(19) United States
(12) Patent Application Publication
(10) Pub. No.: US 2023/0301179 A1
(43) Pub. Date: Sep. 21, 2023

(54) HETEROCYCLIC COMPOUND, ORGANIC LIGHT-EMITTING DEVICE INCLUDING THE SAME, AND ELECTRONIC APPARATUS INCLUDING THE ORGANIC LIGHT-EMITTING DEVICE

(71) Applicants: Samsung Electronics Co., Ltd., Suwon-si (KR); Research & Business Foundation SUNGKYUNKWAN UNIVERSITY, Suwon-si (KR)

(72) Inventors: Soonok JEON, Suwon-si (KR); Jihoon KANG, Suwon-si (KR); Junyeob LEE, Suwon-si (KR); Halim LEE, Suwon-si (KR); Inkoo KIM, Suwon-si (KR); Joonghyuk KIM, Seoul (KR); Yongsik JUNG, Seoul (KR); Hyeonho CHOI, Seoul (KR)

(21) Appl. No.: 17/930,434

(22) Filed: Sep. 8, 2022

(30) Foreign Application Priority Data
Mar. 18, 2022 (KR) 10-2022-0034175

(51) Int. Cl. H01L 51/00; C07D 487/22; C09K 11/06

(52) U.S. Cl. CPC H01L 51/0061; C07D 487/22; C09K 11/06; H01L 51/0072; H01L 51/5012

(57) ABSTRACT

Provided are a heterocyclic compound represented by Formula 1, an organic light-emitting device including the same, and an electronic apparatus including the organic light-emitting device:

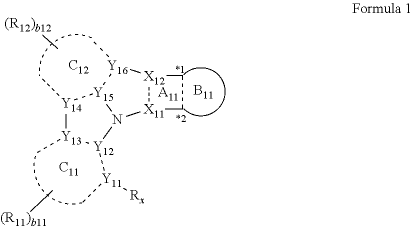

wherein descriptions of Formula 1 are as defined in the present specification.

### CROSS-REFERENCE TO RELATED APPLICATION

This application claims priority under 35 U.S.C. § 119 to Korean Patent Application No. 10-2022-0034175, filed on Mar. 18, 2022, in the Korean Intellectual Property Office, the content of which is incorporated by reference herein in its entirety.

### BACKGROUND

### 1. Field

The present disclosure relates to a heterocyclic compounds, organic light-emitting devices including the same, and electronic apparatuses including the organic light-emitting devices.

### 2. Description of the Related Art

Organic light-emitting devices are self-emissive devices that, as compared with devices in the related art, have wide viewing angles, high contrast ratios, short response times, and excellent brightness, driving voltage, and response speed characteristics, and produce full-color images.

In an example, an organic light-emitting device includes an anode, a cathode, and an organic layer that is arranged between the anode and the cathode and includes an emission layer. A hole transport region may be between the anode and the emission layer, and an electron transport region may be between the emission layer and the cathode. Holes provided from the anode may move toward the emission layer through the hole transport region, and electrons provided from the cathode may move toward the emission layer through the electron transport region. The holes and the electrons recombine in the emission layer to produce excitons. The excitons may transition from an excited state to a ground state, thus generating light.

### SUMMARY

Provided are heterocyclic compounds, organic light-emitting devices including the heterocyclic compounds, and electronic apparatuses including the organic light-emitting devices.

Additional aspects will be set forth in part in the description which follows and, in part, will be apparent from the description, or may be learned by practice of the presented embodiments of the disclosure.

According to an aspect of an embodiment, provided is a heterocyclic compound represented by Formula 1:

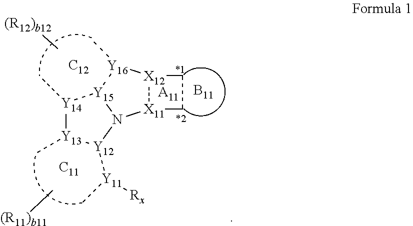

In Formula 1,
X₁₁ and X₁₂ are each independently a carbon atom, and are linked to each other via a single bond or a double bond,
Y₁₁ to Y₁₆ are each independently a carbon atom, and any neighboring two of Y₁₁ to Y₁₆ may be linked via a single bond or a double bond, and
B₁₁ is a group represented by Formula 1A:

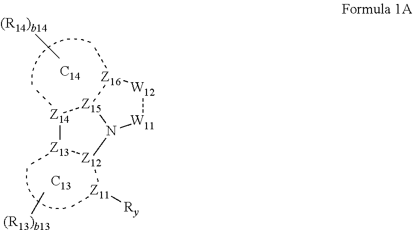

wherein, in Formula 1A,
Z₁₁ to Z₁₆ are each independently a carbon atom, and any neighboring two of Z₁₁ to Z₁₆ are linked via a single bond or a double bond, and
W₁₁ and W₁₂ are each independently a carbon atom, are linked via a single bond or a double bond, and are *1 and *2 in Formula 1,
in Formulae 1 and 1A, A₁₁ and C₁₁ to C₁₄ are each independently a C₅-C₃₀ carbocyclic group or a C₁-C₃₀ heterocyclic group,
Rₓ is a group represented one of by Formulae 2-1 to 2-3,
R_{y} and R₁₁ to R₁₅ are each independently a group represented by one of Formulae 2-1 to 2-3, hydrogen, deuterium, —F, —Cl, —Br, —I, —SF₅, a hydroxyl group, a cyano group, a nitro group, an amidino group, a hydrazino group, a hydrazono group, a substituted or unsubstituted C₁-C₆₀ alkyl group, a substituted or unsubstituted C₂-C₆₀ alkenyl group, a substituted or unsubstituted C₂-C₆₀ alkynyl group, a substituted or unsubstituted C₁-C₆₀ alkoxy group, a substituted or unsubstituted C₃-C₁₀ cycloalkyl group, a substituted or unsubstituted C₁-C₁₀ heterocycloalkyl group, a substituted or unsubstituted C₃-C₁₀ cycloalkenyl group, a substituted or unsubstituted C₂-C₁₀ heterocycloalkenyl group, a substituted or unsubstituted C₆-C₆₀ aryl group, a substituted or unsubstituted C₇-C₆₀ alkylaryl group, a substituted or unsubstituted C₆-C₆₀ aryloxy group, a substituted or unsubstituted C₆-C₆₀ arylthio group, a substituted or unsubstituted C₁-C₆₀ heteroaryl group, a substituted or unsubstituted C₂-C₆₀ heteroalkylaryl group, a substituted or unsubstituted C₁-C₆₀ heteroaryloxy group, a substituted or unsubstituted C₁-C₆₀ heteroarylthio group, a substituted or unsubstituted monovalent non-aromatic condensed polycyclic group, a substituted or unsubstituted monovalent non-aromatic condensed heteropolycyclic group, —Si(Q₁)(Q₂)(Q₃), —C(Q₁)(Q₂)(Q₃), —B(Q₁)(Q₂), —N(Q₁)(Q₂), —P(Q₁)(Q₂), —C(═O)(Q₁), —S(═O)(Q₁), —S(═O)₂(Q₁), —P(═O)(Q₁)(Q₂), or —P(═S)(Q₁)(Q₂),
b11 to b14 are each independently 0, 1, 2, 3, or 4, and
b15 is 0, 1, or 2:

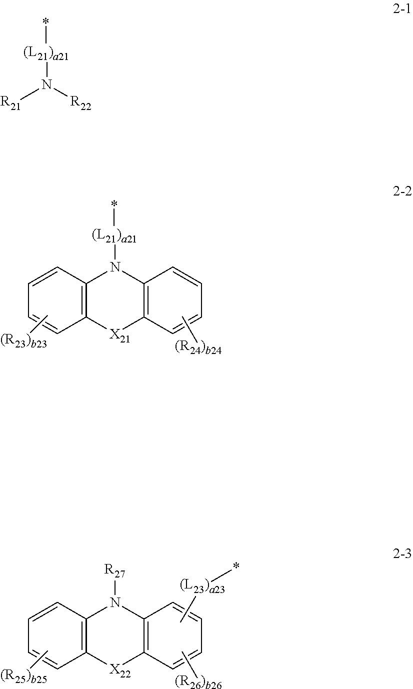

wherein, in Formulae 2-1 to 2-3,
X₂₁ is a single bond, O, S, N(R₂₈), or C(R₂₈)(R₂₉),
X₂₂ is a single bond, O, S, N(R₃₀), or C(R₃₀)(R₃₁),
L₂₁ to L₂₃ are each independently a substituted or unsubstituted C₅-C₃₀ carbocyclic group or a substituted or unsubstituted C₁-C₃₀ heterocyclic group,
a21 to a23 are each independently 0, 1, 2, 3, or 4,
R₂₁ and R₂₂ are each independently a substituted or unsubstituted C₁-C₆₀ alkyl group, a substituted or unsubstituted C₃-C₁₀ cycloalkyl group, a substituted or unsubstituted C₁-C₁₀ heterocycloalkyl group, a substituted or unsubstituted C₃-C₁₀ cycloalkenyl group, a substituted or unsubstituted C₂-C₁₀ heterocycloalkenyl group, a substituted or unsubstituted C₆-C₆₀ aryl group, a substituted or unsubstituted C₇-C₆₀ alkylaryl group, a substituted or unsubstituted C₁-C₆₀ heteroaryl group, a substituted or unsubstituted C₂-C₆₀ heteroalkylaryl group, a substituted or unsubstituted monovalent non-aromatic condensed polycyclic group, or a substituted or unsubstituted monovalent non-aromatic condensed heteropolycyclic group,
R₂₃ to R₃₁ are each independently hydrogen, deuterium, —F, —Cl, —Br, —I, —SF₅, a hydroxyl group, a cyano group, a nitro group, an amidino group, a hydrazine group, a hydrazone group, a substituted or unsubstituted C₁-C₆₀ alkyl group, a substituted or unsubstituted C₂-C₆₀ alkenyl group, a substituted or unsubstituted C₂-C₆₀ alkynyl group, a substituted or unsubstituted C₁-C₆₀ alkoxy group, a substituted or unsubstituted C₃-C₁₀ cycloalkyl group, a substituted or unsubstituted C₁-C₁₀ heterocycloalkyl group, a substituted or unsubstituted C₃-C₁₀ cycloalkenyl group, a substituted or unsubstituted C₂-C₁₀ heterocycloalkenyl group, a substituted or unsubstituted C₆-C₆₀ aryl group, a substituted or unsubstituted C₇-C₆₀ alkylaryl group, a substituted or unsubstituted C₆-C₆₀ aryloxy group, a substituted or unsubstituted C₆-C₆₀ arylthio group, a substituted or unsubstituted C₁-C₆₀ heteroaryl group, a substituted or unsubstituted C₂-C₆₀ heteroalkylaryl group, a substituted or unsubstituted C₁-C₆₀ heteroaryloxy group, a substituted or unsubstituted C₁-C₆₀ heteroarylthio group, a substituted or unsubstituted monovalent non-aromatic condensed polycyclic group, a substituted or unsubstituted monovalent non-aromatic condensed heteropolycyclic group, —Si(Q₁)(Q₂)(Q₃), —C(Q₁)(Q₂)(Q₃), —B(Q₁)(Q₂), —N(Q₁)(Q₂), —P(Q₁)(Q₂), —C(═O)(Q₁), —S(═O)(Q₁), —S(═O)₂(Q₁), —P(═O)(Q₁)(Q₂), or —P(═S)(Q₁)(Q₂),
b23 to b26 are each independently 0, 1, 2, 3, or 4,
Q₁ to Q₃ are each independently hydrogen, deuterium, —F, —Cl, —Br, —I, a hydroxyl group, a cyano group, a nitro group, an amidino group, a hydrazine group, a hydrazone group, a C₁-C₆₀ alkyl group, a C₂-C₆₀ alkenyl group, a C₂-C₆₀ alkynyl group, a C₁-C₆₀ alkoxy group, a C₃-C₁₀ cycloalkyl group, a C₁-C₁₀ heterocycloalkyl group, a C₃-C₁₀ cycloalkenyl group, a C₂-C₁₀ heterocycloalkenyl group, a C₆-C₆₀ aryl group, a C₇-C₆₀ alkylaryl group, a C₆-C₆₀ aryloxy group, a C₆-C₆₀ arylthio group, a C₁-C₆₀ heteroaryl group, a C₂-C₆₀ heteroalkylaryl group, a C₁-C₆₀ heteroaryloxy group, a C₁-C₆₀ heteroarylthio group, a monovalent non-aromatic condensed polycyclic group, a monovalent non-aromatic condensed heteropolycyclic group, a C₁-C₆₀ alkyl group that is substituted with at least one deuterium, —F, a cyano group, a C₁-C₆₀ alkyl group, a C₆-C₆₀ aryl group, or a combination thereof, or a C₆-C₆₀ aryl group that is substituted with deuterium, —F, a cyano group, a C₁-C₆₀ alkyl group, a C₆-C₆₀ aryl group, or a combination thereof, and
* indicates a binding site to a neighboring atom.

According to an aspect of another embodiment, provided is an organic light-emitting device including: a first electrode; a second electrode; an organic layer arranged between the first electrode and the second electrode and including an emission layer; and the heterocyclic compound.

According to an aspect of another embodiment, provided is an electronic apparatus including the light-emitting device.

### BRIEF DESCRIPTION OF THE DRAWINGS

The above and other aspects, features, and advantages of certain embodiments of the disclosure will be more apparent from the following description taken in conjunction with the accompanying drawings, in which:

FIG. 1 is a schematic cross-sectional view of an organic light-emitting device 10 according to an exemplary embodiment;

FIG. 2 is a schematic cross-sectional view of an organic light-emitting device 100 according to another exemplary embodiment;

FIG. 3 is a schematic cross-sectional view of an organic light-emitting device 200 according to an exemplary embodiment; and

FIGS. 4A to 4E each show a diagram schematically illustrating energy transfer in an emission layer of an organic light-emitting device according to an exemplary embodiment.

### DETAILED DESCRIPTION

Reference will now be made in detail to embodiments, examples of which are illustrated in the accompanying drawings, wherein like reference numerals refer to like elements throughout. In this regard, the present embodiments may have different forms and should not be construed as being limited to the descriptions set forth herein. Accordingly, the embodiments are merely described below, by referring to the figures, to explain aspects. As used herein, the term “and/or” includes any and all combinations of one or more of the associated listed items. Expressions such as “at least one of,” when preceding a list of elements, modify the entire list of elements and do not modify the individual elements of the list.

It will be understood that when an element is referred to as being “on” another element, it can be directly on the other element or intervening elements may be present therebetween In contrast, when an element is referred to as being “directly on” another element, there are no intervening elements present

It will be understood that, although the terms “first,” “second,” “third” etc. may be used herein to describe various elements, components, regions, layers and/or sections, these elements, components, regions, layers and/or sections should not be limited by these terms. These terms are only used to distinguish one element, component, region, layer or section from another element, component, region, layer or section. Thus, “a first element,” “component,” “region,” “layer” or “section” discussed below could be termed a second element, component, region, layer or section without departing from the teachings herein.

The terminology used herein is for the purpose of describing particular embodiments only and is not intended to be limiting. As used herein, “a,” “an,” “the,” and “at least one” do not denote a limitation of quantity, and are intended to cover both the singular and plural, unless the context clearly indicates otherwise. For example, “an element” has the same meaning as “at least one element,” unless the context clearly indicates otherwise.

“Or” means “and/or.” As used herein, the term “and/or” includes any and all combinations of one or more of the associated listed items. It will be further understood that the terms “comprises” and/or “comprising,” or “includes” and/or “including” when used in this specification, specify the presence of stated features, regions, integers, steps, operations, elements, and/or components, but do not preclude the presence or addition of one or more other features, regions, integers, steps, operations, elements, components, and/or groups thereof.

Furthermore, relative terms, such as “lower” or “bottom” and “upper” or “top,” may be used herein to describe one element's relationship to another element as illustrated in the Figures. It will be understood that relative terms are intended to encompass different orientations of the device in addition to the orientation depicted in the Figures. For example, if the device in one of the figures is turned over, elements described as being on the “lower” side of other elements would then be oriented on “upper” sides of the other elements. The exemplary term “lower,” can therefore, encompasses both an orientation of “lower” and “upper,” depending on the particular orientation of the figure. Similarly, if the device in one of the figures is turned over, elements described as “below” or “beneath” other elements would then be oriented “above” the other elements. The exemplary terms “below” or “beneath” can, therefore, encompass both an orientation of above and below.

“About” or “approximately” as used herein is inclusive of the stated value and means within an acceptable range of deviation for the particular value as determined by one of ordinary skill in the art, considering the measurement in question and the error associated with measurement of the particular quantity (i.e., the limitations of the measurement system). For example, “about” can mean within one or more standard deviations, or within ±30%, 20%, 10% or 5% of the stated value.

Unless otherwise defined, all terms (including technical and scientific terms) used herein have the same meaning as commonly understood by one of ordinary skill in the art to which this disclosure belongs. It will be further understood that terms, such as those defined in commonly used dictionaries, should be interpreted as having a meaning that is consistent with their meaning in the context of the relevant art and the present disclosure, and will not be interpreted in an idealized or overly formal sense unless expressly so defined herein.

Exemplary embodiments are described herein with reference to cross section illustrations that are schematic illustrations of idealized embodiments. As such, variations from the shapes of the illustrations as a result, for example, of manufacturing techniques and/or tolerances, are to be expected. Thus, embodiments described herein should not be construed as limited to the particular shapes of regions as illustrated herein but are to include deviations in shapes that result, for example, from manufacturing. For example, a region illustrated or described as flat may, typically, have rough and/or nonlinear features. Moreover, sharp angles that are illustrated may be rounded Thus, the regions illustrated in the figures are schematic in nature and their shapes are not intended to illustrate the precise shape of a region and are not intended to limit the scope of the present claims.

An aspect of the present disclosure provides a heterocyclic compound represented by Formula 1:

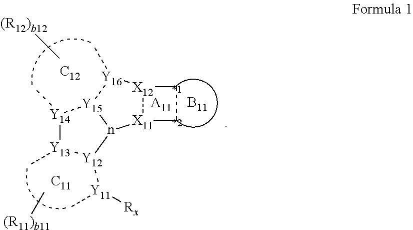

In Formula 1, X₁₁ and X₁₂ may each independently be a carbon atom, and may be linked to each other via a single bond or a double bond.

In Formula 1, Y₁₁ to Y₁₆ may each independently be a carbon atom, and any neighboring two of Y₁₁ to Y₁₆ may be linked via a single bond or a double bond.

In Formula 1, B₁₁ may be a group represented by Formula 1A:

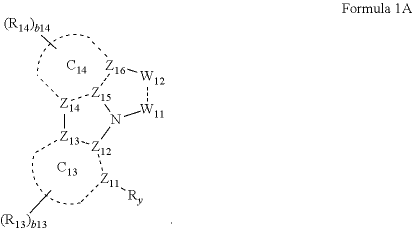

In Formula 1A, Z₁₁ to Z₁₆ may each independently be a carbon atom, and any neighboring two of Z₁₁ to Z₁₆ may be linked via a single bond or a double bond.

In Formula 1A, W₁₁ and W₁₂ may each independently be a carbon atom, may be linked via a single bond or a double bond, and may be *1 and *2 in Formula 1.

For example, in Formulae 1 and 1A, W₁₁ may be *1 and W₁₂ may be *2; or may be *2, and W₁₂ may be *1.

In Formulae 1 and 1A, A₁₁ and C₁₁ to C₁₄ may each independently be a substituted or unsubstituted C₅-C₃₀ carbocyclic group or a substituted or unsubstituted C₁-C₃₀ heterocyclic group.

In an embodiment, in Formulae 1 and 1A, A₁₁ and C₁₁ to C₁₄ may each independently be a benzene group, a naphthalene group, a phenanthrene group, a furan group, a thiophene group, a pyrrole group, a cyclopentene group, a silole group, a germole group, a benzofuran group, a benzothiophene group, an indole group, an indene group, a benzosilole group, a benzogermole group, a dibenzofuran group, a dibenzothiophene group, a carbazole group, a fluorene group, a dibenzosilole group, a dibenzogermole group, a pyridine group, a pyrimidine group, or a pyridazine group.

In one or more embodiments, in Formulae 1 and 1A, A₁₁ and C₁₁ to C₁₄ may each independently be a benzene group, a naphthalene group, a benzofuran group, a benzothiophene group, an indole group, an indene group, a benzosilole group, a benzogermole group, a pyridine group, or a pyridazine group.

In one or more embodiments, in Formulae 1 and 1A, A₁₁ and C₁₁ to C₁₄ may each independently be a benzene group, a naphthalene group, a pyridine group, or a pyridazine group.

For example, A₁₁ in Formulae 1 and 1A may be a group represented by Formula 3-1:

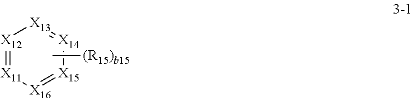

wherein, in Formula 3-1,
X₁₁ to X₁₆ may each independently be a nitrogen atom or a carbon atom, and any neighboring two of X₁₃ to X₁₆ may each be *1 and *2 in Formula 1,
R₁₅ may be defined the same as R₁₁ in Formula 1, and
b15 may be 0, 1, or 2.

For example, in Formulae 1 and 1A, A₁₁ may be a group represented by one of Formulae 3-11 to 3-16:

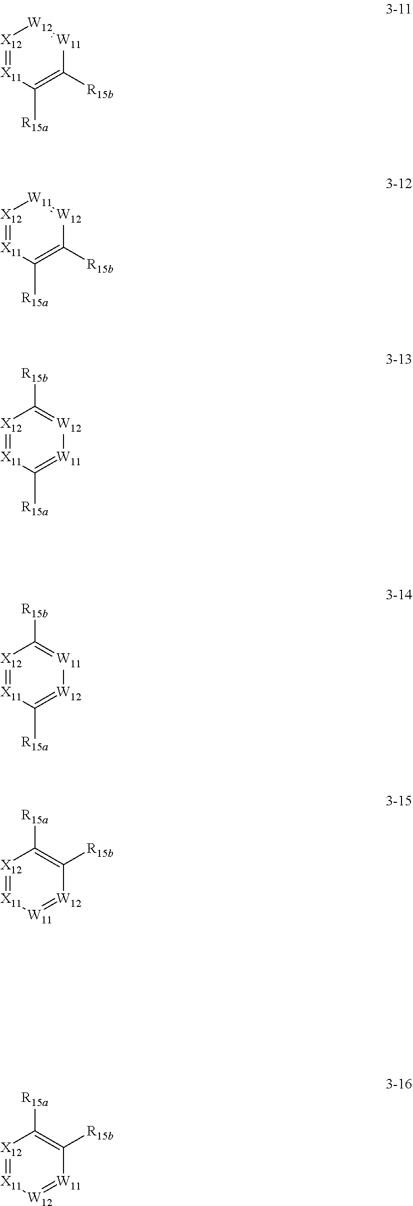

wherein, in Formulae 3-11 to 3-16,
X₁₁ to X₁₆ may each independently be a nitrogen atom or a carbon atom,
W₁₁ and W₁₂ may each independently be a nitrogen atom or a carbon atom, and
R₁₅ₐ and R_{16b} may each independently be the same as R₁₁ in Formula 1.

In Formula 1, Rₓ may be a group represented by one of Formulae 2-1 to 2-3:

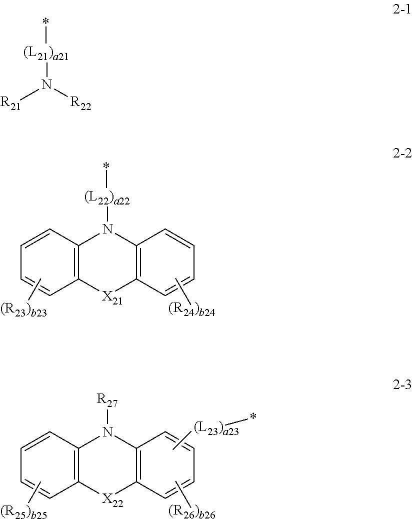

wherein, in Formulae 2-1 to 2-3,
X₂₁ may be a single bond, O, S, N(R₂₈), or C(R₂₈)(R₂₉),
X₂₂ may be a single bond, O, S, N(R₃₀), or C(R₃₀)(R₃₁),
L₂₁ to L₂₃ may each independently be a substituted or unsubstituted C₅-C₃₀ carbocyclic group or a substituted or unsubstituted C₁-C₃₀ heterocyclic group,
a21 to a23 may each independently be 0, 1, 2, 3, or 4,
R₂₁ and R₂₂ may each independently be a substituted or unsubstituted C₁-C₆₀ alkyl group, a substituted or unsubstituted C₃-C₁₀ cycloalkyl group, a substituted or unsubstituted C₁-C₁₀ heterocycloalkyl group, a substituted or unsubstituted C₃-C₁₀ cycloalkenyl group, a substituted or unsubstituted C₂-C₁₀ heterocycloalkenyl group, a substituted or unsubstituted C₆-C₆₀ aryl group, a substituted or unsubstituted C₇-C₆₀ alkylaryl group, a substituted or unsubstituted C₁-C₆₀ heteroaryl group, a substituted or unsubstituted C₂-C₆₀ heteroalkylaryl group, a substituted or unsubstituted monovalent non-aromatic condensed polycyclic group, or a substituted or unsubstituted monovalent non-aromatic condensed heteropolycyclic group,
R₂₃ to R₃₁ may each independently be hydrogen, deuterium, —F, —Cl, —Br, —I, —SF₅, a hydroxyl group, a cyano group, a nitro group, an amidino group, a hydrazine group, a hydrazone group, a substituted or unsubstituted C₁-C₆₀ alkyl group, a substituted or unsubstituted C₂-C₆₀ alkenyl group, a substituted or unsubstituted C₂-C₆₀ alkynyl group, a substituted or unsubstituted C₁-C₆₀ alkoxy group, a substituted or unsubstituted C₃-C₁₀ cycloalkyl group, a substituted or unsubstituted C₁-C₁₀ heterocycloalkyl group, a substituted or unsubstituted C₃-C₁₀ cycloalkenyl group, a substituted or unsubstituted C₂-C₁₀ heterocycloalkenyl group, a substituted or unsubstituted C₆-C₆₀ aryl group, a substituted or unsubstituted C₇-C₆₀ alkylaryl group, a substituted or unsubstituted C₆-C₆₀ aryloxy group, a substituted or unsubstituted C₆-C₆₀ arylthio group, a substituted or unsubstituted C₁-C₆₀ heteroaryl group, a substituted or unsubstituted C₂-C₆₀ heteroalkylaryl group, a substituted or unsubstituted C₁-C₆₀ heteroaryloxy group, a substituted or unsubstituted C₁-C₆₀ heteroarylthio group, a substituted or unsubstituted monovalent non-aromatic condensed polycyclic group, a substituted or unsubstituted monovalent non-aromatic condensed heteropolycyclic group, —Si(Q₁)(Q₂)(Q₃), —C(Q₁)(Q₂)(Q₃), —B(Q₁)(Q₂), —N(Q₁)(Q₂), —P(Q₁)(Q₂), —C(═O)(Q₁), —S(═O)(Q₁), —S(═O)₂(Q₁), —P(═O)(Q₁)(Q₂), or —P(═S)(Q₁)(Q₂),
b23 to b26 may each independently be 0, 1, 2, 3, or 4,
Q₁ to Q₃ may each independently be hydrogen, deuterium, —F, —Cl, —Br, —I, a hydroxyl group, a cyano group, a nitro group, an amidino group, a hydrazine group, a hydrazone group, a C₁-C₆₀ alkyl group, a C₂-C₆₀ alkenyl group, a C₂-C₆₀ alkynyl group, a C₁-C₆₀ alkoxy group, a C₃-C₁₀ cycloalkyl group, a C₁-C₁₀ heterocycloalkyl group, a C₃-C₁₀ cycloalkenyl group, a C₂-C₁₀ heterocycloalkenyl group, a C₆-C₆₀ aryl group, a C₇-C₆₀ alkylaryl group, a C₆-C₆₀ aryloxy group, a C₆-C₆₀ arylthio group, a C₁-C₆₀ heteroaryl group, a C₂-C₆₀ heteroalkylaryl group, a C₁-C₆₀ heteroaryloxy group, a C₁-C₆₀ heteroarylthio group, a monovalent non-aromatic condensed polycyclic group, a monovalent non-aromatic condensed heteropolycyclic group, a C₁-C₆₀ alkyl group that is substituted with at least one deuterium, —F, a cyano group, a C₁-C₆₀ alkyl group, a C₆-C₆₀ aryl group, or a combination thereof, or a C₆-C₆₀ aryl group that is substituted with deuterium, —F, a cyano group, a C₁-C₆₀ alkyl group, a C₆-C₆₀ aryl group, or a combination thereof, and
* indicates a binding site to a neighboring atom.

In an embodiment, in Formulae 2-1 to 2-3, X₂₁ may be a single bond, and X₂₂ may be a single bond.

In an embodiment, in Formulae 2-1 to 2-3, L₂₁ and L₂₂ may each independently be a substituted or unsubstituted C₅-C₃₀ carbocyclic group.

In one or more embodiments, in Formulae 2-1 to 2-3, L₂₁ and L₂₂ may each independently be a benzene group.

In an embodiment, in Formulae 2-1 to 2-3, a21 and a22 may each independently be 0 or 1.

In an embodiment, in Formulae 2-1 to 2-3, R₂₁ and R₂₂ may each independently be: a C₁-C₂₀ alkyl group unsubstituted or substituted with deuterium, —F, —Cl, —Br, —I, —CD₃, —CD₂H, —CDH₂, —CF₃, —CF₂H, —CFH₂, a hydroxyl group, a cyano group, a nitro group, a C₁-C₂₀ alkyl group, a deuterated C₂-C₂₀ alkyl group, a C₁-C₂₀ alkoxy group, a cyclopentyl group, a cyclohexyl group, a cycloheptyl group, a cyclooctyl group, an adamantanyl group, a norbornanyl group, a norbornenyl group, a cyclopentenyl group, a cyclohexenyl group, a cycloheptenyl group, a bicyclo[1.1.1]pentyl group, a bicyclo[2.1.1]hexyl group, a bicyclo[2.2.1]heptyl group, a bicyclo[2.2.2]octyl group, a (C₁-C₂₀ alkyl)cyclopentyl group, a (C₁-C₂₀ alkyl)cyclohexyl group, a (C₁-C₂₀ alkyl)cycloheptyl group, a (C₁-C₂₀ alkyl)cyclooctyl group, a (C₁-C₂₀ alkyl)adamantanyl group, a (C₁-C₂₀ alkyl)norbornanyl group, a (C₁-C₂₀ alkyl)norbornenyl group, a (C₁-C₂₀ alkyl)cyclopentenyl group, a (C₁-C₂₀ alkyl) cyclohexenyl group, a (C₁-C₂₀ alkyl)cycloheptenyl group, a (C₁-C₂₀ alkyl)bicyclo[1.1.1]pentyl group, a (C₁-C₂₀ alkyl)bicyclo[2.1.1]hexyl group, a (C₁-C₂₀ alkyl)bicyclo[2.2.1]heptyl group, a (C₁-C₂₀ alkyl)bicyclo[2.2.2]octyl group, a phenyl group, a (C₁-C₂₀ alkyl)phenyl group, a biphenyl group, a terphenyl group, a naphthyl group, —Si(Q₁₁)(Q₁₂)(Q₁₃), —Ge(Q₁₁)(Q₁₂)(Q₁₃), —C(Q₁₁)(Q₁₂)(Q₁₃), —B(Q₁₁)(Q₁₂), —N(Q₁₁)(Q₁₂), —P(Q₁₁)(Q₁₂), —C(═O)(Q₁₁), —S(═O)(Q₁₁), —S(═O)₂(Q₁₁), —P(═O)(Q₁₁)(Q₁₂), —P(═S)(Q₁₁)(12), or any combination thereof; or
a cyclopentyl group, a cyclohexyl group, a cycloheptyl group, a cyclooctyl group, an adamantanyl group, a norbornanyl group, a norbornenyl group, a cyclopentenyl group, a cyclohexenyl group, a cycloheptenyl group, a bicyclo[1.1.1]pentyl group, a bicyclo[2.1.1]hexyl group, a bicyclo[2.2.1]heptyl group, a bicyclo[2.2.2]octyl group, a phenyl group, a (C₁-C₂₀ alkyl)phenyl group, a biphenyl group, a terphenyl group, a naphthyl group, a fluorenyl group, a phenanthrenyl group, an anthracenyl group, a fluoranthenyl group, a triphenylenyl group, a pyrenyl group, a chrysenyl group, a pyrrolyl group, a thiophenyl group, a furanyl group, an imidazolyl group, a pyrazolyl group, a thiazolyl group, an isothiazolyl group, an oxazolyl group, an isoxazolyl group, a pyridinyl group, a pyrazinyl group, a pyrimidinyl group, a pyridazinyl group, an isoindolyl group, an indolyl group, an indazolyl group, a purinyl group, a quinolinyl group, an isoquinolinyl group, a benzoquinolinyl group, a quinoxalinyl group, a quinazolinyl group, a cinnolinyl group, a carbazolyl group, a phenanthrolinyl group, a benzimidazolyl group, a benzofuranyl group, a benzothiophenyl group, an isobenzothiazolyl group, a benzoxazolyl group, an isobenzoxazolyl group, a triazolyl group, a tetrazolyl group, an oxadiazolyl group, a triazinyl group, a dibenzofuranyl group, a dibenzothiophenyl group, a benzocarbazolyl group, a dibenzocarbazolyl group, an imidazopyridinyl group, an imidazopyrimidinyl group, an azacarbazolyl group, an azadibenzofuranyl group, or an azadibenzothiophenyl group, each unsubstituted or substituted with deuterium, —F, —Cl, —Br, —I, —CD₃, —CD₂H, —CDH₂, —CF₃, —CF₂H, —CFH₂, a hydroxyl group, a cyano group, a nitro group, a C₁-C₂₀ alkyl group, a deuterated C₂-C₂₀ alkyl group, a C₁-C₂₀ alkoxy group, a cyclopentyl group, a cyclohexyl group, a cycloheptyl group, a cyclooctyl group, an adamantanyl group, a norbornanyl group, a norbornenyl group, a cyclopentenyl group, a cyclohexenyl group, a cycloheptenyl group, a bicyclo[1.1.1]pentyl group, a bicyclo[2.1.1]hexyl group, a bicyclo[2.2.1]heptyl group, a bicyclo[2.2.2]octyl group, a (C₁-C₂₀ alkyl)cyclopentyl group, a (C₁-C₂₀ alkyl)cyclohexyl group, a (C₁-C₂₀ alkyl)cycloheptyl group, a (C₁-C₂₀ alkyl)cyclooctyl group, a (C₁-C₂₀ alkyl)adamantanyl group, a (C₁-C₂₀ alkyl)norbornanyl group, a (C₁-C₂₀ alkyl)norbornenyl group, a (C₁-C₂₀ alkyl)cyclopentenyl group, a (C₁-C₂₀ alkyl)cyclohexenyl group, a (C₁-C₂₀ alkyl)cycloheptenyl group, a (C₁-C₂₀ alkyl)bicyclo[1.1.1]pentyl group, a (C₁-C₂₀ alkyl)bicyclo[2.1.1]hexyl group, a (C₁-C₂₀ alkyl)bicyclo[2.2.1]heptyl group, a (C₁-C₂₀ alkyl)bicyclo[2.2.2]octyl group, a phenyl group, a (C₁-C₂₀ alkyl)phenyl group, a biphenyl group, a terphenyl group, a naphthyl group, a pyridinyl group, a pyrazinyl group, a pyrimidinyl group, a pyridazinyl group, a quinolinyl group, an isoquinolinyl group, —Si(Q₂₁)(Q₂₂)(Q₂₃), —Ge(Q₂₁)(Q₂₂)(Q₂₃), —C(Q₂₁)(Q₂₂)(Q₂₃), —B(Q₂₁)(Q₂₂), —N(Q₂₁)(Q₂₂), —P(Q₂₁)(Q₂₂), —C(═O)(Q₂₁), —S(═O)(Q₂₁), —S(═O)₂(Q₂₁), —P(═O)(Q₂₁)(Q₂₂), —P(═S)(Q₂₁)(Q₂₂), or any combination thereof, and
Q₁₁ to Q₁₃ and Q₂₁ to Q₂₃ may each independently be: deuterium, —F, —CH₃, —CD₃, —CD₂H, —CDH₂, —CH₂CH₃, —CH₂CD₃, —CH₂CD₂H, —CH₂CDH₂, —CHDCH₃, —CHDCD₂H, —CHDCDH₂, —CHDCD₃, —CD₂CD₃, —CD₂CD₂H, —CD₂CDH₂, —CF₃, —CF₂H, —CFH₂, —CH₂CF₃, —CH₂CF₂H, —CH₂CFH₂, —CHFCH₃, —CHFCF₂H, —CHFCFH₂, —CHFCF₃, —CF₂CF₃, —CF₂CF₂H, or —CF₂CFH₂; or
an n-propyl group, an iso-propyl group, an n-butyl group, a sec-butyl group, an isobutyl group, a tert-butyl group, an n-pentyl group, a tert-pentyl group, a neopentyl group, an isopentyl group, a sec-pentyl group, a 3-pentyl group, a sec-isopentyl group, a phenyl group, a biphenyl group, or a naphthyl group, each unsubstituted or substituted with deuterium, —F, a C₁-C₁₀ alkyl group, a deuterated C₁-C₁₀ alkyl group, a phenyl group, or any combination thereof.

In one or more embodiments, in Formulae 2-1 to 2-3, R₂₁ and R₂₂ may each independently be: a phenyl group, a (C₁-C₂₀ alkyl)phenyl group, a biphenyl group, a terphenyl group, a naphthyl group, a fluorenyl group, a phenanthrenyl group, an anthracenyl group, a fluoranthenyl group, a triphenylenyl group, a pyrenyl group, a chrysenyl group, a thiophenyl group, a furanyl group, a carbazolyl group, a benzofuranyl group, a benzothiophenyl group, a dibenzofuranyl group, a dibenzothiophenyl group, a benzocarbazolyl group, or a dibenzocarbazolyl group, each unsubstituted or substituted with deuterium, —F, —Cl, —Br, —I, —CD₃, —CD₂H, —CDH₂, —CF₃, —CF₂H, —CFH₂, a hydroxyl group, a cyano group, a nitro group, a C₁-C₂₀ alkyl group, a deuterated C₂-C₂₀ alkyl group, a C₁-C₂₀ alkoxy group, a cyclopentyl group, a cyclohexyl group, a cycloheptyl group, a cyclooctyl group, an adamantanyl group, a norbornanyl group, a norbornenyl group, a cyclopentenyl group, a cyclohexenyl group, a cycloheptenyl group, a bicyclo[1.1.1]pentyl group, a bicyclo[2.1.1]hexyl group, a bicyclo[2.2.1]heptyl group, a bicyclo[2.2.2]octyl group, a (C₁-C₂₀ alkyl)cyclopentyl group, a (C₁-C₂₀ alkyl)cyclohexyl group, a (C₁-C₂₀ alkyl)cycloheptyl group, a (C₁-C₂₀ alkyl)cyclooctyl group, a (C₁-C₂₀ alkyl)adamantanyl group, a (C₁-C₂₀ alkyl)norbornanyl group, a (C₁-C₂₀ alkyl)norbornenyl group, a (C₁-C₂₀ alkyl)cyclopentenyl group, a (C₁-C₂₀ alkyl)cyclohexenyl group, a (C₁-C₂₀ alkyl)cycloheptenyl group, a (C₁-C₂₀ alkyl)bicyclo[1.1.1]pentyl group, a (C₁-C₂₀ alkyl)bicyclo[2.1.1]hexyl group, a (C₁-C₂₀ alkyl)bicyclo[2.2.1]heptyl group, a (C₁-C₂₀ alkyl)bicyclo[2.2.2]octyl group, a phenyl group, a (C₁-C₂₀ alkyl)phenyl group, a biphenyl group, a terphenyl group, a naphthyl group, or any combination thereof.

In one or more embodiments, in Formulae 2-1 to 2-3, R₂₁ and R₂₂ may each independently be —CH₃, —CD₃, —CD₂H, —CDH₂, —CF₃, —CF₂H, —CFH₂, a group represented by one of Formulae 10-12 to 10-23, 10-38 to 10-130, and 10-238 to 10-272, a group represented by one of Formulae 10-12 to 10-23, 10-38 to 10-130, and 10-238 to 10-272 in which at least one hydrogen is substituted with deuterium, a group represented by one of Formulae 10-12 to 10-23, 10-38 to 10-130, and 10-238 to 10-272 in which at least one hydrogen is substituted with —F:

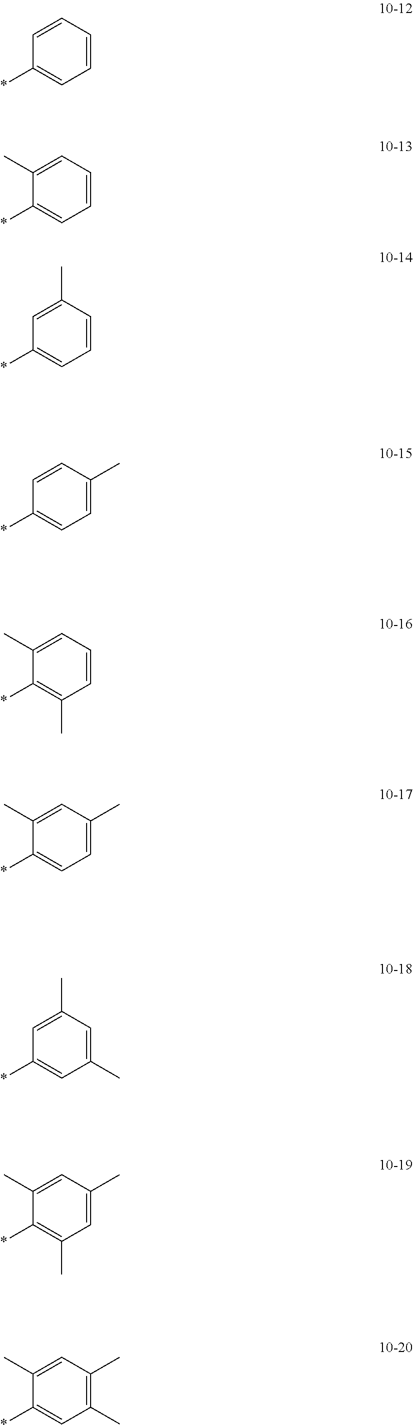

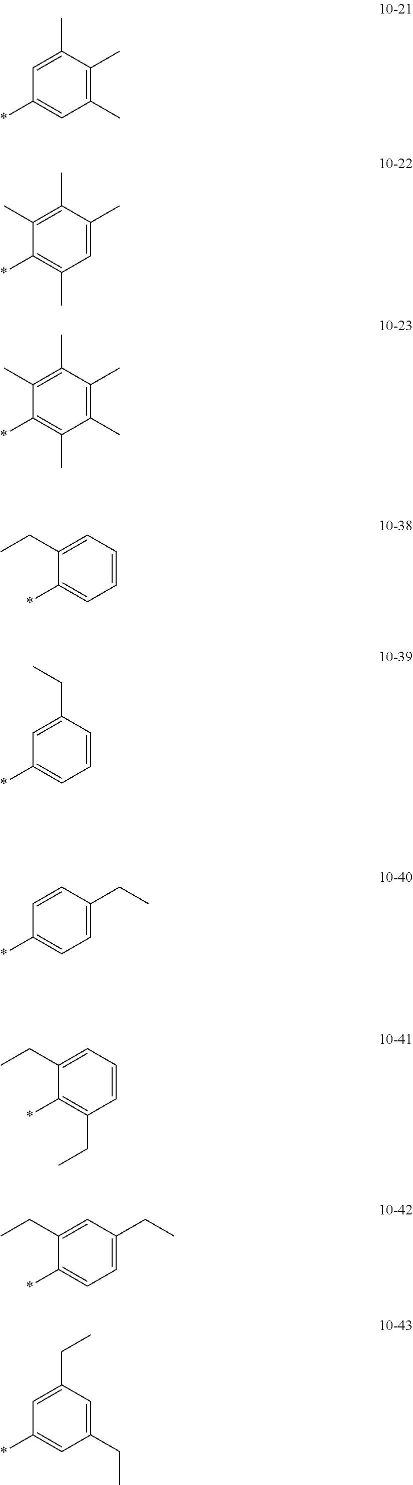

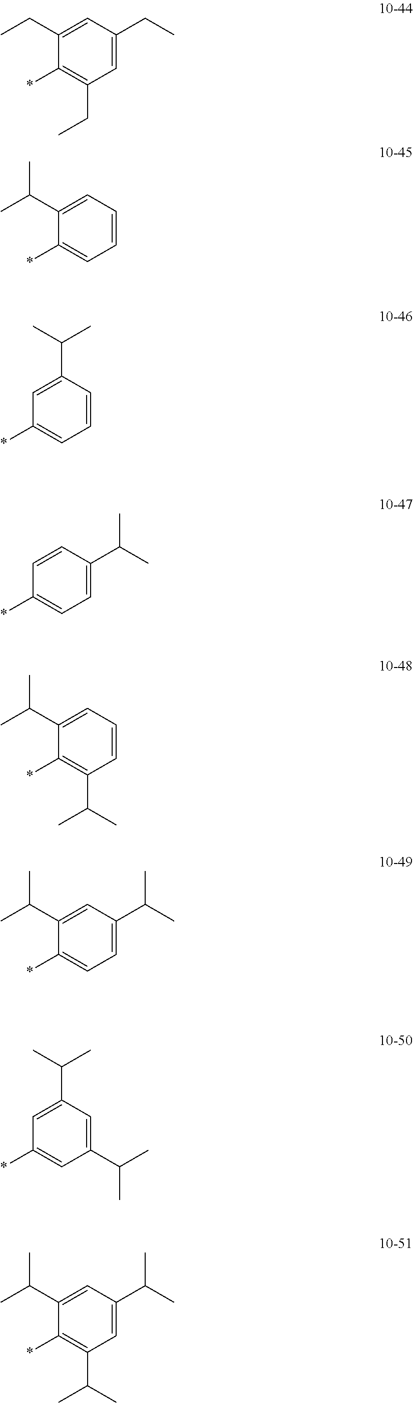

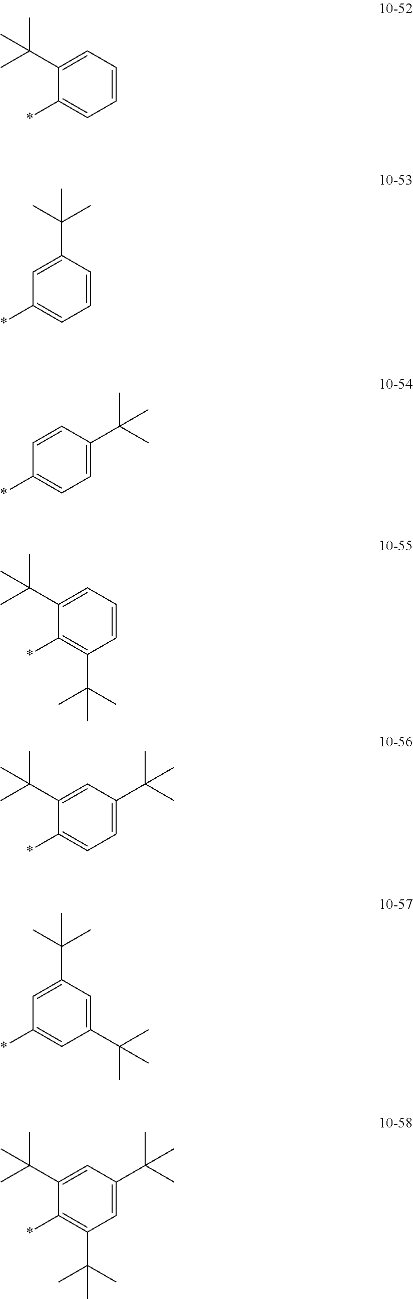

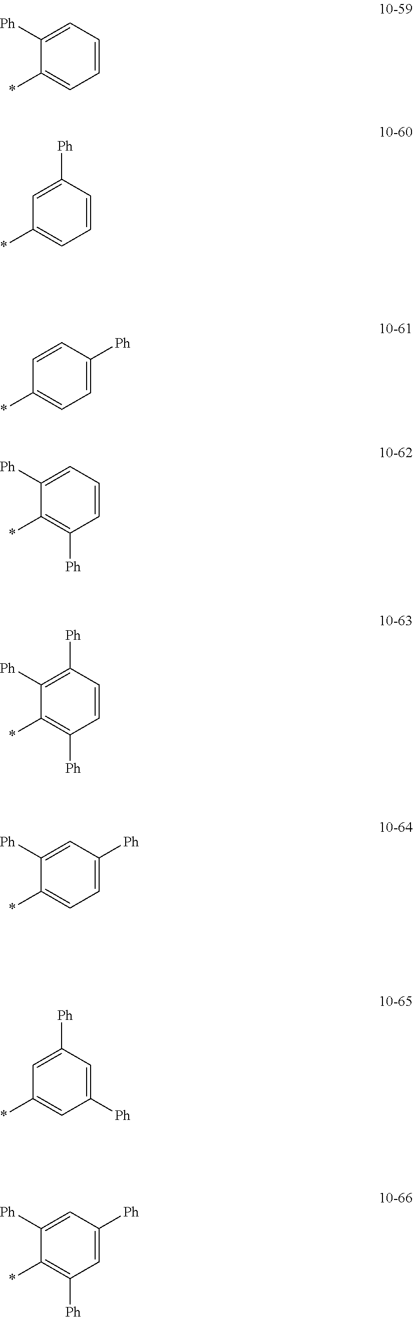

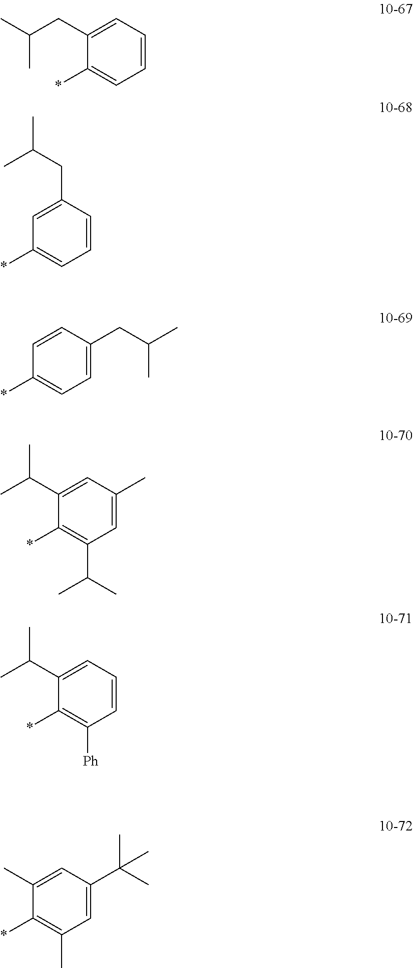

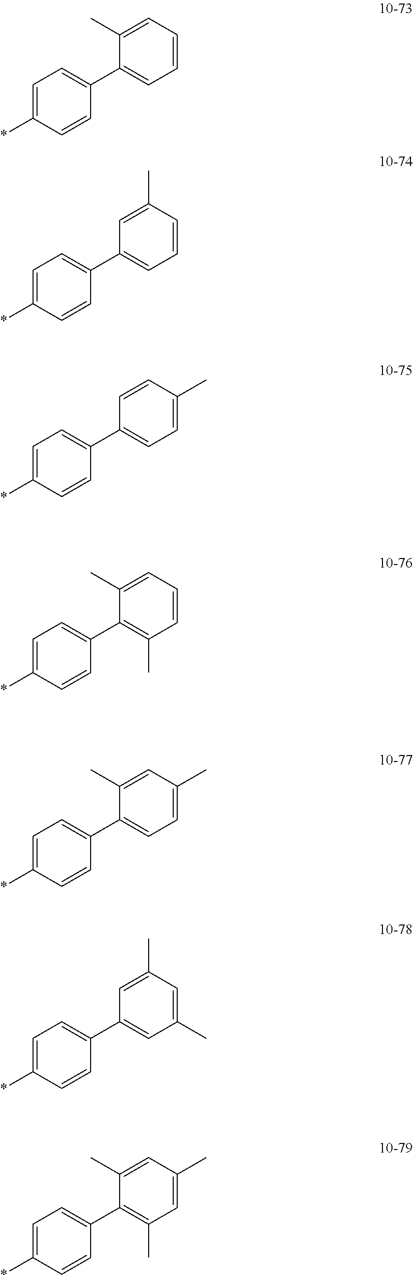

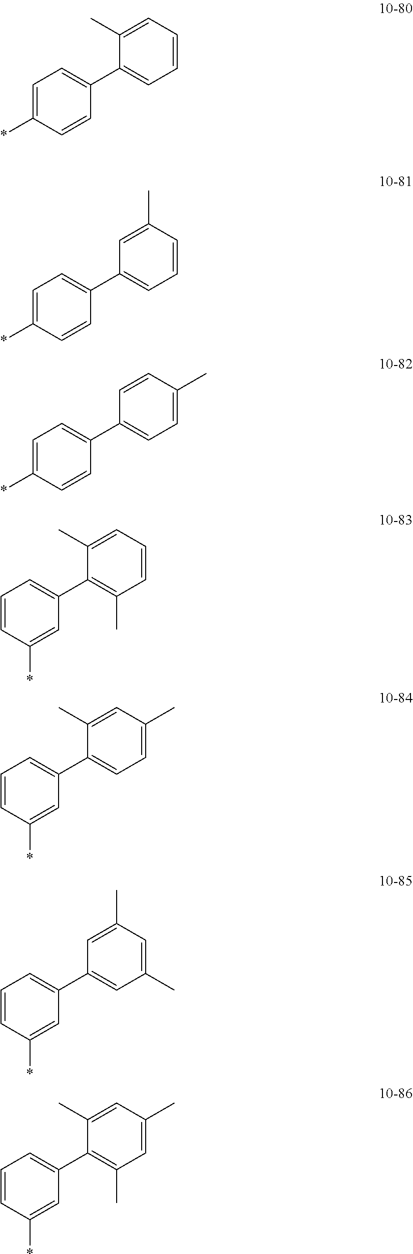

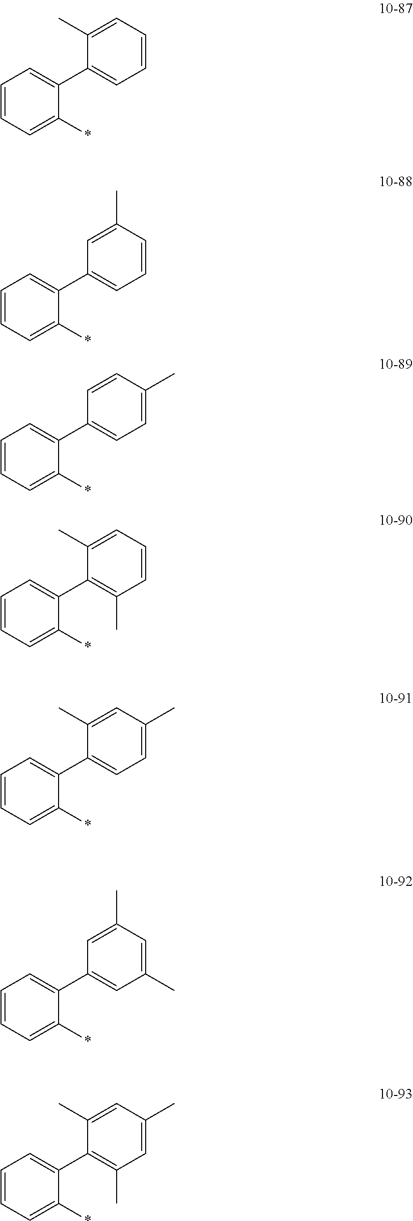

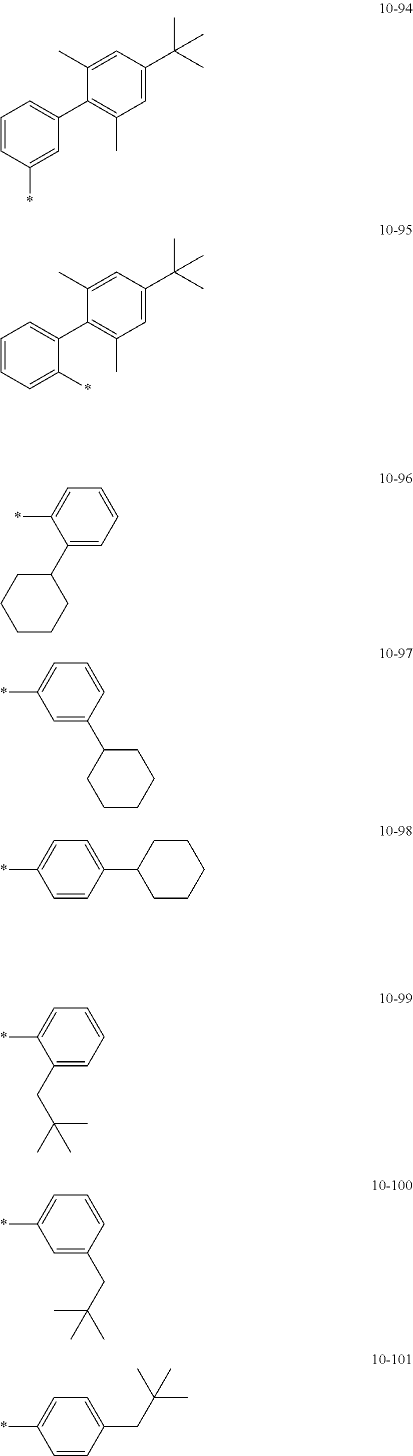

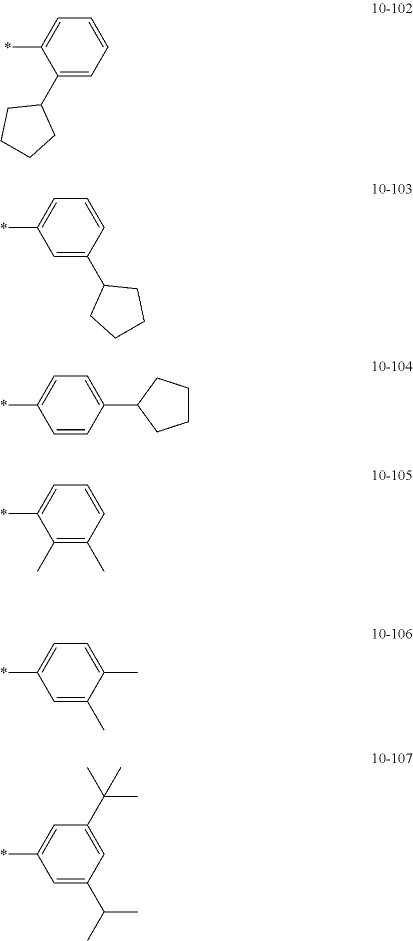

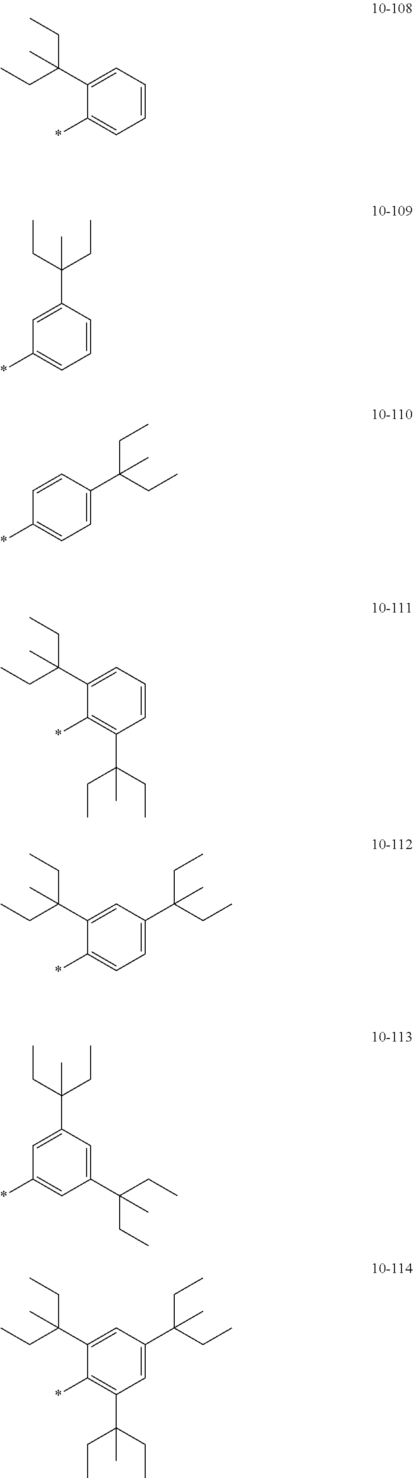

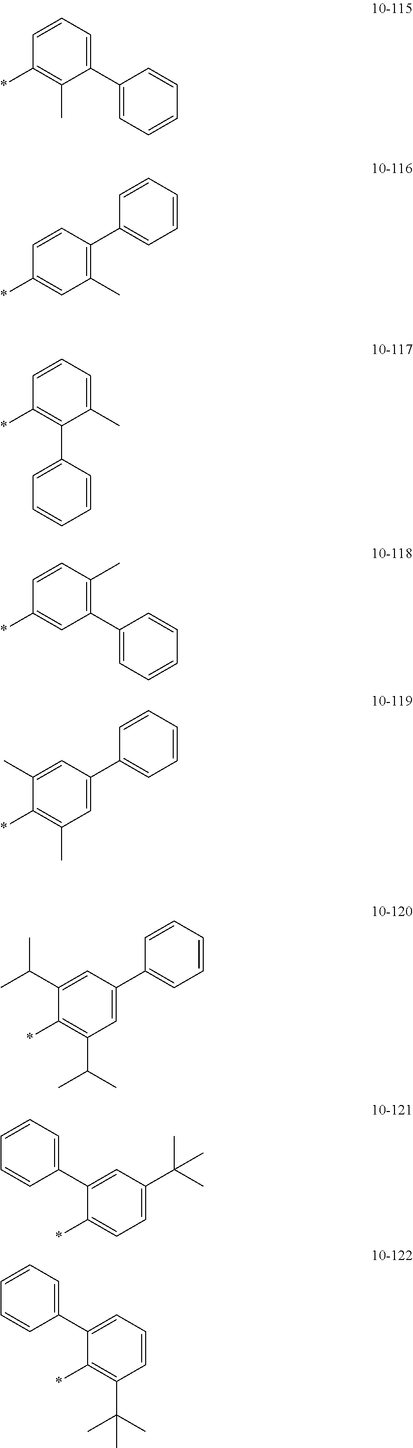

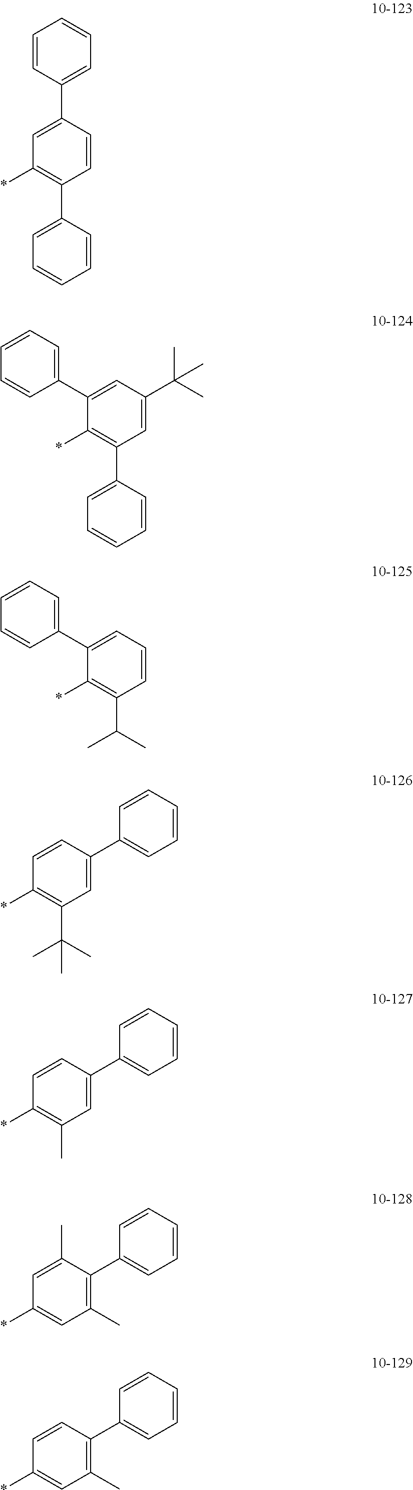

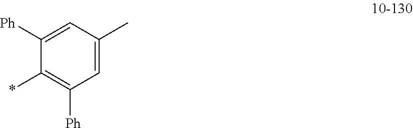 [SMILES: *c1c(-c2ccccc2)cc(C)cc1-c1ccccc1]

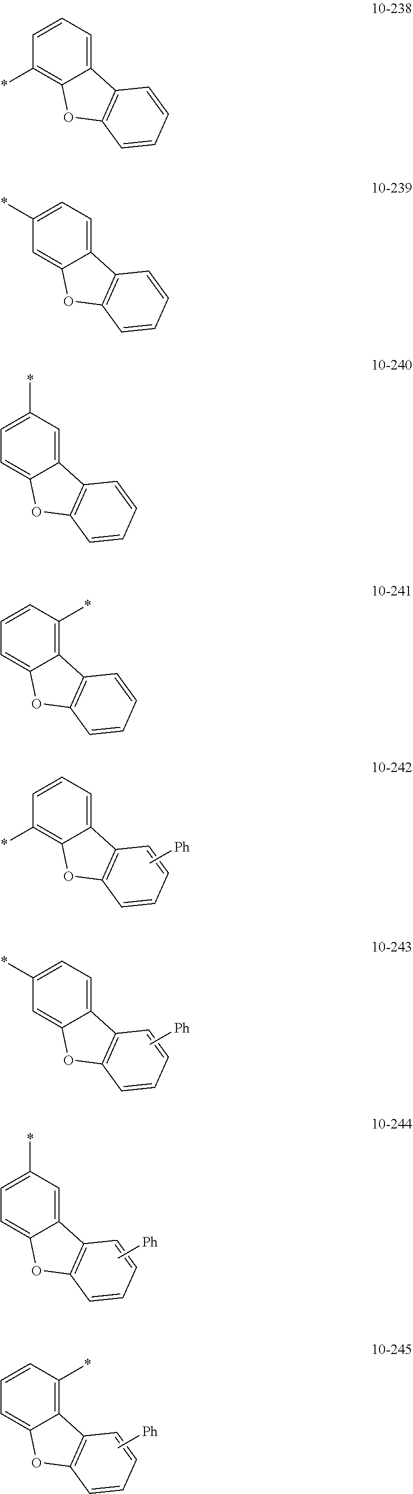

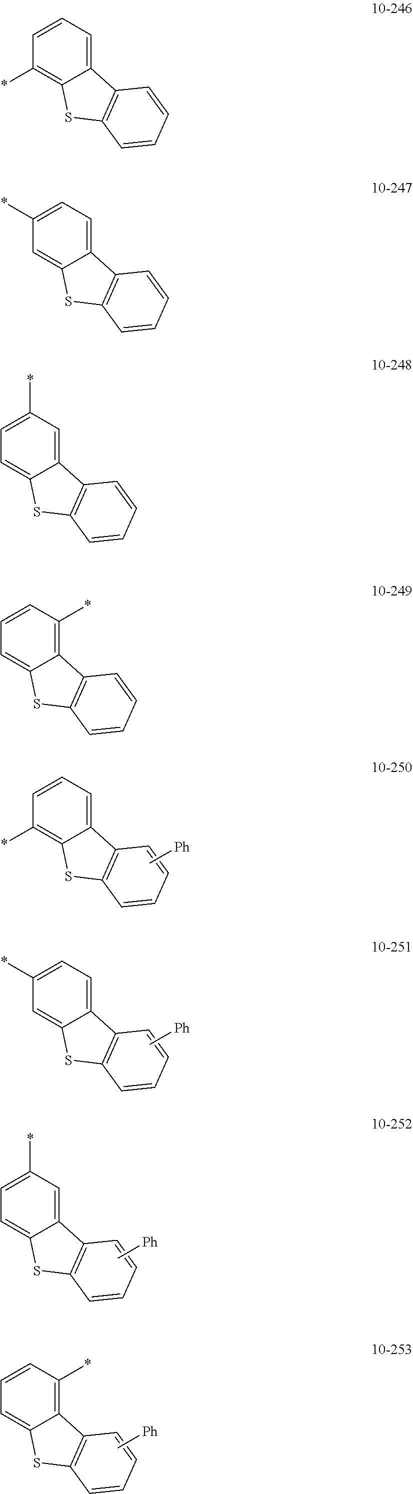

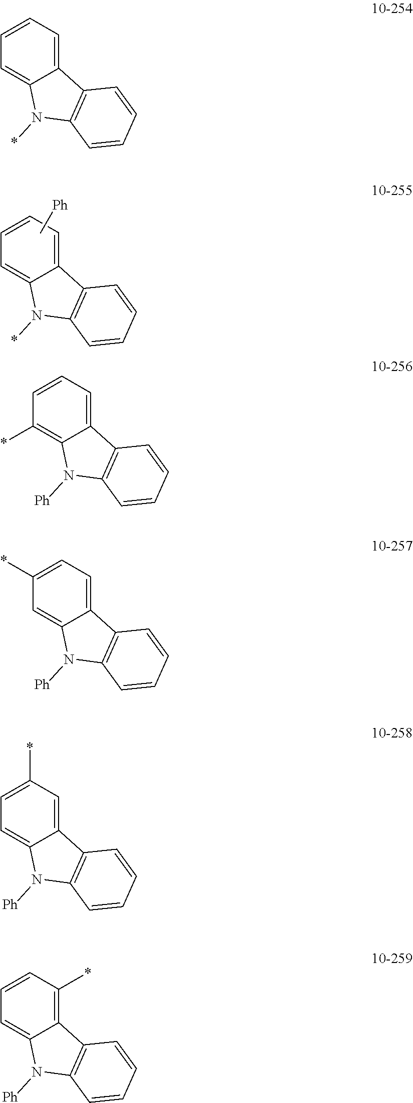

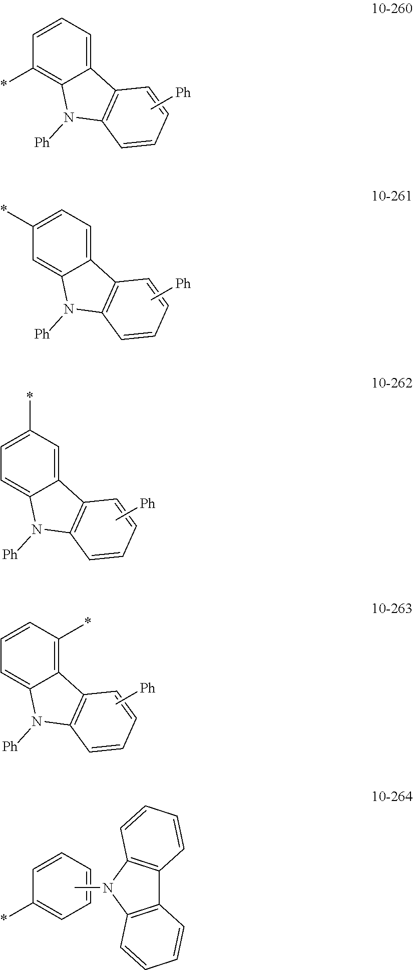

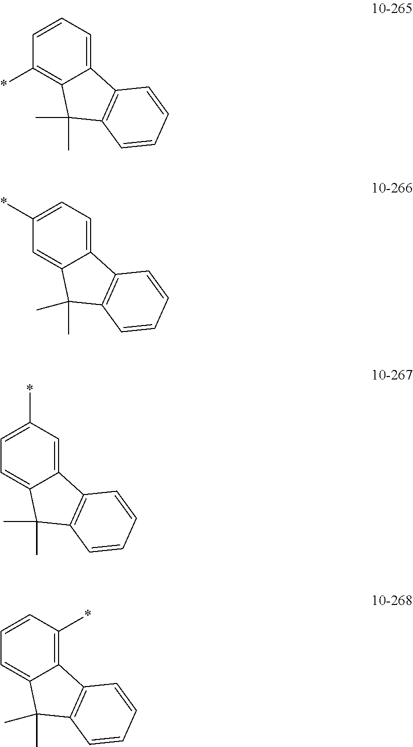 [SMILES: *c1cccc2c1C(C)(C)c1ccccc1-2 ; *c1ccc2c(c1)C(C)(C)c1ccccc1-2 ; *c1ccc2c(c1)-c1ccccc1C2(C)C ; *c1cccc2c1-c1ccccc1C2(C)C]

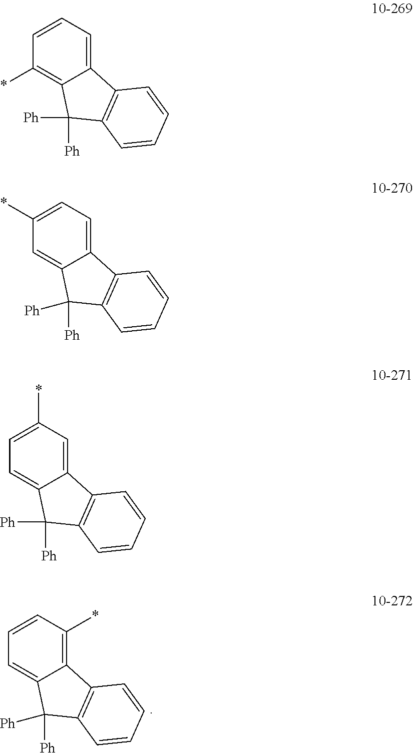 [SMILES: *c1cccc2c1C(c1ccccc1)(c1ccccc1)c1ccccc1-2 ; *c1ccc2c(c1)C(c1ccccc1)(c1ccccc1)c1ccccc1-2 ; *c1ccc2c(c1)-c1ccccc1C2(c1ccccc1)c1ccccc1 ; *c1cccc2c1-c1ccccc1C2(c1ccccc1)c1ccccc1]

In Formulae 10-12 to 10-23, 10-38 to 10-130, and 10-238 to 10-272,
“Ph” represents a phenyl group, and
* indicates a binding site to a neighboring atom.

In an embodiment, in Formulae 2-1 to 2-3, R₂₃ to R₃₁ may each independently be: hydrogen, deuterium, —F, —Cl, —Br, —I, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, —SF₅, a C₁-C₂₀ alkyl group, a C₁-C₂₀ alkenyl group, a C₁-C₂₀ alkoxy group, or a C₁-C₂₀ alkylthio group;
a C₁-C₂₀ alkyl group, a C₁-C₂₀ alkenyl group, a C₁-C₂₀ alkoxy group, or a C₁-C₂₀ alkylthio group, each substituted with deuterium, —F, —Cl, —Br, —I, —CD₃, —CD₂H, —CDH₂, —CF₃, —CF₂H, —CFH₂, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a C₁-C₁₀ alkyl group, a cyclopentyl group, a cyclohexyl group, a cycloheptyl group, a cyclooctyl group, an adamantanyl group, a norbornanyl group, a norbornenyl group, a cyclopentenyl group, a cyclohexenyl group, a cycloheptenyl group, a bicyclo[1.1.1]pentyl group, a bicyclo[2.1.1]hexyl group, a bicyclo[2.2.1]heptyl group, a bicyclo[2.2.2]octyl group, a (C₁-C₂₀ alkyl)cyclopentyl group, a (C₁-C₂₀ alkyl)cyclohexyl group, a (C₁-C₂₀ alkyl)cycloheptyl group, a (C₁-C₂₀ alkyl)cyclooctyl group, a (C₁-C₂₀ alkyl)adamantanyl group, a (C₁-C₂₀ alkyl)norbornanyl group, a (C₁-C₂₀ alkyl)norbornenyl group, a (C₁-C₂₀ alkyl)cyclopentenyl group, a (C₁-C₂₀ alkyl)cyclohexenyl group, a (C₁-C₂₀ alkyl)cycloheptenyl group, a (C₁-C₂₀ alkyl)bicyclo[1.1.1]pentyl group, a (C₁-C₂₀ alkyl)bicyclo[2.1.1]hexyl group, a (C₁-C₂₀ alkyl)bicyclo[2.2.1]heptyl group, a (C₁-C₂₀ alkyl)bicyclo[2.2.2]octyl group, a phenyl group, a (C₁-C₂₀ alkyl)phenyl group, a biphenyl group, a terphenyl group, a naphthyl group, a pyridinyl group, a pyrimidinyl group, or any combination thereof;
a cyclopentyl group, a cyclohexyl group, a cycoheptyl group, a cyclooctyl group, an adamantanyl group, a norbornanyl group, a norbornenyl group, a cyclopentenyl group, a cyclohexenyl group, a cycloheptenyl group, a bicyclo[1.1.1]pentyl group, a bicyclo[2.1.1]hexyl group, a bicyclo[2.2.1]heptyl group, a bicyclo[2.2.2]octyl group, a phenyl group, a (C₁-C₂₀ alkyl)phenyl group, a biphenyl group, a terphenyl group, a naphthyl group, a fluorenyl group, a phenanthrenyl group, an anthracenyl group, a fluoranthenyl group, a triphenylenyl group, a pyrenyl group, a chrysenyl group, a pyrrolyl group, a thiophenyl group, a furanyl group, an imidazolyl group, a pyrazolyl group, a thiazolyl group, an isothiazolyl group, an oxazolyl group, an isoxazolyl group, a pyridinyl group, a pyrazinyl group, a pyrimidinyl group, a pyridazinyl group, an isoindolyl group, an indolyl group, an indazolyl group, a purinyl group, a quinolinyl group, an isoquinolinyl group, a benzoquinolinyl group, a quinoxalinyl group, a quinazolinyl group, a cinnolinyl group, a carbazolyl group, a phenanthrolinyl group, a benzoimidazolyl group, a benzofuranyl group, a benzothiophenyl group, a benzoisothiazolyl group, a benzoxazolyl group, an isobenzoxazolyl group, a triazolyl group, a tetrazolyl group, an oxadiazolyl group, a triazinyl group, a dibenzofuranyl group, a dibenzothiophenyl group, a benzocarbazolyl group, a dibenzocarbazolyl group, an imidazopyridinyl group, an imidazopyrimidinyl group, an azacarbazolyl group, an azadibenzofuranyl group, or an azadibenzothiophenyl group, each unsubstituted or substituted with deuterium, —F, —CI, —Br, —I, —CD₃, —CD₂H, —CDH₂, —CF₃, —CF₂H, —CFH₂, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a C₁-C₂₀ alkyl group, a deuterated C₂-C₂₀ alkyl group, a fluorinated C₁-C₂₀ alkyl group, a C₁-C₂₀ alkoxy group, a cyclopentyl group, a cyclohexyl group, a cycloheptyl group, a cyclooctyl group, an adamantanyl group, a norbornanyl group, a norbornenyl group, a cyclopentenyl group, a cyclohexenyl group, a cycloheptenyl group, a bicyclo[1.1.1]pentyl group, a bicyclo[2.1.1]hexyl group, a bicyclo[2.2.1]heptyl group, a bicyclo[2.2.2]octyl group, a (C₁-C₂₀ alkyl)cyclopentyl group, a (C₁-C₂₀ alkyl)cyclohexyl group, a (C₁-C₂₀ alkyl)cycloheptyl group, a (C₁-C₂₀ alkyl)cyclooctyl group, a (C₁-C₂₀ alkyl)adamantanyl group, a (C₁-C₂₀ alkyl)norbornanyl group, a (C₁-C₂₀ alkyl)norbornenyl group, a (C₁-C₂₀ alkyl)cyclopentenyl group, a (C₁-C₂₀ alkyl)cyclohexenyl group, a (C₁-C₂₀ alkyl)cycloheptenyl group, a (C₁-C₂₀ alkyl)bicyclo[1.1.1]pentyl group, a (C₁-C₂₀ alkyl)bicyclo[2.1.1]hexyl group, a (C₁-C₂₀ alkyl)bicyclo[2.2.1]heptyl group, a (C₁-C₂₀ alkyl)bicyclo[2.2.2]octyl group, a phenyl group, a (C₁-C₂₀ alkyl)phenyl group, a biphenyl group, a terphenyl group, a naphthyl group, a fluorenyl group, a phenanthrenyl group, an anthracenyl group, a fluoranthenyl group, a triphenylenyl group, a pyrenyl group, a chrysenyl group, a pyrrolyl group, a thiophenyl group, a furanyl group, an imidazolyl group, a pyrazolyl group, a thiazolyl group, an isothiazolyl group, an oxazolyl group, an isoxazolyl group, a pyridinyl group, a pyrazinyl group, a pyrimidinyl group, a pyridazinyl group, an isoindolyl group, an indolyl group, an indazolyl group, a purinyl group, a quinolinyl group, an isoquinolinyl group, a benzoquinolinyl group, a quinoxalinyl group, a quinazolinyl group, a cinnolinyl group, a carbazolyl group, a phenanthrolinyl group, a benzoimidazolyl group, a benzofuranyl group, a benzothiophenyl group, a benzoisothiazolyl group, a benzoxazolyl group, an isobenzoxazolyl group, a triazolyl group, a tetrazolyl group, an oxadiazolyl group, a triazinyl group, a dibenzofuranyl group, a dibenzothiophenyl group, a benzocarbazolyl group, a dibenzocarbazolyl group, an imidazopyridinyl group, an imidazopyrimidinyl group, an azacarbazolyl group, an azadibenzofuranyl group, an azadibenzothiophenyl group, or any combination thereof; or
—Si(Q₁)(Q₂)(Q₃), —C(Q₁)(Q₂)(Q₃), —B(Q₁)(Q₂), or —N(Q₁)(Q₂), and Q₁ to Q₃ may each independently be:
deuterium, —F, —CH₃, —CD₃, —CD₂H, —CDH₂, —CH₂CH₃, —CH₂CD₃, —CH₂CD₂H, —CH₂CDH₂, —CHDCH₃, —CHDCD₂H, —CHDCDH₂, —CHDCD₃, —CD₂CD₃, —CD₂CD₂H, —CD₂CDH₂, —CF₃, —CF₂H, —CFH₂, —CH₂CF₃, —CH₂CF₂H, —CH₂CFH₂, —CHFCH₃, —CHFCF₂H, —CHFCFH₂, —CHFCF₃, —CF₂CF₃, —CF₂CF₂H, or —CF₂CFH₂; or
an n-propyl group, an iso-propyl group, an n-butyl group, a sec-butyl group, an isobutyl group, a tert-butyl group, an n-pentyl group, a tert-pentyl group, a neopentyl group, an isopentyl group, a sec-pentyl group, a 3-pentyl group, a sec-isopentyl group, a phenyl group, a biphenyl group, or a naphthyl group, each unsubstituted or substituted with deuterium, —F, a C₁-C₁₀ alkyl group, a phenyl group, or any combination thereof.

In one or more embodiments, in Formulae 2-1 to 2-3, R₂₃ to R₃₁ may each independently be hydrogen, deuterium, —F, —CH₃, —CD₃, —CD₂H, —CDH₂, —CF₃, —CF₂H, —CFH₂, a C₂-C₁₀ alkenyl group, a C₁-C₁₀ alkoxy group, a C₁-C₁₀ alkylthio group, a group represented by one of Formulae 9-1 to 9-39, a group represented by one of Formulae 9-1 to 9-39 in which at least one hydrogen is substituted with deuterium, a group represented by one of Formulae 9-1 to 9-39 in which at least one hydrogen is substituted with —F, a group represented by one of Formulae 9-201 to 9-227, a group represented by one of Formulae 9-201 to 9-227 in which at least one hydrogen is substituted with deuterium, a group represented by one of Formulae 9-201 to 9-227 in which at least one hydrogen is substituted with —F, a group represented by one of Formulae 10-1 to 10-129, a group represented by one of Formulae 10-1 to 10-129 in which at least one hydrogen is substituted with deuterium, a group represented by one of Formulae 10-1 to 10-129 in which at least one hydrogen is substituted with —F, a group represented by one of Formulae 10-201 to 10-350, a group represented by one of Formulae 10-201 to 10-350 in which at least one hydrogen is substituted with deuterium, a group represented by one of Formulae 10-201 to 10-350 in which at least one hydrogen is substituted with —F, —Si(Q₃)(Q₄)(Q₅), or —C(Q₁)(Q₂)(Q₃), and
Q₁ to Q₃ may each independently be:
deuterium, —F, —CH₃, —CD₃, —CD₂H, —CDH₂, —CH₂CH₃, —CH₂CD₃, —CH₂CD₂H, —CH₂CDH₂, —CHDCH₃, —CHDCD₂H, —CHDCDH₂, —CHDCD₃, —CD₂CD₃, —CD₂CD₂H, —CD₂CDH₂, —CF₃, —CF₂H, —CFH₂, —CH₂CF₃, —CH₂CF₂H, —CH₂CFH₂, —CHFCH₃, —CHFCF₂H, —CHFCFH₂, —CHFCF₃, —CF₂CF₃, —CF₂CF₂H, or —CF₂CFH₂; or
an n-propyl group, an isopropyl group, an n-butyl group, a sec-butyl group, an isobutyl group, a tert-butyl group, an n-pentyl group, a tert-pentyl group, a neopentyl group, an isopentyl group, a sec-pentyl group, a 3-pentyl group, a sec-isopentyl group, a phenyl group, a biphenyl group, or a naphthyl group, each unsubstituted or substituted with deuterium, —F, a C₁-C₁₀ alkyl group, a phenyl group, or any combination thereof:

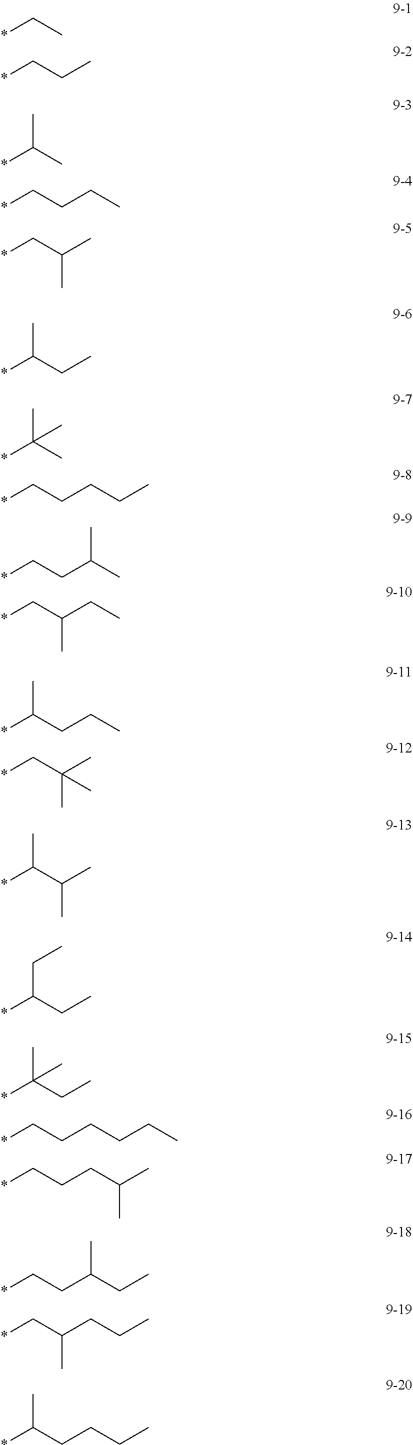

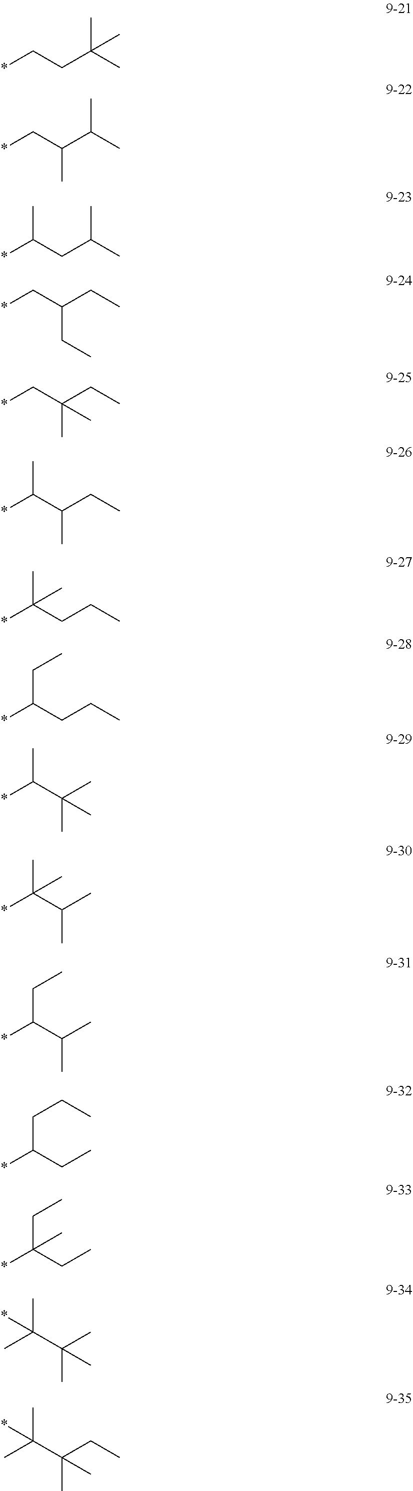

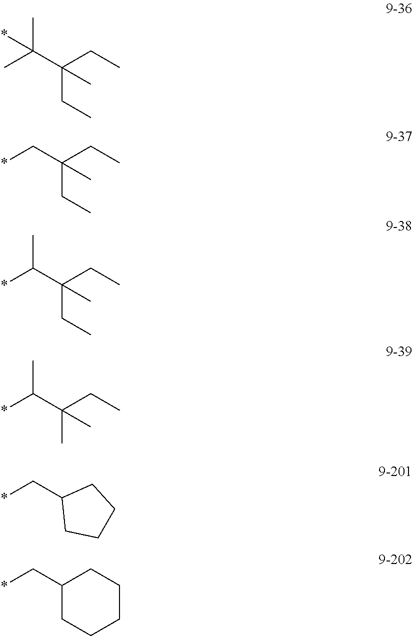

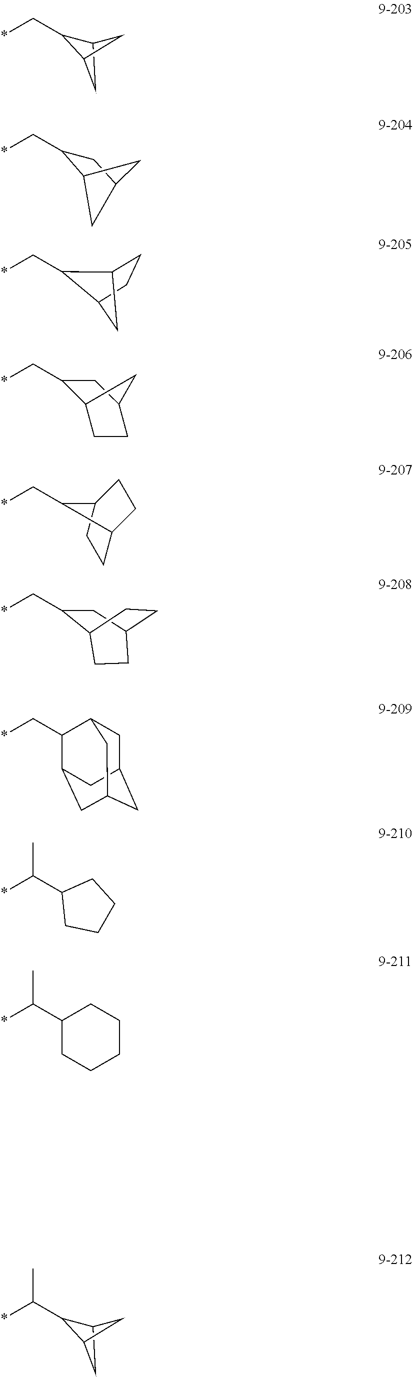

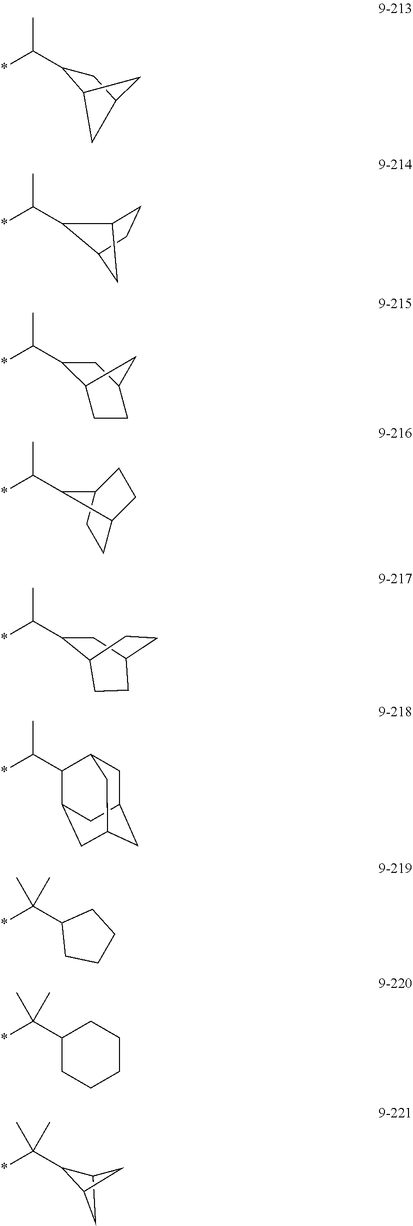

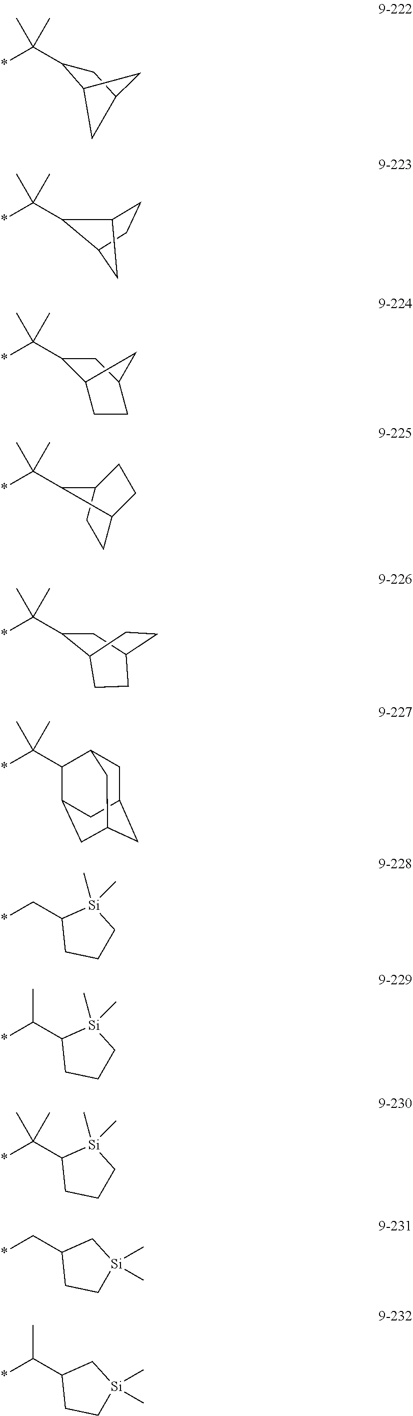

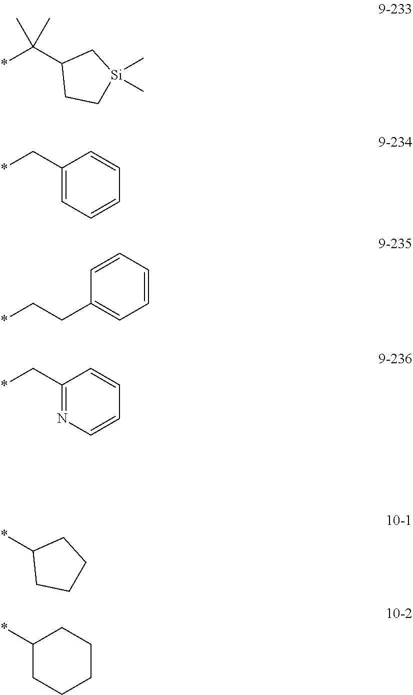

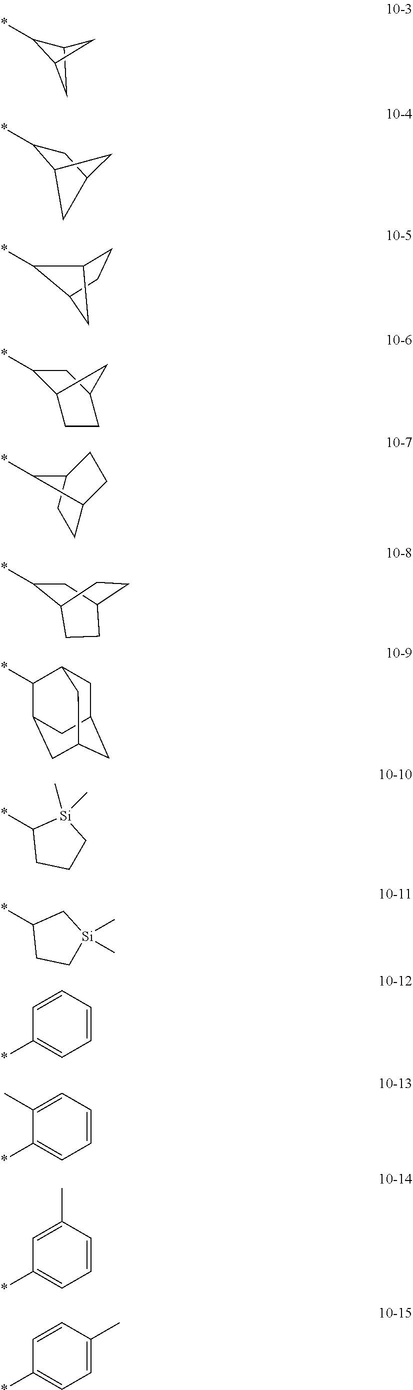

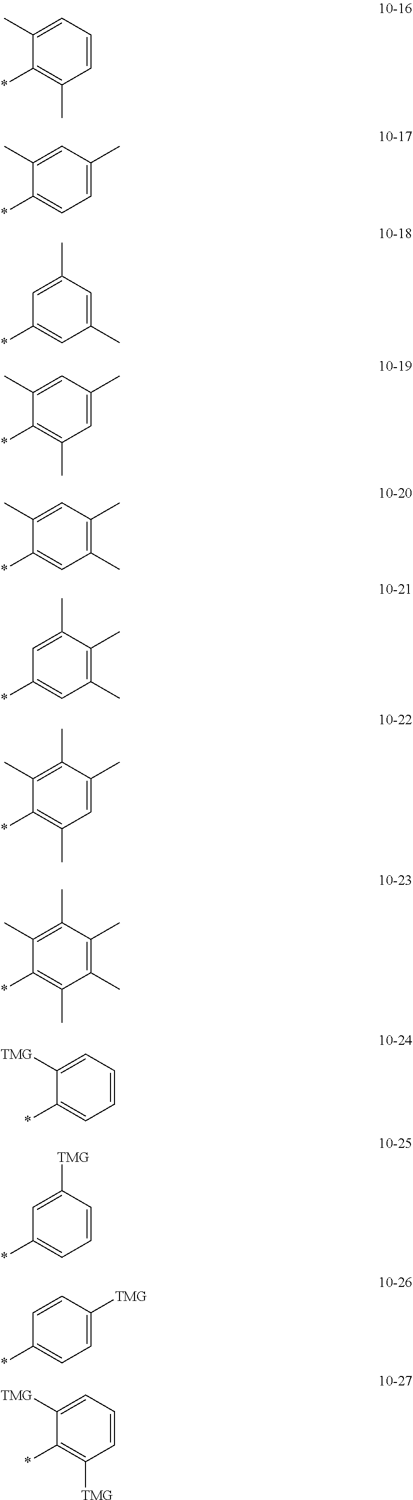

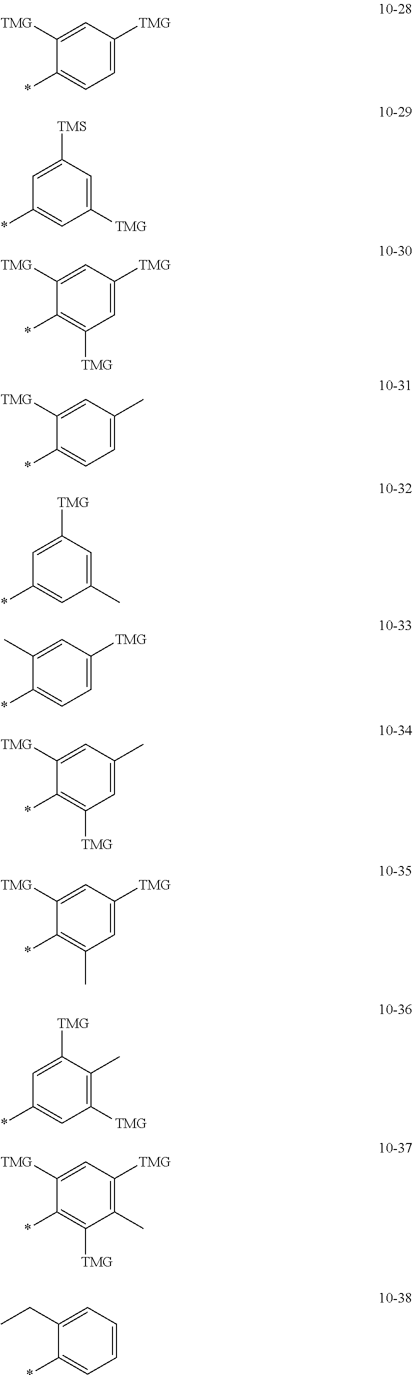

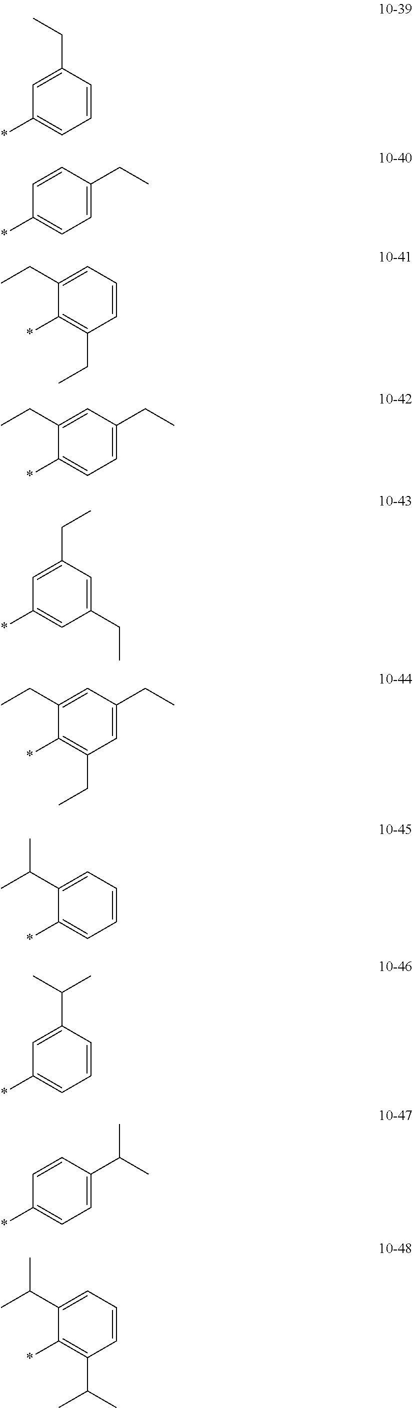

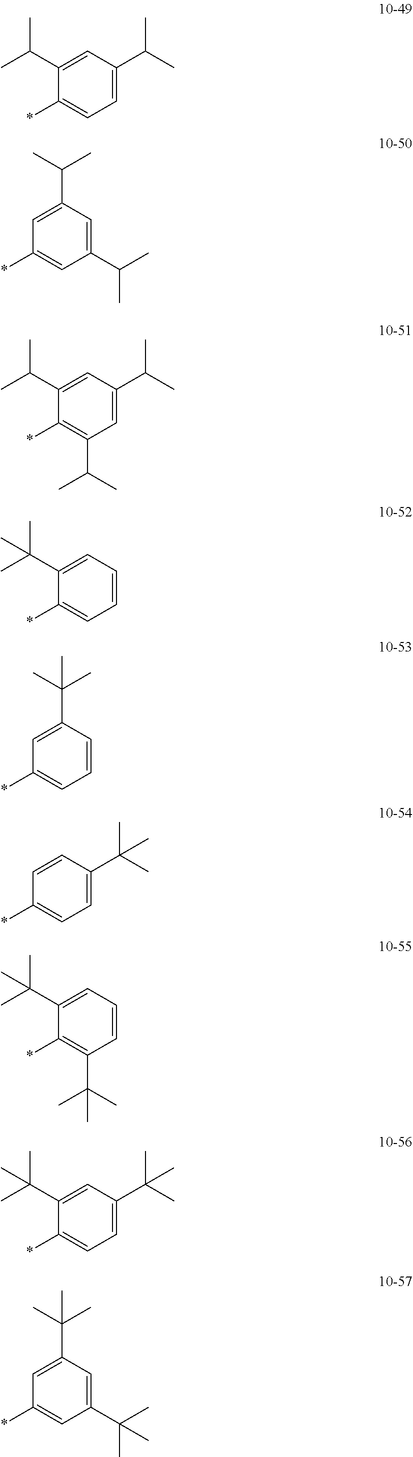

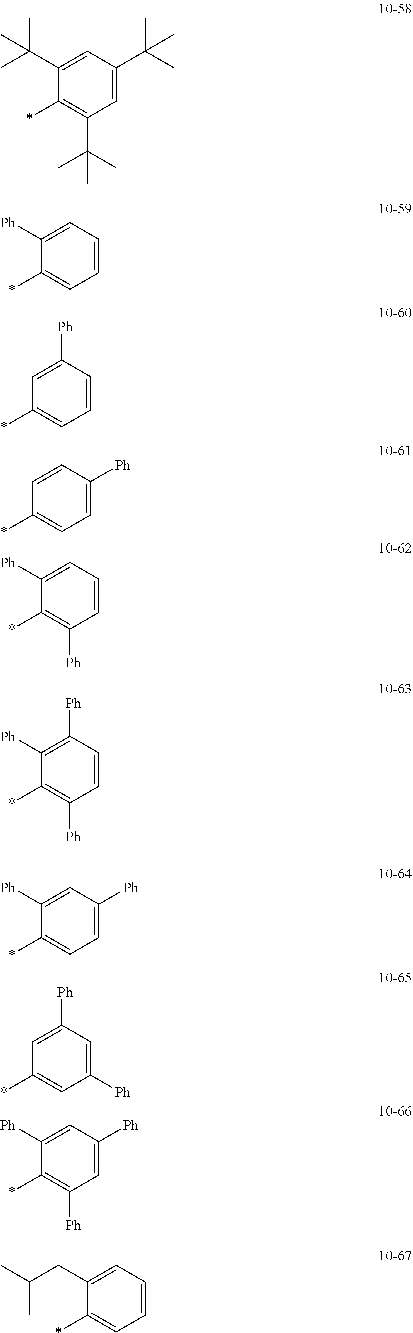

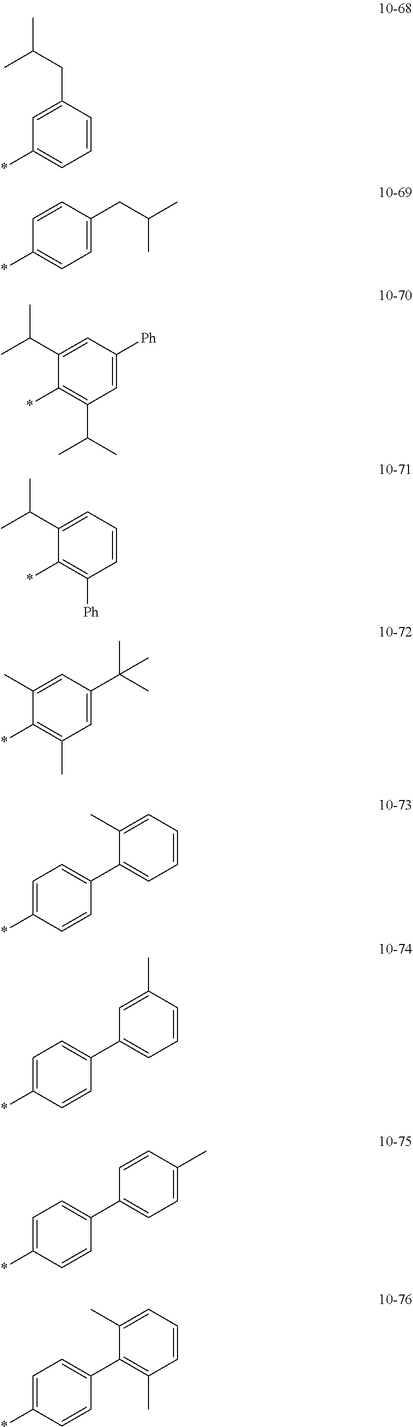

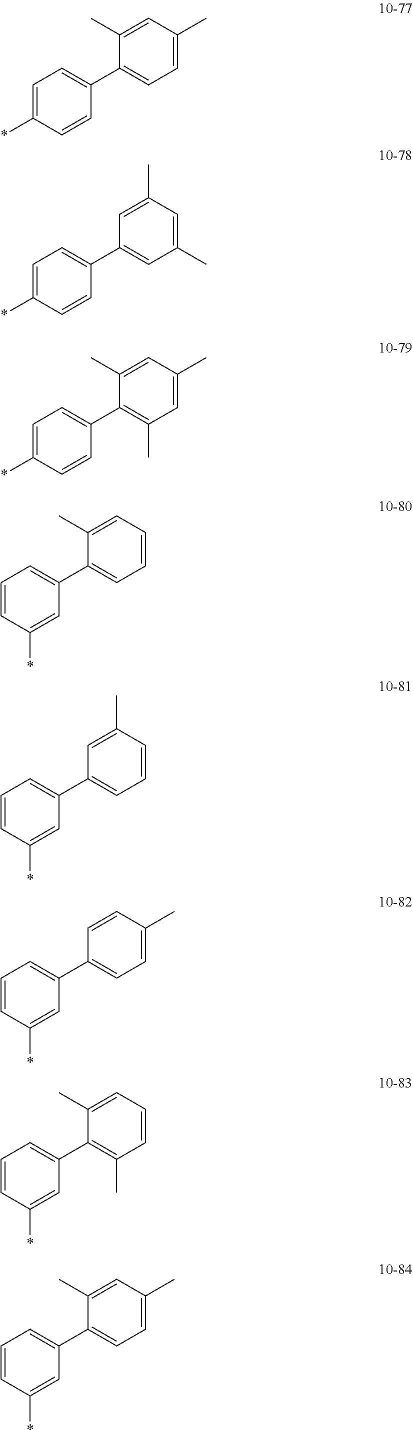

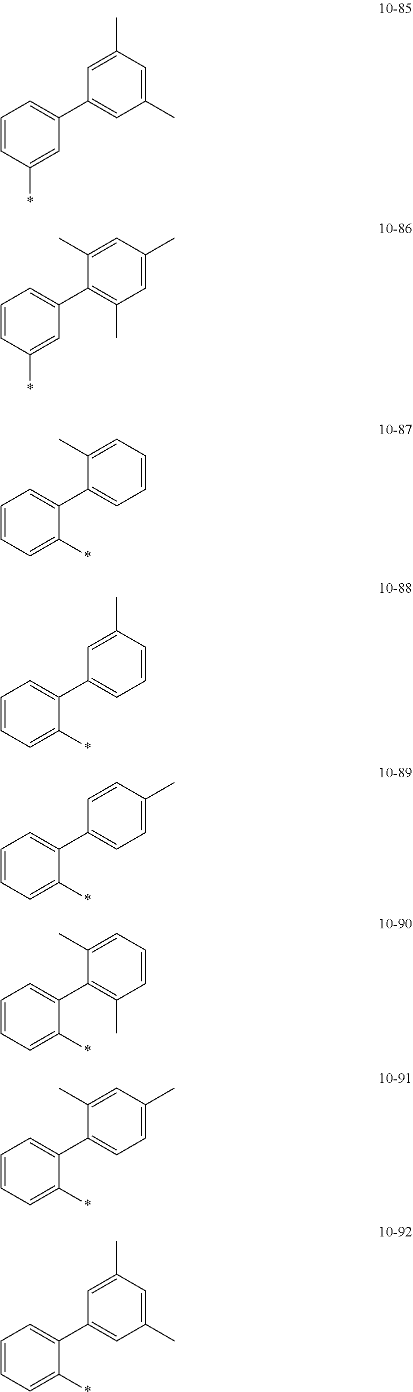

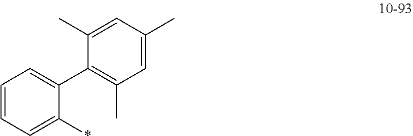 [SMILES: *c1ccccc1-c1c(C)cc(C)cc1C]

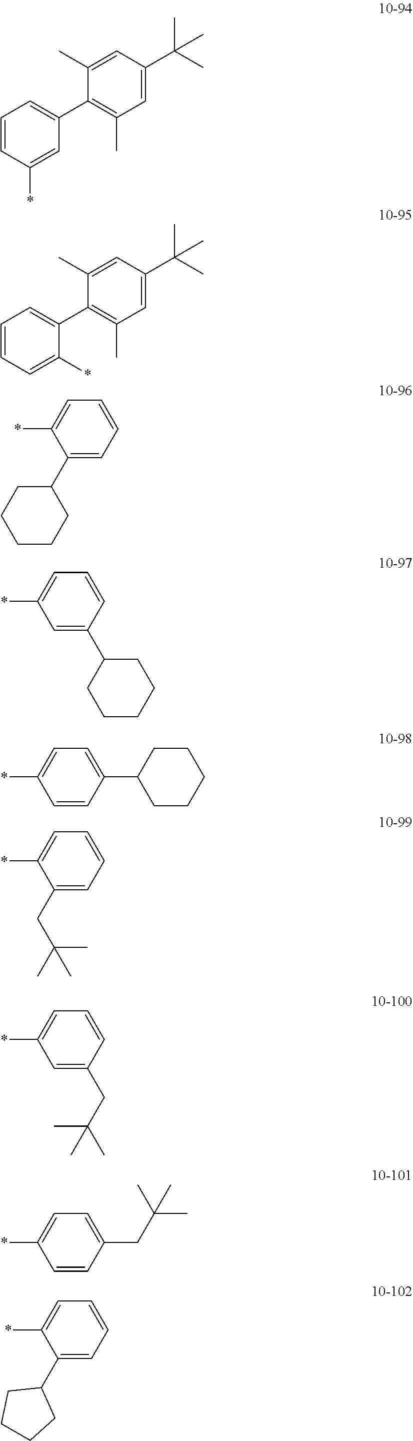

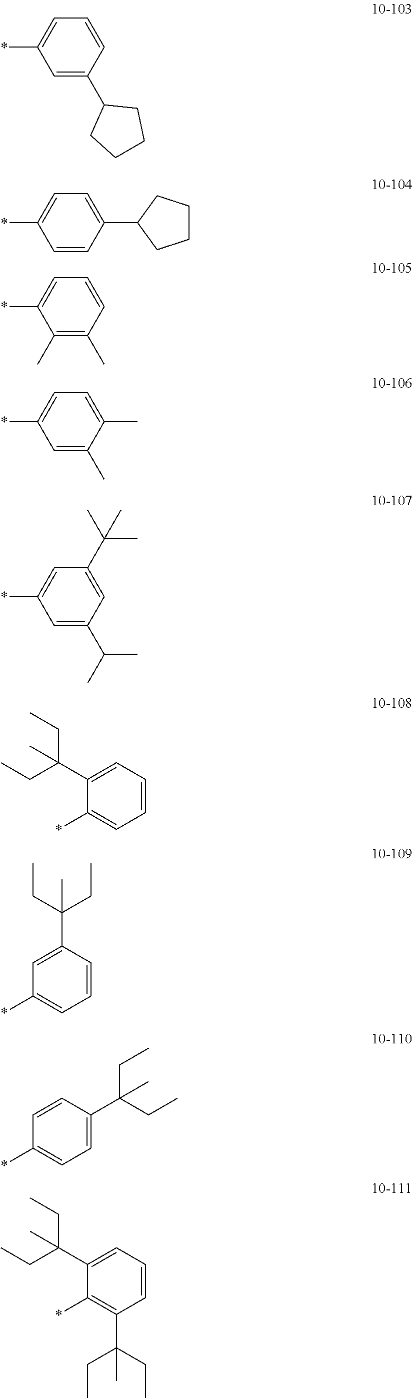

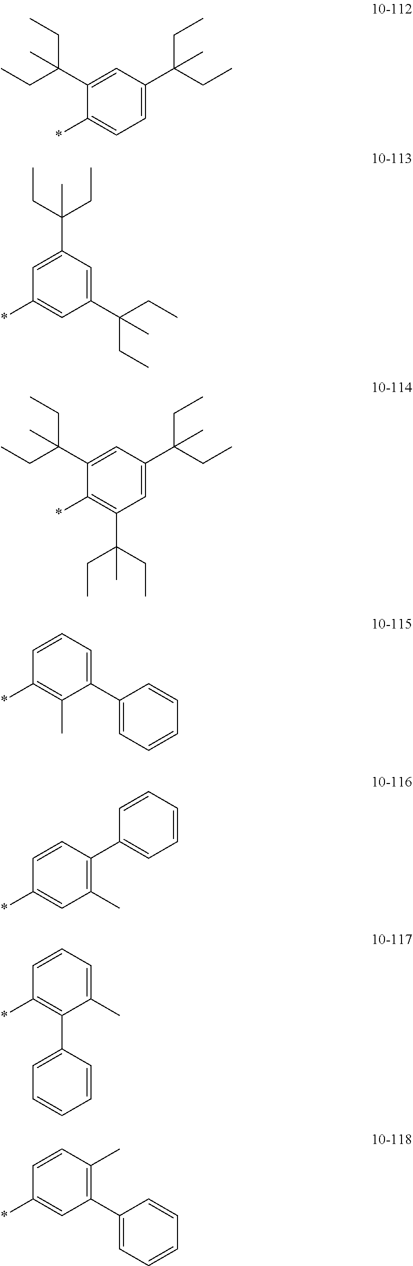

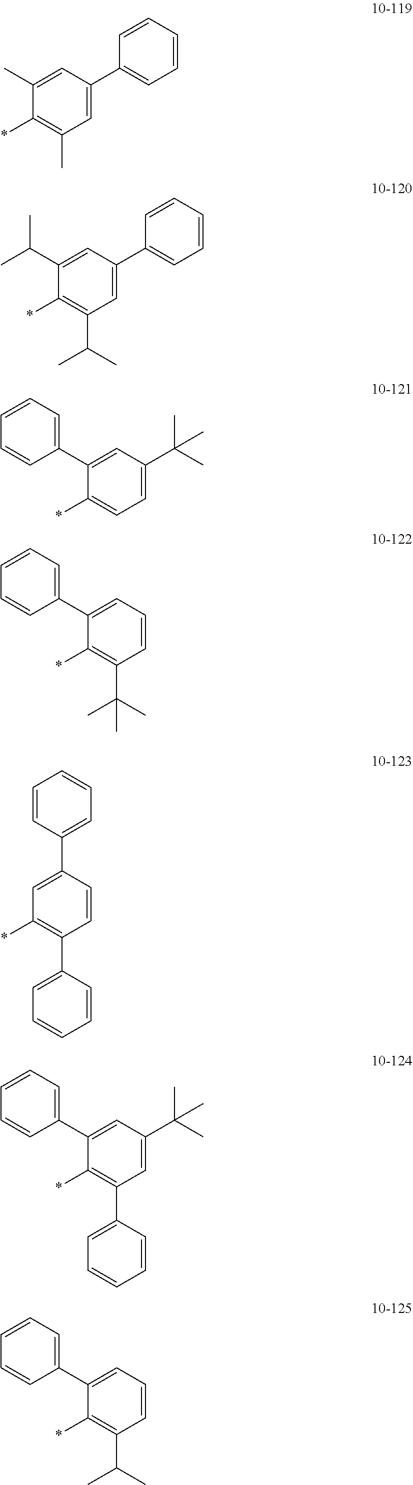

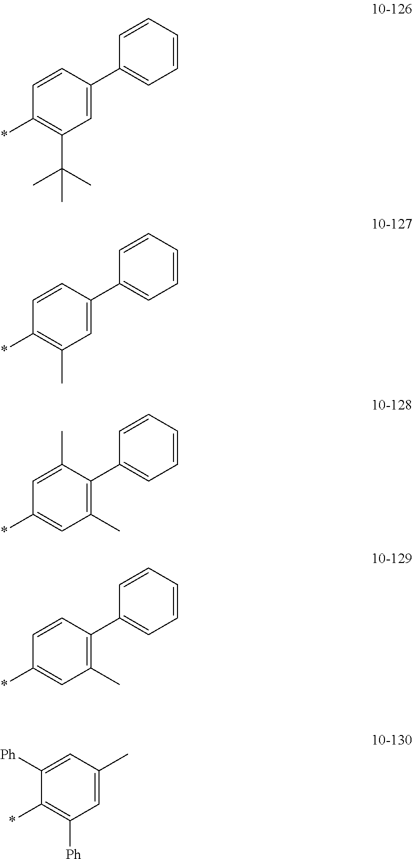 [SMILES: *c1ccc(-c2ccccc2)cc1C(C)(C)C ; *c1ccc(-c2ccccc2)cc1C ; *c1cc(C)c(-c2ccccc2)c(C)c1 ; *c1ccc(-c2ccccc2)c(C)c1 ; *c1c(-c2ccccc2)cc(C)cc1-c1ccccc1]

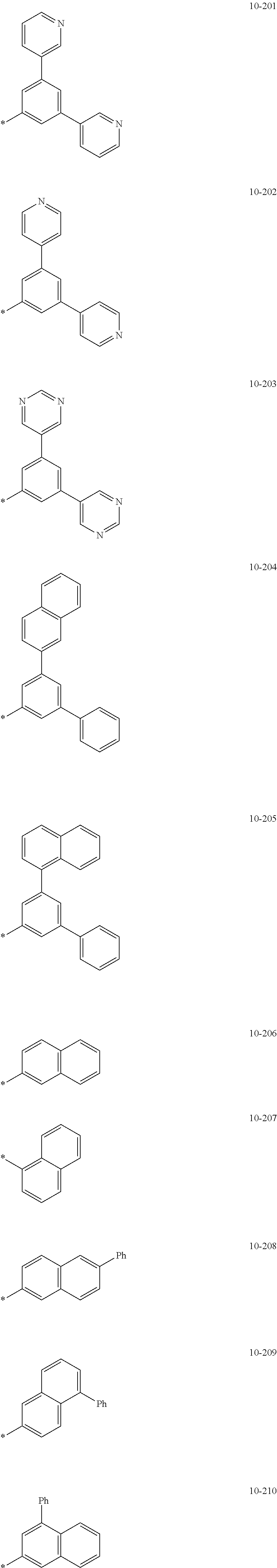

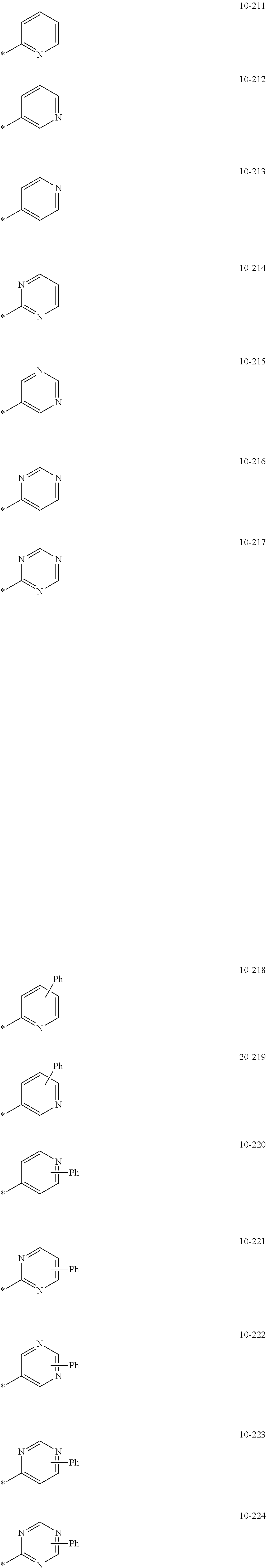

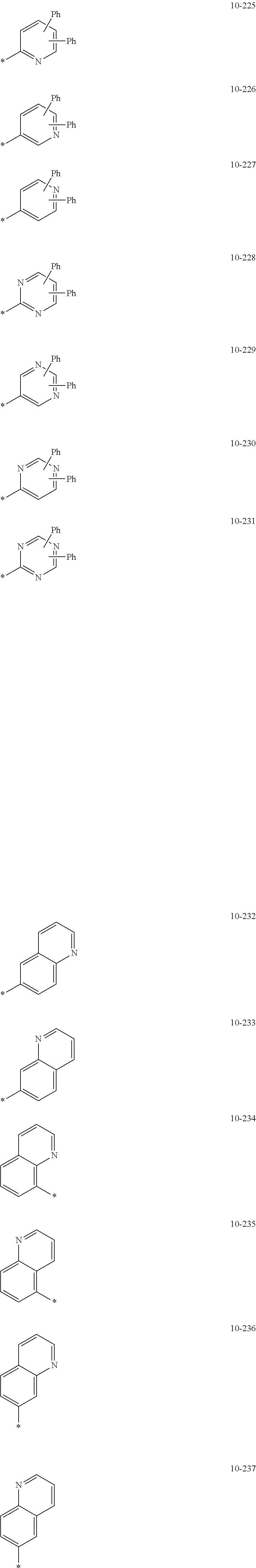

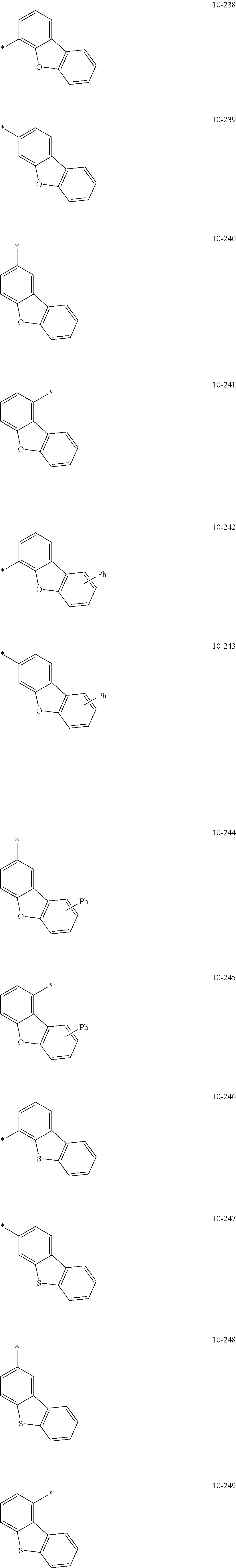

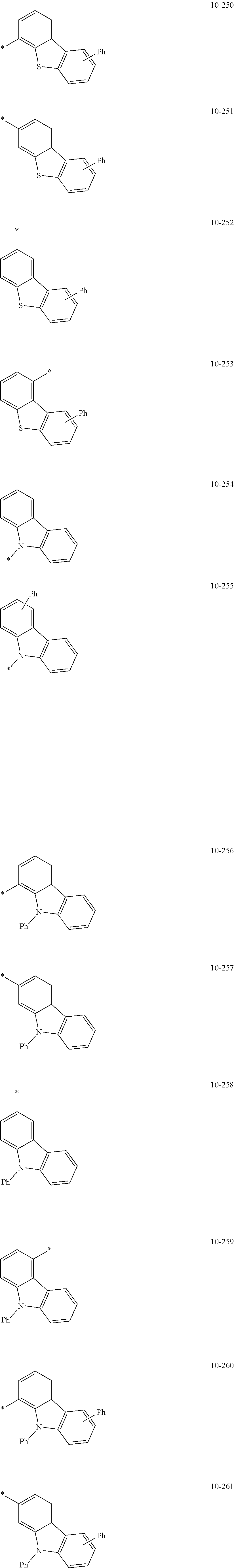

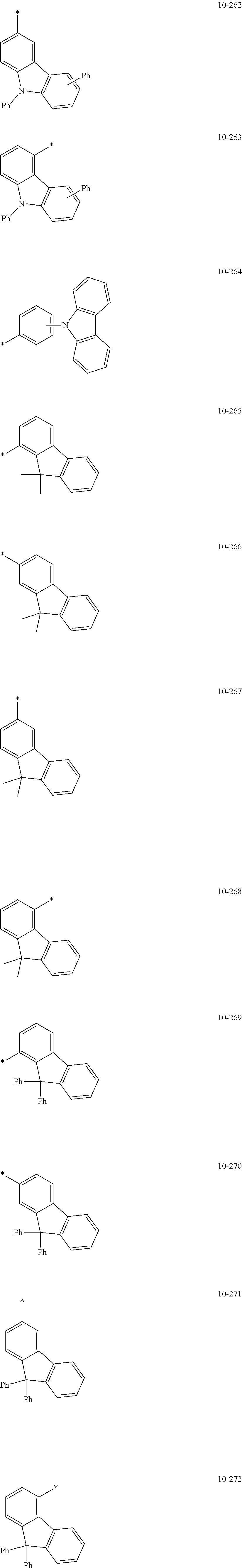

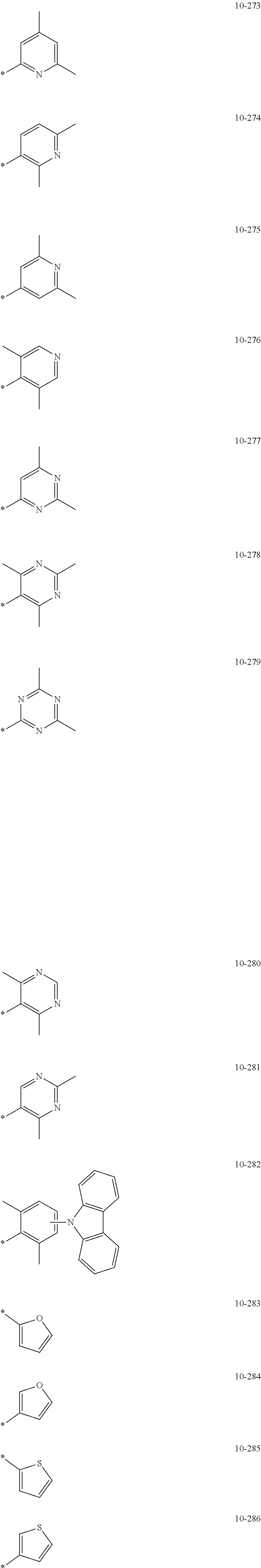

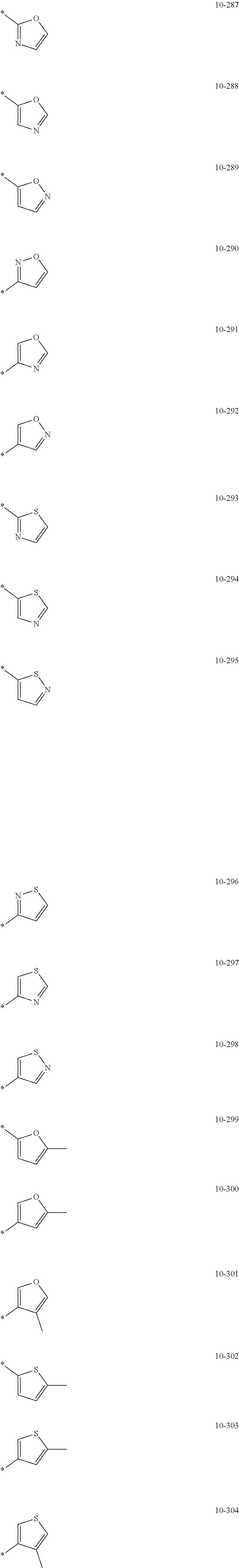

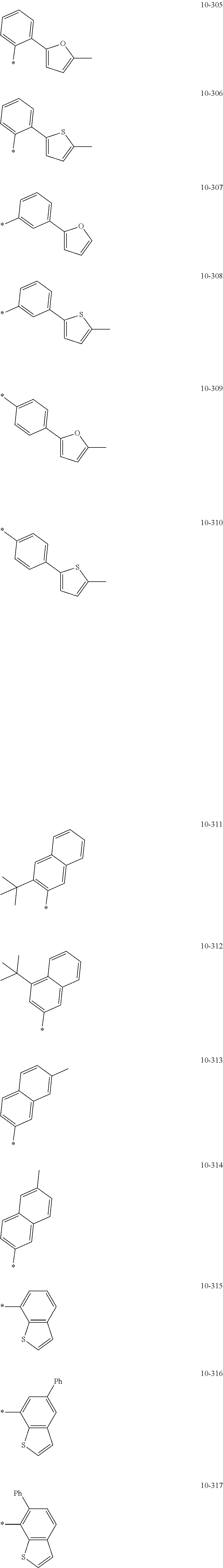

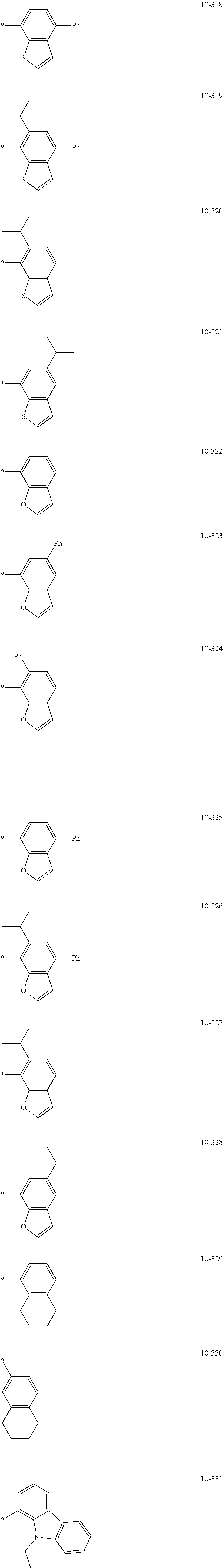

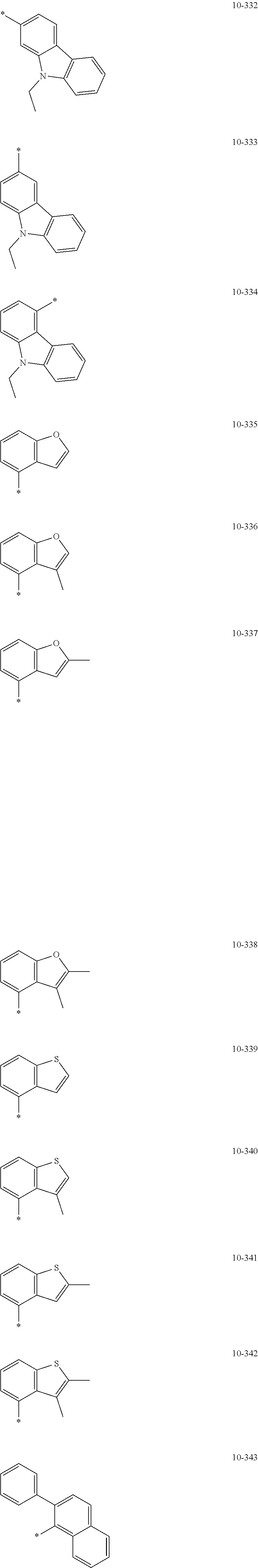

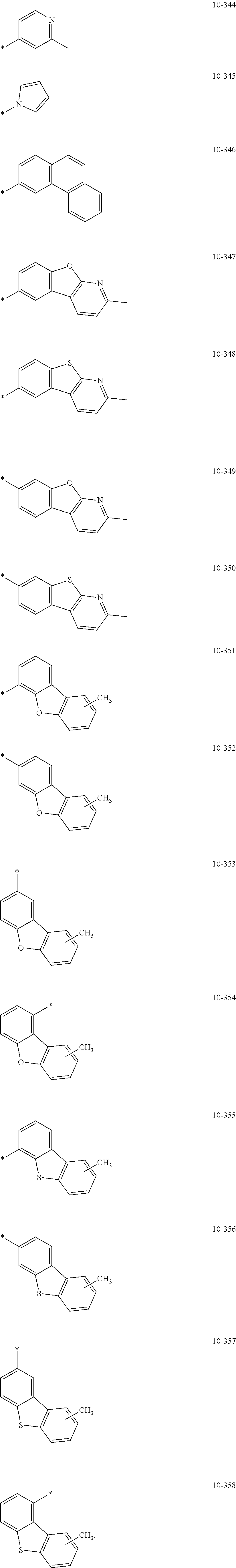

In Formulae 9-1 to 9-39, 9-201 to 9-236, 10-1 to 10-130, and 10-201 to 10-358, * indicates a binding site to an adjacent atom, “Ph” represents a phenyl group, “TMS” and “SiMe₃” each represent a trimethylsilyl group, and “TMG” and “GeMe₃” each represent a trimethylgermyl group.

The “group represented by Formulae 9-1 to 9-39 in which at least one hydrogen is substituted with deuterium” and the “group represented by Formulae 9-201 to 9-236 in which at least one hydrogen is substituted with deuterium” may each be, for example, a group represented by one of Formulae 9-501 to 9-514 and 9-601 to 9-636:

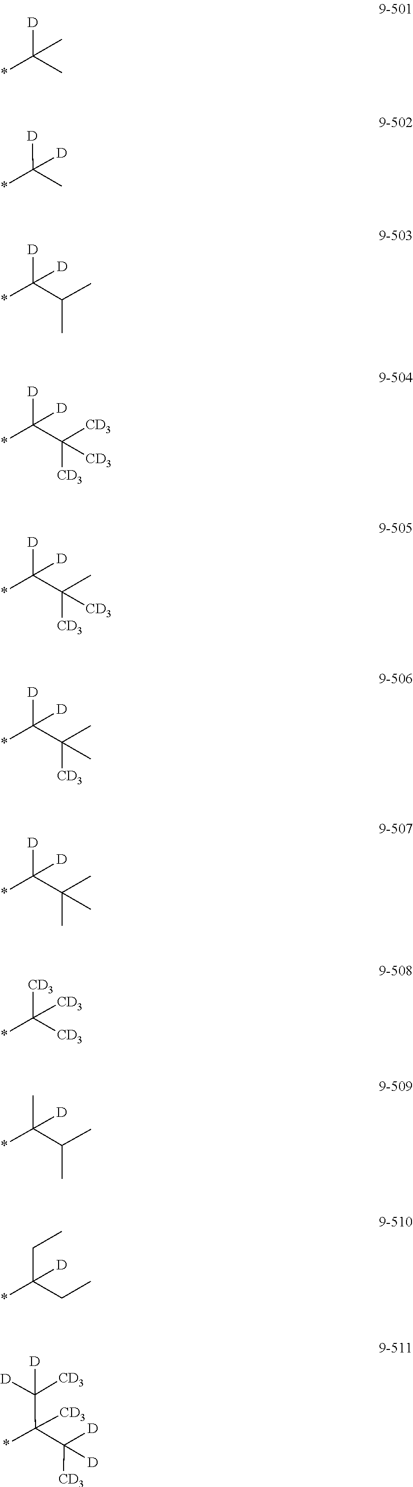

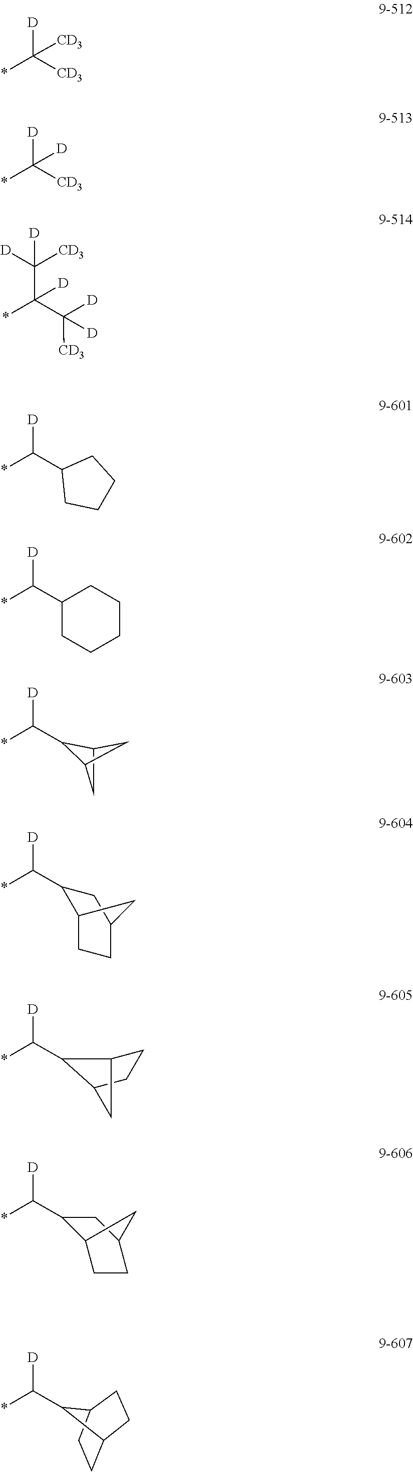

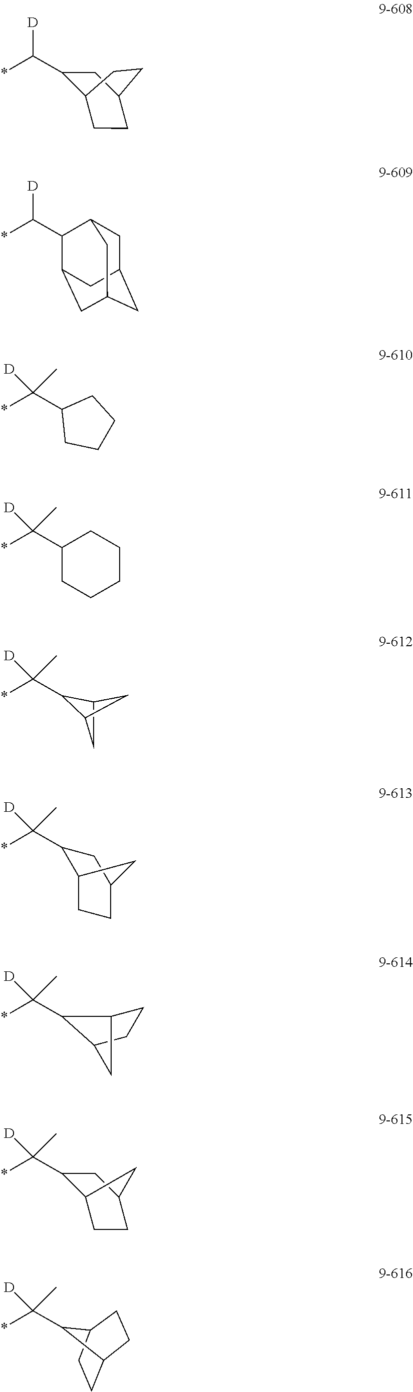

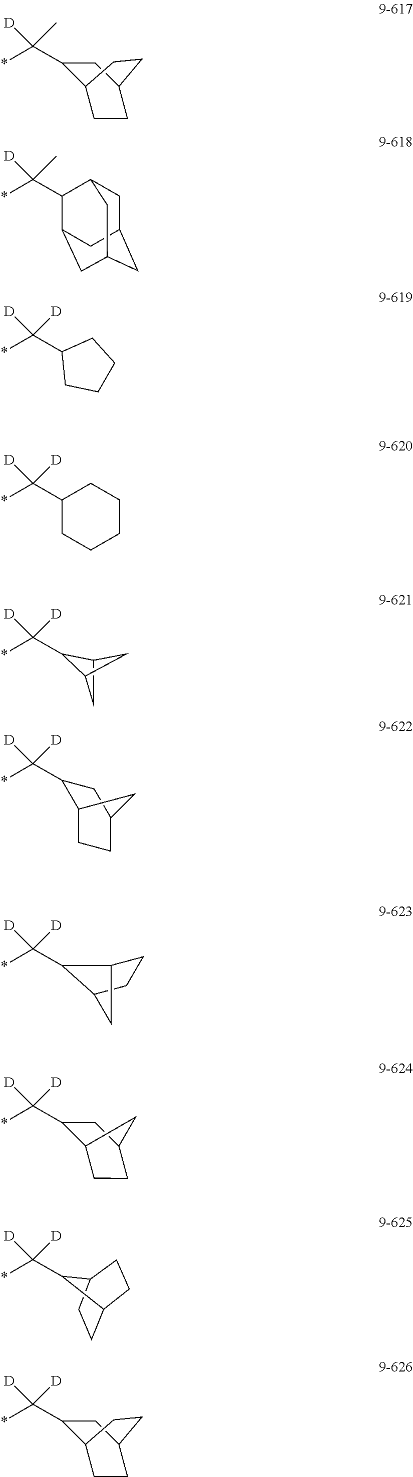

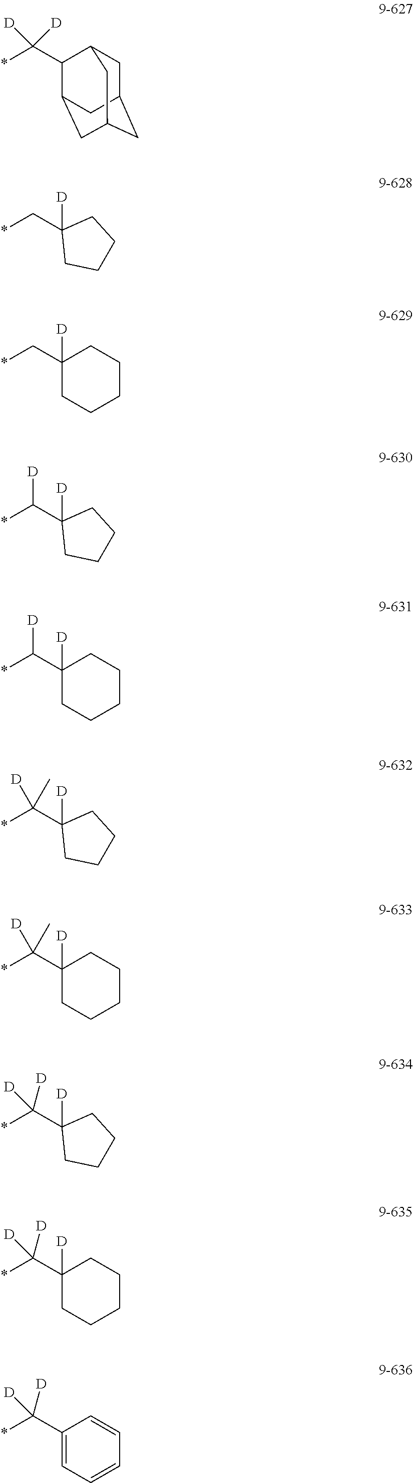

The “group represented by Formulae 9-1 to 9-39 in which at least one hydrogen is substituted with —F” and the “group represented by Formulae 9-201 to 9-236 in which at least one hydrogen is substituted with —F” may each be, for example, a group represented by one of Formulae 9-701 to 9-710:

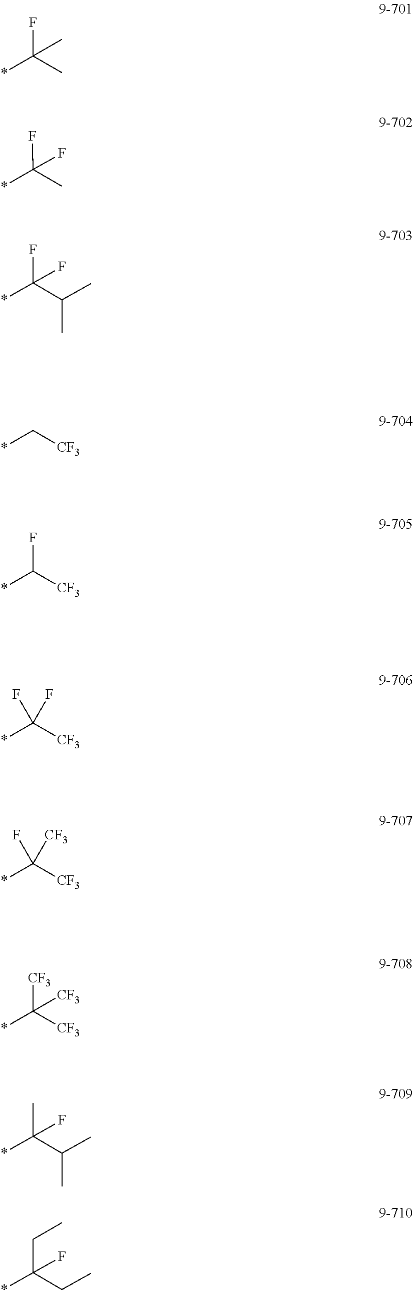

The “group represented by Formulae 10-1 to 10-130 in which at least one hydrogen is substituted with a deuterium” and the “group represented by Formulae 10-201 to 10-358 in which at least one hydrogen is substituted with deuterium” may each be, for example, a group represented by one of Formulae 10-501 to 576:

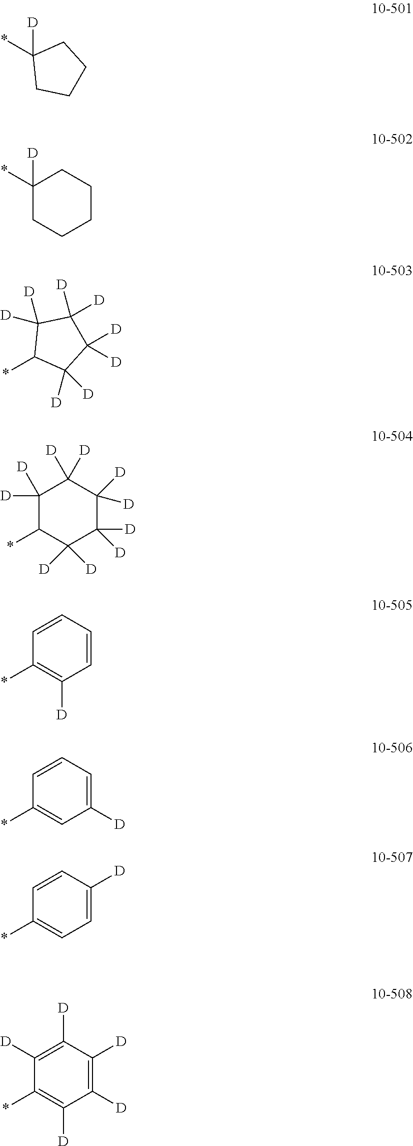

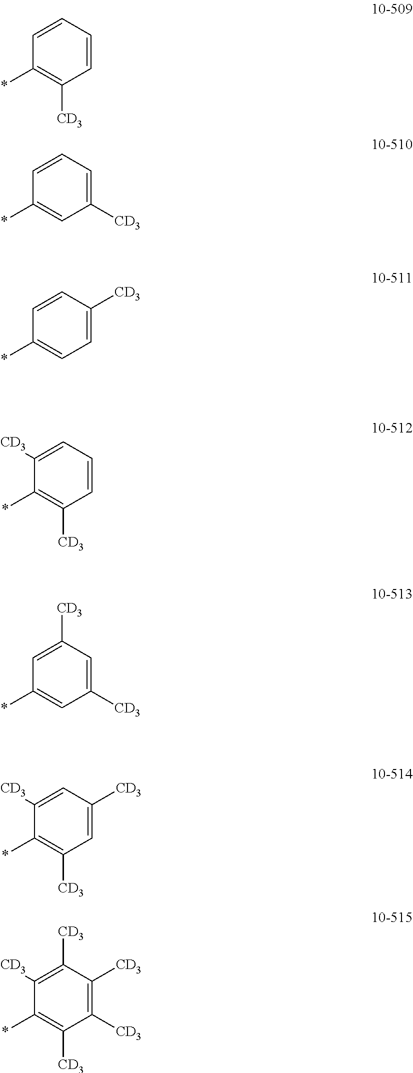

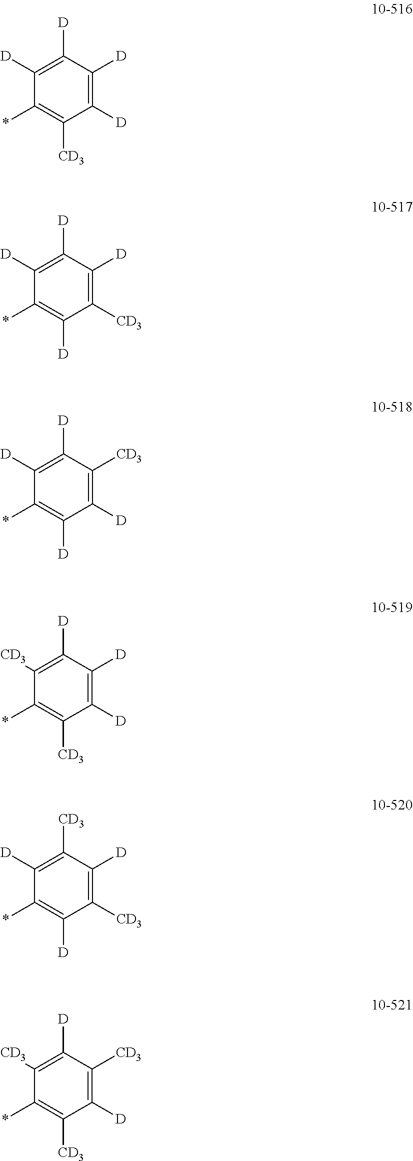 [SMILES: *c1c([2H])c([2H])c([2H])c([2H])c1C([2H])([2H])[2H] ; *c1c([2H])c([2H])c([2H])c(C([2H])([2H])[2H])c1[2H] ; *c1c([2H])c([2H])c(C([2H])([2H])[2H])c([2H])c1[2H] ; *c1c(C([2H])([2H])[2H])c([2H])c([2H])c([2H])c1C([2H])([2H])[2H] ; *c1c([2H])c(C([2H])([2H])[2H])c([2H])c(C([2H])([2H])[2H])c1[2H] ; *c1c(C([2H])([2H])[2H])c([2H])c(C([2H])([2H])[2H])c([2H])c1C([2H])([2H])[2H]]

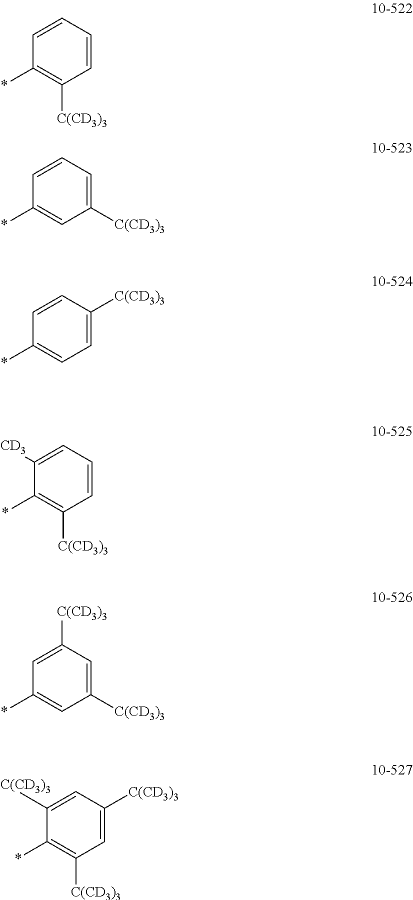

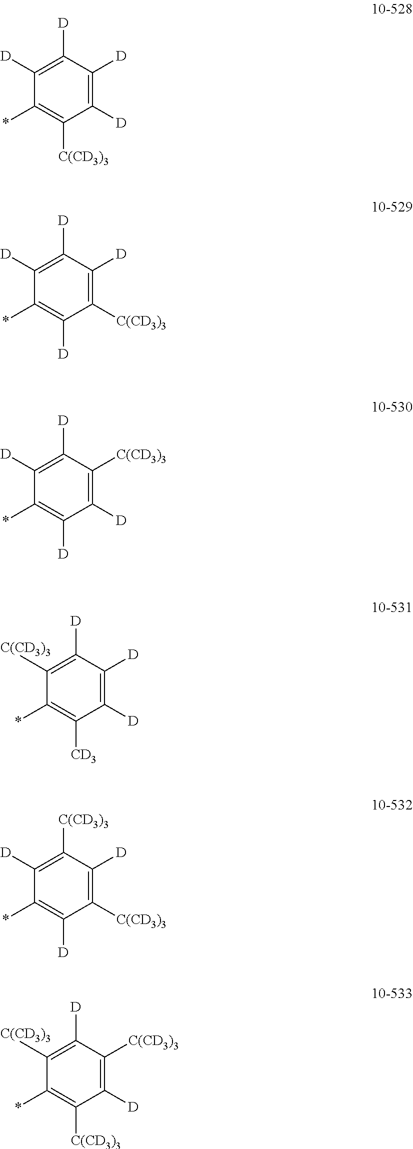

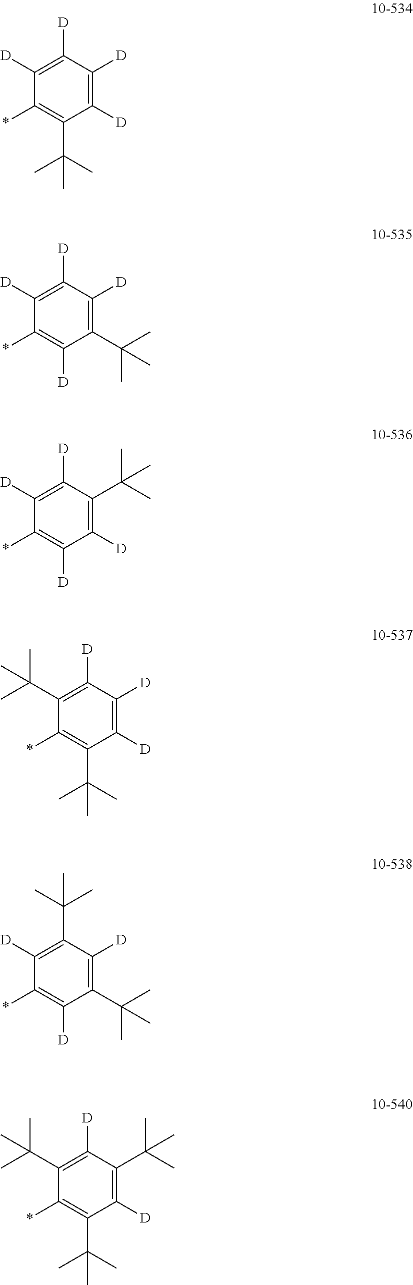

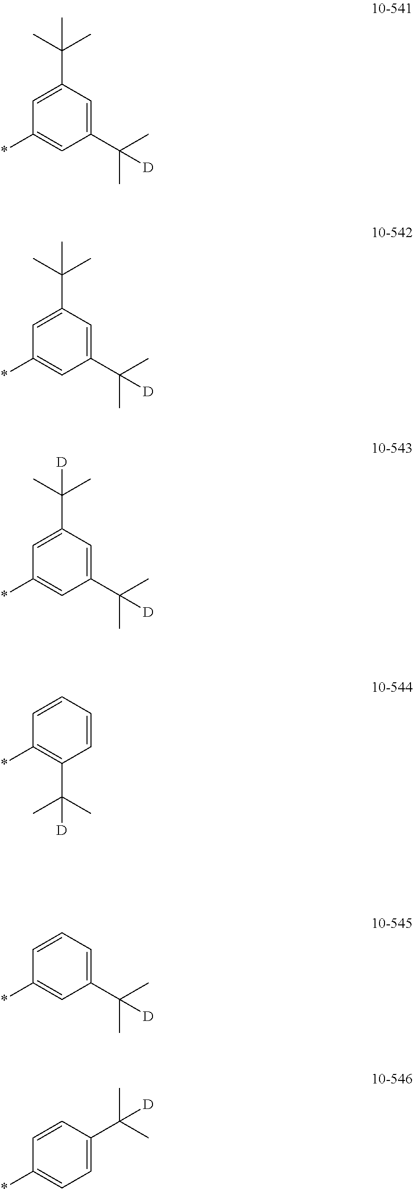 [SMILES: *c1cc(C([2H])(C)C)cc(C(C)(C)C)c1 ; *c1cc(C([2H])(C)C)cc(C(C)(C)C)c1 ; *c1cc(C([2H])(C)C)cc(C([2H])(C)C)c1 ; *c1ccccc1C([2H])(C)C ; *c1cccc(C([2H])(C)C)c1 ; *c1ccc(C([2H])(C)C)cc1]

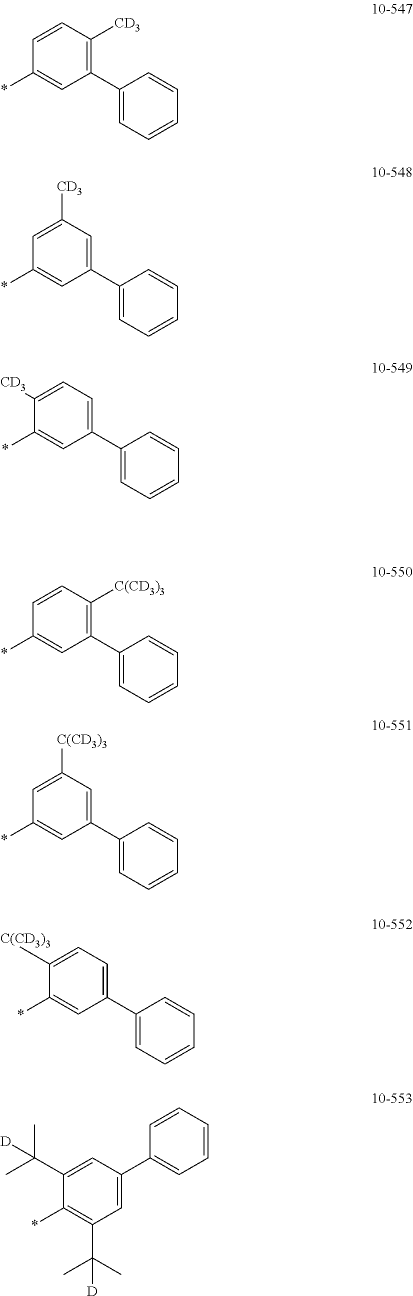

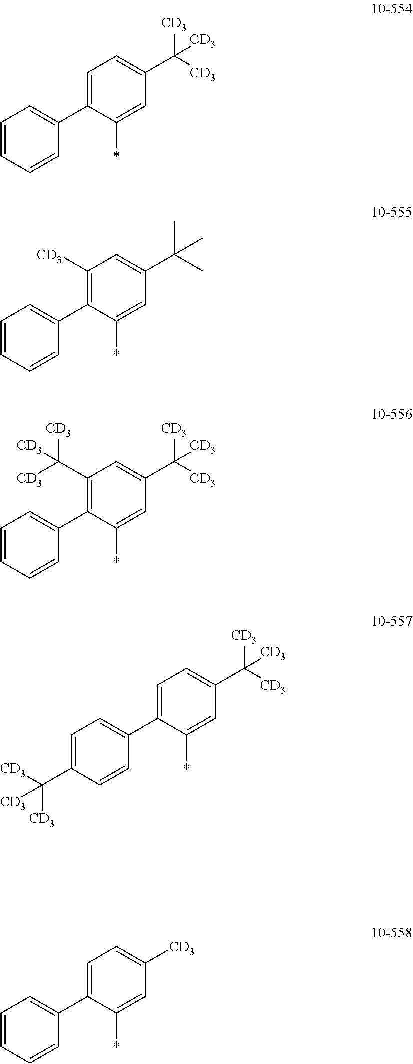 [SMILES: *c1cc(C(C([2H])([2H])[2H])(C([2H])([2H])[2H])C([2H])([2H])[2H])ccc1-c1ccccc1 ; *c1cc(C(C)(C)C)cc(C([2H])([2H])[2H])c1-c1ccccc1 ; *c1cc(C(C([2H])([2H])[2H])(C([2H])([2H])[2H])C([2H])([2H])[2H])cc(C(C([2H])([2H])[2H])(C([2H])([2H])[2H])C([2H])([2H])[2H])c1-c1ccccc1 ; *c1cc(C(C([2H])([2H])[2H])(C([2H])([2H])[2H])C([2H])([2H])[2H])ccc1-c1ccc(C(C([2H])([2H])[2H])(C([2H])([2H])[2H])C([2H])([2H])[2H])cc1 ; *c1cc(C([2H])([2H])[2H])ccc1-c1ccccc1]

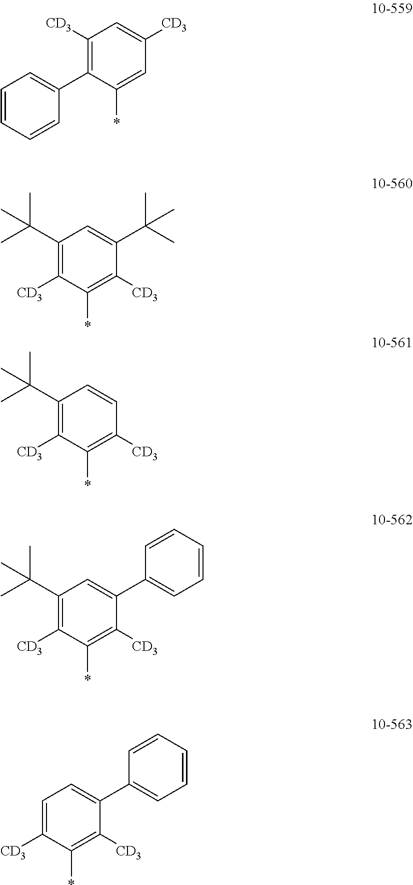

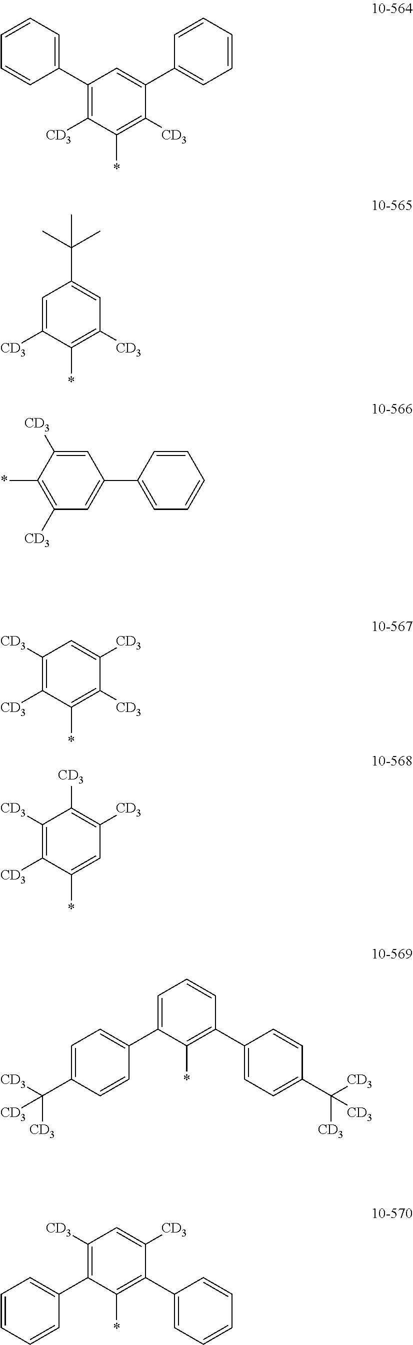

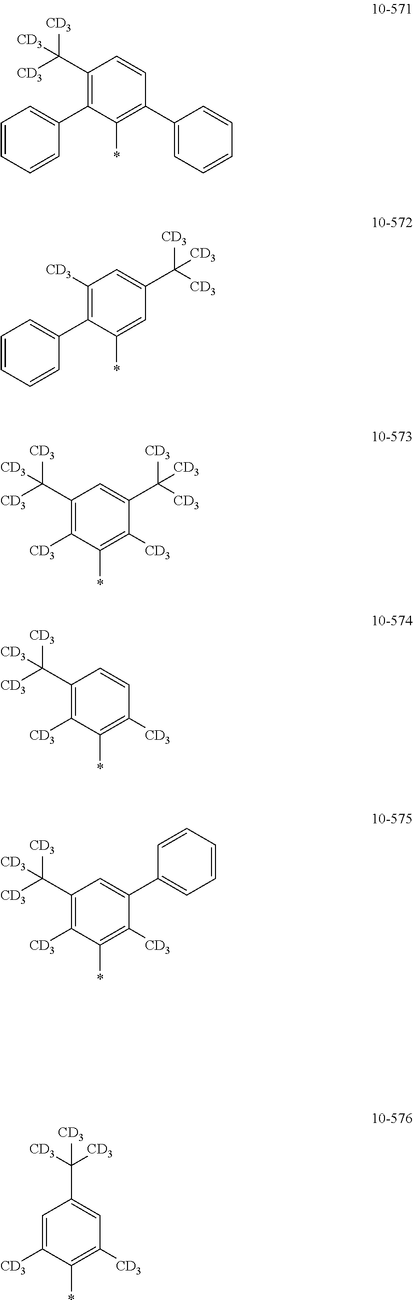

The “group represented by Formulae 10-1 to 10-130 in which at least one hydrogen is substituted with —F” and the “group represented by Formulae 10-201 to 10-358 in which at least one hydrogen is substituted with —F” may each be, for example, a group represented by one of Formulae 10-601 to 617:

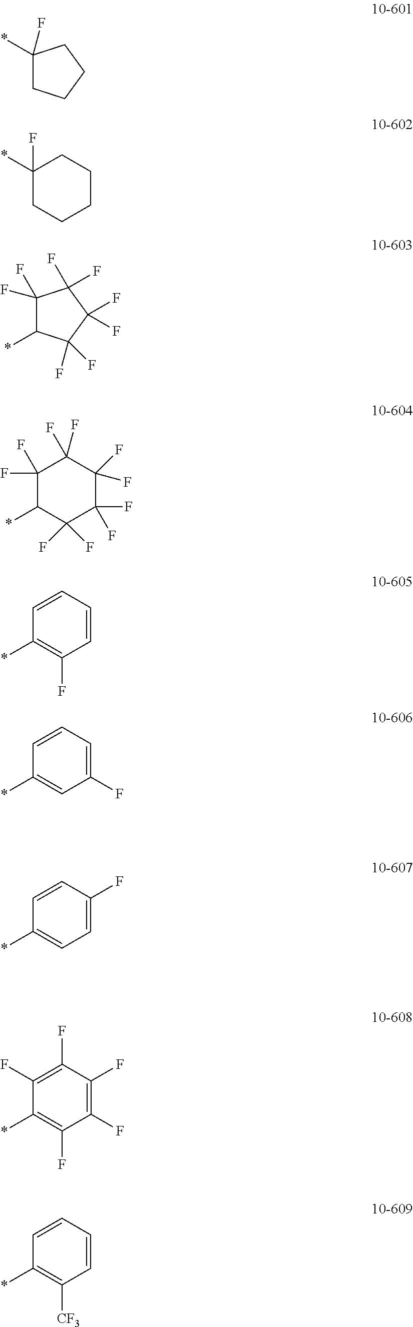

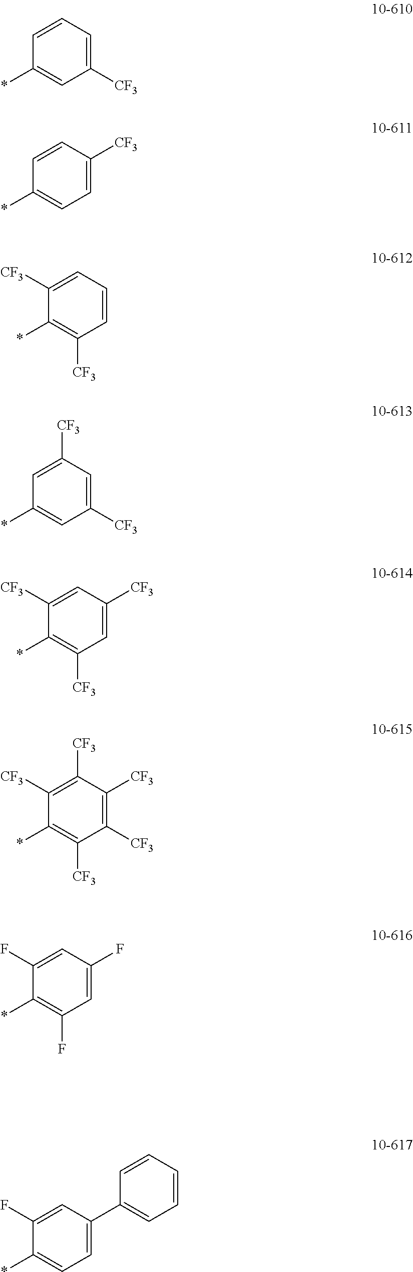

In Formulae 1 and 1A, R_{y} and R₁₁ to R₁₅ may each independently be a group represented by one of Formulae 2-1 to 2-3, hydrogen, deuterium, —F, —Cl, —Br, —I, —SFS, a hydroxyl group, a cyano group, a nitro group, an amidino group, a hydrazino group, a hydrazono group, a substituted or unsubstituted C₁-C₆₀ alkyl group, a substituted or unsubstituted C₂-C₆₀ alkenyl group, a substituted or unsubstituted C₂-C₆₀ alkynyl group, a substituted or unsubstituted C₁-C₆₀ alkoxy group, a substituted or unsubstituted C₃-C₁₀ cycloalkyl group, a substituted or unsubstituted C₁-C₁₀ heterocycloalkyl group, a substituted or unsubstituted C₃-C₁₀ cycloalkenyl group, a substituted or unsubstituted C₂-C₁₀ heterocycloalkenyl group, a substituted or unsubstituted C₆-C₆₀ aryl group, a substituted or unsubstituted C₇-C₆₀ alkylaryl group, a substituted or unsubstituted C₆-C₆₀ aryloxy group, a substituted or unsubstituted C₆-C₆₀ arylthio group, a substituted or unsubstituted C₁-C₆₀ heteroaryl group, a substituted or unsubstituted C₂-C₆₀ heteroalkylaryl group, a substituted or unsubstituted C₁-C₆₀ heteroaryloxy group, a substituted or unsubstituted C₁-C₆₀ heteroarylthio group, a substituted or unsubstituted monovalent non-aromatic condensed polycyclic group, a substituted or unsubstituted monovalent non-aromatic condensed heteropolycyclic group, —Si(Q₁)(Q₂)(Q₃), —C(Q₁)(Q₂)(Q₃), —B(Q₁)(Q₂), —N(Q₁)(Q₂), —P(Q₁)(Q₂), —C(═O)(Q₁), —S(═O)(Q₁), —S(═O)₂(Q₁), —P(═O)(Q₁)(Q₂), or —P(═S)(Q₁)(Q₂), and
Q₁ to Q₃ may each independently be hydrogen, deuterium, —F, —Cl, —Br, —I, a hydroxyl group, a cyano group, a nitro group, an amidino group, a hydrazino group, a hydrazono group, a C₁-C₆₀ alkyl group, a C₂-C₆₀ alkenyl group, a C₂-C₆₀ alkynyl group, a C₁-C₆₀ alkoxy group, a C₃-C₁₀ cycloalkyl group, a C₁-C₁₀ heterocycloalkyl group, a C₃-C₁₀ cycloalkenyl group, a C₂-C₁₀ heterocycloalkenyl group, a C₆-C₆₀ aryl group, a C₇-C₆₀ alkylaryl group, a C₆-C₆₀ aryloxy group, a C₆-C₆₀ arylthio group, a C₁-C₆₀ heteroaryl group, a C₂-C₆₀ heteroalkylaryl group, a C₁-C₆₀ heteroaryloxy group, a C₁-C₆₀ heteroarylthio group, a monovalent non-aromatic condensed polycyclic group, a monovalent non-aromatic condensed heteropolycyclic group, a C₁-C₆₀ alkyl group substituted with at least one of deuterium, —F, a cyano group, a C₁-C₆₀ alkyl group, a C₆-C₆₀ aryl group, or a combination thereof, or a C₆-C₆₀ aryl group substituted with at least one of deuterium, —F, a cyano group, a C₁-C₆₀ alkyl group, a C₆-C₆₀ aryl group, or a combination thereof.

In an embodiment, in Formulae 1 and 1A, R_{y} and R₁₁ to R₁₅ may each independently be groups represented by Formulae 2-1 to 2-3 and/or R₂₃ may be defined the same as described above.

In an embodiment, Rₓ and R_{y} may each independently be a group represented by one of Formulae 2-1 to 2-3.

In Formulae 1 and 1A, b11 to b14 may each independently be 0, 1, 2, 3, or 4. In Formulae 1 and 1A, b11 to b14 indicate the number of occurrences of R₁₁ to R₁₄, respectively.

In Formulae 1 and 1A, b15 may be 0, 1, or 2. In Formulae 1 and 1A, b15 indicates the number of occurrences of R₁₅.

In an embodiment, the heterocyclic compound may be represented by one of Formulae 11 and 12:

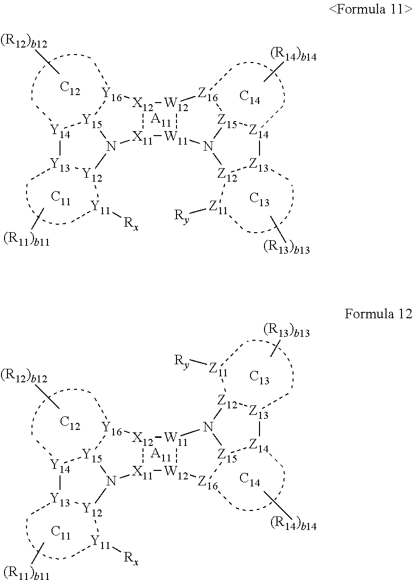

wherein, in Formulae 11 and 12,
X₁₁, X₁₂, Y₁₁ to Y₁₆, Z₁₁ to Z₁₆, W₁₁, W₁₂, A₁₁, C₁₁ to C₁₄, Rₓ, R_{y}, R₁₁ to R₁₄, and b₁₁ to b₁₄ may each be the same as described in Formulae 1 and 1A.

For example, in Formulae 11 and 12, A₁₁ may be groups represented by Formulae 3-11 to 3-16.

In one or more embodiments, the heterocyclic compound may be represented by Formulae 11-1 or 12-1:

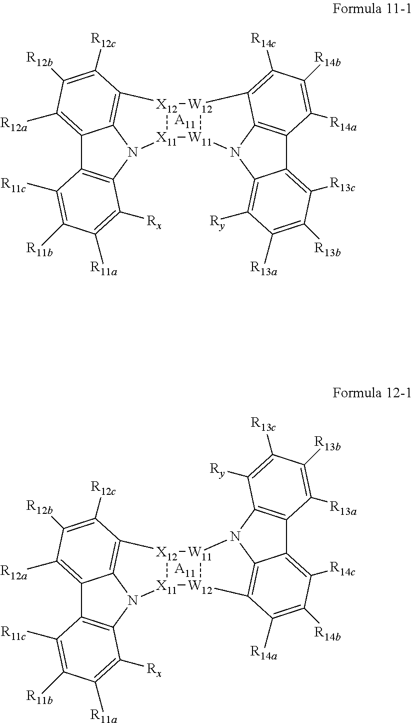

wherein, in Formulae 11-1 and 12-1,
X₁₁, X₁₂, W₁₁, W₁₂, A₁₁, Rₓ, and R_{y} may each be the same as described in Formulae 1 and 1A,
R₁₁ₐ to R_{11c}, R₁₂ₐ to R_{12c}, R₁₃ₐ to R_{13c}, and R₁₄ₐ to R_{14c} may each be the same as described in connection with R₁₁ in Formula 1, and any neighboring two of R₁₁ₐ to R_{11c}, R₁₂ₐ to R_{12c}, R₁₃ₐ to R_{13c}, and R₁₄ₐ to R_{14c} may optionally be combined with each other to form a ring.

In one or more embodiments, the heterocyclic compound may be a compound of Group C:

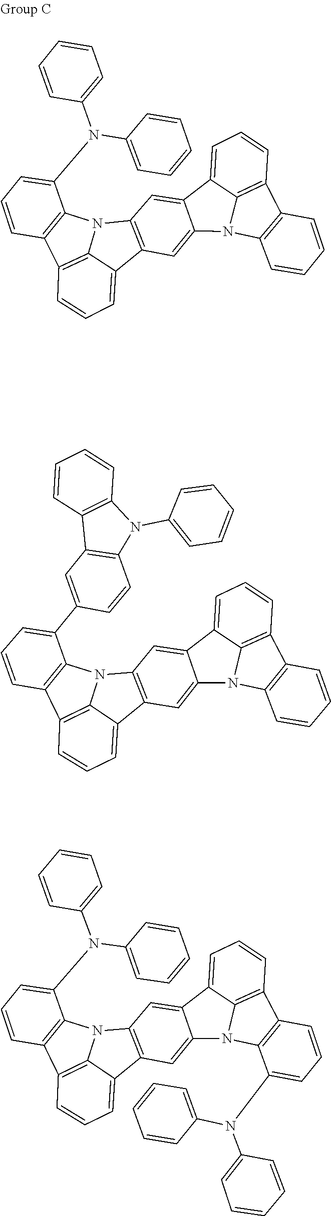 [SMILES: c1ccc(N(c2ccccc2)c2cccc3c4cccc5c6cc7c(cc6n(c23)c54)c2cccc3c4ccccc4n7c32)cc1 ; c1ccc(-n2c3ccccc3c3cc(-c4cccc5c6cccc7c8cc9c(cc8n(c45)c76)c4cccc5c6ccccc6n9c54)ccc32)cc1 ; c1ccc(N(c2ccccc2)c2cccc3c4cccc5c6cc7c(cc6n(c23)c54)c2cccc3c4cccc(N(c5ccccc5)c5ccccc5)c4n7c23)cc1]

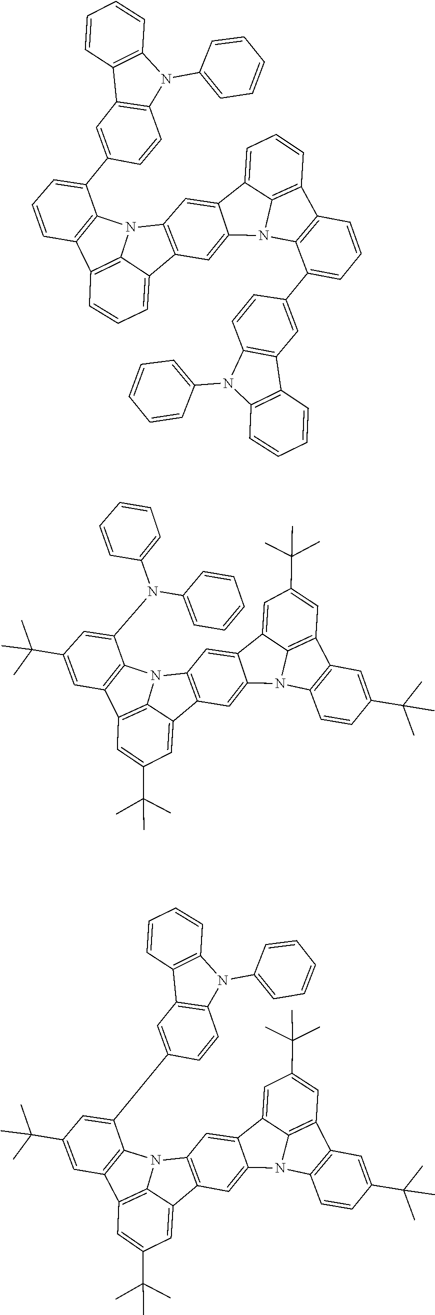

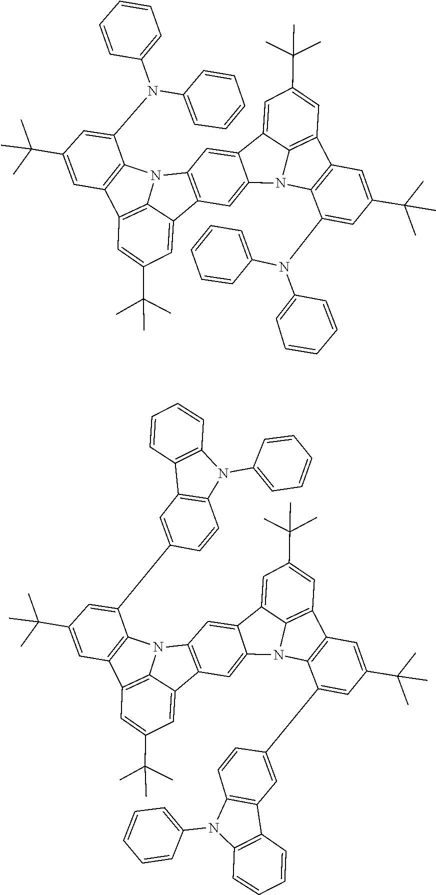

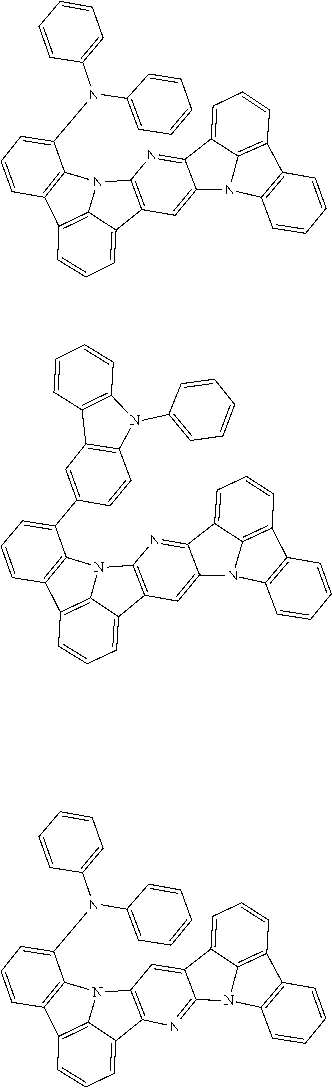 [SMILES: c1ccc(N(c2ccccc2)c2cccc3c4cccc5c6cc7c(nc6n(c23)c54)c2cccc3c4ccccc4n7c32)cc1 ; c1ccc(-n2c3ccccc3c3cc(-c4cccc5c6cccc7c8cc9c(nc8n(c45)c76)c4cccc5c6ccccc6n9c54)ccc32)cc1 ; c1ccc(N(c2ccccc2)c2cccc3c4cccc5c6nc7c(cc6n(c23)c45)c2cccc3c4ccccc4n7c32)cc1]

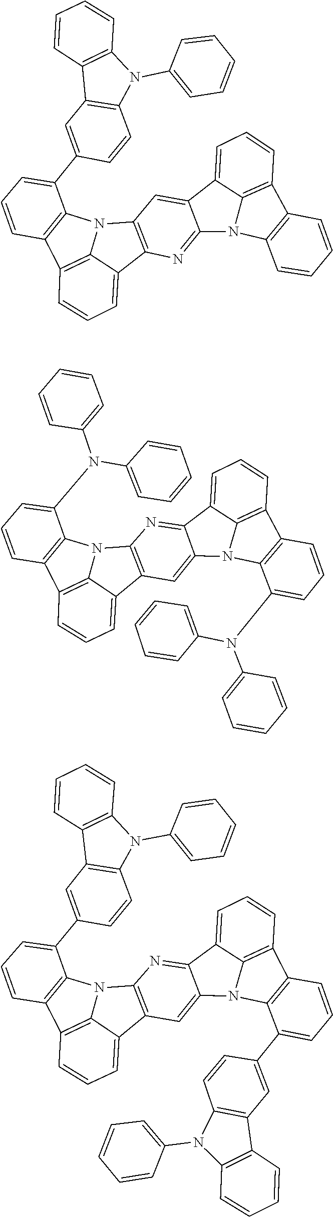 [SMILES: c1ccc(-n2c3ccccc3c3cc(-c4cccc5c6cccc7c8nc9c(cc8n(c45)c67)c4cccc5c6ccccc6n9c54)ccc32)cc1 ; c1ccc(N(c2ccccc2)c2cccc3c4cccc5c6nc7c(cc6n(c23)c45)c2cccc3c4cccc(N(c5ccccc5)c5ccccc5)c4n7c23)cc1 ; c1ccc(-n2c3ccccc3c3cc(-c4cccc5c6cccc7c8nc9c(cc8n(c45)c67)c4cccc5c6cccc(-c7ccc8c(c7)c7ccccc7n8-c7ccccc7)c6n9c45)ccc32)cc1]

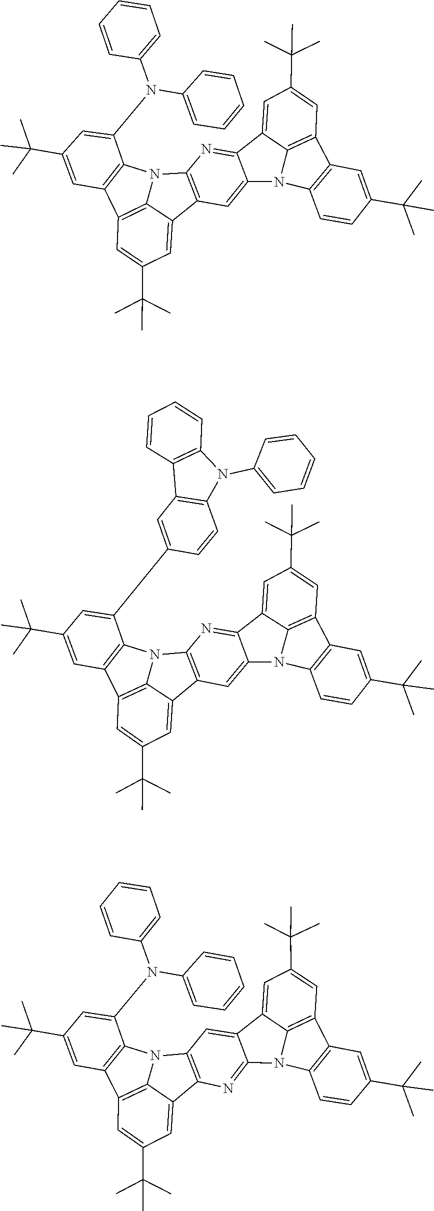

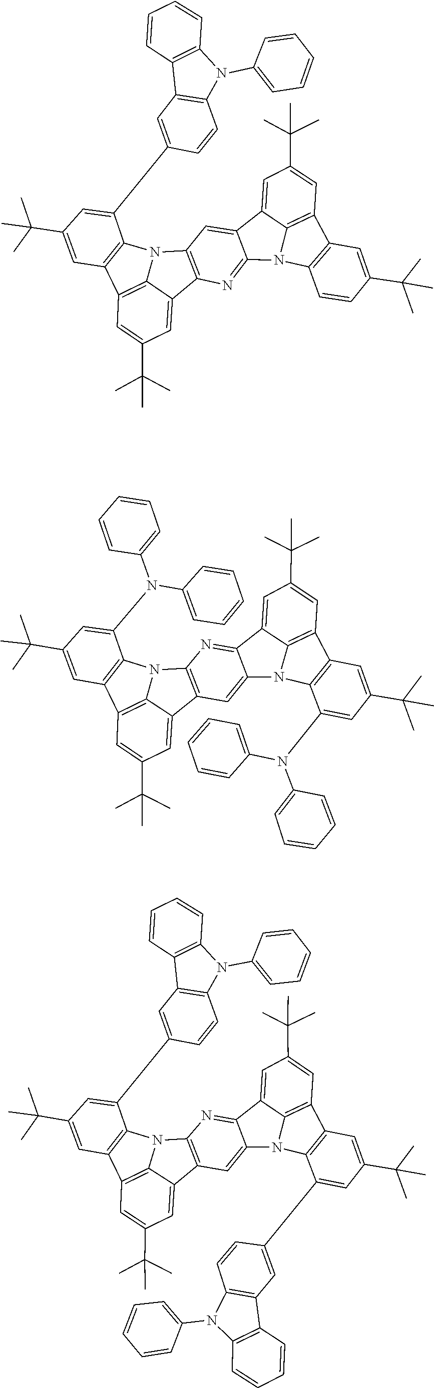

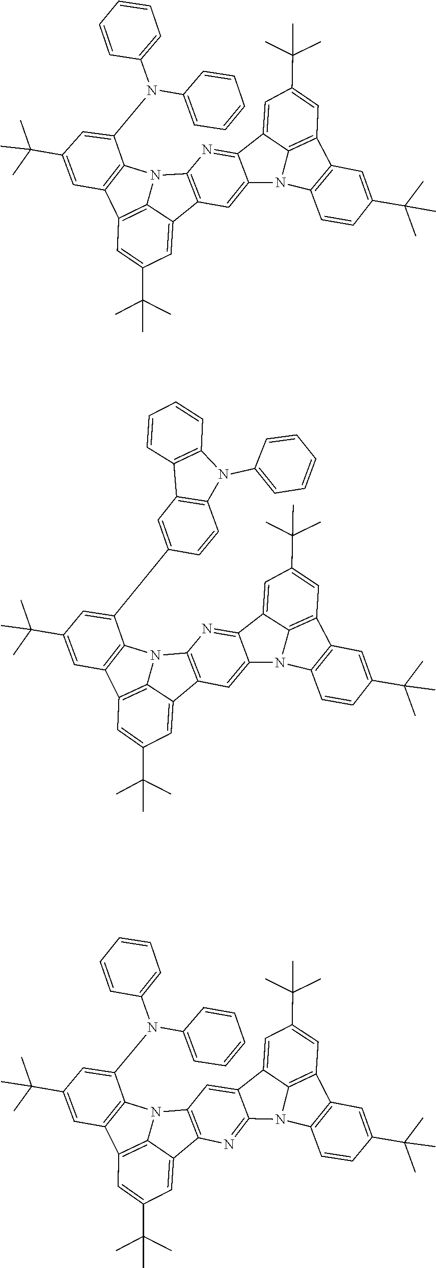

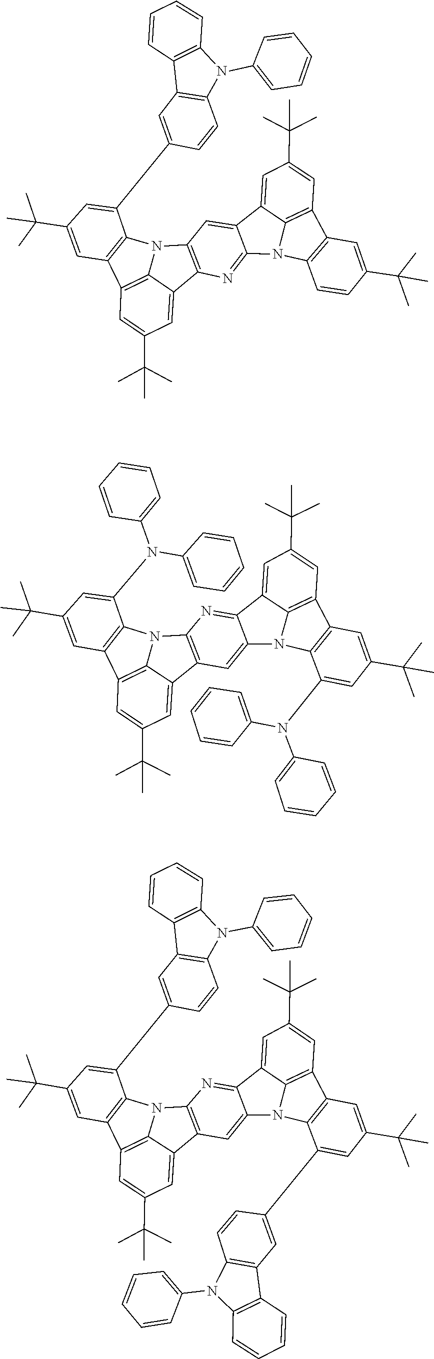

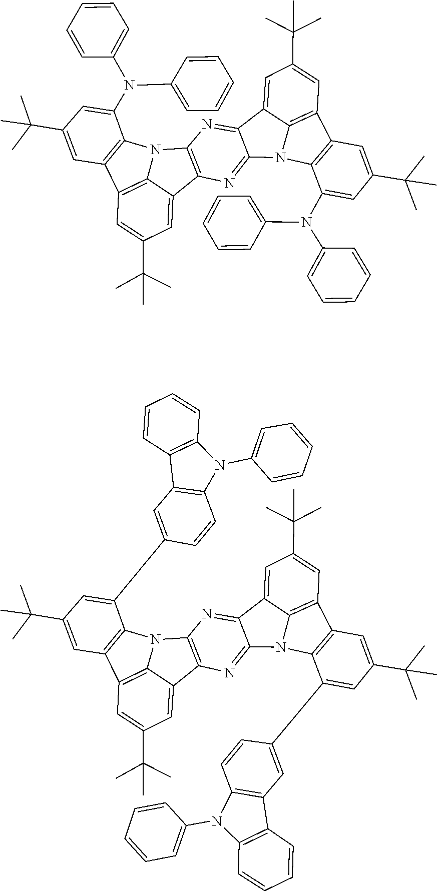

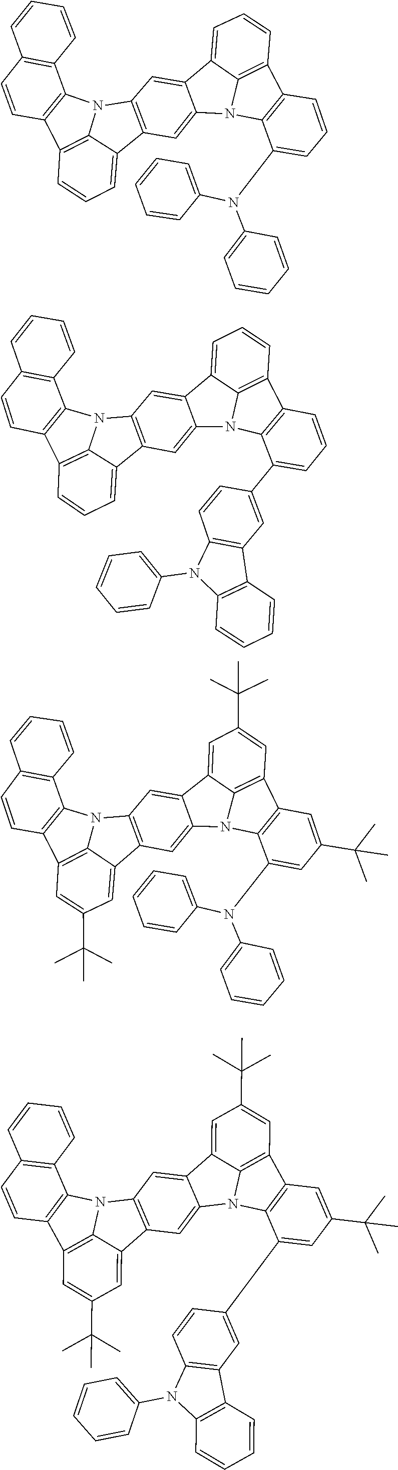

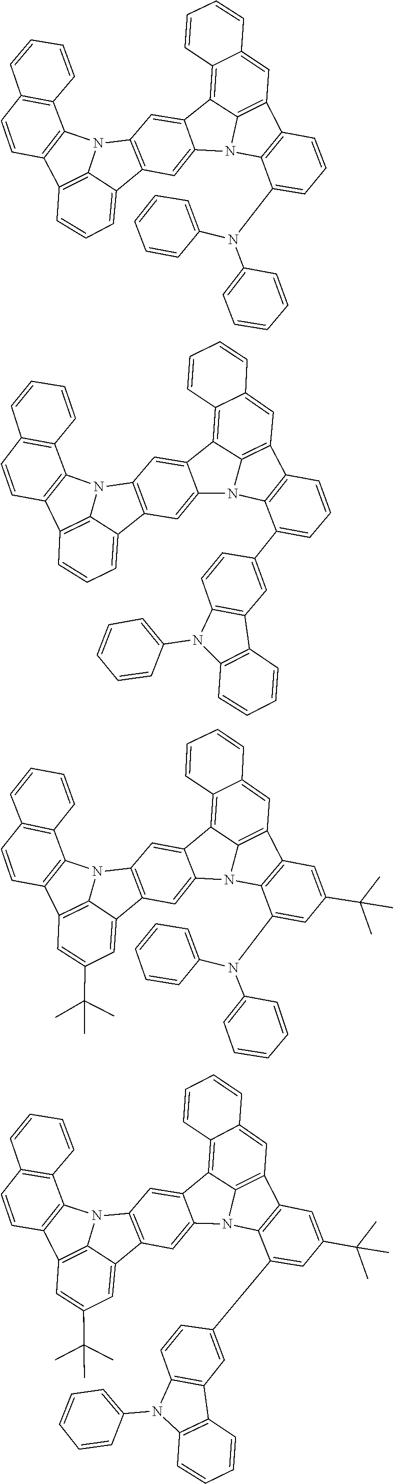

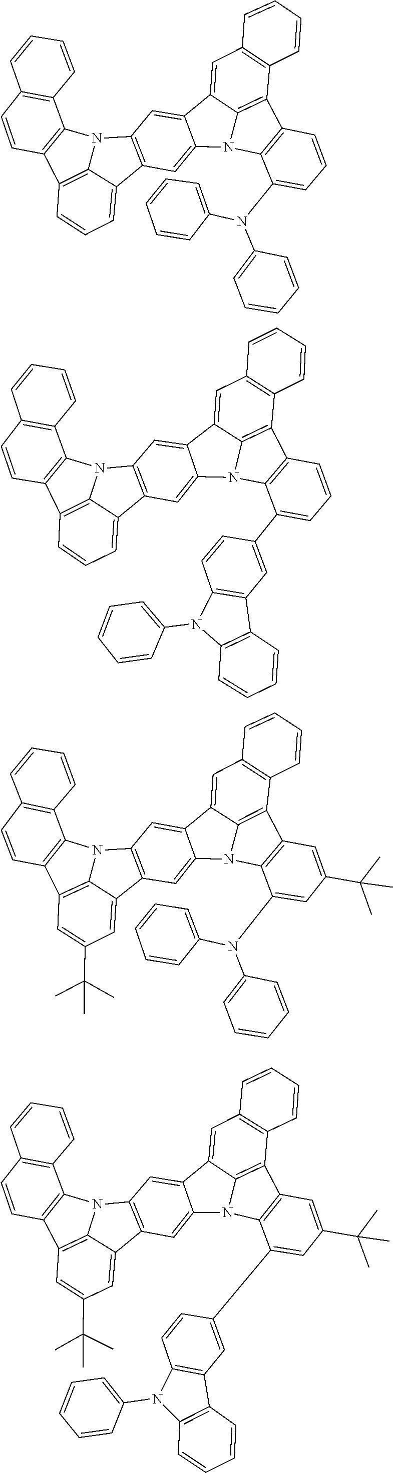

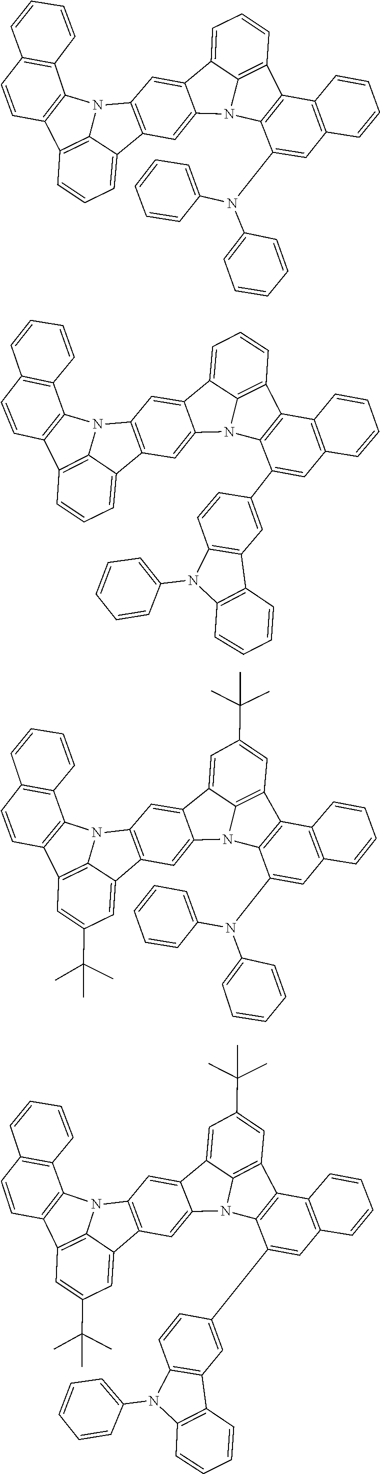

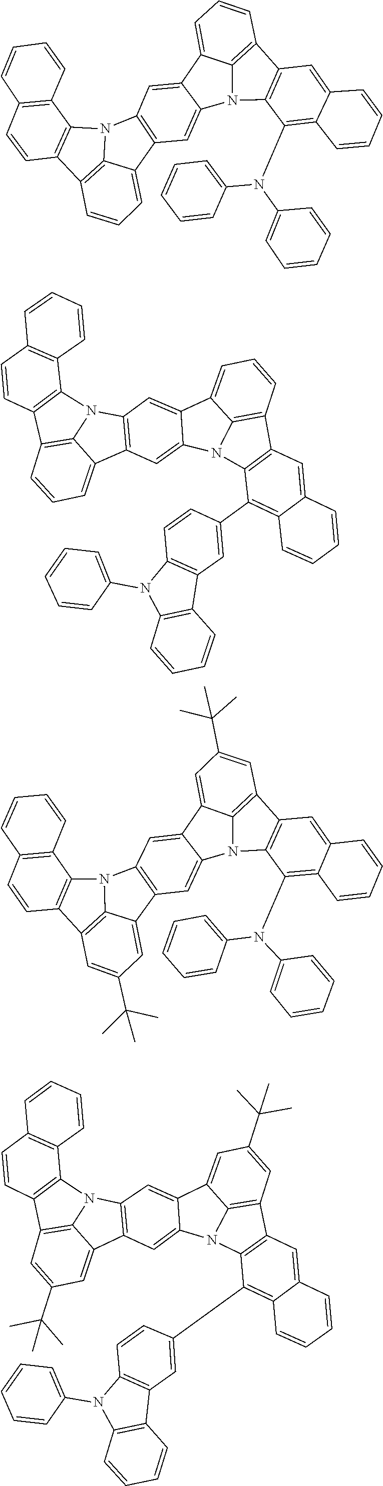

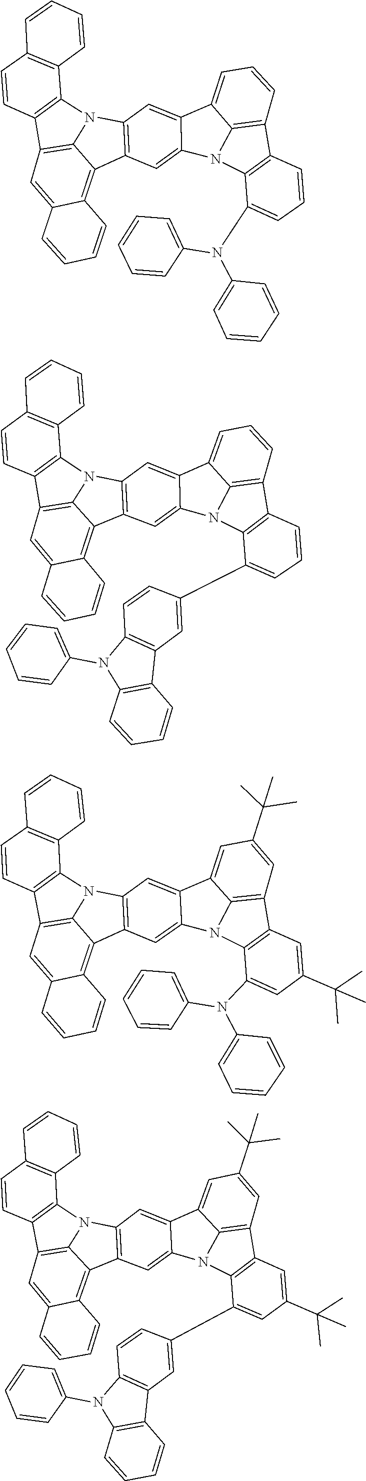

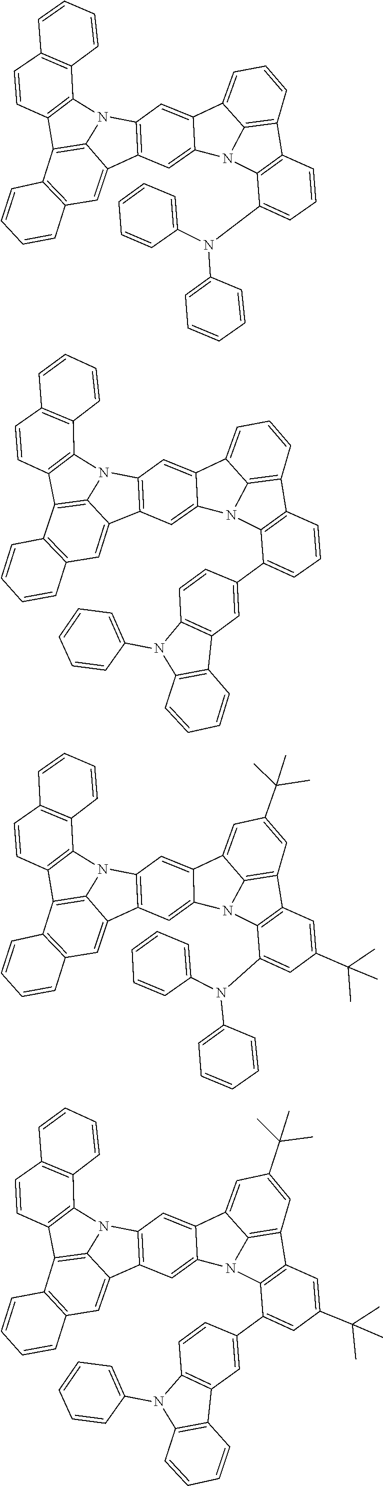

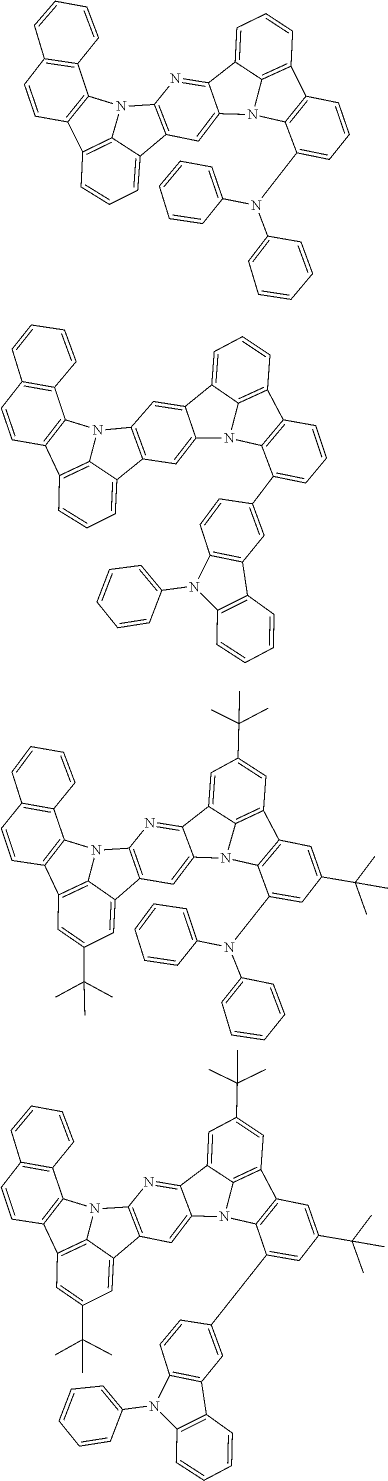

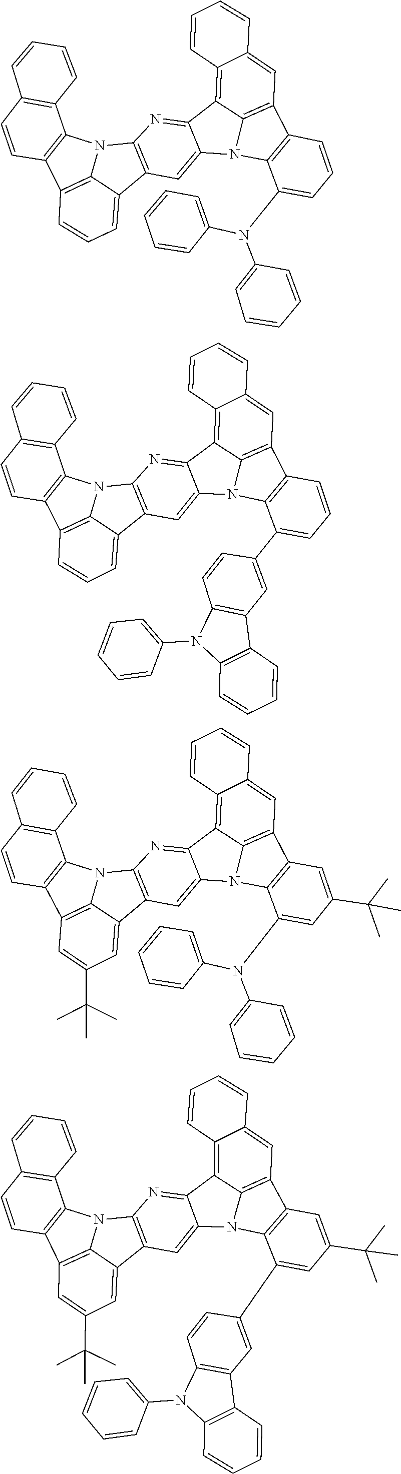

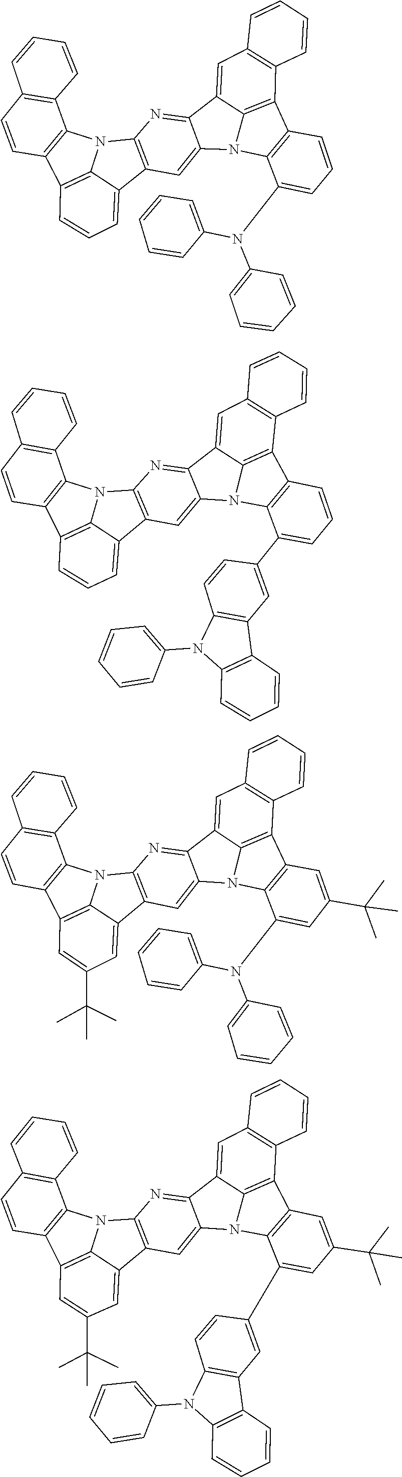

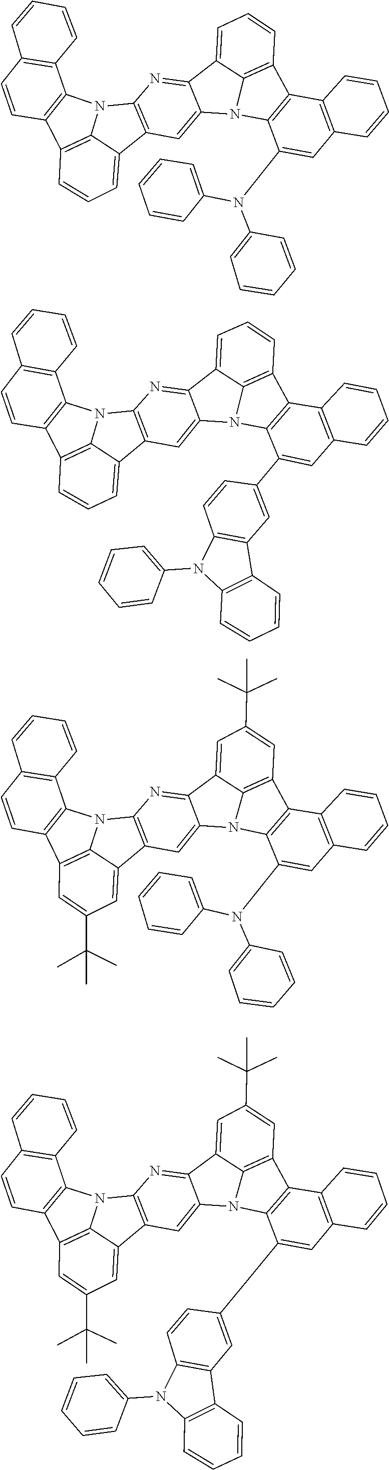

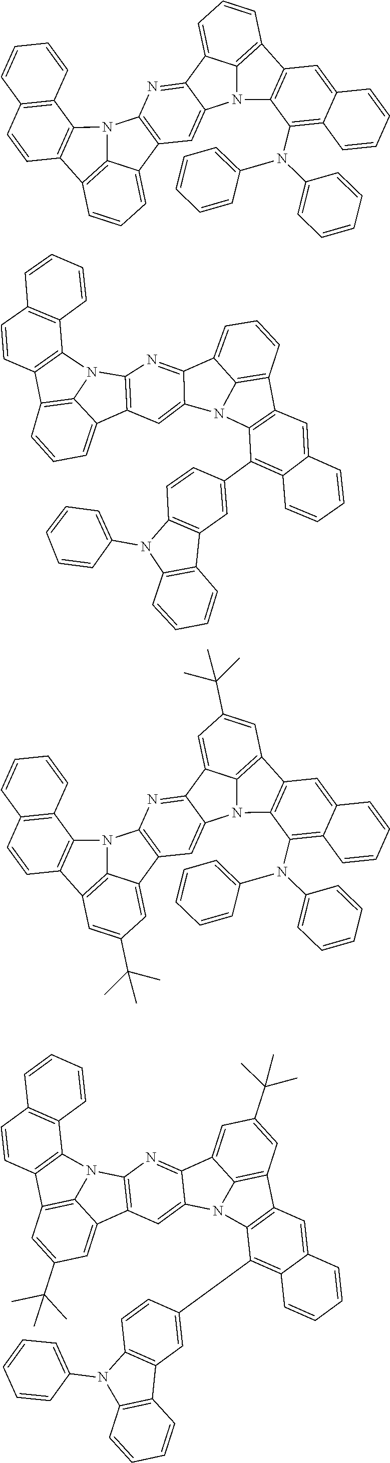

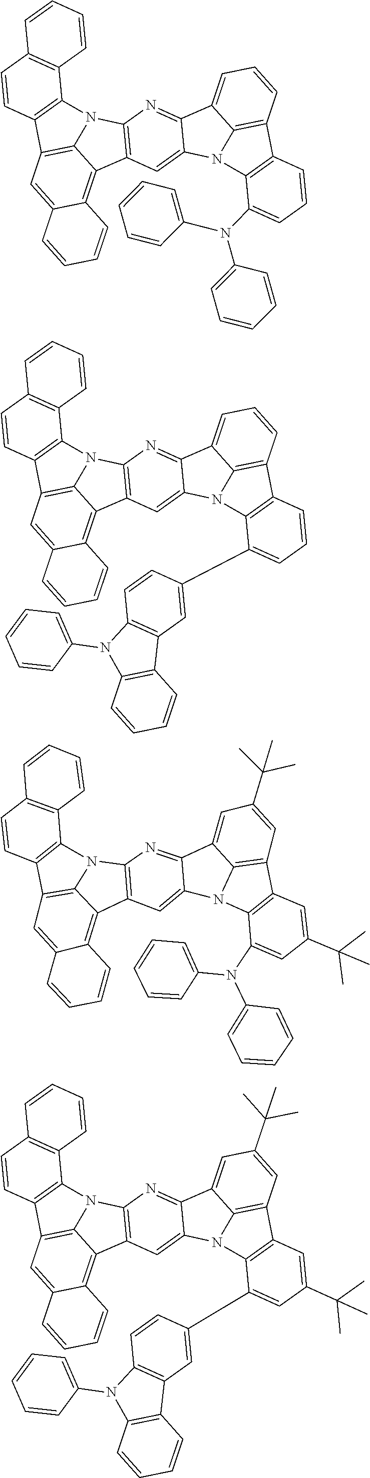

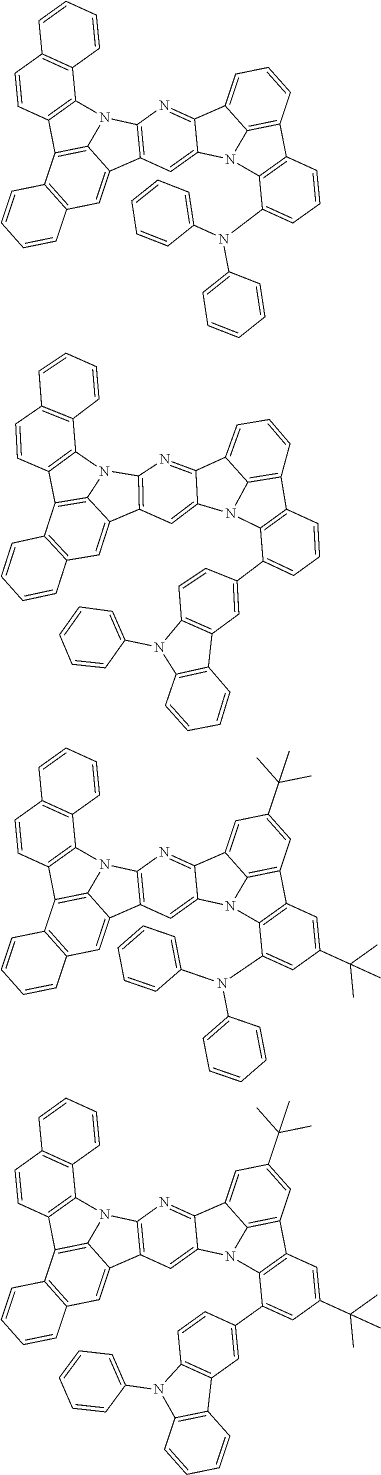

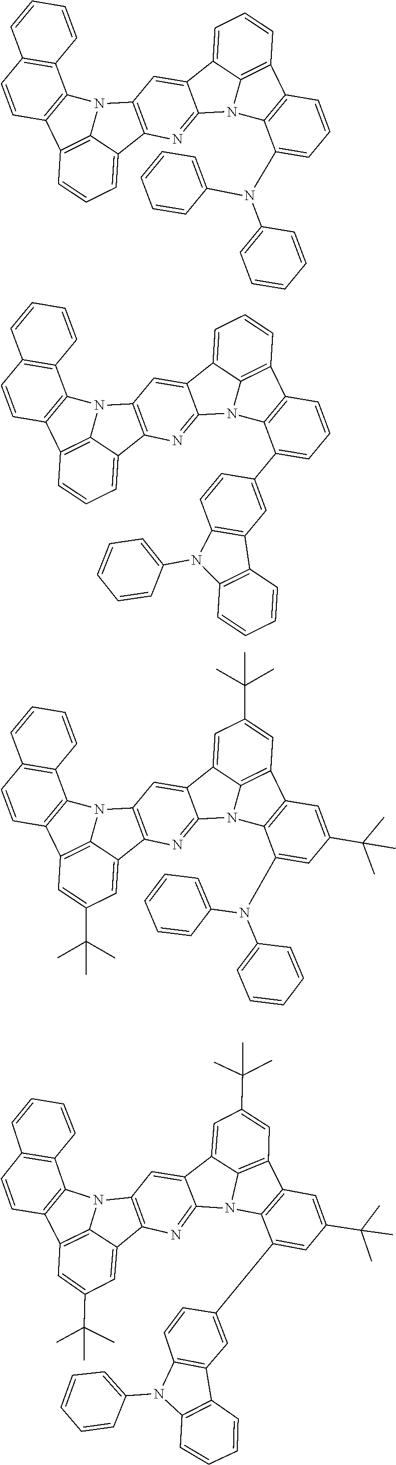

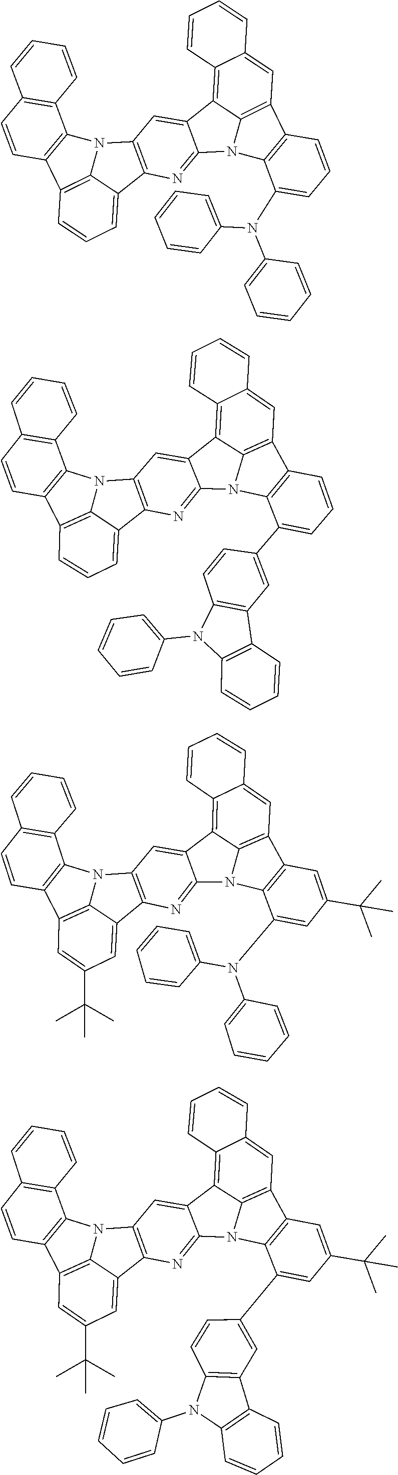

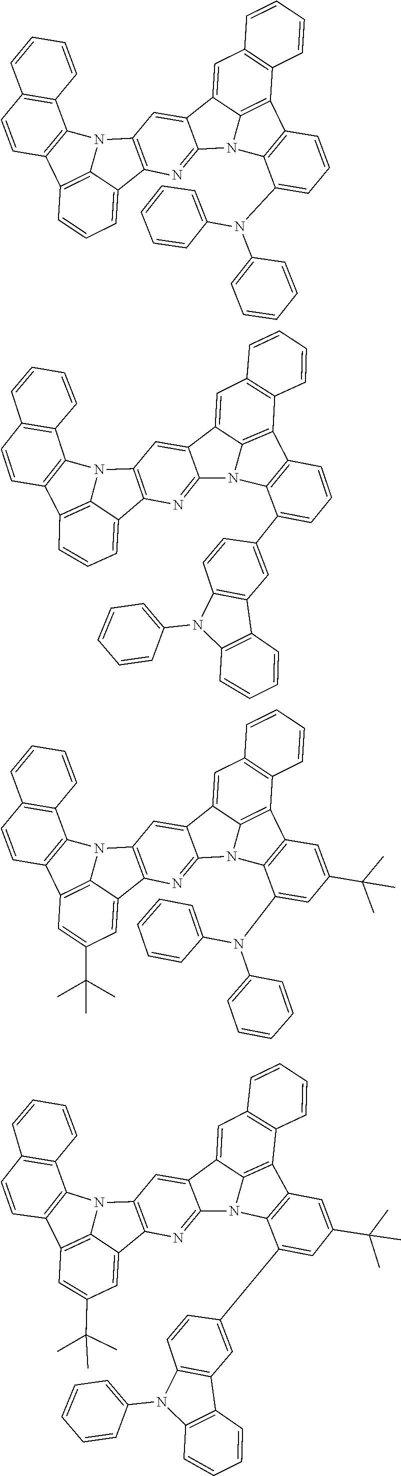

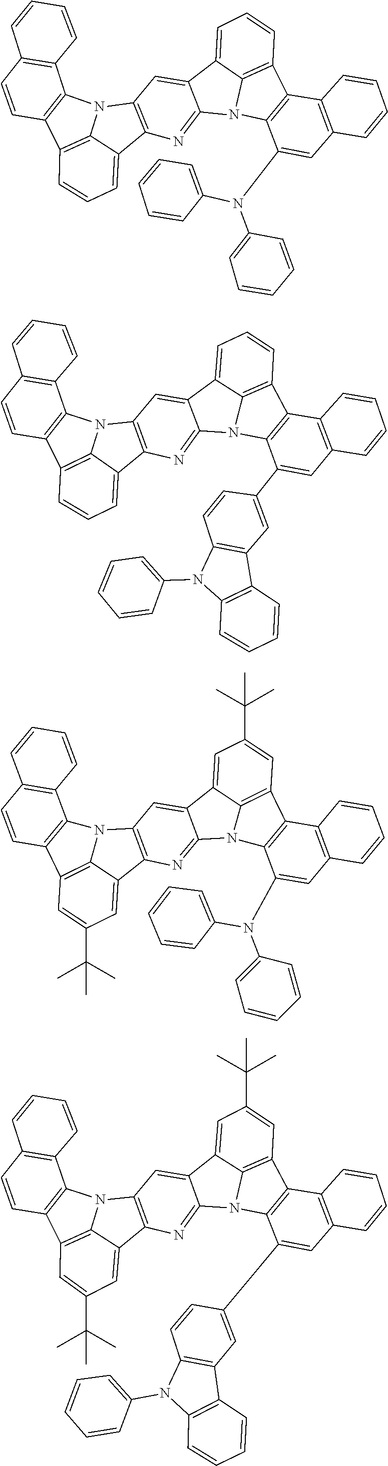

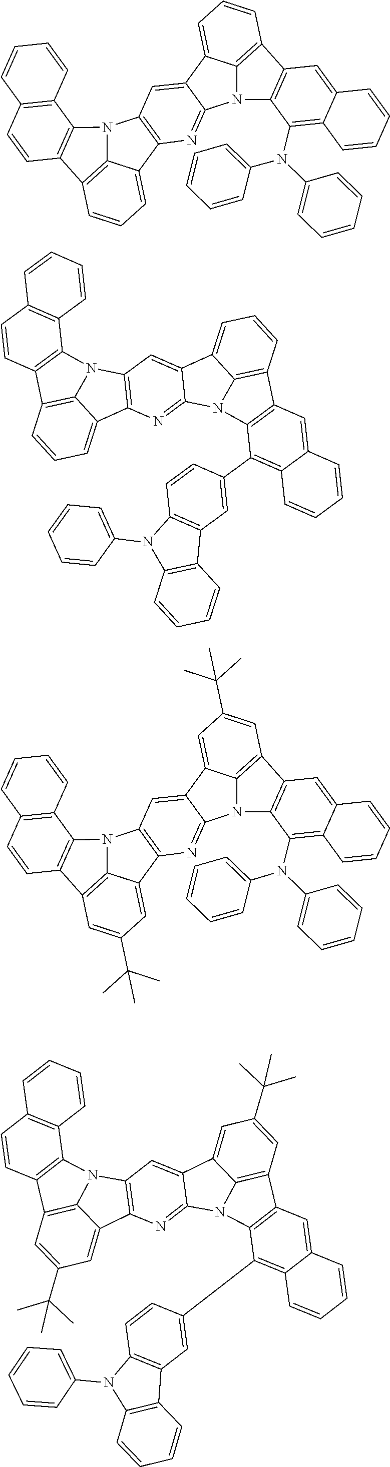

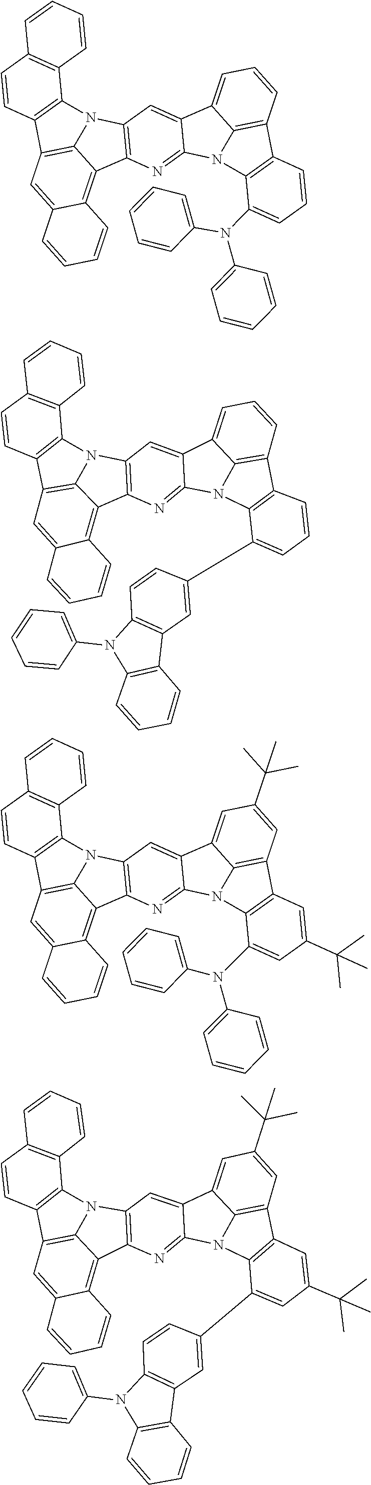

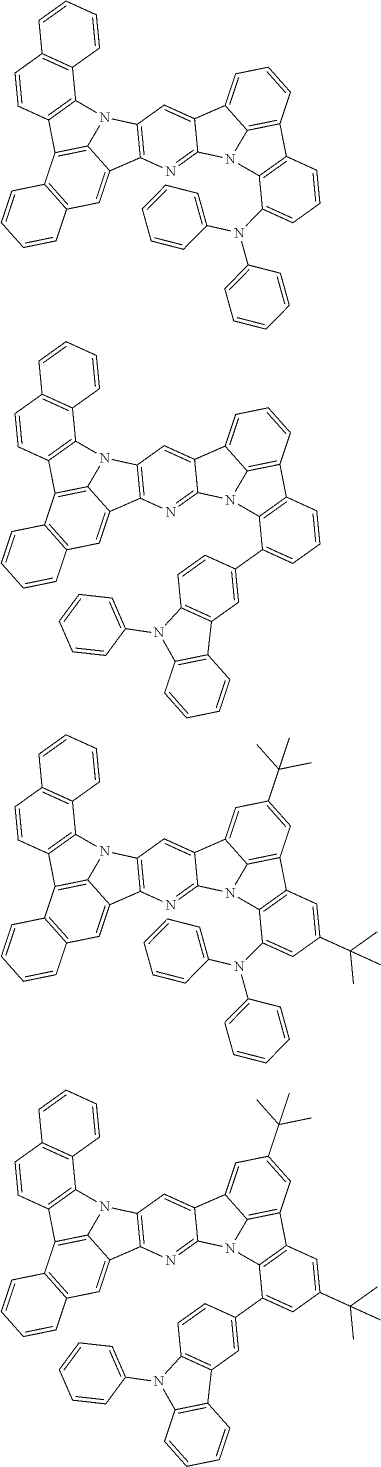

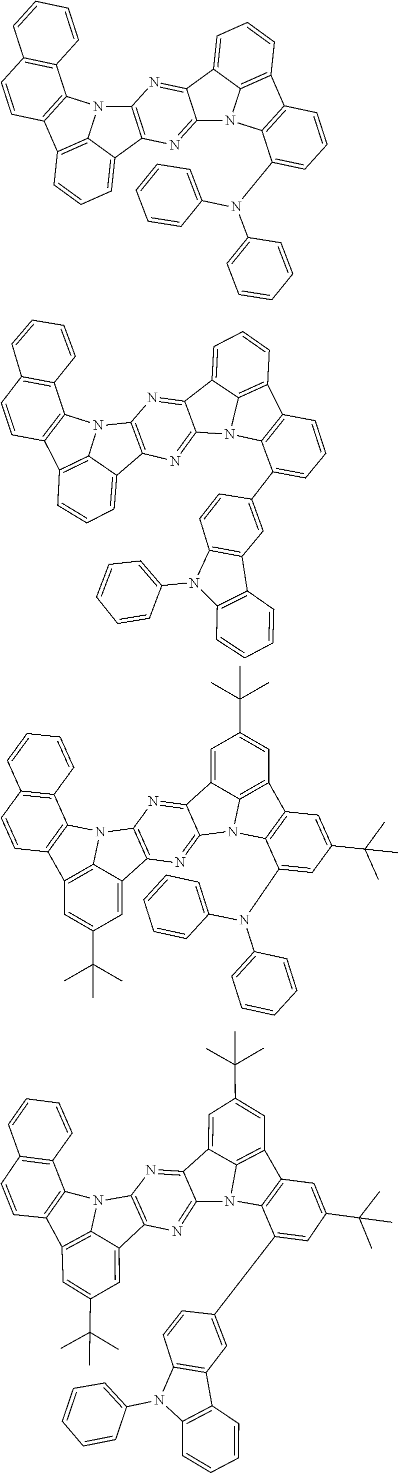

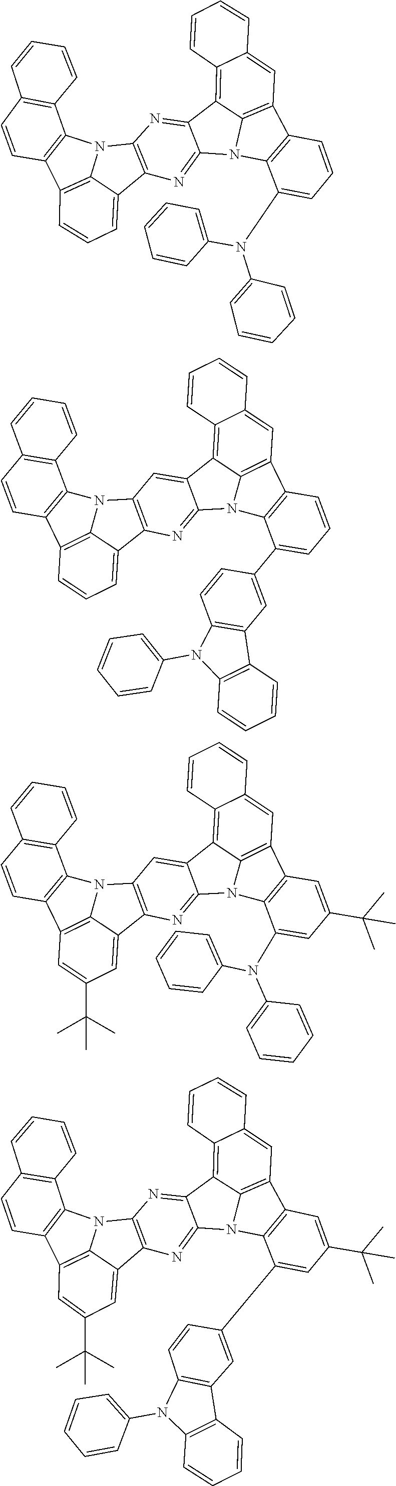

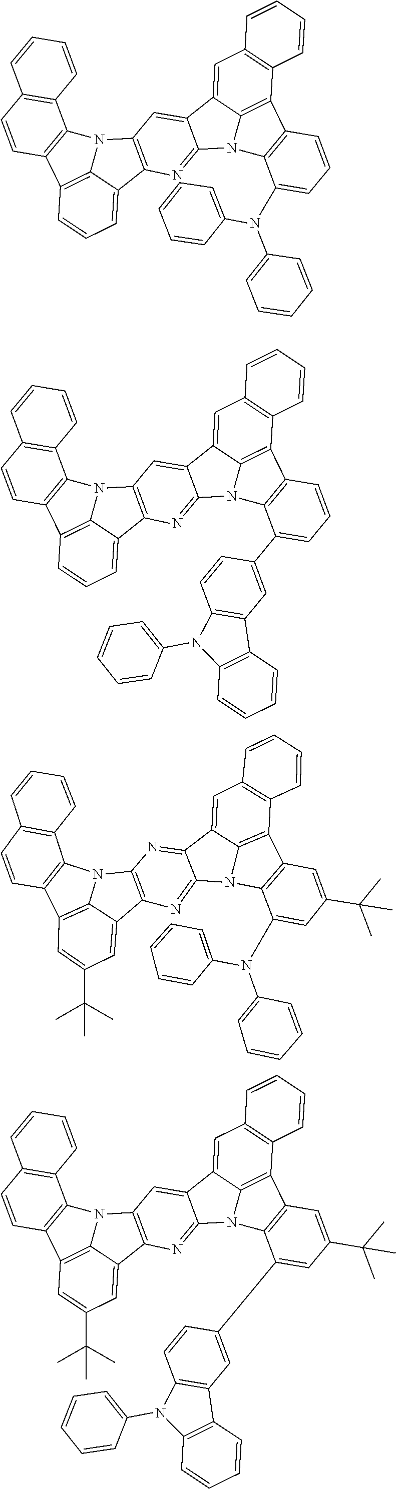

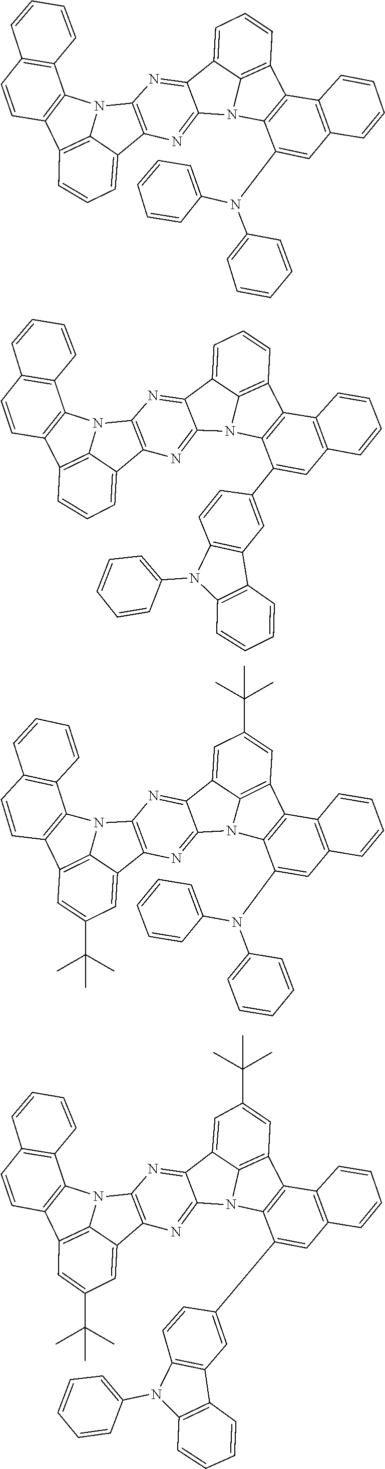

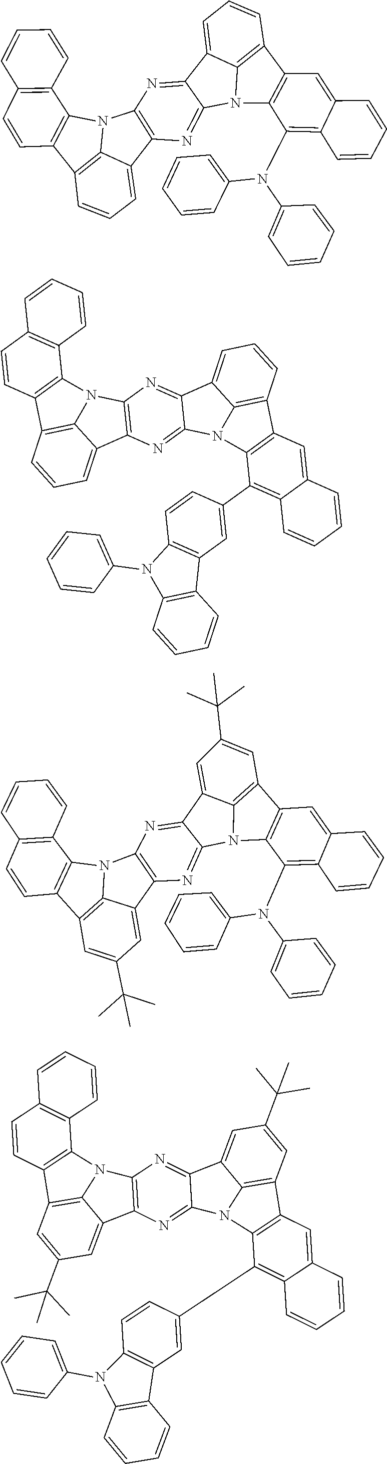

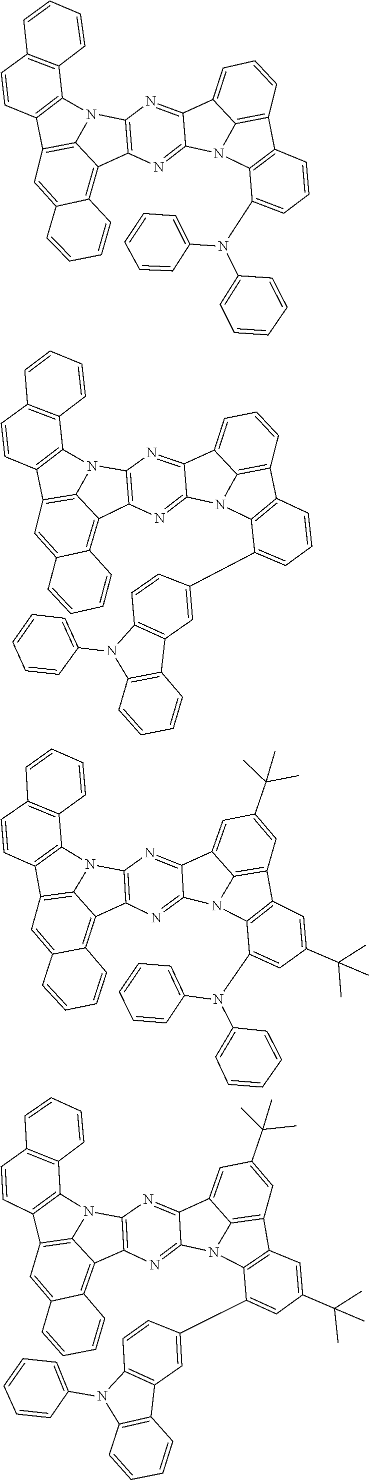

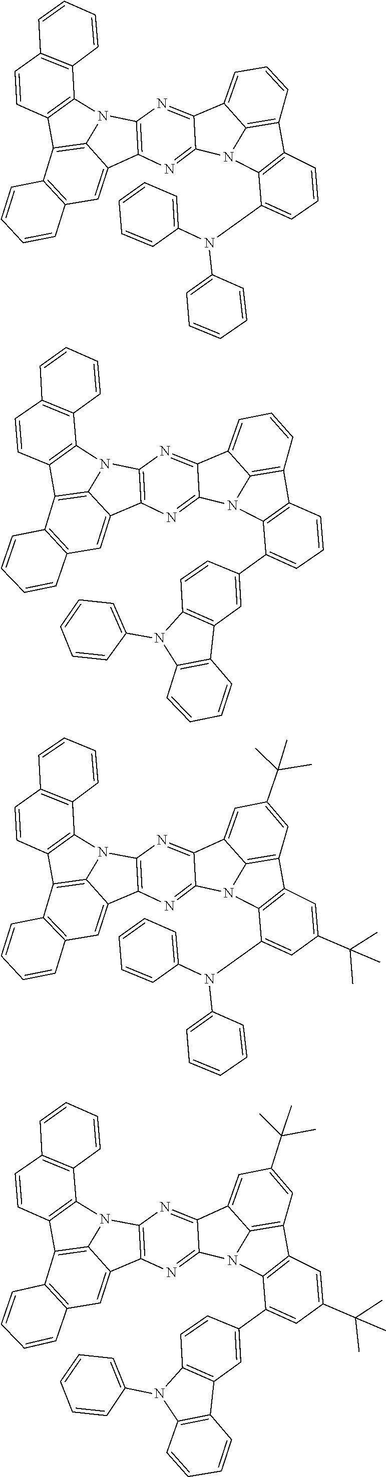 [SMILES: c1ccc(N(c2ccccc2)c2cccc3c4cccc5c6nc7c(nc6n(c23)c45)c2cc3ccccc3c3c4ccc5ccccc5c4n7c23)cc1 ; c1ccc(-n2c3ccccc3c3cc(-c4cccc5c6cccc7c8nc9c(nc8n(c45)c67)c4cc5ccccc5c5c6ccc7ccccc7c6n9c45)ccc32)cc1 ; CC(C)(C)c1cc(N(c2ccccc2)c2ccccc2)c2c(c1)c1cc(C(C)(C)C)cc3c4nc5c(nc4n2c13)c1cc2ccccc2c2c3ccc4ccccc4c3n5c12 ; CC(C)(C)c1cc(-c2ccc3c(c2)c2ccccc2n3-c2ccccc2)c2c(c1)c1cc(C(C)(C)C)cc3c4nc5c(nc4n2c13)c1cc2ccccc2c2c3ccc4ccccc4c3n5c12]

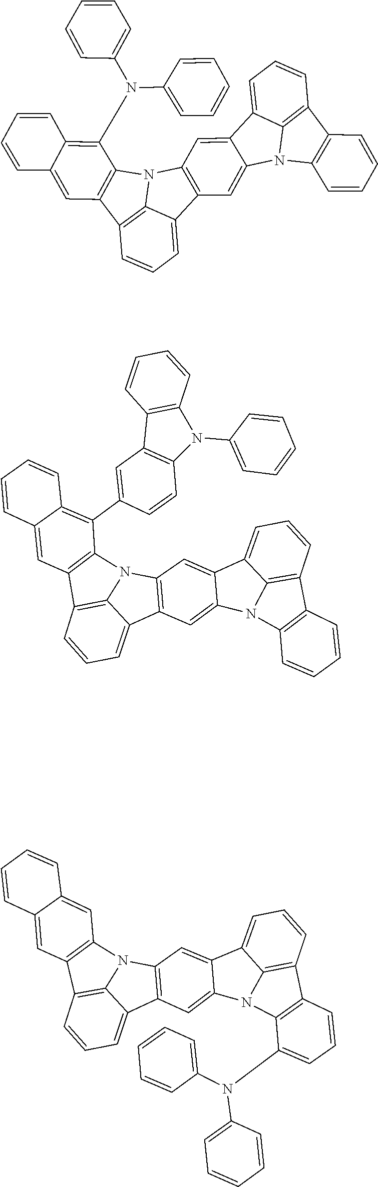 [SMILES: c1ccc(N(c2ccccc2)c2c3ccccc3cc3c4cccc5c6cc7c(cc6n(c23)c54)c2cccc3c4ccccc4n7c32)cc1 ; c1ccc(-n2c3ccccc3c3cc(-c4c5ccccc5cc5c6cccc7c8cc9c(cc8n(c45)c76)c4cccc5c6ccccc6n9c54)ccc32)cc1 ; c1ccc(N(c2ccccc2)c2cccc3c4cccc5c6cc7c(cc6n(c23)c54)c2cccc3c4cc5ccccc5cc4n7c32)cc1]

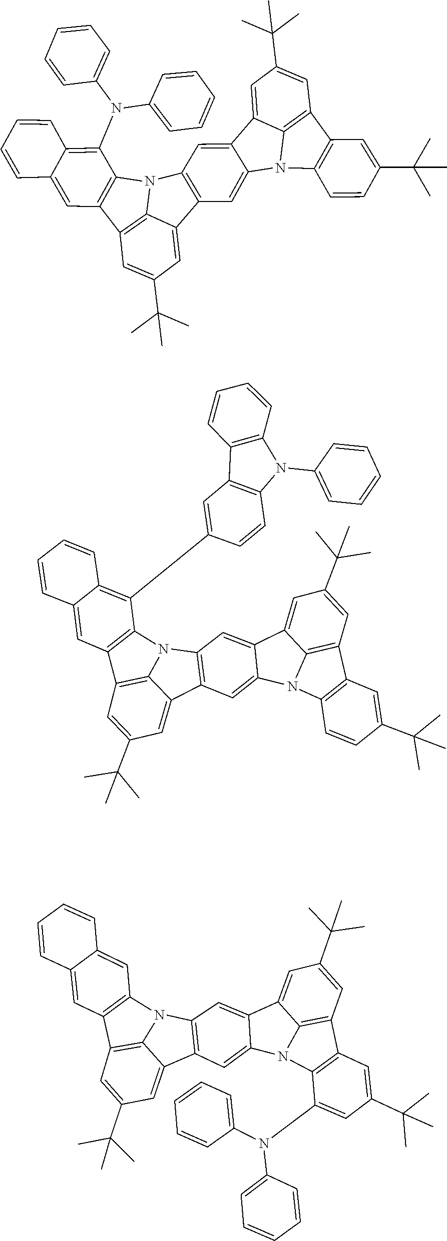 [SMILES: CC(C)(C)c1ccc2c(c1)c1cc(C(C)(C)C)cc3c4cc5c(cc4n2c13)c1cc(C(C)(C)C)cc2c3cc4ccccc4c(N(c4ccccc4)c4ccccc4)c3n5c12 ; CC(C)(C)c1ccc2c(c1)c1cc(C(C)(C)C)cc3c4cc5c(cc4n2c13)c1cc(C(C)(C)C)cc2c3cc4ccccc4c(-c4ccc6c(c4)c4ccccc4n6-c4ccccc4)c3n5c12 ; CC(C)(C)c1cc(N(c2ccccc2)c2ccccc2)c2c(c1)c1cc(C(C)(C)C)cc3c4cc5c(cc4n2c31)c1cc(C(C)(C)C)cc2c3cc4ccccc4cc3n5c21]

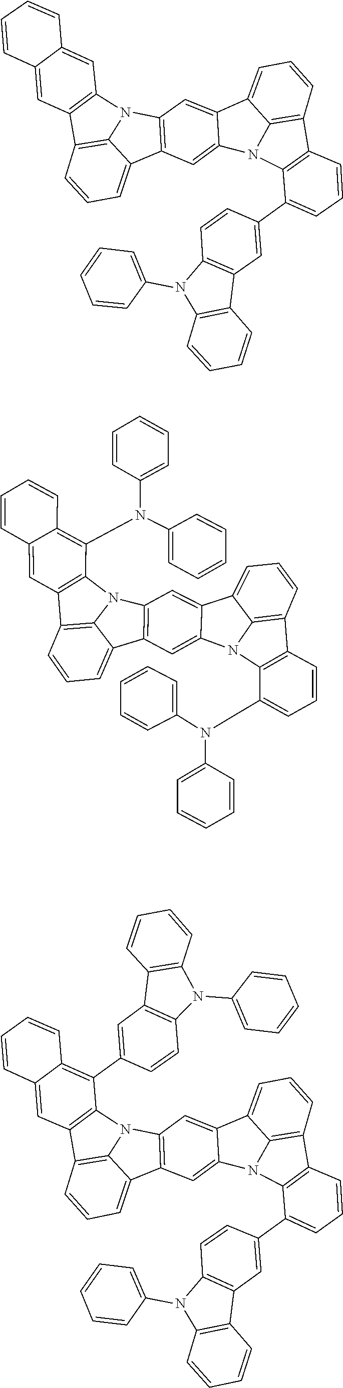 [SMILES: c1ccc(-n2c3ccccc3c3cc(-c4cccc5c6cccc7c8cc9c(cc8n(c45)c76)c4cccc5c6cc7ccccc7cc6n9c54)ccc32)cc1 ; c1ccc(N(c2ccccc2)c2cccc3c4cccc5c6cc7c(cc6n(c23)c54)c2cccc3c4cc5ccccc5c(N(c5ccccc5)c5ccccc5)c4n7c23)cc1 ; c1ccc(-n2c3ccccc3c3cc(-c4cccc5c6cccc7c8cc9c(cc8n(c45)c76)c4cccc5c6cc7ccccc7c(-c7ccc8c(c7)c7ccccc7n8-c7ccccc7)c6n9c45)ccc32)cc1]

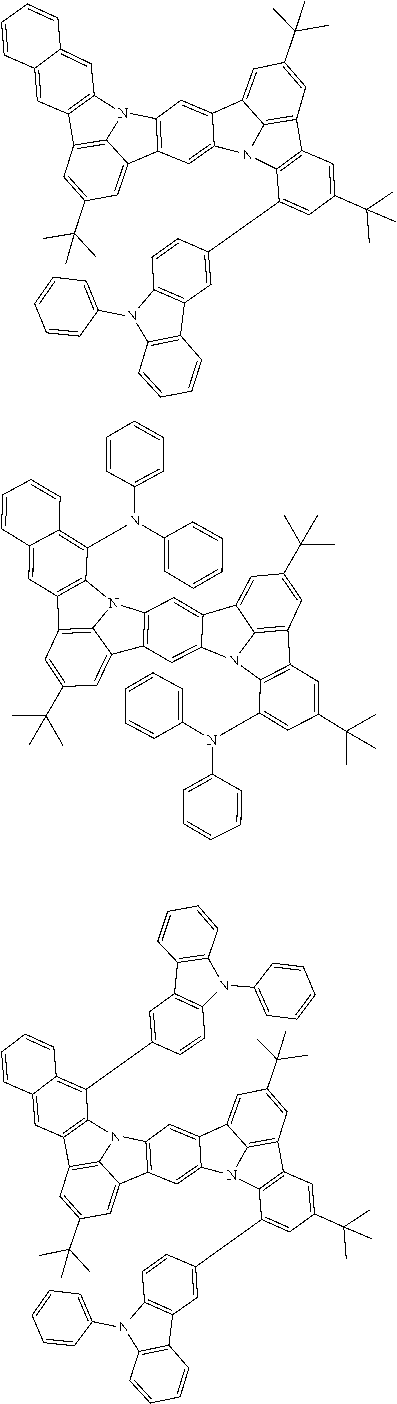

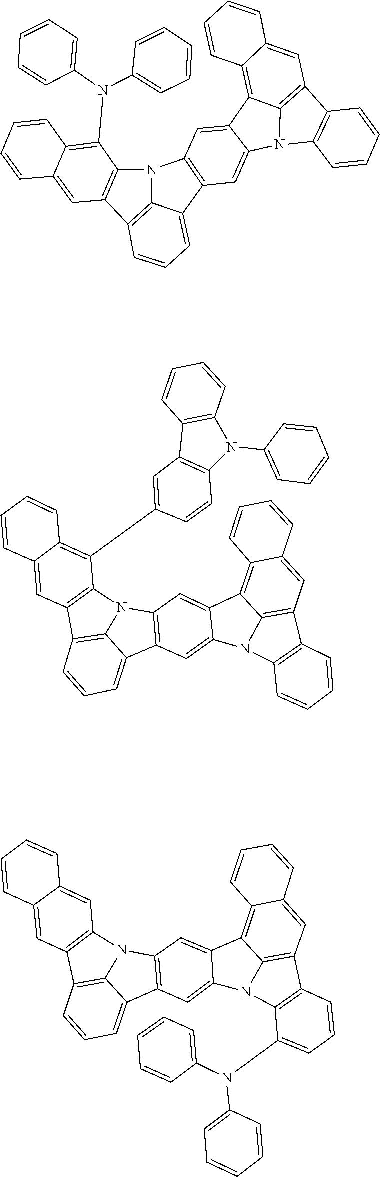 [SMILES: c1ccc(N(c2ccccc2)c2c3ccccc3cc3c4cccc5c6cc7c(cc6n(c23)c54)c2c3ccccc3cc3c4ccccc4n7c32)cc1 ; c1ccc(-n2c3ccccc3c3cc(-c4c5ccccc5cc5c6cccc7c8cc9c(cc8n(c45)c76)c4c5ccccc5cc5c6ccccc6n9c54)ccc32)cc1 ; c1ccc(N(c2ccccc2)c2cccc3c4cc5ccccc5c5c6cc7c(cc6n(c23)c45)c2cccc3c4cc5ccccc5cc4n7c32)cc1]

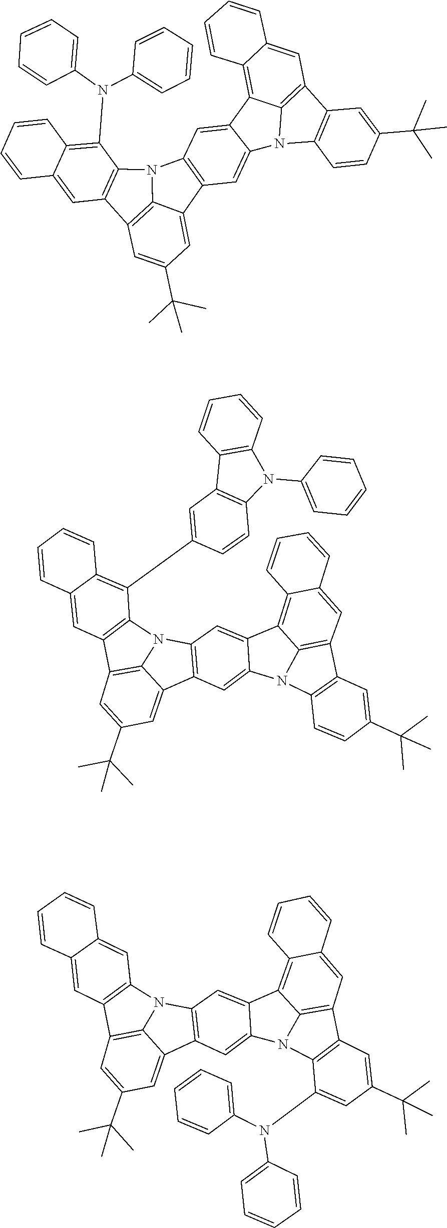 [SMILES: CC(C)(C)c1ccc2c(c1)c1cc3ccccc3c3c4cc5c(cc4n2c13)c1cc(C(C)(C)C)cc2c3cc4ccccc4c(N(c4ccccc4)c4ccccc4)c3n5c12 ; CC(C)(C)c1ccc2c(c1)c1cc3ccccc3c3c4cc5c(cc4n2c13)c1cc(C(C)(C)C)cc2c3cc4ccccc4c(-c4ccc6c(c4)c4ccccc4n6-c4ccccc4)c3n5c12 ; CC(C)(C)c1cc(N(c2ccccc2)c2ccccc2)c2c(c1)c1cc3ccccc3c3c4cc5c(cc4n2c13)c1cc(C(C)(C)C)cc2c3cc4ccccc4cc3n5c21]

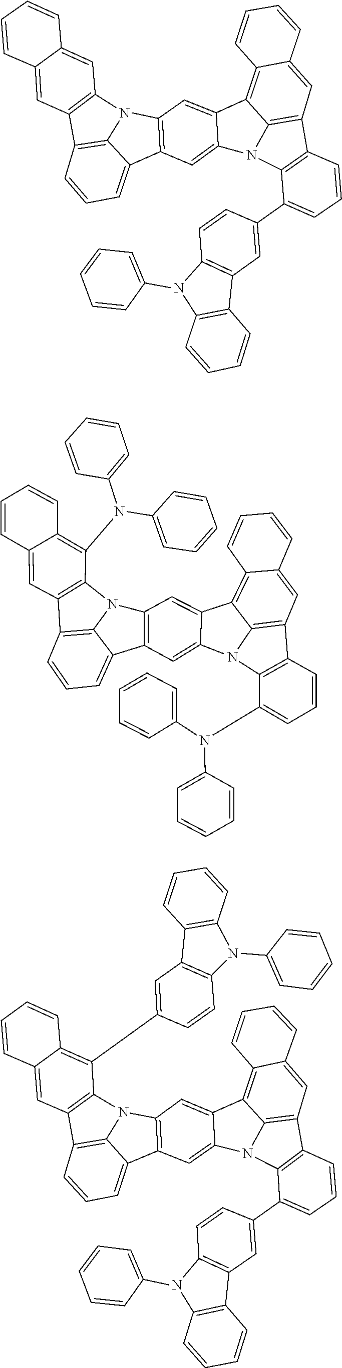

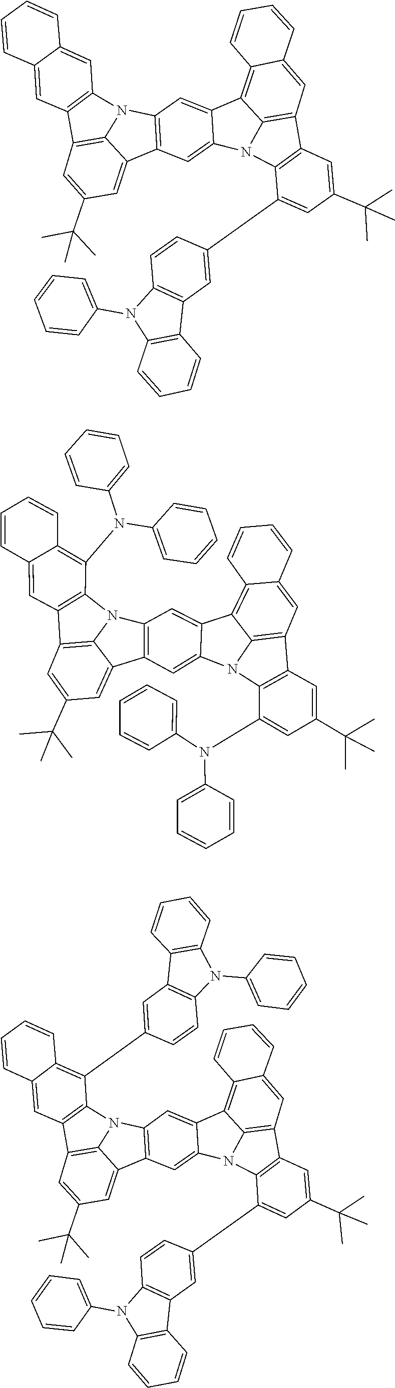 [SMILES: CC(C)(C)c1cc(-c2ccc3c(c2)c2ccccc2n3-c2ccccc2)c2c(c1)c1cc3ccccc3c3c4cc5c(cc4n2c13)c1cc(C(C)(C)C)cc2c3cc4ccccc4cc3n5c21 ; CC(C)(C)c1cc(N(c2ccccc2)c2ccccc2)c2c(c1)c1cc3ccccc3c3c4cc5c(cc4n2c13)c1cc(C(C)(C)C)cc2c3cc4ccccc4c(N(c4ccccc4)c4ccccc4)c3n5c12 ; CC(C)(C)c1cc(-c2ccc3c(c2)c2ccccc2n3-c2ccccc2)c2c(c1)c1cc3ccccc3c3c4cc5c(cc4n2c13)c1cc(C(C)(C)C)cc2c3cc4ccccc4c(-c4ccc6c(c4)c4ccccc4n6-c4ccccc4)c3n5c12]

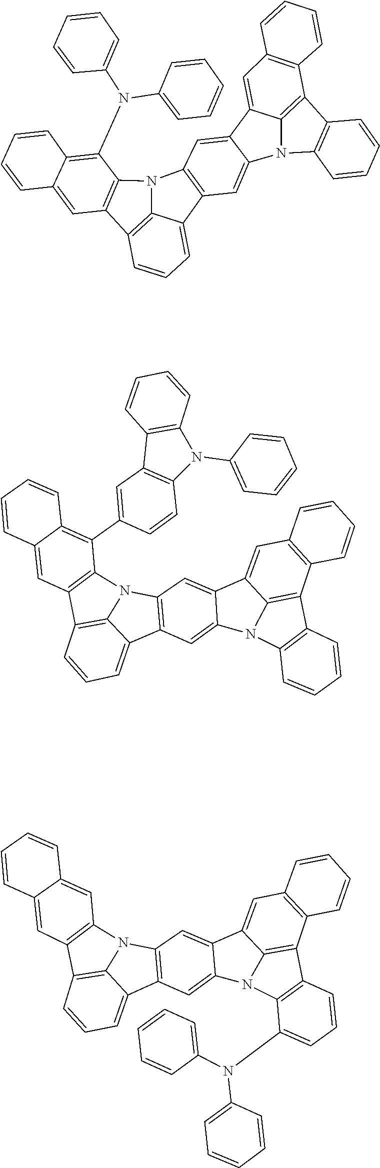 [SMILES: c1ccc(N(c2ccccc2)c2c3ccccc3cc3c4cccc5c6cc7c(cc6n(c23)c54)c2cc3ccccc3c3c4ccccc4n7c23)cc1 ; c1ccc(-n2c3ccccc3c3cc(-c4c5ccccc5cc5c6cccc7c8cc9c(cc8n(c45)c76)c4cc5ccccc5c5c6ccccc6n9c45)ccc32)cc1 ; c1ccc(N(c2ccccc2)c2cccc3c4c5ccccc5cc5c6cc7c(cc6n(c23)c54)c2cccc3c4cc5ccccc5cc4n7c32)cc1]

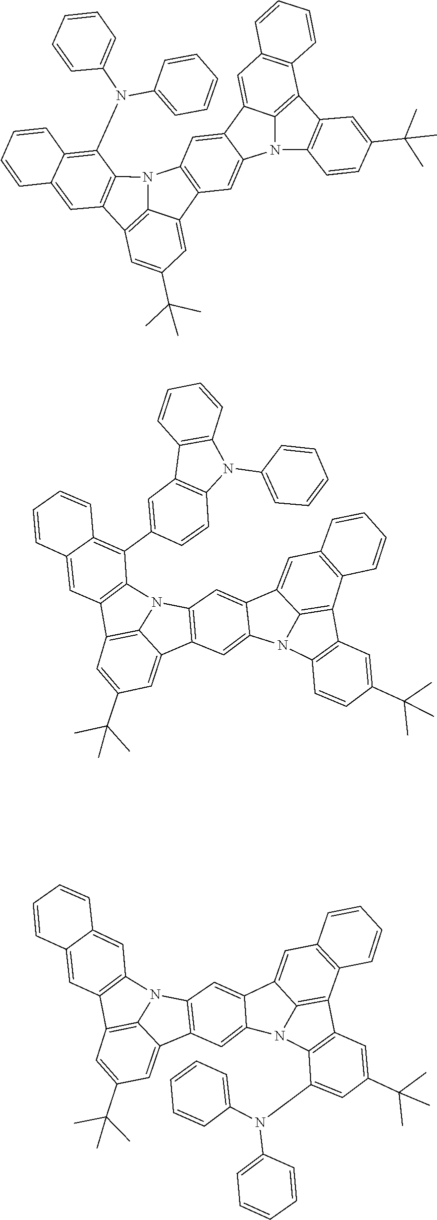 [SMILES: CC(C)(C)c1ccc2c(c1)c1c3ccccc3cc3c4cc5c(cc4n2c31)c1cc(C(C)(C)C)cc2c3cc4ccccc4c(N(c4ccccc4)c4ccccc4)c3n5c12 ; CC(C)(C)c1ccc2c(c1)c1c3ccccc3cc3c4cc5c(cc4n2c31)c1cc(C(C)(C)C)cc2c3cc4ccccc4c(-c4ccc6c(c4)c4ccccc4n6-c4ccccc4)c3n5c12 ; CC(C)(C)c1cc(N(c2ccccc2)c2ccccc2)c2c(c1)c1c3ccccc3cc3c4cc5c(cc4n2c31)c1cc(C(C)(C)C)cc2c3cc4ccccc4cc3n5c21]

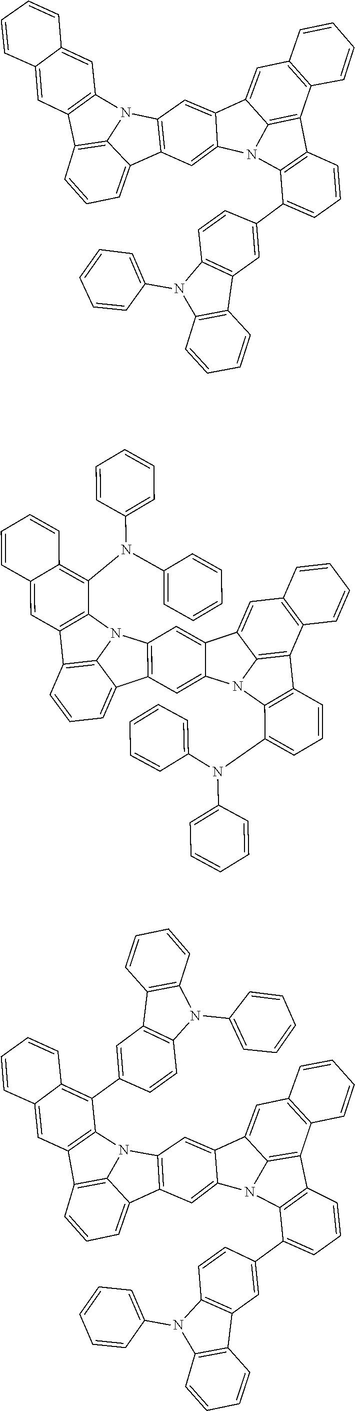 [SMILES: c1ccc(-n2c3ccccc3c3cc(-c4cccc5c6c7ccccc7cc7c8cc9c(cc8n(c45)c76)c4cccc5c6cc7ccccc7cc6n9c54)ccc32)cc1 ; c1ccc(N(c2ccccc2)c2c3ccccc3cc3c4cccc5c6cc7c(cc6n(c23)c54)c2cc3ccccc3c3c4cccc(N(c5ccccc5)c5ccccc5)c4n7c23)cc1 ; c1ccc(-n2c3ccccc3c3cc(-c4c5ccccc5cc5c6cccc7c8cc9c(cc8n(c45)c76)c4cc5ccccc5c5c6cccc(-c7ccc8c(c7)c7ccccc7n8-c7ccccc7)c6n9c45)ccc32)cc1]

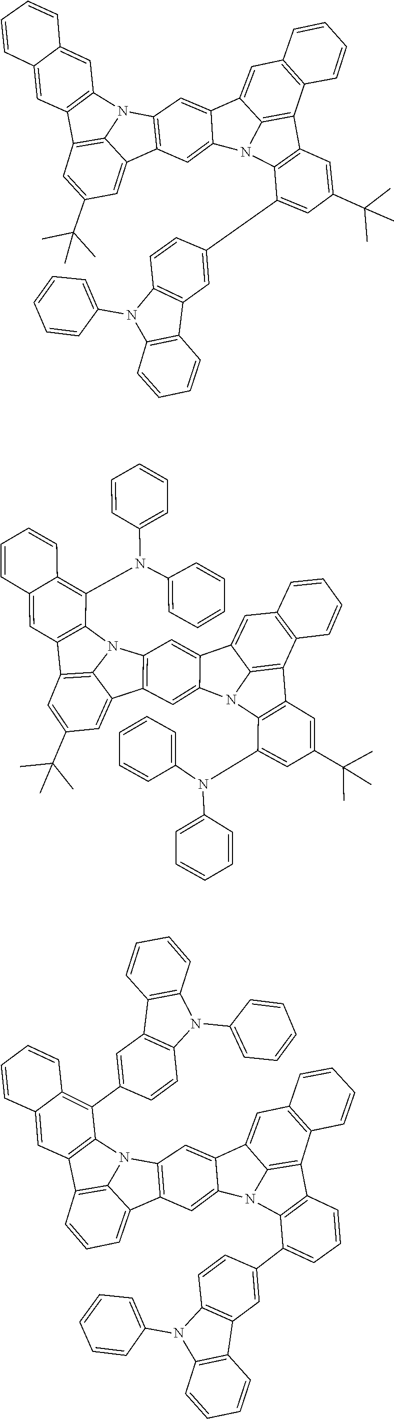 [SMILES: CC(C)(C)c1cc(-c2ccc3c(c2)c2ccccc2n3-c2ccccc2)c2c(c1)c1c3ccccc3cc3c4cc5c(cc4n2c31)c1cc(C(C)(C)C)cc2c3cc4ccccc4cc3n5c21 ; CC(C)(C)c1cc(N(c2ccccc2)c2ccccc2)c2c(c1)c1c3ccccc3cc3c4cc5c(cc4n2c31)c1cc(C(C)(C)C)cc2c3cc4ccccc4c(N(c4ccccc4)c4ccccc4)c3n5c12 ; c1ccc(-n2c3ccccc3c3cc(-c4c5ccccc5cc5c6cccc7c8cc9c(cc8n(c45)c76)c4cc5ccccc5c5c6cccc(-c7ccc8c(c7)c7ccccc7n8-c7ccccc7)c6n9c45)ccc32)cc1]

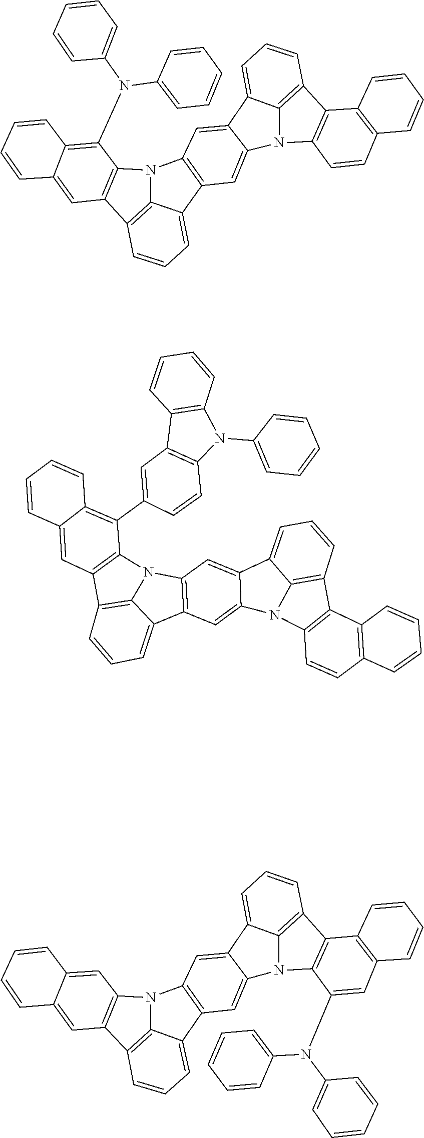 [SMILES: c1ccc(N(c2ccccc2)c2c3ccccc3cc3c4cccc5c6cc7c(cc6n(c23)c54)c2cccc3c4c5ccccc5ccc4n7c23)cc1 ; c1ccc(-n2c3ccccc3c3cc(-c4c5ccccc5cc5c6cccc7c8cc9c(cc8n(c45)c76)c4cccc5c6c7ccccc7ccc6n9c45)ccc32)cc1 ; c1ccc(N(c2ccccc2)c2cc3ccccc3c3c4cccc5c6cc7c(cc6n(c23)c54)c2cccc3c4cc5ccccc5cc4n7c32)cc1]

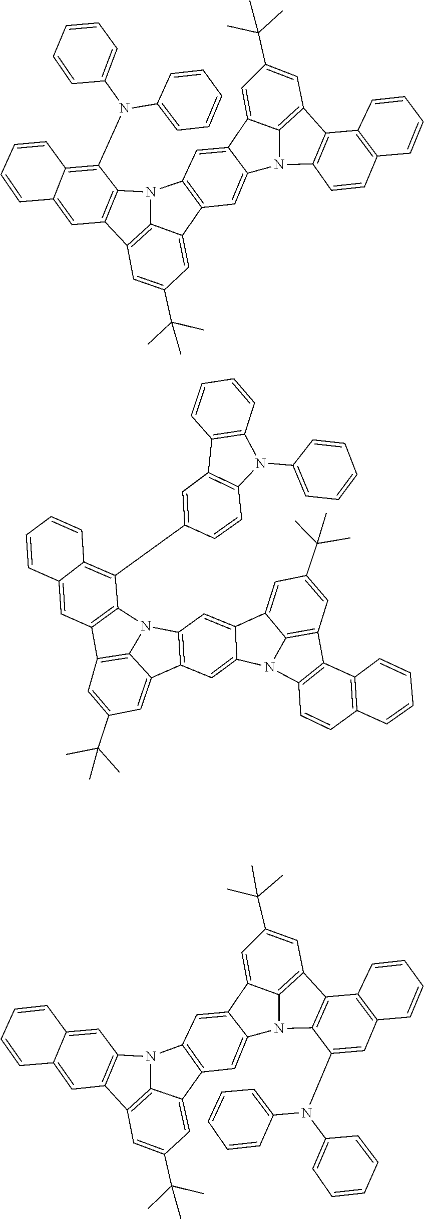

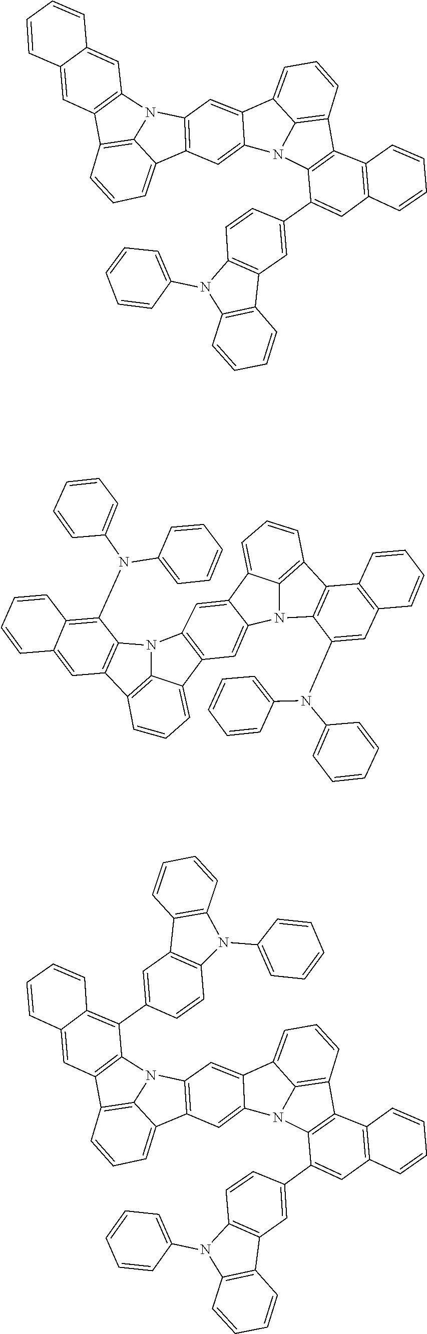 [SMILES: c1ccc(-n2c3ccccc3c3cc(-c4cc5ccccc5c5c6cccc7c8cc9c(cc8n(c45)c76)c4cccc5c6cc7ccccc7cc6n9c54)ccc32)cc1 ; c1ccc(N(c2ccccc2)c2c3ccccc3cc3c4cccc5c6cc7c(cc6n(c23)c54)c2cccc3c4c5ccccc5cc(N(c5ccccc5)c5ccccc5)c4n7c23)cc1 ; c1ccc(-n2c3ccccc3c3cc(-c4c5ccccc5cc5c6cccc7c8cc9c(cc8n(c45)c76)c4cccc5c6c7ccccc7cc(-c7ccc8c(c7)c7ccccc7n8-c7ccccc7)c6n9c45)ccc32)cc1]

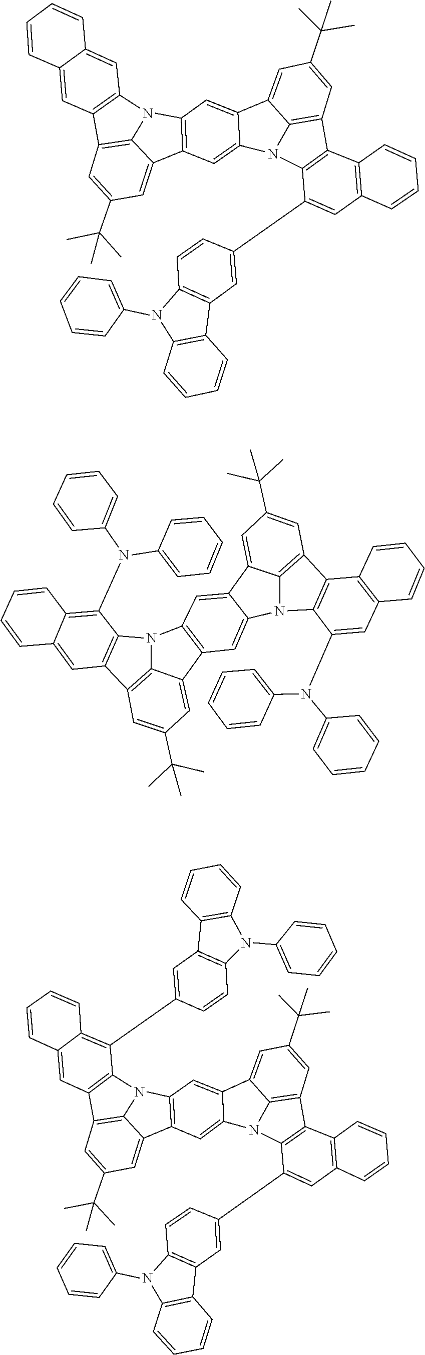 [SMILES: CC(C)(C)c1cc2c3cc4ccccc4cc3n3c4cc5c6cc(C(C)(C)C)cc7c8c9ccccc9cc(-c9ccc%10c(c9)c9ccccc9n%10-c9ccccc9)c8n(c5cc4c(c1)c23)c67 ; CC(C)(C)c1cc2c3cc4c(cc3n3c5c(N(c6ccccc6)c6ccccc6)c6ccccc6cc5c(c1)c23)c1cc(C(C)(C)C)cc2c3c5ccccc5cc(N(c5ccccc5)c5ccccc5)c3n4c12 ; CC(C)(C)c1cc2c3cc4c(cc3n3c5c(-c6ccc7c(c6)c6ccccc6n7-c6ccccc6)c6ccccc6cc5c(c1)c23)c1cc(C(C)(C)C)cc2c3c5ccccc5cc(-c5ccc6c(c5)c5ccccc5n6-c5ccccc5)c3n4c12]

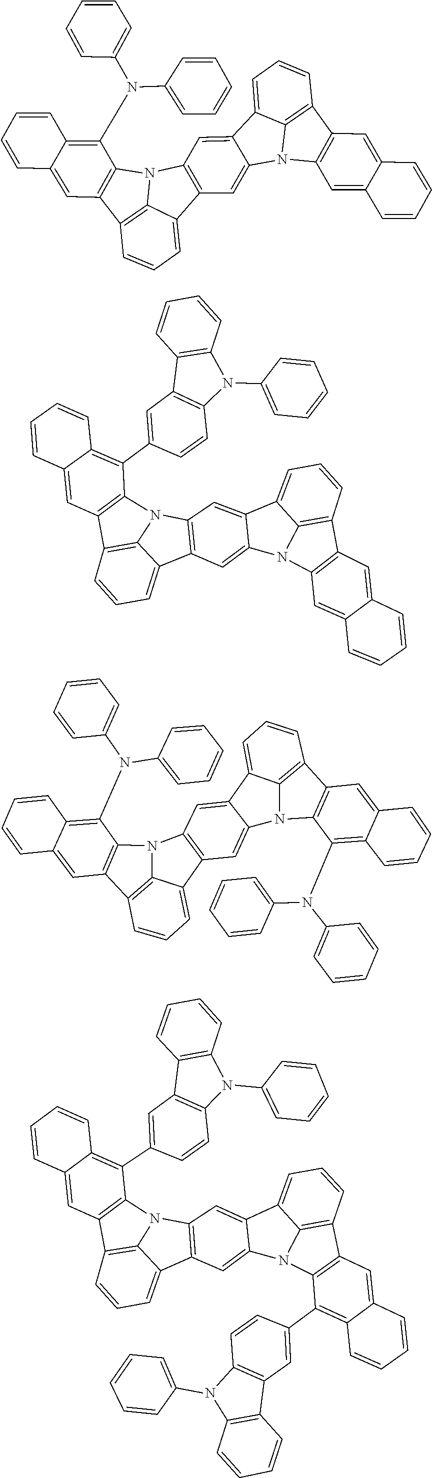

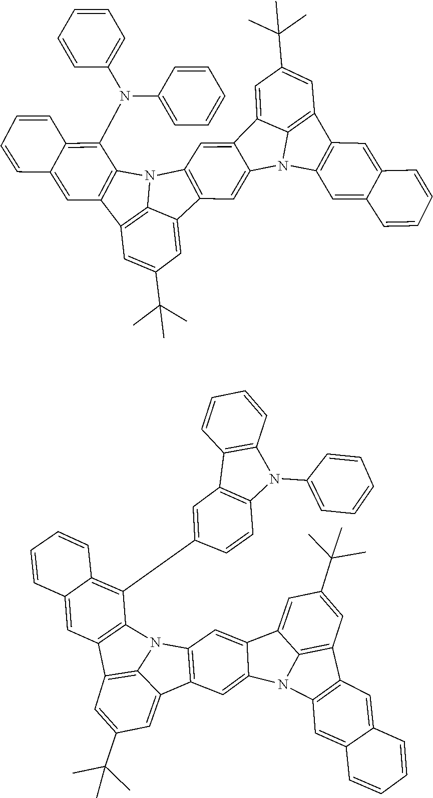 [SMILES: CC(C)(C)c1cc2c3cc4ccccc4cc3n3c4cc5c6cc(C(C)(C)C)cc7c8cc9ccccc9c(N(c9ccccc9)c9ccccc9)c8n(c5cc4c(c1)c23)c67 ; CC(C)(C)c1cc2c3cc4ccccc4cc3n3c4cc5c6cc(C(C)(C)C)cc7c8cc9ccccc9c(-c9ccc%10c(c9)c9ccccc9n%10-c9ccccc9)c8n(c5cc4c(c1)c23)c67]

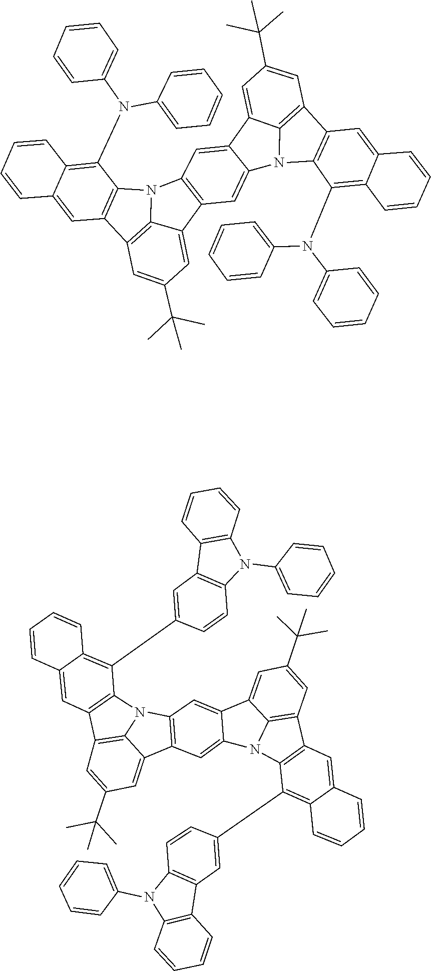 [SMILES: CC(C)(C)c1cc2c3cc4c(cc3n3c5c(N(c6ccccc6)c6ccccc6)c6ccccc6cc5c(c1)c23)c1cc(C(C)(C)C)cc2c3cc5ccccc5c(N(c5ccccc5)c5ccccc5)c3n4c12 ; CC(C)(C)c1cc2c3cc4c(cc3n3c5c(-c6ccc7c(c6)c6ccccc6n7-c6ccccc6)c6ccccc6cc5c(c1)c23)c1cc(C(C)(C)C)cc2c3cc5ccccc5c(-c5ccc6c(c5)c5ccccc5n6-c5ccccc5)c3n4c12]

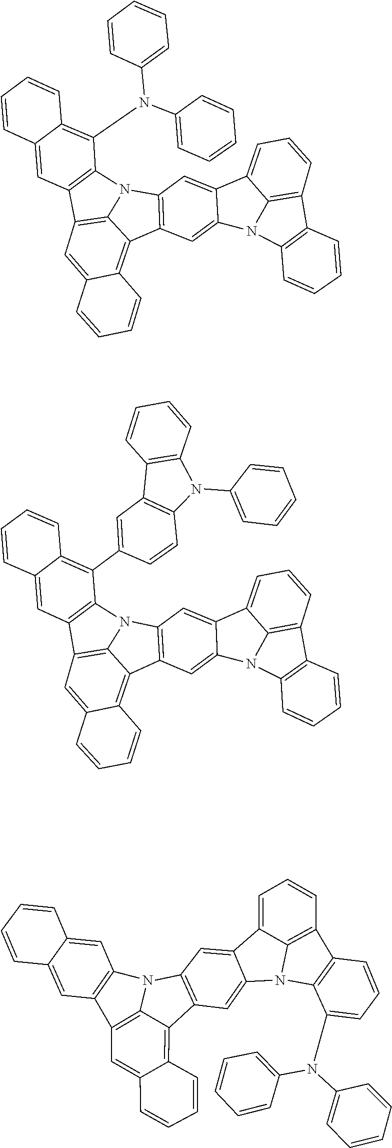 [SMILES: c1ccc(N(c2ccccc2)c2c3ccccc3cc3c4cc5ccccc5c5c6cc7c(cc6n(c23)c45)c2cccc3c4ccccc4n7c32)cc1 ; c1ccc(-n2c3ccccc3c3cc(-c4c5ccccc5cc5c6cc7ccccc7c7c8cc9c(cc8n(c45)c67)c4cccc5c6ccccc6n9c54)ccc32)cc1 ; c1ccc(N(c2ccccc2)c2cccc3c4cccc5c6cc7c(cc6n(c23)c54)c2c3ccccc3cc3c4cc5ccccc5cc4n7c32)cc1]

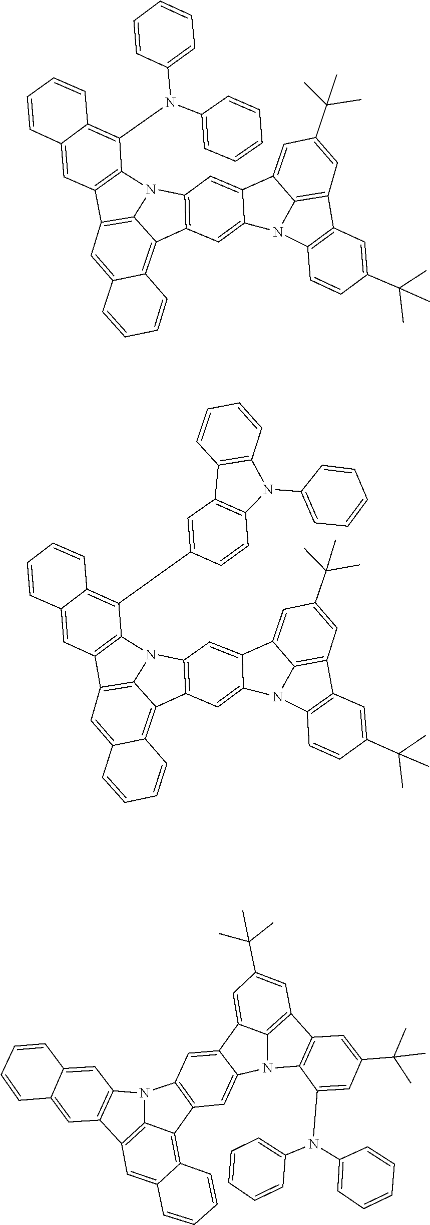 [SMILES: CC(C)(C)c1ccc2c(c1)c1cc(C(C)(C)C)cc3c4cc5c(cc4n2c13)c1c2ccccc2cc2c3cc4ccccc4c(N(c4ccccc4)c4ccccc4)c3n5c21 ; CC(C)(C)c1ccc2c(c1)c1cc(C(C)(C)C)cc3c4cc5c(cc4n2c13)c1c2ccccc2cc2c3cc4ccccc4c(-c4ccc6c(c4)c4ccccc4n6-c4ccccc4)c3n5c21 ; CC(C)(C)c1cc(N(c2ccccc2)c2ccccc2)c2c(c1)c1cc(C(C)(C)C)cc3c4cc5c(cc4n2c31)c1c2ccccc2cc2c3cc4ccccc4cc3n5c21]

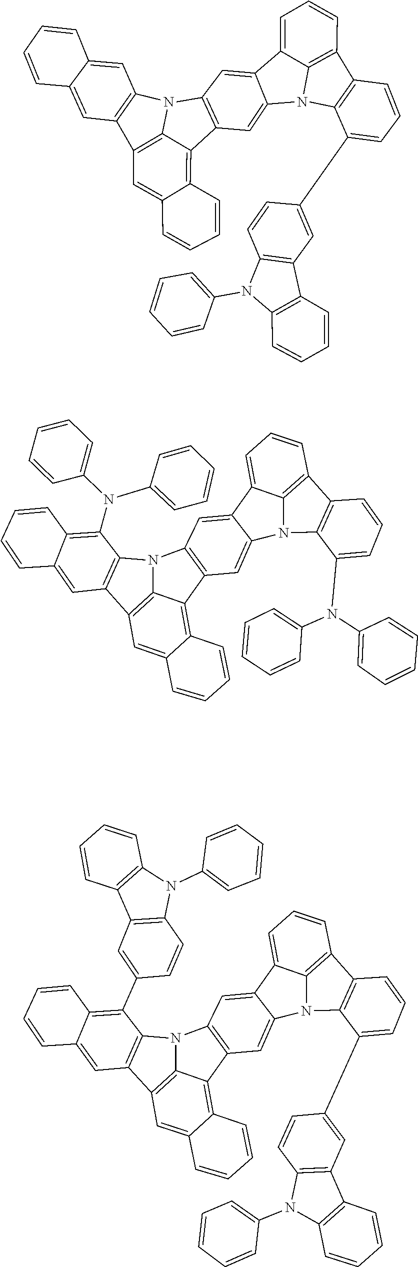 [SMILES: c1ccc(-n2c3ccccc3c3cc(-c4cccc5c6cccc7c8cc9c(cc8n(c45)c76)c4c5ccccc5cc5c6cc7ccccc7cc6n9c54)ccc32)cc1 ; c1ccc(N(c2ccccc2)c2cccc3c4cccc5c6cc7c(cc6n(c23)c54)c2c3ccccc3cc3c4cc5ccccc5c(N(c5ccccc5)c5ccccc5)c4n7c32)cc1 ; c1ccc(-n2c3ccccc3c3cc(-c4cccc5c6cccc7c8cc9c(cc8n(c45)c76)c4c5ccccc5cc5c6cc7ccccc7c(-c7ccc8c(c7)c7ccccc7n8-c7ccccc7)c6n9c54)ccc32)cc1]

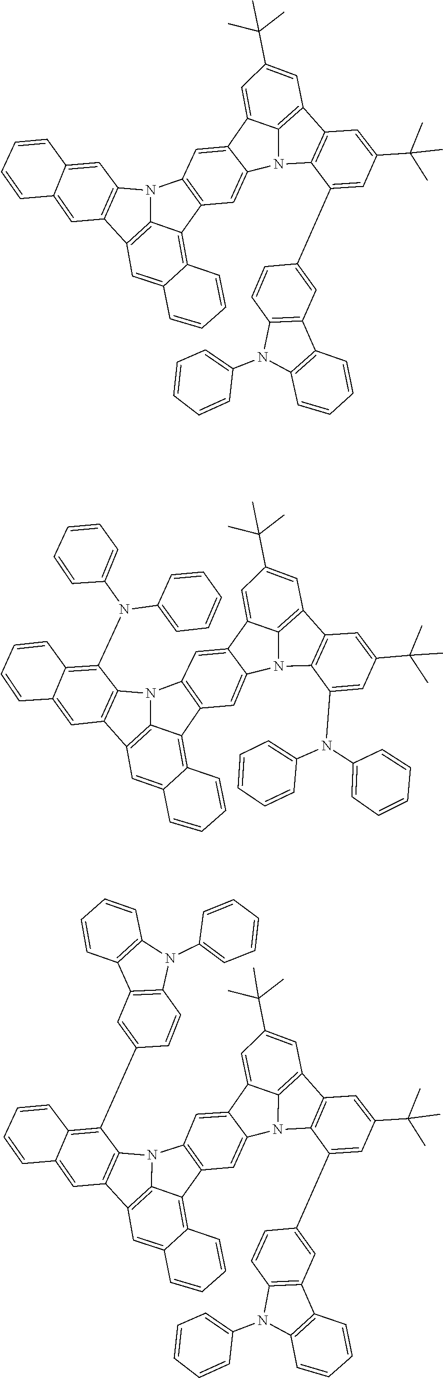 [SMILES: CC(C)(C)c1cc(-c2ccc3c(c2)c2ccccc2n3-c2ccccc2)c2c(c1)c1cc(C(C)(C)C)cc3c4cc5c(cc4n2c31)c1c2ccccc2cc2c3cc4ccccc4cc3n5c21 ; CC(C)(C)c1cc(N(c2ccccc2)c2ccccc2)c2c(c1)c1cc(C(C)(C)C)cc3c4cc5c(cc4n2c31)c1c2ccccc2cc2c3cc4ccccc4c(N(c4ccccc4)c4ccccc4)c3n5c21 ; CC(C)(C)c1cc(-c2ccc3c(c2)c2ccccc2n3-c2ccccc2)c2c(c1)c1cc(C(C)(C)C)cc3c4cc5c(cc4n2c31)c1c2ccccc2cc2c3cc4ccccc4c(-c4ccc6c(c4)c4ccccc4n6-c4ccccc4)c3n5c21]

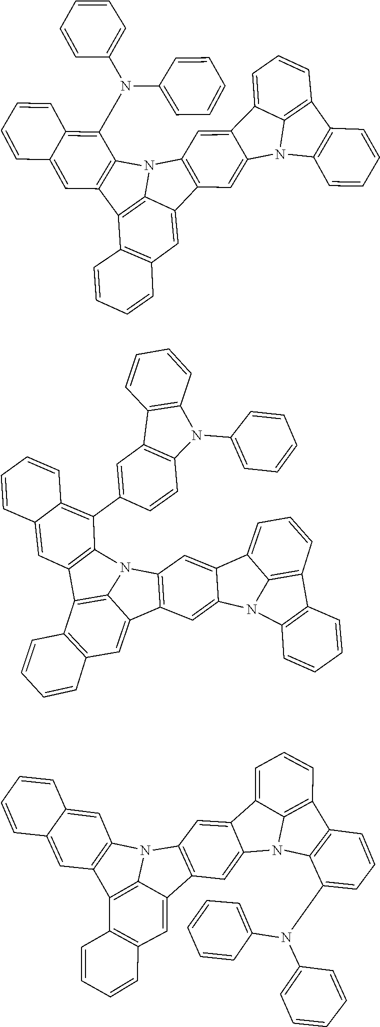

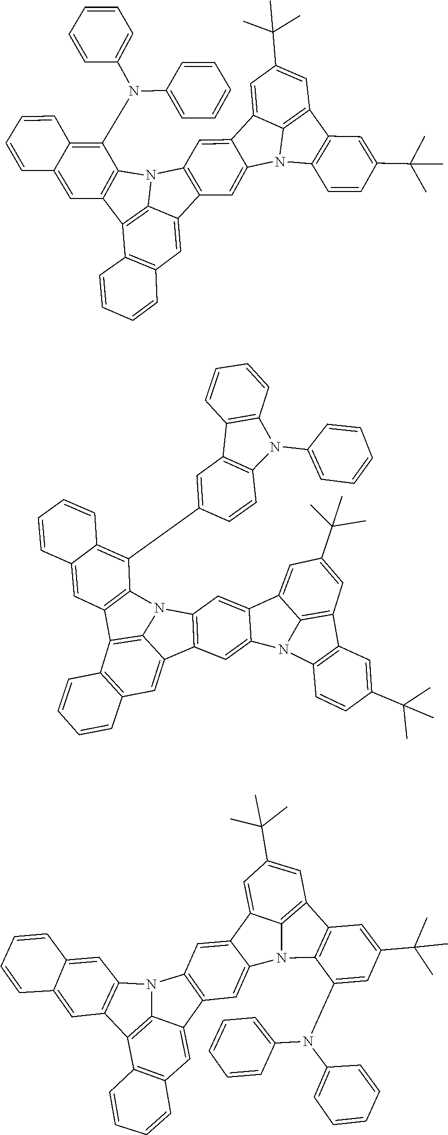 [SMILES: CC(C)(C)c1ccc2c(c1)c1cc(C(C)(C)C)cc3c4cc5c(cc4n2c13)c1cc2ccccc2c2c3cc4ccccc4c(N(c4ccccc4)c4ccccc4)c3n5c12 ; CC(C)(C)c1ccc2c(c1)c1cc(C(C)(C)C)cc3c4cc5c(cc4n2c13)c1cc2ccccc2c2c3cc4ccccc4c(-c4ccc6c(c4)c4ccccc4n6-c4ccccc4)c3n5c12 ; CC(C)(C)c1cc(N(c2ccccc2)c2ccccc2)c2c(c1)c1cc(C(C)(C)C)cc3c4cc5c(cc4n2c31)c1cc2ccccc2c2c3cc4ccccc4cc3n5c12]

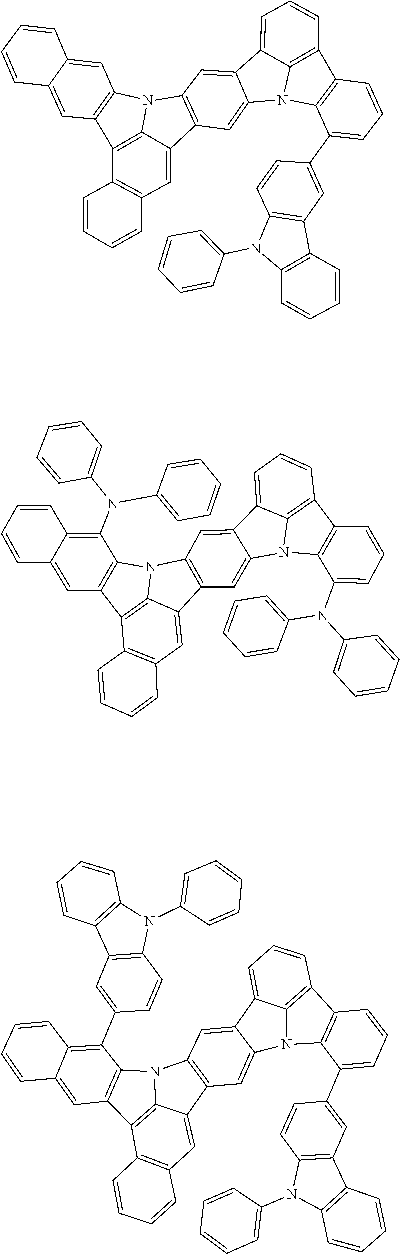 [SMILES: c1ccc(-n2c3ccccc3c3cc(-c4cccc5c6cccc7c8cc9c(cc8n(c45)c76)c4cc5ccccc5c5c6cc7ccccc7cc6n9c45)ccc32)cc1 ; c1ccc(N(c2ccccc2)c2cccc3c4cccc5c6cc7c(cc6n(c23)c54)c2cc3ccccc3c3c4cc5ccccc5c(N(c5ccccc5)c5ccccc5)c4n7c23)cc1 ; c1ccc(-n2c3ccccc3c3cc(-c4cccc5c6cccc7c8cc9c(cc8n(c45)c76)c4cc5ccccc5c5c6cc7ccccc7c(-c7ccc8c(c7)c7ccccc7n8-c7ccccc7)c6n9c45)ccc32)cc1]

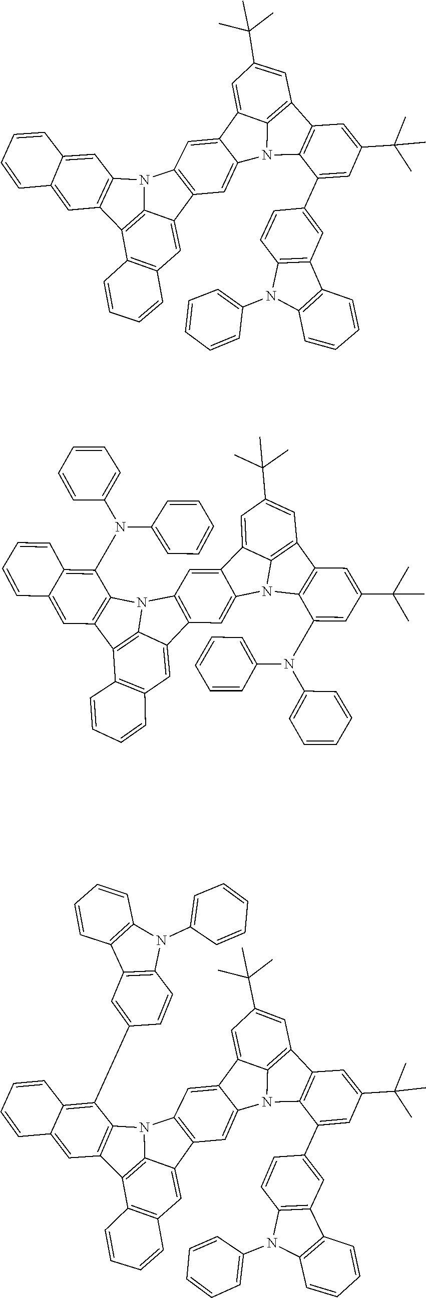 [SMILES: CC(C)(C)c1cc(-c2ccc3c(c2)c2ccccc2n3-c2ccccc2)c2c(c1)c1cc(C(C)(C)C)cc3c4cc5c(cc4n2c31)c1cc2ccccc2c2c3cc4ccccc4cc3n5c12 ; CC(C)(C)c1cc(N(c2ccccc2)c2ccccc2)c2c(c1)c1cc(C(C)(C)C)cc3c4cc5c(cc4n2c31)c1cc2ccccc2c2c3cc4ccccc4c(N(c4ccccc4)c4ccccc4)c3n5c12 ; CC(C)(C)c1cc(-c2ccc3c(c2)c2ccccc2n3-c2ccccc2)c2c(c1)c1cc(C(C)(C)C)cc3c4cc5c(cc4n2c31)c1cc2ccccc2c2c3cc4ccccc4c(-c4ccc6c(c4)c4ccccc4n6-c4ccccc4)c3n5c12]

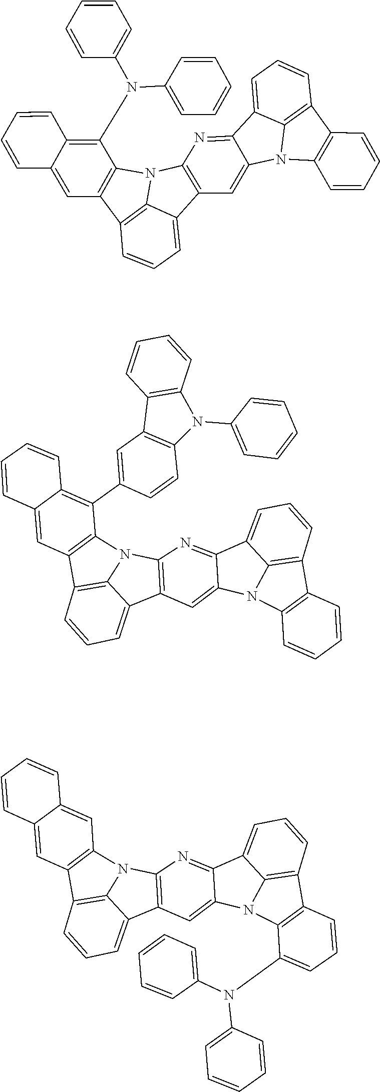 [SMILES: c1ccc(N(c2ccccc2)c2c3ccccc3cc3c4cccc5c6cc7c(nc6n(c23)c54)c2cccc3c4ccccc4n7c32)cc1 ; c1ccc(-n2c3ccccc3c3cc(-c4c5ccccc5cc5c6cccc7c8cc9c(nc8n(c45)c76)c4cccc5c6ccccc6n9c54)ccc32)cc1 ; c1ccc(N(c2ccccc2)c2cccc3c4cccc5c6nc7c(cc6n(c23)c45)c2cccc3c4cc5ccccc5cc4n7c32)cc1]

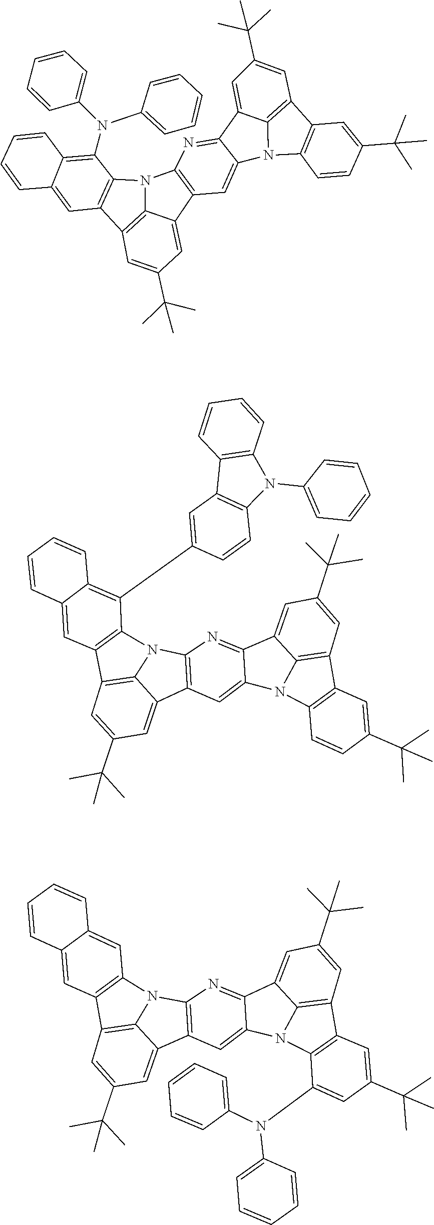 [SMILES: CC(C)(C)c1ccc2c(c1)c1cc(C(C)(C)C)cc3c4nc5c(cc4n2c13)c1cc(C(C)(C)C)cc2c3cc4ccccc4c(N(c4ccccc4)c4ccccc4)c3n5c12 ; CC(C)(C)c1ccc2c(c1)c1cc(C(C)(C)C)cc3c4nc5c(cc4n2c13)c1cc(C(C)(C)C)cc2c3cc4ccccc4c(-c4ccc6c(c4)c4ccccc4n6-c4ccccc4)c3n5c12 ; CC(C)(C)c1cc(N(c2ccccc2)c2ccccc2)c2c(c1)c1cc(C(C)(C)C)cc3c4nc5c(cc4n2c13)c1cc(C(C)(C)C)cc2c3cc4ccccc4cc3n5c21]

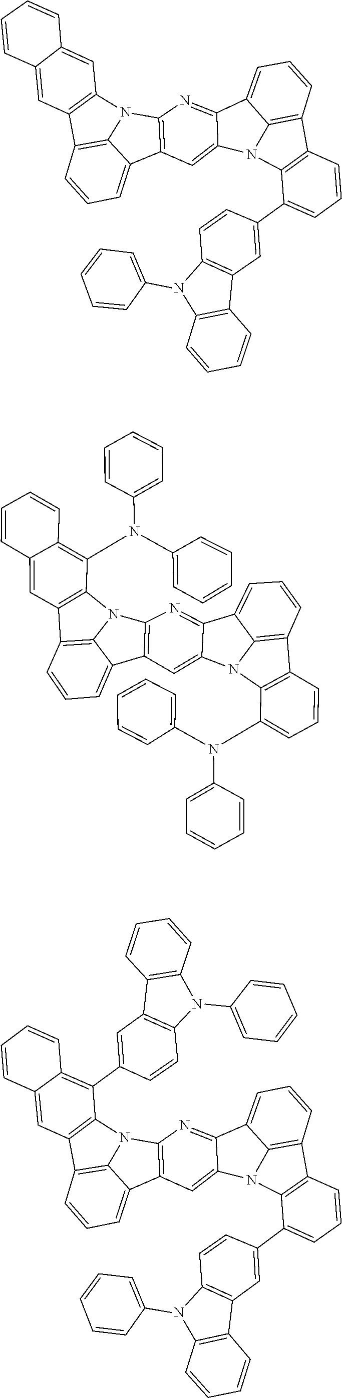 [SMILES: c1ccc(-n2c3ccccc3c3cc(-c4cccc5c6cccc7c8nc9c(cc8n(c45)c67)c4cccc5c6cc7ccccc7cc6n9c54)ccc32)cc1 ; c1ccc(N(c2ccccc2)c2cccc3c4cccc5c6nc7c(cc6n(c23)c45)c2cccc3c4cc5ccccc5c(N(c5ccccc5)c5ccccc5)c4n7c23)cc1 ; c1ccc(-n2c3ccccc3c3cc(-c4cccc5c6cccc7c8nc9c(cc8n(c45)c67)c4cccc5c6cc7ccccc7c(-c7ccc8c(c7)c7ccccc7n8-c7ccccc7)c6n9c45)ccc32)cc1]

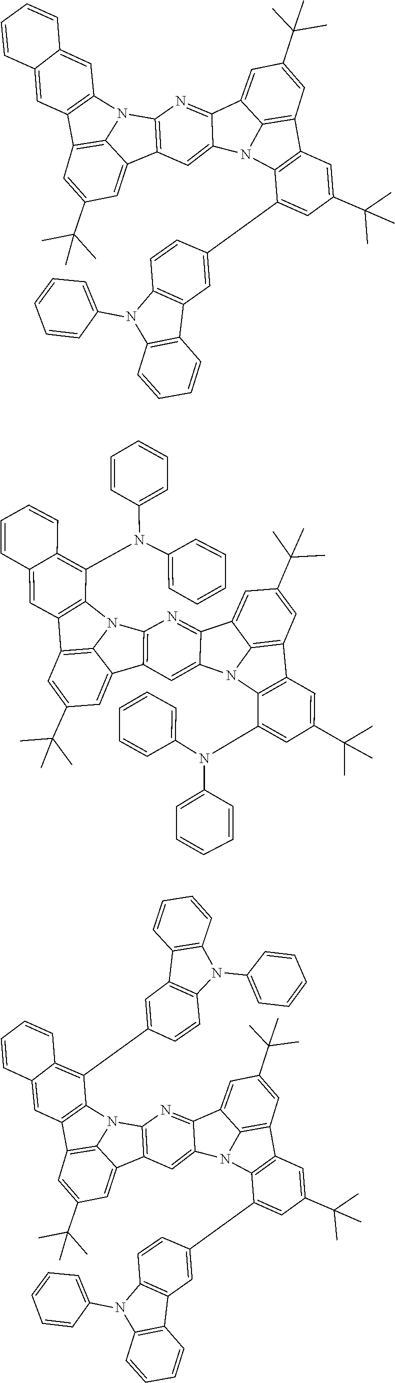

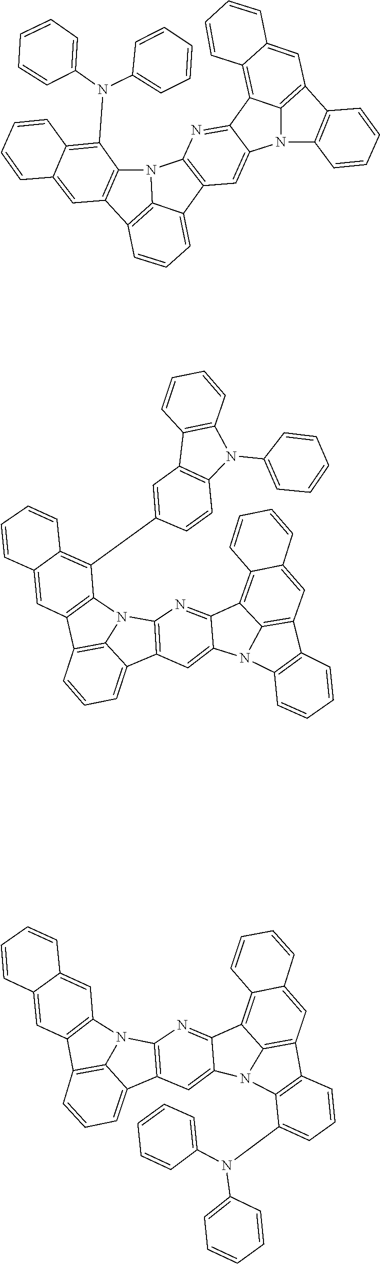 [SMILES: c1ccc(N(c2ccccc2)c2c3ccccc3cc3c4cccc5c6cc7c(nc6n(c23)c54)c2c3ccccc3cc3c4ccccc4n7c32)cc1 ; c1ccc(-n2c3ccccc3c3cc(-c4c5ccccc5cc5c6cccc7c8cc9c(nc8n(c45)c76)c4c5ccccc5cc5c6ccccc6n9c54)ccc32)cc1 ; c1ccc(N(c2ccccc2)c2cccc3c4cc5ccccc5c5c6nc7c(cc6n(c23)c45)c2cccc3c4cc5ccccc5cc4n7c32)cc1]

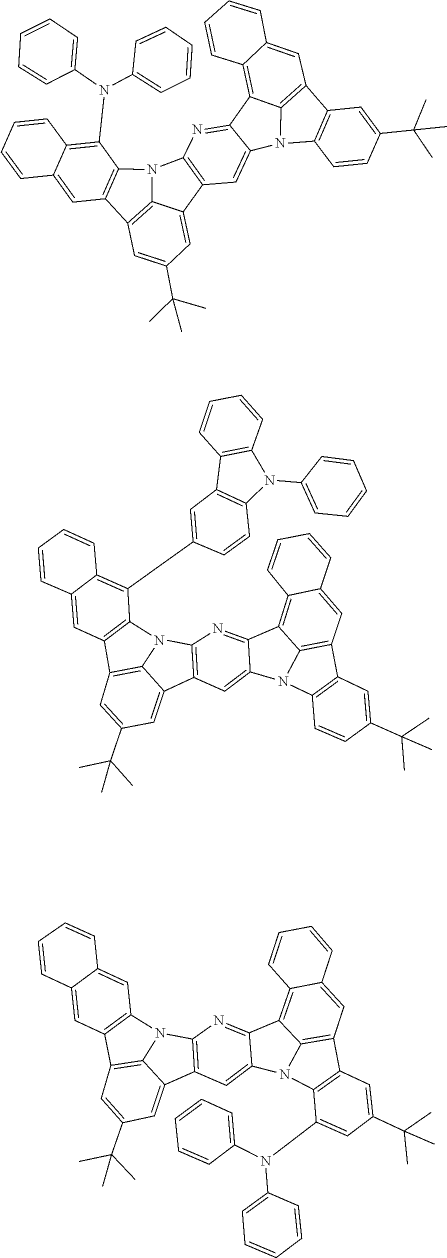 [SMILES: CC(C)(C)c1ccc2c(c1)c1cc3ccccc3c3c4nc5c(cc4n2c13)c1cc(C(C)(C)C)cc2c3cc4ccccc4c(N(c4ccccc4)c4ccccc4)c3n5c12 ; CC(C)(C)c1ccc2c(c1)c1cc3ccccc3c3c4nc5c(cc4n2c13)c1cc(C(C)(C)C)cc2c3cc4ccccc4c(-c4ccc6c(c4)c4ccccc4n6-c4ccccc4)c3n5c12 ; CC(C)(C)c1cc(N(c2ccccc2)c2ccccc2)c2c(c1)c1cc3ccccc3c3c4nc5c(cc4n2c13)c1cc(C(C)(C)C)cc2c3cc4ccccc4cc3n5c21]

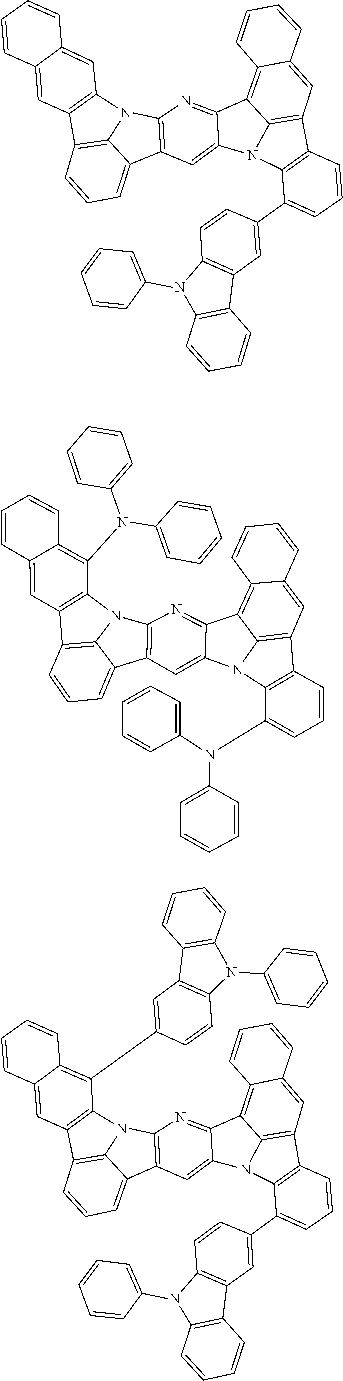

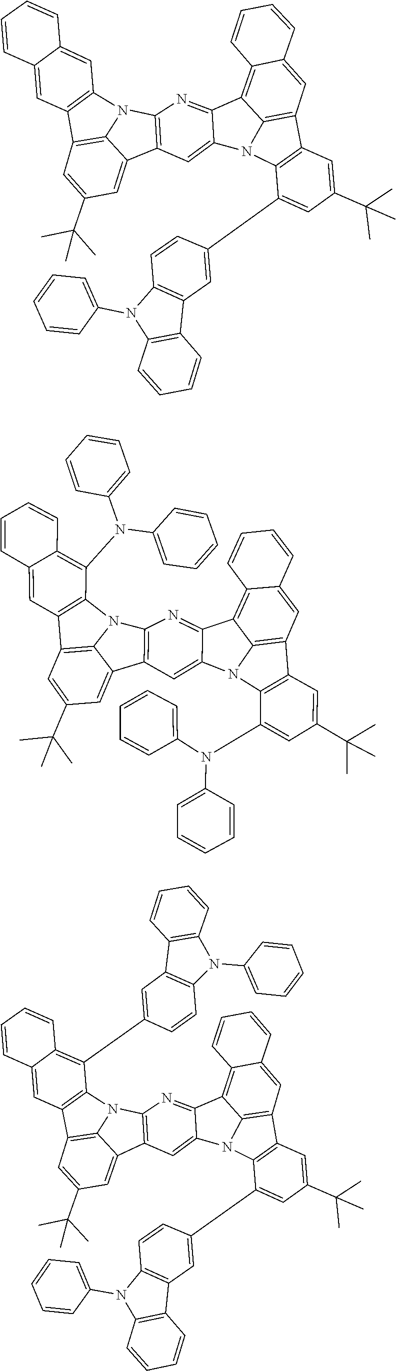

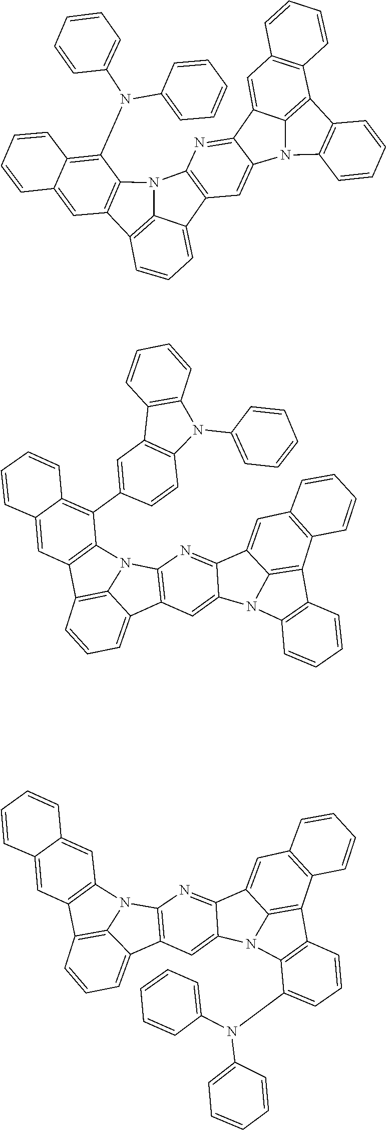 [SMILES: c1ccc(N(c2ccccc2)c2c3ccccc3cc3c4cccc5c6cc7c(nc6n(c23)c54)c2cc3ccccc3c3c4ccccc4n7c23)cc1 ; c1ccc(-n2c3ccccc3c3cc(-c4c5ccccc5cc5c6cccc7c8cc9c(nc8n(c45)c76)c4cc5ccccc5c5c6ccccc6n9c45)ccc32)cc1 ; c1ccc(N(c2ccccc2)c2cccc3c4c5ccccc5cc5c6nc7c(cc6n(c23)c54)c2cccc3c4cc5ccccc5cc4n7c32)cc1]

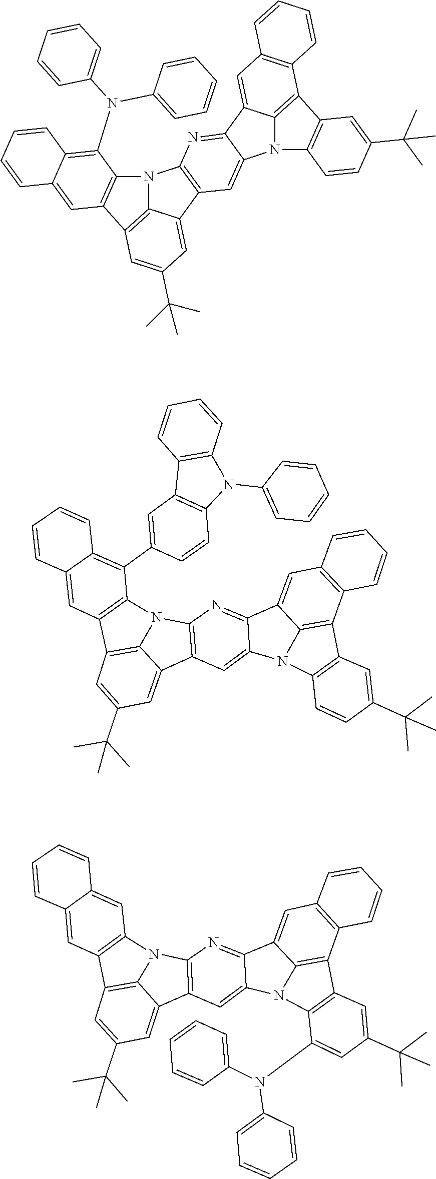 [SMILES: CC(C)(C)c1ccc2c(c1)c1c3ccccc3cc3c4nc5c(cc4n2c31)c1cc(C(C)(C)C)cc2c3cc4ccccc4c(N(c4ccccc4)c4ccccc4)c3n5c12 ; CC(C)(C)c1ccc2c(c1)c1c3ccccc3cc3c4nc5c(cc4n2c31)c1cc(C(C)(C)C)cc2c3cc4ccccc4c(-c4ccc6c(c4)c4ccccc4n6-c4ccccc4)c3n5c12 ; CC(C)(C)c1cc(N(c2ccccc2)c2ccccc2)c2c(c1)c1c3ccccc3cc3c4nc5c(cc4n2c31)c1cc(C(C)(C)C)cc2c3cc4ccccc4cc3n5c21]

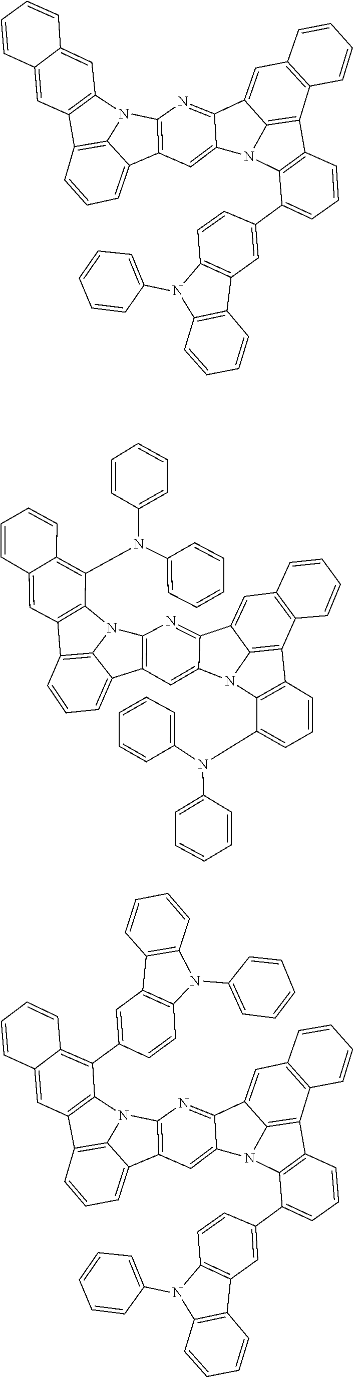 [SMILES: c1ccc(-n2c3ccccc3c3cc(-c4cccc5c6c7ccccc7cc7c8nc9c(cc8n(c45)c76)c4cccc5c6cc7ccccc7cc6n9c54)ccc32)cc1 ; c1ccc(N(c2ccccc2)c2cccc3c4c5ccccc5cc5c6nc7c(cc6n(c23)c54)c2cccc3c4cc5ccccc5c(N(c5ccccc5)c5ccccc5)c4n7c23)cc1 ; c1ccc(-n2c3ccccc3c3cc(-c4cccc5c6c7ccccc7cc7c8nc9c(cc8n(c45)c76)c4cccc5c6cc7ccccc7c(-c7ccc8c(c7)c7ccccc7n8-c7ccccc7)c6n9c45)ccc32)cc1]

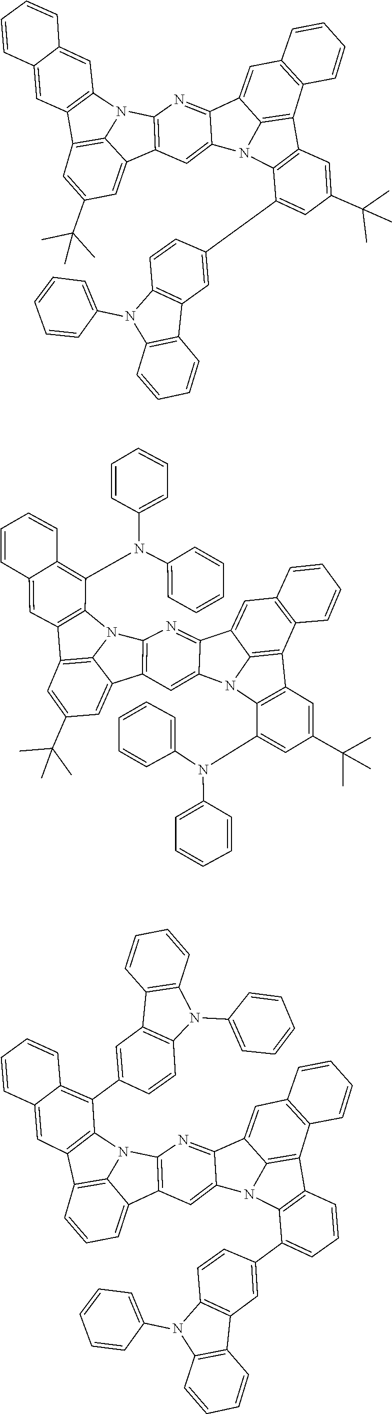 [SMILES: CC(C)(C)c1cc(-c2ccc3c(c2)c2ccccc2n3-c2ccccc2)c2c(c1)c1c3ccccc3cc3c4nc5c(cc4n2c31)c1cc(C(C)(C)C)cc2c3cc4ccccc4cc3n5c21 ; CC(C)(C)c1cc(N(c2ccccc2)c2ccccc2)c2c(c1)c1c3ccccc3cc3c4nc5c(cc4n2c31)c1cc(C(C)(C)C)cc2c3cc4ccccc4c(N(c4ccccc4)c4ccccc4)c3n5c12 ; c1ccc(-n2c3ccccc3c3cc(-c4cccc5c6c7ccccc7cc7c8nc9c(cc8n(c45)c76)c4cccc5c6cc7ccccc7c(-c7ccc8c(c7)c7ccccc7n8-c7ccccc7)c6n9c45)ccc32)cc1]

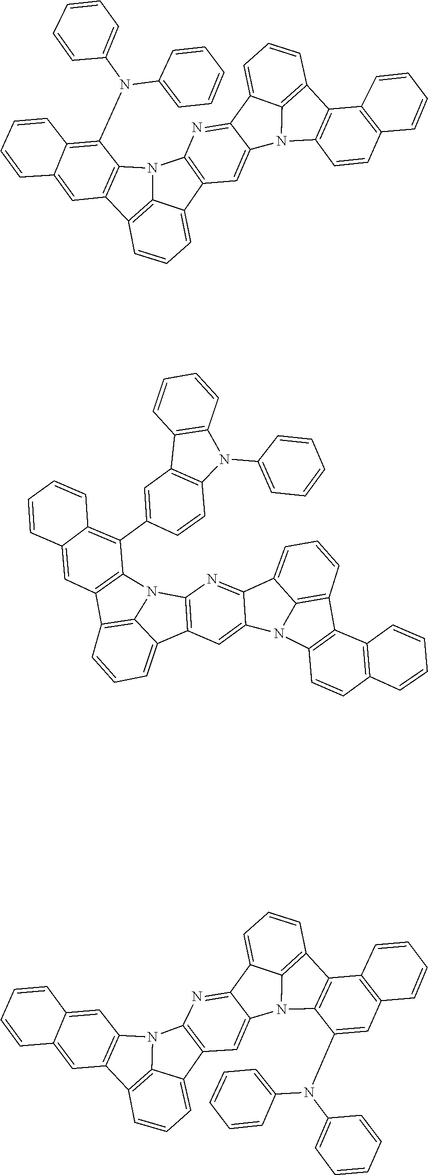 [SMILES: c1ccc(N(c2ccccc2)c2c3ccccc3cc3c4cccc5c6cc7c(nc6n(c23)c54)c2cccc3c4c5ccccc5ccc4n7c23)cc1 ; c1ccc(-n2c3ccccc3c3cc(-c4c5ccccc5cc5c6cccc7c8cc9c(nc8n(c45)c76)c4cccc5c6c7ccccc7ccc6n9c45)ccc32)cc1 ; c1ccc(N(c2ccccc2)c2cc3ccccc3c3c4cccc5c6nc7c(cc6n(c23)c54)c2cccc3c4cc5ccccc5cc4n7c32)cc1]

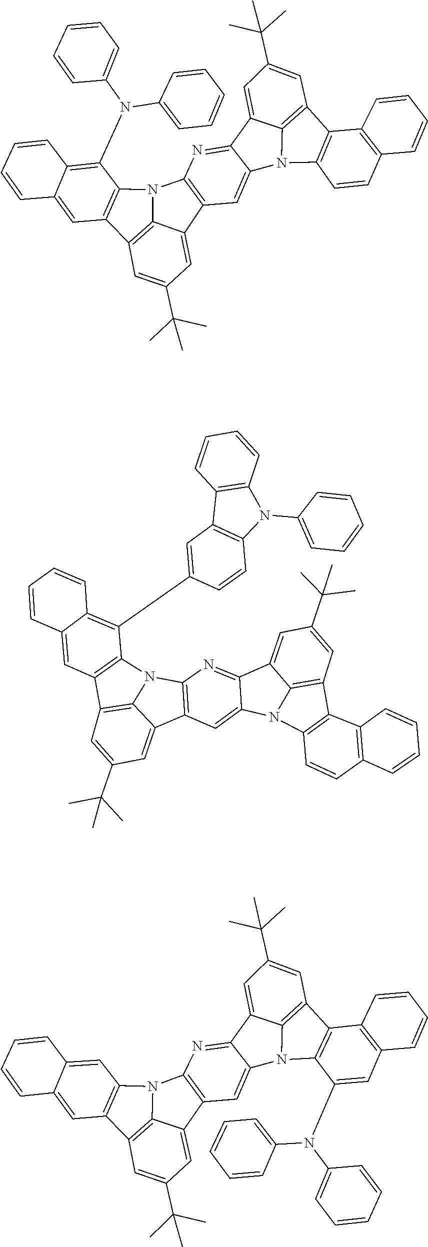 [SMILES: CC(C)(C)c1cc2c3nc4c(cc3n3c5ccc6ccccc6c5c(c1)c23)c1cc(C(C)(C)C)cc2c3cc5ccccc5c(N(c5ccccc5)c5ccccc5)c3n4c12 ; CC(C)(C)c1cc2c3nc4c(cc3n3c5ccc6ccccc6c5c(c1)c23)c1cc(C(C)(C)C)cc2c3cc5ccccc5c(-c5ccc6c(c5)c5ccccc5n6-c5ccccc5)c3n4c12 ; CC(C)(C)c1cc2c3cc4ccccc4cc3n3c4nc5c6cc(C(C)(C)C)cc7c8c9ccccc9cc(N(c9ccccc9)c9ccccc9)c8n(c5cc4c(c1)c23)c67]

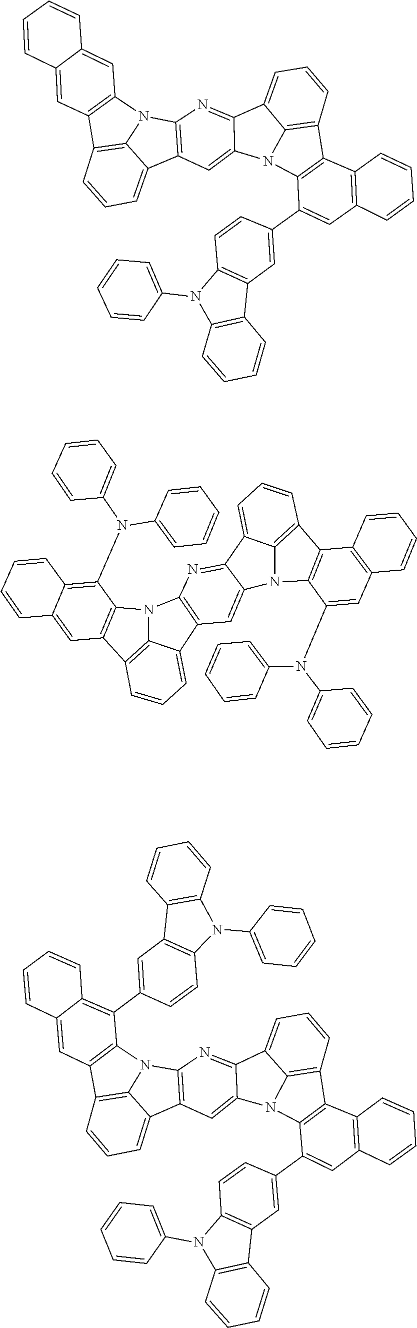 [SMILES: c1ccc(-n2c3ccccc3c3cc(-c4cc5ccccc5c5c6cccc7c8nc9c(cc8n(c45)c76)c4cccc5c6cc7ccccc7cc6n9c54)ccc32)cc1 ; c1ccc(N(c2ccccc2)c2cc3ccccc3c3c4cccc5c6nc7c(cc6n(c23)c54)c2cccc3c4cc5ccccc5c(N(c5ccccc5)c5ccccc5)c4n7c23)cc1 ; c1ccc(-n2c3ccccc3c3cc(-c4cc5ccccc5c5c6cccc7c8nc9c(cc8n(c45)c76)c4cccc5c6cc7ccccc7c(-c7ccc8c(c7)c7ccccc7n8-c7ccccc7)c6n9c45)ccc32)cc1]

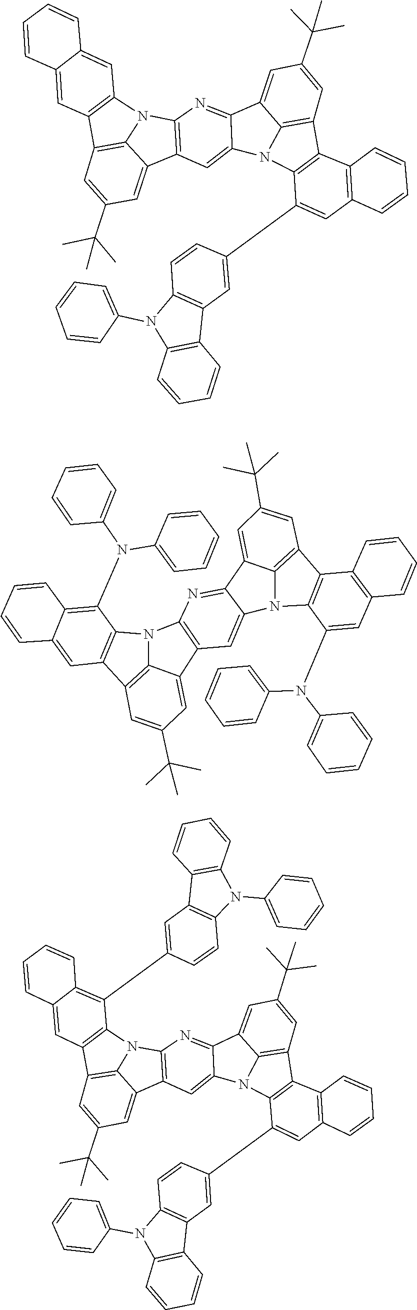

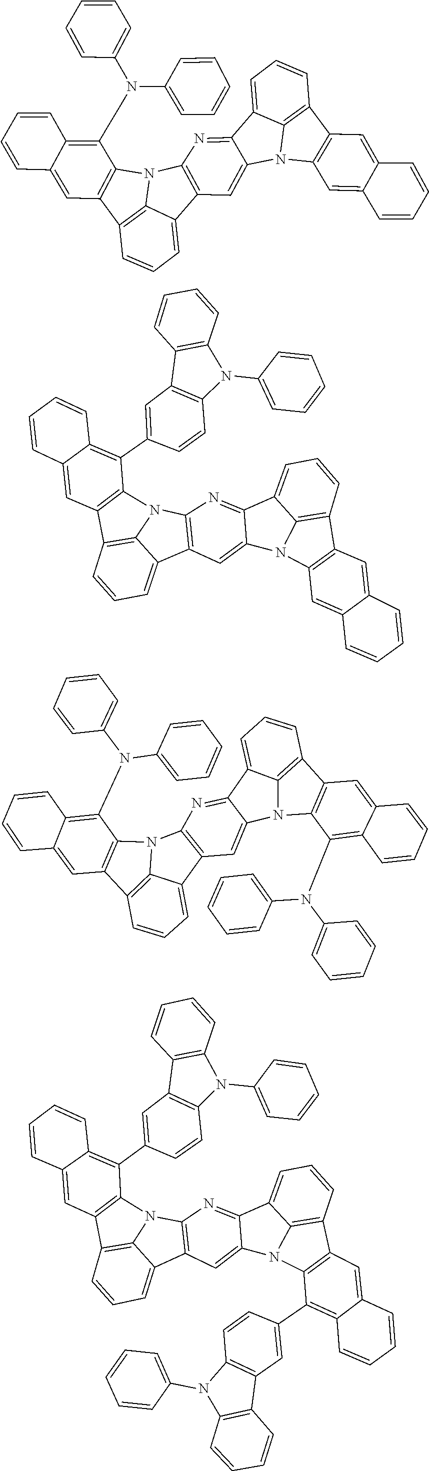

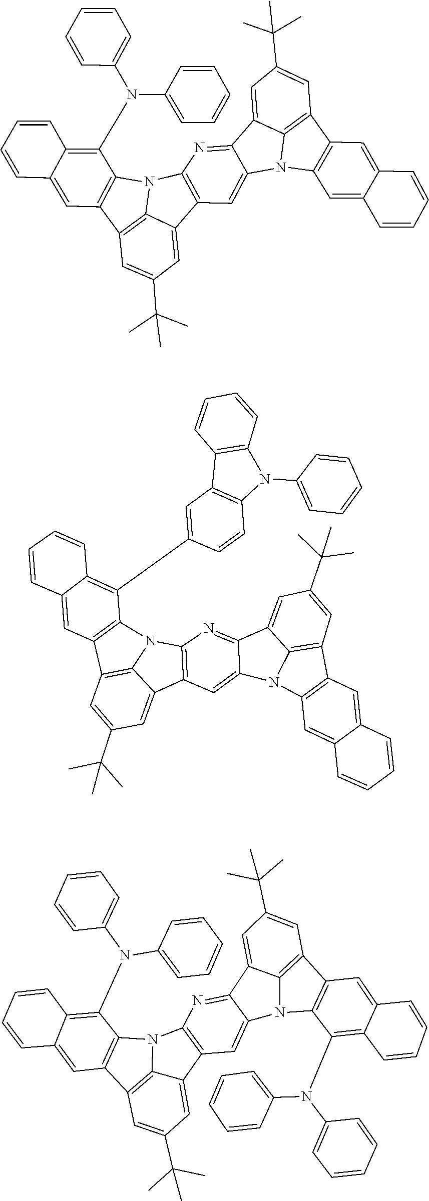 [SMILES: CC(C)(C)c1cc2c3cc4ccccc4cc3n3c4cc5c6cc(C(C)(C)C)cc7c8cc9ccccc9c(N(c9ccccc9)c9ccccc9)c8n(c5nc4c(c1)c23)c67 ; CC(C)(C)c1cc2c3cc4ccccc4cc3n3c4cc5c6cc(C(C)(C)C)cc7c8cc9ccccc9c(-c9ccc%10c(c9)c9ccccc9n%10-c9ccccc9)c8n(c5nc4c(c1)c23)c67 ; CC(C)(C)c1cc2c3cc4ccccc4c(N(c4ccccc4)c4ccccc4)c3n3c4cc5c6cc(C(C)(C)C)cc7c8cc9ccccc9c(N(c9ccccc9)c9ccccc9)c8n(c5nc4c(c1)c23)c67]

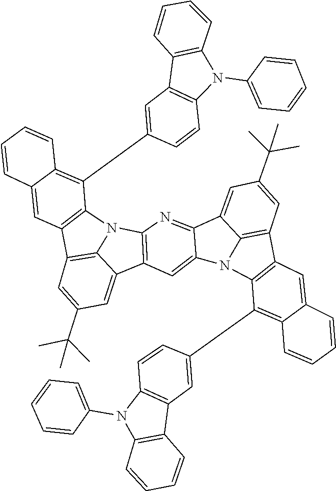 [SMILES: CC(C)(C)c1cc2c3cc4ccccc4c(-c4ccc5c(c4)c4ccccc4n5-c4ccccc4)c3n3c4cc5c6cc(C(C)(C)C)cc7c8cc9ccccc9c(-c9ccc%10c(c9)c9ccccc9n%10-c9ccccc9)c8n(c5nc4c(c1)c23)c67]

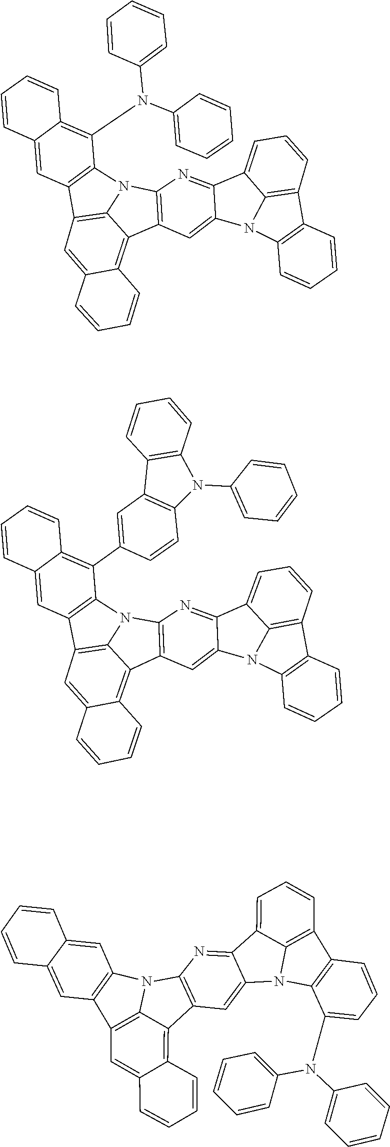 [SMILES: c1ccc(N(c2ccccc2)c2c3ccccc3cc3c4cc5ccccc5c5c6cc7c(nc6n(c23)c45)c2cccc3c4ccccc4n7c32)cc1 ; c1ccc(-n2c3ccccc3c3cc(-c4c5ccccc5cc5c6cc7ccccc7c7c8cc9c(nc8n(c45)c67)c4cccc5c6ccccc6n9c54)ccc32)cc1 ; c1ccc(N(c2ccccc2)c2cccc3c4cccc5c6nc7c(cc6n(c23)c45)c2c3ccccc3cc3c4cc5ccccc5cc4n7c32)cc1]

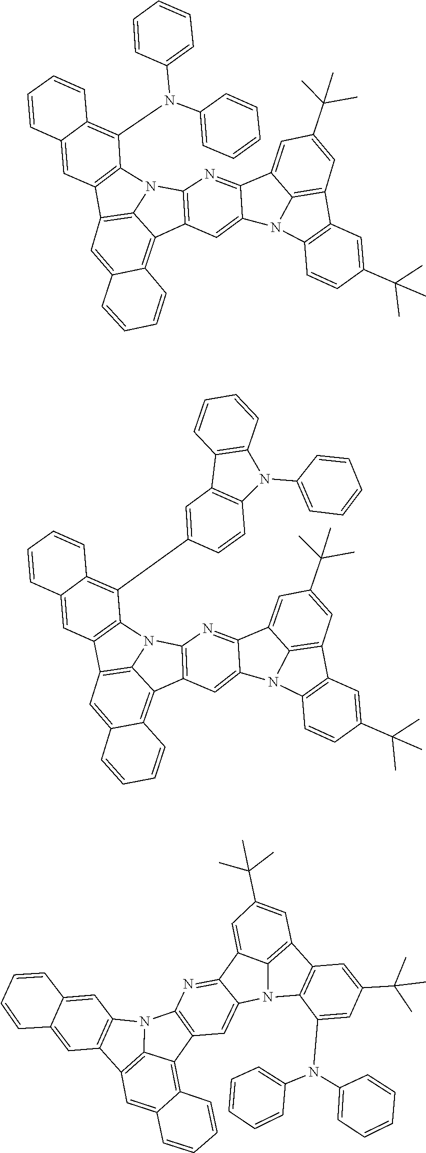 [SMILES: CC(C)(C)c1ccc2c(c1)c1cc(C(C)(C)C)cc3c4nc5c(cc4n2c13)c1c2ccccc2cc2c3cc4ccccc4c(N(c4ccccc4)c4ccccc4)c3n5c21 ; CC(C)(C)c1ccc2c(c1)c1cc(C(C)(C)C)cc3c4nc5c(cc4n2c13)c1c2ccccc2cc2c3cc4ccccc4c(-c4ccc6c(c4)c4ccccc4n6-c4ccccc4)c3n5c21 ; CC(C)(C)c1cc(N(c2ccccc2)c2ccccc2)c2c(c1)c1cc(C(C)(C)C)cc3c4nc5c(cc4n2c13)c1c2ccccc2cc2c3cc4ccccc4cc3n5c21]

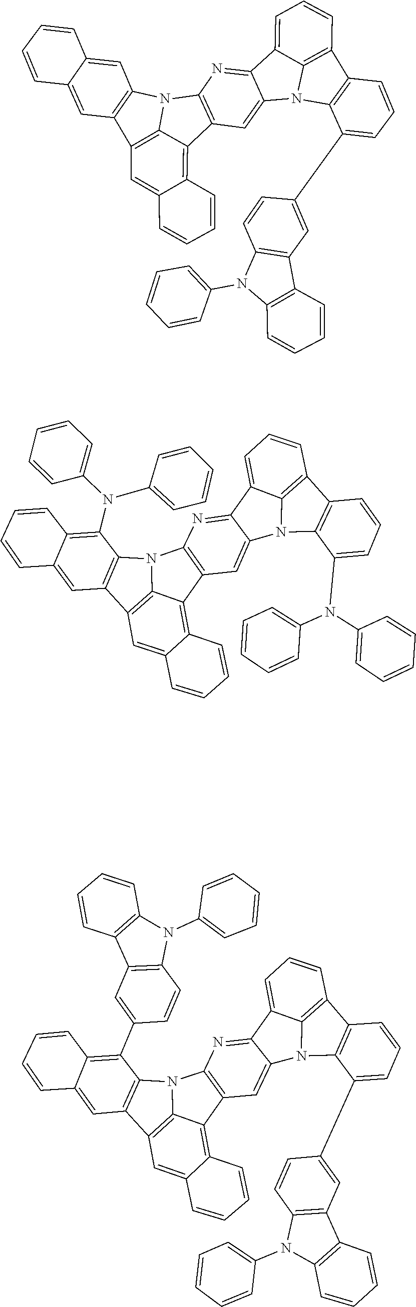 [SMILES: c1ccc(-n2c3ccccc3c3cc(-c4cccc5c6cccc7c8nc9c(cc8n(c45)c67)c4c5ccccc5cc5c6cc7ccccc7cc6n9c54)ccc32)cc1 ; c1ccc(N(c2ccccc2)c2cccc3c4cccc5c6nc7c(cc6n(c23)c45)c2c3ccccc3cc3c4cc5ccccc5c(N(c5ccccc5)c5ccccc5)c4n7c32)cc1 ; c1ccc(-n2c3ccccc3c3cc(-c4cccc5c6cccc7c8nc9c(cc8n(c45)c67)c4c5ccccc5cc5c6cc7ccccc7c(-c7ccc8c(c7)c7ccccc7n8-c7ccccc7)c6n9c54)ccc32)cc1]

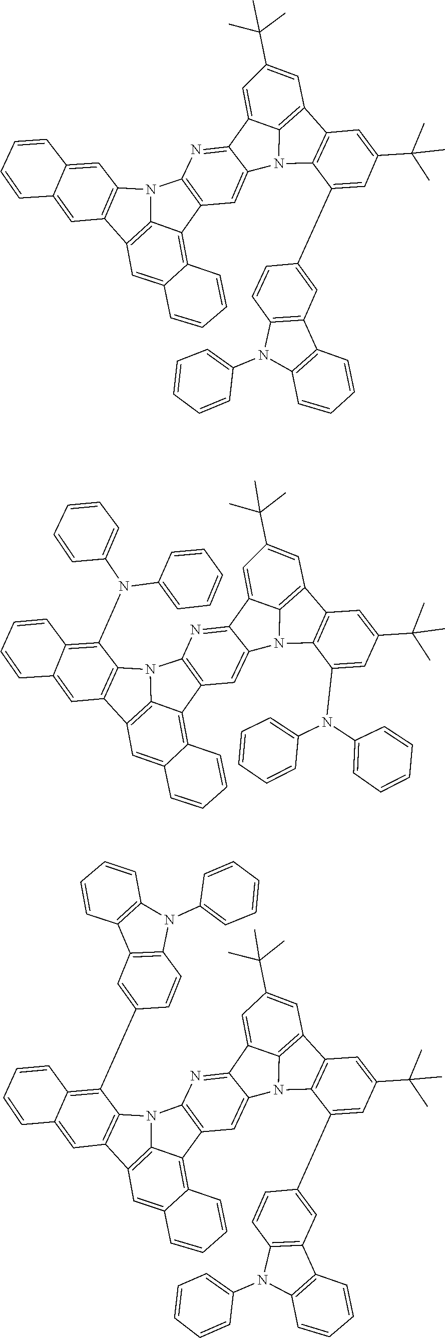 [SMILES: CC(C)(C)c1cc(-c2ccc3c(c2)c2ccccc2n3-c2ccccc2)c2c(c1)c1cc(C(C)(C)C)cc3c4nc5c(cc4n2c13)c1c2ccccc2cc2c3cc4ccccc4cc3n5c21 ; CC(C)(C)c1cc(N(c2ccccc2)c2ccccc2)c2c(c1)c1cc(C(C)(C)C)cc3c4nc5c(cc4n2c13)c1c2ccccc2cc2c3cc4ccccc4c(N(c4ccccc4)c4ccccc4)c3n5c21 ; CC(C)(C)c1cc(-c2ccc3c(c2)c2ccccc2n3-c2ccccc2)c2c(c1)c1cc(C(C)(C)C)cc3c4nc5c(cc4n2c13)c1c2ccccc2cc2c3cc4ccccc4c(-c4ccc6c(c4)c4ccccc4n6-c4ccccc4)c3n5c21]

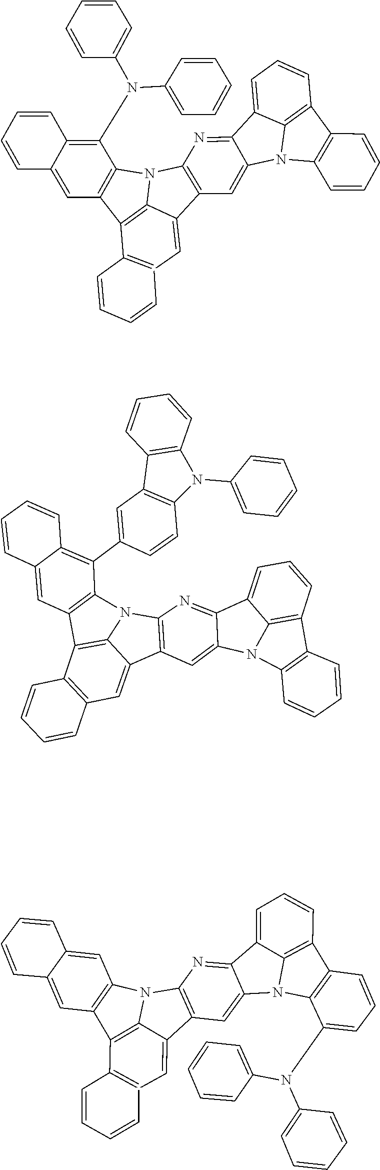 [SMILES: C/C=C\C=C/c1ccc2c3cc4c(nc3n3c5c(N(c6ccccc6)c6ccccc6)c6ccccc6cc5c1c23)c1cccc2c3ccccc3n4c21 ; c1ccc(-n2c3ccccc3c3cc(-c4c5ccccc5cc5c6c7ccccc7cc7c8cc9c(nc8n(c45)c76)c4cccc5c6ccccc6n9c54)ccc32)cc1 ; C/C=C\C=C/c1ccc2c3cc4c(nc3n3c5cc6ccccc6cc5c1c23)c1cccc2c3cccc(N(c5ccccc5)c5ccccc5)c3n4c21]

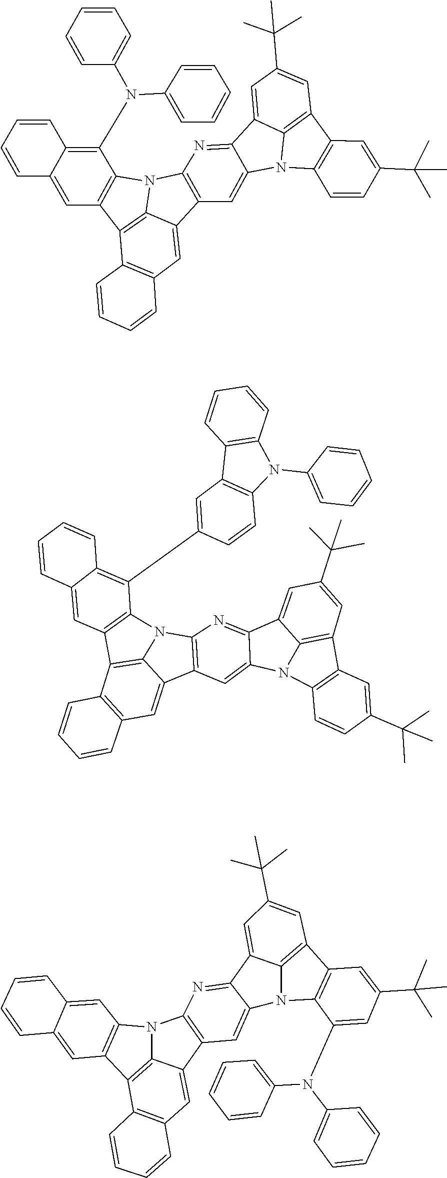 [SMILES: CC(C)(C)c1ccc2c(c1)c1cc(C(C)(C)C)cc3c4nc5c(cc4n2c13)c1cc2ccccc2c2c3cc4ccccc4c(N(c4ccccc4)c4ccccc4)c3n5c12 ; CC(C)(C)c1ccc2c(c1)c1cc(C(C)(C)C)cc3c4nc5c(cc4n2c13)c1cc2ccccc2c2c3cc4ccccc4c(-c4ccc6c(c4)c4ccccc4n6-c4ccccc4)c3n5c12 ; CC(C)(C)c1cc(N(c2ccccc2)c2ccccc2)c2c(c1)c1cc(C(C)(C)C)cc3c4nc5c(cc4n2c13)c1cc2ccccc2c2c3cc4ccccc4cc3n5c12]

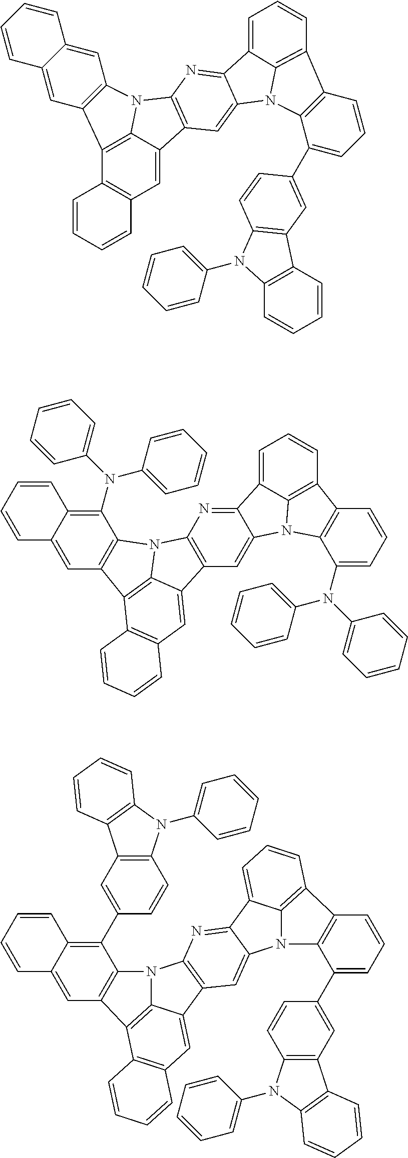 [SMILES: c1ccc(-n2c3ccccc3c3cc(-c4cccc5c6cccc7c8nc9c(cc8n(c45)c67)c4cc5ccccc5c5c6cc7ccccc7cc6n9c45)ccc32)cc1 ; c1ccc(N(c2ccccc2)c2cccc3c4cccc5c6nc7c(cc6n(c23)c45)c2cc3ccccc3c3c4cc5ccccc5c(N(c5ccccc5)c5ccccc5)c4n7c23)cc1 ; c1ccc(-n2c3ccccc3c3cc(-c4cccc5c6cccc7c8nc9c(cc8n(c45)c67)c4cc5ccccc5c5c6cc7ccccc7c(-c7ccc8c(c7)c7ccccc7n8-c7ccccc7)c6n9c45)ccc32)cc1]

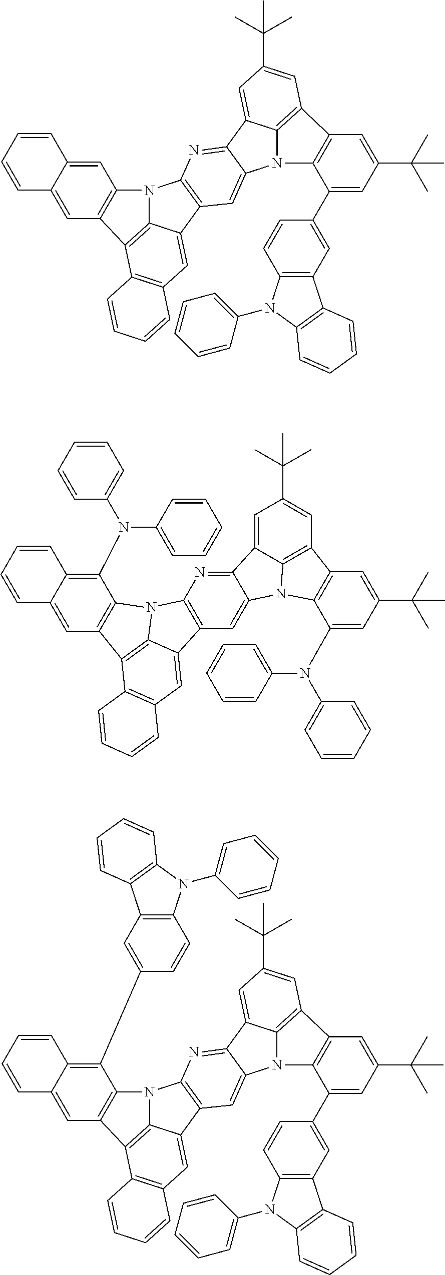 [SMILES: CC(C)(C)c1cc(-c2ccc3c(c2)c2ccccc2n3-c2ccccc2)c2c(c1)c1cc(C(C)(C)C)cc3c4nc5c(cc4n2c13)c1cc2ccccc2c2c3cc4ccccc4cc3n5c12 ; CC(C)(C)c1cc(N(c2ccccc2)c2ccccc2)c2c(c1)c1cc(C(C)(C)C)cc3c4nc5c(cc4n2c13)c1cc2ccccc2c2c3cc4ccccc4c(N(c4ccccc4)c4ccccc4)c3n5c12 ; CC(C)(C)c1cc(-c2ccc3c(c2)c2ccccc2n3-c2ccccc2)c2c(c1)c1cc(C(C)(C)C)cc3c4nc5c(cc4n2c13)c1cc2ccccc2c2c3cc4ccccc4c(-c4ccc6c(c4)c4ccccc4n6-c4ccccc4)c3n5c12]

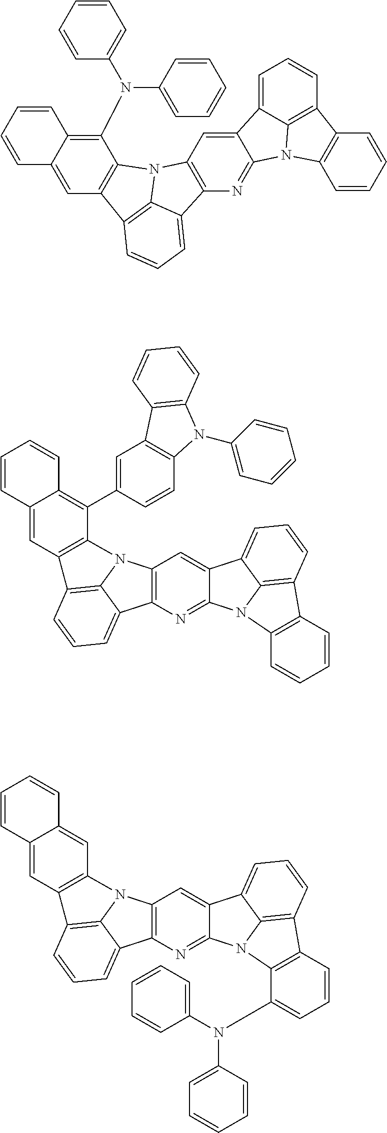 [SMILES: c1ccc(N(c2ccccc2)c2c3ccccc3cc3c4cccc5c6nc7c(cc6n(c23)c45)c2cccc3c4ccccc4n7c32)cc1 ; c1ccc(-n2c3ccccc3c3cc(-c4c5ccccc5cc5c6cccc7c8nc9c(cc8n(c45)c67)c4cccc5c6ccccc6n9c54)ccc32)cc1 ; c1ccc(N(c2ccccc2)c2cccc3c4cccc5c6cc7c(nc6n(c23)c54)c2cccc3c4cc5ccccc5cc4n7c32)cc1]

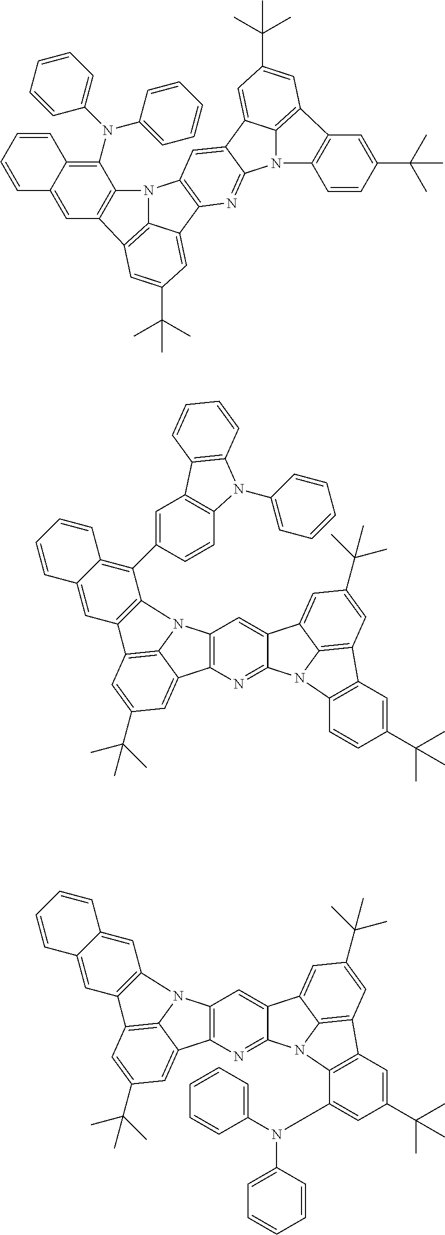 [SMILES: CC(C)(C)c1ccc2c(c1)c1cc(C(C)(C)C)cc3c4cc5c(nc4n2c13)c1cc(C(C)(C)C)cc2c3cc4ccccc4c(N(c4ccccc4)c4ccccc4)c3n5c21 ; CC(C)(C)c1ccc2c(c1)c1cc(C(C)(C)C)cc3c4cc5c(nc4n2c13)c1cc(C(C)(C)C)cc2c3cc4ccccc4c(-c4ccc6c(c4)c4ccccc4n6-c4ccccc4)c3n5c21 ; CC(C)(C)c1cc(N(c2ccccc2)c2ccccc2)c2c(c1)c1cc(C(C)(C)C)cc3c4cc5c(nc4n2c31)c1cc(C(C)(C)C)cc2c3cc4ccccc4cc3n5c21]

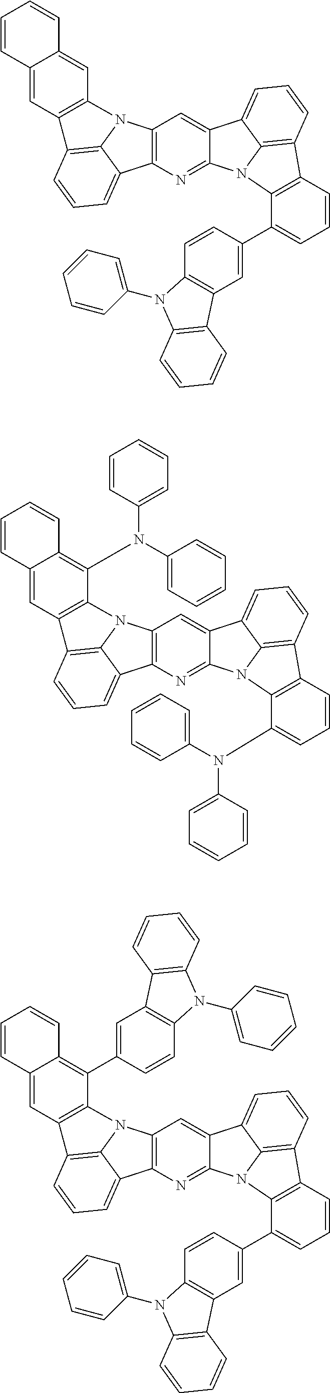 [SMILES: c1ccc(-n2c3ccccc3c3cc(-c4cccc5c6cccc7c8cc9c(nc8n(c45)c76)c4cccc5c6cc7ccccc7cc6n9c54)ccc32)cc1 ; c1ccc(N(c2ccccc2)c2c3ccccc3cc3c4cccc5c6nc7c(cc6n(c23)c45)c2cccc3c4cccc(N(c5ccccc5)c5ccccc5)c4n7c23)cc1 ; c1ccc(-n2c3ccccc3c3cc(-c4c5ccccc5cc5c6cccc7c8nc9c(cc8n(c45)c67)c4cccc5c6cccc(-c7ccc8c(c7)c7ccccc7n8-c7ccccc7)c6n9c45)ccc32)cc1]

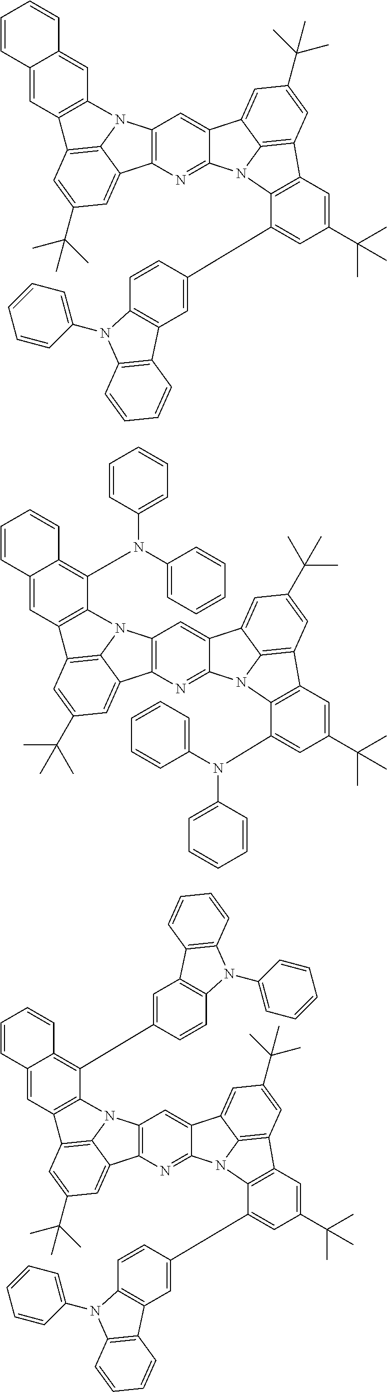

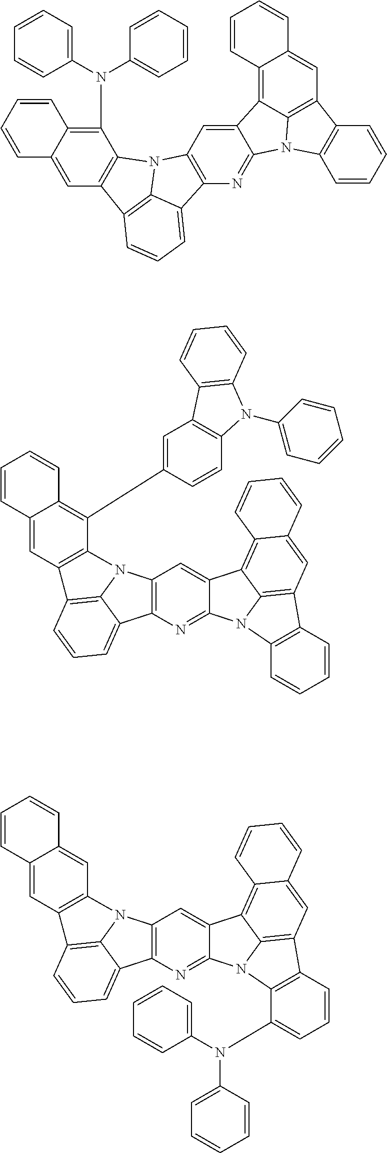 [SMILES: c1ccc(N(c2ccccc2)c2c3ccccc3cc3c4cccc5c6nc7c(cc6n(c23)c45)c2c3ccccc3cc3c4ccccc4n7c32)cc1 ; c1ccc(-n2c3ccccc3c3cc(-c4c5ccccc5cc5c6cccc7c8nc9c(cc8n(c45)c67)c4c5ccccc5cc5c6ccccc6n9c54)ccc32)cc1 ; c1ccc(N(c2ccccc2)c2cccc3c4cc5ccccc5c5c6cc7c(nc6n(c23)c45)c2cccc3c4cc5ccccc5cc4n7c32)cc1]

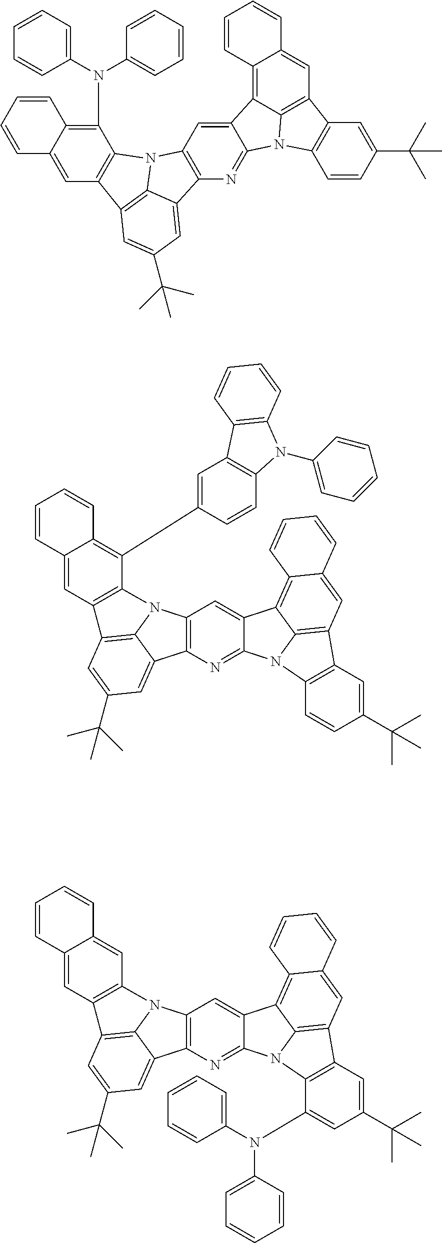 [SMILES: CC(C)(C)c1ccc2c(c1)c1cc3ccccc3c3c4cc5c(nc4n2c13)c1cc(C(C)(C)C)cc2c3cc4ccccc4c(N(c4ccccc4)c4ccccc4)c3n5c21 ; CC(C)(C)c1ccc2c(c1)c1cc3ccccc3c3c4cc5c(nc4n2c13)c1cc(C(C)(C)C)cc2c3cc4ccccc4c(-c4ccc6c(c4)c4ccccc4n6-c4ccccc4)c3n5c21 ; CC(C)(C)c1cc(N(c2ccccc2)c2ccccc2)c2c(c1)c1cc3ccccc3c3c4cc5c(nc4n2c13)c1cc(C(C)(C)C)cc2c3cc4ccccc4cc3n5c21]

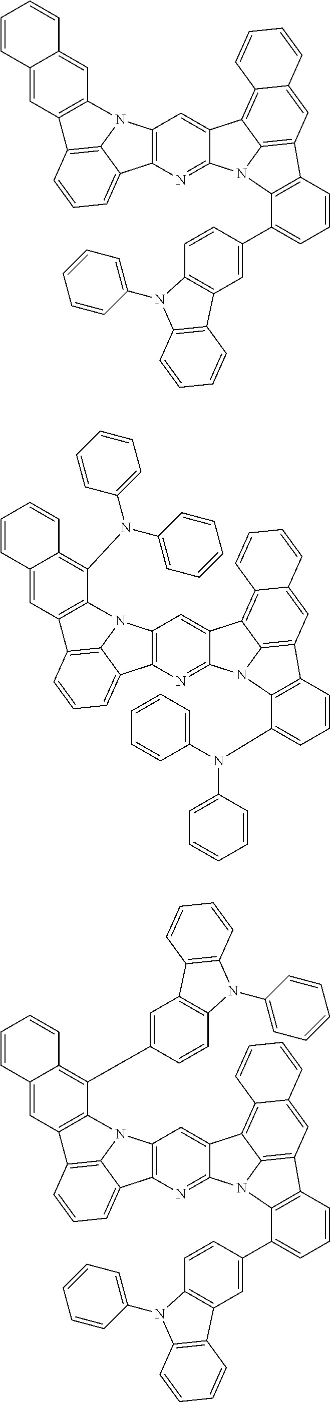

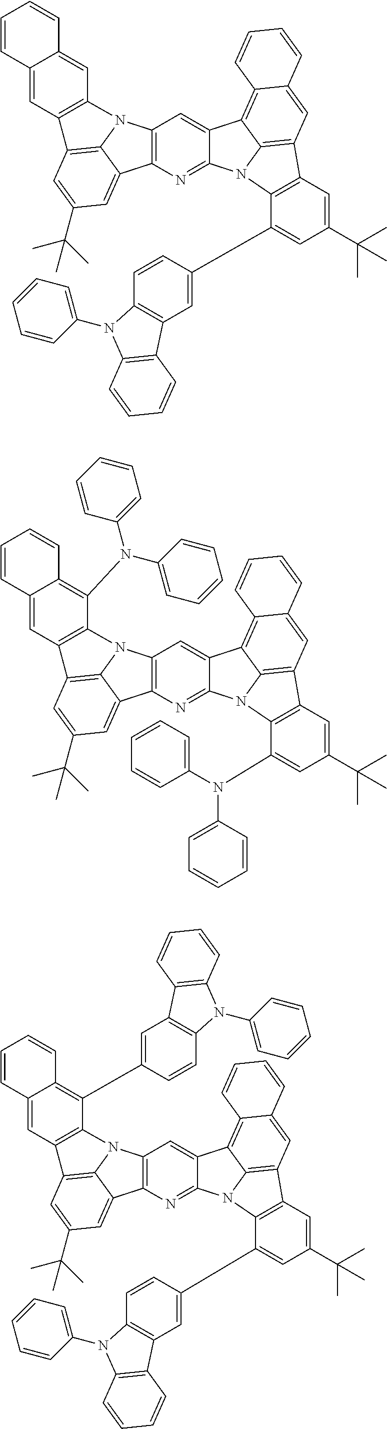

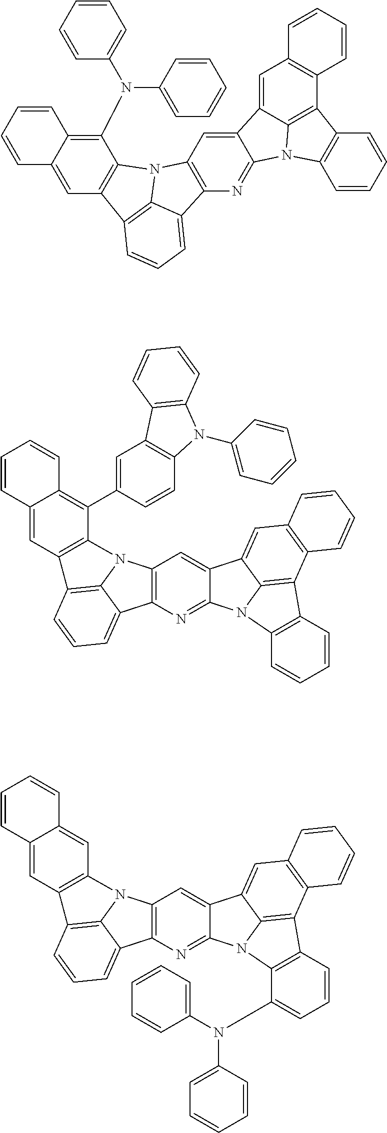 [SMILES: c1ccc(N(c2ccccc2)c2c3ccccc3cc3c4cccc5c6nc7c(cc6n(c23)c45)c2cc3ccccc3c3c4ccccc4n7c23)cc1 ; c1ccc(-n2c3ccccc3c3cc(-c4c5ccccc5cc5c6cccc7c8nc9c(cc8n(c45)c67)c4cc5ccccc5c5c6ccccc6n9c45)ccc32)cc1 ; c1ccc(N(c2ccccc2)c2cccc3c4c5ccccc5cc5c6cc7c(nc6n(c23)c54)c2cccc3c4cc5ccccc5cc4n7c32)cc1]

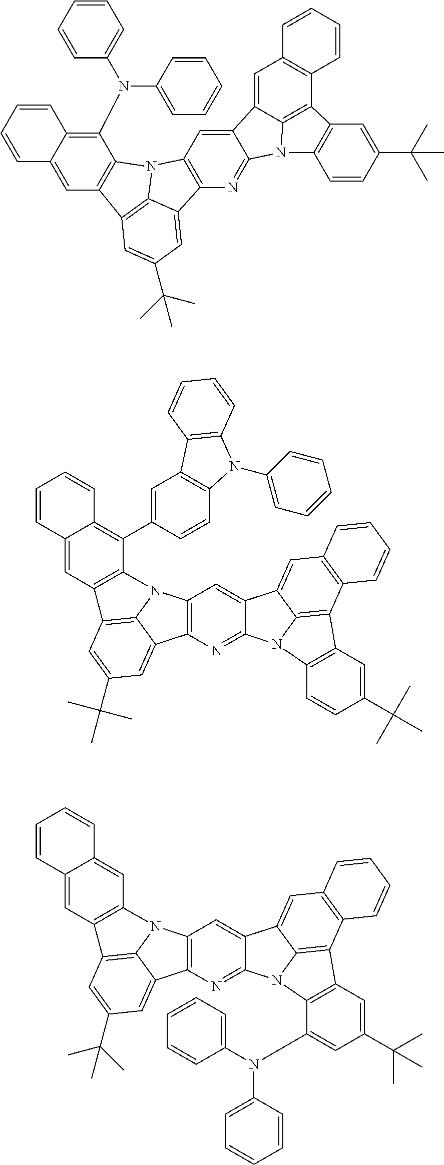 [SMILES: CC(C)(C)c1ccc2c(c1)c1c3ccccc3cc3c4cc5c(nc4n2c31)c1cc(C(C)(C)C)cc2c3cc4ccccc4c(N(c4ccccc4)c4ccccc4)c3n5c21 ; CC(C)(C)c1ccc2c(c1)c1c3ccccc3cc3c4cc5c(nc4n2c31)c1cc(C(C)(C)C)cc2c3cc4ccccc4c(-c4ccc6c(c4)c4ccccc4n6-c4ccccc4)c3n5c21 ; CC(C)(C)c1cc(N(c2ccccc2)c2ccccc2)c2c(c1)c1c3ccccc3cc3c4cc5c(nc4n2c31)c1cc(C(C)(C)C)cc2c3cc4ccccc4cc3n5c21]

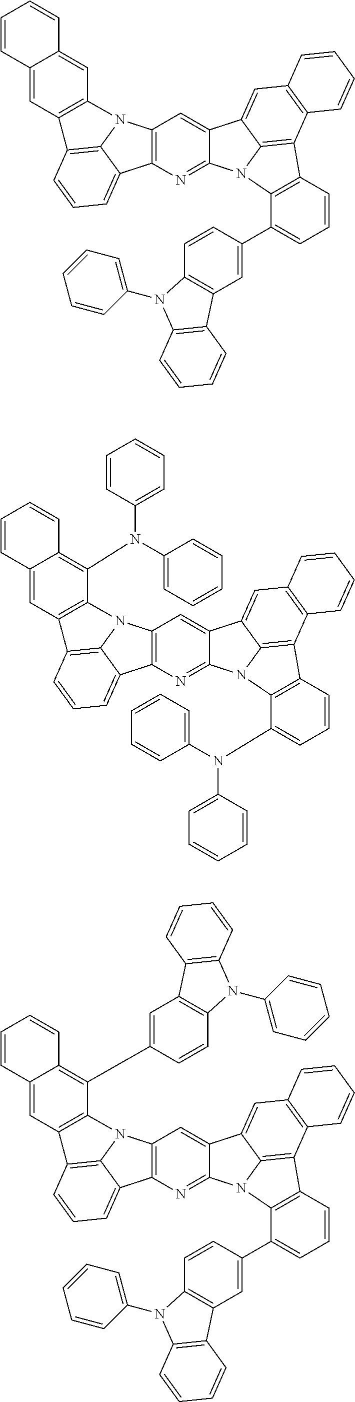

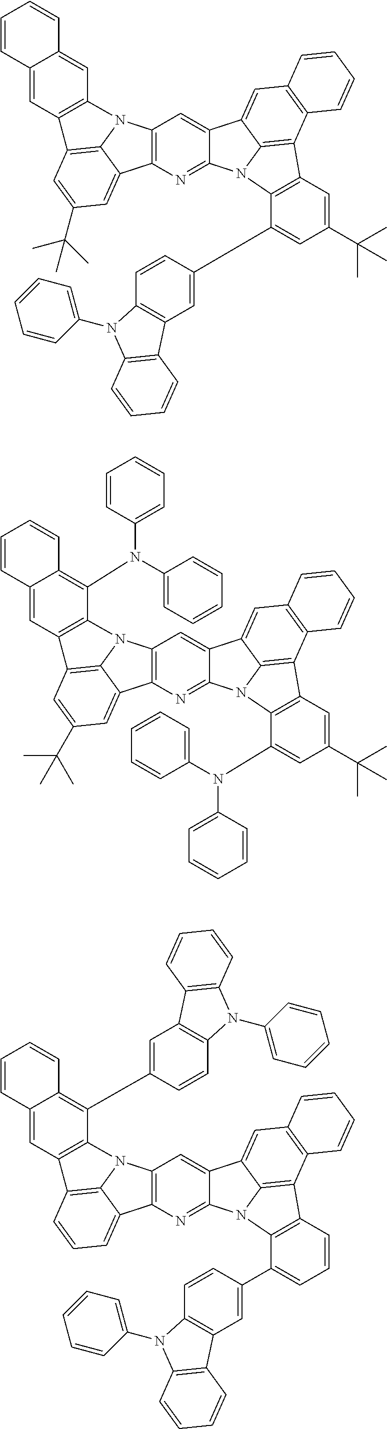

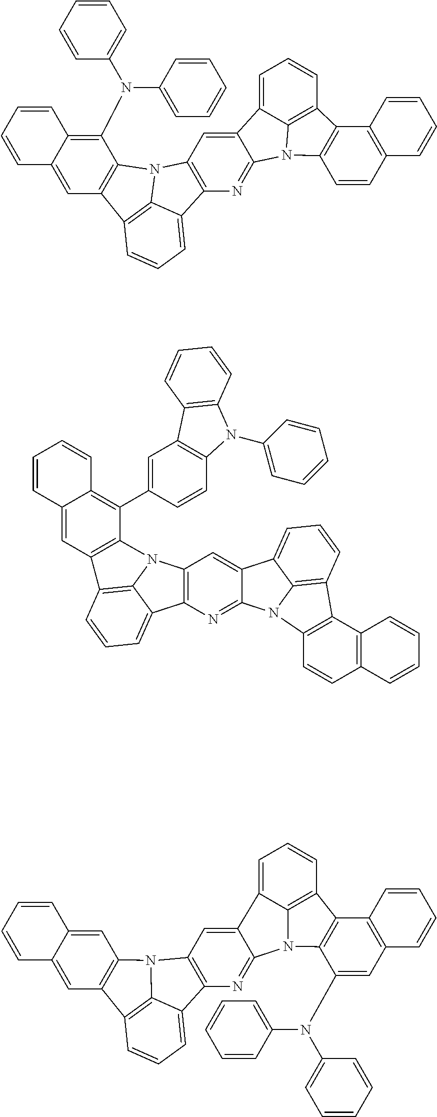 [SMILES: c1ccc(N(c2ccccc2)c2c3ccccc3cc3c4cccc5c6nc7c(cc6n(c23)c45)c2cccc3c4c5ccccc5ccc4n7c23)cc1 ; c1ccc(-n2c3ccccc3c3cc(-c4c5ccccc5cc5c6cccc7c8nc9c(cc8n(c45)c67)c4cccc5c6c7ccccc7ccc6n9c45)ccc32)cc1 ; c1ccc(N(c2ccccc2)c2cc3ccccc3c3c4cccc5c6cc7c(nc6n(c23)c54)c2cccc3c4cc5ccccc5cc4n7c32)cc1]

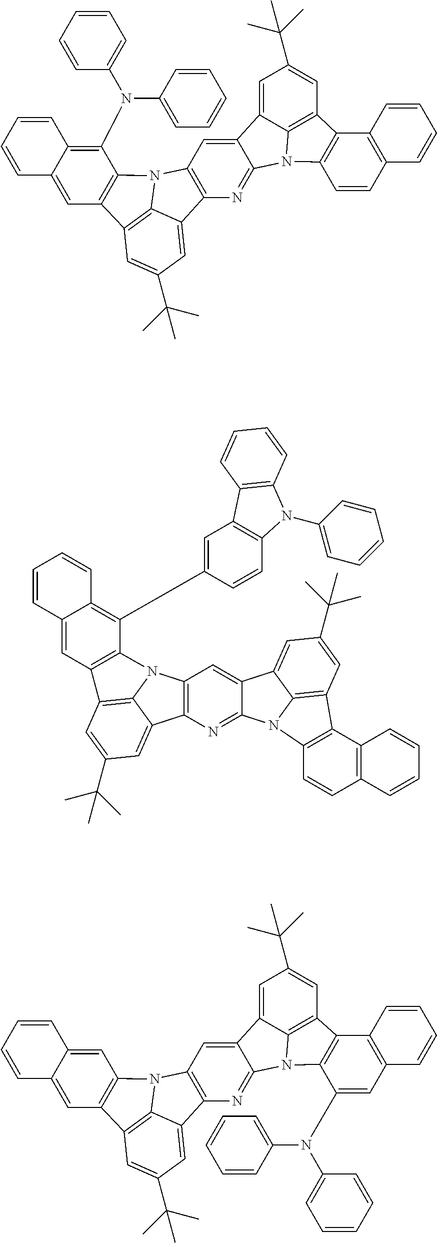 [SMILES: CC(C)(C)c1cc2c3cc4c(nc3n3c5ccc6ccccc6c5c(c1)c23)c1cc(C(C)(C)C)cc2c3cc5ccccc5c(N(c5ccccc5)c5ccccc5)c3n4c21 ; CC(C)(C)c1cc2c3cc4c(nc3n3c5ccc6ccccc6c5c(c1)c23)c1cc(C(C)(C)C)cc2c3cc5ccccc5c(-c5ccc6c(c5)c5ccccc5n6-c5ccccc5)c3n4c21 ; CC(C)(C)c1cc2c3cc4ccccc4cc3n3c4cc5c6cc(C(C)(C)C)cc7c8c9ccccc9cc(N(c9ccccc9)c9ccccc9)c8n(c5nc4c(c1)c23)c67]

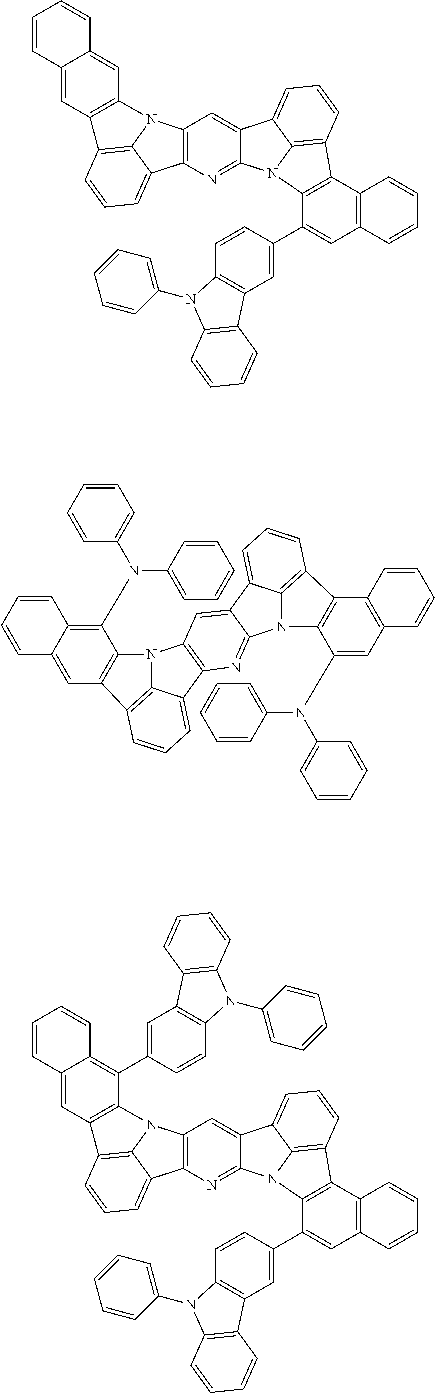 [SMILES: c1ccc(-n2c3ccccc3c3cc(-c4cc5ccccc5c5c6cccc7c8cc9c(nc8n(c45)c76)c4cccc5c6cc7ccccc7cc6n9c54)ccc32)cc1 ; c1ccc(N(c2ccccc2)c2c3ccccc3cc3c4cccc5c6nc7c(cc6n(c23)c45)c2cccc3c4c5ccccc5cc(N(c5ccccc5)c5ccccc5)c4n7c23)cc1 ; c1ccc(-n2c3ccccc3c3cc(-c4c5ccccc5cc5c6cccc7c8nc9c(cc8n(c45)c67)c4cccc5c6c7ccccc7cc(-c7ccc8c(c7)c7ccccc7n8-c7ccccc7)c6n9c45)ccc32)cc1]

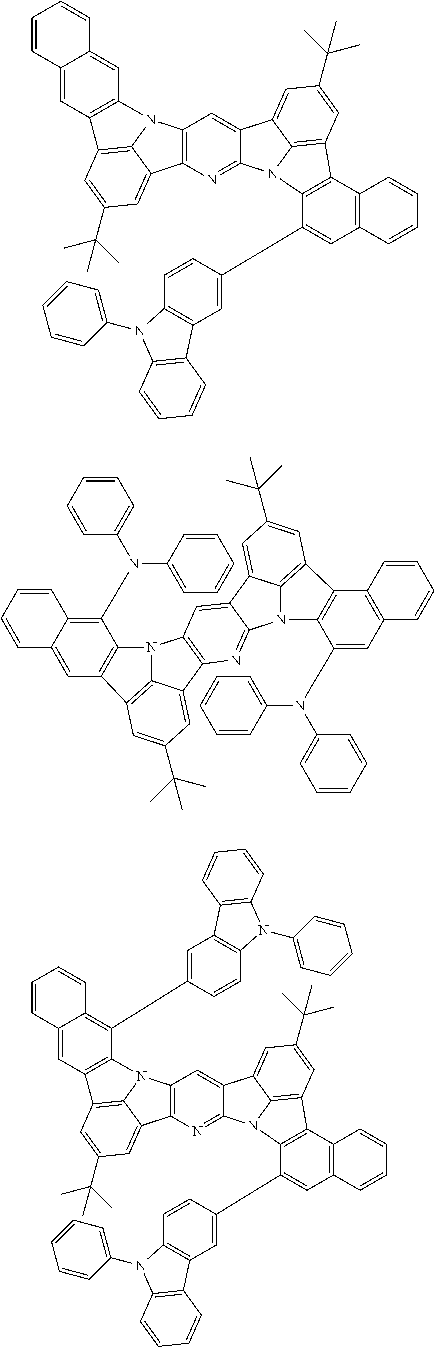 [SMILES: CC(C)(C)c1cc2c3cc4ccccc4cc3n3c4cc5c6cc(C(C)(C)C)cc7c8c9ccccc9cc(-c9ccc%10c(c9)c9ccccc9n%10-c9ccccc9)c8n(c5nc4c(c1)c23)c67 ; CC(C)(C)c1cc2c3cc4ccccc4c(N(c4ccccc4)c4ccccc4)c3n3c4cc5c6cc(C(C)(C)C)cc7c8c9ccccc9cc(N(c9ccccc9)c9ccccc9)c8n(c5nc4c(c1)c23)c67 ; CC(C)(C)c1cc2c3cc4ccccc4c(-c4ccc5c(c4)c4ccccc4n5-c4ccccc4)c3n3c4cc5c6cc(C(C)(C)C)cc7c8c9ccccc9cc(-c9ccc%10c(c9)c9ccccc9n%10-c9ccccc9)c8n(c5nc4c(c1)c23)c67]

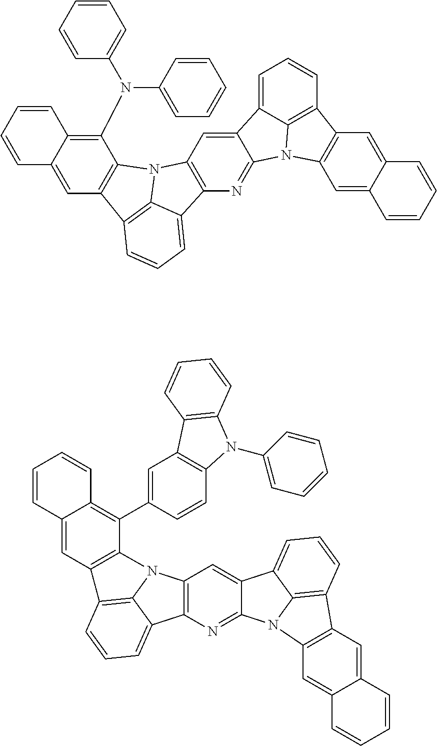 [SMILES: c1ccc(N(c2ccccc2)c2c3ccccc3cc3c4cccc5c6nc7c(cc6n(c23)c45)c2cccc3c4cc5ccccc5cc4n7c32)cc1 ; c1ccc(-n2c3ccccc3c3cc(-c4c5ccccc5cc5c6cccc7c8nc9c(cc8n(c45)c67)c4cccc5c6cc7ccccc7cc6n9c54)ccc32)cc1]

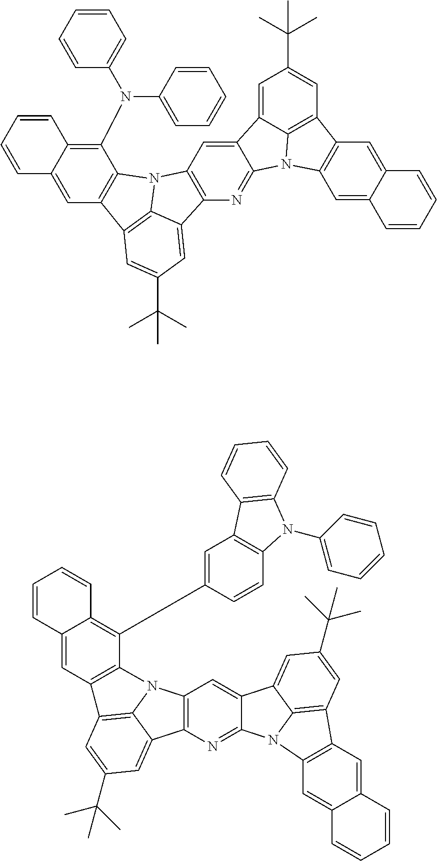 [SMILES: CC(C)(C)c1cc2c3cc4ccccc4cc3n3c4nc5c6cc(C(C)(C)C)cc7c8cc9ccccc9c(N(c9ccccc9)c9ccccc9)c8n(c5cc4c(c1)c23)c76 ; CC(C)(C)c1cc2c3cc4ccccc4cc3n3c4nc5c6cc(C(C)(C)C)cc7c8cc9ccccc9c(-c9ccc%10c(c9)c9ccccc9n%10-c9ccccc9)c8n(c5cc4c(c1)c23)c76]

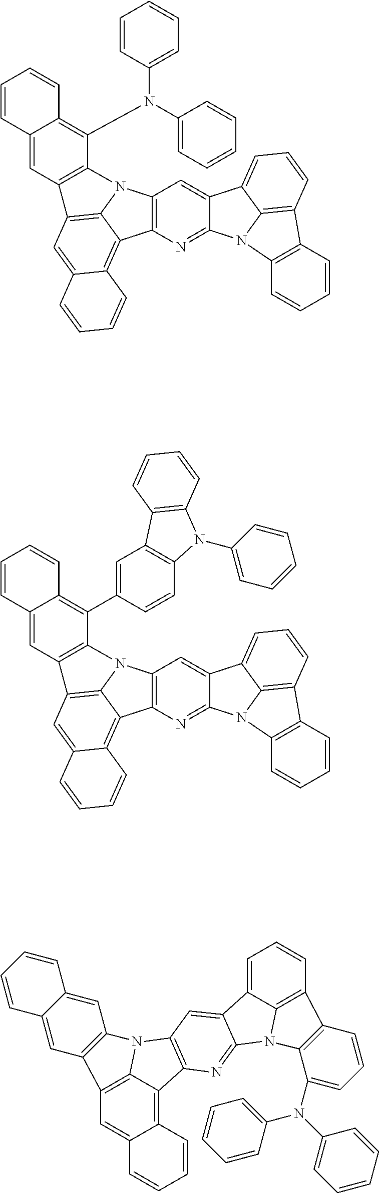 [SMILES: c1ccc(N(c2ccccc2)c2c3ccccc3cc3c4cc5ccccc5c5c6nc7c(cc6n(c23)c45)c2cccc3c4ccccc4n7c32)cc1 ; c1ccc(-n2c3ccccc3c3cc(-c4c5ccccc5cc5c6cc7ccccc7c7c8nc9c(cc8n(c45)c67)c4cccc5c6ccccc6n9c54)ccc32)cc1 ; c1ccc(N(c2ccccc2)c2cccc3c4cccc5c6cc7c(nc6n(c23)c54)c2c3ccccc3cc3c4cc5ccccc5cc4n7c32)cc1]

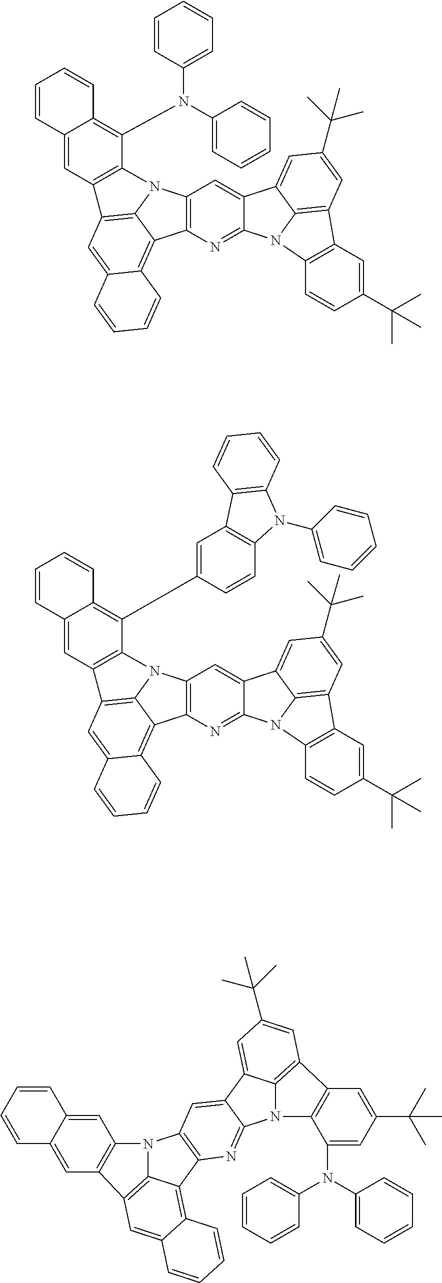 [SMILES: CC(C)(C)c1ccc2c(c1)c1cc(C(C)(C)C)cc3c4cc5c(nc4n2c13)c1c2ccccc2cc2c3cc4ccccc4c(N(c4ccccc4)c4ccccc4)c3n5c21 ; CC(C)(C)c1ccc2c(c1)c1cc(C(C)(C)C)cc3c4cc5c(nc4n2c13)c1c2ccccc2cc2c3cc4ccccc4c(-c4ccc6c(c4)c4ccccc4n6-c4ccccc4)c3n5c21 ; CC(C)(C)c1cc(N(c2ccccc2)c2ccccc2)c2c(c1)c1cc(C(C)(C)C)cc3c4cc5c(nc4n2c31)c1c2ccccc2cc2c3cc4ccccc4cc3n5c21]

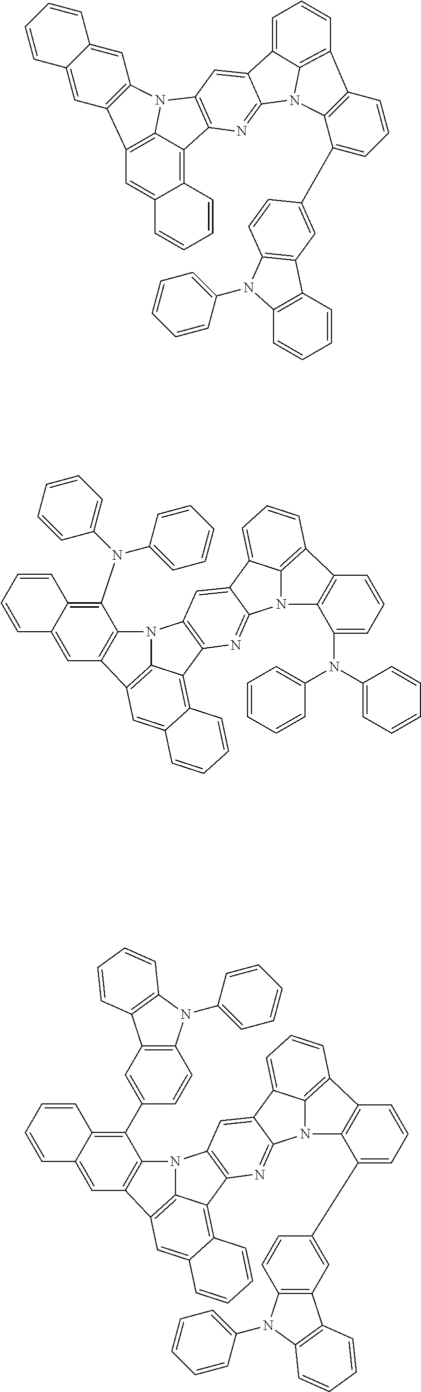 [SMILES: c1ccc(-n2c3ccccc3c3cc(-c4cccc5c6cccc7c8cc9c(nc8n(c45)c76)c4c5ccccc5cc5c6cc7ccccc7cc6n9c54)ccc32)cc1 ; c1ccc(N(c2ccccc2)c2c3ccccc3cc3c4cc5ccccc5c5c6nc7c(cc6n(c23)c45)c2cccc3c4cccc(N(c5ccccc5)c5ccccc5)c4n7c23)cc1 ; c1ccc(-n2c3ccccc3c3cc(-c4c5ccccc5cc5c6cc7ccccc7c7c8nc9c(cc8n(c45)c67)c4cccc5c6cccc(-c7ccc8c(c7)c7ccccc7n8-c7ccccc7)c6n9c45)ccc32)cc1]

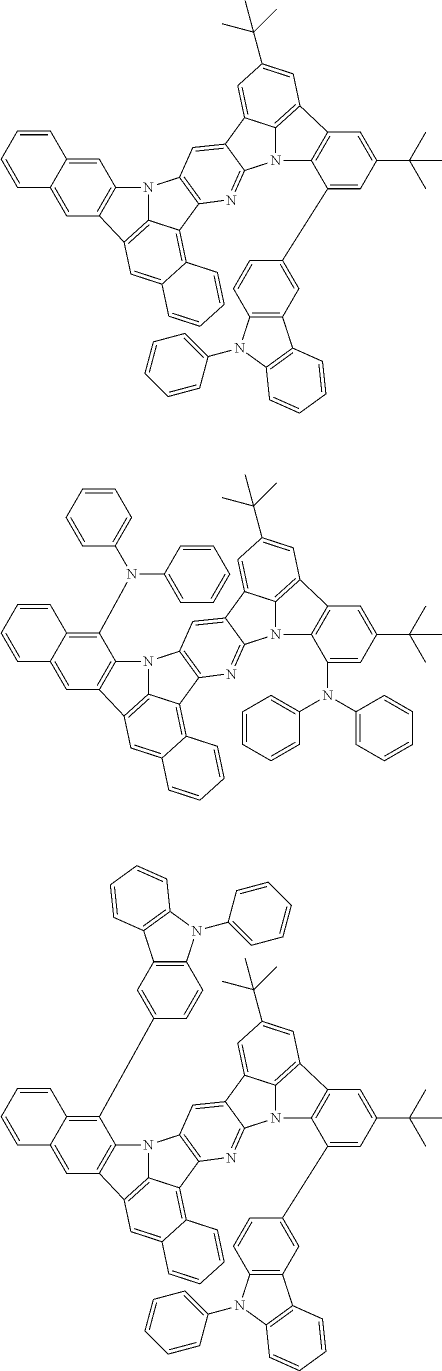 [SMILES: CC(C)(C)c1cc(-c2ccc3c(c2)c2ccccc2n3-c2ccccc2)c2c(c1)c1cc(C(C)(C)C)cc3c4cc5c(nc4n2c31)c1c2ccccc2cc2c3cc4ccccc4cc3n5c21 ; CC(C)(C)c1cc(N(c2ccccc2)c2ccccc2)c2c(c1)c1cc(C(C)(C)C)cc3c4cc5c(nc4n2c31)c1c2ccccc2cc2c3cc4ccccc4c(N(c4ccccc4)c4ccccc4)c3n5c21 ; CC(C)(C)c1cc(-c2ccc3c(c2)c2ccccc2n3-c2ccccc2)c2c(c1)c1cc(C(C)(C)C)cc3c4cc5c(nc4n2c31)c1c2ccccc2cc2c3cc4ccccc4c(-c4ccc6c(c4)c4ccccc4n6-c4ccccc4)c3n5c21]

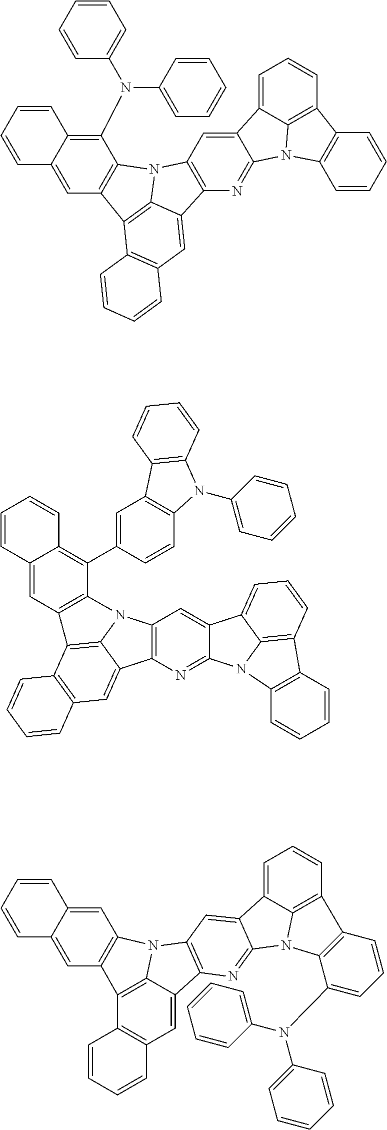 [SMILES: c1ccc(N(c2ccccc2)c2c3ccccc3cc3c4c5ccccc5cc5c6nc7c(cc6n(c23)c54)c2cccc3c4ccccc4n7c32)cc1 ; c1ccc(-n2c3ccccc3c3cc(-c4c5ccccc5cc5c6c7ccccc7cc7c8nc9c(cc8n(c45)c76)c4cccc5c6ccccc6n9c54)ccc32)cc1 ; c1ccc(N(c2ccccc2)c2cccc3c4cccc5c6cc7c(nc6n(c23)c54)c2cc3ccccc3c3c4cc5ccccc5cc4n7c23)cc1]

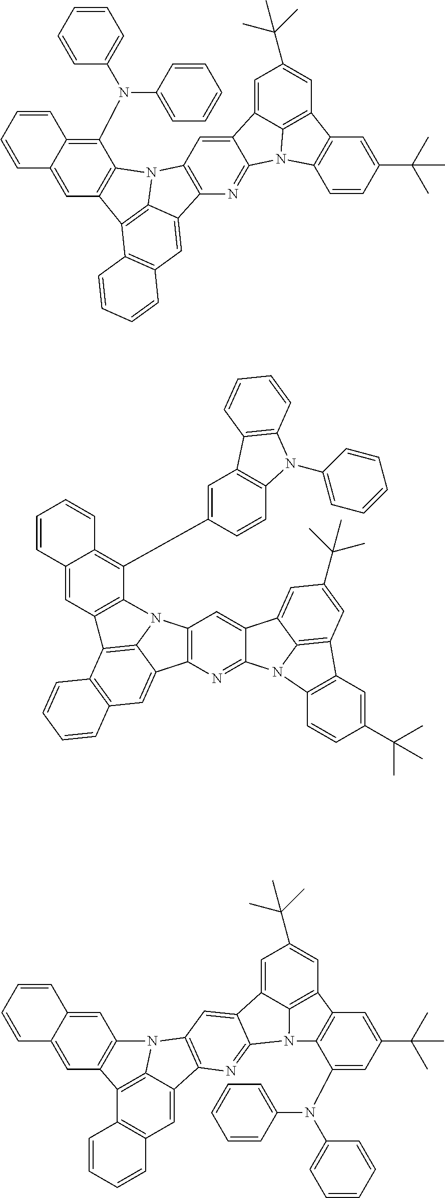 [SMILES: CC(C)(C)c1ccc2c(c1)c1cc(C(C)(C)C)cc3c4cc5c(nc4n2c13)c1cc2ccccc2c2c3cc4ccccc4c(N(c4ccccc4)c4ccccc4)c3n5c12 ; CC(C)(C)c1ccc2c(c1)c1cc(C(C)(C)C)cc3c4cc5c(nc4n2c13)c1cc2ccccc2c2c3cc4ccccc4c(-c4ccc6c(c4)c4ccccc4n6-c4ccccc4)c3n5c12 ; CC(C)(C)c1cc(N(c2ccccc2)c2ccccc2)c2c(c1)c1cc(C(C)(C)C)cc3c4cc5c(nc4n2c31)c1cc2ccccc2c2c3cc4ccccc4cc3n5c12]

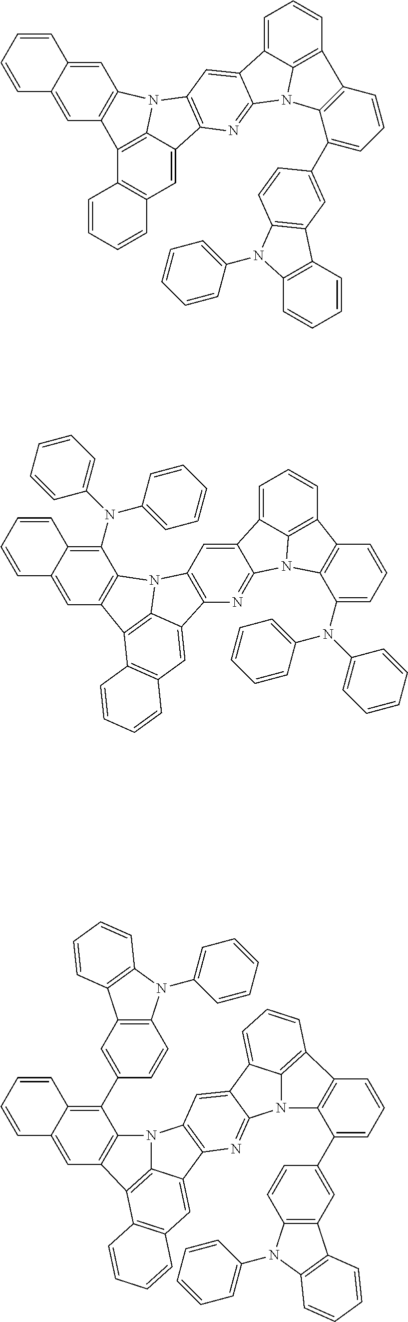 [SMILES: c1ccc(-n2c3ccccc3c3cc(-c4cccc5c6cccc7c8cc9c(nc8n(c45)c76)c4cc5ccccc5c5c6cc7ccccc7cc6n9c45)ccc32)cc1 ; c1ccc(N(c2ccccc2)c2c3ccccc3cc3c4c5ccccc5cc5c6nc7c(cc6n(c23)c54)c2cccc3c4cccc(N(c5ccccc5)c5ccccc5)c4n7c23)cc1 ; c1ccc(-n2c3ccccc3c3cc(-c4c5ccccc5cc5c6c7ccccc7cc7c8nc9c(cc8n(c45)c76)c4cccc5c6cccc(-c7ccc8c(c7)c7ccccc7n8-c7ccccc7)c6n9c45)ccc32)cc1]

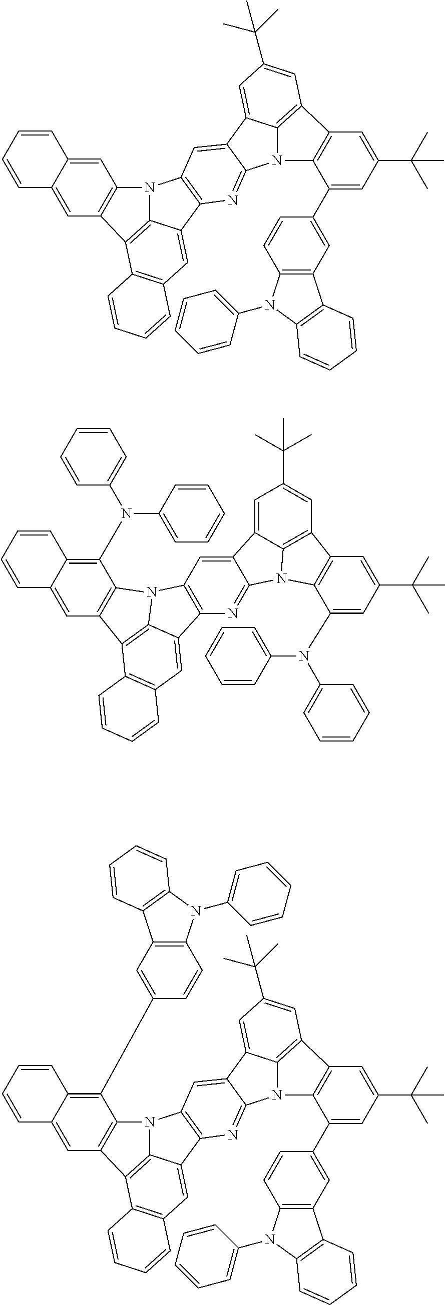 [SMILES: CC(C)(C)c1cc(-c2ccc3c(c2)c2ccccc2n3-c2ccccc2)c2c(c1)c1cc(C(C)(C)C)cc3c4cc5c(nc4n2c31)c1cc2ccccc2c2c3cc4ccccc4cc3n5c12 ; CC(C)(C)c1cc(N(c2ccccc2)c2ccccc2)c2c(c1)c1cc(C(C)(C)C)cc3c4cc5c(nc4n2c31)c1cc2ccccc2c2c3cc4ccccc4c(N(c4ccccc4)c4ccccc4)c3n5c12 ; CC(C)(C)c1cc(-c2ccc3c(c2)c2ccccc2n3-c2ccccc2)c2c(c1)c1cc(C(C)(C)C)cc3c4cc5c(nc4n2c31)c1cc2ccccc2c2c3cc4ccccc4c(-c4ccc6c(c4)c4ccccc4n6-c4ccccc4)c3n5c12]

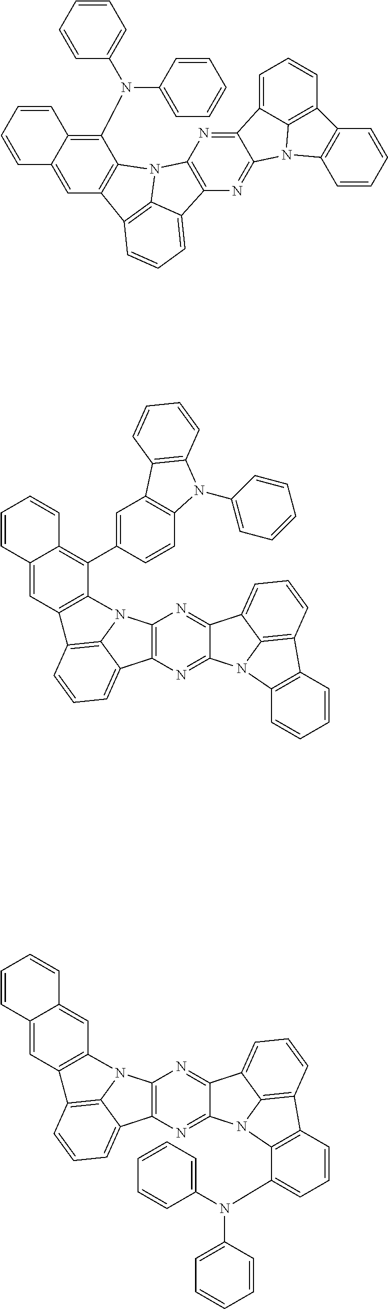 [SMILES: c1ccc(N(c2ccccc2)c2c3ccccc3cc3c4cccc5c6nc7c(nc6n(c23)c45)c2cccc3c4ccccc4n7c32)cc1 ; c1ccc(-n2c3ccccc3c3cc(-c4c5ccccc5cc5c6cccc7c8nc9c(nc8n(c45)c67)c4cccc5c6ccccc6n9c54)ccc32)cc1 ; c1ccc(N(c2ccccc2)c2cccc3c4cccc5c6nc7c(nc6n(c23)c45)c2cccc3c4cc5ccccc5cc4n7c32)cc1]

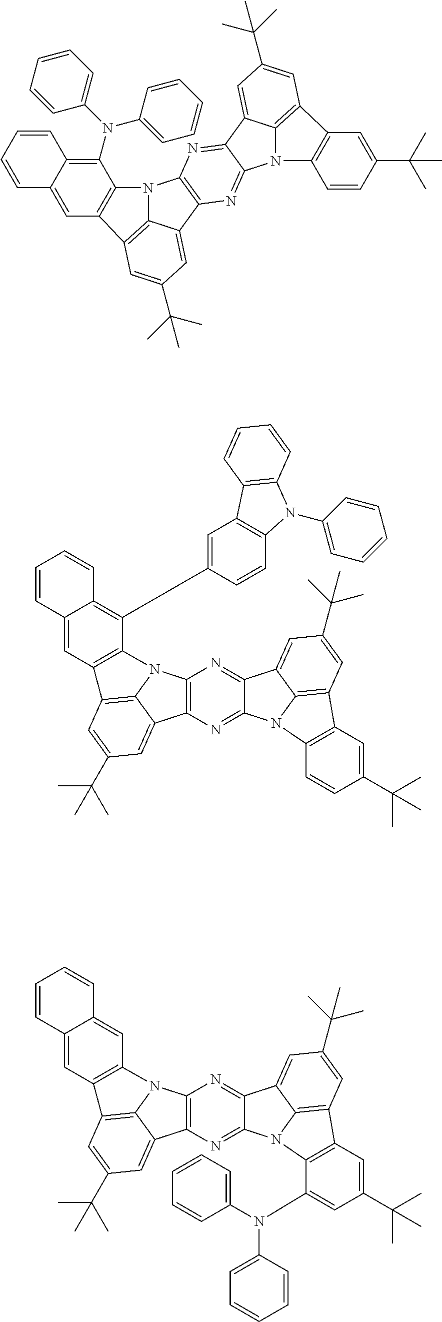 [SMILES: CC(C)(C)c1ccc2c(c1)c1cc(C(C)(C)C)cc3c4nc5c(nc4n2c13)c1cc(C(C)(C)C)cc2c3cc4ccccc4c(N(c4ccccc4)c4ccccc4)c3n5c21 ; CC(C)(C)c1ccc2c(c1)c1cc(C(C)(C)C)cc3c4nc5c(nc4n2c13)c1cc(C(C)(C)C)cc2c3cc4ccccc4c(-c4ccc6c(c4)c4ccccc4n6-c4ccccc4)c3n5c21 ; CC(C)(C)c1cc(N(c2ccccc2)c2ccccc2)c2c(c1)c1cc(C(C)(C)C)cc3c4nc5c(nc4n2c13)c1cc(C(C)(C)C)cc2c3cc4ccccc4cc3n5c21]

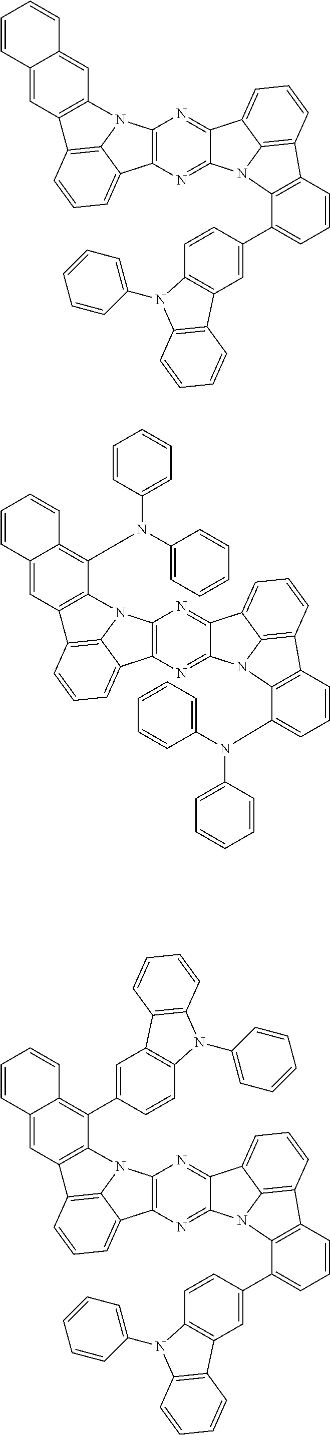 [SMILES: c1ccc(-n2c3ccccc3c3cc(-c4cccc5c6cccc7c8nc9c(nc8n(c45)c67)c4cccc5c6cc7ccccc7cc6n9c54)ccc32)cc1 ; c1ccc(N(c2ccccc2)c2cccc3c4cccc5c6nc7c(nc6n(c23)c45)c2cccc3c4cc5ccccc5c(N(c5ccccc5)c5ccccc5)c4n7c32)cc1 ; c1ccc(-n2c3ccccc3c3cc(-c4cccc5c6cccc7c8nc9c(nc8n(c45)c67)c4cccc5c6cc7ccccc7c(-c7ccc8c(c7)c7ccccc7n8-c7ccccc7)c6n9c54)ccc32)cc1]

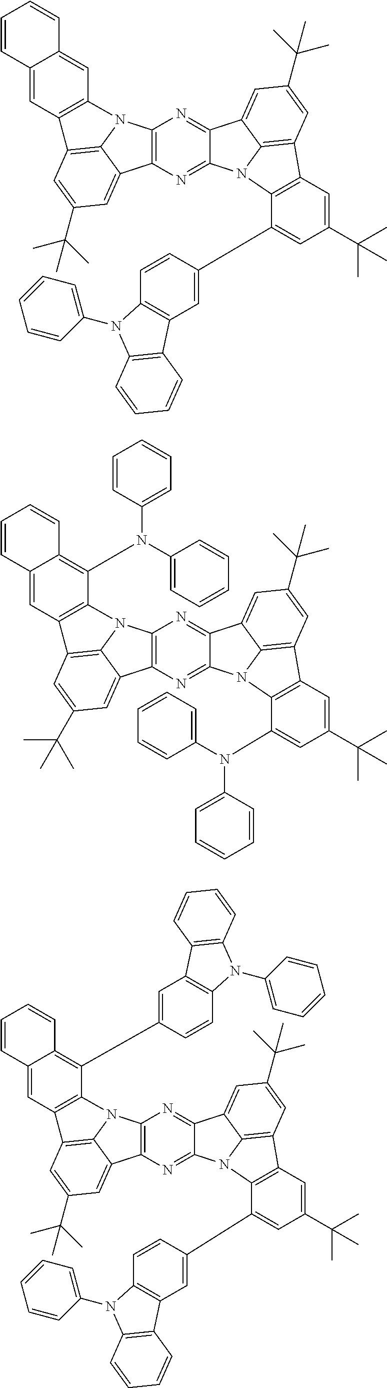

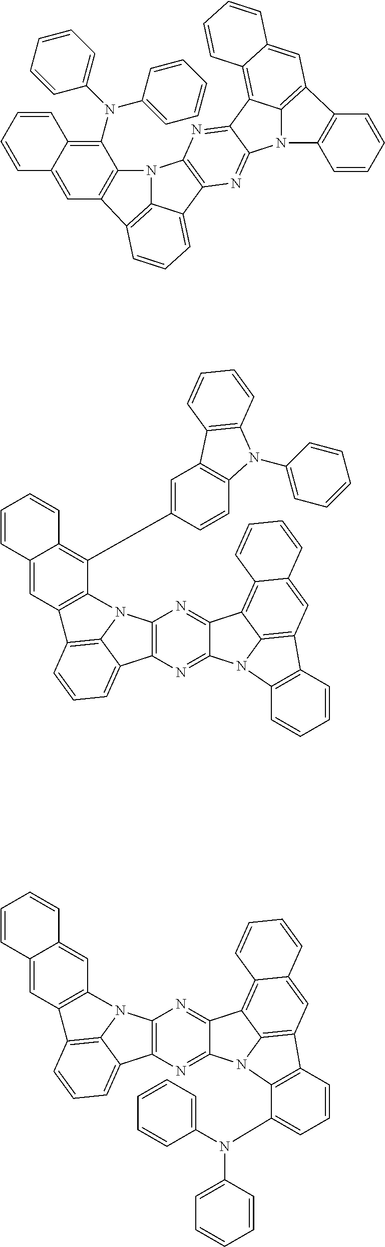 [SMILES: c1ccc(N(c2ccccc2)c2c3ccccc3cc3c4cccc5c6nc7c(nc6n(c23)c45)c2c3ccccc3cc3c4ccccc4n7c32)cc1 ; c1ccc(-n2c3ccccc3c3cc(-c4c5ccccc5cc5c6cccc7c8nc9c(nc8n(c45)c67)c4c5ccccc5cc5c6ccccc6n9c54)ccc32)cc1 ; c1ccc(N(c2ccccc2)c2cccc3c4cc5ccccc5c5c6nc7c(nc6n(c23)c45)c2cccc3c4cc5ccccc5cc4n7c32)cc1]

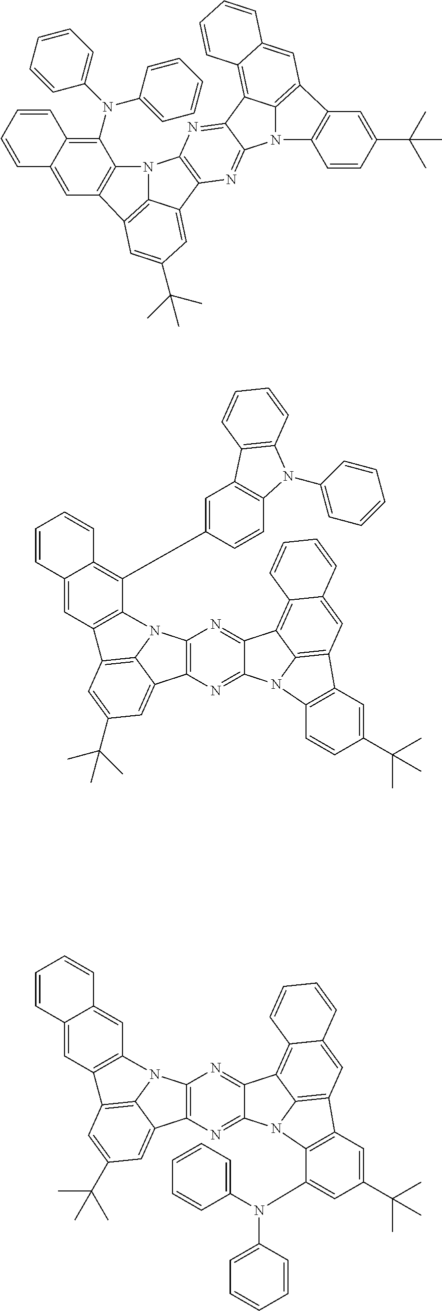 [SMILES: CC(C)(C)c1ccc2c(c1)c1cc3ccccc3c3c4nc5c(nc4n2c13)c1cc(C(C)(C)C)cc2c3cc4ccccc4c(N(c4ccccc4)c4ccccc4)c3n5c21 ; CC(C)(C)c1ccc2c(c1)c1cc3ccccc3c3c4nc5c(nc4n2c13)c1cc(C(C)(C)C)cc2c3cc4ccccc4c(-c4ccc6c(c4)c4ccccc4n6-c4ccccc4)c3n5c21 ; CC(C)(C)c1cc(N(c2ccccc2)c2ccccc2)c2c(c1)c1cc3ccccc3c3c4nc5c(nc4n2c13)c1cc(C(C)(C)C)cc2c3cc4ccccc4cc3n5c21]

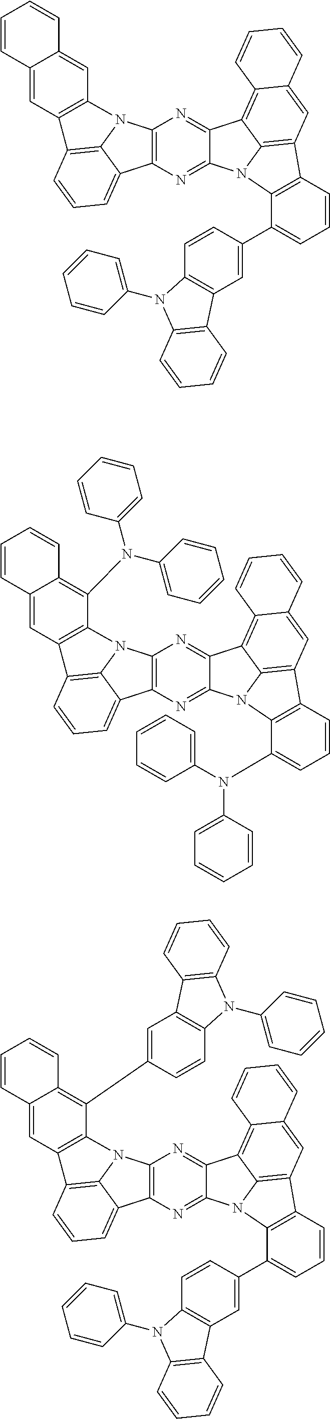

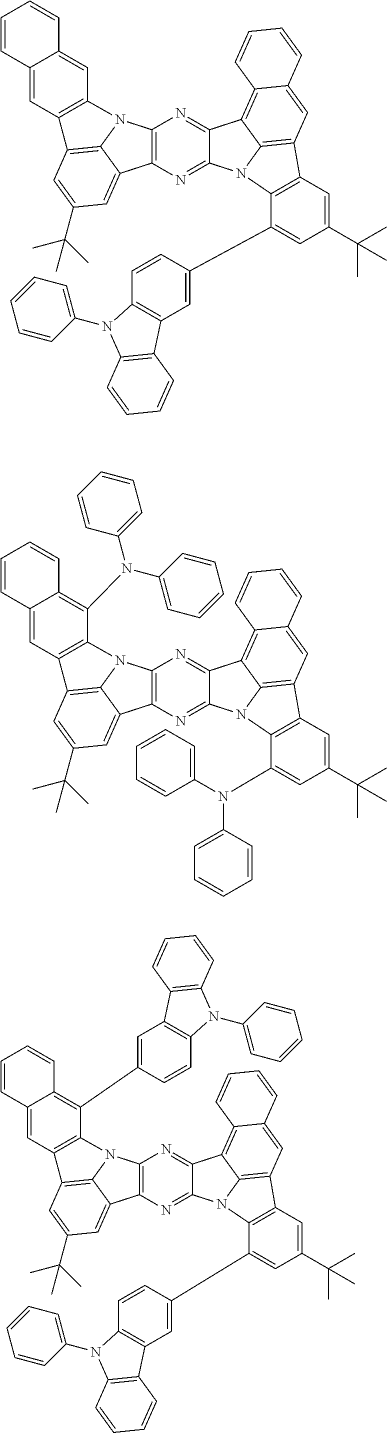 [SMILES: CC(C)(C)c1cc(-c2ccc3c(c2)c2ccccc2n3-c2ccccc2)c2c(c1)c1cc3ccccc3c3c4nc5c(nc4n2c13)c1cc(C(C)(C)C)cc2c3cc4ccccc4cc3n5c21 ; CC(C)(C)c1cc(N(c2ccccc2)c2ccccc2)c2c(c1)c1cc3ccccc3c3c4nc5c(nc4n2c13)c1cc(C(C)(C)C)cc2c3cc4ccccc4c(N(c4ccccc4)c4ccccc4)c3n5c21 ; CC(C)(C)c1cc(-c2ccc3c(c2)c2ccccc2n3-c2ccccc2)c2c(c1)c1cc3ccccc3c3c4nc5c(nc4n2c13)c1cc(C(C)(C)C)cc2c3cc4ccccc4c(-c4ccc6c(c4)c4ccccc4n6-c4ccccc4)c3n5c21]

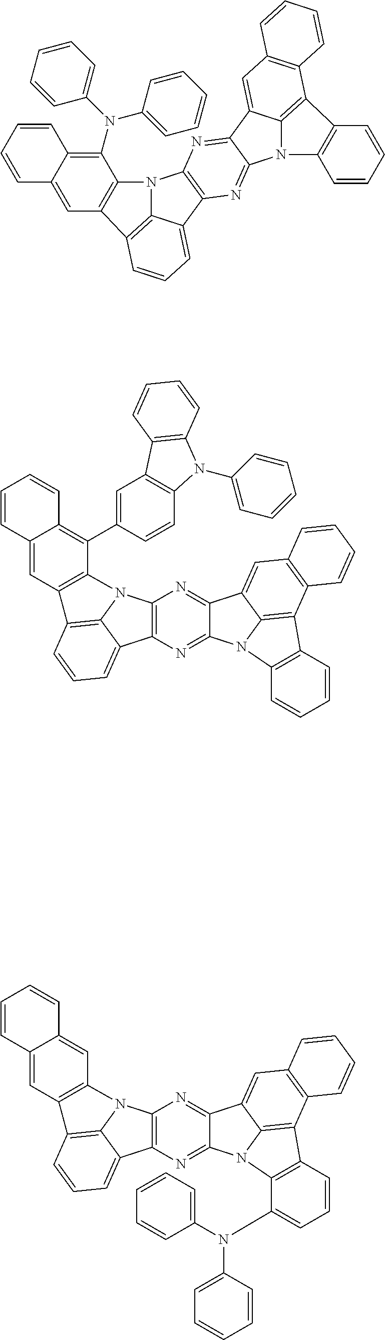 [SMILES: c1ccc(N(c2ccccc2)c2c3ccccc3cc3c4cccc5c6nc7c(nc6n(c23)c45)c2cc3ccccc3c3c4ccccc4n7c23)cc1 ; c1ccc(-n2c3ccccc3c3cc(-c4c5ccccc5cc5c6cccc7c8nc9c(nc8n(c45)c67)c4cc5ccccc5c5c6ccccc6n9c45)ccc32)cc1 ; c1ccc(N(c2ccccc2)c2cccc3c4c5ccccc5cc5c6nc7c(nc6n(c23)c54)c2cccc3c4cc5ccccc5cc4n7c32)cc1]

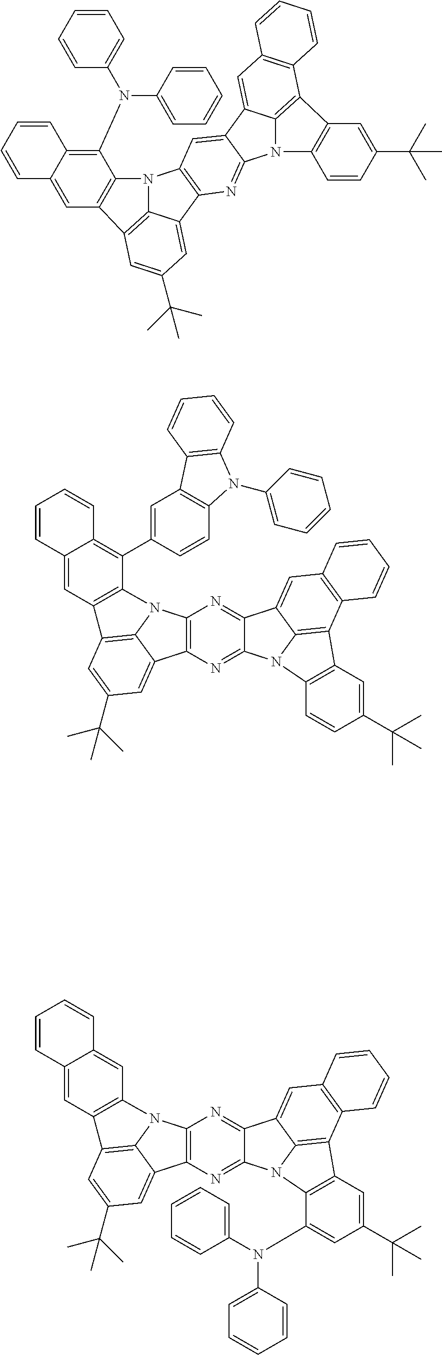 [SMILES: CC(C)(C)c1ccc2c(c1)c1c3ccccc3cc3c4cc5c(nc4n2c31)c1cc(C(C)(C)C)cc2c3cc4ccccc4c(N(c4ccccc4)c4ccccc4)c3n5c21 ; CC(C)(C)c1ccc2c(c1)c1c3ccccc3cc3c4nc5c(nc4n2c31)c1cc(C(C)(C)C)cc2c3cc4ccccc4c(-c4ccc6c(c4)c4ccccc4n6-c4ccccc4)c3n5c21 ; CC(C)(C)c1cc(N(c2ccccc2)c2ccccc2)c2c(c1)c1c3ccccc3cc3c4nc5c(nc4n2c31)c1cc(C(C)(C)C)cc2c3cc4ccccc4cc3n5c21]

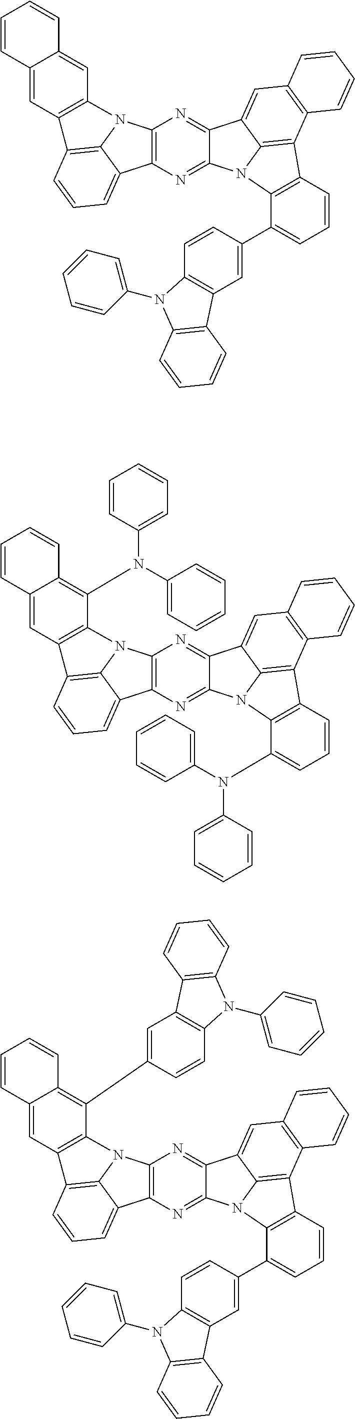

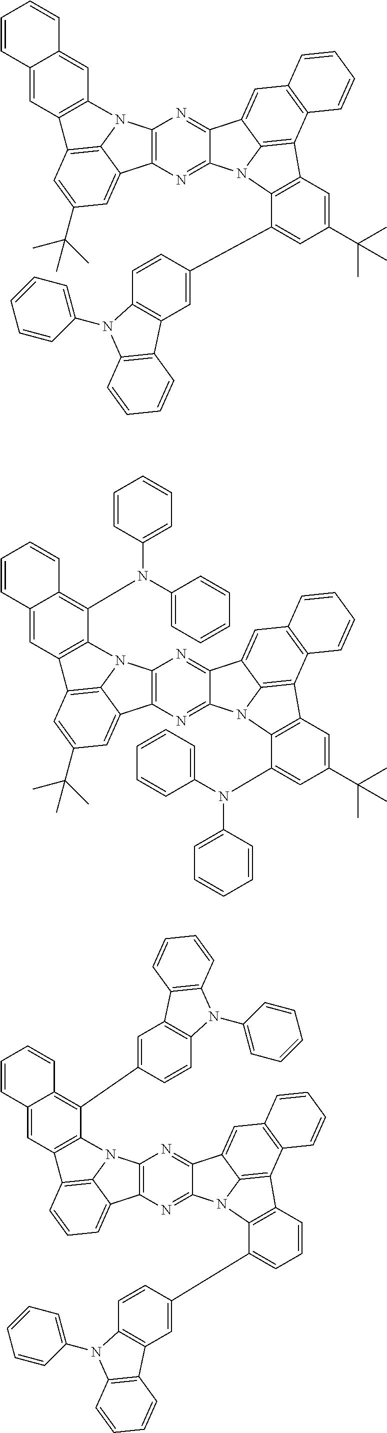 [SMILES: CC(C)(C)c1cc(-c2ccc3c(c2)c2ccccc2n3-c2ccccc2)c2c(c1)c1c3ccccc3cc3c4nc5c(nc4n2c31)c1cc(C(C)(C)C)cc2c3cc4ccccc4cc3n5c21 ; CC(C)(C)c1cc(N(c2ccccc2)c2ccccc2)c2c(c1)c1c3ccccc3cc3c4nc5c(nc4n2c31)c1cc(C(C)(C)C)cc2c3cc4ccccc4c(N(c4ccccc4)c4ccccc4)c3n5c21 ; c1ccc(-n2c3ccccc3c3cc(-c4c5ccccc5cc5c6cccc7c8nc9c(nc8n(c45)c67)c4cc5ccccc5c5c6cccc(-c7ccc8c(c7)c7ccccc7n8-c7ccccc7)c6n9c45)ccc32)cc1]

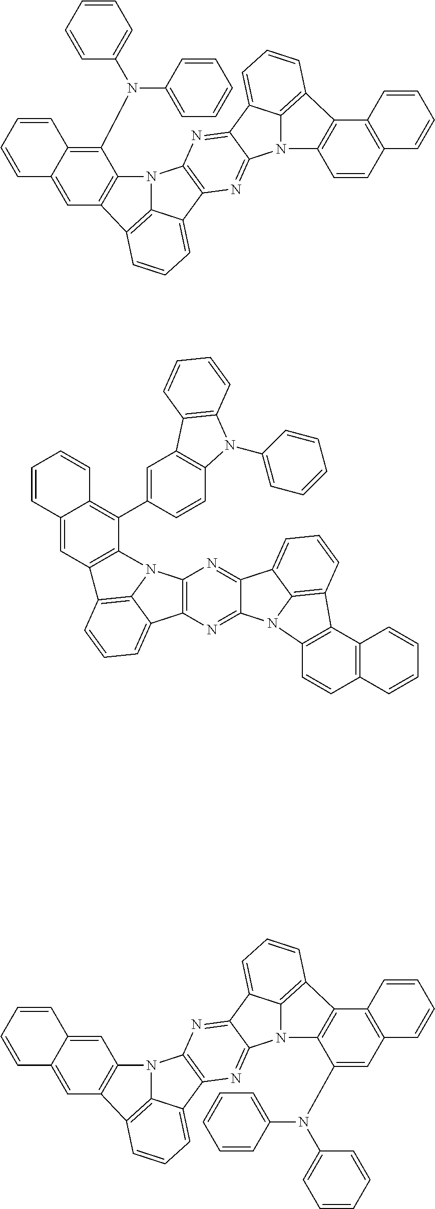 [SMILES: c1ccc(N(c2ccccc2)c2c3ccccc3cc3c4cccc5c6nc7c(nc6n(c23)c45)c2cccc3c4c5ccccc5ccc4n7c23)cc1 ; c1ccc(-n2c3ccccc3c3cc(-c4c5ccccc5cc5c6cccc7c8nc9c(nc8n(c45)c67)c4cccc5c6c7ccccc7ccc6n9c45)ccc32)cc1 ; c1ccc(N(c2ccccc2)c2cc3ccccc3c3c4cccc5c6nc7c(nc6n(c23)c54)c2cccc3c4cc5ccccc5cc4n7c32)cc1]

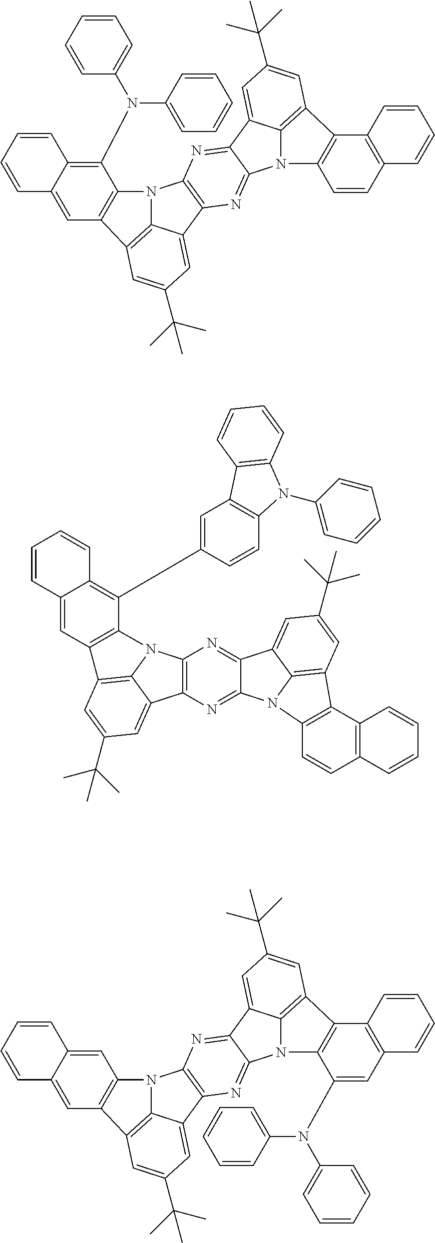 [SMILES: CC(C)(C)c1cc2c3nc4c(nc3n3c5ccc6ccccc6c5c(c1)c23)c1cc(C(C)(C)C)cc2c3cc5ccccc5c(N(c5ccccc5)c5ccccc5)c3n4c21 ; CC(C)(C)c1cc2c3nc4c(nc3n3c5ccc6ccccc6c5c(c1)c23)c1cc(C(C)(C)C)cc2c3cc5ccccc5c(-c5ccc6c(c5)c5ccccc5n6-c5ccccc5)c3n4c21 ; CC(C)(C)c1cc2c3cc4ccccc4cc3n3c4nc5c6cc(C(C)(C)C)cc7c8c9ccccc9cc(N(c9ccccc9)c9ccccc9)c8n(c5nc4c(c1)c23)c67]

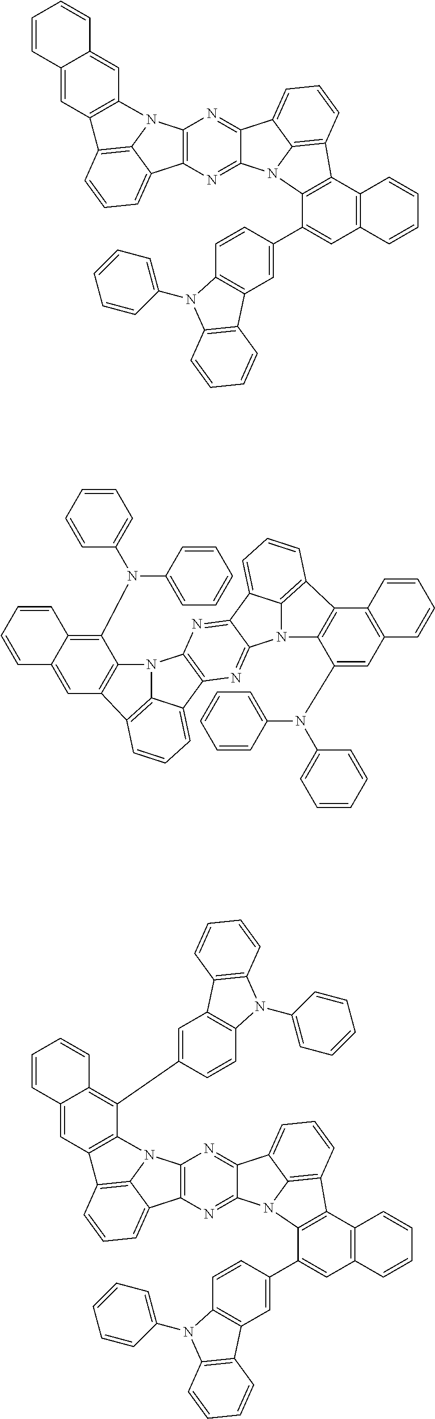 [SMILES: c1ccc(-n2c3ccccc3c3cc(-c4cc5ccccc5c5c6cccc7c8nc9c(nc8n(c45)c76)c4cccc5c6cc7ccccc7cc6n9c54)ccc32)cc1 ; c1ccc(N(c2ccccc2)c2c3ccccc3cc3c4cccc5c6nc7c(nc6n(c23)c45)c2cccc3c4c5ccccc5cc(N(c5ccccc5)c5ccccc5)c4n7c23)cc1 ; c1ccc(-n2c3ccccc3c3cc(-c4c5ccccc5cc5c6cccc7c8nc9c(nc8n(c45)c67)c4cccc5c6c7ccccc7cc(-c7ccc8c(c7)c7ccccc7n8-c7ccccc7)c6n9c45)ccc32)cc1]

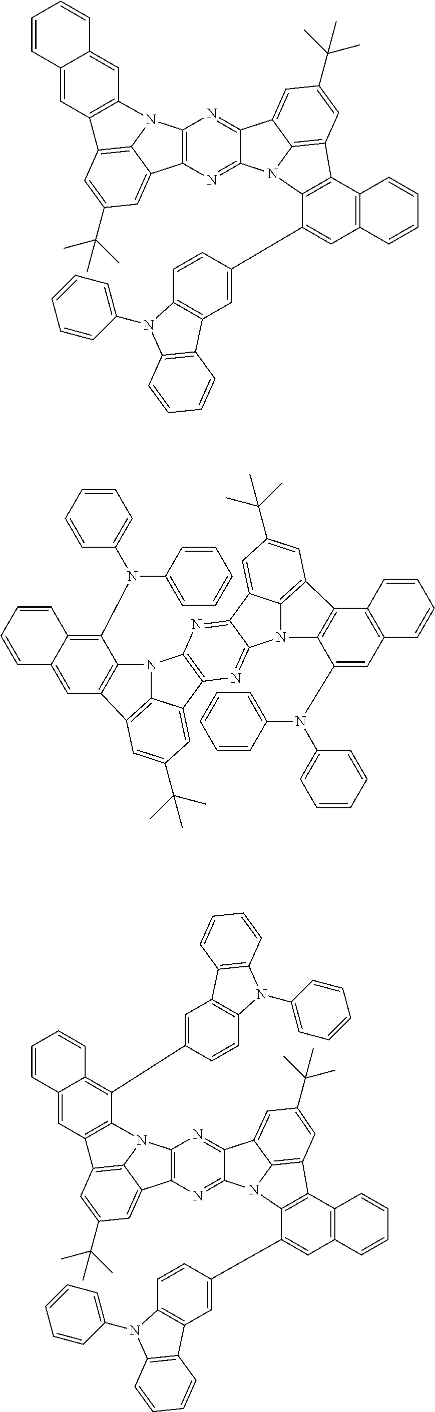 [SMILES: CC(C)(C)c1cc2c3cc4ccccc4cc3n3c4nc5c6cc(C(C)(C)C)cc7c8c9ccccc9cc(-c9ccc%10c(c9)c9ccccc9n%10-c9ccccc9)c8n(c5nc4c(c1)c23)c67 ; CC(C)(C)c1cc2c3cc4ccccc4c(N(c4ccccc4)c4ccccc4)c3n3c4nc5c6cc(C(C)(C)C)cc7c8c9ccccc9cc(N(c9ccccc9)c9ccccc9)c8n(c5nc4c(c1)c23)c67 ; CC(C)(C)c1cc2c3cc4ccccc4c(-c4ccc5c(c4)c4ccccc4n5-c4ccccc4)c3n3c4nc5c6cc(C(C)(C)C)cc7c8c9ccccc9cc(-c9ccc%10c(c9)c9ccccc9n%10-c9ccccc9)c8n(c5nc4c(c1)c23)c67]

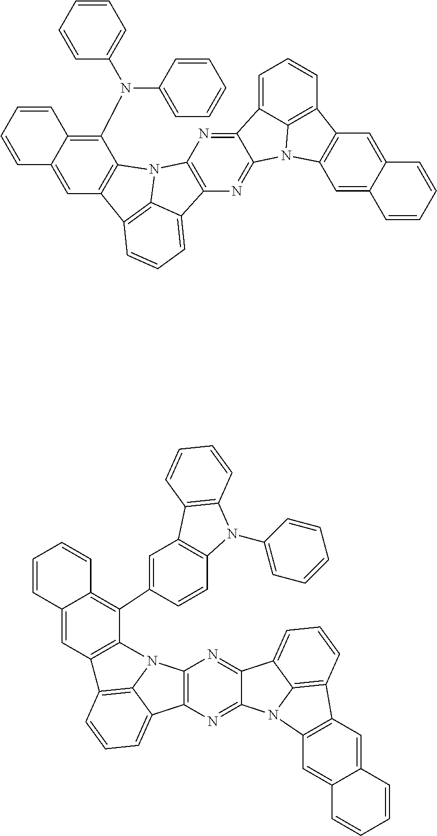 [SMILES: c1ccc(N(c2ccccc2)c2c3ccccc3cc3c4cccc5c6nc7c(nc6n(c23)c45)c2cccc3c4cc5ccccc5cc4n7c32)cc1 ; c1ccc(-n2c3ccccc3c3cc(-c4c5ccccc5cc5c6cccc7c8nc9c(nc8n(c45)c67)c4cccc5c6cc7ccccc7cc6n9c54)ccc32)cc1]

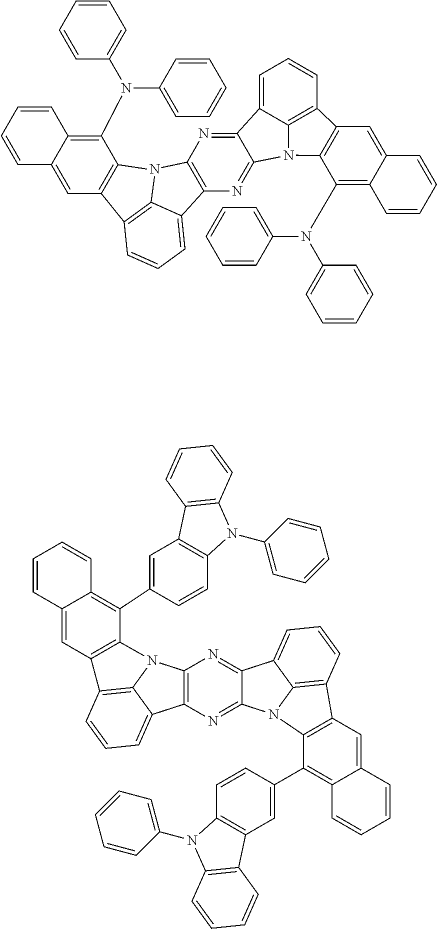 [SMILES: c1ccc(N(c2ccccc2)c2c3ccccc3cc3c4cccc5c6nc7c(nc6n(c23)c45)c2cccc3c4cc5ccccc5c(N(c5ccccc5)c5ccccc5)c4n7c32)cc1 ; c1ccc(-n2c3ccccc3c3cc(-c4c5ccccc5cc5c6cccc7c8nc9c(nc8n(c45)c67)c4cccc5c6cc7ccccc7c(-c7ccc8c(c7)c7ccccc7n8-c7ccccc7)c6n9c54)ccc32)cc1]

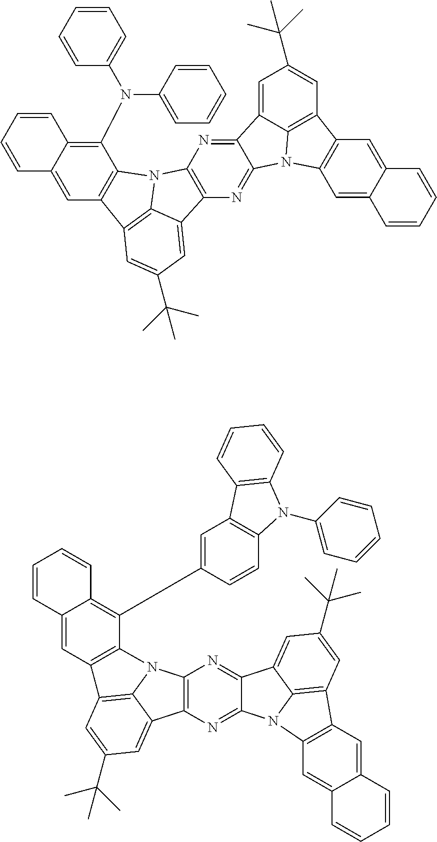 [SMILES: CC(C)(C)c1cc2c3cc4ccccc4cc3n3c4nc5c6cc(C(C)(C)C)cc7c8cc9ccccc9c(N(c9ccccc9)c9ccccc9)c8n(c5nc4c(c1)c23)c76 ; CC(C)(C)c1cc2c3cc4ccccc4cc3n3c4nc5c6cc(C(C)(C)C)cc7c8cc9ccccc9c(-c9ccc%10c(c9)c9ccccc9n%10-c9ccccc9)c8n(c5nc4c(c1)c23)c76]

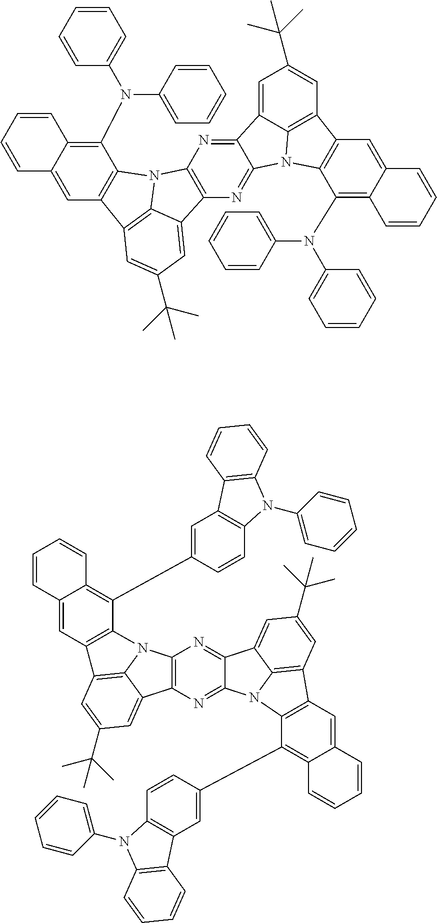 [SMILES: CC(C)(C)c1cc2c3cc4ccccc4c(N(c4ccccc4)c4ccccc4)c3n3c4nc5c6cc(C(C)(C)C)cc7c8cc9ccccc9c(N(c9ccccc9)c9ccccc9)c8n(c5nc4c(c1)c23)c76 ; CC(C)(C)c1cc2c3cc4ccccc4c(-c4ccc5c(c4)c4ccccc4n5-c4ccccc4)c3n3c4nc5c6cc(C(C)(C)C)cc7c8cc9ccccc9c(-c9ccc%10c(c9)c9ccccc9n%10-c9ccccc9)c8n(c5nc4c(c1)c23)c76]

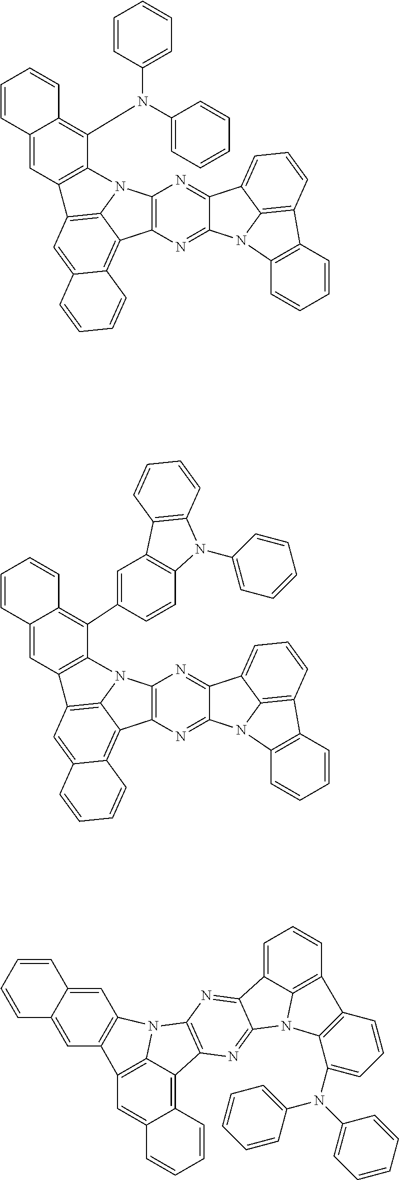 [SMILES: c1ccc(N(c2ccccc2)c2c3ccccc3cc3c4cc5ccccc5c5c6nc7c(nc6n(c23)c45)c2cccc3c4ccccc4n7c32)cc1 ; c1ccc(-n2c3ccccc3c3cc(-c4c5ccccc5cc5c6cc7ccccc7c7c8nc9c(nc8n(c45)c67)c4cccc5c6ccccc6n9c54)ccc32)cc1 ; c1ccc(N(c2ccccc2)c2cccc3c4cccc5c6nc7c(nc6n(c23)c45)c2c3ccccc3cc3c4cc5ccccc5cc4n7c32)cc1]

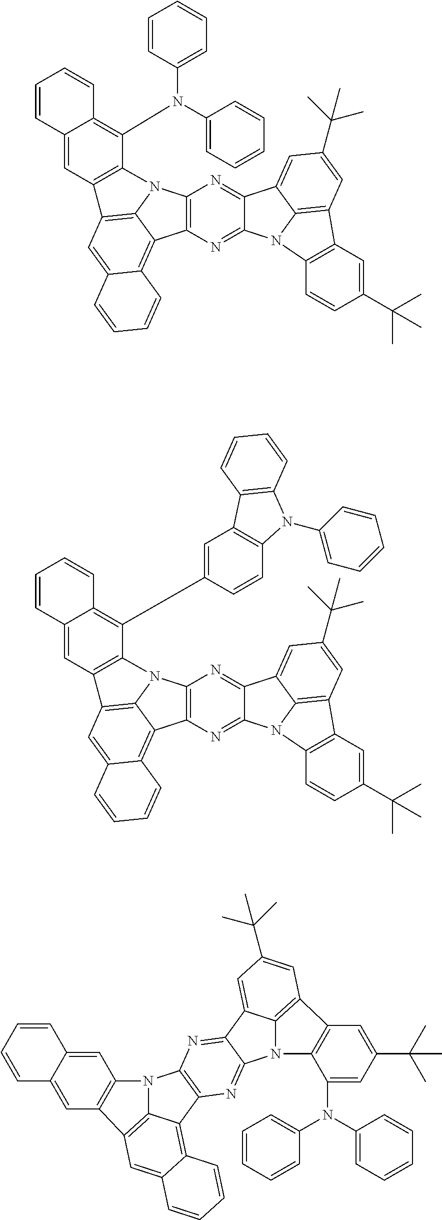 [SMILES: CC(C)(C)c1ccc2c(c1)c1cc(C(C)(C)C)cc3c4nc5c(nc4n2c13)c1c2ccccc2cc2c3cc4ccccc4c(N(c4ccccc4)c4ccccc4)c3n5c21 ; CC(C)(C)c1ccc2c(c1)c1cc(C(C)(C)C)cc3c4nc5c(nc4n2c13)c1c2ccccc2cc2c3cc4ccccc4c(-c4ccc6c(c4)c4ccccc4n6-c4ccccc4)c3n5c21 ; CC(C)(C)c1cc(N(c2ccccc2)c2ccccc2)c2c(c1)c1cc(C(C)(C)C)cc3c4nc5c(nc4n2c13)c1c2ccccc2cc2c3cc4ccccc4cc3n5c21]

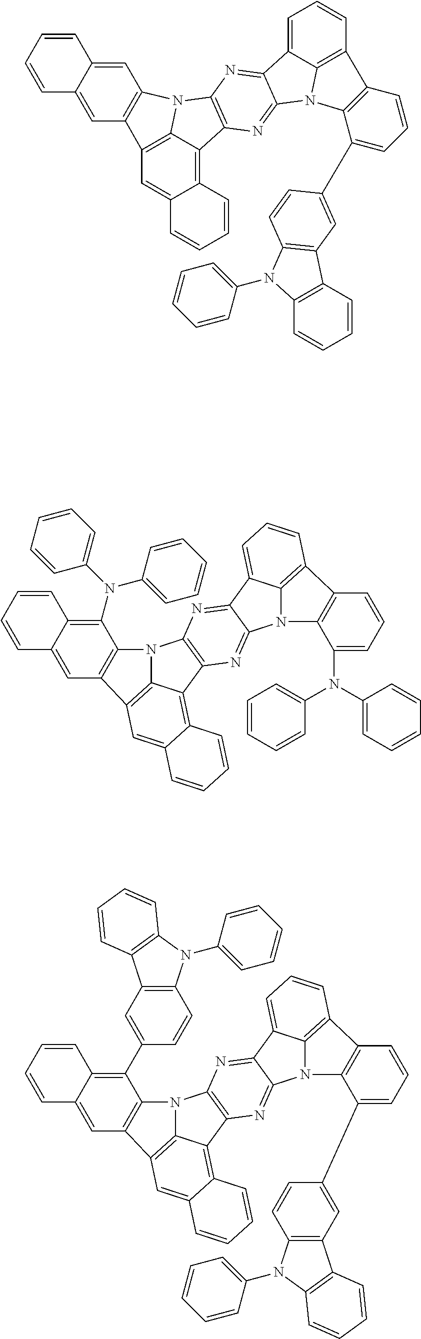 [SMILES: c1ccc(-n2c3ccccc3c3cc(-c4cccc5c6cccc7c8nc9c(nc8n(c45)c67)c4c5ccccc5cc5c6cc7ccccc7cc6n9c54)ccc32)cc1 ; c1ccc(N(c2ccccc2)c2cccc3c4cccc5c6nc7c(nc6n(c23)c45)c2c3ccccc3cc3c4cc5ccccc5c(N(c5ccccc5)c5ccccc5)c4n7c32)cc1 ; c1ccc(-n2c3ccccc3c3cc(-c4cccc5c6cccc7c8nc9c(nc8n(c45)c67)c4c5ccccc5cc5c6cc7ccccc7c(-c7ccc8c(c7)c7ccccc7n8-c7ccccc7)c6n9c54)ccc32)cc1]

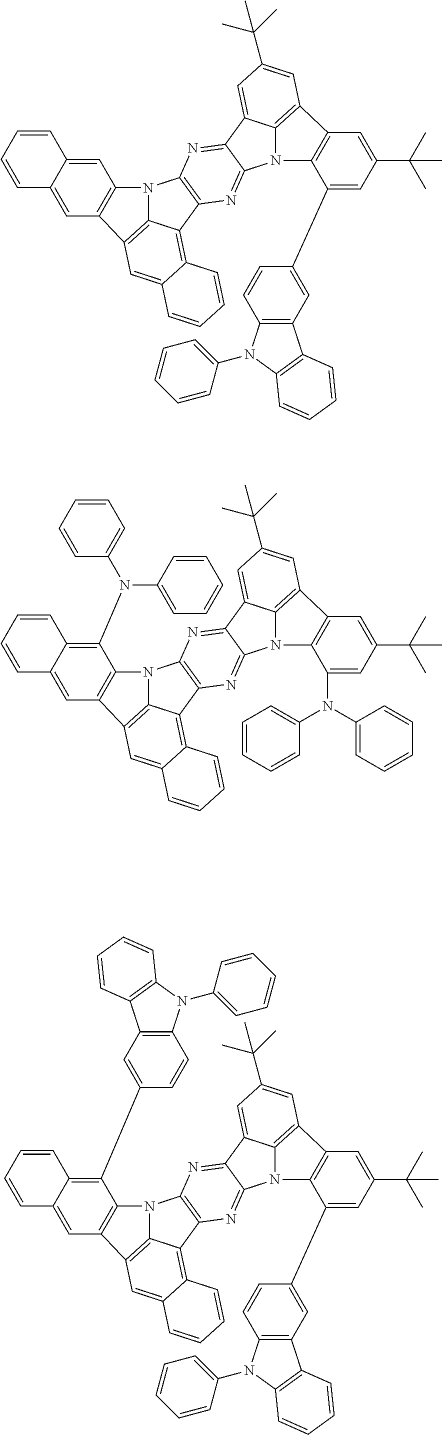 [SMILES: CC(C)(C)c1cc(-c2ccc3c(c2)c2ccccc2n3-c2ccccc2)c2c(c1)c1cc(C(C)(C)C)cc3c4nc5c(nc4n2c13)c1c2ccccc2cc2c3cc4ccccc4cc3n5c21 ; CC(C)(C)c1cc(N(c2ccccc2)c2ccccc2)c2c(c1)c1cc(C(C)(C)C)cc3c4nc5c(nc4n2c13)c1c2ccccc2cc2c3cc4ccccc4c(N(c4ccccc4)c4ccccc4)c3n5c21 ; CC(C)(C)c1cc(-c2ccc3c(c2)c2ccccc2n3-c2ccccc2)c2c(c1)c1cc(C(C)(C)C)cc3c4nc5c(nc4n2c13)c1c2ccccc2cc2c3cc4ccccc4c(-c4ccc6c(c4)c4ccccc4n6-c4ccccc4)c3n5c21]

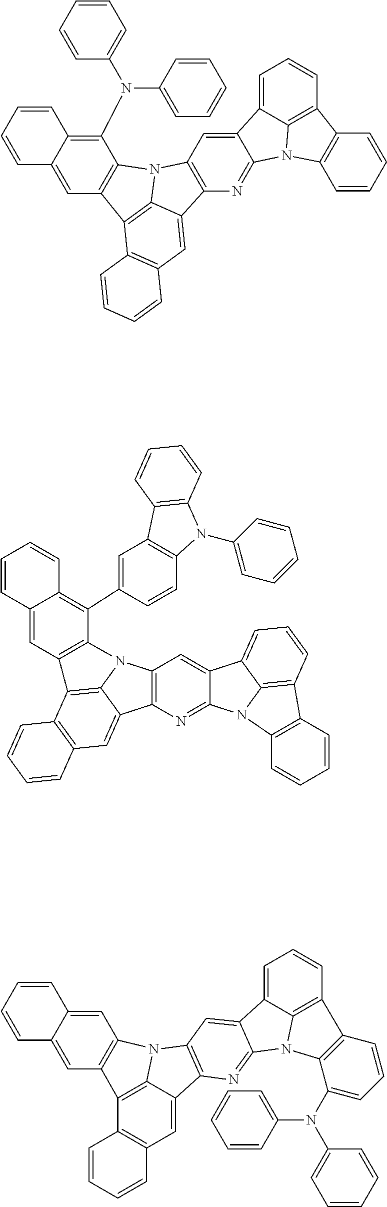 [SMILES: c1ccc(N(c2ccccc2)c2c3ccccc3cc3c4c5ccccc5cc5c6nc7c(cc6n(c23)c54)c2cccc3c4ccccc4n7c32)cc1 ; c1ccc(-n2c3ccccc3c3cc(-c4c5ccccc5cc5c6c7ccccc7cc7c8nc9c(cc8n(c45)c76)c4cccc5c6ccccc6n9c54)ccc32)cc1 ; c1ccc(N(c2ccccc2)c2cccc3c4cccc5c6cc7c(nc6n(c23)c54)c2cc3ccccc3c3c4cc5ccccc5cc4n7c23)cc1]

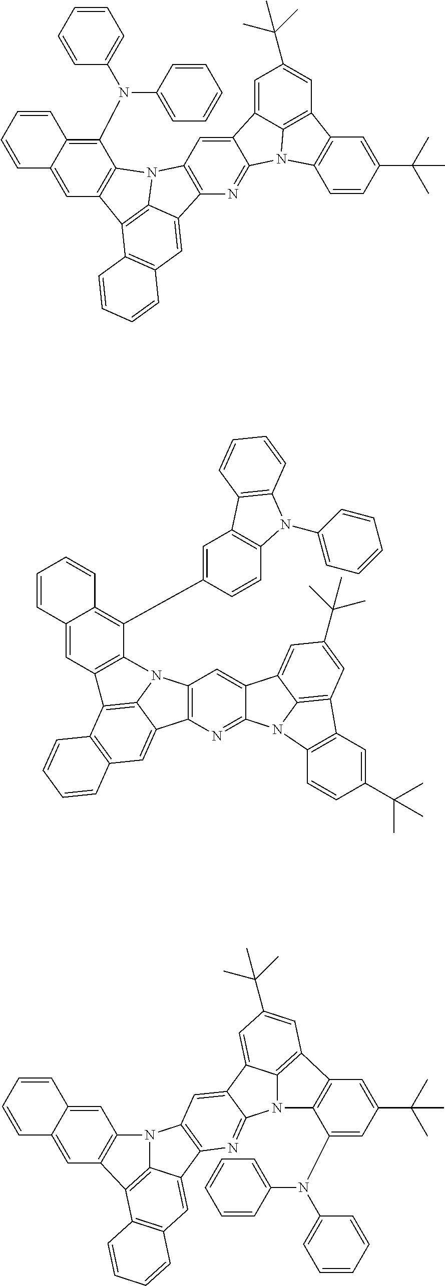 [SMILES: CC(C)(C)c1ccc2c(c1)c1cc(C(C)(C)C)cc3c4cc5c(nc4n2c13)c1cc2ccccc2c2c3cc4ccccc4c(N(c4ccccc4)c4ccccc4)c3n5c12 ; CC(C)(C)c1ccc2c(c1)c1cc(C(C)(C)C)cc3c4cc5c(nc4n2c13)c1cc2ccccc2c2c3cc4ccccc4c(-c4ccc6c(c4)c4ccccc4n6-c4ccccc4)c3n5c12 ; CC(C)(C)c1cc(N(c2ccccc2)c2ccccc2)c2c(c1)c1cc(C(C)(C)C)cc3c4cc5c(nc4n2c31)c1cc2ccccc2c2c3cc4ccccc4cc3n5c12]

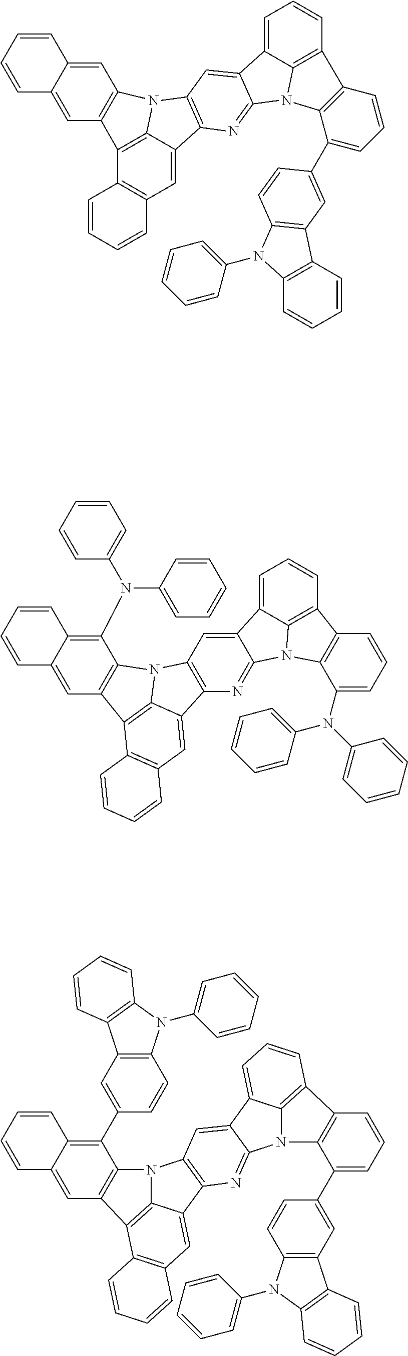 [SMILES: c1ccc(-n2c3ccccc3c3cc(-c4cccc5c6cccc7c8cc9c(nc8n(c45)c76)c4cc5ccccc5c5c6cc7ccccc7cc6n9c45)ccc32)cc1 ; c1ccc(N(c2ccccc2)c2c3ccccc3cc3c4c5ccccc5cc5c6nc7c(cc6n(c23)c54)c2cccc3c4cccc(N(c5ccccc5)c5ccccc5)c4n7c23)cc1 ; c1ccc(-n2c3ccccc3c3cc(-c4c5ccccc5cc5c6c7ccccc7cc7c8nc9c(cc8n(c45)c76)c4cccc5c6cccc(-c7ccc8c(c7)c7ccccc7n8-c7ccccc7)c6n9c45)ccc32)cc1]

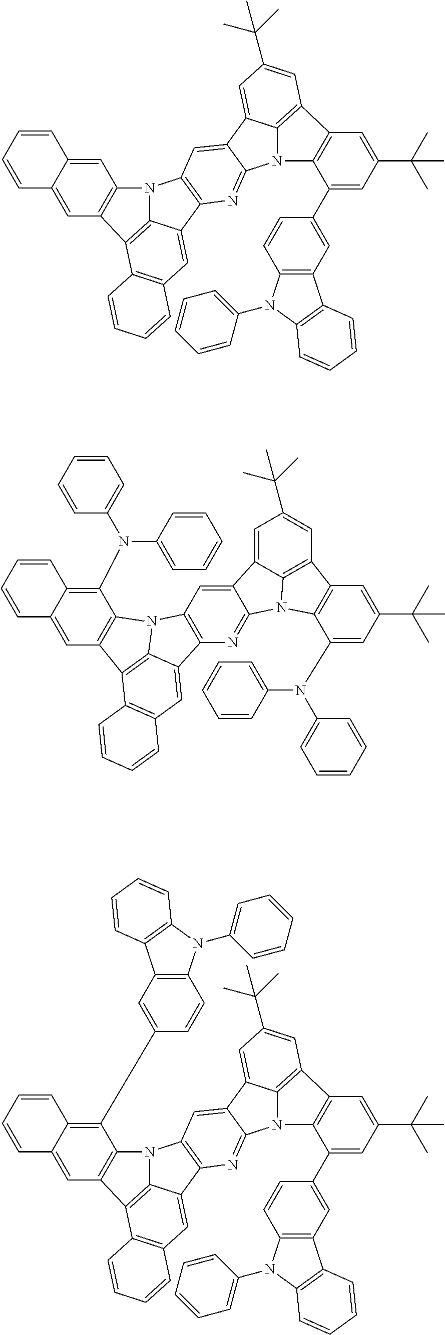 [SMILES: CC(C)(C)c1cc(-c2ccc3c(c2)c2ccccc2n3-c2ccccc2)c2c(c1)c1cc(C(C)(C)C)cc3c4cc5c(nc4n2c31)c1cc2ccccc2c2c3cc4ccccc4cc3n5c12 ; CC(C)(C)c1cc(N(c2ccccc2)c2ccccc2)c2c(c1)c1cc(C(C)(C)C)cc3c4cc5c(nc4n2c31)c1cc2ccccc2c2c3cc4ccccc4c(N(c4ccccc4)c4ccccc4)c3n5c12 ; CC(C)(C)c1cc(-c2ccc3c(c2)c2ccccc2n3-c2ccccc2)c2c(c1)c1cc(C(C)(C)C)cc3c4cc5c(nc4n2c31)c1cc2ccccc2c2c3cc4ccccc4c(-c4ccc6c(c4)c4ccccc4n6-c4ccccc4)c3n5c12]

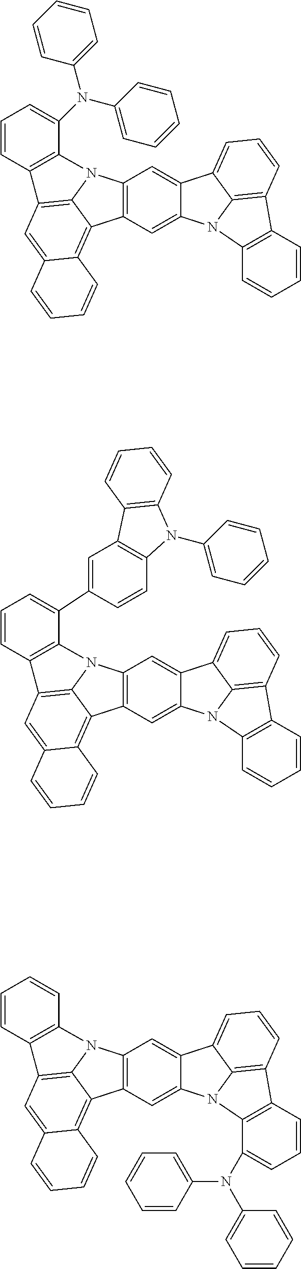 [SMILES: c1ccc(N(c2ccccc2)c2cccc3c4cc5ccccc5c5c6cc7c(cc6n(c23)c45)c2cccc3c4ccccc4n7c32)cc1 ; c1ccc(-n2c3ccccc3c3cc(-c4cccc5c6cc7ccccc7c7c8cc9c(cc8n(c45)c67)c4cccc5c6ccccc6n9c54)ccc32)cc1 ; c1ccc(N(c2ccccc2)c2cccc3c4cccc5c6cc7c(cc6n(c23)c54)c2c3ccccc3cc3c4ccccc4n7c32)cc1]

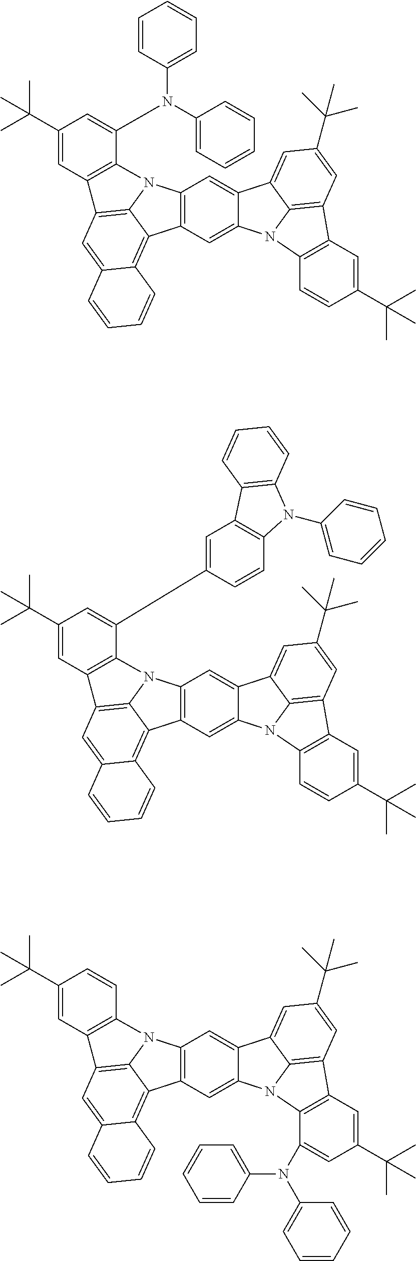

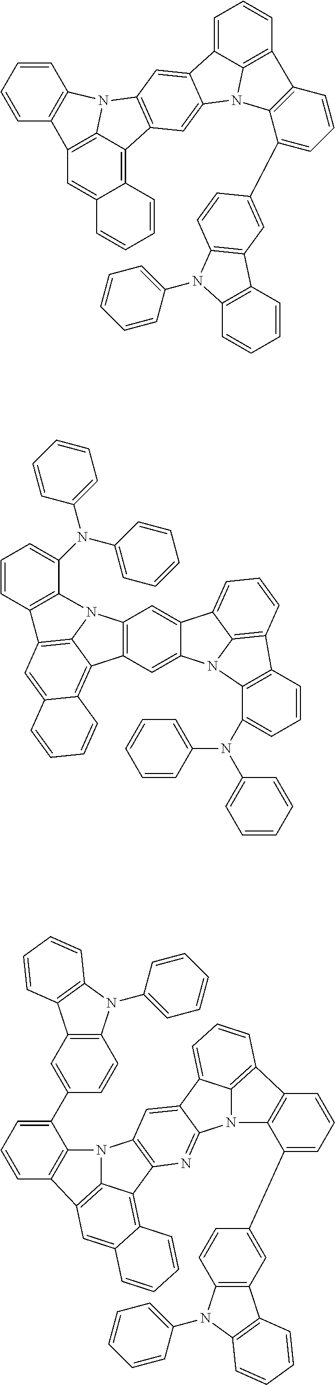 [SMILES: c1ccc(-n2c3ccccc3c3cc(-c4cccc5c6cccc7c8cc9c(cc8n(c45)c76)c4c5ccccc5cc5c6ccccc6n9c54)ccc32)cc1 ; c1ccc(N(c2ccccc2)c2cccc3c4cccc5c6cc7c(cc6n(c23)c54)c2c3ccccc3cc3c4cccc(N(c5ccccc5)c5ccccc5)c4n7c32)cc1 ; c1ccc(-n2c3ccccc3c3cc(-c4cccc5c6cc7ccccc7c7c8nc9c(cc8n(c45)c67)c4cccc5c6cccc(-c7ccc8c(c7)c7ccccc7n8-c7ccccc7)c6n9c45)ccc32)cc1]

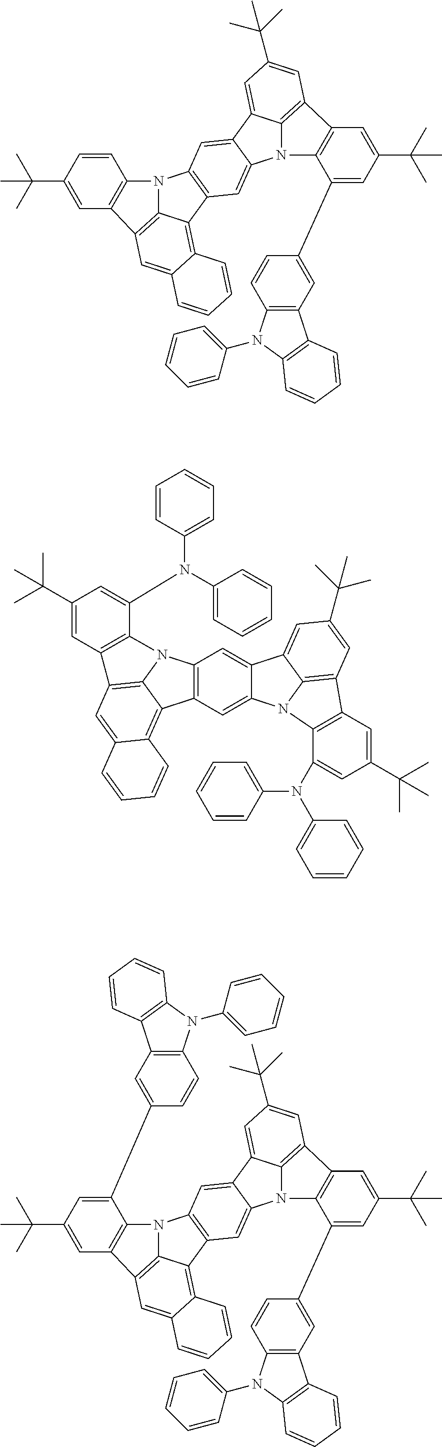

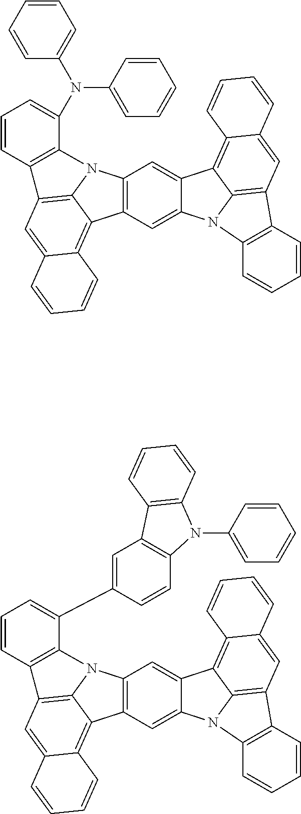 [SMILES: c1ccc(N(c2ccccc2)c2cccc3c4cc5ccccc5c5c6cc7c(cc6n(c23)c45)c2c3ccccc3cc3c4ccccc4n7c32)cc1 ; c1ccc(-n2c3ccccc3c3cc(-c4cccc5c6cc7ccccc7c7c8cc9c(cc8n(c45)c67)c4c5ccccc5cc5c6ccccc6n9c54)ccc32)cc1]

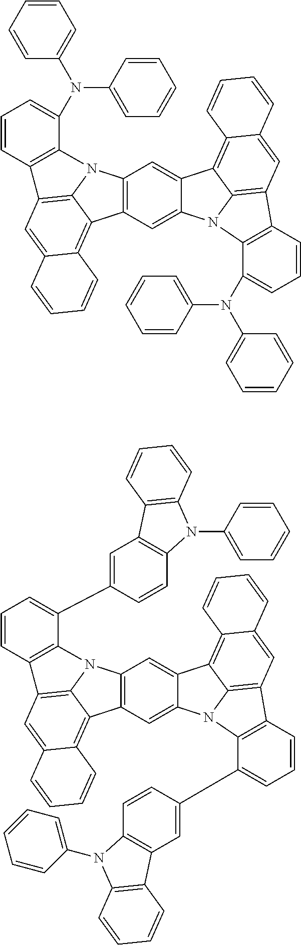 [SMILES: c1ccc(N(c2ccccc2)c2cccc3c4cc5ccccc5c5c6cc7c(cc6n(c23)c45)c2c3ccccc3cc3c4cccc(N(c5ccccc5)c5ccccc5)c4n7c32)cc1 ; c1ccc(-n2c3ccccc3c3cc(-c4cccc5c6cc7ccccc7c7c8cc9c(cc8n(c45)c67)c4c5ccccc5cc5c6cccc(-c7ccc8c(c7)c7ccccc7n8-c7ccccc7)c6n9c54)ccc32)cc1]

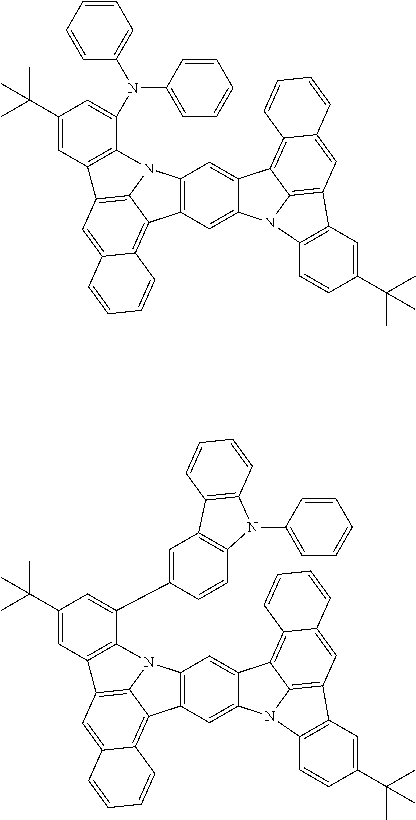

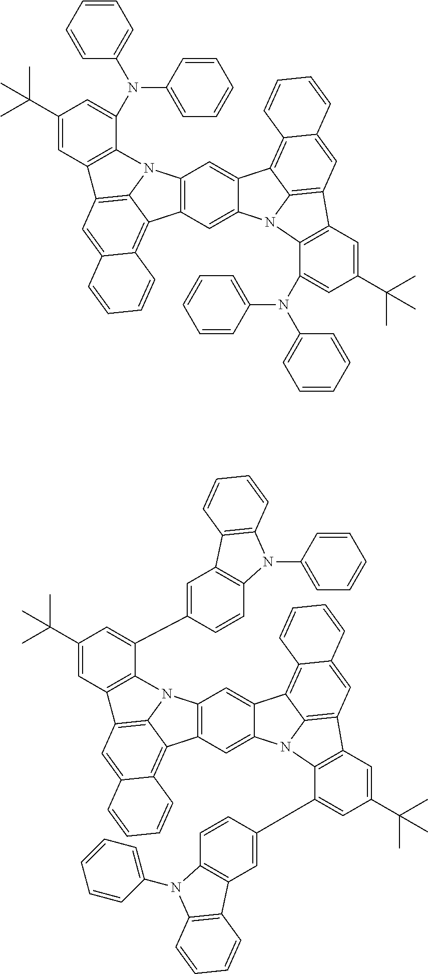

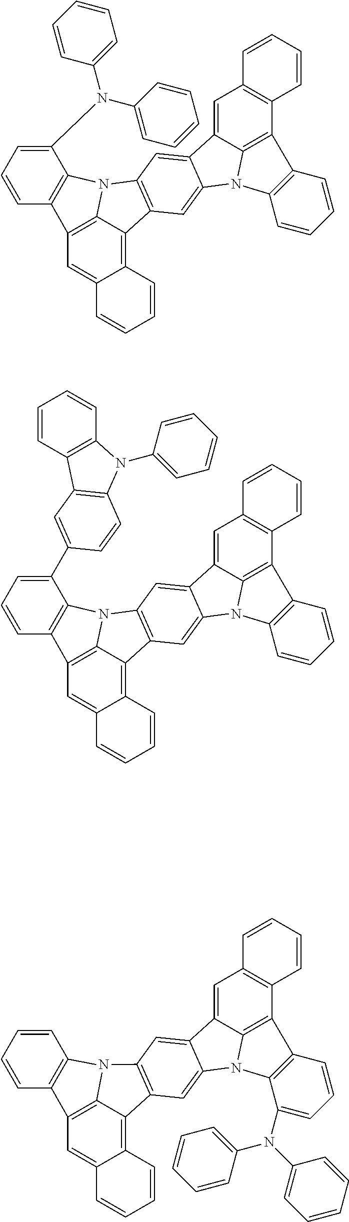 [SMILES: c1ccc(N(c2ccccc2)c2cccc3c4cc5ccccc5c5c6cc7c(cc6n(c23)c45)c2cc3ccccc3c3c4ccccc4n7c23)cc1 ; c1ccc(-n2c3ccccc3c3cc(-c4cccc5c6cc7ccccc7c7c8cc9c(cc8n(c45)c67)c4cc5ccccc5c5c6ccccc6n9c45)ccc32)cc1 ; c1ccc(N(c2ccccc2)c2cccc3c4c5ccccc5cc5c6cc7c(cc6n(c23)c54)c2c3ccccc3cc3c4ccccc4n7c32)cc1]

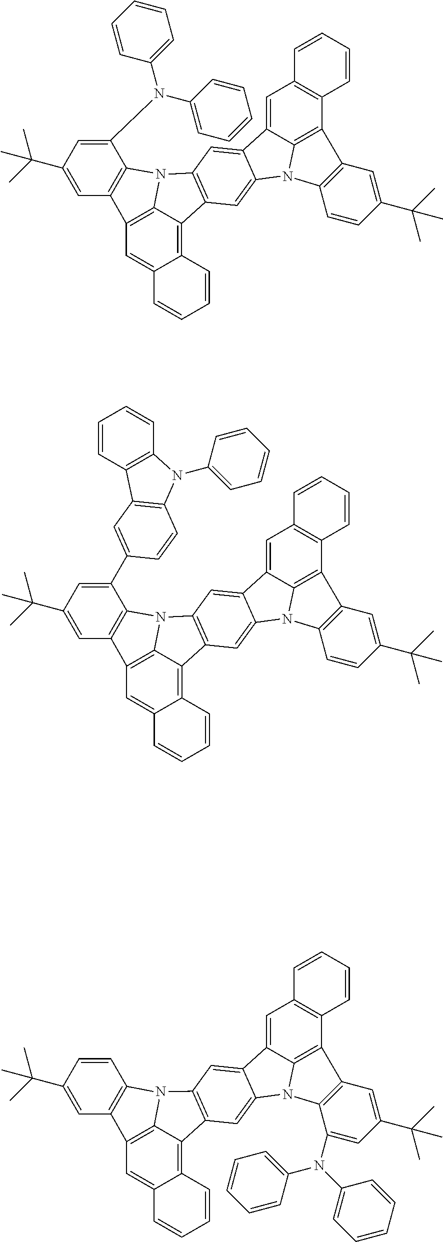

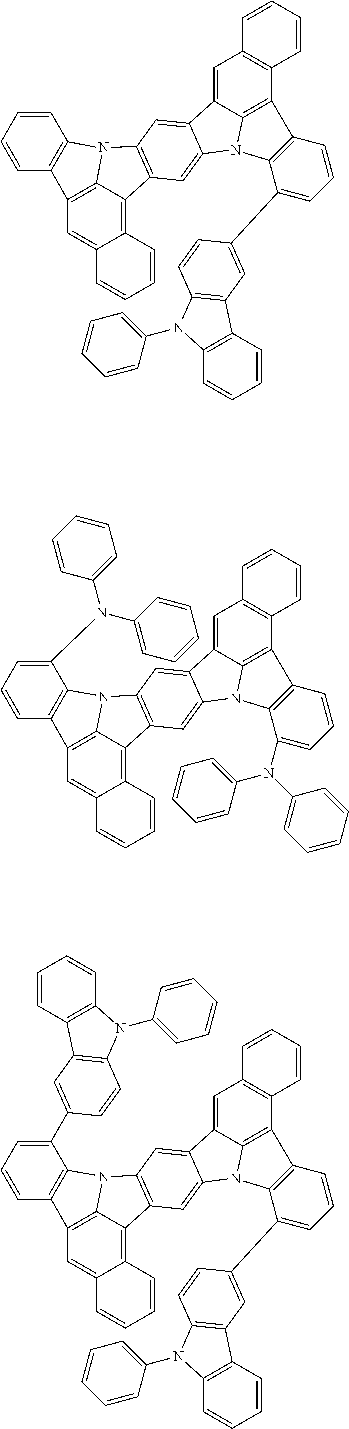 [SMILES: c1ccc(-n2c3ccccc3c3cc(-c4cccc5c6c7ccccc7cc7c8cc9c(cc8n(c45)c76)c4c5ccccc5cc5c6ccccc6n9c54)ccc32)cc1 ; c1ccc(N(c2ccccc2)c2cccc3c4cc5ccccc5c5c6cc7c(cc6n(c23)c45)c2cc3ccccc3c3c4cccc(N(c5ccccc5)c5ccccc5)c4n7c23)cc1 ; c1ccc(-n2c3ccccc3c3cc(-c4cccc5c6cc7ccccc7c7c8cc9c(cc8n(c45)c67)c4cc5ccccc5c5c6cccc(-c7ccc8c(c7)c7ccccc7n8-c7ccccc7)c6n9c45)ccc32)cc1]

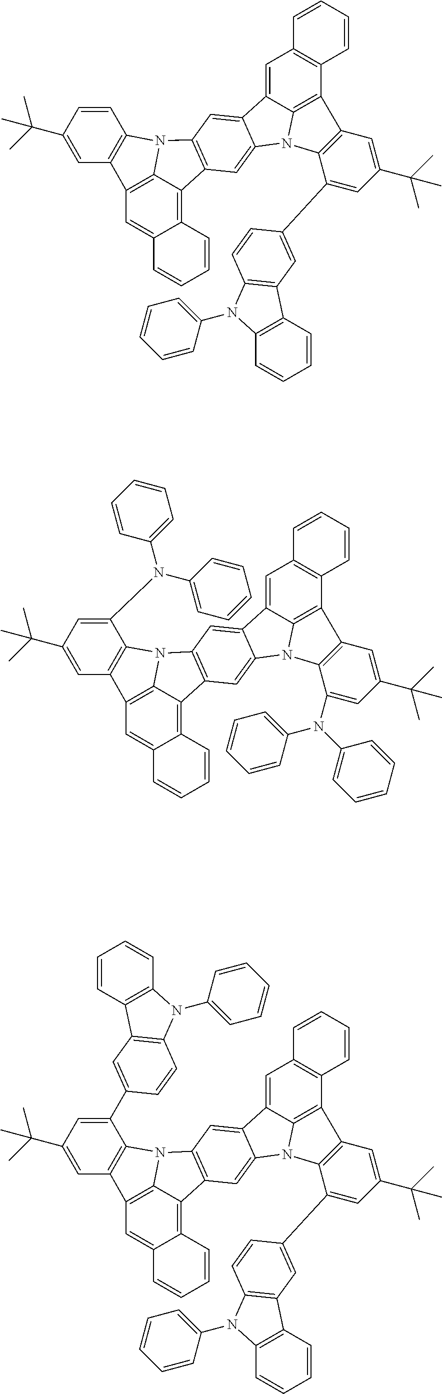

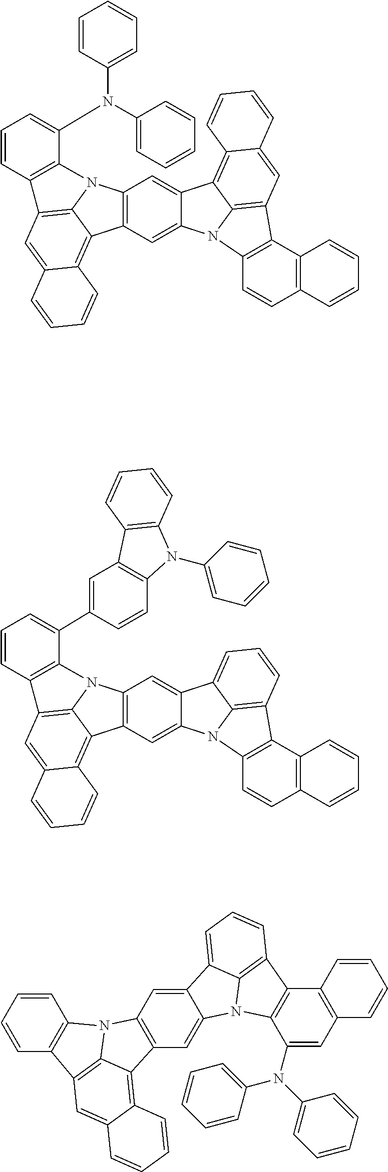 [SMILES: c1ccc(N(c2ccccc2)c2cccc3c4cc5ccccc5c5c6cc7c(cc6n(c23)c45)c2c3ccccc3cc3c4c5ccccc5ccc4n7c32)cc1 ; c1ccc(-n2c3ccccc3c3cc(-c4cccc5c6cc7ccccc7c7c8cc9c(cc8n(c45)c67)c4cccc5c6c7ccccc7ccc6n9c45)ccc32)cc1 ; c1ccc(N(c2ccccc2)c2cc3ccccc3c3c4cccc5c6cc7c(cc6n(c23)c54)c2c3ccccc3cc3c4ccccc4n7c32)cc1]

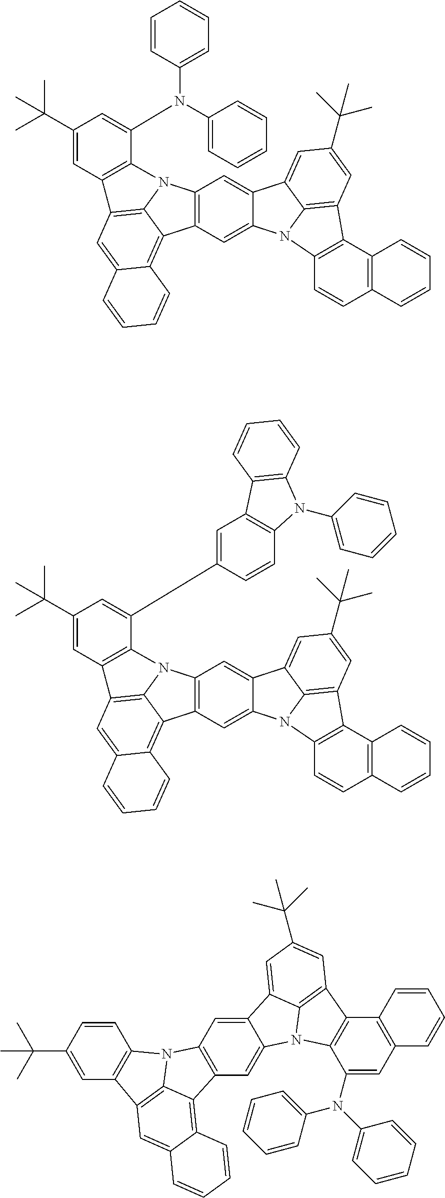 [SMILES: CC(C)(C)c1cc(N(c2ccccc2)c2ccccc2)c2c(c1)c1cc3ccccc3c3c4cc5c(cc4n2c13)c1cc(C(C)(C)C)cc2c3c4ccccc4ccc3n5c12 ; CC(C)(C)c1cc(-c2ccc3c(c2)c2ccccc2n3-c2ccccc2)c2c(c1)c1cc3ccccc3c3c4cc5c(cc4n2c13)c1cc(C(C)(C)C)cc2c3c4ccccc4ccc3n5c12 ; CC(C)(C)c1ccc2c(c1)c1cc3ccccc3c3c4cc5c(cc4n2c13)c1cc(C(C)(C)C)cc2c3c4ccccc4cc(N(c4ccccc4)c4ccccc4)c3n5c12]

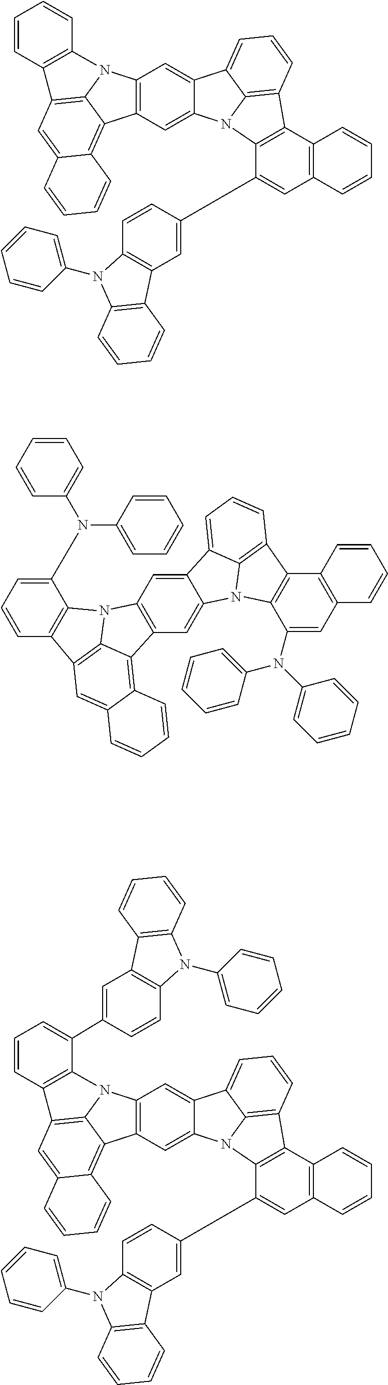 [SMILES: c1ccc(-n2c3ccccc3c3cc(-c4cc5ccccc5c5c6cccc7c8cc9c(cc8n(c45)c76)c4c5ccccc5cc5c6ccccc6n9c54)ccc32)cc1 ; c1ccc(N(c2ccccc2)c2cc3ccccc3c3c4cccc5c6cc7c(cc6n(c23)c54)c2c3ccccc3cc3c4cccc(N(c5ccccc5)c5ccccc5)c4n7c32)cc1 ; c1ccc(-n2c3ccccc3c3cc(-c4cc5ccccc5c5c6cccc7c8cc9c(cc8n(c45)c76)c4c5ccccc5cc5c6cccc(-c7ccc8c(c7)c7ccccc7n8-c7ccccc7)c6n9c54)ccc32)cc1]

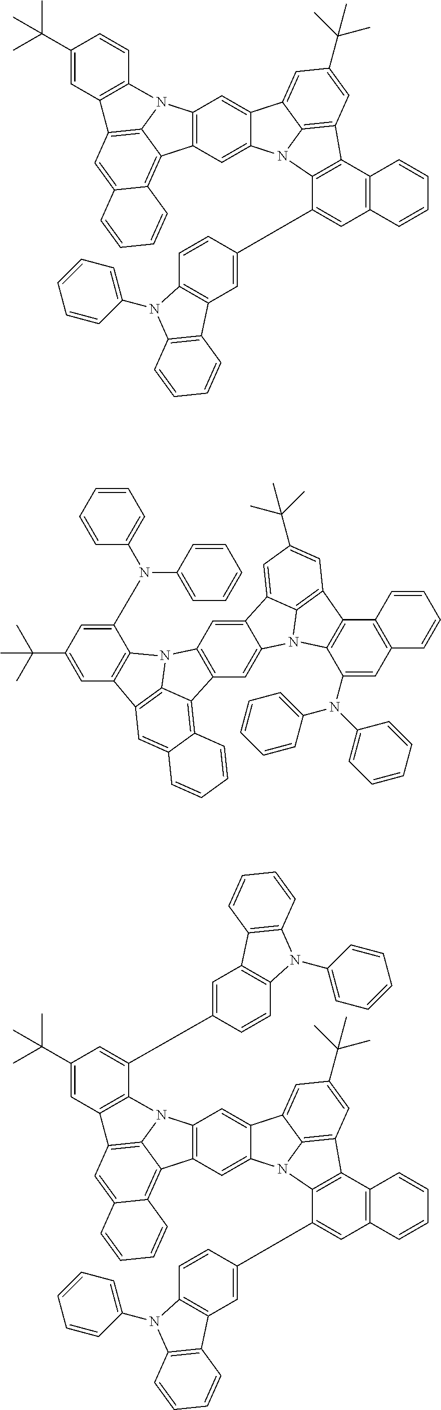 [SMILES: CC(C)(C)c1ccc2c(c1)c1cc3ccccc3c3c4cc5c(cc4n2c13)c1cc(C(C)(C)C)cc2c3c4ccccc4cc(-c4ccc6c(c4)c4ccccc4n6-c4ccccc4)c3n5c12 ; CC(C)(C)c1cc(N(c2ccccc2)c2ccccc2)c2c(c1)c1cc3ccccc3c3c4cc5c(cc4n2c13)c1cc(C(C)(C)C)cc2c3c4ccccc4cc(N(c4ccccc4)c4ccccc4)c3n5c12 ; CC(C)(C)c1cc(-c2ccc3c(c2)c2ccccc2n3-c2ccccc2)c2c(c1)c1cc3ccccc3c3c4cc5c(cc4n2c13)c1cc(C(C)(C)C)cc2c3c4ccccc4cc(-c4ccc6c(c4)c4ccccc4n6-c4ccccc4)c3n5c12]

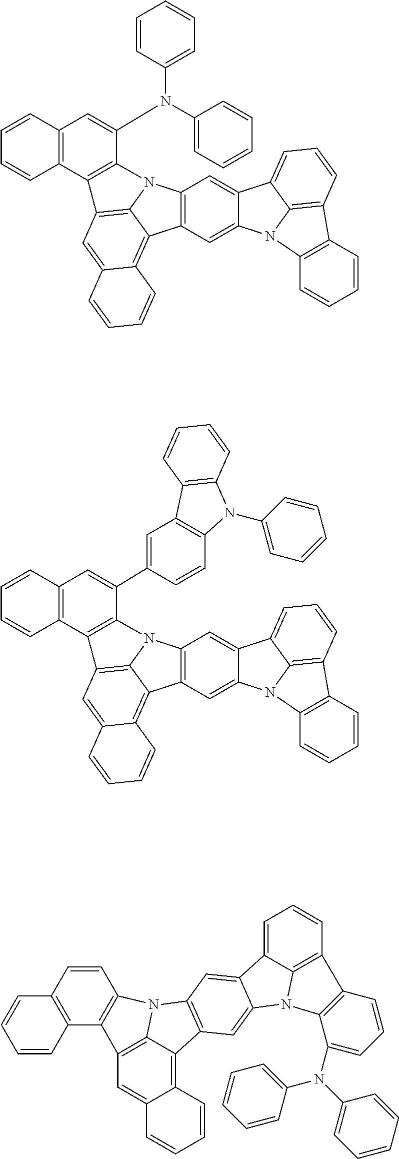 [SMILES: c1ccc(N(c2ccccc2)c2cc3ccccc3c3c4cc5ccccc5c5c6cc7c(cc6n(c23)c45)c2cccc3c4ccccc4n7c32)cc1 ; c1ccc(-n2c3ccccc3c3cc(-c4cc5ccccc5c5c6cc7ccccc7c7c8cc9c(cc8n(c45)c67)c4cccc5c6ccccc6n9c54)ccc32)cc1 ; c1ccc(N(c2ccccc2)c2cccc3c4cccc5c6cc7c(cc6n(c23)c54)c2c3ccccc3cc3c4c5ccccc5ccc4n7c32)cc1]

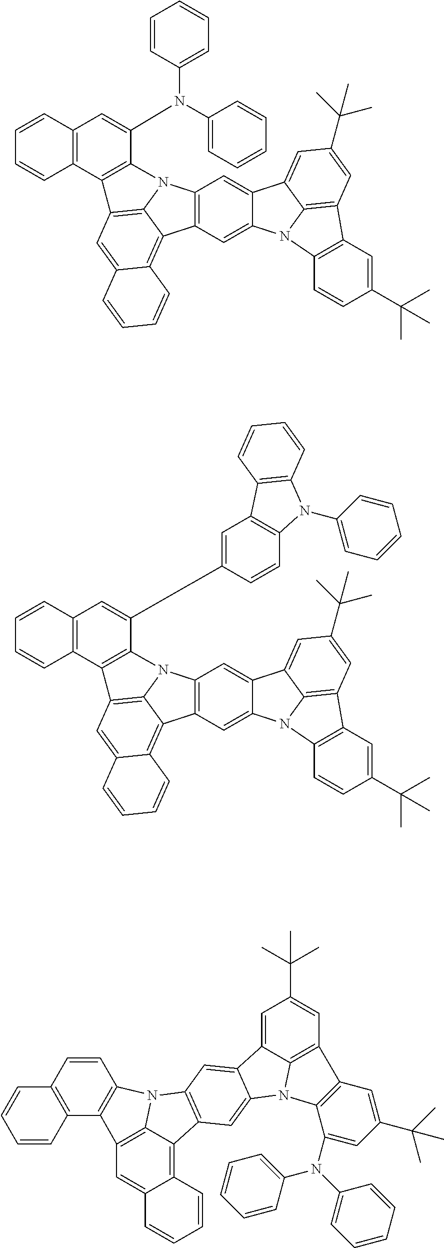 [SMILES: CC(C)(C)c1ccc2c(c1)c1cc(C(C)(C)C)cc3c4cc5c(cc4n2c13)c1c2ccccc2cc2c3c4ccccc4cc(N(c4ccccc4)c4ccccc4)c3n5c21 ; CC(C)(C)c1ccc2c(c1)c1cc(C(C)(C)C)cc3c4cc5c(cc4n2c13)c1c2ccccc2cc2c3c4ccccc4cc(-c4ccc6c(c4)c4ccccc4n6-c4ccccc4)c3n5c21 ; CC(C)(C)c1cc(N(c2ccccc2)c2ccccc2)c2c(c1)c1cc(C(C)(C)C)cc3c4cc5c(cc4n2c31)c1c2ccccc2cc2c3c4ccccc4ccc3n5c21]

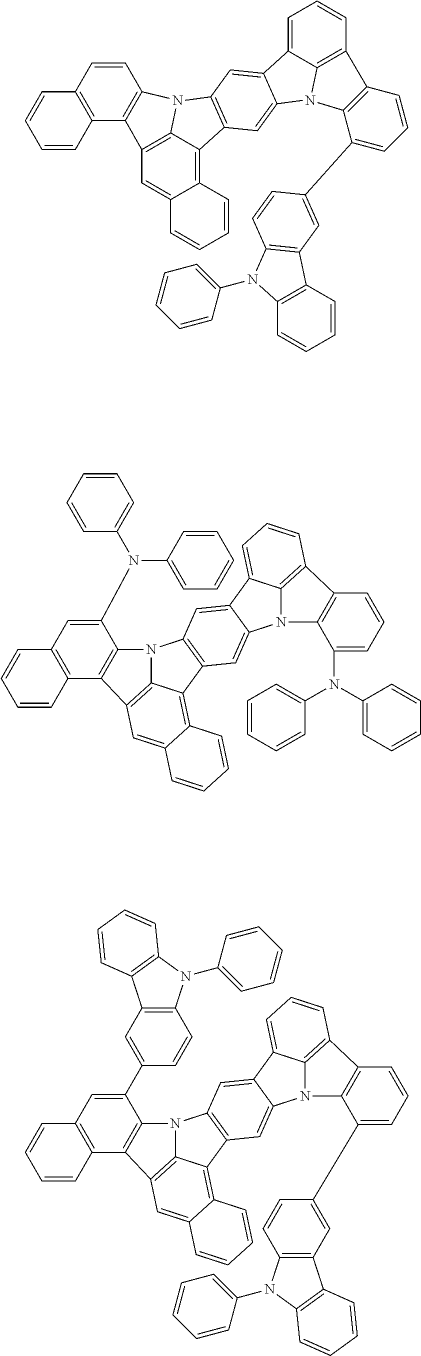 [SMILES: c1ccc(-n2c3ccccc3c3cc(-c4cccc5c6cccc7c8cc9c(cc8n(c45)c76)c4c5ccccc5cc5c6c7ccccc7ccc6n9c54)ccc32)cc1 ; c1ccc(N(c2ccccc2)c2cccc3c4cccc5c6cc7c(cc6n(c23)c54)c2c3ccccc3cc3c4c5ccccc5cc(N(c5ccccc5)c5ccccc5)c4n7c32)cc1 ; c1ccc(-n2c3ccccc3c3cc(-c4cccc5c6cccc7c8cc9c(cc8n(c45)c76)c4c5ccccc5cc5c6c7ccccc7cc(-c7ccc8c(c7)c7ccccc7n8-c7ccccc7)c6n9c54)ccc32)cc1]

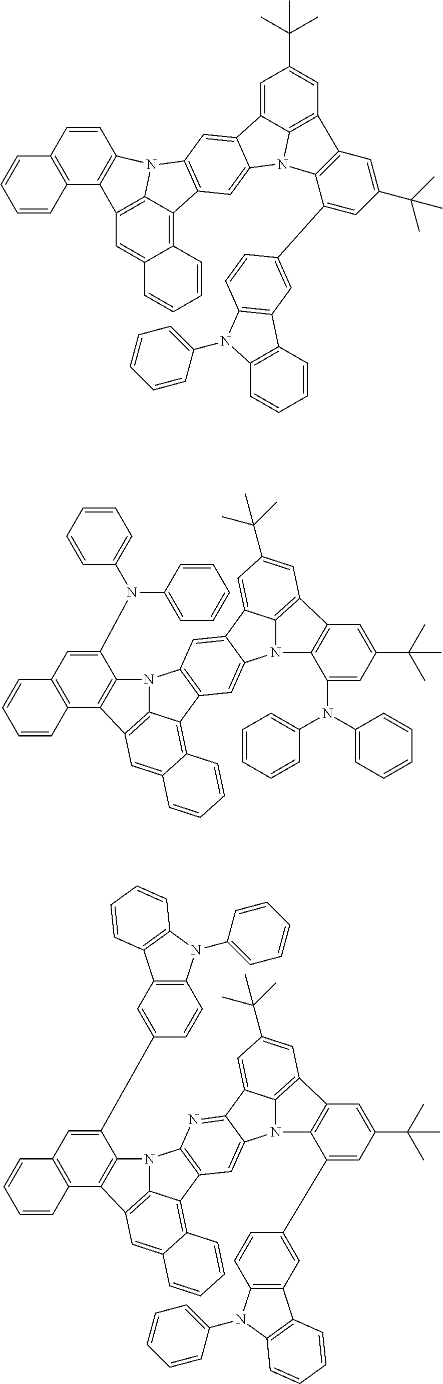 [SMILES: CC(C)(C)c1cc(-c2ccc3c(c2)c2ccccc2n3-c2ccccc2)c2c(c1)c1cc(C(C)(C)C)cc3c4cc5c(cc4n2c31)c1c2ccccc2cc2c3c4ccccc4ccc3n5c21 ; CC(C)(C)c1cc(N(c2ccccc2)c2ccccc2)c2c(c1)c1cc(C(C)(C)C)cc3c4cc5c(cc4n2c31)c1c2ccccc2cc2c3c4ccccc4cc(N(c4ccccc4)c4ccccc4)c3n5c21 ; CC(C)(C)c1cc(-c2ccc3c(c2)c2ccccc2n3-c2ccccc2)c2c(c1)c1cc(C(C)(C)C)cc3c4nc5c(cc4n2c13)c1c2ccccc2cc2c3c4ccccc4cc(-c4ccc6c(c4)c4ccccc4n6-c4ccccc4)c3n5c21]

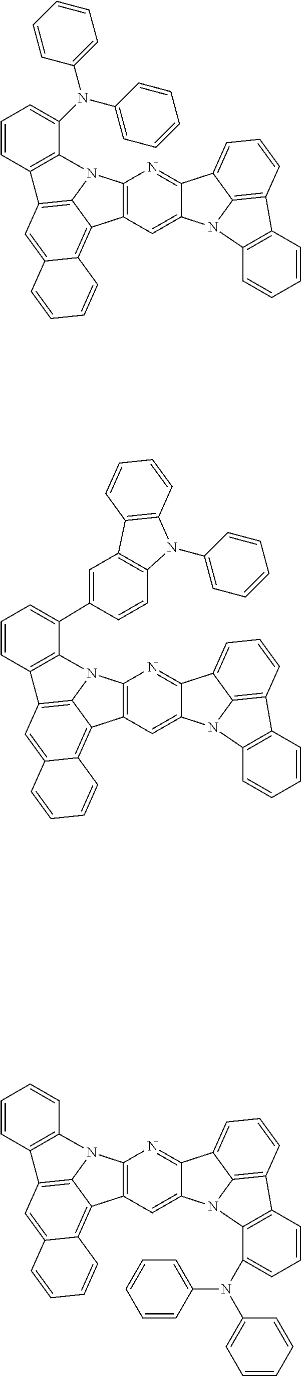 [SMILES: c1ccc(N(c2ccccc2)c2cccc3c4cc5ccccc5c5c6cc7c(nc6n(c23)c45)c2cccc3c4ccccc4n7c32)cc1 ; c1ccc(-n2c3ccccc3c3cc(-c4cccc5c6cc7ccccc7c7c8cc9c(nc8n(c45)c67)c4cccc5c6ccccc6n9c54)ccc32)cc1 ; c1ccc(N(c2ccccc2)c2cccc3c4cccc5c6nc7c(cc6n(c23)c45)c2c3ccccc3cc3c4ccccc4n7c32)cc1]

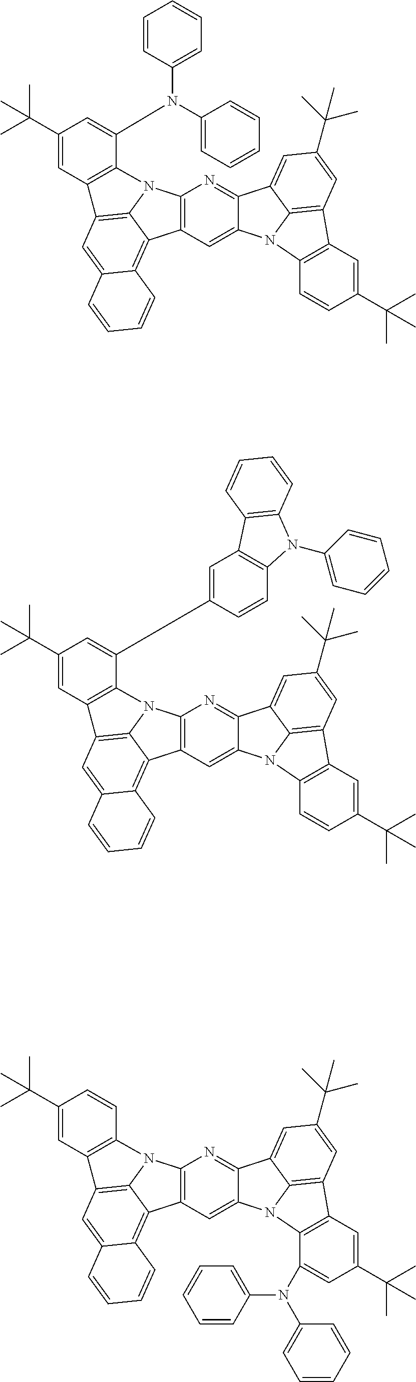

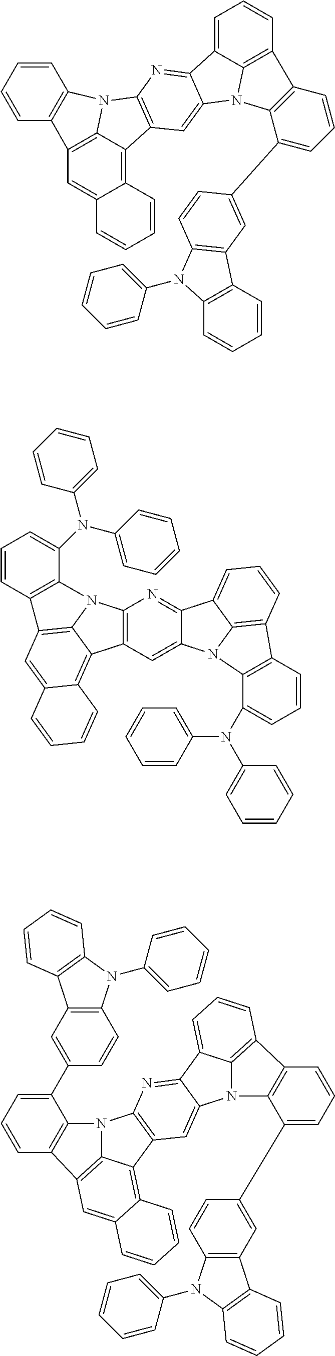 [SMILES: c1ccc(-n2c3ccccc3c3cc(-c4cccc5c6cccc7c8nc9c(cc8n(c45)c67)c4c5ccccc5cc5c6ccccc6n9c54)ccc32)cc1 ; c1ccc(N(c2ccccc2)c2cccc3c4cccc5c6nc7c(cc6n(c23)c45)c2c3ccccc3cc3c4cccc(N(c5ccccc5)c5ccccc5)c4n7c32)cc1 ; c1ccc(-n2c3ccccc3c3cc(-c4cccc5c6cccc7c8nc9c(cc8n(c45)c67)c4c5ccccc5cc5c6cccc(-c7ccc8c(c7)c7ccccc7n8-c7ccccc7)c6n9c54)ccc32)cc1]

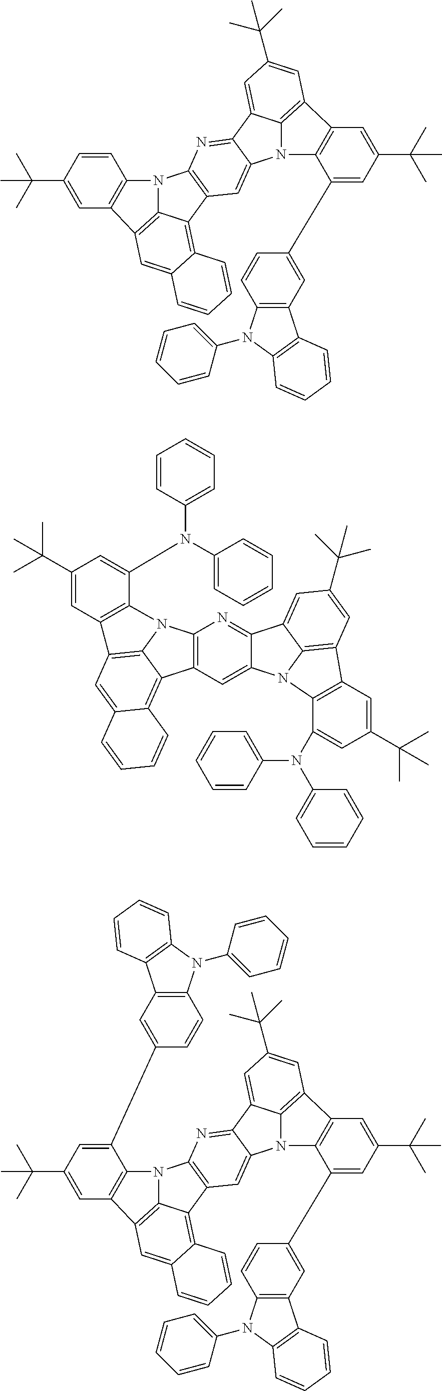

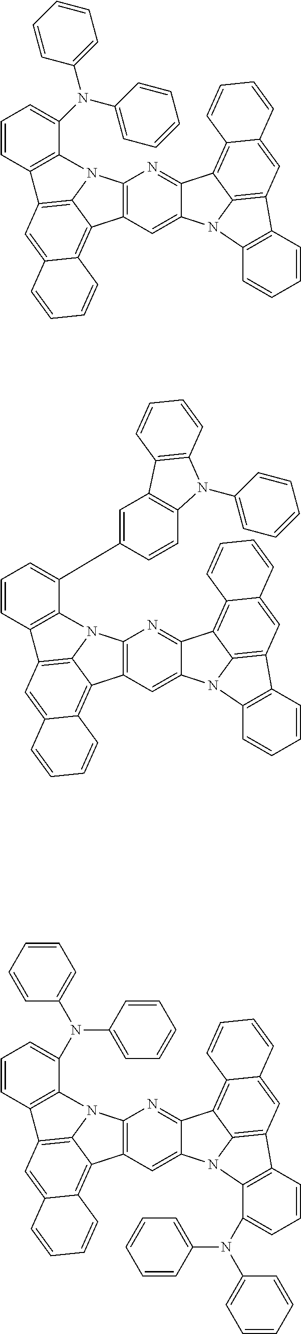 [SMILES: c1ccc(N(c2ccccc2)c2cccc3c4cc5ccccc5c5c6cc7c(nc6n(c23)c45)c2c3ccccc3cc3c4ccccc4n7c32)cc1 ; c1ccc(-n2c3ccccc3c3cc(-c4cccc5c6cc7ccccc7c7c8cc9c(nc8n(c45)c67)c4c5ccccc5cc5c6ccccc6n9c54)ccc32)cc1 ; c1ccc(N(c2ccccc2)c2cccc3c4cc5ccccc5c5c6nc7c(cc6n(c23)c45)c2c3ccccc3cc3c4cccc(N(c5ccccc5)c5ccccc5)c4n7c32)cc1]

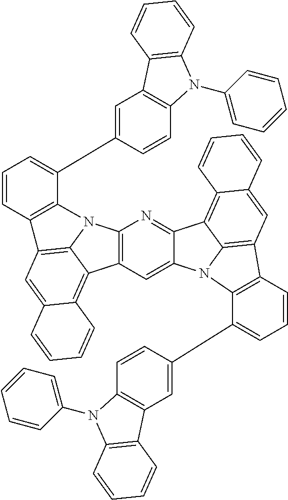 [SMILES: c1ccc(-n2c3ccccc3c3cc(-c4cccc5c6cc7ccccc7c7c8nc9c(cc8n(c45)c67)c4c5ccccc5cc5c6cccc(-c7ccc8c(c7)c7ccccc7n8-c7ccccc7)c6n9c54)ccc32)cc1]

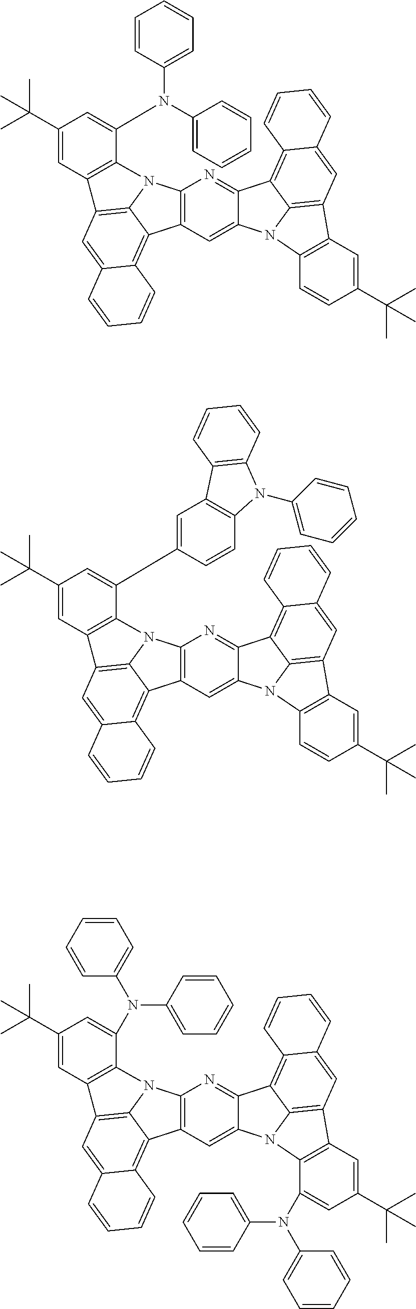

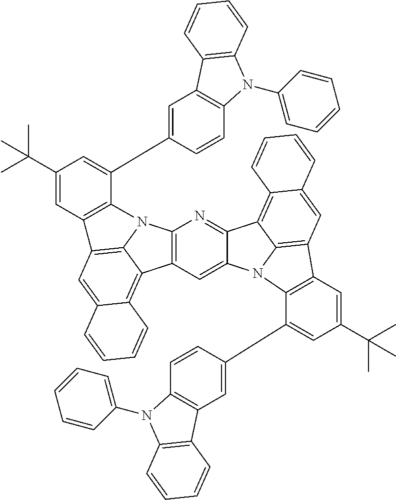 [SMILES: CC(C)(C)c1cc(-c2ccc3c(c2)c2ccccc2n3-c2ccccc2)c2c(c1)c1cc3ccccc3c3c4nc5c(cc4n2c13)c1c2ccccc2cc2c3cc(C(C)(C)C)cc(-c4ccc6c(c4)c4ccccc4n6-c4ccccc4)c3n5c21]

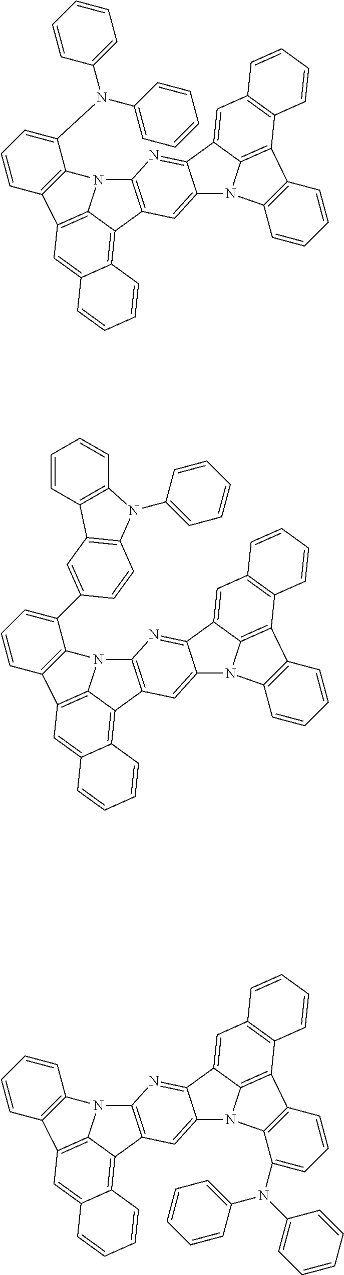 [SMILES: c1ccc(N(c2ccccc2)c2cccc3c4cc5ccccc5c5c6cc7c(nc6n(c23)c45)c2cc3ccccc3c3c4ccccc4n7c23)cc1 ; c1ccc(-n2c3ccccc3c3cc(-c4cccc5c6cc7ccccc7c7c8cc9c(nc8n(c45)c67)c4cc5ccccc5c5c6ccccc6n9c45)ccc32)cc1 ; c1ccc(N(c2ccccc2)c2cccc3c4c5ccccc5cc5c6nc7c(cc6n(c23)c54)c2c3ccccc3cc3c4ccccc4n7c32)cc1]

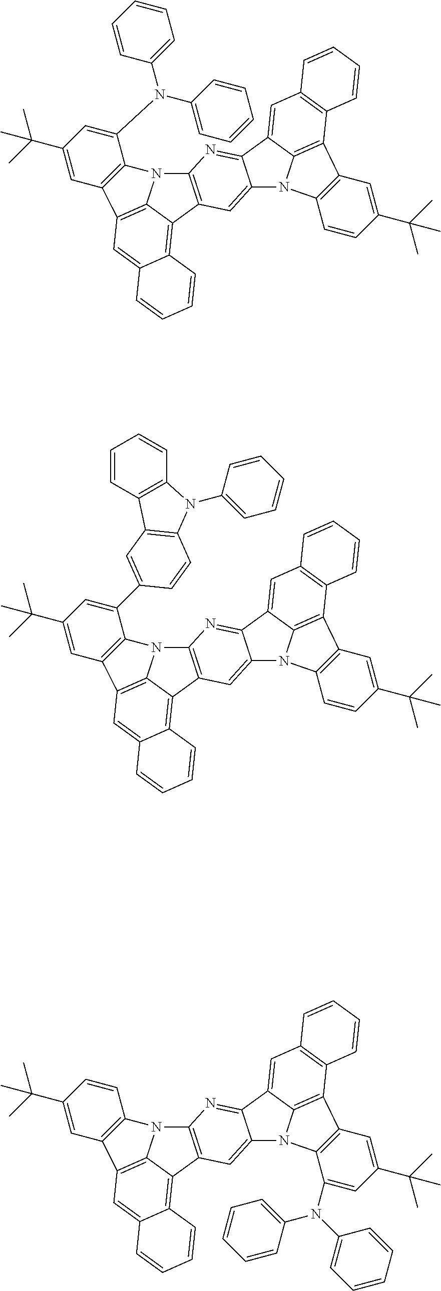

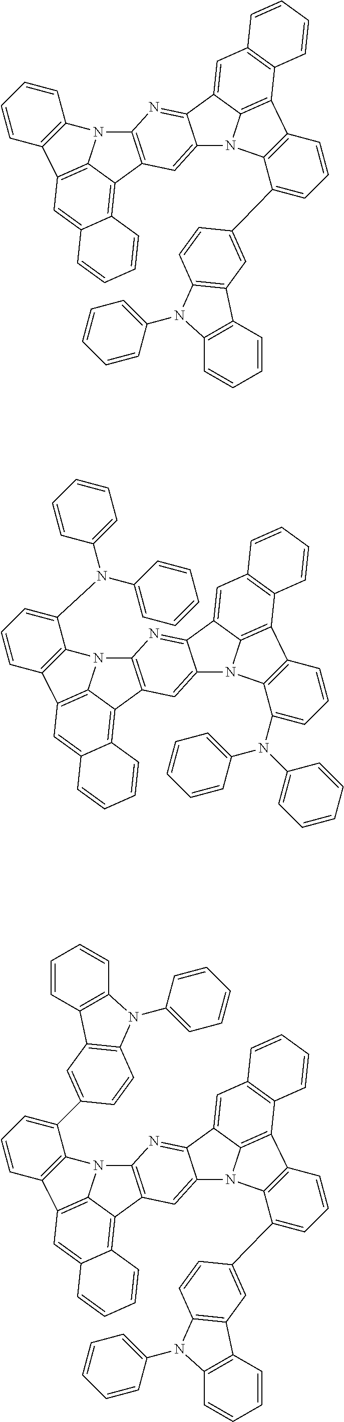 [SMILES: c1ccc(-n2c3ccccc3c3cc(-c4cccc5c6c7ccccc7cc7c8nc9c(cc8n(c45)c76)c4c5ccccc5cc5c6ccccc6n9c54)ccc32)cc1 ; c1ccc(N(c2ccccc2)c2cccc3c4c5ccccc5cc5c6nc7c(cc6n(c23)c54)c2c3ccccc3cc3c4cccc(N(c5ccccc5)c5ccccc5)c4n7c32)cc1 ; c1ccc(-n2c3ccccc3c3cc(-c4cccc5c6c7ccccc7cc7c8nc9c(cc8n(c45)c76)c4c5ccccc5cc5c6cccc(-c7ccc8c(c7)c7ccccc7n8-c7ccccc7)c6n9c54)ccc32)cc1]

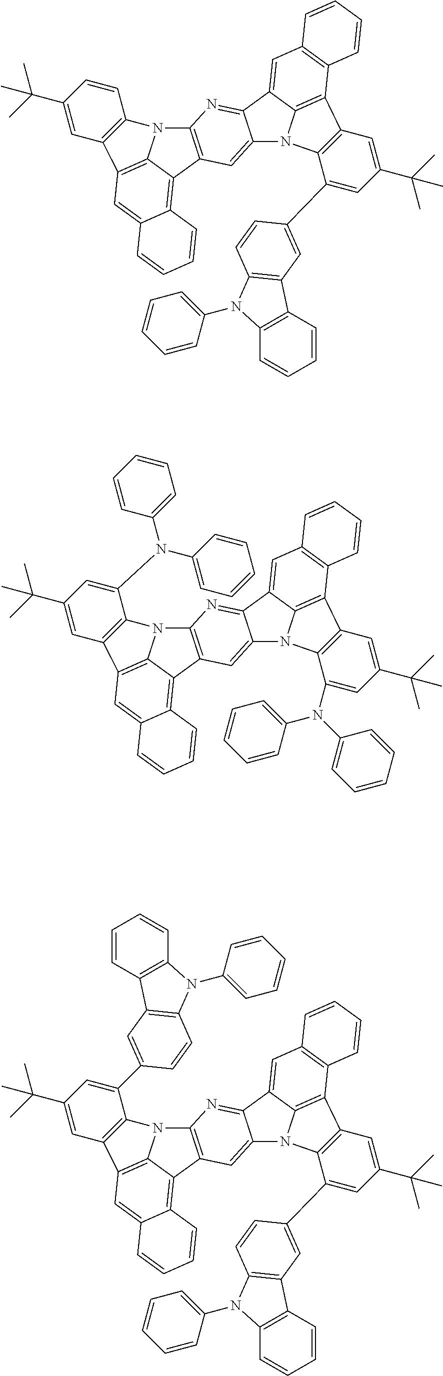

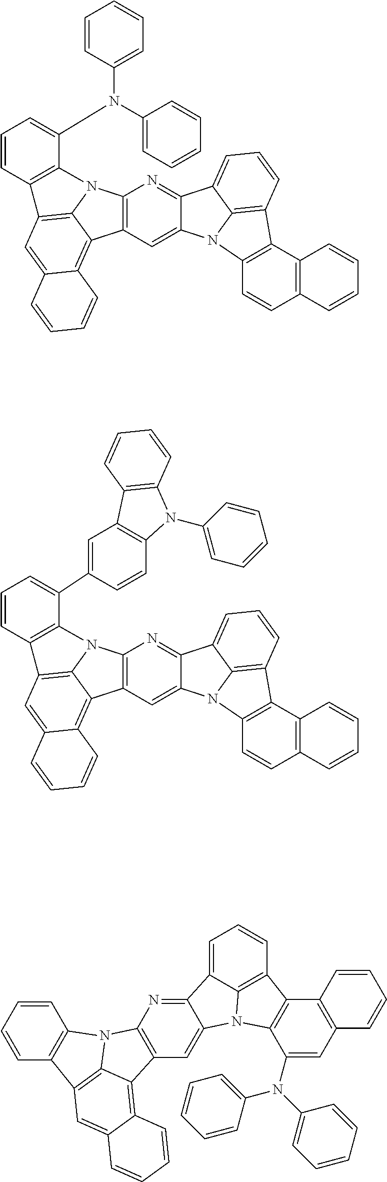 [SMILES: c1ccc(N(c2ccccc2)c2cccc3c4cc5ccccc5c5c6cc7c(nc6n(c23)c45)c2cccc3c4c5ccccc5ccc4n7c23)cc1 ; c1ccc(-n2c3ccccc3c3cc(-c4cccc5c6cc7ccccc7c7c8cc9c(nc8n(c45)c67)c4cccc5c6c7ccccc7ccc6n9c45)ccc32)cc1 ; c1ccc(N(c2ccccc2)c2cc3ccccc3c3c4cccc5c6nc7c(cc6n(c23)c54)c2c3ccccc3cc3c4ccccc4n7c32)cc1]

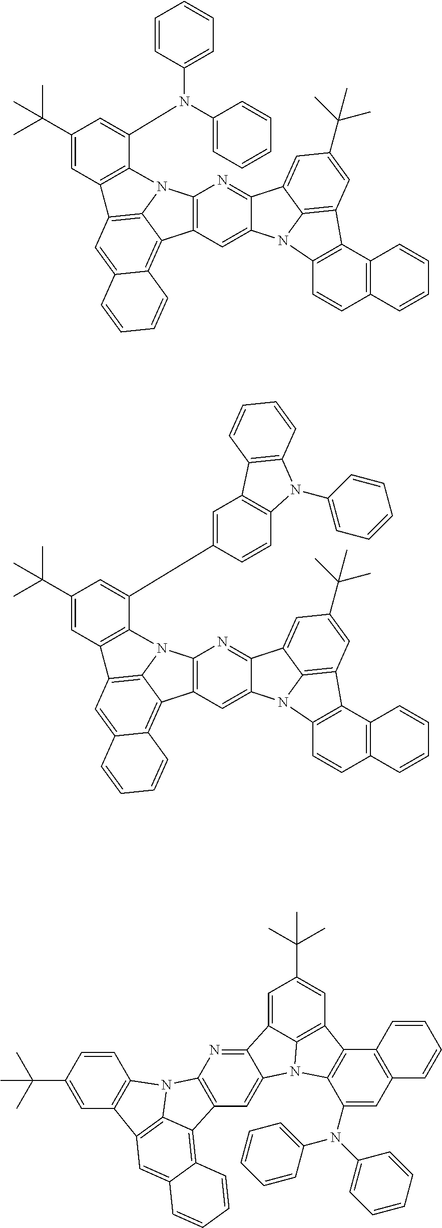 [SMILES: CC(C)(C)c1cc(N(c2ccccc2)c2ccccc2)c2c(c1)c1cc3ccccc3c3c4cc5c(nc4n2c13)c1cc(C(C)(C)C)cc2c3c4ccccc4ccc3n5c12 ; CC(C)(C)c1cc(-c2ccc3c(c2)c2ccccc2n3-c2ccccc2)c2c(c1)c1cc3ccccc3c3c4cc5c(nc4n2c13)c1cc(C(C)(C)C)cc2c3c4ccccc4ccc3n5c12 ; CC(C)(C)c1ccc2c(c1)c1cc3ccccc3c3c4cc5c(nc4n2c13)c1cc(C(C)(C)C)cc2c3c4ccccc4cc(N(c4ccccc4)c4ccccc4)c3n5c12]

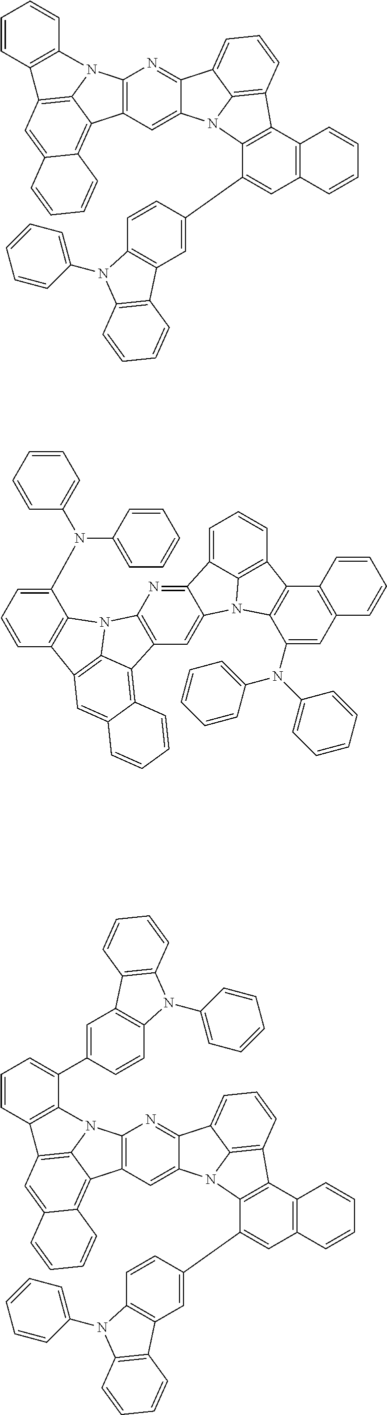 [SMILES: c1ccc(-n2c3ccccc3c3cc(-c4cc5ccccc5c5c6cccc7c8nc9c(cc8n(c45)c76)c4c5ccccc5cc5c6ccccc6n9c54)ccc32)cc1 ; c1ccc(N(c2ccccc2)c2cc3ccccc3c3c4cccc5c6nc7c(cc6n(c23)c54)c2c3ccccc3cc3c4cccc(N(c5ccccc5)c5ccccc5)c4n7c32)cc1 ; c1ccc(-n2c3ccccc3c3cc(-c4cc5ccccc5c5c6cccc7c8nc9c(cc8n(c45)c76)c4c5ccccc5cc5c6cccc(-c7ccc8c(c7)c7ccccc7n8-c7ccccc7)c6n9c54)ccc32)cc1]

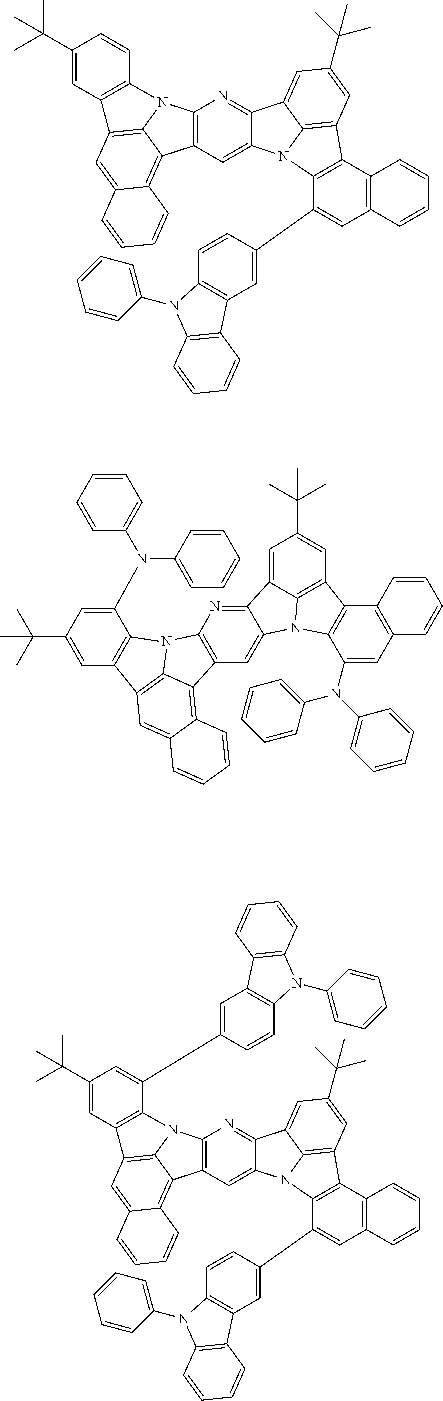 [SMILES: CC(C)(C)c1ccc2c(c1)c1cc3ccccc3c3c4cc5c(nc4n2c13)c1cc(C(C)(C)C)cc2c3c4ccccc4cc(-c4ccc6c(c4)c4ccccc4n6-c4ccccc4)c3n5c12 ; CC(C)(C)c1cc(N(c2ccccc2)c2ccccc2)c2c(c1)c1cc3ccccc3c3c4cc5c(nc4n2c13)c1cc(C(C)(C)C)cc2c3c4ccccc4cc(N(c4ccccc4)c4ccccc4)c3n5c12 ; CC(C)(C)c1cc(-c2ccc3c(c2)c2ccccc2n3-c2ccccc2)c2c(c1)c1cc3ccccc3c3c4cc5c(nc4n2c13)c1cc(C(C)(C)C)cc2c3c4ccccc4cc(-c4ccc6c(c4)c4ccccc4n6-c4ccccc4)c3n5c12]

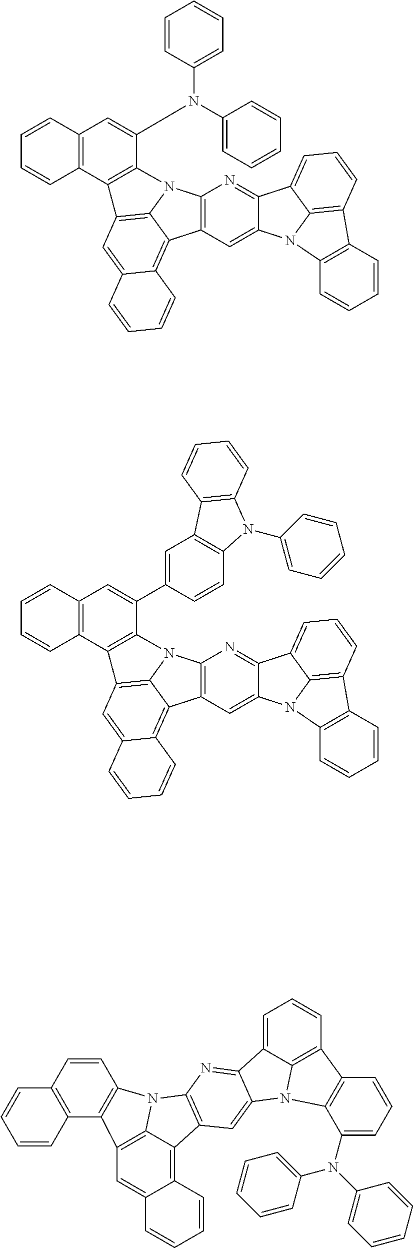 [SMILES: c1ccc(N(c2ccccc2)c2cc3ccccc3c3c4cc5ccccc5c5c6cc7c(nc6n(c23)c45)c2cccc3c4ccccc4n7c32)cc1 ; c1ccc(-n2c3ccccc3c3cc(-c4cc5ccccc5c5c6cc7ccccc7c7c8cc9c(nc8n(c45)c67)c4cccc5c6ccccc6n9c54)ccc32)cc1 ; c1ccc(N(c2ccccc2)c2cccc3c4cccc5c6nc7c(cc6n(c23)c45)c2c3ccccc3cc3c4c5ccccc5ccc4n7c32)cc1]

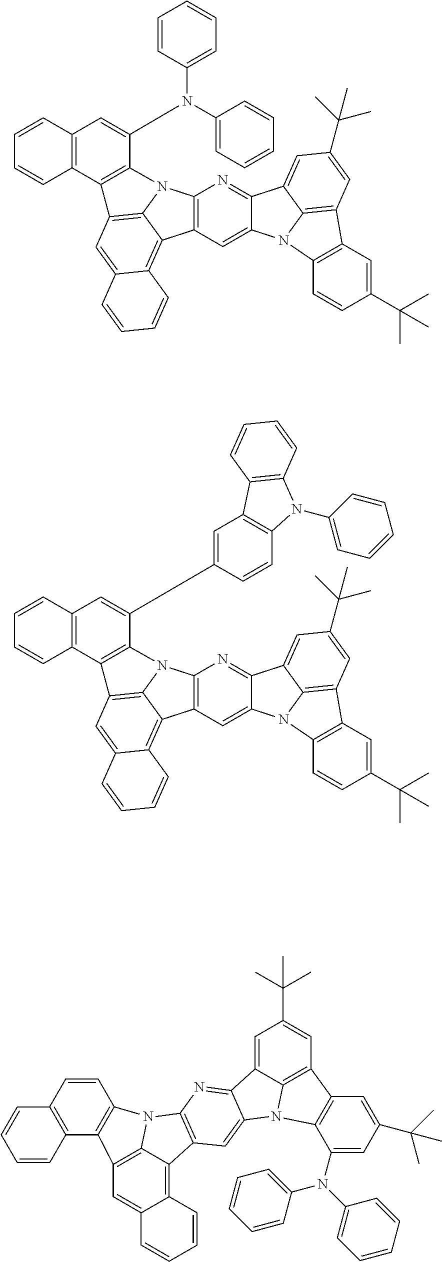 [SMILES: CC(C)(C)c1ccc2c(c1)c1cc(C(C)(C)C)cc3c4nc5c(cc4n2c13)c1c2ccccc2cc2c3c4ccccc4cc(N(c4ccccc4)c4ccccc4)c3n5c21 ; CC(C)(C)c1ccc2c(c1)c1cc(C(C)(C)C)cc3c4nc5c(cc4n2c13)c1c2ccccc2cc2c3c4ccccc4cc(-c4ccc6c(c4)c4ccccc4n6-c4ccccc4)c3n5c21 ; CC(C)(C)c1cc(N(c2ccccc2)c2ccccc2)c2c(c1)c1cc(C(C)(C)C)cc3c4nc5c(cc4n2c13)c1c2ccccc2cc2c3c4ccccc4ccc3n5c21]

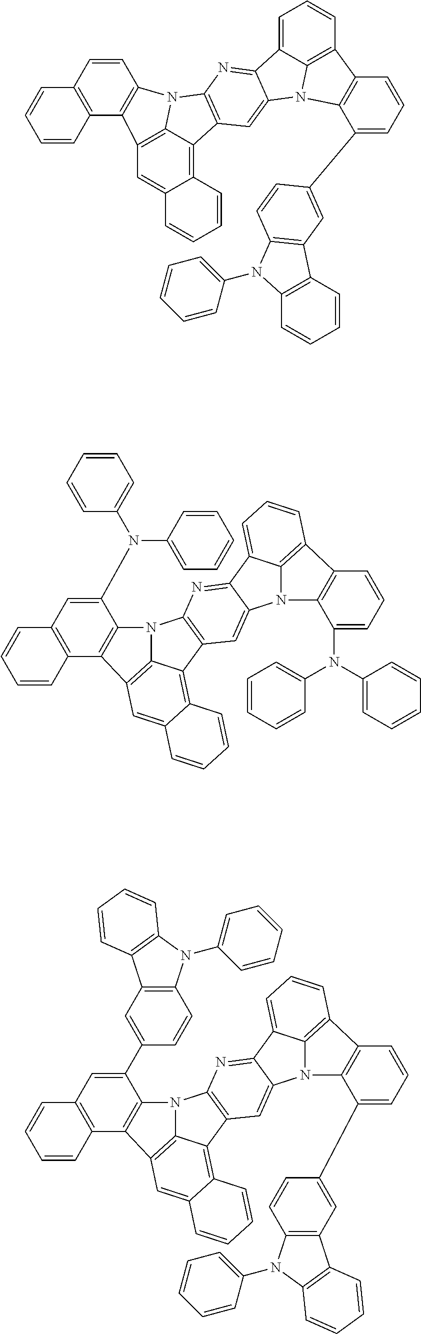 [SMILES: c1ccc(-n2c3ccccc3c3cc(-c4cccc5c6cccc7c8nc9c(cc8n(c45)c67)c4c5ccccc5cc5c6c7ccccc7ccc6n9c54)ccc32)cc1 ; c1ccc(N(c2ccccc2)c2cccc3c4cccc5c6nc7c(cc6n(c23)c45)c2c3ccccc3cc3c4c5ccccc5cc(N(c5ccccc5)c5ccccc5)c4n7c32)cc1 ; c1ccc(-n2c3ccccc3c3cc(-c4cccc5c6cccc7c8nc9c(cc8n(c45)c67)c4c5ccccc5cc5c6c7ccccc7cc(-c7ccc8c(c7)c7ccccc7n8-c7ccccc7)c6n9c54)ccc32)cc1]

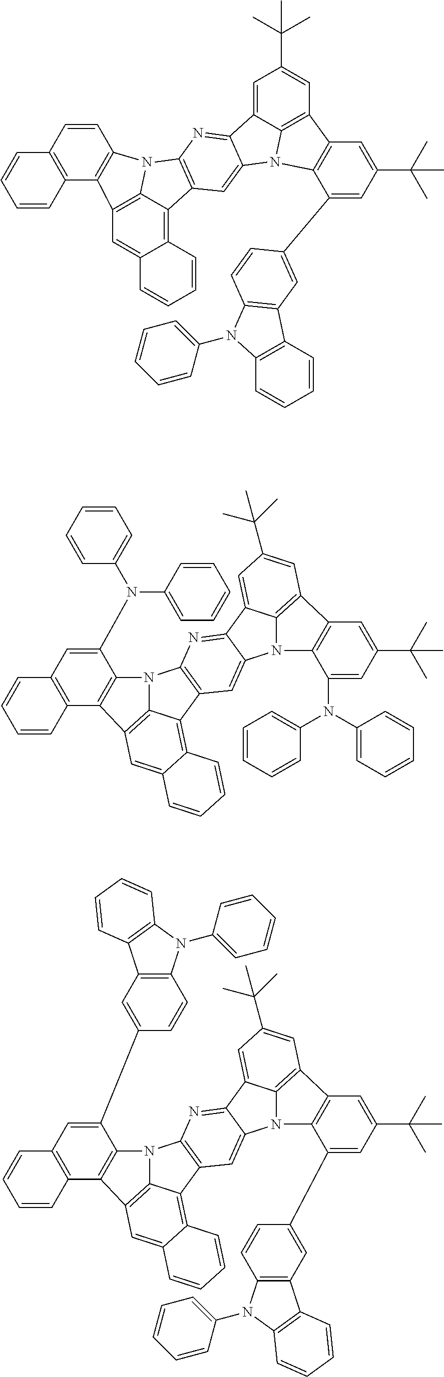 [SMILES: CC(C)(C)c1cc(-c2ccc3c(c2)c2ccccc2n3-c2ccccc2)c2c(c1)c1cc(C(C)(C)C)cc3c4nc5c(cc4n2c13)c1c2ccccc2cc2c3c4ccccc4ccc3n5c21 ; CC(C)(C)c1cc(N(c2ccccc2)c2ccccc2)c2c(c1)c1cc(C(C)(C)C)cc3c4nc5c(cc4n2c13)c1c2ccccc2cc2c3c4ccccc4cc(N(c4ccccc4)c4ccccc4)c3n5c21 ; CC(C)(C)c1cc(-c2ccc3c(c2)c2ccccc2n3-c2ccccc2)c2c(c1)c1cc(C(C)(C)C)cc3c4nc5c(cc4n2c13)c1c2ccccc2cc2c3c4ccccc4cc(-c4ccc6c(c4)c4ccccc4n6-c4ccccc4)c3n5c21]

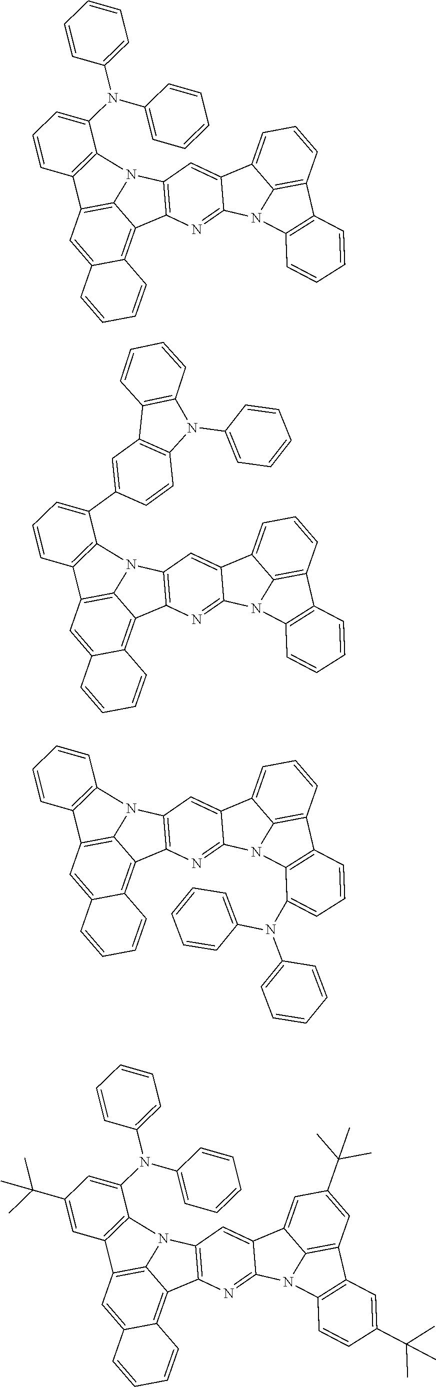

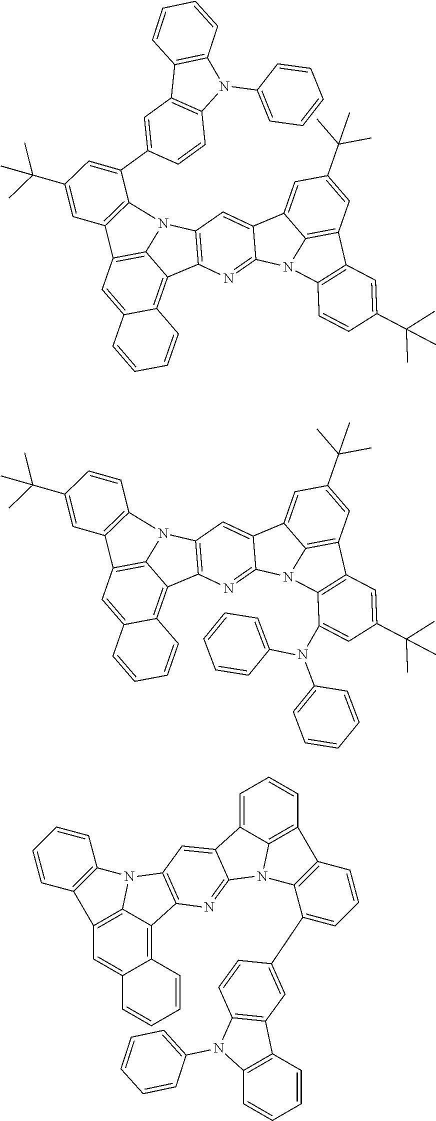

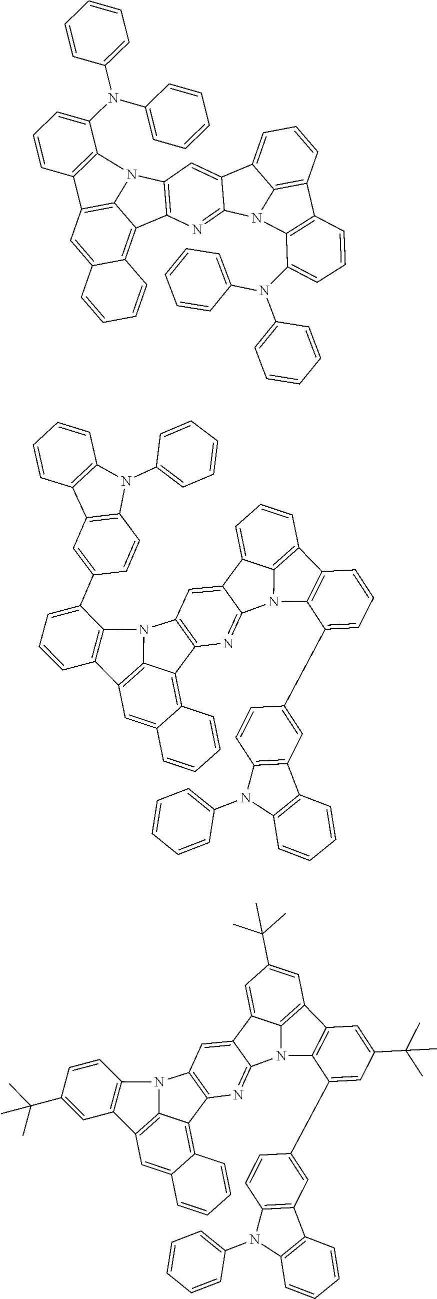 [SMILES: c1ccc(N(c2ccccc2)c2cccc3c4cc5ccccc5c5c6nc7c(cc6n(c23)c45)c2cccc3c4cccc(N(c5ccccc5)c5ccccc5)c4n7c23)cc1 ; c1ccc(-n2c3ccccc3c3cc(-c4cccc5c6cc7ccccc7c7c8nc9c(cc8n(c45)c67)c4cccc5c6cccc(-c7ccc8c(c7)c7ccccc7n8-c7ccccc7)c6n9c45)ccc32)cc1 ; CC(C)(C)c1ccc2c(c1)c1cc3ccccc3c3c4nc5c(cc4n2c13)c1cc(C(C)(C)C)cc2c3cc(C(C)(C)C)cc(-c4ccc6c(c4)c4ccccc4n6-c4ccccc4)c3n5c12]

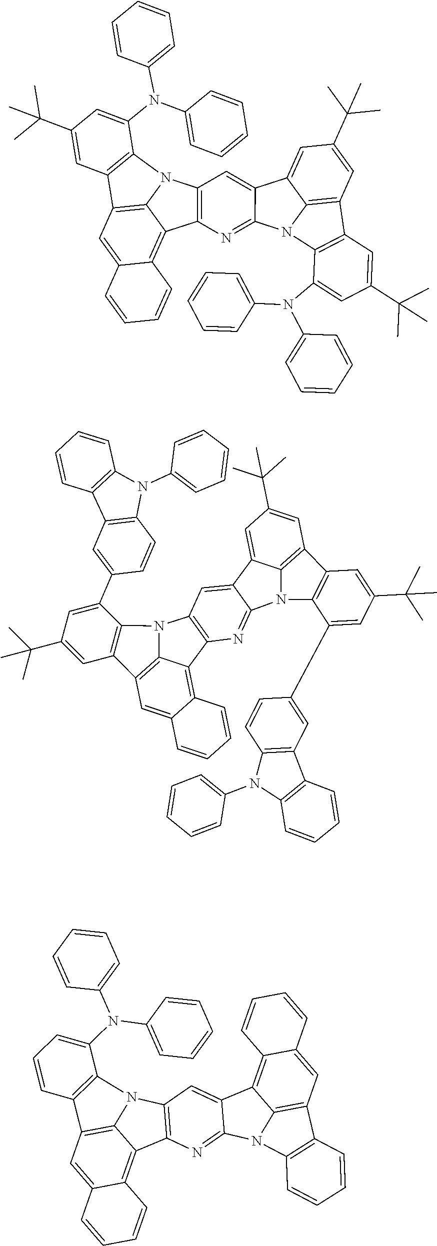

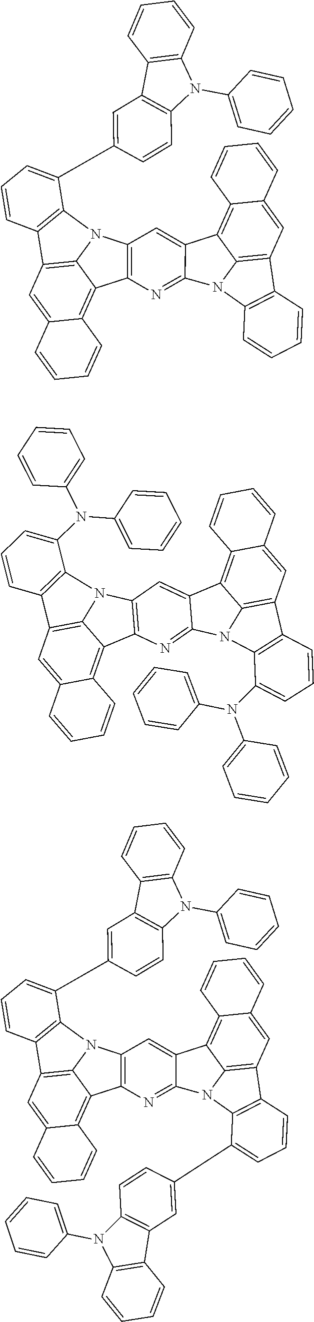 [SMILES: c1ccc(-n2c3ccccc3c3cc(-c4cccc5c6cc7ccccc7c7c8nc9c(cc8n(c45)c67)c4c5ccccc5cc5c6ccccc6n9c54)ccc32)cc1 ; c1ccc(N(c2ccccc2)c2cccc3c4cc5ccccc5c5c6nc7c(cc6n(c23)c45)c2c3ccccc3cc3c4cccc(N(c5ccccc5)c5ccccc5)c4n7c32)cc1 ; c1ccc(-n2c3ccccc3c3cc(-c4cccc5c6cc7ccccc7c7c8nc9c(cc8n(c45)c67)c4c5ccccc5cc5c6cccc(-c7ccc8c(c7)c7ccccc7n8-c7ccccc7)c6n9c54)ccc32)cc1]

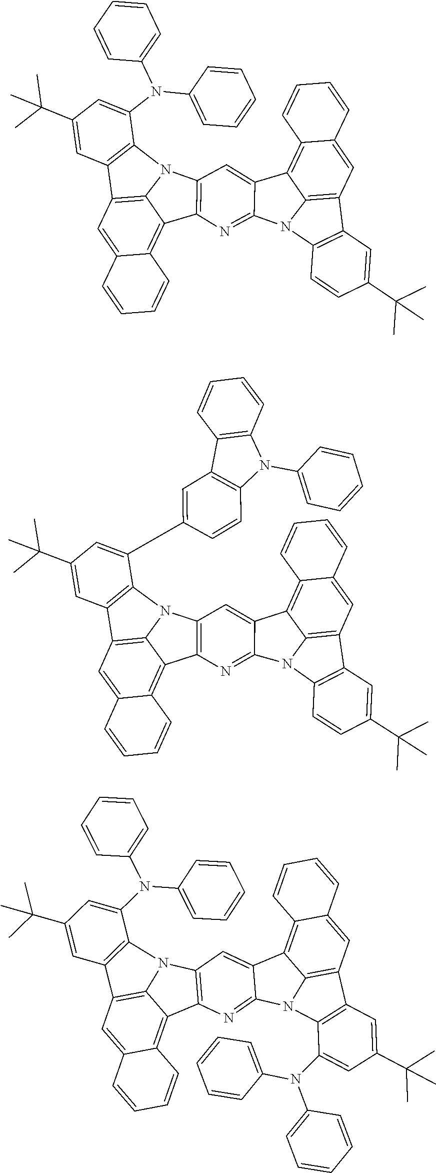

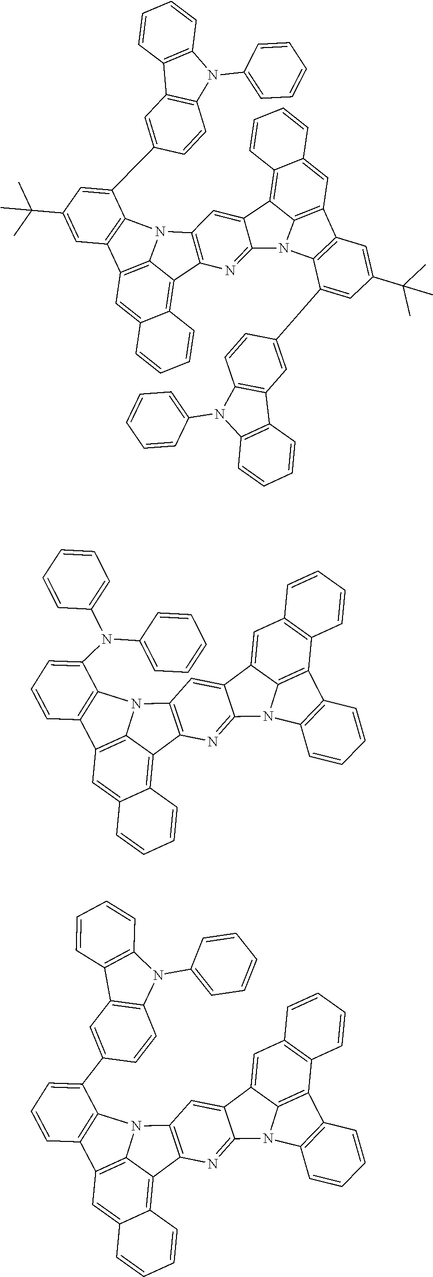 [SMILES: CC(C)(C)c1cc(-c2ccc3c(c2)c2ccccc2n3-c2ccccc2)c2c(c1)c1cc3ccccc3c3c4nc5c(cc4n2c13)c1c2ccccc2cc2c3cc(C(C)(C)C)cc(-c4ccc6c(c4)c4ccccc4n6-c4ccccc4)c3n5c21 ; c1ccc(N(c2ccccc2)c2cccc3c4cc5ccccc5c5c6nc7c(cc6n(c23)c45)c2cc3ccccc3c3c4ccccc4n7c23)cc1 ; c1ccc(-n2c3ccccc3c3cc(-c4cccc5c6cc7ccccc7c7c8nc9c(cc8n(c45)c67)c4cc5ccccc5c5c6ccccc6n9c45)ccc32)cc1]

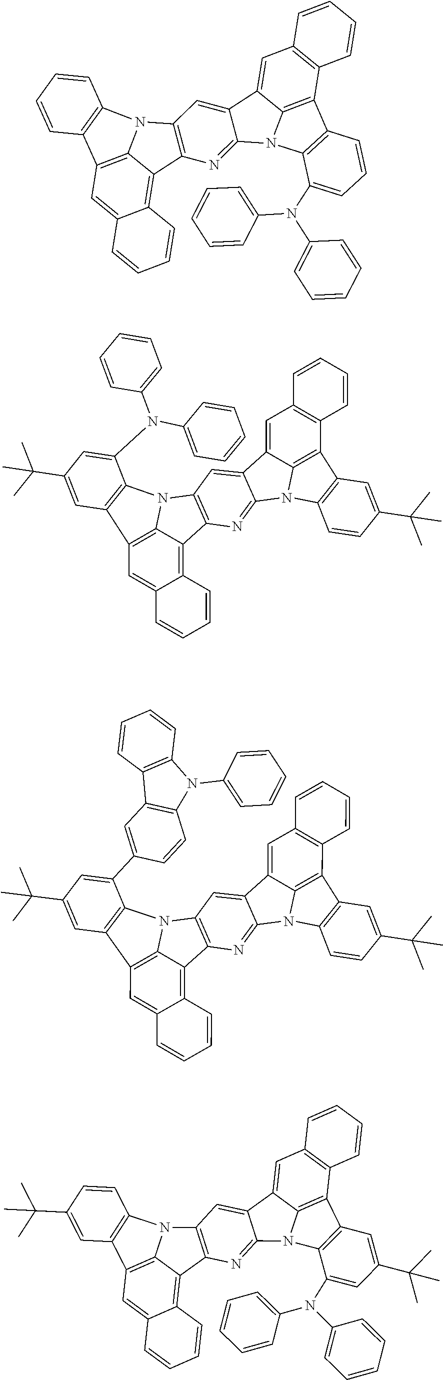

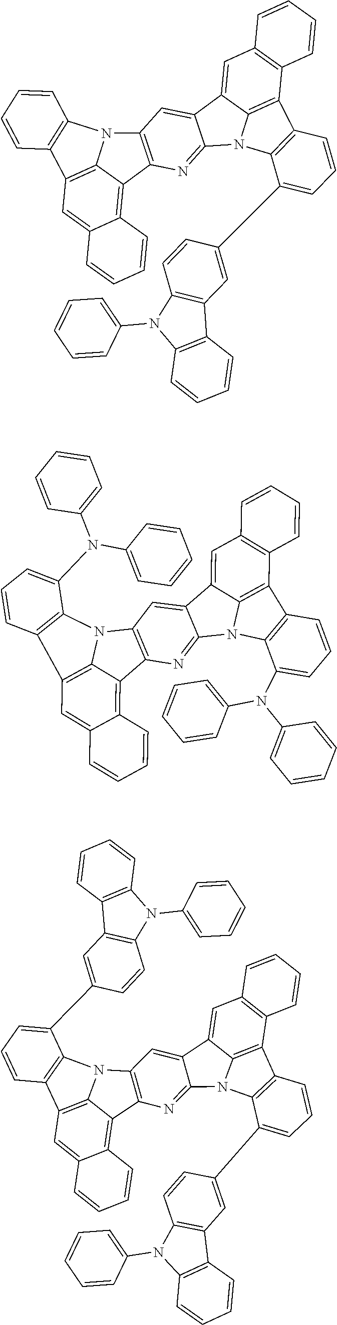 [SMILES: c1ccc(-n2c3ccccc3c3cc(-c4cccc5c6c7ccccc7cc7c8cc9c(nc8n(c45)c76)c4c5ccccc5cc5c6ccccc6n9c54)ccc32)cc1 ; c1ccc(N(c2ccccc2)c2cccc3c4cc5ccccc5c5c6nc7c(cc6n(c23)c45)c2cc3ccccc3c3c4cccc(N(c5ccccc5)c5ccccc5)c4n7c23)cc1 ; c1ccc(-n2c3ccccc3c3cc(-c4cccc5c6cc7ccccc7c7c8nc9c(cc8n(c45)c67)c4cc5ccccc5c5c6cccc(-c7ccc8c(c7)c7ccccc7n8-c7ccccc7)c6n9c45)ccc32)cc1]

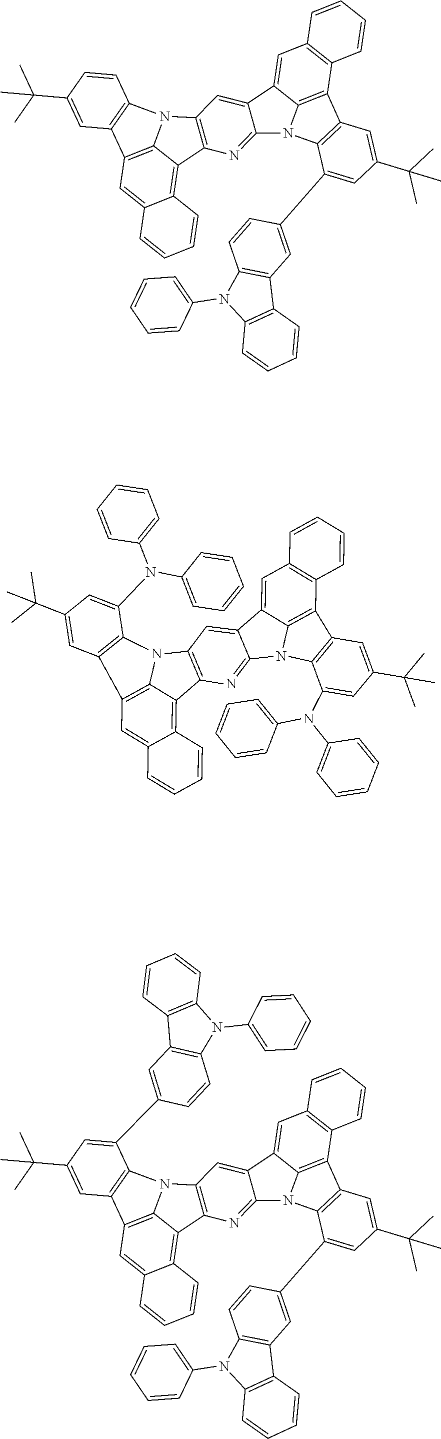

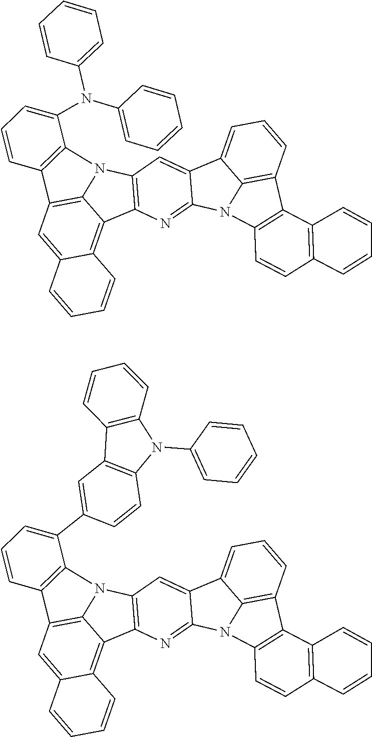 [SMILES: c1ccc(N(c2ccccc2)c2cccc3c4cc5ccccc5c5c6nc7c(cc6n(c23)c45)c2cccc3c4c5ccccc5ccc4n7c23)cc1 ; c1ccc(-n2c3ccccc3c3cc(-c4cccc5c6cc7ccccc7c7c8nc9c(cc8n(c45)c67)c4cccc5c6c7ccccc7ccc6n9c45)ccc32)cc1]

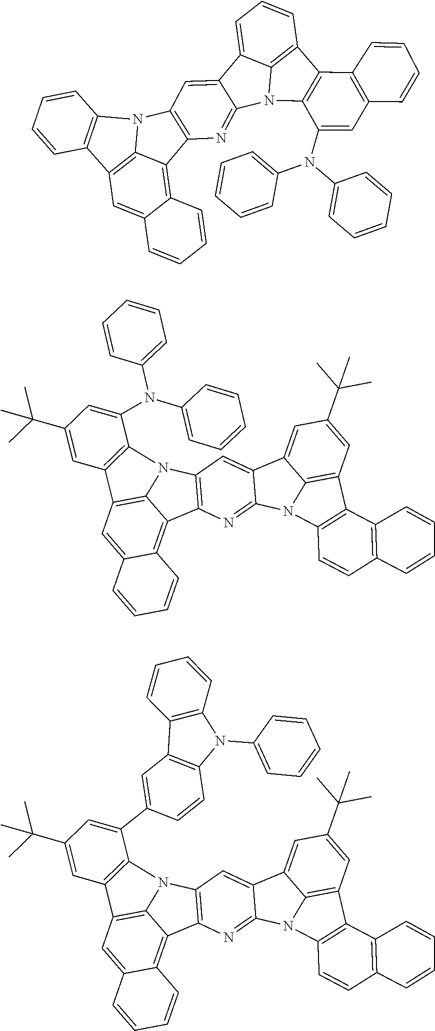 [SMILES: c1ccc(N(c2ccccc2)c2cc3ccccc3c3c4cccc5c6cc7c(nc6n(c23)c54)c2c3ccccc3cc3c4ccccc4n7c32)cc1 ; CC(C)(C)c1cc(N(c2ccccc2)c2ccccc2)c2c(c1)c1cc3ccccc3c3c4nc5c(cc4n2c13)c1cc(C(C)(C)C)cc2c3c4ccccc4ccc3n5c12 ; CC(C)(C)c1cc(-c2ccc3c(c2)c2ccccc2n3-c2ccccc2)c2c(c1)c1cc3ccccc3c3c4nc5c(cc4n2c13)c1cc(C(C)(C)C)cc2c3c4ccccc4ccc3n5c12]

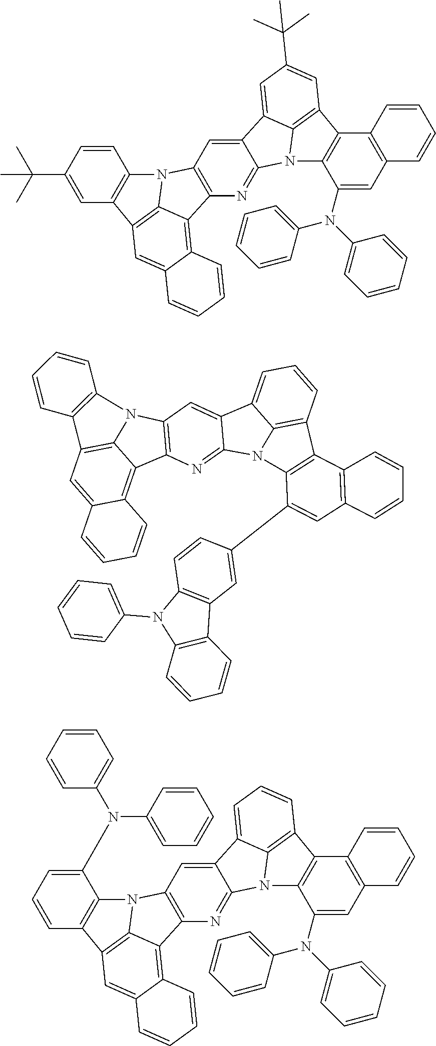 [SMILES: CC(C)(C)c1ccc2c(c1)c1cc3ccccc3c3c4nc5c(cc4n2c13)c1cc(C(C)(C)C)cc2c3c4ccccc4cc(N(c4ccccc4)c4ccccc4)c3n5c12 ; c1ccc(-n2c3ccccc3c3cc(-c4cc5ccccc5c5c6cccc7c8cc9c(nc8n(c45)c76)c4c5ccccc5cc5c6ccccc6n9c54)ccc32)cc1 ; c1ccc(N(c2ccccc2)c2cccc3c4cc5ccccc5c5c6nc7c(cc6n(c23)c45)c2cccc3c4c5ccccc5cc(N(c5ccccc5)c5ccccc5)c4n7c23)cc1]

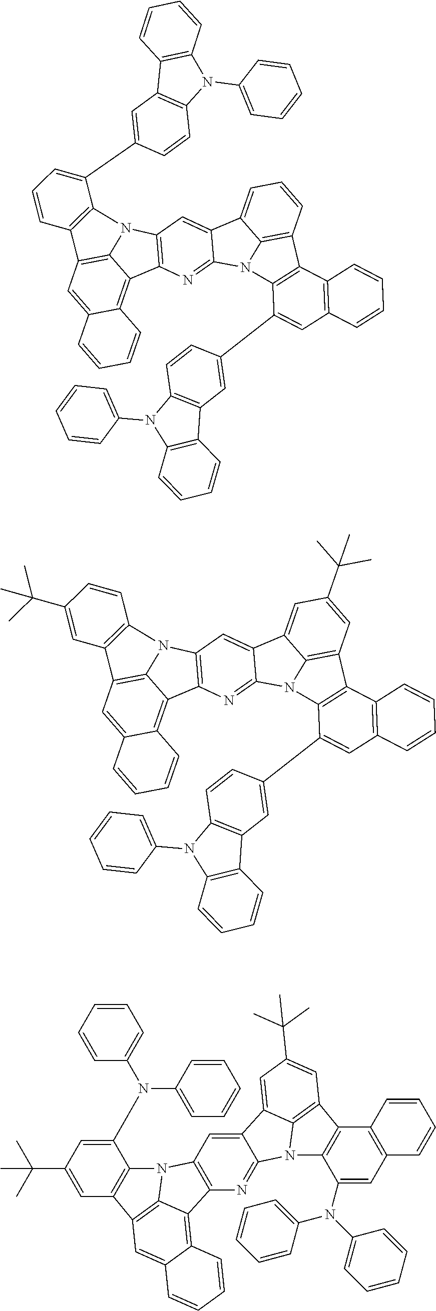 [SMILES: c1ccc(-n2c3ccccc3c3cc(-c4cccc5c6cc7ccccc7c7c8nc9c(cc8n(c45)c67)c4cccc5c6c7ccccc7cc(-c7ccc8c(c7)c7ccccc7n8-c7ccccc7)c6n9c45)ccc32)cc1 ; CC(C)(C)c1ccc2c(c1)c1cc3ccccc3c3c4nc5c(cc4n2c13)c1cc(C(C)(C)C)cc2c3c4ccccc4cc(-c4ccc6c(c4)c4ccccc4n6-c4ccccc4)c3n5c12 ; CC(C)(C)c1cc(N(c2ccccc2)c2ccccc2)c2c(c1)c1cc3ccccc3c3c4nc5c(cc4n2c13)c1cc(C(C)(C)C)cc2c3c4ccccc4cc(N(c4ccccc4)c4ccccc4)c3n5c12]

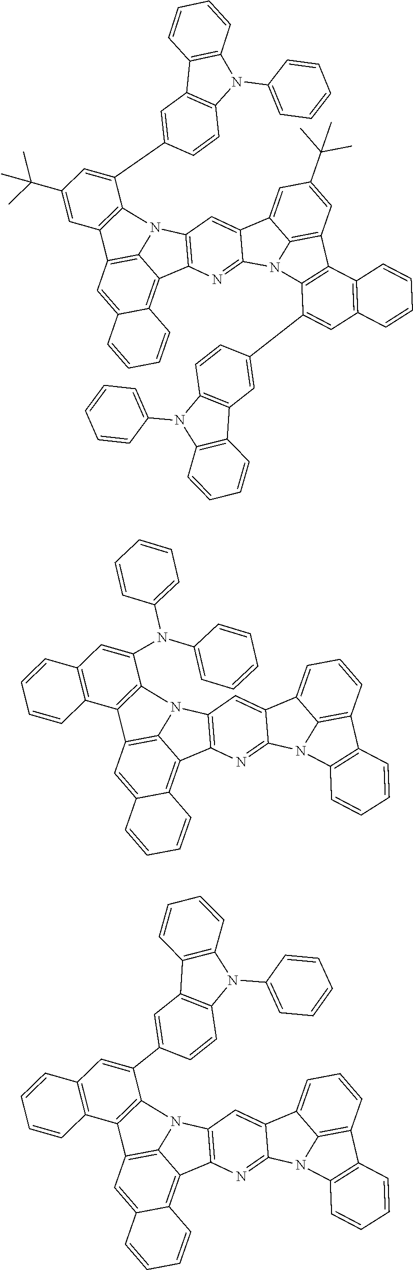 [SMILES: CC(C)(C)c1cc(-c2ccc3c(c2)c2ccccc2n3-c2ccccc2)c2c(c1)c1cc3ccccc3c3c4nc5c(cc4n2c13)c1cc(C(C)(C)C)cc2c3c4ccccc4cc(-c4ccc6c(c4)c4ccccc4n6-c4ccccc4)c3n5c12 ; c1ccc(N(c2ccccc2)c2cc3ccccc3c3c4cc5ccccc5c5c6nc7c(cc6n(c23)c45)c2cccc3c4ccccc4n7c32)cc1 ; c1ccc(-n2c3ccccc3c3cc(-c4cc5ccccc5c5c6cc7ccccc7c7c8nc9c(cc8n(c45)c67)c4cccc5c6ccccc6n9c54)ccc32)cc1]

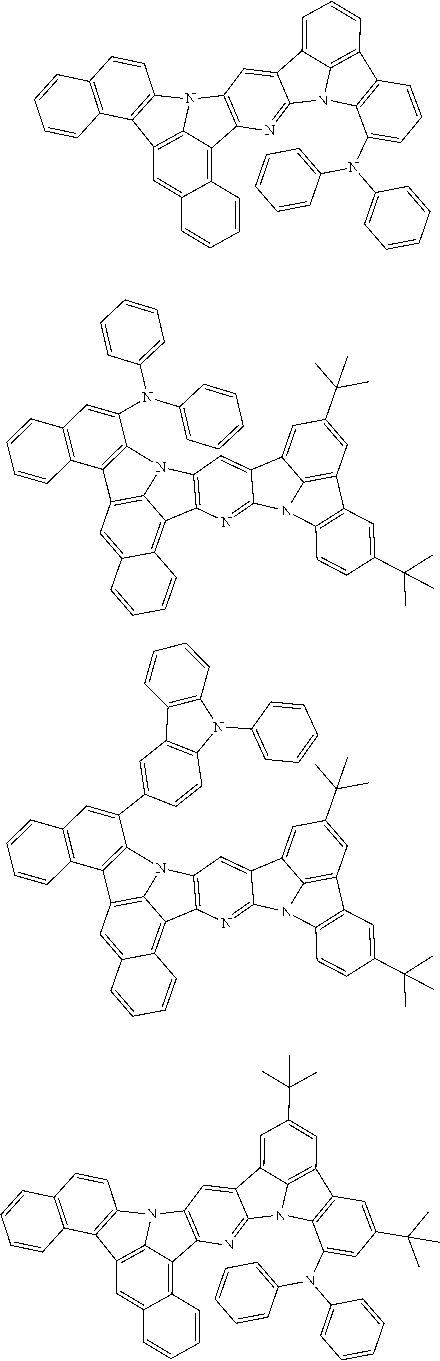

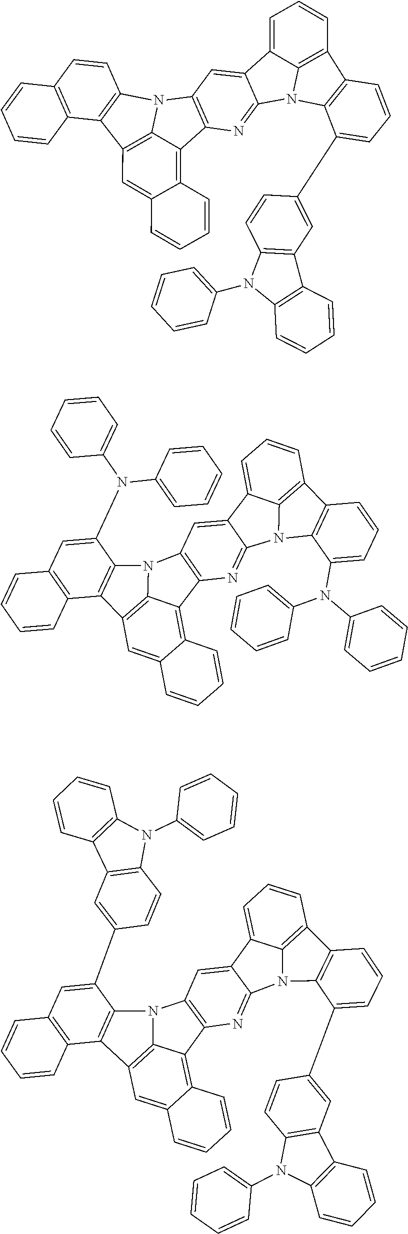 [SMILES: c1ccc(-n2c3ccccc3c3cc(-c4cccc5c6cccc7c8cc9c(nc8n(c45)c76)c4c5ccccc5cc5c6c7ccccc7ccc6n9c54)ccc32)cc1 ; c1ccc(N(c2ccccc2)c2cc3ccccc3c3c4cc5ccccc5c5c6nc7c(cc6n(c23)c45)c2cccc3c4cccc(N(c5ccccc5)c5ccccc5)c4n7c23)cc1 ; c1ccc(-n2c3ccccc3c3cc(-c4cc5ccccc5c5c6cc7ccccc7c7c8nc9c(cc8n(c45)c67)c4cccc5c6cccc(-c7ccc8c(c7)c7ccccc7n8-c7ccccc7)c6n9c45)ccc32)cc1]

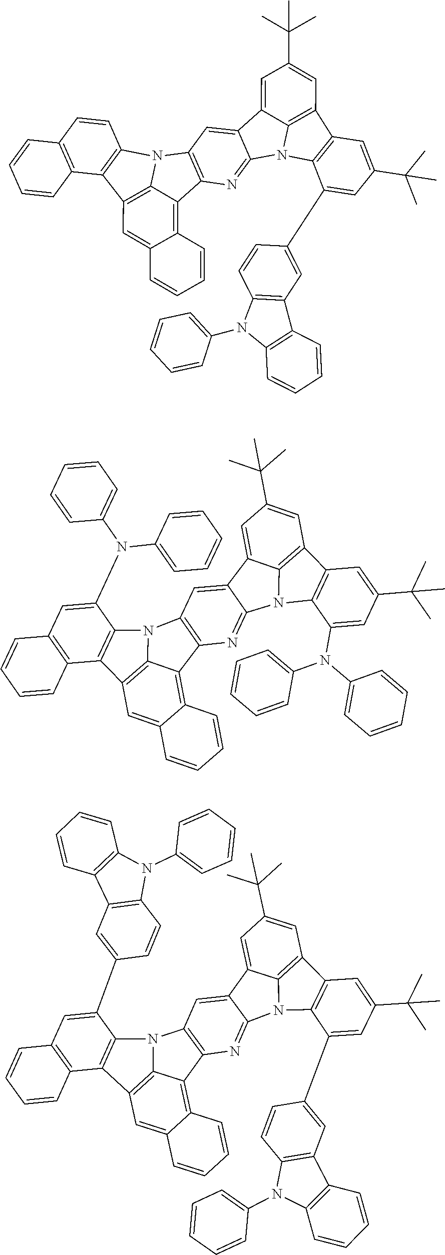

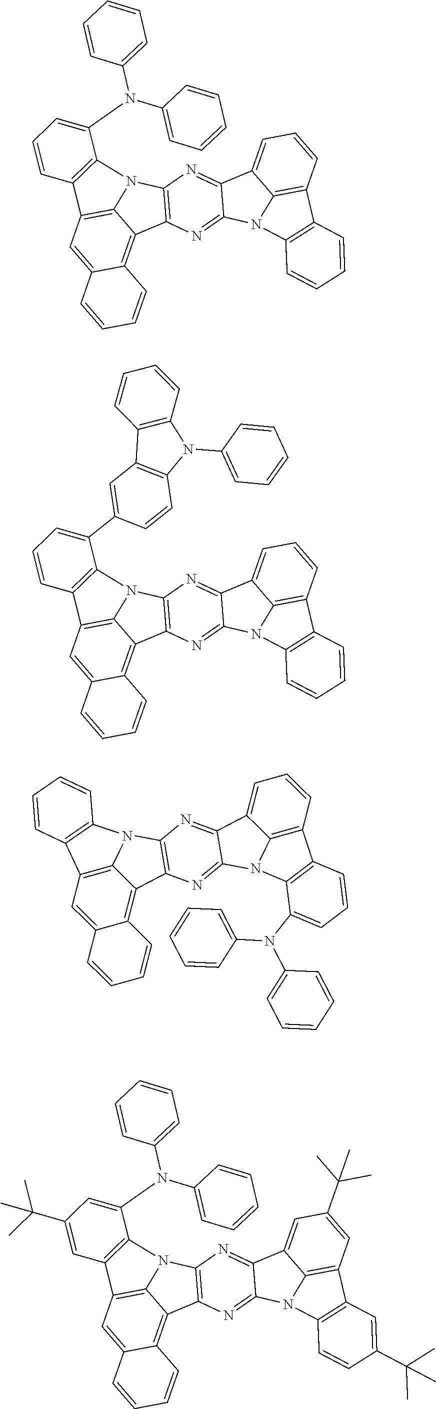 [SMILES: c1ccc(N(c2ccccc2)c2cccc3c4cc5ccccc5c5c6nc7c(nc6n(c23)c45)c2cccc3c4ccccc4n7c32)cc1 ; c1ccc(-n2c3ccccc3c3cc(-c4cccc5c6cc7ccccc7c7c8nc9c(nc8n(c45)c67)c4cccc5c6ccccc6n9c54)ccc32)cc1 ; c1ccc(N(c2ccccc2)c2cccc3c4cccc5c6nc7c(nc6n(c23)c45)c2c3ccccc3cc3c4ccccc4n7c32)cc1 ; CC(C)(C)c1ccc2c(c1)c1cc(C(C)(C)C)cc3c4nc5c(nc4n2c13)c1c2ccccc2cc2c3cc(C(C)(C)C)cc(N(c4ccccc4)c4ccccc4)c3n5c21]

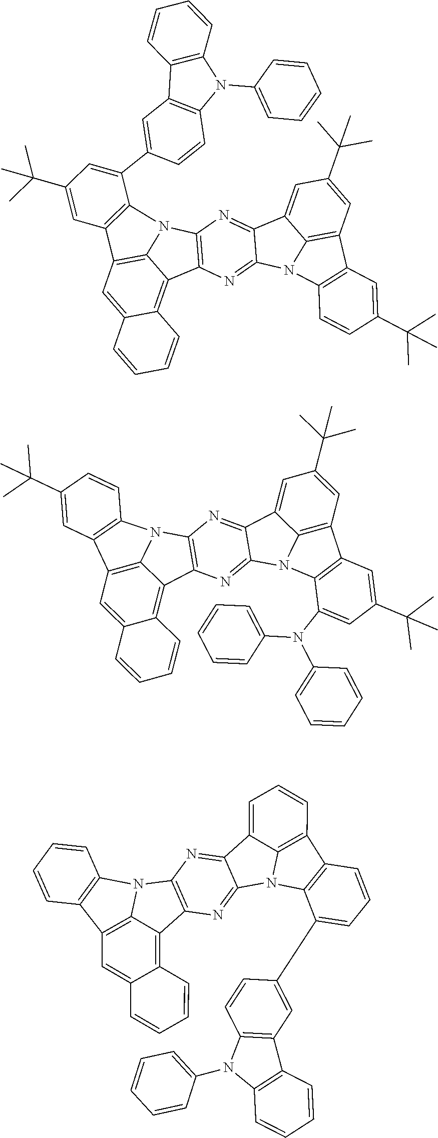

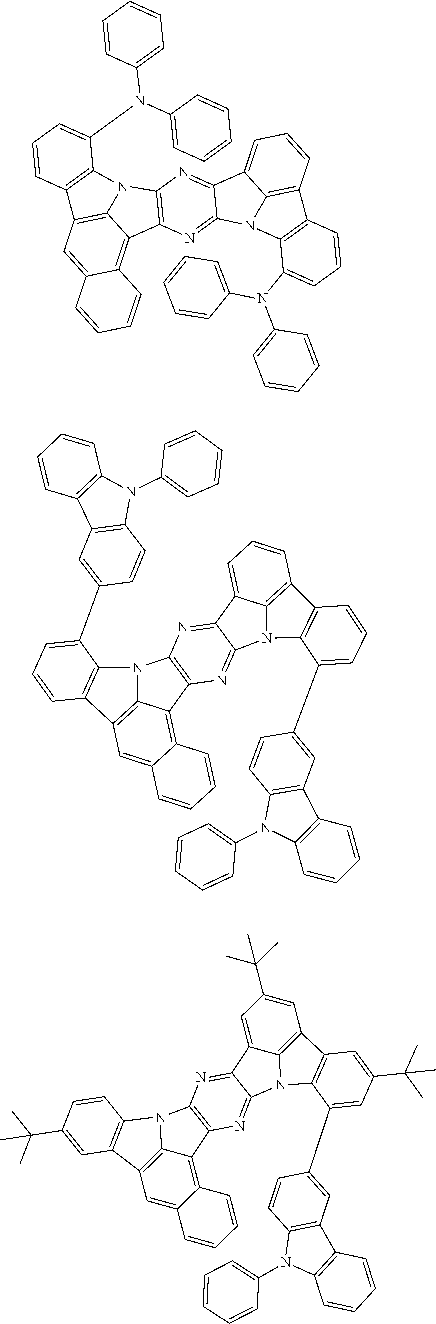 [SMILES: c1ccc(N(c2ccccc2)c2cccc3c4cccc5c6nc7c(nc6n(c23)c45)c2c3ccccc3cc3c4cccc(N(c5ccccc5)c5ccccc5)c4n7c32)cc1 ; c1ccc(-n2c3ccccc3c3cc(-c4cccc5c6cccc7c8nc9c(nc8n(c45)c67)c4c5ccccc5cc5c6cccc(-c7ccc8c(c7)c7ccccc7n8-c7ccccc7)c6n9c54)ccc32)cc1 ; CC(C)(C)c1ccc2c(c1)c1cc3ccccc3c3c4nc5c(nc4n2c13)c1cc(C(C)(C)C)cc2c3cc(C(C)(C)C)cc(-c4ccc6c(c4)c4ccccc4n6-c4ccccc4)c3n5c21]

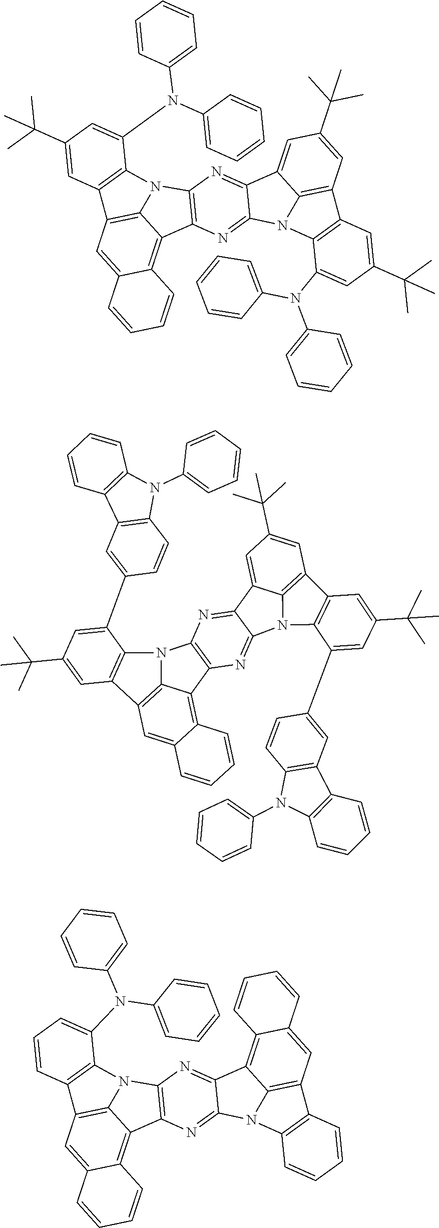

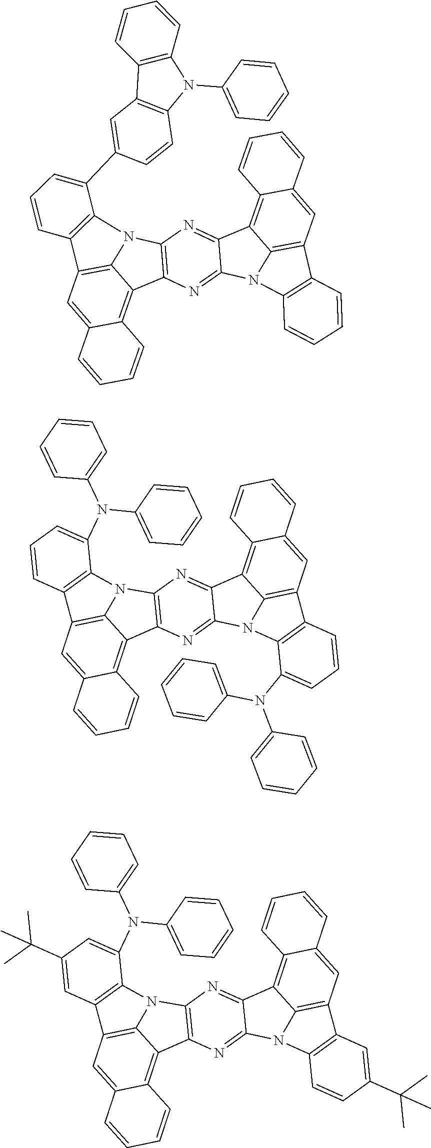

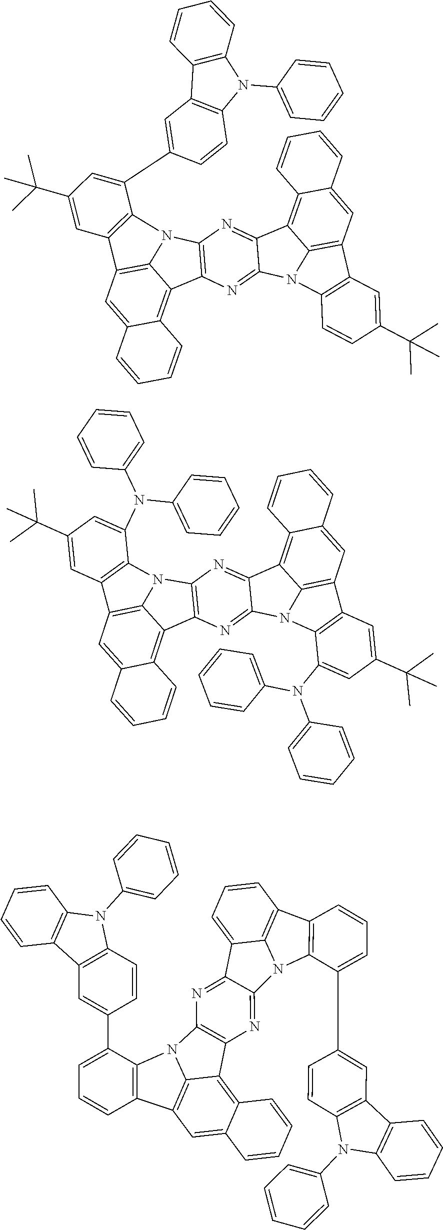

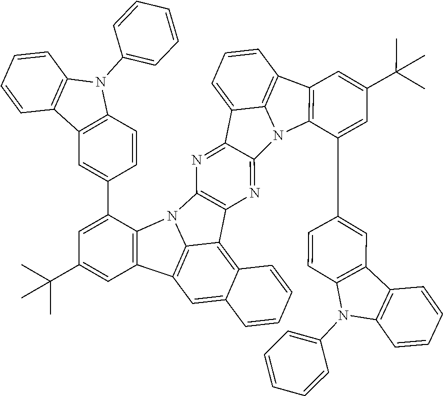 [SMILES: CC(C)(C)c1cc(-c2ccc3c(c2)c2ccccc2n3-c2ccccc2)c2c(c1)c1cccc3c4nc5c(nc4n2c13)c1c2ccccc2cc2c3cc(C(C)(C)C)cc(-c4ccc6c(c4)c4ccccc4n6-c4ccccc4)c3n5c21]

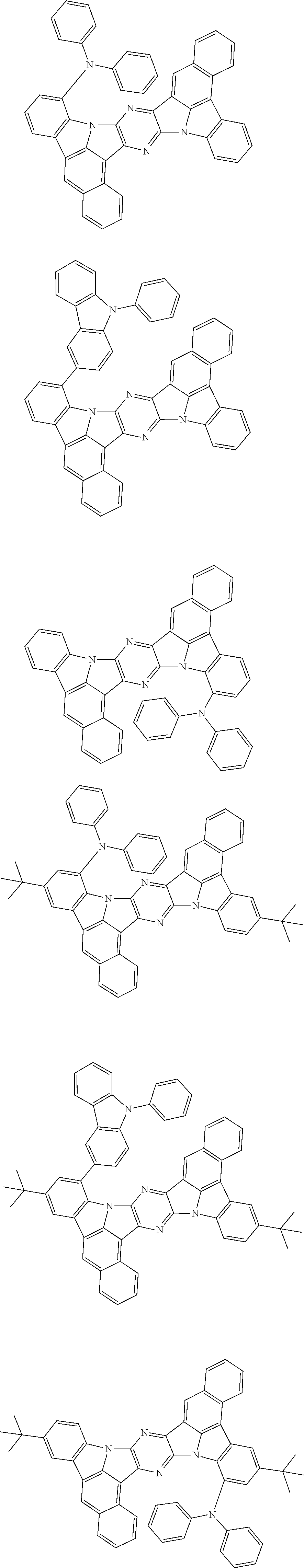

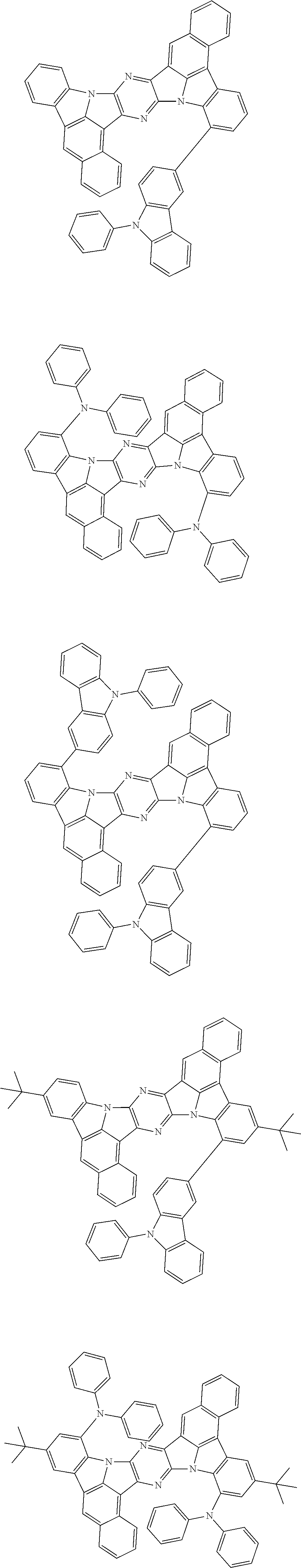

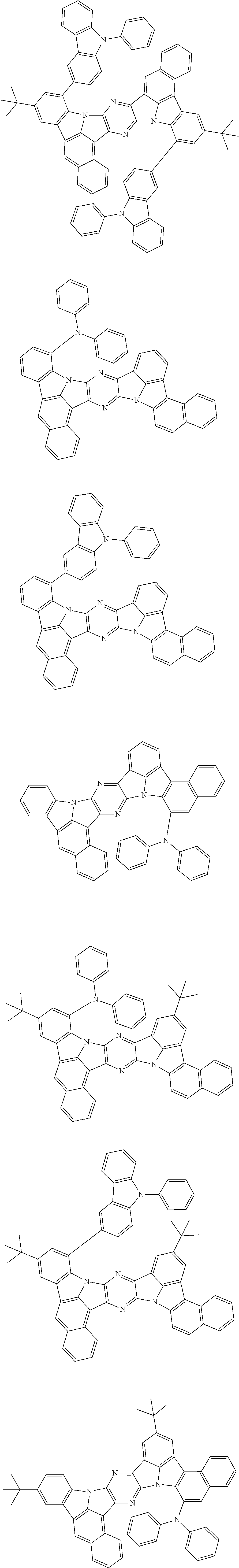

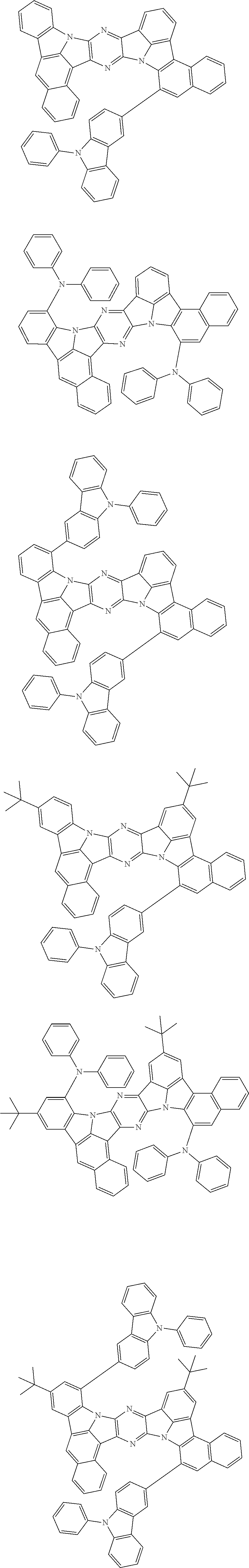

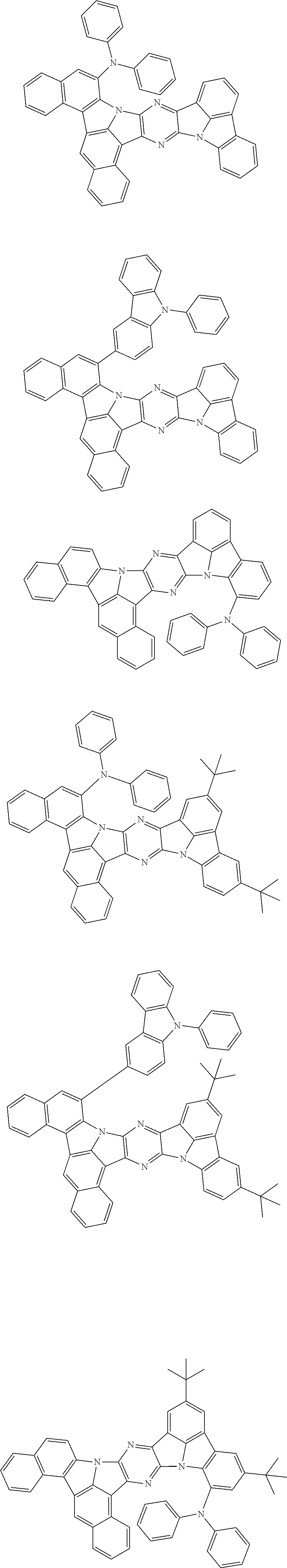

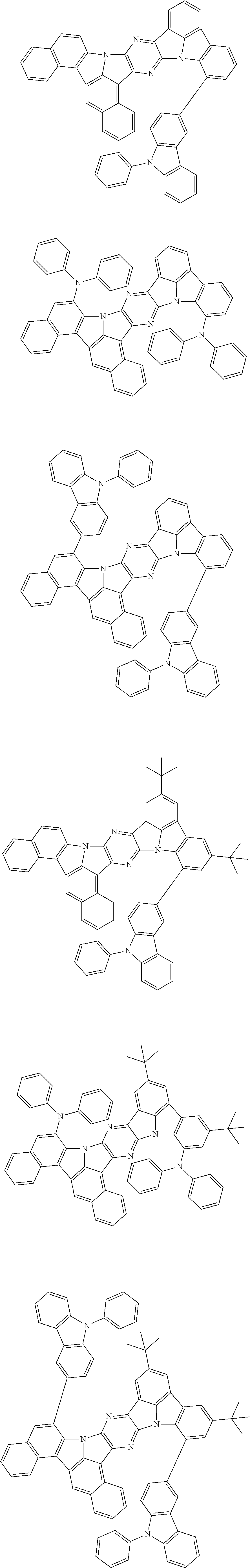

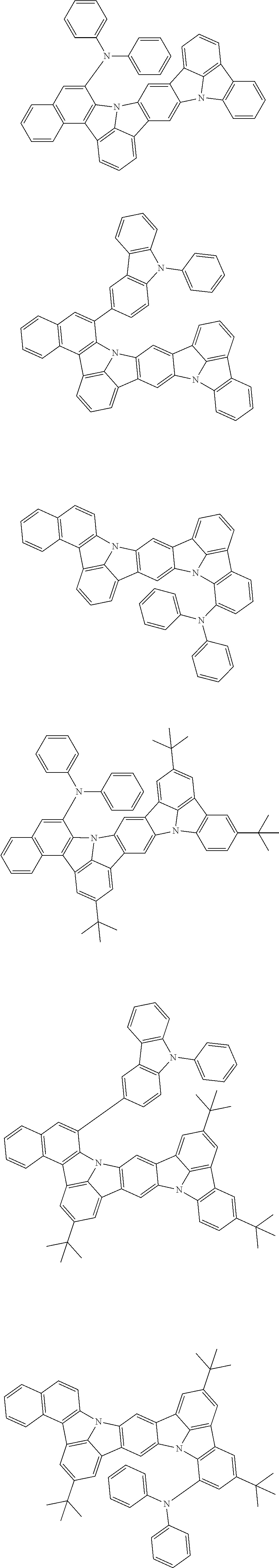

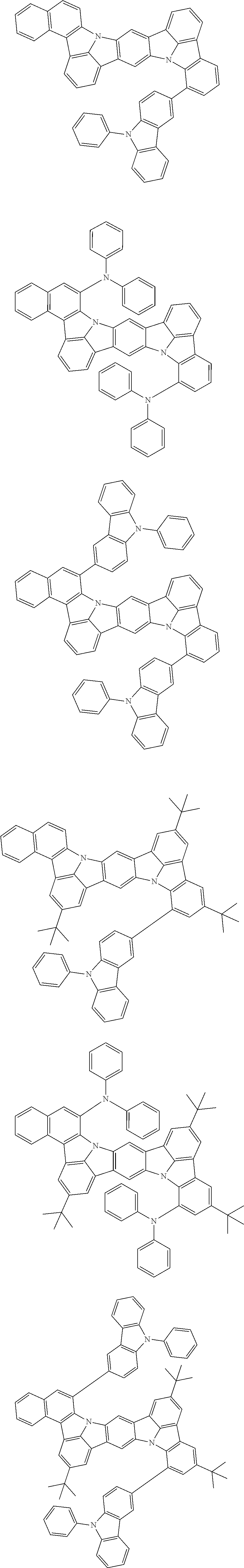

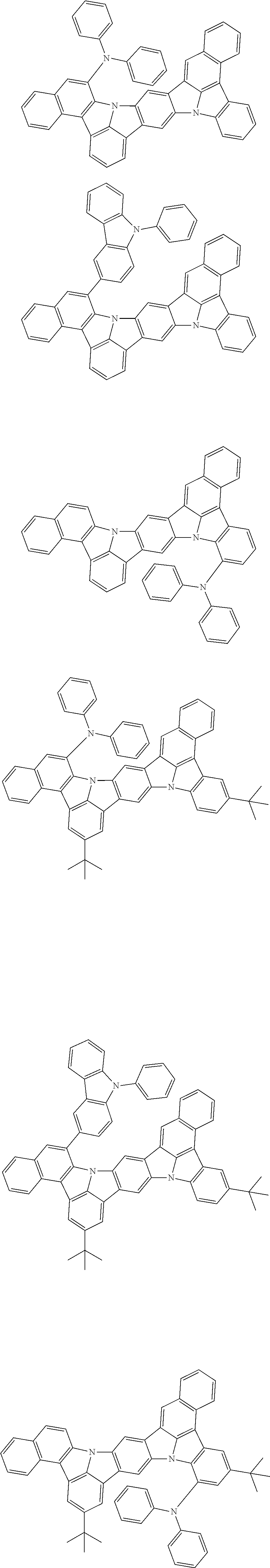

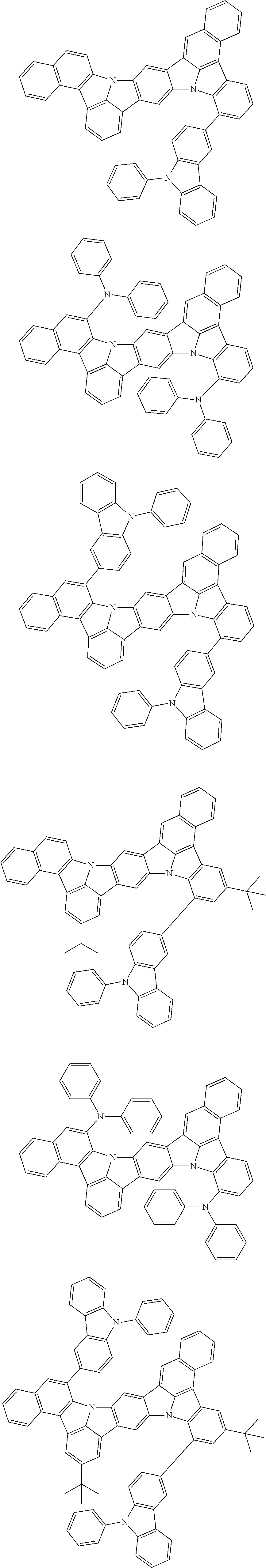

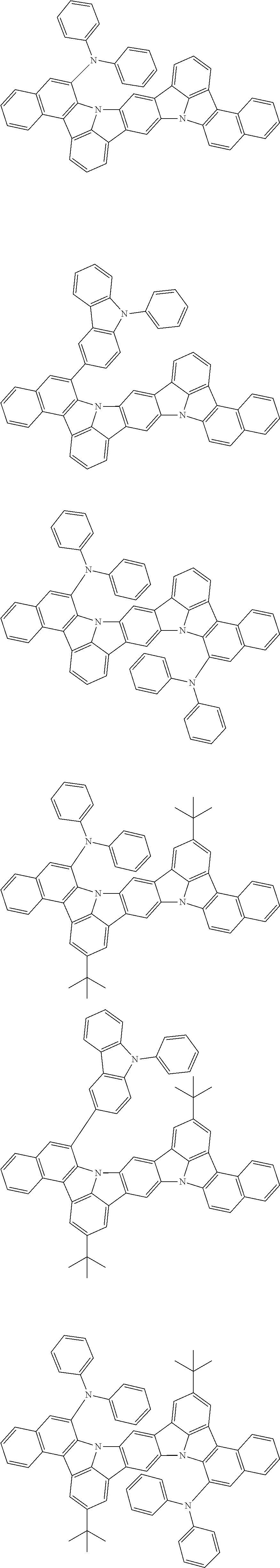

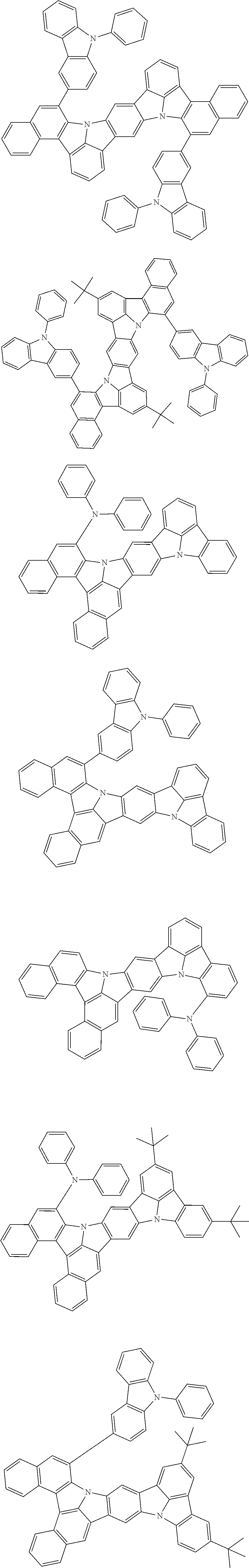

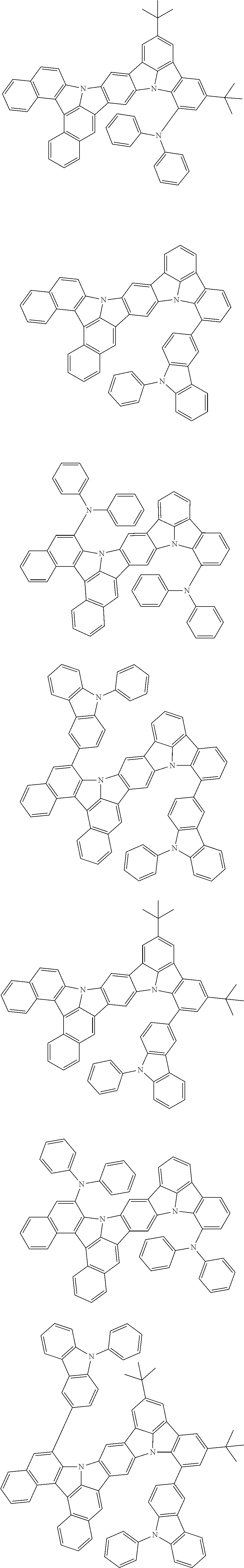

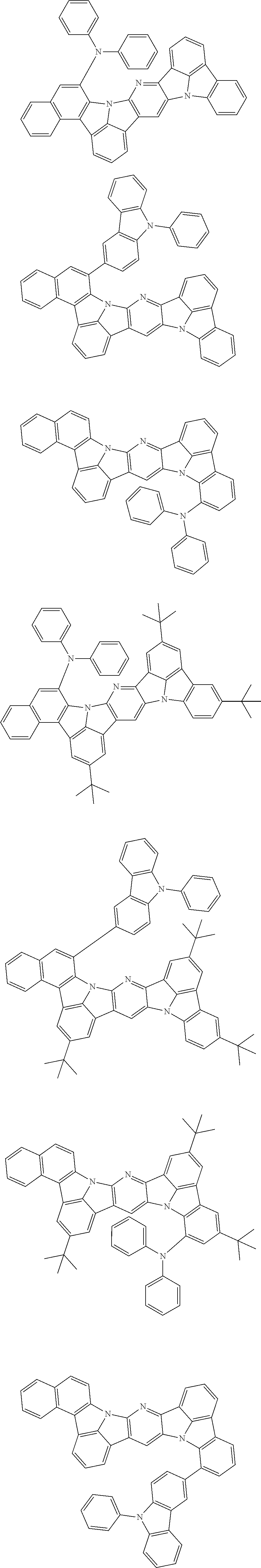

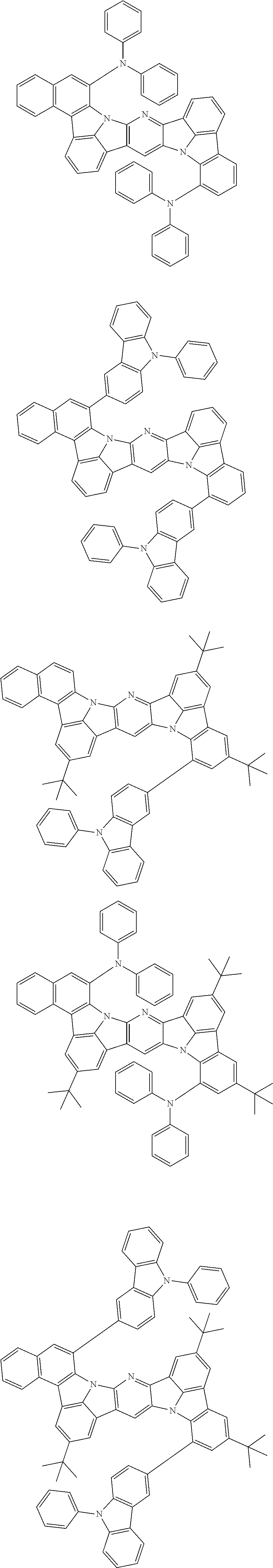

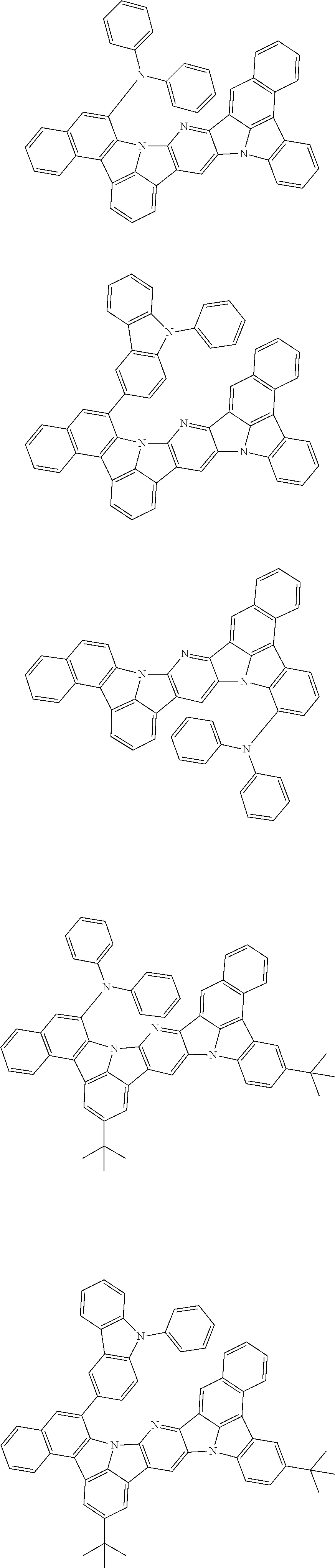

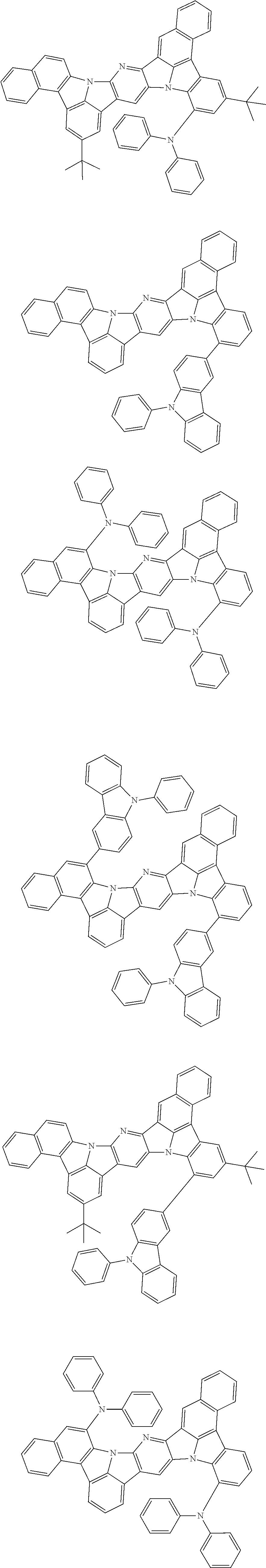

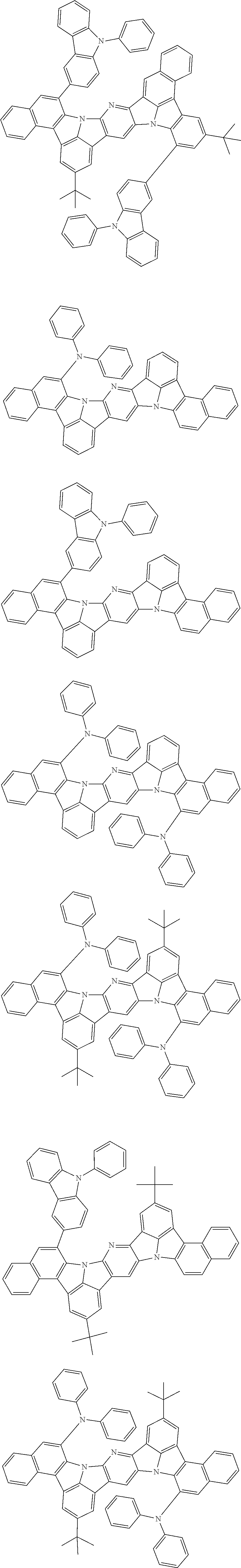

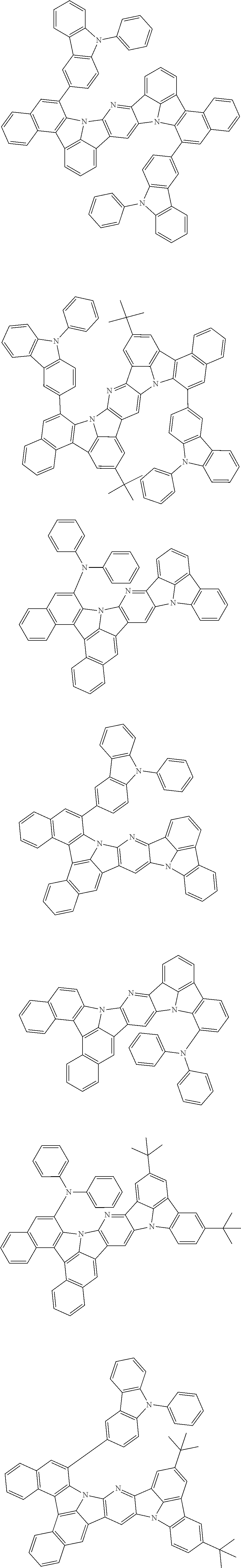

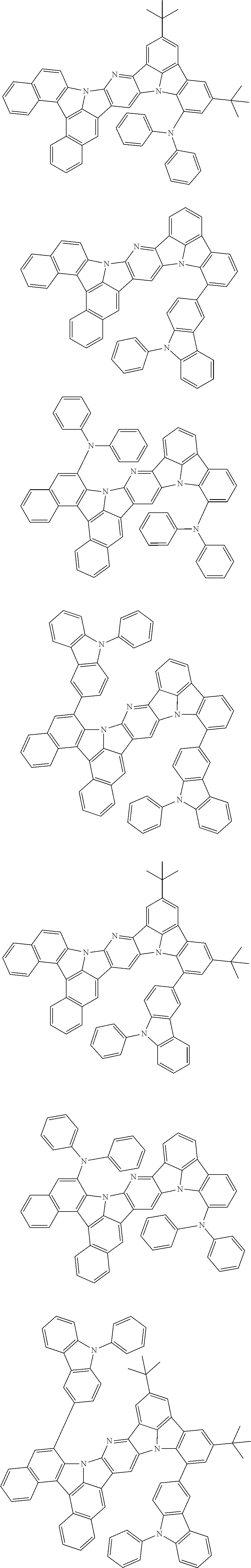

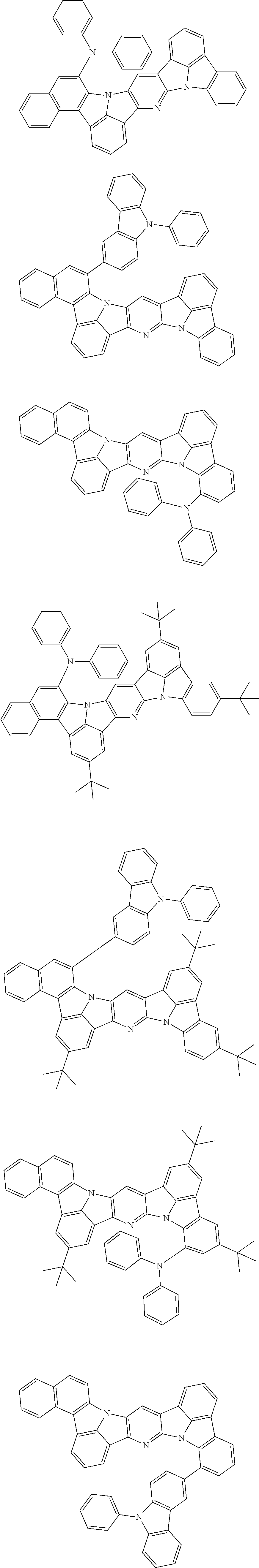

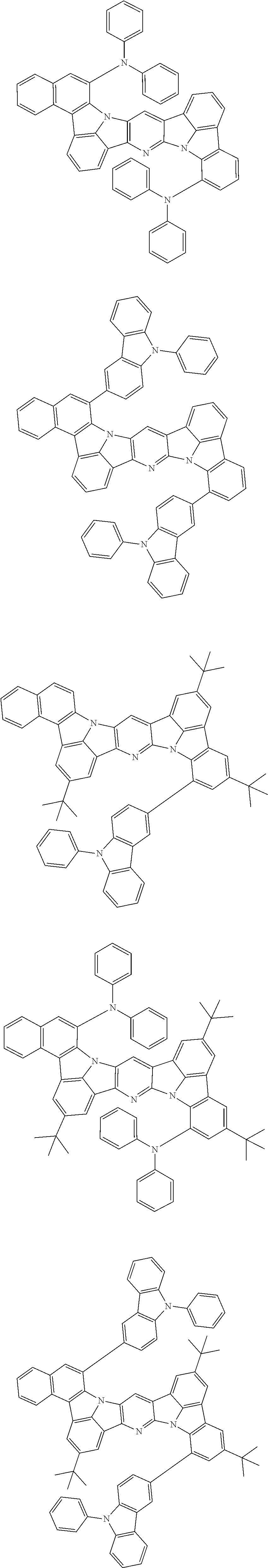

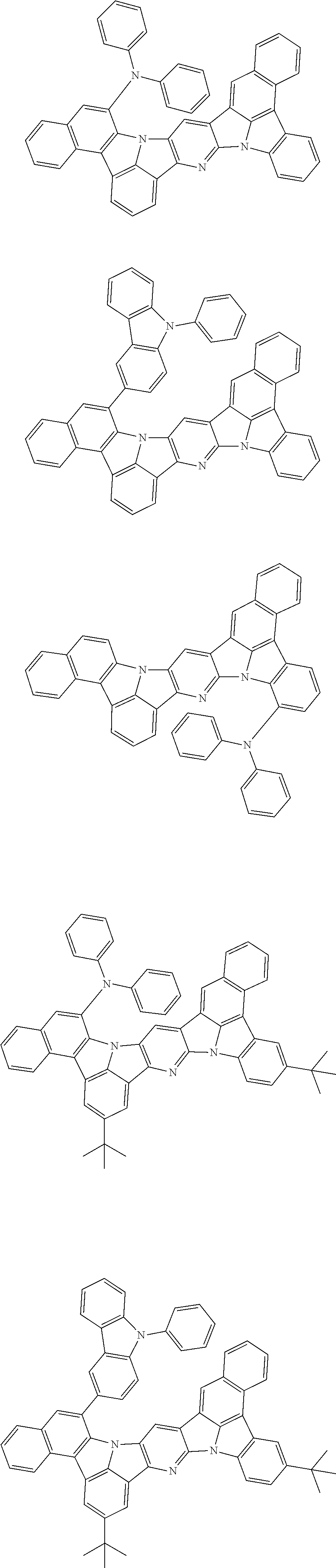

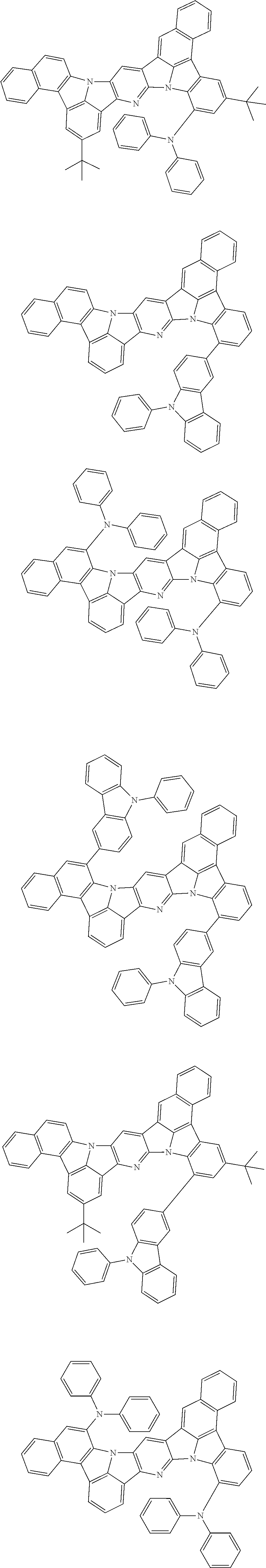

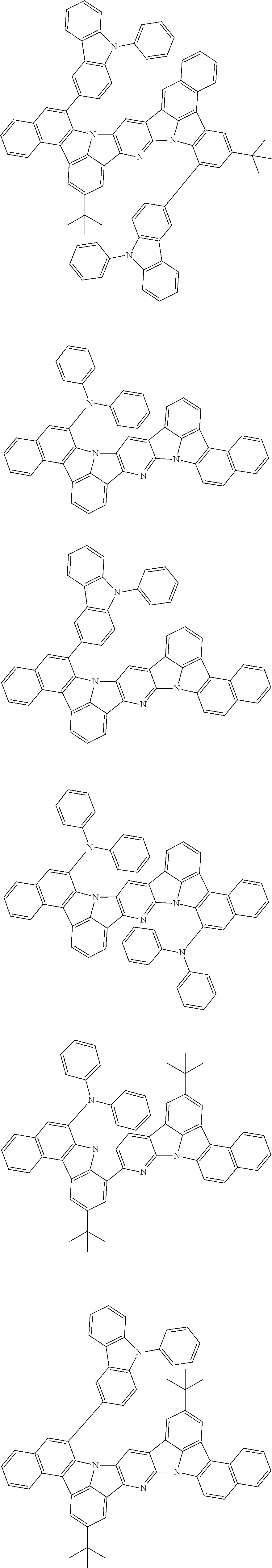

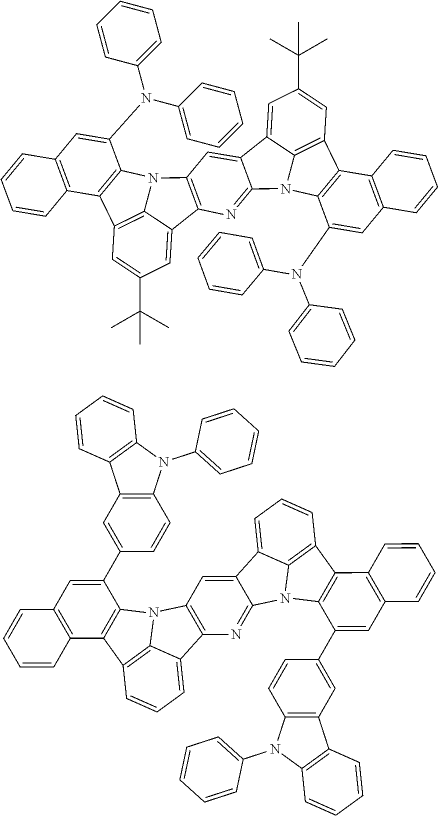 [SMILES: CC(C)(C)c1cc2c3nc4c(cc3n3c5c(N(c6ccccc6)c6ccccc6)cc6ccccc6c5c(c1)c23)c1cc(C(C)(C)C)cc2c3c5ccccc5cc(N(c5ccccc5)c5ccccc5)c3n4c12 ; c1ccc(-n2c3ccccc3c3cc(-c4cc5ccccc5c5c6cccc7c8nc9c(cc8n(c45)c76)c4cccc5c6c7ccccc7cc(-c7ccc8c(c7)c7ccccc7n8-c7ccccc7)c6n9c45)ccc32)cc1]

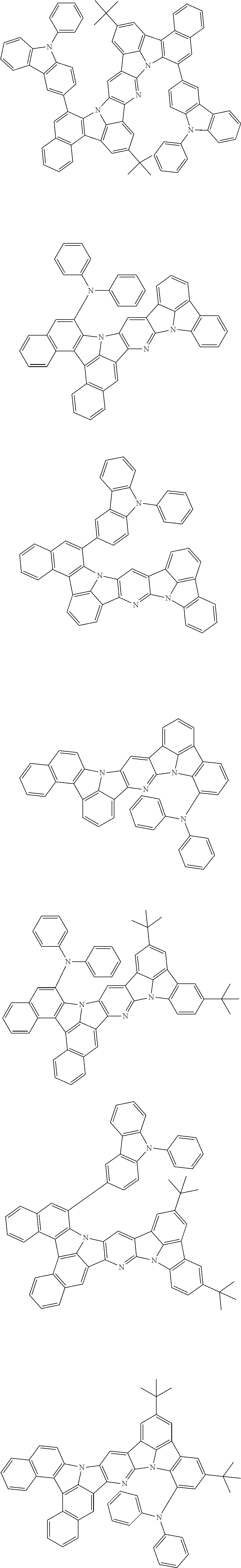

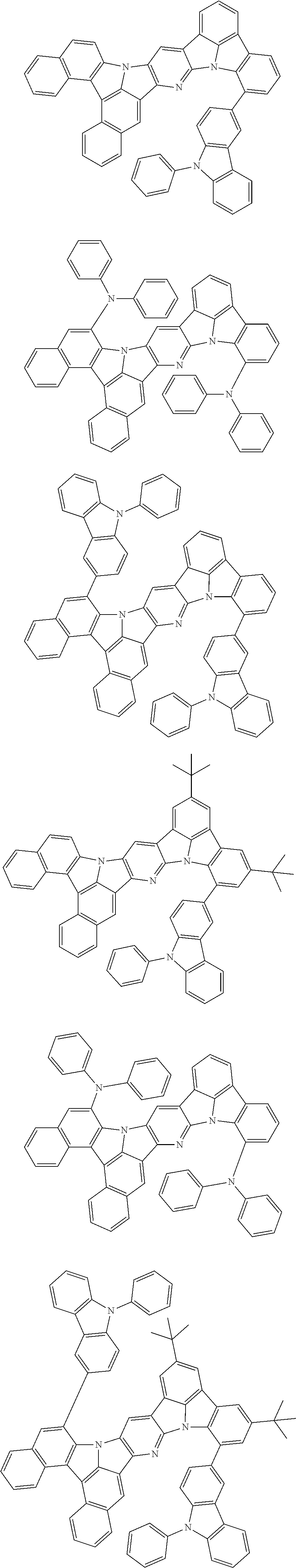

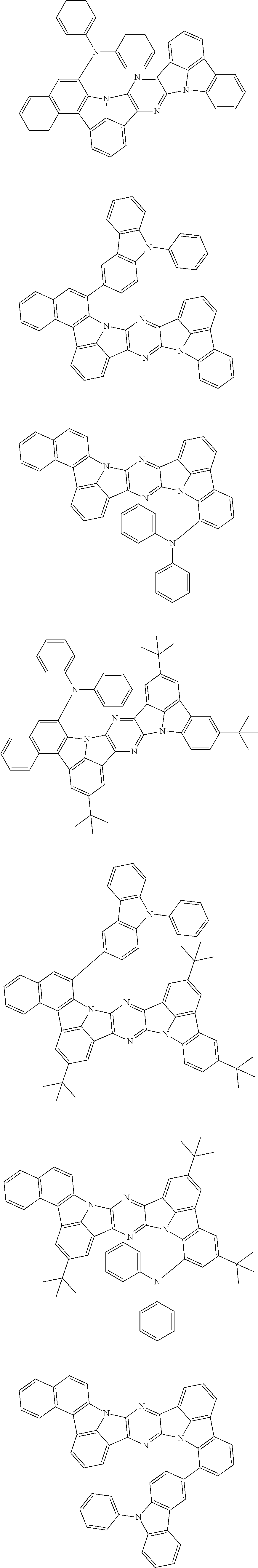

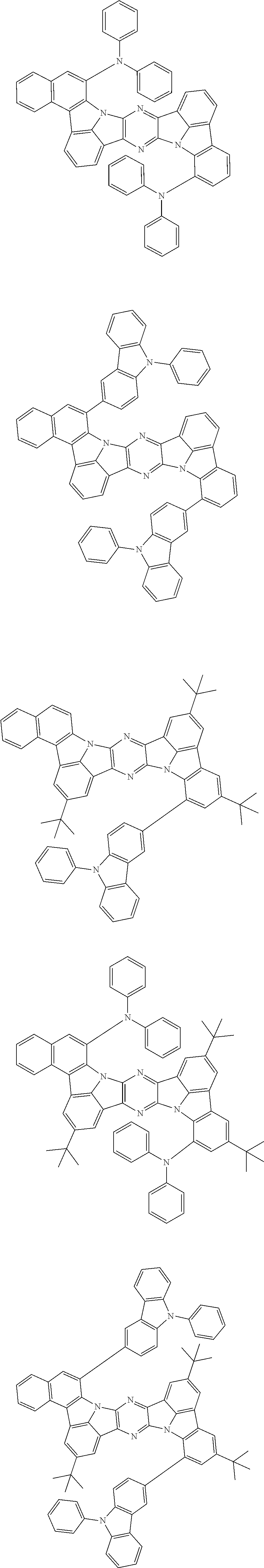

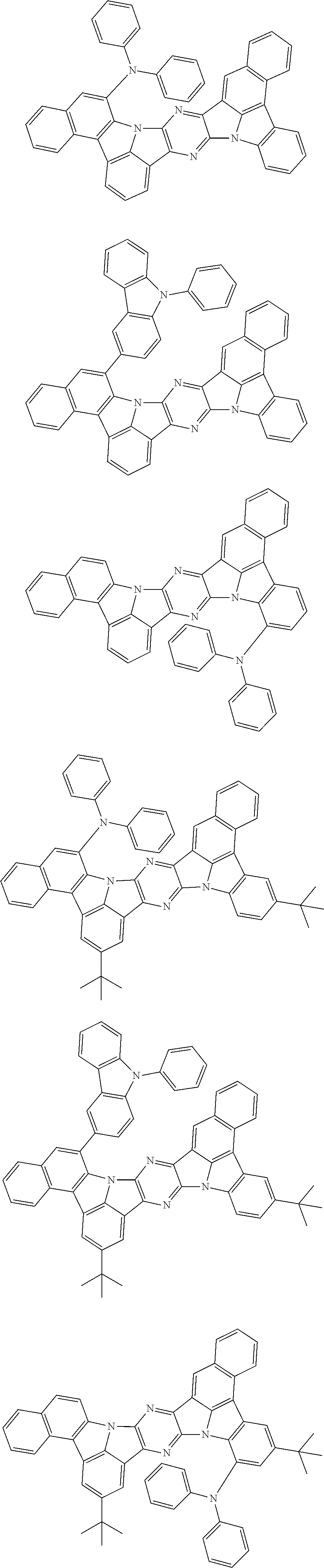

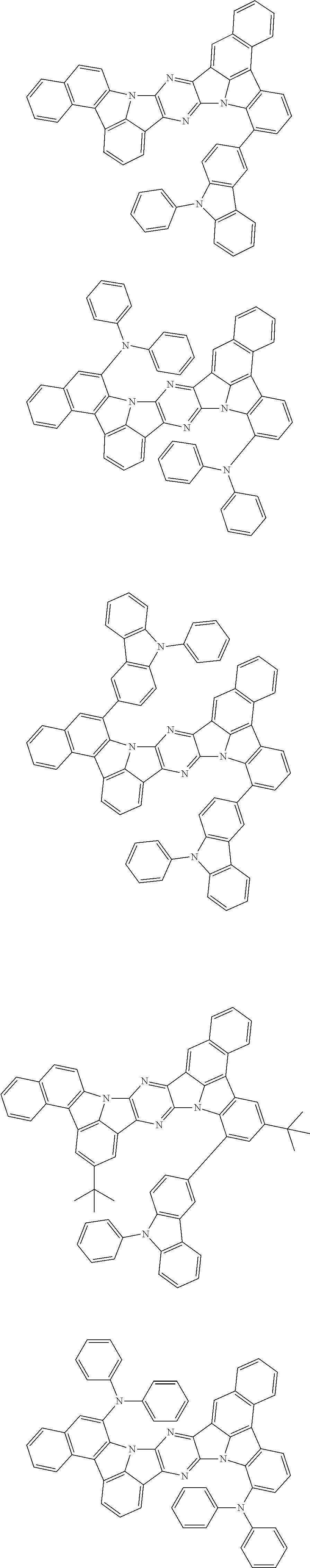

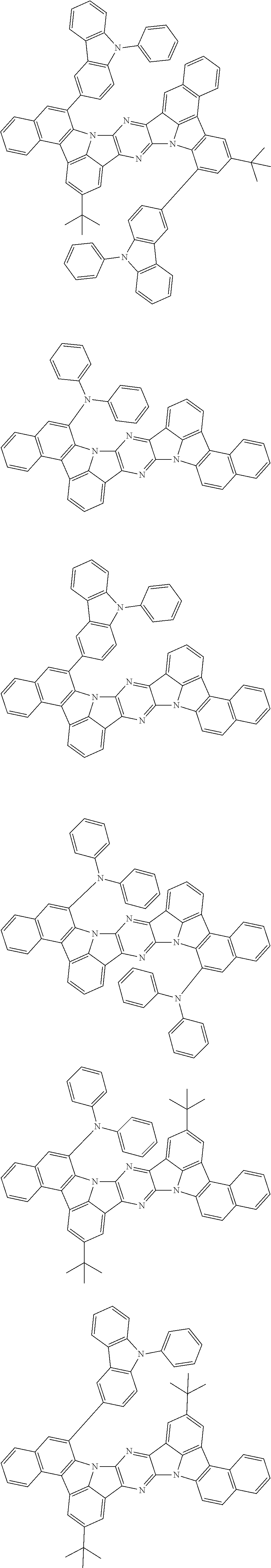

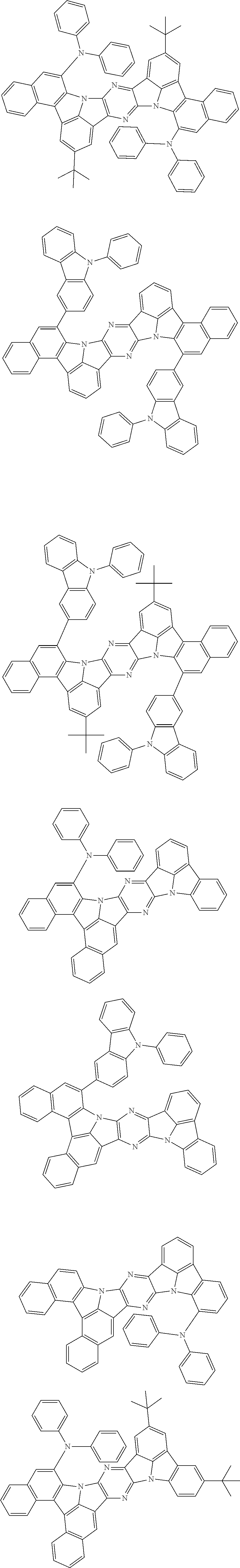

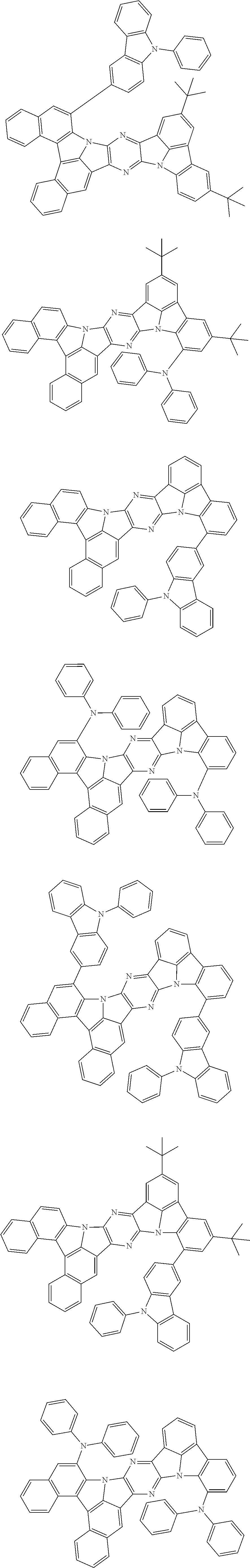

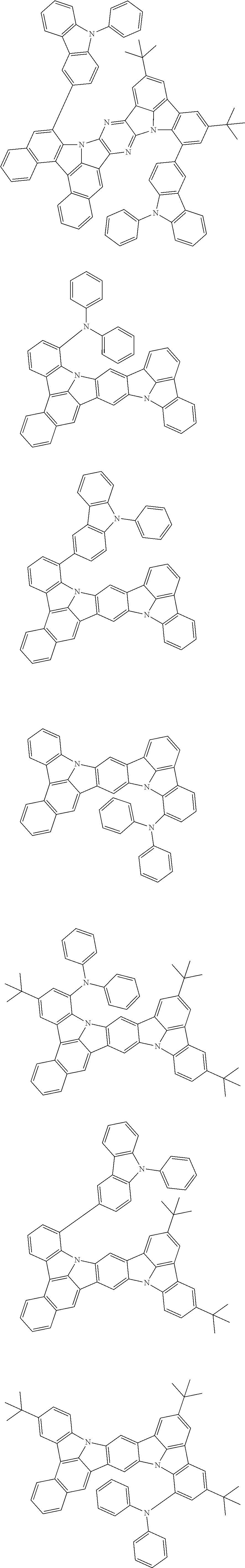

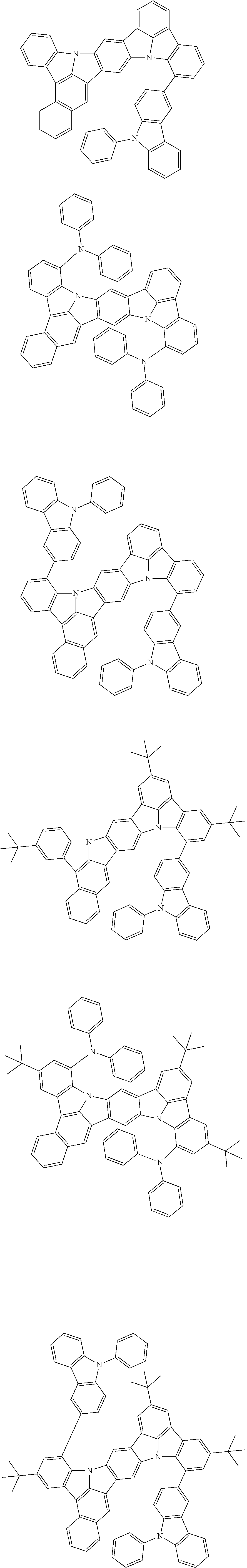

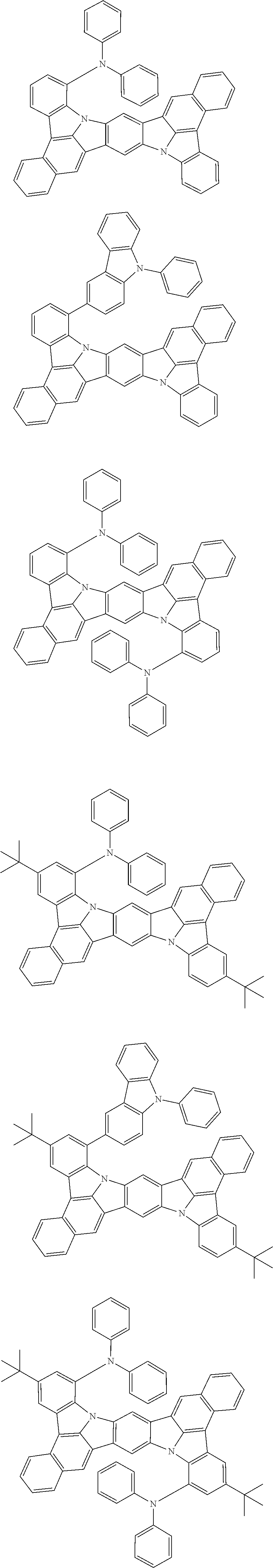

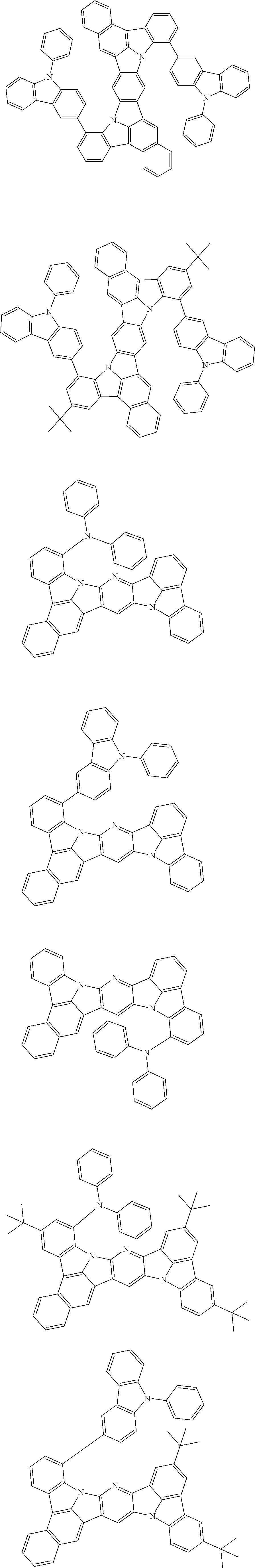

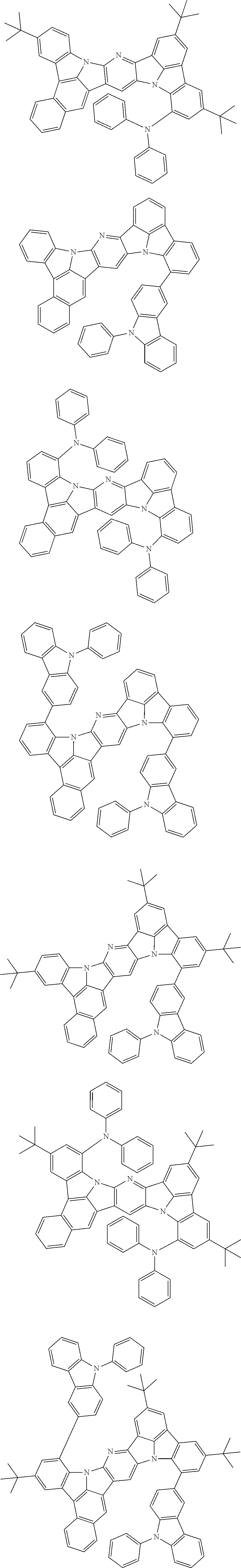

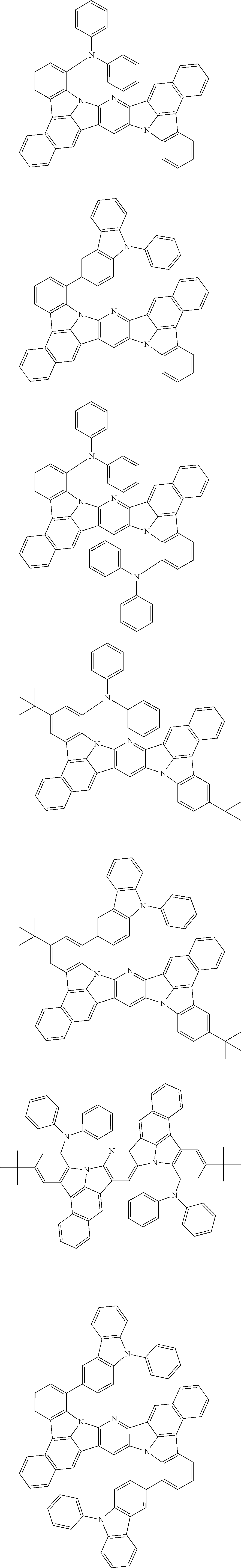

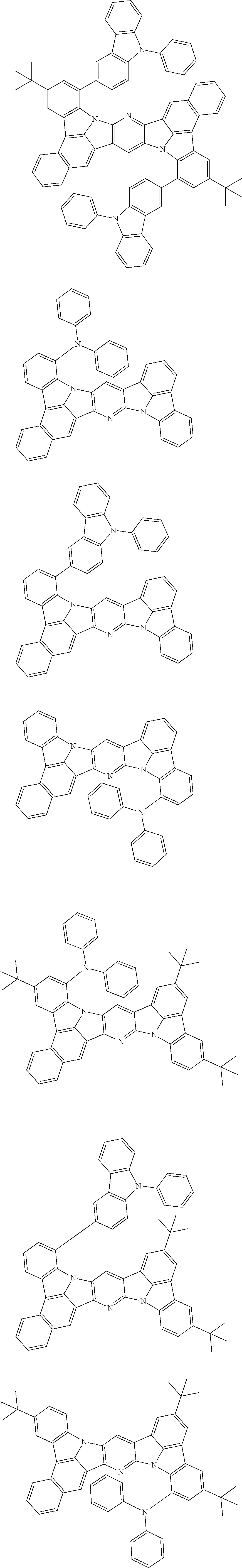

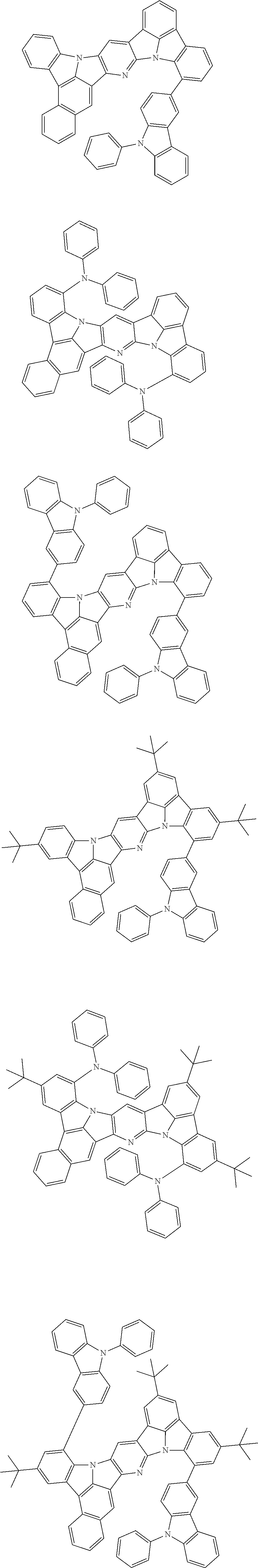

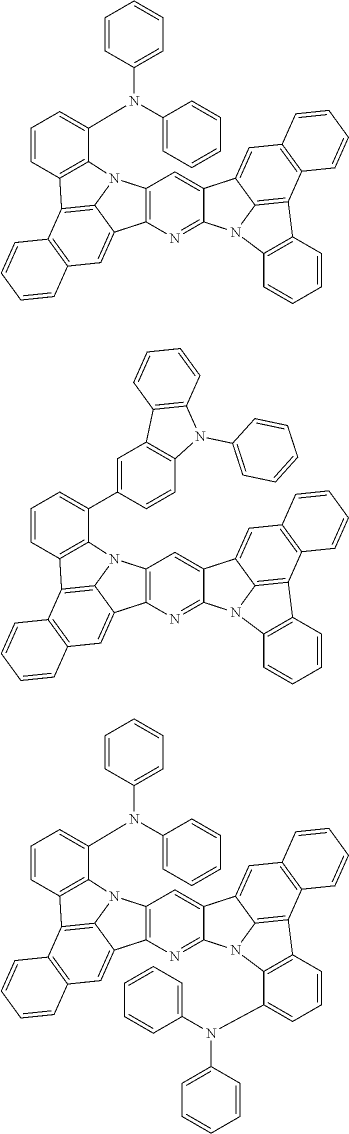 [SMILES: c1ccc(N(c2ccccc2)c2cccc3c4c5ccccc5cc5c6nc7c(cc6n(c23)c54)c2cc3ccccc3c3c4ccccc4n7c23)cc1 ; c1ccc(-n2c3ccccc3c3cc(-c4cccc5c6c7ccccc7cc7c8nc9c(cc8n(c45)c76)c4cc5ccccc5c5c6ccccc6n9c45)ccc32)cc1 ; c1ccc(N(c2ccccc2)c2cccc3c4c5ccccc5cc5c6nc7c(cc6n(c23)c54)c2cc3ccccc3c3c4cccc(N(c5ccccc5)c5ccccc5)c4n7c23)cc1]

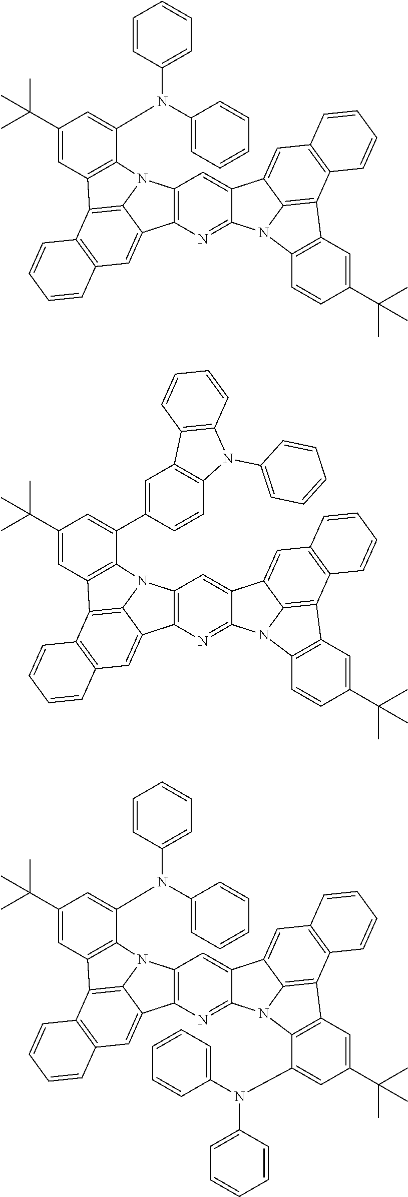

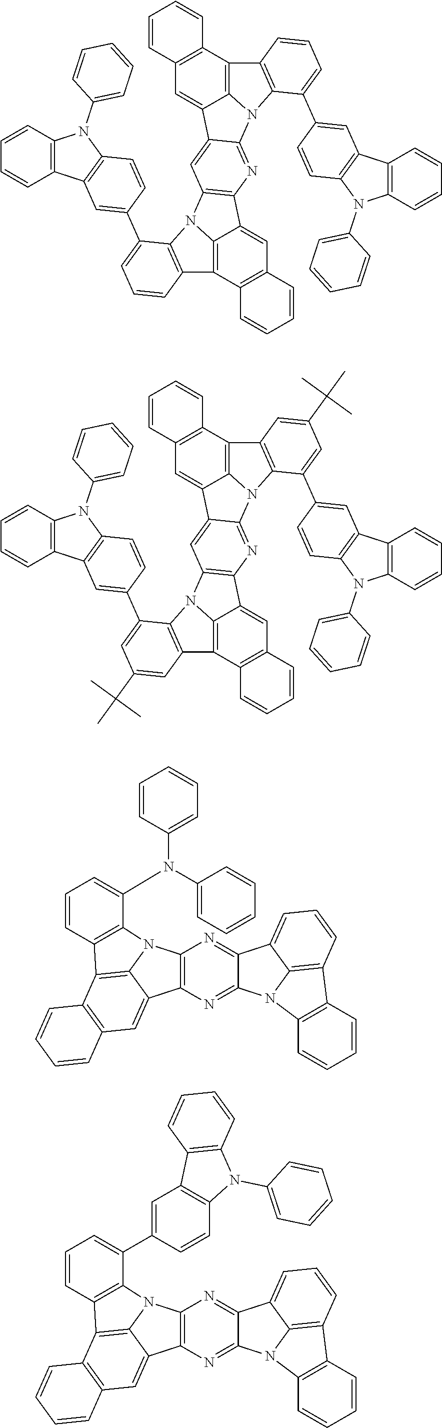 [SMILES: c1ccc(-n2c3ccccc3c3cc(-c4cccc5c6c7ccccc7cc7c8nc9c(cc8n(c45)c76)c4cc5ccccc5c5c6cccc(-c7ccc8c(c7)c7ccccc7n8-c7ccccc7)c6n9c45)ccc32)cc1 ; CC(C)(C)c1cc(-c2ccc3c(c2)c2ccccc2n3-c2ccccc2)c2c(c1)c1c3ccccc3cc3c4nc5c(cc4n2c31)c1cc2ccccc2c2c3cc(C(C)(C)C)cc(-c4ccc6c(c4)c4ccccc4n6-c4ccccc4)c3n5c12 ; c1ccc(N(c2ccccc2)c2cccc3c4c5ccccc5cc5c6nc7c(nc6n(c23)c54)c2cccc3c4ccccc4n7c32)cc1 ; c1ccc(-n2c3ccccc3c3cc(-c4cccc5c6c7ccccc7cc7c8nc9c(nc8n(c45)c76)c4cccc5c6ccccc6n9c54)ccc32)cc1]

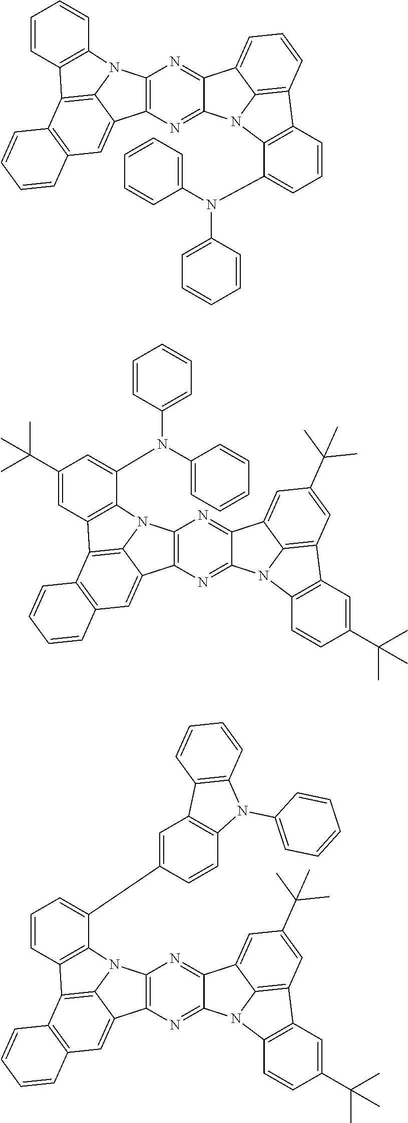 [SMILES: c1ccc(N(c2ccccc2)c2cccc3c4cccc5c6nc7c(nc6n(c23)c45)c2cc3ccccc3c3c4ccccc4n7c23)cc1 ; CC(C)(C)c1ccc2c(c1)c1cc(C(C)(C)C)cc3c4nc5c(nc4n2c13)c1cc2ccccc2c2c3cc(C(C)(C)C)cc(N(c4ccccc4)c4ccccc4)c3n5c12 ; CC(C)(C)c1ccc2c(c1)c1cc(C(C)(C)C)cc3c4nc5c(nc4n2c13)c1cc2ccccc2c2c3cccc(-c4ccc6c(c4)c4ccccc4n6-c4ccccc4)c3n5c12]

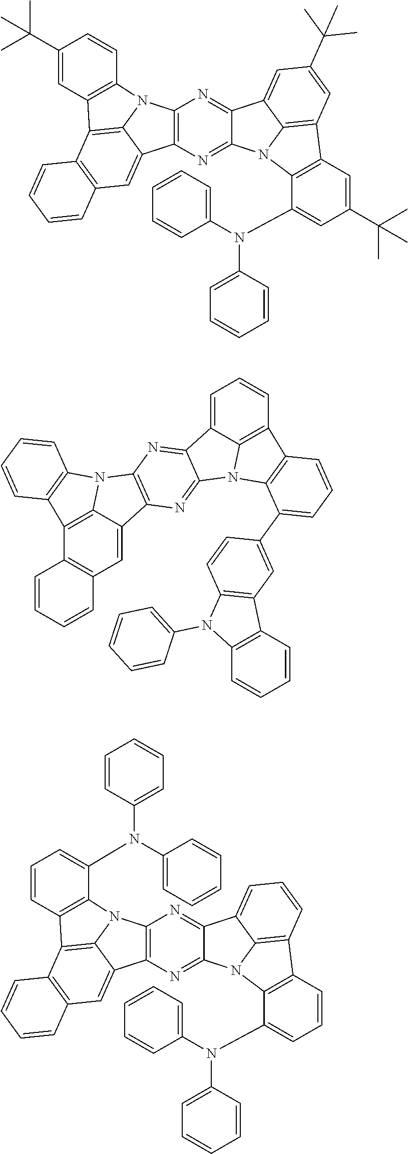

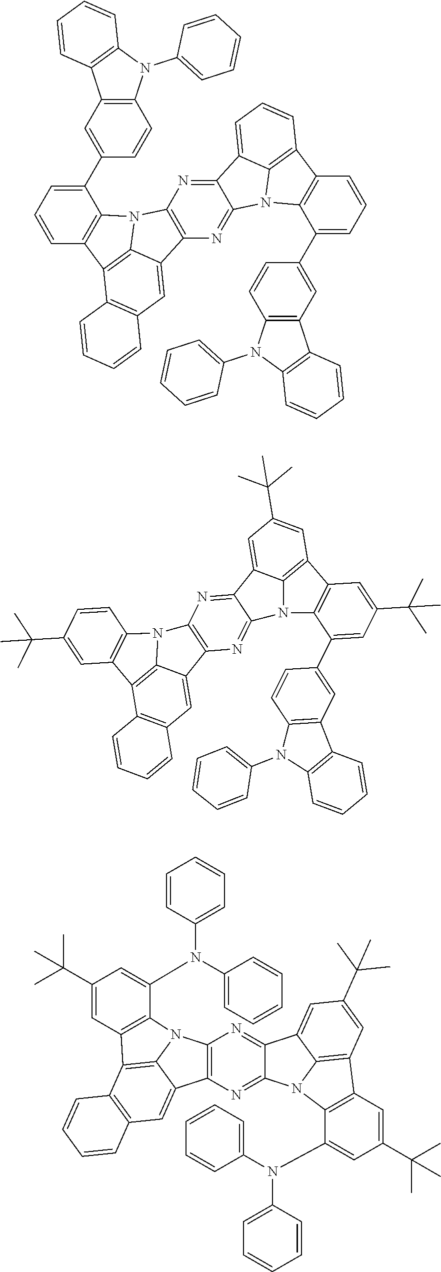

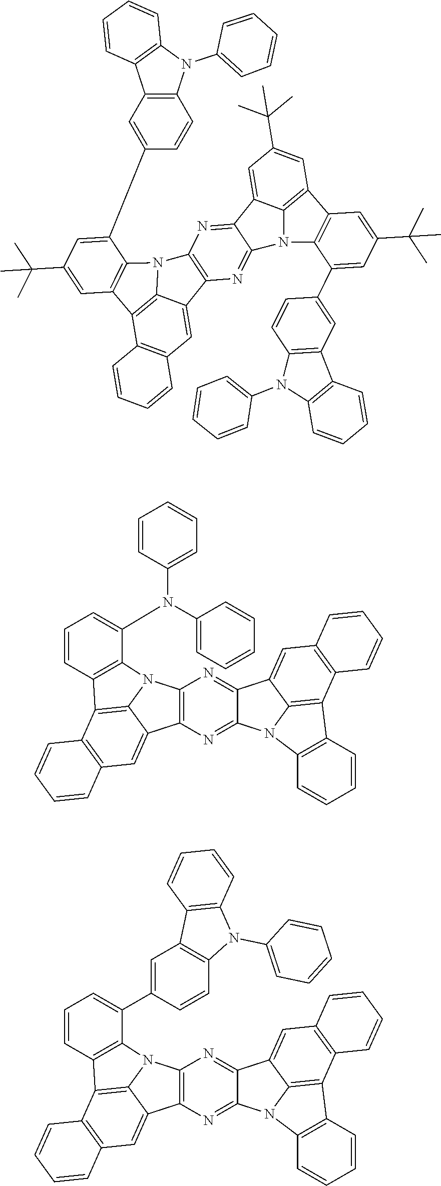 [SMILES: CC(C)(C)c1cc(-c2ccc3c(c2)c2ccccc2n3-c2ccccc2)c2c(c1)c1cc(C(C)(C)C)cc3c4nc5c(nc4n2c13)c1cc2ccccc2c2c3cc(C(C)(C)C)cc(-c4ccc6c(c4)c4ccccc4n6-c4ccccc4)c3n5c12 ; c1ccc(N(c2ccccc2)c2cccc3c4c5ccccc5cc5c6nc7c(nc6n(c23)c54)c2cc3ccccc3c3c4ccccc4n7c23)cc1 ; c1ccc(-n2c3ccccc3c3cc(-c4cccc5c6c7ccccc7cc7c8nc9c(nc8n(c45)c76)c4cc5ccccc5c5c6ccccc6n9c45)ccc32)cc1]

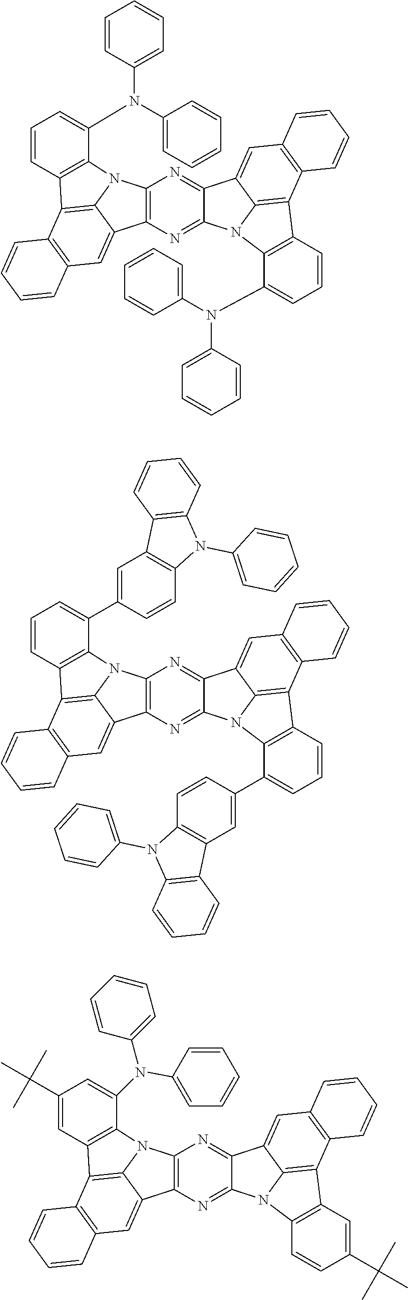 [SMILES: c1ccc(N(c2ccccc2)c2cccc3c4c5ccccc5cc5c6nc7c(nc6n(c23)c54)c2cc3ccccc3c3c4cccc(N(c5ccccc5)c5ccccc5)c4n7c23)cc1 ; c1ccc(-n2c3ccccc3c3cc(-c4cccc5c6c7ccccc7cc7c8nc9c(nc8n(c45)c76)c4cc5ccccc5c5c6cccc(-c7ccc8c(c7)c7ccccc7n8-c7ccccc7)c6n9c45)ccc32)cc1 ; CC(C)(C)c1ccc2c(c1)c1c3ccccc3cc3c4nc5c(nc4n2c31)c1cc2ccccc2c2c3cc(C(C)(C)C)cc(N(c4ccccc4)c4ccccc4)c3n5c12]

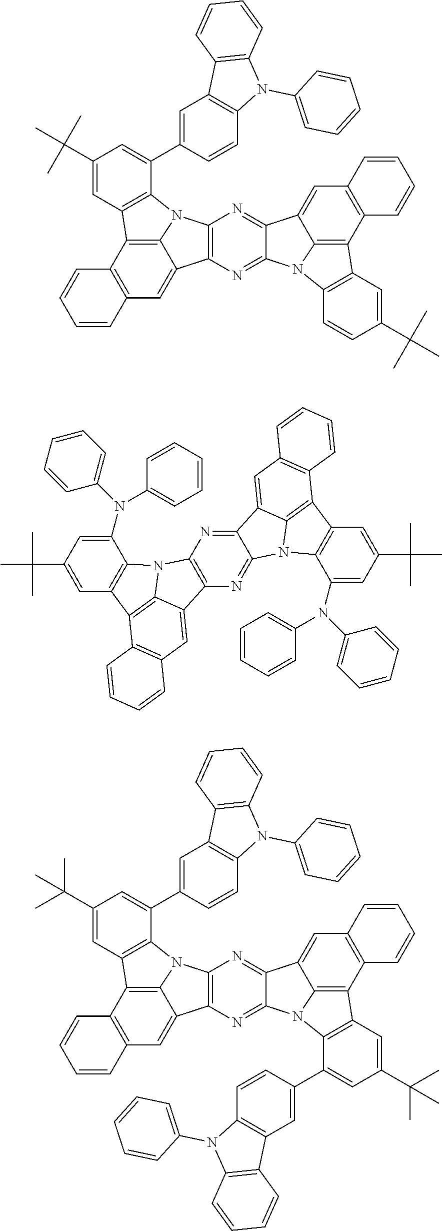

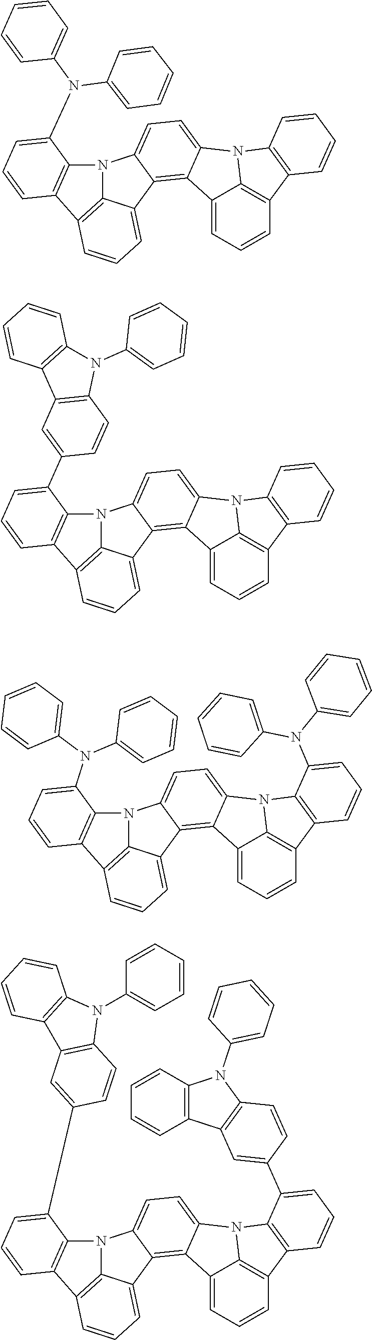

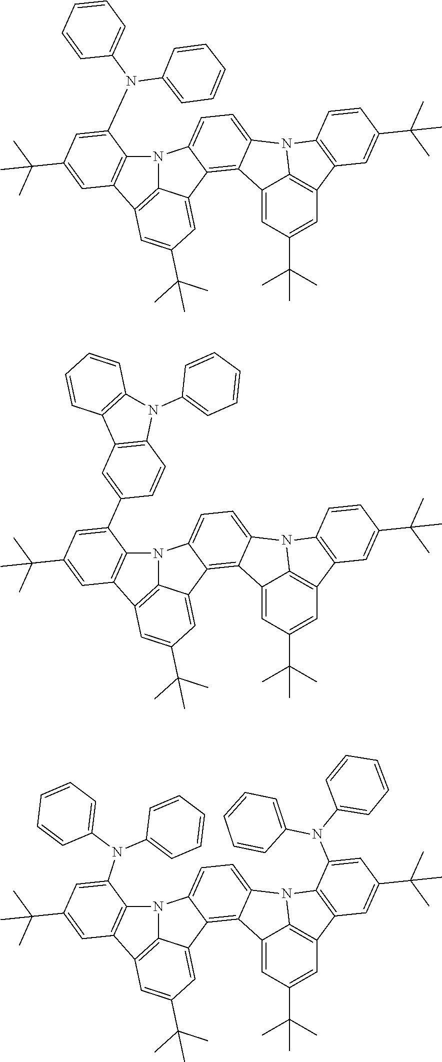

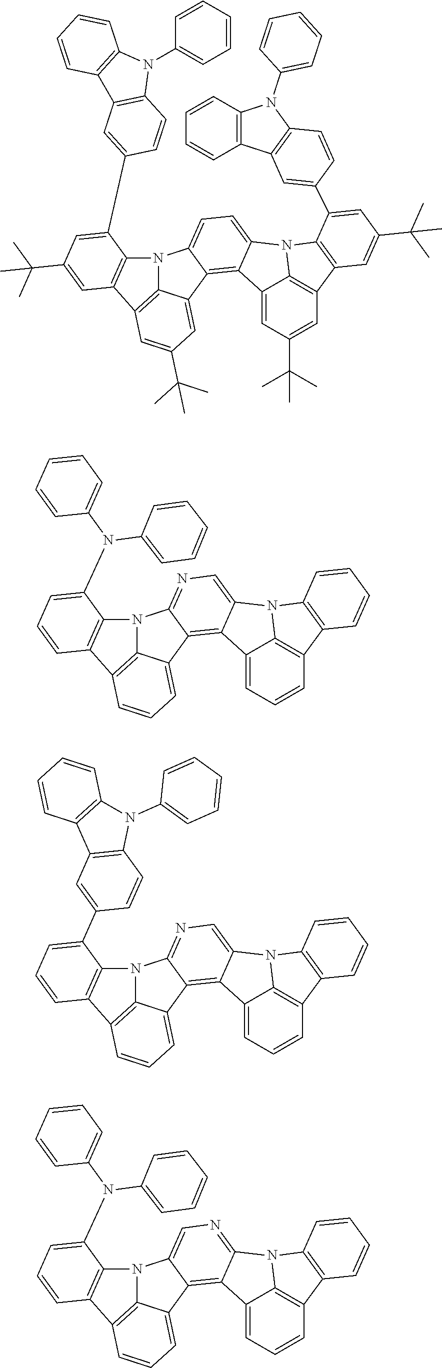 [SMILES: CC(C)(C)c1cc(-c2ccc3c(c2)c2ccccc2n3-c2ccccc2)c2c(c1)c1cc(C(C)(C)C)cc3c4c5c6cc(C(C)(C)C)cc7c8cc(C(C)(C)C)cc(-c9ccc%10c(c9)c9ccccc9n%10-c9ccccc9)c8n(c5ccc4n2c13)c76 ; c1ccc(N(c2ccccc2)c2cccc3c4cccc5c6c7c8cccc9c%10ccccc%10n(c7cnc6n(c23)c45)c98)cc1 ; c1ccc(-n2c3ccccc3c3cc(-c4cccc5c6cccc7c8c9c%10cccc%11c%12ccccc%12n(c9cnc8n(c45)c67)c%11%10)ccc32)cc1 ; c1ccc(N(c2ccccc2)c2cccc3c4cccc5c6c7c8cccc9c%10ccccc%10n(c7ncc6n(c23)c45)c98)cc1]

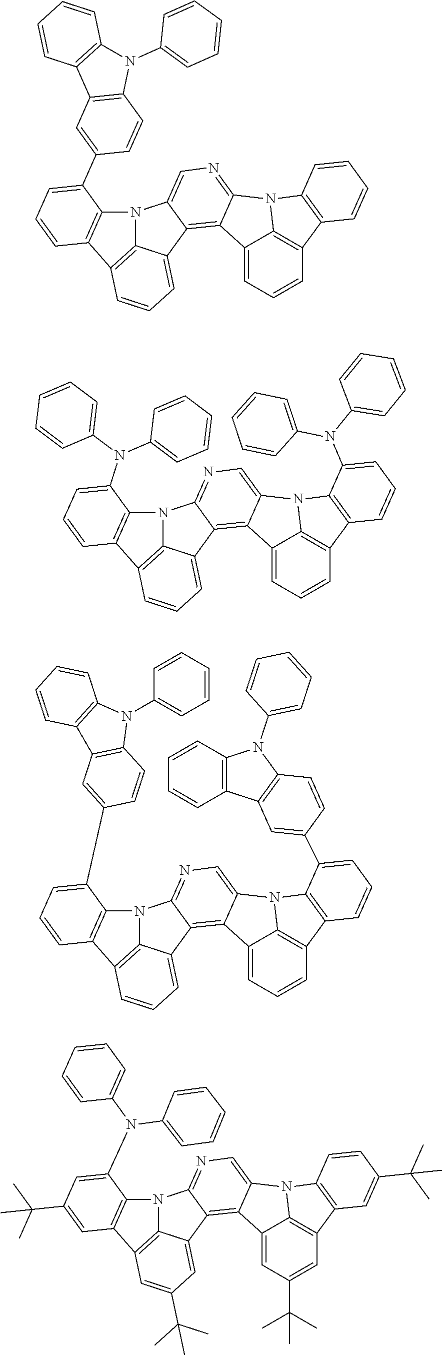

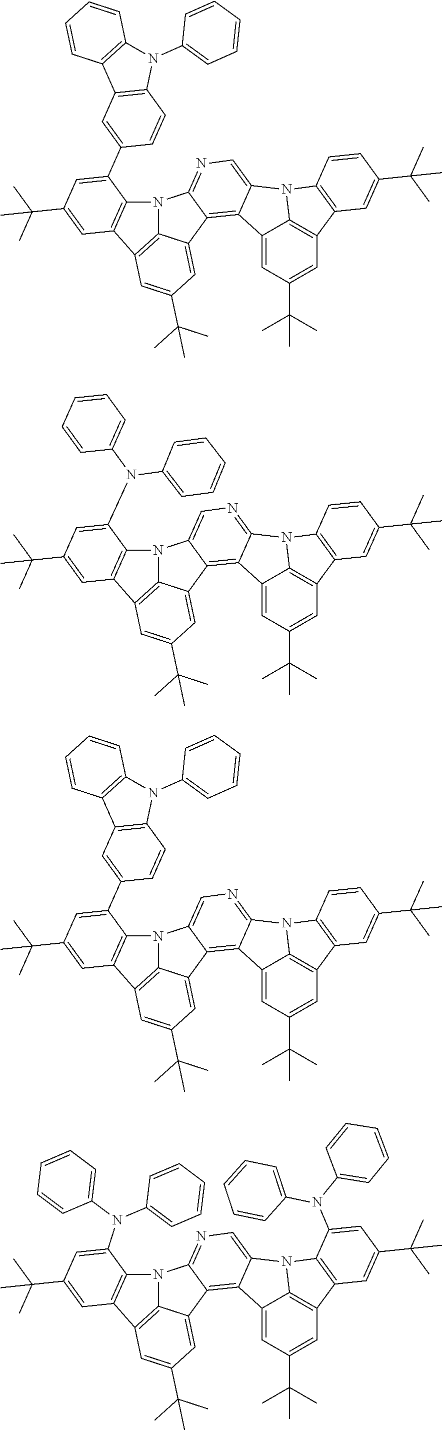

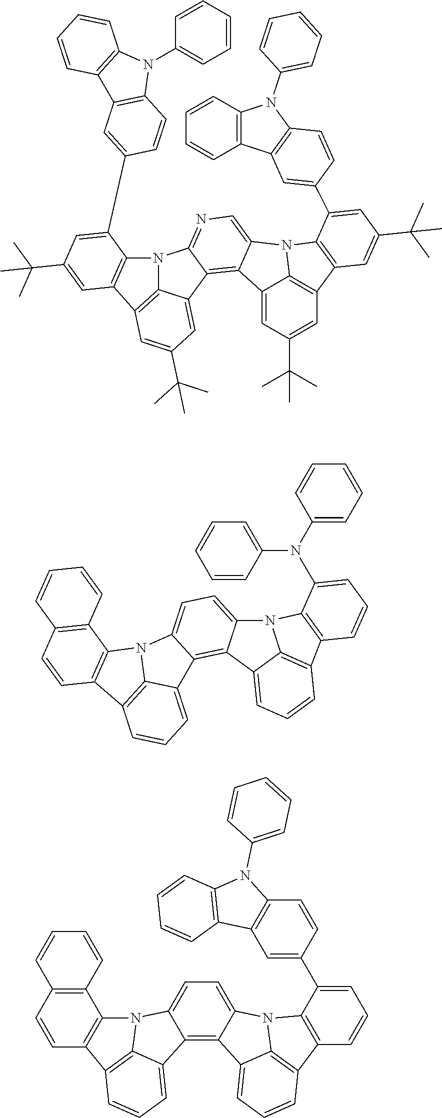 [SMILES: CC(C)(C)c1cc(-c2ccc3c(c2)c2ccccc2n3-c2ccccc2)c2c(c1)c1cc(C(C)(C)C)cc3c4c5c6cc(C(C)(C)C)cc7c8cc(C(C)(C)C)cc(-c9ccc%10c(c9)c9ccccc9n%10-c9ccccc9)c8n(c5ncc4n2c13)c76 ; c1ccc(N(c2ccccc2)c2cccc3c4cccc5c6c7c8cccc9c%10ccc%11ccccc%11c%10n(c7ccc6n(c23)c45)c98)cc1 ; c1ccc(-n2c3ccccc3c3cc(-c4cccc5c6cccc7c8c9c%10cccc%11c%12ccc%13ccccc%13c%12n(c9ccc8n(c45)c67)c%11%10)ccc32)cc1]

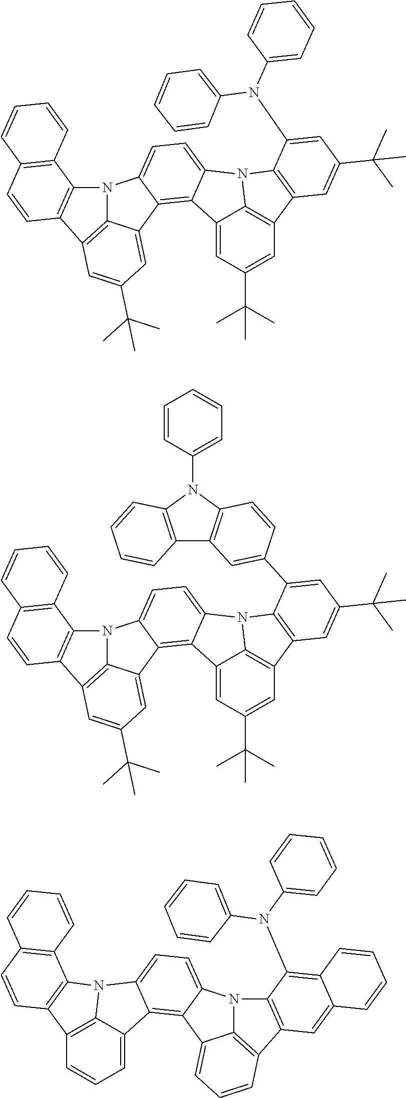 [SMILES: CC(C)(C)c1cc(N(c2ccccc2)c2ccccc2)c2c(c1)c1cc(C(C)(C)C)cc3c4c5c6cc(C(C)(C)C)cc7c8ccc9ccccc9c8n(c5ccc4n2c13)c76 ; CC(C)(C)c1cc(-c2ccc3c(c2)c2ccccc2n3-c2ccccc2)c2c(c1)c1cc(C(C)(C)C)cc3c4c5c6cc(C(C)(C)C)cc7c8ccc9ccccc9c8n(c5ccc4n2c13)c76 ; c1ccc(N(c2ccccc2)c2c3ccccc3cc3c4cccc5c6c7c8cccc9c%10ccc%11ccccc%11c%10n(c7ccc6n(c23)c45)c98)cc1]

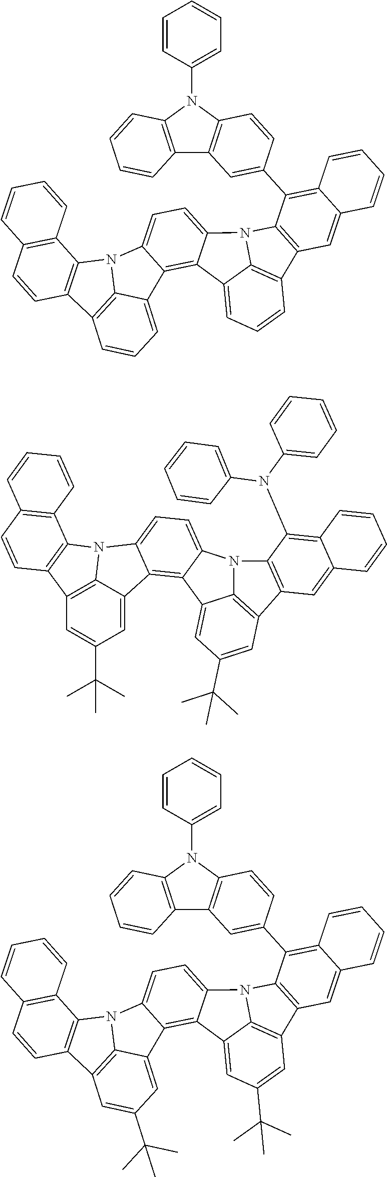

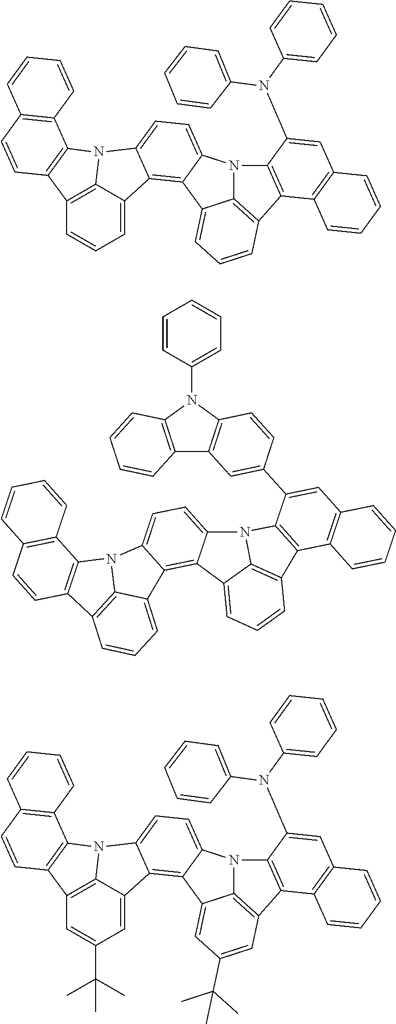

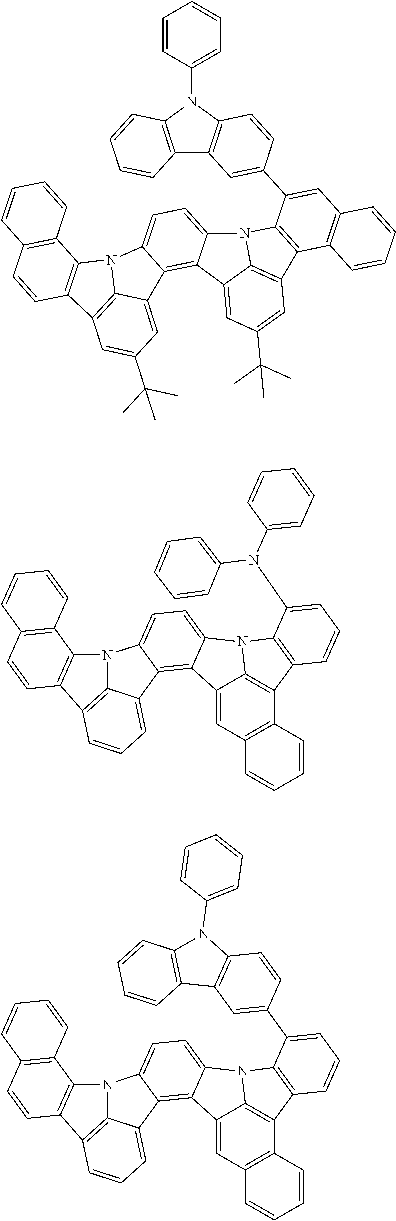 [SMILES: CC(C)(C)c1cc2c3c4c5cc(C(C)(C)C)cc6c7ccc8ccccc8c7n(c4ccc3n3c4c(-c7ccc8c(c7)c7ccccc7n8-c7ccccc7)cc7ccccc7c4c(c1)c23)c65 ; c1ccc(N(c2ccccc2)c2cccc3c4c5ccccc5cc5c6c7c8cccc9c%10ccc%11ccccc%11c%10n(c7ccc6n(c23)c54)c98)cc1 ; c1ccc(-n2c3ccccc3c3cc(-c4cccc5c6c7ccccc7cc7c8c9c%10cccc%11c%12ccc%13ccccc%13c%12n(c9ccc8n(c45)c76)c%11%10)ccc32)cc1]

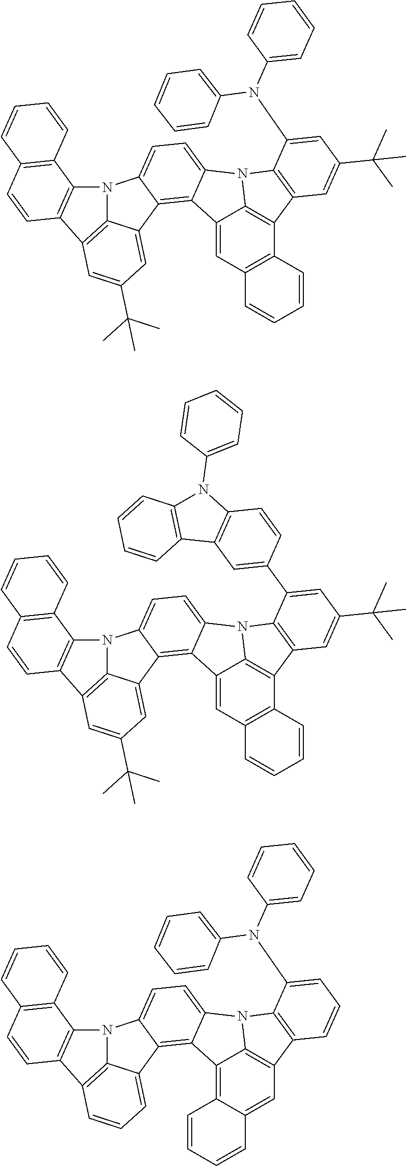 [SMILES: CC(C)(C)c1cc(N(c2ccccc2)c2ccccc2)c2c(c1)c1c3ccccc3cc3c4c5c6cc(C(C)(C)C)cc7c8ccc9ccccc9c8n(c5ccc4n2c31)c76 ; CC(C)(C)c1cc(-c2ccc3c(c2)c2ccccc2n3-c2ccccc2)c2c(c1)c1c3ccccc3cc3c4c5c6cc(C(C)(C)C)cc7c8ccc9ccccc9c8n(c5ccc4n2c31)c76 ; c1ccc(N(c2ccccc2)c2cccc3c4cc5ccccc5c5c6c7c8cccc9c%10ccc%11ccccc%11c%10n(c7ccc6n(c23)c45)c98)cc1]

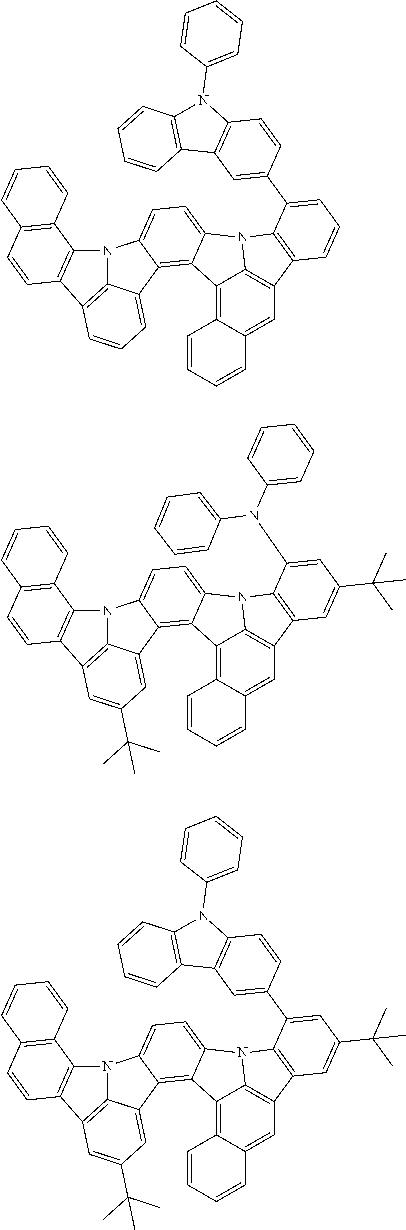 [SMILES: c1ccc(-n2c3ccccc3c3cc(-c4cccc5c6cc7ccccc7c7c8c9c%10cccc%11c%12ccc%13ccccc%13c%12n(c9ccc8n(c45)c67)c%11%10)ccc32)cc1 ; CC(C)(C)c1cc(N(c2ccccc2)c2ccccc2)c2c(c1)c1cc3ccccc3c3c4c5c6cc(C(C)(C)C)cc7c8ccc9ccccc9c8n(c5ccc4n2c13)c76 ; CC(C)(C)c1cc(-c2ccc3c(c2)c2ccccc2n3-c2ccccc2)c2c(c1)c1cc3ccccc3c3c4c5c6cc(C(C)(C)C)cc7c8ccc9ccccc9c8n(c5ccc4n2c13)c76]

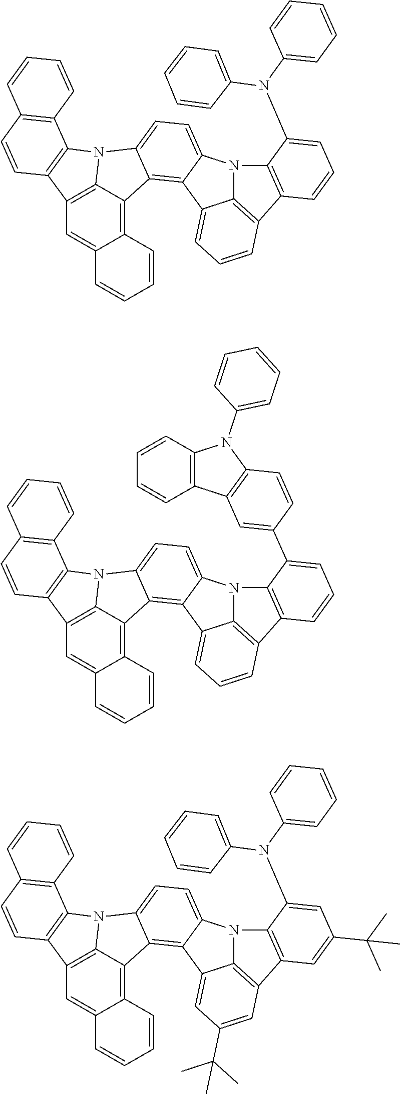 [SMILES: c1ccc(N(c2ccccc2)c2cccc3c4cccc5c6c7c8c9ccccc9cc9c%10ccc%11ccccc%11c%10n(c7ccc6n(c23)c45)c98)cc1 ; c1ccc(-n2c3ccccc3c3cc(-c4cccc5c6cccc7c8c9c%10c%11ccccc%11cc%11c%12ccc%13ccccc%13c%12n(c9ccc8n(c45)c67)c%11%10)ccc32)cc1 ; CC(C)(C)c1cc(N(c2ccccc2)c2ccccc2)c2c(c1)c1cc(C(C)(C)C)cc3c4c5c6c7ccccc7cc7c8ccc9ccccc9c8n(c5ccc4n2c13)c76]

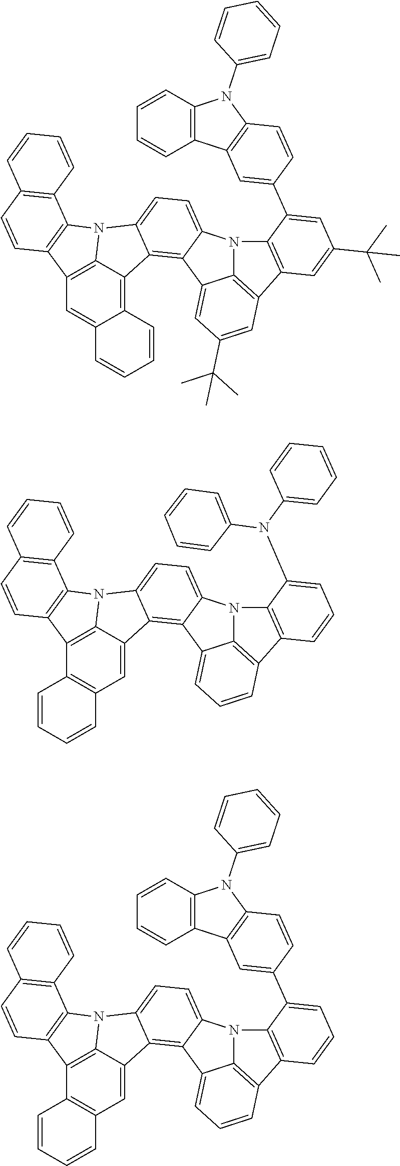 [SMILES: CC(C)(C)c1cc(-c2ccc3c(c2)c2ccccc2n3-c2ccccc2)c2c(c1)c1cc(C(C)(C)C)cc3c4c5c6c7ccccc7cc7c8ccc9ccccc9c8n(c5ccc4n2c13)c76 ; c1ccc(N(c2ccccc2)c2cccc3c4cccc5c6c7c8cc9ccccc9c9c%10ccc%11ccccc%11c%10n(c7ccc6n(c23)c45)c89)cc1 ; c1ccc(-n2c3ccccc3c3cc(-c4cccc5c6cccc7c8c9c%10cc%11ccccc%11c%11c%12ccc%13ccccc%13c%12n(c9ccc8n(c45)c67)c%10%11)ccc32)cc1]

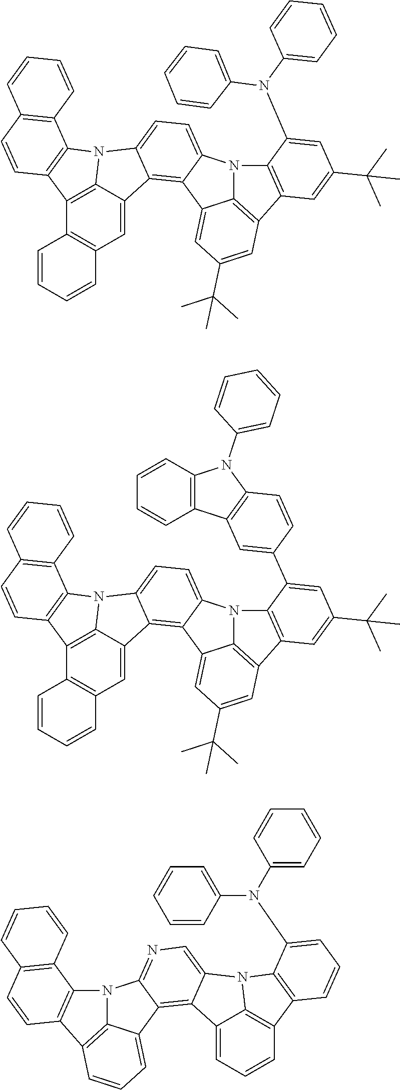 [SMILES: CC(C)(C)c1cc(N(c2ccccc2)c2ccccc2)c2c(c1)c1cc(C(C)(C)C)cc3c4c5c6cc7ccccc7c7c8ccc9ccccc9c8n(c5ccc4n2c13)c67 ; CC(C)(C)c1cc(-c2ccc3c(c2)c2ccccc2n3-c2ccccc2)c2c(c1)c1cc(C(C)(C)C)cc3c4c5c6cc7ccccc7c7c8ccc9ccccc9c8n(c5ccc4n2c13)c67 ; c1ccc(N(c2ccccc2)c2cccc3c4cccc5c6c7c8cccc9c%10ccc%11ccccc%11c%10n(c7ncc6n(c23)c45)c98)cc1]

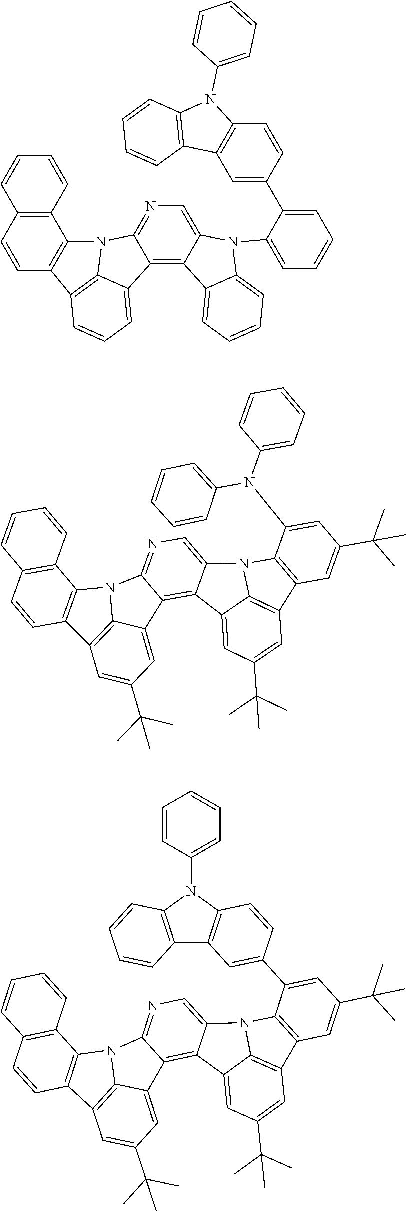 [SMILES: c1ccc(-n2c3ccccc3c3cc(-c4ccccc4-n4c5ccccc5c5c6c7cccc8c9ccc%10ccccc%10c9n(c6ncc54)c87)ccc32)cc1 ; CC(C)(C)c1cc(N(c2ccccc2)c2ccccc2)c2c(c1)c1cc(C(C)(C)C)cc3c4c5c6cc(C(C)(C)C)cc7c8ccc9ccccc9c8n(c5ncc4n2c13)c76 ; CC(C)(C)c1cc(-c2ccc3c(c2)c2ccccc2n3-c2ccccc2)c2c(c1)c1cc(C(C)(C)C)cc3c4c5c6cc(C(C)(C)C)cc7c8ccc9ccccc9c8n(c5ncc4n2c13)c76]

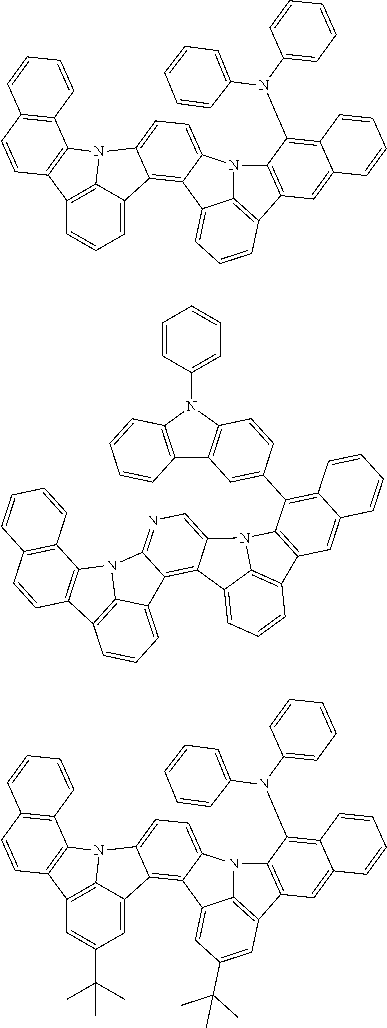 [SMILES: c1ccc(N(c2ccccc2)c2c3ccccc3cc3c4cccc5c6c7c8cccc9c%10ccc%11ccccc%11c%10n(c7ccc6n(c23)c45)c98)cc1 ; c1ccc(-n2c3ccccc3c3cc(-c4c5ccccc5cc5c6cccc7c8c9c%10cccc%11c%12ccc%13ccccc%13c%12n(c9ncc8n(c45)c67)c%11%10)ccc32)cc1 ; CC(C)(C)c1cc2c3cc4ccccc4c(N(c4ccccc4)c4ccccc4)c3n3c4ccc5c(c6cc(C(C)(C)C)cc7c8ccc9ccccc9c8n5c76)c4c(c1)c23]

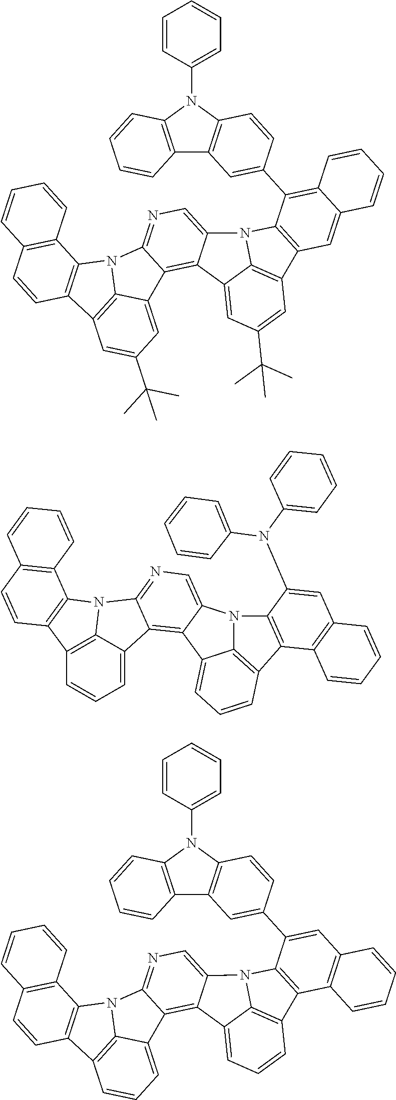

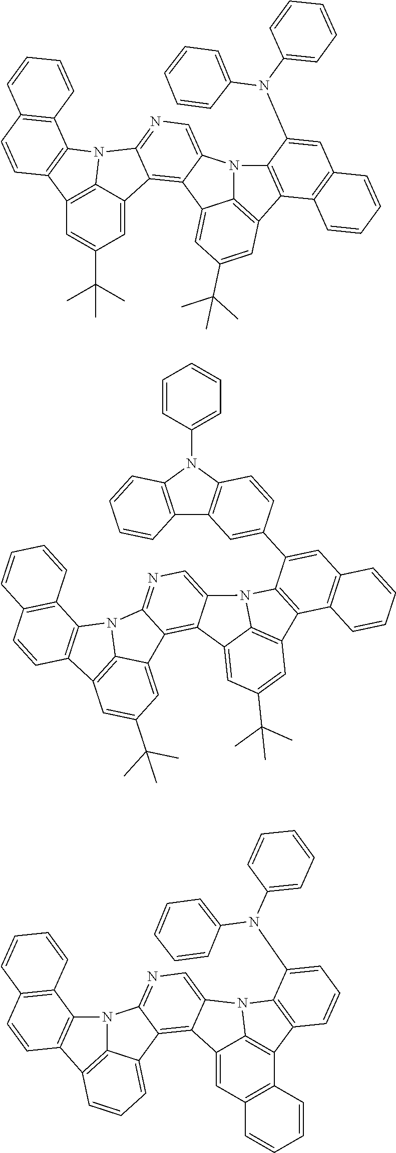

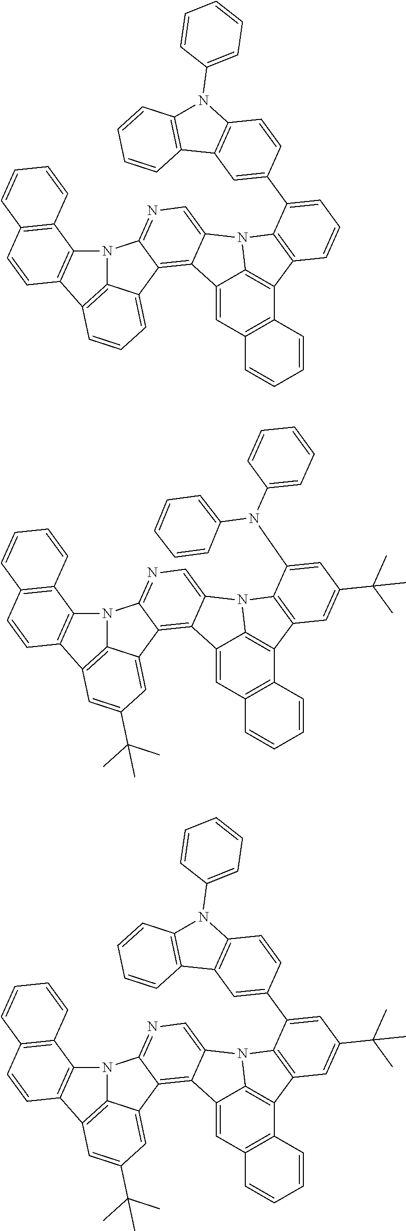

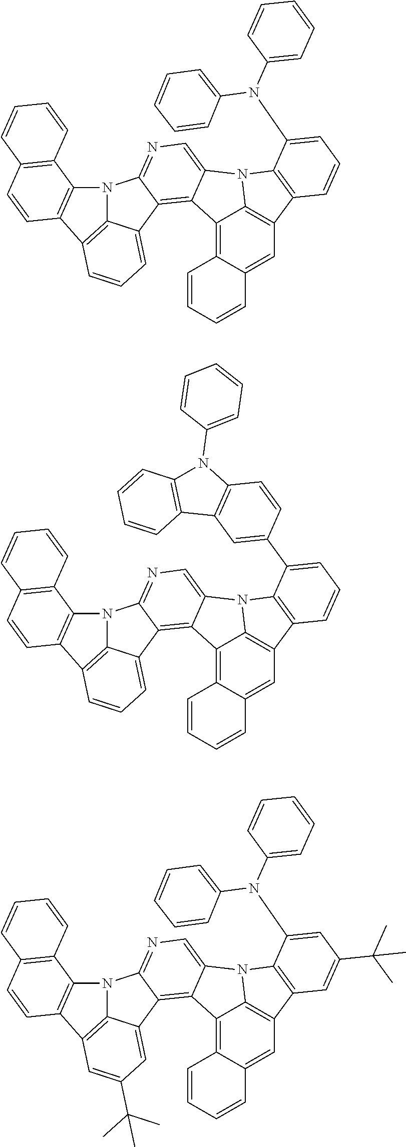

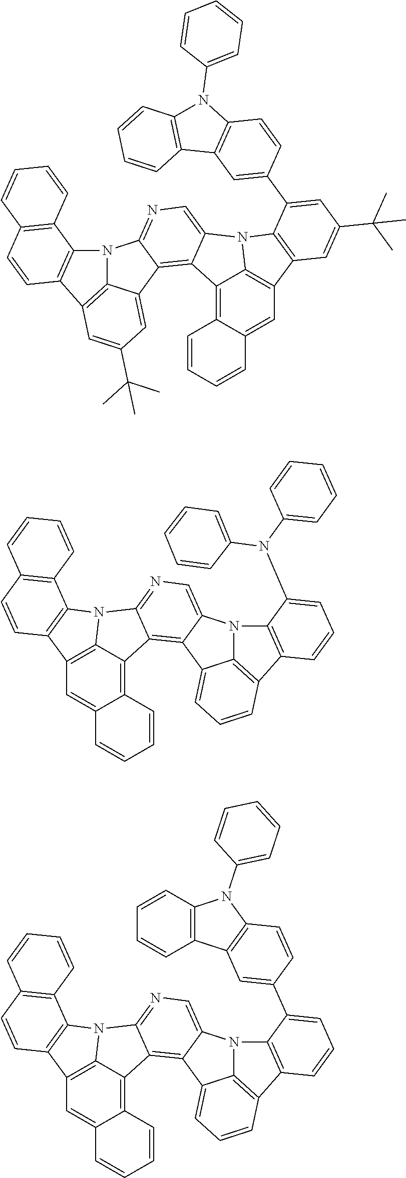 [SMILES: CC(C)(C)c1cc(-c2ccc3c(c2)c2ccccc2n3-c2ccccc2)c2c(c1)c1cc3ccccc3c3c4c5c6cc(C(C)(C)C)cc7c8ccc9ccccc9c8n(c5ncc4n2c13)c76 ; c1ccc(N(c2ccccc2)c2cccc3c4cccc5c6c7c8c9ccccc9cc9c%10ccc%11ccccc%11c%10n(c7ncc6n(c23)c45)c98)cc1 ; c1ccc(-n2c3ccccc3c3cc(-c4cccc5c6cccc7c8c9c%10c%11ccccc%11cc%11c%12ccc%13ccccc%13c%12n(c9ncc8n(c45)c67)c%11%10)ccc32)cc1]

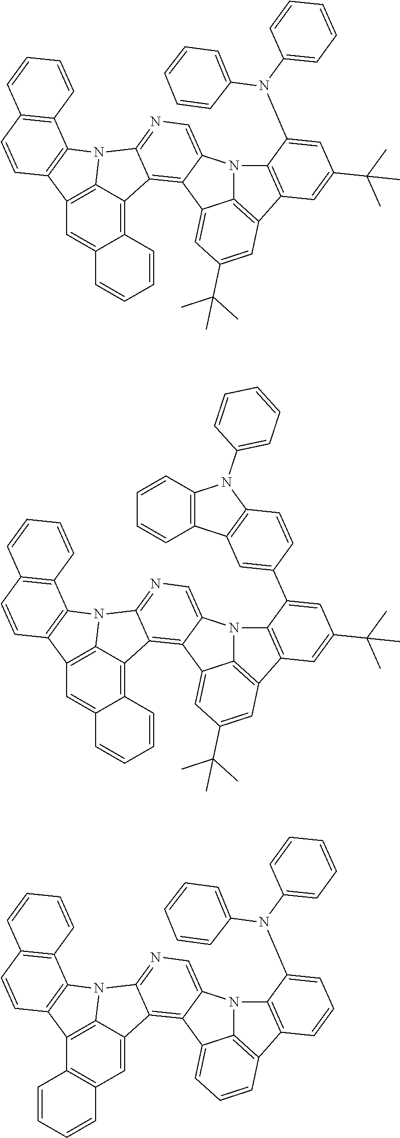 [SMILES: CC(C)(C)c1cc(N(c2ccccc2)c2ccccc2)c2c(c1)c1cc(C(C)(C)C)cc3c4c5c6c7ccccc7cc7c8ccc9ccccc9c8n(c5ncc4n2c13)c76 ; CC(C)(C)c1cc(-c2ccc3c(c2)c2ccccc2n3-c2ccccc2)c2c(c1)c1cc(C(C)(C)C)cc3c4c5c6c7ccccc7cc7c8ccc9ccccc9c8n(c5ncc4n2c13)c76 ; c1ccc(N(c2ccccc2)c2cccc3c4cccc5c6c7c8cc9ccccc9c9c%10ccc%11ccccc%11c%10n(c7ncc6n(c23)c45)c89)cc1]

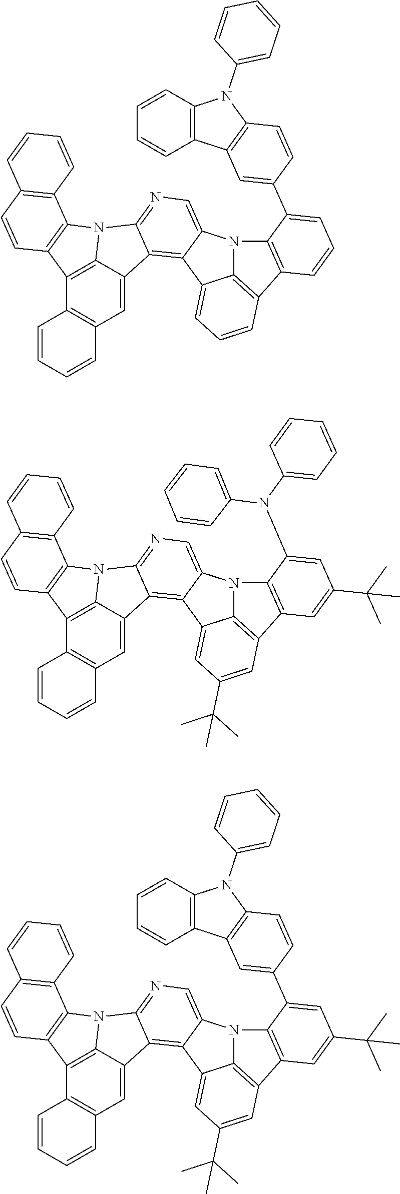 [SMILES: c1ccc(-n2c3ccccc3c3cc(-c4cccc5c6cccc7c8c9c%10cc%11ccccc%11c%11c%12ccc%13ccccc%13c%12n(c9ncc8n(c45)c67)c%10%11)ccc32)cc1 ; CC(C)(C)c1cc(N(c2ccccc2)c2ccccc2)c2c(c1)c1cc(C(C)(C)C)cc3c4c5c6cc7ccccc7c7c8ccc9ccccc9c8n(c5ncc4n2c13)c67 ; CC(C)(C)c1cc(-c2ccc3c(c2)c2ccccc2n3-c2ccccc2)c2c(c1)c1cc(C(C)(C)C)cc3c4c5c6cc7ccccc7c7c8ccc9ccccc9c8n(c5ncc4n2c13)c67]

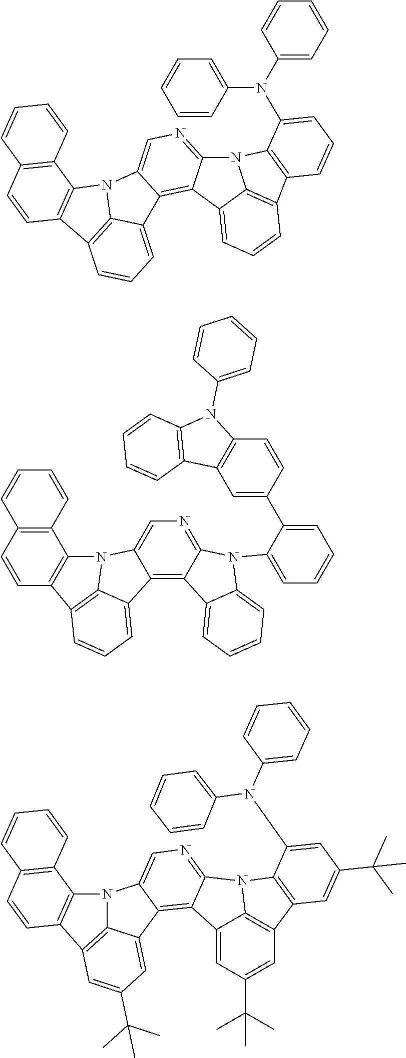 [SMILES: c1ccc(N(c2ccccc2)c2cccc3c4cccc5c6c7c8cccc9c%10ccc%11ccccc%11c%10n(c7cnc6n(c23)c45)c98)cc1 ; c1ccc(-n2c3ccccc3c3cc(-c4ccccc4-n4c5ccccc5c5c6c7cccc8c9ccc%10ccccc%10c9n(c6cnc54)c87)ccc32)cc1 ; CC(C)(C)c1cc(N(c2ccccc2)c2ccccc2)c2c(c1)c1cc(C(C)(C)C)cc3c4c5c6cc(C(C)(C)C)cc7c8ccc9ccccc9c8n(c5cnc4n2c13)c76]

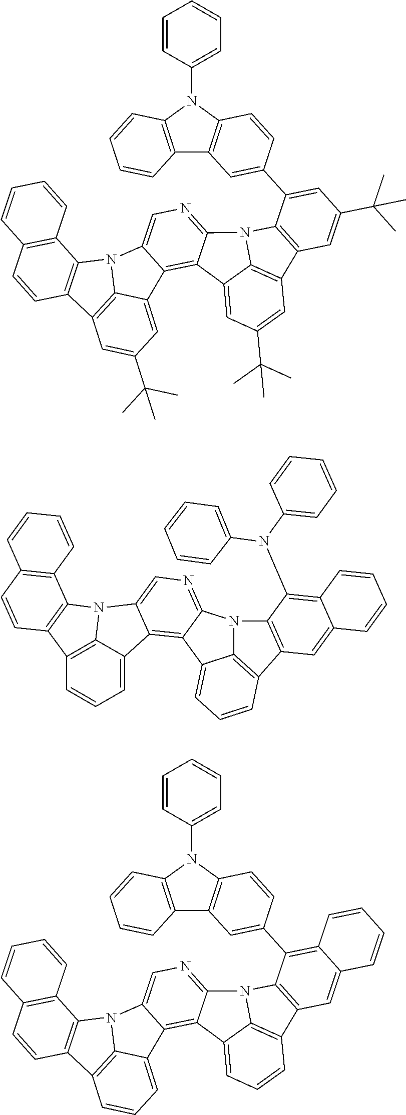 [SMILES: CC(C)(C)c1cc(-c2ccc3c(c2)c2ccccc2n3-c2ccccc2)c2c(c1)c1cc(C(C)(C)C)cc3c4c5c6cc(C(C)(C)C)cc7c8ccc9ccccc9c8n(c5cnc4n2c13)c76 ; c1ccc(N(c2ccccc2)c2c3ccccc3cc3c4cccc5c6c7c8cccc9c%10ccc%11ccccc%11c%10n(c7cnc6n(c23)c45)c98)cc1 ; c1ccc(-n2c3ccccc3c3cc(-c4c5ccccc5cc5c6cccc7c8c9c%10cccc%11c%12ccc%13ccccc%13c%12n(c9cnc8n(c45)c67)c%11%10)ccc32)cc1]

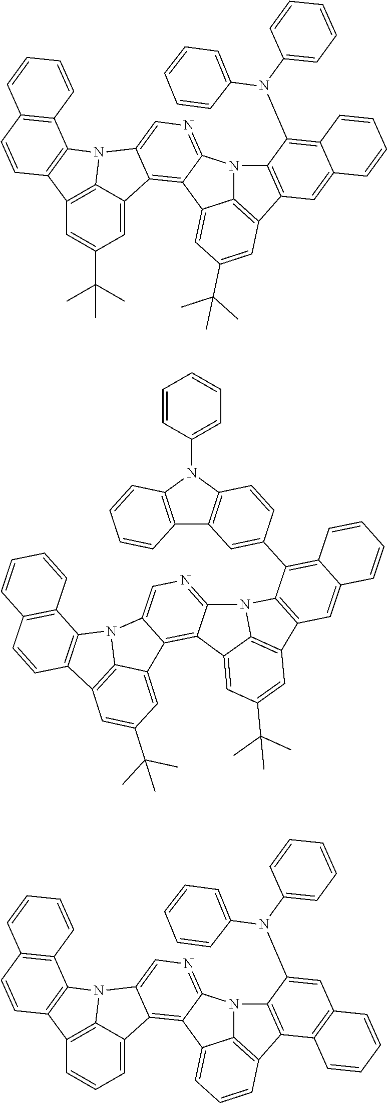

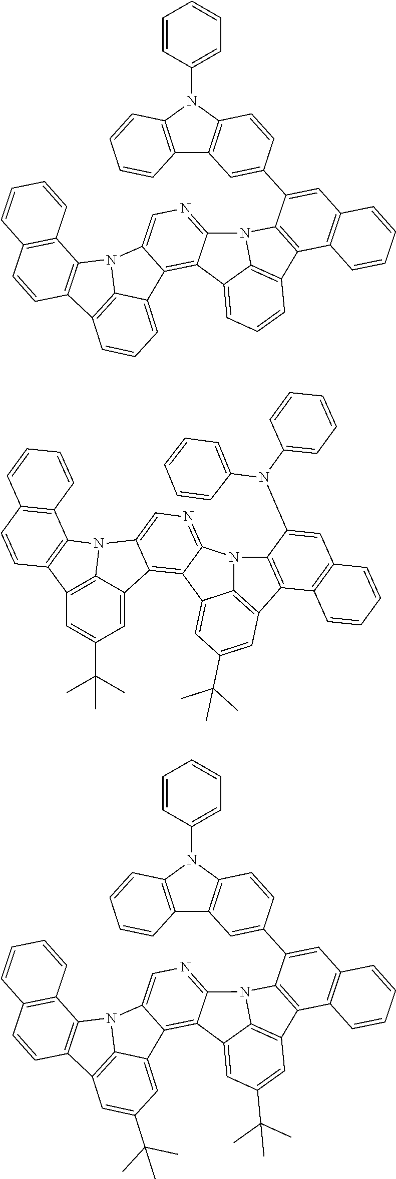

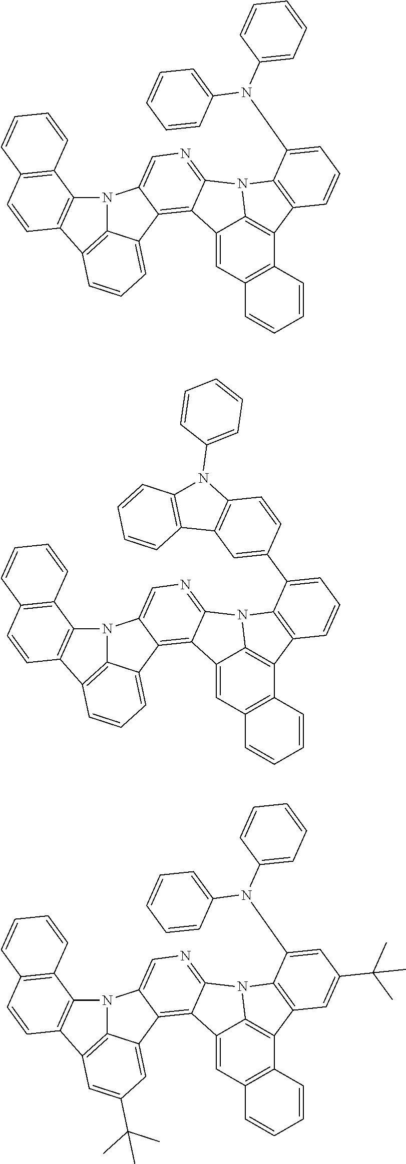

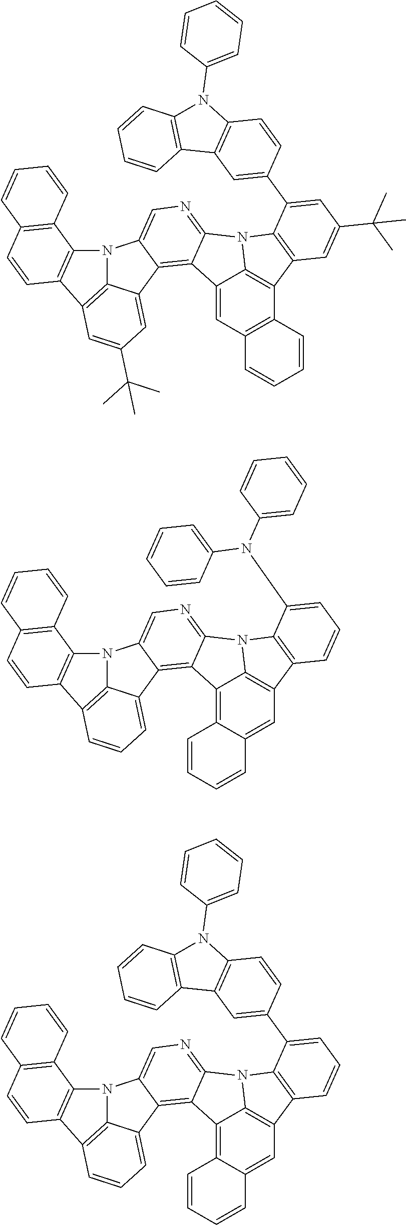 [SMILES: CC(C)(C)c1cc(-c2ccc3c(c2)c2ccccc2n3-c2ccccc2)c2c(c1)c1c3ccccc3cc3c4c5c6cc(C(C)(C)C)cc7c8ccc9ccccc9c8n(c5cnc4n2c31)c76 ; c1ccc(N(c2ccccc2)c2cccc3c4cc5ccccc5c5c6c7c8cccc9c%10ccc%11ccccc%11c%10n(c7cnc6n(c23)c45)c98)cc1 ; c1ccc(-n2c3ccccc3c3cc(-c4cccc5c6cc7ccccc7c7c8c9c%10cccc%11c%12ccc%13ccccc%13c%12n(c9cnc8n(c45)c67)c%11%10)ccc32)cc1]

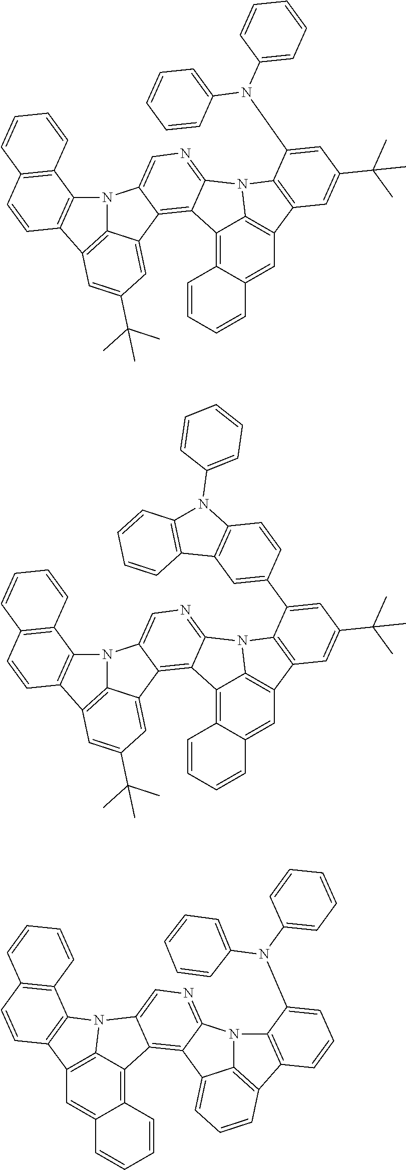 [SMILES: CC(C)(C)c1cc(N(c2ccccc2)c2ccccc2)c2c(c1)c1cc3ccccc3c3c4c5c6cc(C(C)(C)C)cc7c8ccc9ccccc9c8n(c5cnc4n2c13)c76 ; CC(C)(C)c1cc(-c2ccc3c(c2)c2ccccc2n3-c2ccccc2)c2c(c1)c1cc3ccccc3c3c4c5c6cc(C(C)(C)C)cc7c8ccc9ccccc9c8n(c5cnc4n2c13)c76 ; c1ccc(N(c2ccccc2)c2cccc3c4cccc5c6c7c8c9ccccc9cc9c%10ccc%11ccccc%11c%10n(c7cnc6n(c23)c45)c98)cc1]

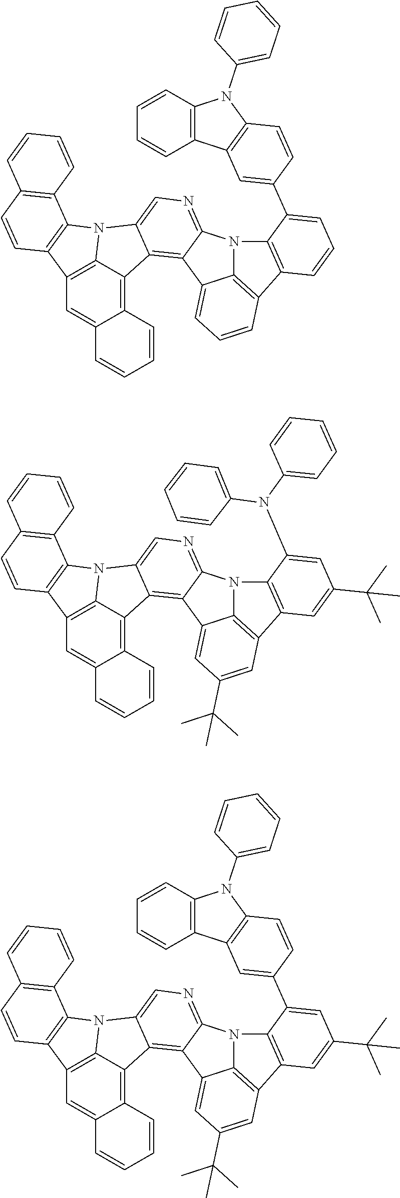 [SMILES: c1ccc(-n2c3ccccc3c3cc(-c4cccc5c6cccc7c8c9c%10c%11ccccc%11cc%11c%12ccc%13ccccc%13c%12n(c9cnc8n(c45)c67)c%11%10)ccc32)cc1 ; CC(C)(C)c1cc(N(c2ccccc2)c2ccccc2)c2c(c1)c1cc(C(C)(C)C)cc3c4c5c6c7ccccc7cc7c8ccc9ccccc9c8n(c5cnc4n2c13)c76 ; CC(C)(C)c1cc(-c2ccc3c(c2)c2ccccc2n3-c2ccccc2)c2c(c1)c1cc(C(C)(C)C)cc3c4c5c6c7ccccc7cc7c8ccc9ccccc9c8n(c5cnc4n2c13)c76]

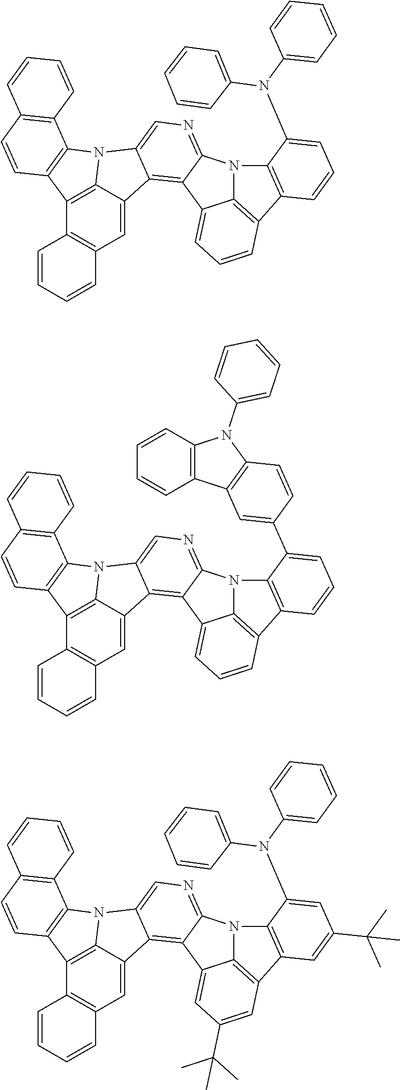 [SMILES: c1ccc(N(c2ccccc2)c2cccc3c4cccc5c6c7c8cc9ccccc9c9c%10ccc%11ccccc%11c%10n(c7cnc6n(c23)c45)c89)cc1 ; c1ccc(-n2c3ccccc3c3cc(-c4cccc5c6cccc7c8c9c%10cc%11ccccc%11c%11c%12ccc%13ccccc%13c%12n(c9cnc8n(c45)c67)c%10%11)ccc32)cc1 ; CC(C)(C)c1cc(N(c2ccccc2)c2ccccc2)c2c(c1)c1cc(C(C)(C)C)cc3c4c5c6cc7ccccc7c7c8ccc9ccccc9c8n(c5cnc4n2c13)c67]

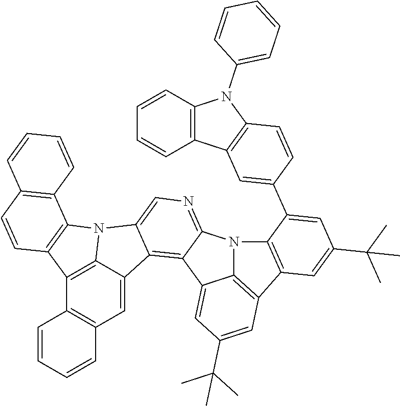 [SMILES: CC(C)(C)c1cc(-c2ccc3c(c2)c2ccccc2n3-c2ccccc2)c2c(c1)c1cc(C(C)(C)C)cc3c4c5c6cc7ccccc7c7c8ccc9ccccc9c8n(c5cnc4n2c13)c67]

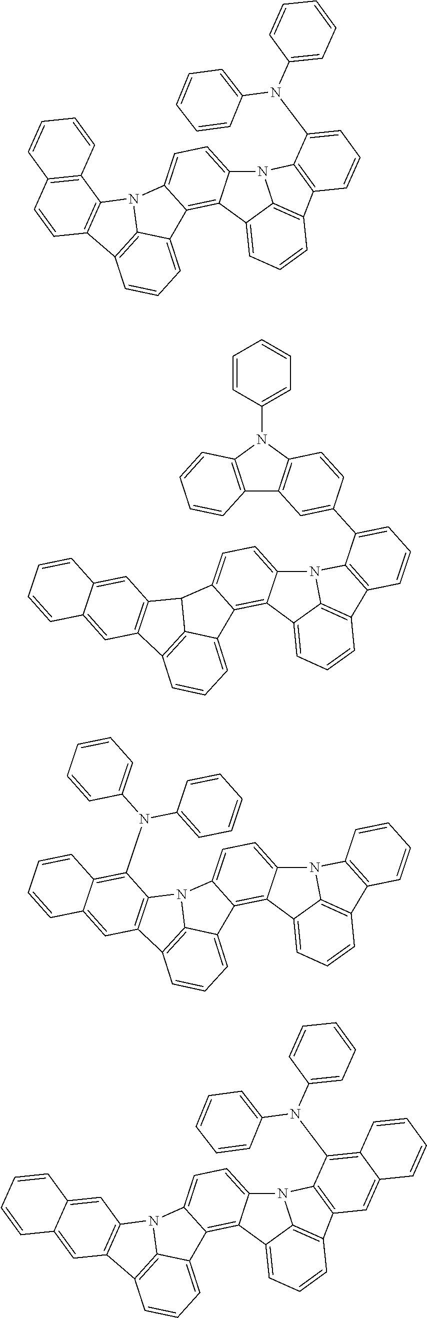 [SMILES: c1ccc(N(c2ccccc2)c2cccc3c4cccc5c6c7c8cccc9c%10ccc%11ccccc%11c%10n(c7ccc6n(c23)c45)c98)cc1 ; c1ccc(-n2c3ccccc3c3cc(-c4cccc5c6cccc7c8c9c(ccc8n(c45)c67)C4c5cc6ccccc6cc5-c5cccc-9c54)ccc32)cc1 ; c1ccc(N(c2ccccc2)c2c3ccccc3cc3c4cccc5c6c7c8cccc9c%10ccccc%10n(c7ccc6n(c23)c45)c98)cc1 ; c1ccc(N(c2ccccc2)c2c3ccccc3cc3c4cccc5c6c7c8cccc9c%10cc%11ccccc%11cc%10n(c7ccc6n(c23)c45)c98)cc1]

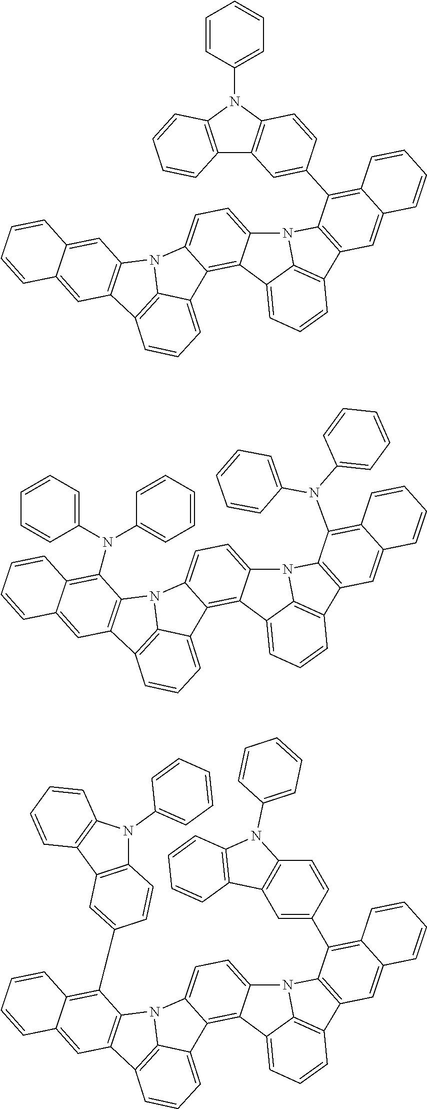 [SMILES: c1ccc(-n2c3ccccc3c3cc(-c4c5ccccc5cc5c6cccc7c8c9c%10cccc%11c%12cc%13ccccc%13cc%12n(c9ccc8n(c45)c67)c%11%10)ccc32)cc1 ; c1ccc(N(c2ccccc2)c2c3ccccc3cc3c4cccc5c6c7c8cccc9c%10cc%11ccccc%11c(N(c%11ccccc%11)c%11ccccc%11)c%10n(c7ccc6n(c23)c45)c98)cc1 ; c1ccc(-n2c3ccccc3c3cc(-c4c5ccccc5cc5c6cccc7c8c9c%10cccc%11c%12cc%13ccccc%13c(-c%13ccc%14c(c%13)c%13ccccc%13n%14-c%13ccccc%13)c%12n(c9ccc8n(c45)c67)c%11%10)ccc32)cc1]

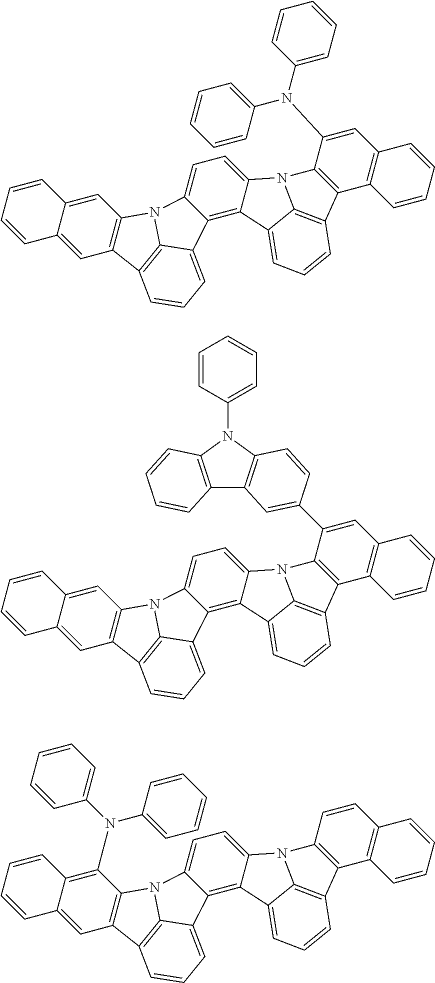 [SMILES: c1ccc(N(c2ccccc2)c2cc3ccccc3c3c4cccc5c6c7c8cccc9c%10cc%11ccccc%11cc%10n(c7ccc6n(c23)c54)c98)cc1 ; c1ccc(-n2c3ccccc3c3cc(-c4cc5ccccc5c5c6cccc7c8c9c%10cccc%11c%12cc%13ccccc%13cc%12n(c9ccc8n(c45)c76)c%11%10)ccc32)cc1 ; c1ccc(N(c2ccccc2)c2c3ccccc3cc3c4cccc5c6c7c8cccc9c%10c%11ccccc%11ccc%10n(c7ccc6n(c23)c45)c98)cc1]

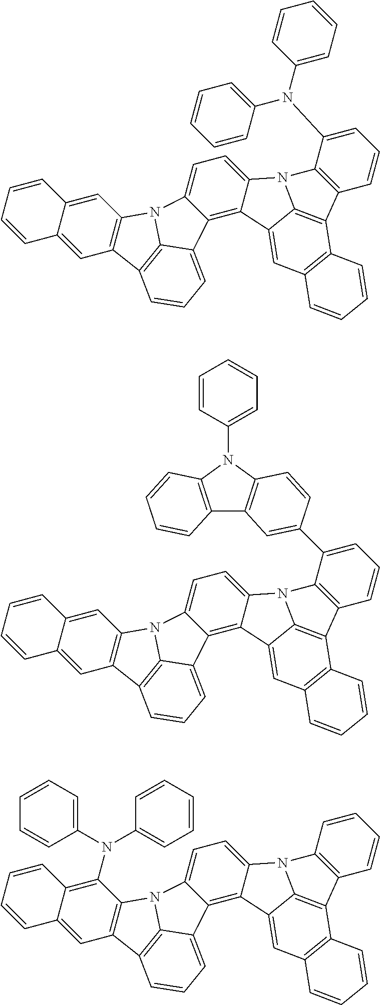 [SMILES: c1ccc(N(c2ccccc2)c2cccc3c4c5ccccc5cc5c6c7c8cccc9c%10cc%11ccccc%11cc%10n(c7ccc6n(c23)c54)c98)cc1 ; c1ccc(-n2c3ccccc3c3cc(-c4cccc5c6c7ccccc7cc7c8c9c%10cccc%11c%12cc%13ccccc%13cc%12n(c9ccc8n(c45)c76)c%11%10)ccc32)cc1 ; c1ccc(N(c2ccccc2)c2c3ccccc3cc3c4cccc5c6c7c8cc9ccccc9c9c%10ccccc%10n(c7ccc6n(c23)c45)c89)cc1]

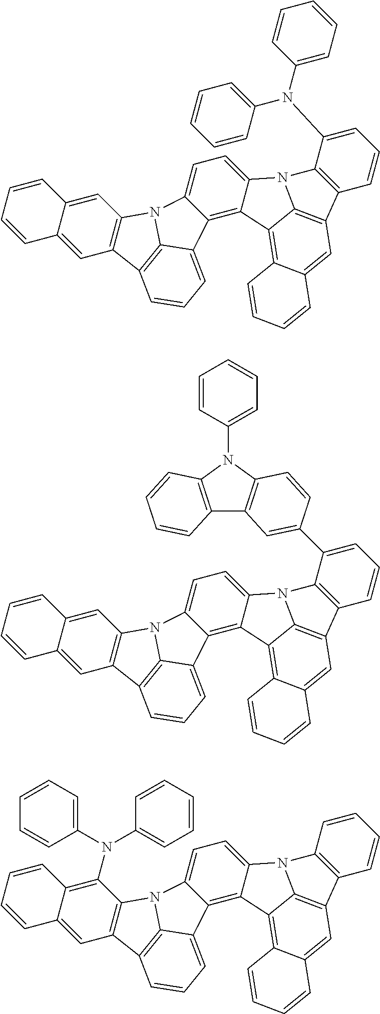 [SMILES: c1ccc(N(c2ccccc2)c2cccc3c4cc5ccccc5c5c6c7c8cccc9c%10cc%11ccccc%11cc%10n(c7ccc6n(c23)c45)c98)cc1 ; c1ccc(-n2c3ccccc3c3cc(-c4cccc5c6cc7ccccc7c7c8c9c%10cccc%11c%12cc%13ccccc%13cc%12n(c9ccc8n(c45)c67)c%11%10)ccc32)cc1 ; c1ccc(N(c2ccccc2)c2c3ccccc3cc3c4cccc5c6c7c8c9ccccc9cc9c%10ccccc%10n(c7ccc6n(c23)c45)c98)cc1]

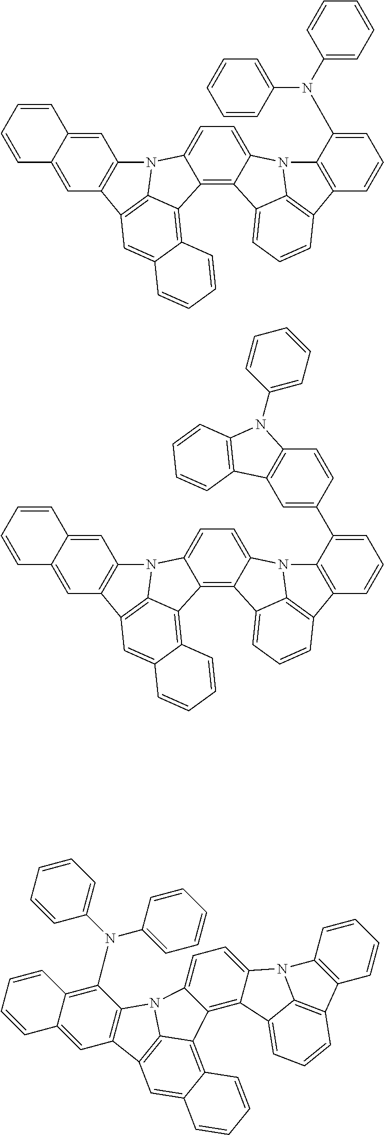 [SMILES: c1ccc(N(c2ccccc2)c2cccc3c4cccc5c6c7c8c9ccccc9cc9c%10cc%11ccccc%11cc%10n(c7ccc6n(c23)c45)c98)cc1 ; c1ccc(-n2c3ccccc3c3cc(-c4cccc5c6cccc7c8c9c%10c%11ccccc%11cc%11c%12cc%13ccccc%13cc%12n(c9ccc8n(c45)c67)c%11%10)ccc32)cc1 ; c1ccc(N(c2ccccc2)c2c3ccccc3cc3c4cc5ccccc5c5c6c7c8cccc9c%10ccccc%10n(c7ccc6n(c23)c45)c98)cc1]

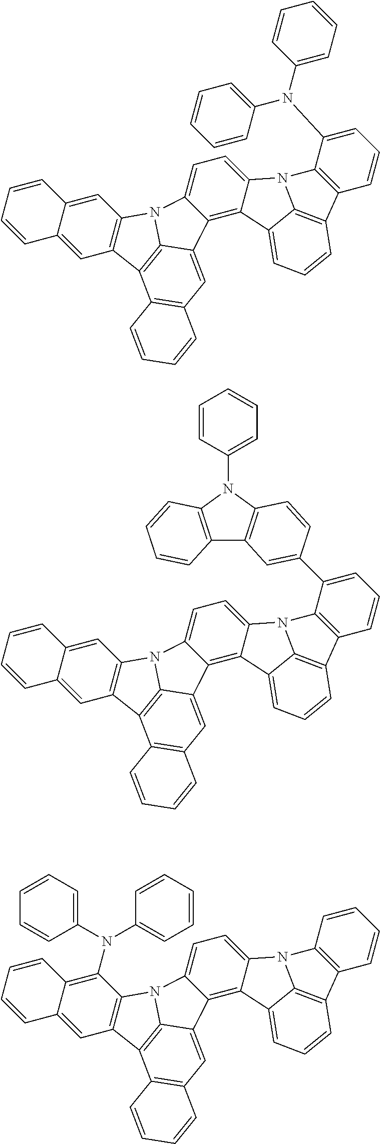 [SMILES: c1ccc(N(c2ccccc2)c2cccc3c4cccc5c6c7c8cc9ccccc9c9c%10cc%11ccccc%11cc%10n(c7ccc6n(c23)c45)c89)cc1 ; c1ccc(-n2c3ccccc3c3cc(-c4cccc5c6cccc7c8c9c%10cc%11ccccc%11c%11c%12cc%13ccccc%13cc%12n(c9ccc8n(c45)c67)c%10%11)ccc32)cc1 ; c1ccc(N(c2ccccc2)c2c3ccccc3cc3c4c5ccccc5cc5c6c7c8cccc9c%10ccccc%10n(c7ccc6n(c23)c54)c98)cc1]

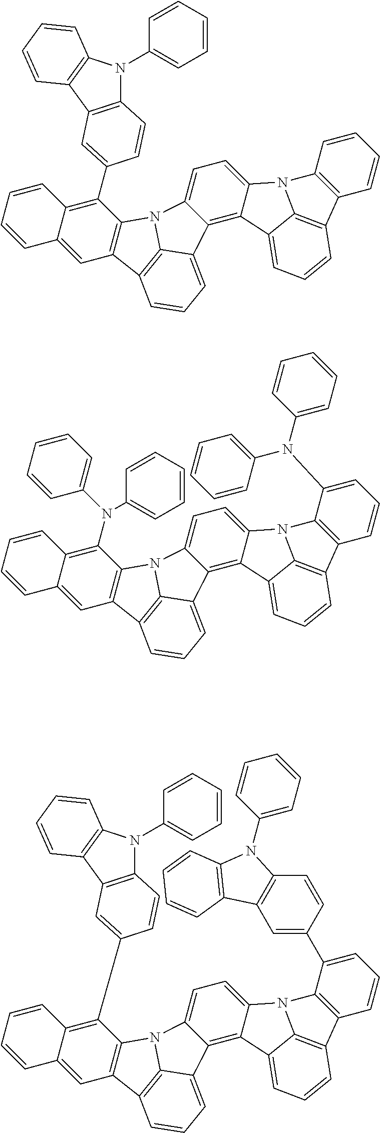 [SMILES: c1ccc(-n2c3ccccc3c3cc(-c4c5ccccc5cc5c6cccc7c8c9c%10cccc%11c%12ccccc%12n(c9ccc8n(c45)c67)c%11%10)ccc32)cc1 ; c1ccc(N(c2ccccc2)c2cccc3c4cccc5c6c7c8cccc9c%10cc%11ccccc%11c(N(c%11ccccc%11)c%11ccccc%11)c%10n(c7ccc6n(c23)c45)c98)cc1 ; c1ccc(-n2c3ccccc3c3cc(-c4cccc5c6cccc7c8c9c%10cccc%11c%12cc%13ccccc%13c(-c%13ccc%14c(c%13)c%13ccccc%13n%14-c%13ccccc%13)c%12n(c9ccc8n(c45)c67)c%11%10)ccc32)cc1]

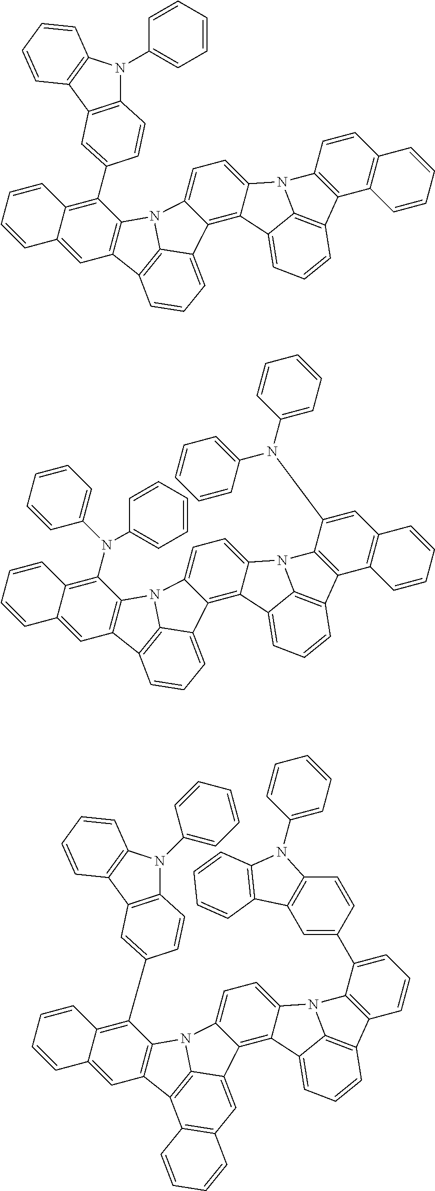 [SMILES: c1ccc(-n2c3ccccc3c3cc(-c4c5ccccc5cc5c6cccc7c8c9c%10cccc%11c%12c%13ccccc%13ccc%12n(c9ccc8n(c45)c67)c%11%10)ccc32)cc1 ; c1ccc(N(c2ccccc2)c2c3ccccc3cc3c4cccc5c6c7c8cccc9c%10c%11ccccc%11cc(N(c%11ccccc%11)c%11ccccc%11)c%10n(c7ccc6n(c23)c45)c89)cc1 ; c1ccc(-n2c3ccccc3c3cc(-c4cccc5c6cccc7c8c9c%10cc%11ccccc%11c%11c%12cc%13ccccc%13c(-c%13ccc%14c(c%13)c%13ccccc%13n%14-c%13ccccc%13)c%12n(c9ccc8n(c45)c67)c%10%11)ccc32)cc1]

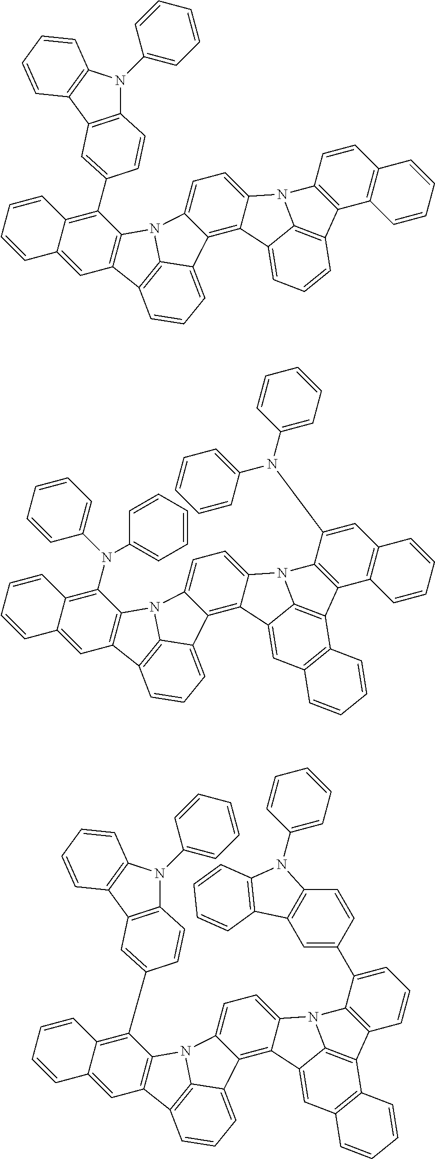 [SMILES: c1ccc(-n2c3ccccc3c3cc(-c4c5ccccc5cc5c6cccc7c8c9c%10cccc%11c%12c%13ccccc%13ccc%12n(c9ccc8n(c45)c67)c%11%10)ccc32)cc1 ; c1ccc(N(c2ccccc2)c2c3ccccc3cc3c4cccc5c6c7c8cc9ccccc9c9c%10c%11ccccc%11cc(N(c%11ccccc%11)c%11ccccc%11)c%10n(c7ccc6n(c23)c45)c89)cc1 ; c1ccc(-n2c3ccccc3c3cc(-c4c5ccccc5cc5c6cccc7c8c9c%10cc%11ccccc%11c%11c%12cccc(-c%13ccc%14c(c%13)c%13ccccc%13n%14-c%13ccccc%13)c%12n(c9ccc8n(c45)c67)c%10%11)ccc32)cc1]

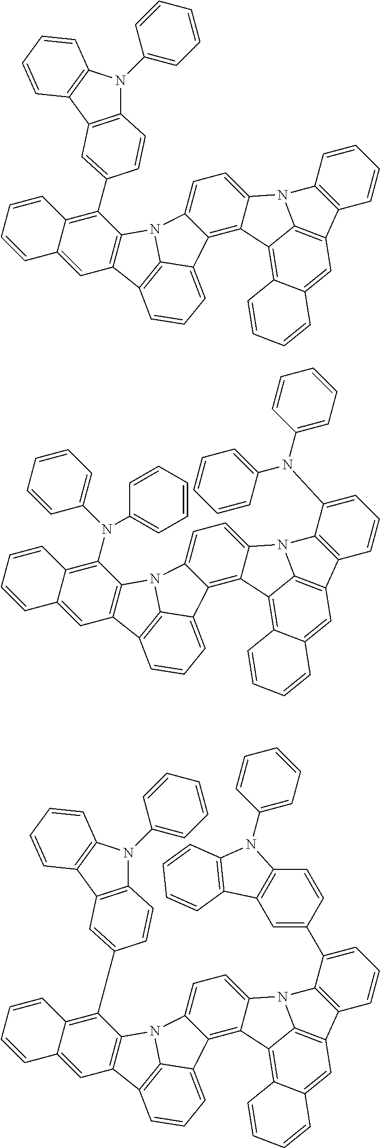

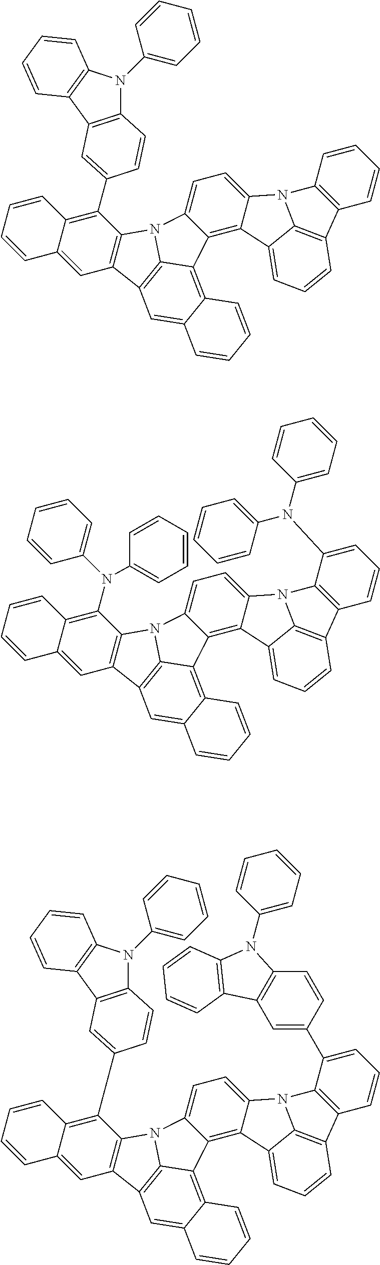 [SMILES: c1ccc(-n2c3ccccc3c3cc(-c4c5ccccc5cc5c6cc7ccccc7c7c8c9c%10cccc%11c%12ccccc%12n(c9ccc8n(c45)c67)c%11%10)ccc32)cc1 ; c1ccc(N(c2ccccc2)c2cccc3c4cccc5c6c7c8c9ccccc9cc9c%10cc%11ccccc%11c(N(c%11ccccc%11)c%11ccccc%11)c%10n(c7ccc6n(c23)c45)c98)cc1 ; c1ccc(-n2c3ccccc3c3cc(-c4cccc5c6cccc7c8c9c%10c%11ccccc%11cc%11c%12cc%13ccccc%13c(-c%13ccc%14c(c%13)c%13ccccc%13n%14-c%13ccccc%13)c%12n(c9ccc8n(c45)c67)c%11%10)ccc32)cc1]

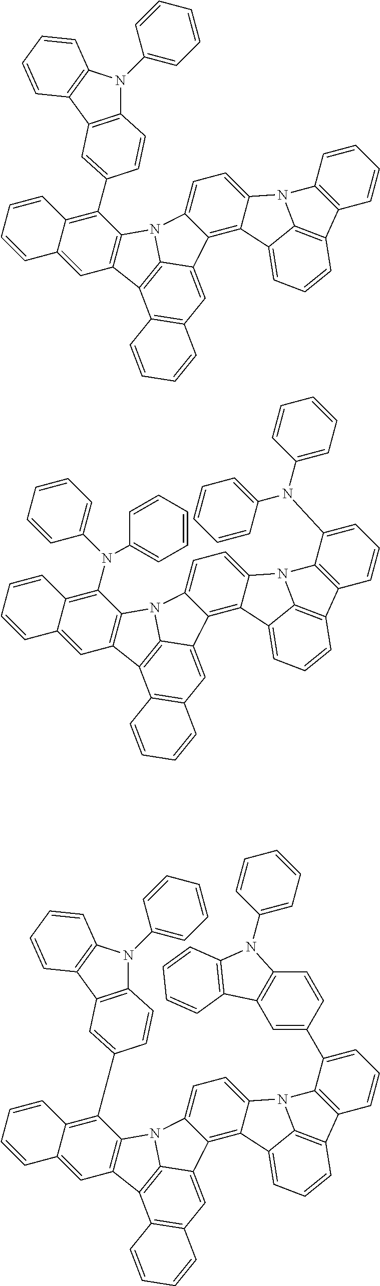 [SMILES: c1ccc(-n2c3ccccc3c3cc(-c4c5ccccc5cc5c6c7ccccc7cc7c8c9c%10cccc%11c%12ccccc%12n(c9ccc8n(c45)c76)c%11%10)ccc32)cc1 ; c1ccc(N(c2ccccc2)c2cccc3c4cccc5c6c7c8cc9ccccc9c9c%10cc%11ccccc%11c(N(c%11ccccc%11)c%11ccccc%11)c%10n(c7ccc6n(c23)c45)c89)cc1 ; c1ccc(-n2c3ccccc3c3cc(-c4cccc5c6cccc7c8c9c%10cc%11ccccc%11c%11c%12cc%13ccccc%13c(-c%13ccc%14c(c%13)c%13ccccc%13n%14-c%13ccccc%13)c%12n(c9ccc8n(c45)c67)c%10%11)ccc32)cc1]

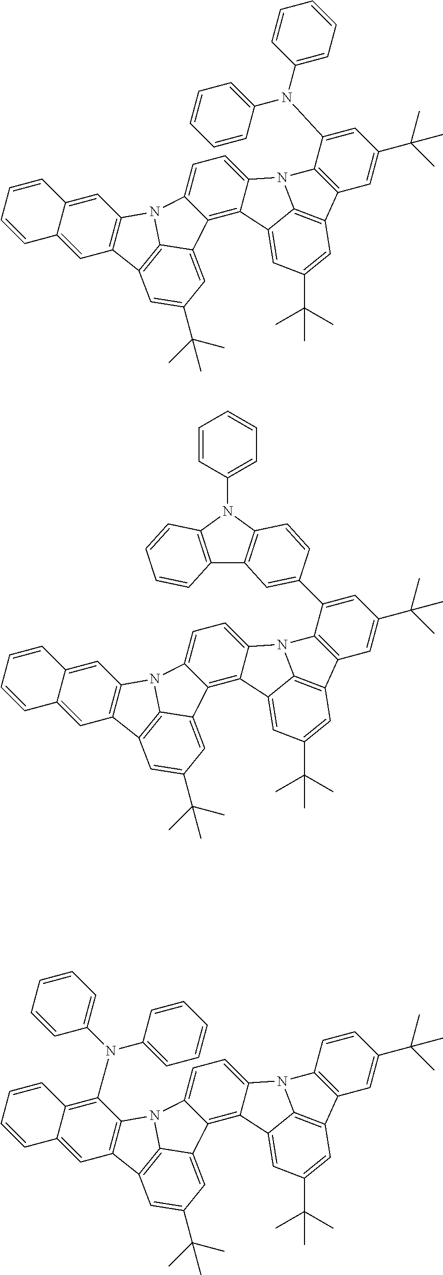 [SMILES: CC(C)(C)c1cc(N(c2ccccc2)c2ccccc2)c2c(c1)c1cc(C(C)(C)C)cc3c4c5c6cc(C(C)(C)C)cc7c8cc9ccccc9cc8n(c5ccc4n2c13)c76 ; CC(C)(C)c1cc(-c2ccc3c(c2)c2ccccc2n3-c2ccccc2)c2c(c1)c1cc(C(C)(C)C)cc3c4c5c6cc(C(C)(C)C)cc7c8cc9ccccc9cc8n(c5ccc4n2c13)c76 ; CC(C)(C)c1ccc2c(c1)c1cc(C(C)(C)C)cc3c4c5c6cc(C(C)(C)C)cc7c8cc9ccccc9c(N(c9ccccc9)c9ccccc9)c8n(c5ccc4n2c13)c76]

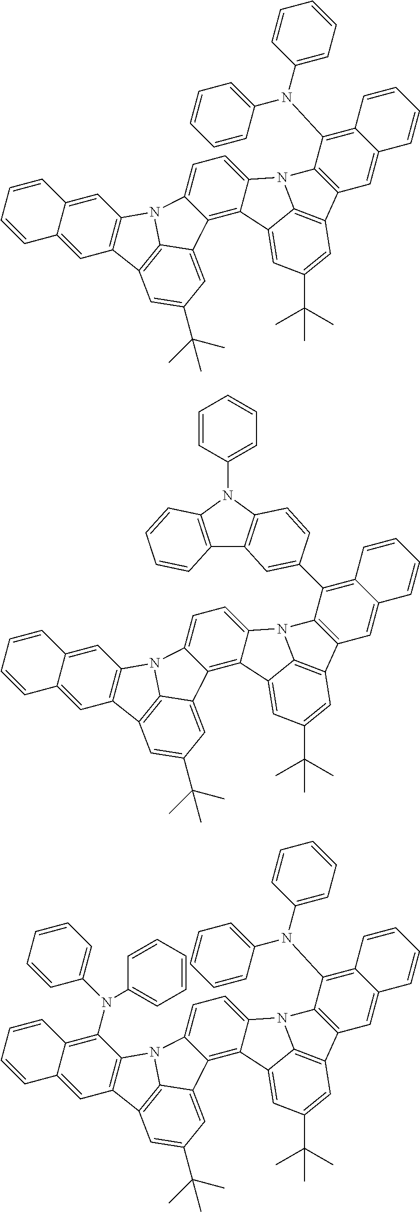

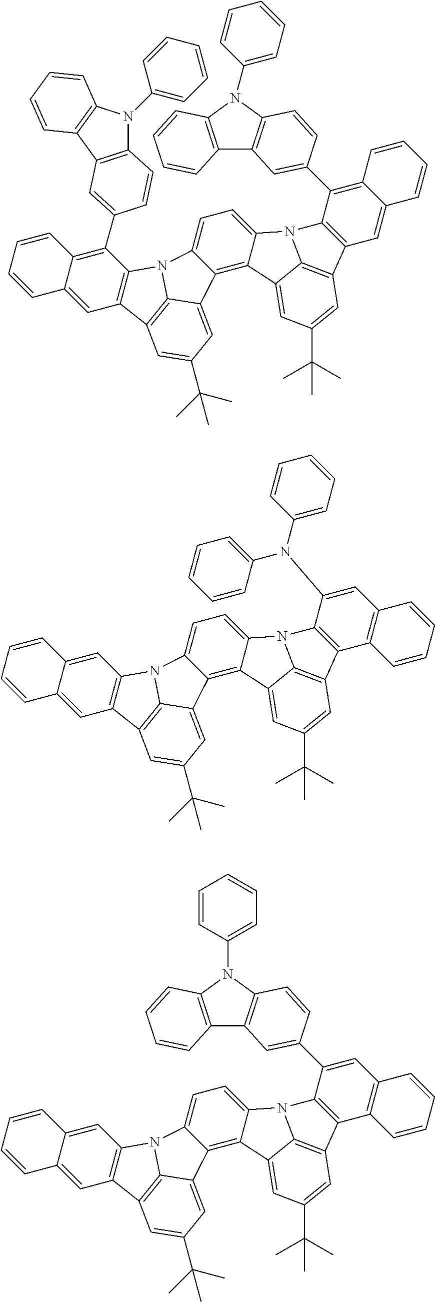 [SMILES: CC(C)(C)c1cc2c3cc4ccccc4c(-c4ccc5c(c4)c4ccccc4n5-c4ccccc4)c3n3c4ccc5c(c6cc(C(C)(C)C)cc7c8cc9ccccc9c(-c9ccc%10c(c9)c9ccccc9n%10-c9ccccc9)c8n5c76)c4c(c1)c23 ; CC(C)(C)c1cc2c3cc4ccccc4cc3n3c4ccc5c(c6cc(C(C)(C)C)cc7c8c9ccccc9cc(N(c9ccccc9)c9ccccc9)c8n5c67)c4c(c1)c23 ; CC(C)(C)c1cc2c3cc4ccccc4cc3n3c4ccc5c(c6cc(C(C)(C)C)cc7c8c9ccccc9cc(-c9ccc%10c(c9)c9ccccc9n%10-c9ccccc9)c8n5c67)c4c(c1)c23]

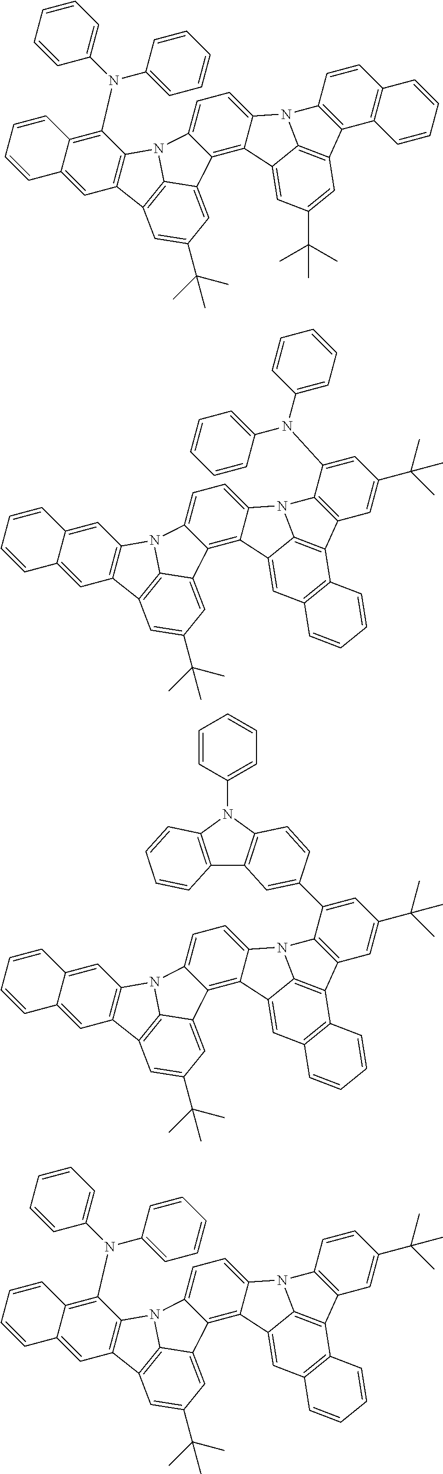

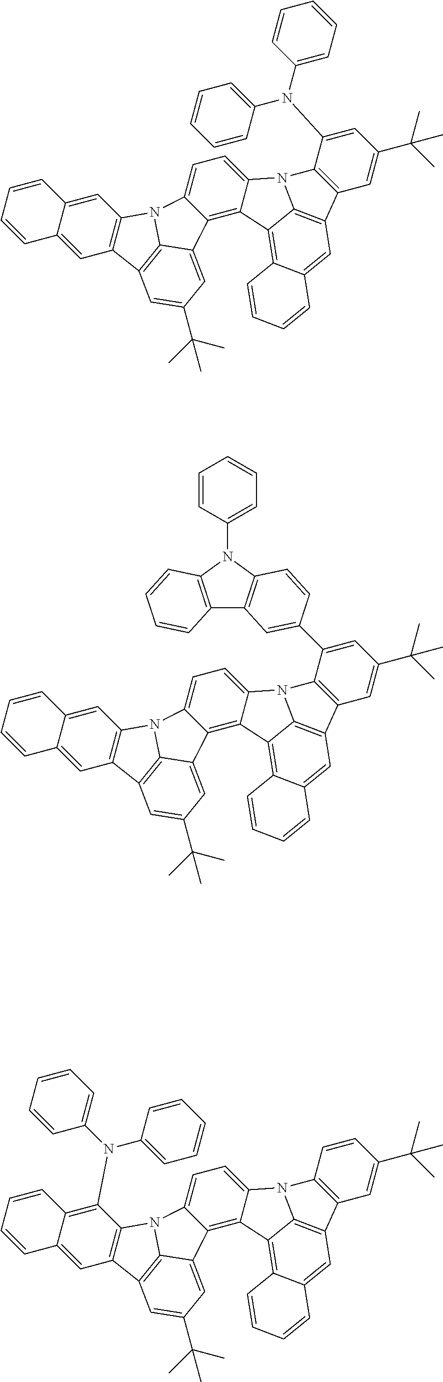 [SMILES: CC(C)(C)c1cc(N(c2ccccc2)c2ccccc2)c2c(c1)c1cc3ccccc3c3c4c5c6cc(C(C)(C)C)cc7c8cc9ccccc9cc8n(c5ccc4n2c13)c76 ; CC(C)(C)c1cc(-c2ccc3c(c2)c2ccccc2n3-c2ccccc2)c2c(c1)c1cc3ccccc3c3c4c5c6cc(C(C)(C)C)cc7c8cc9ccccc9cc8n(c5ccc4n2c13)c76 ; CC(C)(C)c1ccc2c(c1)c1cc3ccccc3c3c4c5c6cc(C(C)(C)C)cc7c8cc9ccccc9c(N(c9ccccc9)c9ccccc9)c8n(c5ccc4n2c13)c76]

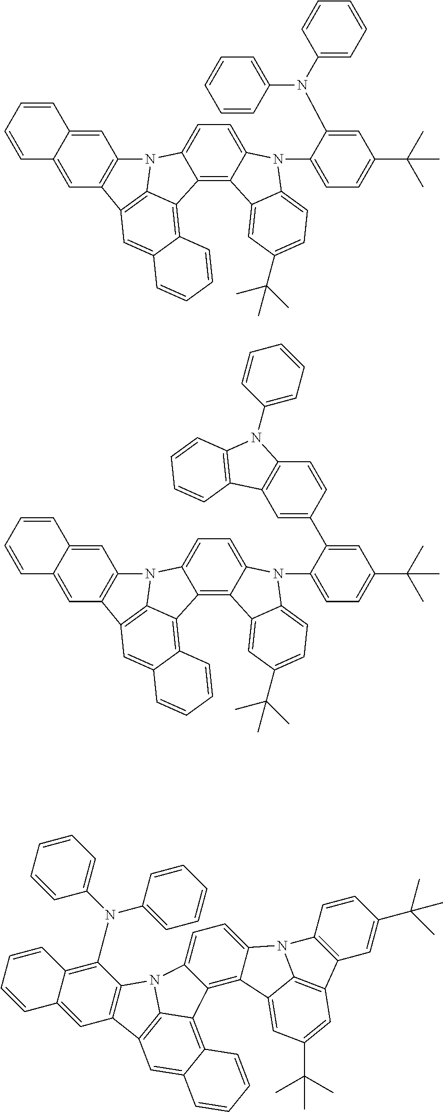

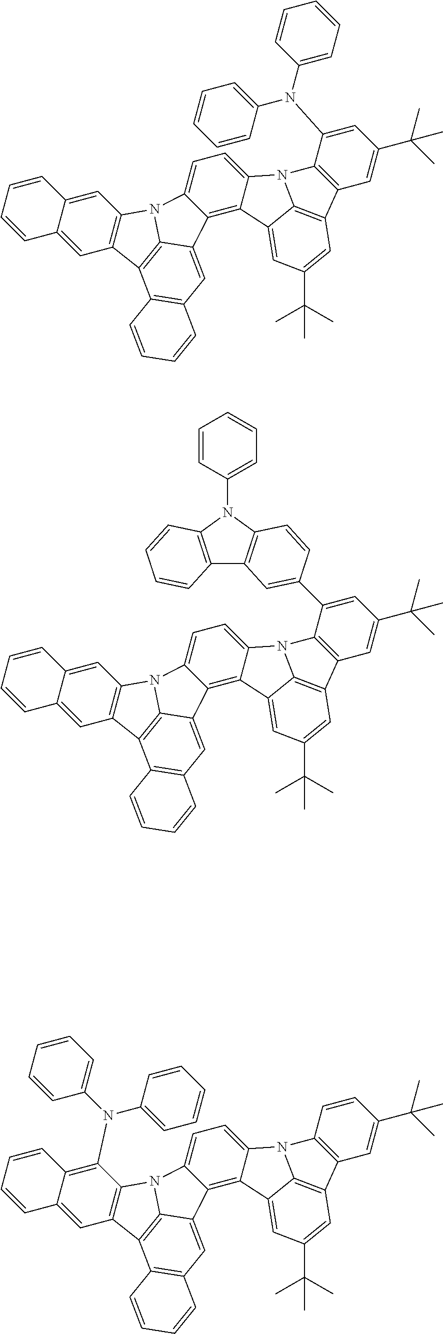 [SMILES: CC(C)(C)c1cc(N(c2ccccc2)c2ccccc2)c2c(c1)c1cc(C(C)(C)C)cc3c4c5c6cc7ccccc7c7c8cc9ccccc9cc8n(c5ccc4n2c13)c67 ; CC(C)(C)c1cc(-c2ccc3c(c2)c2ccccc2n3-c2ccccc2)c2c(c1)c1cc(C(C)(C)C)cc3c4c5c6cc7ccccc7c7c8cc9ccccc9cc8n(c5ccc4n2c13)c67 ; CC(C)(C)c1ccc2c(c1)c1cc(C(C)(C)C)cc3c4c5c6cc7ccccc7c7c8cc9ccccc9c(N(c9ccccc9)c9ccccc9)c8n(c5ccc4n2c13)c67]

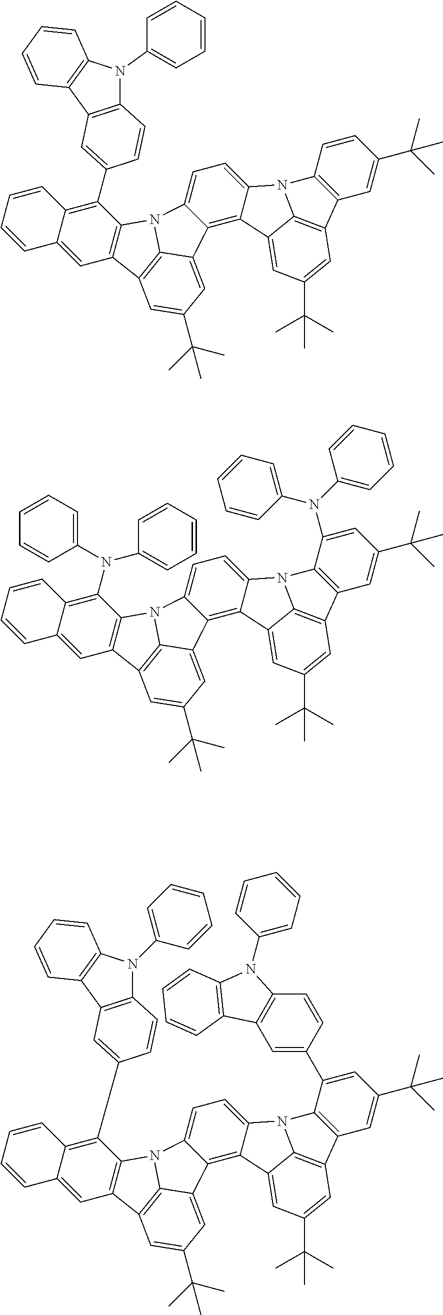 [SMILES: CC(C)(C)c1ccc2c(c1)c1cc(C(C)(C)C)cc3c4c5c6cc(C(C)(C)C)cc7c8cc9ccccc9c(-c9ccc%10c(c9)c9ccccc9n%10-c9ccccc9)c8n(c5ccc4n2c13)c76 ; CC(C)(C)c1cc(N(c2ccccc2)c2ccccc2)c2c(c1)c1cc(C(C)(C)C)cc3c4c5c6cc(C(C)(C)C)cc7c8cc9ccccc9c(N(c9ccccc9)c9ccccc9)c8n(c5ccc4n2c13)c76 ; CC(C)(C)c1cc(-c2ccc3c(c2)c2ccccc2n3-c2ccccc2)c2c(c1)c1cc(C(C)(C)C)cc3c4c5c6cc(C(C)(C)C)cc7c8cc9ccccc9c(-c9ccc%10c(c9)c9ccccc9n%10-c9ccccc9)c8n(c5ccc4n2c13)c76]

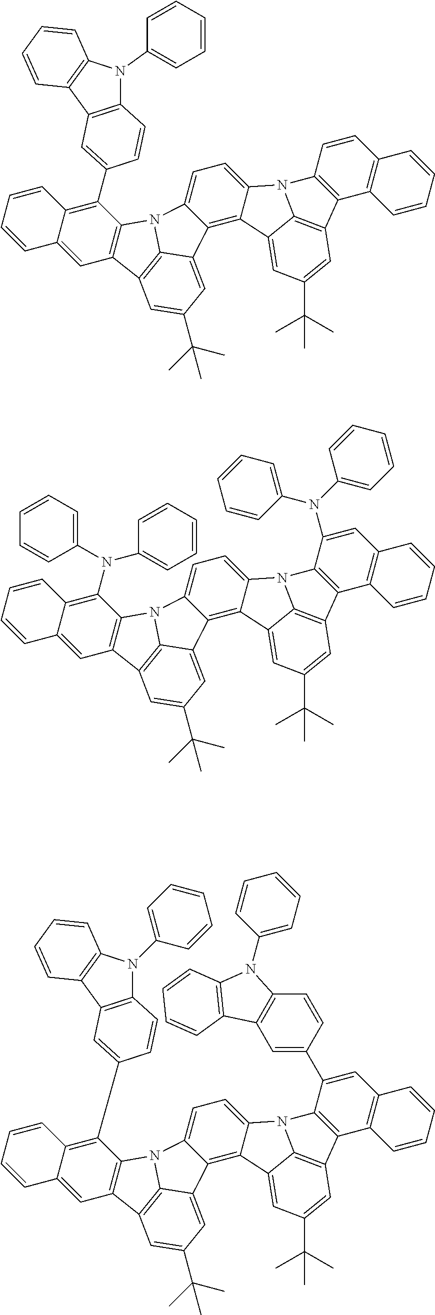 [SMILES: CC(C)(C)c1cc2c3c4ccccc4ccc3n3c4ccc5c(c6cc(C(C)(C)C)cc7c8cc9ccccc9c(-c9ccc%10c(c9)c9ccccc9n%10-c9ccccc9)c8n5c76)c4c(c1)c23 ; CC(C)(C)c1cc2c3cc4ccccc4c(N(c4ccccc4)c4ccccc4)c3n3c4ccc5c(c6cc(C(C)(C)C)cc7c8c9ccccc9cc(N(c9ccccc9)c9ccccc9)c8n5c67)c4c(c1)c23 ; CC(C)(C)c1cc2c3cc4ccccc4c(-c4ccc5c(c4)c4ccccc4n5-c4ccccc4)c3n3c4ccc5c(c6cc(C(C)(C)C)cc7c8c9ccccc9cc(-c9ccc%10c(c9)c9ccccc9n%10-c9ccccc9)c8n5c67)c4c(c1)c23]

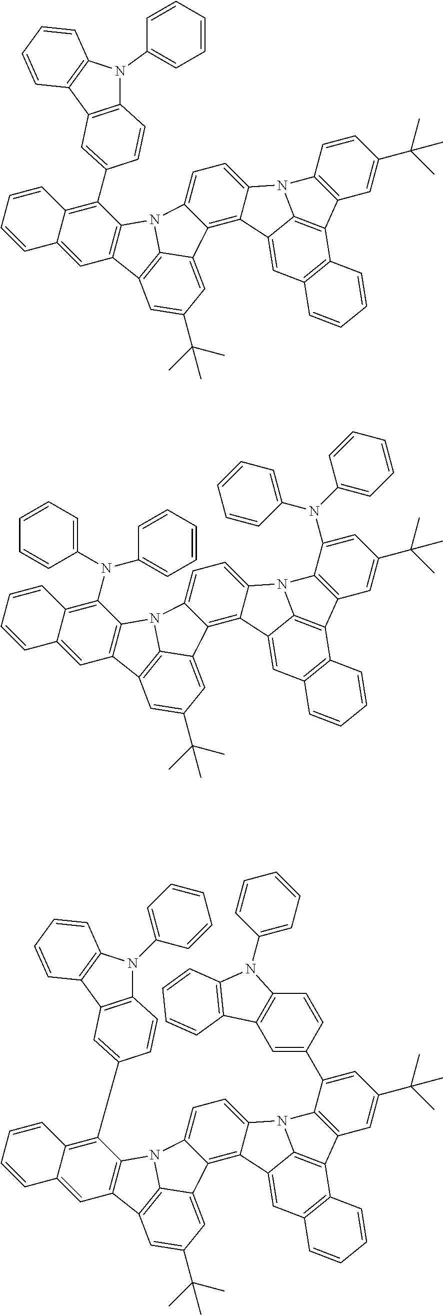 [SMILES: CC(C)(C)c1ccc2c(c1)c1c3ccccc3cc3c4c5c6cc(C(C)(C)C)cc7c8cc9ccccc9c(-c9ccc%10c(c9)c9ccccc9n%10-c9ccccc9)c8n(c5ccc4n2c31)c76 ; CC(C)(C)c1cc(N(c2ccccc2)c2ccccc2)c2c(c1)c1c3ccccc3cc3c4c5c6cc(C(C)(C)C)cc7c8cc9ccccc9c(N(c9ccccc9)c9ccccc9)c8n(c5ccc4n2c31)c76 ; CC(C)(C)c1cc(-c2ccc3c(c2)c2ccccc2n3-c2ccccc2)c2c(c1)c1c3ccccc3cc3c4c5c6cc(C(C)(C)C)cc7c8cc9ccccc9c(-c9ccc%10c(c9)c9ccccc9n%10-c9ccccc9)c8n(c5ccc4n2c31)c76]

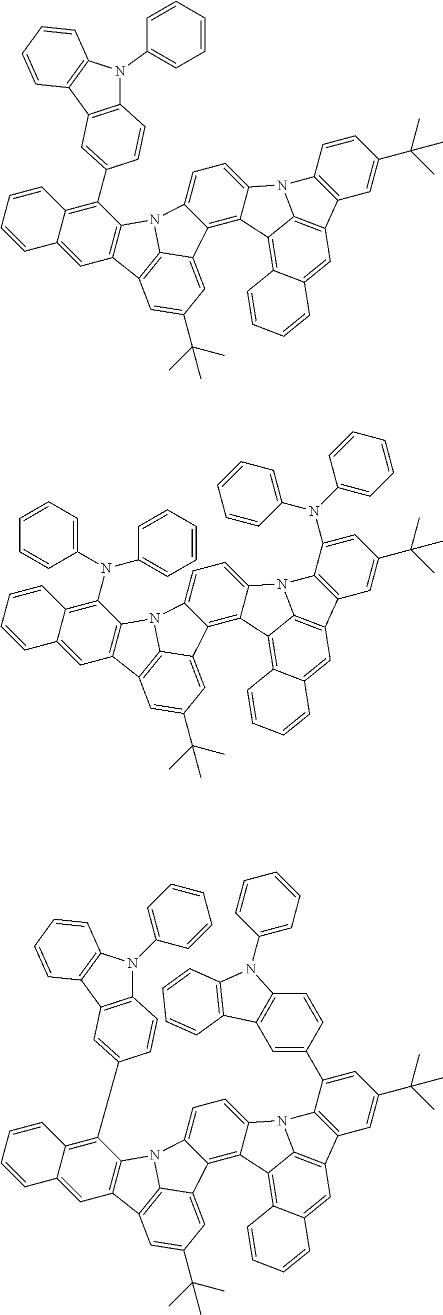 [SMILES: CC(C)(C)c1ccc2c(c1)c1cc3ccccc3c3c4c5c6cc(C(C)(C)C)cc7c8cc9ccccc9c(-c9ccc%10c(c9)c9ccccc9n%10-c9ccccc9)c8n(c5ccc4n2c13)c76 ; CC(C)(C)c1cc(N(c2ccccc2)c2ccccc2)c2c(c1)c1cc3ccccc3c3c4c5c6cc(C(C)(C)C)cc7c8cc9ccccc9c(N(c9ccccc9)c9ccccc9)c8n(c5ccc4n2c13)c76 ; CC(C)(C)c1cc(-c2ccc3c(c2)c2ccccc2n3-c2ccccc2)c2c(c1)c1cc3ccccc3c3c4c5c6cc(C(C)(C)C)cc7c8cc9ccccc9c(-c9ccc%10c(c9)c9ccccc9n%10-c9ccccc9)c8n(c5ccc4n2c13)c76]

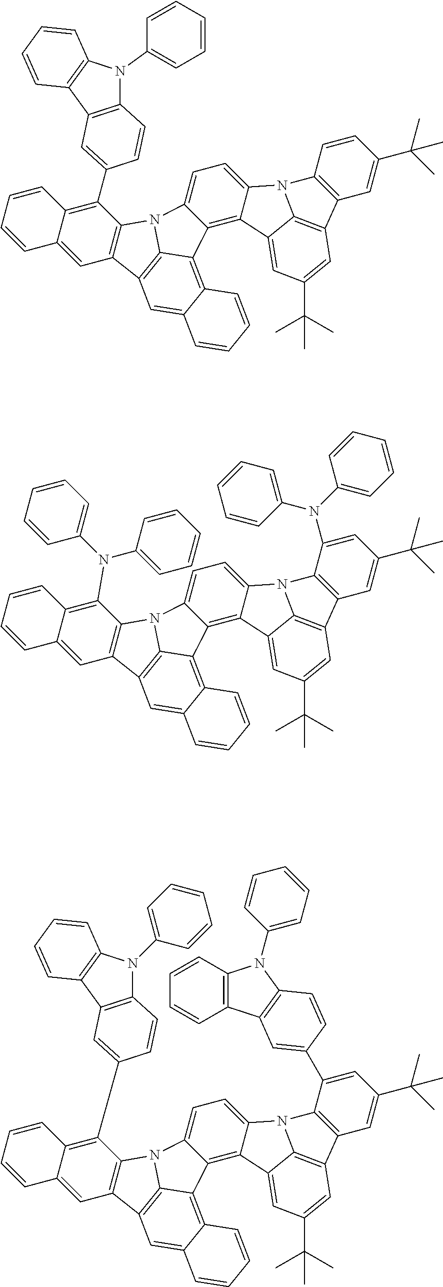 [SMILES: CC(C)(C)c1ccc2c(c1)c1cc(C(C)(C)C)cc3c4c5c6c7ccccc7cc7c8cc9ccccc9c(-c9ccc%10c(c9)c9ccccc9n%10-c9ccccc9)c8n(c5ccc4n2c13)c76 ; CC(C)(C)c1cc(N(c2ccccc2)c2ccccc2)c2c(c1)c1cc(C(C)(C)C)cc3c4c5c6c7ccccc7cc7c8cc9ccccc9c(N(c9ccccc9)c9ccccc9)c8n(c5ccc4n2c13)c76 ; CC(C)(C)c1cc(-c2ccc3c(c2)c2ccccc2n3-c2ccccc2)c2c(c1)c1cc(C(C)(C)C)cc3c4c5c6c7ccccc7cc7c8cc9ccccc9c(-c9ccc%10c(c9)c9ccccc9n%10-c9ccccc9)c8n(c5ccc4n2c13)c76]

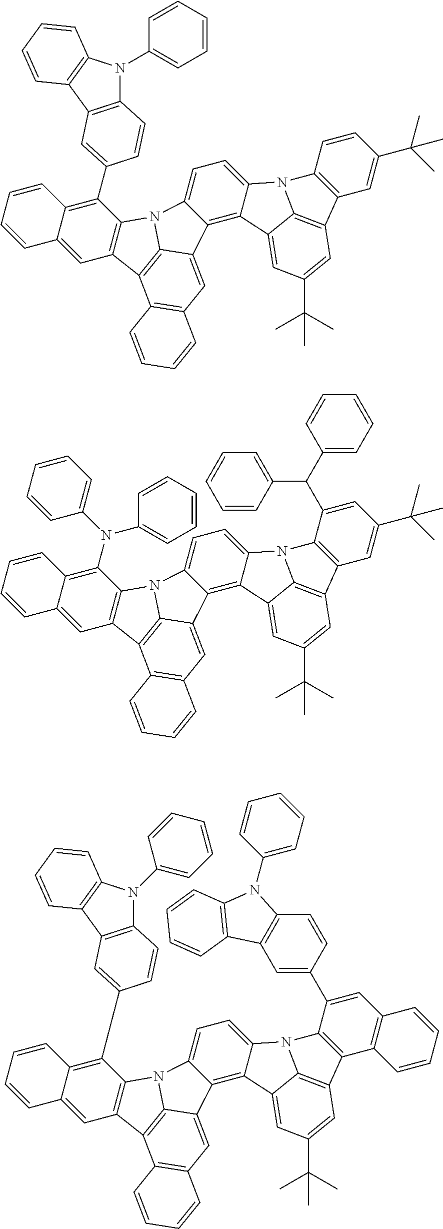 [SMILES: CC(C)(C)c1ccc2c(c1)c1cc(C(C)(C)C)cc3c4c5c6cc7ccccc7c7c8cc9ccccc9c(-c9ccc%10c(c9)c9ccccc9n%10-c9ccccc9)c8n(c5ccc4n2c13)c67 ; CC(C)(C)c1cc(C(c2ccccc2)c2ccccc2)c2c(c1)c1cc(C(C)(C)C)cc3c4c5c6cc7ccccc7c7c8cc9ccccc9c(N(c9ccccc9)c9ccccc9)c8n(c5ccc4n2c13)c67 ; CC(C)(C)c1cc2c3c4c5cc6ccccc6c6c7cc8ccccc8c(-c8ccc9c(c8)c8ccccc8n9-c8ccccc8)c7n(c4ccc3n3c4c(-c7ccc8c(c7)c7ccccc7n8-c7ccccc7)cc7ccccc7c4c(c1)c23)c56]

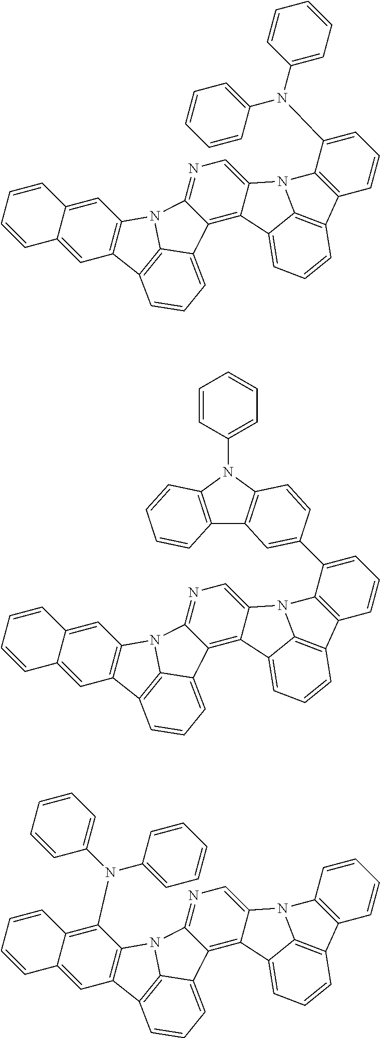 [SMILES: c1ccc(N(c2ccccc2)c2cccc3c4cccc5c6c7c8cccc9c%10cc%11ccccc%11cc%10n(c7ncc6n(c23)c45)c98)cc1 ; c1ccc(-n2c3ccccc3c3cc(-c4cccc5c6cccc7c8c9c%10cccc%11c%12cc%13ccccc%13cc%12n(c9ncc8n(c45)c67)c%11%10)ccc32)cc1 ; c1ccc(N(c2ccccc2)c2c3ccccc3cc3c4cccc5c6c7c8cccc9c%10ccccc%10n(c7cnc6n(c23)c45)c98)cc1]

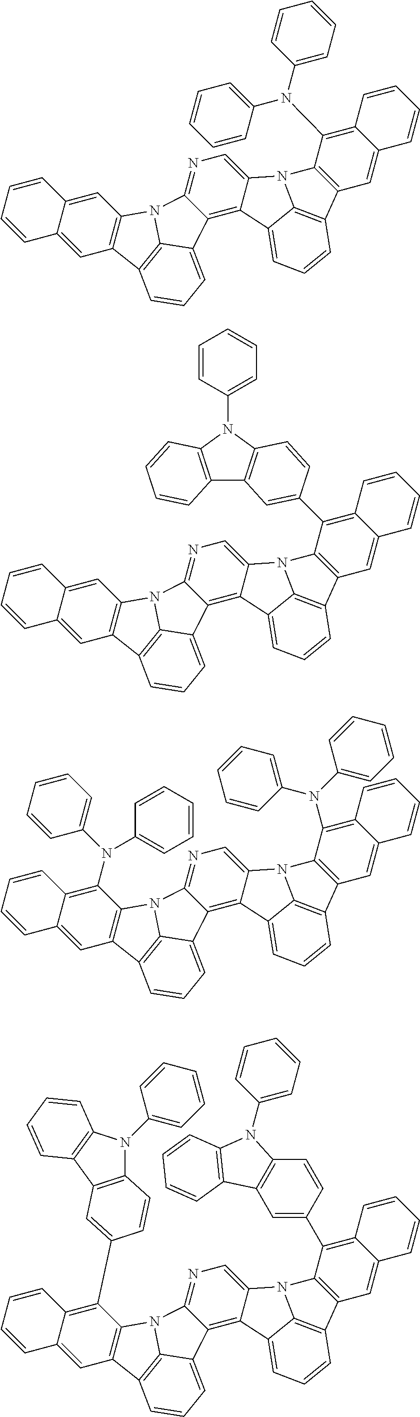

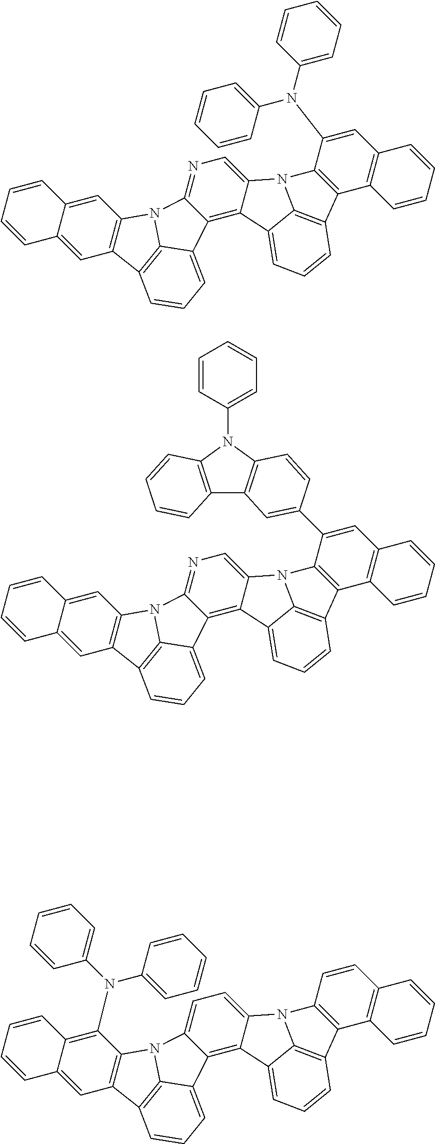 [SMILES: c1ccc(N(c2ccccc2)c2cc3ccccc3c3c4cccc5c6c7c8cccc9c%10cc%11ccccc%11cc%10n(c7ncc6n(c23)c54)c98)cc1 ; c1ccc(-n2c3ccccc3c3cc(-c4cc5ccccc5c5c6cccc7c8c9c%10cccc%11c%12cc%13ccccc%13cc%12n(c9ncc8n(c45)c76)c%11%10)ccc32)cc1 ; c1ccc(N(c2ccccc2)c2c3ccccc3cc3c4cccc5c6c7c8cccc9c%10c%11ccccc%11ccc%10n(c7ccc6n(c23)c45)c98)cc1]

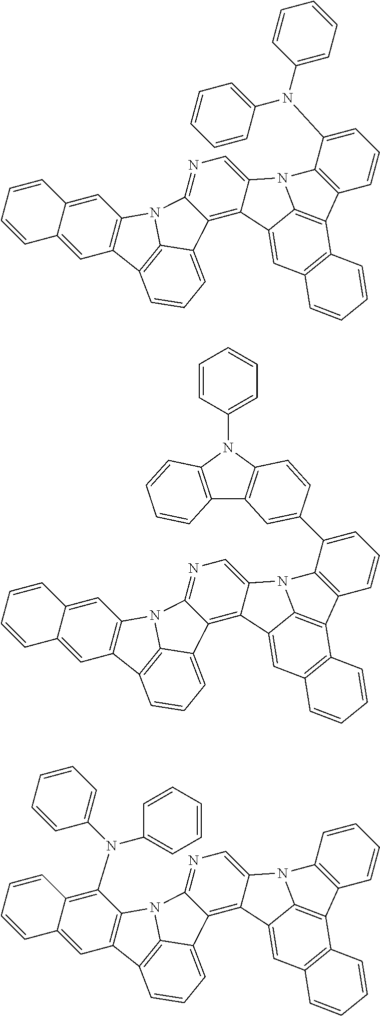 [SMILES: c1ccc(N(c2ccccc2)c2cccc3c4c5ccccc5cc5c6c7c8cccc9c%10cc%11ccccc%11cc%10n(c7ncc6n(c23)c54)c98)cc1 ; c1ccc(-n2c3ccccc3c3cc(-c4cccc5c6c7ccccc7cc7c8c9c%10cccc%11c%12cc%13ccccc%13cc%12n(c9ncc8n(c45)c76)c%11%10)ccc32)cc1 ; c1ccc(N(c2ccccc2)c2c3ccccc3cc3c4cccc5c6c7c8cc9ccccc9c9c%10ccccc%10n(c7cnc6n(c23)c45)c89)cc1]

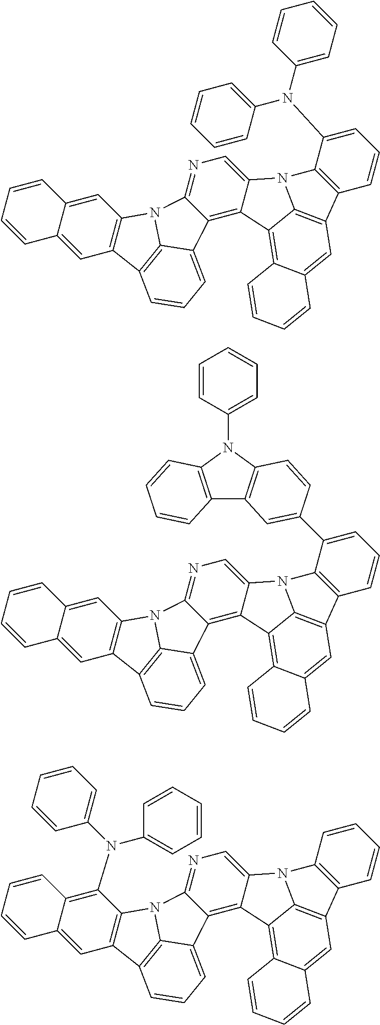

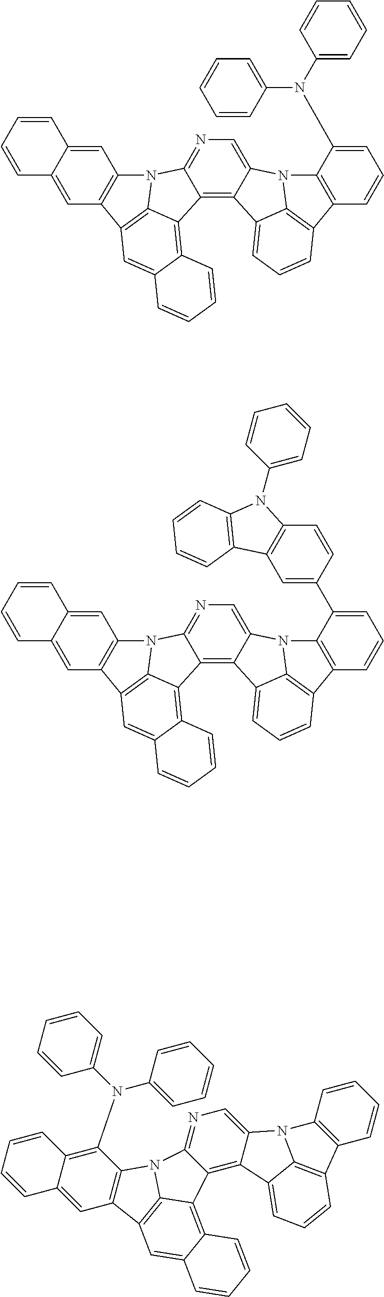 [SMILES: c1ccc(N(c2ccccc2)c2cccc3c4cccc5c6c7c8c9ccccc9cc9c%10cc%11ccccc%11cc%10n(c7ncc6n(c23)c45)c98)cc1 ; c1ccc(-n2c3ccccc3c3cc(-c4cccc5c6cccc7c8c9c%10c%11ccccc%11cc%11c%12cc%13ccccc%13cc%12n(c9ncc8n(c45)c67)c%11%10)ccc32)cc1 ; c1ccc(N(c2ccccc2)c2c3ccccc3cc3c4cc5ccccc5c5c6c7c8cccc9c%10ccccc%10n(c7cnc6n(c23)c45)c98)cc1]

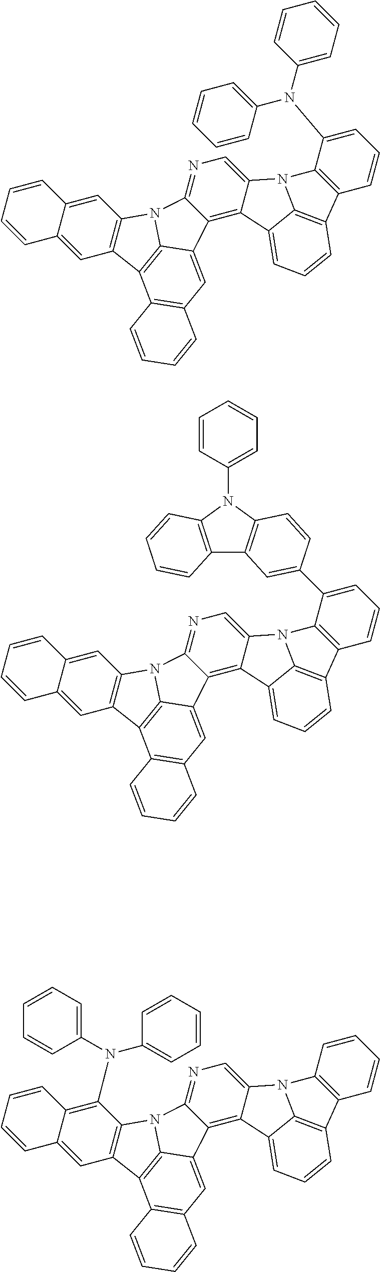 [SMILES: c1ccc(N(c2ccccc2)c2cccc3c4cccc5c6c7c8cc9ccccc9c9c%10cc%11ccccc%11cc%10n(c7ncc6n(c23)c45)c89)cc1 ; c1ccc(-n2c3ccccc3c3cc(-c4cccc5c6cccc7c8c9c%10cc%11ccccc%11c%11c%12cc%13ccccc%13cc%12n(c9ncc8n(c45)c67)c%10%11)ccc32)cc1 ; c1ccc(N(c2ccccc2)c2c3ccccc3cc3c4c5ccccc5cc5c6c7c8cccc9c%10ccccc%10n(c7cnc6n(c23)c54)c98)cc1]

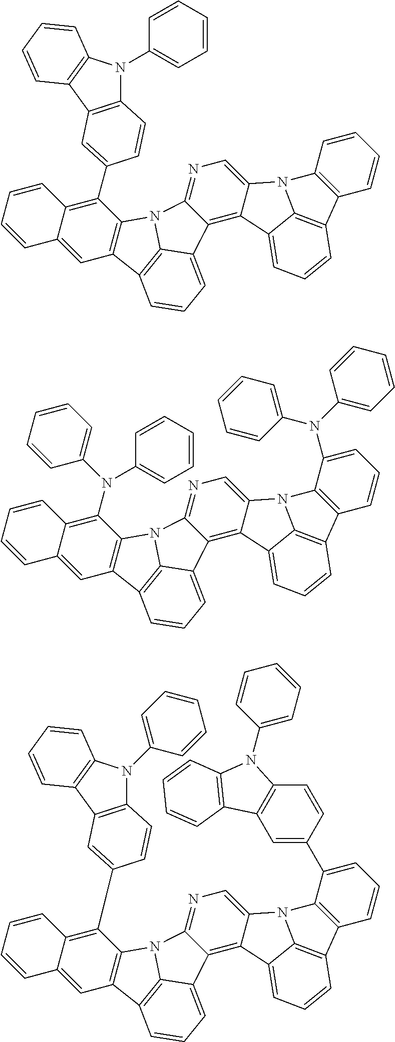 [SMILES: c1ccc(-n2c3ccccc3c3cc(-c4c5ccccc5cc5c6cccc7c8c9c%10cccc%11c%12ccccc%12n(c9cnc8n(c45)c67)c%11%10)ccc32)cc1 ; c1ccc(N(c2ccccc2)c2cccc3c4cccc5c6c7c8cccc9c%10cc%11ccccc%11c(N(c%11ccccc%11)c%11ccccc%11)c%10n(c7ncc6n(c23)c45)c98)cc1 ; c1ccc(-n2c3ccccc3c3cc(-c4cccc5c6cccc7c8c9c%10cccc%11c%12cc%13ccccc%13c(-c%13ccc%14c(c%13)c%13ccccc%13n%14-c%13ccccc%13)c%12n(c9ncc8n(c45)c67)c%11%10)ccc32)cc1]

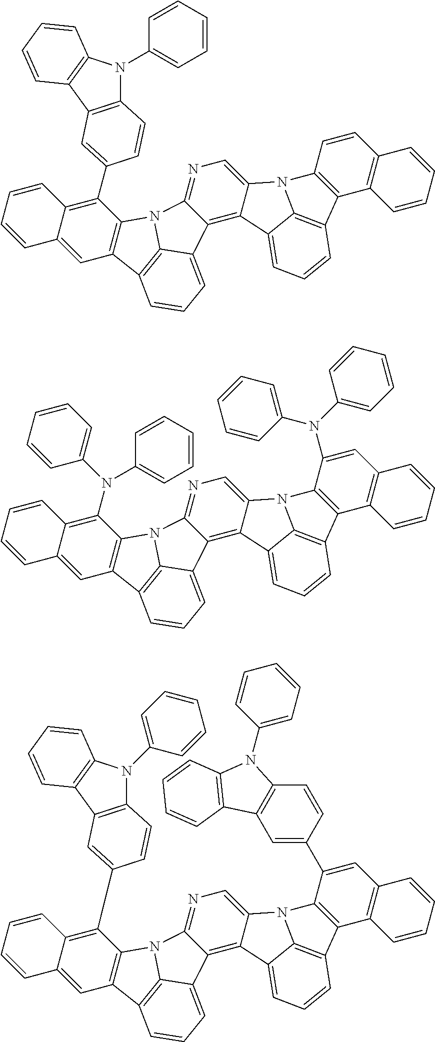 [SMILES: c1ccc(-n2c3ccccc3c3cc(-c4c5ccccc5cc5c6cccc7c8c9c%10cccc%11c%12c%13ccccc%13ccc%12n(c9cnc8n(c45)c67)c%11%10)ccc32)cc1 ; c1ccc(N(c2ccccc2)c2cc3ccccc3c3c4cccc5c6c7c8cccc9c%10cc%11ccccc%11c(N(c%11ccccc%11)c%11ccccc%11)c%10n(c7ncc6n(c23)c54)c98)cc1 ; c1ccc(-n2c3ccccc3c3cc(-c4cc5ccccc5c5c6cccc7c8c9c%10cccc%11c%12cc%13ccccc%13c(-c%13ccc%14c(c%13)c%13ccccc%13n%14-c%13ccccc%13)c%12n(c9ncc8n(c45)c76)c%11%10)ccc32)cc1]

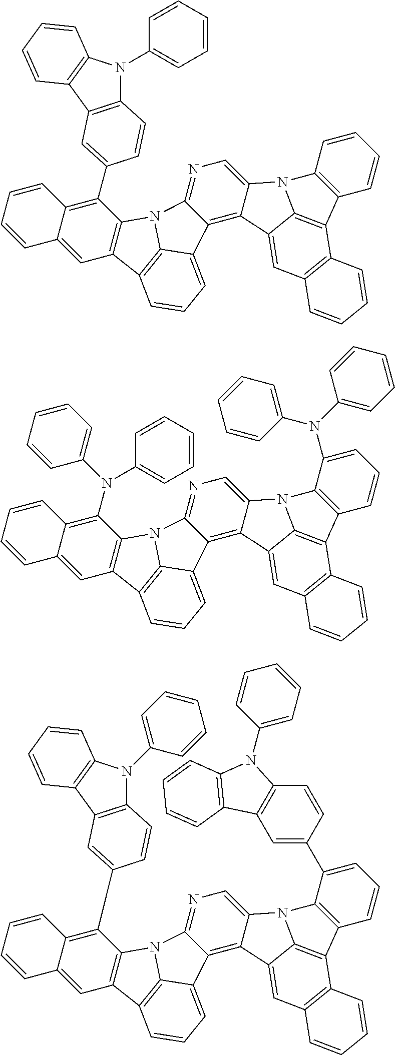

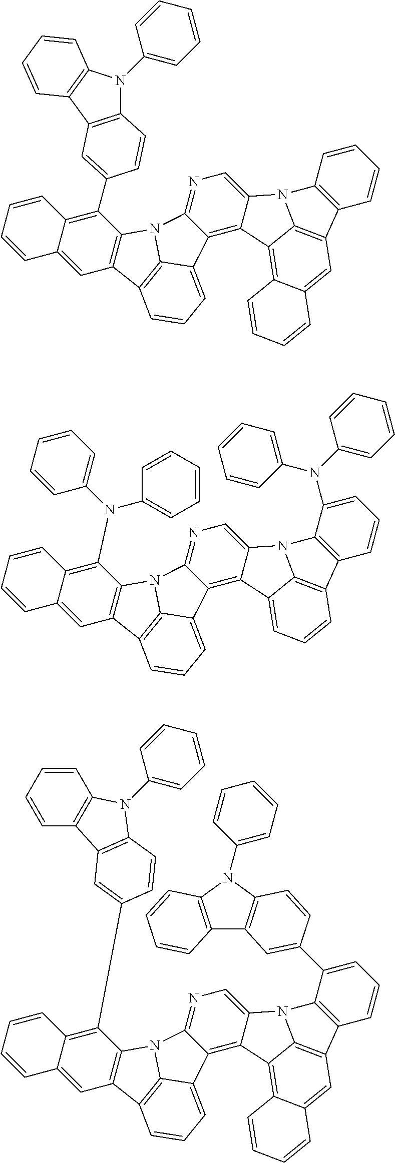 [SMILES: c1ccc(-n2c3ccccc3c3cc(-c4c5ccccc5cc5c6cccc7c8c9c%10c%11ccccc%11cc%11c%12ccccc%12n(c9cnc8n(c45)c67)c%11%10)ccc32)cc1 ; c1ccc(N(c2ccccc2)c2cccc3c4cccc5c6c7c8cccc9c%10cc%11ccccc%11c(N(c%11ccccc%11)c%11ccccc%11)c%10n(c7ncc6n(c23)c45)c98)cc1 ; c1ccc(-n2c3ccccc3c3cc(-c4cccc5c6cc7ccccc7c7c8c9c%10cccc%11c%12cc%13ccccc%13c(-c%13ccc%14c(c%13)c%13ccccc%13n%14-c%13ccccc%13)c%12n(c9ncc8n(c45)c67)c%11%10)ccc32)cc1]

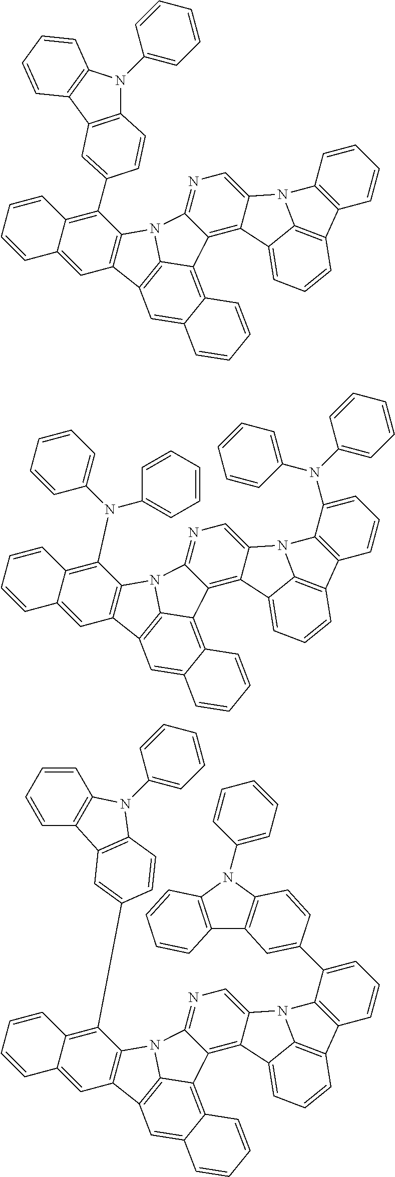

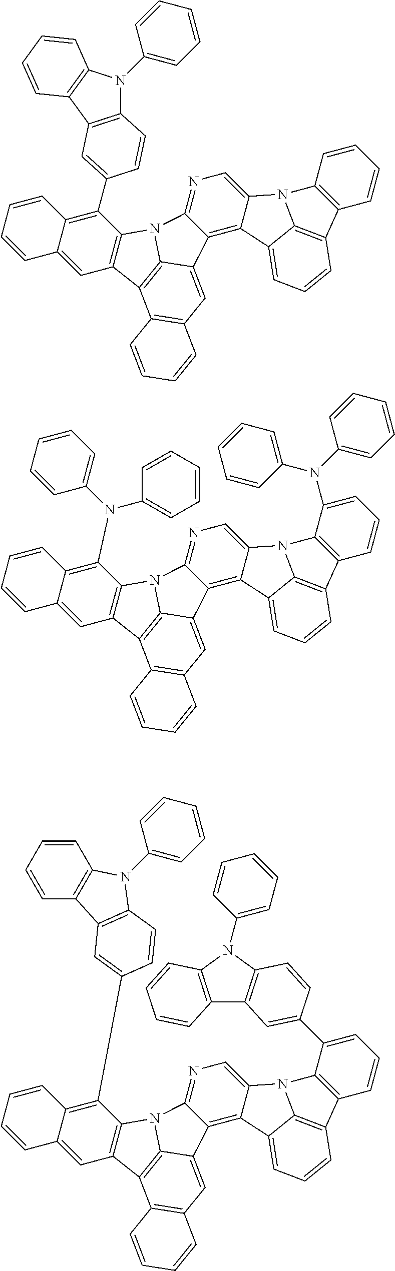 [SMILES: c1ccc(-n2c3ccccc3c3cc(-c4c5ccccc5cc5c6c7ccccc7cc7c8c9c%10cccc%11c%12ccccc%12n(c9cnc8n(c45)c76)c%11%10)ccc32)cc1 ; c1ccc(N(c2ccccc2)c2cccc3c4cccc5c6c7c8cc9ccccc9c9c%10cc%11ccccc%11c(N(c%11ccccc%11)c%11ccccc%11)c%10n(c7ncc6n(c23)c45)c89)cc1 ; c1ccc(-n2c3ccccc3c3cc(-c4cccc5c6cccc7c8c9c%10cc%11ccccc%11c%11c%12cc%13ccccc%13c(-c%13ccc%14c(c%13)c%13ccccc%13n%14-c%13ccccc%13)c%12n(c9ncc8n(c45)c67)c%10%11)ccc32)cc1]

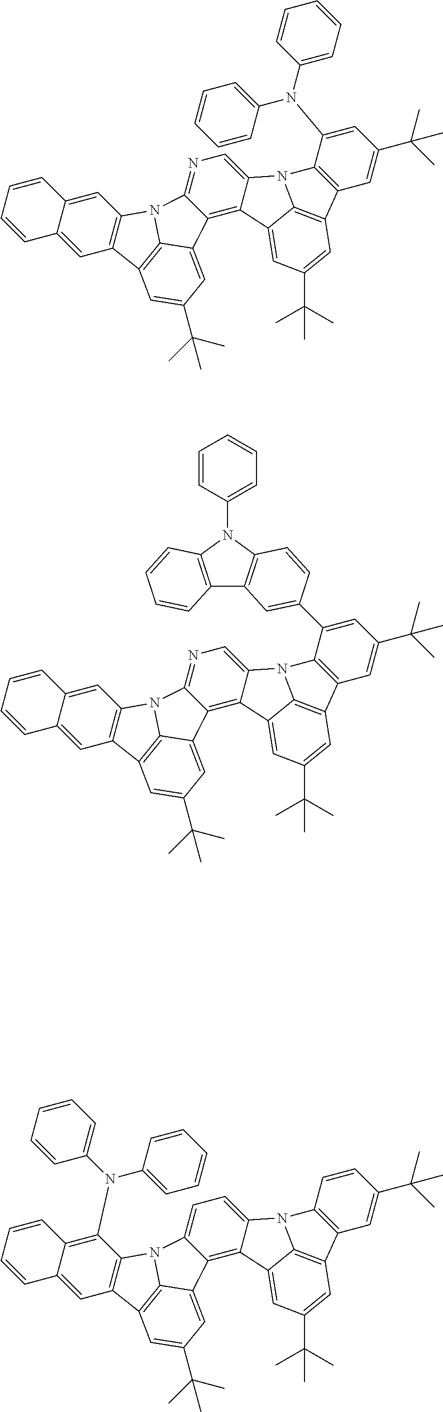 [SMILES: CC(C)(C)c1cc(N(c2ccccc2)c2ccccc2)c2c(c1)c1cc(C(C)(C)C)cc3c4c5c6cc(C(C)(C)C)cc7c8cc9ccccc9cc8n(c5ncc4n2c13)c76 ; CC(C)(C)c1cc(-c2ccc3c(c2)c2ccccc2n3-c2ccccc2)c2c(c1)c1cc(C(C)(C)C)cc3c4c5c6cc(C(C)(C)C)cc7c8cc9ccccc9cc8n(c5ncc4n2c13)c76 ; CC(C)(C)c1ccc2c(c1)c1cc(C(C)(C)C)cc3c4c5c6cc(C(C)(C)C)cc7c8cc9ccccc9c(N(c9ccccc9)c9ccccc9)c8n(c5ccc4n2c13)c76]

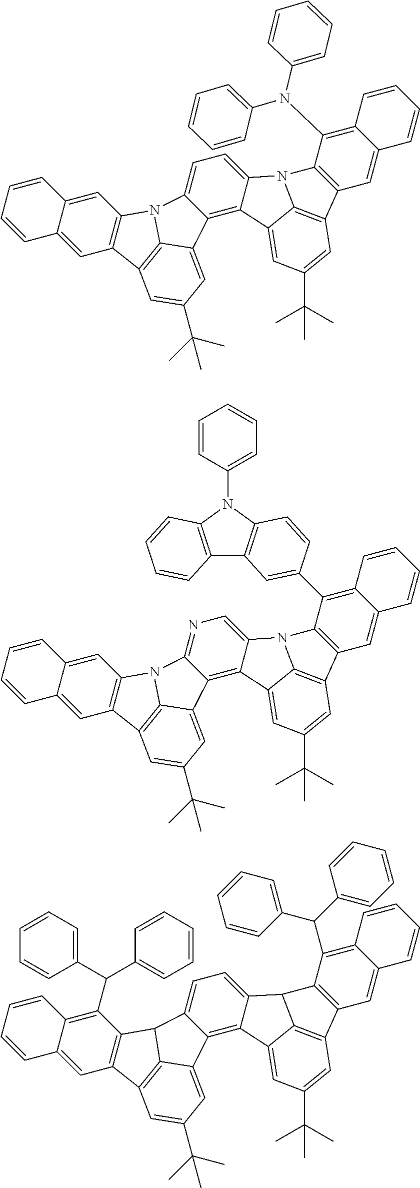

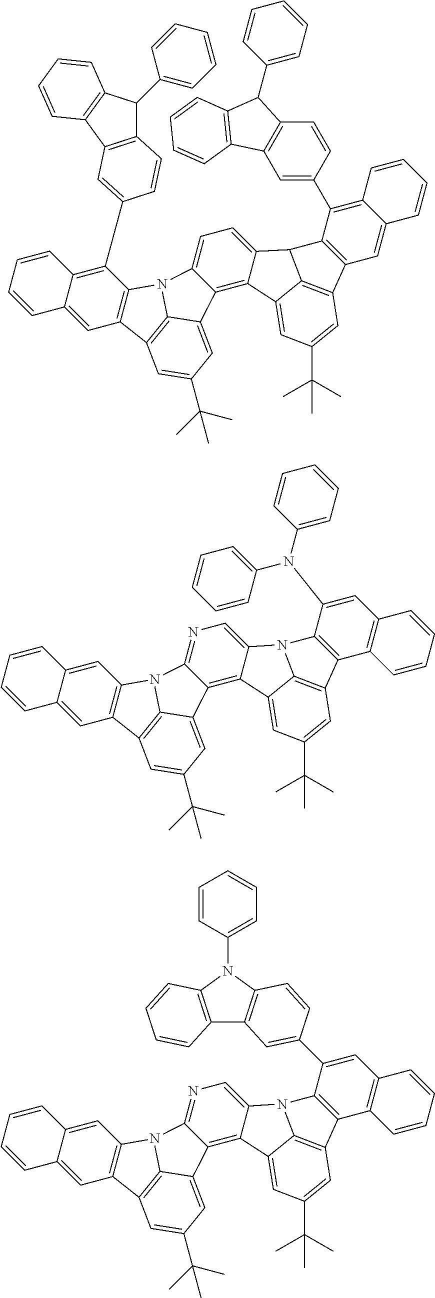

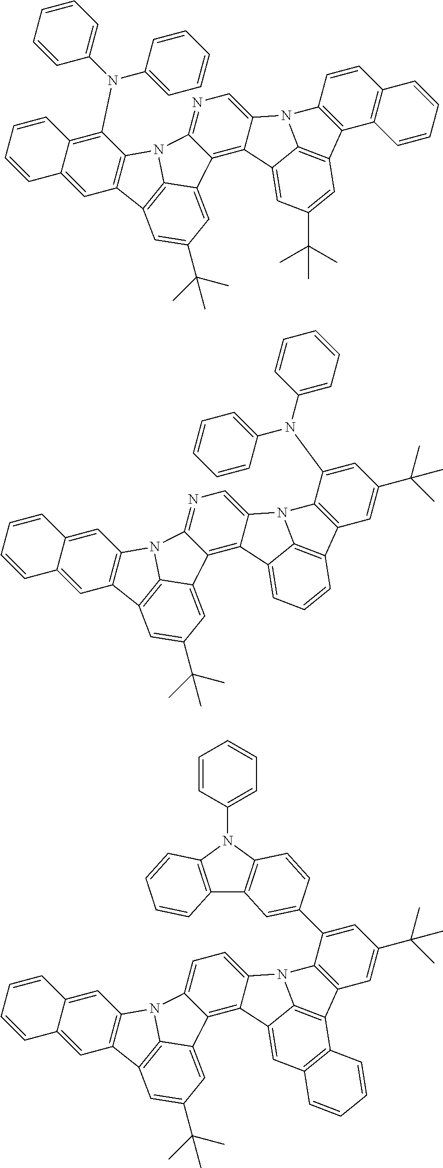

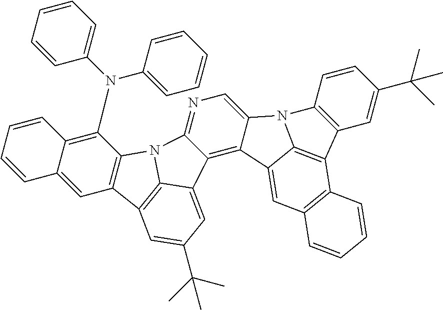 [SMILES: CC(C)(C)c1ccc2c(c1)c1c3ccccc3cc3c4c5c6cc(C(C)(C)C)cc7c8cc9ccccc9c(N(c9ccccc9)c9ccccc9)c8n(c5ncc4n2c31)c76]

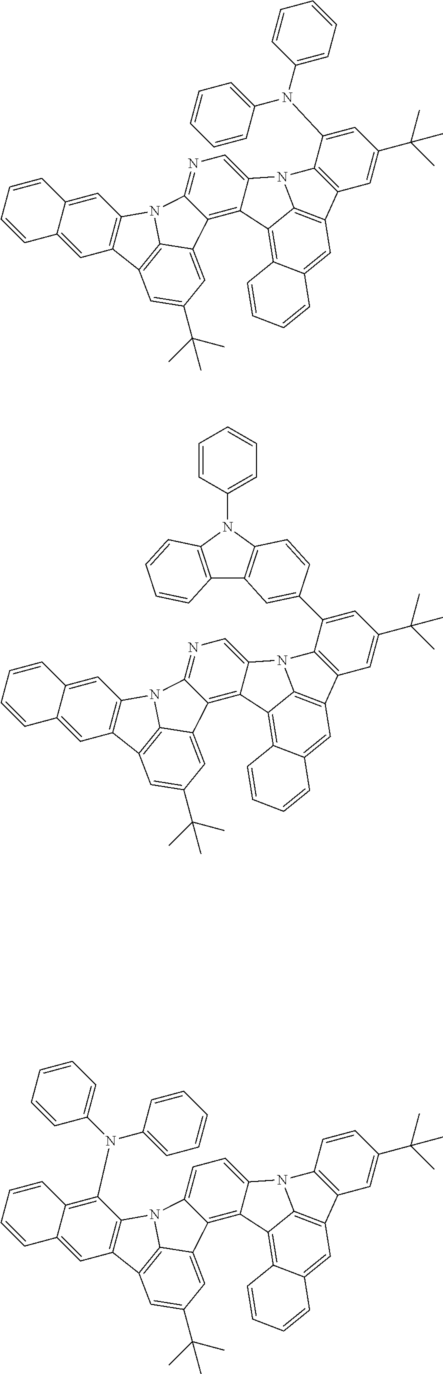 [SMILES: CC(C)(C)c1cc(N(c2ccccc2)c2ccccc2)c2c(c1)c1cc3ccccc3c3c4c5c6cc(C(C)(C)C)cc7c8cc9ccccc9cc8n(c5ncc4n2c13)c76 ; CC(C)(C)c1cc(-c2ccc3c(c2)c2ccccc2n3-c2ccccc2)c2c(c1)c1cc3ccccc3c3c4c5c6cc(C(C)(C)C)cc7c8cc9ccccc9cc8n(c5ncc4n2c13)c76 ; CC(C)(C)c1ccc2c(c1)c1cc3ccccc3c3c4c5c6cc(C(C)(C)C)cc7c8cc9ccccc9c(N(c9ccccc9)c9ccccc9)c8n(c5ccc4n2c13)c76]

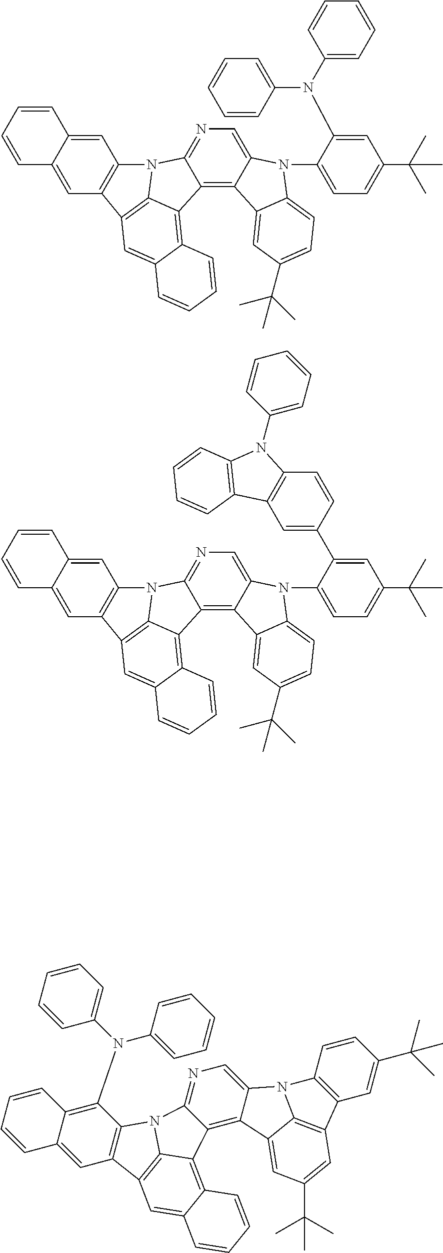

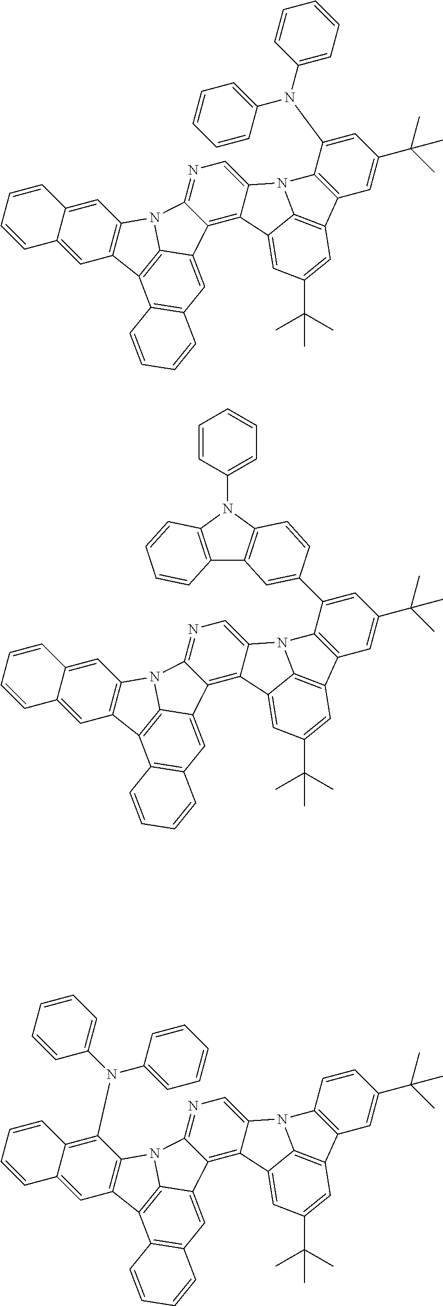 [SMILES: CC(C)(C)c1cc(N(c2ccccc2)c2ccccc2)c2c(c1)c1cc(C(C)(C)C)cc3c4c5c6cc7ccccc7c7c8cc9ccccc9cc8n(c5ncc4n2c13)c67 ; CC(C)(C)c1cc(-c2ccc3c(c2)c2ccccc2n3-c2ccccc2)c2c(c1)c1cc(C(C)(C)C)cc3c4c5c6cc7ccccc7c7c8cc9ccccc9cc8n(c5ncc4n2c13)c67 ; CC(C)(C)c1ccc2c(c1)c1cc(C(C)(C)C)cc3c4c5c6cc7ccccc7c7c8cc9ccccc9c(N(c9ccccc9)c9ccccc9)c8n(c5ncc4n2c13)c67]

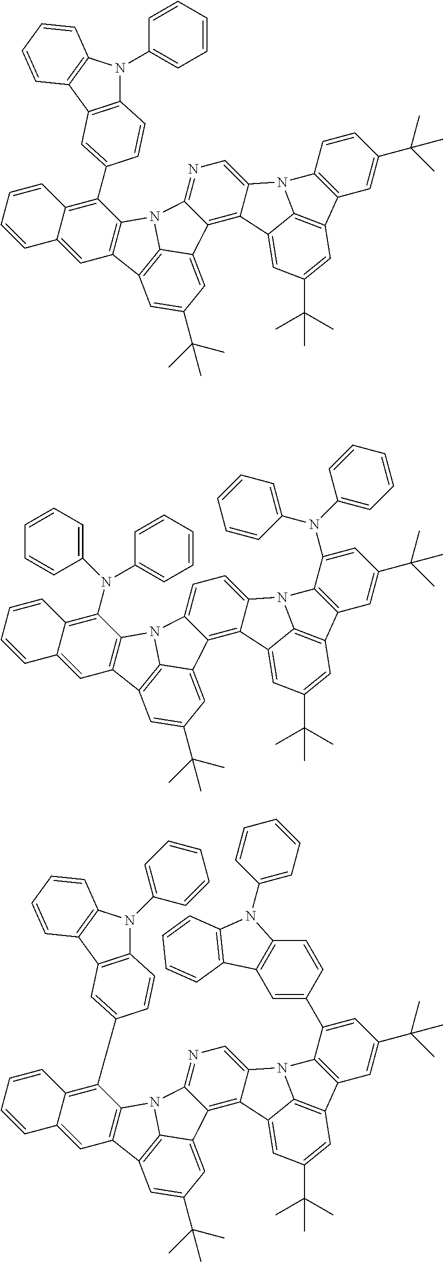 [SMILES: CC(C)(C)c1ccc2c(c1)c1cc(C(C)(C)C)cc3c4c5c6cc(C(C)(C)C)cc7c8cc9ccccc9c(-c9ccc%10c(c9)c9ccccc9n%10-c9ccccc9)c8n(c5ncc4n2c13)c76 ; CC(C)(C)c1cc(N(c2ccccc2)c2ccccc2)c2c(c1)c1cc(C(C)(C)C)cc3c4c5c6cc(C(C)(C)C)cc7c8cc9ccccc9c(N(c9ccccc9)c9ccccc9)c8n(c5ccc4n2c13)c76 ; CC(C)(C)c1cc(-c2ccc3c(c2)c2ccccc2n3-c2ccccc2)c2c(c1)c1cc(C(C)(C)C)cc3c4c5c6cc(C(C)(C)C)cc7c8cc9ccccc9c(-c9ccc%10c(c9)c9ccccc9n%10-c9ccccc9)c8n(c5ncc4n2c13)c76]

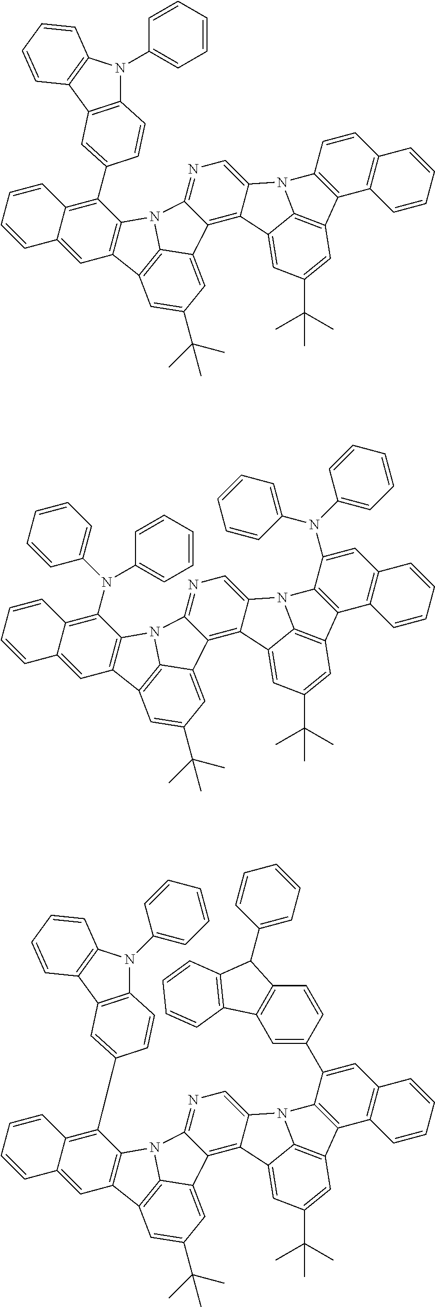 [SMILES: CC(C)(C)c1cc2c3c4ccccc4ccc3n3c4cnc5c(c6cc(C(C)(C)C)cc7c8cc9ccccc9c(-c9ccc%10c(c9)c9ccccc9n%10-c9ccccc9)c8n5c76)c4c(c1)c23 ; CC(C)(C)c1cc2c3c4c5cc(C(C)(C)C)cc6c7cc8ccccc8c(N(c8ccccc8)c8ccccc8)c7n(c4ncc3n3c4c(N(c7ccccc7)c7ccccc7)cc7ccccc7c4c(c1)c23)c65 ; CC(C)(C)c1cc2c3c4c5cc(C(C)(C)C)cc6c7cc8ccccc8c(-c8ccc9c(c8)c8ccccc8n9-c8ccccc8)c7n(c4ncc3n3c4c(-c7ccc8c(c7)-c7ccccc7C8c7ccccc7)cc7ccccc7c4c(c1)c23)c65]

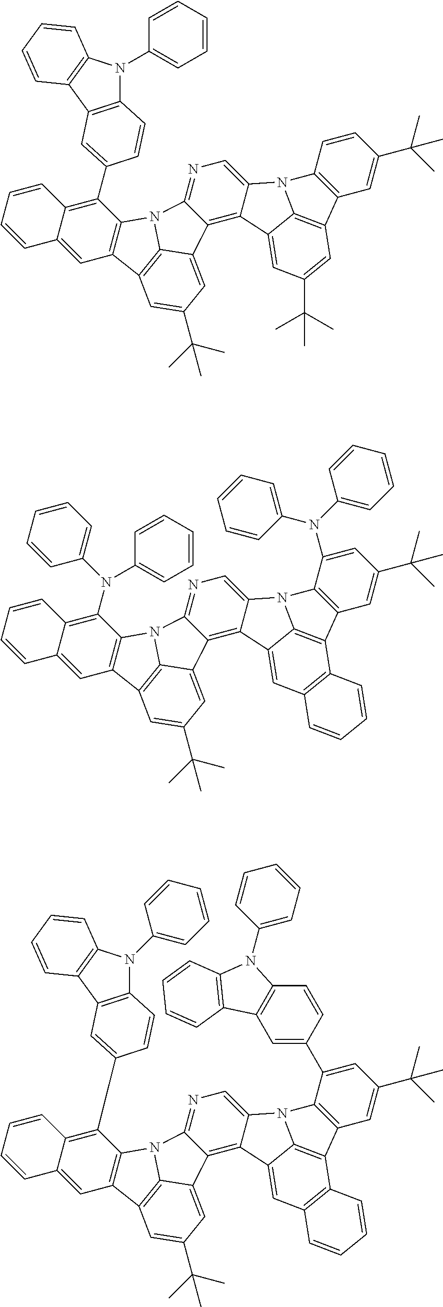 [SMILES: CC(C)(C)c1ccc2c(c1)c1cc(C(C)(C)C)cc3c4c5c6cc(C(C)(C)C)cc7c8cc9ccccc9c(-c9ccc%10c(c9)c9ccccc9n%10-c9ccccc9)c8n(c5ncc4n2c13)c76 ; CC(C)(C)c1cc(N(c2ccccc2)c2ccccc2)c2c(c1)c1c3ccccc3cc3c4c5c6cc(C(C)(C)C)cc7c8cc9ccccc9c(N(c9ccccc9)c9ccccc9)c8n(c5ncc4n2c31)c76 ; CC(C)(C)c1cc(-c2ccc3c(c2)c2ccccc2n3-c2ccccc2)c2c(c1)c1c3ccccc3cc3c4c5c6cc(C(C)(C)C)cc7c8cc9ccccc9c(-c9ccc%10c(c9)c9ccccc9n%10-c9ccccc9)c8n(c5ncc4n2c31)c76]

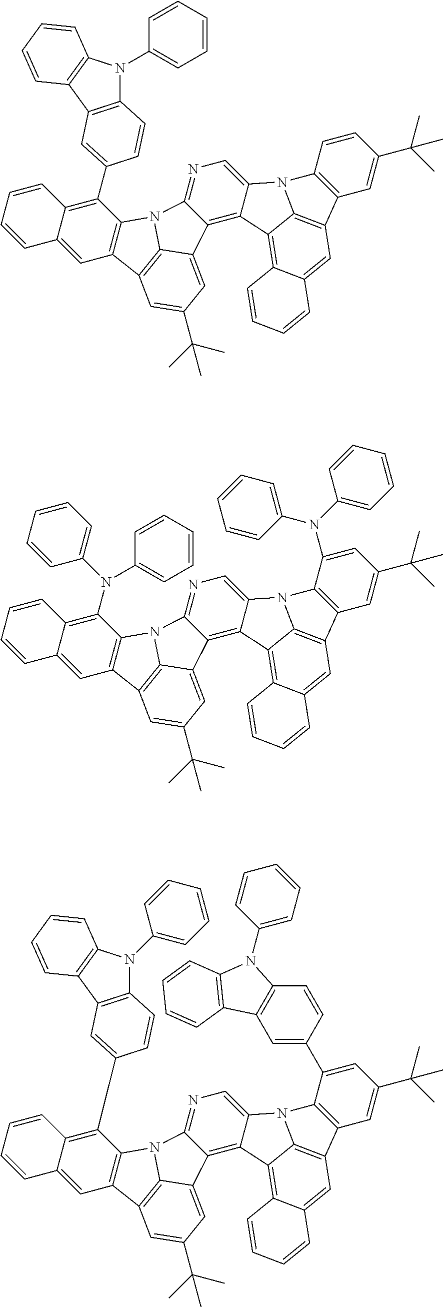 [SMILES: CC(C)(C)c1ccc2c(c1)c1cc3ccccc3c3c4c5c6cc(C(C)(C)C)cc7c8cc9ccccc9c(-c9ccc%10c(c9)c9ccccc9n%10-c9ccccc9)c8n(c5ncc4n2c13)c76 ; CC(C)(C)c1cc(N(c2ccccc2)c2ccccc2)c2c(c1)c1cc3ccccc3c3c4c5c6cc(C(C)(C)C)cc7c8cc9ccccc9c(N(c9ccccc9)c9ccccc9)c8n(c5ncc4n2c13)c76 ; CC(C)(C)c1cc(-c2ccc3c(c2)c2ccccc2n3-c2ccccc2)c2c(c1)c1cc3ccccc3c3c4c5c6cc(C(C)(C)C)cc7c8cc9ccccc9c(-c9ccc%10c(c9)c9ccccc9n%10-c9ccccc9)c8n(c5ncc4n2c13)c76]

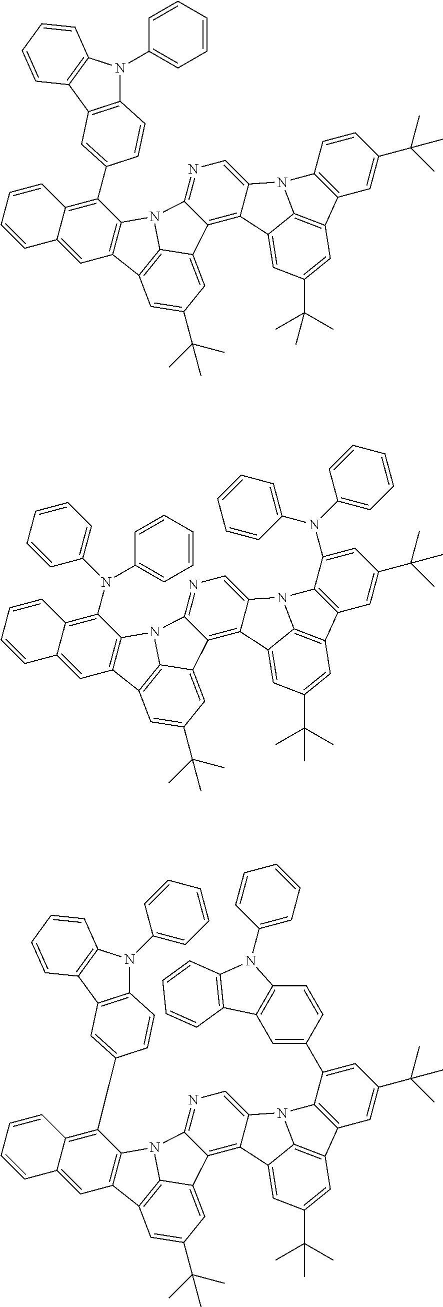 [SMILES: CC(C)(C)c1ccc2c(c1)c1cc(C(C)(C)C)cc3c4c5c6cc(C(C)(C)C)cc7c8cc9ccccc9c(-c9ccc%10c(c9)c9ccccc9n%10-c9ccccc9)c8n(c5ncc4n2c13)c76 ; CC(C)(C)c1cc(N(c2ccccc2)c2ccccc2)c2c(c1)c1cc(C(C)(C)C)cc3c4c5c6cc(C(C)(C)C)cc7c8cc9ccccc9c(N(c9ccccc9)c9ccccc9)c8n(c5ncc4n2c13)c76 ; CC(C)(C)c1cc(-c2ccc3c(c2)c2ccccc2n3-c2ccccc2)c2c(c1)c1cc(C(C)(C)C)cc3c4c5c6cc(C(C)(C)C)cc7c8cc9ccccc9c(-c9ccc%10c(c9)c9ccccc9n%10-c9ccccc9)c8n(c5ncc4n2c13)c76]

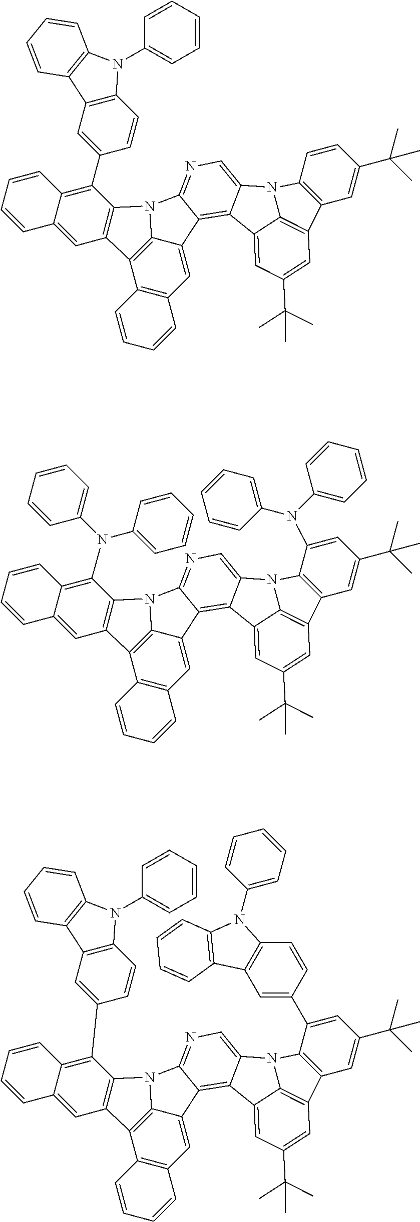 [SMILES: CC(C)(C)c1ccc2c(c1)c1cc(C(C)(C)C)cc3c4c5c6cc7ccccc7c7c8cc9ccccc9c(-c9ccc%10c(c9)c9ccccc9n%10-c9ccccc9)c8n(c5ncc4n2c13)c67 ; CC(C)(C)c1cc(N(c2ccccc2)c2ccccc2)c2c(c1)c1cc(C(C)(C)C)cc3c4c5c6cc7ccccc7c7c8cc9ccccc9c(N(c9ccccc9)c9ccccc9)c8n(c5ncc4n2c13)c67 ; CC(C)(C)c1cc(-c2ccc3c(c2)c2ccccc2n3-c2ccccc2)c2c(c1)c1cc(C(C)(C)C)cc3c4c5c6cc7ccccc7c7c8cc9ccccc9c(-c9ccc%10c(c9)c9ccccc9n%10-c9ccccc9)c8n(c5ncc4n2c13)c67]

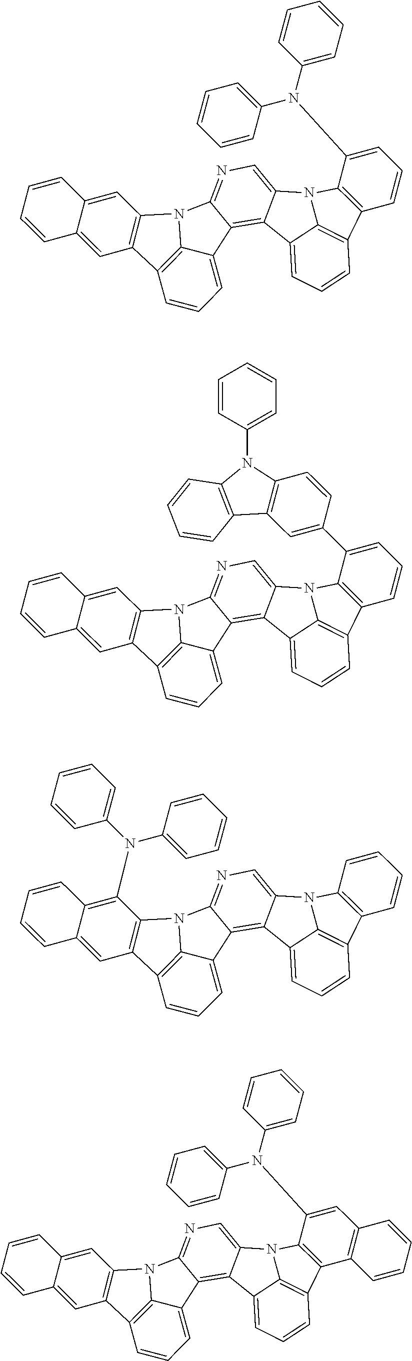 [SMILES: c1ccc(N(c2ccccc2)c2cccc3c4cccc5c6c7c8cccc9c%10cc%11ccccc%11cc%10n(c7ncc6n(c23)c45)c98)cc1 ; c1ccc(-n2c3ccccc3c3cc(-c4cccc5c6cccc7c8c9c%10cccc%11c%12cc%13ccccc%13cc%12n(c9ncc8n(c45)c67)c%11%10)ccc32)cc1 ; c1ccc(N(c2ccccc2)c2c3ccccc3cc3c4cccc5c6c7c8cccc9c%10ccccc%10n(c7cnc6n(c23)c45)c98)cc1 ; c1ccc(N(c2ccccc2)c2cc3ccccc3c3c4cccc5c6c7c8cccc9c%10cc%11ccccc%11cc%10n(c7ncc6n(c23)c54)c98)cc1]

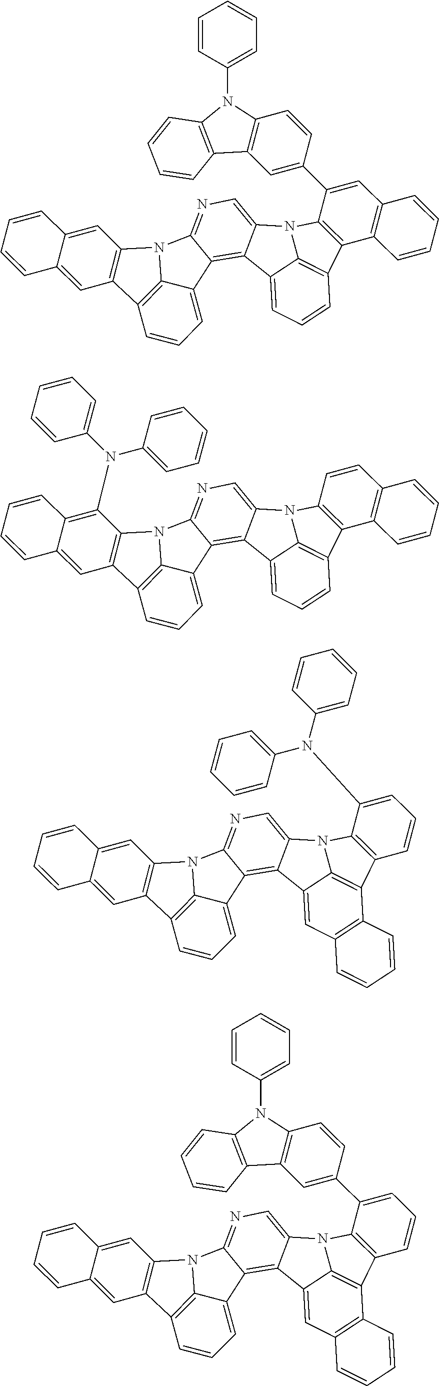 [SMILES: c1ccc(-n2c3ccccc3c3cc(-c4cc5ccccc5c5c6cccc7c8c9c%10cccc%11c%12cc%13ccccc%13cc%12n(c9ncc8n(c45)c76)c%11%10)ccc32)cc1 ; c1ccc(N(c2ccccc2)c2c3ccccc3cc3c4cccc5c6c7c8cccc9c%10c%11ccccc%11ccc%10n(c7cnc6n(c23)c45)c98)cc1 ; c1ccc(N(c2ccccc2)c2cccc3c4c5ccccc5cc5c6c7c8cccc9c%10cc%11ccccc%11cc%10n(c7ncc6n(c23)c54)c98)cc1 ; c1ccc(-n2c3ccccc3c3cc(-c4cccc5c6c7ccccc7cc7c8c9c%10cccc%11c%12cc%13ccccc%13cc%12n(c9ncc8n(c45)c76)c%11%10)ccc32)cc1]

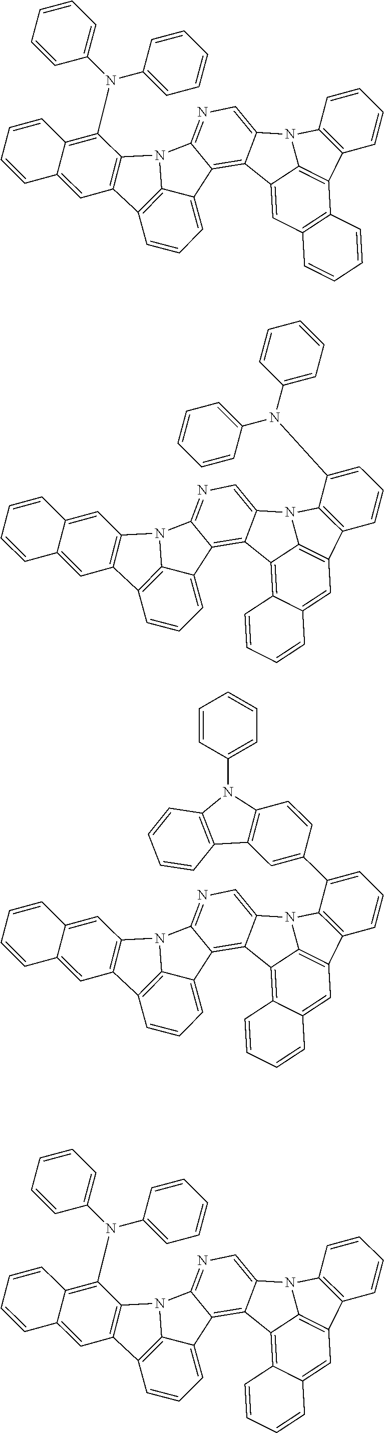 [SMILES: c1ccc(N(c2ccccc2)c2c3ccccc3cc3c4cccc5c6c7c8cc9ccccc9c9c%10ccccc%10n(c7cnc6n(c23)c45)c89)cc1 ; c1ccc(N(c2ccccc2)c2cccc3c4cc5ccccc5c5c6c7c8cccc9c%10cc%11ccccc%11cc%10n(c7ncc6n(c23)c45)c98)cc1 ; c1ccc(-n2c3ccccc3c3cc(-c4cccc5c6cc7ccccc7c7c8c9c%10cccc%11c%12cc%13ccccc%13cc%12n(c9ncc8n(c45)c67)c%11%10)ccc32)cc1 ; c1ccc(N(c2ccccc2)c2c3ccccc3cc3c4cccc5c6c7c8c9ccccc9cc9c%10ccccc%10n(c7cnc6n(c23)c45)c98)cc1]

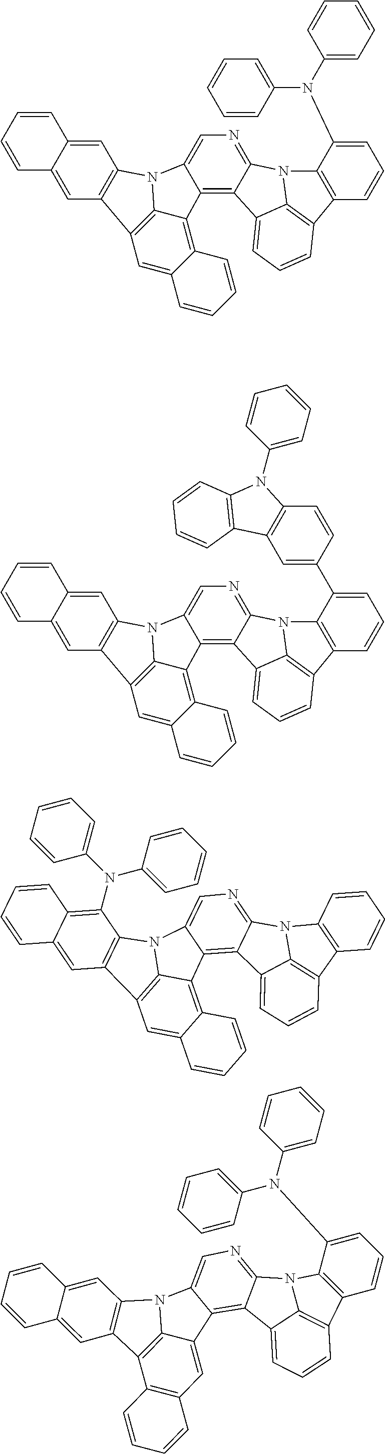 [SMILES: c1ccc(N(c2ccccc2)c2cccc3c4cccc5c6c7c8c9ccccc9cc9c%10cc%11ccccc%11cc%10n(c7cnc6n(c23)c45)c98)cc1 ; c1ccc(-n2c3ccccc3c3cc(-c4cccc5c6cccc7c8c9c%10c%11ccccc%11cc%11c%12cc%13ccccc%13cc%12n(c9cnc8n(c45)c67)c%11%10)ccc32)cc1 ; c1ccc(N(c2ccccc2)c2c3ccccc3cc3c4cc5ccccc5c5c6c7c8cccc9c%10ccccc%10n(c7ncc6n(c23)c45)c98)cc1 ; c1ccc(N(c2ccccc2)c2cccc3c4cccc5c6c7c8cc9ccccc9c9c%10cc%11ccccc%11cc%10n(c7cnc6n(c23)c45)c89)cc1]

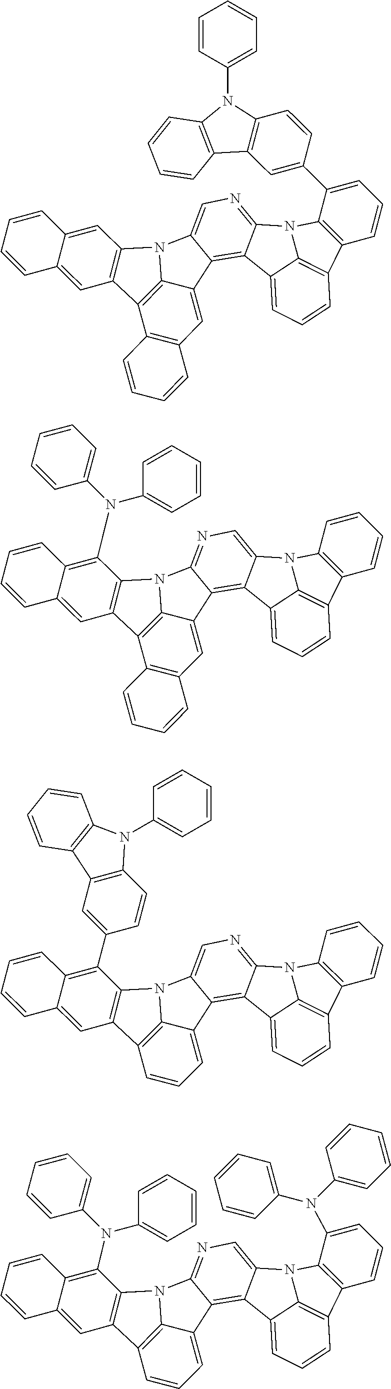

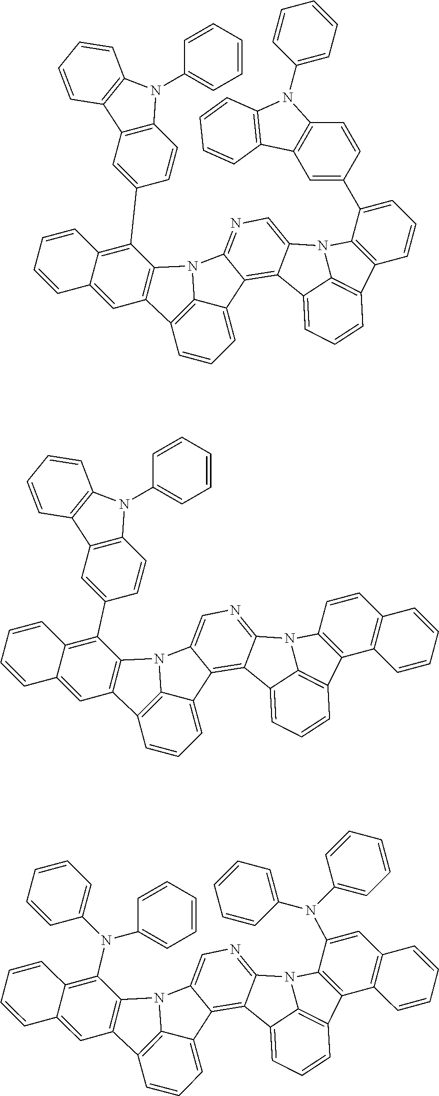 [SMILES: c1ccc(-n2c3ccccc3c3cc(-c4cccc5c6cccc7c8c9c%10cccc%11c%12cc%13ccccc%13c(-c%13ccc%14c(c%13)c%13ccccc%13n%14-c%13ccccc%13)c%12n(c9ncc8n(c45)c67)c%11%10)ccc32)cc1 ; c1ccc(-n2c3ccccc3c3cc(-c4c5ccccc5cc5c6cccc7c8c9c%10cccc%11c%12c%13ccccc%13ccc%12n(c9ncc8n(c45)c67)c%11%10)ccc32)cc1 ; c1ccc(N(c2ccccc2)c2c3ccccc3cc3c4cccc5c6c7c8cccc9c%10c%11ccccc%11cc(N(c%11ccccc%11)c%11ccccc%11)c%10n(c7ncc6n(c23)c45)c89)cc1]

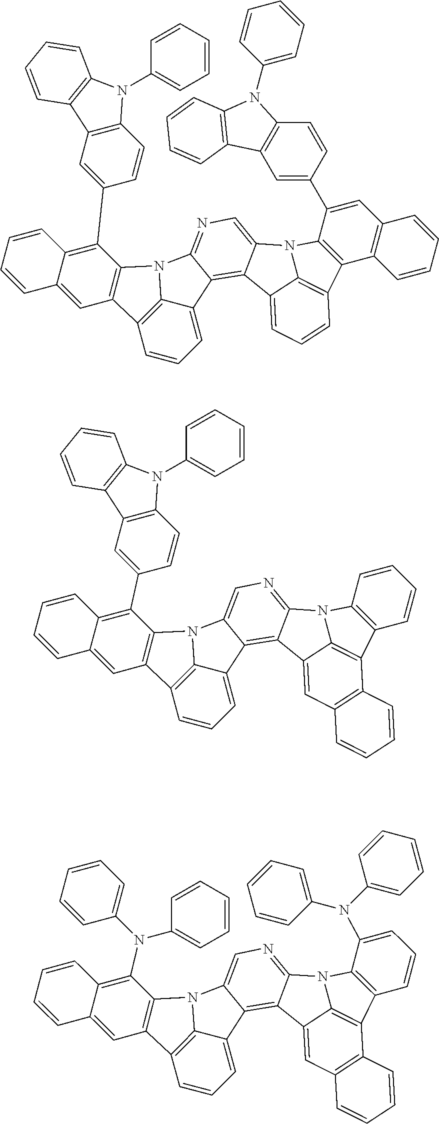 [SMILES: c1ccc(-n2c3ccccc3c3cc(-c4cc5ccccc5c5c6cccc7c8c9c%10cccc%11c%12cc%13ccccc%13c(-c%13ccc%14c(c%13)c%13ccccc%13n%14-c%13ccccc%13)c%12n(c9ncc8n(c45)c76)c%11%10)ccc32)cc1 ; c1ccc(-n2c3ccccc3c3cc(-c4c5ccccc5cc5c6cccc7c8c9c%10cc%11ccccc%11c%11c%12ccccc%12n(c9ncc8n(c45)c67)c%10%11)ccc32)cc1 ; c1ccc(N(c2ccccc2)c2c3ccccc3cc3c4cccc5c6c7c8cc9ccccc9c9c%10cccc(N(c%11ccccc%11)c%11ccccc%11)c%10n(c7ncc6n(c23)c45)c89)cc1]

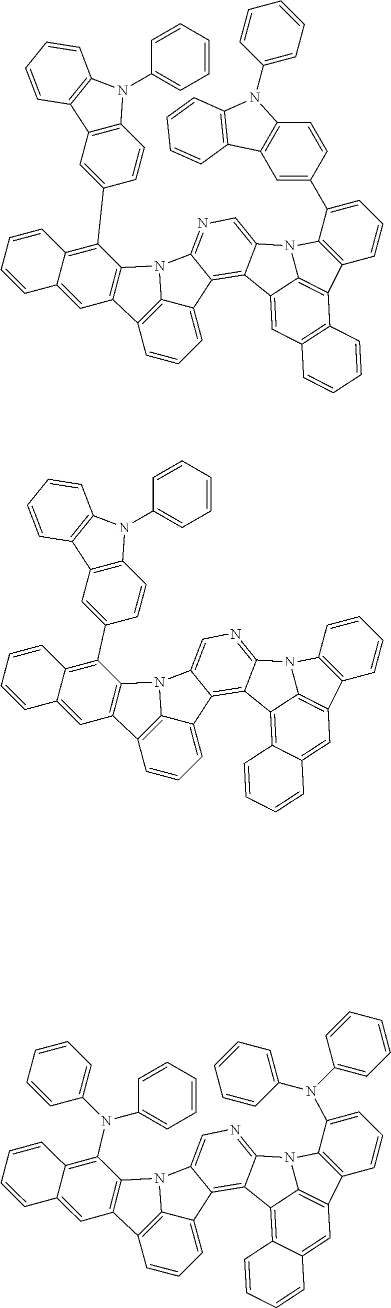 [SMILES: c1ccc(-n2c3ccccc3c3cc(-c4cccc5c6c7ccccc7cc7c8c9c%10cccc%11c%12cc%13ccccc%13c(-c%13ccc%14c(c%13)c%13ccccc%13n%14-c%13ccccc%13)c%12n(c9ncc8n(c45)c76)c%11%10)ccc32)cc1 ; c1ccc(-n2c3ccccc3c3cc(-c4c5ccccc5cc5c6cccc7c8c9c%10c%11ccccc%11cc%11c%12ccccc%12n(c9ncc8n(c45)c67)c%11%10)ccc32)cc1 ; c1ccc(N(c2ccccc2)c2c3ccccc3cc3c4cccc5c6c7c8c9ccccc9cc9c%10cccc(N(c%11ccccc%11)c%11ccccc%11)c%10n(c7ncc6n(c23)c45)c98)cc1]

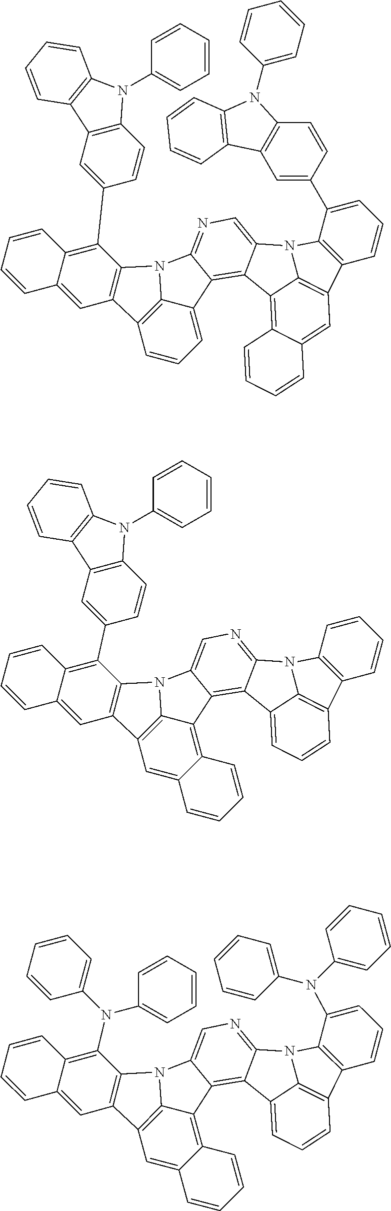 [SMILES: c1ccc(-n2c3ccccc3c3cc(-c4cccc5c6cc7ccccc7c7c8c9c%10cccc%11c%12cc%13ccccc%13c(-c%13ccc%14c(c%13)c%13ccccc%13n%14-c%13ccccc%13)c%12n(c9ncc8n(c45)c67)c%11%10)ccc32)cc1 ; c1ccc(-n2c3ccccc3c3cc(-c4c5ccccc5cc5c6cc7ccccc7c7c8c9c%10cccc%11c%12ccccc%12n(c9ncc8n(c45)c67)c%11%10)ccc32)cc1 ; c1ccc(N(c2ccccc2)c2c3ccccc3cc3c4cc5ccccc5c5c6c7c8cccc9c%10cccc(N(c%11ccccc%11)c%11ccccc%11)c%10n(c7ncc6n(c23)c45)c98)cc1]

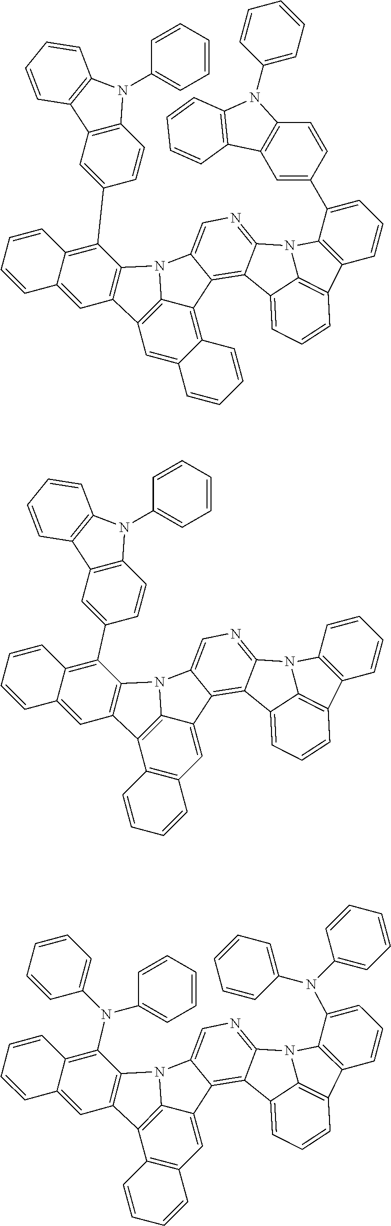 [SMILES: c1ccc(-n2c3ccccc3c3cc(-c4c5ccccc5cc5c6cc7ccccc7c7c8c9c%10cccc%11c%12cccc(-c%13ccc%14c(c%13)c%13ccccc%13n%14-c%13ccccc%13)c%12n(c9ncc8n(c45)c67)c%11%10)ccc32)cc1 ; c1ccc(-n2c3ccccc3c3cc(-c4c5ccccc5cc5c6c7ccccc7cc7c8c9c%10cccc%11c%12ccccc%12n(c9ncc8n(c45)c76)c%11%10)ccc32)cc1 ; c1ccc(N(c2ccccc2)c2c3ccccc3cc3c4c5ccccc5cc5c6c7c8cccc9c%10cccc(N(c%11ccccc%11)c%11ccccc%11)c%10n(c7ncc6n(c23)c54)c98)cc1]

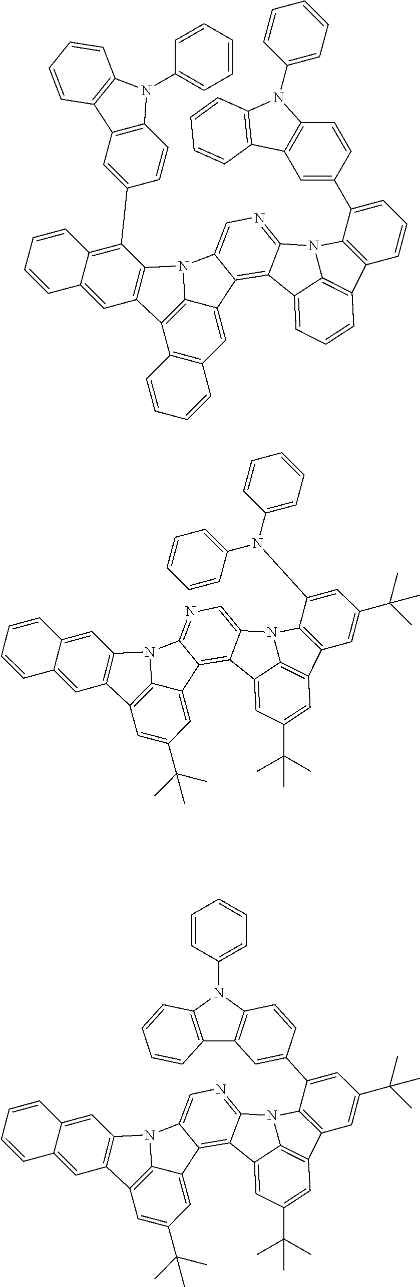 [SMILES: c1ccc(-n2c3ccccc3c3cc(-c4c5ccccc5cc5c6c7ccccc7cc7c8c9c%10cccc%11c%12cccc(-c%13ccc%14c(c%13)c%13ccccc%13n%14-c%13ccccc%13)c%12n(c9ncc8n(c45)c76)c%11%10)ccc32)cc1 ; CC(C)(C)c1cc(N(c2ccccc2)c2ccccc2)c2c(c1)c1cc(C(C)(C)C)cc3c4c5c6cc(C(C)(C)C)cc7c8cc9ccccc9cc8n(c5ncc4n2c13)c76 ; CC(C)(C)c1cc(-c2ccc3c(c2)c2ccccc2n3-c2ccccc2)c2c(c1)c1cc(C(C)(C)C)cc3c4c5c6cc(C(C)(C)C)cc7c8cc9ccccc9cc8n(c5cnc4n2c13)c76]

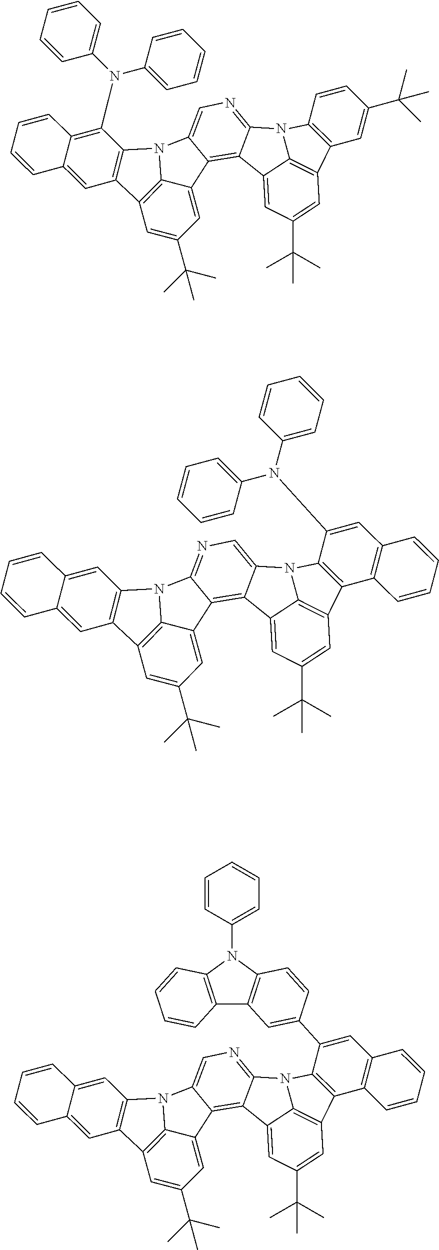 [SMILES: CC(C)(C)c1ccc2c(c1)c1cc(C(C)(C)C)cc3c4c5c6cc(C(C)(C)C)cc7c8cc9ccccc9c(N(c9ccccc9)c9ccccc9)c8n(c5cnc4n2c13)c76 ; CC(C)(C)c1cc2c3cc4ccccc4cc3n3c4ncc5c(c6cc(C(C)(C)C)cc7c8c9ccccc9cc(N(c9ccccc9)c9ccccc9)c8n5c67)c4c(c1)c23 ; CC(C)(C)c1cc2c3cc4ccccc4cc3n3c4cnc5c(c6cc(C(C)(C)C)cc7c8c9ccccc9cc(-c9ccc%10c(c9)c9ccccc9n%10-c9ccccc9)c8n5c67)c4c(c1)c23]

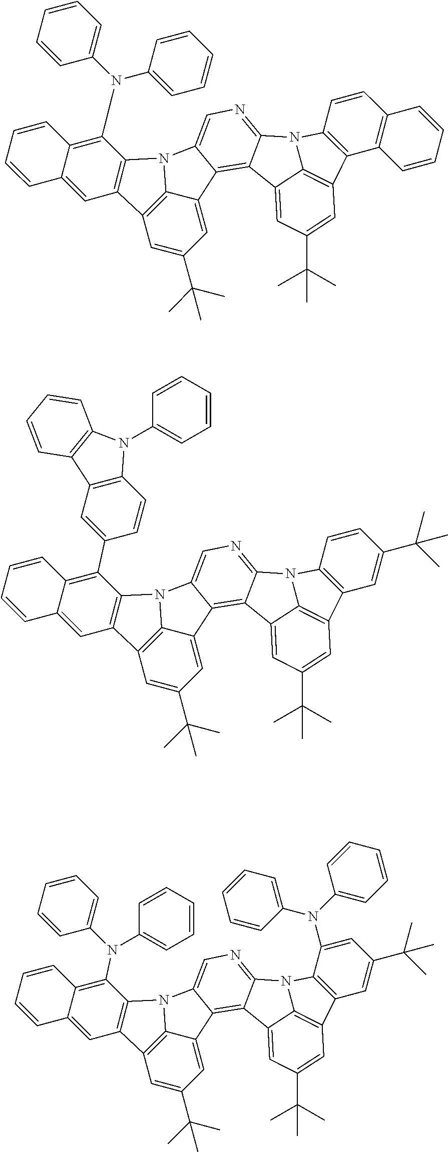 [SMILES: CC(C)(C)c1cc2c3c4ccccc4ccc3n3c4ncc5c(c6cc(C(C)(C)C)cc7c8cc9ccccc9c(N(c9ccccc9)c9ccccc9)c8n5c76)c4c(c1)c23 ; CC(C)(C)c1ccc2c(c1)c1cc(C(C)(C)C)cc3c4c5c6cc(C(C)(C)C)cc7c8cc9ccccc9c(-c9ccc%10c(c9)c9ccccc9n%10-c9ccccc9)c8n(c5cnc4n2c13)c76 ; CC(C)(C)c1cc(N(c2ccccc2)c2ccccc2)c2c(c1)c1cc(C(C)(C)C)cc3c4c5c6cc(C(C)(C)C)cc7c8cc9ccccc9c(N(c9ccccc9)c9ccccc9)c8n(c5cnc4n2c13)c76]

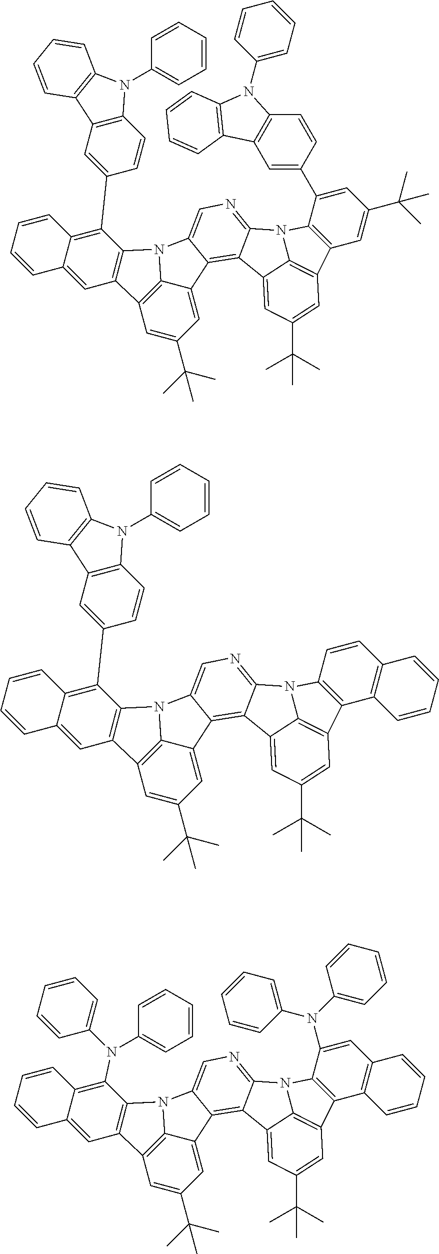 [SMILES: CC(C)(C)c1cc(-c2ccc3c(c2)c2ccccc2n3-c2ccccc2)c2c(c1)c1cc(C(C)(C)C)cc3c4c5c6cc(C(C)(C)C)cc7c8cc9ccccc9c(-c9ccc%10c(c9)c9ccccc9n%10-c9ccccc9)c8n(c5cnc4n2c13)c76 ; CC(C)(C)c1cc2c3c4ccccc4ccc3n3c4ncc5c(c6cc(C(C)(C)C)cc7c8cc9ccccc9c(-c9ccc%10c(c9)c9ccccc9n%10-c9ccccc9)c8n5c76)c4c(c1)c23 ; CC(C)(C)c1cc2c3cc4ccccc4c(N(c4ccccc4)c4ccccc4)c3n3c4cnc5c(c6cc(C(C)(C)C)cc7c8c9ccccc9cc(N(c9ccccc9)c9ccccc9)c8n5c67)c4c(c1)c23]

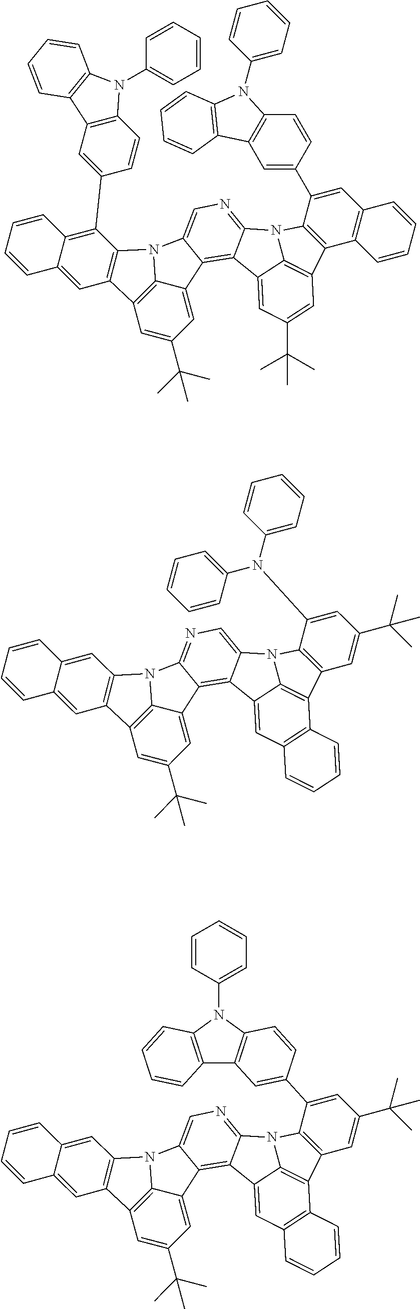 [SMILES: CC(C)(C)c1cc2c3cc4ccccc4c(-c4ccc5c(c4)c4ccccc4n5-c4ccccc4)c3n3c4cnc5c(c6cc(C(C)(C)C)cc7c8c9ccccc9cc(-c9ccc%10c(c9)c9ccccc9n%10-c9ccccc9)c8n5c67)c4c(c1)c23 ; CC(C)(C)c1cc(N(c2ccccc2)c2ccccc2)c2c(c1)c1c3ccccc3cc3c4c5c6cc(C(C)(C)C)cc7c8cc9ccccc9cc8n(c5ncc4n2c31)c76 ; CC(C)(C)c1cc(-c2ccc3c(c2)c2ccccc2n3-c2ccccc2)c2c(c1)c1c3ccccc3cc3c4c5c6cc(C(C)(C)C)cc7c8cc9ccccc9cc8n(c5cnc4n2c31)c76]

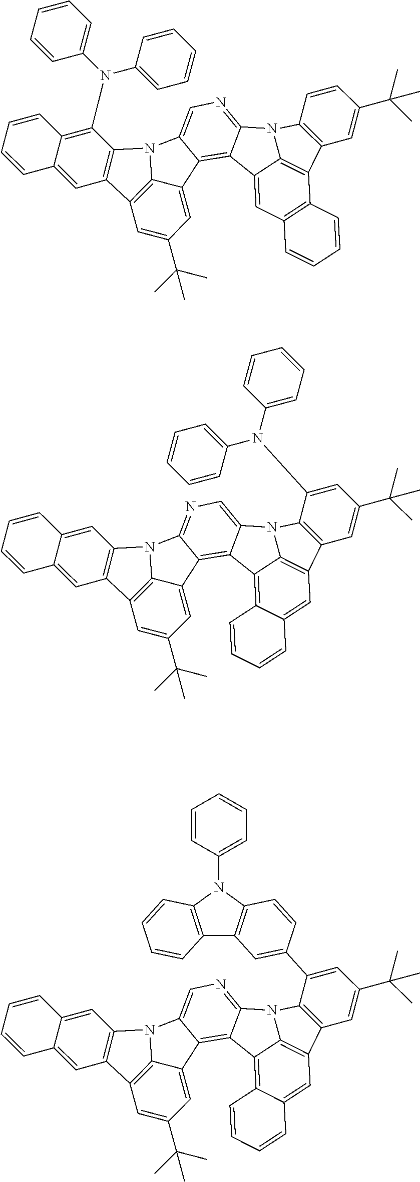 [SMILES: CC(C)(C)c1ccc2c(c1)c1c3ccccc3cc3c4c5c6cc(C(C)(C)C)cc7c8cc9ccccc9c(N(c9ccccc9)c9ccccc9)c8n(c5cnc4n2c31)c76 ; CC(C)(C)c1cc(N(c2ccccc2)c2ccccc2)c2c(c1)c1cc3ccccc3c3c4c5c6cc(C(C)(C)C)cc7c8cc9ccccc9cc8n(c5ncc4n2c13)c76 ; CC(C)(C)c1cc(-c2ccc3c(c2)c2ccccc2n3-c2ccccc2)c2c(c1)c1cc3ccccc3c3c4c5c6cc(C(C)(C)C)cc7c8cc9ccccc9cc8n(c5cnc4n2c13)c76]

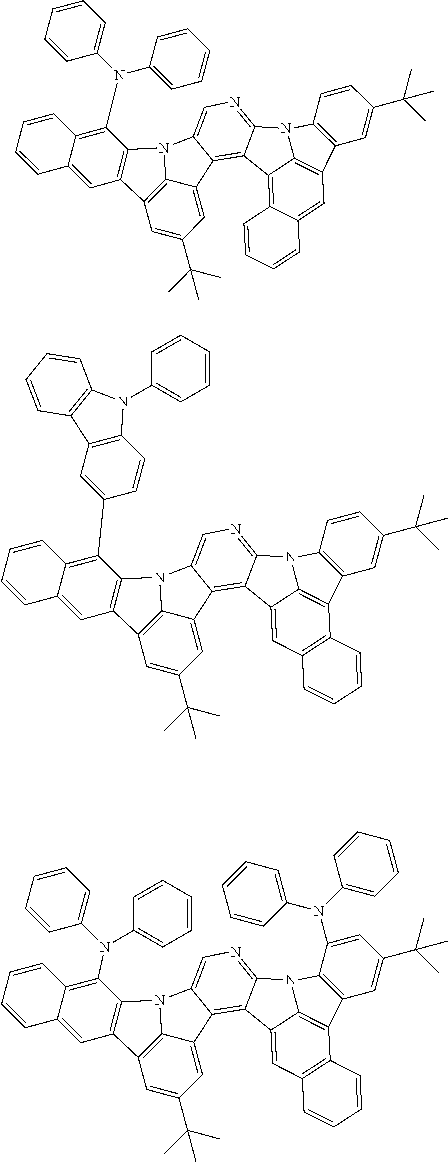

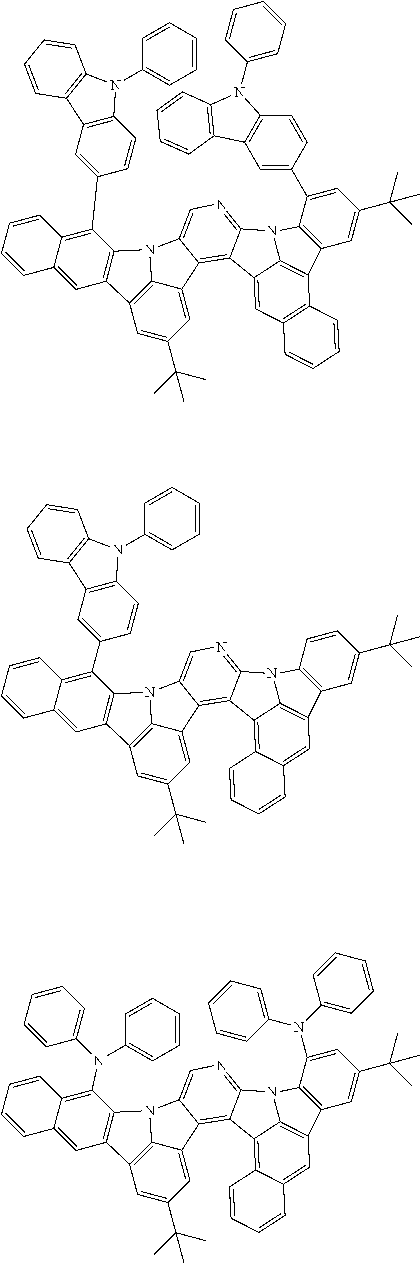 [SMILES: CC(C)(C)c1cc(-c2ccc3c(c2)c2ccccc2n3-c2ccccc2)c2c(c1)c1c3ccccc3cc3c4c5c6cc(C(C)(C)C)cc7c8cc9ccccc9c(-c9ccc%10c(c9)c9ccccc9n%10-c9ccccc9)c8n(c5cnc4n2c31)c76 ; CC(C)(C)c1ccc2c(c1)c1cc3ccccc3c3c4c5c6cc(C(C)(C)C)cc7c8cc9ccccc9c(-c9ccc%10c(c9)c9ccccc9n%10-c9ccccc9)c8n(c5cnc4n2c13)c76 ; CC(C)(C)c1cc(N(c2ccccc2)c2ccccc2)c2c(c1)c1cc3ccccc3c3c4c5c6cc(C(C)(C)C)cc7c8cc9ccccc9c(N(c9ccccc9)c9ccccc9)c8n(c5cnc4n2c13)c76]

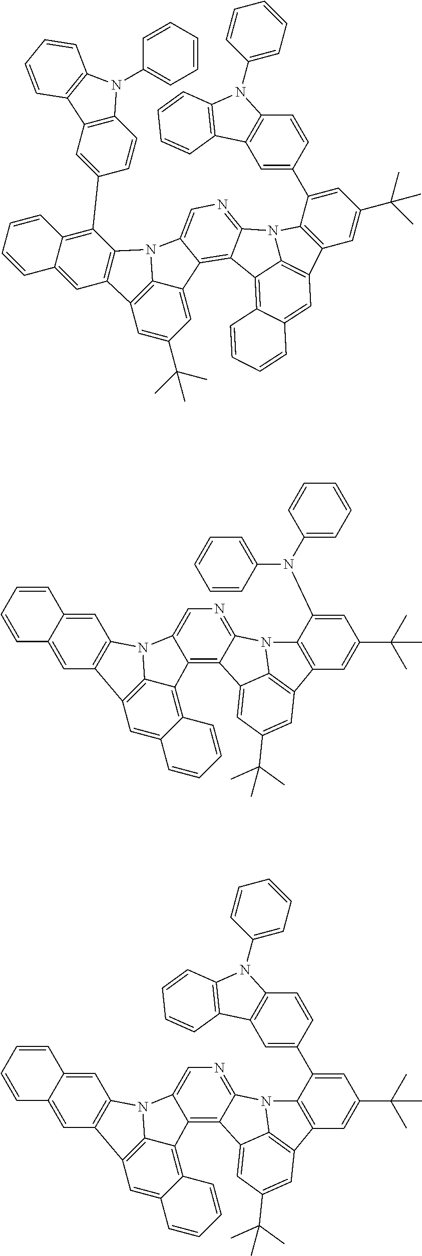 [SMILES: CC(C)(C)c1cc(-c2ccc3c(c2)c2ccccc2n3-c2ccccc2)c2c(c1)c1cc3ccccc3c3c4c5c6cc(C(C)(C)C)cc7c8cc9ccccc9c(-c9ccc%10c(c9)c9ccccc9n%10-c9ccccc9)c8n(c5cnc4n2c13)c76 ; CC(C)(C)c1cc(N(c2ccccc2)c2ccccc2)c2c(c1)c1cc(C(C)(C)C)cc3c4c5c6c7ccccc7cc7c8cc9ccccc9cc8n(c5cnc4n2c13)c76 ; CC(C)(C)c1cc(-c2ccc3c(c2)c2ccccc2n3-c2ccccc2)c2c(c1)c1cc(C(C)(C)C)cc3c4c5c6c7ccccc7cc7c8cc9ccccc9cc8n(c5cnc4n2c13)c76]

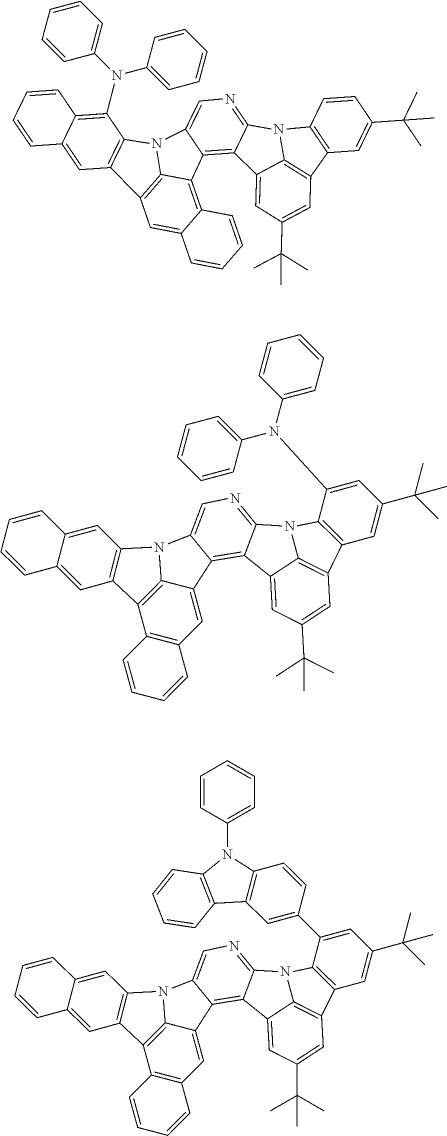 [SMILES: CC(C)(C)c1ccc2c(c1)c1cc(C(C)(C)C)cc3c4c5c6c7ccccc7cc7c8cc9ccccc9c(N(c9ccccc9)c9ccccc9)c8n(c5cnc4n2c13)c76 ; CC(C)(C)c1cc(N(c2ccccc2)c2ccccc2)c2c(c1)c1cc(C(C)(C)C)cc3c4c5c6cc7ccccc7c7c8cc9ccccc9cc8n(c5cnc4n2c13)c67 ; CC(C)(C)c1cc(-c2ccc3c(c2)c2ccccc2n3-c2ccccc2)c2c(c1)c1cc(C(C)(C)C)cc3c4c5c6cc7ccccc7c7c8cc9ccccc9cc8n(c5cnc4n2c13)c67]

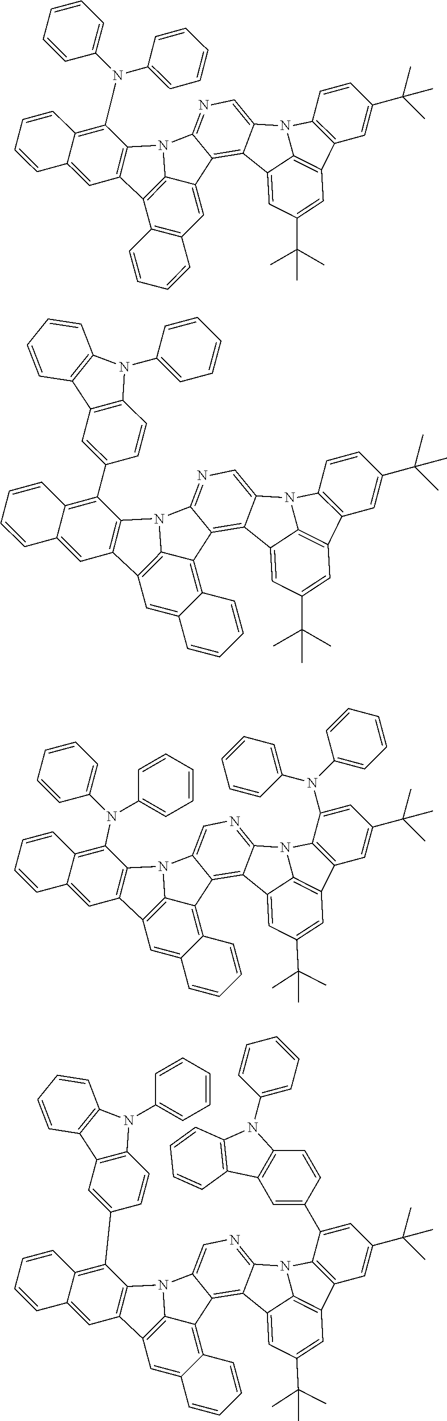

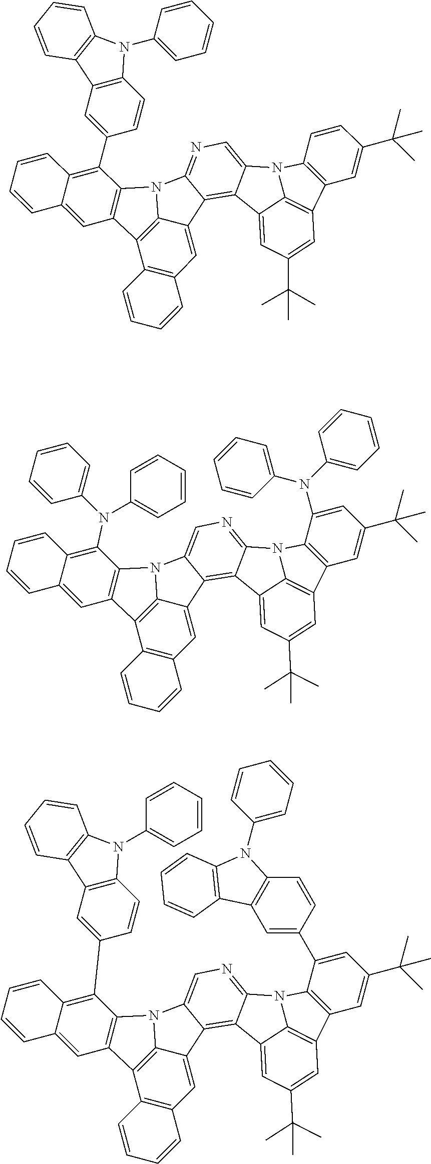 [SMILES: CC(C)(C)c1ccc2c(c1)c1cc(C(C)(C)C)cc3c4c5c6cc7ccccc7c7c8cc9ccccc9c(-c9ccc%10c(c9)c9ccccc9n%10-c9ccccc9)c8n(c5ncc4n2c13)c67 ; CC(C)(C)c1cc(N(c2ccccc2)c2ccccc2)c2c(c1)c1cc(C(C)(C)C)cc3c4c5c6cc7ccccc7c7c8cc9ccccc9c(N(c9ccccc9)c9ccccc9)c8n(c5cnc4n2c13)c67 ; CC(C)(C)c1cc(-c2ccc3c(c2)c2ccccc2n3-c2ccccc2)c2c(c1)c1cc(C(C)(C)C)cc3c4c5c6cc7ccccc7c7c8cc9ccccc9c(-c9ccc%10c(c9)c9ccccc9n%10-c9ccccc9)c8n(c5cnc4n2c13)c67]

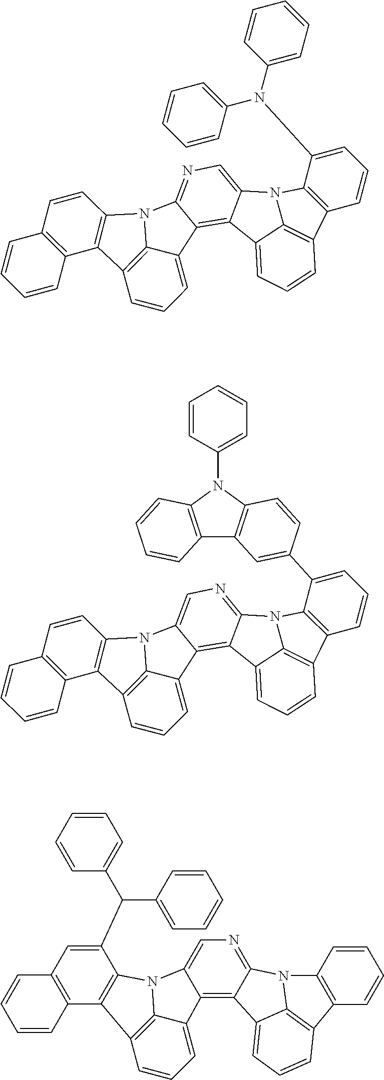 [SMILES: c1ccc(N(c2ccccc2)c2cccc3c4cccc5c6c7c8cccc9c%10c%11ccccc%11ccc%10n(c7ncc6n(c23)c45)c98)cc1 ; c1ccc(-n2c3ccccc3c3cc(-c4cccc5c6cccc7c8c9c%10cccc%11c%12c%13ccccc%13ccc%12n(c9cnc8n(c45)c67)c%11%10)ccc32)cc1 ; c1ccc(C(c2ccccc2)c2cc3ccccc3c3c4cccc5c6c7c8cccc9c%10ccccc%10n(c7ncc6n(c23)c54)c98)cc1]

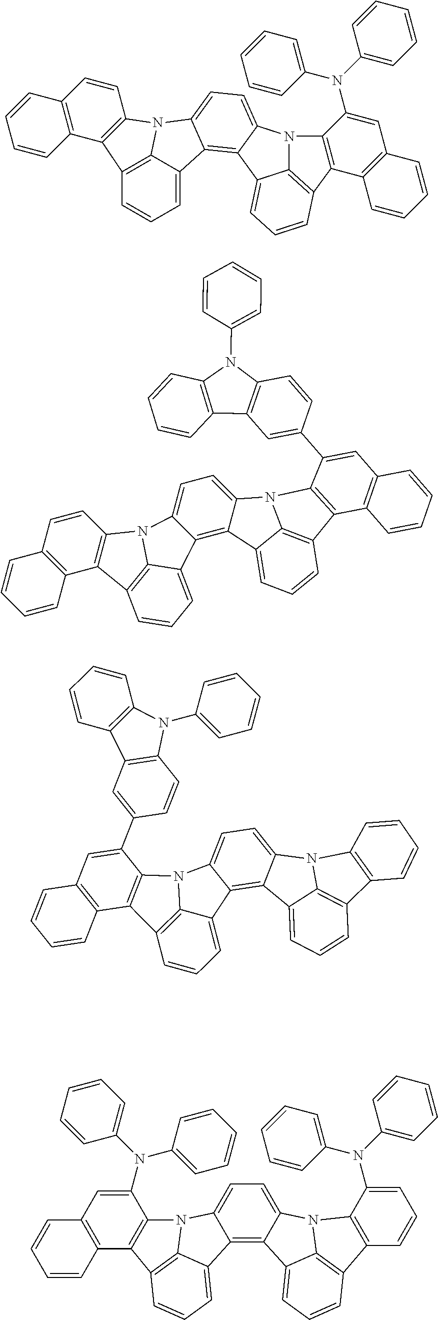

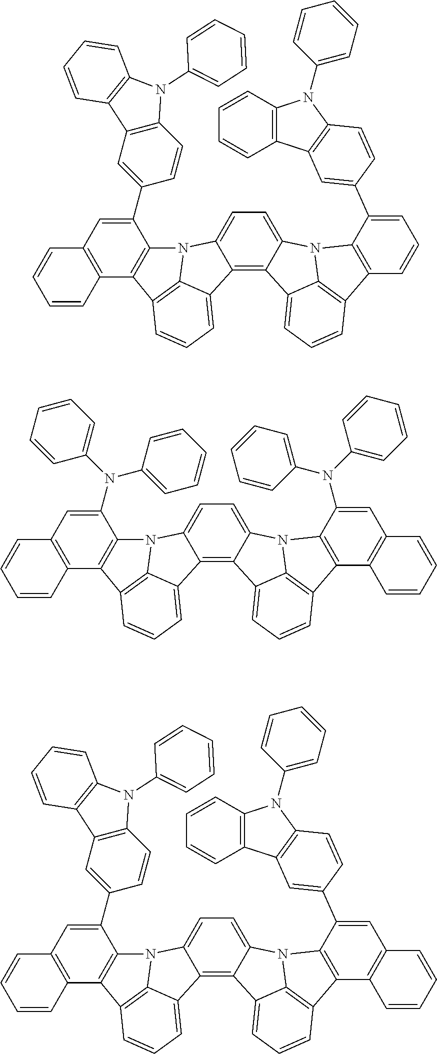 [SMILES: c1ccc(-n2c3ccccc3c3cc(-c4cccc5c6cccc7c8c9c%10cccc%11c%12c%13ccccc%13cc(-c%13ccc%14c(c%13)c%13ccccc%13n%14-c%13ccccc%13)c%12n(c9ccc8n(c45)c67)c%10%11)ccc32)cc1 ; c1ccc(N(c2ccccc2)c2cc3ccccc3c3c4cccc5c6c7c8cccc9c%10c%11ccccc%11cc(N(c%11ccccc%11)c%11ccccc%11)c%10n(c7ccc6n(c23)c54)c89)cc1 ; c1ccc(-n2c3ccccc3c3cc(-c4cc5ccccc5c5c6cccc7c8c9c%10cccc%11c%12c%13ccccc%13cc(-c%13ccc%14c(c%13)c%13ccccc%13n%14-c%13ccccc%13)c%12n(c9ccc8n(c45)c76)c%10%11)ccc32)cc1]

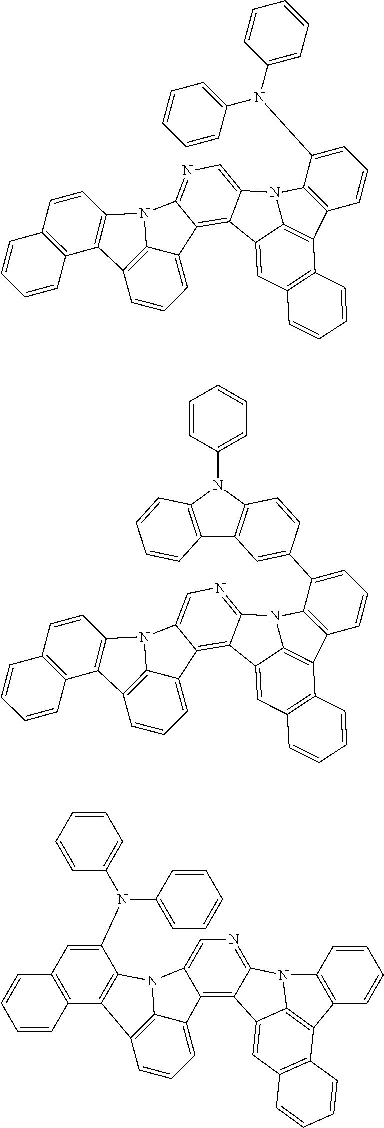 [SMILES: c1ccc(N(c2ccccc2)c2cccc3c4c5ccccc5cc5c6c7c8cccc9c%10c%11ccccc%11ccc%10n(c7ncc6n(c23)c54)c98)cc1 ; c1ccc(-n2c3ccccc3c3cc(-c4cccc5c6c7ccccc7cc7c8c9c%10cccc%11c%12c%13ccccc%13ccc%12n(c9cnc8n(c45)c76)c%11%10)ccc32)cc1 ; c1ccc(N(c2ccccc2)c2cc3ccccc3c3c4cccc5c6c7c8cc9ccccc9c9c%10ccccc%10n(c7ncc6n(c23)c54)c89)cc1]

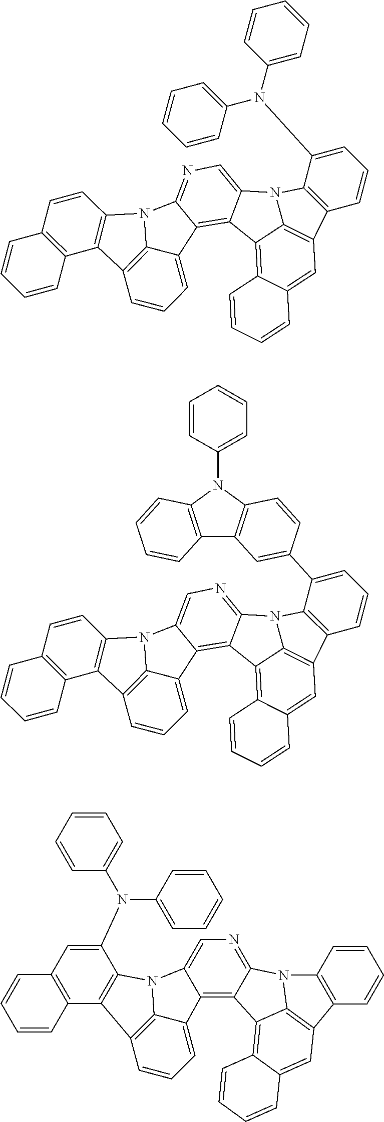 [SMILES: c1ccc(N(c2ccccc2)c2cccc3c4cc5ccccc5c5c6c7c8cccc9c%10c%11ccccc%11ccc%10n(c7ncc6n(c23)c45)c98)cc1 ; c1ccc(-n2c3ccccc3c3cc(-c4cccc5c6cc7ccccc7c7c8c9c%10cccc%11c%12c%13ccccc%13ccc%12n(c9cnc8n(c45)c67)c%11%10)ccc32)cc1 ; c1ccc(N(c2ccccc2)c2cc3ccccc3c3c4cccc5c6c7c8c9ccccc9cc9c%10ccccc%10n(c7ncc6n(c23)c54)c98)cc1]

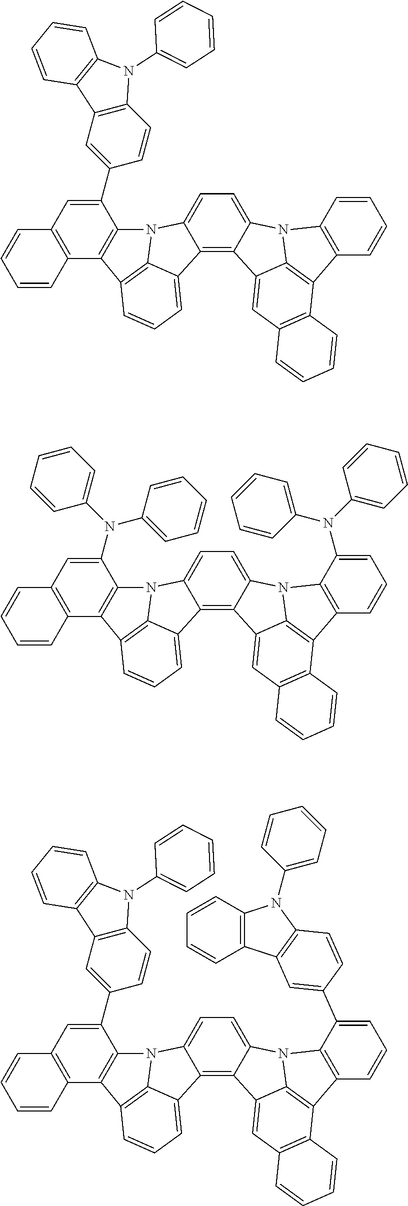 [SMILES: c1ccc(-n2c3ccccc3c3cc(-c4cc5ccccc5c5c6cccc7c8c9c%10cc%11ccccc%11c%11c%12ccccc%12n(c9ccc8n(c45)c76)c%10%11)ccc32)cc1 ; c1ccc(N(c2ccccc2)c2cc3ccccc3c3c4cccc5c6c7c8cc9ccccc9c9c%10cccc(N(c%11ccccc%11)c%11ccccc%11)c%10n(c7ccc6n(c23)c54)c89)cc1 ; c1ccc(-n2c3ccccc3c3cc(-c4cc5ccccc5c5c6cccc7c8c9c%10cc%11ccccc%11c%11c%12cccc(-c%13ccc%14c(c%13)c%13ccccc%13n%14-c%13ccccc%13)c%12n(c9ccc8n(c45)c76)c%10%11)ccc32)cc1]

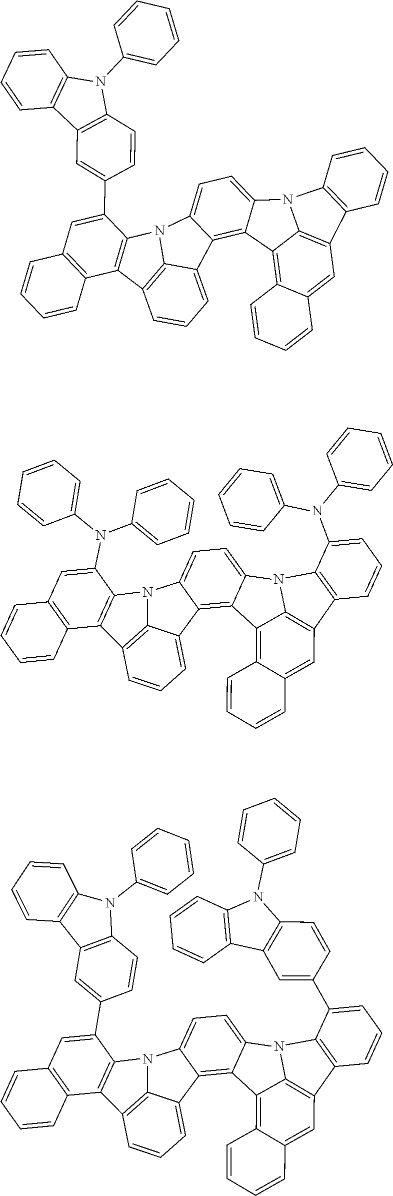 [SMILES: c1ccc(-n2c3ccccc3c3cc(-c4cc5ccccc5c5c6cccc7c8c9c%10c%11ccccc%11cc%11c%12ccccc%12n(c9ccc8n(c45)c76)c%11%10)ccc32)cc1 ; c1ccc(N(c2ccccc2)c2cc3ccccc3c3c4cccc5c6c7c8c9ccccc9cc9c%10cccc(N(c%11ccccc%11)c%11ccccc%11)c%10n(c7ccc6n(c23)c54)c98)cc1 ; c1ccc(-n2c3ccccc3c3cc(-c4cc5ccccc5c5c6cccc7c8c9c%10c%11ccccc%11cc%11c%12cccc(-c%13ccc%14c(c%13)c%13ccccc%13n%14-c%13ccccc%13)c%12n(c9ccc8n(c45)c76)c%11%10)ccc32)cc1]

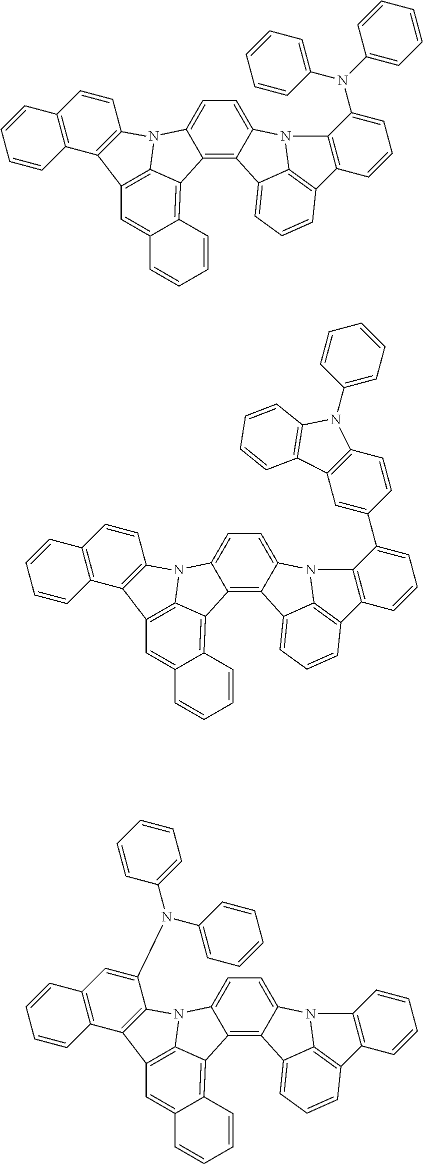 [SMILES: c1ccc(N(c2ccccc2)c2cccc3c4cccc5c6c7c8c9ccccc9cc9c%10c%11ccccc%11ccc%10n(c7ccc6n(c23)c45)c98)cc1 ; c1ccc(-n2c3ccccc3c3cc(-c4cccc5c6cccc7c8c9c%10c%11ccccc%11cc%11c%12c%13ccccc%13ccc%12n(c9ccc8n(c45)c67)c%11%10)ccc32)cc1 ; c1ccc(N(c2ccccc2)c2cc3ccccc3c3c4cc5ccccc5c5c6c7c8cccc9c%10ccccc%10n(c7ccc6n(c23)c45)c98)cc1]

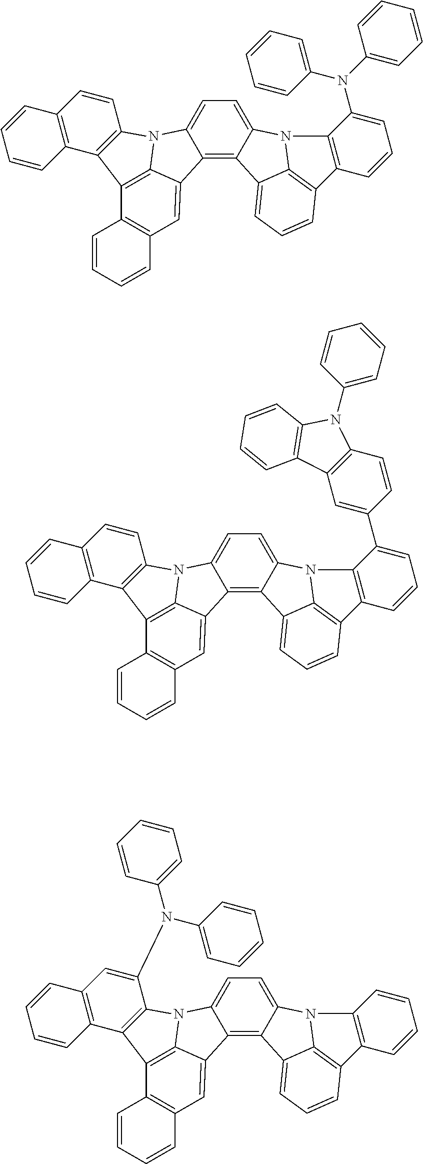 [SMILES: c1ccc(N(c2ccccc2)c2cccc3c4cccc5c6c7c8cc9ccccc9c9c%10c%11ccccc%11ccc%10n(c7ccc6n(c23)c45)c89)cc1 ; c1ccc(-n2c3ccccc3c3cc(-c4cccc5c6cccc7c8c9c%10cc%11ccccc%11c%11c%12c%13ccccc%13ccc%12n(c9ccc8n(c45)c67)c%10%11)ccc32)cc1 ; c1ccc(N(c2ccccc2)c2cc3ccccc3c3c4c5ccccc5cc5c6c7c8cccc9c%10ccccc%10n(c7ccc6n(c23)c54)c98)cc1]

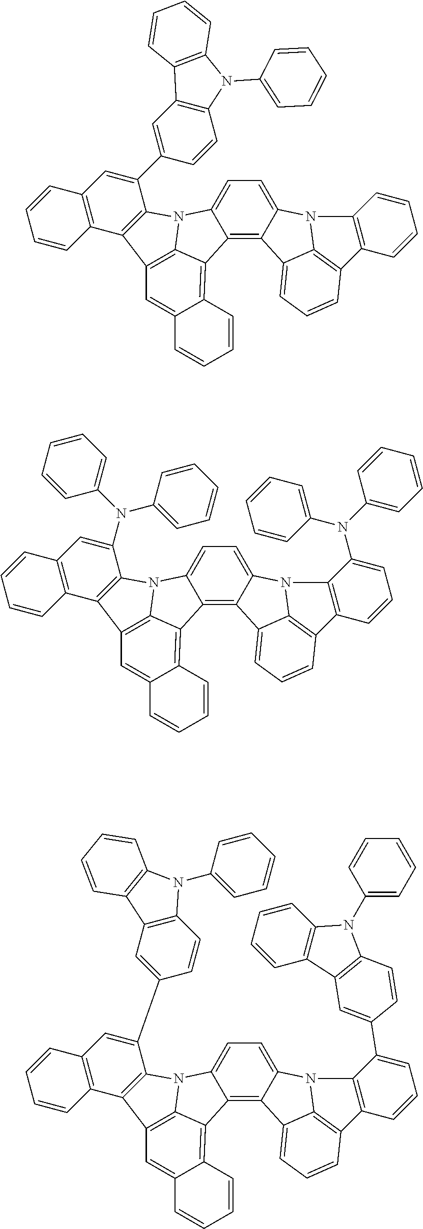 [SMILES: c1ccc(-n2c3ccccc3c3cc(-c4cc5ccccc5c5c6cc7ccccc7c7c8c9c%10cccc%11c%12ccccc%12n(c9ccc8n(c45)c67)c%11%10)ccc32)cc1 ; c1ccc(N(c2ccccc2)c2cccc3c4cccc5c6c7c8c9ccccc9cc9c%10c%11ccccc%11cc(N(c%11ccccc%11)c%11ccccc%11)c%10n(c7ccc6n(c23)c45)c98)cc1 ; c1ccc(-n2c3ccccc3c3cc(-c4cccc5c6cccc7c8c9c%10c%11ccccc%11cc%11c%12c%13ccccc%13cc(-c%13ccc%14c(c%13)c%13ccccc%13n%14-c%13ccccc%13)c%12n(c9ccc8n(c45)c67)c%11%10)ccc32)cc1]

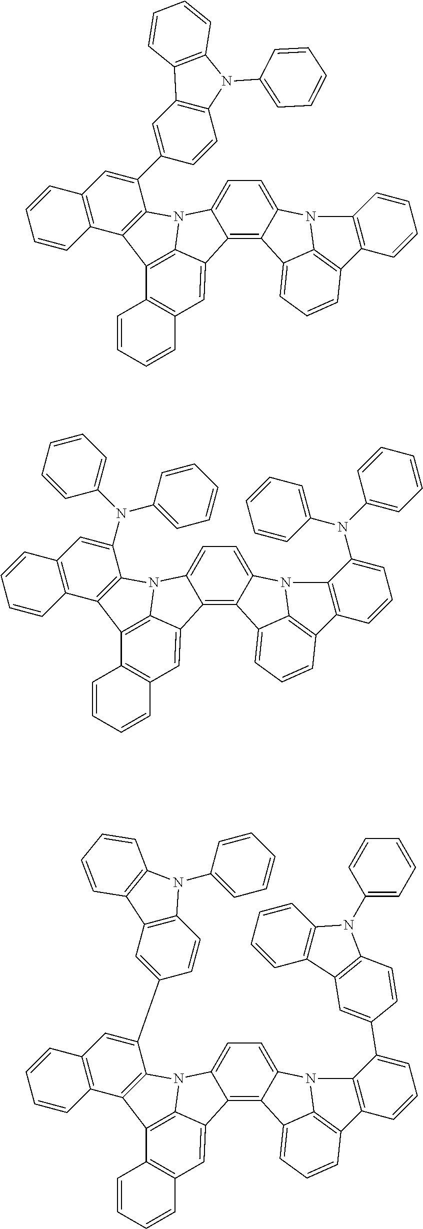 [SMILES: c1ccc(-n2c3ccccc3c3cc(-c4cc5ccccc5c5c6c7ccccc7cc7c8c9c%10cccc%11c%12ccccc%12n(c9ccc8n(c45)c76)c%11%10)ccc32)cc1 ; c1ccc(N(c2ccccc2)c2cccc3c4cccc5c6c7c8cc9ccccc9c9c%10c%11ccccc%11cc(N(c%11ccccc%11)c%11ccccc%11)c%10n(c7ccc6n(c23)c45)c89)cc1 ; c1ccc(-n2c3ccccc3c3cc(-c4cccc5c6cccc7c8c9c%10cc%11ccccc%11c%11c%12c%13ccccc%13cc(-c%13ccc%14c(c%13)c%13ccccc%13n%14-c%13ccccc%13)c%12n(c9ccc8n(c45)c67)c%10%11)ccc32)cc1]

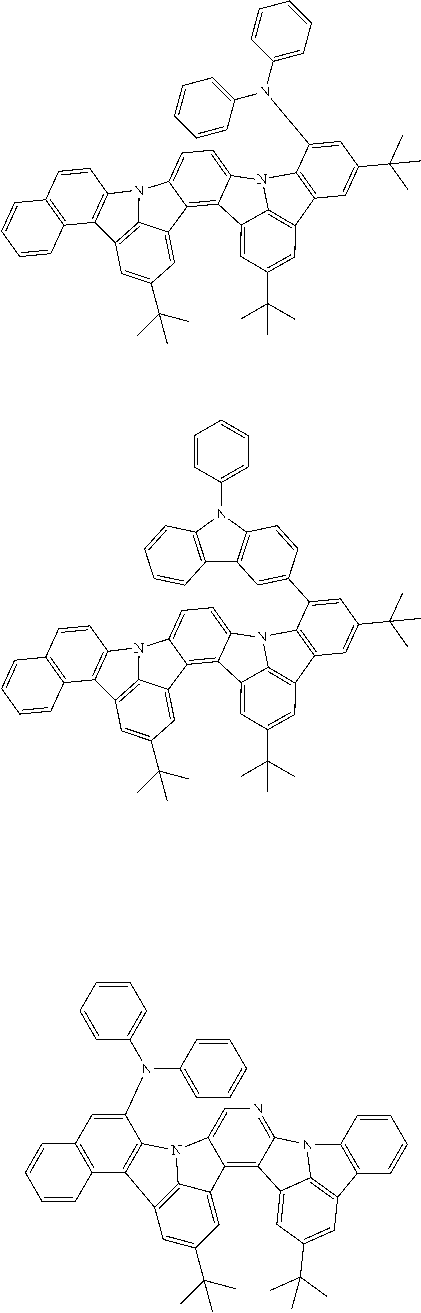 [SMILES: CC(C)(C)c1cc(N(c2ccccc2)c2ccccc2)c2c(c1)c1cc(C(C)(C)C)cc3c4c5c6cc(C(C)(C)C)cc7c8c9ccccc9ccc8n(c5ccc4n2c13)c76 ; CC(C)(C)c1cc(-c2ccc3c(c2)c2ccccc2n3-c2ccccc2)c2c(c1)c1cc(C(C)(C)C)cc3c4c5c6cc(C(C)(C)C)cc7c8c9ccccc9ccc8n(c5ccc4n2c13)c76 ; CC(C)(C)c1cc2c3ccccc3n3c4ncc5c(c6cc(C(C)(C)C)cc7c8c9ccccc9cc(N(c9ccccc9)c9ccccc9)c8n5c67)c4c(c1)c23]

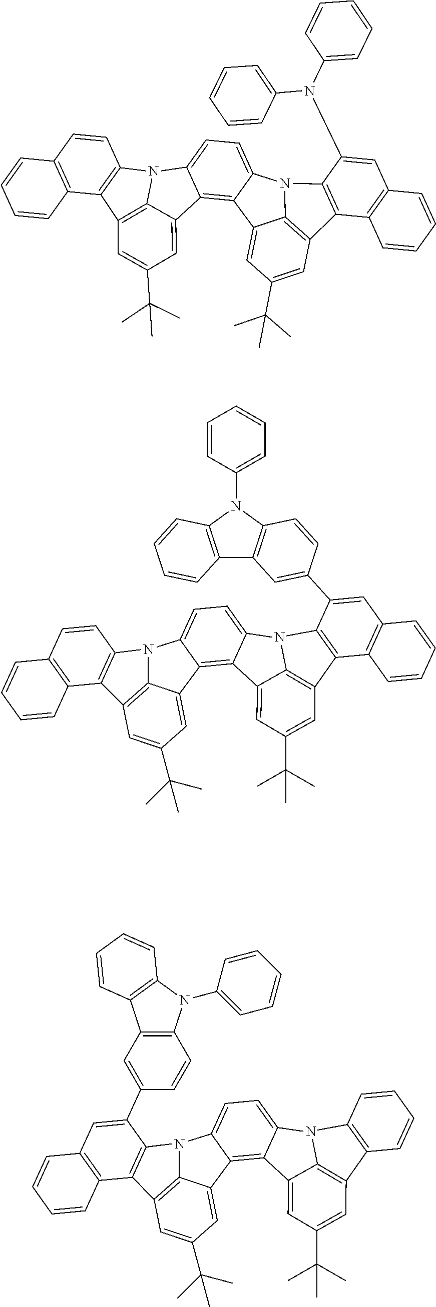 [SMILES: CC(C)(C)c1cc2c3c4ccccc4ccc3n3c4ccc5c(c6cc(C(C)(C)C)cc7c8c9ccccc9cc(N(c9ccccc9)c9ccccc9)c8n5c67)c4c(c1)c23 ; CC(C)(C)c1cc2c3c4ccccc4ccc3n3c4ccc5c(c6cc(C(C)(C)C)cc7c8c9ccccc9cc(-c9ccc%10c(c9)c9ccccc9n%10-c9ccccc9)c8n5c67)c4c(c1)c23 ; CC(C)(C)c1cc2c3ccccc3n3c4ccc5c(c6cc(C(C)(C)C)cc7c8c9ccccc9cc(-c9ccc%10c(c9)c9ccccc9n%10-c9ccccc9)c8n5c67)c4c(c1)c23]

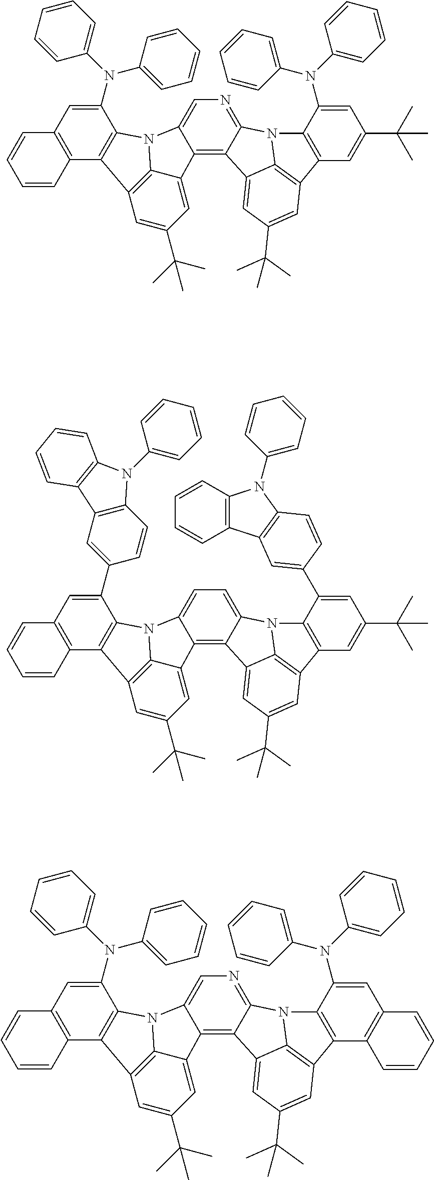 [SMILES: CC(C)(C)c1cc(N(c2ccccc2)c2ccccc2)c2c(c1)c1cc(C(C)(C)C)cc3c4c5c6cc(C(C)(C)C)cc7c8c9ccccc9cc(N(c9ccccc9)c9ccccc9)c8n(c5cnc4n2c13)c67 ; CC(C)(C)c1cc(-c2ccc3c(c2)c2ccccc2n3-c2ccccc2)c2c(c1)c1cc(C(C)(C)C)cc3c4c5c6cc(C(C)(C)C)cc7c8c9ccccc9cc(-c9ccc%10c(c9)c9ccccc9n%10-c9ccccc9)c8n(c5ccc4n2c13)c67 ; CC(C)(C)c1cc2c3c4c5cc(C(C)(C)C)cc6c7c8ccccc8cc(N(c8ccccc8)c8ccccc8)c7n(c4ncc3n3c4c(N(c7ccccc7)c7ccccc7)cc7ccccc7c4c(c1)c23)c56]

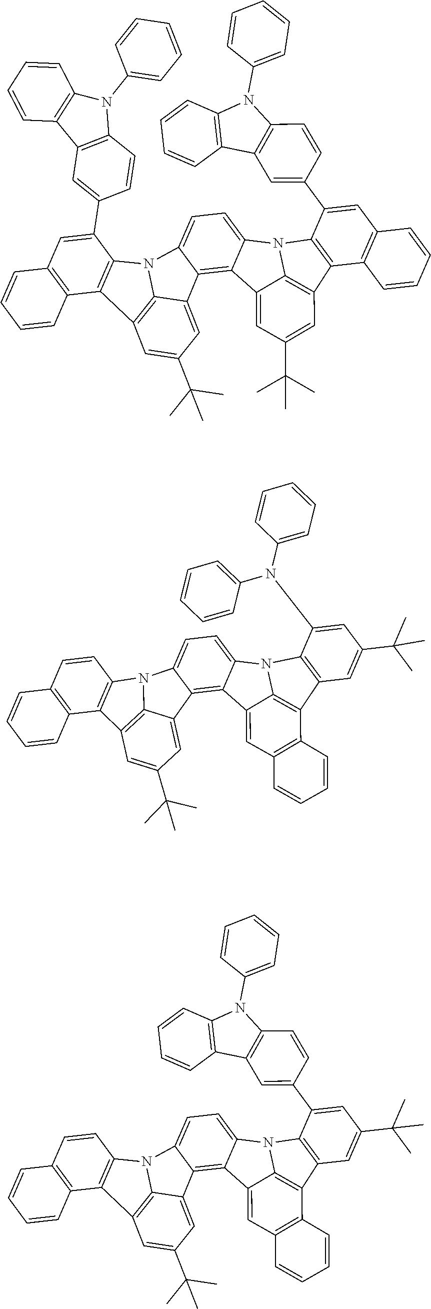 [SMILES: CC(C)(C)c1cc2c3c4c5cc(C(C)(C)C)cc6c7c8ccccc8cc(-c8ccc9c(c8)c8ccccc8n9-c8ccccc8)c7n(c4ccc3n3c4c(-c7ccc8c(c7)c7ccccc7n8-c7ccccc7)cc7ccccc7c4c(c1)c23)c56 ; CC(C)(C)c1cc(N(c2ccccc2)c2ccccc2)c2c(c1)c1c3ccccc3cc3c4c5c6cc(C(C)(C)C)cc7c8c9ccccc9ccc8n(c5ccc4n2c31)c76 ; CC(C)(C)c1cc(-c2ccc3c(c2)c2ccccc2n3-c2ccccc2)c2c(c1)c1c3ccccc3cc3c4c5c6cc(C(C)(C)C)cc7c8c9ccccc9ccc8n(c5ccc4n2c31)c76]

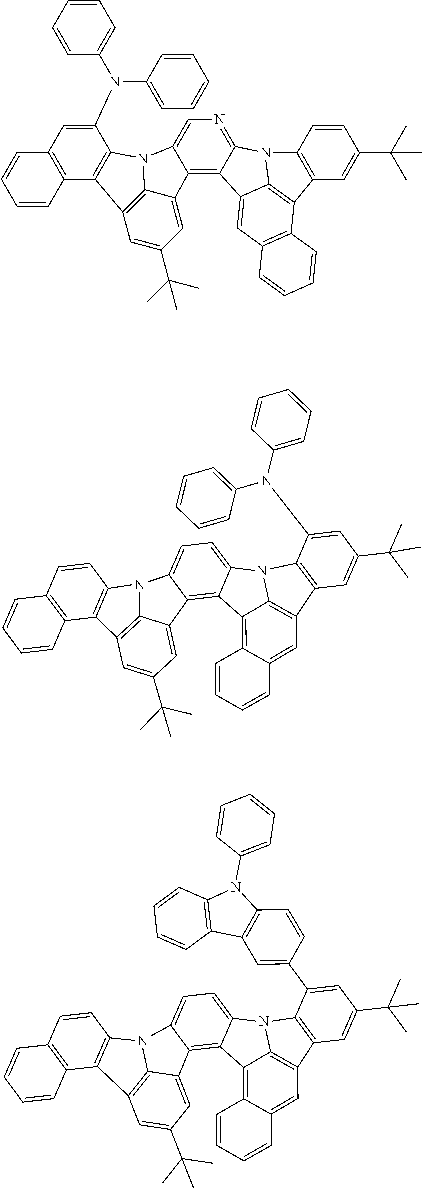 [SMILES: CC(C)(C)c1ccc2c(c1)c1c3ccccc3cc3c4c5c6cc(C(C)(C)C)cc7c8c9ccccc9cc(N(c9ccccc9)c9ccccc9)c8n(c5cnc4n2c31)c67 ; CC(C)(C)c1cc(N(c2ccccc2)c2ccccc2)c2c(c1)c1cc3ccccc3c3c4c5c6cc(C(C)(C)C)cc7c8c9ccccc9ccc8n(c5ccc4n2c13)c76 ; CC(C)(C)c1cc(-c2ccc3c(c2)c2ccccc2n3-c2ccccc2)c2c(c1)c1cc3ccccc3c3c4c5c6cc(C(C)(C)C)cc7c8c9ccccc9ccc8n(c5ccc4n2c13)c76]

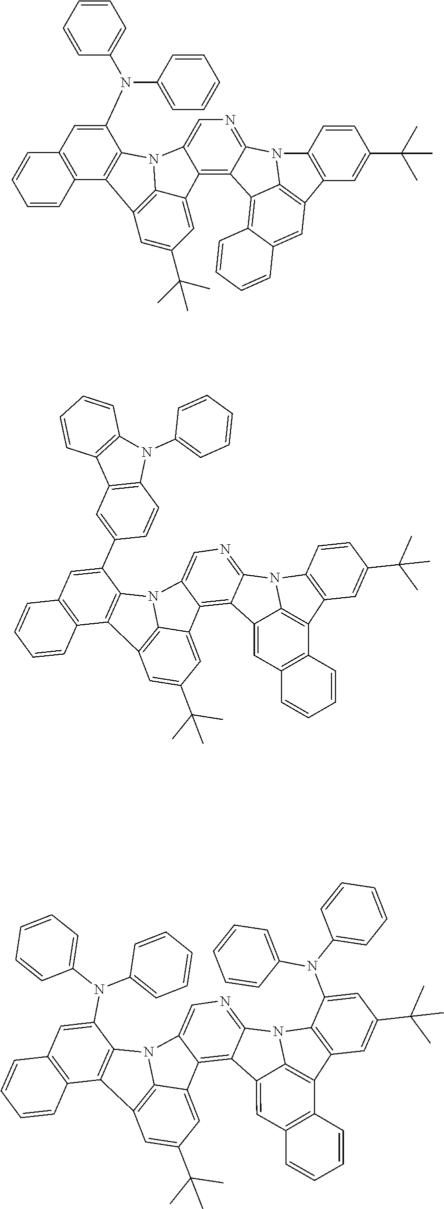 [SMILES: CC(C)(C)c1ccc2c(c1)c1cc3ccccc3c3c4c5c6cc(C(C)(C)C)cc7c8c9ccccc9cc(N(c9ccccc9)c9ccccc9)c8n(c5cnc4n2c13)c67 ; CC(C)(C)c1ccc2c(c1)c1c3ccccc3cc3c4c5c6cc(C(C)(C)C)cc7c8c9ccccc9cc(-c9ccc%10c(c9)c9ccccc9n%10-c9ccccc9)c8n(c5cnc4n2c31)c67 ; CC(C)(C)c1cc(N(c2ccccc2)c2ccccc2)c2c(c1)c1c3ccccc3cc3c4c5c6cc(C(C)(C)C)cc7c8c9ccccc9cc(N(c9ccccc9)c9ccccc9)c8n(c5cnc4n2c31)c67]

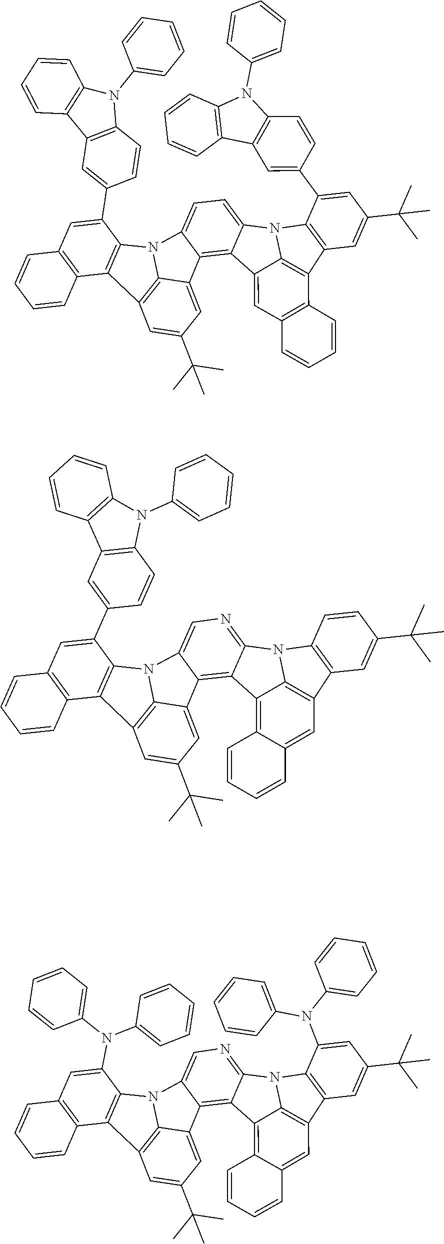 [SMILES: CC(C)(C)c1cc(-c2ccc3c(c2)c2ccccc2n3-c2ccccc2)c2c(c1)c1c3ccccc3cc3c4c5c6cc(C(C)(C)C)cc7c8c9ccccc9cc(-c9ccc%10c(c9)c9ccccc9n%10-c9ccccc9)c8n(c5ccc4n2c31)c67 ; CC(C)(C)c1ccc2c(c1)c1cc3ccccc3c3c4c5c6cc(C(C)(C)C)cc7c8c9ccccc9cc(-c9ccc%10c(c9)c9ccccc9n%10-c9ccccc9)c8n(c5cnc4n2c13)c67 ; CC(C)(C)c1cc(N(c2ccccc2)c2ccccc2)c2c(c1)c1cc3ccccc3c3c4c5c6cc(C(C)(C)C)cc7c8c9ccccc9cc(N(c9ccccc9)c9ccccc9)c8n(c5cnc4n2c13)c67]

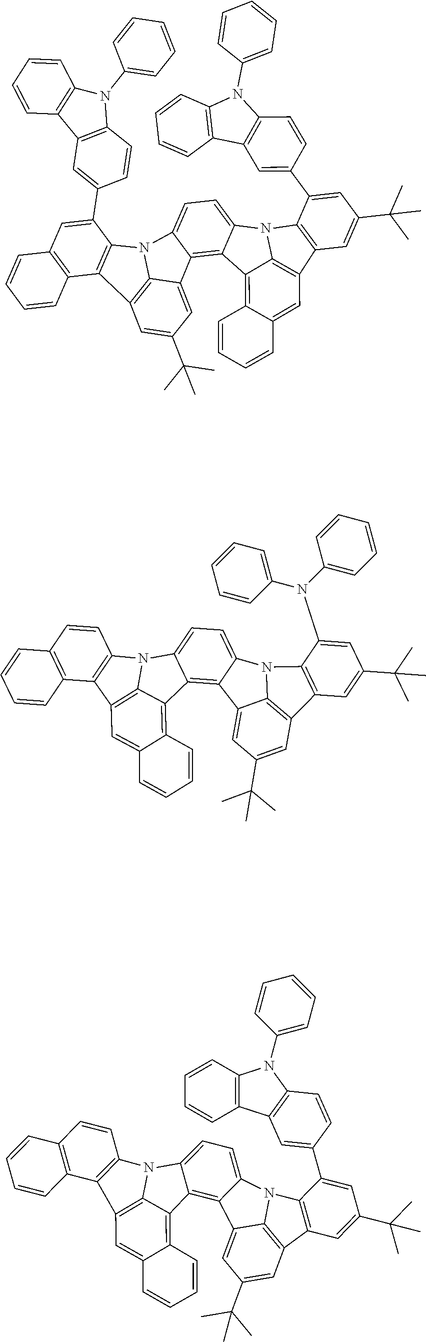 [SMILES: CC(C)(C)c1cc(-c2ccc3c(c2)c2ccccc2n3-c2ccccc2)c2c(c1)c1cc3ccccc3c3c4c5c6cc(C(C)(C)C)cc7c8c9ccccc9cc(-c9ccc%10c(c9)c9ccccc9n%10-c9ccccc9)c8n(c5ccc4n2c13)c67 ; CC(C)(C)c1cc(N(c2ccccc2)c2ccccc2)c2c(c1)c1cc(C(C)(C)C)cc3c4c5c6c7ccccc7cc7c8c9ccccc9ccc8n(c5ccc4n2c13)c76 ; CC(C)(C)c1cc(-c2ccc3c(c2)c2ccccc2n3-c2ccccc2)c2c(c1)c1cc(C(C)(C)C)cc3c4c5c6c7ccccc7cc7c8c9ccccc9ccc8n(c5ccc4n2c13)c76]

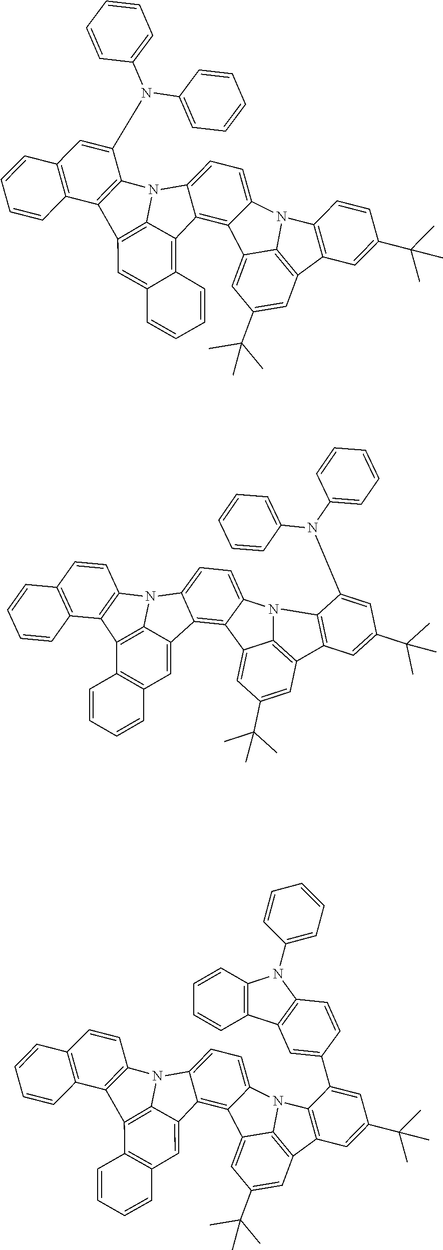 [SMILES: CC(C)(C)c1ccc2c(c1)c1cc(C(C)(C)C)cc3c4c5c6c7ccccc7cc7c8c9ccccc9cc(N(c9ccccc9)c9ccccc9)c8n(c5ccc4n2c13)c76 ; CC(C)(C)c1cc(N(c2ccccc2)c2ccccc2)c2c(c1)c1cc(C(C)(C)C)cc3c4c5c6cc7ccccc7c7c8c9ccccc9ccc8n(c5ccc4n2c13)c67 ; CC(C)(C)c1cc(-c2ccc3c(c2)c2ccccc2n3-c2ccccc2)c2c(c1)c1cc(C(C)(C)C)cc3c4c5c6cc7ccccc7c7c8c9ccccc9ccc8n(c5ccc4n2c13)c67]

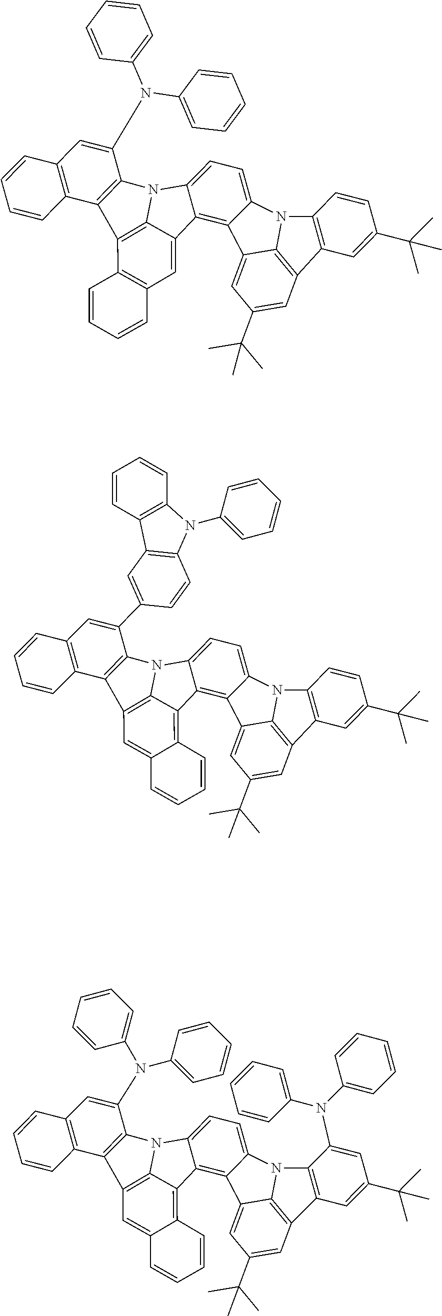 [SMILES: CC(C)(C)c1ccc2c(c1)c1cc(C(C)(C)C)cc3c4c5c6cc7ccccc7c7c8c9ccccc9cc(N(c9ccccc9)c9ccccc9)c8n(c5ccc4n2c13)c67 ; CC(C)(C)c1ccc2c(c1)c1cc(C(C)(C)C)cc3c4c5c6c7ccccc7cc7c8c9ccccc9cc(-c9ccc%10c(c9)c9ccccc9n%10-c9ccccc9)c8n(c5ccc4n2c13)c76 ; CC(C)(C)c1cc(N(c2ccccc2)c2ccccc2)c2c(c1)c1cc(C(C)(C)C)cc3c4c5c6c7ccccc7cc7c8c9ccccc9cc(N(c9ccccc9)c9ccccc9)c8n(c5ccc4n2c13)c76]

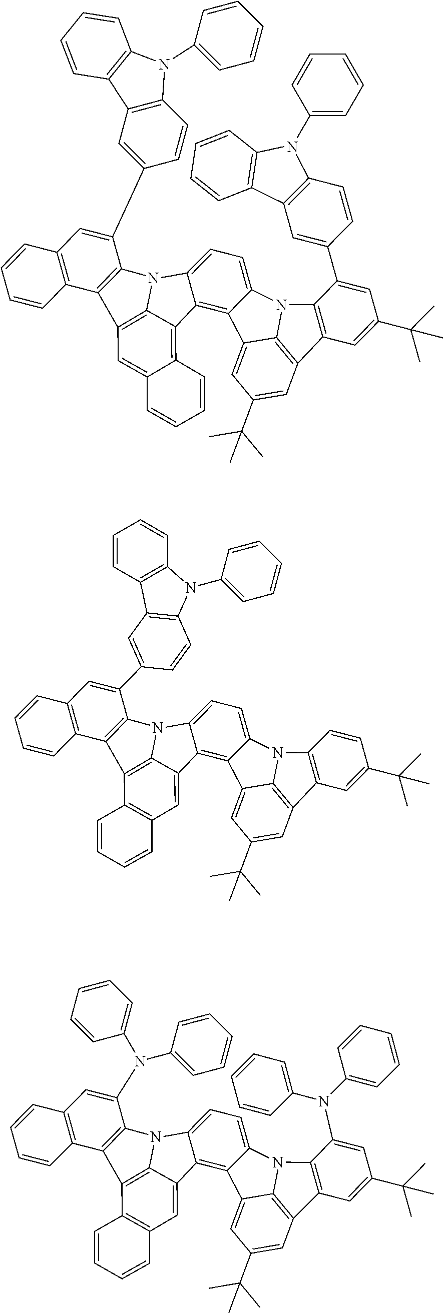 [SMILES: CC(C)(C)c1cc(-c2ccc3c(c2)c2ccccc2n3-c2ccccc2)c2c(c1)c1cc(C(C)(C)C)cc3c4c5c6c7ccccc7cc7c8c9ccccc9cc(-c9ccc%10c(c9)c9ccccc9n%10-c9ccccc9)c8n(c5ccc4n2c13)c76 ; CC(C)(C)c1ccc2c(c1)c1cc(C(C)(C)C)cc3c4c5c6cc7ccccc7c7c8c9ccccc9cc(-c9ccc%10c(c9)c9ccccc9n%10-c9ccccc9)c8n(c5ccc4n2c13)c67 ; CC(C)(C)c1cc(N(c2ccccc2)c2ccccc2)c2c(c1)c1cc(C(C)(C)C)cc3c4c5c6cc7ccccc7c7c8c9ccccc9cc(N(c9ccccc9)c9ccccc9)c8n(c5ccc4n2c13)c67]

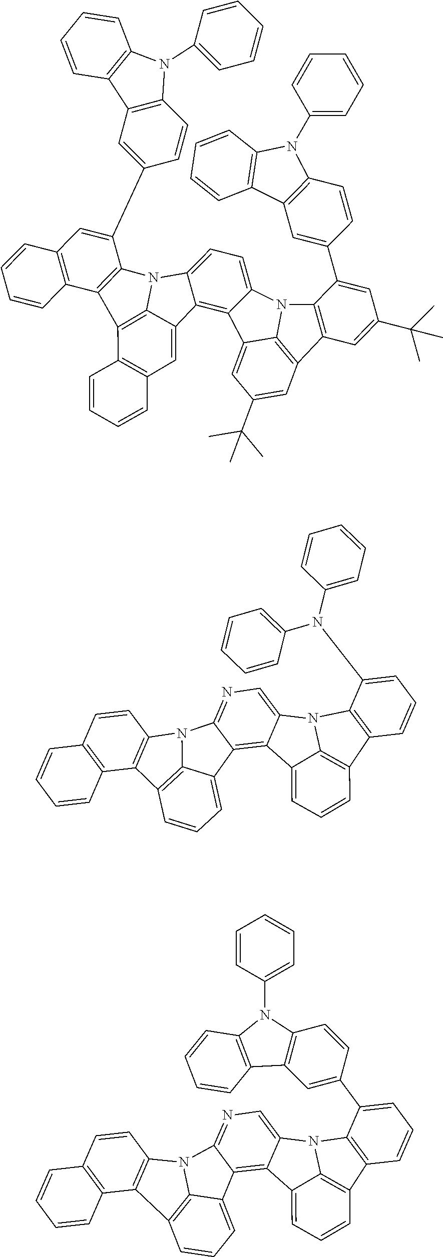 [SMILES: CC(C)(C)c1cc(-c2ccc3c(c2)c2ccccc2n3-c2ccccc2)c2c(c1)c1cc(C(C)(C)C)cc3c4c5c6cc7ccccc7c7c8c9ccccc9cc(-c9ccc%10c(c9)c9ccccc9n%10-c9ccccc9)c8n(c5ccc4n2c13)c67 ; c1ccc(N(c2ccccc2)c2cccc3c4cccc5c6c7c8cccc9c%10c%11ccccc%11ccc%10n(c7ncc6n(c23)c45)c98)cc1 ; c1ccc(-n2c3ccccc3c3cc(-c4cccc5c6cccc7c8c9c%10cccc%11c%12c%13ccccc%13ccc%12n(c9ncc8n(c45)c67)c%11%10)ccc32)cc1]

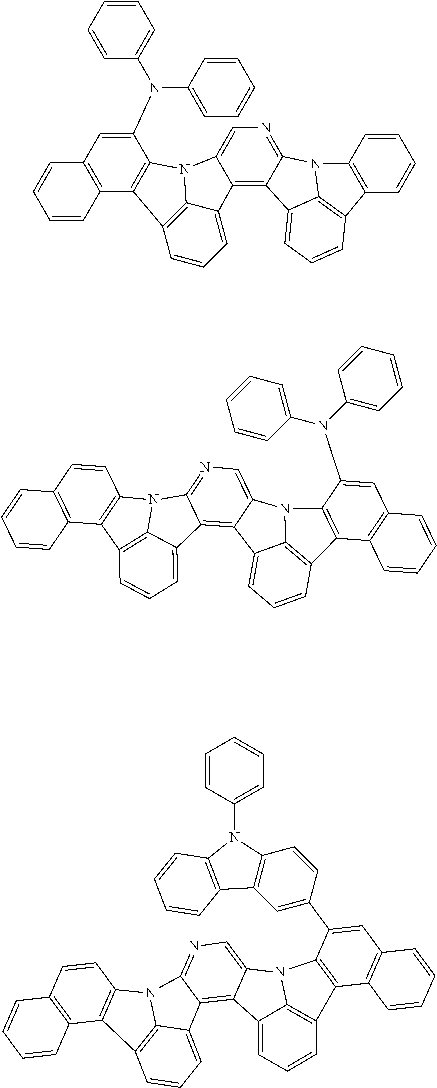 [SMILES: c1ccc(N(c2ccccc2)c2cc3ccccc3c3c4cccc5c6c7c8cccc9c%10ccccc%10n(c7ncc6n(c23)c54)c98)cc1 ; c1ccc(N(c2ccccc2)c2cc3ccccc3c3c4cccc5c6c7c8cccc9c%10c%11ccccc%11ccc%10n(c7ncc6n(c23)c54)c98)cc1 ; c1ccc(-n2c3ccccc3c3cc(-c4cc5ccccc5c5c6cccc7c8c9c%10cccc%11c%12c%13ccccc%13ccc%12n(c9ncc8n(c45)c76)c%11%10)ccc32)cc1]

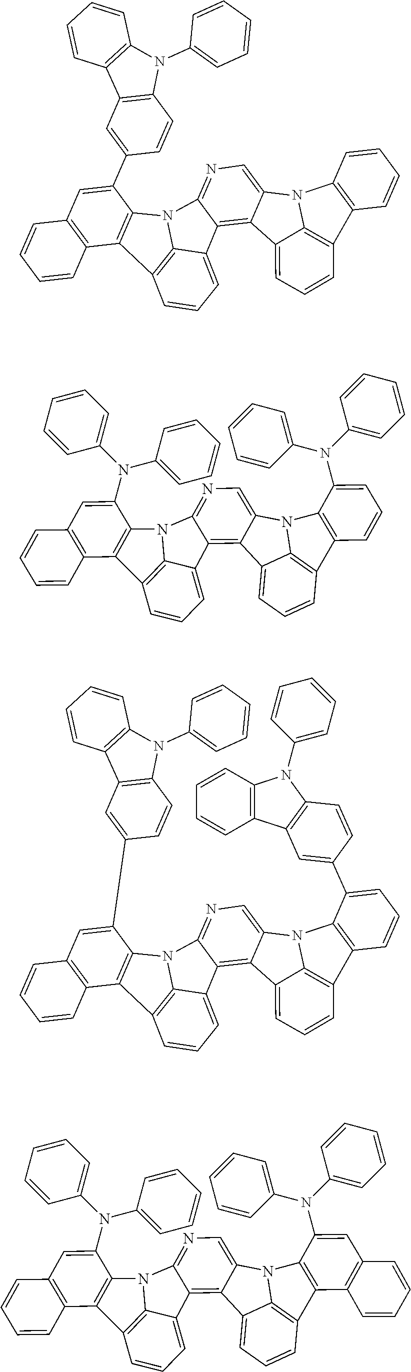

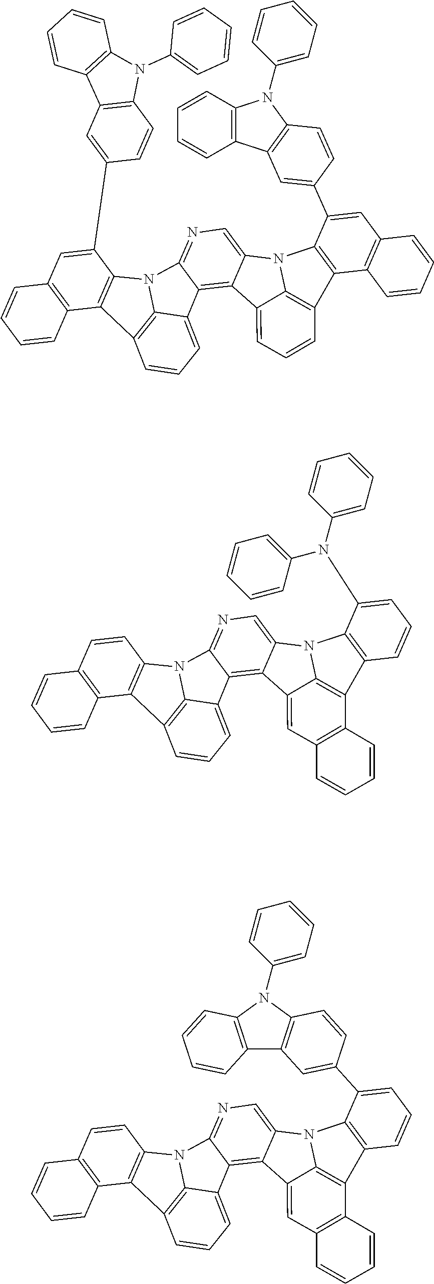 [SMILES: c1ccc(-n2c3ccccc3c3cc(-c4cc5ccccc5c5c6cccc7c8c9c%10cccc%11c%12c%13ccccc%13cc(-c%13ccc%14c(c%13)c%13ccccc%13n%14-c%13ccccc%13)c%12n(c9ncc8n(c45)c76)c%10%11)ccc32)cc1 ; c1ccc(N(c2ccccc2)c2cccc3c4c5ccccc5cc5c6c7c8cccc9c%10c%11ccccc%11ccc%10n(c7ncc6n(c23)c54)c98)cc1 ; c1ccc(-n2c3ccccc3c3cc(-c4cccc5c6c7ccccc7cc7c8c9c%10cccc%11c%12c%13ccccc%13ccc%12n(c9ncc8n(c45)c76)c%11%10)ccc32)cc1]

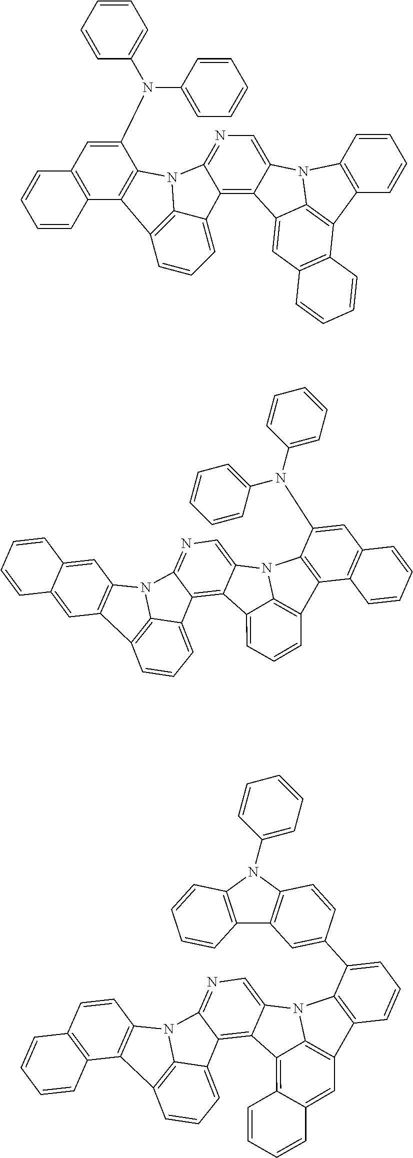 [SMILES: c1ccc(N(c2ccccc2)c2cc3ccccc3c3c4cccc5c6c7c8cc9ccccc9c9c%10ccccc%10n(c7cnc6n(c23)c54)c89)cc1 ; c1ccc(N(c2ccccc2)c2cc3ccccc3c3c4cccc5c6c7c8cccc9c%10cc%11ccccc%11cc%10n(c7ncc6n(c23)c54)c98)cc1 ; c1ccc(-n2c3ccccc3c3cc(-c4cccc5c6cc7ccccc7c7c8c9c%10cccc%11c%12c%13ccccc%13ccc%12n(c9ncc8n(c45)c67)c%11%10)ccc32)cc1]

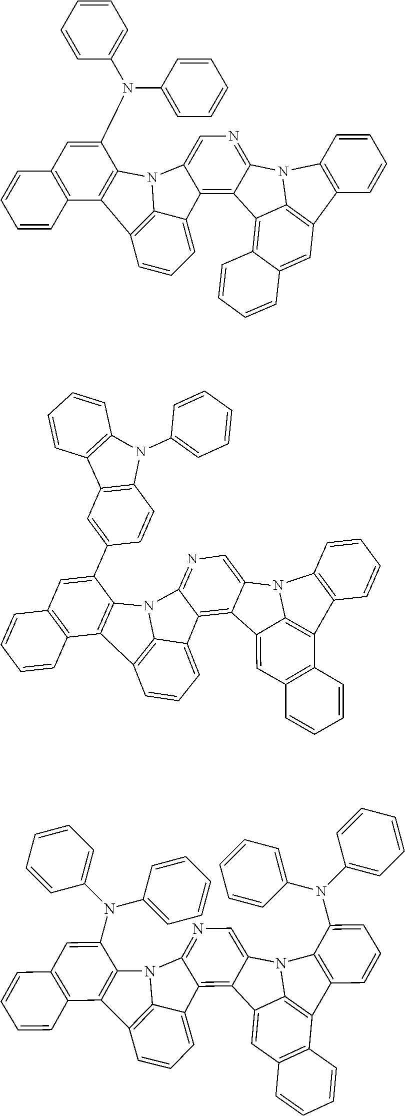 [SMILES: c1ccc(N(c2ccccc2)c2cc3ccccc3c3c4cccc5c6c7c8c9ccccc9cc9c%10ccccc%10n(c7ncc6n(c23)c54)c98)cc1 ; c1ccc(-n2c3ccccc3c3cc(-c4cc5ccccc5c5c6cccc7c8c9c%10cc%11ccccc%11c%11c%12ccccc%12n(c9cnc8n(c45)c76)c%10%11)ccc32)cc1 ; c1ccc(N(c2ccccc2)c2cccc3c4c5ccccc5cc5c6c7c8cccc9c%10c%11ccccc%11cc(N(c%11ccccc%11)c%11ccccc%11)c%10n(c7ncc6n(c23)c54)c89)cc1]

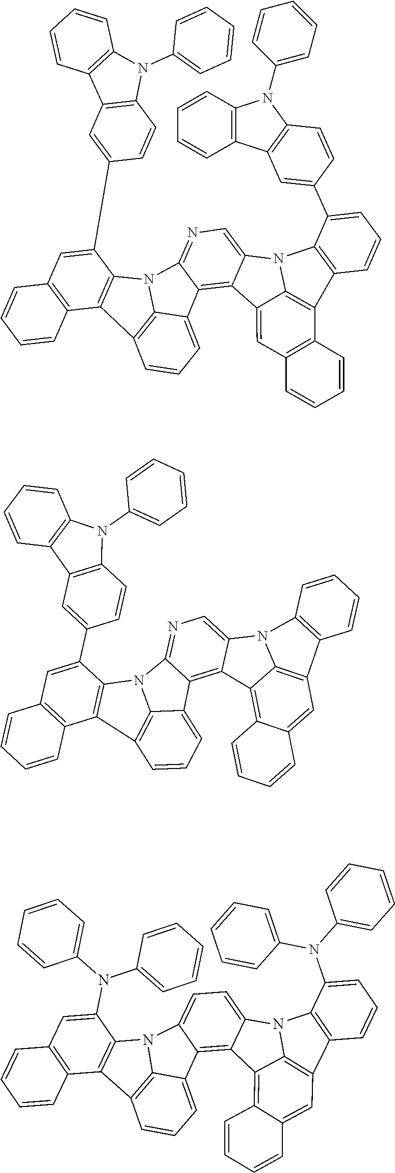 [SMILES: c1ccc(-n2c3ccccc3c3cc(-c4cccc5c6c7ccccc7cc7c8c9c%10cccc%11c%12c%13ccccc%13cc(-c%13ccc%14c(c%13)c%13ccccc%13n%14-c%13ccccc%13)c%12n(c9ncc8n(c45)c76)c%10%11)ccc32)cc1 ; c1ccc(-n2c3ccccc3c3cc(-c4cc5ccccc5c5c6cccc7c8c9c%10c%11ccccc%11cc%11c%12ccccc%12n(c9cnc8n(c45)c76)c%11%10)ccc32)cc1 ; c1ccc(N(c2ccccc2)c2cc3ccccc3c3c4cccc5c6c7c8c9ccccc9cc9c%10cccc(N(c%11ccccc%11)c%11ccccc%11)c%10n(c7ccc6n(c23)c54)c98)cc1]

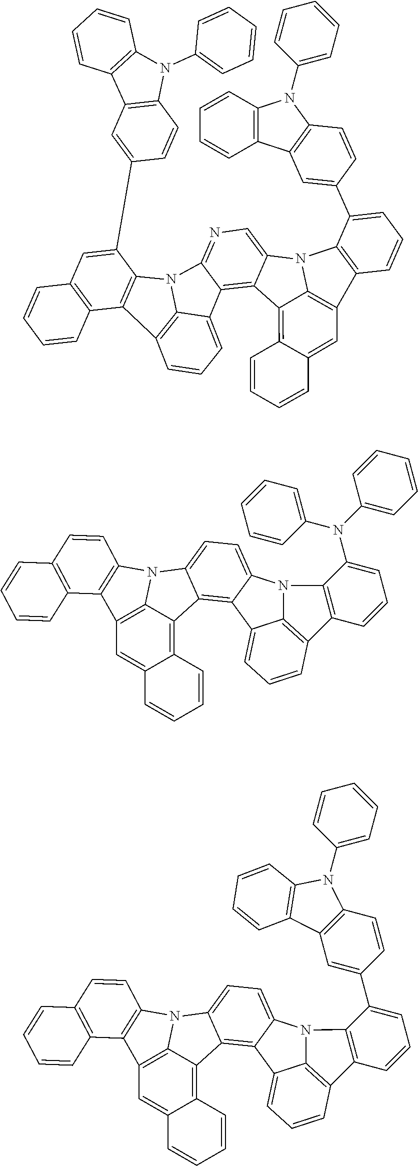 [SMILES: c1ccc(-n2c3ccccc3c3cc(-c4cccc5c6cc7ccccc7c7c8c9c%10cccc%11c%12c%13ccccc%13cc(-c%13ccc%14c(c%13)c%13ccccc%13n%14-c%13ccccc%13)c%12n(c9ncc8n(c45)c67)c%10%11)ccc32)cc1 ; c1ccc(N(c2ccccc2)c2cccc3c4cccc5c6c7c8c9ccccc9cc9c%10c%11ccccc%11ccc%10n(c7ccc6n(c23)c45)c98)cc1 ; c1ccc(-n2c3ccccc3c3cc(-c4cccc5c6cccc7c8c9c%10c%11ccccc%11cc%11c%12c%13ccccc%13ccc%12n(c9ccc8n(c45)c67)c%11%10)ccc32)cc1]

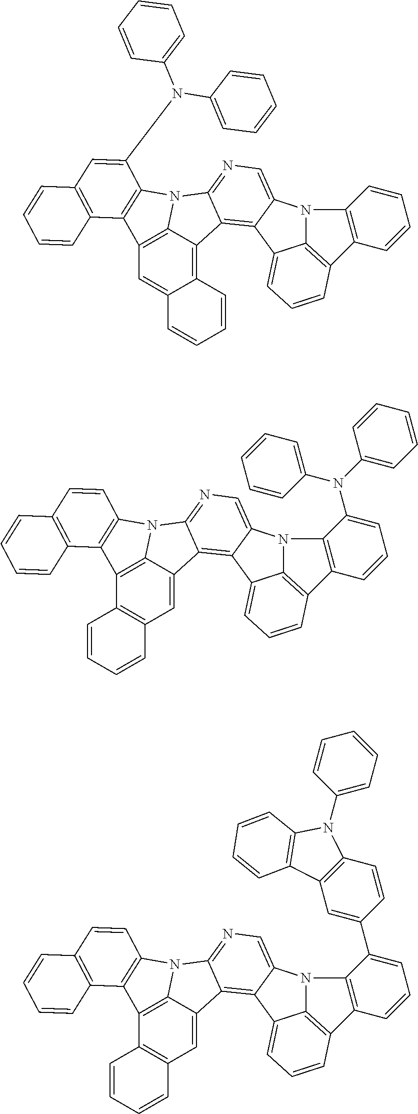 [SMILES: c1ccc(N(c2ccccc2)c2cc3ccccc3c3c4cc5ccccc5c5c6c7c8cccc9c%10ccccc%10n(c7cnc6n(c23)c45)c98)cc1 ; c1ccc(N(c2ccccc2)c2cccc3c4cccc5c6c7c8cc9ccccc9c9c%10c%11ccccc%11ccc%10n(c7ncc6n(c23)c45)c89)cc1 ; c1ccc(-n2c3ccccc3c3cc(-c4cccc5c6cccc7c8c9c%10cc%11ccccc%11c%11c%12c%13ccccc%13ccc%12n(c9ncc8n(c45)c67)c%10%11)ccc32)cc1]

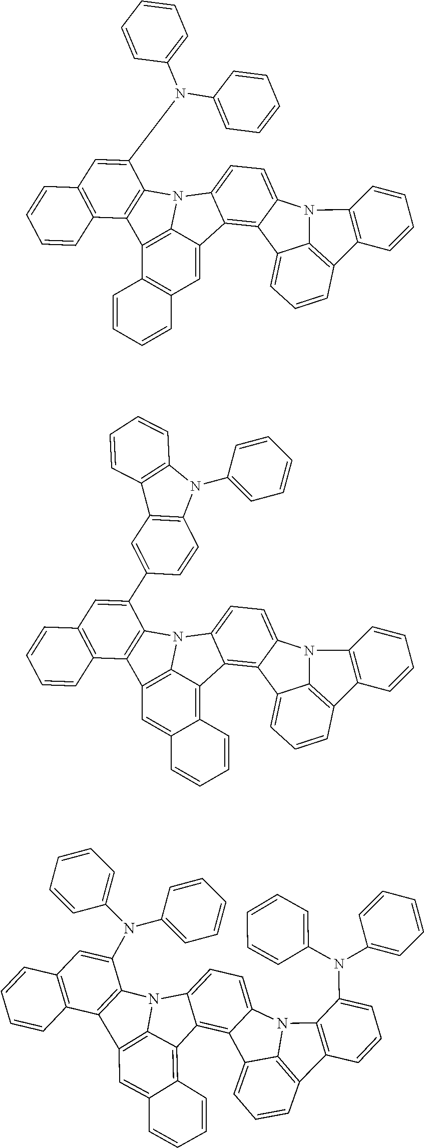 [SMILES: c1ccc(N(c2ccccc2)c2cc3ccccc3c3c4c5ccccc5cc5c6c7c8cccc9c%10ccccc%10n(c7ccc6n(c23)c54)c98)cc1 ; c1ccc(-n2c3ccccc3c3cc(-c4cc5ccccc5c5c6cc7ccccc7c7c8c9c%10cccc%11c%12ccccc%12n(c9ccc8n(c45)c67)c%11%10)ccc32)cc1 ; c1ccc(N(c2ccccc2)c2cccc3c4cccc5c6c7c8c9ccccc9cc9c%10c%11ccccc%11cc(N(c%11ccccc%11)c%11ccccc%11)c%10n(c7ccc6n(c23)c45)c98)cc1]

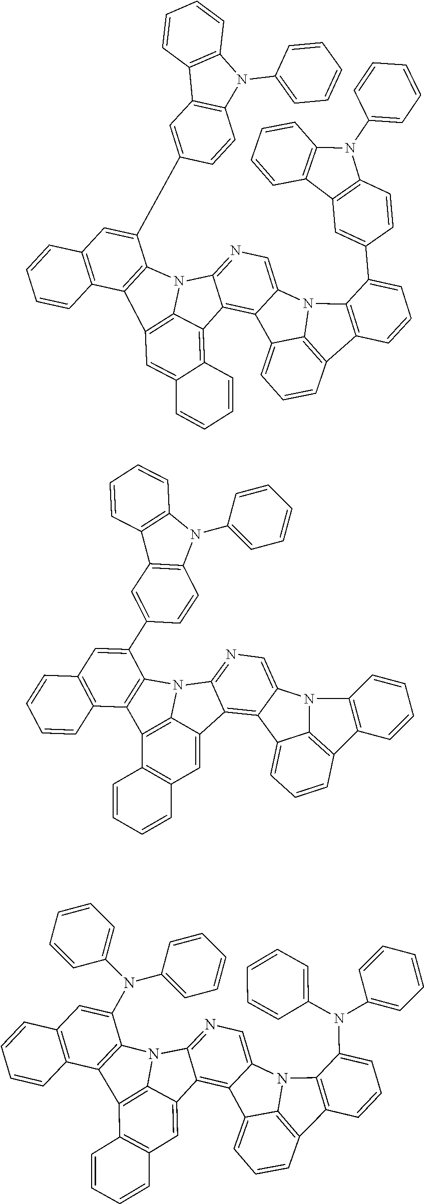 [SMILES: c1ccc(-n2c3ccccc3c3cc(-c4cccc5c6cccc7c8c9c%10c%11ccccc%11cc%11c%12c%13ccccc%13cc(-c%13ccc%14c(c%13)c%13ccccc%13n%14-c%13ccccc%13)c%12n(c9ncc8n(c45)c67)c%11%10)ccc32)cc1 ; c1ccc(-n2c3ccccc3c3cc(-c4cc5ccccc5c5c6c7ccccc7cc7c8c9c%10cccc%11c%12ccccc%12n(c9cnc8n(c45)c76)c%11%10)ccc32)cc1 ; c1ccc(N(c2ccccc2)c2cccc3c4cccc5c6c7c8cc9ccccc9c9c%10c%11ccccc%11cc(N(c%11ccccc%11)c%11ccccc%11)c%10n(c7ncc6n(c23)c45)c89)cc1]

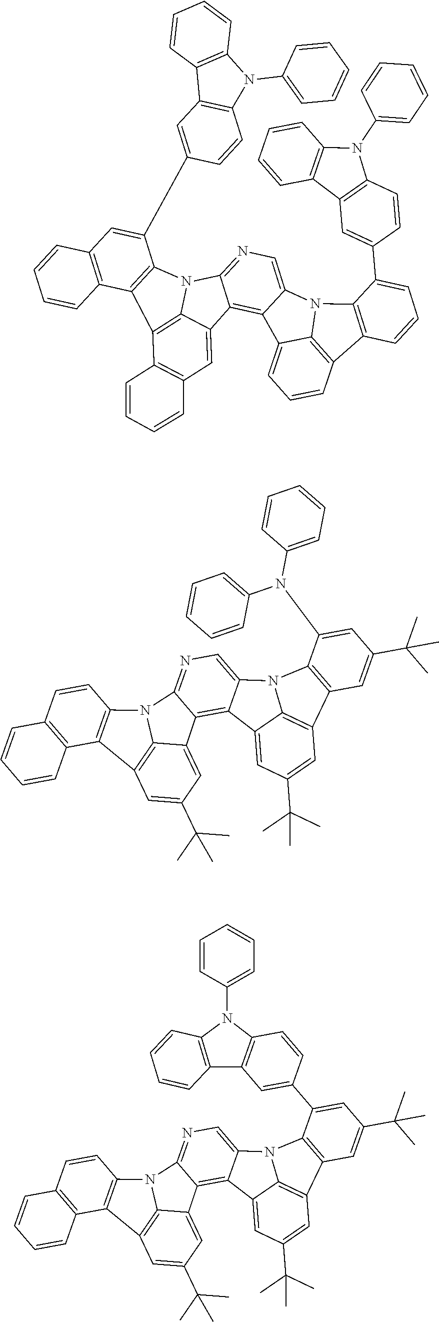 [SMILES: c1ccc(-n2c3ccccc3c3cc(-c4cccc5c6cccc7c8c9c%10cc%11ccccc%11c%11c%12c%13ccccc%13cc(-c%13ccc%14c(c%13)c%13ccccc%13n%14-c%13ccccc%13)c%12n(c9ncc8n(c45)c67)c%10%11)ccc32)cc1 ; CC(C)(C)c1cc(N(c2ccccc2)c2ccccc2)c2c(c1)c1cc(C(C)(C)C)cc3c4c5c6cc(C(C)(C)C)cc7c8c9ccccc9ccc8n(c5ncc4n2c13)c76 ; CC(C)(C)c1cc(-c2ccc3c(c2)c2ccccc2n3-c2ccccc2)c2c(c1)c1cc(C(C)(C)C)cc3c4c5c6cc(C(C)(C)C)cc7c8c9ccccc9ccc8n(c5ncc4n2c13)c76]

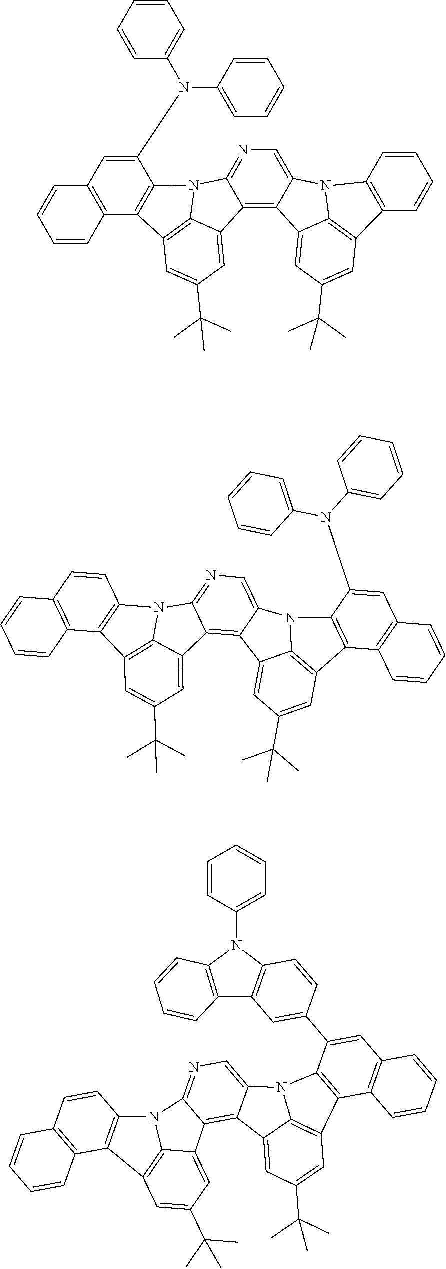 [SMILES: CC(C)(C)c1cc2c3ccccc3n3c4cnc5c(c6cc(C(C)(C)C)cc7c8c9ccccc9cc(N(c9ccccc9)c9ccccc9)c8n5c67)c4c(c1)c23 ; CC(C)(C)c1cc2c3c4ccccc4ccc3n3c4ncc5c(c6cc(C(C)(C)C)cc7c8c9ccccc9cc(N(c9ccccc9)c9ccccc9)c8n5c67)c4c(c1)c23 ; CC(C)(C)c1cc2c3c4ccccc4ccc3n3c4ncc5c(c6cc(C(C)(C)C)cc7c8c9ccccc9cc(-c9ccc%10c(c9)c9ccccc9n%10-c9ccccc9)c8n5c67)c4c(c1)c23]

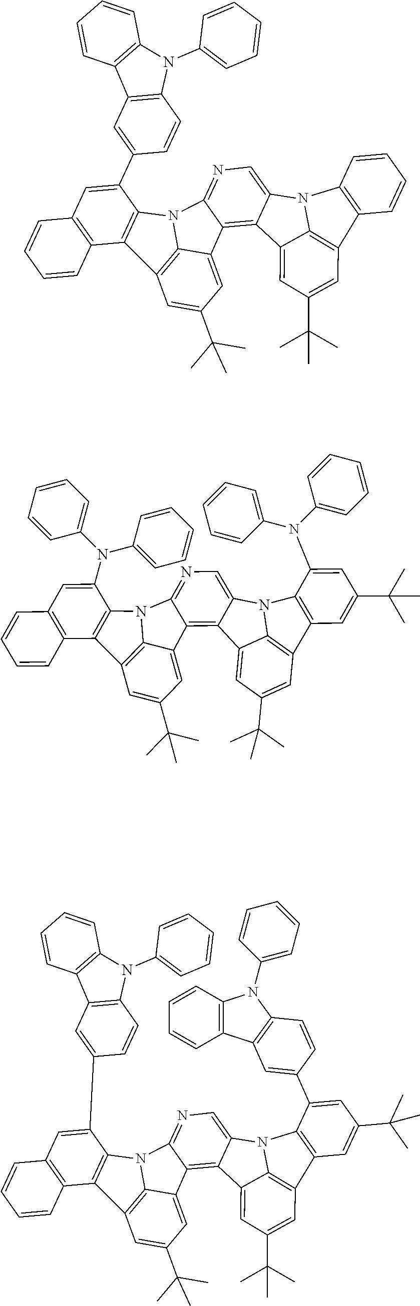 [SMILES: CC(C)(C)c1cc2c3ccccc3n3c4cnc5c(c6cc(C(C)(C)C)cc7c8c9ccccc9cc(-c9ccc%10c(c9)c9ccccc9n%10-c9ccccc9)c8n5c67)c4c(c1)c23 ; CC(C)(C)c1cc(N(c2ccccc2)c2ccccc2)c2c(c1)c1cc(C(C)(C)C)cc3c4c5c6cc(C(C)(C)C)cc7c8c9ccccc9cc(N(c9ccccc9)c9ccccc9)c8n(c5ncc4n2c13)c67 ; CC(C)(C)c1cc(-c2ccc3c(c2)c2ccccc2n3-c2ccccc2)c2c(c1)c1cc(C(C)(C)C)cc3c4c5c6cc(C(C)(C)C)cc7c8c9ccccc9cc(-c9ccc%10c(c9)c9ccccc9n%10-c9ccccc9)c8n(c5ncc4n2c13)c67]

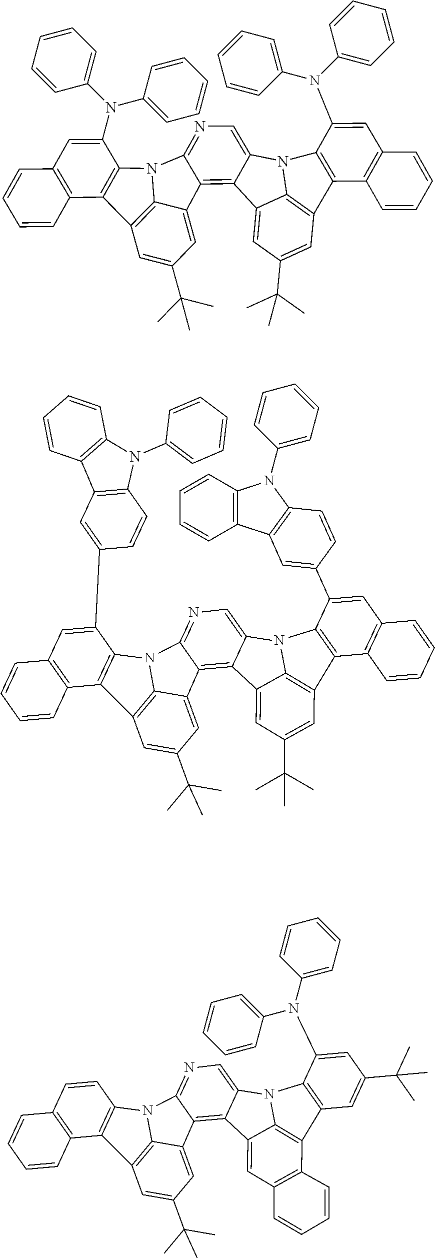 [SMILES: CC(C)(C)c1cc2c3c4c5cc(C(C)(C)C)cc6c7c8ccccc8cc(N(c8ccccc8)c8ccccc8)c7n(c4ncc3n3c4c(N(c7ccccc7)c7ccccc7)cc7ccccc7c4c(c1)c23)c56 ; CC(C)(C)c1cc2c3c4c5cc(C(C)(C)C)cc6c7c8ccccc8cc(-c8ccc9c(c8)c8ccccc8n9-c8ccccc8)c7n(c4ncc3n3c4c(-c7ccc8c(c7)c7ccccc7n8-c7ccccc7)cc7ccccc7c4c(c1)c23)c56 ; CC(C)(C)c1cc(N(c2ccccc2)c2ccccc2)c2c(c1)c1c3ccccc3cc3c4c5c6cc(C(C)(C)C)cc7c8c9ccccc9ccc8n(c5ncc4n2c31)c76]

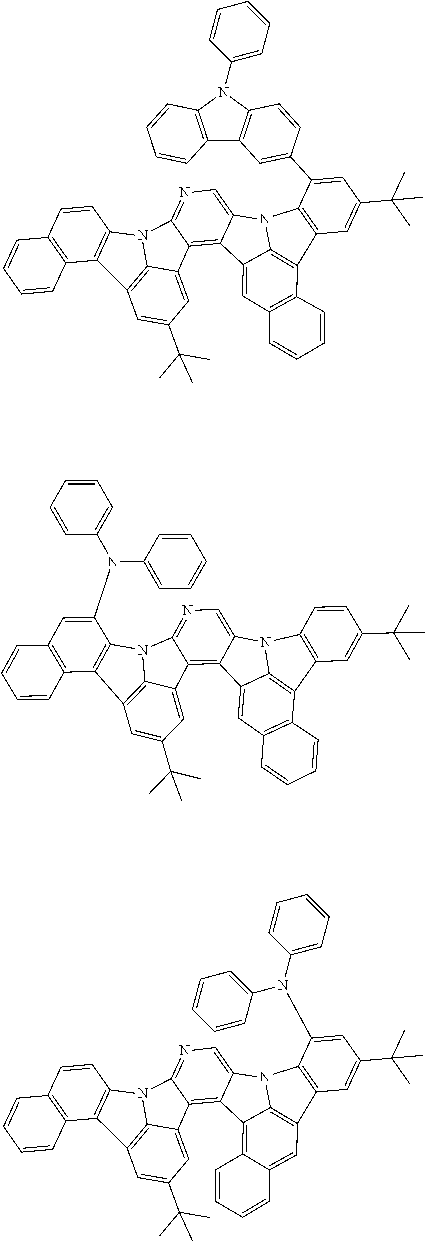 [SMILES: CC(C)(C)c1cc(-c2ccc3c(c2)c2ccccc2n3-c2ccccc2)c2c(c1)c1c3ccccc3cc3c4c5c6cc(C(C)(C)C)cc7c8c9ccccc9ccc8n(c5ncc4n2c31)c76 ; CC(C)(C)c1ccc2c(c1)c1c3ccccc3cc3c4c5c6cc(C(C)(C)C)cc7c8c9ccccc9cc(N(c9ccccc9)c9ccccc9)c8n(c5ncc4n2c31)c67 ; CC(C)(C)c1cc(N(c2ccccc2)c2ccccc2)c2c(c1)c1cc3ccccc3c3c4c5c6cc(C(C)(C)C)cc7c8c9ccccc9ccc8n(c5ncc4n2c13)c76]

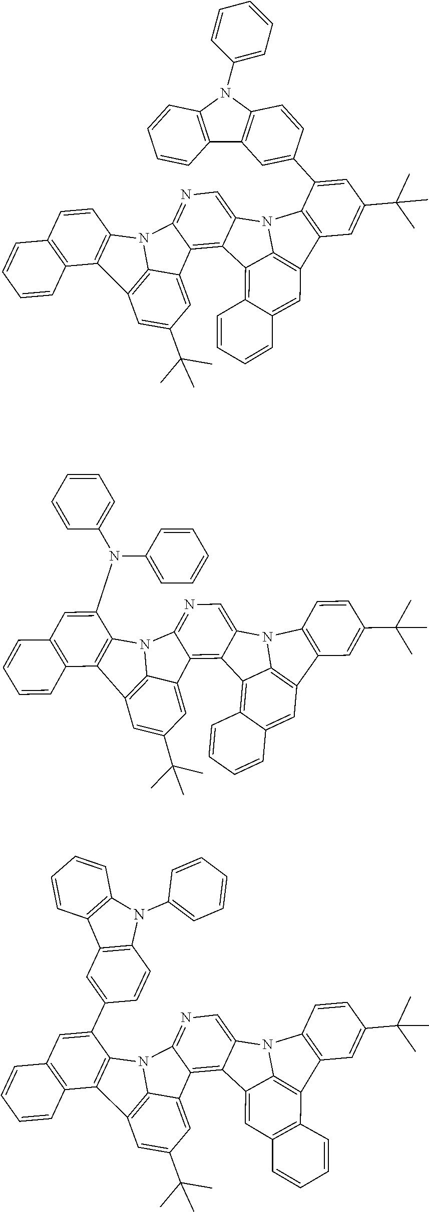 [SMILES: CC(C)(C)c1cc(-c2ccc3c(c2)c2ccccc2n3-c2ccccc2)c2c(c1)c1cc3ccccc3c3c4c5c6cc(C(C)(C)C)cc7c8c9ccccc9ccc8n(c5ncc4n2c13)c76 ; CC(C)(C)c1ccc2c(c1)c1cc3ccccc3c3c4c5c6cc(C(C)(C)C)cc7c8c9ccccc9cc(N(c9ccccc9)c9ccccc9)c8n(c5ncc4n2c13)c67 ; CC(C)(C)c1ccc2c(c1)c1c3ccccc3cc3c4c5c6cc(C(C)(C)C)cc7c8c9ccccc9cc(-c9ccc%10c(c9)c9ccccc9n%10-c9ccccc9)c8n(c5ncc4n2c31)c67]

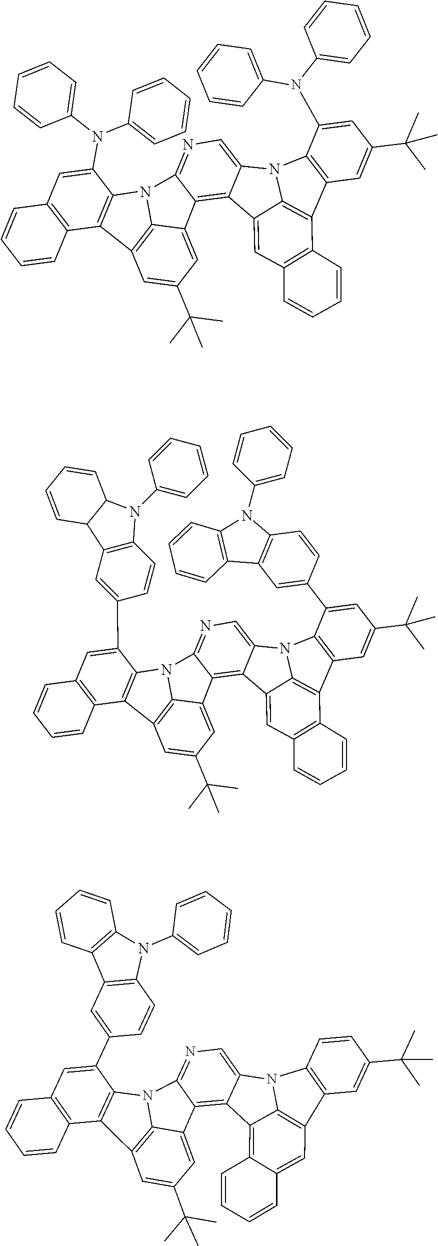 [SMILES: CC(C)(C)c1cc(N(c2ccccc2)c2ccccc2)c2c(c1)c1c3ccccc3cc3c4c5c6cc(C(C)(C)C)cc7c8c9ccccc9cc(N(c9ccccc9)c9ccccc9)c8n(c5ncc4n2c31)c67 ; CC(C)(C)c1cc(-c2ccc3c(c2)c2ccccc2n3-c2ccccc2)c2c(c1)c1c3ccccc3cc3c4c5c6cc(C(C)(C)C)cc7c8c9ccccc9cc(-c9ccc%10c(c9)C9C=CC=CC9N%10c9ccccc9)c8n(c5ncc4n2c31)c67 ; CC(C)(C)c1ccc2c(c1)c1cc3ccccc3c3c4c5c6cc(C(C)(C)C)cc7c8c9ccccc9cc(-c9ccc%10c(c9)c9ccccc9n%10-c9ccccc9)c8n(c5ncc4n2c13)c67]

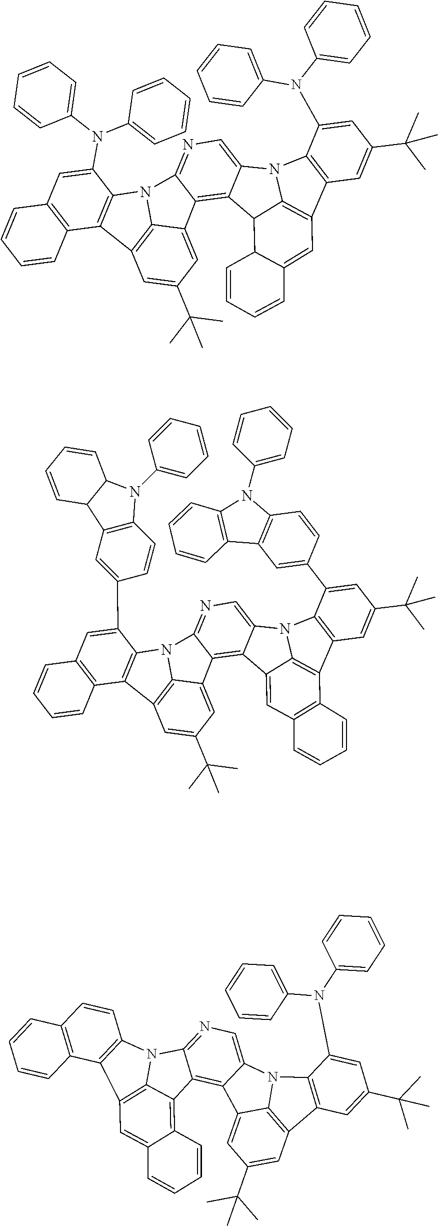 [SMILES: CC(C)(C)c1cc(N(c2ccccc2)c2ccccc2)c2c(c1)c1c3n2-c2cnc4c(c2C3C2C=CC=CC2=C1)c1cc(C(C)(C)C)cc2c3c5ccccc5cc(N(c5ccccc5)c5ccccc5)c3n4c12 ; CC(C)(C)c1cc(-c2ccc3c(c2)c2ccccc2n3-c2ccccc2)c2c(c1)c1c3ccccc3cc3c4c5c6cc(C(C)(C)C)cc7c8c9ccccc9cc(-c9ccc%10c(c9)C9C=CC=CC9N%10c9ccccc9)c8n(c5ncc4n2c31)c67 ; CC(C)(C)c1cc(N(c2ccccc2)c2ccccc2)c2c(c1)c1cc(C(C)(C)C)cc3c4c5c6c7ccccc7cc7c8c9ccccc9ccc8n(c5ncc4n2c13)c76]

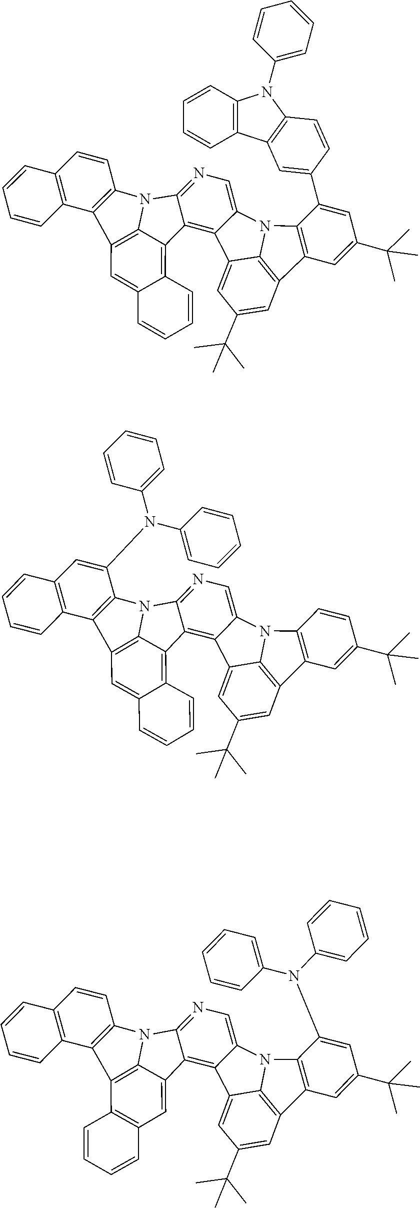 [SMILES: CC(C)(C)c1cc(-c2ccc3c(c2)c2ccccc2n3-c2ccccc2)c2c(c1)c1cc(C(C)(C)C)cc3c4c5c6c7ccccc7cc7c8c9ccccc9ccc8n(c5ncc4n2c13)c76 ; CC(C)(C)c1ccc2c(c1)c1cc(C(C)(C)C)cc3c4c5c6c7ccccc7cc7c8c9ccccc9cc(N(c9ccccc9)c9ccccc9)c8n(c5ncc4n2c13)c76 ; CC(C)(C)c1cc(N(c2ccccc2)c2ccccc2)c2c(c1)c1cc(C(C)(C)C)cc3c4c5c6cc7ccccc7c7c8c9ccccc9ccc8n(c5ncc4n2c13)c67]

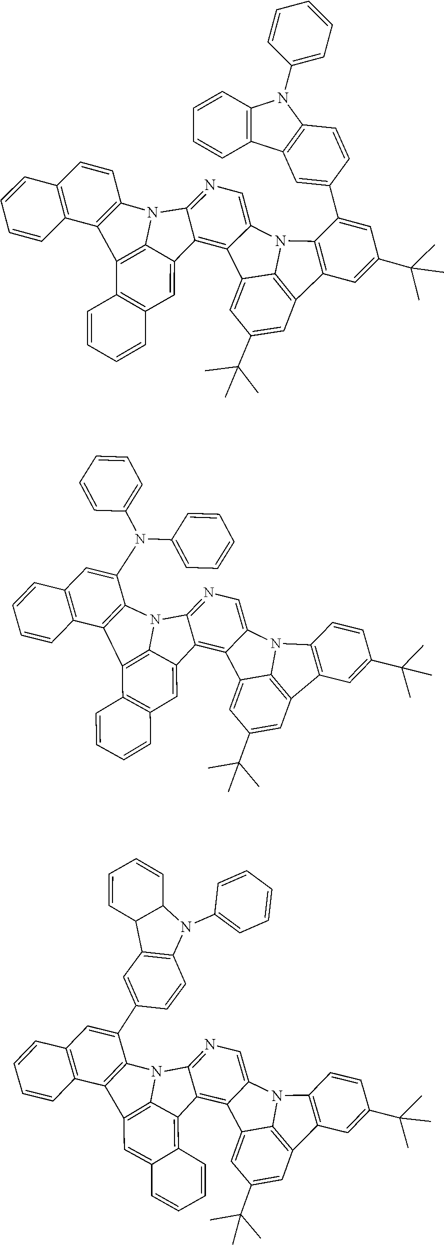 [SMILES: CC(C)(C)c1cc(-c2ccc3c(c2)c2ccccc2n3-c2ccccc2)c2c(c1)c1cc(C(C)(C)C)cc3c4c5c6cc7ccccc7c7c8c9ccccc9ccc8n(c5ncc4n2c13)c67 ; CC(C)(C)c1ccc2c(c1)c1cc(C(C)(C)C)cc3c4c5c6cc7ccccc7c7c8c9ccccc9cc(N(c9ccccc9)c9ccccc9)c8n(c5ncc4n2c13)c67 ; CC(C)(C)c1ccc2c(c1)c1cc(C(C)(C)C)cc3c4c5c6c7ccccc7cc7c8c9ccccc9cc(-c9ccc%10c(c9)C9C=CC=CC9N%10c9ccccc9)c8n(c5ncc4n2c13)c76]

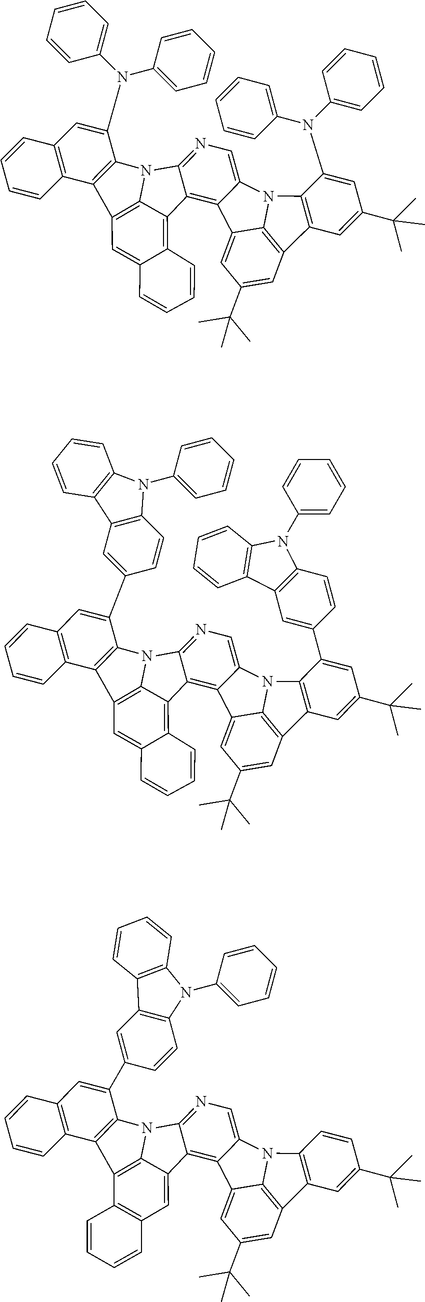 [SMILES: CC(C)(C)c1cc(N(c2ccccc2)c2ccccc2)c2c(c1)c1cc(C(C)(C)C)cc3c4c5c6c7ccccc7cc7c8c9ccccc9cc(N(c9ccccc9)c9ccccc9)c8n(c5ncc4n2c13)c76 ; CC(C)(C)c1cc(-c2ccc3c(c2)c2ccccc2n3-c2ccccc2)c2c(c1)c1cc(C(C)(C)C)cc3c4c5c6c7ccccc7cc7c8c9ccccc9cc(-c9ccc%10c(c9)c9ccccc9n%10-c9ccccc9)c8n(c5ncc4n2c13)c76 ; CC(C)(C)c1ccc2c(c1)c1cc(C(C)(C)C)cc3c4c5c6cc7ccccc7c7c8c9ccccc9cc(-c9ccc%10c(c9)c9ccccc9n%10-c9ccccc9)c8n(c5ncc4n2c13)c67]

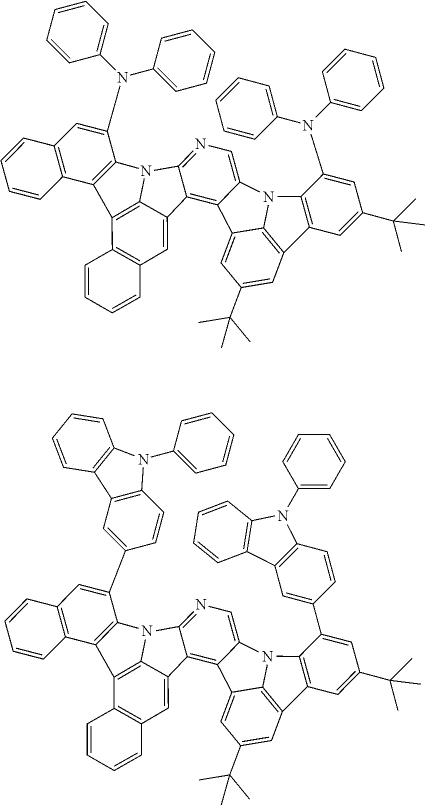 [SMILES: CC(C)(C)c1cc(N(c2ccccc2)c2ccccc2)c2c(c1)c1cc(C(C)(C)C)cc3c4c5c6cc7ccccc7c7c8c9ccccc9cc(N(c9ccccc9)c9ccccc9)c8n(c5ncc4n2c13)c67 ; CC(C)(C)c1cc(-c2ccc3c(c2)c2ccccc2n3-c2ccccc2)c2c(c1)c1cc(C(C)(C)C)cc3c4c5c6cc7ccccc7c7c8c9ccccc9cc(-c9ccc%10c(c9)c9ccccc9n%10-c9ccccc9)c8n(c5ncc4n2c13)c67]

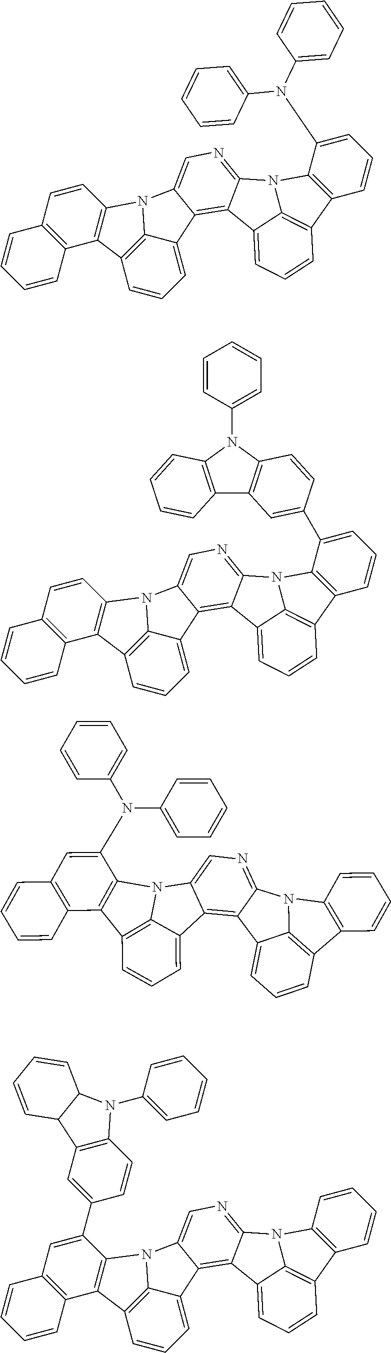

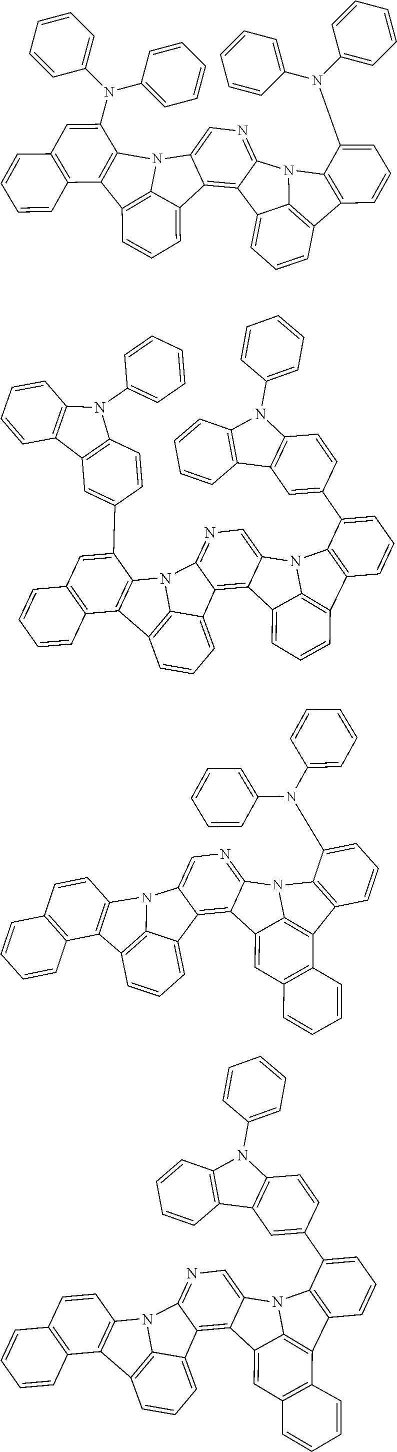

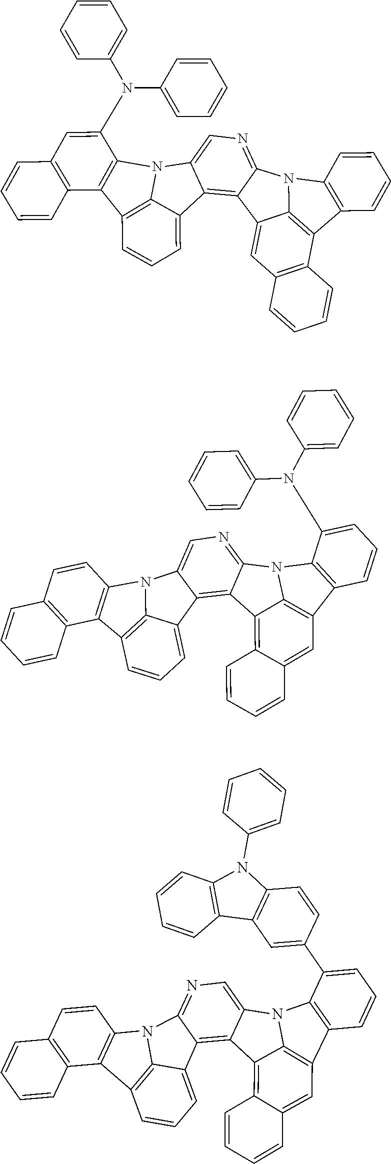 [SMILES: c1ccc(N(c2ccccc2)c2cc3ccccc3c3c4cccc5c6c7c8cc9ccccc9c9c%10ccccc%10n(c7ncc6n(c23)c54)c89)cc1 ; c1ccc(N(c2ccccc2)c2cccc3c4cc5ccccc5c5c6c7c8cccc9c%10c%11ccccc%11ccc%10n(c7cnc6n(c23)c45)c98)cc1 ; c1ccc(-n2c3ccccc3c3cc(-c4cccc5c6cc7ccccc7c7c8c9c%10cccc%11c%12c%13ccccc%13ccc%12n(c9ncc8n(c45)c67)c%11%10)ccc32)cc1]

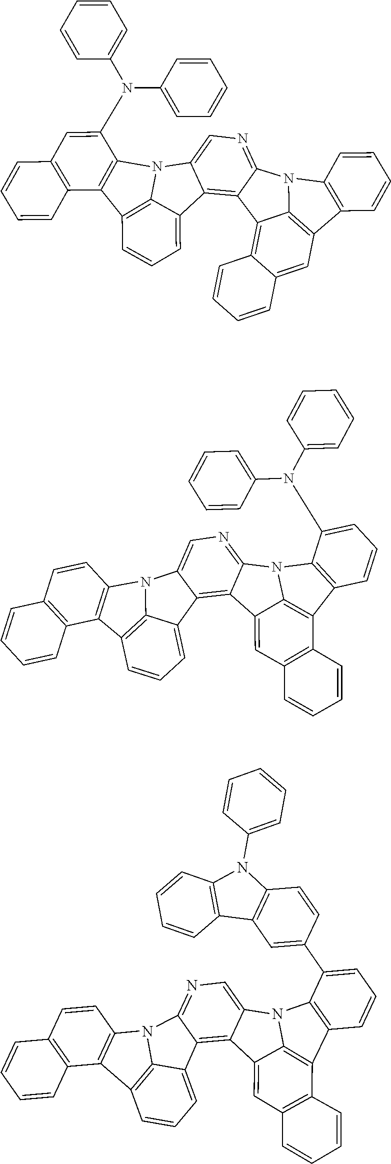 [SMILES: c1ccc(N(c2ccccc2)c2cc3ccccc3c3c4cccc5c6c7c8c9ccccc9cc9c%10ccccc%10n(c7ncc6n(c23)c54)c98)cc1 ; c1ccc(N(c2ccccc2)c2cccc3c4c5ccccc5cc5c6c7c8cccc9c%10c%11ccccc%11ccc%10n(c7cnc6n(c23)c54)c98)cc1 ; c1ccc(-n2c3ccccc3c3cc(-c4cccc5c6c7ccccc7cc7c8c9c%10cccc%11c%12c%13ccccc%13ccc%12n(c9ncc8n(c45)c76)c%11%10)ccc32)cc1]

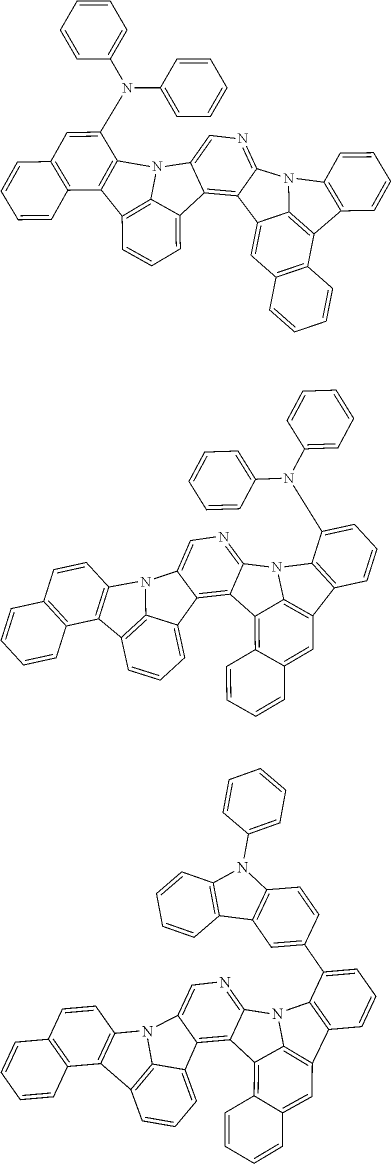 [SMILES: c1ccc(N(c2ccccc2)c2cc3ccccc3c3c4cccc5c6c7c8cc9ccccc9c9c%10ccccc%10n(c7ncc6n(c23)c54)c89)cc1 ; c1ccc(N(c2ccccc2)c2cccc3c4cc5ccccc5c5c6c7c8cccc9c%10c%11ccccc%11ccc%10n(c7cnc6n(c23)c45)c98)cc1 ; c1ccc(-n2c3ccccc3c3cc(-c4cccc5c6cc7ccccc7c7c8c9c%10cccc%11c%12c%13ccccc%13ccc%12n(c9cnc8n(c45)c67)c%11%10)ccc32)cc1]

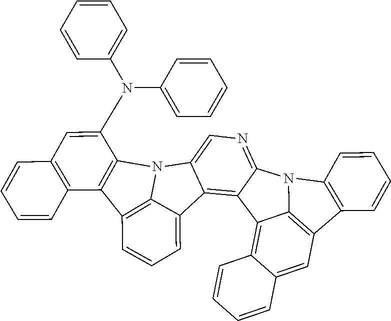 [SMILES: c1ccc(N(c2ccccc2)c2cc3ccccc3c3c4cccc5c6c7c8c9ccccc9cc9c%10ccccc%10n(c7ncc6n(c23)c54)c98)cc1]

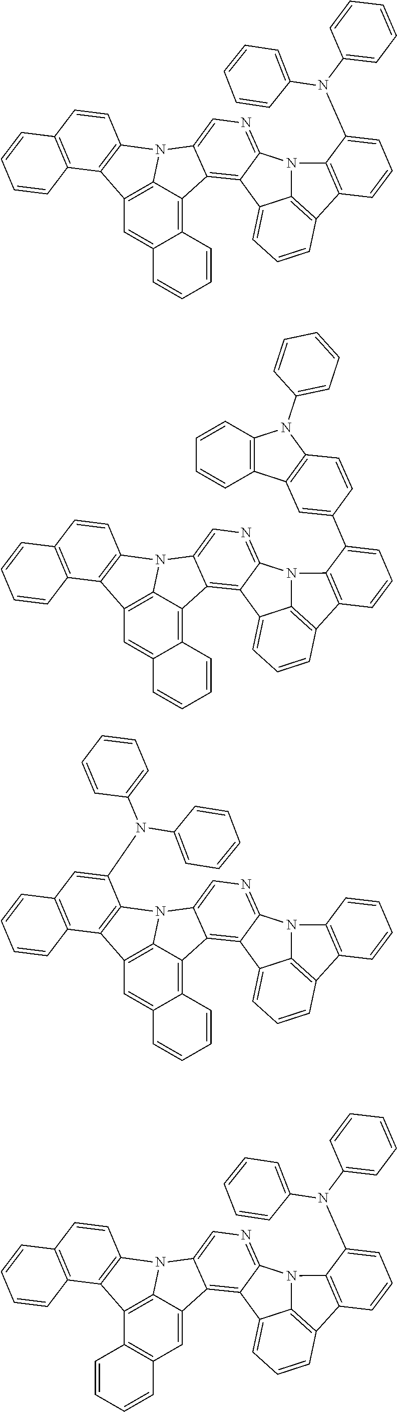 [SMILES: c1ccc(N(c2ccccc2)c2cccc3c4cccc5c6c7c8c9ccccc9cc9c%10c%11ccccc%11ccc%10n(c7cnc6n(c23)c45)c98)cc1 ; c1ccc(-n2c3ccccc3c3cc(-c4cccc5c6cccc7c8c9c%10c%11ccccc%11cc%11c%12c%13ccccc%13ccc%12n(c9cnc8n(c45)c67)c%11%10)ccc32)cc1 ; c1ccc(N(c2ccccc2)c2cc3ccccc3c3c4cc5ccccc5c5c6c7c8cccc9c%10ccccc%10n(c7ncc6n(c23)c45)c98)cc1 ; c1ccc(N(c2ccccc2)c2cccc3c4cccc5c6c7c8cc9ccccc9c9c%10c%11ccccc%11ccc%10n(c7cnc6n(c23)c45)c89)cc1]

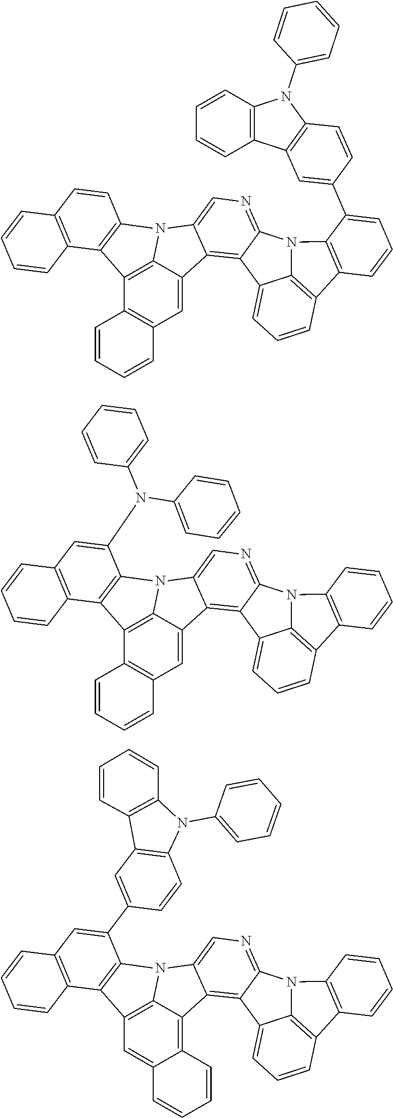

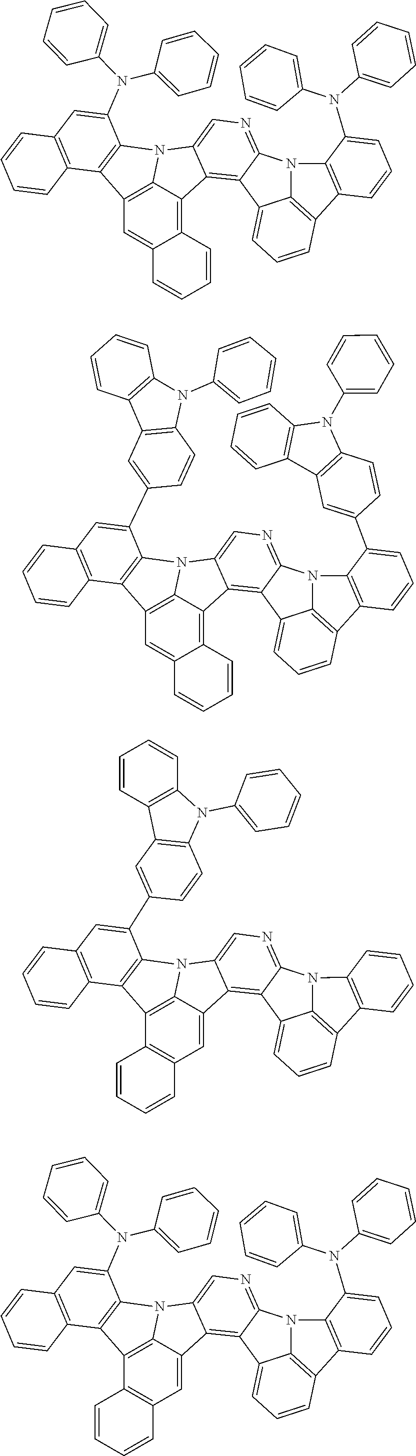

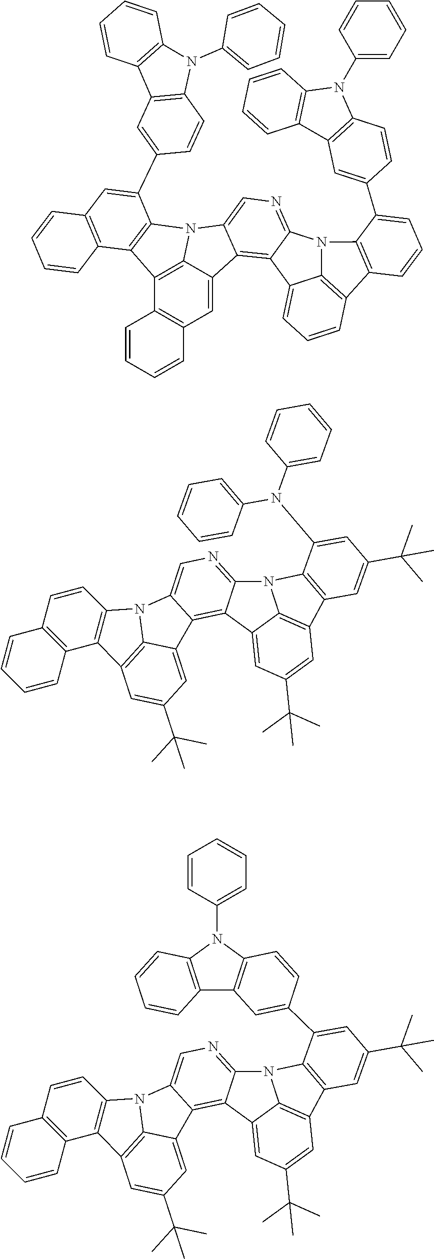 [SMILES: c1ccc(-n2c3ccccc3c3cc(-c4cc5ccccc5c5c6c7ccccc7cc7c8c9c%10cccc%11c%12cccc(-c%13ccc%14c(c%13)c%13ccccc%13n%14-c%13ccccc%13)c%12n(c9ncc8n(c45)c76)c%11%10)ccc32)cc1 ; CC(C)(C)c1cc(N(c2ccccc2)c2ccccc2)c2c(c1)c1cc(C(C)(C)C)cc3c4c5c6cc(C(C)(C)C)cc7c8c9ccccc9ccc8n(c5cnc4n2c13)c76 ; CC(C)(C)c1cc(-c2ccc3c(c2)c2ccccc2n3-c2ccccc2)c2c(c1)c1cc(C(C)(C)C)cc3c4c5c6cc(C(C)(C)C)cc7c8c9ccccc9ccc8n(c5cnc4n2c13)c76]

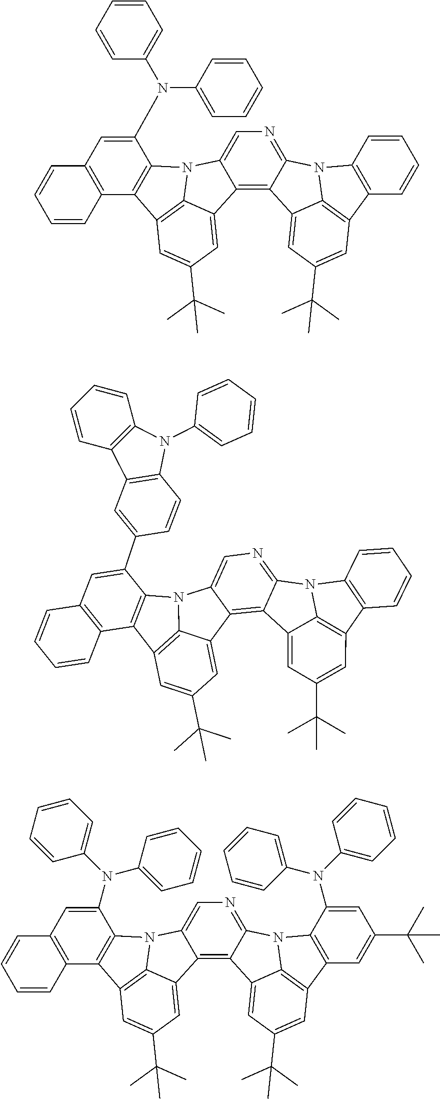 [SMILES: CC(C)(C)c1cc2c3ccccc3n3c4ncc5c(c6cc(C(C)(C)C)cc7c8c9ccccc9cc(N(c9ccccc9)c9ccccc9)c8n5c67)c4c(c1)c23 ; CC(C)(C)c1cc2c3ccccc3n3c4ncc5c(c6cc(C(C)(C)C)cc7c8c9ccccc9cc(-c9ccc%10c(c9)c9ccccc9n%10-c9ccccc9)c8n5c67)c4c(c1)c23 ; CC(C)(C)c1cc(N(c2ccccc2)c2ccccc2)c2c(c1)c1cc(C(C)(C)C)cc3c4c5c6cc(C(C)(C)C)cc7c8c9ccccc9cc(N(c9ccccc9)c9ccccc9)c8n(c5cnc4n2c13)c67]

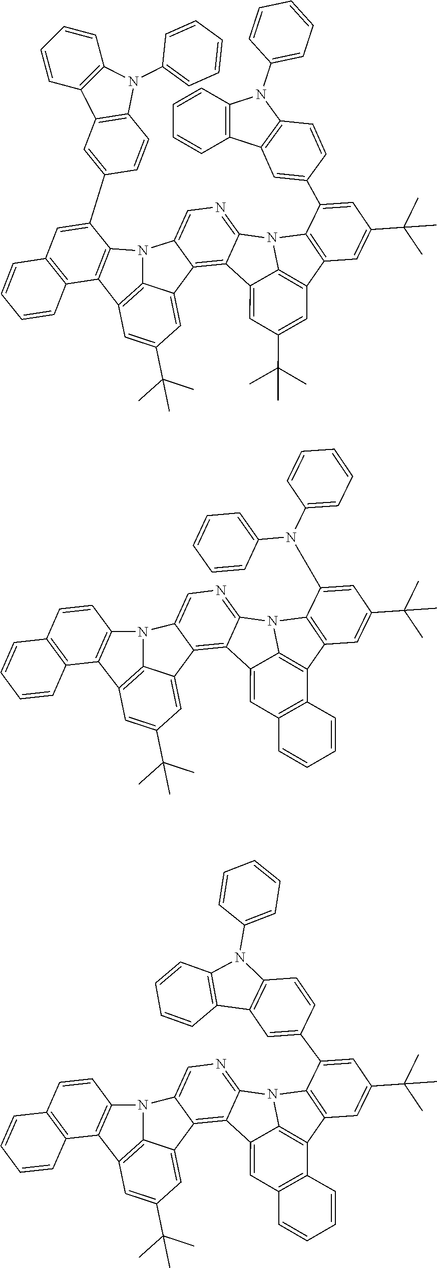 [SMILES: CC(C)(C)c1cc(-c2ccc3c(c2)c2ccccc2n3-c2ccccc2)c2c(c1)c1cc(C(C)(C)C)cc3c4c5c6cc(C(C)(C)C)cc7c8c9ccccc9cc(-c9ccc%10c(c9)c9ccccc9n%10-c9ccccc9)c8n(c5cnc4n2c13)c67 ; CC(C)(C)c1cc(N(c2ccccc2)c2ccccc2)c2c(c1)c1c3ccccc3cc3c4c5c6cc(C(C)(C)C)cc7c8c9ccccc9ccc8n(c5cnc4n2c31)c76 ; CC(C)(C)c1cc(-c2ccc3c(c2)c2ccccc2n3-c2ccccc2)c2c(c1)c1c3ccccc3cc3c4c5c6cc(C(C)(C)C)cc7c8c9ccccc9ccc8n(c5cnc4n2c31)c76]

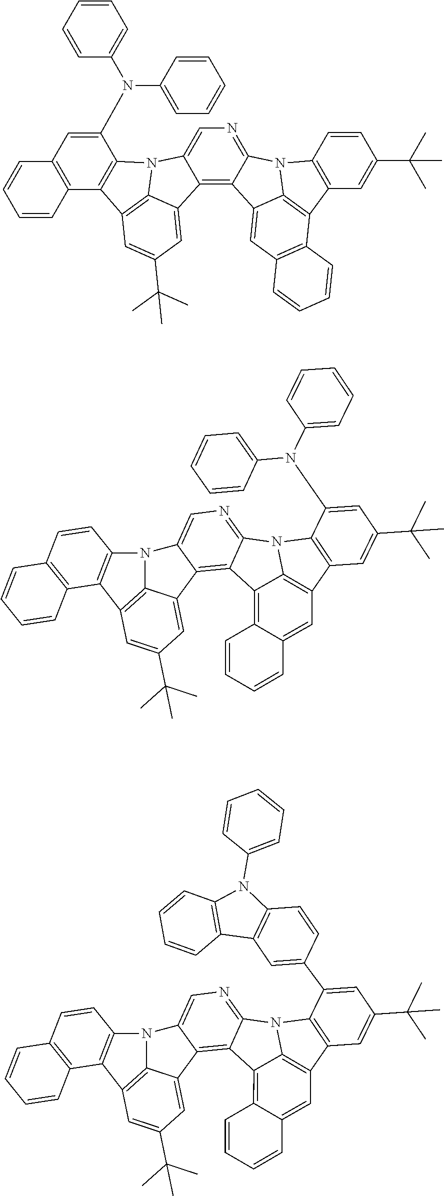 [SMILES: CC(C)(C)c1ccc2c(c1)c1c3ccccc3cc3c4c5c6cc(C(C)(C)C)cc7c8c9ccccc9cc(N(c9ccccc9)c9ccccc9)c8n(c5cnc4n2c31)c67 ; CC(C)(C)c1cc(N(c2ccccc2)c2ccccc2)c2c(c1)c1cc3ccccc3c3c4c5c6cc(C(C)(C)C)cc7c8c9ccccc9ccc8n(c5cnc4n2c13)c76 ; CC(C)(C)c1cc(-c2ccc3c(c2)c2ccccc2n3-c2ccccc2)c2c(c1)c1cc3ccccc3c3c4c5c6cc(C(C)(C)C)cc7c8c9ccccc9ccc8n(c5cnc4n2c13)c76]

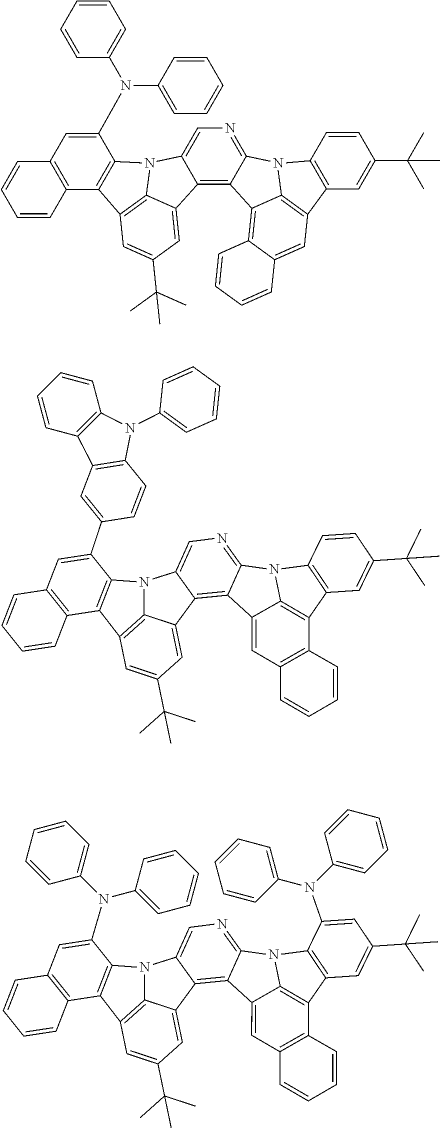 [SMILES: CC(C)(C)c1ccc2c(c1)c1cc3ccccc3c3c4c5c6cc(C(C)(C)C)cc7c8c9ccccc9cc(N(c9ccccc9)c9ccccc9)c8n(c5cnc4n2c13)c67 ; CC(C)(C)c1ccc2c(c1)c1c3ccccc3cc3c4c5c6cc(C(C)(C)C)cc7c8c9ccccc9cc(-c9ccc%10c(c9)c9ccccc9n%10-c9ccccc9)c8n(c5cnc4n2c31)c67 ; CC(C)(C)c1cc(N(c2ccccc2)c2ccccc2)c2c(c1)c1c3ccccc3cc3c4c5c6cc(C(C)(C)C)cc7c8c9ccccc9cc(N(c9ccccc9)c9ccccc9)c8n(c5cnc4n2c31)c67]

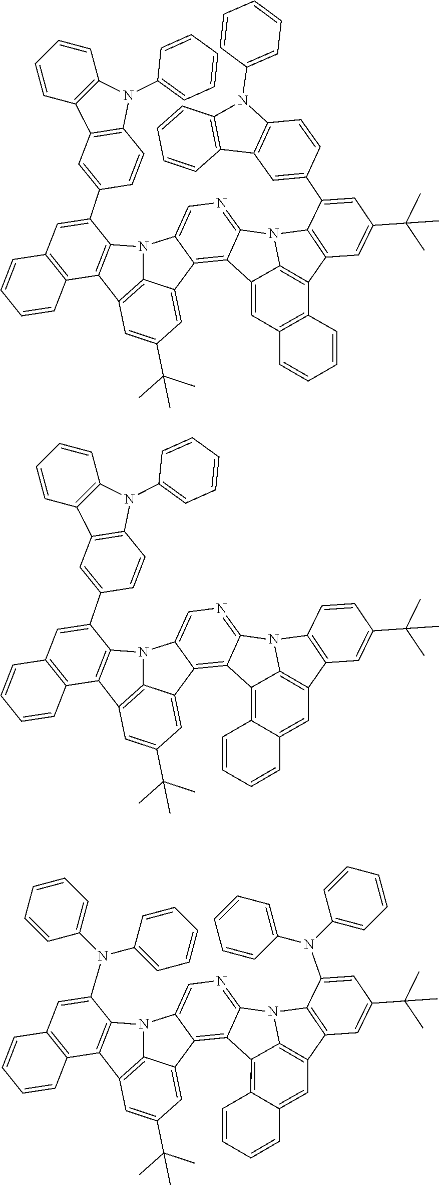

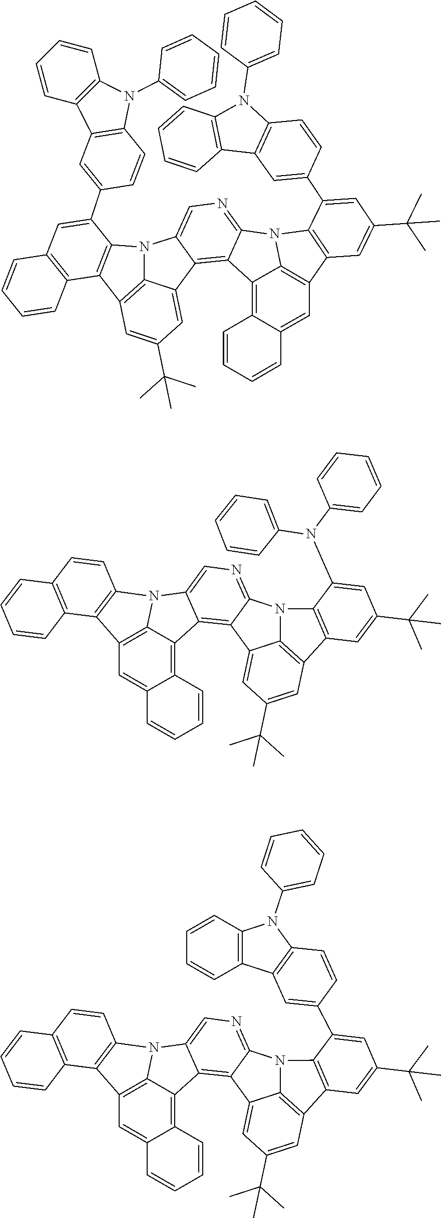 [SMILES: CC(C)(C)c1cc(-c2ccc3c(c2)c2ccccc2n3-c2ccccc2)c2c(c1)c1cc3ccccc3c3c4c5c6cc(C(C)(C)C)cc7c8c9ccccc9cc(-c9ccc%10c(c9)c9ccccc9n%10-c9ccccc9)c8n(c5cnc4n2c13)c67 ; CC(C)(C)c1cc(N(c2ccccc2)c2ccccc2)c2c(c1)c1cc(C(C)(C)C)cc3c4c5c6c7ccccc7cc7c8c9ccccc9ccc8n(c5cnc4n2c13)c76 ; CC(C)(C)c1cc(-c2ccc3c(c2)c2ccccc2n3-c2ccccc2)c2c(c1)c1cc(C(C)(C)C)cc3c4c5c6c7ccccc7cc7c8c9ccccc9ccc8n(c5cnc4n2c13)c76]

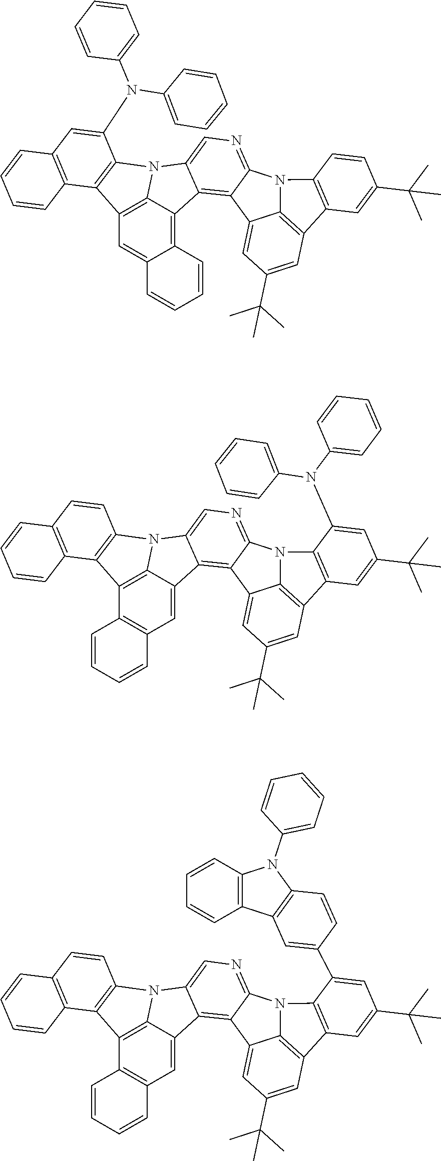 [SMILES: CC(C)(C)c1ccc2c(c1)c1cc(C(C)(C)C)cc3c4c5c6c7ccccc7cc7c8c9ccccc9cc(N(c9ccccc9)c9ccccc9)c8n(c5cnc4n2c13)c76 ; CC(C)(C)c1cc(N(c2ccccc2)c2ccccc2)c2c(c1)c1cc(C(C)(C)C)cc3c4c5c6cc7ccccc7c7c8c9ccccc9ccc8n(c5cnc4n2c13)c67 ; CC(C)(C)c1cc(-c2ccc3c(c2)c2ccccc2n3-c2ccccc2)c2c(c1)c1cc(C(C)(C)C)cc3c4c5c6cc7ccccc7c7c8c9ccccc9ccc8n(c5cnc4n2c13)c67]

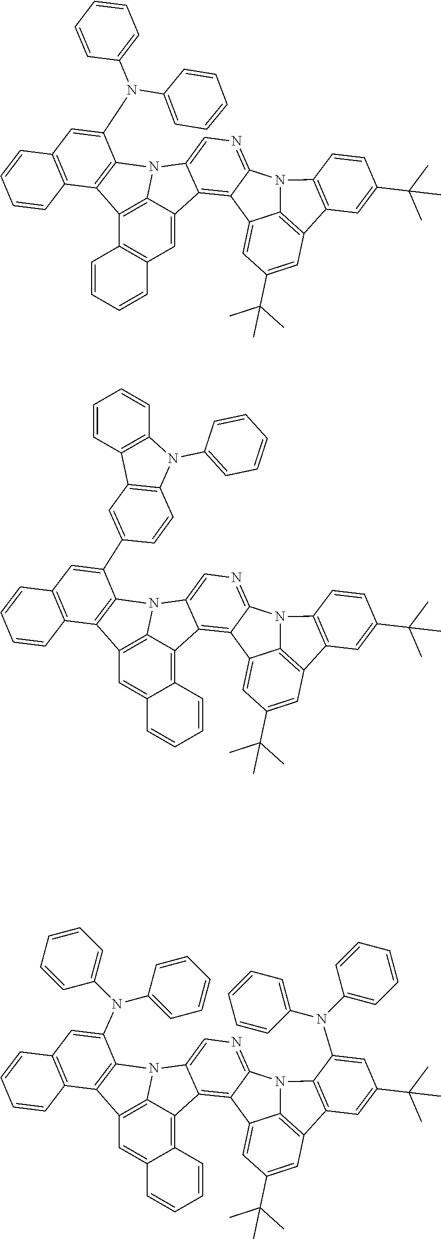 [SMILES: CC(C)(C)c1ccc2c(c1)c1cc(C(C)(C)C)cc3c4c5c6cc7ccccc7c7c8c9ccccc9cc(N(c9ccccc9)c9ccccc9)c8n(c5cnc4n2c13)c67 ; CC(C)(C)c1ccc2c(c1)c1cc(C(C)(C)C)cc3c4c5c6c7ccccc7cc7c8c9ccccc9cc(-c9ccc%10c(c9)c9ccccc9n%10-c9ccccc9)c8n(c5cnc4n2c13)c76 ; CC(C)(C)c1cc(N(c2ccccc2)c2ccccc2)c2c(c1)c1cc(C(C)(C)C)cc3c4c5c6c7ccccc7cc7c8c9ccccc9cc(N(c9ccccc9)c9ccccc9)c8n(c5cnc4n2c13)c76]

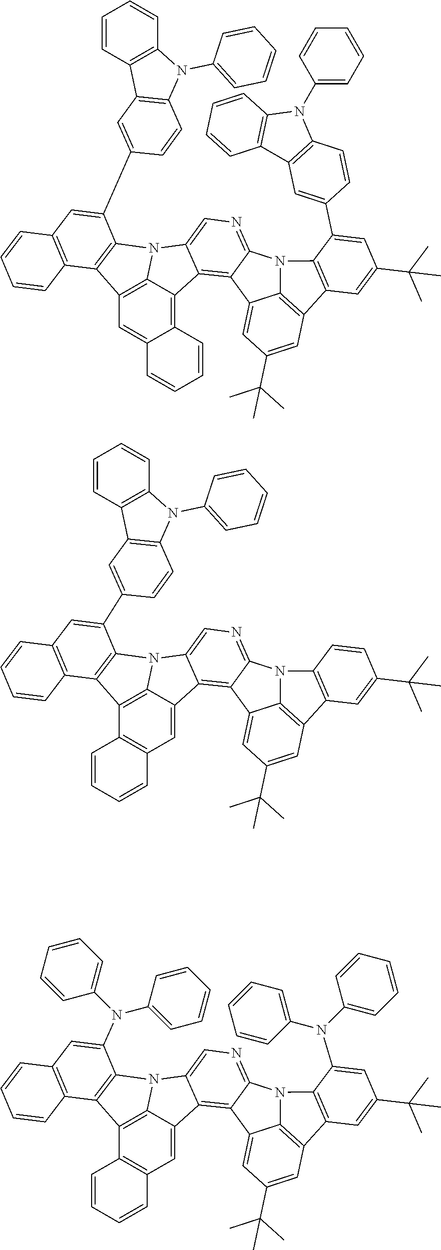 [SMILES: CC(C)(C)c1cc(-c2ccc3c(c2)c2ccccc2n3-c2ccccc2)c2c(c1)c1cc(C(C)(C)C)cc3c4c5c6c7ccccc7cc7c8c9ccccc9cc(-c9ccc%10c(c9)c9ccccc9n%10-c9ccccc9)c8n(c5cnc4n2c13)c76 ; CC(C)(C)c1ccc2c(c1)c1cc(C(C)(C)C)cc3c4c5c6cc7ccccc7c7c8c9ccccc9cc(-c9ccc%10c(c9)c9ccccc9n%10-c9ccccc9)c8n(c5cnc4n2c13)c67 ; CC(C)(C)c1cc(N(c2ccccc2)c2ccccc2)c2c(c1)c1cc(C(C)(C)C)cc3c4c5c6cc7ccccc7c7c8c9ccccc9cc(N(c9ccccc9)c9ccccc9)c8n(c5cnc4n2c13)c67]

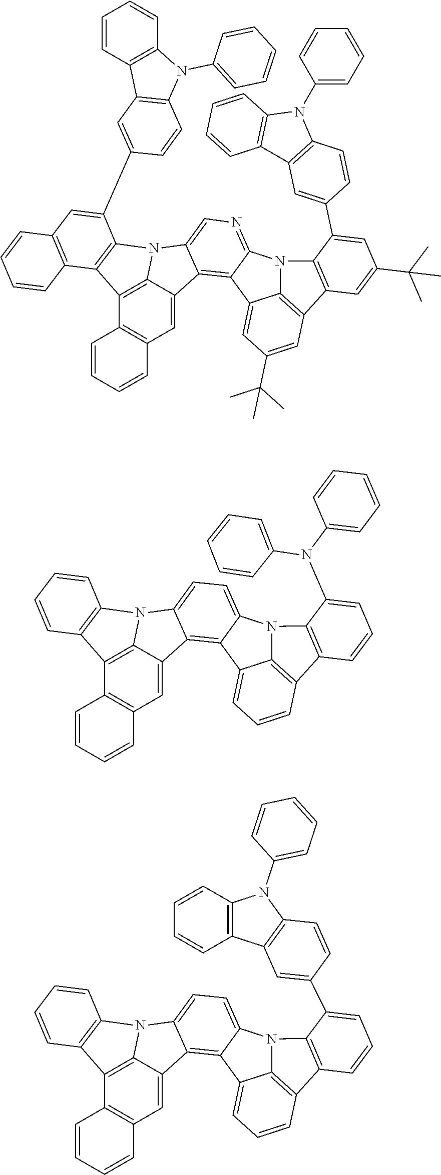 [SMILES: CC(C)(C)c1cc(-c2ccc3c(c2)c2ccccc2n3-c2ccccc2)c2c(c1)c1cc(C(C)(C)C)cc3c4c5c6cc7ccccc7c7c8c9ccccc9cc(-c9ccc%10c(c9)c9ccccc9n%10-c9ccccc9)c8n(c5cnc4n2c13)c67 ; c1ccc(N(c2ccccc2)c2cccc3c4cccc5c6c7c8cc9ccccc9c9c%10ccccc%10n(c7ccc6n(c23)c45)c89)cc1 ; c1ccc(-n2c3ccccc3c3cc(-c4cccc5c6cccc7c8c9c%10cc%11ccccc%11c%11c%12ccccc%12n(c9ccc8n(c45)c67)c%10%11)ccc32)cc1]

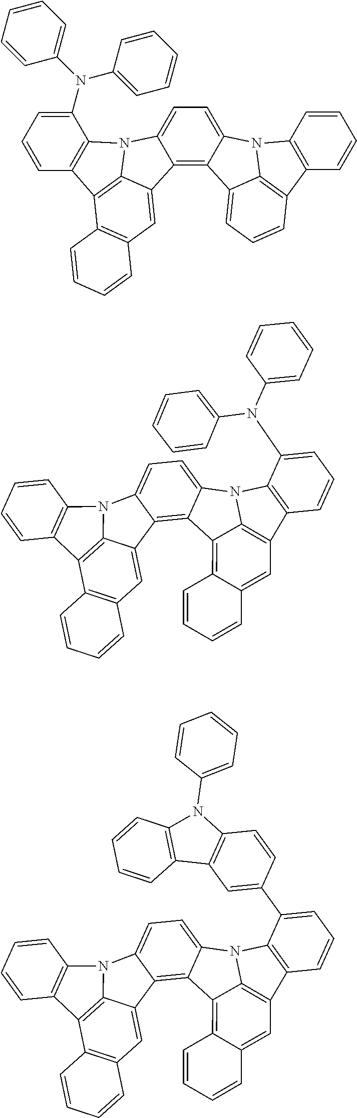 [SMILES: c1ccc(N(c2ccccc2)c2cccc3c4c5ccccc5cc5c6c7c8cccc9c%10ccccc%10n(c7ccc6n(c23)c54)c98)cc1 ; c1ccc(N(c2ccccc2)c2cccc3c4cc5ccccc5c5c6c7c8cc9ccccc9c9c%10ccccc%10n(c7ccc6n(c23)c45)c89)cc1 ; c1ccc(-n2c3ccccc3c3cc(-c4cccc5c6cc7ccccc7c7c8c9c%10cc%11ccccc%11c%11c%12ccccc%12n(c9ccc8n(c45)c67)c%10%11)ccc32)cc1]

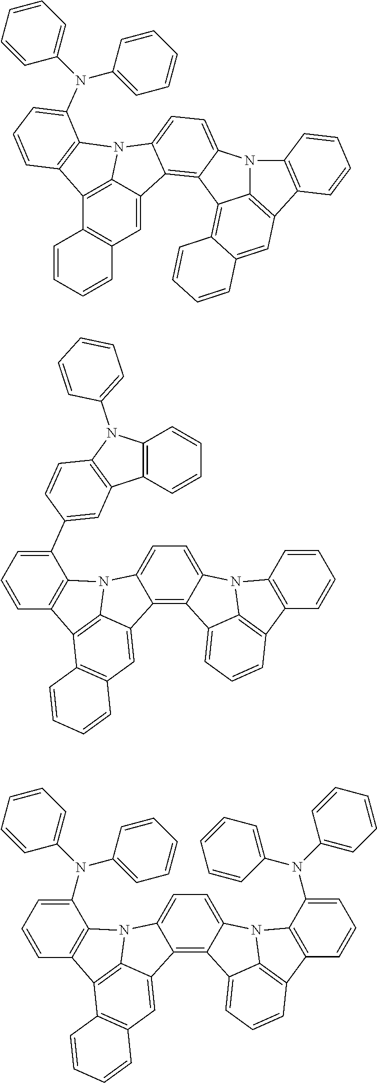

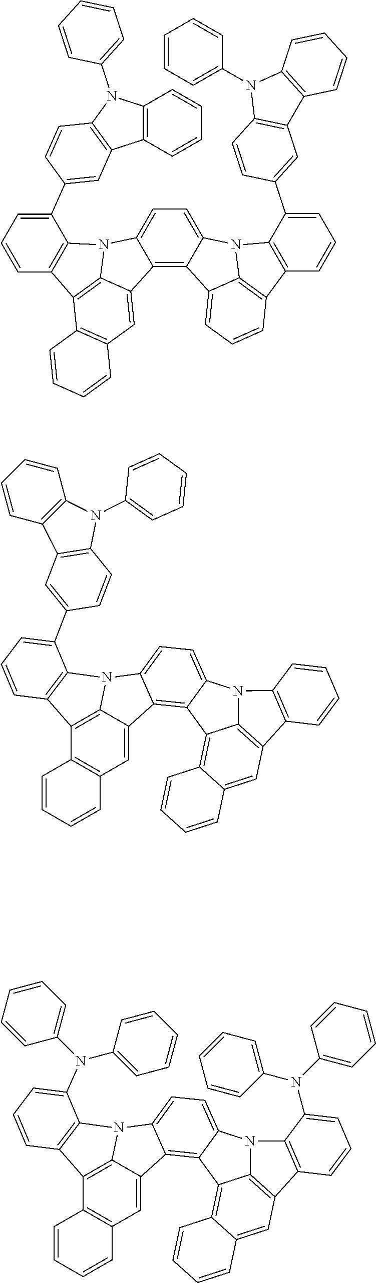 [SMILES: c1ccc(-n2c3ccccc3c3cc(-c4cccc5c6cccc7c8c9c%10cc%11ccccc%11c%11c%12cccc(-c%13ccc%14c(c%13)c%13ccccc%13n%14-c%13ccccc%13)c%12n(c9ccc8n(c45)c67)c%10%11)ccc32)cc1 ; c1ccc(-n2c3ccccc3c3cc(-c4cccc5c6c7ccccc7cc7c8c9c%10c%11ccccc%11cc%11c%12ccccc%12n(c9ccc8n(c45)c76)c%11%10)ccc32)cc1 ; c1ccc(N(c2ccccc2)c2cccc3c4c5ccccc5cc5c6c7c8c9ccccc9cc9c%10cccc(N(c%11ccccc%11)c%11ccccc%11)c%10n(c7ccc6n(c23)c54)c98)cc1]

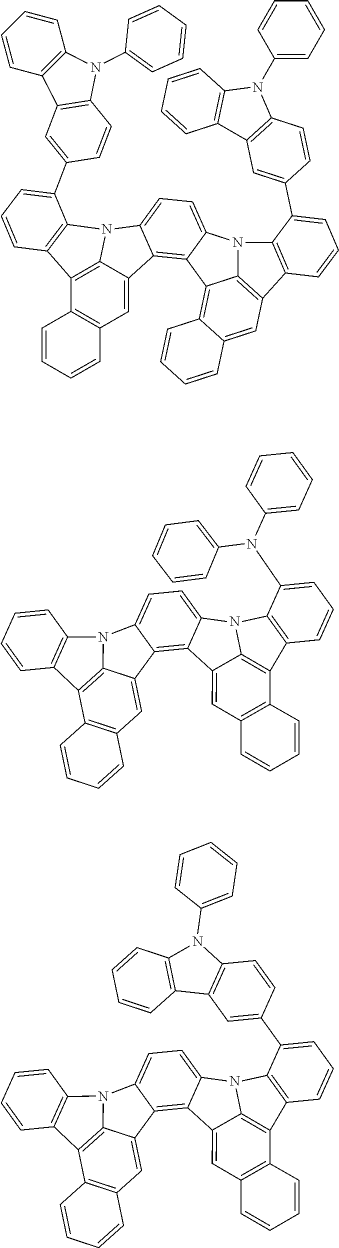 [SMILES: c1ccc(-n2c3ccccc3c3cc(-c4cccc5c6c7ccccc7cc7c8c9c%10c%11ccccc%11cc%11c%12cccc(-c%13ccc%14c(c%13)c%13ccccc%13n%14-c%13ccccc%13)c%12n(c9ccc8n(c45)c76)c%11%10)ccc32)cc1 ; c1ccc(N(c2ccccc2)c2cccc3c4c5ccccc5cc5c6c7c8cc9ccccc9c9c%10ccccc%10n(c7ccc6n(c23)c54)c89)cc1 ; c1ccc(-n2c3ccccc3c3cc(-c4cccc5c6c7ccccc7cc7c8c9c%10cc%11ccccc%11c%11c%12ccccc%12n(c9ccc8n(c45)c76)c%10%11)ccc32)cc1]

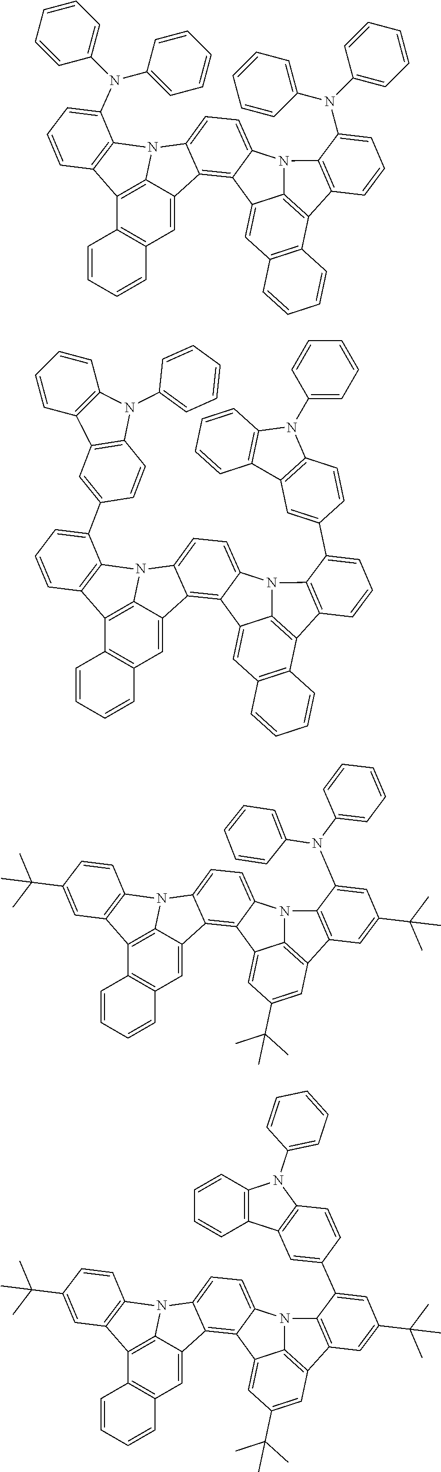

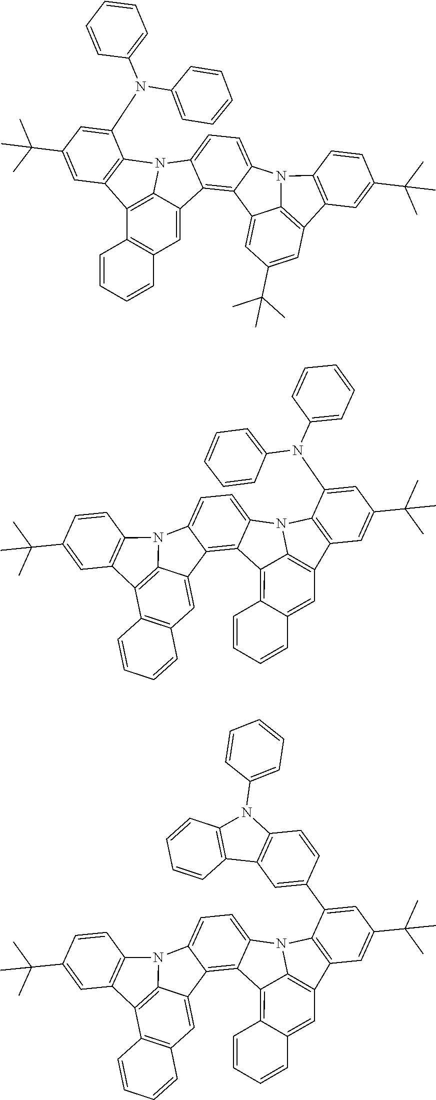

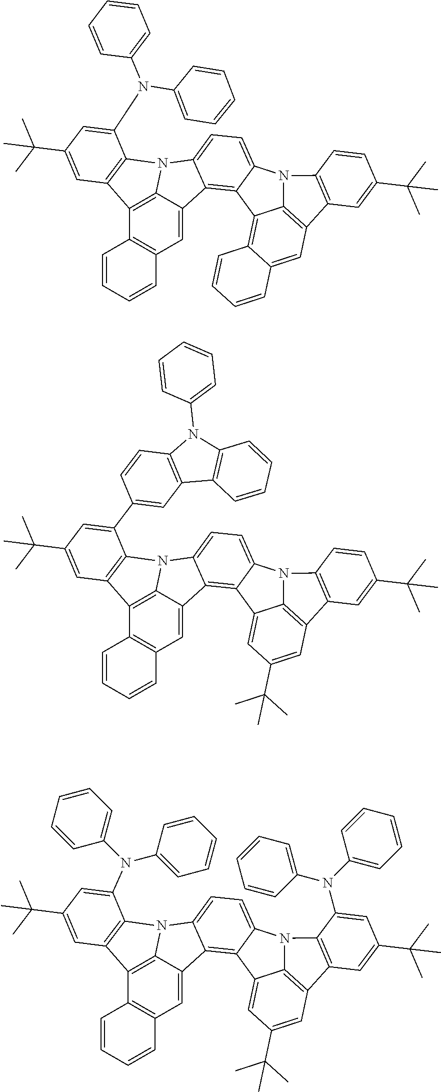

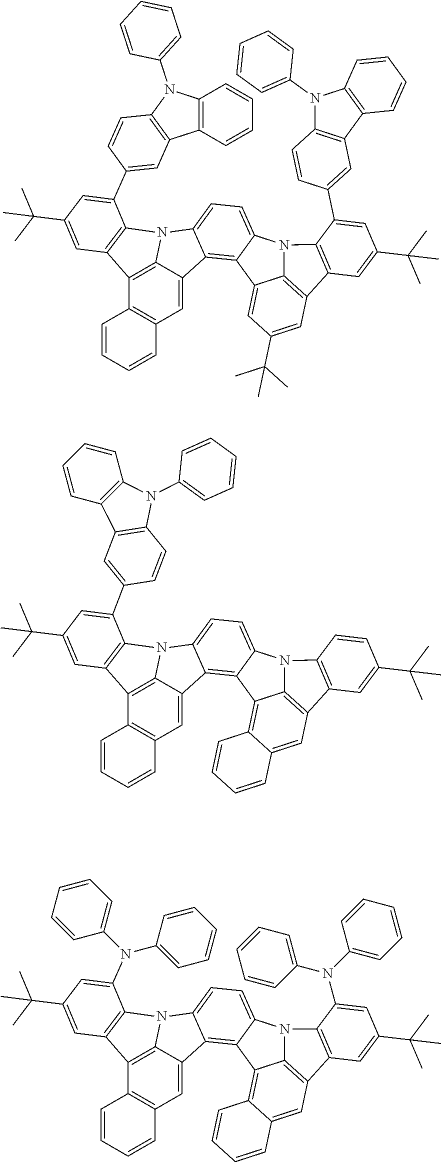

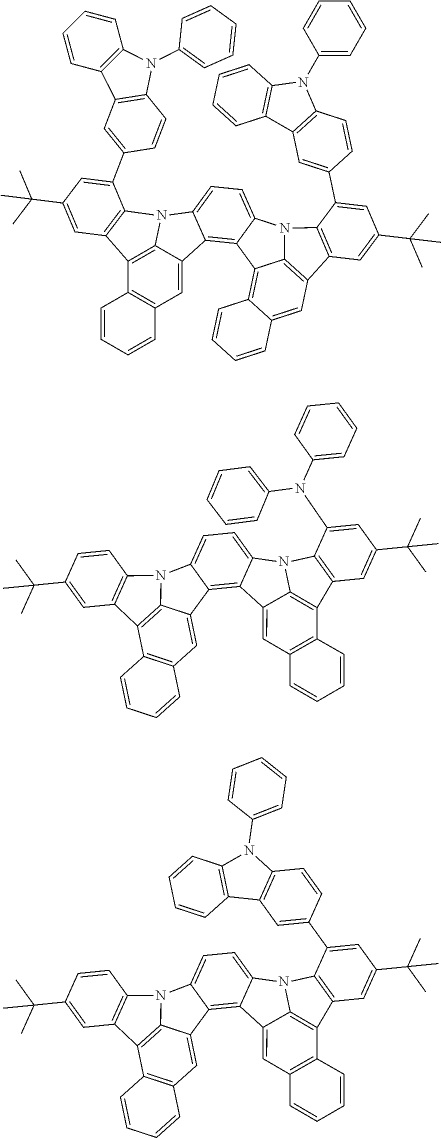

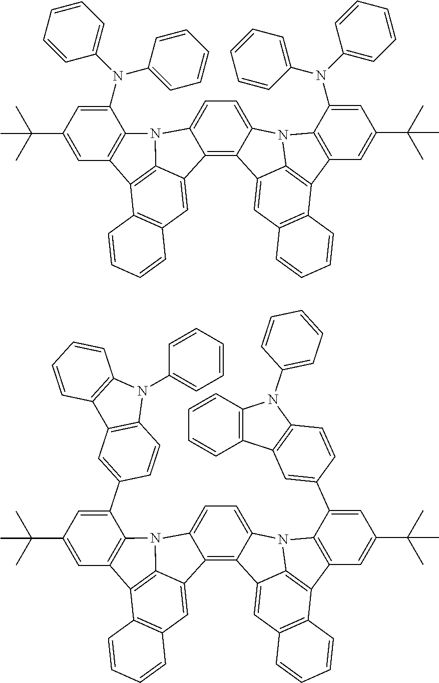

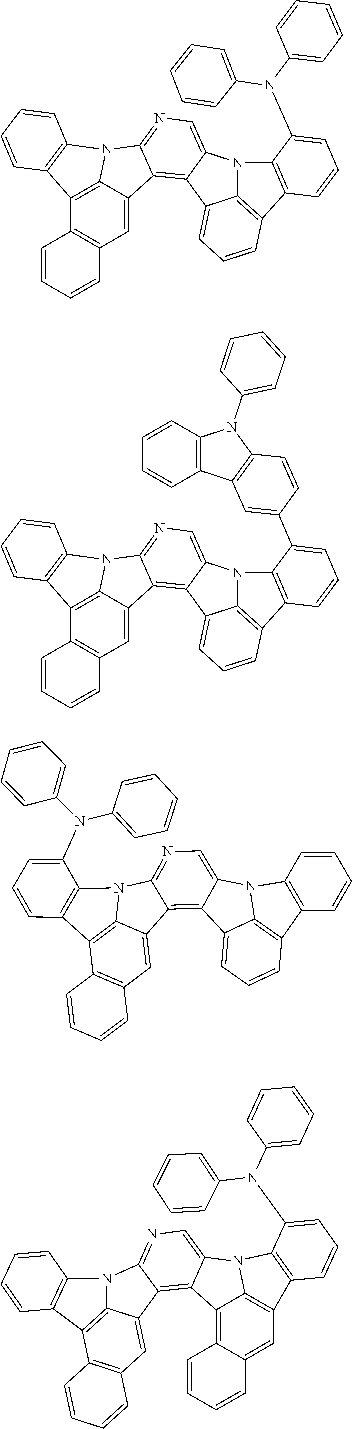

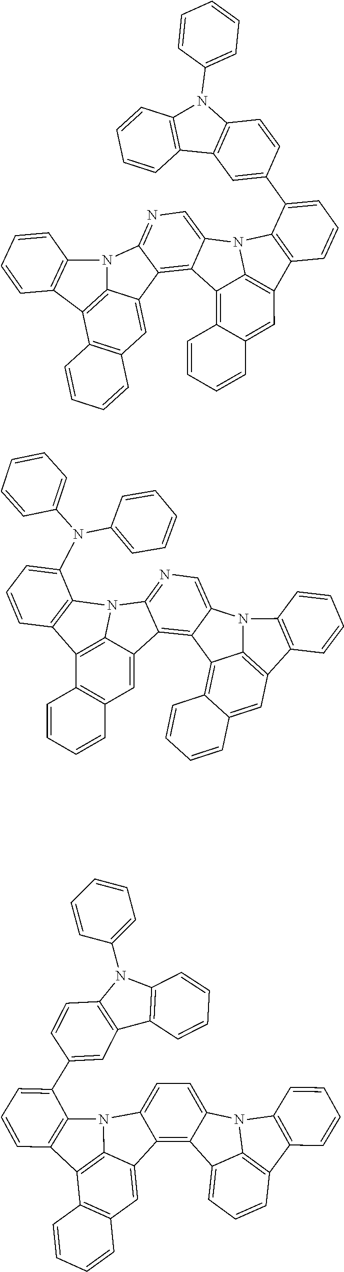 [SMILES: c1ccc(-n2c3ccccc3c3cc(-c4cccc5c6cc7ccccc7c7c8c9c%10cc%11ccccc%11c%11c%12ccccc%12n(c9ncc8n(c45)c67)c%10%11)ccc32)cc1 ; c1ccc(N(c2ccccc2)c2cccc3c4c5ccccc5cc5c6c7c8c9ccccc9cc9c%10ccccc%10n(c7cnc6n(c23)c54)c98)cc1 ; c1ccc(-n2c3ccccc3c3cc(-c4cccc5c6c7ccccc7cc7c8c9c%10cccc%11c%12ccccc%12n(c9ccc8n(c45)c76)c%11%10)ccc32)cc1]

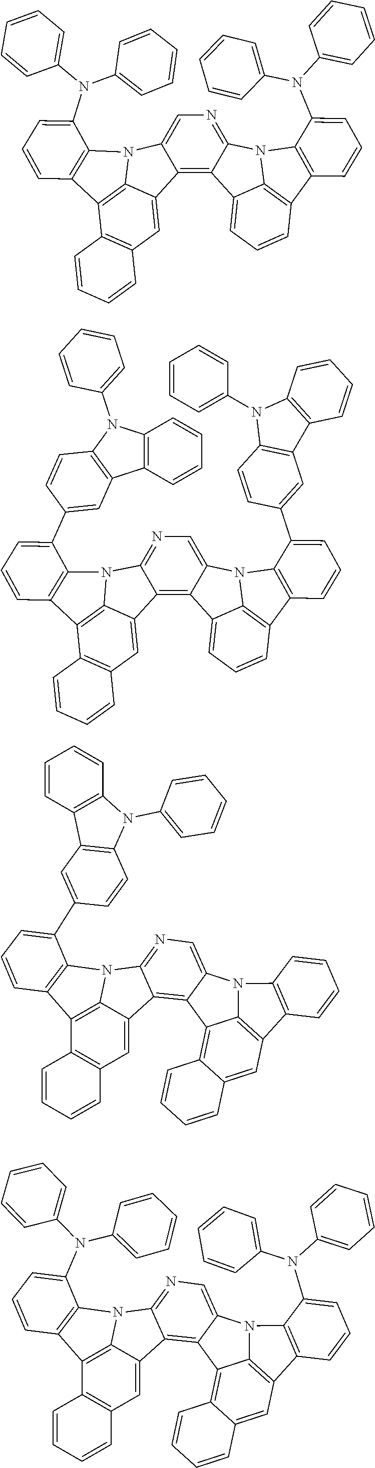

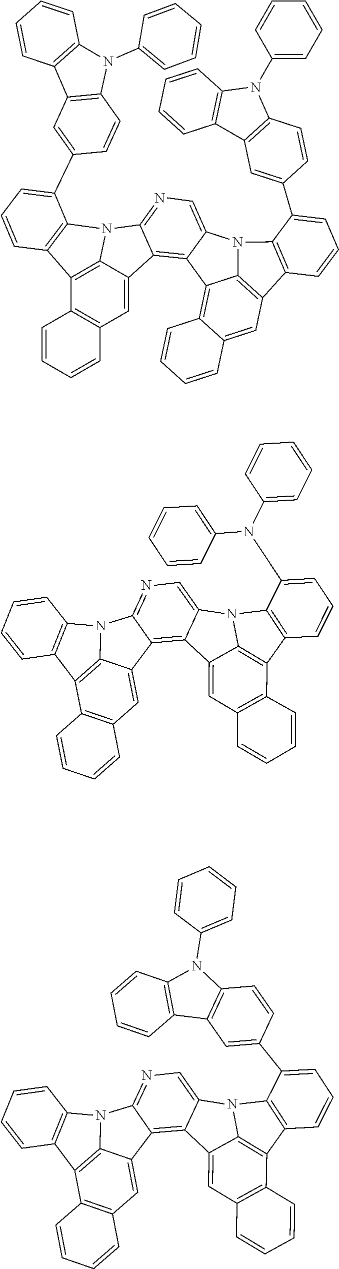 [SMILES: c1ccc(-n2c3ccccc3c3cc(-c4cccc5c6cc7ccccc7c7c8c9c%10cc%11ccccc%11c%11c%12cccc(-c%13ccc%14c(c%13)c%13ccccc%13n%14-c%13ccccc%13)c%12n(c9ncc8n(c45)c67)c%10%11)ccc32)cc1 ; c1ccc(N(c2ccccc2)c2cccc3c4c5ccccc5cc5c6c7c8cc9ccccc9c9c%10ccccc%10n(c7ncc6n(c23)c54)c89)cc1 ; c1ccc(-n2c3ccccc3c3cc(-c4cccc5c6c7ccccc7cc7c8c9c%10cc%11ccccc%11c%11c%12ccccc%12n(c9ncc8n(c45)c76)c%10%11)ccc32)cc1]

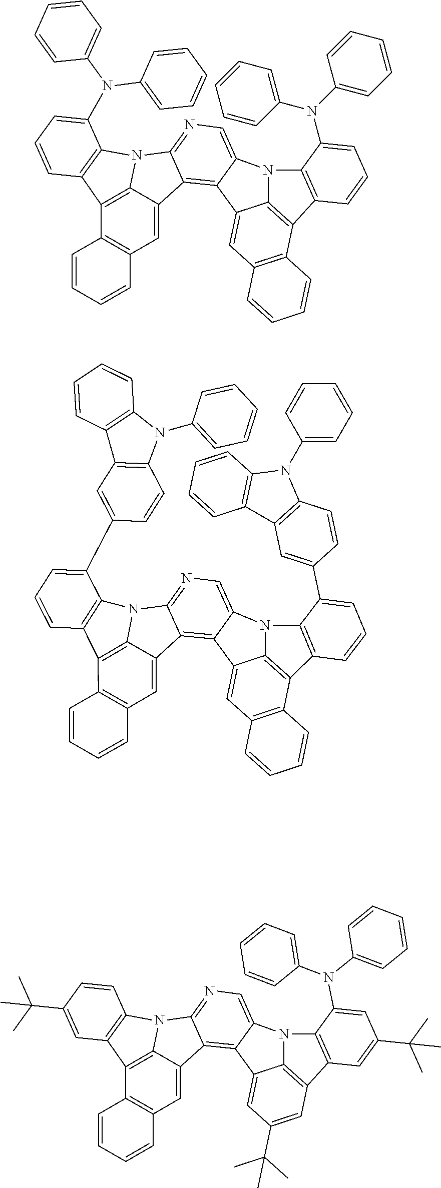 [SMILES: c1ccc(N(c2ccccc2)c2cccc3c4c5ccccc5cc5c6c7c8cc9ccccc9c9c%10cccc(N(c%11ccccc%11)c%11ccccc%11)c%10n(c7ncc6n(c23)c54)c89)cc1 ; c1ccc(-n2c3ccccc3c3cc(-c4cccc5c6c7ccccc7cc7c8c9c%10cc%11ccccc%11c%11c%12cccc(-c%13ccc%14c(c%13)c%13ccccc%13n%14-c%13ccccc%13)c%12n(c9ncc8n(c45)c76)c%10%11)ccc32)cc1 ; CC(C)(C)c1ccc2c(c1)c1c3ccccc3cc3c4c5c6cc(C(C)(C)C)cc7c8cc(C(C)(C)C)cc(N(c9ccccc9)c9ccccc9)c8n(c5cnc4n2c31)c76]

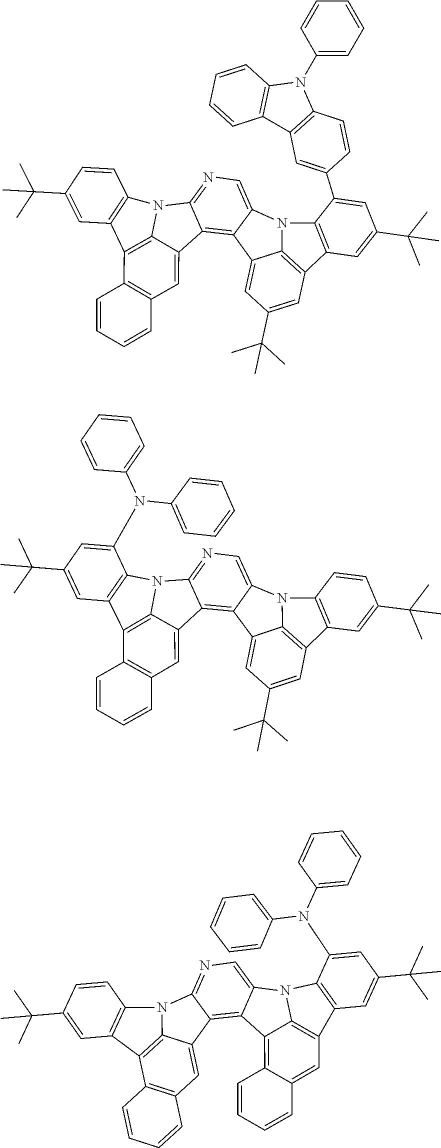

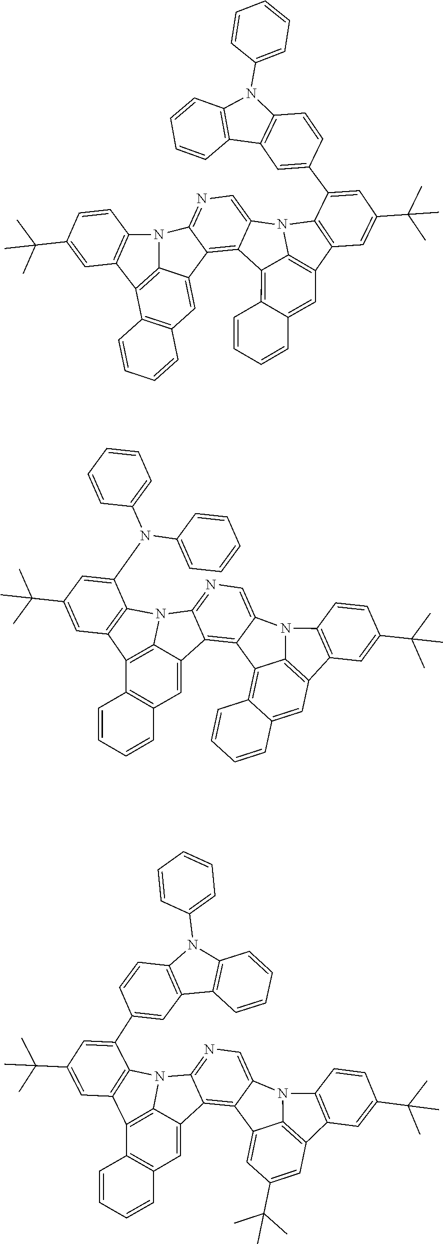

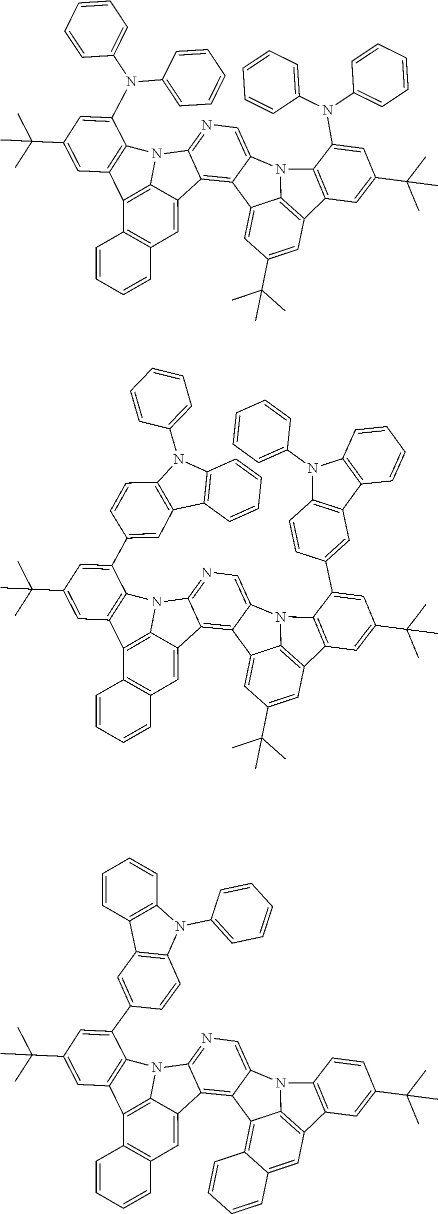

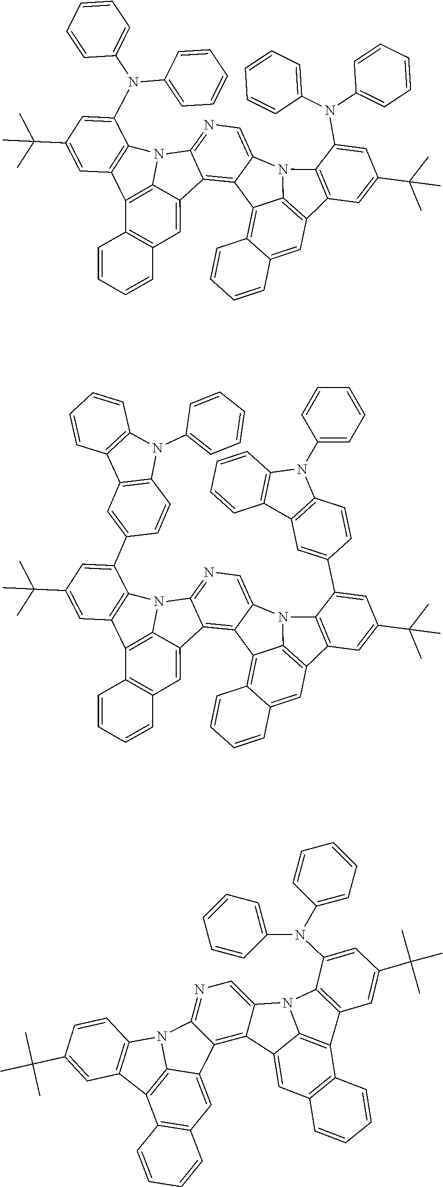

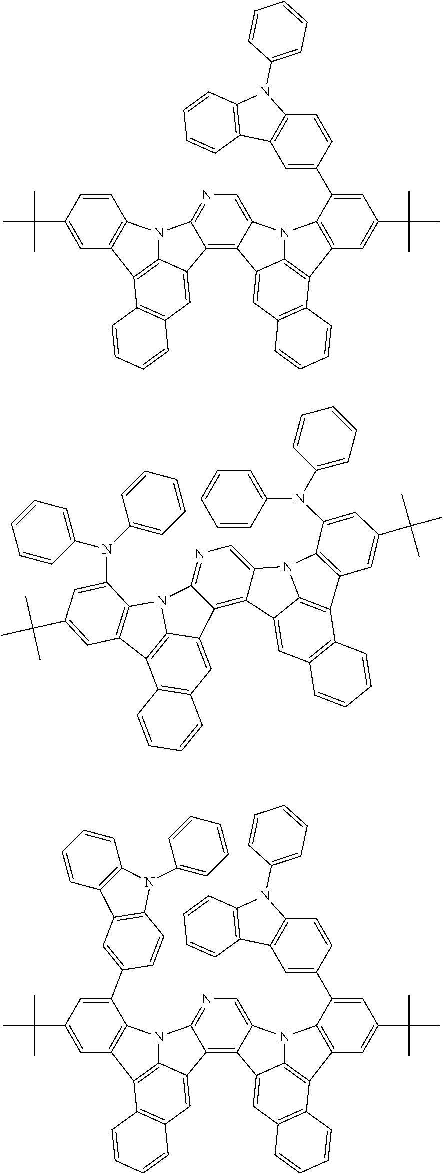

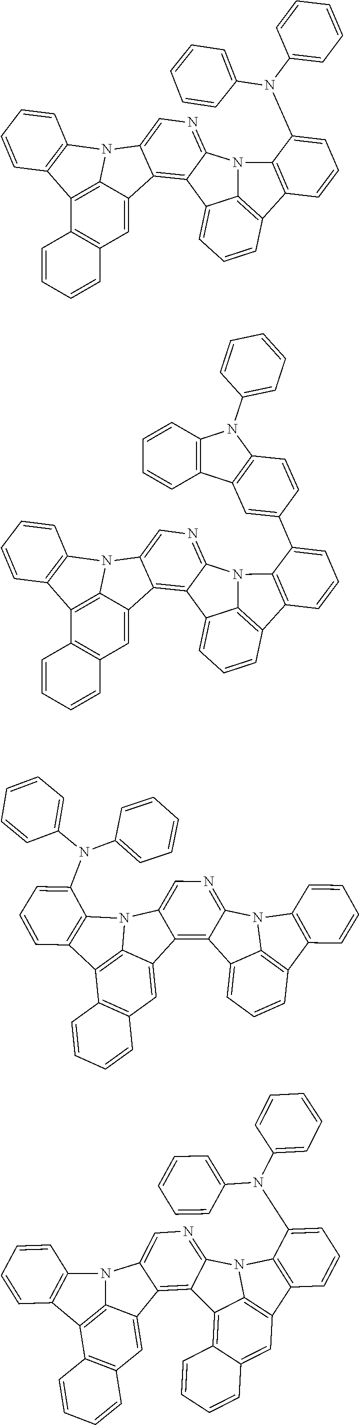 [SMILES: c1ccc(N(c2ccccc2)c2cccc3c4cccc5c6c7c8cc9ccccc9c9c%10ccccc%10n(c7cnc6n(c23)c45)c89)cc1 ; c1ccc(-n2c3ccccc3c3cc(-c4cccc5c6cccc7c8c9c%10cc%11ccccc%11c%11c%12ccccc%12n(c9cnc8n(c45)c67)c%10%11)ccc32)cc1 ; c1ccc(N(c2ccccc2)c2cccc3c4c5ccccc5cc5c6c7c8cccc9c%10ccccc%10n(c7ncc6n(c23)c54)c98)cc1 ; c1ccc(N(c2ccccc2)c2cccc3c4cc5ccccc5c5c6c7c8cc9ccccc9c9c%10ccccc%10n(c7cnc6n(c23)c45)c89)cc1]

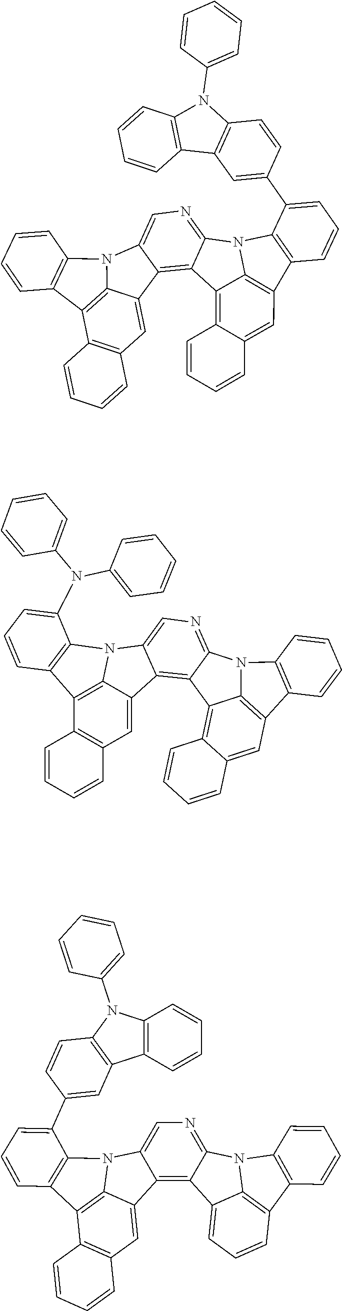 [SMILES: c1ccc(-n2c3ccccc3c3cc(-c4cccc5c6cc7ccccc7c7c8c9c%10cc%11ccccc%11c%11c%12ccccc%12n(c9cnc8n(c45)c67)c%10%11)ccc32)cc1 ; c1ccc(N(c2ccccc2)c2cccc3c4c5ccccc5cc5c6c7c8c9ccccc9cc9c%10ccccc%10n(c7ncc6n(c23)c54)c98)cc1 ; c1ccc(-n2c3ccccc3c3cc(-c4cccc5c6c7ccccc7cc7c8c9c%10cccc%11c%12ccccc%12n(c9ncc8n(c45)c76)c%11%10)ccc32)cc1]

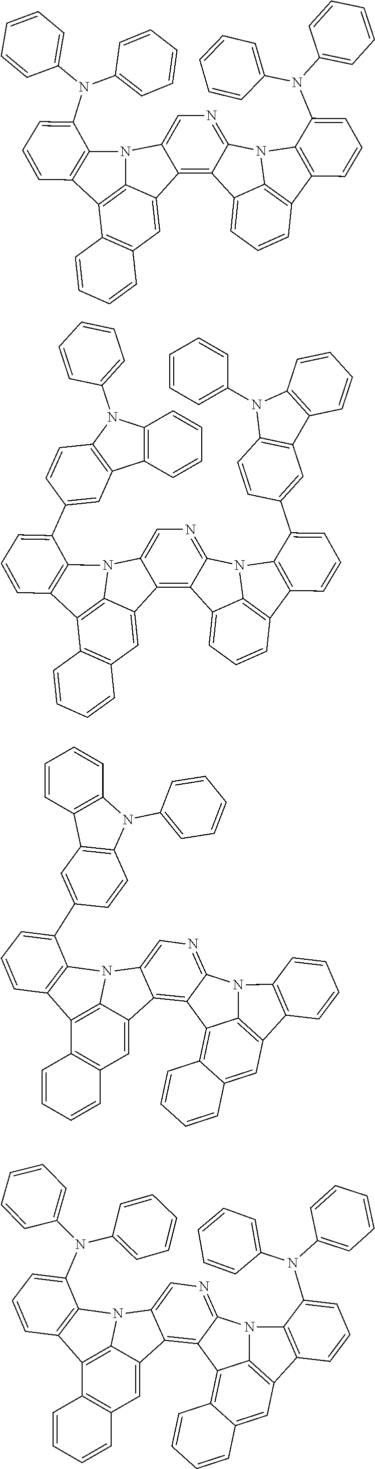

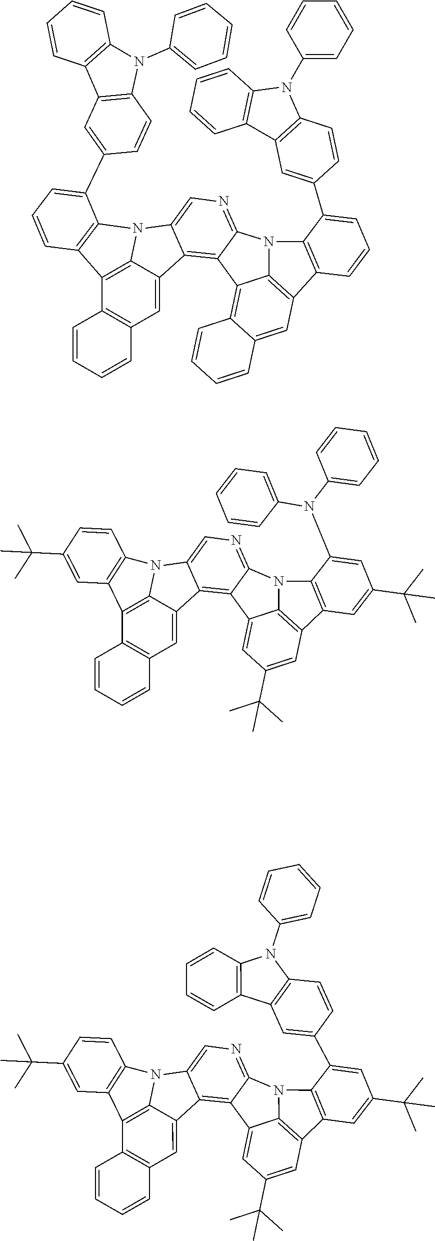

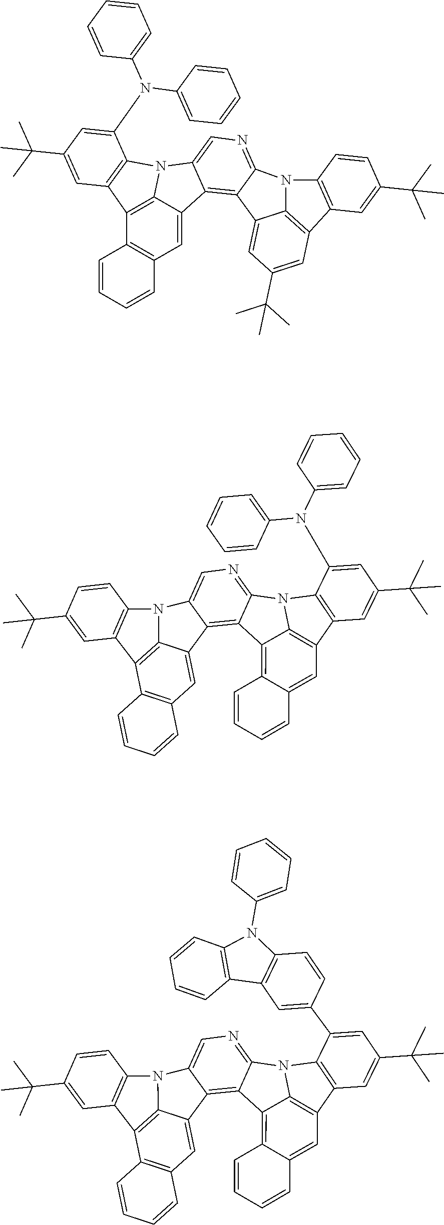

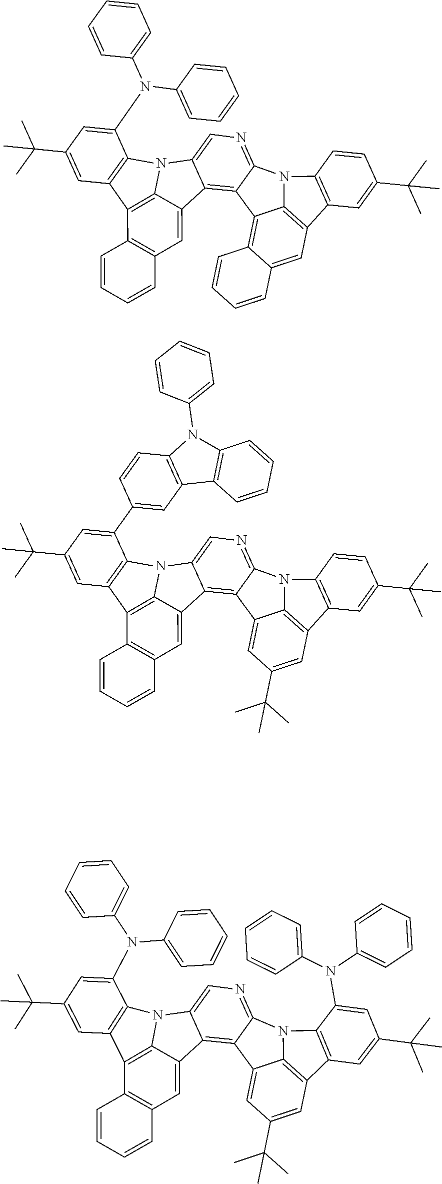

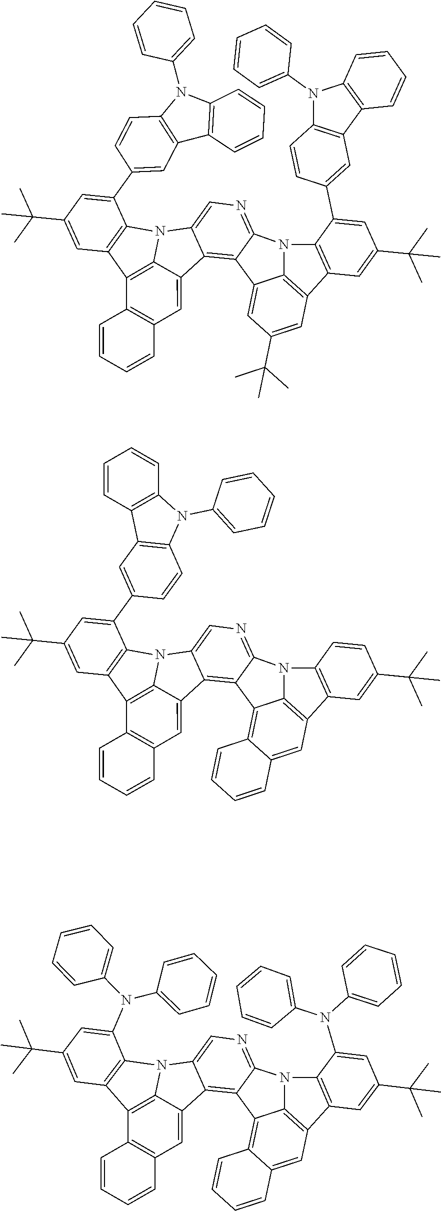

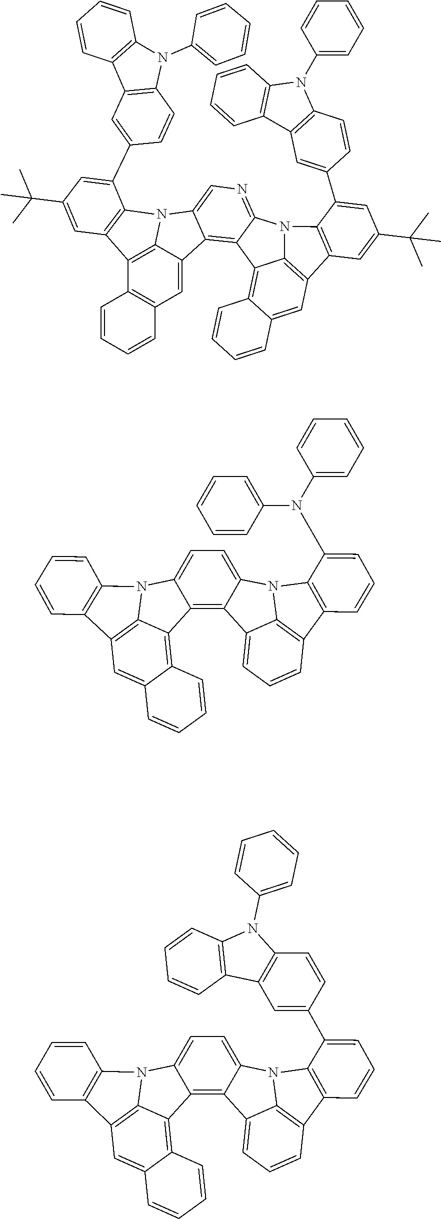 [SMILES: CC(C)(C)c1cc(-c2ccc3c(c2)c2ccccc2n3-c2ccccc2)c2c(c1)c1c3ccccc3cc3c4c5c6c7ccccc7cc7c8cc(C(C)(C)C)cc(-c9ccc%10c(c9)c9ccccc9n%10-c9ccccc9)c8n(c5ncc4n2c31)c76 ; c1ccc(N(c2ccccc2)c2cccc3c4cccc5c6c7c8c9ccccc9cc9c%10ccccc%10n(c7ccc6n(c23)c45)c98)cc1 ; c1ccc(-n2c3ccccc3c3cc(-c4cccc5c6cccc7c8c9c%10c%11ccccc%11cc%11c%12ccccc%12n(c9ccc8n(c45)c67)c%11%10)ccc32)cc1]

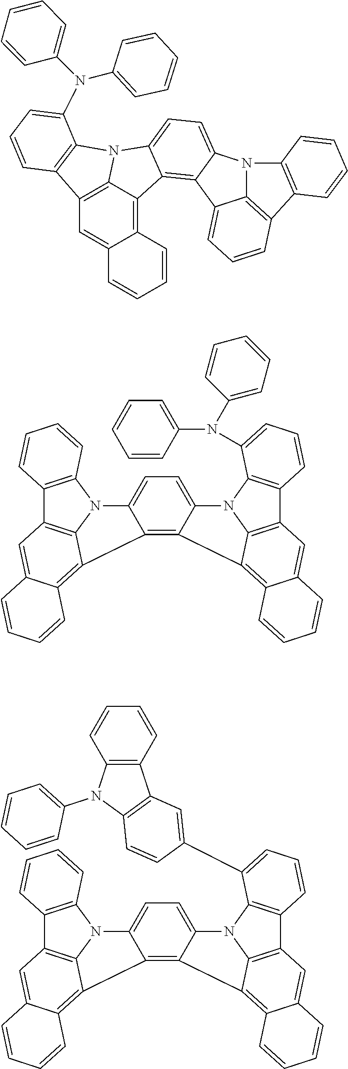 [SMILES: c1ccc(N(c2ccccc2)c2cccc3c4cc5ccccc5c5c6c7c8cccc9c%10ccccc%10n(c7ccc6n(c23)c45)c98)cc1 ; c1ccc(N(c2ccccc2)c2cccc3c4cc5ccccc5c5c6c7c8c9ccccc9cc9c%10ccccc%10n(c7ccc6n(c23)c45)c98)cc1 ; c1ccc(-n2c3ccccc3c3cc(-c4cccc5c6cc7ccccc7c7c8c9c%10c%11ccccc%11cc%11c%12ccccc%12n(c9ccc8n(c45)c67)c%11%10)ccc32)cc1]

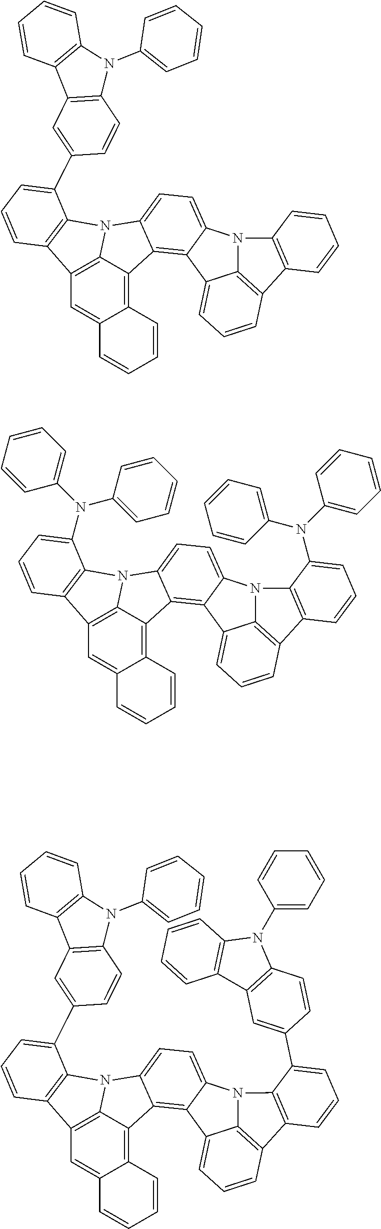 [SMILES: c1ccc(-n2c3ccccc3c3cc(-c4cccc5c6cc7ccccc7c7c8c9c%10cccc%11c%12ccccc%12n(c9ccc8n(c45)c67)c%11%10)ccc32)cc1 ; c1ccc(N(c2ccccc2)c2cccc3c4cccc5c6c7c8c9ccccc9cc9c%10cccc(N(c%11ccccc%11)c%11ccccc%11)c%10n(c7ccc6n(c23)c45)c98)cc1 ; c1ccc(-n2c3ccccc3c3cc(-c4cccc5c6cccc7c8c9c%10c%11ccccc%11cc%11c%12cccc(-c%13ccc%14c(c%13)c%13ccccc%13n%14-c%13ccccc%13)c%12n(c9ccc8n(c45)c67)c%11%10)ccc32)cc1]

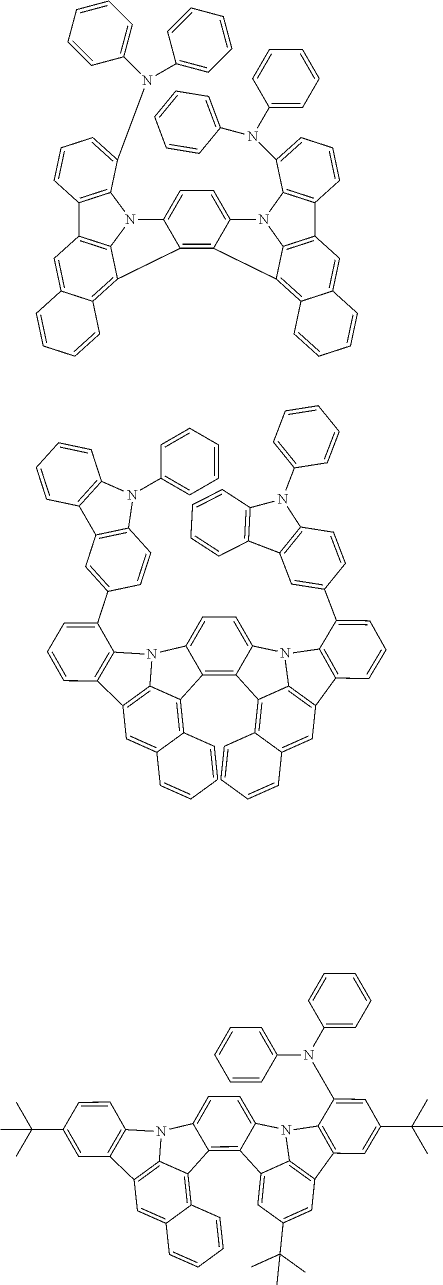 [SMILES: c1ccc(N(c2ccccc2)c2cccc3c4cc5ccccc5c5c6c7c8c9ccccc9cc9c%10cccc(N(c%11ccccc%11)c%11ccccc%11)c%10n(c7ccc6n(c23)c45)c98)cc1 ; c1ccc(-n2c3ccccc3c3cc(-c4cccc5c6cc7ccccc7c7c8c9c%10c%11ccccc%11cc%11c%12cccc(-c%13ccc%14c(c%13)c%13ccccc%13n%14-c%13ccccc%13)c%12n(c9ccc8n(c45)c67)c%11%10)ccc32)cc1 ; CC(C)(C)c1ccc2c(c1)c1cc3ccccc3c3c4c5c6cc(C(C)(C)C)cc7c8cc(C(C)(C)C)cc(N(c9ccccc9)c9ccccc9)c8n(c5ccc4n2c13)c76]

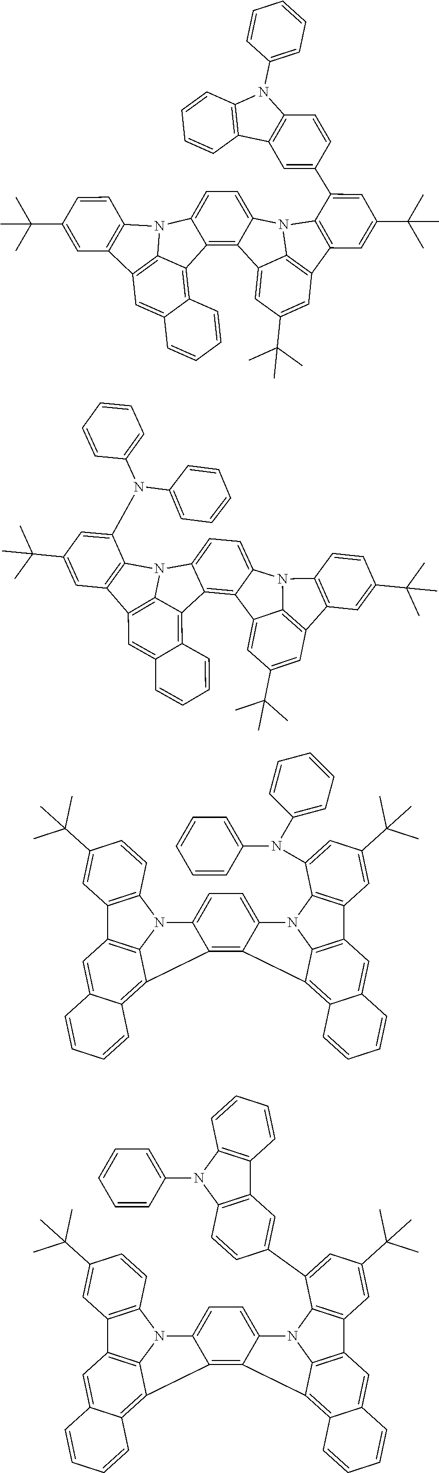

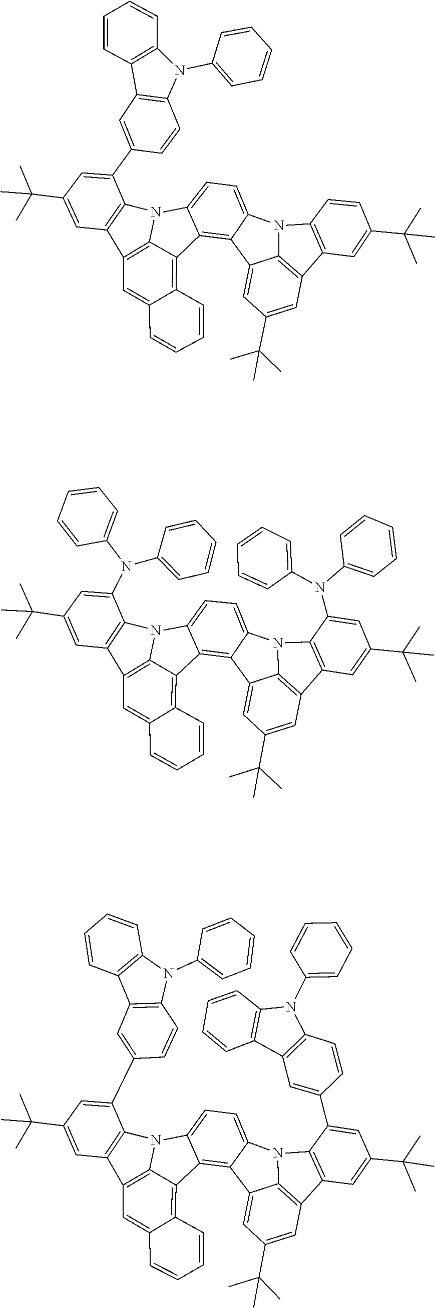

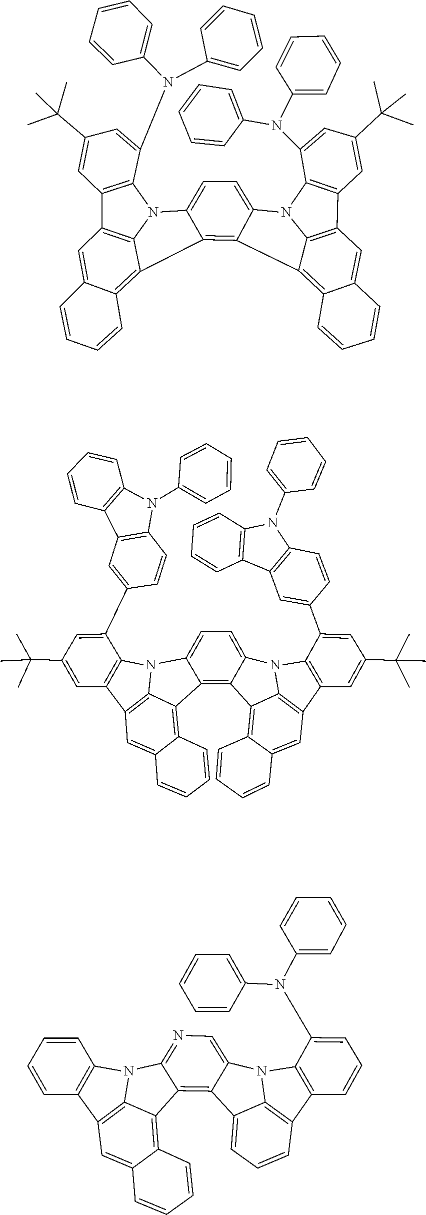

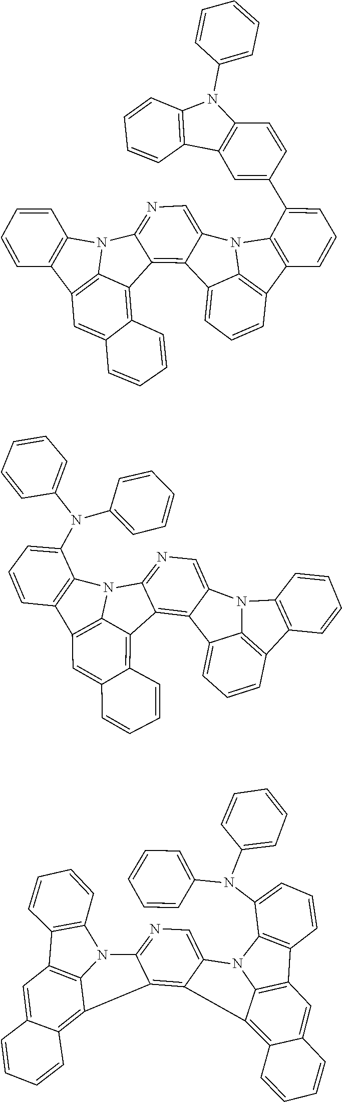 [SMILES: c1ccc(-n2c3ccccc3c3cc(-c4cccc5c6cccc7c8c9c%10c%11ccccc%11cc%11c%12ccccc%12n(c9ncc8n(c45)c67)c%11%10)ccc32)cc1 ; c1ccc(N(c2ccccc2)c2cccc3c4cc5ccccc5c5c6c7c8cccc9c%10ccccc%10n(c7cnc6n(c23)c45)c98)cc1 ; c1ccc(N(c2ccccc2)c2cccc3c4cc5ccccc5c5c6c7c8c9ccccc9cc9c%10ccccc%10n(c7ncc6n(c23)c45)c98)cc1]

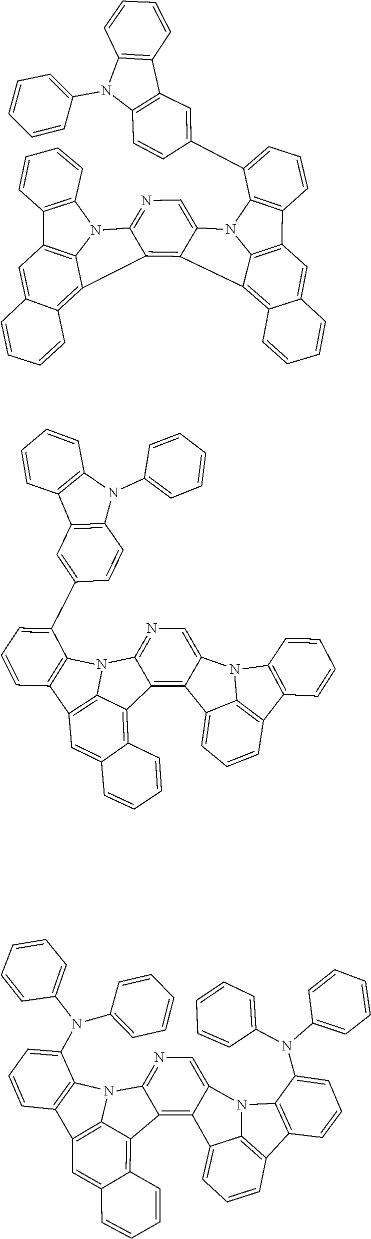 [SMILES: c1ccc(-n2c3ccccc3c3cc(-c4cccc5c6cc7ccccc7c7c8c9c%10c%11ccccc%11cc%11c%12ccccc%12n(c9ncc8n(c45)c67)c%11%10)ccc32)cc1 ; c1ccc(-n2c3ccccc3c3cc(-c4cccc5c6cc7ccccc7c7c8c9c%10cccc%11c%12ccccc%12n(c9cnc8n(c45)c67)c%11%10)ccc32)cc1 ; c1ccc(N(c2ccccc2)c2cccc3c4cccc5c6c7c8c9ccccc9cc9c%10cccc(N(c%11ccccc%11)c%11ccccc%11)c%10n(c7ncc6n(c23)c45)c98)cc1]

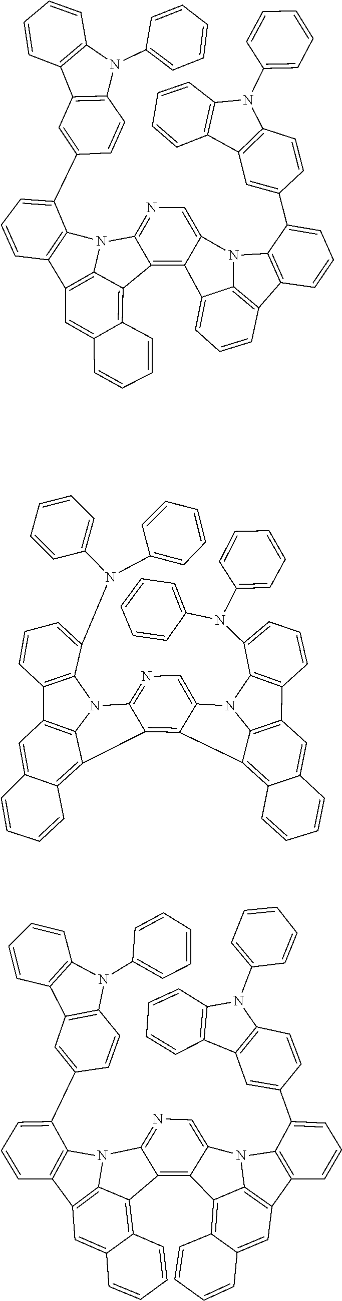 [SMILES: c1ccc(-n2c3ccccc3c3cc(-c4cccc5c6cccc7c8c9c%10c%11ccccc%11cc%11c%12cccc(-c%13ccc%14c(c%13)c%13ccccc%13n%14-c%13ccccc%13)c%12n(c9ncc8n(c45)c67)c%11%10)ccc32)cc1 ; c1ccc(N(c2ccccc2)c2cccc3c4cc5ccccc5c5c6c7c8c9ccccc9cc9c%10cccc(N(c%11ccccc%11)c%11ccccc%11)c%10n(c7ncc6n(c23)c45)c98)cc1 ; c1ccc(-n2c3ccccc3c3cc(-c4cccc5c6cc7ccccc7c7c8c9c%10c%11ccccc%11cc%11c%12cccc(-c%13ccc%14c(c%13)c%13ccccc%13n%14-c%13ccccc%13)c%12n(c9ncc8n(c45)c67)c%11%10)ccc32)cc1]

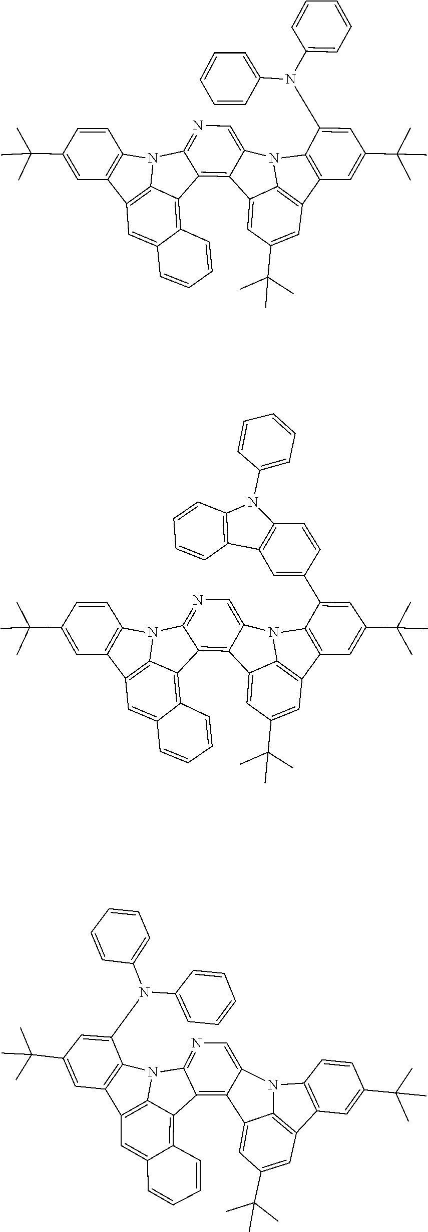

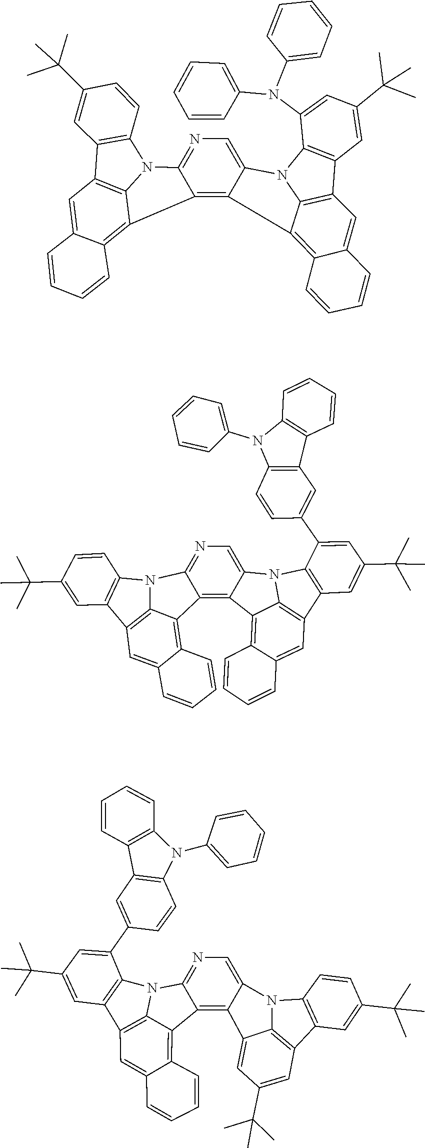

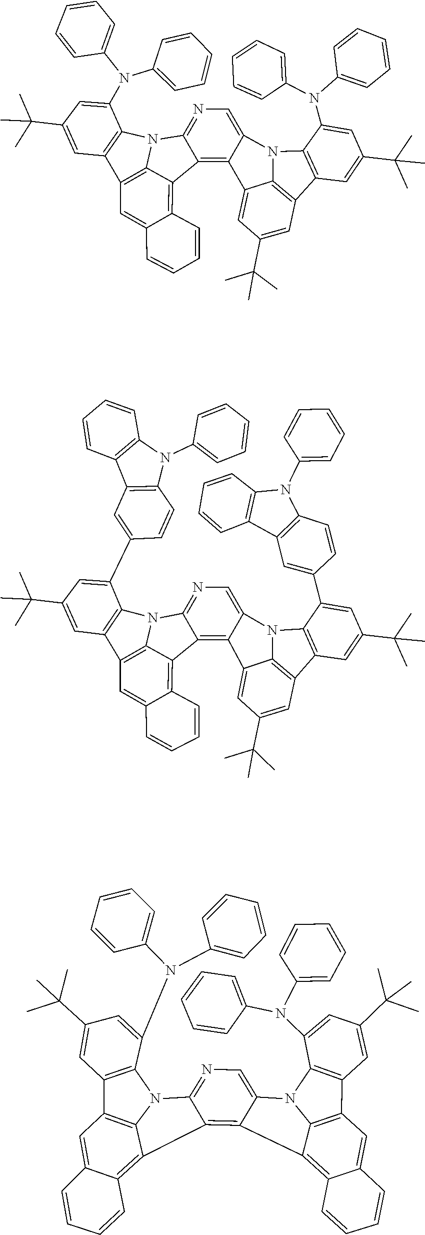

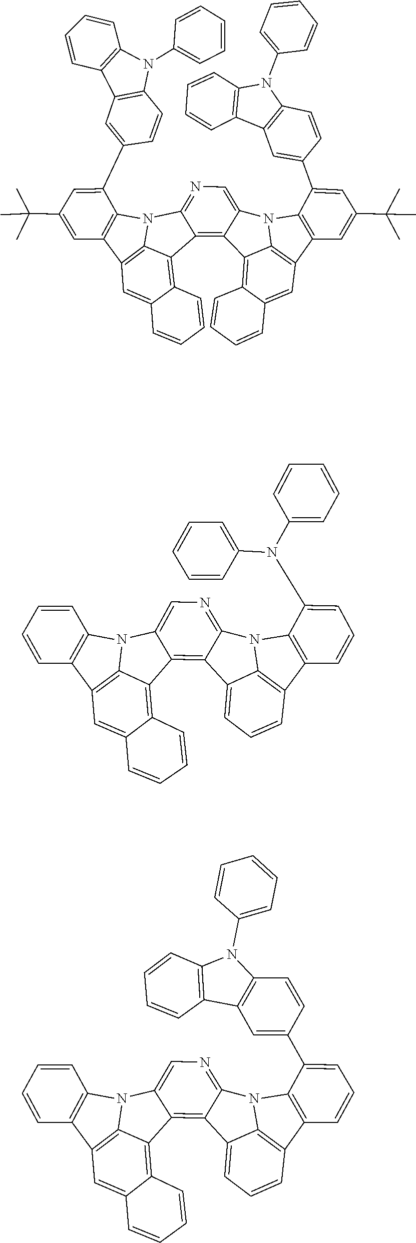 [SMILES: CC(C)(C)c1cc(-c2ccc3c(c2)c2ccccc2n3-c2ccccc2)c2c(c1)c1cc3ccccc3c3c4c5c6c7ccccc7cc7c8cc(C(C)(C)C)cc(-c9ccc%10c(c9)c9ccccc9n%10-c9ccccc9)c8n(c5ncc4n2c13)c76 ; c1ccc(N(c2ccccc2)c2cccc3c4cccc5c6c7c8c9ccccc9cc9c%10ccccc%10n(c7cnc6n(c23)c45)c98)cc1 ; c1ccc(-n2c3ccccc3c3cc(-c4cccc5c6cccc7c8c9c%10c%11ccccc%11cc%11c%12ccccc%12n(c9cnc8n(c45)c67)c%11%10)ccc32)cc1]

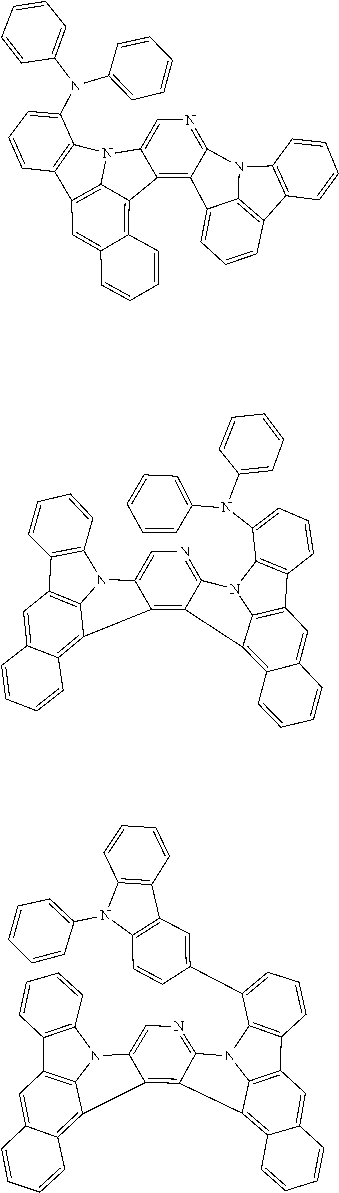 [SMILES: c1ccc(N(c2ccccc2)c2cccc3c4cc5ccccc5c5c6c7c8cccc9c%10ccccc%10n(c7ncc6n(c23)c45)c98)cc1 ; c1ccc(N(c2ccccc2)c2cccc3c4cc5ccccc5c5c6c7c8c9ccccc9cc9c%10ccccc%10n(c7cnc6n(c23)c45)c98)cc1 ; c1ccc(-n2c3ccccc3c3cc(-c4cccc5c6cc7ccccc7c7c8c9c%10c%11ccccc%11cc%11c%12ccccc%12n(c9cnc8n(c45)c67)c%11%10)ccc32)cc1]

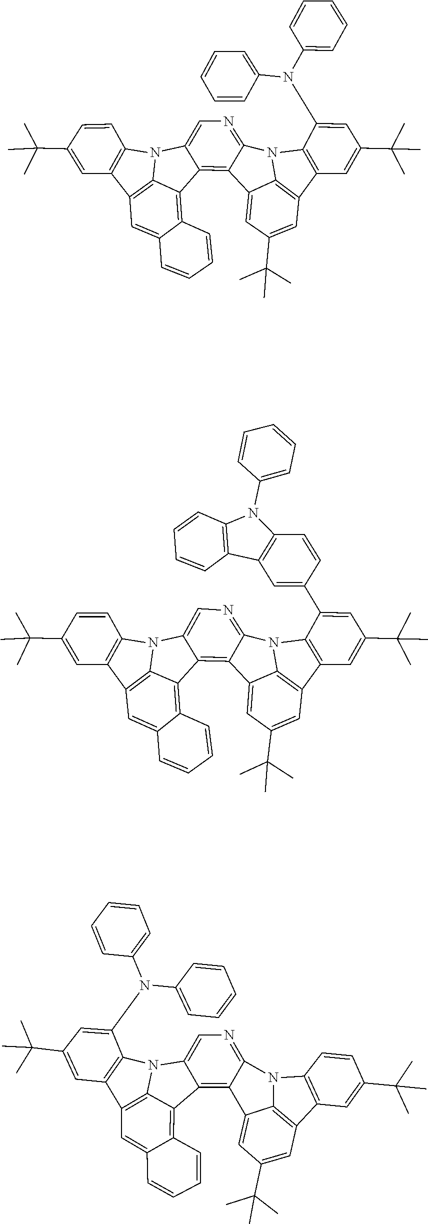

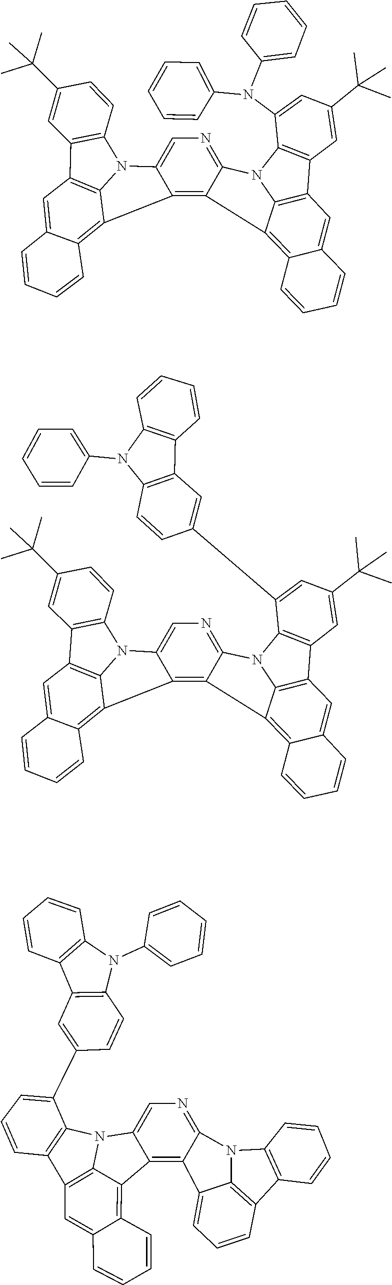

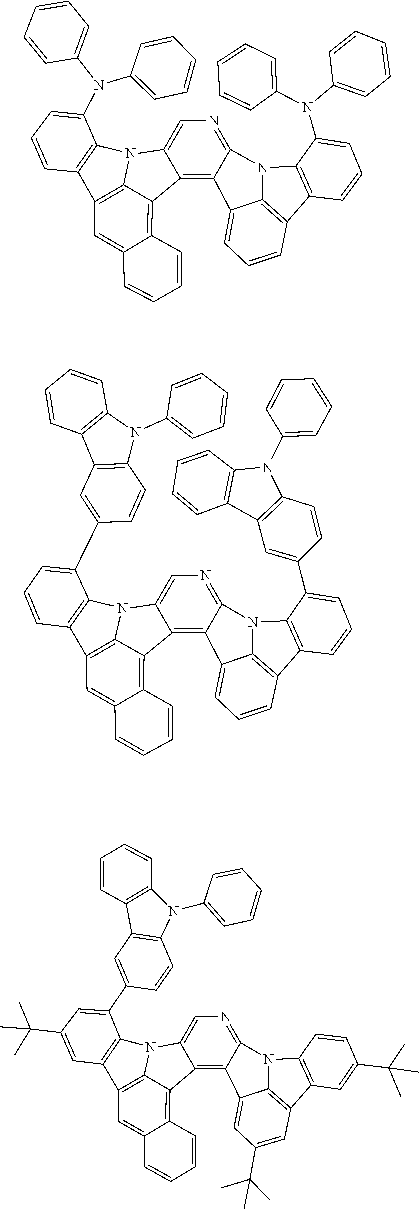 [SMILES: c1ccc(N(c2ccccc2)c2cccc3c4cc5ccccc5c5c6c7c8cccc9c%10cccc(N(c%11ccccc%11)c%11ccccc%11)c%10n(c7ncc6n(c23)c45)c98)cc1 ; c1ccc(-n2c3ccccc3c3cc(-c4cccc5c6cc7ccccc7c7c8c9c%10cccc%11c%12cccc(-c%13ccc%14c(c%13)c%13ccccc%13n%14-c%13ccccc%13)c%12n(c9ncc8n(c45)c67)c%11%10)ccc32)cc1 ; CC(C)(C)c1ccc2c(c1)c1cc(C(C)(C)C)cc3c4c5c6c7ccccc7cc7c8cc(C(C)(C)C)cc(-c9ccc%10c(c9)c9ccccc9n%10-c9ccccc9)c8n(c5cnc4n2c13)c76]

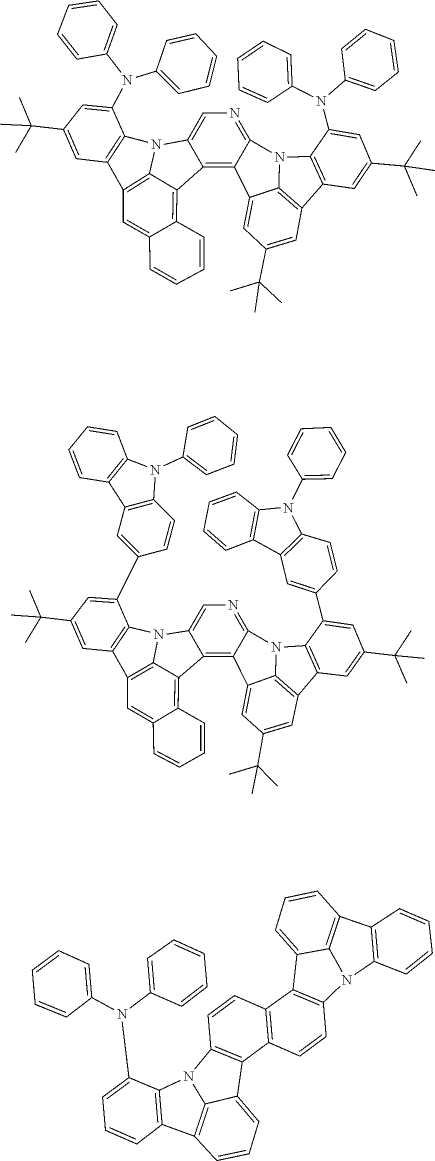

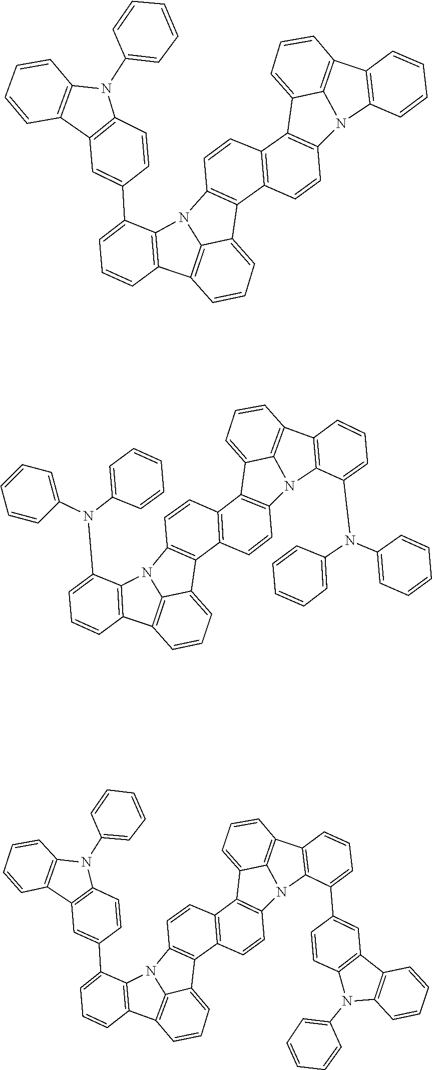 [SMILES: c1ccc(-n2c3ccccc3c3cc(-c4cccc5c6cccc7c8c9ccc%10c(c9ccc8n(c45)c67)c4cccc5c6ccccc6n%10c54)ccc32)cc1 ; c1ccc(N(c2ccccc2)c2cccc3c4cccc5c6c7ccc8c(c7ccc6n(c23)c45)c2cccc3c4cccc(N(c5ccccc5)c5ccccc5)c4n8c32)cc1 ; c1ccc(-n2c3ccccc3c3cc(-c4cccc5c6cccc7c8c9ccc%10c(c9ccc8n(c45)c67)c4cccc5c6cccc(-c7ccc8c(c7)c7ccccc7n8-c7ccccc7)c6n%10c54)ccc32)cc1]

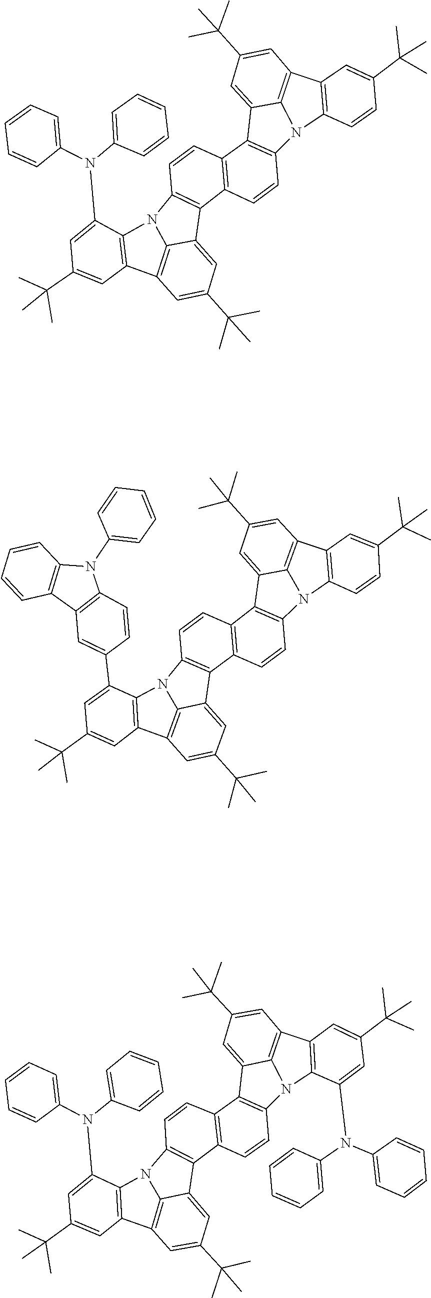 [SMILES: CC(C)(C)c1ccc2c(c1)c1cc(C(C)(C)C)cc3c4c5ccc6c(c5ccc4n2c13)c1cc(C(C)(C)C)cc2c3cc(C(C)(C)C)cc(N(c4ccccc4)c4ccccc4)c3n6c21 ; CC(C)(C)c1ccc2c(c1)c1cc(C(C)(C)C)cc3c4c5ccc6c(c5ccc4n2c13)c1cc(C(C)(C)C)cc2c3cc(C(C)(C)C)cc(-c4ccc5c(c4)c4ccccc4n5-c4ccccc4)c3n6c21 ; CC(C)(C)c1cc(N(c2ccccc2)c2ccccc2)c2c(c1)c1cc(C(C)(C)C)cc3c4c5ccc6c(c5ccc4n2c13)c1cc(C(C)(C)C)cc2c3cc(C(C)(C)C)cc(N(c4ccccc4)c4ccccc4)c3n6c21]

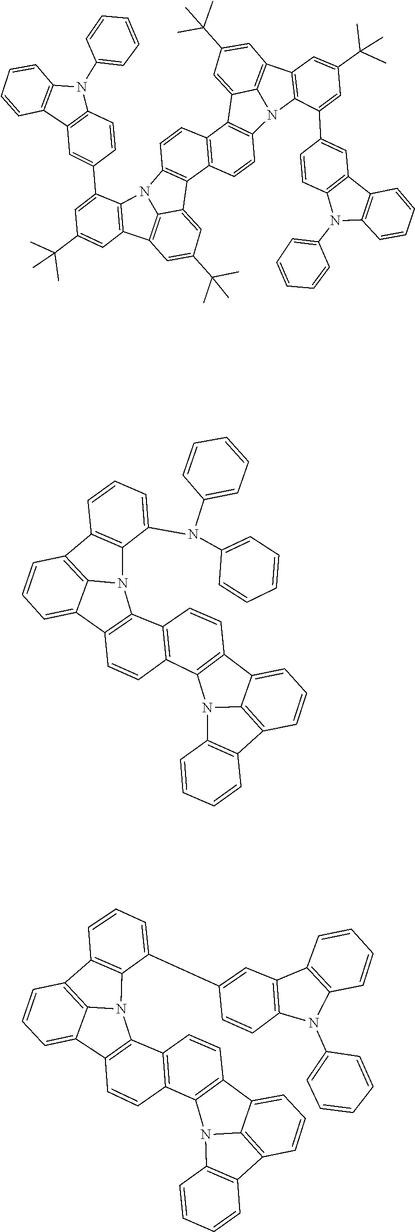 [SMILES: CC(C)(C)c1cc(-c2ccc3c(c2)c2ccccc2n3-c2ccccc2)c2c(c1)c1cc(C(C)(C)C)cc3c4c5ccc6c(c5ccc4n2c13)c1cc(C(C)(C)C)cc2c3cc(C(C)(C)C)cc(-c4ccc5c(c4)c4ccccc4n5-c4ccccc4)c3n6c21 ; c1ccc(N(c2ccccc2)c2cccc3c4cccc5c6ccc7c(ccc8c9cccc%10c%11ccccc%11n(c%109)c87)c6n(c23)c45)cc1 ; c1ccc(-n2c3ccccc3c3cc(-c4cccc5c6cccc7c8ccc9c(ccc%10c%11cccc%12c%13ccccc%13n(c%12%11)c%109)c8n(c45)c67)ccc32)cc1]

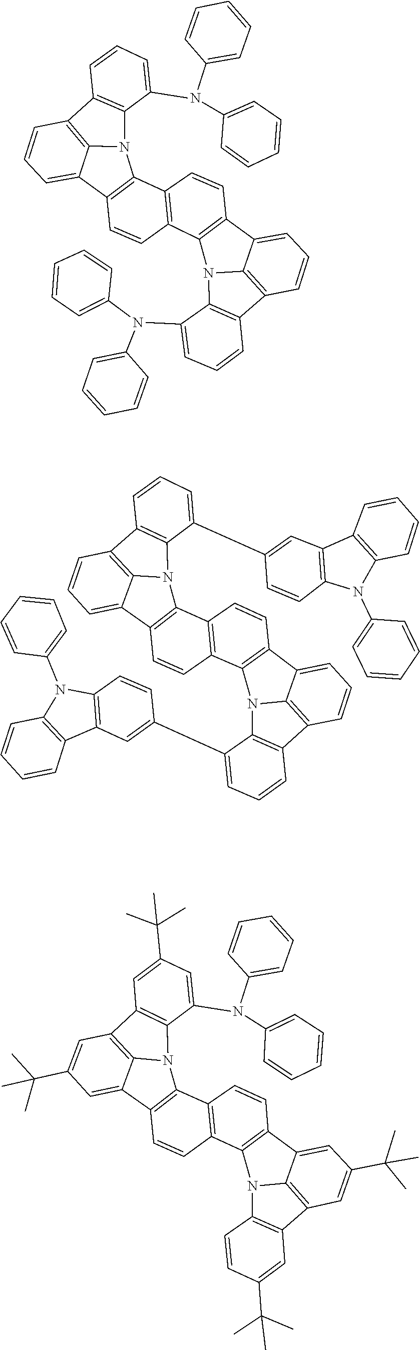 [SMILES: c1ccc(N(c2ccccc2)c2cccc3c4cccc5c6ccc7c(ccc8c9cccc%10c%11cccc(N(c%12ccccc%12)c%12ccccc%12)c%11n(c%109)c87)c6n(c23)c45)cc1 ; c1ccc(-n2c3ccccc3c3cc(-c4cccc5c6cccc7c8ccc9c(ccc%10c%11cccc%12c%13cccc(-c%14ccc%15c(c%14)c%14ccccc%14n%15-c%14ccccc%14)c%13n(c%12%11)c%109)c8n(c45)c67)ccc32)cc1 ; CC(C)(C)c1ccc2c(c1)c1cc(C(C)(C)C)cc3c4ccc5c(ccc6c7cc(C(C)(C)C)cc8c9cc(C(C)(C)C)cc(N(c%10ccccc%10)c%10ccccc%10)c9n(c87)c56)c4n2c13]

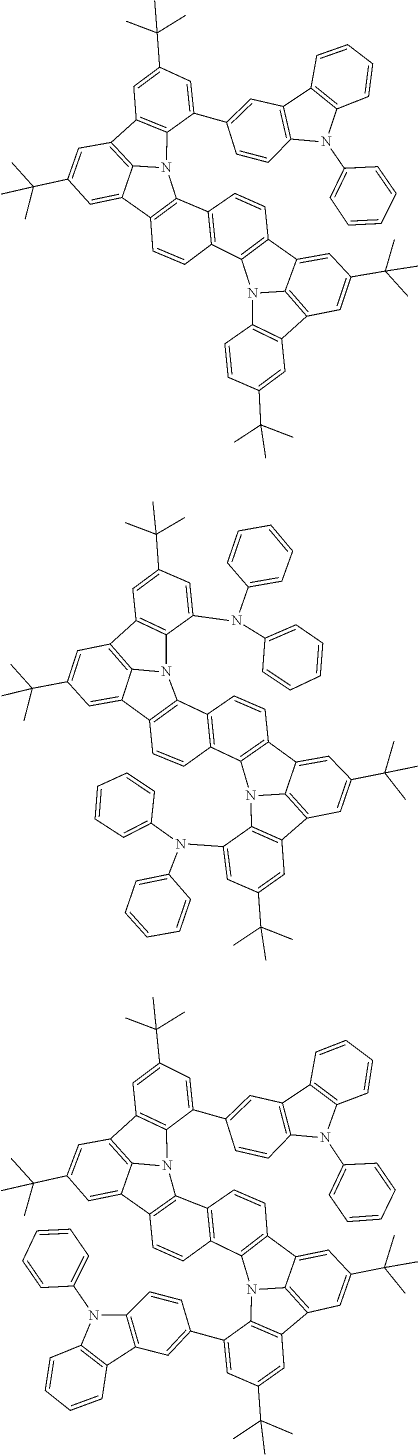

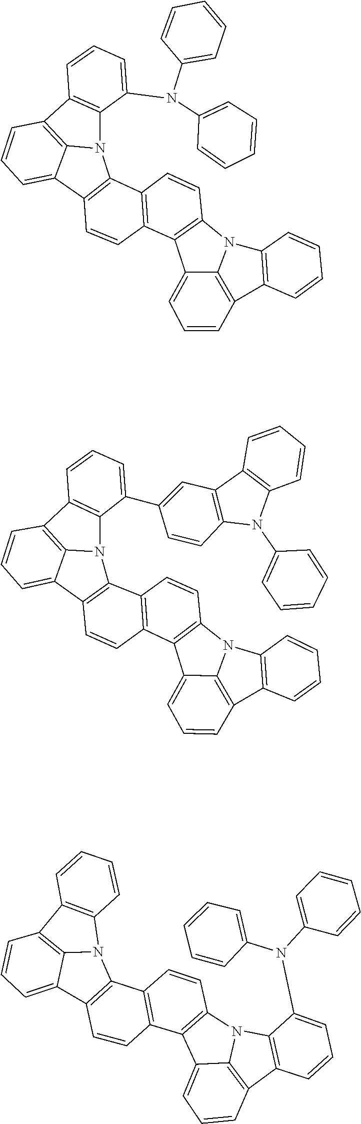 [SMILES: c1ccc(N(c2ccccc2)c2cccc3c4cccc5c6ccc7c(ccc8c7c7cccc9c%10ccccc%10n8c97)c6n(c23)c45)cc1 ; c1ccc(-n2c3ccccc3c3cc(-c4cccc5c6cccc7c8ccc9c(ccc%10c9c9cccc%11c%12ccccc%12n%10c%119)c8n(c45)c67)ccc32)cc1 ; c1ccc(N(c2ccccc2)c2cccc3c4cccc5c6c7ccc8c9cccc%10c%11ccccc%11n(c8c7ccc6n(c23)c45)c%109)cc1]

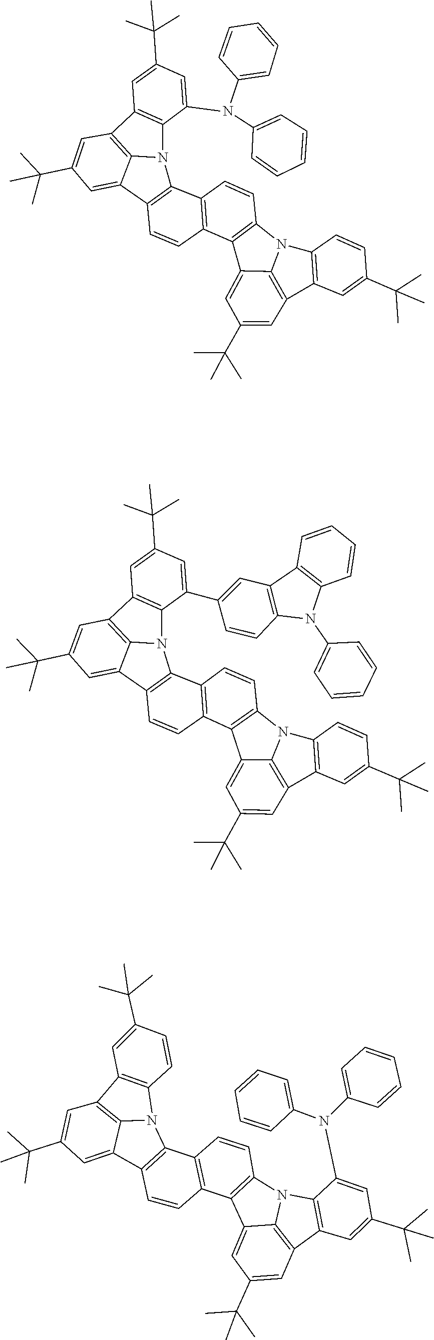

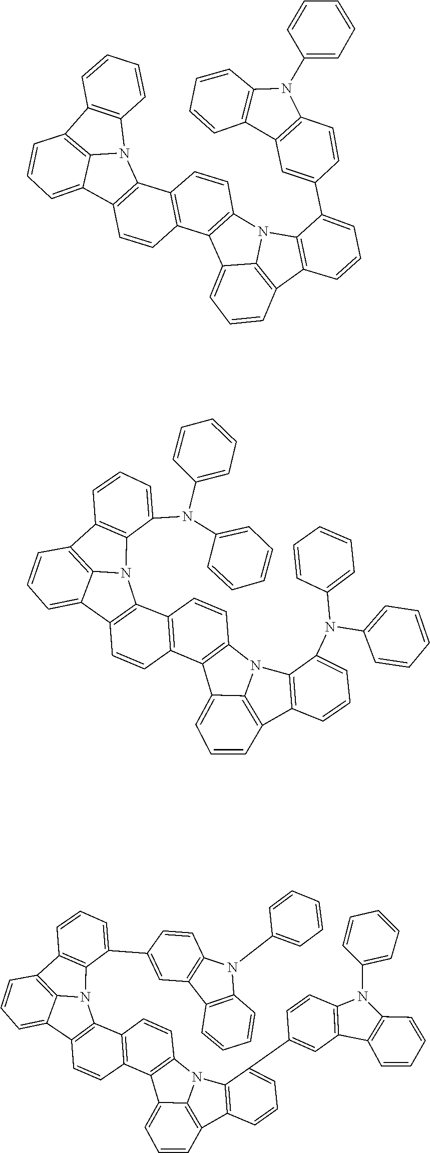 [SMILES: c1ccc(-n2c3ccccc3c3cc(-c4cccc5c6cccc7c8c9ccc%10c%11cccc%12c%13ccccc%13n(c%10c9ccc8n(c45)c67)c%12%11)ccc32)cc1 ; c1ccc(N(c2ccccc2)c2cccc3c4cccc5c6c7ccc8c9cccc%10c%11cccc(N(c%12ccccc%12)c%12ccccc%12)c%11n(c8c7ccc6n(c23)c45)c%109)cc1 ; c1ccc(-n2c3ccccc3c3cc(-c4cccc5c6cccc7c8c9ccc%10c%11cccc%12c%13cccc(-c%14ccc%15c(c%14)c%14ccccc%14n%15-c%14ccccc%14)c%13n(c%10c9ccc8n(c45)c67)c%12%11)ccc32)cc1]

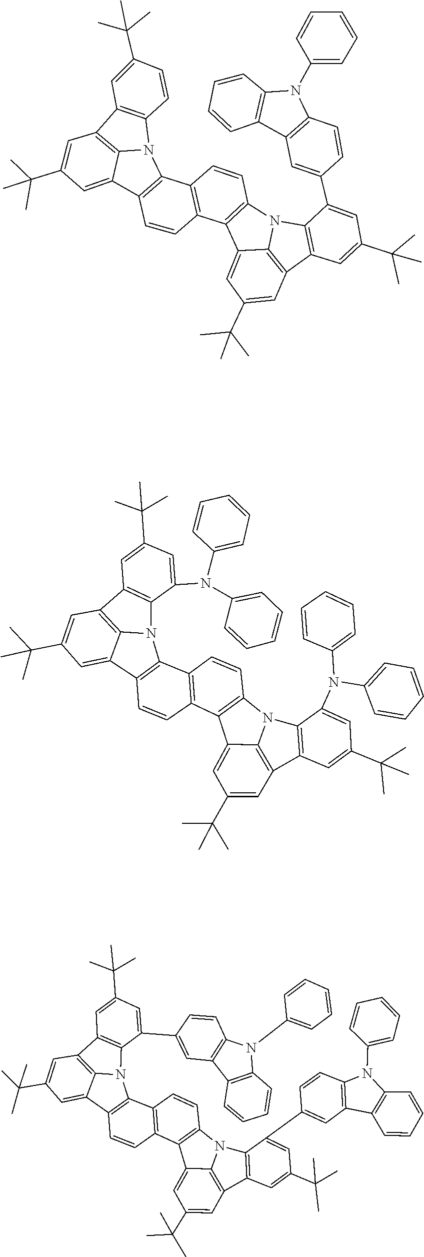

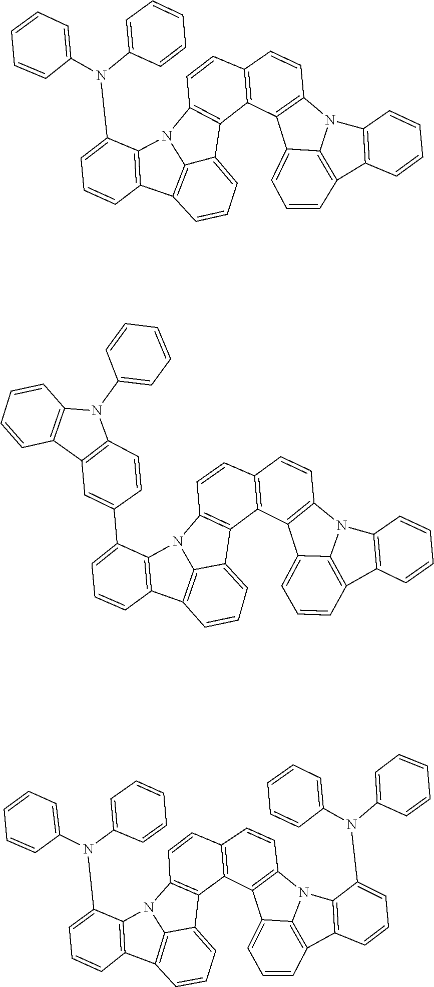 [SMILES: c1ccc(N(c2ccccc2)c2cccc3c4cccc5c6c7c(ccc8c7c7cccc9c%10ccccc%10n8c97)ccc6n(c23)c45)cc1 ; c1ccc(-n2c3ccccc3c3cc(-c4cccc5c6cccc7c8c9c(ccc%10c9c9cccc%11c%12ccccc%12n%10c%119)ccc8n(c45)c67)ccc32)cc1 ; c1ccc(N(c2ccccc2)c2cccc3c4cccc5c6c7c(ccc6n(c23)c45)ccc2c7c3cccc4c5cccc(N(c6ccccc6)c6ccccc6)c5n2c43)cc1]

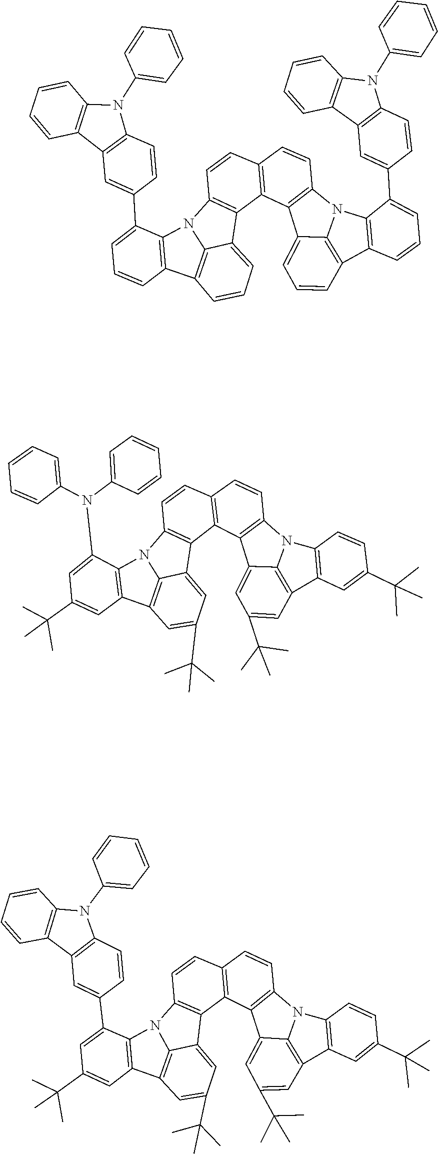 [SMILES: c1ccc(-n2c3ccccc3c3cc(-c4cccc5c6cccc7c8c9c(ccc8n(c45)c67)ccc4c9c5cccc6c7cccc(-c8ccc9c(c8)c8ccccc8n9-c8ccccc8)c7n4c65)ccc32)cc1 ; CC(C)(C)c1ccc2c(c1)c1cc(C(C)(C)C)cc3c4c5c(ccc4n2c13)ccc1c5c2cc(C(C)(C)C)cc3c4cc(C(C)(C)C)cc(N(c5ccccc5)c5ccccc5)c4n1c32 ; CC(C)(C)c1ccc2c(c1)c1cc(C(C)(C)C)cc3c4c5c(ccc4n2c13)ccc1c5c2cc(C(C)(C)C)cc3c4cc(C(C)(C)C)cc(-c5ccc6c(c5)c5ccccc5n6-c5ccccc5)c4n1c32]

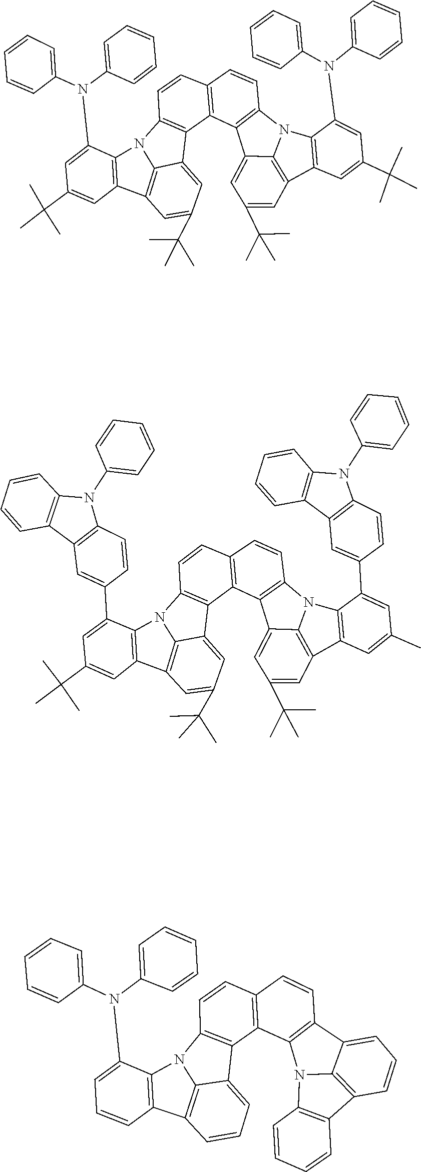 [SMILES: CC(C)(C)c1cc(N(c2ccccc2)c2ccccc2)c2c(c1)c1cc(C(C)(C)C)cc3c4c5c(ccc4n2c13)ccc1c5c2cc(C(C)(C)C)cc3c4cc(C(C)(C)C)cc(N(c5ccccc5)c5ccccc5)c4n1c32 ; Cc1cc(-c2ccc3c(c2)c2ccccc2n3-c2ccccc2)c2c(c1)c1cc(C(C)(C)C)cc3c4c5c(ccc4n2c13)ccc1c5c2cc(C(C)(C)C)cc3c4cc(C(C)(C)C)cc(-c5ccc6c(c5)c5ccccc5n6-c5ccccc5)c4n1c32 ; c1ccc(N(c2ccccc2)c2cccc3c4cccc5c6c7c(ccc8c9cccc%10c%11ccccc%11n(c%109)c87)ccc6n(c23)c45)cc1]

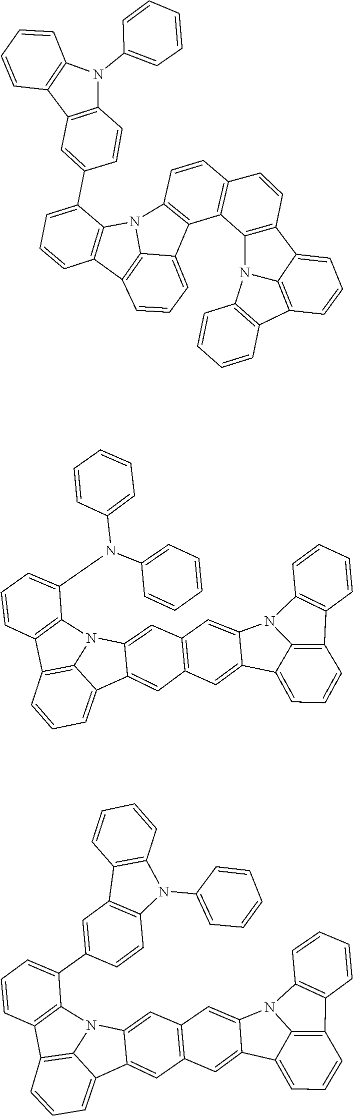 [SMILES: c1ccc(-n2c3ccccc3c3cc(-c4cccc5c6cccc7c8c9c(ccc%10c%11cccc%12c%13ccccc%13n(c%12%11)c%109)ccc8n(c45)c67)ccc32)cc1 ; c1ccc(N(c2ccccc2)c2cccc3c4cccc5c6cc7cc8c9cccc%10c%11ccccc%11n(c8cc7cc6n(c23)c54)c%109)cc1 ; c1ccc(-n2c3ccccc3c3cc(-c4cccc5c6cccc7c8cc9cc%10c%11cccc%12c%13ccccc%13n(c%10cc9cc8n(c45)c76)c%12%11)ccc32)cc1]

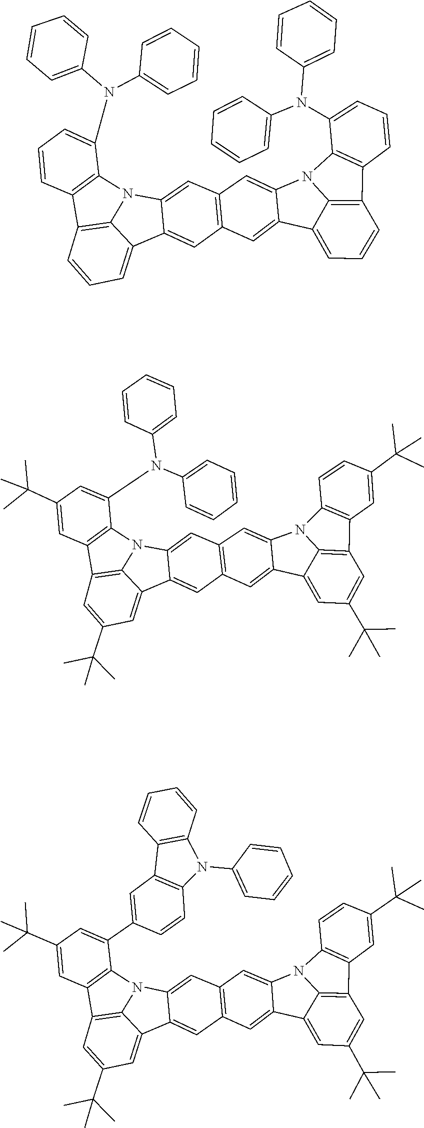

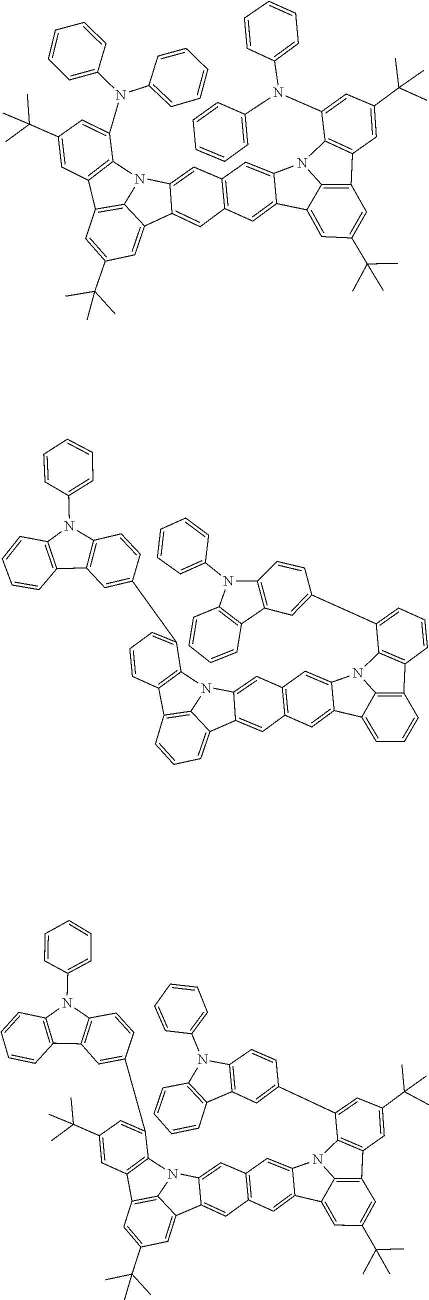 [SMILES: CC(C)(C)c1cc(N(c2ccccc2)c2ccccc2)c2c(c1)c1cc(C(C)(C)C)cc3c4cc5cc6c7cc(C(C)(C)C)cc8c9cc(C(C)(C)C)cc(N(c%10ccccc%10)c%10ccccc%10)c9n(c6cc5cc4n2c31)c78 ; c1ccc(-n2c3ccccc3c3cc(-c4cccc5c6cccc7c8cc9cc%10c%11cccc%12c%13cccc(-c%14ccc%15c(c%14)c%14ccccc%14n%15-c%14ccccc%14)c%13n(c%10cc9cc8n(c45)c76)c%11%12)ccc32)cc1 ; CC(C)(C)c1cc(-c2ccc3c(c2)c2ccccc2n3-c2ccccc2)c2c(c1)c1cc(C(C)(C)C)cc3c4cc5cc6c7cc(C(C)(C)C)cc8c9cc(C(C)(C)C)cc(-c%10ccc%11c(c%10)c%10ccccc%10n%11-c%10ccccc%10)c9n(c6cc5cc4n2c31)c78]

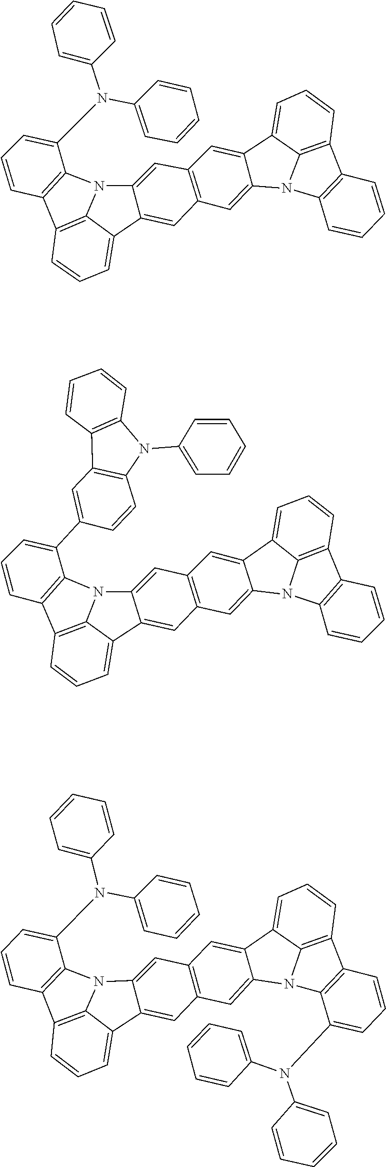 [SMILES: c1ccc(N(c2ccccc2)c2cccc3c4cccc5c6cc7cc8c(cc7cc6n(c23)c54)c2cccc3c4ccccc4n8c32)cc1 ; c1ccc(-n2c3ccccc3c3cc(-c4cccc5c6cccc7c8cc9cc%10c(cc9cc8n(c45)c76)c4cccc5c6ccccc6n%10c54)ccc32)cc1 ; c1ccc(N(c2ccccc2)c2cccc3c4cccc5c6cc7cc8c(cc7cc6n(c23)c54)c2cccc3c4cccc(N(c5ccccc5)c5ccccc5)c4n8c23)cc1]

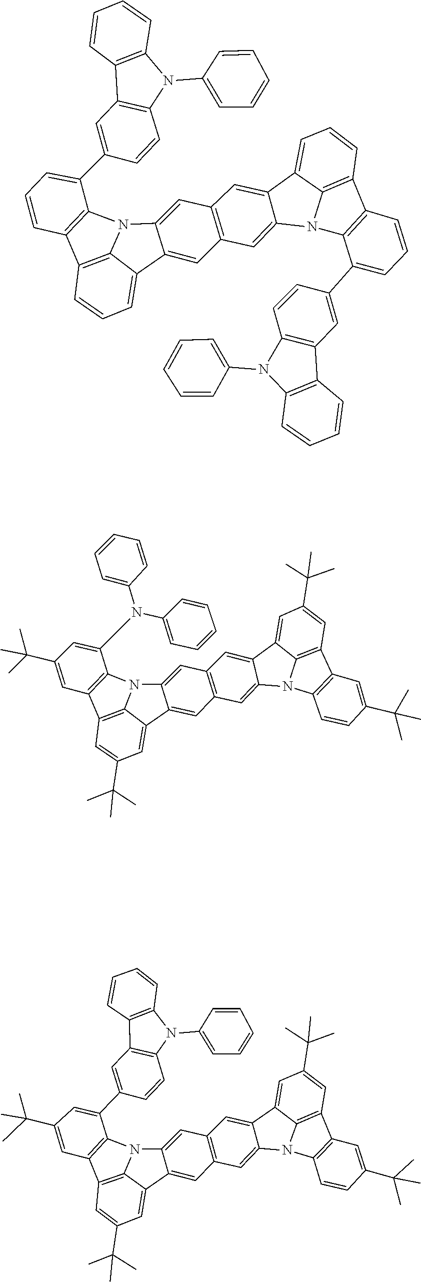

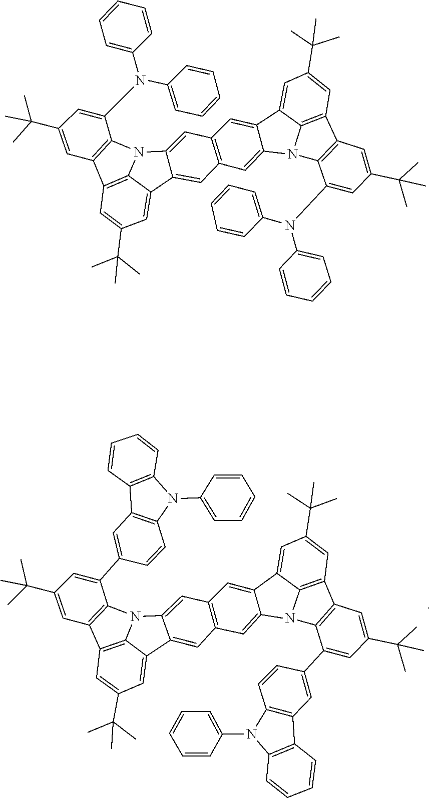

Since the heterocyclic compound has a rigid structure in which aromatic hydrocarbon rings or heteroaromatic rings are condensed, structural relaxation in an excited state may be suppressed. As a result, the heterocyclic compound may have a narrow width of blue emission spectrum and improved colorimetric purity.

The heterocyclic compound may include two partial structures represented by

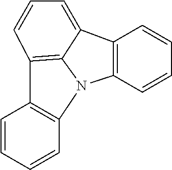 [SMILES: c1ccc2c(c1)c1cccc3c4ccccc4n2c13]

(hereinafter referred to as “ICz partial structure”). In this regard, compared to the heterocyclic compound including one ICz partial structure, the heterocyclic compound disclosed herein may have improved multi-resonance characteristics. Accordingly, regardless of ΔE_{ST} value, the reverse intersystem crossing (RISC) speed may be improved, thereby improving the efficiency of the organic light-emitting device including the heterocyclic compound.

Furthermore, due to the improved multi-resonance characteristics, the heterocyclic compound may have relatively small full width at half maximum (FWHM), and accordingly, the organic light-emitting device including the heterocyclic compound may have improved color purity and/or improved conversion efficiency. For example, the FWHM of the heterocyclic compound may be less than 35 nm.

When the heterocyclic compound serves as a dopant in the organic light-emitting device, the heterocyclic compound may emit blue light, for example, blue light having a maximum emission wavelength of less than or equal to about 550 nm. For example, the maximum emission wavelength of the blue light may be in a range of about 400 nm to about 500 nm, for example, about 420 nm to about 480 nm, or for example, may be less than or equal to about 465 nm. The “maximum emission wavelength” as used herein refers to a wavelength of which the emission intensity is greatest. In other words, the “maximum emission wavelength” may be referred to as “peak emission wavelength.”

When the heterocyclic compound serves as a dopant in the organic light-emitting device, the organic light-emitting device may have CIEy of less than or equal to about 0.09. For example, the CIEy of the organic light-emitting device may be less than or equal to about 0.07.

A peak wavelength at photoluminescence (PL) and FWHM at PL may each be measured and/or calculated using a spectrofluorophotometer.

In the heterocyclic compound, Rₓ is necessarily substituted at a specific position, and thus N of the heterocyclic compound is not exposed to the outside, thereby improving stability of the heterocyclic compound as we all color reproducibility of the heterocyclic compound. In addition, in the heterocyclic compound, Rₓ is necessarily substituted at a specific position, so that steric hindrance of the heterocyclic compound may be improved, thereby reducing interaction with a host. Accordingly, the organic light-emitting device including the heterocyclic compound may provide improved efficiency and improved color reproducibility.

In particular, in the heterocyclic compound, Rₓ is selected as a chromophore substituent having a specific structure including, for example, an amino group or a carbazole, and thus the substitution effect of Rₓ may maximize the luminescence efficiency.

The heterocyclic compound may satisfy Conditions 1 to 4:

|ΔE_{ST}−ΔE′_{TT}|<0.3 eV Condition 1:

0 eV<ΔE_{ST2}+ΔE′_{TT}≤1.0 eV Condition 2:

0 eV<ΔE′_{TT}≤0.30 eV Condition 3:

ΔE_{ST2}>0 eV Condition 4:

In Conditions 1 to 4,
ΔE_{ST} indicates a difference between a lowest excited singlet energy level calculated in an Si equilibrium structure of the heterocyclic compound and a lowest excited triplet energy level calculated in a T₁ equilibrium structure of the heterocyclic compound,
ΔE_{ST2} indicates a difference between a lowest excited singlet energy level calculated in an Si equilibrium structure of the heterocyclic compound and a second lowest excited triplet energy level calculated in a T₂ equilibrium structure of the heterocyclic compound, and
ΔE′_{TT} indicates a difference between a second lowest excited triplet energy level calculated in an T₂ equilibrium structure of the heterocyclic compound and a lowest excited triplet energy level calculated in a T₂ equilibrium structure of the heterocyclic compound.

The equilibrium structure is optimized using a Turbomole program (see [F. Furche et al. WIRESs: Comput. Mol. Sci. 4, 91-100 (2014)]). In detail, a time-dependent density functional theory (DFT) using PBEO functional within the Tamm-Dancoff approximation is used for structural optimization in the T₁, T₂, and S₁ states. Frequency calculations are performed to obtain normal modes, and the lowest energy structure is identified. The nonadiabatic coupling between the excited triplet state and the Ti state is calculated using a Q-Chem program (see [Y. Shao et al. Mol. Phys. 113, 184-215 (2015)]). In addition, the Q-Chem program calculates spin-orbit couplings for TDDFT states using the one-electron Breit-Pauli spin-orbit operator. For all atoms, the def2-SVP basis sets are used.

In detail, the heterocyclic compound may satisfy Condition 3A:

0 eV<ΔE′_{TT}≤0.15 eV Condition 3A:

In Condition 3A, ΔE′TT is the same as described above.

In general, compounds having a relatively small ΔE_{ST} value may emit thermal activated delayed fluorescence (TADF). However, even though the ΔE_{ST} value of the heterocyclic compound is relatively large, as the heterocyclic compound satisfies Conditions 1 to 4, the heterocyclic compound may emit TADF, and an organic light-emitting device including the heterocyclic compound may have improved efficiency.

Furthermore, by using the heterocyclic compound as a sensitizer, energy transferred to a triplet state may undergo RISC to a singlet state. Then, the singlet energy of the heterocyclic compound may be transferred to a dopant by Förster energy transfer. Thus, an organic light-emitting device including the heterocyclic compound may also have improved efficiency and improved lifespan at the same time.

The synthesis method of the heterocyclic compound according to one or more embodiments is not particularly limited and may be synthesized according to a known synthesis method. In particular, it may be synthesized according to or in view of the method described in the Examples. For example, in the method described in the Examples, the heterocyclic compound according to one or more embodiments may be synthesized through modifications such as changing raw materials and reaction conditions, adding or excluding some processes, or appropriately combining with other known synthesis methods.

The method of identifying a structure of the heterocyclic compound according to one or more embodiments is not particularly limited. The heterocyclic compound containing nitrogen according to one or more embodiments may be identified by a known method, for example, NMR or LC-MS.

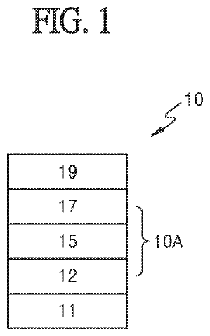

Description of FIG. 1

FIG. 1 is a schematic view of an organic light-emitting device 10 according to an exemplary embodiment. Hereinafter a structure and a method of manufacturing the organic light-emitting device 10, according to an embodiment, will be described with reference to FIG. 1.

In FIG. 1, an organic light-emitting device 10 includes a first electrode 11, a second electrode 19 facing the first electrode 11, and an organic layer 10A between the first electrode 11 and the second electrode 19.

In FIG. 1, the organic layer 10A includes an emission layer 15, a hole transport region 12 is between the first electrode 11 and an emission layer 15, and an electron transport region 17 is between the emission layer 15 and the second electrode 19.

A substrate may be additionally disposed under the first electrode 11 or on the second electrode 19. The substrate may be a conventional substrate used in organic light-emitting devices, e.g., a glass substrate or a transparent plastic substrate, each having excellent mechanical strength, thermal stability, transparency, surface smoothness, ease of handling, and water repellency.

First Electrode 11

The first electrode 11 may be produced by depositing or sputtering, onto the substrate, a material for forming the first electrode 11. The first electrode 11 may be an anode. The material for forming the first electrode 11 may be a material with a high work function for easy hole injection.

The first electrode 11 may be a reflective electrode, a semi-transmissive electrode, or a transmissive electrode. When the first electrode 11 is a transmissive electrode, a material for forming the first electrode 11 may be indium tin oxide (ITO), indium zinc oxide (IZO), tin oxide (SnO₂), zinc oxide (ZnO), and any combinations thereof, but embodiments are not limited thereto. In one or more embodiments, when the first electrode 11 is a semi-transmissive electrode or a reflective electrode, a material for forming the first electrode 11 may be magnesium (Mg), silver (Ag), aluminum (Al), aluminum-lithium (Al—Li), calcium (Ca), magnesium-indium (Mg—In), magnesium-silver (Mg—Ag), and any combination thereof, but embodiments of the present disclosure are not limited thereto.

The first electrode 11 may have a single-layer structure or a multi-layer structure including multiple layers.

Emission Layer 15

The emission layer 15 may include the heterocyclic compound.

A thickness of the emission layer may be in a range of about 100 Å to about 1,000 Å, for example, about 200 Å to about 600 Å. When the thickness of the emission layer is within these ranges, excellent luminescence characteristics may be obtained without a substantial increase in driving voltage.

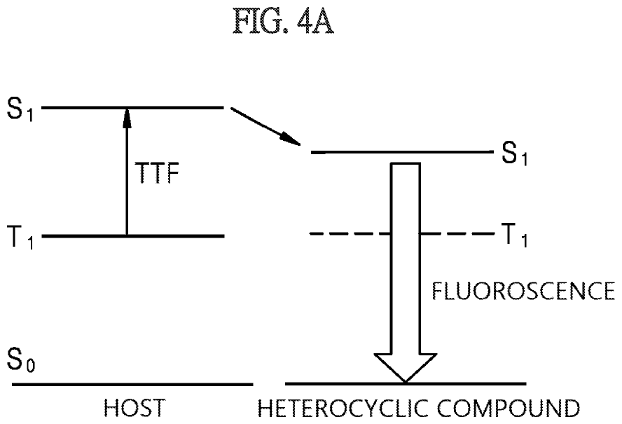

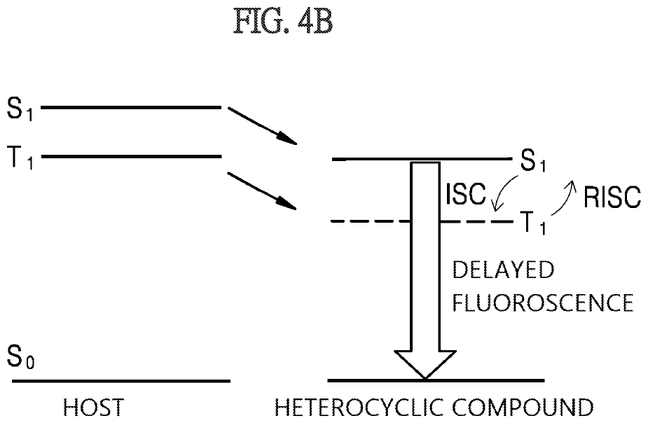

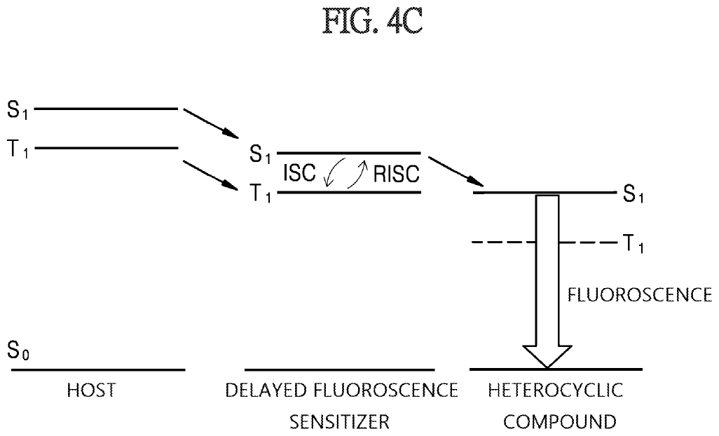

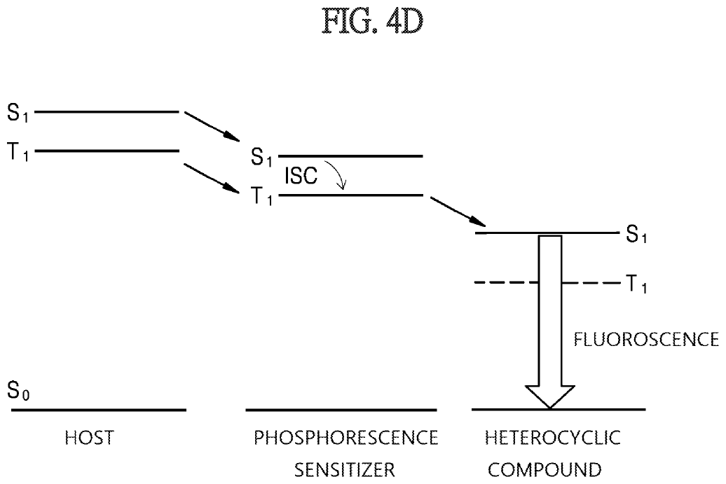

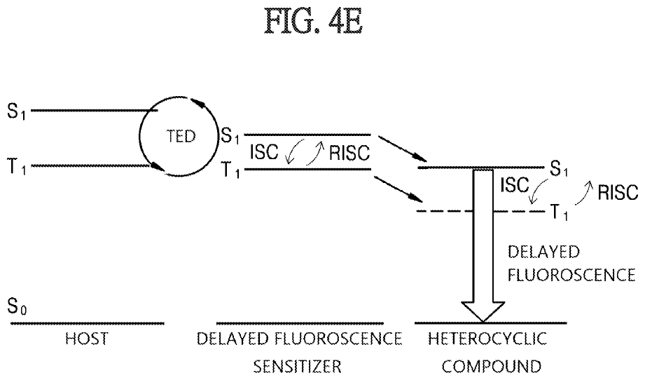

### First Embodiment]—Description of FIG. 4A

In First Embodiment, the heterocyclic compound may be a fluorescence emitter. In First Embodiment, the emission layer may further include a host (hereinafter, referred to as ‘Host A’, and Host A is not identical to the heterocyclic compound). Host A may be understood by referring to the description of a host material below, but embodiments of the present disclosure are not limited thereto. Host A may be a fluorescent host.

General energy transfer in First Embodiment may be explained according to FIG. 4A.

Singlet excitons may be produced from Host A in the emission layer, and singlet excitons produced from Host A may be transferred to a fluorescence emitter through Förster energy transfer (FRET).

A ratio of singlet excitons produced from Host A may be 25%, and thus, 75% of triplet excitons produced from Host A may be fused to one another to be converted into singlet excitons. Thus, the efficiency of the organic light-emitting device may be further improved. That is, the efficiency of an organic light-emitting device may be further improved by using a triplet-triplet fusion mechanism.

In First Embodiment, a ratio of emission components emitted from the heterocyclic compound to the total emission components emitted from the emission layer may be equal to or greater than 80%, for example, equal to or greater than 90%. For example, a ratio of emission components emitted from the heterocyclic compound to the total emission components emitted from the emission layer may be equal to or greater than 95%.

The heterocyclic compound may emit fluorescence, and the host may not emit light.

In First Embodiment, when the emission layer further includes Host A, in addition to the heterocyclic compound, a content of the heterocyclic compound may be, based on 100 parts by weight of the emission layer, less than or equal to about 50 parts by weight, for example, less than or equal to about 30 parts by weight or less, and a content of Host A in the emission layer may be, based on 100 parts by weight of the emission layer, equal to or greater than about 50 parts by weight, for example, equal to or greater than about 70 parts by weight. However, embodiments of the present disclosure are not limited thereto.

In First Embodiment, when the emission layer further includes Host A, in addition to the heterocyclic compound, Host A and the heterocyclic compound may satisfy Condition A.

E(H_{A})_{S1}>E_{S1} Condition A:

In Condition A,
E(H_{A})_{S1} indicates a lowest excited singlet energy level of Host A, and
E_{S1} indicates a lowest excited singlet energy level of the heterocyclic compound.

In Condition A, E(H_{A})_{S1} and E_{S1} may be evaluated by using a Gaussian according to the DFT method, wherein structure optimization is performed at a degree of B3LYP, and 6-31G(d,p)).

### Second Embodiment—Description of FIG. 4B

In Second Embodiment, the heterocyclic compound may be a delayed fluorescence emitter. In Second Embodiment, the emission layer may further include a host (hereinafter, referred to as ‘Host B’, and Host B is not identical to the heterocyclic compound). Host B may be understood by referring to the description of a host material below, but embodiments of the present disclosure are not limited thereto.

General energy transfer in Second Embodiment may be explained according to FIG. 4B.

25% of singlet excitons produced from Host B in the emission layer may be transferred to a delayed fluorescence emitter through FRET. In addition, 75% of triplet excitons produced from Host B in the emission layer may be transferred to a delayed fluorescence emitter through Dexter energy transfer. Energy transferred to a triplet state of a delayed fluorescence emitter may undergo RISC to a singlet state. Accordingly, by transferring all the singlet excitons and triplet excitons generated in the emission layer to the heterocyclic compound, an organic light-emitting device including the heterocyclic compound and having improved efficiency may be obtained.

In Second Embodiment, a ratio of emission components emitted from the heterocyclic compound to the total emission components emitted from the emission layer may be equal to or greater than about 80%, for example, equal to or greater than about 90%. For example, a ratio of emission components emitted from the heterocyclic compound to the total emission components emitted from the emission layer may be equal to or greater than about 95%.

Here, the heterocyclic compound may emit fluorescence and/or delayed fluorescence, and the emission components of the heterocyclic compound may be a total of prompt emission components of the heterocyclic compound and delayed fluorescence components by RISC of the heterocyclic compound. In addition, Host B may not emit light.

In Second Embodiment, when the emission layer further includes Host B, in addition to the heterocyclic compound, a content of the heterocyclic compound may be, based on 100 parts by weight of the emission layer, less than or equal to about 50 parts by weight, for example, less than or equal to about 30 parts by weight or less, and a content of Host B in the emission layer may be, based on 100 parts by weight of the emission layer, equal to or greater than about 50 parts by weight, for example, equal to or greater than about 70 parts by weight. However, embodiments of the present disclosure are not limited thereto.

In Second Embodiment, when the emission layer further includes Host B, in addition to the heterocyclic compound, Host B and the heterocyclic compound may satisfy Condition B.

E(H_{B})_{S1}>E_{S1} Condition B:

In Condition B,
E(H_{B})_{S1} indicates a lowest excited singlet energy level of Host B, and
E_{S1} indicates a lowest excited singlet energy level of the heterocyclic compound.

In Condition B, E(H_{A})_{S1} and E_{S1} may be evaluated by using a Gaussian according to the DFT method, wherein structure optimization is performed at a degree of B3LYP, and 6-31G(d,p)).

### Third Embodiment and Fourth Embodiment

### Third Embodiment—Description of FIG. 4C

In Third Embodiment, the heterocyclic compound may be used as a fluorescence emitter, and the emission layer may include a sensitizer, e.g., a delayed fluorescence sensitizer. In Third Embodiment, the emission layer may further include a host (hereinafter, the host may be referred to as ‘Host C’, and Host C is not identical to the heterocyclic compound and the sensitizer) and a sensitizer (hereinafter, the sensitizer may be referred to as ‘Sensitizer A’, and Sensitizer A is not identical to Host C and the heterocyclic compound). Host C and Sensitizer A may respectively be understood by referring to the description of a host material and a sensitizer material below, but embodiments of the present disclosure are not limited thereto.

In Third Embodiment, a ratio of emission components of the heterocyclic compound to the total emission components emitted from the emission layer may be equal to or greater than about 80%, for example, equal to or greater than about 90% (or for example, equal to or greater than about 95%). For example, the heterocyclic compound may emit fluorescence. In addition, Host C and Sensitizer A may not each emit light.

General energy transfer in Third Embodiment may be explained according to FIG. 4C.

Singlet and triplet excitons may be produced from Host C in the emission layer, and singlet and triplet excitons produced from Host C may be transferred to Sensitizer A and then to the heterocyclic compound through FRET. 25% of singlet excitons produced from Host C may be transferred to Sensitizer A through FRET, and energy of 75% of triplet excitons produced from Host C may be transferred to singlet and triplet states of Sensitizer A. Energy transferred to a triplet state of Sensitizer A may undergo RISC to a singlet state, and then, singlet energy of Sensitizer A may be transferred to the heterocyclic compound through FRET.

Accordingly, by transferring all the singlet excitons and triplet excitons generated in the emission layer to the dopant, an organic light-emitting device having improved efficiency may be obtained. In addition, since an organic light-emitting device may be obtained with significantly reduced energy loss, the lifespan characteristics of the organic light-emitting device may be also improved.

In Third Embodiment, when the emission layer further includes Host C and Sensitizer A, in addition to the heterocyclic compound, Host C and Sensitizer A may satisfy Condition C-1 and/or C-2.

S₁(H_{C})≥S₁(S_{A}) Condition C-1:

S₁(S_{A})≥S₁(HC) Condition C-2:

In Conditions C-1 and C-2,
S₁(H_{C}) indicates a lowest excited singlet energy level of Host C,
S₁(S_{A}) indicates a lowest excited singlet energy level of Sensitizer A, and
S₁(HC) indicates a lowest excited singlet energy level of the heterocyclic compound.

S₁(H_{C}), S₁(S_{A}), and S₁(HC) may be evaluated by using a Gaussian according to the DFT method, wherein structure optimization is performed at a degree of B3LYP, and 6-31G(d,p)).

When Host C, Sensitizer A, and the heterocyclic compound satisfy Condition C-1 and/or C-2, FRET from Sensitizer A to the heterocyclic compound may be facilitated, and accordingly, the organic light-emitting device may have improved luminescence efficiency.

### Fourth Embodiment—Description of FIG. 4D

In Fourth Embodiment, the heterocyclic compound may be used as a fluorescence emitter, and the emission layer may include a sensitizer, e.g., a phosphorescence sensitizer. In Fourth Embodiment, the emission layer may further include a host (hereinafter, the host may be referred to as ‘Host D’, and Host D is not identical to the heterocyclic compound and the sensitizer) and a sensitizer (hereinafter, the sensitizer may be referred to as ‘Sensitizer B’, and Sensitizer B is not identical to Host D and the heterocyclic compound). Host D and Sensitizer B may respectively be understood by referring to the description of a host material and a sensitizer material below, but embodiments of the present disclosure are not limited thereto.

In Fourth Embodiment, a ratio of emission components of the heterocyclic compound to the total emission components emitted from the emission layer may be equal to or greater than about 80%, for example, equal to or greater than about 90% (or for example, equal to or greater than about 95%). For example, the heterocyclic compound may emit fluorescence. In addition, Host D and Sensitizer B may not each emit light.

General energy transfer in Fourth Embodiment may be explained according to FIG. 4D.

75% of triplet excitons produced from Host D in the emission layer may be transferred to Sensitizer B through Dexter energy transfer, and energy of 25% of singlet excitons produced from Host D may be transferred to singlet and triplet states of Sensitizer B. Energy transferred to a singlet state of Sensitizer B may undergo ISC to a triplet state, and then, triplet energy of Sensitizer B may be transferred to the heterocyclic compound through FRET.

Accordingly, by transferring all the singlet excitons and triplet excitons generated in the emission layer to the dopant, an organic light-emitting device having improved efficiency may be obtained. In addition, since an organic light-emitting device may be obtained with significantly reduced energy loss, the lifespan characteristics of the organic light-emitting device may be also improved.

In Fourth Embodiment, when the emission layer further includes Host D and Sensitizer B, in addition to the heterocyclic compound, Host D and Sensitizer B may satisfy Condition D-1 and/or D-2.

T₁(H_{D})≥T₁(S_{B}) Condition D-1:

T₁(S_{B})≥S₁(HC) Condition D-2:

In Conditions D-1 and D-2,
T₁(H_{D}) indicates a lowest excited triplet energy level of Host D,
T₁(S_{B}) indicates a lowest excited triplet energy level of Sensitizer B, and
S₁(HC) indicates a lowest excited singlet energy level of the heterocyclic compound.

T₁(Ho), T₁(S_{B}), and S₁(HC) may be evaluated by using a Gaussian according to the DFT method, wherein structure optimization is performed at a degree of B3LYP, and 6-31G(d,p)).

When Host D, Sensitizer B, and the heterocyclic compound satisfy Condition D-1 and/or D-2, FRET from Sensitizer B to the heterocyclic compound may be facilitated, and accordingly, an organic light-emitting device including the heterocyclic compound may have improved luminescence efficiency.

In Third Embodiment and Fourth Embodiment, a content of the sensitizer in the emission layer may be in a range of about 5 wt % to about 50 wt %, or for example, about 10 wt % to about 30 wt %. When the content is within these ranges, effective energy transfer in the emission layer may be achieved. Thus, an organic light-emitting device including the heterocyclic compound may have high efficiency and a long lifespan.

In Third Embodiment and Fourth Embodiment, a content of the heterocyclic compound in the emission layer may be in a range of about 0.01 wt % to about 15 wt %, for example, about 0.05 wt % to about 3 wt %, but embodiments of the present disclosure are not limited thereto.

In Third Embodiment and Fourth Embodiment, the sensitizer and the heterocyclic compound may further satisfy Condition 5.

0 μs<T_{decay}(HC)<5 μs Condition 5:

In Condition 5, T_{decay}(HC) indicates a decay time of the heterocyclic compound.

The decay time of the heterocyclic compound was measured from a time-resolved photoluminescence (TRPL) spectrum at room temperature of a film (hereinafter, referred to as “Film (HC)”) having a thickness of 40 nm formed by vacuum-depositing the host and the heterocyclic compound included in the emission layer on a quartz substrate at a weight ratio of 90:10 at a vacuum pressure of 10-7 torr.

### Fifth Embodiment—Description of FIG. 4E

In Fifth Embodiment, the heterocyclic compound may be used as a delayed fluorescence emitter, and the emission layer may include a sensitizer, e.g., a delayed fluorescence sensitizer. In Fifth Embodiment, the emission layer may further include a host (hereinafter, the host may be referred to as ‘Host E’, and Host E is not identical to the heterocyclic compound and the sensitizer) and a sensitizer (hereinafter, the sensitizer may be referred to as ‘Sensitizer C’, and Sensitizer C is not identical to Host E and the heterocyclic compound). Host E and Sensitizer C may respectively be understood by referring to the description of a host material and a sensitizer material below, but embodiments of the present disclosure are not limited thereto.

In Fifth Embodiment, a ratio of emission components of the heterocyclic compound to the total emission components emitted from the emission layer may be equal to or greater than about 80%, for example, equal to or greater than about % (or for example, equal to or greater than about 95%). For example, the heterocyclic compound may emit fluorescence and/or delayed fluorescence. In addition, Host E and Sensitizer C may not each emit light.

Here, the heterocyclic compound may emit fluorescence and/or delayed fluorescence, and the emission components of the heterocyclic compound may be a total of prompt emission components of the heterocyclic compound and delayed fluorescence components by RISC of the heterocyclic compound.

General energy transfer in Fifth Embodiment may be explained according to FIG. 4E.

25% of singlet excitons produced from Host E in the emission layer may be transferred to a singlet state of Sensitizer C through FRET, and energy of 75% of triplet excitons produced from Host E may be transferred to a triplet state of Sensitizer C, and then singlet energy of Sensitizer C may be transferred to the heterocyclic compound through FRET. Subsequently, the triplet energy of Sensitizer C may be transferred to the heterocyclic compound through Dexter energy transfer. Energy transferred to a triplet state of Sensitizer C may undergo RISC to a singlet state. Further, in a case of Sensitizer C, energy of triplet excitons produced from Sensitizer C may undergo reverse transfer to Host E and then to the heterocyclic compound, thus emitting by reverse intersystem transfer.

Accordingly, by transferring all the singlet excitons and triplet excitons generated in the emission layer to the dopant, an organic light-emitting device having improved efficiency may be obtained. In addition, since an organic light-emitting device may be obtained with significantly reduced energy loss, the lifespan characteristics of the organic light-emitting device may be also improved.

In Fifth Embodiment, when the emission layer further includes Host E and Sensitizer C, in addition to the heterocyclic compound, Host E and Sensitizer C may satisfy Condition E-1, E-2, and/or E-3.

S₁(H_{E})≥S₁(S_{C}) Condition E-1:

S₁(S_{C})≥S₁(HC) Condition E-2:

T₁(S_{C})≥T₁(HC) Condition E-3:

In Conditions E-1, E-2, and E-3,
S₁(H_{E}) indicates a lowest excited singlet energy level of Host E,
S₁(S_{C}) indicates a lowest excited singlet energy level of Sensitizer C,
S₁(HC) indicates the lowest excited singlet energy level of the heterocyclic compound,
T₁(S_{C}) indicates a lowest excited triplet energy level of Sensitizer C, and
T₁(HC) indicates a lowest excited triplet energy level of the heterocyclic compound.
S1(HE), S₁(S_{C}), S₁(HC), T₁(S_{C}), and T₁(HC may be evaluated by using a Gaussian according to the DFT method, wherein structure optimization is performed at a degree of B3LYP, and 6-31G(d,p)).

When Host E, Sensitizer C, and the heterocyclic compound satisfy Condition E-1, E-2, and/or E-3, Dexter transfer FRET from Sensitizer C to the heterocyclic compound may be facilitated, and accordingly, tan organic light-emitting device including the heterocyclic compound may have improved luminescence efficiency.

In Fifth Embodiment, a content of Sensitizer C in the emission layer may be in a range of about 5 wt % to about 50 wt %, or for example, about 10 wt % to about 30 wt %. When the content is within these ranges, effective energy transfer in the emission layer may be achieved. Thus, an organic light-emitting device including the heterocyclic compound may have high efficiency and a long lifespan.

In Fifth Embodiment, a content of the heterocyclic compound in the emission layer may be in a range of about 0.01 wt % to about 15 wt %, or for example, about 0.05 wt % to about 3 wt %, but embodiments of the present disclosure are not limited thereto.

Host in Emission Layer 15

The host may not include a metal atom.

In an embodiment, the host may include one kind of host. When the host includes one kind of host, the one kind of host may be a bipolar host, an electron-transporting host, or a hole-transporting host, which will be described later.

In one or more embodiments, the host may include a mixture of two or more different hosts. For example, the host may be a mixture of an electron-transporting host and a hole-transporting host, a mixture of two types of electron-transporting hosts different from each other, or a mixture of two types of hole-transporting hosts different from each other. The electron-transporting host and the hole-transporting host may be understood by referring to the related description to be presented later.

In one or more embodiments, the host may include an electron-transporting host including at least one electron-transporting moiety and a hole-transporting host that is free of an electron-transporting moiety.

The electron-transporting moiety used herein may be a cyano group, a π electron-deficient nitrogen-containing cyclic group, and a group represented by one of the following formulae:

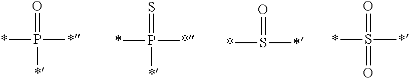

wherein, in the formulae above, *, *′, and *″ each indicate a binding site to a neighboring atom.

In an embodiment, the electron-transporting host in the emission layer 15 may include at least one of a cyano group, a π electron-deficient nitrogen-containing cyclic group, or a combination thereof.

In one or more embodiments, the electron-transporting host in the emission layer 15 may include at least one cyano group.

In one or more embodiments, the electron-transporting host in the emission layer 15 may include at least one cyano group, at least one π electron deficient nitrogen-containing cyclic group, or a combination thereof.

In one or more embodiments, the host may include an electron-transporting host and a hole-transporting host, wherein the electron-transporting host may include at least one π electron-deficient nitrogen-free cyclic group, at least one electron-transporting moiety, or a combination thereof, and the hole-transporting host may include at least one π electron-deficient nitrogen-free cyclic group and may not include an electron-transporting moiety.

The term “π electron-deficient nitrogen-containing cyclic group” as used herein refers to a cyclic group having at least one *—N═*′ moiety, and for example, may be an imidazole group, a pyrazole group, a thiazole group, an isothiazole group, an oxazole group, an isoxazole group, a pyridine group, a pyrazine group, a pyridazine group, a pyrimidine group, an indazole group, a purine group, a quinoline group, an isoquinoline group, a benzoquinoline group, a phthalazine group, a naphthyridine group, a quinoxaline group, a quinazoline group, a cinnoline group, a phenanthridine group, an acridine group, a phenanthroline group, a phenazine group, a benzimidazole group, an isobenzothiazole group, a benzoxazole group, an isobenzoxazole group, a triazole group, a tetrazole group, an oxadiazole group, a triazine group, a thiadiazole group, an imidazopyridine group, an imidazopyrimidine group, an azacarbazole group; or a condensed cyclic group in which two or more π electron-efficient nitrogen-containing cyclic groups are condensed with each other.

Meanwhile, the π electron-deficient nitrogen-free cyclic group may be a benzene group, a heptalene group, an indene group, a naphthalene group, an azulene group, an indacene group, an acenaphthylene group, a fluorene group, a spiro-bifluorene group, a benzofluorene group, a dibenzofluorene group, a phenalene group, a phenanthrene group, an anthracene group, a fluoranthene group, a triphenylene group, a pyrene group, a chrysene group, a naphthacene group, a picene group, a perylene group, a pentacene group, a hexacene group, a pentacene group, a rubicene group, a coragen group, an ovalene group, a pyrrole group, an isoindole group, an indole group, a furan group, a thiophene group, a benzofuran group, a benzothiophene group, a benzocarbazole group, a dibenzocarbazole group, a dibenzofuran group, a dibenzothiophene group, a dibenzothiophene sulfone group, a carbazole group, a dibenzosilole group, an indenocarbazole group, an indolocarbazole group, a benzofurocarbazole group, a benzothienocarbazole group, a triindolobenzene group, or a condensed cyclic group of two or more π electron-deficient nitrogen-free cyclic groups, but embodiments of the present disclosure are not limited thereto.

In one or more embodiments, the electron-transporting host may be a compound represented by Formula E-1, and
the hole-transporting host may be compounds represented by Formula H-1, but embodiments of the present disclosure are not limited thereto:

[Ar₃₀₁]_{xb11}-[(L₃₀₁)_{xb1}-R₃₀₁]_{xb21} Formula E-1
wherein, in Formula E-1,
Ar₃₀₁ may be a substituted or unsubstituted C₅-C₆₀ carbocyclic group and a substituted or unsubstituted C₁-C₆₀ heterocyclic group,
xb11 may be 1, 2, or 3,
L₃₀₁ may each independently be a single bond, groups represented by one of following formulae, a substituted or unsubstituted C₅-C₆₀ carbocyclic group, and a substituted or unsubstituted C₁-C₆₀ heterocyclic group, wherein in the following formulae, *′, and *″ may each indicate a binding site to an adjacent atom,

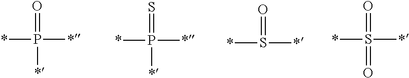

xb1 may be an integer from 1 to 5,
R₃₀₁ may be hydrogen, deuterium, —F, —Cl, —Br, a hydroxyl group, a cyano group, a nitro group, an amidino group, a hydrazino group, a hydrazono group, a substituted or unsubstituted C₁-C₆₀ alkyl group, a substituted or unsubstituted C₂-C₆₀ alkenyl group, a substituted or unsubstituted C₂-C₆₀ alkynyl group, a substituted or unsubstituted C₁-C₆₀ alkoxy group, a substituted or unsubstituted C₃-C₁₀ cycloalkyl group, a substituted or unsubstituted heterocycloalkyl group, a substituted or unsubstituted C₃-C₁₀ cycloalkenyl group, a substituted or unsubstituted C₂-C₁₀ heterocycloalkenyl group, a substituted or unsubstituted C₆-C₆₀ aryl group, a substituted or unsubstituted C₆-C₆₀ aryloxy group, a substituted or unsubstituted C₆-C₆₀ arylthio group, a substituted or unsubstituted C₁-C₆₀ heteroaryl group, a substituted or unsubstituted monovalent aromatic condensed heteropolycyclic group, a substituted or unsubstituted monovalent aromatic condensed heteropolycyclic group, a substituted or unsubstituted monovalent non-aromatic condensed polycyclic group, a substituted or unsubstituted monovalent non-aromatic condensed heteropolycyclic group, —Si(Q₃₀₁)(Q₃₀₂)(Q₃₀₃), —N(Q₃₀₁)(Q₃₀₂), —B(Q₃₀₁)(Q₃₀₂), —C(═O)(Q₃₀₁), —S(═O)₂(Q₃₀₁), —S(═O)(Q₃₀₁), —P(═O)(Q₃₀₁)(Q₃₀₂), or —P(═S)(Q₃₀₁)(Q₃₀₂),
xb21 may be an integer from 1 to 5,
Q₃₀₁ to Q₃₀₃ may each independently be a C₁-C₁₀ alkyl group, a C₁-C₁₀ alkoxy group, a phenyl group, a biphenyl group, a terphenyl group, or a naphthyl group, and
at least one of Condition H-1 to Condition H-3 may be satisfied.

Condition H-1: at least one of Ar₃₀₁, L₃₀₁, and R₃₀₁ in Formula E-1 each independently includes a π electron-depleted nitrogen-containing cyclic group;

Condition H-2: L₃₀₁ in Formula E-1 is a group represented by one of the following formulae; and

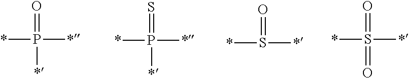

Condition H-3: R₃₀₁ in Formula E-1 is a cyano group, —S(═O)₂(Q₃₀₁), —S(═O)(Q₃₀₁), —P(═O)(Q₃₀₁)(Q₃₀₂), and —P(═S)(Q₃₀₁)(Q₃₀₂),

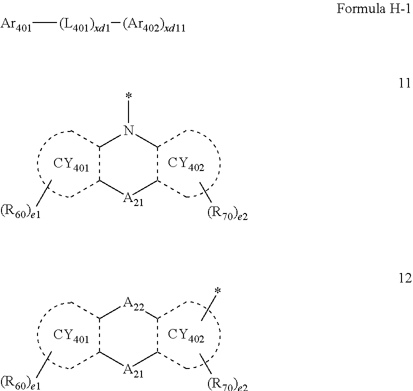

wherein, in Formulae H-1, 11, and 12,
L₄₀₁ may be:
a single bond; or
a benzene group, a heptalene group, an indene group, a naphthalene group, an azulene group, an indacene group, an acenaphthylene group, a fluorene group, a spiro-bifluorene group, a benzofluorene group, a dibenzofluorene group, a phenalene group, a phenanthrene group, an anthracene group, a fluoranthene group, a triphenylene group, a pyrene group, a chrysene group, a naphthacene group, a picene group, a perylene group, a pentacene group, a hexacene group, a pentacene group, a rubicene group, a coragen group, an ovalene group, a pyrrole group, an isoindole group, an indole group, a furan group, a thiophene group, a benzofuran group, a benzothiophene group, a benzocarbazole group, a dibenzocarbazole group, a dibenzofuran group, a dibenzothiophene group, a dibenzothiophene sulfone group, a carbazole group, a dibenzosilole group, an indenocarbazole group, an indolocarbazole group, a benzofurocarbazole group, a benzothienocarbazole group, or a triindolobenzene group, each unsubstituted or substituted with at least one deuterium, a C₁-C₁₀ alkyl group, a C₁-C₁₀ alkoxy group, a phenyl group, a naphthyl group, a fluorenyl group, a carbazolyl group, a dibenzofuranyl group, a dibenzothiophenyl group, a triphenylenyl group, a biphenyl group, a terphenyl group, a tetraphenyl group, —Si(Q₄₀₁)(Q₄₀₂)(Q₄₀₃), or a combination thereof,
xd1 may be an integer from 1 to 10, wherein, when xd1 is 2 or greater, two or more of L₄₀₁ may be identical to or different from each other,
Ar₄₀₁ may be a group represented by Formula 11 or 12,
Ar₄₀₂ may be:
a group represented by Formula 11 or 12, a phenyl group, a naphthyl group, a fluorenyl group, a carbazolyl group, a dibenzofuranyl group, a dibenzothiophenyl group, a biphenyl group, a terphenyl group, or a triphenylenyl group; or
a phenyl group, a naphthyl group, a fluorenyl group, a carbazolyl group, a dibenzofuranyl group, a dibenzothiophenyl group, a biphenyl group, a terphenyl group, or a triphenylenyl group, each substituted with at least one deuterium, a hydroxyl group, an amino group, an amidino group, a hydrazine group, a hydrazone group, a carboxylic acid or a salt thereof, a sulfonic acid or a salt thereof, a phosphoric acid or a salt thereof, a C₁-C₂₀ alkyl group, a C₁-C₂₀ alkoxy group, a phenyl group, a naphthyl group, a fluorenyl group, a carbazolyl group, a dibenzofuranyl group, a dibenzothiophenyl group, a biphenyl group, a terphenyl group, a triphenylenyl group, or a combination thereof,
CY₄₀₁ and CY₄₀₂ may each independently be a benzene group, a naphthalene group, a fluorene group, a carbazole group, a benzocarbazole group, an indolocarbazole group, a dibenzofuran group, a dibenzothiophene group, a dibenzosilole group, a benzonaphthofuran group, a benzonapthothiophene group, or a benzonaphthosilole group,
A₂₁ may be a single bond, O, S, N(R₅₁), C(R₅₁)(R₅₂), or Si(R₅₁)(R₅₂),
A₂₂ may be a single bond, O, S, N(R₅₃), C(R₅₃)(R₅₄), or Si(R₅₃)(R₅₄),
at least one of A₂₁ and A₂₂ in Formula 12 may not be a single bond,
R₅₁ to R₅₄, R₆₀, and R₇₀ may each independently be:
hydrogen, deuterium, a hydroxyl group, an amino group, an amidino group, a hydrazine group, a hydrazone group, a carboxylic acid group or a salt thereof, a sulfonic acid group or a salt thereof, a phosphoric acid group or a salt thereof, a C₁-C₂₀ alkyl group, or a C₁-C₂₀ alkoxy group;
a C₁-C₂₀ alkyl group and a C₁-C₂₀ alkoxy group, each substituted with at least one deuterium, a hydroxyl group, an amino group, an amidino group, a hydrazine group, a hydrazone group, a carboxylic acid group or a salt thereof, a sulfonic acid group or a salt thereof, a phosphoric acid group or a salt thereof, a phenyl group, a naphthyl group, a fluorenyl group, a carbazolyl group, a dibenzofuranyl group, or a dibenzothiophenyl group;
a π electron-depleted nitrogen-free cyclic group (e.g., a phenyl group, a naphthyl group, a fluorenyl group, a carbazolyl group, a dibenzofuranyl group, a dibenzothiophenyl group, a biphenyl group, a terphenyl group, and a triphenylenyl group);
a π electron-depleted nitrogen-free cyclic group (e.g., a phenyl group, a naphthyl group, a fluorenyl group, a carbazolyl group, a dibenzofuranyl group, a dibenzothiophenyl group, a biphenyl group, a terphenyl group, and a triphenylenyl group) substituted with at least one deuterium, a hydroxyl group, an amino group, an amidino group, a hydrazine group, a hydrazone group, a carboxylic acid group or a salt thereof, a sulfonic acid group or a salt thereof, a phosphoric acid group or a salt thereof, a C₁-C₂₀ alkyl group, a C₁-C₂₀ alkoxy group, a phenyl group, a naphthyl group, a fluorenyl group, a carbazolyl group, a dibenzofuranyl group, a dibenzothiophenyl group, a biphenyl group, or a combination thereof,

—Si(Q₄₀₄)(Q₄₀₅)(Q₄₀₆),
e1 and e2 may each independently be an integer from 0 to 10,
Q₄₀₁ to Q₄₀₆ may each independently be hydrogen, deuterium, a hydroxyl group, an amino group, an amidino group, a hydrazine group, a hydrazone group, a carboxylic acid group or a salt thereof, a sulfonic acid group or a salt thereof, a phosphoric acid group or a salt thereof, a phenyl group, a naphthyl group, a fluorenyl group, a carbazolyl group, a dibenzofuranyl group, a dibenzothiophenyl group, a biphenyl group, a terphenyl group, or a triphenylenyl group, and
* indicates a binding site to a neighboring atom.

In an embodiment, in Formula E-1, Ar₃₀₁ and L₃₀₁ may each independently be a benzene group, a naphthalene group, a fluorene group, a spiro-bifluorene group, a benzofluorene group, a dibenzofluorene group, a phenalene group, a phenanthrene group, an anthracene group, a fluoranthene group, a triphenylene group, a pyrene group, a chrysene group, a naphthacene group, a picene group, a perylene group, a pentaphene group, an indenoanthracene group, a dibenzofuran group, a dibenzothiophene group, an imidazole group, a pyrazole group, a thiazole group, an isothiazole group, an oxazole group, an isoxazole group, a pyridine group, a pyrazine group, a pyridazine group, a pyrimidine group, an indazole group, a purine group, a quinoline group, an isoquinoline group, a benzoquinoline group, a phthalazine group, a naphthyridine group, a quinoxaline group, a quinazoline group, a cinnoline group, a phenanthridine group, an acridine group, a phenanthroline group, a phenazine group, a benzimidazole group, an isobenzothiazole group, a benzoxazole group, an isobenzoxazole group, a triazole group, a tetrazole group, an oxadiazole group, a triazine group, a thiadiazole group, an imidazopyridine group, an imidazopyrimidine group, or an azacarbazole group, each unsubstituted or substituted with at least one deuterium, —F, —Cl, —Br, —I, a hydroxyl group, a cyano group, a nitro group, an amidino group, a hydrazino group, a hydrazono group, a C₁-C₂₀ alkyl group, a C₁-C₂₀ alkoxy group, a phenyl group, a biphenyl group, a terphenyl group, a naphthyl group, a cyano group-containing phenyl group, a cyano group-containing biphenyl group, a cyano group-containing terphenyl group, a cyano group-containing naphthyl group, a pyridinyl group, a phenylpyridinyl group, a diphenylpyridinyl group, a biphenylpyridinyl group, a di(biphenyl)pyridinyl group, a pyrazinyl group, a phenylpyrazinyl group, a diphenylpyrazinyl group, a biphenylpyrazinyl group, a di(biphenyl)pyrazinyl group, a pyridazinyl group, a phenylpyridazinyl group, a diphenylpyridazinyl group, a biphenylpyridazinyl group, a di(biphenyl)pyridazinyl group, a pyrimidinyl group, a phenylpyrimidinyl group, a diphenylpyrimidinyl group, a biphenylpyrimidinyl group, a di(biphenyl)pyrimidinyl group, a triazinyl group, a phenyltriazinyl group, a diphenyltriazinyl group, a biphenyltriazinyl group, a di(biphenyl)triazinyl group, —Si(Q₃₁)(Q₃₂)(Q₃₃), —N(Q₃₁)(Q₃₂), —B(Q₃₁)(Q₃₂), —C(═O)(Q₃₁), —S(═O)₂(Q₃₁), —P(═O)(Q₃₁)(Q₃₂), or a combination thereof, at least one of L₃₀₁(s) in the number of xb1 may each independently be an imidazole group, a pyrazole group, a thiazole group, an isothiazole group, an oxazole group, an isoxazole group, a pyridine group, a pyrazine group, a pyridazine group, a pyrimidine group, an indazole group, a purine group, a quinoline group, an isoquinoline group, a benzoquinoline group, a phthalazine group, a naphthyridine group, a quinoxaline group, a quinazoline group, a cinnoline group, a phenanthridine group, an acridine group, a phenanthroline group, a phenazine group, a benzimidazole group, an isobenzothiazole group, a benzoxazole group, an isobenzoxazole group, a triazole group, a tetrazole group, an oxadiazole group, a triazine group, a thiadiazole group, an imidazopyridine group, an imidazopyrimidine group, or an azacarbazole group, each unsubstituted or substituted with at least one deuterium, —F, —Cl, —Br, —I, a hydroxyl group, a cyano group, a nitro group, an amidino group, a hydrazine group, a hydrazone group, a C₁-C₂₀ alkyl group, a C₁-C₂₀ alkoxy group, a phenyl group, a biphenyl group, a terphenyl group, a naphthyl group, a cyano-containing phenyl group, a cyano-containing biphenyl group, a cyano-containing terphenyl group, a cyano-containing naphthyl group, a pyridinyl group, a phenylpyridinyl group, a diphenylpyridinyl group, a biphenylpyridinyl group, a di(biphenyl)pyridinyl group, a pyrazinyl group, a phenylpyrazinyl group, a diphenylpyrazinyl group, a biphenylpyrazinyl group, a di(biphenyl)pyrazinyl group, a pyridazinyl group, a phenylpyridazinyl group, a diphenylpyridazinyl group, a biphenylpyridazinyl group, a di(biphenyl)pyridazinyl group, a pyrimidinyl group, a phenylpyrimidinyl group, a diphenylpyrimidinyl group, a biphenylpyrimidinyl group, a di(biphenyl)pyrimidinyl group, a triazinyl group, a phenyltriazinyl group, a diphenyltriazinyl group, a biphenyltriazinyl group, a di(biphenyl)triazinyl group, —Si(Q₃₁)(Q₃₂)(Q₃₃), —N(Q₃₁)(Q₃₂), —B(Q₃₁)(Q₃₂), —C(═O)(Q₃₁), —S(═O)₂(Q₃₁), —P(═O)(Q₃₁)(Q₃₂), or a combination thereof, and
R₃₀₁ may be hydrogen, deuterium, —F, —Cl, —Br, —I, a hydroxyl group, a cyano group, a nitro group, an amidino group, a hydrazino group, a hydrazono group, a C₁-C₂₀ alkyl group, a C₁-C₂₀ alkoxy group, a phenyl group, a biphenyl group, a terphenyl group, a tetraphenyl group, a naphthyl group, a cyano group-containing phenyl group, a cyano group-containing biphenyl group, a cyano group-containing terphenyl group, a cyano group-containing tetraphenyl group, a cyano group-containing naphthyl group, a pyridinyl group, a phenylpyridinyl group, a diphenylpyridinyl group, a biphenylpyridinyl group, a di(biphenyl)pyridinyl group, a pyrazinyl group, a phenylpyrazinyl group, a diphenylpyrazinyl group, a biphenylpyrazinyl group, a di(biphenyl)pyrazinyl group, a pyridazinyl group, a phenylpyridazinyl group, a diphenylpyridazinyl group, a biphenylpyridazinyl group, a di(biphenyl)pyridazinyl group, a pyrimidinyl group, a phenylpyrimidinyl group, a diphenylpyrimidinyl group, a biphenylpyrimidinyl group, a di(biphenyl)pyrimidinyl group, a triazinyl group, a phenyltriazinyl group, a diphenyltriazinyl group, a biphenyltriazinyl group, a di(biphenyl)triazinyl group, —Si(Q₃₁)(Q₃₂)(Q₃₃), —N(Q₃₁)(Q₃₂), —B(Q₃₁)(Q₃₂), —C(═O)(Q₃₁), —S(═O)₂(Q₃₁), or —P(═O)(Q₃₁)(Q₃₂), and
Q₃₁ to Q₃₃ may each independently be a C₁-C₁₀ alkyl group, a C₁-C₁₀ alkoxy group, a phenyl group, a biphenyl group, a terphenyl group, or a naphthyl group, but embodiments of the present disclosure are not limited thereto.

In one or more embodiments,
Ar₃₀₁ may be a benzene group, a naphthalene group, a fluorene group, a spiro-bifluorene group, a benzofluorene group, a dibenzofluorene group, a phenalene group, a phenanthrene group, an anthracene group, a fluoranthene group, a triphenylene group, a pyrene group, a chrysene group, a naphthacene group, a picene group, a perylene group, a pentaphene group, an indenoanthracene group, a dibenzofuran group, or a dibenzothiophene group, each unsubstituted or substituted with at least one deuterium, —F, —Cl, —Br, —I, a hydroxyl group, a cyano group, a nitro group, an amidino group, a hydrazine group, a hydrazone group, a C₁-C₂₀ alkyl group, a C₁-C₂₀ alkoxy group, a phenyl group, a biphenyl group, a terphenyl group, a naphthyl group, a cyano-containing phenyl group, a cyano-containing biphenyl group, a cyano-containing terphenyl group, a cyano-containing naphthyl group, a pyridinyl group, a phenylpyridinyl group, a diphenylpyridinyl group, a biphenylpyridinyl group, a di(biphenyl)pyridinyl group, a pyrazinyl group, a phenylpyrazinyl group, a diphenylpyrazinyl group, a biphenylpyrazinyl group, a di(biphenyl)pyrazinyl group, a pyridazinyl group, a phenylpyridazinyl group, a diphenylpyridazinyl group, a biphenylpyridazinyl group, a di(biphenyl)pyridazinyl group, a pyrimidinyl group, a phenylpyrimidinyl group, a diphenylpyrimidinyl group, a biphenylpyrimidinyl group, a di(biphenyl)pyrimidinyl group, a triazinyl group, a phenyltriazinyl group, a diphenyltriazinyl group, a biphenyltriazinyl group, a di(biphenyl)triazinyl group, —Si(Q₃₁)(Q₃₂)(Q₃₃), —N(Q₃₁)(Q₃₂), —B(Q₃₁)(Q₃₂), —C(═O)(Q₃₁), —S(═O)₂(Q₃₁), —P(═O)(Q₃₁)(Q₃₂), or a combination thereof; or
a group represented by Formulae 5-1 to 5-3 and 6-1 to 6-33, and
L₃₀₁ may be a group represented by one of Formulae 5-1 to 5-3 and 6-1 to 6-33:

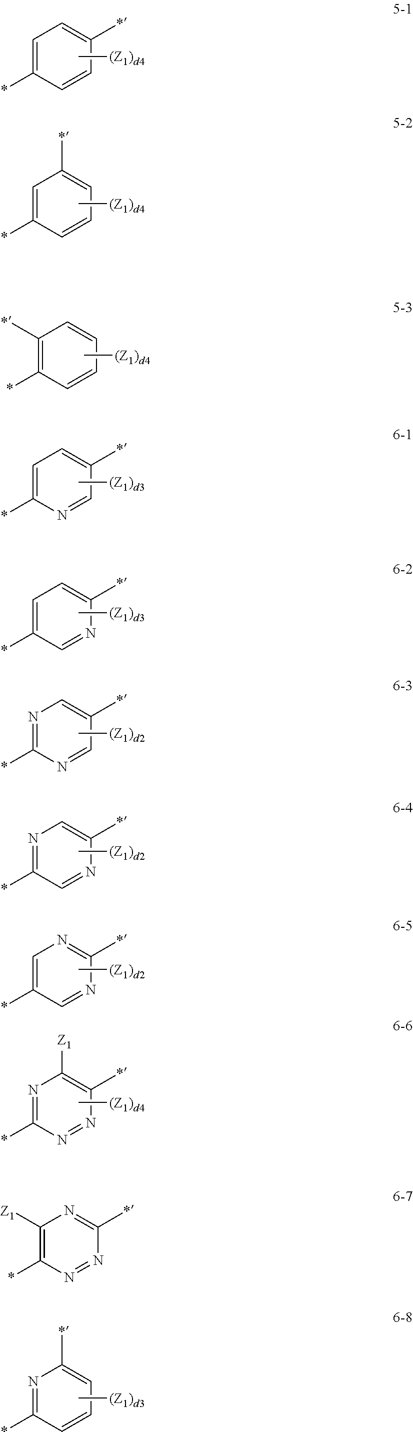

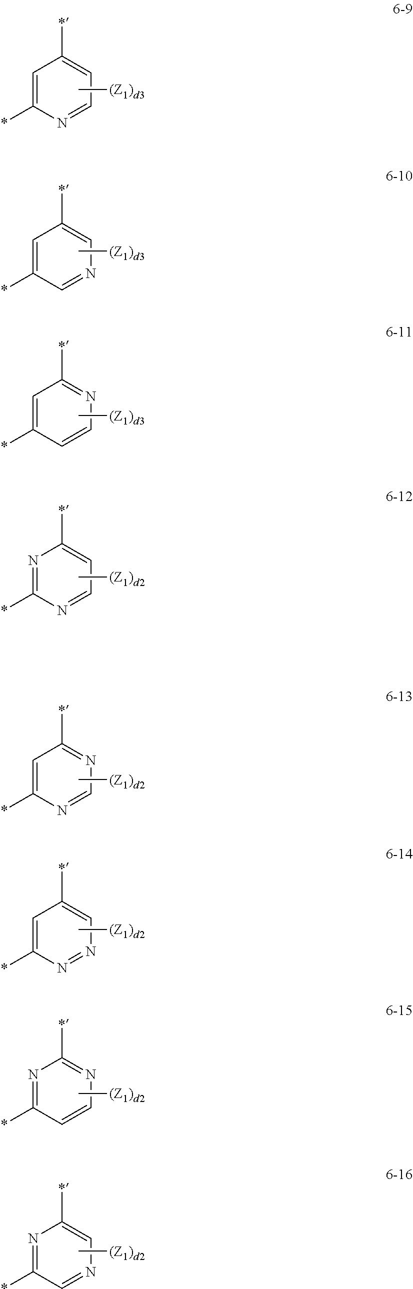

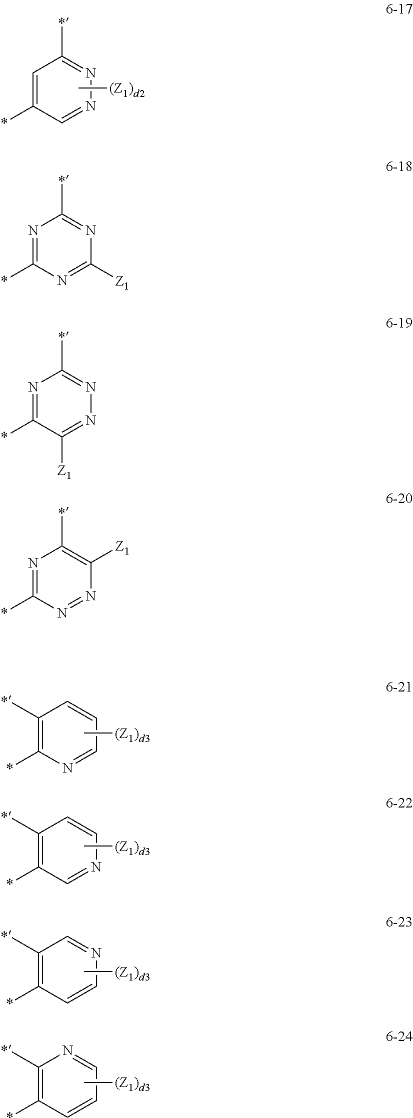

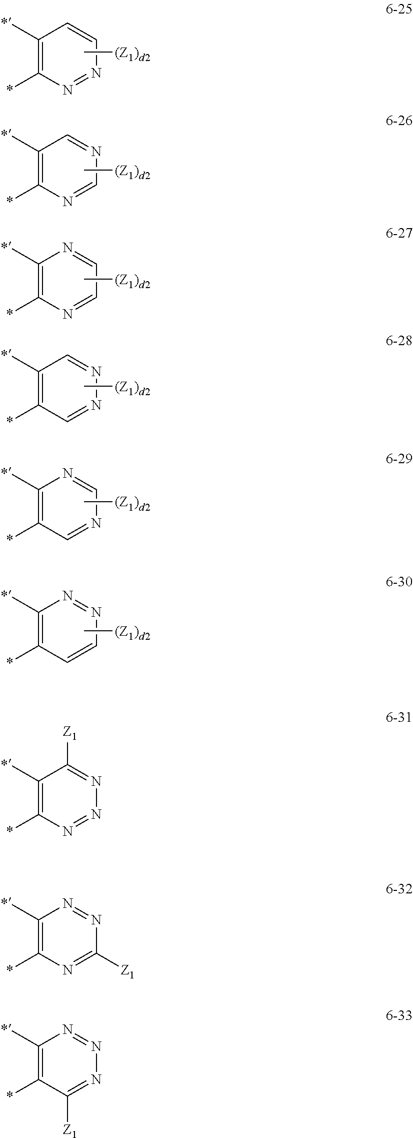

wherein, in Formulae 5-1 to 5-3 and 6-1 to 6-33,
Z₁ may be hydrogen, deuterium, —F, —Cl, —Br, —I, a hydroxyl group, a cyano group, a nitro group, an amidino group, a hydrazino group, a hydrazono group, a C₁-C₂₀ alkyl group, a C₁-C₂₀ alkoxy group, a phenyl group, a biphenyl group, a terphenyl group, a naphthyl group, a cyano group-containing phenyl group, a cyano group-containing biphenyl group, a cyano group-containing terphenyl group, a cyano group-containing naphthyl group, a pyridinyl group, a phenylpyridinyl group, a diphenylpyridinyl group, a biphenylpyridinyl group, a di(biphenyl)pyridinyl group, a pyrazinyl group, a phenylpyrazinyl group, a diphenylpyrazinyl group, a biphenylpyrazinyl group, a di(biphenyl)pyrazinyl group, a pyridazinyl group, a phenylpyridazinyl group, a diphenylpyridazinyl group, a biphenylpyridazinyl group, a di(biphenyl)pyridazinyl group, a pyrimidinyl group, a phenylpyrimidinyl group, a diphenylpyrimidinyl group, a biphenylpyrimidinyl group, a di(biphenyl)pyrimidinyl group, a triazinyl group, a phenyltriazinyl group, a diphenyltriazinyl group, a biphenyltriazinyl group, a di(biphenyl)triazinyl group, —Si(Q₃₁)(Q₃₂)(Q₃₃), —N(Q₃₁)(Q₃₂), —B(Q₃₁)(Q₃₂), —C(═O)(Q₃₁), —S(═O)₂(Q₃₁), or —P(═O)(Q₃₁)(Q₃₂),
d4 may be 0, 1, 2, 3, or 4,
d3 may be 0, 1, 2, or 3,
d2 may be 0, 1, or 2,
* and *1 each indicate a binding site to an adjacent atom.

Q₃₁ to Q₃₃ may each be the same as described in the present specification.

In one or more embodiments, L₃₀₁ may be a group represented by one of Formulae 5-2, 5-3 and 6-8 to 6-33.

In one or more embodiments, R₃₀₁ may be a cyano group or a group represented by Formula 7-1 to 7-18, and at least one of Ar₄₀₂(s) in the number of xd11 may be a group represented by Formulae 7-1 to 7-18, but embodiments of the present disclosure are not limited thereto:

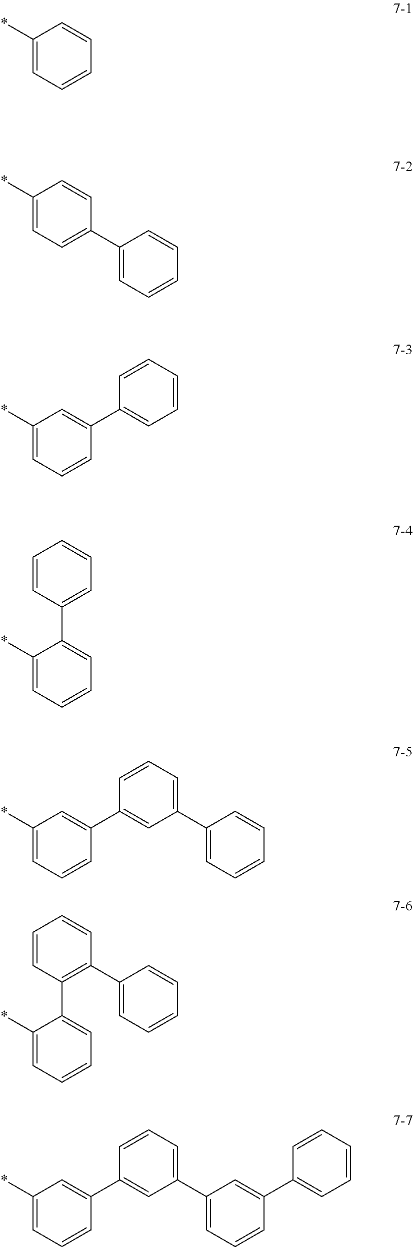

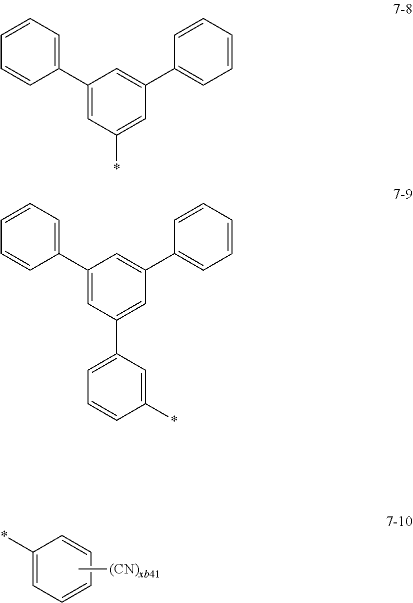

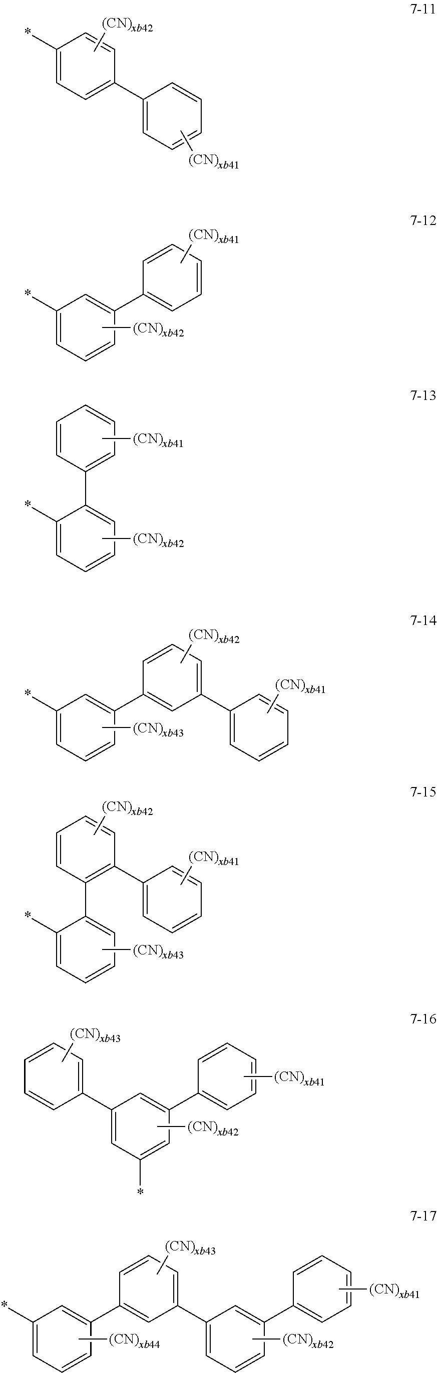

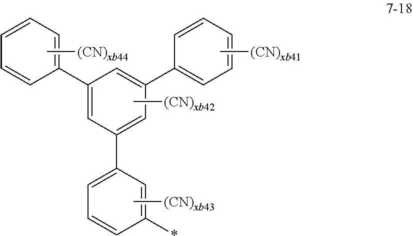

wherein, in Formulae 7-1 to 7-18,
xb41 to xb44 may each be 0, 1, or 2, provided that xb41 in Formula 7-10 may not be 0, xb41+xb42 in Formulae 7-11 to 7-13 may not be 0, xb41+xb42+xb43 in Formulae 7-14 to 7-16 may not be 0, xb41+xb42+xb43+xb44 in Formulae 7-17 and 7-18 may not be 0, and * indicates a binding site to an adjacent atom.

In Formula E-1, at least two Ar₃₀₁(s) may be identical to or different from each other, and at least two L₃₀₁ (s) may be identical to or different from each other. In Formula H-1, at least two L₄₀₁(s) may be identical to or different from each other, and at least two Ar₄₀₂(s) may be identical to or different from each other.

In an embodiment, the electron-transporting host includes i) at least one of a cyano group, a pyrimidine group, a pyrazine group, and a triazine group and ii) a triphenylene group, and the hole-transporting host may include a carbazole group.

In one or more embodiments, the electron-transporting host may include at least one cyano group.

The electron transporting host may be selected from, for example, compounds of Groups HE1 to HE7, but embodiments are not limited thereto:

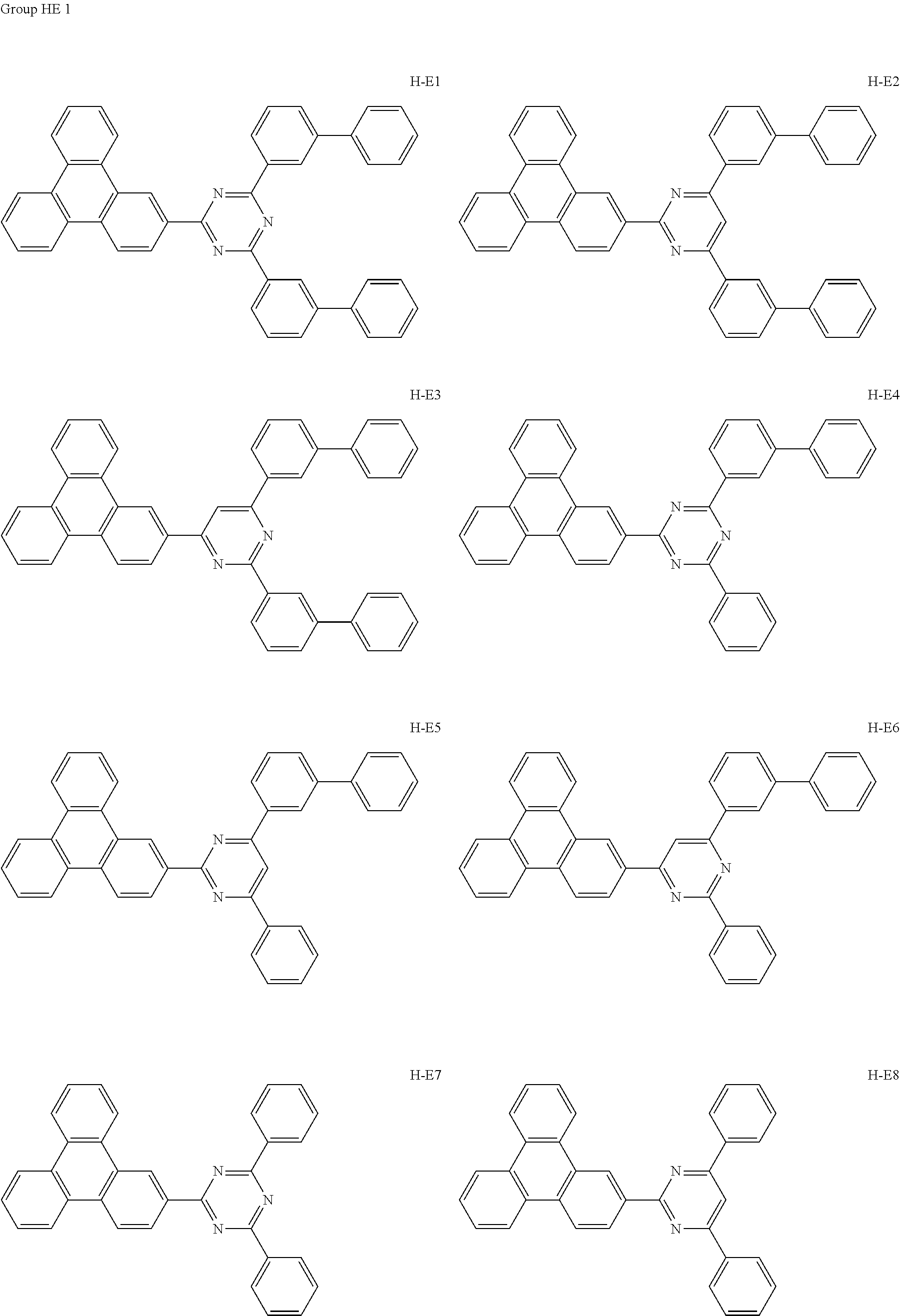

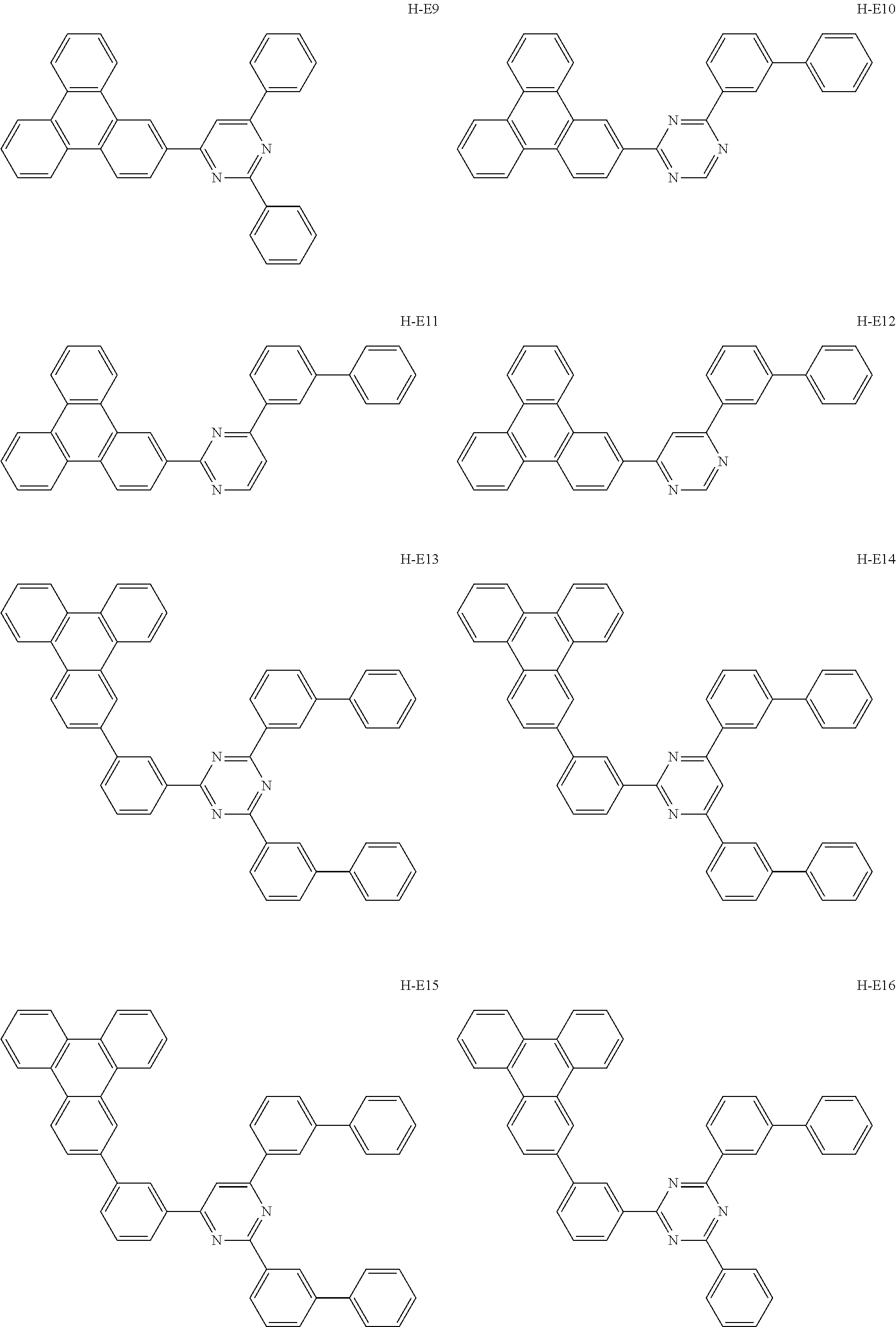

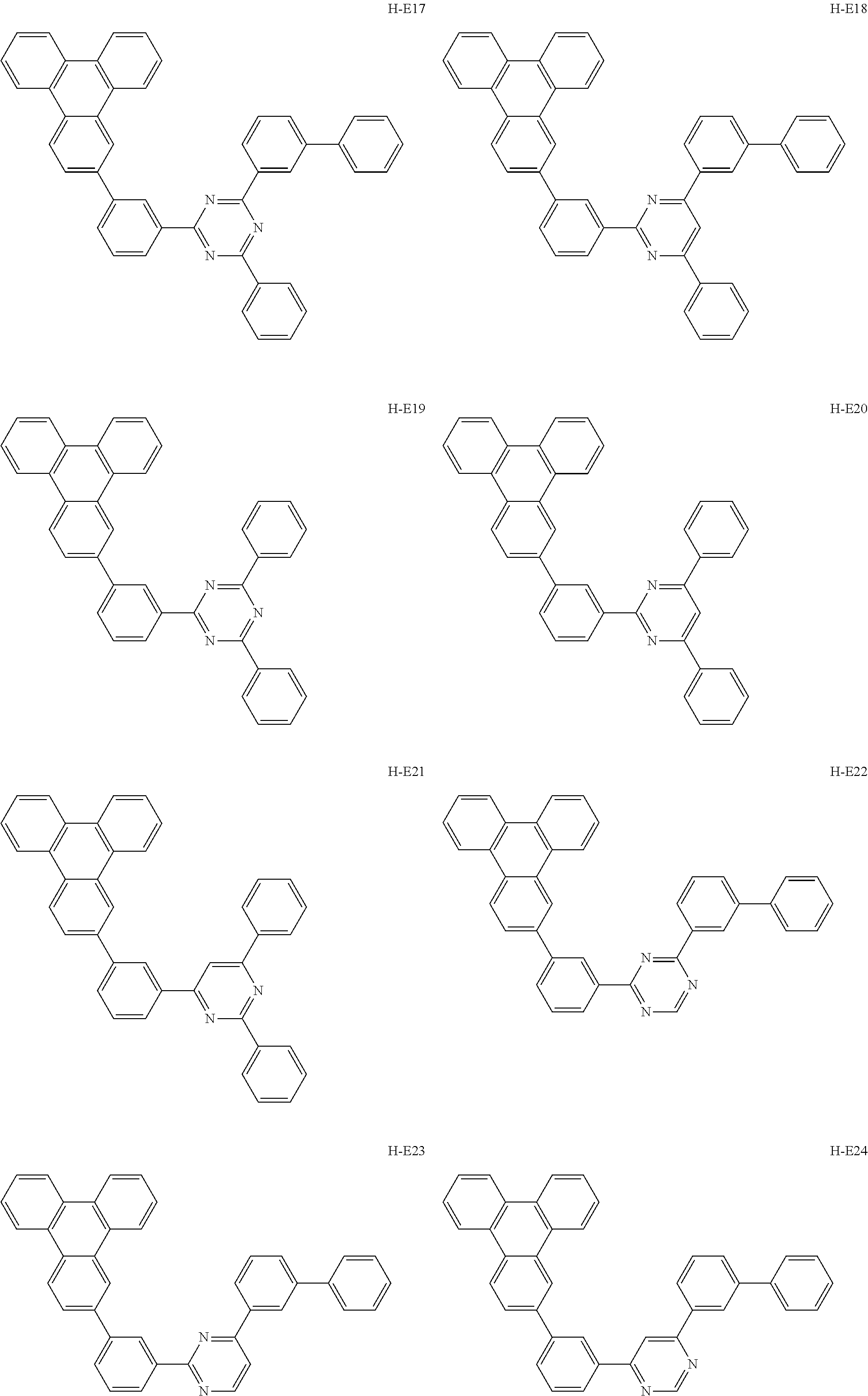

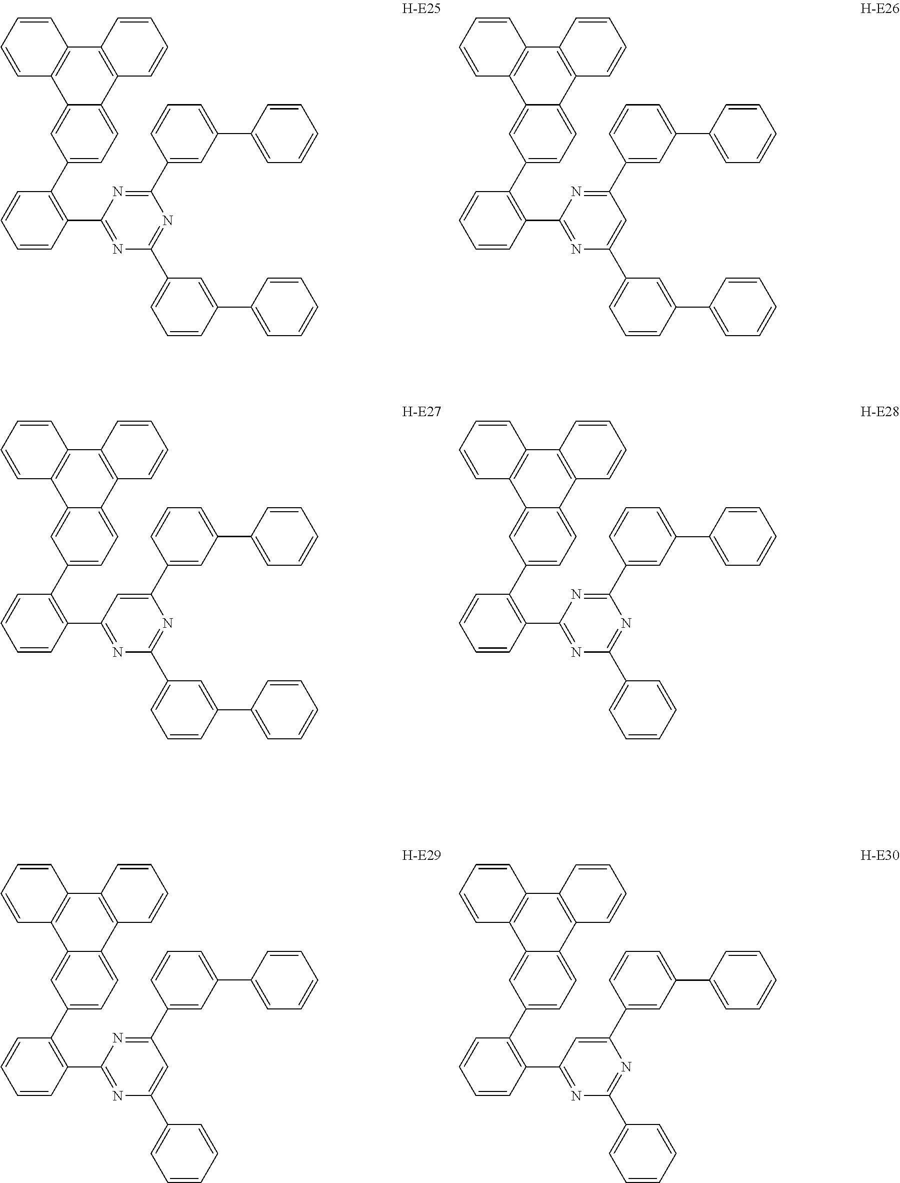

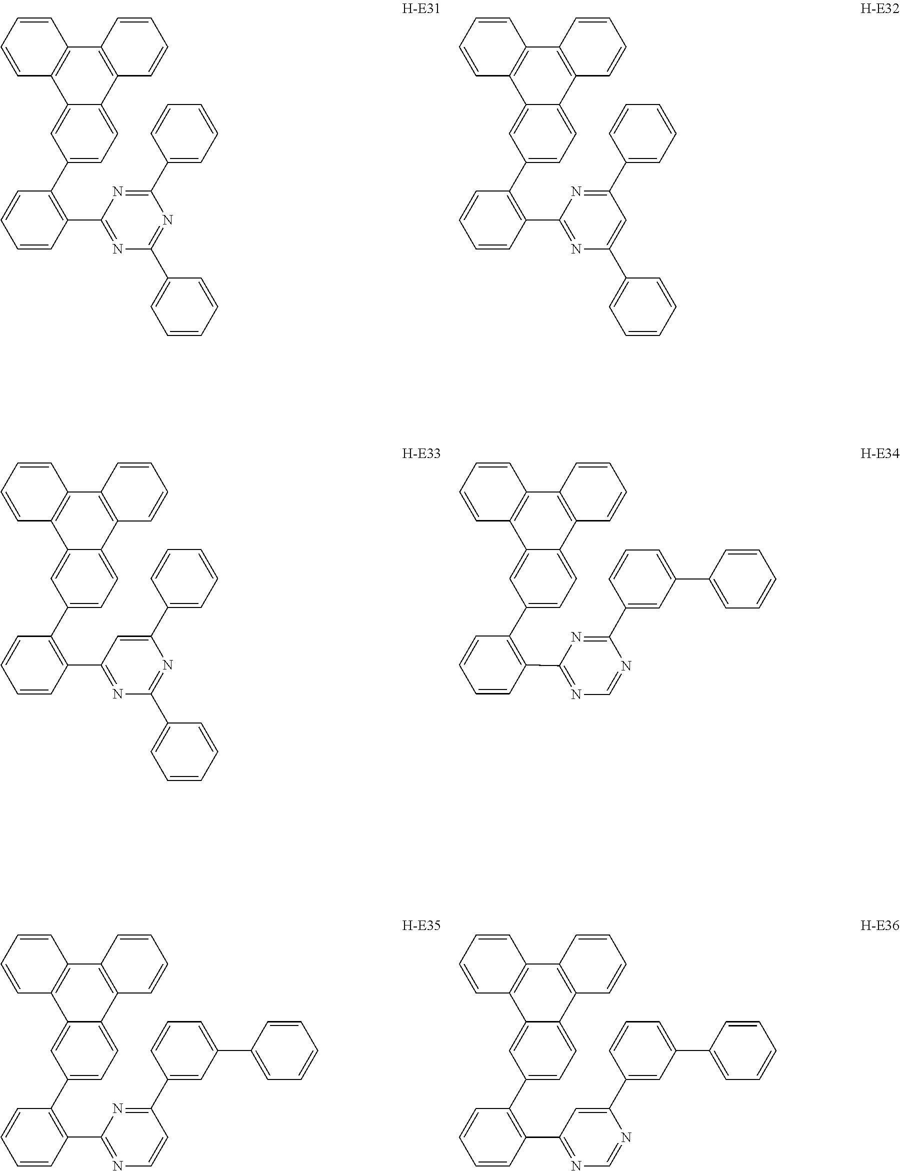 [SMILES: c1ccc(-c2nc(-c3ccccc3)nc(-c3ccccc3-c3ccc4c5ccccc5c5ccccc5c4c3)n2)cc1 ; c1ccc(-c2cc(-c3ccccc3)nc(-c3ccccc3-c3ccc4c5ccccc5c5ccccc5c4c3)n2)cc1 ; c1ccc(-c2cc(-c3ccccc3-c3ccc4c5ccccc5c5ccccc5c4c3)nc(-c3ccccc3)n2)cc1 ; c1ccc(-c2cccc(-c3ncnc(-c4ccccc4-c4ccc5c6ccccc6c6ccccc6c5c4)n3)c2)cc1 ; c1ccc(-c2cccc(-c3ccnc(-c4ccccc4-c4ccc5c6ccccc6c6ccccc6c5c4)n3)c2)cc1 ; c1ccc(-c2cccc(-c3cc(-c4ccccc4-c4ccc5c6ccccc6c6ccccc6c5c4)ncn3)c2)cc1]

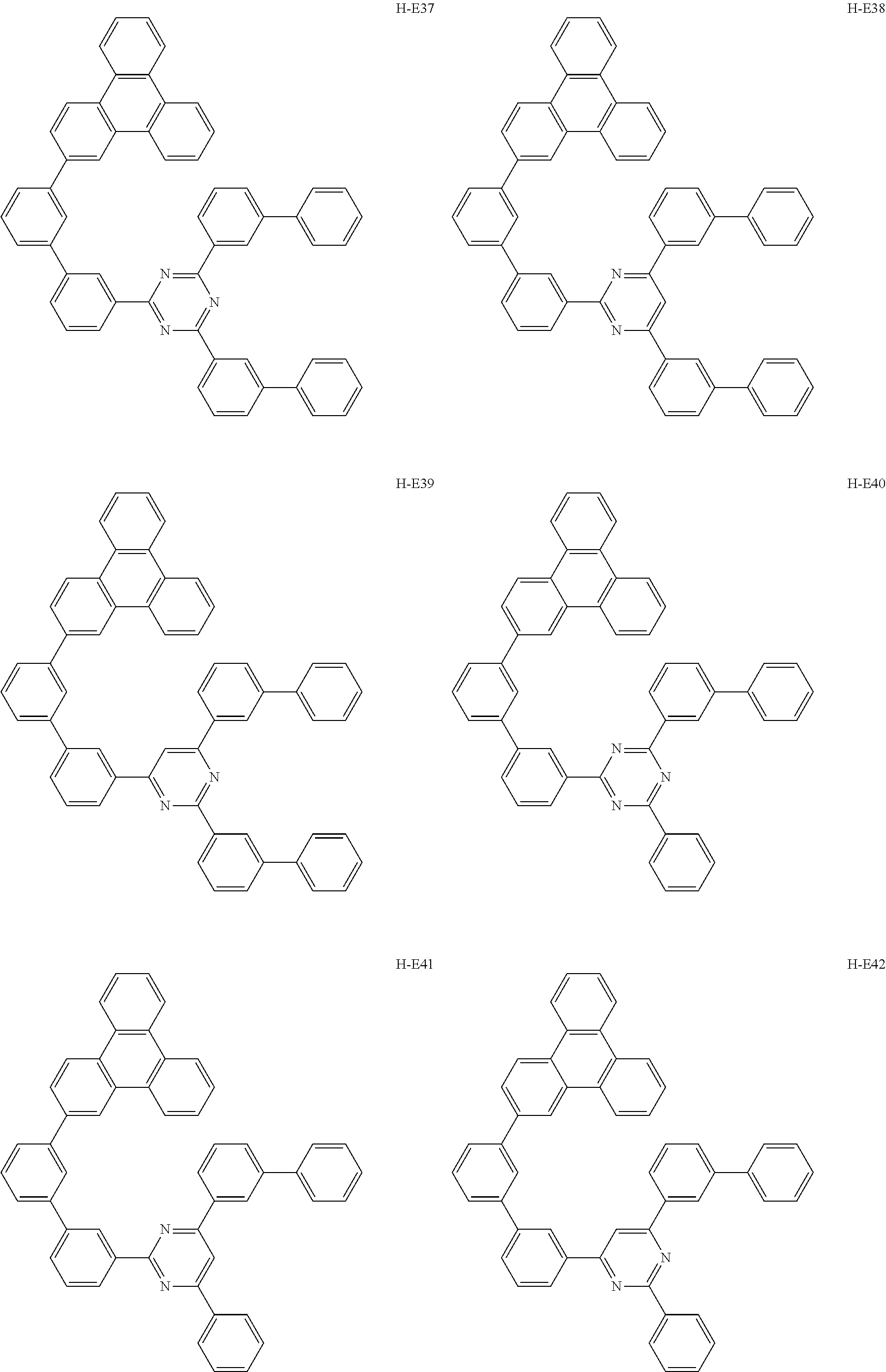

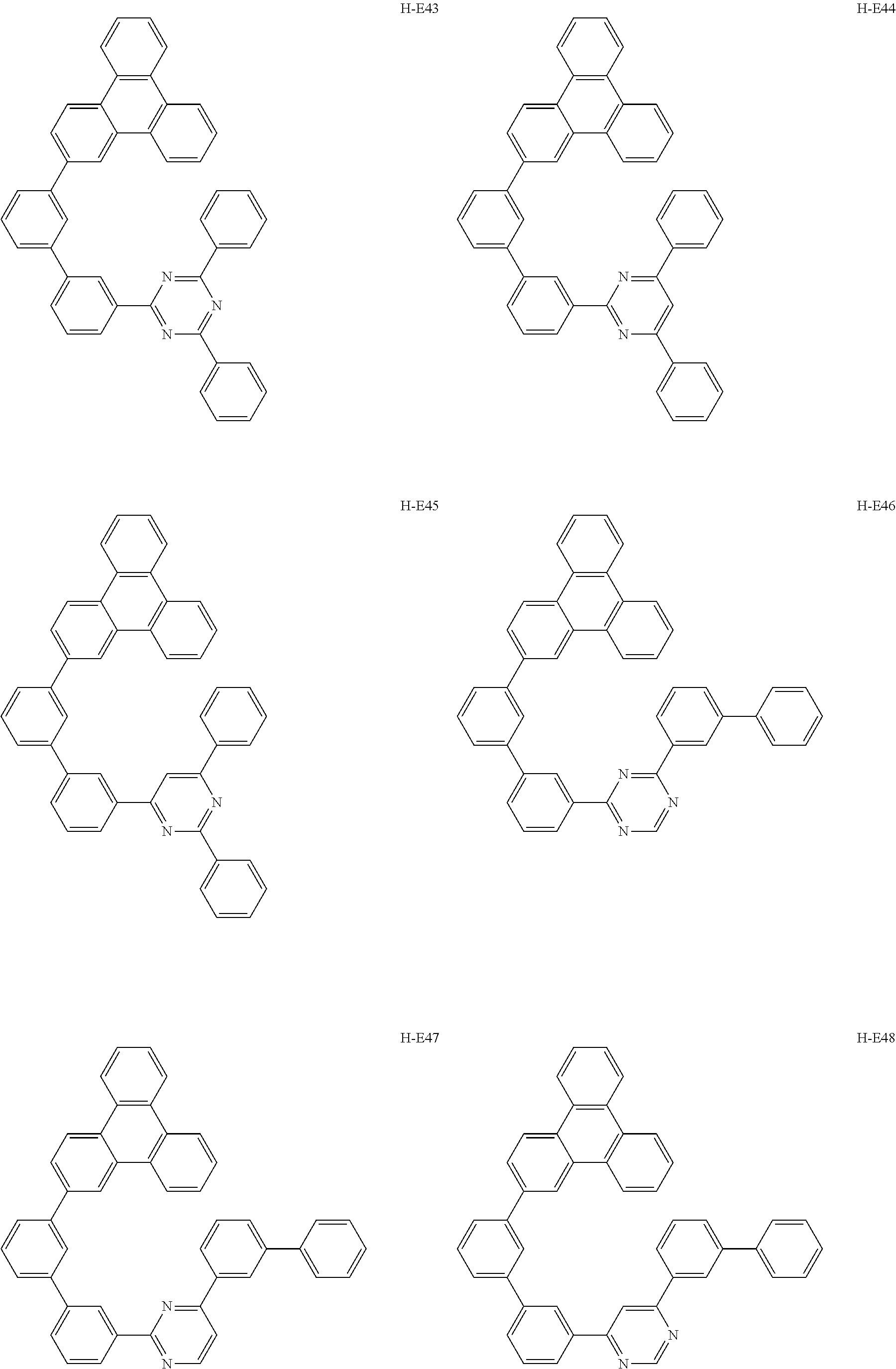

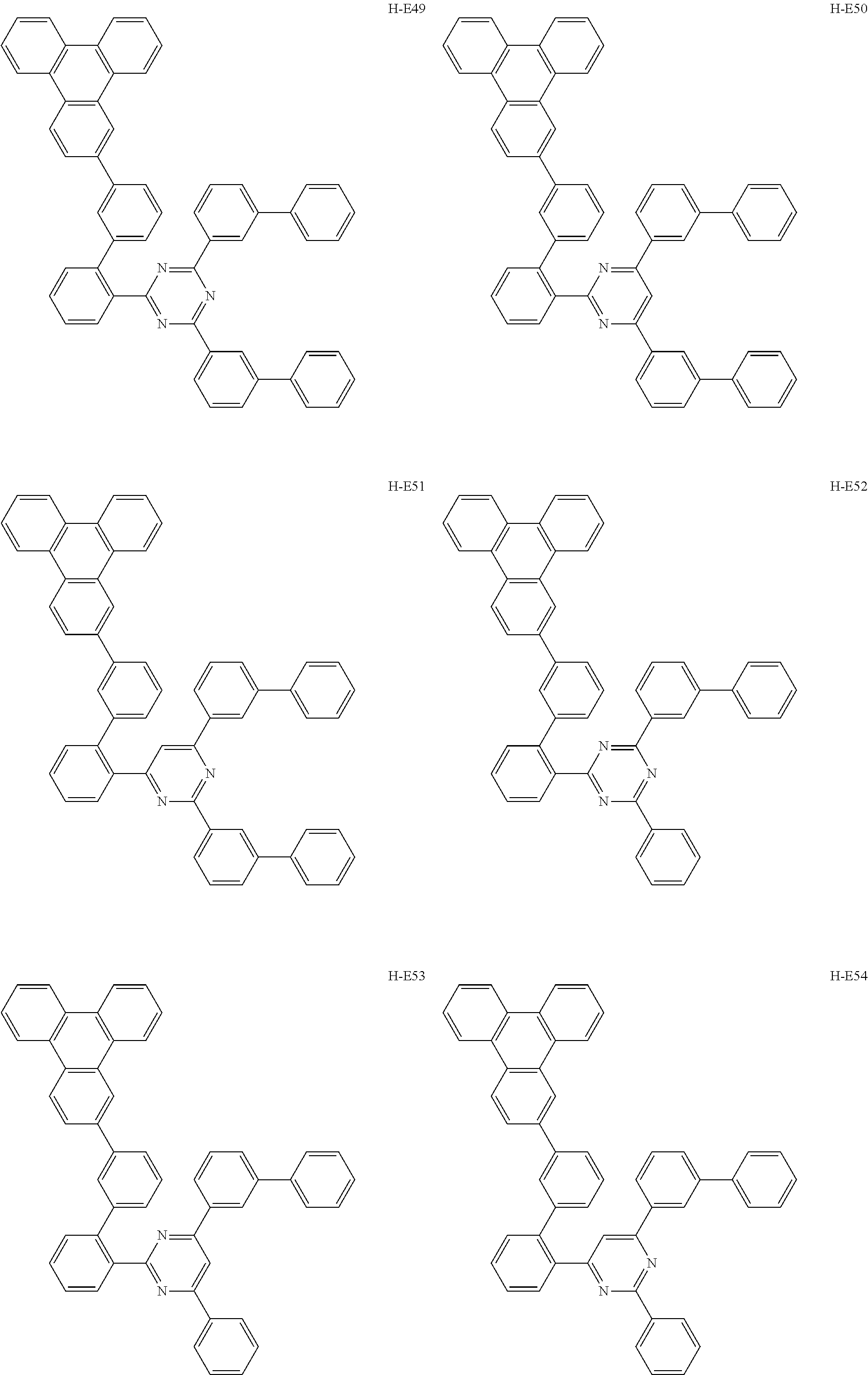

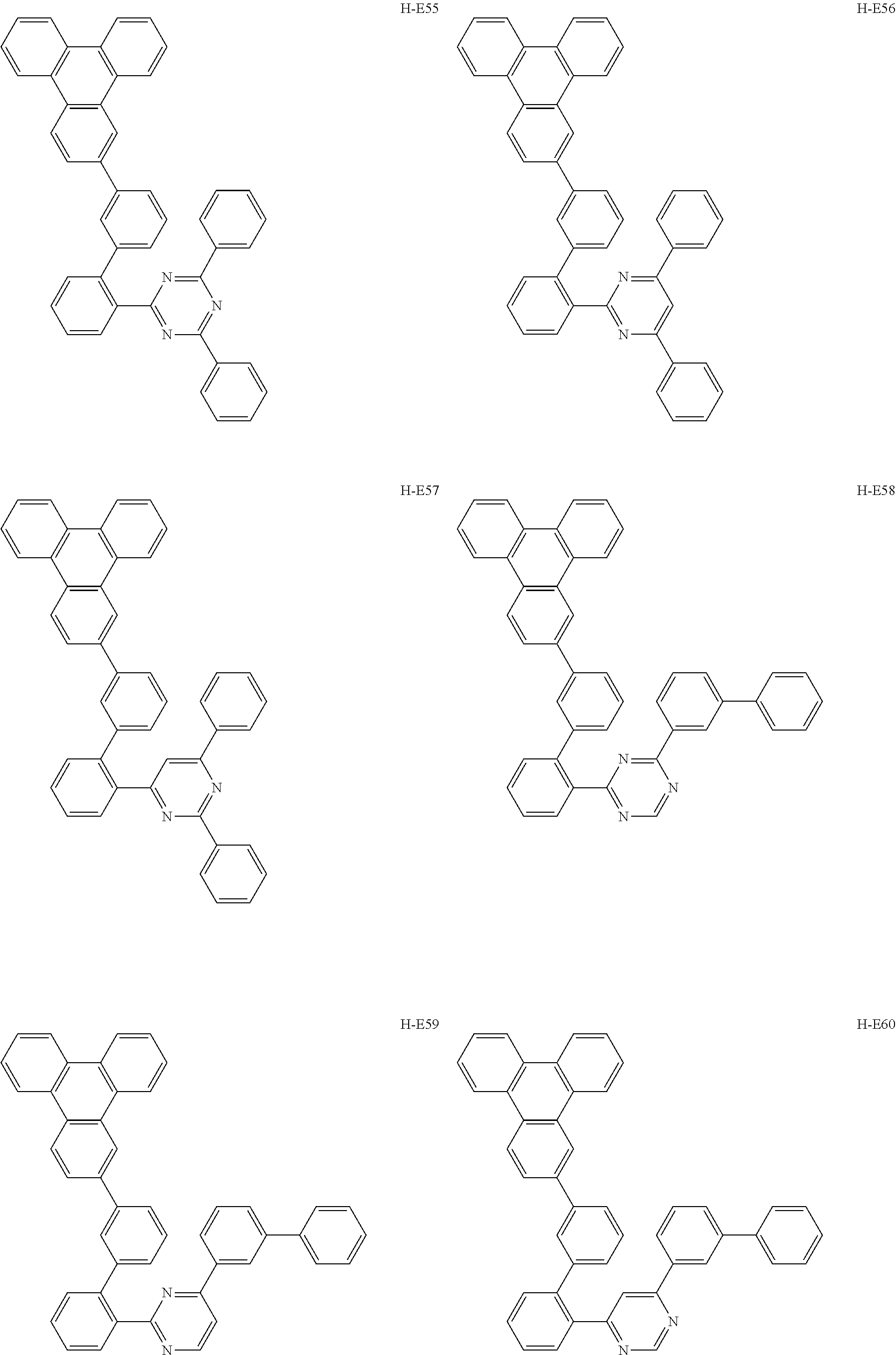 [SMILES: c1ccc(-c2nc(-c3ccccc3)nc(-c3ccccc3-c3cccc(-c4ccc5c6ccccc6c6ccccc6c5c4)c3)n2)cc1 ; c1ccc(-c2cc(-c3ccccc3)nc(-c3ccccc3-c3cccc(-c4ccc5c6ccccc6c6ccccc6c5c4)c3)n2)cc1 ; c1ccc(-c2cc(-c3ccccc3-c3cccc(-c4ccc5c6ccccc6c6ccccc6c5c4)c3)nc(-c3ccccc3)n2)cc1 ; c1ccc(-c2cccc(-c3ncnc(-c4ccccc4-c4cccc(-c5ccc6c7ccccc7c7ccccc7c6c5)c4)n3)c2)cc1 ; c1ccc(-c2cccc(-c3ccnc(-c4ccccc4-c4cccc(-c5ccc6c7ccccc7c7ccccc7c6c5)c4)n3)c2)cc1 ; c1ccc(-c2cccc(-c3cc(-c4ccccc4-c4cccc(-c5ccc6c7ccccc7c7ccccc7c6c5)c4)ncn3)c2)cc1]

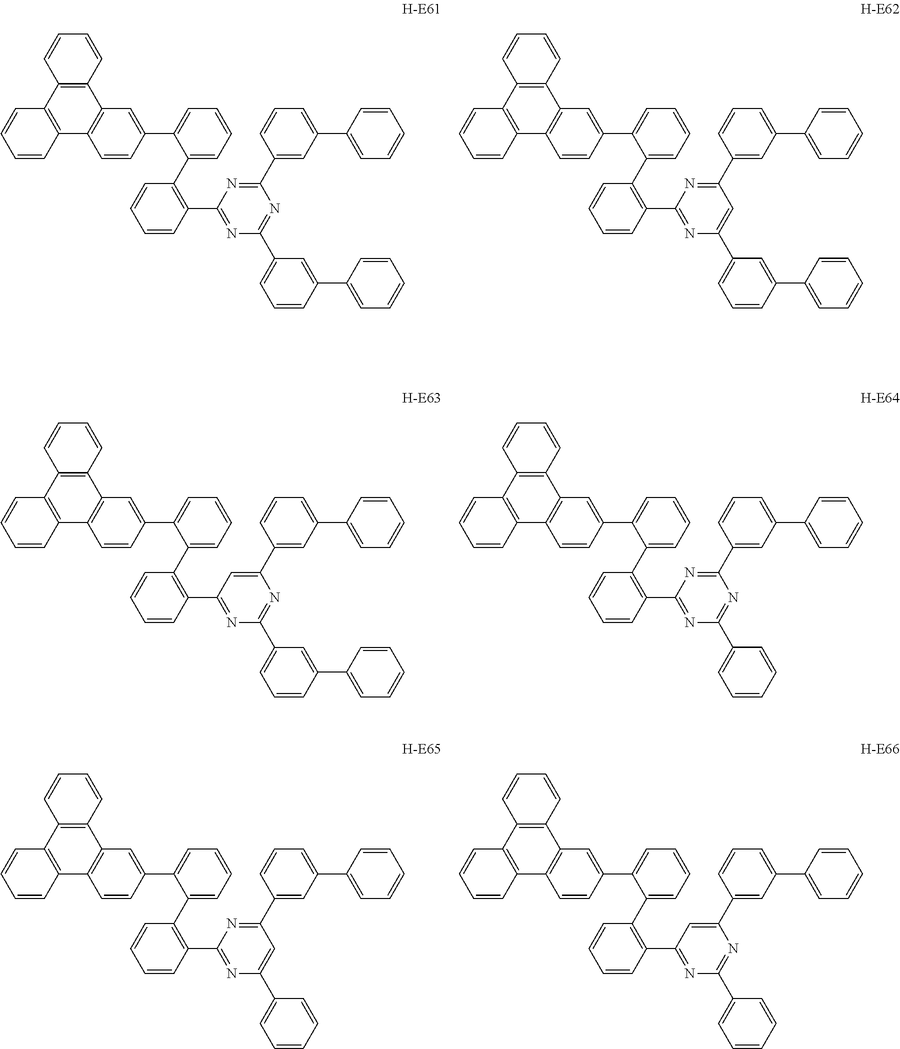

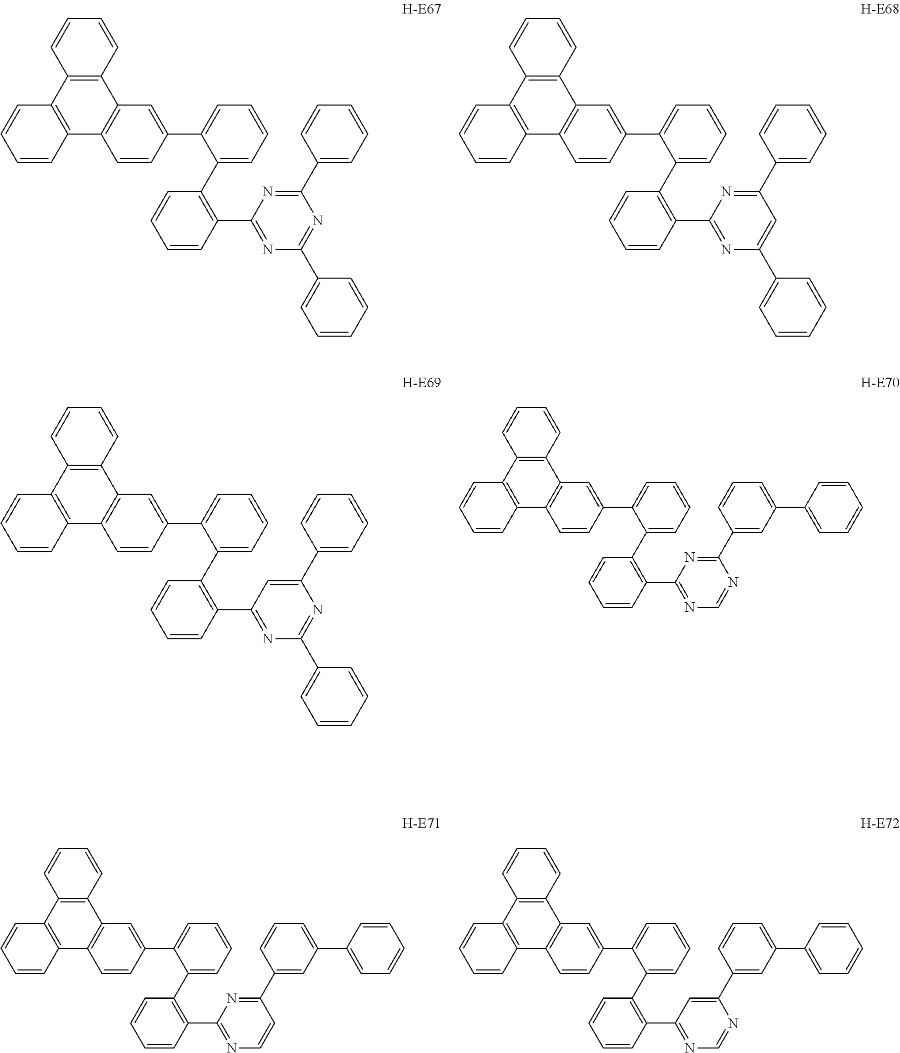 [SMILES: c1ccc(-c2nc(-c3ccccc3)nc(-c3ccccc3-c3ccccc3-c3ccc4c5ccccc5c5ccccc5c4c3)n2)cc1 ; c1ccc(-c2cc(-c3ccccc3)nc(-c3ccccc3-c3ccccc3-c3ccc4c5ccccc5c5ccccc5c4c3)n2)cc1 ; c1ccc(-c2cc(-c3ccccc3-c3ccccc3-c3ccc4c5ccccc5c5ccccc5c4c3)nc(-c3ccccc3)n2)cc1 ; c1ccc(-c2cccc(-c3ncnc(-c4ccccc4-c4ccccc4-c4ccc5c6ccccc6c6ccccc6c5c4)n3)c2)cc1 ; c1ccc(-c2cccc(-c3ccnc(-c4ccccc4-c4ccccc4-c4ccc5c6ccccc6c6ccccc6c5c4)n3)c2)cc1 ; c1ccc(-c2cccc(-c3cc(-c4ccccc4-c4ccccc4-c4ccc5c6ccccc6c6ccccc6c5c4)ncn3)c2)cc1]

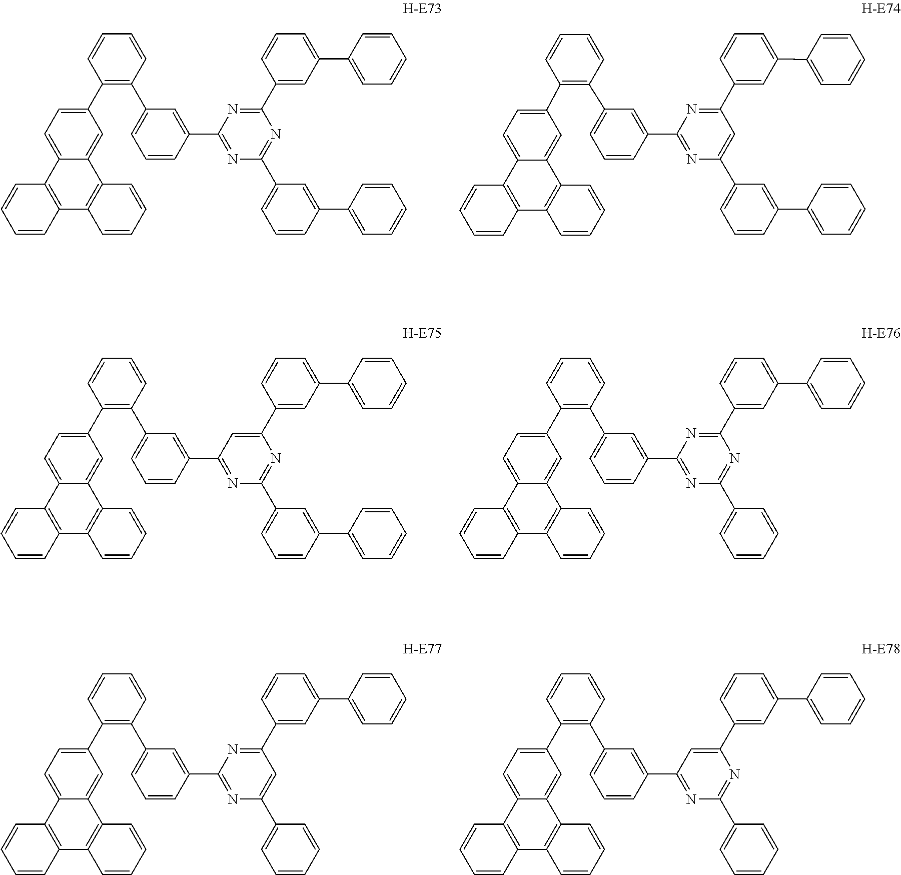

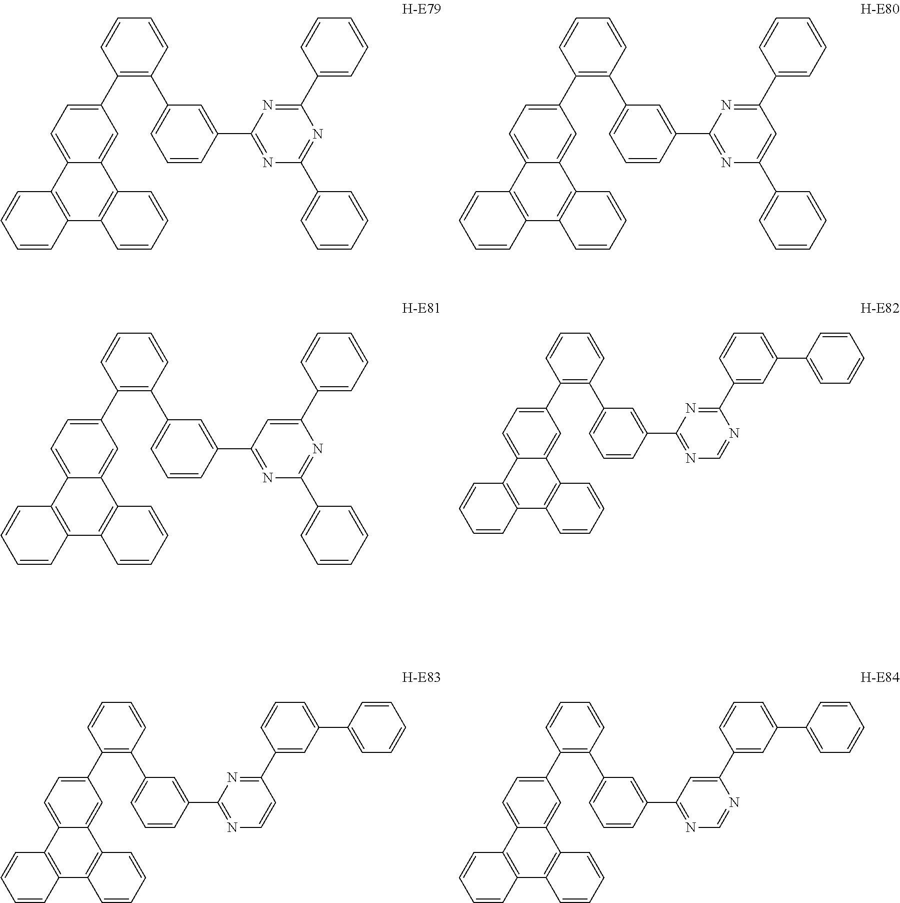 [SMILES: c1ccc(-c2nc(-c3ccccc3)nc(-c3cccc(-c4ccccc4-c4ccc5c6ccccc6c6ccccc6c5c4)c3)n2)cc1 ; c1ccc(-c2cc(-c3ccccc3)nc(-c3cccc(-c4ccccc4-c4ccc5c6ccccc6c6ccccc6c5c4)c3)n2)cc1 ; c1ccc(-c2cc(-c3cccc(-c4ccccc4-c4ccc5c6ccccc6c6ccccc6c5c4)c3)nc(-c3ccccc3)n2)cc1 ; c1ccc(-c2cccc(-c3ncnc(-c4cccc(-c5ccccc5-c5ccc6c7ccccc7c7ccccc7c6c5)c4)n3)c2)cc1 ; c1ccc(-c2cccc(-c3ccnc(-c4cccc(-c5ccccc5-c5ccc6c7ccccc7c7ccccc7c6c5)c4)n3)c2)cc1 ; c1ccc(-c2cccc(-c3cc(-c4cccc(-c5ccccc5-c5ccc6c7ccccc7c7ccccc7c6c5)c4)ncn3)c2)cc1]

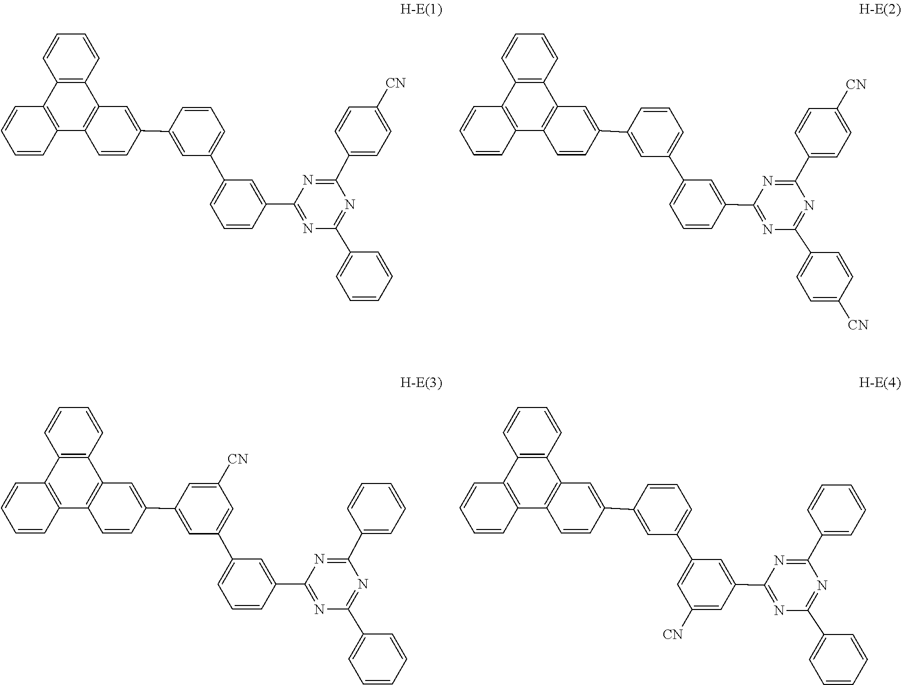

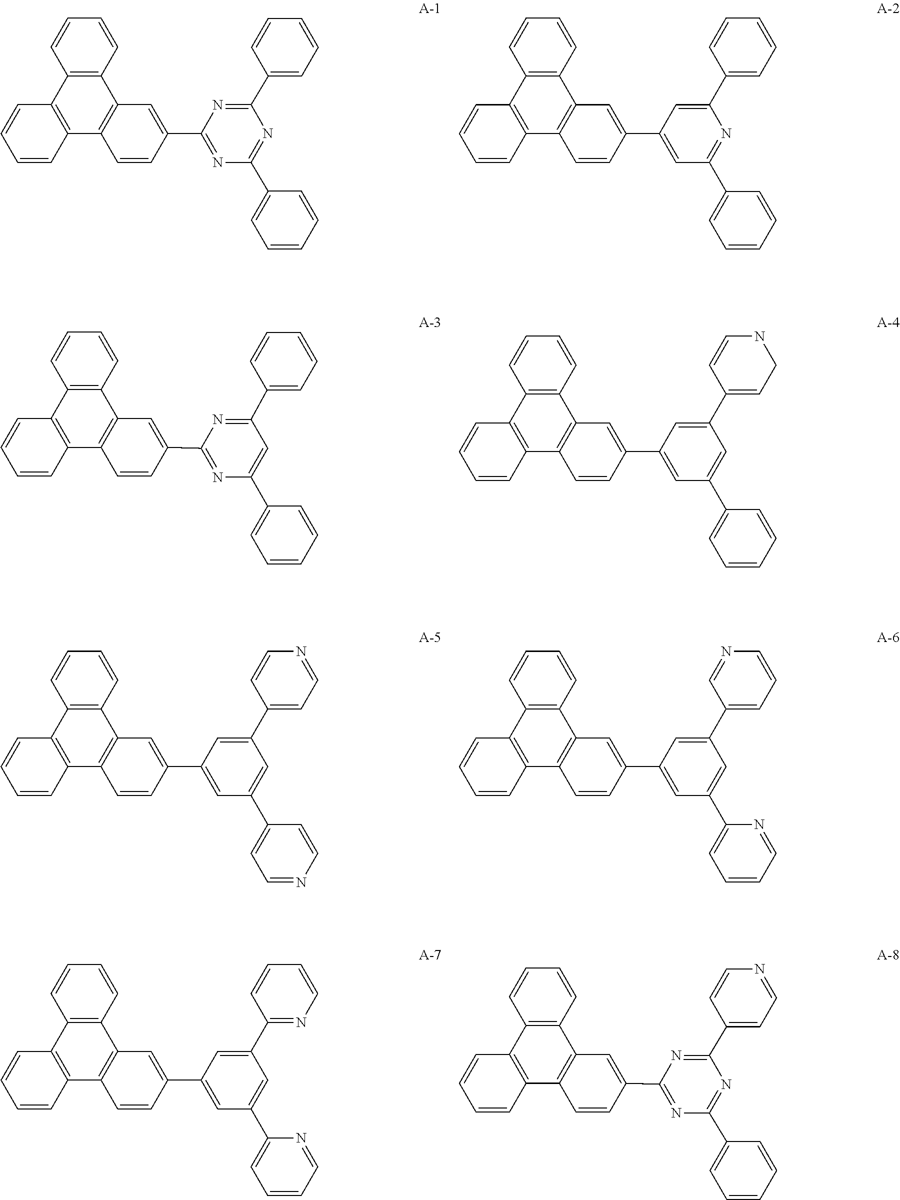

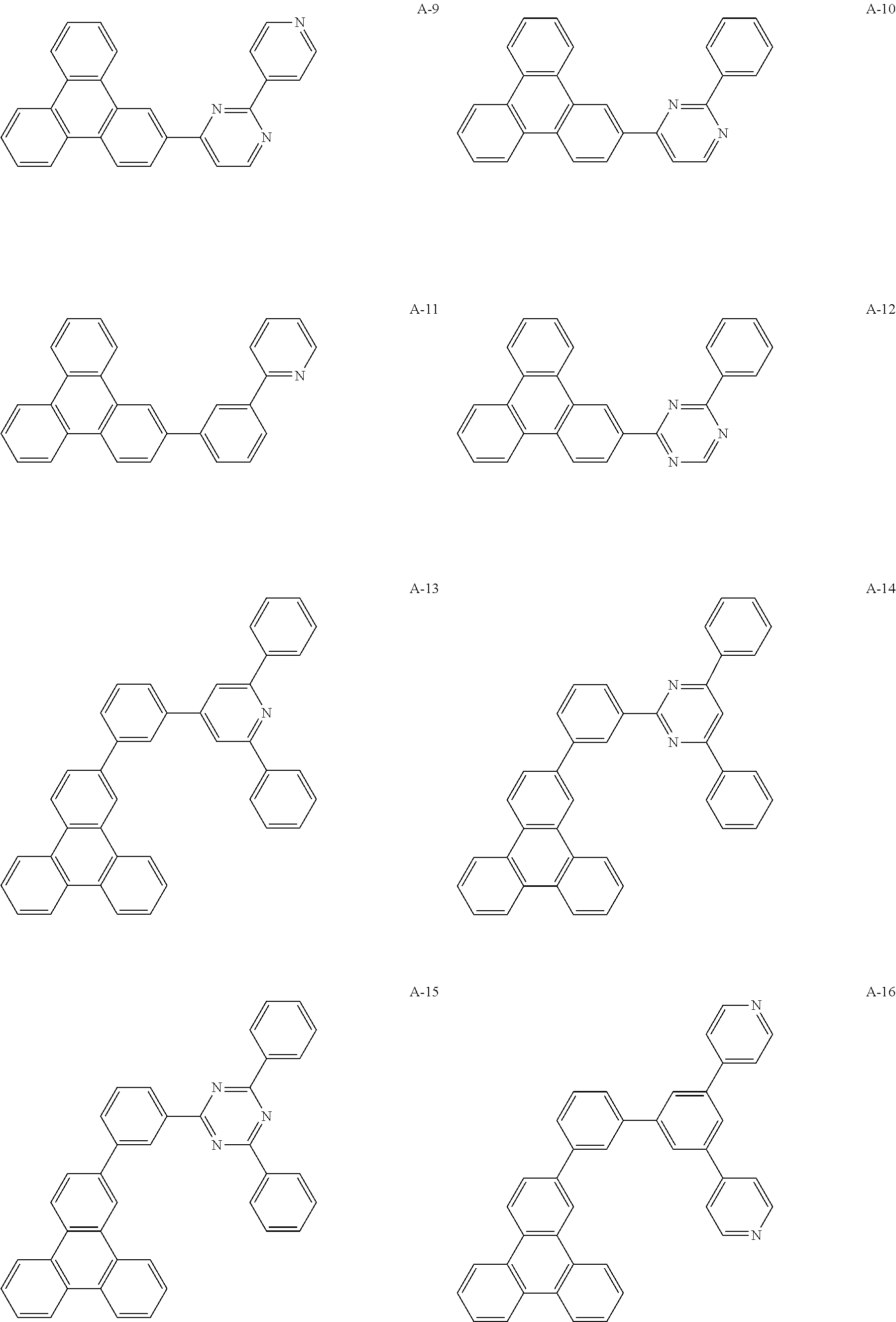

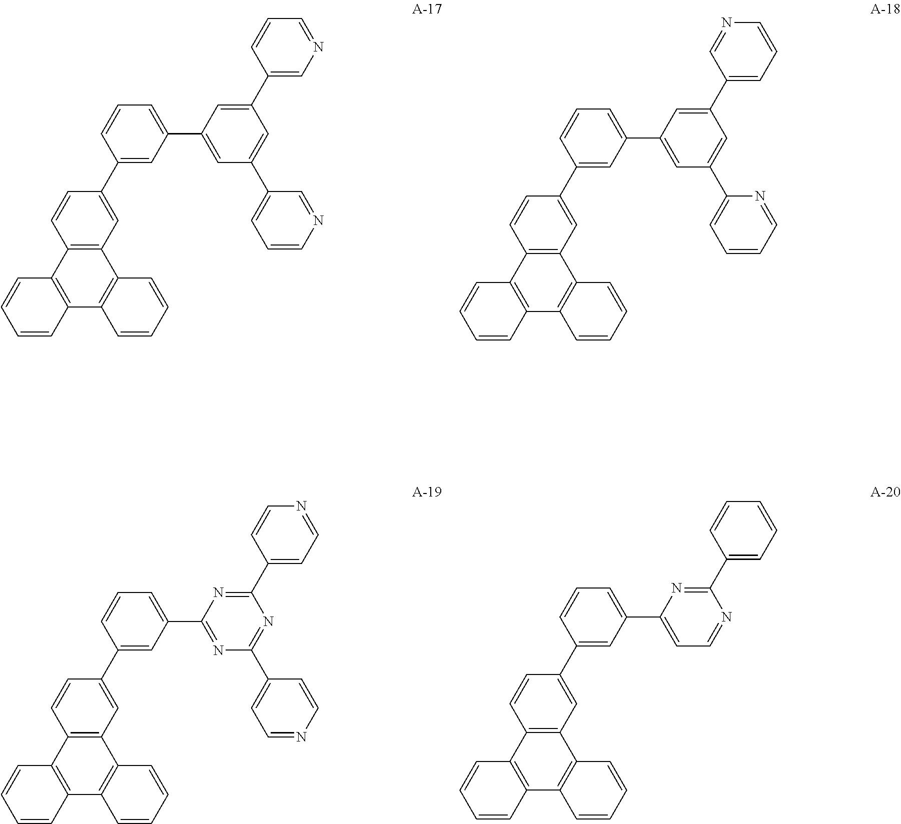 [SMILES: c1cncc(-c2cc(-c3cccnc3)cc(-c3cccc(-c4ccc5c6ccccc6c6ccccc6c5c4)c3)c2)c1 ; c1ccc(-c2cc(-c3cccnc3)cc(-c3cccc(-c4ccc5c6ccccc6c6ccccc6c5c4)c3)c2)nc1 ; c1cc(-c2ccc3c4ccccc4c4ccccc4c3c2)cc(-c2nc(-c3ccncc3)nc(-c3ccncc3)n2)c1 ; c1ccc(-c2nccc(-c3cccc(-c4ccc5c6ccccc6c6ccccc6c5c4)c3)n2)cc1]

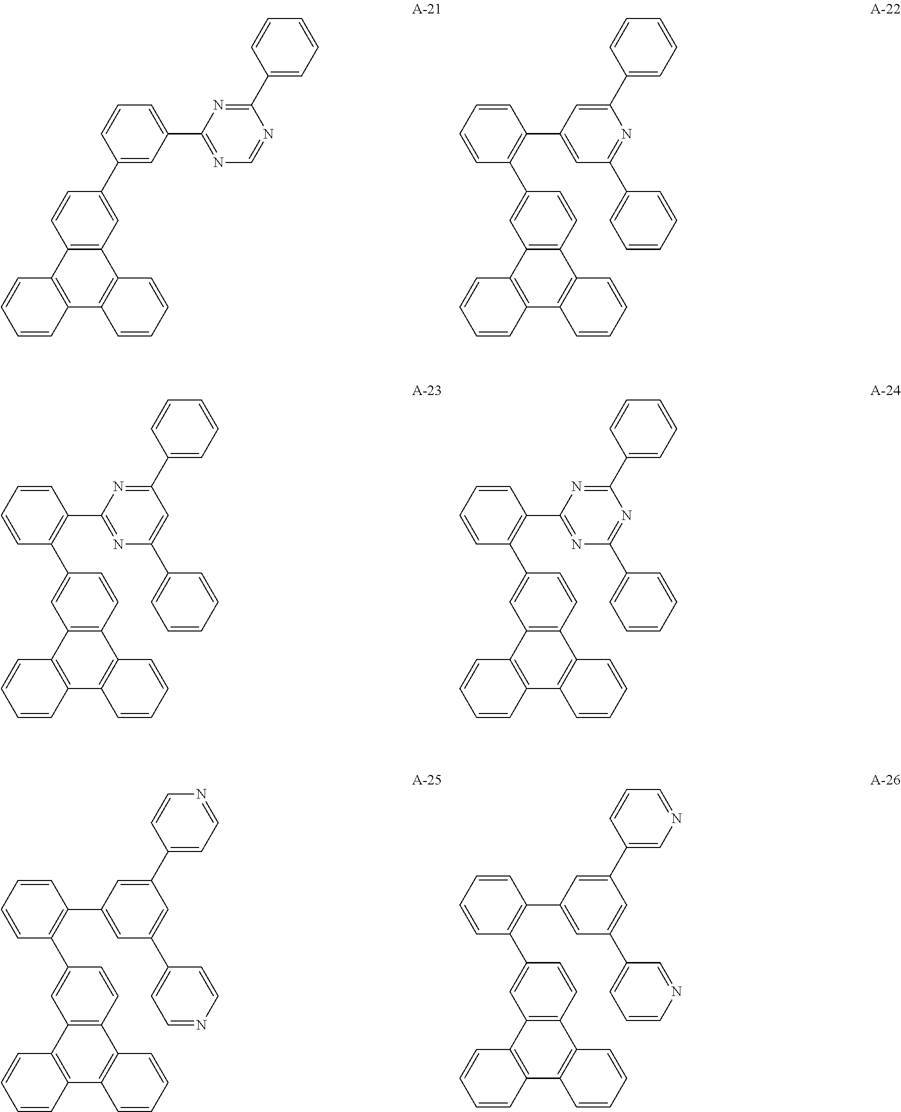 [SMILES: c1ccc(-c2ncnc(-c3cccc(-c4ccc5c6ccccc6c6ccccc6c5c4)c3)n2)cc1 ; c1ccc(-c2cc(-c3ccccc3-c3ccc4c5ccccc5c5ccccc5c4c3)cc(-c3ccccc3)n2)cc1 ; c1ccc(-c2cc(-c3ccccc3)nc(-c3ccccc3-c3ccc4c5ccccc5c5ccccc5c4c3)n2)cc1 ; c1ccc(-c2nc(-c3ccccc3)nc(-c3ccccc3-c3ccc4c5ccccc5c5ccccc5c4c3)n2)cc1 ; c1ccc(-c2ccc3c4ccccc4c4ccccc4c3c2)c(-c2cc(-c3ccncc3)cc(-c3ccncc3)c2)c1 ; c1cncc(-c2cc(-c3cccnc3)cc(-c3ccccc3-c3ccc4c5ccccc5c5ccccc5c4c3)c2)c1]

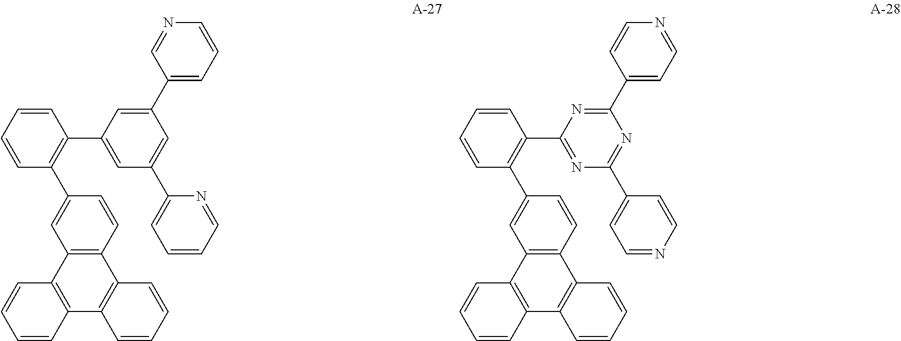 [SMILES: c1ccc(-c2cc(-c3cccnc3)cc(-c3ccccc3-c3ccc4c5ccccc5c5ccccc5c4c3)c2)nc1 ; c1ccc(-c2nc(-c3ccncc3)nc(-c3ccncc3)n2)c(-c2ccc3c4ccccc4c4ccccc4c3c2)c1]

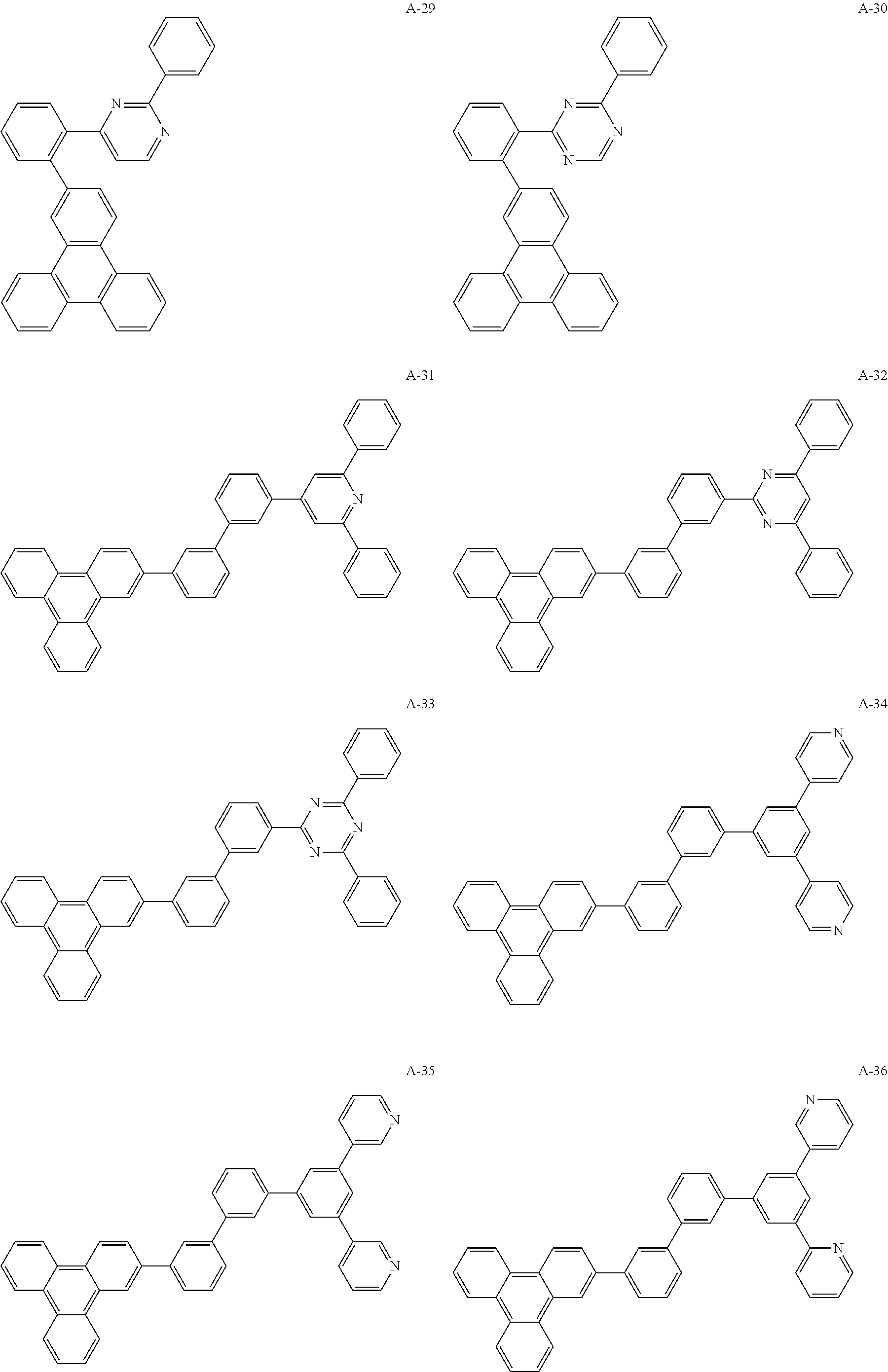

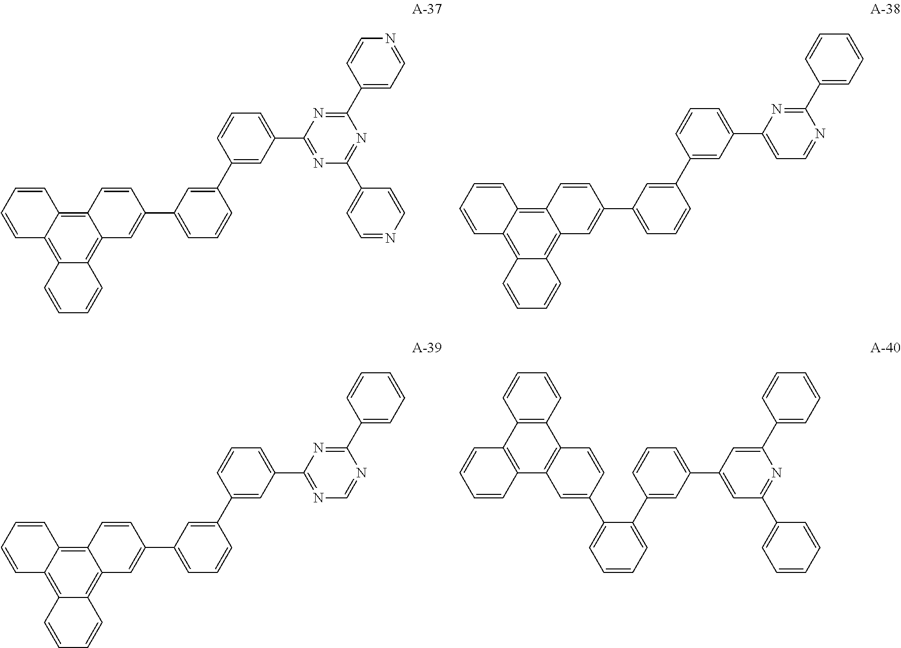 [SMILES: c1cc(-c2cccc(-c3nc(-c4ccncc4)nc(-c4ccncc4)n3)c2)cc(-c2ccc3c4ccccc4c4ccccc4c3c2)c1 ; c1ccc(-c2nccc(-c3cccc(-c4cccc(-c5ccc6c7ccccc7c7ccccc7c6c5)c4)c3)n2)cc1 ; c1ccc(-c2ncnc(-c3cccc(-c4cccc(-c5ccc6c7ccccc7c7ccccc7c6c5)c4)c3)n2)cc1 ; c1ccc(-c2cc(-c3cccc(-c4ccccc4-c4ccc5c6ccccc6c6ccccc6c5c4)c3)cc(-c3ccccc3)n2)cc1]

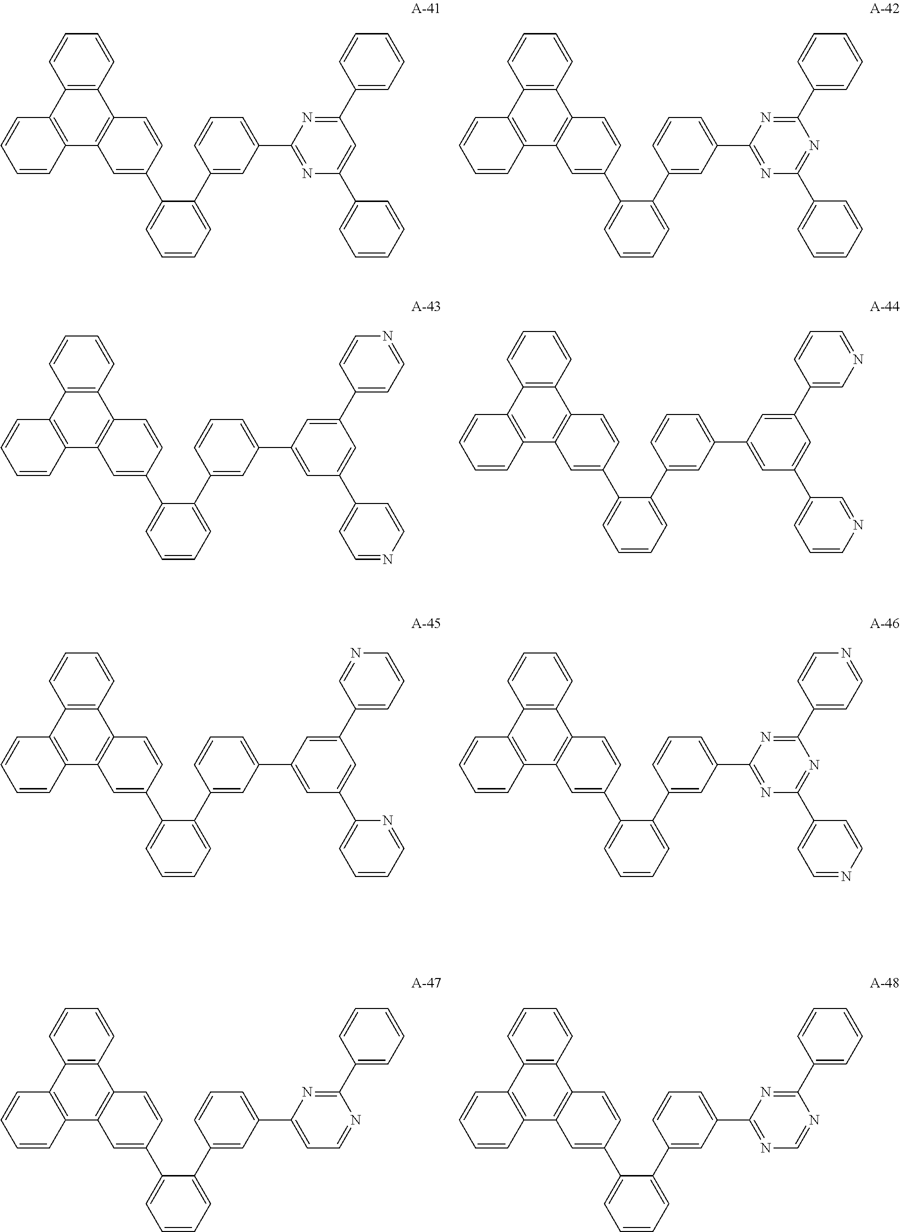

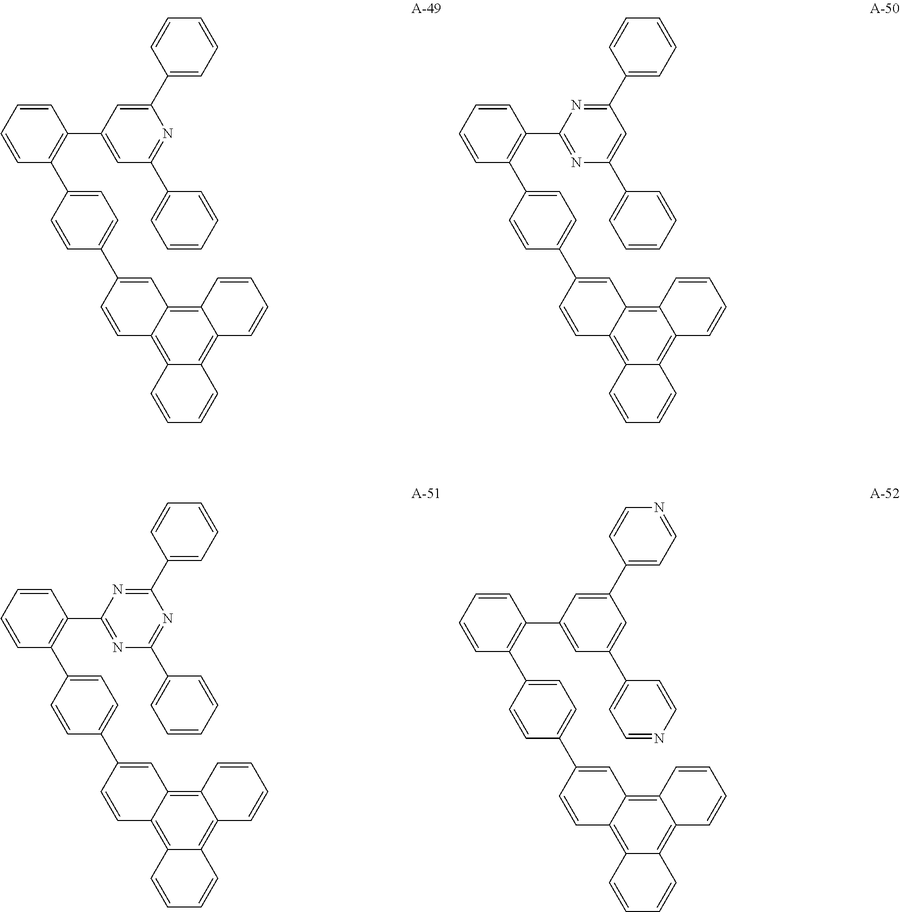 [SMILES: c1ccc(-c2cc(-c3ccccc3-c3ccc(-c4ccc5c6ccccc6c6ccccc6c5c4)cc3)cc(-c3ccccc3)n2)cc1 ; c1ccc(-c2cc(-c3ccccc3)nc(-c3ccccc3-c3ccc(-c4ccc5c6ccccc6c6ccccc6c5c4)cc3)n2)cc1 ; c1ccc(-c2nc(-c3ccccc3)nc(-c3ccccc3-c3ccc(-c4ccc5c6ccccc6c6ccccc6c5c4)cc3)n2)cc1 ; c1ccc(-c2cc(-c3ccncc3)cc(-c3ccncc3)c2)c(-c2ccc(-c3ccc4c5ccccc5c5ccccc5c4c3)cc2)c1]

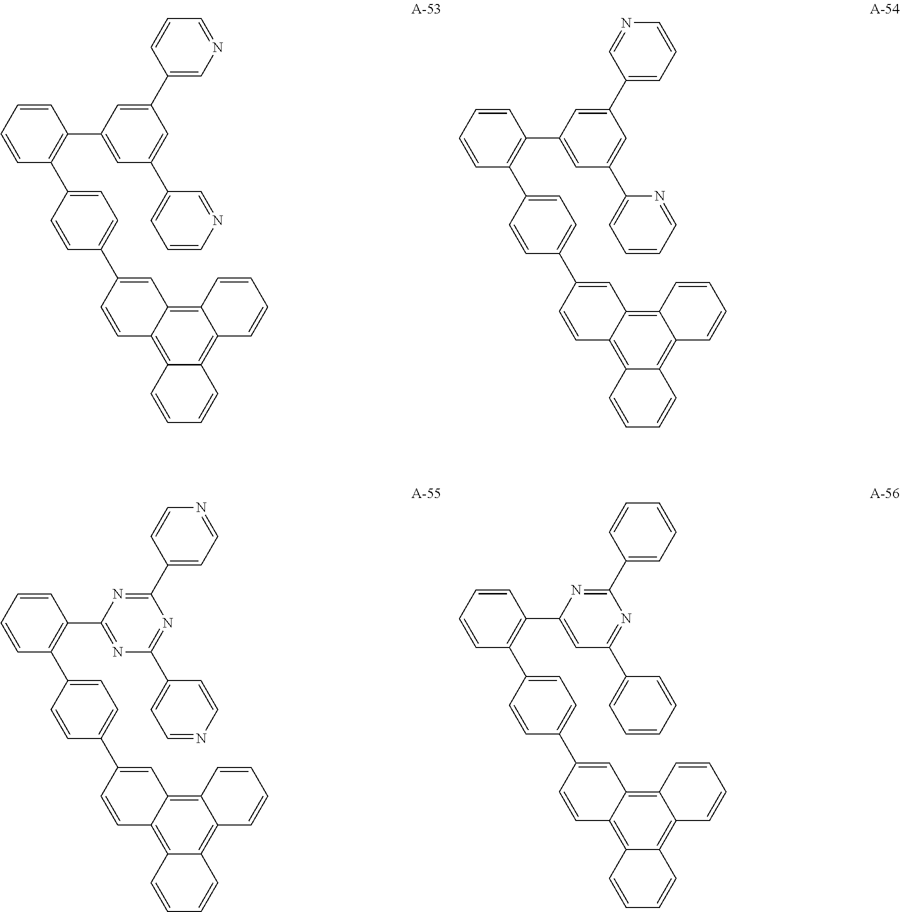 [SMILES: c1cncc(-c2cc(-c3cccnc3)cc(-c3ccccc3-c3ccc(-c4ccc5c6ccccc6c6ccccc6c5c4)cc3)c2)c1 ; c1ccc(-c2cc(-c3cccnc3)cc(-c3ccccc3-c3ccc(-c4ccc5c6ccccc6c6ccccc6c5c4)cc3)c2)nc1 ; c1ccc(-c2nc(-c3ccncc3)nc(-c3ccncc3)n2)c(-c2ccc(-c3ccc4c5ccccc5c5ccccc5c4c3)cc2)c1 ; c1ccc(-c2cc(-c3ccccc3-c3ccc(-c4ccc5c6ccccc6c6ccccc6c5c4)cc3)nc(-c3ccccc3)n2)cc1]

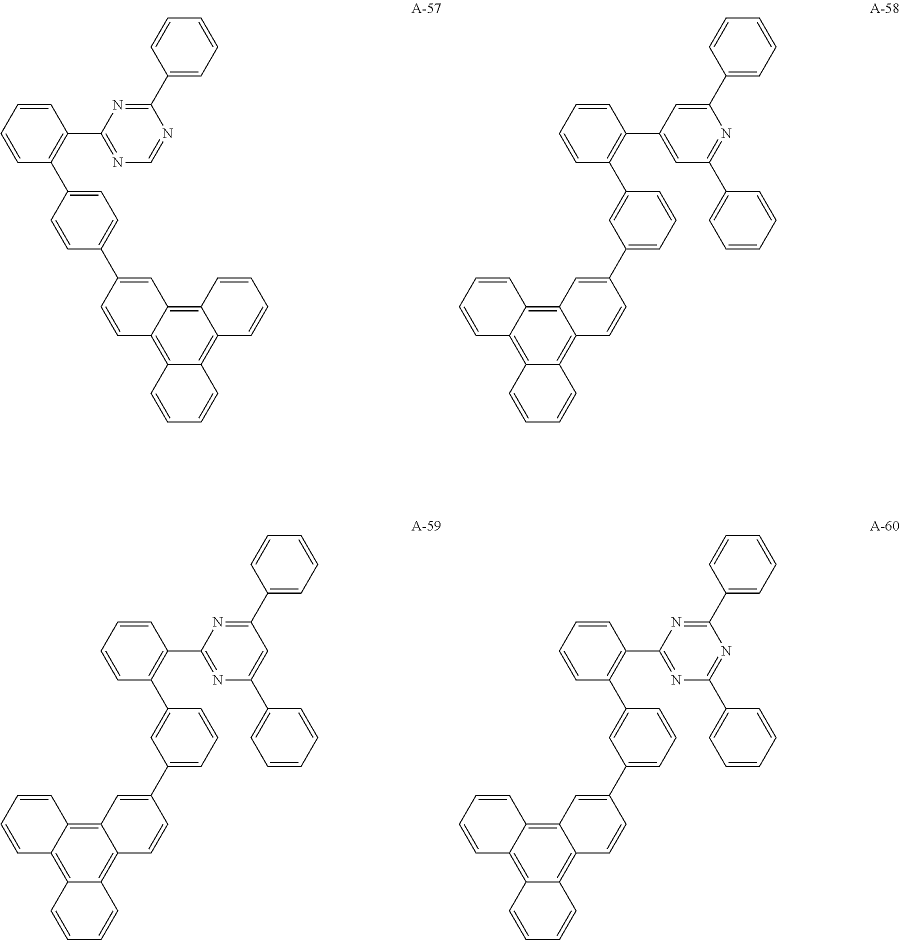 [SMILES: c1ccc(-c2ncnc(-c3ccccc3-c3ccc(-c4ccc5c6ccccc6c6ccccc6c5c4)cc3)n2)cc1 ; c1ccc(-c2cc(-c3ccccc3-c3cccc(-c4ccc5c6ccccc6c6ccccc6c5c4)c3)cc(-c3ccccc3)n2)cc1 ; c1ccc(-c2cc(-c3ccccc3)nc(-c3ccccc3-c3cccc(-c4ccc5c6ccccc6c6ccccc6c5c4)c3)n2)cc1 ; c1ccc(-c2nc(-c3ccccc3)nc(-c3ccccc3-c3cccc(-c4ccc5c6ccccc6c6ccccc6c5c4)c3)n2)cc1]

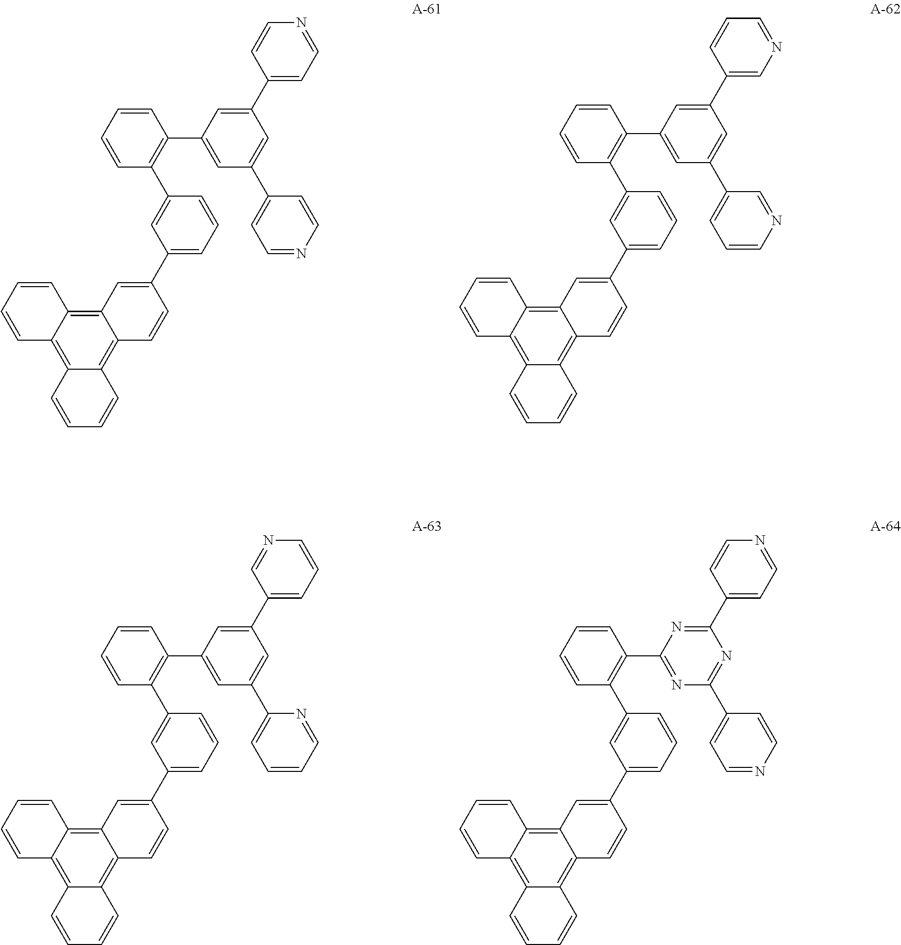 [SMILES: c1cc(-c2ccc3c4ccccc4c4ccccc4c3c2)cc(-c2ccccc2-c2cc(-c3ccncc3)cc(-c3ccncc3)c2)c1 ; c1cncc(-c2cc(-c3cccnc3)cc(-c3ccccc3-c3cccc(-c4ccc5c6ccccc6c6ccccc6c5c4)c3)c2)c1 ; c1ccc(-c2cc(-c3cccnc3)cc(-c3ccccc3-c3cccc(-c4ccc5c6ccccc6c6ccccc6c5c4)c3)c2)nc1 ; c1cc(-c2ccc3c4ccccc4c4ccccc4c3c2)cc(-c2ccccc2-c2nc(-c3ccncc3)nc(-c3ccncc3)n2)c1]

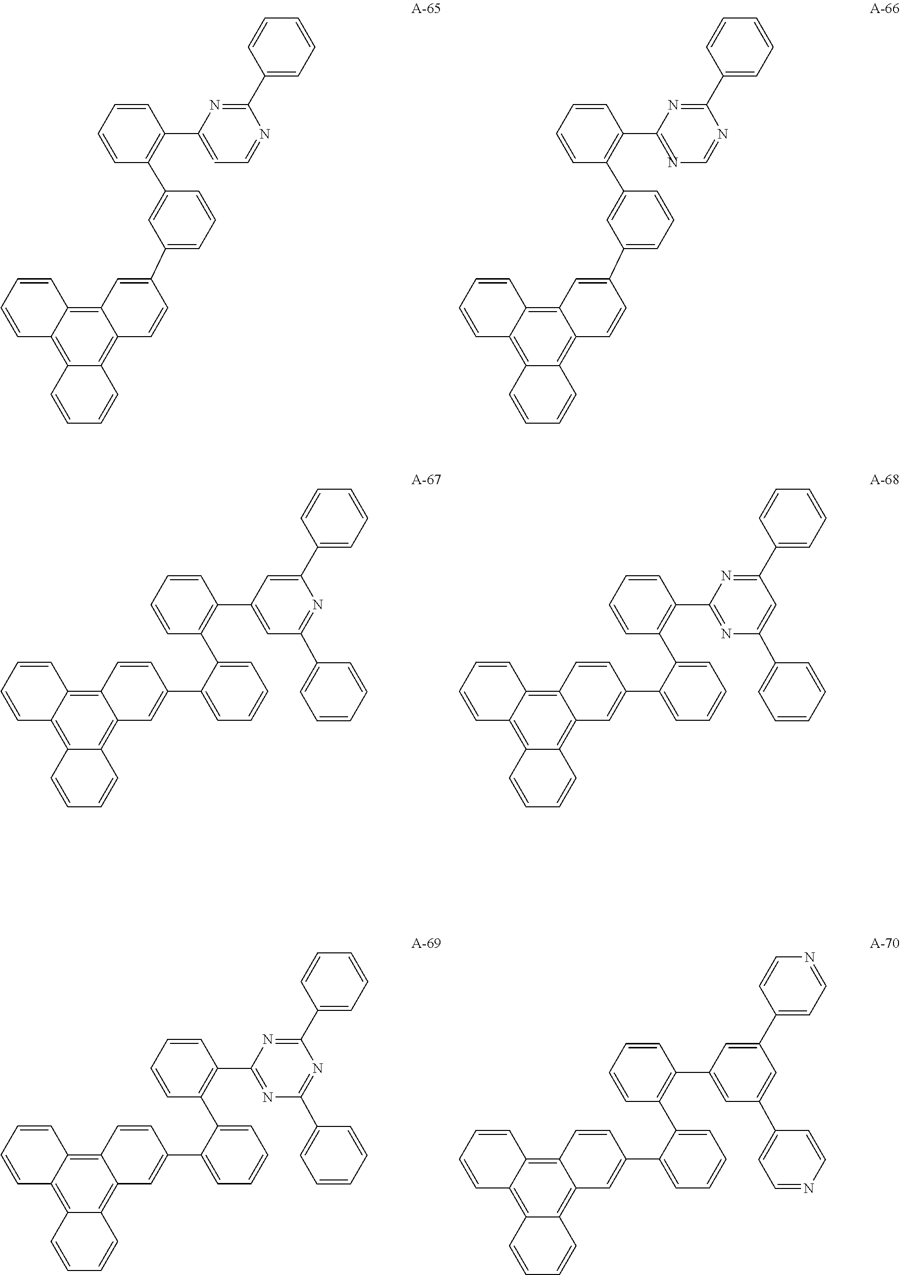 [SMILES: C=C(/N=C(\N=C/C)c1ccccc1)c1ccccc1-c1cccc(-c2ccc3c4ccccc4c4ccccc4c3c2)c1 ; C=C(/N=C(\N=C/N)c1ccccc1)c1ccccc1-c1cccc(-c2ccc3c4ccccc4c4ccccc4c3c2)c1 ; c1ccc(-c2cc(-c3ccccc3-c3ccccc3-c3ccc4c5ccccc5c5ccccc5c4c3)cc(-c3ccccc3)n2)cc1 ; c1ccc(-c2cc(-c3ccccc3)nc(-c3ccccc3-c3ccccc3-c3ccc4c5ccccc5c5ccccc5c4c3)n2)cc1 ; c1ccc(-c2nc(-c3ccccc3)nc(-c3ccccc3-c3ccccc3-c3ccc4c5ccccc5c5ccccc5c4c3)n2)cc1 ; c1ccc(-c2ccccc2-c2ccc3c4ccccc4c4ccccc4c3c2)c(-c2cc(-c3ccncc3)cc(-c3ccncc3)c2)c1]

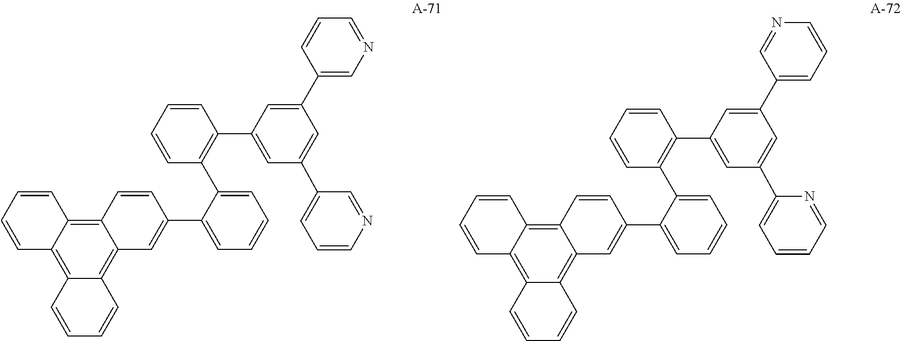 [SMILES: c1cncc(-c2cc(-c3cccnc3)cc(-c3ccccc3-c3ccccc3-c3ccc4c5ccccc5c5ccccc5c4c3)c2)c1 ; c1ccc(-c2cc(-c3cccnc3)cc(-c3ccccc3-c3ccccc3-c3ccc4c5ccccc5c5ccccc5c4c3)c2)nc1]

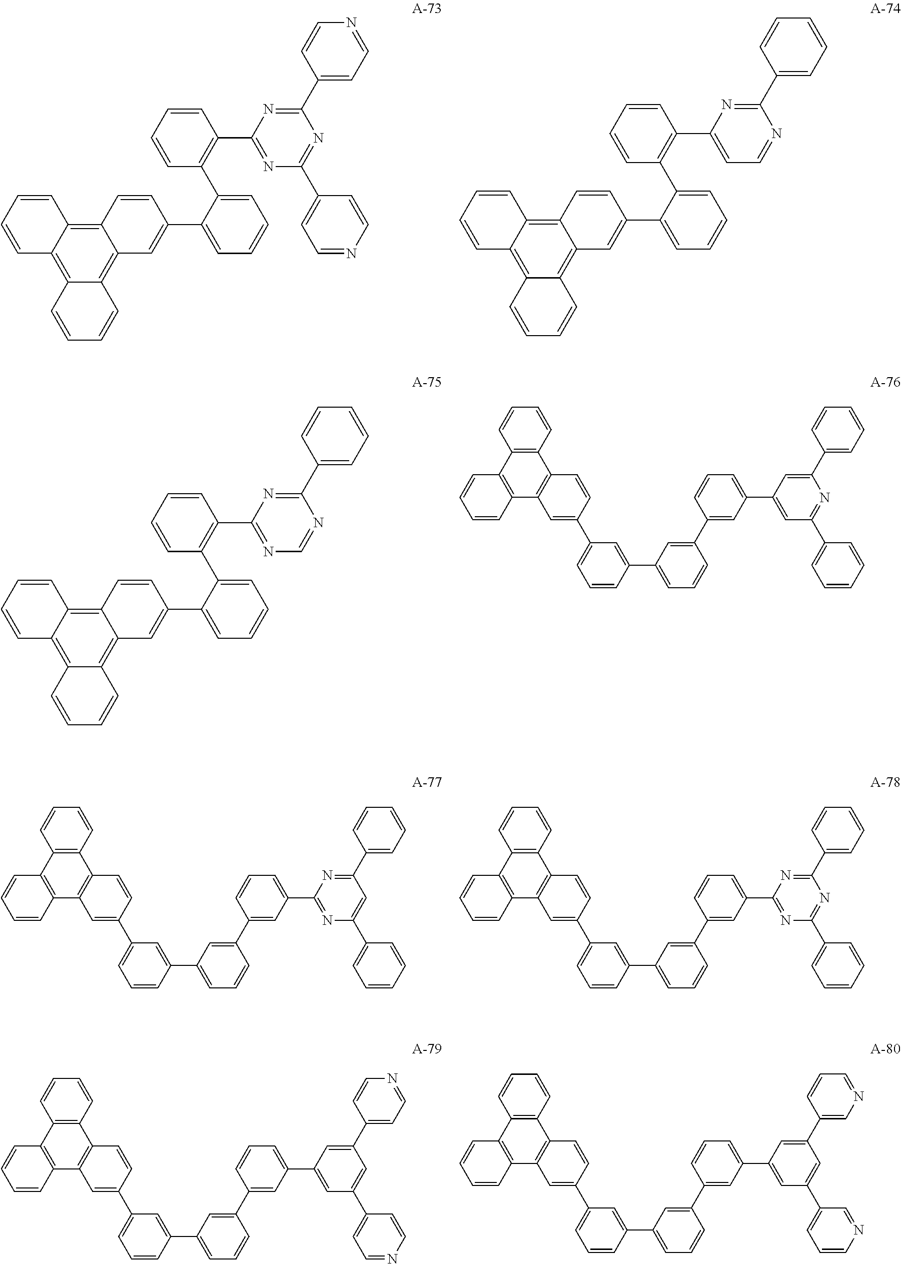

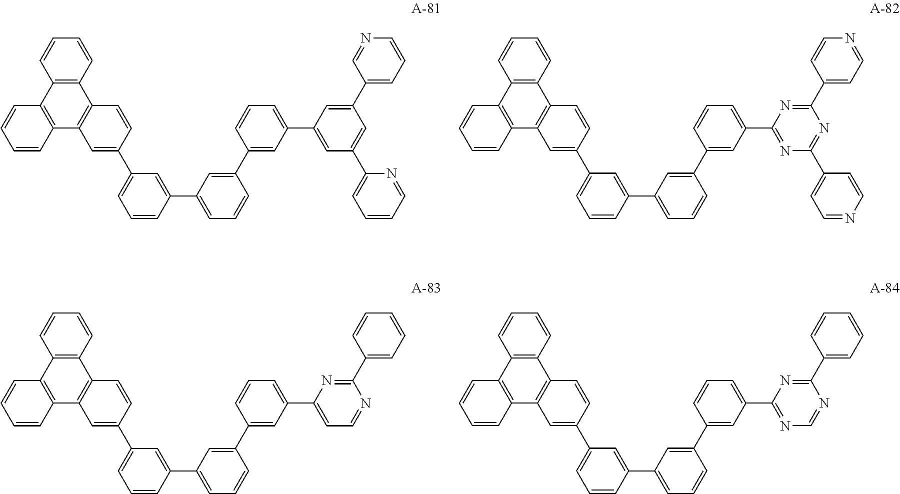 [SMILES: c1ccc(-c2cc(-c3cccnc3)cc(-c3cccc(-c4cccc(-c5cccc(-c6ccc7c8ccccc8c8ccccc8c7c6)c5)c4)c3)c2)nc1 ; c1cc(-c2cccc(-c3ccc4c5ccccc5c5ccccc5c4c3)c2)cc(-c2cccc(-c3nc(-c4ccncc4)nc(-c4ccncc4)n3)c2)c1 ; c1ccc(-c2nccc(-c3cccc(-c4cccc(-c5cccc(-c6ccc7c8ccccc8c8ccccc8c7c6)c5)c4)c3)n2)cc1 ; c1ccc(-c2ncnc(-c3cccc(-c4cccc(-c5cccc(-c6ccc7c8ccccc8c8ccccc8c7c6)c5)c4)c3)n2)cc1]

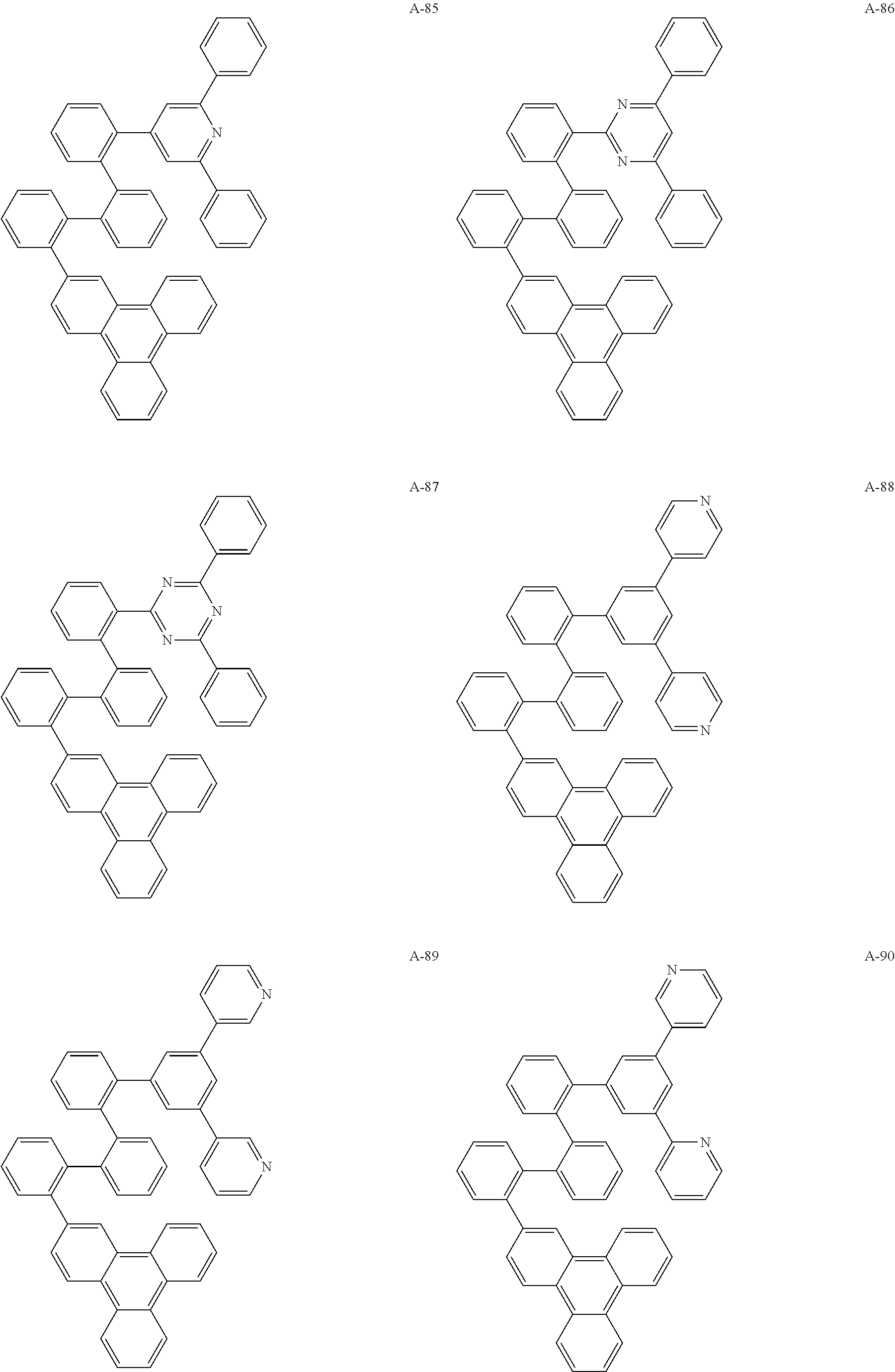

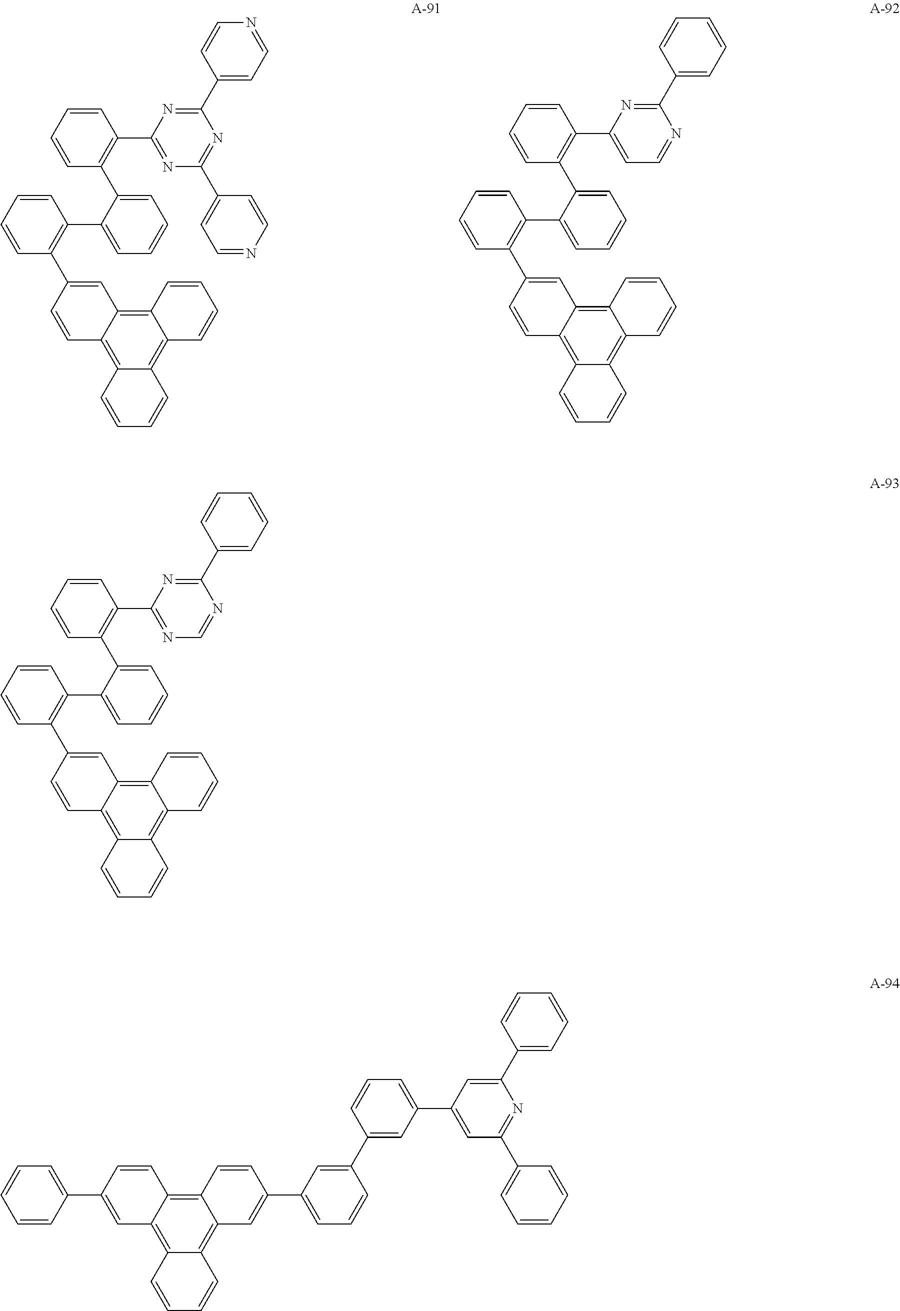 [SMILES: c1ccc(-c2ccccc2-c2ccccc2-c2nc(-c3ccncc3)nc(-c3ccncc3)n2)c(-c2ccc3c4ccccc4c4ccccc4c3c2)c1 ; c1ccc(-c2nccc(-c3ccccc3-c3ccccc3-c3ccccc3-c3ccc4c5ccccc5c5ccccc5c4c3)n2)cc1 ; c1ccc(-c2ncnc(-c3ccccc3-c3ccccc3-c3ccccc3-c3ccc4c5ccccc5c5ccccc5c4c3)n2)cc1 ; c1ccc(-c2ccc3c4ccc(-c5cccc(-c6cccc(-c7cc(-c8ccccc8)nc(-c8ccccc8)c7)c6)c5)cc4c4ccccc4c3c2)cc1]

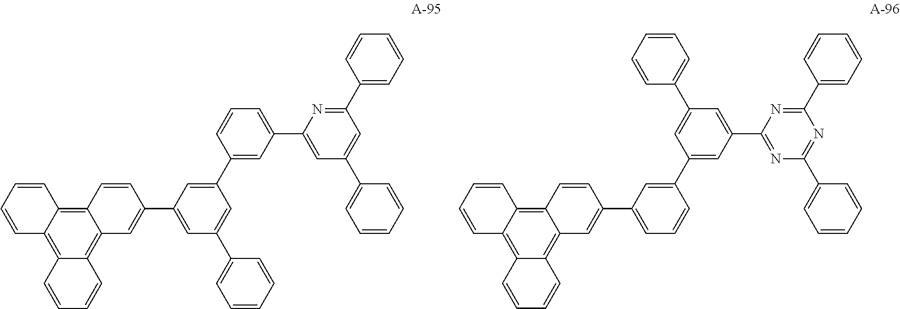 [SMILES: c1ccc(-c2cc(-c3cccc(-c4cc(-c5ccccc5)cc(-c5ccccc5)n4)c3)cc(-c3ccc4c5ccccc5c5ccccc5c4c3)c2)cc1 ; c1ccc(-c2cc(-c3cccc(-c4ccc5c6ccccc6c6ccccc6c5c4)c3)cc(-c3nc(-c4ccccc4)nc(-c4ccccc4)n3)c2)cc1]

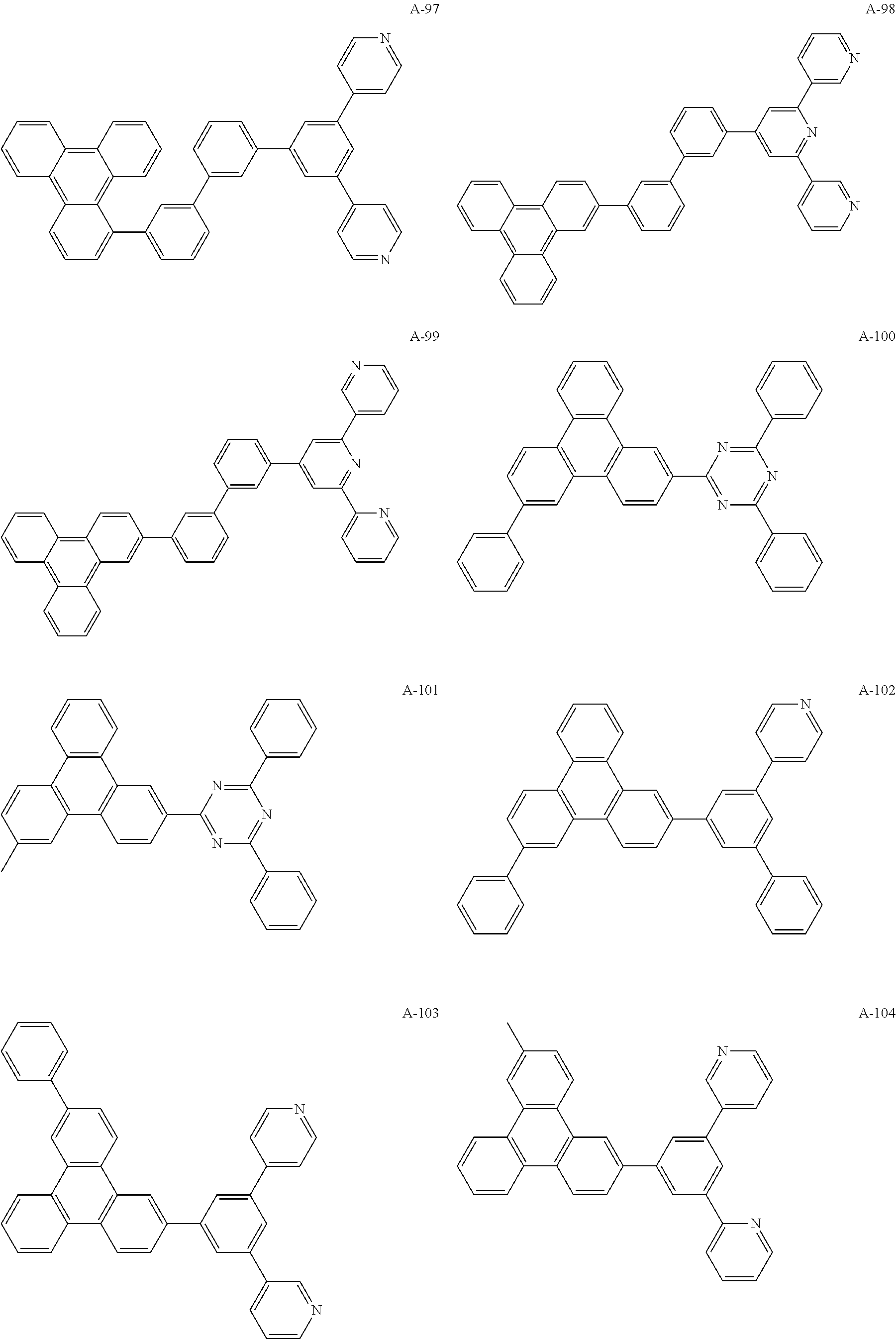

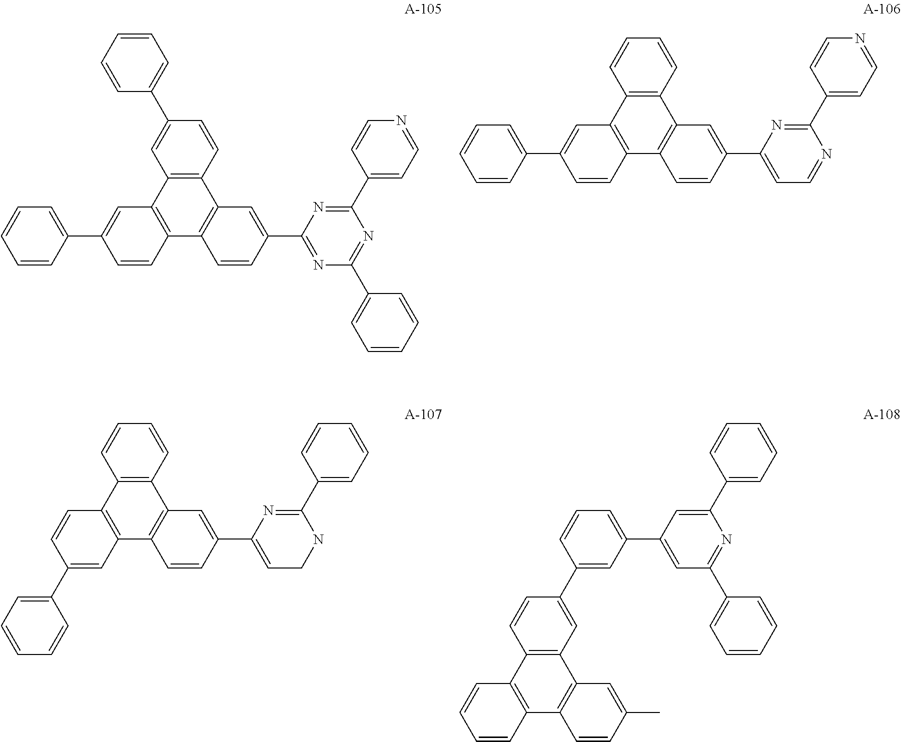

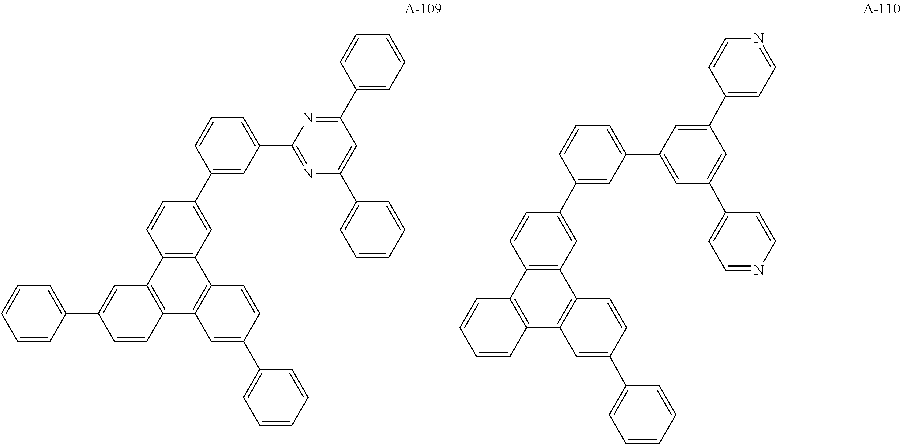 [SMILES: c1ccc(-c2ccc3c(c2)c2ccc(-c4cccc(-c5nc(-c6ccccc6)cc(-c6ccccc6)n5)c4)cc2c2ccc(-c4ccccc4)cc32)cc1 ; c1ccc(-c2ccc3c(c2)c2ccccc2c2ccc(-c4cccc(-c5cc(-c6ccncc6)cc(-c6ccncc6)c5)c4)cc23)cc1]

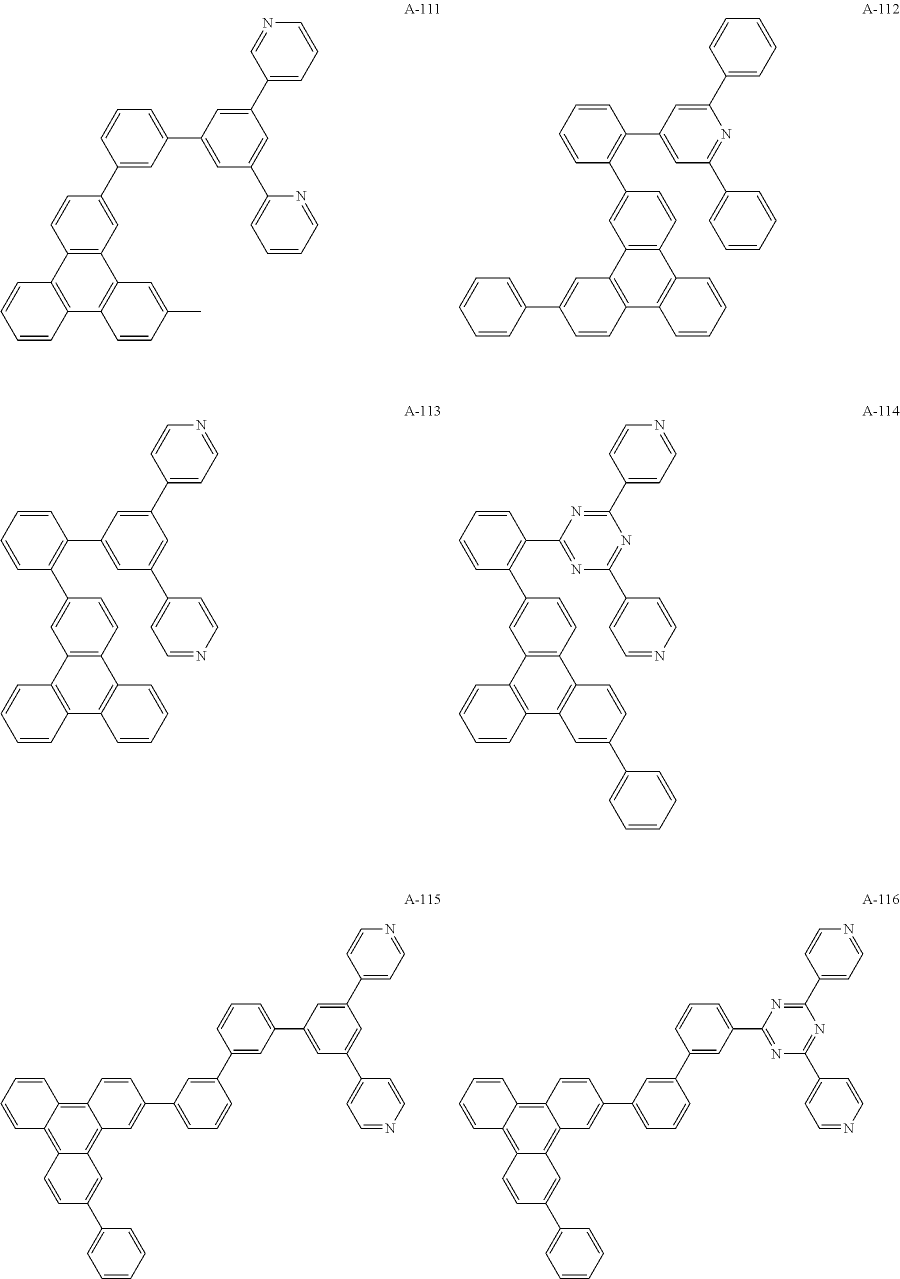 [SMILES: Cc1ccc2c3ccccc3c3ccc(-c4cccc(-c5cc(-c6cccnc6)cc(-c6ccccn6)c5)c4)cc3c2c1 ; c1ccc(-c2ccc3c4ccccc4c4ccc(-c5ccccc5-c5cc(-c6ccccc6)nc(-c6ccccc6)c5)cc4c3c2)cc1 ; c1ccc(-c2ccc3c4ccccc4c4ccccc4c3c2)c(-c2cc(-c3ccncc3)cc(-c3ccncc3)c2)c1 ; c1ccc(-c2ccc3c4ccc(-c5ccccc5-c5nc(-c6ccncc6)nc(-c6ccncc6)n5)cc4c4ccccc4c3c2)cc1 ; c1ccc(-c2ccc3c4ccccc4c4ccc(-c5cccc(-c6cccc(-c7cc(-c8ccncc8)cc(-c8ccncc8)c7)c6)c5)cc4c3c2)cc1 ; c1ccc(-c2ccc3c4ccccc4c4ccc(-c5cccc(-c6cccc(-c7nc(-c8ccncc8)nc(-c8ccncc8)n7)c6)c5)cc4c3c2)cc1]

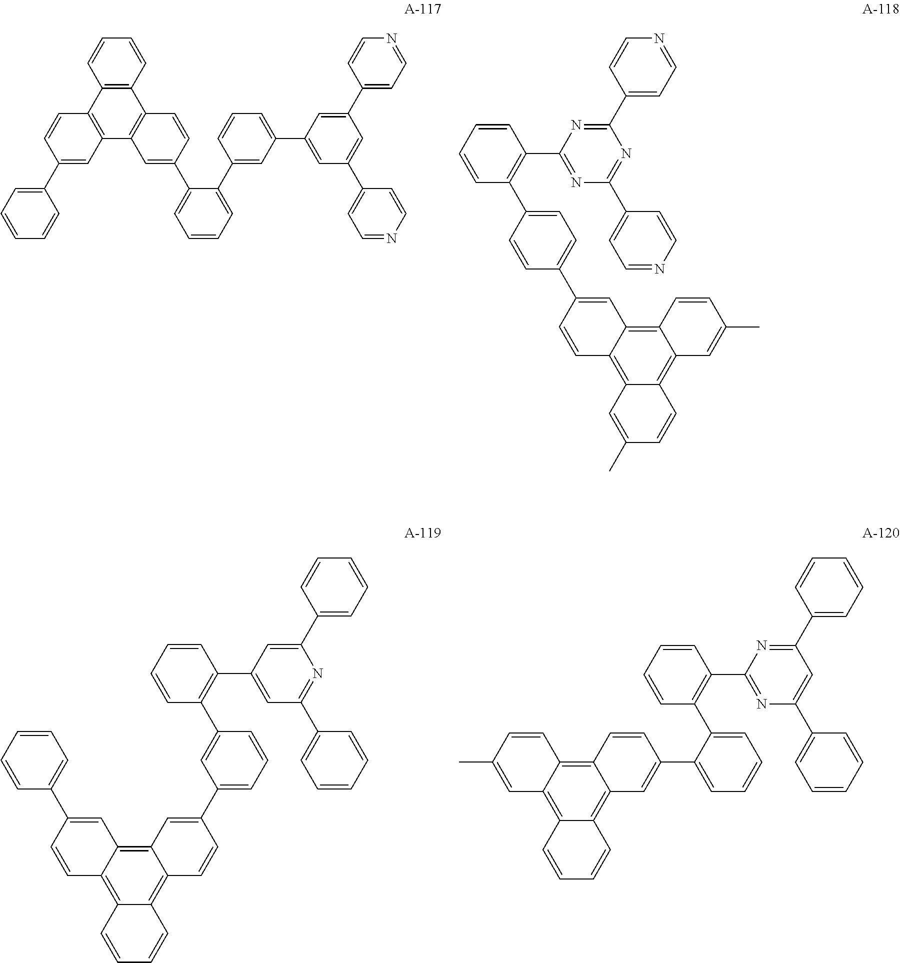 [SMILES: c1ccc(-c2ccc3c4ccccc4c4ccc(-c5ccccc5-c5cccc(-c6cc(-c7ccncc7)cc(-c7ccncc7)c6)c5)cc4c3c2)cc1 ; Cc1ccc2c(c1)c1ccc(C)cc1c1ccc(-c3ccc(-c4ccccc4-c4nc(-c5ccncc5)nc(-c5ccncc5)n4)cc3)cc21 ; c1ccc(-c2ccc3c4ccccc4c4ccc(-c5cccc(-c6ccccc6-c6cc(-c7ccccc7)nc(-c7ccccc7)c6)c5)cc4c3c2)cc1 ; Cc1ccc2c3ccc(-c4ccccc4-c4ccccc4-c4nc(-c5ccccc5)cc(-c5ccccc5)n4)cc3c3ccccc3c2c1]

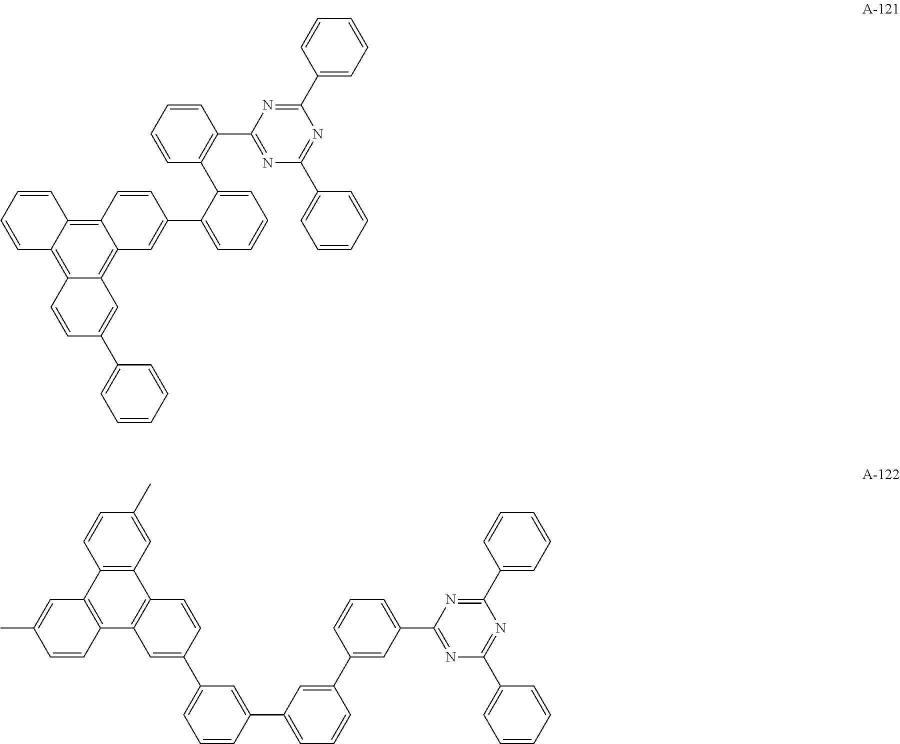 [SMILES: c1ccc(-c2ccc3c4ccccc4c4ccc(-c5ccccc5-c5ccccc5-c5nc(-c6ccccc6)nc(-c6ccccc6)n5)cc4c3c2)cc1 ; Cc1ccc2c(c1)c1ccc(C)cc1c1ccc(-c3cccc(-c4cccc(-c5cccc(-c6nc(-c7ccccc7)nc(-c7ccccc7)n6)c5)c4)c3)cc21]

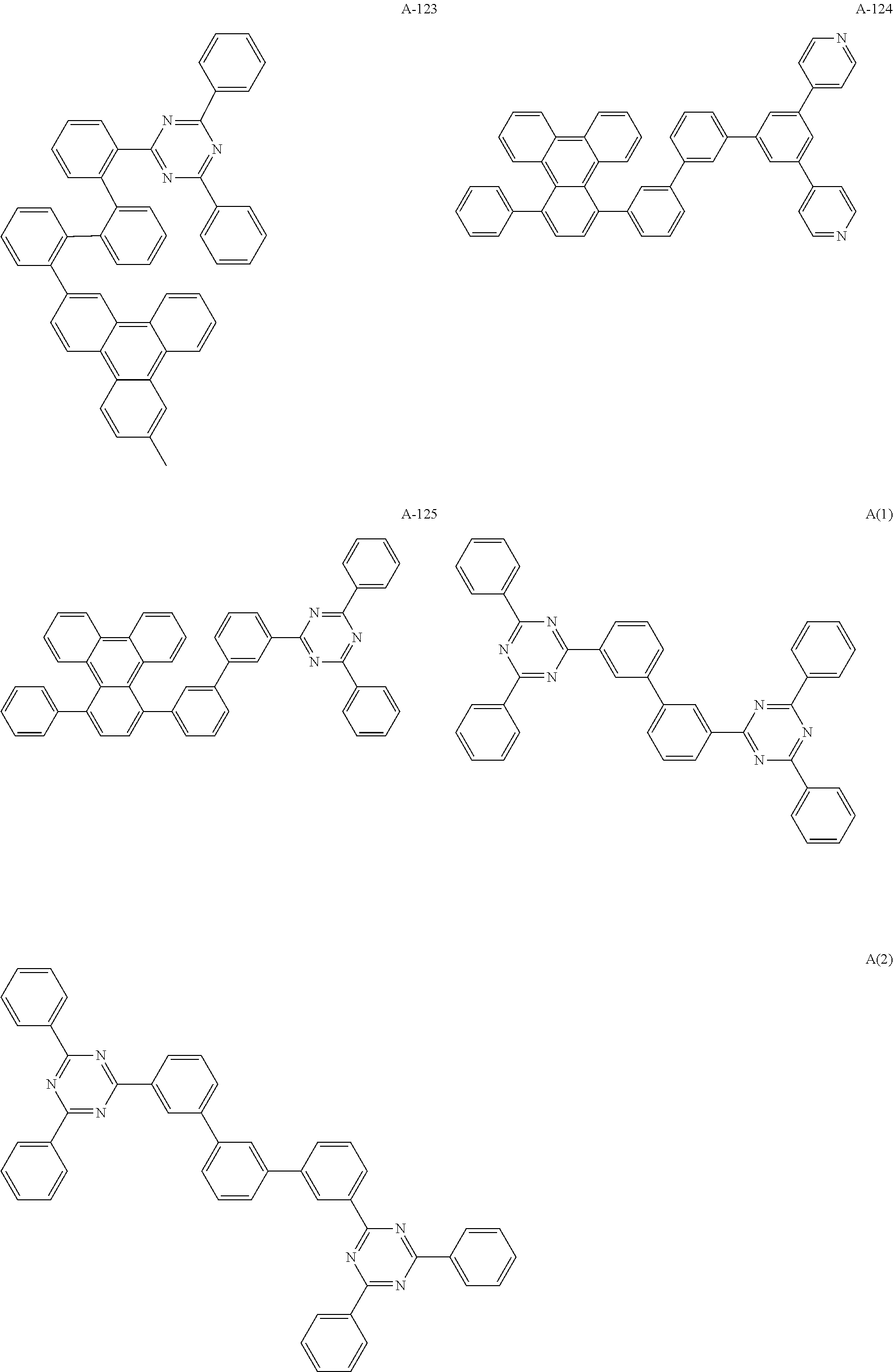 [SMILES: Cc1ccc2c3ccc(-c4ccccc4-c4ccccc4-c4ccccc4-c4nc(-c5ccccc5)nc(-c5ccccc5)n4)cc3c3ccccc3c2c1 ; c1ccc(-c2ccc(-c3cccc(-c4cccc(-c5cc(-c6ccncc6)cc(-c6ccncc6)c5)c4)c3)c3c4ccccc4c4ccccc4c23)cc1 ; c1ccc(-c2nc(-c3ccccc3)nc(-c3cccc(-c4cccc(-c5ccc(-c6ccccc6)c6c7ccccc7c7ccccc7c56)c4)c3)n2)cc1 ; c1ccc(-c2nc(-c3ccccc3)nc(-c3cccc(-c4cccc(-c5nc(-c6ccccc6)nc(-c6ccccc6)n5)c4)c3)n2)cc1 ; c1ccc(-c2nc(-c3ccccc3)nc(-c3cccc(-c4cccc(-c5cccc(-c6nc(-c7ccccc7)nc(-c7ccccc7)n6)c5)c4)c3)n2)cc1]

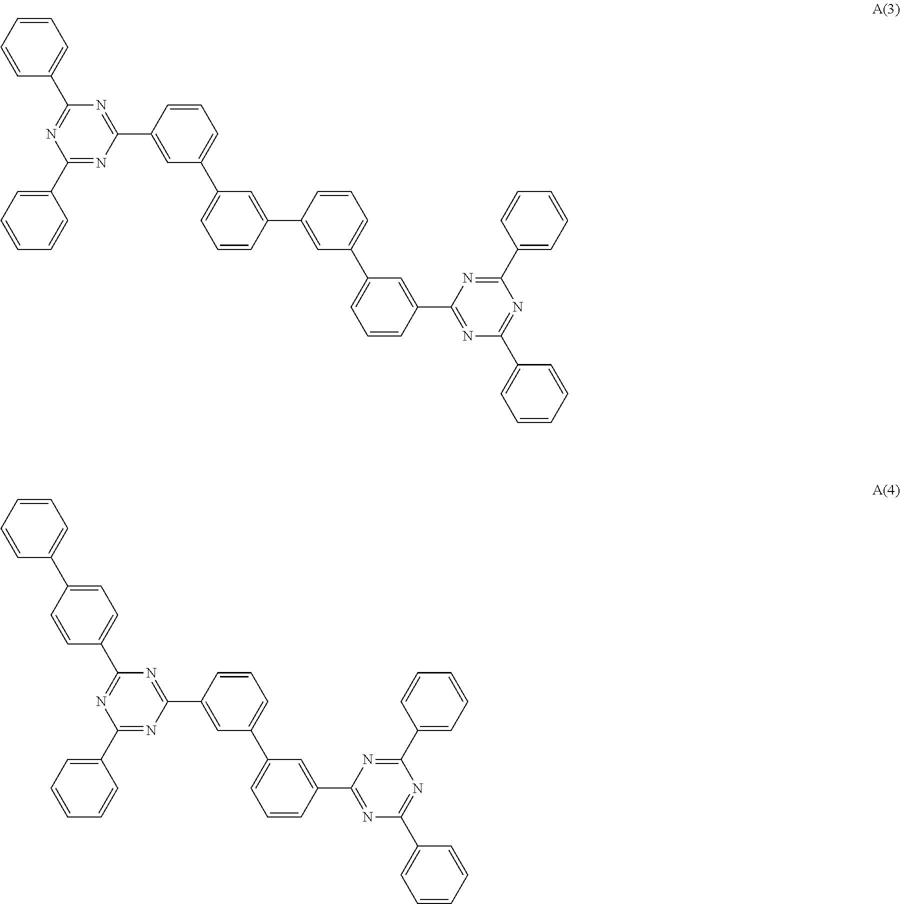 [SMILES: c1ccc(-c2nc(-c3ccccc3)nc(-c3cccc(-c4cccc(-c5cccc(-c6cccc(-c7nc(-c8ccccc8)nc(-c8ccccc8)n7)c6)c5)c4)c3)n2)cc1 ; c1ccc(-c2ccc(-c3nc(-c4ccccc4)nc(-c4cccc(-c5cccc(-c6nc(-c7ccccc7)nc(-c7ccccc7)n6)c5)c4)n3)cc2)cc1]

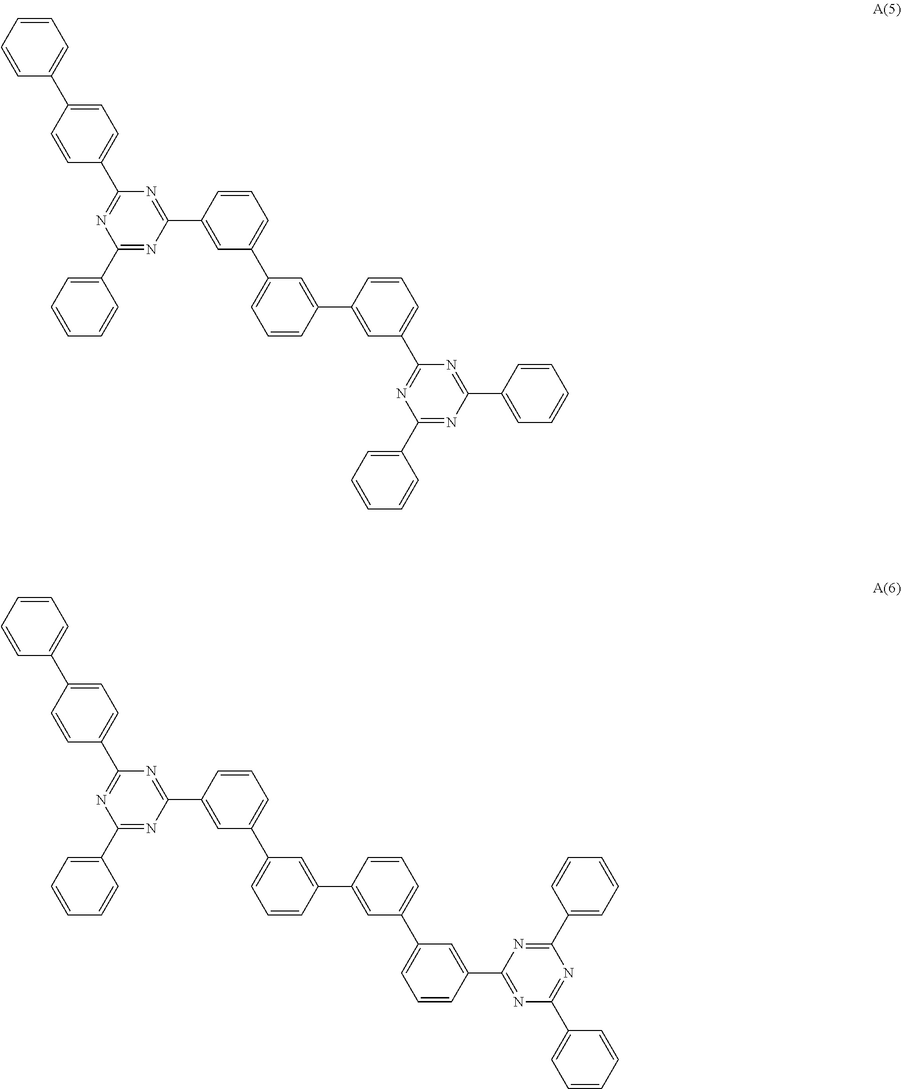 [SMILES: c1ccc(-c2ccc(-c3nc(-c4ccccc4)nc(-c4cccc(-c5cccc(-c6cccc(-c7nc(-c8ccccc8)nc(-c8ccccc8)n7)c6)c5)c4)n3)cc2)cc1 ; c1ccc(-c2ccc(-c3nc(-c4ccccc4)nc(-c4cccc(-c5cccc(-c6cccc(-c7cccc(-c8nc(-c9ccccc9)nc(-c9ccccc9)n8)c7)c6)c5)c4)n3)cc2)cc1]

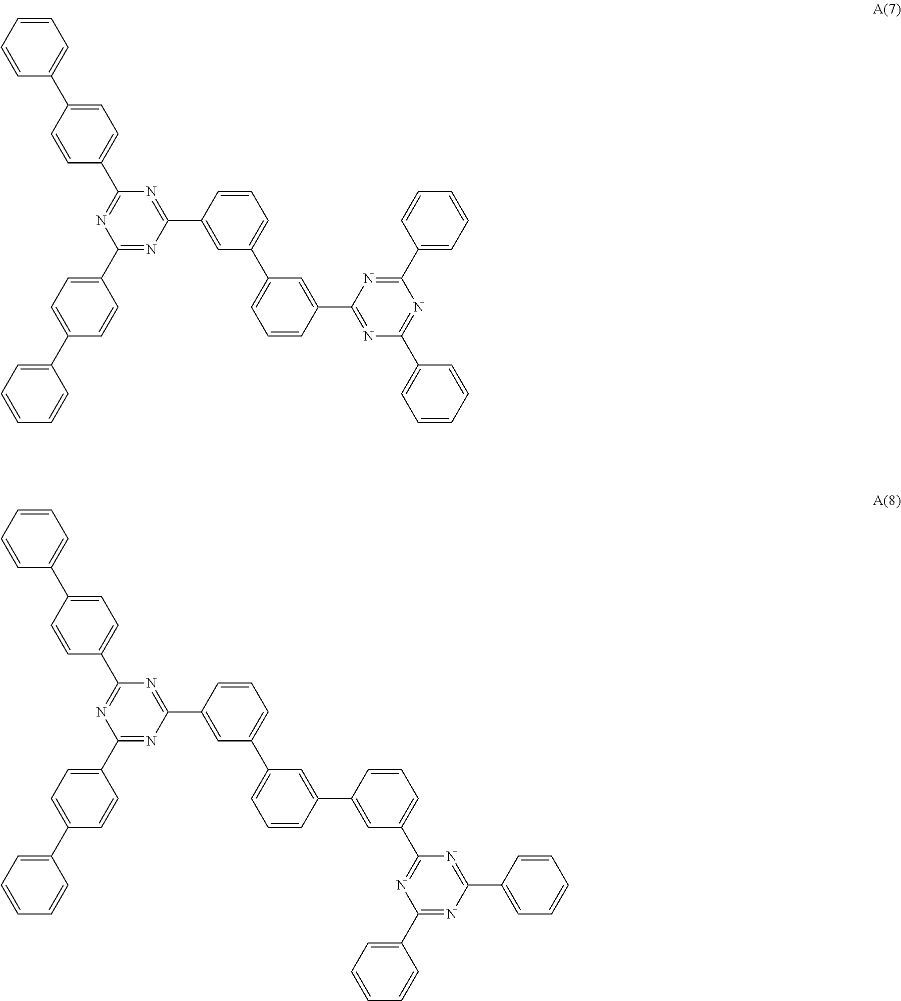 [SMILES: c1ccc(-c2ccc(-c3nc(-c4ccc(-c5ccccc5)cc4)nc(-c4cccc(-c5cccc(-c6nc(-c7ccccc7)nc(-c7ccccc7)n6)c5)c4)n3)cc2)cc1 ; c1ccc(-c2ccc(-c3nc(-c4ccc(-c5ccccc5)cc4)nc(-c4cccc(-c5cccc(-c6cccc(-c7nc(-c8ccccc8)nc(-c8ccccc8)n7)c6)c5)c4)n3)cc2)cc1]

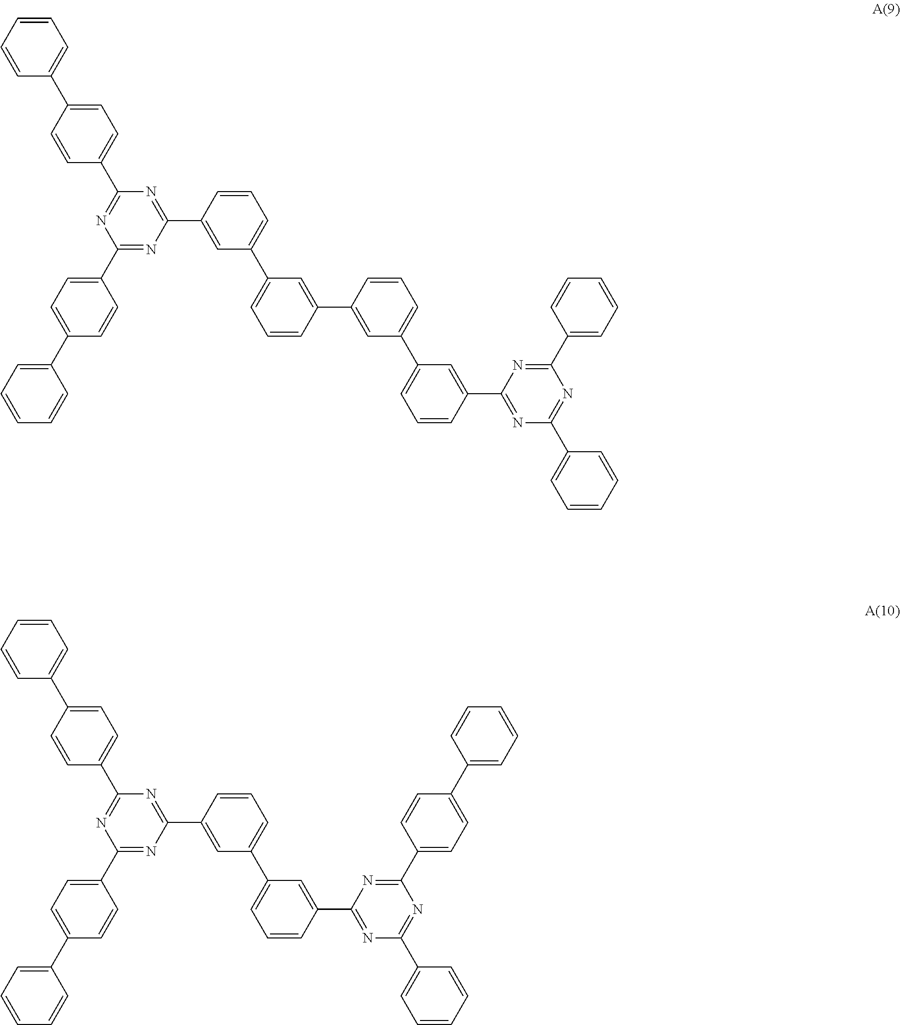 [SMILES: c1ccc(-c2ccc(-c3nc(-c4ccc(-c5ccccc5)cc4)nc(-c4cccc(-c5cccc(-c6cccc(-c7cccc(-c8nc(-c9ccccc9)nc(-c9ccccc9)n8)c7)c6)c5)c4)n3)cc2)cc1 ; c1ccc(-c2ccc(-c3nc(-c4ccccc4)nc(-c4cccc(-c5cccc(-c6nc(-c7ccc(-c8ccccc8)cc7)nc(-c7ccc(-c8ccccc8)cc7)n6)c5)c4)n3)cc2)cc1]

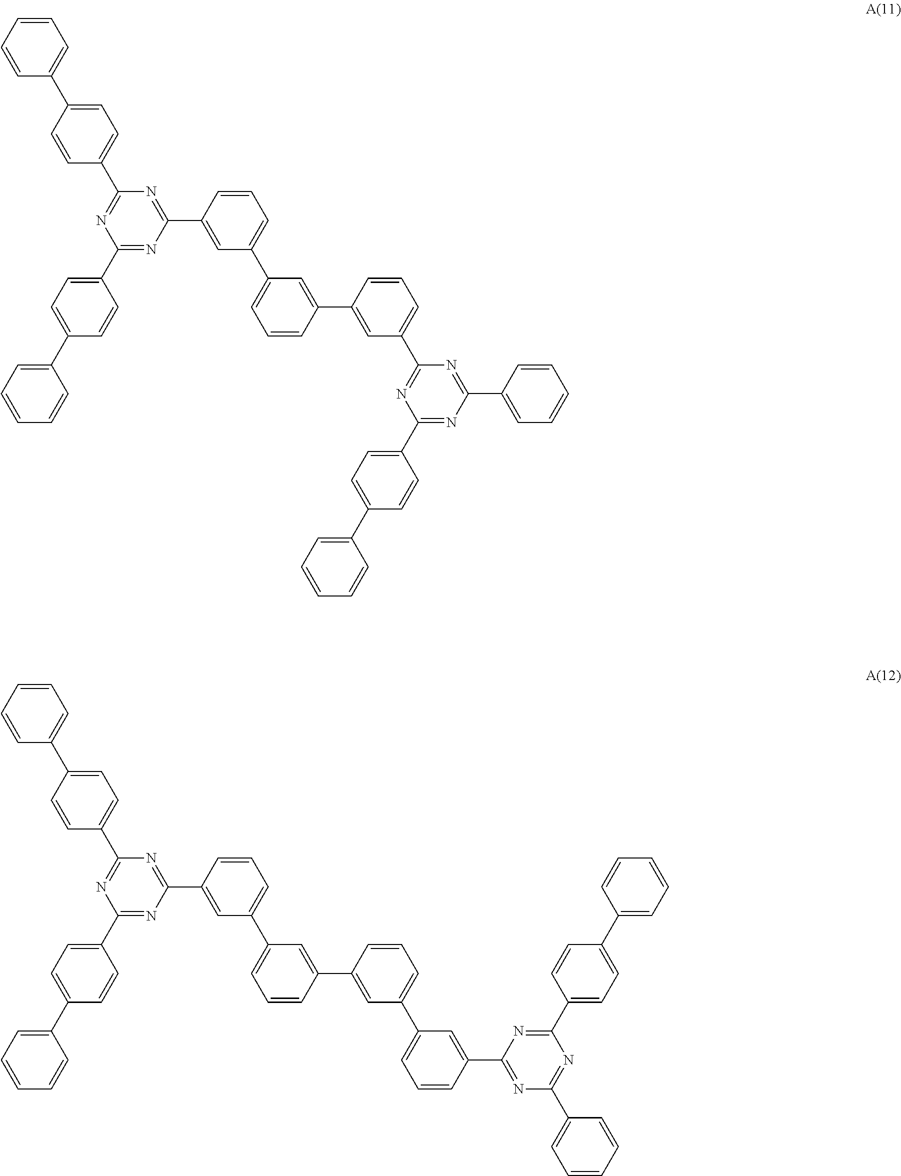 [SMILES: c1ccc(-c2ccc(-c3nc(-c4ccccc4)nc(-c4cccc(-c5cccc(-c6cccc(-c7nc(-c8ccc(-c9ccccc9)cc8)nc(-c8ccc(-c9ccccc9)cc8)n7)c6)c5)c4)n3)cc2)cc1 ; c1ccc(-c2ccc(-c3nc(-c4ccccc4)nc(-c4cccc(-c5cccc(-c6cccc(-c7cccc(-c8nc(-c9ccc(-c%10ccccc%10)cc9)nc(-c9ccc(-c%10ccccc%10)cc9)n8)c7)c6)c5)c4)n3)cc2)cc1]

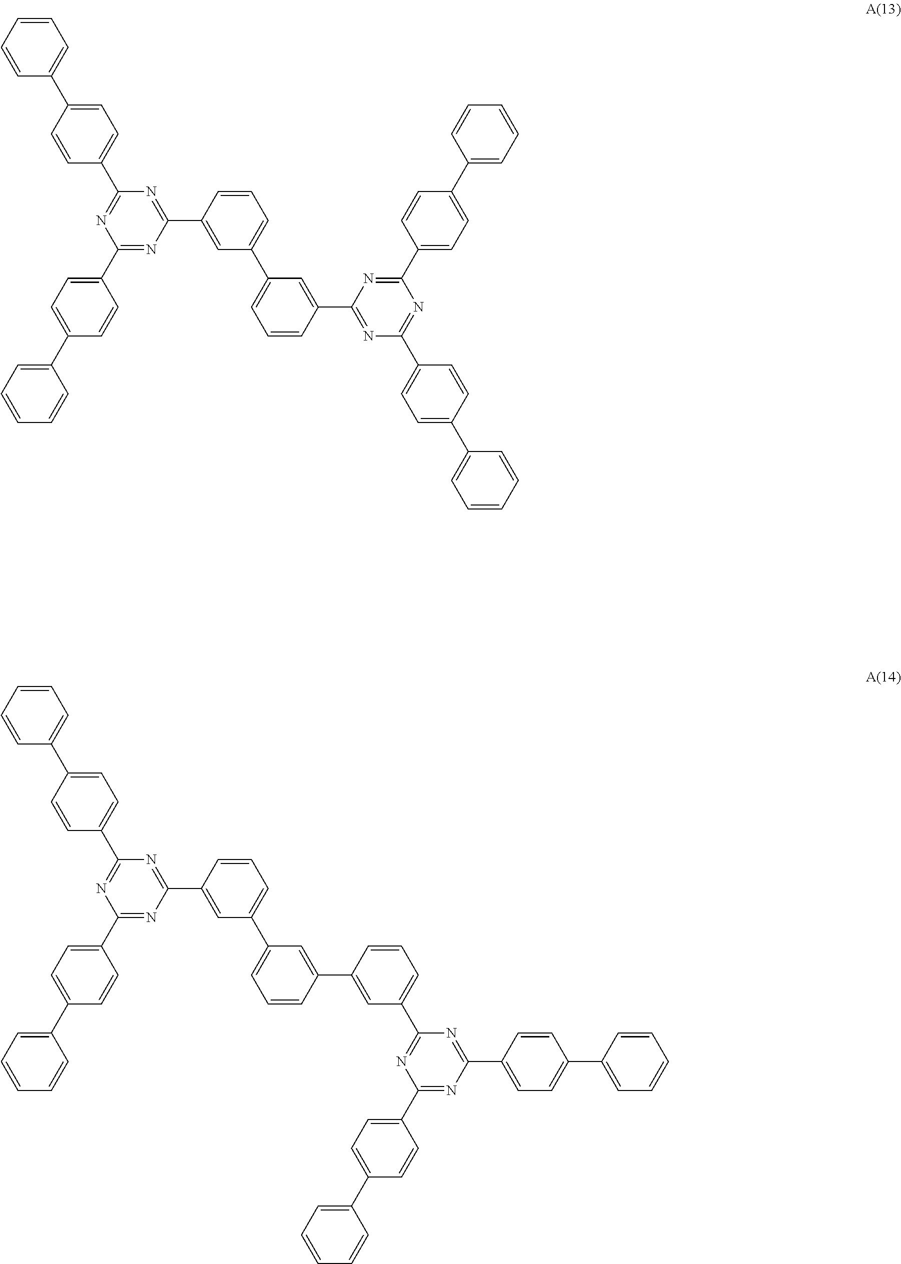 [SMILES: c1ccc(-c2ccc(-c3nc(-c4ccc(-c5ccccc5)cc4)nc(-c4cccc(-c5cccc(-c6nc(-c7ccc(-c8ccccc8)cc7)nc(-c7ccc(-c8ccccc8)cc7)n6)c5)c4)n3)cc2)cc1 ; c1ccc(-c2ccc(-c3nc(-c4ccc(-c5ccccc5)cc4)nc(-c4cccc(-c5cccc(-c6cccc(-c7nc(-c8ccc(-c9ccccc9)cc8)nc(-c8ccc(-c9ccccc9)cc8)n7)c6)c5)c4)n3)cc2)cc1]

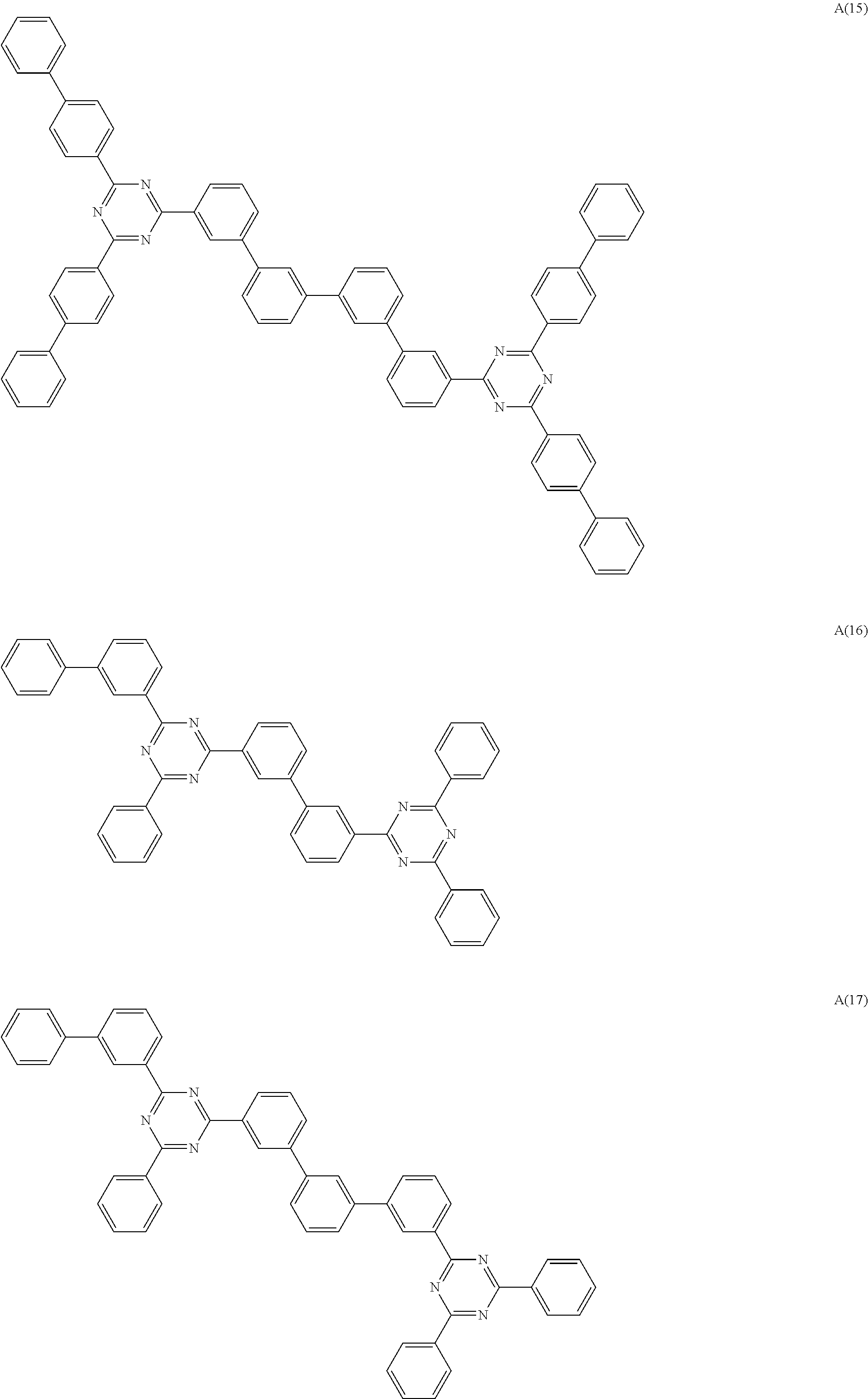 [SMILES: c1ccc(-c2ccc(-c3nc(-c4ccc(-c5ccccc5)cc4)nc(-c4cccc(-c5cccc(-c6cccc(-c7cccc(-c8nc(-c9ccc(-c%10ccccc%10)cc9)nc(-c9ccc(-c%10ccccc%10)cc9)n8)c7)c6)c5)c4)n3)cc2)cc1 ; c1ccc(-c2cccc(-c3nc(-c4ccccc4)nc(-c4cccc(-c5cccc(-c6nc(-c7ccccc7)nc(-c7ccccc7)n6)c5)c4)n3)c2)cc1 ; c1ccc(-c2cccc(-c3nc(-c4ccccc4)nc(-c4cccc(-c5cccc(-c6cccc(-c7nc(-c8ccccc8)nc(-c8ccccc8)n7)c6)c5)c4)n3)c2)cc1]

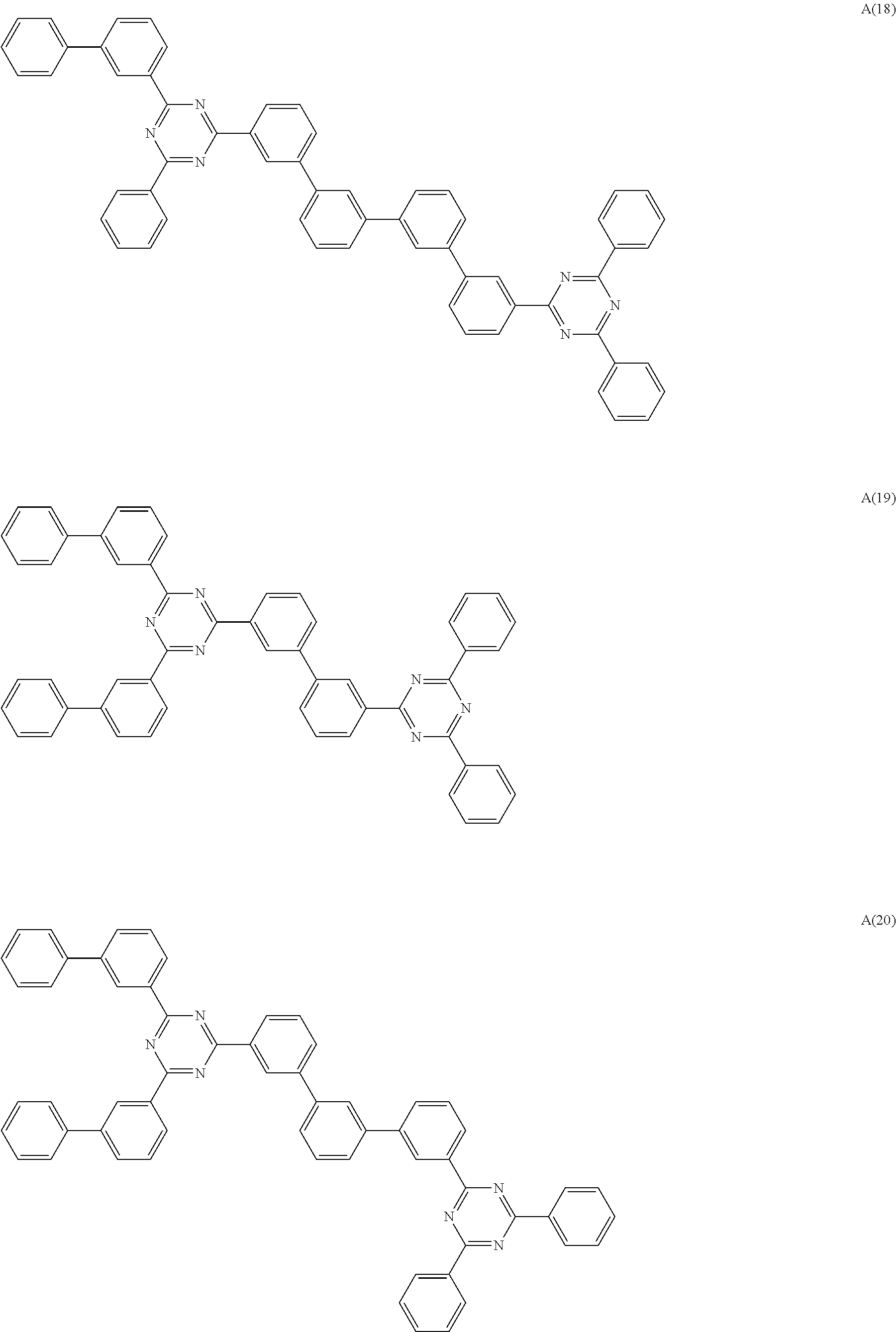 [SMILES: c1ccc(-c2cccc(-c3nc(-c4ccccc4)nc(-c4cccc(-c5cccc(-c6cccc(-c7cccc(-c8nc(-c9ccccc9)nc(-c9ccccc9)n8)c7)c6)c5)c4)n3)c2)cc1 ; c1ccc(-c2cccc(-c3nc(-c4cccc(-c5ccccc5)c4)nc(-c4cccc(-c5cccc(-c6nc(-c7ccccc7)nc(-c7ccccc7)n6)c5)c4)n3)c2)cc1 ; c1ccc(-c2cccc(-c3nc(-c4cccc(-c5ccccc5)c4)nc(-c4cccc(-c5cccc(-c6cccc(-c7nc(-c8ccccc8)nc(-c8ccccc8)n7)c6)c5)c4)n3)c2)cc1]

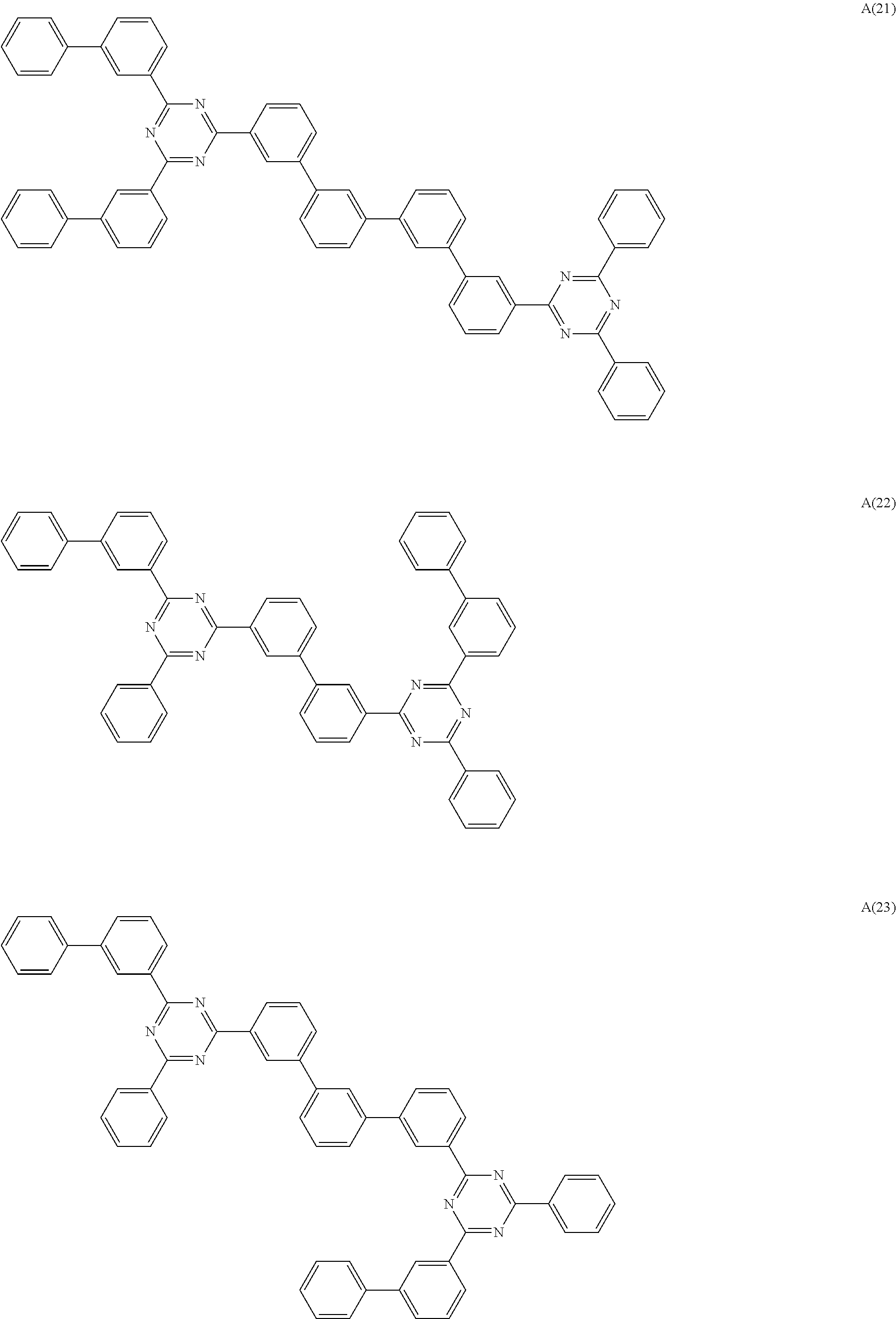 [SMILES: c1ccc(-c2cccc(-c3nc(-c4cccc(-c5ccccc5)c4)nc(-c4cccc(-c5cccc(-c6cccc(-c7cccc(-c8nc(-c9ccccc9)nc(-c9ccccc9)n8)c7)c6)c5)c4)n3)c2)cc1 ; c1ccc(-c2cccc(-c3nc(-c4ccccc4)nc(-c4cccc(-c5cccc(-c6nc(-c7ccccc7)nc(-c7cccc(-c8ccccc8)c7)n6)c5)c4)n3)c2)cc1 ; c1ccc(-c2cccc(-c3nc(-c4ccccc4)nc(-c4cccc(-c5cccc(-c6cccc(-c7nc(-c8ccccc8)nc(-c8cccc(-c9ccccc9)c8)n7)c6)c5)c4)n3)c2)cc1]

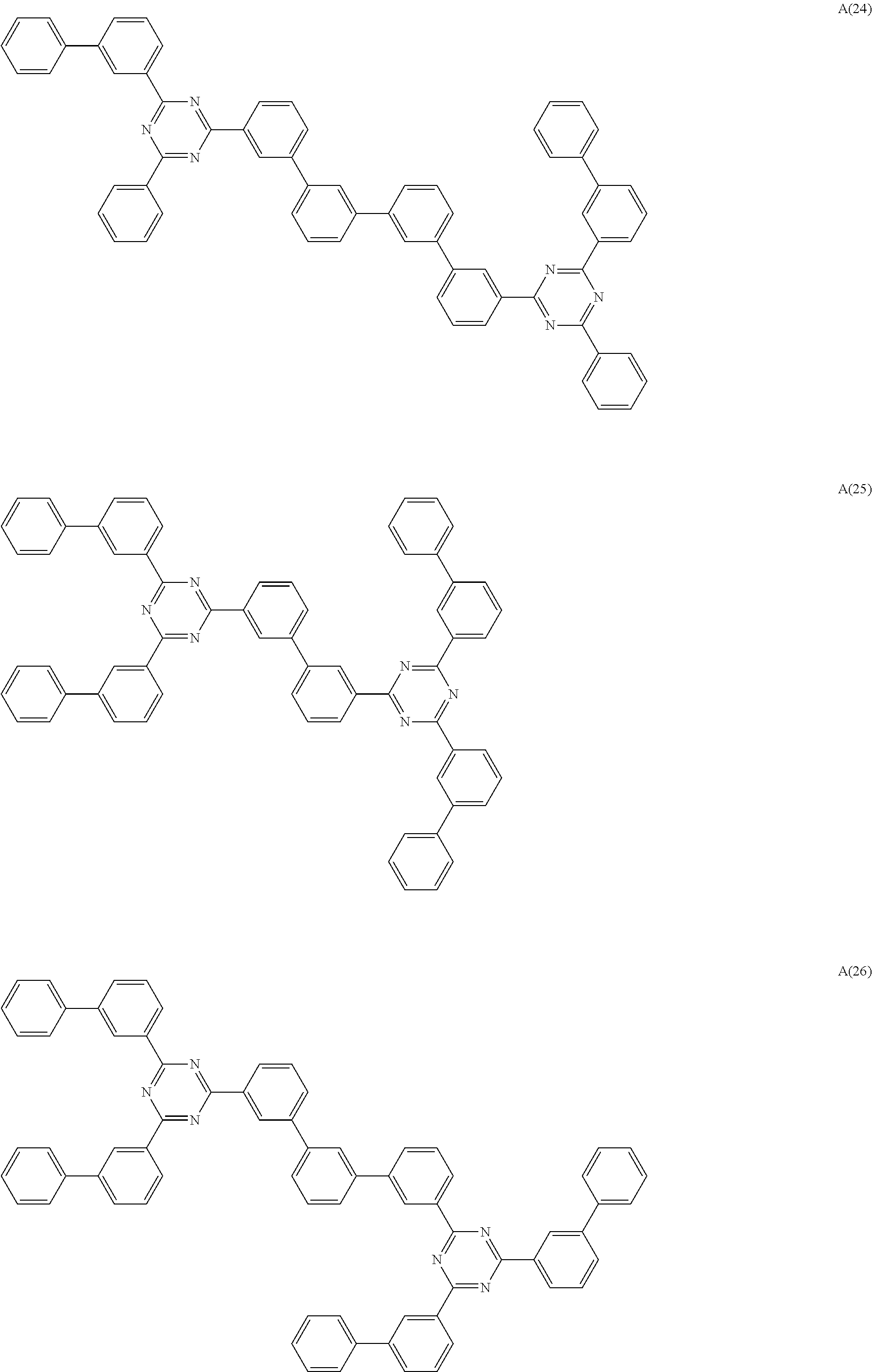 [SMILES: c1ccc(-c2cccc(-c3nc(-c4ccccc4)nc(-c4cccc(-c5cccc(-c6cccc(-c7cccc(-c8nc(-c9ccccc9)nc(-c9cccc(-c%10ccccc%10)c9)n8)c7)c6)c5)c4)n3)c2)cc1 ; c1ccc(-c2cccc(-c3nc(-c4cccc(-c5ccccc5)c4)nc(-c4cccc(-c5cccc(-c6nc(-c7cccc(-c8ccccc8)c7)nc(-c7cccc(-c8ccccc8)c7)n6)c5)c4)n3)c2)cc1 ; c1ccc(-c2cccc(-c3nc(-c4cccc(-c5ccccc5)c4)nc(-c4cccc(-c5cccc(-c6cccc(-c7nc(-c8cccc(-c9ccccc9)c8)nc(-c8cccc(-c9ccccc9)c8)n7)c6)c5)c4)n3)c2)cc1]

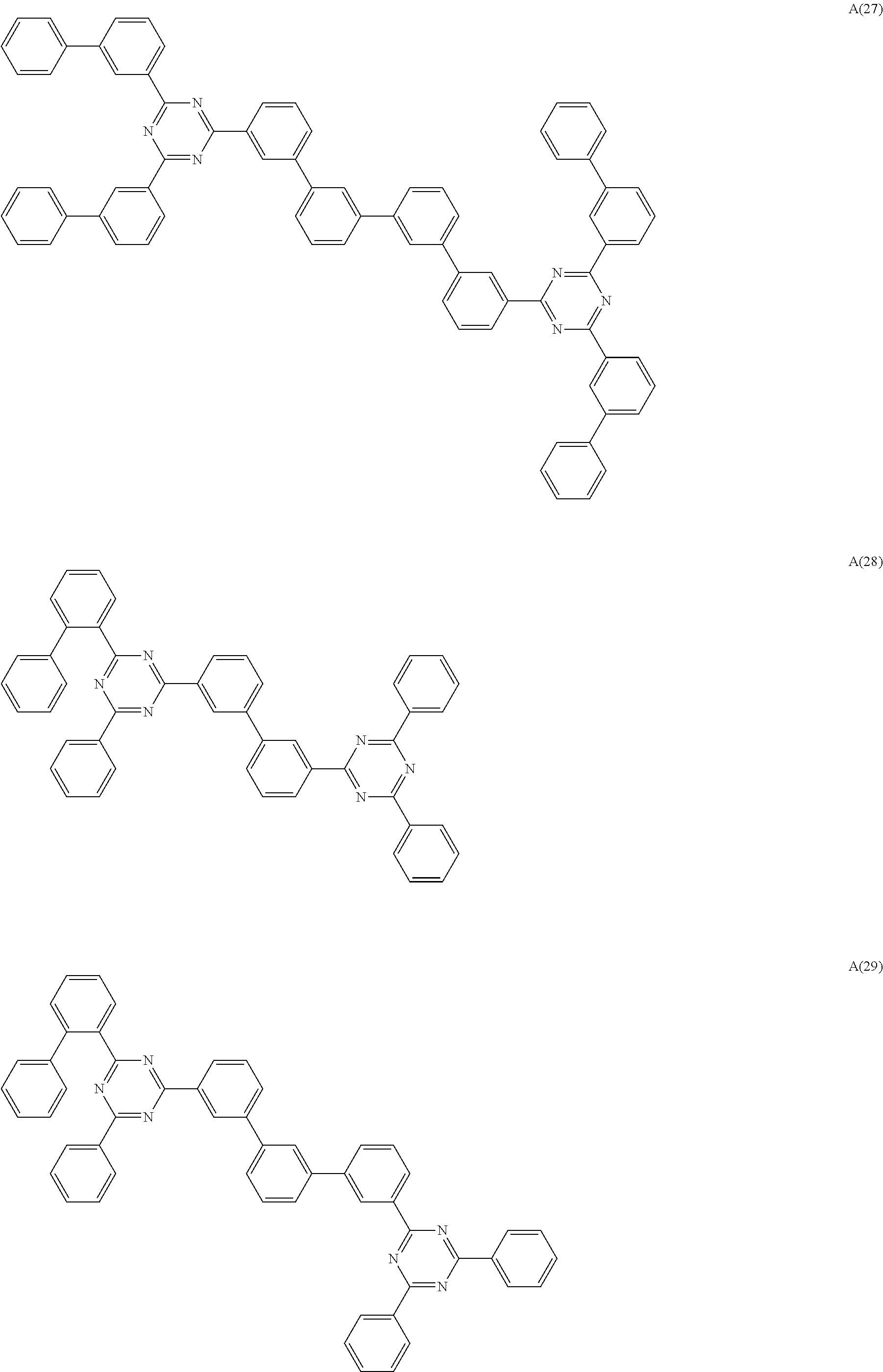 [SMILES: c1ccc(-c2cccc(-c3nc(-c4cccc(-c5ccccc5)c4)nc(-c4cccc(-c5cccc(-c6cccc(-c7cccc(-c8nc(-c9cccc(-c%10ccccc%10)c9)nc(-c9cccc(-c%10ccccc%10)c9)n8)c7)c6)c5)c4)n3)c2)cc1 ; c1ccc(-c2nc(-c3ccccc3)nc(-c3cccc(-c4cccc(-c5nc(-c6ccccc6)nc(-c6ccccc6-c6ccccc6)n5)c4)c3)n2)cc1 ; c1ccc(-c2nc(-c3ccccc3)nc(-c3cccc(-c4cccc(-c5cccc(-c6nc(-c7ccccc7)nc(-c7ccccc7-c7ccccc7)n6)c5)c4)c3)n2)cc1]

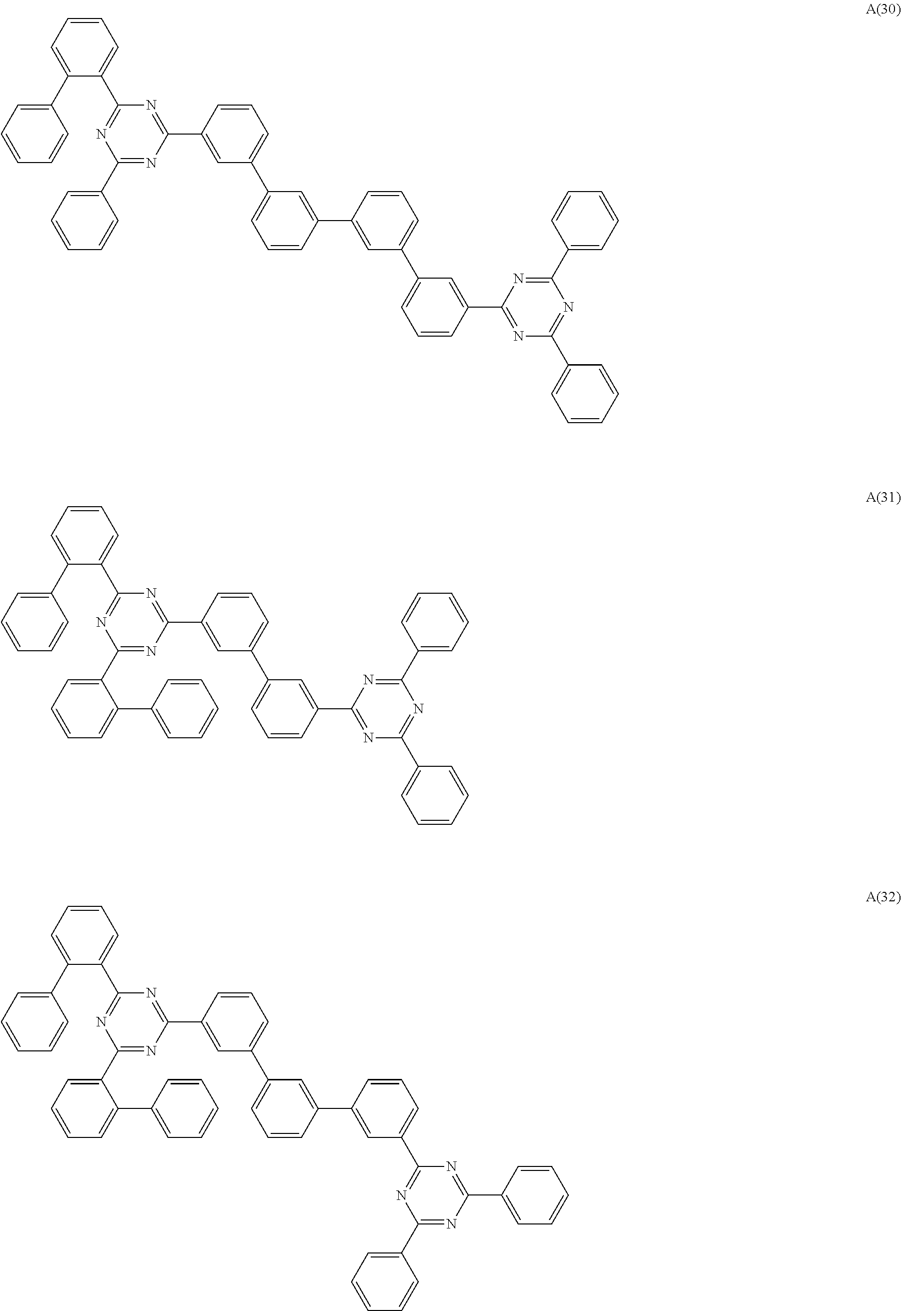 [SMILES: c1ccc(-c2nc(-c3ccccc3)nc(-c3cccc(-c4cccc(-c5cccc(-c6cccc(-c7nc(-c8ccccc8)nc(-c8ccccc8-c8ccccc8)n7)c6)c5)c4)c3)n2)cc1 ; c1ccc(-c2nc(-c3ccccc3)nc(-c3cccc(-c4cccc(-c5nc(-c6ccccc6-c6ccccc6)nc(-c6ccccc6-c6ccccc6)n5)c4)c3)n2)cc1 ; c1ccc(-c2nc(-c3ccccc3)nc(-c3cccc(-c4cccc(-c5cccc(-c6nc(-c7ccccc7-c7ccccc7)nc(-c7ccccc7-c7ccccc7)n6)c5)c4)c3)n2)cc1]

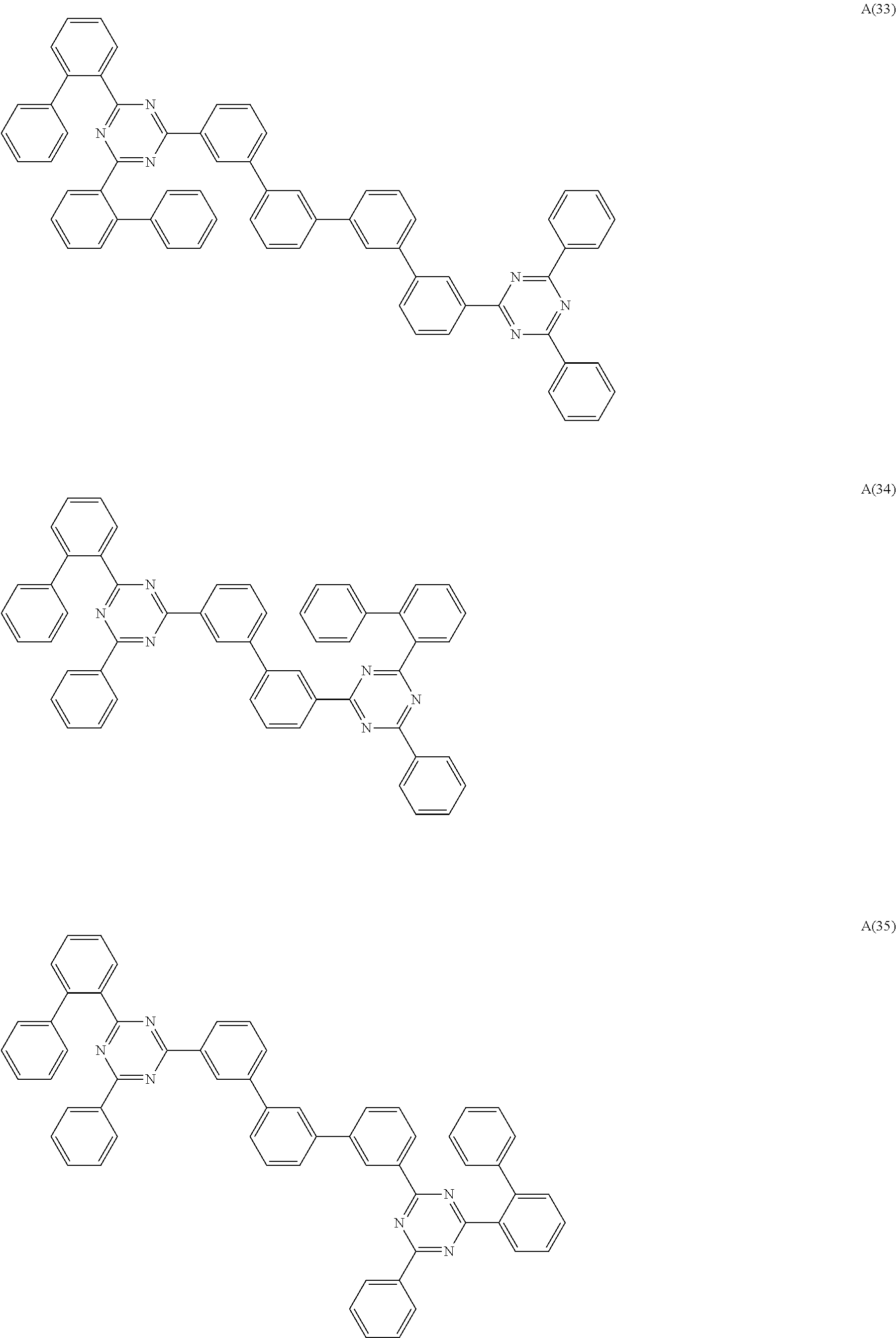 [SMILES: c1ccc(-c2nc(-c3ccccc3)nc(-c3cccc(-c4cccc(-c5cccc(-c6cccc(-c7nc(-c8ccccc8-c8ccccc8)nc(-c8ccccc8-c8ccccc8)n7)c6)c5)c4)c3)n2)cc1 ; c1ccc(-c2nc(-c3cccc(-c4cccc(-c5nc(-c6ccccc6)nc(-c6ccccc6-c6ccccc6)n5)c4)c3)nc(-c3ccccc3-c3ccccc3)n2)cc1 ; c1ccc(-c2nc(-c3cccc(-c4cccc(-c5cccc(-c6nc(-c7ccccc7)nc(-c7ccccc7-c7ccccc7)n6)c5)c4)c3)nc(-c3ccccc3-c3ccccc3)n2)cc1]

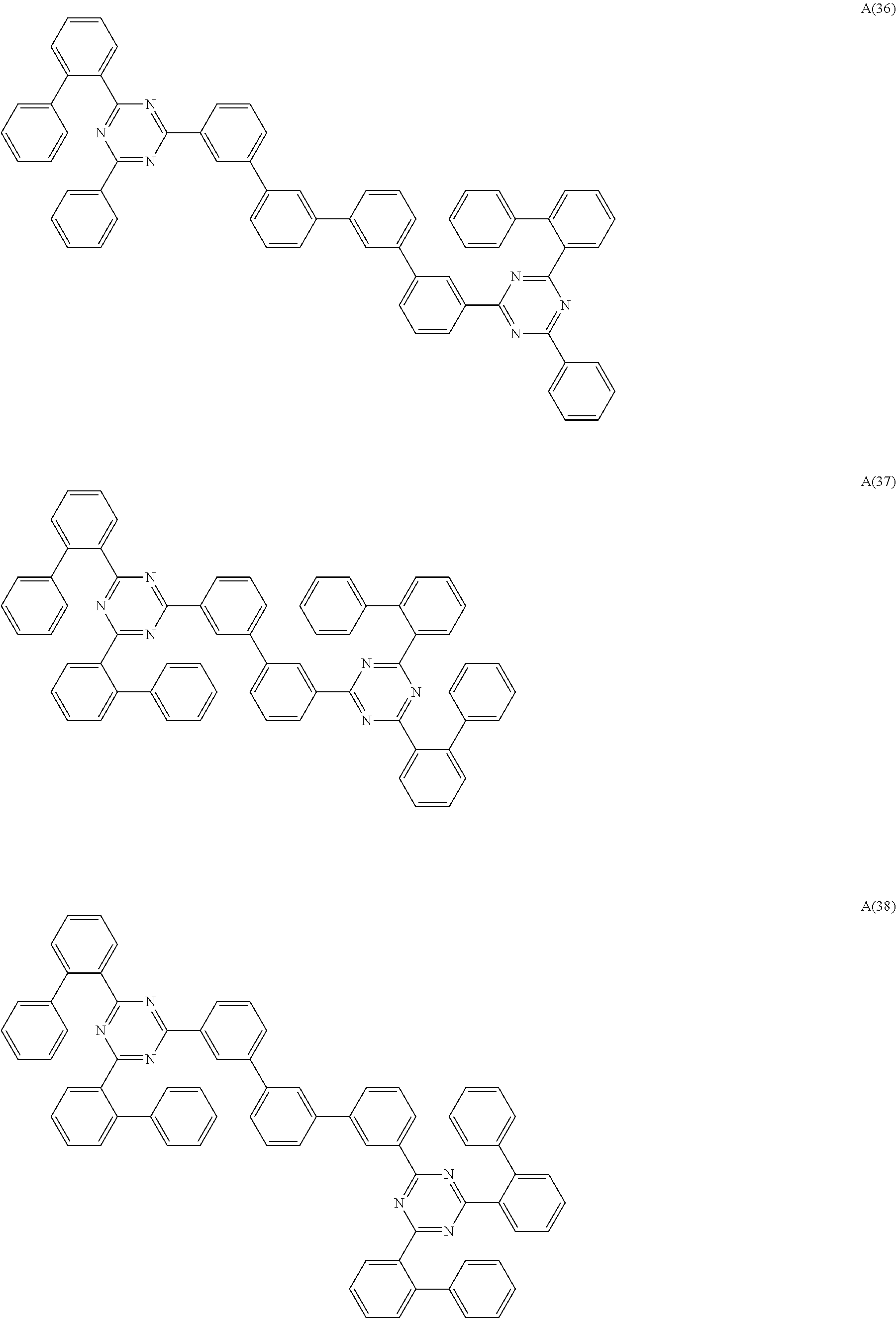 [SMILES: c1ccc(-c2nc(-c3cccc(-c4cccc(-c5cccc(-c6cccc(-c7nc(-c8ccccc8)nc(-c8ccccc8-c8ccccc8)n7)c6)c5)c4)c3)nc(-c3ccccc3-c3ccccc3)n2)cc1 ; c1ccc(-c2ccccc2-c2nc(-c3cccc(-c4cccc(-c5nc(-c6ccccc6-c6ccccc6)nc(-c6ccccc6-c6ccccc6)n5)c4)c3)nc(-c3ccccc3-c3ccccc3)n2)cc1 ; c1ccc(-c2ccccc2-c2nc(-c3cccc(-c4cccc(-c5cccc(-c6nc(-c7ccccc7-c7ccccc7)nc(-c7ccccc7-c7ccccc7)n6)c5)c4)c3)nc(-c3ccccc3-c3ccccc3)n2)cc1]

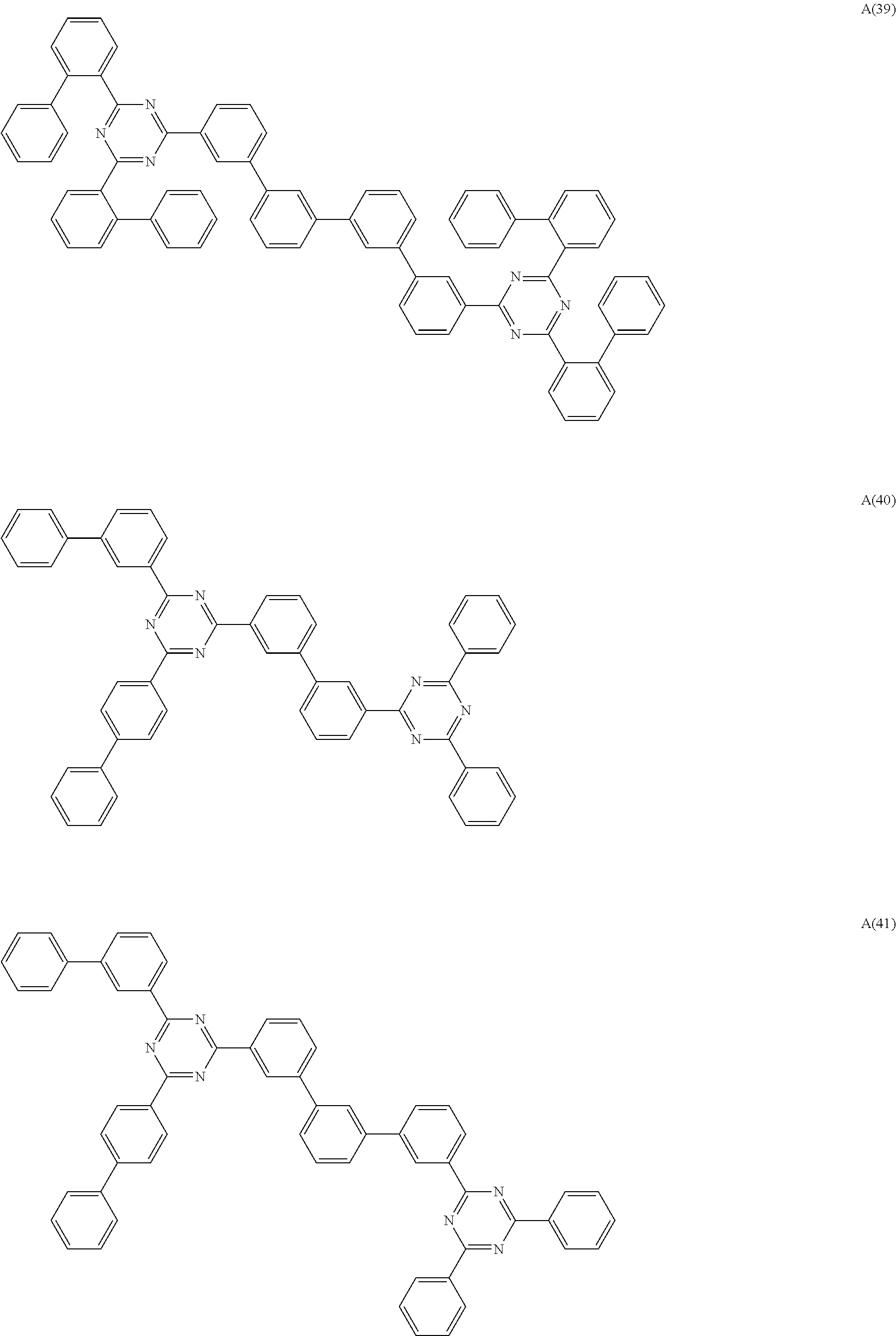 [SMILES: c1ccc(-c2ccccc2-c2nc(-c3cccc(-c4cccc(-c5cccc(-c6cccc(-c7nc(-c8ccccc8-c8ccccc8)nc(-c8ccccc8-c8ccccc8)n7)c6)c5)c4)c3)nc(-c3ccccc3-c3ccccc3)n2)cc1 ; c1ccc(-c2ccc(-c3nc(-c4cccc(-c5ccccc5)c4)nc(-c4cccc(-c5cccc(-c6nc(-c7ccccc7)nc(-c7ccccc7)n6)c5)c4)n3)cc2)cc1 ; c1ccc(-c2ccc(-c3nc(-c4cccc(-c5ccccc5)c4)nc(-c4cccc(-c5cccc(-c6cccc(-c7nc(-c8ccccc8)nc(-c8ccccc8)n7)c6)c5)c4)n3)cc2)cc1]

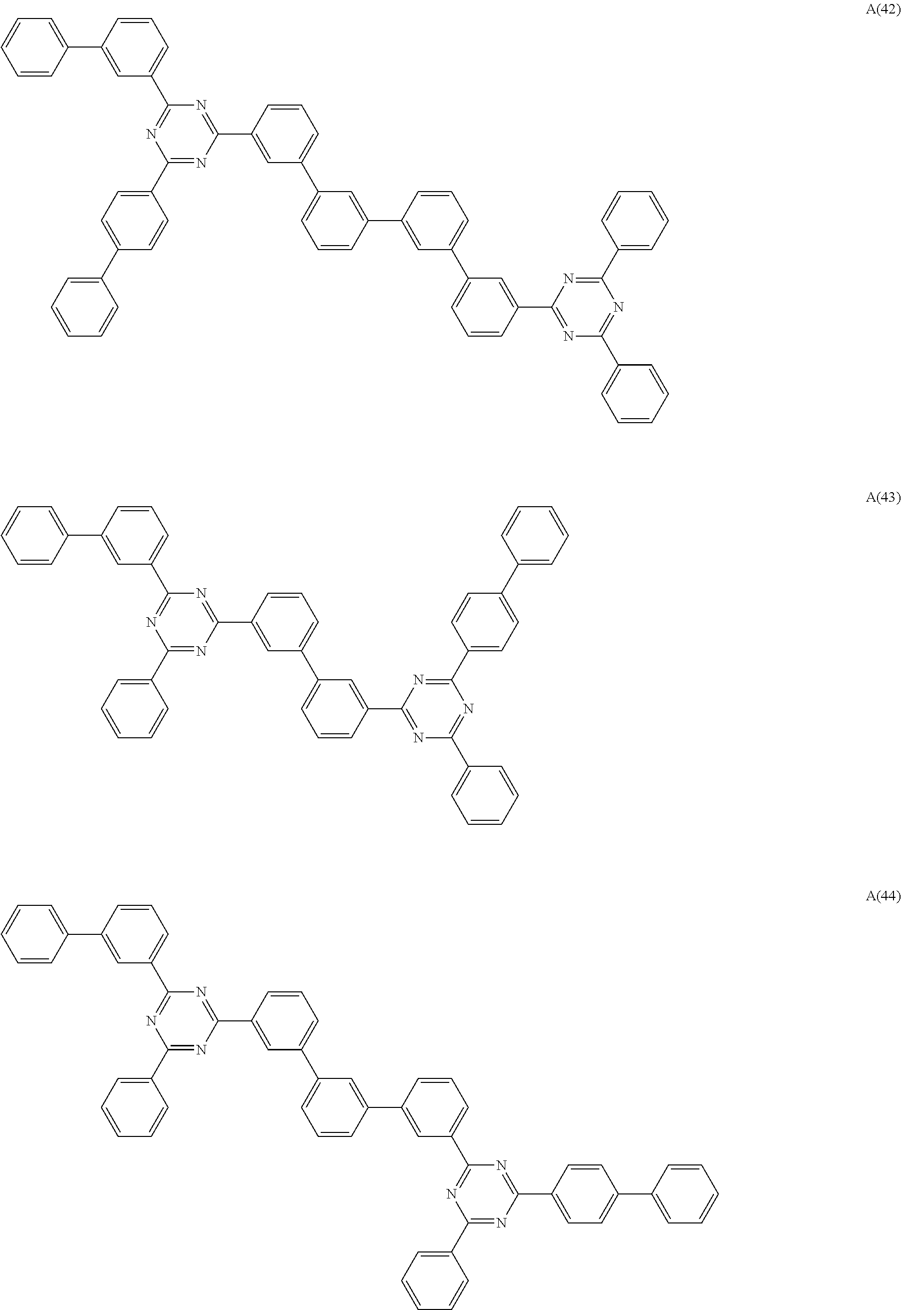 [SMILES: c1ccc(-c2ccc(-c3nc(-c4cccc(-c5ccccc5)c4)nc(-c4cccc(-c5cccc(-c6cccc(-c7cccc(-c8nc(-c9ccccc9)nc(-c9ccccc9)n8)c7)c6)c5)c4)n3)cc2)cc1 ; c1ccc(-c2ccc(-c3nc(-c4ccccc4)nc(-c4cccc(-c5cccc(-c6nc(-c7ccccc7)nc(-c7cccc(-c8ccccc8)c7)n6)c5)c4)n3)cc2)cc1 ; c1ccc(-c2ccc(-c3nc(-c4ccccc4)nc(-c4cccc(-c5cccc(-c6cccc(-c7nc(-c8ccccc8)nc(-c8cccc(-c9ccccc9)c8)n7)c6)c5)c4)n3)cc2)cc1]

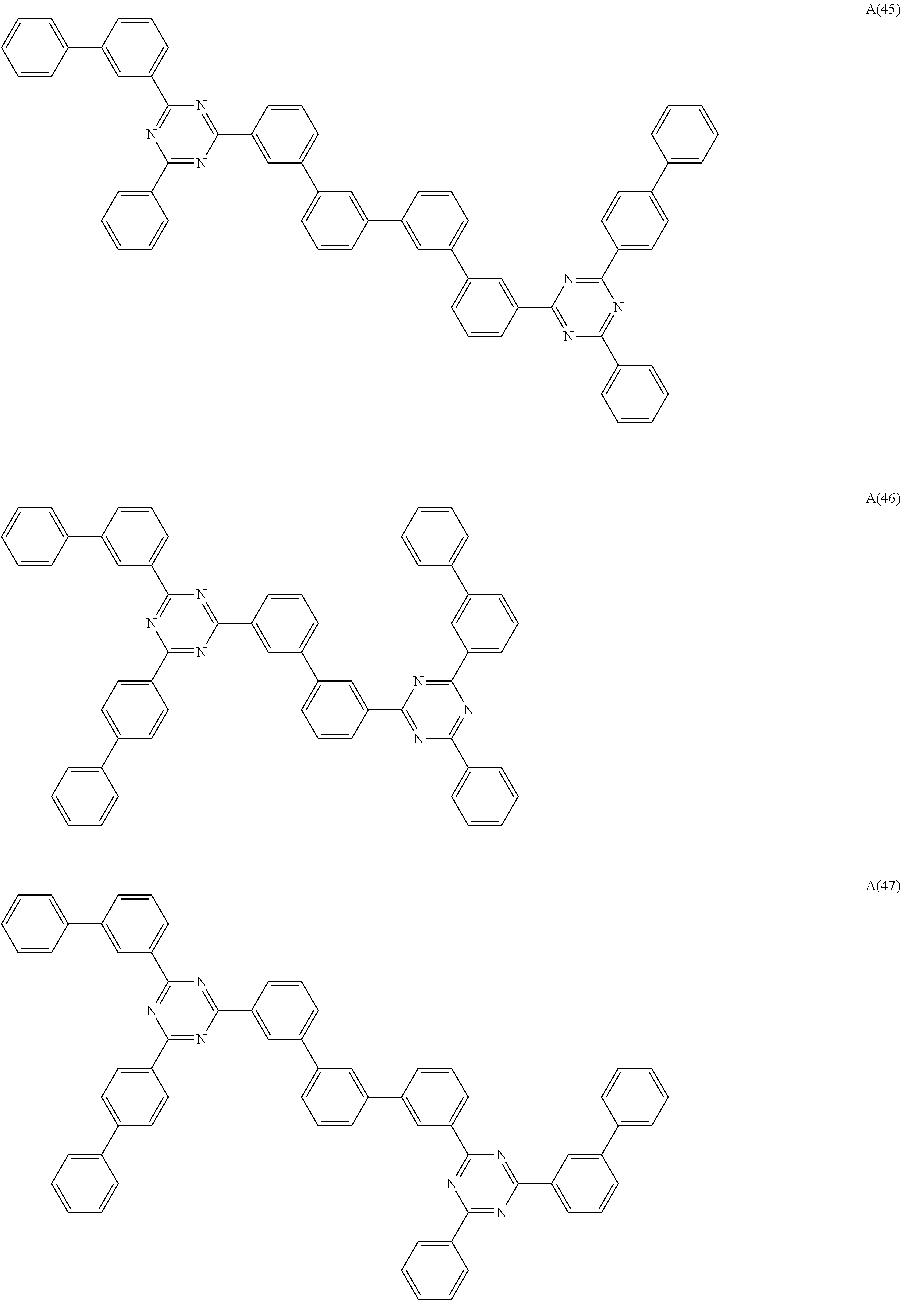 [SMILES: c1ccc(-c2ccc(-c3nc(-c4ccccc4)nc(-c4cccc(-c5cccc(-c6cccc(-c7cccc(-c8nc(-c9ccccc9)nc(-c9cccc(-c%10ccccc%10)c9)n8)c7)c6)c5)c4)n3)cc2)cc1 ; c1ccc(-c2ccc(-c3nc(-c4cccc(-c5ccccc5)c4)nc(-c4cccc(-c5cccc(-c6nc(-c7ccccc7)nc(-c7cccc(-c8ccccc8)c7)n6)c5)c4)n3)cc2)cc1 ; c1ccc(-c2ccc(-c3nc(-c4cccc(-c5ccccc5)c4)nc(-c4cccc(-c5cccc(-c6cccc(-c7nc(-c8ccccc8)nc(-c8cccc(-c9ccccc9)c8)n7)c6)c5)c4)n3)cc2)cc1]

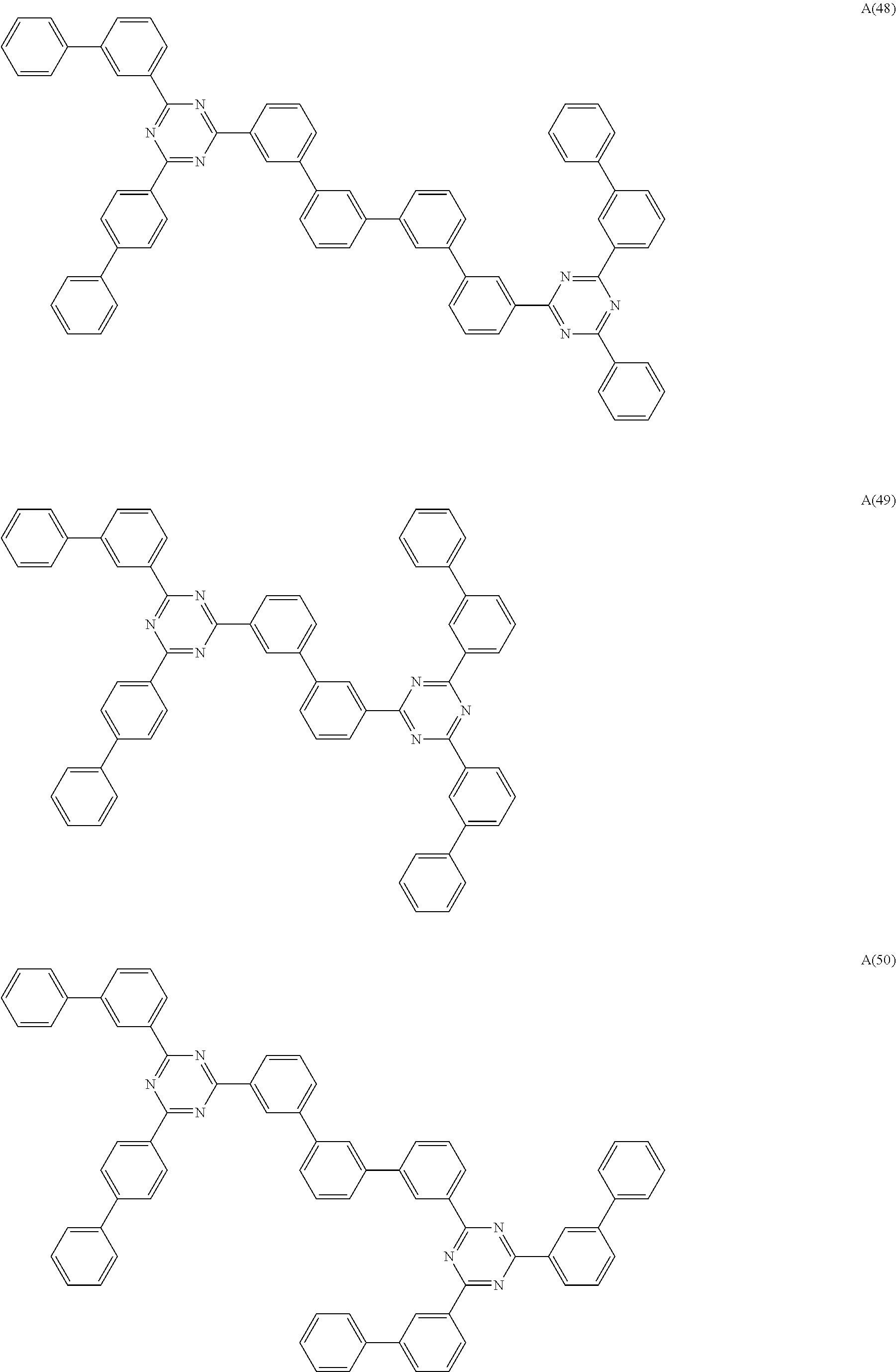 [SMILES: c1ccc(-c2ccc(-c3nc(-c4cccc(-c5ccccc5)c4)nc(-c4cccc(-c5cccc(-c6cccc(-c7cccc(-c8nc(-c9ccccc9)nc(-c9cccc(-c%10ccccc%10)c9)n8)c7)c6)c5)c4)n3)cc2)cc1 ; c1ccc(-c2ccc(-c3nc(-c4cccc(-c5ccccc5)c4)nc(-c4cccc(-c5cccc(-c6nc(-c7cccc(-c8ccccc8)c7)nc(-c7cccc(-c8ccccc8)c7)n6)c5)c4)n3)cc2)cc1 ; c1ccc(-c2ccc(-c3nc(-c4cccc(-c5ccccc5)c4)nc(-c4cccc(-c5cccc(-c6cccc(-c7nc(-c8cccc(-c9ccccc9)c8)nc(-c8cccc(-c9ccccc9)c8)n7)c6)c5)c4)n3)cc2)cc1]

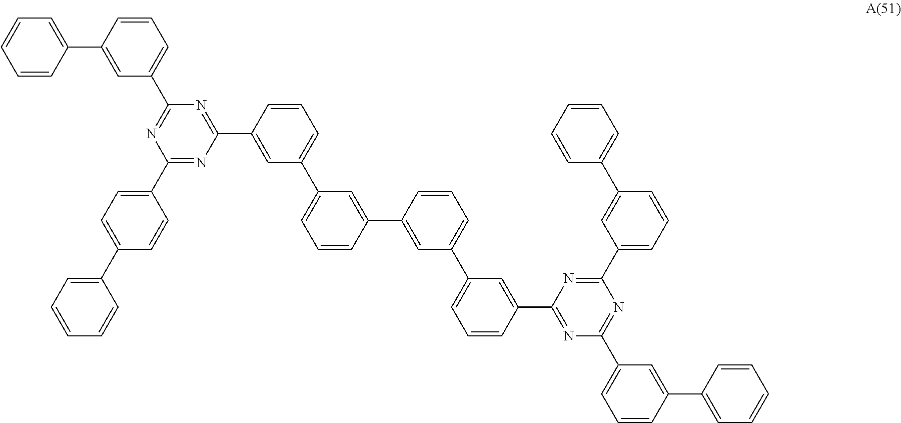 [SMILES: c1ccc(-c2ccc(-c3nc(-c4cccc(-c5ccccc5)c4)nc(-c4cccc(-c5cccc(-c6cccc(-c7cccc(-c8nc(-c9cccc(-c%10ccccc%10)c9)nc(-c9cccc(-c%10ccccc%10)c9)n8)c7)c6)c5)c4)n3)cc2)cc1]

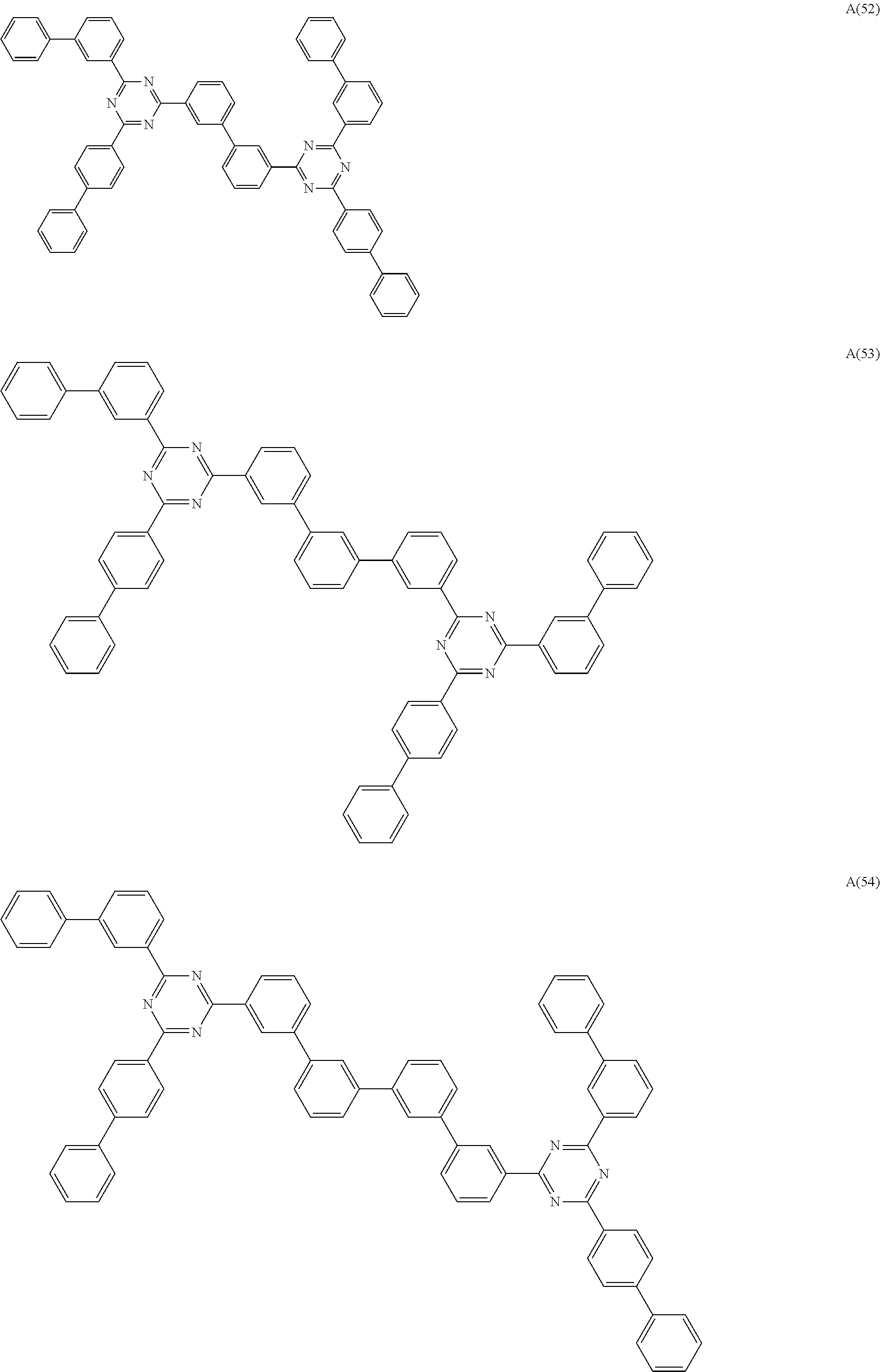 [SMILES: c1ccc(-c2ccc(-c3nc(-c4cccc(-c5ccccc5)c4)nc(-c4cccc(-c5cccc(-c6nc(-c7ccc(-c8ccccc8)cc7)nc(-c7cccc(-c8ccccc8)c7)n6)c5)c4)n3)cc2)cc1 ; c1ccc(-c2ccc(-c3nc(-c4cccc(-c5ccccc5)c4)nc(-c4cccc(-c5cccc(-c6cccc(-c7nc(-c8ccc(-c9ccccc9)cc8)nc(-c8cccc(-c9ccccc9)c8)n7)c6)c5)c4)n3)cc2)cc1 ; c1ccc(-c2ccc(-c3nc(-c4cccc(-c5ccccc5)c4)nc(-c4cccc(-c5cccc(-c6cccc(-c7cccc(-c8nc(-c9ccc(-c%10ccccc%10)cc9)nc(-c9cccc(-c%10ccccc%10)c9)n8)c7)c6)c5)c4)n3)cc2)cc1]

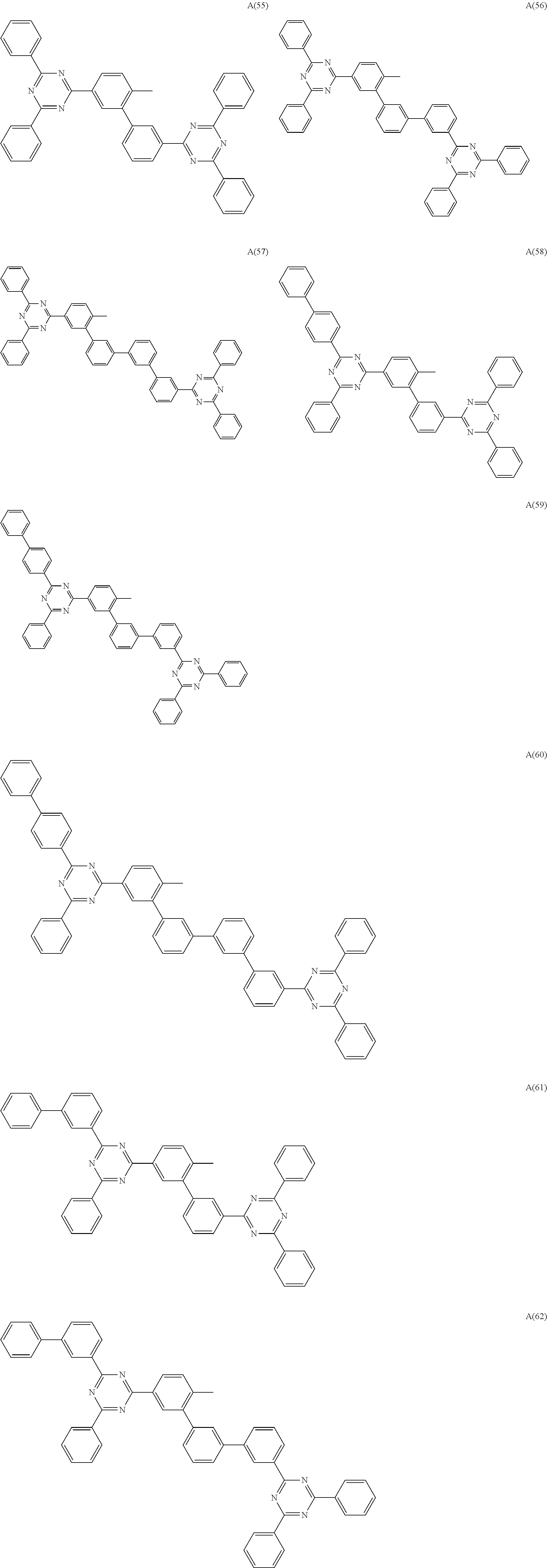

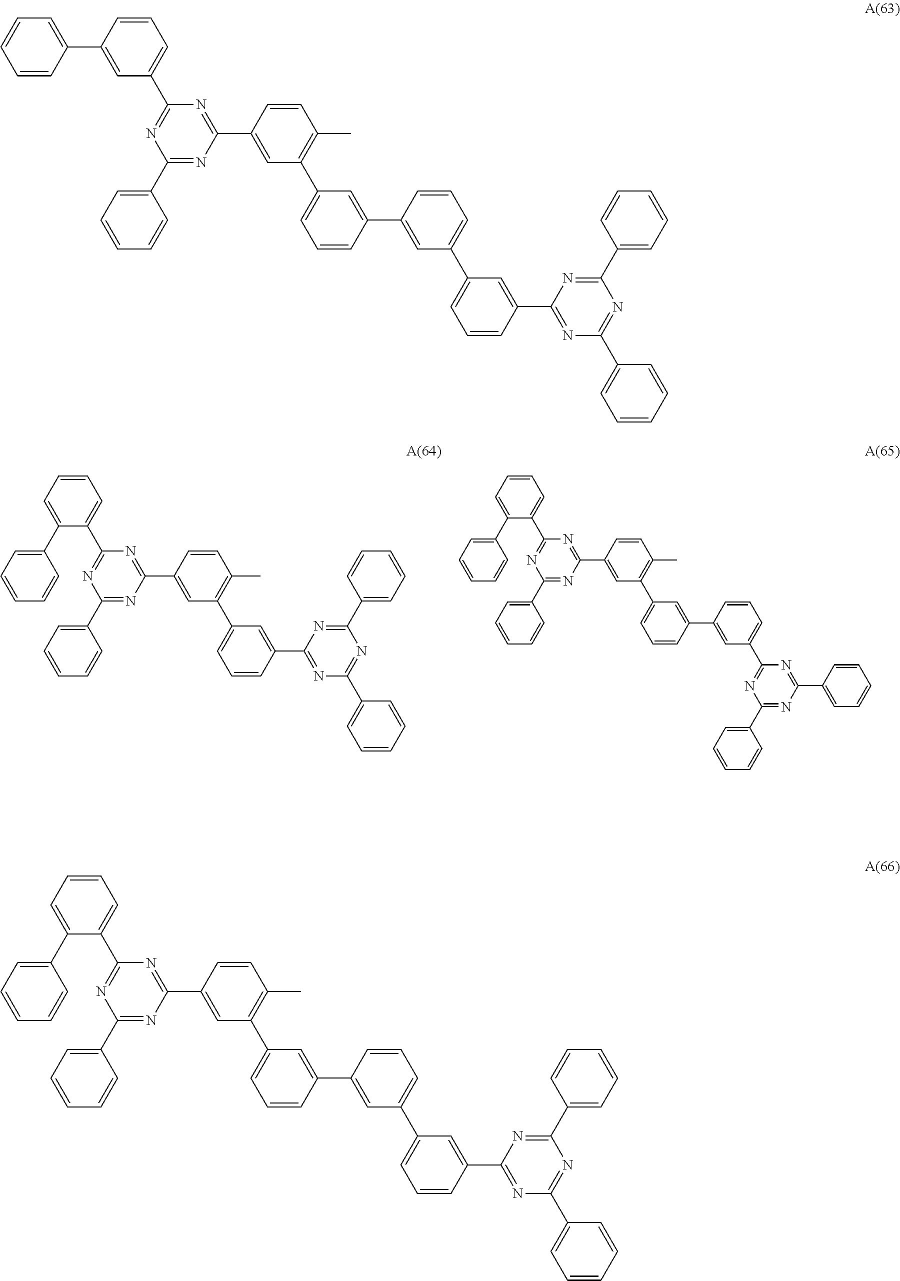 [SMILES: Cc1ccc(-c2nc(-c3ccccc3)nc(-c3cccc(-c4ccccc4)c3)n2)cc1-c1cccc(-c2cccc(-c3cccc(-c4nc(-c5ccccc5)nc(-c5ccccc5)n4)c3)c2)c1 ; Cc1ccc(-c2nc(-c3ccccc3)nc(-c3ccccc3-c3ccccc3)n2)cc1-c1cccc(-c2nc(-c3ccccc3)nc(-c3ccccc3)n2)c1 ; Cc1ccc(-c2nc(-c3ccccc3)nc(-c3ccccc3-c3ccccc3)n2)cc1-c1cccc(-c2cccc(-c3nc(-c4ccccc4)nc(-c4ccccc4)n3)c2)c1 ; Cc1ccc(-c2nc(-c3ccccc3)nc(-c3ccccc3-c3ccccc3)n2)cc1-c1cccc(-c2cccc(-c3cccc(-c4nc(-c5ccccc5)nc(-c5ccccc5)n4)c3)c2)c1]

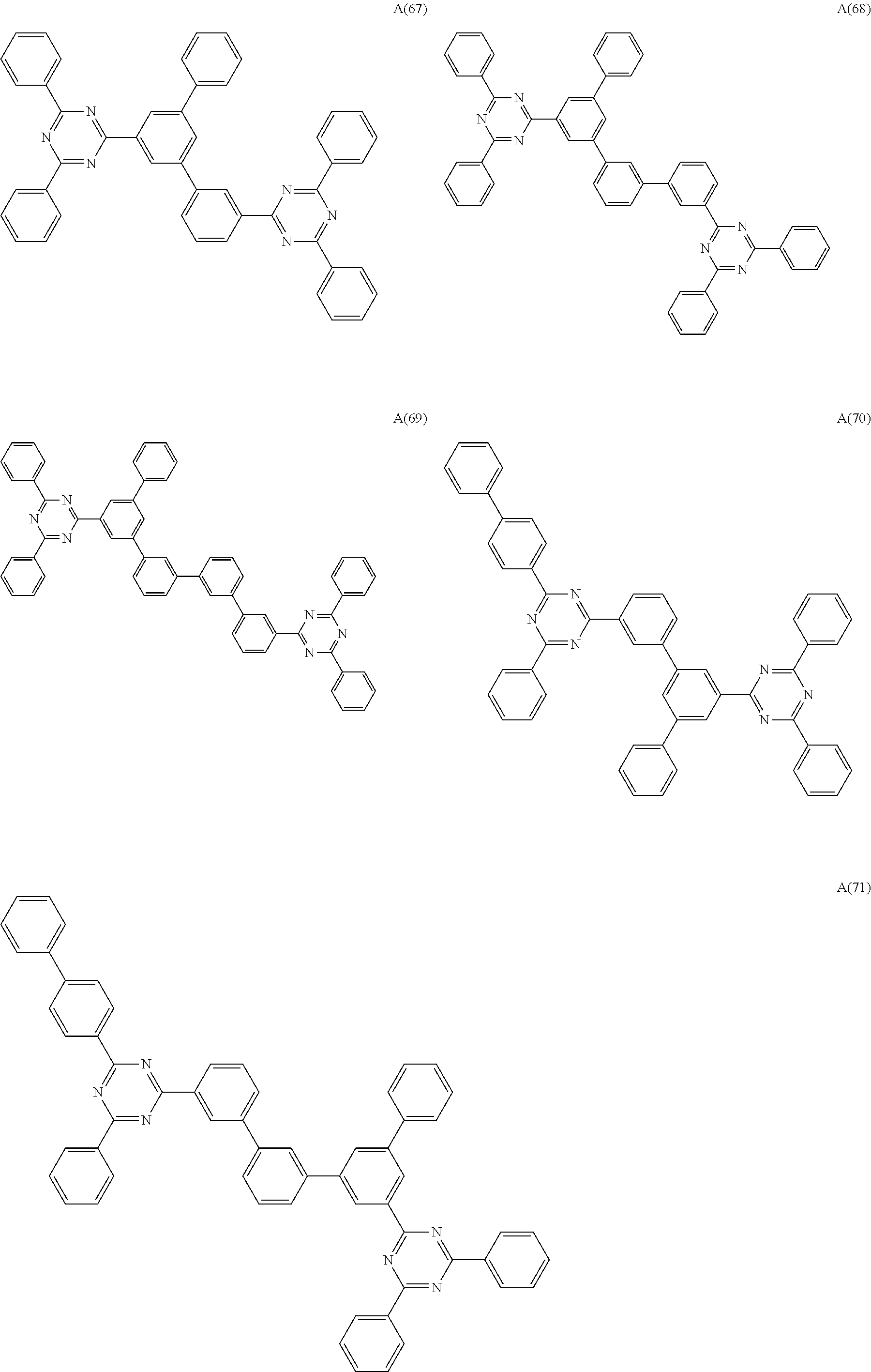

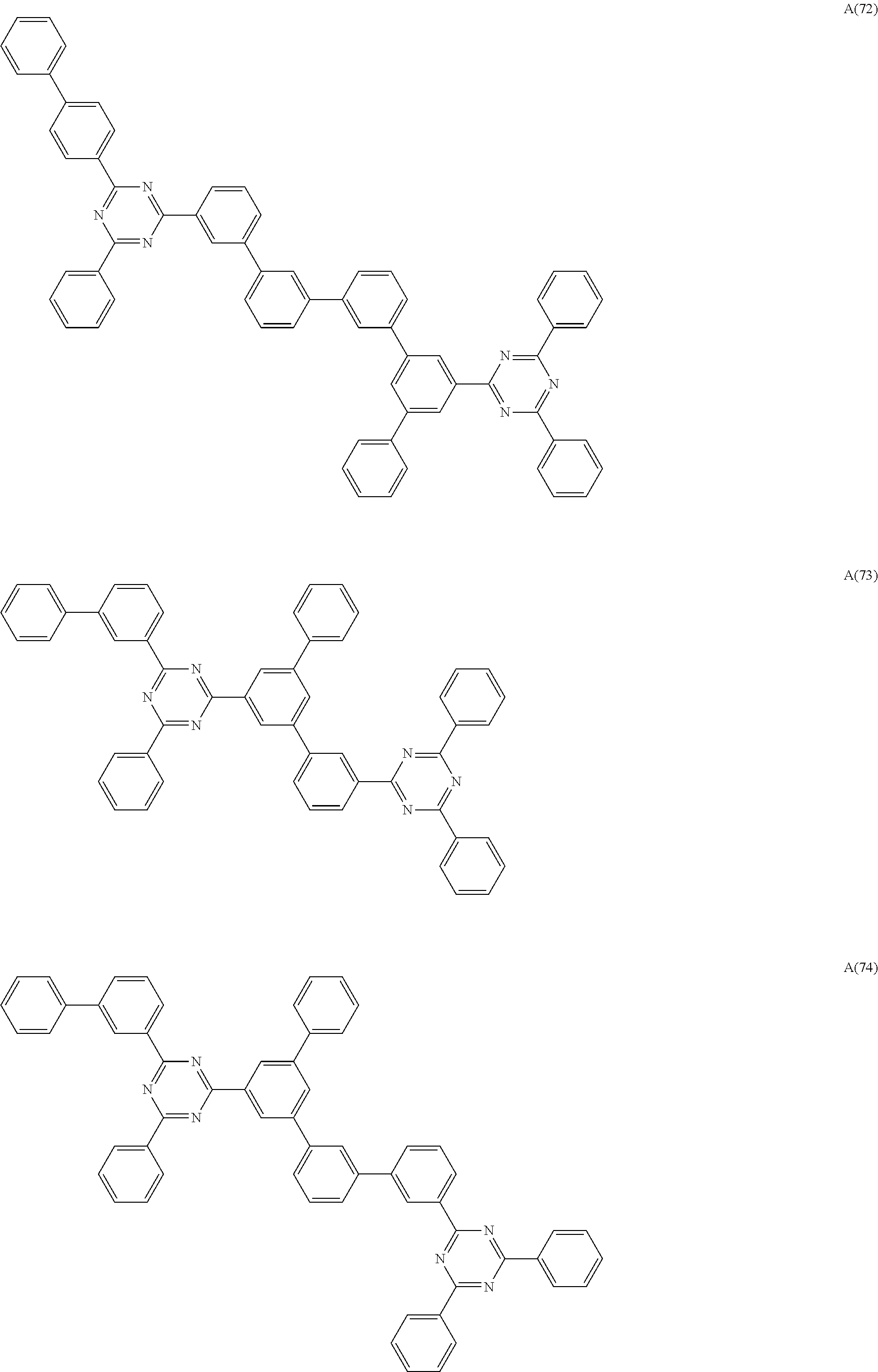 [SMILES: c1ccc(-c2ccc(-c3nc(-c4ccccc4)nc(-c4cccc(-c5cccc(-c6cccc(-c7cc(-c8ccccc8)cc(-c8nc(-c9ccccc9)nc(-c9ccccc9)n8)c7)c6)c5)c4)n3)cc2)cc1 ; c1ccc(-c2cccc(-c3nc(-c4ccccc4)nc(-c4cc(-c5ccccc5)cc(-c5cccc(-c6nc(-c7ccccc7)nc(-c7ccccc7)n6)c5)c4)n3)c2)cc1 ; c1ccc(-c2cccc(-c3nc(-c4ccccc4)nc(-c4cc(-c5ccccc5)cc(-c5cccc(-c6cccc(-c7nc(-c8ccccc8)nc(-c8ccccc8)n7)c6)c5)c4)n3)c2)cc1]

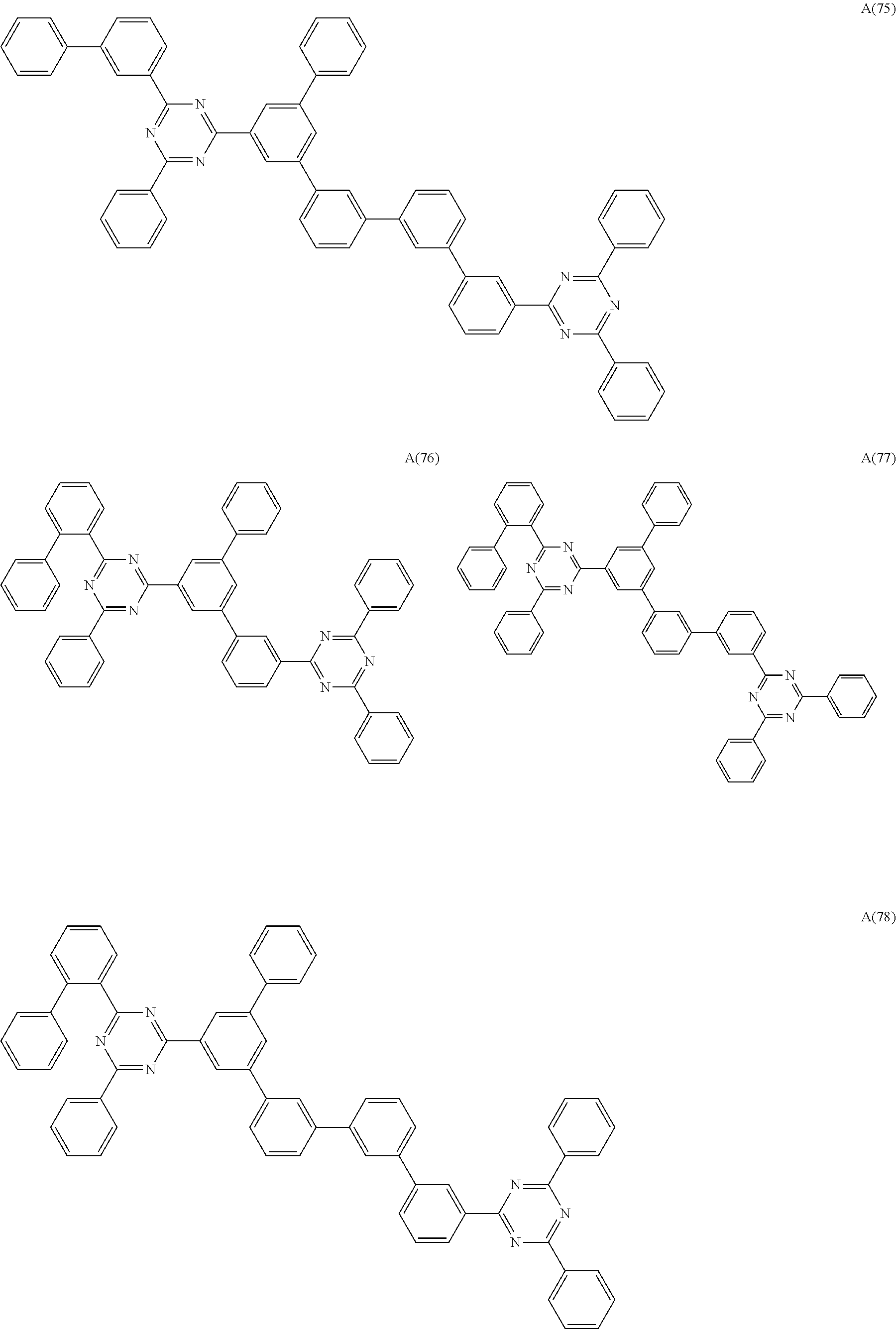 [SMILES: c1ccc(-c2cccc(-c3nc(-c4ccccc4)nc(-c4cc(-c5ccccc5)cc(-c5cccc(-c6cccc(-c7cccc(-c8nc(-c9ccccc9)nc(-c9ccccc9)n8)c7)c6)c5)c4)n3)c2)cc1 ; c1ccc(-c2cc(-c3cccc(-c4nc(-c5ccccc5)nc(-c5ccccc5)n4)c3)cc(-c3nc(-c4ccccc4)nc(-c4ccccc4-c4ccccc4)n3)c2)cc1 ; c1ccc(-c2cc(-c3cccc(-c4cccc(-c5nc(-c6ccccc6)nc(-c6ccccc6)n5)c4)c3)cc(-c3nc(-c4ccccc4)nc(-c4ccccc4-c4ccccc4)n3)c2)cc1 ; c1ccc(-c2cc(-c3cccc(-c4cccc(-c5cccc(-c6nc(-c7ccccc7)nc(-c7ccccc7)n6)c5)c4)c3)cc(-c3nc(-c4ccccc4)nc(-c4ccccc4-c4ccccc4)n3)c2)cc1]

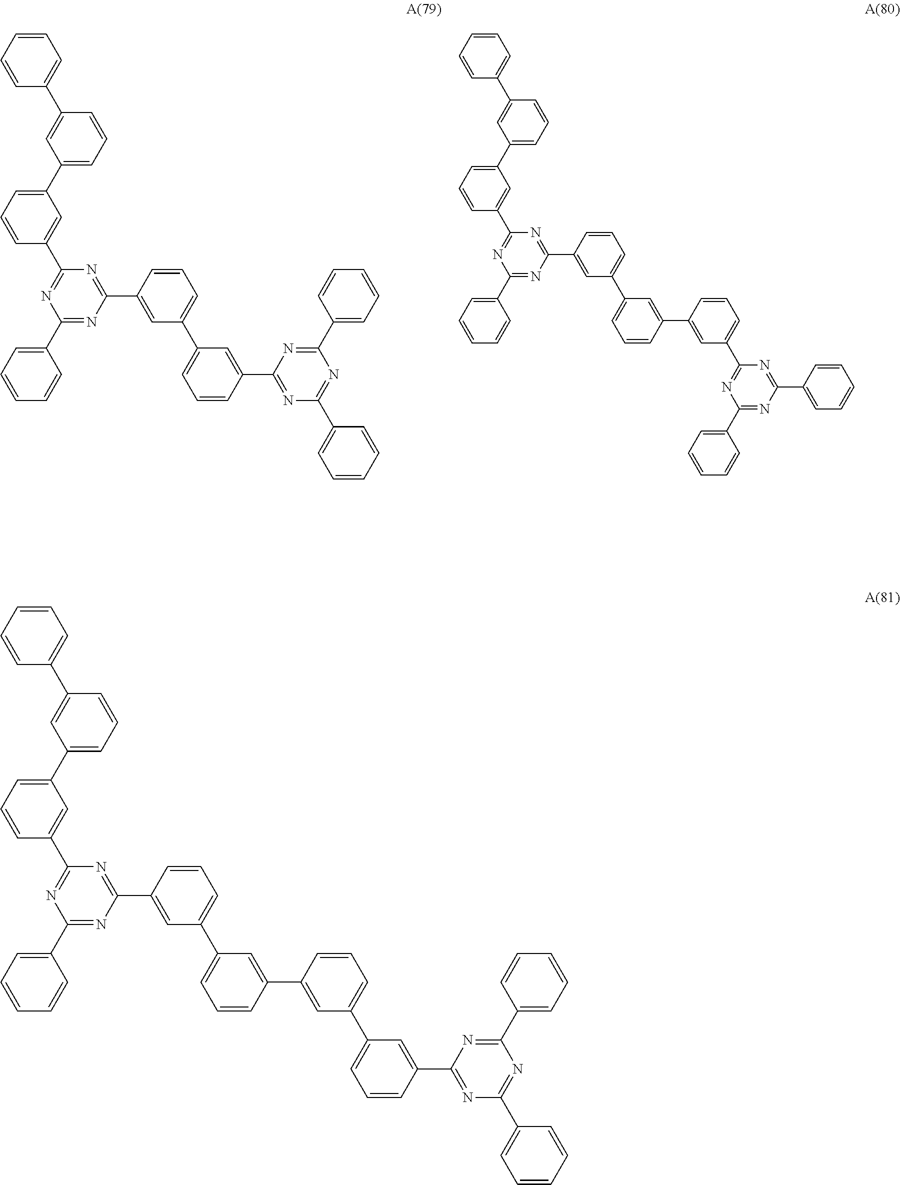 [SMILES: c1ccc(-c2cccc(-c3cccc(-c4nc(-c5ccccc5)nc(-c5cccc(-c6cccc(-c7nc(-c8ccccc8)nc(-c8ccccc8)n7)c6)c5)n4)c3)c2)cc1 ; c1ccc(-c2cccc(-c3cccc(-c4nc(-c5ccccc5)nc(-c5cccc(-c6cccc(-c7cccc(-c8nc(-c9ccccc9)nc(-c9ccccc9)n8)c7)c6)c5)n4)c3)c2)cc1 ; c1ccc(-c2cccc(-c3cccc(-c4nc(-c5ccccc5)nc(-c5cccc(-c6cccc(-c7cccc(-c8cccc(-c9nc(-c%10ccccc%10)nc(-c%10ccccc%10)n9)c8)c7)c6)c5)n4)c3)c2)cc1]

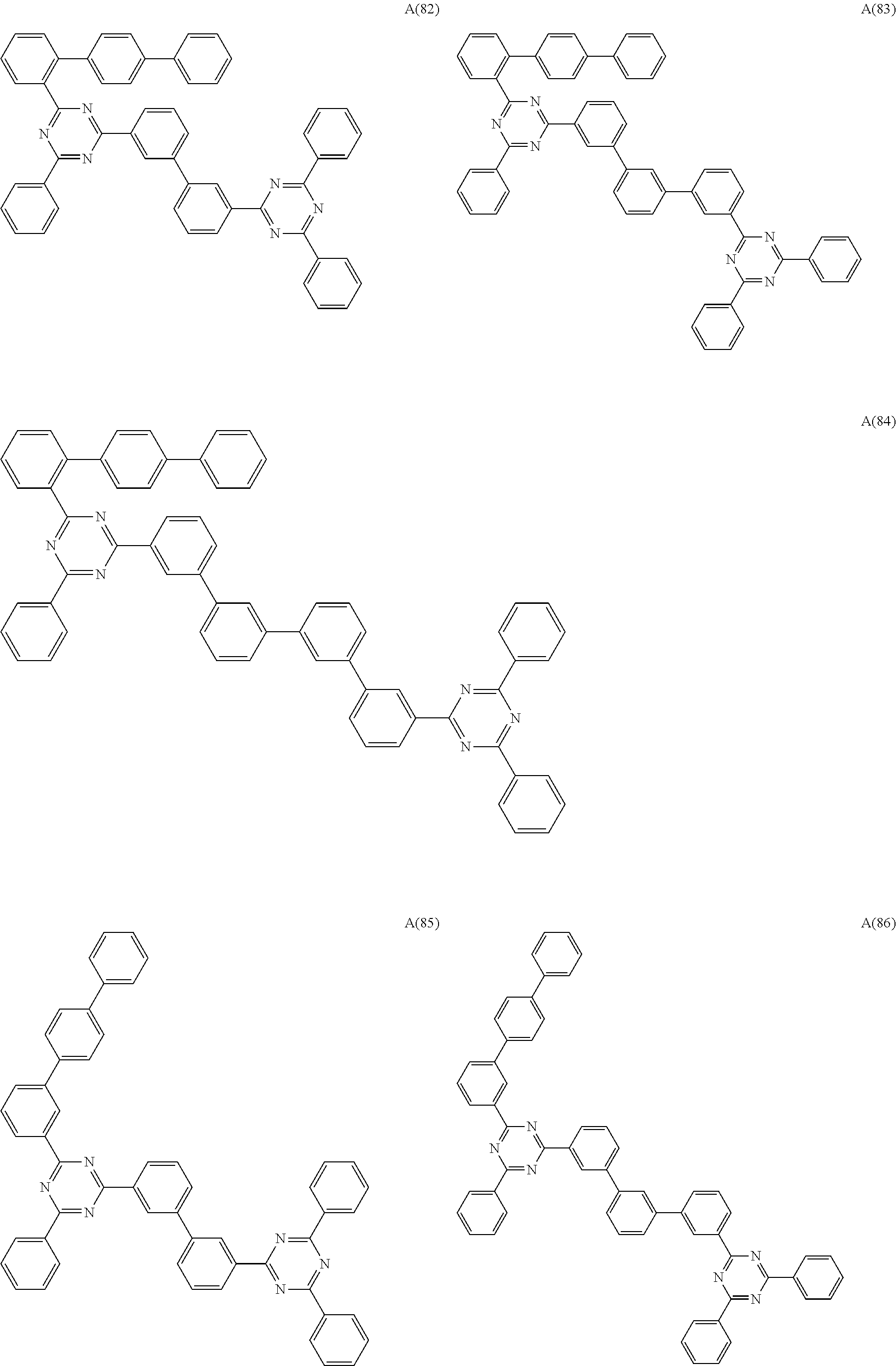

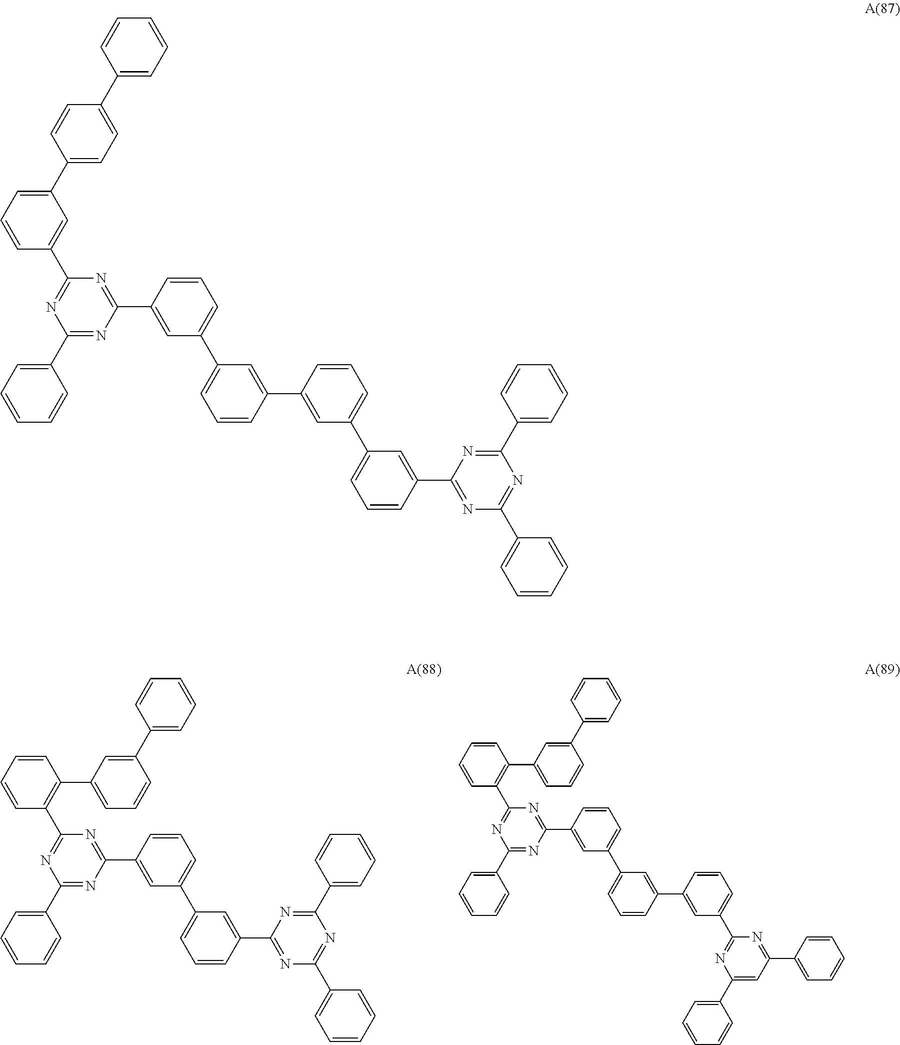 [SMILES: c1ccc(-c2ccc(-c3cccc(-c4nc(-c5ccccc5)nc(-c5cccc(-c6cccc(-c7cccc(-c8cccc(-c9nc(-c%10ccccc%10)nc(-c%10ccccc%10)n9)c8)c7)c6)c5)n4)c3)cc2)cc1 ; c1ccc(-c2cccc(-c3ccccc3-c3nc(-c4ccccc4)nc(-c4cccc(-c5cccc(-c6nc(-c7ccccc7)nc(-c7ccccc7)n6)c5)c4)n3)c2)cc1 ; c1ccc(-c2cccc(-c3ccccc3-c3nc(-c4ccccc4)nc(-c4cccc(-c5cccc(-c6cccc(-c7nc(-c8ccccc8)cc(-c8ccccc8)n7)c6)c5)c4)n3)c2)cc1]

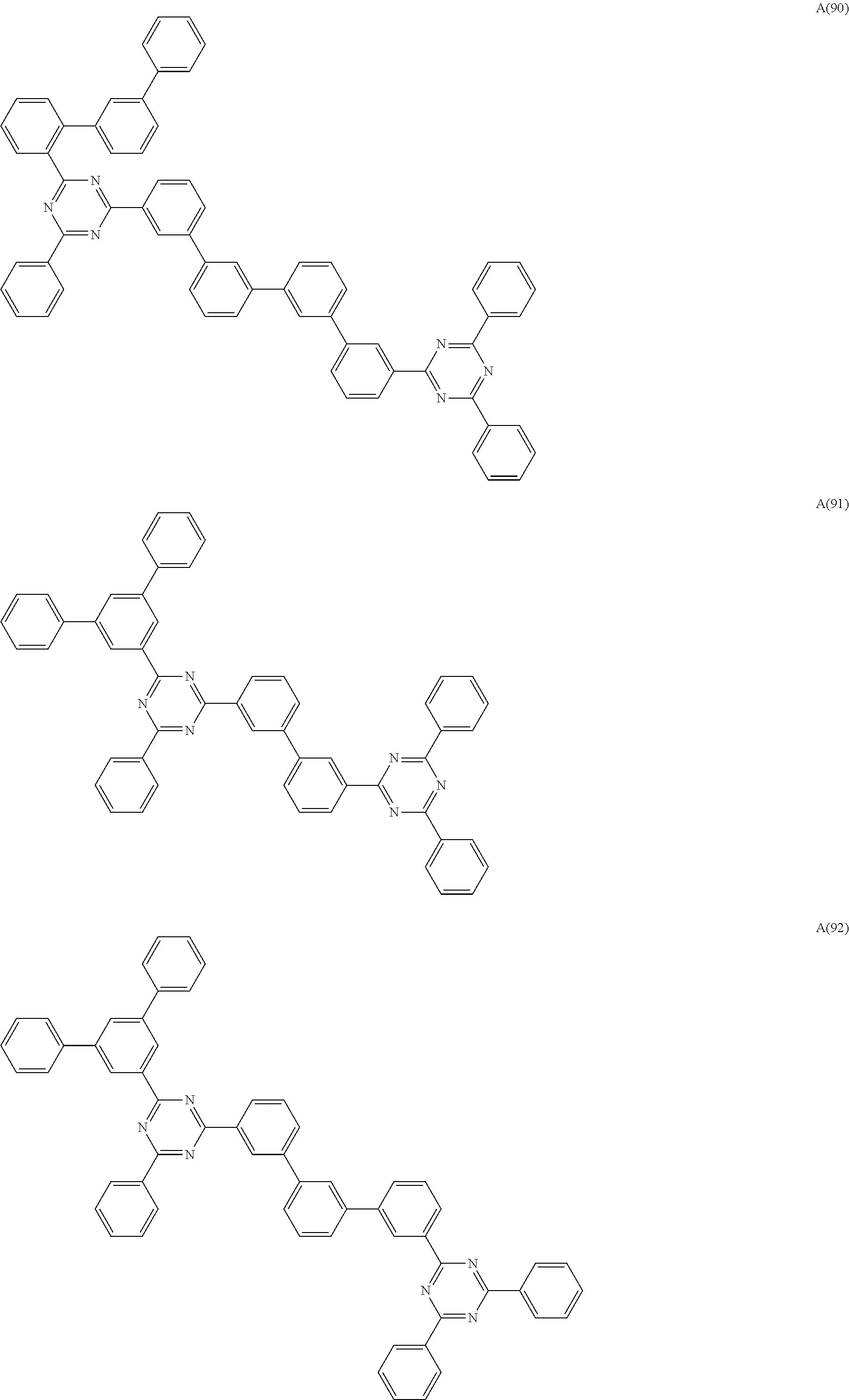 [SMILES: c1ccc(-c2cccc(-c3ccccc3-c3nc(-c4ccccc4)nc(-c4cccc(-c5cccc(-c6cccc(-c7cccc(-c8nc(-c9ccccc9)nc(-c9ccccc9)n8)c7)c6)c5)c4)n3)c2)cc1 ; c1ccc(-c2cc(-c3ccccc3)cc(-c3nc(-c4ccccc4)nc(-c4cccc(-c5cccc(-c6nc(-c7ccccc7)nc(-c7ccccc7)n6)c5)c4)n3)c2)cc1 ; c1ccc(-c2cc(-c3ccccc3)cc(-c3nc(-c4ccccc4)nc(-c4cccc(-c5cccc(-c6cccc(-c7nc(-c8ccccc8)nc(-c8ccccc8)n7)c6)c5)c4)n3)c2)cc1]

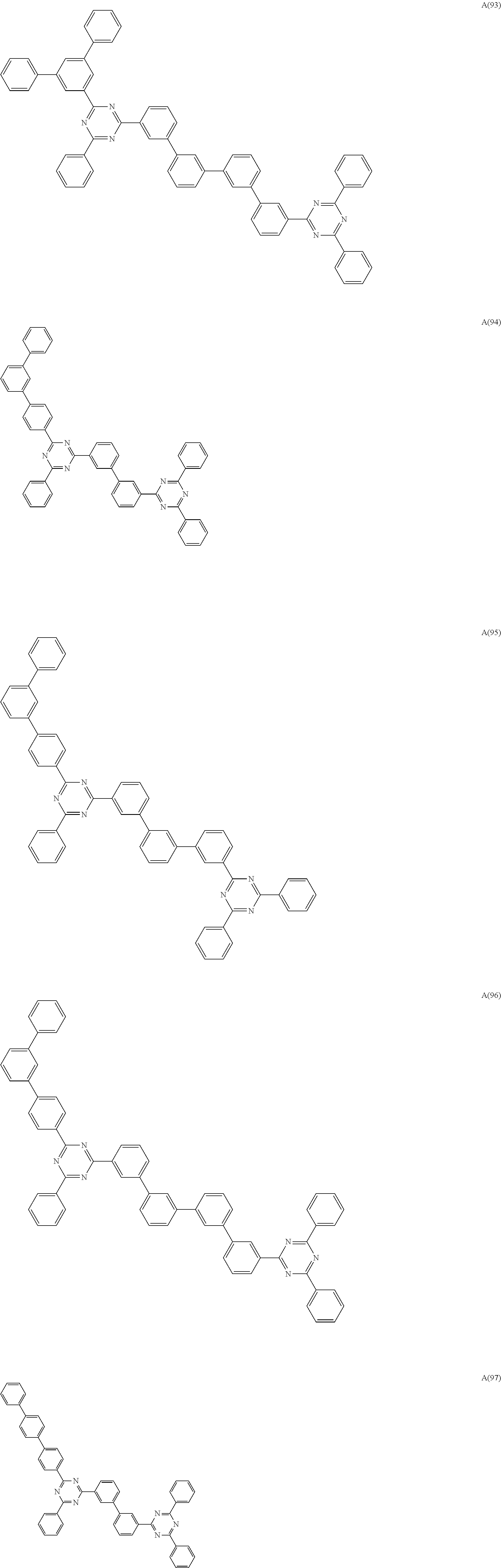

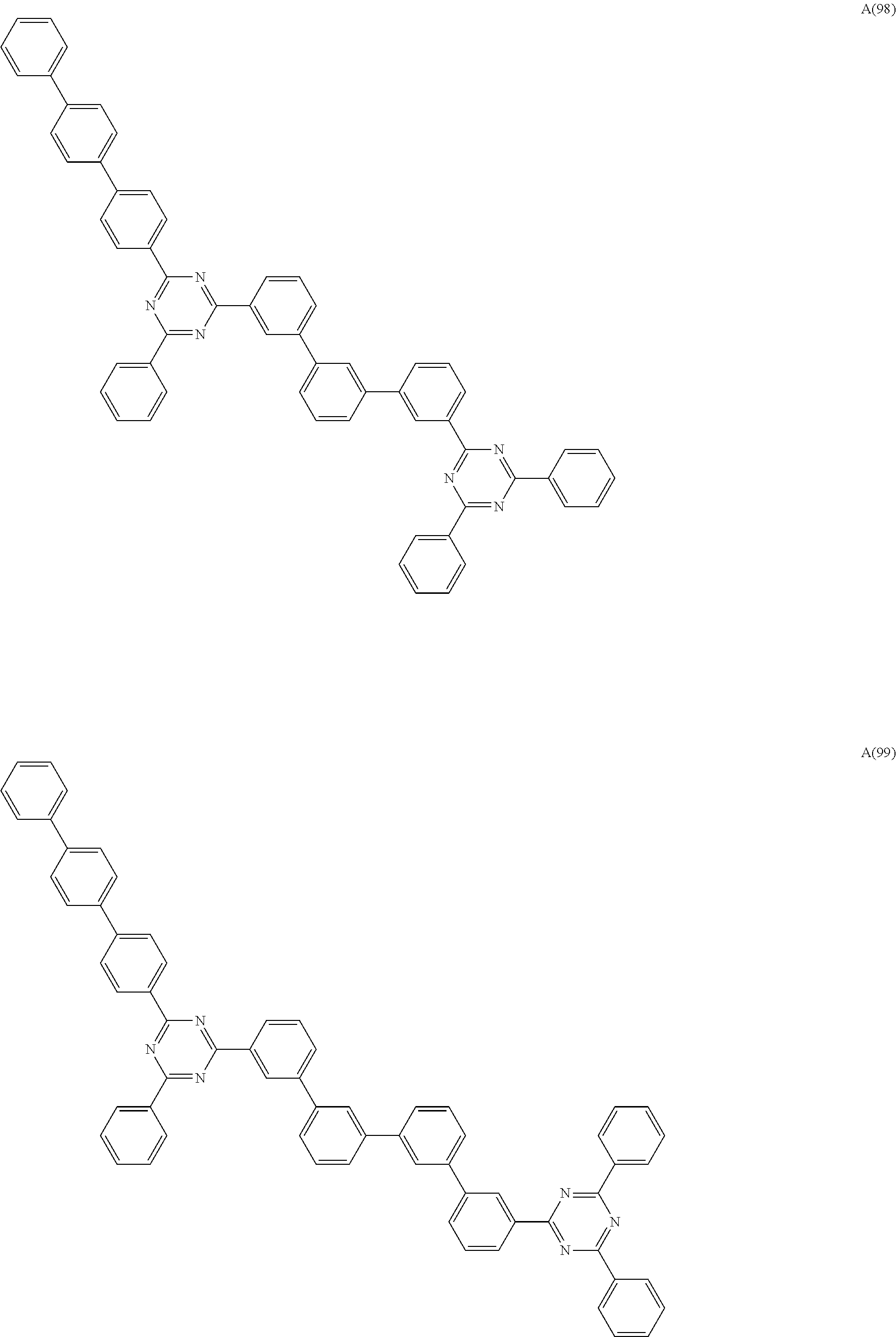 [SMILES: c1ccc(-c2ccc(-c3ccc(-c4nc(-c5ccccc5)nc(-c5cccc(-c6cccc(-c7cccc(-c8nc(-c9ccccc9)nc(-c9ccccc9)n8)c7)c6)c5)n4)cc3)cc2)cc1 ; c1ccc(-c2ccc(-c3ccc(-c4nc(-c5ccccc5)nc(-c5cccc(-c6cccc(-c7cccc(-c8cccc(-c9nc(-c%10ccccc%10)nc(-c%10ccccc%10)n9)c8)c7)c6)c5)n4)cc3)cc2)cc1]

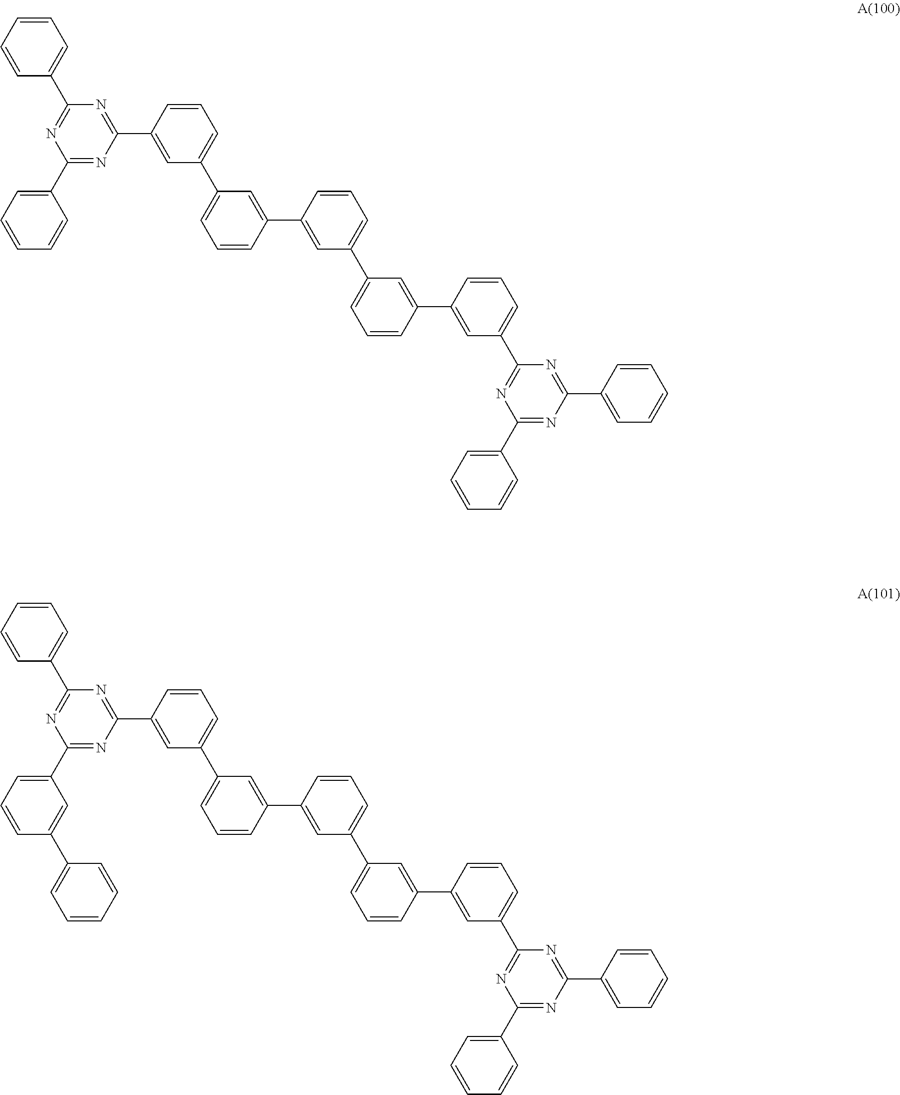 [SMILES: c1ccc(-c2nc(-c3ccccc3)nc(-c3cccc(-c4cccc(-c5cccc(-c6cccc(-c7cccc(-c8nc(-c9ccccc9)nc(-c9ccccc9)n8)c7)c6)c5)c4)c3)n2)cc1 ; c1ccc(-c2cccc(-c3nc(-c4ccccc4)nc(-c4cccc(-c5cccc(-c6cccc(-c7cccc(-c8cccc(-c9nc(-c%10ccccc%10)nc(-c%10ccccc%10)n9)c8)c7)c6)c5)c4)n3)c2)cc1]

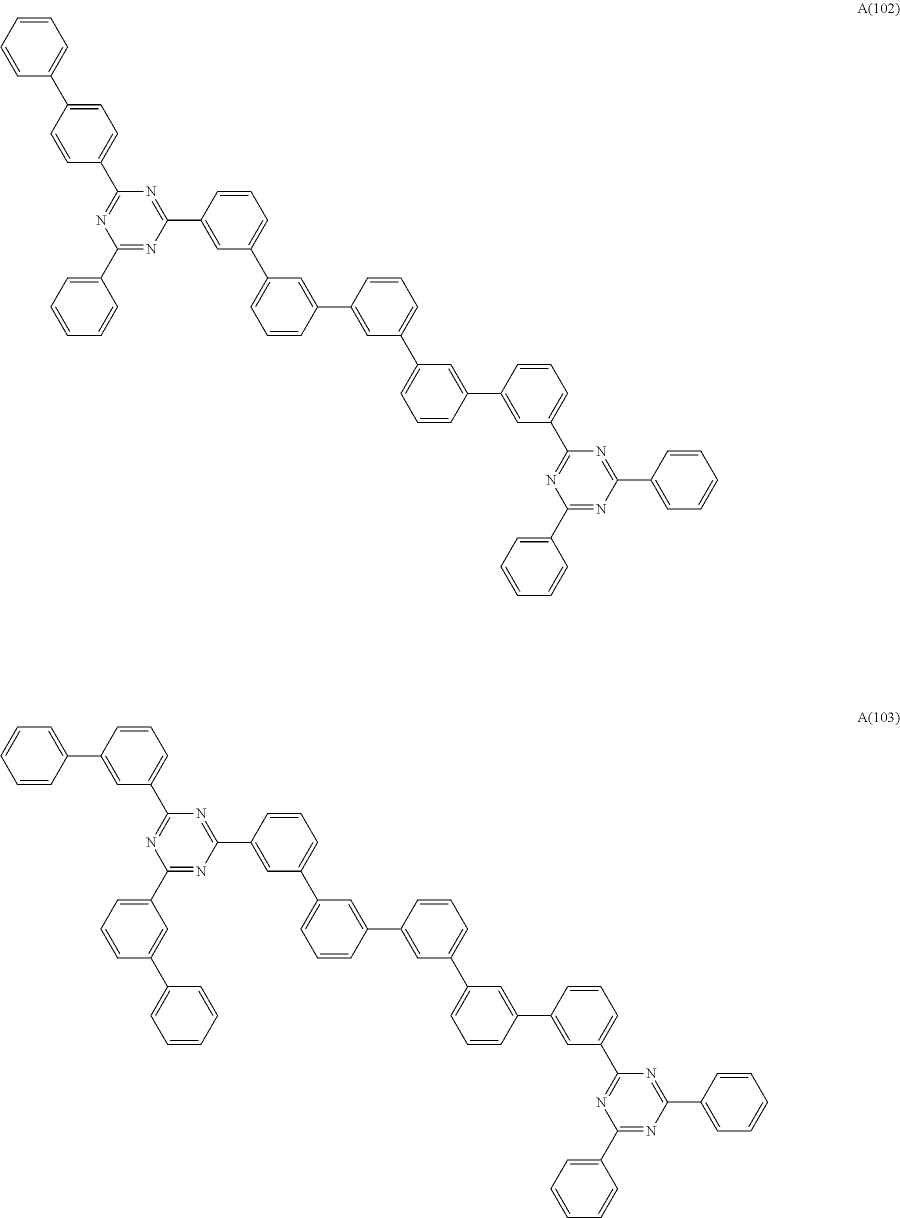 [SMILES: c1ccc(-c2ccc(-c3nc(-c4ccccc4)nc(-c4cccc(-c5cccc(-c6cccc(-c7cccc(-c8cccc(-c9nc(-c%10ccccc%10)nc(-c%10ccccc%10)n9)c8)c7)c6)c5)c4)n3)cc2)cc1 ; c1ccc(-c2cccc(-c3nc(-c4cccc(-c5ccccc5)c4)nc(-c4cccc(-c5cccc(-c6cccc(-c7cccc(-c8cccc(-c9nc(-c%10ccccc%10)nc(-c%10ccccc%10)n9)c8)c7)c6)c5)c4)n3)c2)cc1]

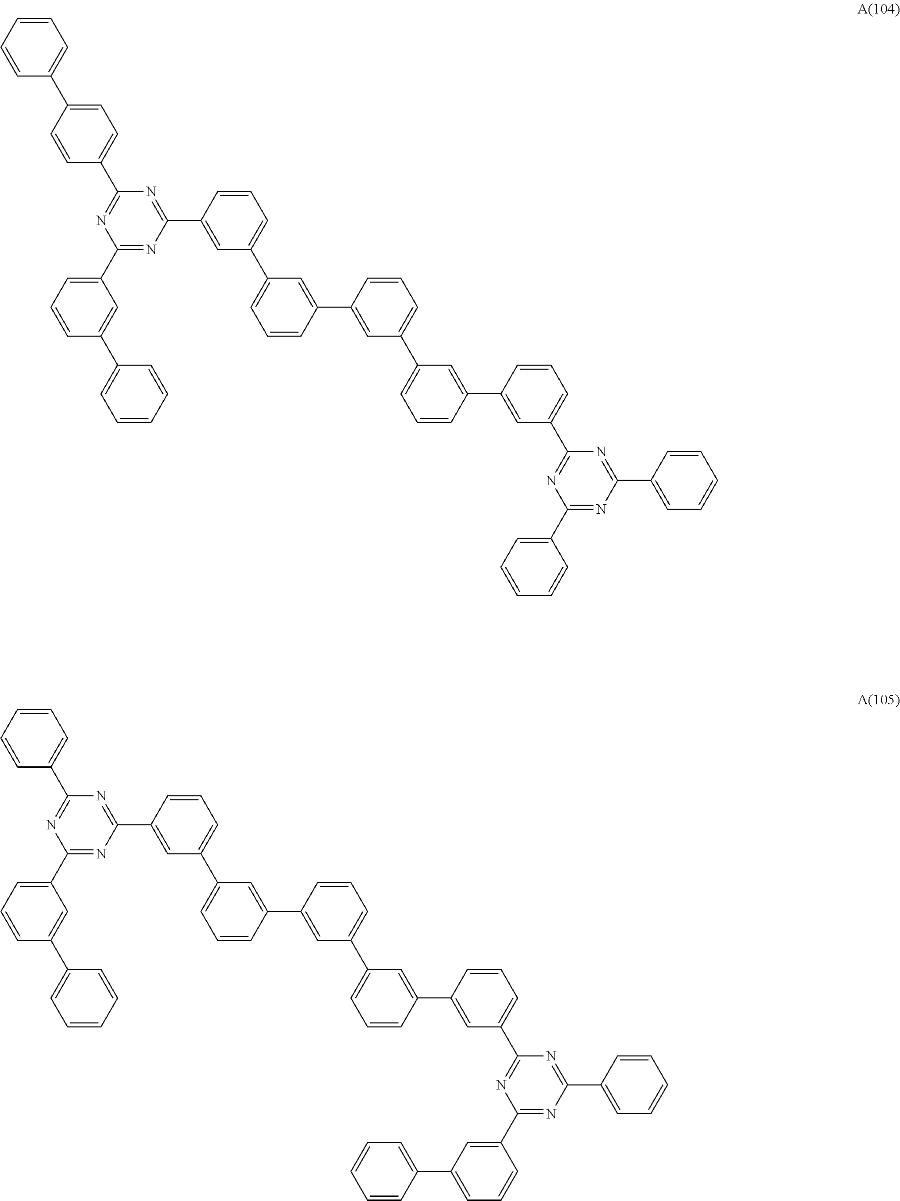 [SMILES: c1ccc(-c2ccc(-c3nc(-c4cccc(-c5ccccc5)c4)nc(-c4cccc(-c5cccc(-c6cccc(-c7cccc(-c8cccc(-c9nc(-c%10ccccc%10)nc(-c%10ccccc%10)n9)c8)c7)c6)c5)c4)n3)cc2)cc1 ; c1ccc(-c2cccc(-c3nc(-c4ccccc4)nc(-c4cccc(-c5cccc(-c6cccc(-c7cccc(-c8cccc(-c9nc(-c%10ccccc%10)nc(-c%10cccc(-c%11ccccc%11)c%10)n9)c8)c7)c6)c5)c4)n3)c2)cc1]

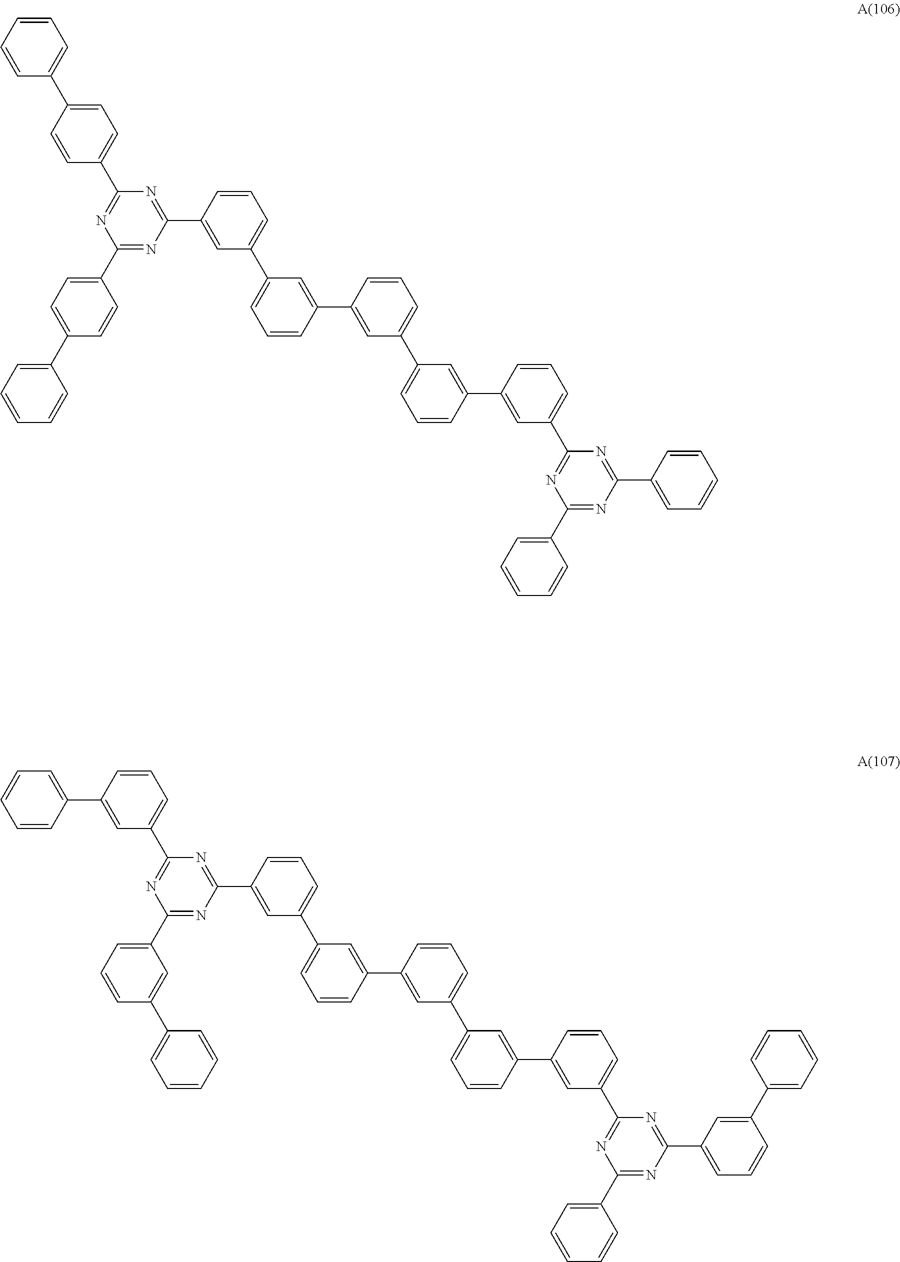 [SMILES: c1ccc(-c2ccc(-c3nc(-c4ccc(-c5ccccc5)cc4)nc(-c4cccc(-c5cccc(-c6cccc(-c7cccc(-c8cccc(-c9nc(-c%10ccccc%10)nc(-c%10ccccc%10)n9)c8)c7)c6)c5)c4)n3)cc2)cc1 ; c1ccc(-c2cccc(-c3nc(-c4ccccc4)nc(-c4cccc(-c5cccc(-c6cccc(-c7cccc(-c8cccc(-c9nc(-c%10cccc(-c%11ccccc%11)c%10)nc(-c%10cccc(-c%11ccccc%11)c%10)n9)c8)c7)c6)c5)c4)n3)c2)cc1]

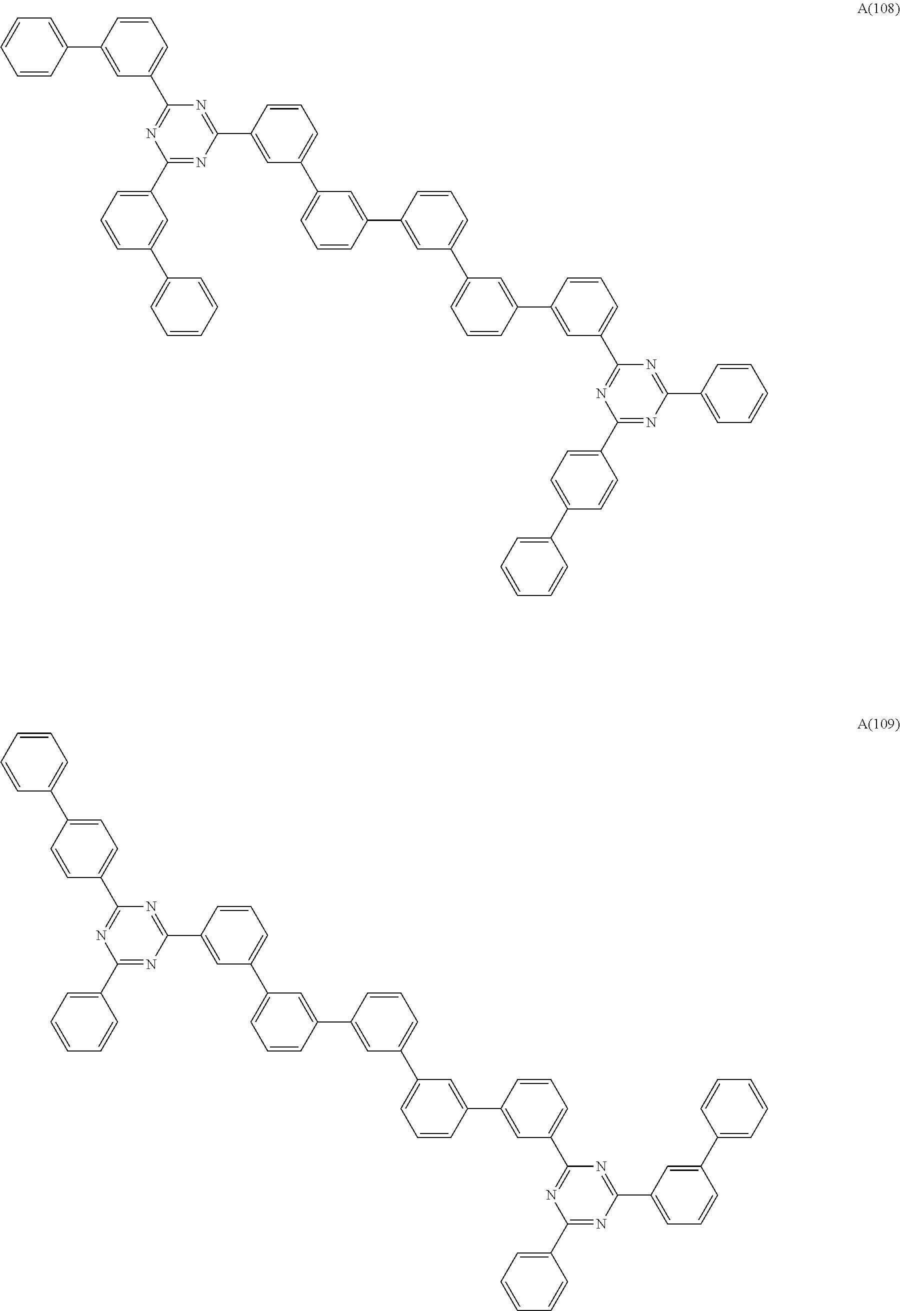 [SMILES: c1ccc(-c2ccc(-c3nc(-c4ccccc4)nc(-c4cccc(-c5cccc(-c6cccc(-c7cccc(-c8cccc(-c9nc(-c%10cccc(-c%11ccccc%11)c%10)nc(-c%10cccc(-c%11ccccc%11)c%10)n9)c8)c7)c6)c5)c4)n3)cc2)cc1 ; c1ccc(-c2ccc(-c3nc(-c4ccccc4)nc(-c4cccc(-c5cccc(-c6cccc(-c7cccc(-c8cccc(-c9nc(-c%10ccccc%10)nc(-c%10cccc(-c%11ccccc%11)c%10)n9)c8)c7)c6)c5)c4)n3)cc2)cc1]

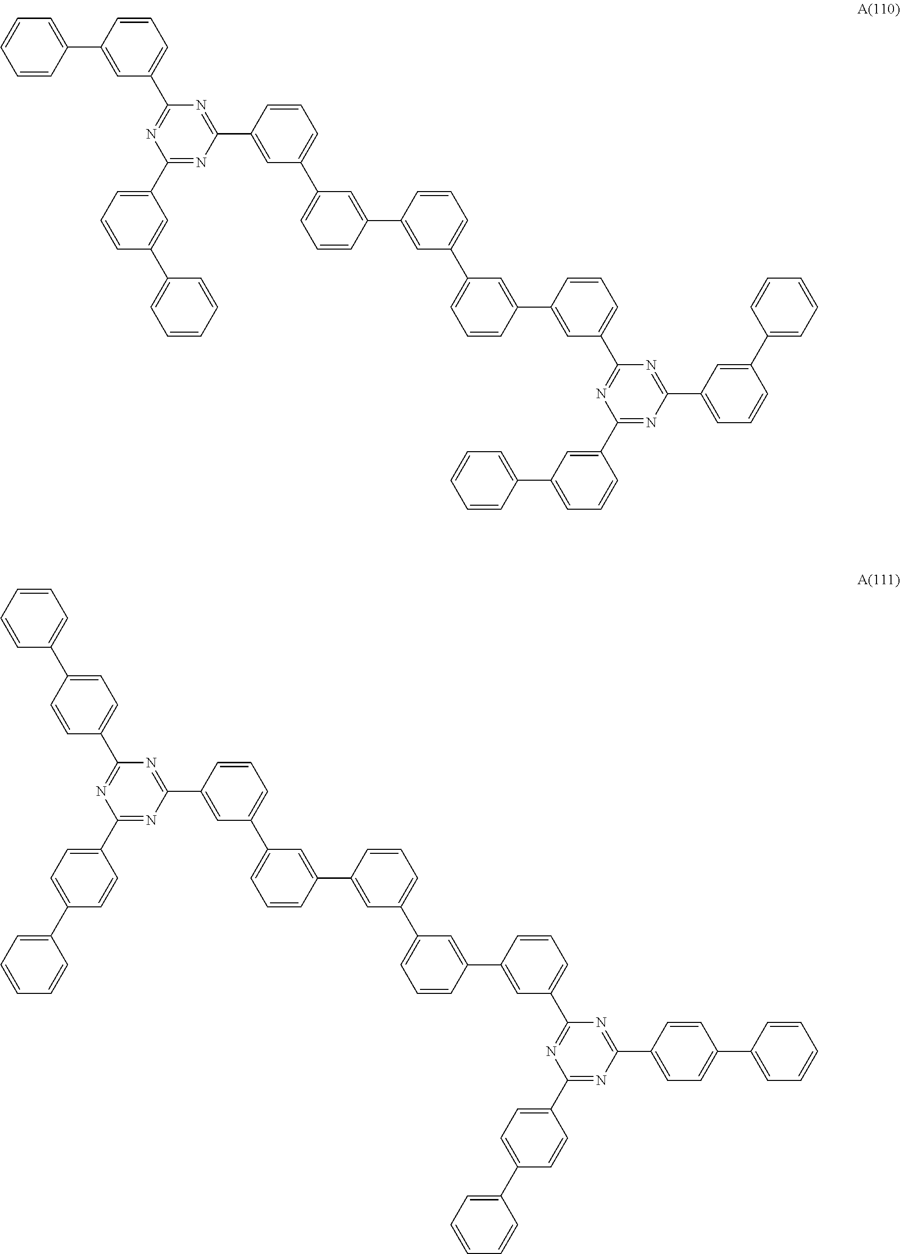 [SMILES: c1ccc(-c2cccc(-c3nc(-c4cccc(-c5ccccc5)c4)nc(-c4cccc(-c5cccc(-c6cccc(-c7cccc(-c8cccc(-c9nc(-c%10cccc(-c%11ccccc%11)c%10)nc(-c%10cccc(-c%11ccccc%11)c%10)n9)c8)c7)c6)c5)c4)n3)c2)cc1 ; c1ccc(-c2ccc(-c3nc(-c4ccc(-c5ccccc5)cc4)nc(-c4cccc(-c5cccc(-c6cccc(-c7cccc(-c8cccc(-c9nc(-c%10ccc(-c%11ccccc%11)cc%10)nc(-c%10ccc(-c%11ccccc%11)cc%10)n9)c8)c7)c6)c5)c4)n3)cc2)cc1]

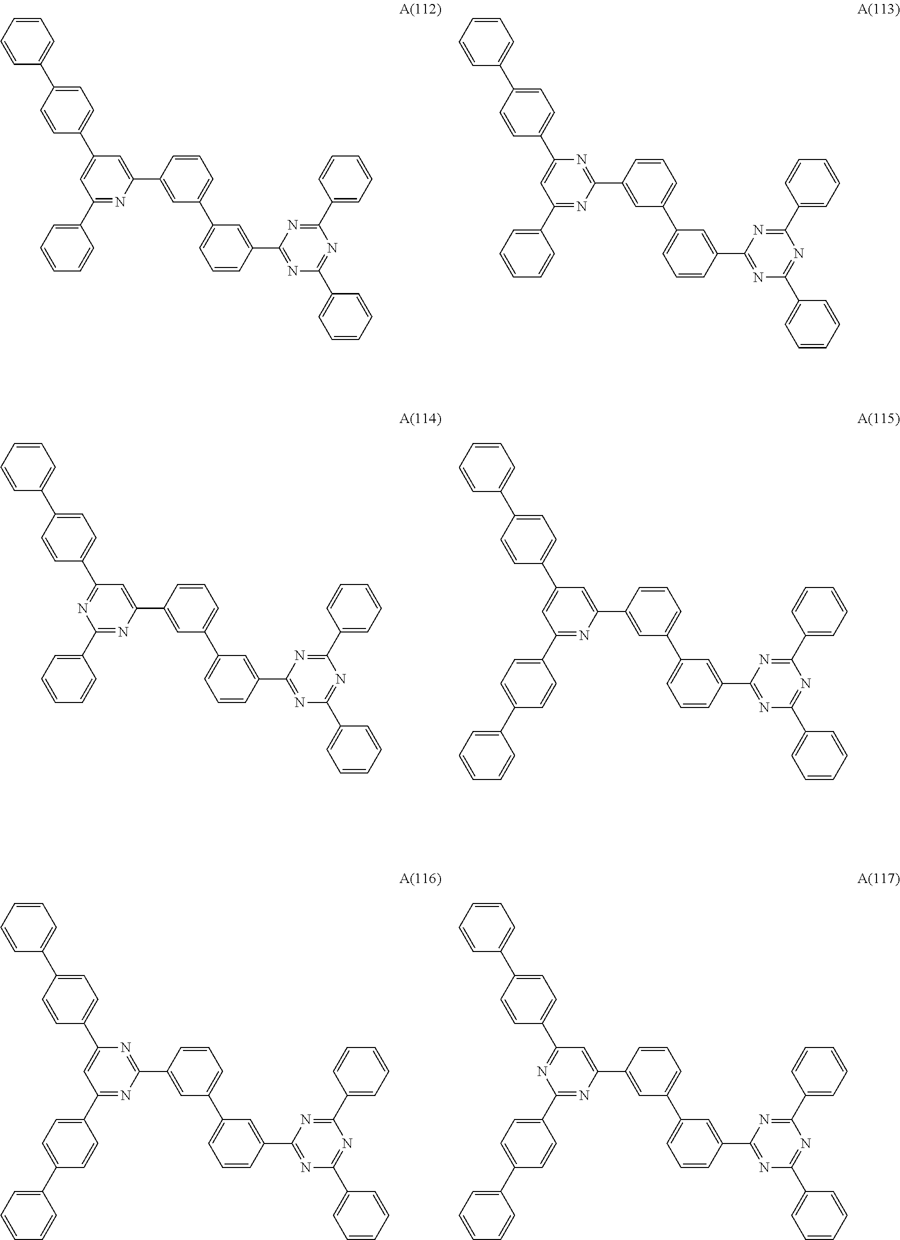

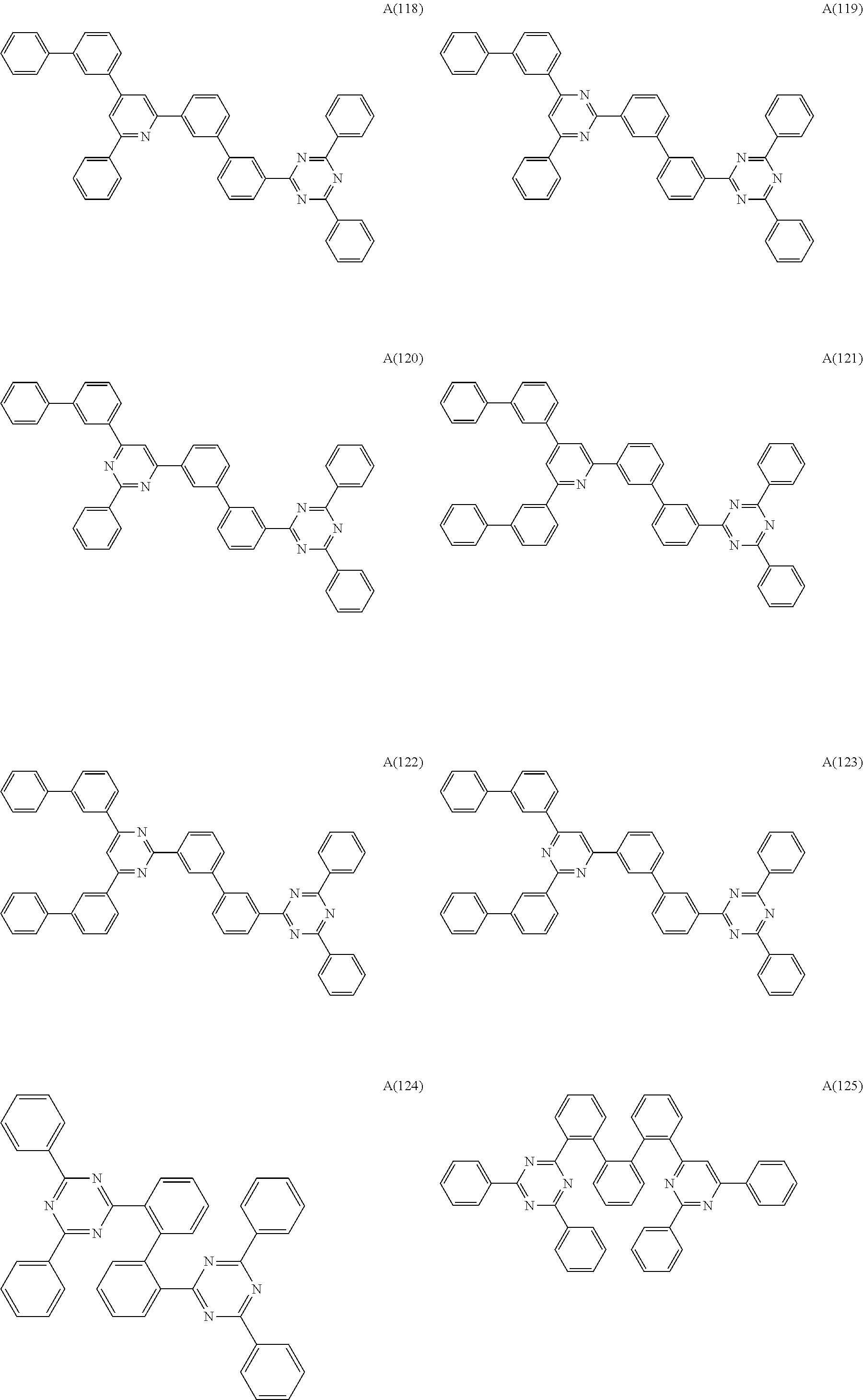

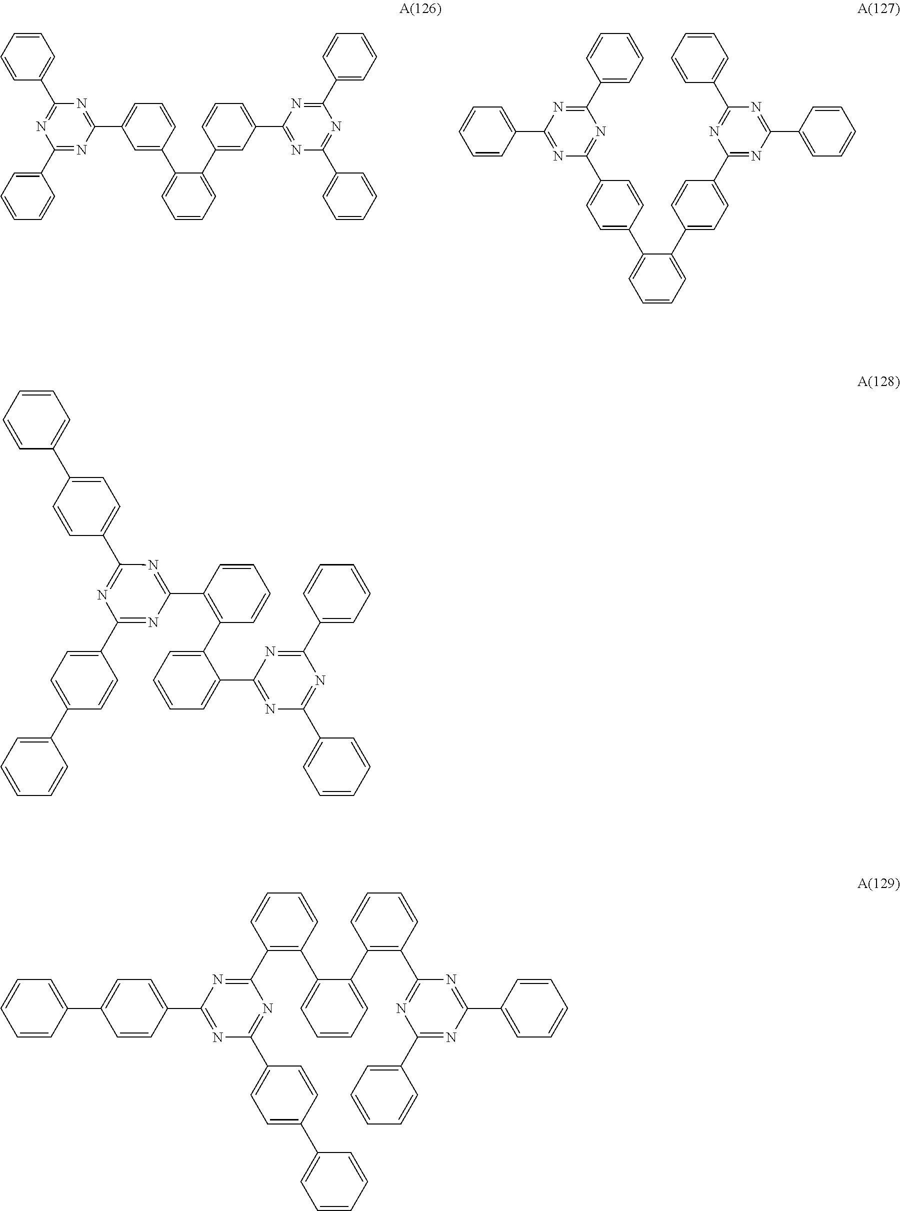 [SMILES: c1ccc(-c2nc(-c3ccccc3)nc(-c3cccc(-c4ccccc4-c4cccc(-c5nc(-c6ccccc6)nc(-c6ccccc6)n5)c4)c3)n2)cc1 ; c1ccc(-c2nc(-c3ccccc3)nc(-c3ccc(-c4ccccc4-c4ccc(-c5nc(-c6ccccc6)nc(-c6ccccc6)n5)cc4)cc3)n2)cc1 ; c1ccc(-c2ccc(-c3nc(-c4ccc(-c5ccccc5)cc4)nc(-c4ccccc4-c4ccccc4-c4nc(-c5ccccc5)nc(-c5ccccc5)n4)n3)cc2)cc1 ; c1ccc(-c2ccc(-c3nc(-c4ccc(-c5ccccc5)cc4)nc(-c4ccccc4-c4ccccc4-c4ccccc4-c4nc(-c5ccccc5)nc(-c5ccccc5)n4)n3)cc2)cc1]

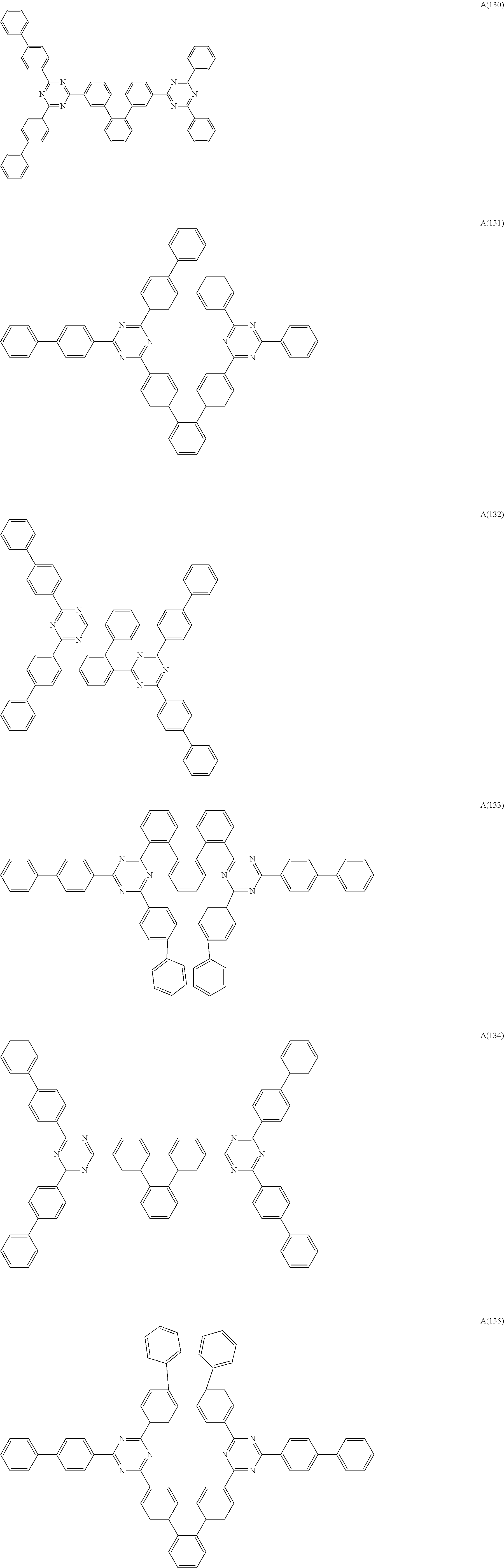

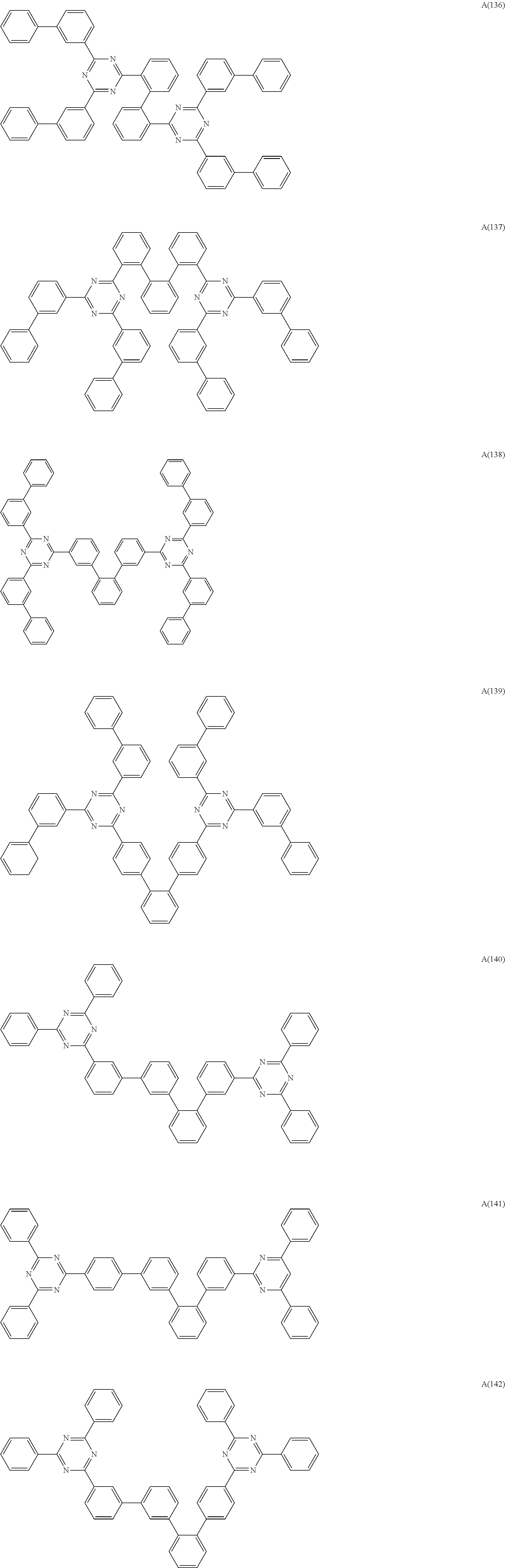

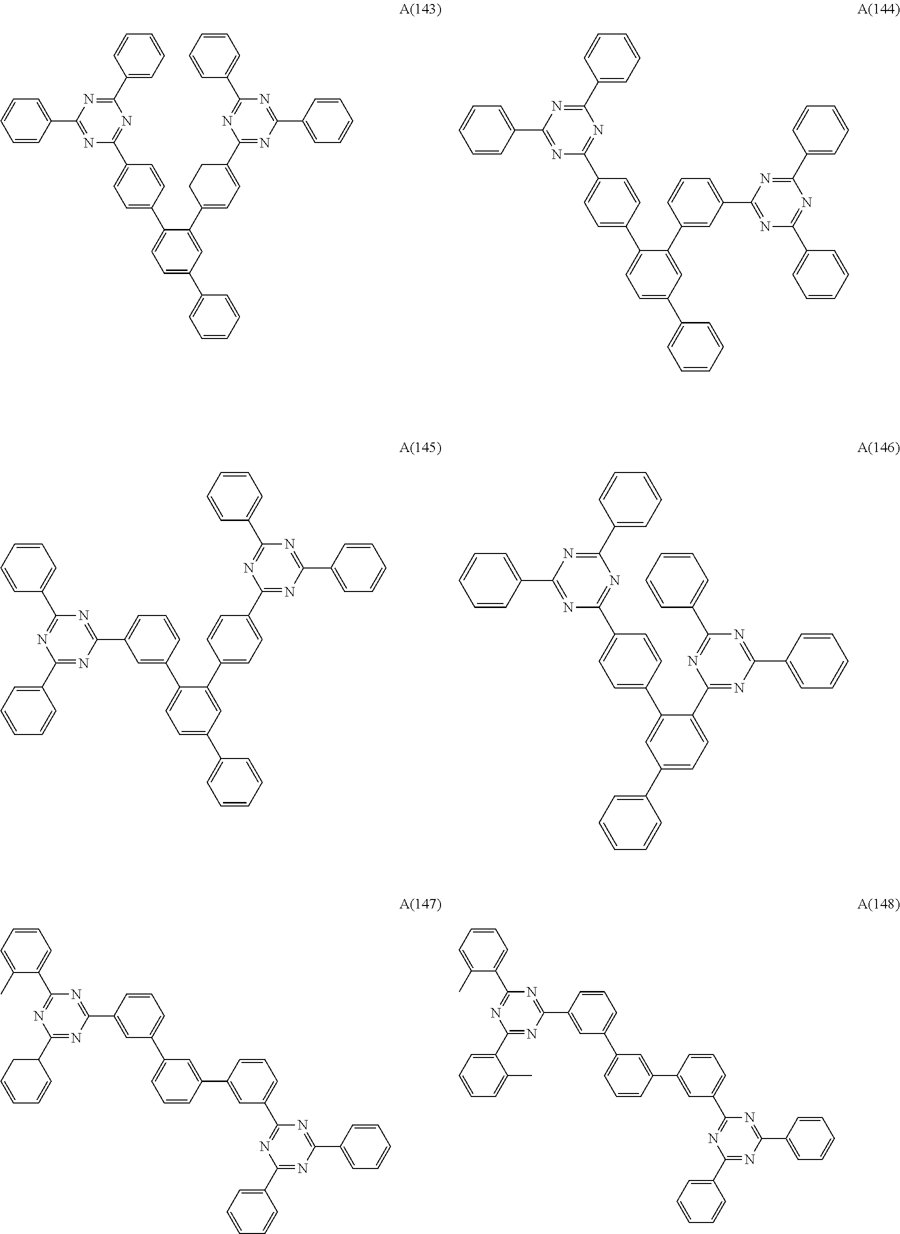

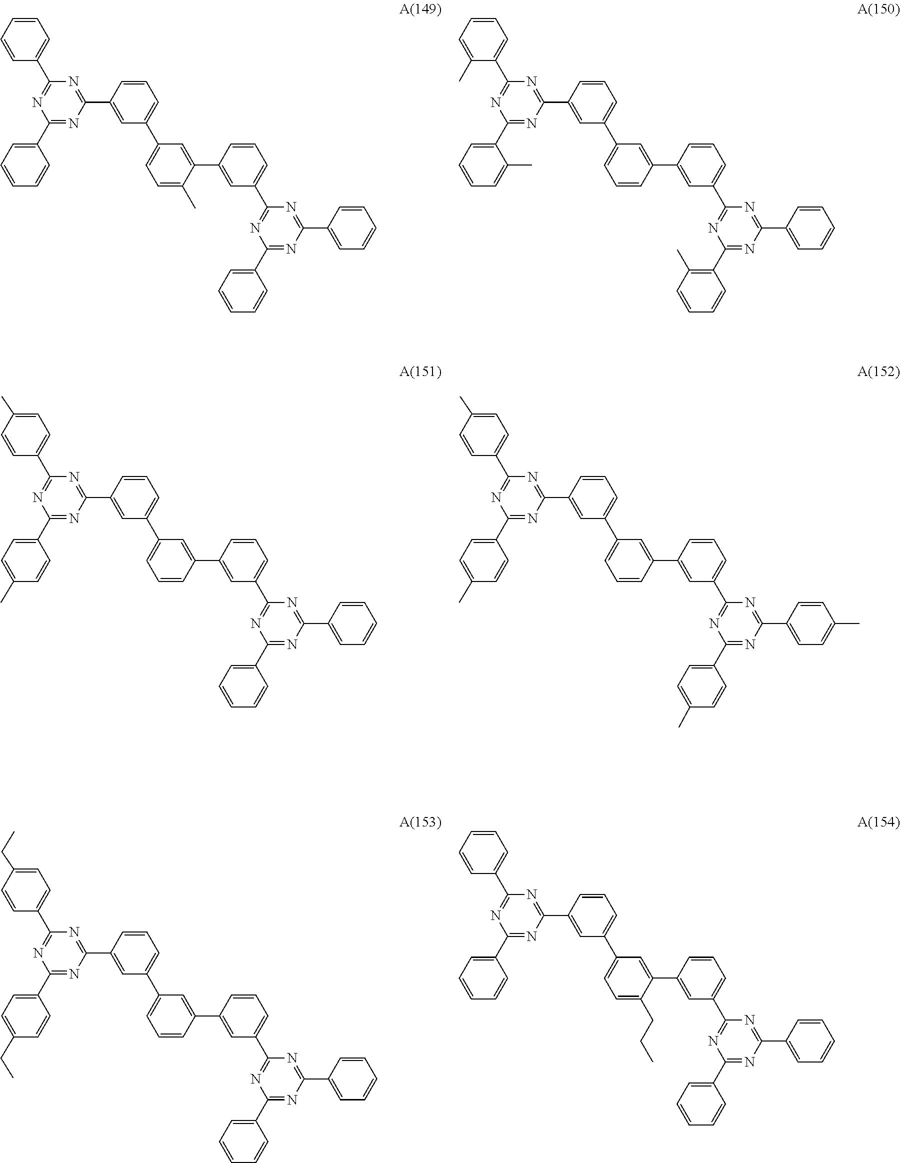

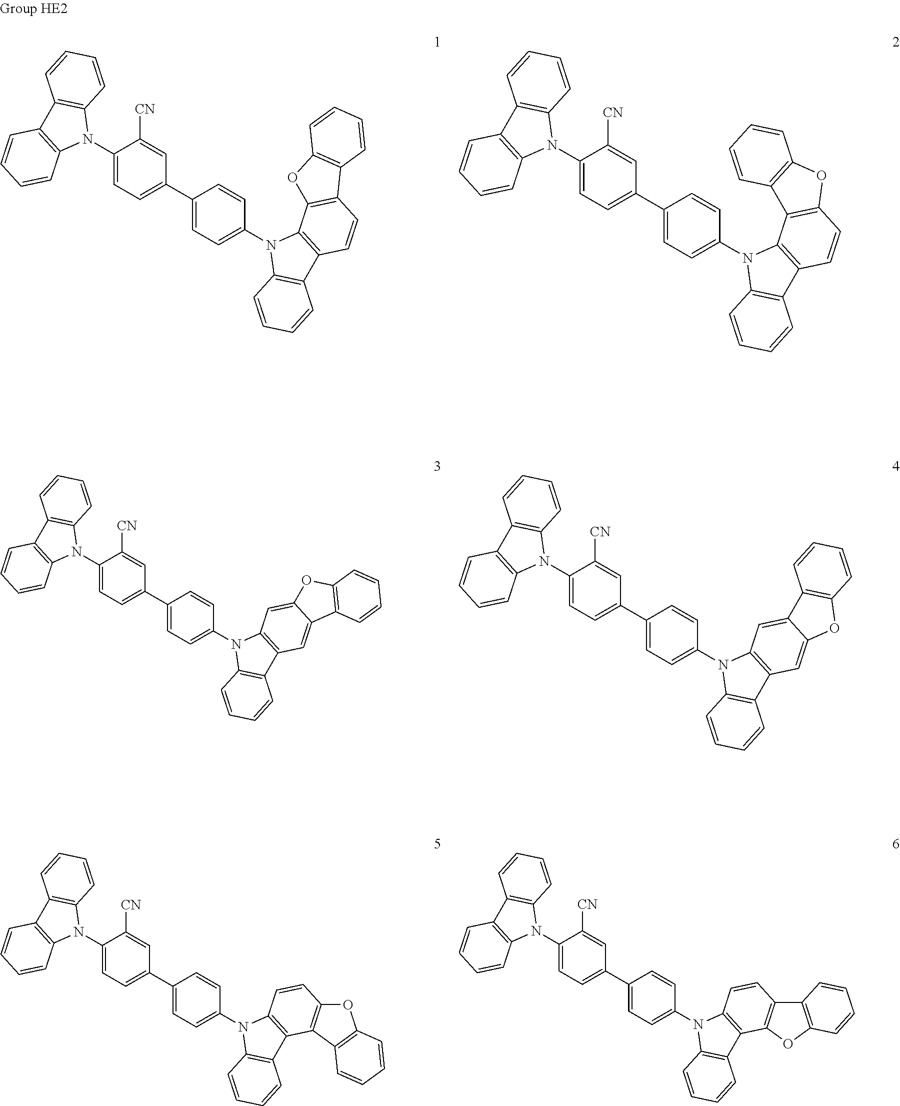 [SMILES: N#Cc1cc(-c2ccc(-n3c4ccccc4c4ccc5c6ccccc6oc5c43)cc2)ccc1-n1c2ccccc2c2ccccc21 ; N#Cc1cc(-c2ccc(-n3c4ccccc4c4ccc5oc6ccccc6c5c43)cc2)ccc1-n1c2ccccc2c2ccccc21 ; N#Cc1cc(-c2ccc(-n3c4ccccc4c4cc5c(cc43)oc3ccccc35)cc2)ccc1-n1c2ccccc2c2ccccc21 ; N#Cc1cc(-c2ccc(-n3c4ccccc4c4cc5oc6ccccc6c5cc43)cc2)ccc1-n1c2ccccc2c2ccccc21 ; N#Cc1cc(-c2ccc(-n3c4ccccc4c4c5c(ccc43)oc3ccccc35)cc2)ccc1-n1c2ccccc2c2ccccc21 ; N#Cc1cc(-c2ccc(-n3c4ccccc4c4c5oc6ccccc6c5ccc43)cc2)ccc1-n1c2ccccc2c2ccccc21]

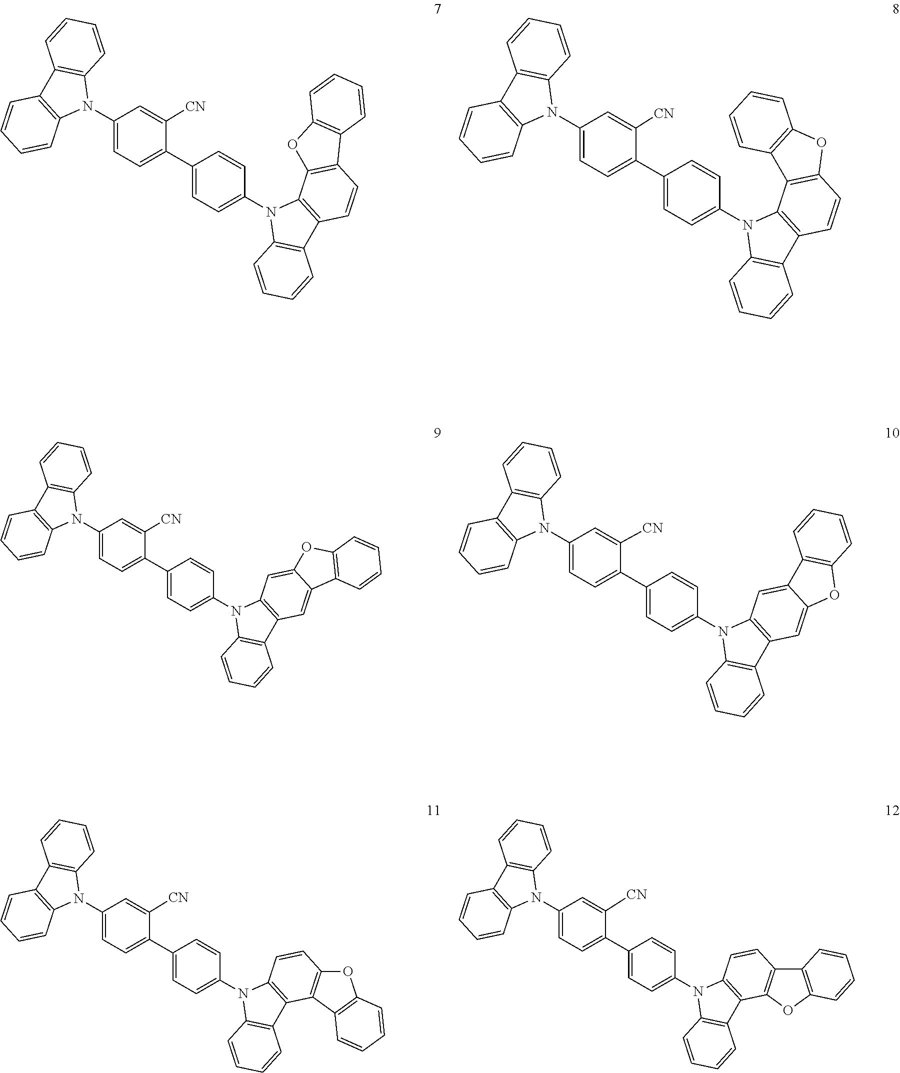 [SMILES: N#Cc1cc(-n2c3ccccc3c3ccccc32)ccc1-c1ccc(-n2c3ccccc3c3ccc4c5ccccc5oc4c32)cc1 ; N#Cc1cc(-n2c3ccccc3c3ccccc32)ccc1-c1ccc(-n2c3ccccc3c3ccc4oc5ccccc5c4c32)cc1 ; N#Cc1cc(-n2c3ccccc3c3ccccc32)ccc1-c1ccc(-n2c3ccccc3c3cc4c(cc32)oc2ccccc24)cc1 ; N#Cc1cc(-n2c3ccccc3c3ccccc32)ccc1-c1ccc(-n2c3ccccc3c3cc4oc5ccccc5c4cc32)cc1 ; N#Cc1cc(-n2c3ccccc3c3ccccc32)ccc1-c1ccc(-n2c3ccccc3c3c4c(ccc32)oc2ccccc24)cc1 ; N#Cc1cc(-n2c3ccccc3c3ccccc32)ccc1-c1ccc(-n2c3ccccc3c3c4oc5ccccc5c4ccc32)cc1]

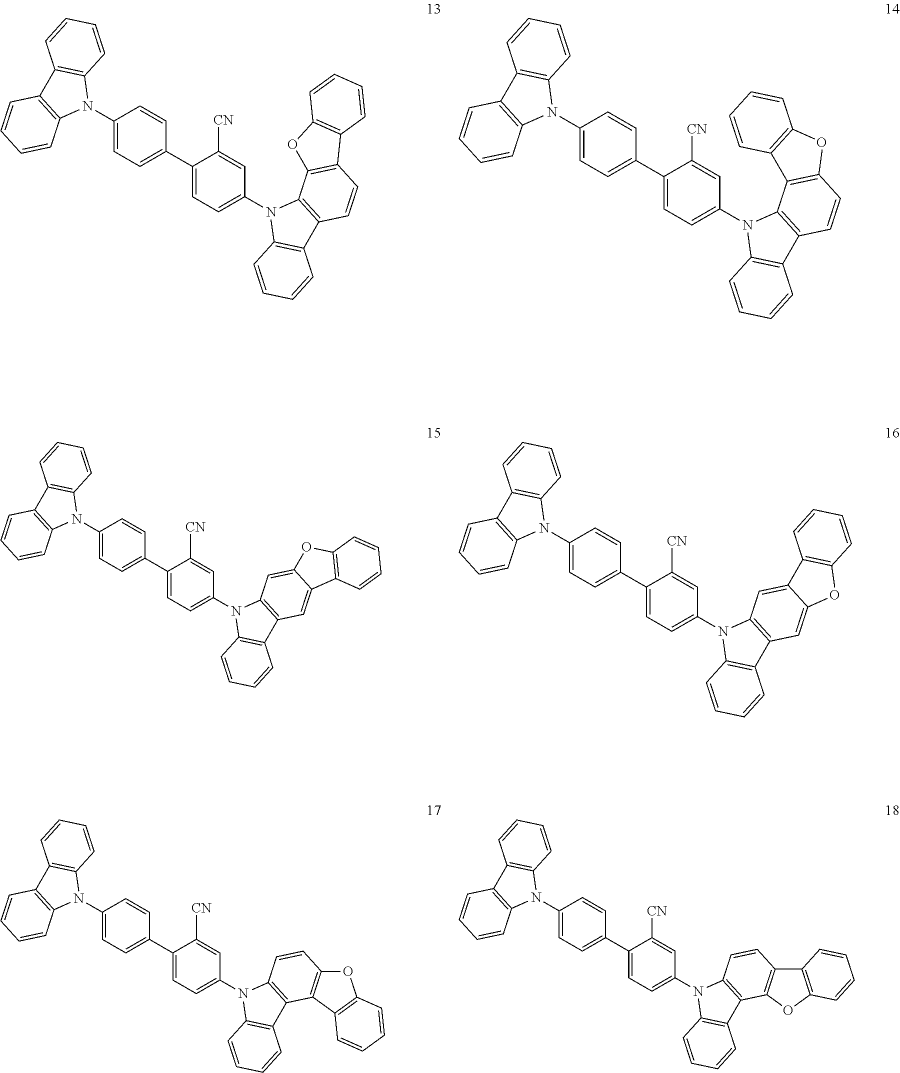 [SMILES: N#Cc1cc(-n2c3ccccc3c3ccc4c5ccccc5oc4c32)ccc1-c1ccc(-n2c3ccccc3c3ccccc32)cc1 ; N#Cc1cc(-n2c3ccccc3c3ccc4oc5ccccc5c4c32)ccc1-c1ccc(-n2c3ccccc3c3ccccc32)cc1 ; N#Cc1cc(-n2c3ccccc3c3cc4c(cc32)oc2ccccc24)ccc1-c1ccc(-n2c3ccccc3c3ccccc32)cc1 ; N#Cc1cc(-n2c3ccccc3c3cc4oc5ccccc5c4cc32)ccc1-c1ccc(-n2c3ccccc3c3ccccc32)cc1 ; N#Cc1cc(-n2c3ccccc3c3c4c(ccc32)oc2ccccc24)ccc1-c1ccc(-n2c3ccccc3c3ccccc32)cc1 ; N#Cc1cc(-n2c3ccccc3c3c4oc5ccccc5c4ccc32)ccc1-c1ccc(-n2c3ccccc3c3ccccc32)cc1]

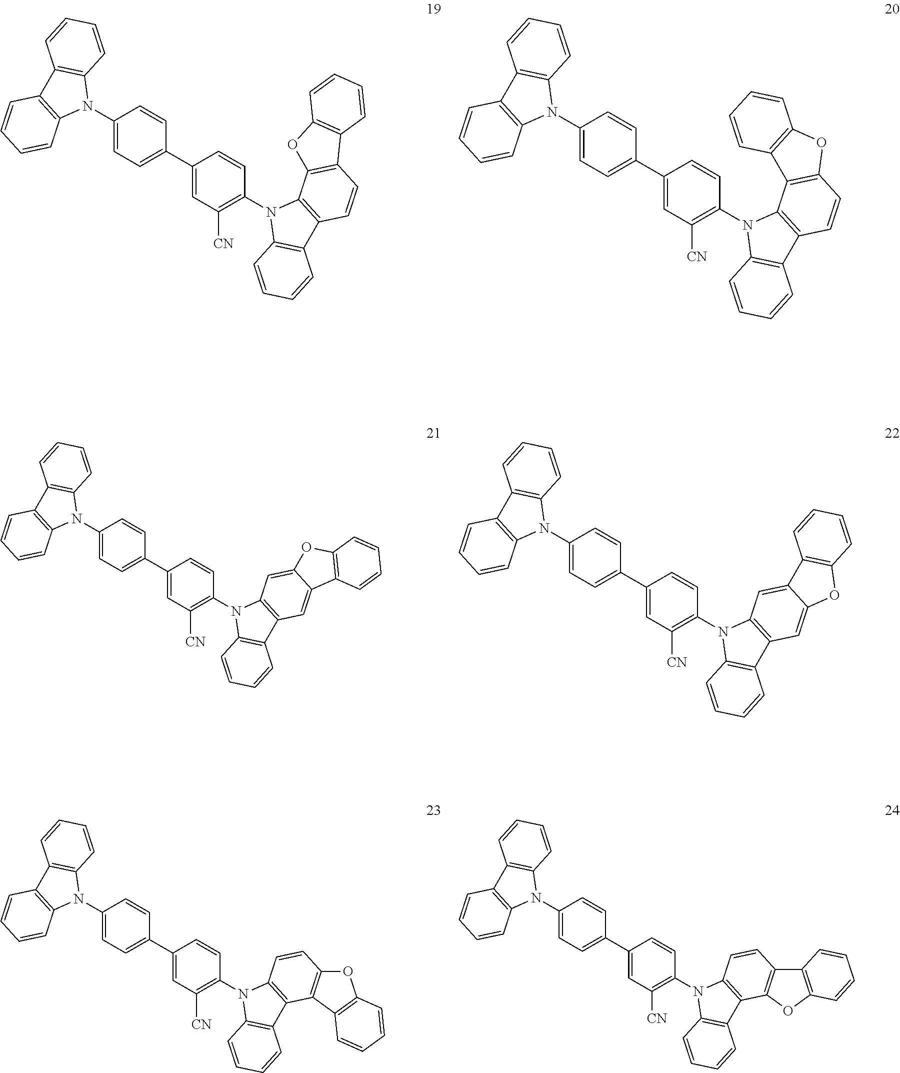 [SMILES: N#Cc1cc(-c2ccc(-n3c4ccccc4c4ccccc43)cc2)ccc1-n1c2ccccc2c2ccc3c4ccccc4oc3c21 ; N#Cc1cc(-c2ccc(-n3c4ccccc4c4ccccc43)cc2)ccc1-n1c2ccccc2c2ccc3oc4ccccc4c3c21 ; N#Cc1cc(-c2ccc(-n3c4ccccc4c4ccccc43)cc2)ccc1-n1c2ccccc2c2cc3c(cc21)oc1ccccc13 ; N#Cc1cc(-c2ccc(-n3c4ccccc4c4ccccc43)cc2)ccc1-n1c2ccccc2c2cc3oc4ccccc4c3cc21 ; N#Cc1cc(-c2ccc(-n3c4ccccc4c4ccccc43)cc2)ccc1-n1c2ccccc2c2c3c(ccc21)oc1ccccc13 ; N#Cc1cc(-c2ccc(-n3c4ccccc4c4ccccc43)cc2)ccc1-n1c2ccccc2c2c3oc4ccccc4c3ccc21]

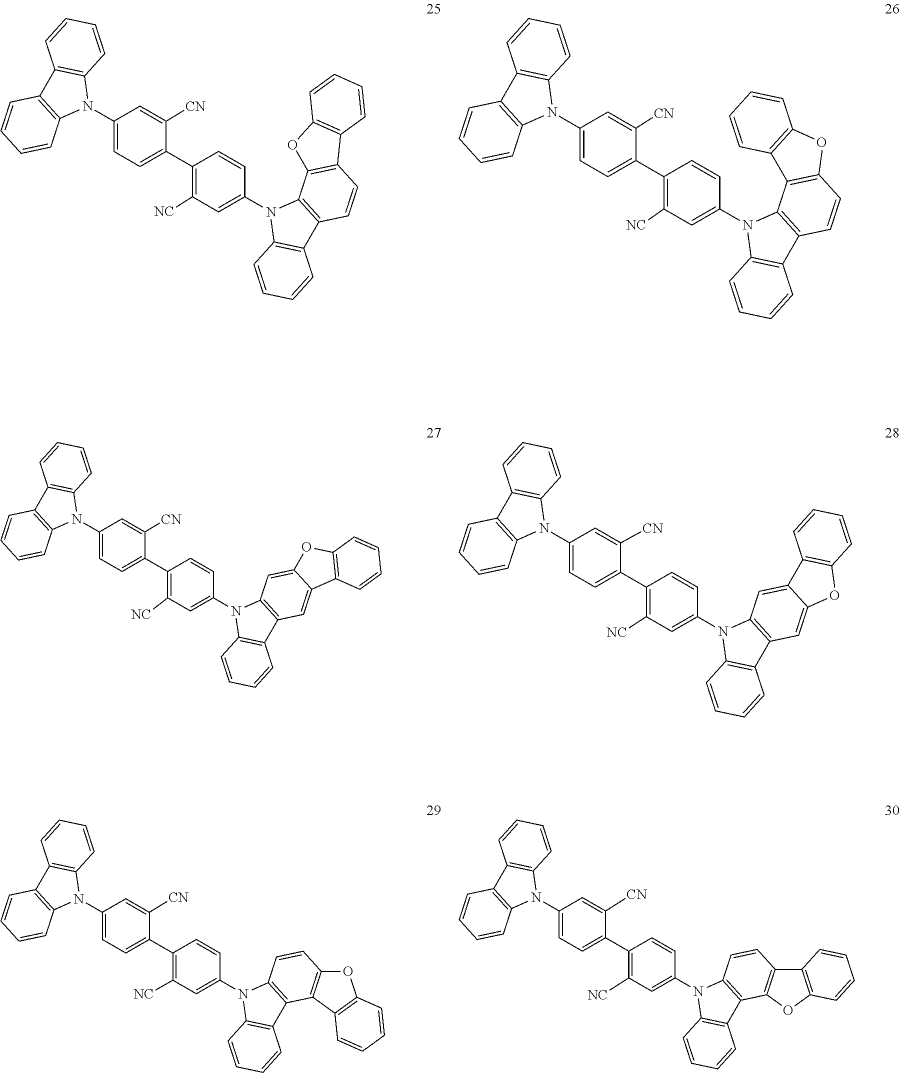 [SMILES: N#Cc1cc(-n2c3ccccc3c3ccccc32)ccc1-c1ccc(-n2c3ccccc3c3ccc4c5ccccc5oc4c32)cc1C#N ; N#Cc1cc(-n2c3ccccc3c3ccccc32)ccc1-c1ccc(-n2c3ccccc3c3ccc4oc5ccccc5c4c32)cc1C#N ; N#Cc1cc(-n2c3ccccc3c3ccccc32)ccc1-c1ccc(-n2c3ccccc3c3cc4c(cc32)oc2ccccc24)cc1C#N ; N#Cc1cc(-n2c3ccccc3c3ccccc32)ccc1-c1ccc(-n2c3ccccc3c3cc4oc5ccccc5c4cc32)cc1C#N ; N#Cc1cc(-n2c3ccccc3c3ccccc32)ccc1-c1ccc(-n2c3ccccc3c3c4c(ccc32)oc2ccccc24)cc1C#N ; N#Cc1cc(-n2c3ccccc3c3ccccc32)ccc1-c1ccc(-n2c3ccccc3c3c4oc5ccccc5c4ccc32)cc1C#N]

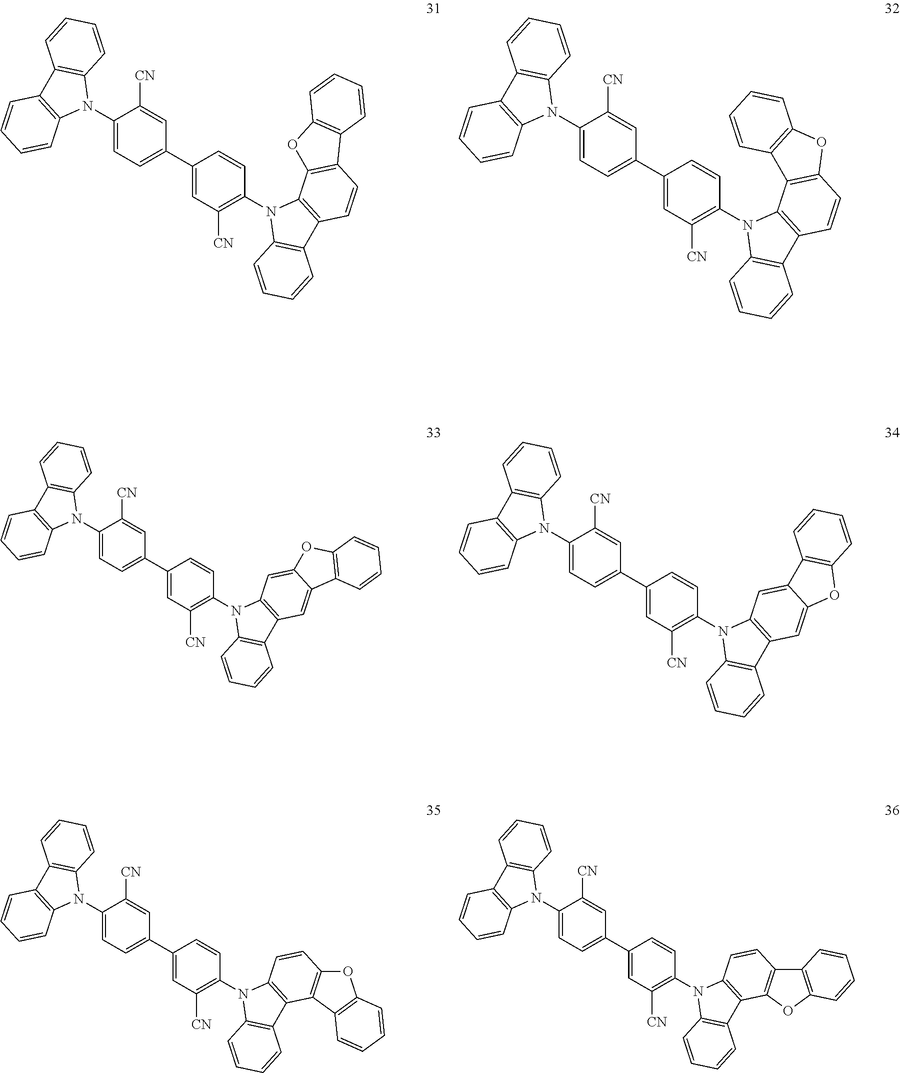 [SMILES: N#Cc1cc(-c2ccc(-n3c4ccccc4c4ccc5c6ccccc6oc5c43)c(C#N)c2)ccc1-n1c2ccccc2c2ccccc21 ; N#Cc1cc(-c2ccc(-n3c4ccccc4c4ccc5oc6ccccc6c5c43)c(C#N)c2)ccc1-n1c2ccccc2c2ccccc21 ; N#Cc1cc(-c2ccc(-n3c4ccccc4c4cc5c(cc43)oc3ccccc35)c(C#N)c2)ccc1-n1c2ccccc2c2ccccc21 ; N#Cc1cc(-c2ccc(-n3c4ccccc4c4cc5oc6ccccc6c5cc43)c(C#N)c2)ccc1-n1c2ccccc2c2ccccc21 ; N#Cc1cc(-c2ccc(-n3c4ccccc4c4c5c(ccc43)oc3ccccc35)c(C#N)c2)ccc1-n1c2ccccc2c2ccccc21 ; N#Cc1cc(-c2ccc(-n3c4ccccc4c4c5oc6ccccc6c5ccc43)c(C#N)c2)ccc1-n1c2ccccc2c2ccccc21]

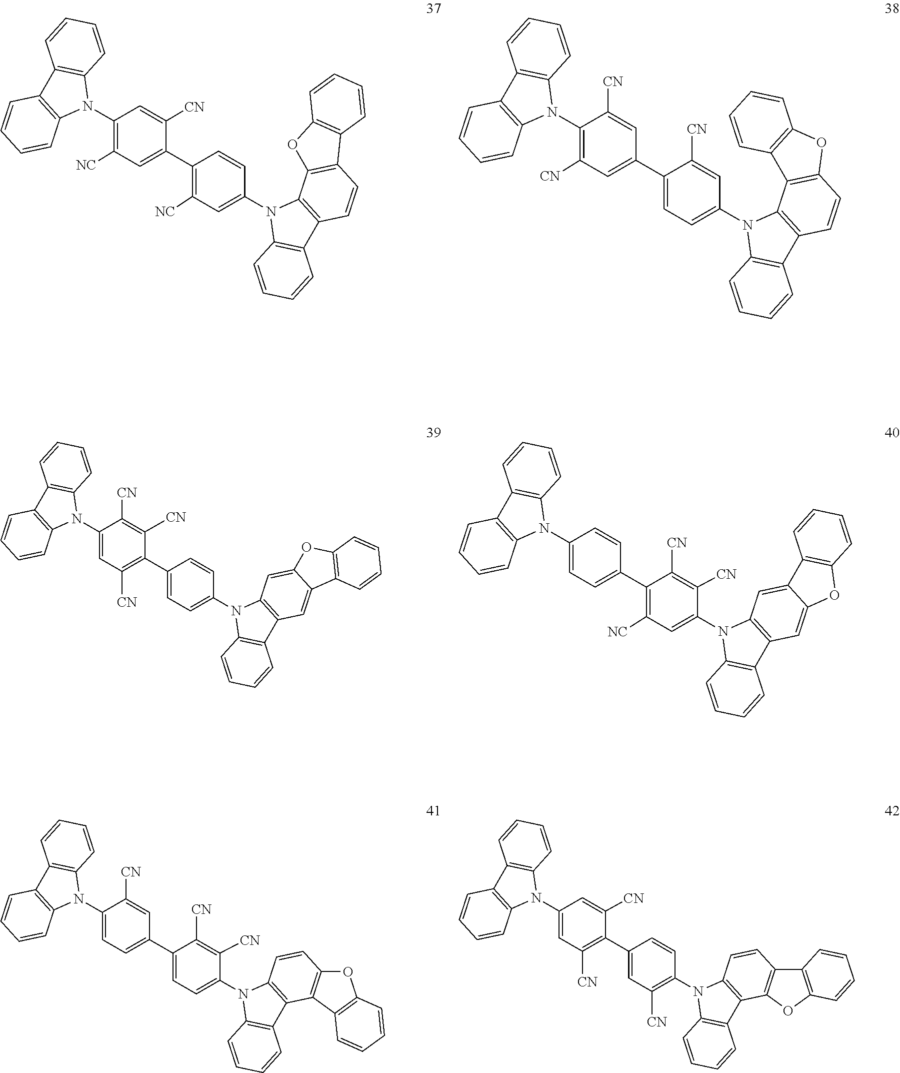

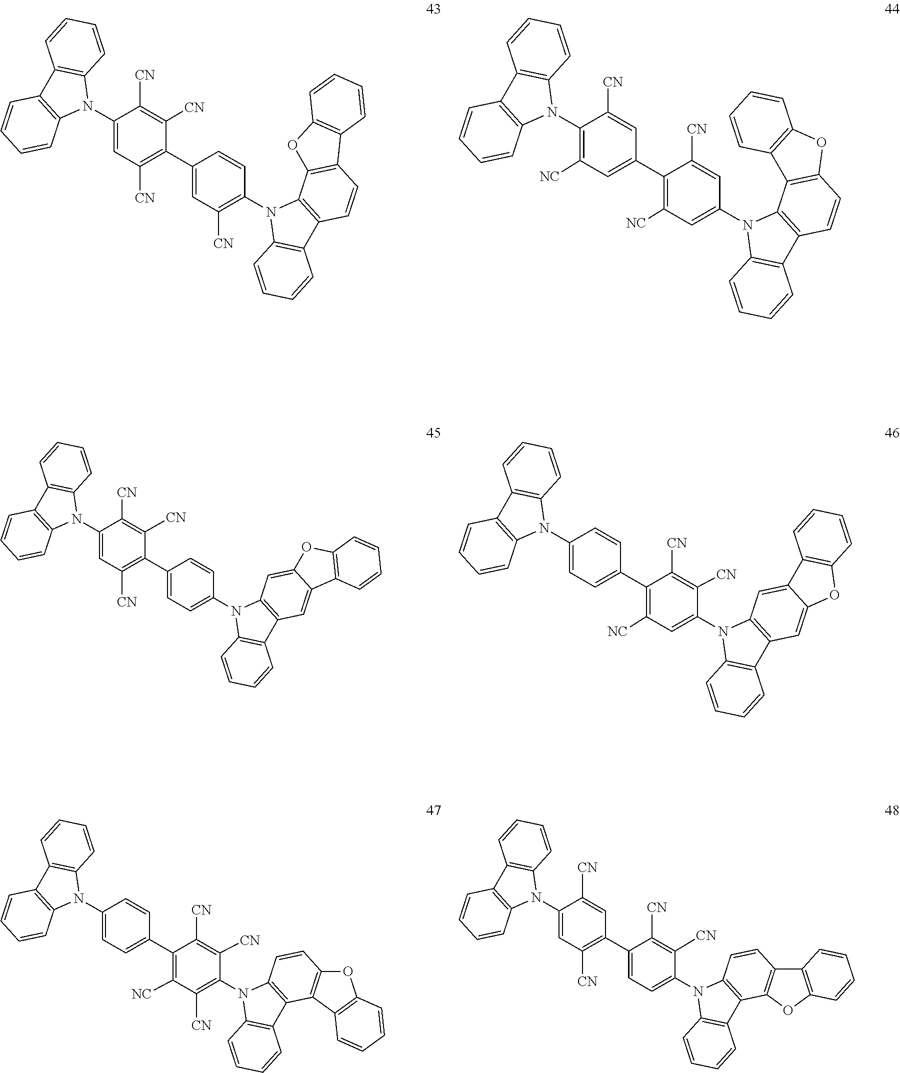

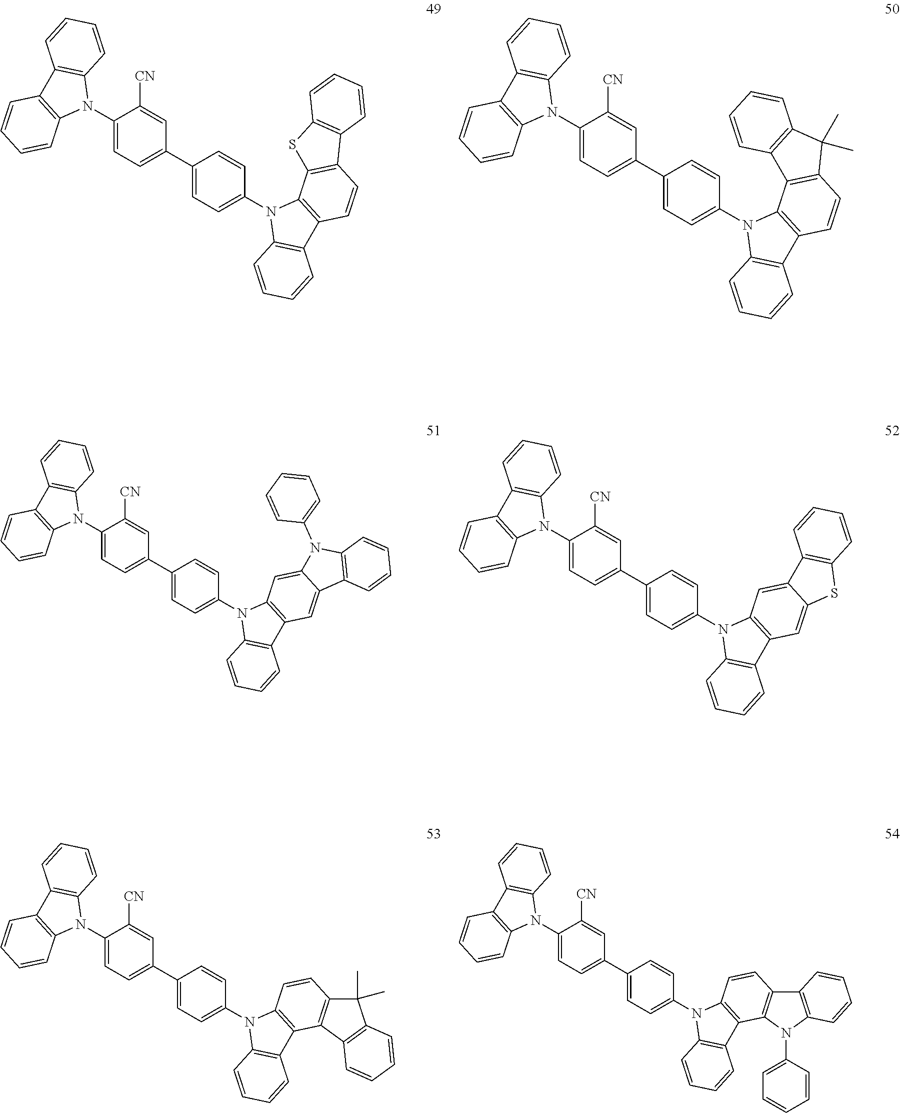

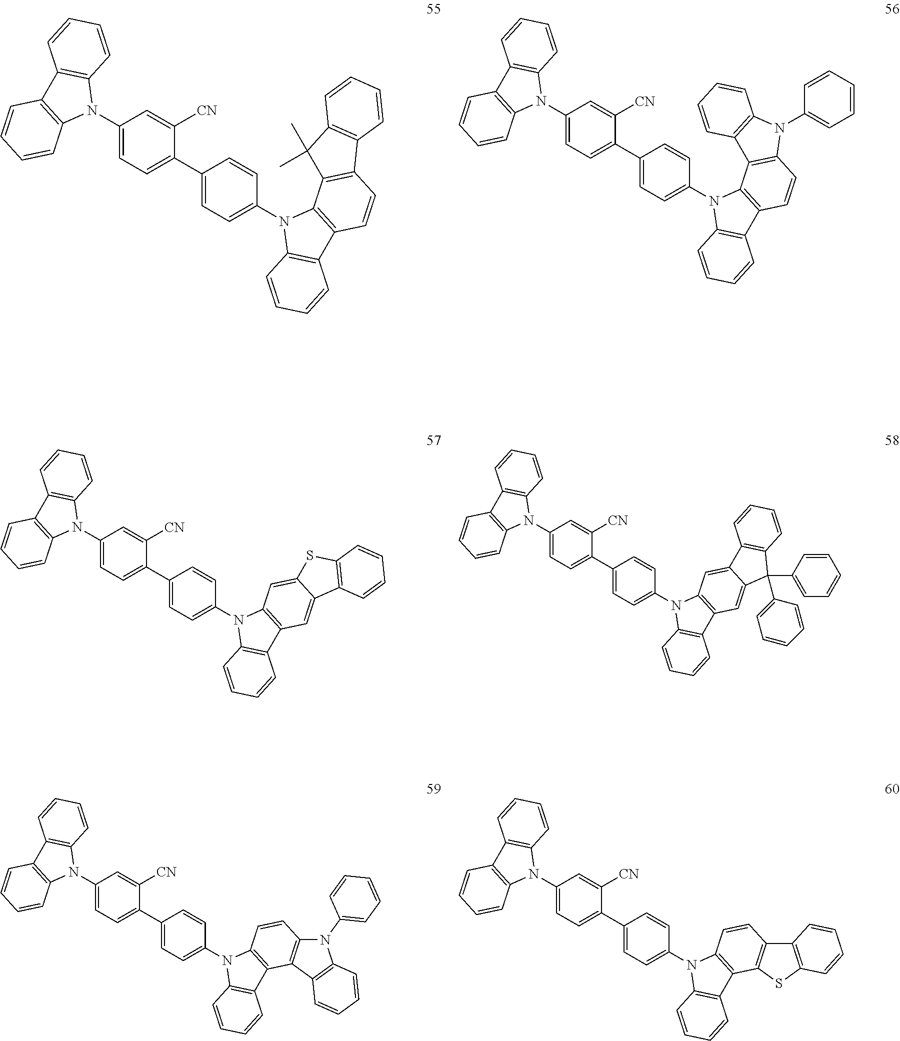

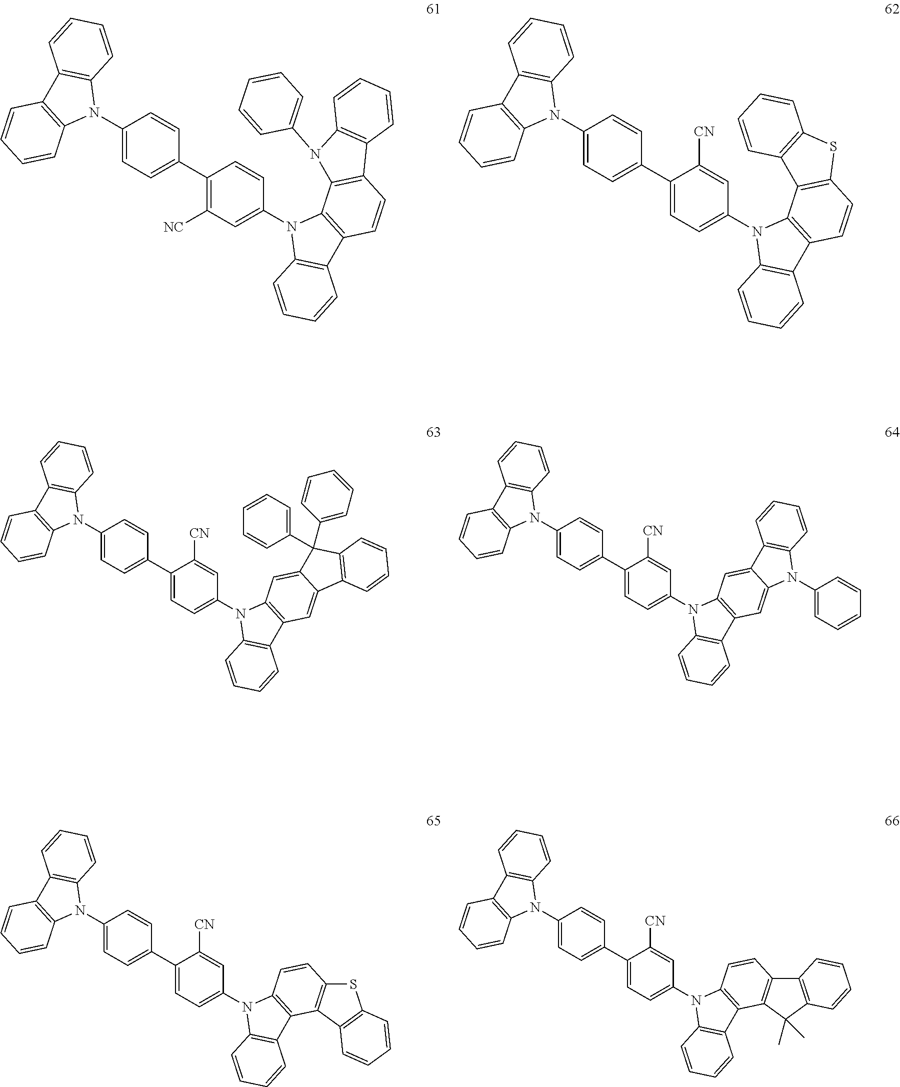

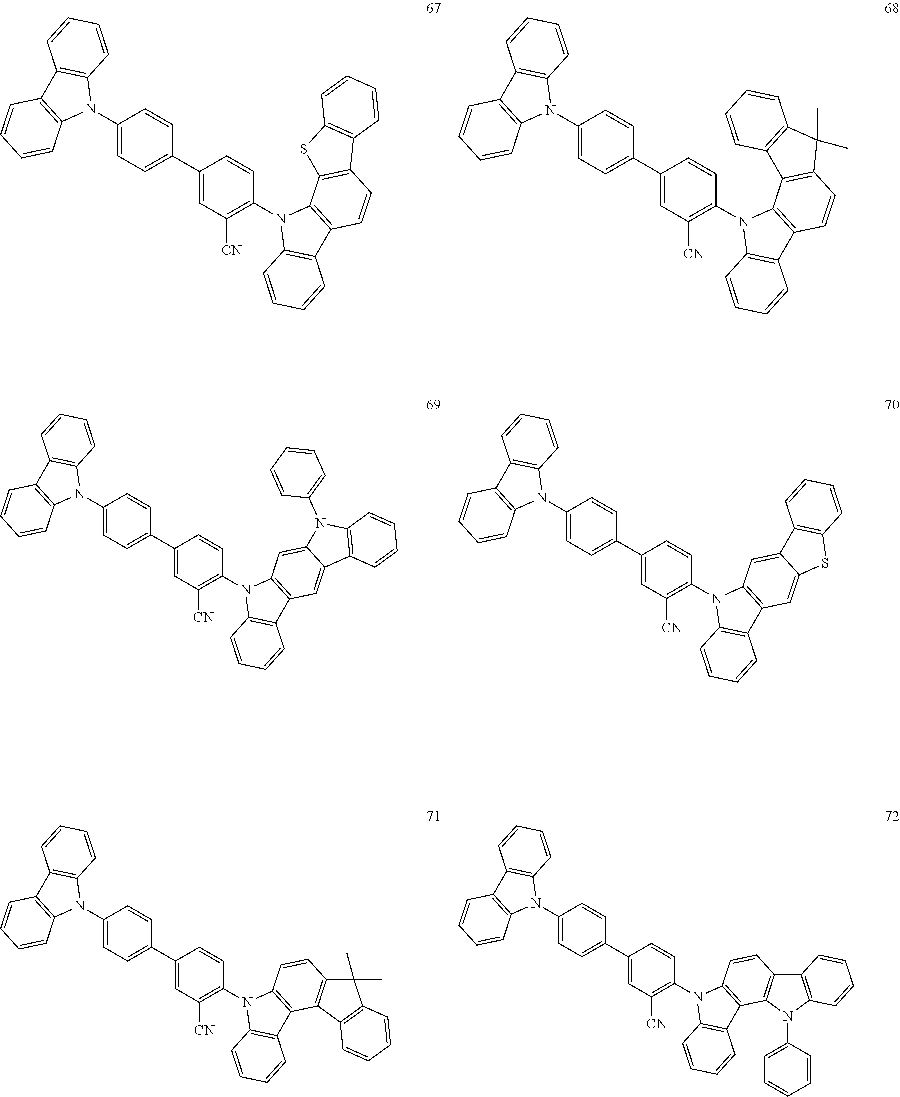 [SMILES: N#Cc1cc(-c2ccc(-n3c4ccccc4c4ccccc43)cc2)ccc1-n1c2ccccc2c2ccc3c4ccccc4sc3c21 ; CC1(C)c2ccccc2-c2c1ccc1c3ccccc3n(-c3ccc(-c4ccc(-n5c6ccccc6c6ccccc65)cc4)cc3C#N)c21 ; N#Cc1cc(-c2ccc(-n3c4ccccc4c4ccccc43)cc2)ccc1-n1c2ccccc2c2cc3c4ccccc4n(-c4ccccc4)c3cc21 ; N#Cc1cc(-c2ccc(-n3c4ccccc4c4ccccc43)cc2)ccc1-n1c2ccccc2c2cc3sc4ccccc4c3cc21 ; CC1(C)c2ccccc2-c2c1ccc1c2c2ccccc2n1-c1ccc(-c2ccc(-n3c4ccccc4c4ccccc43)cc2)cc1C#N ; N#Cc1cc(-c2ccc(-n3c4ccccc4c4ccccc43)cc2)ccc1-n1c2ccccc2c2c1ccc1c3ccccc3n(-c3ccccc3)c12]

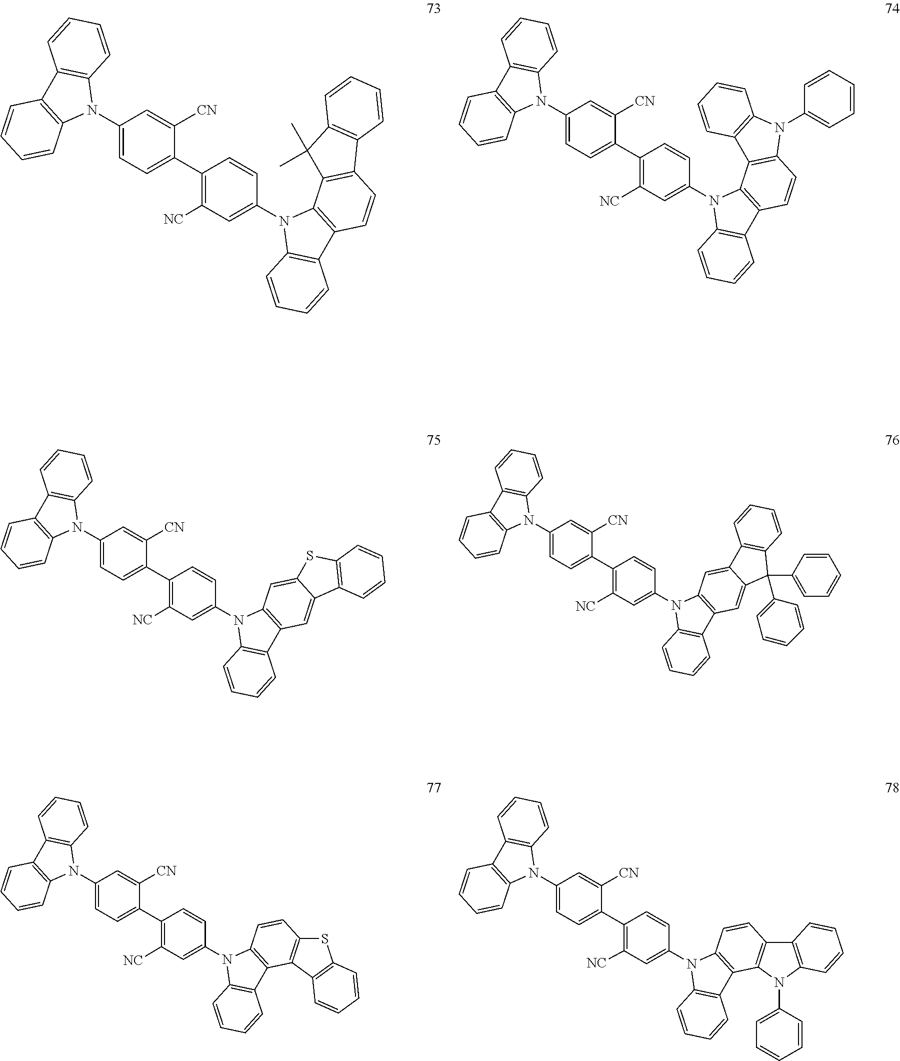

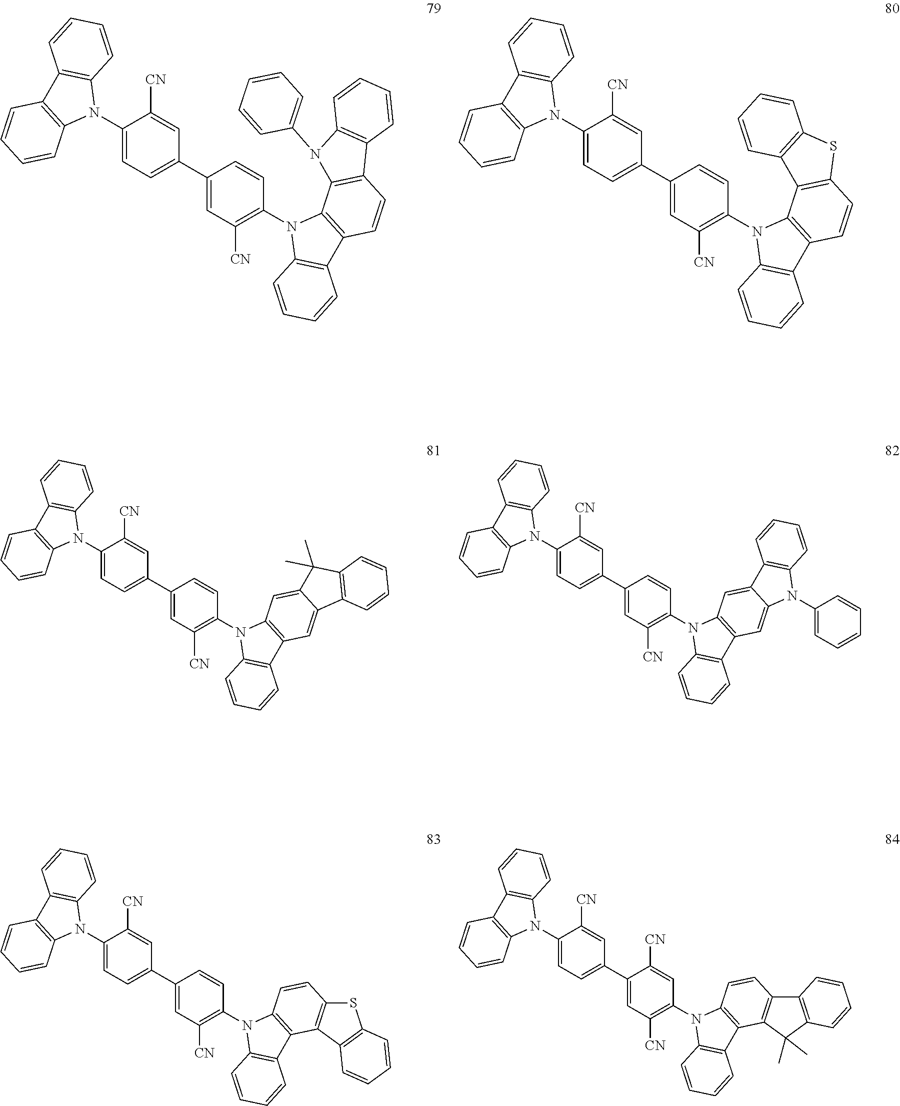

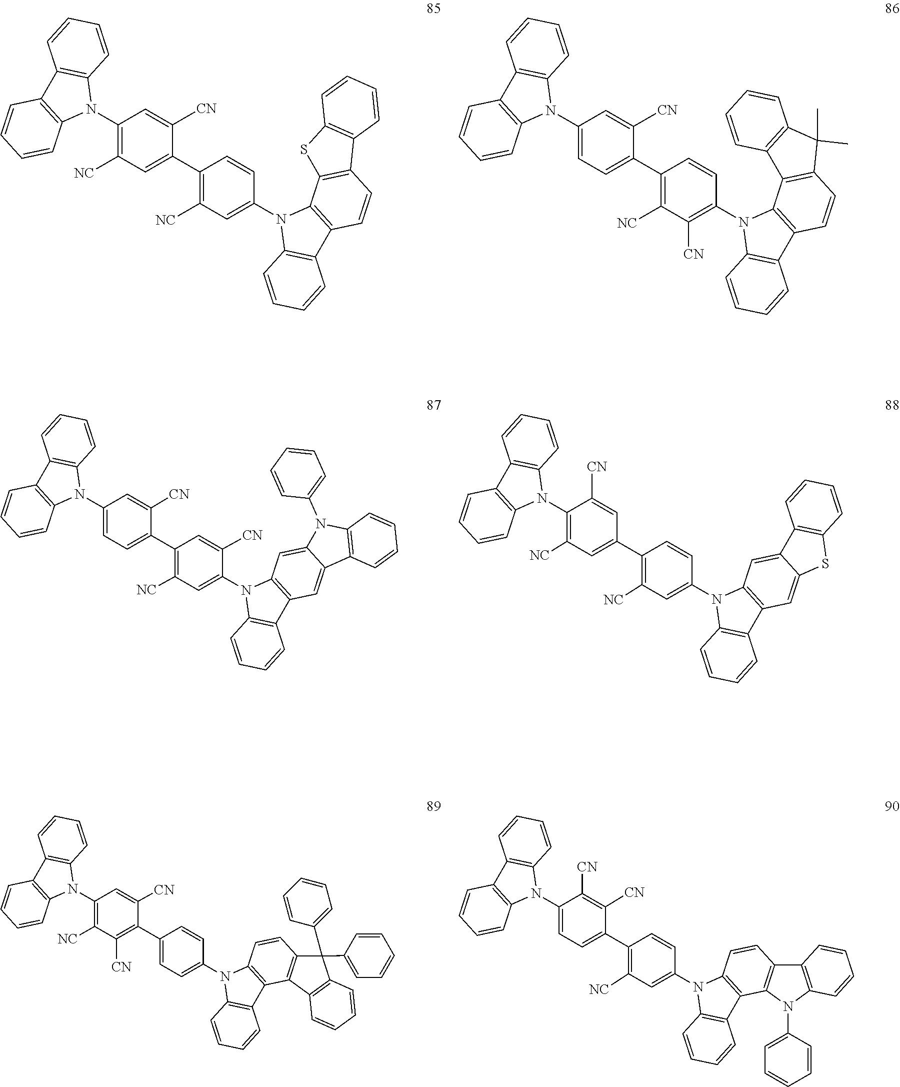

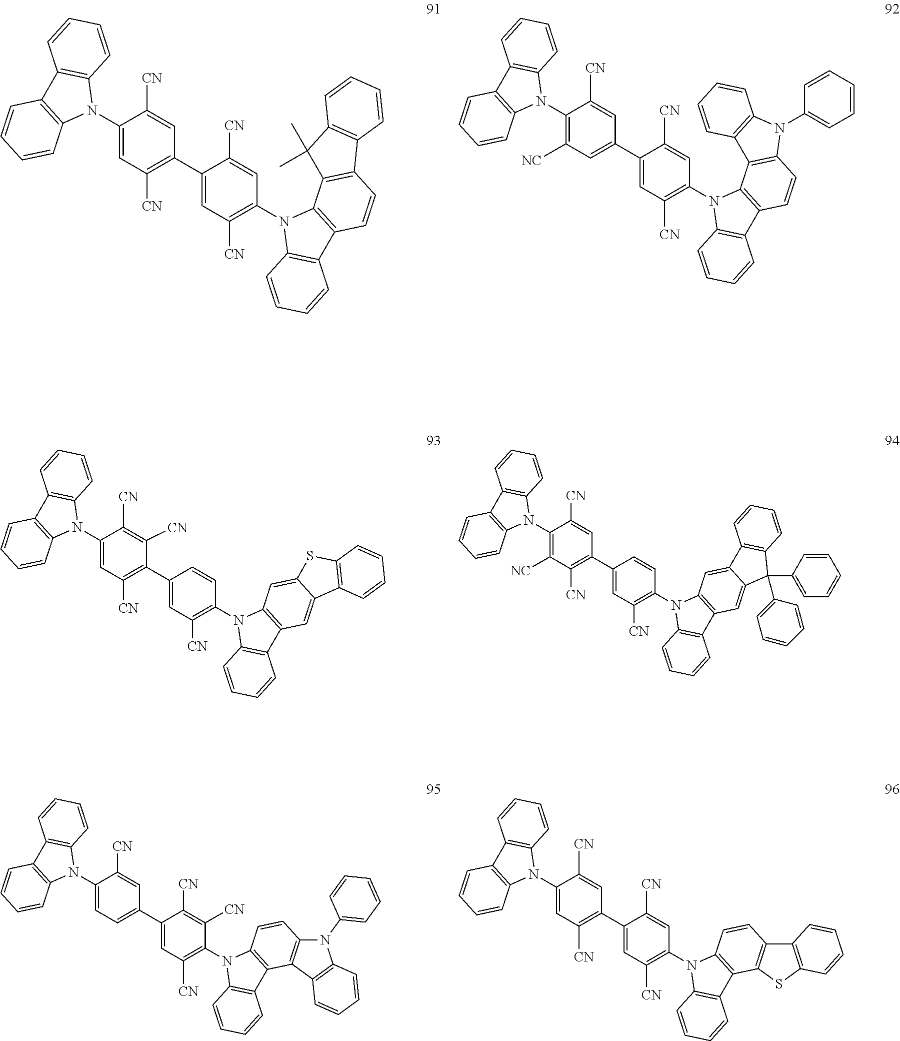

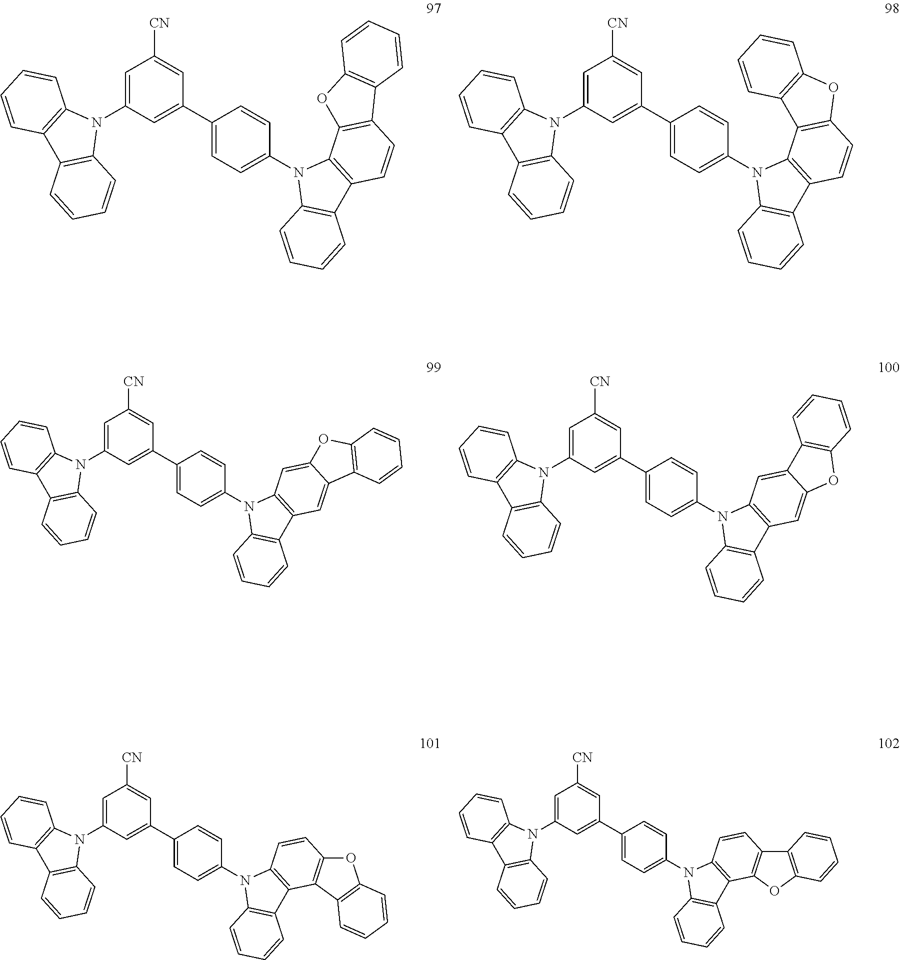 [SMILES: N#Cc1cc(-c2ccc(-n3c4ccccc4c4ccc5c6ccccc6oc5c43)cc2)cc(-n2c3ccccc3c3ccccc32)c1 ; N#Cc1cc(-c2ccc(-n3c4ccccc4c4ccc5oc6ccccc6c5c43)cc2)cc(-n2c3ccccc3c3ccccc32)c1 ; N#Cc1cc(-c2ccc(-n3c4ccccc4c4cc5c(cc43)oc3ccccc35)cc2)cc(-n2c3ccccc3c3ccccc32)c1 ; N#Cc1cc(-c2ccc(-n3c4ccccc4c4cc5oc6ccccc6c5cc43)cc2)cc(-n2c3ccccc3c3ccccc32)c1 ; N#Cc1cc(-c2ccc(-n3c4ccccc4c4c5c(ccc43)oc3ccccc35)cc2)cc(-n2c3ccccc3c3ccccc32)c1 ; N#Cc1cc(-c2ccc(-n3c4ccccc4c4c5oc6ccccc6c5ccc43)cc2)cc(-n2c3ccccc3c3ccccc32)c1]

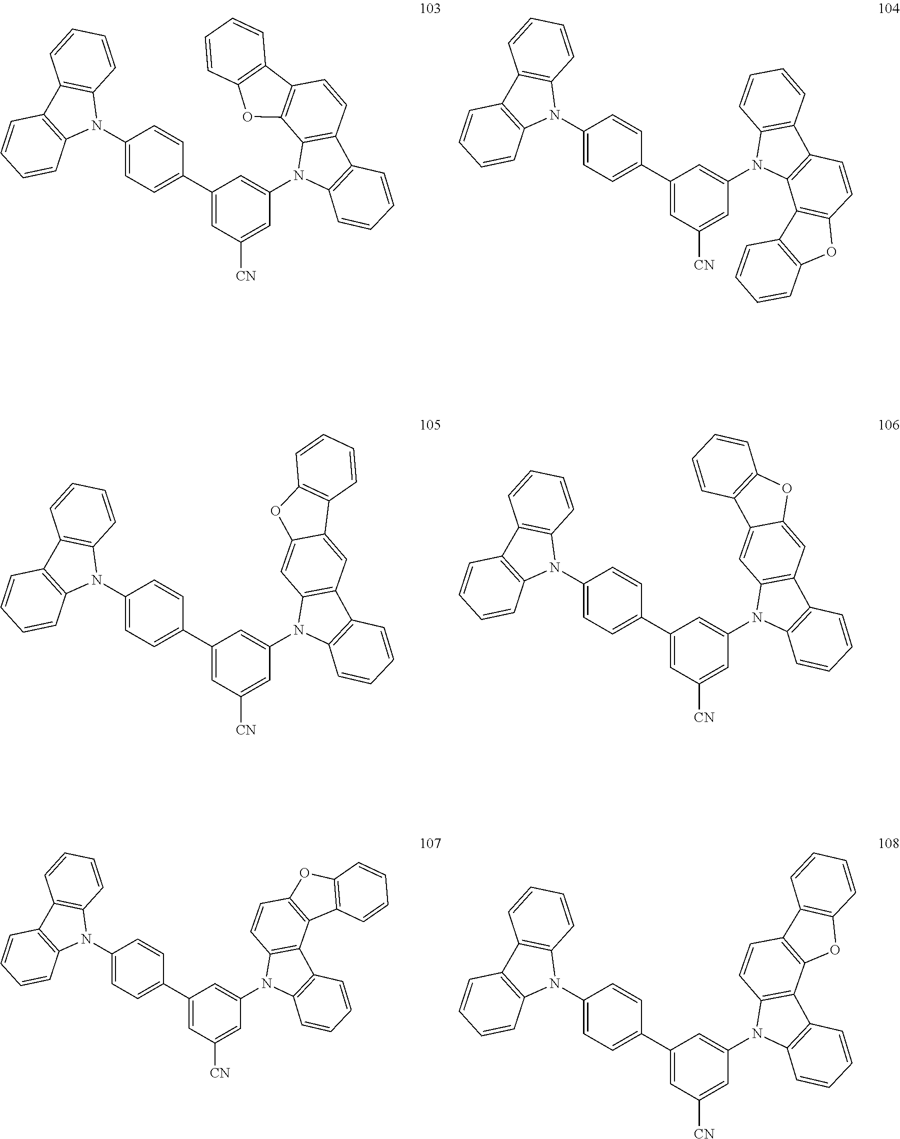 [SMILES: N#Cc1cc(-c2ccc(-n3c4ccccc4c4ccccc43)cc2)cc(-n2c3ccccc3c3ccc4c5ccccc5oc4c32)c1 ; N#Cc1cc(-c2ccc(-n3c4ccccc4c4ccccc43)cc2)cc(-n2c3ccccc3c3ccc4oc5ccccc5c4c32)c1 ; N#Cc1cc(-c2ccc(-n3c4ccccc4c4ccccc43)cc2)cc(-n2c3ccccc3c3cc4c(cc32)oc2ccccc24)c1 ; N#Cc1cc(-c2ccc(-n3c4ccccc4c4ccccc43)cc2)cc(-n2c3ccccc3c3cc4oc5ccccc5c4cc32)c1 ; N#Cc1cc(-c2ccc(-n3c4ccccc4c4ccccc43)cc2)cc(-n2c3ccccc3c3c4c(ccc32)oc2ccccc24)c1 ; N#Cc1cc(-c2ccc(-n3c4ccccc4c4ccccc43)cc2)cc(-n2c3ccccc3c3c4oc5ccccc5c4ccc32)c1]

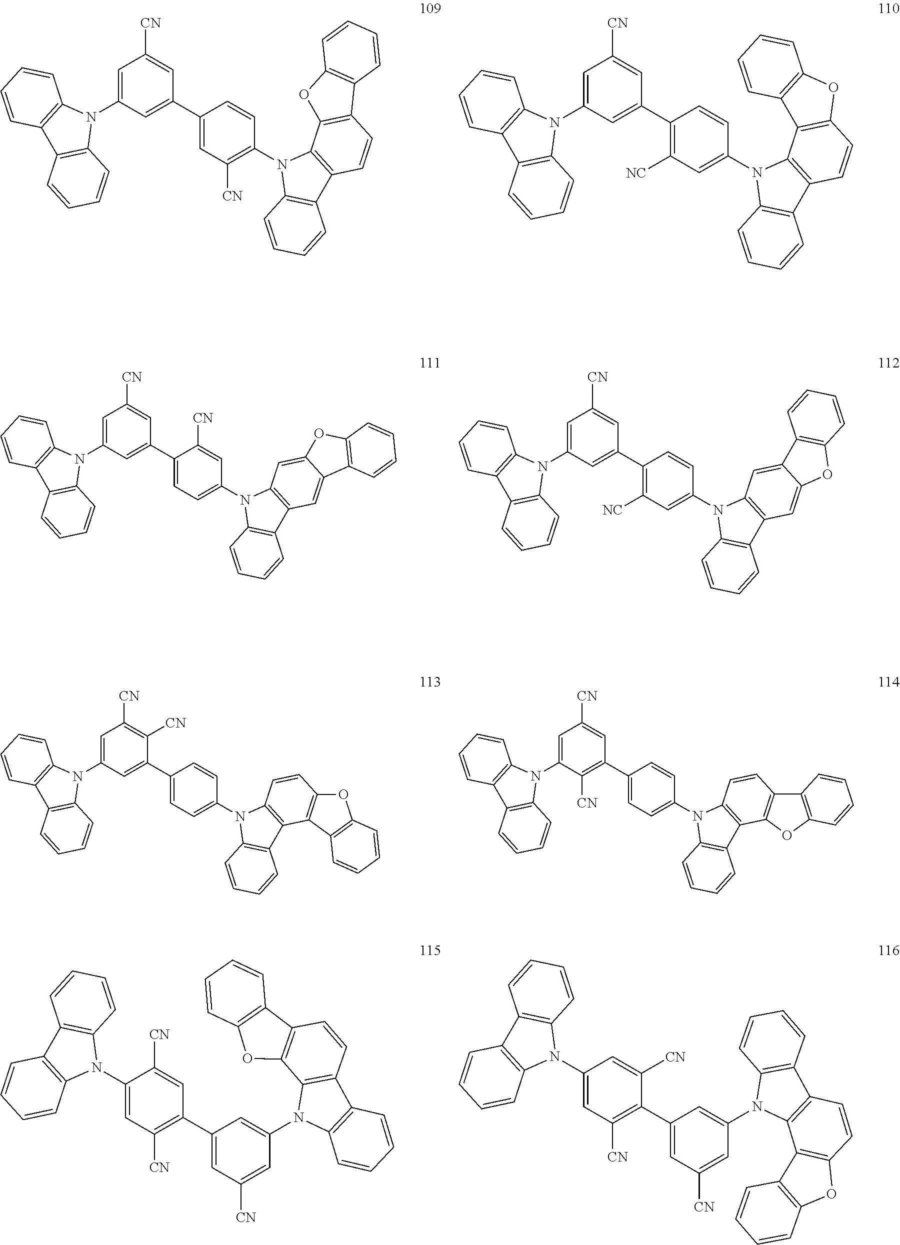

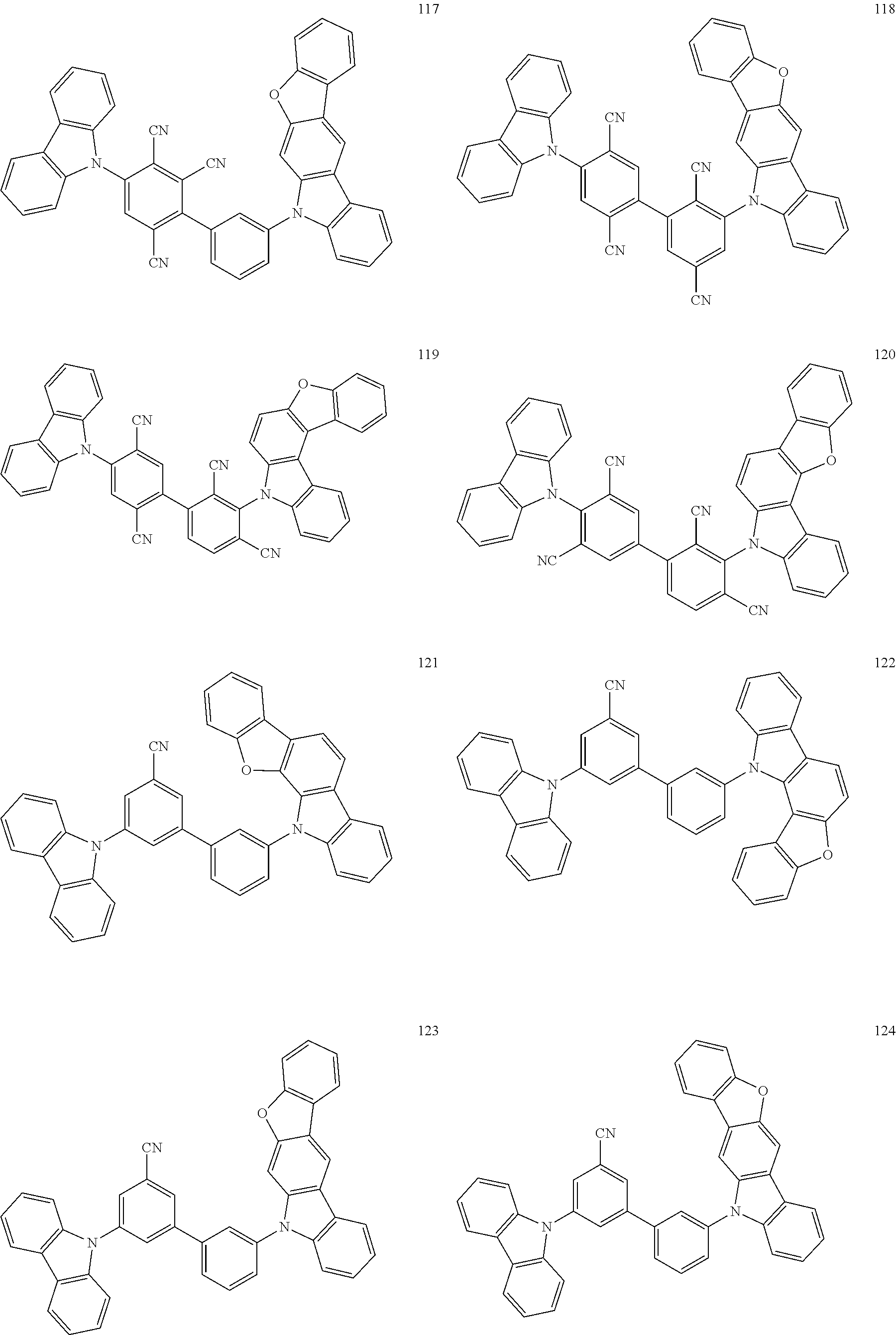

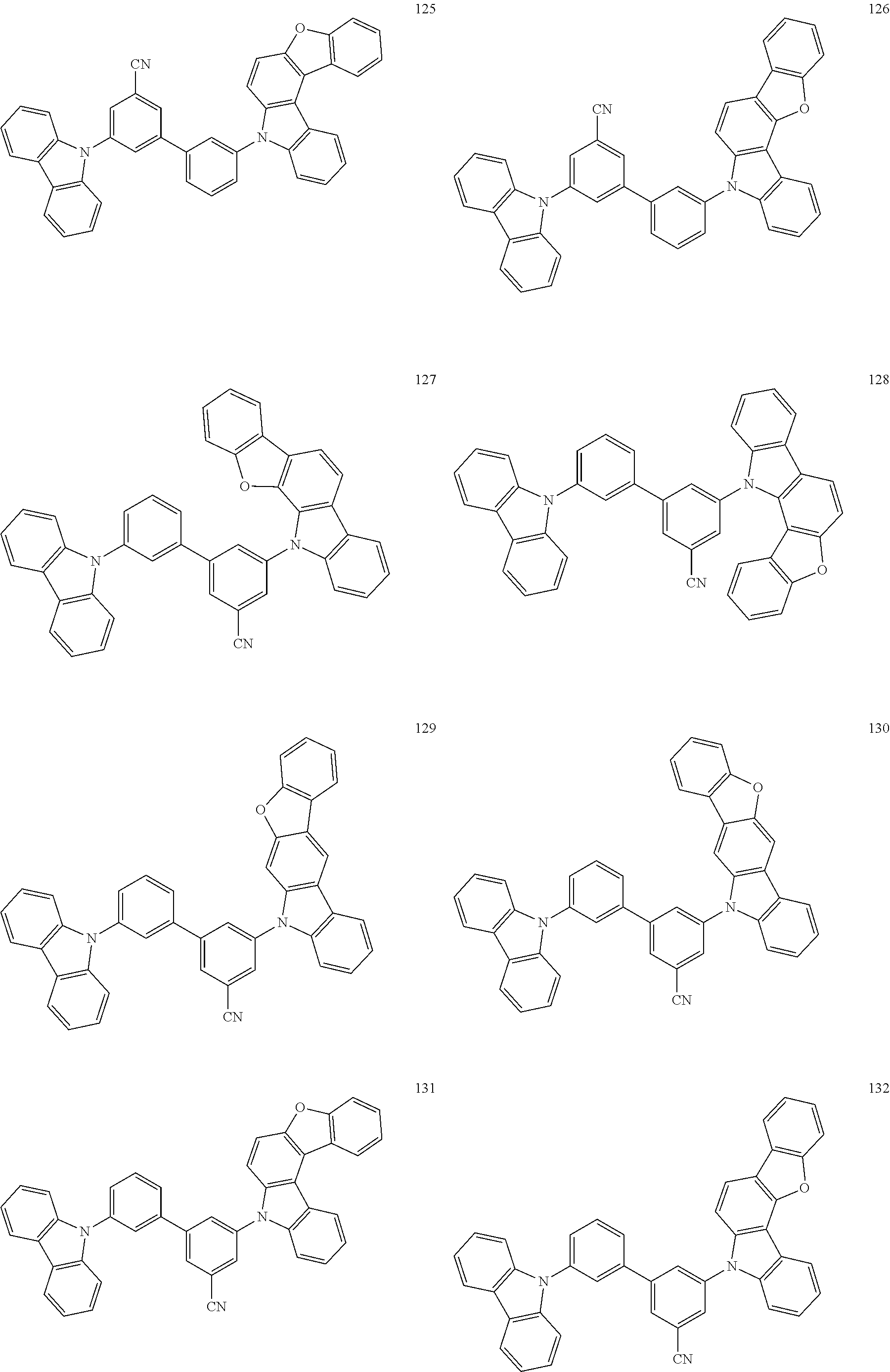

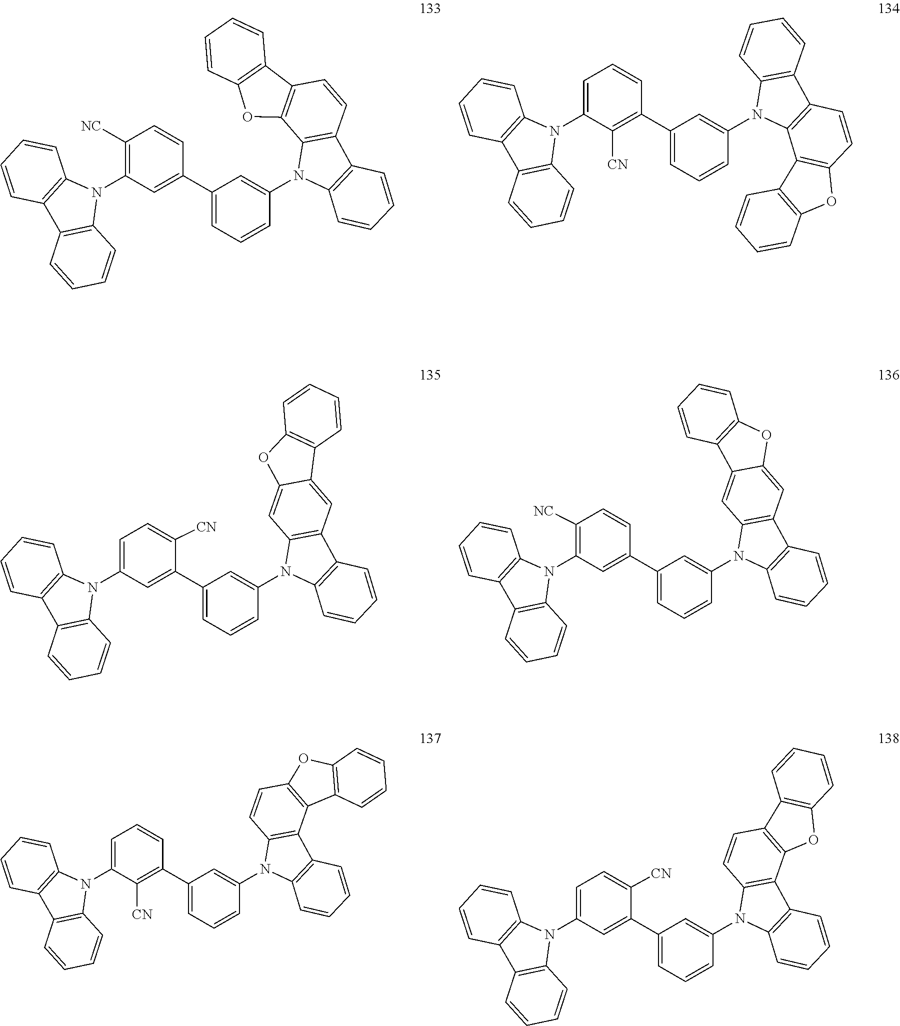 [SMILES: N#Cc1ccc(-c2cccc(-n3c4ccccc4c4ccc5c6ccccc6oc5c43)c2)cc1-n1c2ccccc2c2ccccc21 ; N#Cc1c(-c2cccc(-n3c4ccccc4c4ccc5oc6ccccc6c5c43)c2)cccc1-n1c2ccccc2c2ccccc21 ; N#Cc1ccc(-n2c3ccccc3c3ccccc32)cc1-c1cccc(-n2c3ccccc3c3cc4c(cc32)oc2ccccc24)c1 ; N#Cc1ccc(-c2cccc(-n3c4ccccc4c4cc5oc6ccccc6c5cc43)c2)cc1-n1c2ccccc2c2ccccc21 ; N#Cc1c(-c2cccc(-n3c4ccccc4c4c5c(ccc43)oc3ccccc35)c2)cccc1-n1c2ccccc2c2ccccc21 ; N#Cc1ccc(-n2c3ccccc3c3ccccc32)cc1-c1cccc(-n2c3ccccc3c3c4oc5ccccc5c4ccc32)c1]

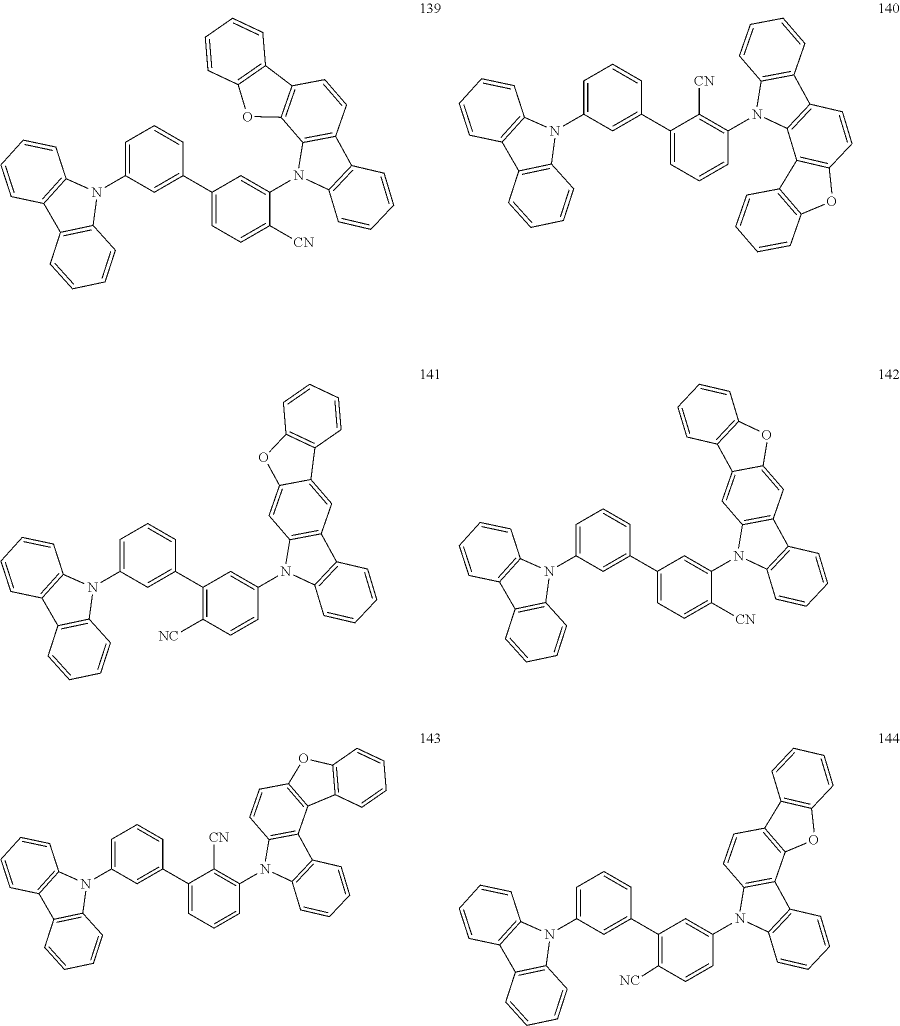 [SMILES: N#Cc1ccc(-c2cccc(-n3c4ccccc4c4ccccc43)c2)cc1-n1c2ccccc2c2ccc3c4ccccc4oc3c21 ; N#Cc1c(-c2cccc(-n3c4ccccc4c4ccccc43)c2)cccc1-n1c2ccccc2c2ccc3oc4ccccc4c3c21 ; N#Cc1ccc(-n2c3ccccc3c3cc4c(cc32)oc2ccccc24)cc1-c1cccc(-n2c3ccccc3c3ccccc32)c1 ; N#Cc1ccc(-c2cccc(-n3c4ccccc4c4ccccc43)c2)cc1-n1c2ccccc2c2cc3oc4ccccc4c3cc21 ; N#Cc1c(-c2cccc(-n3c4ccccc4c4ccccc43)c2)cccc1-n1c2ccccc2c2c3c(ccc21)oc1ccccc13 ; N#Cc1ccc(-n2c3ccccc3c3c4oc5ccccc5c4ccc32)cc1-c1cccc(-n2c3ccccc3c3ccccc32)c1]

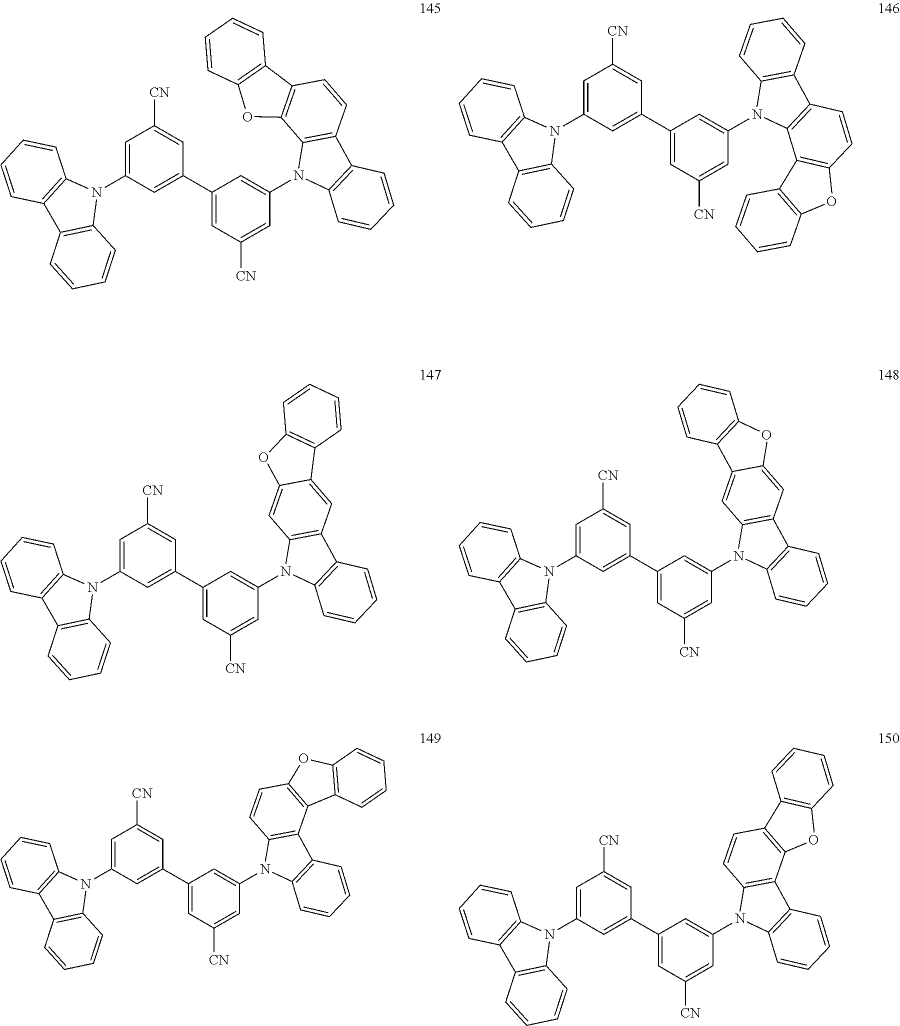

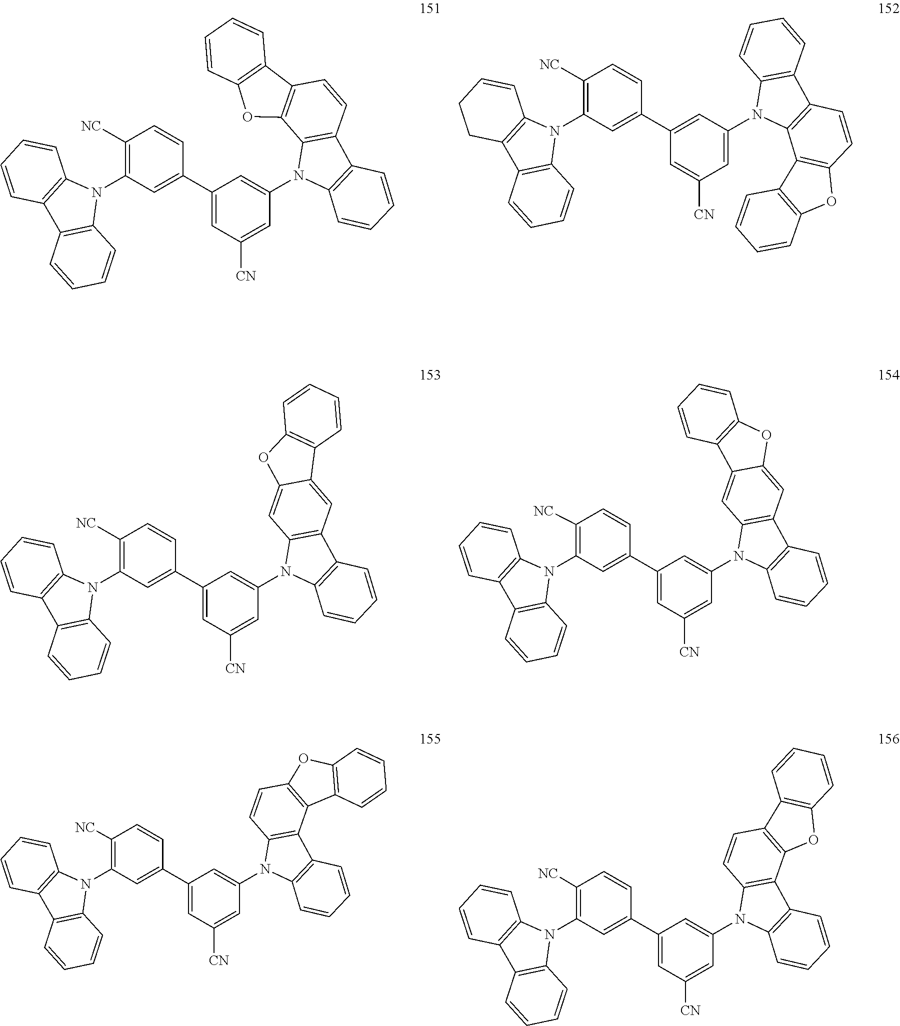

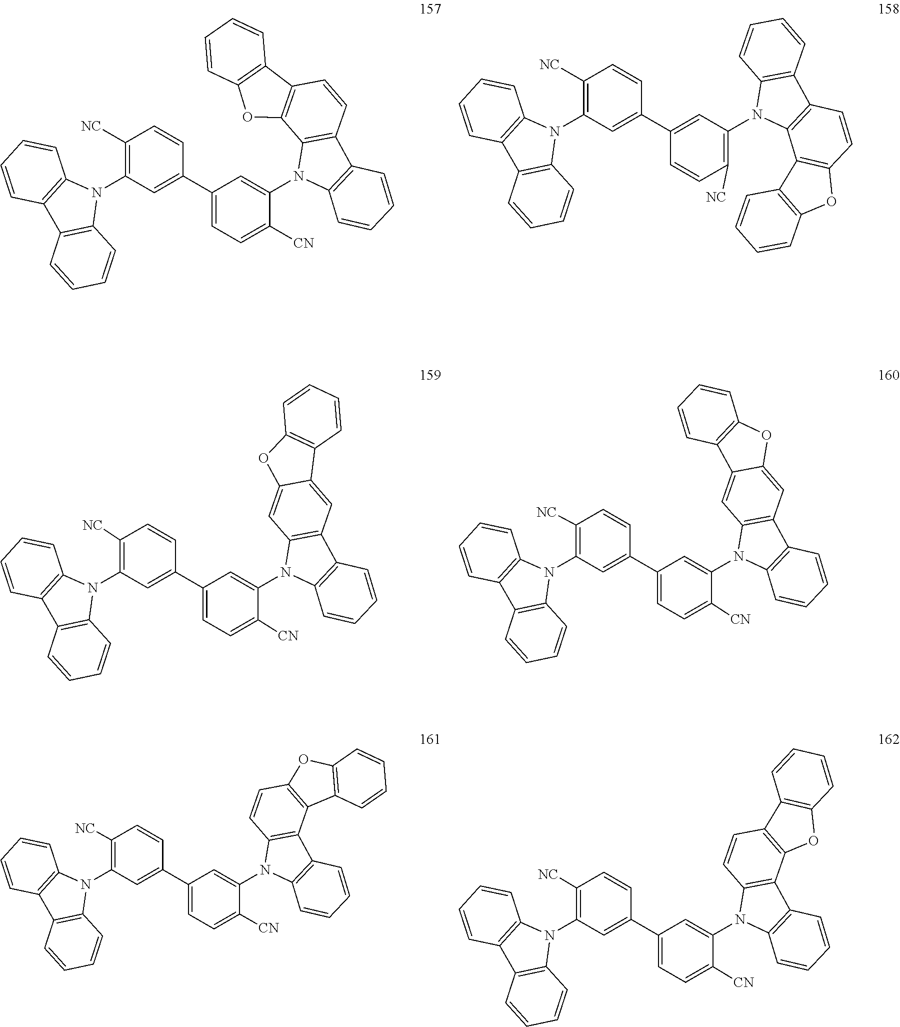 [SMILES: N#Cc1ccc(-c2ccc(C#N)c(-n3c4ccccc4c4ccc5c6ccccc6oc5c43)c2)cc1-n1c2ccccc2c2ccccc21 ; N#Cc1ccc(-c2ccc(C#N)c(-n3c4ccccc4c4ccc5oc6ccccc6c5c43)c2)cc1-n1c2ccccc2c2ccccc21 ; N#Cc1ccc(-c2ccc(C#N)c(-n3c4ccccc4c4cc5c(cc43)oc3ccccc35)c2)cc1-n1c2ccccc2c2ccccc21 ; N#Cc1ccc(-c2ccc(C#N)c(-n3c4ccccc4c4cc5oc6ccccc6c5cc43)c2)cc1-n1c2ccccc2c2ccccc21 ; N#Cc1ccc(-c2ccc(C#N)c(-n3c4ccccc4c4c5c(ccc43)oc3ccccc35)c2)cc1-n1c2ccccc2c2ccccc21 ; N#Cc1ccc(-c2ccc(C#N)c(-n3c4ccccc4c4c5oc6ccccc6c5ccc43)c2)cc1-n1c2ccccc2c2ccccc21]

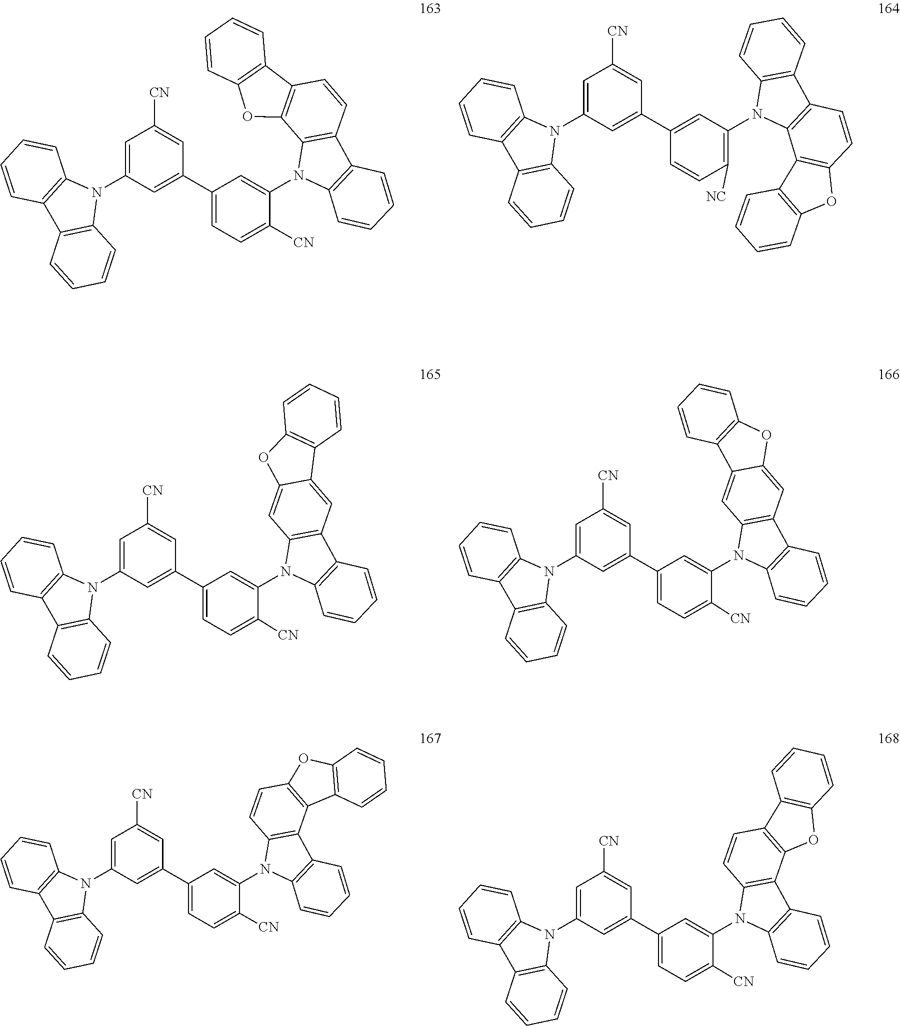

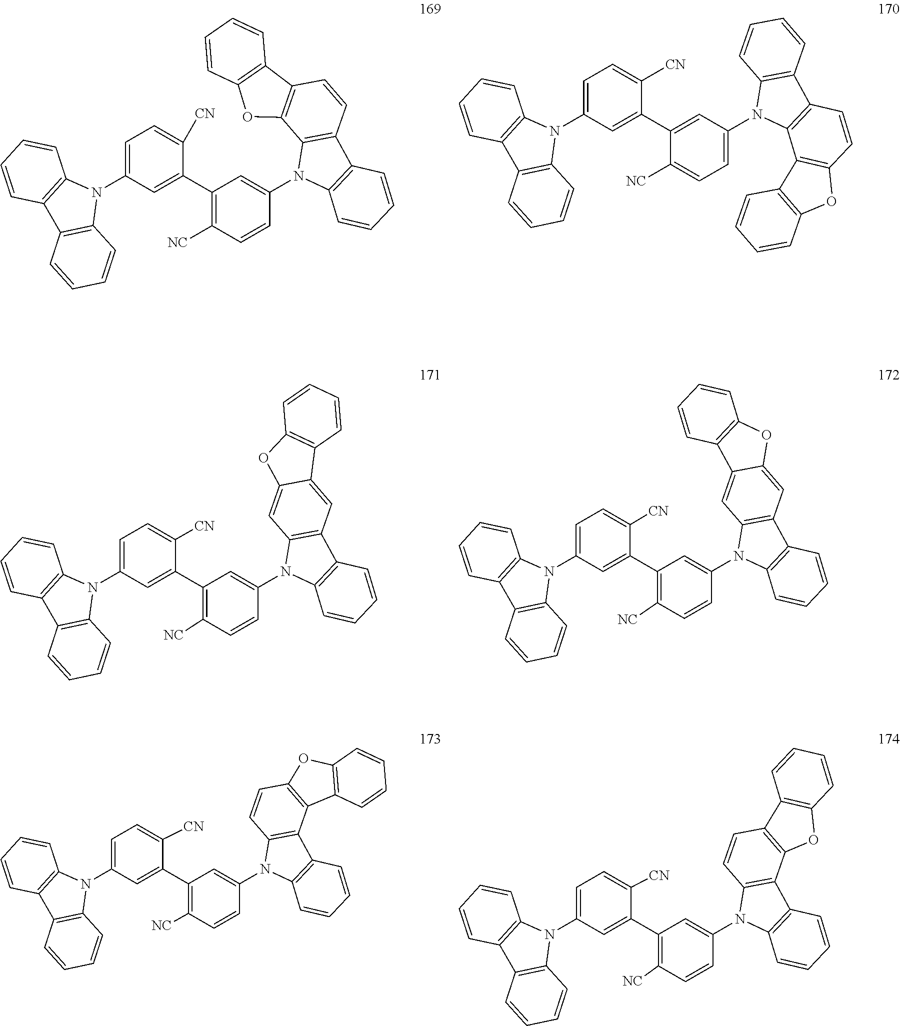 [SMILES: N#Cc1ccc(-n2c3ccccc3c3ccccc32)cc1-c1cc(-n2c3ccccc3c3ccc4c5ccccc5oc4c32)ccc1C#N ; N#Cc1ccc(-n2c3ccccc3c3ccccc32)cc1-c1cc(-n2c3ccccc3c3ccc4oc5ccccc5c4c32)ccc1C#N ; N#Cc1ccc(-n2c3ccccc3c3ccccc32)cc1-c1cc(-n2c3ccccc3c3cc4c(cc32)oc2ccccc24)ccc1C#N ; N#Cc1ccc(-n2c3ccccc3c3ccccc32)cc1-c1cc(-n2c3ccccc3c3cc4oc5ccccc5c4cc32)ccc1C#N ; N#Cc1ccc(-n2c3ccccc3c3ccccc32)cc1-c1cc(-n2c3ccccc3c3c4c(ccc32)oc2ccccc24)ccc1C#N ; N#Cc1ccc(-n2c3ccccc3c3ccccc32)cc1-c1cc(-n2c3ccccc3c3c4oc5ccccc5c4ccc32)ccc1C#N]

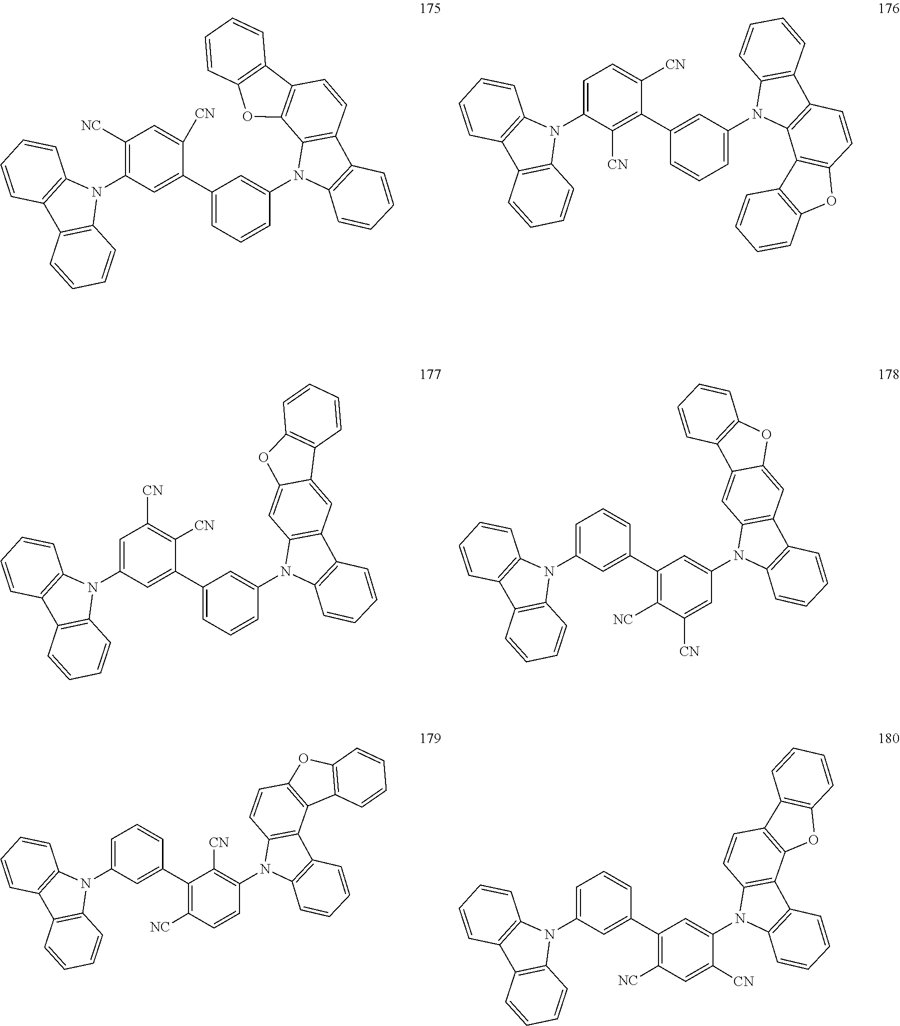 [SMILES: N#Cc1cc(C#N)c(-n2c3ccccc3c3ccccc32)cc1-c1cccc(-n2c3ccccc3c3ccc4c5ccccc5oc4c32)c1 ; N#Cc1ccc(-n2c3ccccc3c3ccccc32)c(C#N)c1-c1cccc(-n2c3ccccc3c3ccc4oc5ccccc5c4c32)c1 ; N#Cc1cc(-n2c3ccccc3c3ccccc32)cc(-c2cccc(-n3c4ccccc4c4cc5c(cc43)oc3ccccc35)c2)c1C#N ; N#Cc1cc(-n2c3ccccc3c3cc4oc5ccccc5c4cc32)cc(-c2cccc(-n3c4ccccc4c4ccccc43)c2)c1C#N ; N#Cc1ccc(-n2c3ccccc3c3c4c(ccc32)oc2ccccc24)c(C#N)c1-c1cccc(-n2c3ccccc3c3ccccc32)c1 ; N#Cc1cc(C#N)c(-n2c3ccccc3c3c4oc5ccccc5c4ccc32)cc1-c1cccc(-n2c3ccccc3c3ccccc32)c1]

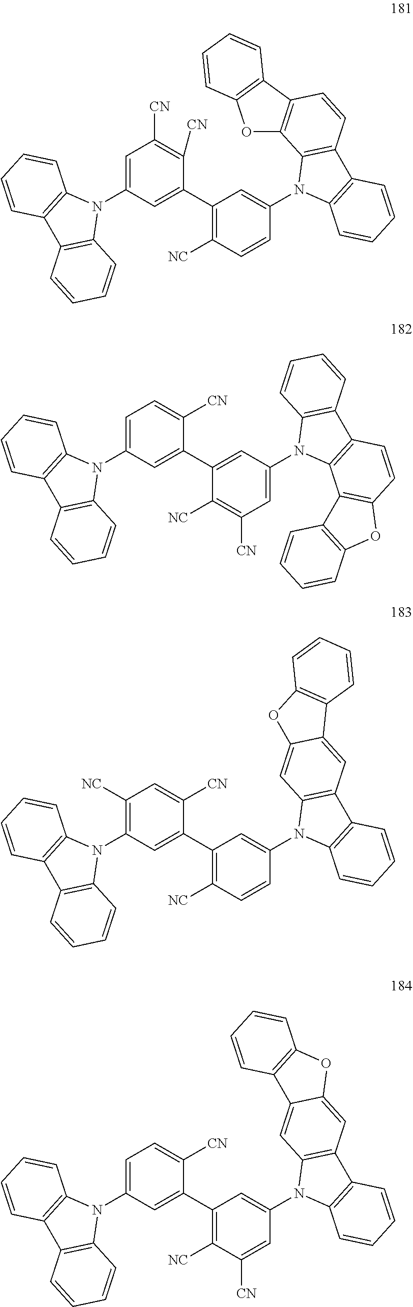 [SMILES: N#Cc1ccc(-n2c3ccccc3c3ccc4c5ccccc5oc4c32)cc1-c1cc(-n2c3ccccc3c3ccccc32)cc(C#N)c1C#N ; N#Cc1ccc(-n2c3ccccc3c3ccccc32)cc1-c1cc(-n2c3ccccc3c3ccc4oc5ccccc5c4c32)cc(C#N)c1C#N ; N#Cc1ccc(-n2c3ccccc3c3cc4c(cc32)oc2ccccc24)cc1-c1cc(-n2c3ccccc3c3ccccc32)c(C#N)cc1C#N ; N#Cc1ccc(-n2c3ccccc3c3ccccc32)cc1-c1cc(-n2c3ccccc3c3cc4oc5ccccc5c4cc32)cc(C#N)c1C#N]

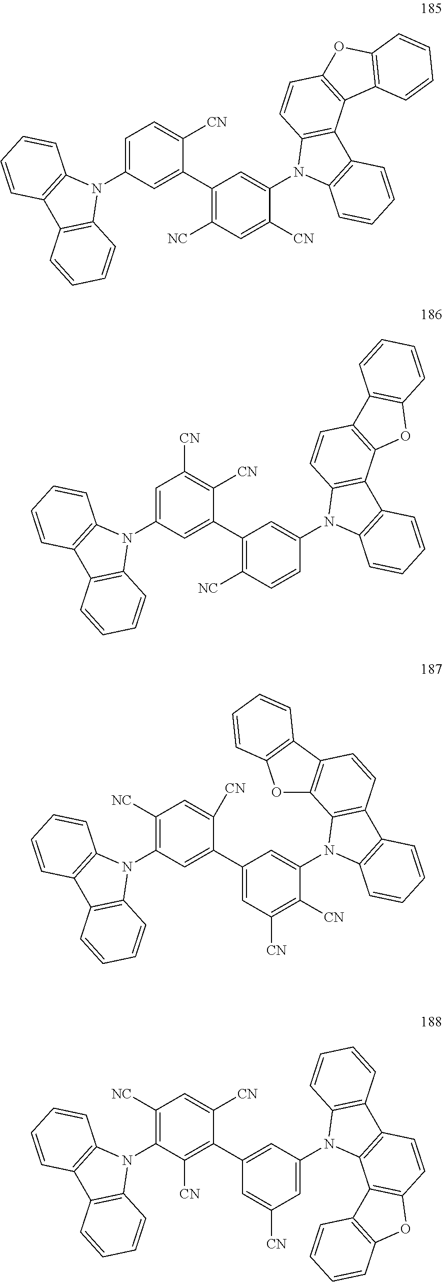 [SMILES: N#Cc1ccc(-n2c3ccccc3c3ccccc32)cc1-c1cc(-n2c3ccccc3c3c4c(ccc32)oc2ccccc24)c(C#N)cc1C#N ; N#Cc1ccc(-n2c3ccccc3c3c4oc5ccccc5c4ccc32)cc1-c1cc(-n2c3ccccc3c3ccccc32)cc(C#N)c1C#N ; N#Cc1cc(C#N)c(-n2c3ccccc3c3ccccc32)cc1-c1cc(C#N)c(C#N)c(-n2c3ccccc3c3ccc4c5ccccc5oc4c32)c1 ; N#Cc1cc(-c2c(C#N)cc(C#N)c(-n3c4ccccc4c4ccccc43)c2C#N)cc(-n2c3ccccc3c3ccc4oc5ccccc5c4c32)c1]

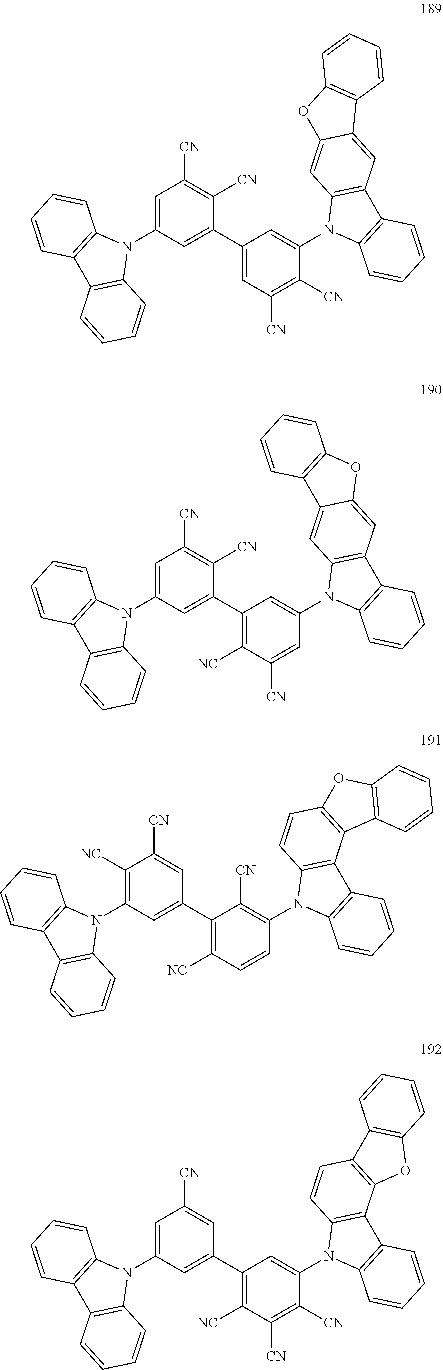 [SMILES: N#Cc1cc(-n2c3ccccc3c3ccccc32)cc(-c2cc(C#N)c(C#N)c(-n3c4ccccc4c4cc5c(cc43)oc3ccccc35)c2)c1C#N ; N#Cc1cc(-n2c3ccccc3c3ccccc32)cc(-c2cc(-n3c4ccccc4c4cc5oc6ccccc6c5cc43)cc(C#N)c2C#N)c1C#N ; N#Cc1cc(-c2c(C#N)ccc(-n3c4ccccc4c4c5c(ccc43)oc3ccccc35)c2C#N)cc(-n2c3ccccc3c3ccccc32)c1C#N ; N#Cc1cc(-c2cc(-n3c4ccccc4c4c5oc6ccccc6c5ccc43)c(C#N)c(C#N)c2C#N)cc(-n2c3ccccc3c3ccccc32)c1]

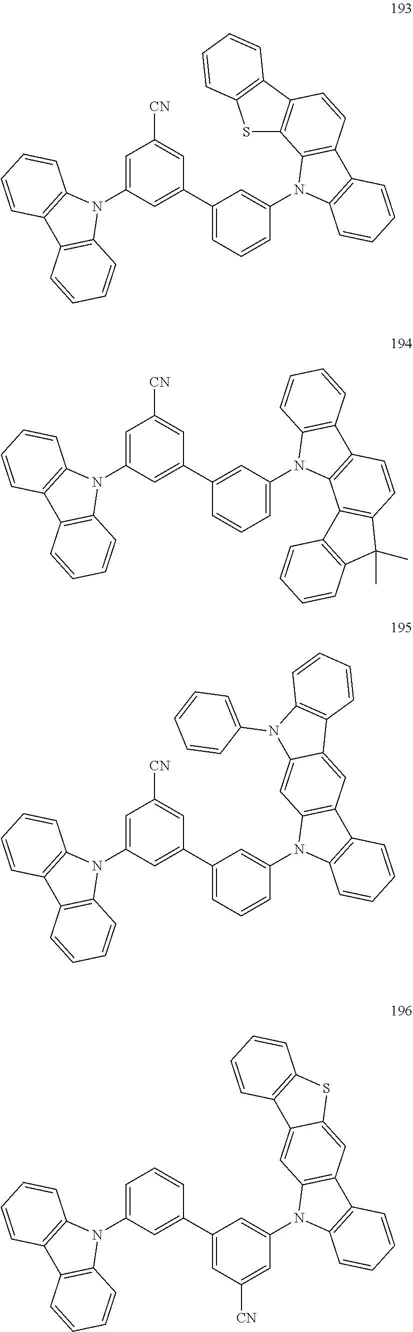 [SMILES: N#Cc1cc(-c2cccc(-n3c4ccccc4c4ccc5c6ccccc6sc5c43)c2)cc(-n2c3ccccc3c3ccccc32)c1 ; CC1(C)c2ccccc2-c2c1ccc1c3ccccc3n(-c3cccc(-c4cc(C#N)cc(-n5c6ccccc6c6ccccc65)c4)c3)c21 ; N#Cc1cc(-c2cccc(-n3c4ccccc4c4cc5c6ccccc6n(-c6ccccc6)c5cc43)c2)cc(-n2c3ccccc3c3ccccc32)c1 ; N#Cc1cc(-c2cccc(-n3c4ccccc4c4ccccc43)c2)cc(-n2c3ccccc3c3cc4sc5ccccc5c4cc32)c1]

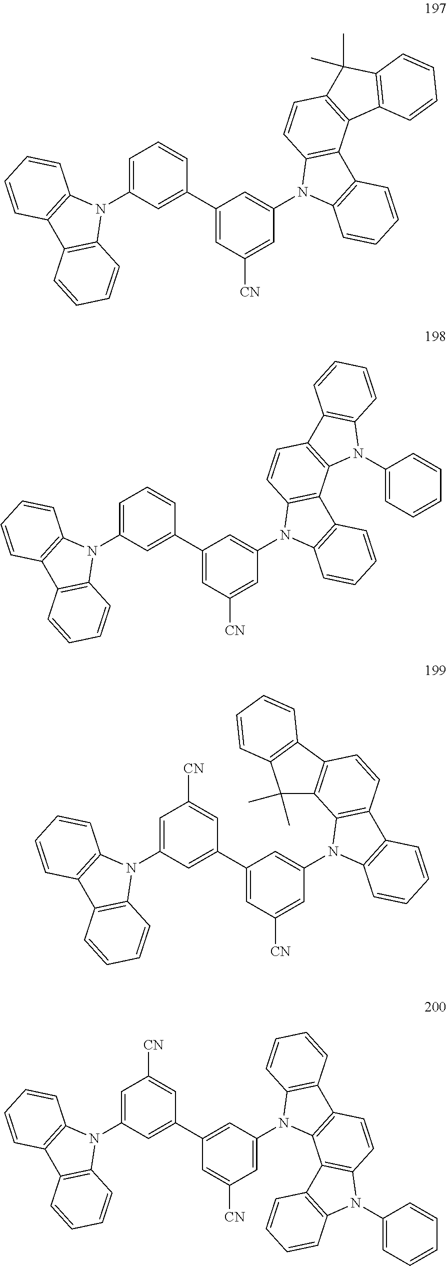 [SMILES: CC1(C)c2ccccc2-c2c1ccc1c2c2ccccc2n1-c1cc(C#N)cc(-c2cccc(-n3c4ccccc4c4ccccc43)c2)c1 ; N#Cc1cc(-c2cccc(-n3c4ccccc4c4ccccc43)c2)cc(-n2c3ccccc3c3c2ccc2c4ccccc4n(-c4ccccc4)c23)c1 ; CC1(C)c2ccccc2-c2ccc3c4ccccc4n(-c4cc(C#N)cc(-c5cc(C#N)cc(-n6c7ccccc7c7ccccc76)c5)c4)c3c21 ; N#Cc1cc(-c2cc(C#N)cc(-n3c4ccccc4c4ccc5c(c6ccccc6n5-c5ccccc5)c43)c2)cc(-n2c3ccccc3c3ccccc32)c1]

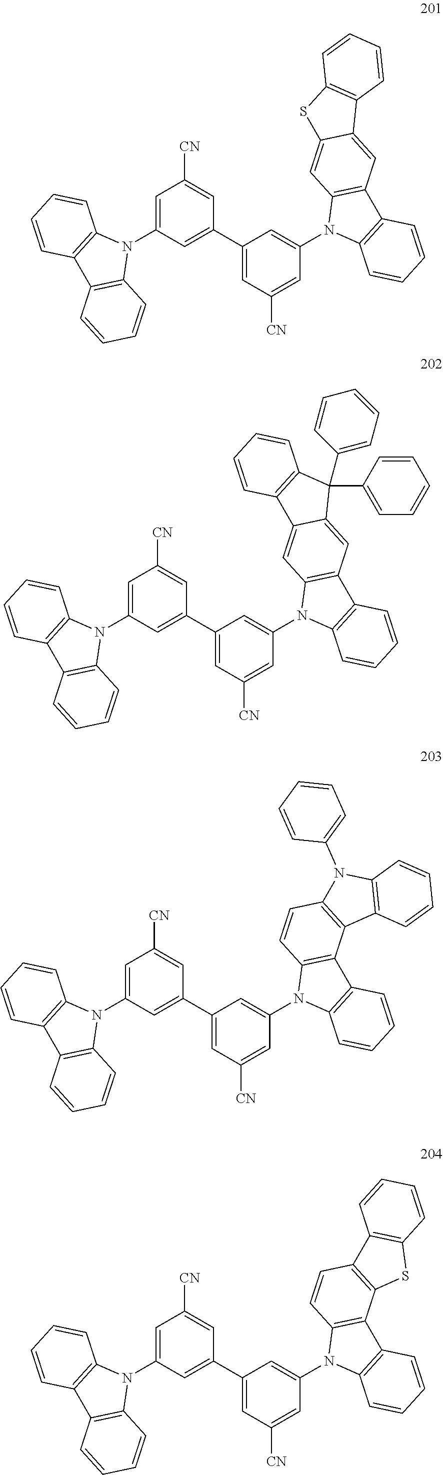 [SMILES: N#Cc1cc(-c2cc(C#N)cc(-n3c4ccccc4c4cc5c(cc43)sc3ccccc35)c2)cc(-n2c3ccccc3c3ccccc32)c1 ; N#Cc1cc(-c2cc(C#N)cc(-n3c4ccccc4c4cc5c(cc43)-c3ccccc3C5(c3ccccc3)c3ccccc3)c2)cc(-n2c3ccccc3c3ccccc32)c1 ; N#Cc1cc(-c2cc(C#N)cc(-n3c4ccccc4c4c5c6ccccc6n(-c6ccccc6)c5ccc43)c2)cc(-n2c3ccccc3c3ccccc32)c1 ; N#Cc1cc(-c2cc(C#N)cc(-n3c4ccccc4c4c5sc6ccccc6c5ccc43)c2)cc(-n2c3ccccc3c3ccccc32)c1]

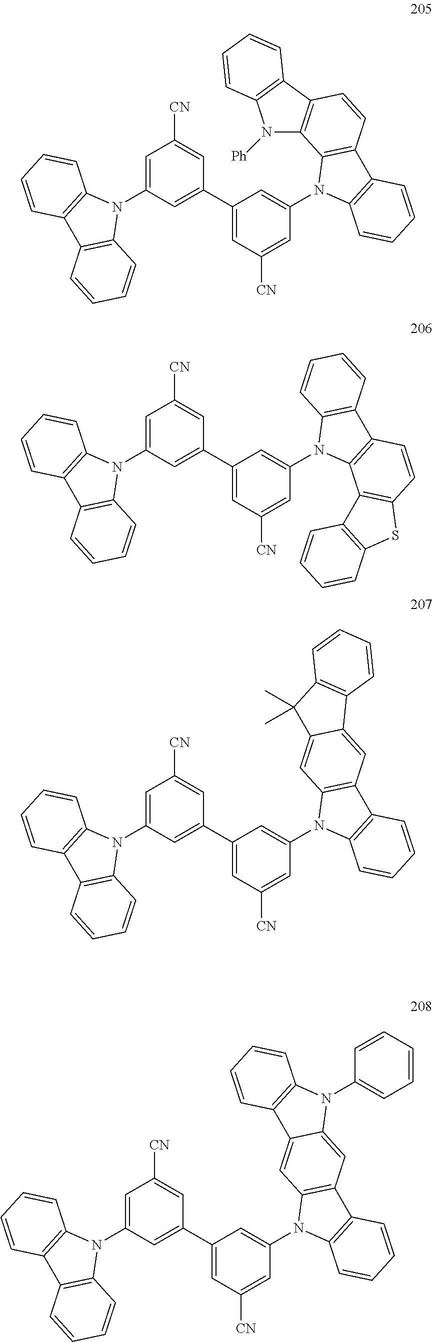

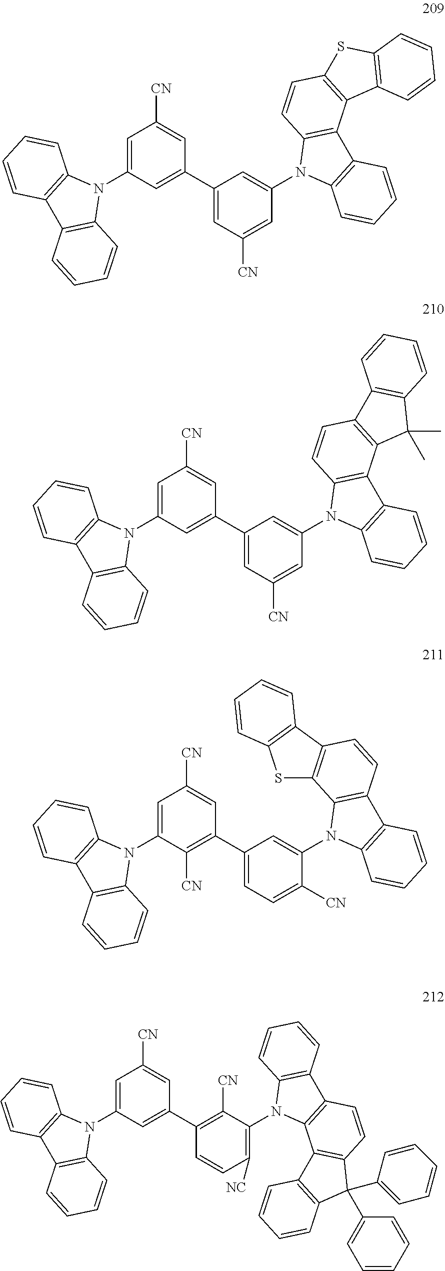 [SMILES: N#Cc1cc(-c2cc(C#N)cc(-n3c4ccccc4c4c5c(ccc43)sc3ccccc35)c2)cc(-n2c3ccccc3c3ccccc32)c1 ; CC1(C)c2ccccc2-c2ccc3c(c21)c1ccccc1n3-c1cc(C#N)cc(-c2cc(C#N)cc(-n3c4ccccc4c4ccccc43)c2)c1 ; N#Cc1cc(-c2ccc(C#N)c(-n3c4ccccc4c4ccc5c6ccccc6sc5c43)c2)c(C#N)c(-n2c3ccccc3c3ccccc32)c1 ; N#Cc1cc(-c2ccc(C#N)c(-n3c4ccccc4c4ccc5c(c43)-c3ccccc3C5(c3ccccc3)c3ccccc3)c2C#N)cc(-n2c3ccccc3c3ccccc32)c1]

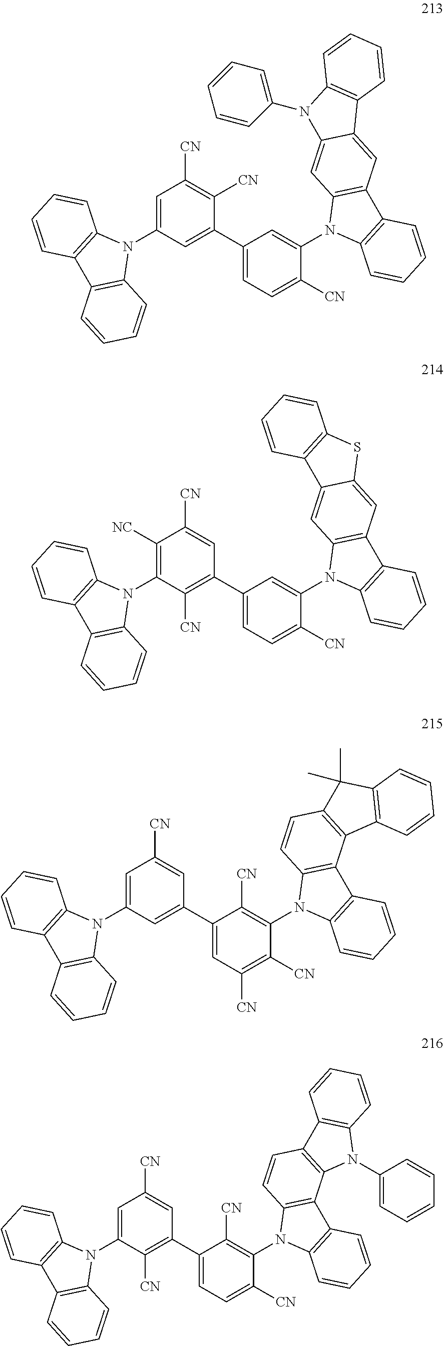 [SMILES: N#Cc1ccc(-c2cc(-n3c4ccccc4c4ccccc43)cc(C#N)c2C#N)cc1-n1c2ccccc2c2cc3c4ccccc4n(-c4ccccc4)c3cc21 ; N#Cc1ccc(-c2cc(C#N)c(C#N)c(-n3c4ccccc4c4ccccc43)c2C#N)cc1-n1c2ccccc2c2cc3sc4ccccc4c3cc21 ; CC1(C)c2ccccc2-c2c1ccc1c2c2ccccc2n1-c1c(C#N)c(C#N)cc(-c2cc(C#N)cc(-n3c4ccccc4c4ccccc43)c2)c1C#N ; N#Cc1cc(-c2ccc(C#N)c(-n3c4ccccc4c4c3ccc3c5ccccc5n(-c5ccccc5)c34)c2C#N)c(C#N)c(-n2c3ccccc3c3ccccc32)c1]

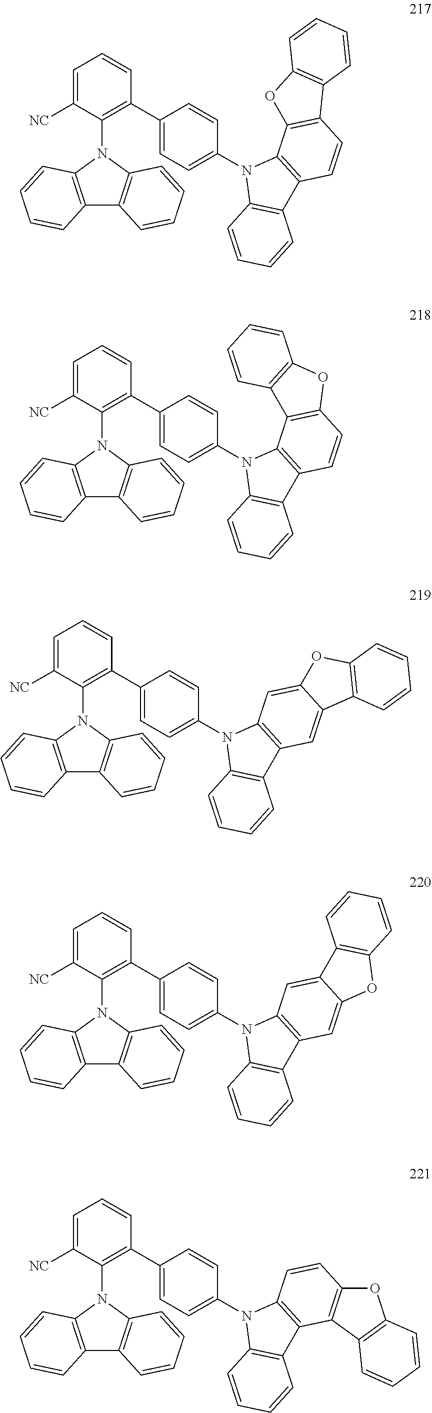 [SMILES: N#Cc1cccc(-c2ccc(-n3c4ccccc4c4ccc5c6ccccc6oc5c43)cc2)c1-n1c2ccccc2c2ccccc21 ; N#Cc1cccc(-c2ccc(-n3c4ccccc4c4ccc5oc6ccccc6c5c43)cc2)c1-n1c2ccccc2c2ccccc21 ; N#Cc1cccc(-c2ccc(-n3c4ccccc4c4cc5c(cc43)oc3ccccc35)cc2)c1-n1c2ccccc2c2ccccc21 ; N#Cc1cccc(-c2ccc(-n3c4ccccc4c4cc5oc6ccccc6c5cc43)cc2)c1-n1c2ccccc2c2ccccc21 ; N#Cc1cccc(-c2ccc(-n3c4ccccc4c4c5c(ccc43)oc3ccccc35)cc2)c1-n1c2ccccc2c2ccccc21]

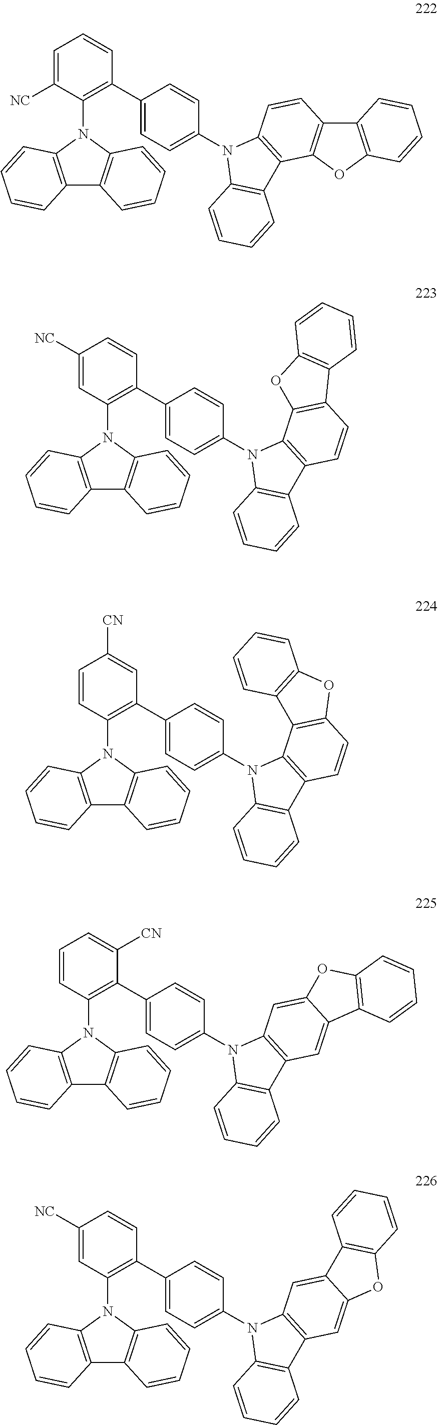 [SMILES: N#Cc1cccc(-c2ccc(-n3c4ccccc4c4c5oc6ccccc6c5ccc43)cc2)c1-n1c2ccccc2c2ccccc21 ; N#Cc1ccc(-c2ccc(-n3c4ccccc4c4ccc5c6ccccc6oc5c43)cc2)c(-n2c3ccccc3c3ccccc32)c1 ; N#Cc1ccc(-n2c3ccccc3c3ccccc32)c(-c2ccc(-n3c4ccccc4c4ccc5oc6ccccc6c5c43)cc2)c1 ; N#Cc1cccc(-n2c3ccccc3c3ccccc32)c1-c1ccc(-n2c3ccccc3c3cc4c(cc32)oc2ccccc24)cc1 ; N#Cc1ccc(-c2ccc(-n3c4ccccc4c4cc5oc6ccccc6c5cc43)cc2)c(-n2c3ccccc3c3ccccc32)c1]

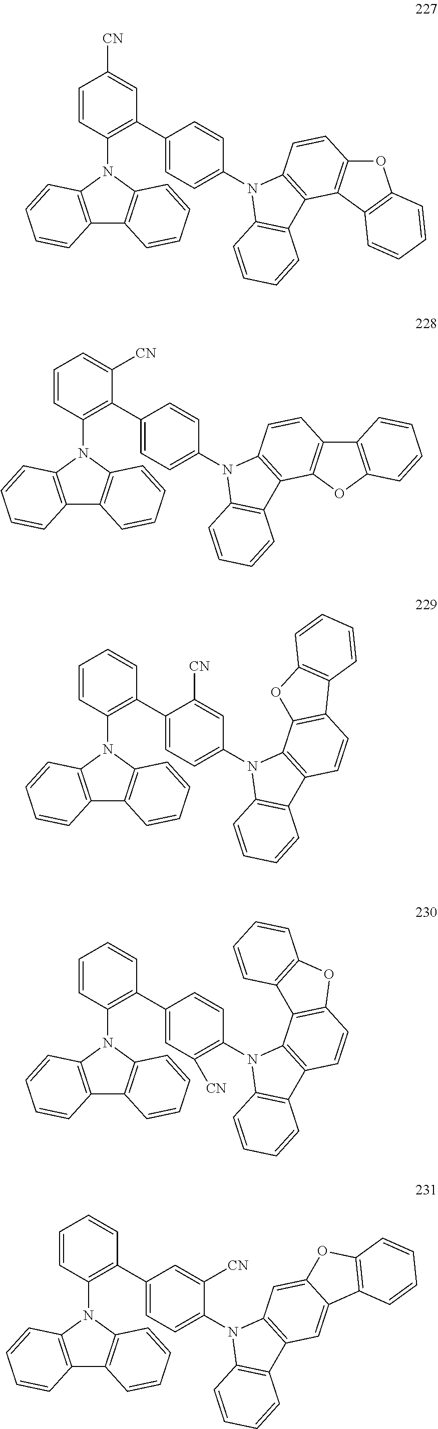 [SMILES: N#Cc1ccc(-n2c3ccccc3c3ccccc32)c(-c2ccc(-n3c4ccccc4c4c5c(ccc43)oc3ccccc35)cc2)c1 ; N#Cc1cccc(-n2c3ccccc3c3ccccc32)c1-c1ccc(-n2c3ccccc3c3c4oc5ccccc5c4ccc32)cc1 ; N#Cc1cc(-n2c3ccccc3c3ccc4c5ccccc5oc4c32)ccc1-c1ccccc1-n1c2ccccc2c2ccccc21 ; N#Cc1cc(-c2ccccc2-n2c3ccccc3c3ccccc32)ccc1-n1c2ccccc2c2ccc3oc4ccccc4c3c21 ; N#Cc1cc(-c2ccccc2-n2c3ccccc3c3ccccc32)ccc1-n1c2ccccc2c2cc3c(cc21)oc1ccccc13]

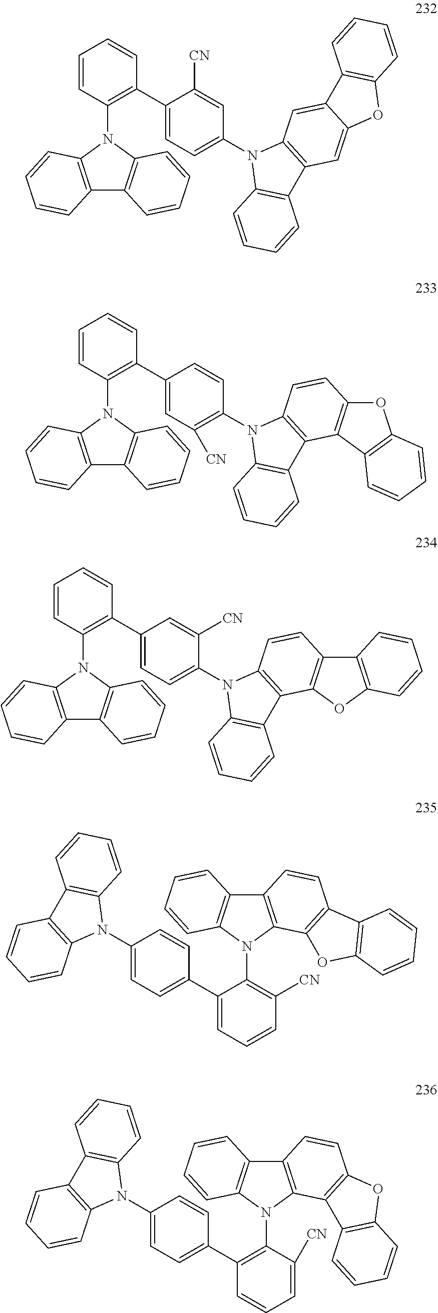 [SMILES: N#Cc1cc(-n2c3ccccc3c3cc4oc5ccccc5c4cc32)ccc1-c1ccccc1-n1c2ccccc2c2ccccc21 ; N#Cc1cc(-c2ccccc2-n2c3ccccc3c3ccccc32)ccc1-n1c2ccccc2c2c3c(ccc21)oc1ccccc13 ; N#Cc1cc(-c2ccccc2-n2c3ccccc3c3ccccc32)ccc1-n1c2ccccc2c2c3oc4ccccc4c3ccc21 ; N#Cc1cccc(-c2ccc(-n3c4ccccc4c4ccccc43)cc2)c1-n1c2ccccc2c2ccc3c4ccccc4oc3c21 ; N#Cc1cccc(-c2ccc(-n3c4ccccc4c4ccccc43)cc2)c1-n1c2ccccc2c2ccc3oc4ccccc4c3c21]

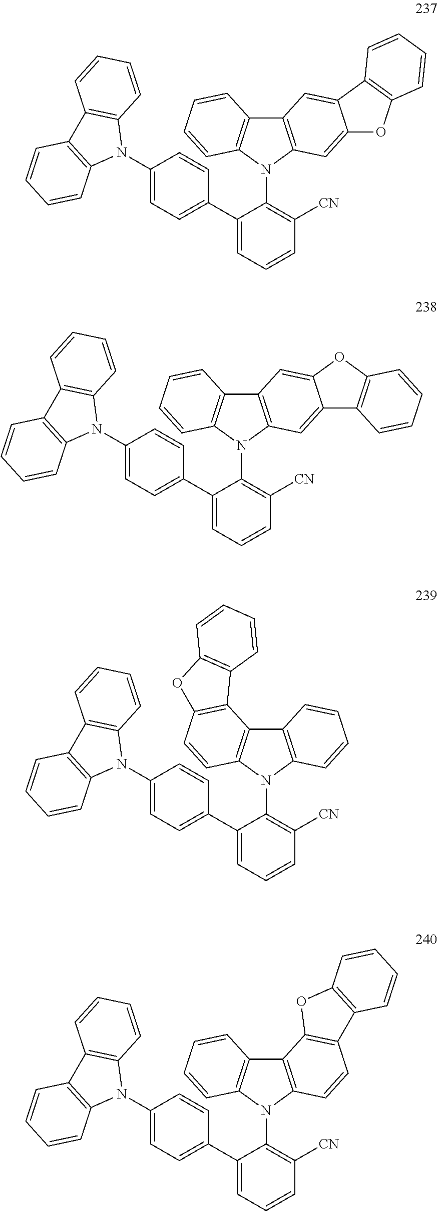 [SMILES: N#Cc1cccc(-c2ccc(-n3c4ccccc4c4ccccc43)cc2)c1-n1c2ccccc2c2cc3c(cc21)oc1ccccc13 ; N#Cc1cccc(-c2ccc(-n3c4ccccc4c4ccccc43)cc2)c1-n1c2ccccc2c2cc3oc4ccccc4c3cc21 ; N#Cc1cccc(-c2ccc(-n3c4ccccc4c4ccccc43)cc2)c1-n1c2ccccc2c2c3c(ccc21)oc1ccccc13 ; N#Cc1cccc(-c2ccc(-n3c4ccccc4c4ccccc43)cc2)c1-n1c2ccccc2c2c3oc4ccccc4c3ccc21]

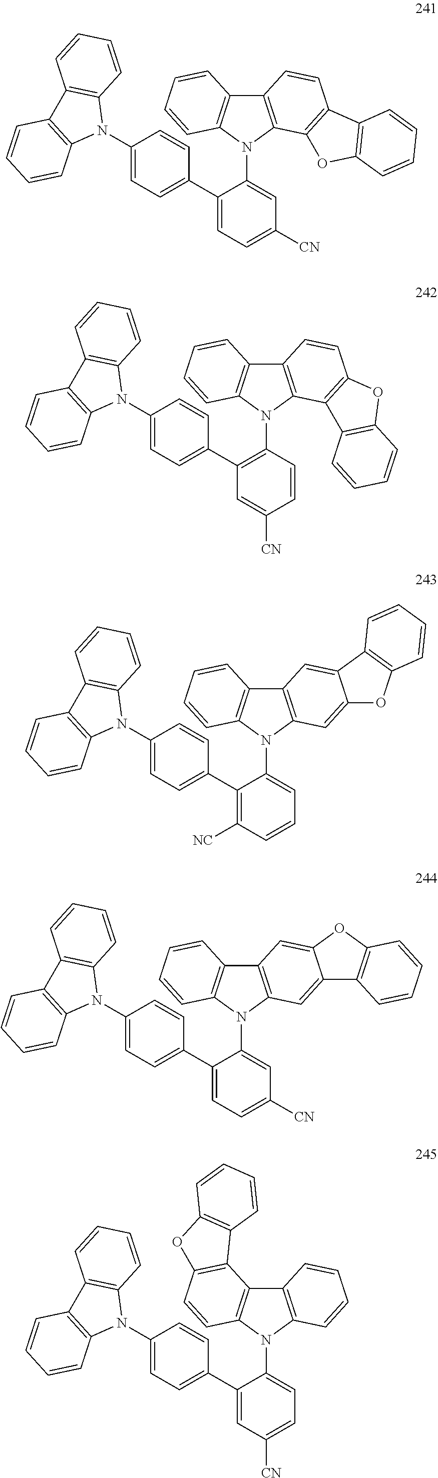 [SMILES: N#Cc1ccc(-c2ccc(-n3c4ccccc4c4ccccc43)cc2)c(-n2c3ccccc3c3ccc4c5ccccc5oc4c32)c1 ; N#Cc1ccc(-n2c3ccccc3c3ccc4oc5ccccc5c4c32)c(-c2ccc(-n3c4ccccc4c4ccccc43)cc2)c1 ; N#Cc1cccc(-n2c3ccccc3c3cc4c(cc32)oc2ccccc24)c1-c1ccc(-n2c3ccccc3c3ccccc32)cc1 ; N#Cc1ccc(-c2ccc(-n3c4ccccc4c4ccccc43)cc2)c(-n2c3ccccc3c3cc4oc5ccccc5c4cc32)c1 ; N#Cc1ccc(-n2c3ccccc3c3c4c(ccc32)oc2ccccc24)c(-c2ccc(-n3c4ccccc4c4ccccc43)cc2)c1]

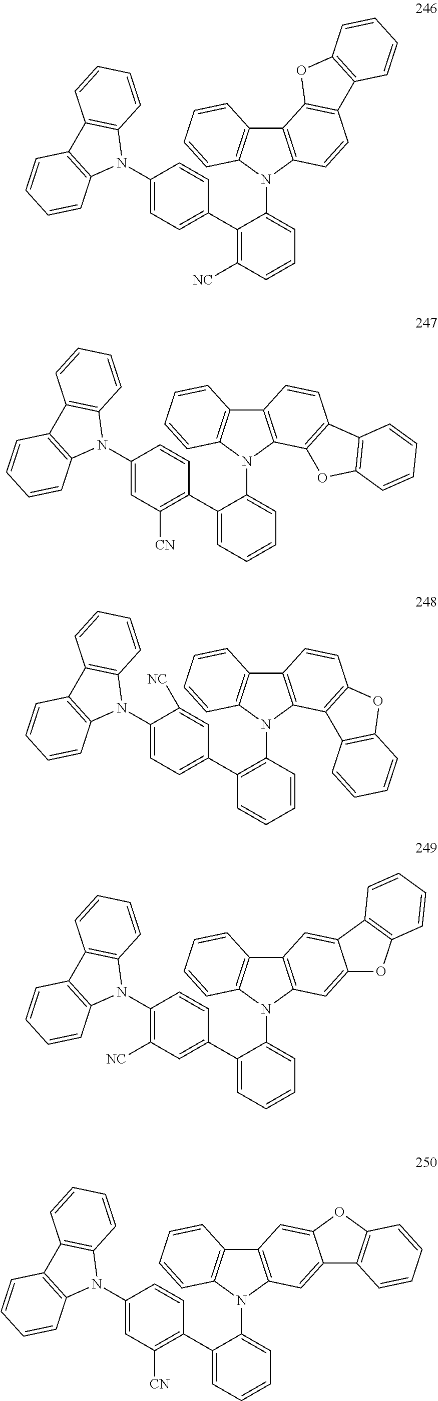 [SMILES: N#Cc1cccc(-n2c3ccccc3c3c4oc5ccccc5c4ccc32)c1-c1ccc(-n2c3ccccc3c3ccccc32)cc1 ; N#Cc1cc(-n2c3ccccc3c3ccccc32)ccc1-c1ccccc1-n1c2ccccc2c2ccc3c4ccccc4oc3c21 ; N#Cc1cc(-c2ccccc2-n2c3ccccc3c3ccc4oc5ccccc5c4c32)ccc1-n1c2ccccc2c2ccccc21 ; N#Cc1cc(-c2ccccc2-n2c3ccccc3c3cc4c(cc32)oc2ccccc24)ccc1-n1c2ccccc2c2ccccc21 ; N#Cc1cc(-n2c3ccccc3c3ccccc32)ccc1-c1ccccc1-n1c2ccccc2c2cc3oc4ccccc4c3cc21]

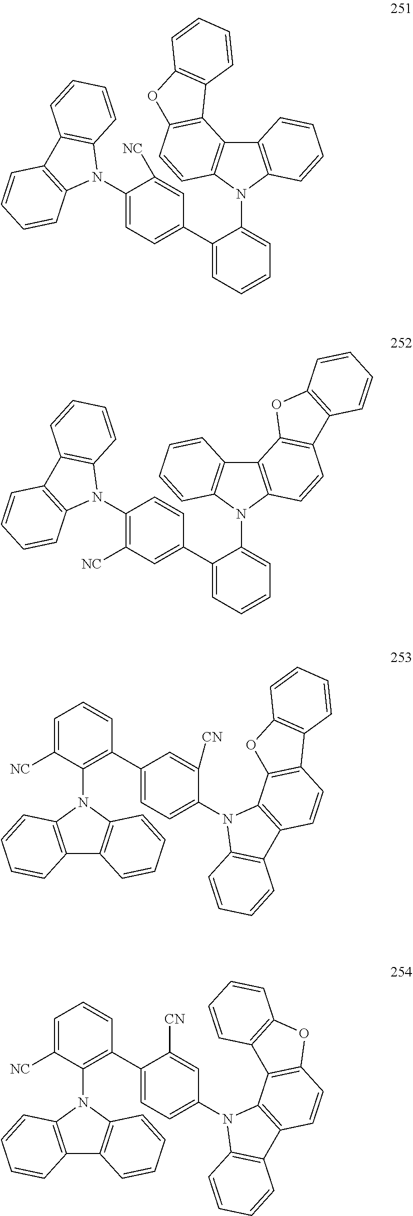 [SMILES: N#Cc1cc(-c2ccccc2-n2c3ccccc3c3c4c(ccc32)oc2ccccc24)ccc1-n1c2ccccc2c2ccccc21 ; N#Cc1cc(-c2ccccc2-n2c3ccccc3c3c4oc5ccccc5c4ccc32)ccc1-n1c2ccccc2c2ccccc21 ; N#Cc1cc(-c2cccc(C#N)c2-n2c3ccccc3c3ccccc32)ccc1-n1c2ccccc2c2ccc3c4ccccc4oc3c21 ; N#Cc1cc(-n2c3ccccc3c3ccc4oc5ccccc5c4c32)ccc1-c1cccc(C#N)c1-n1c2ccccc2c2ccccc21]

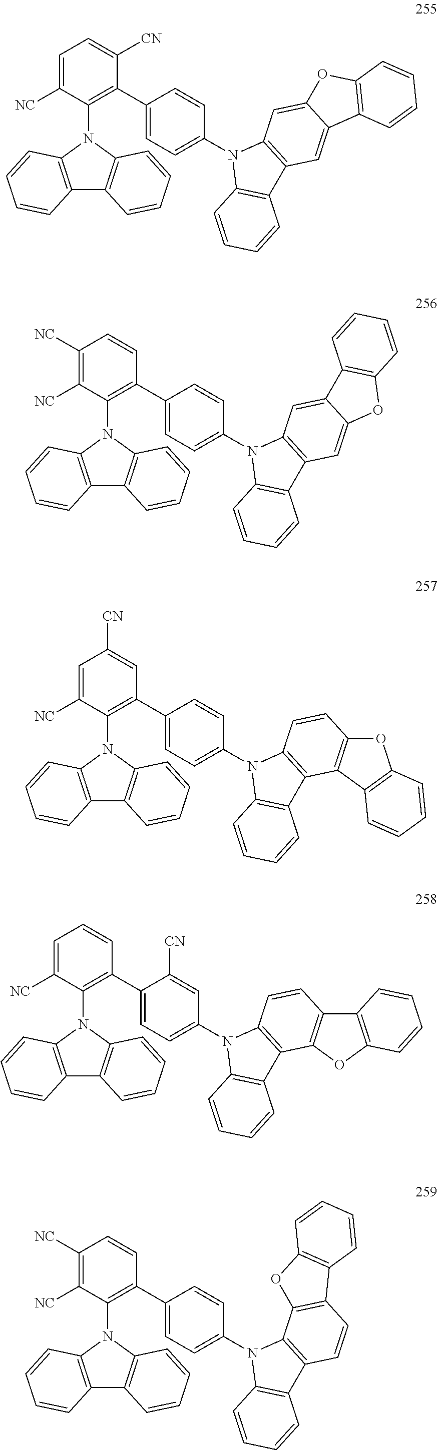 [SMILES: N#Cc1ccc(C#N)c(-n2c3ccccc3c3ccccc32)c1-c1ccc(-n2c3ccccc3c3cc4c(cc32)oc2ccccc24)cc1 ; N#Cc1ccc(-c2ccc(-n3c4ccccc4c4cc5oc6ccccc6c5cc43)cc2)c(-n2c3ccccc3c3ccccc32)c1C#N ; N#Cc1cc(C#N)c(-n2c3ccccc3c3ccccc32)c(-c2ccc(-n3c4ccccc4c4c5c(ccc43)oc3ccccc35)cc2)c1 ; N#Cc1cc(-n2c3ccccc3c3c4oc5ccccc5c4ccc32)ccc1-c1cccc(C#N)c1-n1c2ccccc2c2ccccc21 ; N#Cc1ccc(-c2ccc(-n3c4ccccc4c4ccc5c6ccccc6oc5c43)cc2)c(-n2c3ccccc3c3ccccc32)c1C#N]

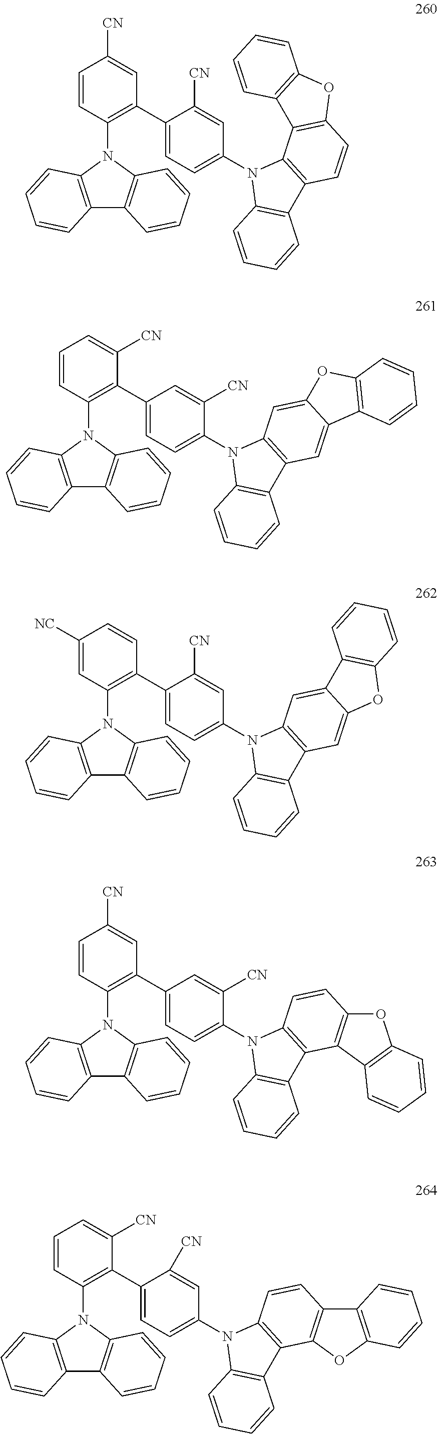 [SMILES: N#Cc1ccc(-n2c3ccccc3c3ccccc32)c(-c2ccc(-n3c4ccccc4c4ccc5oc6ccccc6c5c43)cc2C#N)c1 ; N#Cc1cc(-c2c(C#N)cccc2-n2c3ccccc3c3ccccc32)ccc1-n1c2ccccc2c2cc3c(cc21)oc1ccccc13 ; N#Cc1ccc(-c2ccc(-n3c4ccccc4c4cc5oc6ccccc6c5cc43)cc2C#N)c(-n2c3ccccc3c3ccccc32)c1 ; N#Cc1ccc(-n2c3ccccc3c3ccccc32)c(-c2ccc(-n3c4ccccc4c4c5c(ccc43)oc3ccccc35)c(C#N)c2)c1 ; N#Cc1cc(-n2c3ccccc3c3c4oc5ccccc5c4ccc32)ccc1-c1c(C#N)cccc1-n1c2ccccc2c2ccccc21]

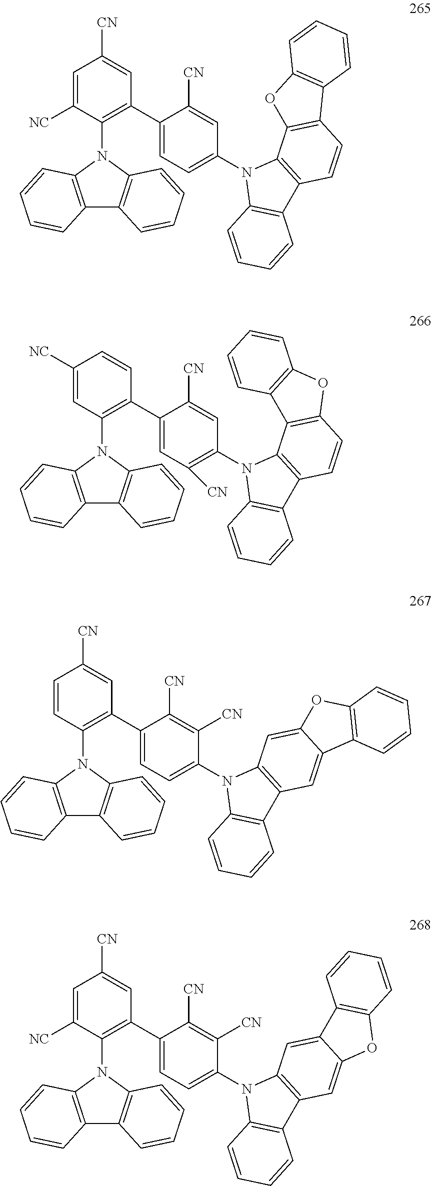 [SMILES: N#Cc1cc(C#N)c(-n2c3ccccc3c3ccccc32)c(-c2ccc(-n3c4ccccc4c4ccc5c6ccccc6oc5c43)cc2C#N)c1 ; N#Cc1ccc(-c2cc(C#N)c(-n3c4ccccc4c4ccc5oc6ccccc6c5c43)cc2C#N)c(-n2c3ccccc3c3ccccc32)c1 ; N#Cc1ccc(-n2c3ccccc3c3ccccc32)c(-c2ccc(-n3c4ccccc4c4cc5c(cc43)oc3ccccc35)c(C#N)c2C#N)c1 ; N#Cc1cc(C#N)c(-n2c3ccccc3c3ccccc32)c(-c2ccc(-n3c4ccccc4c4cc5oc6ccccc6c5cc43)c(C#N)c2C#N)c1]

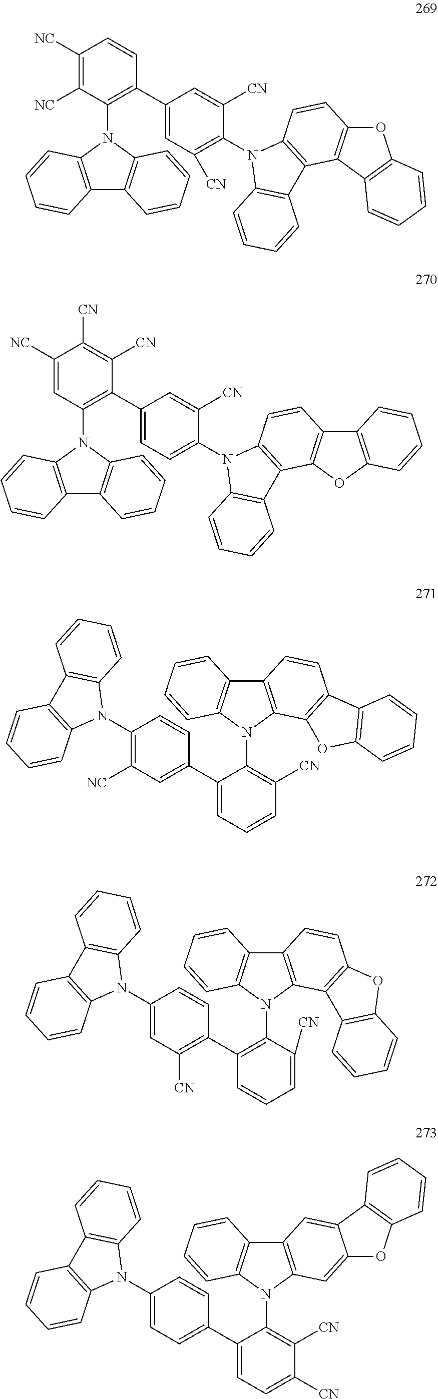 [SMILES: N#Cc1ccc(-c2cc(C#N)c(-n3c4ccccc4c4c5c(ccc43)oc3ccccc35)c(C#N)c2)c(-n2c3ccccc3c3ccccc32)c1C#N ; N#Cc1cc(-c2c(-n3c4ccccc4c4ccccc43)cc(C#N)c(C#N)c2C#N)ccc1-n1c2ccccc2c2c3oc4ccccc4c3ccc21 ; N#Cc1cc(-c2cccc(C#N)c2-n2c3ccccc3c3ccc4c5ccccc5oc4c32)ccc1-n1c2ccccc2c2ccccc21 ; N#Cc1cc(-n2c3ccccc3c3ccccc32)ccc1-c1cccc(C#N)c1-n1c2ccccc2c2ccc3oc4ccccc4c3c21 ; N#Cc1ccc(-c2ccc(-n3c4ccccc4c4ccccc43)cc2)c(-n2c3ccccc3c3cc4c(cc32)oc2ccccc24)c1C#N]

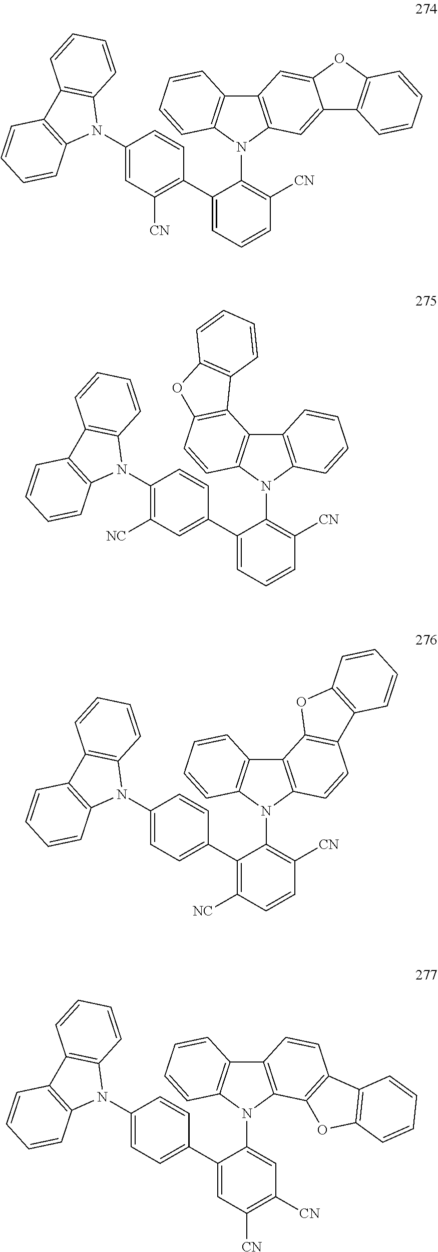 [SMILES: N#Cc1cc(-n2c3ccccc3c3ccccc32)ccc1-c1cccc(C#N)c1-n1c2ccccc2c2cc3oc4ccccc4c3cc21 ; N#Cc1cc(-c2cccc(C#N)c2-n2c3ccccc3c3c4c(ccc32)oc2ccccc24)ccc1-n1c2ccccc2c2ccccc21 ; N#Cc1ccc(C#N)c(-n2c3ccccc3c3c4oc5ccccc5c4ccc32)c1-c1ccc(-n2c3ccccc3c3ccccc32)cc1 ; N#Cc1cc(-c2ccc(-n3c4ccccc4c4ccccc43)cc2)c(-n2c3ccccc3c3ccc4c5ccccc5oc4c32)cc1C#N]

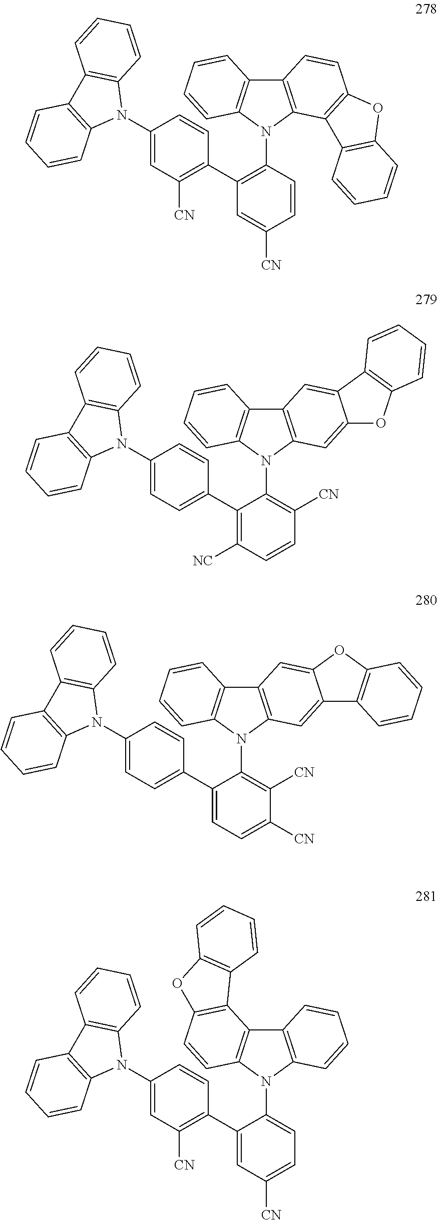 [SMILES: N#Cc1ccc(-n2c3ccccc3c3ccc4oc5ccccc5c4c32)c(-c2ccc(-n3c4ccccc4c4ccccc43)cc2C#N)c1 ; N#Cc1ccc(C#N)c(-n2c3ccccc3c3cc4c(cc32)oc2ccccc24)c1-c1ccc(-n2c3ccccc3c3ccccc32)cc1 ; N#Cc1ccc(-c2ccc(-n3c4ccccc4c4ccccc43)cc2)c(-n2c3ccccc3c3cc4oc5ccccc5c4cc32)c1C#N ; N#Cc1ccc(-n2c3ccccc3c3c4c(ccc32)oc2ccccc24)c(-c2ccc(-n3c4ccccc4c4ccccc43)cc2C#N)c1]

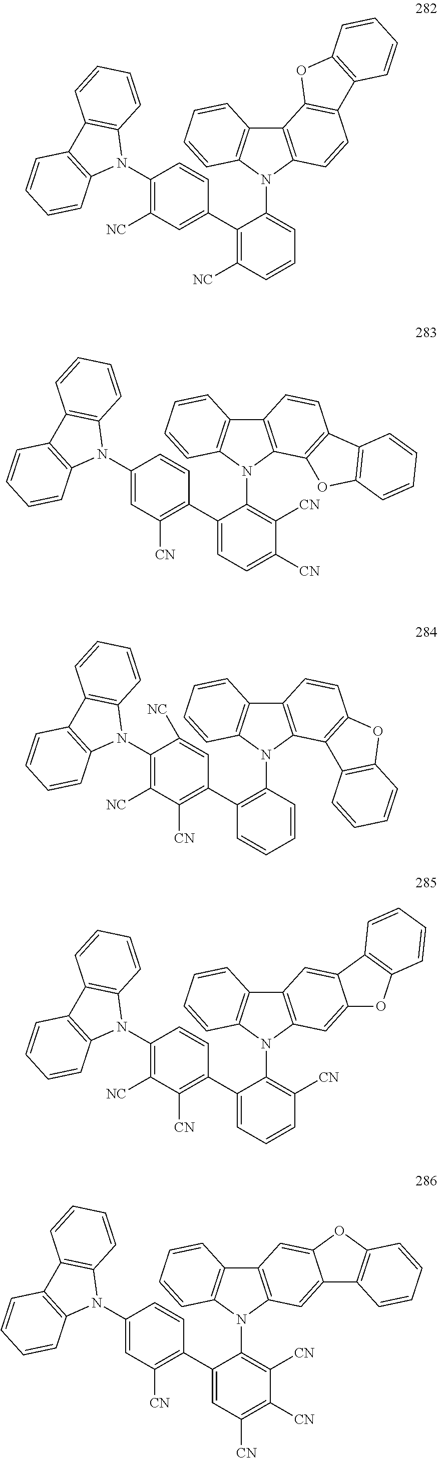 [SMILES: N#Cc1cc(-c2c(C#N)cccc2-n2c3ccccc3c3c4oc5ccccc5c4ccc32)ccc1-n1c2ccccc2c2ccccc21 ; N#Cc1cc(-n2c3ccccc3c3ccccc32)ccc1-c1ccc(C#N)c(C#N)c1-n1c2ccccc2c2ccc3c4ccccc4oc3c21 ; N#Cc1cc(-c2ccccc2-n2c3ccccc3c3ccc4oc5ccccc5c4c32)c(C#N)c(C#N)c1-n1c2ccccc2c2ccccc21 ; N#Cc1cccc(-c2ccc(-n3c4ccccc4c4ccccc43)c(C#N)c2C#N)c1-n1c2ccccc2c2cc3c(cc21)oc1ccccc13 ; N#Cc1cc(-n2c3ccccc3c3ccccc32)ccc1-c1cc(C#N)c(C#N)c(C#N)c1-n1c2ccccc2c2cc3oc4ccccc4c3cc21]

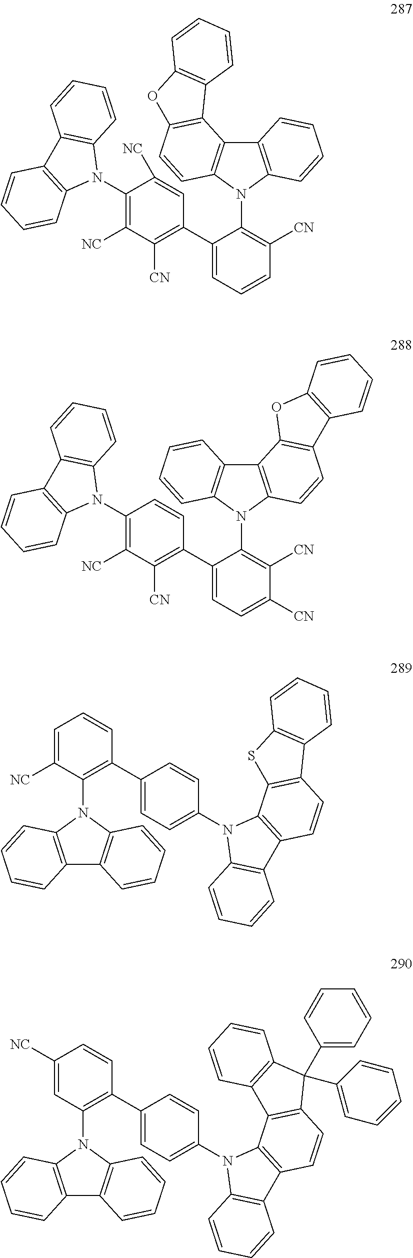 [SMILES: N#Cc1cc(-c2cccc(C#N)c2-n2c3ccccc3c3c4c(ccc32)oc2ccccc24)c(C#N)c(C#N)c1-n1c2ccccc2c2ccccc21 ; N#Cc1ccc(-c2ccc(-n3c4ccccc4c4ccccc43)c(C#N)c2C#N)c(-n2c3ccccc3c3c4oc5ccccc5c4ccc32)c1C#N ; N#Cc1cccc(-c2ccc(-n3c4ccccc4c4ccc5c6ccccc6sc5c43)cc2)c1-n1c2ccccc2c2ccccc21 ; N#Cc1ccc(-c2ccc(-n3c4ccccc4c4ccc5c(c43)-c3ccccc3C5(c3ccccc3)c3ccccc3)cc2)c(-n2c3ccccc3c3ccccc32)c1]

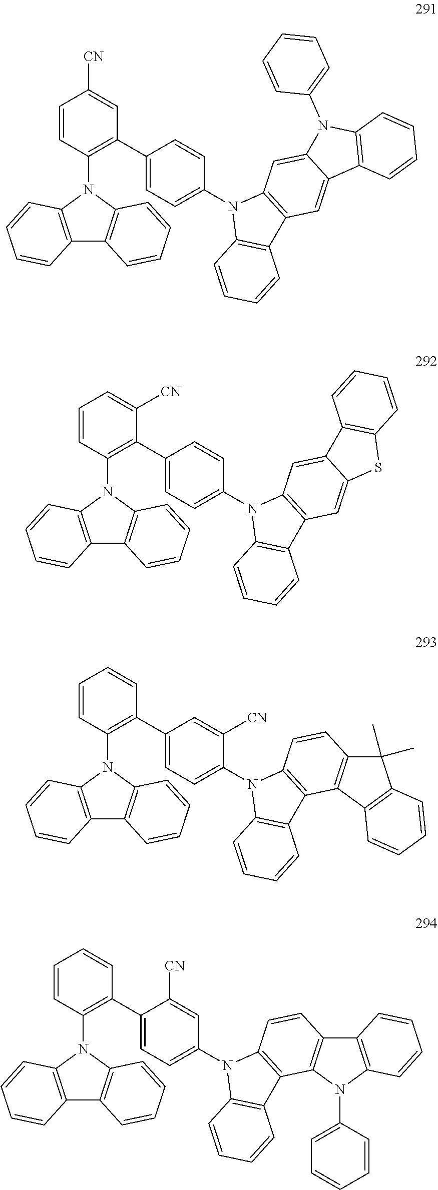 [SMILES: N#Cc1ccc(-n2c3ccccc3c3ccccc32)c(-c2ccc(-n3c4ccccc4c4cc5c6ccccc6n(-c6ccccc6)c5cc43)cc2)c1 ; N#Cc1cccc(-n2c3ccccc3c3ccccc32)c1-c1ccc(-n2c3ccccc3c3cc4sc5ccccc5c4cc32)cc1 ; CC1(C)c2ccccc2-c2c1ccc1c2c2ccccc2n1-c1ccc(-c2ccccc2-n2c3ccccc3c3ccccc32)cc1C#N ; N#Cc1cc(-n2c3ccccc3c3c2ccc2c4ccccc4n(-c4ccccc4)c23)ccc1-c1ccccc1-n1c2ccccc2c2ccccc21]

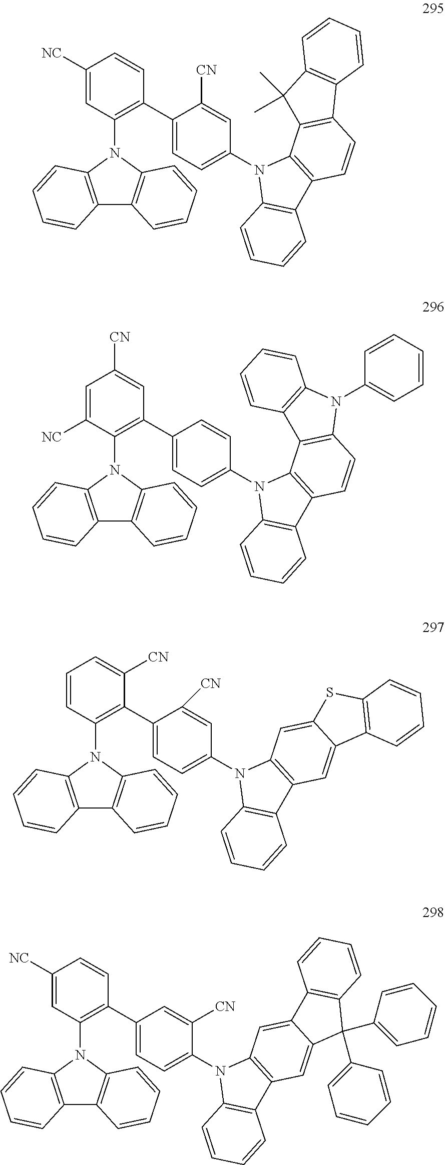 [SMILES: CC1(C)c2ccccc2-c2ccc3c4ccccc4n(-c4ccc(-c5ccc(C#N)cc5-n5c6ccccc6c6ccccc65)c(C#N)c4)c3c21 ; N#Cc1cc(C#N)c(-n2c3ccccc3c3ccccc32)c(-c2ccc(-n3c4ccccc4c4ccc5c(c6ccccc6n5-c5ccccc5)c43)cc2)c1 ; N#Cc1cc(-n2c3ccccc3c3cc4c(cc32)sc2ccccc24)ccc1-c1c(C#N)cccc1-n1c2ccccc2c2ccccc21 ; N#Cc1ccc(-c2ccc(-n3c4ccccc4c4cc5c(cc43)-c3ccccc3C5(c3ccccc3)c3ccccc3)c(C#N)c2)c(-n2c3ccccc3c3ccccc32)c1]

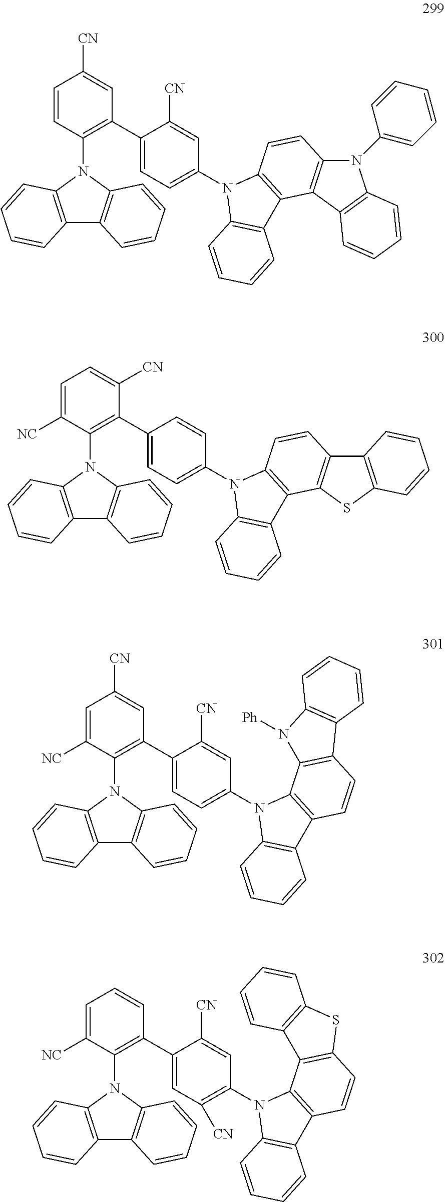 [SMILES: N#Cc1ccc(-n2c3ccccc3c3ccccc32)c(-c2ccc(-n3c4ccccc4c4c5c6ccccc6n(-c6ccccc6)c5ccc43)cc2C#N)c1 ; N#Cc1ccc(C#N)c(-n2c3ccccc3c3ccccc32)c1-c1ccc(-n2c3ccccc3c3c4sc5ccccc5c4ccc32)cc1 ; N#Cc1cc(C#N)c(-n2c3ccccc3c3ccccc32)c(-c2ccc(-n3c4ccccc4c4ccc5c6ccccc6n(-c6ccccc6)c5c43)cc2C#N)c1 ; N#Cc1cc(-n2c3ccccc3c3ccc4sc5ccccc5c4c32)c(C#N)cc1-c1cccc(C#N)c1-n1c2ccccc2c2ccccc21]

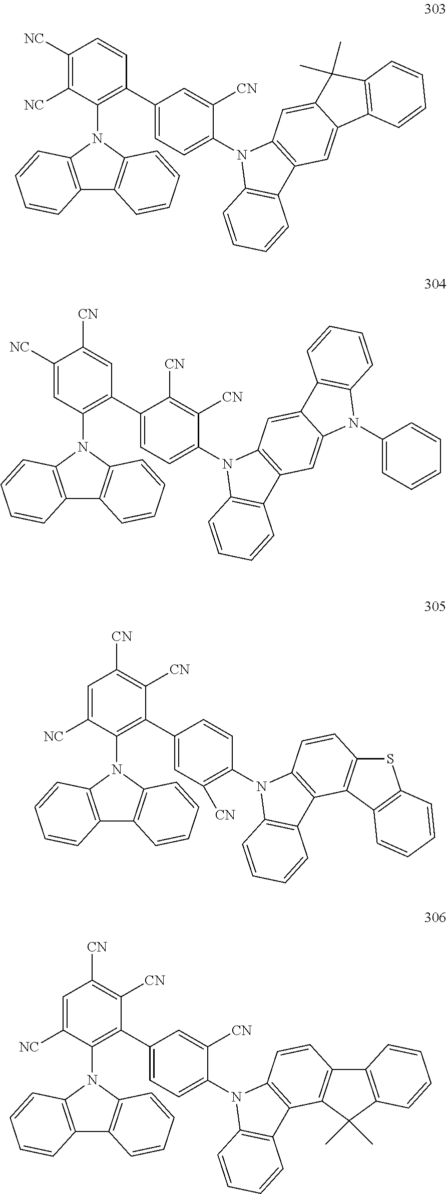 [SMILES: CC1(C)c2ccccc2-c2cc3c4ccccc4n(-c4ccc(-c5ccc(C#N)c(C#N)c5-n5c6ccccc6c6ccccc65)cc4C#N)c3cc21 ; N#Cc1cc(-c2ccc(-n3c4ccccc4c4cc5c(cc43)c3ccccc3n5-c3ccccc3)c(C#N)c2C#N)c(-n2c3ccccc3c3ccccc32)cc1C#N ; N#Cc1cc(-c2c(C#N)c(C#N)cc(C#N)c2-n2c3ccccc3c3ccccc32)ccc1-n1c2ccccc2c2c3c(ccc21)sc1ccccc13 ; CC1(C)c2ccccc2-c2ccc3c(c21)c1ccccc1n3-c1ccc(-c2c(C#N)c(C#N)cc(C#N)c2-n2c3ccccc3c3ccccc32)cc1C#N]

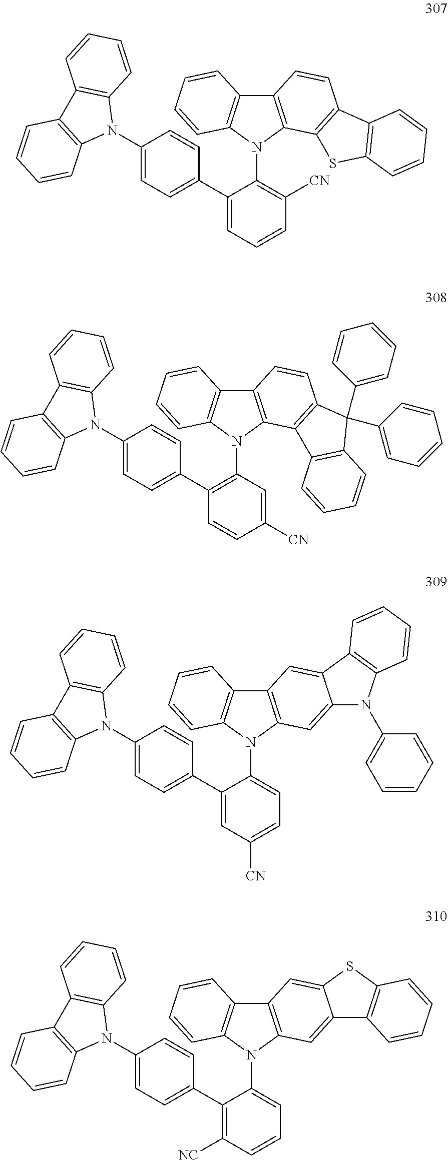 [SMILES: N#Cc1cccc(-c2ccc(-n3c4ccccc4c4ccccc43)cc2)c1-n1c2ccccc2c2ccc3c4ccccc4sc3c21 ; N#Cc1ccc(-c2ccc(-n3c4ccccc4c4ccccc43)cc2)c(-n2c3ccccc3c3ccc4c(c32)-c2ccccc2C4(c2ccccc2)c2ccccc2)c1 ; N#Cc1ccc(-n2c3ccccc3c3cc4c5ccccc5n(-c5ccccc5)c4cc32)c(-c2ccc(-n3c4ccccc4c4ccccc43)cc2)c1 ; N#Cc1cccc(-n2c3ccccc3c3cc4sc5ccccc5c4cc32)c1-c1ccc(-n2c3ccccc3c3ccccc32)cc1]

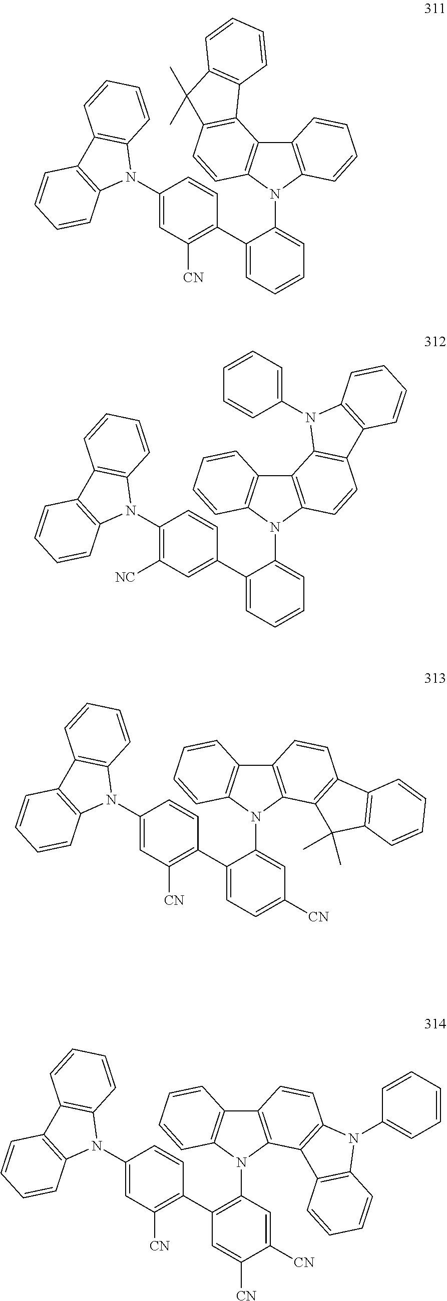 [SMILES: CC1(C)c2ccccc2-c2c1ccc1c2c2ccccc2n1-c1ccccc1-c1ccc(-n2c3ccccc3c3ccccc32)cc1C#N ; N#Cc1cc(-c2ccccc2-n2c3ccccc3c3c2ccc2c4ccccc4n(-c4ccccc4)c23)ccc1-n1c2ccccc2c2ccccc21 ; CC1(C)c2ccccc2-c2ccc3c4ccccc4n(-c4cc(C#N)ccc4-c4ccc(-n5c6ccccc6c6ccccc65)cc4C#N)c3c21 ; N#Cc1cc(-c2ccc(-n3c4ccccc4c4ccccc43)cc2C#N)c(-n2c3ccccc3c3ccc4c(c5ccccc5n4-c4ccccc4)c32)cc1C#N]

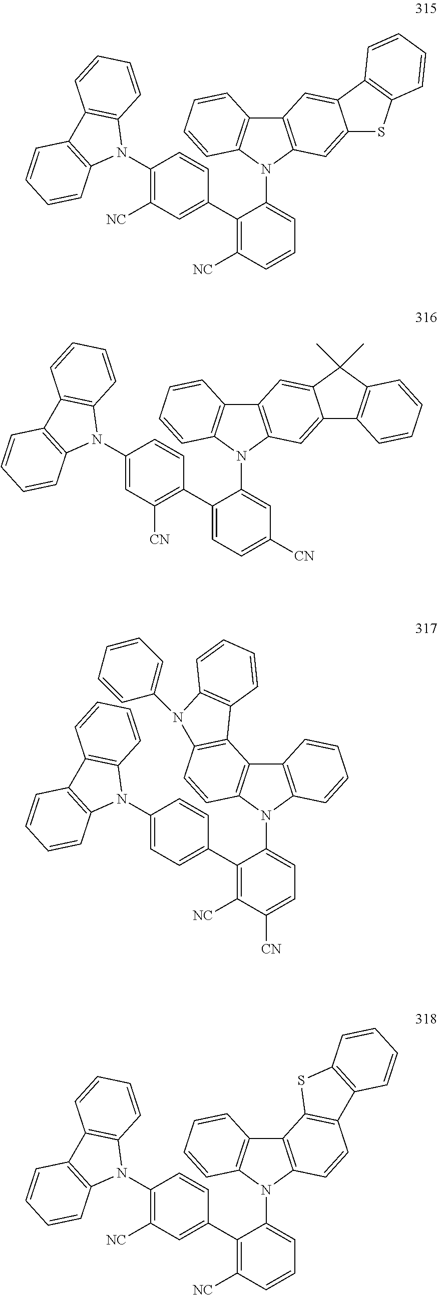 [SMILES: N#Cc1cc(-c2c(C#N)cccc2-n2c3ccccc3c3cc4c(cc32)sc2ccccc24)ccc1-n1c2ccccc2c2ccccc21 ; CC1(C)c2ccccc2-c2cc3c(cc21)c1ccccc1n3-c1cc(C#N)ccc1-c1ccc(-n2c3ccccc3c3ccccc32)cc1C#N ; N#Cc1ccc(-n2c3ccccc3c3c4c5ccccc5n(-c5ccccc5)c4ccc32)c(-c2ccc(-n3c4ccccc4c4ccccc43)cc2)c1C#N ; N#Cc1cc(-c2c(C#N)cccc2-n2c3ccccc3c3c4sc5ccccc5c4ccc32)ccc1-n1c2ccccc2c2ccccc21]

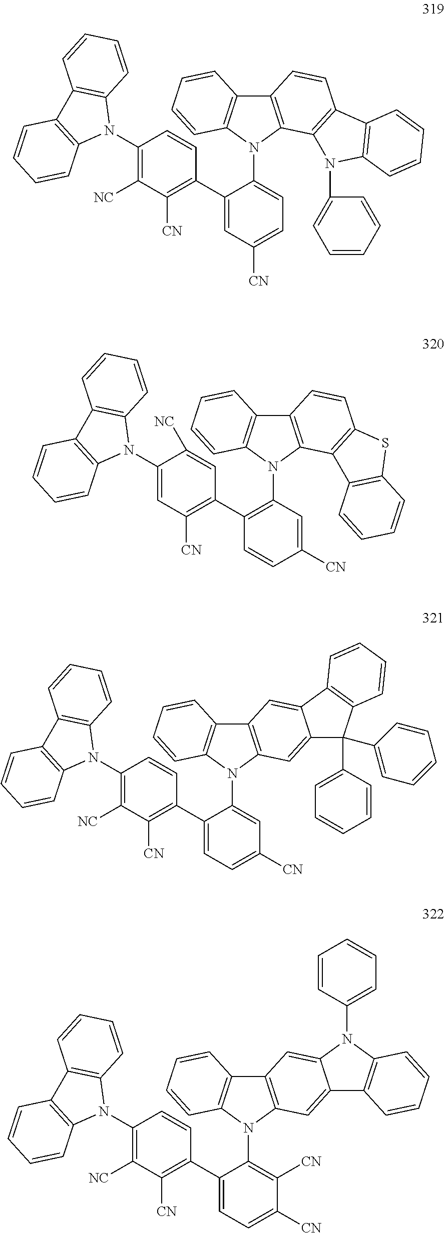 [SMILES: N#Cc1ccc(-n2c3ccccc3c3ccc4c5ccccc5n(-c5ccccc5)c4c32)c(-c2ccc(-n3c4ccccc4c4ccccc43)c(C#N)c2C#N)c1 ; N#Cc1ccc(-c2cc(C#N)c(-n3c4ccccc4c4ccccc43)cc2C#N)c(-n2c3ccccc3c3ccc4sc5ccccc5c4c32)c1 ; N#Cc1ccc(-c2ccc(-n3c4ccccc4c4ccccc43)c(C#N)c2C#N)c(-n2c3ccccc3c3cc4c(cc32)C(c2ccccc2)(c2ccccc2)c2ccccc2-4)c1 ; N#Cc1ccc(-c2ccc(-n3c4ccccc4c4ccccc43)c(C#N)c2C#N)c(-n2c3ccccc3c3cc4c(cc32)c2ccccc2n4-c2ccccc2)c1C#N]

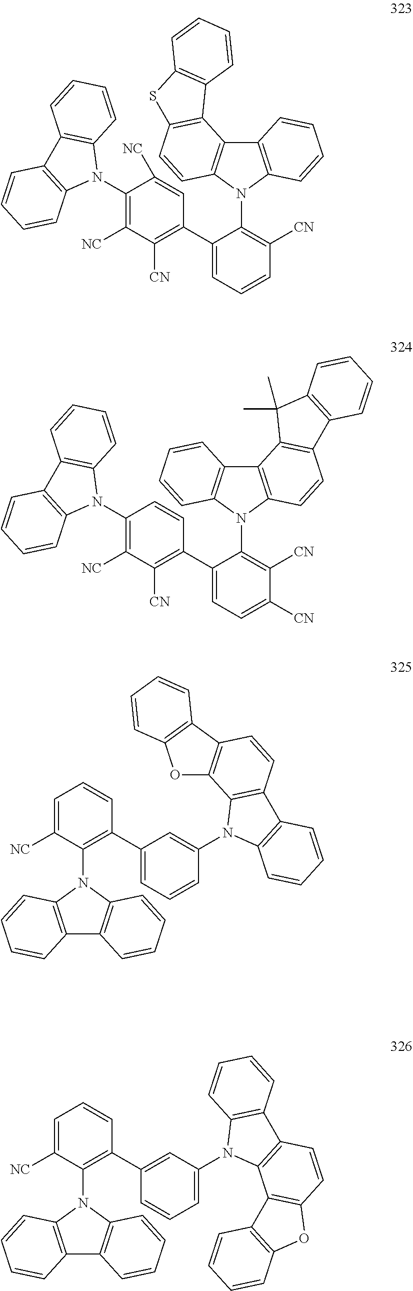 [SMILES: N#Cc1cc(-c2cccc(C#N)c2-n2c3ccccc3c3c4c(ccc32)sc2ccccc24)c(C#N)c(C#N)c1-n1c2ccccc2c2ccccc21 ; CC1(C)c2ccccc2-c2ccc3c(c21)c1ccccc1n3-c1c(-c2ccc(-n3c4ccccc4c4ccccc43)c(C#N)c2C#N)ccc(C#N)c1C#N ; N#Cc1cccc(-c2cccc(-n3c4ccccc4c4ccc5c6ccccc6oc5c43)c2)c1-n1c2ccccc2c2ccccc21 ; N#Cc1cccc(-c2cccc(-n3c4ccccc4c4ccc5oc6ccccc6c5c43)c2)c1-n1c2ccccc2c2ccccc21]

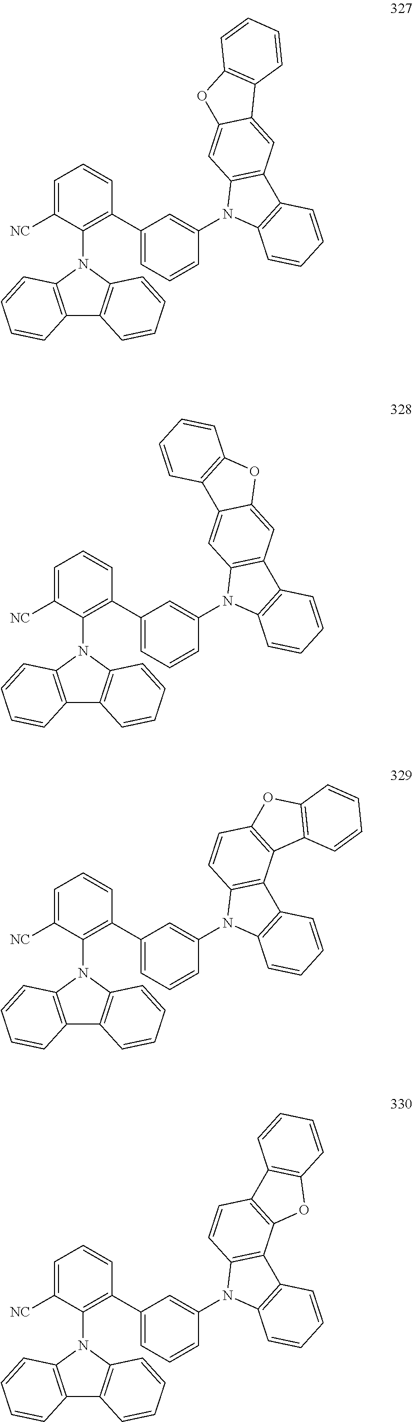 [SMILES: N#Cc1cccc(-c2cccc(-n3c4ccccc4c4cc5c(cc43)oc3ccccc35)c2)c1-n1c2ccccc2c2ccccc21 ; N#Cc1cccc(-c2cccc(-n3c4ccccc4c4cc5oc6ccccc6c5cc43)c2)c1-n1c2ccccc2c2ccccc21 ; N#Cc1cccc(-c2cccc(-n3c4ccccc4c4c5c(ccc43)oc3ccccc35)c2)c1-n1c2ccccc2c2ccccc21 ; N#Cc1cccc(-c2cccc(-n3c4ccccc4c4c5oc6ccccc6c5ccc43)c2)c1-n1c2ccccc2c2ccccc21]

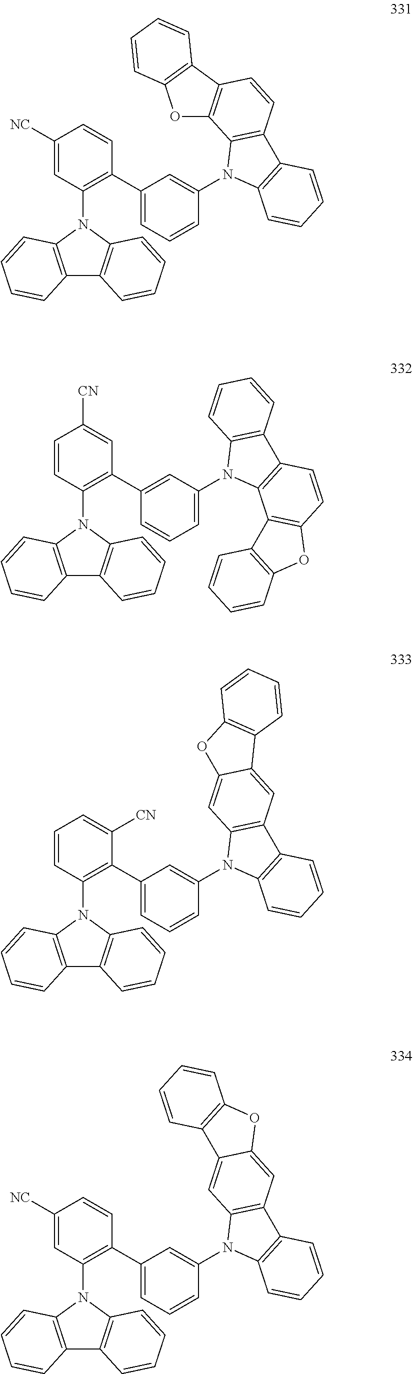 [SMILES: N#Cc1ccc(-c2cccc(-n3c4ccccc4c4ccc5c6ccccc6oc5c43)c2)c(-n2c3ccccc3c3ccccc32)c1 ; N#Cc1ccc(-n2c3ccccc3c3ccccc32)c(-c2cccc(-n3c4ccccc4c4ccc5oc6ccccc6c5c43)c2)c1 ; N#Cc1cccc(-n2c3ccccc3c3ccccc32)c1-c1cccc(-n2c3ccccc3c3cc4c(cc32)oc2ccccc24)c1 ; N#Cc1ccc(-c2cccc(-n3c4ccccc4c4cc5oc6ccccc6c5cc43)c2)c(-n2c3ccccc3c3ccccc32)c1]

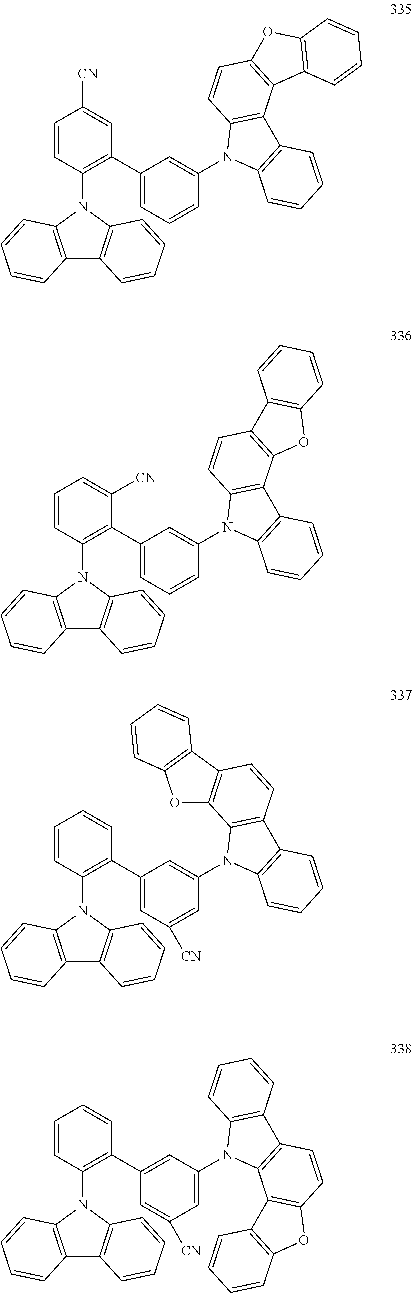 [SMILES: N#Cc1ccc(-n2c3ccccc3c3ccccc32)c(-c2cccc(-n3c4ccccc4c4c5c(ccc43)oc3ccccc35)c2)c1 ; N#Cc1cccc(-n2c3ccccc3c3ccccc32)c1-c1cccc(-n2c3ccccc3c3c4oc5ccccc5c4ccc32)c1 ; N#Cc1cc(-c2ccccc2-n2c3ccccc3c3ccccc32)cc(-n2c3ccccc3c3ccc4c5ccccc5oc4c32)c1 ; N#Cc1cc(-c2ccccc2-n2c3ccccc3c3ccccc32)cc(-n2c3ccccc3c3ccc4oc5ccccc5c4c32)c1]

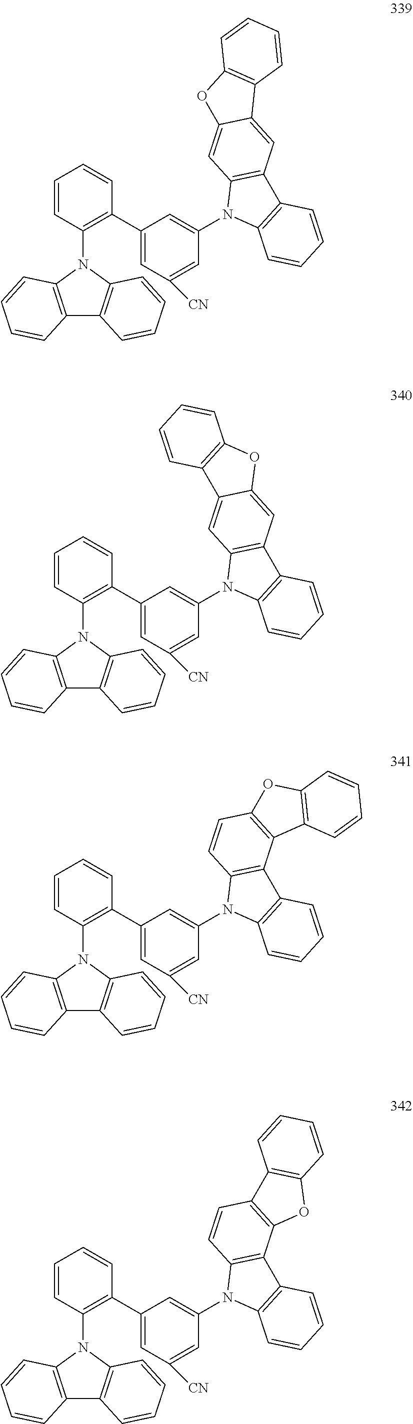 [SMILES: N#Cc1cc(-c2ccccc2-n2c3ccccc3c3ccccc32)cc(-n2c3ccccc3c3cc4c(cc32)oc2ccccc24)c1 ; N#Cc1cc(-c2ccccc2-n2c3ccccc3c3ccccc32)cc(-n2c3ccccc3c3cc4oc5ccccc5c4cc32)c1 ; N#Cc1cc(-c2ccccc2-n2c3ccccc3c3ccccc32)cc(-n2c3ccccc3c3c4c(ccc32)oc2ccccc24)c1 ; N#Cc1cc(-c2ccccc2-n2c3ccccc3c3ccccc32)cc(-n2c3ccccc3c3c4oc5ccccc5c4ccc32)c1]

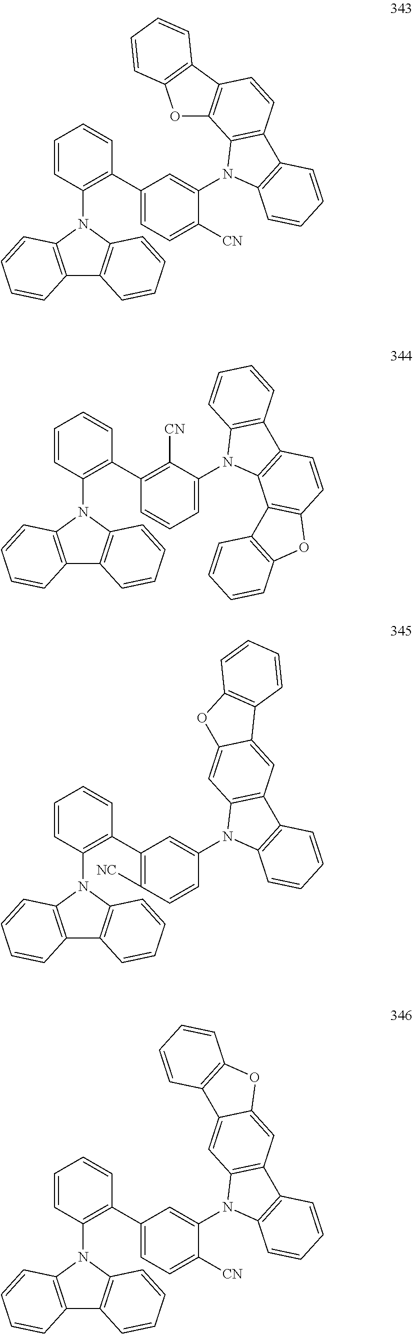

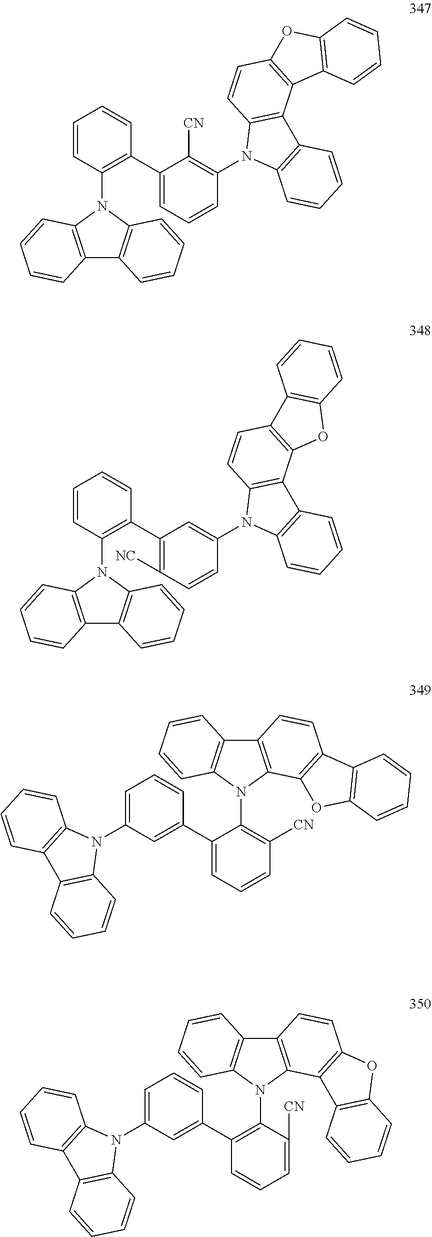 [SMILES: N#Cc1c(-c2ccccc2-n2c3ccccc3c3ccccc32)cccc1-n1c2ccccc2c2c3c(ccc21)oc1ccccc13 ; N#Cc1ccc(-n2c3ccccc3c3c4oc5ccccc5c4ccc32)cc1-c1ccccc1-n1c2ccccc2c2ccccc21 ; N#Cc1cccc(-c2cccc(-n3c4ccccc4c4ccccc43)c2)c1-n1c2ccccc2c2ccc3c4ccccc4oc3c21 ; N#Cc1cccc(-c2cccc(-n3c4ccccc4c4ccccc43)c2)c1-n1c2ccccc2c2ccc3oc4ccccc4c3c21]

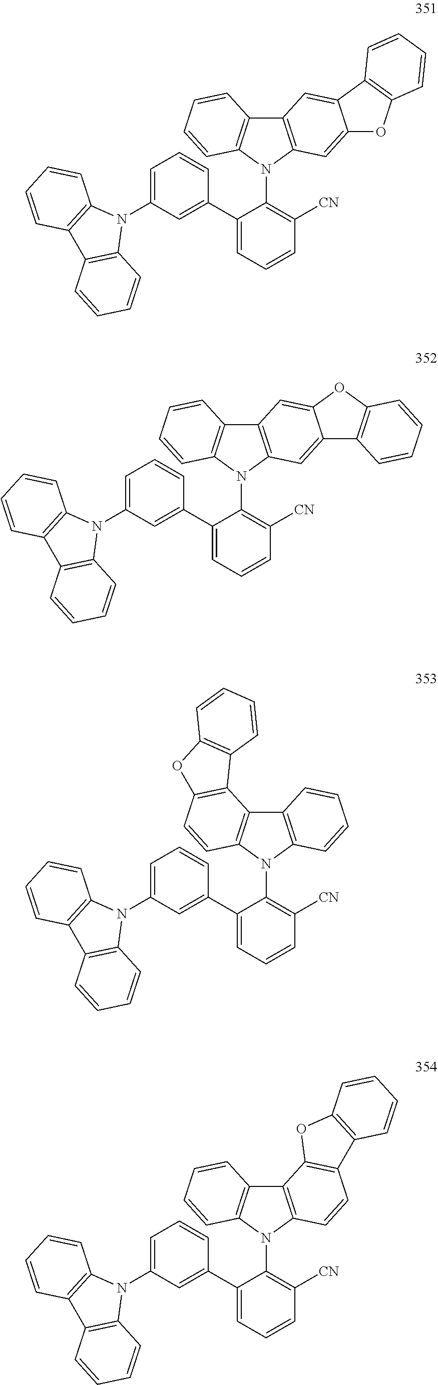 [SMILES: N#Cc1cccc(-c2cccc(-n3c4ccccc4c4ccccc43)c2)c1-n1c2ccccc2c2cc3c(cc21)oc1ccccc13 ; N#Cc1cccc(-c2cccc(-n3c4ccccc4c4ccccc43)c2)c1-n1c2ccccc2c2cc3oc4ccccc4c3cc21 ; N#Cc1cccc(-c2cccc(-n3c4ccccc4c4ccccc43)c2)c1-n1c2ccccc2c2c3c(ccc21)oc1ccccc13 ; N#Cc1cccc(-c2cccc(-n3c4ccccc4c4ccccc43)c2)c1-n1c2ccccc2c2c3oc4ccccc4c3ccc21]

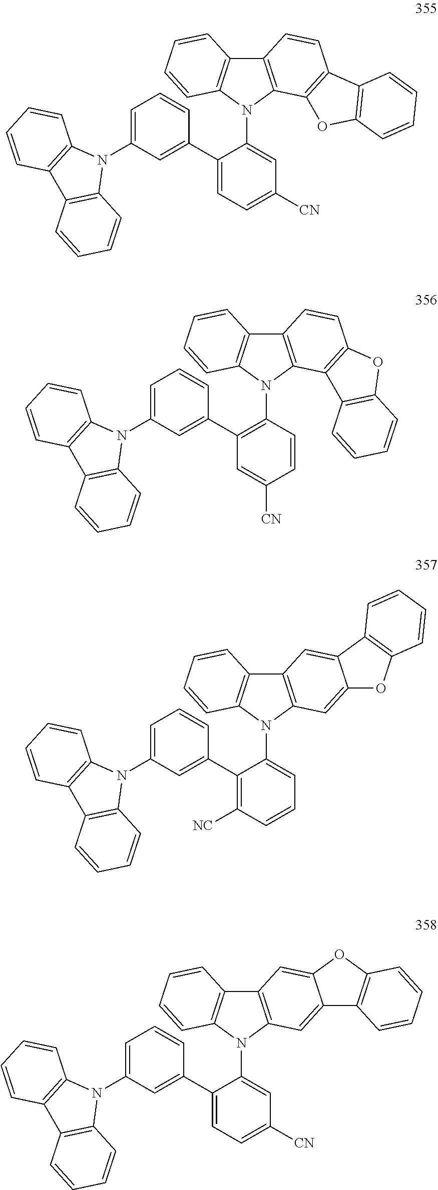 [SMILES: N#Cc1ccc(-c2cccc(-n3c4ccccc4c4ccccc43)c2)c(-n2c3ccccc3c3ccc4c5ccccc5oc4c32)c1 ; N#Cc1ccc(-n2c3ccccc3c3ccc4oc5ccccc5c4c32)c(-c2cccc(-n3c4ccccc4c4ccccc43)c2)c1 ; N#Cc1cccc(-n2c3ccccc3c3cc4c(cc32)oc2ccccc24)c1-c1cccc(-n2c3ccccc3c3ccccc32)c1 ; N#Cc1ccc(-c2cccc(-n3c4ccccc4c4ccccc43)c2)c(-n2c3ccccc3c3cc4oc5ccccc5c4cc32)c1]

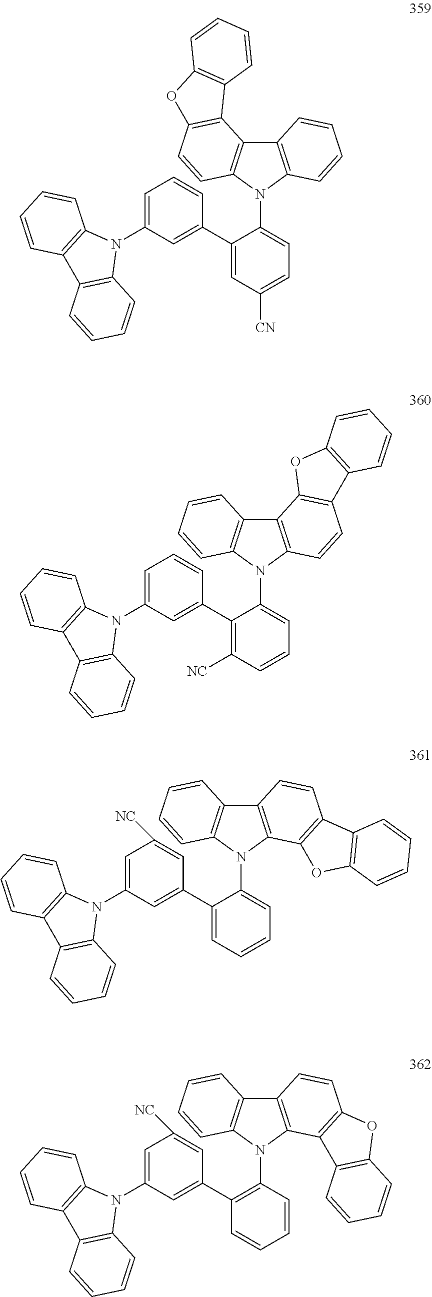 [SMILES: N#Cc1ccc(-n2c3ccccc3c3c4c(ccc32)oc2ccccc24)c(-c2cccc(-n3c4ccccc4c4ccccc43)c2)c1 ; N#Cc1cccc(-n2c3ccccc3c3c4oc5ccccc5c4ccc32)c1-c1cccc(-n2c3ccccc3c3ccccc32)c1 ; N#Cc1cc(-c2ccccc2-n2c3ccccc3c3ccc4c5ccccc5oc4c32)cc(-n2c3ccccc3c3ccccc32)c1 ; N#Cc1cc(-c2ccccc2-n2c3ccccc3c3ccc4oc5ccccc5c4c32)cc(-n2c3ccccc3c3ccccc32)c1]

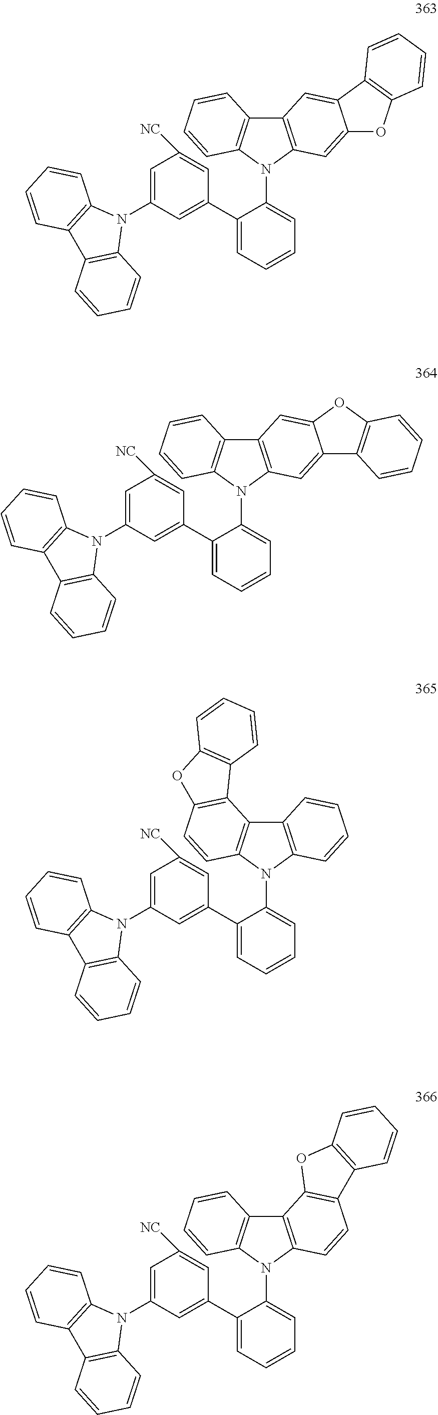 [SMILES: N#Cc1cc(-c2ccccc2-n2c3ccccc3c3cc4c(cc32)oc2ccccc24)cc(-n2c3ccccc3c3ccccc32)c1 ; N#Cc1cc(-c2ccccc2-n2c3ccccc3c3cc4oc5ccccc5c4cc32)cc(-n2c3ccccc3c3ccccc32)c1 ; N#Cc1cc(-c2ccccc2-n2c3ccccc3c3c4c(ccc32)oc2ccccc24)cc(-n2c3ccccc3c3ccccc32)c1 ; N#Cc1cc(-c2ccccc2-n2c3ccccc3c3c4oc5ccccc5c4ccc32)cc(-n2c3ccccc3c3ccccc32)c1]

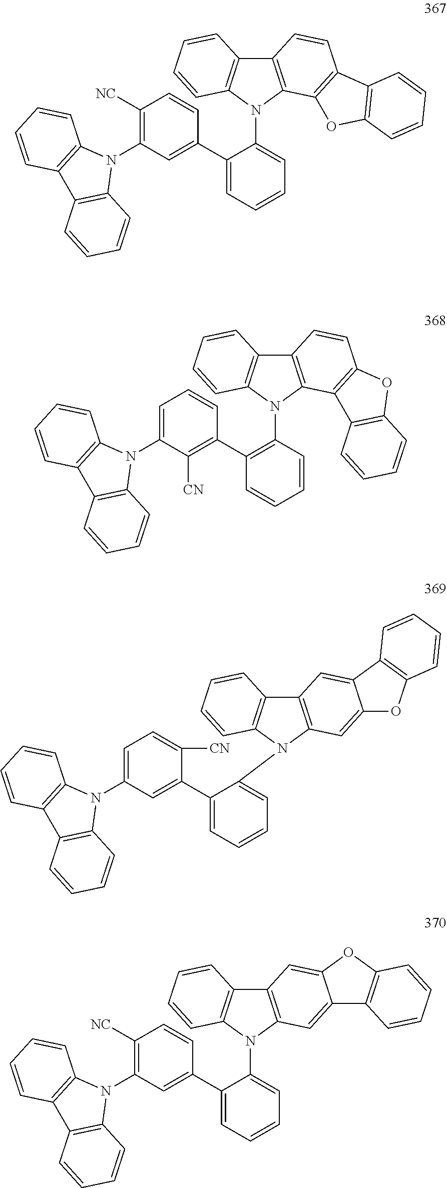 [SMILES: N#Cc1ccc(-c2ccccc2-n2c3ccccc3c3ccc4c5ccccc5oc4c32)cc1-n1c2ccccc2c2ccccc21 ; N#Cc1c(-c2ccccc2-n2c3ccccc3c3ccc4oc5ccccc5c4c32)cccc1-n1c2ccccc2c2ccccc21 ; N#Cc1ccc(-n2c3ccccc3c3ccccc32)cc1-c1ccccc1-n1c2ccccc2c2cc3c(cc21)oc1ccccc13 ; N#Cc1ccc(-c2ccccc2-n2c3ccccc3c3cc4oc5ccccc5c4cc32)cc1-n1c2ccccc2c2ccccc21]

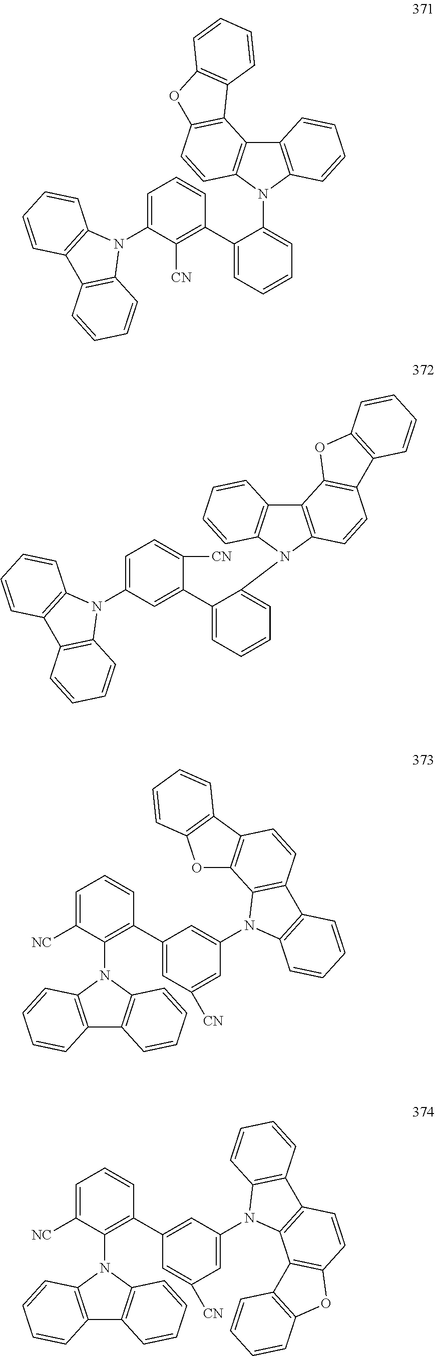 [SMILES: N#Cc1c(-c2ccccc2-n2c3ccccc3c3c4c(ccc32)oc2ccccc24)cccc1-n1c2ccccc2c2ccccc21 ; N#Cc1ccc(-n2c3ccccc3c3ccccc32)cc1-c1ccccc1-n1c2ccccc2c2c3oc4ccccc4c3ccc21 ; N#Cc1cc(-c2cccc(C#N)c2-n2c3ccccc3c3ccccc32)cc(-n2c3ccccc3c3ccc4c5ccccc5oc4c32)c1 ; N#Cc1cc(-c2cccc(C#N)c2-n2c3ccccc3c3ccccc32)cc(-n2c3ccccc3c3ccc4oc5ccccc5c4c32)c1]

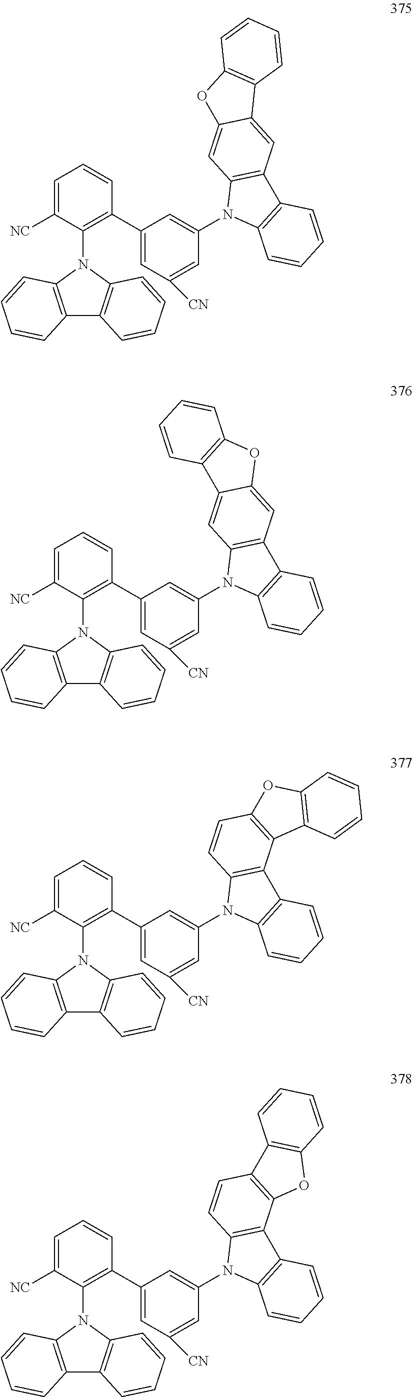 [SMILES: N#Cc1cc(-c2cccc(C#N)c2-n2c3ccccc3c3ccccc32)cc(-n2c3ccccc3c3cc4c(cc32)oc2ccccc24)c1 ; N#Cc1cc(-c2cccc(C#N)c2-n2c3ccccc3c3ccccc32)cc(-n2c3ccccc3c3cc4oc5ccccc5c4cc32)c1 ; N#Cc1cc(-c2cccc(C#N)c2-n2c3ccccc3c3ccccc32)cc(-n2c3ccccc3c3c4c(ccc32)oc2ccccc24)c1 ; N#Cc1cc(-c2cccc(C#N)c2-n2c3ccccc3c3ccccc32)cc(-n2c3ccccc3c3c4oc5ccccc5c4ccc32)c1]

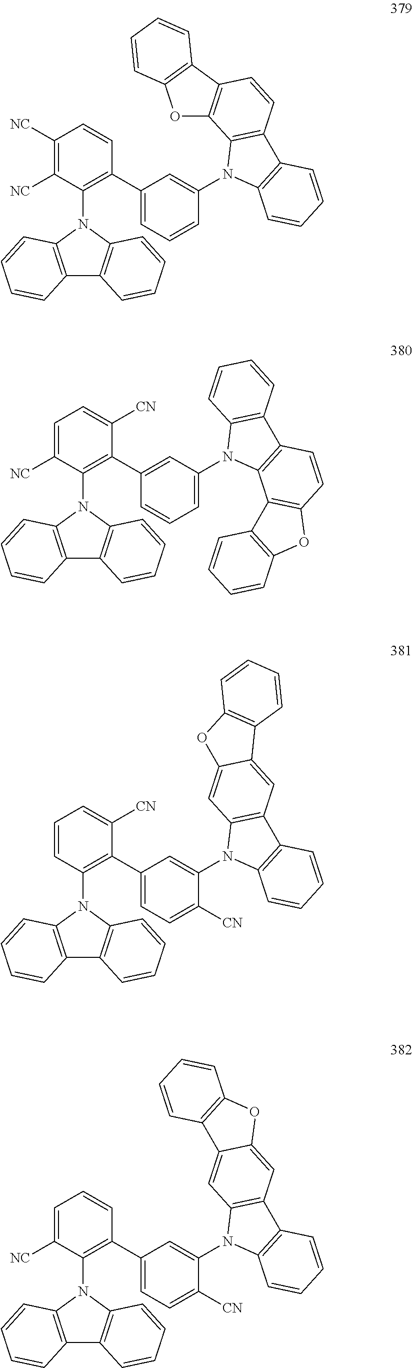 [SMILES: N#Cc1ccc(-c2cccc(-n3c4ccccc4c4ccc5c6ccccc6oc5c43)c2)c(-n2c3ccccc3c3ccccc32)c1C#N ; N#Cc1ccc(C#N)c(-n2c3ccccc3c3ccccc32)c1-c1cccc(-n2c3ccccc3c3ccc4oc5ccccc5c4c32)c1 ; N#Cc1ccc(-c2c(C#N)cccc2-n2c3ccccc3c3ccccc32)cc1-n1c2ccccc2c2cc3c(cc21)oc1ccccc13 ; N#Cc1ccc(-c2cccc(C#N)c2-n2c3ccccc3c3ccccc32)cc1-n1c2ccccc2c2cc3oc4ccccc4c3cc21]

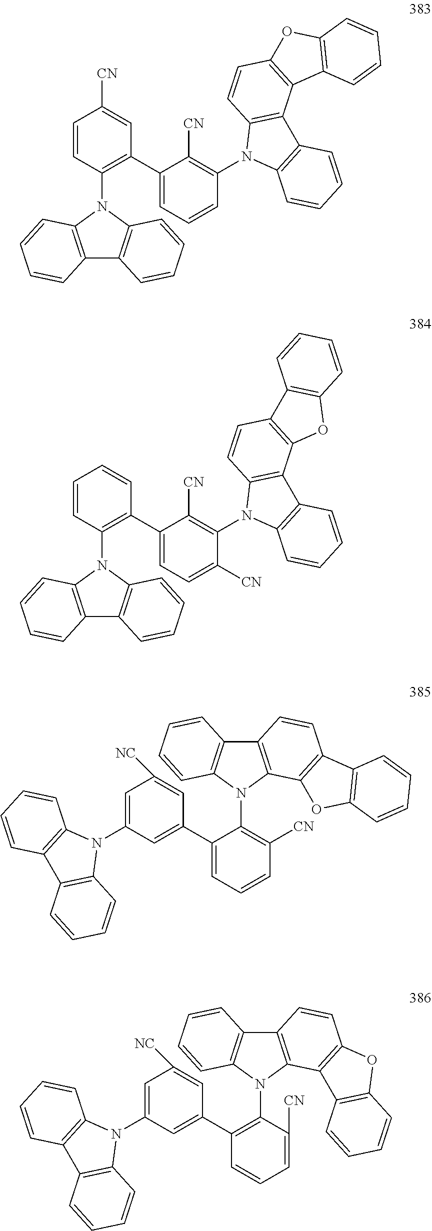 [SMILES: N#Cc1ccc(-n2c3ccccc3c3ccccc32)c(-c2cccc(-n3c4ccccc4c4c5c(ccc43)oc3ccccc35)c2C#N)c1 ; N#Cc1ccc(-c2ccccc2-n2c3ccccc3c3ccccc32)c(C#N)c1-n1c2ccccc2c2c3oc4ccccc4c3ccc21 ; N#Cc1cc(-c2cccc(C#N)c2-n2c3ccccc3c3ccc4c5ccccc5oc4c32)cc(-n2c3ccccc3c3ccccc32)c1 ; N#Cc1cc(-c2cccc(C#N)c2-n2c3ccccc3c3ccc4oc5ccccc5c4c32)cc(-n2c3ccccc3c3ccccc32)c1]

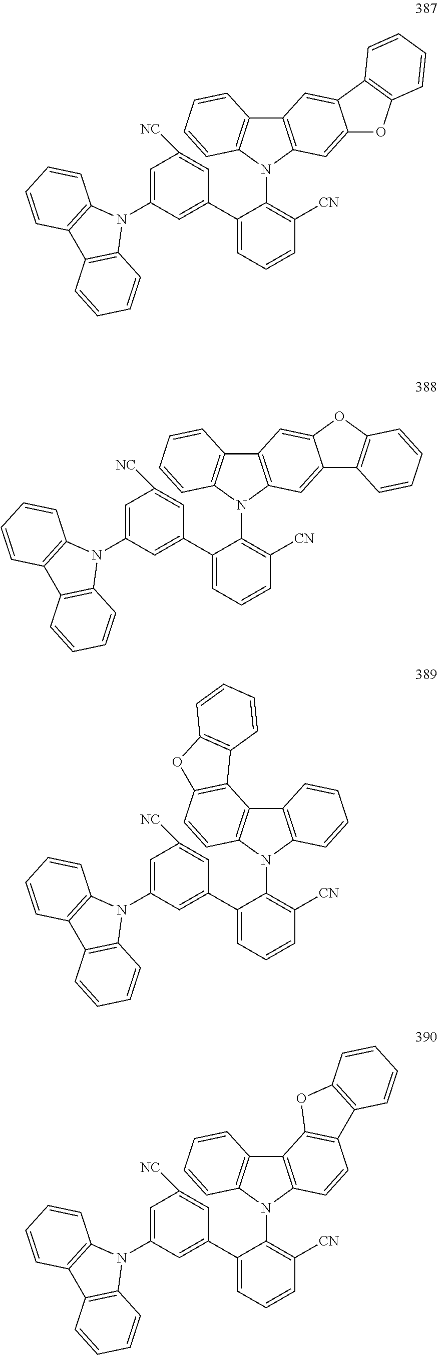 [SMILES: N#Cc1cc(-c2cccc(C#N)c2-n2c3ccccc3c3cc4c(cc32)oc2ccccc24)cc(-n2c3ccccc3c3ccccc32)c1 ; N#Cc1cc(-c2cccc(C#N)c2-n2c3ccccc3c3cc4oc5ccccc5c4cc32)cc(-n2c3ccccc3c3ccccc32)c1 ; N#Cc1cc(-c2cccc(C#N)c2-n2c3ccccc3c3c4c(ccc32)oc2ccccc24)cc(-n2c3ccccc3c3ccccc32)c1 ; N#Cc1cc(-c2cccc(C#N)c2-n2c3ccccc3c3c4oc5ccccc5c4ccc32)cc(-n2c3ccccc3c3ccccc32)c1]

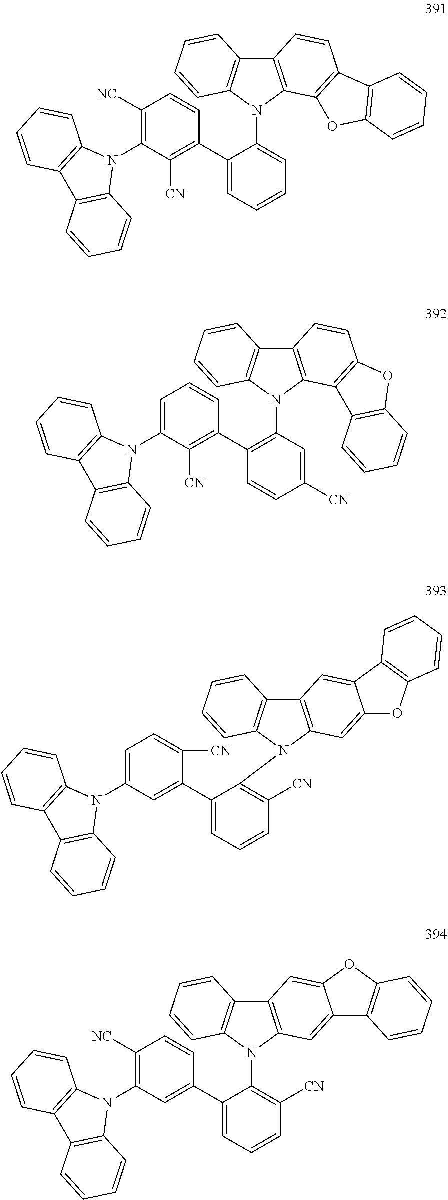 [SMILES: N#Cc1ccc(-c2ccccc2-n2c3ccccc3c3ccc4c5ccccc5oc4c32)c(C#N)c1-n1c2ccccc2c2ccccc21 ; N#Cc1ccc(-c2cccc(-n3c4ccccc4c4ccccc43)c2C#N)c(-n2c3ccccc3c3ccc4oc5ccccc5c4c32)c1 ; N#Cc1ccc(-n2c3ccccc3c3ccccc32)cc1-c1cccc(C#N)c1-n1c2ccccc2c2cc3c(cc21)oc1ccccc13 ; N#Cc1ccc(-c2cccc(C#N)c2-n2c3ccccc3c3cc4oc5ccccc5c4cc32)cc1-n1c2ccccc2c2ccccc21]

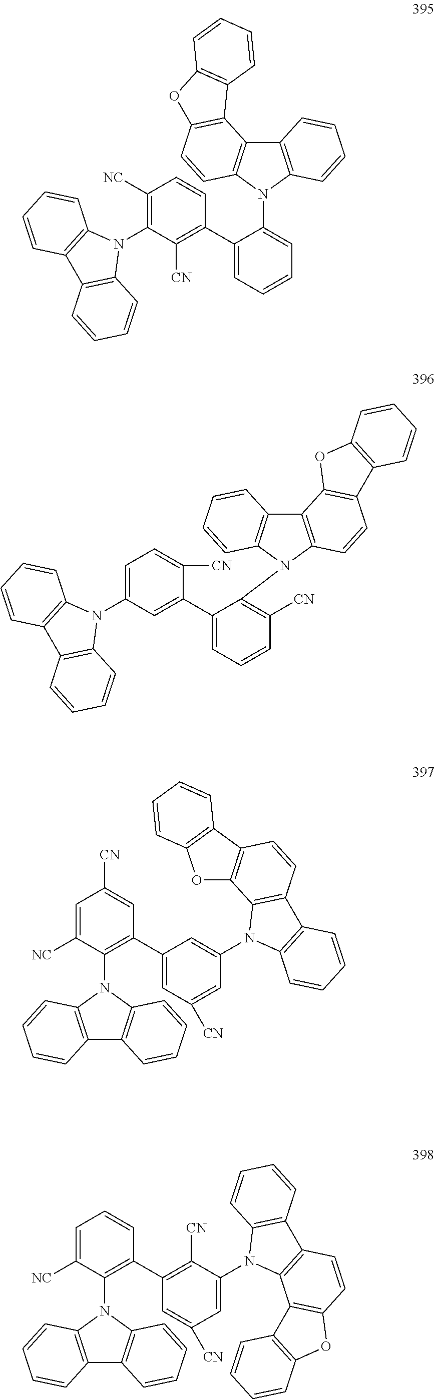 [SMILES: N#Cc1ccc(-c2ccccc2-n2c3ccccc3c3c4c(ccc32)oc2ccccc24)c(C#N)c1-n1c2ccccc2c2ccccc21 ; N#Cc1ccc(-n2c3ccccc3c3ccccc32)cc1-c1cccc(C#N)c1-n1c2ccccc2c2c3oc4ccccc4c3ccc21 ; N#Cc1cc(-c2cc(C#N)cc(C#N)c2-n2c3ccccc3c3ccccc32)cc(-n2c3ccccc3c3ccc4c5ccccc5oc4c32)c1 ; N#Cc1cc(-c2cccc(C#N)c2-n2c3ccccc3c3ccccc32)c(C#N)c(-n2c3ccccc3c3ccc4oc5ccccc5c4c32)c1]

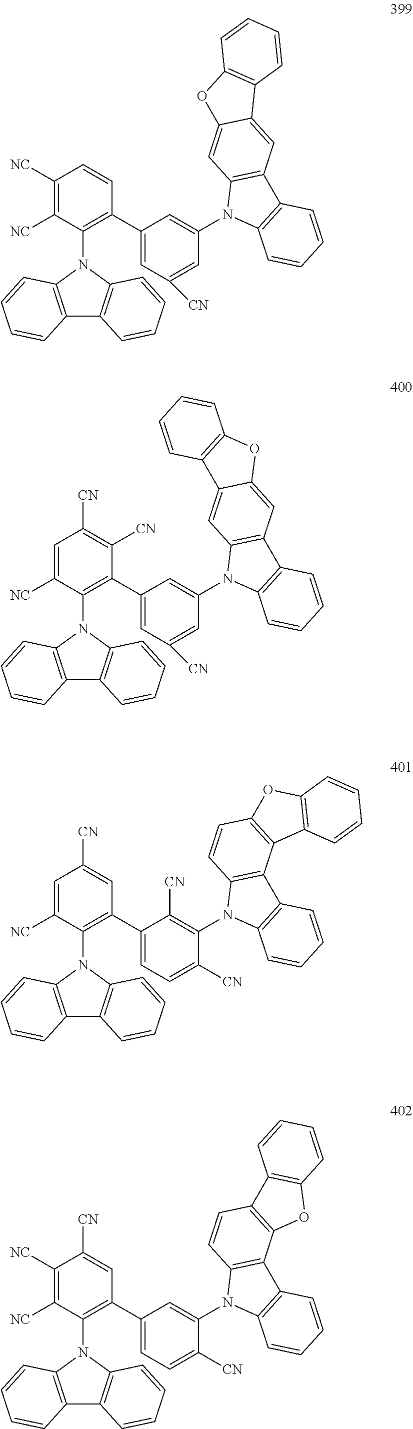 [SMILES: N#Cc1cc(-c2ccc(C#N)c(C#N)c2-n2c3ccccc3c3ccccc32)cc(-n2c3ccccc3c3cc4c(cc32)oc2ccccc24)c1 ; N#Cc1cc(-c2c(C#N)c(C#N)cc(C#N)c2-n2c3ccccc3c3ccccc32)cc(-n2c3ccccc3c3cc4oc5ccccc5c4cc32)c1 ; N#Cc1cc(C#N)c(-n2c3ccccc3c3ccccc32)c(-c2ccc(C#N)c(-n3c4ccccc4c4c5c(ccc43)oc3ccccc35)c2C#N)c1 ; N#Cc1ccc(-c2cc(C#N)c(C#N)c(C#N)c2-n2c3ccccc3c3ccccc32)cc1-n1c2ccccc2c2c3oc4ccccc4c3ccc21]

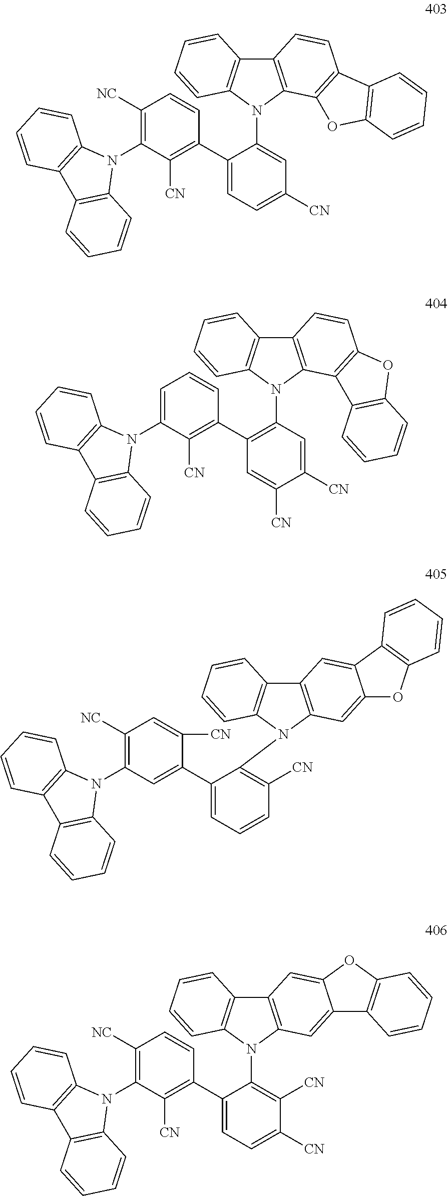 [SMILES: N#Cc1ccc(-c2ccc(C#N)c(-n3c4ccccc4c4ccccc43)c2C#N)c(-n2c3ccccc3c3ccc4c5ccccc5oc4c32)c1 ; N#Cc1cc(-c2cccc(-n3c4ccccc4c4ccccc43)c2C#N)c(-n2c3ccccc3c3ccc4oc5ccccc5c4c32)cc1C#N ; N#Cc1cc(C#N)c(-n2c3ccccc3c3ccccc32)cc1-c1cccc(C#N)c1-n1c2ccccc2c2cc3c(cc21)oc1ccccc13 ; N#Cc1ccc(-c2ccc(C#N)c(-n3c4ccccc4c4ccccc43)c2C#N)c(-n2c3ccccc3c3cc4oc5ccccc5c4cc32)c1C#N]

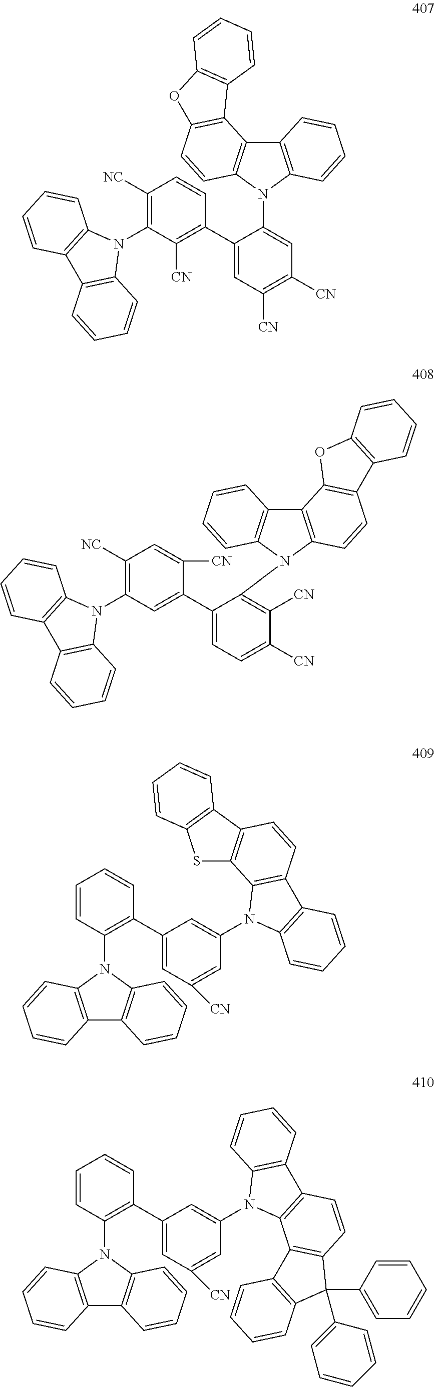 [SMILES: N#Cc1cc(-c2ccc(C#N)c(-n3c4ccccc4c4ccccc43)c2C#N)c(-n2c3ccccc3c3c4c(ccc32)oc2ccccc24)cc1C#N ; N#Cc1cc(C#N)c(-n2c3ccccc3c3ccccc32)cc1-c1ccc(C#N)c(C#N)c1-n1c2ccccc2c2c3oc4ccccc4c3ccc21 ; N#Cc1cc(-c2ccccc2-n2c3ccccc3c3ccccc32)cc(-n2c3ccccc3c3ccc4c5ccccc5sc4c32)c1 ; N#Cc1cc(-c2ccccc2-n2c3ccccc3c3ccccc32)cc(-n2c3ccccc3c3ccc4c(c32)-c2ccccc2C4(c2ccccc2)c2ccccc2)c1]

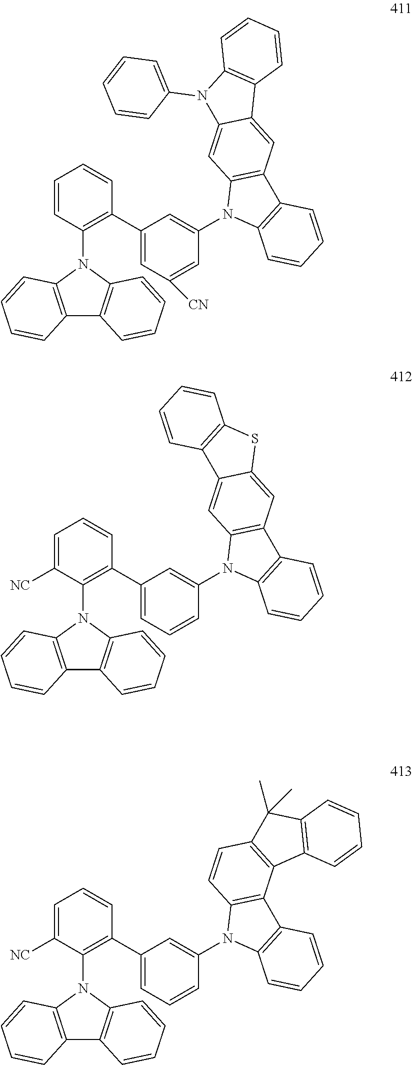 [SMILES: N#Cc1cc(-c2ccccc2-n2c3ccccc3c3ccccc32)cc(-n2c3ccccc3c3cc4c5ccccc5n(-c5ccccc5)c4cc32)c1 ; N#Cc1cccc(-c2cccc(-n3c4ccccc4c4cc5sc6ccccc6c5cc43)c2)c1-n1c2ccccc2c2ccccc21 ; CC1(C)c2ccccc2-c2c1ccc1c2c2ccccc2n1-c1cccc(-c2cccc(C#N)c2-n2c3ccccc3c3ccccc32)c1]

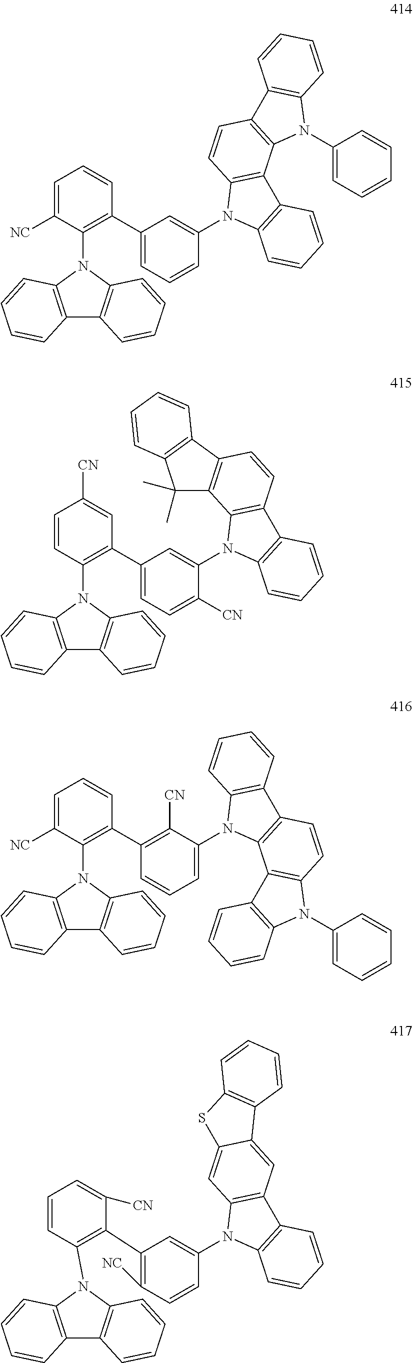 [SMILES: N#Cc1cccc(-c2cccc(-n3c4ccccc4c4c3ccc3c5ccccc5n(-c5ccccc5)c34)c2)c1-n1c2ccccc2c2ccccc21 ; CC1(C)c2ccccc2-c2ccc3c4ccccc4n(-c4cc(-c5cc(C#N)ccc5-n5c6ccccc6c6ccccc65)ccc4C#N)c3c21 ; N#Cc1cccc(-c2cccc(-n3c4ccccc4c4ccc5c(c6ccccc6n5-c5ccccc5)c43)c2C#N)c1-n1c2ccccc2c2ccccc21 ; N#Cc1ccc(-n2c3ccccc3c3cc4c(cc32)sc2ccccc24)cc1-c1c(C#N)cccc1-n1c2ccccc2c2ccccc21]

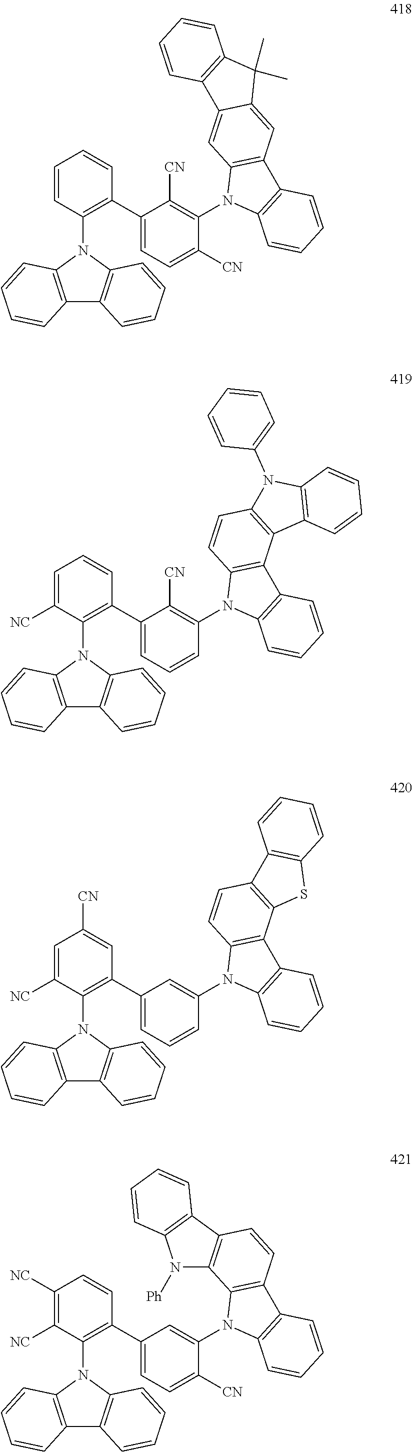 [SMILES: CC1(C)c2ccccc2-c2cc3c(cc21)c1ccccc1n3-c1c(C#N)ccc(-c2ccccc2-n2c3ccccc3c3ccccc32)c1C#N ; N#Cc1cccc(-c2cccc(-n3c4ccccc4c4c5c6ccccc6n(-c6ccccc6)c5ccc43)c2C#N)c1-n1c2ccccc2c2ccccc21 ; N#Cc1cc(C#N)c(-n2c3ccccc3c3ccccc32)c(-c2cccc(-n3c4ccccc4c4c5sc6ccccc6c5ccc43)c2)c1 ; N#Cc1ccc(-c2ccc(C#N)c(C#N)c2-n2c3ccccc3c3ccccc32)cc1-n1c2ccccc2c2ccc3c4ccccc4n(-c4ccccc4)c3c21]

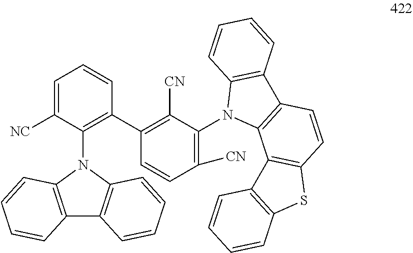 [SMILES: N#Cc1cccc(-c2ccc(C#N)c(-n3c4ccccc4c4ccc5sc6ccccc6c5c43)c2C#N)c1-n1c2ccccc2c2ccccc21]

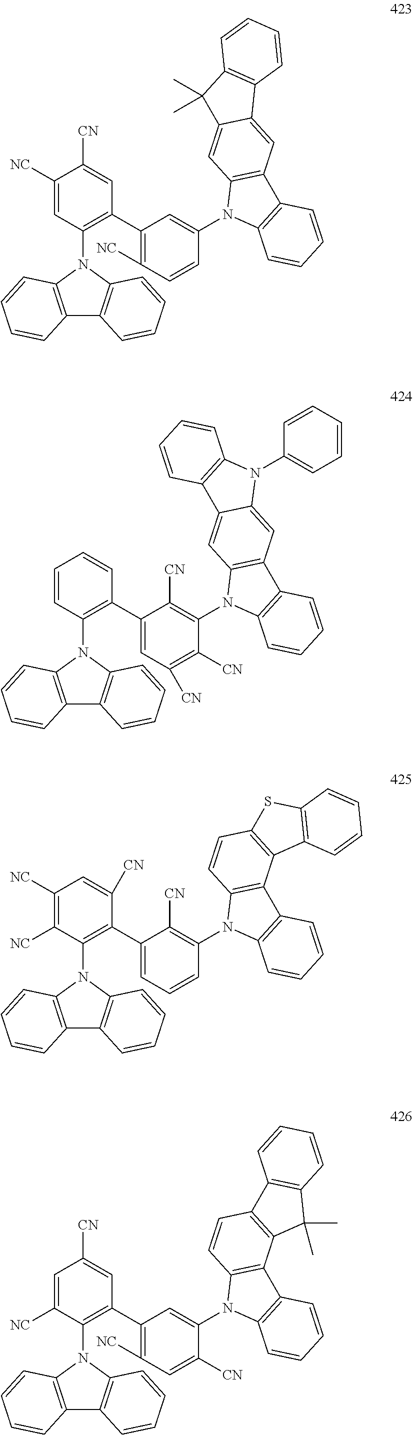 [SMILES: CC1(C)c2ccccc2-c2cc3c4ccccc4n(-c4ccc(C#N)c(-c5cc(C#N)c(C#N)cc5-n5c6ccccc6c6ccccc65)c4)c3cc21 ; N#Cc1cc(-c2ccccc2-n2c3ccccc3c3ccccc32)c(C#N)c(-n2c3ccccc3c3cc4c(cc32)c2ccccc2n4-c2ccccc2)c1C#N ; N#Cc1cc(C#N)c(-c2cccc(-n3c4ccccc4c4c5c(ccc43)sc3ccccc35)c2C#N)c(-n2c3ccccc3c3ccccc32)c1C#N ; CC1(C)c2ccccc2-c2ccc3c(c21)c1ccccc1n3-c1cc(-c2cc(C#N)cc(C#N)c2-n2c3ccccc3c3ccccc32)c(C#N)cc1C#N]

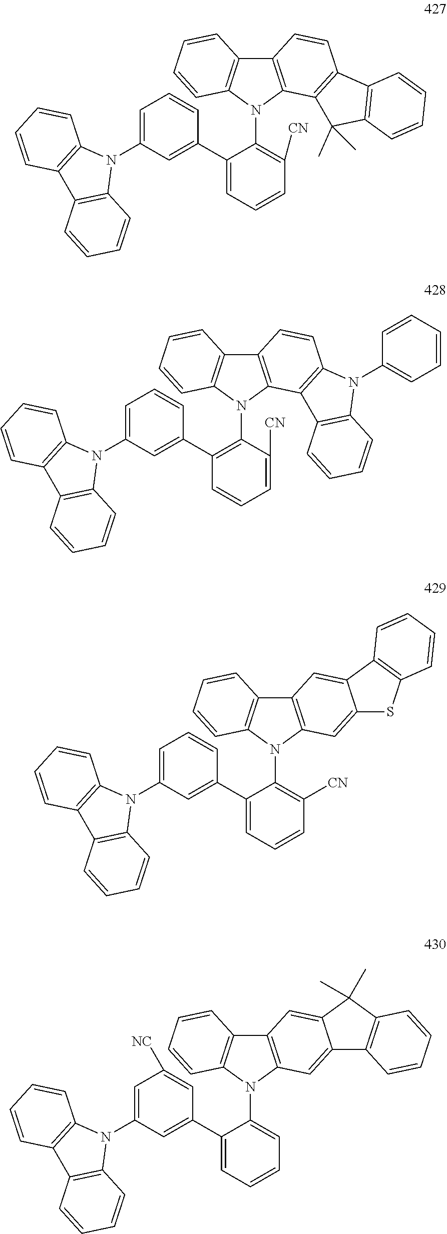 [SMILES: CC1(C)c2ccccc2-c2ccc3c4ccccc4n(-c4c(C#N)cccc4-c4cccc(-n5c6ccccc6c6ccccc65)c4)c3c21 ; N#Cc1cccc(-c2cccc(-n3c4ccccc4c4ccccc43)c2)c1-n1c2ccccc2c2ccc3c(c4ccccc4n3-c3ccccc3)c21 ; N#Cc1cccc(-c2cccc(-n3c4ccccc4c4ccccc43)c2)c1-n1c2ccccc2c2cc3c(cc21)sc1ccccc13 ; CC1(C)c2ccccc2-c2cc3c(cc21)c1ccccc1n3-c1ccccc1-c1cc(C#N)cc(-n2c3ccccc3c3ccccc32)c1]

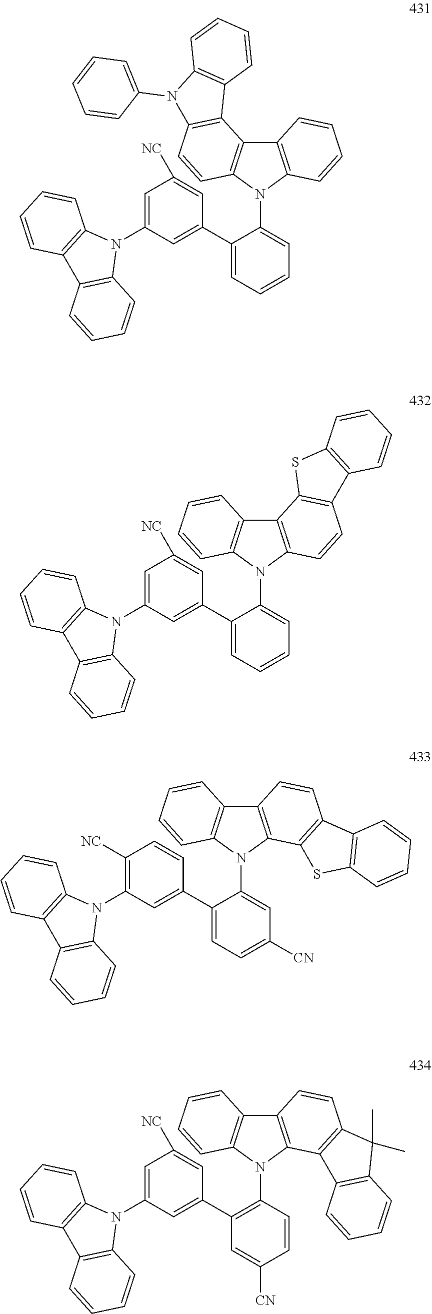 [SMILES: N#Cc1cc(-c2ccccc2-n2c3ccccc3c3c4c5ccccc5n(-c5ccccc5)c4ccc32)cc(-n2c3ccccc3c3ccccc32)c1 ; N#Cc1cc(-c2ccccc2-n2c3ccccc3c3c4sc5ccccc5c4ccc32)cc(-n2c3ccccc3c3ccccc32)c1 ; N#Cc1ccc(-c2ccc(C#N)c(-n3c4ccccc4c4ccccc43)c2)c(-n2c3ccccc3c3ccc4c5ccccc5sc4c32)c1 ; CC1(C)c2ccccc2-c2c1ccc1c3ccccc3n(-c3ccc(C#N)cc3-c3cc(C#N)cc(-n4c5ccccc5c5ccccc54)c3)c21]

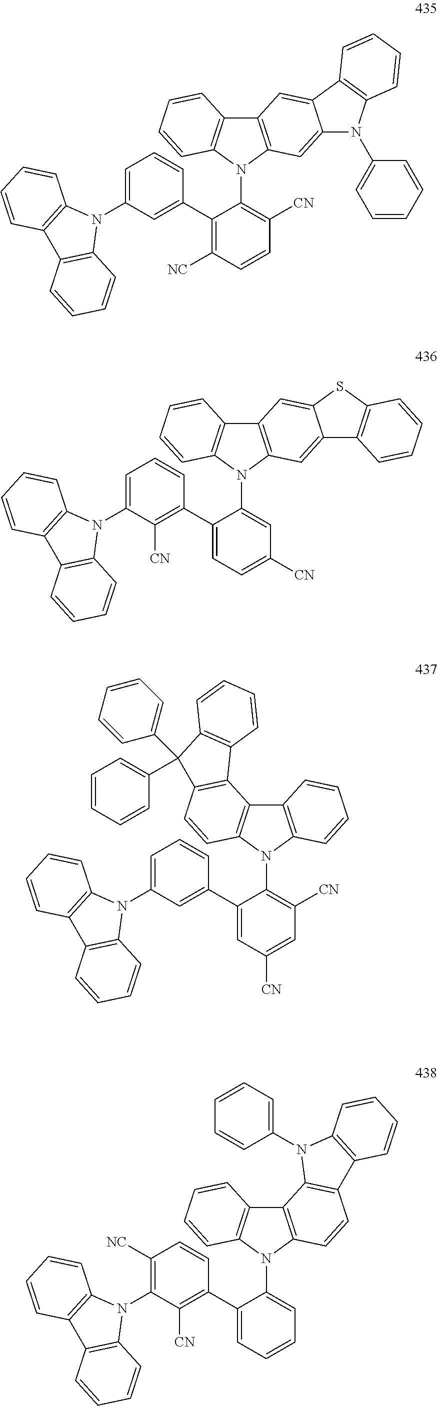 [SMILES: N#Cc1ccc(C#N)c(-n2c3ccccc3c3cc4c5ccccc5n(-c5ccccc5)c4cc32)c1-c1cccc(-n2c3ccccc3c3ccccc32)c1 ; N#Cc1ccc(-c2cccc(-n3c4ccccc4c4ccccc43)c2C#N)c(-n2c3ccccc3c3cc4sc5ccccc5c4cc32)c1 ; N#Cc1cc(C#N)c(-n2c3ccccc3c3c4c(ccc32)C(c2ccccc2)(c2ccccc2)c2ccccc2-4)c(-c2cccc(-n3c4ccccc4c4ccccc43)c2)c1 ; N#Cc1ccc(-c2ccccc2-n2c3ccccc3c3c2ccc2c4ccccc4n(-c4ccccc4)c23)c(C#N)c1-n1c2ccccc2c2ccccc21]

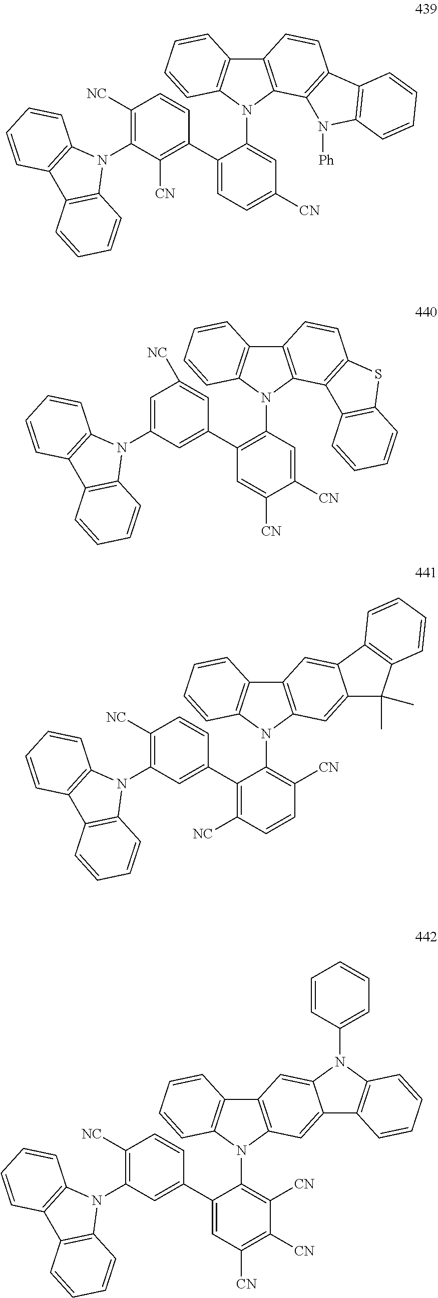 [SMILES: N#Cc1ccc(-c2ccc(C#N)c(-n3c4ccccc4c4ccccc43)c2C#N)c(-n2c3ccccc3c3ccc4c5ccccc5n(-c5ccccc5)c4c32)c1 ; N#Cc1cc(-c2cc(C#N)c(C#N)cc2-n2c3ccccc3c3ccc4sc5ccccc5c4c32)cc(-n2c3ccccc3c3ccccc32)c1 ; CC1(C)c2ccccc2-c2cc3c4ccccc4n(-c4c(C#N)ccc(C#N)c4-c4ccc(C#N)c(-n5c6ccccc6c6ccccc65)c4)c3cc21 ; N#Cc1ccc(-c2cc(C#N)c(C#N)c(C#N)c2-n2c3ccccc3c3cc4c(cc32)c2ccccc2n4-c2ccccc2)cc1-n1c2ccccc2c2ccccc21]

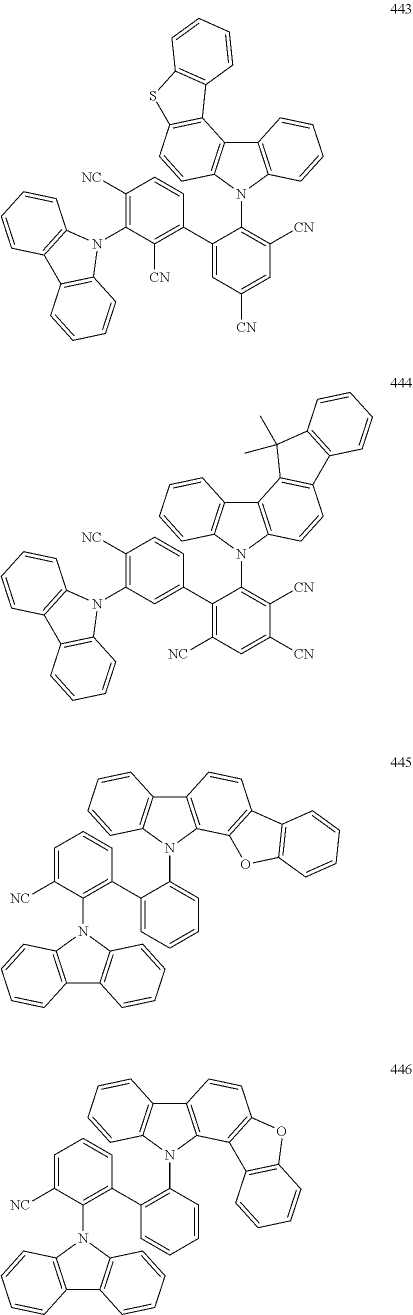 [SMILES: N#Cc1cc(C#N)c(-n2c3ccccc3c3c4c(ccc32)sc2ccccc24)c(-c2ccc(C#N)c(-n3c4ccccc4c4ccccc43)c2C#N)c1 ; CC1(C)c2ccccc2-c2ccc3c(c21)c1ccccc1n3-c1c(C#N)c(C#N)cc(C#N)c1-c1ccc(C#N)c(-n2c3ccccc3c3ccccc32)c1 ; N#Cc1cccc(-c2ccccc2-n2c3ccccc3c3ccc4c5ccccc5oc4c32)c1-n1c2ccccc2c2ccccc21 ; N#Cc1cccc(-c2ccccc2-n2c3ccccc3c3ccc4oc5ccccc5c4c32)c1-n1c2ccccc2c2ccccc21]

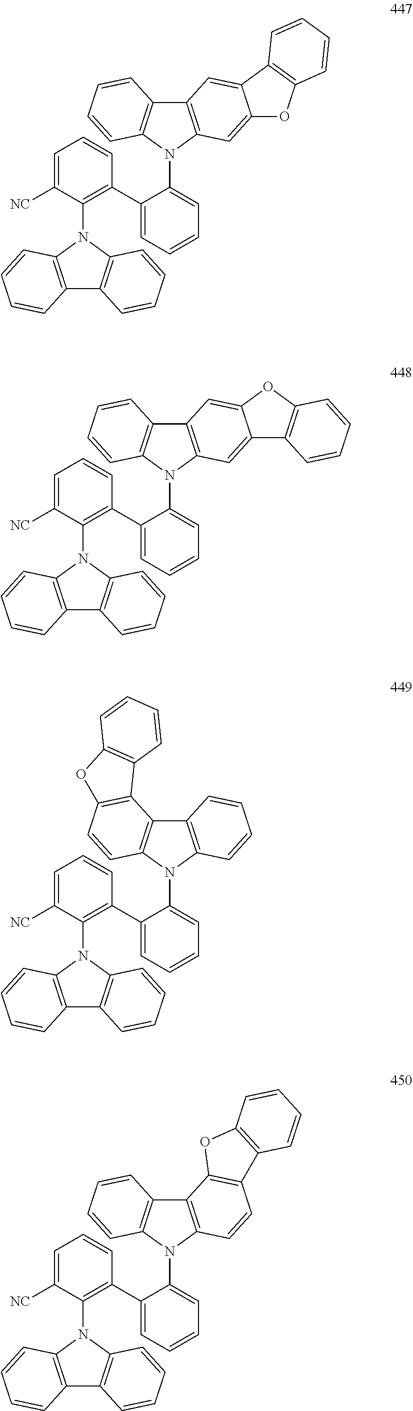 [SMILES: N#Cc1cccc(-c2ccccc2-n2c3ccccc3c3cc4c(cc32)oc2ccccc24)c1-n1c2ccccc2c2ccccc21 ; N#Cc1cccc(-c2ccccc2-n2c3ccccc3c3cc4oc5ccccc5c4cc32)c1-n1c2ccccc2c2ccccc21 ; N#Cc1cccc(-c2ccccc2-n2c3ccccc3c3c4c(ccc32)oc2ccccc24)c1-n1c2ccccc2c2ccccc21 ; N#Cc1cccc(-c2ccccc2-n2c3ccccc3c3c4oc5ccccc5c4ccc32)c1-n1c2ccccc2c2ccccc21]

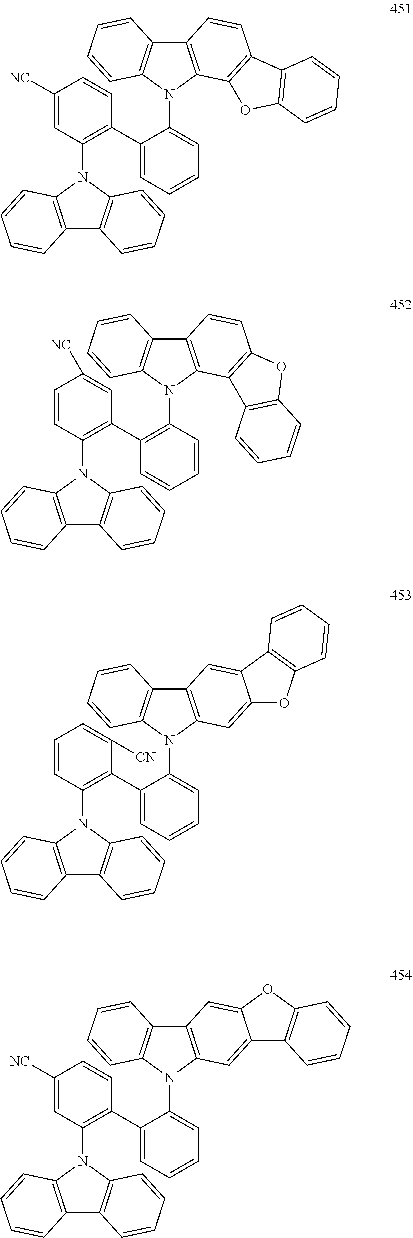 [SMILES: N#Cc1ccc(-c2ccccc2-n2c3ccccc3c3ccc4c5ccccc5oc4c32)c(-n2c3ccccc3c3ccccc32)c1 ; N#Cc1ccc(-n2c3ccccc3c3ccccc32)c(-c2ccccc2-n2c3ccccc3c3ccc4oc5ccccc5c4c32)c1 ; N#Cc1cccc(-n2c3ccccc3c3ccccc32)c1-c1ccccc1-n1c2ccccc2c2cc3c(cc21)oc1ccccc13 ; N#Cc1ccc(-c2ccccc2-n2c3ccccc3c3cc4oc5ccccc5c4cc32)c(-n2c3ccccc3c3ccccc32)c1]

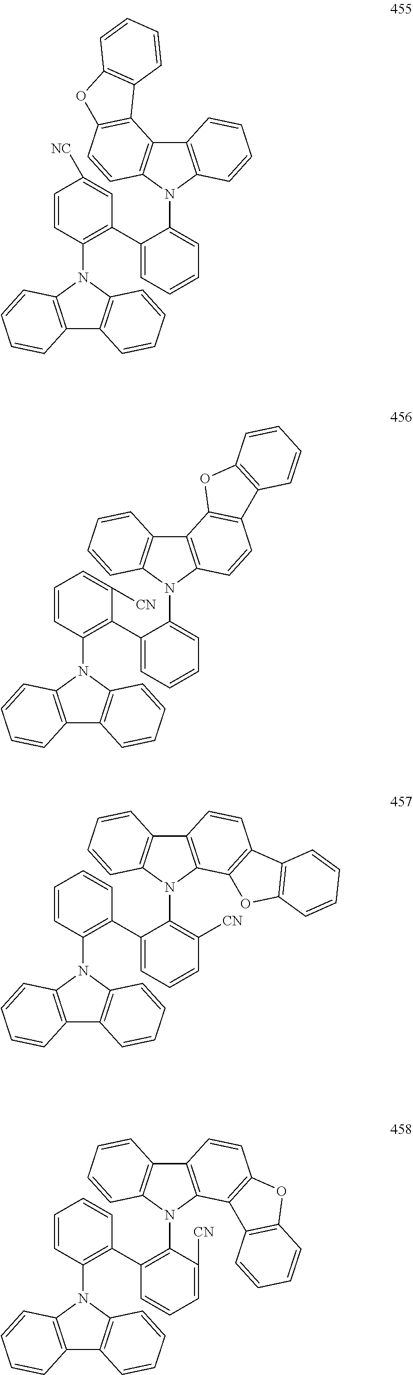 [SMILES: N#Cc1ccc(-n2c3ccccc3c3ccccc32)c(-c2ccccc2-n2c3ccccc3c3c4c(ccc32)oc2ccccc24)c1 ; N#Cc1cccc(-n2c3ccccc3c3ccccc32)c1-c1ccccc1-n1c2ccccc2c2c3oc4ccccc4c3ccc21 ; N#Cc1cccc(-c2ccccc2-n2c3ccccc3c3ccccc32)c1-n1c2ccccc2c2ccc3c4ccccc4oc3c21 ; N#Cc1cccc(-c2ccccc2-n2c3ccccc3c3ccccc32)c1-n1c2ccccc2c2ccc3oc4ccccc4c3c21]

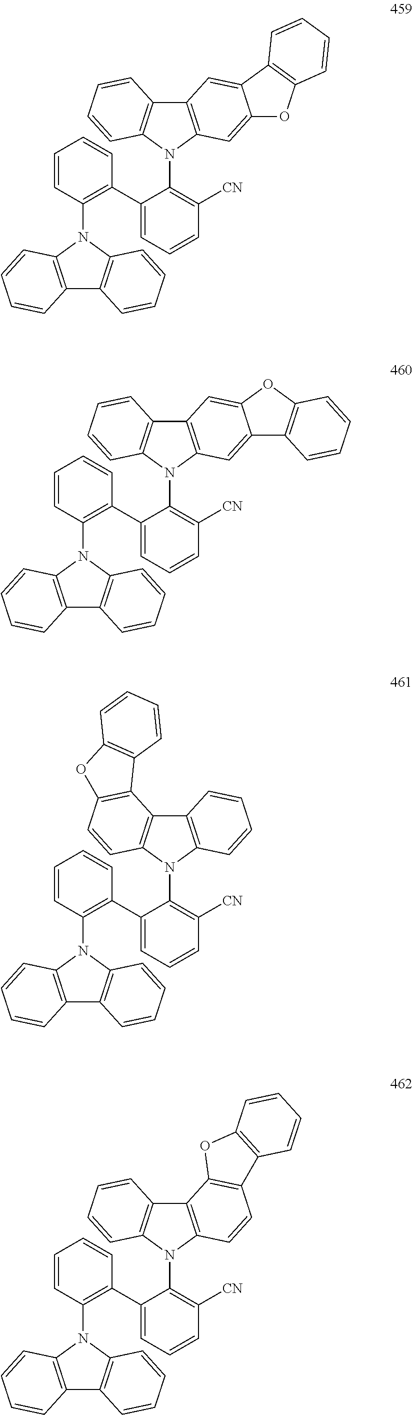 [SMILES: N#Cc1cccc(-c2ccccc2-n2c3ccccc3c3ccccc32)c1-n1c2ccccc2c2cc3c(cc21)oc1ccccc13 ; N#Cc1cccc(-c2ccccc2-n2c3ccccc3c3ccccc32)c1-n1c2ccccc2c2cc3oc4ccccc4c3cc21 ; N#Cc1cccc(-c2ccccc2-n2c3ccccc3c3ccccc32)c1-n1c2ccccc2c2c3c(ccc21)oc1ccccc13 ; N#Cc1cccc(-c2ccccc2-n2c3ccccc3c3ccccc32)c1-n1c2ccccc2c2c3oc4ccccc4c3ccc21]

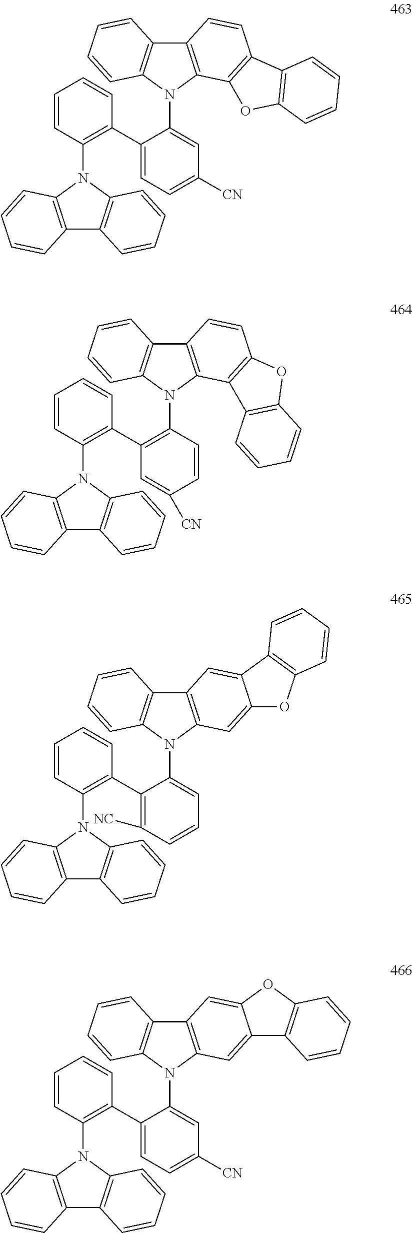 [SMILES: N#Cc1ccc(-c2ccccc2-n2c3ccccc3c3ccccc32)c(-n2c3ccccc3c3ccc4c5ccccc5oc4c32)c1 ; N#Cc1ccc(-n2c3ccccc3c3ccc4oc5ccccc5c4c32)c(-c2ccccc2-n2c3ccccc3c3ccccc32)c1 ; N#Cc1cccc(-n2c3ccccc3c3cc4c(cc32)oc2ccccc24)c1-c1ccccc1-n1c2ccccc2c2ccccc21 ; N#Cc1ccc(-c2ccccc2-n2c3ccccc3c3ccccc32)c(-n2c3ccccc3c3cc4oc5ccccc5c4cc32)c1]

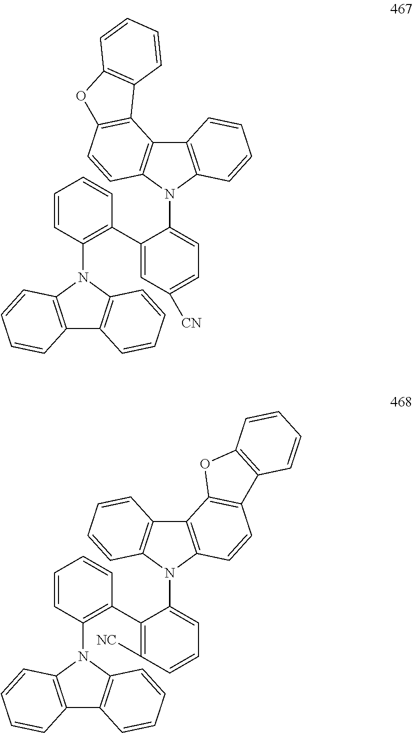 [SMILES: N#Cc1ccc(-n2c3ccccc3c3c4c(ccc32)oc2ccccc24)c(-c2ccccc2-n2c3ccccc3c3ccccc32)c1 ; N#Cc1cccc(-n2c3ccccc3c3c4oc5ccccc5c4ccc32)c1-c1ccccc1-n1c2ccccc2c2ccccc21]

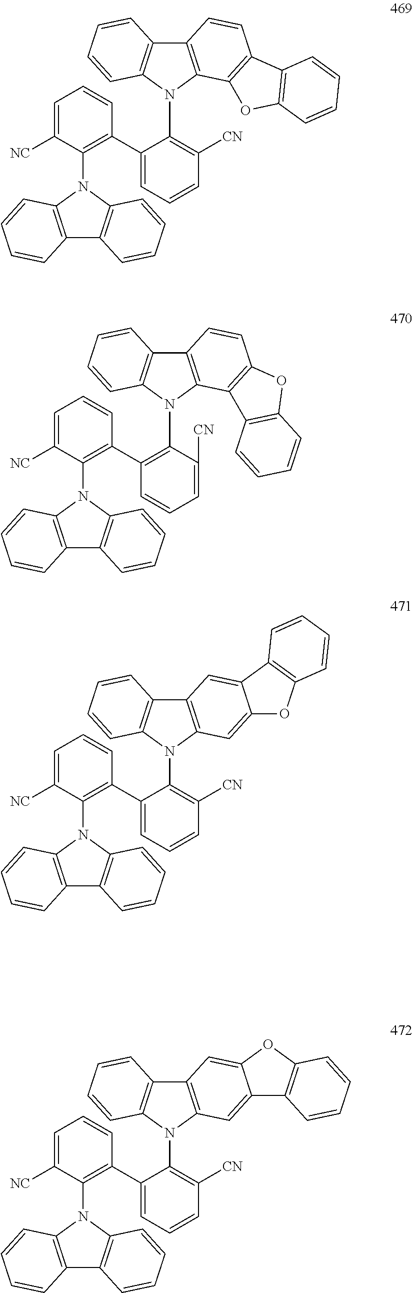 [SMILES: N#Cc1cccc(-c2cccc(C#N)c2-n2c3ccccc3c3ccc4c5ccccc5oc4c32)c1-n1c2ccccc2c2ccccc21 ; N#Cc1cccc(-c2cccc(C#N)c2-n2c3ccccc3c3ccc4oc5ccccc5c4c32)c1-n1c2ccccc2c2ccccc21 ; N#Cc1cccc(-c2cccc(C#N)c2-n2c3ccccc3c3cc4c(cc32)oc2ccccc24)c1-n1c2ccccc2c2ccccc21 ; N#Cc1cccc(-c2cccc(C#N)c2-n2c3ccccc3c3cc4oc5ccccc5c4cc32)c1-n1c2ccccc2c2ccccc21]

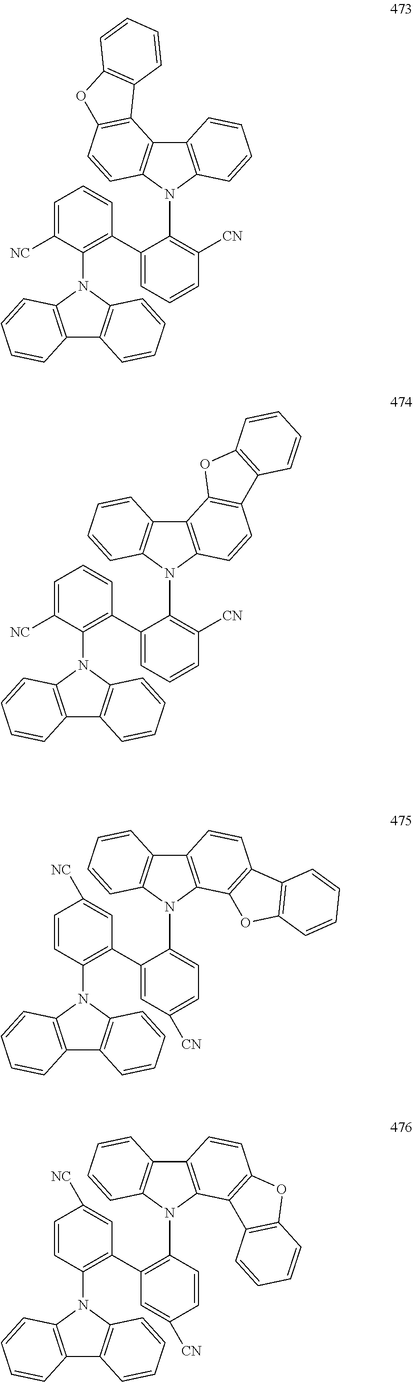 [SMILES: N#Cc1cccc(-c2cccc(C#N)c2-n2c3ccccc3c3c4c(ccc32)oc2ccccc24)c1-n1c2ccccc2c2ccccc21 ; N#Cc1cccc(-c2cccc(C#N)c2-n2c3ccccc3c3c4oc5ccccc5c4ccc32)c1-n1c2ccccc2c2ccccc21 ; N#Cc1ccc(-n2c3ccccc3c3ccccc32)c(-c2cc(C#N)ccc2-n2c3ccccc3c3ccc4c5ccccc5oc4c32)c1 ; N#Cc1ccc(-n2c3ccccc3c3ccccc32)c(-c2cc(C#N)ccc2-n2c3ccccc3c3ccc4oc5ccccc5c4c32)c1]

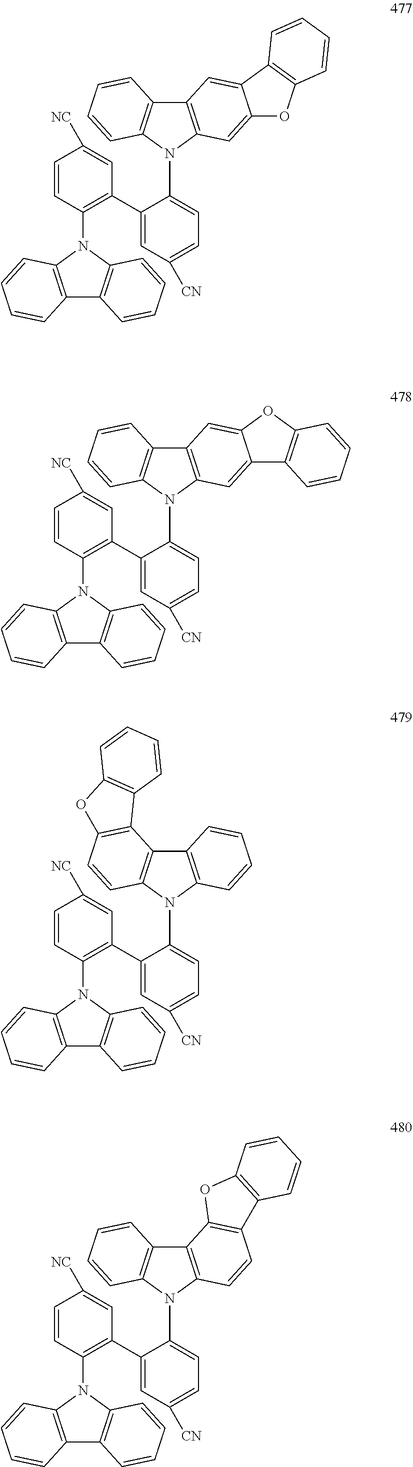 [SMILES: N#Cc1ccc(-n2c3ccccc3c3ccccc32)c(-c2cc(C#N)ccc2-n2c3ccccc3c3cc4c(cc32)oc2ccccc24)c1 ; N#Cc1ccc(-n2c3ccccc3c3ccccc32)c(-c2cc(C#N)ccc2-n2c3ccccc3c3cc4oc5ccccc5c4cc32)c1 ; N#Cc1ccc(-n2c3ccccc3c3ccccc32)c(-c2cc(C#N)ccc2-n2c3ccccc3c3c4c(ccc32)oc2ccccc24)c1 ; N#Cc1ccc(-n2c3ccccc3c3ccccc32)c(-c2cc(C#N)ccc2-n2c3ccccc3c3c4oc5ccccc5c4ccc32)c1]

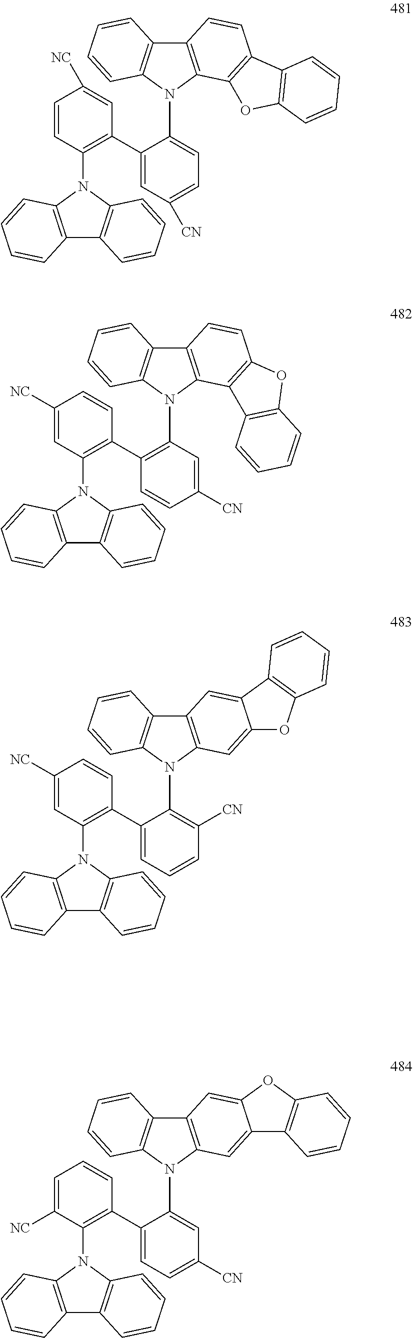 [SMILES: N#Cc1ccc(-n2c3ccccc3c3ccccc32)c(-c2cc(C#N)ccc2-n2c3ccccc3c3ccc4c5ccccc5oc4c32)c1 ; N#Cc1ccc(-c2ccc(C#N)cc2-n2c3ccccc3c3ccc4oc5ccccc5c4c32)c(-n2c3ccccc3c3ccccc32)c1 ; N#Cc1ccc(-c2cccc(C#N)c2-n2c3ccccc3c3cc4c(cc32)oc2ccccc24)c(-n2c3ccccc3c3ccccc32)c1 ; N#Cc1ccc(-c2cccc(C#N)c2-n2c3ccccc3c3ccccc32)c(-n2c3ccccc3c3cc4oc5ccccc5c4cc32)c1]

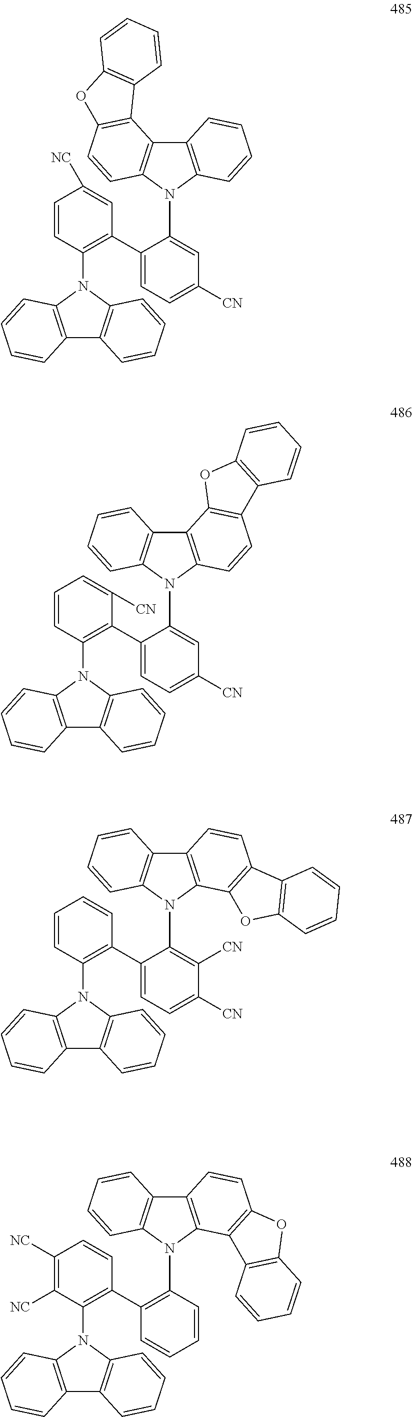 [SMILES: N#Cc1ccc(-n2c3ccccc3c3ccccc32)c(-c2ccc(C#N)cc2-n2c3ccccc3c3c4c(ccc32)oc2ccccc24)c1 ; N#Cc1ccc(-c2c(C#N)cccc2-n2c3ccccc3c3ccccc32)c(-n2c3ccccc3c3c4oc5ccccc5c4ccc32)c1 ; N#Cc1ccc(-c2ccccc2-n2c3ccccc3c3ccccc32)c(-n2c3ccccc3c3ccc4c5ccccc5oc4c32)c1C#N ; N#Cc1ccc(-c2ccccc2-n2c3ccccc3c3ccc4oc5ccccc5c4c32)c(-n2c3ccccc3c3ccccc32)c1C#N]

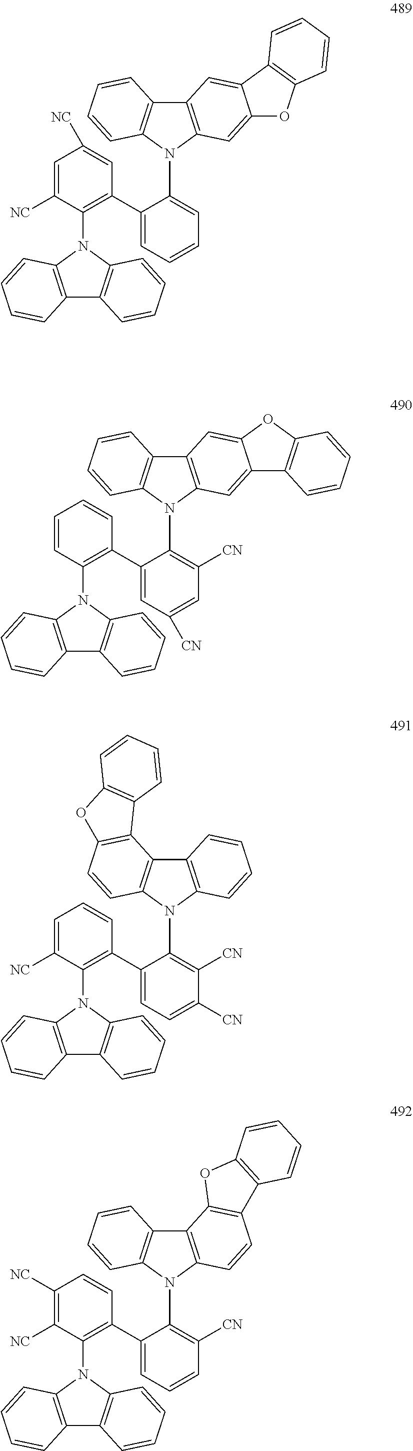 [SMILES: N#Cc1cc(C#N)c(-n2c3ccccc3c3ccccc32)c(-c2ccccc2-n2c3ccccc3c3cc4c(cc32)oc2ccccc24)c1 ; N#Cc1cc(C#N)c(-n2c3ccccc3c3cc4oc5ccccc5c4cc32)c(-c2ccccc2-n2c3ccccc3c3ccccc32)c1 ; N#Cc1ccc(-c2cccc(C#N)c2-n2c3ccccc3c3ccccc32)c(-n2c3ccccc3c3c4c(ccc32)oc2ccccc24)c1C#N ; N#Cc1ccc(-c2cccc(C#N)c2-n2c3ccccc3c3c4oc5ccccc5c4ccc32)c(-n2c3ccccc3c3ccccc32)c1C#N]

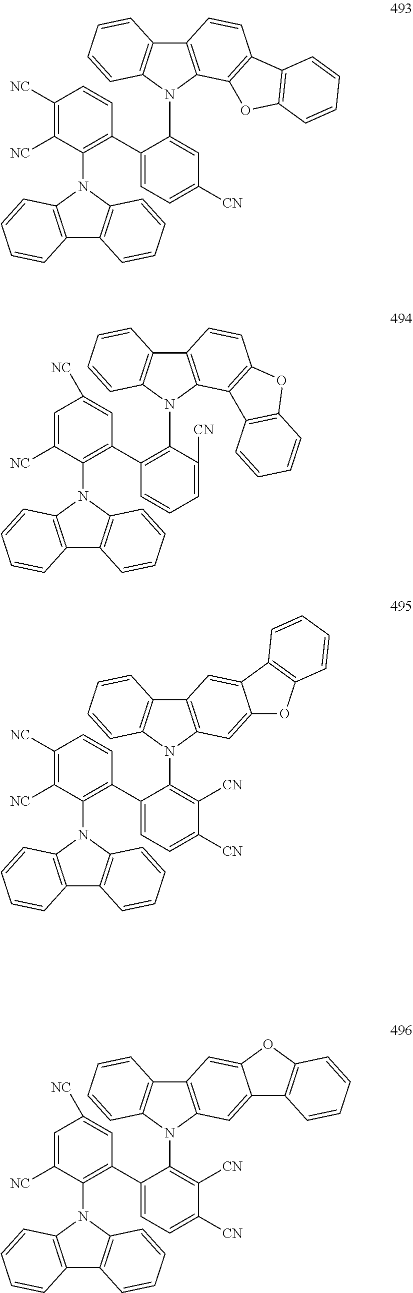 [SMILES: N#Cc1ccc(-c2ccc(C#N)c(C#N)c2-n2c3ccccc3c3ccccc32)c(-n2c3ccccc3c3ccc4c5ccccc5oc4c32)c1 ; N#Cc1cc(C#N)c(-n2c3ccccc3c3ccccc32)c(-c2cccc(C#N)c2-n2c3ccccc3c3ccc4oc5ccccc5c4c32)c1 ; N#Cc1ccc(-c2ccc(C#N)c(C#N)c2-n2c3ccccc3c3cc4c(cc32)oc2ccccc24)c(-n2c3ccccc3c3ccccc32)c1C#N ; N#Cc1cc(C#N)c(-n2c3ccccc3c3ccccc32)c(-c2ccc(C#N)c(C#N)c2-n2c3ccccc3c3cc4oc5ccccc5c4cc32)c1]

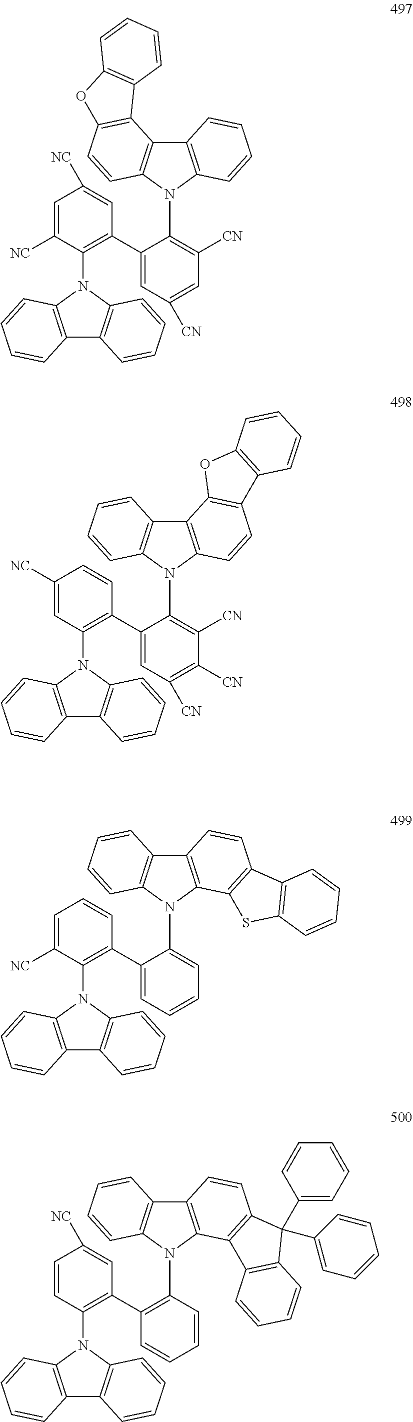 [SMILES: N#Cc1cc(C#N)c(-n2c3ccccc3c3ccccc32)c(-c2cc(C#N)cc(C#N)c2-n2c3ccccc3c3c4c(ccc32)oc2ccccc24)c1 ; N#Cc1ccc(-c2cc(C#N)c(C#N)c(C#N)c2-n2c3ccccc3c3c4oc5ccccc5c4ccc32)c(-n2c3ccccc3c3ccccc32)c1 ; N#Cc1cccc(-c2ccccc2-n2c3ccccc3c3ccc4c5ccccc5sc4c32)c1-n1c2ccccc2c2ccccc21 ; N#Cc1ccc(-n2c3ccccc3c3ccccc32)c(-c2ccccc2-n2c3ccccc3c3ccc4c(c32)-c2ccccc2C4(c2ccccc2)c2ccccc2)c1]

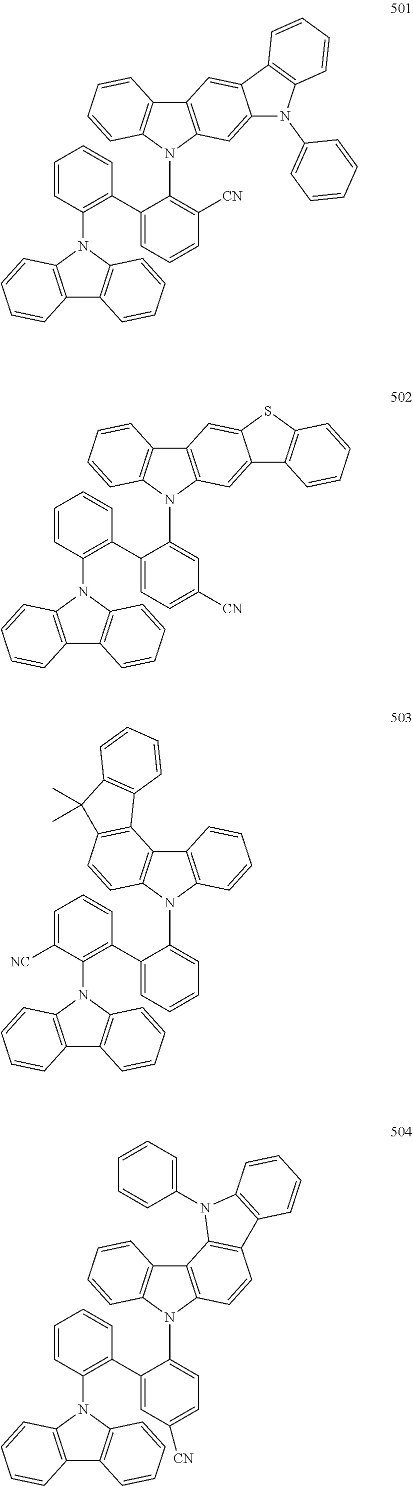 [SMILES: N#Cc1cccc(-c2ccccc2-n2c3ccccc3c3ccccc32)c1-n1c2ccccc2c2cc3c4ccccc4n(-c4ccccc4)c3cc21 ; N#Cc1ccc(-c2ccccc2-n2c3ccccc3c3ccccc32)c(-n2c3ccccc3c3cc4sc5ccccc5c4cc32)c1 ; CC1(C)c2ccccc2-c2c1ccc1c2c2ccccc2n1-c1ccccc1-c1cccc(C#N)c1-n1c2ccccc2c2ccccc21 ; N#Cc1ccc(-n2c3ccccc3c3c2ccc2c4ccccc4n(-c4ccccc4)c23)c(-c2ccccc2-n2c3ccccc3c3ccccc32)c1]

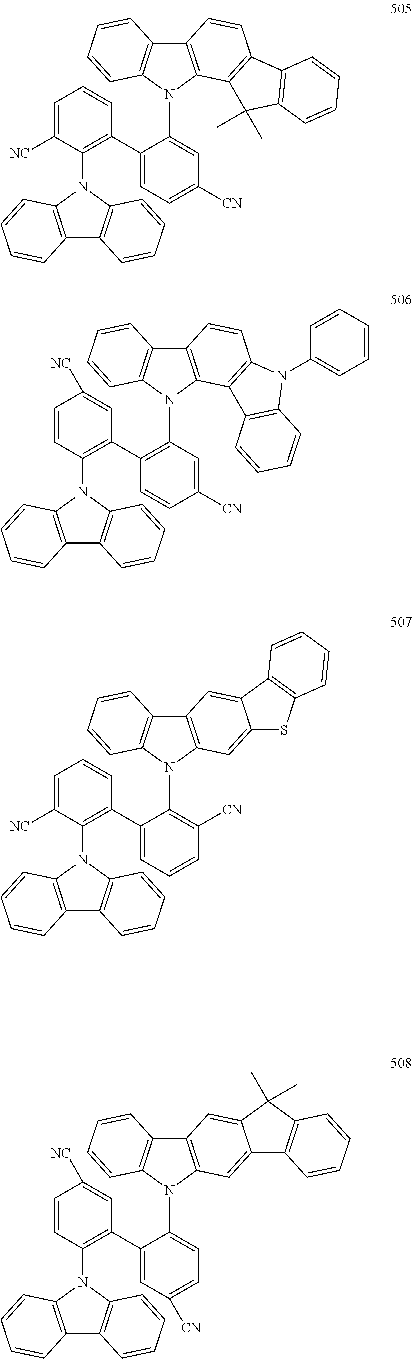 [SMILES: CC1(C)c2ccccc2-c2ccc3c4ccccc4n(-c4cc(C#N)ccc4-c4cccc(C#N)c4-n4c5ccccc5c5ccccc54)c3c21 ; N#Cc1ccc(-n2c3ccccc3c3ccccc32)c(-c2ccc(C#N)cc2-n2c3ccccc3c3ccc4c(c5ccccc5n4-c4ccccc4)c32)c1 ; N#Cc1cccc(-c2cccc(C#N)c2-n2c3ccccc3c3cc4c(cc32)sc2ccccc24)c1-n1c2ccccc2c2ccccc21 ; CC1(C)c2ccccc2-c2cc3c(cc21)c1ccccc1n3-c1ccc(C#N)cc1-c1cc(C#N)ccc1-n1c2ccccc2c2ccccc21]

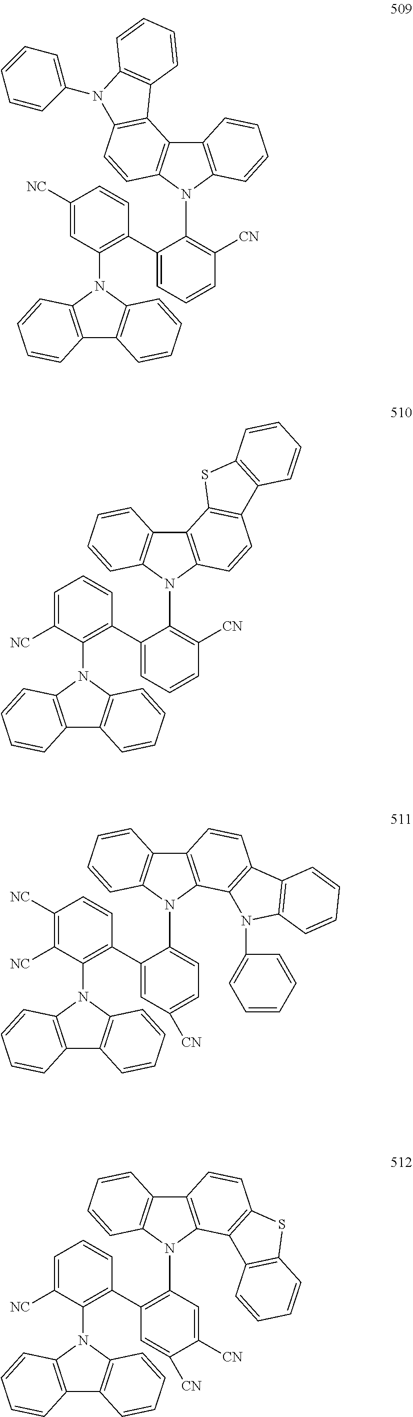 [SMILES: N#Cc1ccc(-c2cccc(C#N)c2-n2c3ccccc3c3c4c5ccccc5n(-c5ccccc5)c4ccc32)c(-n2c3ccccc3c3ccccc32)c1 ; N#Cc1cccc(-c2cccc(C#N)c2-n2c3ccccc3c3c4sc5ccccc5c4ccc32)c1-n1c2ccccc2c2ccccc21 ; N#Cc1ccc(-n2c3ccccc3c3ccc4c5ccccc5n(-c5ccccc5)c4c32)c(-c2ccc(C#N)c(C#N)c2-n2c3ccccc3c3ccccc32)c1 ; N#Cc1cc(-c2cccc(C#N)c2-n2c3ccccc3c3ccccc32)c(-n2c3ccccc3c3ccc4sc5ccccc5c4c32)cc1C#N]

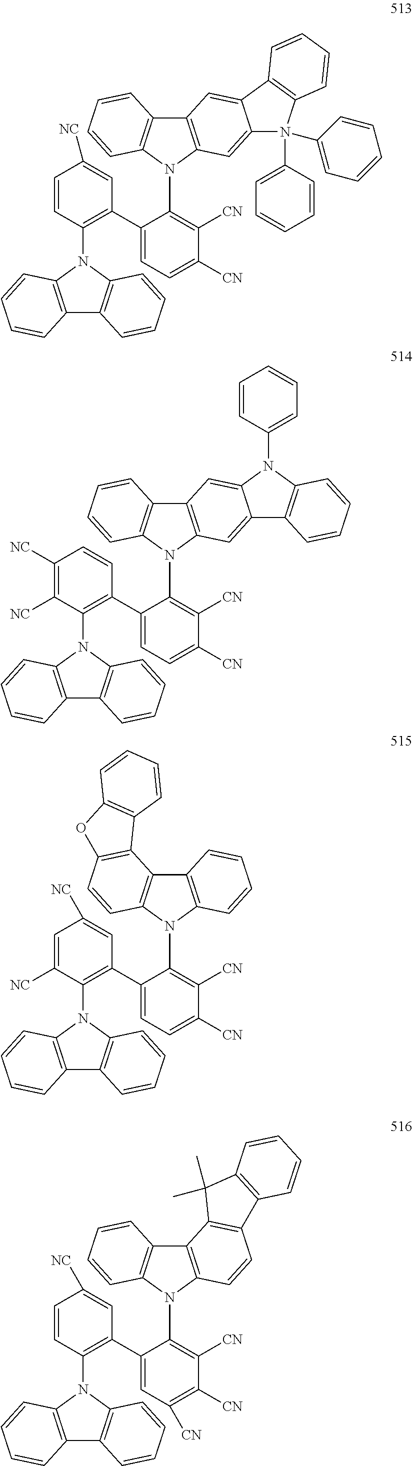

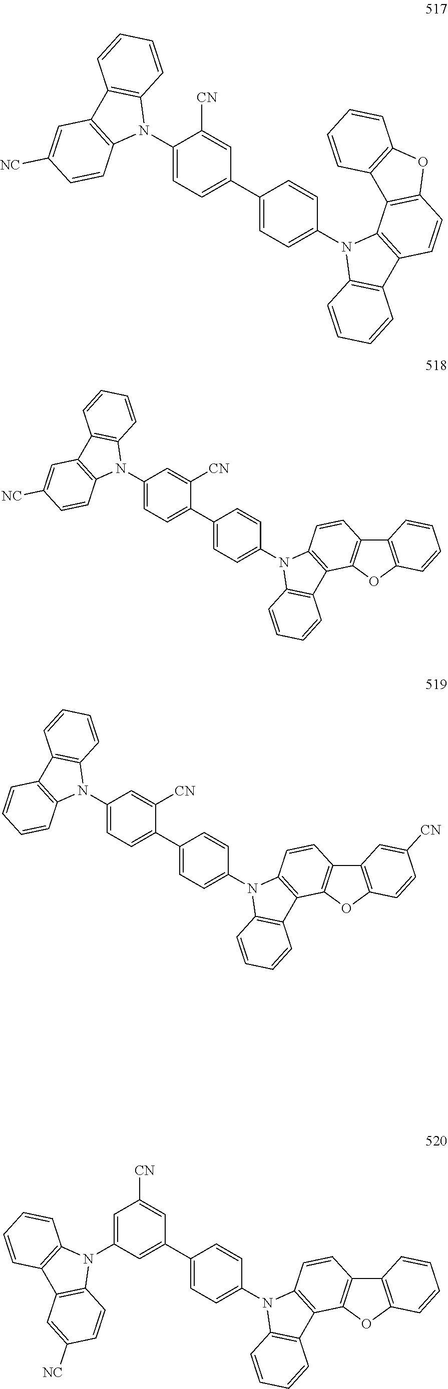 [SMILES: N#Cc1ccc2c(c1)c1ccccc1n2-c1ccc(-c2ccc(-n3c4ccccc4c4ccc5oc6ccccc6c5c43)cc2)cc1C#N ; N#Cc1ccc2c(c1)c1ccccc1n2-c1ccc(-c2ccc(-n3c4ccccc4c4c5oc6ccccc6c5ccc43)cc2)c(C#N)c1 ; N#Cc1ccc2oc3c(ccc4c3c3ccccc3n4-c3ccc(-c4ccc(-n5c6ccccc6c6ccccc65)cc4C#N)cc3)c2c1 ; N#Cc1cc(-c2ccc(-n3c4ccccc4c4c5oc6ccccc6c5ccc43)cc2)cc(-n2c3ccccc3c3cc(C#N)ccc32)c1]

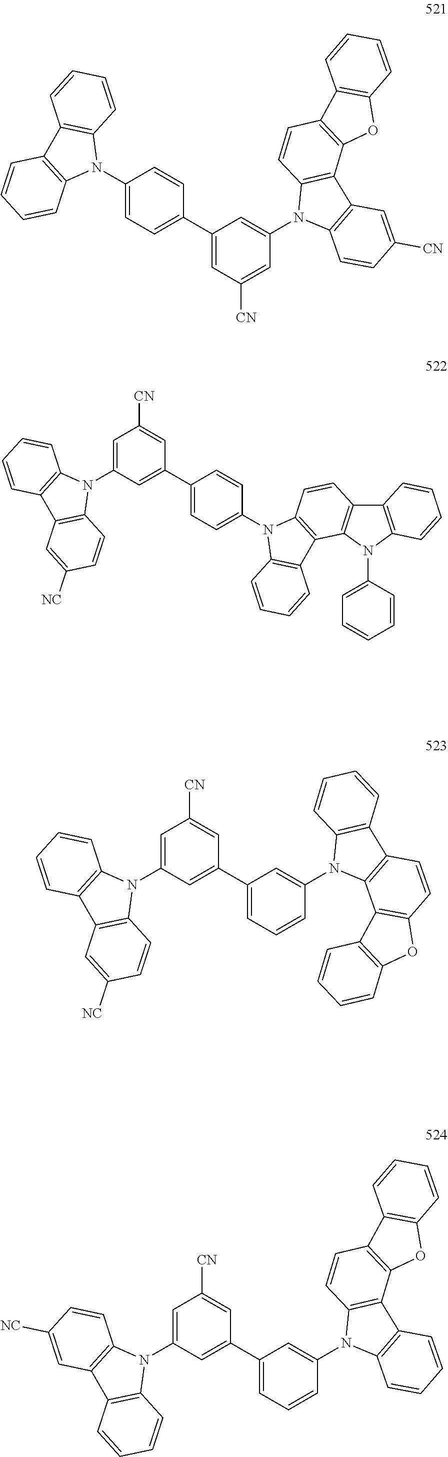 [SMILES: N#Cc1cc(-c2ccc(-n3c4ccccc4c4ccccc43)cc2)cc(-n2c3ccc(C#N)cc3c3c4oc5ccccc5c4ccc32)c1 ; N#Cc1cc(-c2ccc(-n3c4ccccc4c4c3ccc3c5ccccc5n(-c5ccccc5)c34)cc2)cc(-n2c3ccccc3c3cc(C#N)ccc32)c1 ; N#Cc1cc(-c2cccc(-n3c4ccccc4c4ccc5oc6ccccc6c5c43)c2)cc(-n2c3ccccc3c3cc(C#N)ccc32)c1 ; N#Cc1cc(-c2cccc(-n3c4ccccc4c4c5oc6ccccc6c5ccc43)c2)cc(-n2c3ccccc3c3cc(C#N)ccc32)c1]

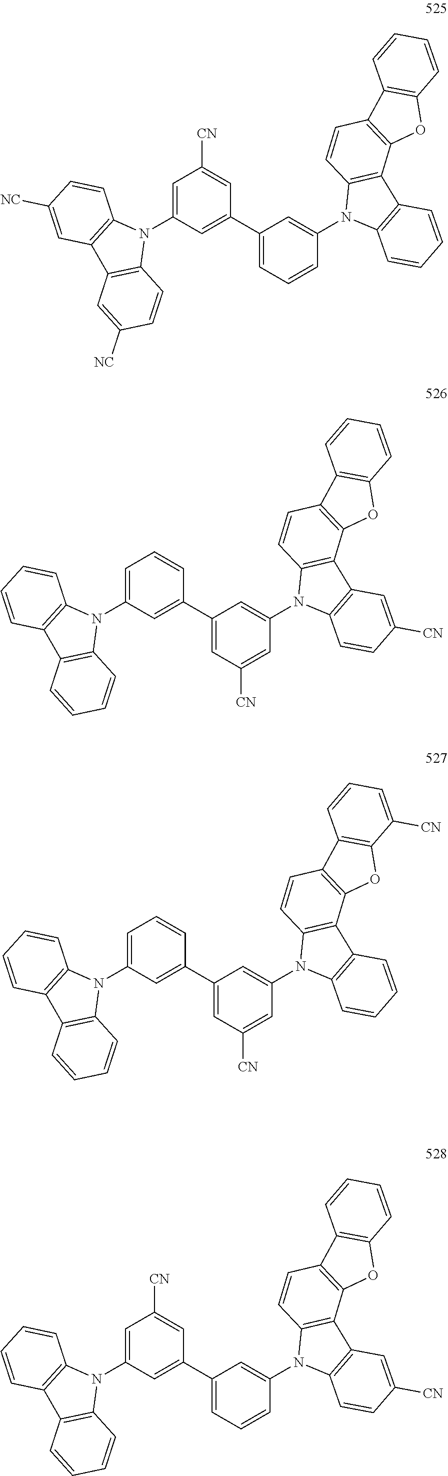 [SMILES: N#Cc1cc(-c2cccc(-n3c4ccccc4c4c5oc6ccccc6c5ccc43)c2)cc(-n2c3ccc(C#N)cc3c3cc(C#N)ccc32)c1 ; N#Cc1cc(-c2cccc(-n3c4ccccc4c4ccccc43)c2)cc(-n2c3ccc(C#N)cc3c3c4oc5ccccc5c4ccc32)c1 ; N#Cc1cc(-c2cccc(-n3c4ccccc4c4ccccc43)c2)cc(-n2c3ccccc3c3c4oc5c(C#N)cccc5c4ccc32)c1 ; N#Cc1cc(-c2cccc(-n3c4ccc(C#N)cc4c4c5oc6ccccc6c5ccc43)c2)cc(-n2c3ccccc3c3ccccc32)c1]

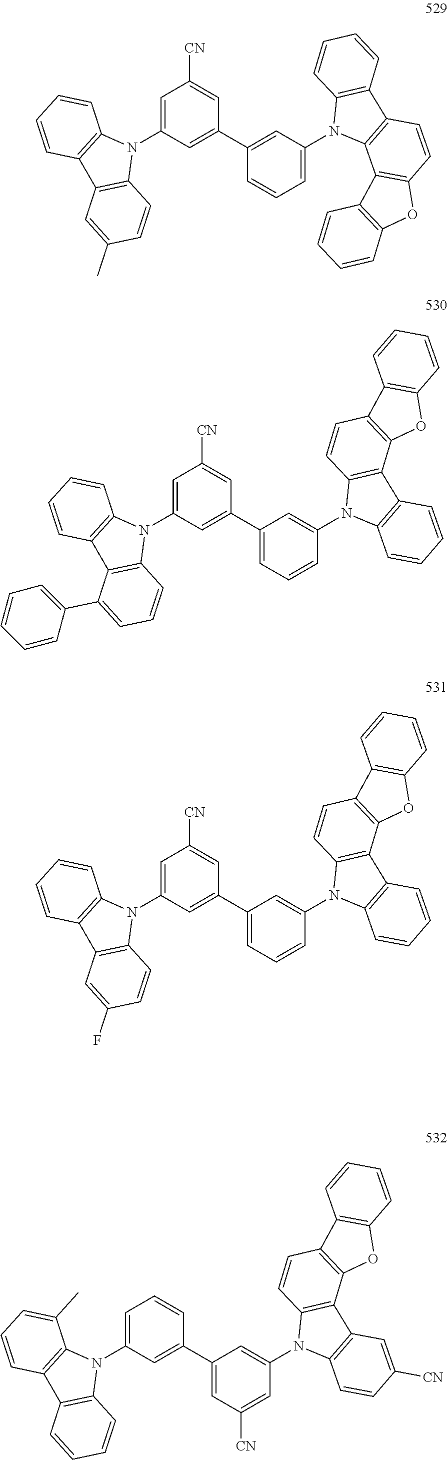 [SMILES: Cc1ccc2c(c1)c1ccccc1n2-c1cc(C#N)cc(-c2cccc(-n3c4ccccc4c4ccc5oc6ccccc6c5c43)c2)c1 ; N#Cc1cc(-c2cccc(-n3c4ccccc4c4c5oc6ccccc6c5ccc43)c2)cc(-n2c3ccccc3c3c(-c4ccccc4)cccc32)c1 ; N#Cc1cc(-c2cccc(-n3c4ccccc4c4c5oc6ccccc6c5ccc43)c2)cc(-n2c3ccccc3c3cc(F)ccc32)c1 ; Cc1cccc2c3ccccc3n(-c3cccc(-c4cc(C#N)cc(-n5c6ccc(C#N)cc6c6c7oc8ccccc8c7ccc65)c4)c3)c12]

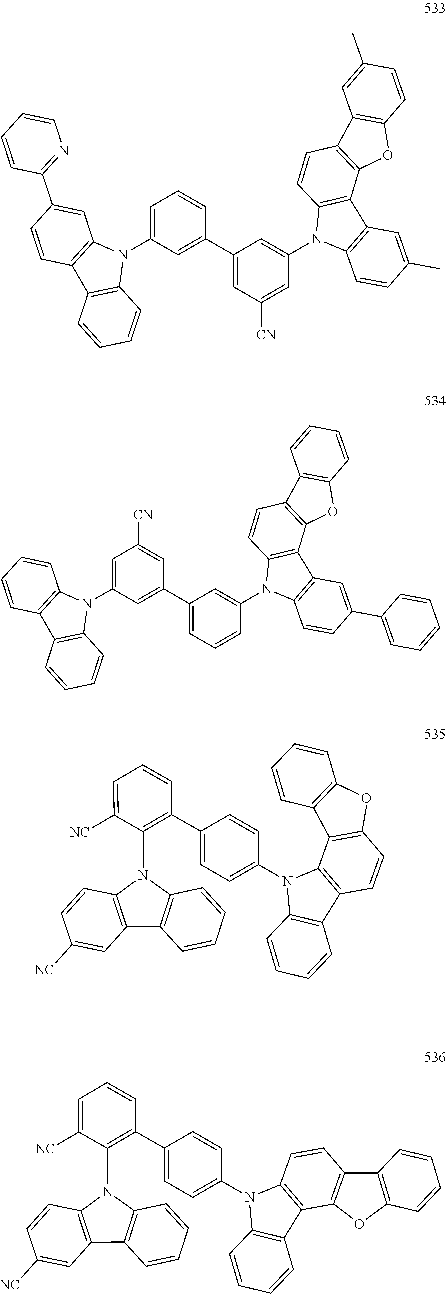 [SMILES: Cc1ccc2oc3c(ccc4c3c3cc(C)ccc3n4-c3cc(C#N)cc(-c4cccc(-n5c6ccccc6c6ccc(-c7ccccn7)cc65)c4)c3)c2c1 ; N#Cc1cc(-c2cccc(-n3c4ccc(-c5ccccc5)cc4c4c5oc6ccccc6c5ccc43)c2)cc(-n2c3ccccc3c3ccccc32)c1 ; N#Cc1ccc2c(c1)c1ccccc1n2-c1c(C#N)cccc1-c1ccc(-n2c3ccccc3c3ccc4oc5ccccc5c4c32)cc1 ; N#Cc1ccc2c(c1)c1ccccc1n2-c1c(C#N)cccc1-c1ccc(-n2c3ccccc3c3c4oc5ccccc5c4ccc32)cc1]

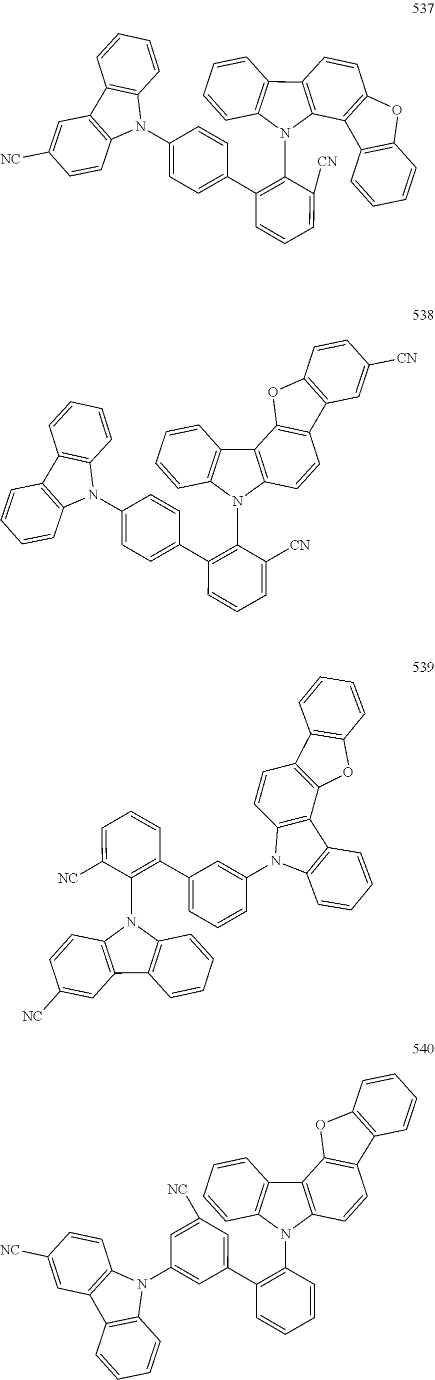 [SMILES: N#Cc1ccc2c(c1)c1ccccc1n2-c1ccc(-c2cccc(C#N)c2-n2c3ccccc3c3ccc4oc5ccccc5c4c32)cc1 ; N#Cc1ccc2oc3c(ccc4c3c3ccccc3n4-c3c(C#N)cccc3-c3ccc(-n4c5ccccc5c5ccccc54)cc3)c2c1 ; N#Cc1ccc2c(c1)c1ccccc1n2-c1c(C#N)cccc1-c1cccc(-n2c3ccccc3c3c4oc5ccccc5c4ccc32)c1 ; N#Cc1cc(-c2ccccc2-n2c3ccccc3c3c4oc5ccccc5c4ccc32)cc(-n2c3ccccc3c3cc(C#N)ccc32)c1]

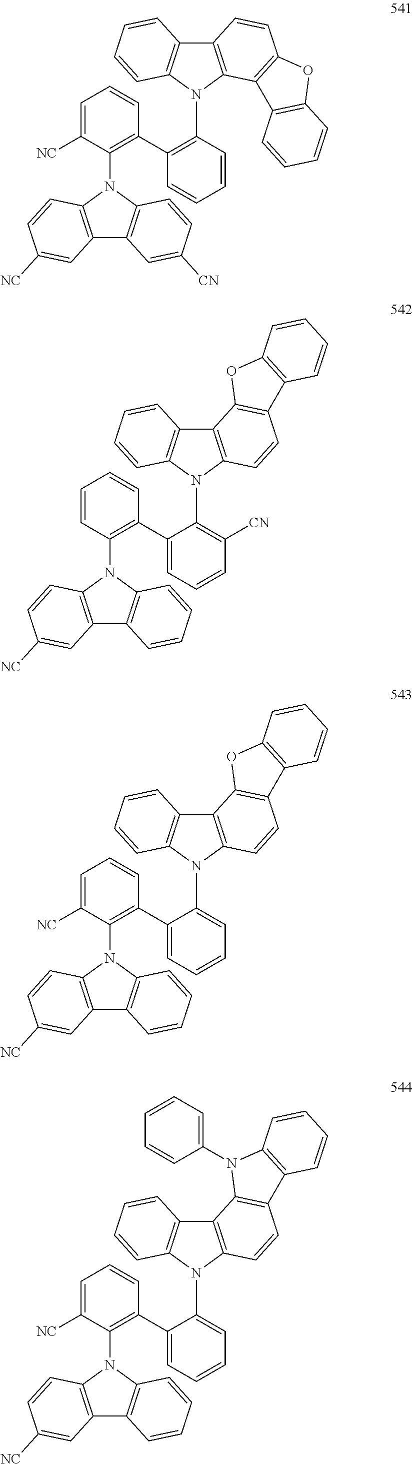 [SMILES: N#Cc1ccc2c(c1)c1cc(C#N)ccc1n2-c1c(C#N)cccc1-c1ccccc1-n1c2ccccc2c2ccc3oc4ccccc4c3c21 ; N#Cc1ccc2c(c1)c1ccccc1n2-c1ccccc1-c1cccc(C#N)c1-n1c2ccccc2c2c3oc4ccccc4c3ccc21 ; N#Cc1ccc2c(c1)c1ccccc1n2-c1c(C#N)cccc1-c1ccccc1-n1c2ccccc2c2c3oc4ccccc4c3ccc21 ; N#Cc1ccc2c(c1)c1ccccc1n2-c1c(C#N)cccc1-c1ccccc1-n1c2ccccc2c2c1ccc1c3ccccc3n(-c3ccccc3)c12]

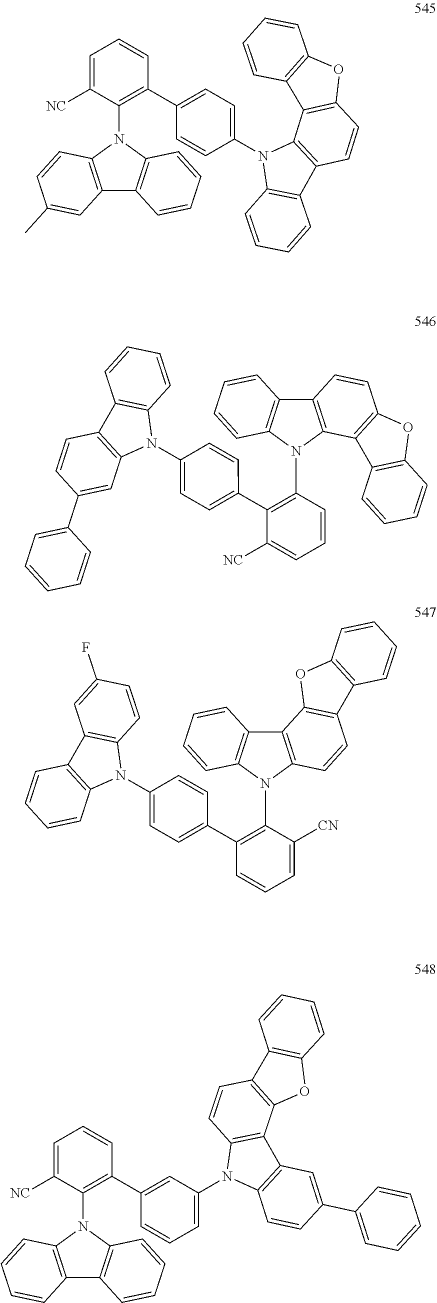 [SMILES: Cc1ccc2c(c1)c1ccccc1n2-c1c(C#N)cccc1-c1ccc(-n2c3ccccc3c3ccc4oc5ccccc5c4c32)cc1 ; N#Cc1cccc(-n2c3ccccc3c3ccc4oc5ccccc5c4c32)c1-c1ccc(-n2c3ccccc3c3ccc(-c4ccccc4)cc32)cc1 ; N#Cc1cccc(-c2ccc(-n3c4ccccc4c4cc(F)ccc43)cc2)c1-n1c2ccccc2c2c3oc4ccccc4c3ccc21 ; N#Cc1cccc(-c2cccc(-n3c4ccc(-c5ccccc5)cc4c4c5oc6ccccc6c5ccc43)c2)c1-n1c2ccccc2c2ccccc21]

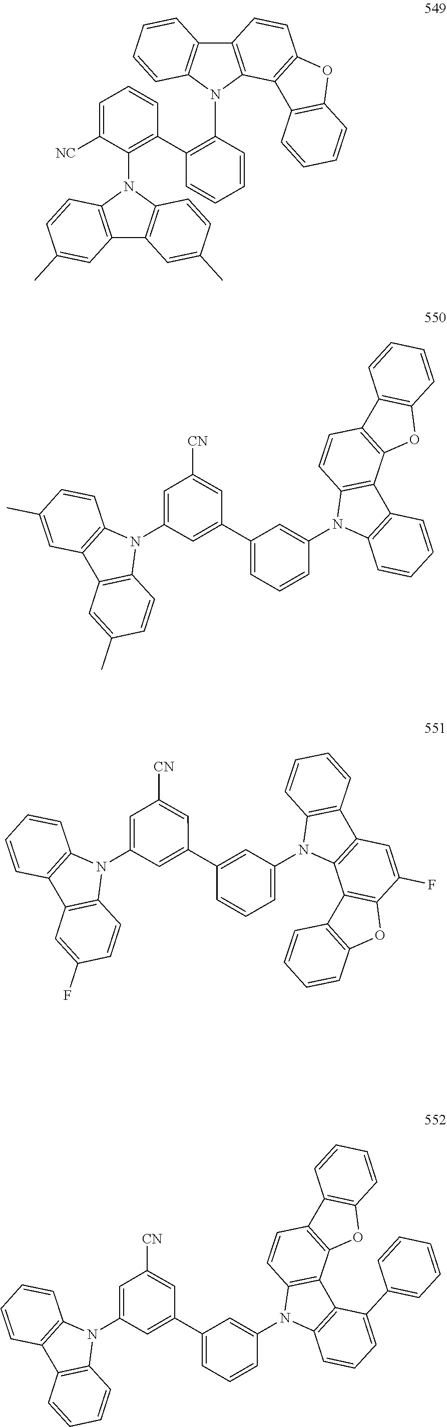 [SMILES: Cc1ccc2c(c1)c1cc(C)ccc1n2-c1c(C#N)cccc1-c1ccccc1-n1c2ccccc2c2ccc3oc4ccccc4c3c21 ; Cc1ccc2c(c1)c1cc(C)ccc1n2-c1cc(C#N)cc(-c2cccc(-n3c4ccccc4c4c5oc6ccccc6c5ccc43)c2)c1 ; N#Cc1cc(-c2cccc(-n3c4ccccc4c4cc(F)c5oc6ccccc6c5c43)c2)cc(-n2c3ccccc3c3cc(F)ccc32)c1 ; N#Cc1cc(-c2cccc(-n3c4cccc(-c5ccccc5)c4c4c5oc6ccccc6c5ccc43)c2)cc(-n2c3ccccc3c3ccccc32)c1]

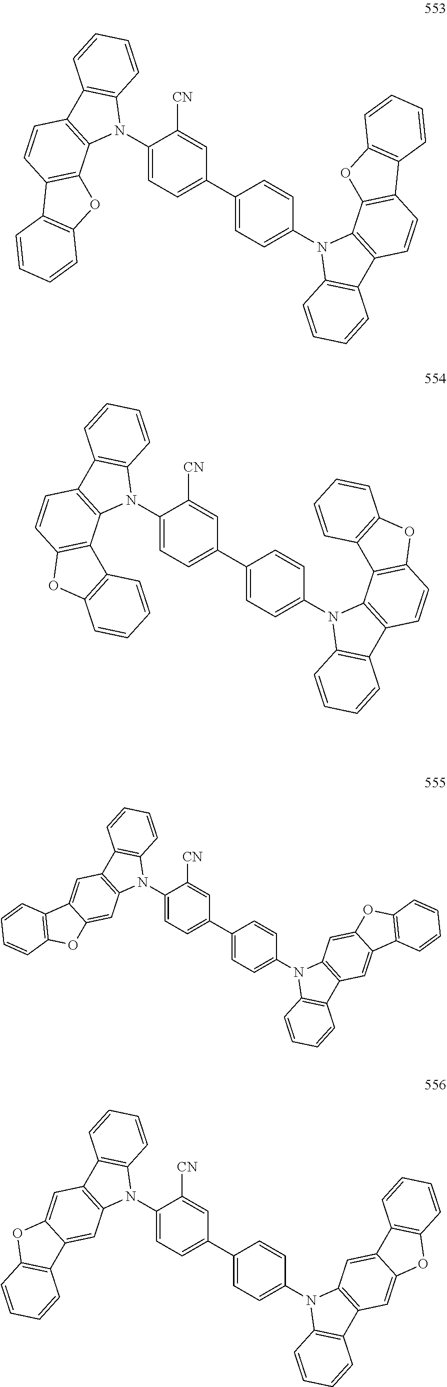 [SMILES: N#Cc1cc(-c2ccc(-n3c4ccccc4c4ccc5c6ccccc6oc5c43)cc2)ccc1-n1c2ccccc2c2ccc3c4ccccc4oc3c21 ; N#Cc1cc(-c2ccc(-n3c4ccccc4c4ccc5oc6ccccc6c5c43)cc2)ccc1-n1c2ccccc2c2ccc3oc4ccccc4c3c21 ; N#Cc1cc(-c2ccc(-n3c4ccccc4c4cc5c(cc43)oc3ccccc35)cc2)ccc1-n1c2ccccc2c2cc3c(cc21)oc1ccccc13 ; N#Cc1cc(-c2ccc(-n3c4ccccc4c4cc5oc6ccccc6c5cc43)cc2)ccc1-n1c2ccccc2c2cc3oc4ccccc4c3cc21]

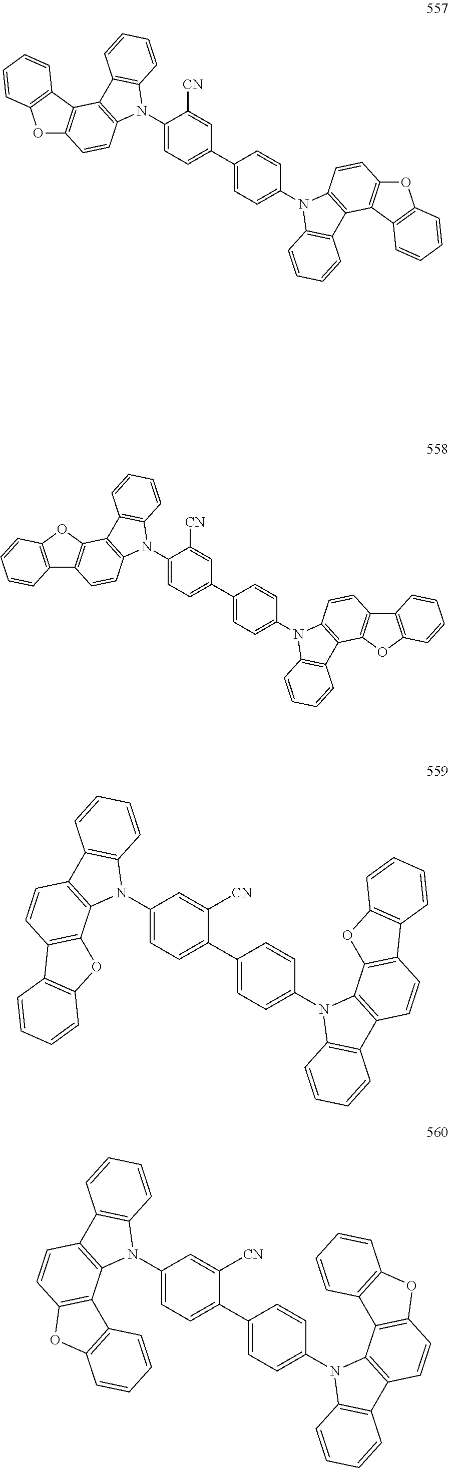 [SMILES: N#Cc1cc(-c2ccc(-n3c4ccccc4c4c5c(ccc43)oc3ccccc35)cc2)ccc1-n1c2ccccc2c2c3c(ccc21)oc1ccccc13 ; N#Cc1cc(-c2ccc(-n3c4ccccc4c4c5oc6ccccc6c5ccc43)cc2)ccc1-n1c2ccccc2c2c3oc4ccccc4c3ccc21 ; N#Cc1cc(-n2c3ccccc3c3ccc4c5ccccc5oc4c32)ccc1-c1ccc(-n2c3ccccc3c3ccc4c5ccccc5oc4c32)cc1 ; N#Cc1cc(-n2c3ccccc3c3ccc4oc5ccccc5c4c32)ccc1-c1ccc(-n2c3ccccc3c3ccc4oc5ccccc5c4c32)cc1]

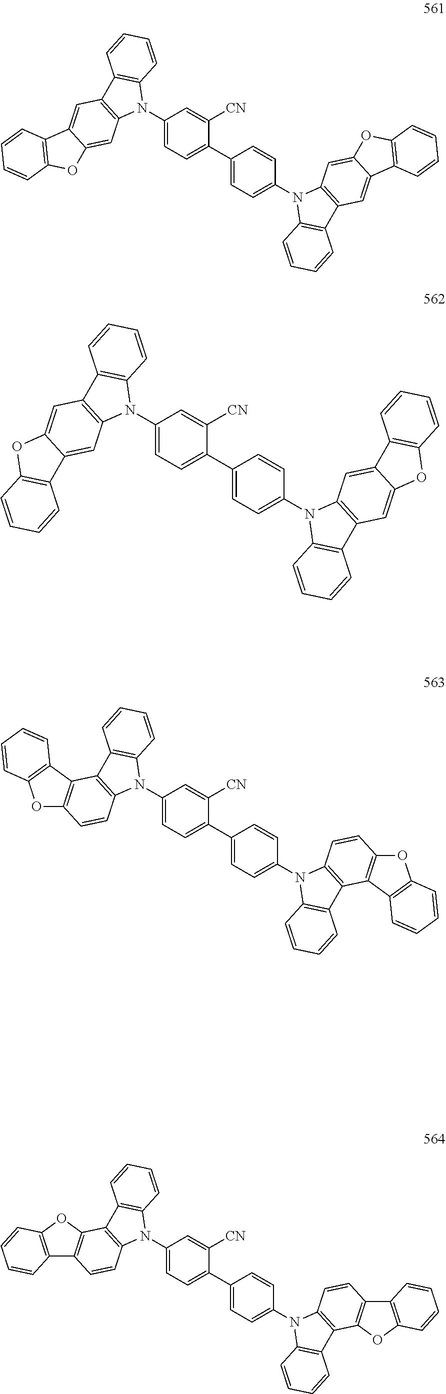 [SMILES: N#Cc1cc(-n2c3ccccc3c3cc4c(cc32)oc2ccccc24)ccc1-c1ccc(-n2c3ccccc3c3cc4c(cc32)oc2ccccc24)cc1 ; N#Cc1cc(-n2c3ccccc3c3cc4oc5ccccc5c4cc32)ccc1-c1ccc(-n2c3ccccc3c3cc4oc5ccccc5c4cc32)cc1 ; N#Cc1cc(-n2c3ccccc3c3c4c(ccc32)oc2ccccc24)ccc1-c1ccc(-n2c3ccccc3c3c4c(ccc32)oc2ccccc24)cc1 ; N#Cc1cc(-n2c3ccccc3c3c4oc5ccccc5c4ccc32)ccc1-c1ccc(-n2c3ccccc3c3c4oc5ccccc5c4ccc32)cc1]

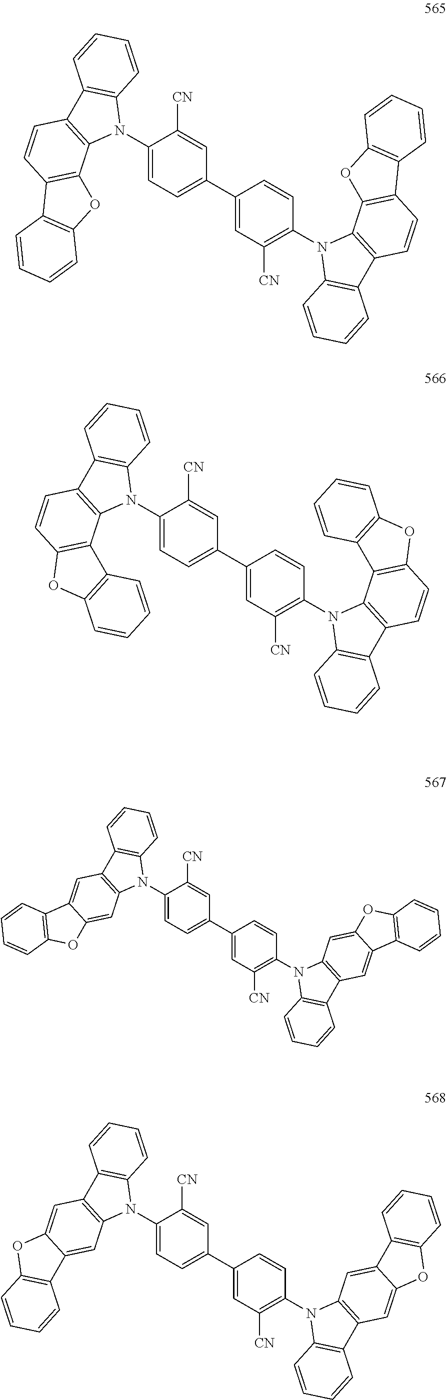 [SMILES: N#Cc1cc(-c2ccc(-n3c4ccccc4c4ccc5c6ccccc6oc5c43)c(C#N)c2)ccc1-n1c2ccccc2c2ccc3c4ccccc4oc3c21 ; N#Cc1cc(-c2ccc(-n3c4ccccc4c4ccc5oc6ccccc6c5c43)c(C#N)c2)ccc1-n1c2ccccc2c2ccc3oc4ccccc4c3c21 ; N#Cc1cc(-c2ccc(-n3c4ccccc4c4cc5c(cc43)oc3ccccc35)c(C#N)c2)ccc1-n1c2ccccc2c2cc3c(cc21)oc1ccccc13 ; N#Cc1cc(-c2ccc(-n3c4ccccc4c4cc5oc6ccccc6c5cc43)c(C#N)c2)ccc1-n1c2ccccc2c2cc3oc4ccccc4c3cc21]

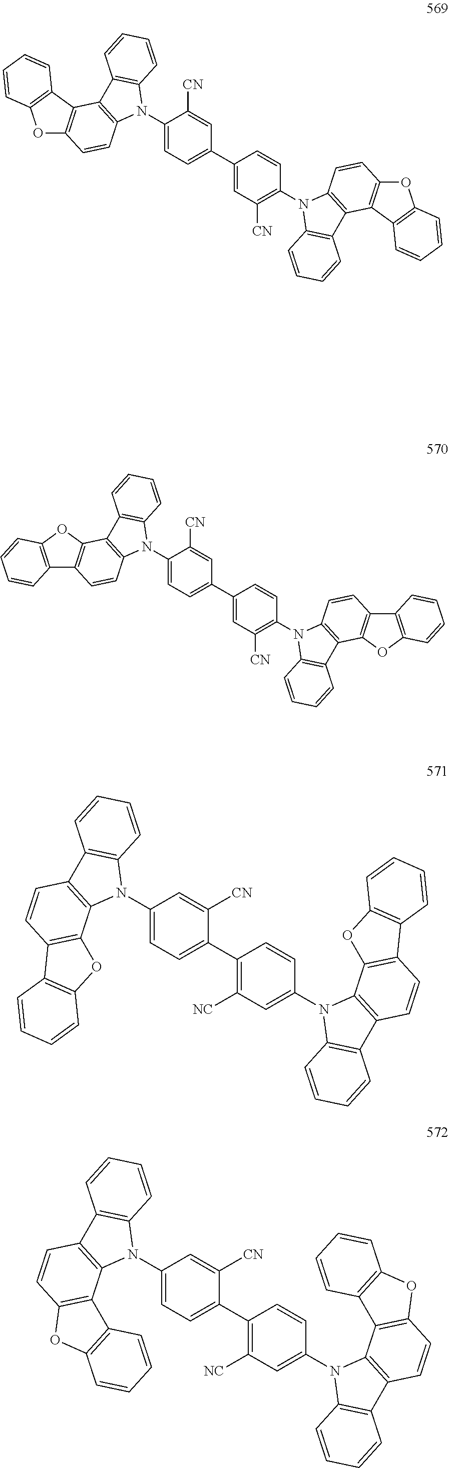 [SMILES: N#Cc1cc(-c2ccc(-n3c4ccccc4c4c5c(ccc43)oc3ccccc35)c(C#N)c2)ccc1-n1c2ccccc2c2c3c(ccc21)oc1ccccc13 ; N#Cc1cc(-c2ccc(-n3c4ccccc4c4c5oc6ccccc6c5ccc43)c(C#N)c2)ccc1-n1c2ccccc2c2c3oc4ccccc4c3ccc21 ; N#Cc1cc(-n2c3ccccc3c3ccc4c5ccccc5oc4c32)ccc1-c1ccc(-n2c3ccccc3c3ccc4c5ccccc5oc4c32)cc1C#N ; N#Cc1cc(-n2c3ccccc3c3ccc4oc5ccccc5c4c32)ccc1-c1ccc(-n2c3ccccc3c3ccc4oc5ccccc5c4c32)cc1C#N]

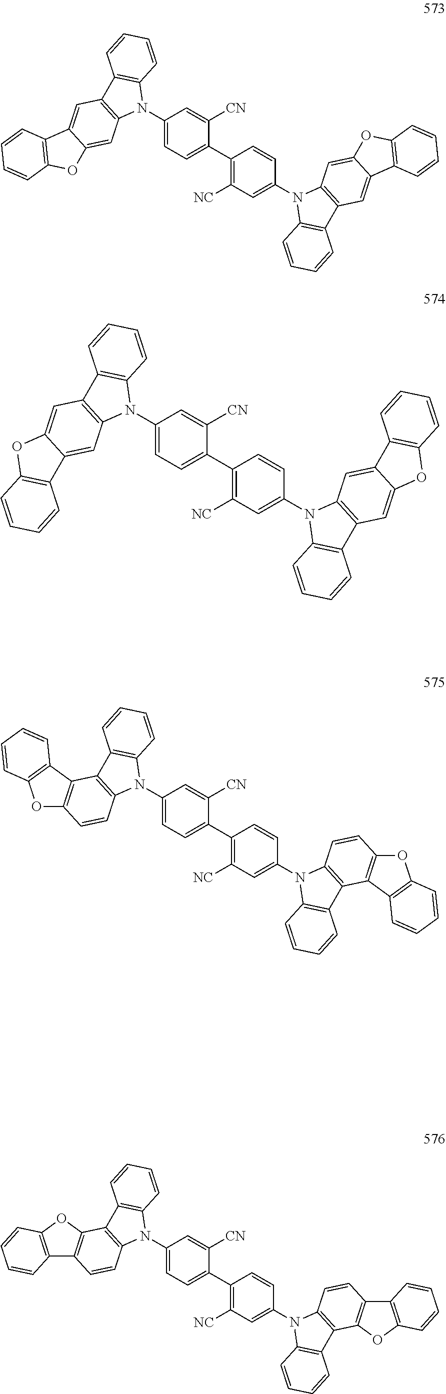 [SMILES: N#Cc1cc(-n2c3ccccc3c3cc4c(cc32)oc2ccccc24)ccc1-c1ccc(-n2c3ccccc3c3cc4c(cc32)oc2ccccc24)cc1C#N ; N#Cc1cc(-n2c3ccccc3c3cc4oc5ccccc5c4cc32)ccc1-c1ccc(-n2c3ccccc3c3cc4oc5ccccc5c4cc32)cc1C#N ; N#Cc1cc(-n2c3ccccc3c3c4c(ccc32)oc2ccccc24)ccc1-c1ccc(-n2c3ccccc3c3c4c(ccc32)oc2ccccc24)cc1C#N ; N#Cc1cc(-n2c3ccccc3c3c4oc5ccccc5c4ccc32)ccc1-c1ccc(-n2c3ccccc3c3c4oc5ccccc5c4ccc32)cc1C#N]

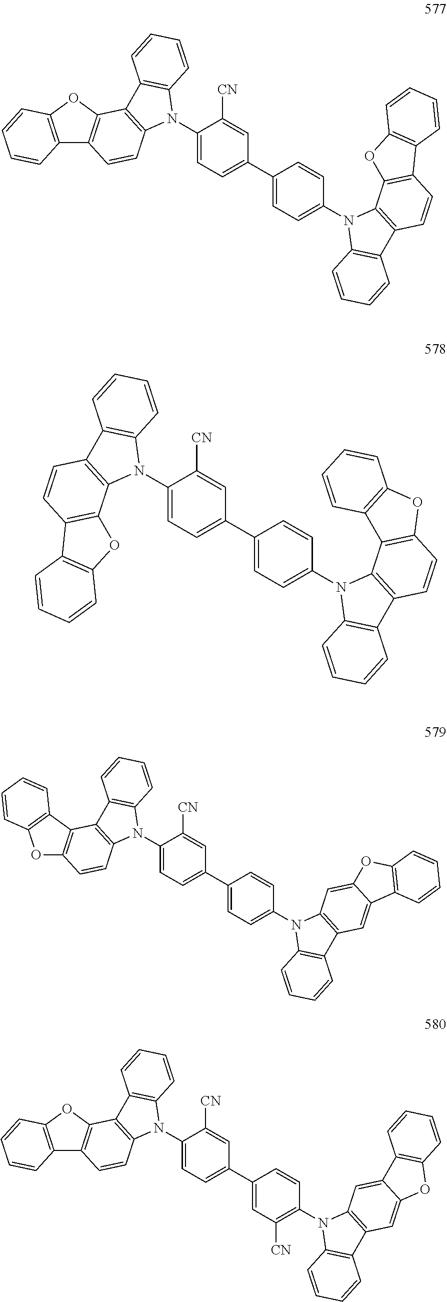 [SMILES: N#Cc1cc(-c2ccc(-n3c4ccccc4c4ccc5c6ccccc6oc5c43)cc2)ccc1-n1c2ccccc2c2c3oc4ccccc4c3ccc21 ; N#Cc1cc(-c2ccc(-n3c4ccccc4c4ccc5oc6ccccc6c5c43)cc2)ccc1-n1c2ccccc2c2ccc3c4ccccc4oc3c21 ; N#Cc1cc(-c2ccc(-n3c4ccccc4c4cc5c(cc43)oc3ccccc35)cc2)ccc1-n1c2ccccc2c2c3c(ccc21)oc1ccccc13 ; N#Cc1cc(-c2ccc(-n3c4ccccc4c4c5oc6ccccc6c5ccc43)c(C#N)c2)ccc1-n1c2ccccc2c2cc3oc4ccccc4c3cc21]

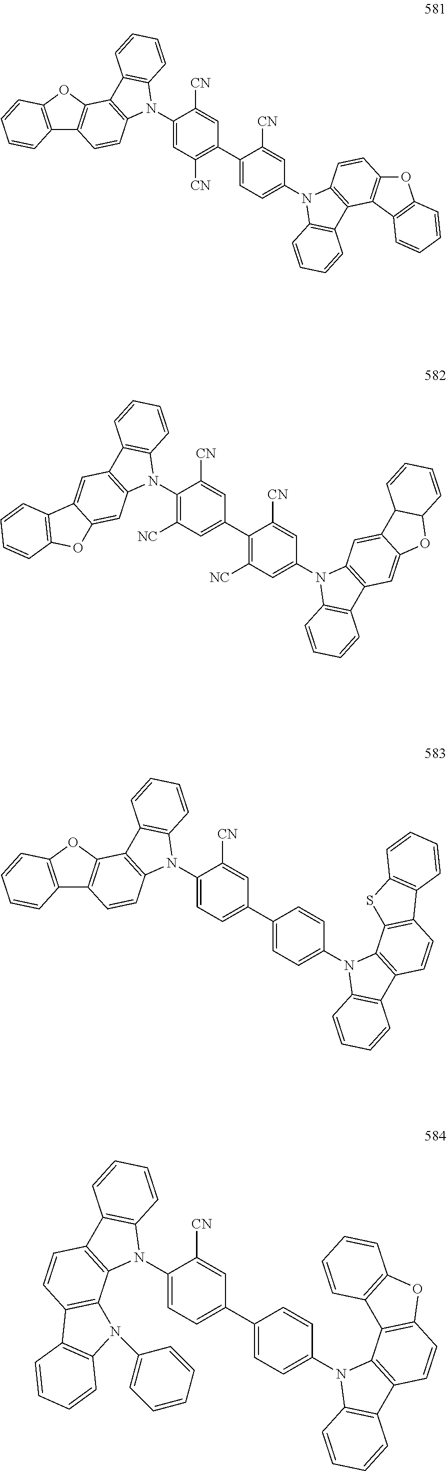 [SMILES: N#Cc1cc(-n2c3ccccc3c3c4c(ccc32)oc2ccccc24)ccc1-c1cc(C#N)c(-n2c3ccccc3c3c4oc5ccccc5c4ccc32)cc1C#N ; N#Cc1cc(-n2c3ccccc3c3cc4c(cc32)C2C=CC=CC2O4)cc(C#N)c1-c1cc(C#N)c(-n2c3ccccc3c3cc4c(cc32)oc2ccccc24)c(C#N)c1 ; N#Cc1cc(-c2ccc(-n3c4ccccc4c4ccc5c6ccccc6sc5c43)cc2)ccc1-n1c2ccccc2c2c3oc4ccccc4c3ccc21 ; N#Cc1cc(-c2ccc(-n3c4ccccc4c4ccc5oc6ccccc6c5c43)cc2)ccc1-n1c2ccccc2c2ccc3c4ccccc4n(-c4ccccc4)c3c21]

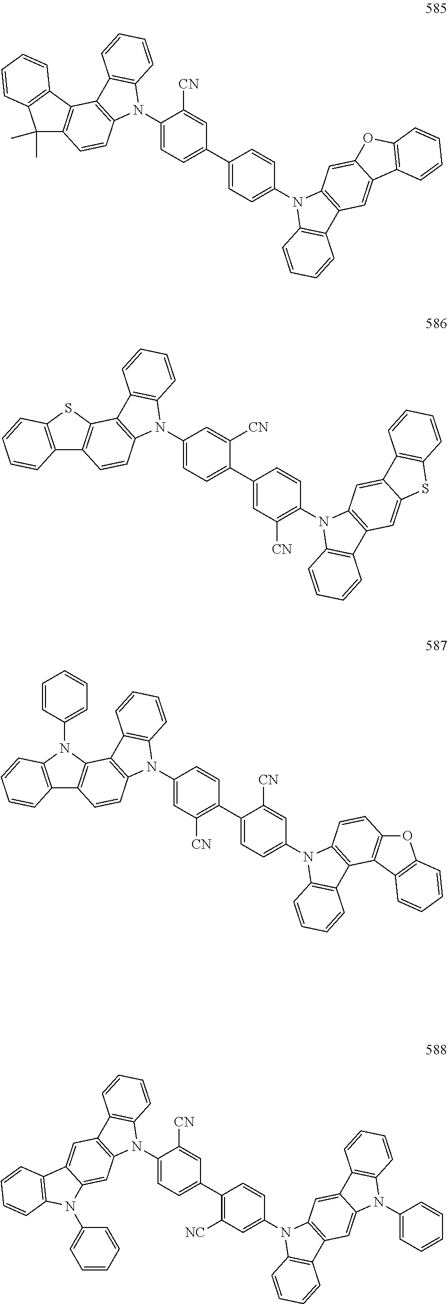 [SMILES: CC1(C)c2ccccc2-c2c1ccc1c2c2ccccc2n1-c1ccc(-c2ccc(-n3c4ccccc4c4cc5c(cc43)oc3ccccc35)cc2)cc1C#N ; N#Cc1cc(-n2c3ccccc3c3c4sc5ccccc5c4ccc32)ccc1-c1ccc(-n2c3ccccc3c3cc4sc5ccccc5c4cc32)c(C#N)c1 ; N#Cc1cc(-n2c3ccccc3c3c4c(ccc32)oc2ccccc24)ccc1-c1ccc(-n2c3ccccc3c3c2ccc2c4ccccc4n(-c4ccccc4)c23)cc1C#N ; N#Cc1cc(-n2c3ccccc3c3cc4c(cc32)c2ccccc2n4-c2ccccc2)ccc1-c1ccc(-n2c3ccccc3c3cc4c5ccccc5n(-c5ccccc5)c4cc32)c(C#N)c1]

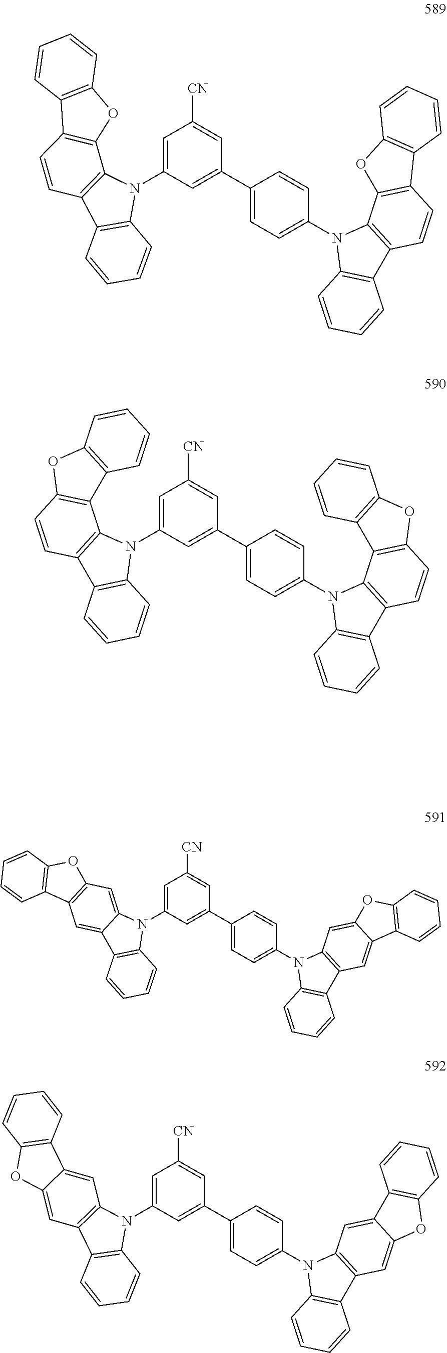 [SMILES: N#Cc1cc(-c2ccc(-n3c4ccccc4c4ccc5c6ccccc6oc5c43)cc2)cc(-n2c3ccccc3c3ccc4c5ccccc5oc4c32)c1 ; N#Cc1cc(-c2ccc(-n3c4ccccc4c4ccc5oc6ccccc6c5c43)cc2)cc(-n2c3ccccc3c3ccc4oc5ccccc5c4c32)c1 ; N#Cc1cc(-c2ccc(-n3c4ccccc4c4cc5c(cc43)oc3ccccc35)cc2)cc(-n2c3ccccc3c3cc4c(cc32)oc2ccccc24)c1 ; N#Cc1cc(-c2ccc(-n3c4ccccc4c4cc5oc6ccccc6c5cc43)cc2)cc(-n2c3ccccc3c3cc4oc5ccccc5c4cc32)c1]

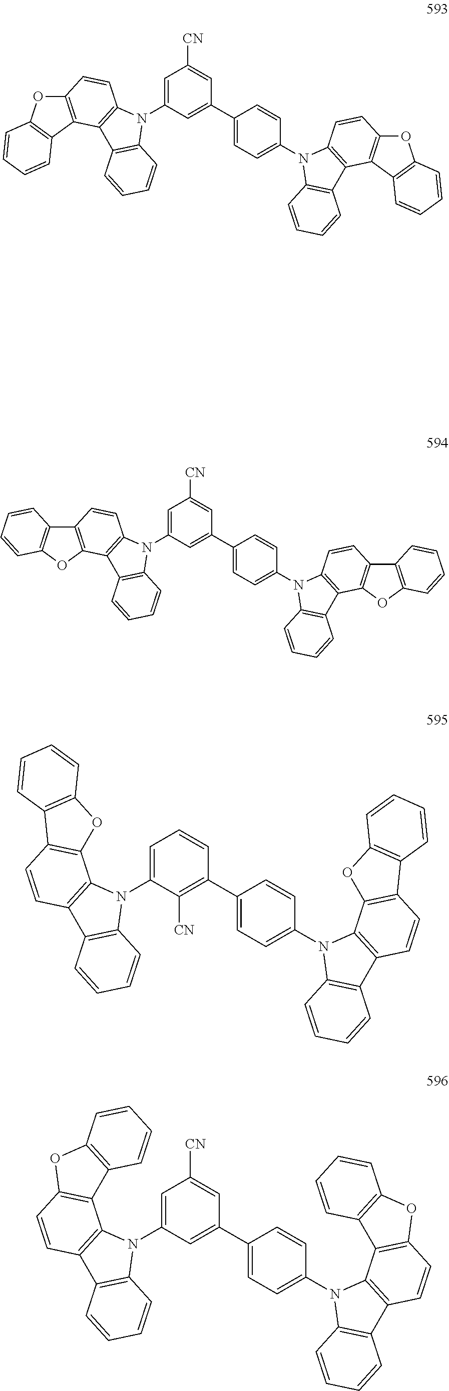 [SMILES: N#Cc1cc(-c2ccc(-n3c4ccccc4c4c5c(ccc43)oc3ccccc35)cc2)cc(-n2c3ccccc3c3c4c(ccc32)oc2ccccc24)c1 ; N#Cc1cc(-c2ccc(-n3c4ccccc4c4c5oc6ccccc6c5ccc43)cc2)cc(-n2c3ccccc3c3c4oc5ccccc5c4ccc32)c1 ; N#Cc1c(-c2ccc(-n3c4ccccc4c4ccc5c6ccccc6oc5c43)cc2)cccc1-n1c2ccccc2c2ccc3c4ccccc4oc3c21 ; N#Cc1cc(-c2ccc(-n3c4ccccc4c4ccc5oc6ccccc6c5c43)cc2)cc(-n2c3ccccc3c3ccc4oc5ccccc5c4c32)c1]

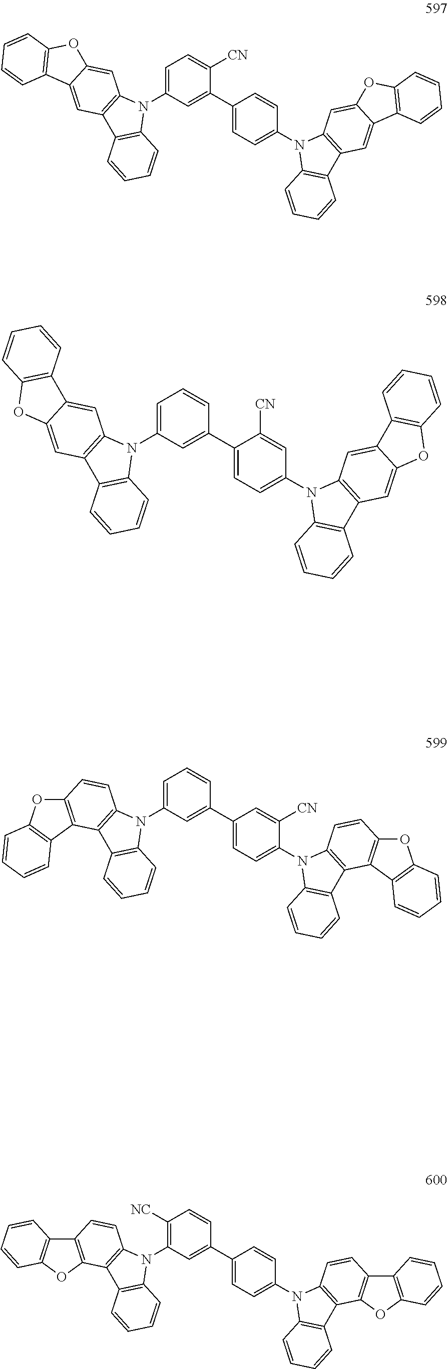 [SMILES: N#Cc1ccc(-n2c3ccccc3c3cc4c(cc32)oc2ccccc24)cc1-c1ccc(-n2c3ccccc3c3cc4c(cc32)oc2ccccc24)cc1 ; N#Cc1cc(-n2c3ccccc3c3cc4oc5ccccc5c4cc32)ccc1-c1cccc(-n2c3ccccc3c3cc4oc5ccccc5c4cc32)c1 ; N#Cc1cc(-c2cccc(-n3c4ccccc4c4c5c(ccc43)oc3ccccc35)c2)ccc1-n1c2ccccc2c2c3c(ccc21)oc1ccccc13 ; N#Cc1ccc(-c2ccc(-n3c4ccccc4c4c5oc6ccccc6c5ccc43)cc2)cc1-n1c2ccccc2c2c3oc4ccccc4c3ccc21]

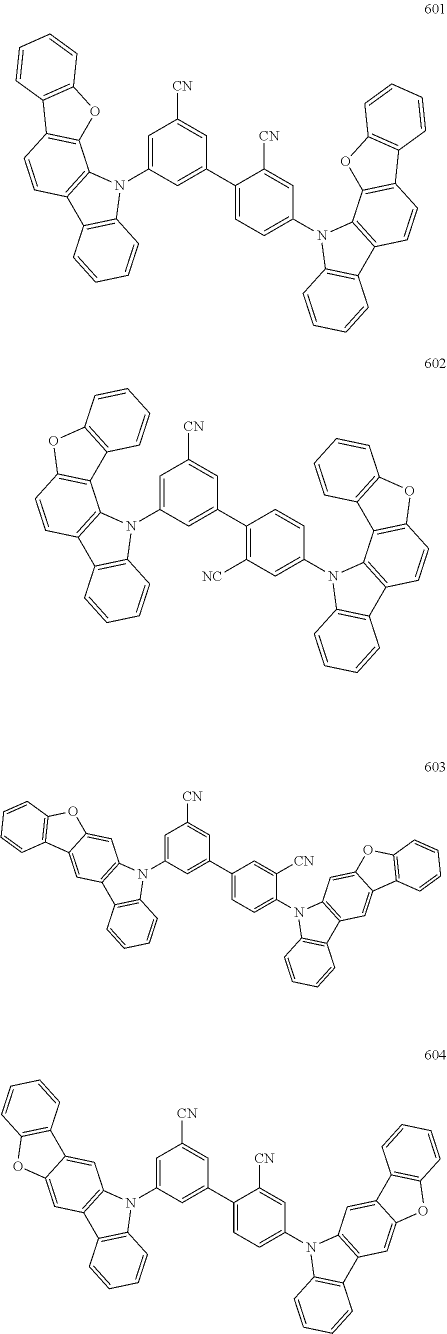 [SMILES: N#Cc1cc(-c2ccc(-n3c4ccccc4c4ccc5c6ccccc6oc5c43)cc2C#N)cc(-n2c3ccccc3c3ccc4c5ccccc5oc4c32)c1 ; N#Cc1cc(-c2ccc(-n3c4ccccc4c4ccc5oc6ccccc6c5c43)cc2C#N)cc(-n2c3ccccc3c3ccc4oc5ccccc5c4c32)c1 ; N#Cc1cc(-c2ccc(-n3c4ccccc4c4cc5c(cc43)oc3ccccc35)c(C#N)c2)cc(-n2c3ccccc3c3cc4c(cc32)oc2ccccc24)c1 ; N#Cc1cc(-c2ccc(-n3c4ccccc4c4cc5oc6ccccc6c5cc43)cc2C#N)cc(-n2c3ccccc3c3cc4oc5ccccc5c4cc32)c1]

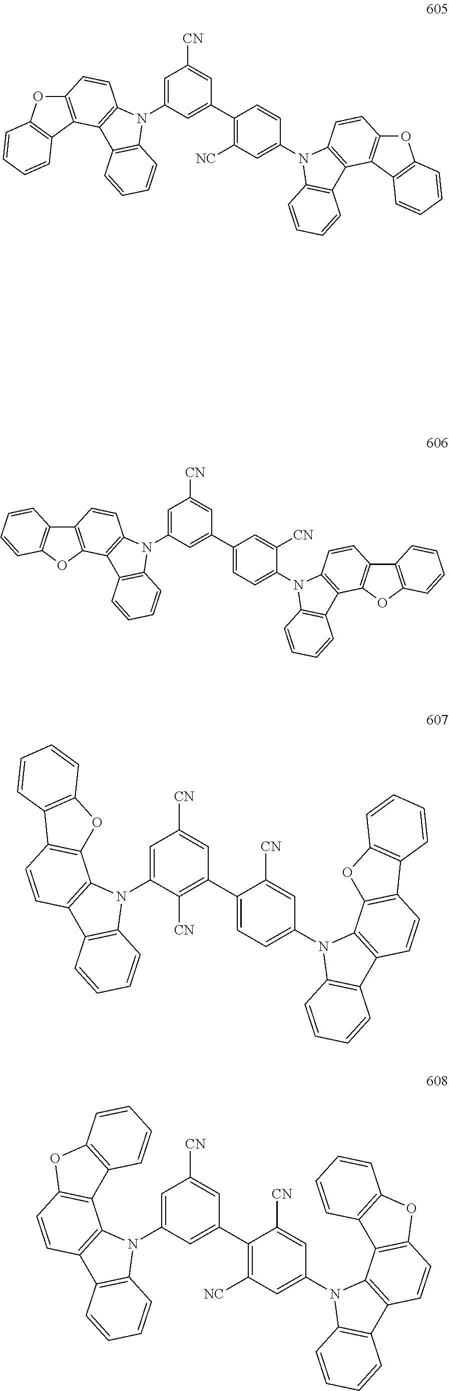 [SMILES: N#Cc1cc(-c2ccc(-n3c4ccccc4c4c5c(ccc43)oc3ccccc35)cc2C#N)cc(-n2c3ccccc3c3c4c(ccc32)oc2ccccc24)c1 ; N#Cc1cc(-c2ccc(-n3c4ccccc4c4c5oc6ccccc6c5ccc43)c(C#N)c2)cc(-n2c3ccccc3c3c4oc5ccccc5c4ccc32)c1 ; N#Cc1cc(-c2ccc(-n3c4ccccc4c4ccc5c6ccccc6oc5c43)cc2C#N)c(C#N)c(-n2c3ccccc3c3ccc4c5ccccc5oc4c32)c1 ; N#Cc1cc(-c2c(C#N)cc(-n3c4ccccc4c4ccc5oc6ccccc6c5c43)cc2C#N)cc(-n2c3ccccc3c3ccc4oc5ccccc5c4c32)c1]

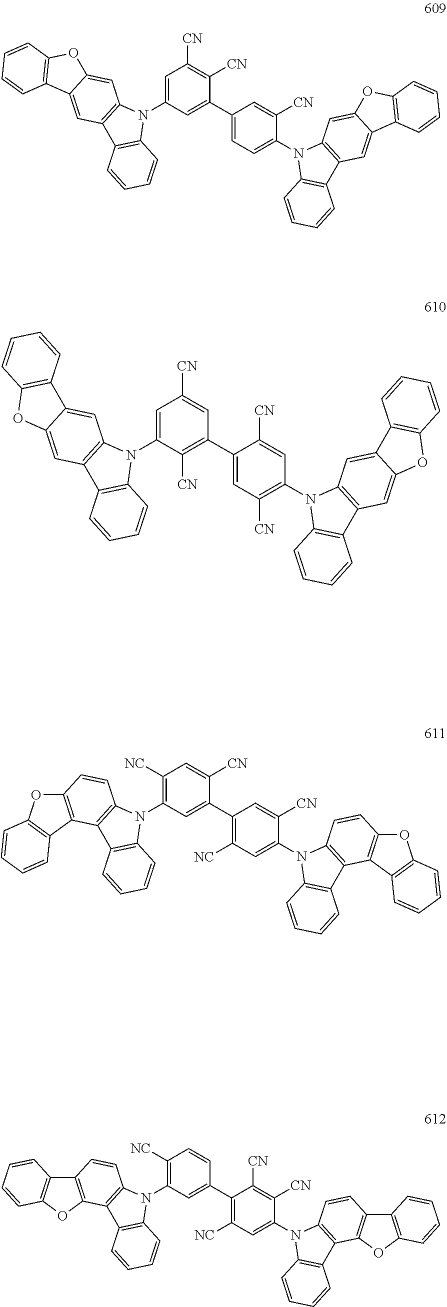 [SMILES: N#Cc1cc(-c2cc(-n3c4ccccc4c4cc5c(cc43)oc3ccccc35)cc(C#N)c2C#N)ccc1-n1c2ccccc2c2cc3c(cc21)oc1ccccc13 ; N#Cc1cc(-c2cc(C#N)c(-n3c4ccccc4c4cc5oc6ccccc6c5cc43)cc2C#N)c(C#N)c(-n2c3ccccc3c3cc4oc5ccccc5c4cc32)c1 ; N#Cc1cc(-n2c3ccccc3c3c4c(ccc32)oc2ccccc24)c(C#N)cc1-c1cc(-n2c3ccccc3c3c4c(ccc32)oc2ccccc24)c(C#N)cc1C#N ; N#Cc1ccc(-c2c(C#N)cc(-n3c4ccccc4c4c5oc6ccccc6c5ccc43)c(C#N)c2C#N)cc1-n1c2ccccc2c2c3oc4ccccc4c3ccc21]

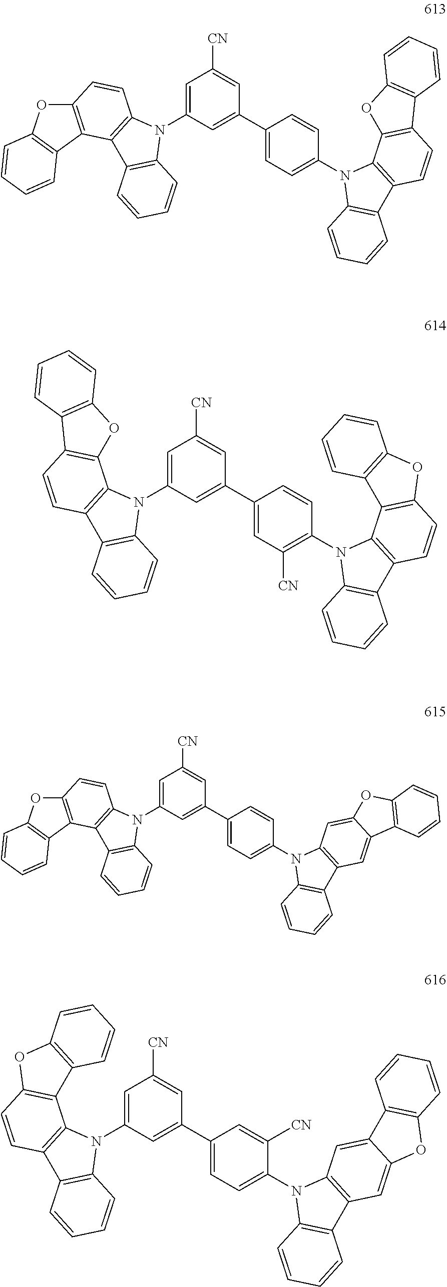 [SMILES: N#Cc1cc(-c2ccc(-n3c4ccccc4c4ccc5c6ccccc6oc5c43)cc2)cc(-n2c3ccccc3c3c4c(ccc32)oc2ccccc24)c1 ; N#Cc1cc(-c2ccc(-n3c4ccccc4c4ccc5oc6ccccc6c5c43)c(C#N)c2)cc(-n2c3ccccc3c3ccc4c5ccccc5oc4c32)c1 ; N#Cc1cc(-c2ccc(-n3c4ccccc4c4cc5c(cc43)oc3ccccc35)cc2)cc(-n2c3ccccc3c3c4c(ccc32)oc2ccccc24)c1 ; N#Cc1cc(-c2ccc(-n3c4ccccc4c4cc5oc6ccccc6c5cc43)c(C#N)c2)cc(-n2c3ccccc3c3ccc4oc5ccccc5c4c32)c1]

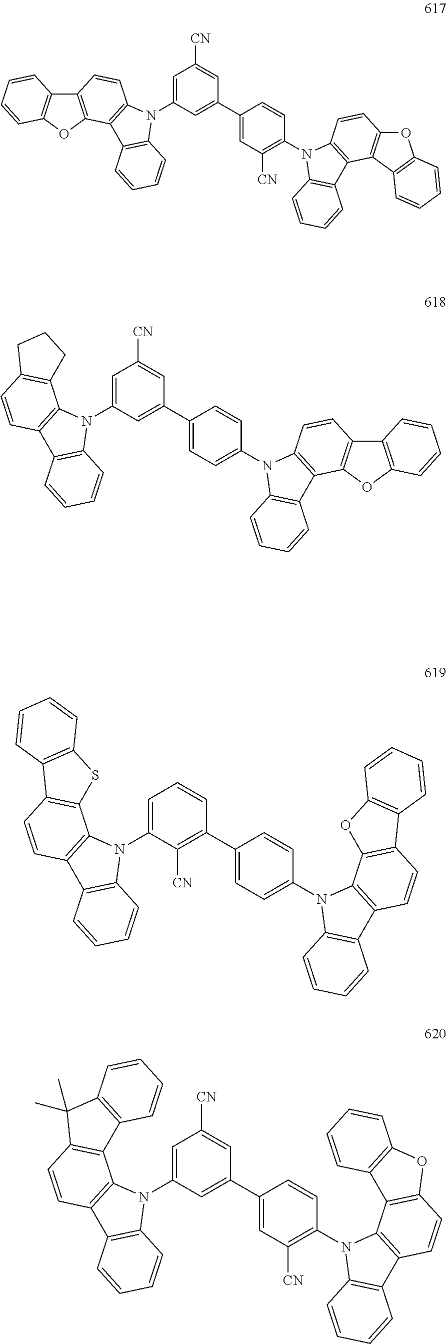 [SMILES: N#Cc1cc(-c2ccc(-n3c4ccccc4c4c5c(ccc43)oc3ccccc35)c(C#N)c2)cc(-n2c3ccccc3c3c4oc5ccccc5c4ccc32)c1 ; N#Cc1cc(-c2ccc(-n3c4ccccc4c4c5oc6ccccc6c5ccc43)cc2)cc(-n2c3ccccc3c3ccc4c(c32)CCC4)c1 ; N#Cc1c(-c2ccc(-n3c4ccccc4c4ccc5c6ccccc6oc5c43)cc2)cccc1-n1c2ccccc2c2ccc3c4ccccc4sc3c21 ; CC1(C)c2ccccc2-c2c1ccc1c3ccccc3n(-c3cc(C#N)cc(-c4ccc(-n5c6ccccc6c6ccc7oc8ccccc8c7c65)c(C#N)c4)c3)c21]

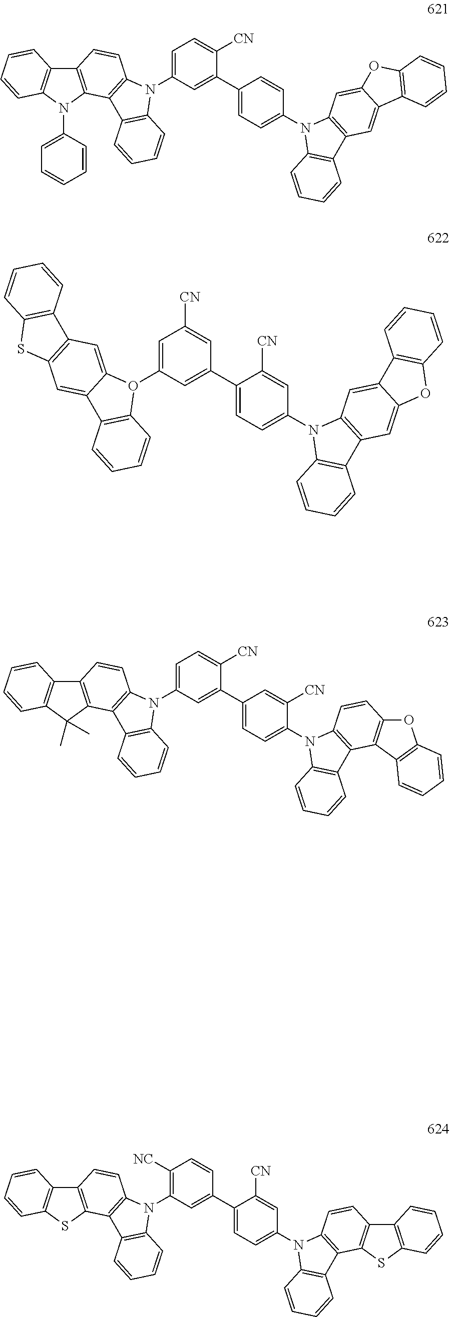

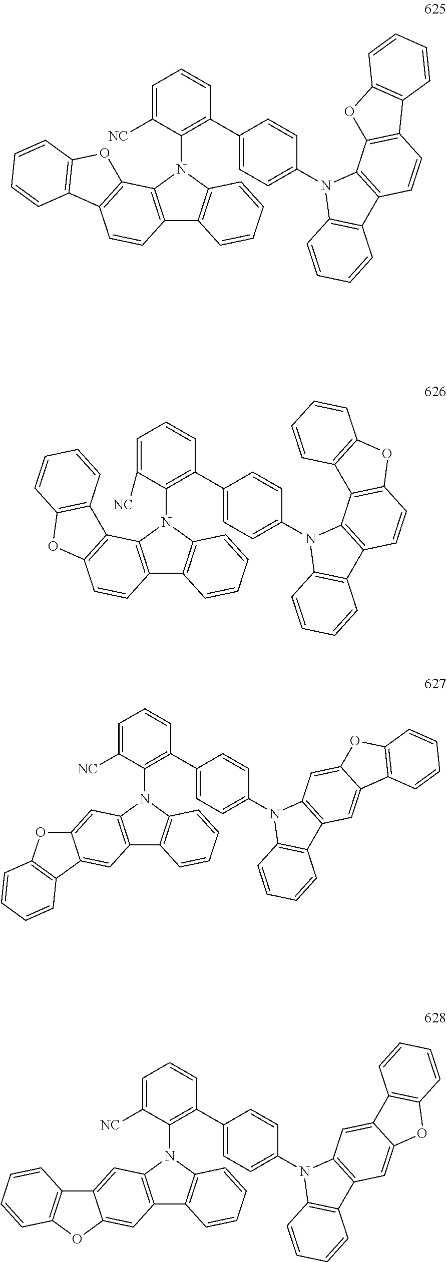 [SMILES: N#Cc1cccc(-c2ccc(-n3c4ccccc4c4ccc5c6ccccc6oc5c43)cc2)c1-n1c2ccccc2c2ccc3c4ccccc4oc3c21 ; N#Cc1cccc(-c2ccc(-n3c4ccccc4c4ccc5oc6ccccc6c5c43)cc2)c1-n1c2ccccc2c2ccc3oc4ccccc4c3c21 ; N#Cc1cccc(-c2ccc(-n3c4ccccc4c4cc5c(cc43)oc3ccccc35)cc2)c1-n1c2ccccc2c2cc3c(cc21)oc1ccccc13 ; N#Cc1cccc(-c2ccc(-n3c4ccccc4c4cc5oc6ccccc6c5cc43)cc2)c1-n1c2ccccc2c2cc3oc4ccccc4c3cc21]

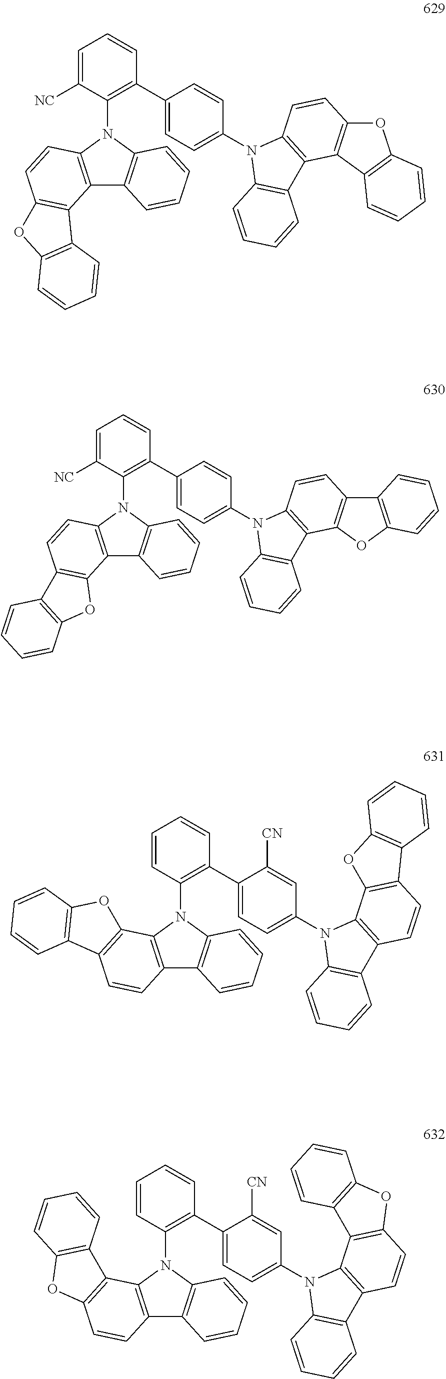 [SMILES: N#Cc1cccc(-c2ccc(-n3c4ccccc4c4c5c(ccc43)oc3ccccc35)cc2)c1-n1c2ccccc2c2c3c(ccc21)oc1ccccc13 ; N#Cc1cccc(-c2ccc(-n3c4ccccc4c4c5oc6ccccc6c5ccc43)cc2)c1-n1c2ccccc2c2c3oc4ccccc4c3ccc21 ; N#Cc1cc(-n2c3ccccc3c3ccc4c5ccccc5oc4c32)ccc1-c1ccccc1-n1c2ccccc2c2ccc3c4ccccc4oc3c21 ; N#Cc1cc(-n2c3ccccc3c3ccc4oc5ccccc5c4c32)ccc1-c1ccccc1-n1c2ccccc2c2ccc3oc4ccccc4c3c21]

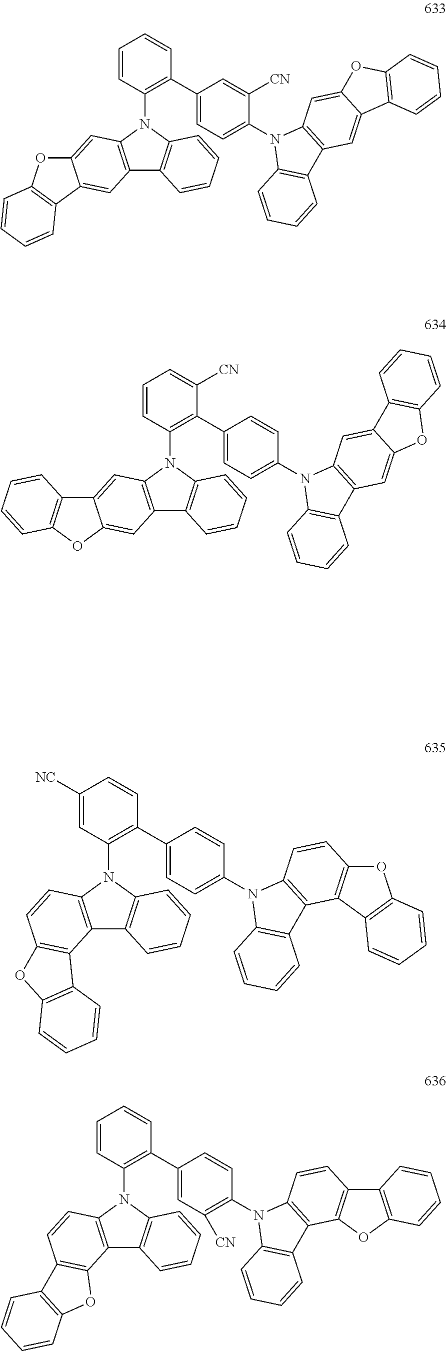 [SMILES: N#Cc1cc(-c2ccccc2-n2c3ccccc3c3cc4c(cc32)oc2ccccc24)ccc1-n1c2ccccc2c2cc3c(cc21)oc1ccccc13 ; N#Cc1cccc(-n2c3ccccc3c3cc4oc5ccccc5c4cc32)c1-c1ccc(-n2c3ccccc3c3cc4oc5ccccc5c4cc32)cc1 ; N#Cc1ccc(-c2ccc(-n3c4ccccc4c4c5c(ccc43)oc3ccccc35)cc2)c(-n2c3ccccc3c3c4c(ccc32)oc2ccccc24)c1 ; N#Cc1cc(-c2ccccc2-n2c3ccccc3c3c4oc5ccccc5c4ccc32)ccc1-n1c2ccccc2c2c3oc4ccccc4c3ccc21]

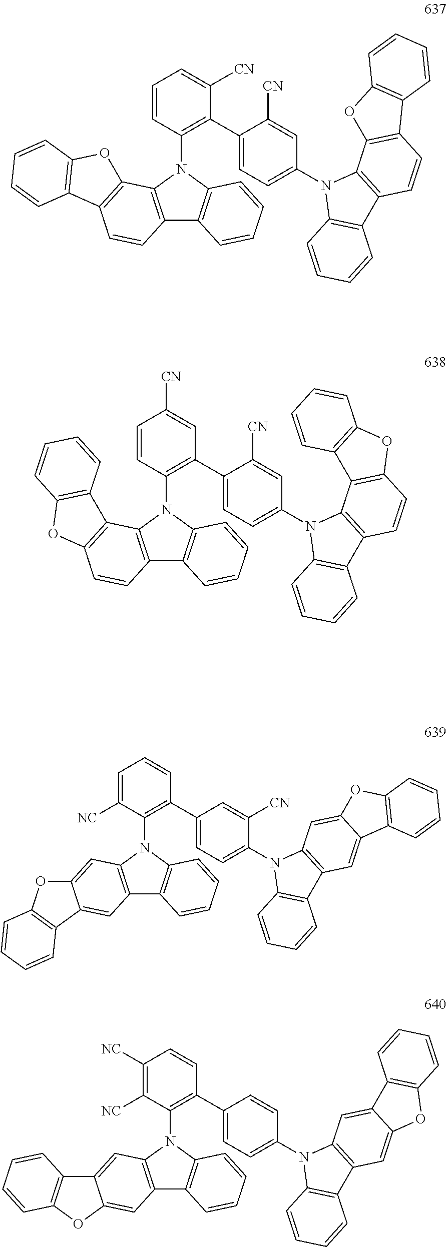 [SMILES: N#Cc1cc(-n2c3ccccc3c3ccc4c5ccccc5oc4c32)ccc1-c1c(C#N)cccc1-n1c2ccccc2c2ccc3c4ccccc4oc3c21 ; N#Cc1ccc(-n2c3ccccc3c3ccc4oc5ccccc5c4c32)c(-c2ccc(-n3c4ccccc4c4ccc5oc6ccccc6c5c43)cc2C#N)c1 ; N#Cc1cc(-c2cccc(C#N)c2-n2c3ccccc3c3cc4c(cc32)oc2ccccc24)ccc1-n1c2ccccc2c2cc3c(cc21)oc1ccccc13 ; N#Cc1ccc(-c2ccc(-n3c4ccccc4c4cc5oc6ccccc6c5cc43)cc2)c(-n2c3ccccc3c3cc4oc5ccccc5c4cc32)c1C#N]

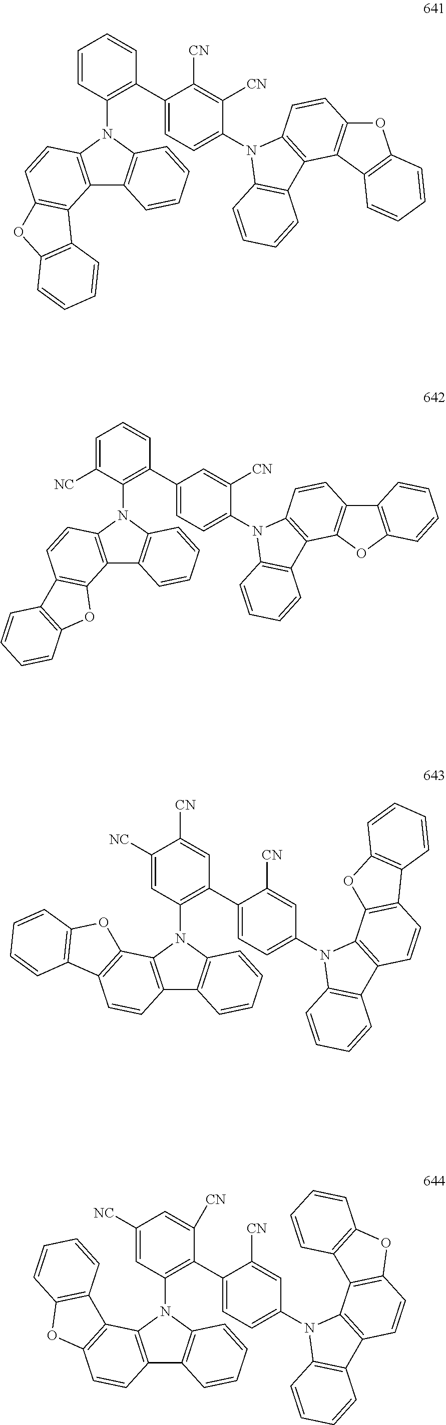 [SMILES: N#Cc1c(-c2ccccc2-n2c3ccccc3c3c4c(ccc32)oc2ccccc24)ccc(-n2c3ccccc3c3c4c(ccc32)oc2ccccc24)c1C#N ; N#Cc1cc(-c2cccc(C#N)c2-n2c3ccccc3c3c4oc5ccccc5c4ccc32)ccc1-n1c2ccccc2c2c3oc4ccccc4c3ccc21 ; N#Cc1cc(-c2ccc(-n3c4ccccc4c4ccc5c6ccccc6oc5c43)cc2C#N)c(-n2c3ccccc3c3ccc4c5ccccc5oc4c32)cc1C#N ; N#Cc1cc(C#N)c(-c2ccc(-n3c4ccccc4c4ccc5oc6ccccc6c5c43)cc2C#N)c(-n2c3ccccc3c3ccc4oc5ccccc5c4c32)c1]

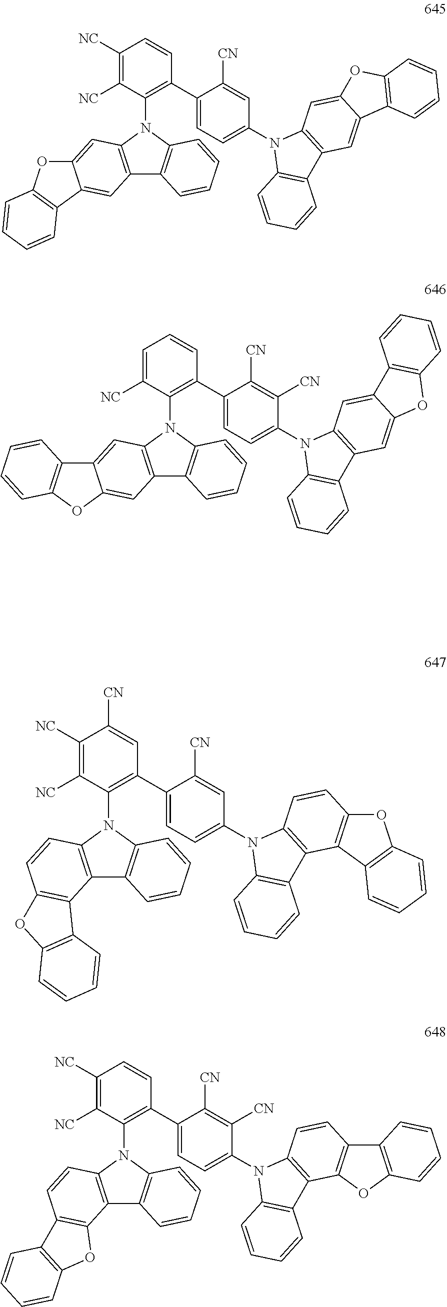 [SMILES: N#Cc1cc(-n2c3ccccc3c3cc4c(cc32)oc2ccccc24)ccc1-c1ccc(C#N)c(C#N)c1-n1c2ccccc2c2cc3c(cc21)oc1ccccc13 ; N#Cc1cccc(-c2ccc(-n3c4ccccc4c4cc5oc6ccccc6c5cc43)c(C#N)c2C#N)c1-n1c2ccccc2c2cc3oc4ccccc4c3cc21 ; N#Cc1cc(-n2c3ccccc3c3c4c(ccc32)oc2ccccc24)ccc1-c1cc(C#N)c(C#N)c(C#N)c1-n1c2ccccc2c2c3c(ccc21)oc1ccccc13 ; N#Cc1ccc(-c2ccc(-n3c4ccccc4c4c5oc6ccccc6c5ccc43)c(C#N)c2C#N)c(-n2c3ccccc3c3c4oc5ccccc5c4ccc32)c1C#N]

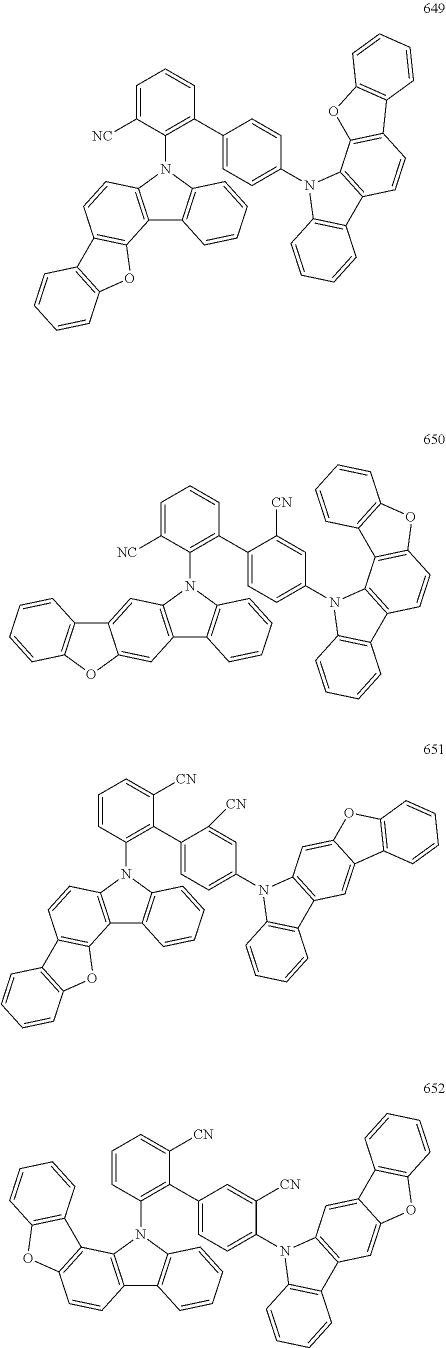 [SMILES: N#Cc1cccc(-c2ccc(-n3c4ccccc4c4ccc5c6ccccc6oc5c43)cc2)c1-n1c2ccccc2c2c3oc4ccccc4c3ccc21 ; N#Cc1cc(-n2c3ccccc3c3ccc4oc5ccccc5c4c32)ccc1-c1cccc(C#N)c1-n1c2ccccc2c2cc3oc4ccccc4c3cc21 ; N#Cc1cc(-n2c3ccccc3c3cc4c(cc32)oc2ccccc24)ccc1-c1c(C#N)cccc1-n1c2ccccc2c2c3oc4ccccc4c3ccc21 ; N#Cc1cc(-c2c(C#N)cccc2-n2c3ccccc3c3ccc4oc5ccccc5c4c32)ccc1-n1c2ccccc2c2cc3oc4ccccc4c3cc21]

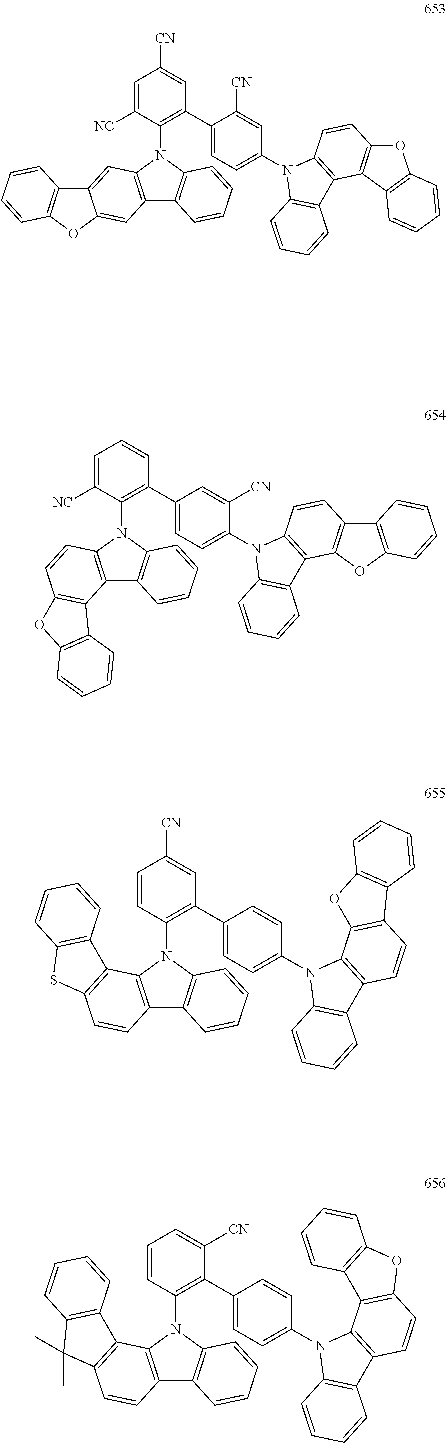 [SMILES: N#Cc1cc(C#N)c(-n2c3ccccc3c3cc4oc5ccccc5c4cc32)c(-c2ccc(-n3c4ccccc4c4c5c(ccc43)oc3ccccc35)cc2C#N)c1 ; N#Cc1cc(-c2cccc(C#N)c2-n2c3ccccc3c3c4c(ccc32)oc2ccccc24)ccc1-n1c2ccccc2c2c3oc4ccccc4c3ccc21 ; N#Cc1ccc(-n2c3ccccc3c3ccc4sc5ccccc5c4c32)c(-c2ccc(-n3c4ccccc4c4ccc5c6ccccc6oc5c43)cc2)c1 ; CC1(C)c2ccccc2-c2c1ccc1c3ccccc3n(-c3cccc(C#N)c3-c3ccc(-n4c5ccccc5c5ccc6oc7ccccc7c6c54)cc3)c21]

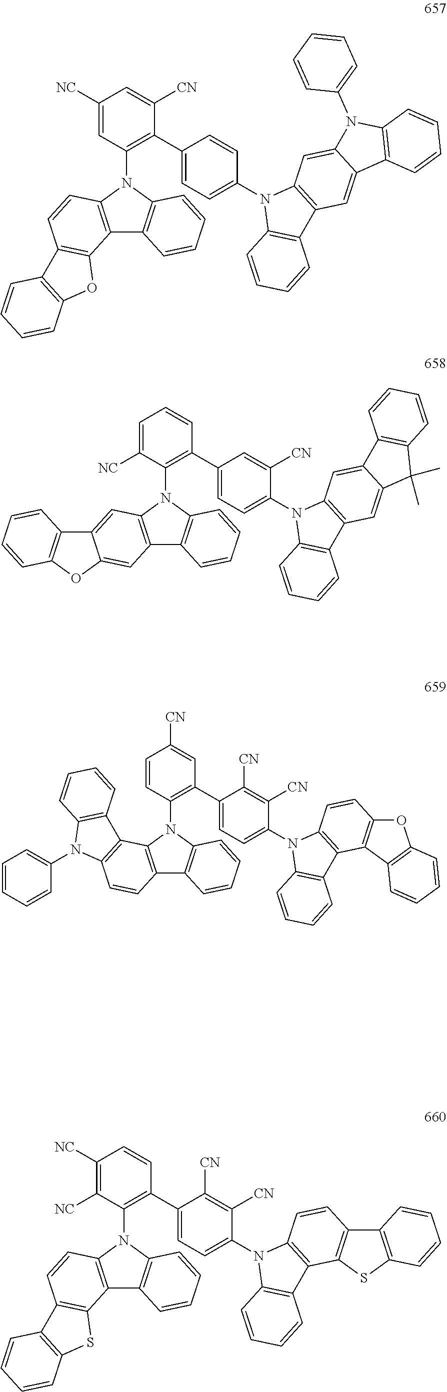 [SMILES: N#Cc1cc(C#N)c(-c2ccc(-n3c4ccccc4c4cc5c6ccccc6n(-c6ccccc6)c5cc43)cc2)c(-n2c3ccccc3c3c4oc5ccccc5c4ccc32)c1 ; CC1(C)c2ccccc2-c2cc3c(cc21)c1ccccc1n3-c1ccc(-c2cccc(C#N)c2-n2c3ccccc3c3cc4oc5ccccc5c4cc32)cc1C#N ; N#Cc1ccc(-n2c3ccccc3c3ccc4c(c5ccccc5n4-c4ccccc4)c32)c(-c2ccc(-n3c4ccccc4c4c5c(ccc43)oc3ccccc35)c(C#N)c2C#N)c1 ; N#Cc1ccc(-c2ccc(-n3c4ccccc4c4c5sc6ccccc6c5ccc43)c(C#N)c2C#N)c(-n2c3ccccc3c3c4sc5ccccc5c4ccc32)c1C#N]

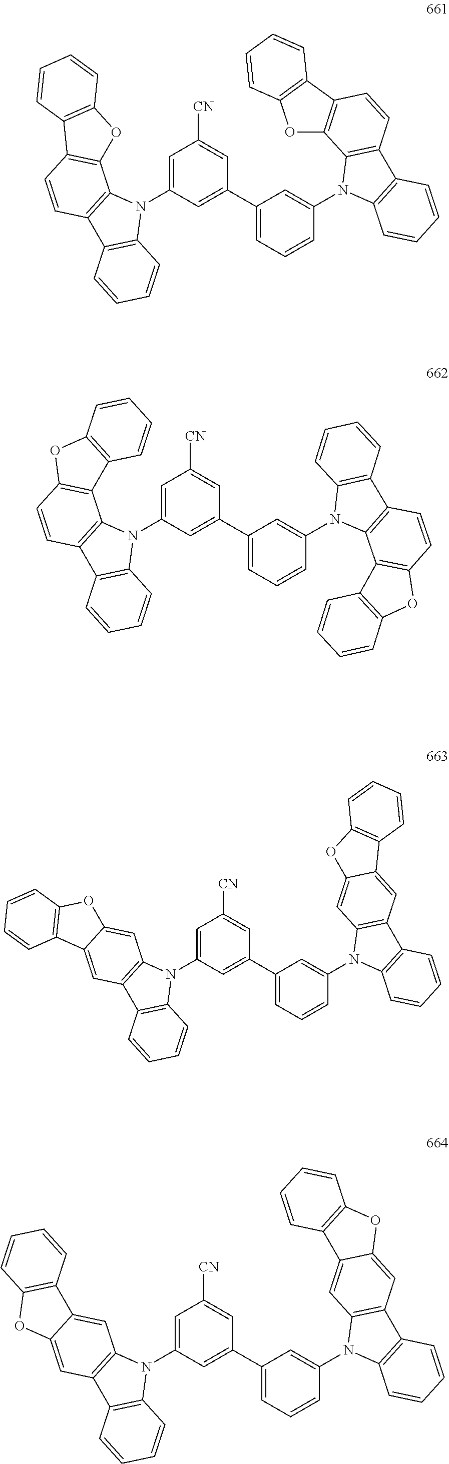 [SMILES: N#Cc1cc(-c2cccc(-n3c4ccccc4c4ccc5c6ccccc6oc5c43)c2)cc(-n2c3ccccc3c3ccc4c5ccccc5oc4c32)c1 ; N#Cc1cc(-c2cccc(-n3c4ccccc4c4ccc5oc6ccccc6c5c43)c2)cc(-n2c3ccccc3c3ccc4oc5ccccc5c4c32)c1 ; N#Cc1cc(-c2cccc(-n3c4ccccc4c4cc5c(cc43)oc3ccccc35)c2)cc(-n2c3ccccc3c3cc4c(cc32)oc2ccccc24)c1 ; N#Cc1cc(-c2cccc(-n3c4ccccc4c4cc5oc6ccccc6c5cc43)c2)cc(-n2c3ccccc3c3cc4oc5ccccc5c4cc32)c1]

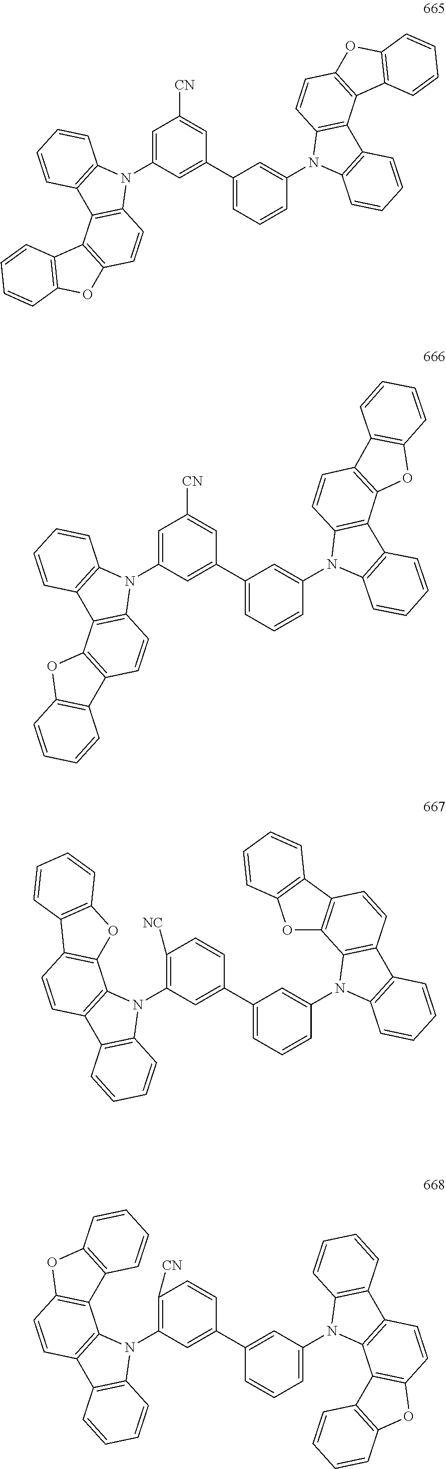 [SMILES: N#Cc1cc(-c2cccc(-n3c4ccccc4c4c5c(ccc43)oc3ccccc35)c2)cc(-n2c3ccccc3c3c4c(ccc32)oc2ccccc24)c1 ; N#Cc1cc(-c2cccc(-n3c4ccccc4c4c5oc6ccccc6c5ccc43)c2)cc(-n2c3ccccc3c3c4oc5ccccc5c4ccc32)c1 ; N#Cc1ccc(-c2cccc(-n3c4ccccc4c4ccc5c6ccccc6oc5c43)c2)cc1-n1c2ccccc2c2ccc3c4ccccc4oc3c21 ; N#Cc1ccc(-c2cccc(-n3c4ccccc4c4ccc5oc6ccccc6c5c43)c2)cc1-n1c2ccccc2c2ccc3oc4ccccc4c3c21]

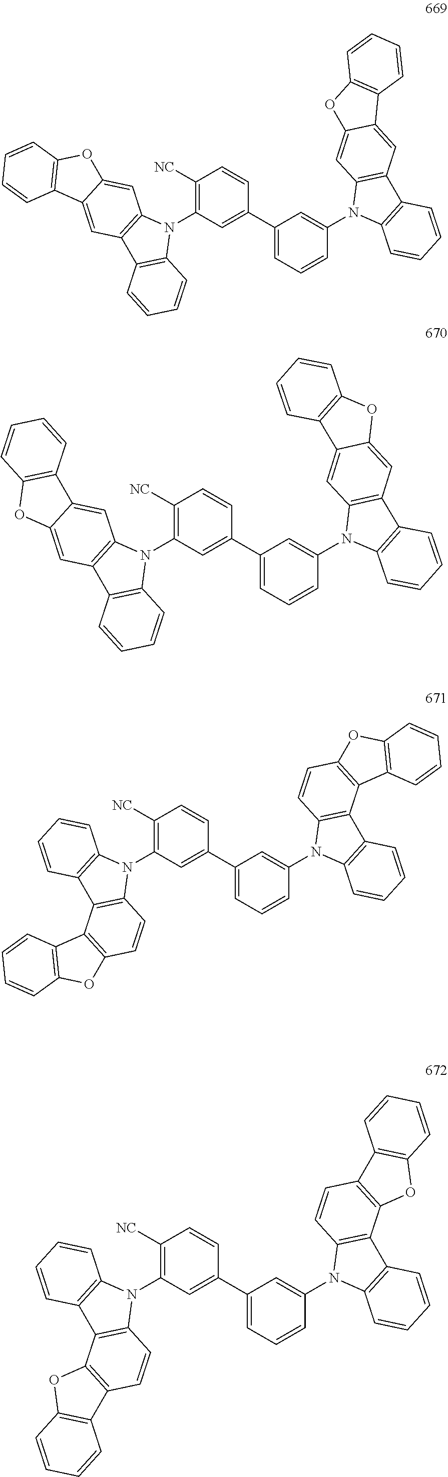 [SMILES: N#Cc1ccc(-c2cccc(-n3c4ccccc4c4cc5c(cc43)oc3ccccc35)c2)cc1-n1c2ccccc2c2cc3c(cc21)oc1ccccc13 ; N#Cc1ccc(-c2cccc(-n3c4ccccc4c4cc5oc6ccccc6c5cc43)c2)cc1-n1c2ccccc2c2cc3oc4ccccc4c3cc21 ; N#Cc1ccc(-c2cccc(-n3c4ccccc4c4c5c(ccc43)oc3ccccc35)c2)cc1-n1c2ccccc2c2c3c(ccc21)oc1ccccc13 ; N#Cc1ccc(-c2cccc(-n3c4ccccc4c4c5oc6ccccc6c5ccc43)c2)cc1-n1c2ccccc2c2c3oc4ccccc4c3ccc21]

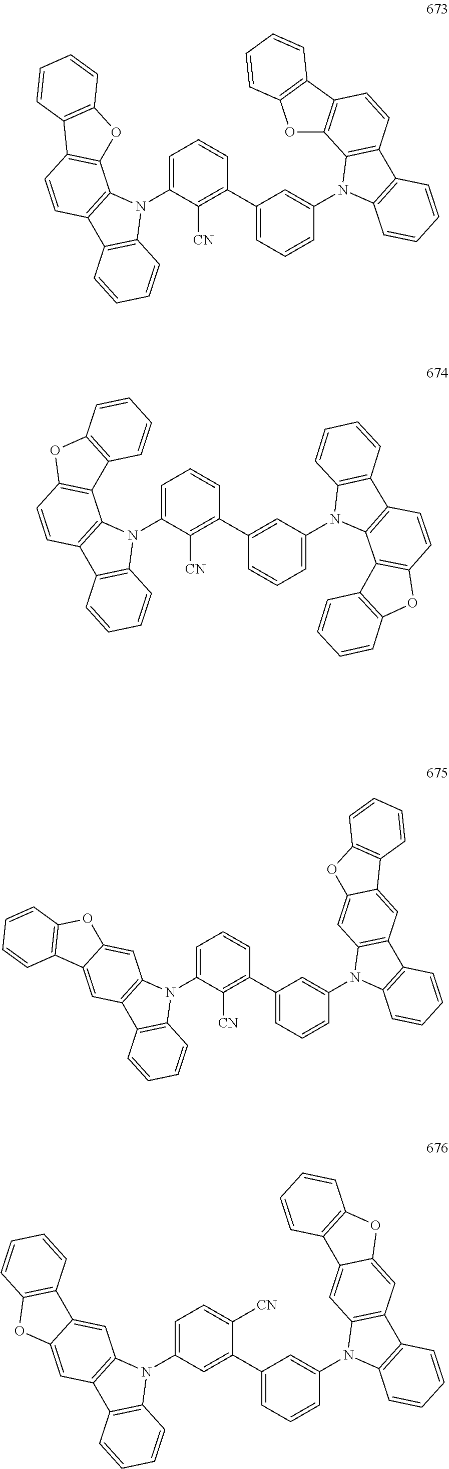 [SMILES: N#Cc1c(-c2cccc(-n3c4ccccc4c4ccc5c6ccccc6oc5c43)c2)cccc1-n1c2ccccc2c2ccc3c4ccccc4oc3c21 ; N#Cc1c(-c2cccc(-n3c4ccccc4c4ccc5oc6ccccc6c5c43)c2)cccc1-n1c2ccccc2c2ccc3oc4ccccc4c3c21 ; N#Cc1c(-c2cccc(-n3c4ccccc4c4cc5c(cc43)oc3ccccc35)c2)cccc1-n1c2ccccc2c2cc3c(cc21)oc1ccccc13 ; N#Cc1ccc(-n2c3ccccc3c3cc4oc5ccccc5c4cc32)cc1-c1cccc(-n2c3ccccc3c3cc4oc5ccccc5c4cc32)c1]

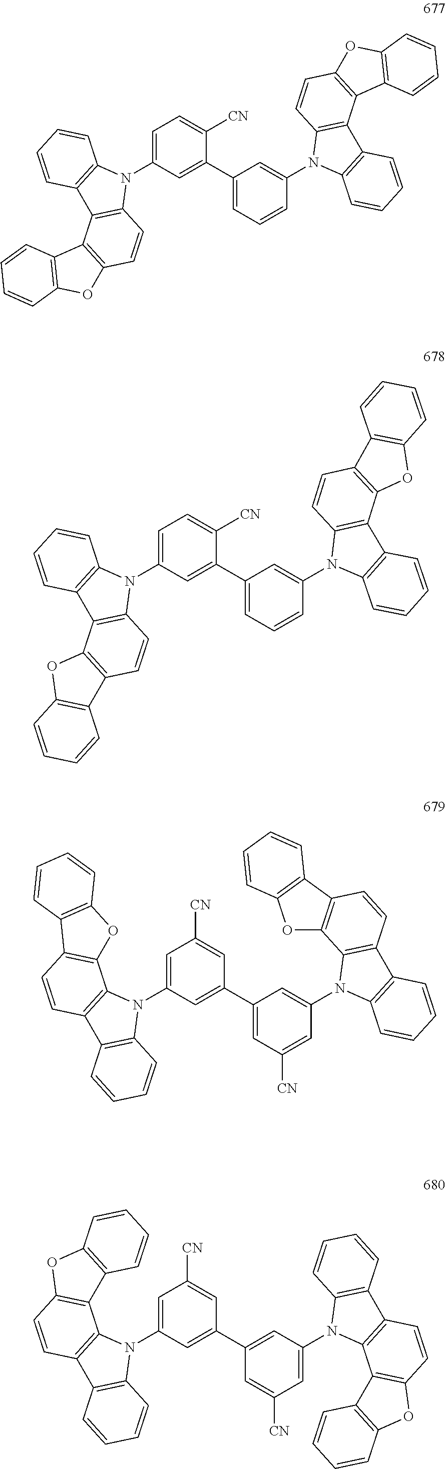 [SMILES: N#Cc1ccc(-n2c3ccccc3c3c4c(ccc32)oc2ccccc24)cc1-c1cccc(-n2c3ccccc3c3c4c(ccc32)oc2ccccc24)c1 ; N#Cc1ccc(-n2c3ccccc3c3c4oc5ccccc5c4ccc32)cc1-c1cccc(-n2c3ccccc3c3c4oc5ccccc5c4ccc32)c1 ; N#Cc1cc(-c2cc(C#N)cc(-n3c4ccccc4c4ccc5c6ccccc6oc5c43)c2)cc(-n2c3ccccc3c3ccc4c5ccccc5oc4c32)c1 ; N#Cc1cc(-c2cc(C#N)cc(-n3c4ccccc4c4ccc5oc6ccccc6c5c43)c2)cc(-n2c3ccccc3c3ccc4oc5ccccc5c4c32)c1]

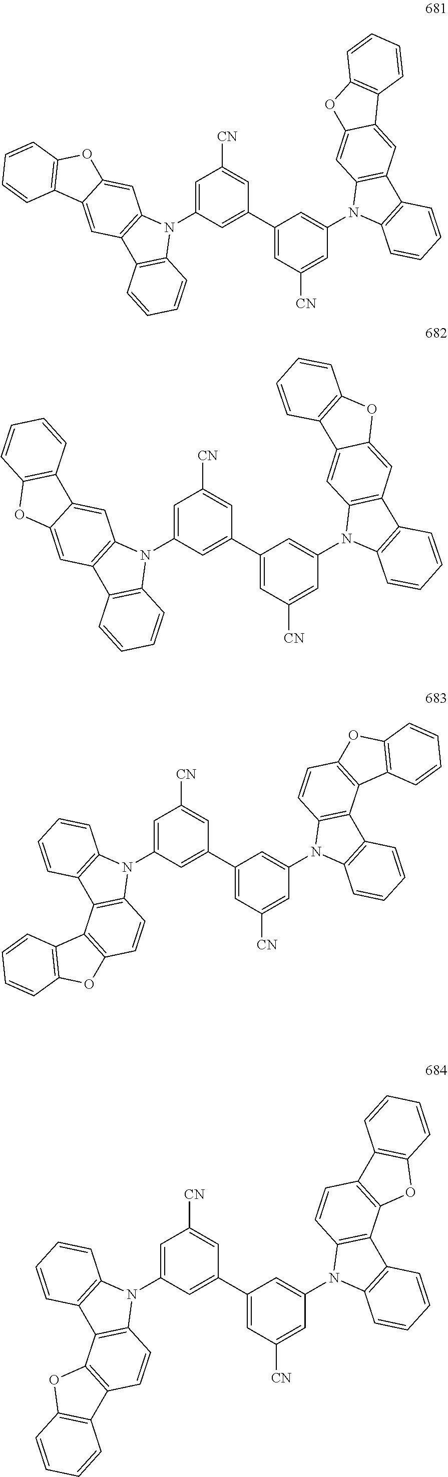 [SMILES: N#Cc1cc(-c2cc(C#N)cc(-n3c4ccccc4c4cc5c(cc43)oc3ccccc35)c2)cc(-n2c3ccccc3c3cc4c(cc32)oc2ccccc24)c1 ; N#Cc1cc(-c2cc(C#N)cc(-n3c4ccccc4c4cc5oc6ccccc6c5cc43)c2)cc(-n2c3ccccc3c3cc4oc5ccccc5c4cc32)c1 ; N#Cc1cc(-c2cc(C#N)cc(-n3c4ccccc4c4c5c(ccc43)oc3ccccc35)c2)cc(-n2c3ccccc3c3c4c(ccc32)oc2ccccc24)c1 ; N#Cc1cc(-c2cc(C#N)cc(-n3c4ccccc4c4c5oc6ccccc6c5ccc43)c2)cc(-n2c3ccccc3c3c4oc5ccccc5c4ccc32)c1]

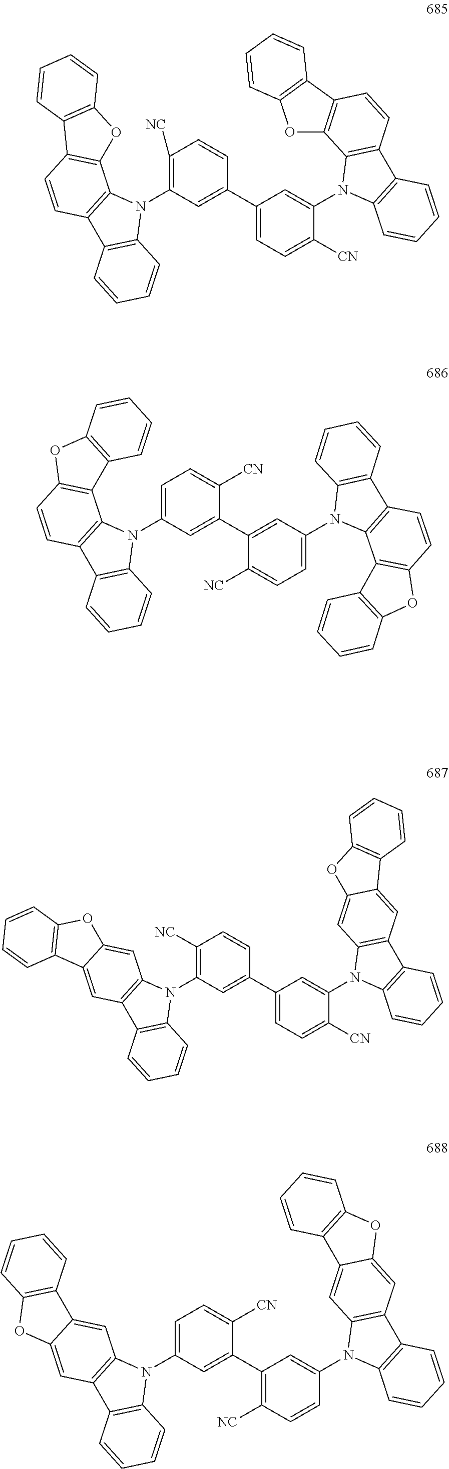 [SMILES: N#Cc1ccc(-c2ccc(C#N)c(-n3c4ccccc4c4ccc5c6ccccc6oc5c43)c2)cc1-n1c2ccccc2c2ccc3c4ccccc4oc3c21 ; N#Cc1ccc(-n2c3ccccc3c3ccc4oc5ccccc5c4c32)cc1-c1cc(-n2c3ccccc3c3ccc4oc5ccccc5c4c32)ccc1C#N ; N#Cc1ccc(-c2ccc(C#N)c(-n3c4ccccc4c4cc5c(cc43)oc3ccccc35)c2)cc1-n1c2ccccc2c2cc3c(cc21)oc1ccccc13 ; N#Cc1ccc(-n2c3ccccc3c3cc4oc5ccccc5c4cc32)cc1-c1cc(-n2c3ccccc3c3cc4oc5ccccc5c4cc32)ccc1C#N]

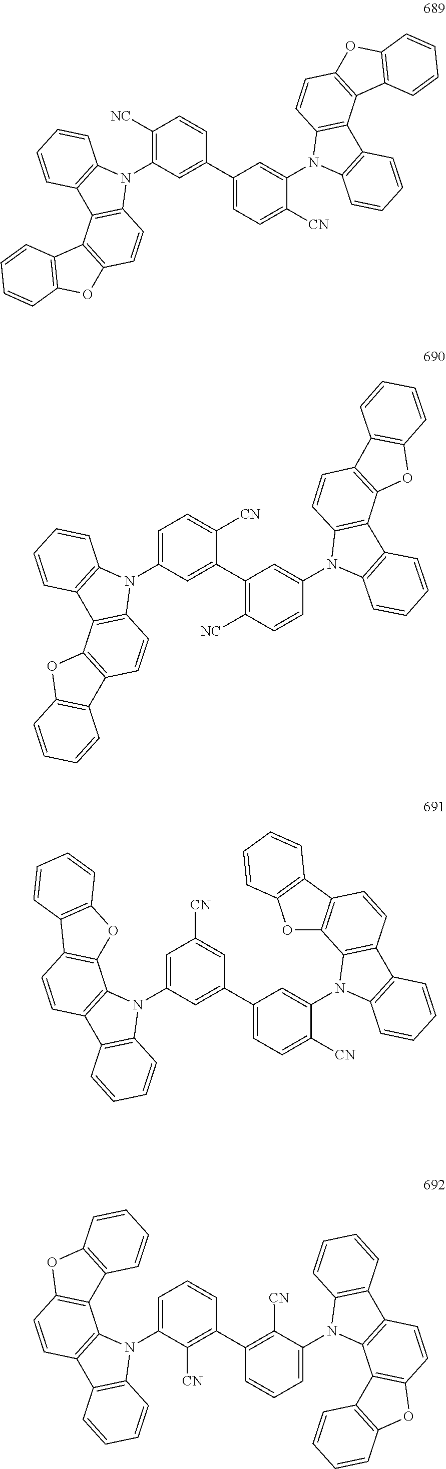 [SMILES: N#Cc1ccc(-c2ccc(C#N)c(-n3c4ccccc4c4c5c(ccc43)oc3ccccc35)c2)cc1-n1c2ccccc2c2c3c(ccc21)oc1ccccc13 ; N#Cc1ccc(-n2c3ccccc3c3c4oc5ccccc5c4ccc32)cc1-c1cc(-n2c3ccccc3c3c4oc5ccccc5c4ccc32)ccc1C#N ; N#Cc1cc(-c2ccc(C#N)c(-n3c4ccccc4c4ccc5c6ccccc6oc5c43)c2)cc(-n2c3ccccc3c3ccc4c5ccccc5oc4c32)c1 ; N#Cc1c(-c2cccc(-n3c4ccccc4c4ccc5oc6ccccc6c5c43)c2C#N)cccc1-n1c2ccccc2c2ccc3oc4ccccc4c3c21]

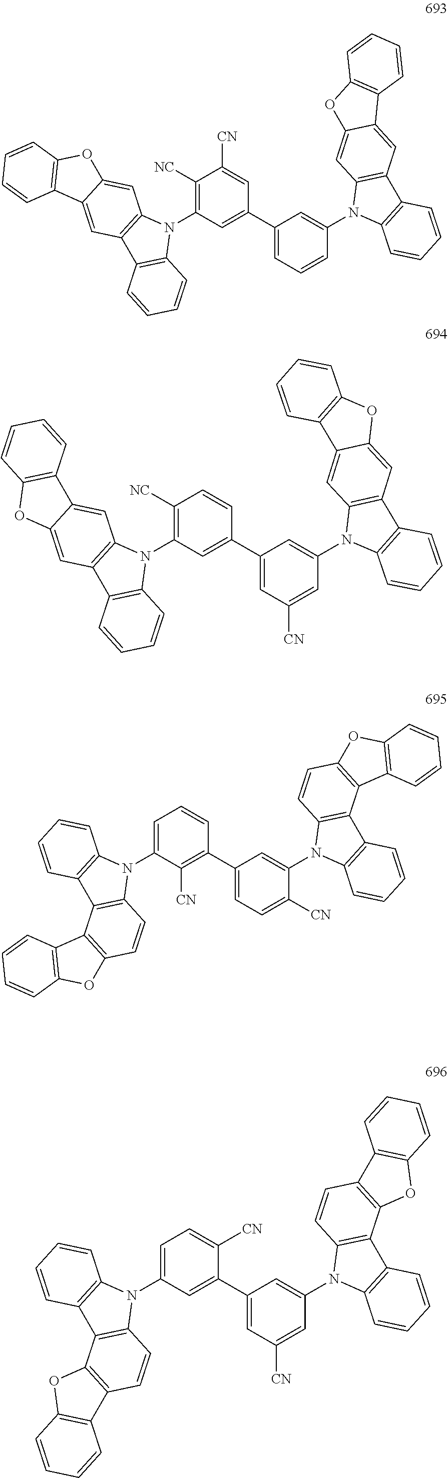 [SMILES: N#Cc1cc(-c2cccc(-n3c4ccccc4c4cc5c(cc43)oc3ccccc35)c2)cc(-n2c3ccccc3c3cc4c(cc32)oc2ccccc24)c1C#N ; N#Cc1cc(-c2ccc(C#N)c(-n3c4ccccc4c4cc5oc6ccccc6c5cc43)c2)cc(-n2c3ccccc3c3cc4oc5ccccc5c4cc32)c1 ; N#Cc1ccc(-c2cccc(-n3c4ccccc4c4c5c(ccc43)oc3ccccc35)c2C#N)cc1-n1c2ccccc2c2c3c(ccc21)oc1ccccc13 ; N#Cc1cc(-c2cc(-n3c4ccccc4c4c5oc6ccccc6c5ccc43)ccc2C#N)cc(-n2c3ccccc3c3c4oc5ccccc5c4ccc32)c1]

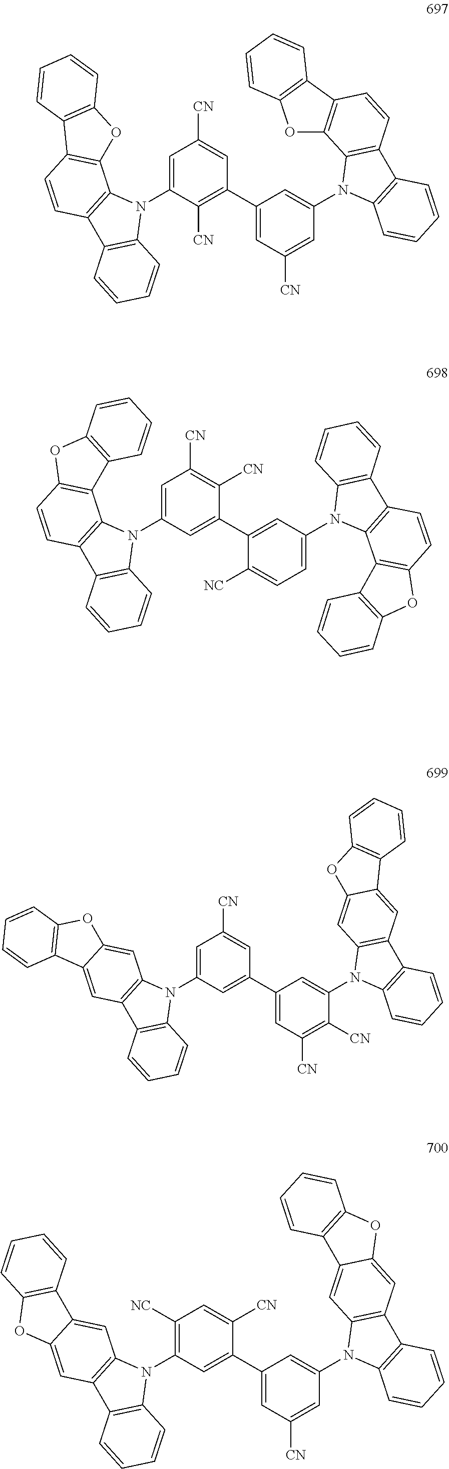 [SMILES: N#Cc1cc(-c2cc(C#N)cc(-n3c4ccccc4c4ccc5c6ccccc6oc5c43)c2C#N)cc(-n2c3ccccc3c3ccc4c5ccccc5oc4c32)c1 ; N#Cc1ccc(-n2c3ccccc3c3ccc4oc5ccccc5c4c32)cc1-c1cc(-n2c3ccccc3c3ccc4oc5ccccc5c4c32)cc(C#N)c1C#N ; N#Cc1cc(-c2cc(C#N)c(C#N)c(-n3c4ccccc4c4cc5c(cc43)oc3ccccc35)c2)cc(-n2c3ccccc3c3cc4c(cc32)oc2ccccc24)c1 ; N#Cc1cc(-c2cc(-n3c4ccccc4c4cc5oc6ccccc6c5cc43)c(C#N)cc2C#N)cc(-n2c3ccccc3c3cc4oc5ccccc5c4cc32)c1]

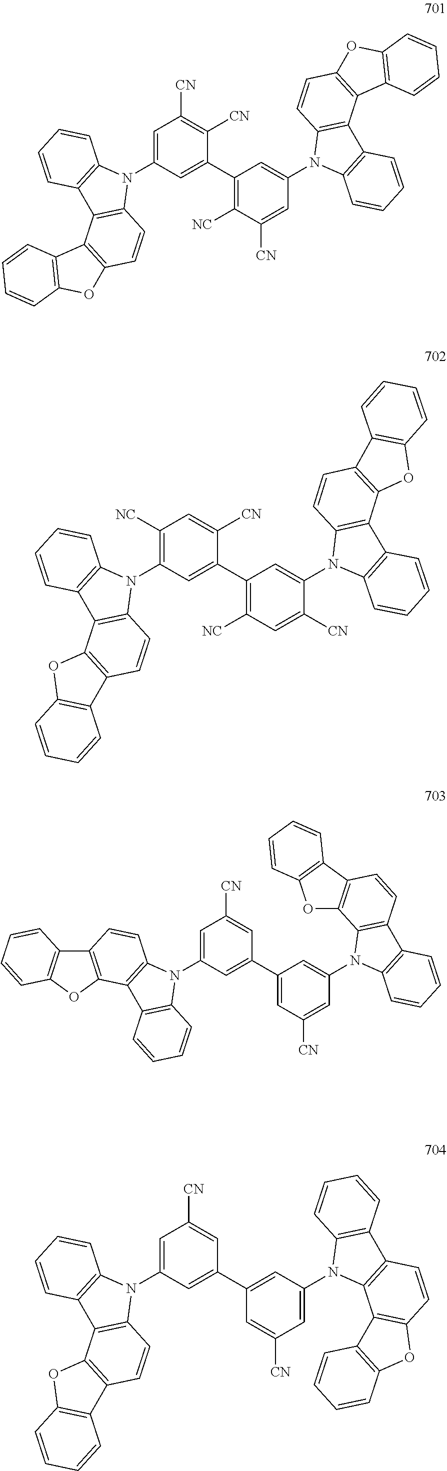 [SMILES: N#Cc1cc(-n2c3ccccc3c3c4c(ccc32)oc2ccccc24)cc(-c2cc(-n3c4ccccc4c4c5c(ccc43)oc3ccccc35)cc(C#N)c2C#N)c1C#N ; N#Cc1cc(C#N)c(-n2c3ccccc3c3c4oc5ccccc5c4ccc32)cc1-c1cc(-n2c3ccccc3c3c4oc5ccccc5c4ccc32)c(C#N)cc1C#N ; N#Cc1cc(-c2cc(C#N)cc(-n3c4ccccc4c4ccc5c6ccccc6oc5c43)c2)cc(-n2c3ccccc3c3c4oc5ccccc5c4ccc32)c1 ; N#Cc1cc(-c2cc(C#N)cc(-n3c4ccccc4c4ccc5oc6ccccc6c5c43)c2)cc(-n2c3ccccc3c3c4oc5ccccc5c4ccc32)c1]

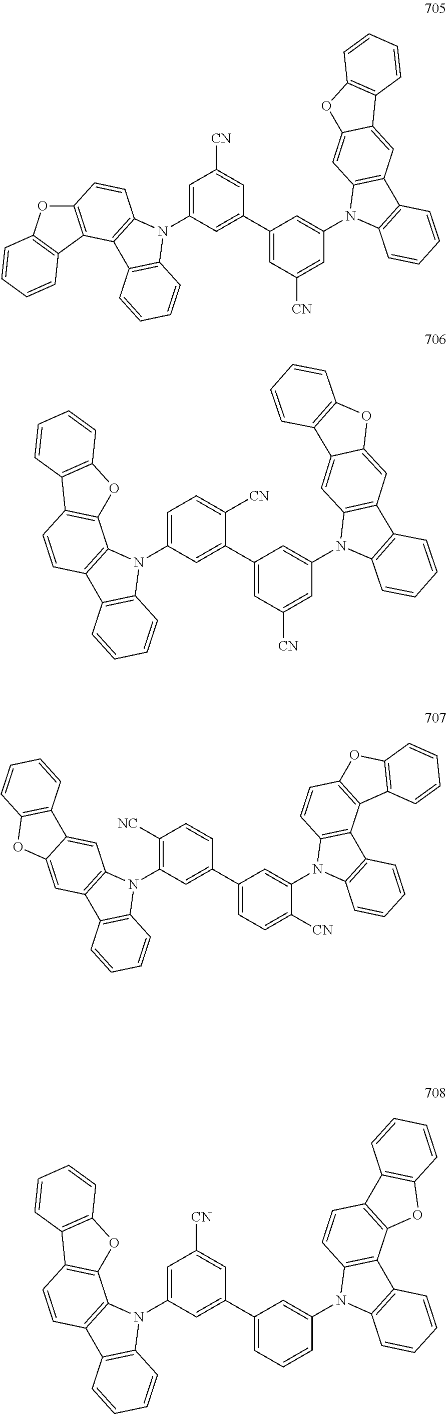 [SMILES: N#Cc1cc(-c2cc(C#N)cc(-n3c4ccccc4c4c5c(ccc43)oc3ccccc35)c2)cc(-n2c3ccccc3c3cc4c(cc32)oc2ccccc24)c1 ; N#Cc1cc(-c2cc(-n3c4ccccc4c4ccc5c6ccccc6oc5c43)ccc2C#N)cc(-n2c3ccccc3c3cc4oc5ccccc5c4cc32)c1 ; N#Cc1ccc(-c2ccc(C#N)c(-n3c4ccccc4c4c5c(ccc43)oc3ccccc35)c2)cc1-n1c2ccccc2c2cc3oc4ccccc4c3cc21 ; N#Cc1cc(-c2cccc(-n3c4ccccc4c4c5oc6ccccc6c5ccc43)c2)cc(-n2c3ccccc3c3ccc4c5ccccc5oc4c32)c1]

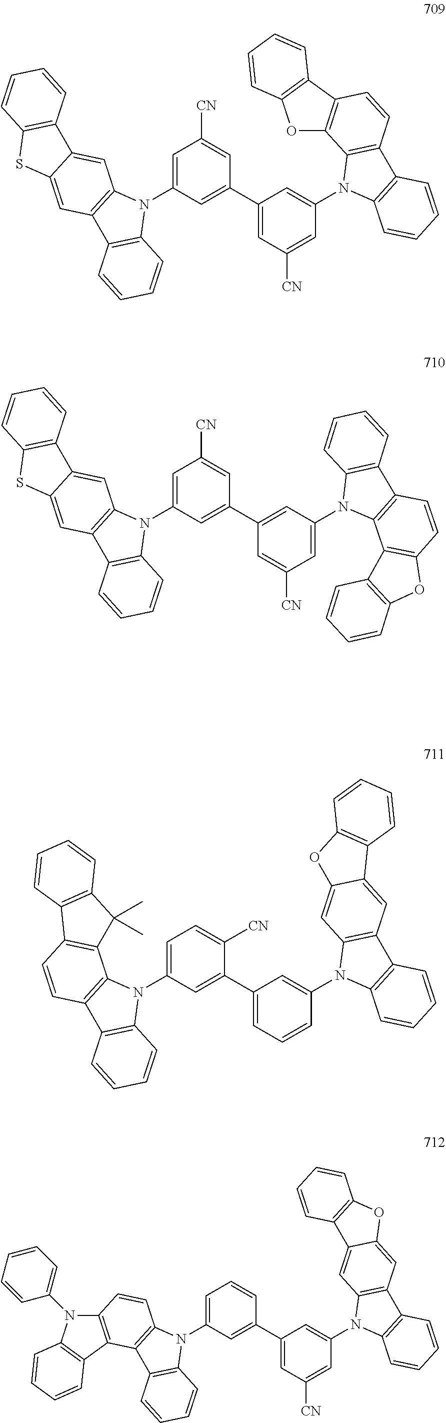 [SMILES: N#Cc1cc(-c2cc(C#N)cc(-n3c4ccccc4c4ccc5c6ccccc6oc5c43)c2)cc(-n2c3ccccc3c3cc4sc5ccccc5c4cc32)c1 ; N#Cc1cc(-c2cc(C#N)cc(-n3c4ccccc4c4ccc5oc6ccccc6c5c43)c2)cc(-n2c3ccccc3c3cc4sc5ccccc5c4cc32)c1 ; CC1(C)c2ccccc2-c2ccc3c4ccccc4n(-c4ccc(C#N)c(-c5cccc(-n6c7ccccc7c7cc8c(cc76)oc6ccccc68)c5)c4)c3c21 ; N#Cc1cc(-c2cccc(-n3c4ccccc4c4c5c6ccccc6n(-c6ccccc6)c5ccc43)c2)cc(-n2c3ccccc3c3cc4oc5ccccc5c4cc32)c1]

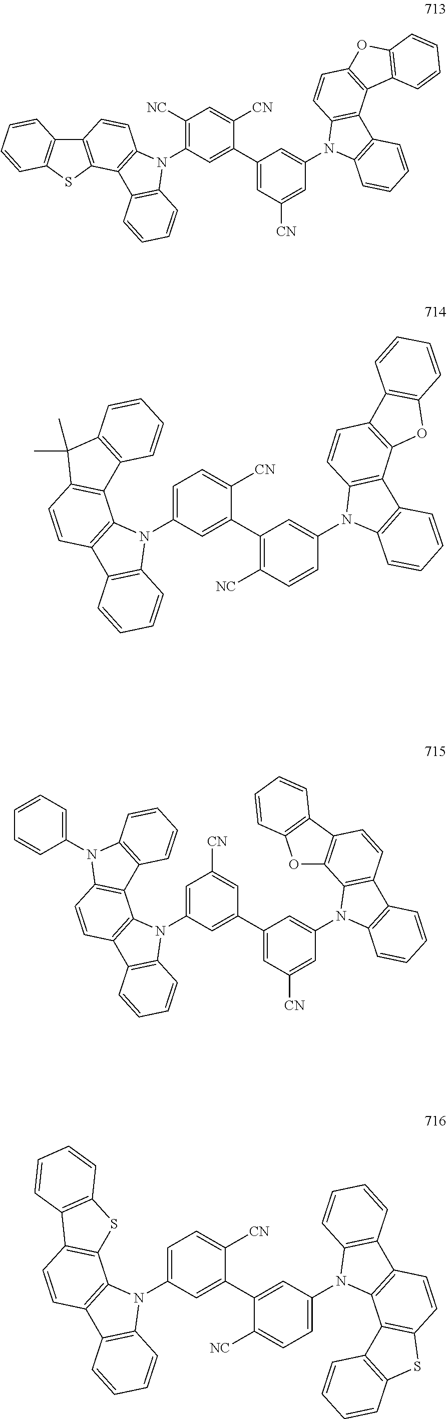 [SMILES: N#Cc1cc(-c2cc(-n3c4ccccc4c4c5sc6ccccc6c5ccc43)c(C#N)cc2C#N)cc(-n2c3ccccc3c3c4c(ccc32)oc2ccccc24)c1 ; CC1(C)c2ccccc2-c2c1ccc1c3ccccc3n(-c3ccc(C#N)c(-c4cc(-n5c6ccccc6c6c7oc8ccccc8c7ccc65)ccc4C#N)c3)c21 ; N#Cc1cc(-c2cc(C#N)cc(-n3c4ccccc4c4ccc5c(c6ccccc6n5-c5ccccc5)c43)c2)cc(-n2c3ccccc3c3ccc4c5ccccc5oc4c32)c1 ; N#Cc1ccc(-n2c3ccccc3c3ccc4c5ccccc5sc4c32)cc1-c1cc(-n2c3ccccc3c3ccc4sc5ccccc5c4c32)ccc1C#N]

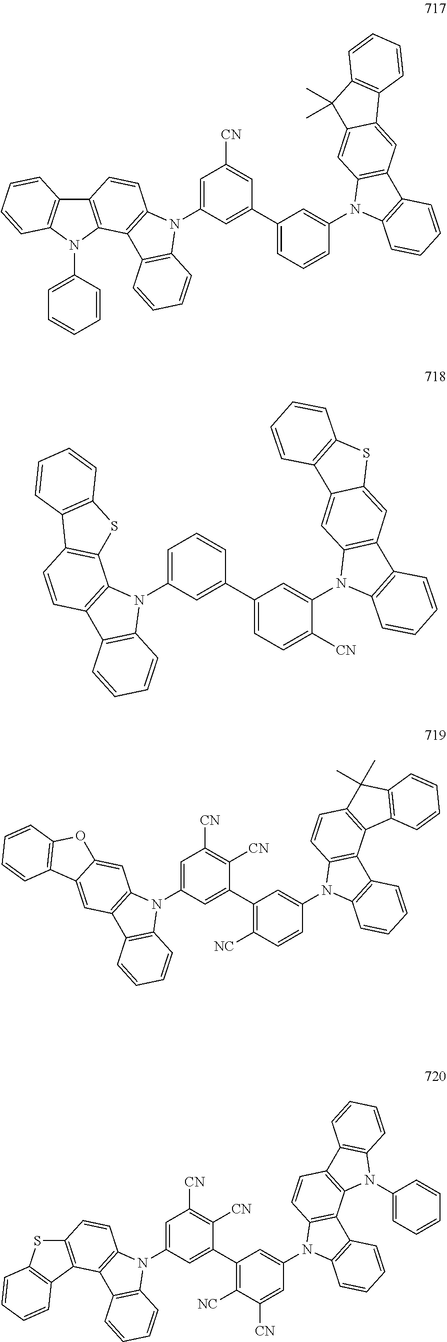 [SMILES: CC1(C)c2ccccc2-c2cc3c4ccccc4n(-c4cccc(-c5cc(C#N)cc(-n6c7ccccc7c7c6ccc6c8ccccc8n(-c8ccccc8)c67)c5)c4)c3cc21 ; N#Cc1ccc(-c2cccc(-n3c4ccccc4c4ccc5c6ccccc6sc5c43)c2)cc1-n1c2ccccc2c2cc3sc4ccccc4c3cc21 ; CC1(C)c2ccccc2-c2c1ccc1c2c2ccccc2n1-c1ccc(C#N)c(-c2cc(-n3c4ccccc4c4cc5c(cc43)oc3ccccc35)cc(C#N)c2C#N)c1 ; N#Cc1cc(-n2c3ccccc3c3c4c(ccc32)sc2ccccc24)cc(-c2cc(-n3c4ccccc4c4c3ccc3c5ccccc5n(-c5ccccc5)c34)cc(C#N)c2C#N)c1C#N]

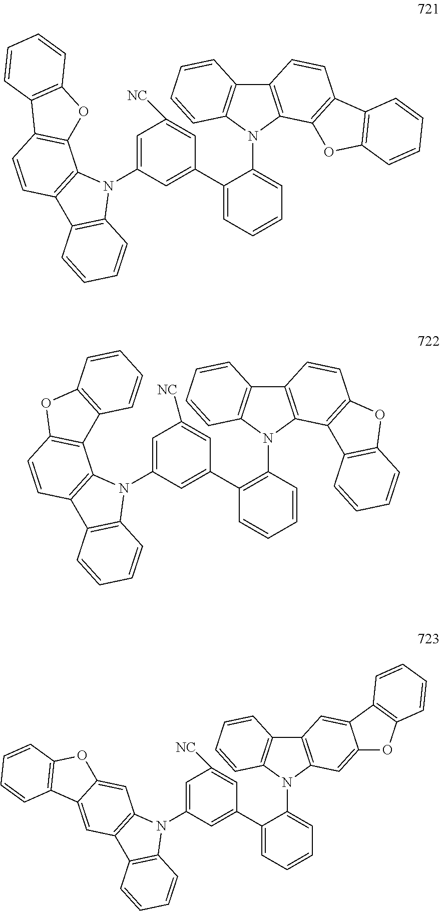 [SMILES: N#Cc1cc(-c2ccccc2-n2c3ccccc3c3ccc4c5ccccc5oc4c32)cc(-n2c3ccccc3c3ccc4c5ccccc5oc4c32)c1 ; N#Cc1cc(-c2ccccc2-n2c3ccccc3c3ccc4oc5ccccc5c4c32)cc(-n2c3ccccc3c3ccc4oc5ccccc5c4c32)c1 ; N#Cc1cc(-c2ccccc2-n2c3ccccc3c3cc4c(cc32)oc2ccccc24)cc(-n2c3ccccc3c3cc4c(cc32)oc2ccccc24)c1]

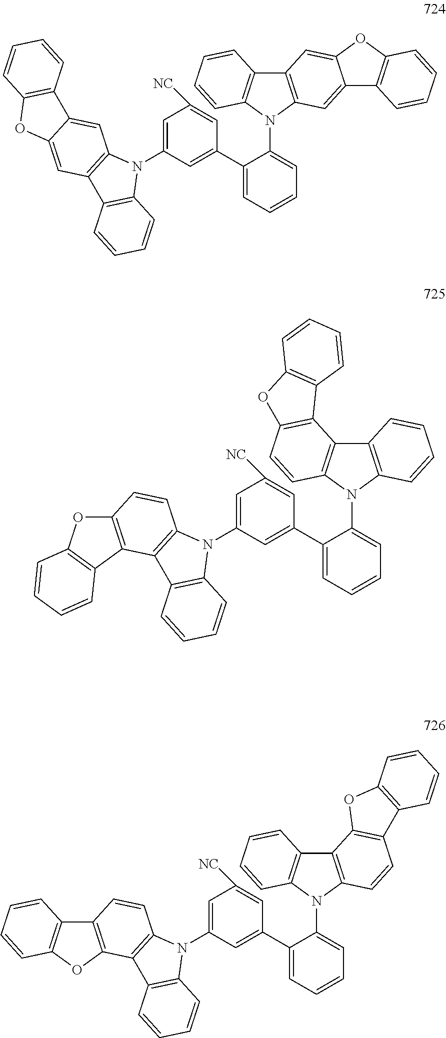 [SMILES: N#Cc1cc(-c2ccccc2-n2c3ccccc3c3cc4oc5ccccc5c4cc32)cc(-n2c3ccccc3c3cc4oc5ccccc5c4cc32)c1 ; N#Cc1cc(-c2ccccc2-n2c3ccccc3c3c4c(ccc32)oc2ccccc24)cc(-n2c3ccccc3c3c4c(ccc32)oc2ccccc24)c1 ; N#Cc1cc(-c2ccccc2-n2c3ccccc3c3c4oc5ccccc5c4ccc32)cc(-n2c3ccccc3c3c4oc5ccccc5c4ccc32)c1]

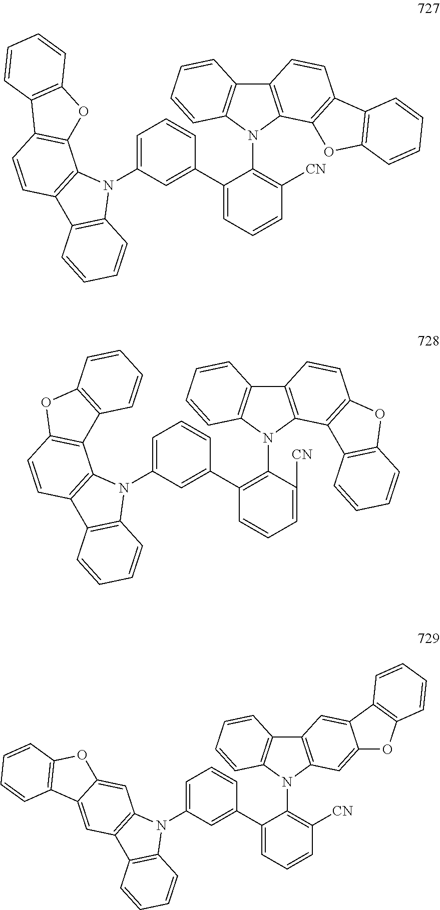 [SMILES: N#Cc1cccc(-c2cccc(-n3c4ccccc4c4ccc5c6ccccc6oc5c43)c2)c1-n1c2ccccc2c2ccc3c4ccccc4oc3c21 ; N#Cc1cccc(-c2cccc(-n3c4ccccc4c4ccc5oc6ccccc6c5c43)c2)c1-n1c2ccccc2c2ccc3oc4ccccc4c3c21 ; N#Cc1cccc(-c2cccc(-n3c4ccccc4c4cc5c(cc43)oc3ccccc35)c2)c1-n1c2ccccc2c2cc3c(cc21)oc1ccccc13]

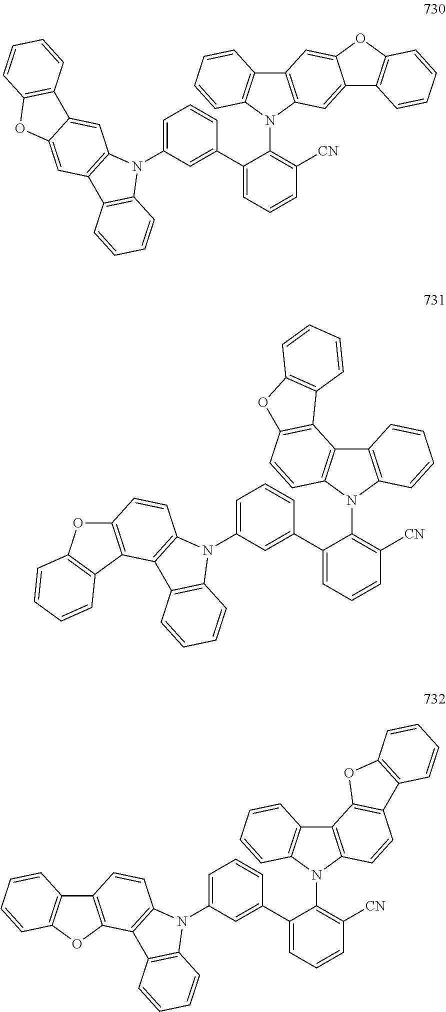 [SMILES: N#Cc1cccc(-c2cccc(-n3c4ccccc4c4cc5oc6ccccc6c5cc43)c2)c1-n1c2ccccc2c2cc3oc4ccccc4c3cc21 ; N#Cc1cccc(-c2cccc(-n3c4ccccc4c4c5c(ccc43)oc3ccccc35)c2)c1-n1c2ccccc2c2c3c(ccc21)oc1ccccc13 ; N#Cc1cccc(-c2cccc(-n3c4ccccc4c4c5oc6ccccc6c5ccc43)c2)c1-n1c2ccccc2c2c3oc4ccccc4c3ccc21]

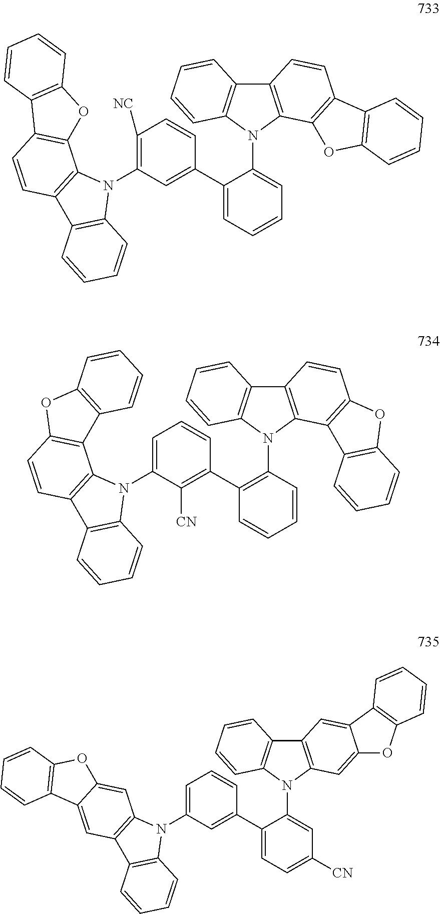 [SMILES: N#Cc1ccc(-c2ccccc2-n2c3ccccc3c3ccc4c5ccccc5oc4c32)cc1-n1c2ccccc2c2ccc3c4ccccc4oc3c21 ; N#Cc1c(-c2ccccc2-n2c3ccccc3c3ccc4oc5ccccc5c4c32)cccc1-n1c2ccccc2c2ccc3oc4ccccc4c3c21 ; N#Cc1ccc(-c2cccc(-n3c4ccccc4c4cc5c(cc43)oc3ccccc35)c2)c(-n2c3ccccc3c3cc4c(cc32)oc2ccccc24)c1]

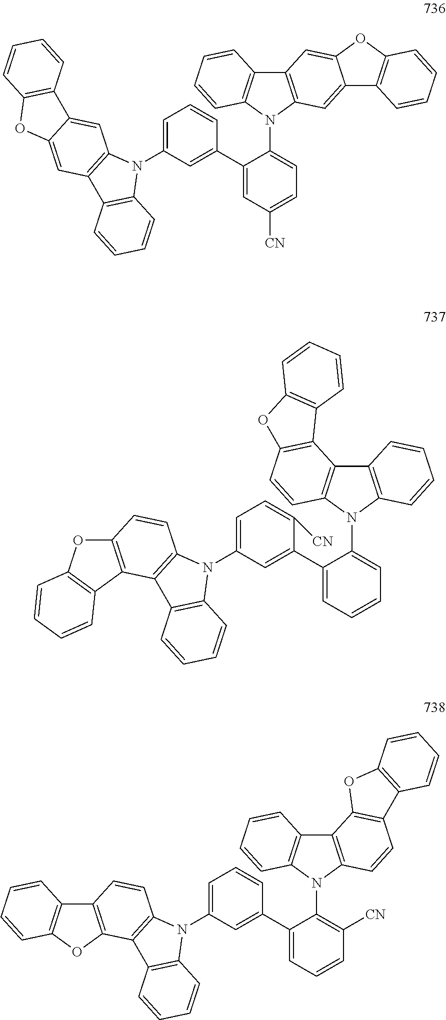 [SMILES: N#Cc1ccc(-n2c3ccccc3c3cc4oc5ccccc5c4cc32)c(-c2cccc(-n3c4ccccc4c4cc5oc6ccccc6c5cc43)c2)c1 ; N#Cc1ccc(-n2c3ccccc3c3c4c(ccc32)oc2ccccc24)cc1-c1ccccc1-n1c2ccccc2c2c3c(ccc21)oc1ccccc13 ; N#Cc1cccc(-c2cccc(-n3c4ccccc4c4c5oc6ccccc6c5ccc43)c2)c1-n1c2ccccc2c2c3oc4ccccc4c3ccc21]

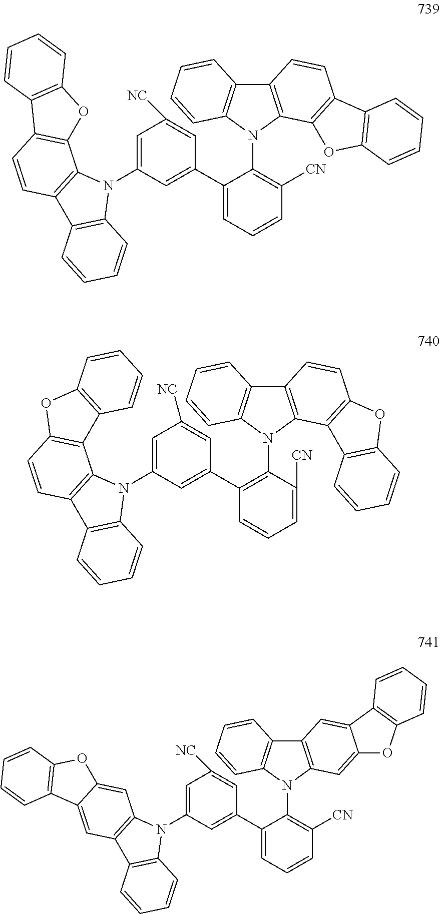 [SMILES: N#Cc1cc(-c2cccc(C#N)c2-n2c3ccccc3c3ccc4c5ccccc5oc4c32)cc(-n2c3ccccc3c3ccc4c5ccccc5oc4c32)c1 ; N#Cc1cc(-c2cccc(C#N)c2-n2c3ccccc3c3ccc4oc5ccccc5c4c32)cc(-n2c3ccccc3c3ccc4oc5ccccc5c4c32)c1 ; N#Cc1cc(-c2cccc(C#N)c2-n2c3ccccc3c3cc4c(cc32)oc2ccccc24)cc(-n2c3ccccc3c3cc4c(cc32)oc2ccccc24)c1]

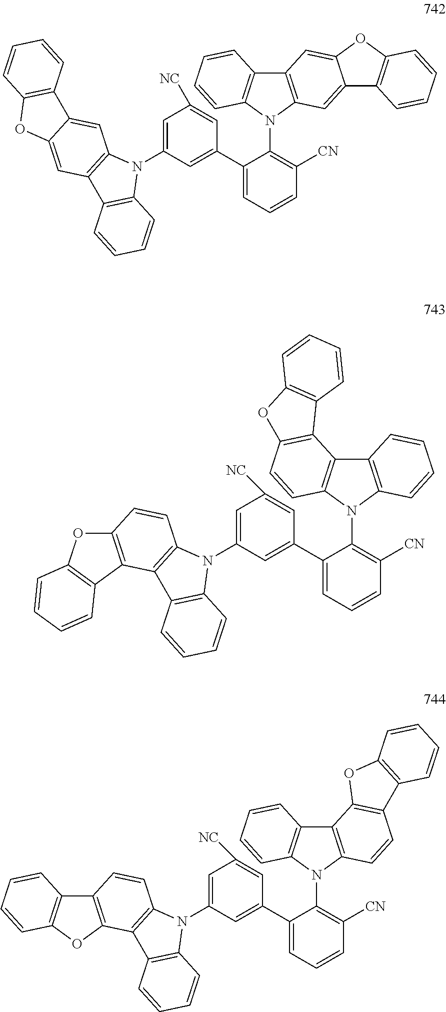 [SMILES: N#Cc1cc(-c2cccc(C#N)c2-n2c3ccccc3c3cc4oc5ccccc5c4cc32)cc(-n2c3ccccc3c3cc4oc5ccccc5c4cc32)c1 ; N#Cc1cc(-c2cccc(C#N)c2-n2c3ccccc3c3c4c(ccc32)oc2ccccc24)cc(-n2c3ccccc3c3c4c(ccc32)oc2ccccc24)c1 ; N#Cc1cc(-c2cccc(C#N)c2-n2c3ccccc3c3c4oc5ccccc5c4ccc32)cc(-n2c3ccccc3c3c4oc5ccccc5c4ccc32)c1]

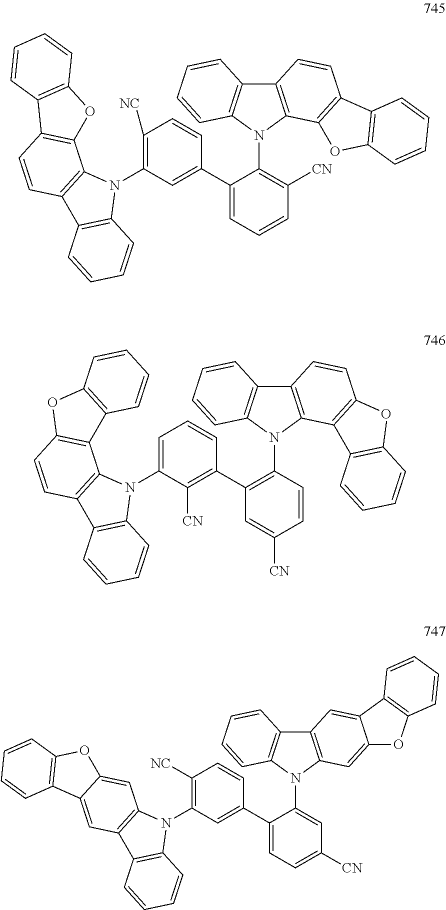 [SMILES: N#Cc1ccc(-c2cccc(C#N)c2-n2c3ccccc3c3ccc4c5ccccc5oc4c32)cc1-n1c2ccccc2c2ccc3c4ccccc4oc3c21 ; N#Cc1ccc(-n2c3ccccc3c3ccc4oc5ccccc5c4c32)c(-c2cccc(-n3c4ccccc4c4ccc5oc6ccccc6c5c43)c2C#N)c1 ; N#Cc1ccc(-c2ccc(C#N)c(-n3c4ccccc4c4cc5c(cc43)oc3ccccc35)c2)c(-n2c3ccccc3c3cc4c(cc32)oc2ccccc24)c1]

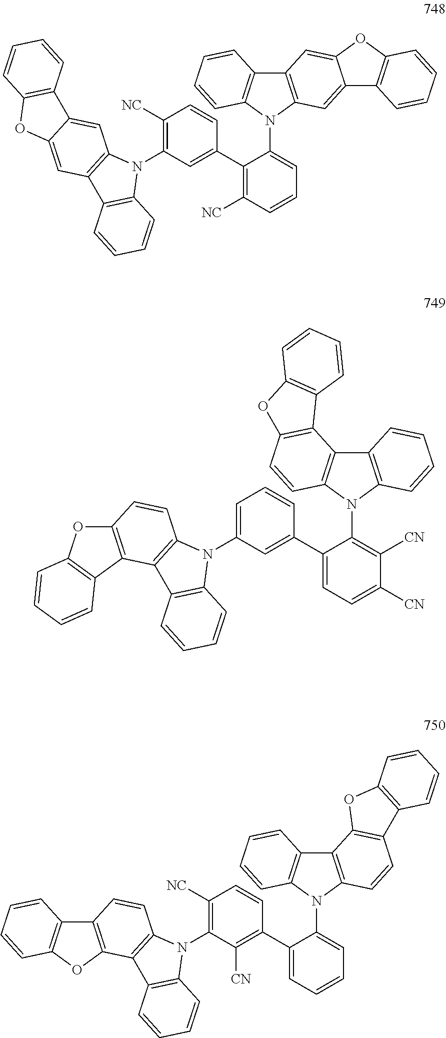 [SMILES: N#Cc1ccc(-c2c(C#N)cccc2-n2c3ccccc3c3cc4oc5ccccc5c4cc32)cc1-n1c2ccccc2c2cc3oc4ccccc4c3cc21 ; N#Cc1ccc(-c2cccc(-n3c4ccccc4c4c5c(ccc43)oc3ccccc35)c2)c(-n2c3ccccc3c3c4c(ccc32)oc2ccccc24)c1C#N ; N#Cc1ccc(-c2ccccc2-n2c3ccccc3c3c4oc5ccccc5c4ccc32)c(C#N)c1-n1c2ccccc2c2c3oc4ccccc4c3ccc21]

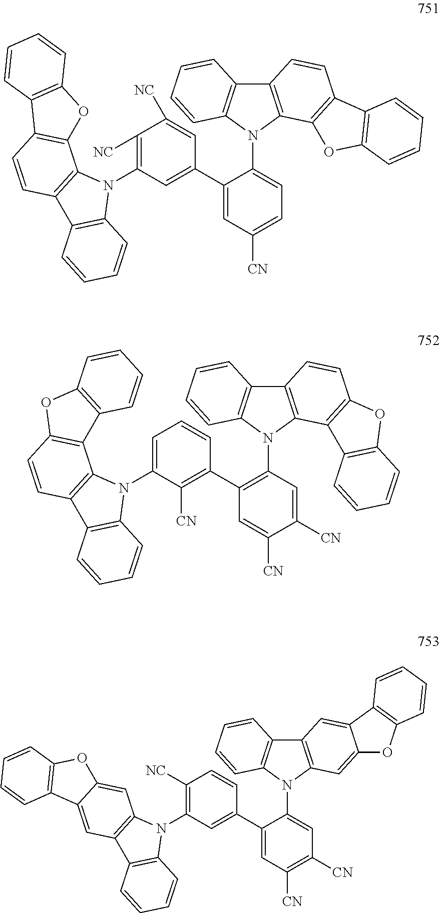 [SMILES: N#Cc1ccc(-n2c3ccccc3c3ccc4c5ccccc5oc4c32)c(-c2cc(C#N)c(C#N)c(-n3c4ccccc4c4ccc5c6ccccc6oc5c43)c2)c1 ; N#Cc1cc(-c2cccc(-n3c4ccccc4c4ccc5oc6ccccc6c5c43)c2C#N)c(-n2c3ccccc3c3ccc4oc5ccccc5c4c32)cc1C#N ; N#Cc1cc(-c2ccc(C#N)c(-n3c4ccccc4c4cc5c(cc43)oc3ccccc35)c2)c(-n2c3ccccc3c3cc4c(cc32)oc2ccccc24)cc1C#N]

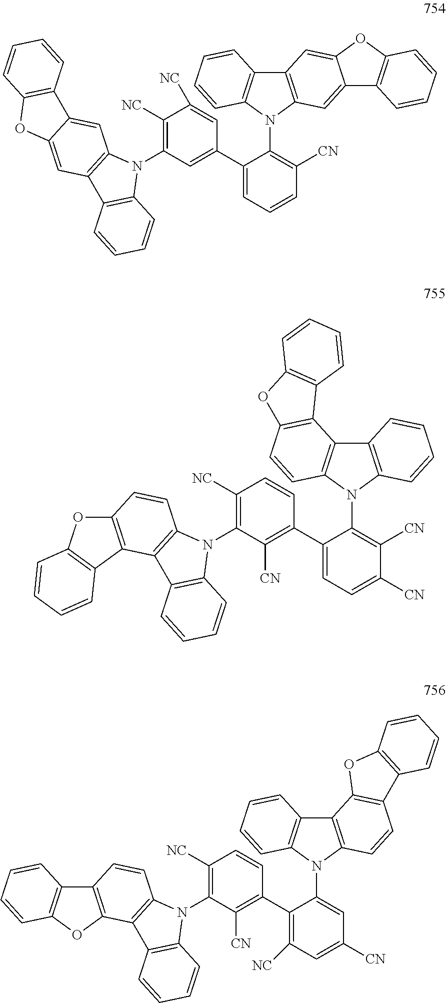 [SMILES: N#Cc1cc(-c2cccc(C#N)c2-n2c3ccccc3c3cc4oc5ccccc5c4cc32)cc(-n2c3ccccc3c3cc4oc5ccccc5c4cc32)c1C#N ; N#Cc1ccc(-c2ccc(C#N)c(-n3c4ccccc4c4c5c(ccc43)oc3ccccc35)c2C#N)c(-n2c3ccccc3c3c4c(ccc32)oc2ccccc24)c1C#N ; N#Cc1cc(C#N)c(-c2ccc(C#N)c(-n3c4ccccc4c4c5oc6ccccc6c5ccc43)c2C#N)c(-n2c3ccccc3c3c4oc5ccccc5c4ccc32)c1]

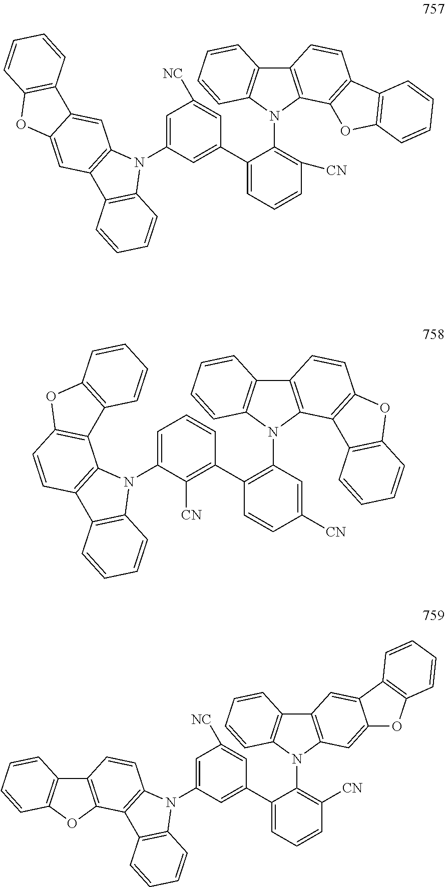 [SMILES: N#Cc1cc(-c2cccc(C#N)c2-n2c3ccccc3c3ccc4c5ccccc5oc4c32)cc(-n2c3ccccc3c3cc4oc5ccccc5c4cc32)c1 ; N#Cc1ccc(-c2cccc(-n3c4ccccc4c4ccc5oc6ccccc6c5c43)c2C#N)c(-n2c3ccccc3c3ccc4oc5ccccc5c4c32)c1 ; N#Cc1cc(-c2cccc(C#N)c2-n2c3ccccc3c3cc4c(cc32)oc2ccccc24)cc(-n2c3ccccc3c3c4oc5ccccc5c4ccc32)c1]

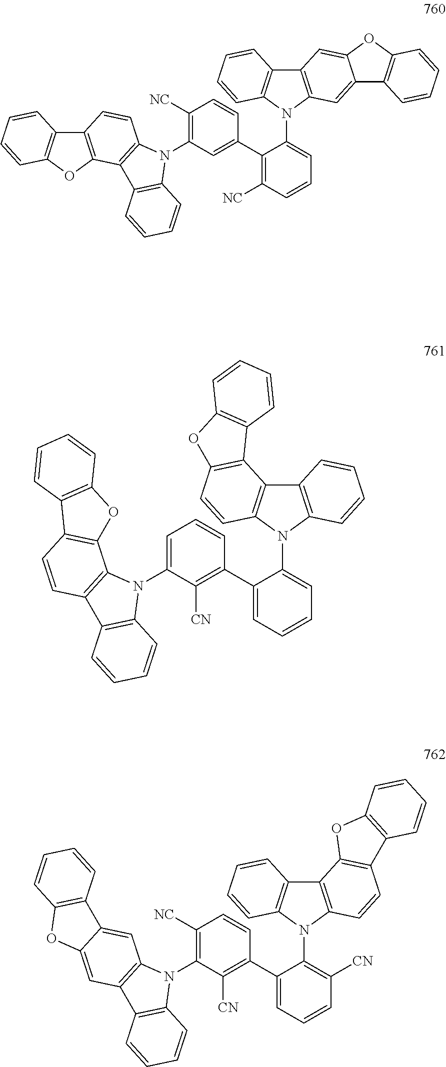 [SMILES: N#Cc1ccc(-c2c(C#N)cccc2-n2c3ccccc3c3cc4oc5ccccc5c4cc32)cc1-n1c2ccccc2c2c3oc4ccccc4c3ccc21 ; N#Cc1c(-c2ccccc2-n2c3ccccc3c3c4c(ccc32)oc2ccccc24)cccc1-n1c2ccccc2c2ccc3c4ccccc4oc3c21 ; N#Cc1ccc(-c2cccc(C#N)c2-n2c3ccccc3c3c4oc5ccccc5c4ccc32)c(C#N)c1-n1c2ccccc2c2cc3oc4ccccc4c3cc21]

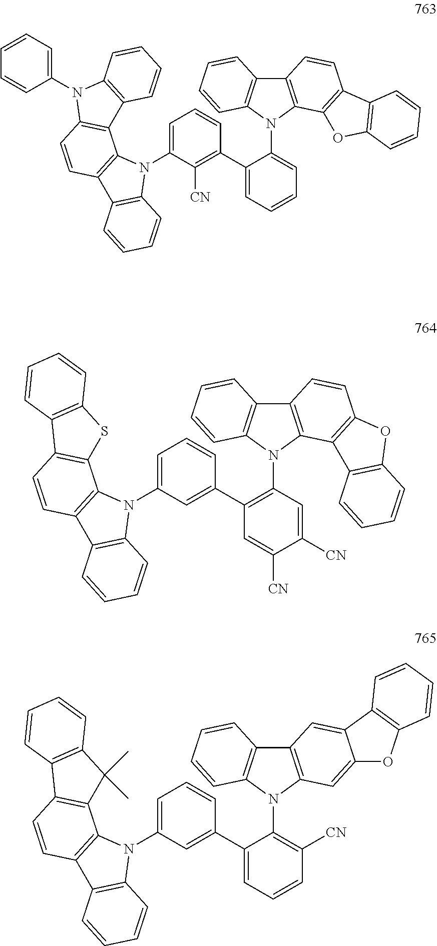 [SMILES: N#Cc1c(-c2ccccc2-n2c3ccccc3c3ccc4c5ccccc5oc4c32)cccc1-n1c2ccccc2c2ccc3c(c4ccccc4n3-c3ccccc3)c21 ; N#Cc1cc(-c2cccc(-n3c4ccccc4c4ccc5c6ccccc6sc5c43)c2)c(-n2c3ccccc3c3ccc4oc5ccccc5c4c32)cc1C#N ; CC1(C)c2ccccc2-c2ccc3c4ccccc4n(-c4cccc(-c5cccc(C#N)c5-n5c6ccccc6c6cc7c(cc65)oc5ccccc57)c4)c3c21]

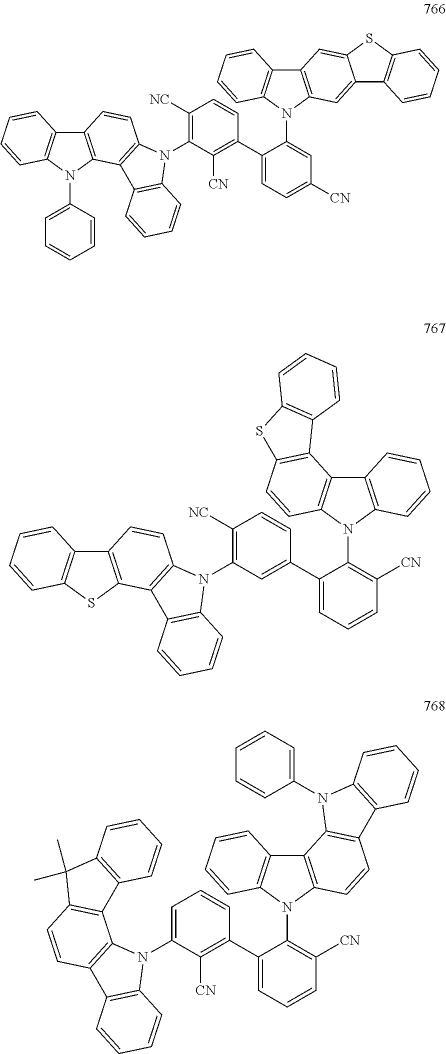 [SMILES: N#Cc1ccc(-c2ccc(C#N)c(-n3c4ccccc4c4c3ccc3c5ccccc5n(-c5ccccc5)c34)c2C#N)c(-n2c3ccccc3c3cc4sc5ccccc5c4cc32)c1 ; N#Cc1ccc(-c2cccc(C#N)c2-n2c3ccccc3c3c4c(ccc32)sc2ccccc24)cc1-n1c2ccccc2c2c3sc4ccccc4c3ccc21 ; CC1(C)c2ccccc2-c2c1ccc1c3ccccc3n(-c3cccc(-c4cccc(C#N)c4-n4c5ccccc5c5c4ccc4c6ccccc6n(-c6ccccc6)c45)c3C#N)c21]

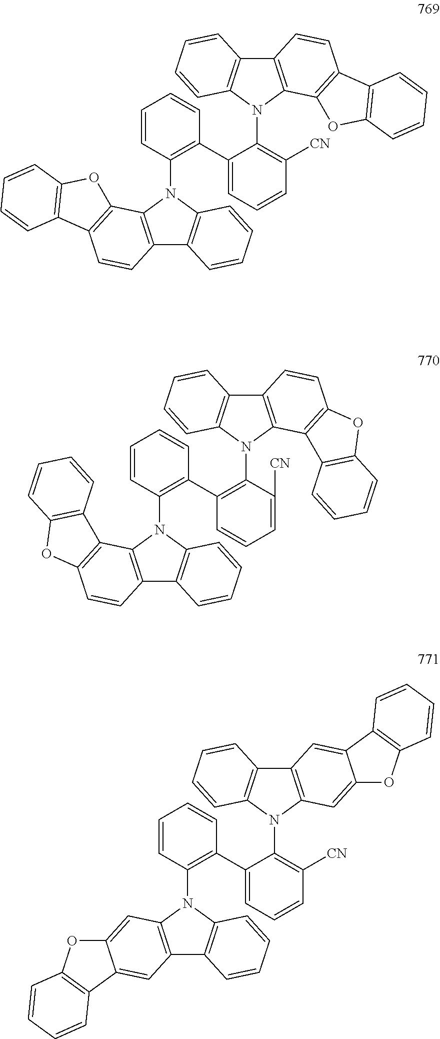 [SMILES: N#Cc1cccc(-c2ccccc2-n2c3ccccc3c3ccc4c5ccccc5oc4c32)c1-n1c2ccccc2c2ccc3c4ccccc4oc3c21 ; N#Cc1cccc(-c2ccccc2-n2c3ccccc3c3ccc4oc5ccccc5c4c32)c1-n1c2ccccc2c2ccc3oc4ccccc4c3c21 ; N#Cc1cccc(-c2ccccc2-n2c3ccccc3c3cc4c(cc32)oc2ccccc24)c1-n1c2ccccc2c2cc3c(cc21)oc1ccccc13]

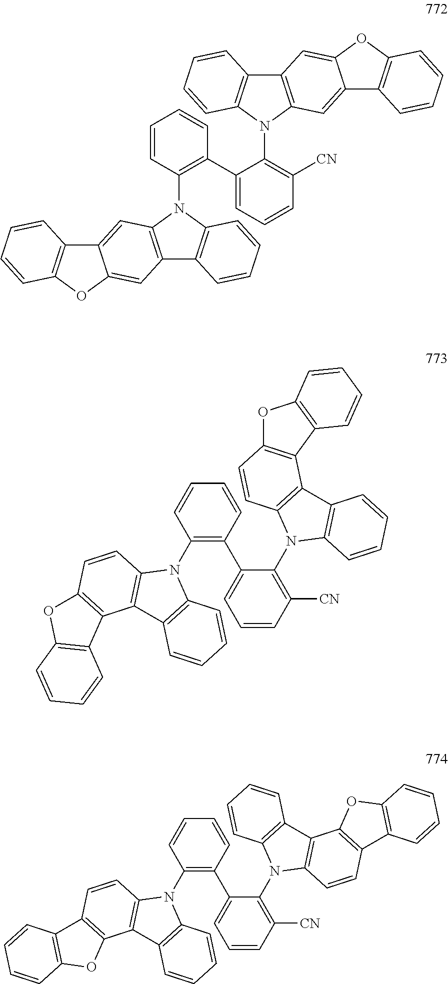 [SMILES: N#Cc1cccc(-c2ccccc2-n2c3ccccc3c3cc4oc5ccccc5c4cc32)c1-n1c2ccccc2c2cc3oc4ccccc4c3cc21 ; N#Cc1cccc(-c2ccccc2-n2c3ccccc3c3c4c(ccc32)oc2ccccc24)c1-n1c2ccccc2c2c3c(ccc21)oc1ccccc13 ; N#Cc1cccc(-c2ccccc2-n2c3ccccc3c3c4oc5ccccc5c4ccc32)c1-n1c2ccccc2c2c3oc4ccccc4c3ccc21]

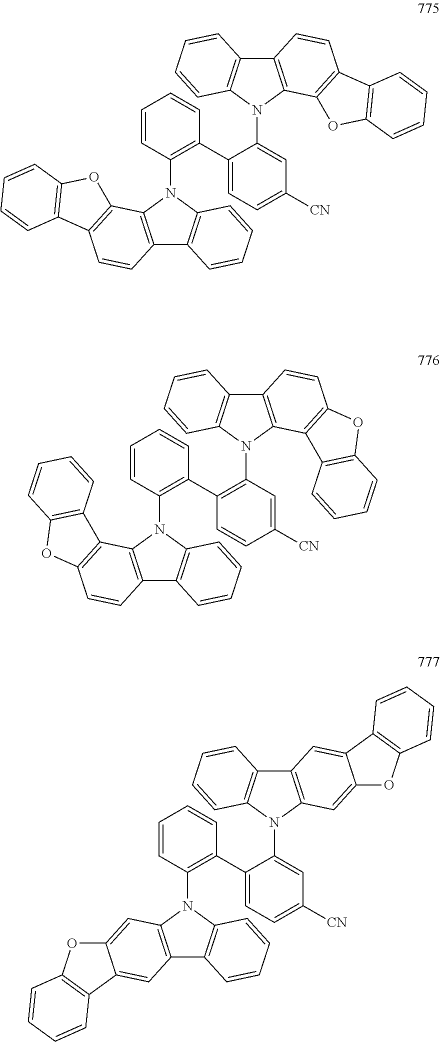 [SMILES: N#Cc1ccc(-c2ccccc2-n2c3ccccc3c3ccc4c5ccccc5oc4c32)c(-n2c3ccccc3c3ccc4c5ccccc5oc4c32)c1 ; N#Cc1ccc(-c2ccccc2-n2c3ccccc3c3ccc4oc5ccccc5c4c32)c(-n2c3ccccc3c3ccc4oc5ccccc5c4c32)c1 ; N#Cc1ccc(-c2ccccc2-n2c3ccccc3c3cc4c(cc32)oc2ccccc24)c(-n2c3ccccc3c3cc4c(cc32)oc2ccccc24)c1]

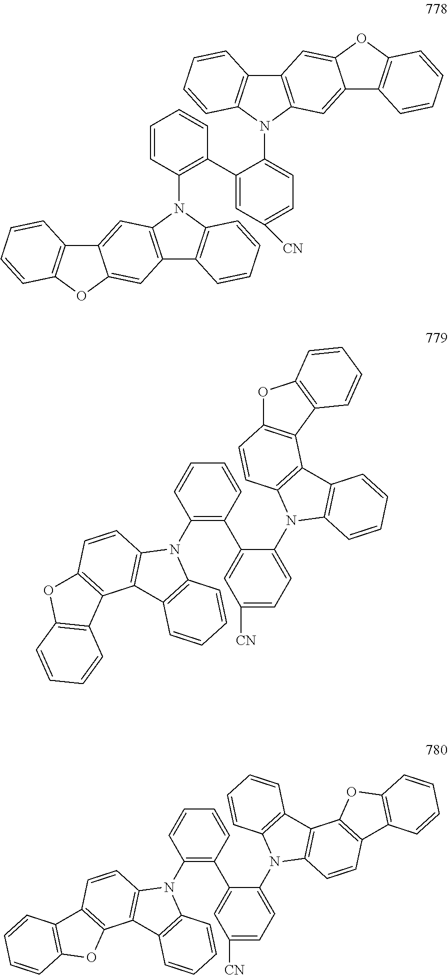 [SMILES: N#Cc1ccc(-n2c3ccccc3c3cc4oc5ccccc5c4cc32)c(-c2ccccc2-n2c3ccccc3c3cc4oc5ccccc5c4cc32)c1 ; N#Cc1ccc(-n2c3ccccc3c3c4c(ccc32)oc2ccccc24)c(-c2ccccc2-n2c3ccccc3c3c4c(ccc32)oc2ccccc24)c1 ; N#Cc1ccc(-n2c3ccccc3c3c4oc5ccccc5c4ccc32)c(-c2ccccc2-n2c3ccccc3c3c4oc5ccccc5c4ccc32)c1]

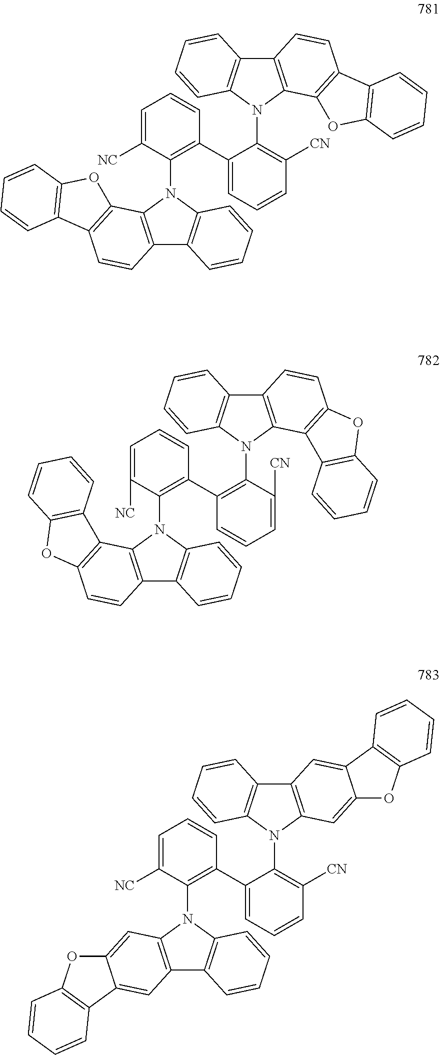 [SMILES: N#Cc1cccc(-c2cccc(C#N)c2-n2c3ccccc3c3ccc4c5ccccc5oc4c32)c1-n1c2ccccc2c2ccc3c4ccccc4oc3c21 ; N#Cc1cccc(-c2cccc(C#N)c2-n2c3ccccc3c3ccc4oc5ccccc5c4c32)c1-n1c2ccccc2c2ccc3oc4ccccc4c3c21 ; N#Cc1cccc(-c2cccc(C#N)c2-n2c3ccccc3c3cc4c(cc32)oc2ccccc24)c1-n1c2ccccc2c2cc3c(cc21)oc1ccccc13]

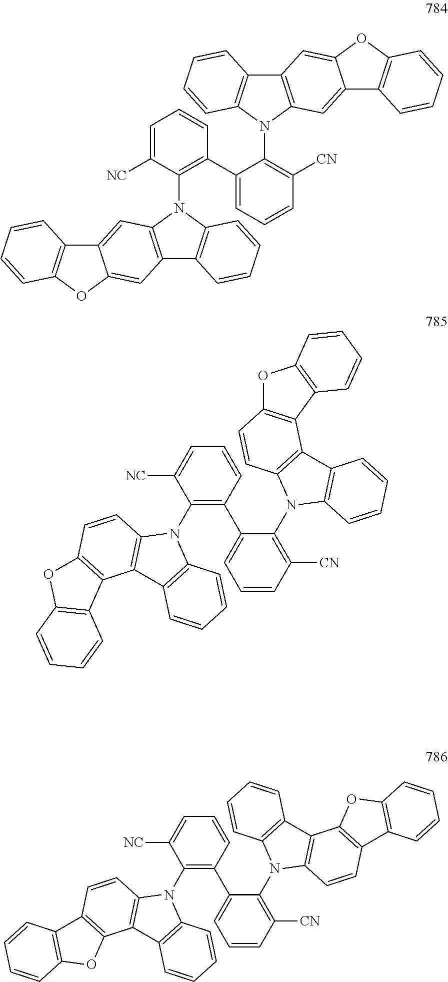 [SMILES: N#Cc1cccc(-c2cccc(C#N)c2-n2c3ccccc3c3cc4oc5ccccc5c4cc32)c1-n1c2ccccc2c2cc3oc4ccccc4c3cc21 ; N#Cc1cccc(-c2cccc(C#N)c2-n2c3ccccc3c3c4c(ccc32)oc2ccccc24)c1-n1c2ccccc2c2c3c(ccc21)oc1ccccc13 ; N#Cc1cccc(-c2cccc(C#N)c2-n2c3ccccc3c3c4oc5ccccc5c4ccc32)c1-n1c2ccccc2c2c3oc4ccccc4c3ccc21]

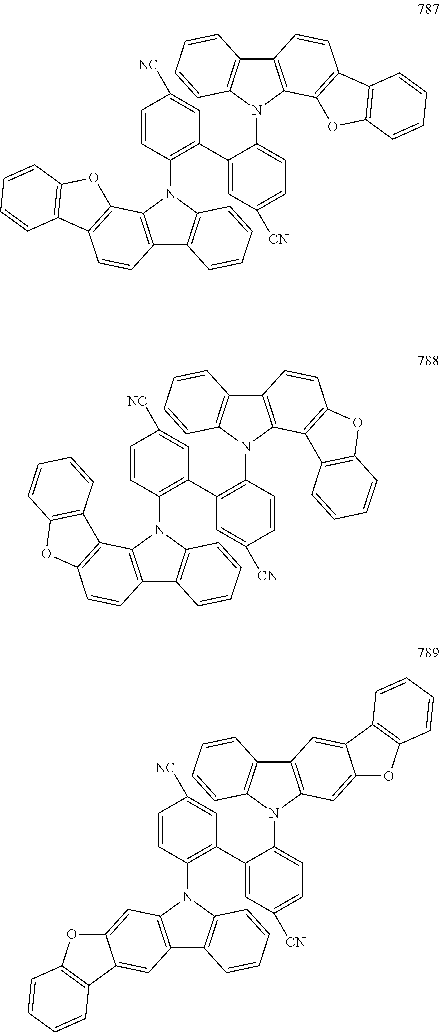 [SMILES: N#Cc1ccc(-n2c3ccccc3c3ccc4c5ccccc5oc4c32)c(-c2cc(C#N)ccc2-n2c3ccccc3c3ccc4c5ccccc5oc4c32)c1 ; N#Cc1ccc(-n2c3ccccc3c3ccc4oc5ccccc5c4c32)c(-c2cc(C#N)ccc2-n2c3ccccc3c3ccc4oc5ccccc5c4c32)c1 ; N#Cc1ccc(-n2c3ccccc3c3cc4c(cc32)oc2ccccc24)c(-c2cc(C#N)ccc2-n2c3ccccc3c3cc4c(cc32)oc2ccccc24)c1]

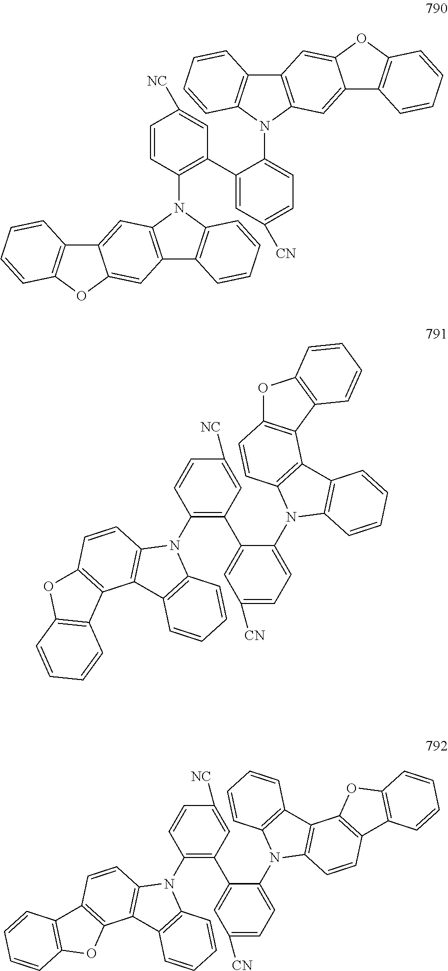 [SMILES: N#Cc1ccc(-n2c3ccccc3c3cc4oc5ccccc5c4cc32)c(-c2cc(C#N)ccc2-n2c3ccccc3c3cc4oc5ccccc5c4cc32)c1 ; N#Cc1ccc(-n2c3ccccc3c3c4c(ccc32)oc2ccccc24)c(-c2cc(C#N)ccc2-n2c3ccccc3c3c4c(ccc32)oc2ccccc24)c1 ; N#Cc1ccc(-n2c3ccccc3c3c4oc5ccccc5c4ccc32)c(-c2cc(C#N)ccc2-n2c3ccccc3c3c4oc5ccccc5c4ccc32)c1]

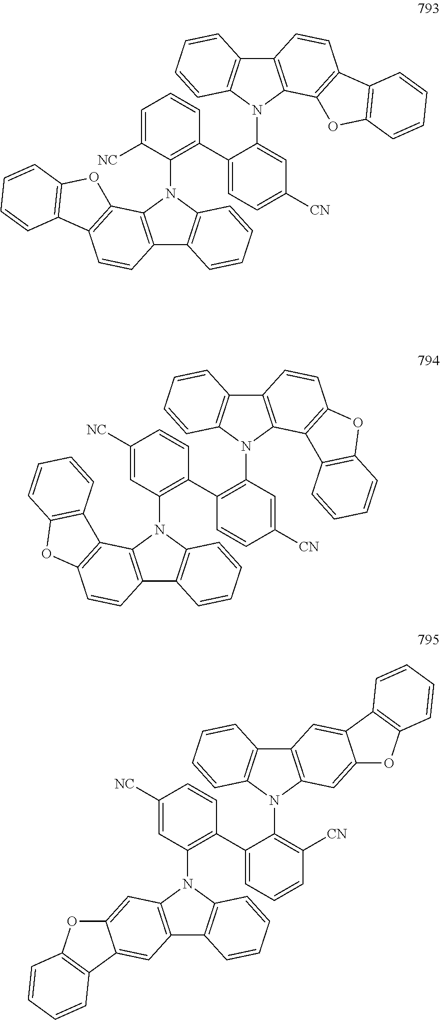 [SMILES: N#Cc1ccc(-c2cccc(C#N)c2-n2c3ccccc3c3ccc4c5ccccc5oc4c32)c(-n2c3ccccc3c3ccc4c5ccccc5oc4c32)c1 ; N#Cc1ccc(-c2ccc(C#N)cc2-n2c3ccccc3c3ccc4oc5ccccc5c4c32)c(-n2c3ccccc3c3ccc4oc5ccccc5c4c32)c1 ; N#Cc1ccc(-c2cccc(C#N)c2-n2c3ccccc3c3cc4c(cc32)oc2ccccc24)c(-n2c3ccccc3c3cc4c(cc32)oc2ccccc24)c1]

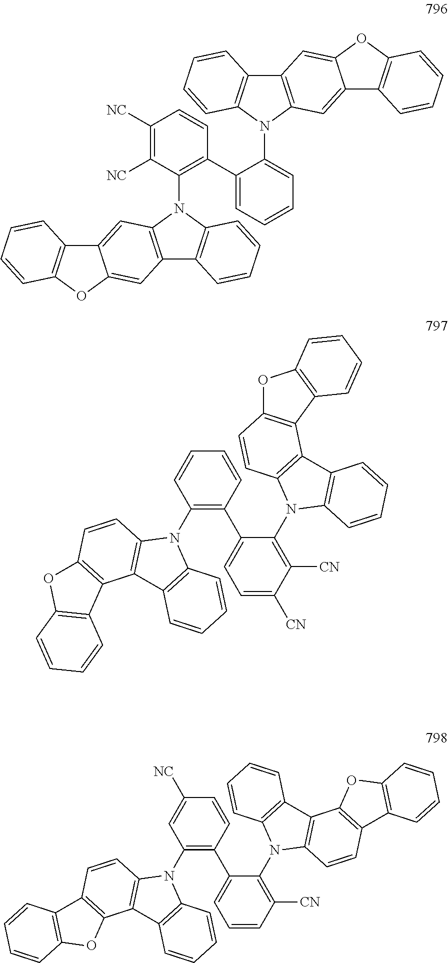 [SMILES: N#Cc1ccc(-c2ccccc2-n2c3ccccc3c3cc4oc5ccccc5c4cc32)c(-n2c3ccccc3c3cc4oc5ccccc5c4cc32)c1C#N ; N#Cc1ccc(-c2ccccc2-n2c3ccccc3c3c4c(ccc32)oc2ccccc24)c(-n2c3ccccc3c3c4c(ccc32)oc2ccccc24)c1C#N ; N#Cc1ccc(-c2cccc(C#N)c2-n2c3ccccc3c3c4oc5ccccc5c4ccc32)c(-n2c3ccccc3c3c4oc5ccccc5c4ccc32)c1]

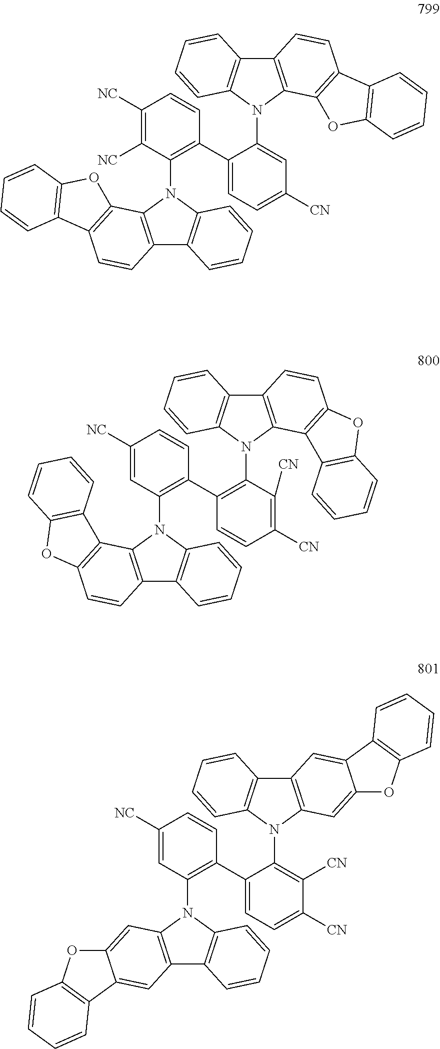 [SMILES: N#Cc1ccc(-c2ccc(C#N)c(C#N)c2-n2c3ccccc3c3ccc4c5ccccc5oc4c32)c(-n2c3ccccc3c3ccc4c5ccccc5oc4c32)c1 ; N#Cc1ccc(-c2ccc(C#N)c(C#N)c2-n2c3ccccc3c3ccc4oc5ccccc5c4c32)c(-n2c3ccccc3c3ccc4oc5ccccc5c4c32)c1 ; N#Cc1ccc(-c2ccc(C#N)c(C#N)c2-n2c3ccccc3c3cc4c(cc32)oc2ccccc24)c(-n2c3ccccc3c3cc4c(cc32)oc2ccccc24)c1]

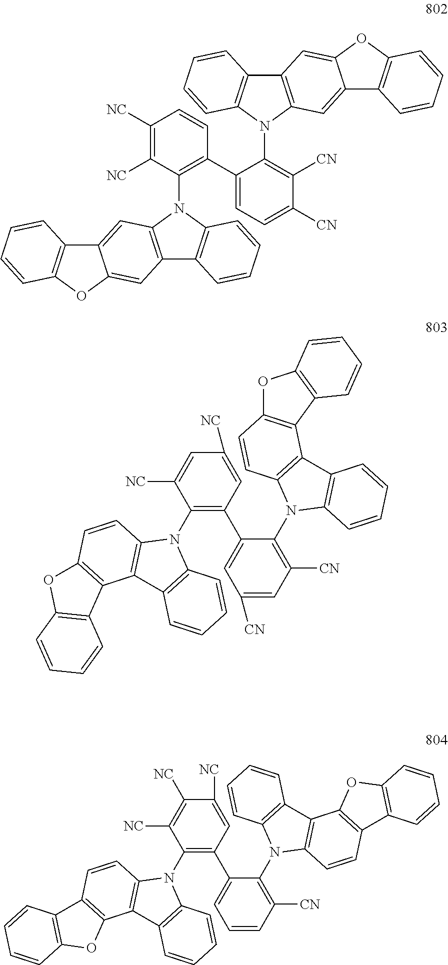 [SMILES: N#Cc1ccc(-c2ccc(C#N)c(C#N)c2-n2c3ccccc3c3cc4oc5ccccc5c4cc32)c(-n2c3ccccc3c3cc4oc5ccccc5c4cc32)c1C#N ; N#Cc1cc(C#N)c(-n2c3ccccc3c3c4c(ccc32)oc2ccccc24)c(-c2cc(C#N)cc(C#N)c2-n2c3ccccc3c3c4c(ccc32)oc2ccccc24)c1 ; N#Cc1cc(-c2cccc(C#N)c2-n2c3ccccc3c3c4oc5ccccc5c4ccc32)c(-n2c3ccccc3c3c4oc5ccccc5c4ccc32)c(C#N)c1C#N]

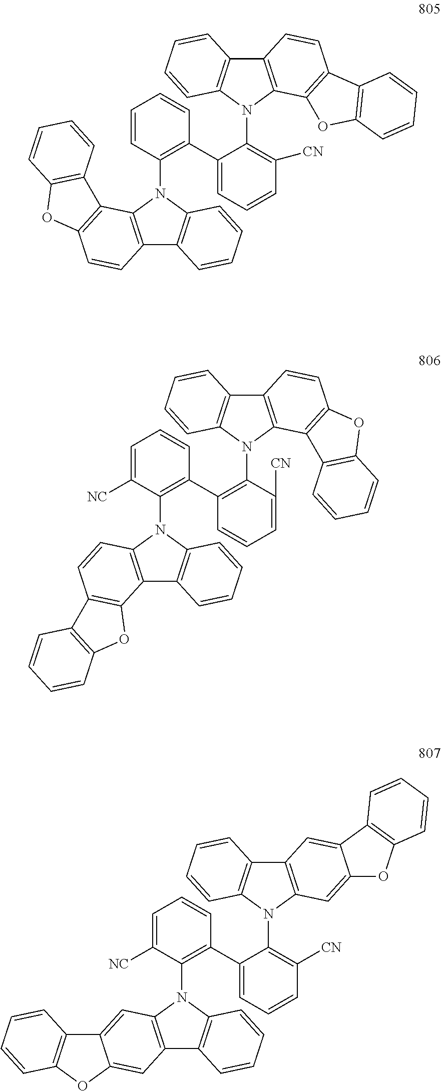 [SMILES: N#Cc1cccc(-c2ccccc2-n2c3ccccc3c3ccc4oc5ccccc5c4c32)c1-n1c2ccccc2c2ccc3c4ccccc4oc3c21 ; N#Cc1cccc(-c2cccc(C#N)c2-n2c3ccccc3c3ccc4oc5ccccc5c4c32)c1-n1c2ccccc2c2c3oc4ccccc4c3ccc21 ; N#Cc1cccc(-c2cccc(C#N)c2-n2c3ccccc3c3cc4oc5ccccc5c4cc32)c1-n1c2ccccc2c2cc3c(cc21)oc1ccccc13]

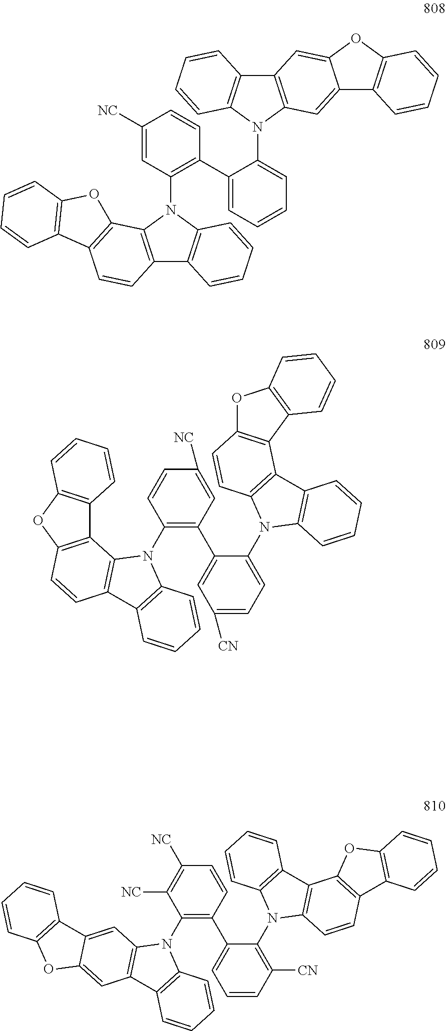 [SMILES: N#Cc1ccc(-c2ccccc2-n2c3ccccc3c3cc4oc5ccccc5c4cc32)c(-n2c3ccccc3c3ccc4c5ccccc5oc4c32)c1 ; N#Cc1ccc(-n2c3ccccc3c3c4c(ccc32)oc2ccccc24)c(-c2cc(C#N)ccc2-n2c3ccccc3c3ccc4oc5ccccc5c4c32)c1 ; N#Cc1ccc(-c2cccc(C#N)c2-n2c3ccccc3c3c4oc5ccccc5c4ccc32)c(-n2c3ccccc3c3cc4oc5ccccc5c4cc32)c1C#N]

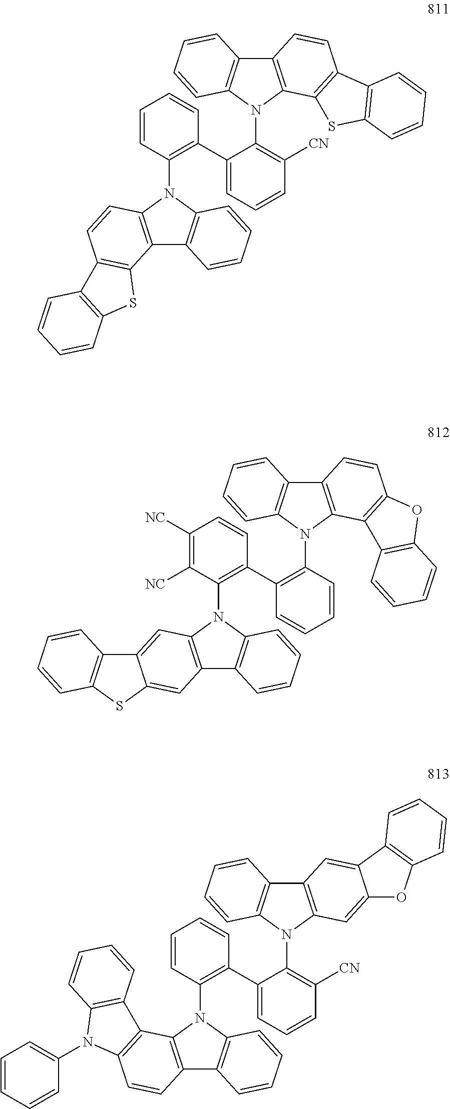 [SMILES: N#Cc1cccc(-c2ccccc2-n2c3ccccc3c3c4sc5ccccc5c4ccc32)c1-n1c2ccccc2c2ccc3c4ccccc4sc3c21 ; N#Cc1ccc(-c2ccccc2-n2c3ccccc3c3ccc4oc5ccccc5c4c32)c(-n2c3ccccc3c3cc4sc5ccccc5c4cc32)c1C#N ; N#Cc1cccc(-c2ccccc2-n2c3ccccc3c3ccc4c(c5ccccc5n4-c4ccccc4)c32)c1-n1c2ccccc2c2cc3c(cc21)oc1ccccc13]

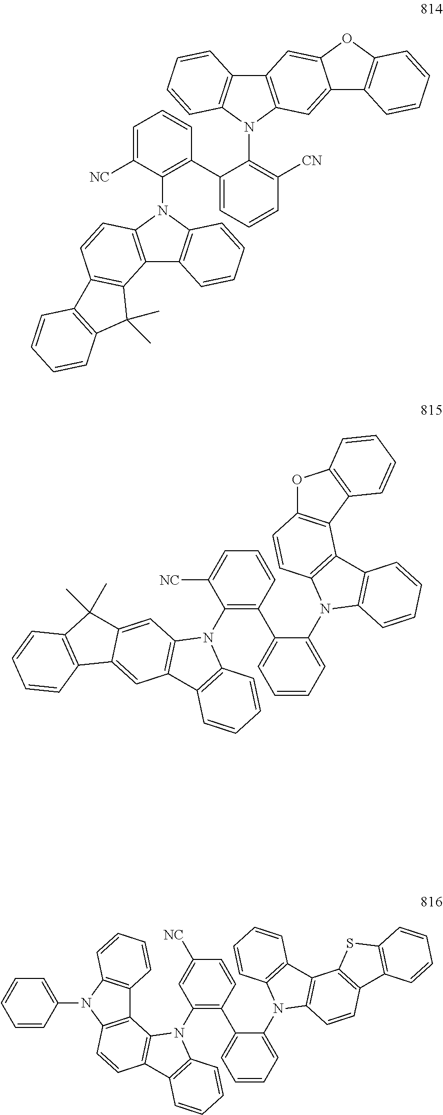 [SMILES: CC1(C)c2ccccc2-c2ccc3c(c21)c1ccccc1n3-c1c(C#N)cccc1-c1cccc(C#N)c1-n1c2ccccc2c2cc3oc4ccccc4c3cc21 ; CC1(C)c2ccccc2-c2cc3c4ccccc4n(-c4c(C#N)cccc4-c4ccccc4-n4c5ccccc5c5c6c(ccc54)oc4ccccc46)c3cc21 ; N#Cc1ccc(-c2ccccc2-n2c3ccccc3c3c4sc5ccccc5c4ccc32)c(-n2c3ccccc3c3ccc4c(c5ccccc5n4-c4ccccc4)c32)c1]

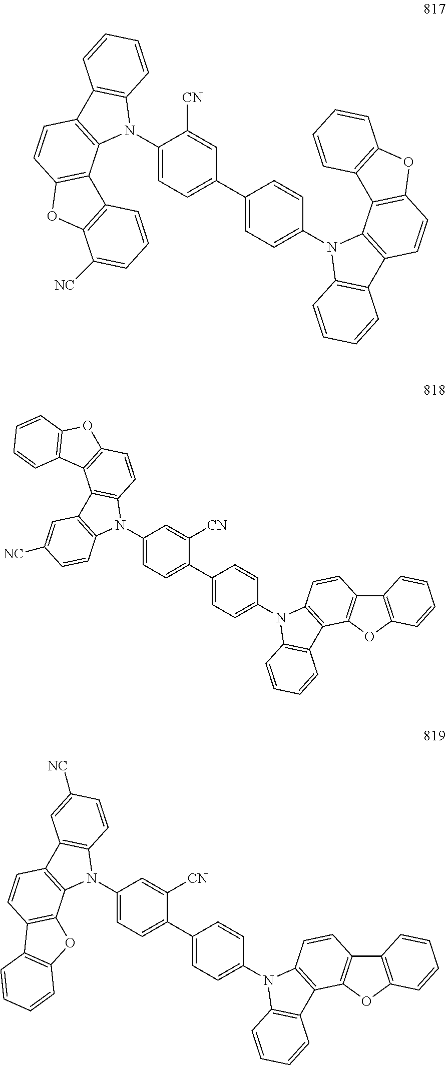 [SMILES: N#Cc1cc(-c2ccc(-n3c4ccccc4c4ccc5oc6ccccc6c5c43)cc2)ccc1-n1c2ccccc2c2ccc3oc4c(C#N)cccc4c3c21 ; N#Cc1ccc2c(c1)c1c3c(ccc1n2-c1ccc(-c2ccc(-n4c5ccccc5c5c6oc7ccccc7c6ccc54)cc2)c(C#N)c1)oc1ccccc13 ; N#Cc1ccc2c(c1)c1ccc3c4ccccc4oc3c1n2-c1ccc(-c2ccc(-n3c4ccccc4c4c5oc6ccccc6c5ccc43)cc2)c(C#N)c1]

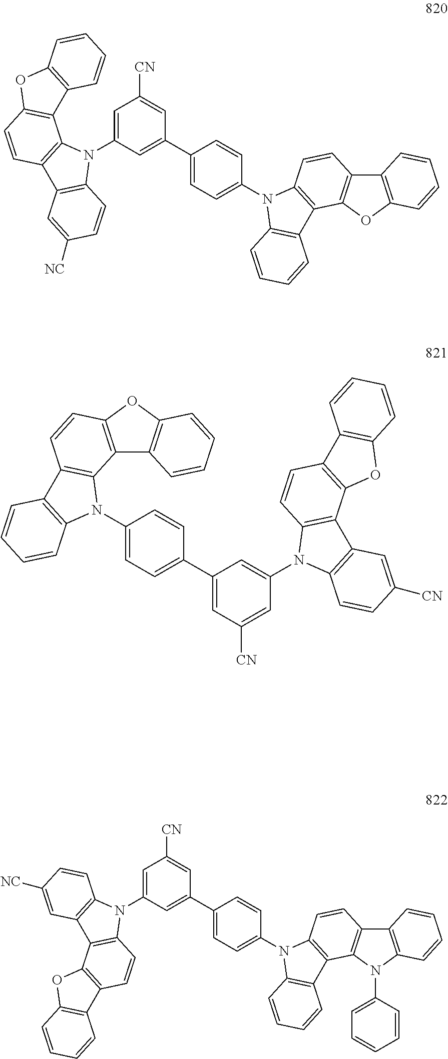 [SMILES: N#Cc1cc(-c2ccc(-n3c4ccccc4c4c5oc6ccccc6c5ccc43)cc2)cc(-n2c3ccc(C#N)cc3c3ccc4oc5ccccc5c4c32)c1 ; N#Cc1cc(-c2ccc(-n3c4ccccc4c4ccc5oc6ccccc6c5c43)cc2)cc(-n2c3ccc(C#N)cc3c3c4oc5ccccc5c4ccc32)c1 ; N#Cc1cc(-c2ccc(-n3c4ccccc4c4c3ccc3c5ccccc5n(-c5ccccc5)c34)cc2)cc(-n2c3ccc(C#N)cc3c3c4oc5ccccc5c4ccc32)c1]

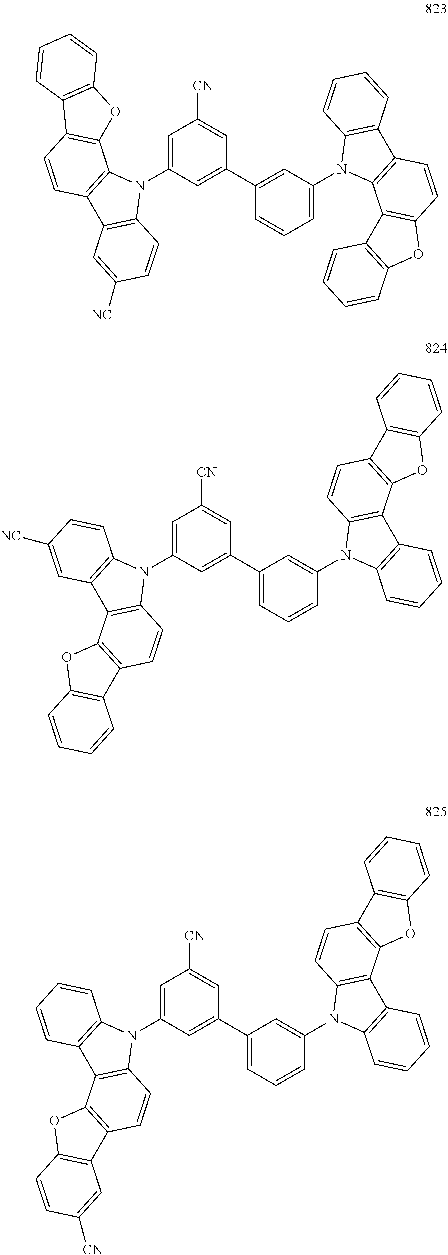 [SMILES: N#Cc1cc(-c2cccc(-n3c4ccccc4c4ccc5oc6ccccc6c5c43)c2)cc(-n2c3ccc(C#N)cc3c3ccc4c5ccccc5oc4c32)c1 ; N#Cc1cc(-c2cccc(-n3c4ccccc4c4c5oc6ccccc6c5ccc43)c2)cc(-n2c3ccc(C#N)cc3c3c4oc5ccccc5c4ccc32)c1 ; N#Cc1cc(-c2cccc(-n3c4ccccc4c4c5oc6ccccc6c5ccc43)c2)cc(-n2c3ccccc3c3c4oc5ccc(C#N)cc5c4ccc32)c1]

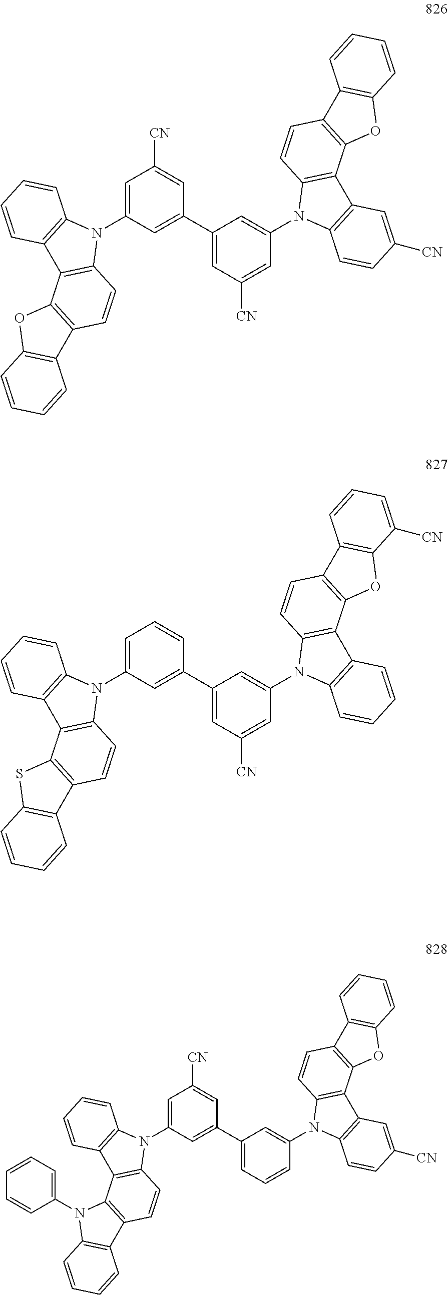 [SMILES: N#Cc1cc(-c2cc(C#N)cc(-n3c4ccc(C#N)cc4c4c5oc6ccccc6c5ccc43)c2)cc(-n2c3ccccc3c3c4oc5ccccc5c4ccc32)c1 ; N#Cc1cc(-c2cccc(-n3c4ccccc4c4c5sc6ccccc6c5ccc43)c2)cc(-n2c3ccccc3c3c4oc5c(C#N)cccc5c4ccc32)c1 ; N#Cc1cc(-c2cccc(-n3c4ccc(C#N)cc4c4c5oc6ccccc6c5ccc43)c2)cc(-n2c3ccccc3c3c2ccc2c4ccccc4n(-c4ccccc4)c23)c1]

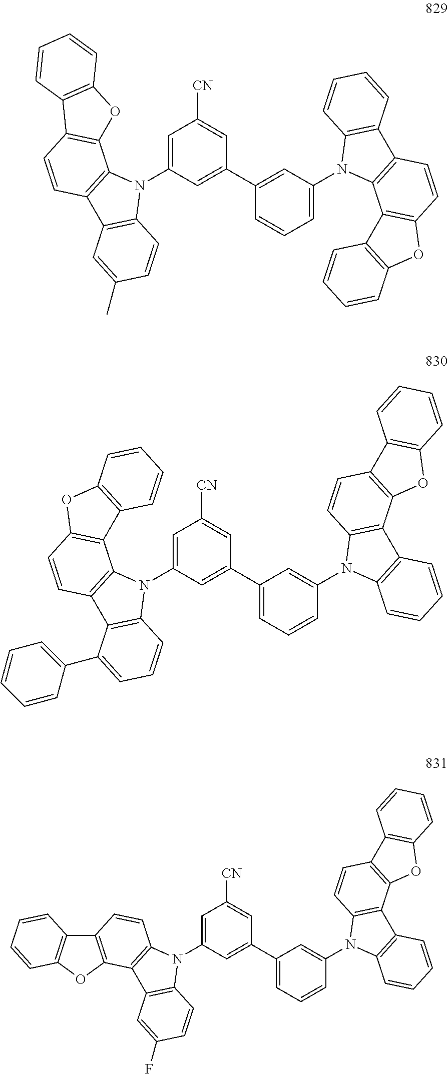 [SMILES: Cc1ccc2c(c1)c1ccc3c4ccccc4oc3c1n2-c1cc(C#N)cc(-c2cccc(-n3c4ccccc4c4ccc5oc6ccccc6c5c43)c2)c1 ; N#Cc1cc(-c2cccc(-n3c4ccccc4c4c5oc6ccccc6c5ccc43)c2)cc(-n2c3cccc(-c4ccccc4)c3c3ccc4oc5ccccc5c4c32)c1 ; N#Cc1cc(-c2cccc(-n3c4ccccc4c4c5oc6ccccc6c5ccc43)c2)cc(-n2c3ccc(F)cc3c3c4oc5ccccc5c4ccc32)c1]

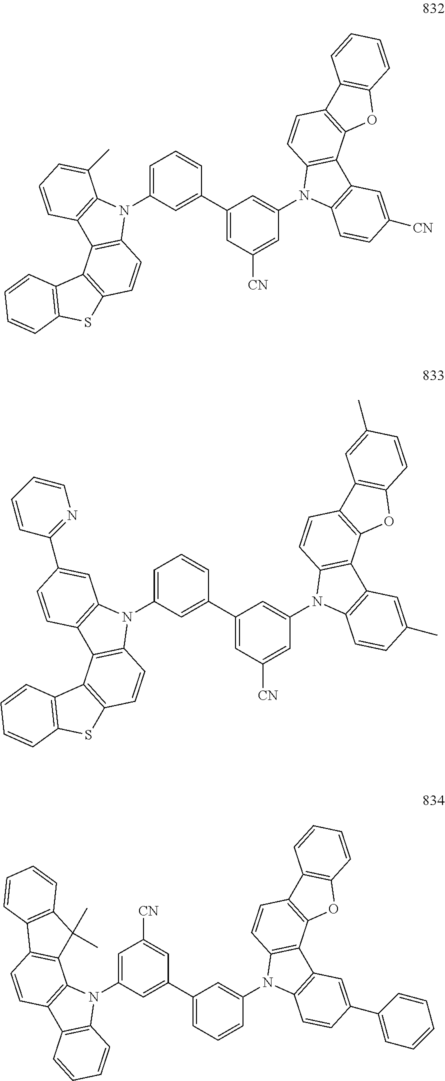 [SMILES: Cc1cccc2c3c4c(ccc3n(-c3cccc(-c5cc(C#N)cc(-n6c7ccc(C#N)cc7c7c8oc9ccccc9c8ccc76)c5)c3)c12)sc1ccccc14 ; Cc1ccc2oc3c(ccc4c3c3cc(C)ccc3n4-c3cc(C#N)cc(-c4cccc(-n5c6cc(-c7ccccn7)ccc6c6c7c(ccc65)sc5ccccc57)c4)c3)c2c1 ; CC1(C)c2ccccc2-c2ccc3c4ccccc4n(-c4cc(C#N)cc(-c5cccc(-n6c7ccc(-c8ccccc8)cc7c7c8oc9ccccc9c8ccc76)c5)c4)c3c21]

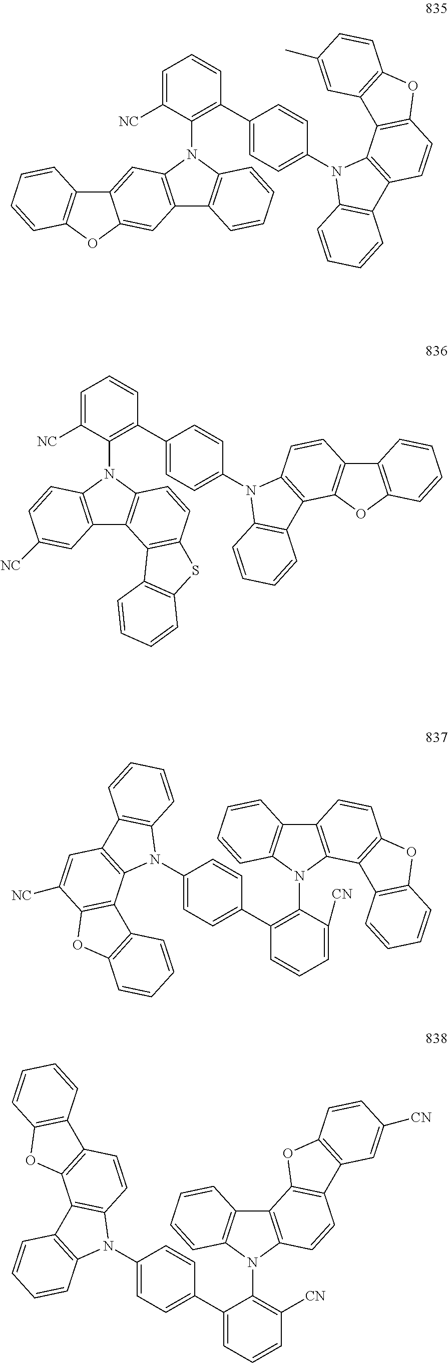 [SMILES: Cc1ccc2oc3ccc4c5ccccc5n(-c5ccc(-c6cccc(C#N)c6-n6c7ccccc7c7cc8oc9ccccc9c8cc76)cc5)c4c3c2c1 ; N#Cc1ccc2c(c1)c1c3c(ccc1n2-c1c(C#N)cccc1-c1ccc(-n2c4ccccc4c4c5oc6ccccc6c5ccc42)cc1)sc1ccccc13 ; N#Cc1cccc(-c2ccc(-n3c4ccccc4c4cc(C#N)c5oc6ccccc6c5c43)cc2)c1-n1c2ccccc2c2ccc3oc4ccccc4c3c21 ; N#Cc1ccc2oc3c(ccc4c3c3ccccc3n4-c3c(C#N)cccc3-c3ccc(-n4c5ccccc5c5c6oc7ccccc7c6ccc54)cc3)c2c1]

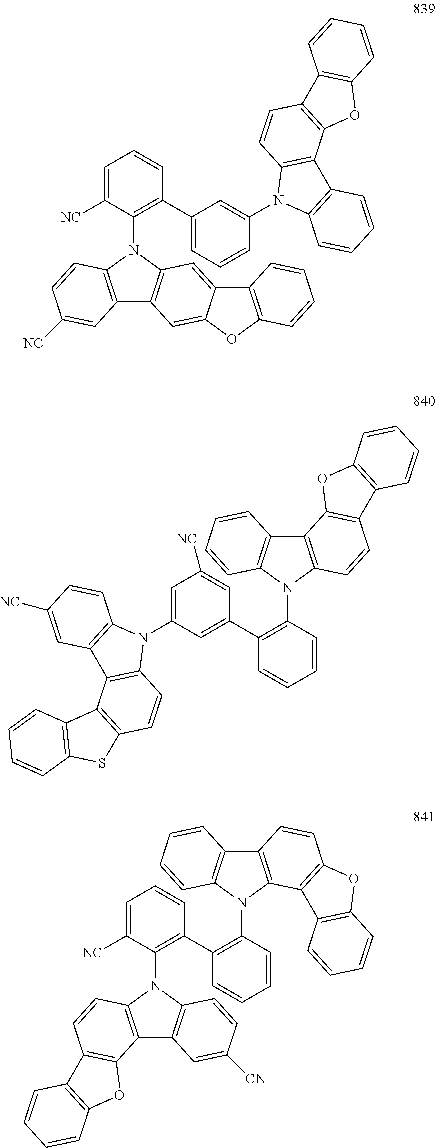 [SMILES: N#Cc1ccc2c(c1)c1cc3oc4ccccc4c3cc1n2-c1c(C#N)cccc1-c1cccc(-n2c3ccccc3c3c4oc5ccccc5c4ccc32)c1 ; N#Cc1cc(-c2ccccc2-n2c3ccccc3c3c4oc5ccccc5c4ccc32)cc(-n2c3ccc(C#N)cc3c3c4c(ccc32)sc2ccccc24)c1 ; N#Cc1ccc2c(c1)c1c3oc4ccccc4c3ccc1n2-c1c(C#N)cccc1-c1ccccc1-n1c2ccccc2c2ccc3oc4ccccc4c3c21]

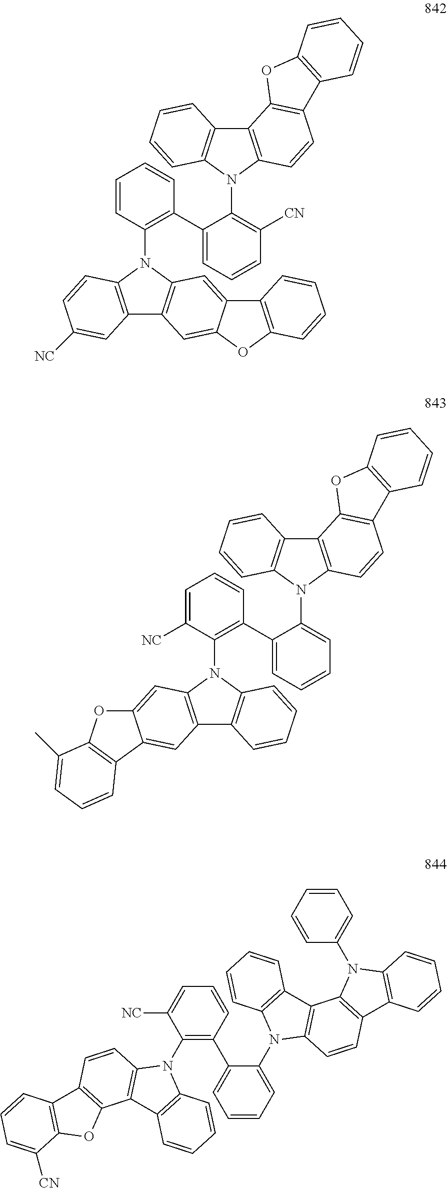 [SMILES: N#Cc1ccc2c(c1)c1cc3oc4ccccc4c3cc1n2-c1ccccc1-c1cccc(C#N)c1-n1c2ccccc2c2c3oc4ccccc4c3ccc21 ; Cc1cccc2c1oc1cc3c(cc12)c1ccccc1n3-c1c(C#N)cccc1-c1ccccc1-n1c2ccccc2c2c3oc4ccccc4c3ccc21 ; N#Cc1cccc(-c2ccccc2-n2c3ccccc3c3c2ccc2c4ccccc4n(-c4ccccc4)c23)c1-n1c2ccccc2c2c3oc4c(C#N)cccc4c3ccc21]

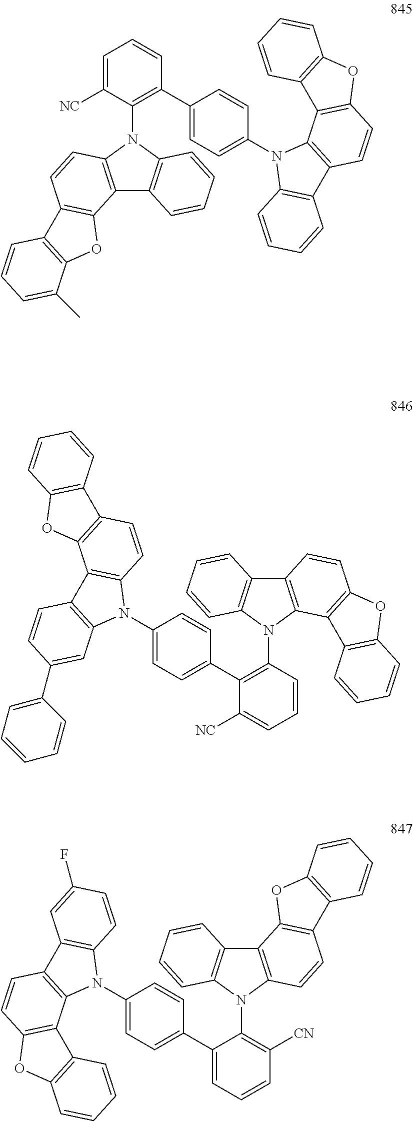 [SMILES: Cc1cccc2c1oc1c2ccc2c1c1ccccc1n2-c1c(C#N)cccc1-c1ccc(-n2c3ccccc3c3ccc4oc5ccccc5c4c32)cc1 ; N#Cc1cccc(-n2c3ccccc3c3ccc4oc5ccccc5c4c32)c1-c1ccc(-n2c3cc(-c4ccccc4)ccc3c3c4oc5ccccc5c4ccc32)cc1 ; N#Cc1cccc(-c2ccc(-n3c4ccc(F)cc4c4ccc5oc6ccccc6c5c43)cc2)c1-n1c2ccccc2c2c3oc4ccccc4c3ccc21]

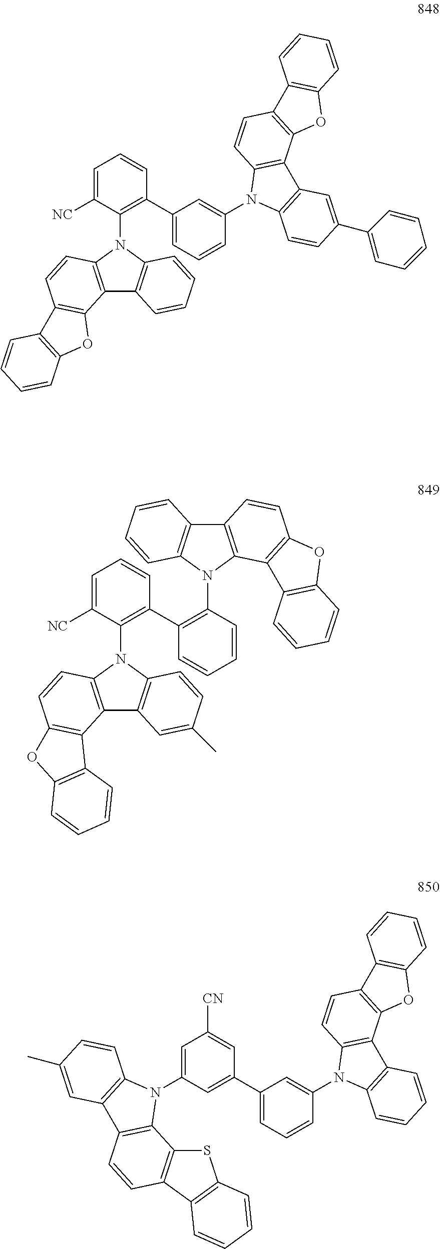 [SMILES: N#Cc1cccc(-c2cccc(-n3c4ccc(-c5ccccc5)cc4c4c5oc6ccccc6c5ccc43)c2)c1-n1c2ccccc2c2c3oc4ccccc4c3ccc21 ; Cc1ccc2c(c1)c1c3c(ccc1n2-c1c(C#N)cccc1-c1ccccc1-n1c2ccccc2c2ccc4oc5ccccc5c4c21)oc1ccccc13 ; Cc1ccc2c(c1)c1ccc3c4ccccc4sc3c1n2-c1cc(C#N)cc(-c2cccc(-n3c4ccccc4c4c5oc6ccccc6c5ccc43)c2)c1]

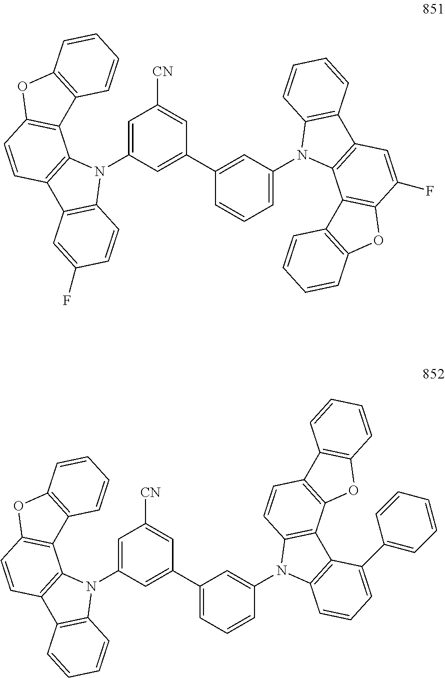 [SMILES: N#Cc1cc(-c2cccc(-n3c4ccccc4c4cc(F)c5oc6ccccc6c5c43)c2)cc(-n2c3ccc(F)cc3c3ccc4oc5ccccc5c4c32)c1 ; N#Cc1cc(-c2cccc(-n3c4cccc(-c5ccccc5)c4c4c5oc6ccccc6c5ccc43)c2)cc(-n2c3ccccc3c3ccc4oc5ccccc5c4c32)c1]

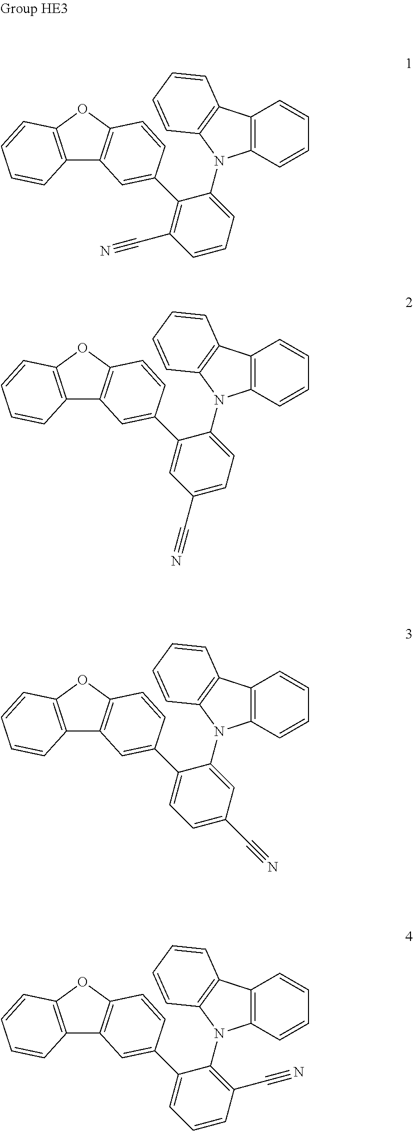 [SMILES: N#Cc1cccc(-n2c3ccccc3c3ccccc32)c1-c1ccc2oc3ccccc3c2c1 ; N#Cc1ccc(-n2c3ccccc3c3ccccc32)c(-c2ccc3oc4ccccc4c3c2)c1 ; N#Cc1ccc(-c2ccc3oc4ccccc4c3c2)c(-n2c3ccccc3c3ccccc32)c1 ; N#Cc1cccc(-c2ccc3oc4ccccc4c3c2)c1-n1c2ccccc2c2ccccc21]

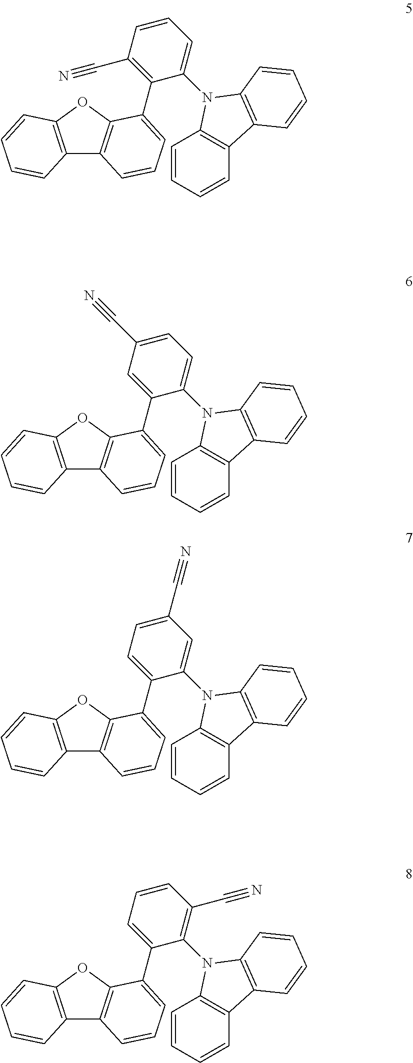

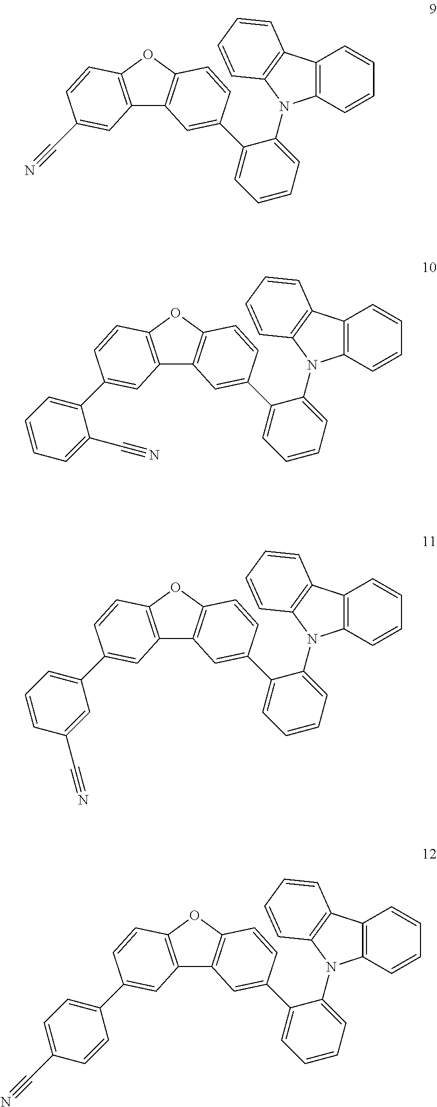 [SMILES: N#Cc1ccc2oc3ccc(-c4ccccc4-n4c5ccccc5c5ccccc54)cc3c2c1 ; N#Cc1ccccc1-c1ccc2oc3ccc(-c4ccccc4-n4c5ccccc5c5ccccc54)cc3c2c1 ; N#Cc1cccc(-c2ccc3oc4ccc(-c5ccccc5-n5c6ccccc6c6ccccc65)cc4c3c2)c1 ; N#Cc1ccc(-c2ccc3oc4ccc(-c5ccccc5-n5c6ccccc6c6ccccc65)cc4c3c2)cc1]

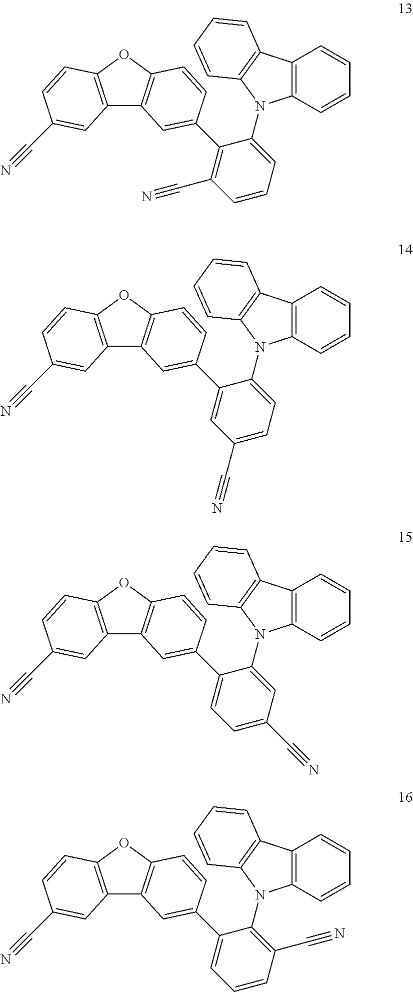

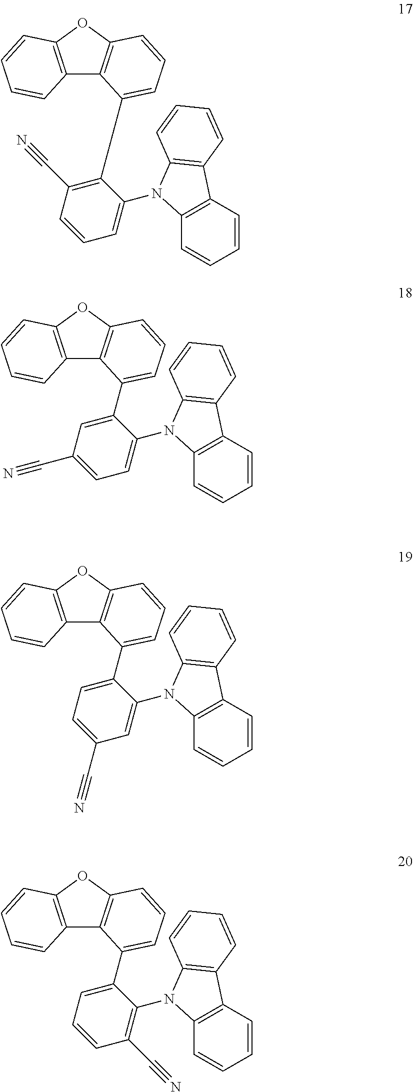 [SMILES: N#Cc1cccc(-n2c3ccccc3c3ccccc32)c1-c1cccc2oc3ccccc3c12 ; N#Cc1ccc(-n2c3ccccc3c3ccccc32)c(-c2cccc3oc4ccccc4c23)c1 ; N#Cc1ccc(-c2cccc3oc4ccccc4c23)c(-n2c3ccccc3c3ccccc32)c1 ; N#Cc1cccc(-c2cccc3oc4ccccc4c23)c1-n1c2ccccc2c2ccccc21]

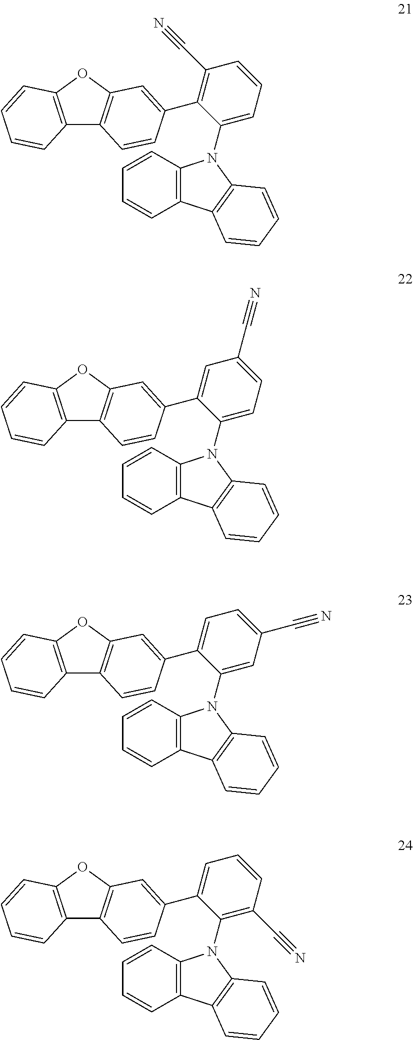 [SMILES: N#Cc1cccc(-n2c3ccccc3c3ccccc32)c1-c1ccc2c(c1)oc1ccccc12 ; N#Cc1ccc(-n2c3ccccc3c3ccccc32)c(-c2ccc3c(c2)oc2ccccc23)c1 ; N#Cc1ccc(-c2ccc3c(c2)oc2ccccc23)c(-n2c3ccccc3c3ccccc32)c1 ; N#Cc1cccc(-c2ccc3c(c2)oc2ccccc23)c1-n1c2ccccc2c2ccccc21]

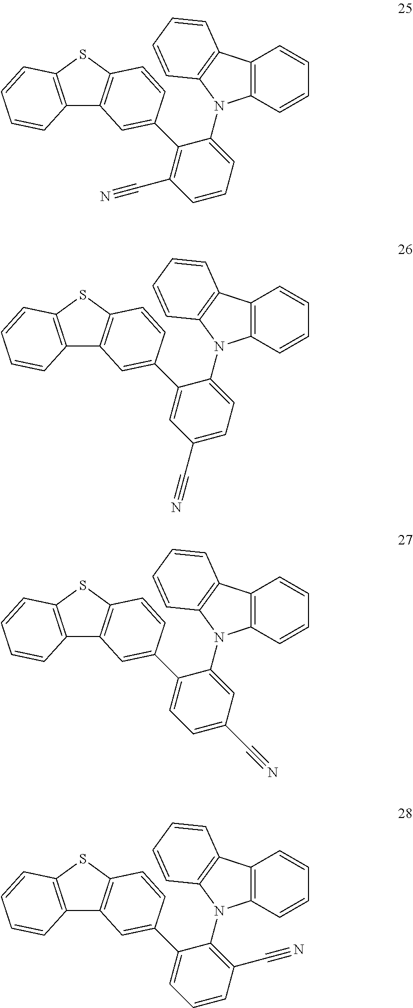

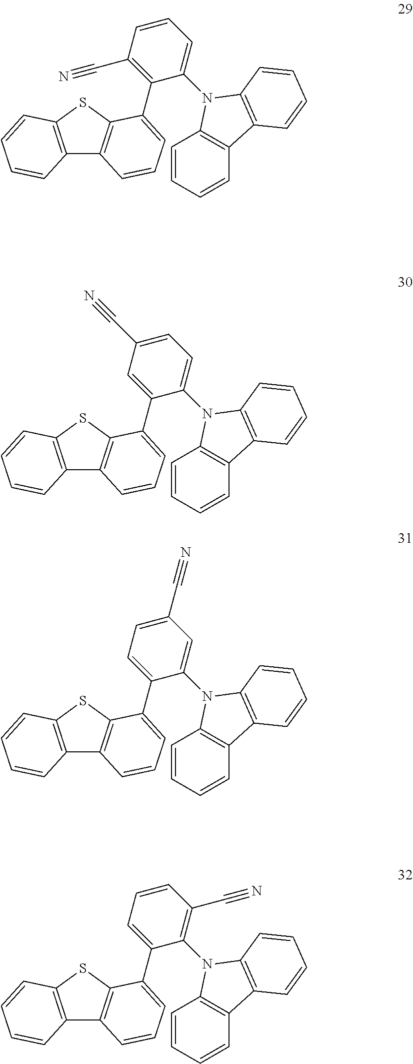

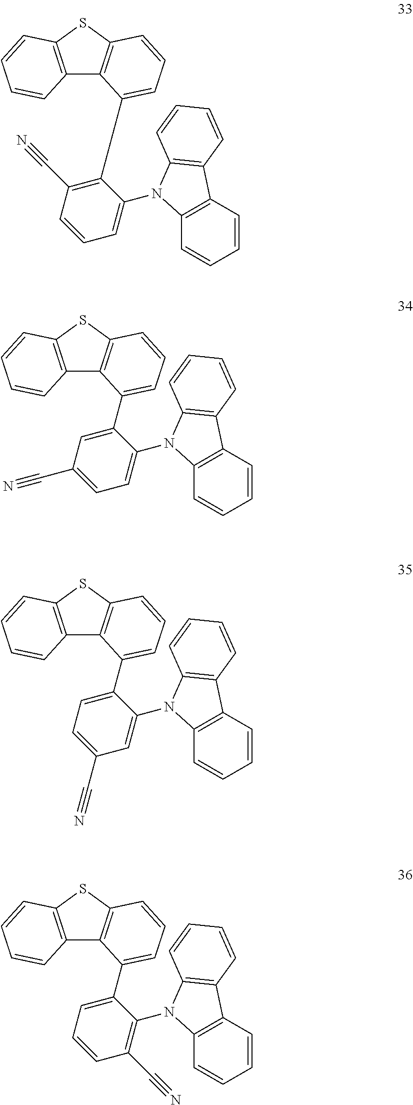 [SMILES: N#Cc1cccc(-n2c3ccccc3c3ccccc32)c1-c1cccc2sc3ccccc3c12 ; N#Cc1ccc(-n2c3ccccc3c3ccccc32)c(-c2cccc3sc4ccccc4c23)c1 ; N#Cc1ccc(-c2cccc3sc4ccccc4c23)c(-n2c3ccccc3c3ccccc32)c1 ; N#Cc1cccc(-c2cccc3sc4ccccc4c23)c1-n1c2ccccc2c2ccccc21]

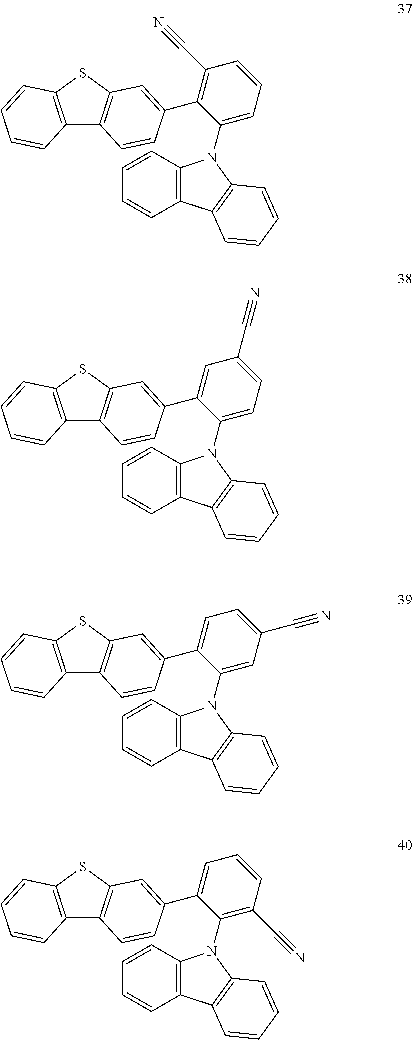 [SMILES: N#Cc1cccc(-n2c3ccccc3c3ccccc32)c1-c1ccc2c(c1)sc1ccccc12 ; N#Cc1ccc(-n2c3ccccc3c3ccccc32)c(-c2ccc3c(c2)sc2ccccc23)c1 ; N#Cc1ccc(-c2ccc3c(c2)sc2ccccc23)c(-n2c3ccccc3c3ccccc32)c1 ; N#Cc1cccc(-c2ccc3c(c2)sc2ccccc23)c1-n1c2ccccc2c2ccccc21]

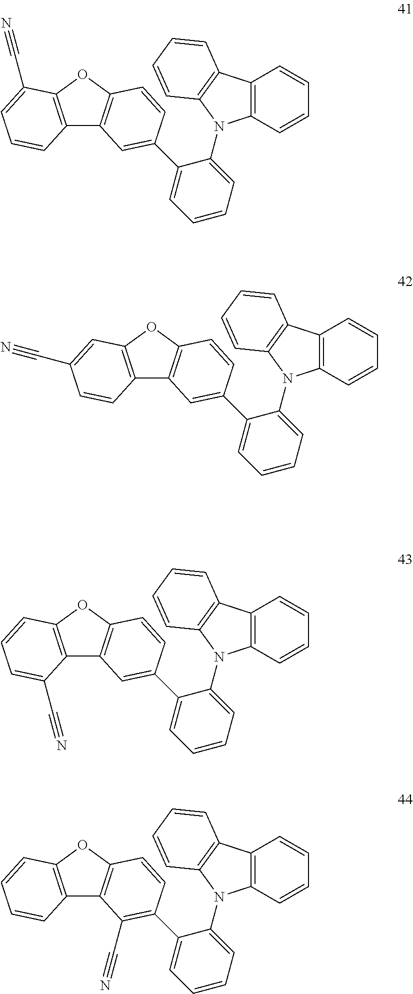 [SMILES: N#Cc1cccc2c1oc1ccc(-c3ccccc3-n3c4ccccc4c4ccccc43)cc12 ; N#Cc1ccc2c(c1)oc1ccc(-c3ccccc3-n3c4ccccc4c4ccccc43)cc12 ; N#Cc1cccc2oc3ccc(-c4ccccc4-n4c5ccccc5c5ccccc54)cc3c12 ; N#Cc1c(-c2ccccc2-n2c3ccccc3c3ccccc32)ccc2oc3ccccc3c12]

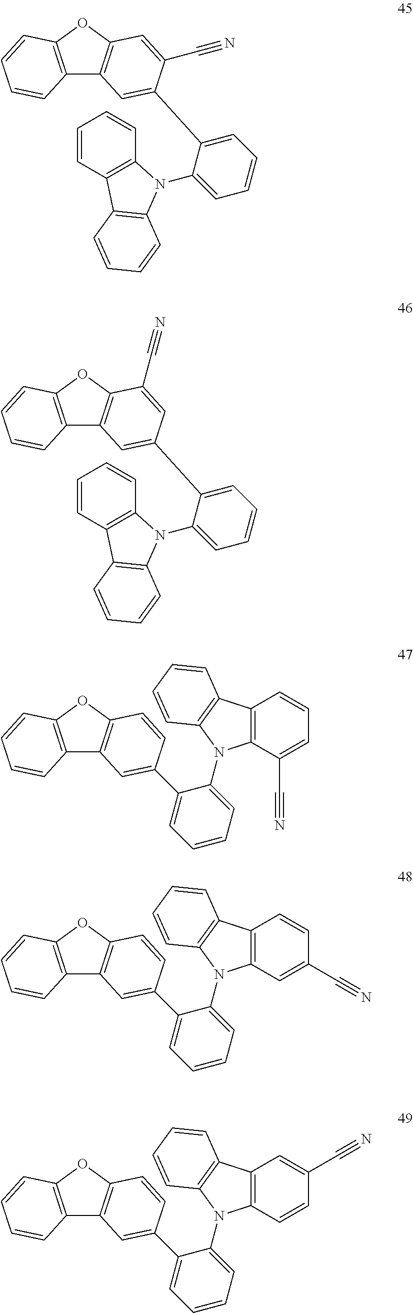

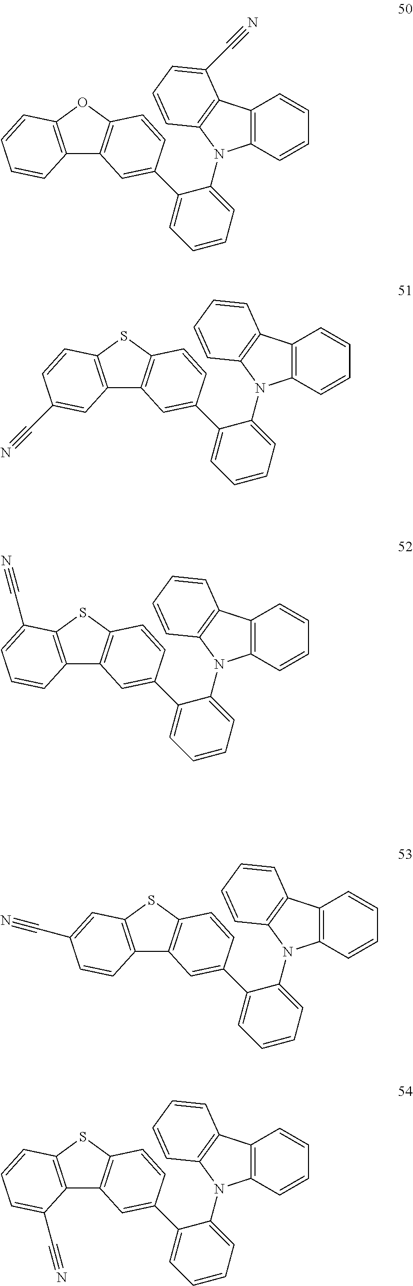 [SMILES: N#Cc1cccc2c1c1ccccc1n2-c1ccccc1-c1ccc2oc3ccccc3c2c1 ; N#Cc1ccc2sc3ccc(-c4ccccc4-n4c5ccccc5c5ccccc54)cc3c2c1 ; N#Cc1cccc2c1sc1ccc(-c3ccccc3-n3c4ccccc4c4ccccc43)cc12 ; N#Cc1ccc2c(c1)sc1ccc(-c3ccccc3-n3c4ccccc4c4ccccc43)cc12 ; N#Cc1cccc2sc3ccc(-c4ccccc4-n4c5ccccc5c5ccccc54)cc3c12]

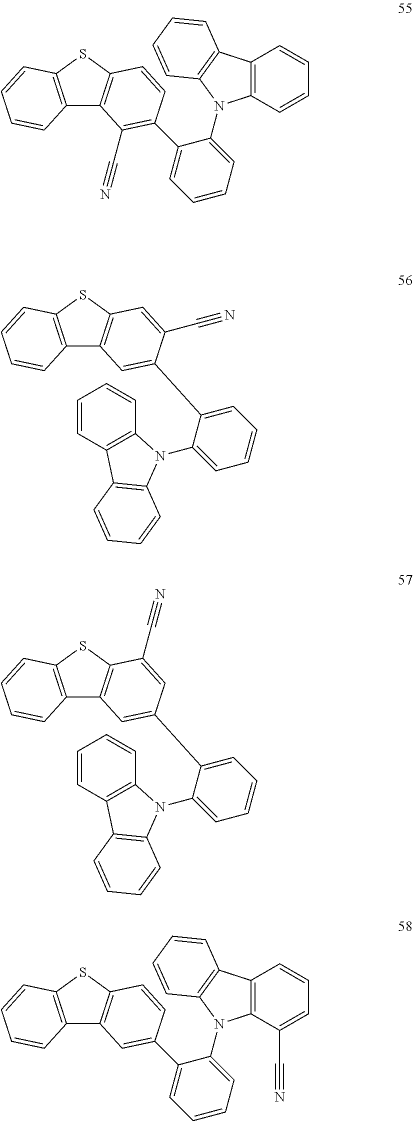 [SMILES: N#Cc1c(-c2ccccc2-n2c3ccccc3c3ccccc32)ccc2sc3ccccc3c12 ; N#Cc1cc2sc3ccccc3c2cc1-c1ccccc1-n1c2ccccc2c2ccccc21 ; N#Cc1cc(-c2ccccc2-n2c3ccccc3c3ccccc32)cc2c1sc1ccccc12 ; N#Cc1cccc2c3ccccc3n(-c3ccccc3-c3ccc4sc5ccccc5c4c3)c12]

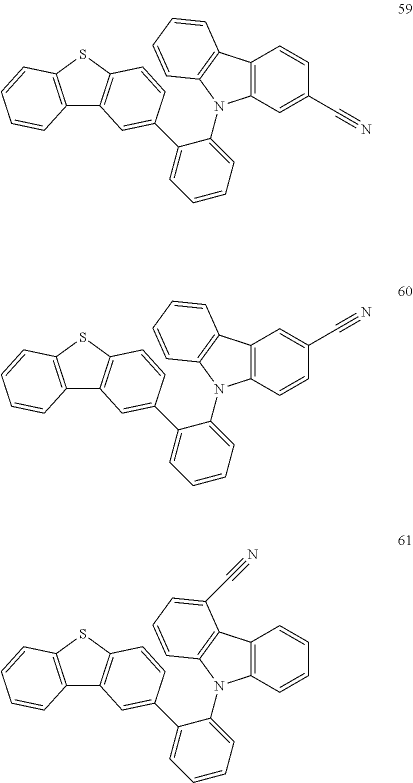 [SMILES: N#Cc1ccc2c3ccccc3n(-c3ccccc3-c3ccc4sc5ccccc5c4c3)c2c1 ; N#Cc1ccc2c(c1)c1ccccc1n2-c1ccccc1-c1ccc2sc3ccccc3c2c1 ; N#Cc1cccc2c1c1ccccc1n2-c1ccccc1-c1ccc2sc3ccccc3c2c1]

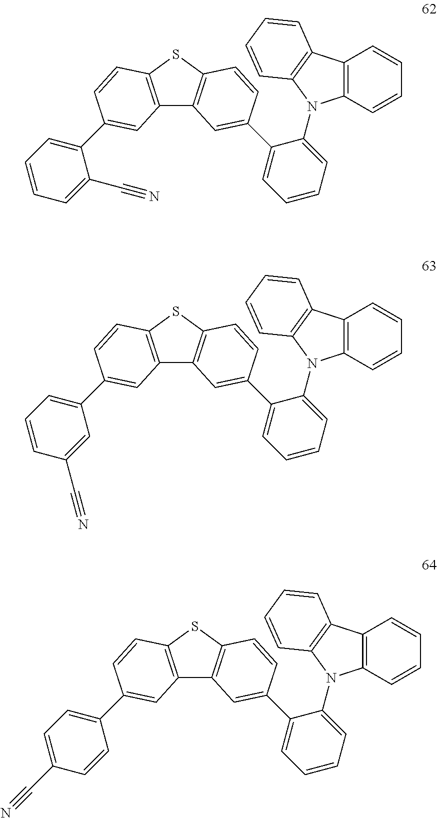 [SMILES: N#Cc1ccccc1-c1ccc2sc3ccc(-c4ccccc4-n4c5ccccc5c5ccccc54)cc3c2c1 ; N#Cc1cccc(-c2ccc3sc4ccc(-c5ccccc5-n5c6ccccc6c6ccccc65)cc4c3c2)c1 ; N#Cc1ccc(-c2ccc3sc4ccc(-c5ccccc5-n5c6ccccc6c6ccccc65)cc4c3c2)cc1]

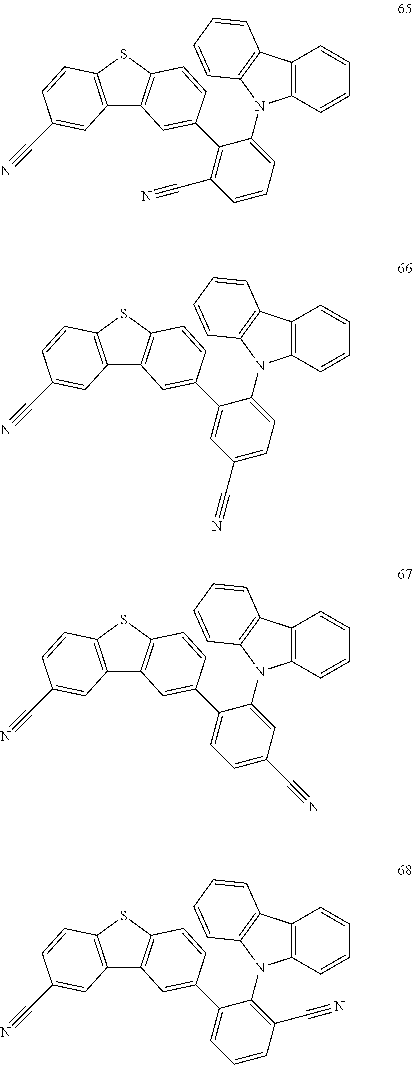 [SMILES: N#Cc1ccc2sc3ccc(-c4c(C#N)cccc4-n4c5ccccc5c5ccccc54)cc3c2c1 ; N#Cc1ccc(-n2c3ccccc3c3ccccc32)c(-c2ccc3sc4ccc(C#N)cc4c3c2)c1 ; N#Cc1ccc(-c2ccc3sc4ccc(C#N)cc4c3c2)c(-n2c3ccccc3c3ccccc32)c1 ; N#Cc1ccc2sc3ccc(-c4cccc(C#N)c4-n4c5ccccc5c5ccccc54)cc3c2c1]

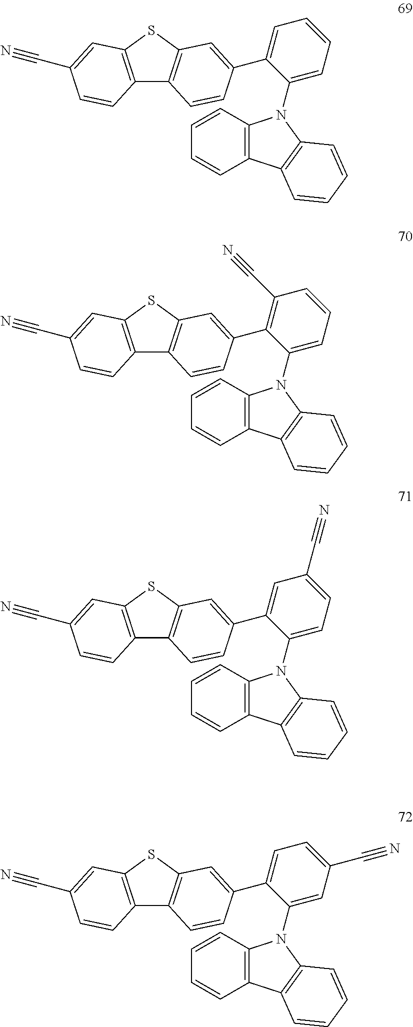

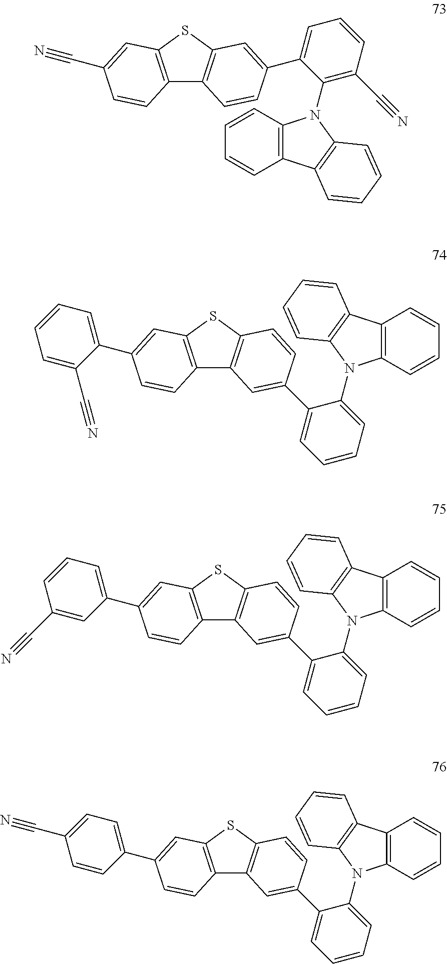 [SMILES: N#Cc1ccc2c(c1)sc1cc(-c3cccc(C#N)c3-n3c4ccccc4c4ccccc43)ccc12 ; N#Cc1ccccc1-c1ccc2c(c1)sc1ccc(-c3ccccc3-n3c4ccccc4c4ccccc43)cc12 ; N#Cc1cccc(-c2ccc3c(c2)sc2ccc(-c4ccccc4-n4c5ccccc5c5ccccc54)cc23)c1 ; N#Cc1ccc(-c2ccc3c(c2)sc2ccc(-c4ccccc4-n4c5ccccc5c5ccccc54)cc23)cc1]

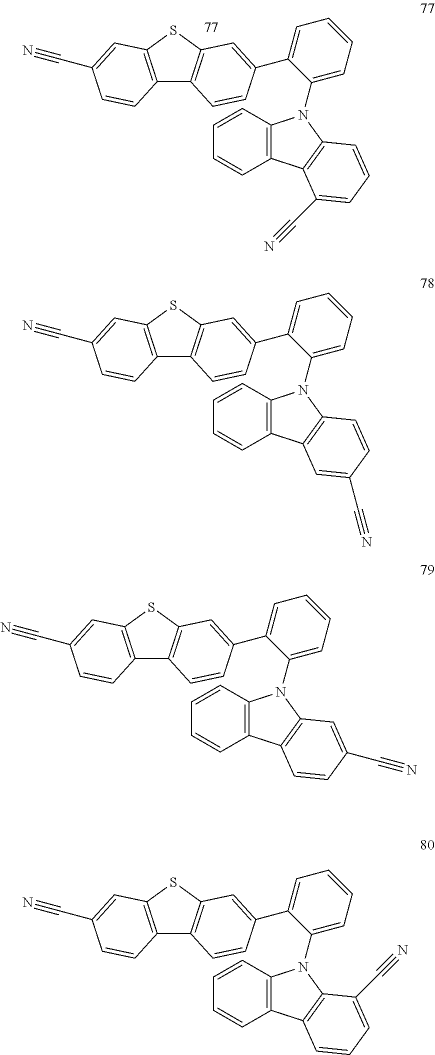 [SMILES: N#Cc1ccc2c(c1)sc1cc(-c3ccccc3-n3c4ccccc4c4c(C#N)cccc43)ccc12 ; N#Cc1ccc2c(c1)sc1cc(-c3ccccc3-n3c4ccccc4c4cc(C#N)ccc43)ccc12 ; N#Cc1ccc2c(c1)sc1cc(-c3ccccc3-n3c4ccccc4c4ccc(C#N)cc43)ccc12 ; N#Cc1ccc2c(c1)sc1cc(-c3ccccc3-n3c4ccccc4c4cccc(C#N)c43)ccc12]

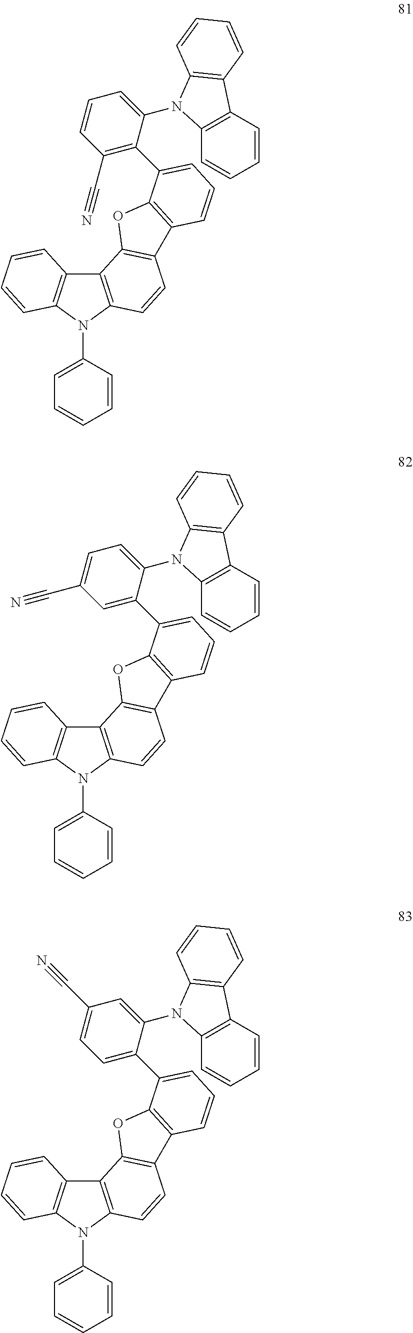 [SMILES: N#Cc1cccc(-n2c3ccccc3c3ccccc32)c1-c1cccc2c1oc1c2ccc2c1c1ccccc1n2-c1ccccc1 ; N#Cc1ccc(-n2c3ccccc3c3ccccc32)c(-c2cccc3c2oc2c3ccc3c2c2ccccc2n3-c2ccccc2)c1 ; N#Cc1ccc(-c2cccc3c2oc2c3ccc3c2c2ccccc2n3-c2ccccc2)c(-n2c3ccccc3c3ccccc32)c1]

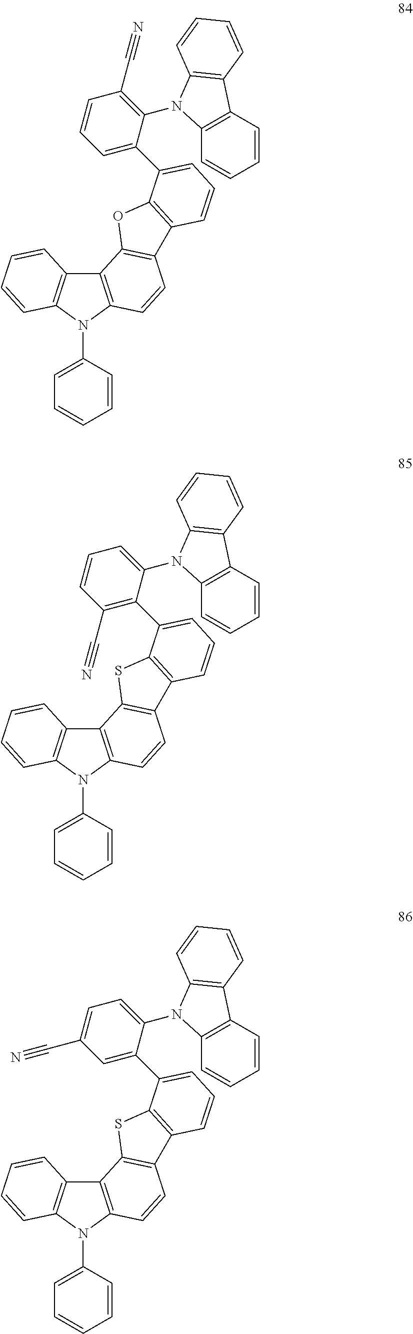 [SMILES: N#Cc1cccc(-c2cccc3c2oc2c3ccc3c2c2ccccc2n3-c2ccccc2)c1-n1c2ccccc2c2ccccc21 ; N#Cc1cccc(-n2c3ccccc3c3ccccc32)c1-c1cccc2c1sc1c2ccc2c1c1ccccc1n2-c1ccccc1 ; N#Cc1ccc(-n2c3ccccc3c3ccccc32)c(-c2cccc3c2sc2c3ccc3c2c2ccccc2n3-c2ccccc2)c1]

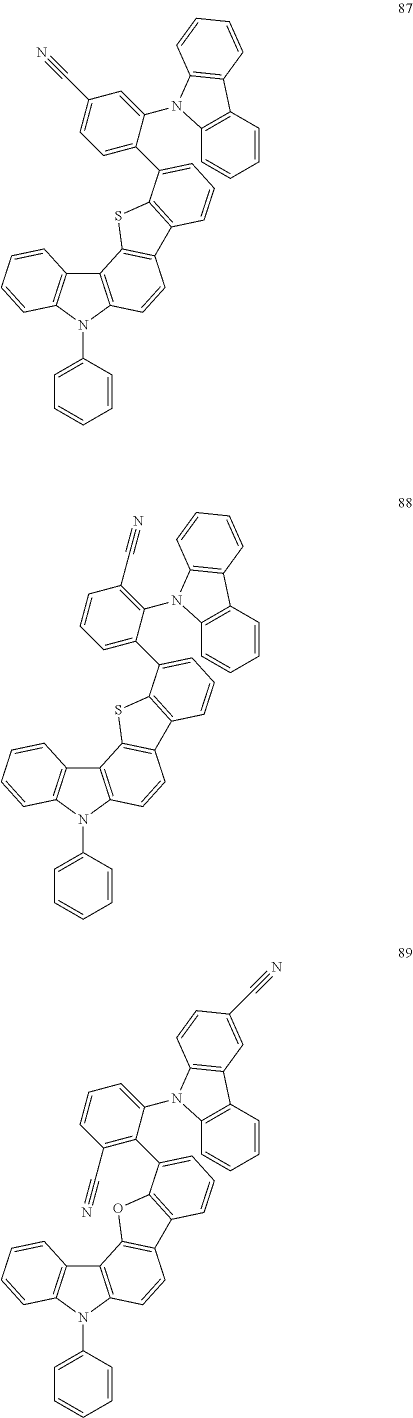 [SMILES: N#Cc1ccc(-c2cccc3c2sc2c3ccc3c2c2ccccc2n3-c2ccccc2)c(-n2c3ccccc3c3ccccc32)c1 ; N#Cc1cccc(-c2cccc3c2sc2c3ccc3c2c2ccccc2n3-c2ccccc2)c1-n1c2ccccc2c2ccccc21 ; N#Cc1ccc2c(c1)c1ccccc1n2-c1cccc(C#N)c1-c1cccc2c1oc1c2ccc2c1c1ccccc1n2-c1ccccc1]

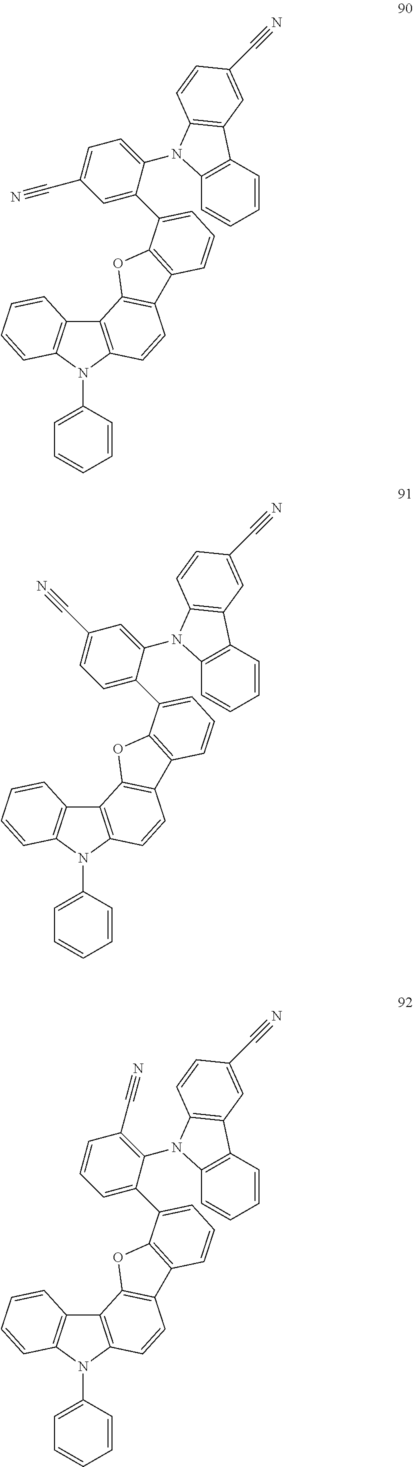 [SMILES: N#Cc1ccc(-n2c3ccccc3c3cc(C#N)ccc32)c(-c2cccc3c2oc2c3ccc3c2c2ccccc2n3-c2ccccc2)c1 ; N#Cc1ccc(-c2cccc3c2oc2c3ccc3c2c2ccccc2n3-c2ccccc2)c(-n2c3ccccc3c3cc(C#N)ccc32)c1 ; N#Cc1ccc2c(c1)c1ccccc1n2-c1c(C#N)cccc1-c1cccc2c1oc1c2ccc2c1c1ccccc1n2-c1ccccc1]

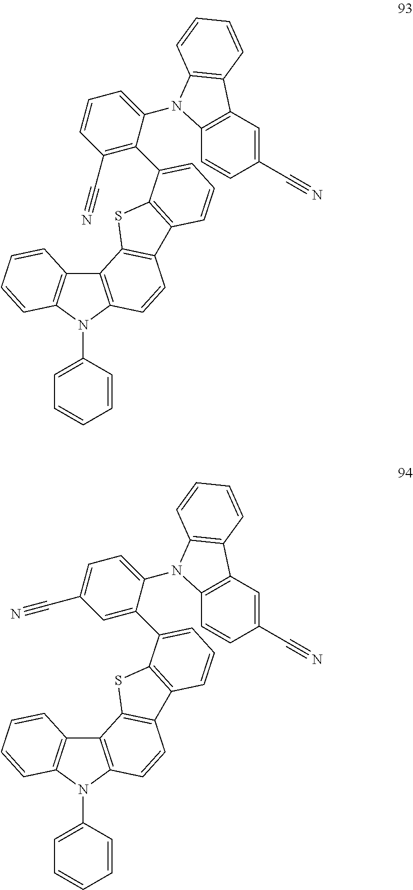 [SMILES: N#Cc1ccc2c(c1)c1ccccc1n2-c1cccc(C#N)c1-c1cccc2c1sc1c2ccc2c1c1ccccc1n2-c1ccccc1 ; N#Cc1ccc(-n2c3ccccc3c3cc(C#N)ccc32)c(-c2cccc3c2sc2c3ccc3c2c2ccccc2n3-c2ccccc2)c1]

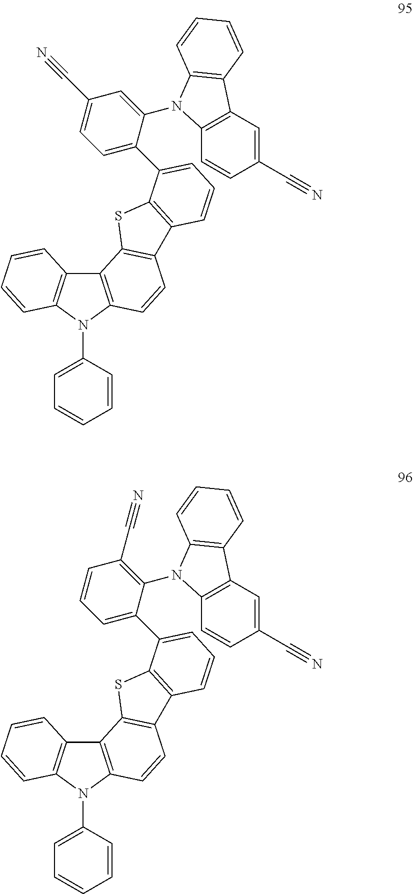 [SMILES: N#Cc1ccc(-c2cccc3c2sc2c3ccc3c2c2ccccc2n3-c2ccccc2)c(-n2c3ccccc3c3cc(C#N)ccc32)c1 ; N#Cc1ccc2c(c1)c1ccccc1n2-c1c(C#N)cccc1-c1cccc2c1sc1c2ccc2c1c1ccccc1n2-c1ccccc1]

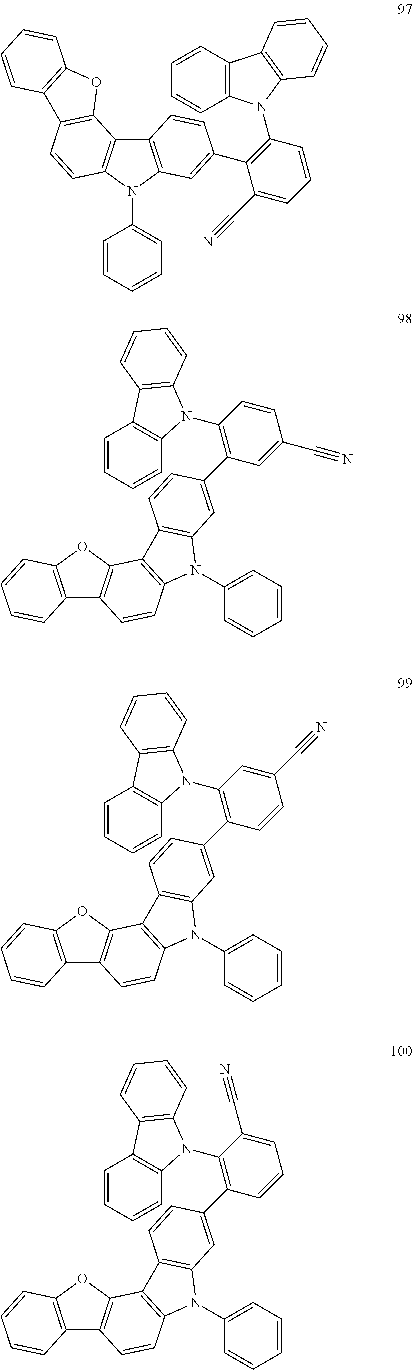

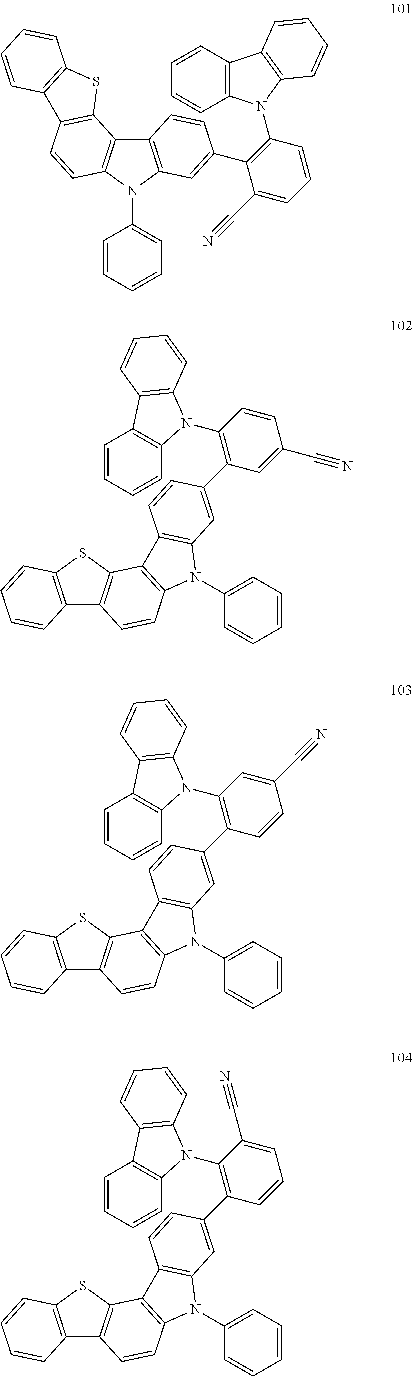 [SMILES: N#Cc1cccc(-n2c3ccccc3c3ccccc32)c1-c1ccc2c3c4sc5ccccc5c4ccc3n(-c3ccccc3)c2c1 ; N#Cc1ccc(-n2c3ccccc3c3ccccc32)c(-c2ccc3c4c5sc6ccccc6c5ccc4n(-c4ccccc4)c3c2)c1 ; N#Cc1ccc(-c2ccc3c4c5sc6ccccc6c5ccc4n(-c4ccccc4)c3c2)c(-n2c3ccccc3c3ccccc32)c1 ; N#Cc1cccc(-c2ccc3c4c5sc6ccccc6c5ccc4n(-c4ccccc4)c3c2)c1-n1c2ccccc2c2ccccc21]

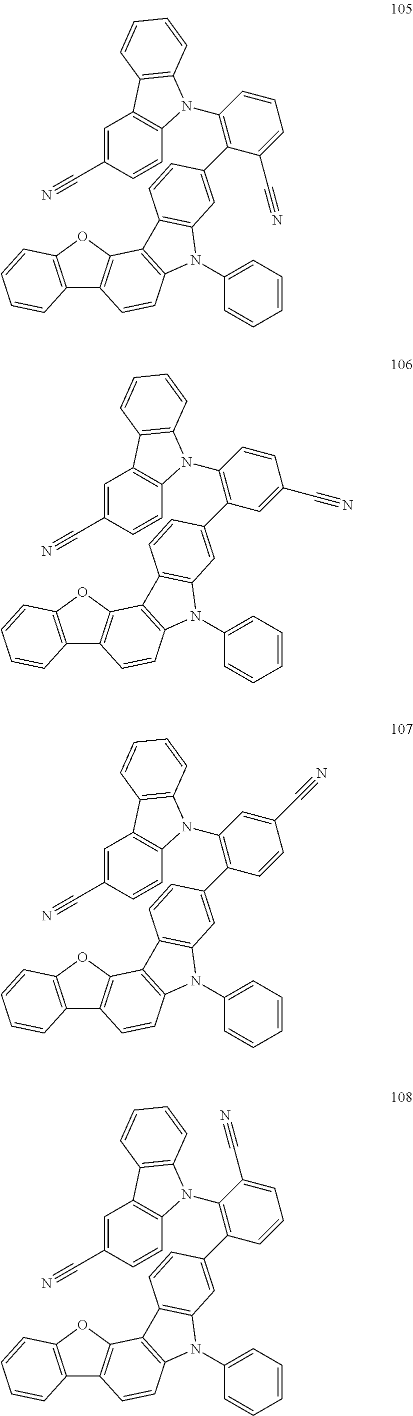 [SMILES: N#Cc1ccc2c(c1)c1ccccc1n2-c1cccc(C#N)c1-c1ccc2c3c4oc5ccccc5c4ccc3n(-c3ccccc3)c2c1 ; N#Cc1ccc(-n2c3ccccc3c3cc(C#N)ccc32)c(-c2ccc3c4c5oc6ccccc6c5ccc4n(-c4ccccc4)c3c2)c1 ; N#Cc1ccc(-c2ccc3c4c5oc6ccccc6c5ccc4n(-c4ccccc4)c3c2)c(-n2c3ccccc3c3cc(C#N)ccc32)c1 ; N#Cc1ccc2c(c1)c1ccccc1n2-c1c(C#N)cccc1-c1ccc2c3c4oc5ccccc5c4ccc3n(-c3ccccc3)c2c1]

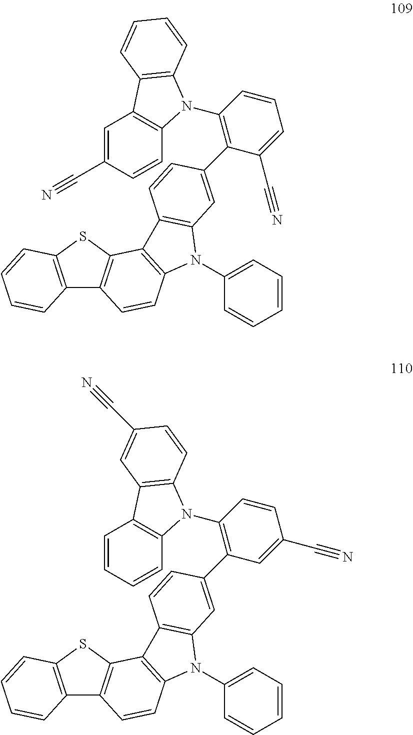 [SMILES: N#Cc1ccc2c(c1)c1ccccc1n2-c1cccc(C#N)c1-c1ccc2c3c4sc5ccccc5c4ccc3n(-c3ccccc3)c2c1 ; N#Cc1ccc(-n2c3ccccc3c3cc(C#N)ccc32)c(-c2ccc3c4c5sc6ccccc6c5ccc4n(-c4ccccc4)c3c2)c1]

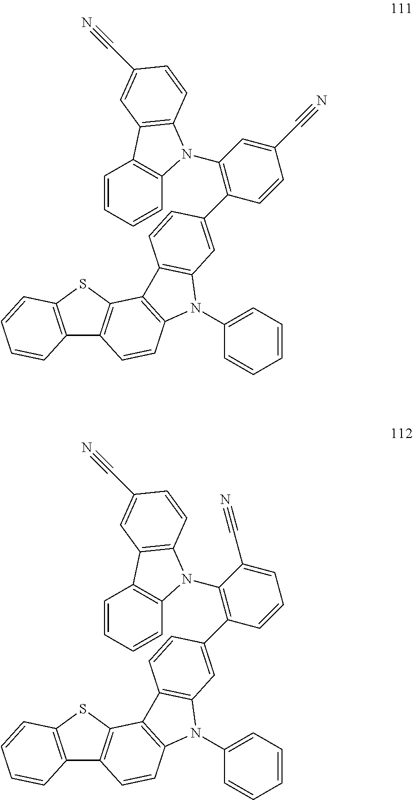 [SMILES: N#Cc1ccc(-c2ccc3c4c5sc6ccccc6c5ccc4n(-c4ccccc4)c3c2)c(-n2c3ccccc3c3cc(C#N)ccc32)c1 ; N#Cc1ccc2c(c1)c1ccccc1n2-c1c(C#N)cccc1-c1ccc2c3c4sc5ccccc5c4ccc3n(-c3ccccc3)c2c1]

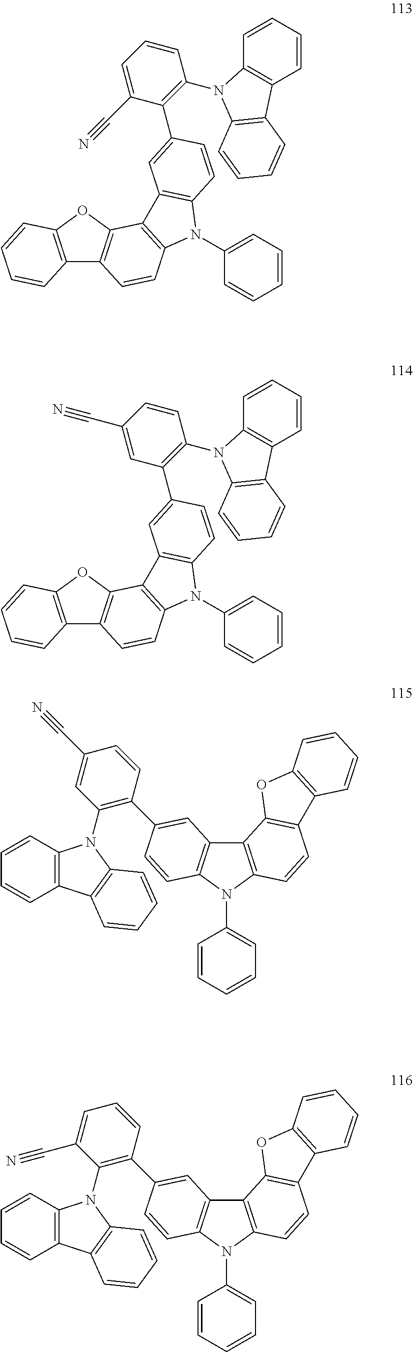 [SMILES: N#Cc1cccc(-n2c3ccccc3c3ccccc32)c1-c1ccc2c(c1)c1c3oc4ccccc4c3ccc1n2-c1ccccc1 ; N#Cc1ccc(-n2c3ccccc3c3ccccc32)c(-c2ccc3c(c2)c2c4oc5ccccc5c4ccc2n3-c2ccccc2)c1 ; N#Cc1ccc(-c2ccc3c(c2)c2c4oc5ccccc5c4ccc2n3-c2ccccc2)c(-n2c3ccccc3c3ccccc32)c1 ; N#Cc1cccc(-c2ccc3c(c2)c2c4oc5ccccc5c4ccc2n3-c2ccccc2)c1-n1c2ccccc2c2ccccc21]

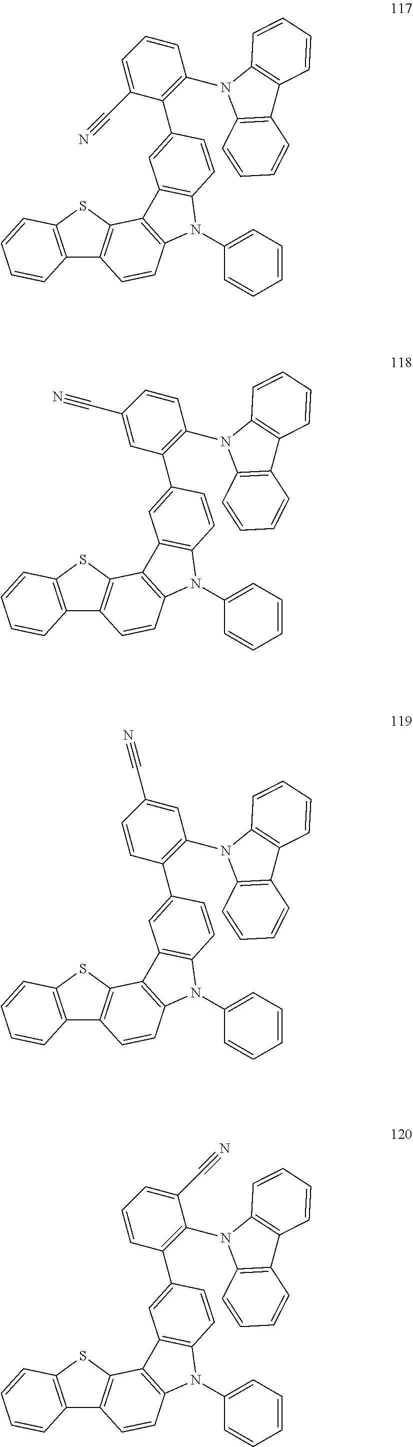 [SMILES: N#Cc1cccc(-n2c3ccccc3c3ccccc32)c1-c1ccc2c(c1)c1c3sc4ccccc4c3ccc1n2-c1ccccc1 ; N#Cc1ccc(-n2c3ccccc3c3ccccc32)c(-c2ccc3c(c2)c2c4sc5ccccc5c4ccc2n3-c2ccccc2)c1 ; N#Cc1ccc(-c2ccc3c(c2)c2c4sc5ccccc5c4ccc2n3-c2ccccc2)c(-n2c3ccccc3c3ccccc32)c1 ; N#Cc1cccc(-c2ccc3c(c2)c2c4sc5ccccc5c4ccc2n3-c2ccccc2)c1-n1c2ccccc2c2ccccc21]

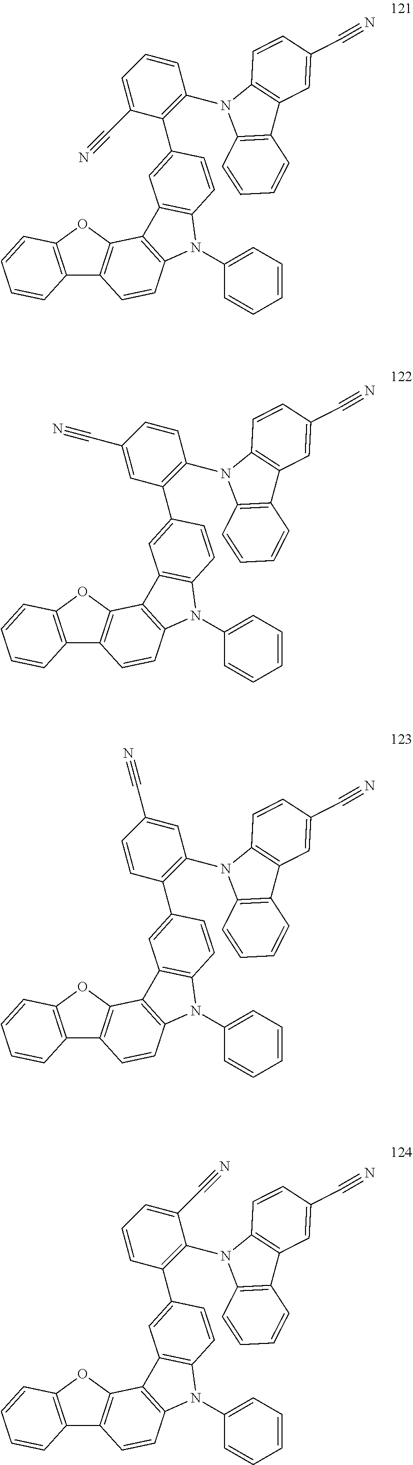 [SMILES: N#Cc1ccc2c(c1)c1ccccc1n2-c1cccc(C#N)c1-c1ccc2c(c1)c1c3oc4ccccc4c3ccc1n2-c1ccccc1 ; N#Cc1ccc(-n2c3ccccc3c3cc(C#N)ccc32)c(-c2ccc3c(c2)c2c4oc5ccccc5c4ccc2n3-c2ccccc2)c1 ; N#Cc1ccc(-c2ccc3c(c2)c2c4oc5ccccc5c4ccc2n3-c2ccccc2)c(-n2c3ccccc3c3cc(C#N)ccc32)c1 ; N#Cc1ccc2c(c1)c1ccccc1n2-c1c(C#N)cccc1-c1ccc2c(c1)c1c3oc4ccccc4c3ccc1n2-c1ccccc1]

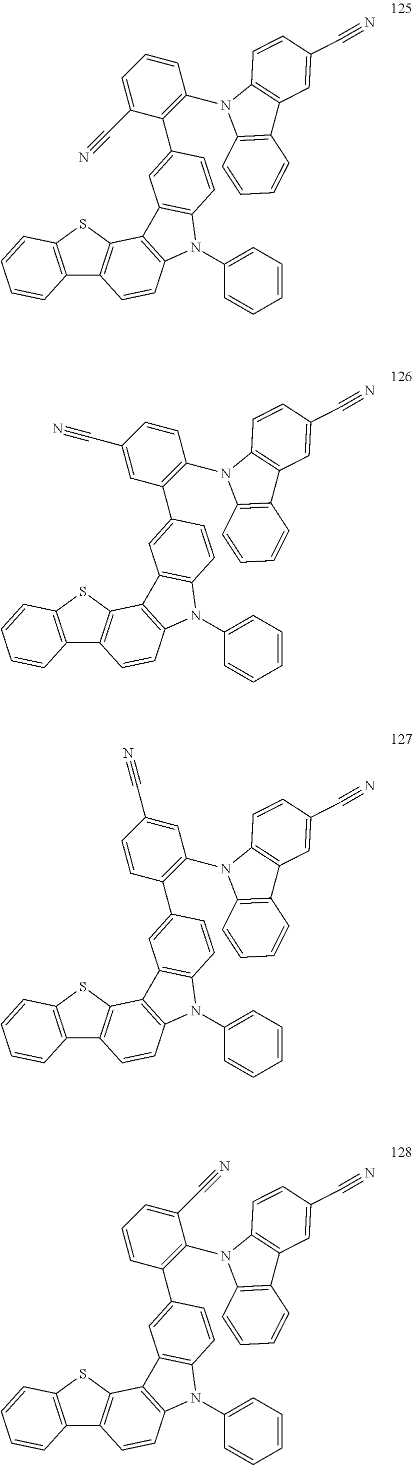 [SMILES: N#Cc1ccc2c(c1)c1ccccc1n2-c1cccc(C#N)c1-c1ccc2c(c1)c1c3sc4ccccc4c3ccc1n2-c1ccccc1 ; N#Cc1ccc(-n2c3ccccc3c3cc(C#N)ccc32)c(-c2ccc3c(c2)c2c4sc5ccccc5c4ccc2n3-c2ccccc2)c1 ; N#Cc1ccc(-c2ccc3c(c2)c2c4sc5ccccc5c4ccc2n3-c2ccccc2)c(-n2c3ccccc3c3cc(C#N)ccc32)c1 ; N#Cc1ccc2c(c1)c1ccccc1n2-c1c(C#N)cccc1-c1ccc2c(c1)c1c3sc4ccccc4c3ccc1n2-c1ccccc1]

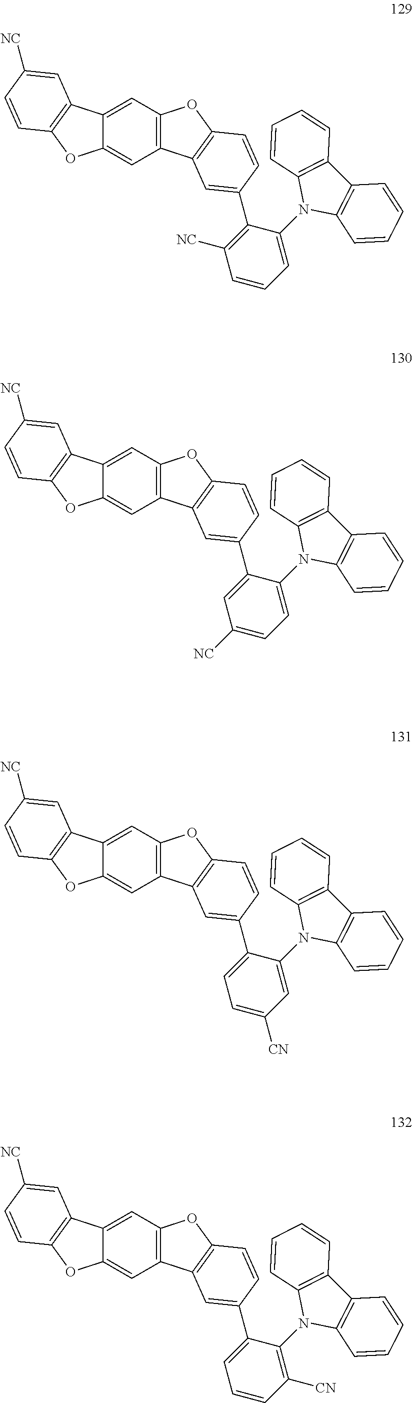 [SMILES: N#Cc1ccc2oc3cc4c(cc3c2c1)oc1ccc(-c2c(C#N)cccc2-n2c3ccccc3c3ccccc32)cc14 ; N#Cc1ccc(-n2c3ccccc3c3ccccc32)c(-c2ccc3oc4cc5c(cc4c3c2)oc2ccc(C#N)cc25)c1 ; N#Cc1ccc(-c2ccc3oc4cc5c(cc4c3c2)oc2ccc(C#N)cc25)c(-n2c3ccccc3c3ccccc32)c1 ; N#Cc1ccc2oc3cc4c(cc3c2c1)oc1ccc(-c2cccc(C#N)c2-n2c3ccccc3c3ccccc32)cc14]

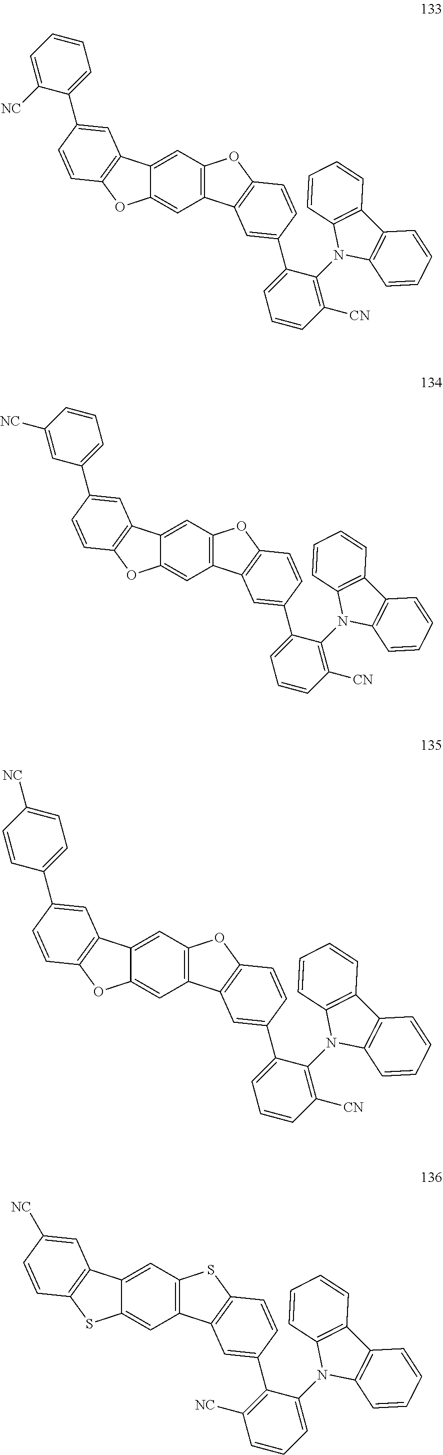 [SMILES: N#Cc1ccccc1-c1ccc2oc3cc4c(cc3c2c1)oc1ccc(-c2cccc(C#N)c2-n2c3ccccc3c3ccccc32)cc14 ; N#Cc1cccc(-c2ccc3oc4cc5c(cc4c3c2)oc2ccc(-c3cccc(C#N)c3-n3c4ccccc4c4ccccc43)cc25)c1 ; N#Cc1ccc(-c2ccc3oc4cc5c(cc4c3c2)oc2ccc(-c3cccc(C#N)c3-n3c4ccccc4c4ccccc43)cc25)cc1 ; N#Cc1ccc2sc3cc4c(cc3c2c1)sc1ccc(-c2c(C#N)cccc2-n2c3ccccc3c3ccccc32)cc14]

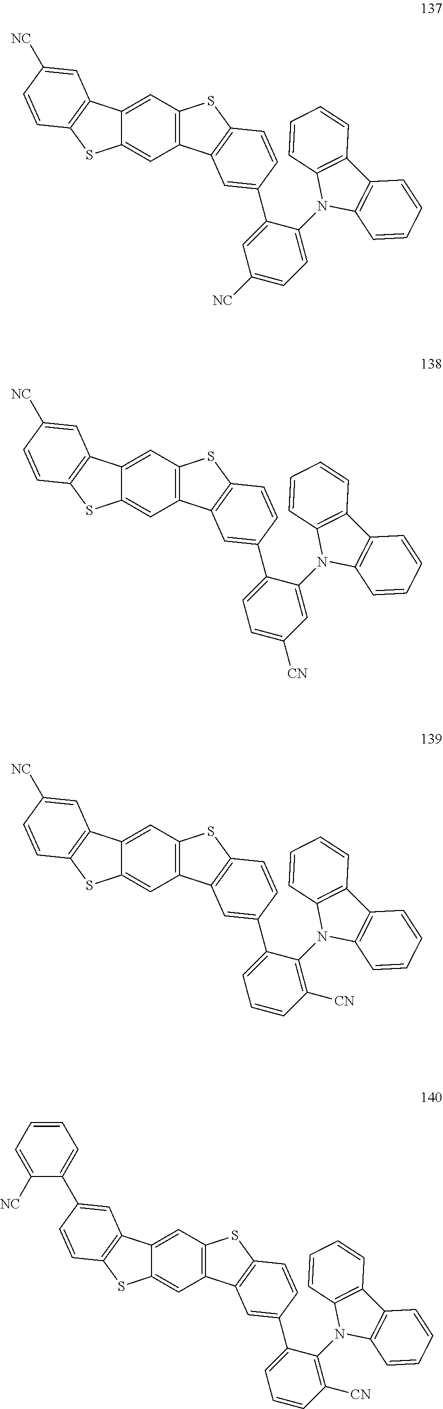 [SMILES: N#Cc1ccc(-n2c3ccccc3c3ccccc32)c(-c2ccc3sc4cc5c(cc4c3c2)sc2ccc(C#N)cc25)c1 ; N#Cc1ccc(-c2ccc3sc4cc5c(cc4c3c2)sc2ccc(C#N)cc25)c(-n2c3ccccc3c3ccccc32)c1 ; N#Cc1ccc2sc3cc4c(cc3c2c1)sc1ccc(-c2cccc(C#N)c2-n2c3ccccc3c3ccccc32)cc14 ; N#Cc1ccccc1-c1ccc2sc3cc4c(cc3c2c1)sc1ccc(-c2cccc(C#N)c2-n2c3ccccc3c3ccccc32)cc14]

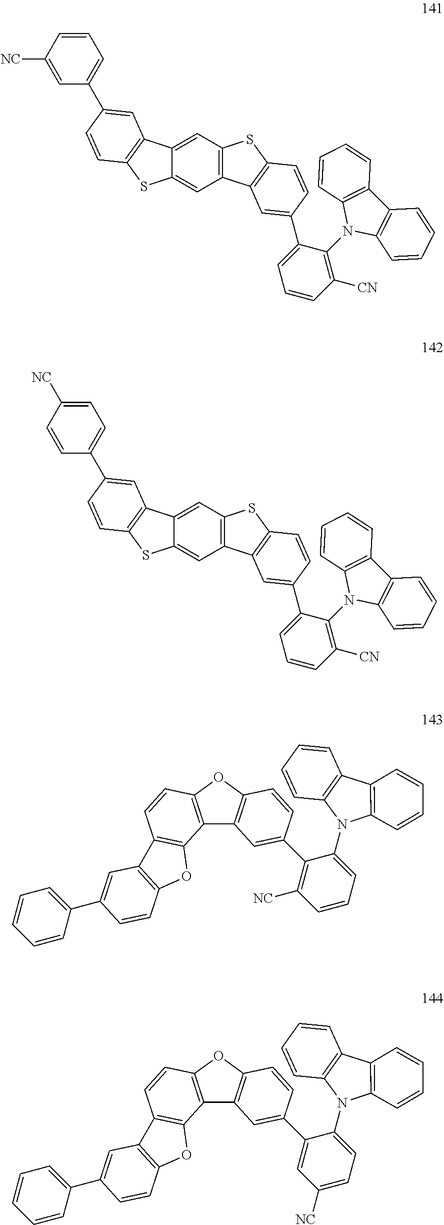 [SMILES: N#Cc1cccc(-c2ccc3sc4cc5c(cc4c3c2)sc2ccc(-c3cccc(C#N)c3-n3c4ccccc4c4ccccc43)cc25)c1 ; N#Cc1ccc(-c2ccc3sc4cc5c(cc4c3c2)sc2ccc(-c3cccc(C#N)c3-n3c4ccccc4c4ccccc43)cc25)cc1 ; N#Cc1cccc(-n2c3ccccc3c3ccccc32)c1-c1ccc2oc3ccc4c5cc(-c6ccccc6)ccc5oc4c3c2c1 ; N#Cc1ccc(-n2c3ccccc3c3ccccc32)c(-c2ccc3oc4ccc5c6cc(-c7ccccc7)ccc6oc5c4c3c2)c1]

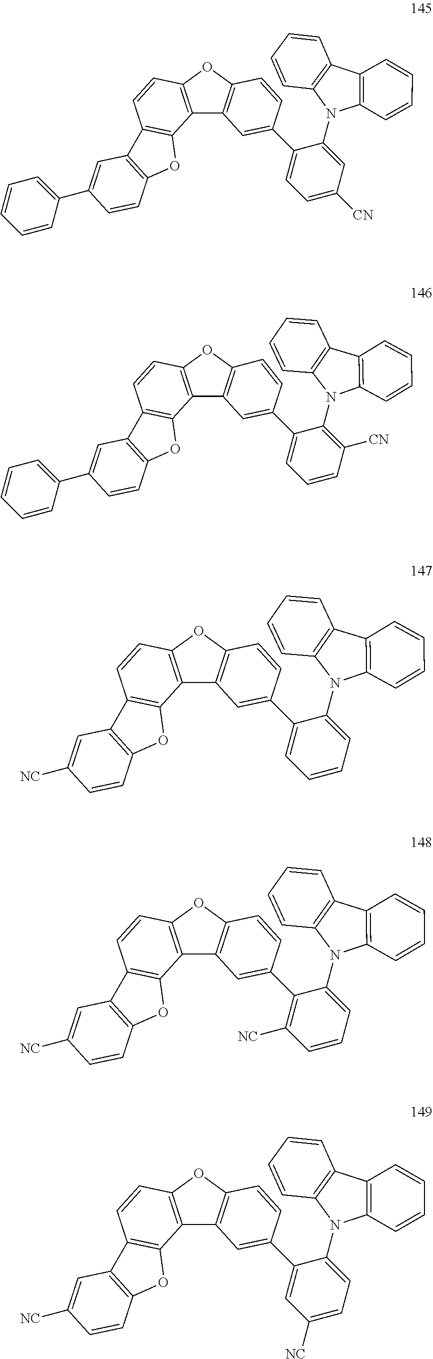 [SMILES: N#Cc1ccc(-c2ccc3oc4ccc5c6cc(-c7ccccc7)ccc6oc5c4c3c2)c(-n2c3ccccc3c3ccccc32)c1 ; N#Cc1cccc(-c2ccc3oc4ccc5c6cc(-c7ccccc7)ccc6oc5c4c3c2)c1-n1c2ccccc2c2ccccc21 ; N#Cc1ccc2oc3c(ccc4oc5ccc(-c6ccccc6-n6c7ccccc7c7ccccc76)cc5c43)c2c1 ; N#Cc1ccc2oc3c(ccc4oc5ccc(-c6c(C#N)cccc6-n6c7ccccc7c7ccccc76)cc5c43)c2c1 ; N#Cc1ccc(-n2c3ccccc3c3ccccc32)c(-c2ccc3oc4ccc5c6cc(C#N)ccc6oc5c4c3c2)c1]

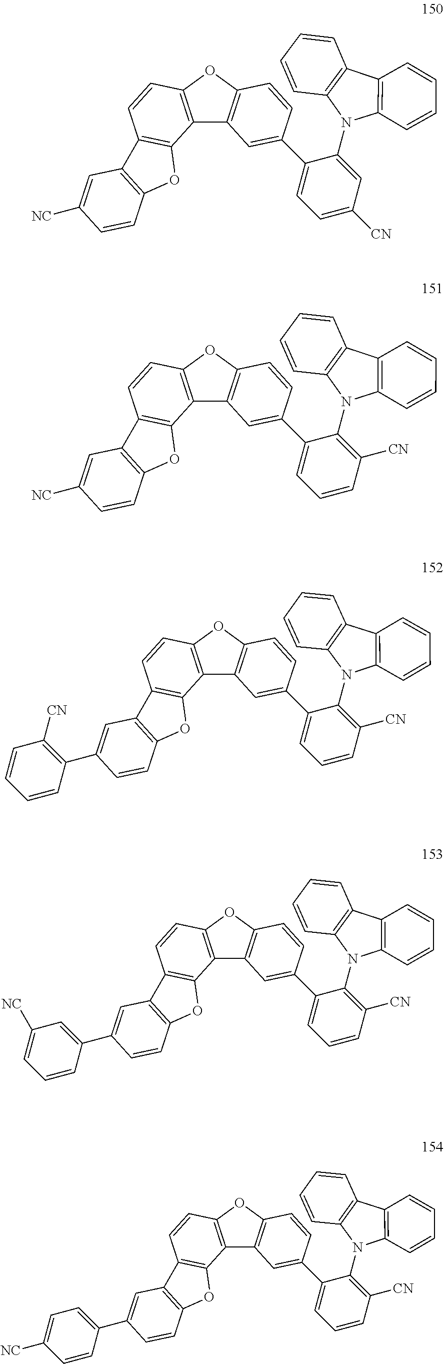 [SMILES: N#Cc1ccc(-c2ccc3oc4ccc5c6cc(C#N)ccc6oc5c4c3c2)c(-n2c3ccccc3c3ccccc32)c1 ; N#Cc1ccc2oc3c(ccc4oc5ccc(-c6cccc(C#N)c6-n6c7ccccc7c7ccccc76)cc5c43)c2c1 ; N#Cc1ccccc1-c1ccc2oc3c(ccc4oc5ccc(-c6cccc(C#N)c6-n6c7ccccc7c7ccccc76)cc5c43)c2c1 ; N#Cc1cccc(-c2ccc3oc4c(ccc5oc6ccc(-c7cccc(C#N)c7-n7c8ccccc8c8ccccc87)cc6c54)c3c2)c1 ; N#Cc1ccc(-c2ccc3oc4c(ccc5oc6ccc(-c7cccc(C#N)c7-n7c8ccccc8c8ccccc87)cc6c54)c3c2)cc1]

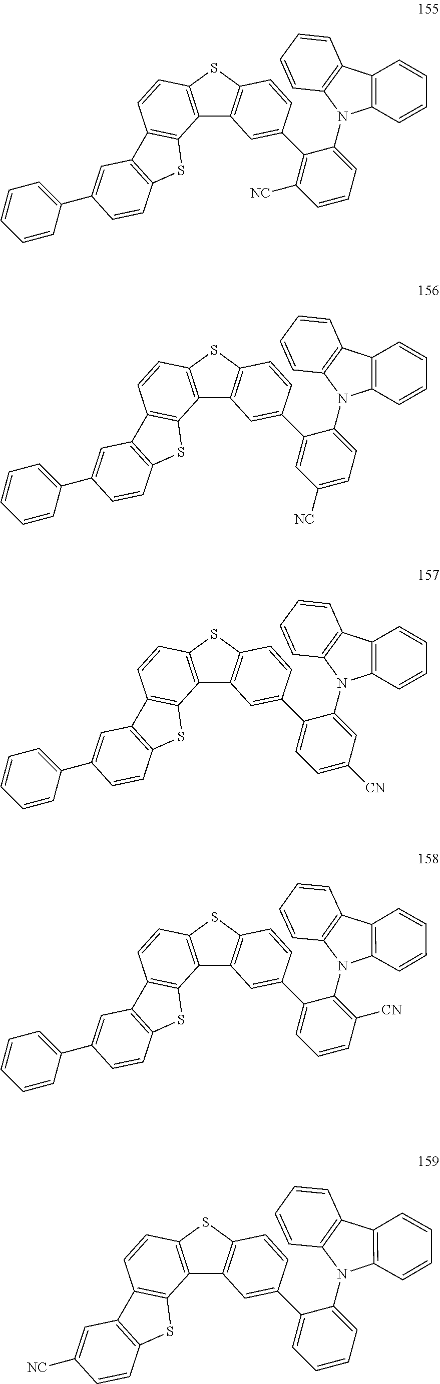 [SMILES: N#Cc1cccc(-n2c3ccccc3c3ccccc32)c1-c1ccc2sc3ccc4c5cc(-c6ccccc6)ccc5sc4c3c2c1 ; N#Cc1ccc(-n2c3ccccc3c3ccccc32)c(-c2ccc3sc4ccc5c6cc(-c7ccccc7)ccc6sc5c4c3c2)c1 ; N#Cc1ccc(-c2ccc3sc4ccc5c6cc(-c7ccccc7)ccc6sc5c4c3c2)c(-n2c3ccccc3c3ccccc32)c1 ; N#Cc1cccc(-c2ccc3sc4ccc5c6cc(-c7ccccc7)ccc6sc5c4c3c2)c1-n1c2ccccc2c2ccccc21 ; N#Cc1ccc2sc3c(ccc4sc5ccc(-c6ccccc6-n6c7ccccc7c7ccccc76)cc5c43)c2c1]

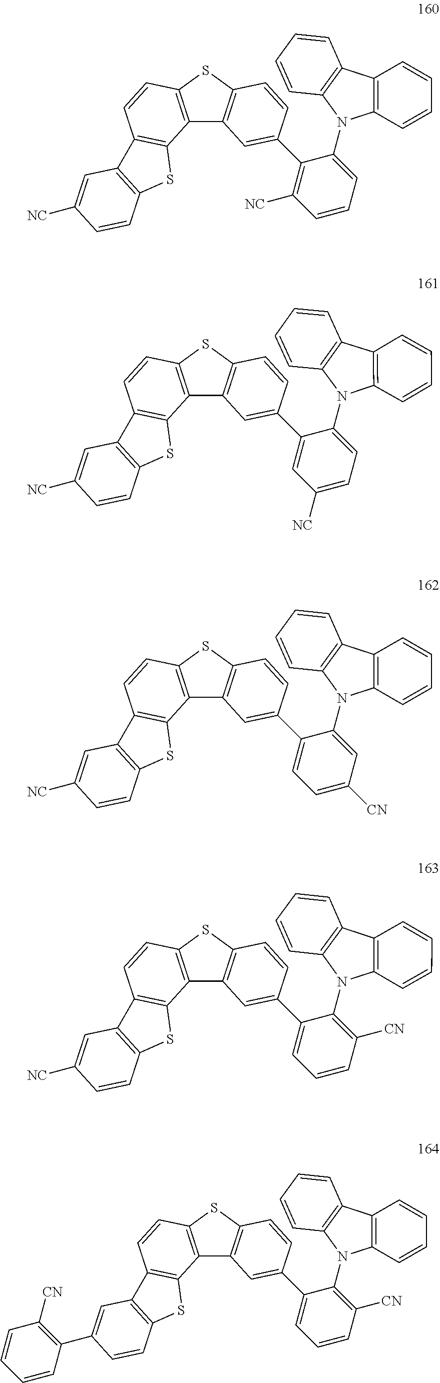 [SMILES: N#Cc1ccc2sc3c(ccc4sc5ccc(-c6c(C#N)cccc6-n6c7ccccc7c7ccccc76)cc5c43)c2c1 ; N#Cc1ccc(-n2c3ccccc3c3ccccc32)c(-c2ccc3sc4ccc5c6cc(C#N)ccc6sc5c4c3c2)c1 ; N#Cc1ccc(-c2ccc3sc4ccc5c6cc(C#N)ccc6sc5c4c3c2)c(-n2c3ccccc3c3ccccc32)c1 ; N#Cc1ccc2sc3c(ccc4sc5ccc(-c6cccc(C#N)c6-n6c7ccccc7c7ccccc76)cc5c43)c2c1 ; N#Cc1ccccc1-c1ccc2sc3c(ccc4sc5ccc(-c6cccc(C#N)c6-n6c7ccccc7c7ccccc76)cc5c43)c2c1]

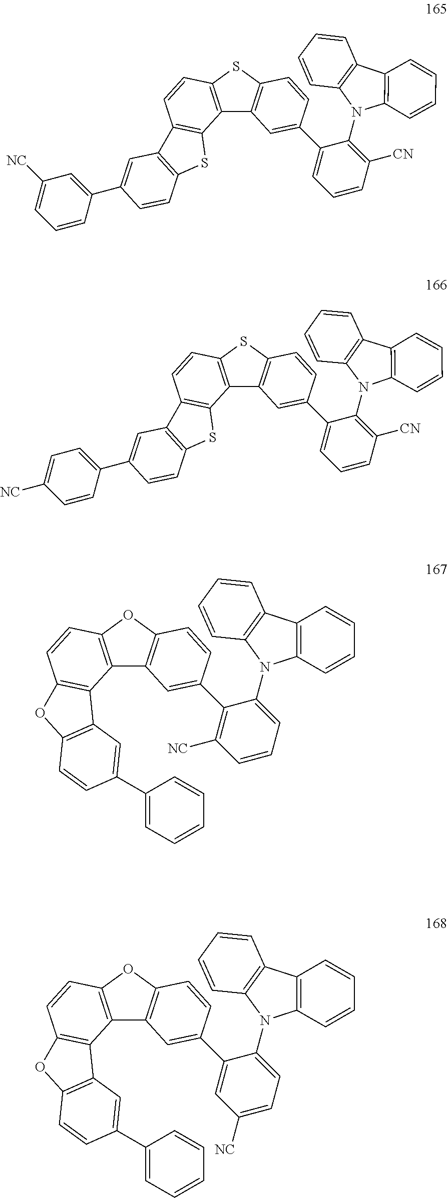 [SMILES: N#Cc1cccc(-c2ccc3sc4c(ccc5sc6ccc(-c7cccc(C#N)c7-n7c8ccccc8c8ccccc87)cc6c54)c3c2)c1 ; N#Cc1ccc(-c2ccc3sc4c(ccc5sc6ccc(-c7cccc(C#N)c7-n7c8ccccc8c8ccccc87)cc6c54)c3c2)cc1 ; N#Cc1cccc(-n2c3ccccc3c3ccccc32)c1-c1ccc2oc3ccc4oc5ccc(-c6ccccc6)cc5c4c3c2c1 ; N#Cc1ccc(-n2c3ccccc3c3ccccc32)c(-c2ccc3oc4ccc5oc6ccc(-c7ccccc7)cc6c5c4c3c2)c1]

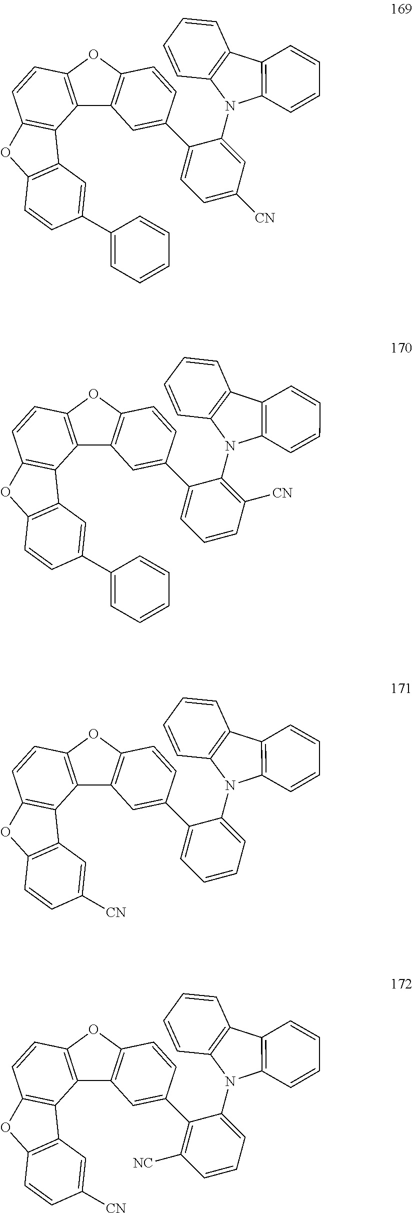 [SMILES: N#Cc1ccc(-c2ccc3oc4ccc5oc6ccc(-c7ccccc7)cc6c5c4c3c2)c(-n2c3ccccc3c3ccccc32)c1 ; N#Cc1cccc(-c2ccc3oc4ccc5oc6ccc(-c7ccccc7)cc6c5c4c3c2)c1-n1c2ccccc2c2ccccc21 ; N#Cc1ccc2oc3ccc4oc5ccc(-c6ccccc6-n6c7ccccc7c7ccccc76)cc5c4c3c2c1 ; N#Cc1ccc2oc3ccc4oc5ccc(-c6c(C#N)cccc6-n6c7ccccc7c7ccccc76)cc5c4c3c2c1]

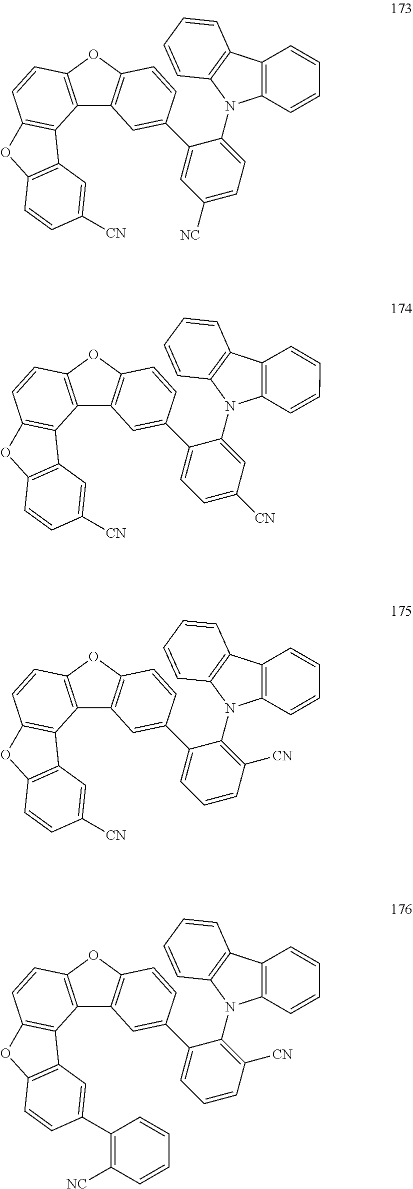 [SMILES: N#Cc1ccc(-n2c3ccccc3c3ccccc32)c(-c2ccc3oc4ccc5oc6ccc(C#N)cc6c5c4c3c2)c1 ; N#Cc1ccc(-c2ccc3oc4ccc5oc6ccc(C#N)cc6c5c4c3c2)c(-n2c3ccccc3c3ccccc32)c1 ; N#Cc1ccc2oc3ccc4oc5ccc(-c6cccc(C#N)c6-n6c7ccccc7c7ccccc76)cc5c4c3c2c1 ; N#Cc1ccccc1-c1ccc2oc3ccc4oc5ccc(-c6cccc(C#N)c6-n6c7ccccc7c7ccccc76)cc5c4c3c2c1]

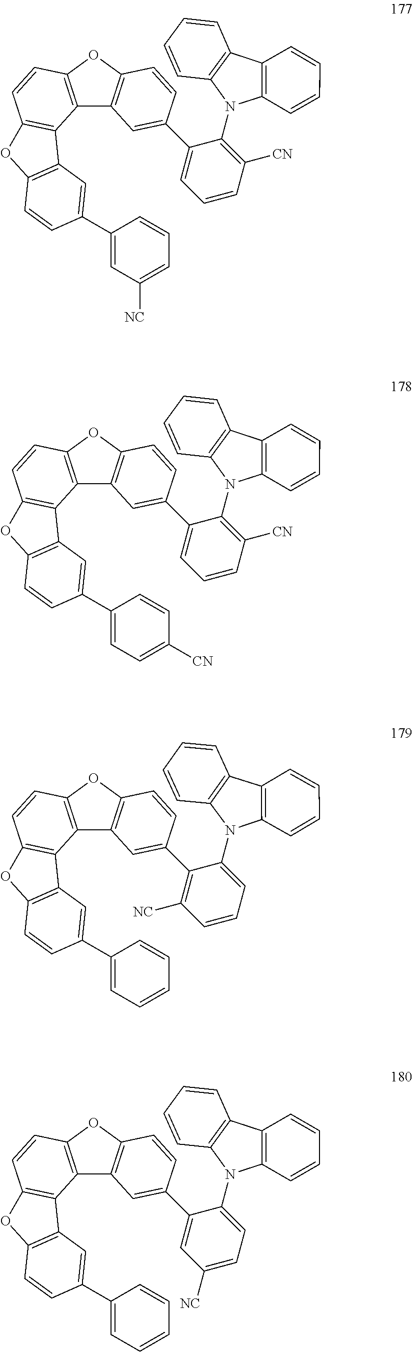 [SMILES: N#Cc1cccc(-c2ccc3oc4ccc5oc6ccc(-c7cccc(C#N)c7-n7c8ccccc8c8ccccc87)cc6c5c4c3c2)c1 ; N#Cc1ccc(-c2ccc3oc4ccc5oc6ccc(-c7cccc(C#N)c7-n7c8ccccc8c8ccccc87)cc6c5c4c3c2)cc1 ; N#Cc1cccc(-n2c3ccccc3c3ccccc32)c1-c1ccc2oc3ccc4oc5ccc(-c6ccccc6)cc5c4c3c2c1 ; N#Cc1ccc(-n2c3ccccc3c3ccccc32)c(-c2ccc3oc4ccc5oc6ccc(-c7ccccc7)cc6c5c4c3c2)c1]

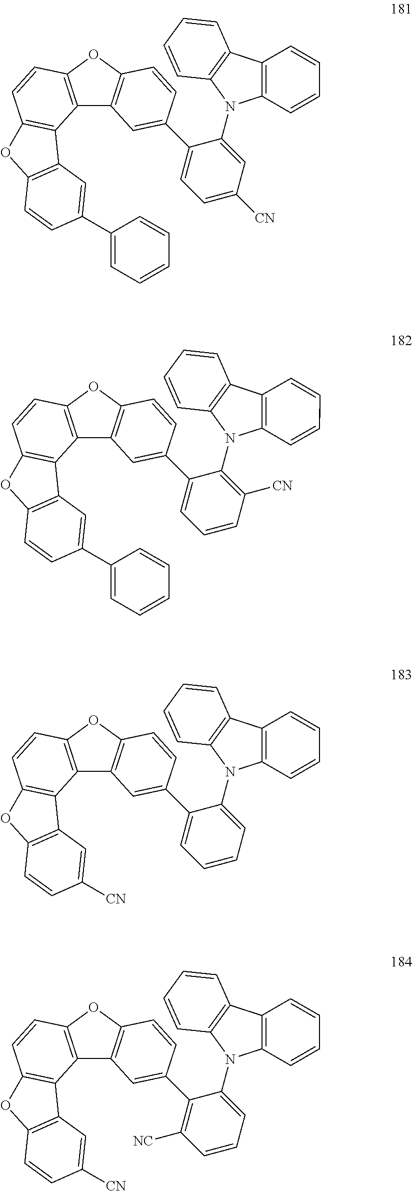 [SMILES: N#Cc1ccc(-c2ccc3oc4ccc5oc6ccc(-c7ccccc7)cc6c5c4c3c2)c(-n2c3ccccc3c3ccccc32)c1 ; N#Cc1cccc(-c2ccc3oc4ccc5oc6ccc(-c7ccccc7)cc6c5c4c3c2)c1-n1c2ccccc2c2ccccc21 ; N#Cc1ccc2oc3ccc4oc5ccc(-c6ccccc6-n6c7ccccc7c7ccccc76)cc5c4c3c2c1 ; N#Cc1ccc2oc3ccc4oc5ccc(-c6c(C#N)cccc6-n6c7ccccc7c7ccccc76)cc5c4c3c2c1]

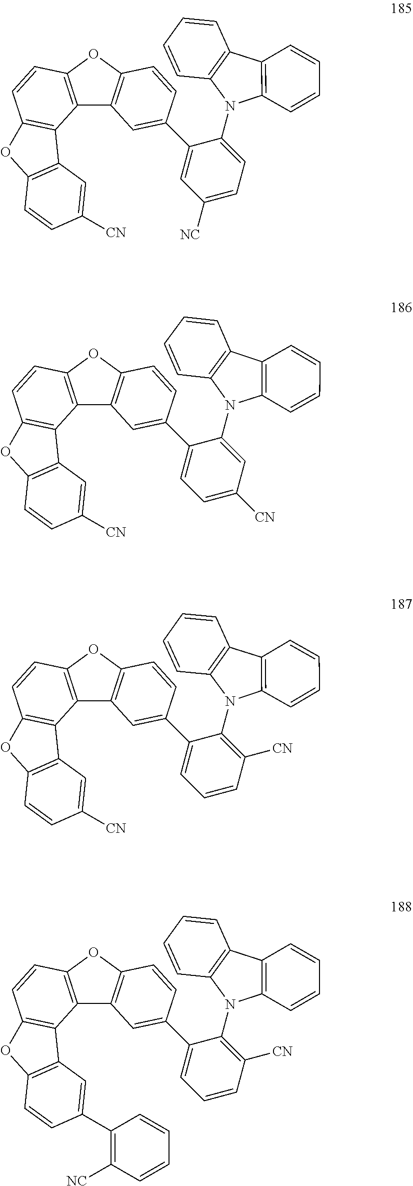 [SMILES: N#Cc1ccc(-n2c3ccccc3c3ccccc32)c(-c2ccc3oc4ccc5oc6ccc(C#N)cc6c5c4c3c2)c1 ; N#Cc1ccc(-c2ccc3oc4ccc5oc6ccc(C#N)cc6c5c4c3c2)c(-n2c3ccccc3c3ccccc32)c1 ; N#Cc1ccc2oc3ccc4oc5ccc(-c6cccc(C#N)c6-n6c7ccccc7c7ccccc76)cc5c4c3c2c1 ; N#Cc1ccccc1-c1ccc2oc3ccc4oc5ccc(-c6cccc(C#N)c6-n6c7ccccc7c7ccccc76)cc5c4c3c2c1]

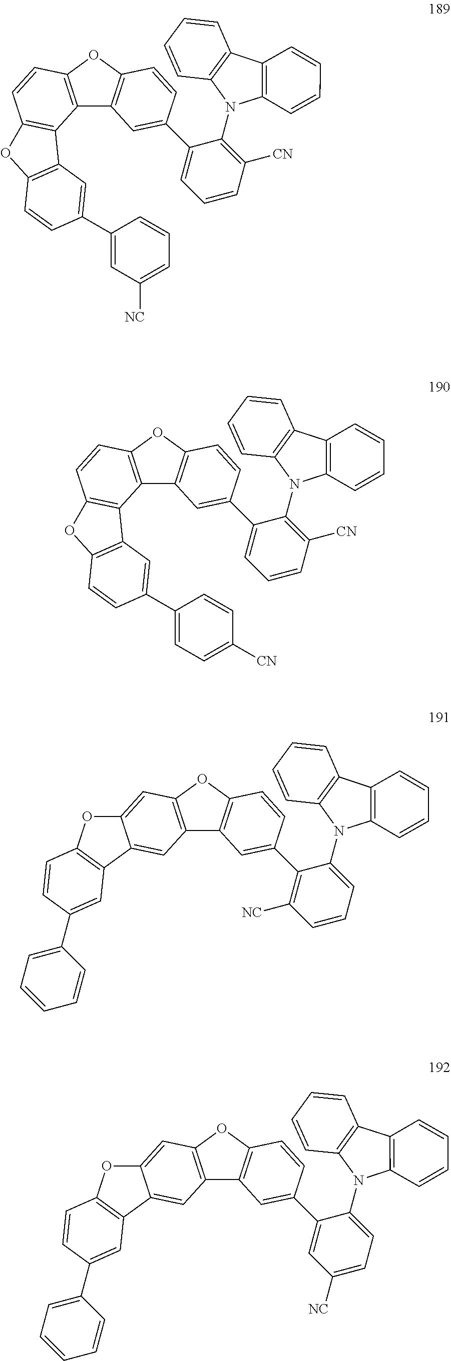 [SMILES: N#Cc1cccc(-c2ccc3oc4ccc5oc6ccc(-c7cccc(C#N)c7-n7c8ccccc8c8ccccc87)cc6c5c4c3c2)c1 ; N#Cc1ccc(-c2ccc3oc4ccc5oc6ccc(-c7cccc(C#N)c7-n7c8ccccc8c8ccccc87)cc6c5c4c3c2)cc1 ; N#Cc1cccc(-n2c3ccccc3c3ccccc32)c1-c1ccc2oc3cc4oc5ccc(-c6ccccc6)cc5c4cc3c2c1 ; N#Cc1ccc(-n2c3ccccc3c3ccccc32)c(-c2ccc3oc4cc5oc6ccc(-c7ccccc7)cc6c5cc4c3c2)c1]

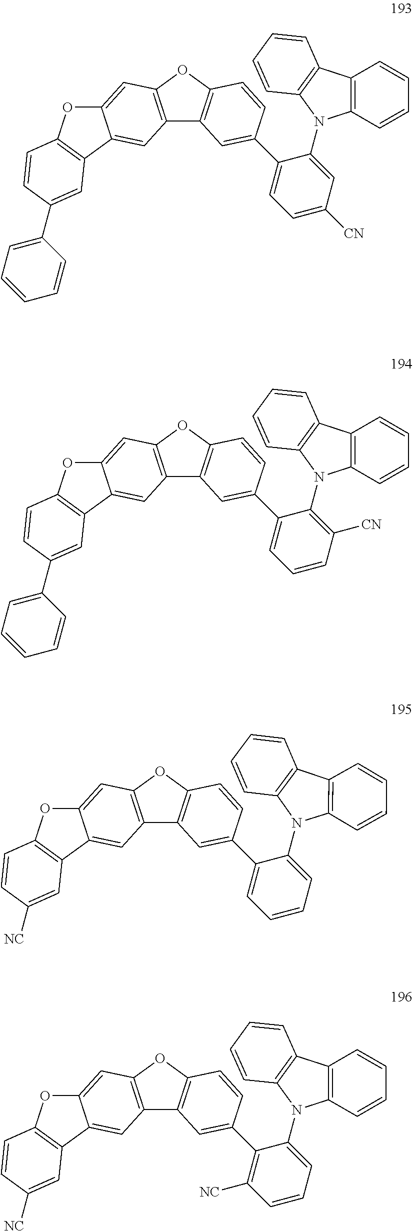 [SMILES: N#Cc1ccc(-c2ccc3oc4cc5oc6ccc(-c7ccccc7)cc6c5cc4c3c2)c(-n2c3ccccc3c3ccccc32)c1 ; N#Cc1cccc(-c2ccc3oc4cc5oc6ccc(-c7ccccc7)cc6c5cc4c3c2)c1-n1c2ccccc2c2ccccc21 ; N#Cc1ccc2oc3cc4oc5ccc(-c6ccccc6-n6c7ccccc7c7ccccc76)cc5c4cc3c2c1 ; N#Cc1ccc2oc3cc4oc5ccc(-c6c(C#N)cccc6-n6c7ccccc7c7ccccc76)cc5c4cc3c2c1]

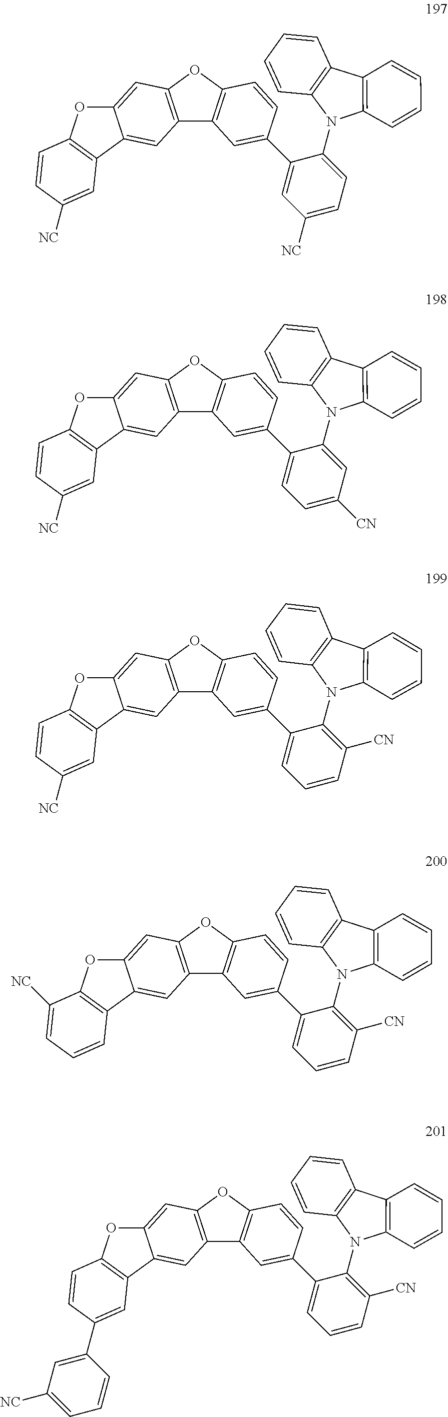 [SMILES: N#Cc1ccc(-n2c3ccccc3c3ccccc32)c(-c2ccc3oc4cc5oc6ccc(C#N)cc6c5cc4c3c2)c1 ; N#Cc1ccc(-c2ccc3oc4cc5oc6ccc(C#N)cc6c5cc4c3c2)c(-n2c3ccccc3c3ccccc32)c1 ; N#Cc1ccc2oc3cc4oc5ccc(-c6cccc(C#N)c6-n6c7ccccc7c7ccccc76)cc5c4cc3c2c1 ; N#Cc1cccc(-c2ccc3oc4cc5oc6c(C#N)cccc6c5cc4c3c2)c1-n1c2ccccc2c2ccccc21 ; N#Cc1cccc(-c2ccc3oc4cc5oc6ccc(-c7cccc(C#N)c7-n7c8ccccc8c8ccccc87)cc6c5cc4c3c2)c1]

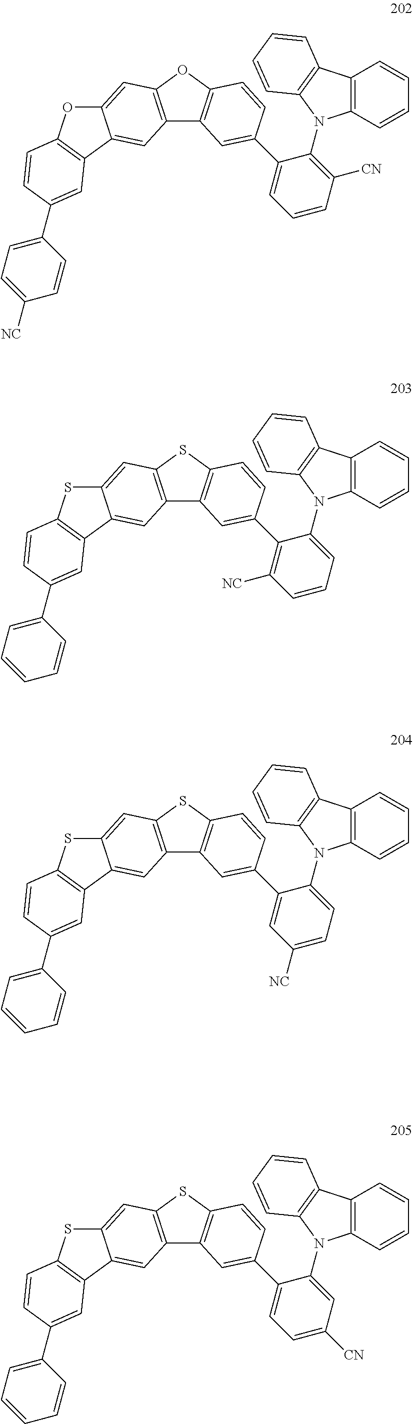 [SMILES: N#Cc1ccc(-c2ccc3oc4cc5oc6ccc(-c7cccc(C#N)c7-n7c8ccccc8c8ccccc87)cc6c5cc4c3c2)cc1 ; N#Cc1cccc(-n2c3ccccc3c3ccccc32)c1-c1ccc2sc3cc4sc5ccc(-c6ccccc6)cc5c4cc3c2c1 ; N#Cc1ccc(-n2c3ccccc3c3ccccc32)c(-c2ccc3sc4cc5sc6ccc(-c7ccccc7)cc6c5cc4c3c2)c1 ; N#Cc1ccc(-c2ccc3sc4cc5sc6ccc(-c7ccccc7)cc6c5cc4c3c2)c(-n2c3ccccc3c3ccccc32)c1]

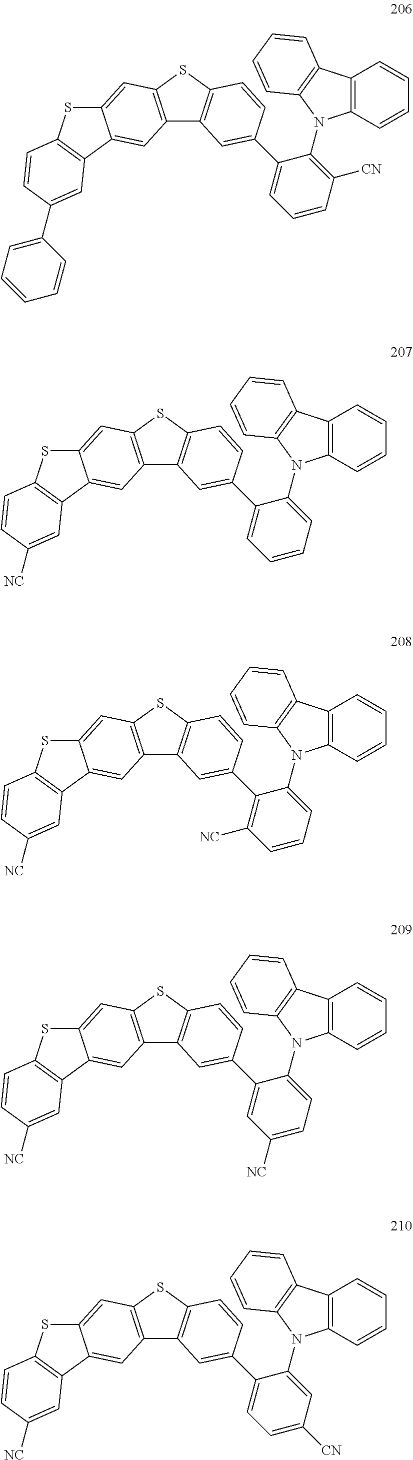 [SMILES: N#Cc1cccc(-c2ccc3sc4cc5sc6ccc(-c7ccccc7)cc6c5cc4c3c2)c1-n1c2ccccc2c2ccccc21 ; N#Cc1ccc2sc3cc4sc5ccc(-c6ccccc6-n6c7ccccc7c7ccccc76)cc5c4cc3c2c1 ; N#Cc1ccc2sc3cc4sc5ccc(-c6c(C#N)cccc6-n6c7ccccc7c7ccccc76)cc5c4cc3c2c1 ; N#Cc1ccc(-n2c3ccccc3c3ccccc32)c(-c2ccc3sc4cc5sc6ccc(C#N)cc6c5cc4c3c2)c1 ; N#Cc1ccc(-c2ccc3sc4cc5sc6ccc(C#N)cc6c5cc4c3c2)c(-n2c3ccccc3c3ccccc32)c1]

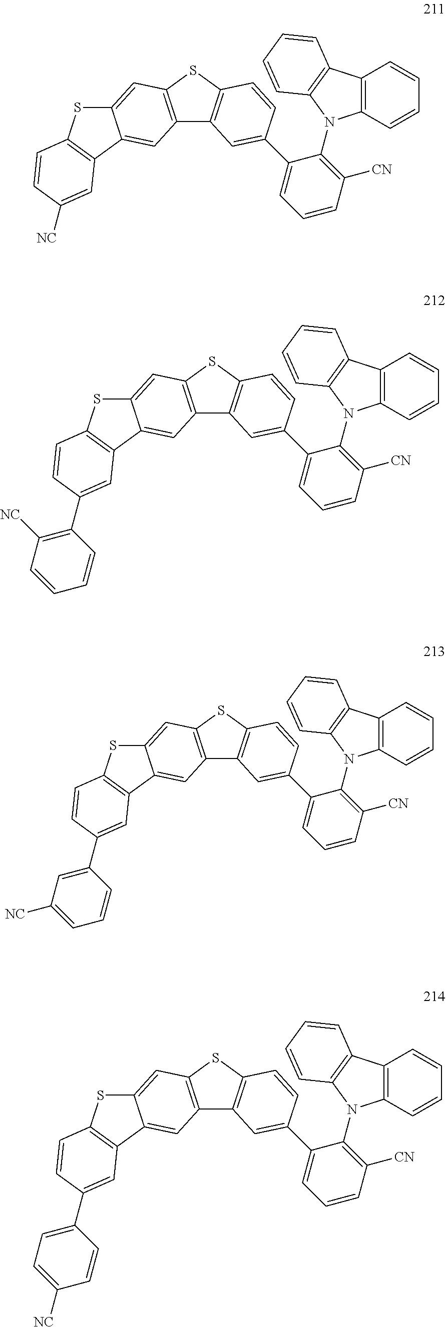 [SMILES: N#Cc1ccc2sc3cc4sc5ccc(-c6cccc(C#N)c6-n6c7ccccc7c7ccccc76)cc5c4cc3c2c1 ; N#Cc1ccccc1-c1ccc2sc3cc4sc5ccc(-c6cccc(C#N)c6-n6c7ccccc7c7ccccc76)cc5c4cc3c2c1 ; N#Cc1cccc(-c2ccc3sc4cc5sc6ccc(-c7cccc(C#N)c7-n7c8ccccc8c8ccccc87)cc6c5cc4c3c2)c1 ; N#Cc1ccc(-c2ccc3sc4cc5sc6ccc(-c7cccc(C#N)c7-n7c8ccccc8c8ccccc87)cc6c5cc4c3c2)cc1]

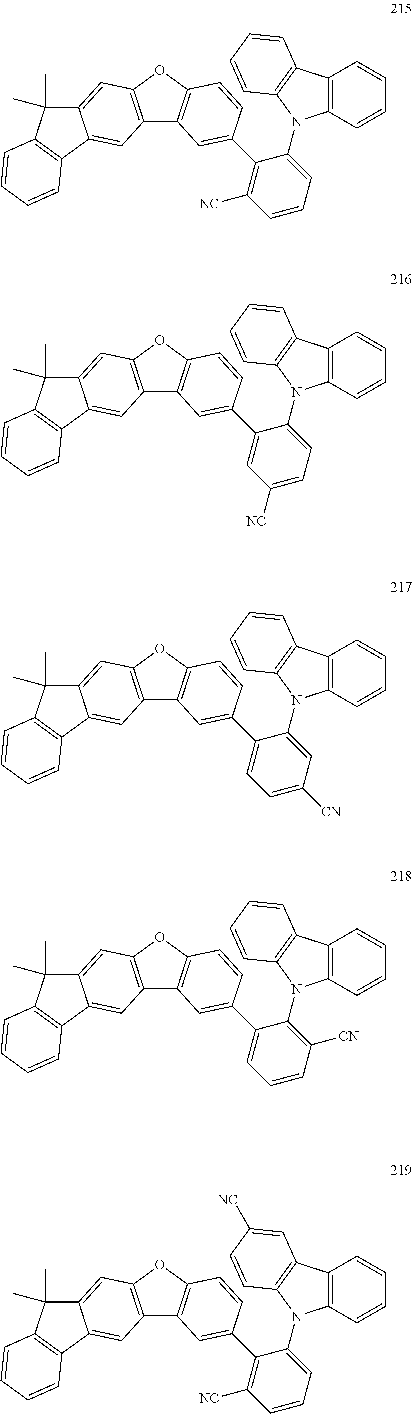 [SMILES: CC1(C)c2ccccc2-c2cc3c(cc21)oc1ccc(-c2c(C#N)cccc2-n2c4ccccc4c4ccccc42)cc13 ; CC1(C)c2ccccc2-c2cc3c(cc21)oc1ccc(-c2cc(C#N)ccc2-n2c4ccccc4c4ccccc42)cc13 ; CC1(C)c2ccccc2-c2cc3c(cc21)oc1ccc(-c2ccc(C#N)cc2-n2c4ccccc4c4ccccc42)cc13 ; CC1(C)c2ccccc2-c2cc3c(cc21)oc1ccc(-c2cccc(C#N)c2-n2c4ccccc4c4ccccc42)cc13 ; CC1(C)c2ccccc2-c2cc3c(cc21)oc1ccc(-c2c(C#N)cccc2-n2c4ccccc4c4cc(C#N)ccc42)cc13]

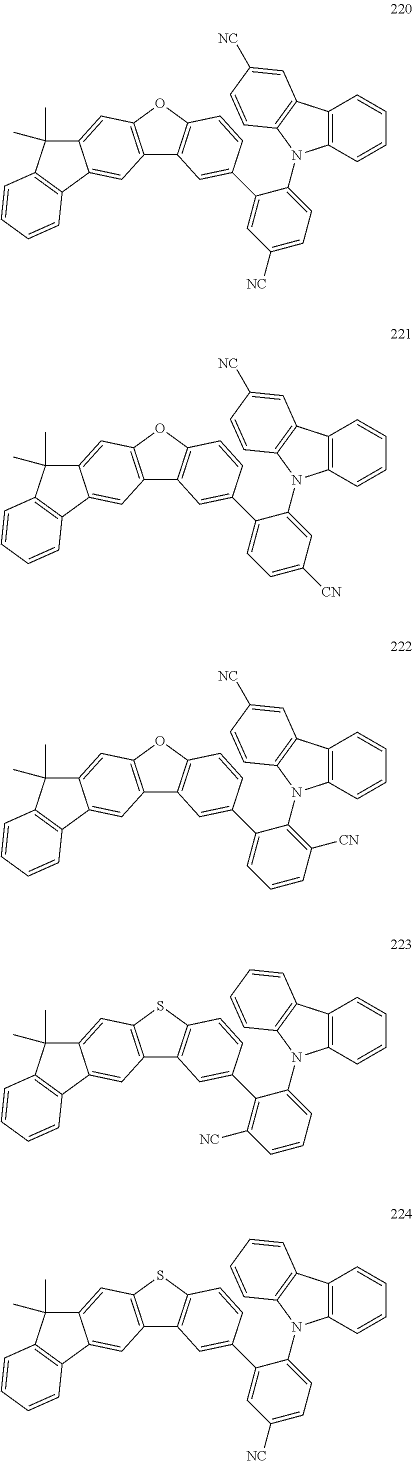 [SMILES: CC1(C)c2ccccc2-c2cc3c(cc21)oc1ccc(-c2cc(C#N)ccc2-n2c4ccccc4c4cc(C#N)ccc42)cc13 ; CC1(C)c2ccccc2-c2cc3c(cc21)oc1ccc(-c2ccc(C#N)cc2-n2c4ccccc4c4cc(C#N)ccc42)cc13 ; CC1(C)c2ccccc2-c2cc3c(cc21)oc1ccc(-c2cccc(C#N)c2-n2c4ccccc4c4cc(C#N)ccc42)cc13 ; CC1(C)c2ccccc2-c2cc3c(cc21)sc1ccc(-c2c(C#N)cccc2-n2c4ccccc4c4ccccc42)cc13 ; CC1(C)c2ccccc2-c2cc3c(cc21)sc1ccc(-c2cc(C#N)ccc2-n2c4ccccc4c4ccccc42)cc13]

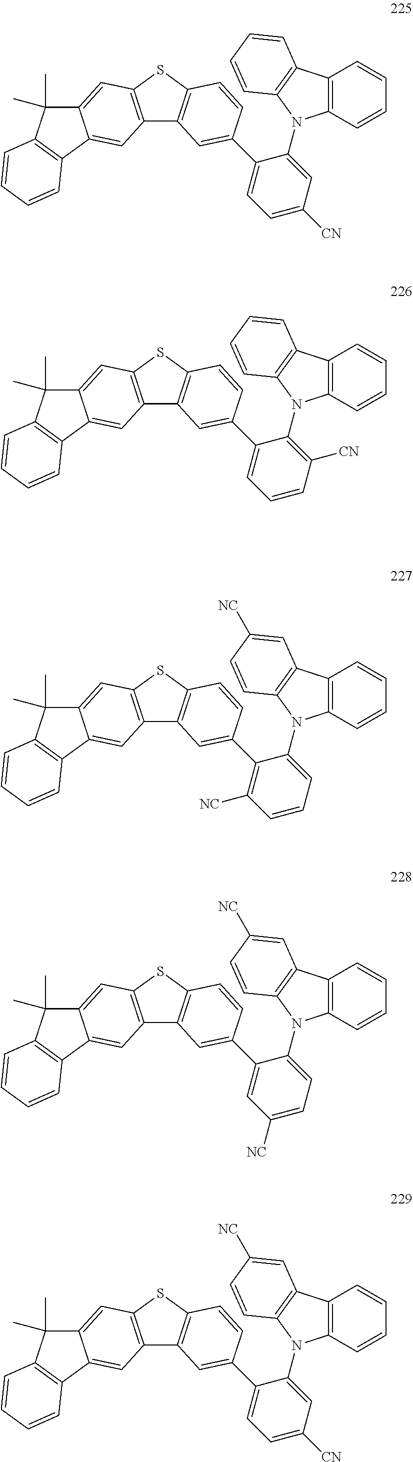 [SMILES: CC1(C)c2ccccc2-c2cc3c(cc21)sc1ccc(-c2ccc(C#N)cc2-n2c4ccccc4c4ccccc42)cc13 ; CC1(C)c2ccccc2-c2cc3c(cc21)sc1ccc(-c2cccc(C#N)c2-n2c4ccccc4c4ccccc42)cc13 ; CC1(C)c2ccccc2-c2cc3c(cc21)sc1ccc(-c2c(C#N)cccc2-n2c4ccccc4c4cc(C#N)ccc42)cc13 ; CC1(C)c2ccccc2-c2cc3c(cc21)sc1ccc(-c2cc(C#N)ccc2-n2c4ccccc4c4cc(C#N)ccc42)cc13 ; CC1(C)c2ccccc2-c2cc3c(cc21)sc1ccc(-c2ccc(C#N)cc2-n2c4ccccc4c4cc(C#N)ccc42)cc13]

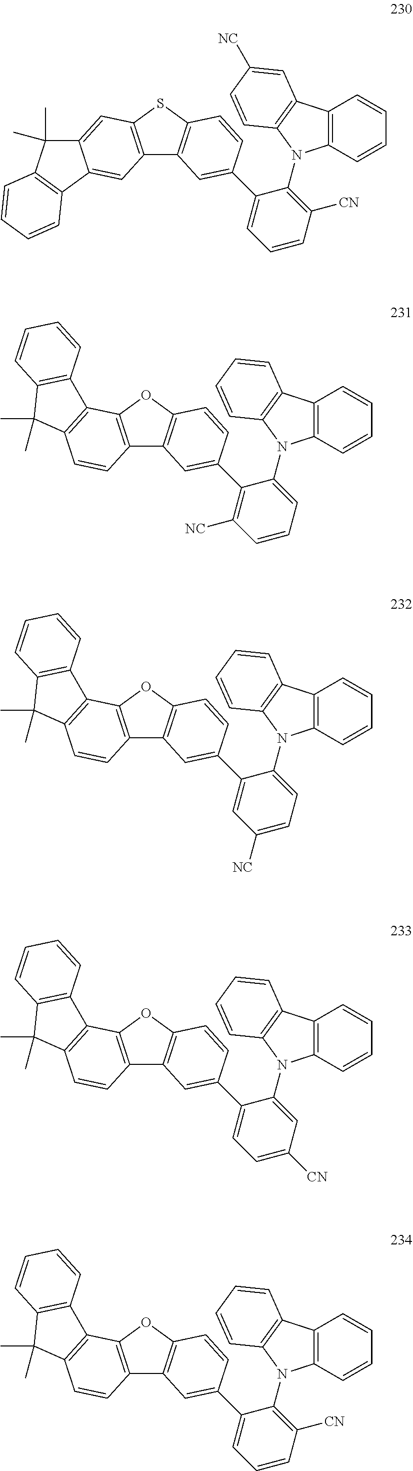 [SMILES: CC1(C)c2ccccc2-c2cc3c(cc21)sc1ccc(-c2cccc(C#N)c2-n2c4ccccc4c4cc(C#N)ccc42)cc13 ; CC1(C)c2ccccc2-c2c1ccc1c2oc2ccc(-c3c(C#N)cccc3-n3c4ccccc4c4ccccc43)cc21 ; CC1(C)c2ccccc2-c2c1ccc1c2oc2ccc(-c3cc(C#N)ccc3-n3c4ccccc4c4ccccc43)cc21 ; CC1(C)c2ccccc2-c2c1ccc1c2oc2ccc(-c3ccc(C#N)cc3-n3c4ccccc4c4ccccc43)cc21 ; CC1(C)c2ccccc2-c2c1ccc1c2oc2ccc(-c3cccc(C#N)c3-n3c4ccccc4c4ccccc43)cc21]

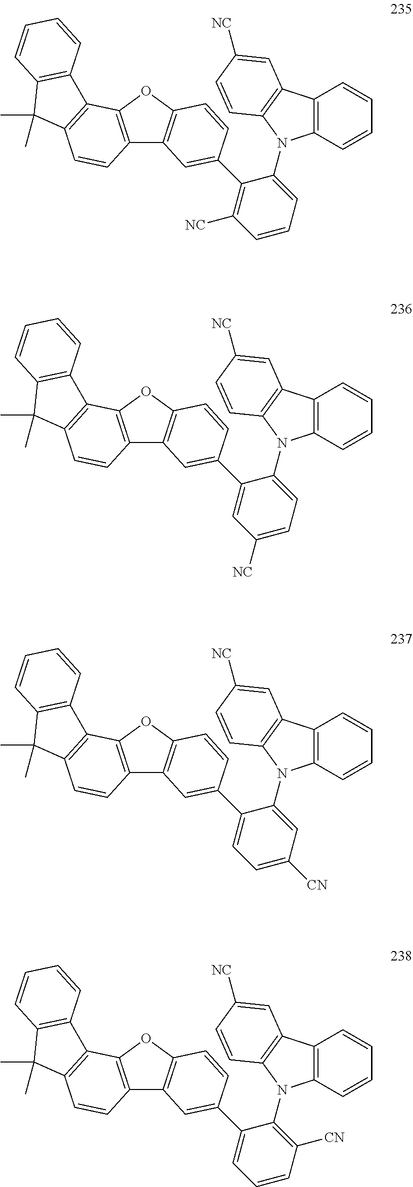 [SMILES: CC1(C)c2ccccc2-c2c1ccc1c2oc2ccc(-c3c(C#N)cccc3-n3c4ccccc4c4cc(C#N)ccc43)cc21 ; CC1(C)c2ccccc2-c2c1ccc1c2oc2ccc(-c3cc(C#N)ccc3-n3c4ccccc4c4cc(C#N)ccc43)cc21 ; CC1(C)c2ccccc2-c2c1ccc1c2oc2ccc(-c3ccc(C#N)cc3-n3c4ccccc4c4cc(C#N)ccc43)cc21 ; CC1(C)c2ccccc2-c2c1ccc1c2oc2ccc(-c3cccc(C#N)c3-n3c4ccccc4c4cc(C#N)ccc43)cc21]

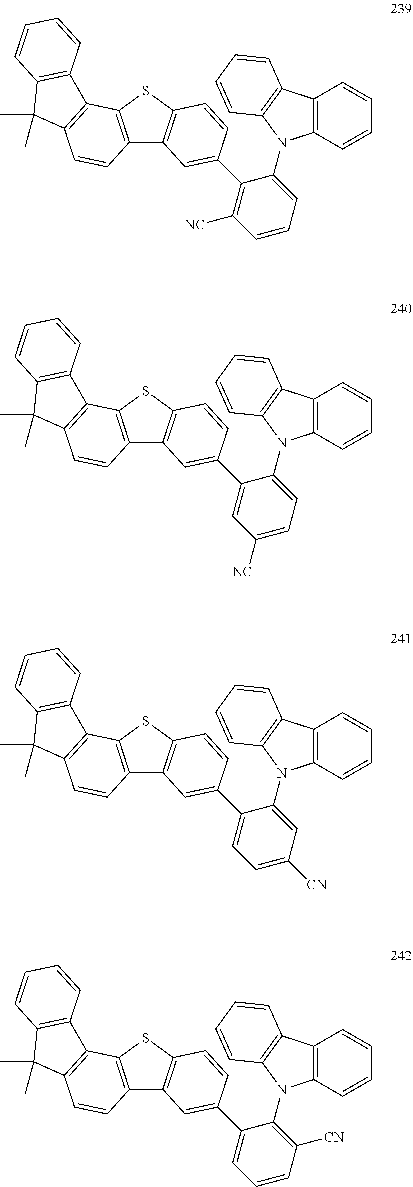 [SMILES: CC1(C)c2ccccc2-c2c1ccc1c2sc2ccc(-c3c(C#N)cccc3-n3c4ccccc4c4ccccc43)cc21 ; CC1(C)c2ccccc2-c2c1ccc1c2sc2ccc(-c3cc(C#N)ccc3-n3c4ccccc4c4ccccc43)cc21 ; CC1(C)c2ccccc2-c2c1ccc1c2sc2ccc(-c3ccc(C#N)cc3-n3c4ccccc4c4ccccc43)cc21 ; CC1(C)c2ccccc2-c2c1ccc1c2sc2ccc(-c3cccc(C#N)c3-n3c4ccccc4c4ccccc43)cc21]

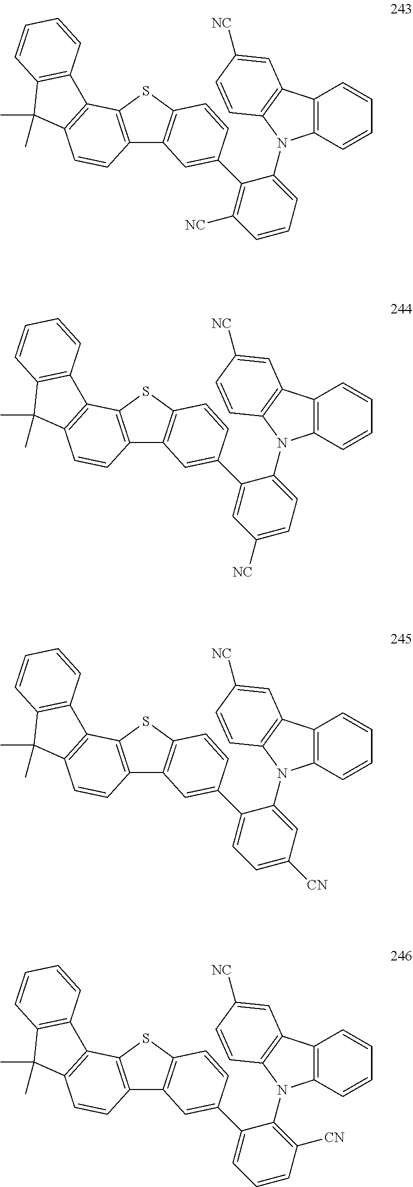 [SMILES: CC1(C)c2ccccc2-c2c1ccc1c2sc2ccc(-c3c(C#N)cccc3-n3c4ccccc4c4cc(C#N)ccc43)cc21 ; CC1(C)c2ccccc2-c2c1ccc1c2sc2ccc(-c3cc(C#N)ccc3-n3c4ccccc4c4cc(C#N)ccc43)cc21 ; CC1(C)c2ccccc2-c2c1ccc1c2sc2ccc(-c3ccc(C#N)cc3-n3c4ccccc4c4cc(C#N)ccc43)cc21 ; CC1(C)c2ccccc2-c2c1ccc1c2sc2ccc(-c3cccc(C#N)c3-n3c4ccccc4c4cc(C#N)ccc43)cc21]

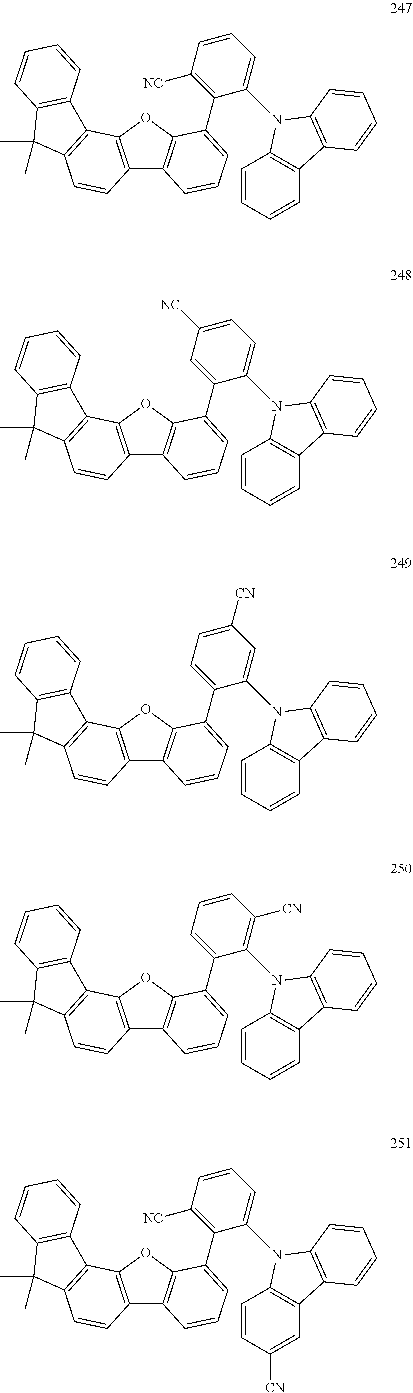 [SMILES: CC1(C)c2ccccc2-c2c1ccc1c2oc2c(-c3c(C#N)cccc3-n3c4ccccc4c4ccccc43)cccc21 ; CC1(C)c2ccccc2-c2c1ccc1c2oc2c(-c3cc(C#N)ccc3-n3c4ccccc4c4ccccc43)cccc21 ; CC1(C)c2ccccc2-c2c1ccc1c2oc2c(-c3ccc(C#N)cc3-n3c4ccccc4c4ccccc43)cccc21 ; CC1(C)c2ccccc2-c2c1ccc1c2oc2c(-c3cccc(C#N)c3-n3c4ccccc4c4ccccc43)cccc21 ; CC1(C)c2ccccc2-c2c1ccc1c2oc2c(-c3c(C#N)cccc3-n3c4ccccc4c4cc(C#N)ccc43)cccc21]

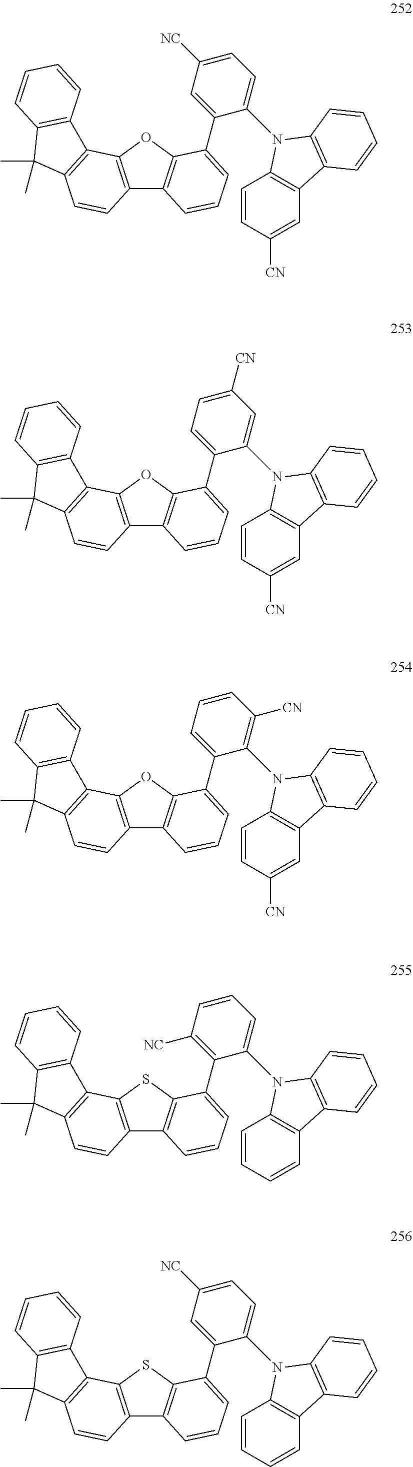 [SMILES: CC1(C)c2ccccc2-c2c1ccc1c2oc2c(-c3cc(C#N)ccc3-n3c4ccccc4c4cc(C#N)ccc43)cccc21 ; CC1(C)c2ccccc2-c2c1ccc1c2oc2c(-c3ccc(C#N)cc3-n3c4ccccc4c4cc(C#N)ccc43)cccc21 ; CC1(C)c2ccccc2-c2c1ccc1c2oc2c(-c3cccc(C#N)c3-n3c4ccccc4c4cc(C#N)ccc43)cccc21 ; CC1(C)c2ccccc2-c2c1ccc1c2sc2c(-c3c(C#N)cccc3-n3c4ccccc4c4ccccc43)cccc21 ; CC1(C)c2ccccc2-c2c1ccc1c2sc2c(-c3cc(C#N)ccc3-n3c4ccccc4c4ccccc43)cccc21]

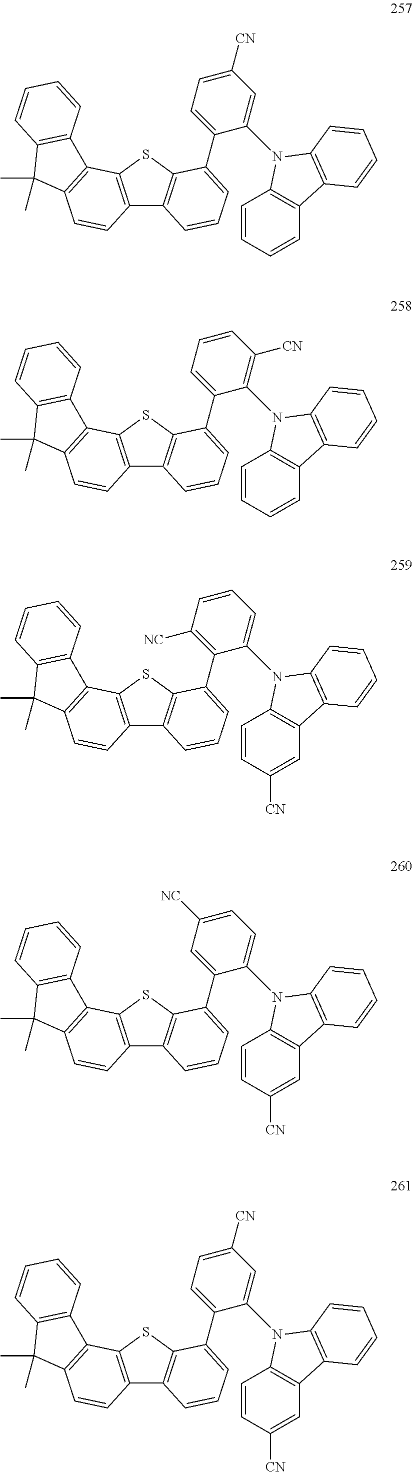 [SMILES: CC1(C)c2ccccc2-c2c1ccc1c2sc2c(-c3ccc(C#N)cc3-n3c4ccccc4c4ccccc43)cccc21 ; CC1(C)c2ccccc2-c2c1ccc1c2sc2c(-c3cccc(C#N)c3-n3c4ccccc4c4ccccc43)cccc21 ; CC1(C)c2ccccc2-c2c1ccc1c2sc2c(-c3c(C#N)cccc3-n3c4ccccc4c4cc(C#N)ccc43)cccc21 ; CC1(C)c2ccccc2-c2c1ccc1c2sc2c(-c3cc(C#N)ccc3-n3c4ccccc4c4cc(C#N)ccc43)cccc21 ; CC1(C)c2ccccc2-c2c1ccc1c2sc2c(-c3ccc(C#N)cc3-n3c4ccccc4c4cc(C#N)ccc43)cccc21]

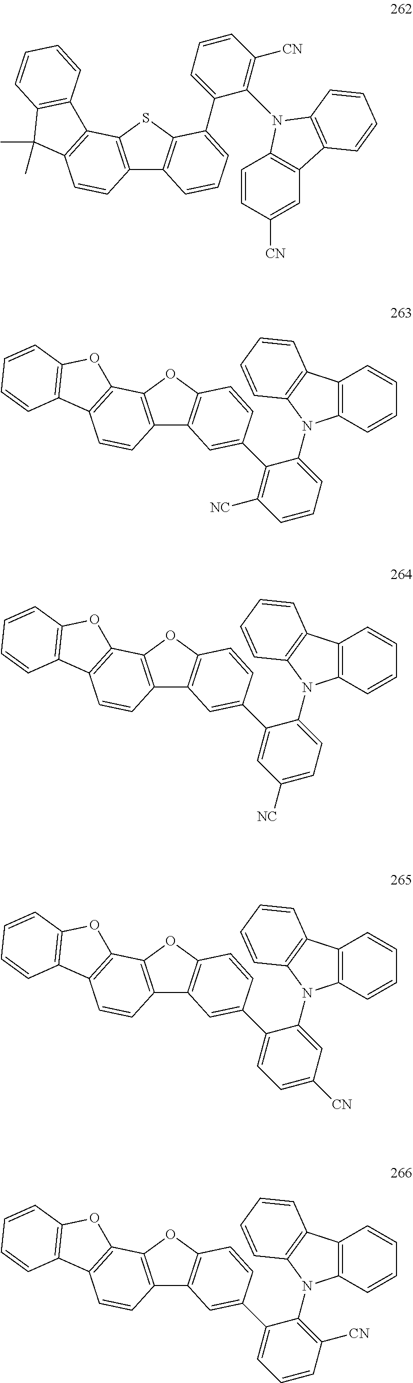 [SMILES: CC1(C)c2ccccc2-c2c1ccc1c2sc2c(-c3cccc(C#N)c3-n3c4ccccc4c4cc(C#N)ccc43)cccc21 ; N#Cc1cccc(-n2c3ccccc3c3ccccc32)c1-c1ccc2oc3c(ccc4c5ccccc5oc43)c2c1 ; N#Cc1ccc(-n2c3ccccc3c3ccccc32)c(-c2ccc3oc4c(ccc5c6ccccc6oc54)c3c2)c1 ; N#Cc1ccc(-c2ccc3oc4c(ccc5c6ccccc6oc54)c3c2)c(-n2c3ccccc3c3ccccc32)c1 ; N#Cc1cccc(-c2ccc3oc4c(ccc5c6ccccc6oc54)c3c2)c1-n1c2ccccc2c2ccccc21]

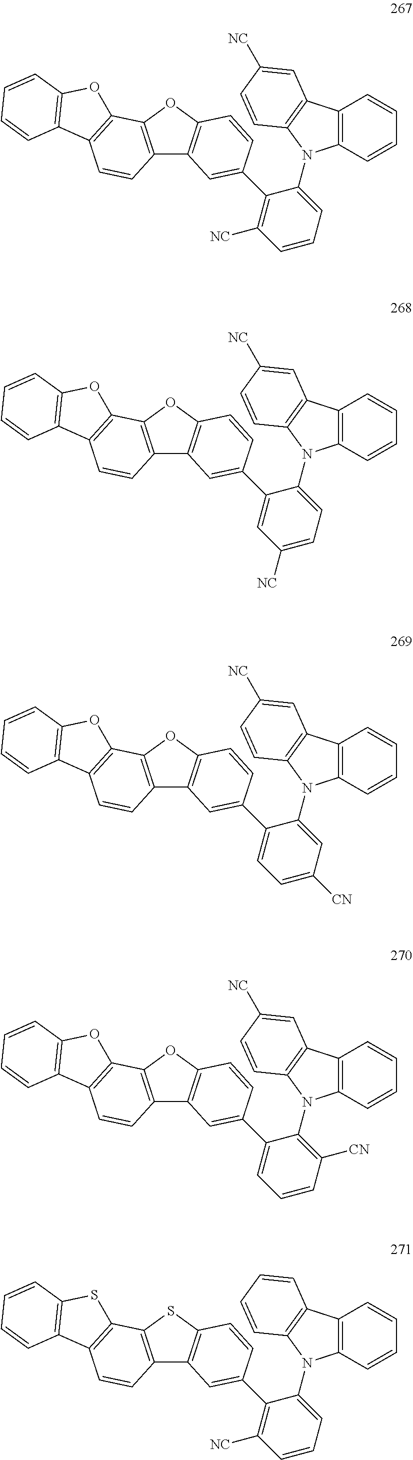 [SMILES: N#Cc1ccc2c(c1)c1ccccc1n2-c1cccc(C#N)c1-c1ccc2oc3c(ccc4c5ccccc5oc43)c2c1 ; N#Cc1ccc(-n2c3ccccc3c3cc(C#N)ccc32)c(-c2ccc3oc4c(ccc5c6ccccc6oc54)c3c2)c1 ; N#Cc1ccc(-c2ccc3oc4c(ccc5c6ccccc6oc54)c3c2)c(-n2c3ccccc3c3cc(C#N)ccc32)c1 ; N#Cc1ccc2c(c1)c1ccccc1n2-c1c(C#N)cccc1-c1ccc2oc3c(ccc4c5ccccc5oc43)c2c1 ; N#Cc1cccc(-n2c3ccccc3c3ccccc32)c1-c1ccc2sc3c(ccc4c5ccccc5sc43)c2c1]

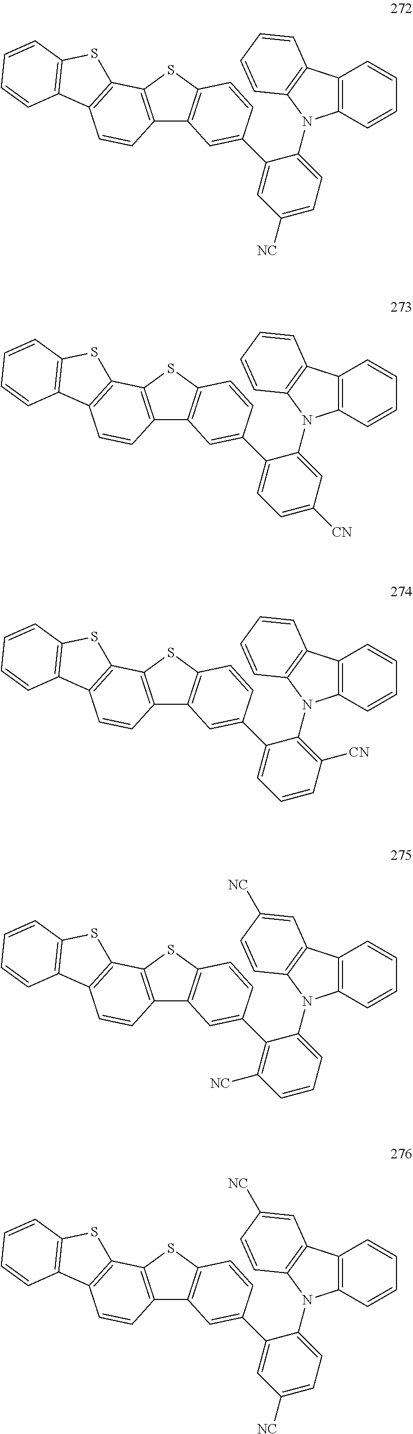 [SMILES: N#Cc1ccc(-n2c3ccccc3c3ccccc32)c(-c2ccc3sc4c(ccc5c6ccccc6sc54)c3c2)c1 ; N#Cc1ccc(-c2ccc3sc4c(ccc5c6ccccc6sc54)c3c2)c(-n2c3ccccc3c3ccccc32)c1 ; N#Cc1cccc(-c2ccc3sc4c(ccc5c6ccccc6sc54)c3c2)c1-n1c2ccccc2c2ccccc21 ; N#Cc1ccc2c(c1)c1ccccc1n2-c1cccc(C#N)c1-c1ccc2sc3c(ccc4c5ccccc5sc43)c2c1 ; N#Cc1ccc(-n2c3ccccc3c3cc(C#N)ccc32)c(-c2ccc3sc4c(ccc5c6ccccc6sc54)c3c2)c1]

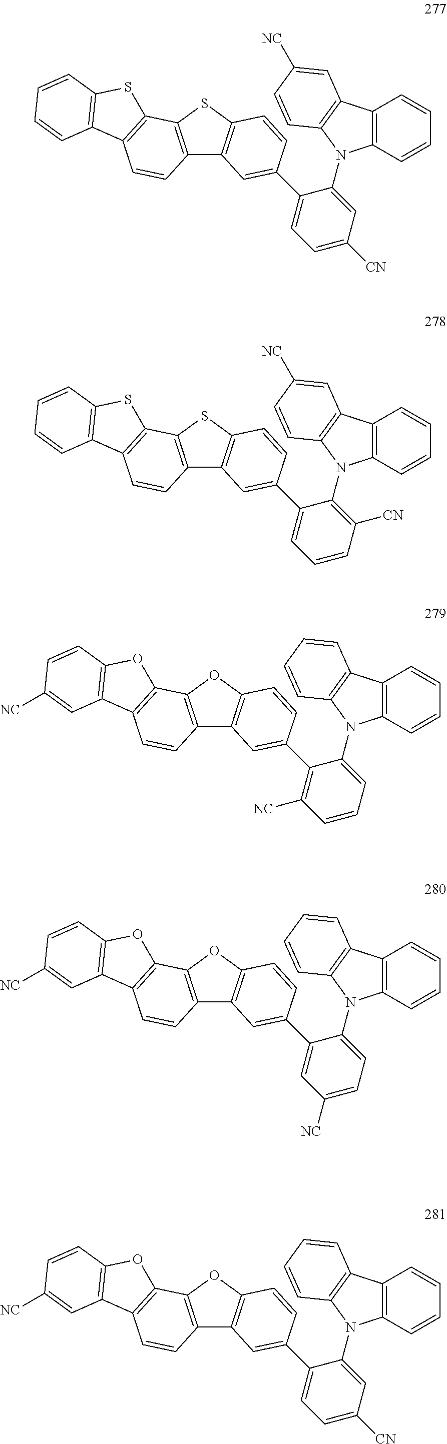 [SMILES: N#Cc1ccc(-c2ccc3sc4c(ccc5c6ccccc6sc54)c3c2)c(-n2c3ccccc3c3cc(C#N)ccc32)c1 ; N#Cc1ccc2c(c1)c1ccccc1n2-c1c(C#N)cccc1-c1ccc2sc3c(ccc4c5ccccc5sc43)c2c1 ; N#Cc1ccc2oc3c(ccc4c5cc(-c6c(C#N)cccc6-n6c7ccccc7c7ccccc76)ccc5oc43)c2c1 ; N#Cc1ccc(-n2c3ccccc3c3ccccc32)c(-c2ccc3oc4c(ccc5c6cc(C#N)ccc6oc54)c3c2)c1 ; N#Cc1ccc(-c2ccc3oc4c(ccc5c6cc(C#N)ccc6oc54)c3c2)c(-n2c3ccccc3c3ccccc32)c1]

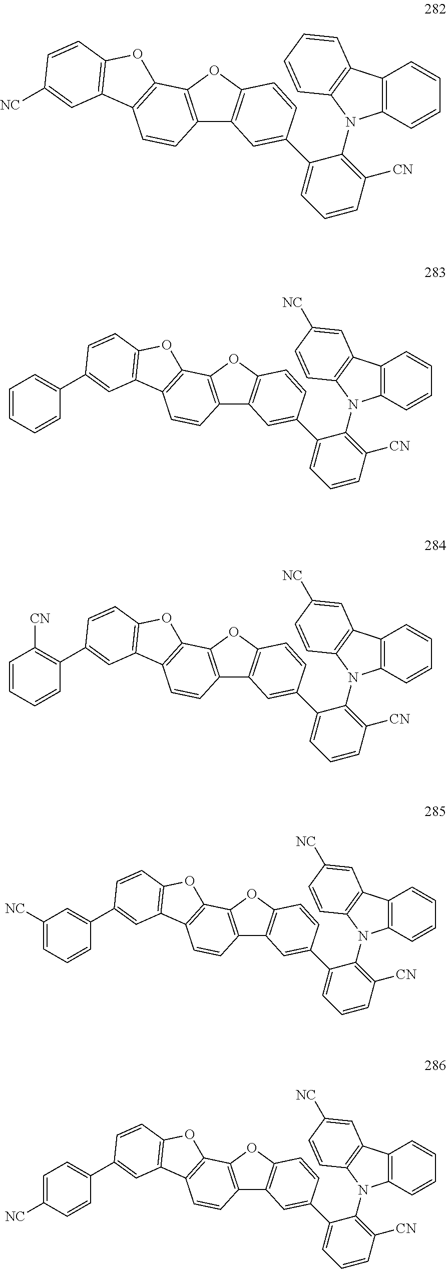 [SMILES: N#Cc1ccc2oc3c(ccc4c5cc(-c6cccc(C#N)c6-n6c7ccccc7c7ccccc76)ccc5oc43)c2c1 ; N#Cc1ccc2c(c1)c1ccccc1n2-c1c(C#N)cccc1-c1ccc2oc3c(ccc4c5cc(-c6ccccc6)ccc5oc43)c2c1 ; N#Cc1ccc2c(c1)c1ccccc1n2-c1c(C#N)cccc1-c1ccc2oc3c(ccc4c5cc(-c6ccccc6C#N)ccc5oc43)c2c1 ; N#Cc1cccc(-c2ccc3oc4c(ccc5c6cc(-c7cccc(C#N)c7-n7c8ccccc8c8cc(C#N)ccc87)ccc6oc54)c3c2)c1 ; N#Cc1ccc(-c2ccc3oc4c(ccc5c6cc(-c7cccc(C#N)c7-n7c8ccccc8c8cc(C#N)ccc87)ccc6oc54)c3c2)cc1]

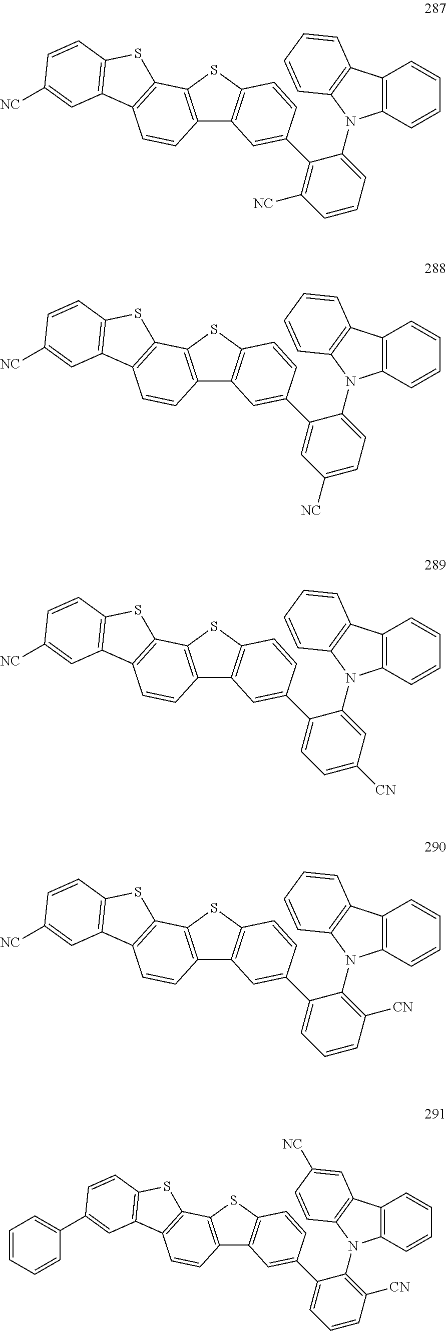 [SMILES: N#Cc1ccc2sc3c(ccc4c5cc(-c6c(C#N)cccc6-n6c7ccccc7c7ccccc76)ccc5sc43)c2c1 ; N#Cc1ccc(-n2c3ccccc3c3ccccc32)c(-c2ccc3sc4c(ccc5c6cc(C#N)ccc6sc54)c3c2)c1 ; N#Cc1ccc(-c2ccc3sc4c(ccc5c6cc(C#N)ccc6sc54)c3c2)c(-n2c3ccccc3c3ccccc32)c1 ; N#Cc1ccc2sc3c(ccc4c5cc(-c6cccc(C#N)c6-n6c7ccccc7c7ccccc76)ccc5sc43)c2c1 ; N#Cc1ccc2c(c1)c1ccccc1n2-c1c(C#N)cccc1-c1ccc2sc3c(ccc4c5cc(-c6ccccc6)ccc5sc43)c2c1]

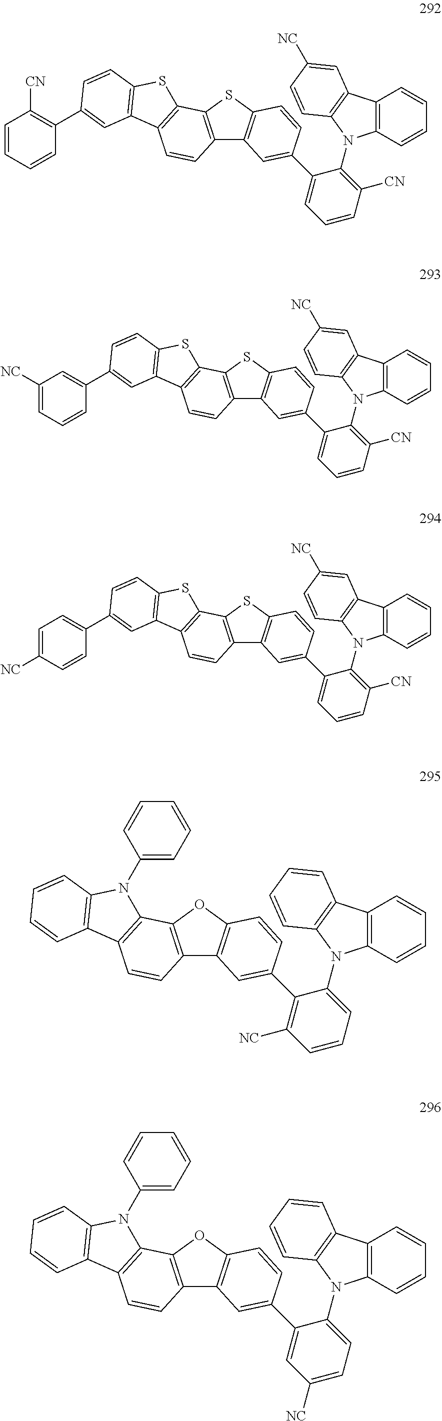 [SMILES: N#Cc1ccc2c(c1)c1ccccc1n2-c1c(C#N)cccc1-c1ccc2sc3c(ccc4c5cc(-c6ccccc6C#N)ccc5sc43)c2c1 ; N#Cc1cccc(-c2ccc3sc4c(ccc5c6cc(-c7cccc(C#N)c7-n7c8ccccc8c8cc(C#N)ccc87)ccc6sc54)c3c2)c1 ; N#Cc1ccc(-c2ccc3sc4c(ccc5c6cc(-c7cccc(C#N)c7-n7c8ccccc8c8cc(C#N)ccc87)ccc6sc54)c3c2)cc1 ; N#Cc1cccc(-n2c3ccccc3c3ccccc32)c1-c1ccc2oc3c(ccc4c5ccccc5n(-c5ccccc5)c43)c2c1 ; N#Cc1ccc(-n2c3ccccc3c3ccccc32)c(-c2ccc3oc4c(ccc5c6ccccc6n(-c6ccccc6)c54)c3c2)c1]

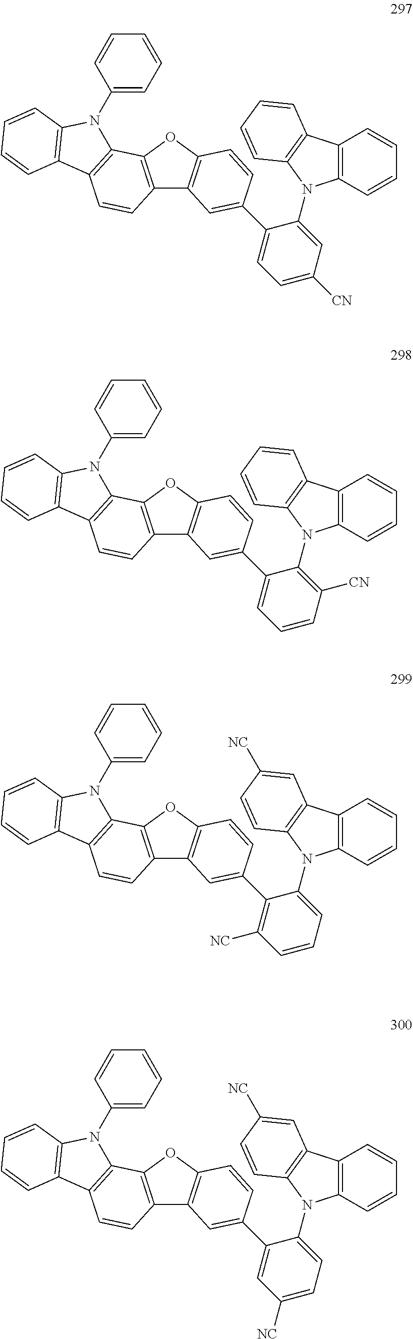 [SMILES: N#Cc1ccc(-c2ccc3oc4c(ccc5c6ccccc6n(-c6ccccc6)c54)c3c2)c(-n2c3ccccc3c3ccccc32)c1 ; N#Cc1cccc(-c2ccc3oc4c(ccc5c6ccccc6n(-c6ccccc6)c54)c3c2)c1-n1c2ccccc2c2ccccc21 ; N#Cc1ccc2c(c1)c1ccccc1n2-c1cccc(C#N)c1-c1ccc2oc3c(ccc4c5ccccc5n(-c5ccccc5)c43)c2c1 ; N#Cc1ccc(-n2c3ccccc3c3cc(C#N)ccc32)c(-c2ccc3oc4c(ccc5c6ccccc6n(-c6ccccc6)c54)c3c2)c1]

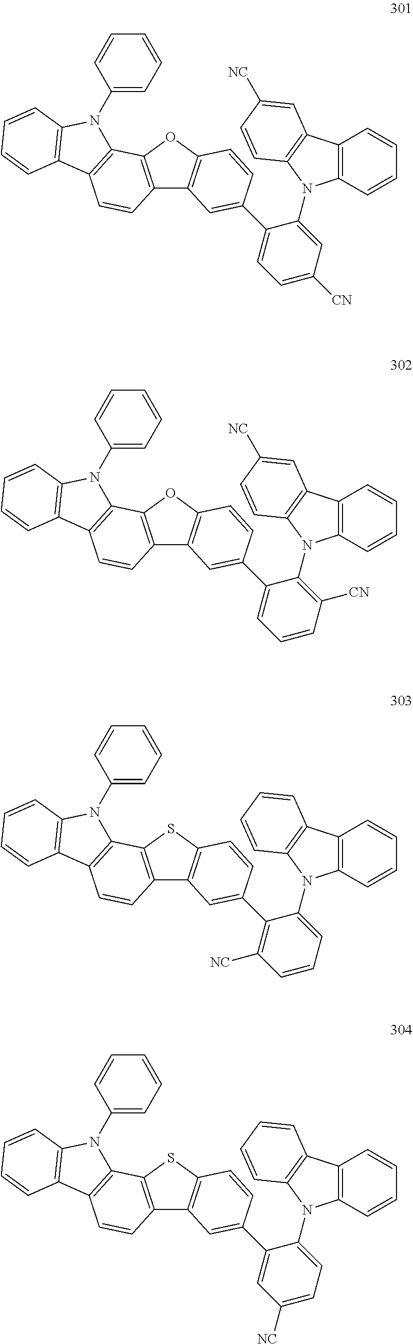 [SMILES: N#Cc1ccc(-c2ccc3oc4c(ccc5c6ccccc6n(-c6ccccc6)c54)c3c2)c(-n2c3ccccc3c3cc(C#N)ccc32)c1 ; N#Cc1ccc2c(c1)c1ccccc1n2-c1c(C#N)cccc1-c1ccc2oc3c(ccc4c5ccccc5n(-c5ccccc5)c43)c2c1 ; N#Cc1cccc(-n2c3ccccc3c3ccccc32)c1-c1ccc2sc3c(ccc4c5ccccc5n(-c5ccccc5)c43)c2c1 ; N#Cc1ccc(-n2c3ccccc3c3ccccc32)c(-c2ccc3sc4c(ccc5c6ccccc6n(-c6ccccc6)c54)c3c2)c1]

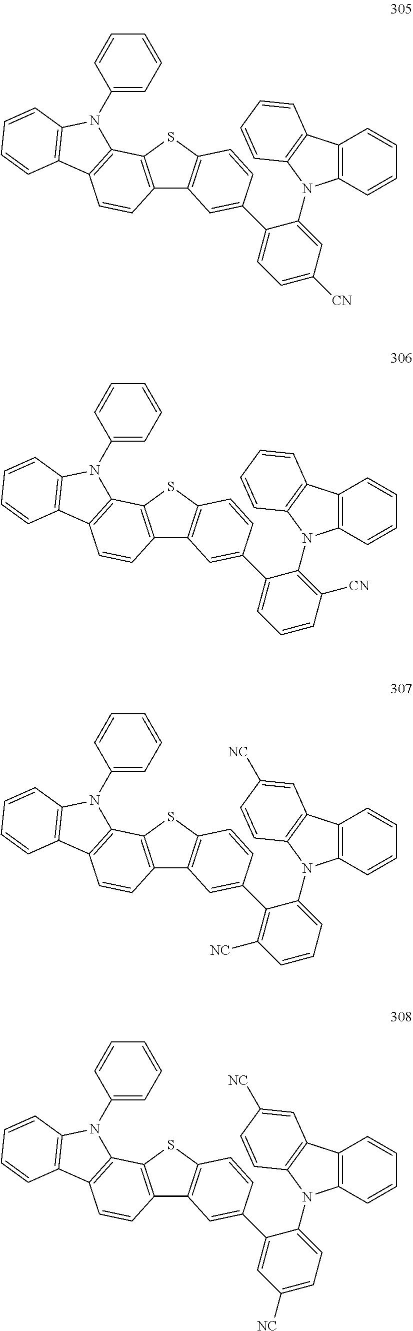 [SMILES: N#Cc1ccc(-c2ccc3sc4c(ccc5c6ccccc6n(-c6ccccc6)c54)c3c2)c(-n2c3ccccc3c3ccccc32)c1 ; N#Cc1cccc(-c2ccc3sc4c(ccc5c6ccccc6n(-c6ccccc6)c54)c3c2)c1-n1c2ccccc2c2ccccc21 ; N#Cc1ccc2c(c1)c1ccccc1n2-c1cccc(C#N)c1-c1ccc2sc3c(ccc4c5ccccc5n(-c5ccccc5)c43)c2c1 ; N#Cc1ccc(-n2c3ccccc3c3cc(C#N)ccc32)c(-c2ccc3sc4c(ccc5c6ccccc6n(-c6ccccc6)c54)c3c2)c1]

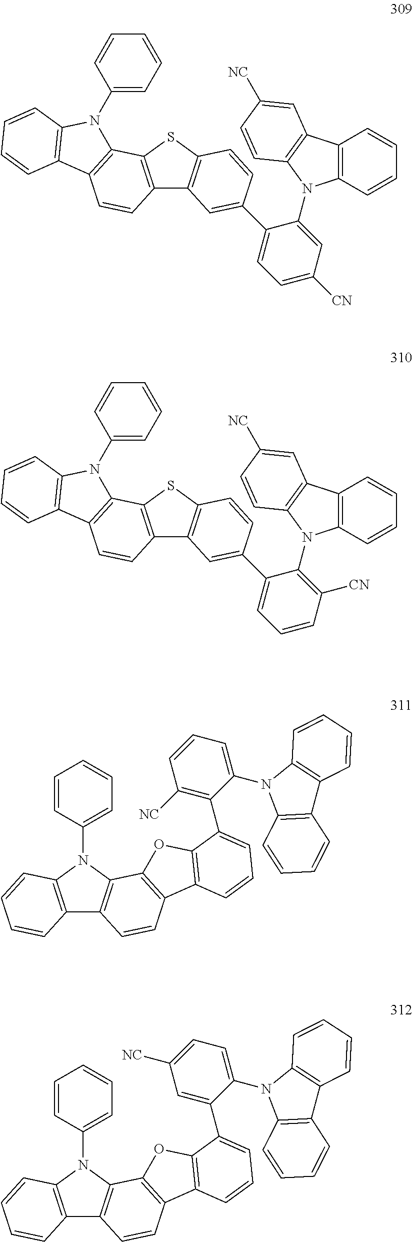

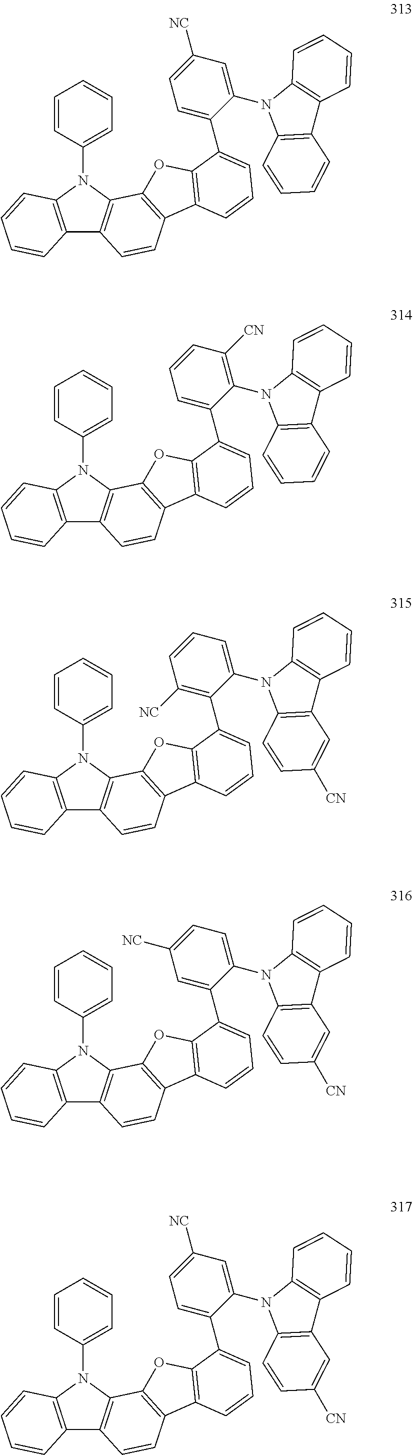

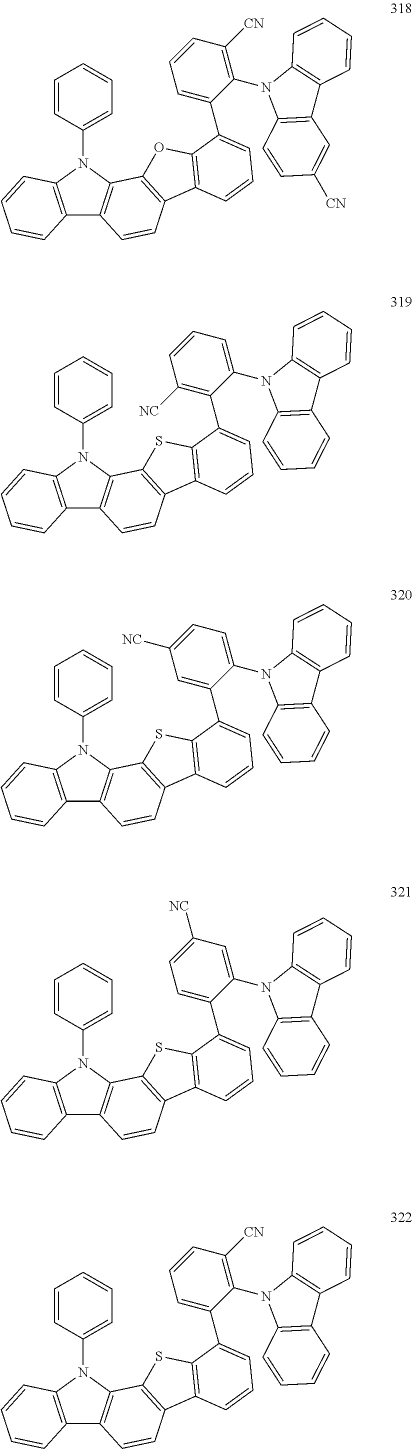

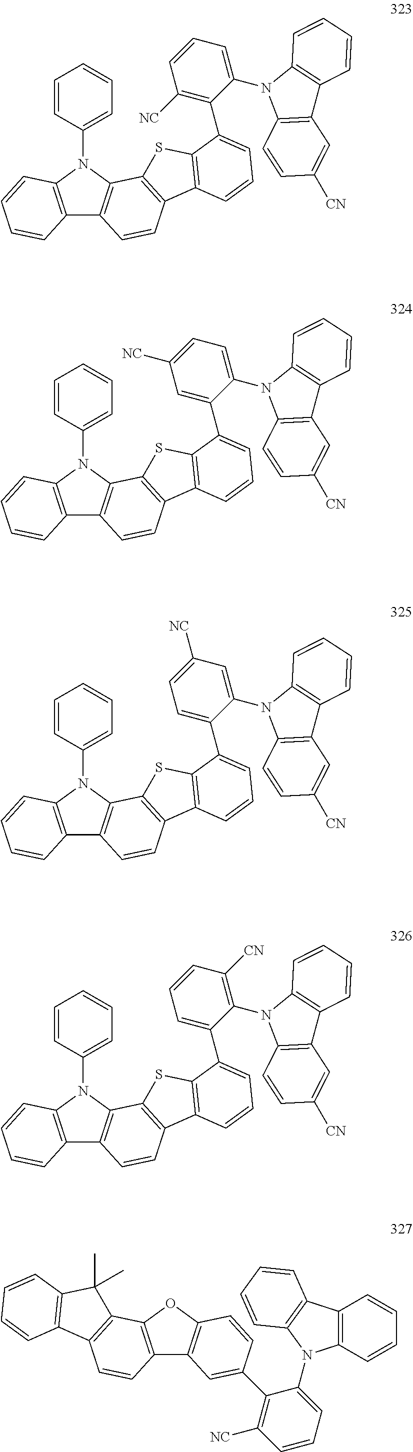

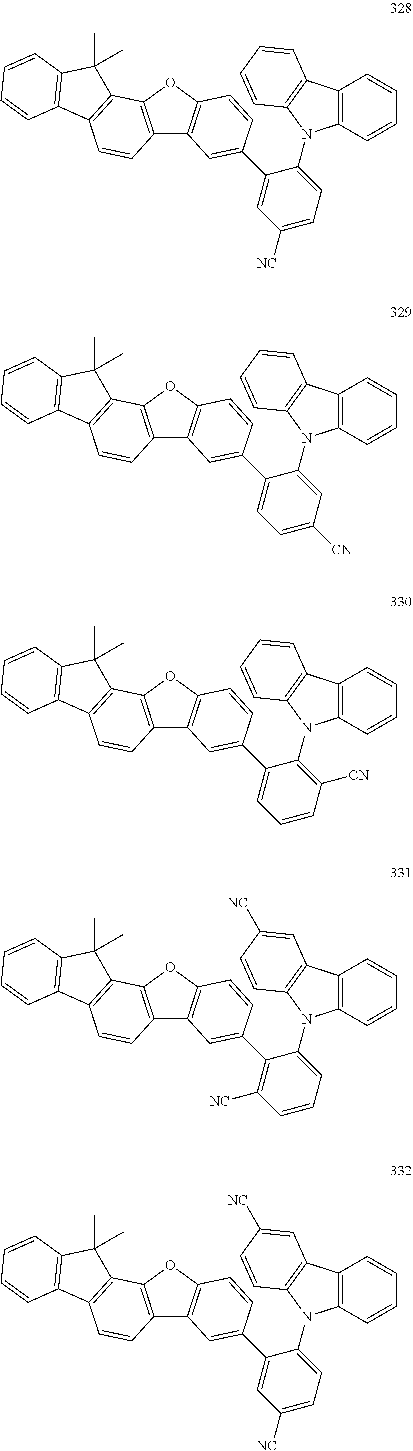

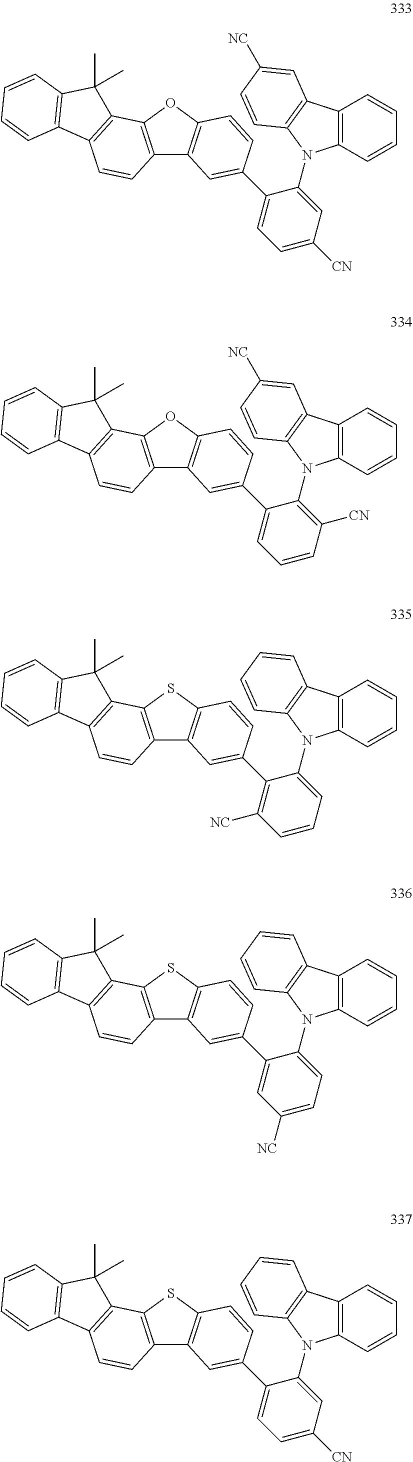

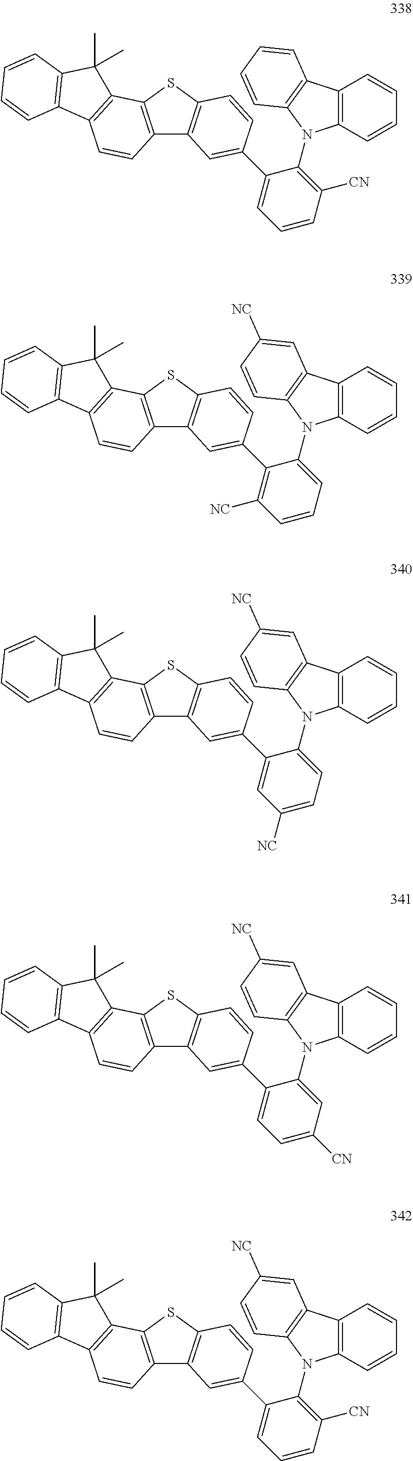

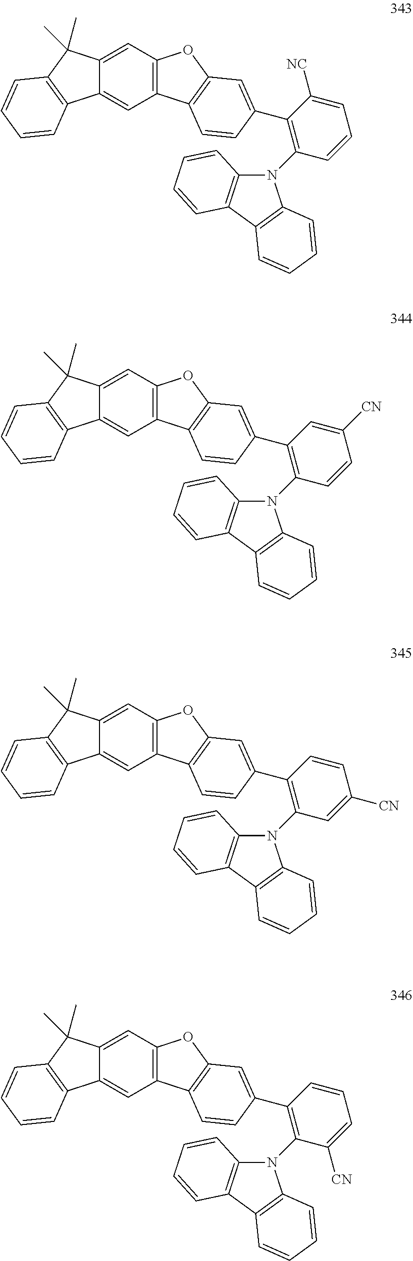 [SMILES: CC1(C)c2ccccc2-c2cc3c(cc21)oc1cc(-c2c(C#N)cccc2-n2c4ccccc4c4ccccc42)ccc13 ; CC1(C)c2ccccc2-c2cc3c(cc21)oc1cc(-c2cc(C#N)ccc2-n2c4ccccc4c4ccccc42)ccc13 ; CC1(C)c2ccccc2-c2cc3c(cc21)oc1cc(-c2ccc(C#N)cc2-n2c4ccccc4c4ccccc42)ccc13 ; CC1(C)c2ccccc2-c2cc3c(cc21)oc1cc(-c2cccc(C#N)c2-n2c4ccccc4c4ccccc42)ccc13]

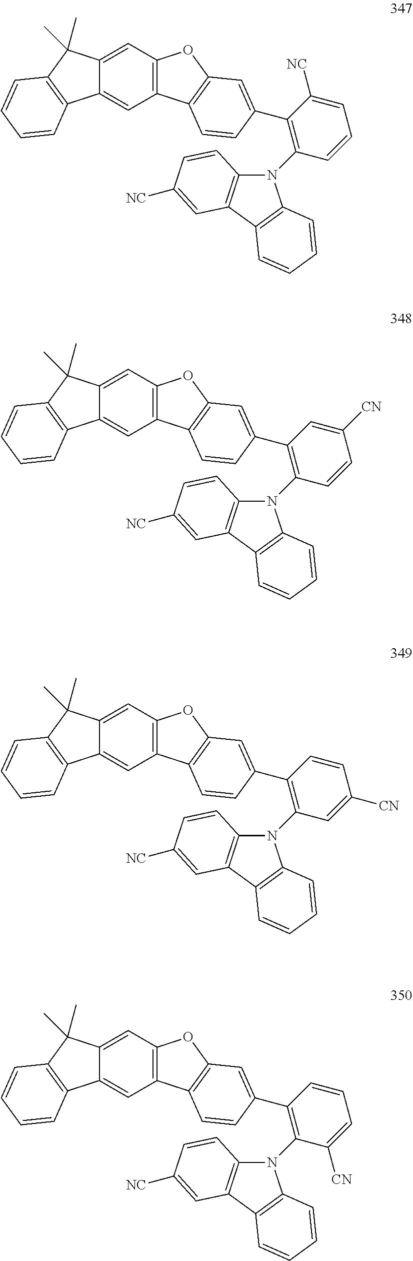 [SMILES: CC1(C)c2ccccc2-c2cc3c(cc21)oc1cc(-c2c(C#N)cccc2-n2c4ccccc4c4cc(C#N)ccc42)ccc13 ; CC1(C)c2ccccc2-c2cc3c(cc21)oc1cc(-c2cc(C#N)ccc2-n2c4ccccc4c4cc(C#N)ccc42)ccc13 ; CC1(C)c2ccccc2-c2cc3c(cc21)oc1cc(-c2ccc(C#N)cc2-n2c4ccccc4c4cc(C#N)ccc42)ccc13 ; CC1(C)c2ccccc2-c2cc3c(cc21)oc1cc(-c2cccc(C#N)c2-n2c4ccccc4c4cc(C#N)ccc42)ccc13]

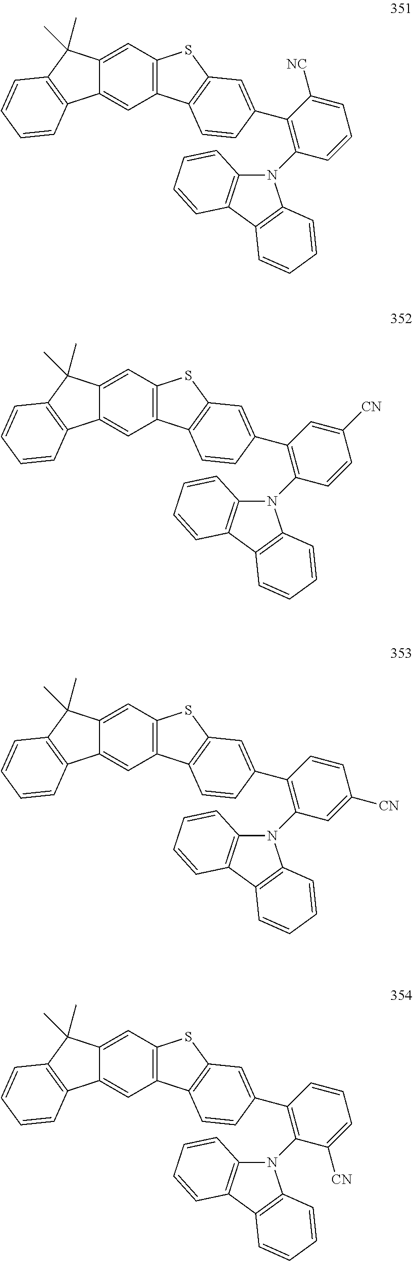 [SMILES: CC1(C)c2ccccc2-c2cc3c(cc21)sc1cc(-c2c(C#N)cccc2-n2c4ccccc4c4ccccc42)ccc13 ; CC1(C)c2ccccc2-c2cc3c(cc21)sc1cc(-c2cc(C#N)ccc2-n2c4ccccc4c4ccccc42)ccc13 ; CC1(C)c2ccccc2-c2cc3c(cc21)sc1cc(-c2ccc(C#N)cc2-n2c4ccccc4c4ccccc42)ccc13 ; CC1(C)c2ccccc2-c2cc3c(cc21)sc1cc(-c2cccc(C#N)c2-n2c4ccccc4c4ccccc42)ccc13]

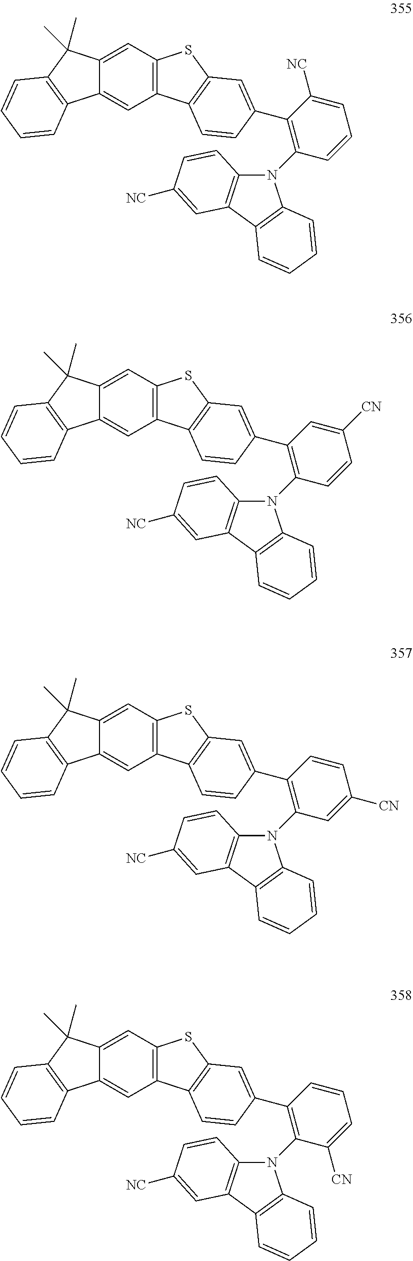 [SMILES: CC1(C)c2ccccc2-c2cc3c(cc21)sc1cc(-c2c(C#N)cccc2-n2c4ccccc4c4cc(C#N)ccc42)ccc13 ; CC1(C)c2ccccc2-c2cc3c(cc21)sc1cc(-c2cc(C#N)ccc2-n2c4ccccc4c4cc(C#N)ccc42)ccc13 ; CC1(C)c2ccccc2-c2cc3c(cc21)sc1cc(-c2ccc(C#N)cc2-n2c4ccccc4c4cc(C#N)ccc42)ccc13 ; CC1(C)c2ccccc2-c2cc3c(cc21)sc1cc(-c2cccc(C#N)c2-n2c4ccccc4c4cc(C#N)ccc42)ccc13]

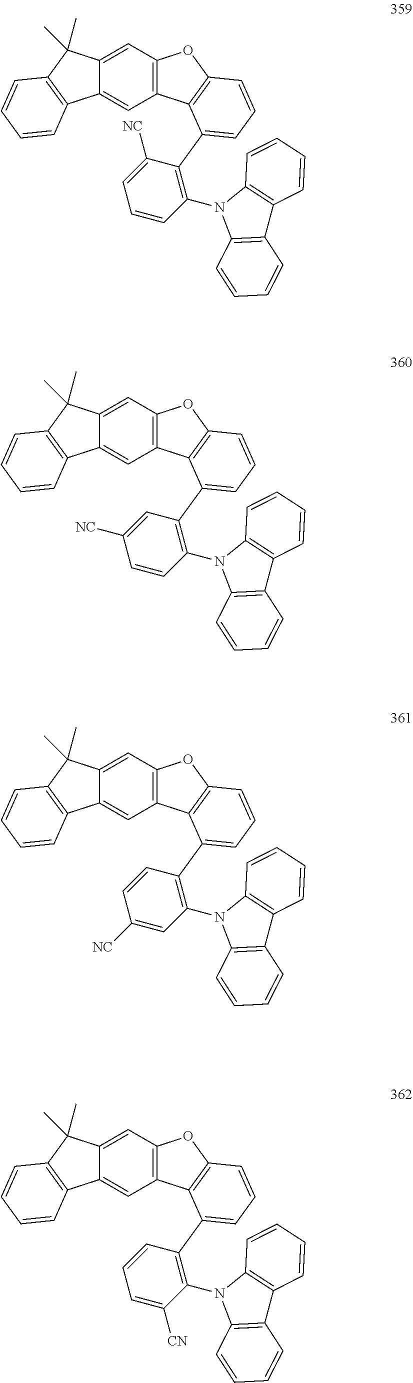 [SMILES: CC1(C)c2ccccc2-c2cc3c(cc21)oc1cccc(-c2c(C#N)cccc2-n2c4ccccc4c4ccccc42)c13 ; CC1(C)c2ccccc2-c2cc3c(cc21)oc1cccc(-c2cc(C#N)ccc2-n2c4ccccc4c4ccccc42)c13 ; CC1(C)c2ccccc2-c2cc3c(cc21)oc1cccc(-c2ccc(C#N)cc2-n2c4ccccc4c4ccccc42)c13 ; CC1(C)c2ccccc2-c2cc3c(cc21)oc1cccc(-c2cccc(C#N)c2-n2c4ccccc4c4ccccc42)c13]

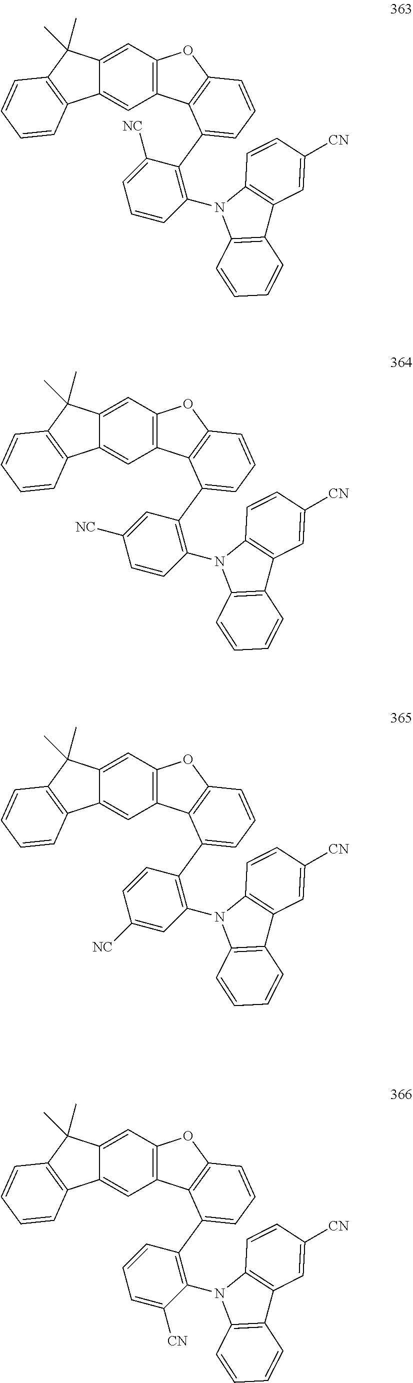 [SMILES: CC1(C)c2ccccc2-c2cc3c(cc21)oc1cccc(-c2c(C#N)cccc2-n2c4ccccc4c4cc(C#N)ccc42)c13 ; CC1(C)c2ccccc2-c2cc3c(cc21)oc1cccc(-c2cc(C#N)ccc2-n2c4ccccc4c4cc(C#N)ccc42)c13 ; CC1(C)c2ccccc2-c2cc3c(cc21)oc1cccc(-c2ccc(C#N)cc2-n2c4ccccc4c4cc(C#N)ccc42)c13 ; CC1(C)c2ccccc2-c2cc3c(cc21)oc1cccc(-c2cccc(C#N)c2-n2c4ccccc4c4cc(C#N)ccc42)c13]

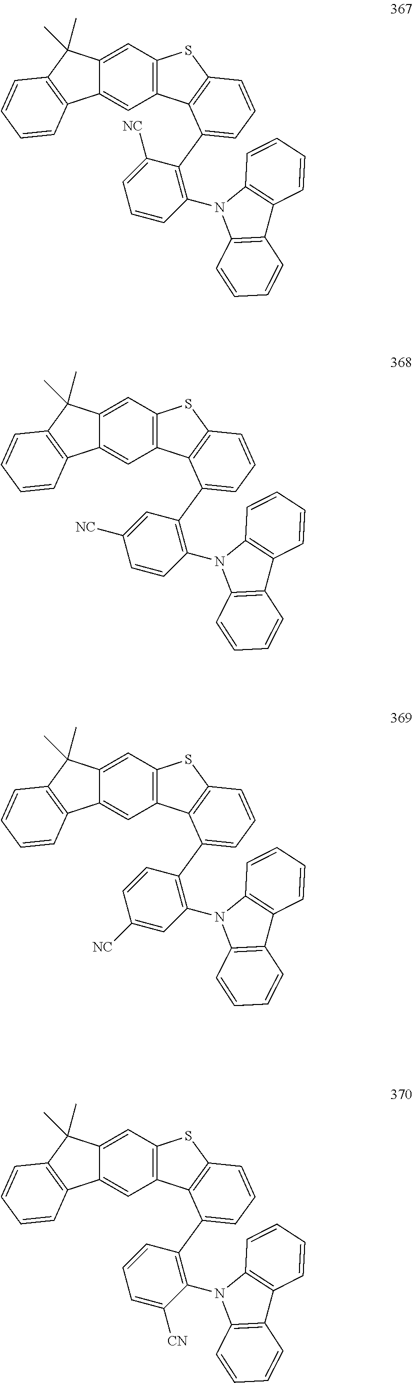 [SMILES: CC1(C)c2ccccc2-c2cc3c(cc21)sc1cccc(-c2c(C#N)cccc2-n2c4ccccc4c4ccccc42)c13 ; CC1(C)c2ccccc2-c2cc3c(cc21)sc1cccc(-c2cc(C#N)ccc2-n2c4ccccc4c4ccccc42)c13 ; CC1(C)c2ccccc2-c2cc3c(cc21)sc1cccc(-c2ccc(C#N)cc2-n2c4ccccc4c4ccccc42)c13 ; CC1(C)c2ccccc2-c2cc3c(cc21)sc1cccc(-c2cccc(C#N)c2-n2c4ccccc4c4ccccc42)c13]

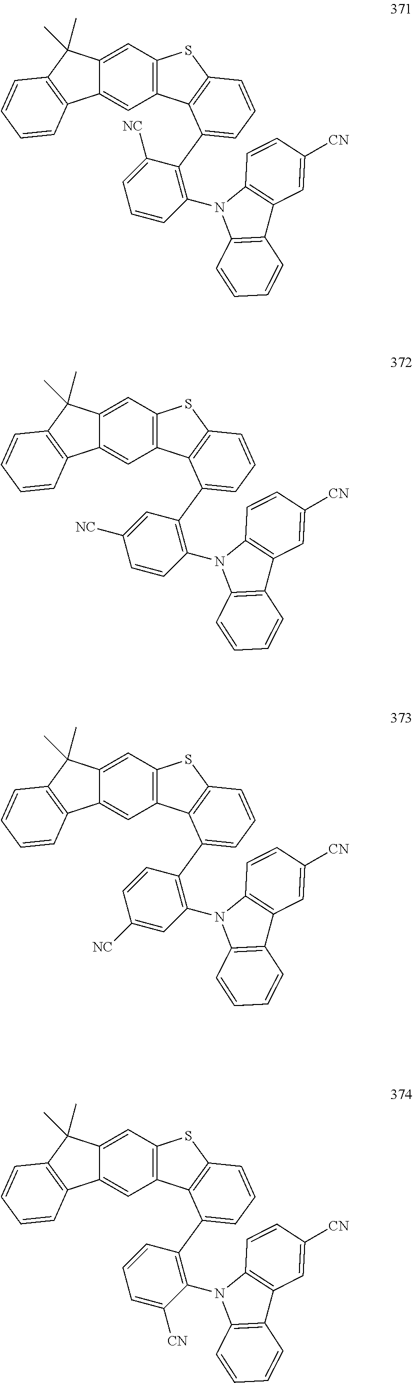 [SMILES: CC1(C)c2ccccc2-c2cc3c(cc21)sc1cccc(-c2c(C#N)cccc2-n2c4ccccc4c4cc(C#N)ccc42)c13 ; CC1(C)c2ccccc2-c2cc3c(cc21)sc1cccc(-c2cc(C#N)ccc2-n2c4ccccc4c4cc(C#N)ccc42)c13 ; CC1(C)c2ccccc2-c2cc3c(cc21)sc1cccc(-c2ccc(C#N)cc2-n2c4ccccc4c4cc(C#N)ccc42)c13 ; CC1(C)c2ccccc2-c2cc3c(cc21)sc1cccc(-c2cccc(C#N)c2-n2c4ccccc4c4cc(C#N)ccc42)c13]

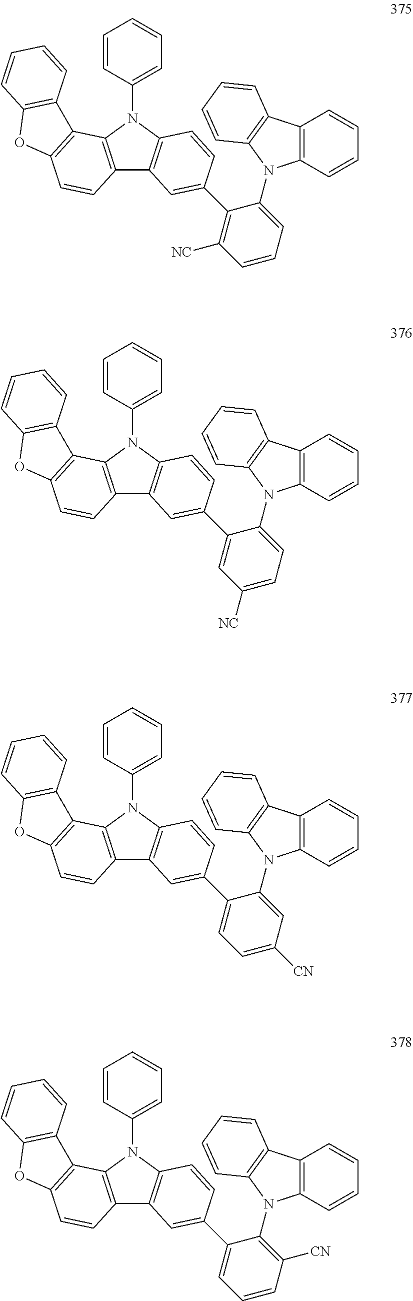 [SMILES: N#Cc1cccc(-n2c3ccccc3c3ccccc32)c1-c1ccc2c(c1)c1ccc3oc4ccccc4c3c1n2-c1ccccc1 ; N#Cc1ccc(-n2c3ccccc3c3ccccc32)c(-c2ccc3c(c2)c2ccc4oc5ccccc5c4c2n3-c2ccccc2)c1 ; N#Cc1ccc(-c2ccc3c(c2)c2ccc4oc5ccccc5c4c2n3-c2ccccc2)c(-n2c3ccccc3c3ccccc32)c1 ; N#Cc1cccc(-c2ccc3c(c2)c2ccc4oc5ccccc5c4c2n3-c2ccccc2)c1-n1c2ccccc2c2ccccc21]

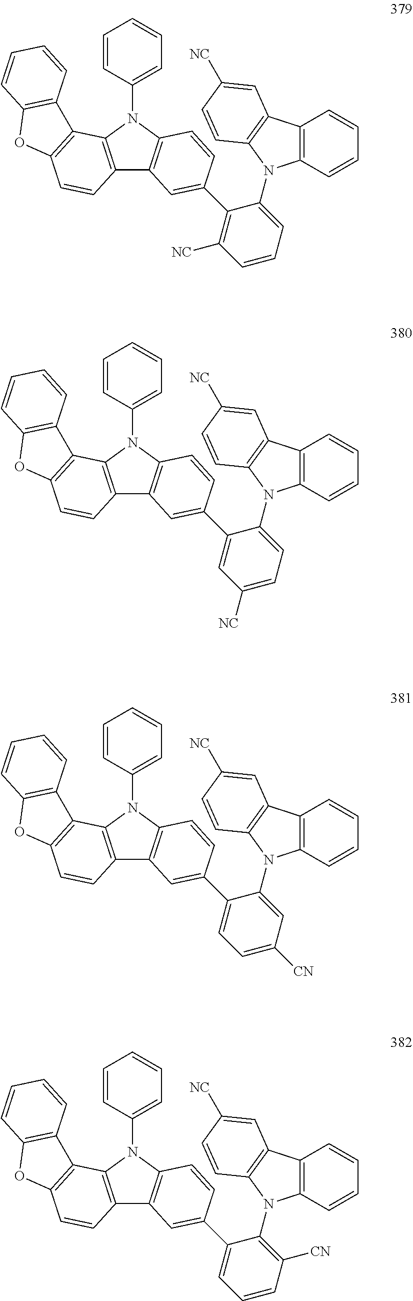 [SMILES: N#Cc1ccc2c(c1)c1ccccc1n2-c1cccc(C#N)c1-c1ccc2c(c1)c1ccc3oc4ccccc4c3c1n2-c1ccccc1 ; N#Cc1ccc(-n2c3ccccc3c3cc(C#N)ccc32)c(-c2ccc3c(c2)c2ccc4oc5ccccc5c4c2n3-c2ccccc2)c1 ; N#Cc1ccc(-c2ccc3c(c2)c2ccc4oc5ccccc5c4c2n3-c2ccccc2)c(-n2c3ccccc3c3cc(C#N)ccc32)c1 ; N#Cc1ccc2c(c1)c1ccccc1n2-c1c(C#N)cccc1-c1ccc2c(c1)c1ccc3oc4ccccc4c3c1n2-c1ccccc1]

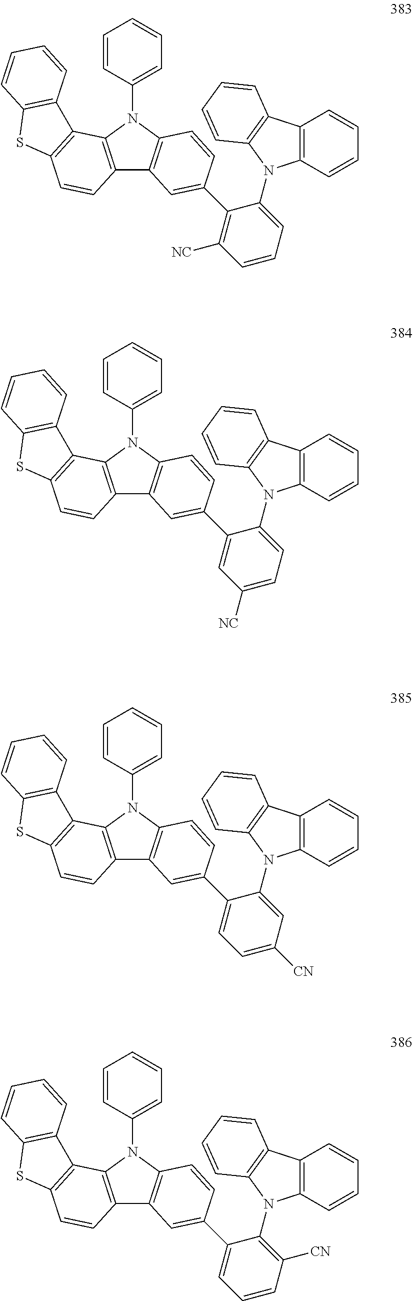 [SMILES: N#Cc1cccc(-n2c3ccccc3c3ccccc32)c1-c1ccc2c(c1)c1ccc3sc4ccccc4c3c1n2-c1ccccc1 ; N#Cc1ccc(-n2c3ccccc3c3ccccc32)c(-c2ccc3c(c2)c2ccc4sc5ccccc5c4c2n3-c2ccccc2)c1 ; N#Cc1ccc(-c2ccc3c(c2)c2ccc4sc5ccccc5c4c2n3-c2ccccc2)c(-n2c3ccccc3c3ccccc32)c1 ; N#Cc1cccc(-c2ccc3c(c2)c2ccc4sc5ccccc5c4c2n3-c2ccccc2)c1-n1c2ccccc2c2ccccc21]

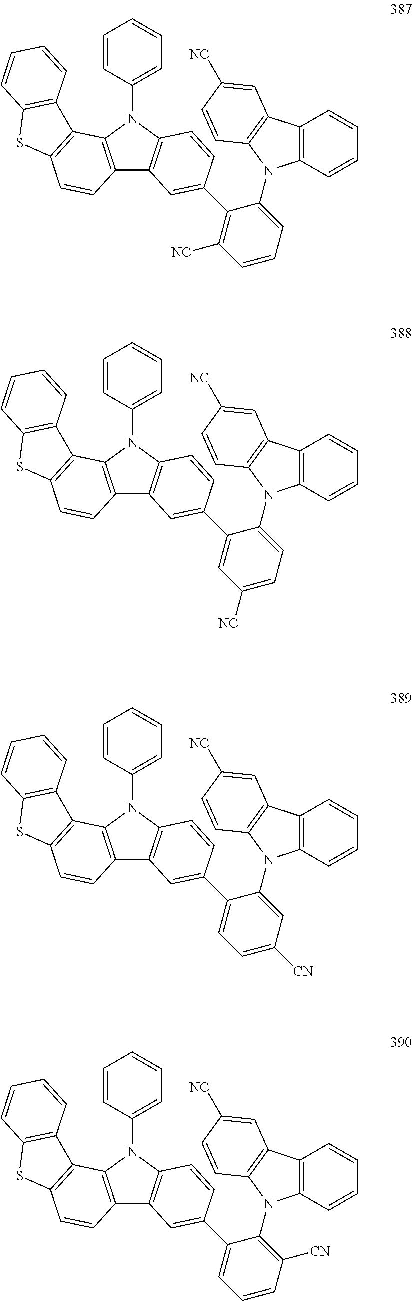 [SMILES: N#Cc1ccc2c(c1)c1ccccc1n2-c1cccc(C#N)c1-c1ccc2c(c1)c1ccc3sc4ccccc4c3c1n2-c1ccccc1 ; N#Cc1ccc(-n2c3ccccc3c3cc(C#N)ccc32)c(-c2ccc3c(c2)c2ccc4sc5ccccc5c4c2n3-c2ccccc2)c1 ; N#Cc1ccc(-c2ccc3c(c2)c2ccc4sc5ccccc5c4c2n3-c2ccccc2)c(-n2c3ccccc3c3cc(C#N)ccc32)c1 ; N#Cc1ccc2c(c1)c1ccccc1n2-c1c(C#N)cccc1-c1ccc2c(c1)c1ccc3sc4ccccc4c3c1n2-c1ccccc1]

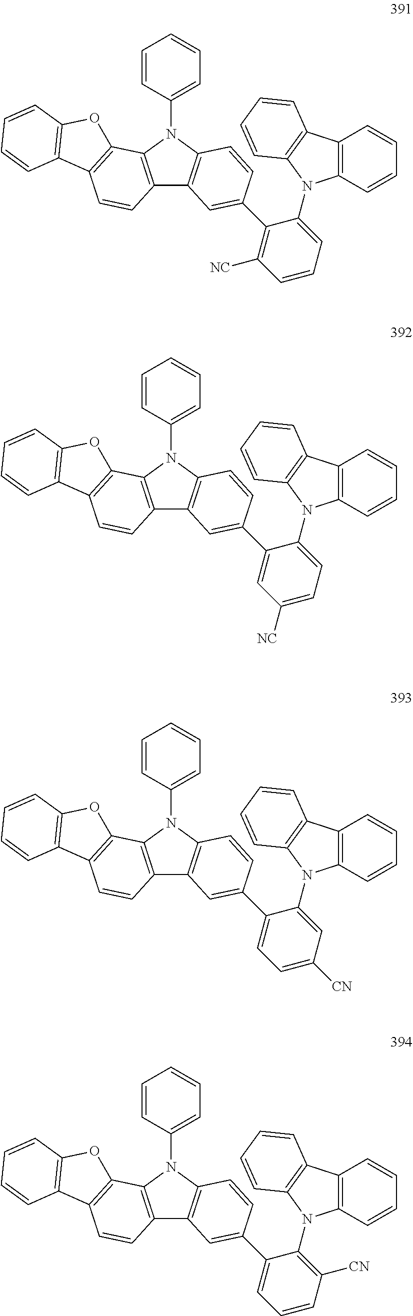 [SMILES: N#Cc1cccc(-n2c3ccccc3c3ccccc32)c1-c1ccc2c(c1)c1ccc3c4ccccc4oc3c1n2-c1ccccc1 ; N#Cc1ccc(-n2c3ccccc3c3ccccc32)c(-c2ccc3c(c2)c2ccc4c5ccccc5oc4c2n3-c2ccccc2)c1 ; N#Cc1ccc(-c2ccc3c(c2)c2ccc4c5ccccc5oc4c2n3-c2ccccc2)c(-n2c3ccccc3c3ccccc32)c1 ; N#Cc1cccc(-c2ccc3c(c2)c2ccc4c5ccccc5oc4c2n3-c2ccccc2)c1-n1c2ccccc2c2ccccc21]

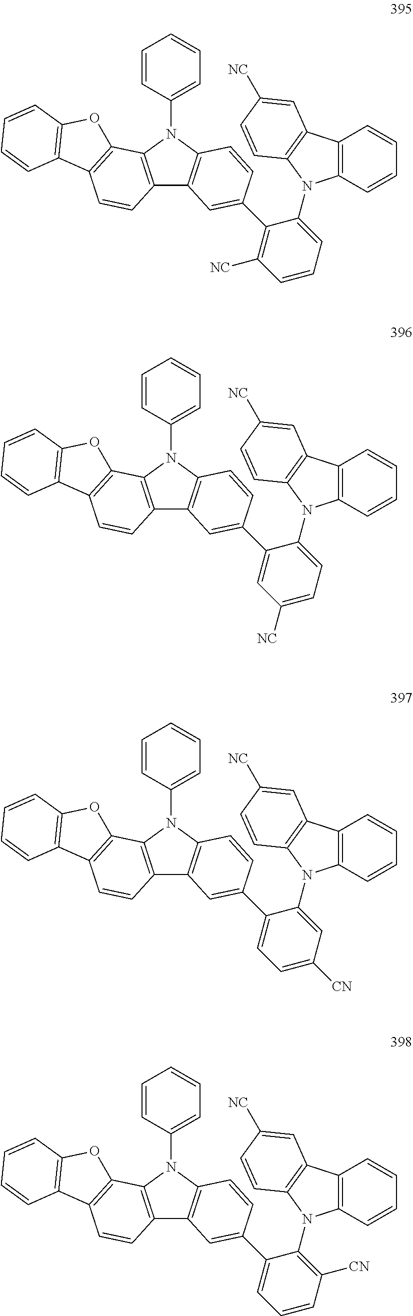 [SMILES: N#Cc1ccc2c(c1)c1ccccc1n2-c1cccc(C#N)c1-c1ccc2c(c1)c1ccc3c4ccccc4oc3c1n2-c1ccccc1 ; N#Cc1ccc(-n2c3ccccc3c3cc(C#N)ccc32)c(-c2ccc3c(c2)c2ccc4c5ccccc5oc4c2n3-c2ccccc2)c1 ; N#Cc1ccc(-c2ccc3c(c2)c2ccc4c5ccccc5oc4c2n3-c2ccccc2)c(-n2c3ccccc3c3cc(C#N)ccc32)c1 ; N#Cc1ccc2c(c1)c1ccccc1n2-c1c(C#N)cccc1-c1ccc2c(c1)c1ccc3c4ccccc4oc3c1n2-c1ccccc1]

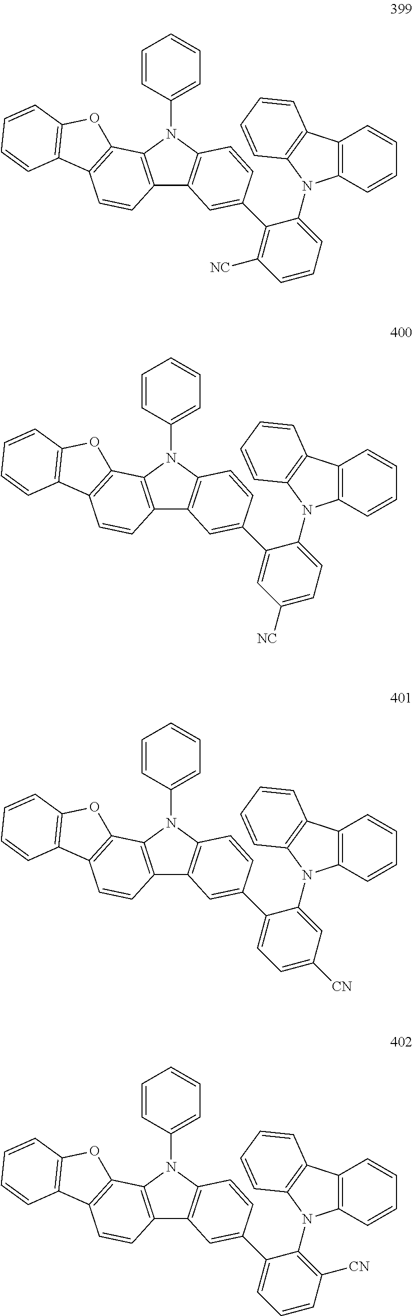 [SMILES: N#Cc1cccc(-n2c3ccccc3c3ccccc32)c1-c1ccc2c(c1)c1ccc3c4ccccc4oc3c1n2-c1ccccc1 ; N#Cc1ccc(-n2c3ccccc3c3ccccc32)c(-c2ccc3c(c2)c2ccc4c5ccccc5oc4c2n3-c2ccccc2)c1 ; N#Cc1ccc(-c2ccc3c(c2)c2ccc4c5ccccc5oc4c2n3-c2ccccc2)c(-n2c3ccccc3c3ccccc32)c1 ; N#Cc1cccc(-c2ccc3c(c2)c2ccc4c5ccccc5oc4c2n3-c2ccccc2)c1-n1c2ccccc2c2ccccc21]

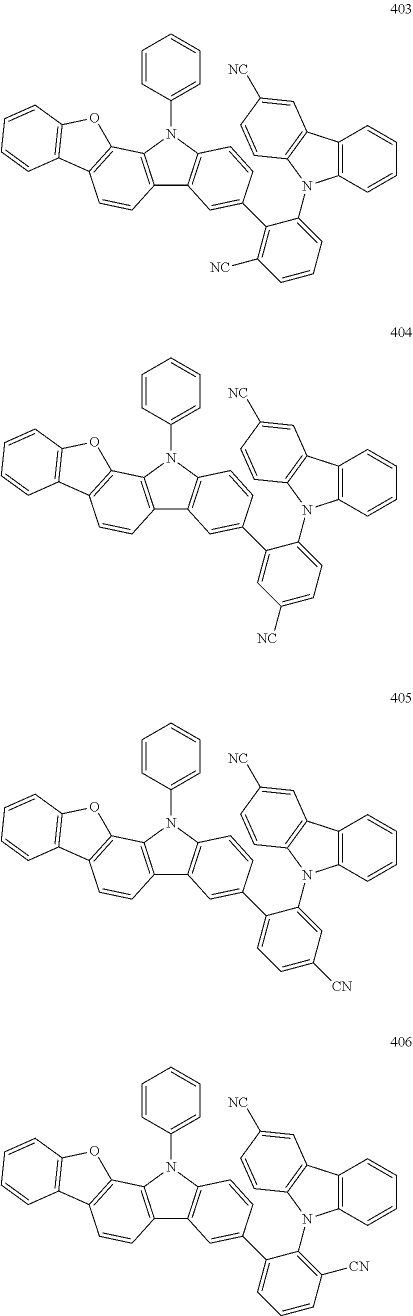 [SMILES: N#Cc1ccc2c(c1)c1ccccc1n2-c1cccc(C#N)c1-c1ccc2c(c1)c1ccc3c4ccccc4oc3c1n2-c1ccccc1 ; N#Cc1ccc(-n2c3ccccc3c3cc(C#N)ccc32)c(-c2ccc3c(c2)c2ccc4c5ccccc5oc4c2n3-c2ccccc2)c1 ; N#Cc1ccc(-c2ccc3c(c2)c2ccc4c5ccccc5oc4c2n3-c2ccccc2)c(-n2c3ccccc3c3cc(C#N)ccc32)c1 ; N#Cc1ccc2c(c1)c1ccccc1n2-c1c(C#N)cccc1-c1ccc2c(c1)c1ccc3c4ccccc4oc3c1n2-c1ccccc1]

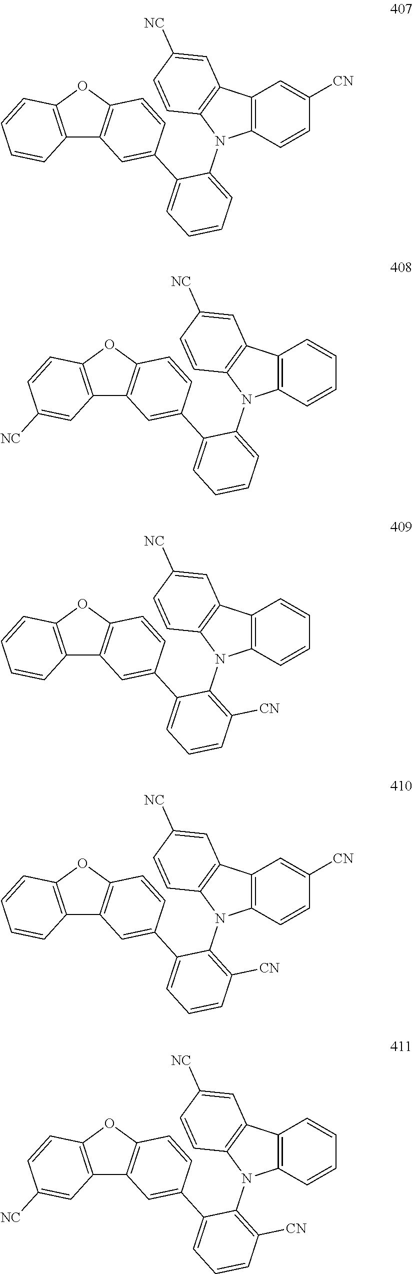

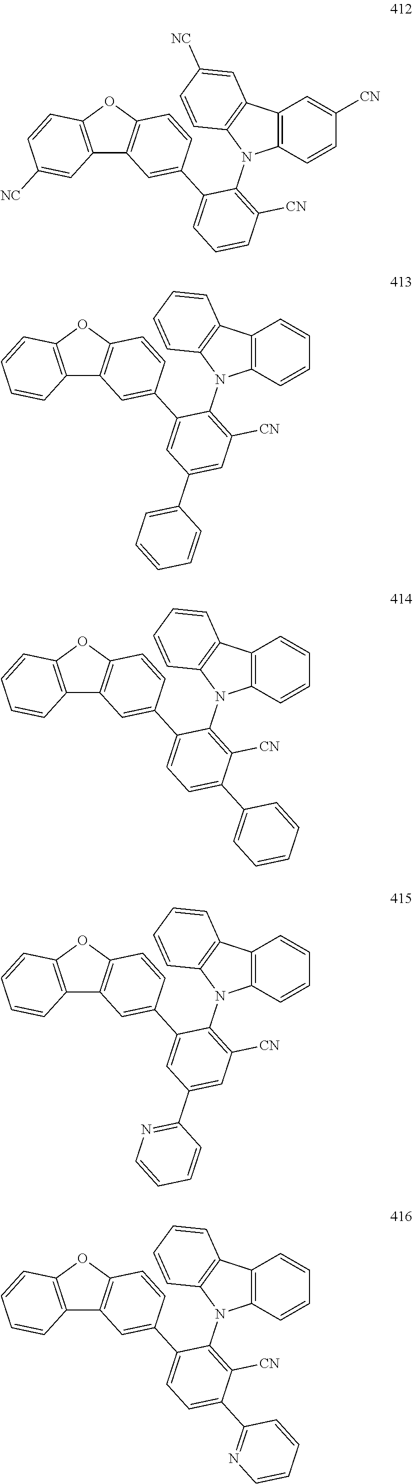

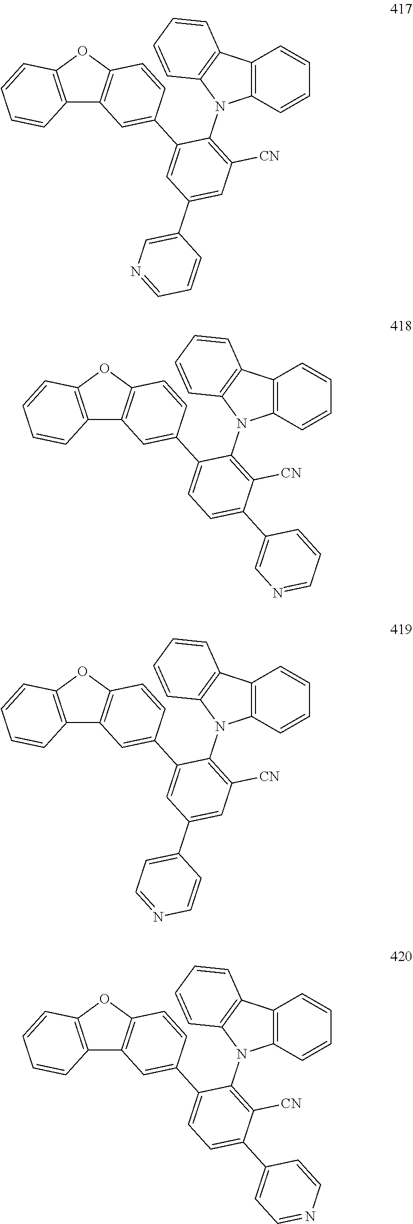

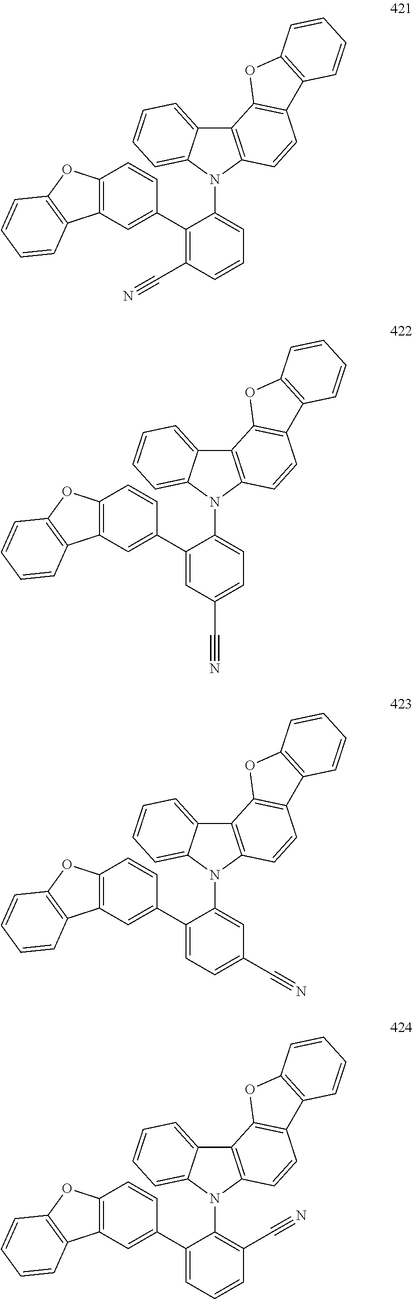 [SMILES: N#Cc1cccc(-n2c3ccccc3c3c4oc5ccccc5c4ccc32)c1-c1ccc2oc3ccccc3c2c1 ; N#Cc1ccc(-n2c3ccccc3c3c4oc5ccccc5c4ccc32)c(-c2ccc3oc4ccccc4c3c2)c1 ; N#Cc1ccc(-c2ccc3oc4ccccc4c3c2)c(-n2c3ccccc3c3c4oc5ccccc5c4ccc32)c1 ; N#Cc1cccc(-c2ccc3oc4ccccc4c3c2)c1-n1c2ccccc2c2c3oc4ccccc4c3ccc21]

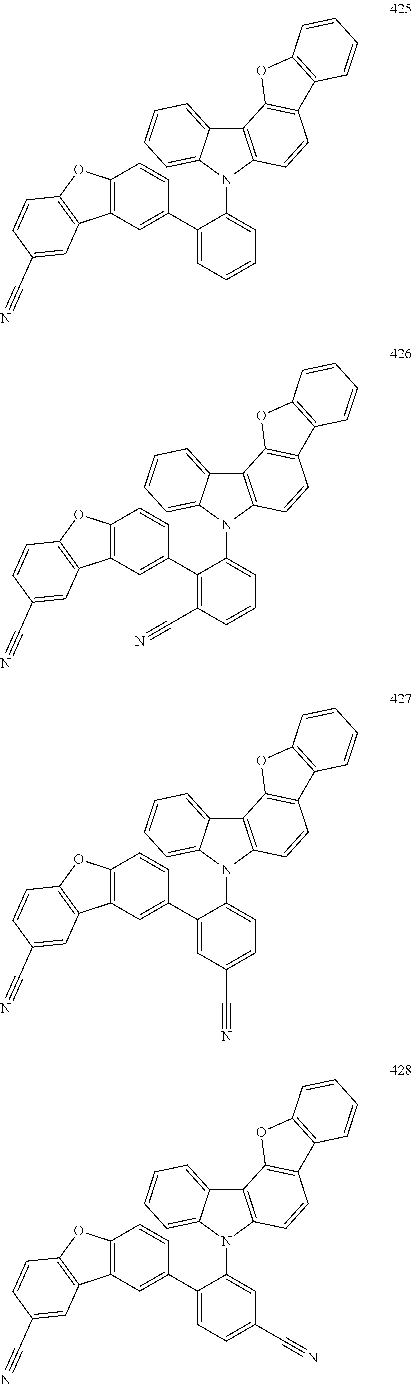 [SMILES: N#Cc1ccc2oc3ccc(-c4ccccc4-n4c5ccccc5c5c6oc7ccccc7c6ccc54)cc3c2c1 ; N#Cc1ccc2oc3ccc(-c4c(C#N)cccc4-n4c5ccccc5c5c6oc7ccccc7c6ccc54)cc3c2c1 ; N#Cc1ccc(-n2c3ccccc3c3c4oc5ccccc5c4ccc32)c(-c2ccc3oc4ccc(C#N)cc4c3c2)c1 ; N#Cc1ccc(-c2ccc3oc4ccc(C#N)cc4c3c2)c(-n2c3ccccc3c3c4oc5ccccc5c4ccc32)c1]

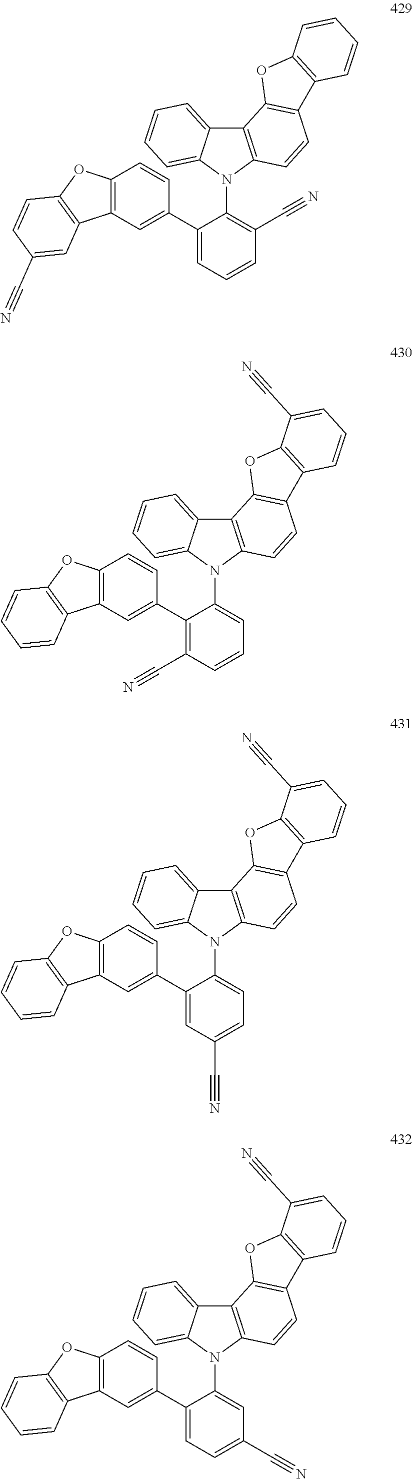 [SMILES: N#Cc1ccc2oc3ccc(-c4cccc(C#N)c4-n4c5ccccc5c5c6oc7ccccc7c6ccc54)cc3c2c1 ; N#Cc1cccc(-n2c3ccccc3c3c4oc5c(C#N)cccc5c4ccc32)c1-c1ccc2oc3ccccc3c2c1 ; N#Cc1ccc(-n2c3ccccc3c3c4oc5c(C#N)cccc5c4ccc32)c(-c2ccc3oc4ccccc4c3c2)c1 ; N#Cc1ccc(-c2ccc3oc4ccccc4c3c2)c(-n2c3ccccc3c3c4oc5c(C#N)cccc5c4ccc32)c1]

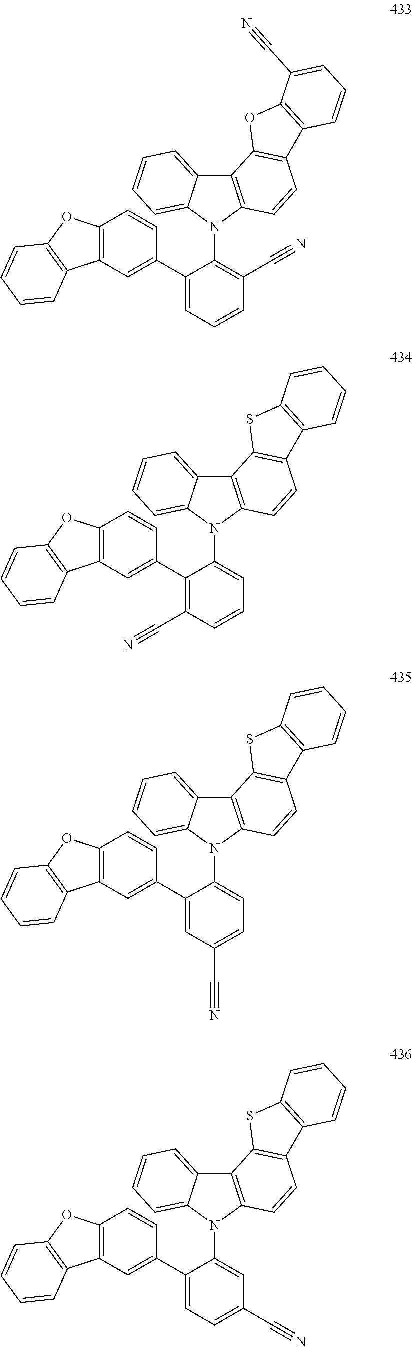 [SMILES: N#Cc1cccc(-c2ccc3oc4ccccc4c3c2)c1-n1c2ccccc2c2c3oc4c(C#N)cccc4c3ccc21 ; N#Cc1cccc(-n2c3ccccc3c3c4sc5ccccc5c4ccc32)c1-c1ccc2oc3ccccc3c2c1 ; N#Cc1ccc(-n2c3ccccc3c3c4sc5ccccc5c4ccc32)c(-c2ccc3oc4ccccc4c3c2)c1 ; N#Cc1ccc(-c2ccc3oc4ccccc4c3c2)c(-n2c3ccccc3c3c4sc5ccccc5c4ccc32)c1]

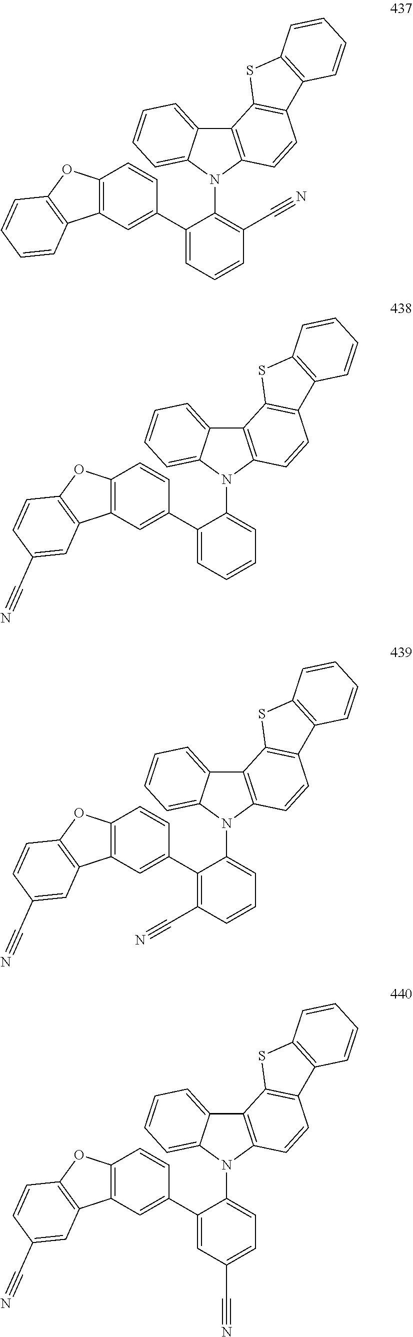 [SMILES: N#Cc1cccc(-c2ccc3oc4ccccc4c3c2)c1-n1c2ccccc2c2c3sc4ccccc4c3ccc21 ; N#Cc1ccc2oc3ccc(-c4ccccc4-n4c5ccccc5c5c6sc7ccccc7c6ccc54)cc3c2c1 ; N#Cc1ccc2oc3ccc(-c4c(C#N)cccc4-n4c5ccccc5c5c6sc7ccccc7c6ccc54)cc3c2c1 ; N#Cc1ccc(-n2c3ccccc3c3c4sc5ccccc5c4ccc32)c(-c2ccc3oc4ccc(C#N)cc4c3c2)c1]

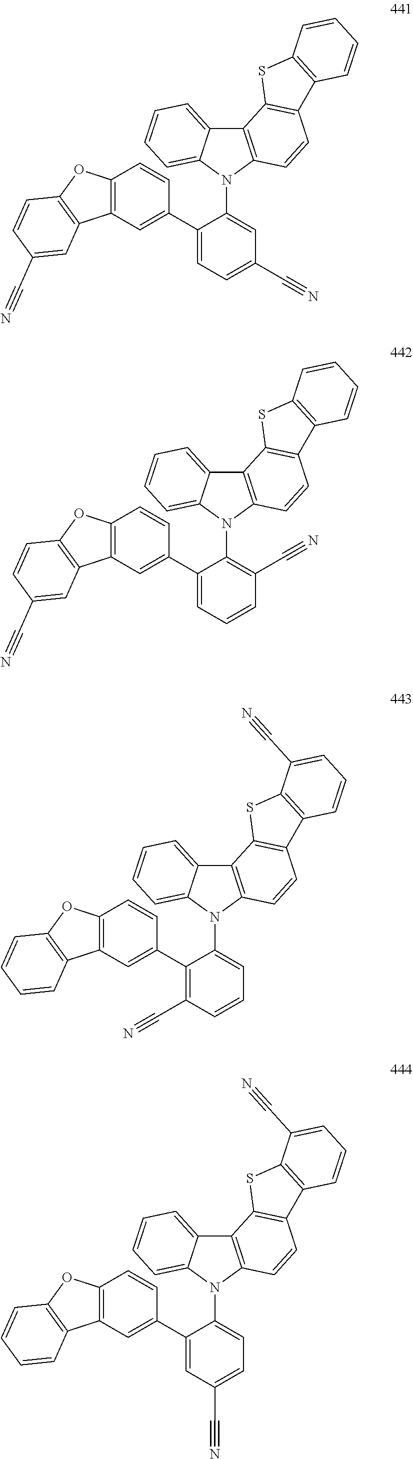 [SMILES: N#Cc1ccc(-c2ccc3oc4ccc(C#N)cc4c3c2)c(-n2c3ccccc3c3c4sc5ccccc5c4ccc32)c1 ; N#Cc1ccc2oc3ccc(-c4cccc(C#N)c4-n4c5ccccc5c5c6sc7ccccc7c6ccc54)cc3c2c1 ; N#Cc1cccc(-n2c3ccccc3c3c4sc5c(C#N)cccc5c4ccc32)c1-c1ccc2oc3ccccc3c2c1 ; N#Cc1ccc(-n2c3ccccc3c3c4sc5c(C#N)cccc5c4ccc32)c(-c2ccc3oc4ccccc4c3c2)c1]

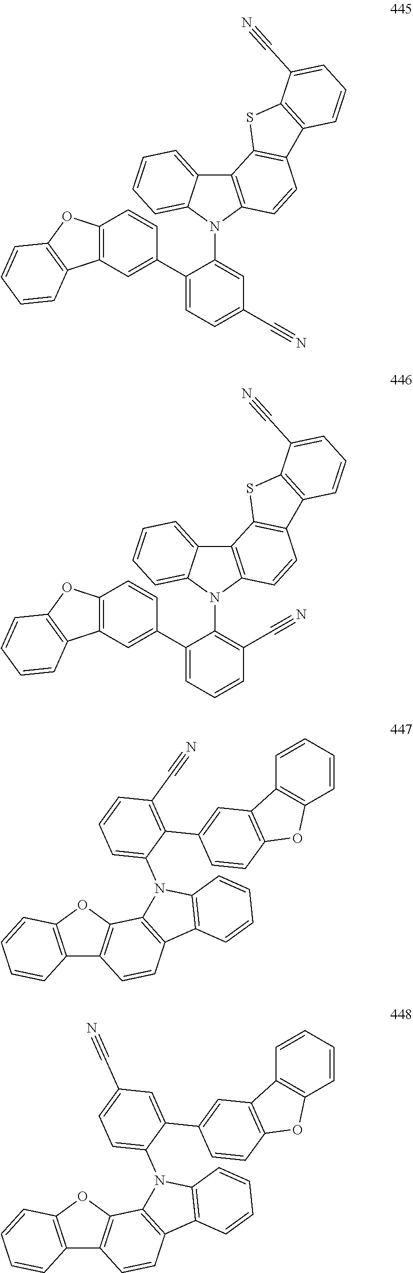 [SMILES: N#Cc1ccc(-c2ccc3oc4ccccc4c3c2)c(-n2c3ccccc3c3c4sc5c(C#N)cccc5c4ccc32)c1 ; N#Cc1cccc(-c2ccc3oc4ccccc4c3c2)c1-n1c2ccccc2c2c3sc4c(C#N)cccc4c3ccc21 ; N#Cc1cccc(-n2c3ccccc3c3ccc4c5ccccc5oc4c32)c1-c1ccc2oc3ccccc3c2c1 ; N#Cc1ccc(-n2c3ccccc3c3ccc4c5ccccc5oc4c32)c(-c2ccc3oc4ccccc4c3c2)c1]

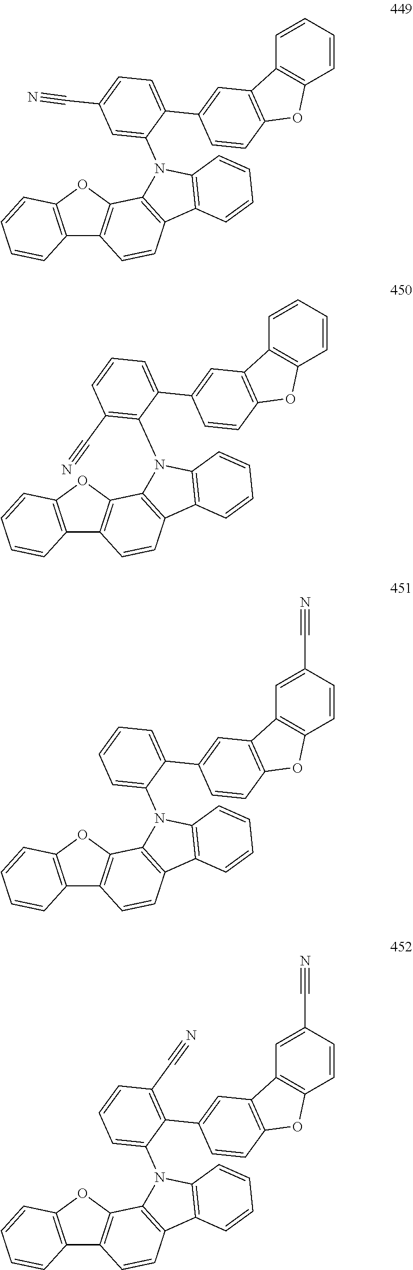 [SMILES: N#Cc1ccc(-c2ccc3oc4ccccc4c3c2)c(-n2c3ccccc3c3ccc4c5ccccc5oc4c32)c1 ; N#Cc1cccc(-c2ccc3oc4ccccc4c3c2)c1-n1c2ccccc2c2ccc3c4ccccc4oc3c21 ; N#Cc1ccc2oc3ccc(-c4ccccc4-n4c5ccccc5c5ccc6c7ccccc7oc6c54)cc3c2c1 ; N#Cc1ccc2oc3ccc(-c4c(C#N)cccc4-n4c5ccccc5c5ccc6c7ccccc7oc6c54)cc3c2c1]

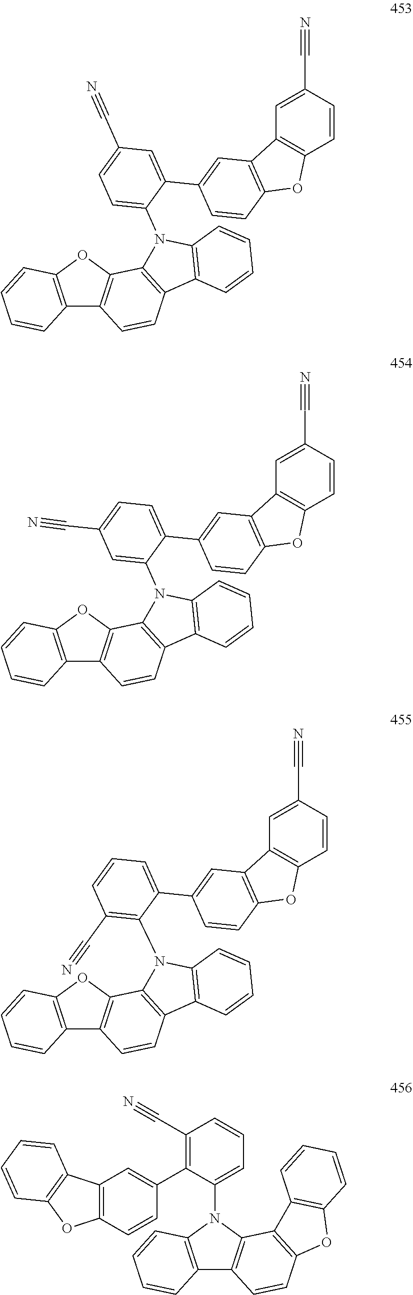 [SMILES: N#Cc1ccc(-n2c3ccccc3c3ccc4c5ccccc5oc4c32)c(-c2ccc3oc4ccc(C#N)cc4c3c2)c1 ; N#Cc1ccc(-c2ccc3oc4ccc(C#N)cc4c3c2)c(-n2c3ccccc3c3ccc4c5ccccc5oc4c32)c1 ; N#Cc1ccc2oc3ccc(-c4cccc(C#N)c4-n4c5ccccc5c5ccc6c7ccccc7oc6c54)cc3c2c1 ; N#Cc1cccc(-n2c3ccccc3c3ccc4oc5ccccc5c4c32)c1-c1ccc2oc3ccccc3c2c1]

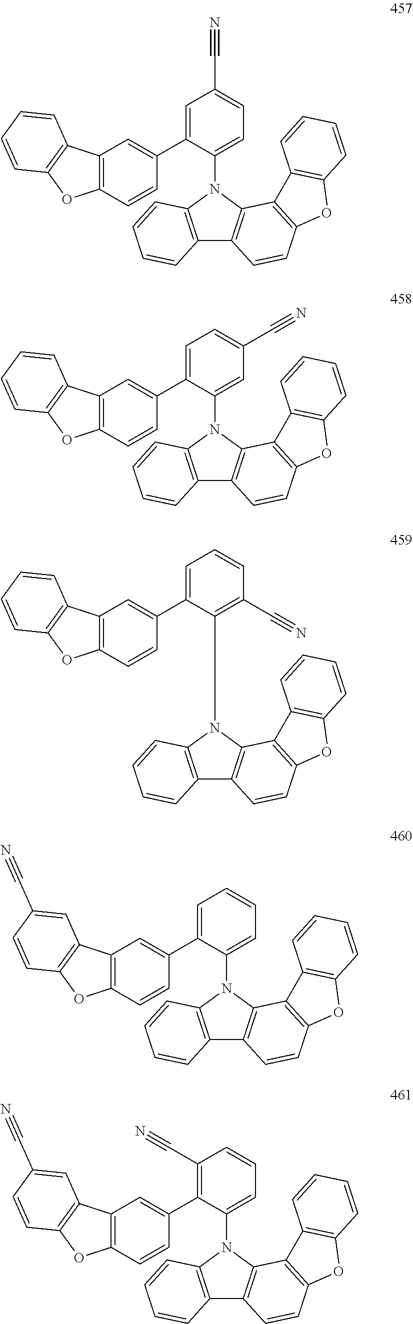 [SMILES: N#Cc1ccc(-n2c3ccccc3c3ccc4oc5ccccc5c4c32)c(-c2ccc3oc4ccccc4c3c2)c1 ; N#Cc1ccc(-c2ccc3oc4ccccc4c3c2)c(-n2c3ccccc3c3ccc4oc5ccccc5c4c32)c1 ; N#Cc1cccc(-c2ccc3oc4ccccc4c3c2)c1-n1c2ccccc2c2ccc3oc4ccccc4c3c21 ; N#Cc1ccc2oc3ccc(-c4ccccc4-n4c5ccccc5c5ccc6oc7ccccc7c6c54)cc3c2c1 ; N#Cc1ccc2oc3ccc(-c4c(C#N)cccc4-n4c5ccccc5c5ccc6oc7ccccc7c6c54)cc3c2c1]

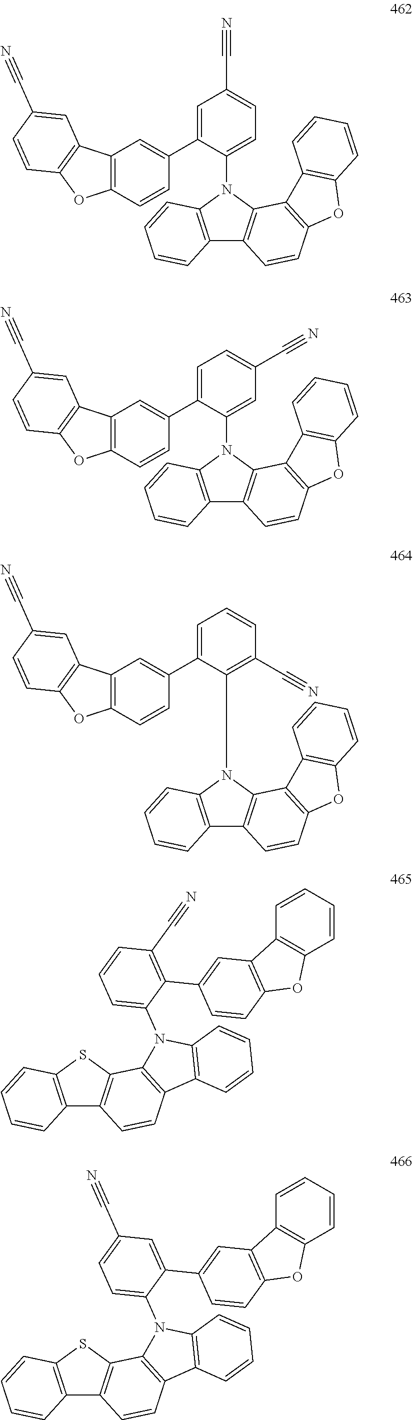

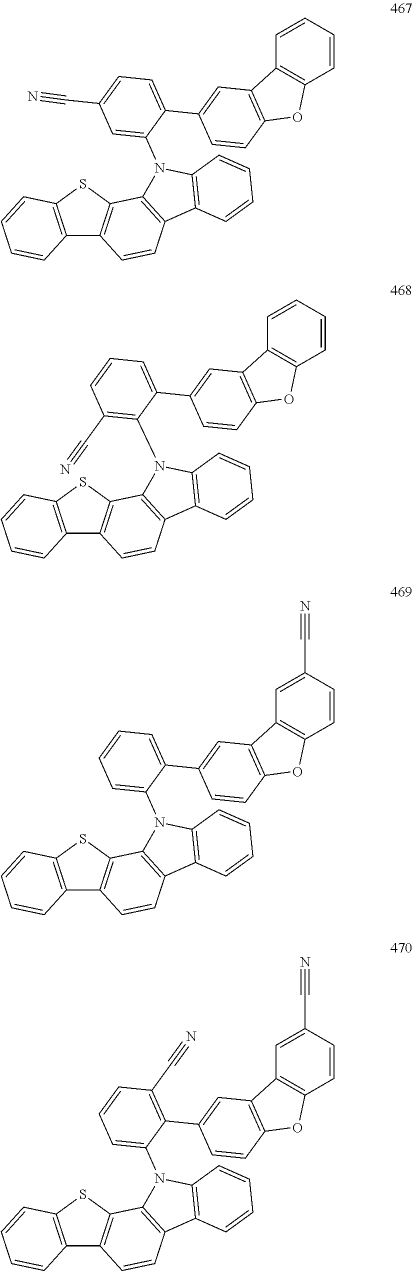 [SMILES: N#Cc1ccc(-c2ccc3oc4ccccc4c3c2)c(-n2c3ccccc3c3ccc4c5ccccc5sc4c32)c1 ; N#Cc1cccc(-c2ccc3oc4ccccc4c3c2)c1-n1c2ccccc2c2ccc3c4ccccc4sc3c21 ; N#Cc1ccc2oc3ccc(-c4ccccc4-n4c5ccccc5c5ccc6c7ccccc7sc6c54)cc3c2c1 ; N#Cc1ccc2oc3ccc(-c4c(C#N)cccc4-n4c5ccccc5c5ccc6c7ccccc7sc6c54)cc3c2c1]

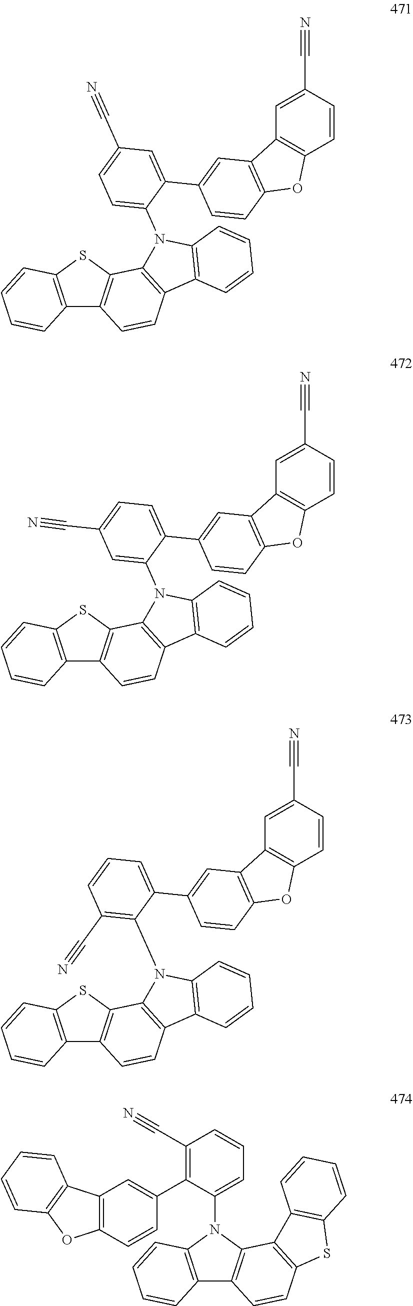 [SMILES: N#Cc1ccc(-n2c3ccccc3c3ccc4c5ccccc5sc4c32)c(-c2ccc3oc4ccc(C#N)cc4c3c2)c1 ; N#Cc1ccc(-c2ccc3oc4ccc(C#N)cc4c3c2)c(-n2c3ccccc3c3ccc4c5ccccc5sc4c32)c1 ; N#Cc1ccc2oc3ccc(-c4cccc(C#N)c4-n4c5ccccc5c5ccc6c7ccccc7sc6c54)cc3c2c1 ; N#Cc1cccc(-n2c3ccccc3c3ccc4sc5ccccc5c4c32)c1-c1ccc2oc3ccccc3c2c1]

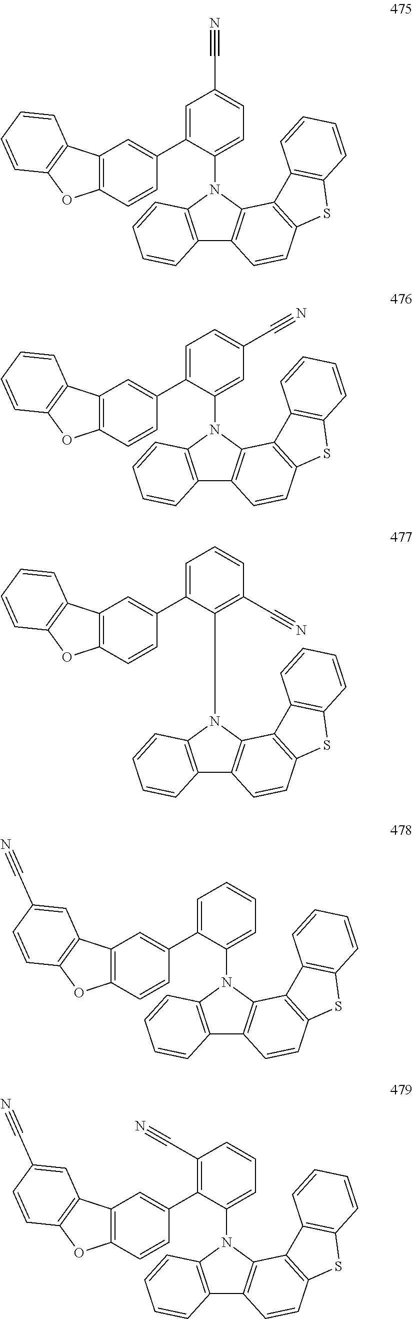 [SMILES: N#Cc1ccc(-n2c3ccccc3c3ccc4sc5ccccc5c4c32)c(-c2ccc3oc4ccccc4c3c2)c1 ; N#Cc1ccc(-c2ccc3oc4ccccc4c3c2)c(-n2c3ccccc3c3ccc4sc5ccccc5c4c32)c1 ; N#Cc1cccc(-c2ccc3oc4ccccc4c3c2)c1-n1c2ccccc2c2ccc3sc4ccccc4c3c21 ; N#Cc1ccc2oc3ccc(-c4ccccc4-n4c5ccccc5c5ccc6sc7ccccc7c6c54)cc3c2c1 ; N#Cc1ccc2oc3ccc(-c4c(C#N)cccc4-n4c5ccccc5c5ccc6sc7ccccc7c6c54)cc3c2c1]

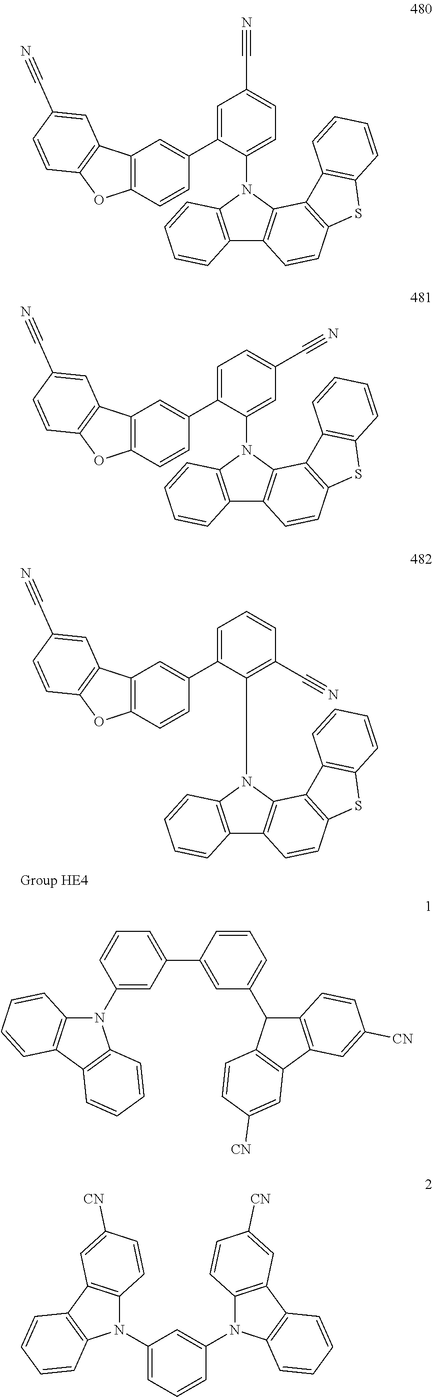

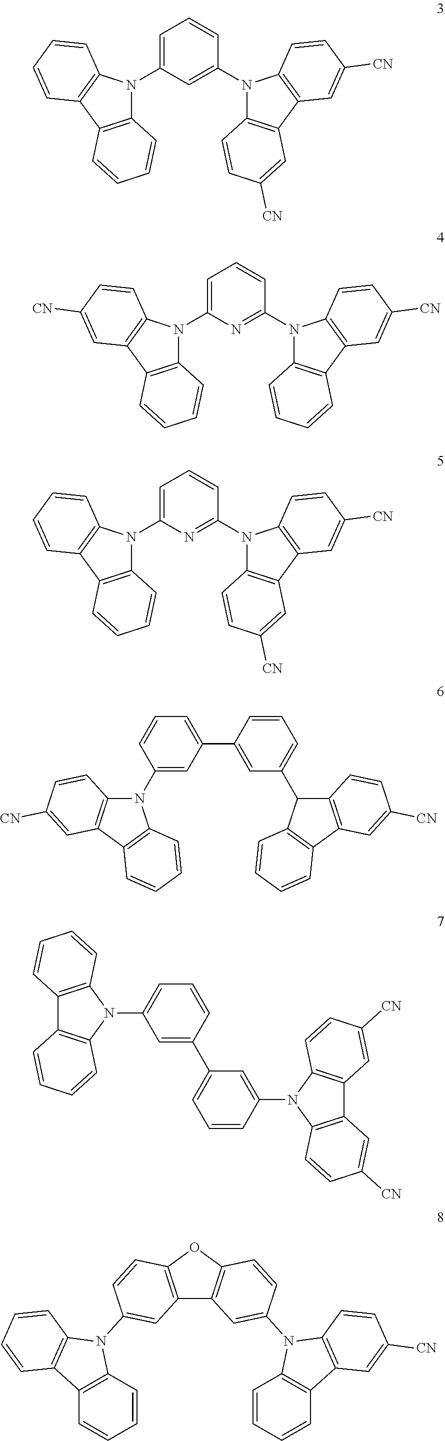

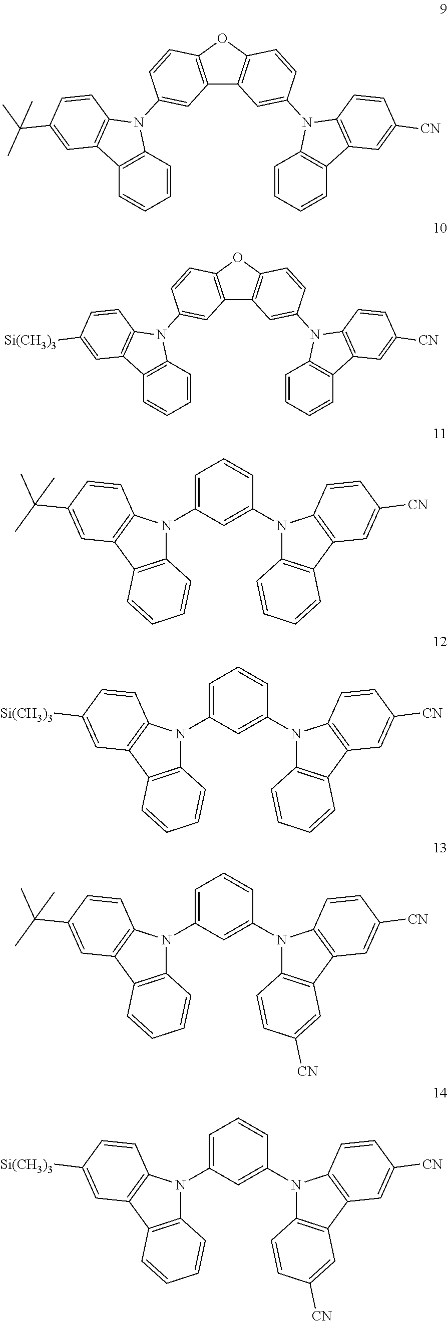

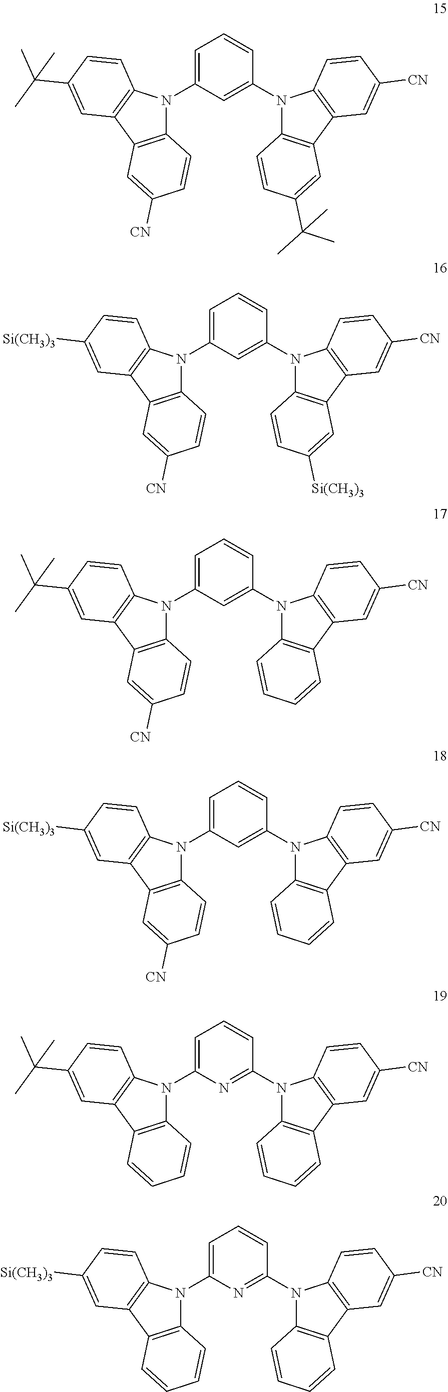

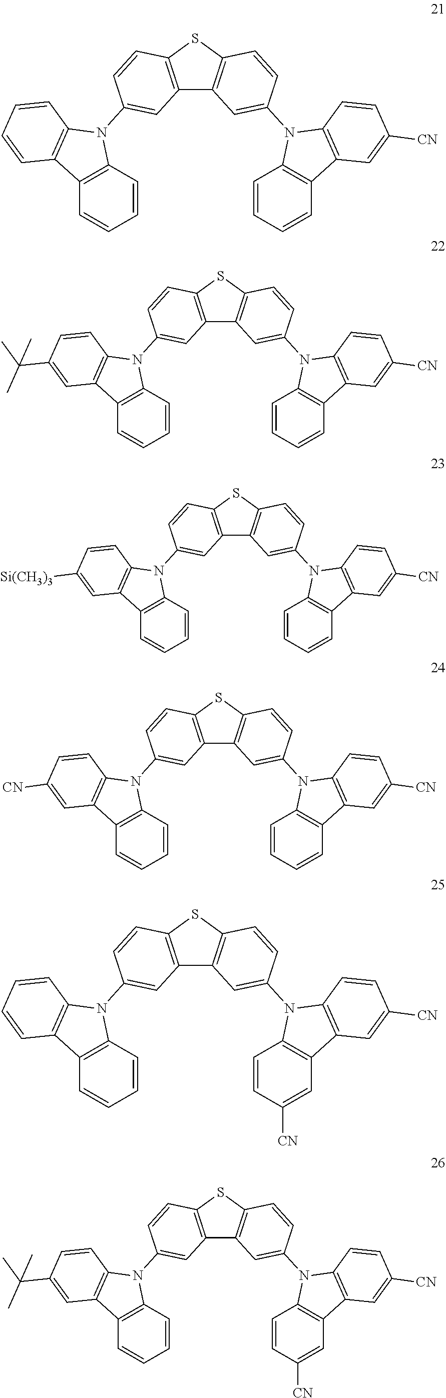

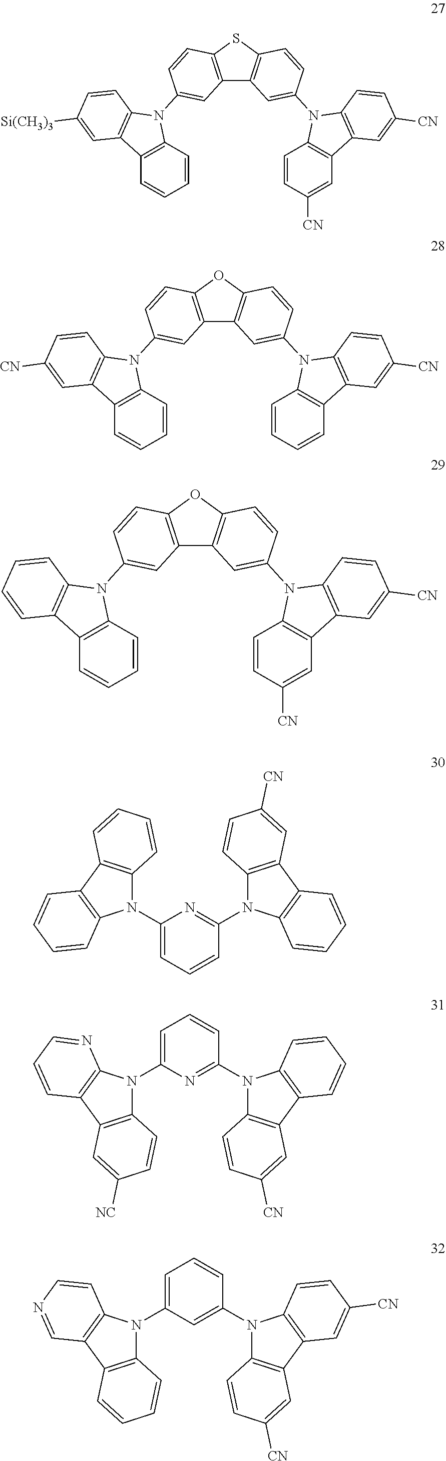

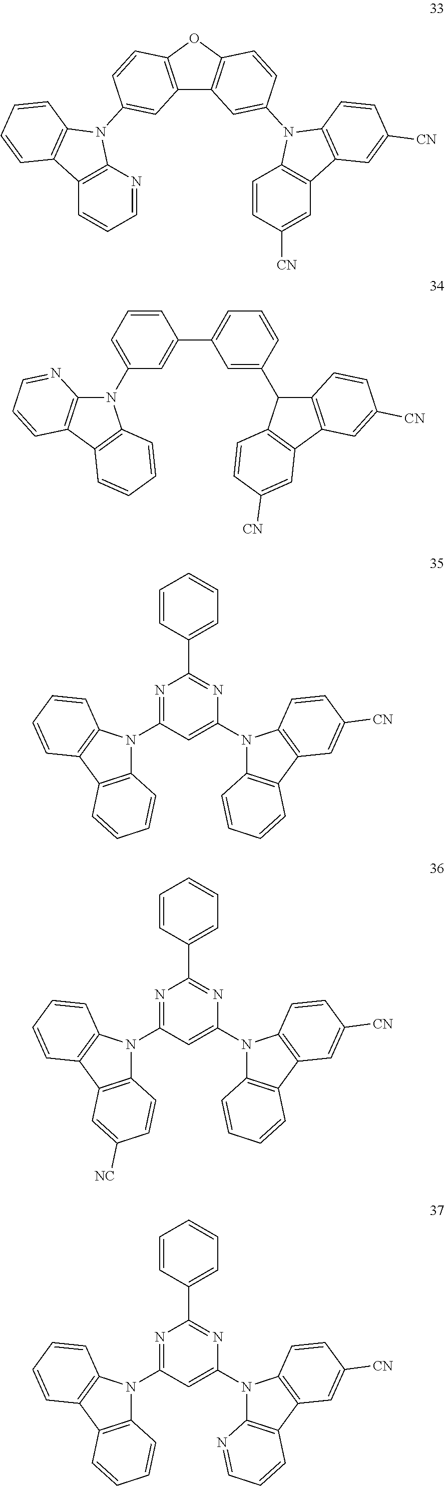

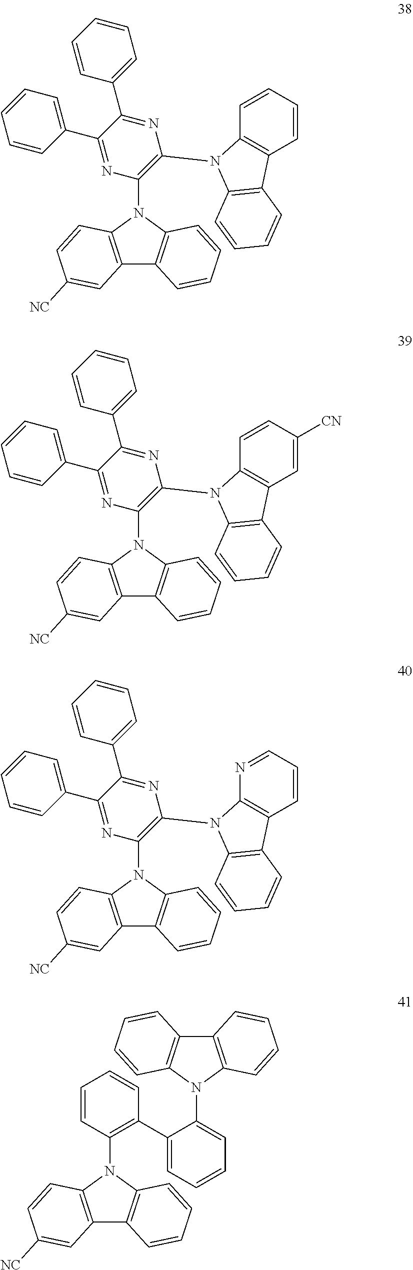 [SMILES: N#Cc1ccc2c(c1)c1ccccc1n2-c1nc(-c2ccccc2)c(-c2ccccc2)nc1-n1c2ccccc2c2ccccc21 ; N#Cc1ccc2c(c1)c1ccccc1n2-c1nc(-c2ccccc2)c(-c2ccccc2)nc1-n1c2ccccc2c2cc(C#N)ccc21 ; N#Cc1ccc2c(c1)c1ccccc1n2-c1nc(-c2ccccc2)c(-c2ccccc2)nc1-n1c2ccccc2c2cccnc21 ; N#Cc1ccc2c(c1)c1ccccc1n2-c1ccccc1-c1ccccc1-n1c2ccccc2c2ccccc21]

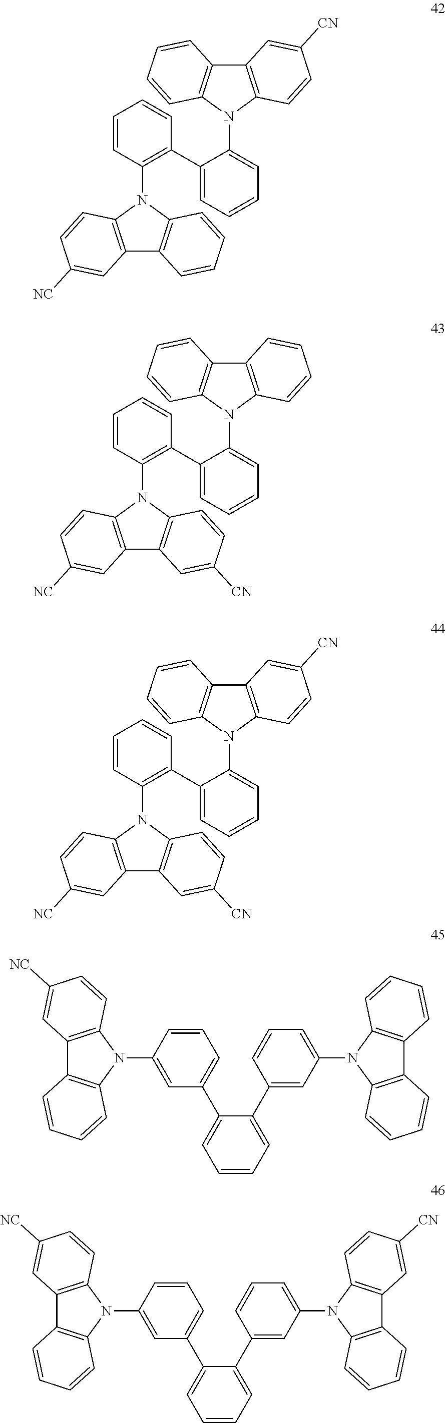 [SMILES: N#Cc1ccc2c(c1)c1ccccc1n2-c1ccccc1-c1ccccc1-n1c2ccccc2c2cc(C#N)ccc21 ; N#Cc1ccc2c(c1)c1cc(C#N)ccc1n2-c1ccccc1-c1ccccc1-n1c2ccccc2c2ccccc21 ; N#Cc1ccc2c(c1)c1ccccc1n2-c1ccccc1-c1ccccc1-n1c2ccc(C#N)cc2c2cc(C#N)ccc21 ; N#Cc1ccc2c(c1)c1ccccc1n2-c1cccc(-c2ccccc2-c2cccc(-n3c4ccccc4c4ccccc43)c2)c1 ; N#Cc1ccc2c(c1)c1ccccc1n2-c1cccc(-c2ccccc2-c2cccc(-n3c4ccccc4c4cc(C#N)ccc43)c2)c1]

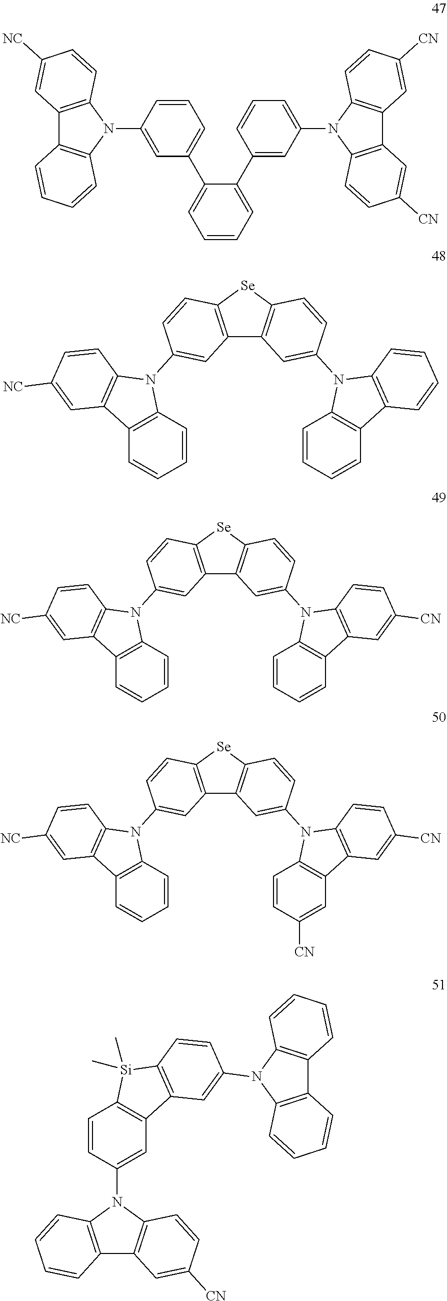

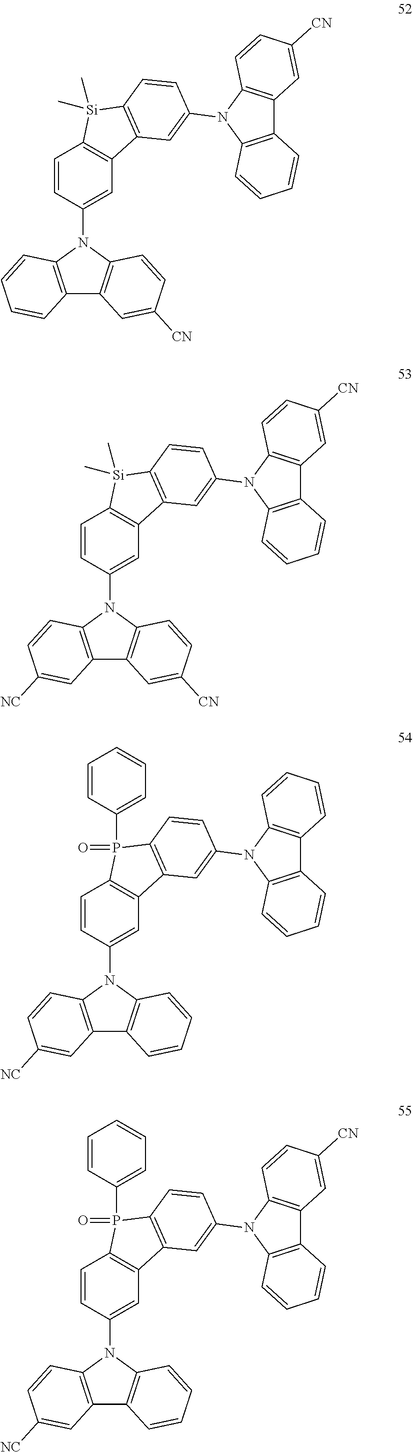 [SMILES: C[Si]1(C)c2ccc(-n3c4ccccc4c4cc(C#N)ccc43)cc2-c2cc(-n3c4ccccc4c4cc(C#N)ccc43)ccc21 ; C[Si]1(C)c2ccc(-n3c4ccccc4c4cc(C#N)ccc43)cc2-c2cc(-n3c4ccc(C#N)cc4c4cc(C#N)ccc43)ccc21 ; N#Cc1ccc2c(c1)c1ccccc1n2-c1ccc2c(c1)-c1cc(-n3c4ccccc4c4ccccc43)ccc1P2(=O)c1ccccc1 ; N#Cc1ccc2c(c1)c1ccccc1n2-c1ccc2c(c1)-c1cc(-n3c4ccccc4c4cc(C#N)ccc43)ccc1P2(=O)c1ccccc1]

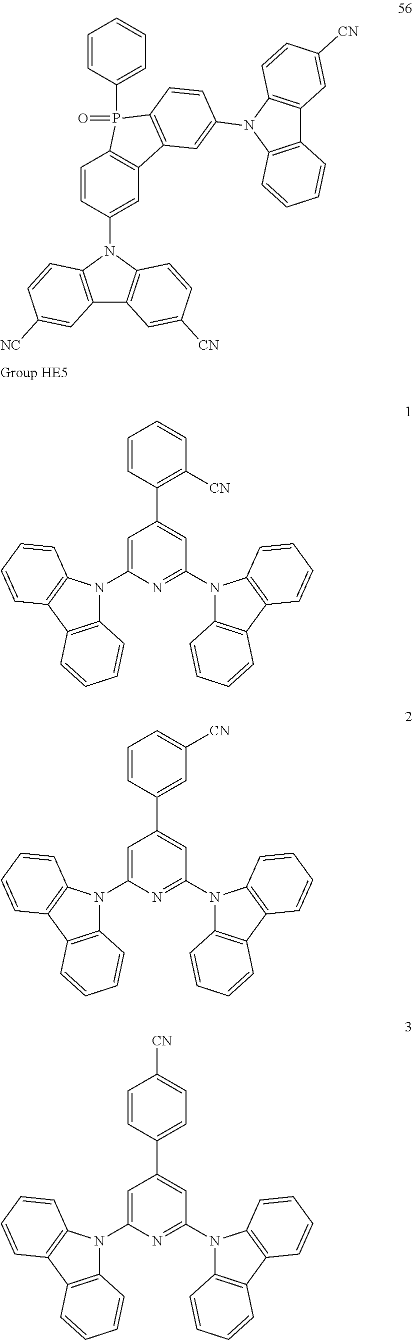

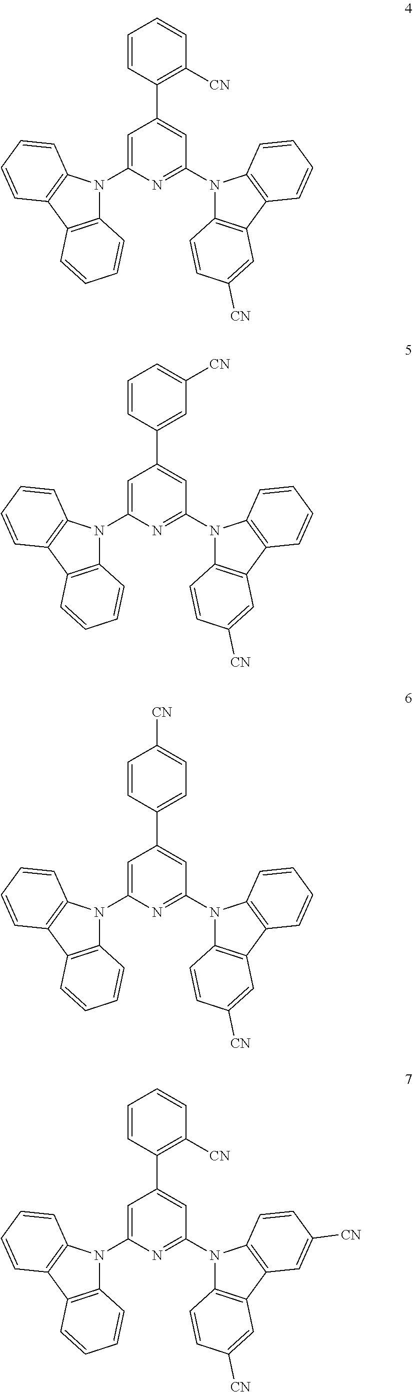 [SMILES: N#Cc1ccc2c(c1)c1ccccc1n2-c1cc(-c2ccccc2C#N)cc(-n2c3ccccc3c3ccccc32)n1 ; N#Cc1cccc(-c2cc(-n3c4ccccc4c4ccccc43)nc(-n3c4ccccc4c4cc(C#N)ccc43)c2)c1 ; N#Cc1ccc(-c2cc(-n3c4ccccc4c4ccccc43)nc(-n3c4ccccc4c4cc(C#N)ccc43)c2)cc1 ; N#Cc1ccc2c(c1)c1cc(C#N)ccc1n2-c1cc(-c2ccccc2C#N)cc(-n2c3ccccc3c3ccccc32)n1]

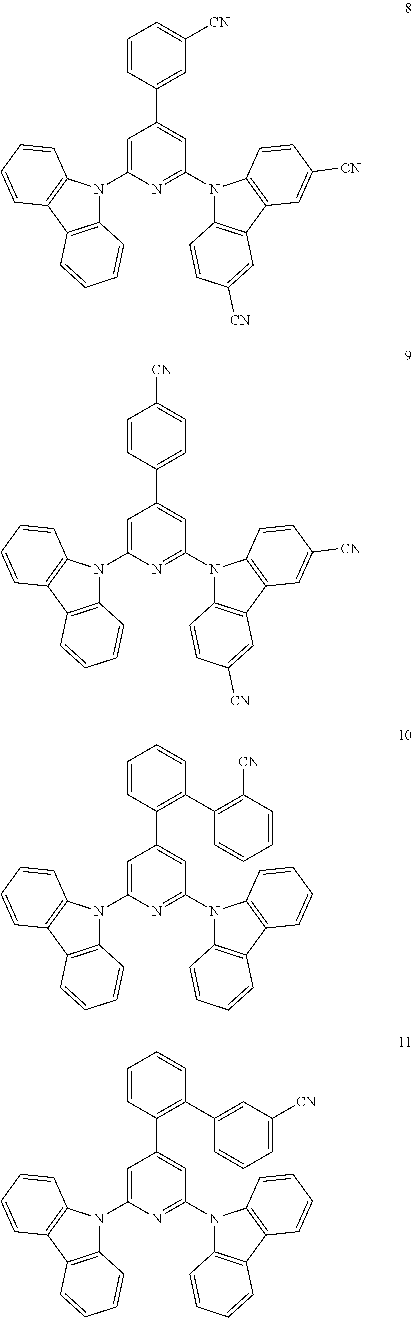 [SMILES: N#Cc1cccc(-c2cc(-n3c4ccccc4c4ccccc43)nc(-n3c4ccc(C#N)cc4c4cc(C#N)ccc43)c2)c1 ; N#Cc1ccc(-c2cc(-n3c4ccccc4c4ccccc43)nc(-n3c4ccc(C#N)cc4c4cc(C#N)ccc43)c2)cc1 ; N#Cc1ccccc1-c1ccccc1-c1cc(-n2c3ccccc3c3ccccc32)nc(-n2c3ccccc3c3ccccc32)c1 ; N#Cc1cccc(-c2ccccc2-c2cc(-n3c4ccccc4c4ccccc43)nc(-n3c4ccccc4c4ccccc43)c2)c1]

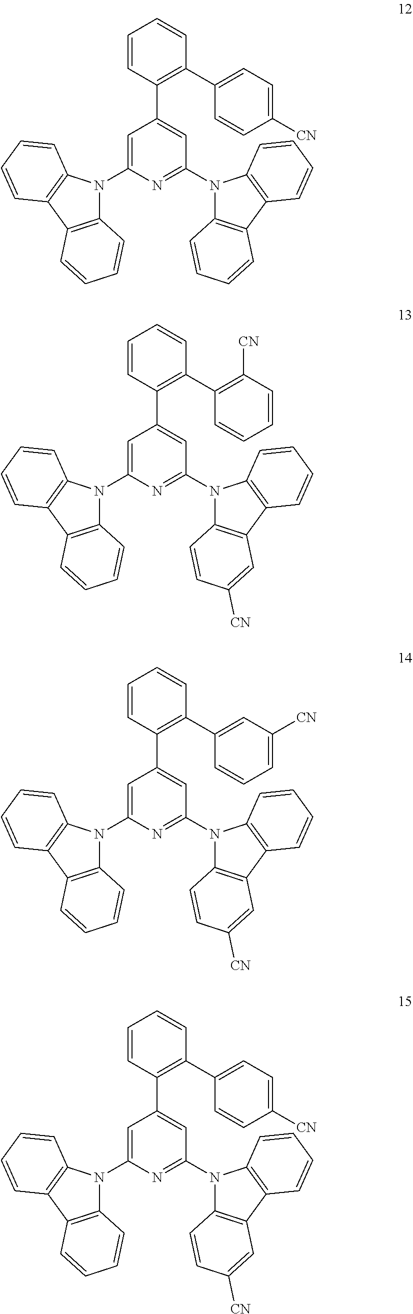 [SMILES: N#Cc1ccc(-c2ccccc2-c2cc(-n3c4ccccc4c4ccccc43)nc(-n3c4ccccc4c4ccccc43)c2)cc1 ; N#Cc1ccc2c(c1)c1ccccc1n2-c1cc(-c2ccccc2-c2ccccc2C#N)cc(-n2c3ccccc3c3ccccc32)n1 ; N#Cc1cccc(-c2ccccc2-c2cc(-n3c4ccccc4c4ccccc43)nc(-n3c4ccccc4c4cc(C#N)ccc43)c2)c1 ; N#Cc1ccc(-c2ccccc2-c2cc(-n3c4ccccc4c4ccccc43)nc(-n3c4ccccc4c4cc(C#N)ccc43)c2)cc1]

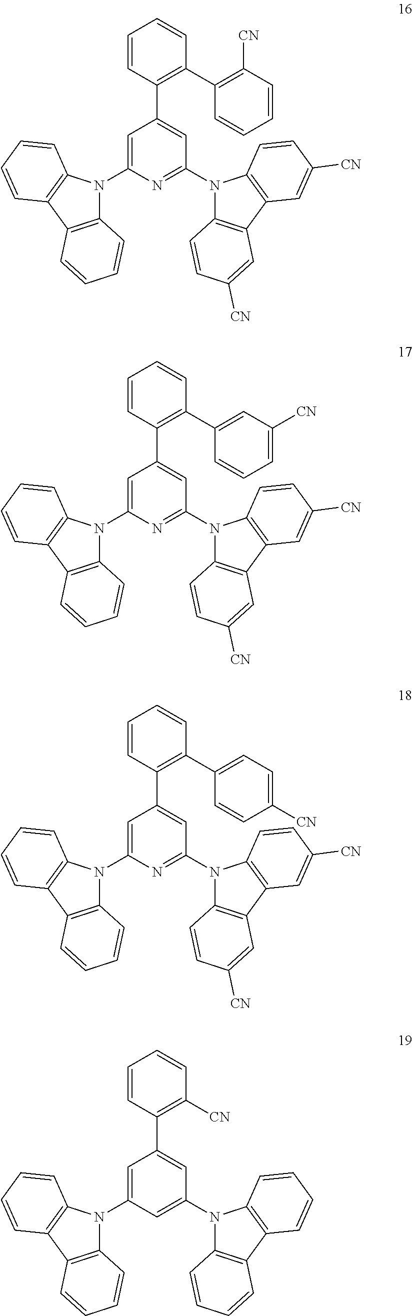 [SMILES: N#Cc1ccc2c(c1)c1cc(C#N)ccc1n2-c1cc(-c2ccccc2-c2ccccc2C#N)cc(-n2c3ccccc3c3ccccc32)n1 ; N#Cc1cccc(-c2ccccc2-c2cc(-n3c4ccccc4c4ccccc43)nc(-n3c4ccc(C#N)cc4c4cc(C#N)ccc43)c2)c1 ; N#Cc1ccc(-c2ccccc2-c2cc(-n3c4ccccc4c4ccccc43)nc(-n3c4ccc(C#N)cc4c4cc(C#N)ccc43)c2)cc1 ; N#Cc1ccccc1-c1cc(-n2c3ccccc3c3ccccc32)cc(-n2c3ccccc3c3ccccc32)c1]

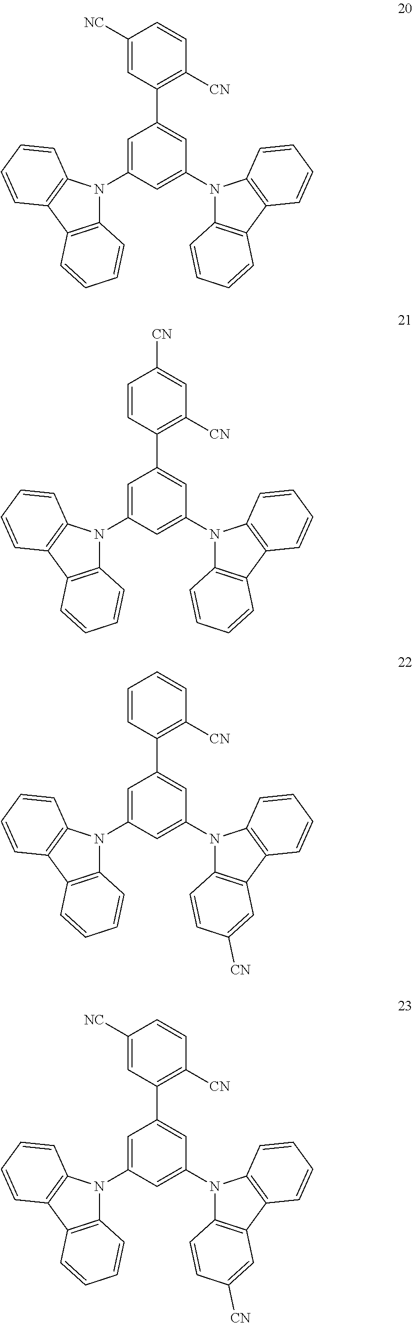 [SMILES: N#Cc1ccc(C#N)c(-c2cc(-n3c4ccccc4c4ccccc43)cc(-n3c4ccccc4c4ccccc43)c2)c1 ; N#Cc1ccc(-c2cc(-n3c4ccccc4c4ccccc43)cc(-n3c4ccccc4c4ccccc43)c2)c(C#N)c1 ; N#Cc1ccc2c(c1)c1ccccc1n2-c1cc(-c2ccccc2C#N)cc(-n2c3ccccc3c3ccccc32)c1 ; N#Cc1ccc(C#N)c(-c2cc(-n3c4ccccc4c4ccccc43)cc(-n3c4ccccc4c4cc(C#N)ccc43)c2)c1]

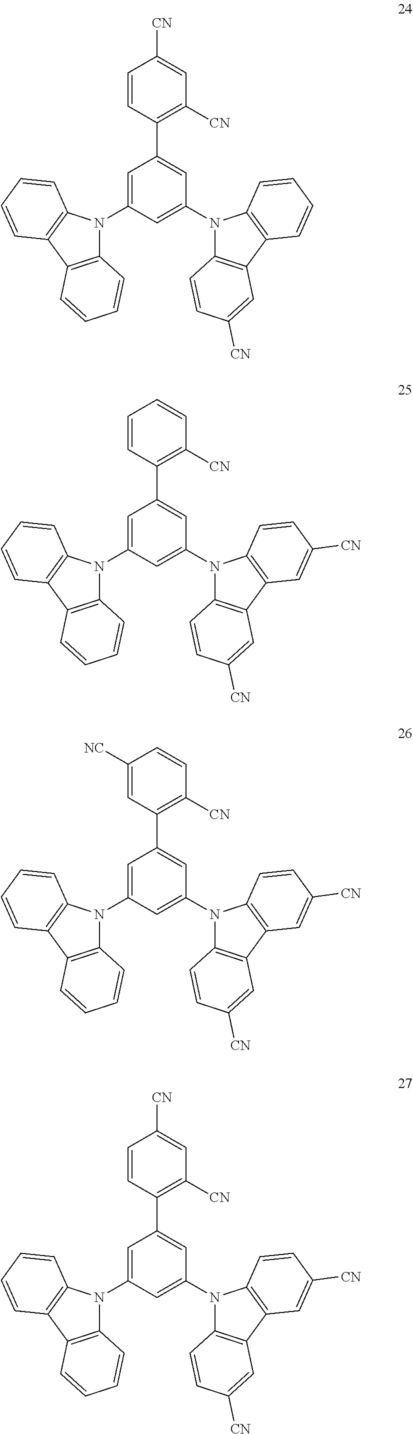 [SMILES: N#Cc1ccc(-c2cc(-n3c4ccccc4c4ccccc43)cc(-n3c4ccccc4c4cc(C#N)ccc43)c2)c(C#N)c1 ; N#Cc1ccc2c(c1)c1cc(C#N)ccc1n2-c1cc(-c2ccccc2C#N)cc(-n2c3ccccc3c3ccccc32)c1 ; N#Cc1ccc(C#N)c(-c2cc(-n3c4ccccc4c4ccccc43)cc(-n3c4ccc(C#N)cc4c4cc(C#N)ccc43)c2)c1 ; N#Cc1ccc(-c2cc(-n3c4ccccc4c4ccccc43)cc(-n3c4ccc(C#N)cc4c4cc(C#N)ccc43)c2)c(C#N)c1]

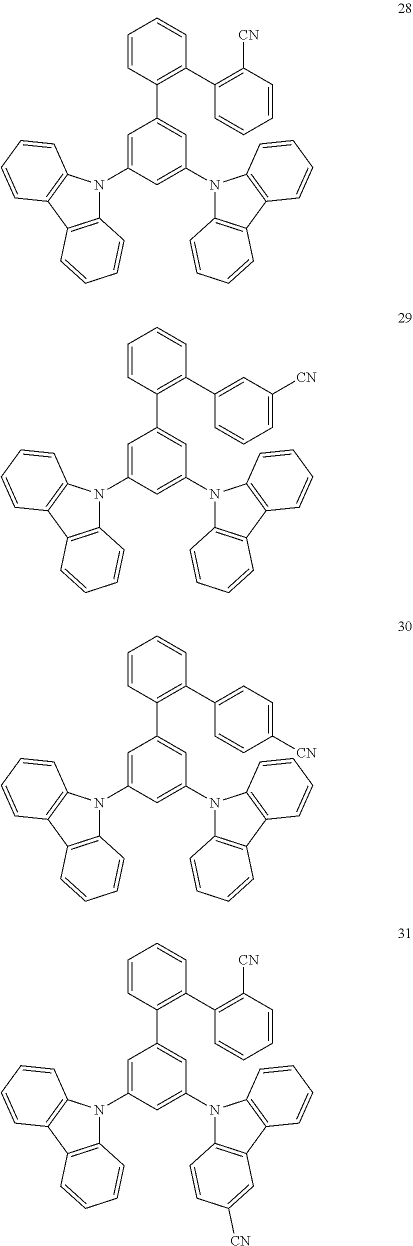 [SMILES: N#Cc1ccccc1-c1ccccc1-c1cc(-n2c3ccccc3c3ccccc32)cc(-n2c3ccccc3c3ccccc32)c1 ; N#Cc1cccc(-c2ccccc2-c2cc(-n3c4ccccc4c4ccccc43)cc(-n3c4ccccc4c4ccccc43)c2)c1 ; N#Cc1ccc(-c2ccccc2-c2cc(-n3c4ccccc4c4ccccc43)cc(-n3c4ccccc4c4ccccc43)c2)cc1 ; N#Cc1ccc2c(c1)c1ccccc1n2-c1cc(-c2ccccc2-c2ccccc2C#N)cc(-n2c3ccccc3c3ccccc32)c1]

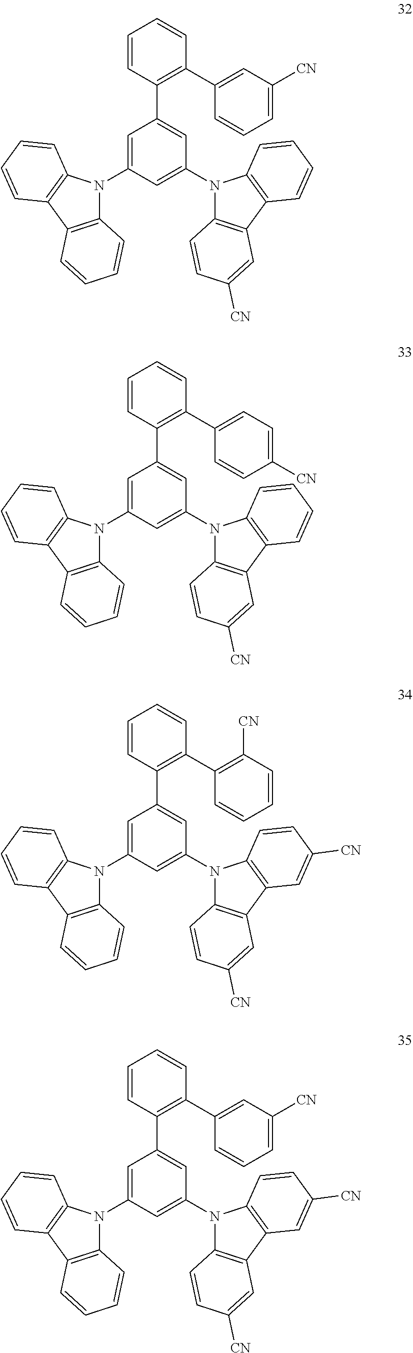 [SMILES: N#Cc1cccc(-c2ccccc2-c2cc(-n3c4ccccc4c4ccccc43)cc(-n3c4ccccc4c4cc(C#N)ccc43)c2)c1 ; N#Cc1ccc(-c2ccccc2-c2cc(-n3c4ccccc4c4ccccc43)cc(-n3c4ccccc4c4cc(C#N)ccc43)c2)cc1 ; N#Cc1ccc2c(c1)c1cc(C#N)ccc1n2-c1cc(-c2ccccc2-c2ccccc2C#N)cc(-n2c3ccccc3c3ccccc32)c1 ; N#Cc1cccc(-c2ccccc2-c2cc(-n3c4ccccc4c4ccccc43)cc(-n3c4ccc(C#N)cc4c4cc(C#N)ccc43)c2)c1]

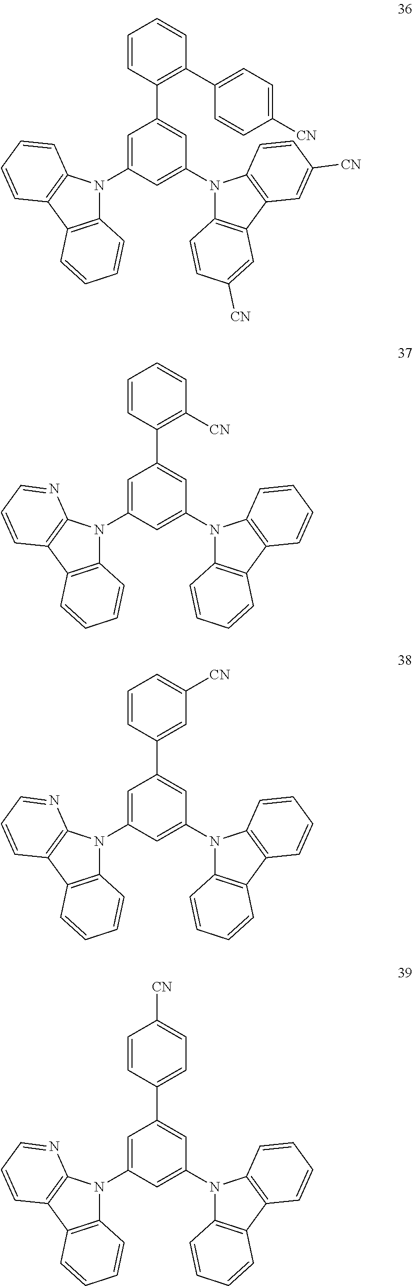 [SMILES: N#Cc1ccc(-c2ccccc2-c2cc(-n3c4ccccc4c4ccccc43)cc(-n3c4ccc(C#N)cc4c4cc(C#N)ccc43)c2)cc1 ; N#Cc1ccccc1-c1cc(-n2c3ccccc3c3ccccc32)cc(-n2c3ccccc3c3cccnc32)c1 ; N#Cc1cccc(-c2cc(-n3c4ccccc4c4ccccc43)cc(-n3c4ccccc4c4cccnc43)c2)c1 ; N#Cc1ccc(-c2cc(-n3c4ccccc4c4ccccc43)cc(-n3c4ccccc4c4cccnc43)c2)cc1]

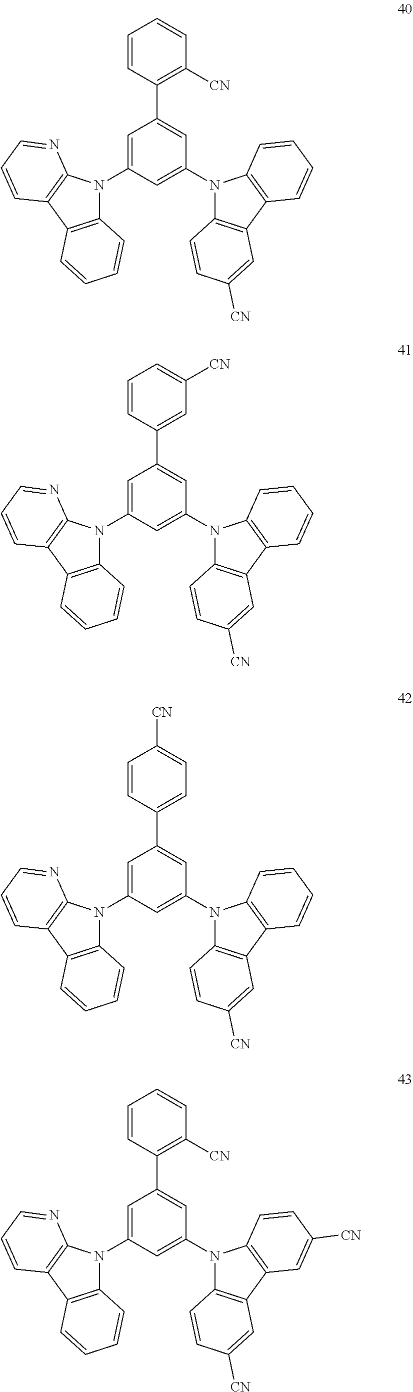 [SMILES: N#Cc1ccc2c(c1)c1ccccc1n2-c1cc(-c2ccccc2C#N)cc(-n2c3ccccc3c3cccnc32)c1 ; N#Cc1cccc(-c2cc(-n3c4ccccc4c4cc(C#N)ccc43)cc(-n3c4ccccc4c4cccnc43)c2)c1 ; N#Cc1ccc(-c2cc(-n3c4ccccc4c4cc(C#N)ccc43)cc(-n3c4ccccc4c4cccnc43)c2)cc1 ; N#Cc1ccc2c(c1)c1cc(C#N)ccc1n2-c1cc(-c2ccccc2C#N)cc(-n2c3ccccc3c3cccnc32)c1]

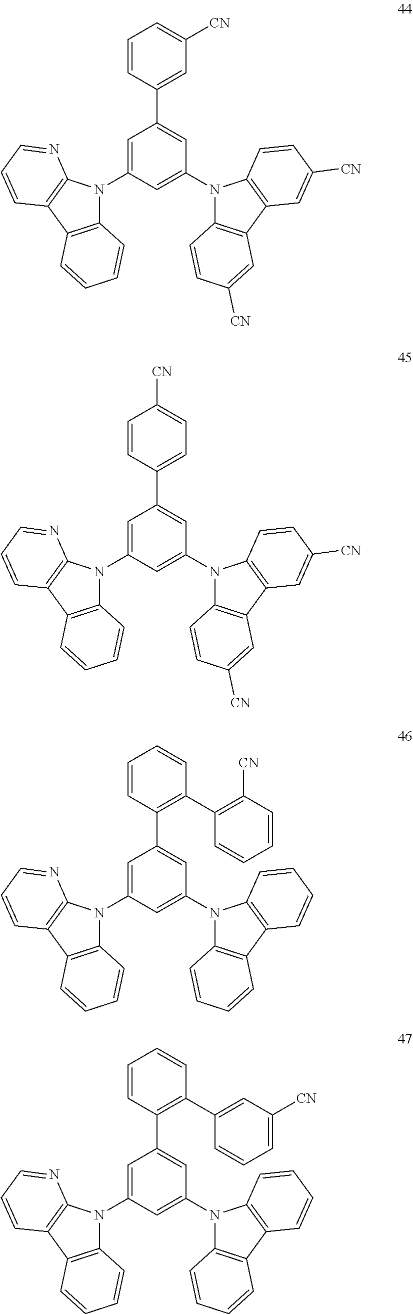 [SMILES: N#Cc1cccc(-c2cc(-n3c4ccc(C#N)cc4c4cc(C#N)ccc43)cc(-n3c4ccccc4c4cccnc43)c2)c1 ; N#Cc1ccc(-c2cc(-n3c4ccc(C#N)cc4c4cc(C#N)ccc43)cc(-n3c4ccccc4c4cccnc43)c2)cc1 ; N#Cc1ccccc1-c1ccccc1-c1cc(-n2c3ccccc3c3ccccc32)cc(-n2c3ccccc3c3cccnc32)c1 ; N#Cc1cccc(-c2ccccc2-c2cc(-n3c4ccccc4c4ccccc43)cc(-n3c4ccccc4c4cccnc43)c2)c1]

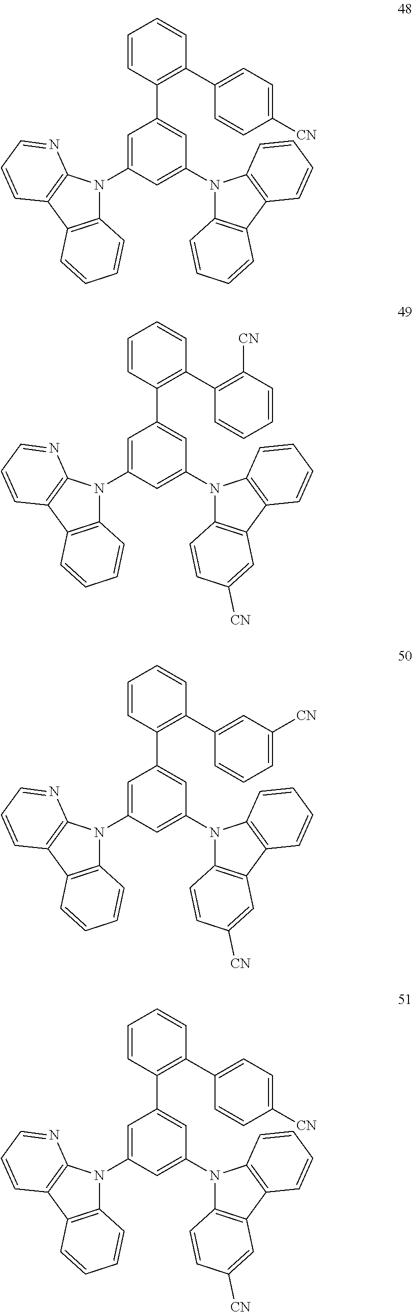 [SMILES: N#Cc1ccc(-c2ccccc2-c2cc(-n3c4ccccc4c4ccccc43)cc(-n3c4ccccc4c4cccnc43)c2)cc1 ; N#Cc1ccc2c(c1)c1ccccc1n2-c1cc(-c2ccccc2-c2ccccc2C#N)cc(-n2c3ccccc3c3cccnc32)c1 ; N#Cc1cccc(-c2ccccc2-c2cc(-n3c4ccccc4c4cc(C#N)ccc43)cc(-n3c4ccccc4c4cccnc43)c2)c1 ; N#Cc1ccc(-c2ccccc2-c2cc(-n3c4ccccc4c4cc(C#N)ccc43)cc(-n3c4ccccc4c4cccnc43)c2)cc1]

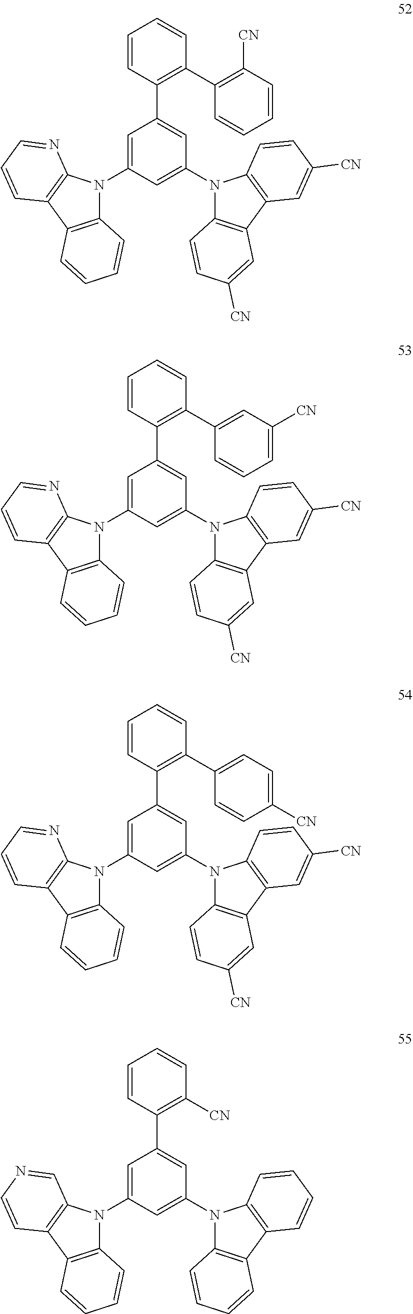 [SMILES: N#Cc1ccc2c(c1)c1cc(C#N)ccc1n2-c1cc(-c2ccccc2-c2ccccc2C#N)cc(-n2c3ccccc3c3cccnc32)c1 ; N#Cc1cccc(-c2ccccc2-c2cc(-n3c4ccc(C#N)cc4c4cc(C#N)ccc43)cc(-n3c4ccccc4c4cccnc43)c2)c1 ; N#Cc1ccc(-c2ccccc2-c2cc(-n3c4ccc(C#N)cc4c4cc(C#N)ccc43)cc(-n3c4ccccc4c4cccnc43)c2)cc1 ; N#Cc1ccccc1-c1cc(-n2c3ccccc3c3ccccc32)cc(-n2c3ccccc3c3ccncc32)c1]

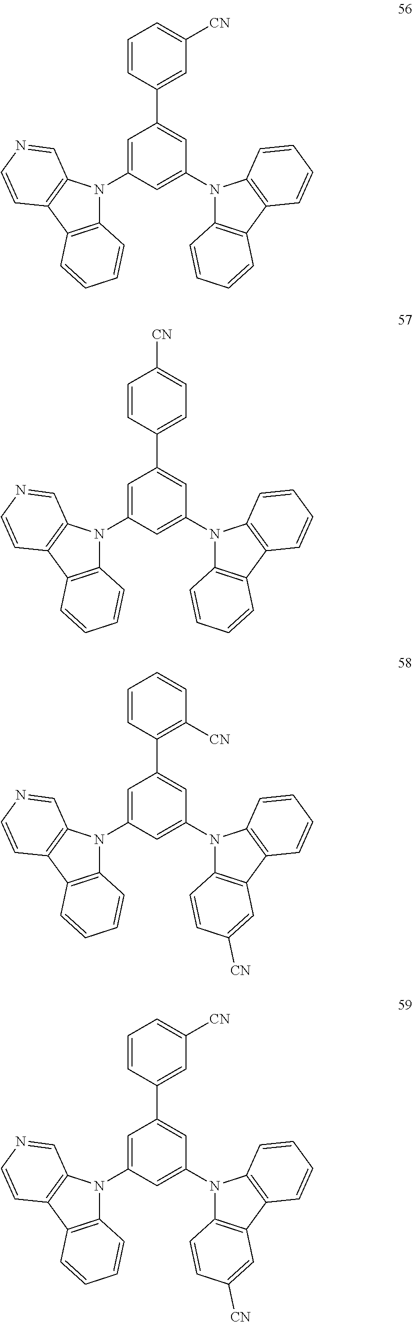 [SMILES: N#Cc1cccc(-c2cc(-n3c4ccccc4c4ccccc43)cc(-n3c4ccccc4c4ccncc43)c2)c1 ; N#Cc1ccc(-c2cc(-n3c4ccccc4c4ccccc43)cc(-n3c4ccccc4c4ccncc43)c2)cc1 ; N#Cc1ccc2c(c1)c1ccccc1n2-c1cc(-c2ccccc2C#N)cc(-n2c3ccccc3c3ccncc32)c1 ; N#Cc1cccc(-c2cc(-n3c4ccccc4c4cc(C#N)ccc43)cc(-n3c4ccccc4c4ccncc43)c2)c1]

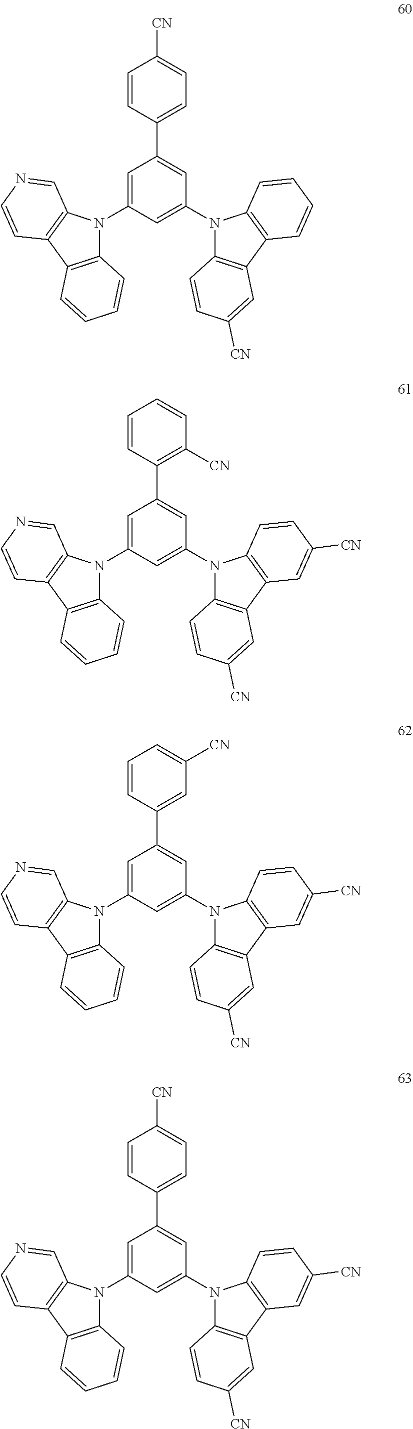 [SMILES: N#Cc1ccc(-c2cc(-n3c4ccccc4c4cc(C#N)ccc43)cc(-n3c4ccccc4c4ccncc43)c2)cc1 ; N#Cc1ccc2c(c1)c1cc(C#N)ccc1n2-c1cc(-c2ccccc2C#N)cc(-n2c3ccccc3c3ccncc32)c1 ; N#Cc1cccc(-c2cc(-n3c4ccc(C#N)cc4c4cc(C#N)ccc43)cc(-n3c4ccccc4c4ccncc43)c2)c1 ; N#Cc1ccc(-c2cc(-n3c4ccc(C#N)cc4c4cc(C#N)ccc43)cc(-n3c4ccccc4c4ccncc43)c2)cc1]

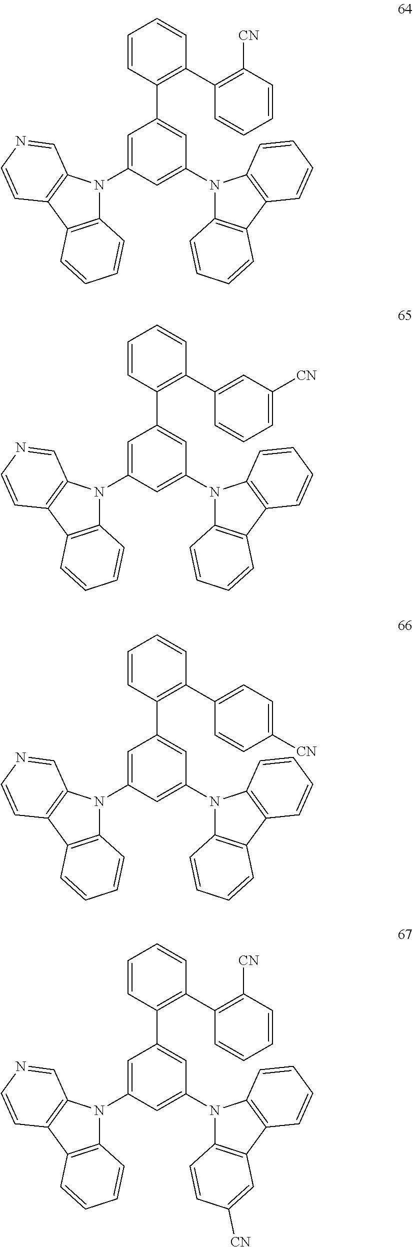 [SMILES: N#Cc1ccccc1-c1ccccc1-c1cc(-n2c3ccccc3c3ccccc32)cc(-n2c3ccccc3c3ccncc32)c1 ; N#Cc1cccc(-c2ccccc2-c2cc(-n3c4ccccc4c4ccccc43)cc(-n3c4ccccc4c4ccncc43)c2)c1 ; N#Cc1ccc(-c2ccccc2-c2cc(-n3c4ccccc4c4ccccc43)cc(-n3c4ccccc4c4ccncc43)c2)cc1 ; N#Cc1ccc2c(c1)c1ccccc1n2-c1cc(-c2ccccc2-c2ccccc2C#N)cc(-n2c3ccccc3c3ccncc32)c1]

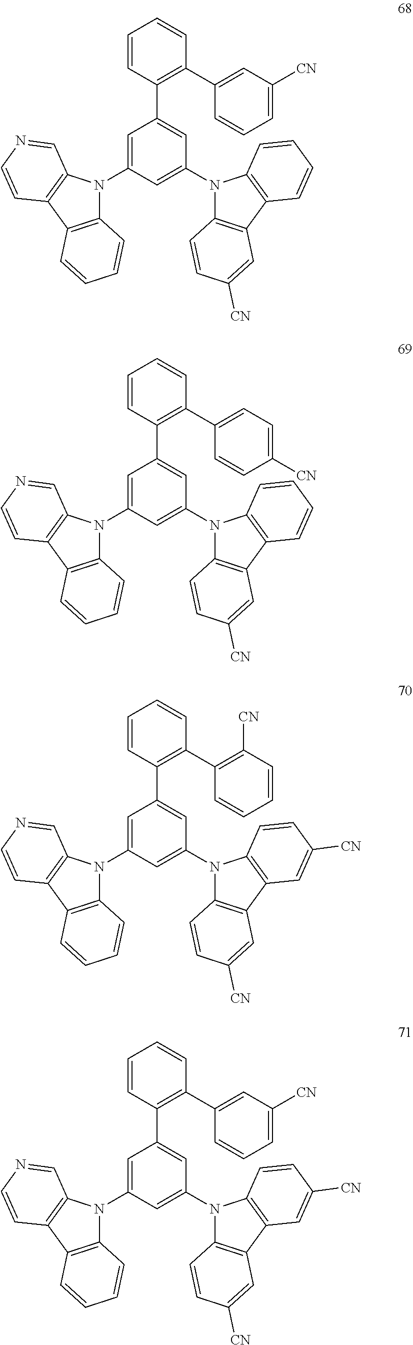 [SMILES: N#Cc1cccc(-c2ccccc2-c2cc(-n3c4ccccc4c4cc(C#N)ccc43)cc(-n3c4ccccc4c4ccncc43)c2)c1 ; N#Cc1ccc(-c2ccccc2-c2cc(-n3c4ccccc4c4cc(C#N)ccc43)cc(-n3c4ccccc4c4ccncc43)c2)cc1 ; N#Cc1ccc2c(c1)c1cc(C#N)ccc1n2-c1cc(-c2ccccc2-c2ccccc2C#N)cc(-n2c3ccccc3c3ccncc32)c1 ; N#Cc1cccc(-c2ccccc2-c2cc(-n3c4ccc(C#N)cc4c4cc(C#N)ccc43)cc(-n3c4ccccc4c4ccncc43)c2)c1]

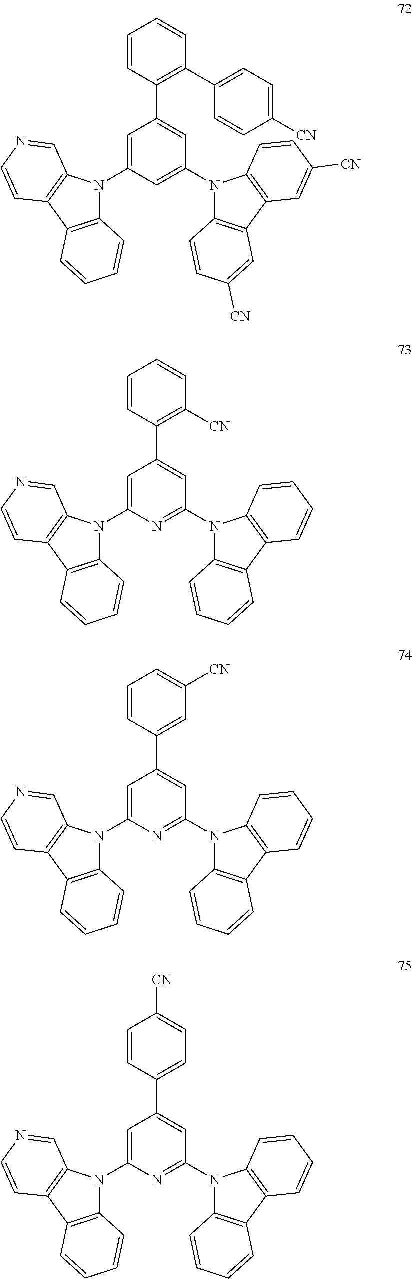

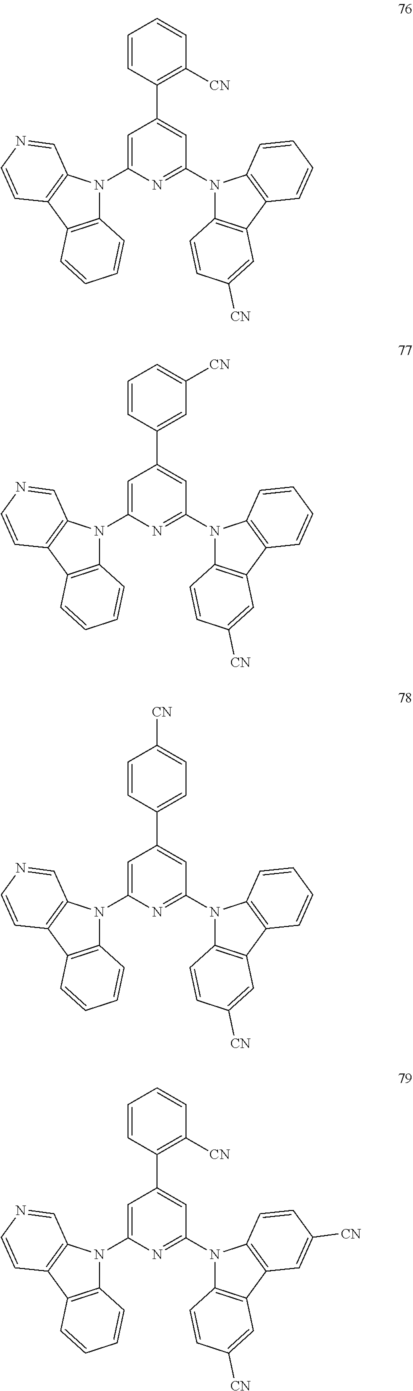 [SMILES: N#Cc1ccc2c(c1)c1ccccc1n2-c1cc(-c2ccccc2C#N)cc(-n2c3ccccc3c3ccncc32)n1 ; N#Cc1cccc(-c2cc(-n3c4ccccc4c4cc(C#N)ccc43)nc(-n3c4ccccc4c4ccncc43)c2)c1 ; N#Cc1ccc(-c2cc(-n3c4ccccc4c4cc(C#N)ccc43)nc(-n3c4ccccc4c4ccncc43)c2)cc1 ; N#Cc1ccc2c(c1)c1cc(C#N)ccc1n2-c1cc(-c2ccccc2C#N)cc(-n2c3ccccc3c3ccncc32)n1]

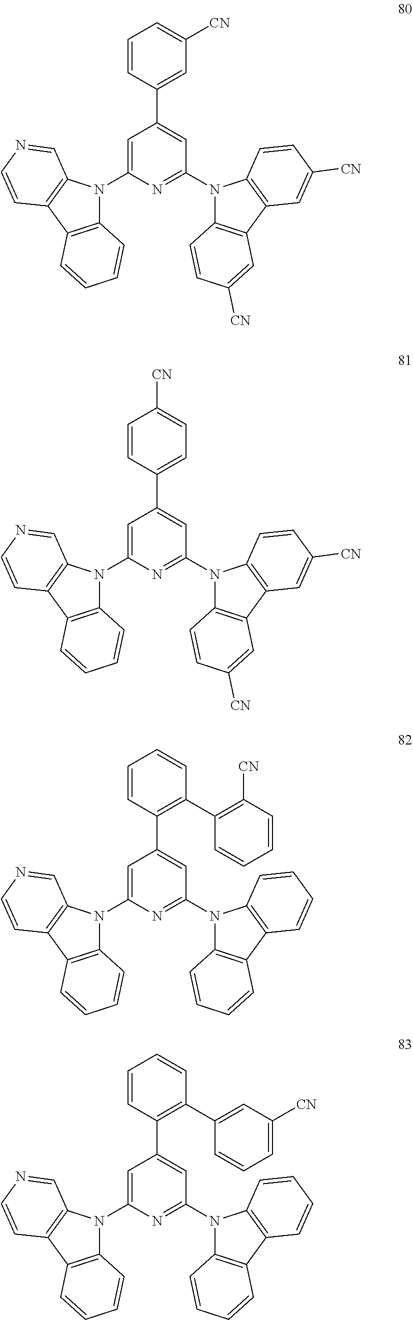 [SMILES: N#Cc1cccc(-c2cc(-n3c4ccc(C#N)cc4c4cc(C#N)ccc43)nc(-n3c4ccccc4c4ccncc43)c2)c1 ; N#Cc1ccc(-c2cc(-n3c4ccc(C#N)cc4c4cc(C#N)ccc43)nc(-n3c4ccccc4c4ccncc43)c2)cc1 ; N#Cc1ccccc1-c1ccccc1-c1cc(-n2c3ccccc3c3ccccc32)nc(-n2c3ccccc3c3ccncc32)c1 ; N#Cc1cccc(-c2ccccc2-c2cc(-n3c4ccccc4c4ccccc43)nc(-n3c4ccccc4c4ccncc43)c2)c1]

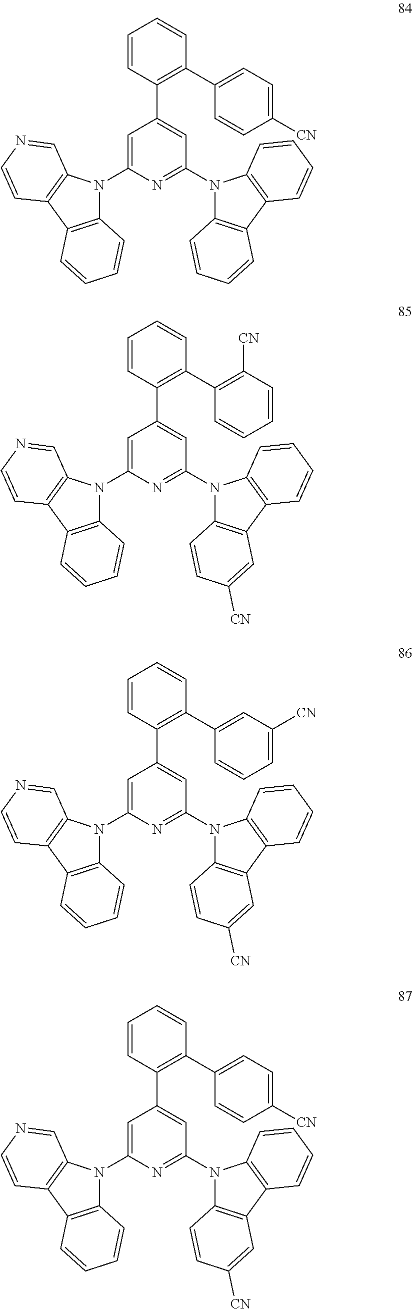 [SMILES: N#Cc1ccc(-c2ccccc2-c2cc(-n3c4ccccc4c4ccccc43)nc(-n3c4ccccc4c4ccncc43)c2)cc1 ; N#Cc1ccc2c(c1)c1ccccc1n2-c1cc(-c2ccccc2-c2ccccc2C#N)cc(-n2c3ccccc3c3ccncc32)n1 ; N#Cc1cccc(-c2ccccc2-c2cc(-n3c4ccccc4c4cc(C#N)ccc43)nc(-n3c4ccccc4c4ccncc43)c2)c1 ; N#Cc1ccc(-c2ccccc2-c2cc(-n3c4ccccc4c4cc(C#N)ccc43)nc(-n3c4ccccc4c4ccncc43)c2)cc1]

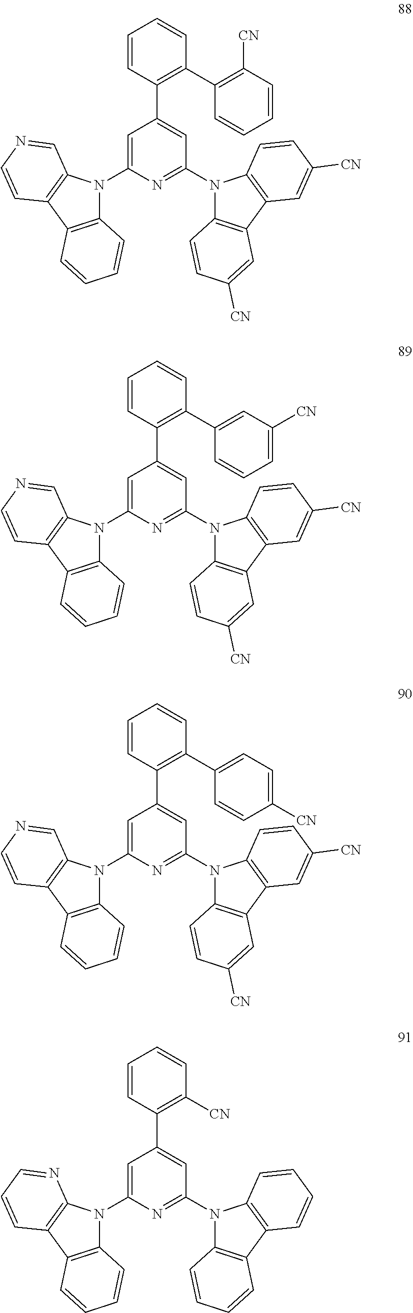 [SMILES: N#Cc1ccc2c(c1)c1cc(C#N)ccc1n2-c1cc(-c2ccccc2-c2ccccc2C#N)cc(-n2c3ccccc3c3ccncc32)n1 ; N#Cc1cccc(-c2ccccc2-c2cc(-n3c4ccc(C#N)cc4c4cc(C#N)ccc43)nc(-n3c4ccccc4c4ccncc43)c2)c1 ; N#Cc1ccc(-c2ccccc2-c2cc(-n3c4ccc(C#N)cc4c4cc(C#N)ccc43)nc(-n3c4ccccc4c4ccncc43)c2)cc1 ; N#Cc1ccccc1-c1cc(-n2c3ccccc3c3ccccc32)nc(-n2c3ccccc3c3cccnc32)c1]

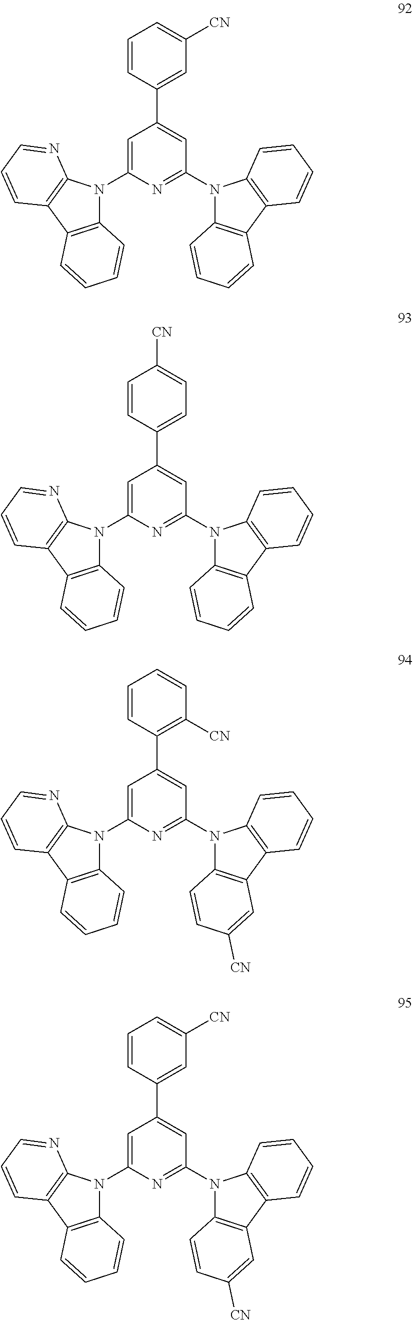 [SMILES: N#Cc1cccc(-c2cc(-n3c4ccccc4c4ccccc43)nc(-n3c4ccccc4c4cccnc43)c2)c1 ; N#Cc1ccc(-c2cc(-n3c4ccccc4c4ccccc43)nc(-n3c4ccccc4c4cccnc43)c2)cc1 ; N#Cc1ccc2c(c1)c1ccccc1n2-c1cc(-c2ccccc2C#N)cc(-n2c3ccccc3c3cccnc32)n1 ; N#Cc1cccc(-c2cc(-n3c4ccccc4c4cc(C#N)ccc43)nc(-n3c4ccccc4c4cccnc43)c2)c1]

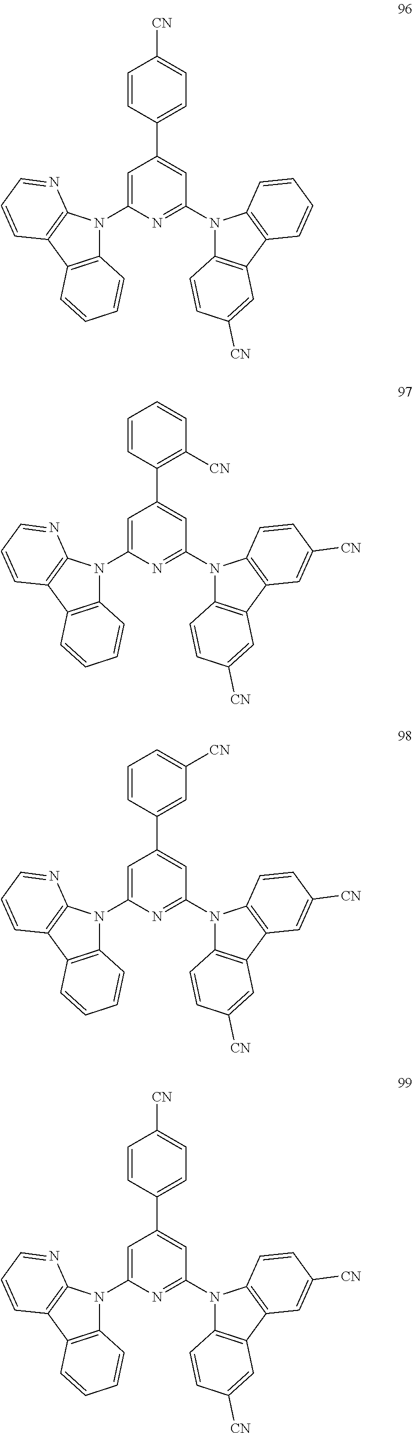 [SMILES: N#Cc1ccc(-c2cc(-n3c4ccccc4c4cc(C#N)ccc43)nc(-n3c4ccccc4c4cccnc43)c2)cc1 ; N#Cc1ccc2c(c1)c1cc(C#N)ccc1n2-c1cc(-c2ccccc2C#N)cc(-n2c3ccccc3c3cccnc32)n1 ; N#Cc1cccc(-c2cc(-n3c4ccc(C#N)cc4c4cc(C#N)ccc43)nc(-n3c4ccccc4c4cccnc43)c2)c1 ; N#Cc1ccc(-c2cc(-n3c4ccc(C#N)cc4c4cc(C#N)ccc43)nc(-n3c4ccccc4c4cccnc43)c2)cc1]

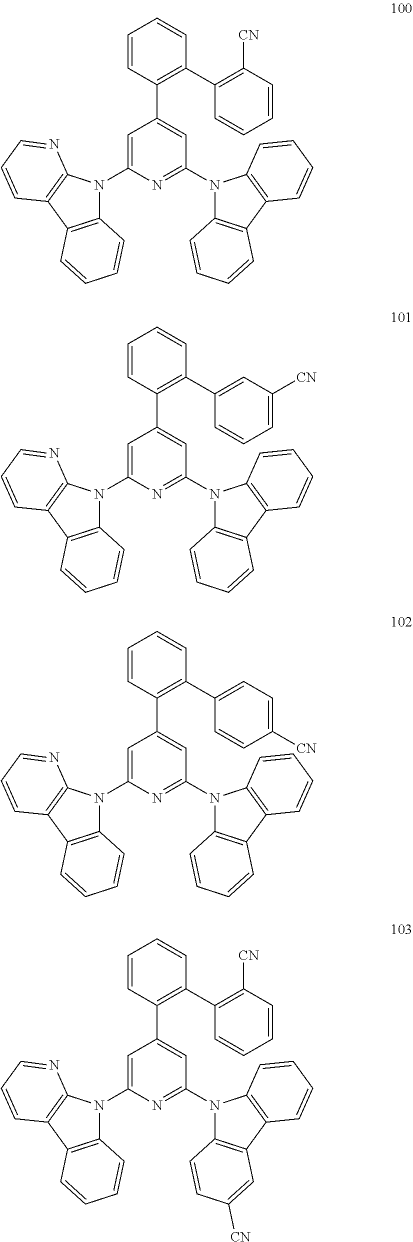 [SMILES: N#Cc1ccccc1-c1ccccc1-c1cc(-n2c3ccccc3c3ccccc32)nc(-n2c3ccccc3c3cccnc32)c1 ; N#Cc1cccc(-c2ccccc2-c2cc(-n3c4ccccc4c4ccccc43)nc(-n3c4ccccc4c4cccnc43)c2)c1 ; N#Cc1ccc(-c2ccccc2-c2cc(-n3c4ccccc4c4ccccc43)nc(-n3c4ccccc4c4cccnc43)c2)cc1 ; N#Cc1ccc2c(c1)c1ccccc1n2-c1cc(-c2ccccc2-c2ccccc2C#N)cc(-n2c3ccccc3c3cccnc32)n1]

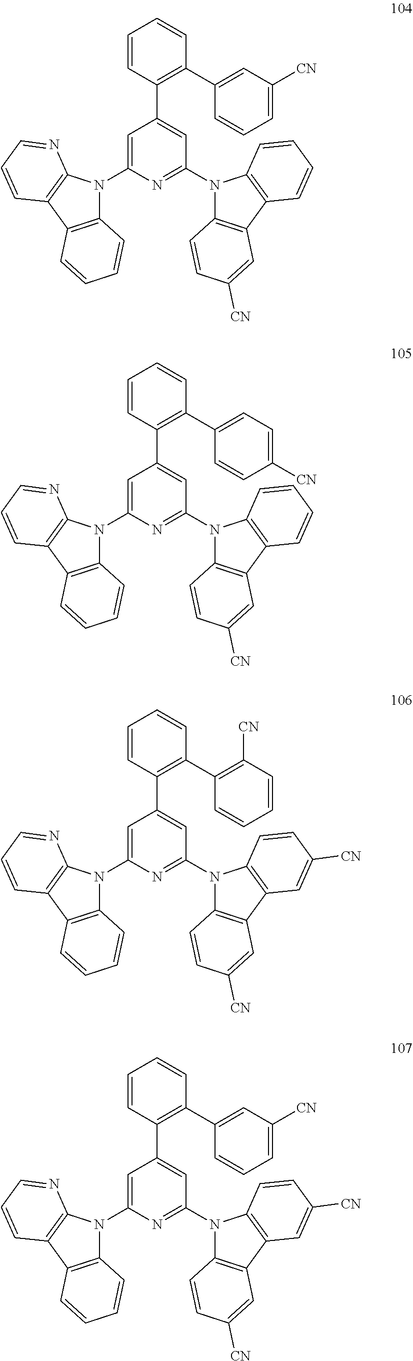 [SMILES: N#Cc1cccc(-c2ccccc2-c2cc(-n3c4ccccc4c4cc(C#N)ccc43)nc(-n3c4ccccc4c4cccnc43)c2)c1 ; N#Cc1ccc(-c2ccccc2-c2cc(-n3c4ccccc4c4cc(C#N)ccc43)nc(-n3c4ccccc4c4cccnc43)c2)cc1 ; N#Cc1ccc2c(c1)c1cc(C#N)ccc1n2-c1cc(-c2ccccc2-c2ccccc2C#N)cc(-n2c3ccccc3c3cccnc32)n1 ; N#Cc1cccc(-c2ccccc2-c2cc(-n3c4ccc(C#N)cc4c4cc(C#N)ccc43)nc(-n3c4ccccc4c4cccnc43)c2)c1]

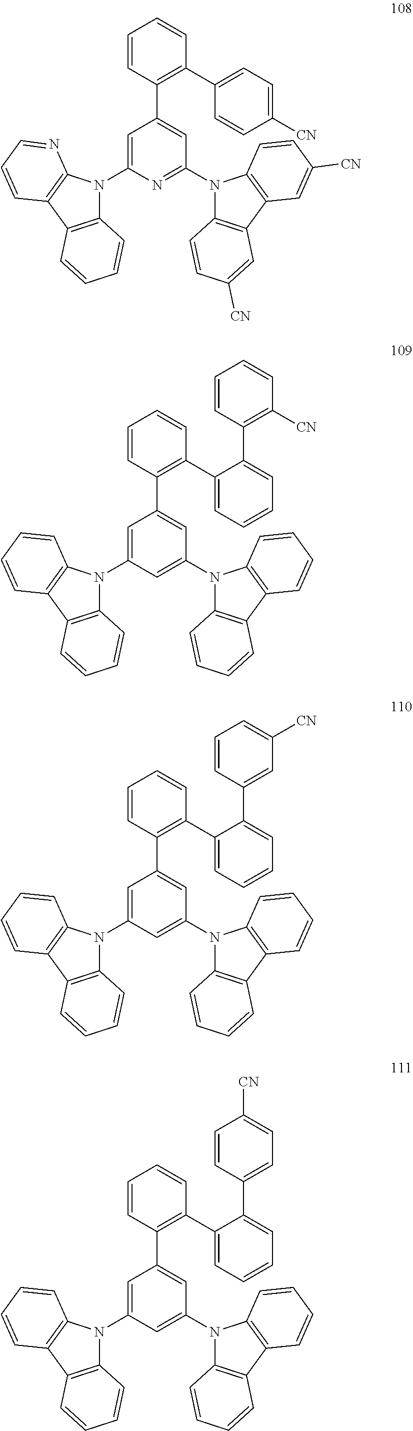

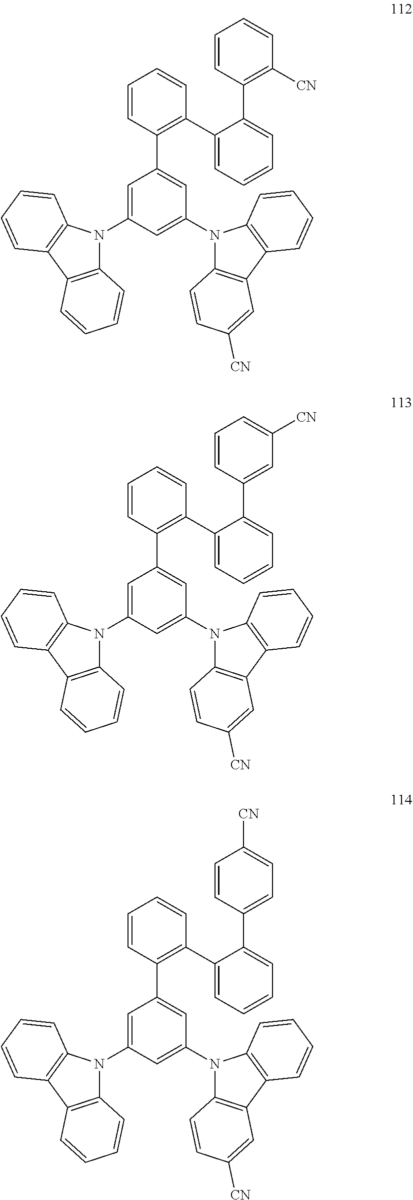 [SMILES: N#Cc1ccc2c(c1)c1ccccc1n2-c1cc(-c2ccccc2-c2ccccc2-c2ccccc2C#N)cc(-n2c3ccccc3c3ccccc32)c1 ; N#Cc1cccc(-c2ccccc2-c2ccccc2-c2cc(-n3c4ccccc4c4ccccc43)cc(-n3c4ccccc4c4cc(C#N)ccc43)c2)c1 ; N#Cc1ccc(-c2ccccc2-c2ccccc2-c2cc(-n3c4ccccc4c4ccccc43)cc(-n3c4ccccc4c4cc(C#N)ccc43)c2)cc1]

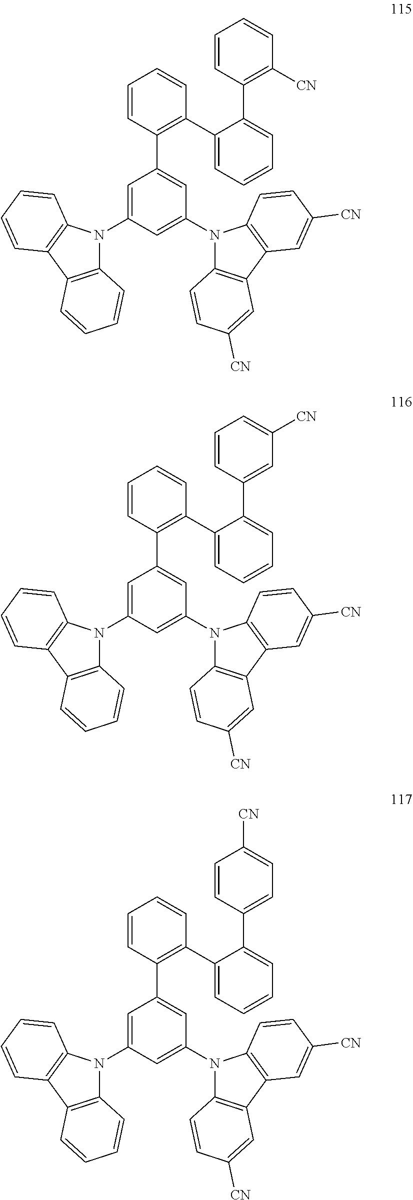 [SMILES: N#Cc1ccc2c(c1)c1cc(C#N)ccc1n2-c1cc(-c2ccccc2-c2ccccc2-c2ccccc2C#N)cc(-n2c3ccccc3c3ccccc32)c1 ; N#Cc1cccc(-c2ccccc2-c2ccccc2-c2cc(-n3c4ccccc4c4ccccc43)cc(-n3c4ccc(C#N)cc4c4cc(C#N)ccc43)c2)c1 ; N#Cc1ccc(-c2ccccc2-c2ccccc2-c2cc(-n3c4ccccc4c4ccccc43)cc(-n3c4ccc(C#N)cc4c4cc(C#N)ccc43)c2)cc1]

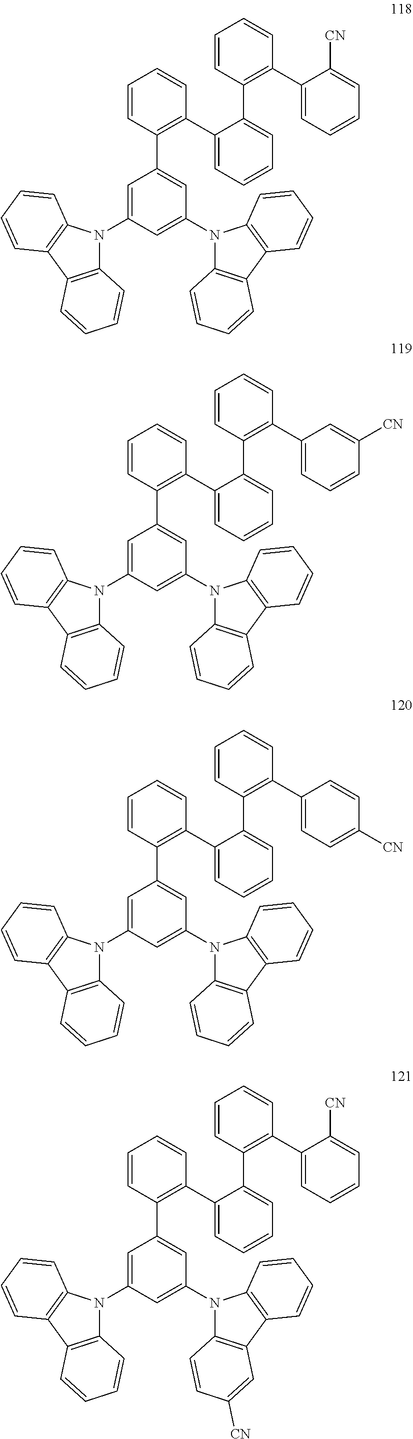

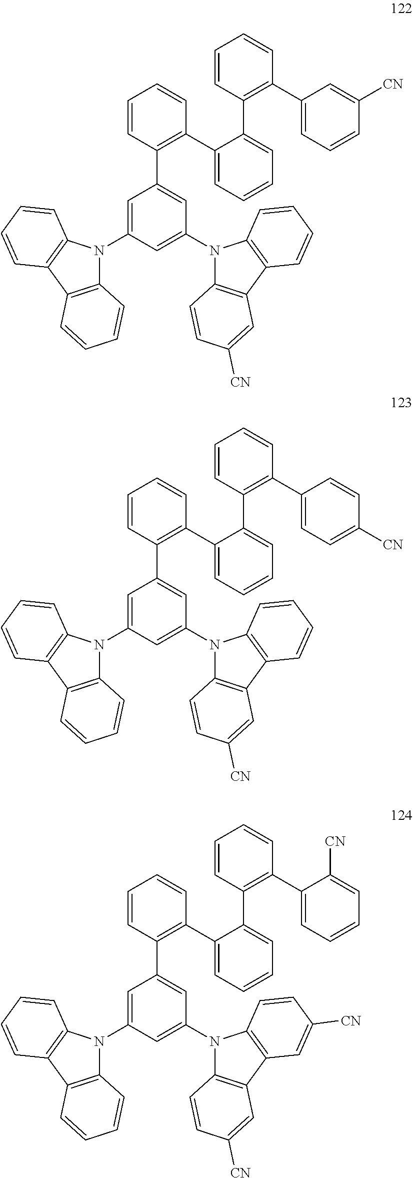 [SMILES: N#Cc1cccc(-c2ccccc2-c2ccccc2-c2ccccc2-c2cc(-n3c4ccccc4c4ccccc43)cc(-n3c4ccccc4c4cc(C#N)ccc43)c2)c1 ; N#Cc1ccc(-c2ccccc2-c2ccccc2-c2ccccc2-c2cc(-n3c4ccccc4c4ccccc43)cc(-n3c4ccccc4c4cc(C#N)ccc43)c2)cc1 ; N#Cc1ccc2c(c1)c1cc(C#N)ccc1n2-c1cc(-c2ccccc2-c2ccccc2-c2ccccc2-c2ccccc2C#N)cc(-n2c3ccccc3c3ccccc32)c1]

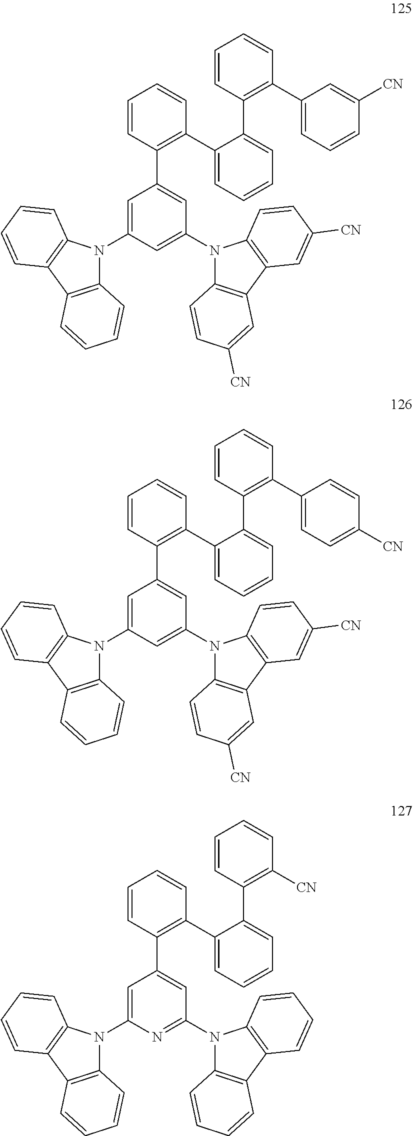

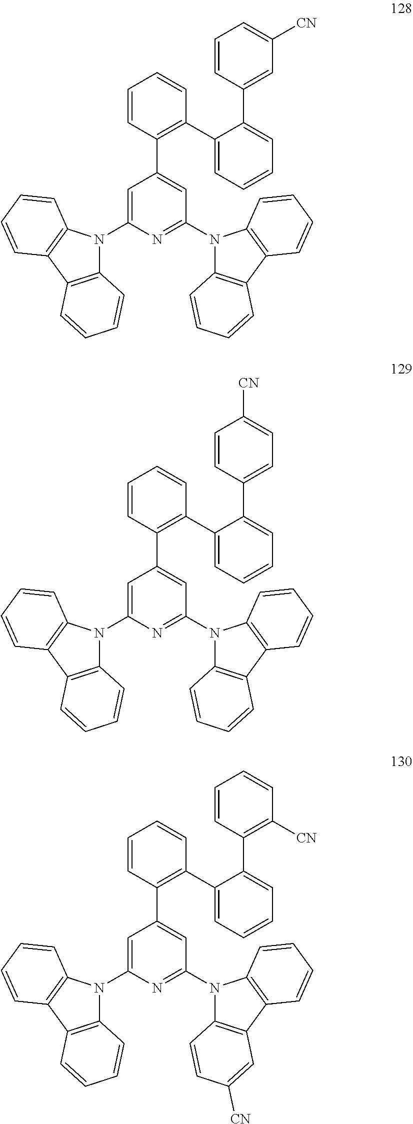

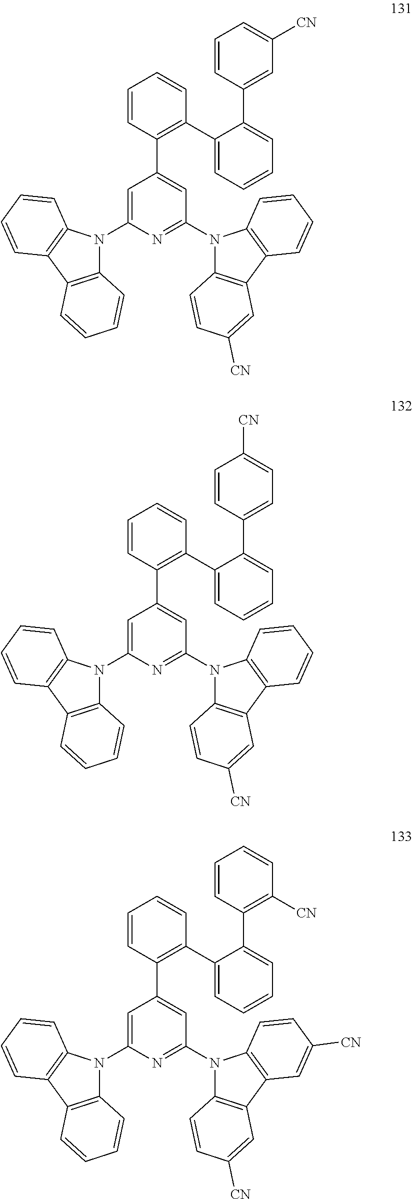

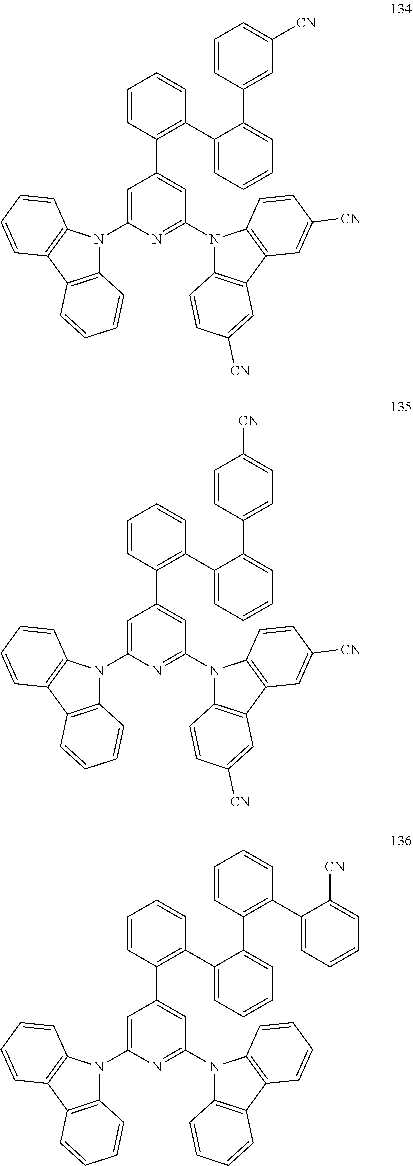 [SMILES: N#Cc1cccc(-c2ccccc2-c2ccccc2-c2cc(-n3c4ccccc4c4ccccc43)nc(-n3c4ccc(C#N)cc4c4cc(C#N)ccc43)c2)c1 ; N#Cc1ccc(-c2ccccc2-c2ccccc2-c2cc(-n3c4ccccc4c4ccccc43)nc(-n3c4ccc(C#N)cc4c4cc(C#N)ccc43)c2)cc1 ; N#Cc1ccccc1-c1ccccc1-c1ccccc1-c1ccccc1-c1cc(-n2c3ccccc3c3ccccc32)nc(-n2c3ccccc3c3ccccc32)c1]

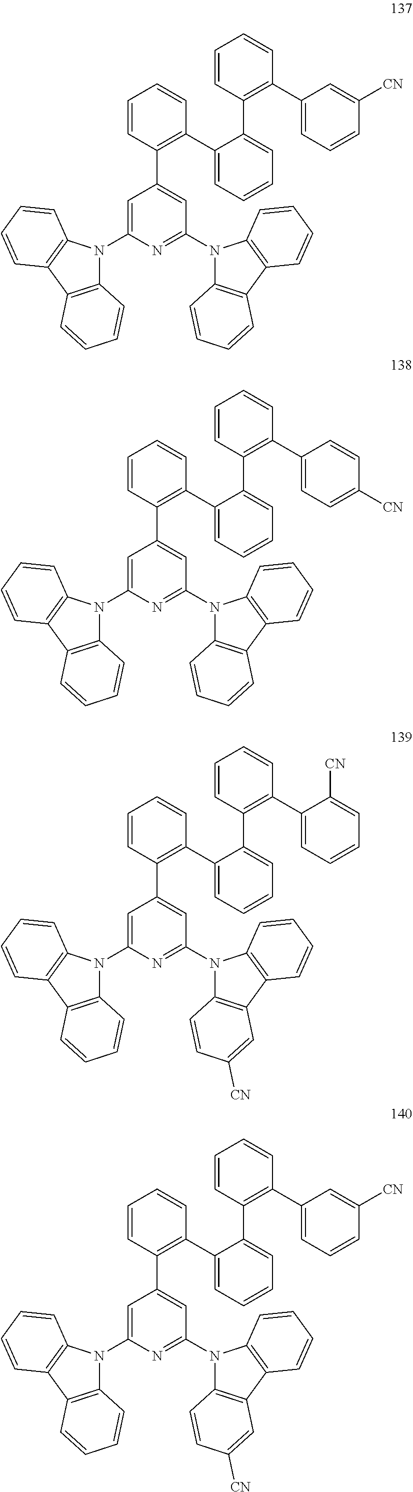

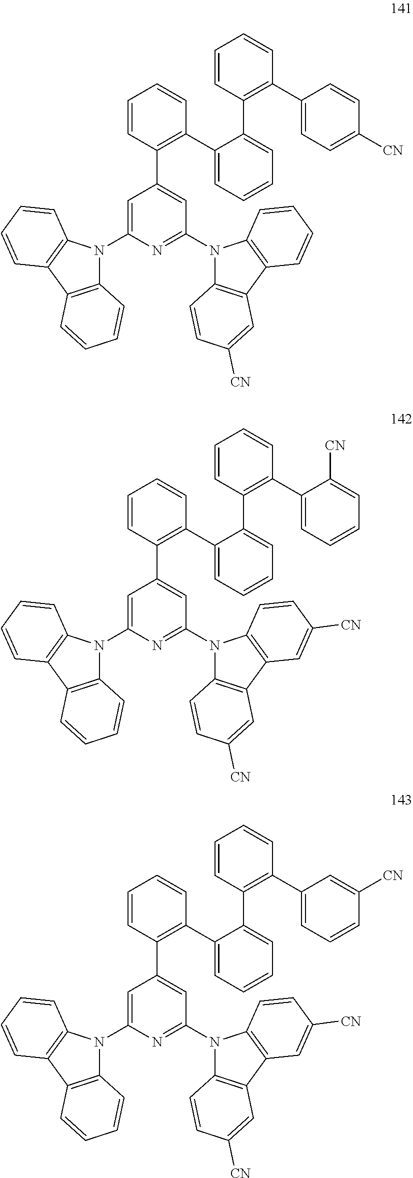 [SMILES: N#Cc1ccc(-c2ccccc2-c2ccccc2-c2ccccc2-c2cc(-n3c4ccccc4c4ccccc43)nc(-n3c4ccccc4c4cc(C#N)ccc43)c2)cc1 ; N#Cc1ccc2c(c1)c1cc(C#N)ccc1n2-c1cc(-c2ccccc2-c2ccccc2-c2ccccc2-c2ccccc2C#N)cc(-n2c3ccccc3c3ccccc32)n1 ; N#Cc1cccc(-c2ccccc2-c2ccccc2-c2ccccc2-c2cc(-n3c4ccccc4c4ccccc43)nc(-n3c4ccc(C#N)cc4c4cc(C#N)ccc43)c2)c1]

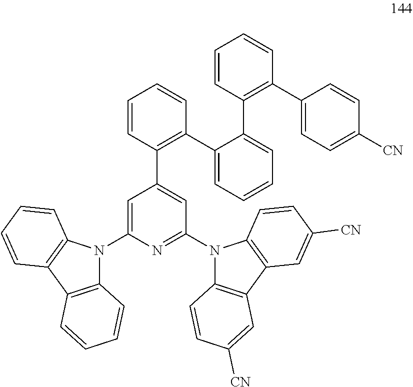 [SMILES: N#Cc1ccc(-c2ccccc2-c2ccccc2-c2ccccc2-c2cc(-n3c4ccccc4c4ccccc43)nc(-n3c4ccc(C#N)cc4c4cc(C#N)ccc43)c2)cc1]

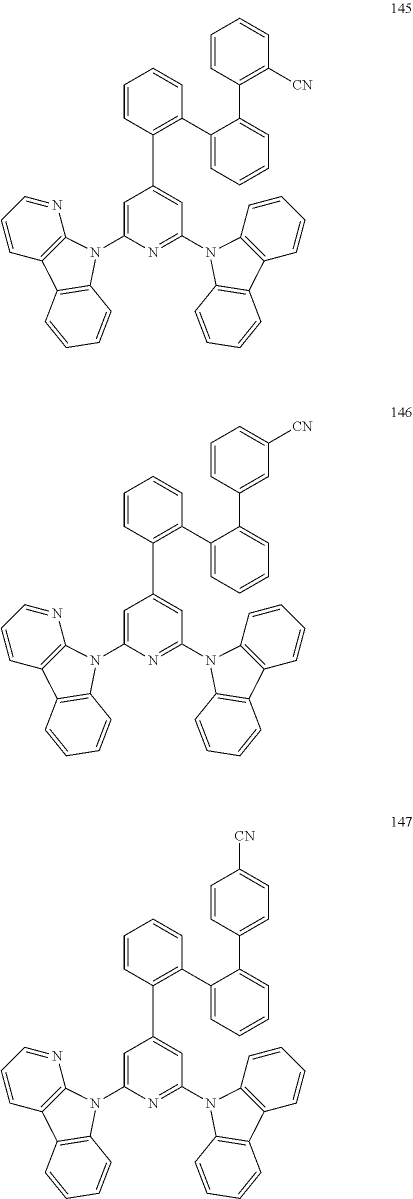 [SMILES: N#Cc1ccccc1-c1ccccc1-c1ccccc1-c1cc(-n2c3ccccc3c3ccccc32)nc(-n2c3ccccc3c3cccnc32)c1 ; N#Cc1cccc(-c2ccccc2-c2ccccc2-c2cc(-n3c4ccccc4c4ccccc43)nc(-n3c4ccccc4c4cccnc43)c2)c1 ; N#Cc1ccc(-c2ccccc2-c2ccccc2-c2cc(-n3c4ccccc4c4ccccc43)nc(-n3c4ccccc4c4cccnc43)c2)cc1]

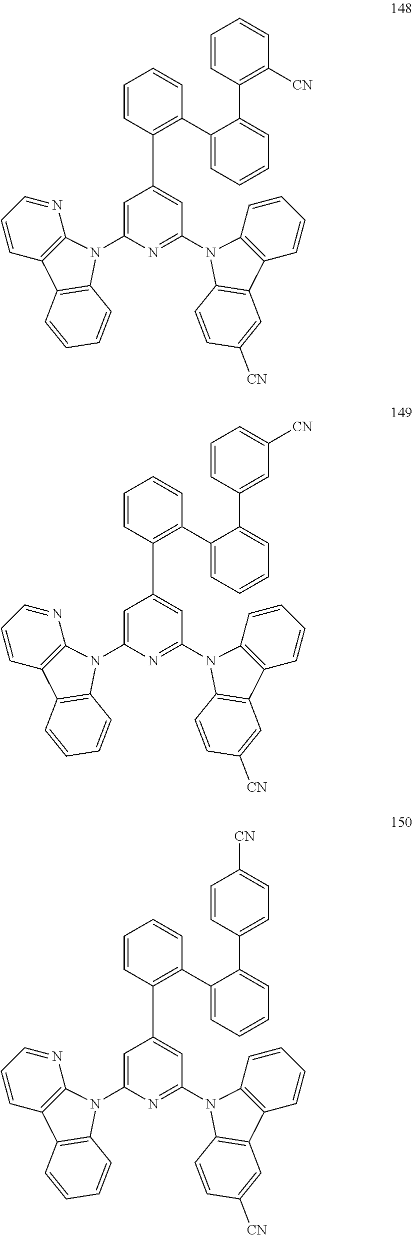 [SMILES: N#Cc1ccc2c(c1)c1ccccc1n2-c1cc(-c2ccccc2-c2ccccc2-c2ccccc2C#N)cc(-n2c3ccccc3c3cccnc32)n1 ; N#Cc1cccc(-c2ccccc2-c2ccccc2-c2cc(-n3c4ccccc4c4cc(C#N)ccc43)nc(-n3c4ccccc4c4cccnc43)c2)c1 ; N#Cc1ccc(-c2ccccc2-c2ccccc2-c2cc(-n3c4ccccc4c4cc(C#N)ccc43)nc(-n3c4ccccc4c4cccnc43)c2)cc1]

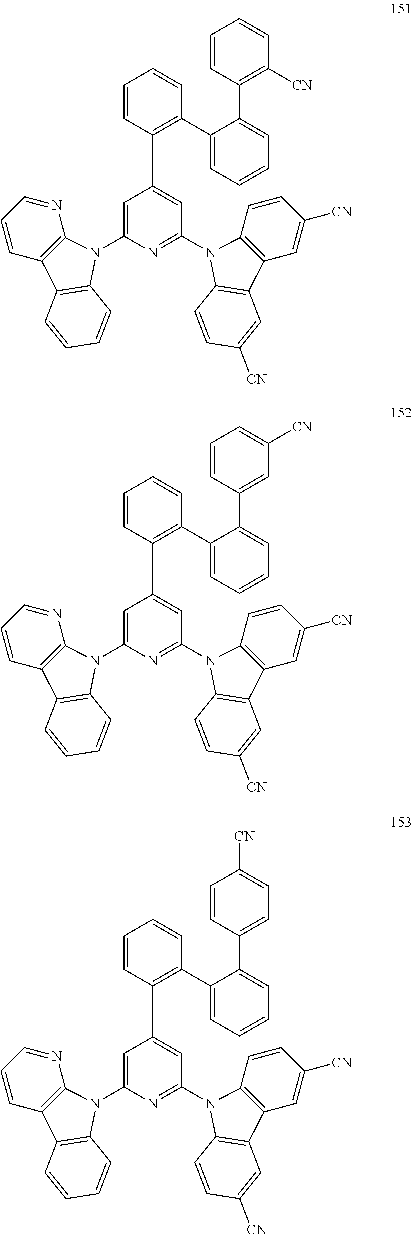 [SMILES: N#Cc1ccc2c(c1)c1cc(C#N)ccc1n2-c1cc(-c2ccccc2-c2ccccc2-c2ccccc2C#N)cc(-n2c3ccccc3c3cccnc32)n1 ; N#Cc1cccc(-c2ccccc2-c2ccccc2-c2cc(-n3c4ccc(C#N)cc4c4cc(C#N)ccc43)nc(-n3c4ccccc4c4cccnc43)c2)c1 ; N#Cc1ccc(-c2ccccc2-c2ccccc2-c2cc(-n3c4ccc(C#N)cc4c4cc(C#N)ccc43)nc(-n3c4ccccc4c4cccnc43)c2)cc1]

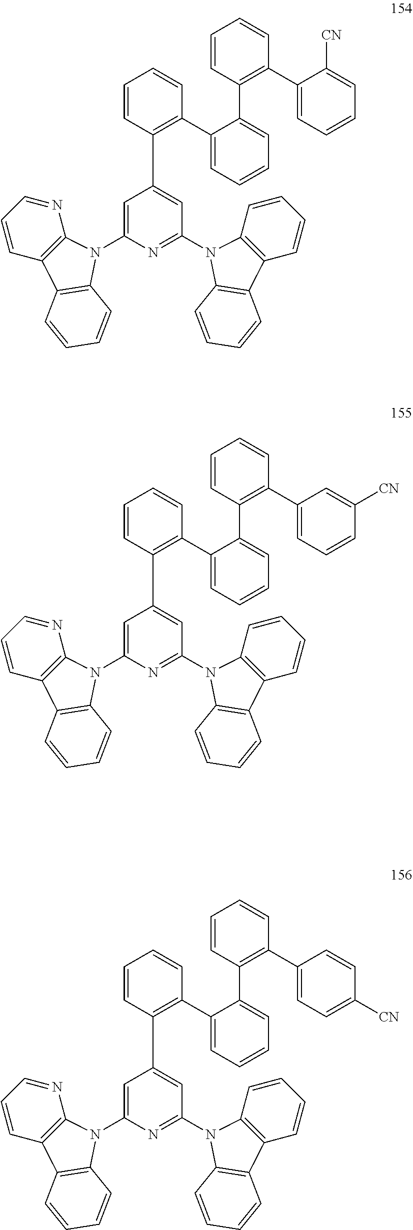 [SMILES: N#Cc1ccccc1-c1ccccc1-c1ccccc1-c1ccccc1-c1cc(-n2c3ccccc3c3ccccc32)nc(-n2c3ccccc3c3cccnc32)c1 ; N#Cc1cccc(-c2ccccc2-c2ccccc2-c2ccccc2-c2cc(-n3c4ccccc4c4ccccc43)nc(-n3c4ccccc4c4cccnc43)c2)c1 ; N#Cc1ccc(-c2ccccc2-c2ccccc2-c2ccccc2-c2cc(-n3c4ccccc4c4ccccc43)nc(-n3c4ccccc4c4cccnc43)c2)cc1]

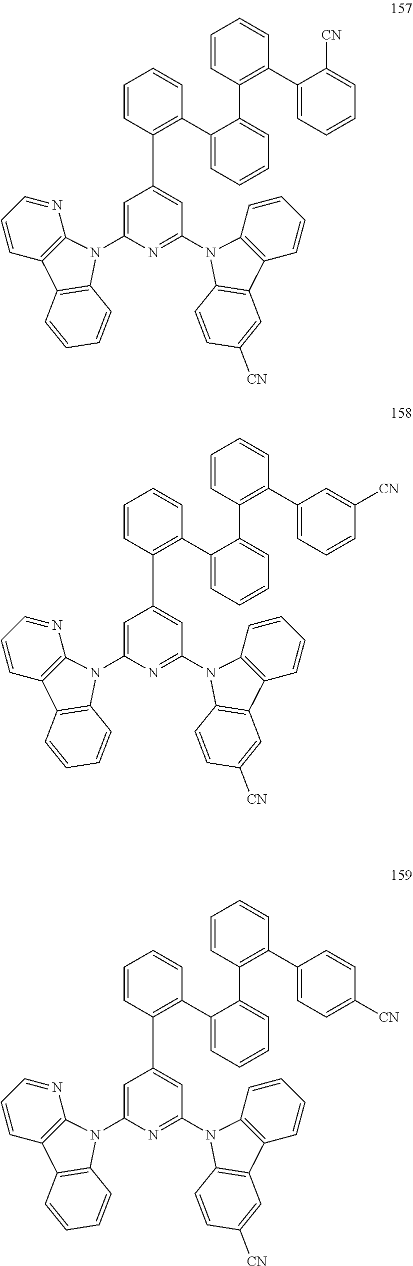 [SMILES: N#Cc1ccc2c(c1)c1ccccc1n2-c1cc(-c2ccccc2-c2ccccc2-c2ccccc2-c2ccccc2C#N)cc(-n2c3ccccc3c3cccnc32)n1 ; N#Cc1cccc(-c2ccccc2-c2ccccc2-c2ccccc2-c2cc(-n3c4ccccc4c4cc(C#N)ccc43)nc(-n3c4ccccc4c4cccnc43)c2)c1 ; N#Cc1ccc(-c2ccccc2-c2ccccc2-c2ccccc2-c2cc(-n3c4ccccc4c4cc(C#N)ccc43)nc(-n3c4ccccc4c4cccnc43)c2)cc1]

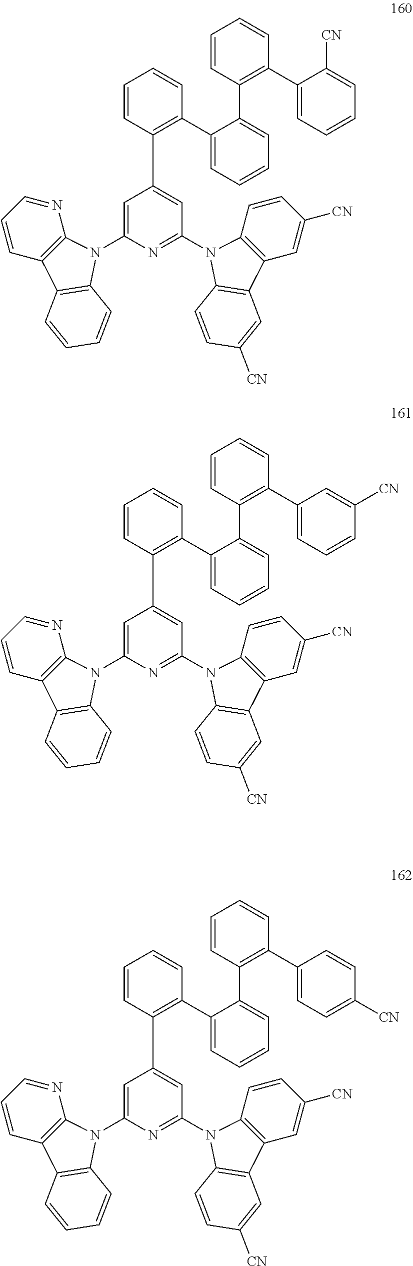 [SMILES: N#Cc1ccc2c(c1)c1cc(C#N)ccc1n2-c1cc(-c2ccccc2-c2ccccc2-c2ccccc2-c2ccccc2C#N)cc(-n2c3ccccc3c3cccnc32)n1 ; N#Cc1cccc(-c2ccccc2-c2ccccc2-c2ccccc2-c2cc(-n3c4ccc(C#N)cc4c4cc(C#N)ccc43)nc(-n3c4ccccc4c4cccnc43)c2)c1 ; N#Cc1ccc(-c2ccccc2-c2ccccc2-c2ccccc2-c2cc(-n3c4ccc(C#N)cc4c4cc(C#N)ccc43)nc(-n3c4ccccc4c4cccnc43)c2)cc1]

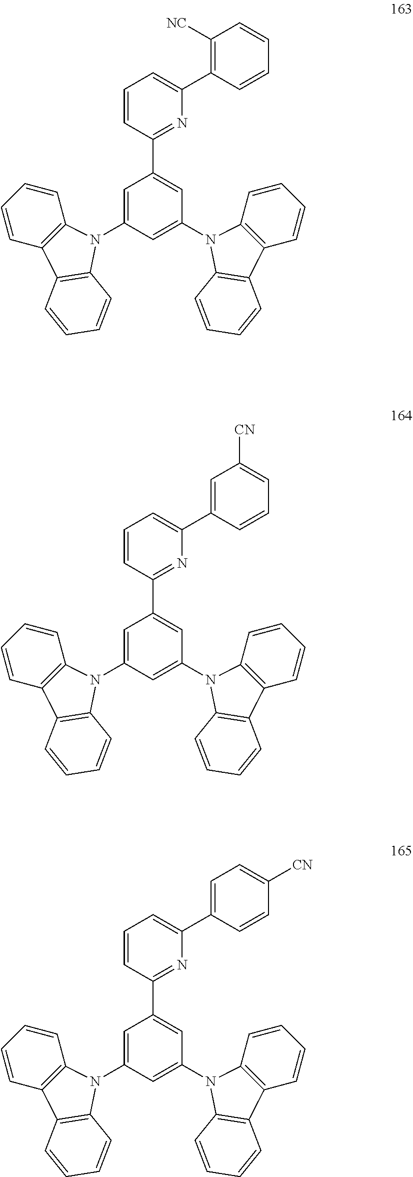 [SMILES: N#Cc1ccccc1-c1cccc(-c2cc(-n3c4ccccc4c4ccccc43)cc(-n3c4ccccc4c4ccccc43)c2)n1 ; N#Cc1cccc(-c2cccc(-c3cc(-n4c5ccccc5c5ccccc54)cc(-n4c5ccccc5c5ccccc54)c3)n2)c1 ; N#Cc1ccc(-c2cccc(-c3cc(-n4c5ccccc5c5ccccc54)cc(-n4c5ccccc5c5ccccc54)c3)n2)cc1]

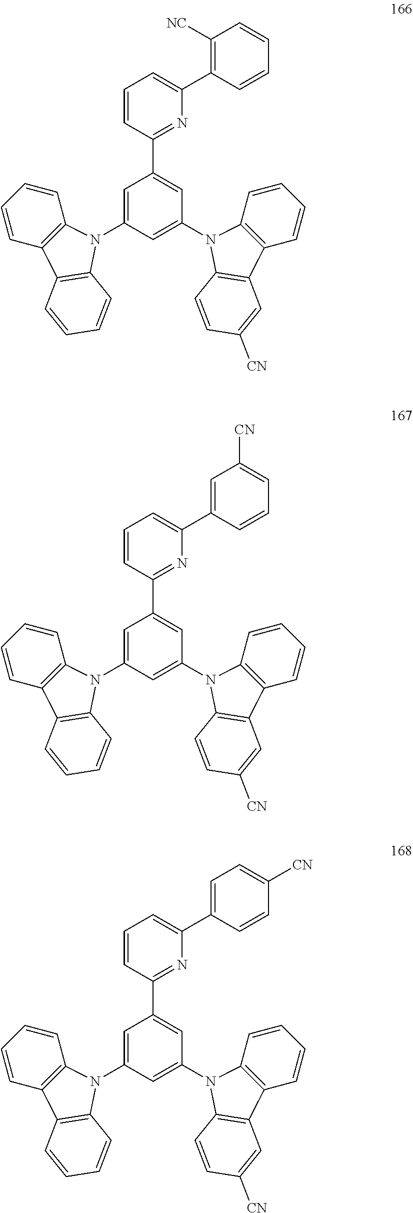 [SMILES: N#Cc1ccc2c(c1)c1ccccc1n2-c1cc(-c2cccc(-c3ccccc3C#N)n2)cc(-n2c3ccccc3c3ccccc32)c1 ; N#Cc1cccc(-c2cccc(-c3cc(-n4c5ccccc5c5ccccc54)cc(-n4c5ccccc5c5cc(C#N)ccc54)c3)n2)c1 ; N#Cc1ccc(-c2cccc(-c3cc(-n4c5ccccc5c5ccccc54)cc(-n4c5ccccc5c5cc(C#N)ccc54)c3)n2)cc1]

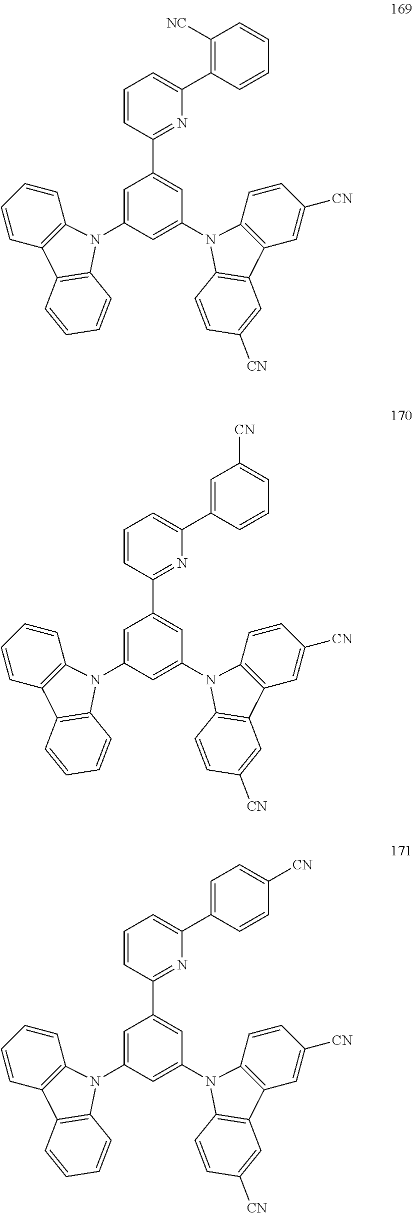 [SMILES: N#Cc1ccc2c(c1)c1cc(C#N)ccc1n2-c1cc(-c2cccc(-c3ccccc3C#N)n2)cc(-n2c3ccccc3c3ccccc32)c1 ; N#Cc1cccc(-c2cccc(-c3cc(-n4c5ccccc5c5ccccc54)cc(-n4c5ccc(C#N)cc5c5cc(C#N)ccc54)c3)n2)c1 ; N#Cc1ccc(-c2cccc(-c3cc(-n4c5ccccc5c5ccccc54)cc(-n4c5ccc(C#N)cc5c5cc(C#N)ccc54)c3)n2)cc1]

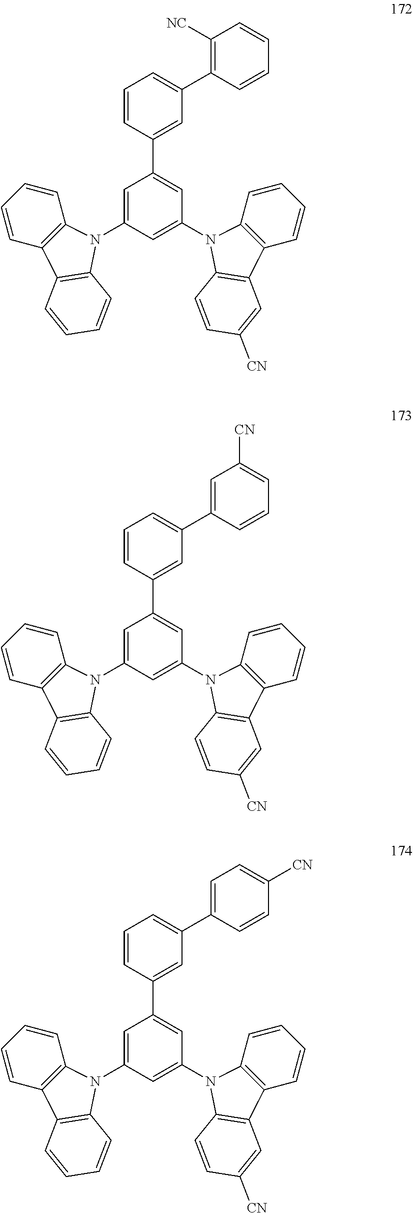 [SMILES: N#Cc1ccc2c(c1)c1ccccc1n2-c1cc(-c2cccc(-c3ccccc3C#N)c2)cc(-n2c3ccccc3c3ccccc32)c1 ; N#Cc1cccc(-c2cccc(-c3cc(-n4c5ccccc5c5ccccc54)cc(-n4c5ccccc5c5cc(C#N)ccc54)c3)c2)c1 ; N#Cc1ccc(-c2cccc(-c3cc(-n4c5ccccc5c5ccccc54)cc(-n4c5ccccc5c5cc(C#N)ccc54)c3)c2)cc1]

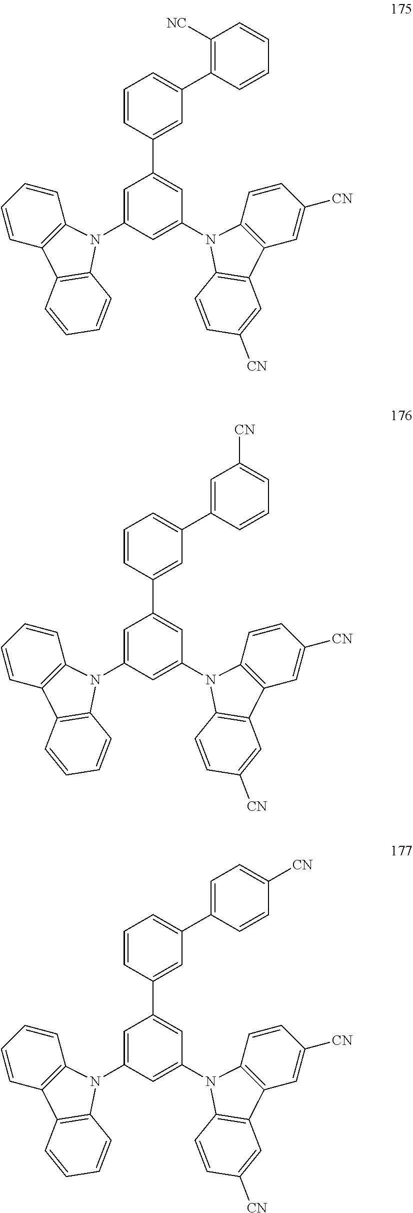 [SMILES: N#Cc1ccc2c(c1)c1cc(C#N)ccc1n2-c1cc(-c2cccc(-c3ccccc3C#N)c2)cc(-n2c3ccccc3c3ccccc32)c1 ; N#Cc1cccc(-c2cccc(-c3cc(-n4c5ccccc5c5ccccc54)cc(-n4c5ccc(C#N)cc5c5cc(C#N)ccc54)c3)c2)c1 ; N#Cc1ccc(-c2cccc(-c3cc(-n4c5ccccc5c5ccccc54)cc(-n4c5ccc(C#N)cc5c5cc(C#N)ccc54)c3)c2)cc1]

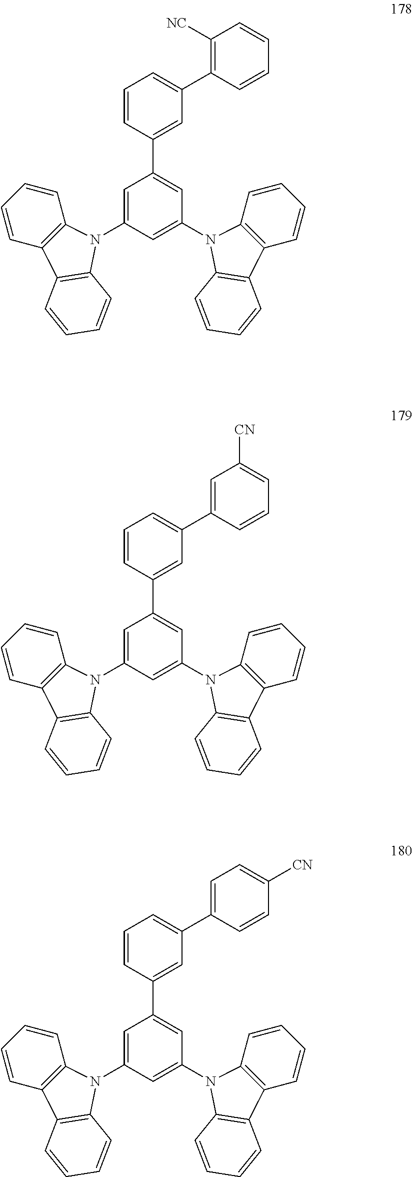 [SMILES: N#Cc1ccccc1-c1cccc(-c2cc(-n3c4ccccc4c4ccccc43)cc(-n3c4ccccc4c4ccccc43)c2)c1 ; N#Cc1cccc(-c2cccc(-c3cc(-n4c5ccccc5c5ccccc54)cc(-n4c5ccccc5c5ccccc54)c3)c2)c1 ; N#Cc1ccc(-c2cccc(-c3cc(-n4c5ccccc5c5ccccc54)cc(-n4c5ccccc5c5ccccc54)c3)c2)cc1]

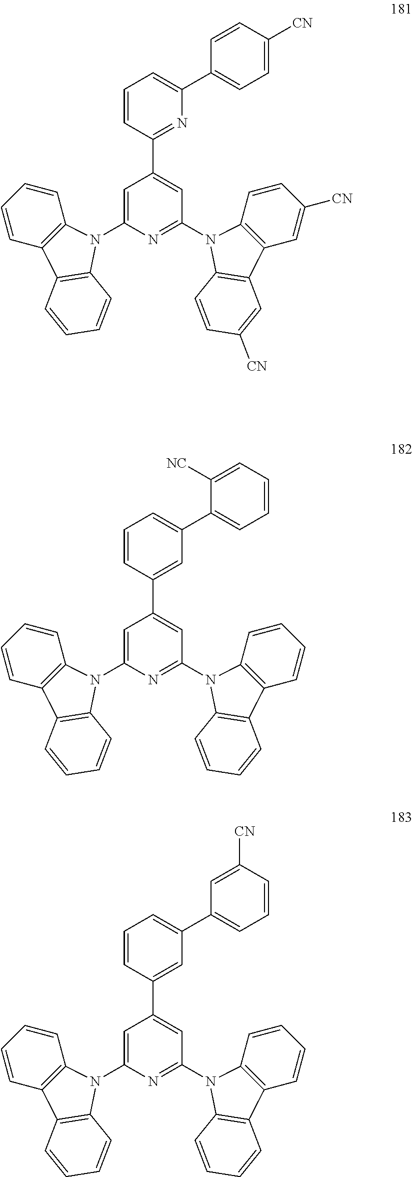 [SMILES: N#Cc1ccc(-c2cccc(-c3cc(-n4c5ccccc5c5ccccc54)nc(-n4c5ccc(C#N)cc5c5cc(C#N)ccc54)c3)n2)cc1 ; N#Cc1ccccc1-c1cccc(-c2cc(-n3c4ccccc4c4ccccc43)nc(-n3c4ccccc4c4ccccc43)c2)c1 ; N#Cc1cccc(-c2cccc(-c3cc(-n4c5ccccc5c5ccccc54)nc(-n4c5ccccc5c5ccccc54)c3)c2)c1]

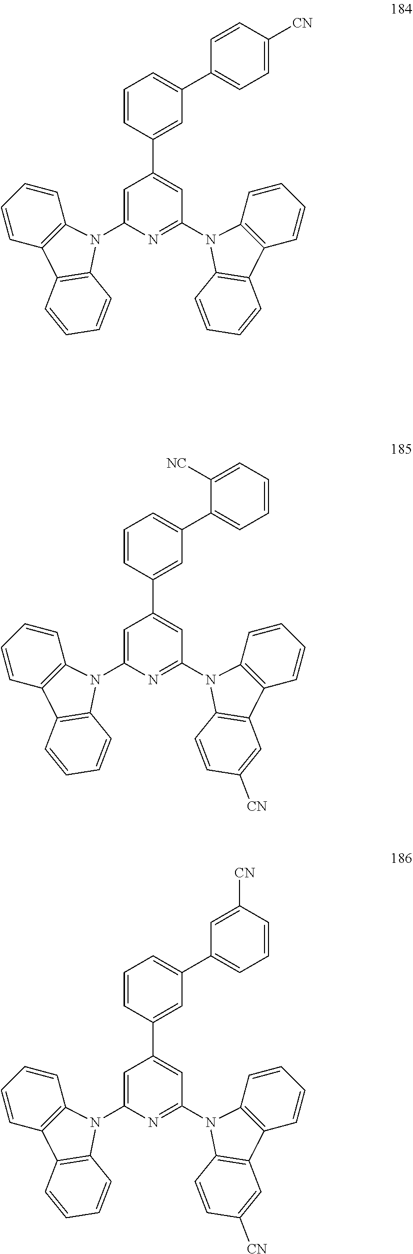 [SMILES: N#Cc1ccc(-c2cccc(-c3cc(-n4c5ccccc5c5ccccc54)nc(-n4c5ccccc5c5ccccc54)c3)c2)cc1 ; N#Cc1ccc2c(c1)c1ccccc1n2-c1cc(-c2cccc(-c3ccccc3C#N)c2)cc(-n2c3ccccc3c3ccccc32)n1 ; N#Cc1cccc(-c2cccc(-c3cc(-n4c5ccccc5c5ccccc54)nc(-n4c5ccccc5c5cc(C#N)ccc54)c3)c2)c1]

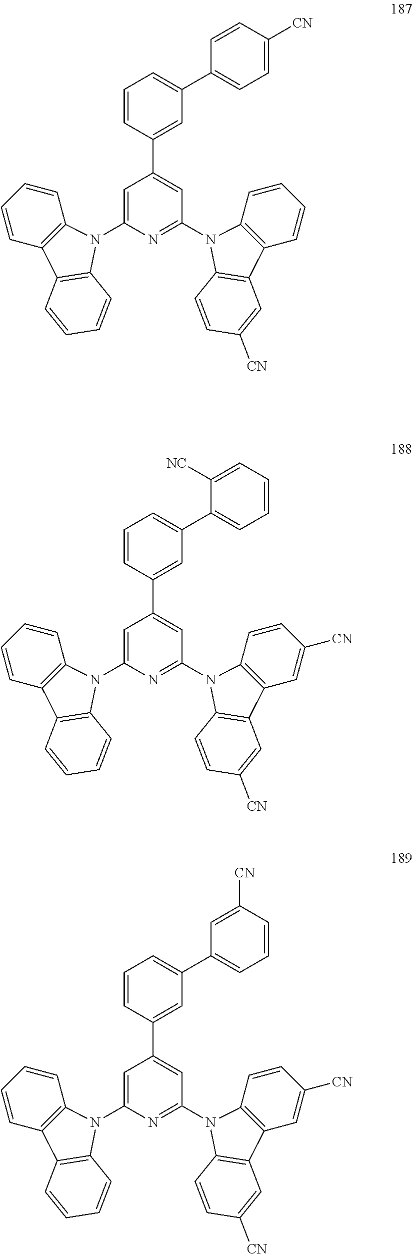 [SMILES: N#Cc1ccc(-c2cccc(-c3cc(-n4c5ccccc5c5ccccc54)nc(-n4c5ccccc5c5cc(C#N)ccc54)c3)c2)cc1 ; N#Cc1ccc2c(c1)c1cc(C#N)ccc1n2-c1cc(-c2cccc(-c3ccccc3C#N)c2)cc(-n2c3ccccc3c3ccccc32)n1 ; N#Cc1cccc(-c2cccc(-c3cc(-n4c5ccccc5c5ccccc54)nc(-n4c5ccc(C#N)cc5c5cc(C#N)ccc54)c3)c2)c1]

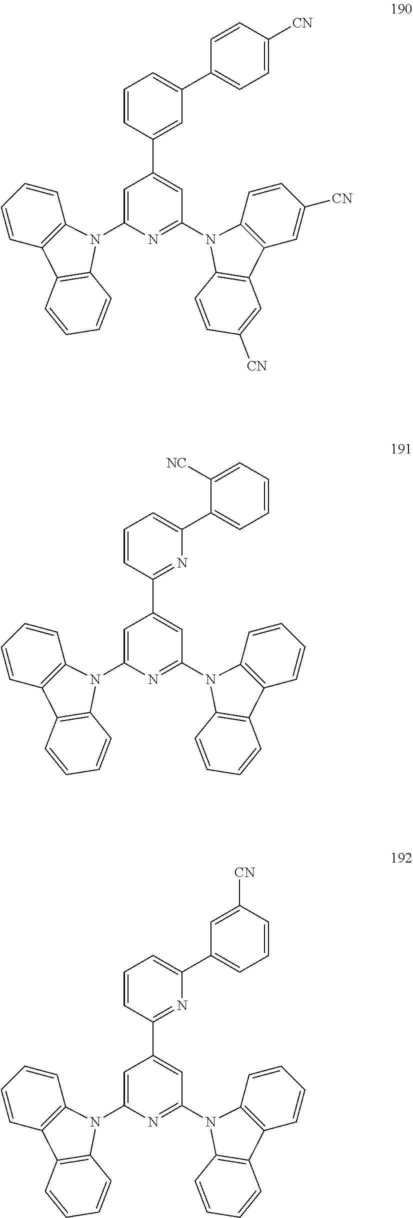 [SMILES: N#Cc1ccc(-c2cccc(-c3cc(-n4c5ccccc5c5ccccc54)nc(-n4c5ccc(C#N)cc5c5cc(C#N)ccc54)c3)c2)cc1 ; N#Cc1ccccc1-c1cccc(-c2cc(-n3c4ccccc4c4ccccc43)nc(-n3c4ccccc4c4ccccc43)c2)n1 ; N#Cc1cccc(-c2cccc(-c3cc(-n4c5ccccc5c5ccccc54)nc(-n4c5ccccc5c5ccccc54)c3)n2)c1]

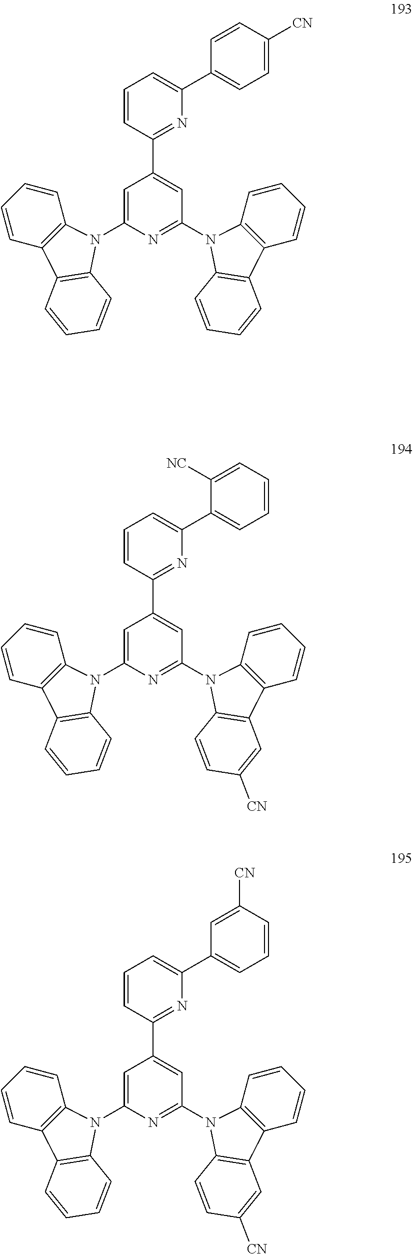 [SMILES: N#Cc1ccc(-c2cccc(-c3cc(-n4c5ccccc5c5ccccc54)nc(-n4c5ccccc5c5ccccc54)c3)n2)cc1 ; N#Cc1ccc2c(c1)c1ccccc1n2-c1cc(-c2cccc(-c3ccccc3C#N)n2)cc(-n2c3ccccc3c3ccccc32)n1 ; N#Cc1cccc(-c2cccc(-c3cc(-n4c5ccccc5c5ccccc54)nc(-n4c5ccccc5c5cc(C#N)ccc54)c3)n2)c1]

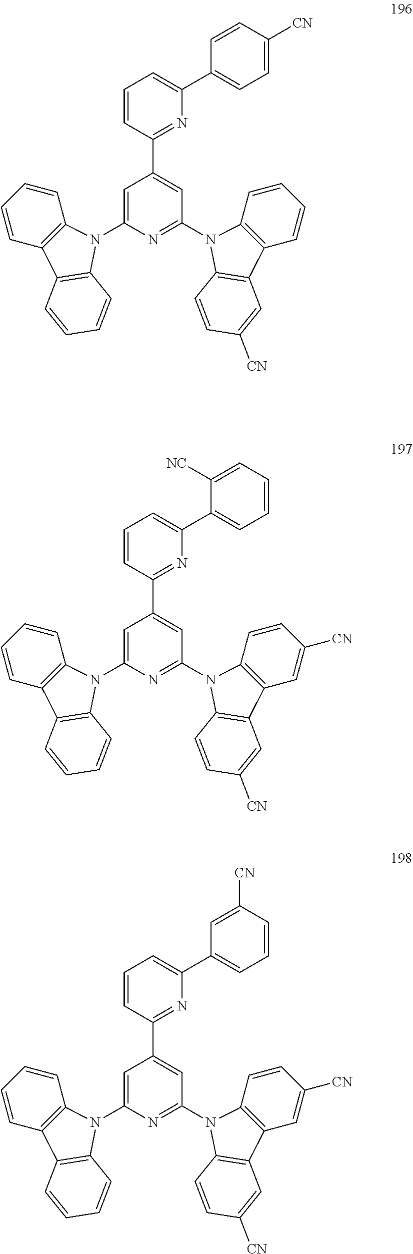 [SMILES: N#Cc1ccc(-c2cccc(-c3cc(-n4c5ccccc5c5ccccc54)nc(-n4c5ccccc5c5cc(C#N)ccc54)c3)n2)cc1 ; N#Cc1ccc2c(c1)c1cc(C#N)ccc1n2-c1cc(-c2cccc(-c3ccccc3C#N)n2)cc(-n2c3ccccc3c3ccccc32)n1 ; N#Cc1cccc(-c2cccc(-c3cc(-n4c5ccccc5c5ccccc54)nc(-n4c5ccc(C#N)cc5c5cc(C#N)ccc54)c3)n2)c1]

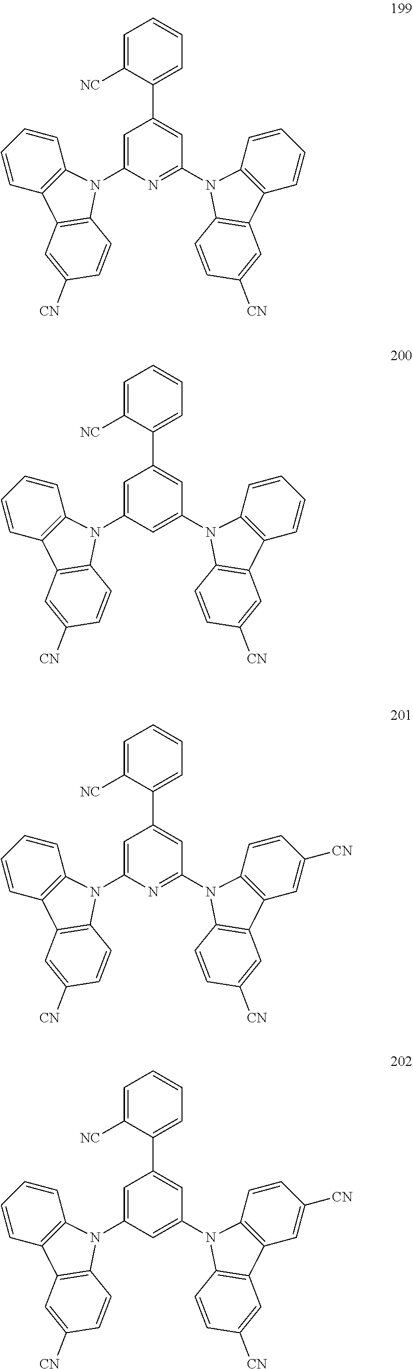

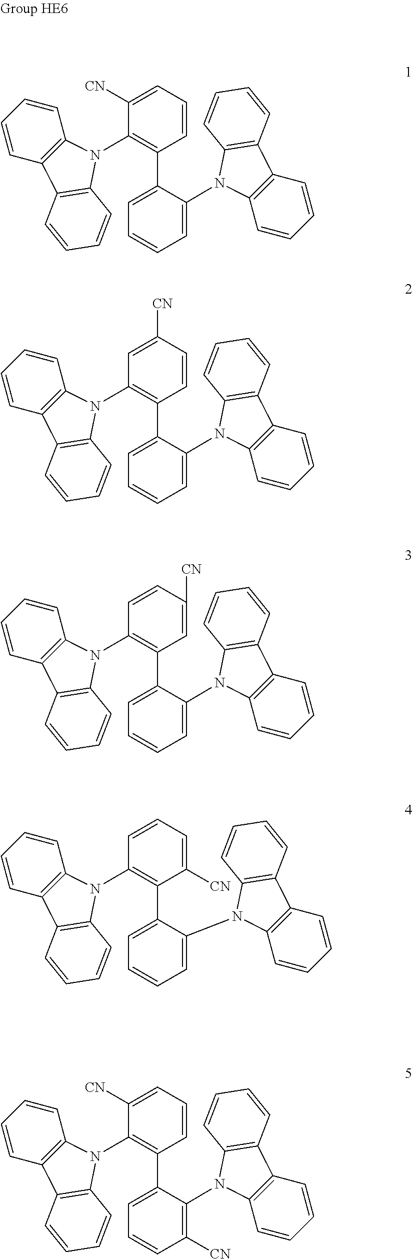

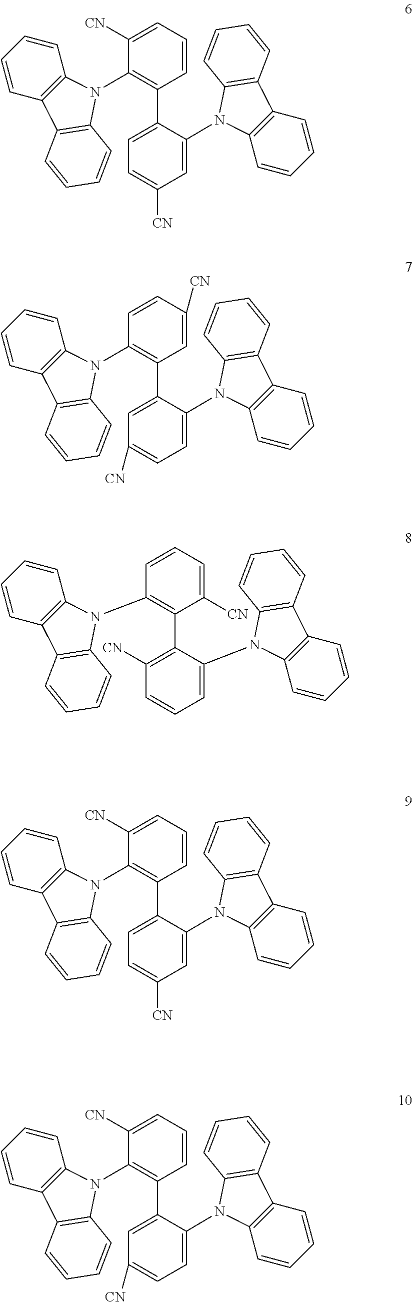

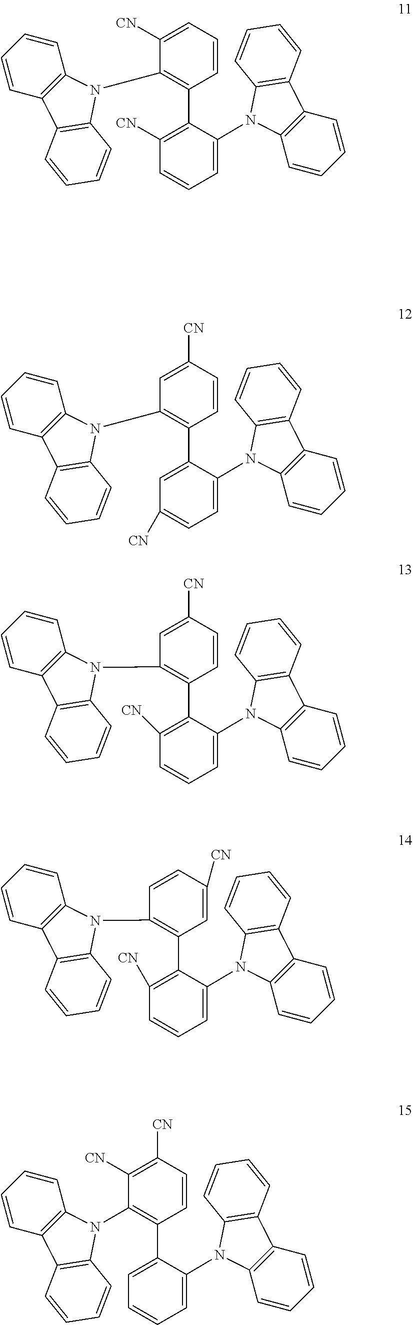

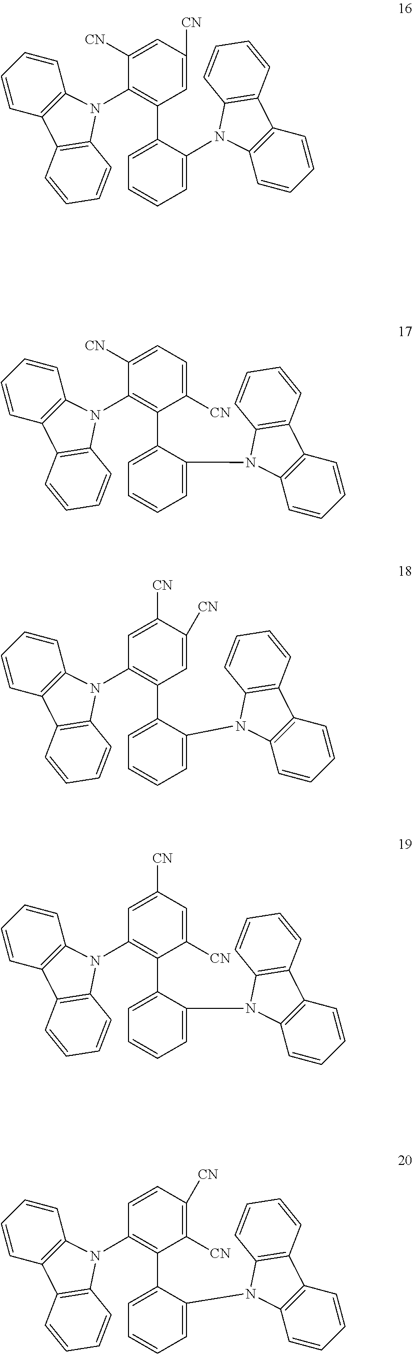

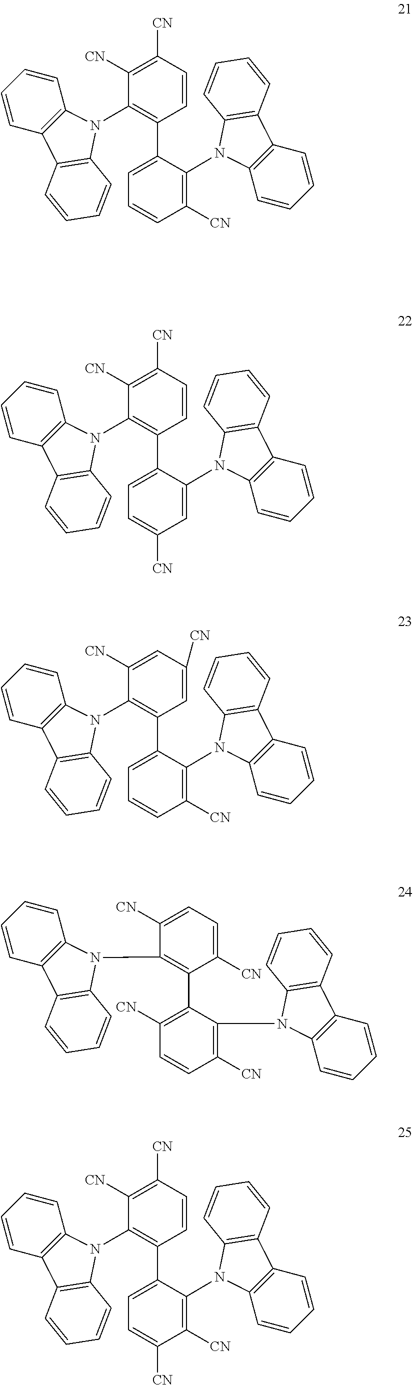

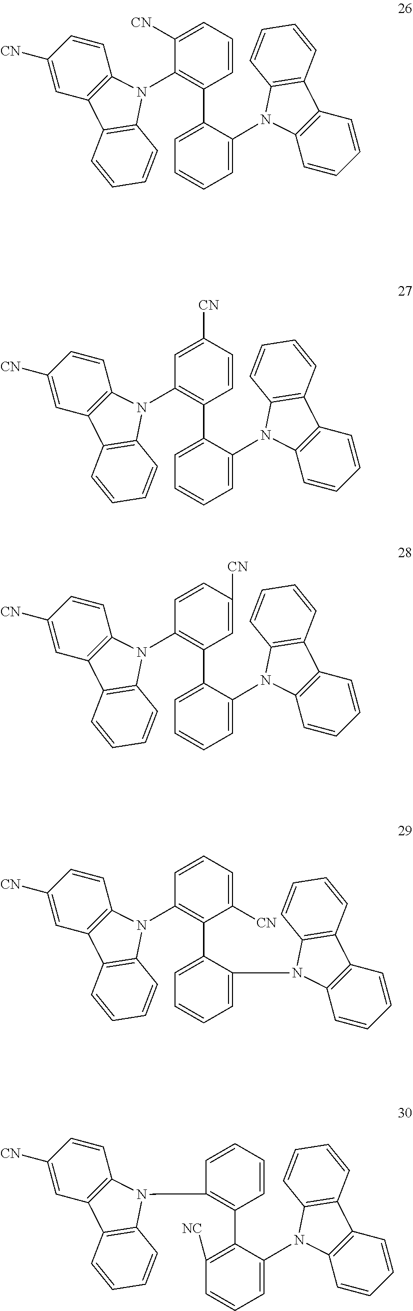

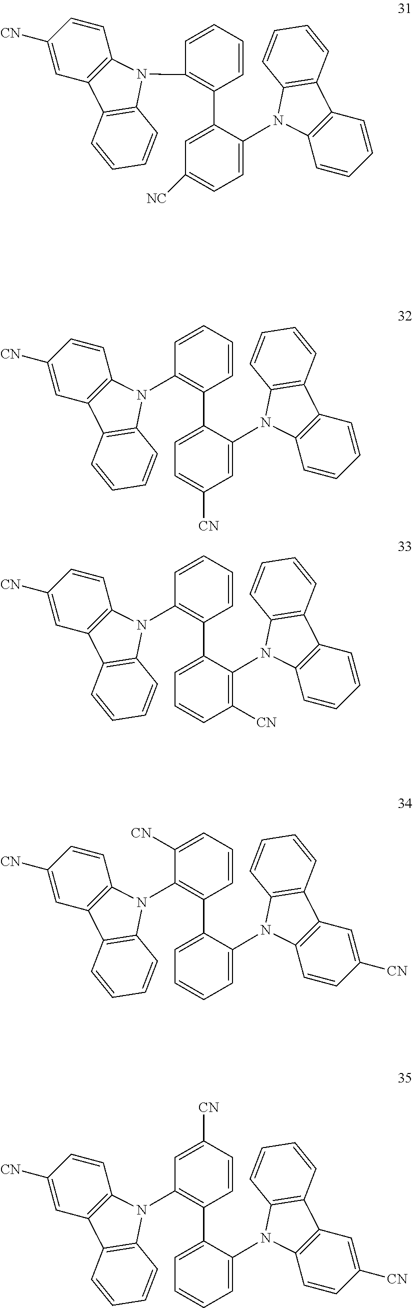

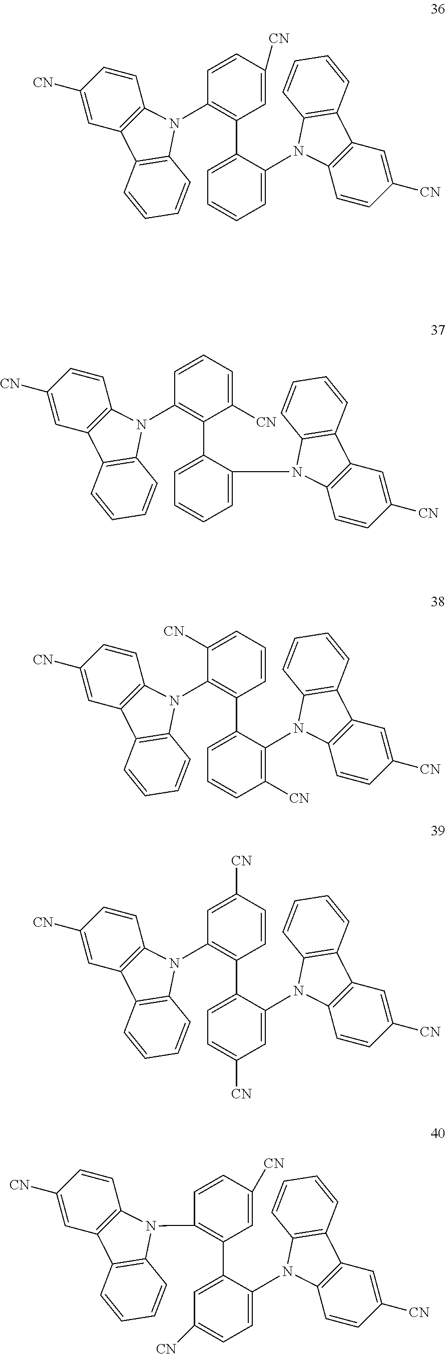

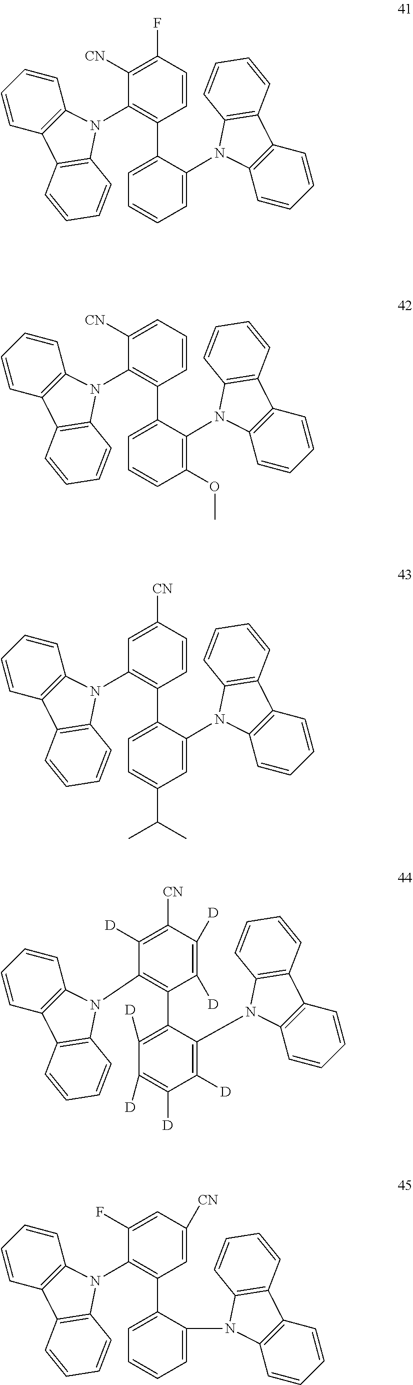

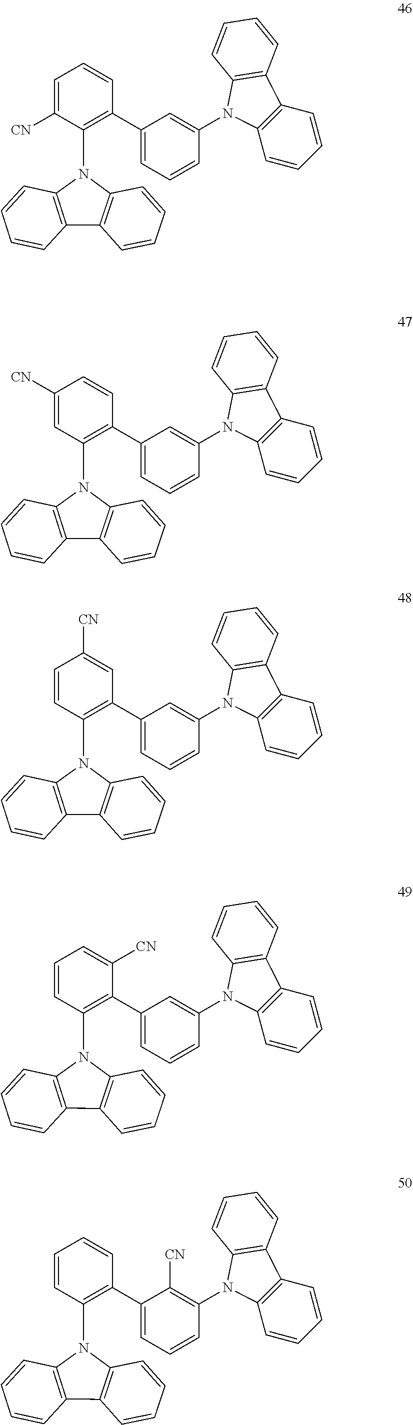

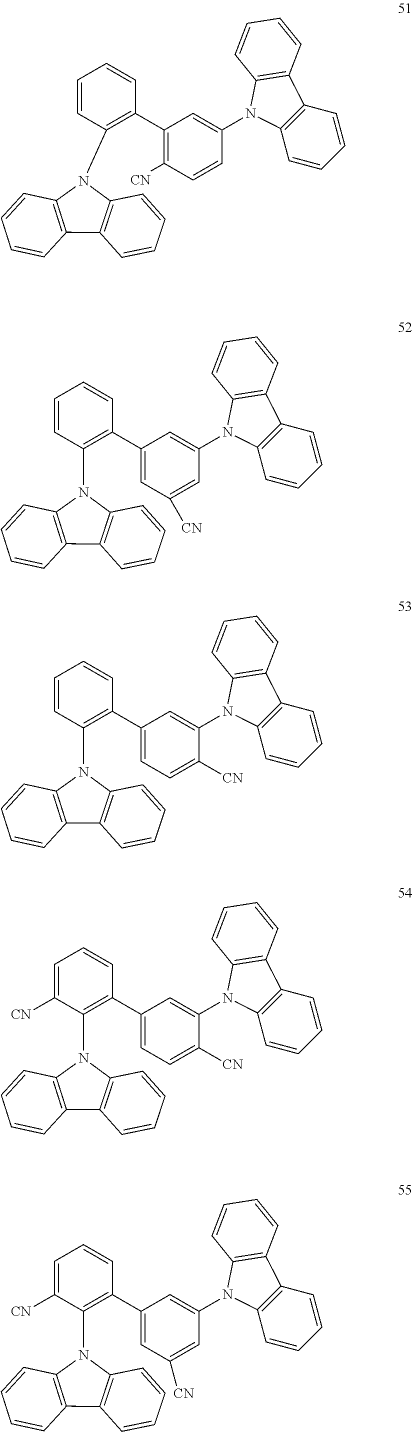

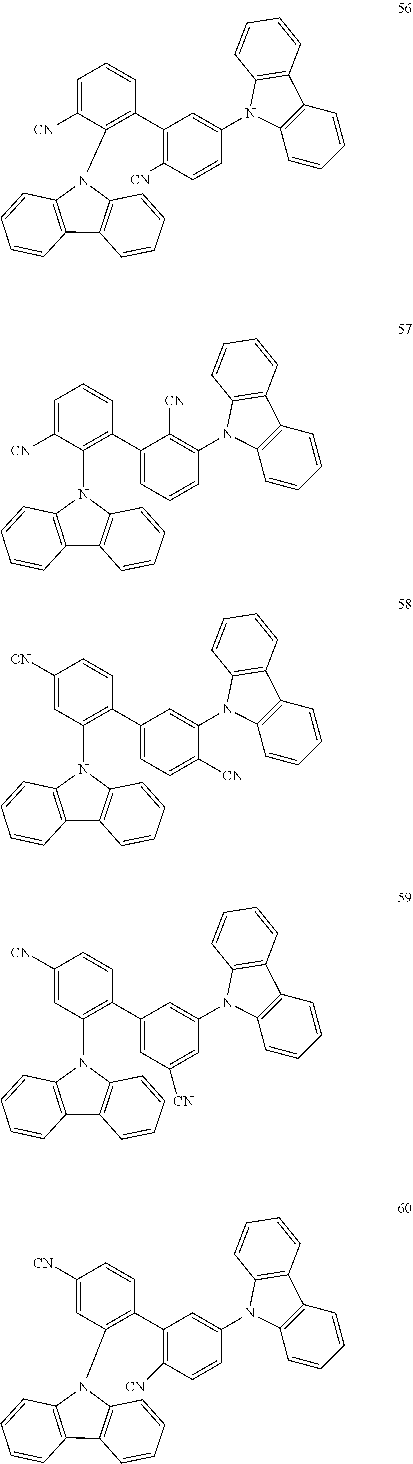

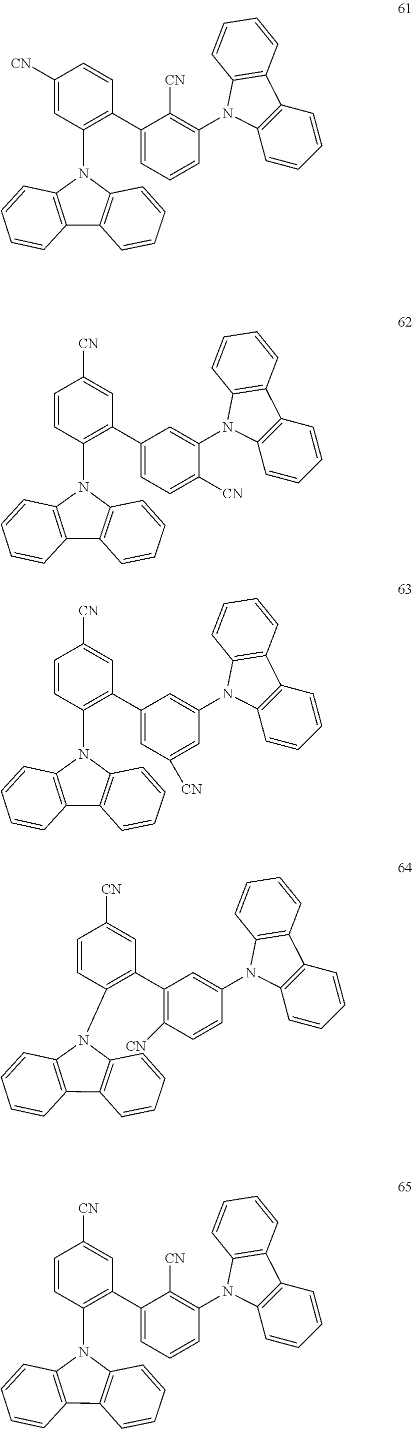

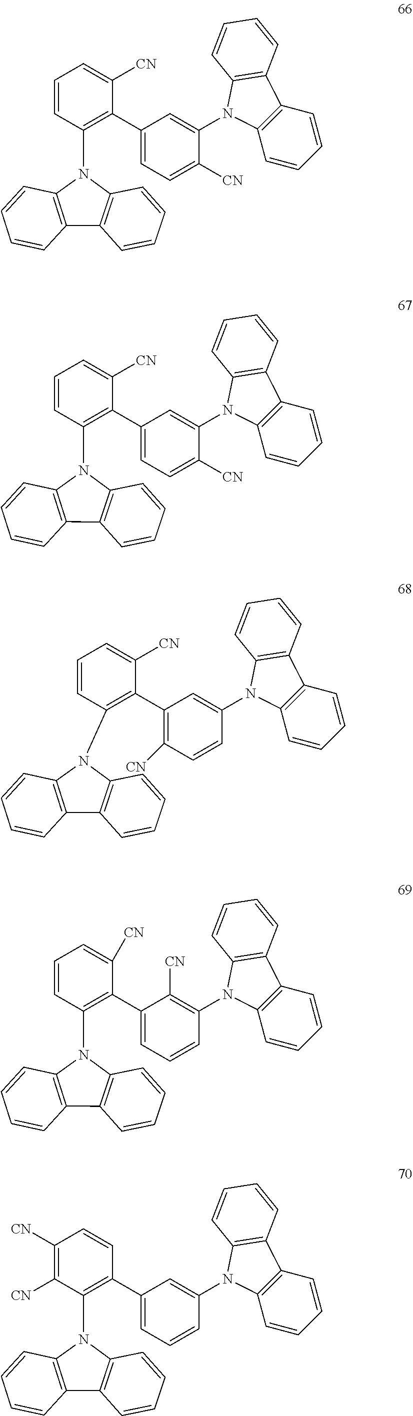

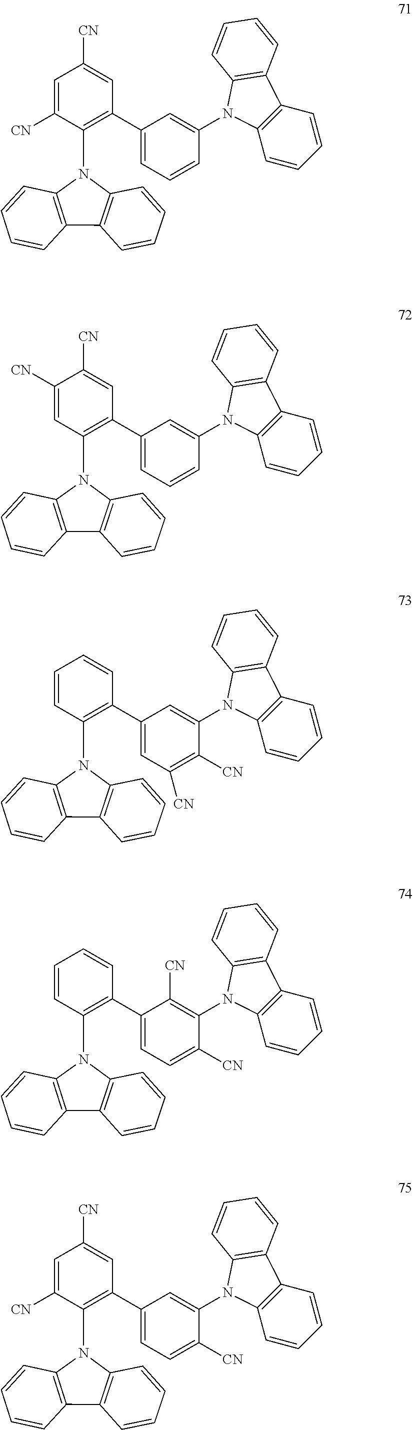

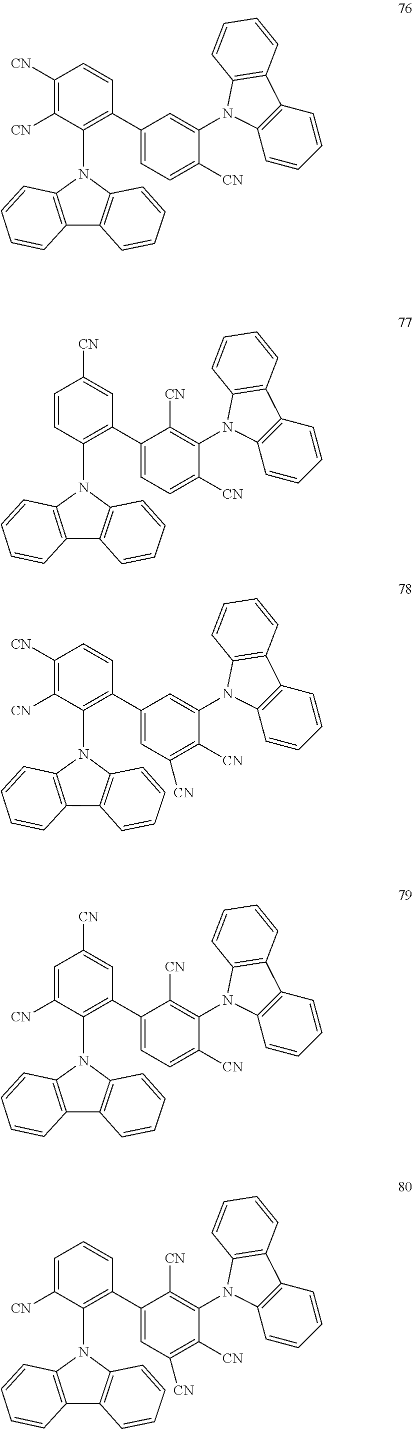

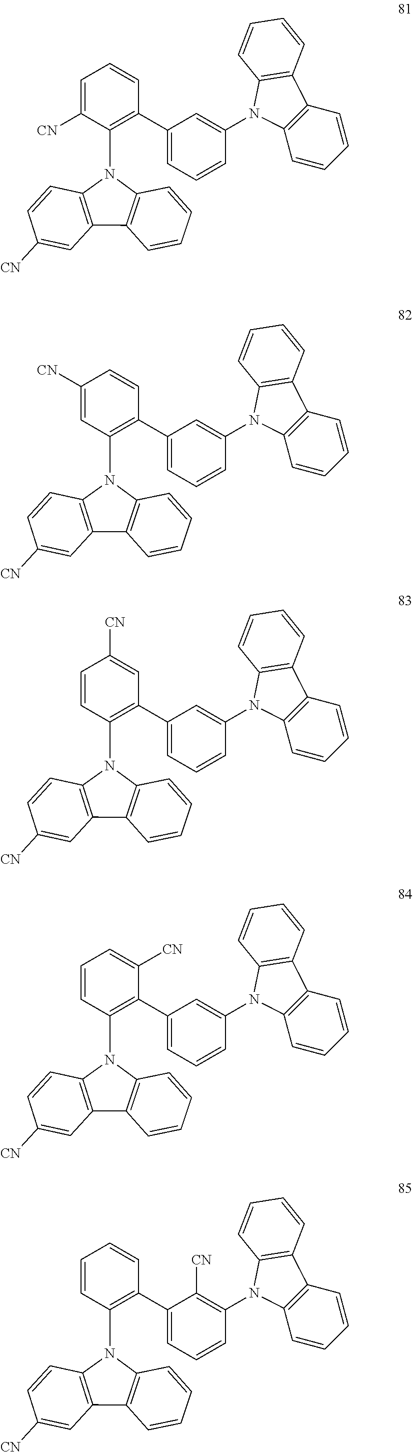

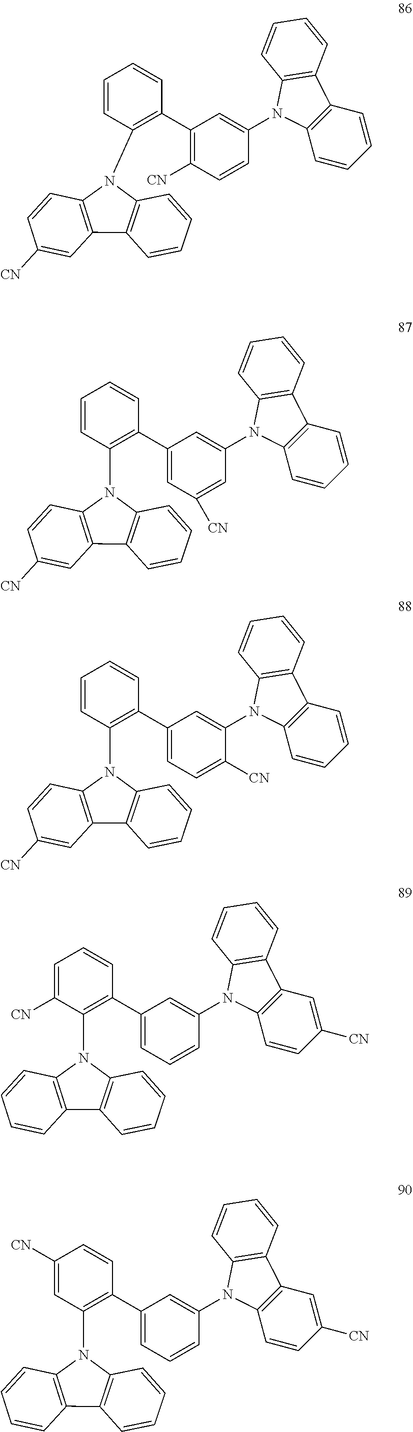

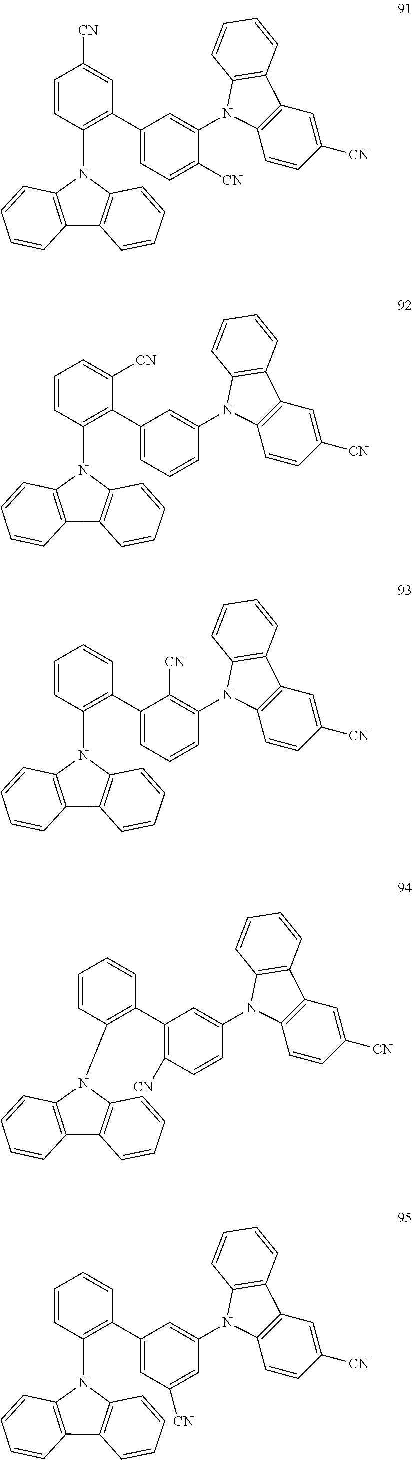

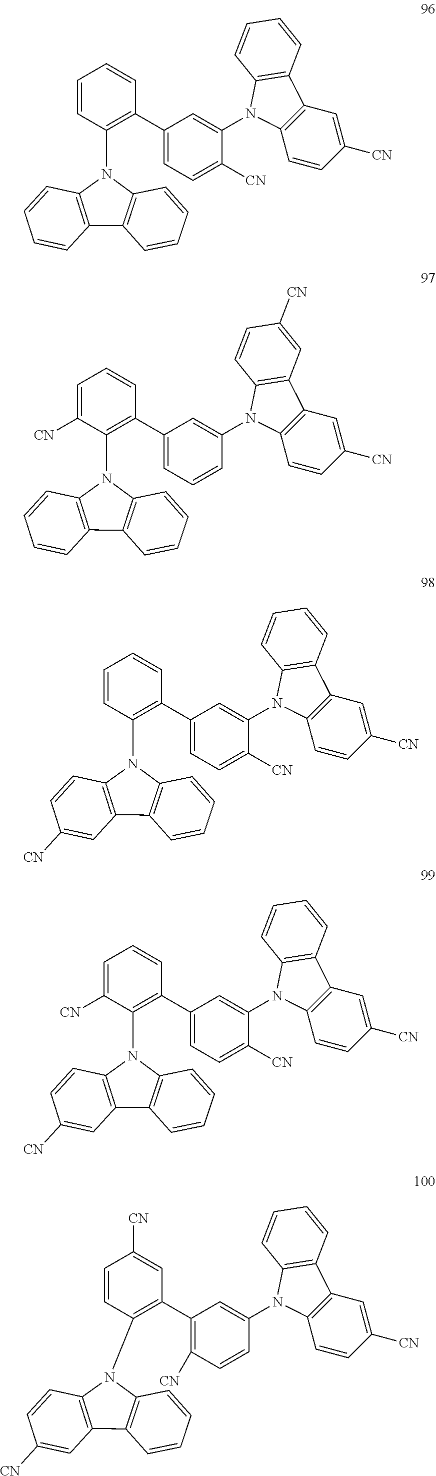

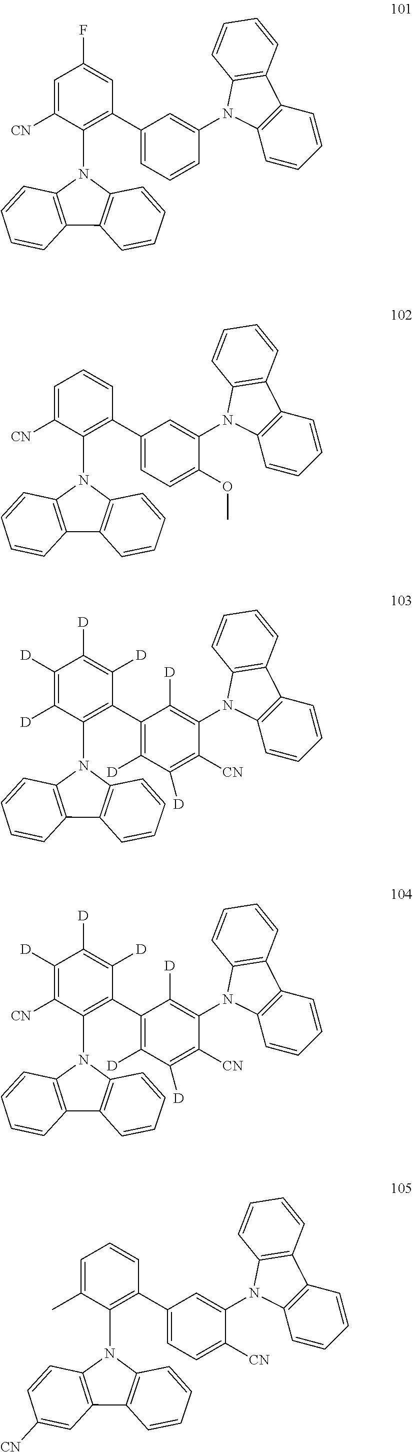

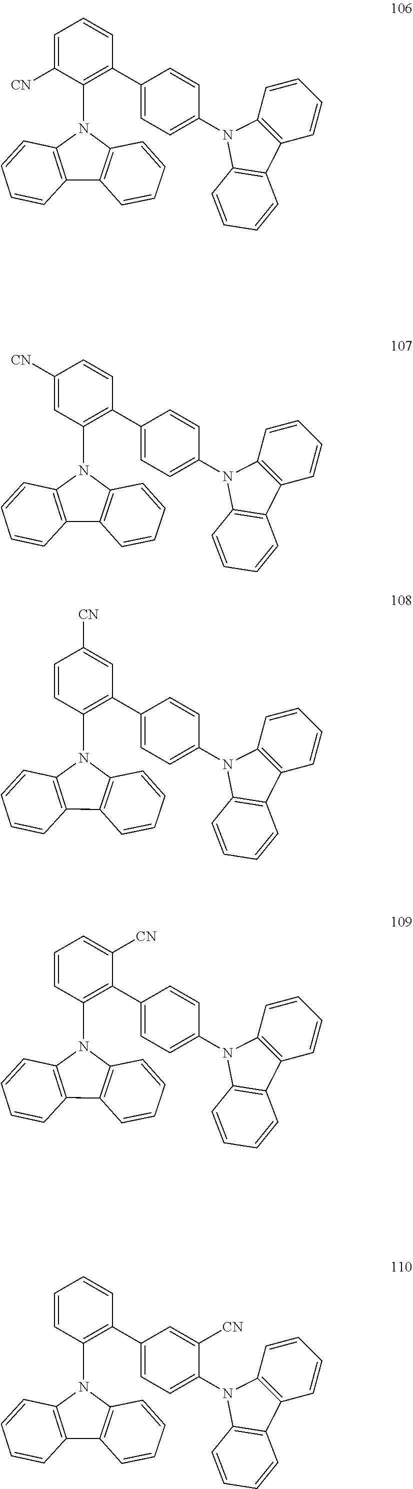

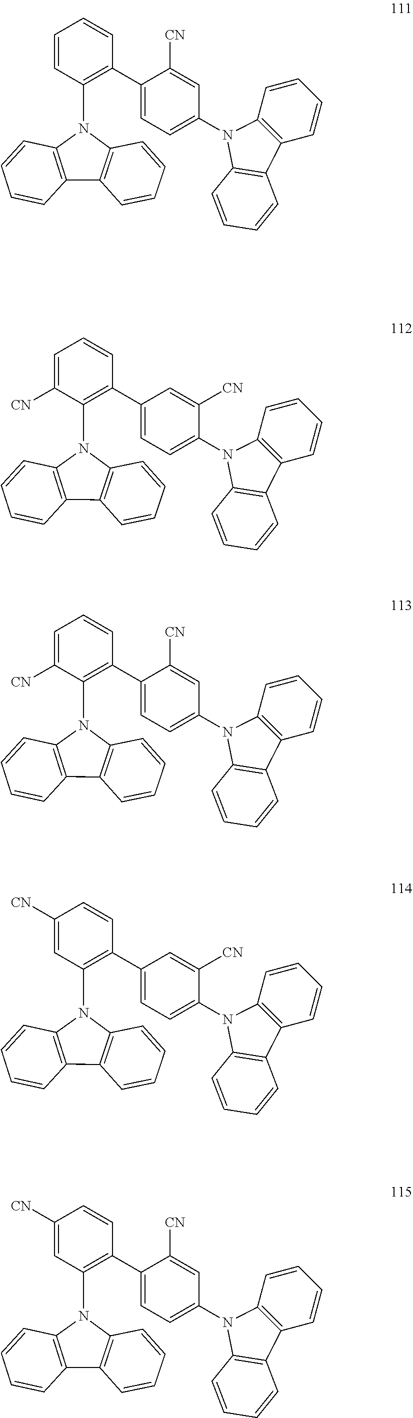

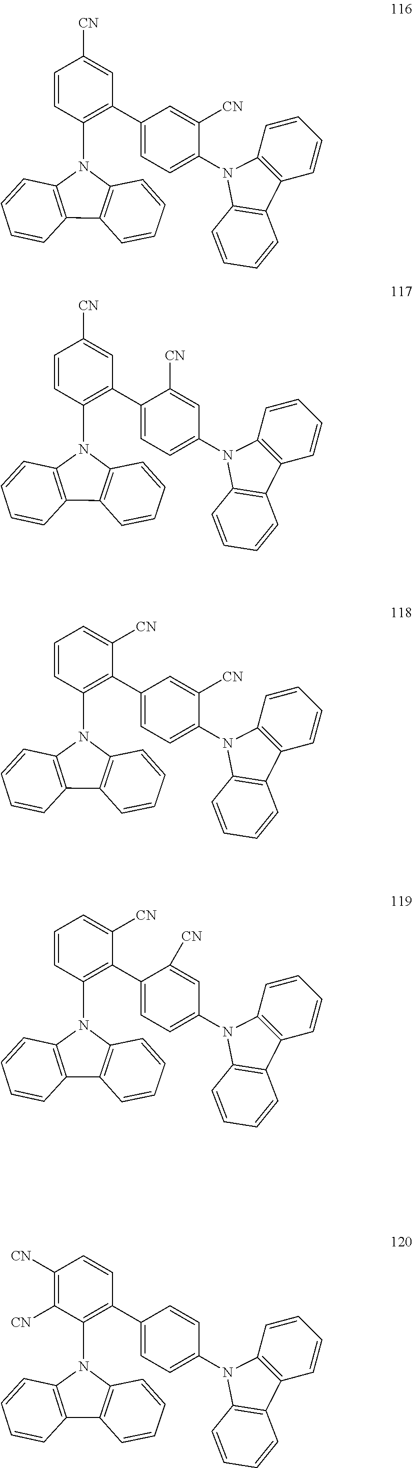

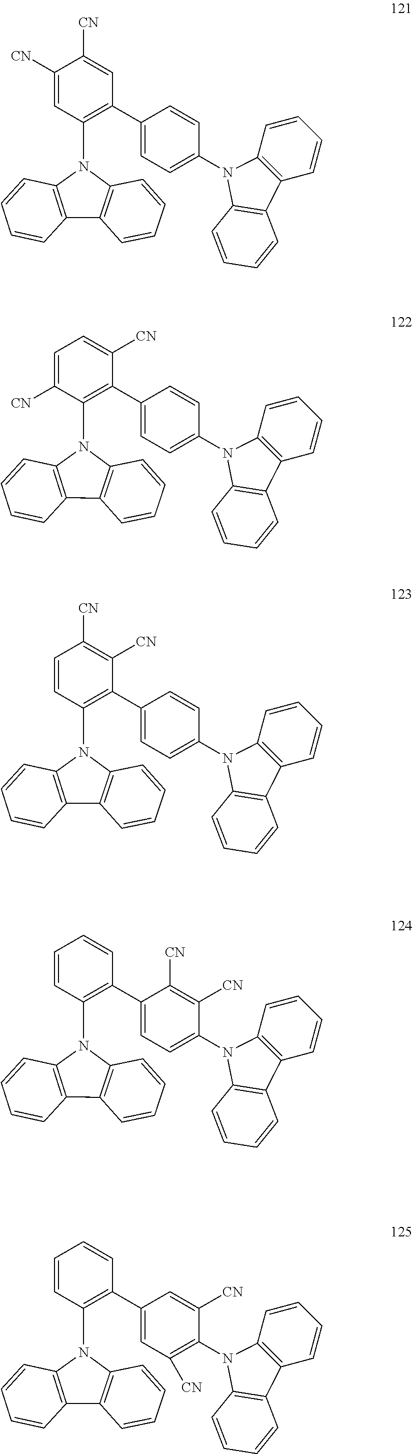

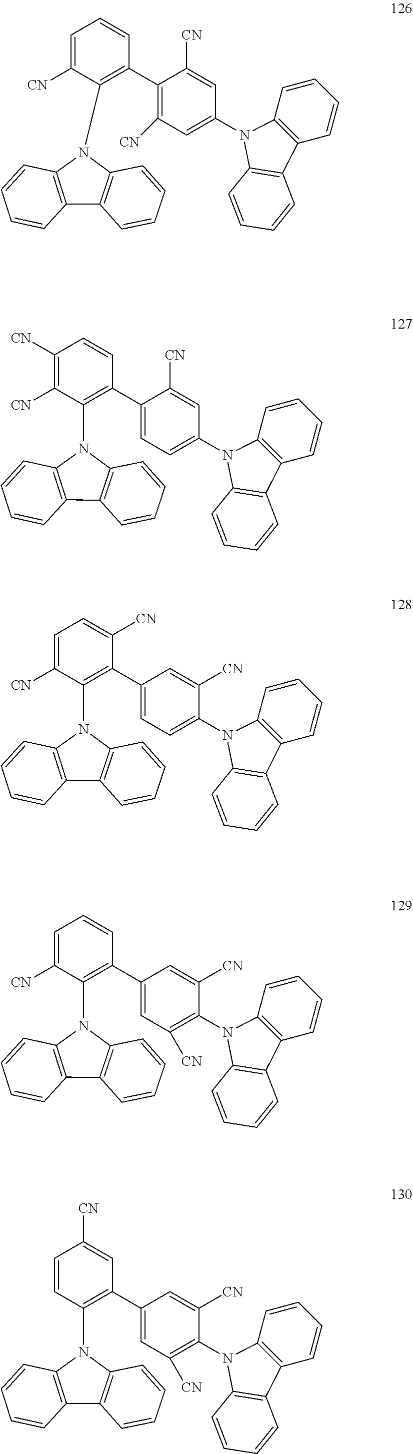

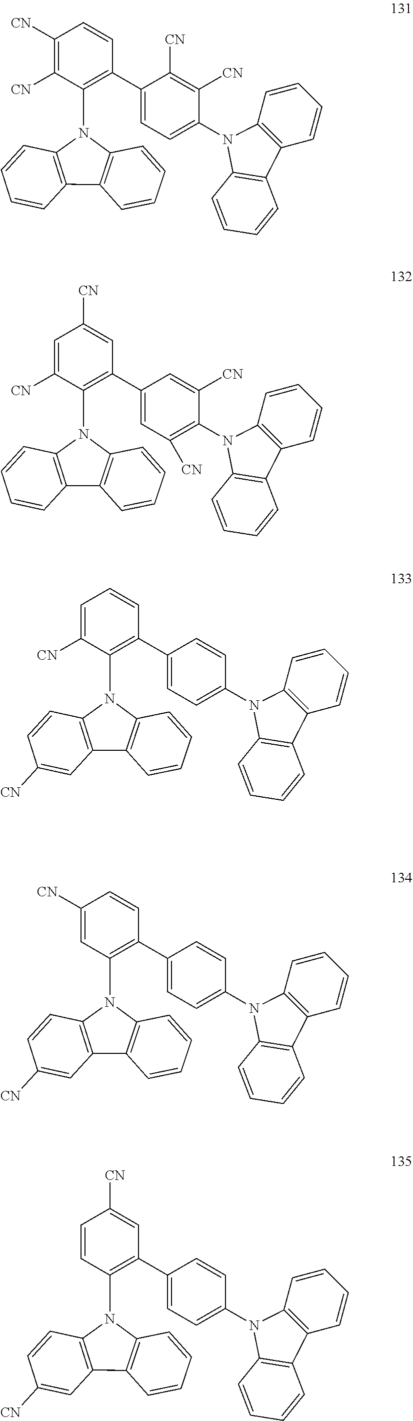

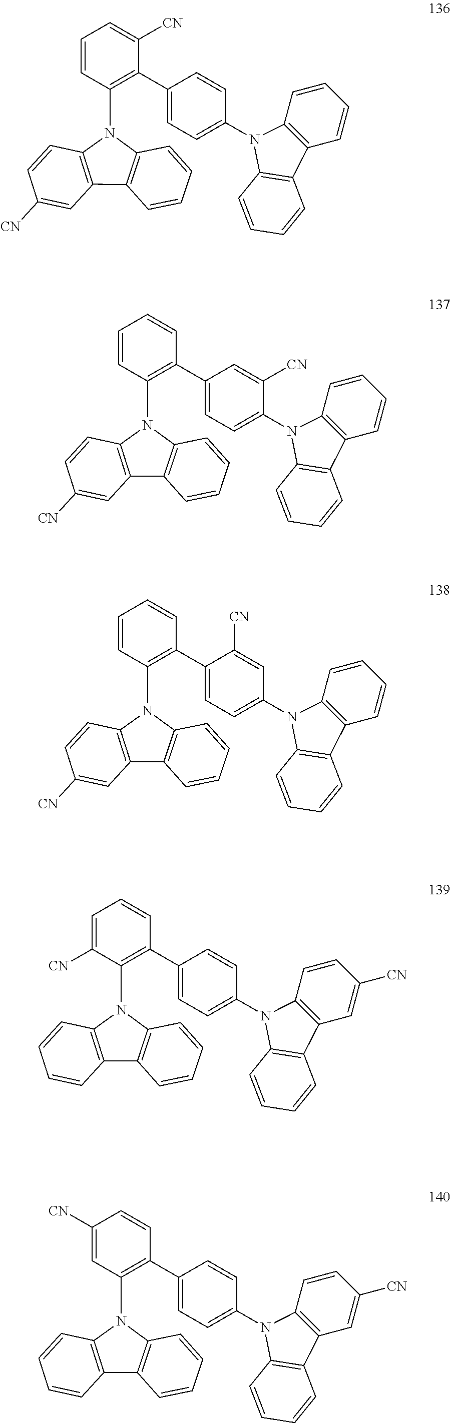

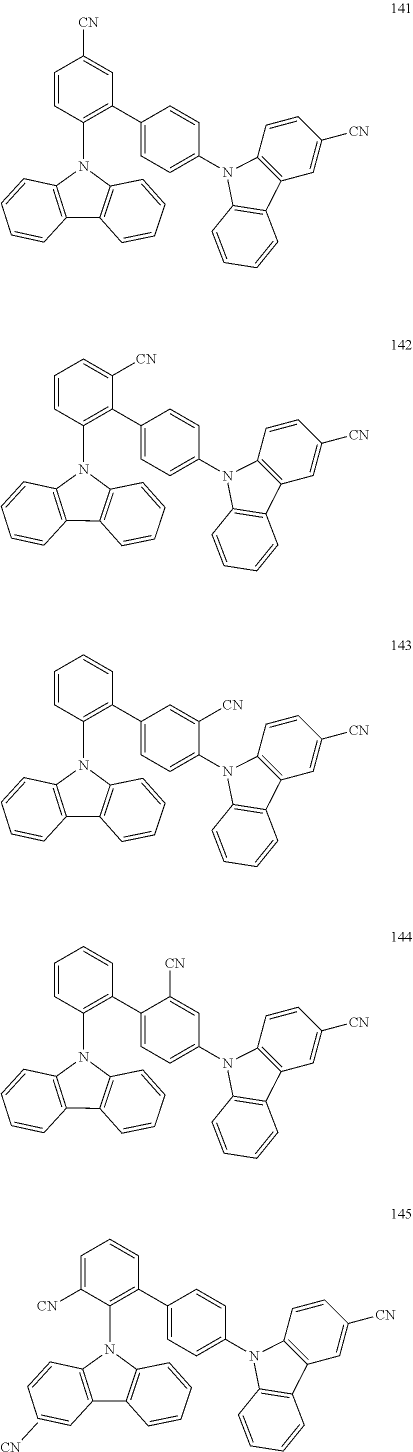

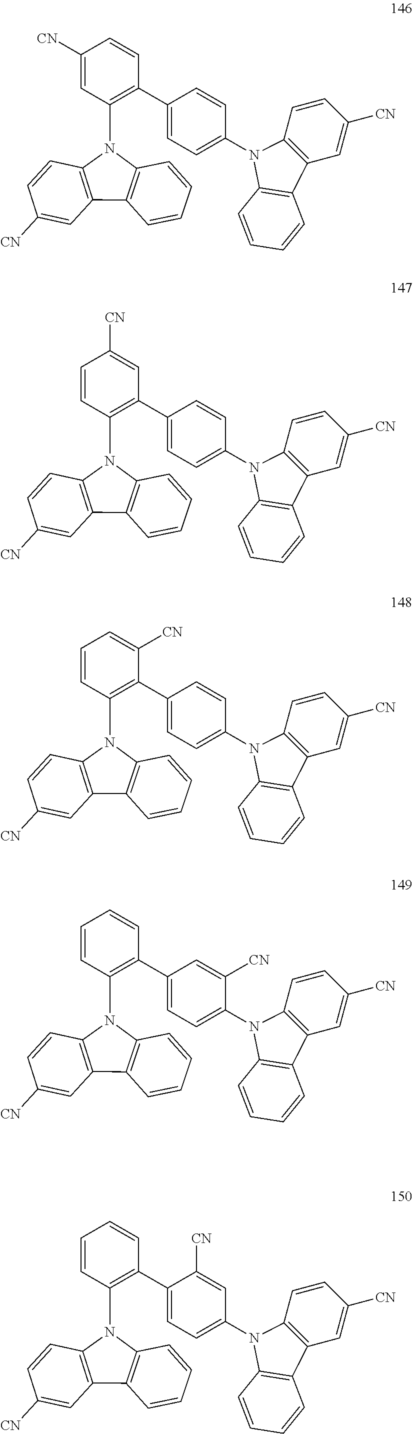

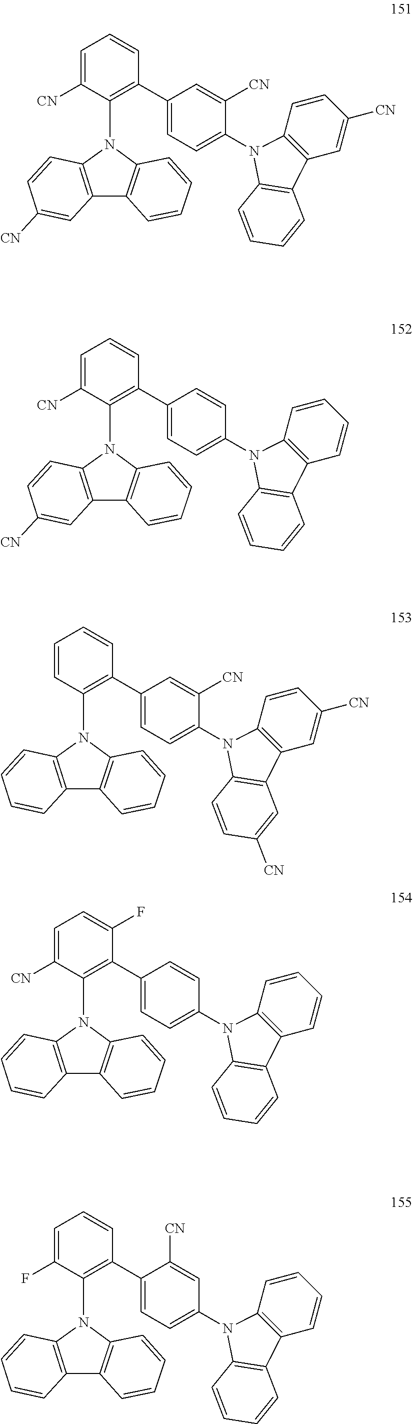

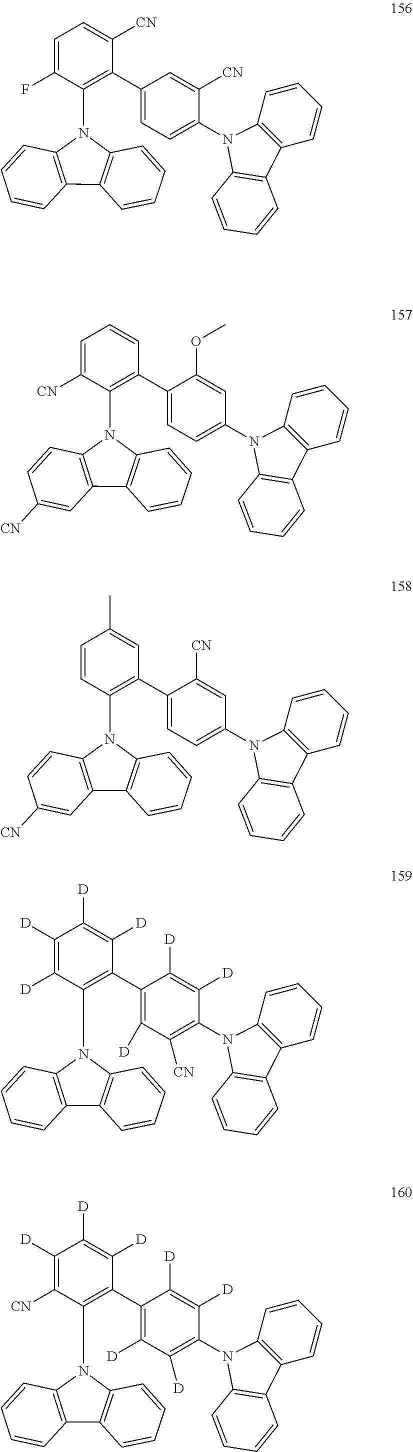

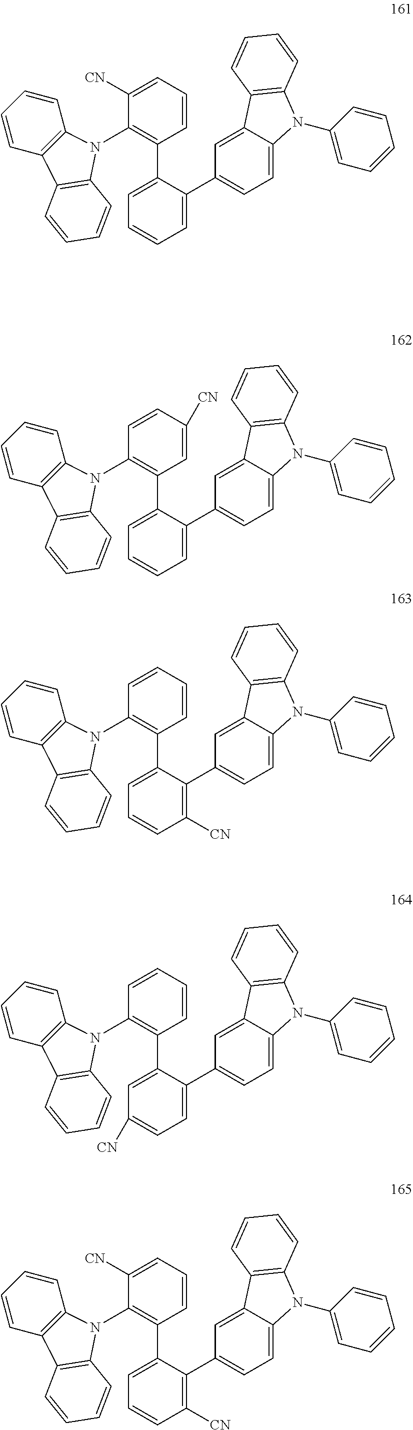

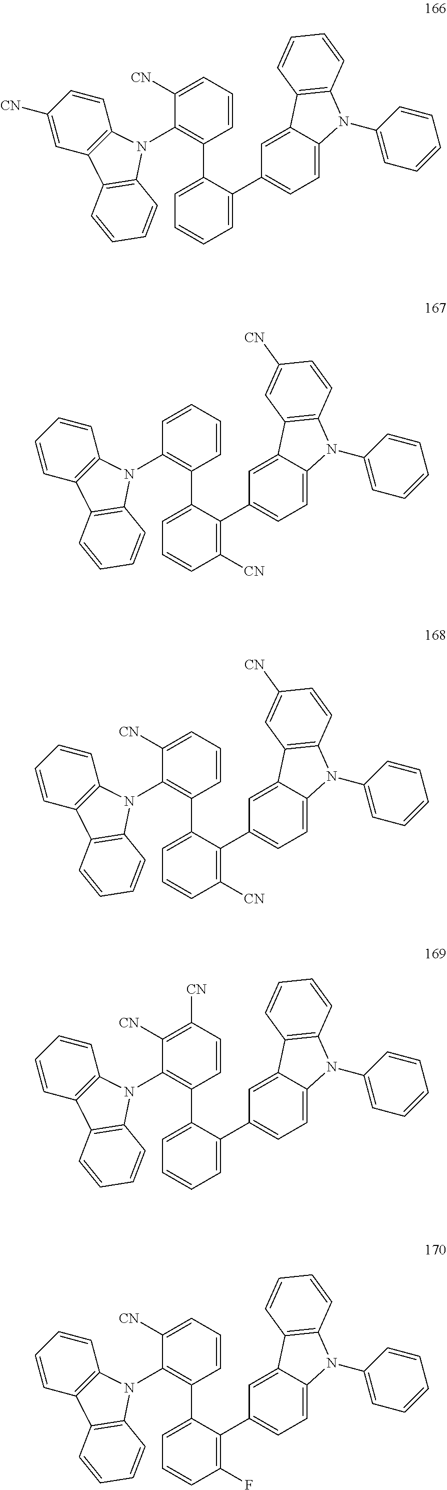

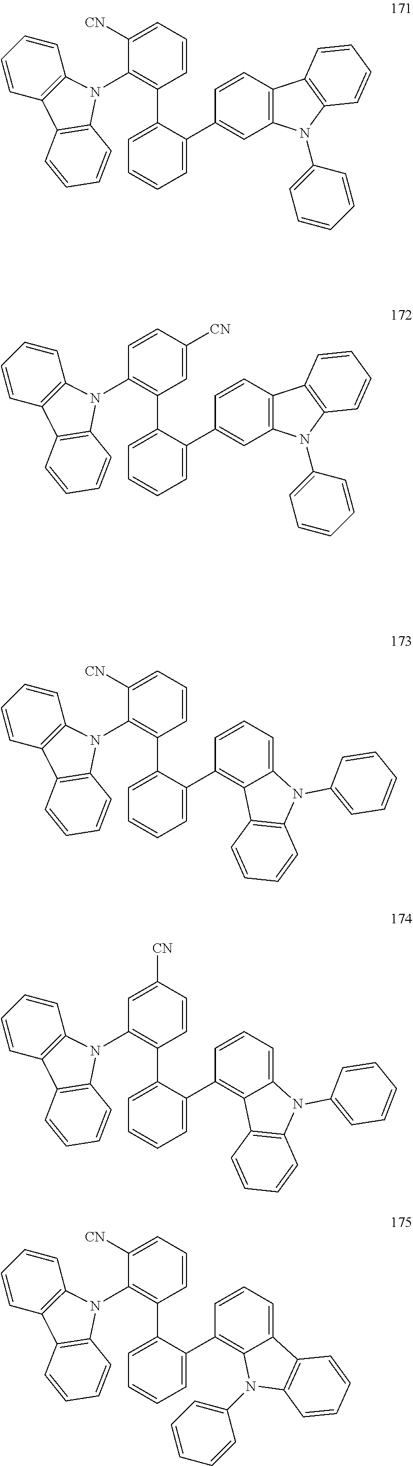

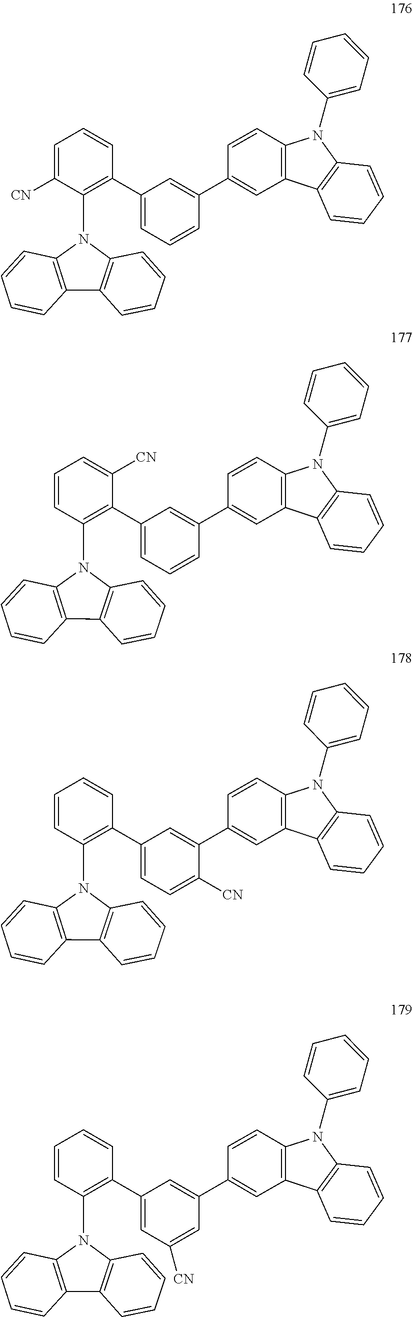

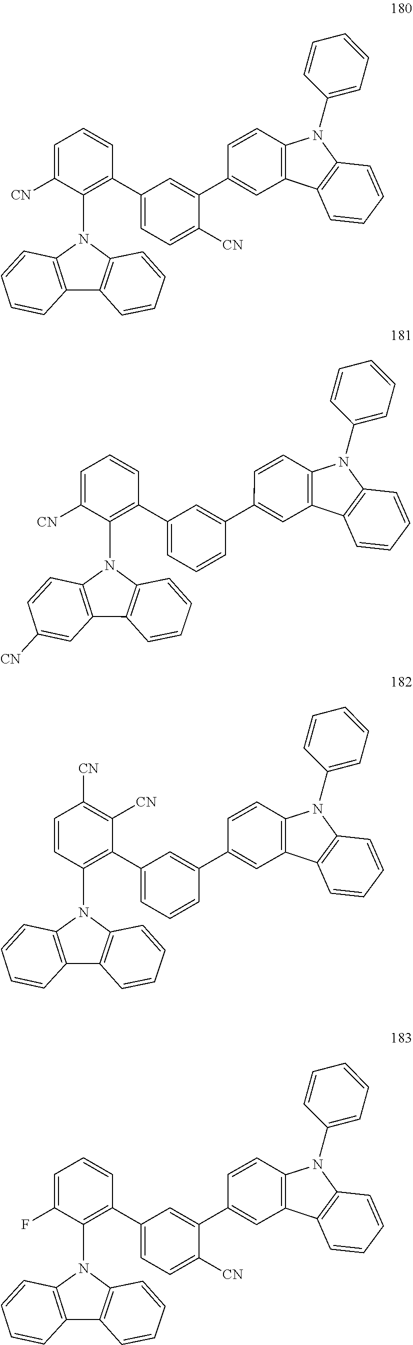

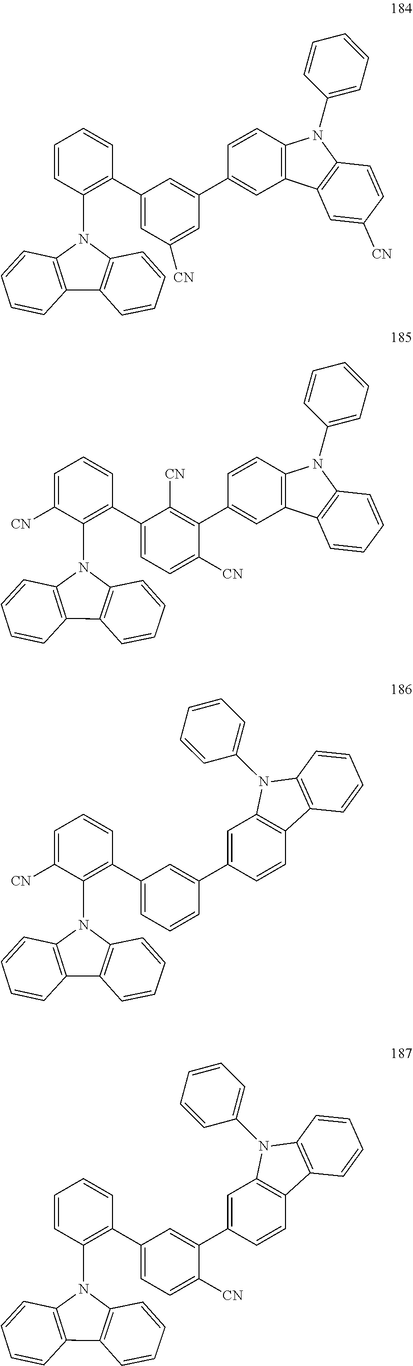

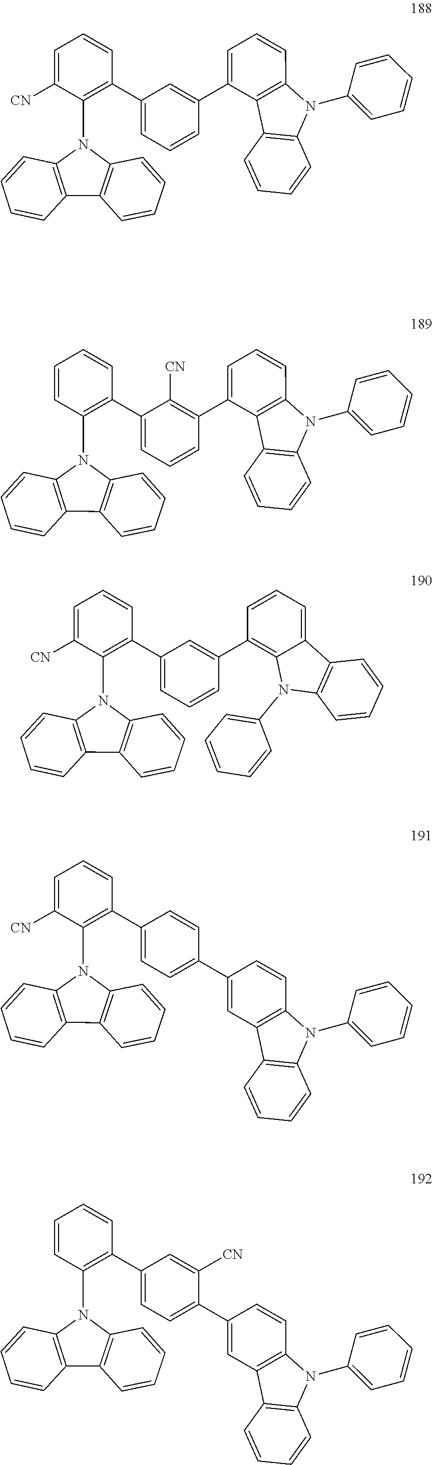

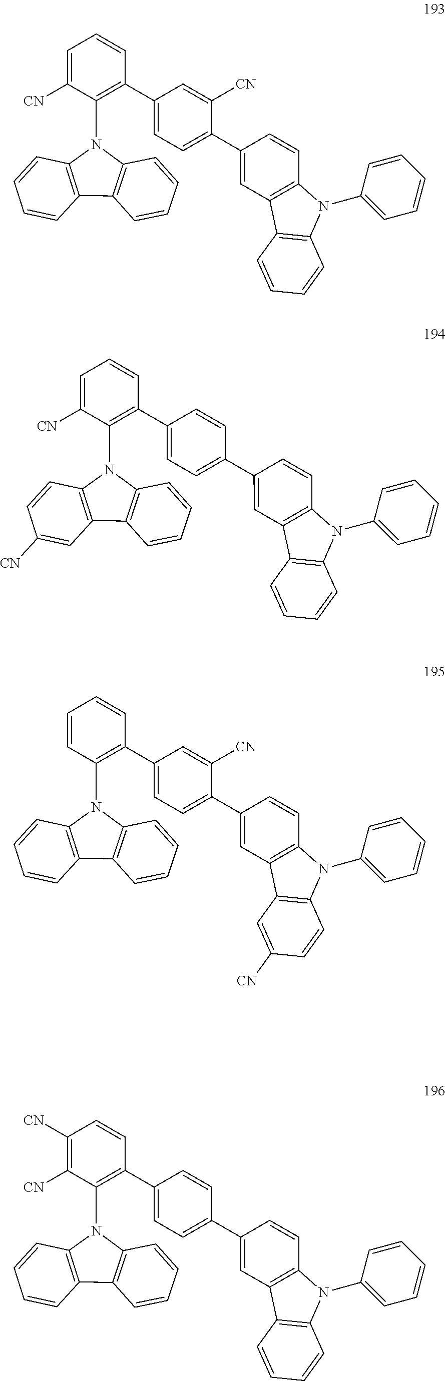

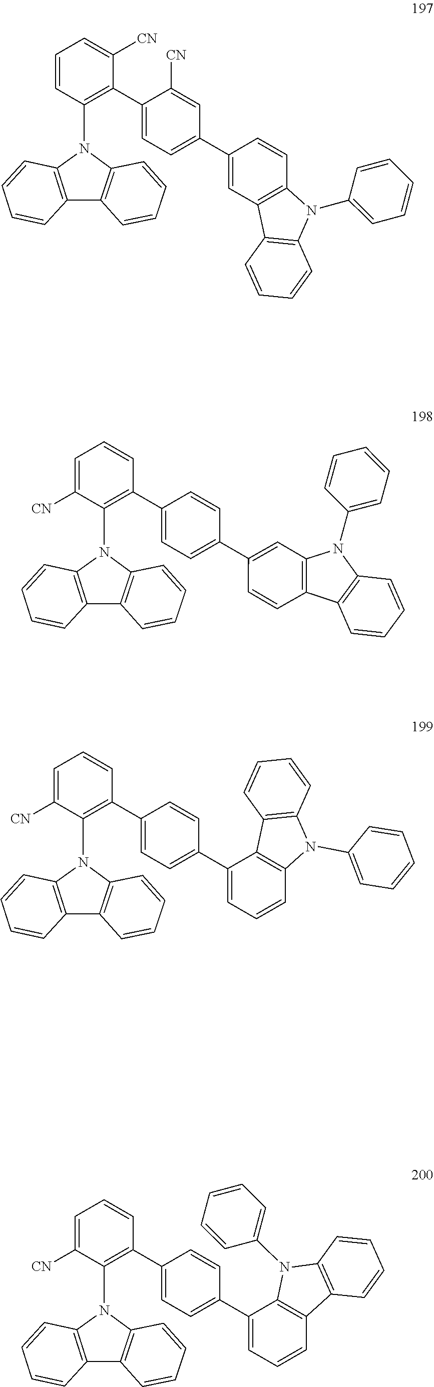

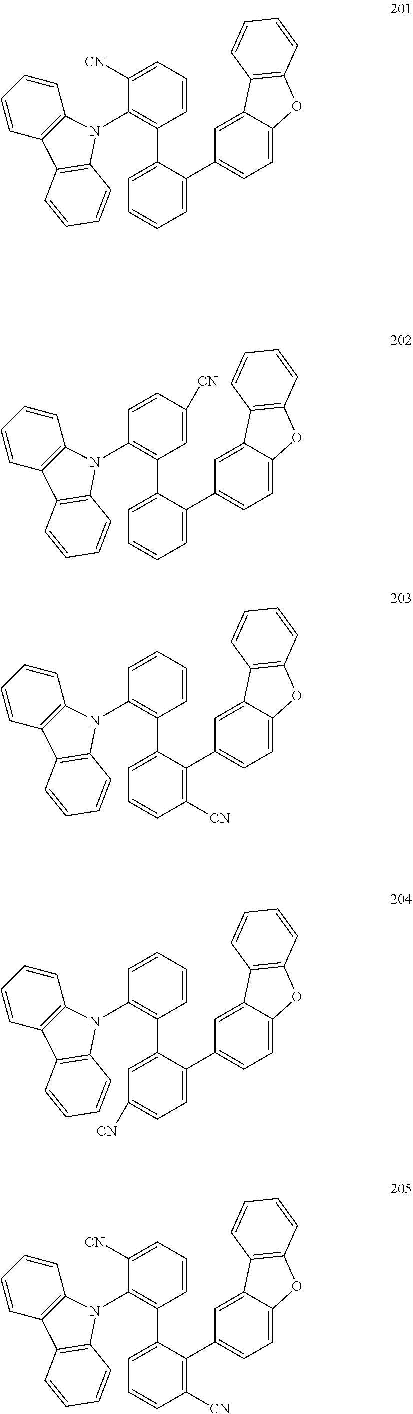

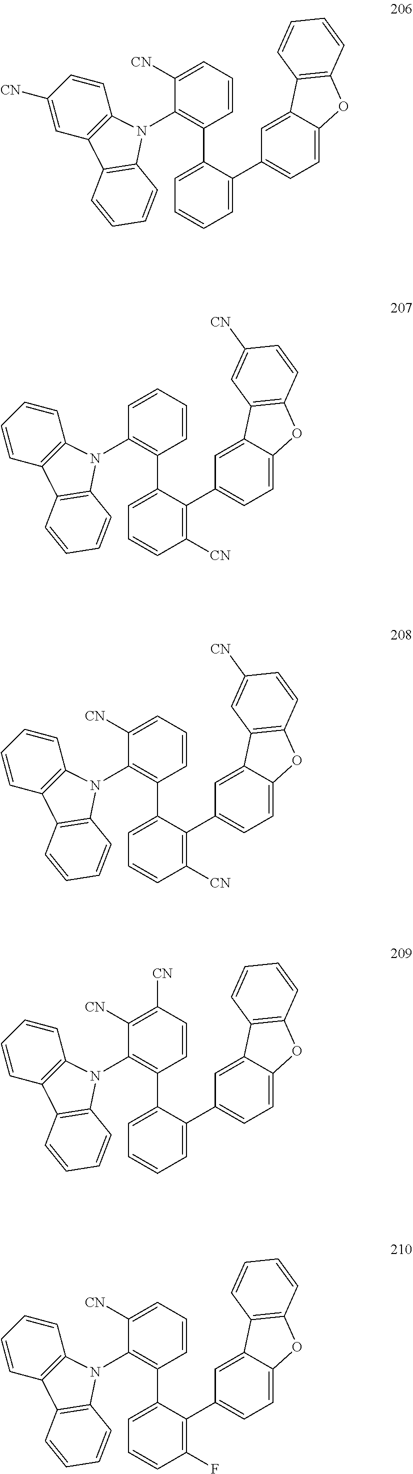

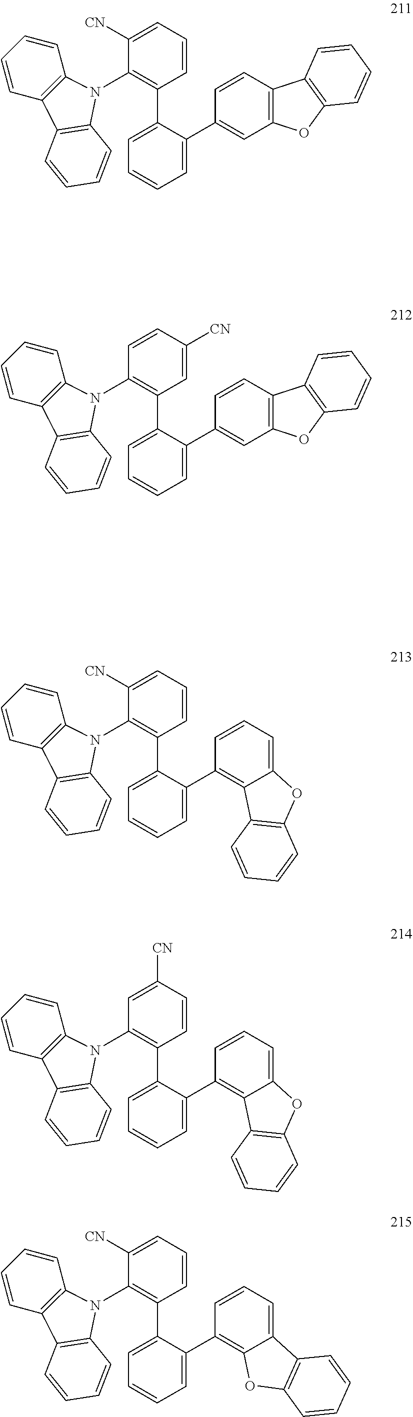

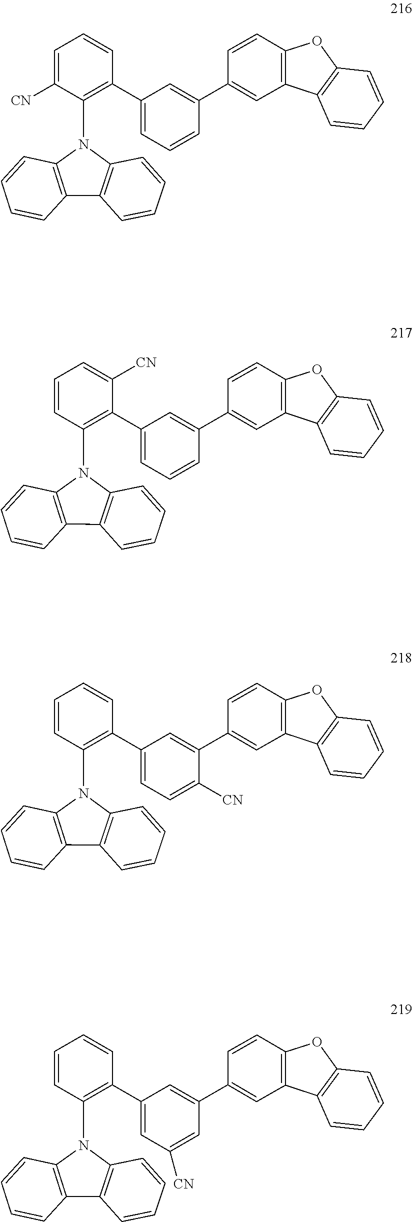

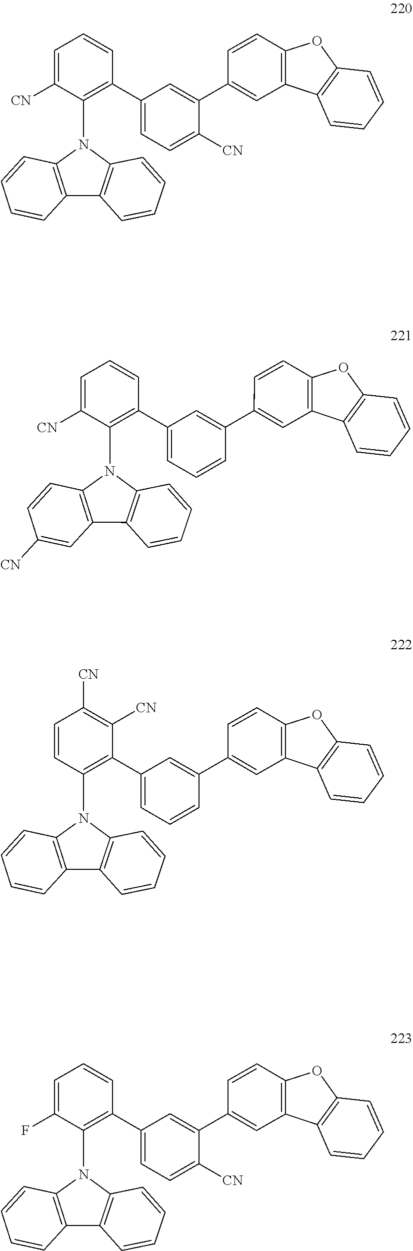

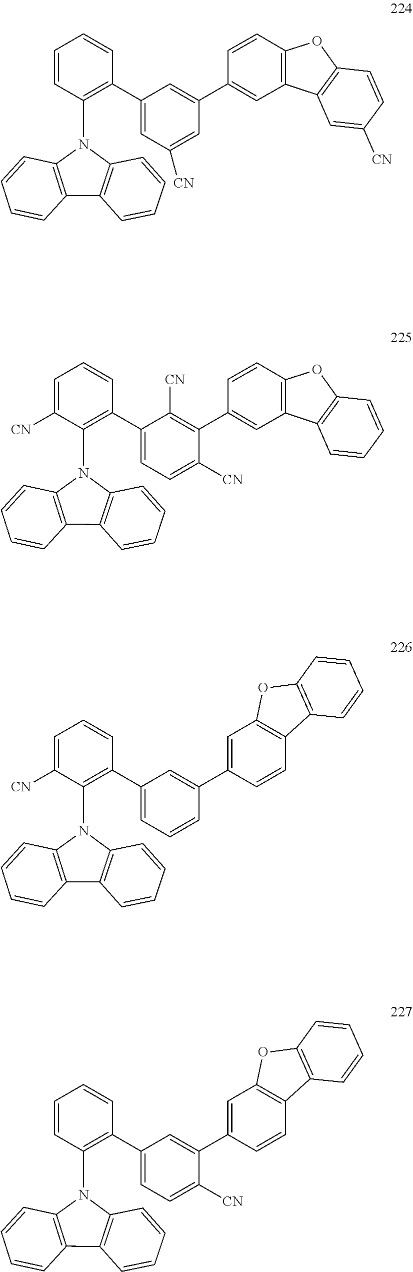

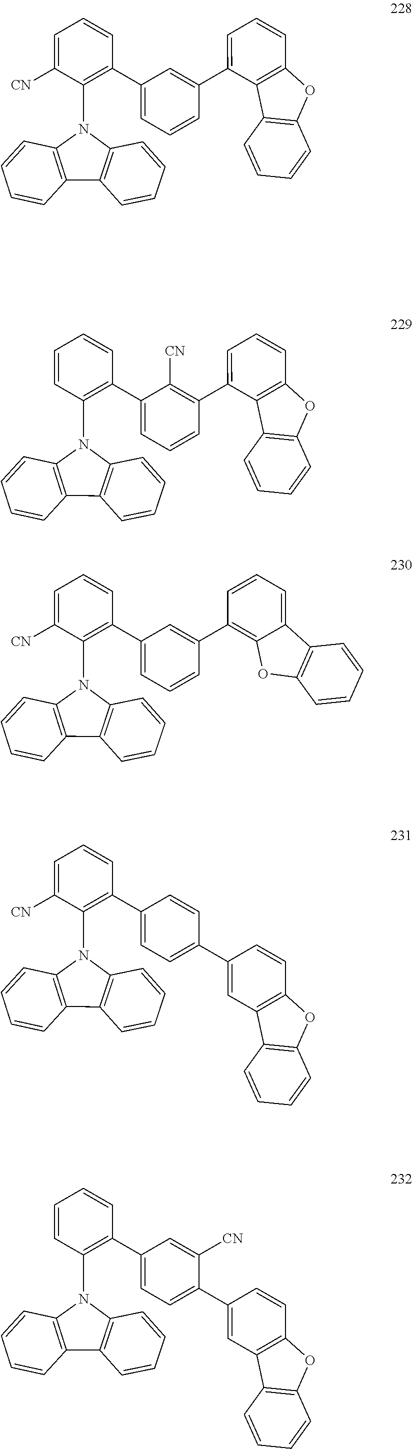

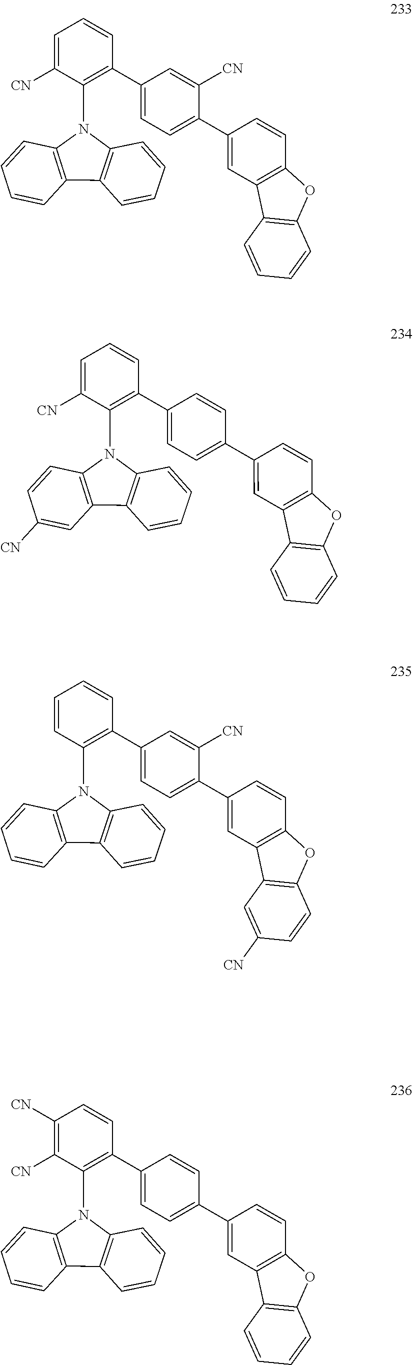

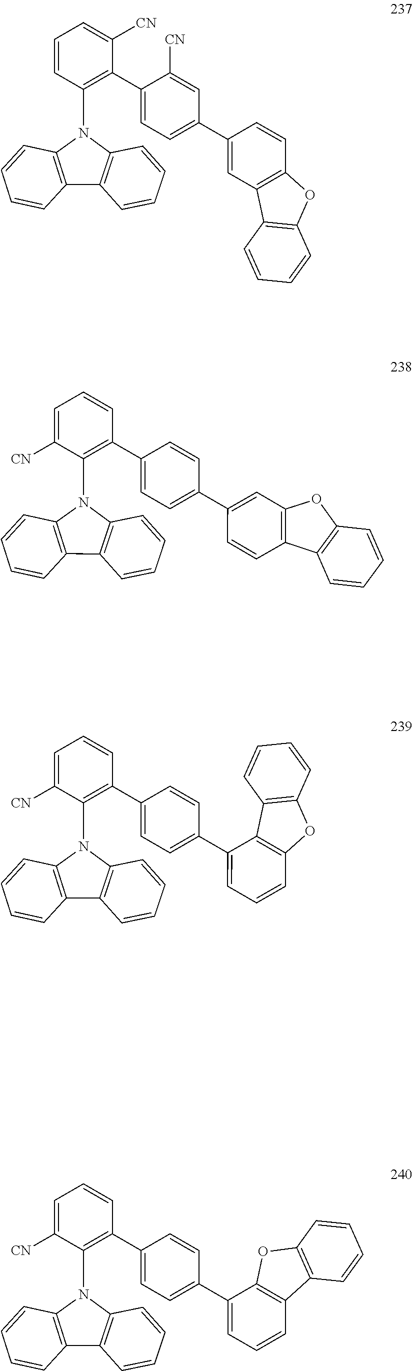

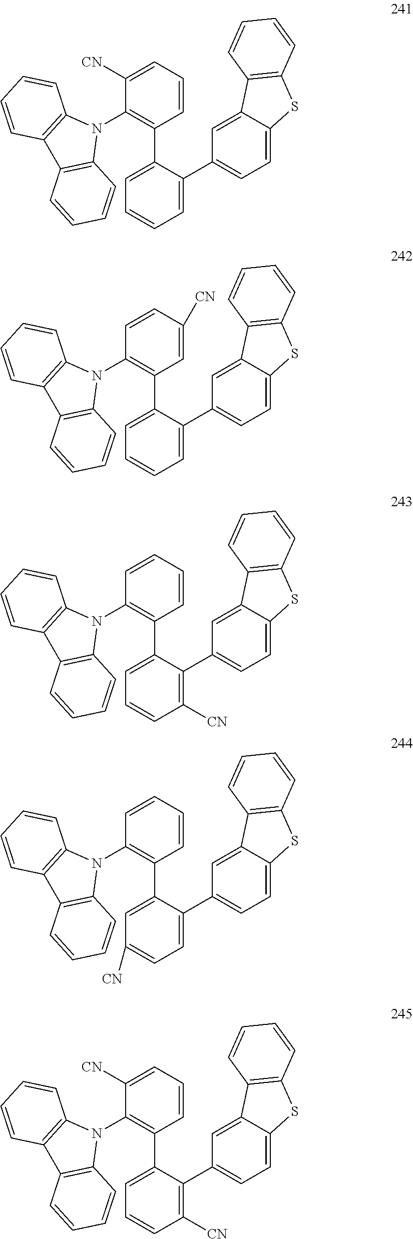

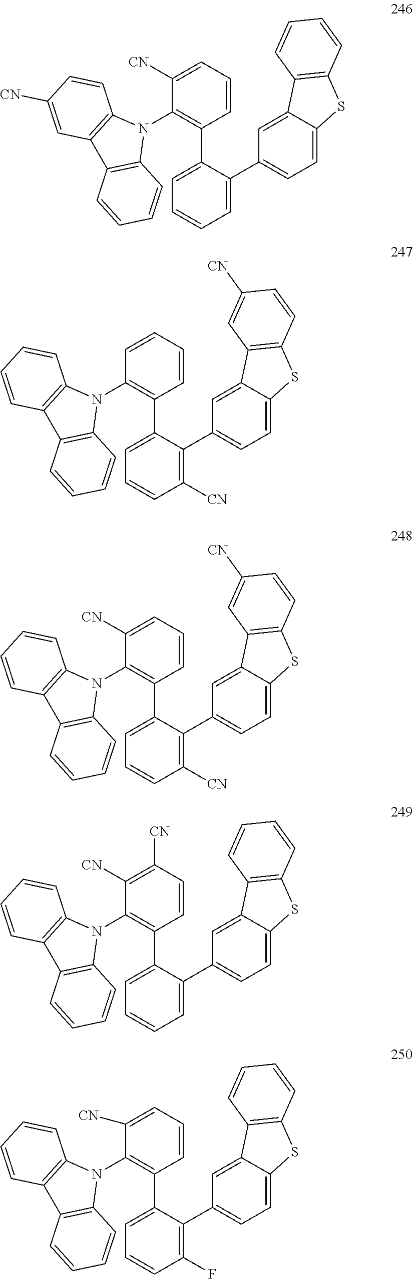

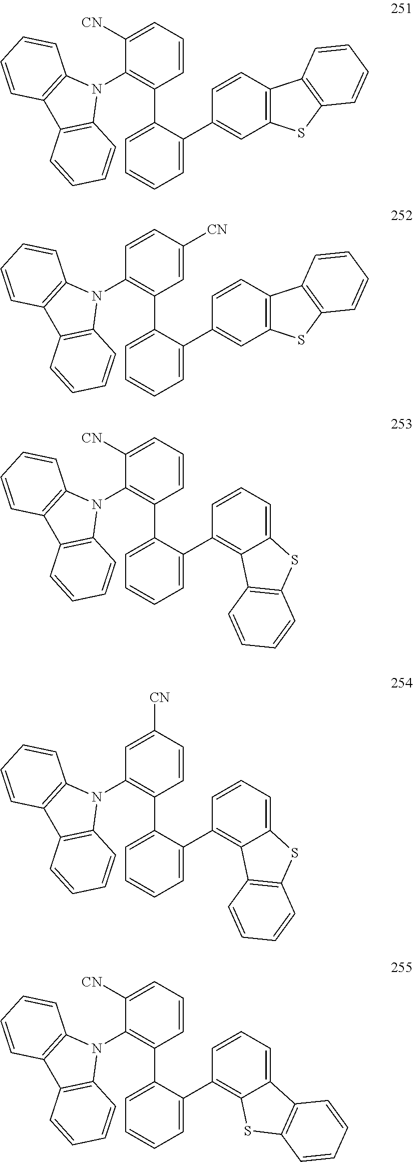

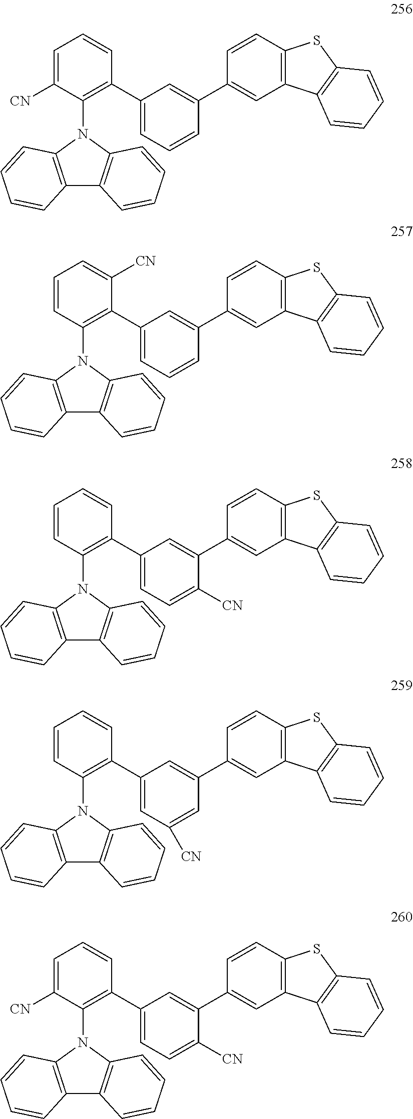

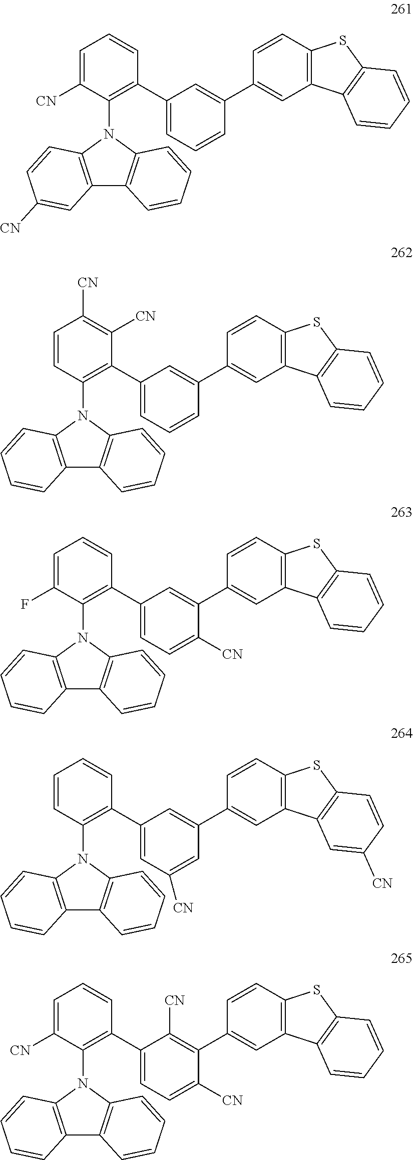

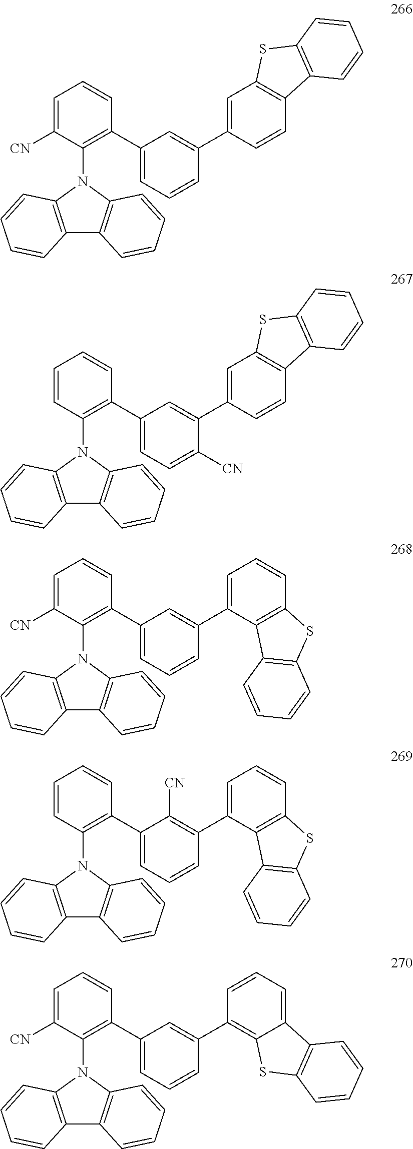

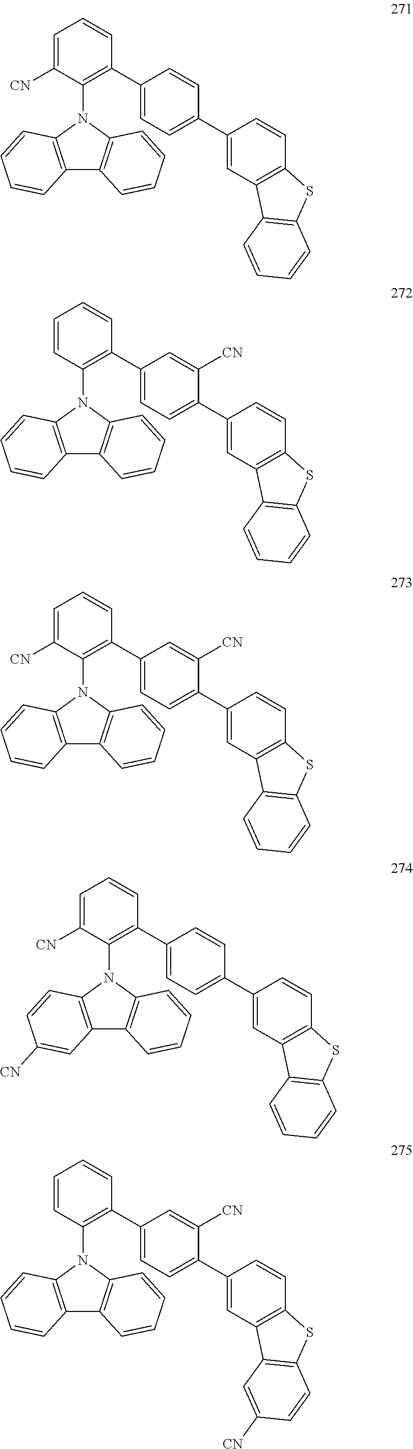

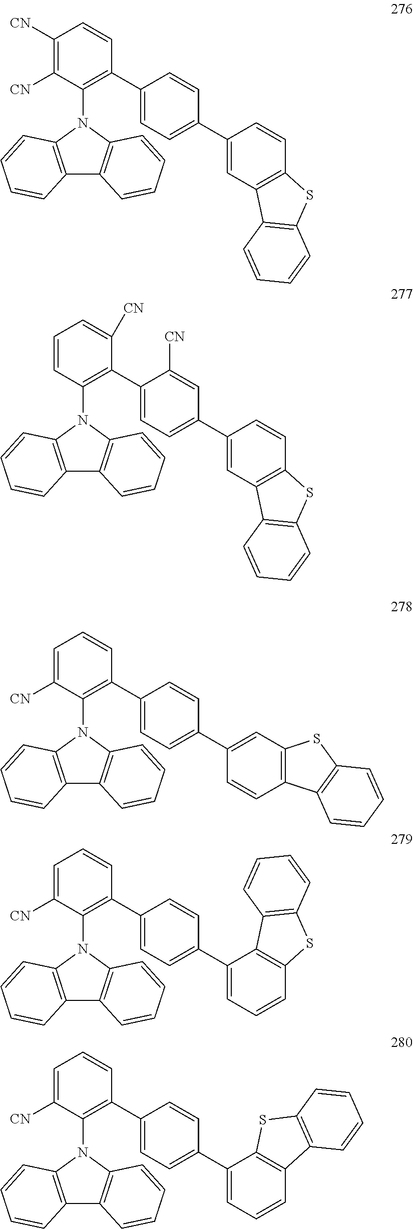

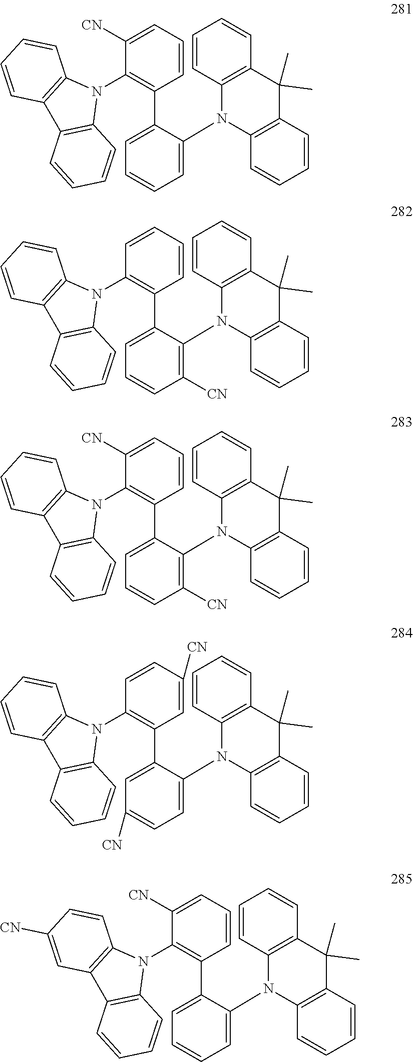

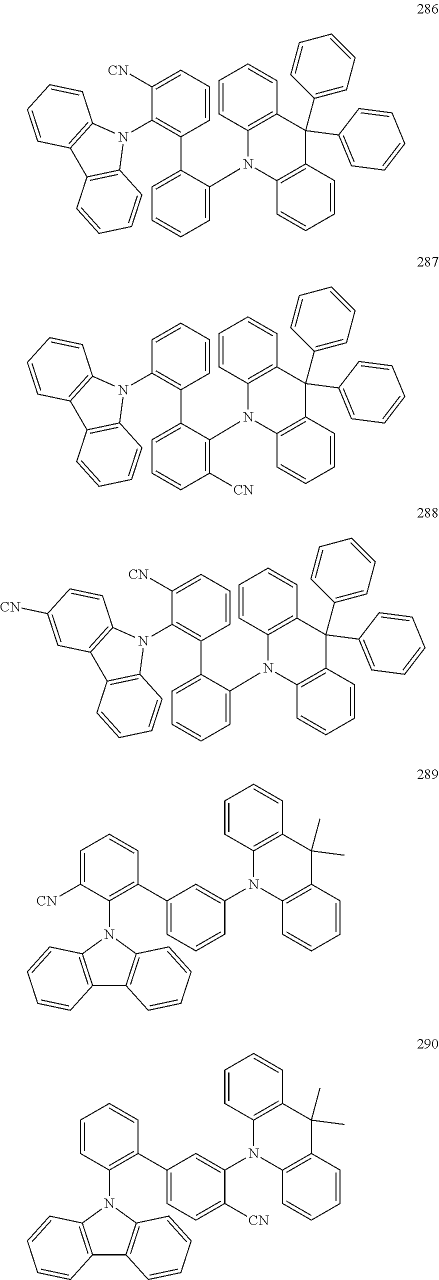

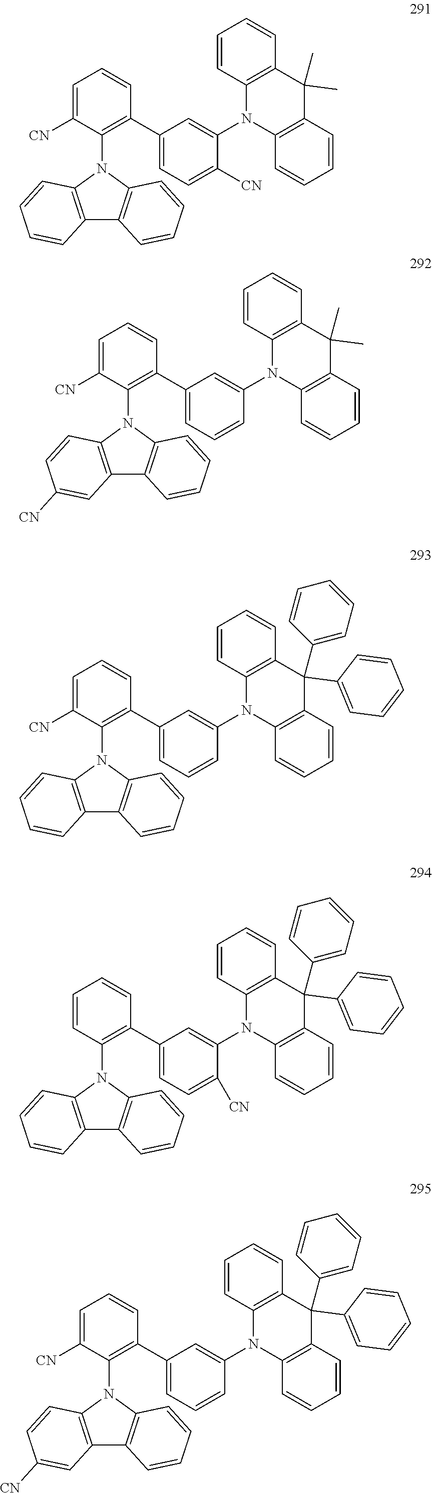

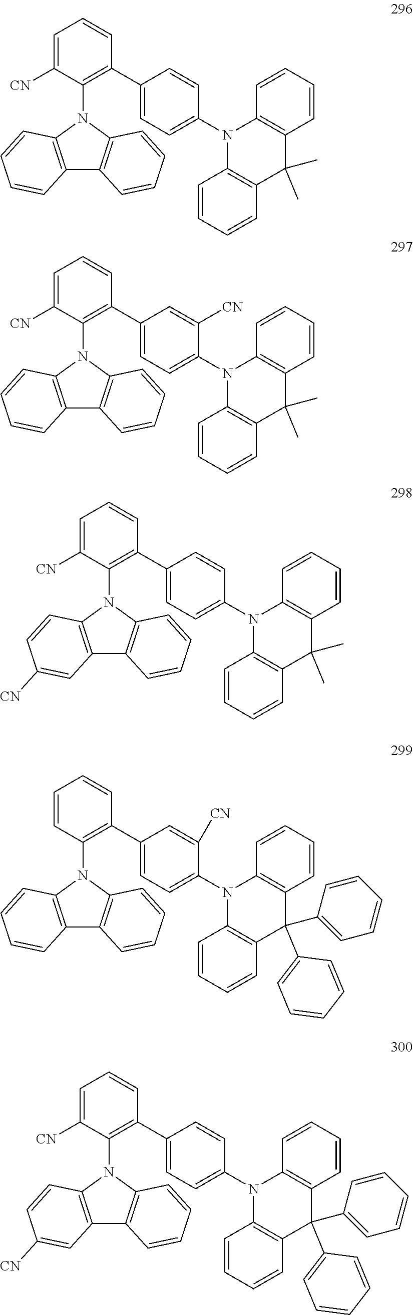

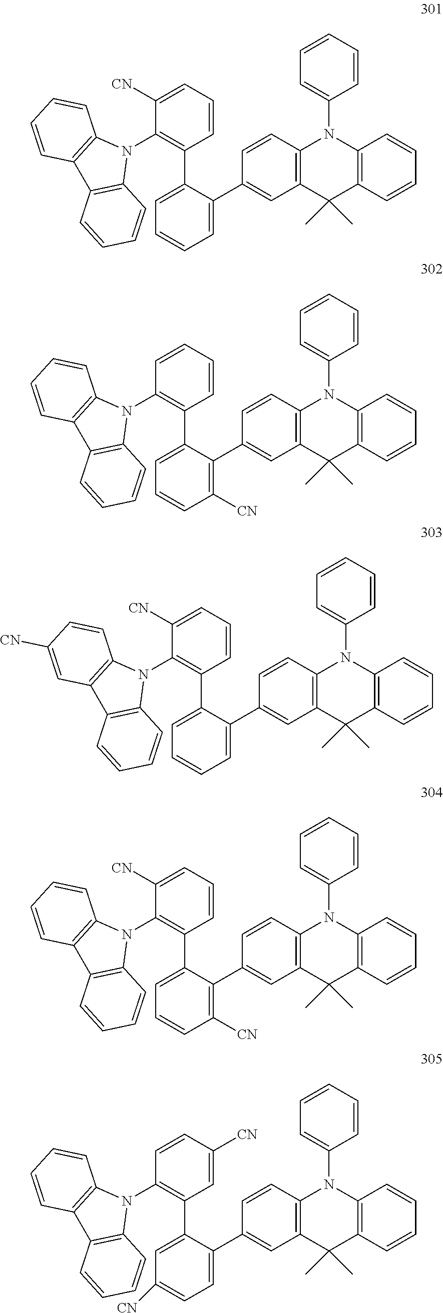

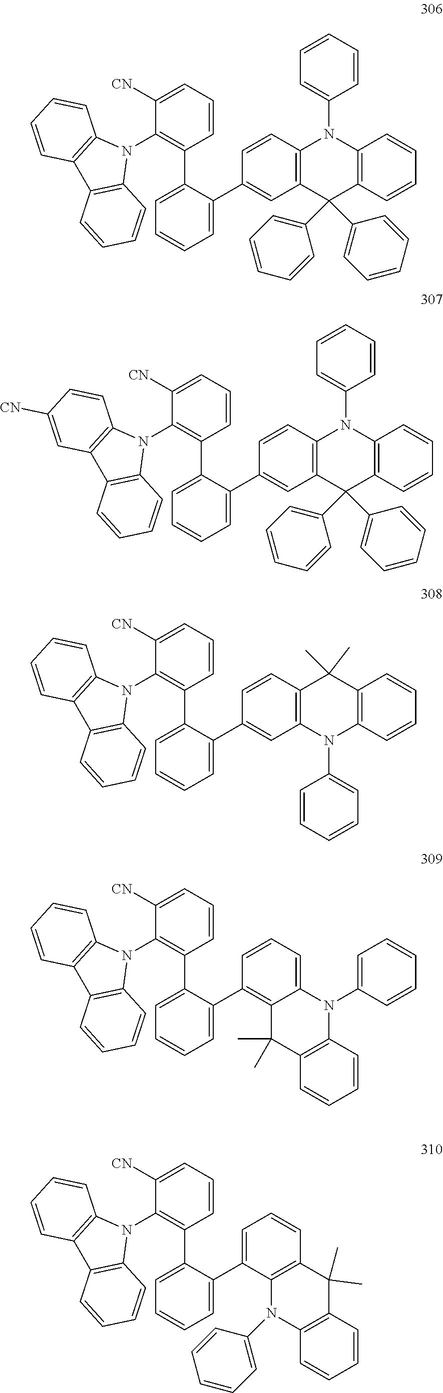

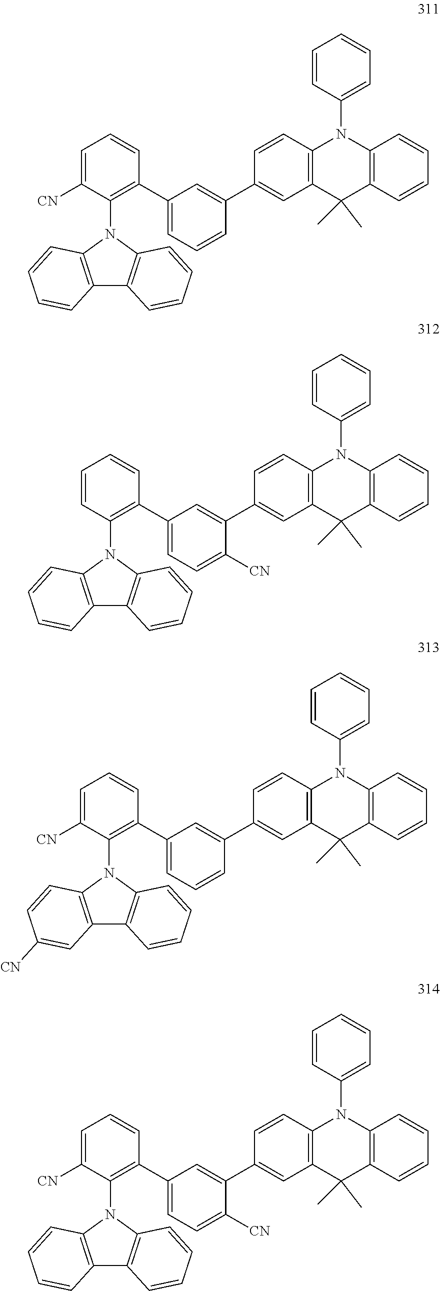

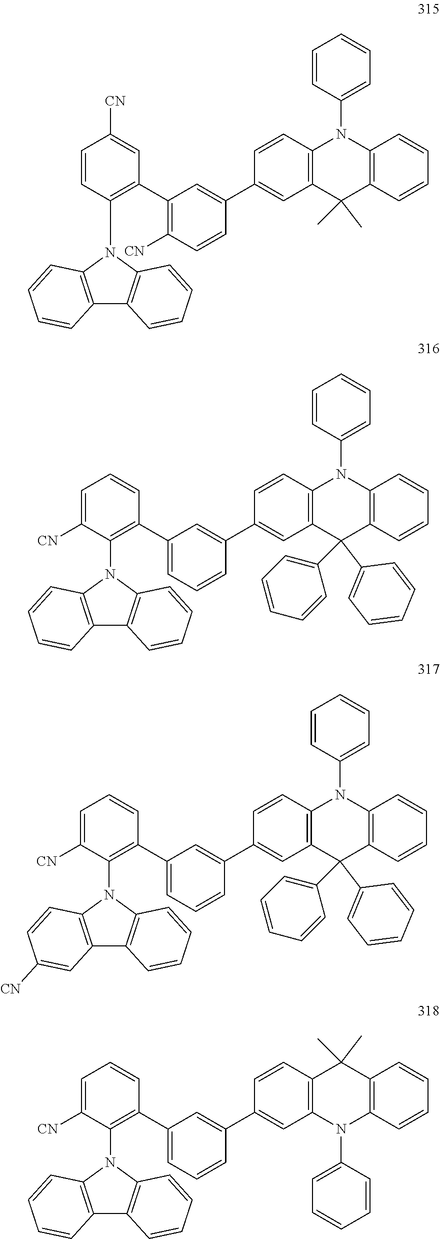

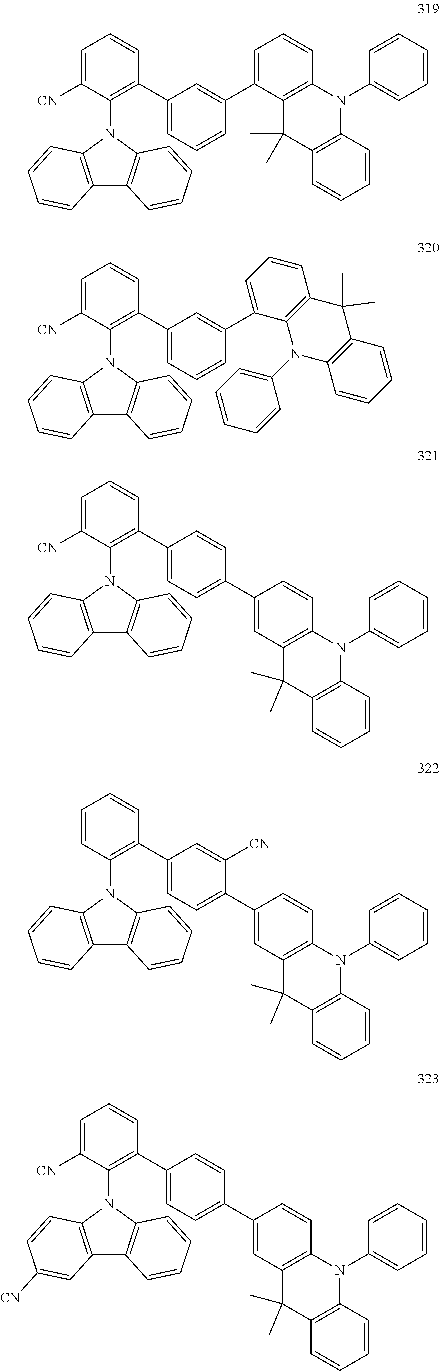

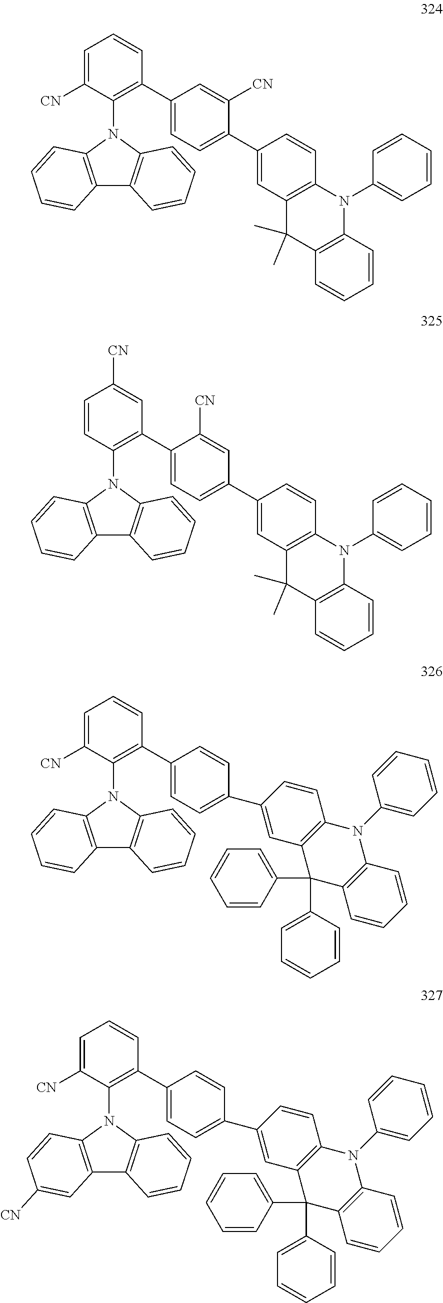

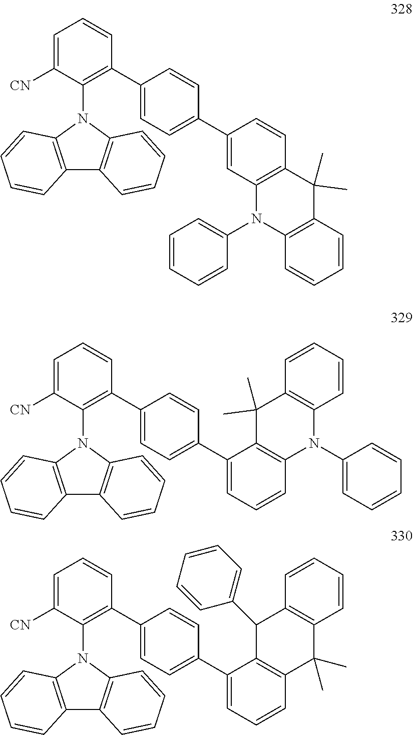

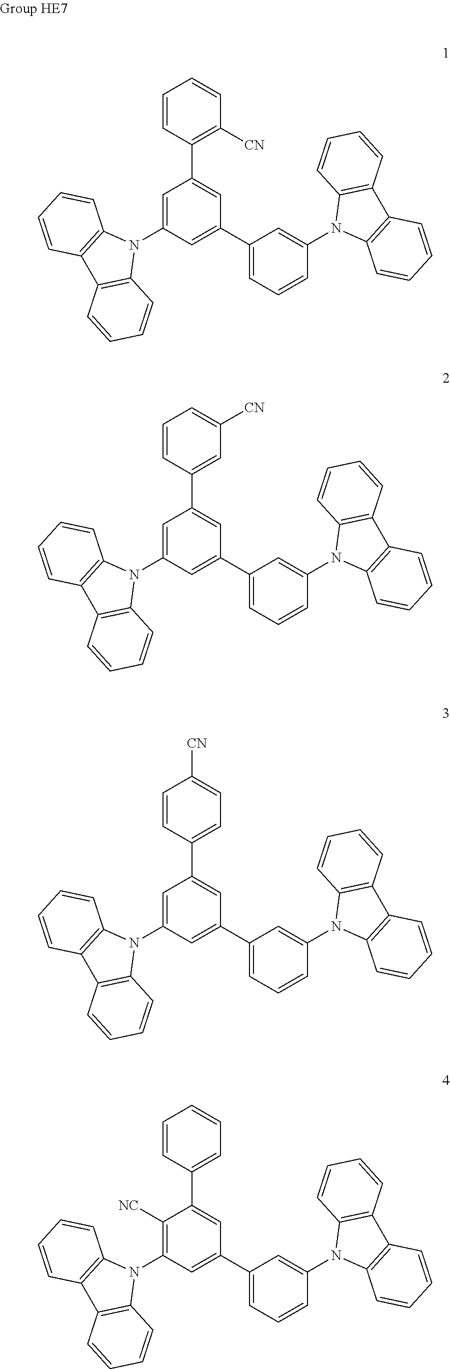 [SMILES: N#Cc1ccccc1-c1cc(-c2cccc(-n3c4ccccc4c4ccccc43)c2)cc(-n2c3ccccc3c3ccccc32)c1 ; N#Cc1cccc(-c2cc(-c3cccc(-n4c5ccccc5c5ccccc54)c3)cc(-n3c4ccccc4c4ccccc43)c2)c1 ; N#Cc1ccc(-c2cc(-c3cccc(-n4c5ccccc5c5ccccc54)c3)cc(-n3c4ccccc4c4ccccc43)c2)cc1 ; N#Cc1c(-c2ccccc2)cc(-c2cccc(-n3c4ccccc4c4ccccc43)c2)cc1-n1c2ccccc2c2ccccc21]

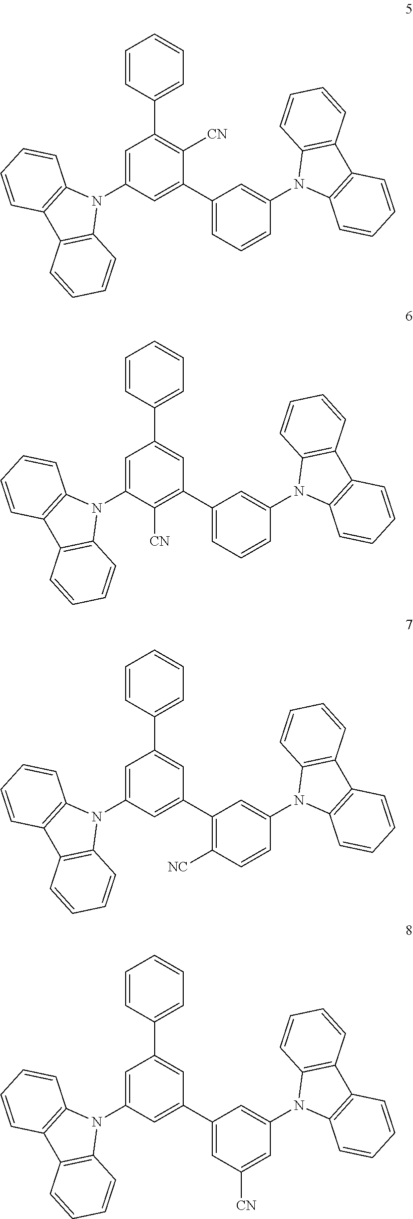 [SMILES: N#Cc1c(-c2ccccc2)cc(-n2c3ccccc3c3ccccc32)cc1-c1cccc(-n2c3ccccc3c3ccccc32)c1 ; N#Cc1c(-c2cccc(-n3c4ccccc4c4ccccc43)c2)cc(-c2ccccc2)cc1-n1c2ccccc2c2ccccc21 ; N#Cc1ccc(-n2c3ccccc3c3ccccc32)cc1-c1cc(-c2ccccc2)cc(-n2c3ccccc3c3ccccc32)c1 ; N#Cc1cc(-c2cc(-c3ccccc3)cc(-n3c4ccccc4c4ccccc43)c2)cc(-n2c3ccccc3c3ccccc32)c1]

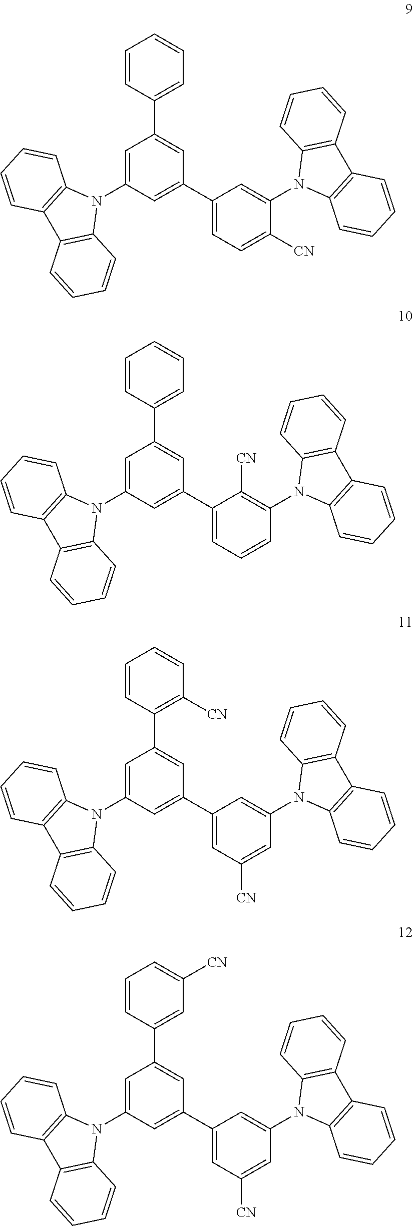 [SMILES: N#Cc1ccc(-c2cc(-c3ccccc3)cc(-n3c4ccccc4c4ccccc43)c2)cc1-n1c2ccccc2c2ccccc21 ; N#Cc1c(-c2cc(-c3ccccc3)cc(-n3c4ccccc4c4ccccc43)c2)cccc1-n1c2ccccc2c2ccccc21 ; N#Cc1cc(-c2cc(-c3ccccc3C#N)cc(-n3c4ccccc4c4ccccc43)c2)cc(-n2c3ccccc3c3ccccc32)c1 ; N#Cc1cccc(-c2cc(-c3cc(C#N)cc(-n4c5ccccc5c5ccccc54)c3)cc(-n3c4ccccc4c4ccccc43)c2)c1]

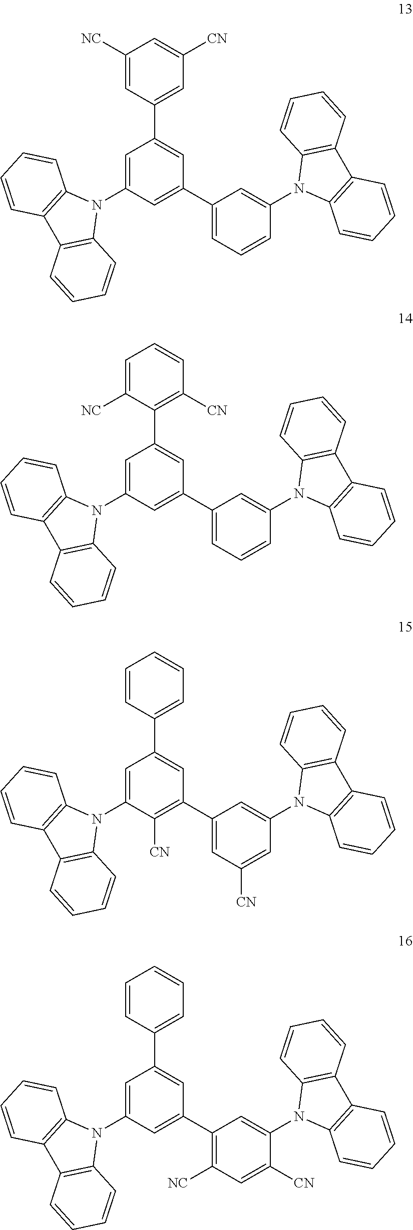 [SMILES: N#Cc1cc(C#N)cc(-c2cc(-c3cccc(-n4c5ccccc5c5ccccc54)c3)cc(-n3c4ccccc4c4ccccc43)c2)c1 ; N#Cc1cccc(C#N)c1-c1cc(-c2cccc(-n3c4ccccc4c4ccccc43)c2)cc(-n2c3ccccc3c3ccccc32)c1 ; N#Cc1cc(-c2cc(-c3ccccc3)cc(-n3c4ccccc4c4ccccc43)c2C#N)cc(-n2c3ccccc3c3ccccc32)c1 ; N#Cc1cc(C#N)c(-n2c3ccccc3c3ccccc32)cc1-c1cc(-c2ccccc2)cc(-n2c3ccccc3c3ccccc32)c1]

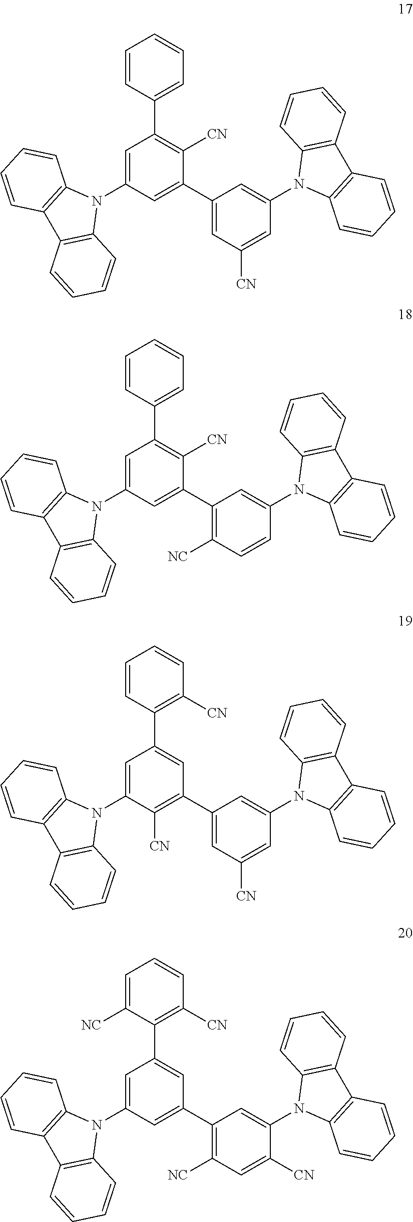 [SMILES: N#Cc1cc(-c2cc(-n3c4ccccc4c4ccccc43)cc(-c3ccccc3)c2C#N)cc(-n2c3ccccc3c3ccccc32)c1 ; N#Cc1ccc(-n2c3ccccc3c3ccccc32)cc1-c1cc(-n2c3ccccc3c3ccccc32)cc(-c2ccccc2)c1C#N ; N#Cc1cc(-c2cc(-c3ccccc3C#N)cc(-n3c4ccccc4c4ccccc43)c2C#N)cc(-n2c3ccccc3c3ccccc32)c1 ; N#Cc1cc(C#N)c(-n2c3ccccc3c3ccccc32)cc1-c1cc(-c2c(C#N)cccc2C#N)cc(-n2c3ccccc3c3ccccc32)c1]

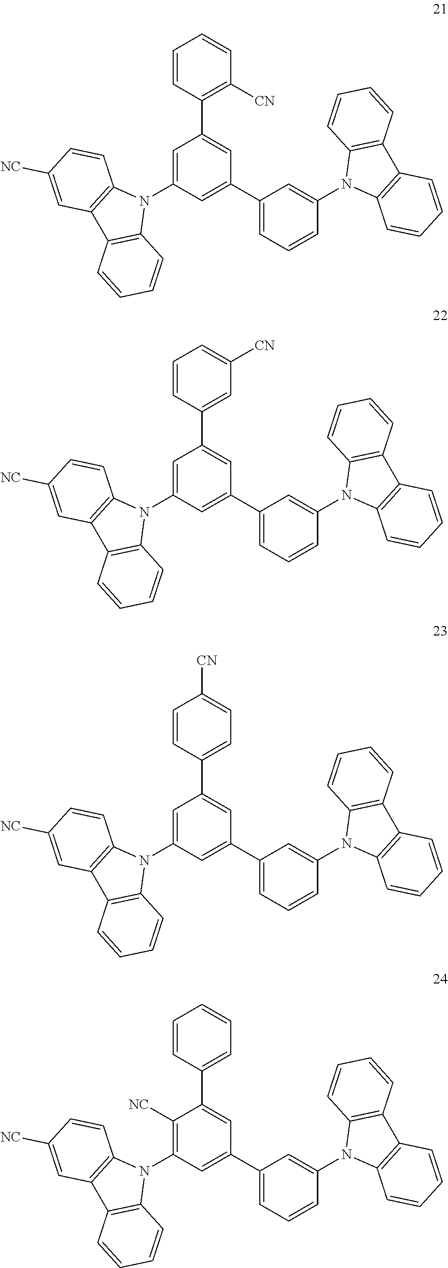 [SMILES: N#Cc1ccc2c(c1)c1ccccc1n2-c1cc(-c2cccc(-n3c4ccccc4c4ccccc43)c2)cc(-c2ccccc2C#N)c1 ; N#Cc1cccc(-c2cc(-c3cccc(-n4c5ccccc5c5ccccc54)c3)cc(-n3c4ccccc4c4cc(C#N)ccc43)c2)c1 ; N#Cc1ccc(-c2cc(-c3cccc(-n4c5ccccc5c5ccccc54)c3)cc(-n3c4ccccc4c4cc(C#N)ccc43)c2)cc1 ; N#Cc1ccc2c(c1)c1ccccc1n2-c1cc(-c2cccc(-n3c4ccccc4c4ccccc43)c2)cc(-c2ccccc2)c1C#N]

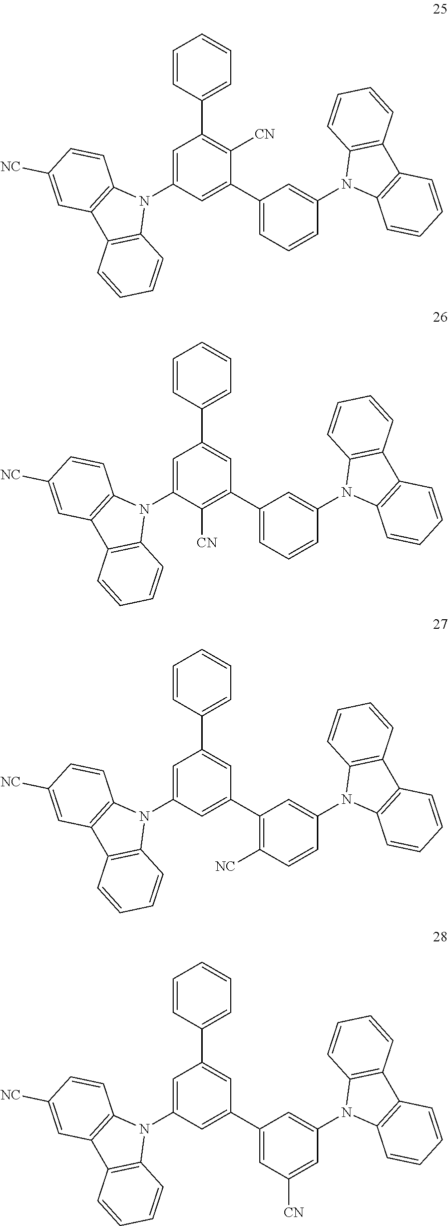 [SMILES: N#Cc1ccc2c(c1)c1ccccc1n2-c1cc(-c2ccccc2)c(C#N)c(-c2cccc(-n3c4ccccc4c4ccccc43)c2)c1 ; N#Cc1ccc2c(c1)c1ccccc1n2-c1cc(-c2ccccc2)cc(-c2cccc(-n3c4ccccc4c4ccccc43)c2)c1C#N ; N#Cc1ccc2c(c1)c1ccccc1n2-c1cc(-c2ccccc2)cc(-c2cc(-n3c4ccccc4c4ccccc43)ccc2C#N)c1 ; N#Cc1cc(-c2cc(-c3ccccc3)cc(-n3c4ccccc4c4cc(C#N)ccc43)c2)cc(-n2c3ccccc3c3ccccc32)c1]

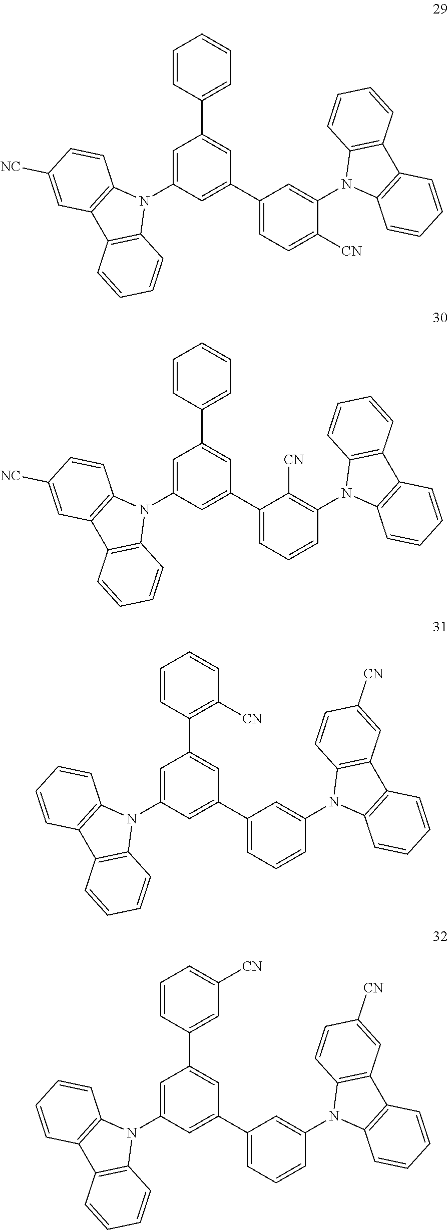 [SMILES: N#Cc1ccc2c(c1)c1ccccc1n2-c1cc(-c2ccccc2)cc(-c2ccc(C#N)c(-n3c4ccccc4c4ccccc43)c2)c1 ; N#Cc1ccc2c(c1)c1ccccc1n2-c1cc(-c2ccccc2)cc(-c2cccc(-n3c4ccccc4c4ccccc43)c2C#N)c1 ; N#Cc1ccc2c(c1)c1ccccc1n2-c1cccc(-c2cc(-c3ccccc3C#N)cc(-n3c4ccccc4c4ccccc43)c2)c1 ; N#Cc1cccc(-c2cc(-c3cccc(-n4c5ccccc5c5cc(C#N)ccc54)c3)cc(-n3c4ccccc4c4ccccc43)c2)c1]

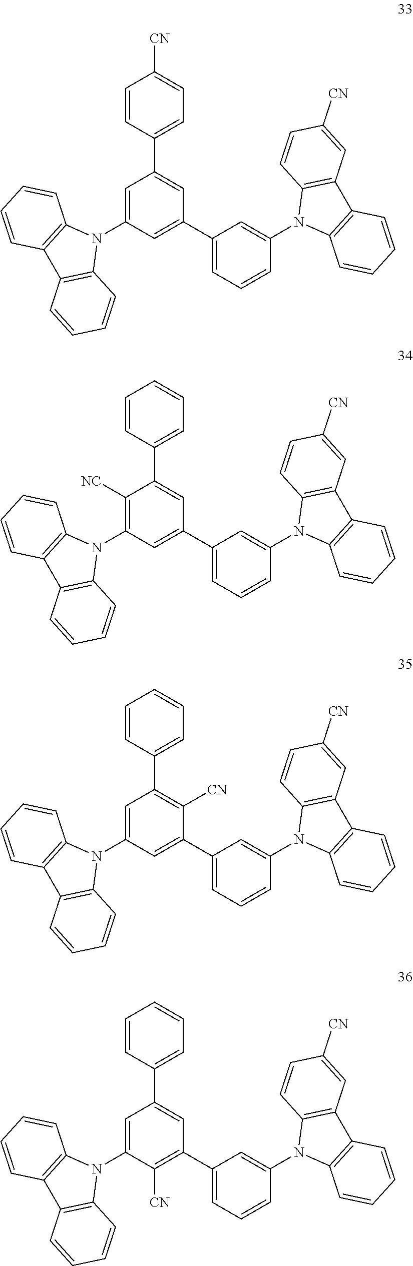 [SMILES: N#Cc1ccc(-c2cc(-c3cccc(-n4c5ccccc5c5cc(C#N)ccc54)c3)cc(-n3c4ccccc4c4ccccc43)c2)cc1 ; N#Cc1ccc2c(c1)c1ccccc1n2-c1cccc(-c2cc(-c3ccccc3)c(C#N)c(-n3c4ccccc4c4ccccc43)c2)c1 ; N#Cc1ccc2c(c1)c1ccccc1n2-c1cccc(-c2cc(-n3c4ccccc4c4ccccc43)cc(-c3ccccc3)c2C#N)c1 ; N#Cc1ccc2c(c1)c1ccccc1n2-c1cccc(-c2cc(-c3ccccc3)cc(-n3c4ccccc4c4ccccc43)c2C#N)c1]

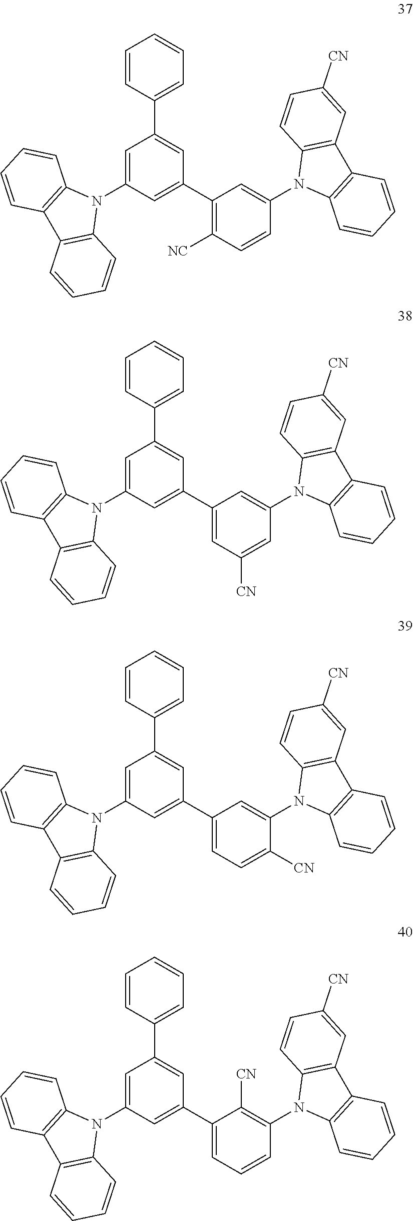 [SMILES: N#Cc1ccc2c(c1)c1ccccc1n2-c1ccc(C#N)c(-c2cc(-c3ccccc3)cc(-n3c4ccccc4c4ccccc43)c2)c1 ; N#Cc1cc(-c2cc(-c3ccccc3)cc(-n3c4ccccc4c4ccccc43)c2)cc(-n2c3ccccc3c3cc(C#N)ccc32)c1 ; N#Cc1ccc2c(c1)c1ccccc1n2-c1cc(-c2cc(-c3ccccc3)cc(-n3c4ccccc4c4ccccc43)c2)ccc1C#N ; N#Cc1ccc2c(c1)c1ccccc1n2-c1cccc(-c2cc(-c3ccccc3)cc(-n3c4ccccc4c4ccccc43)c2)c1C#N]

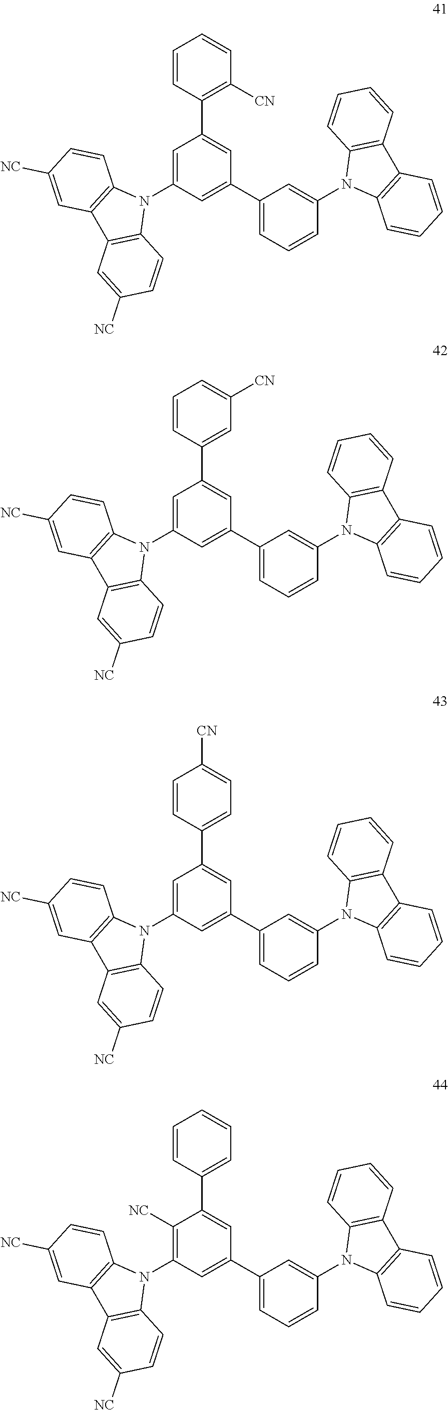 [SMILES: N#Cc1ccc2c(c1)c1cc(C#N)ccc1n2-c1cc(-c2cccc(-n3c4ccccc4c4ccccc43)c2)cc(-c2ccccc2C#N)c1 ; N#Cc1cccc(-c2cc(-c3cccc(-n4c5ccccc5c5ccccc54)c3)cc(-n3c4ccc(C#N)cc4c4cc(C#N)ccc43)c2)c1 ; N#Cc1ccc(-c2cc(-c3cccc(-n4c5ccccc5c5ccccc54)c3)cc(-n3c4ccc(C#N)cc4c4cc(C#N)ccc43)c2)cc1 ; N#Cc1ccc2c(c1)c1cc(C#N)ccc1n2-c1cc(-c2cccc(-n3c4ccccc4c4ccccc43)c2)cc(-c2ccccc2)c1C#N]

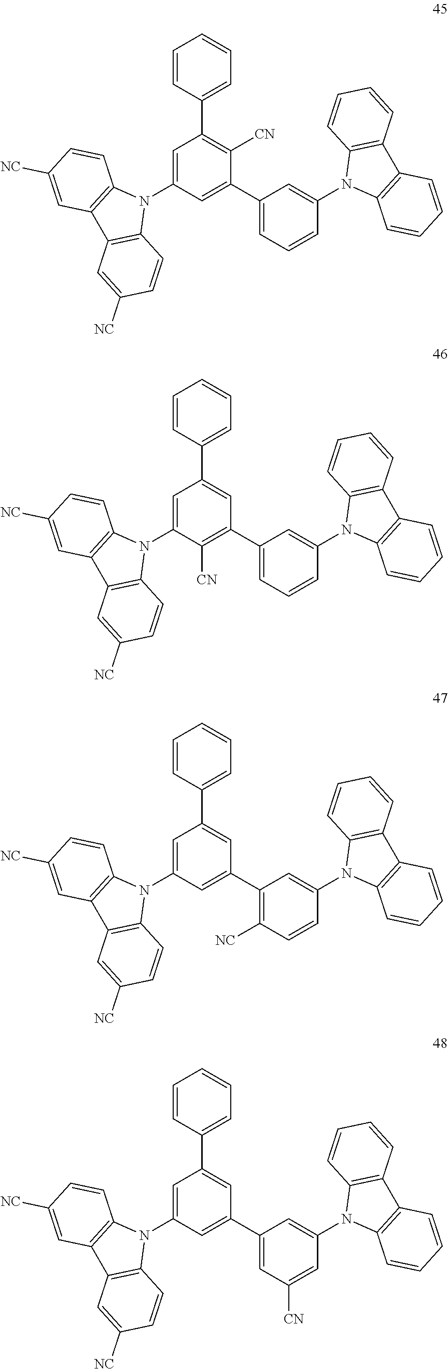 [SMILES: N#Cc1ccc2c(c1)c1cc(C#N)ccc1n2-c1cc(-c2ccccc2)c(C#N)c(-c2cccc(-n3c4ccccc4c4ccccc43)c2)c1 ; N#Cc1ccc2c(c1)c1cc(C#N)ccc1n2-c1cc(-c2ccccc2)cc(-c2cccc(-n3c4ccccc4c4ccccc43)c2)c1C#N ; N#Cc1ccc2c(c1)c1cc(C#N)ccc1n2-c1cc(-c2ccccc2)cc(-c2cc(-n3c4ccccc4c4ccccc43)ccc2C#N)c1 ; N#Cc1cc(-c2cc(-c3ccccc3)cc(-n3c4ccc(C#N)cc4c4cc(C#N)ccc43)c2)cc(-n2c3ccccc3c3ccccc32)c1]

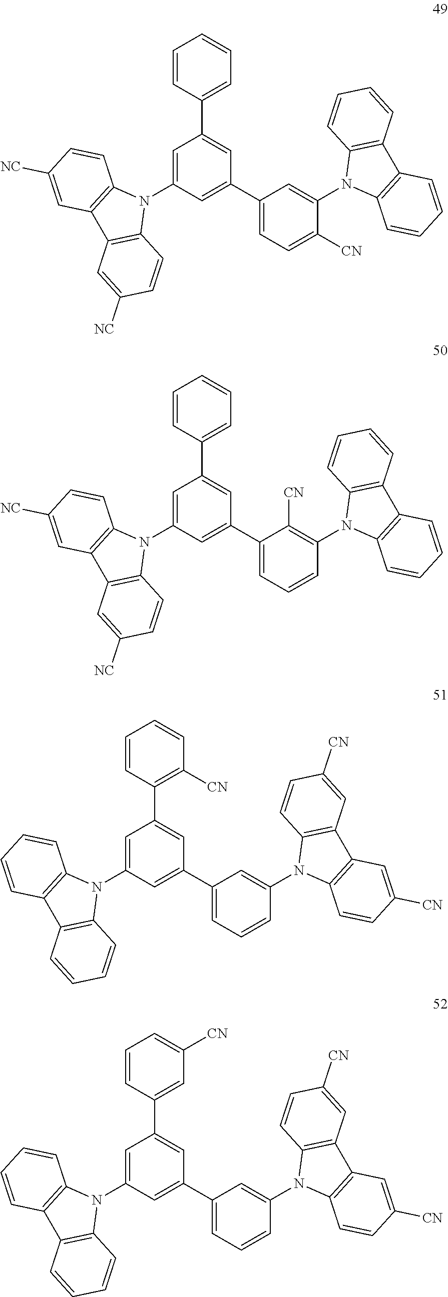 [SMILES: N#Cc1ccc2c(c1)c1cc(C#N)ccc1n2-c1cc(-c2ccccc2)cc(-c2ccc(C#N)c(-n3c4ccccc4c4ccccc43)c2)c1 ; N#Cc1ccc2c(c1)c1cc(C#N)ccc1n2-c1cc(-c2ccccc2)cc(-c2cccc(-n3c4ccccc4c4ccccc43)c2C#N)c1 ; N#Cc1ccc2c(c1)c1cc(C#N)ccc1n2-c1cccc(-c2cc(-c3ccccc3C#N)cc(-n3c4ccccc4c4ccccc43)c2)c1 ; N#Cc1cccc(-c2cc(-c3cccc(-n4c5ccc(C#N)cc5c5cc(C#N)ccc54)c3)cc(-n3c4ccccc4c4ccccc43)c2)c1]

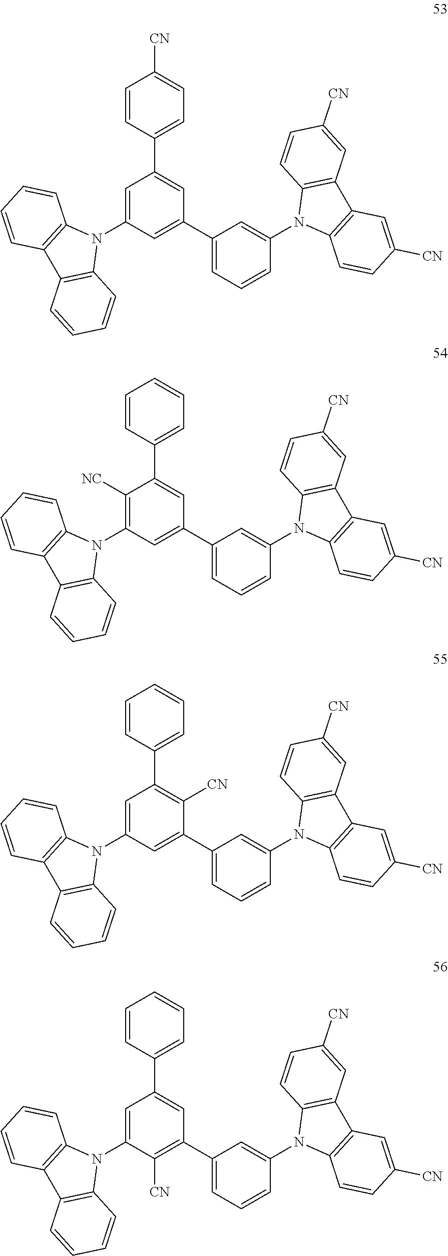 [SMILES: N#Cc1ccc(-c2cc(-c3cccc(-n4c5ccc(C#N)cc5c5cc(C#N)ccc54)c3)cc(-n3c4ccccc4c4ccccc43)c2)cc1 ; N#Cc1ccc2c(c1)c1cc(C#N)ccc1n2-c1cccc(-c2cc(-c3ccccc3)c(C#N)c(-n3c4ccccc4c4ccccc43)c2)c1 ; N#Cc1ccc2c(c1)c1cc(C#N)ccc1n2-c1cccc(-c2cc(-n3c4ccccc4c4ccccc43)cc(-c3ccccc3)c2C#N)c1 ; N#Cc1ccc2c(c1)c1cc(C#N)ccc1n2-c1cccc(-c2cc(-c3ccccc3)cc(-n3c4ccccc4c4ccccc43)c2C#N)c1]

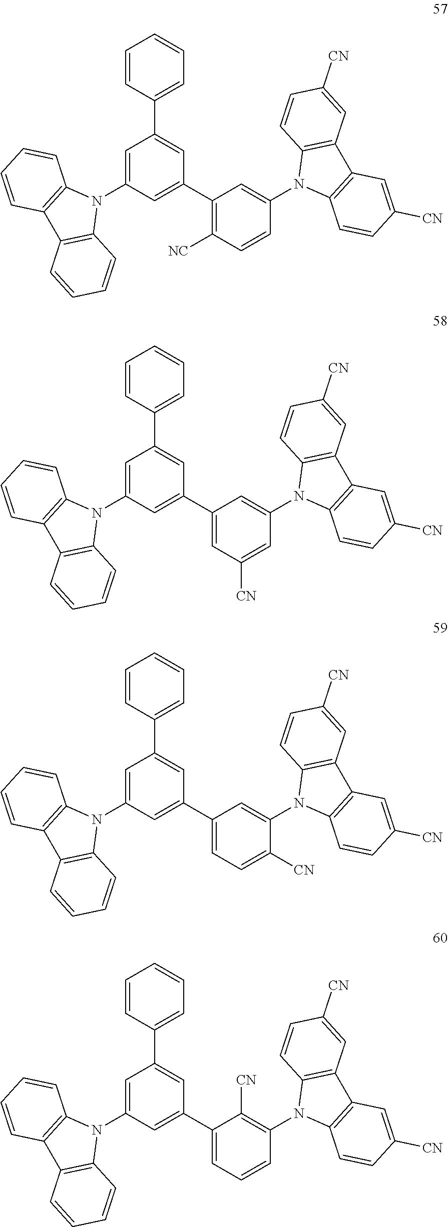 [SMILES: N#Cc1ccc2c(c1)c1cc(C#N)ccc1n2-c1ccc(C#N)c(-c2cc(-c3ccccc3)cc(-n3c4ccccc4c4ccccc43)c2)c1 ; N#Cc1cc(-c2cc(-c3ccccc3)cc(-n3c4ccccc4c4ccccc43)c2)cc(-n2c3ccc(C#N)cc3c3cc(C#N)ccc32)c1 ; N#Cc1ccc2c(c1)c1cc(C#N)ccc1n2-c1cc(-c2cc(-c3ccccc3)cc(-n3c4ccccc4c4ccccc43)c2)ccc1C#N ; N#Cc1ccc2c(c1)c1cc(C#N)ccc1n2-c1cccc(-c2cc(-c3ccccc3)cc(-n3c4ccccc4c4ccccc43)c2)c1C#N]

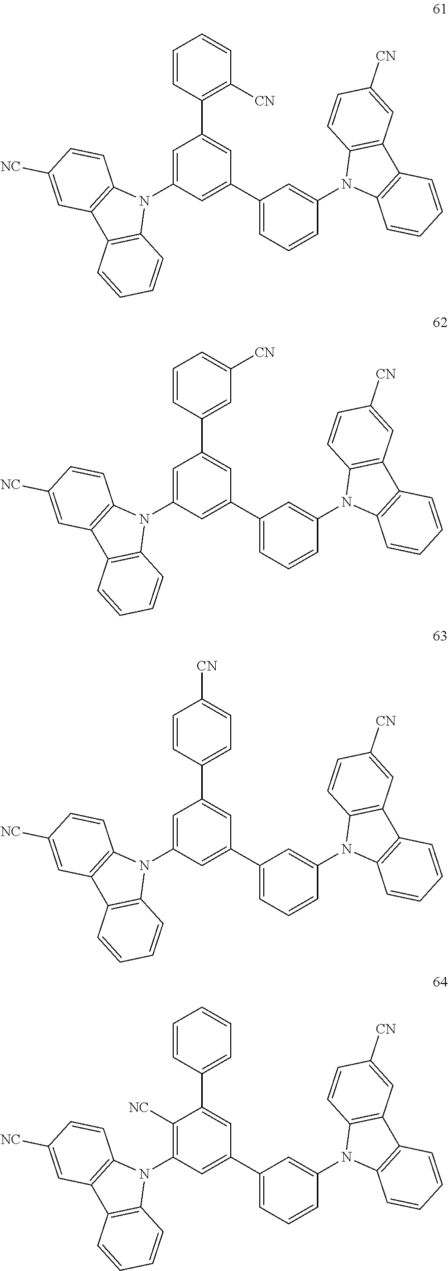 [SMILES: N#Cc1ccc2c(c1)c1ccccc1n2-c1cccc(-c2cc(-c3ccccc3C#N)cc(-n3c4ccccc4c4cc(C#N)ccc43)c2)c1 ; N#Cc1cccc(-c2cc(-c3cccc(-n4c5ccccc5c5cc(C#N)ccc54)c3)cc(-n3c4ccccc4c4cc(C#N)ccc43)c2)c1 ; N#Cc1ccc(-c2cc(-c3cccc(-n4c5ccccc5c5cc(C#N)ccc54)c3)cc(-n3c4ccccc4c4cc(C#N)ccc43)c2)cc1 ; N#Cc1ccc2c(c1)c1ccccc1n2-c1cccc(-c2cc(-c3ccccc3)c(C#N)c(-n3c4ccccc4c4cc(C#N)ccc43)c2)c1]

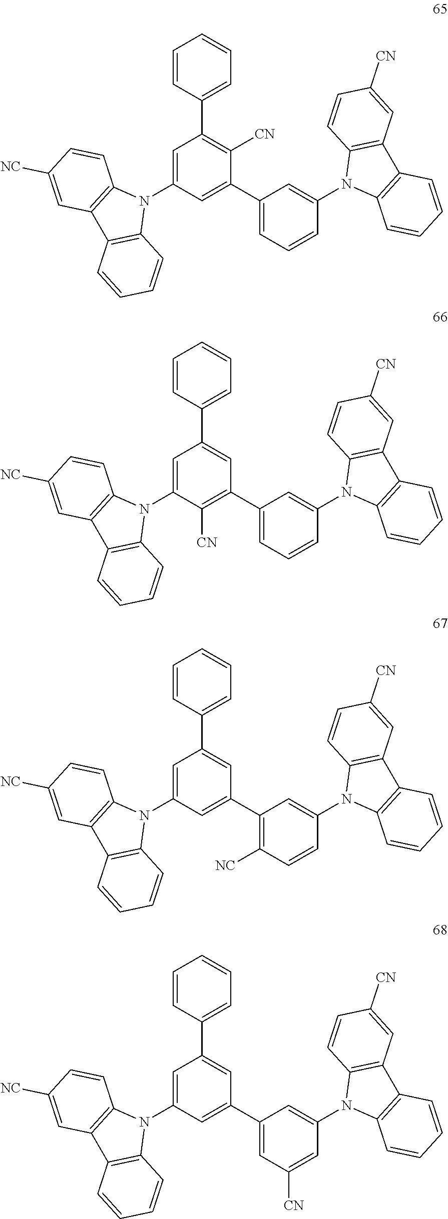 [SMILES: N#Cc1ccc2c(c1)c1ccccc1n2-c1cccc(-c2cc(-n3c4ccccc4c4cc(C#N)ccc43)cc(-c3ccccc3)c2C#N)c1 ; N#Cc1ccc2c(c1)c1ccccc1n2-c1cccc(-c2cc(-c3ccccc3)cc(-n3c4ccccc4c4cc(C#N)ccc43)c2C#N)c1 ; N#Cc1ccc2c(c1)c1ccccc1n2-c1cc(-c2ccccc2)cc(-c2cc(-n3c4ccccc4c4cc(C#N)ccc43)ccc2C#N)c1 ; N#Cc1cc(-c2cc(-c3ccccc3)cc(-n3c4ccccc4c4cc(C#N)ccc43)c2)cc(-n2c3ccccc3c3cc(C#N)ccc32)c1]

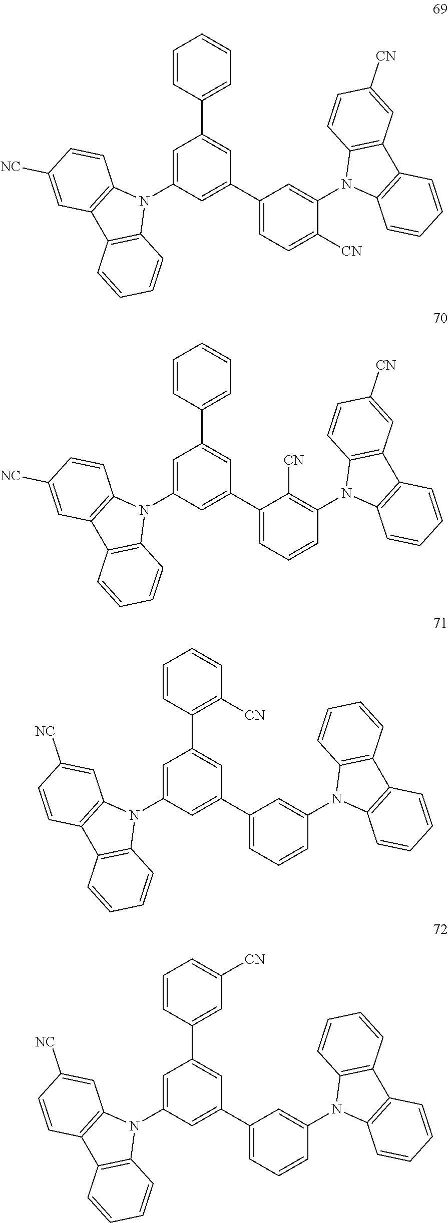 [SMILES: N#Cc1ccc2c(c1)c1ccccc1n2-c1cc(-c2ccccc2)cc(-c2ccc(C#N)c(-n3c4ccccc4c4cc(C#N)ccc43)c2)c1 ; N#Cc1ccc2c(c1)c1ccccc1n2-c1cc(-c2ccccc2)cc(-c2cccc(-n3c4ccccc4c4cc(C#N)ccc43)c2C#N)c1 ; N#Cc1ccc2c3ccccc3n(-c3cc(-c4cccc(-n5c6ccccc6c6ccccc65)c4)cc(-c4ccccc4C#N)c3)c2c1 ; N#Cc1cccc(-c2cc(-c3cccc(-n4c5ccccc5c5ccccc54)c3)cc(-n3c4ccccc4c4ccc(C#N)cc43)c2)c1]

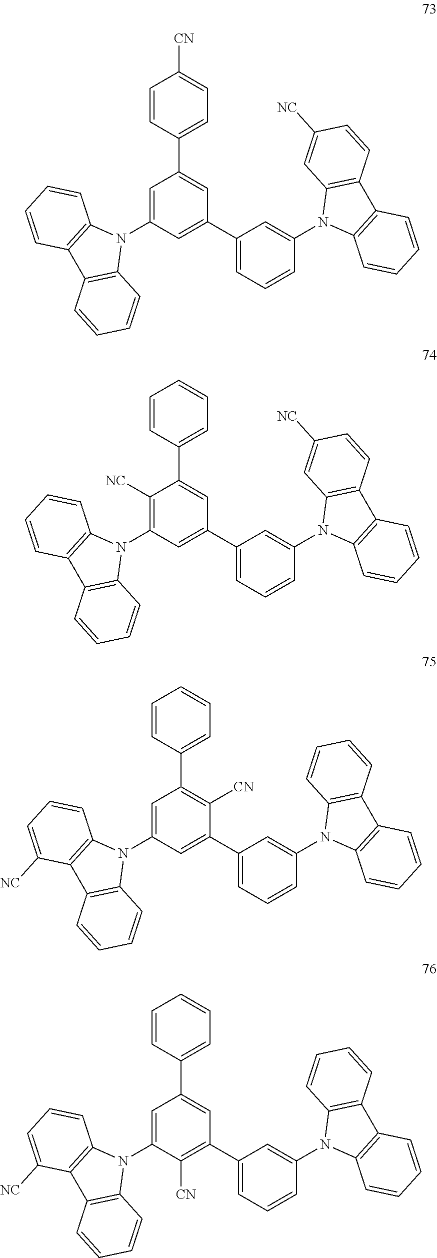 [SMILES: N#Cc1ccc(-c2cc(-c3cccc(-n4c5ccccc5c5ccc(C#N)cc54)c3)cc(-n3c4ccccc4c4ccccc43)c2)cc1 ; N#Cc1ccc2c3ccccc3n(-c3cccc(-c4cc(-c5ccccc5)c(C#N)c(-n5c6ccccc6c6ccccc65)c4)c3)c2c1 ; N#Cc1c(-c2ccccc2)cc(-n2c3ccccc3c3c(C#N)cccc32)cc1-c1cccc(-n2c3ccccc3c3ccccc32)c1 ; N#Cc1c(-c2cccc(-n3c4ccccc4c4ccccc43)c2)cc(-c2ccccc2)cc1-n1c2ccccc2c2c(C#N)cccc21]

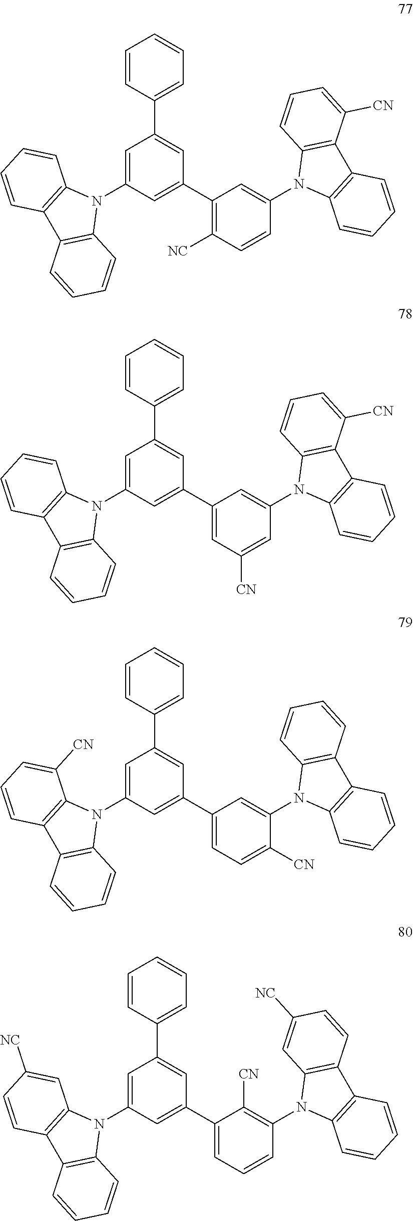 [SMILES: N#Cc1ccc(-n2c3ccccc3c3c(C#N)cccc32)cc1-c1cc(-c2ccccc2)cc(-n2c3ccccc3c3ccccc32)c1 ; N#Cc1cc(-c2cc(-c3ccccc3)cc(-n3c4ccccc4c4ccccc43)c2)cc(-n2c3ccccc3c3c(C#N)cccc32)c1 ; N#Cc1ccc(-c2cc(-c3ccccc3)cc(-n3c4ccccc4c4cccc(C#N)c43)c2)cc1-n1c2ccccc2c2ccccc21 ; N#Cc1ccc2c3ccccc3n(-c3cc(-c4ccccc4)cc(-c4cccc(-n5c6ccccc6c6ccc(C#N)cc65)c4C#N)c3)c2c1]

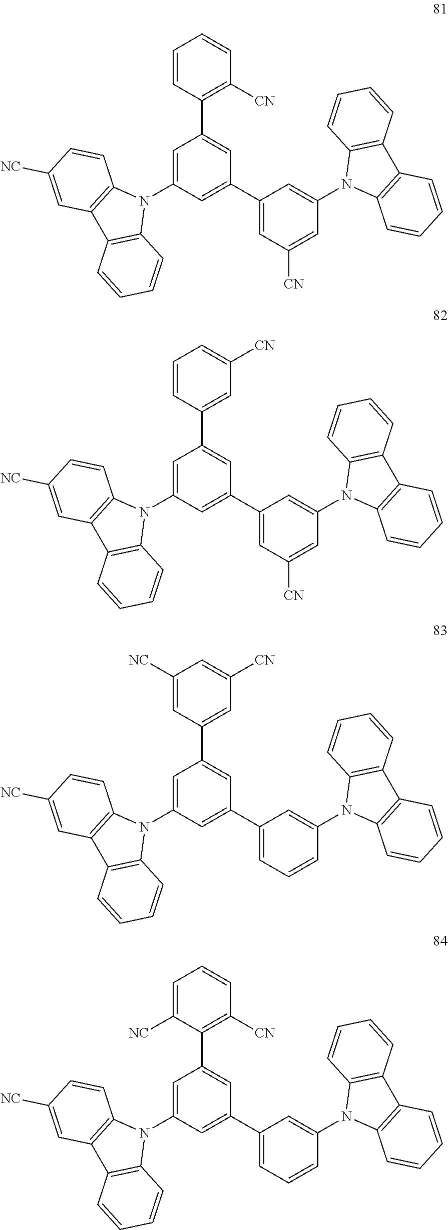 [SMILES: N#Cc1cc(-c2cc(-c3ccccc3C#N)cc(-n3c4ccccc4c4cc(C#N)ccc43)c2)cc(-n2c3ccccc3c3ccccc32)c1 ; N#Cc1cccc(-c2cc(-c3cc(C#N)cc(-n4c5ccccc5c5ccccc54)c3)cc(-n3c4ccccc4c4cc(C#N)ccc43)c2)c1 ; N#Cc1cc(C#N)cc(-c2cc(-c3cccc(-n4c5ccccc5c5ccccc54)c3)cc(-n3c4ccccc4c4cc(C#N)ccc43)c2)c1 ; N#Cc1ccc2c(c1)c1ccccc1n2-c1cc(-c2cccc(-n3c4ccccc4c4ccccc43)c2)cc(-c2c(C#N)cccc2C#N)c1]

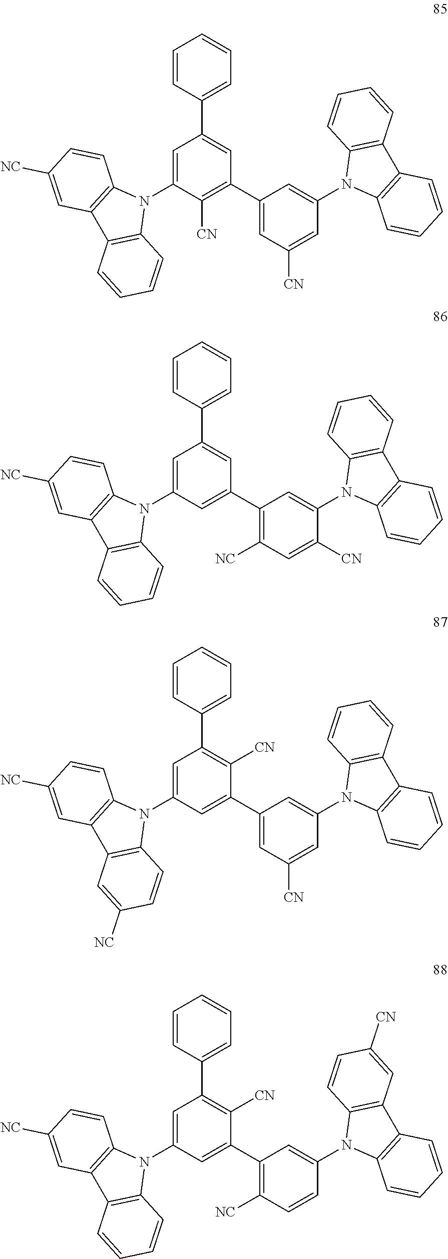 [SMILES: N#Cc1cc(-c2cc(-c3ccccc3)cc(-n3c4ccccc4c4cc(C#N)ccc43)c2C#N)cc(-n2c3ccccc3c3ccccc32)c1 ; N#Cc1ccc2c(c1)c1ccccc1n2-c1cc(-c2ccccc2)cc(-c2cc(-n3c4ccccc4c4ccccc43)c(C#N)cc2C#N)c1 ; N#Cc1cc(-c2cc(-n3c4ccc(C#N)cc4c4cc(C#N)ccc43)cc(-c3ccccc3)c2C#N)cc(-n2c3ccccc3c3ccccc32)c1 ; N#Cc1ccc2c(c1)c1ccccc1n2-c1ccc(C#N)c(-c2cc(-n3c4ccccc4c4cc(C#N)ccc43)cc(-c3ccccc3)c2C#N)c1]

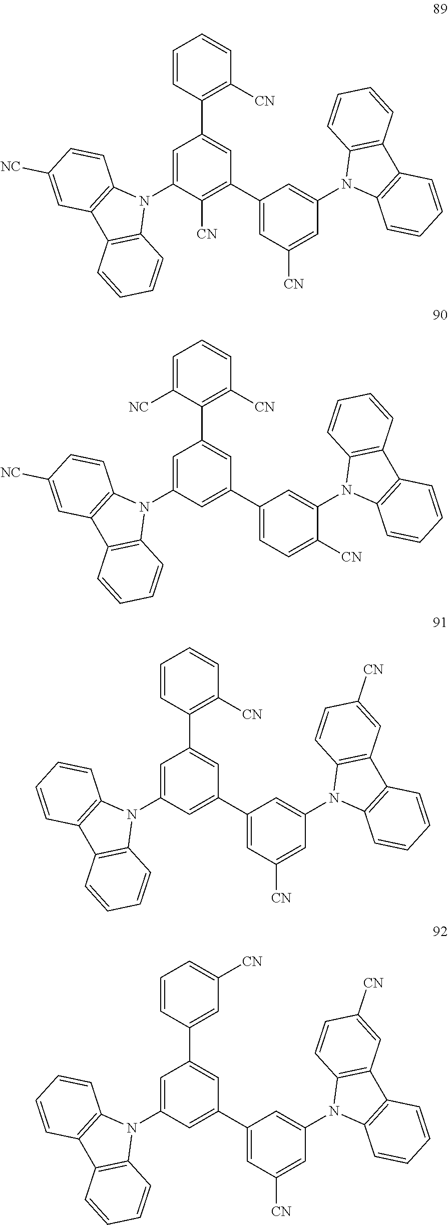 [SMILES: N#Cc1cc(-c2cc(-c3ccccc3C#N)cc(-n3c4ccccc4c4cc(C#N)ccc43)c2C#N)cc(-n2c3ccccc3c3ccccc32)c1 ; N#Cc1ccc2c(c1)c1ccccc1n2-c1cc(-c2ccc(C#N)c(-n3c4ccccc4c4ccccc43)c2)cc(-c2c(C#N)cccc2C#N)c1 ; N#Cc1cc(-c2cc(-c3ccccc3C#N)cc(-n3c4ccccc4c4ccccc43)c2)cc(-n2c3ccccc3c3cc(C#N)ccc32)c1 ; N#Cc1cccc(-c2cc(-c3cc(C#N)cc(-n4c5ccccc5c5cc(C#N)ccc54)c3)cc(-n3c4ccccc4c4ccccc43)c2)c1]

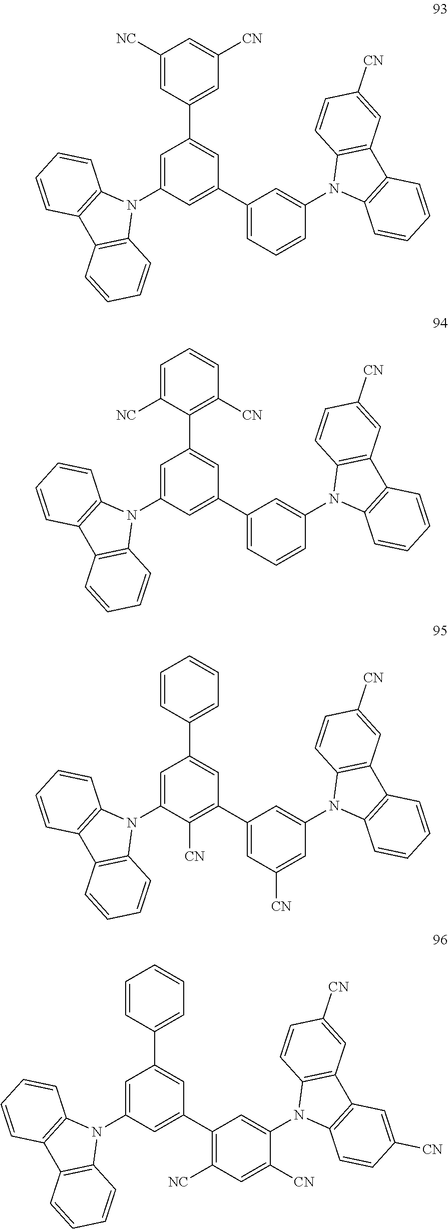 [SMILES: N#Cc1cc(C#N)cc(-c2cc(-c3cccc(-n4c5ccccc5c5cc(C#N)ccc54)c3)cc(-n3c4ccccc4c4ccccc43)c2)c1 ; N#Cc1ccc2c(c1)c1ccccc1n2-c1cccc(-c2cc(-c3c(C#N)cccc3C#N)cc(-n3c4ccccc4c4ccccc43)c2)c1 ; N#Cc1cc(-c2cc(-c3ccccc3)cc(-n3c4ccccc4c4ccccc43)c2C#N)cc(-n2c3ccccc3c3cc(C#N)ccc32)c1 ; N#Cc1ccc2c(c1)c1cc(C#N)ccc1n2-c1cc(-c2cc(-c3ccccc3)cc(-n3c4ccccc4c4ccccc43)c2)c(C#N)cc1C#N]

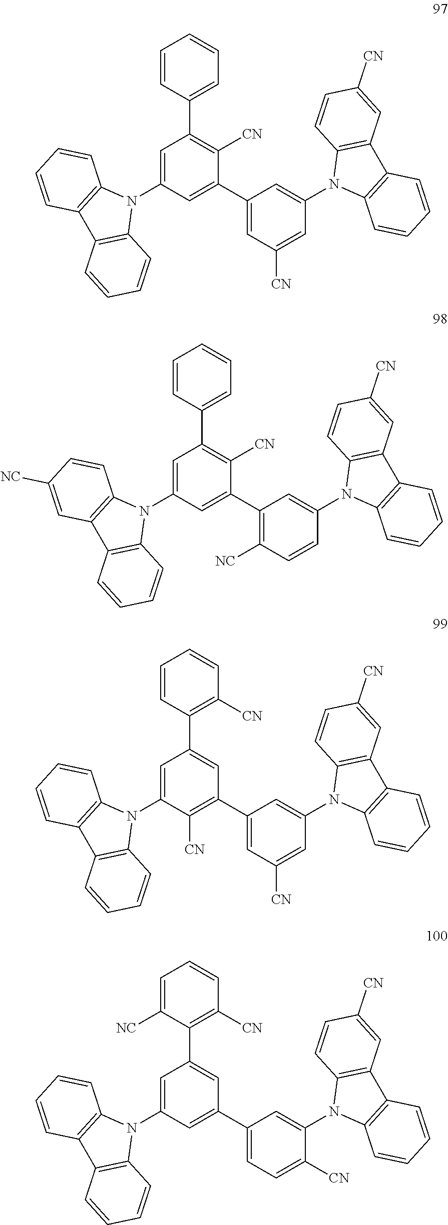 [SMILES: N#Cc1cc(-c2cc(-n3c4ccccc4c4ccccc43)cc(-c3ccccc3)c2C#N)cc(-n2c3ccccc3c3cc(C#N)ccc32)c1 ; N#Cc1ccc2c(c1)c1ccccc1n2-c1ccc(C#N)c(-c2cc(-n3c4ccccc4c4cc(C#N)ccc43)cc(-c3ccccc3)c2C#N)c1 ; N#Cc1cc(-c2cc(-c3ccccc3C#N)cc(-n3c4ccccc4c4ccccc43)c2C#N)cc(-n2c3ccccc3c3cc(C#N)ccc32)c1 ; N#Cc1ccc2c(c1)c1ccccc1n2-c1cc(-c2cc(-c3c(C#N)cccc3C#N)cc(-n3c4ccccc4c4ccccc43)c2)ccc1C#N]

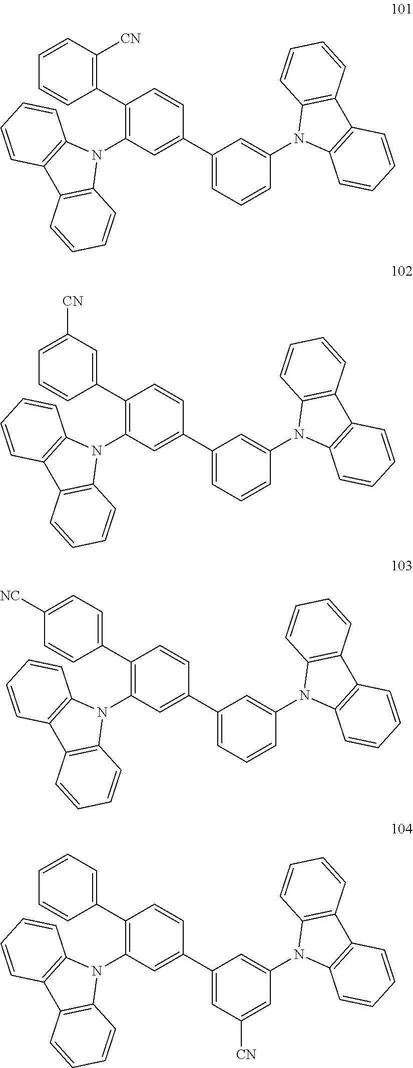

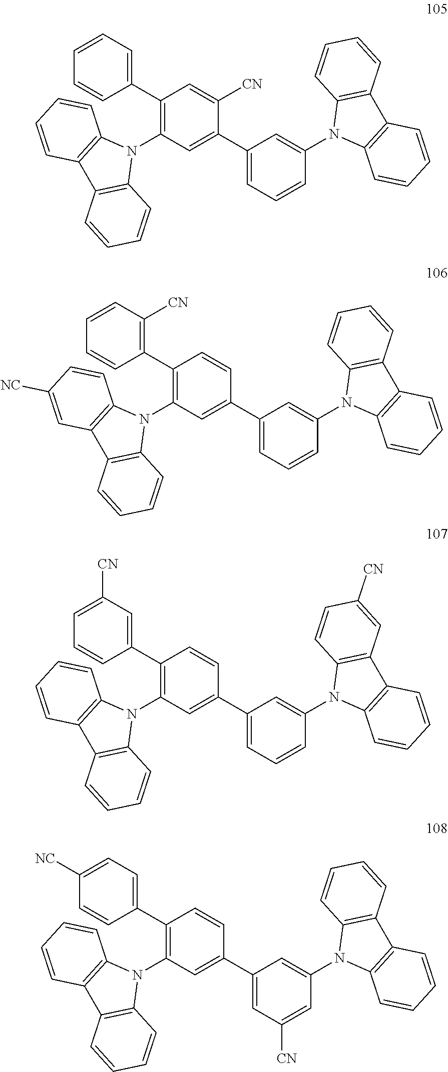 [SMILES: N#Cc1cc(-c2ccccc2)c(-n2c3ccccc3c3ccccc32)cc1-c1cccc(-n2c3ccccc3c3ccccc32)c1 ; N#Cc1ccc2c(c1)c1ccccc1n2-c1cc(-c2cccc(-n3c4ccccc4c4ccccc43)c2)ccc1-c1ccccc1C#N ; N#Cc1cccc(-c2ccc(-c3cccc(-n4c5ccccc5c5cc(C#N)ccc54)c3)cc2-n2c3ccccc3c3ccccc32)c1 ; N#Cc1ccc(-c2ccc(-c3cc(C#N)cc(-n4c5ccccc5c5ccccc54)c3)cc2-n2c3ccccc3c3ccccc32)cc1]

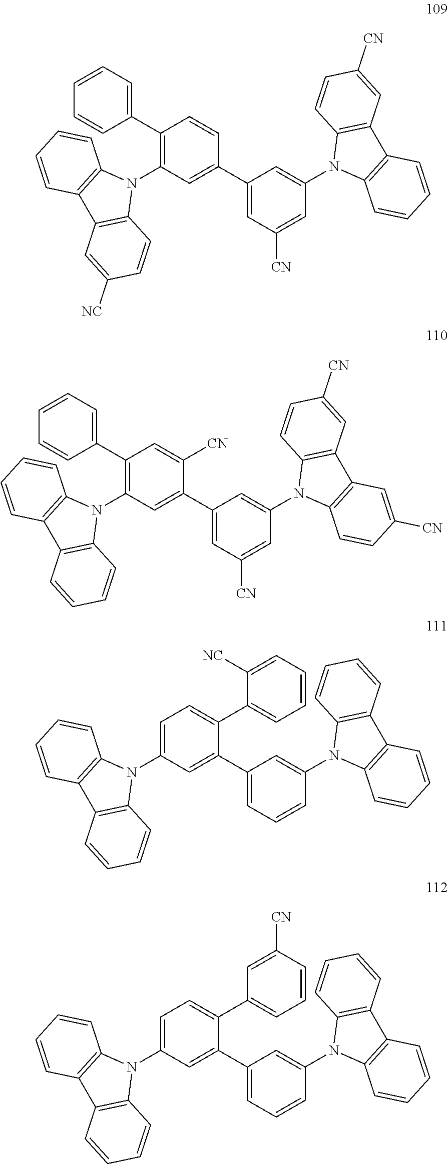 [SMILES: N#Cc1cc(-c2ccc(-c3ccccc3)c(-n3c4ccccc4c4cc(C#N)ccc43)c2)cc(-n2c3ccccc3c3cc(C#N)ccc32)c1 ; N#Cc1cc(-c2cc(-n3c4ccccc4c4ccccc43)c(-c3ccccc3)cc2C#N)cc(-n2c3ccc(C#N)cc3c3cc(C#N)ccc32)c1 ; N#Cc1ccccc1-c1ccc(-n2c3ccccc3c3ccccc32)cc1-c1cccc(-n2c3ccccc3c3ccccc32)c1 ; N#Cc1cccc(-c2ccc(-n3c4ccccc4c4ccccc43)cc2-c2cccc(-n3c4ccccc4c4ccccc43)c2)c1]

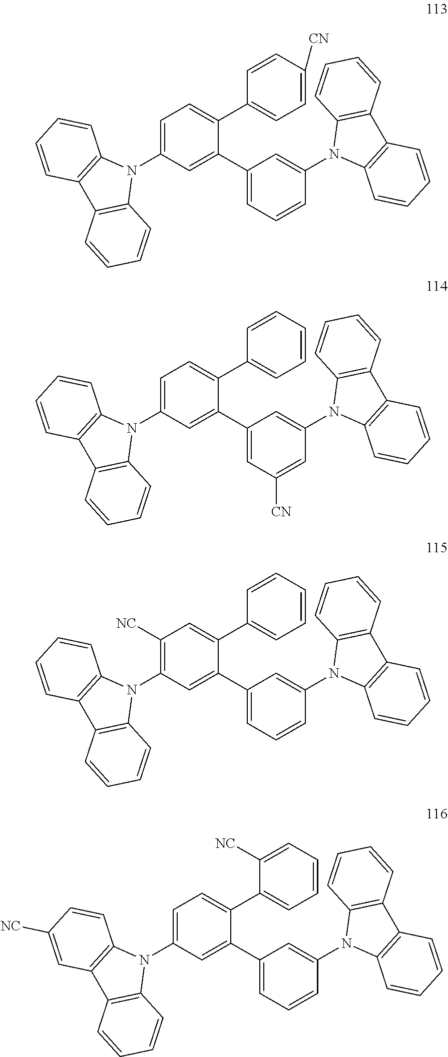 [SMILES: N#Cc1ccc(-c2ccc(-n3c4ccccc4c4ccccc43)cc2-c2cccc(-n3c4ccccc4c4ccccc43)c2)cc1 ; N#Cc1cc(-c2cc(-n3c4ccccc4c4ccccc43)ccc2-c2ccccc2)cc(-n2c3ccccc3c3ccccc32)c1 ; N#Cc1cc(-c2ccccc2)c(-c2cccc(-n3c4ccccc4c4ccccc43)c2)cc1-n1c2ccccc2c2ccccc21 ; N#Cc1ccc2c(c1)c1ccccc1n2-c1ccc(-c2ccccc2C#N)c(-c2cccc(-n3c4ccccc4c4ccccc43)c2)c1]

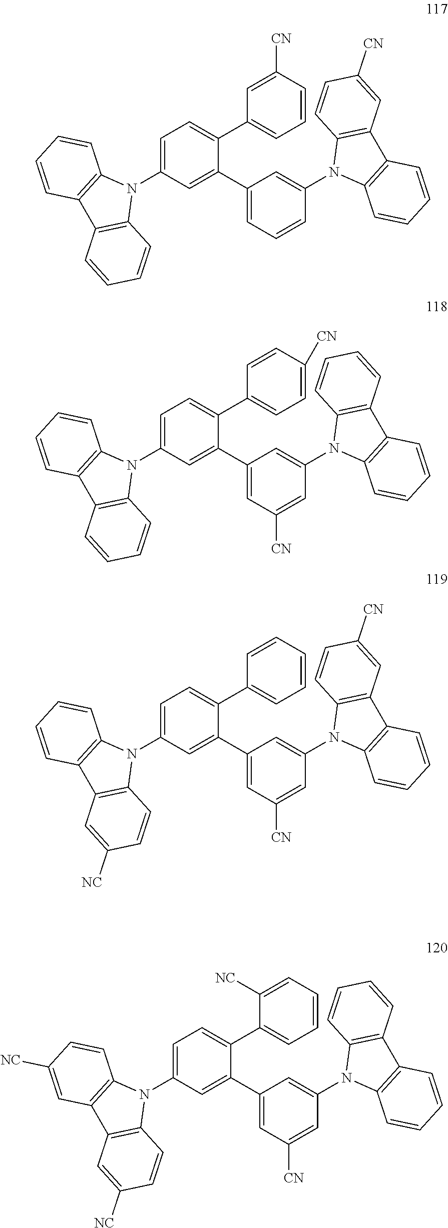 [SMILES: N#Cc1cccc(-c2ccc(-n3c4ccccc4c4ccccc43)cc2-c2cccc(-n3c4ccccc4c4cc(C#N)ccc43)c2)c1 ; N#Cc1ccc(-c2ccc(-n3c4ccccc4c4ccccc43)cc2-c2cc(C#N)cc(-n3c4ccccc4c4ccccc43)c2)cc1 ; N#Cc1cc(-c2cc(-n3c4ccccc4c4cc(C#N)ccc43)ccc2-c2ccccc2)cc(-n2c3ccccc3c3cc(C#N)ccc32)c1 ; N#Cc1cc(-c2cc(-n3c4ccc(C#N)cc4c4cc(C#N)ccc43)ccc2-c2ccccc2C#N)cc(-n2c3ccccc3c3ccccc32)c1]

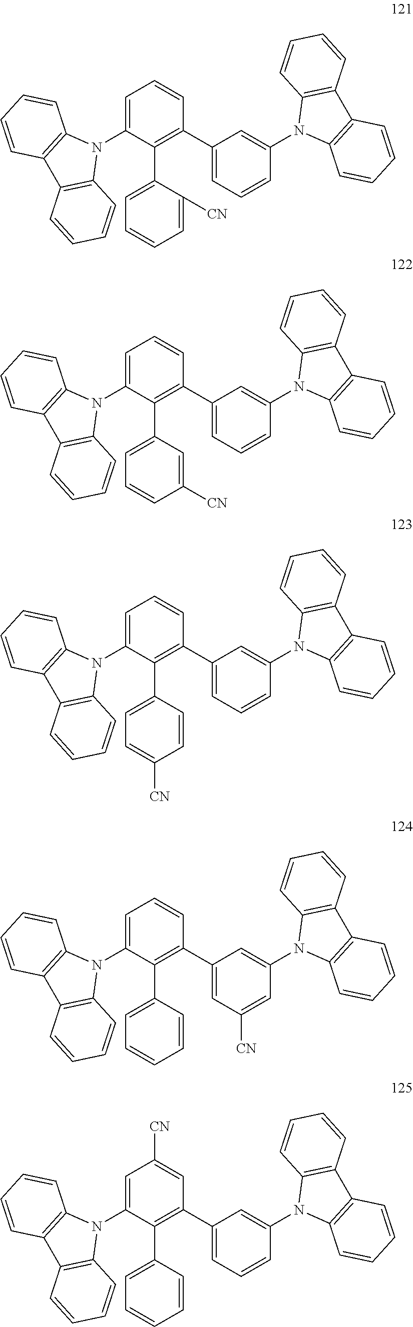 [SMILES: N#Cc1ccccc1-c1c(-c2cccc(-n3c4ccccc4c4ccccc43)c2)cccc1-n1c2ccccc2c2ccccc21 ; N#Cc1cccc(-c2c(-c3cccc(-n4c5ccccc5c5ccccc54)c3)cccc2-n2c3ccccc3c3ccccc32)c1 ; N#Cc1ccc(-c2c(-c3cccc(-n4c5ccccc5c5ccccc54)c3)cccc2-n2c3ccccc3c3ccccc32)cc1 ; N#Cc1cc(-c2cccc(-n3c4ccccc4c4ccccc43)c2-c2ccccc2)cc(-n2c3ccccc3c3ccccc32)c1 ; N#Cc1cc(-c2cccc(-n3c4ccccc4c4ccccc43)c2)c(-c2ccccc2)c(-n2c3ccccc3c3ccccc32)c1]

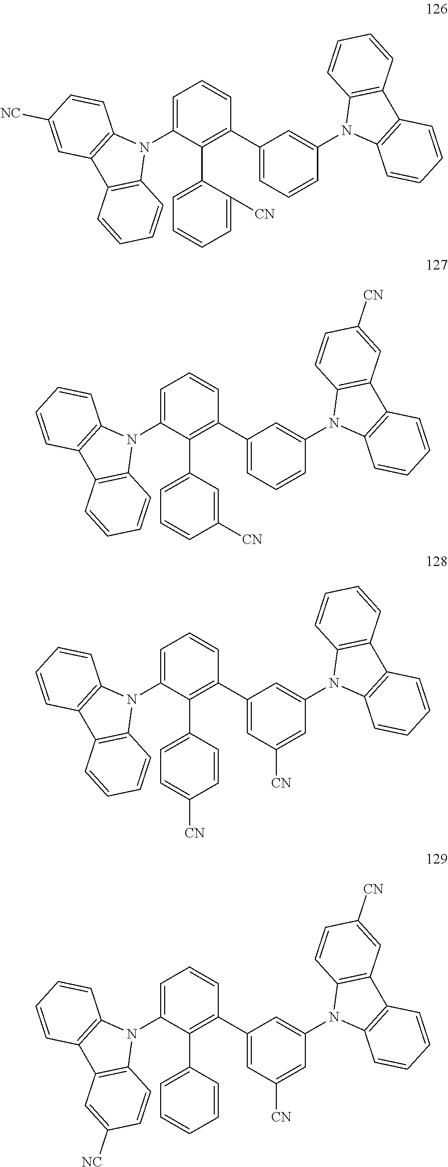 [SMILES: N#Cc1ccc2c(c1)c1ccccc1n2-c1cccc(-c2cccc(-n3c4ccccc4c4ccccc43)c2)c1-c1ccccc1C#N ; N#Cc1cccc(-c2c(-c3cccc(-n4c5ccccc5c5cc(C#N)ccc54)c3)cccc2-n2c3ccccc3c3ccccc32)c1 ; N#Cc1ccc(-c2c(-c3cc(C#N)cc(-n4c5ccccc5c5ccccc54)c3)cccc2-n2c3ccccc3c3ccccc32)cc1 ; N#Cc1cc(-c2cccc(-n3c4ccccc4c4cc(C#N)ccc43)c2-c2ccccc2)cc(-n2c3ccccc3c3cc(C#N)ccc32)c1]

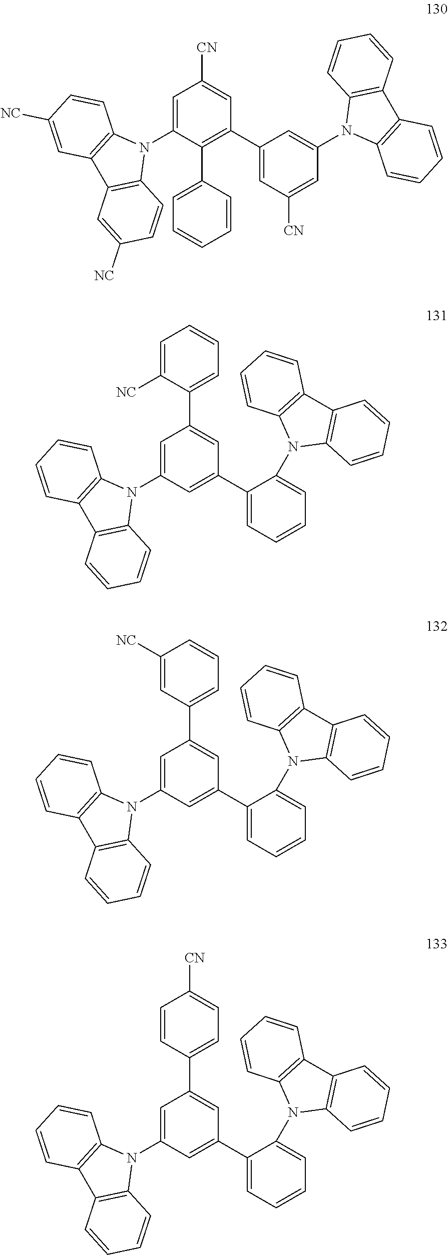 [SMILES: N#Cc1cc(-c2cc(C#N)cc(-n3c4ccc(C#N)cc4c4cc(C#N)ccc43)c2-c2ccccc2)cc(-n2c3ccccc3c3ccccc32)c1 ; N#Cc1ccccc1-c1cc(-c2ccccc2-n2c3ccccc3c3ccccc32)cc(-n2c3ccccc3c3ccccc32)c1 ; N#Cc1cccc(-c2cc(-c3ccccc3-n3c4ccccc4c4ccccc43)cc(-n3c4ccccc4c4ccccc43)c2)c1 ; N#Cc1ccc(-c2cc(-c3ccccc3-n3c4ccccc4c4ccccc43)cc(-n3c4ccccc4c4ccccc43)c2)cc1]

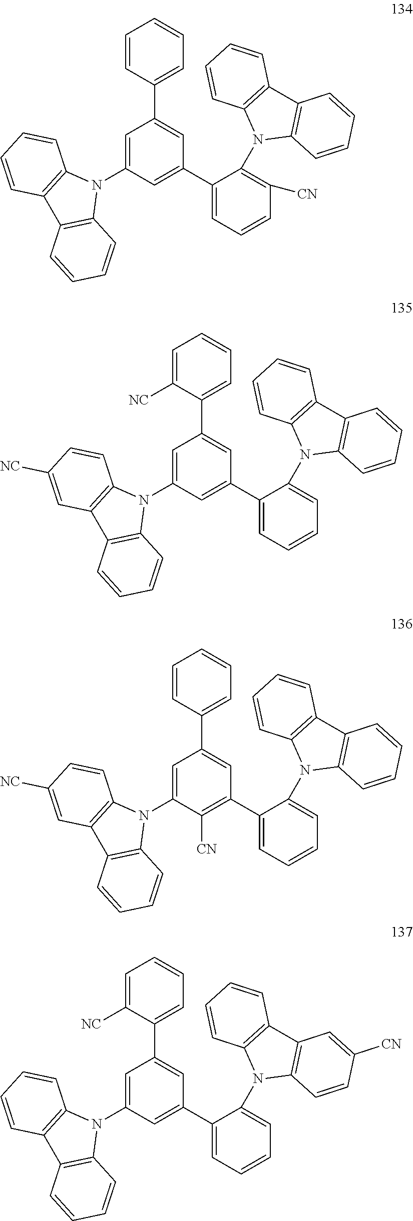 [SMILES: N#Cc1cccc(-c2cc(-c3ccccc3)cc(-n3c4ccccc4c4ccccc43)c2)c1-n1c2ccccc2c2ccccc21 ; N#Cc1ccc2c(c1)c1ccccc1n2-c1cc(-c2ccccc2C#N)cc(-c2ccccc2-n2c3ccccc3c3ccccc32)c1 ; N#Cc1ccc2c(c1)c1ccccc1n2-c1cc(-c2ccccc2)cc(-c2ccccc2-n2c3ccccc3c3ccccc32)c1C#N ; N#Cc1ccc2c(c1)c1ccccc1n2-c1ccccc1-c1cc(-c2ccccc2C#N)cc(-n2c3ccccc3c3ccccc32)c1]

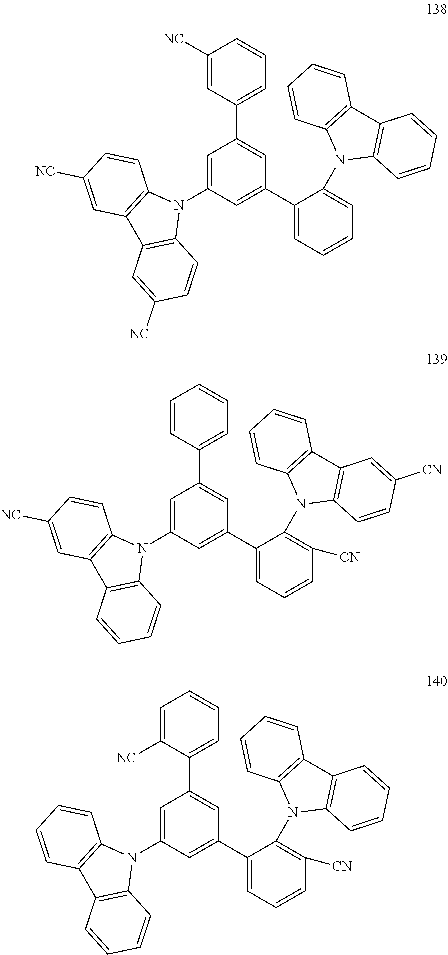 [SMILES: N#Cc1cccc(-c2cc(-c3ccccc3-n3c4ccccc4c4ccccc43)cc(-n3c4ccc(C#N)cc4c4cc(C#N)ccc43)c2)c1 ; N#Cc1ccc2c(c1)c1ccccc1n2-c1cc(-c2ccccc2)cc(-c2cccc(C#N)c2-n2c3ccccc3c3cc(C#N)ccc32)c1 ; N#Cc1ccccc1-c1cc(-c2cccc(C#N)c2-n2c3ccccc3c3ccccc32)cc(-n2c3ccccc3c3ccccc32)c1]

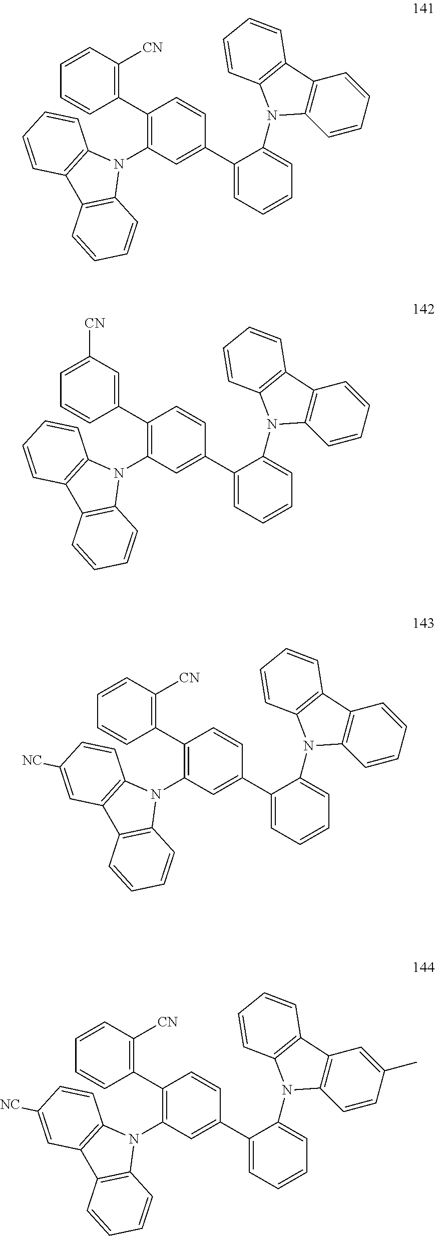

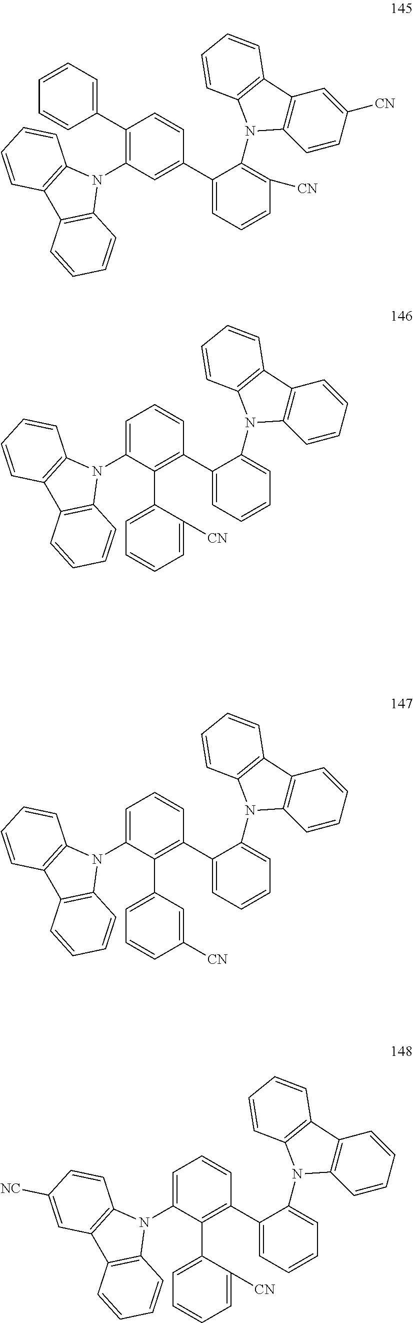 [SMILES: N#Cc1ccc2c(c1)c1ccccc1n2-c1c(C#N)cccc1-c1ccc(-c2ccccc2)c(-n2c3ccccc3c3ccccc32)c1 ; N#Cc1ccccc1-c1c(-c2ccccc2-n2c3ccccc3c3ccccc32)cccc1-n1c2ccccc2c2ccccc21 ; N#Cc1cccc(-c2c(-c3ccccc3-n3c4ccccc4c4ccccc43)cccc2-n2c3ccccc3c3ccccc32)c1 ; N#Cc1ccc2c(c1)c1ccccc1n2-c1cccc(-c2ccccc2-n2c3ccccc3c3ccccc32)c1-c1ccccc1C#N]

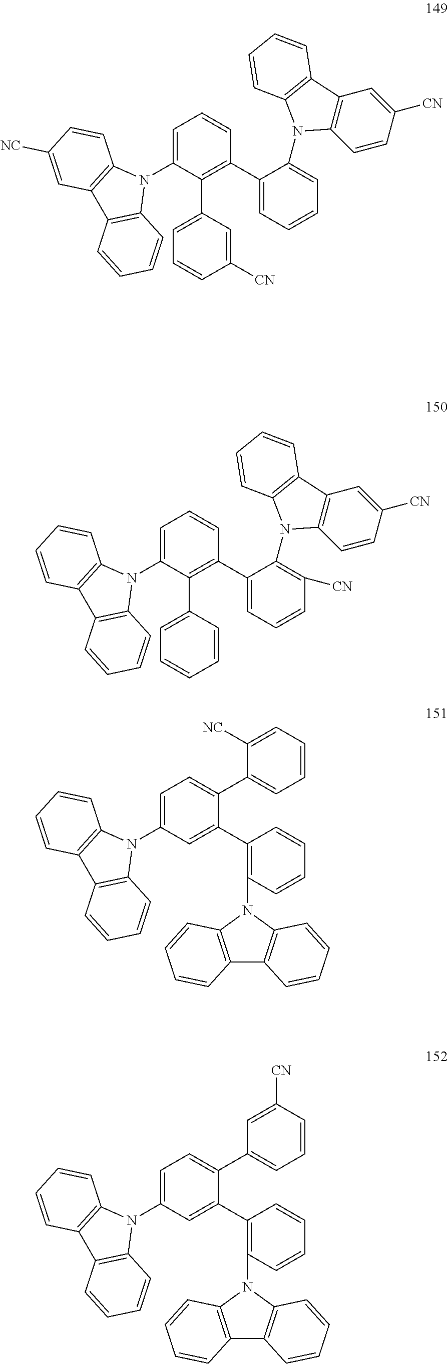 [SMILES: N#Cc1cccc(-c2c(-c3ccccc3-n3c4ccccc4c4cc(C#N)ccc43)cccc2-n2c3ccccc3c3cc(C#N)ccc32)c1 ; N#Cc1ccc2c(c1)c1ccccc1n2-c1c(C#N)cccc1-c1cccc(-n2c3ccccc3c3ccccc32)c1-c1ccccc1 ; N#Cc1ccccc1-c1ccc(-n2c3ccccc3c3ccccc32)cc1-c1ccccc1-n1c2ccccc2c2ccccc21 ; N#Cc1cccc(-c2ccc(-n3c4ccccc4c4ccccc43)cc2-c2ccccc2-n2c3ccccc3c3ccccc32)c1]

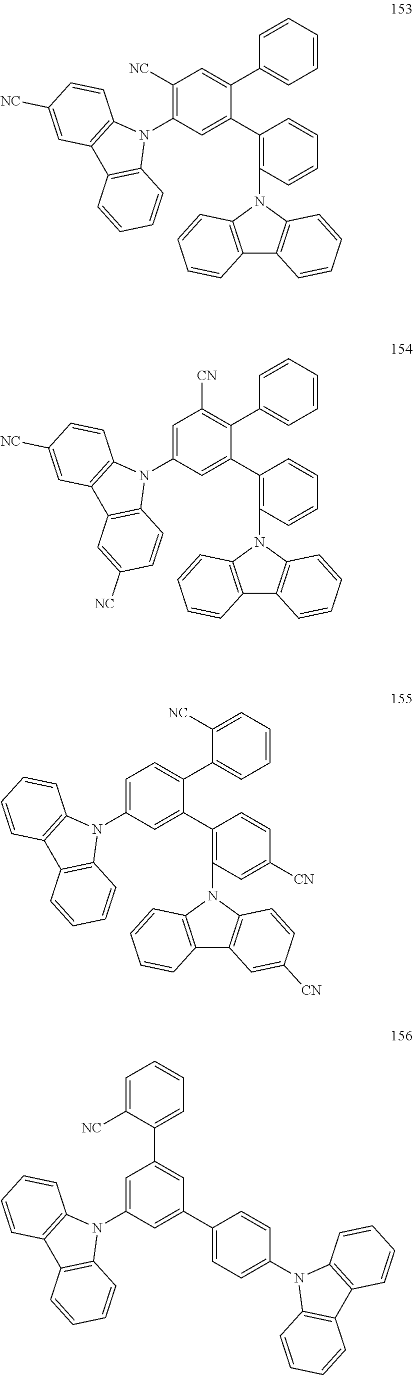 [SMILES: N#Cc1ccc2c(c1)c1ccccc1n2-c1cc(-c2ccccc2-n2c3ccccc3c3ccccc32)c(-c2ccccc2)cc1C#N ; N#Cc1ccc2c(c1)c1cc(C#N)ccc1n2-c1cc(C#N)c(-c2ccccc2)c(-c2ccccc2-n2c3ccccc3c3ccccc32)c1 ; N#Cc1ccc(-c2cc(-n3c4ccccc4c4ccccc43)ccc2-c2ccccc2C#N)c(-n2c3ccccc3c3cc(C#N)ccc32)c1 ; N#Cc1ccccc1-c1cc(-c2ccc(-n3c4ccccc4c4ccccc43)cc2)cc(-n2c3ccccc3c3ccccc32)c1]

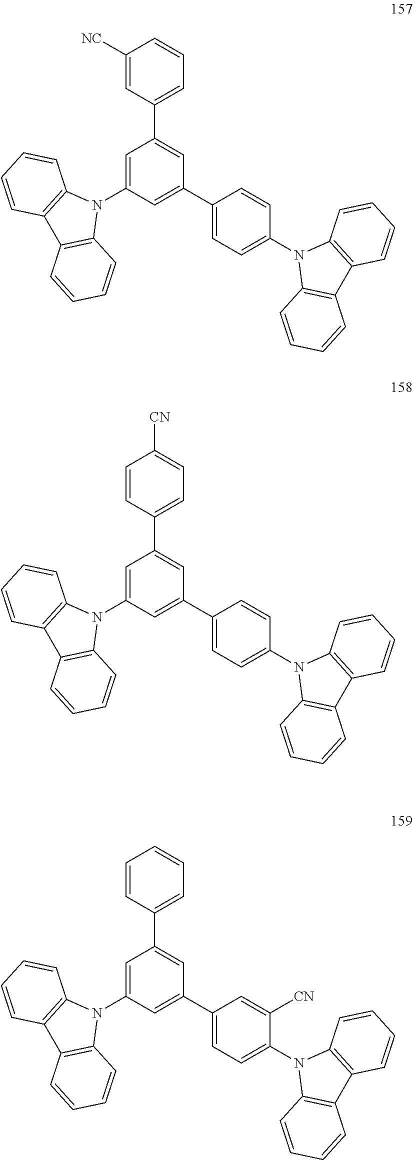 [SMILES: N#Cc1cccc(-c2cc(-c3ccc(-n4c5ccccc5c5ccccc54)cc3)cc(-n3c4ccccc4c4ccccc43)c2)c1 ; N#Cc1ccc(-c2cc(-c3ccc(-n4c5ccccc5c5ccccc54)cc3)cc(-n3c4ccccc4c4ccccc43)c2)cc1 ; N#Cc1cc(-c2cc(-c3ccccc3)cc(-n3c4ccccc4c4ccccc43)c2)ccc1-n1c2ccccc2c2ccccc21]

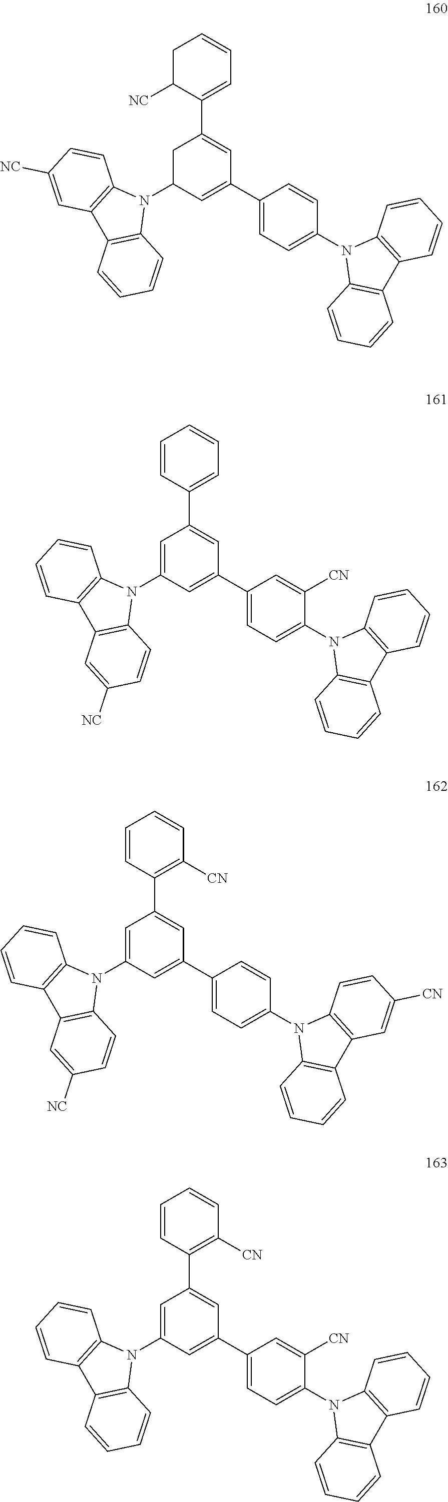 [SMILES: N#Cc1ccc2c(c1)c1ccccc1n2C1C=C(c2ccc(-n3c4ccccc4c4ccccc43)cc2)C=C(C2=CC=CCC2C#N)C1 ; N#Cc1ccc2c(c1)c1ccccc1n2-c1cc(-c2ccccc2)cc(-c2ccc(-n3c4ccccc4c4ccccc43)c(C#N)c2)c1 ; N#Cc1ccc2c(c1)c1ccccc1n2-c1ccc(-c2cc(-c3ccccc3C#N)cc(-n3c4ccccc4c4cc(C#N)ccc43)c2)cc1 ; N#Cc1ccccc1-c1cc(-c2ccc(-n3c4ccccc4c4ccccc43)c(C#N)c2)cc(-n2c3ccccc3c3ccccc32)c1]

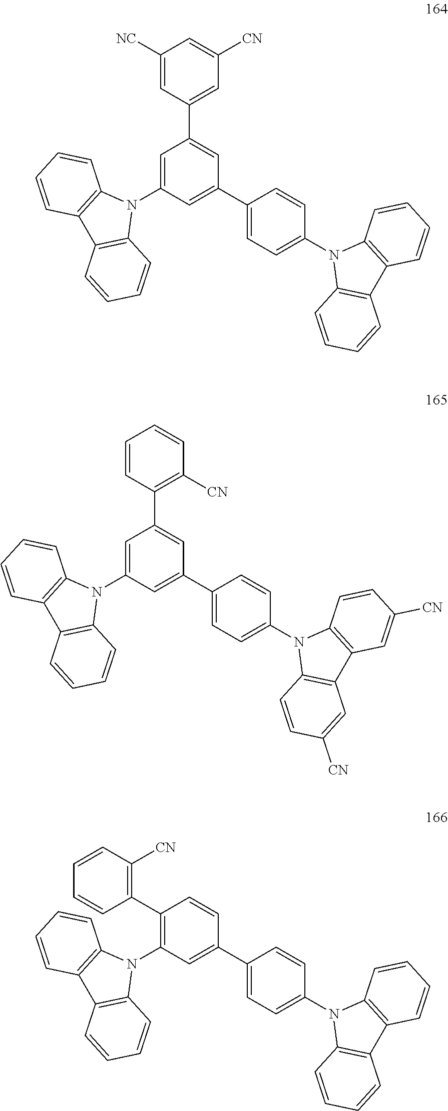 [SMILES: N#Cc1cc(C#N)cc(-c2cc(-c3ccc(-n4c5ccccc5c5ccccc54)cc3)cc(-n3c4ccccc4c4ccccc43)c2)c1 ; N#Cc1ccc2c(c1)c1cc(C#N)ccc1n2-c1ccc(-c2cc(-c3ccccc3C#N)cc(-n3c4ccccc4c4ccccc43)c2)cc1 ; N#Cc1ccccc1-c1ccc(-c2ccc(-n3c4ccccc4c4ccccc43)cc2)cc1-n1c2ccccc2c2ccccc21]

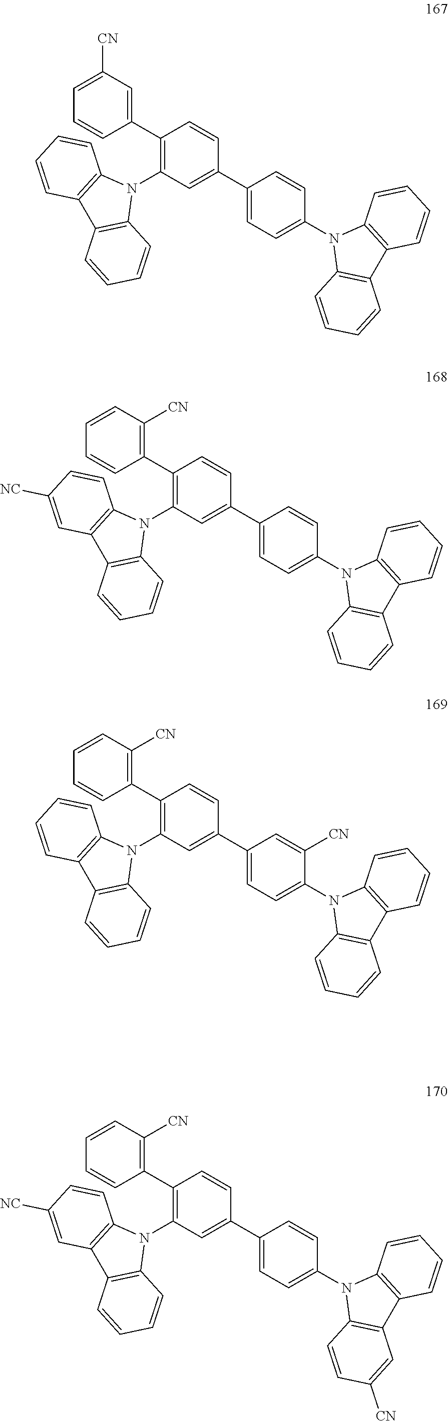 [SMILES: N#Cc1cccc(-c2ccc(-c3ccc(-n4c5ccccc5c5ccccc54)cc3)cc2-n2c3ccccc3c3ccccc32)c1 ; N#Cc1ccc2c(c1)c1ccccc1n2-c1cc(-c2ccc(-n3c4ccccc4c4ccccc43)cc2)ccc1-c1ccccc1C#N ; N#Cc1ccccc1-c1ccc(-c2ccc(-n3c4ccccc4c4ccccc43)c(C#N)c2)cc1-n1c2ccccc2c2ccccc21 ; N#Cc1ccc2c(c1)c1ccccc1n2-c1ccc(-c2ccc(-c3ccccc3C#N)c(-n3c4ccccc4c4cc(C#N)ccc43)c2)cc1]

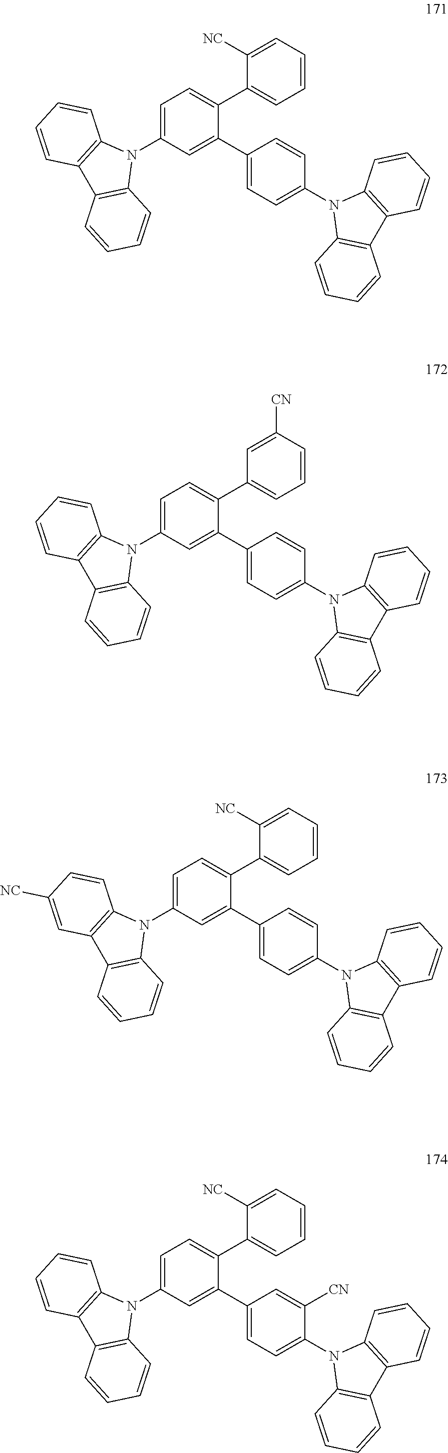 [SMILES: N#Cc1ccccc1-c1ccc(-n2c3ccccc3c3ccccc32)cc1-c1ccc(-n2c3ccccc3c3ccccc32)cc1 ; N#Cc1cccc(-c2ccc(-n3c4ccccc4c4ccccc43)cc2-c2ccc(-n3c4ccccc4c4ccccc43)cc2)c1 ; N#Cc1ccc2c(c1)c1ccccc1n2-c1ccc(-c2ccccc2C#N)c(-c2ccc(-n3c4ccccc4c4ccccc43)cc2)c1 ; N#Cc1ccccc1-c1ccc(-n2c3ccccc3c3ccccc32)cc1-c1ccc(-n2c3ccccc3c3ccccc32)c(C#N)c1]

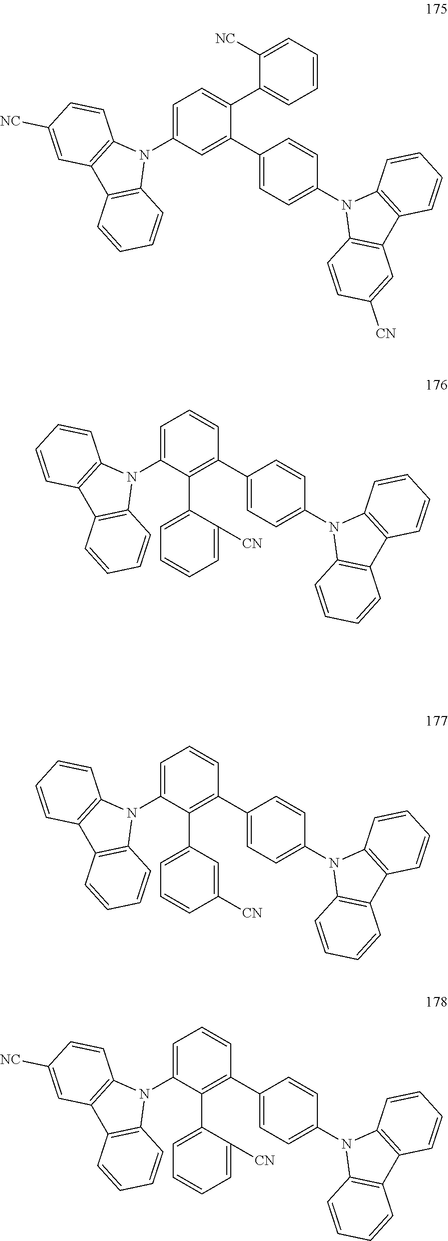 [SMILES: N#Cc1ccc2c(c1)c1ccccc1n2-c1ccc(-c2cc(-n3c4ccccc4c4cc(C#N)ccc43)ccc2-c2ccccc2C#N)cc1 ; N#Cc1ccccc1-c1c(-c2ccc(-n3c4ccccc4c4ccccc43)cc2)cccc1-n1c2ccccc2c2ccccc21 ; N#Cc1cccc(-c2c(-c3ccc(-n4c5ccccc5c5ccccc54)cc3)cccc2-n2c3ccccc3c3ccccc32)c1 ; N#Cc1ccc2c(c1)c1ccccc1n2-c1cccc(-c2ccc(-n3c4ccccc4c4ccccc43)cc2)c1-c1ccccc1C#N]

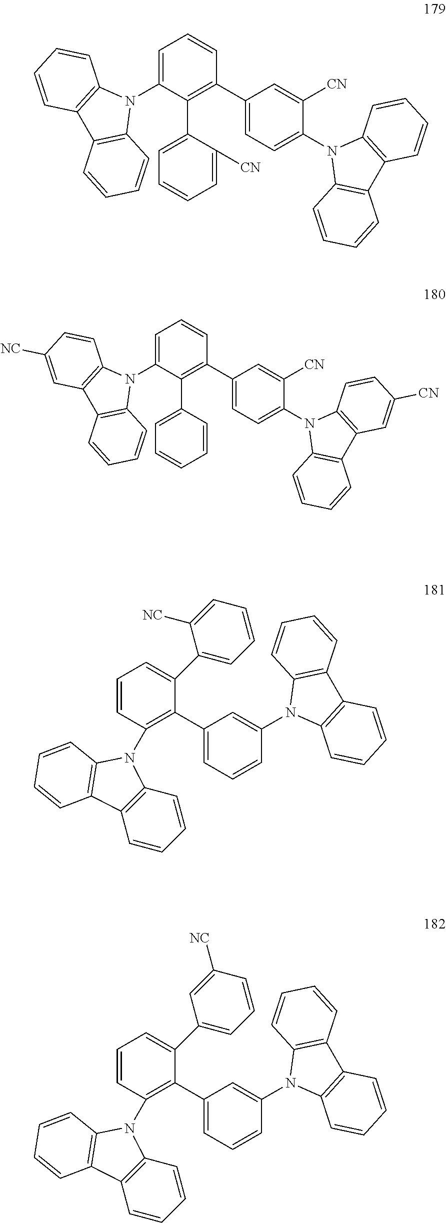 [SMILES: N#Cc1ccccc1-c1c(-c2ccc(-n3c4ccccc4c4ccccc43)c(C#N)c2)cccc1-n1c2ccccc2c2ccccc21 ; N#Cc1ccc2c(c1)c1ccccc1n2-c1ccc(-c2cccc(-n3c4ccccc4c4cc(C#N)ccc43)c2-c2ccccc2)cc1C#N ; N#Cc1ccccc1-c1cccc(-n2c3ccccc3c3ccccc32)c1-c1cccc(-n2c3ccccc3c3ccccc32)c1 ; N#Cc1cccc(-c2cccc(-n3c4ccccc4c4ccccc43)c2-c2cccc(-n3c4ccccc4c4ccccc43)c2)c1]

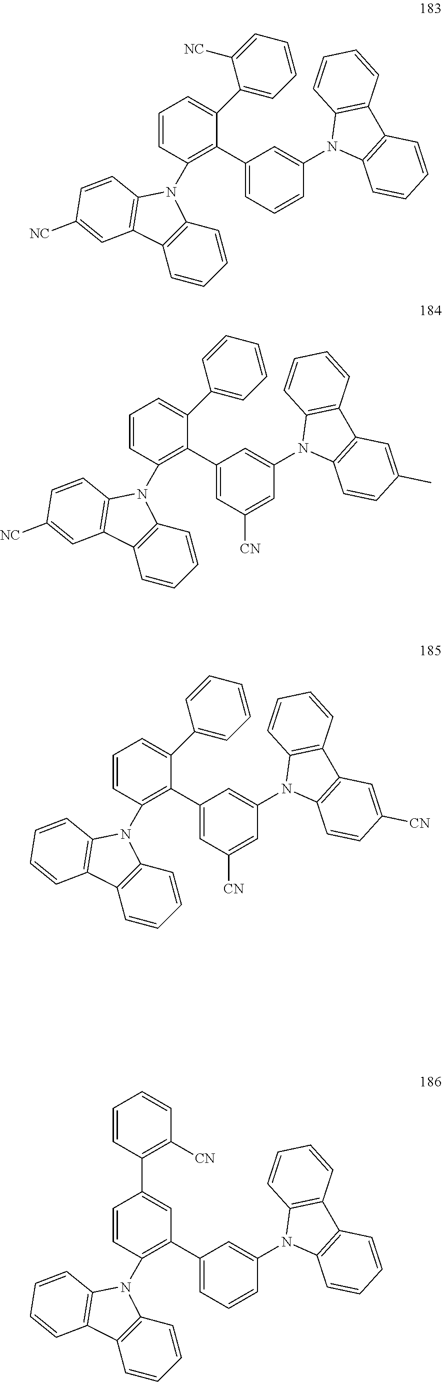 [SMILES: N#Cc1ccc2c(c1)c1ccccc1n2-c1cccc(-c2ccccc2C#N)c1-c1cccc(-n2c3ccccc3c3ccccc32)c1 ; Cc1ccc2c(c1)c1ccccc1n2-c1cc(C#N)cc(-c2c(-c3ccccc3)cccc2-n2c3ccccc3c3cc(C#N)ccc32)c1 ; N#Cc1cc(-c2c(-c3ccccc3)cccc2-n2c3ccccc3c3ccccc32)cc(-n2c3ccccc3c3cc(C#N)ccc32)c1 ; N#Cc1ccccc1-c1ccc(-n2c3ccccc3c3ccccc32)c(-c2cccc(-n3c4ccccc4c4ccccc43)c2)c1]

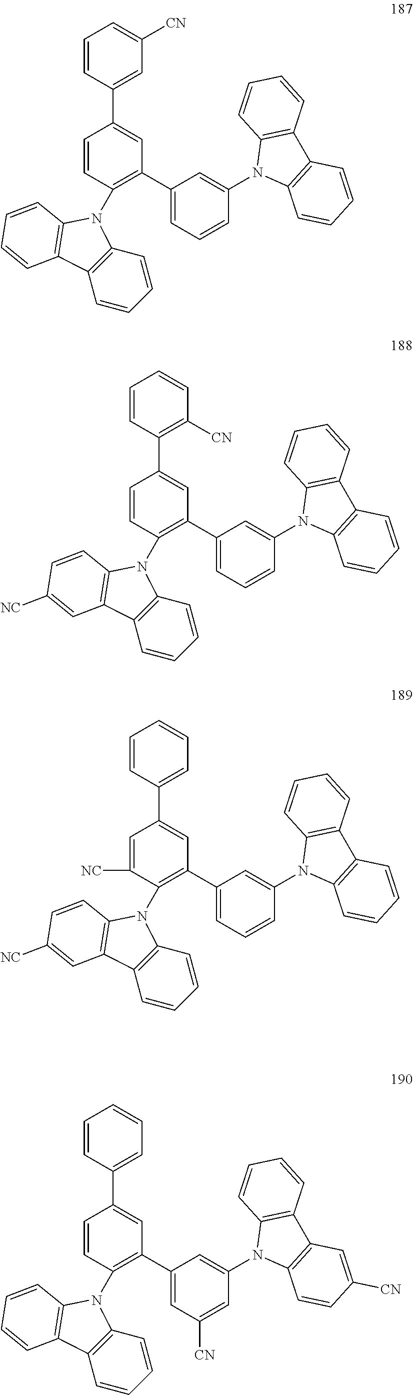 [SMILES: N#Cc1cccc(-c2ccc(-n3c4ccccc4c4ccccc43)c(-c3cccc(-n4c5ccccc5c5ccccc54)c3)c2)c1 ; N#Cc1ccc2c(c1)c1ccccc1n2-c1ccc(-c2ccccc2C#N)cc1-c1cccc(-n2c3ccccc3c3ccccc32)c1 ; N#Cc1ccc2c(c1)c1ccccc1n2-c1c(C#N)cc(-c2ccccc2)cc1-c1cccc(-n2c3ccccc3c3ccccc32)c1 ; N#Cc1cc(-c2cc(-c3ccccc3)ccc2-n2c3ccccc3c3ccccc32)cc(-n2c3ccccc3c3cc(C#N)ccc32)c1]

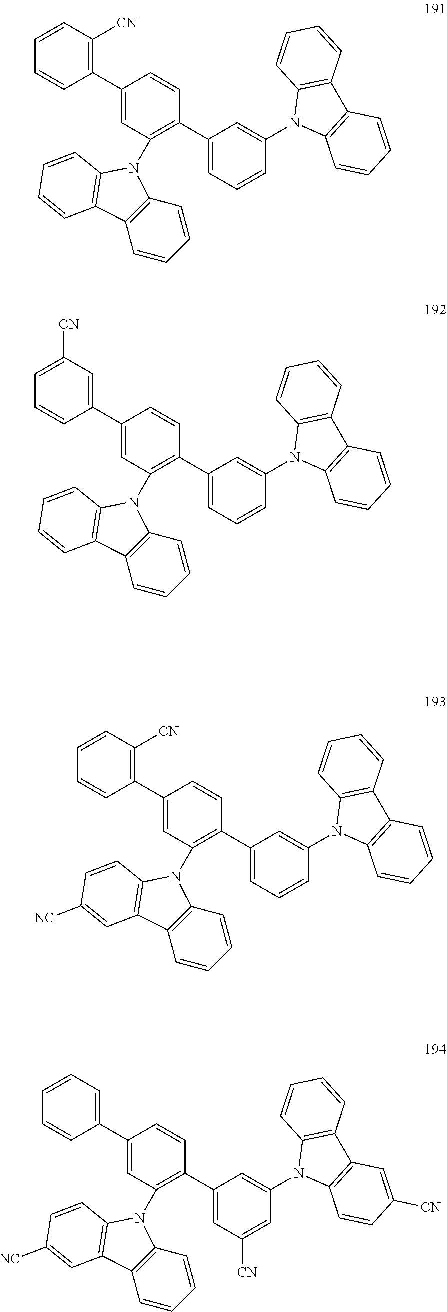 [SMILES: N#Cc1ccccc1-c1ccc(-c2cccc(-n3c4ccccc4c4ccccc43)c2)c(-n2c3ccccc3c3ccccc32)c1 ; N#Cc1cccc(-c2ccc(-c3cccc(-n4c5ccccc5c5ccccc54)c3)c(-n3c4ccccc4c4ccccc43)c2)c1 ; N#Cc1ccc2c(c1)c1ccccc1n2-c1cc(-c2ccccc2C#N)ccc1-c1cccc(-n2c3ccccc3c3ccccc32)c1 ; N#Cc1cc(-c2ccc(-c3ccccc3)cc2-n2c3ccccc3c3cc(C#N)ccc32)cc(-n2c3ccccc3c3cc(C#N)ccc32)c1]

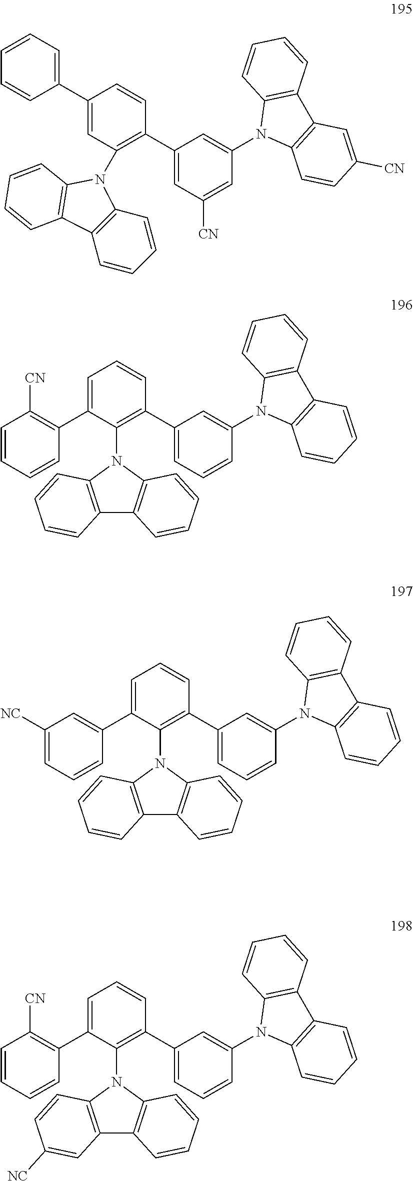 [SMILES: N#Cc1cc(-c2ccc(-c3ccccc3)cc2-n2c3ccccc3c3ccccc32)cc(-n2c3ccccc3c3cc(C#N)ccc32)c1 ; N#Cc1ccccc1-c1cccc(-c2cccc(-n3c4ccccc4c4ccccc43)c2)c1-n1c2ccccc2c2ccccc21 ; N#Cc1cccc(-c2cccc(-c3cccc(-n4c5ccccc5c5ccccc54)c3)c2-n2c3ccccc3c3ccccc32)c1 ; N#Cc1ccc2c(c1)c1ccccc1n2-c1c(-c2cccc(-n3c4ccccc4c4ccccc43)c2)cccc1-c1ccccc1C#N]

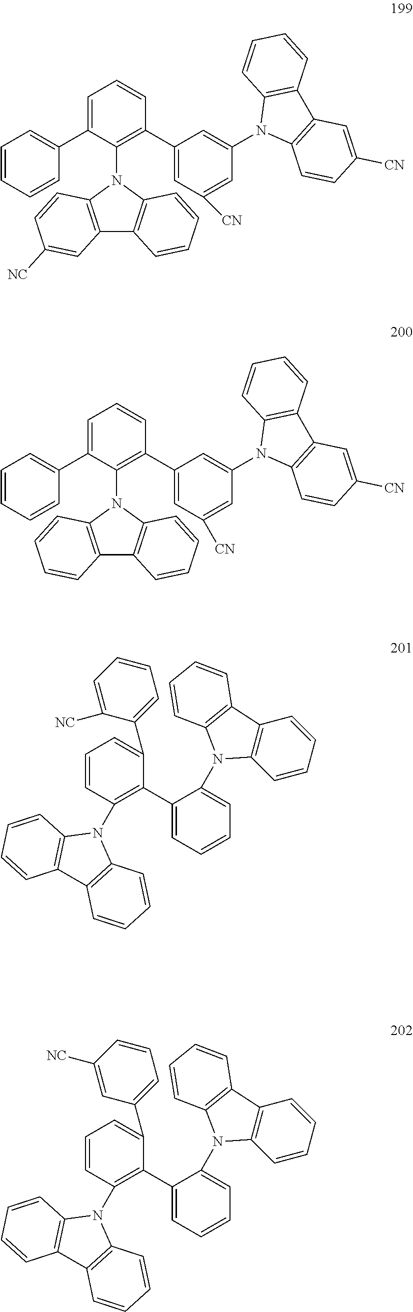 [SMILES: N#Cc1cc(-c2cccc(-c3ccccc3)c2-n2c3ccccc3c3cc(C#N)ccc32)cc(-n2c3ccccc3c3cc(C#N)ccc32)c1 ; N#Cc1cc(-c2cccc(-c3ccccc3)c2-n2c3ccccc3c3ccccc32)cc(-n2c3ccccc3c3cc(C#N)ccc32)c1 ; N#Cc1ccccc1-c1cccc(-n2c3ccccc3c3ccccc32)c1-c1ccccc1-n1c2ccccc2c2ccccc21 ; N#Cc1cccc(-c2cccc(-n3c4ccccc4c4ccccc43)c2-c2ccccc2-n2c3ccccc3c3ccccc32)c1]

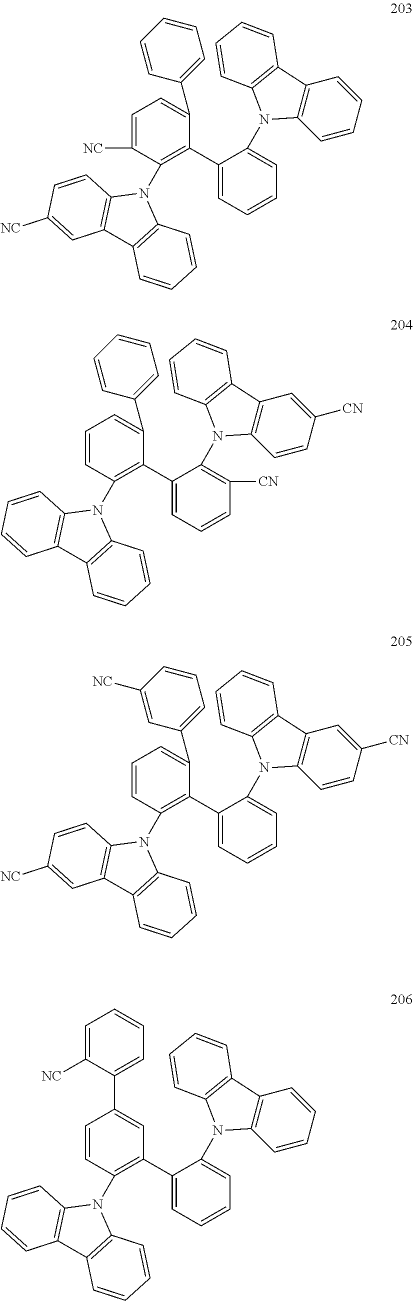 [SMILES: N#Cc1ccc2c(c1)c1ccccc1n2-c1c(C#N)ccc(-c2ccccc2)c1-c1ccccc1-n1c2ccccc2c2ccccc21 ; N#Cc1ccc2c(c1)c1ccccc1n2-c1c(C#N)cccc1-c1c(-c2ccccc2)cccc1-n1c2ccccc2c2ccccc21 ; N#Cc1cccc(-c2cccc(-n3c4ccccc4c4cc(C#N)ccc43)c2-c2ccccc2-n2c3ccccc3c3cc(C#N)ccc32)c1 ; N#Cc1ccccc1-c1ccc(-n2c3ccccc3c3ccccc32)c(-c2ccccc2-n2c3ccccc3c3ccccc32)c1]

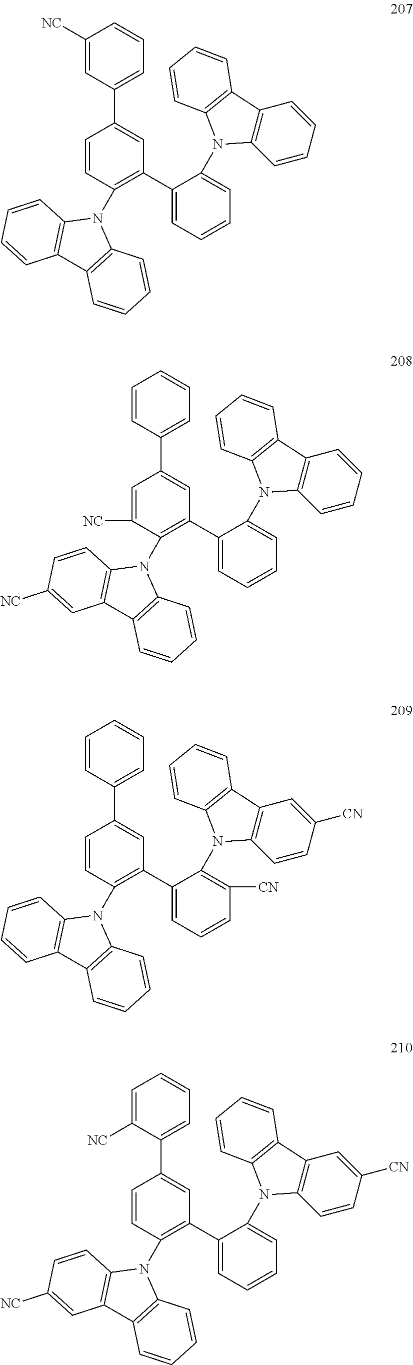 [SMILES: N#Cc1cccc(-c2ccc(-n3c4ccccc4c4ccccc43)c(-c3ccccc3-n3c4ccccc4c4ccccc43)c2)c1 ; N#Cc1ccc2c(c1)c1ccccc1n2-c1c(C#N)cc(-c2ccccc2)cc1-c1ccccc1-n1c2ccccc2c2ccccc21 ; N#Cc1ccc2c(c1)c1ccccc1n2-c1c(C#N)cccc1-c1cc(-c2ccccc2)ccc1-n1c2ccccc2c2ccccc21 ; N#Cc1ccc2c(c1)c1ccccc1n2-c1ccccc1-c1cc(-c2ccccc2C#N)ccc1-n1c2ccccc2c2cc(C#N)ccc21]

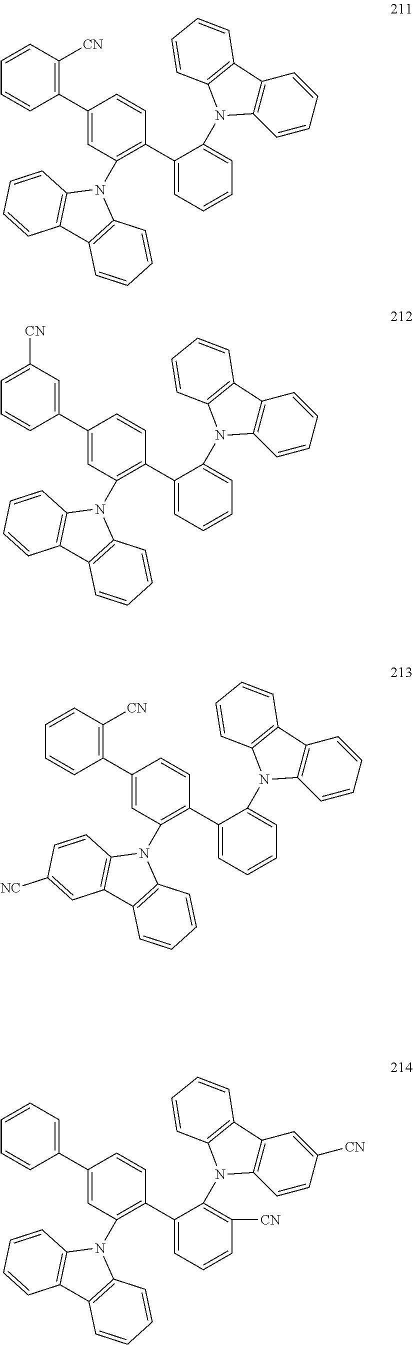 [SMILES: N#Cc1ccccc1-c1ccc(-c2ccccc2-n2c3ccccc3c3ccccc32)c(-n2c3ccccc3c3ccccc32)c1 ; N#Cc1cccc(-c2ccc(-c3ccccc3-n3c4ccccc4c4ccccc43)c(-n3c4ccccc4c4ccccc43)c2)c1 ; N#Cc1ccc2c(c1)c1ccccc1n2-c1cc(-c2ccccc2C#N)ccc1-c1ccccc1-n1c2ccccc2c2ccccc21 ; N#Cc1ccc2c(c1)c1ccccc1n2-c1c(C#N)cccc1-c1ccc(-c2ccccc2)cc1-n1c2ccccc2c2ccccc21]

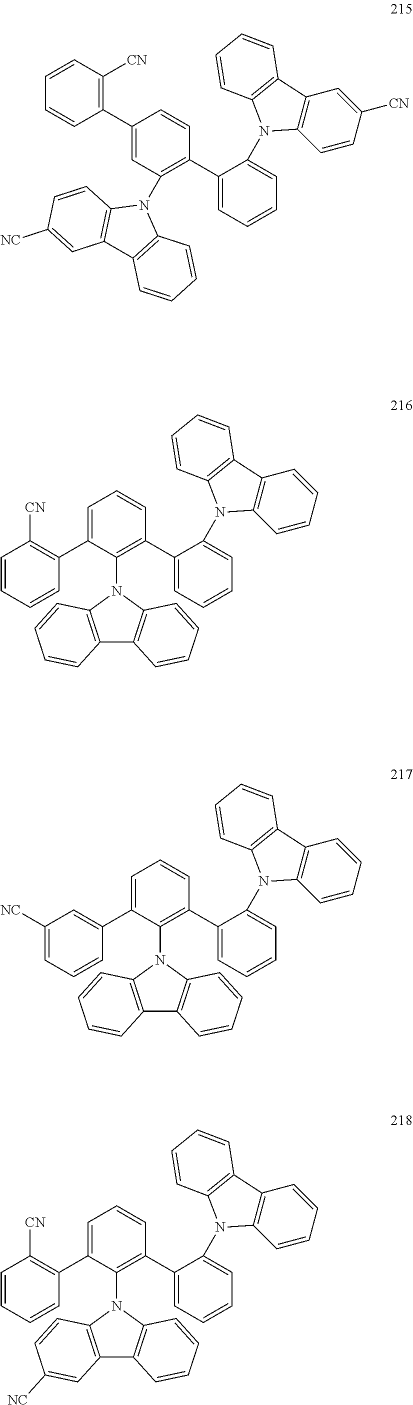 [SMILES: N#Cc1ccc2c(c1)c1ccccc1n2-c1ccccc1-c1ccc(-c2ccccc2C#N)cc1-n1c2ccccc2c2cc(C#N)ccc21 ; N#Cc1ccccc1-c1cccc(-c2ccccc2-n2c3ccccc3c3ccccc32)c1-n1c2ccccc2c2ccccc21 ; N#Cc1cccc(-c2cccc(-c3ccccc3-n3c4ccccc4c4ccccc43)c2-n2c3ccccc3c3ccccc32)c1 ; N#Cc1ccc2c(c1)c1ccccc1n2-c1c(-c2ccccc2C#N)cccc1-c1ccccc1-n1c2ccccc2c2ccccc21]

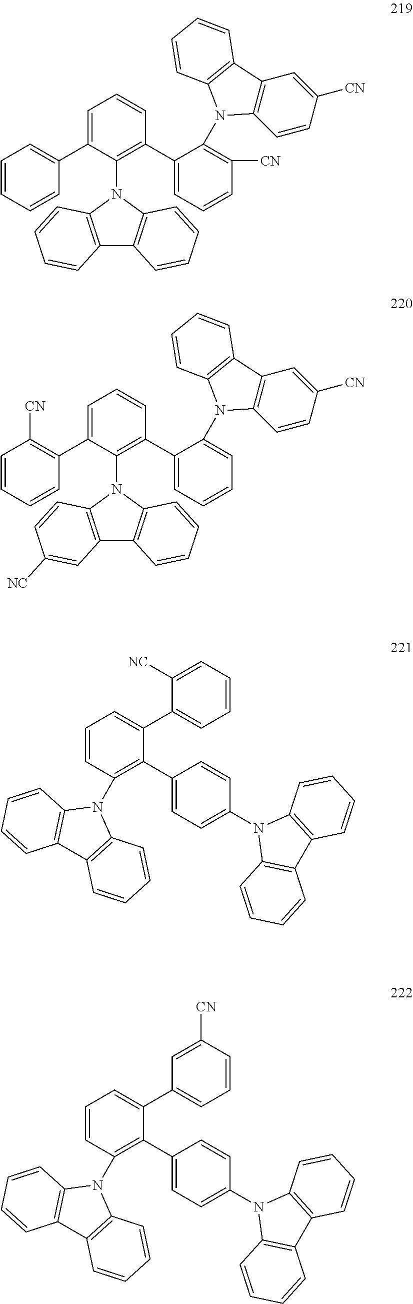 [SMILES: N#Cc1ccc2c(c1)c1ccccc1n2-c1c(C#N)cccc1-c1cccc(-c2ccccc2)c1-n1c2ccccc2c2ccccc21 ; N#Cc1ccc2c(c1)c1ccccc1n2-c1ccccc1-c1cccc(-c2ccccc2C#N)c1-n1c2ccccc2c2cc(C#N)ccc21 ; N#Cc1ccccc1-c1cccc(-n2c3ccccc3c3ccccc32)c1-c1ccc(-n2c3ccccc3c3ccccc32)cc1 ; N#Cc1cccc(-c2cccc(-n3c4ccccc4c4ccccc43)c2-c2ccc(-n3c4ccccc4c4ccccc43)cc2)c1]

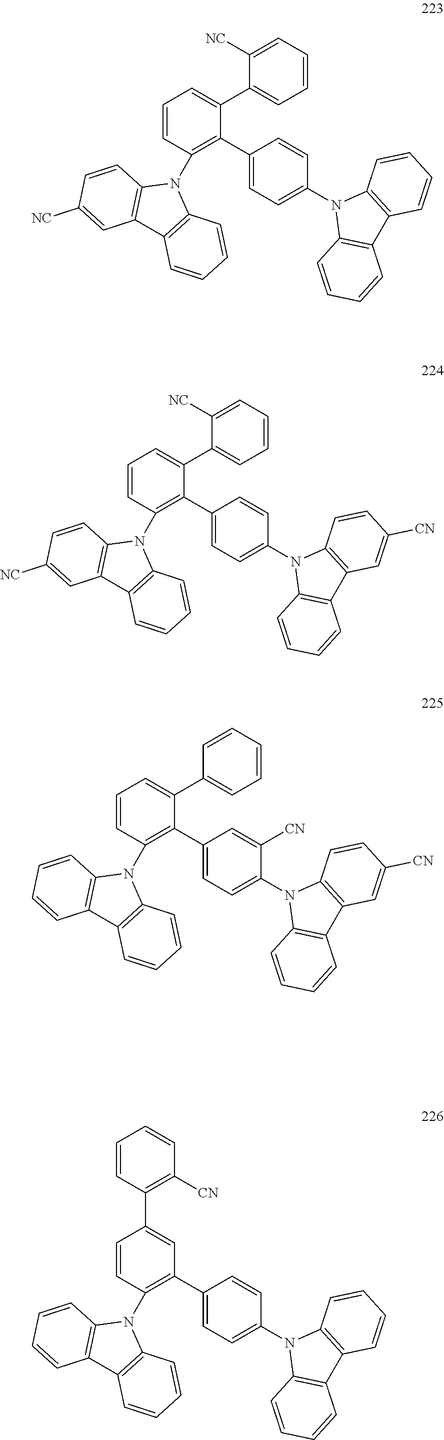 [SMILES: N#Cc1ccc2c(c1)c1ccccc1n2-c1cccc(-c2ccccc2C#N)c1-c1ccc(-n2c3ccccc3c3ccccc32)cc1 ; N#Cc1ccc2c(c1)c1ccccc1n2-c1ccc(-c2c(-c3ccccc3C#N)cccc2-n2c3ccccc3c3cc(C#N)ccc32)cc1 ; N#Cc1ccc2c(c1)c1ccccc1n2-c1ccc(-c2c(-c3ccccc3)cccc2-n2c3ccccc3c3ccccc32)cc1C#N ; N#Cc1ccccc1-c1ccc(-n2c3ccccc3c3ccccc32)c(-c2ccc(-n3c4ccccc4c4ccccc43)cc2)c1]

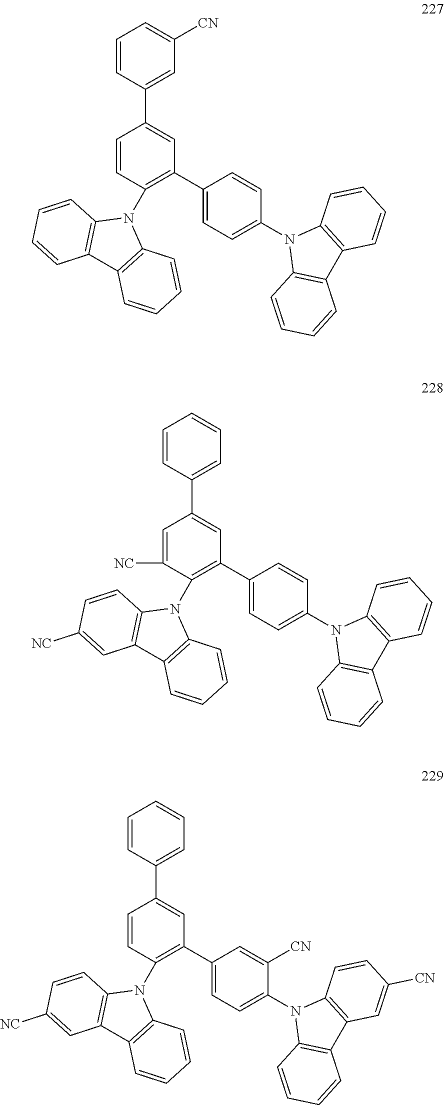 [SMILES: N#Cc1cccc(-c2ccc(-n3c4ccccc4c4ccccc43)c(-c3ccc(-n4c5ccccc5c5ccccc54)cc3)c2)c1 ; N#Cc1ccc2c(c1)c1ccccc1n2-c1c(C#N)cc(-c2ccccc2)cc1-c1ccc(-n2c3ccccc3c3ccccc32)cc1 ; N#Cc1ccc2c(c1)c1ccccc1n2-c1ccc(-c2cc(-c3ccccc3)ccc2-n2c3ccccc3c3cc(C#N)ccc32)cc1C#N]

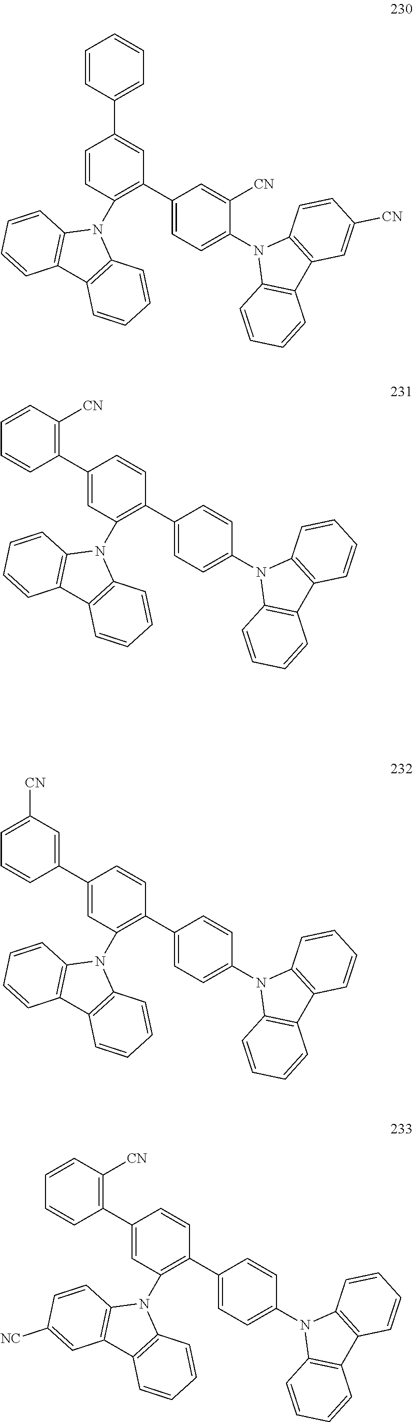 [SMILES: N#Cc1ccc2c(c1)c1ccccc1n2-c1ccc(-c2cc(-c3ccccc3)ccc2-n2c3ccccc3c3ccccc32)cc1C#N ; N#Cc1ccccc1-c1ccc(-c2ccc(-n3c4ccccc4c4ccccc43)cc2)c(-n2c3ccccc3c3ccccc32)c1 ; N#Cc1cccc(-c2ccc(-c3ccc(-n4c5ccccc5c5ccccc54)cc3)c(-n3c4ccccc4c4ccccc43)c2)c1 ; N#Cc1ccc2c(c1)c1ccccc1n2-c1cc(-c2ccccc2C#N)ccc1-c1ccc(-n2c3ccccc3c3ccccc32)cc1]

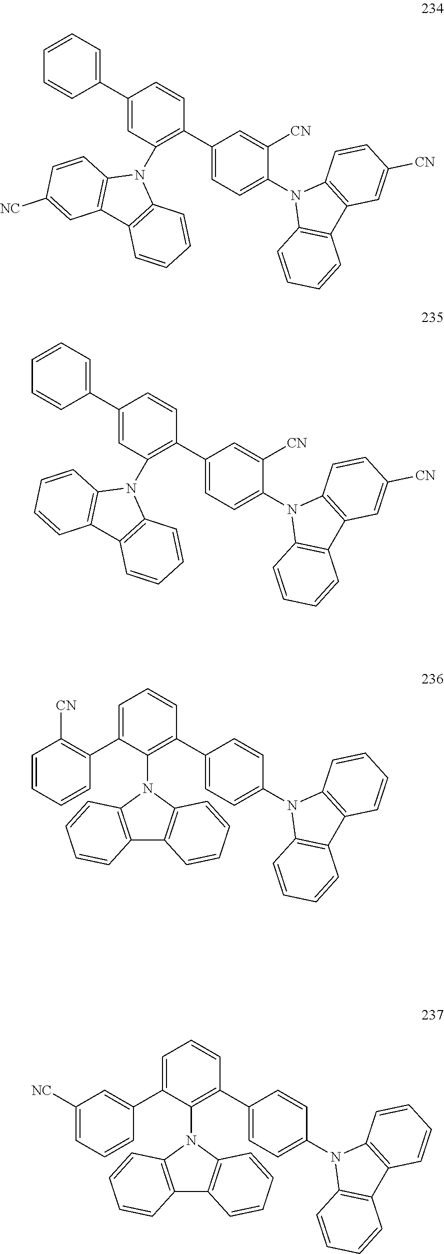 [SMILES: N#Cc1ccc2c(c1)c1ccccc1n2-c1ccc(-c2ccc(-c3ccccc3)cc2-n2c3ccccc3c3cc(C#N)ccc32)cc1C#N ; N#Cc1ccc2c(c1)c1ccccc1n2-c1ccc(-c2ccc(-c3ccccc3)cc2-n2c3ccccc3c3ccccc32)cc1C#N ; N#Cc1ccccc1-c1cccc(-c2ccc(-n3c4ccccc4c4ccccc43)cc2)c1-n1c2ccccc2c2ccccc21 ; N#Cc1cccc(-c2cccc(-c3ccc(-n4c5ccccc5c5ccccc54)cc3)c2-n2c3ccccc3c3ccccc32)c1]

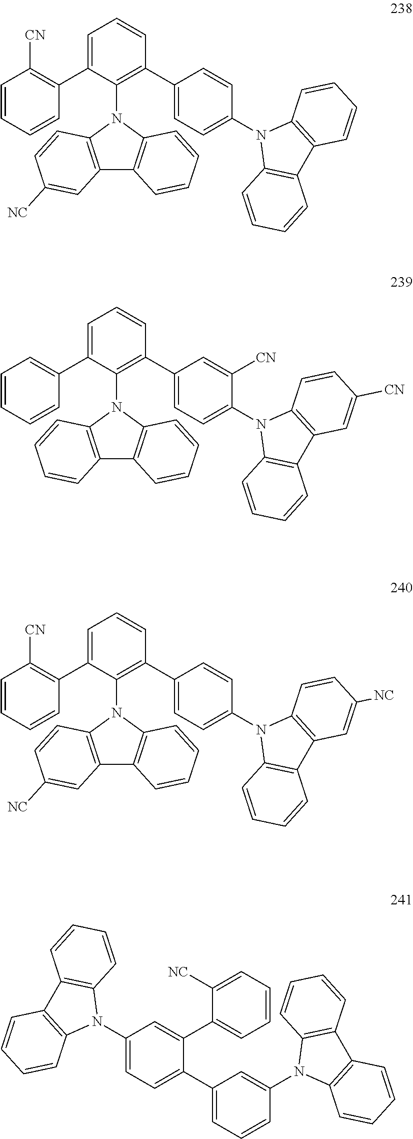 [SMILES: N#Cc1ccc2c(c1)c1ccccc1n2-c1c(-c2ccc(-n3c4ccccc4c4ccccc43)cc2)cccc1-c1ccccc1C#N ; N#Cc1ccc2c(c1)c1ccccc1n2-c1ccc(-c2cccc(-c3ccccc3)c2-n2c3ccccc3c3ccccc32)cc1C#N ; N#Cc1ccc2c(c1)c1ccccc1n2-c1ccc(-c2cccc(-c3ccccc3C#N)c2-n2c3ccccc3c3cc(C#N)ccc32)cc1 ; N#Cc1ccccc1-c1cc(-n2c3ccccc3c3ccccc32)ccc1-c1cccc(-n2c3ccccc3c3ccccc32)c1]

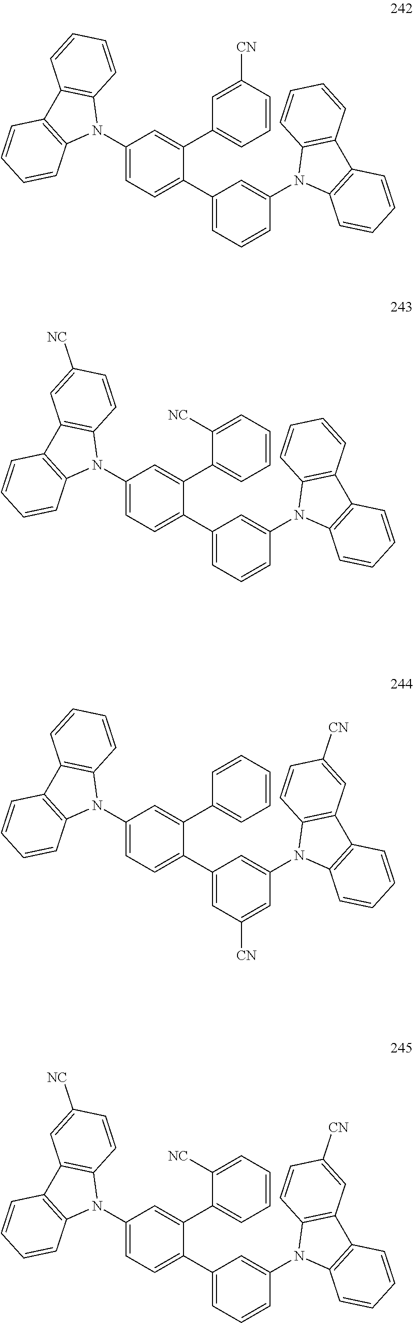 [SMILES: N#Cc1cccc(-c2cc(-n3c4ccccc4c4ccccc43)ccc2-c2cccc(-n3c4ccccc4c4ccccc43)c2)c1 ; N#Cc1ccc2c(c1)c1ccccc1n2-c1ccc(-c2cccc(-n3c4ccccc4c4ccccc43)c2)c(-c2ccccc2C#N)c1 ; N#Cc1cc(-c2ccc(-n3c4ccccc4c4ccccc43)cc2-c2ccccc2)cc(-n2c3ccccc3c3cc(C#N)ccc32)c1 ; N#Cc1ccc2c(c1)c1ccccc1n2-c1cccc(-c2ccc(-n3c4ccccc4c4cc(C#N)ccc43)cc2-c2ccccc2C#N)c1]

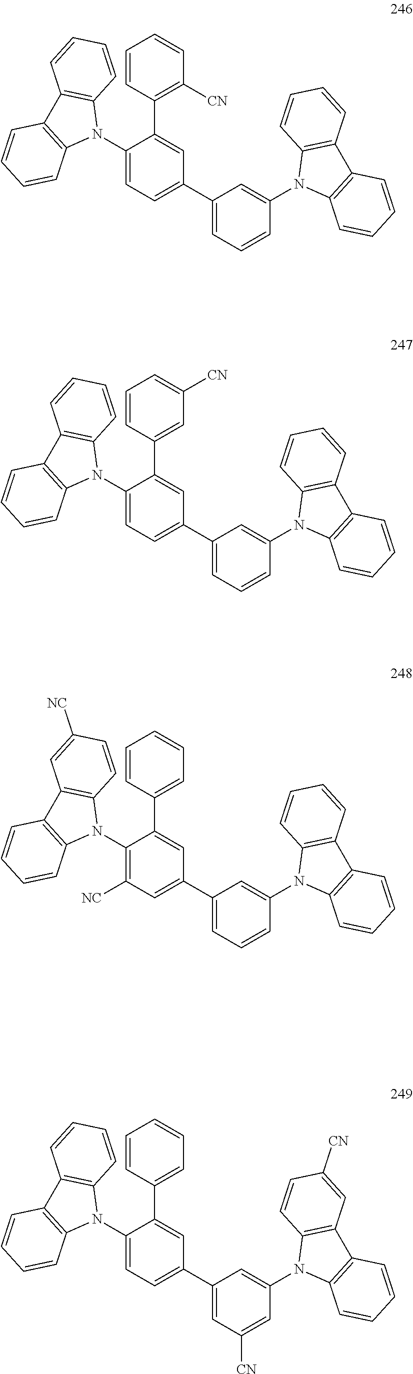 [SMILES: N#Cc1ccccc1-c1cc(-c2cccc(-n3c4ccccc4c4ccccc43)c2)ccc1-n1c2ccccc2c2ccccc21 ; N#Cc1cccc(-c2cc(-c3cccc(-n4c5ccccc5c5ccccc54)c3)ccc2-n2c3ccccc3c3ccccc32)c1 ; N#Cc1ccc2c(c1)c1ccccc1n2-c1c(C#N)cc(-c2cccc(-n3c4ccccc4c4ccccc43)c2)cc1-c1ccccc1 ; N#Cc1cc(-c2ccc(-n3c4ccccc4c4ccccc43)c(-c3ccccc3)c2)cc(-n2c3ccccc3c3cc(C#N)ccc32)c1]

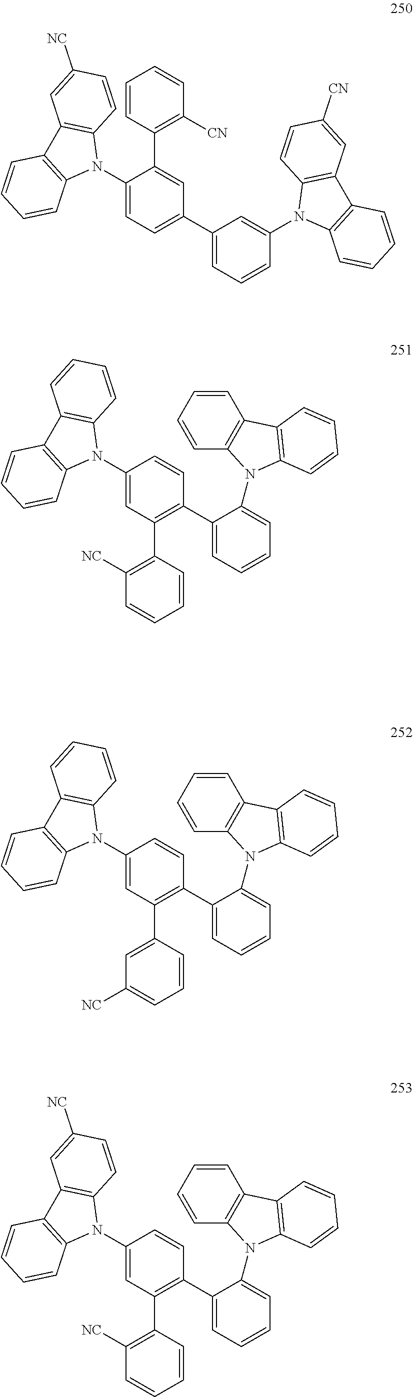 [SMILES: N#Cc1ccc2c(c1)c1ccccc1n2-c1cccc(-c2ccc(-n3c4ccccc4c4cc(C#N)ccc43)c(-c3ccccc3C#N)c2)c1 ; N#Cc1ccccc1-c1cc(-n2c3ccccc3c3ccccc32)ccc1-c1ccccc1-n1c2ccccc2c2ccccc21 ; N#Cc1cccc(-c2cc(-n3c4ccccc4c4ccccc43)ccc2-c2ccccc2-n2c3ccccc3c3ccccc32)c1 ; N#Cc1ccc2c(c1)c1ccccc1n2-c1ccc(-c2ccccc2-n2c3ccccc3c3ccccc32)c(-c2ccccc2C#N)c1]

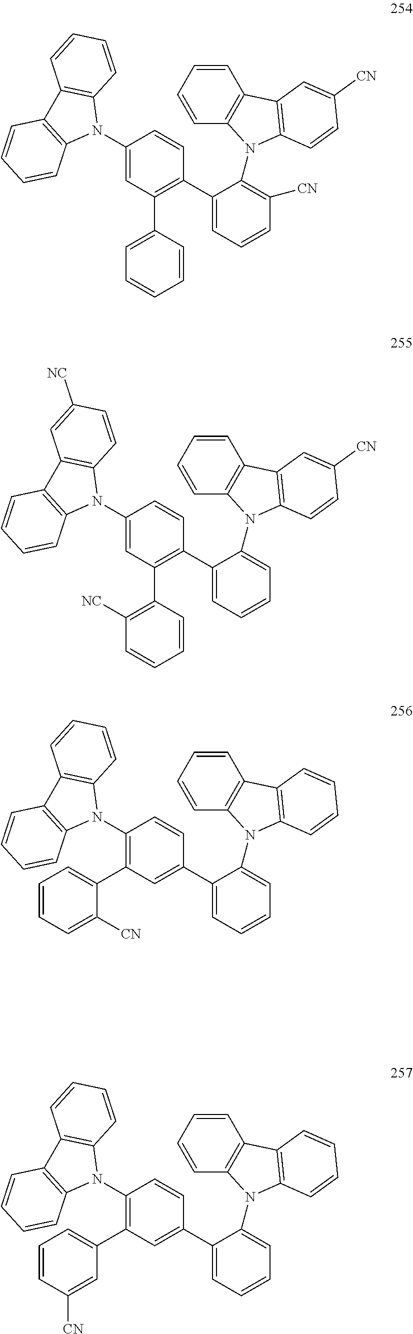 [SMILES: N#Cc1ccc2c(c1)c1ccccc1n2-c1c(C#N)cccc1-c1ccc(-n2c3ccccc3c3ccccc32)cc1-c1ccccc1 ; N#Cc1ccc2c(c1)c1ccccc1n2-c1ccc(-c2ccccc2-n2c3ccccc3c3cc(C#N)ccc32)c(-c2ccccc2C#N)c1 ; N#Cc1ccccc1-c1cc(-c2ccccc2-n2c3ccccc3c3ccccc32)ccc1-n1c2ccccc2c2ccccc21 ; N#Cc1cccc(-c2cc(-c3ccccc3-n3c4ccccc4c4ccccc43)ccc2-n2c3ccccc3c3ccccc32)c1]

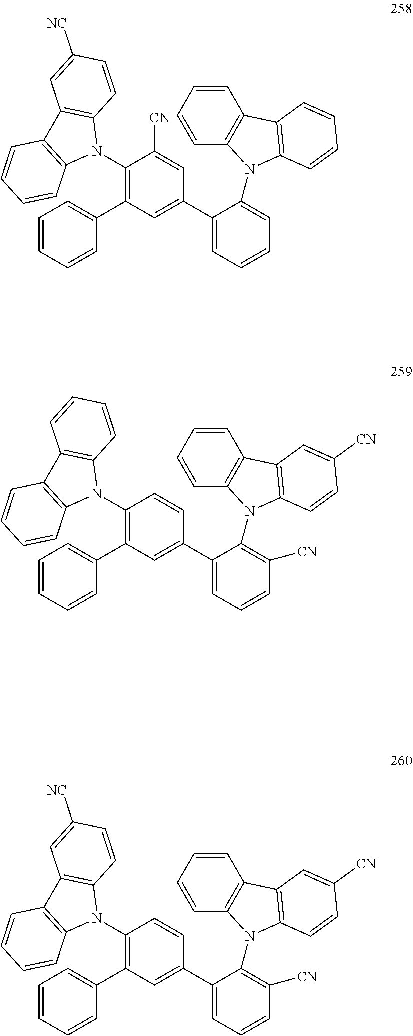 [SMILES: N#Cc1ccc2c(c1)c1ccccc1n2-c1c(C#N)cc(-c2ccccc2-n2c3ccccc3c3ccccc32)cc1-c1ccccc1 ; N#Cc1ccc2c(c1)c1ccccc1n2-c1c(C#N)cccc1-c1ccc(-n2c3ccccc3c3ccccc32)c(-c2ccccc2)c1 ; N#Cc1ccc2c(c1)c1ccccc1n2-c1ccc(-c2cccc(C#N)c2-n2c3ccccc3c3cc(C#N)ccc32)cc1-c1ccccc1]

In one or more embodiments, the electron-transport host may include DPEPO and TSPO1:

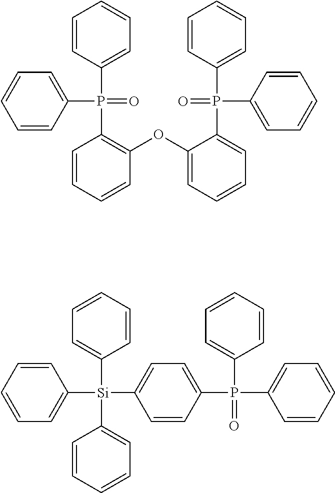 [SMILES: O=P(c1ccccc1)(c1ccccc1)c1ccccc1Oc1ccccc1P(=O)(c1ccccc1)c1ccccc1 ; O=P(c1ccccc1)(c1ccccc1)c1ccc([Si](c2ccccc2)(c2ccccc2)c2ccccc2)cc1]

In one or more embodiments, the hole-transporting host may be compounds of Group HH1, but embodiments of the present disclosure are not limited thereto:

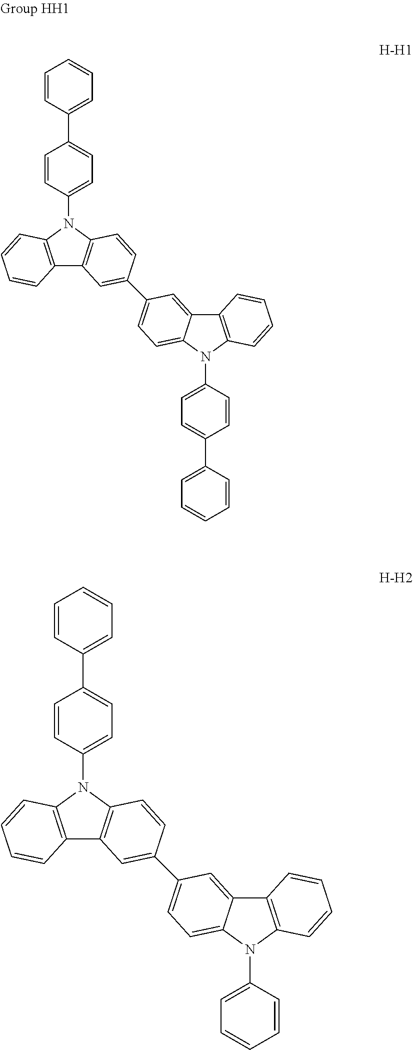 [SMILES: c1ccc(-c2ccc(-n3c4ccccc4c4cc(-c5ccc6c(c5)c5ccccc5n6-c5ccc(-c6ccccc6)cc5)ccc43)cc2)cc1 ; c1ccc(-c2ccc(-n3c4ccccc4c4cc(-c5ccc6c(c5)c5ccccc5n6-c5ccccc5)ccc43)cc2)cc1]

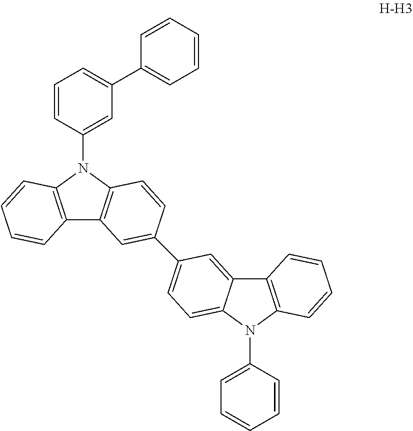 [SMILES: c1ccc(-c2cccc(-n3c4ccccc4c4cc(-c5ccc6c(c5)c5ccccc5n6-c5ccccc5)ccc43)c2)cc1]

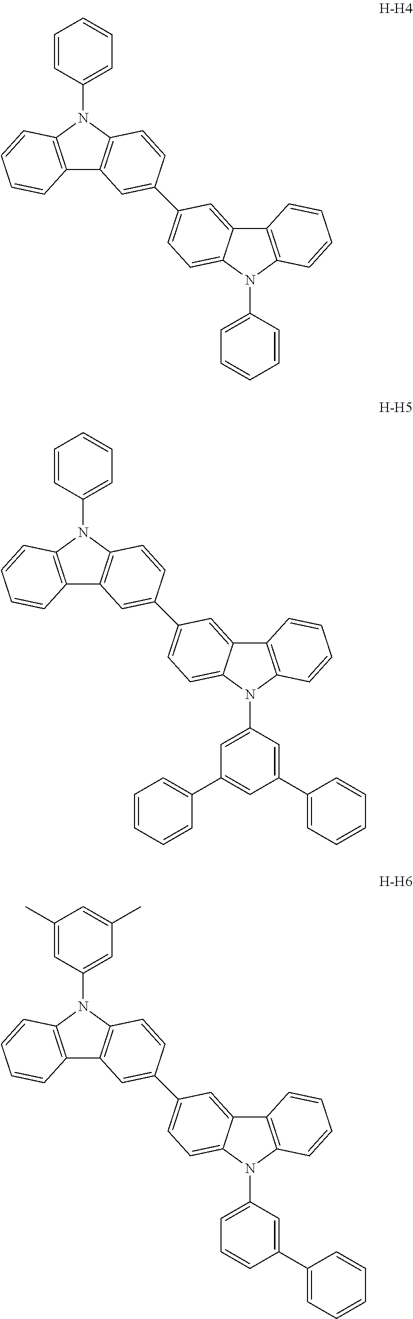 [SMILES: c1ccc(-n2c3ccccc3c3cc(-c4ccc5c(c4)c4ccccc4n5-c4ccccc4)ccc32)cc1 ; c1ccc(-c2cc(-c3ccccc3)cc(-n3c4ccccc4c4cc(-c5ccc6c(c5)c5ccccc5n6-c5ccccc5)ccc43)c2)cc1 ; Cc1cc(C)cc(-n2c3ccccc3c3cc(-c4ccc5c(c4)c4ccccc4n5-c4cccc(-c5ccccc5)c4)ccc32)c1]

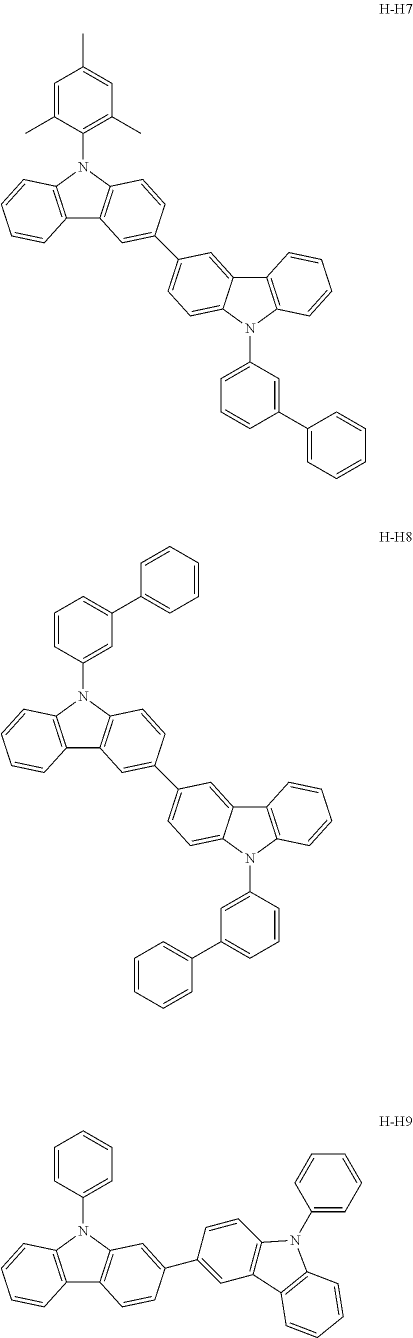 [SMILES: Cc1cc(C)c(-n2c3ccccc3c3cc(-c4ccc5c(c4)c4ccccc4n5-c4cccc(-c5ccccc5)c4)ccc32)c(C)c1 ; c1ccc(-c2cccc(-n3c4ccccc4c4cc(-c5ccc6c(c5)c5ccccc5n6-c5cccc(-c6ccccc6)c5)ccc43)c2)cc1 ; c1ccc(-n2c3ccccc3c3cc(-c4ccc5c6ccccc6n(-c6ccccc6)c5c4)ccc32)cc1]

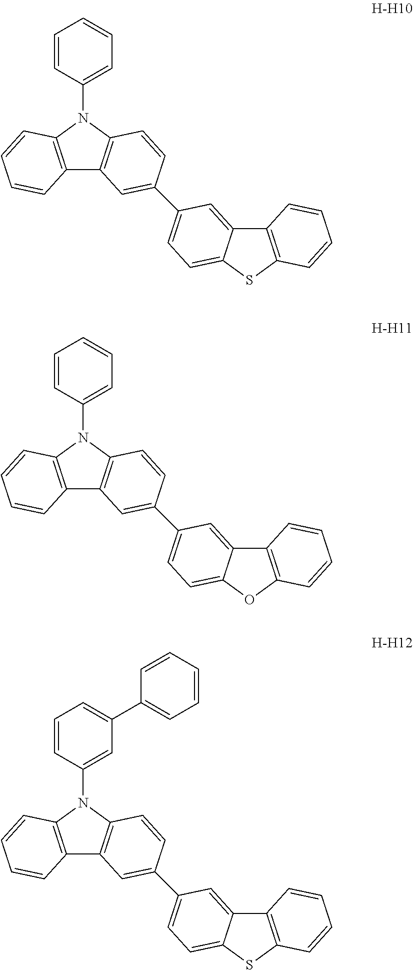 [SMILES: c1ccc(-n2c3ccccc3c3cc(-c4ccc5sc6ccccc6c5c4)ccc32)cc1 ; c1ccc(-n2c3ccccc3c3cc(-c4ccc5oc6ccccc6c5c4)ccc32)cc1 ; c1ccc(-c2cccc(-n3c4ccccc4c4cc(-c5ccc6sc7ccccc7c6c5)ccc43)c2)cc1]

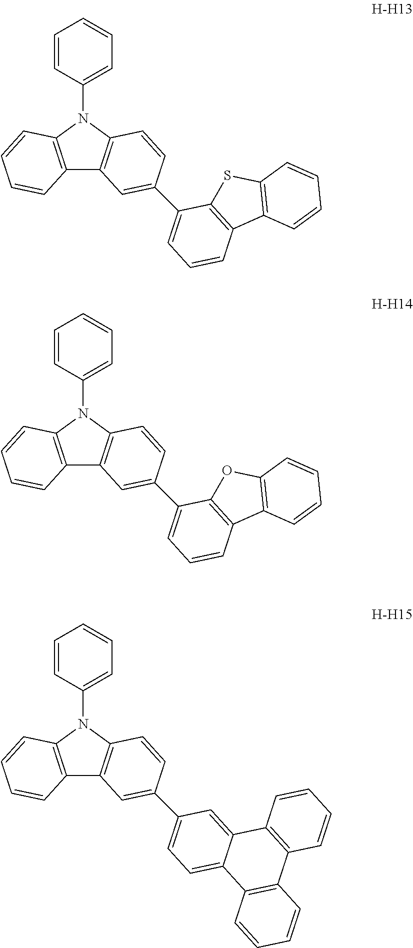 [SMILES: c1ccc(-n2c3ccccc3c3cc(-c4cccc5c4sc4ccccc45)ccc32)cc1 ; c1ccc(-n2c3ccccc3c3cc(-c4cccc5c4oc4ccccc45)ccc32)cc1 ; c1ccc(-n2c3ccccc3c3cc(-c4ccc5c6ccccc6c6ccccc6c5c4)ccc32)cc1]

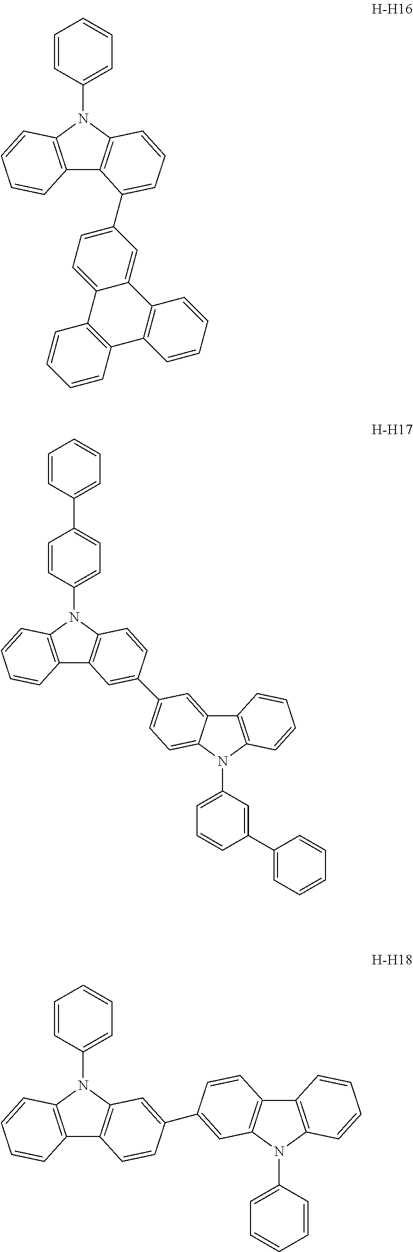 [SMILES: c1ccc(-n2c3ccccc3c3c(-c4ccc5c6ccccc6c6ccccc6c5c4)cccc32)cc1 ; c1ccc(-c2ccc(-n3c4ccccc4c4cc(-c5ccc6c(c5)c5ccccc5n6-c5cccc(-c6ccccc6)c5)ccc43)cc2)cc1 ; c1ccc(-n2c3ccccc3c3ccc(-c4ccc5c6ccccc6n(-c6ccccc6)c5c4)cc32)cc1]

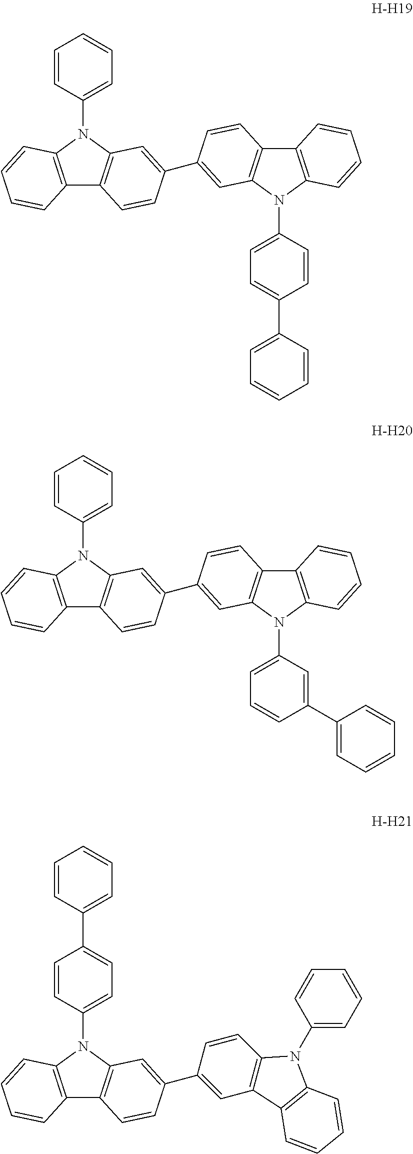

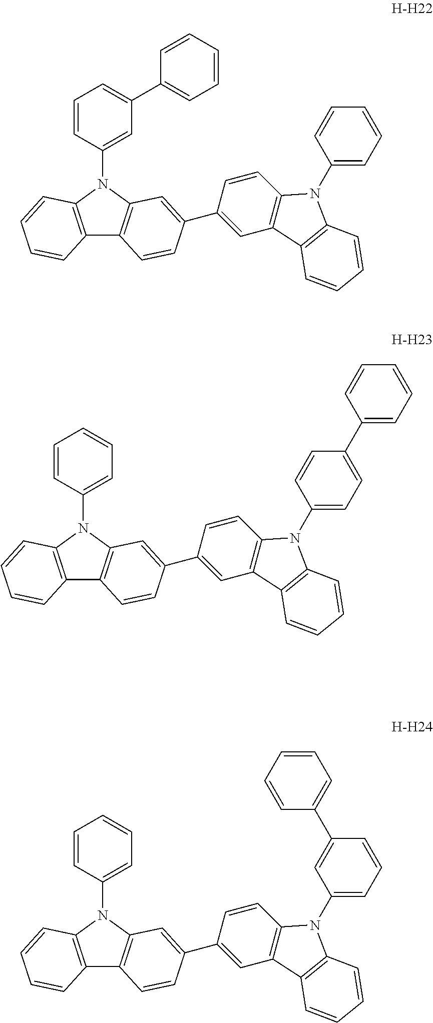 [SMILES: c1ccc(-c2cccc(-n3c4ccccc4c4ccc(-c5ccc6c(c5)c5ccccc5n6-c5ccccc5)cc43)c2)cc1 ; c1ccc(-c2ccc(-n3c4ccccc4c4cc(-c5ccc6c7ccccc7n(-c7ccccc7)c6c5)ccc43)cc2)cc1 ; c1ccc(-c2cccc(-n3c4ccccc4c4cc(-c5ccc6c7ccccc7n(-c7ccccc7)c6c5)ccc43)c2)cc1]

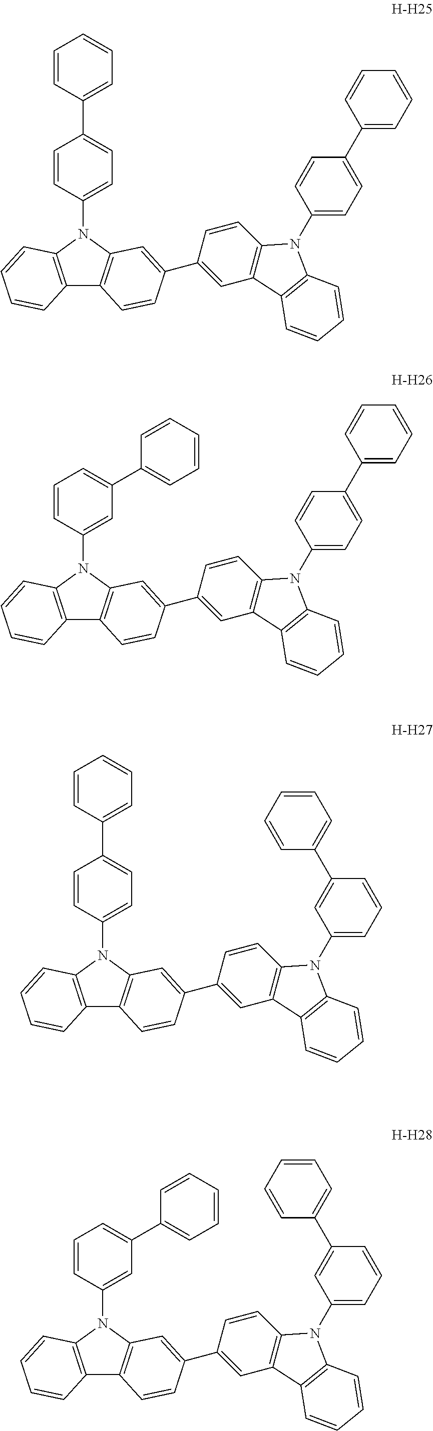 [SMILES: c1ccc(-c2ccc(-n3c4ccccc4c4cc(-c5ccc6c7ccccc7n(-c7ccc(-c8ccccc8)cc7)c6c5)ccc43)cc2)cc1 ; c1ccc(-c2ccc(-n3c4ccccc4c4cc(-c5ccc6c7ccccc7n(-c7cccc(-c8ccccc8)c7)c6c5)ccc43)cc2)cc1 ; c1ccc(-c2ccc(-n3c4ccccc4c4ccc(-c5ccc6c(c5)c5ccccc5n6-c5cccc(-c6ccccc6)c5)cc43)cc2)cc1 ; c1ccc(-c2cccc(-n3c4ccccc4c4cc(-c5ccc6c7ccccc7n(-c7cccc(-c8ccccc8)c7)c6c5)ccc43)c2)cc1]

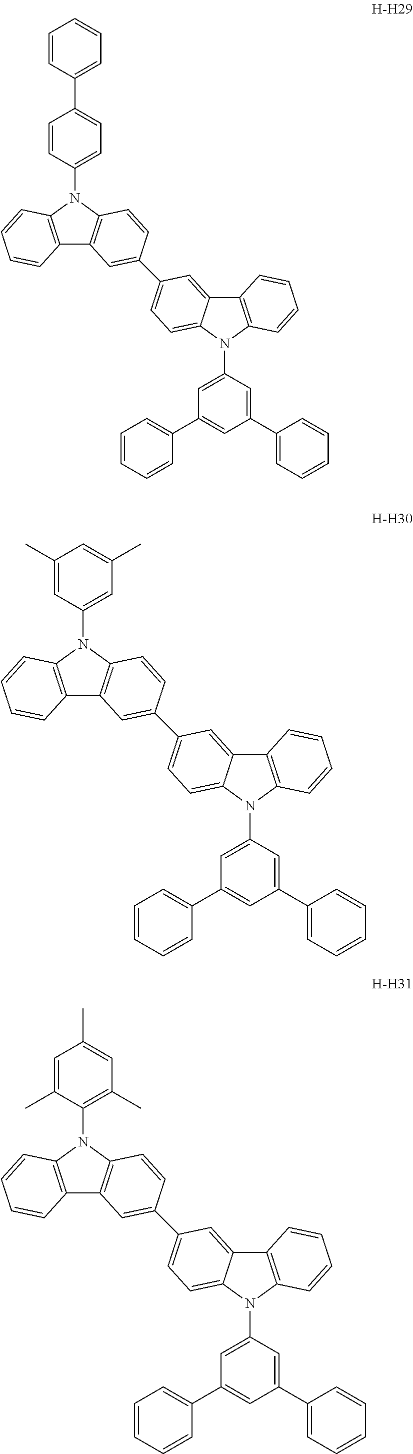 [SMILES: c1ccc(-c2ccc(-n3c4ccccc4c4cc(-c5ccc6c(c5)c5ccccc5n6-c5cc(-c6ccccc6)cc(-c6ccccc6)c5)ccc43)cc2)cc1 ; Cc1cc(C)cc(-n2c3ccccc3c3cc(-c4ccc5c(c4)c4ccccc4n5-c4cc(-c5ccccc5)cc(-c5ccccc5)c4)ccc32)c1 ; Cc1cc(C)c(-n2c3ccccc3c3cc(-c4ccc5c(c4)c4ccccc4n5-c4cc(-c5ccccc5)cc(-c5ccccc5)c4)ccc32)c(C)c1]

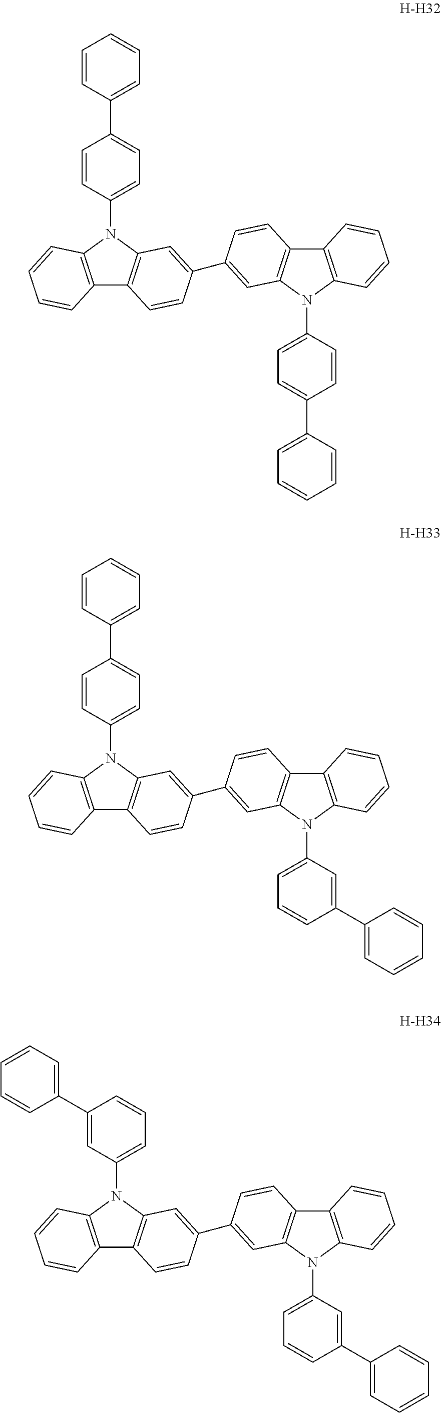 [SMILES: c1ccc(-c2ccc(-n3c4ccccc4c4ccc(-c5ccc6c7ccccc7n(-c7ccc(-c8ccccc8)cc7)c6c5)cc43)cc2)cc1 ; c1ccc(-c2ccc(-n3c4ccccc4c4ccc(-c5ccc6c7ccccc7n(-c7cccc(-c8ccccc8)c7)c6c5)cc43)cc2)cc1 ; c1ccc(-c2cccc(-n3c4ccccc4c4ccc(-c5ccc6c7ccccc7n(-c7cccc(-c8ccccc8)c7)c6c5)cc43)c2)cc1]

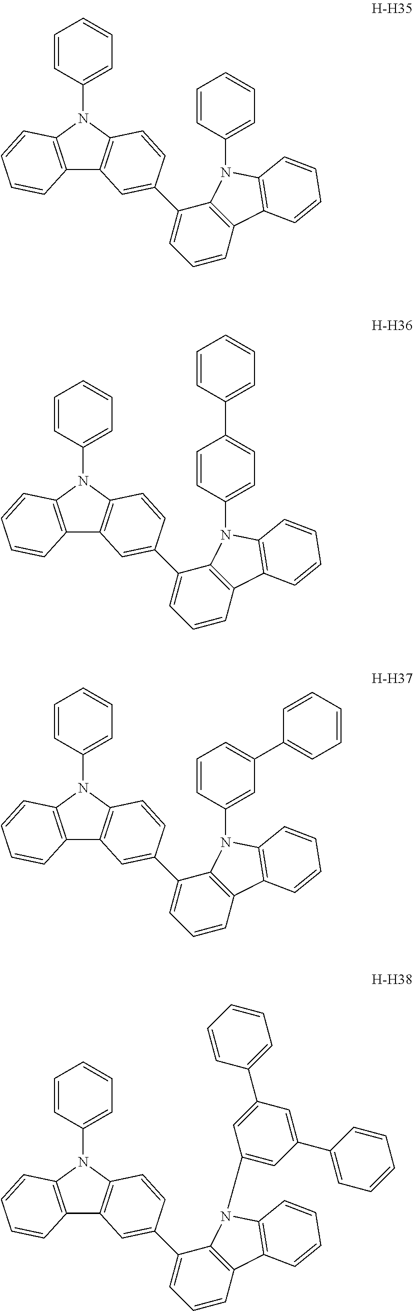 [SMILES: c1ccc(-n2c3ccccc3c3cc(-c4cccc5c6ccccc6n(-c6ccccc6)c45)ccc32)cc1 ; c1ccc(-c2ccc(-n3c4ccccc4c4cccc(-c5ccc6c(c5)c5ccccc5n6-c5ccccc5)c43)cc2)cc1 ; c1ccc(-c2cccc(-n3c4ccccc4c4cccc(-c5ccc6c(c5)c5ccccc5n6-c5ccccc5)c43)c2)cc1 ; c1ccc(-c2cc(-c3ccccc3)cc(-n3c4ccccc4c4cccc(-c5ccc6c(c5)c5ccccc5n6-c5ccccc5)c43)c2)cc1]

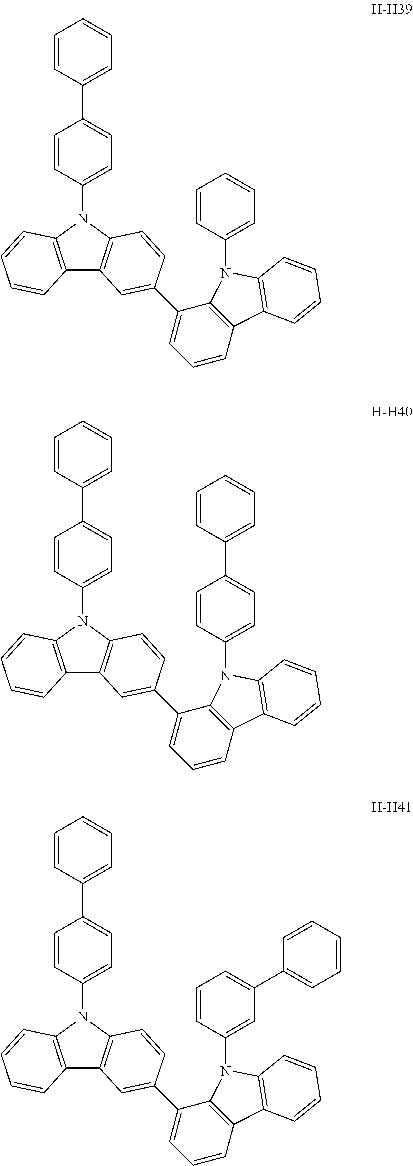 [SMILES: c1ccc(-c2ccc(-n3c4ccccc4c4cc(-c5cccc6c7ccccc7n(-c7ccccc7)c56)ccc43)cc2)cc1 ; c1ccc(-c2ccc(-n3c4ccccc4c4cc(-c5cccc6c7ccccc7n(-c7ccc(-c8ccccc8)cc7)c56)ccc43)cc2)cc1 ; c1ccc(-c2ccc(-n3c4ccccc4c4cc(-c5cccc6c7ccccc7n(-c7cccc(-c8ccccc8)c7)c56)ccc43)cc2)cc1]

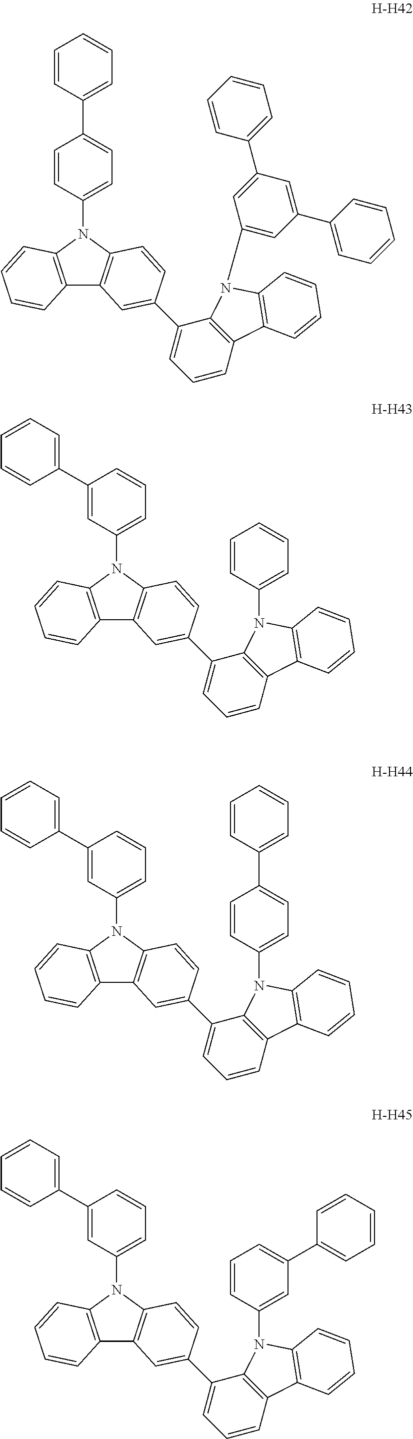 [SMILES: c1ccc(-c2ccc(-n3c4ccccc4c4cc(-c5cccc6c7ccccc7n(-c7cc(-c8ccccc8)cc(-c8ccccc8)c7)c56)ccc43)cc2)cc1 ; c1ccc(-c2cccc(-n3c4ccccc4c4cc(-c5cccc6c7ccccc7n(-c7ccccc7)c56)ccc43)c2)cc1 ; c1ccc(-c2ccc(-n3c4ccccc4c4cccc(-c5ccc6c(c5)c5ccccc5n6-c5cccc(-c6ccccc6)c5)c43)cc2)cc1 ; c1ccc(-c2cccc(-n3c4ccccc4c4cc(-c5cccc6c7ccccc7n(-c7cccc(-c8ccccc8)c7)c56)ccc43)c2)cc1]

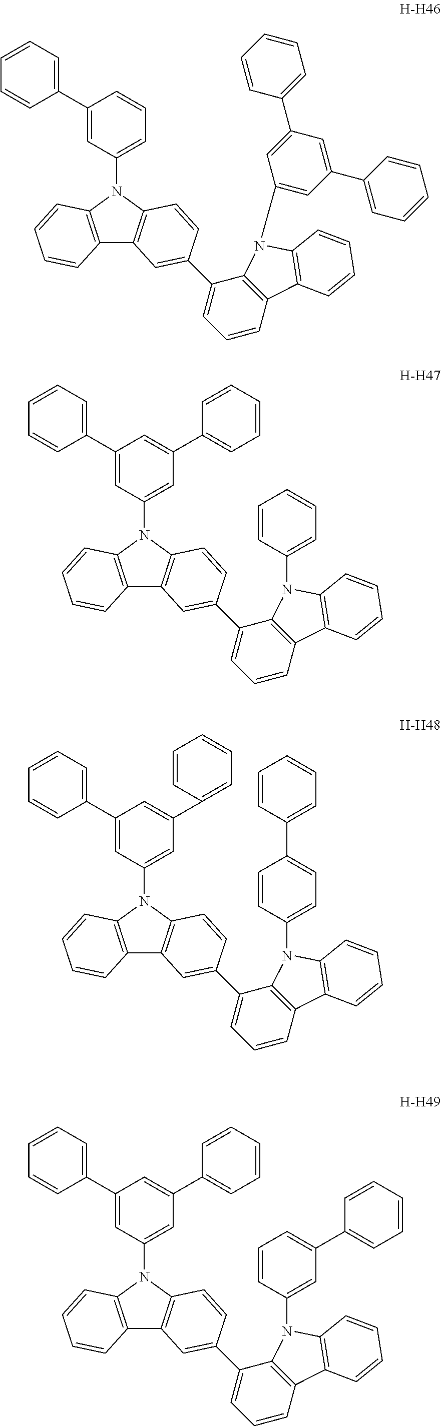

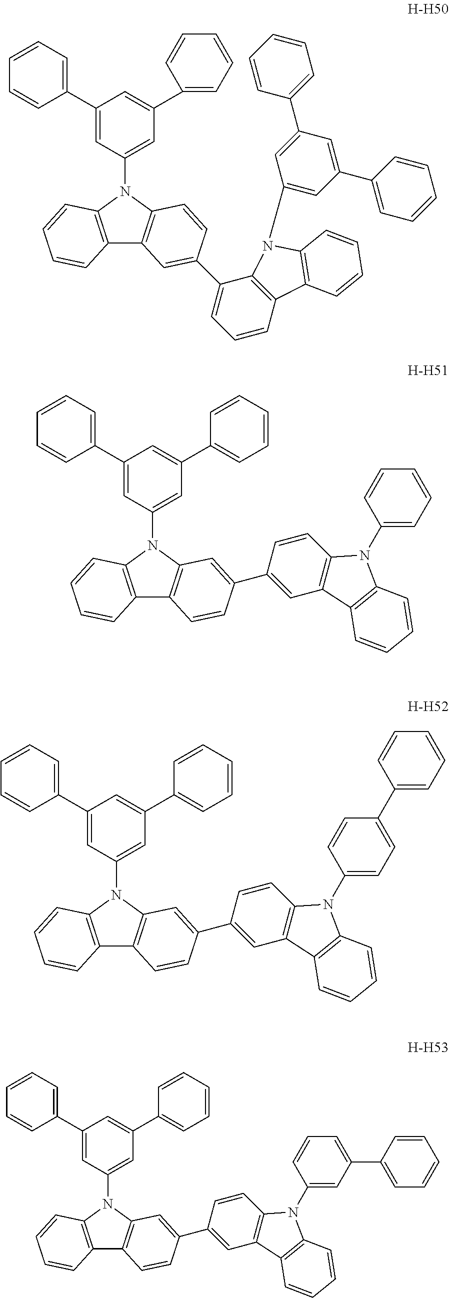 [SMILES: c1ccc(-c2cc(-c3ccccc3)cc(-n3c4ccccc4c4cc(-c5cccc6c7ccccc7n(-c7cc(-c8ccccc8)cc(-c8ccccc8)c7)c56)ccc43)c2)cc1 ; c1ccc(-c2cc(-c3ccccc3)cc(-n3c4ccccc4c4ccc(-c5ccc6c(c5)c5ccccc5n6-c5ccccc5)cc43)c2)cc1 ; c1ccc(-c2ccc(-n3c4ccccc4c4cc(-c5ccc6c7ccccc7n(-c7cc(-c8ccccc8)cc(-c8ccccc8)c7)c6c5)ccc43)cc2)cc1 ; c1ccc(-c2cccc(-n3c4ccccc4c4cc(-c5ccc6c7ccccc7n(-c7cc(-c8ccccc8)cc(-c8ccccc8)c7)c6c5)ccc43)c2)cc1]

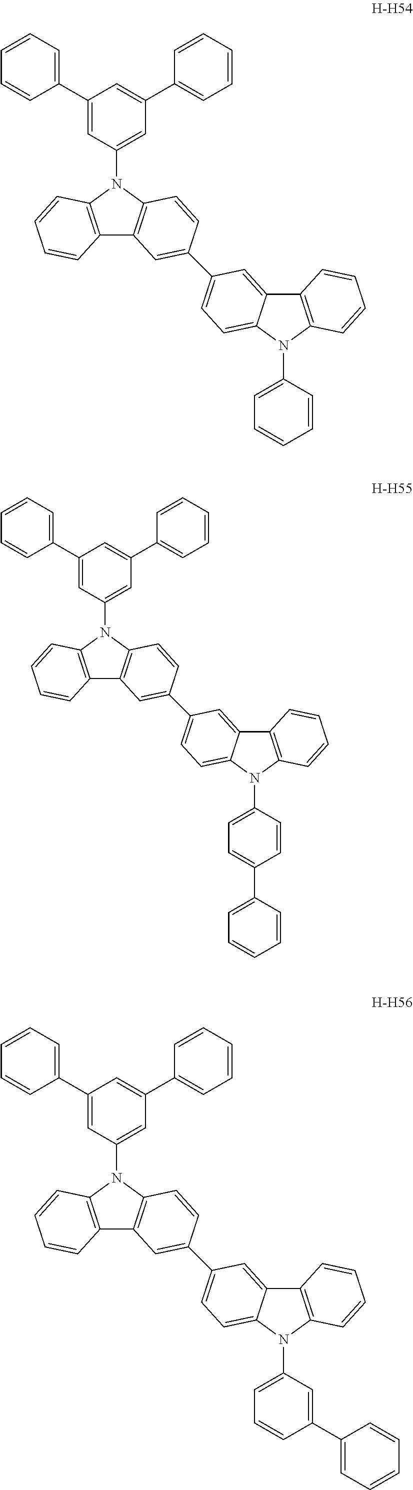 [SMILES: c1ccc(-c2cc(-c3ccccc3)cc(-n3c4ccccc4c4cc(-c5ccc6c(c5)c5ccccc5n6-c5ccccc5)ccc43)c2)cc1 ; c1ccc(-c2ccc(-n3c4ccccc4c4cc(-c5ccc6c(c5)c5ccccc5n6-c5cc(-c6ccccc6)cc(-c6ccccc6)c5)ccc43)cc2)cc1 ; c1ccc(-c2cccc(-n3c4ccccc4c4cc(-c5ccc6c(c5)c5ccccc5n6-c5cc(-c6ccccc6)cc(-c6ccccc6)c5)ccc43)c2)cc1]

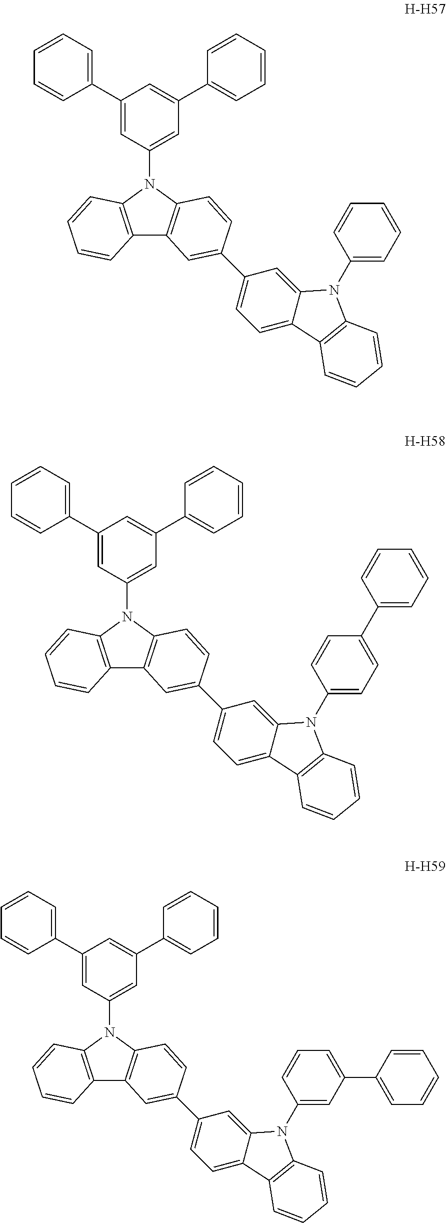 [SMILES: c1ccc(-c2cc(-c3ccccc3)cc(-n3c4ccccc4c4cc(-c5ccc6c7ccccc7n(-c7ccccc7)c6c5)ccc43)c2)cc1 ; c1ccc(-c2ccc(-n3c4ccccc4c4ccc(-c5ccc6c(c5)c5ccccc5n6-c5cc(-c6ccccc6)cc(-c6ccccc6)c5)cc43)cc2)cc1 ; c1ccc(-c2cccc(-n3c4ccccc4c4ccc(-c5ccc6c(c5)c5ccccc5n6-c5cc(-c6ccccc6)cc(-c6ccccc6)c5)cc43)c2)cc1]

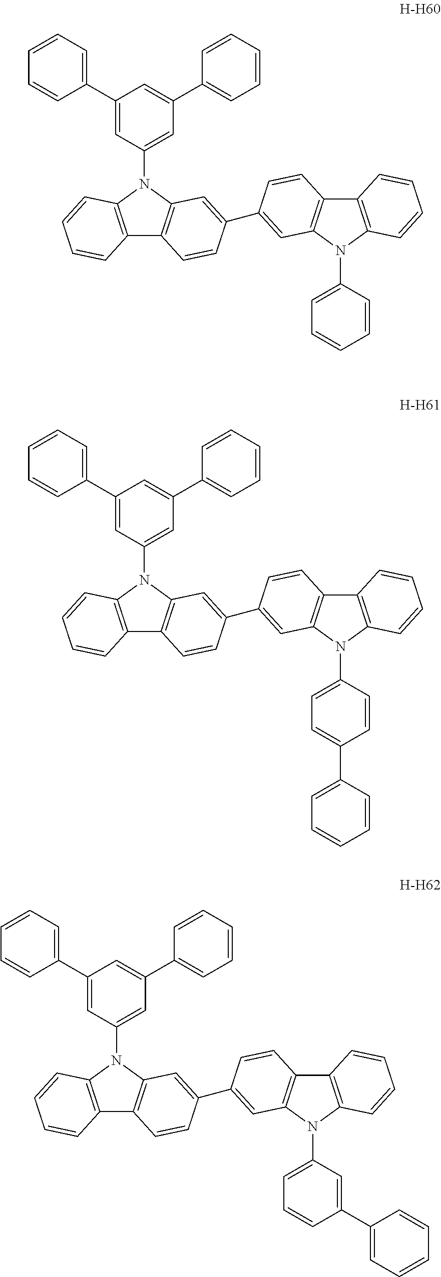 [SMILES: c1ccc(-c2cc(-c3ccccc3)cc(-n3c4ccccc4c4ccc(-c5ccc6c7ccccc7n(-c7ccccc7)c6c5)cc43)c2)cc1 ; c1ccc(-c2ccc(-n3c4ccccc4c4ccc(-c5ccc6c7ccccc7n(-c7cc(-c8ccccc8)cc(-c8ccccc8)c7)c6c5)cc43)cc2)cc1 ; c1ccc(-c2cccc(-n3c4ccccc4c4ccc(-c5ccc6c7ccccc7n(-c7cc(-c8ccccc8)cc(-c8ccccc8)c7)c6c5)cc43)c2)cc1]

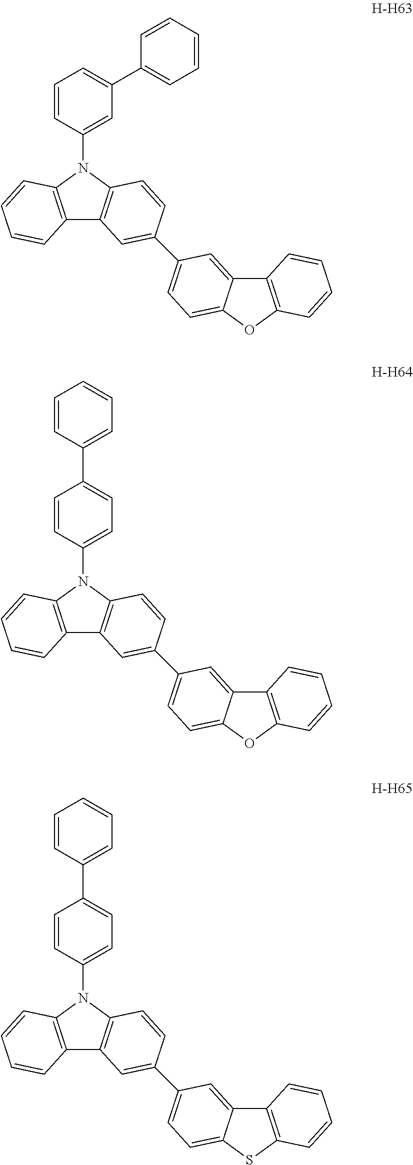 [SMILES: c1ccc(-c2cccc(-n3c4ccccc4c4cc(-c5ccc6oc7ccccc7c6c5)ccc43)c2)cc1 ; c1ccc(-c2ccc(-n3c4ccccc4c4cc(-c5ccc6oc7ccccc7c6c5)ccc43)cc2)cc1 ; c1ccc(-c2ccc(-n3c4ccccc4c4cc(-c5ccc6sc7ccccc7c6c5)ccc43)cc2)cc1]

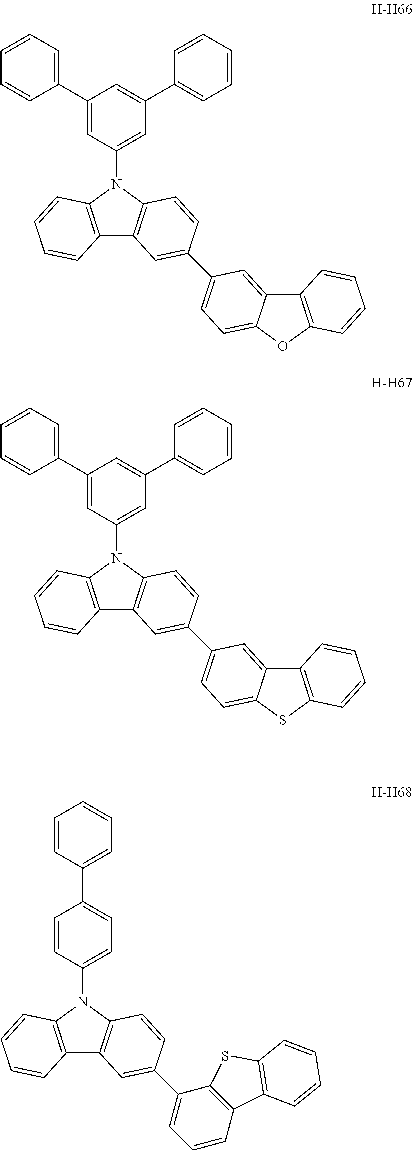 [SMILES: c1ccc(-c2cc(-c3ccccc3)cc(-n3c4ccccc4c4cc(-c5ccc6oc7ccccc7c6c5)ccc43)c2)cc1 ; c1ccc(-c2cc(-c3ccccc3)cc(-n3c4ccccc4c4cc(-c5ccc6sc7ccccc7c6c5)ccc43)c2)cc1 ; c1ccc(-c2ccc(-n3c4ccccc4c4cc(-c5cccc6c5sc5ccccc56)ccc43)cc2)cc1]

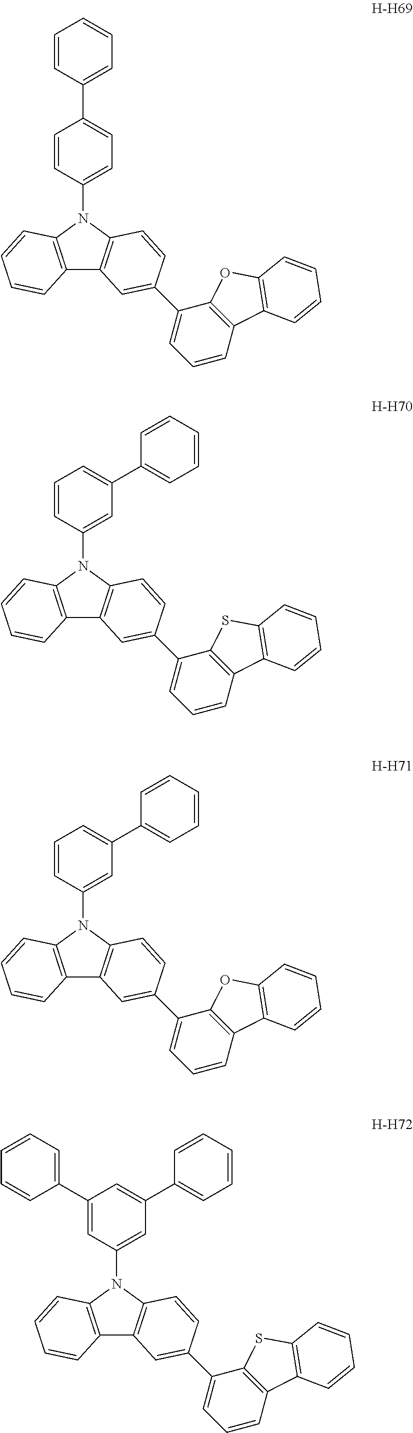 [SMILES: c1ccc(-c2ccc(-n3c4ccccc4c4cc(-c5cccc6c5oc5ccccc56)ccc43)cc2)cc1 ; c1ccc(-c2cccc(-n3c4ccccc4c4cc(-c5cccc6c5sc5ccccc56)ccc43)c2)cc1 ; c1ccc(-c2cccc(-n3c4ccccc4c4cc(-c5cccc6c5oc5ccccc56)ccc43)c2)cc1 ; c1ccc(-c2cc(-c3ccccc3)cc(-n3c4ccccc4c4cc(-c5cccc6c5sc5ccccc56)ccc43)c2)cc1]

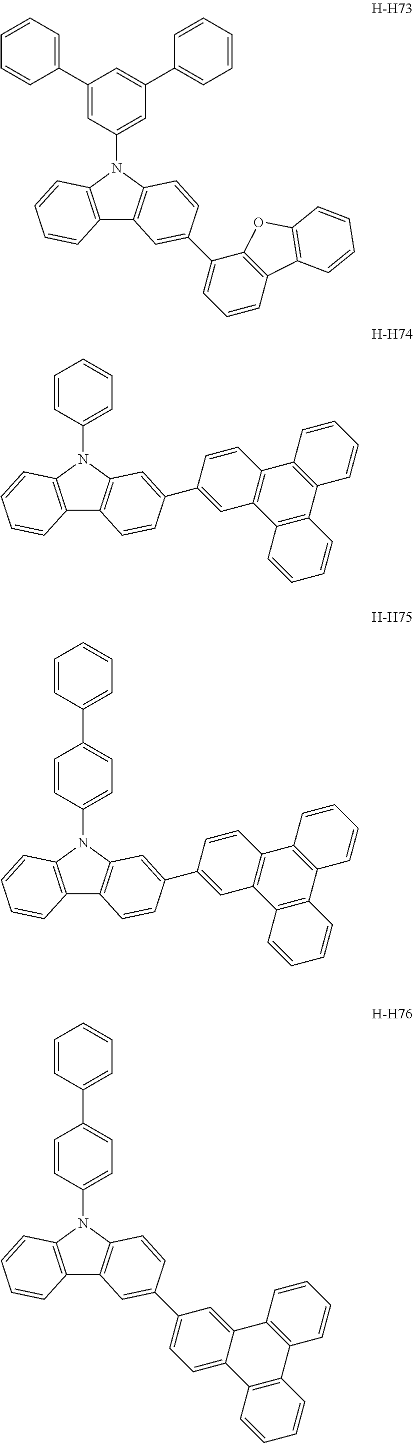 [SMILES: c1ccc(-c2cc(-c3ccccc3)cc(-n3c4ccccc4c4cc(-c5cccc6c5oc5ccccc56)ccc43)c2)cc1 ; c1ccc(-n2c3ccccc3c3ccc(-c4ccc5c6ccccc6c6ccccc6c5c4)cc32)cc1 ; c1ccc(-c2ccc(-n3c4ccccc4c4ccc(-c5ccc6c7ccccc7c7ccccc7c6c5)cc43)cc2)cc1 ; c1ccc(-c2ccc(-n3c4ccccc4c4cc(-c5ccc6c7ccccc7c7ccccc7c6c5)ccc43)cc2)cc1]

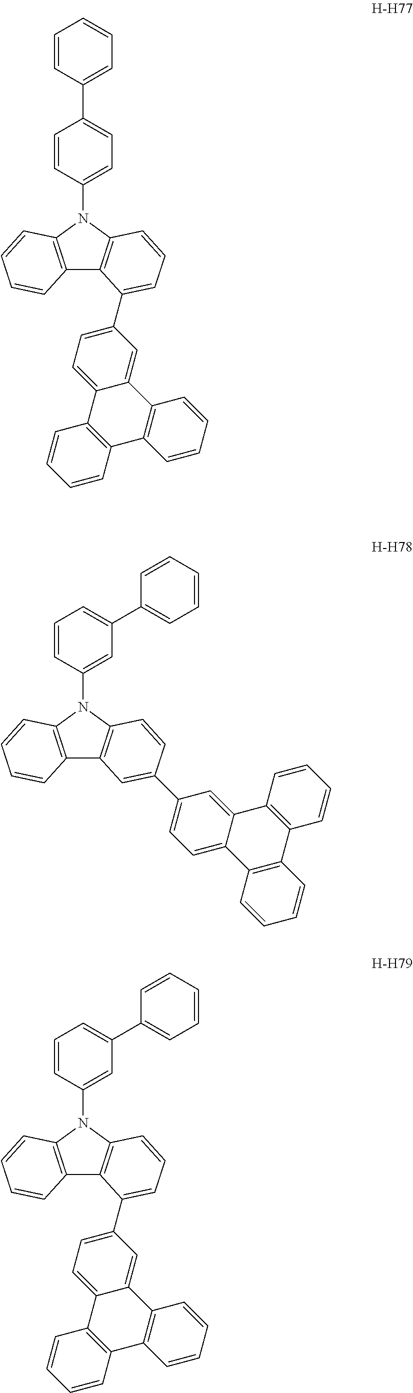 [SMILES: c1ccc(-c2ccc(-n3c4ccccc4c4c(-c5ccc6c7ccccc7c7ccccc7c6c5)cccc43)cc2)cc1 ; c1ccc(-c2cccc(-n3c4ccccc4c4cc(-c5ccc6c7ccccc7c7ccccc7c6c5)ccc43)c2)cc1 ; c1ccc(-c2cccc(-n3c4ccccc4c4c(-c5ccc6c7ccccc7c7ccccc7c6c5)cccc43)c2)cc1]

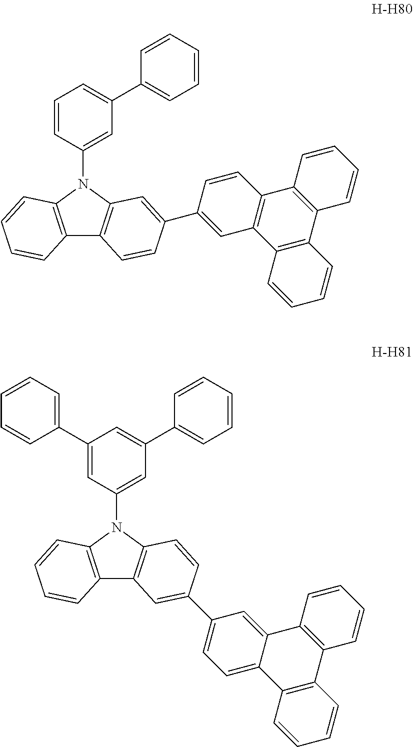 [SMILES: c1ccc(-c2cccc(-n3c4ccccc4c4ccc(-c5ccc6c7ccccc7c7ccccc7c6c5)cc43)c2)cc1 ; c1ccc(-c2cc(-c3ccccc3)cc(-n3c4ccccc4c4cc(-c5ccc6c7ccccc7c7ccccc7c6c5)ccc43)c2)cc1]

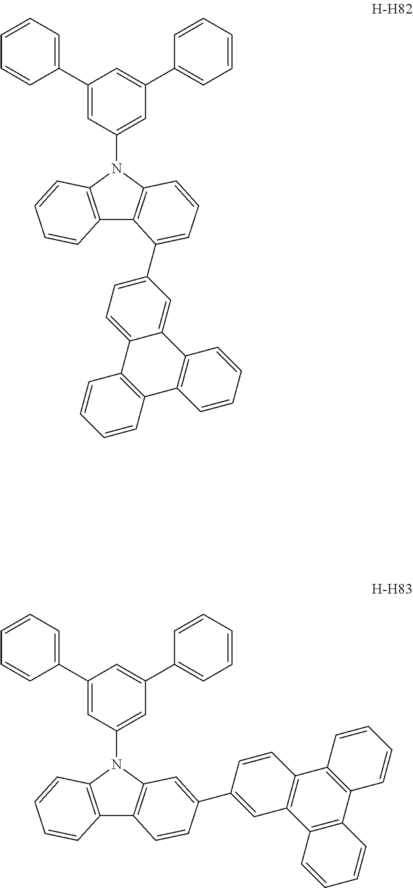 [SMILES: c1ccc(-c2cc(-c3ccccc3)cc(-n3c4ccccc4c4c(-c5ccc6c7ccccc7c7ccccc7c6c5)cccc43)c2)cc1 ; c1ccc(-c2cc(-c3ccccc3)cc(-n3c4ccccc4c4ccc(-c5ccc6c7ccccc7c7ccccc7c6c5)cc43)c2)cc1]

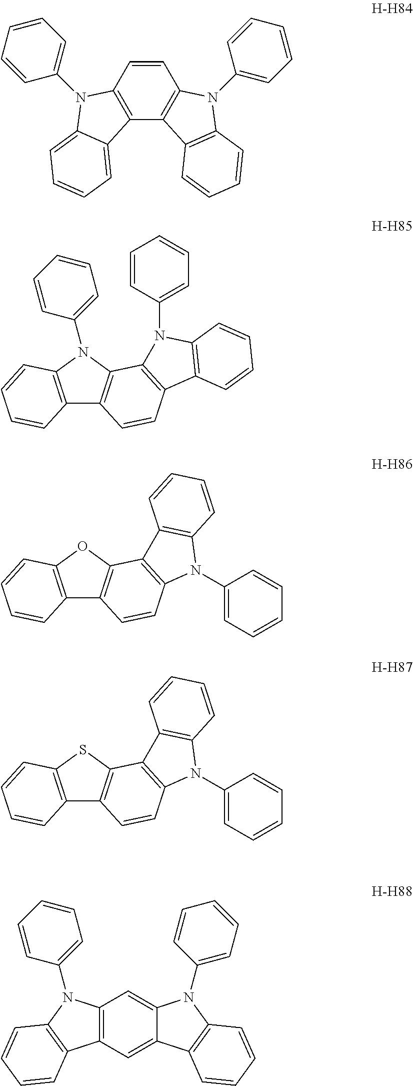 [SMILES: c1ccc(-n2c3ccccc3c3c4c5ccccc5n(-c5ccccc5)c4ccc32)cc1 ; c1ccc(-n2c3ccccc3c3ccc4c5ccccc5n(-c5ccccc5)c4c32)cc1 ; c1ccc(-n2c3ccccc3c3c4oc5ccccc5c4ccc32)cc1 ; c1ccc(-n2c3ccccc3c3c4sc5ccccc5c4ccc32)cc1 ; c1ccc(-n2c3ccccc3c3cc4c5ccccc5n(-c5ccccc5)c4cc32)cc1]

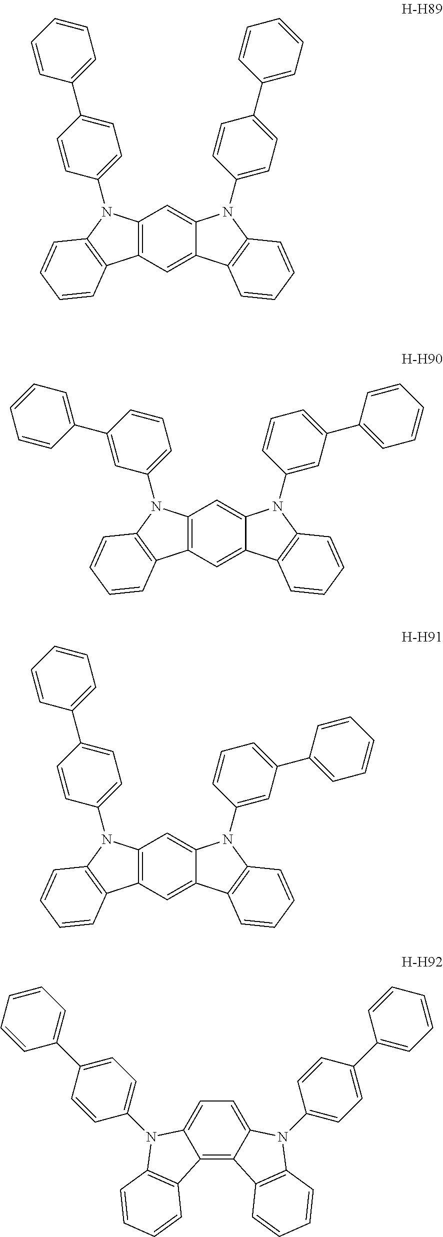 [SMILES: c1ccc(-c2ccc(-n3c4ccccc4c4cc5c6ccccc6n(-c6ccc(-c7ccccc7)cc6)c5cc43)cc2)cc1 ; c1ccc(-c2cccc(-n3c4ccccc4c4cc5c6ccccc6n(-c6cccc(-c7ccccc7)c6)c5cc43)c2)cc1 ; c1ccc(-c2ccc(-n3c4ccccc4c4cc5c6ccccc6n(-c6cccc(-c7ccccc7)c6)c5cc43)cc2)cc1 ; c1ccc(-c2ccc(-n3c4ccccc4c4c5c6ccccc6n(-c6ccc(-c7ccccc7)cc6)c5ccc43)cc2)cc1]

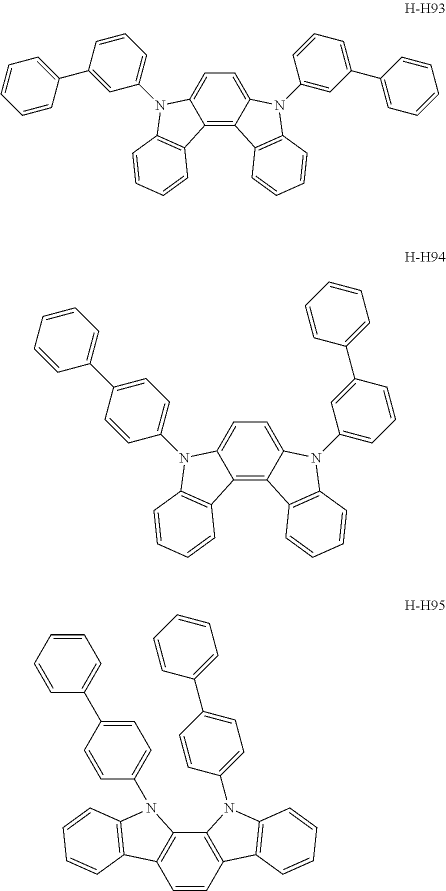 [SMILES: c1ccc(-c2cccc(-n3c4ccccc4c4c5c6ccccc6n(-c6cccc(-c7ccccc7)c6)c5ccc43)c2)cc1 ; c1ccc(-c2ccc(-n3c4ccccc4c4c5c6ccccc6n(-c6cccc(-c7ccccc7)c6)c5ccc43)cc2)cc1 ; c1ccc(-c2ccc(-n3c4ccccc4c4ccc5c6ccccc6n(-c6ccc(-c7ccccc7)cc6)c5c43)cc2)cc1]

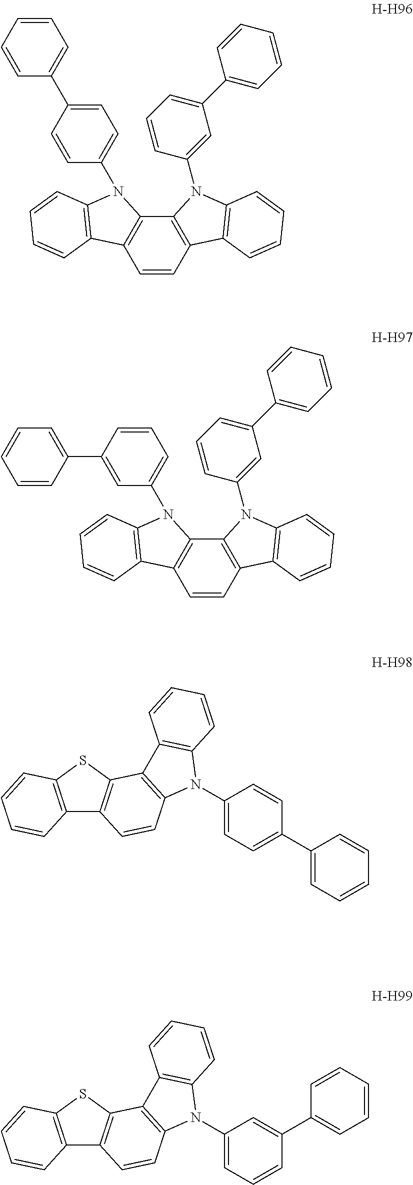 [SMILES: c1ccc(-c2ccc(-n3c4ccccc4c4ccc5c6ccccc6n(-c6cccc(-c7ccccc7)c6)c5c43)cc2)cc1 ; c1ccc(-c2cccc(-n3c4ccccc4c4ccc5c6ccccc6n(-c6cccc(-c7ccccc7)c6)c5c43)c2)cc1 ; c1ccc(-c2ccc(-n3c4ccccc4c4c5sc6ccccc6c5ccc43)cc2)cc1 ; c1ccc(-c2cccc(-n3c4ccccc4c4c5sc6ccccc6c5ccc43)c2)cc1]

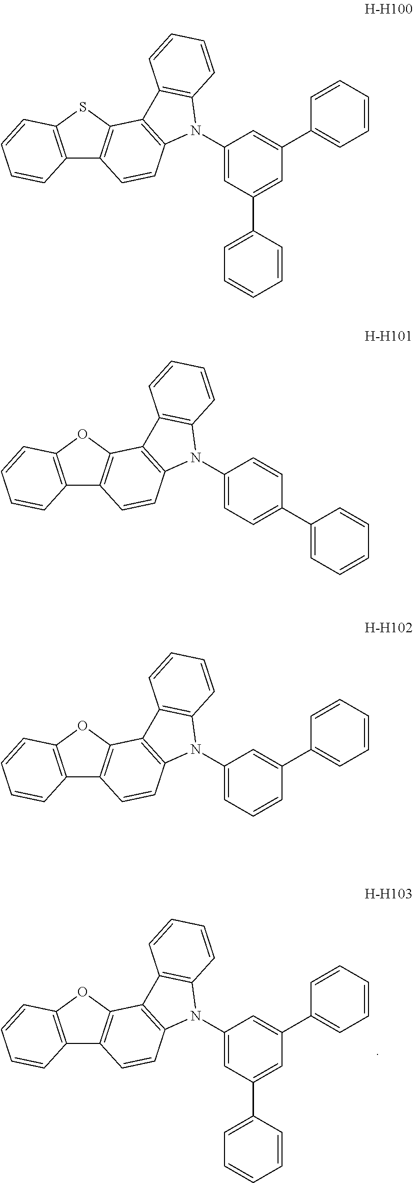 [SMILES: c1ccc(-c2cc(-c3ccccc3)cc(-n3c4ccccc4c4c5sc6ccccc6c5ccc43)c2)cc1 ; c1ccc(-c2ccc(-n3c4ccccc4c4c5oc6ccccc6c5ccc43)cc2)cc1 ; c1ccc(-c2cccc(-n3c4ccccc4c4c5oc6ccccc6c5ccc43)c2)cc1 ; c1ccc(-c2cc(-c3ccccc3)cc(-n3c4ccccc4c4c5oc6ccccc6c5ccc43)c2)cc1]

In one or more embodiments, the bipolar host may be compounds of Group HEH1, but embodiments are not limited thereto:

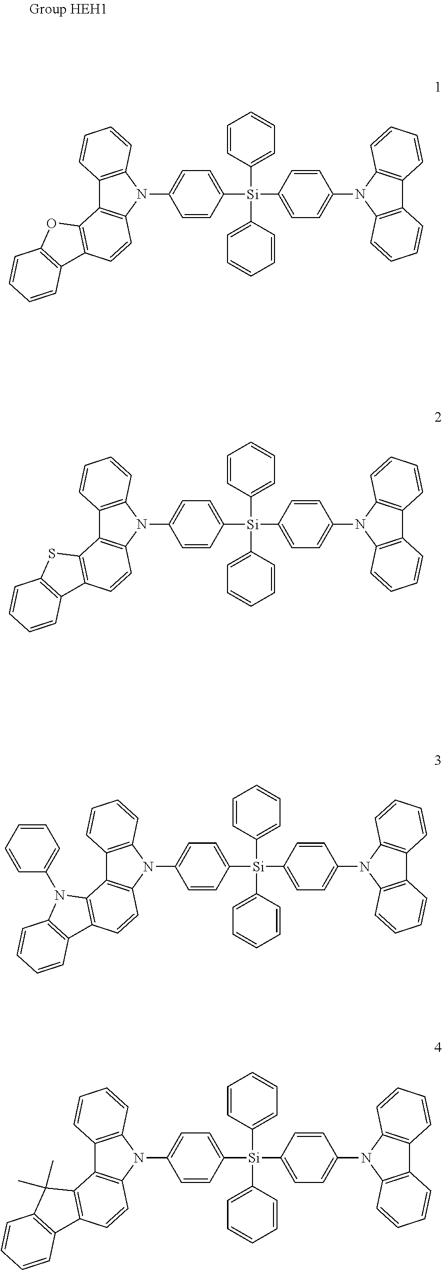

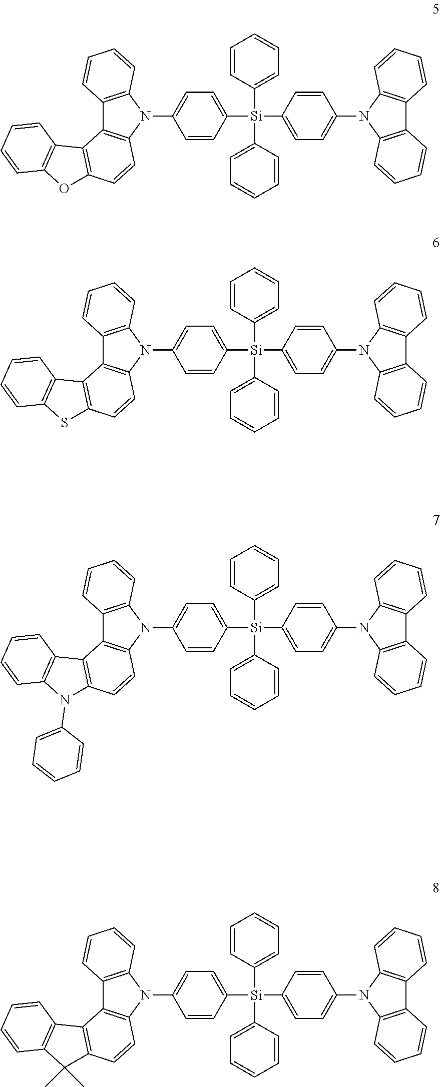

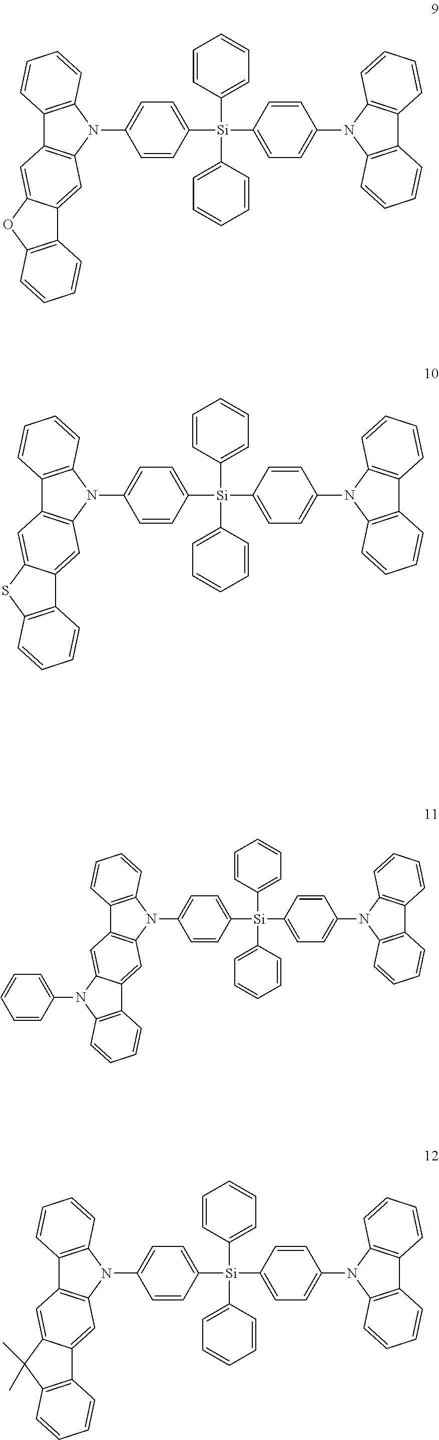

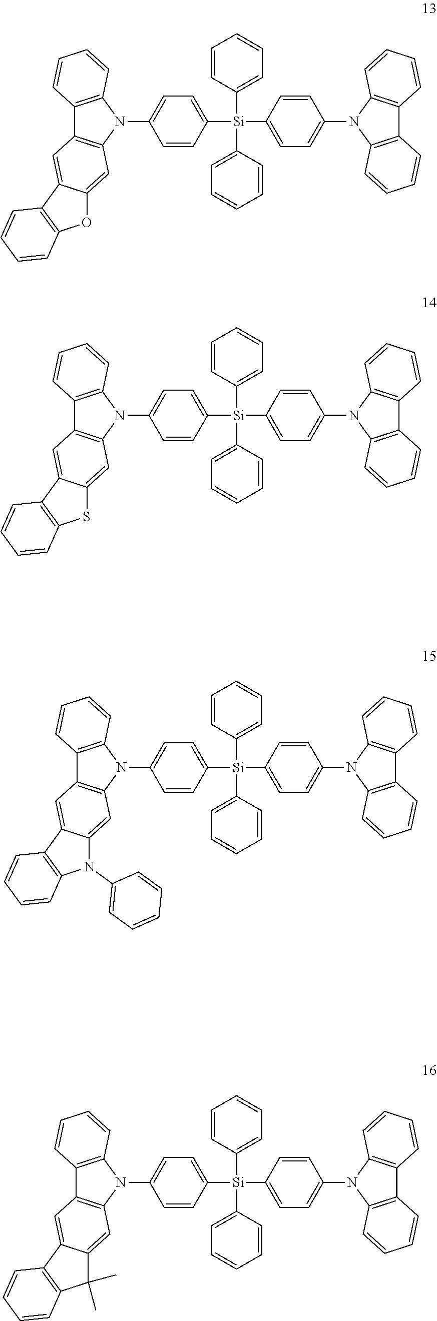

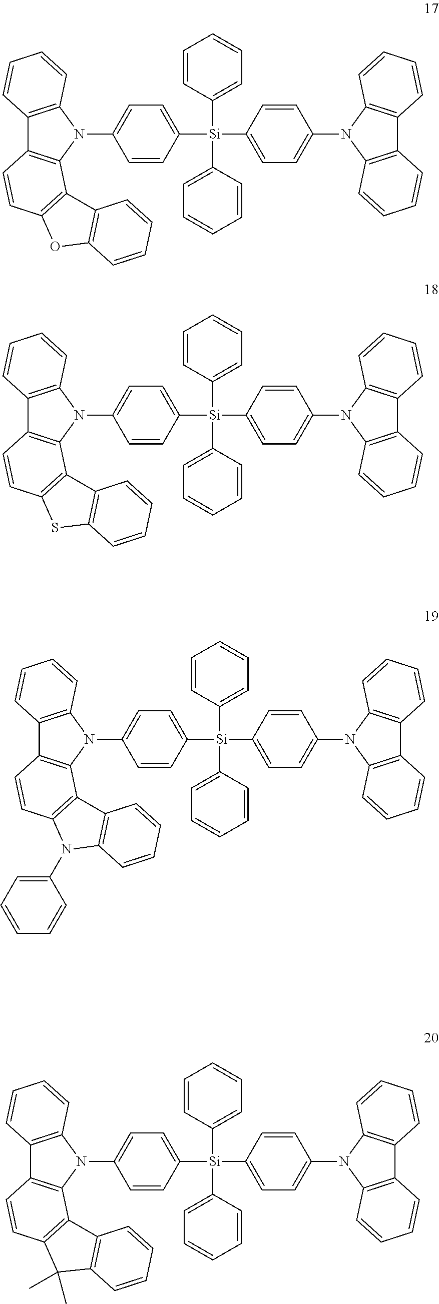

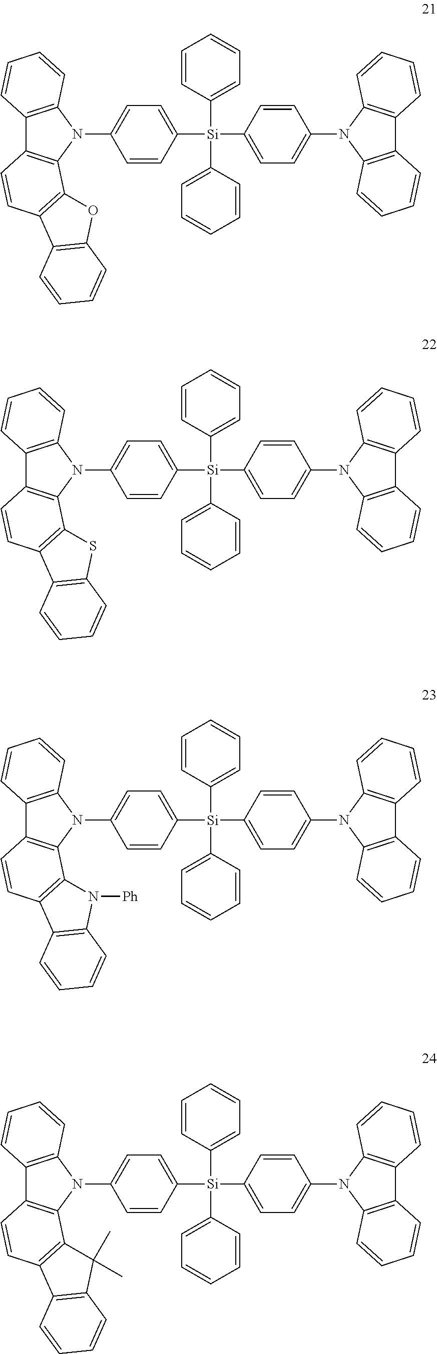

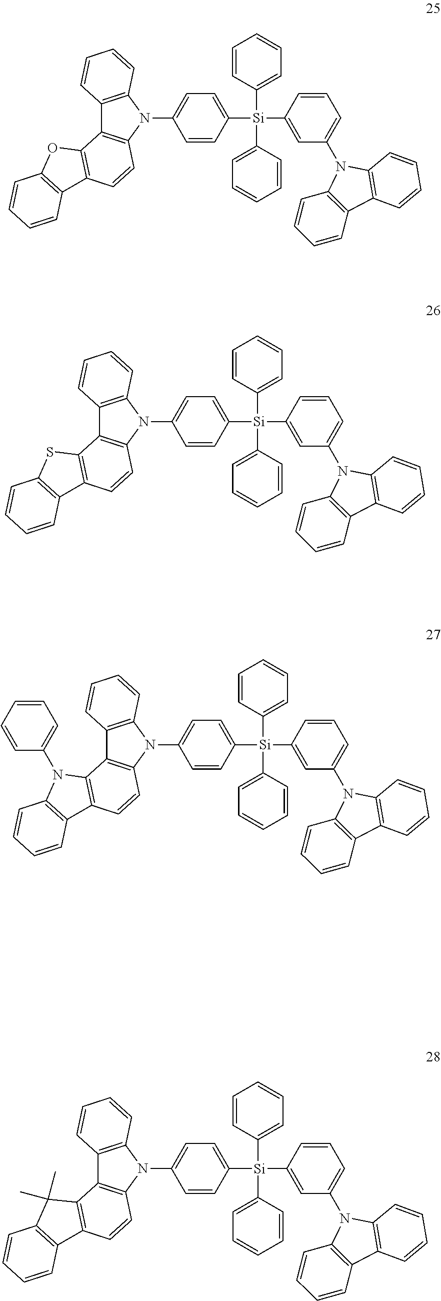

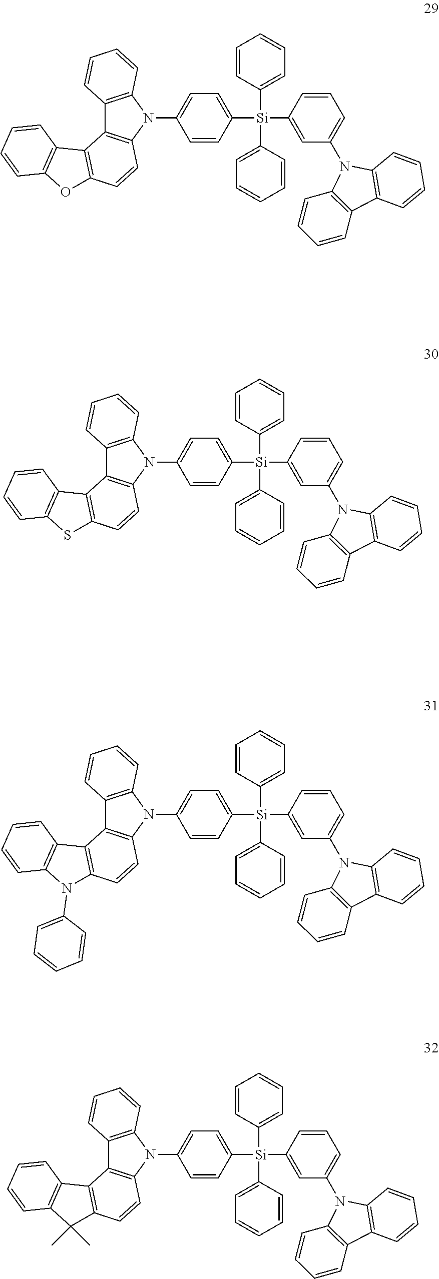

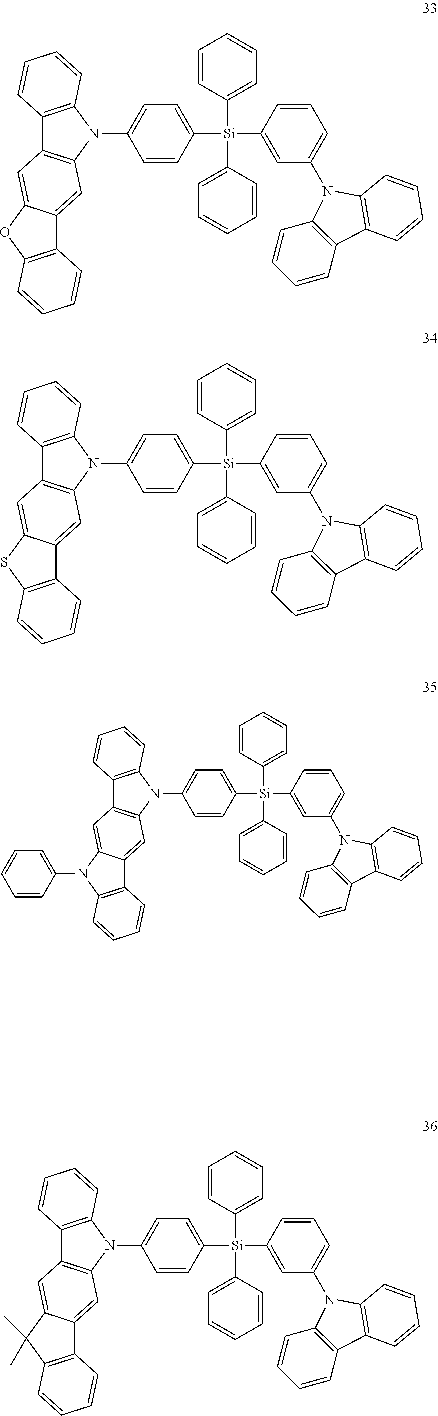

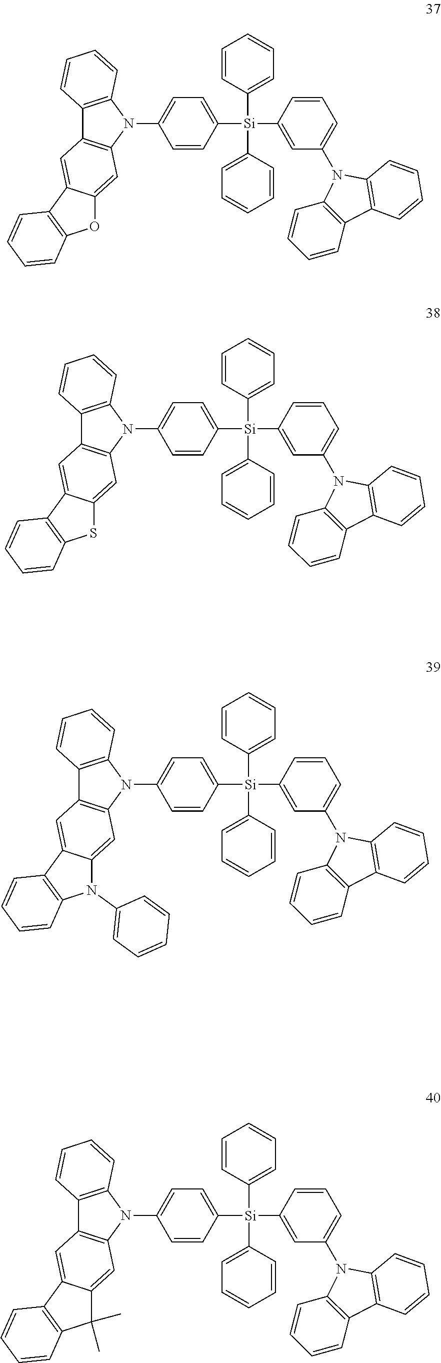

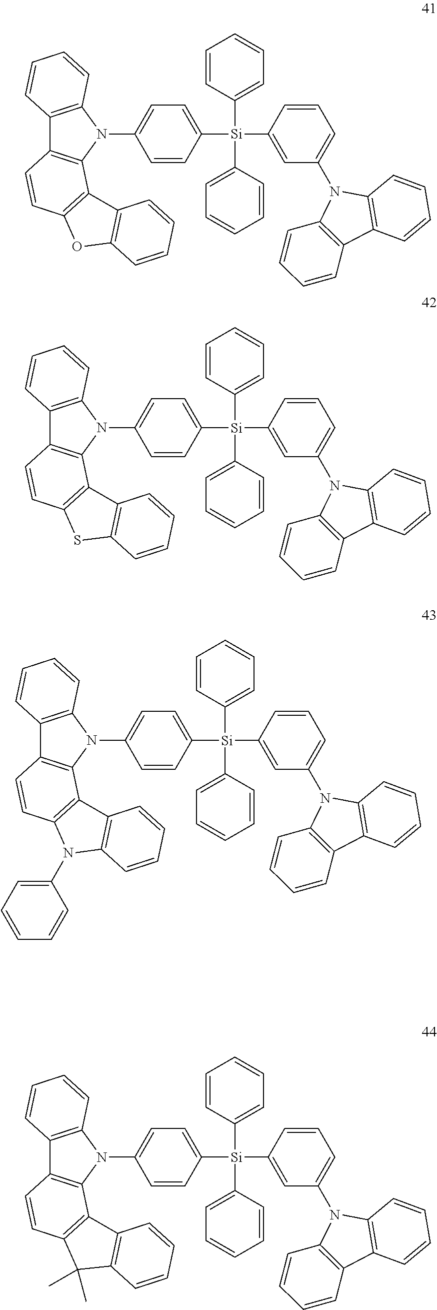

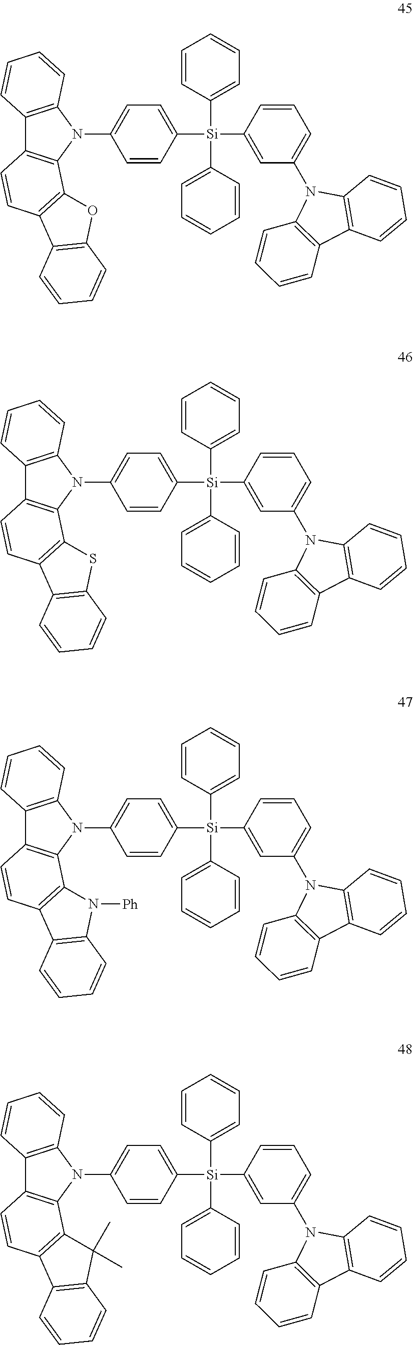

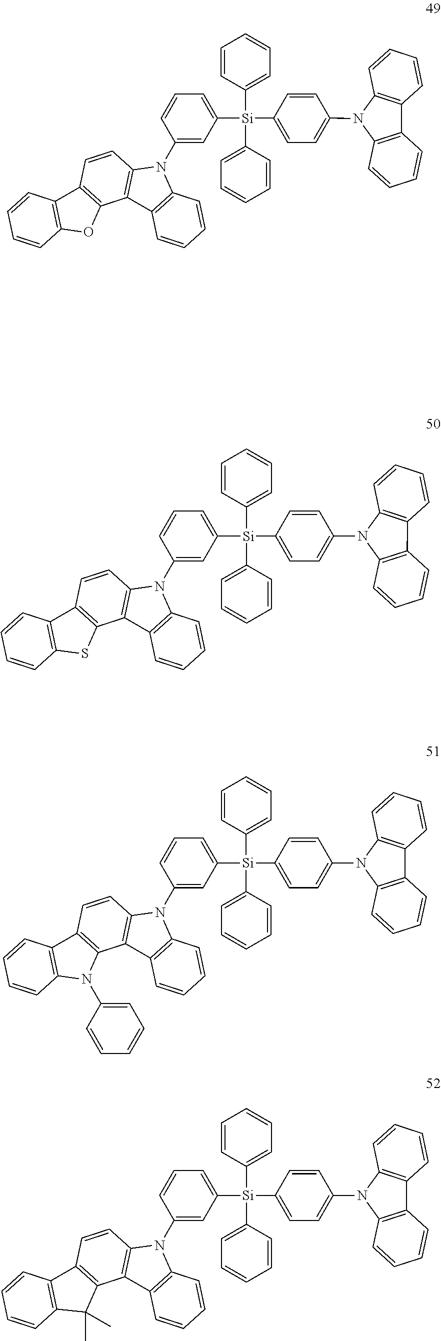

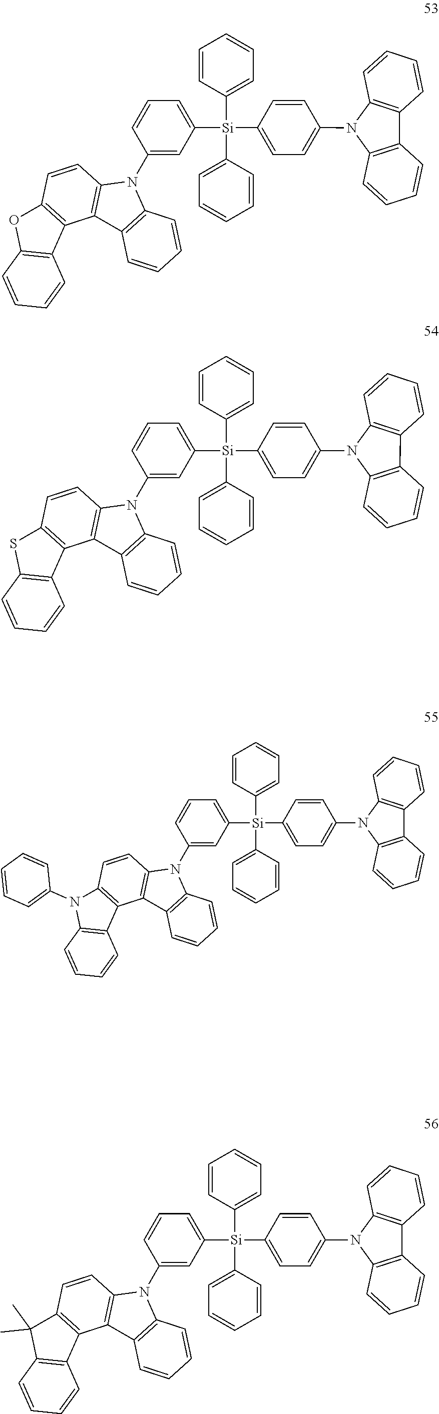

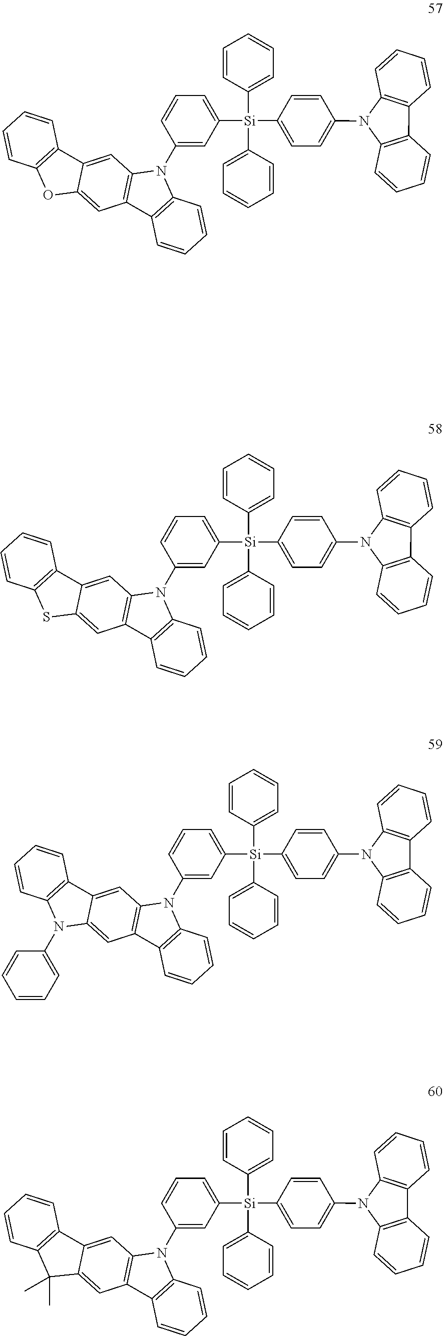

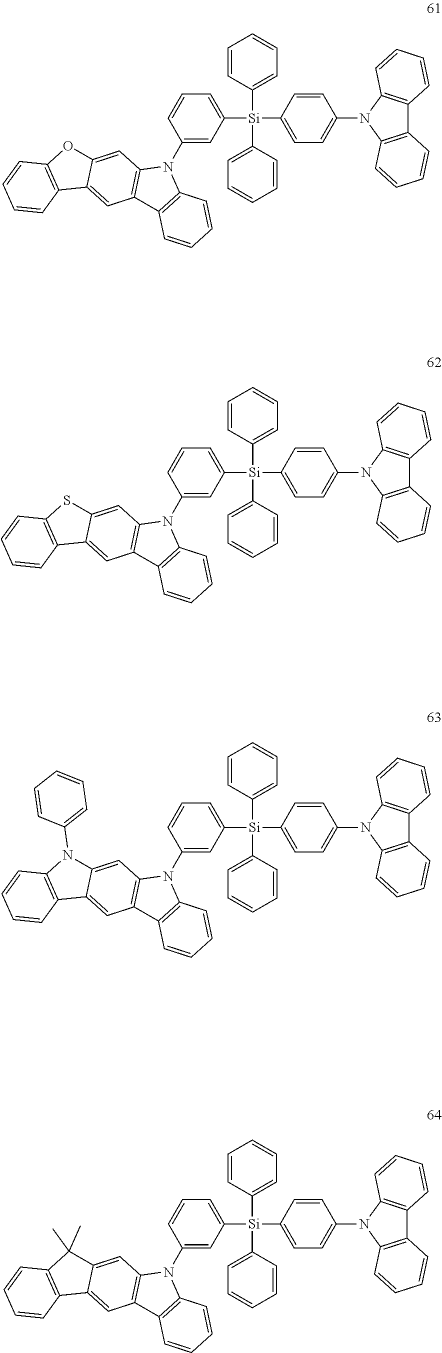

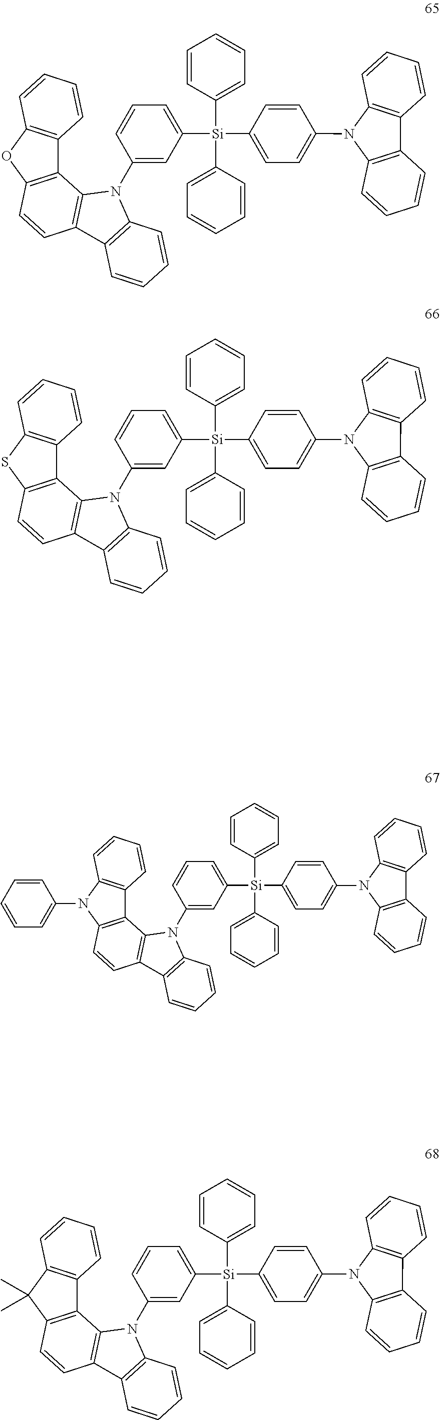

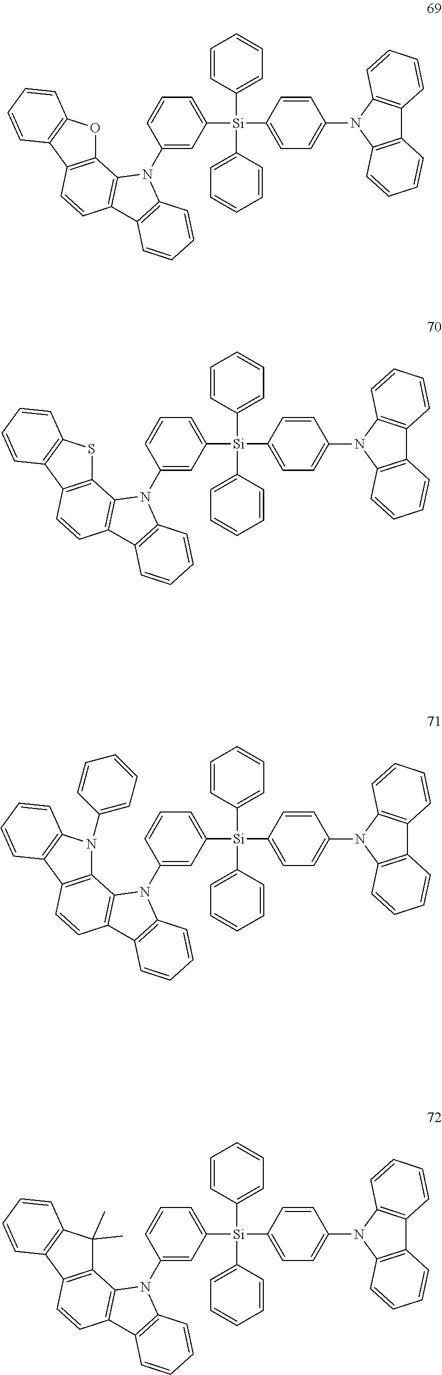

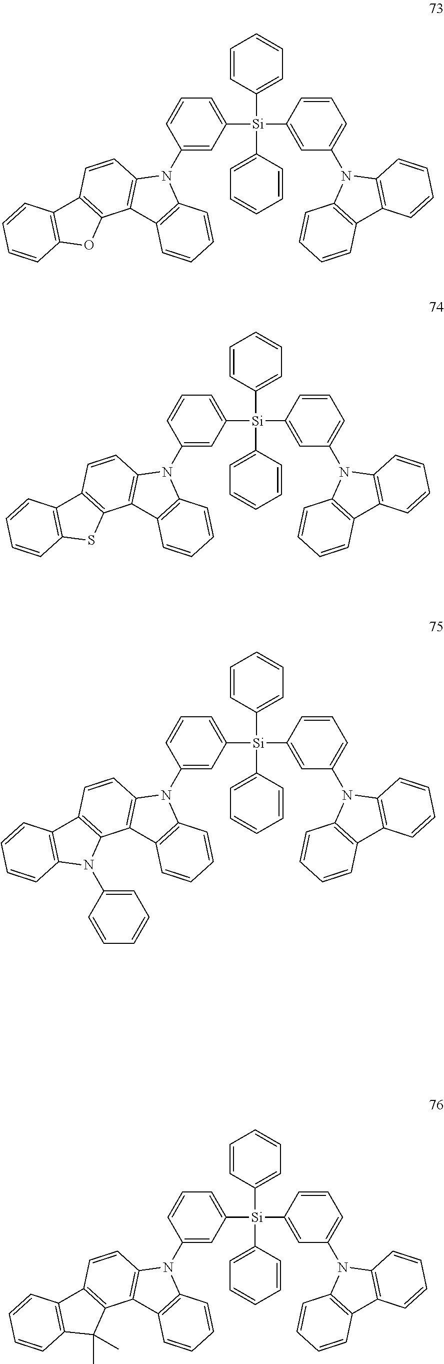

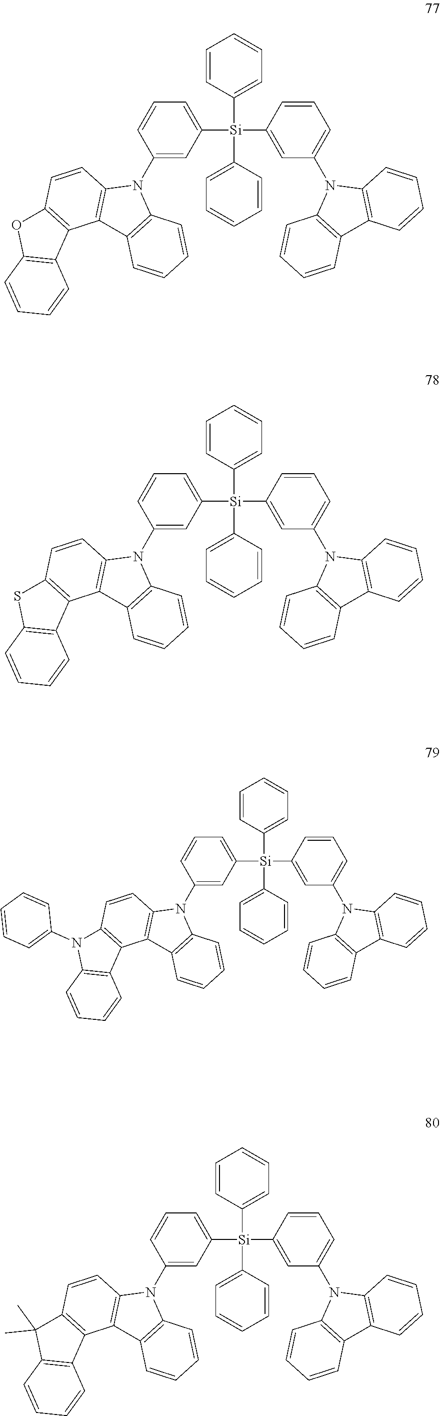

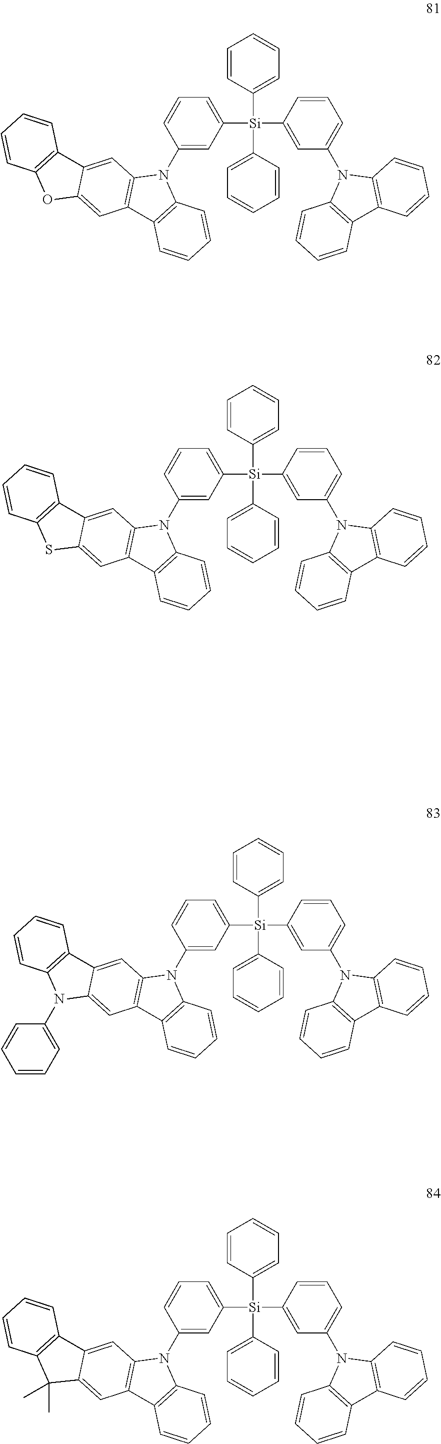

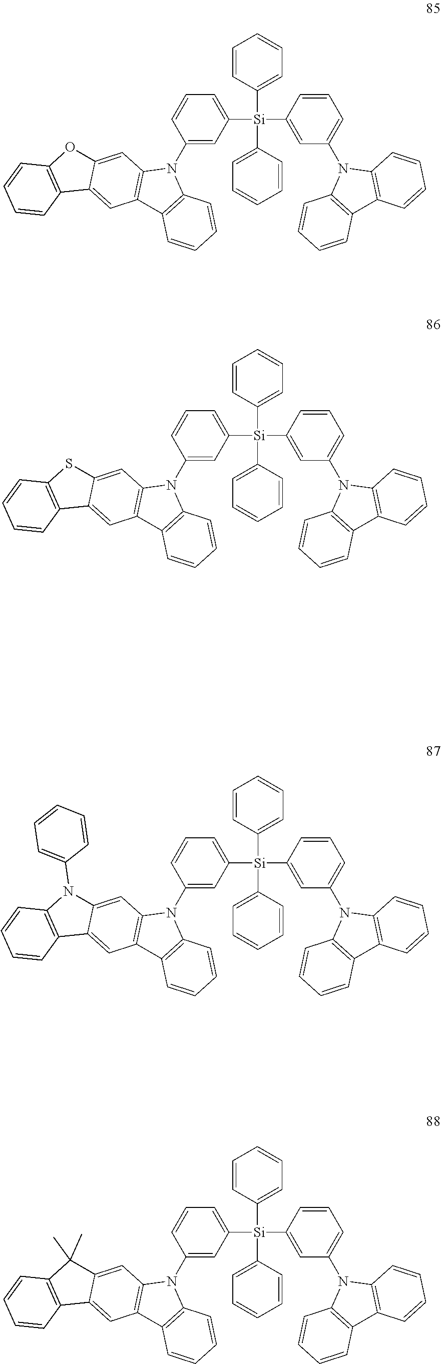

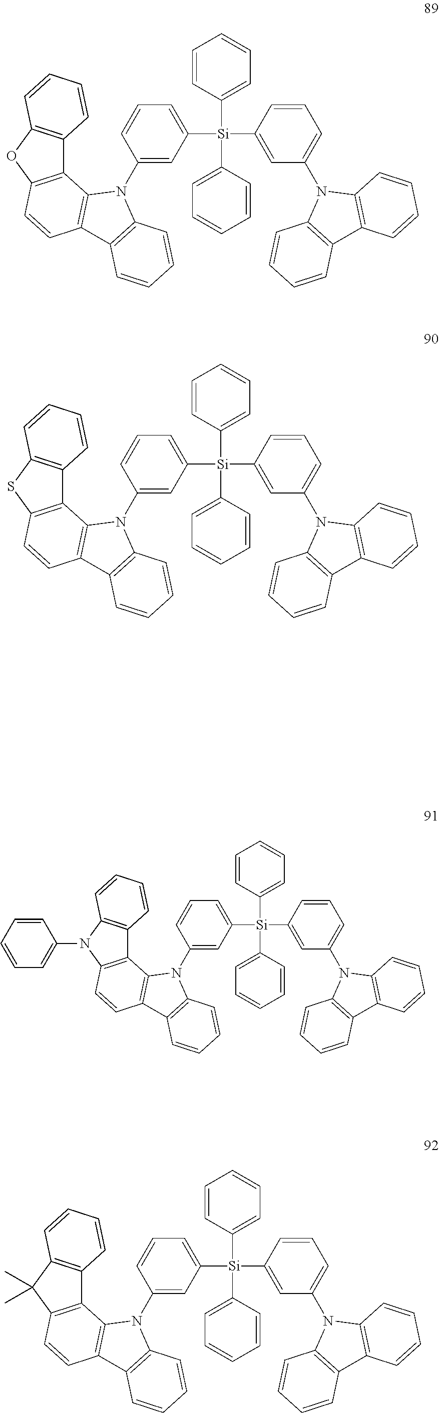

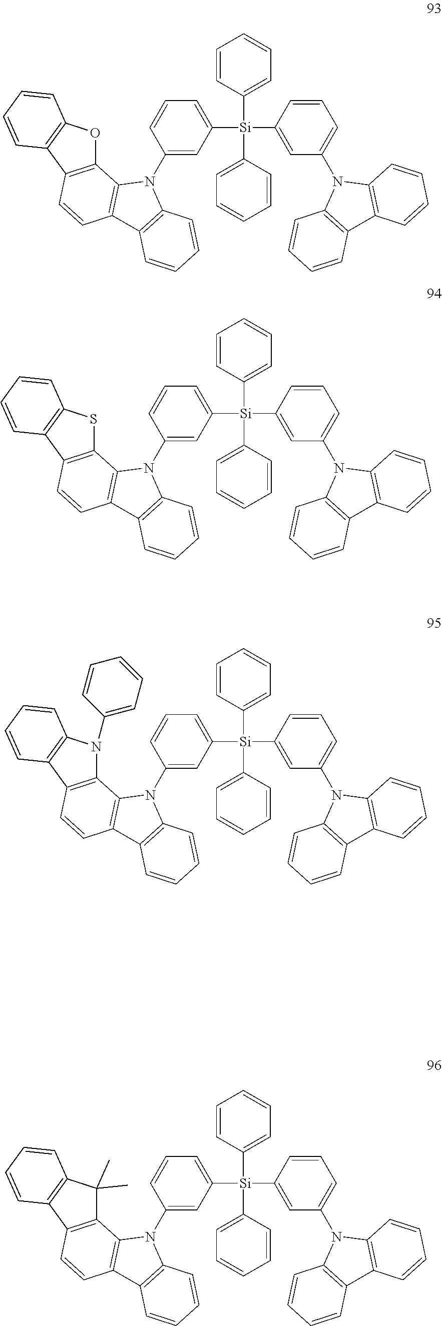

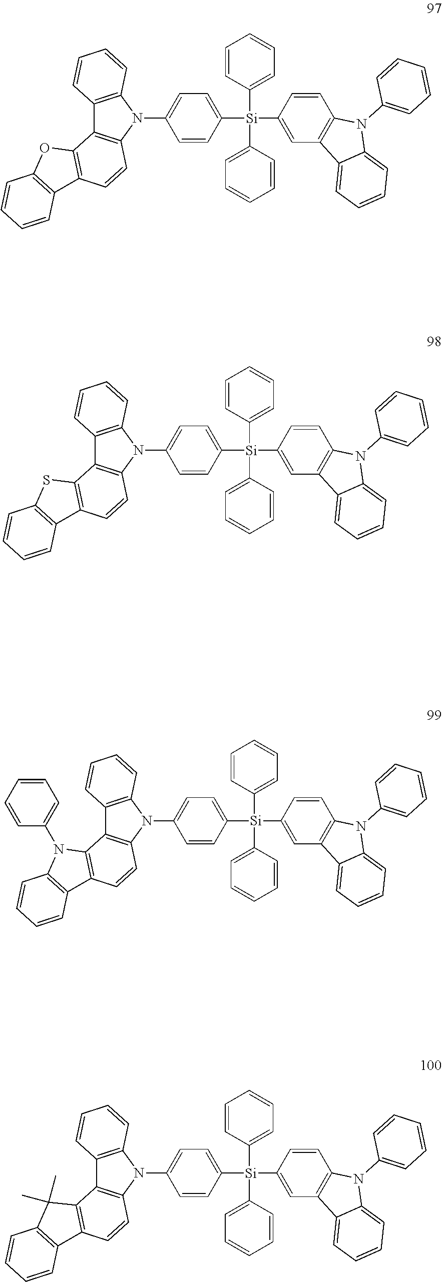

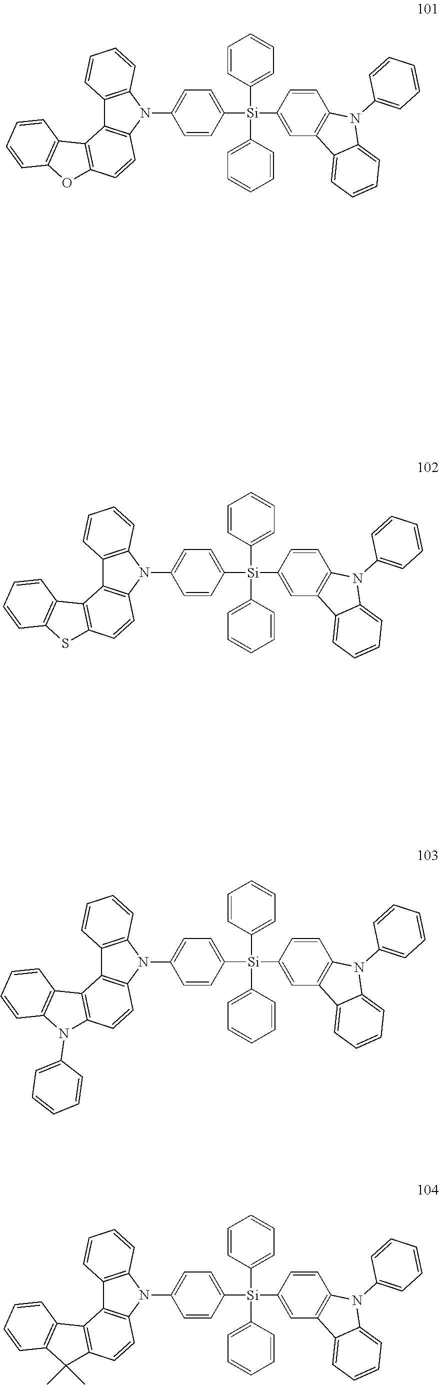

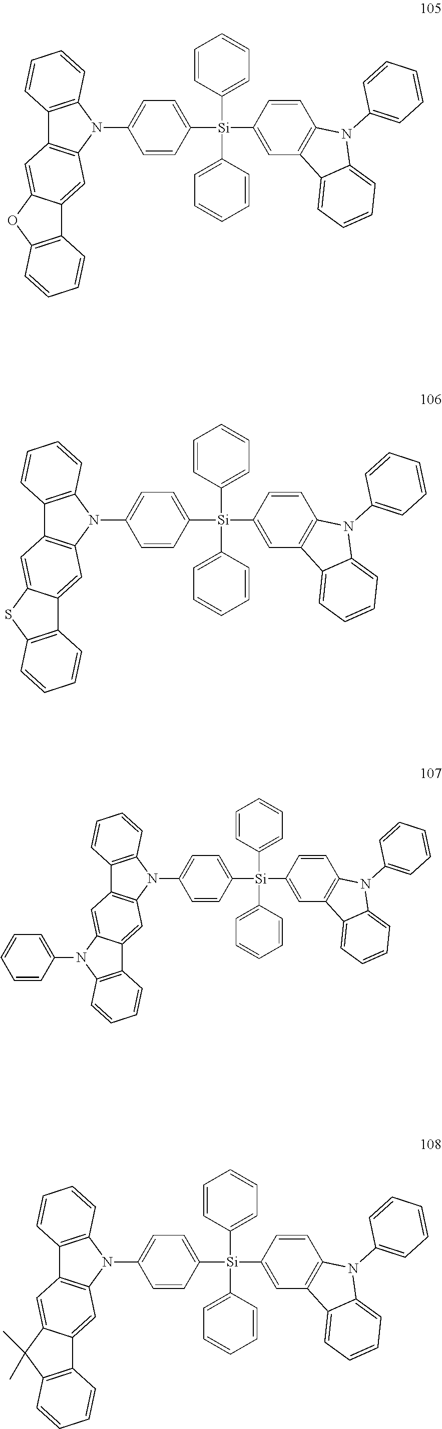

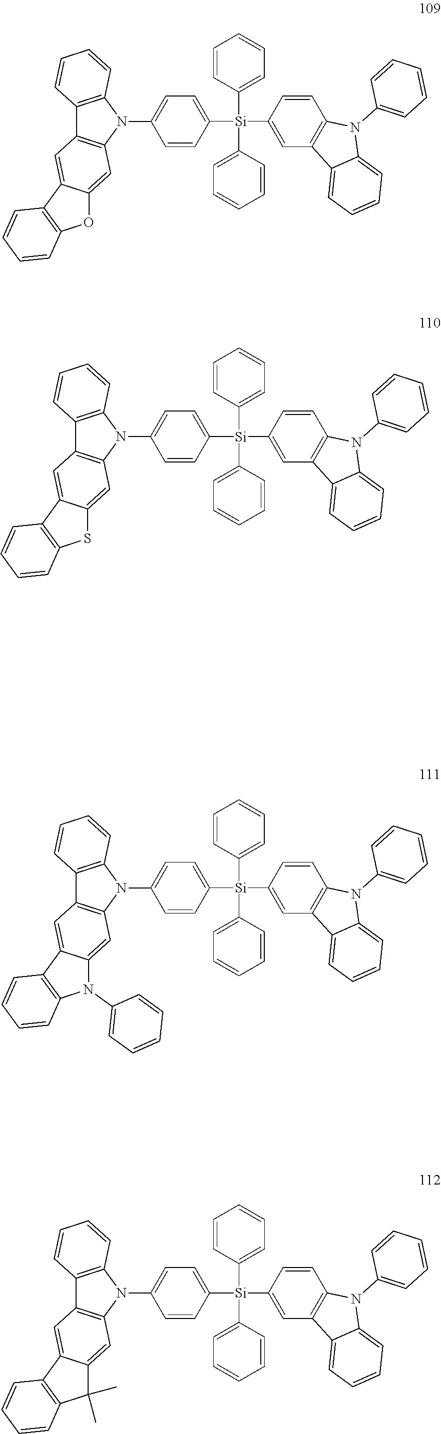

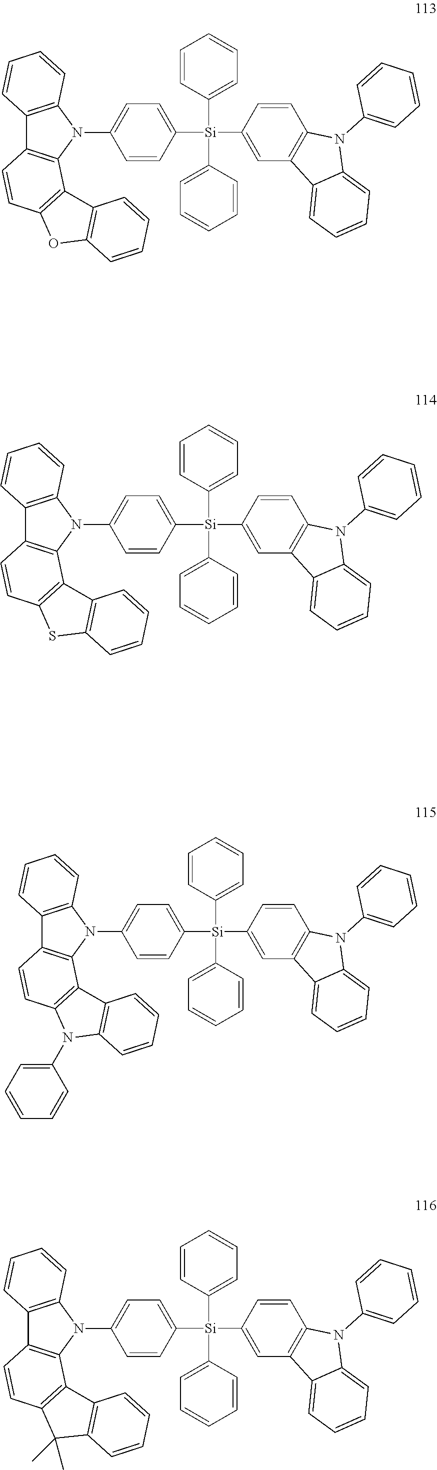

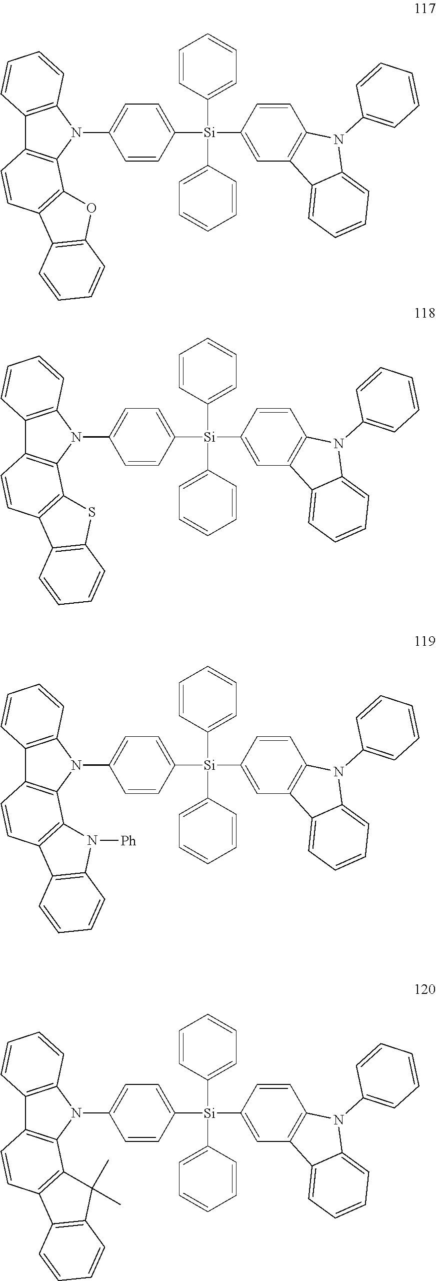

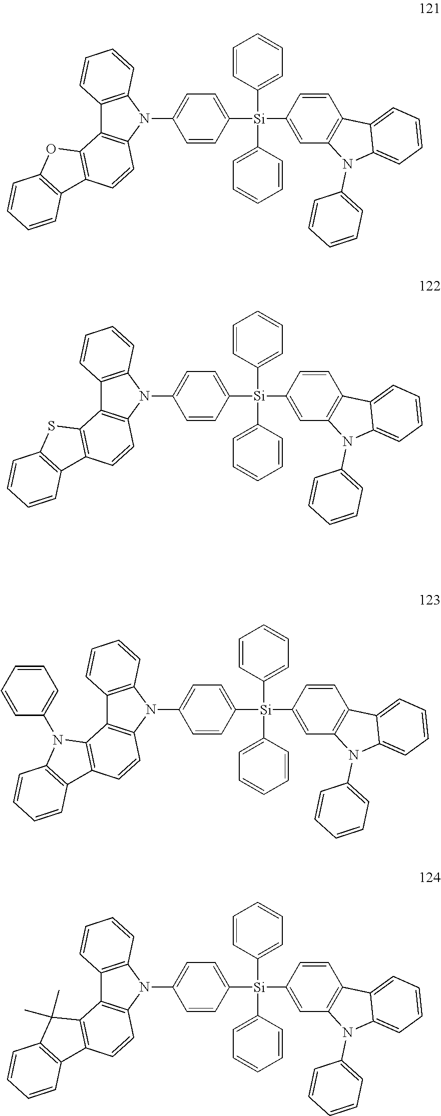

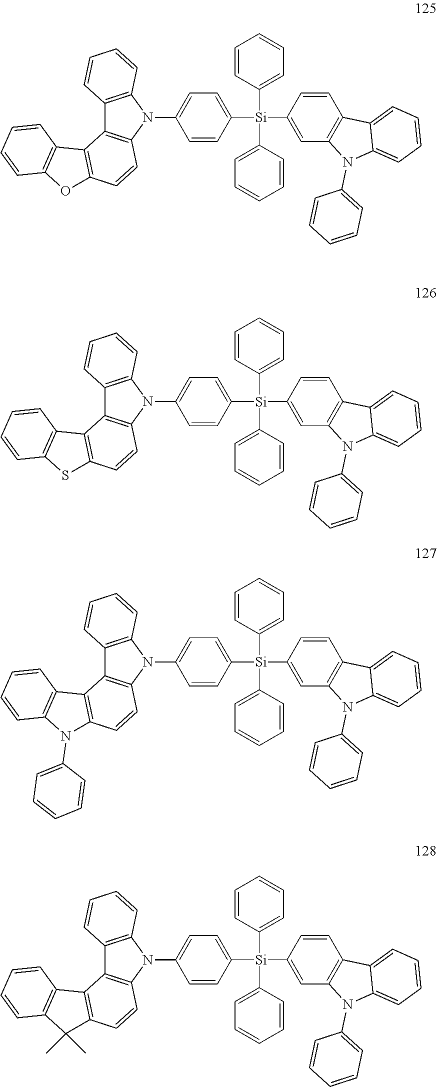

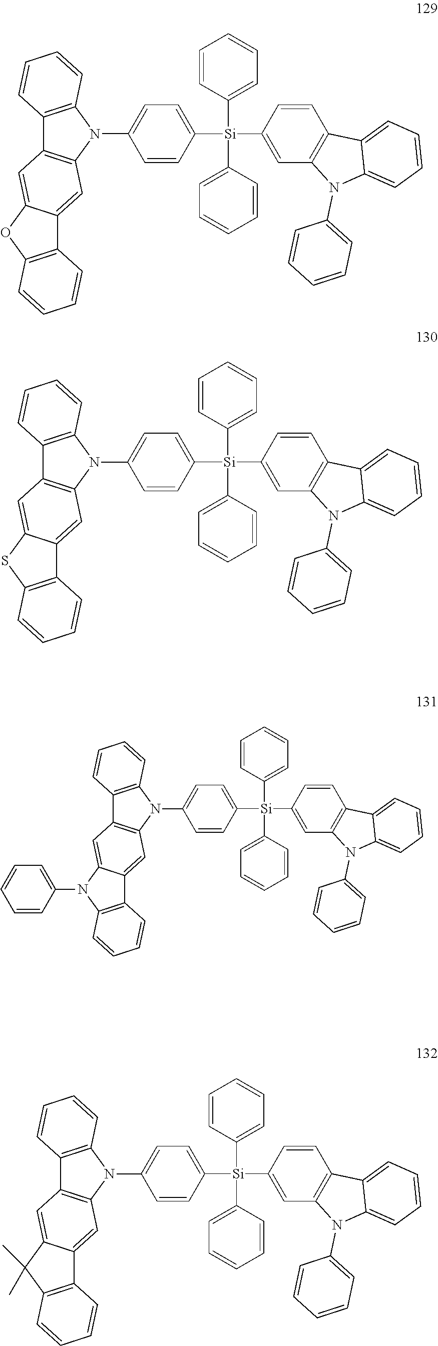

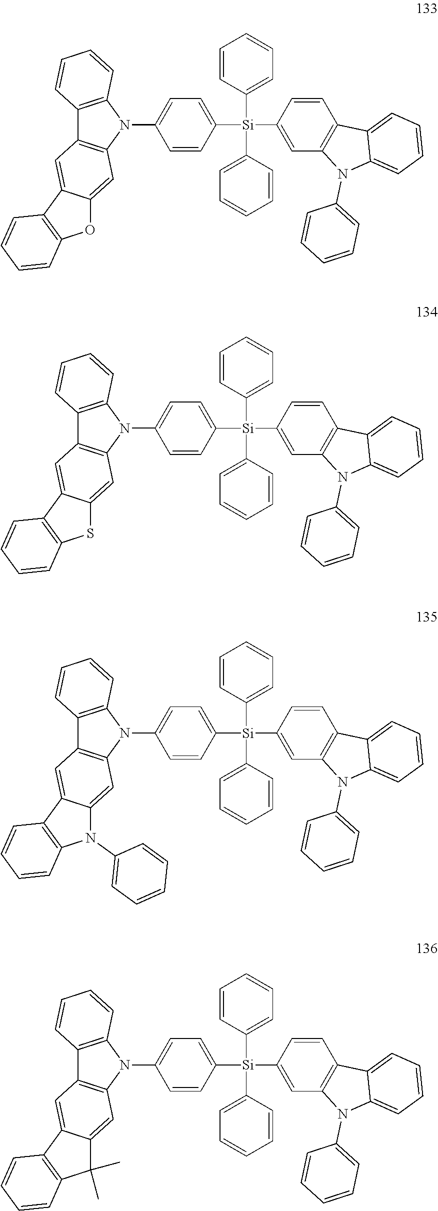

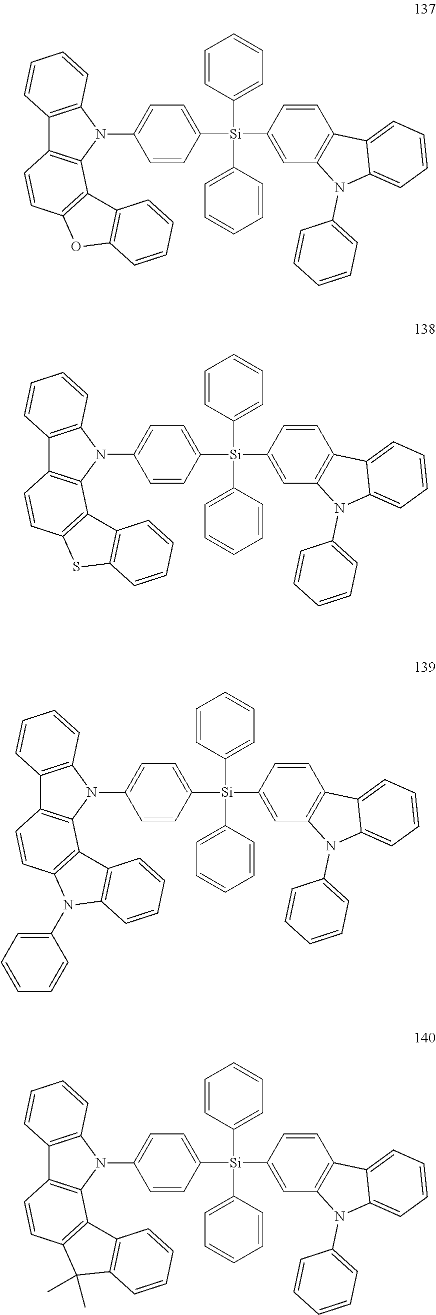

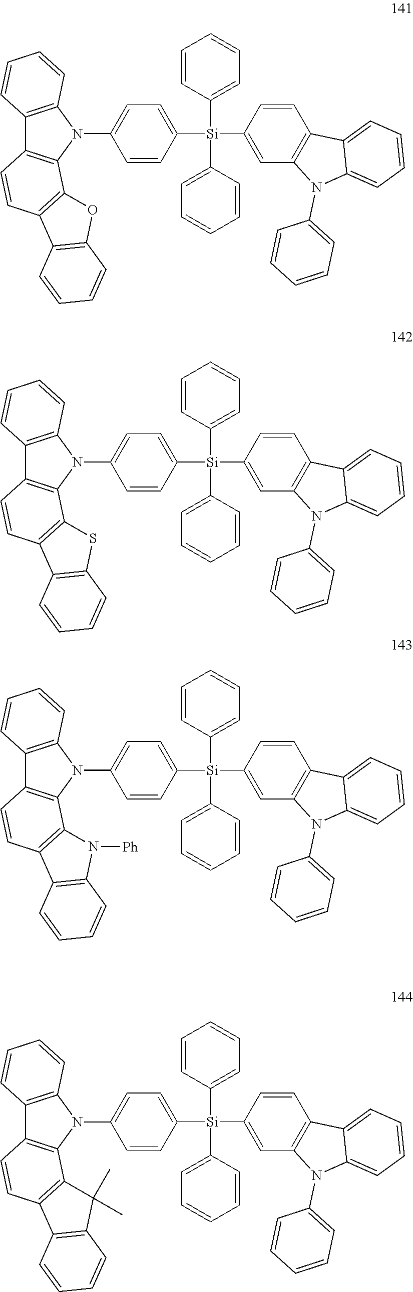

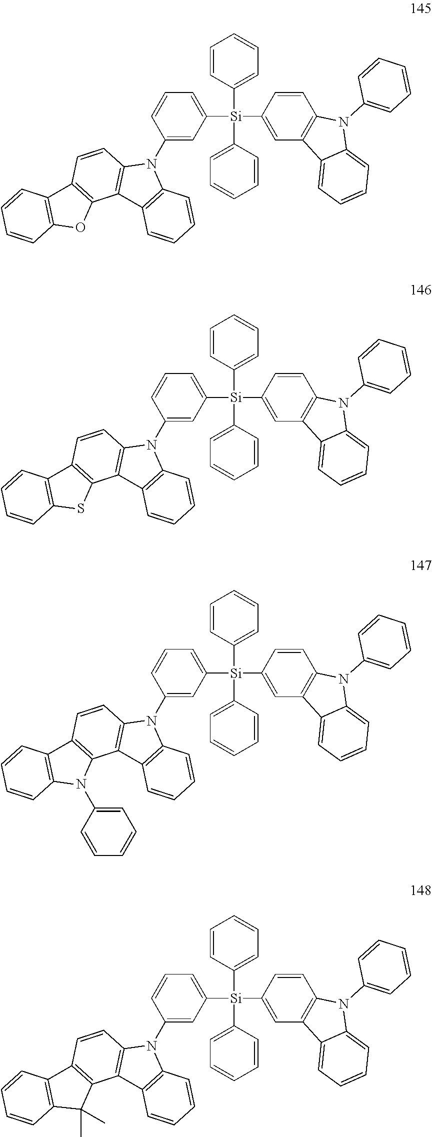

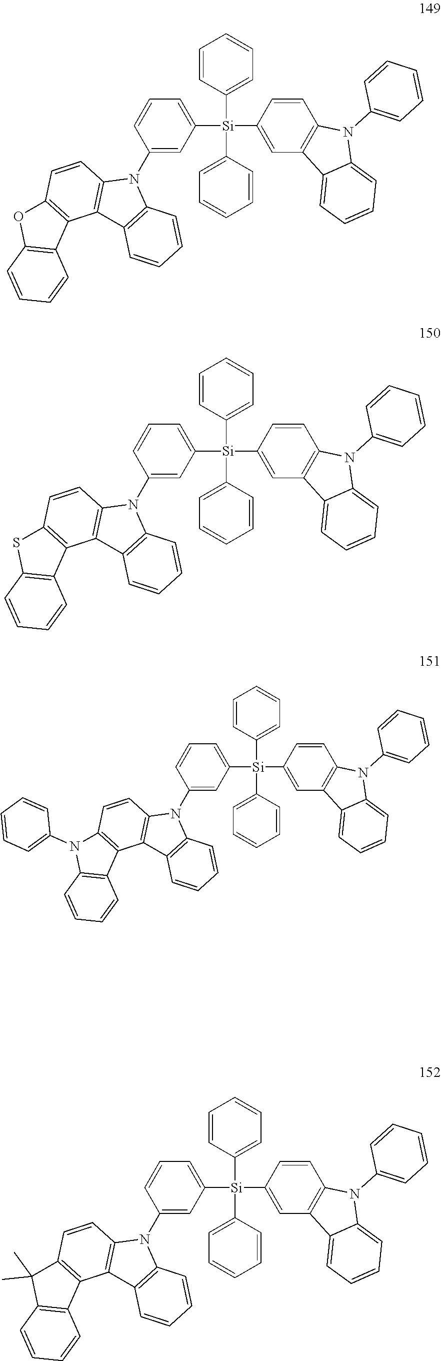

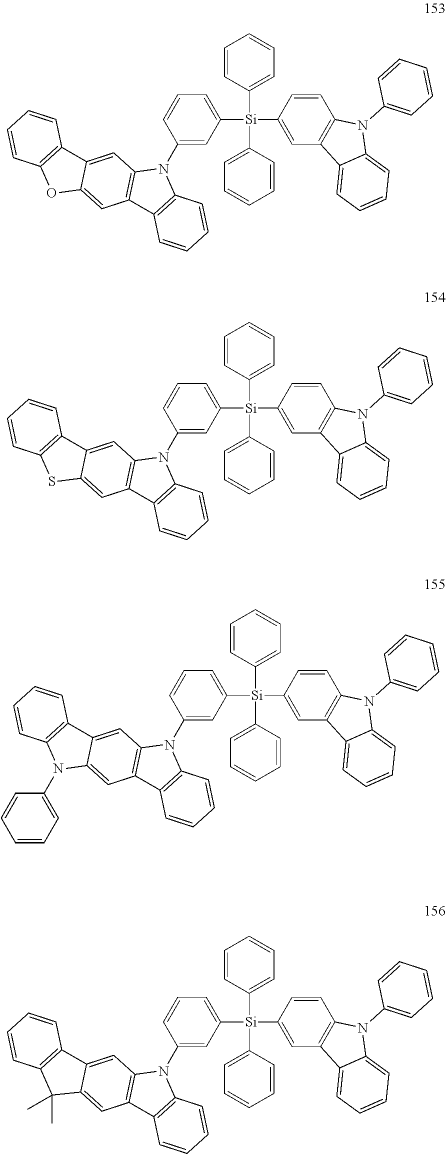

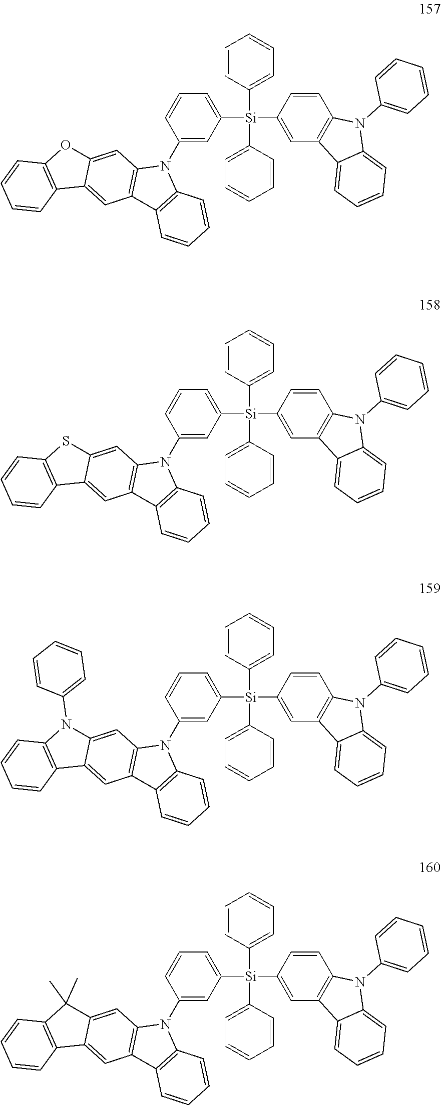

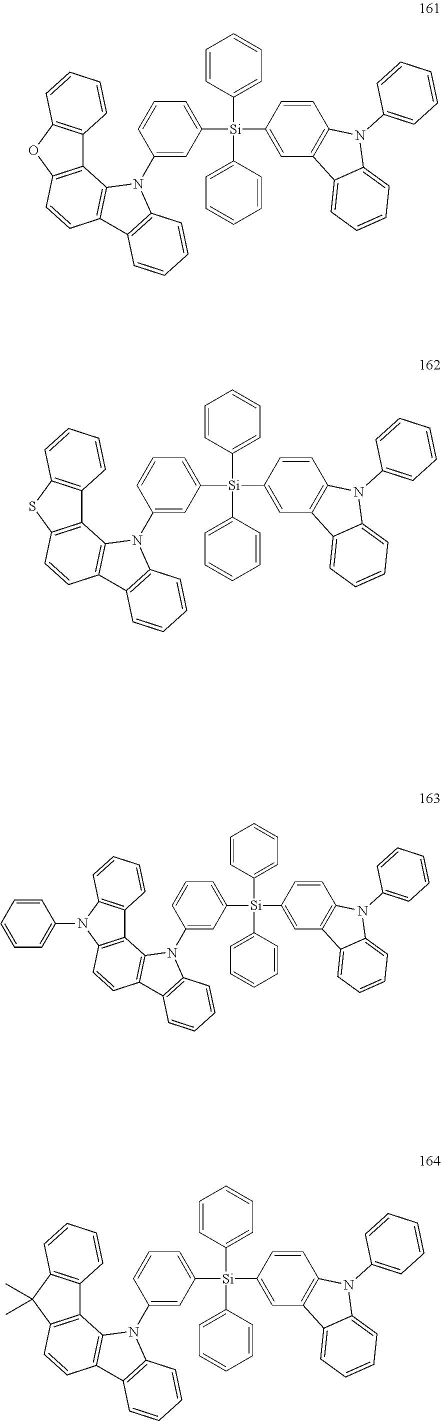

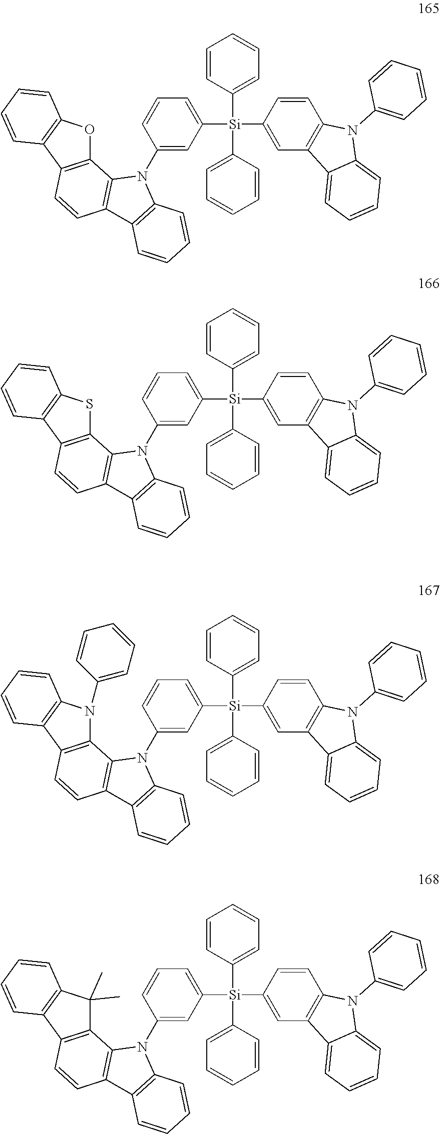

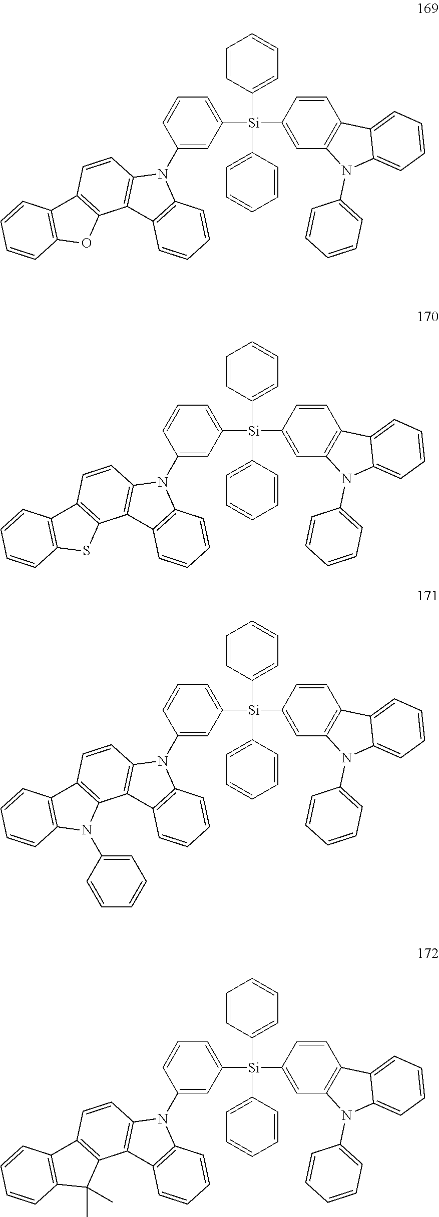

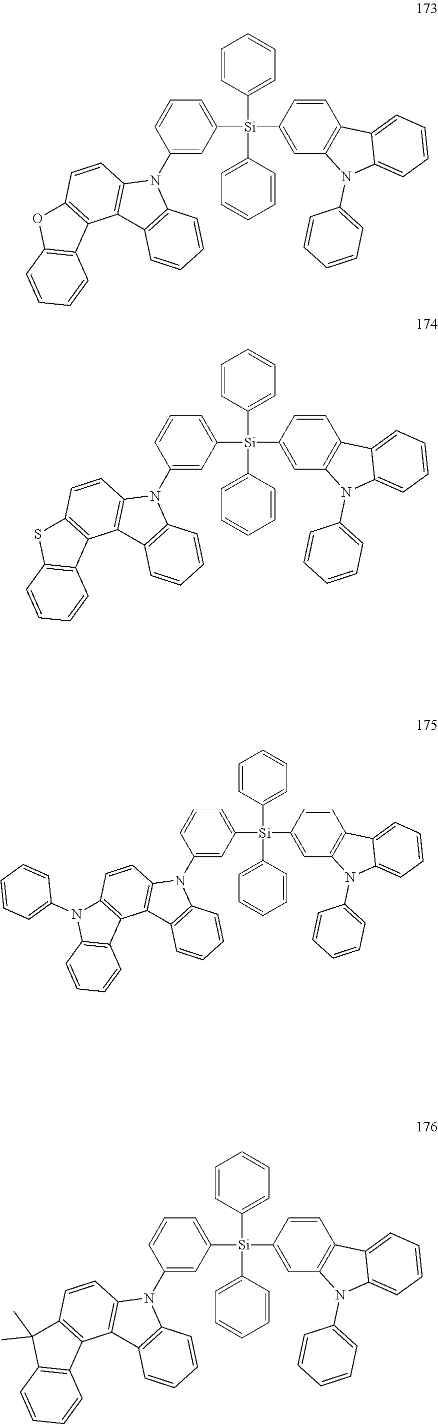

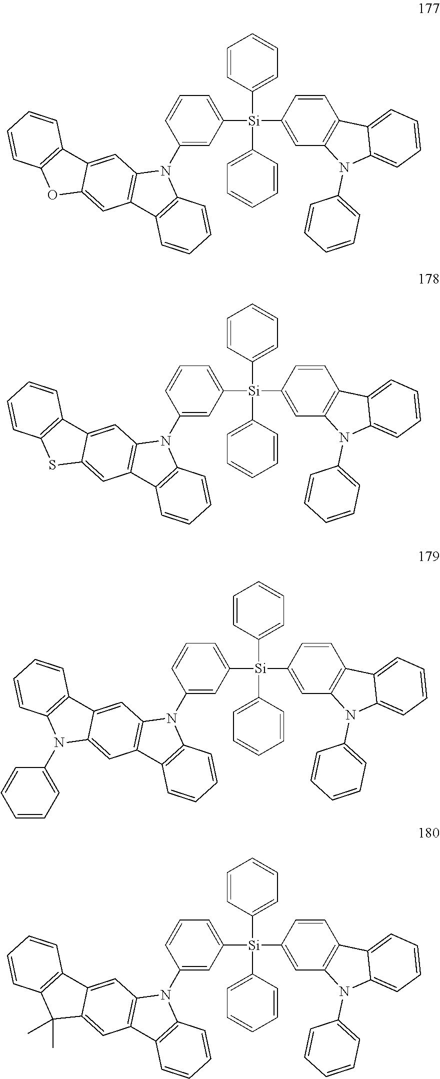

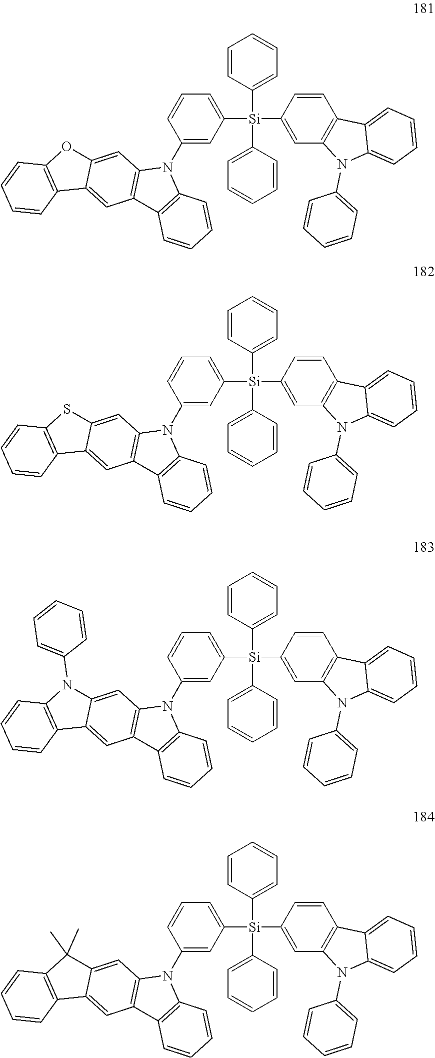

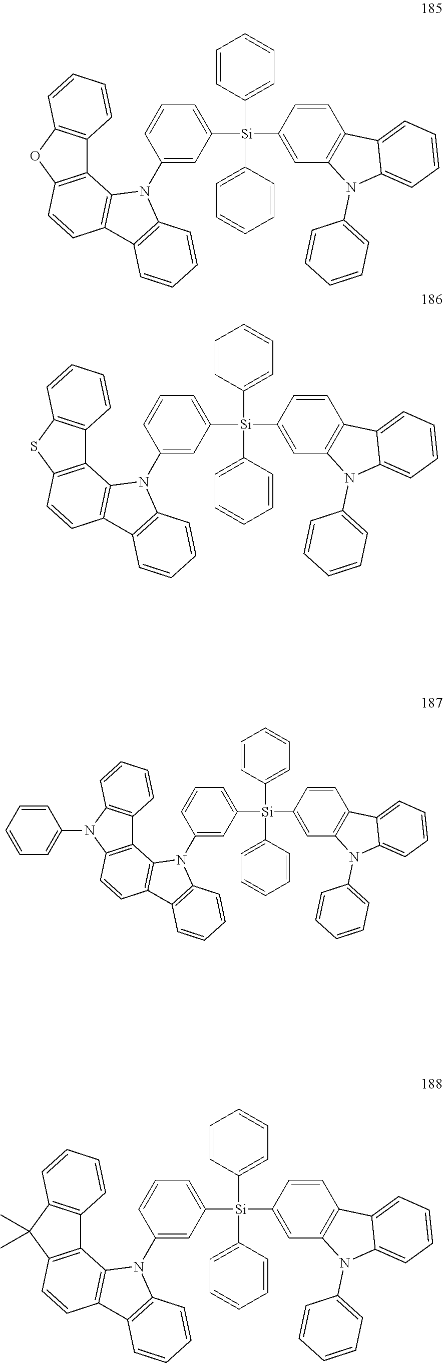

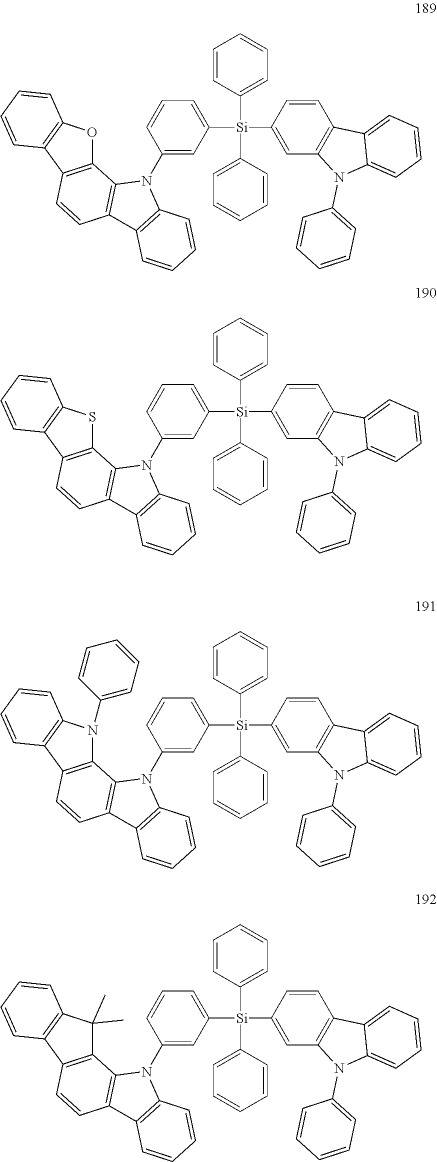

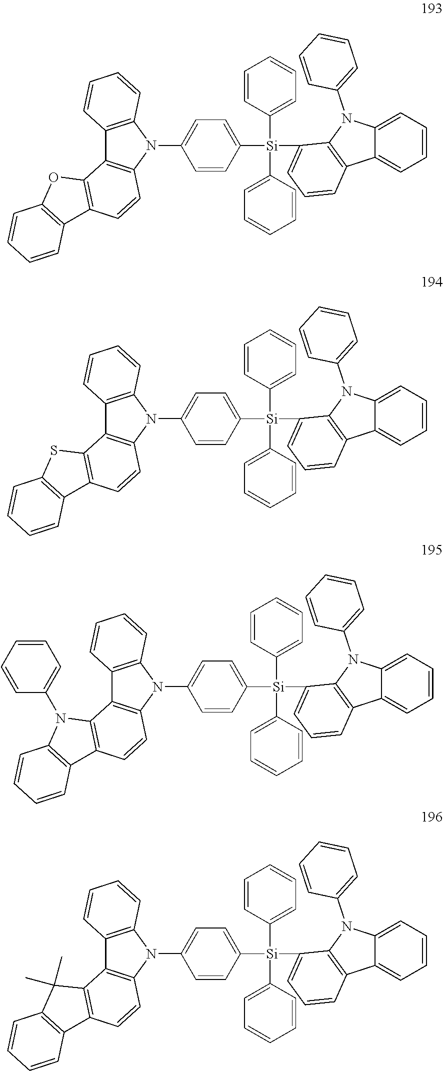

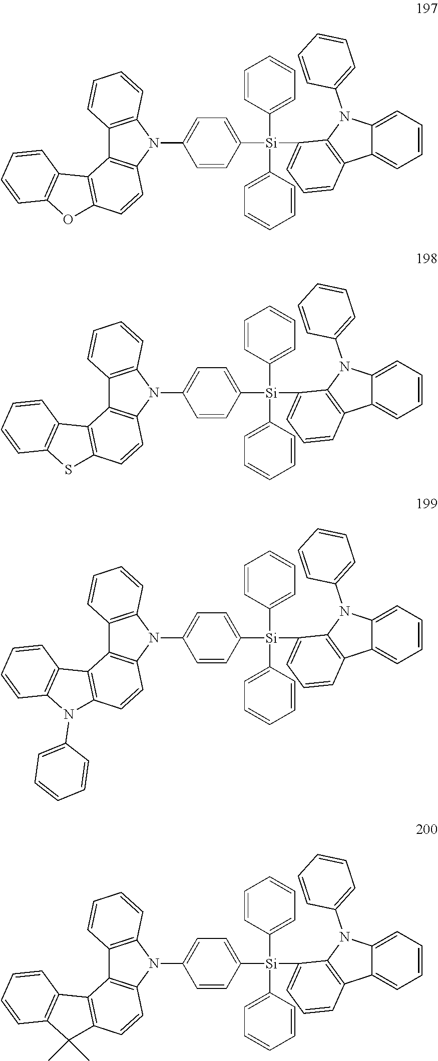

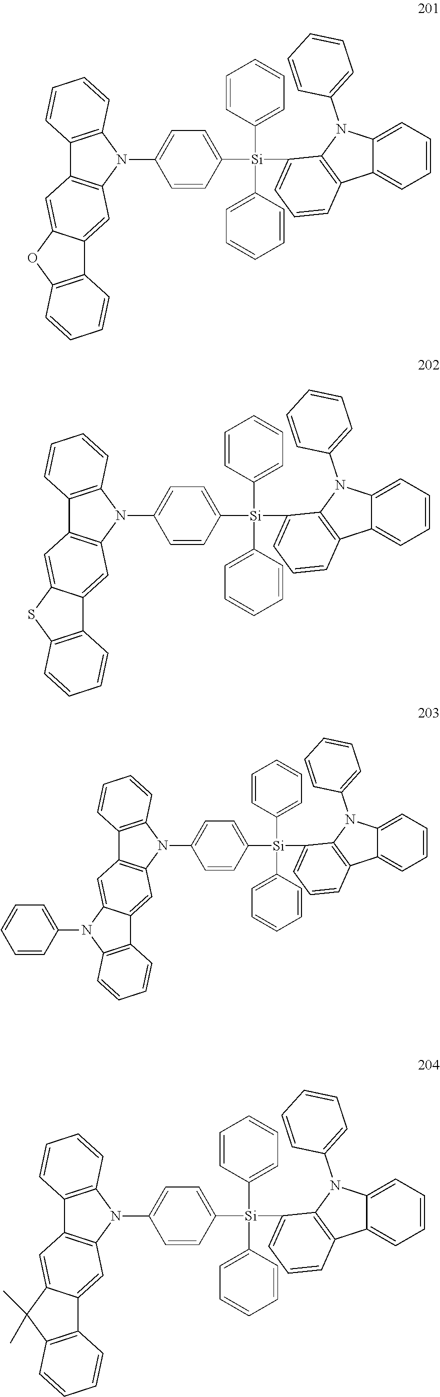

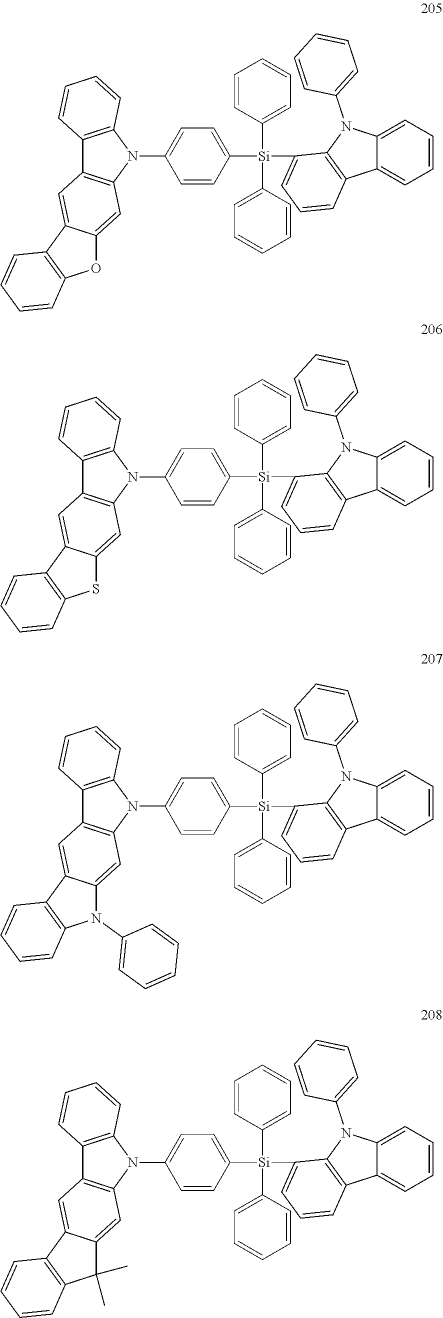

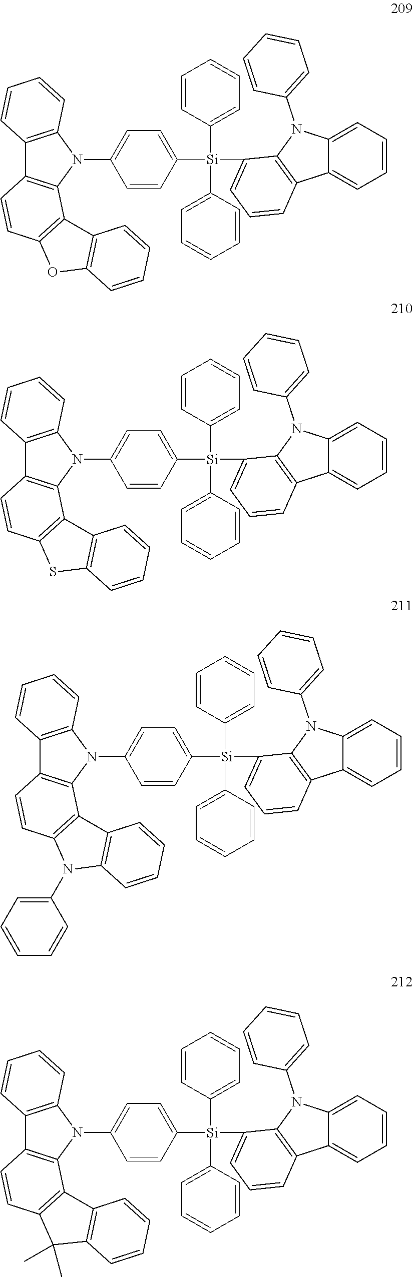

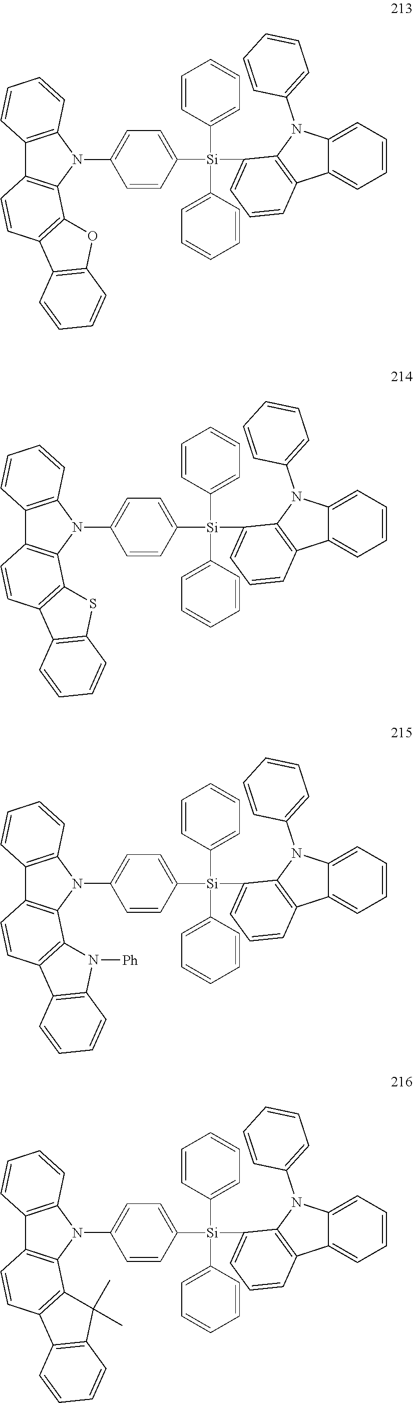

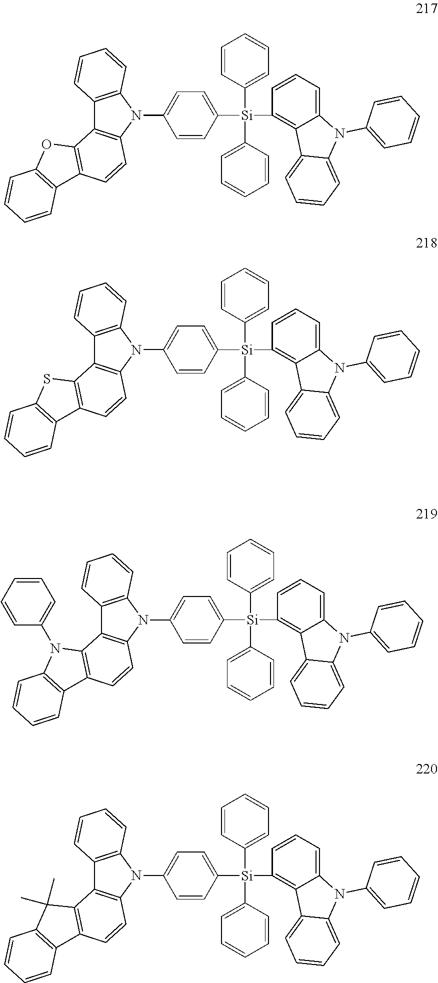

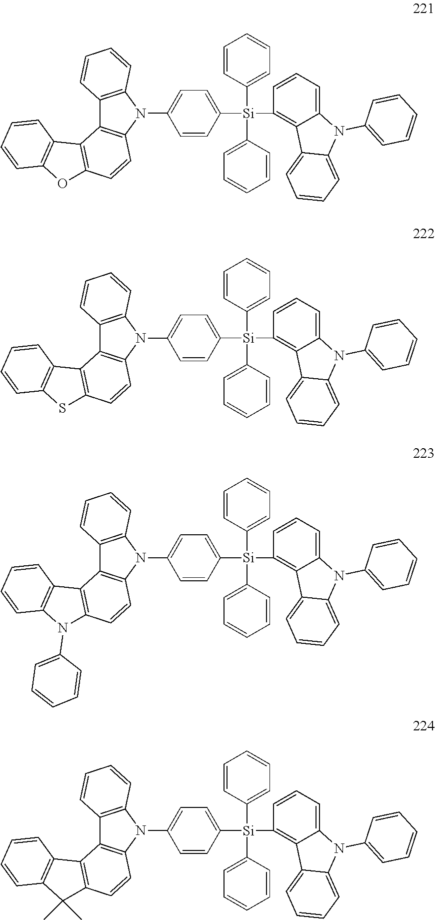

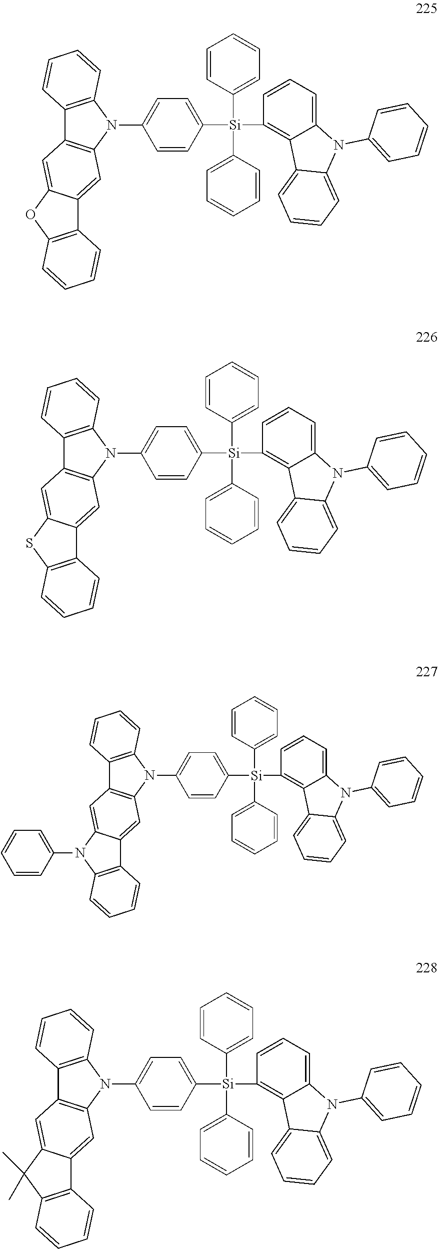

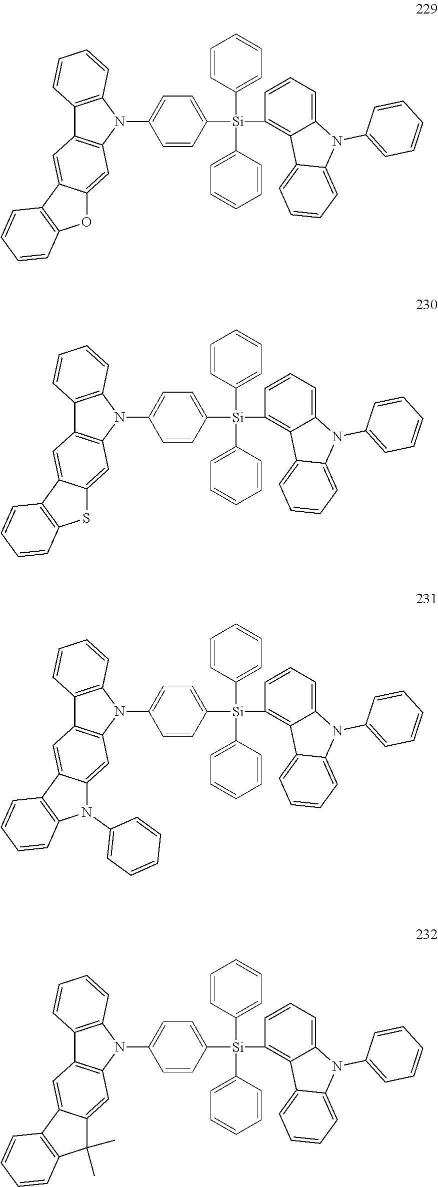

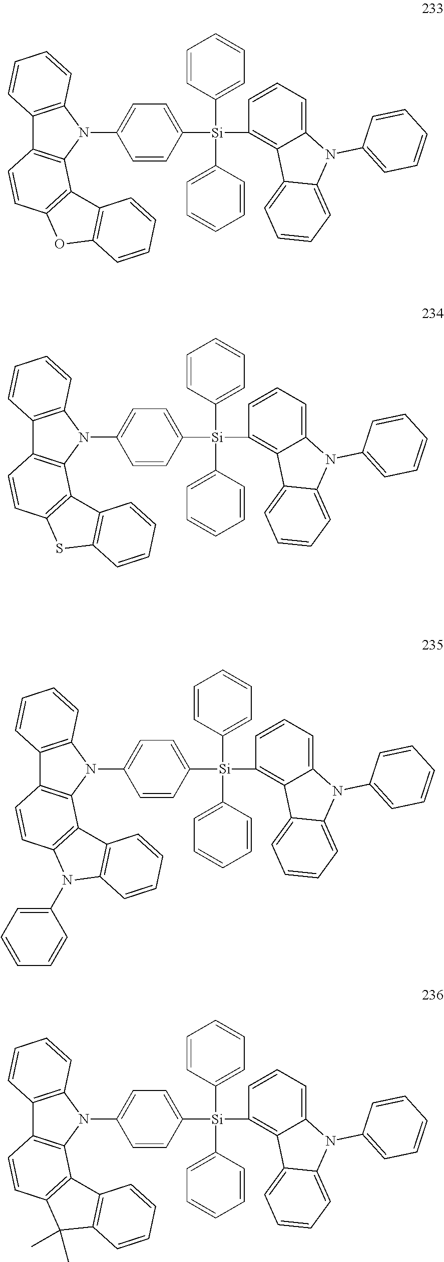

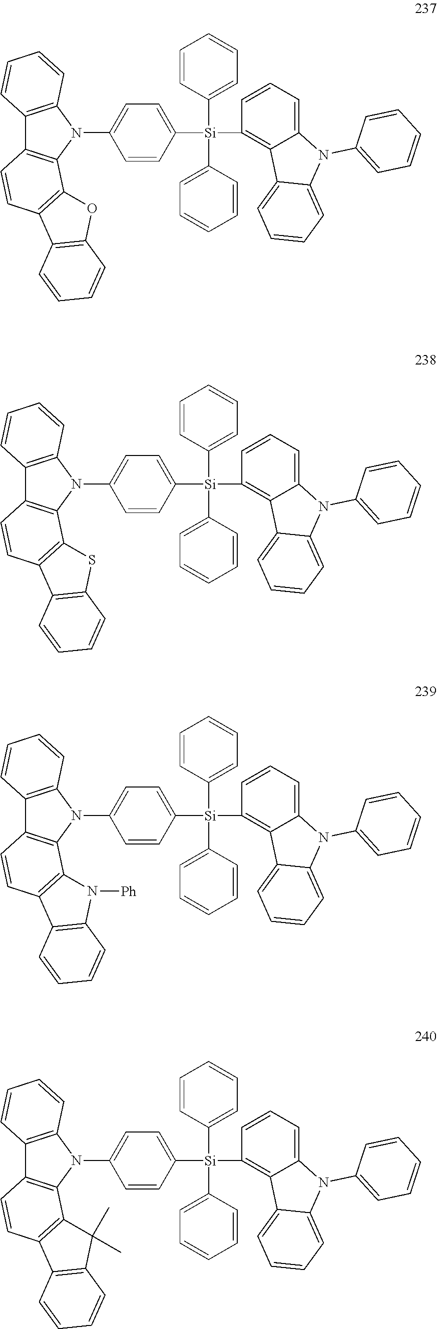

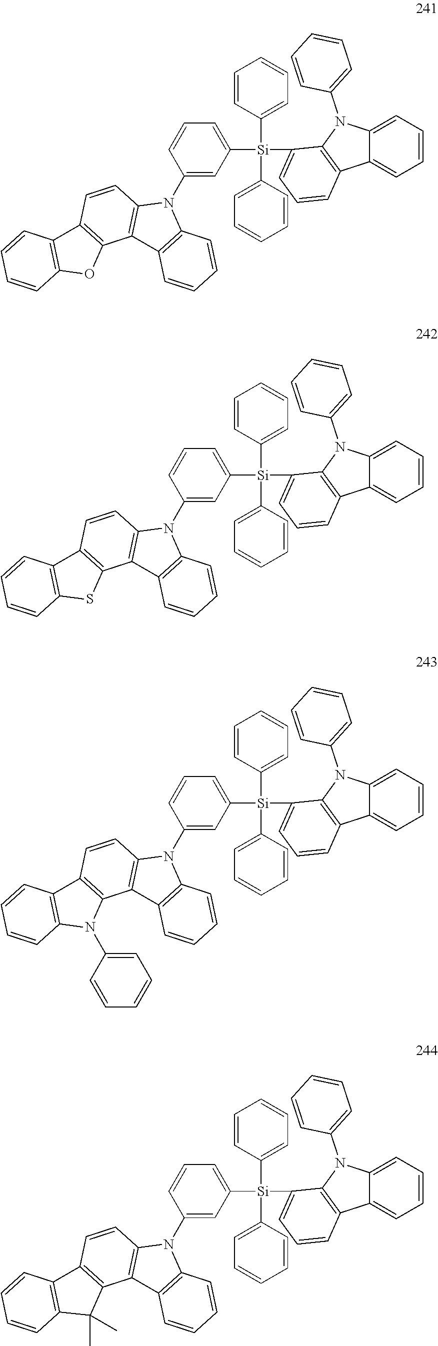

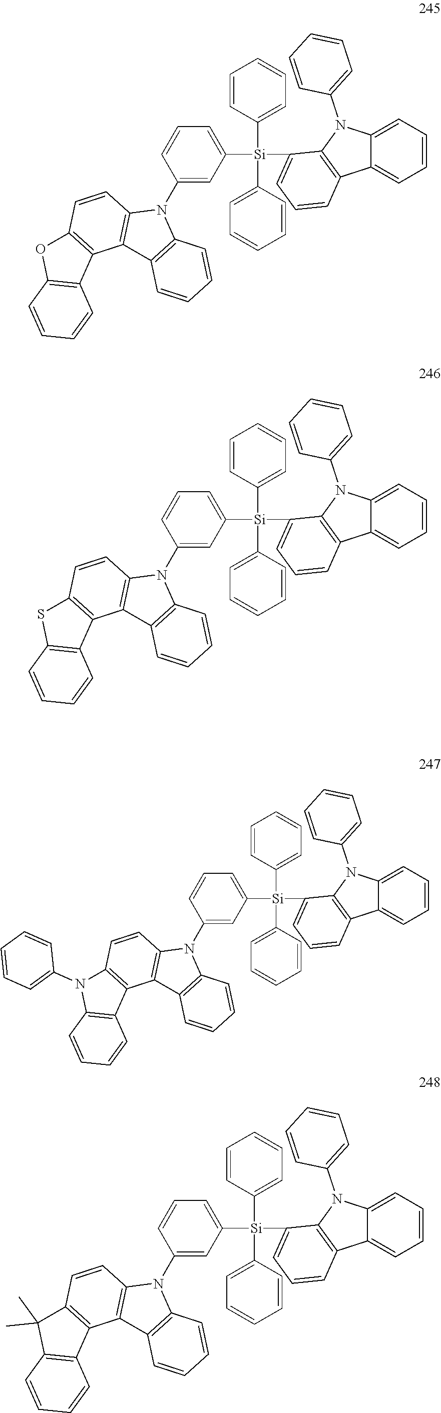

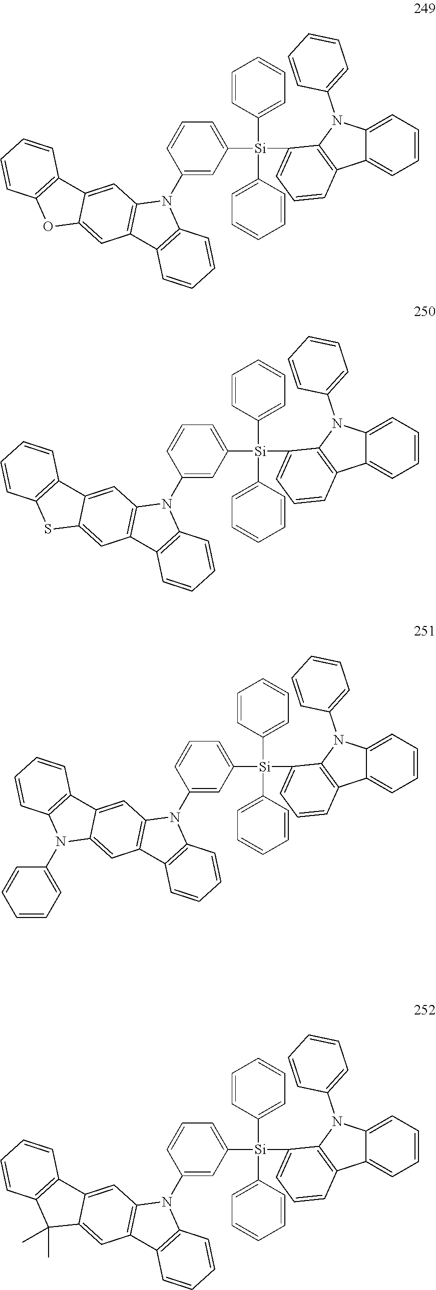

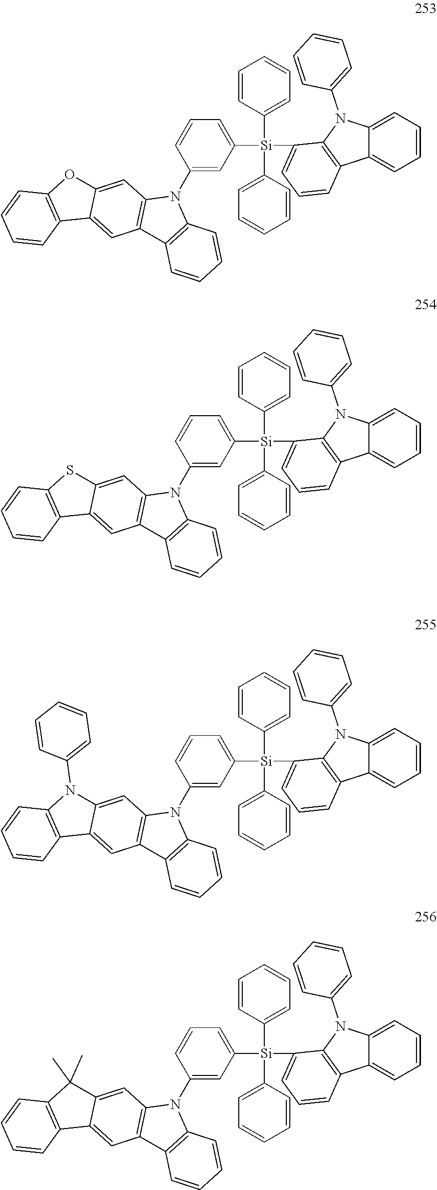

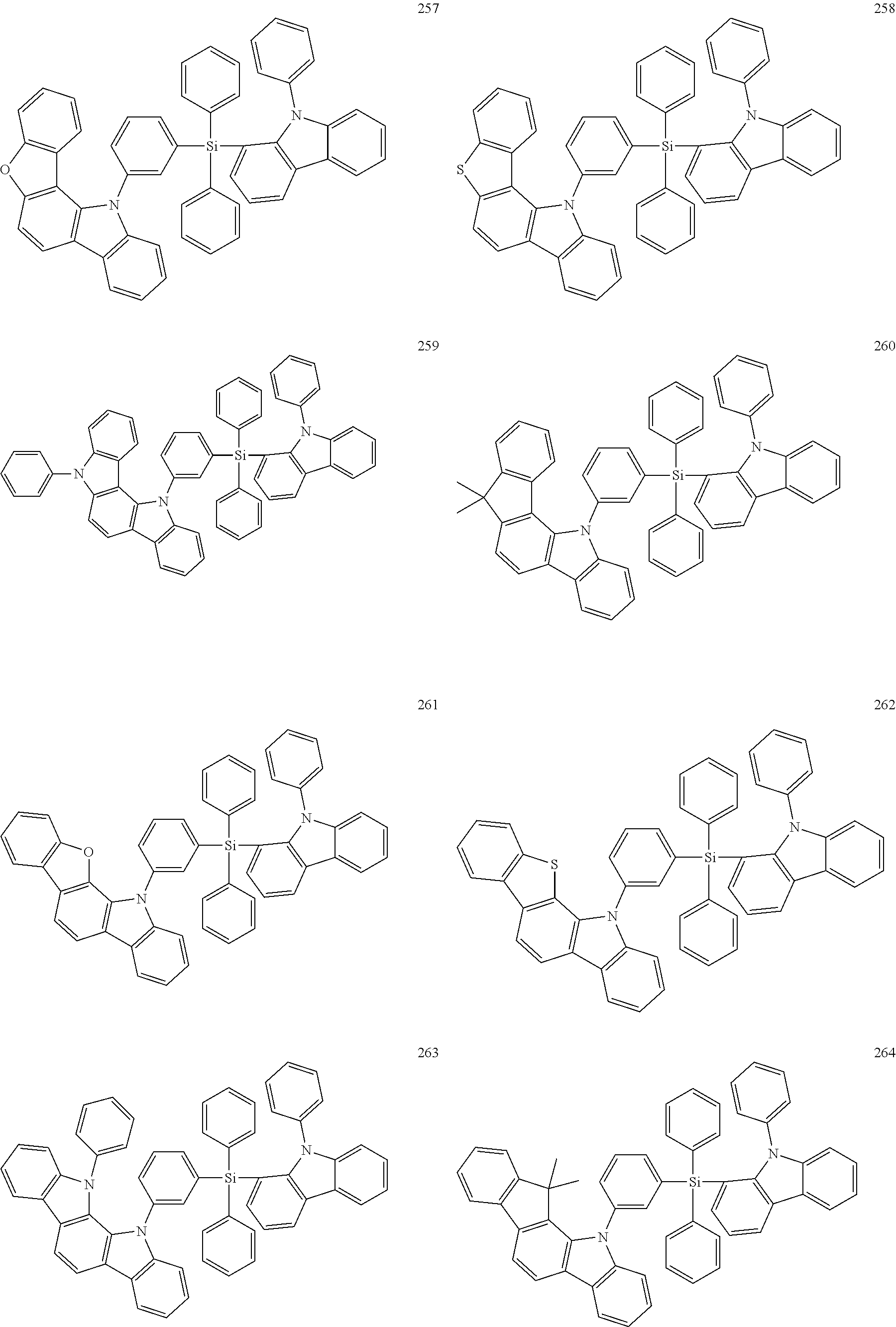

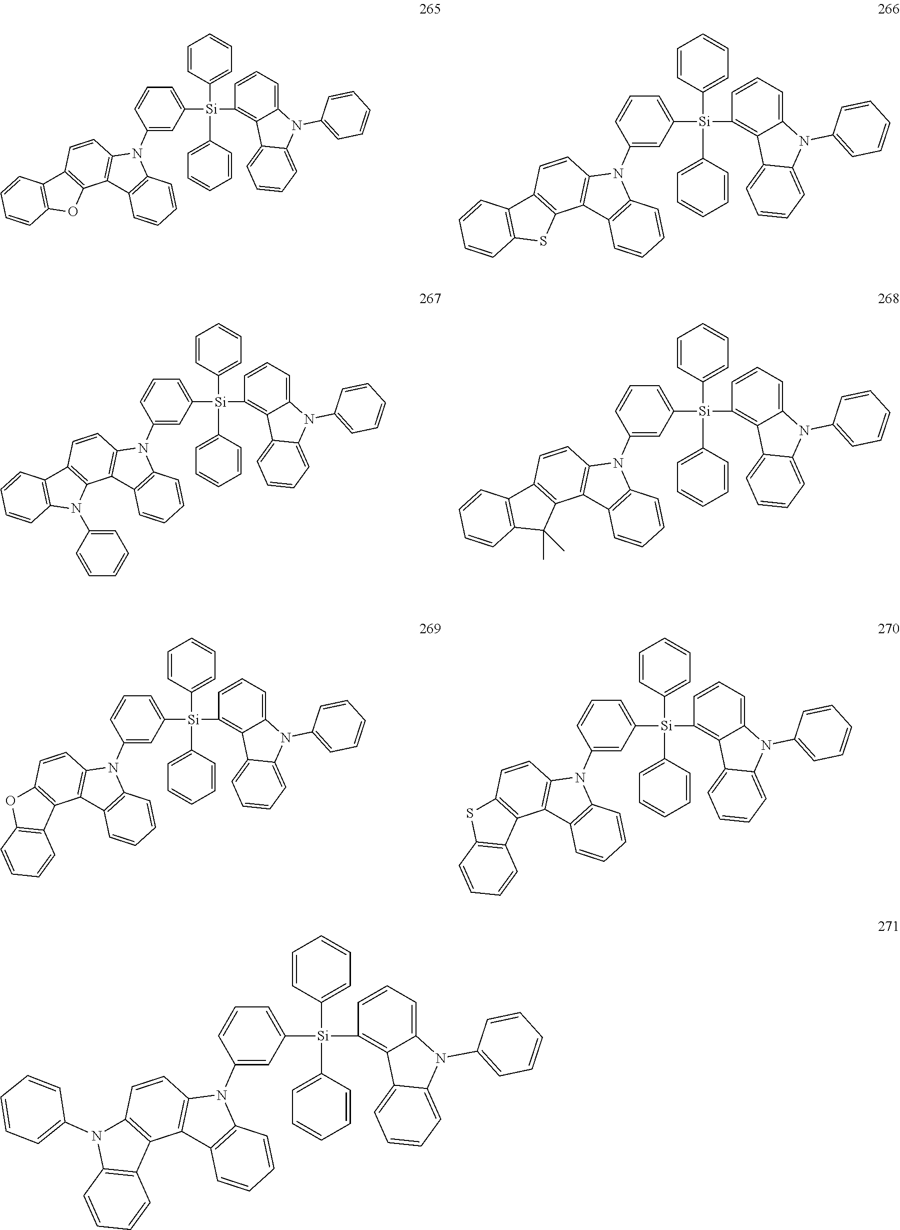

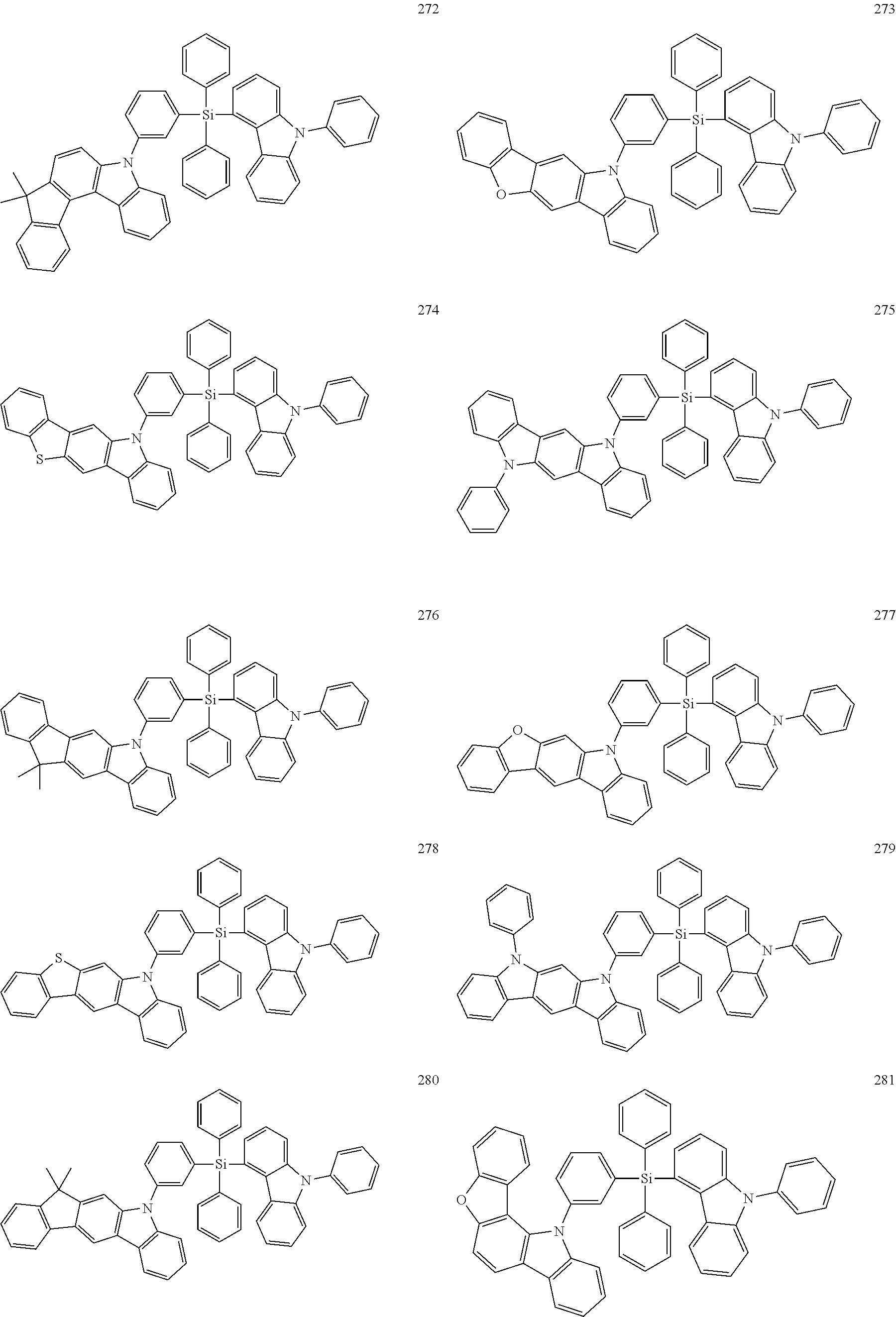

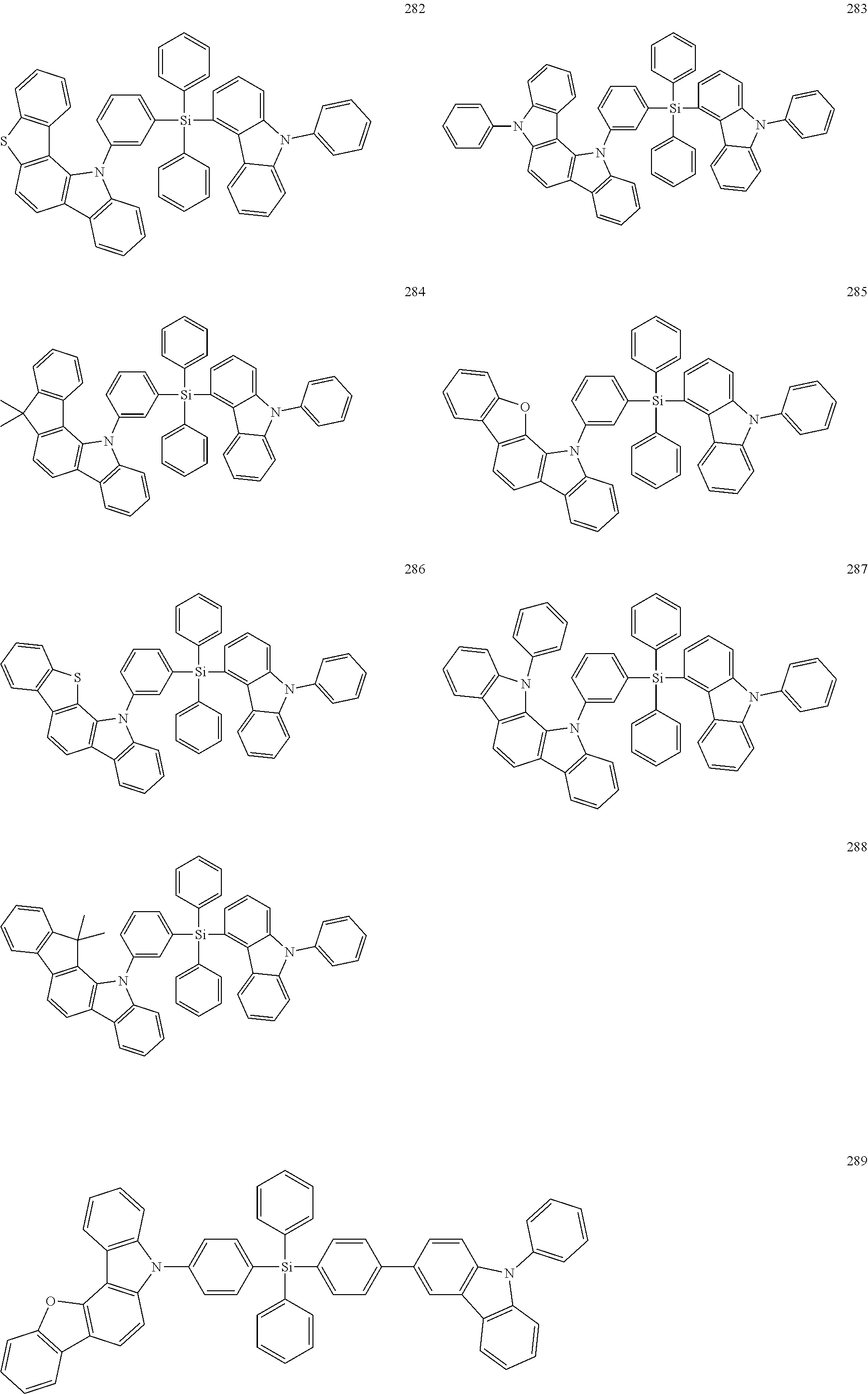

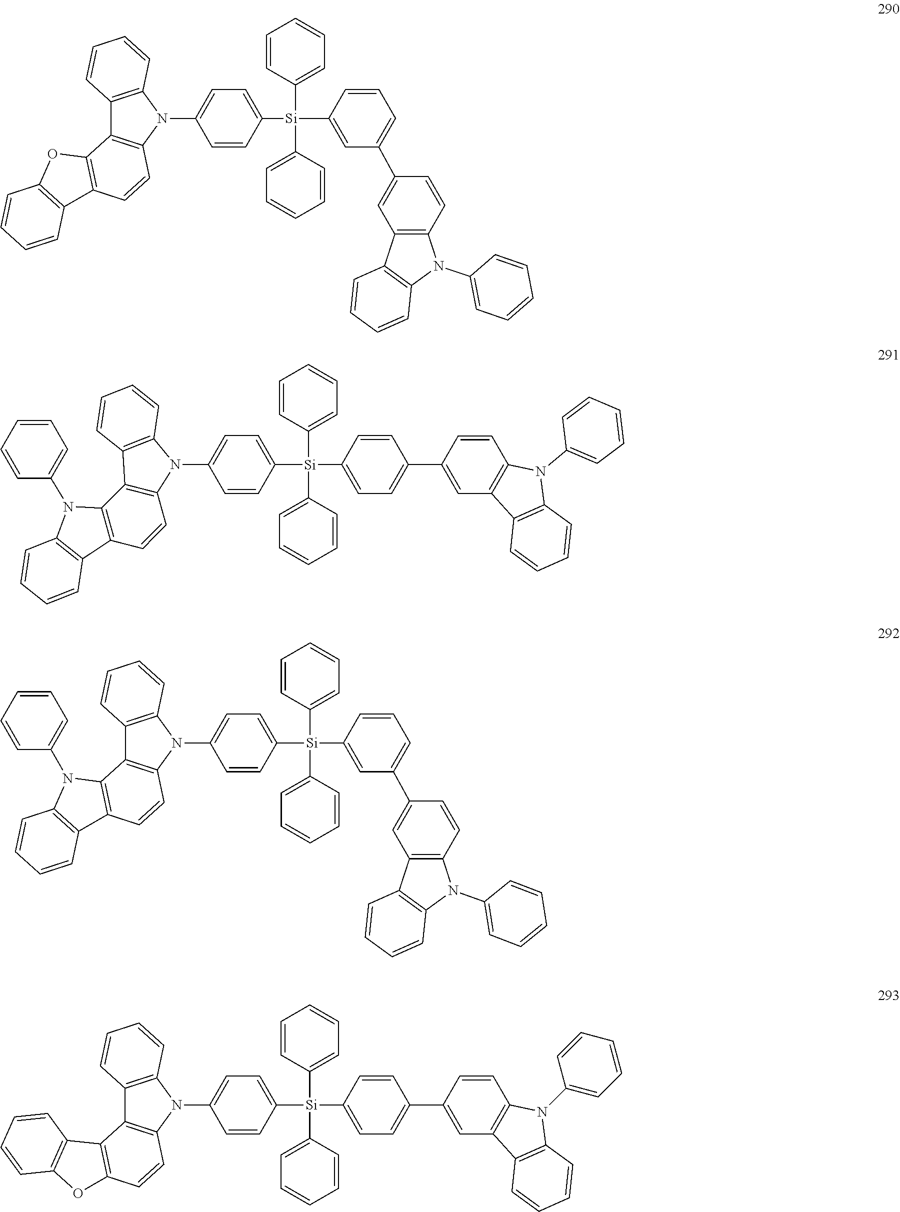

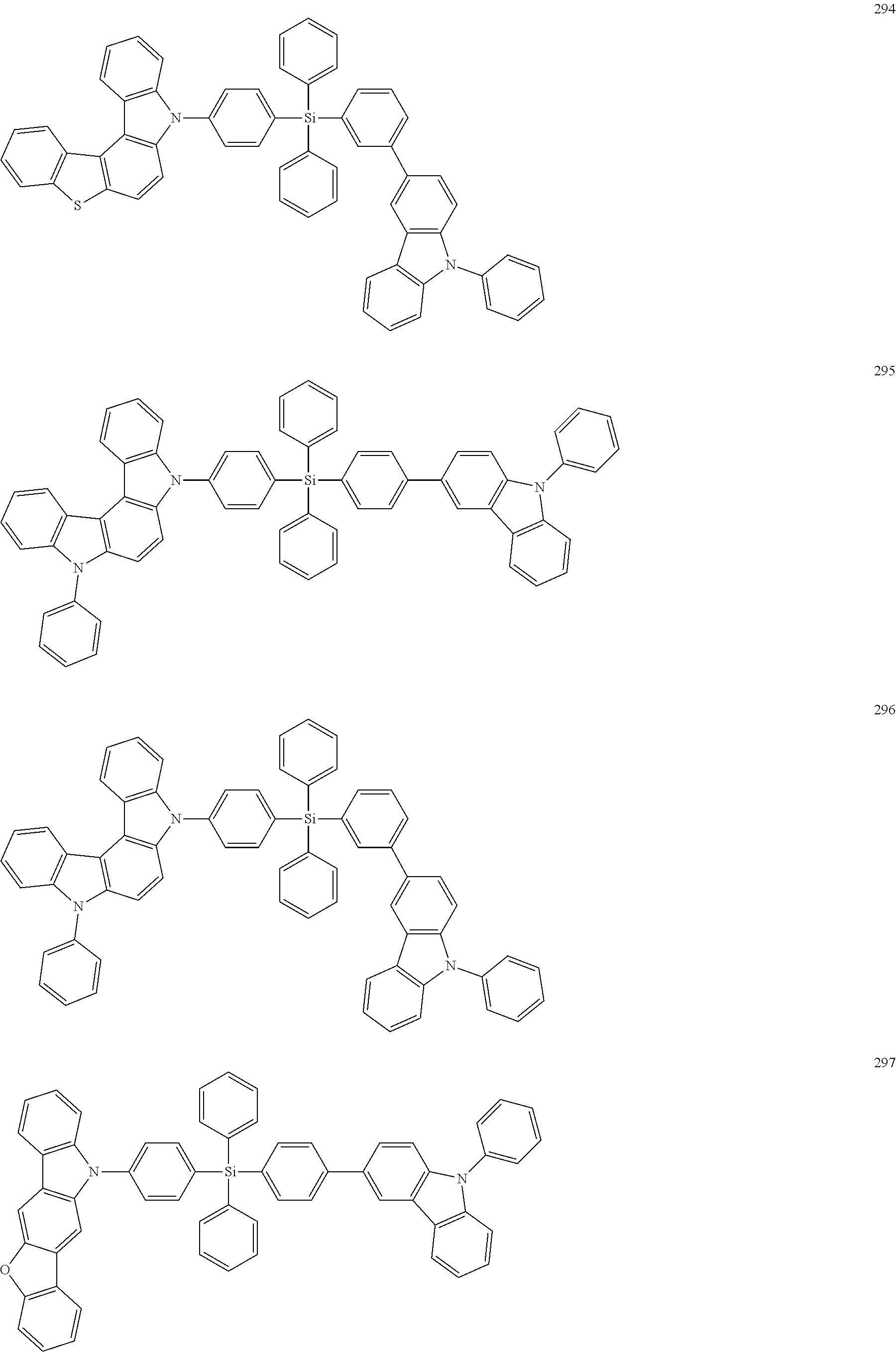

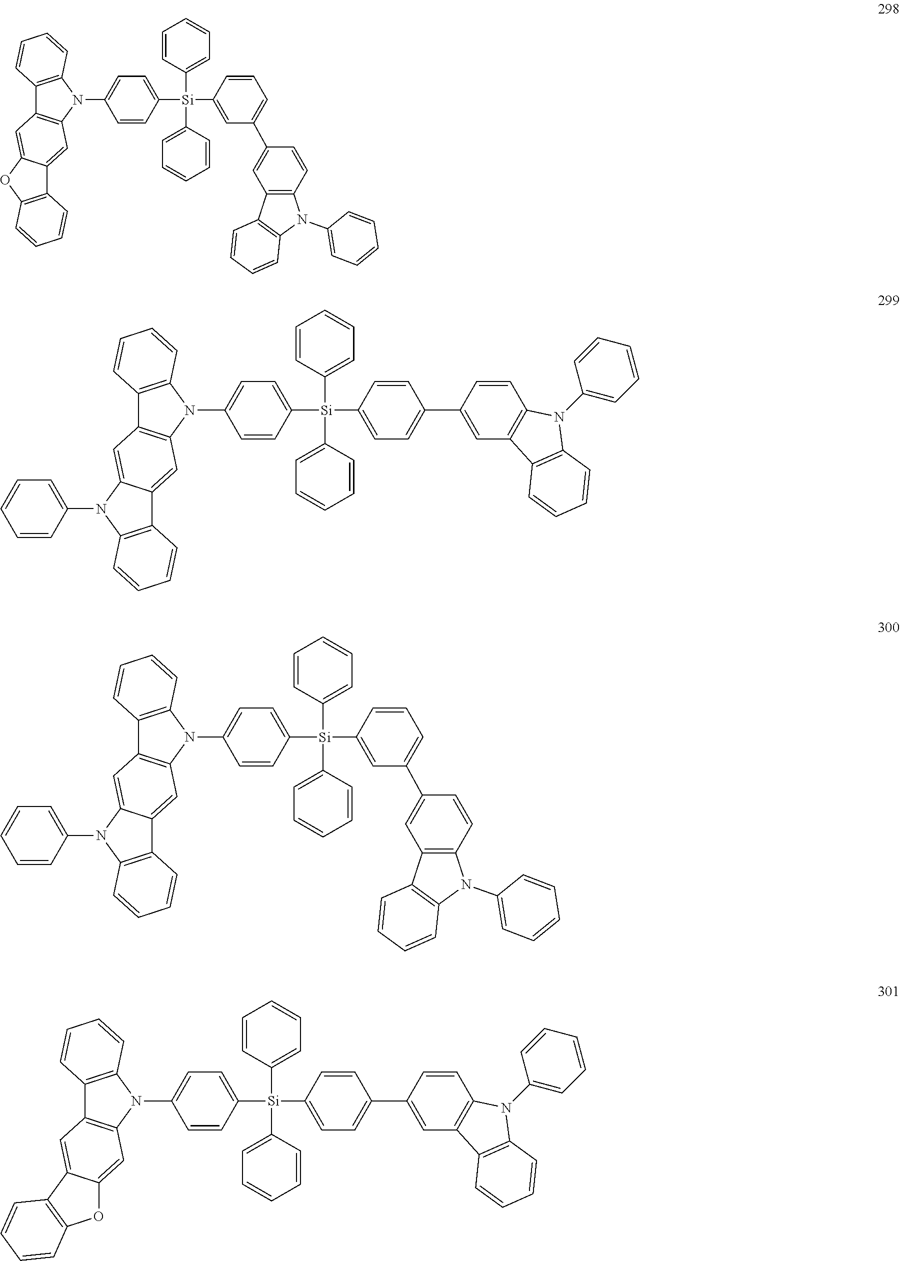

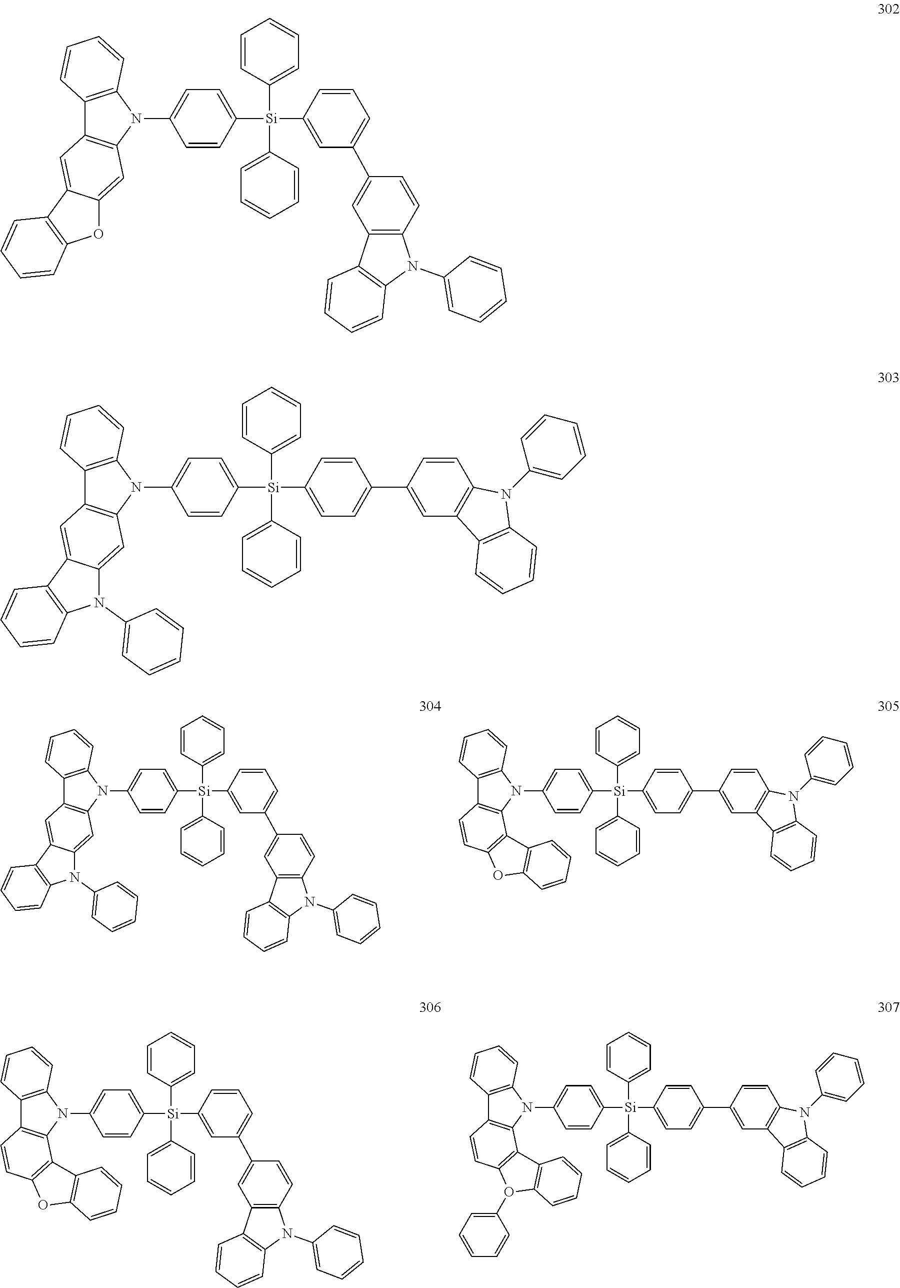

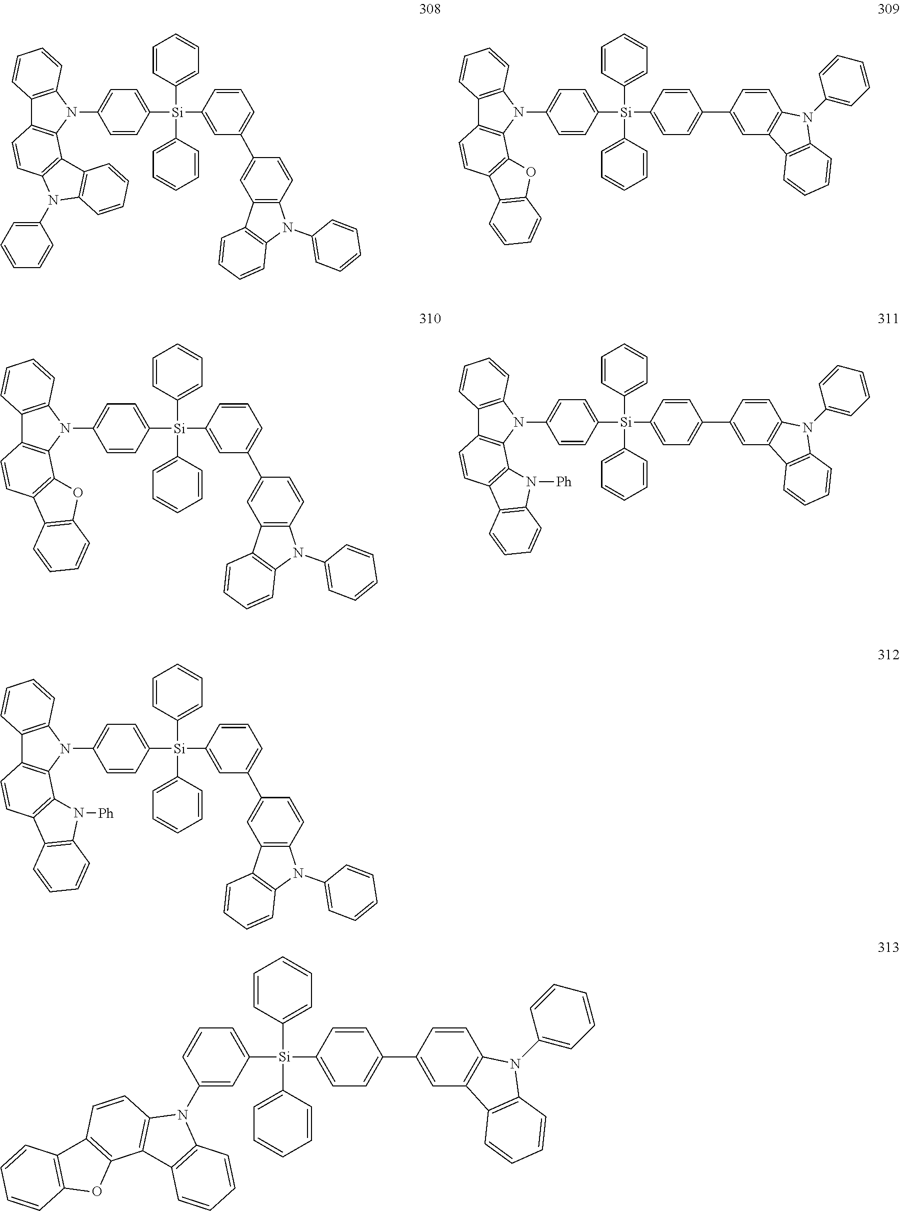

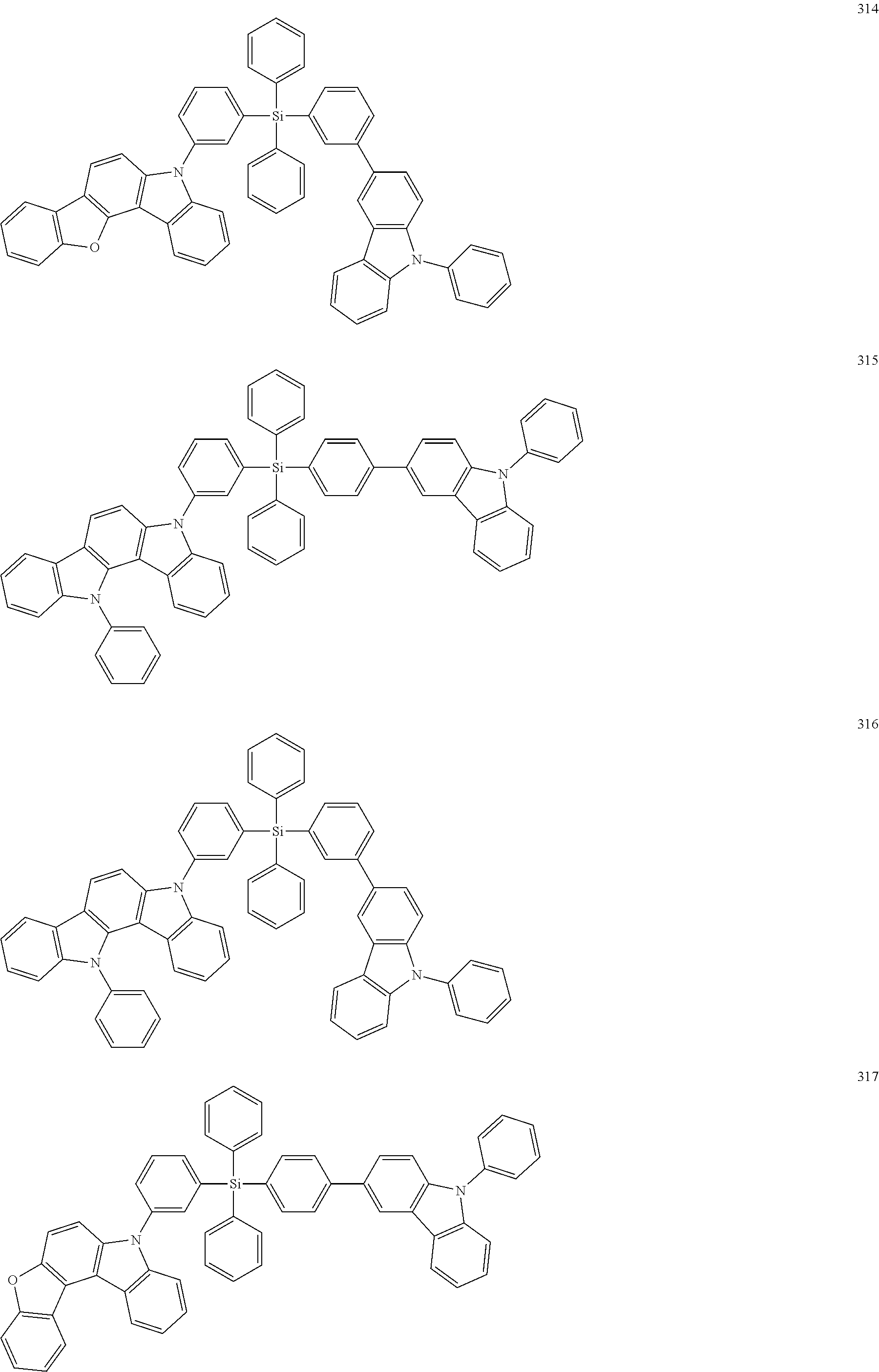

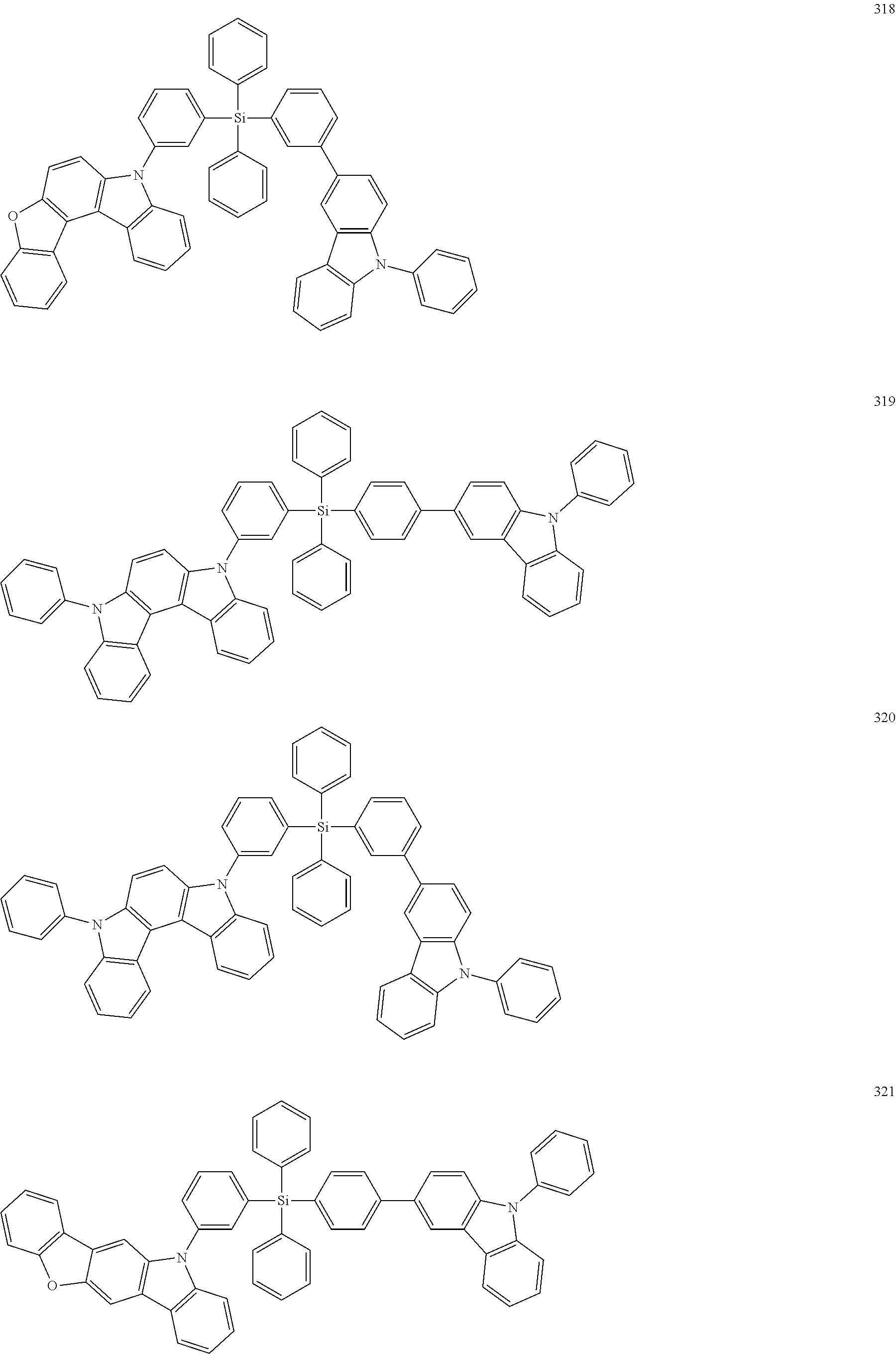

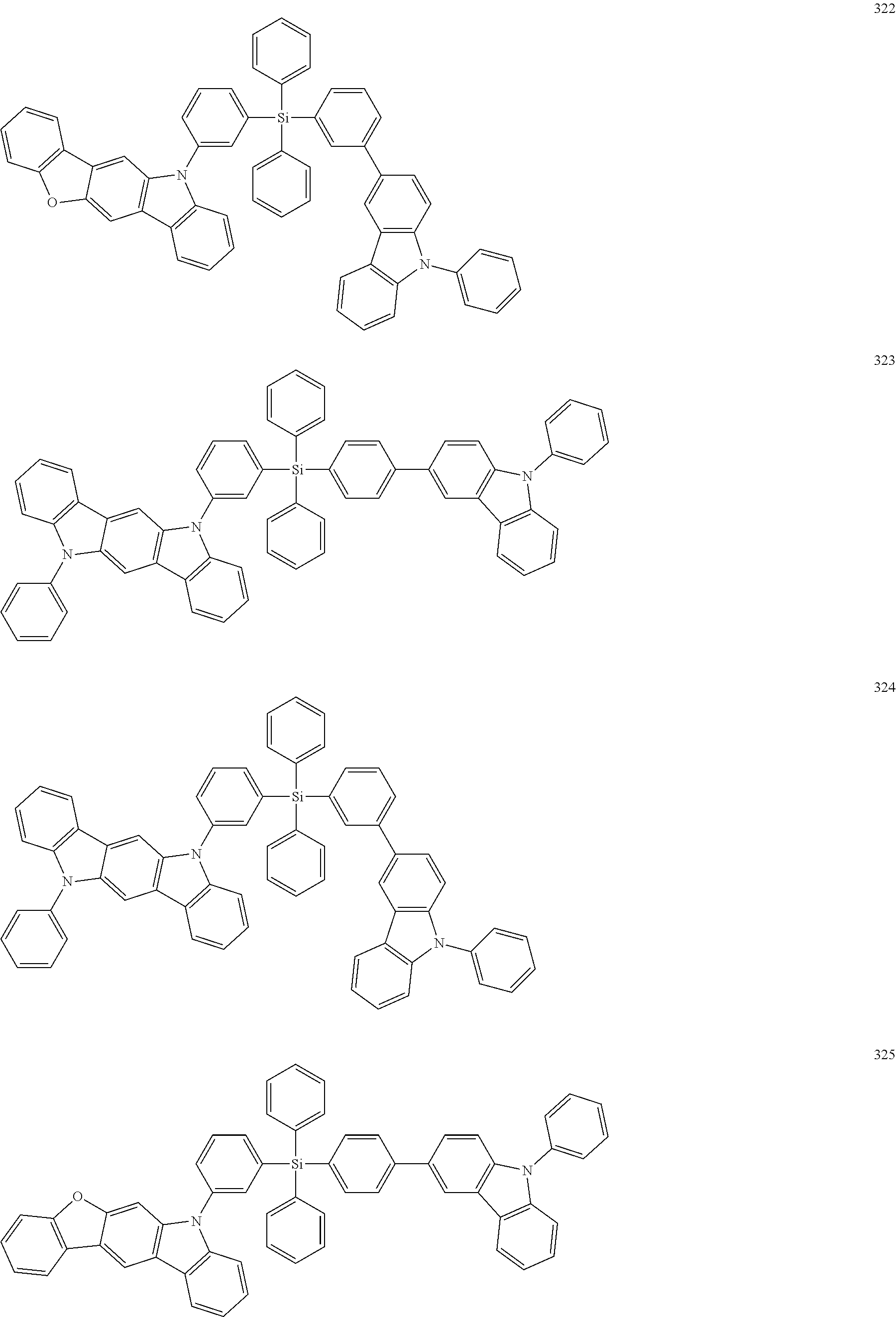

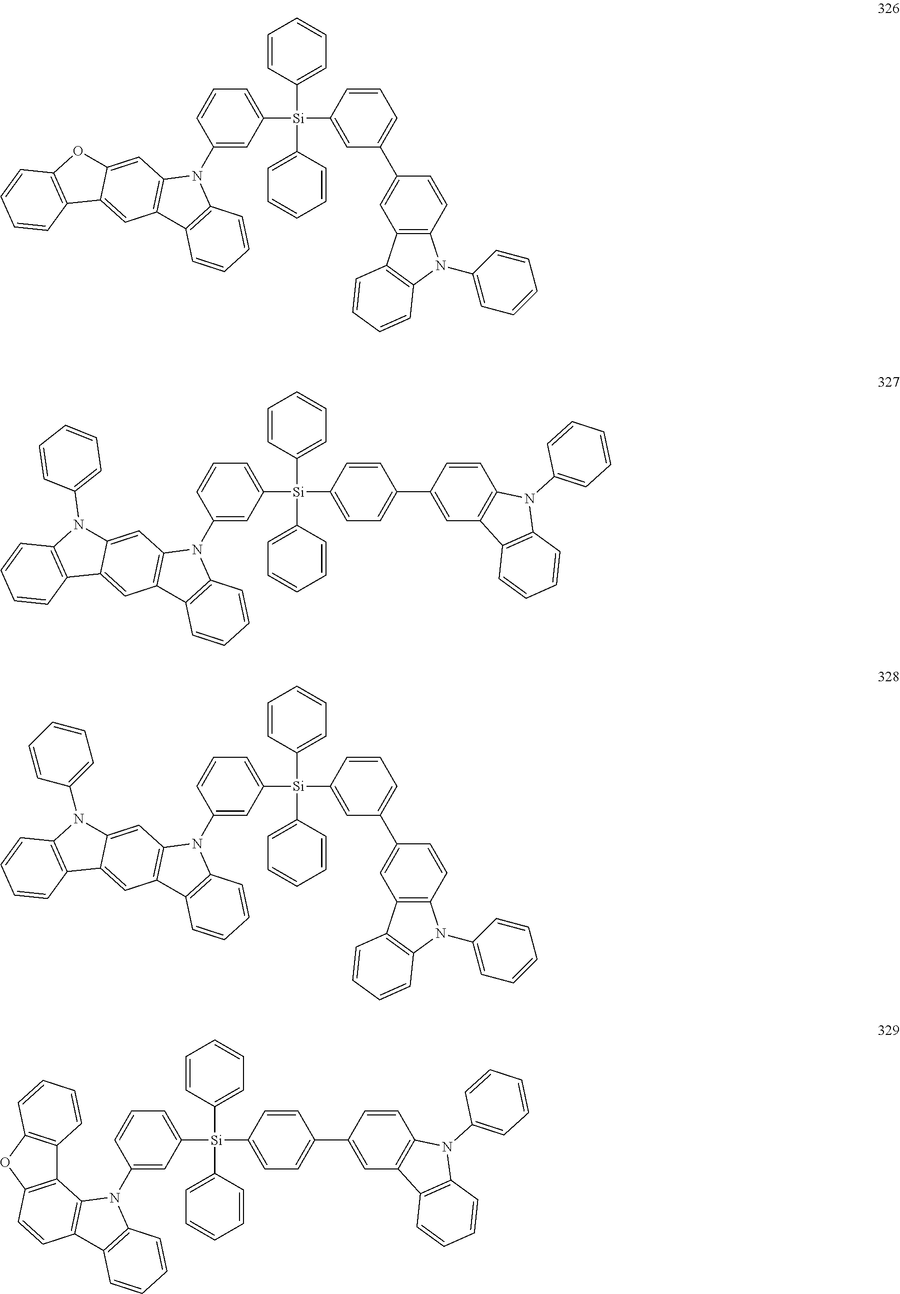

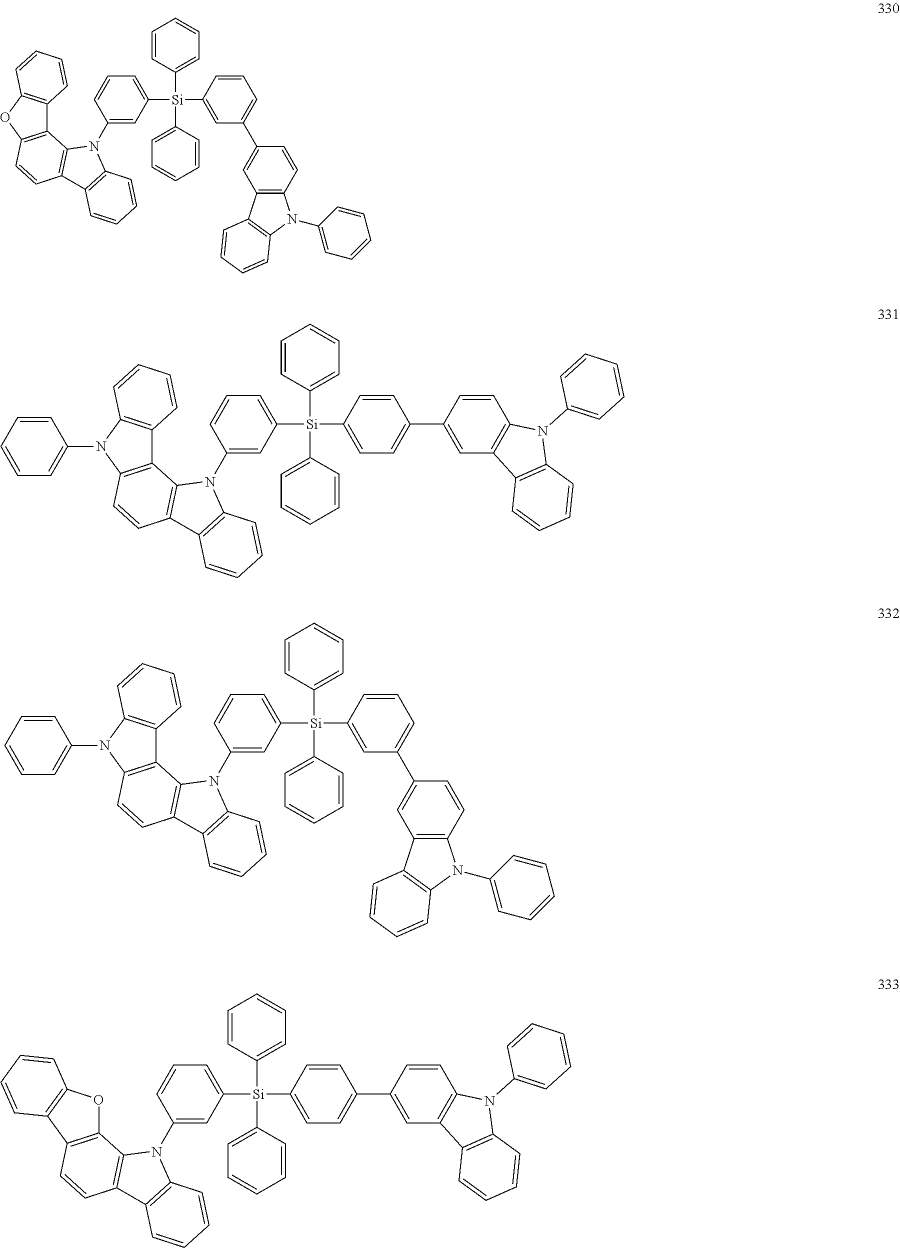

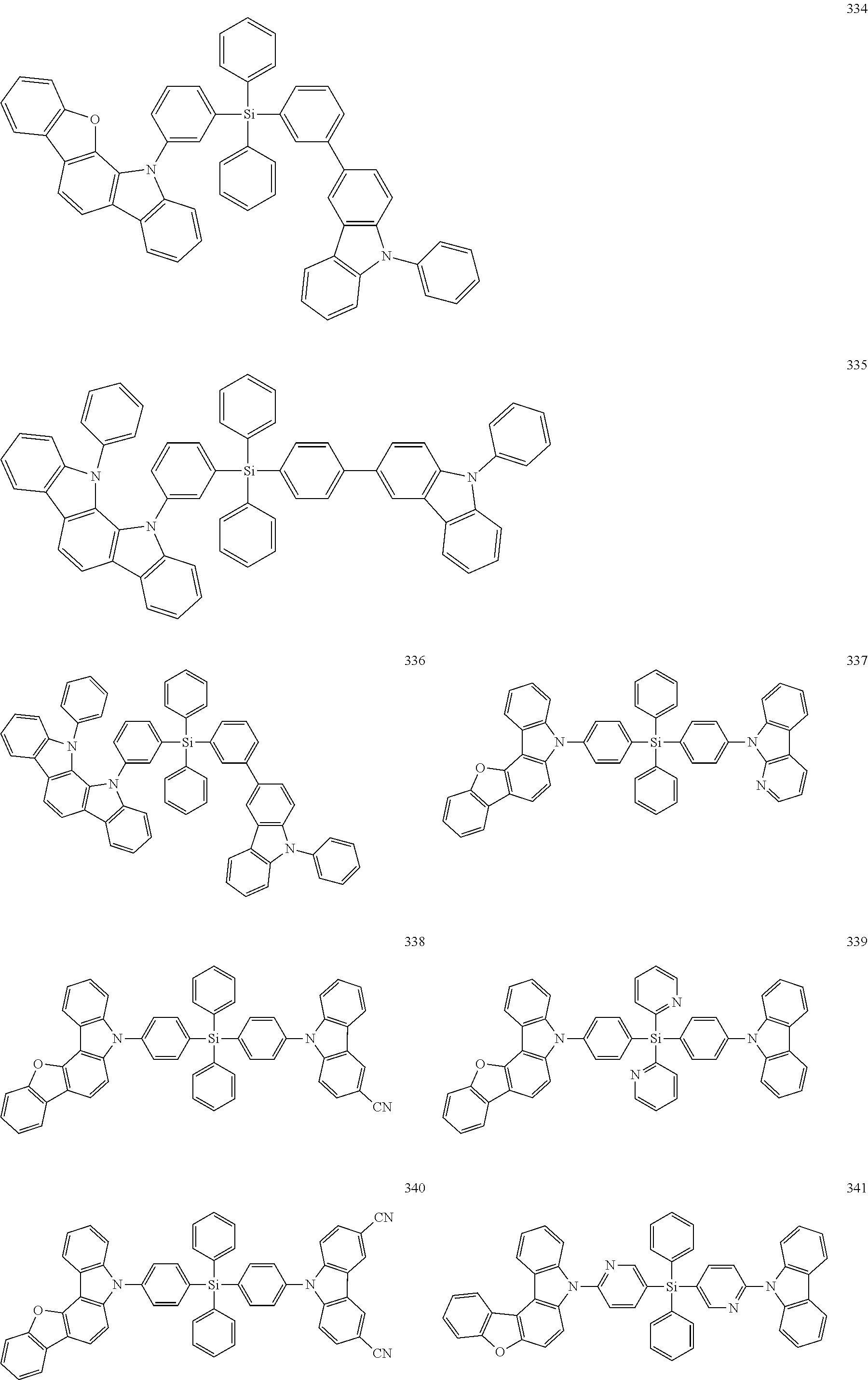

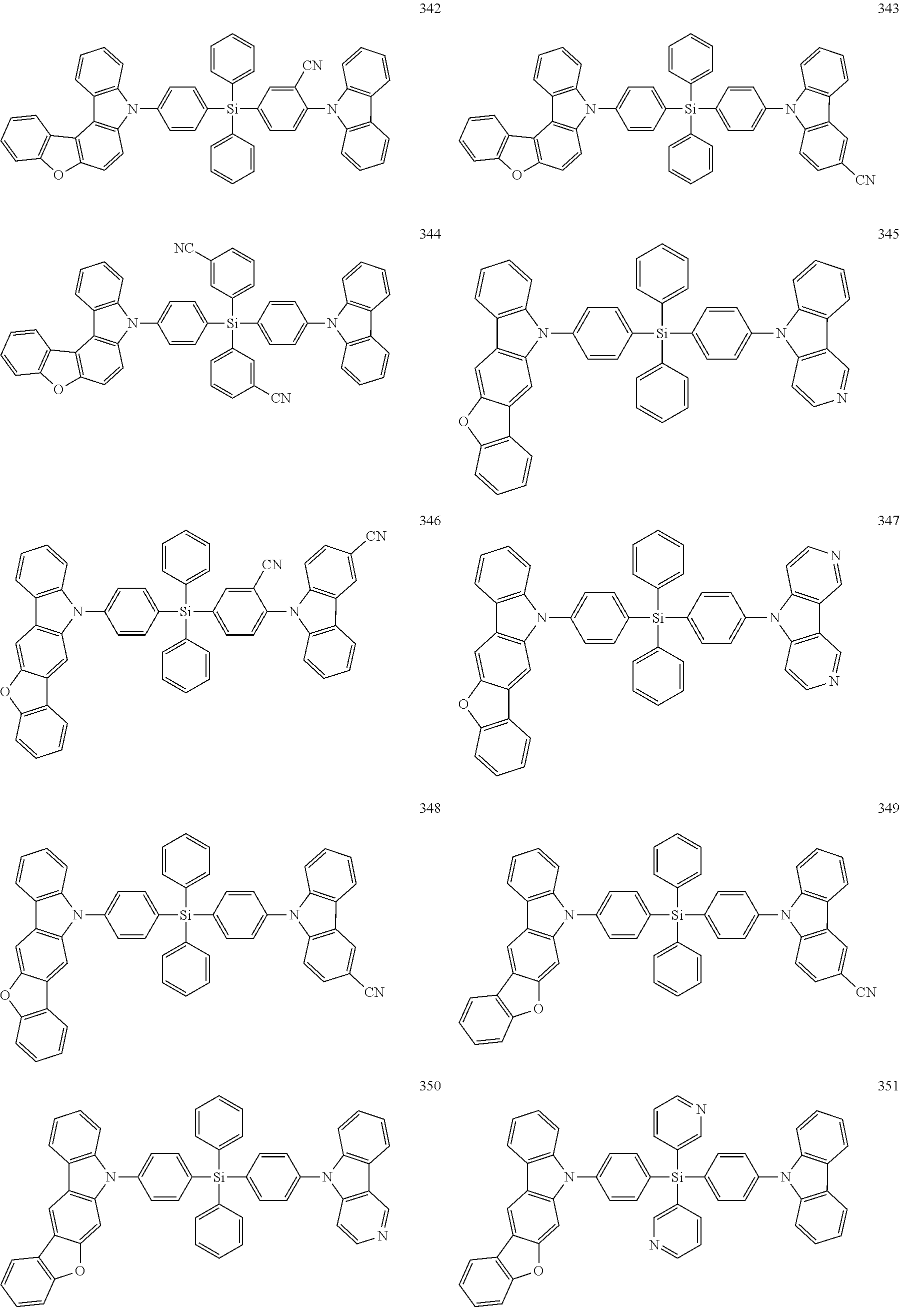

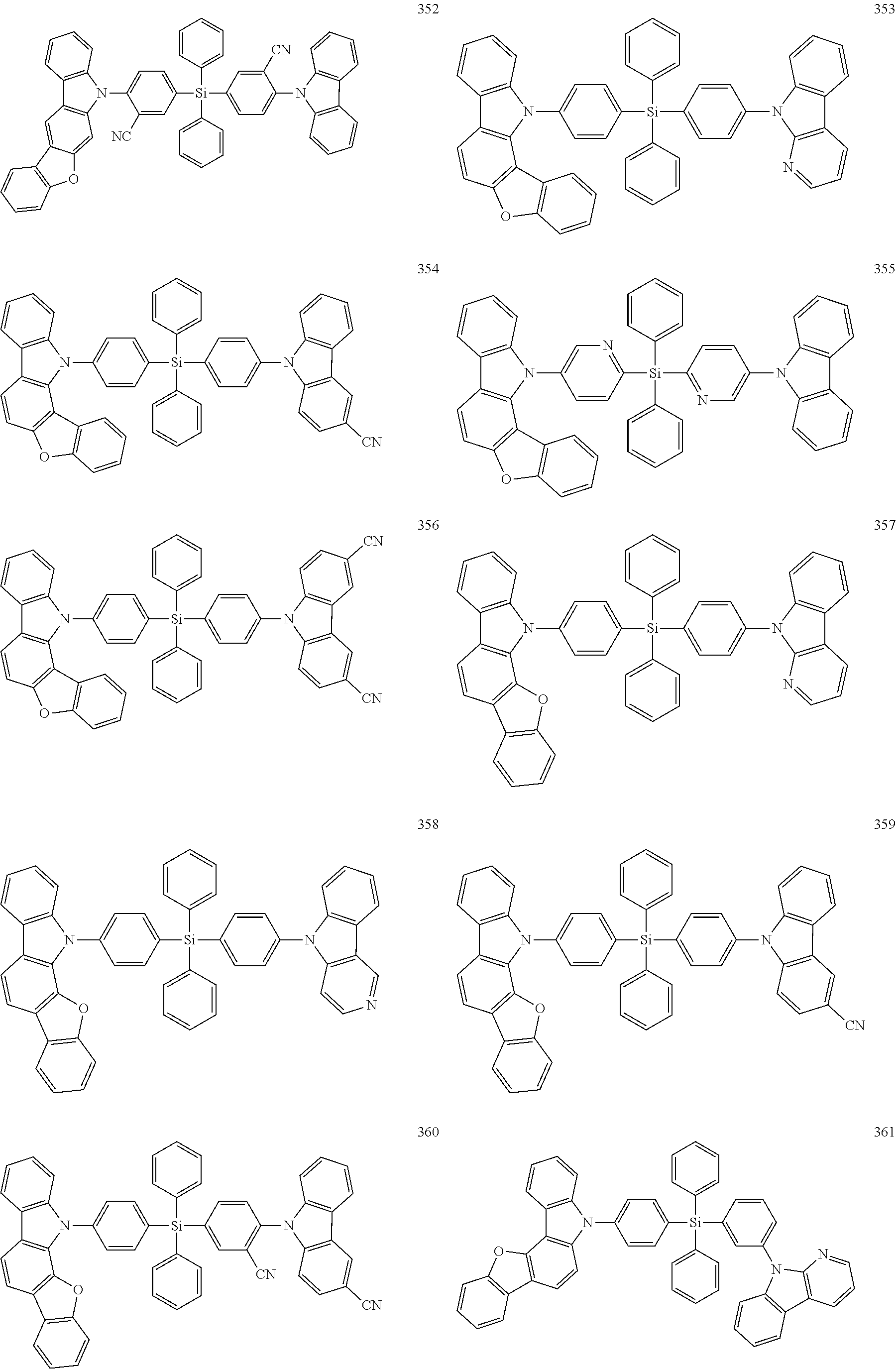

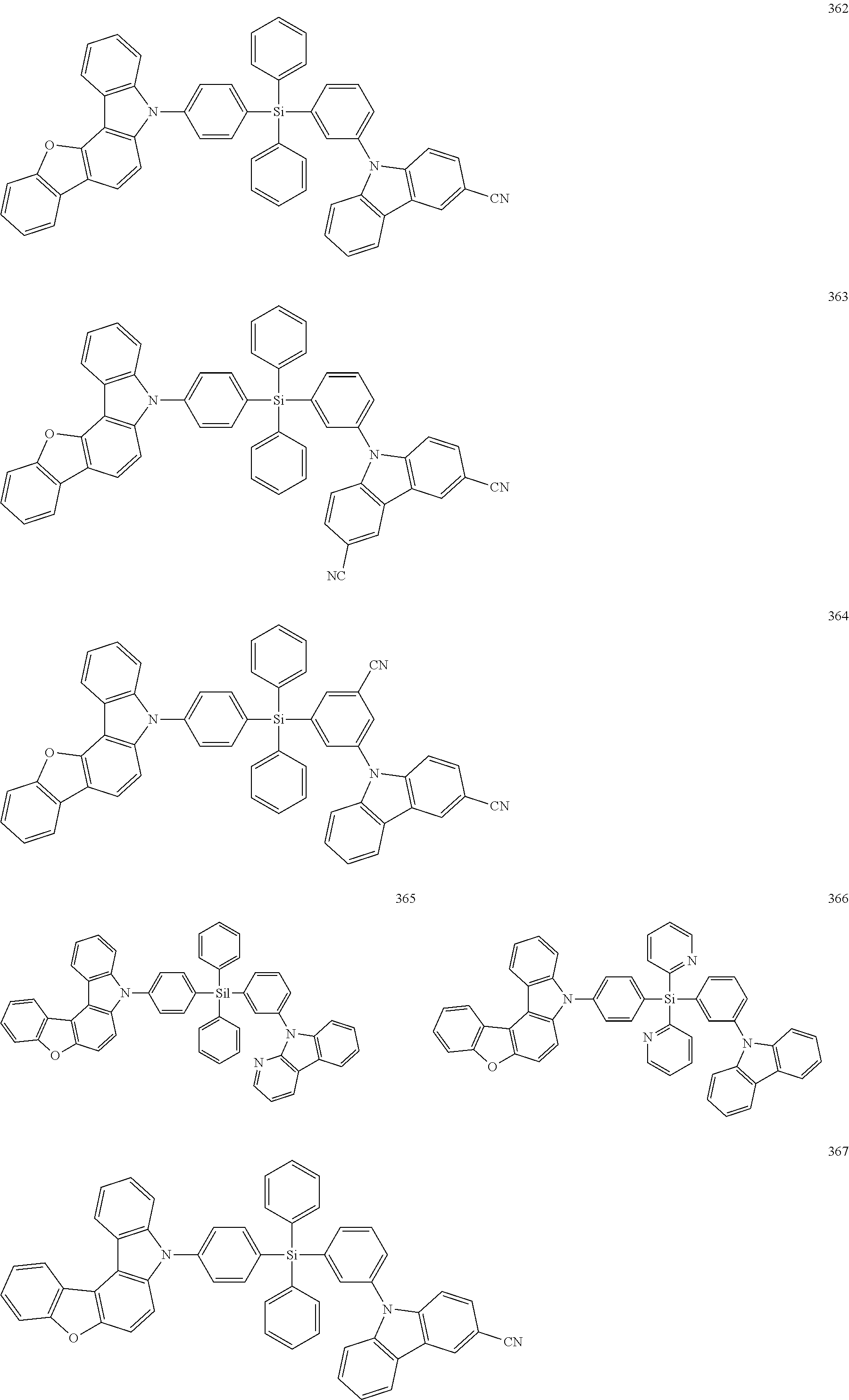

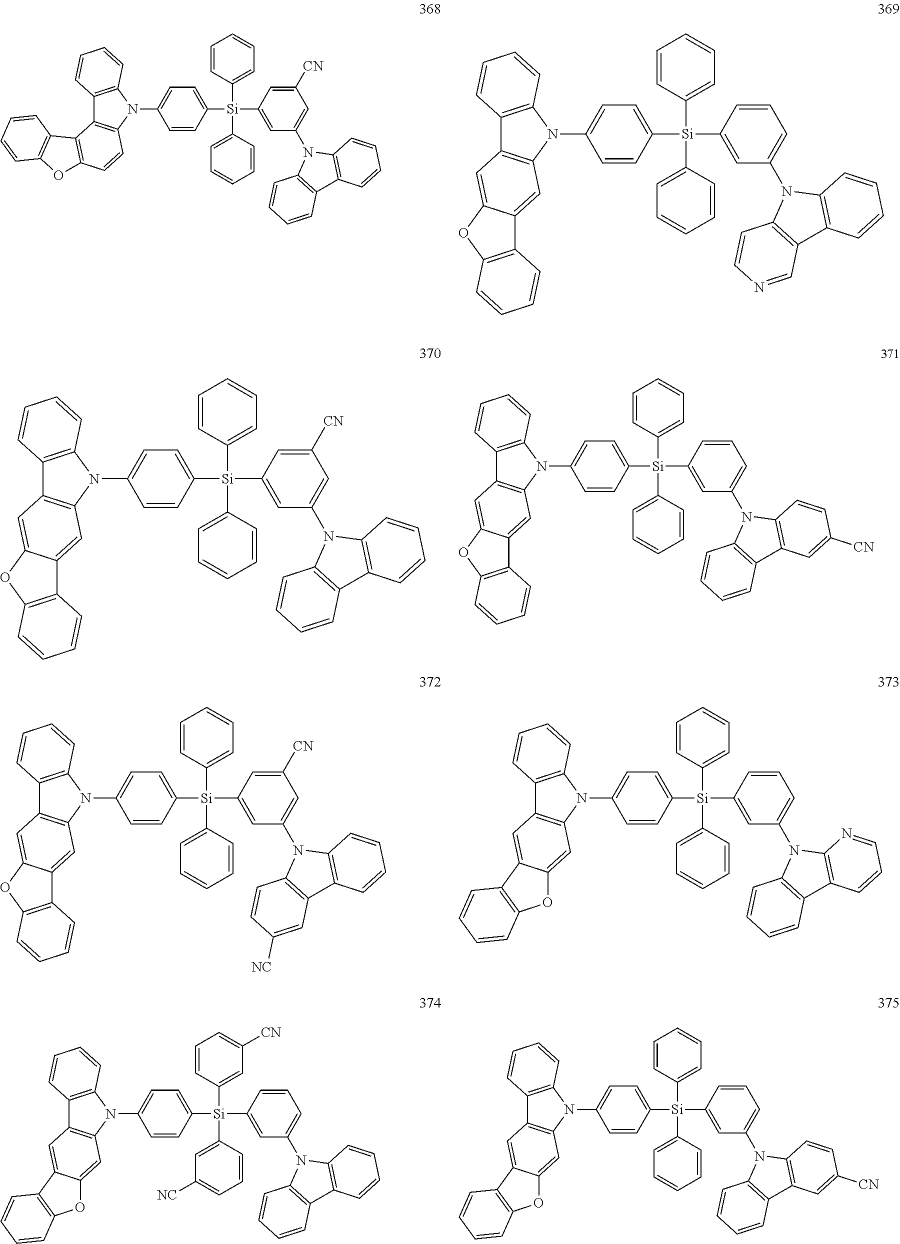

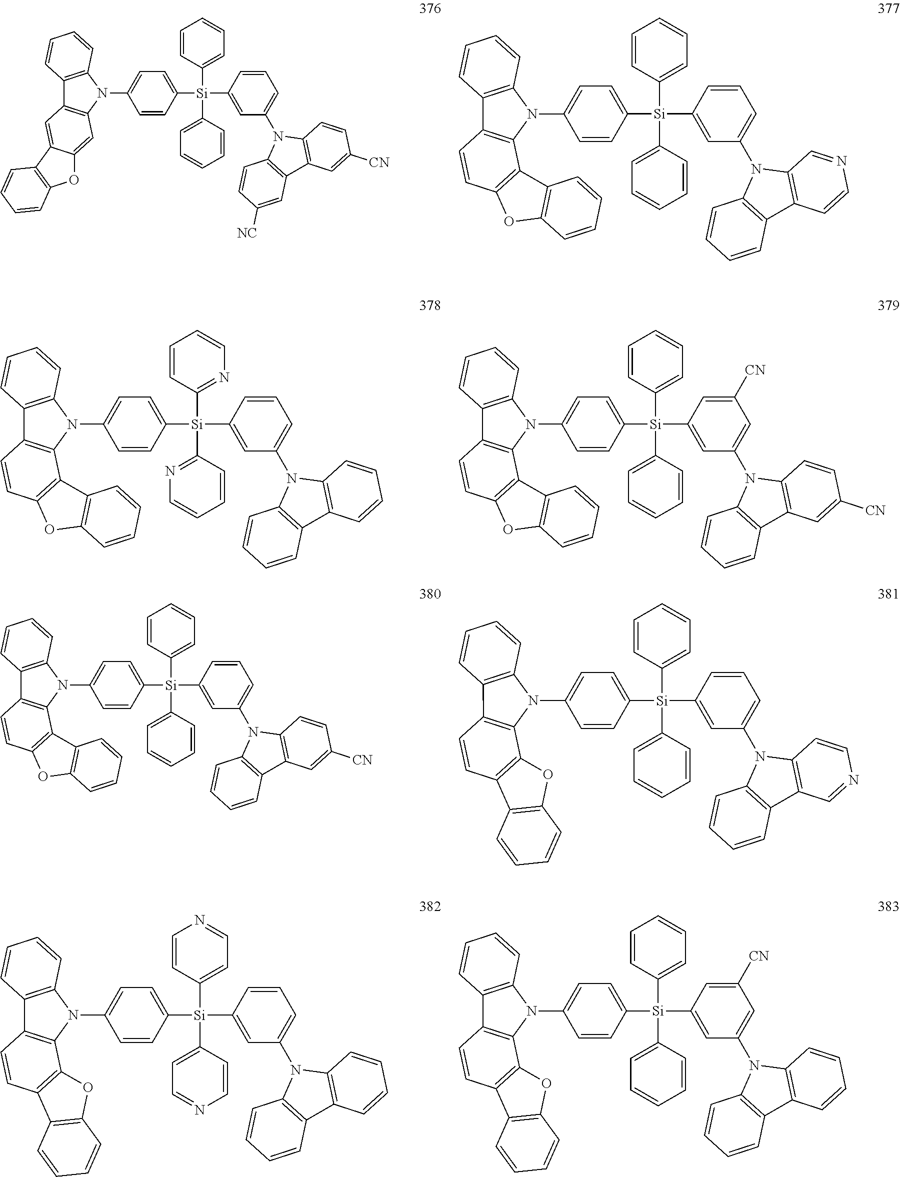

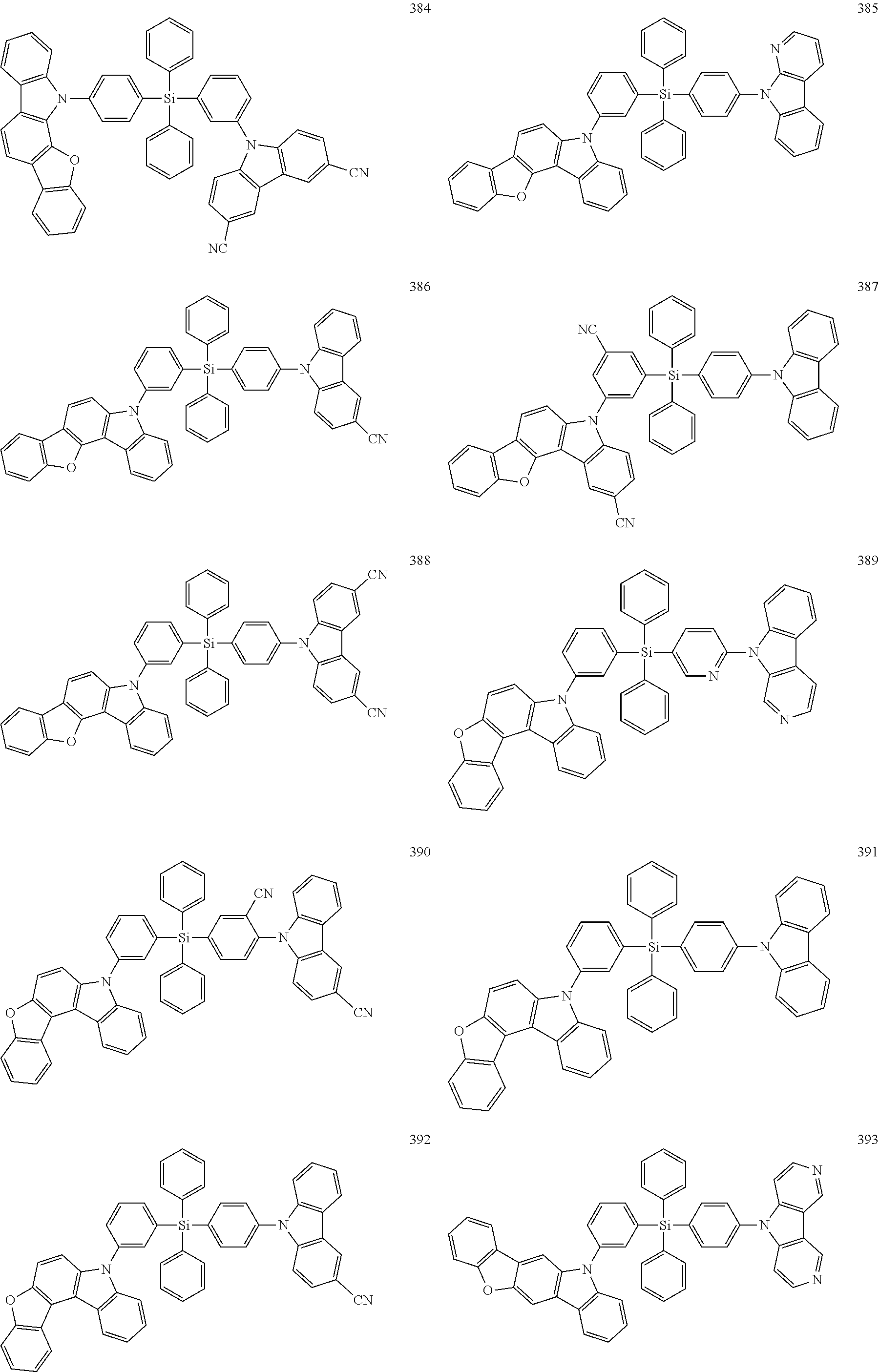

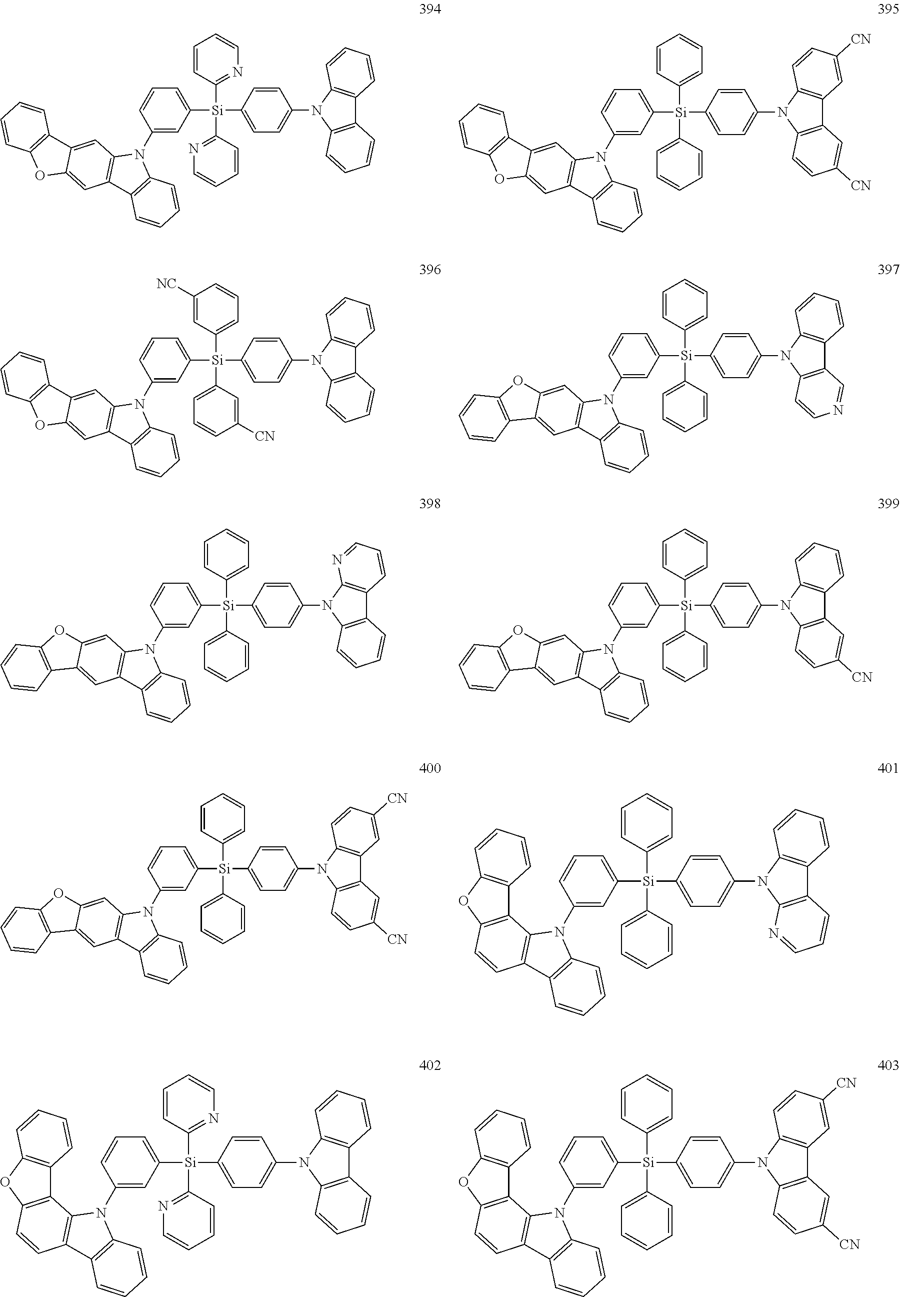

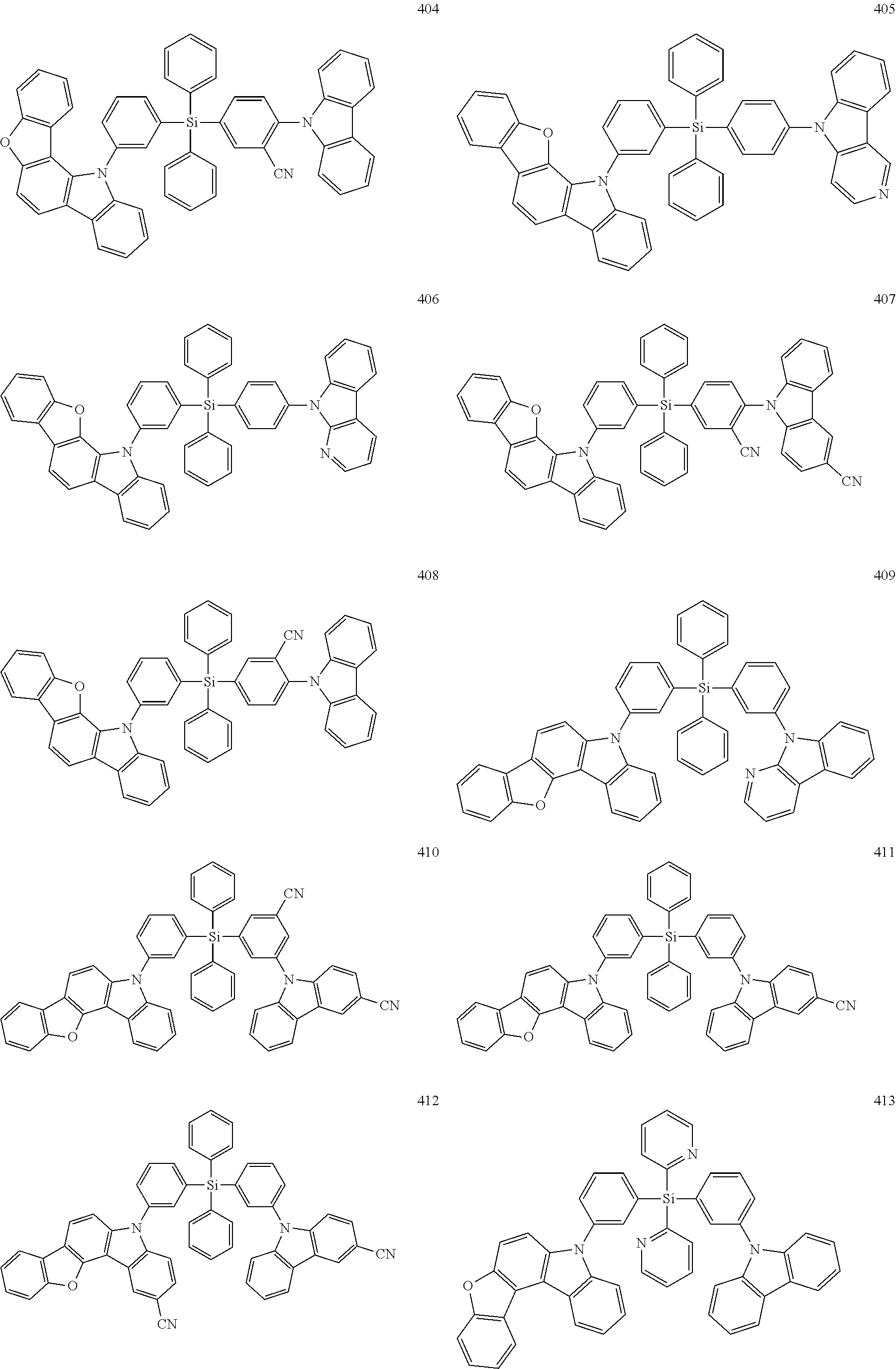

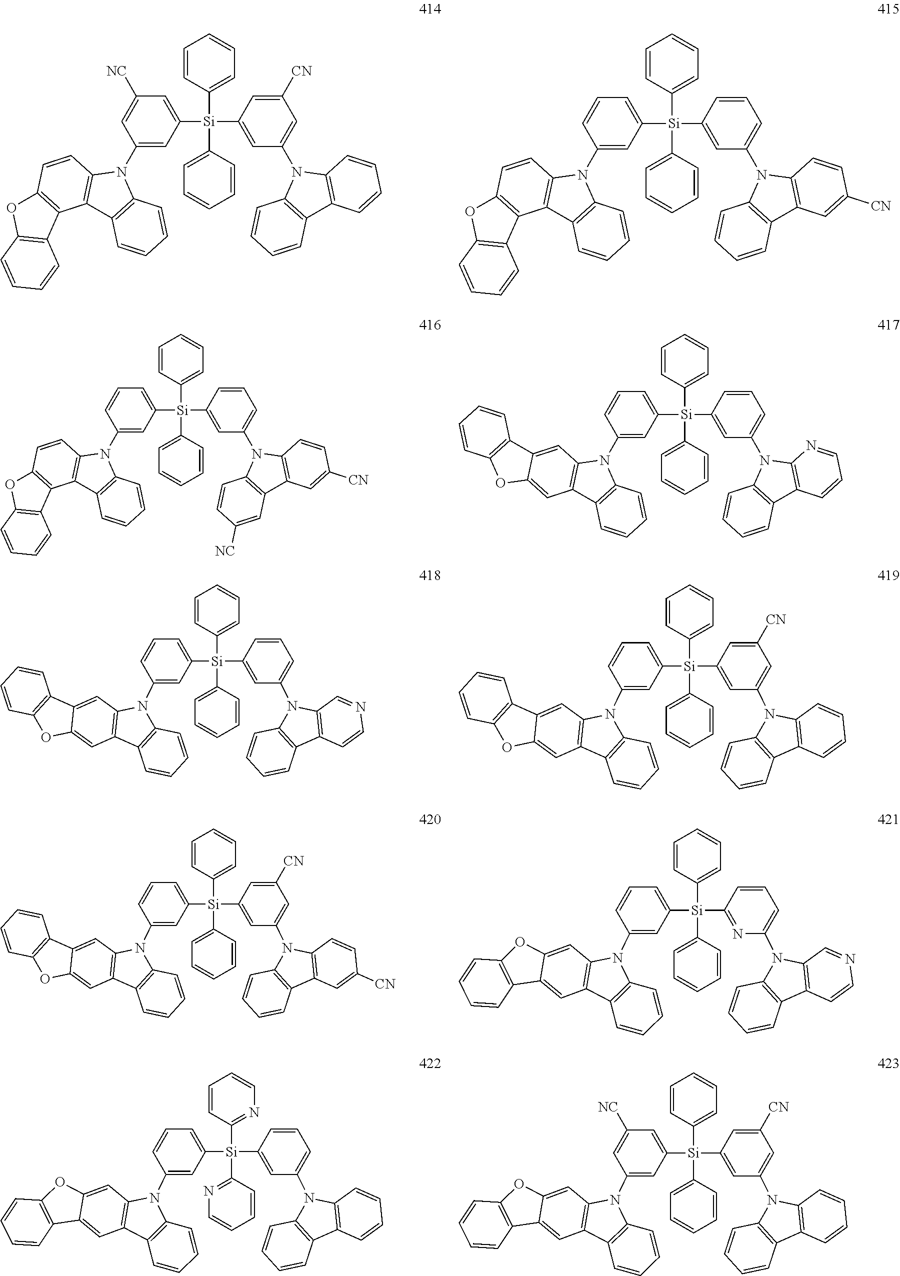

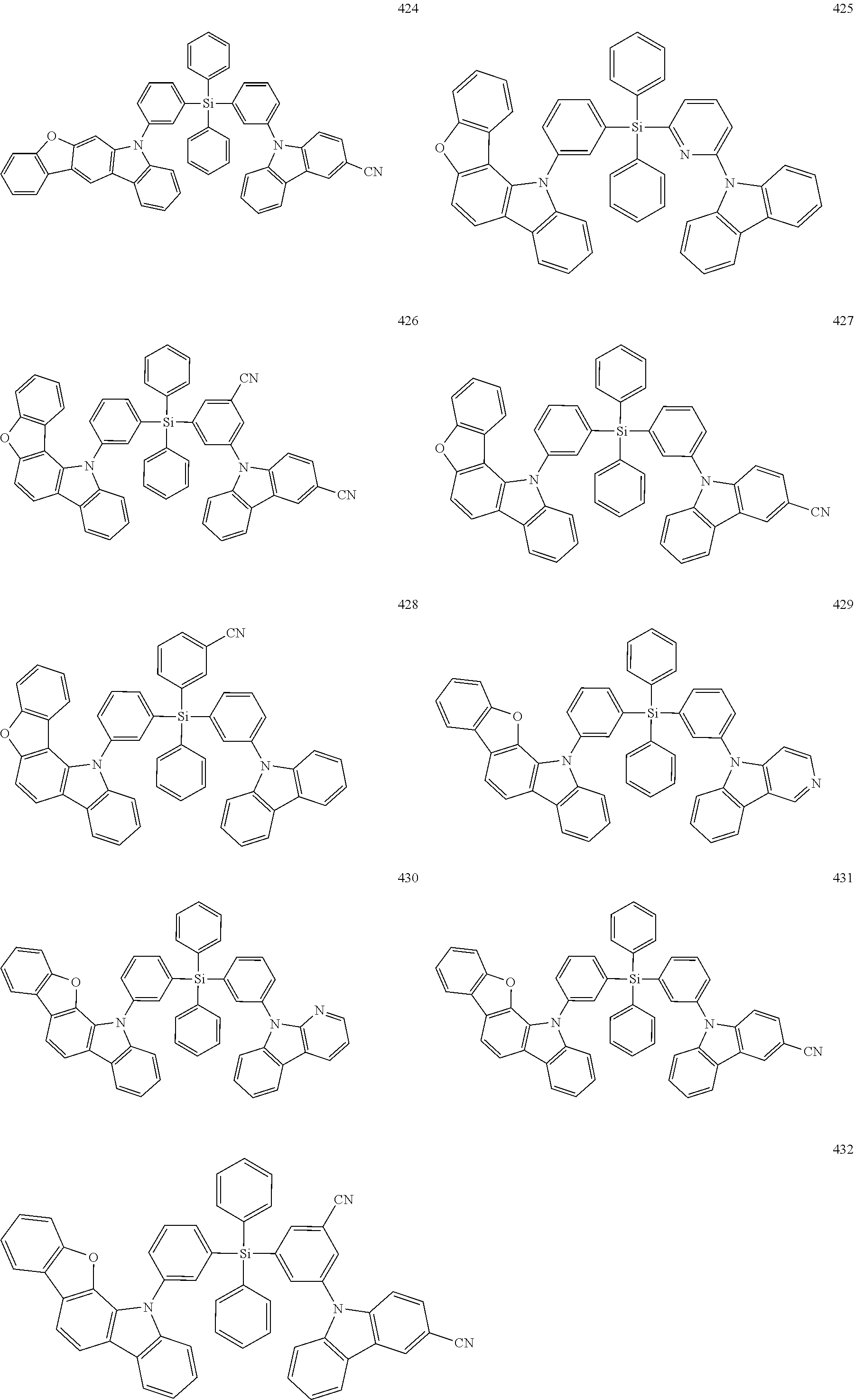

wherein, in Compounds 1 to 432,
“Ph” represents a phenyl group.

In one or more embodiments, the hole-transporting host may include o-CBP or mCP:

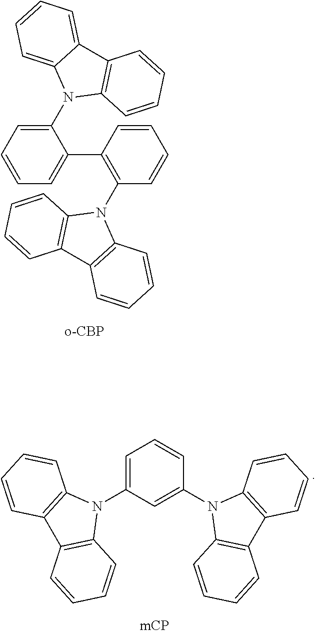 [SMILES: c1ccc(-n2c3ccccc3c3ccccc32)c(-c2ccccc2-n2c3ccccc3c3ccccc32)c1 ; c1cc(-n2c3ccccc3c3ccccc32)cc(-n2c3ccccc3c3ccccc32)c1]

In an embodiment, the host may be a fluorescent host, and the fluorescent host may be represented by, for example, one of Formulae FH-1 to FH-4.

In an embodiment, the fluorescent host may be represented by Formula FH-1:

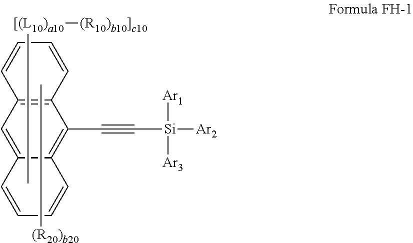

wherein, in Formula FH-1,
Ar₁ to Ar₃ may each independently be a substituted or unsubstituted C₁-C₆₀ alkyl group, a substituted or unsubstituted C₂-C₆₀ alkenyl group, a substituted or unsubstituted C₂-C₆₀ alkynyl group, a substituted or unsubstituted C₁-C₆₀ alkoxy group, a substituted or unsubstituted C₃-C₁₀ cycloalkyl group, a substituted or unsubstituted C₁-C₁₀ heterocycloalkyl group, a substituted or unsubstituted C₃-C₁₀ cycloalkenyl group, a substituted or unsubstituted C₂-C₁₀ heterocycloalkenyl group, a substituted or unsubstituted C₆-C₆₀ aryl group, a substituted or unsubstituted C₆-C₆₀ aryloxy group, a substituted or unsubstituted C₆-C₆₀ arylthio group, a substituted or unsubstituted C₁-C₆₀ heteroaryl group, a substituted or unsubstituted C₁-C₆₀ hetero aryloxy group, a substituted or unsubstituted C₁-C₆₀ hetero arylthio group, a substituted or unsubstituted monovalent non-aromatic condensed polycyclic group, a substituted or unsubstituted monovalent non-aromatic condensed heteropolycyclic group, —Si(Q₁)(Q₂)(Q₃), —N(Q₁)(Q₂), —B(Q₁)(Q₂), —C(═O)(Q₁), —S(═O)₂(Q₁), or —P(═O)(Q₁)(Q₂),
at least one of Ar₁ to Ar₃ may each independently be a substituted or unsubstituted C₆-C₆₀ aryl group, a substituted or unsubstituted C₁-C₆₀ heteroaryl group, a substituted or unsubstituted monovalent non-aromatic condensed polycyclic group, or a substituted or unsubstituted monovalent non-aromatic condensed heteropolycyclic group,
L₁₀ may be an unsubstituted or substituted C₅-C₃₀ carbocyclic group or an unsubstituted or substituted C₁-C₃₀ heterocyclic group,
a10 may be an integer from 1 to 3, wherein, when al is 2 or greater, two or more of L₁ may be identical to or different from each other,
R₁₀ and R₂₀ may each independently be hydrogen, deuterium, —F, —Cl, —Br, —I, —SF₅, a hydroxyl group, a cyano group, a nitro group, an amidino group, a hydrazine group, a hydrazone group, a carboxylic acid group or a salt thereof, a sulfonic acid group or a salt thereof, a phosphoric acid group or a salt thereof, a substituted or unsubstituted C₁-C₆₀ alkyl group, a substituted or unsubstituted C₂-C₆₀ alkenyl group, a substituted or unsubstituted C₂-C₆₀ alkynyl group, a substituted or unsubstituted C₁-C₆₀ alkoxy group, a substituted or unsubstituted C₃-C₁₀ cycloalkyl group, a substituted or unsubstituted C₁-C₁₀ heterocycloalkyl group, a substituted or unsubstituted C₃-C₁₀ cycloalkenyl group, a substituted or unsubstituted C₂-C₁₀ heterocycloalkenyl group, a substituted or unsubstituted C₆-C₆₀ aryl group, a substituted or unsubstituted C₆-C₆₀ aryloxy group, a substituted or unsubstituted C₆-C₆₀ arylthio group, a substituted or unsubstituted C₁-C₆₀ heteroaryl group, a substituted or unsubstituted monovalent non-aromatic condensed polycyclic group, a substituted or unsubstituted monovalent non-aromatic condensed heteropolycyclic group, —N(Q₁)(Q₂), —Si(Q₃)(Q₄)(Q₅), —B(Q₆)(Q₇), or —P(═O)(Q₈)(Q₉),
b10 and b20 may each independently an integer from 1 to 8,
wherein, when b10 is 2 or greater, two or more of R₁₀ may be identical to or different from each other, and when b20 is 2 or greater, two or more of R₂₀ may be identical to or different from each other, and
c10 may be an integer from 1 to 9,
wherein, when c10 is 2 or greater, two or more of -[(L₁₀)ₐ₁₀-(R₁₀)_{b10}] may be identical to or different from each other.

In an embodiment, the fluorescent host represented by Formula FH-1 may be a compound of Group FH1, but embodiments of the present disclosure are not limited thereto:

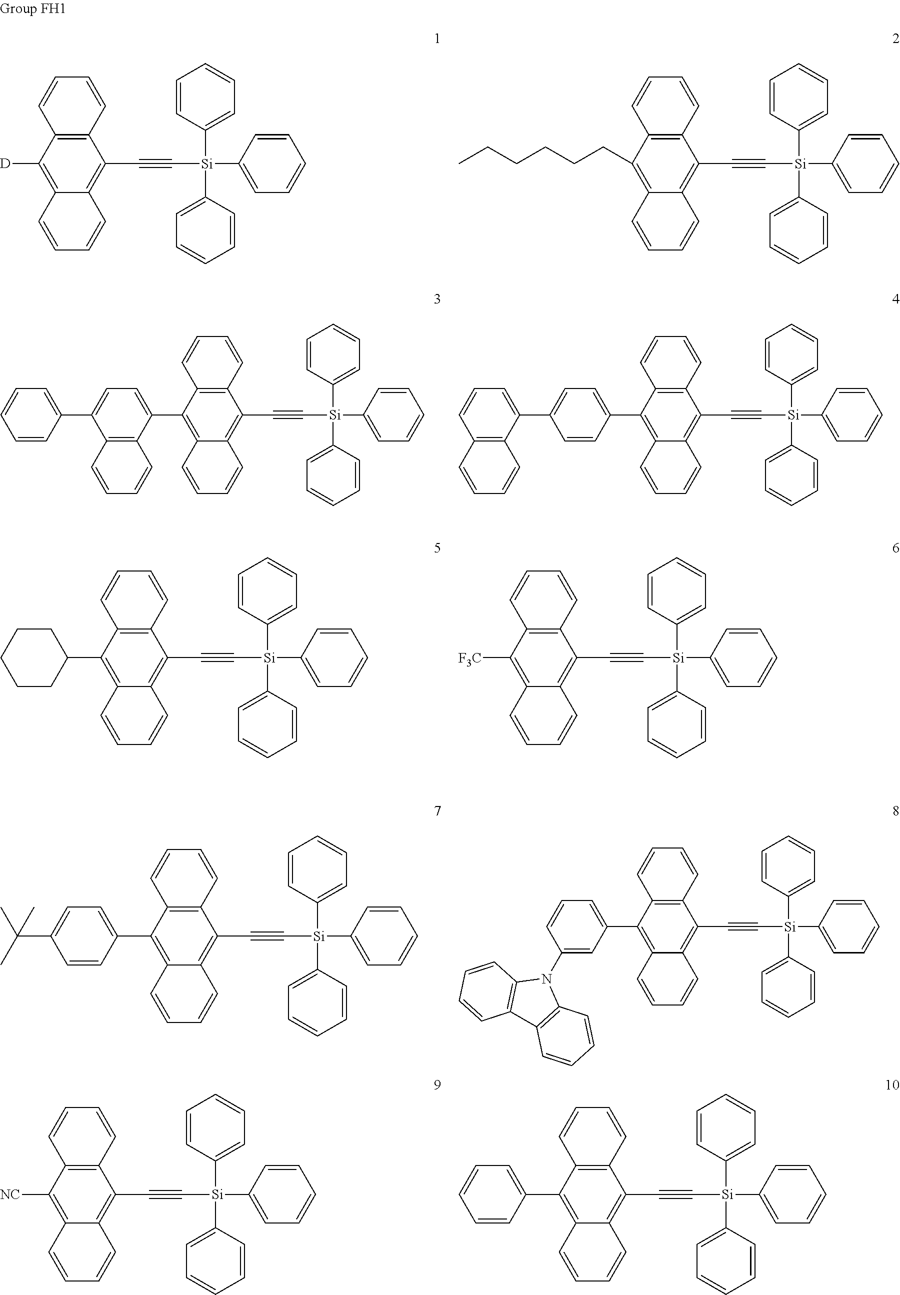

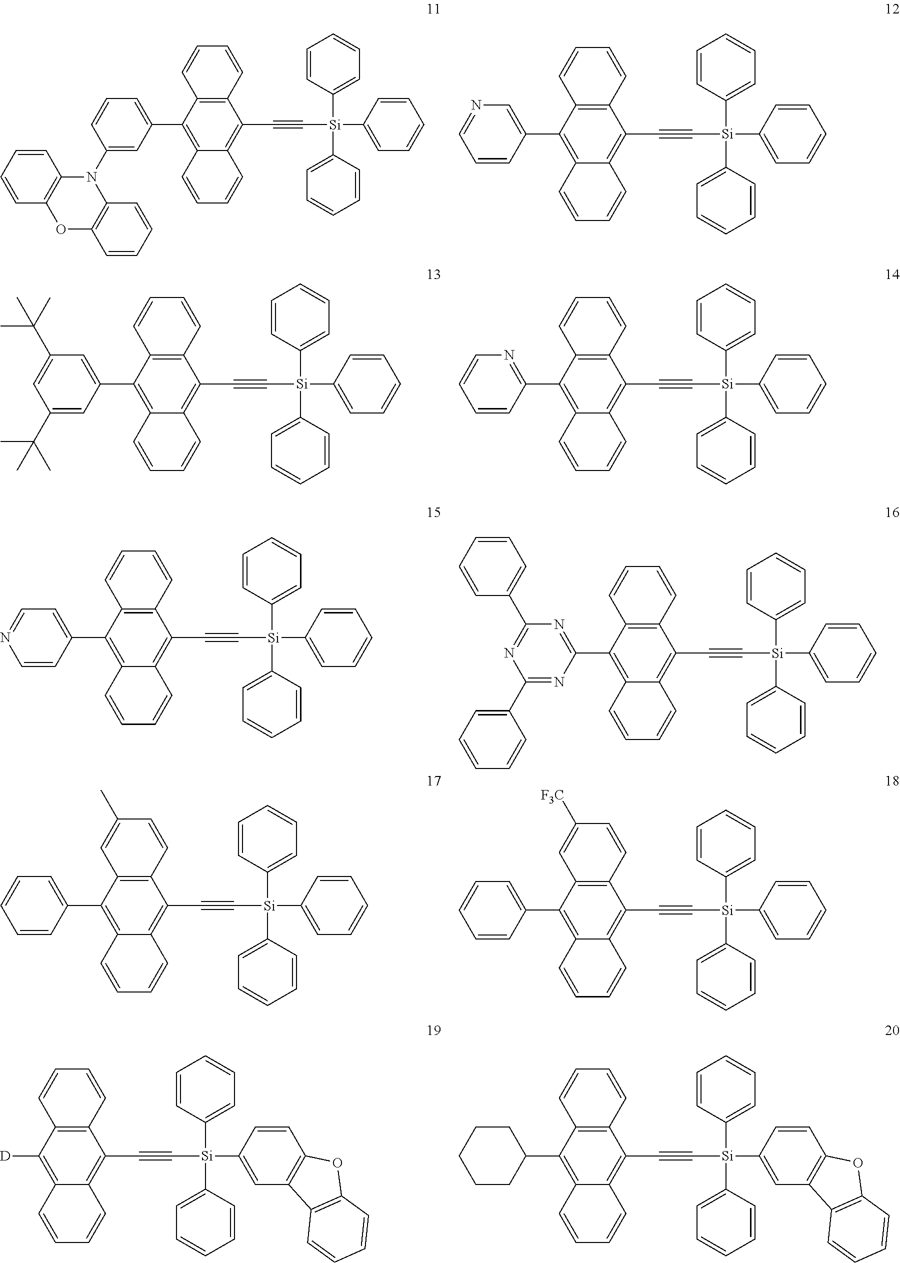

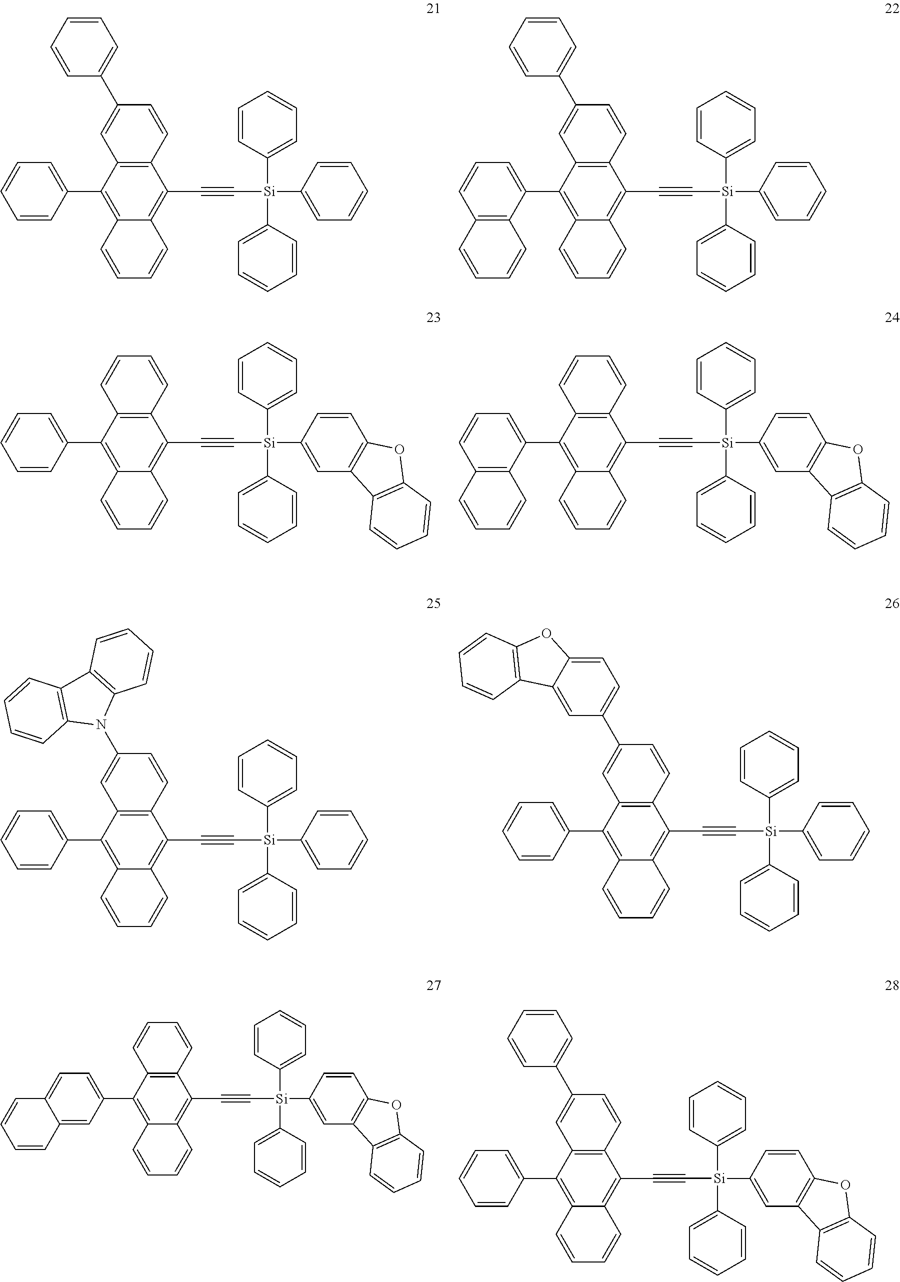

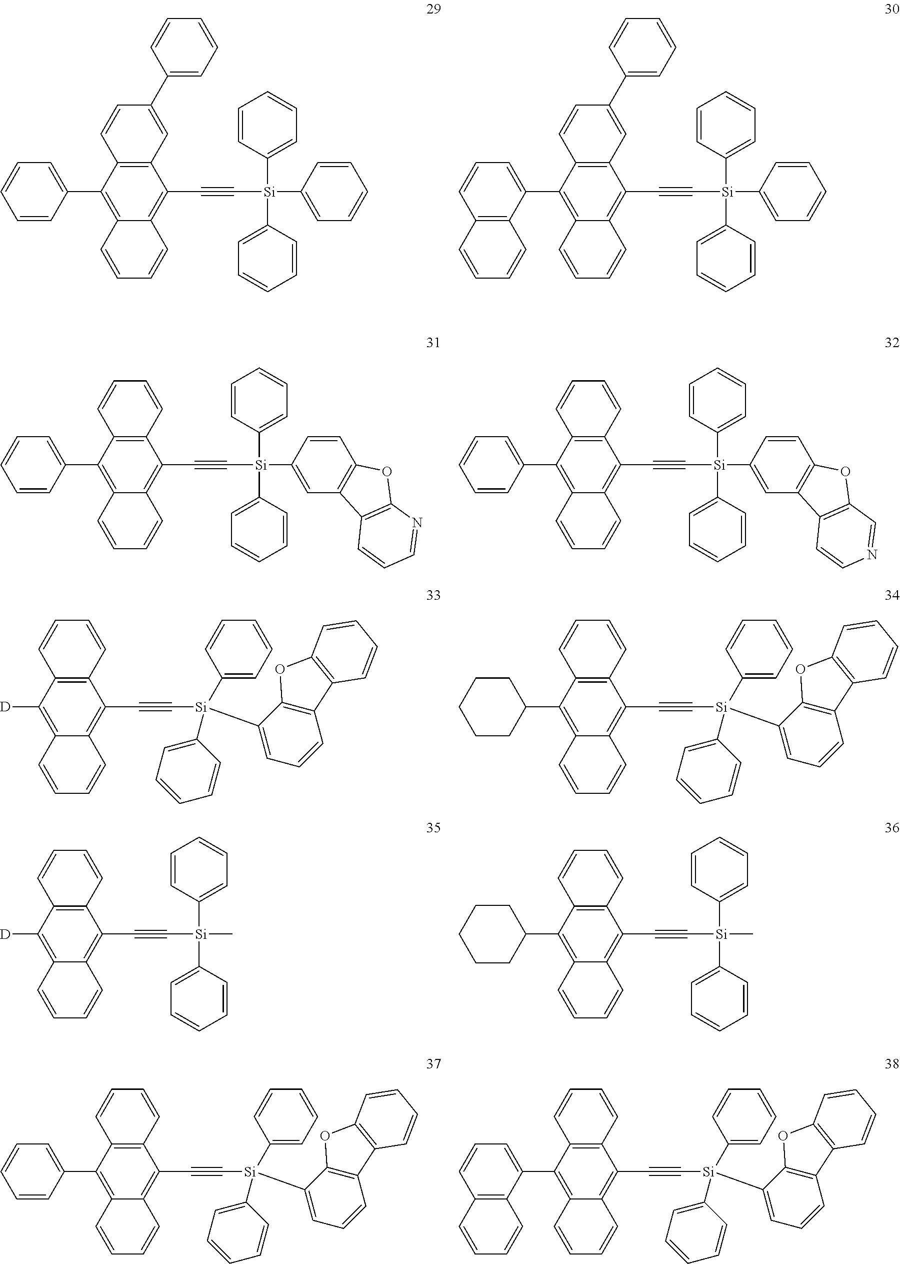

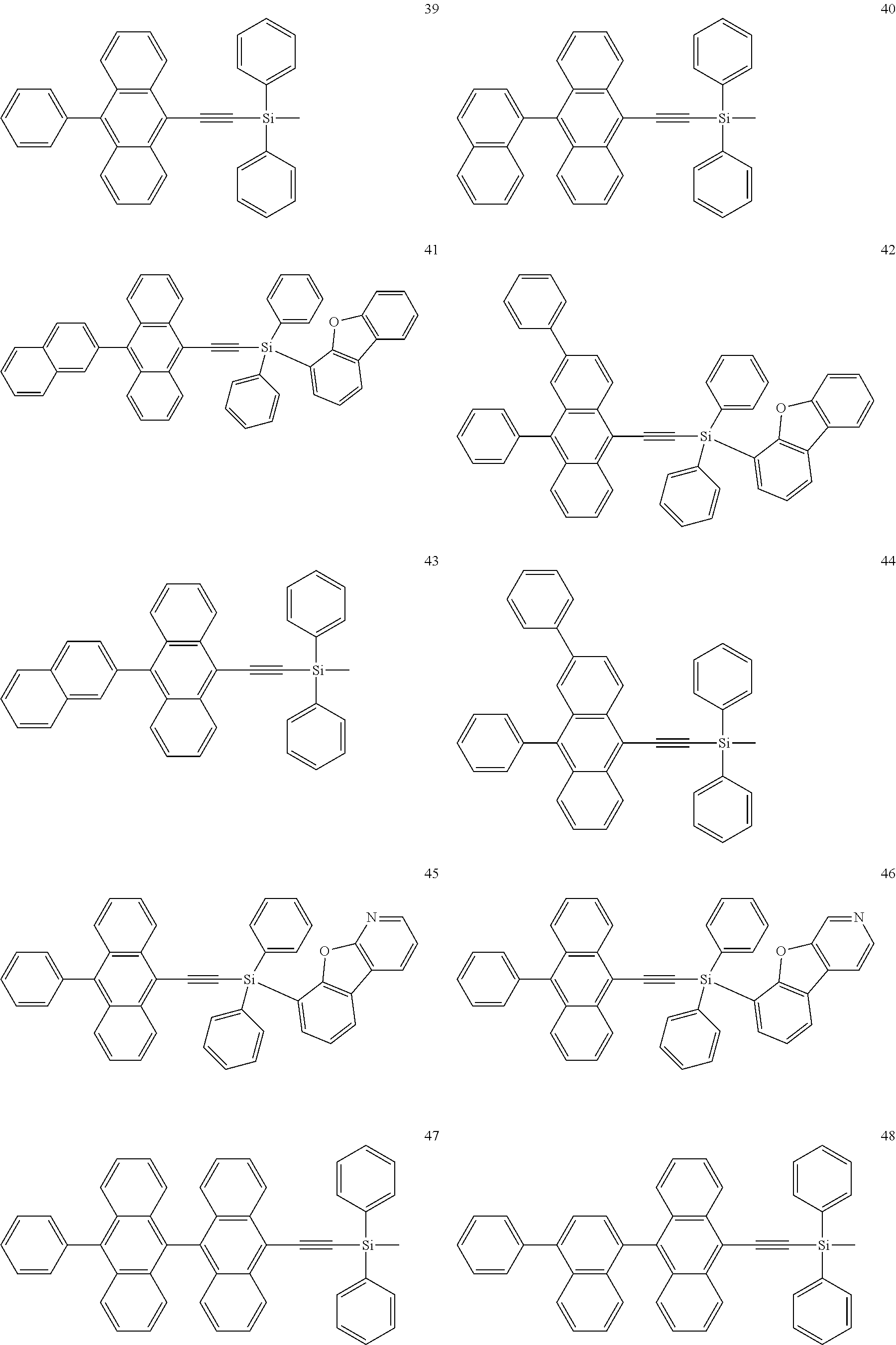

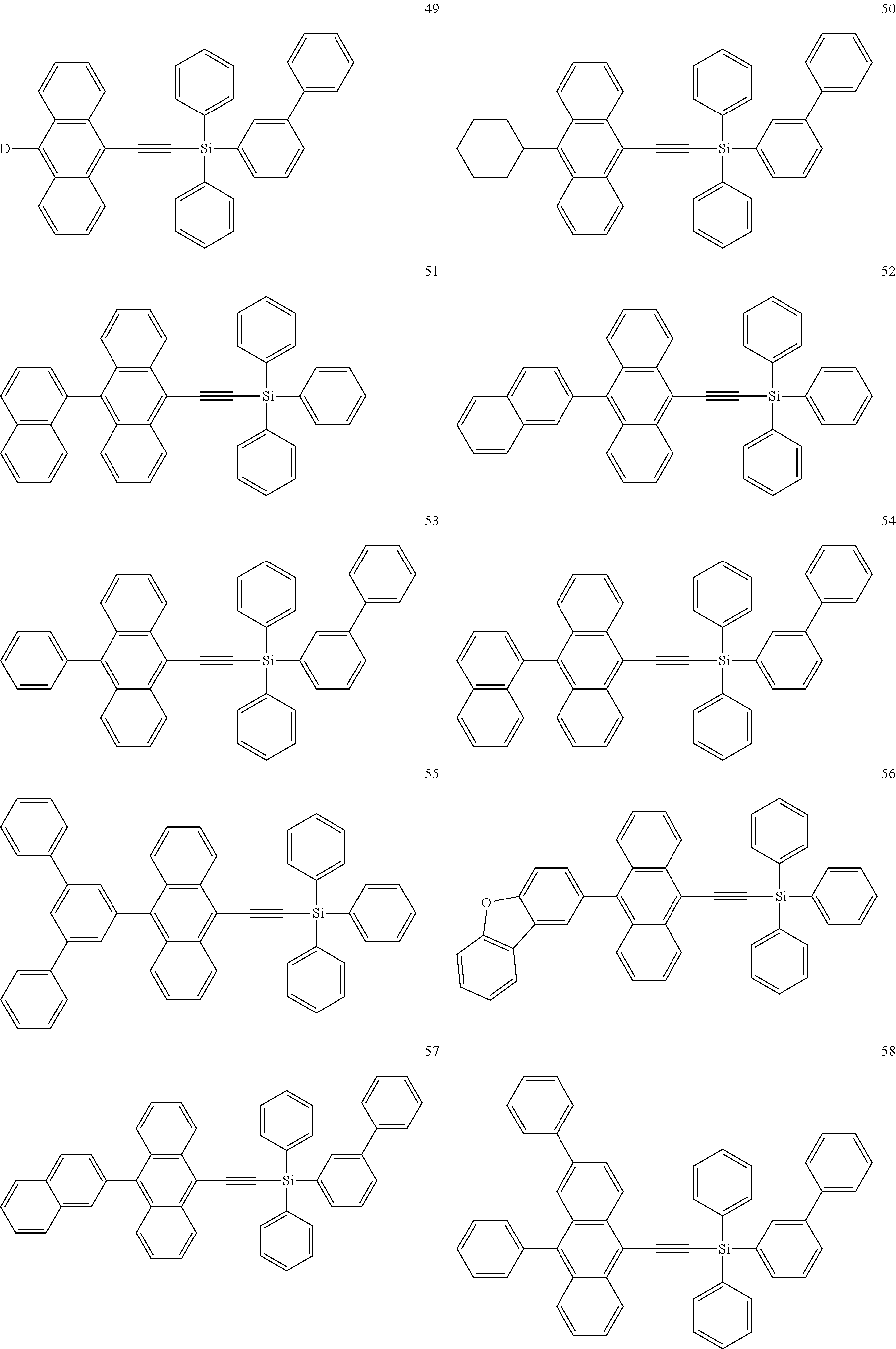

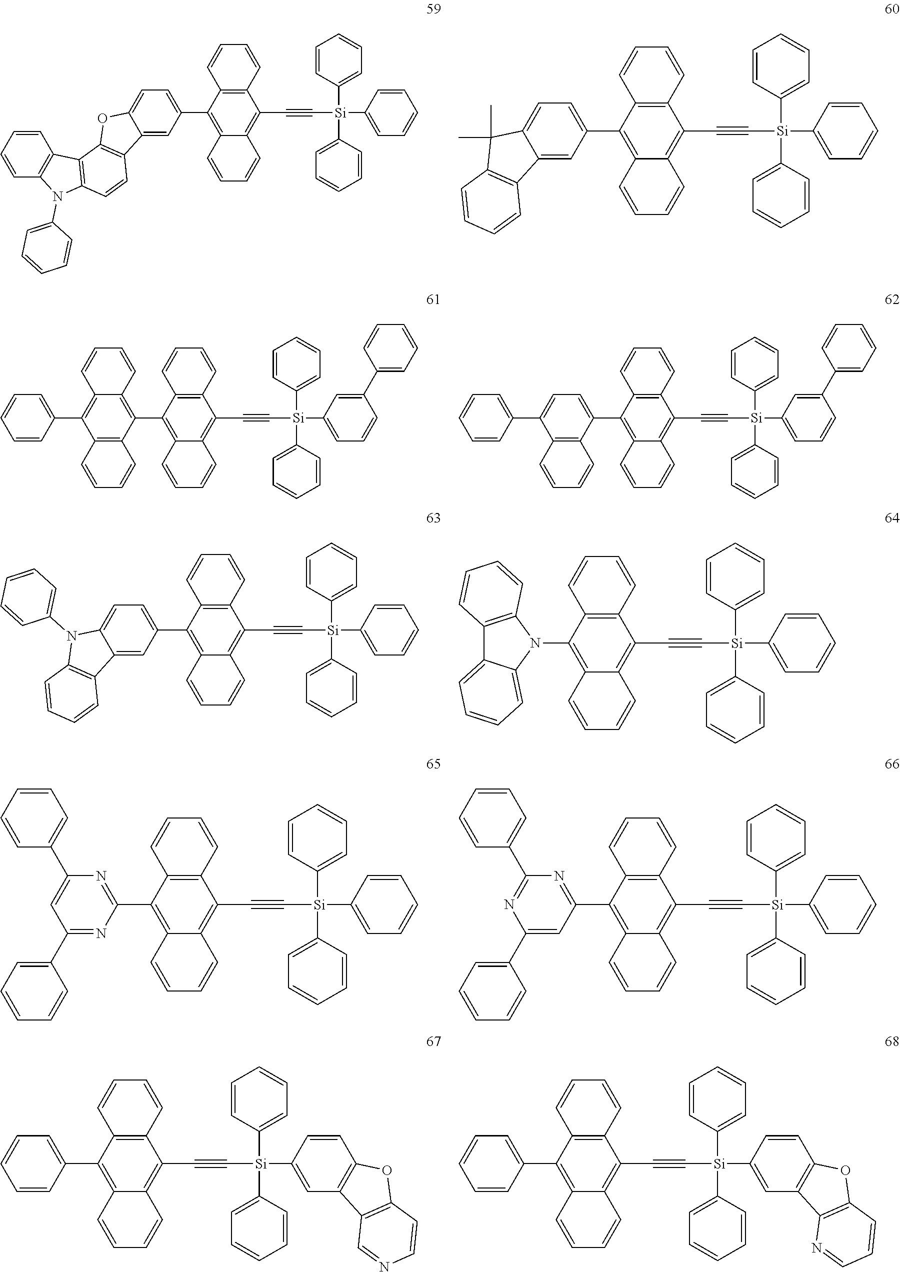

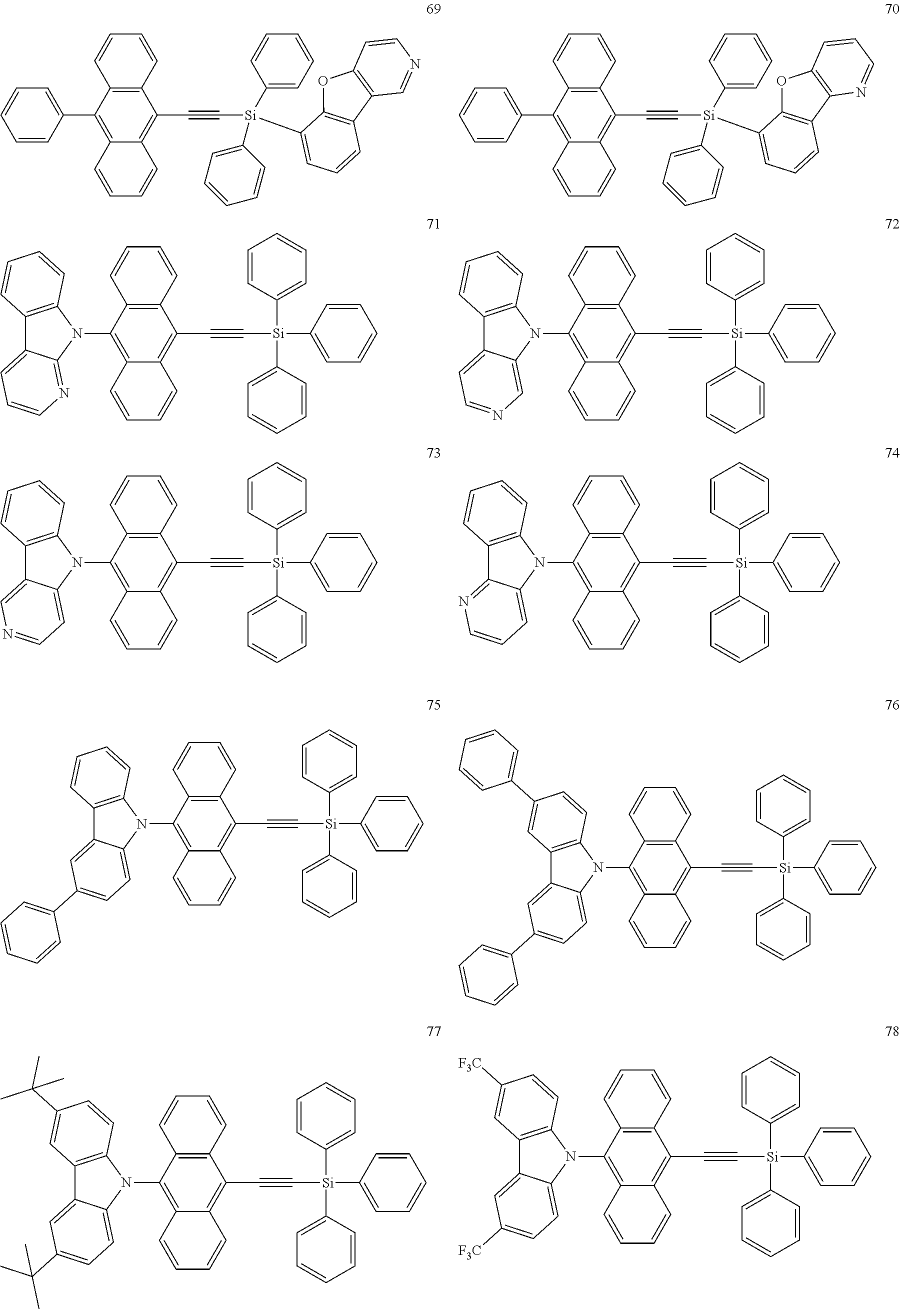

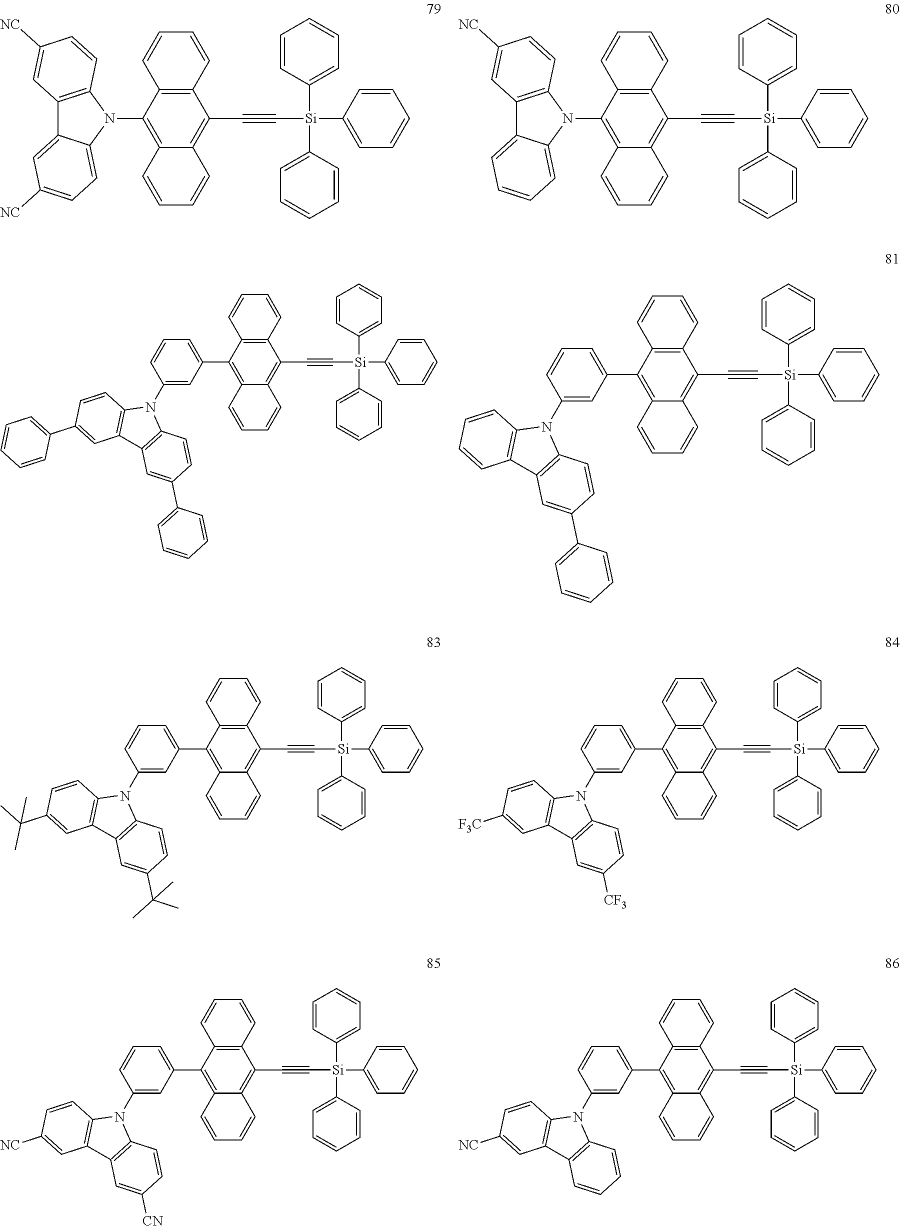

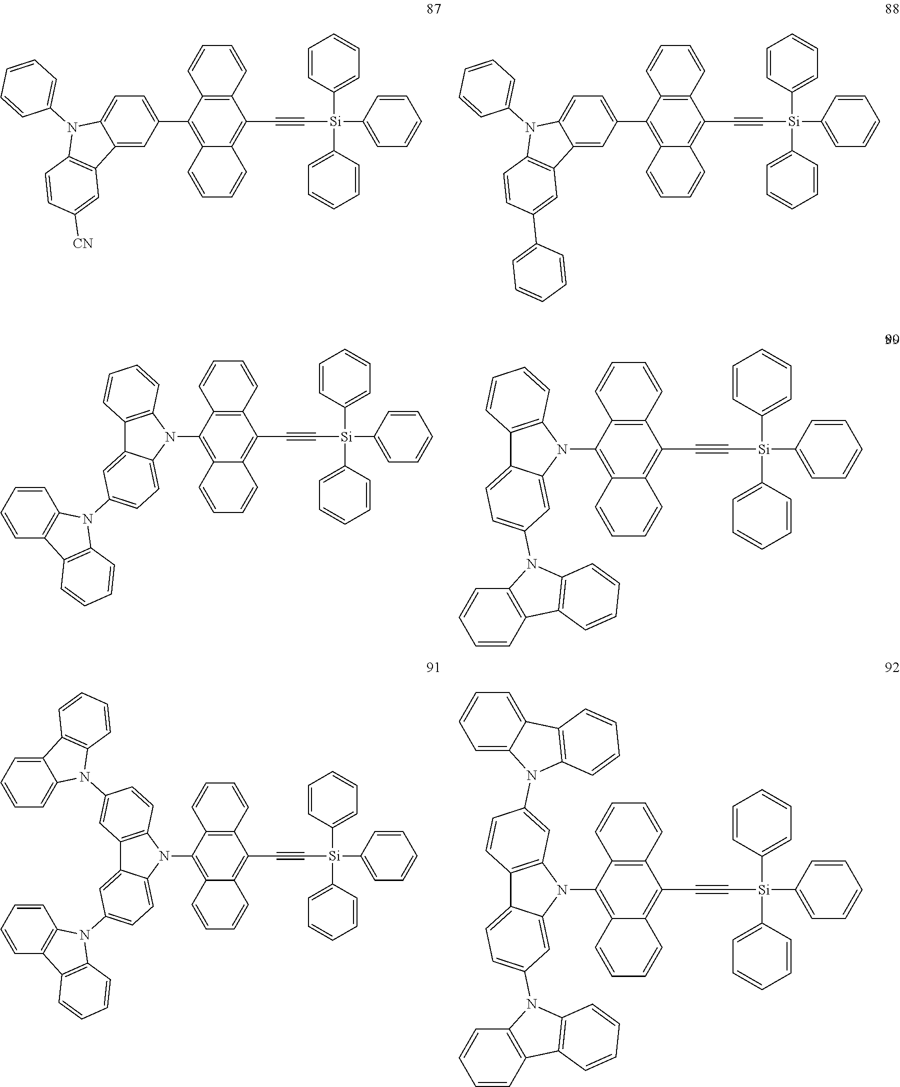

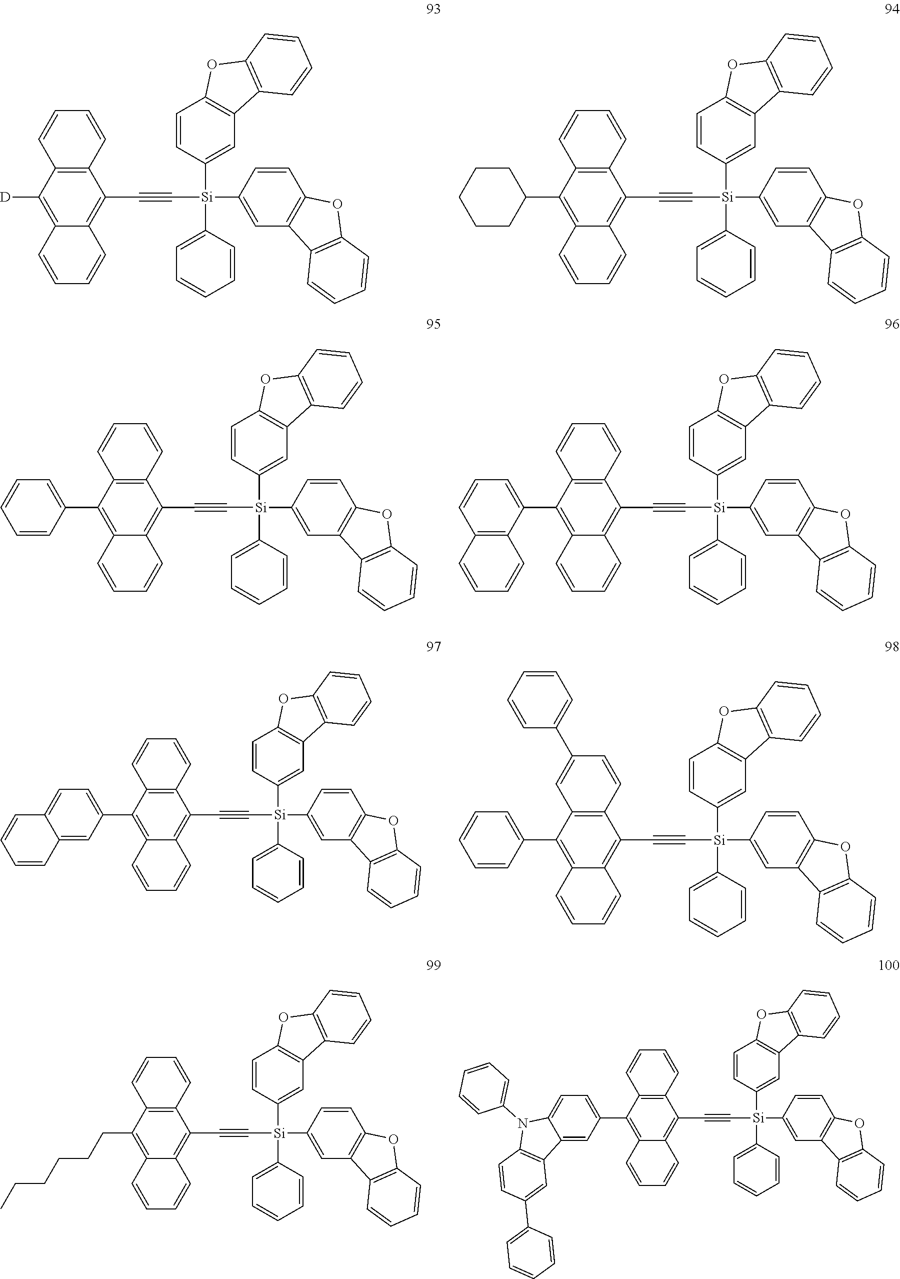

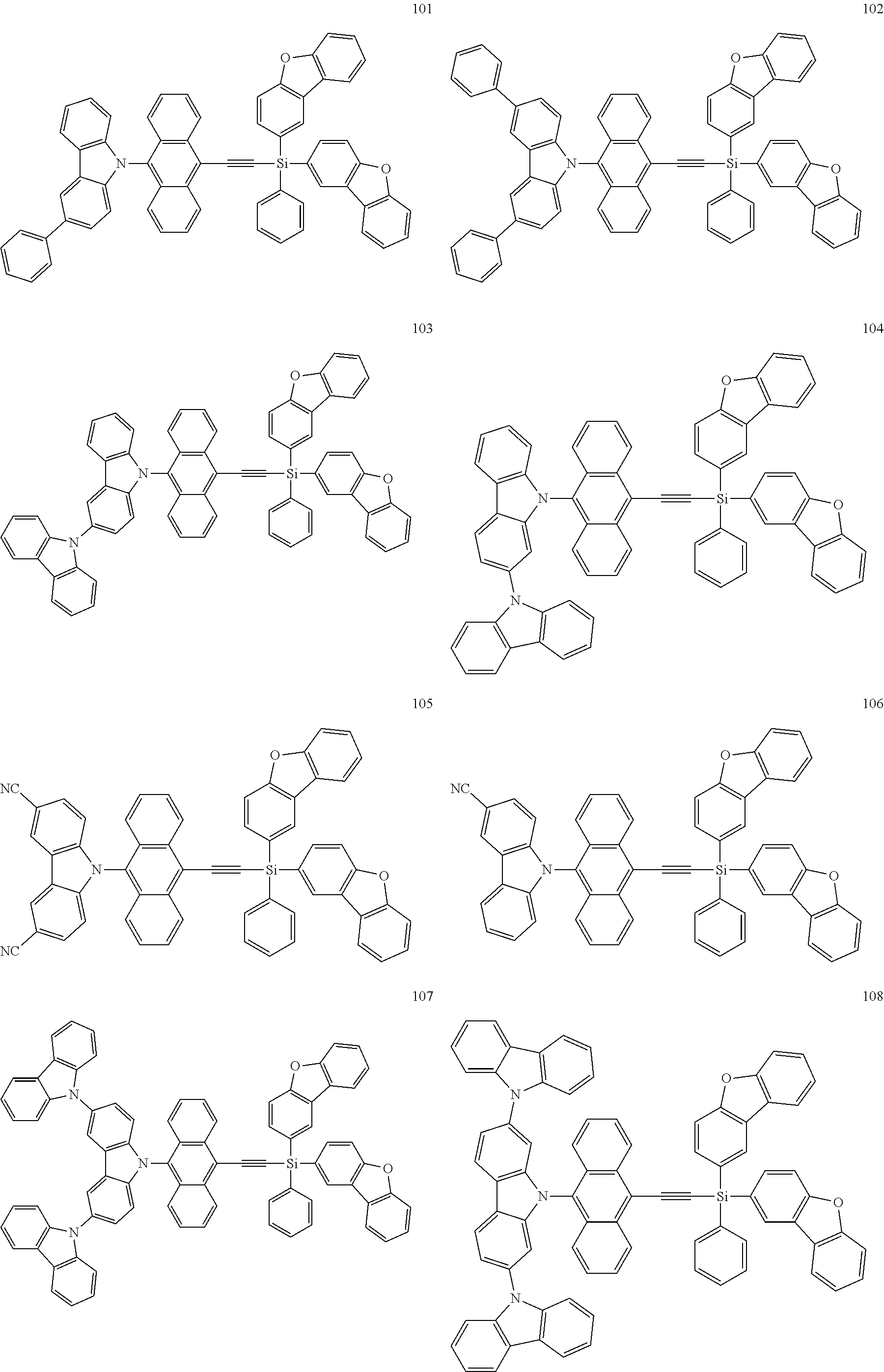

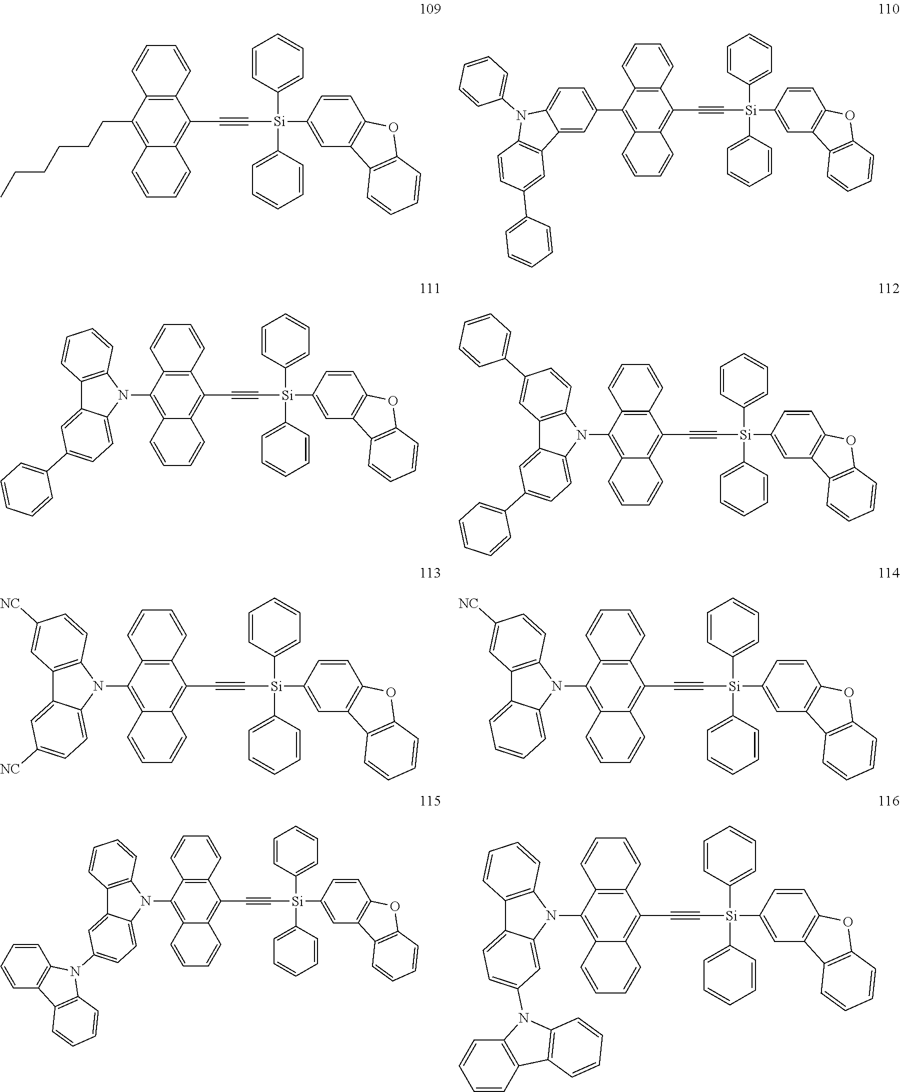

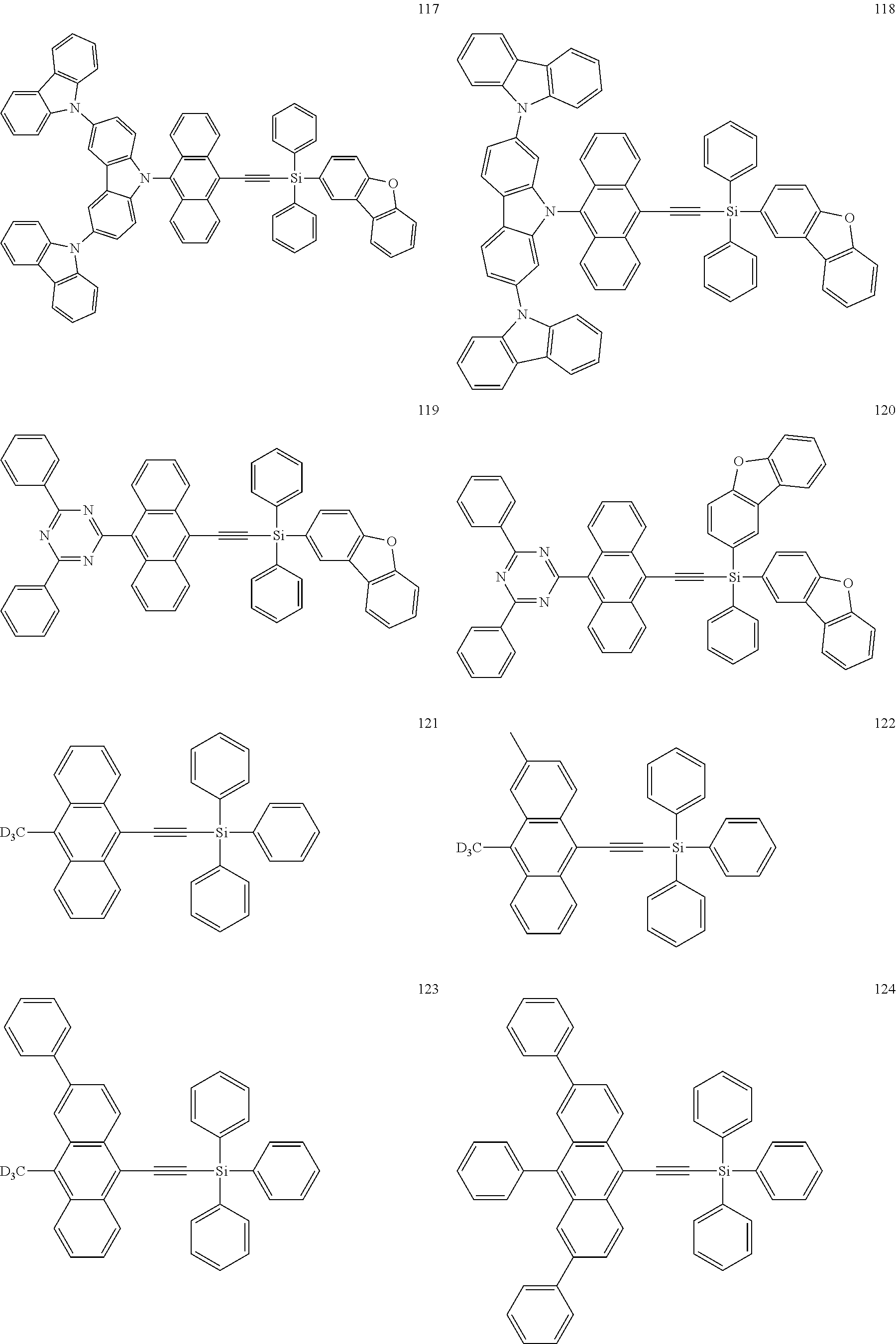

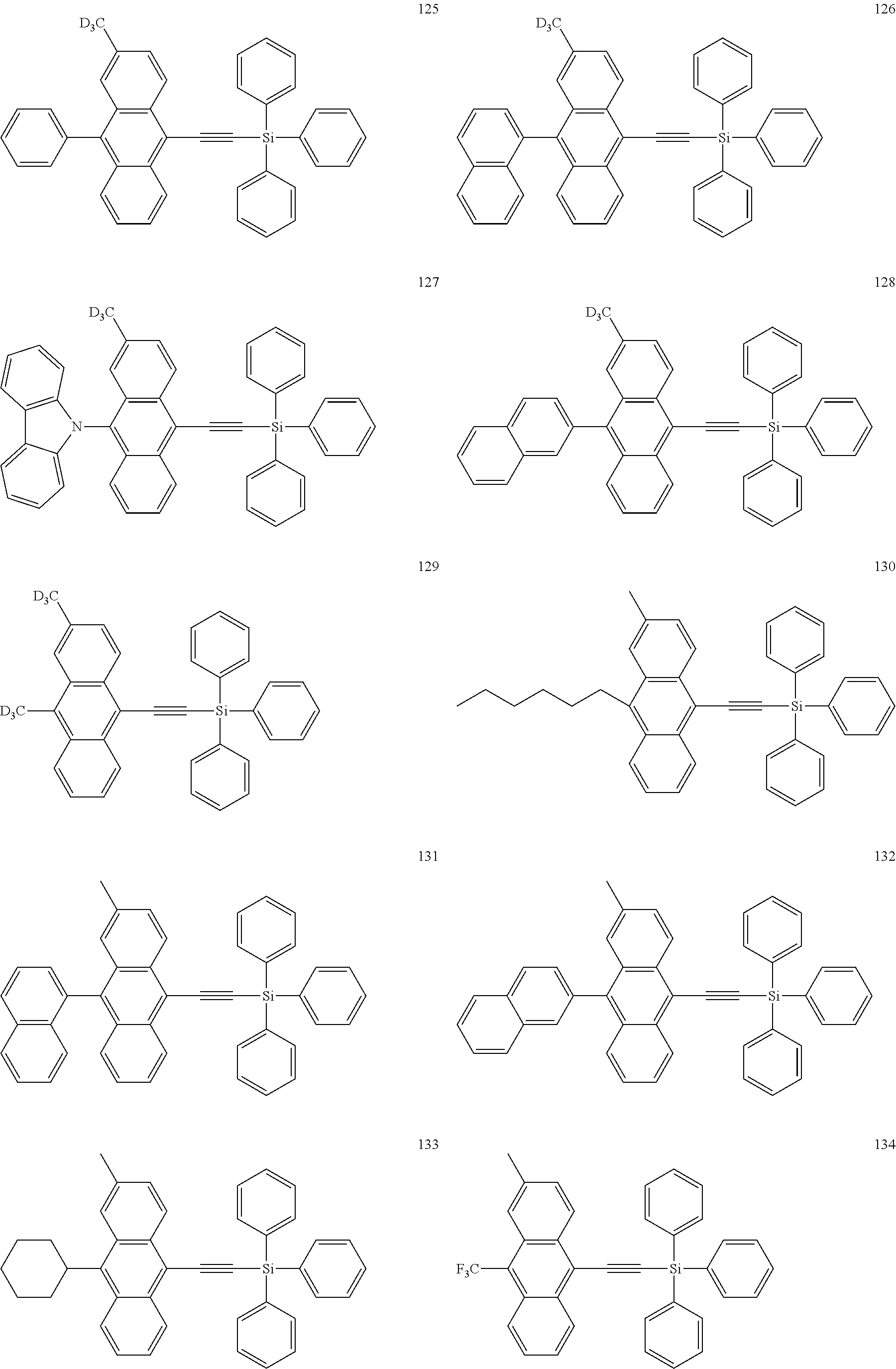

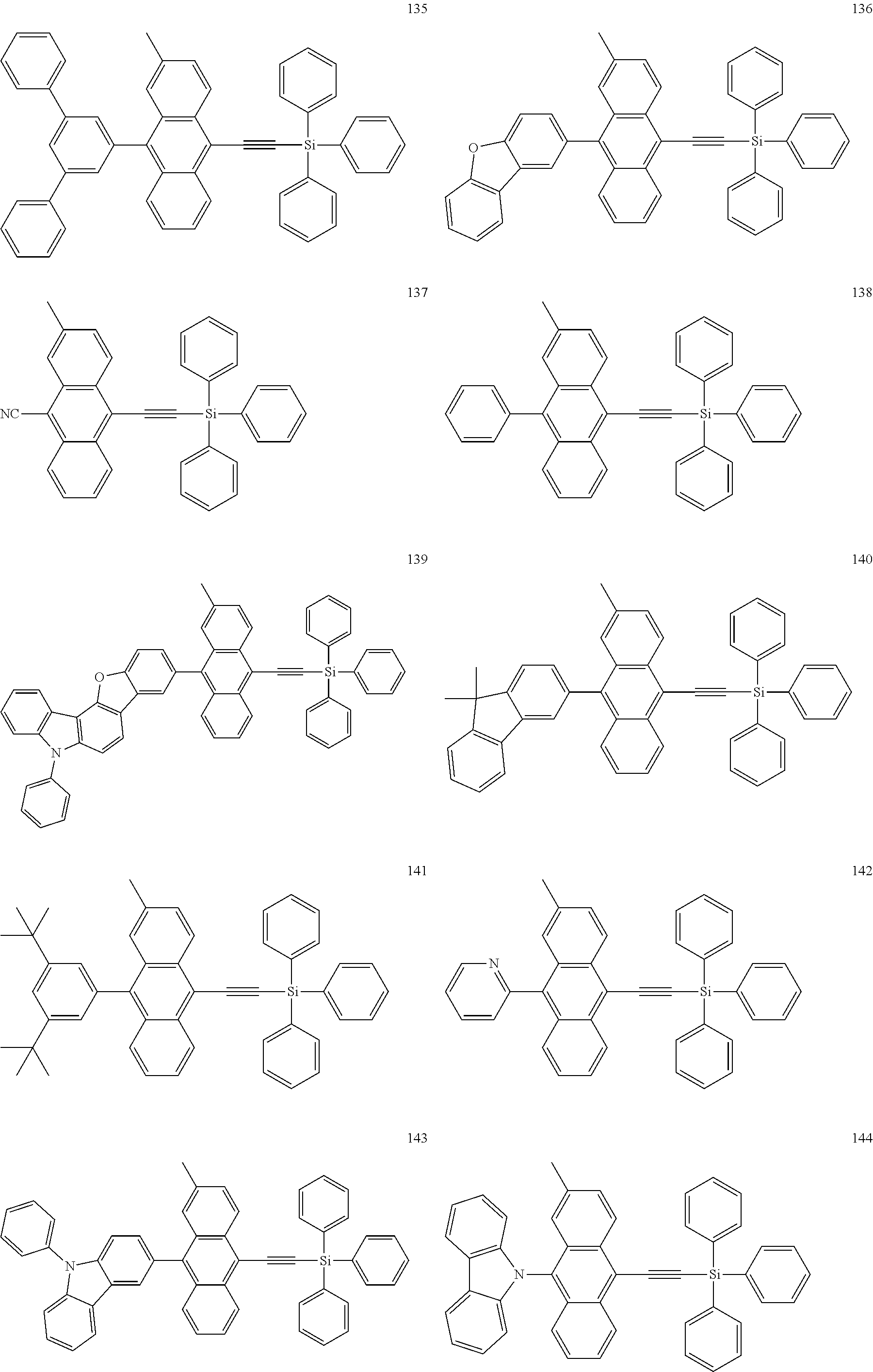

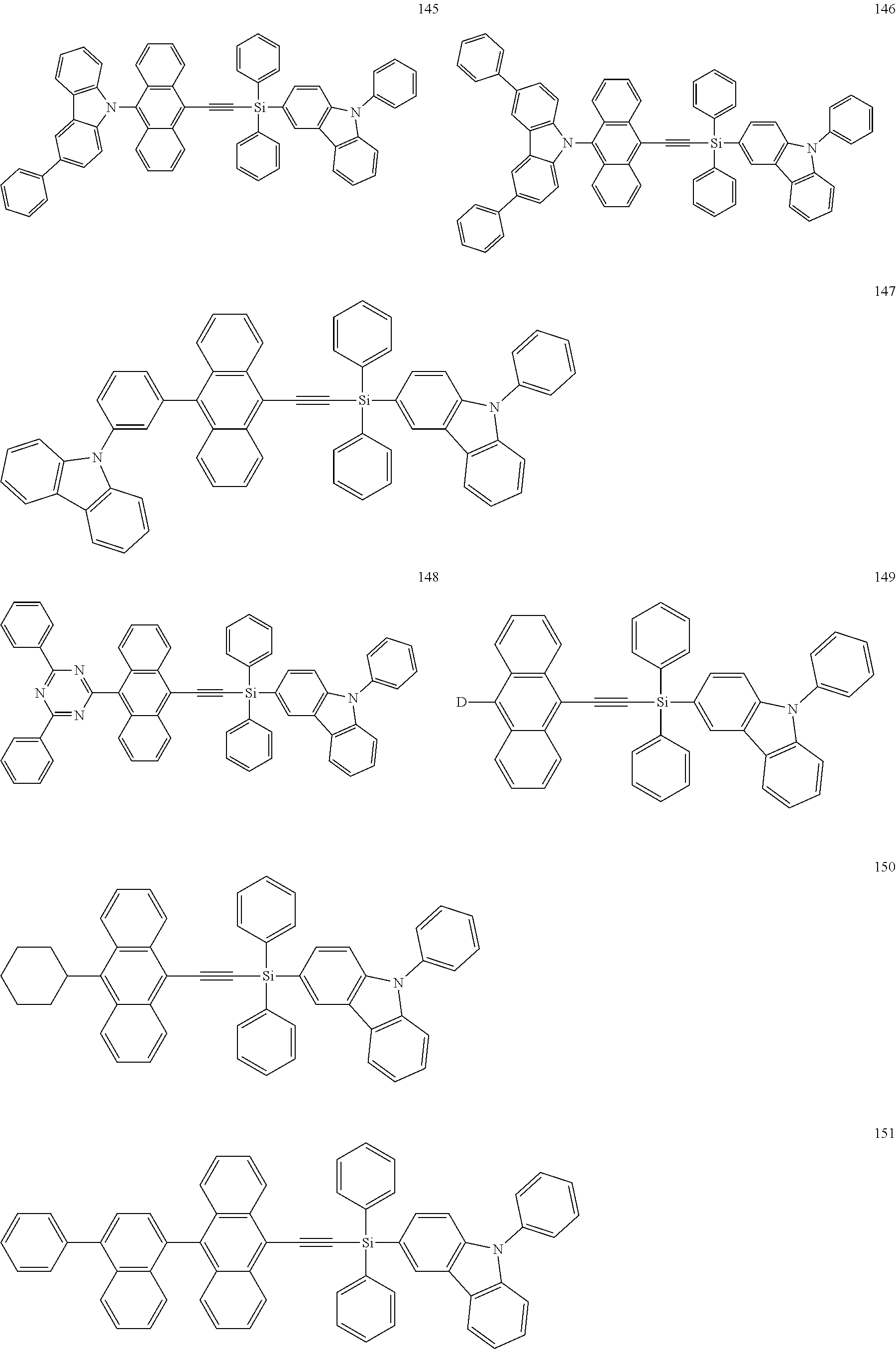

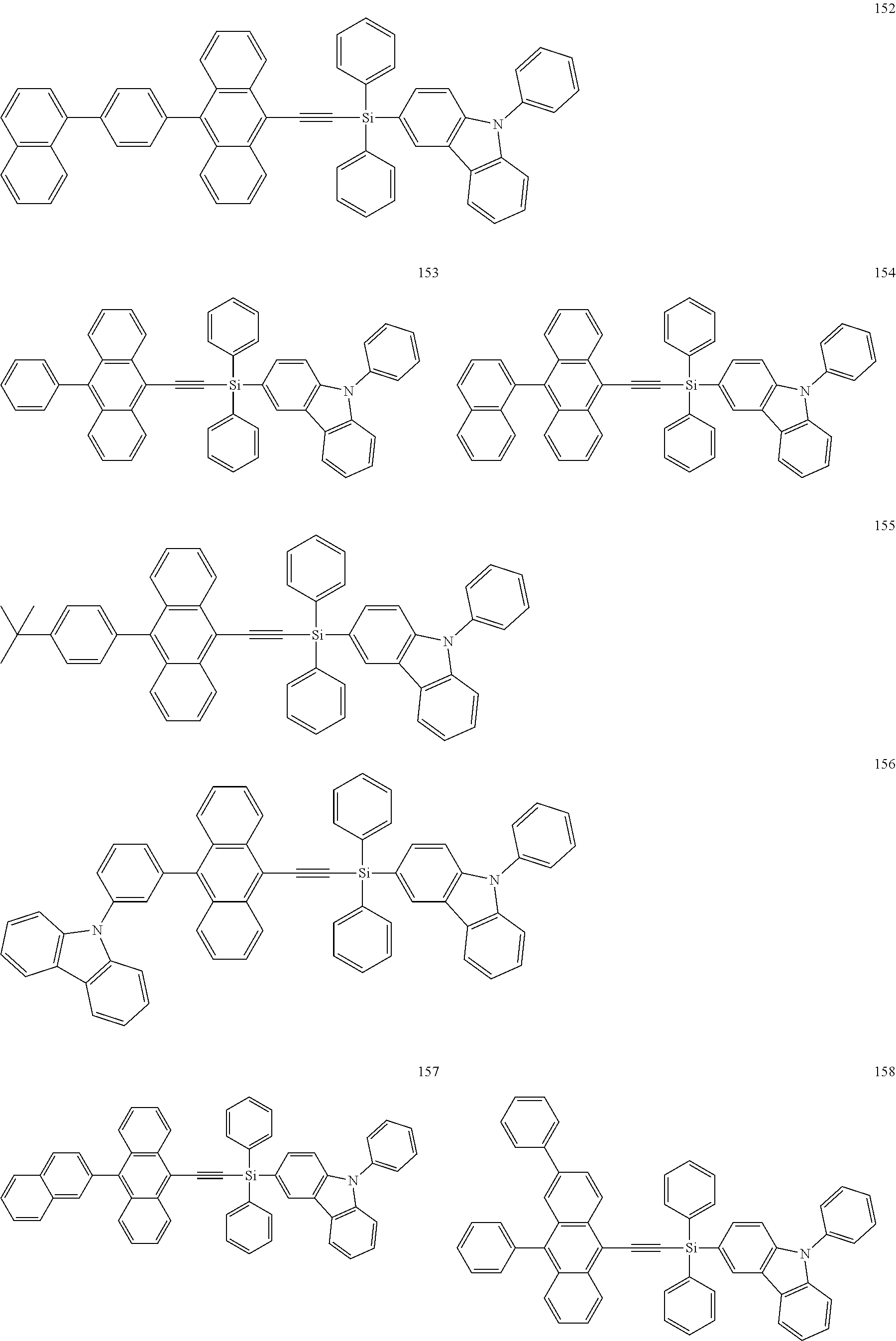

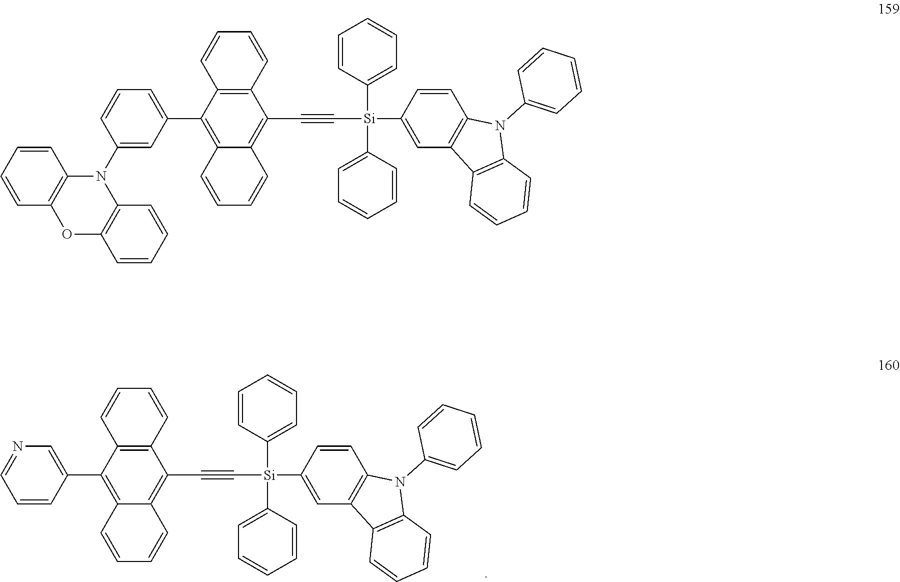 [SMILES: C(#C[Si](c1ccccc1)(c1ccccc1)c1ccc2c(c1)c1ccccc1n2-c1ccccc1)c1c2ccccc2c(-c2cccc(N3c4ccccc4Oc4ccccc43)c2)c2ccccc12 ; C(#C[Si](c1ccccc1)(c1ccccc1)c1ccc2c(c1)c1ccccc1n2-c1ccccc1)c1c2ccccc2c(-c2cccnc2)c2ccccc12]

In an embodiment, the fluorescent host may be represented by Formula FH-2:

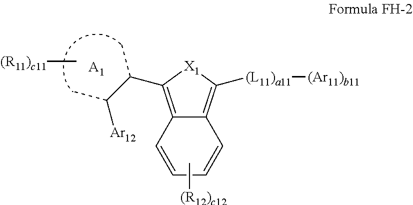

wherein, in Formula FH-2,
X₁ may be O or S,
A₁ may be a C₅-C₆₀ carbocyclic group or a C₁-C₆₀ heterocyclic group,
L₁₁ may be an unsubstituted or substituted C₅-C₆₀ carbocyclic group or an unsubstituted or substituted C₁-C₆₀ heterocyclic group,
a11 may be an integer from 0 to 3,
Ar₁₁ and Ar₁₂ may each independently be a C₆-C₆₀ aryl group, a C₁-C₆₀ heteroaryl group, a monovalent non-aromatic condensed polycyclic group, or a monovalent non-aromatic condensed heteropolycyclic group, each unsubstituted or substituted with at least one Rₐ,
b11 may be an integer from 1 to 5,
R₁₁, R₁₂, and Ra may each independently be hydrogen, deuterium, —F, —Cl, —Br, —I, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a substituted or unsubstituted C₁-C₆₀ alkyl group, a substituted or unsubstituted C₂-C₆₀ alkenyl group, a substituted or unsubstituted C₂-C₆₀ alkynyl group, a substituted or unsubstituted C₁-C₆₀ alkoxy group, a substituted or unsubstituted C₃-C₁₀ cycloalkyl group, a substituted or unsubstituted C₁-C₁₀ heterocycloalkyl group, a substituted or unsubstituted C₃-C₁₀ cycloalkenyl group, a substituted or unsubstituted C₂-C₁₀ heterocycloalkenyl group, a substituted or unsubstituted C₆-C₆₀ aryl group, a substituted or unsubstituted C₆-C₆₀ aryloxy group, a substituted or unsubstituted C₆-C₆₀ arylthio group, a substituted or unsubstituted C₁-C₆₀ heteroaryl group, a substituted or unsubstituted monovalent non-aromatic condensed polycyclic group, a substituted or unsubstituted monovalent non-aromatic condensed heteropolycyclic group, —Si(Q₁)(Q₂)(Q₃), —N(Q₄)(Q₅), or —B(Q₆)(Q₇),
c11 may be an integer from 1 to 20,
c12 may be an integer from 1 to 4,
when c11 is 2 or greater, two neighboring R₁₁(s) may optionally be combined with each other to form a substituted or unsubstituted C₅-C₃₀ carbocyclic group or a substituted or unsubstituted C₁-C₃₀ heterocyclic group,
when c12 is 2 or greater, two neighboring R₁₂(s) may optionally be combined with each other to form a substituted or unsubstituted C₅-C₃₀ carbocyclic group or a substituted or unsubstituted C₁-C₃₀ heterocyclic group,
A₁ and Ar₁₂ may optionally be linked to each other via a first linking group a single bond, *—Ar₃₁-′, *—[C(R₃₁)(R₃₂)]k11-*′, *—C(R₃₁)=*′, *═C(R₃₁)—*′, *—C(R₃₁)═C(R₃₂)—*′, *—C(═O)—′, *—C(═S)—′, *—N(R₃₁)—*′, *—P(R₃₁)—*′, *—[Si(R₃₁)(R₃₂)]k11-*′, or *—P(R₃₁)(R₃₂)—′ to form a substituted or unsubstituted C₅-C₃₀ carbocyclic group or a substituted or unsubstituted C₁-C₃₀ heterocyclic group,
A₃₁ may be a C₅-C₃₀ carbocyclic group,
R₃₁ and R₃₂ may each be the same as described in connection with R11, and
k11 may be 1, 2, 3, and 4.

In an embodiment, the fluorescent host represented by Formula FH-2 may be compounds of Group FH2, but embodiments of the present disclosure are not limited thereto:

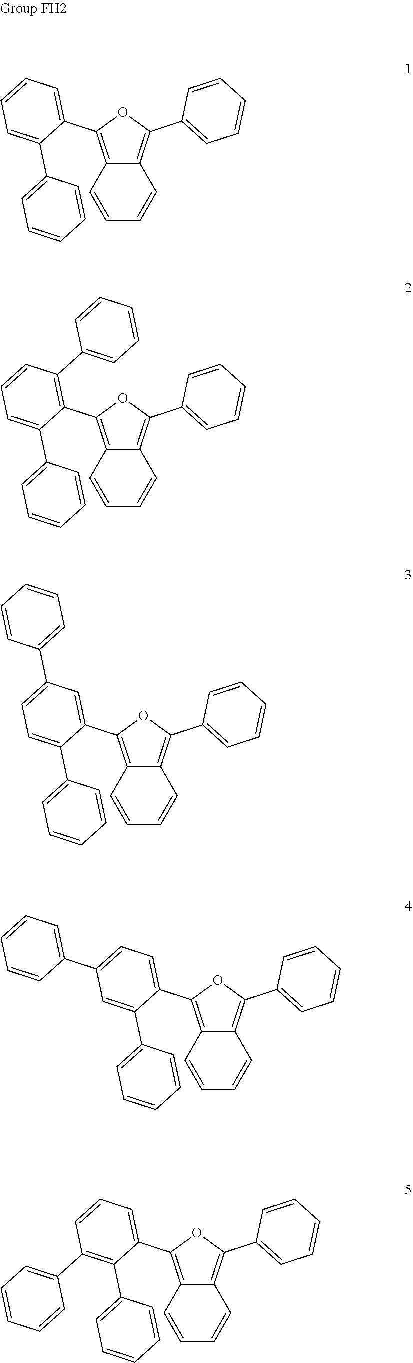 [SMILES: c1ccc(-c2ccccc2-c2oc(-c3ccccc3)c3ccccc23)cc1 ; c1ccc(-c2cccc(-c3ccccc3)c2-c2oc(-c3ccccc3)c3ccccc23)cc1 ; c1ccc(-c2ccc(-c3ccccc3)c(-c3oc(-c4ccccc4)c4ccccc34)c2)cc1 ; c1ccc(-c2ccc(-c3oc(-c4ccccc4)c4ccccc34)c(-c3ccccc3)c2)cc1 ; c1ccc(-c2cccc(-c3oc(-c4ccccc4)c4ccccc34)c2-c2ccccc2)cc1]

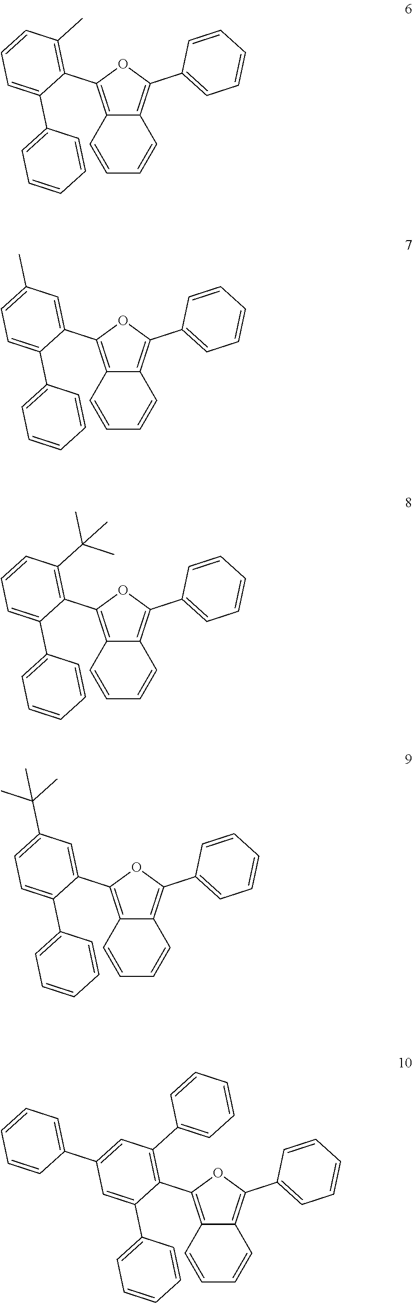 [SMILES: Cc1cccc(-c2ccccc2)c1-c1oc(-c2ccccc2)c2ccccc12 ; Cc1ccc(-c2ccccc2)c(-c2oc(-c3ccccc3)c3ccccc23)c1 ; CC(C)(C)c1cccc(-c2ccccc2)c1-c1oc(-c2ccccc2)c2ccccc12 ; CC(C)(C)c1ccc(-c2ccccc2)c(-c2oc(-c3ccccc3)c3ccccc23)c1 ; c1ccc(-c2cc(-c3ccccc3)c(-c3oc(-c4ccccc4)c4ccccc34)c(-c3ccccc3)c2)cc1]

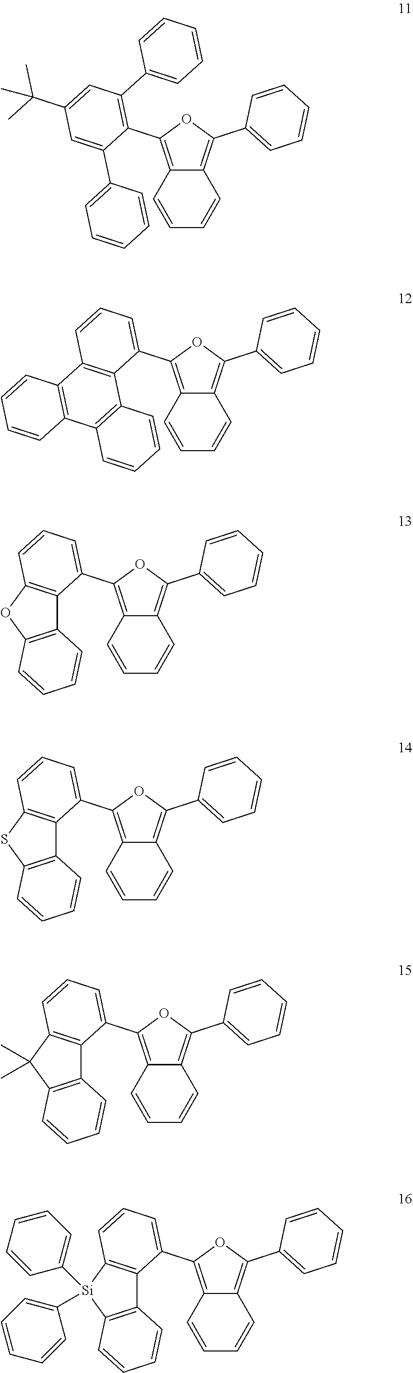 [SMILES: CC(C)(C)c1cc(-c2ccccc2)c(-c2oc(-c3ccccc3)c3ccccc23)c(-c2ccccc2)c1 ; c1ccc(-c2oc(-c3cccc4c5ccccc5c5ccccc5c34)c3ccccc23)cc1 ; c1ccc(-c2oc(-c3cccc4oc5ccccc5c34)c3ccccc23)cc1 ; c1ccc(-c2oc(-c3cccc4sc5ccccc5c34)c3ccccc23)cc1 ; CC1(C)c2ccccc2-c2c(-c3oc(-c4ccccc4)c4ccccc34)cccc21 ; c1ccc(-c2oc(-c3cccc4c3-c3ccccc3[Si]4(c3ccccc3)c3ccccc3)c3ccccc23)cc1]

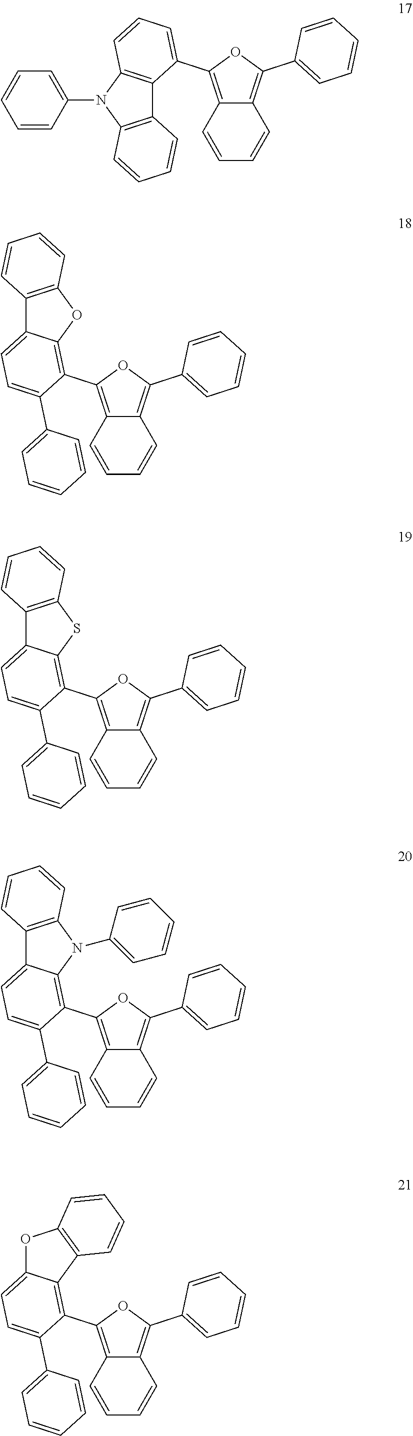 [SMILES: c1ccc(-c2oc(-c3cccc4c3c3ccccc3n4-c3ccccc3)c3ccccc23)cc1 ; c1ccc(-c2ccc3c(oc4ccccc43)c2-c2oc(-c3ccccc3)c3ccccc23)cc1 ; c1ccc(-c2ccc3c(sc4ccccc43)c2-c2oc(-c3ccccc3)c3ccccc23)cc1 ; c1ccc(-c2ccc3c4ccccc4n(-c4ccccc4)c3c2-c2oc(-c3ccccc3)c3ccccc23)cc1 ; c1ccc(-c2ccc3oc4ccccc4c3c2-c2oc(-c3ccccc3)c3ccccc23)cc1]

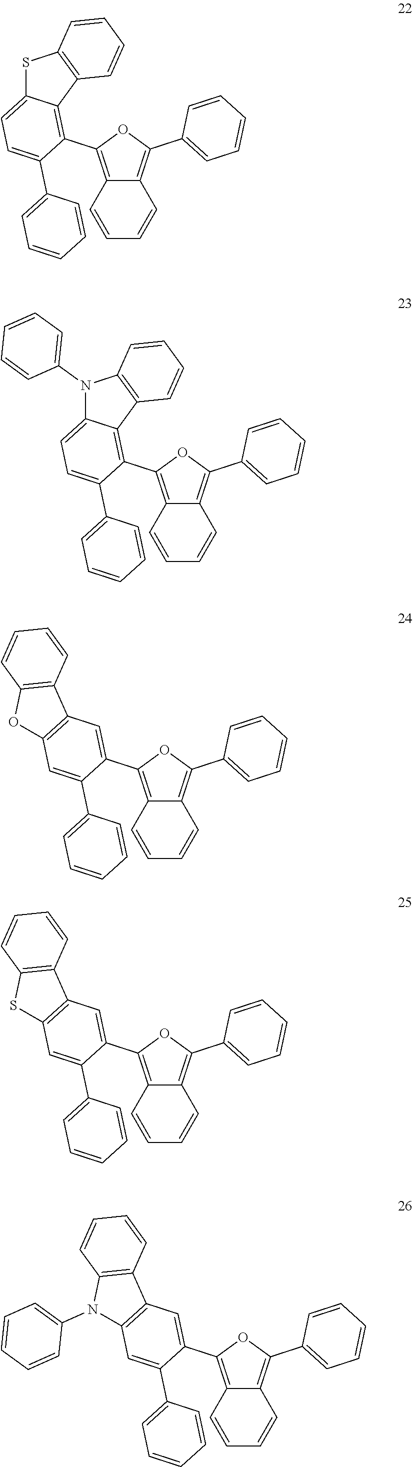 [SMILES: c1ccc(-c2ccc3sc4ccccc4c3c2-c2oc(-c3ccccc3)c3ccccc23)cc1 ; c1ccc(-c2ccc3c(c2-c2oc(-c4ccccc4)c4ccccc24)c2ccccc2n3-c2ccccc2)cc1 ; c1ccc(-c2cc3oc4ccccc4c3cc2-c2oc(-c3ccccc3)c3ccccc23)cc1 ; c1ccc(-c2cc3sc4ccccc4c3cc2-c2oc(-c3ccccc3)c3ccccc23)cc1 ; c1ccc(-c2cc3c(cc2-c2oc(-c4ccccc4)c4ccccc24)c2ccccc2n3-c2ccccc2)cc1]

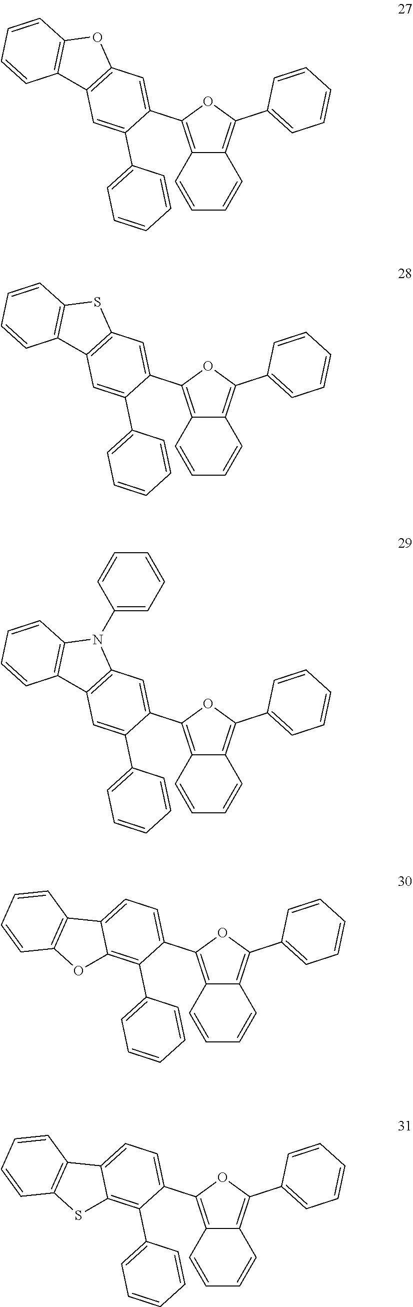 [SMILES: c1ccc(-c2cc3c(cc2-c2oc(-c4ccccc4)c4ccccc24)oc2ccccc23)cc1 ; c1ccc(-c2cc3c(cc2-c2oc(-c4ccccc4)c4ccccc24)sc2ccccc23)cc1 ; c1ccc(-c2cc3c4ccccc4n(-c4ccccc4)c3cc2-c2oc(-c3ccccc3)c3ccccc23)cc1 ; c1ccc(-c2oc(-c3ccc4c(oc5ccccc54)c3-c3ccccc3)c3ccccc23)cc1 ; c1ccc(-c2oc(-c3ccc4c(sc5ccccc54)c3-c3ccccc3)c3ccccc23)cc1]

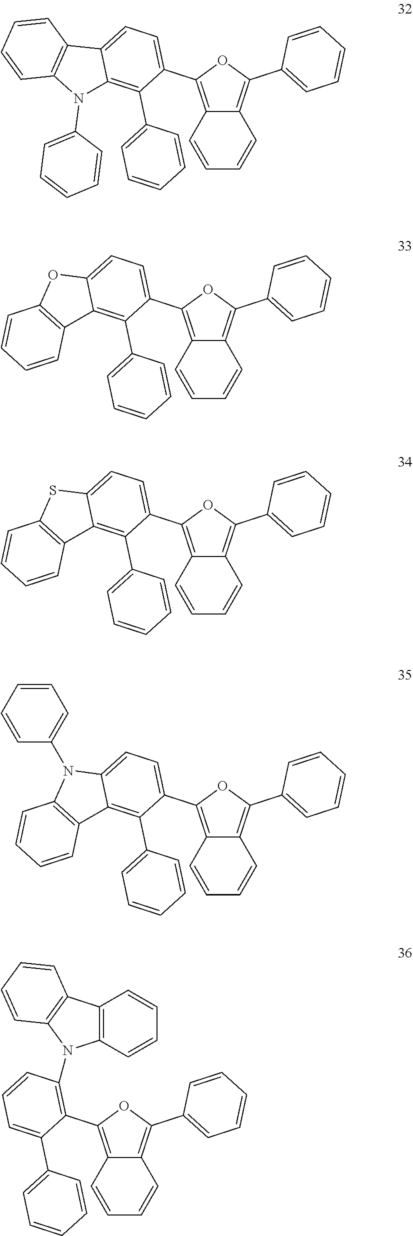 [SMILES: c1ccc(-c2oc(-c3ccc4c5ccccc5n(-c5ccccc5)c4c3-c3ccccc3)c3ccccc23)cc1 ; c1ccc(-c2oc(-c3ccc4oc5ccccc5c4c3-c3ccccc3)c3ccccc23)cc1 ; c1ccc(-c2oc(-c3ccc4sc5ccccc5c4c3-c3ccccc3)c3ccccc23)cc1 ; c1ccc(-c2oc(-c3ccc4c(c3-c3ccccc3)c3ccccc3n4-c3ccccc3)c3ccccc23)cc1 ; c1ccc(-c2cccc(-n3c4ccccc4c4ccccc43)c2-c2oc(-c3ccccc3)c3ccccc23)cc1]

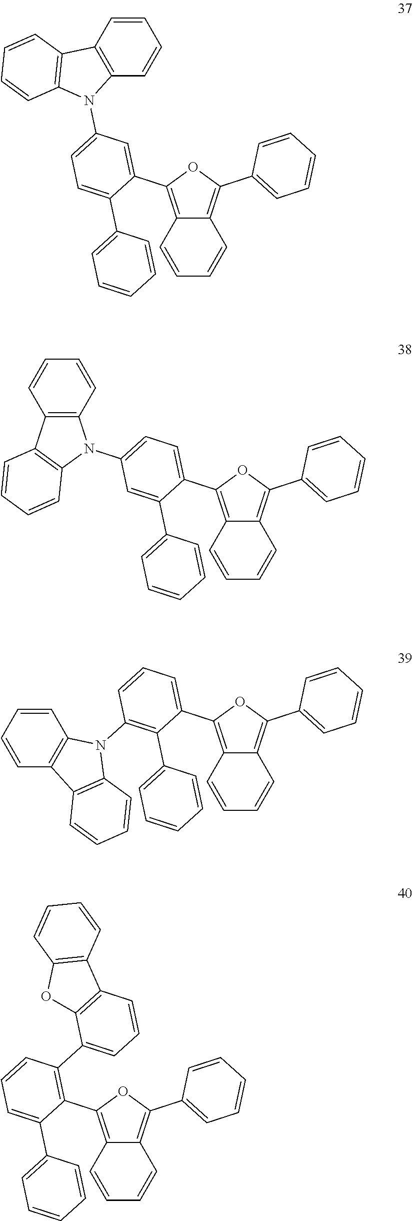 [SMILES: c1ccc(-c2ccc(-n3c4ccccc4c4ccccc43)cc2-c2oc(-c3ccccc3)c3ccccc23)cc1 ; c1ccc(-c2cc(-n3c4ccccc4c4ccccc43)ccc2-c2oc(-c3ccccc3)c3ccccc23)cc1 ; c1ccc(-c2c(-c3oc(-c4ccccc4)c4ccccc34)cccc2-n2c3ccccc3c3ccccc32)cc1 ; c1ccc(-c2cccc(-c3cccc4c3oc3ccccc34)c2-c2oc(-c3ccccc3)c3ccccc23)cc1]

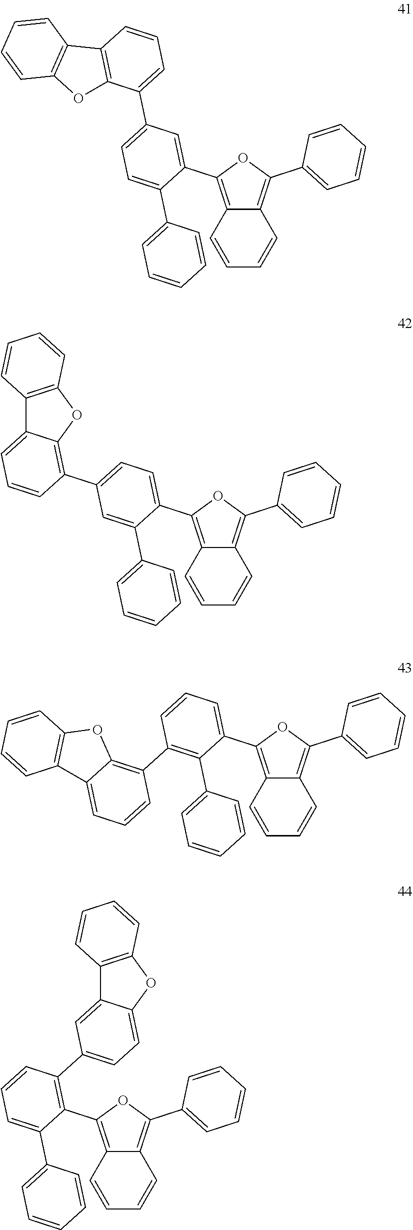 [SMILES: c1ccc(-c2ccc(-c3cccc4c3oc3ccccc34)cc2-c2oc(-c3ccccc3)c3ccccc23)cc1 ; c1ccc(-c2cc(-c3cccc4c3oc3ccccc34)ccc2-c2oc(-c3ccccc3)c3ccccc23)cc1 ; c1ccc(-c2c(-c3oc(-c4ccccc4)c4ccccc34)cccc2-c2cccc3c2oc2ccccc23)cc1 ; c1ccc(-c2cccc(-c3ccc4oc5ccccc5c4c3)c2-c2oc(-c3ccccc3)c3ccccc23)cc1]

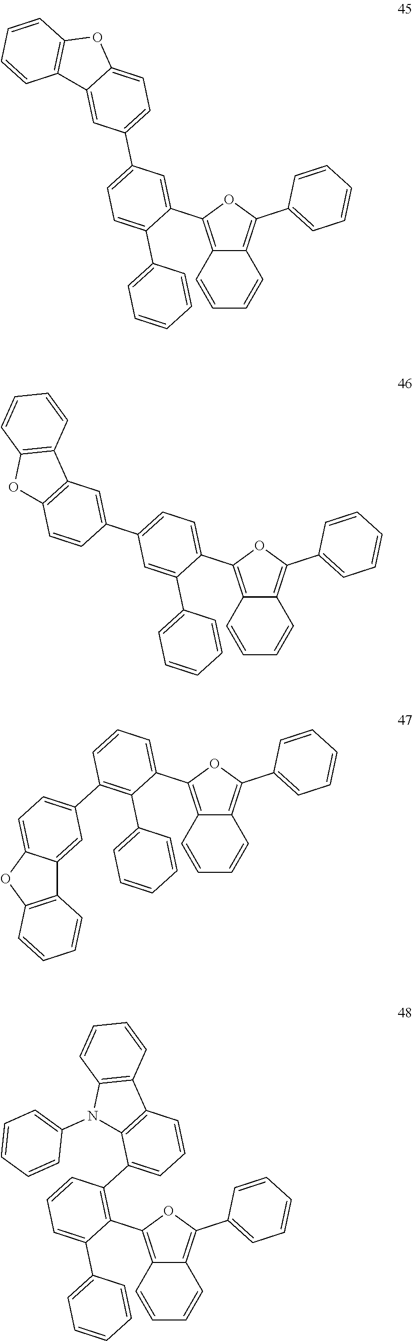 [SMILES: c1ccc(-c2ccc(-c3ccc4oc5ccccc5c4c3)cc2-c2oc(-c3ccccc3)c3ccccc23)cc1 ; c1ccc(-c2cc(-c3ccc4oc5ccccc5c4c3)ccc2-c2oc(-c3ccccc3)c3ccccc23)cc1 ; c1ccc(-c2c(-c3ccc4oc5ccccc5c4c3)cccc2-c2oc(-c3ccccc3)c3ccccc23)cc1 ; c1ccc(-c2cccc(-c3cccc4c5ccccc5n(-c5ccccc5)c34)c2-c2oc(-c3ccccc3)c3ccccc23)cc1]

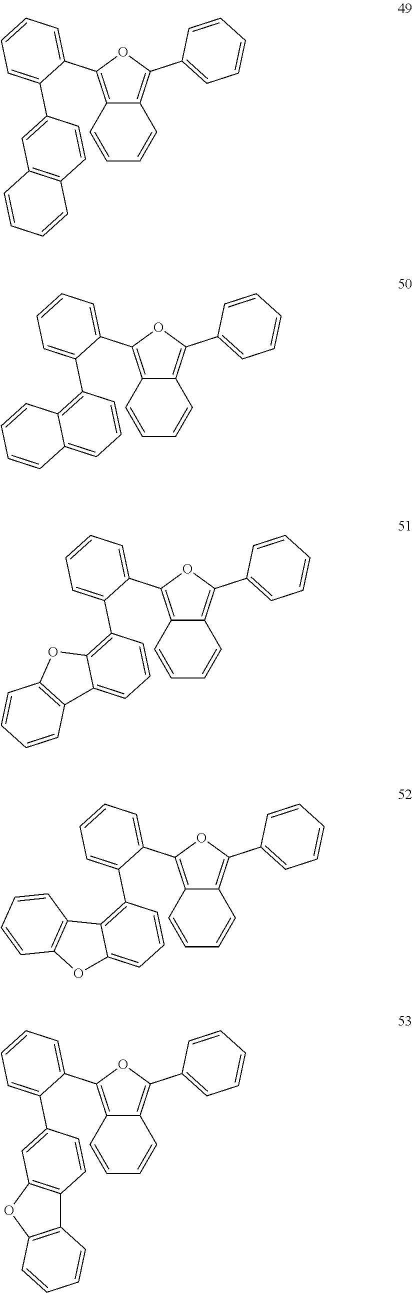 [SMILES: c1ccc(-c2oc(-c3ccccc3-c3ccc4ccccc4c3)c3ccccc23)cc1 ; c1ccc(-c2oc(-c3ccccc3-c3cccc4ccccc34)c3ccccc23)cc1 ; c1ccc(-c2oc(-c3ccccc3-c3cccc4c3oc3ccccc34)c3ccccc23)cc1 ; c1ccc(-c2oc(-c3ccccc3-c3cccc4oc5ccccc5c34)c3ccccc23)cc1 ; c1ccc(-c2oc(-c3ccccc3-c3ccc4c(c3)oc3ccccc34)c3ccccc23)cc1]

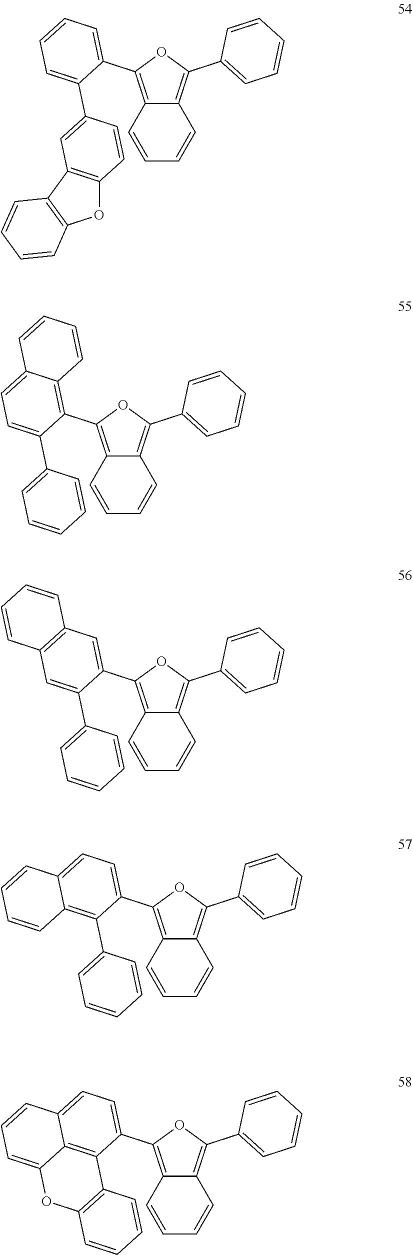 [SMILES: c1ccc(-c2oc(-c3ccccc3-c3ccc4oc5ccccc5c4c3)c3ccccc23)cc1 ; c1ccc(-c2ccc3ccccc3c2-c2oc(-c3ccccc3)c3ccccc23)cc1 ; c1ccc(-c2cc3ccccc3cc2-c2oc(-c3ccccc3)c3ccccc23)cc1 ; c1ccc(-c2c(-c3oc(-c4ccccc4)c4ccccc34)ccc3ccccc23)cc1 ; c1ccc(-c2oc(-c3ccc4cccc5c4c3-c3ccccc3O5)c3ccccc23)cc1]

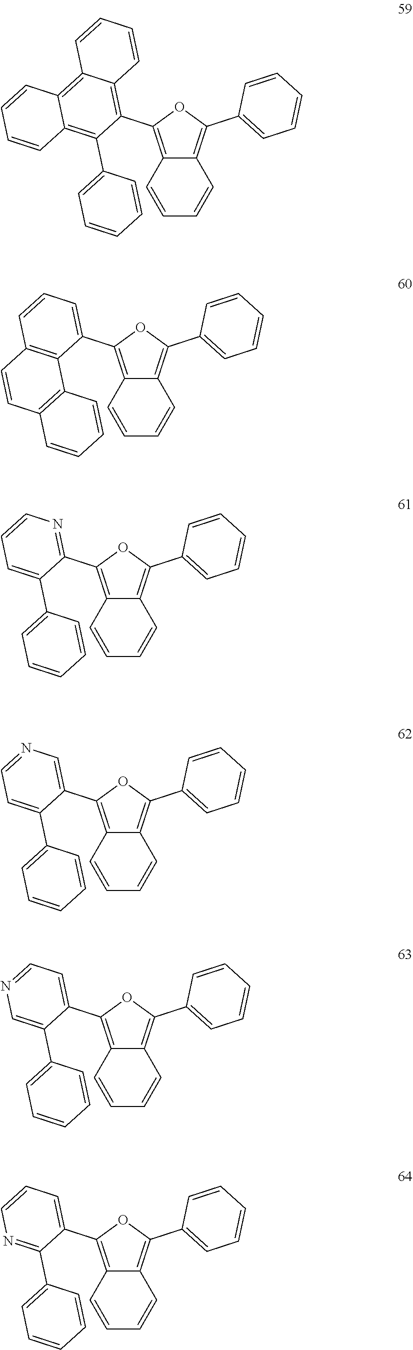 [SMILES: c1ccc(-c2oc(-c3c(-c4ccccc4)c4ccccc4c4ccccc34)c3ccccc23)cc1 ; c1ccc(-c2oc(-c3cccc4ccc5ccccc5c34)c3ccccc23)cc1 ; c1ccc(-c2cccnc2-c2oc(-c3ccccc3)c3ccccc23)cc1 ; c1ccc(-c2ccncc2-c2oc(-c3ccccc3)c3ccccc23)cc1 ; c1ccc(-c2cnccc2-c2oc(-c3ccccc3)c3ccccc23)cc1 ; c1ccc(-c2ncccc2-c2oc(-c3ccccc3)c3ccccc23)cc1]

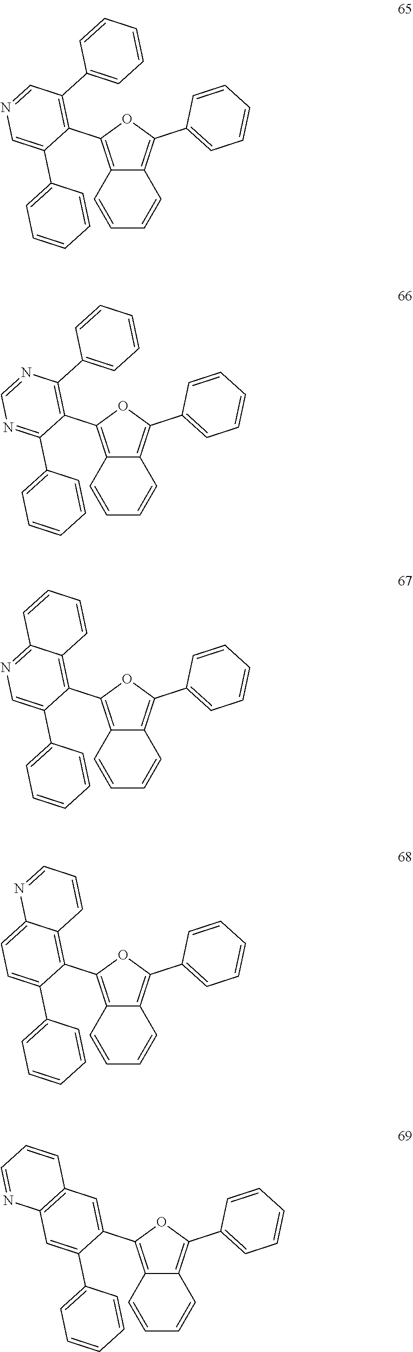 [SMILES: c1ccc(-c2cncc(-c3ccccc3)c2-c2oc(-c3ccccc3)c3ccccc23)cc1 ; c1ccc(-c2ncnc(-c3ccccc3)c2-c2oc(-c3ccccc3)c3ccccc23)cc1 ; c1ccc(-c2cnc3ccccc3c2-c2oc(-c3ccccc3)c3ccccc23)cc1 ; c1ccc(-c2ccc3ncccc3c2-c2oc(-c3ccccc3)c3ccccc23)cc1 ; c1ccc(-c2cc3ncccc3cc2-c2oc(-c3ccccc3)c3ccccc23)cc1]

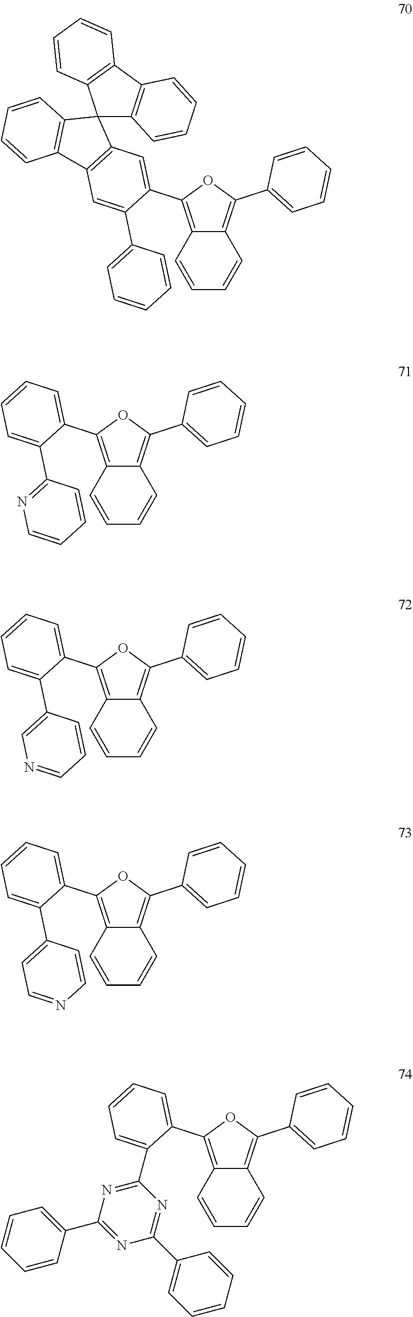 [SMILES: c1ccc(-c2cc3c(cc2-c2oc(-c4ccccc4)c4ccccc24)C2(c4ccccc4-c4ccccc42)c2ccccc2-3)cc1 ; c1ccc(-c2oc(-c3ccccc3-c3ccccn3)c3ccccc23)cc1 ; c1ccc(-c2oc(-c3ccccc3-c3cccnc3)c3ccccc23)cc1 ; c1ccc(-c2oc(-c3ccccc3-c3ccncc3)c3ccccc23)cc1 ; c1ccc(-c2nc(-c3ccccc3)nc(-c3ccccc3-c3oc(-c4ccccc4)c4ccccc34)n2)cc1]

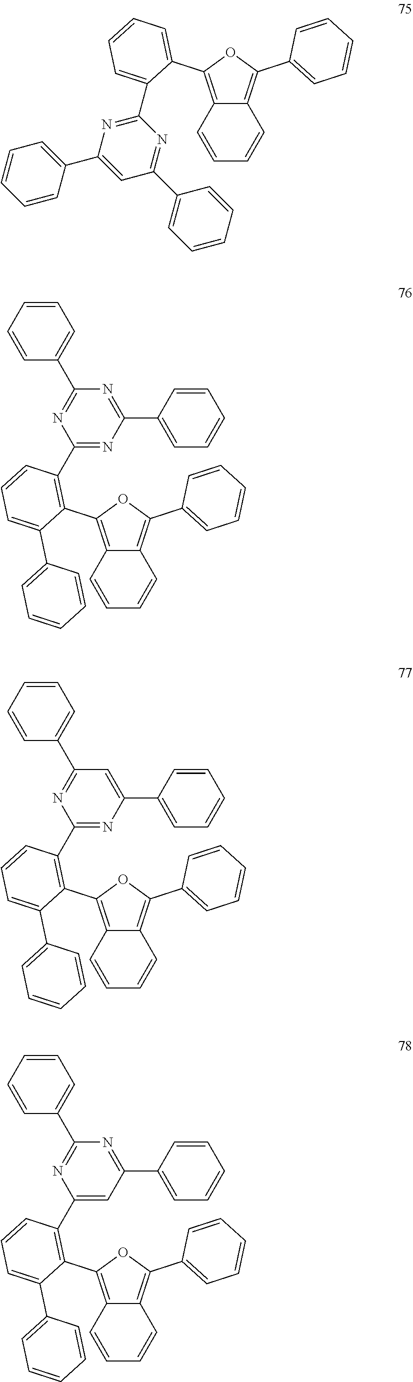 [SMILES: c1ccc(-c2cc(-c3ccccc3)nc(-c3ccccc3-c3oc(-c4ccccc4)c4ccccc34)n2)cc1 ; c1ccc(-c2nc(-c3ccccc3)nc(-c3cccc(-c4ccccc4)c3-c3oc(-c4ccccc4)c4ccccc34)n2)cc1 ; c1ccc(-c2cc(-c3ccccc3)nc(-c3cccc(-c4ccccc4)c3-c3oc(-c4ccccc4)c4ccccc34)n2)cc1 ; c1ccc(-c2cc(-c3cccc(-c4ccccc4)c3-c3oc(-c4ccccc4)c4ccccc34)nc(-c3ccccc3)n2)cc1]

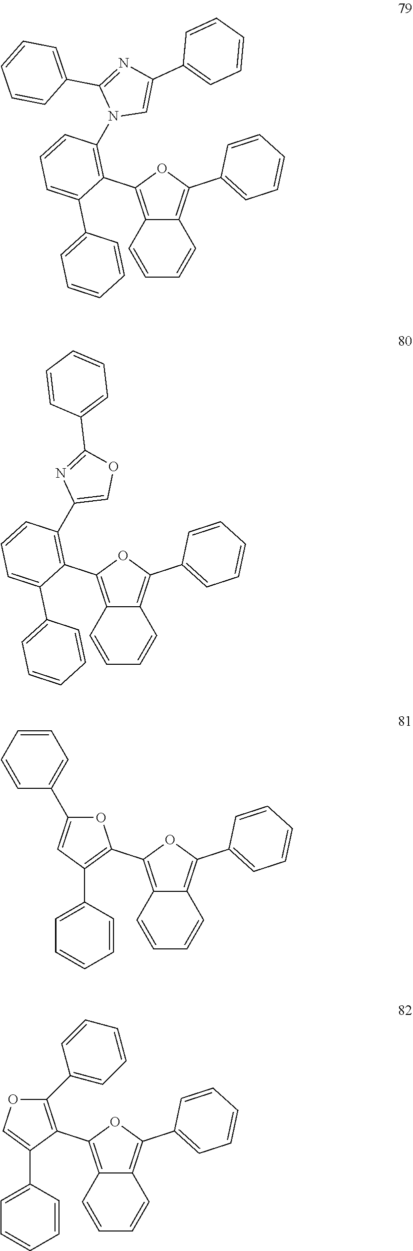 [SMILES: c1ccc(-c2cn(-c3cccc(-c4ccccc4)c3-c3oc(-c4ccccc4)c4ccccc34)c(-c3ccccc3)n2)cc1 ; c1ccc(-c2nc(-c3cccc(-c4ccccc4)c3-c3oc(-c4ccccc4)c4ccccc34)co2)cc1 ; c1ccc(-c2cc(-c3ccccc3)c(-c3oc(-c4ccccc4)c4ccccc34)o2)cc1 ; c1ccc(-c2coc(-c3ccccc3)c2-c2oc(-c3ccccc3)c3ccccc23)cc1]

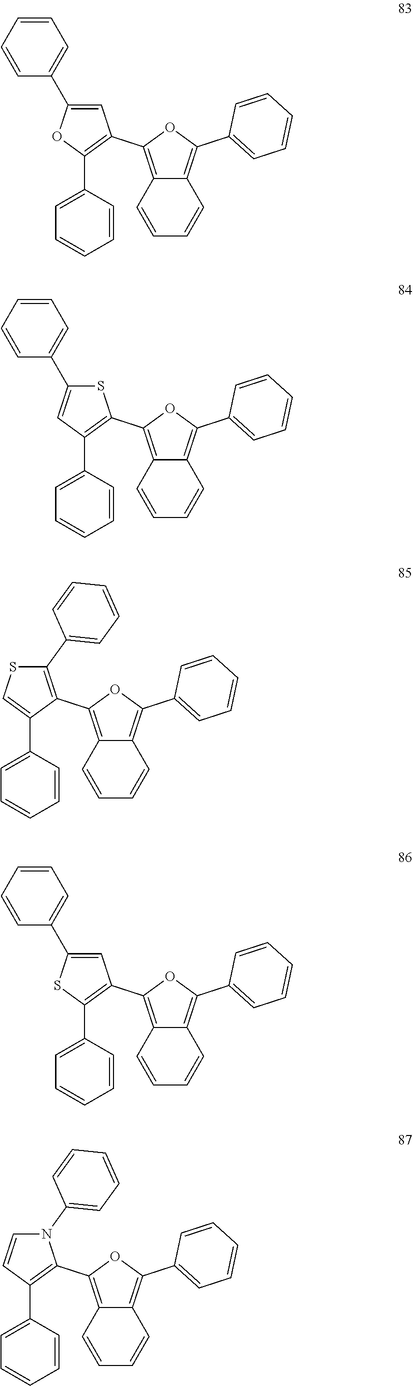

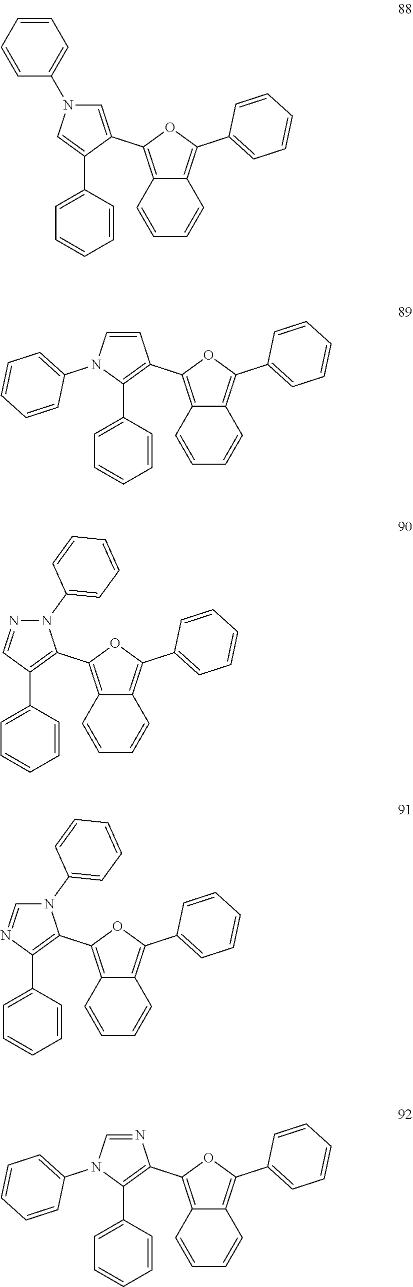 [SMILES: c1ccc(-c2cn(-c3ccccc3)cc2-c2oc(-c3ccccc3)c3ccccc23)cc1 ; c1ccc(-c2oc(-c3ccn(-c4ccccc4)c3-c3ccccc3)c3ccccc23)cc1 ; c1ccc(-c2cnn(-c3ccccc3)c2-c2oc(-c3ccccc3)c3ccccc23)cc1 ; c1ccc(-c2ncn(-c3ccccc3)c2-c2oc(-c3ccccc3)c3ccccc23)cc1 ; c1ccc(-c2oc(-c3ncn(-c4ccccc4)c3-c3ccccc3)c3ccccc23)cc1]

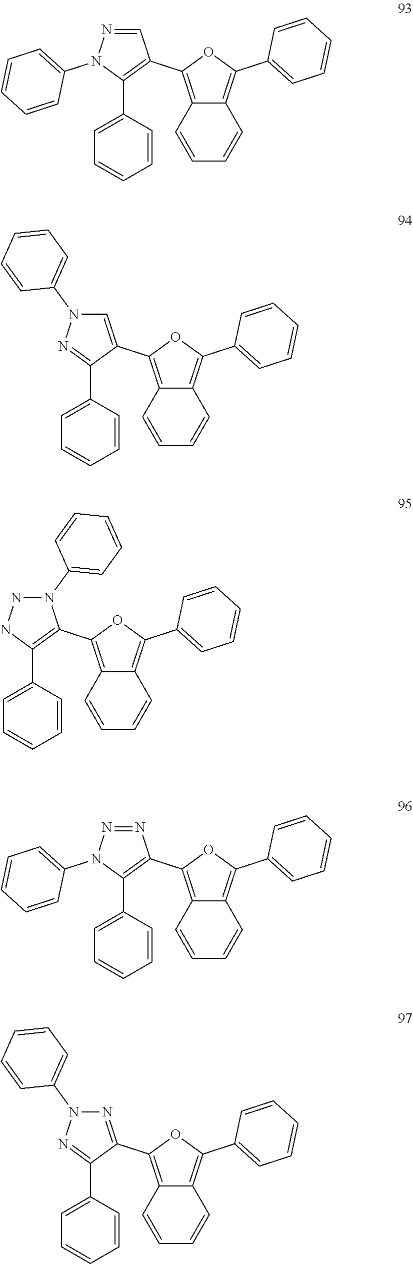 [SMILES: c1ccc(-c2oc(-c3cnn(-c4ccccc4)c3-c3ccccc3)c3ccccc23)cc1 ; c1ccc(-c2nn(-c3ccccc3)cc2-c2oc(-c3ccccc3)c3ccccc23)cc1 ; c1ccc(C2=C(c3oc(-c4ccccc4)c4ccccc34)N(c3ccccc3)NN2)cc1 ; c1ccc(-c2oc(-c3nnn(-c4ccccc4)c3-c3ccccc3)c3ccccc23)cc1 ; c1ccc(-c2nn(-c3ccccc3)nc2-c2oc(-c3ccccc3)c3ccccc23)cc1]

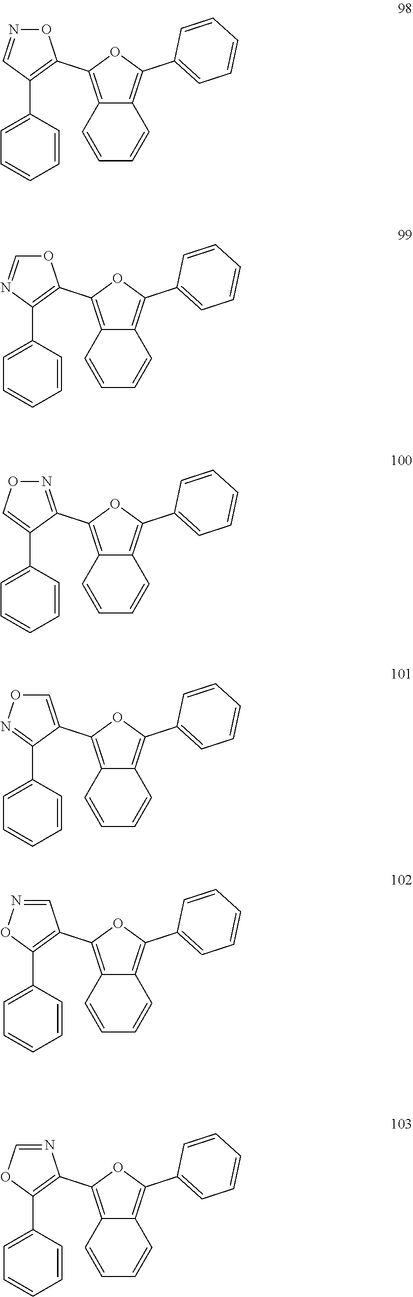 [SMILES: c1ccc(-c2cnoc2-c2oc(-c3ccccc3)c3ccccc23)cc1 ; c1ccc(-c2ncoc2-c2oc(-c3ccccc3)c3ccccc23)cc1 ; c1ccc(-c2conc2-c2oc(-c3ccccc3)c3ccccc23)cc1 ; c1ccc(-c2nocc2-c2oc(-c3ccccc3)c3ccccc23)cc1 ; c1ccc(-c2oncc2-c2oc(-c3ccccc3)c3ccccc23)cc1 ; c1ccc(-c2ocnc2-c2oc(-c3ccccc3)c3ccccc23)cc1]

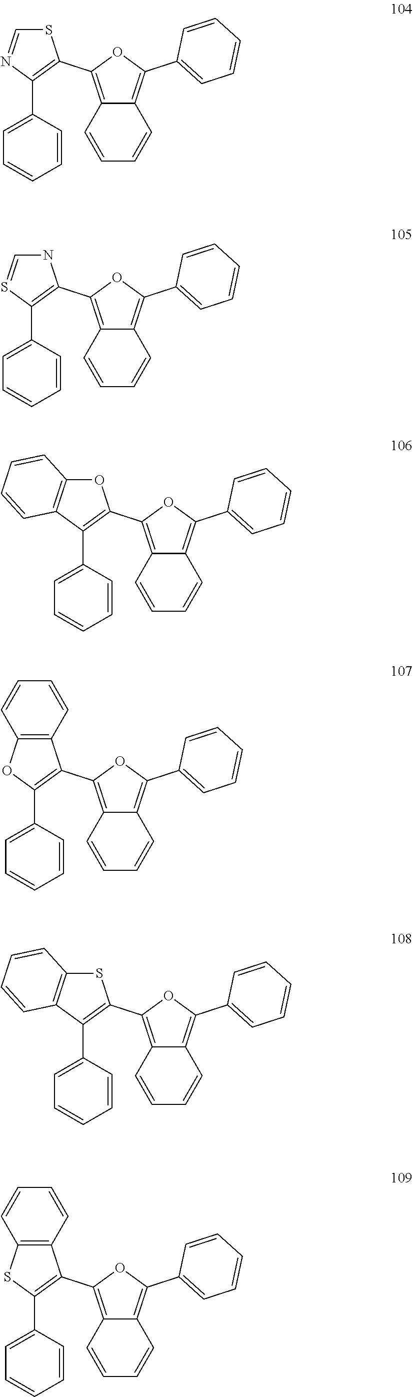

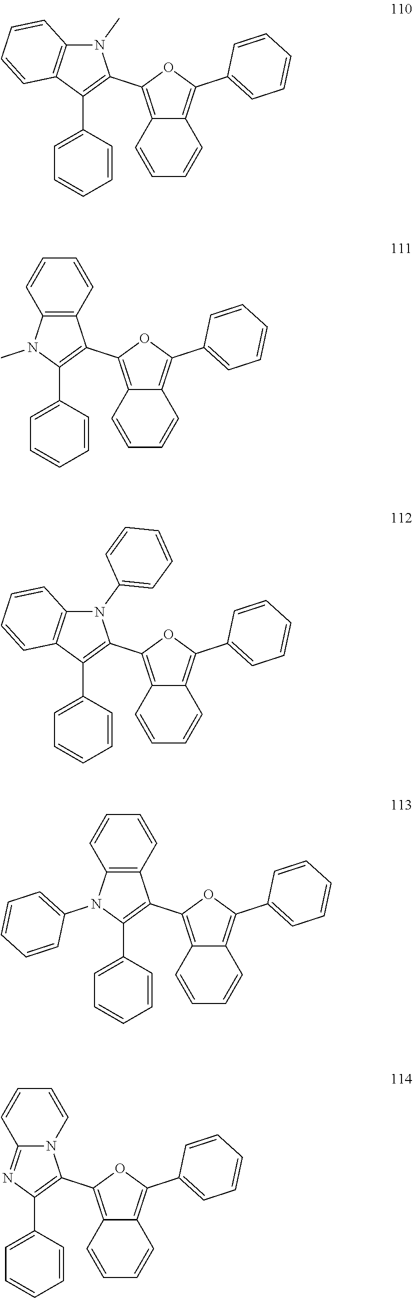 [SMILES: Cn1c(-c2oc(-c3ccccc3)c3ccccc23)c(-c2ccccc2)c2ccccc21 ; Cn1c(-c2ccccc2)c(-c2oc(-c3ccccc3)c3ccccc23)c2ccccc21 ; c1ccc(-c2oc(-c3c(-c4ccccc4)c4ccccc4n3-c3ccccc3)c3ccccc23)cc1 ; c1ccc(-c2oc(-c3c(-c4ccccc4)n(-c4ccccc4)c4ccccc34)c3ccccc23)cc1 ; c1ccc(-c2nc3ccccn3c2-c2oc(-c3ccccc3)c3ccccc23)cc1]

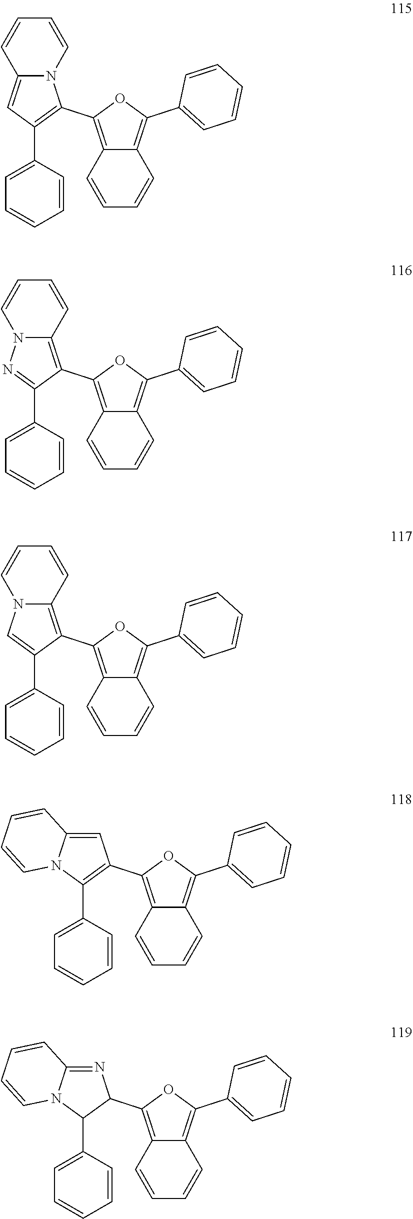 [SMILES: c1ccc(-c2cc3ccccn3c2-c2oc(-c3ccccc3)c3ccccc23)cc1 ; c1ccc(-c2nn3ccccc3c2-c2oc(-c3ccccc3)c3ccccc23)cc1 ; c1ccc(-c2cn3ccccc3c2-c2oc(-c3ccccc3)c3ccccc23)cc1 ; c1ccc(-c2oc(-c3cc4ccccn4c3-c3ccccc3)c3ccccc23)cc1 ; C1=CC2=NC(c3oc(-c4ccccc4)c4ccccc34)C(c3ccccc3)N2C=C1]

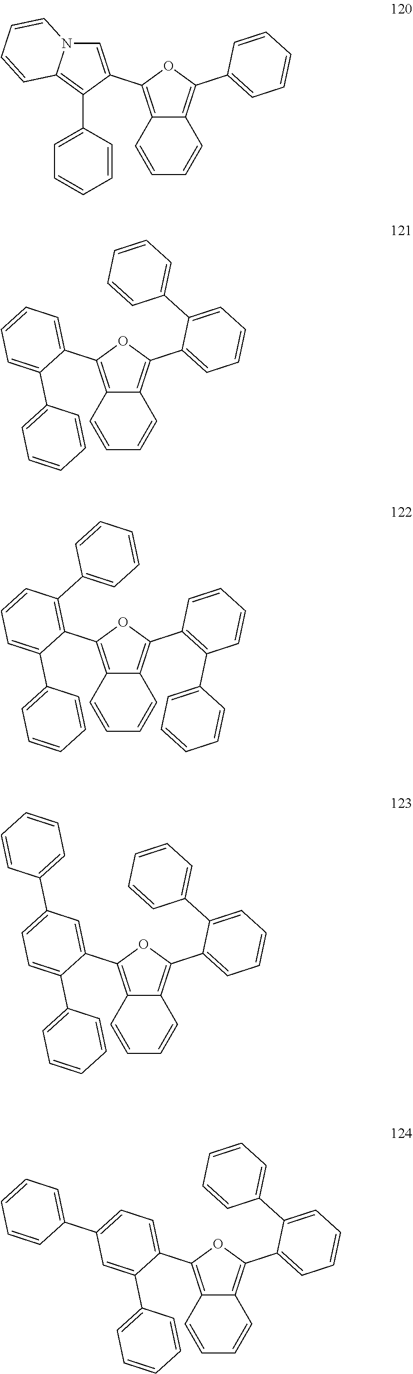 [SMILES: c1ccc(-c2oc(-c3cn4ccccc4c3-c3ccccc3)c3ccccc23)cc1 ; c1ccc(-c2ccccc2-c2oc(-c3ccccc3-c3ccccc3)c3ccccc23)cc1 ; c1ccc(-c2ccccc2-c2oc(-c3c(-c4ccccc4)cccc3-c3ccccc3)c3ccccc23)cc1 ; c1ccc(-c2ccc(-c3ccccc3)c(-c3oc(-c4ccccc4-c4ccccc4)c4ccccc34)c2)cc1 ; c1ccc(-c2ccc(-c3oc(-c4ccccc4-c4ccccc4)c4ccccc34)c(-c3ccccc3)c2)cc1]

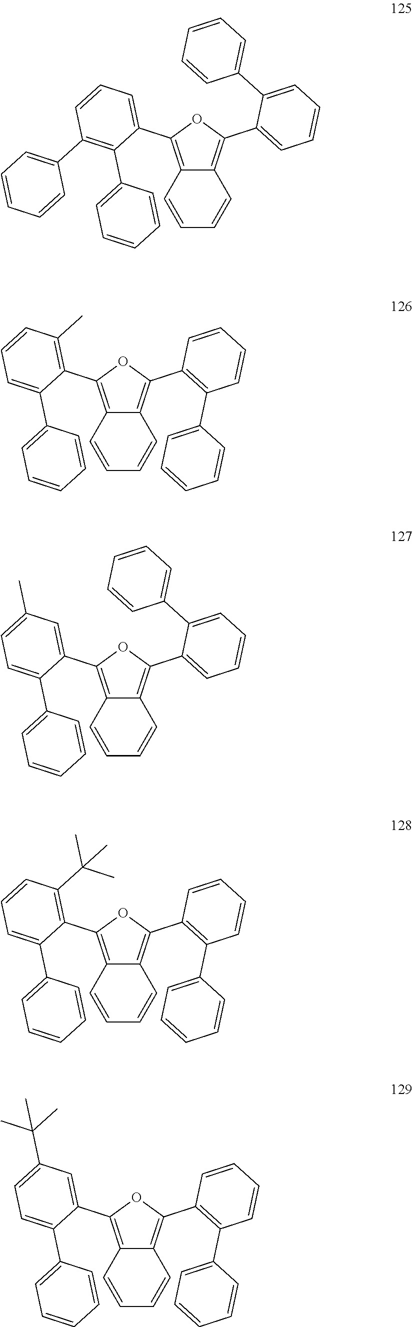 [SMILES: c1ccc(-c2ccccc2-c2oc(-c3cccc(-c4ccccc4)c3-c3ccccc3)c3ccccc23)cc1 ; Cc1cccc(-c2ccccc2)c1-c1oc(-c2ccccc2-c2ccccc2)c2ccccc12 ; Cc1ccc(-c2ccccc2)c(-c2oc(-c3ccccc3-c3ccccc3)c3ccccc23)c1 ; CC(C)(C)c1cccc(-c2ccccc2)c1-c1oc(-c2ccccc2-c2ccccc2)c2ccccc12 ; CC(C)(C)c1ccc(-c2ccccc2)c(-c2oc(-c3ccccc3-c3ccccc3)c3ccccc23)c1]

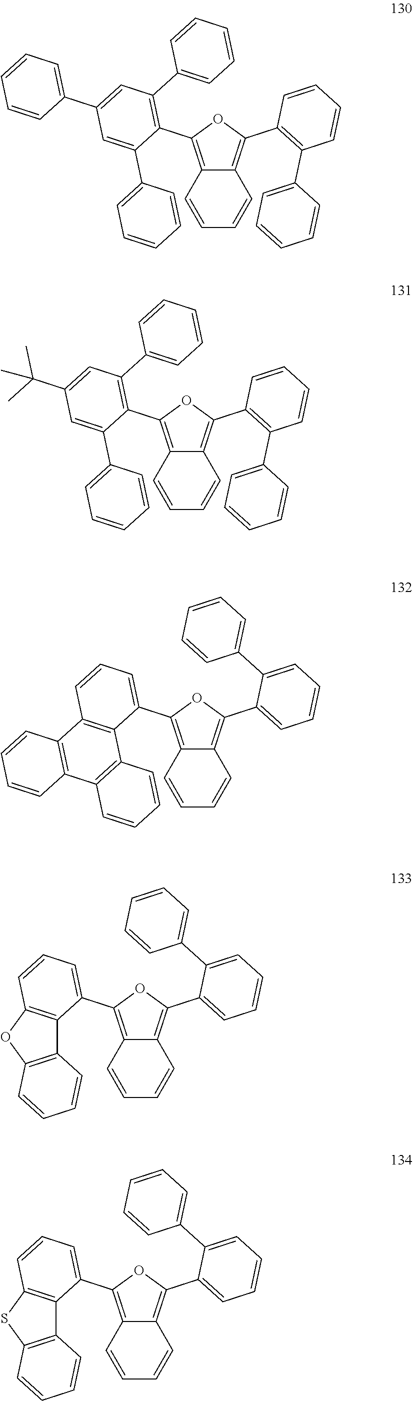 [SMILES: c1ccc(-c2cc(-c3ccccc3)c(-c3oc(-c4ccccc4-c4ccccc4)c4ccccc34)c(-c3ccccc3)c2)cc1 ; CC(C)(C)c1cc(-c2ccccc2)c(-c2oc(-c3ccccc3-c3ccccc3)c3ccccc23)c(-c2ccccc2)c1 ; c1ccc(-c2ccccc2-c2oc(-c3cccc4c5ccccc5c5ccccc5c34)c3ccccc23)cc1 ; c1ccc(-c2ccccc2-c2oc(-c3cccc4oc5ccccc5c34)c3ccccc23)cc1 ; c1ccc(-c2ccccc2-c2oc(-c3cccc4sc5ccccc5c34)c3ccccc23)cc1]

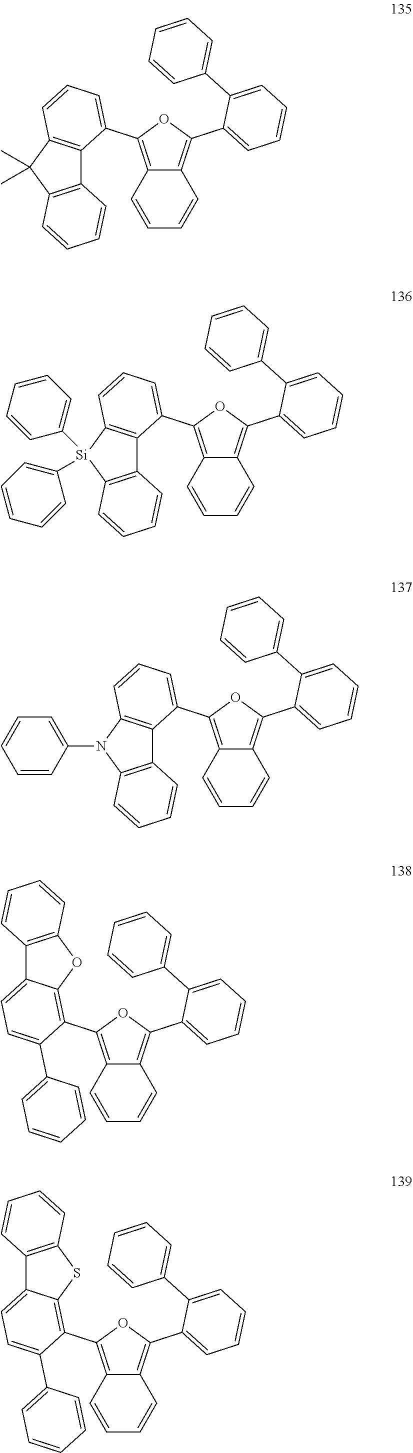 [SMILES: CC1(C)c2ccccc2-c2c(-c3oc(-c4ccccc4-c4ccccc4)c4ccccc34)cccc21 ; c1ccc(-c2ccccc2-c2oc(-c3cccc4c3-c3ccccc3[Si]4(c3ccccc3)c3ccccc3)c3ccccc23)cc1 ; c1ccc(-c2ccccc2-c2oc(-c3cccc4c3c3ccccc3n4-c3ccccc3)c3ccccc23)cc1 ; c1ccc(-c2ccccc2-c2oc(-c3c(-c4ccccc4)ccc4c3oc3ccccc34)c3ccccc23)cc1 ; c1ccc(-c2ccccc2-c2oc(-c3c(-c4ccccc4)ccc4c3sc3ccccc34)c3ccccc23)cc1]

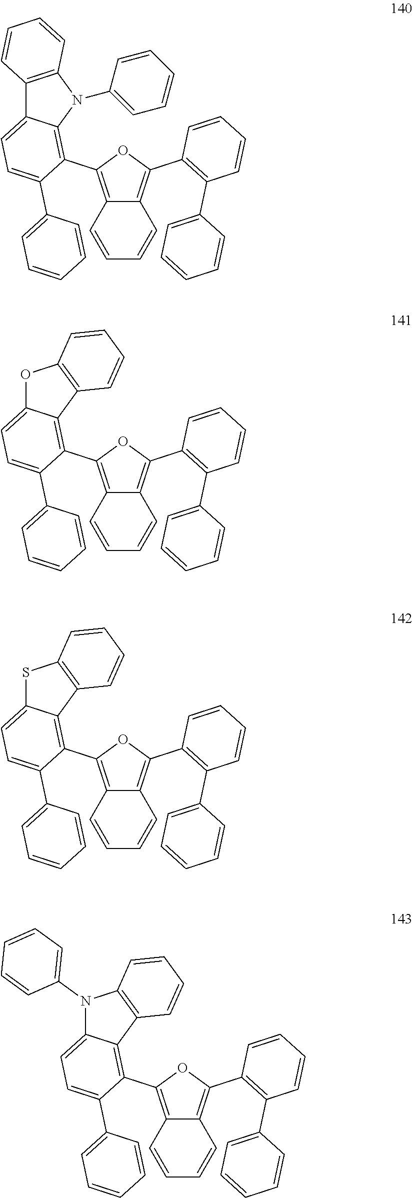 [SMILES: c1ccc(-c2ccccc2-c2oc(-c3c(-c4ccccc4)ccc4c5ccccc5n(-c5ccccc5)c34)c3ccccc23)cc1 ; c1ccc(-c2ccccc2-c2oc(-c3c(-c4ccccc4)ccc4oc5ccccc5c34)c3ccccc23)cc1 ; c1ccc(-c2ccccc2-c2oc(-c3c(-c4ccccc4)ccc4sc5ccccc5c34)c3ccccc23)cc1 ; c1ccc(-c2ccccc2-c2oc(-c3c(-c4ccccc4)ccc4c3c3ccccc3n4-c3ccccc3)c3ccccc23)cc1]

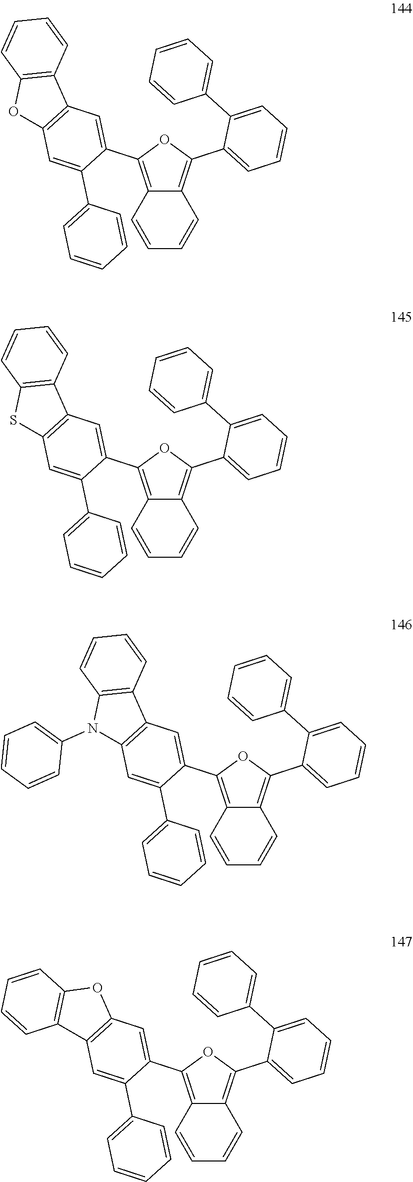 [SMILES: c1ccc(-c2ccccc2-c2oc(-c3cc4c(cc3-c3ccccc3)oc3ccccc34)c3ccccc23)cc1 ; c1ccc(-c2ccccc2-c2oc(-c3cc4c(cc3-c3ccccc3)sc3ccccc34)c3ccccc23)cc1 ; c1ccc(-c2ccccc2-c2oc(-c3cc4c5ccccc5n(-c5ccccc5)c4cc3-c3ccccc3)c3ccccc23)cc1 ; c1ccc(-c2ccccc2-c2oc(-c3cc4oc5ccccc5c4cc3-c3ccccc3)c3ccccc23)cc1]

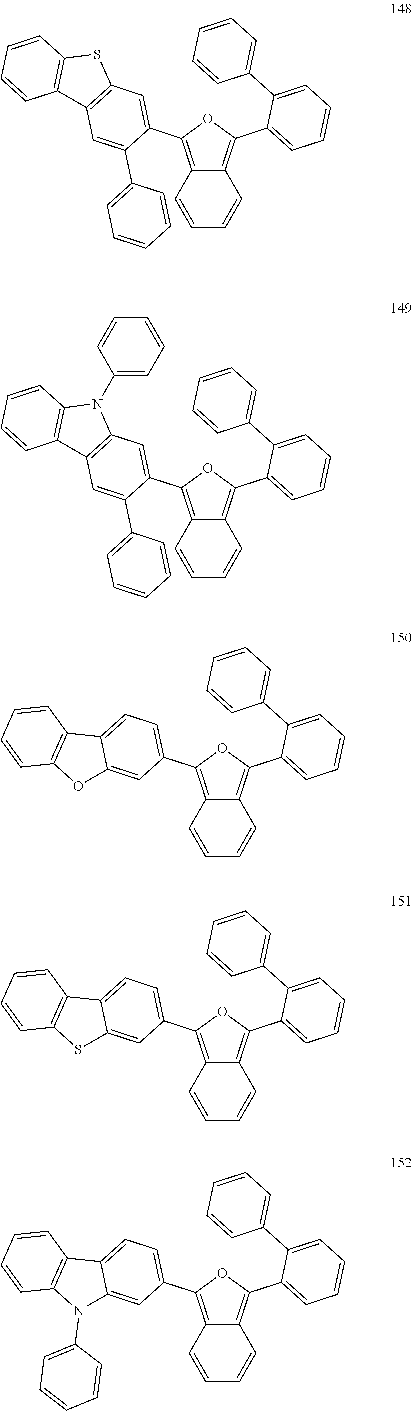 [SMILES: c1ccc(-c2ccccc2-c2oc(-c3cc4sc5ccccc5c4cc3-c3ccccc3)c3ccccc23)cc1 ; c1ccc(-c2ccccc2-c2oc(-c3cc4c(cc3-c3ccccc3)c3ccccc3n4-c3ccccc3)c3ccccc23)cc1 ; c1ccc(-c2ccccc2-c2oc(-c3ccc4c(c3)oc3ccccc34)c3ccccc23)cc1 ; c1ccc(-c2ccccc2-c2oc(-c3ccc4c(c3)sc3ccccc34)c3ccccc23)cc1 ; c1ccc(-c2ccccc2-c2oc(-c3ccc4c5ccccc5n(-c5ccccc5)c4c3)c3ccccc23)cc1]

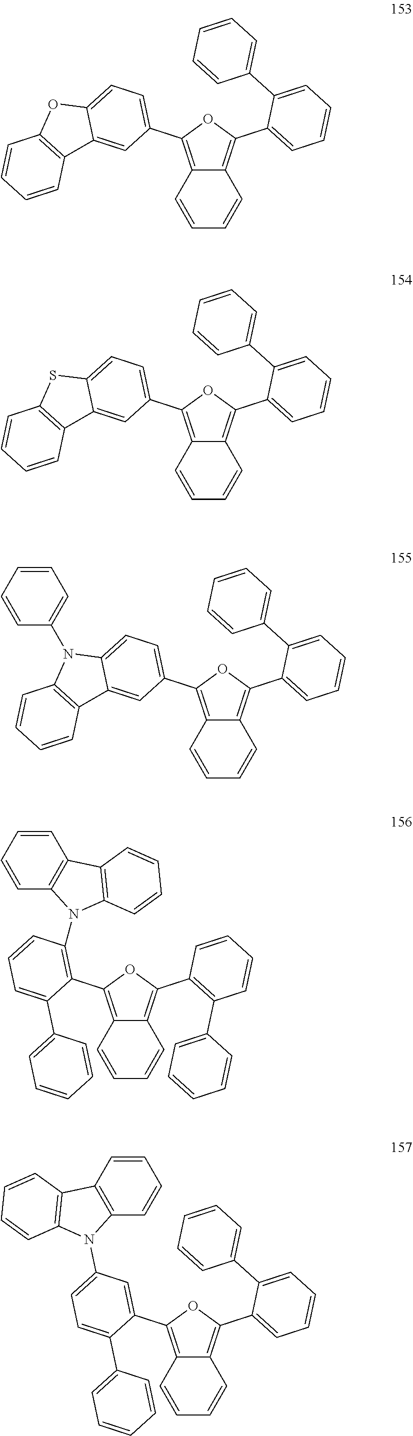 [SMILES: c1ccc(-c2ccccc2-c2oc(-c3ccc4oc5ccccc5c4c3)c3ccccc23)cc1 ; c1ccc(-c2ccccc2-c2oc(-c3ccc4sc5ccccc5c4c3)c3ccccc23)cc1 ; c1ccc(-c2ccccc2-c2oc(-c3ccc4c(c3)c3ccccc3n4-c3ccccc3)c3ccccc23)cc1 ; c1ccc(-c2ccccc2-c2oc(-c3c(-c4ccccc4)cccc3-n3c4ccccc4c4ccccc43)c3ccccc23)cc1 ; c1ccc(-c2ccccc2-c2oc(-c3cc(-n4c5ccccc5c5ccccc54)ccc3-c3ccccc3)c3ccccc23)cc1]

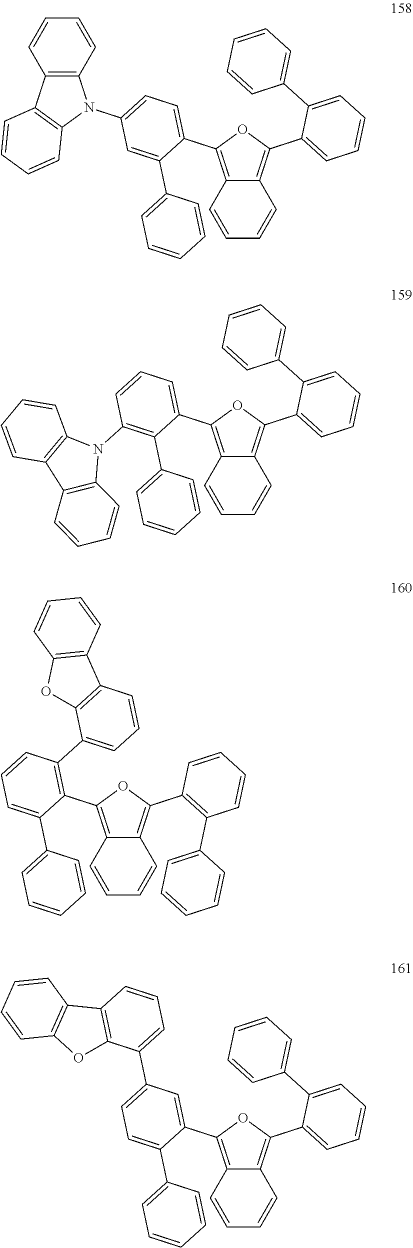 [SMILES: c1ccc(-c2ccccc2-c2oc(-c3ccc(-n4c5ccccc5c5ccccc54)cc3-c3ccccc3)c3ccccc23)cc1 ; c1ccc(-c2ccccc2-c2oc(-c3cccc(-n4c5ccccc5c5ccccc54)c3-c3ccccc3)c3ccccc23)cc1 ; c1ccc(-c2ccccc2-c2oc(-c3c(-c4ccccc4)cccc3-c3cccc4c3oc3ccccc34)c3ccccc23)cc1 ; c1ccc(-c2ccccc2-c2oc(-c3cc(-c4cccc5c4oc4ccccc45)ccc3-c3ccccc3)c3ccccc23)cc1]

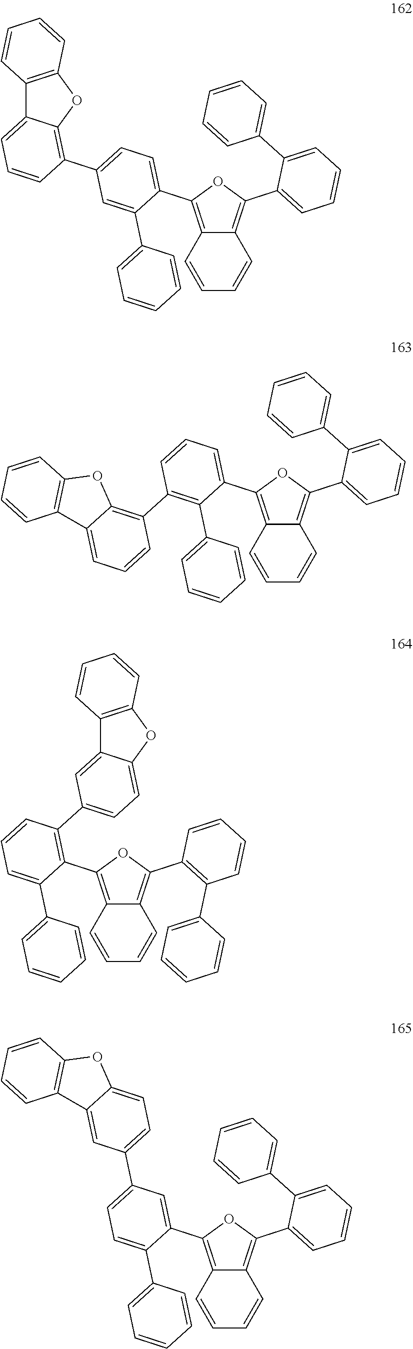 [SMILES: c1ccc(-c2ccccc2-c2oc(-c3ccc(-c4cccc5c4oc4ccccc45)cc3-c3ccccc3)c3ccccc23)cc1 ; c1ccc(-c2ccccc2-c2oc(-c3cccc(-c4cccc5c4oc4ccccc45)c3-c3ccccc3)c3ccccc23)cc1 ; c1ccc(-c2ccccc2-c2oc(-c3c(-c4ccccc4)cccc3-c3ccc4oc5ccccc5c4c3)c3ccccc23)cc1 ; c1ccc(-c2ccccc2-c2oc(-c3cc(-c4ccc5oc6ccccc6c5c4)ccc3-c3ccccc3)c3ccccc23)cc1]

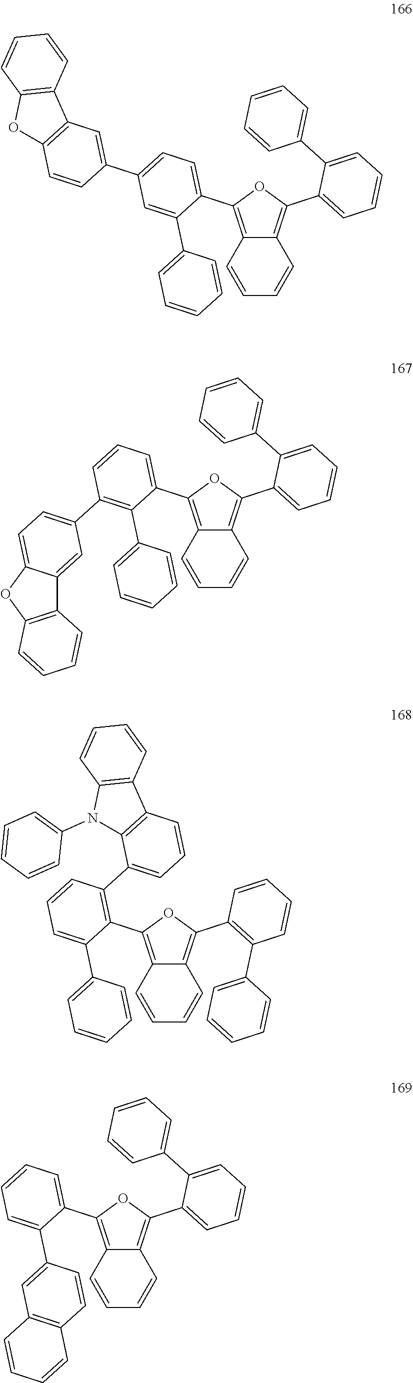 [SMILES: c1ccc(-c2ccccc2-c2oc(-c3ccc(-c4ccc5oc6ccccc6c5c4)cc3-c3ccccc3)c3ccccc23)cc1 ; c1ccc(-c2ccccc2-c2oc(-c3cccc(-c4ccc5oc6ccccc6c5c4)c3-c3ccccc3)c3ccccc23)cc1 ; c1ccc(-c2ccccc2-c2oc(-c3c(-c4ccccc4)cccc3-c3cccc4c5ccccc5n(-c5ccccc5)c34)c3ccccc23)cc1 ; c1ccc(-c2ccccc2-c2oc(-c3ccccc3-c3ccc4ccccc4c3)c3ccccc23)cc1]

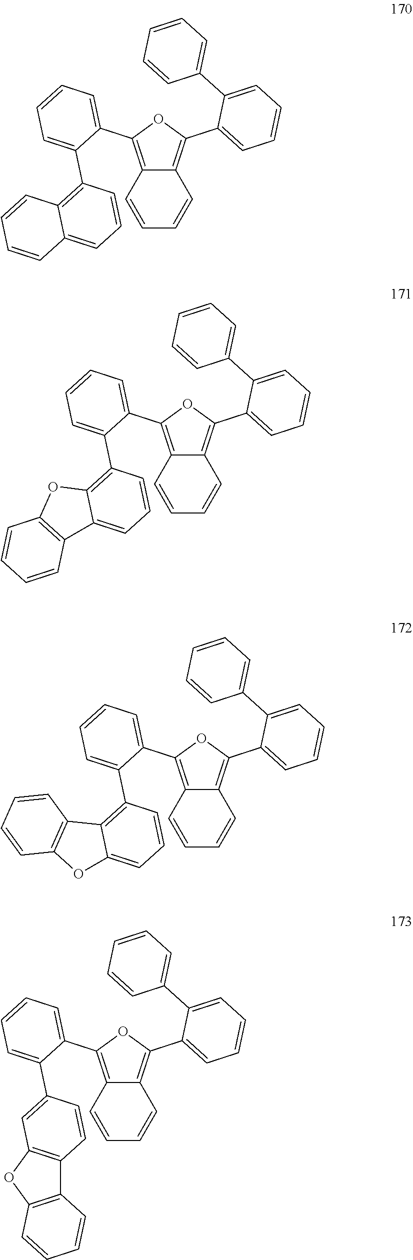 [SMILES: c1ccc(-c2ccccc2-c2oc(-c3ccccc3-c3cccc4ccccc34)c3ccccc23)cc1 ; c1ccc(-c2ccccc2-c2oc(-c3ccccc3-c3cccc4c3oc3ccccc34)c3ccccc23)cc1 ; c1ccc(-c2ccccc2-c2oc(-c3ccccc3-c3cccc4oc5ccccc5c34)c3ccccc23)cc1 ; c1ccc(-c2ccccc2-c2oc(-c3ccccc3-c3ccc4c(c3)oc3ccccc34)c3ccccc23)cc1]

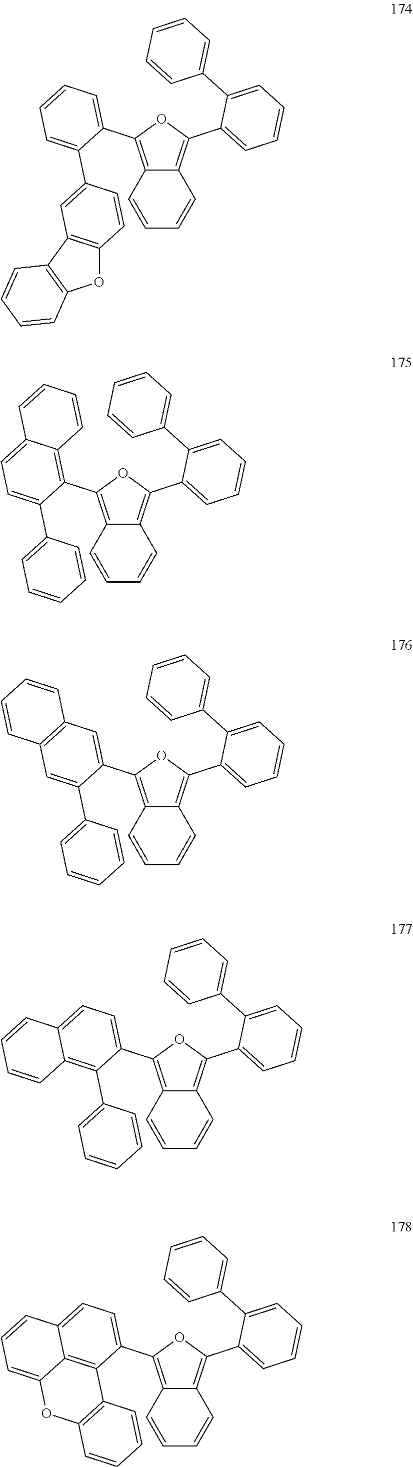 [SMILES: c1ccc(-c2ccccc2-c2oc(-c3ccccc3-c3ccc4oc5ccccc5c4c3)c3ccccc23)cc1 ; c1ccc(-c2ccccc2-c2oc(-c3c(-c4ccccc4)ccc4ccccc34)c3ccccc23)cc1 ; c1ccc(-c2ccccc2-c2oc(-c3cc4ccccc4cc3-c3ccccc3)c3ccccc23)cc1 ; c1ccc(-c2ccccc2-c2oc(-c3ccc4ccccc4c3-c3ccccc3)c3ccccc23)cc1 ; c1ccc(-c2ccccc2-c2oc(-c3ccc4cccc5c4c3-c3ccccc3O5)c3ccccc23)cc1]

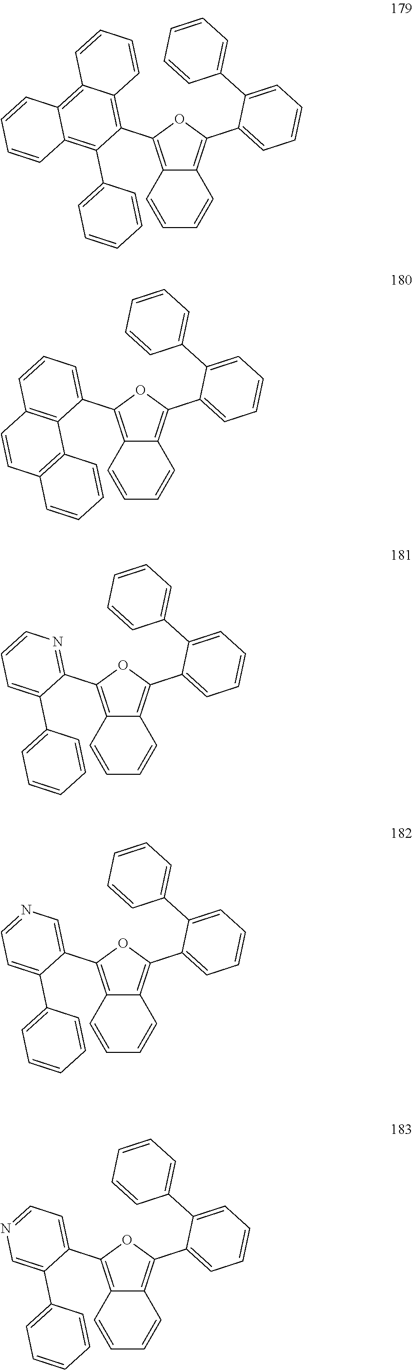 [SMILES: c1ccc(-c2ccccc2-c2oc(-c3c(-c4ccccc4)c4ccccc4c4ccccc34)c3ccccc23)cc1 ; c1ccc(-c2ccccc2-c2oc(-c3cccc4ccc5ccccc5c34)c3ccccc23)cc1 ; c1ccc(-c2ccccc2-c2oc(-c3ncccc3-c3ccccc3)c3ccccc23)cc1 ; c1ccc(-c2ccccc2-c2oc(-c3cnccc3-c3ccccc3)c3ccccc23)cc1 ; c1ccc(-c2ccccc2-c2oc(-c3ccncc3-c3ccccc3)c3ccccc23)cc1]

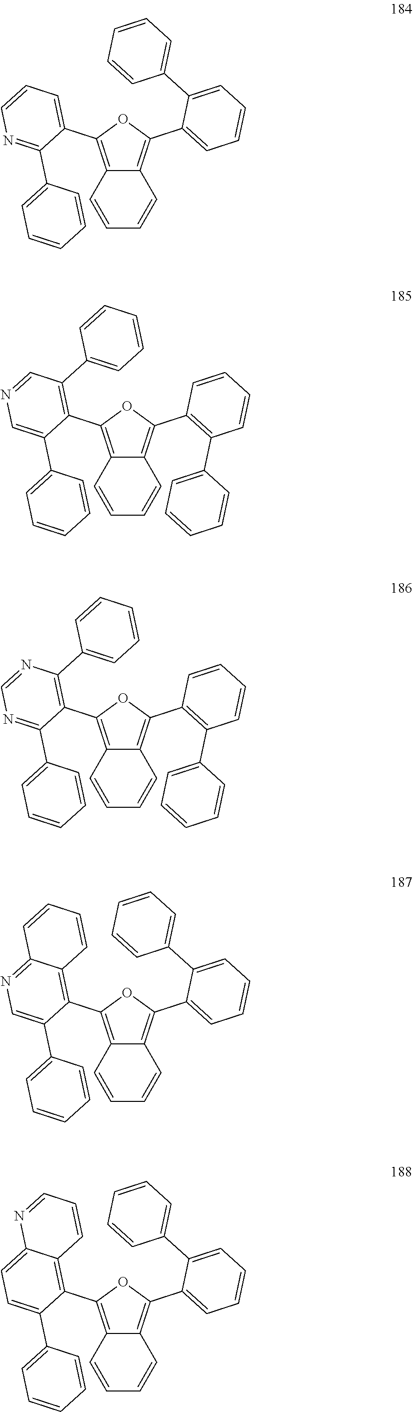 [SMILES: c1ccc(-c2ccccc2-c2oc(-c3cccnc3-c3ccccc3)c3ccccc23)cc1 ; c1ccc(-c2ccccc2-c2oc(-c3c(-c4ccccc4)cncc3-c3ccccc3)c3ccccc23)cc1 ; c1ccc(-c2ccccc2-c2oc(-c3c(-c4ccccc4)ncnc3-c3ccccc3)c3ccccc23)cc1 ; c1ccc(-c2ccccc2-c2oc(-c3c(-c4ccccc4)cnc4ccccc34)c3ccccc23)cc1 ; c1ccc(-c2ccccc2-c2oc(-c3c(-c4ccccc4)ccc4ncccc34)c3ccccc23)cc1]

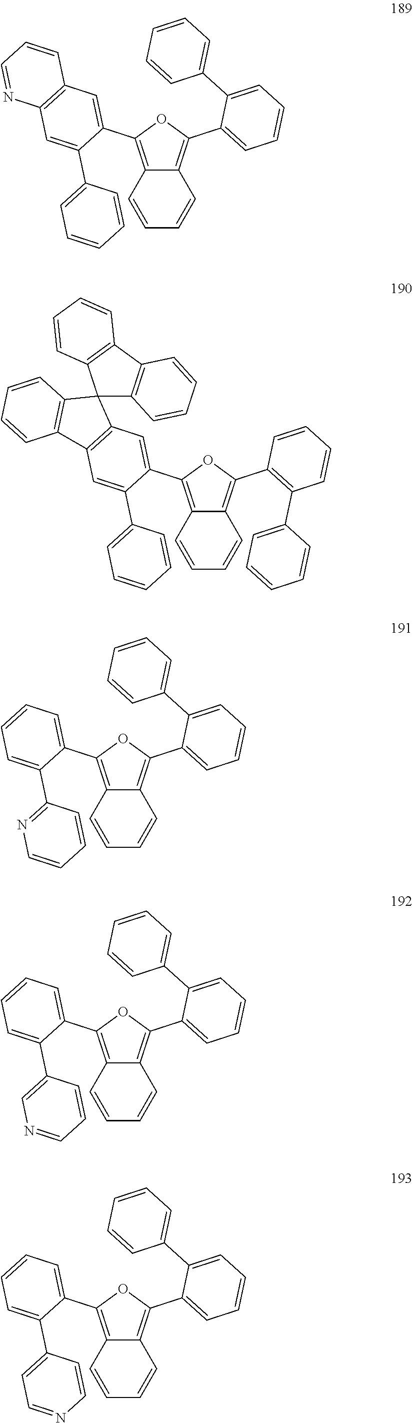 [SMILES: c1ccc(-c2ccccc2-c2oc(-c3cc4cccnc4cc3-c3ccccc3)c3ccccc23)cc1 ; c1ccc(-c2ccccc2-c2oc(-c3cc4c(cc3-c3ccccc3)-c3ccccc3C43c4ccccc4-c4ccccc43)c3ccccc23)cc1 ; c1ccc(-c2ccccc2-c2oc(-c3ccccc3-c3ccccn3)c3ccccc23)cc1 ; c1ccc(-c2ccccc2-c2oc(-c3ccccc3-c3cccnc3)c3ccccc23)cc1 ; c1ccc(-c2ccccc2-c2oc(-c3ccccc3-c3ccncc3)c3ccccc23)cc1]

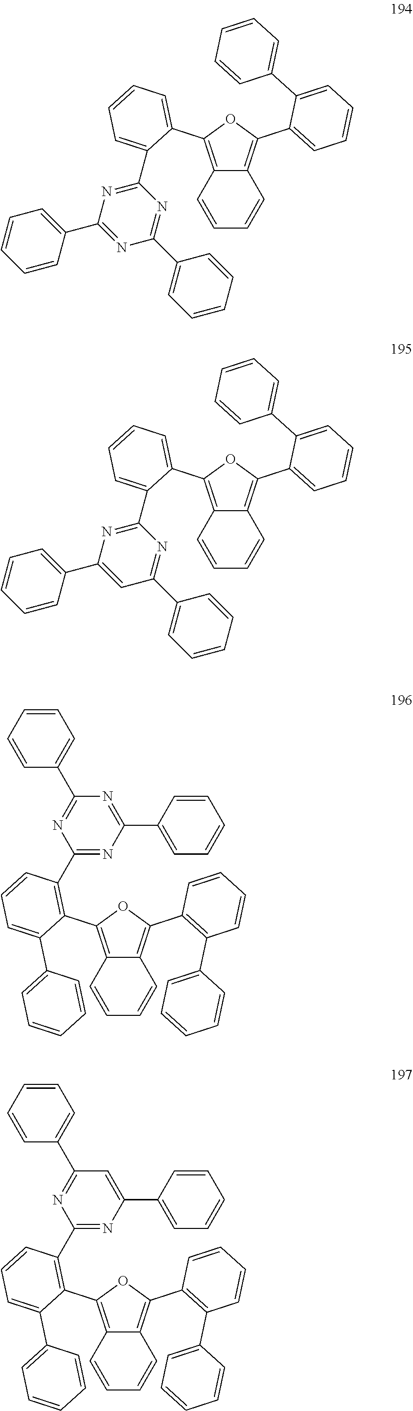 [SMILES: c1ccc(-c2nc(-c3ccccc3)nc(-c3ccccc3-c3oc(-c4ccccc4-c4ccccc4)c4ccccc34)n2)cc1 ; c1ccc(-c2cc(-c3ccccc3)nc(-c3ccccc3-c3oc(-c4ccccc4-c4ccccc4)c4ccccc34)n2)cc1 ; c1ccc(-c2nc(-c3ccccc3)nc(-c3cccc(-c4ccccc4)c3-c3oc(-c4ccccc4-c4ccccc4)c4ccccc34)n2)cc1 ; c1ccc(-c2cc(-c3ccccc3)nc(-c3cccc(-c4ccccc4)c3-c3oc(-c4ccccc4-c4ccccc4)c4ccccc34)n2)cc1]

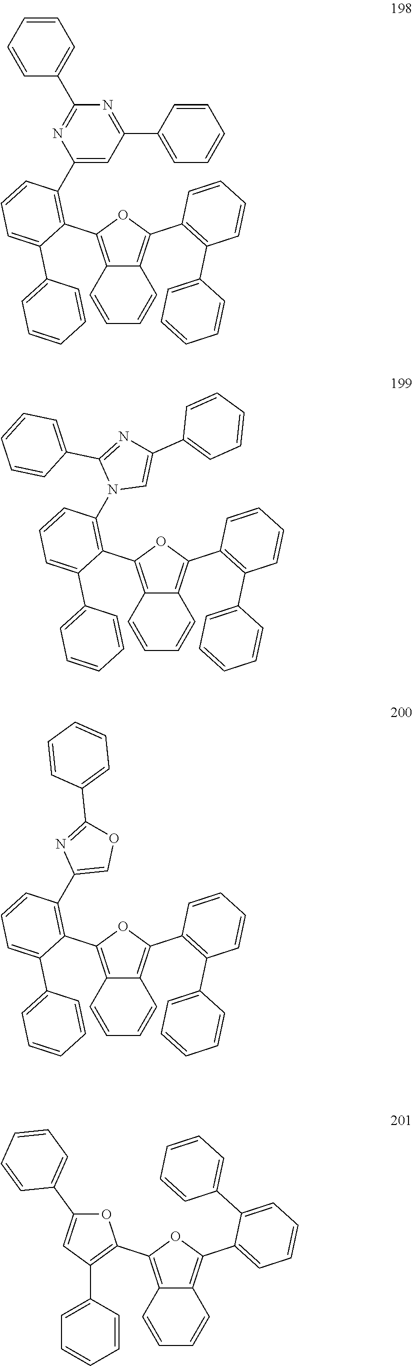 [SMILES: c1ccc(-c2cc(-c3cccc(-c4ccccc4)c3-c3oc(-c4ccccc4-c4ccccc4)c4ccccc34)nc(-c3ccccc3)n2)cc1 ; c1ccc(-c2cn(-c3cccc(-c4ccccc4)c3-c3oc(-c4ccccc4-c4ccccc4)c4ccccc34)c(-c3ccccc3)n2)cc1 ; c1ccc(-c2nc(-c3cccc(-c4ccccc4)c3-c3oc(-c4ccccc4-c4ccccc4)c4ccccc34)co2)cc1 ; c1ccc(-c2cc(-c3ccccc3)c(-c3oc(-c4ccccc4-c4ccccc4)c4ccccc34)o2)cc1]

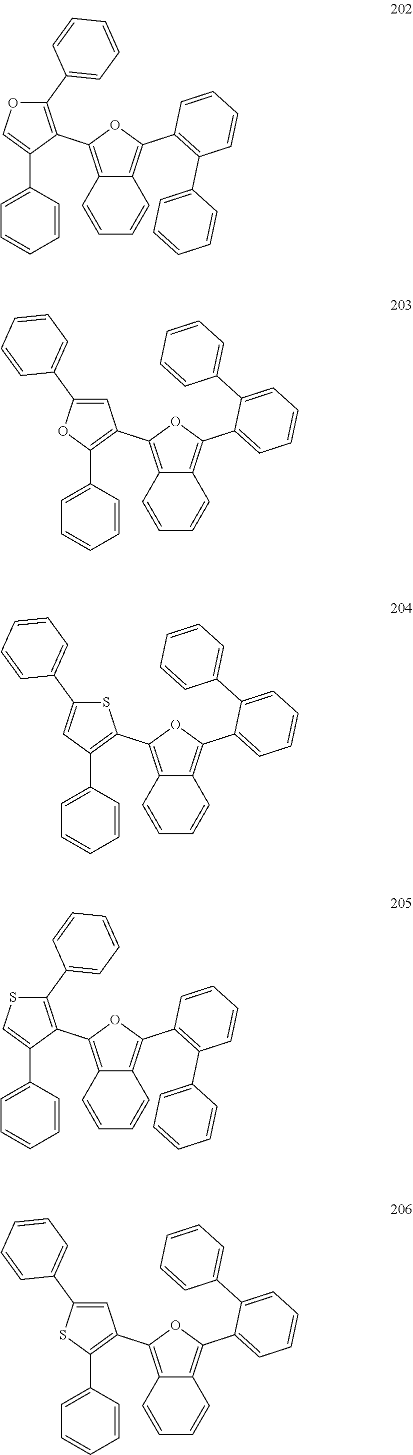 [SMILES: c1ccc(-c2ccccc2-c2oc(-c3c(-c4ccccc4)coc3-c3ccccc3)c3ccccc23)cc1 ; c1ccc(-c2cc(-c3oc(-c4ccccc4-c4ccccc4)c4ccccc34)c(-c3ccccc3)o2)cc1 ; c1ccc(-c2cc(-c3ccccc3)c(-c3oc(-c4ccccc4-c4ccccc4)c4ccccc34)s2)cc1 ; c1ccc(-c2ccccc2-c2oc(-c3c(-c4ccccc4)csc3-c3ccccc3)c3ccccc23)cc1 ; c1ccc(-c2cc(-c3oc(-c4ccccc4-c4ccccc4)c4ccccc34)c(-c3ccccc3)s2)cc1]

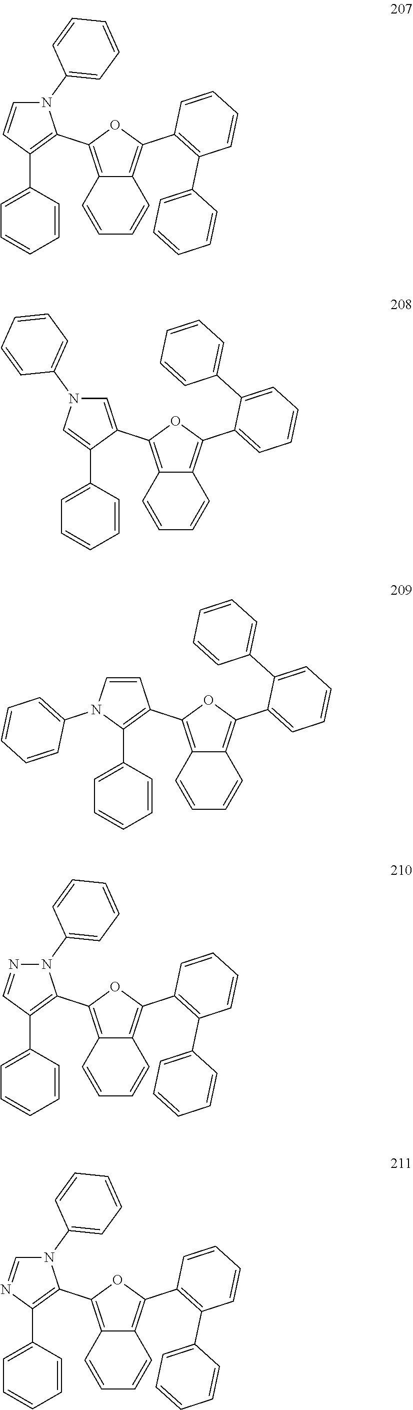 [SMILES: c1ccc(-c2ccccc2-c2oc(-c3c(-c4ccccc4)ccn3-c3ccccc3)c3ccccc23)cc1 ; c1ccc(-c2ccccc2-c2oc(-c3cn(-c4ccccc4)cc3-c3ccccc3)c3ccccc23)cc1 ; c1ccc(-c2ccccc2-c2oc(-c3ccn(-c4ccccc4)c3-c3ccccc3)c3ccccc23)cc1 ; c1ccc(-c2ccccc2-c2oc(-c3c(-c4ccccc4)cnn3-c3ccccc3)c3ccccc23)cc1 ; c1ccc(-c2ccccc2-c2oc(-c3c(-c4ccccc4)ncn3-c3ccccc3)c3ccccc23)cc1]

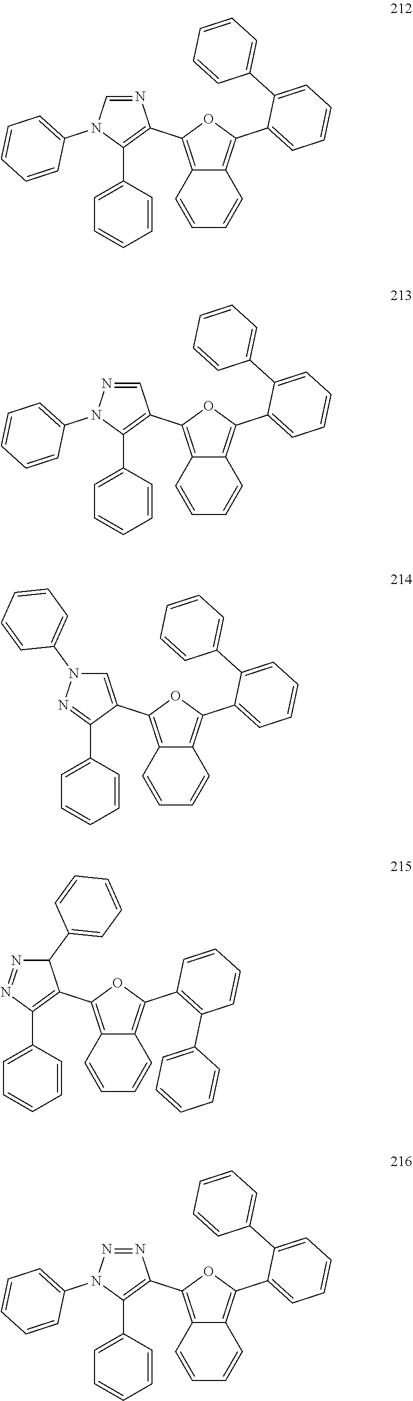 [SMILES: c1ccc(-c2ccccc2-c2oc(-c3ncn(-c4ccccc4)c3-c3ccccc3)c3ccccc23)cc1 ; c1ccc(-c2ccccc2-c2oc(-c3cnn(-c4ccccc4)c3-c3ccccc3)c3ccccc23)cc1 ; c1ccc(-c2ccccc2-c2oc(-c3cn(-c4ccccc4)nc3-c3ccccc3)c3ccccc23)cc1 ; c1ccc(C2=C(c3oc(-c4ccccc4-c4ccccc4)c4ccccc34)C(c3ccccc3)N=N2)cc1 ; c1ccc(-c2ccccc2-c2oc(-c3nnn(-c4ccccc4)c3-c3ccccc3)c3ccccc23)cc1]

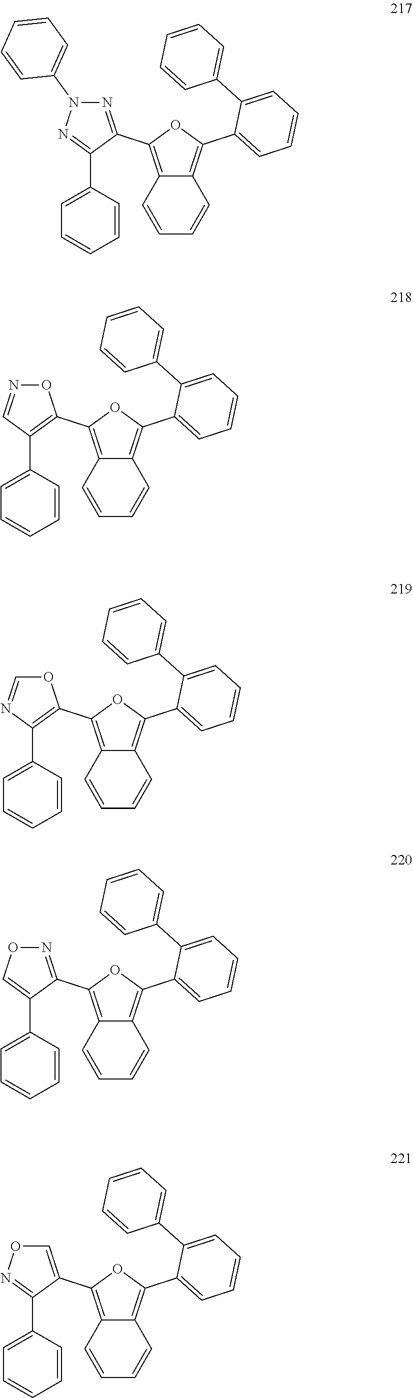 [SMILES: c1ccc(-c2ccccc2-c2oc(-c3nn(-c4ccccc4)nc3-c3ccccc3)c3ccccc23)cc1 ; c1ccc(-c2ccccc2-c2oc(-c3oncc3-c3ccccc3)c3ccccc23)cc1 ; c1ccc(-c2ccccc2-c2oc(-c3ocnc3-c3ccccc3)c3ccccc23)cc1 ; c1ccc(-c2ccccc2-c2oc(-c3nocc3-c3ccccc3)c3ccccc23)cc1 ; c1ccc(-c2ccccc2-c2oc(-c3conc3-c3ccccc3)c3ccccc23)cc1]

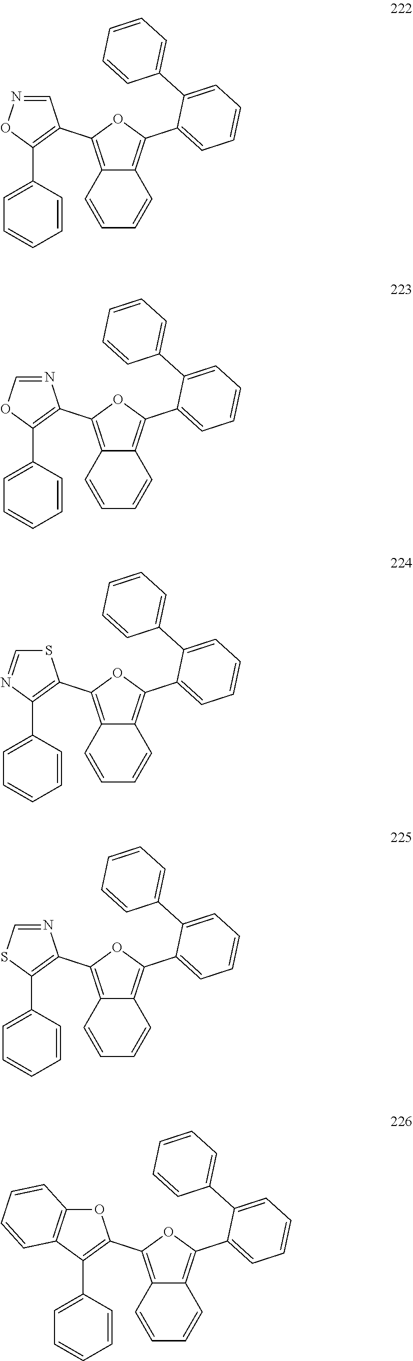 [SMILES: c1ccc(-c2ccccc2-c2oc(-c3cnoc3-c3ccccc3)c3ccccc23)cc1 ; c1ccc(-c2ccccc2-c2oc(-c3ncoc3-c3ccccc3)c3ccccc23)cc1 ; c1ccc(-c2ccccc2-c2oc(-c3scnc3-c3ccccc3)c3ccccc23)cc1 ; c1ccc(-c2ccccc2-c2oc(-c3ncsc3-c3ccccc3)c3ccccc23)cc1 ; c1ccc(-c2ccccc2-c2oc(-c3oc4ccccc4c3-c3ccccc3)c3ccccc23)cc1]

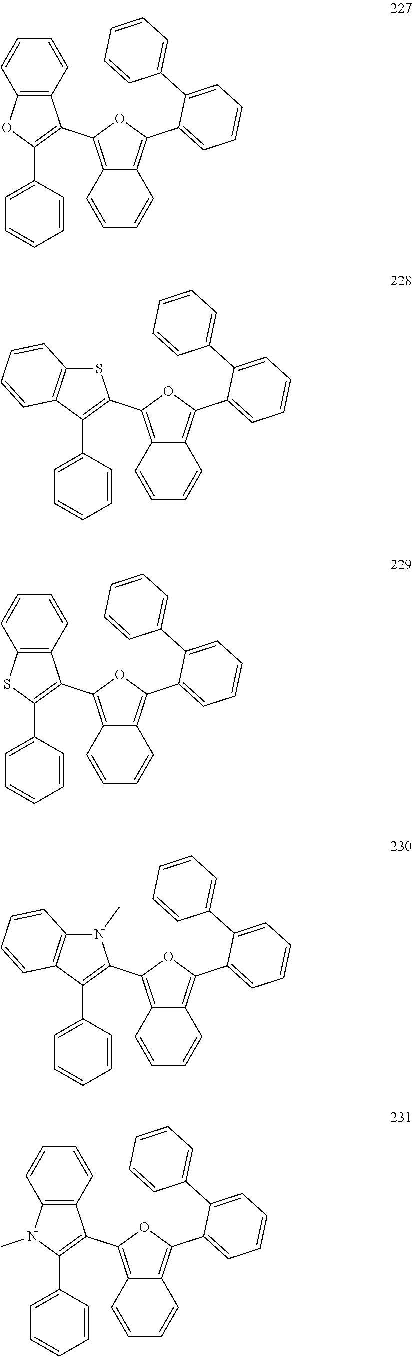 [SMILES: c1ccc(-c2ccccc2-c2oc(-c3c(-c4ccccc4)oc4ccccc34)c3ccccc23)cc1 ; c1ccc(-c2ccccc2-c2oc(-c3sc4ccccc4c3-c3ccccc3)c3ccccc23)cc1 ; c1ccc(-c2ccccc2-c2oc(-c3c(-c4ccccc4)sc4ccccc34)c3ccccc23)cc1 ; Cn1c(-c2oc(-c3ccccc3-c3ccccc3)c3ccccc23)c(-c2ccccc2)c2ccccc21 ; Cn1c(-c2ccccc2)c(-c2oc(-c3ccccc3-c3ccccc3)c3ccccc23)c2ccccc21]

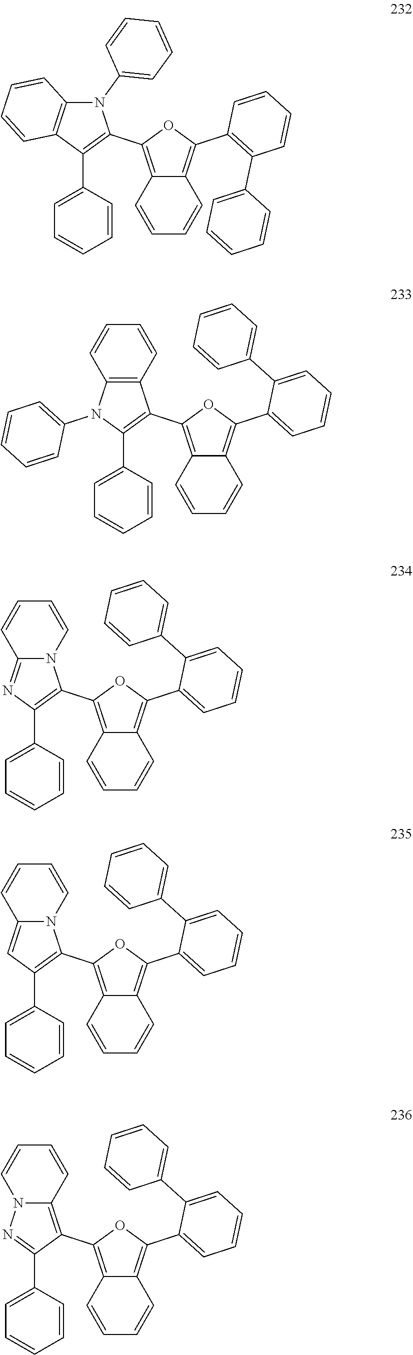 [SMILES: c1ccc(-c2ccccc2-c2oc(-c3c(-c4ccccc4)c4ccccc4n3-c3ccccc3)c3ccccc23)cc1 ; c1ccc(-c2ccccc2-c2oc(-c3c(-c4ccccc4)n(-c4ccccc4)c4ccccc34)c3ccccc23)cc1 ; c1ccc(-c2ccccc2-c2oc(-c3c(-c4ccccc4)nc4ccccn34)c3ccccc23)cc1 ; c1ccc(-c2ccccc2-c2oc(-c3c(-c4ccccc4)cc4ccccn34)c3ccccc23)cc1 ; c1ccc(-c2ccccc2-c2oc(-c3c(-c4ccccc4)nn4ccccc34)c3ccccc23)cc1]

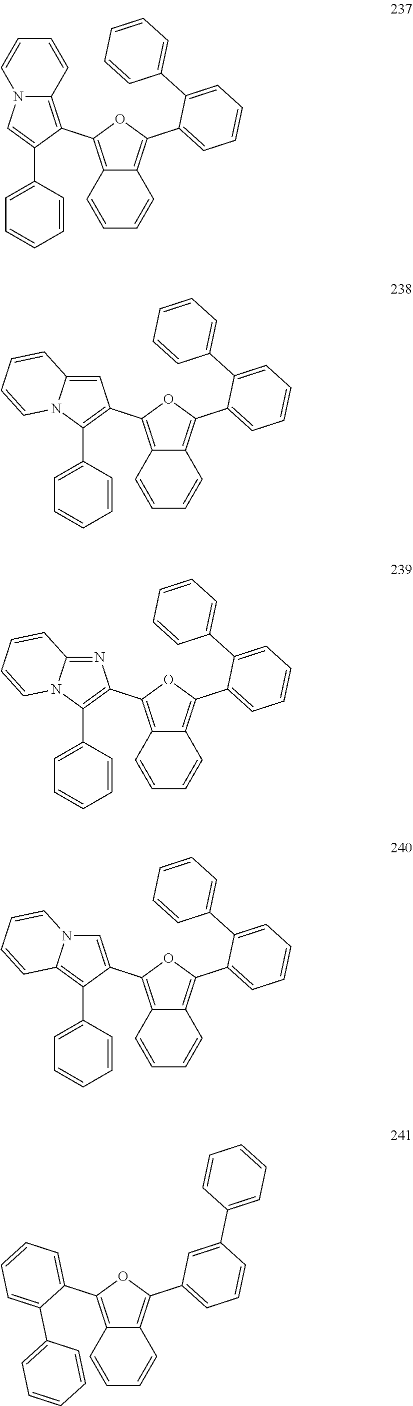 [SMILES: c1ccc(-c2ccccc2-c2oc(-c3c(-c4ccccc4)cn4ccccc34)c3ccccc23)cc1 ; c1ccc(-c2ccccc2-c2oc(-c3cc4ccccn4c3-c3ccccc3)c3ccccc23)cc1 ; c1ccc(-c2ccccc2-c2oc(-c3nc4ccccn4c3-c3ccccc3)c3ccccc23)cc1 ; c1ccc(-c2ccccc2-c2oc(-c3cn4ccccc4c3-c3ccccc3)c3ccccc23)cc1 ; c1ccc(-c2cccc(-c3oc(-c4ccccc4-c4ccccc4)c4ccccc34)c2)cc1]

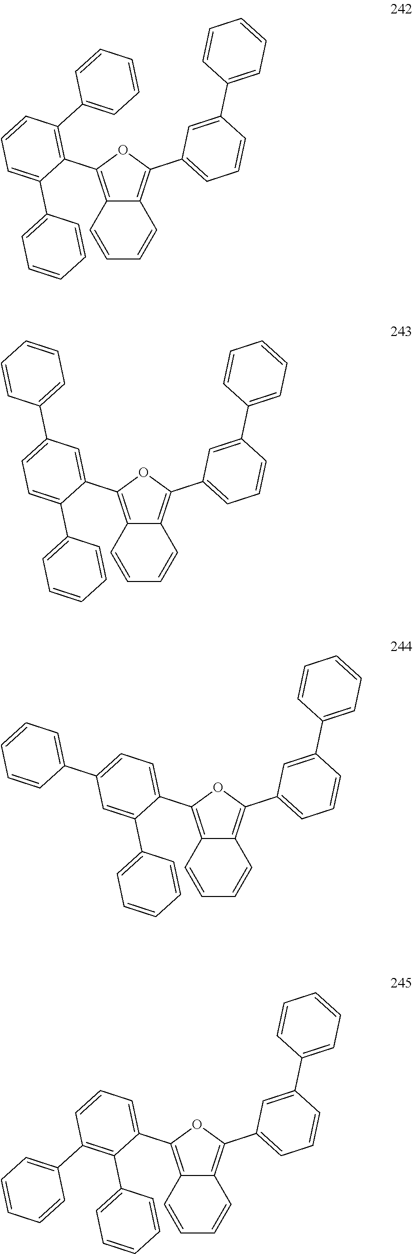 [SMILES: c1ccc(-c2cccc(-c3oc(-c4c(-c5ccccc5)cccc4-c4ccccc4)c4ccccc34)c2)cc1 ; c1ccc(-c2cccc(-c3oc(-c4cc(-c5ccccc5)ccc4-c4ccccc4)c4ccccc34)c2)cc1 ; c1ccc(-c2cccc(-c3oc(-c4ccc(-c5ccccc5)cc4-c4ccccc4)c4ccccc34)c2)cc1 ; c1ccc(-c2cccc(-c3oc(-c4cccc(-c5ccccc5)c4-c4ccccc4)c4ccccc34)c2)cc1]

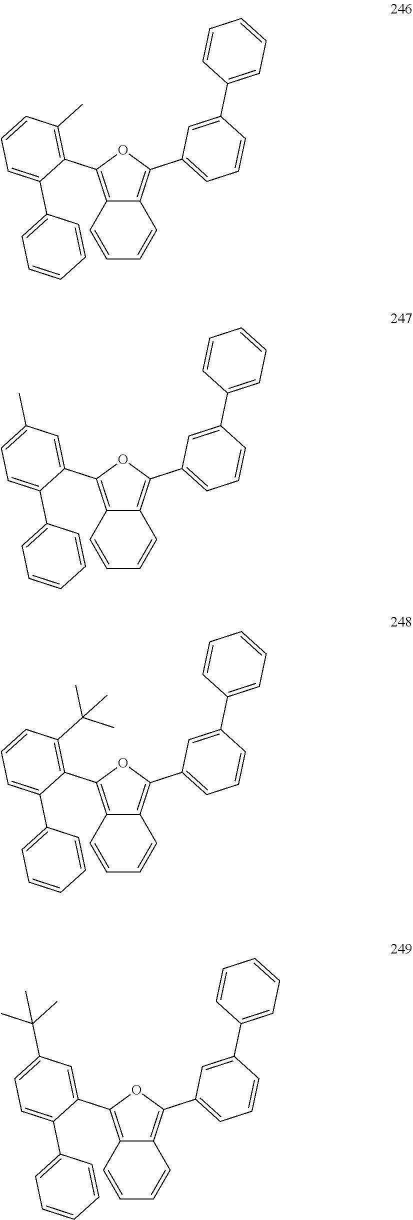 [SMILES: Cc1cccc(-c2ccccc2)c1-c1oc(-c2cccc(-c3ccccc3)c2)c2ccccc12 ; Cc1ccc(-c2ccccc2)c(-c2oc(-c3cccc(-c4ccccc4)c3)c3ccccc23)c1 ; CC(C)(C)c1cccc(-c2ccccc2)c1-c1oc(-c2cccc(-c3ccccc3)c2)c2ccccc12 ; CC(C)(C)c1ccc(-c2ccccc2)c(-c2oc(-c3cccc(-c4ccccc4)c3)c3ccccc23)c1]

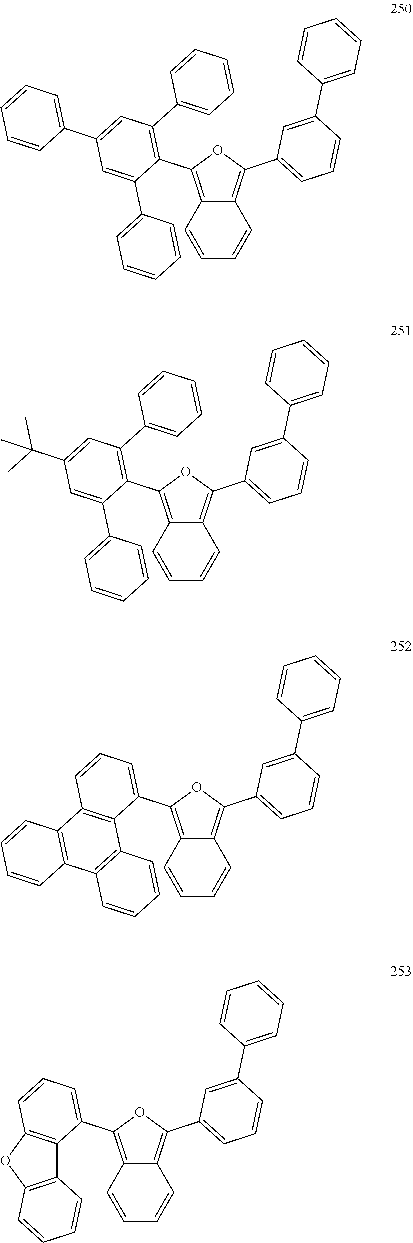 [SMILES: c1ccc(-c2cccc(-c3oc(-c4c(-c5ccccc5)cc(-c5ccccc5)cc4-c4ccccc4)c4ccccc34)c2)cc1 ; CC(C)(C)c1cc(-c2ccccc2)c(-c2oc(-c3cccc(-c4ccccc4)c3)c3ccccc23)c(-c2ccccc2)c1 ; c1ccc(-c2cccc(-c3oc(-c4cccc5c6ccccc6c6ccccc6c45)c4ccccc34)c2)cc1 ; c1ccc(-c2cccc(-c3oc(-c4cccc5oc6ccccc6c45)c4ccccc34)c2)cc1]

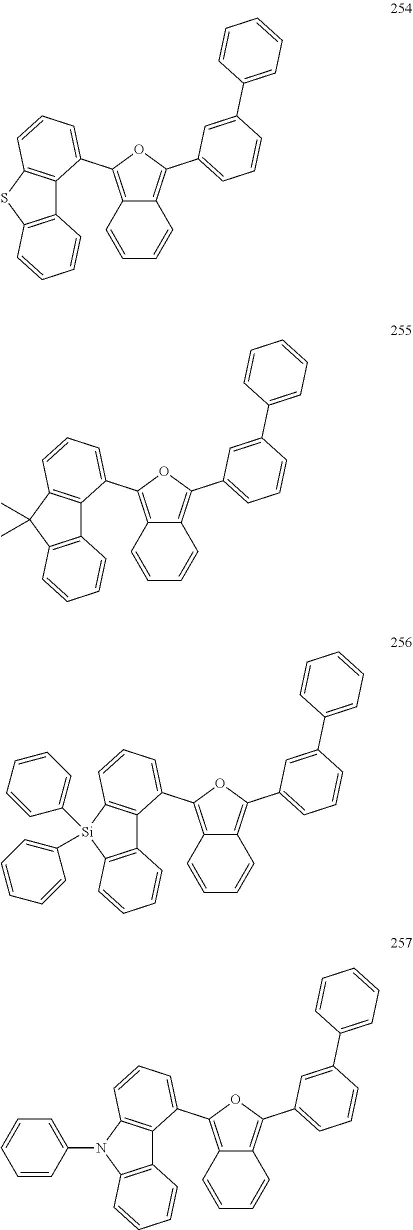 [SMILES: c1ccc(-c2cccc(-c3oc(-c4cccc5sc6ccccc6c45)c4ccccc34)c2)cc1 ; CC1(C)c2ccccc2-c2c(-c3oc(-c4cccc(-c5ccccc5)c4)c4ccccc34)cccc21 ; c1ccc(-c2cccc(-c3oc(-c4cccc5c4-c4ccccc4[Si]5(c4ccccc4)c4ccccc4)c4ccccc34)c2)cc1 ; c1ccc(-c2cccc(-c3oc(-c4cccc5c4c4ccccc4n5-c4ccccc4)c4ccccc34)c2)cc1]

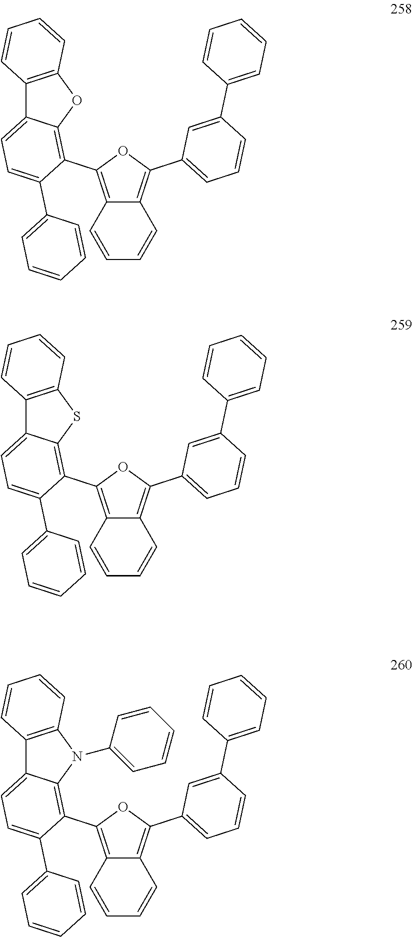 [SMILES: c1ccc(-c2cccc(-c3oc(-c4c(-c5ccccc5)ccc5c4oc4ccccc45)c4ccccc34)c2)cc1 ; c1ccc(-c2cccc(-c3oc(-c4c(-c5ccccc5)ccc5c4sc4ccccc45)c4ccccc34)c2)cc1 ; c1ccc(-c2cccc(-c3oc(-c4c(-c5ccccc5)ccc5c6ccccc6n(-c6ccccc6)c45)c4ccccc34)c2)cc1]

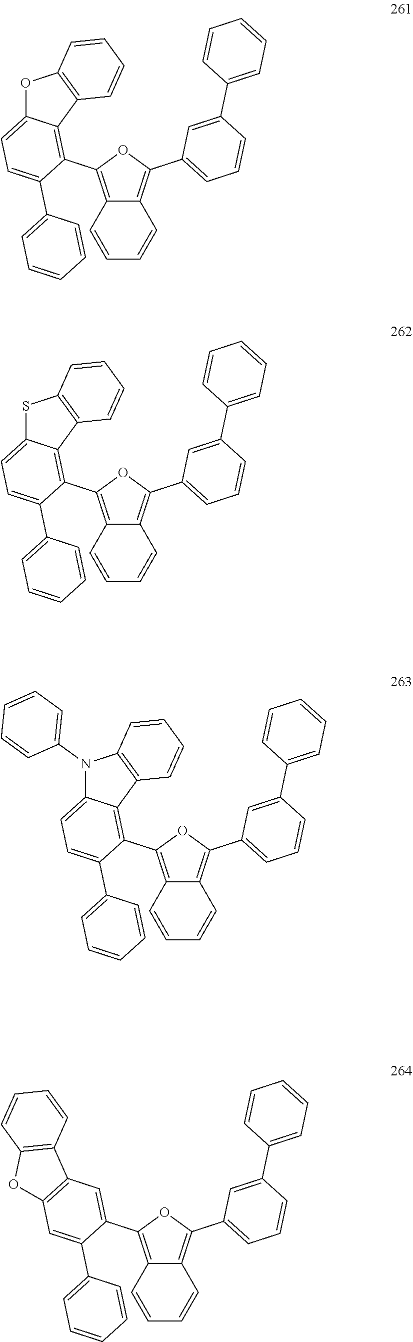 [SMILES: c1ccc(-c2cccc(-c3oc(-c4c(-c5ccccc5)ccc5oc6ccccc6c45)c4ccccc34)c2)cc1 ; c1ccc(-c2cccc(-c3oc(-c4c(-c5ccccc5)ccc5sc6ccccc6c45)c4ccccc34)c2)cc1 ; c1ccc(-c2cccc(-c3oc(-c4c(-c5ccccc5)ccc5c4c4ccccc4n5-c4ccccc4)c4ccccc34)c2)cc1 ; c1ccc(-c2cccc(-c3oc(-c4cc5c(cc4-c4ccccc4)oc4ccccc45)c4ccccc34)c2)cc1]

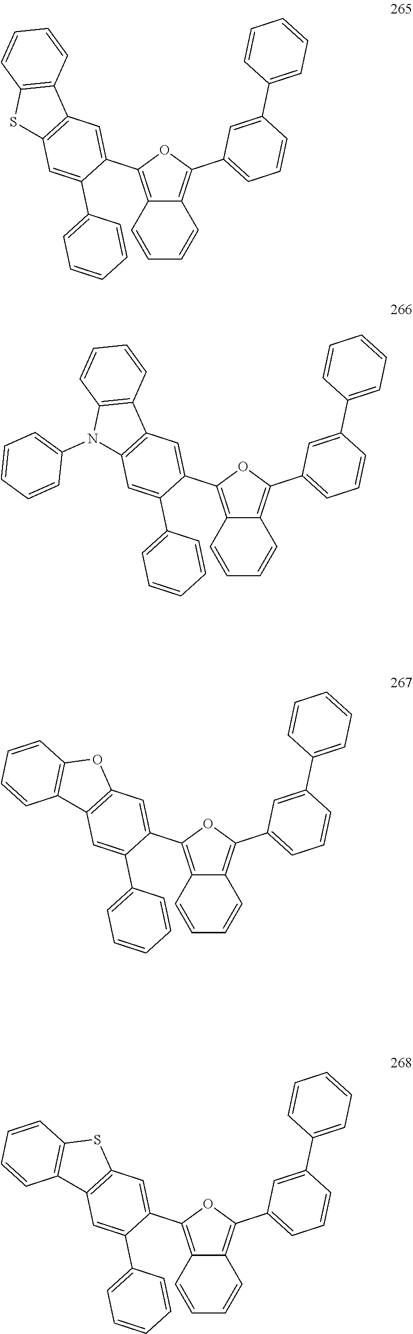 [SMILES: c1ccc(-c2cccc(-c3oc(-c4cc5c(cc4-c4ccccc4)sc4ccccc45)c4ccccc34)c2)cc1 ; c1ccc(-c2cccc(-c3oc(-c4cc5c6ccccc6n(-c6ccccc6)c5cc4-c4ccccc4)c4ccccc34)c2)cc1 ; c1ccc(-c2cccc(-c3oc(-c4cc5oc6ccccc6c5cc4-c4ccccc4)c4ccccc34)c2)cc1 ; c1ccc(-c2cccc(-c3oc(-c4cc5sc6ccccc6c5cc4-c4ccccc4)c4ccccc34)c2)cc1]

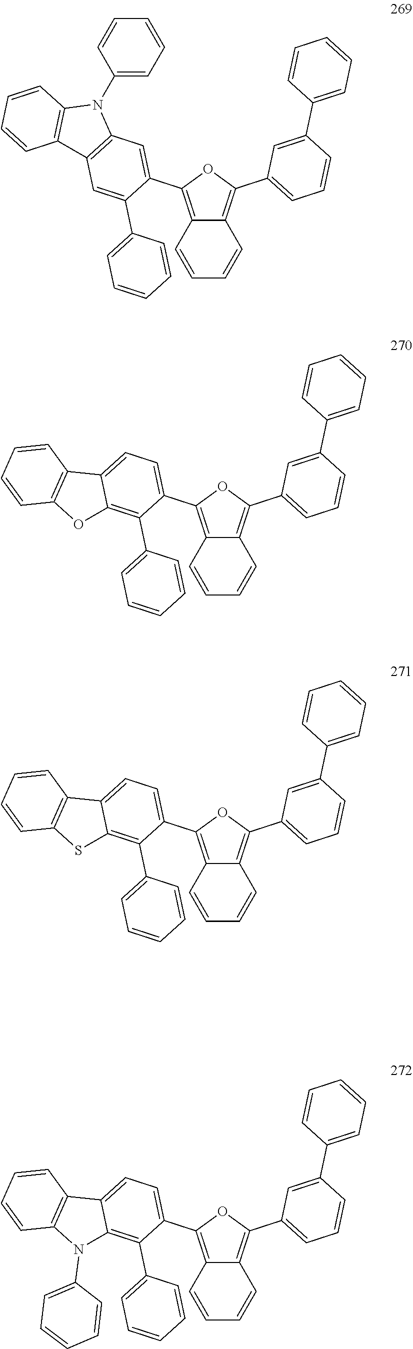 [SMILES: c1ccc(-c2cccc(-c3oc(-c4cc5c(cc4-c4ccccc4)c4ccccc4n5-c4ccccc4)c4ccccc34)c2)cc1 ; c1ccc(-c2cccc(-c3oc(-c4ccc5c(oc6ccccc65)c4-c4ccccc4)c4ccccc34)c2)cc1 ; c1ccc(-c2cccc(-c3oc(-c4ccc5c(sc6ccccc65)c4-c4ccccc4)c4ccccc34)c2)cc1 ; c1ccc(-c2cccc(-c3oc(-c4ccc5c6ccccc6n(-c6ccccc6)c5c4-c4ccccc4)c4ccccc34)c2)cc1]

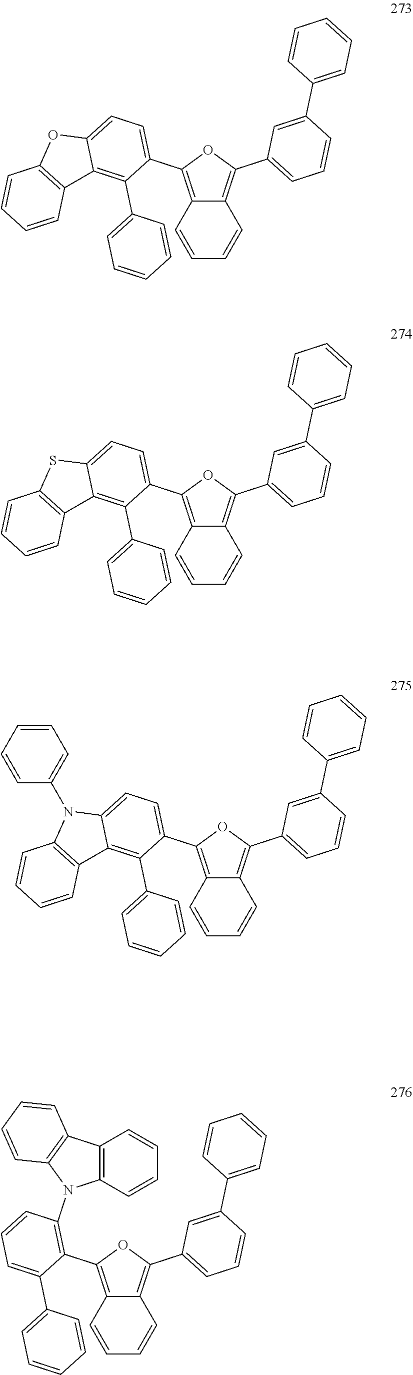 [SMILES: c1ccc(-c2cccc(-c3oc(-c4ccc5oc6ccccc6c5c4-c4ccccc4)c4ccccc34)c2)cc1 ; c1ccc(-c2cccc(-c3oc(-c4ccc5sc6ccccc6c5c4-c4ccccc4)c4ccccc34)c2)cc1 ; c1ccc(-c2cccc(-c3oc(-c4ccc5c(c4-c4ccccc4)c4ccccc4n5-c4ccccc4)c4ccccc34)c2)cc1 ; c1ccc(-c2cccc(-c3oc(-c4c(-c5ccccc5)cccc4-n4c5ccccc5c5ccccc54)c4ccccc34)c2)cc1]

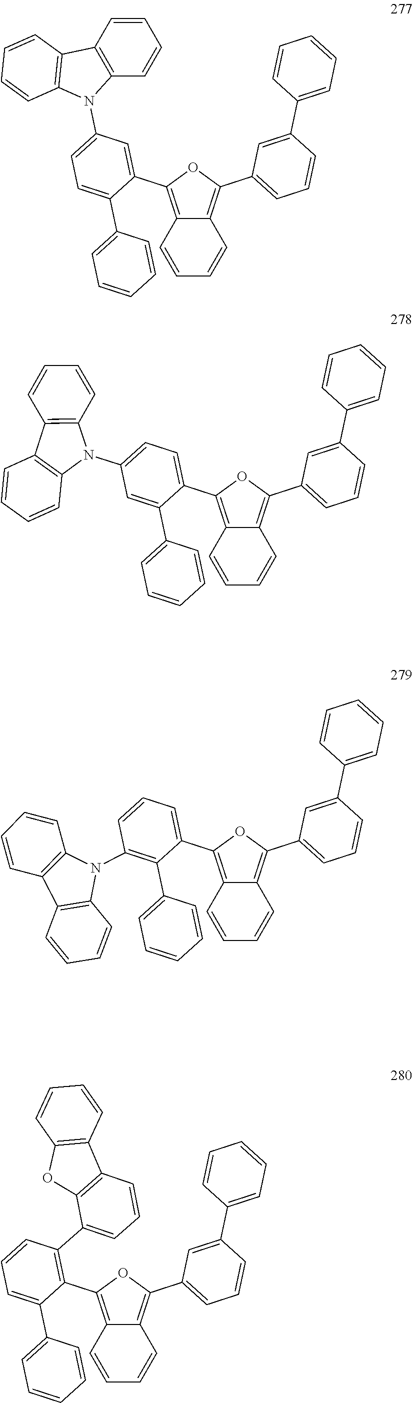 [SMILES: c1ccc(-c2cccc(-c3oc(-c4cc(-n5c6ccccc6c6ccccc65)ccc4-c4ccccc4)c4ccccc34)c2)cc1 ; c1ccc(-c2cccc(-c3oc(-c4ccc(-n5c6ccccc6c6ccccc65)cc4-c4ccccc4)c4ccccc34)c2)cc1 ; c1ccc(-c2cccc(-c3oc(-c4cccc(-n5c6ccccc6c6ccccc65)c4-c4ccccc4)c4ccccc34)c2)cc1 ; c1ccc(-c2cccc(-c3oc(-c4c(-c5ccccc5)cccc4-c4cccc5c4oc4ccccc45)c4ccccc34)c2)cc1]

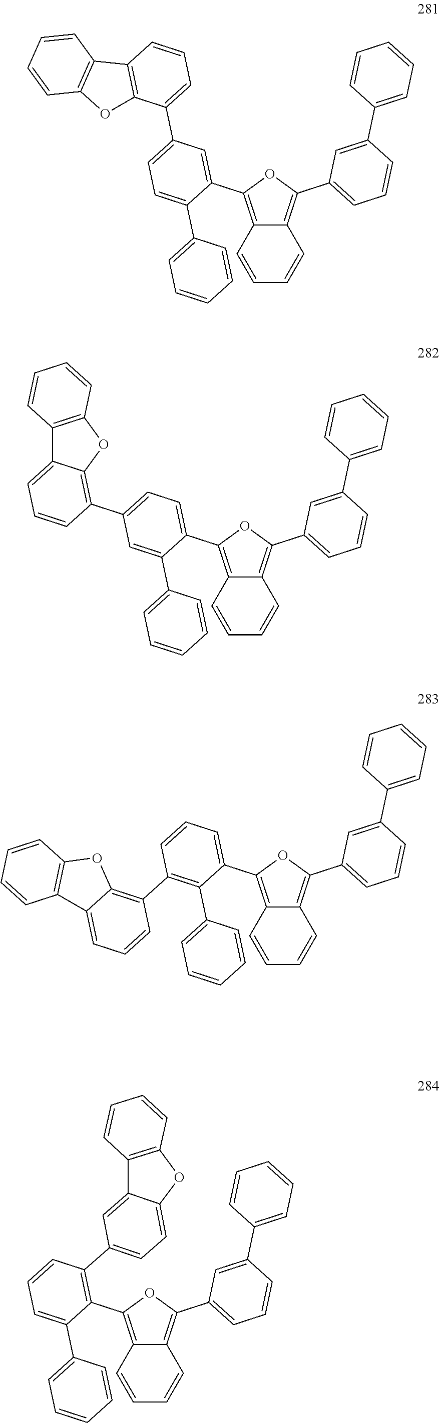 [SMILES: c1ccc(-c2cccc(-c3oc(-c4cc(-c5cccc6c5oc5ccccc56)ccc4-c4ccccc4)c4ccccc34)c2)cc1 ; c1ccc(-c2cccc(-c3oc(-c4ccc(-c5cccc6c5oc5ccccc56)cc4-c4ccccc4)c4ccccc34)c2)cc1 ; c1ccc(-c2cccc(-c3oc(-c4cccc(-c5cccc6c5oc5ccccc56)c4-c4ccccc4)c4ccccc34)c2)cc1 ; c1ccc(-c2cccc(-c3oc(-c4c(-c5ccccc5)cccc4-c4ccc5oc6ccccc6c5c4)c4ccccc34)c2)cc1]

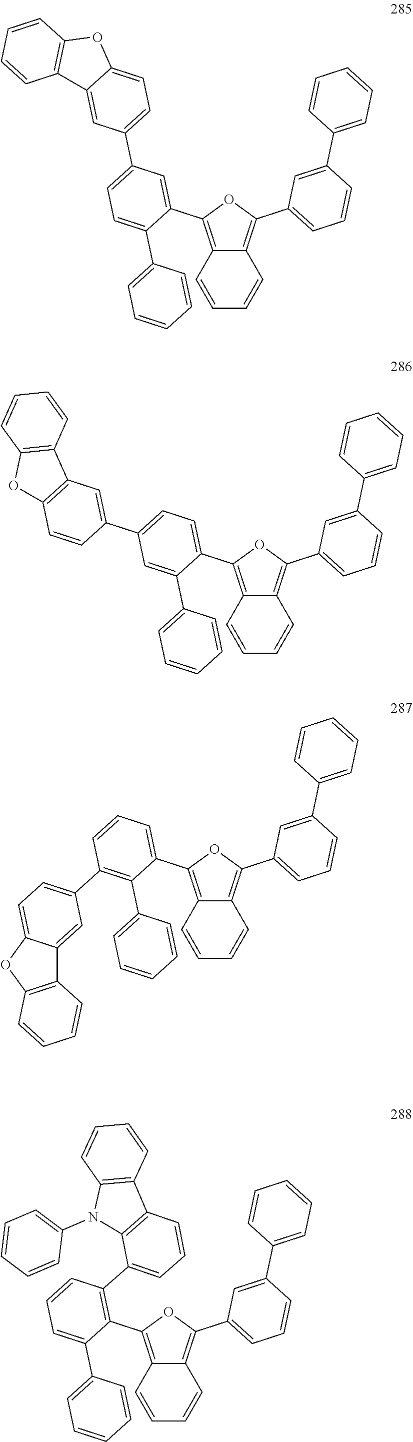 [SMILES: c1ccc(-c2cccc(-c3oc(-c4cc(-c5ccc6oc7ccccc7c6c5)ccc4-c4ccccc4)c4ccccc34)c2)cc1 ; c1ccc(-c2cccc(-c3oc(-c4ccc(-c5ccc6oc7ccccc7c6c5)cc4-c4ccccc4)c4ccccc34)c2)cc1 ; c1ccc(-c2cccc(-c3oc(-c4cccc(-c5ccc6oc7ccccc7c6c5)c4-c4ccccc4)c4ccccc34)c2)cc1 ; c1ccc(-c2cccc(-c3oc(-c4c(-c5ccccc5)cccc4-c4cccc5c6ccccc6n(-c6ccccc6)c45)c4ccccc34)c2)cc1]

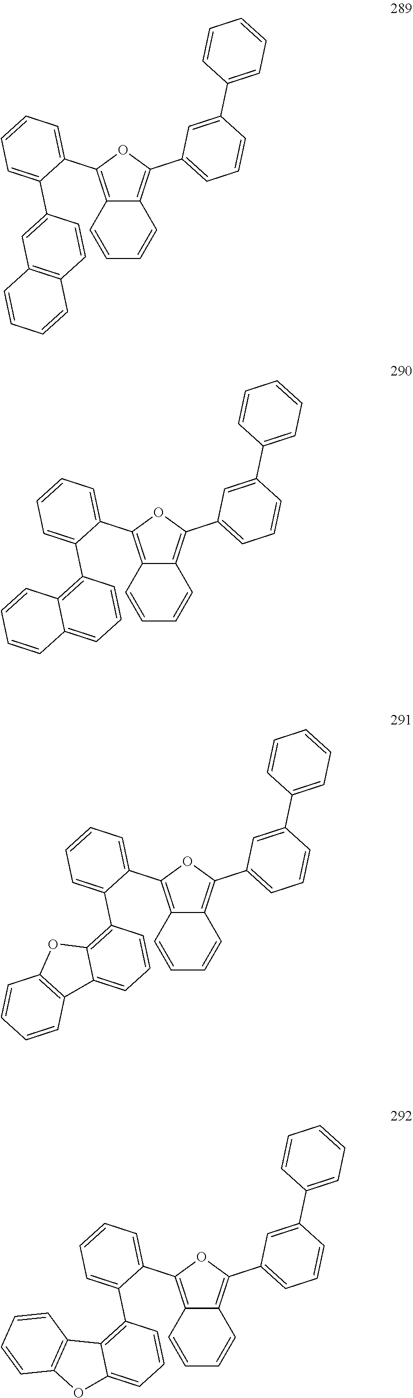 [SMILES: c1ccc(-c2cccc(-c3oc(-c4ccccc4-c4ccc5ccccc5c4)c4ccccc34)c2)cc1 ; c1ccc(-c2cccc(-c3oc(-c4ccccc4-c4cccc5ccccc45)c4ccccc34)c2)cc1 ; c1ccc(-c2cccc(-c3oc(-c4ccccc4-c4cccc5c4oc4ccccc45)c4ccccc34)c2)cc1 ; c1ccc(-c2cccc(-c3oc(-c4ccccc4-c4cccc5oc6ccccc6c45)c4ccccc34)c2)cc1]

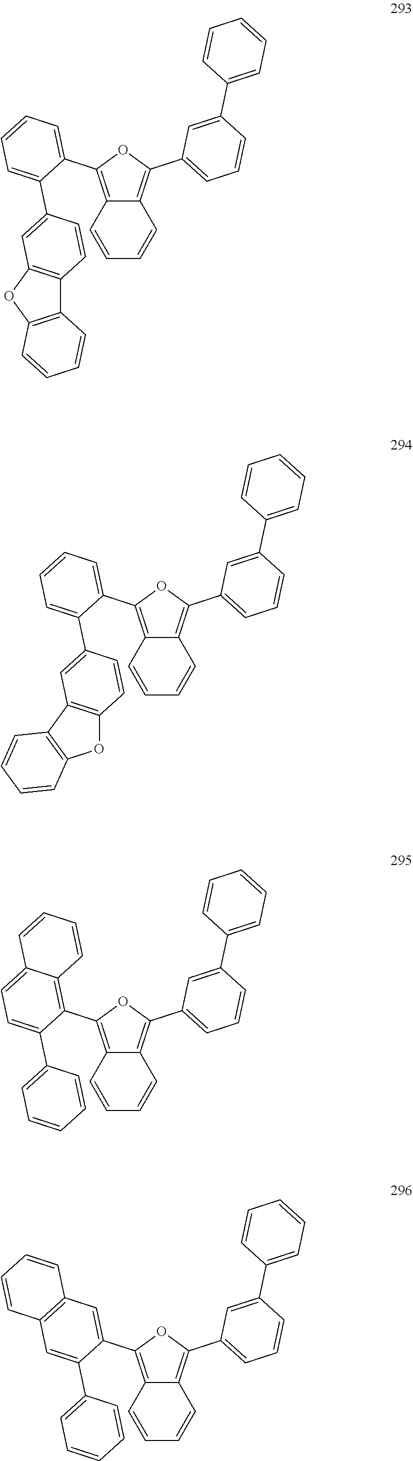 [SMILES: c1ccc(-c2cccc(-c3oc(-c4ccccc4-c4ccc5c(c4)oc4ccccc45)c4ccccc34)c2)cc1 ; c1ccc(-c2cccc(-c3oc(-c4ccccc4-c4ccc5oc6ccccc6c5c4)c4ccccc34)c2)cc1 ; c1ccc(-c2cccc(-c3oc(-c4c(-c5ccccc5)ccc5ccccc45)c4ccccc34)c2)cc1 ; c1ccc(-c2cccc(-c3oc(-c4cc5ccccc5cc4-c4ccccc4)c4ccccc34)c2)cc1]

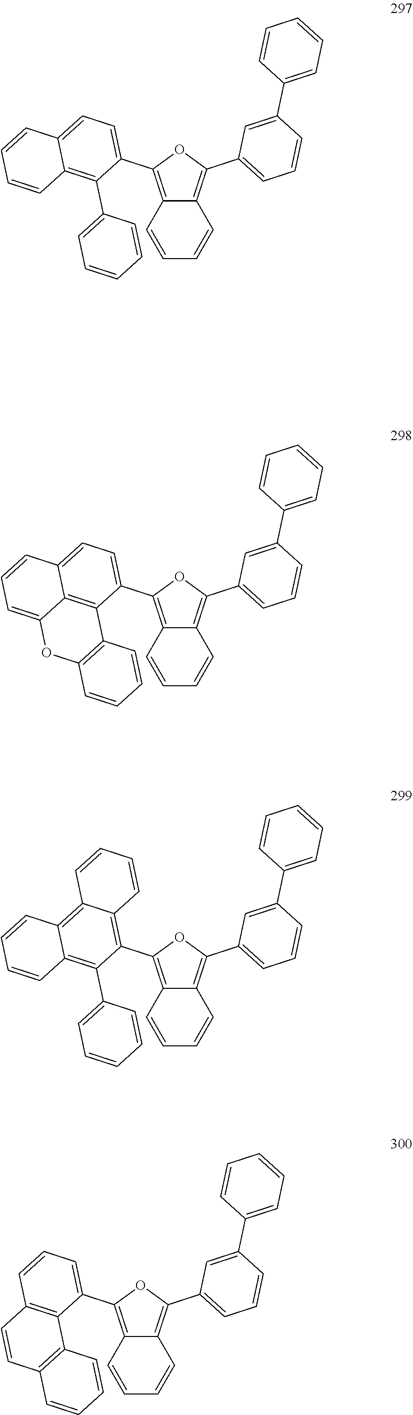 [SMILES: c1ccc(-c2cccc(-c3oc(-c4ccc5ccccc5c4-c4ccccc4)c4ccccc34)c2)cc1 ; c1ccc(-c2cccc(-c3oc(-c4ccc5cccc6c5c4-c4ccccc4O6)c4ccccc34)c2)cc1 ; c1ccc(-c2cccc(-c3oc(-c4c(-c5ccccc5)c5ccccc5c5ccccc45)c4ccccc34)c2)cc1 ; c1ccc(-c2cccc(-c3oc(-c4cccc5ccc6ccccc6c45)c4ccccc34)c2)cc1]

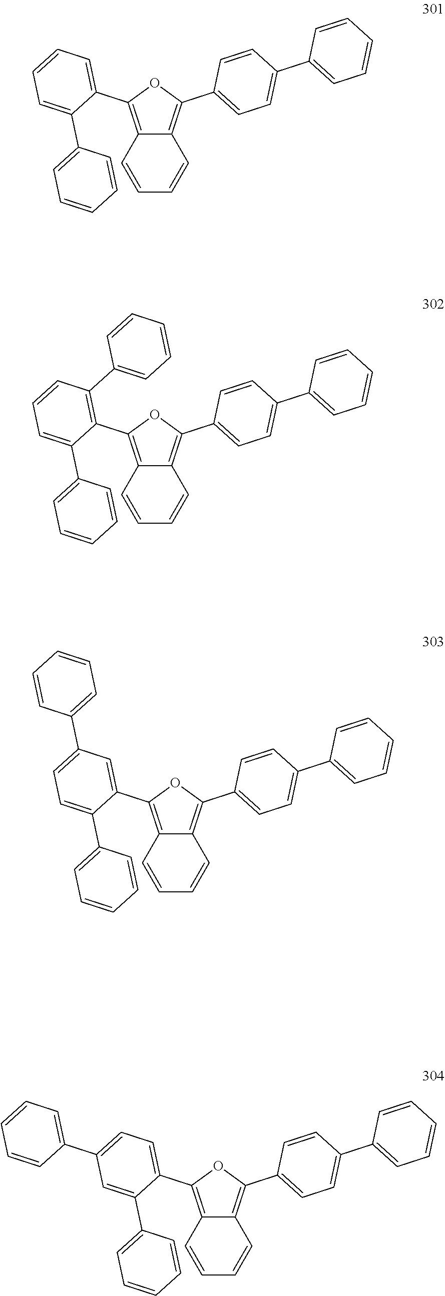 [SMILES: c1ccc(-c2ccc(-c3oc(-c4ccccc4-c4ccccc4)c4ccccc34)cc2)cc1 ; c1ccc(-c2ccc(-c3oc(-c4c(-c5ccccc5)cccc4-c4ccccc4)c4ccccc34)cc2)cc1 ; c1ccc(-c2ccc(-c3oc(-c4cc(-c5ccccc5)ccc4-c4ccccc4)c4ccccc34)cc2)cc1 ; c1ccc(-c2ccc(-c3oc(-c4ccc(-c5ccccc5)cc4-c4ccccc4)c4ccccc34)cc2)cc1]

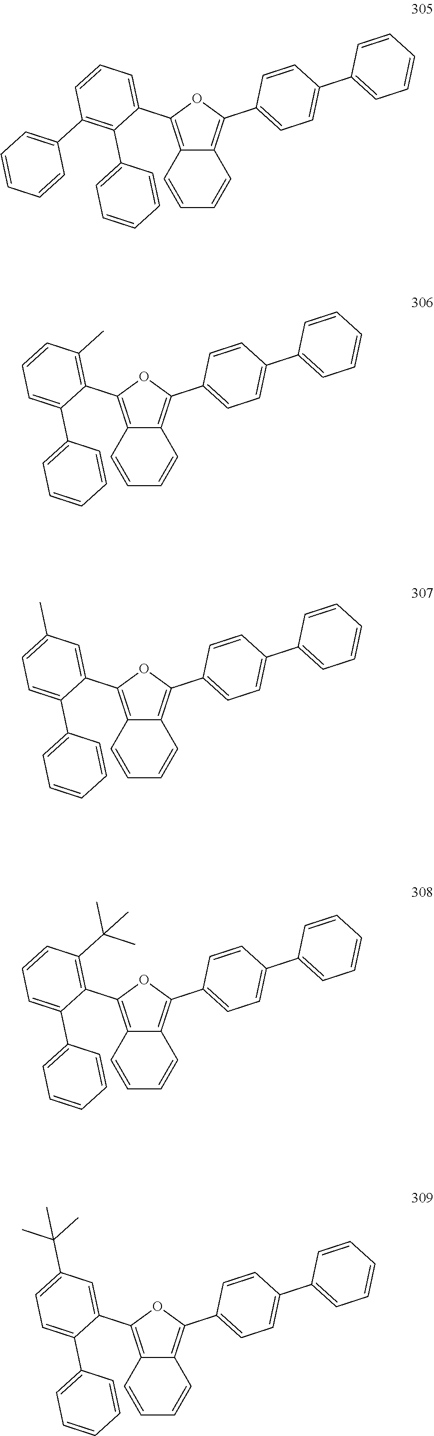 [SMILES: c1ccc(-c2ccc(-c3oc(-c4cccc(-c5ccccc5)c4-c4ccccc4)c4ccccc34)cc2)cc1 ; Cc1cccc(-c2ccccc2)c1-c1oc(-c2ccc(-c3ccccc3)cc2)c2ccccc12 ; Cc1ccc(-c2ccccc2)c(-c2oc(-c3ccc(-c4ccccc4)cc3)c3ccccc23)c1 ; CC(C)(C)c1cccc(-c2ccccc2)c1-c1oc(-c2ccc(-c3ccccc3)cc2)c2ccccc12 ; CC(C)(C)c1ccc(-c2ccccc2)c(-c2oc(-c3ccc(-c4ccccc4)cc3)c3ccccc23)c1]

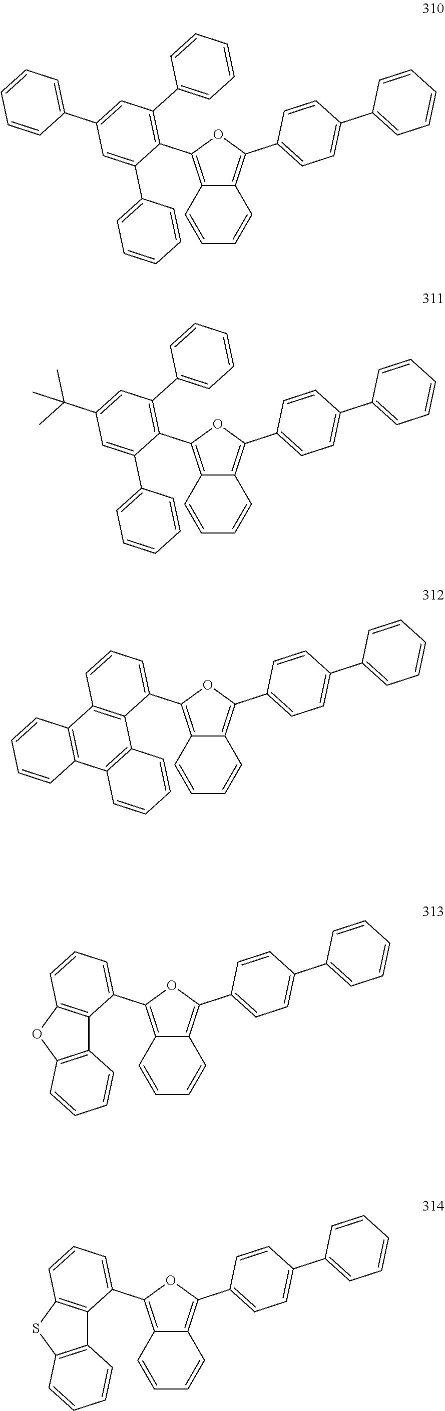 [SMILES: c1ccc(-c2ccc(-c3oc(-c4c(-c5ccccc5)cc(-c5ccccc5)cc4-c4ccccc4)c4ccccc34)cc2)cc1 ; CC(C)(C)c1cc(-c2ccccc2)c(-c2oc(-c3ccc(-c4ccccc4)cc3)c3ccccc23)c(-c2ccccc2)c1 ; c1ccc(-c2ccc(-c3oc(-c4cccc5c6ccccc6c6ccccc6c45)c4ccccc34)cc2)cc1 ; c1ccc(-c2ccc(-c3oc(-c4cccc5oc6ccccc6c45)c4ccccc34)cc2)cc1 ; c1ccc(-c2ccc(-c3oc(-c4cccc5sc6ccccc6c45)c4ccccc34)cc2)cc1]

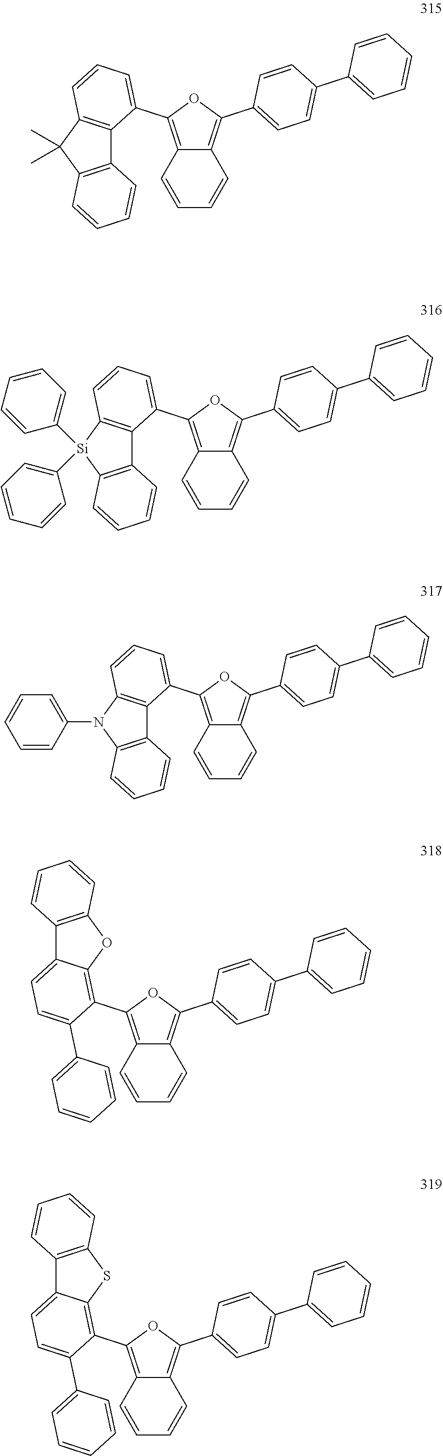 [SMILES: CC1(C)c2ccccc2-c2c(-c3oc(-c4ccc(-c5ccccc5)cc4)c4ccccc34)cccc21 ; c1ccc(-c2ccc(-c3oc(-c4cccc5c4-c4ccccc4[Si]5(c4ccccc4)c4ccccc4)c4ccccc34)cc2)cc1 ; c1ccc(-c2ccc(-c3oc(-c4cccc5c4c4ccccc4n5-c4ccccc4)c4ccccc34)cc2)cc1 ; c1ccc(-c2ccc(-c3oc(-c4c(-c5ccccc5)ccc5c4oc4ccccc45)c4ccccc34)cc2)cc1 ; c1ccc(-c2ccc(-c3oc(-c4c(-c5ccccc5)ccc5c4sc4ccccc45)c4ccccc34)cc2)cc1]

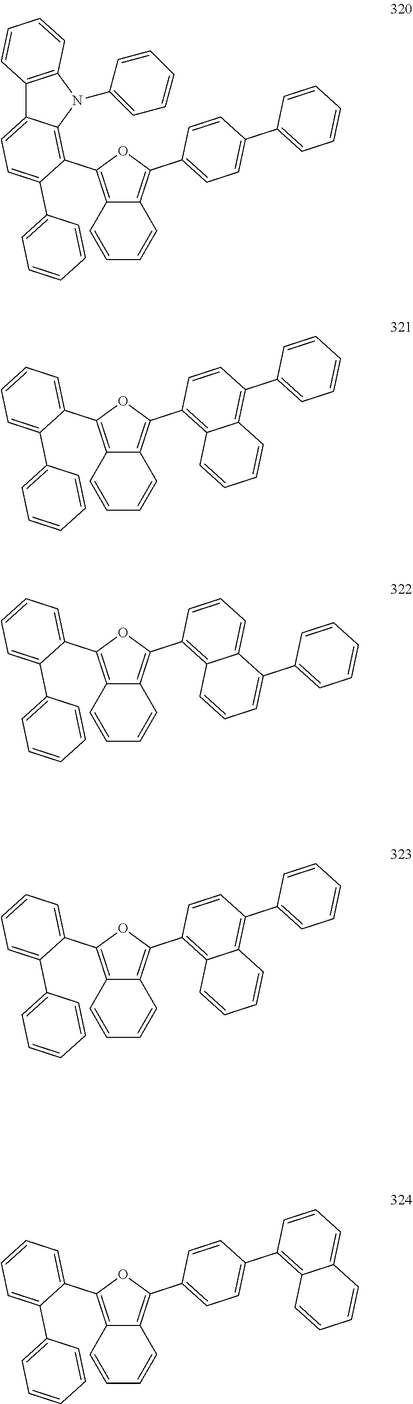 [SMILES: c1ccc(-c2ccc(-c3oc(-c4c(-c5ccccc5)ccc5c6ccccc6n(-c6ccccc6)c45)c4ccccc34)cc2)cc1 ; c1ccc(-c2ccccc2-c2oc(-c3ccc(-c4ccccc4)c4ccccc34)c3ccccc23)cc1 ; c1ccc(-c2ccccc2-c2oc(-c3cccc4c(-c5ccccc5)cccc34)c3ccccc23)cc1 ; c1ccc(-c2ccccc2-c2oc(-c3ccc(-c4ccccc4)c4ccccc34)c3ccccc23)cc1 ; c1ccc(-c2ccccc2-c2oc(-c3ccc(-c4cccc5ccccc45)cc3)c3ccccc23)cc1]

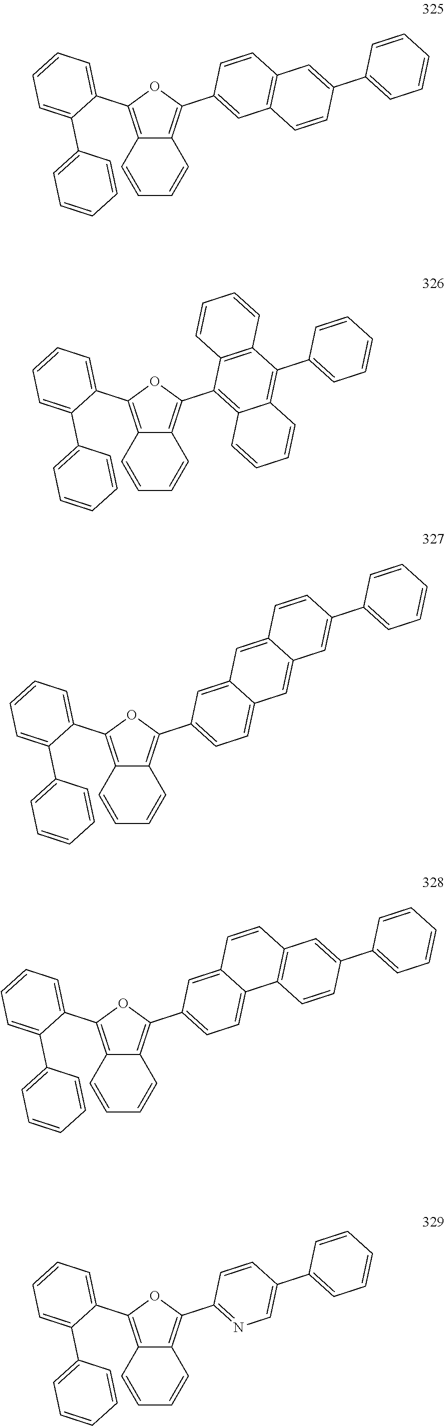 [SMILES: c1ccc(-c2ccc3cc(-c4oc(-c5ccccc5-c5ccccc5)c5ccccc45)ccc3c2)cc1 ; c1ccc(-c2ccccc2-c2oc(-c3c4ccccc4c(-c4ccccc4)c4ccccc34)c3ccccc23)cc1 ; c1ccc(-c2ccc3cc4cc(-c5oc(-c6ccccc6-c6ccccc6)c6ccccc56)ccc4cc3c2)cc1 ; c1ccc(-c2ccc3c(ccc4cc(-c5oc(-c6ccccc6-c6ccccc6)c6ccccc56)ccc43)c2)cc1 ; c1ccc(-c2ccc(-c3oc(-c4ccccc4-c4ccccc4)c4ccccc34)nc2)cc1]

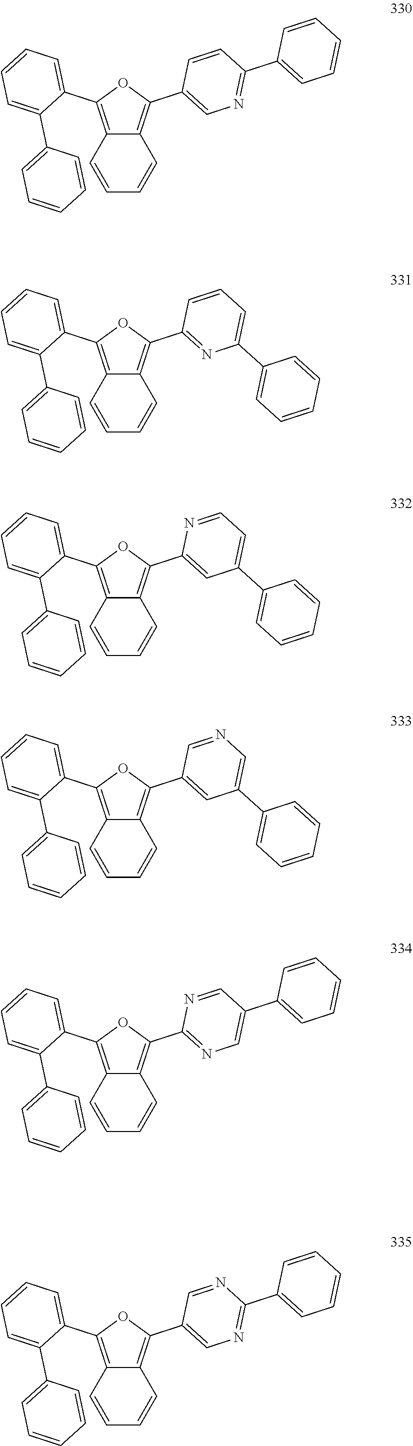 [SMILES: c1ccc(-c2ccc(-c3oc(-c4ccccc4-c4ccccc4)c4ccccc34)cn2)cc1 ; c1ccc(-c2cccc(-c3oc(-c4ccccc4-c4ccccc4)c4ccccc34)n2)cc1 ; c1ccc(-c2ccnc(-c3oc(-c4ccccc4-c4ccccc4)c4ccccc34)c2)cc1 ; c1ccc(-c2cncc(-c3oc(-c4ccccc4-c4ccccc4)c4ccccc34)c2)cc1 ; c1ccc(-c2cnc(-c3oc(-c4ccccc4-c4ccccc4)c4ccccc34)nc2)cc1 ; c1ccc(-c2ncc(-c3oc(-c4ccccc4-c4ccccc4)c4ccccc34)cn2)cc1]

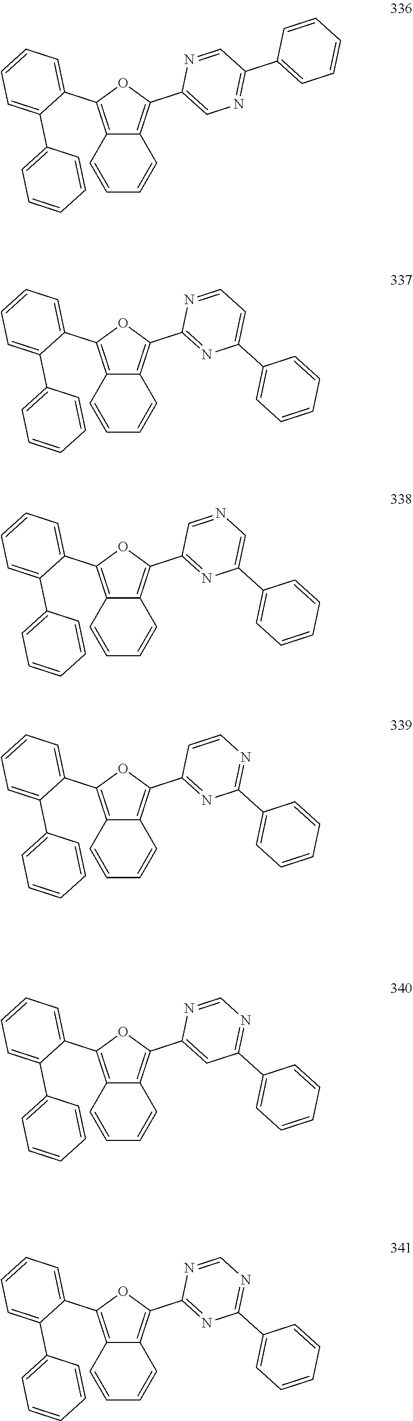 [SMILES: c1ccc(-c2cnc(-c3oc(-c4ccccc4-c4ccccc4)c4ccccc34)cn2)cc1 ; c1ccc(-c2ccnc(-c3oc(-c4ccccc4-c4ccccc4)c4ccccc34)n2)cc1 ; c1ccc(-c2cncc(-c3oc(-c4ccccc4-c4ccccc4)c4ccccc34)n2)cc1 ; c1ccc(-c2nccc(-c3oc(-c4ccccc4-c4ccccc4)c4ccccc34)n2)cc1 ; c1ccc(-c2cc(-c3oc(-c4ccccc4-c4ccccc4)c4ccccc34)ncn2)cc1 ; c1ccc(-c2ncnc(-c3oc(-c4ccccc4-c4ccccc4)c4ccccc34)n2)cc1]

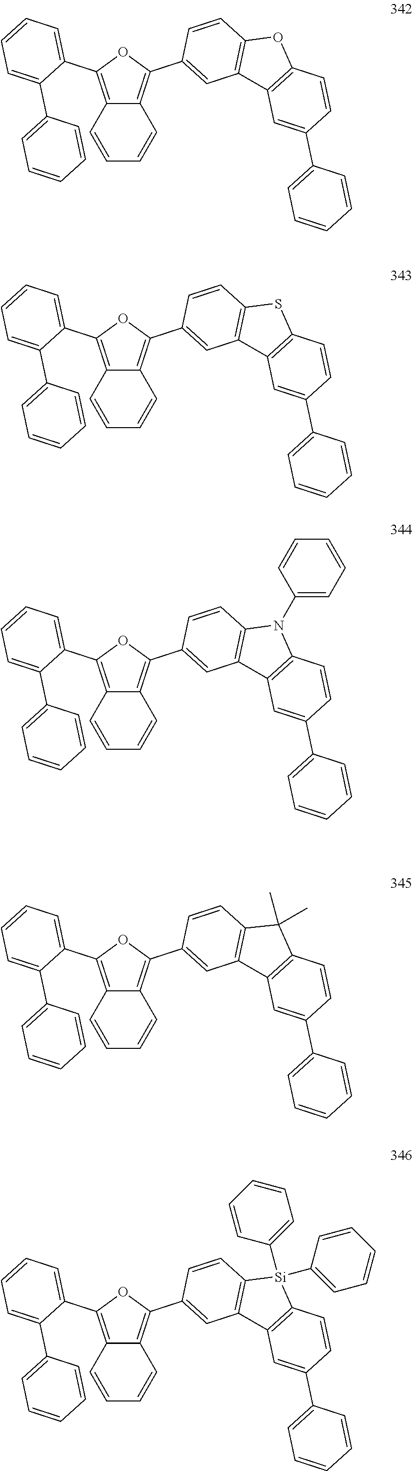

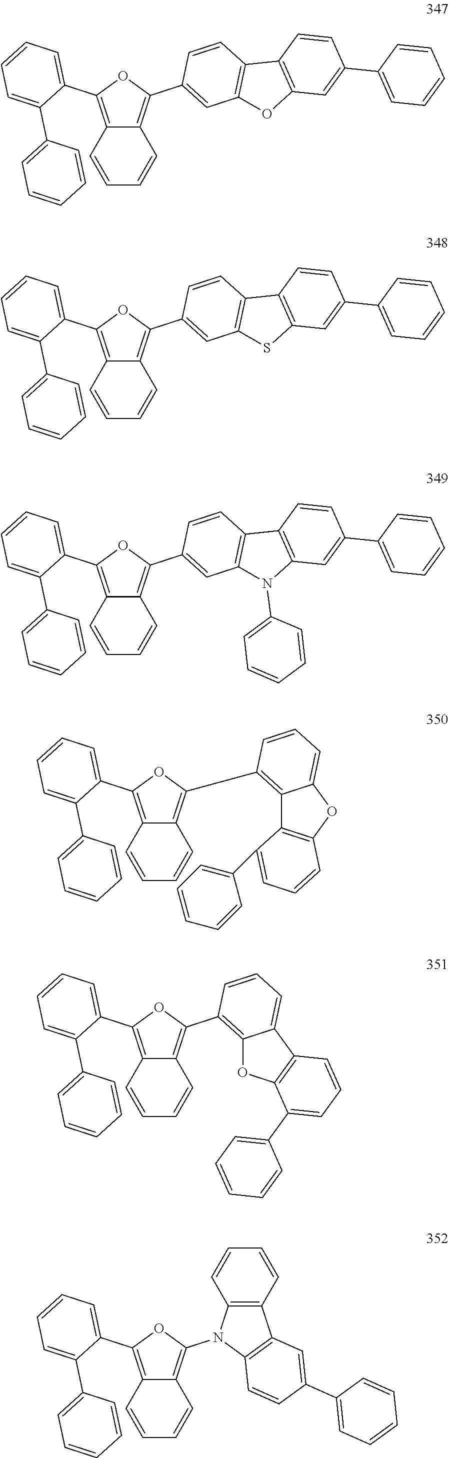 [SMILES: c1ccc(-c2ccc3c(c2)oc2cc(-c4oc(-c5ccccc5-c5ccccc5)c5ccccc45)ccc23)cc1 ; c1ccc(-c2ccc3c(c2)sc2cc(-c4oc(-c5ccccc5-c5ccccc5)c5ccccc45)ccc23)cc1 ; c1ccc(-c2ccc3c4ccc(-c5oc(-c6ccccc6-c6ccccc6)c6ccccc56)cc4n(-c4ccccc4)c3c2)cc1 ; c1ccc(-c2ccccc2-c2oc(-c3cccc4oc5cccc(-c6ccccc6)c5c34)c3ccccc23)cc1 ; c1ccc(-c2ccccc2-c2oc(-c3cccc4c3oc3c(-c5ccccc5)cccc34)c3ccccc23)cc1 ; c1ccc(-c2ccc3c(c2)c2ccccc2n3-c2oc(-c3ccccc3-c3ccccc3)c3ccccc23)cc1]

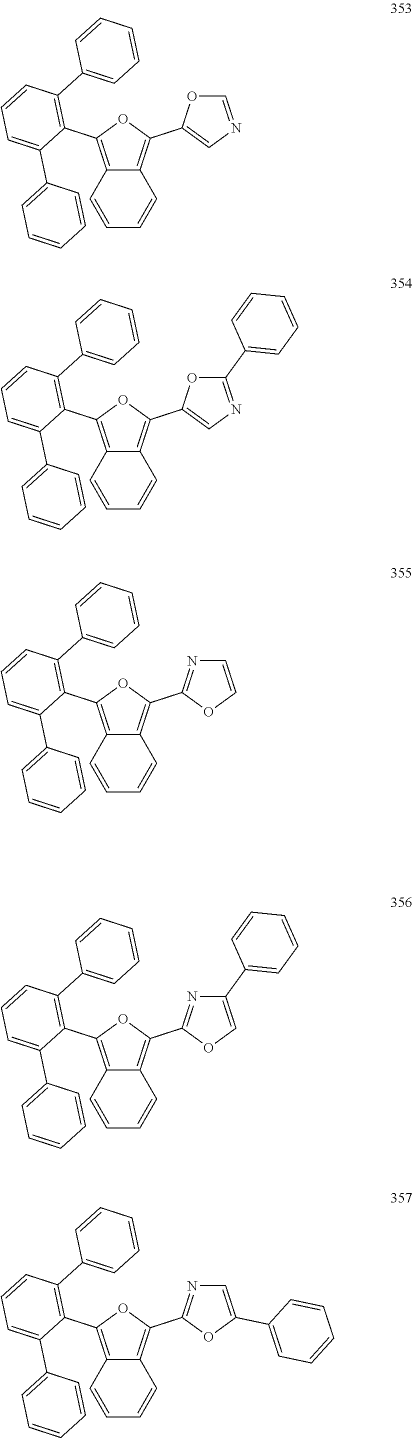 [SMILES: c1ccc(-c2cccc(-c3ccccc3)c2-c2oc(-c3cnco3)c3ccccc23)cc1 ; c1ccc(-c2ncc(-c3oc(-c4c(-c5ccccc5)cccc4-c4ccccc4)c4ccccc34)o2)cc1 ; c1ccc(-c2cccc(-c3ccccc3)c2-c2oc(-c3ncco3)c3ccccc23)cc1 ; c1ccc(-c2coc(-c3oc(-c4c(-c5ccccc5)cccc4-c4ccccc4)c4ccccc34)n2)cc1 ; c1ccc(-c2cnc(-c3oc(-c4c(-c5ccccc5)cccc4-c4ccccc4)c4ccccc34)o2)cc1]

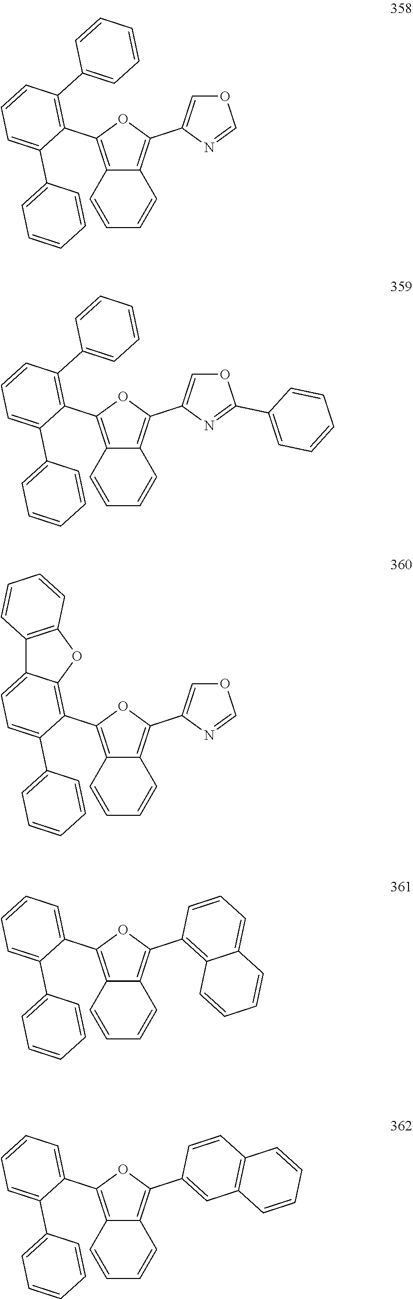 [SMILES: c1ccc(-c2cccc(-c3ccccc3)c2-c2oc(-c3cocn3)c3ccccc23)cc1 ; c1ccc(-c2nc(-c3oc(-c4c(-c5ccccc5)cccc4-c4ccccc4)c4ccccc34)co2)cc1 ; c1ccc(-c2ccc3c(oc4ccccc43)c2-c2oc(-c3cocn3)c3ccccc23)cc1 ; c1ccc(-c2ccccc2-c2oc(-c3cccc4ccccc34)c3ccccc23)cc1 ; c1ccc(-c2ccccc2-c2oc(-c3ccc4ccccc4c3)c3ccccc23)cc1]

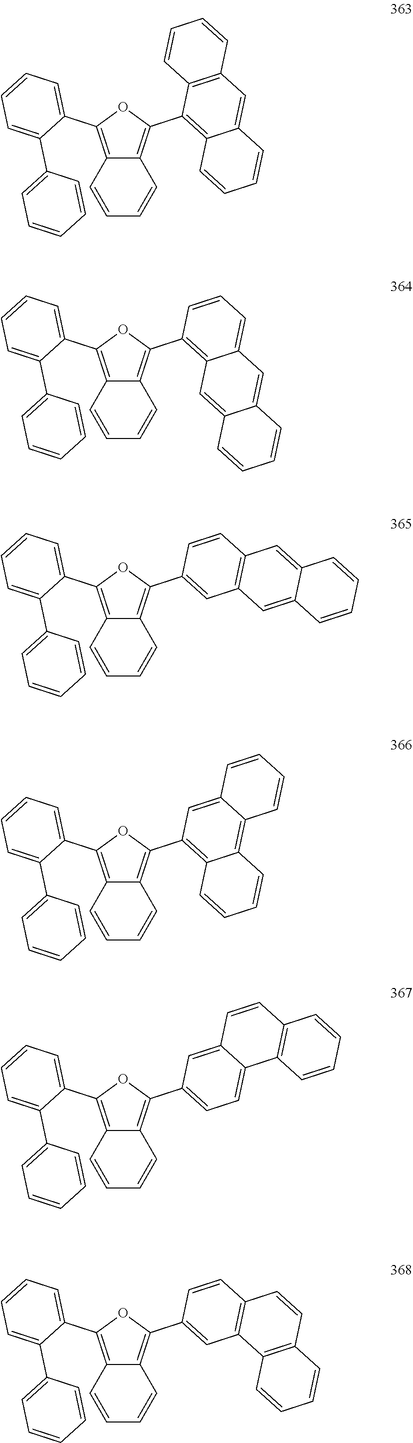 [SMILES: c1ccc(-c2ccccc2-c2oc(-c3c4ccccc4cc4ccccc34)c3ccccc23)cc1 ; c1ccc(-c2ccccc2-c2oc(-c3cccc4cc5ccccc5cc34)c3ccccc23)cc1 ; c1ccc(-c2ccccc2-c2oc(-c3ccc4cc5ccccc5cc4c3)c3ccccc23)cc1 ; c1ccc(-c2ccccc2-c2oc(-c3cc4ccccc4c4ccccc34)c3ccccc23)cc1 ; c1ccc(-c2ccccc2-c2oc(-c3ccc4c(ccc5ccccc54)c3)c3ccccc23)cc1 ; c1ccc(-c2ccccc2-c2oc(-c3ccc4ccc5ccccc5c4c3)c3ccccc23)cc1]

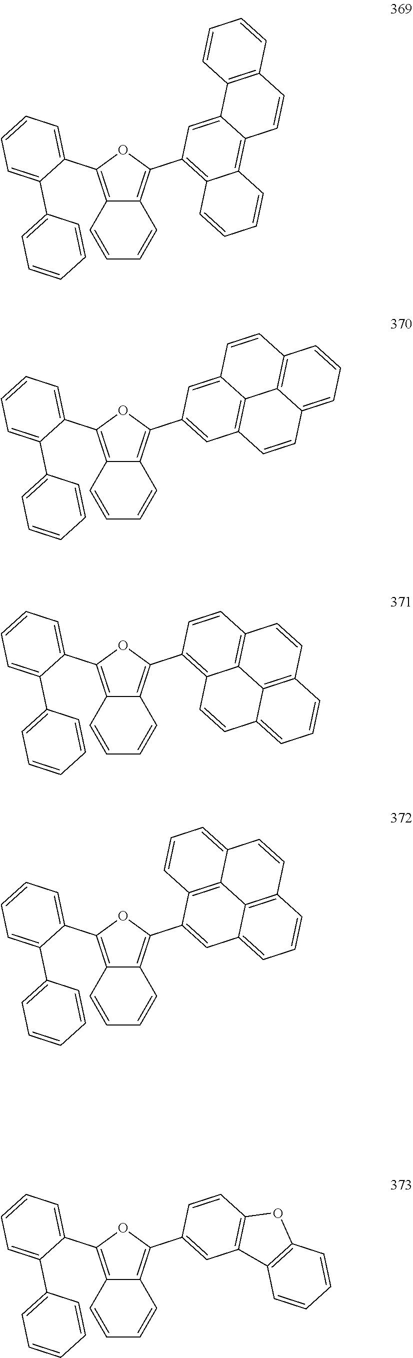 [SMILES: c1ccc(-c2ccccc2-c2oc(-c3cc4c5ccccc5ccc4c4ccccc34)c3ccccc23)cc1 ; c1ccc(-c2ccccc2-c2oc(-c3cc4ccc5cccc6ccc(c3)c4c56)c3ccccc23)cc1 ; c1ccc(-c2ccccc2-c2oc(-c3ccc4ccc5cccc6ccc3c4c56)c3ccccc23)cc1 ; c1ccc(-c2ccccc2-c2oc(-c3cc4cccc5ccc6cccc3c6c54)c3ccccc23)cc1 ; c1ccc(-c2ccccc2-c2oc(-c3ccc4oc5ccccc5c4c3)c3ccccc23)cc1]

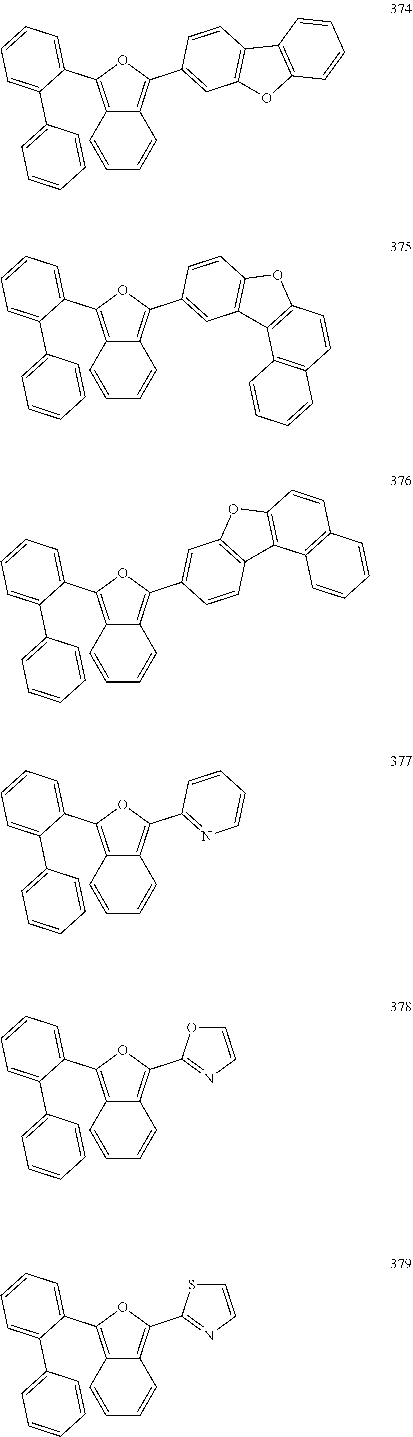 [SMILES: c1ccc(-c2ccccc2-c2oc(-c3ccc4c(c3)oc3ccccc34)c3ccccc23)cc1 ; c1ccc(-c2ccccc2-c2oc(-c3ccc4oc5ccc6ccccc6c5c4c3)c3ccccc23)cc1 ; c1ccc(-c2ccccc2-c2oc(-c3ccc4c(c3)oc3ccc5ccccc5c34)c3ccccc23)cc1 ; c1ccc(-c2ccccc2-c2oc(-c3ccccn3)c3ccccc23)cc1 ; c1ccc(-c2ccccc2-c2oc(-c3ncco3)c3ccccc23)cc1 ; c1ccc(-c2ccccc2-c2oc(-c3nccs3)c3ccccc23)cc1]

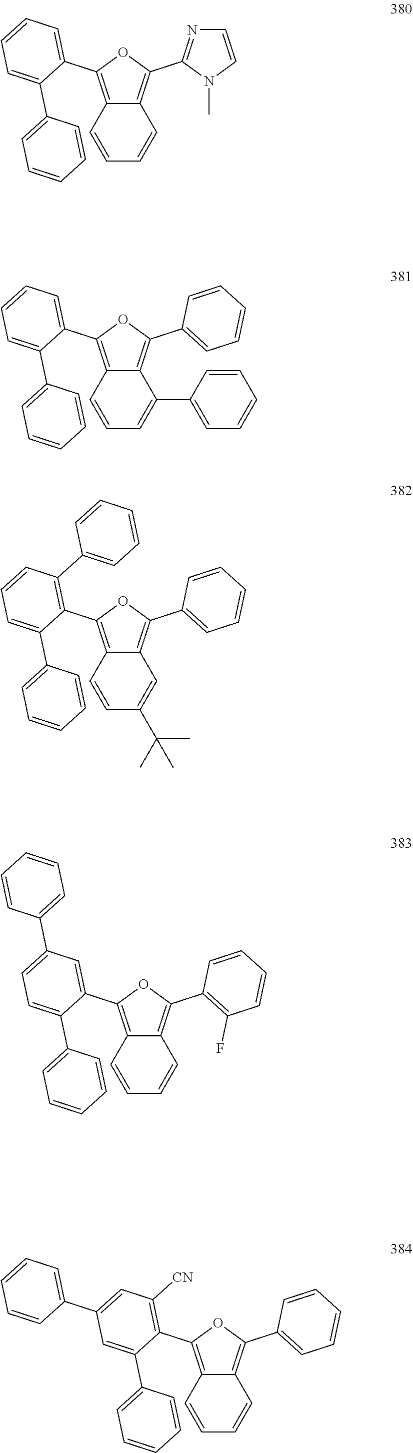 [SMILES: Cn1ccnc1-c1oc(-c2ccccc2-c2ccccc2)c2ccccc12 ; c1ccc(-c2ccccc2-c2oc(-c3ccccc3)c3c(-c4ccccc4)cccc23)cc1 ; CC(C)(C)c1ccc2c(-c3c(-c4ccccc4)cccc3-c3ccccc3)oc(-c3ccccc3)c2c1 ; Fc1ccccc1-c1oc(-c2cc(-c3ccccc3)ccc2-c2ccccc2)c2ccccc12 ; N#Cc1cc(-c2ccccc2)cc(-c2ccccc2)c1-c1oc(-c2ccccc2)c2ccccc12]

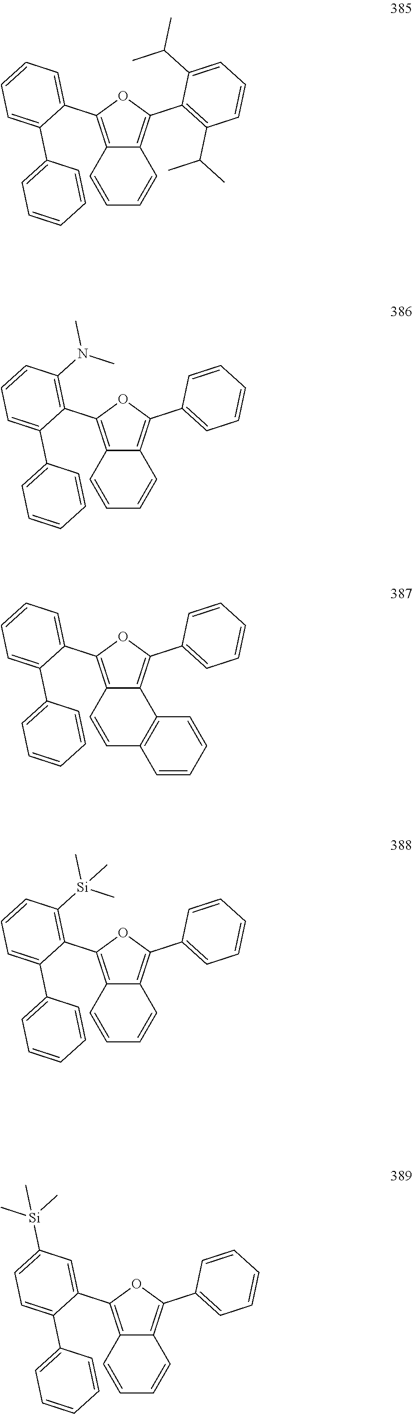 [SMILES: CC(C)c1cccc(C(C)C)c1-c1oc(-c2ccccc2-c2ccccc2)c2ccccc12 ; CN(C)c1cccc(-c2ccccc2)c1-c1oc(-c2ccccc2)c2ccccc12 ; c1ccc(-c2ccccc2-c2oc(-c3ccccc3)c3c2ccc2ccccc23)cc1 ; C[Si](C)(C)c1cccc(-c2ccccc2)c1-c1oc(-c2ccccc2)c2ccccc12 ; C[Si](C)(C)c1ccc(-c2ccccc2)c(-c2oc(-c3ccccc3)c3ccccc23)c1]

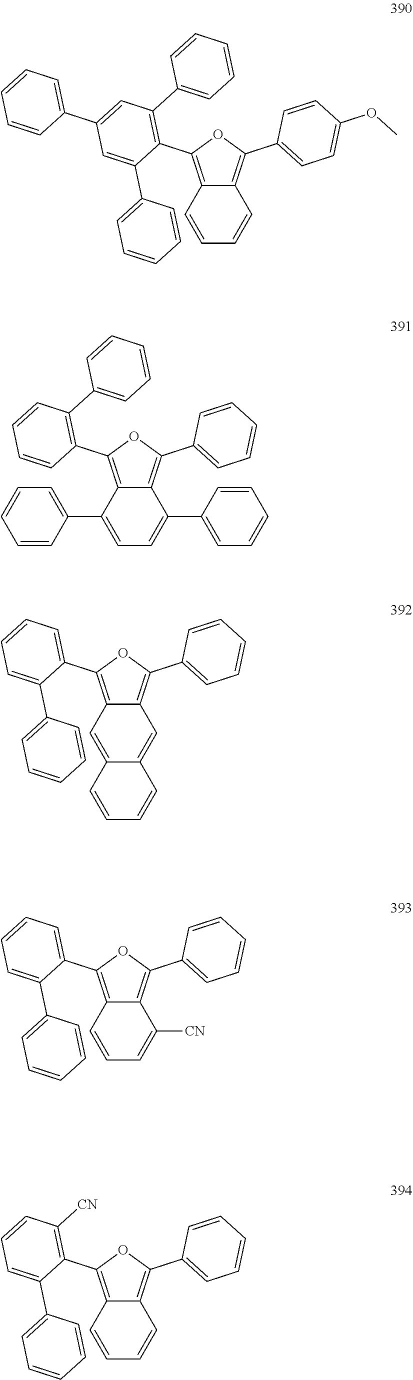 [SMILES: COc1ccc(-c2oc(-c3c(-c4ccccc4)cc(-c4ccccc4)cc3-c3ccccc3)c3ccccc23)cc1 ; c1ccc(-c2ccccc2-c2oc(-c3ccccc3)c3c(-c4ccccc4)ccc(-c4ccccc4)c23)cc1 ; c1ccc(-c2ccccc2-c2oc(-c3ccccc3)c3cc4ccccc4cc23)cc1 ; N#Cc1cccc2c(-c3ccccc3-c3ccccc3)oc(-c3ccccc3)c12 ; N#Cc1cccc(-c2ccccc2)c1-c1oc(-c2ccccc2)c2ccccc12]

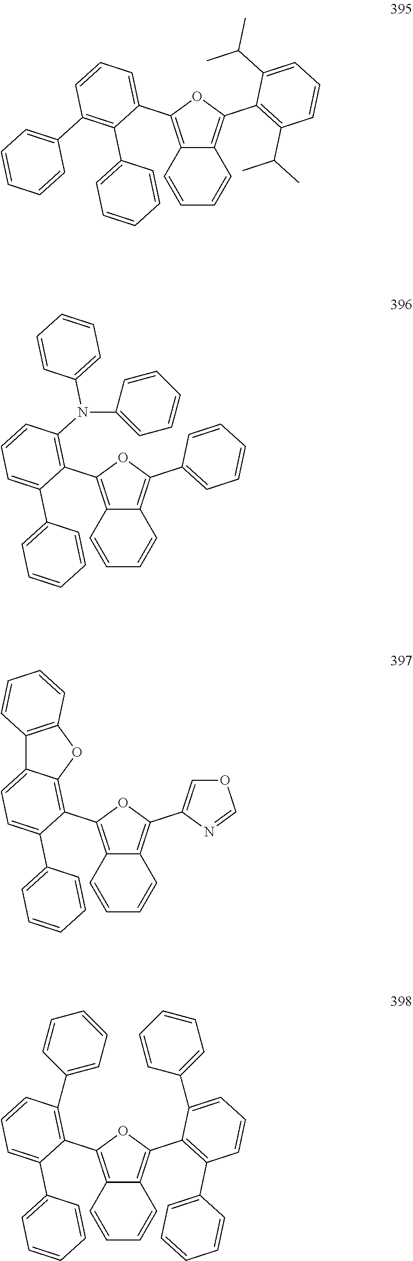 [SMILES: CC(C)c1cccc(C(C)C)c1-c1oc(-c2cccc(-c3ccccc3)c2-c2ccccc2)c2ccccc12 ; c1ccc(-c2cccc(N(c3ccccc3)c3ccccc3)c2-c2oc(-c3ccccc3)c3ccccc23)cc1 ; c1ccc(-c2ccc3c(oc4ccccc43)c2-c2oc(-c3cocn3)c3ccccc23)cc1 ; c1ccc(-c2cccc(-c3ccccc3)c2-c2oc(-c3c(-c4ccccc4)cccc3-c3ccccc3)c3ccccc23)cc1]

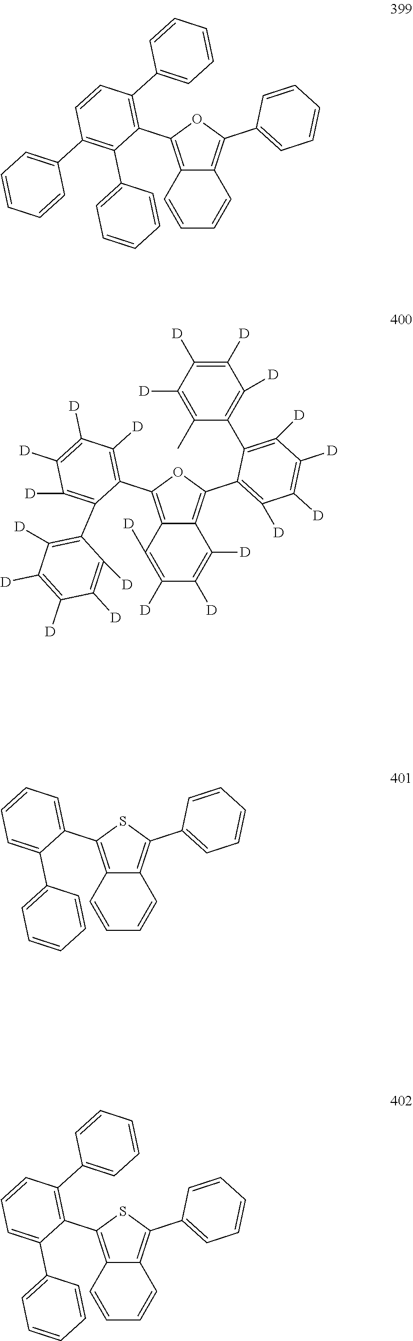 [SMILES: c1ccc(-c2ccc(-c3ccccc3)c(-c3oc(-c4ccccc4)c4ccccc34)c2-c2ccccc2)cc1 ; [2H]c1c([2H])c([2H])c(-c2c([2H])c([2H])c([2H])c([2H])c2-c2oc(-c3c([2H])c([2H])c([2H])c([2H])c3-c3c([2H])c([2H])c([2H])c([2H])c3C)c3c([2H])c([2H])c([2H])c([2H])c23)c([2H])c1[2H] ; c1ccc(-c2ccccc2-c2sc(-c3ccccc3)c3ccccc23)cc1 ; c1ccc(-c2cccc(-c3ccccc3)c2-c2sc(-c3ccccc3)c3ccccc23)cc1]

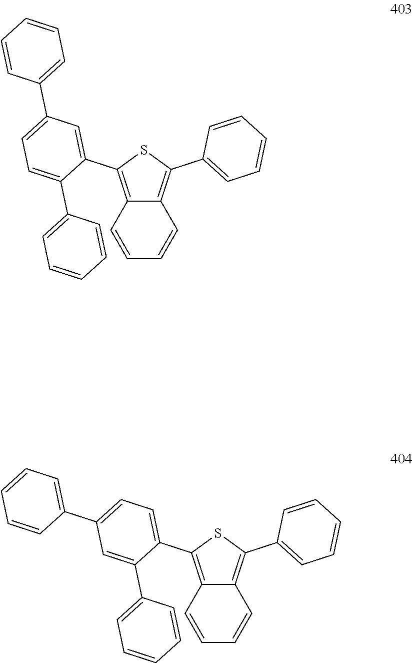 [SMILES: c1ccc(-c2ccc(-c3ccccc3)c(-c3sc(-c4ccccc4)c4ccccc34)c2)cc1 ; c1ccc(-c2ccc(-c3sc(-c4ccccc4)c4ccccc34)c(-c3ccccc3)c2)cc1]

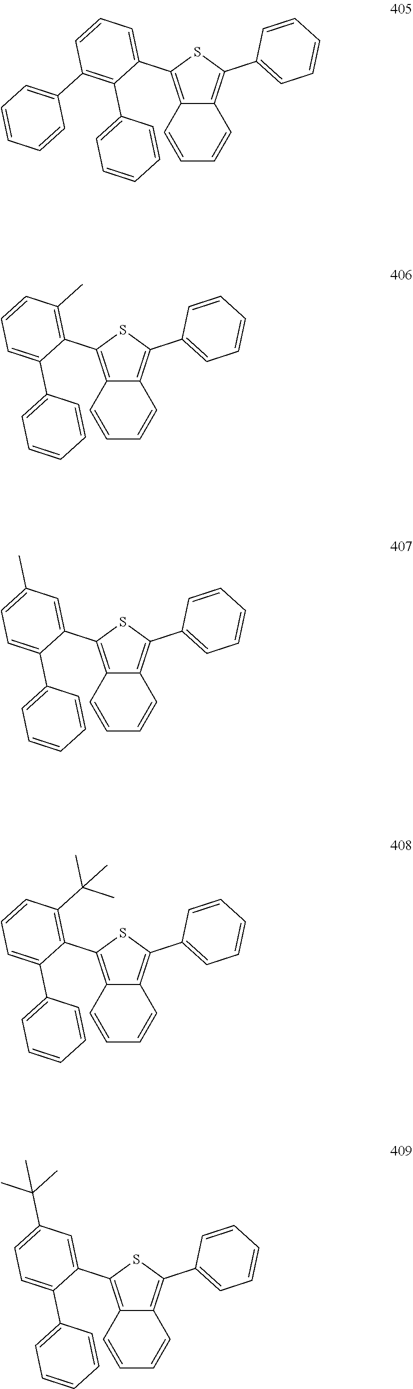 [SMILES: c1ccc(-c2cccc(-c3sc(-c4ccccc4)c4ccccc34)c2-c2ccccc2)cc1 ; Cc1cccc(-c2ccccc2)c1-c1sc(-c2ccccc2)c2ccccc12 ; Cc1ccc(-c2ccccc2)c(-c2sc(-c3ccccc3)c3ccccc23)c1 ; CC(C)(C)c1cccc(-c2ccccc2)c1-c1sc(-c2ccccc2)c2ccccc12 ; CC(C)(C)c1ccc(-c2ccccc2)c(-c2sc(-c3ccccc3)c3ccccc23)c1]

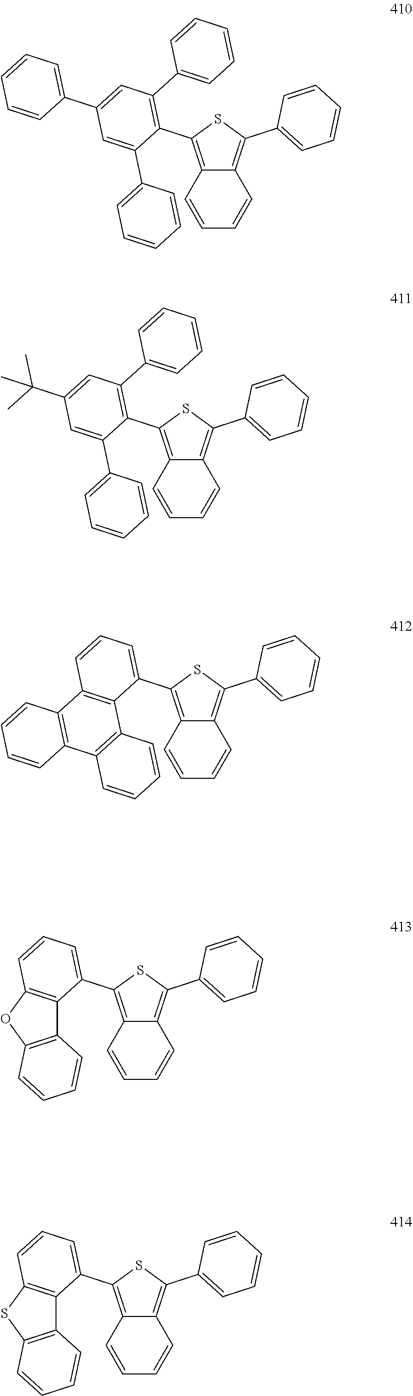 [SMILES: c1ccc(-c2cc(-c3ccccc3)c(-c3sc(-c4ccccc4)c4ccccc34)c(-c3ccccc3)c2)cc1 ; CC(C)(C)c1cc(-c2ccccc2)c(-c2sc(-c3ccccc3)c3ccccc23)c(-c2ccccc2)c1 ; c1ccc(-c2sc(-c3cccc4c5ccccc5c5ccccc5c34)c3ccccc23)cc1 ; c1ccc(-c2sc(-c3cccc4oc5ccccc5c34)c3ccccc23)cc1 ; c1ccc(-c2sc(-c3cccc4sc5ccccc5c34)c3ccccc23)cc1]

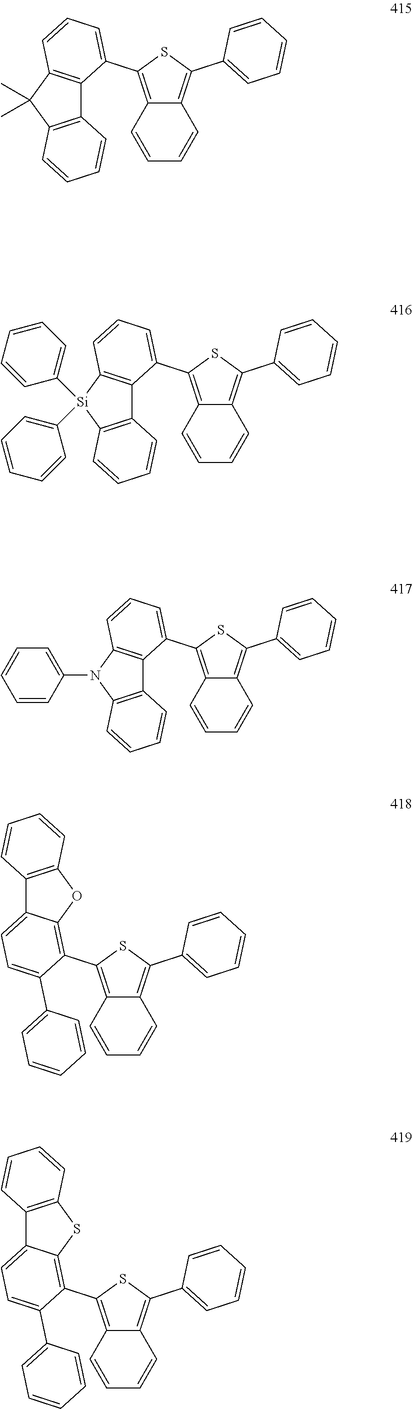 [SMILES: CC1(C)c2ccccc2-c2c(-c3sc(-c4ccccc4)c4ccccc34)cccc21 ; c1ccc(-c2sc(-c3cccc4c3-c3ccccc3[Si]4(c3ccccc3)c3ccccc3)c3ccccc23)cc1 ; c1ccc(-c2sc(-c3cccc4c3c3ccccc3n4-c3ccccc3)c3ccccc23)cc1 ; c1ccc(-c2ccc3c(oc4ccccc43)c2-c2sc(-c3ccccc3)c3ccccc23)cc1 ; c1ccc(-c2ccc3c(sc4ccccc43)c2-c2sc(-c3ccccc3)c3ccccc23)cc1]

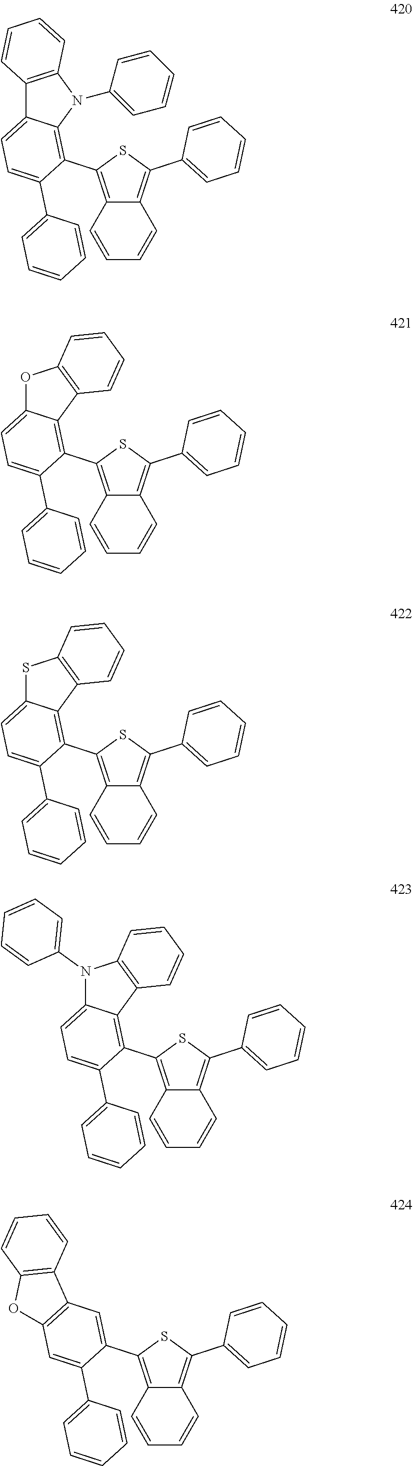

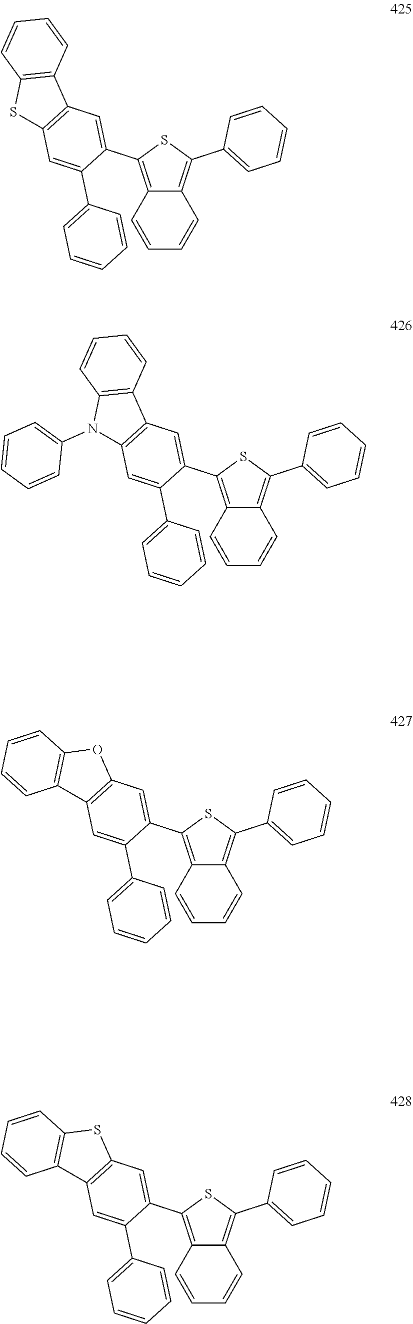 [SMILES: c1ccc(-c2cc3sc4ccccc4c3cc2-c2sc(-c3ccccc3)c3ccccc23)cc1 ; c1ccc(-c2cc3c(cc2-c2sc(-c4ccccc4)c4ccccc24)c2ccccc2n3-c2ccccc2)cc1 ; c1ccc(-c2cc3c(cc2-c2sc(-c4ccccc4)c4ccccc24)oc2ccccc23)cc1 ; c1ccc(-c2cc3c(cc2-c2sc(-c4ccccc4)c4ccccc24)sc2ccccc23)cc1]

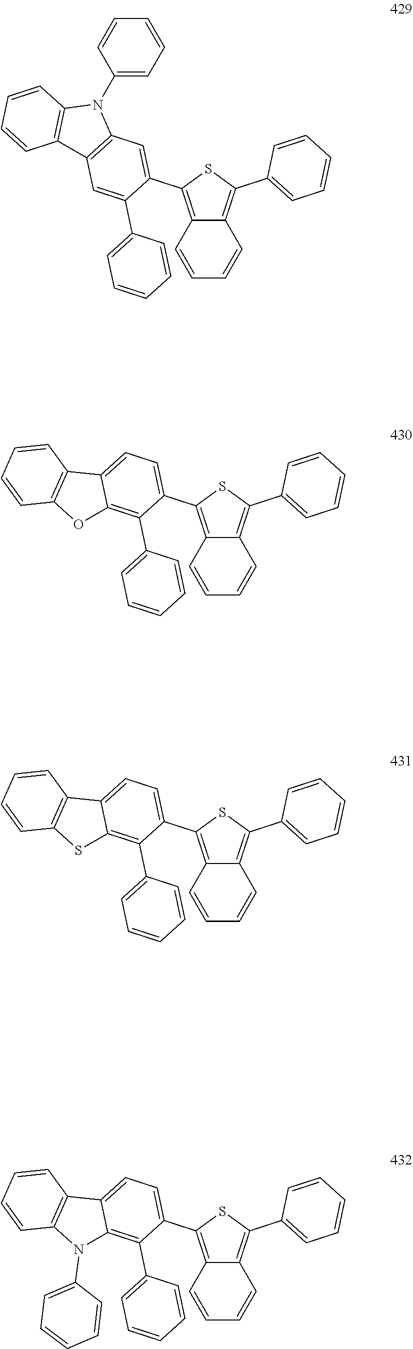 [SMILES: c1ccc(-c2cc3c4ccccc4n(-c4ccccc4)c3cc2-c2sc(-c3ccccc3)c3ccccc23)cc1 ; c1ccc(-c2sc(-c3ccc4c(oc5ccccc54)c3-c3ccccc3)c3ccccc23)cc1 ; c1ccc(-c2sc(-c3ccc4c(sc5ccccc54)c3-c3ccccc3)c3ccccc23)cc1 ; c1ccc(-c2sc(-c3ccc4c5ccccc5n(-c5ccccc5)c4c3-c3ccccc3)c3ccccc23)cc1]

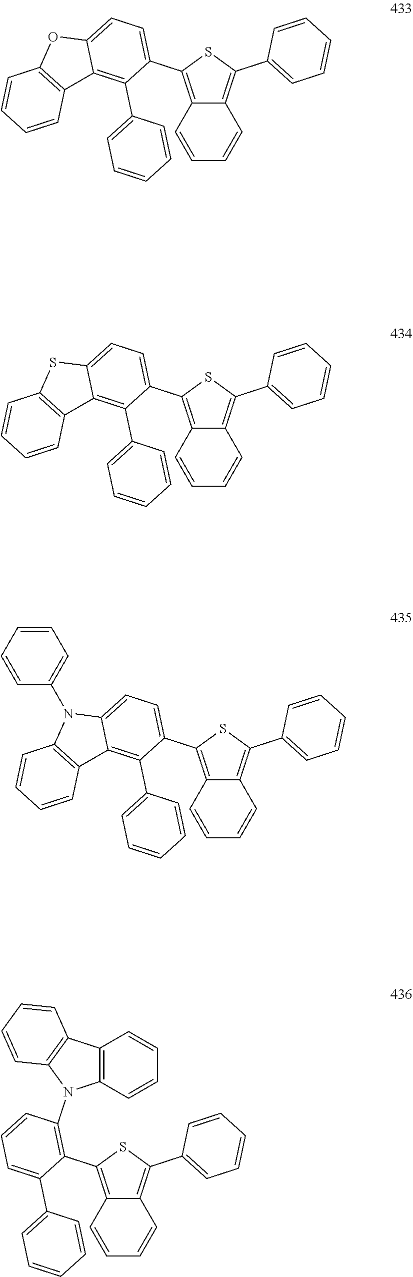 [SMILES: c1ccc(-c2sc(-c3ccc4oc5ccccc5c4c3-c3ccccc3)c3ccccc23)cc1 ; c1ccc(-c2sc(-c3ccc4sc5ccccc5c4c3-c3ccccc3)c3ccccc23)cc1 ; c1ccc(-c2sc(-c3ccc4c(c3-c3ccccc3)c3ccccc3n4-c3ccccc3)c3ccccc23)cc1 ; c1ccc(-c2cccc(-n3c4ccccc4c4ccccc43)c2-c2sc(-c3ccccc3)c3ccccc23)cc1]

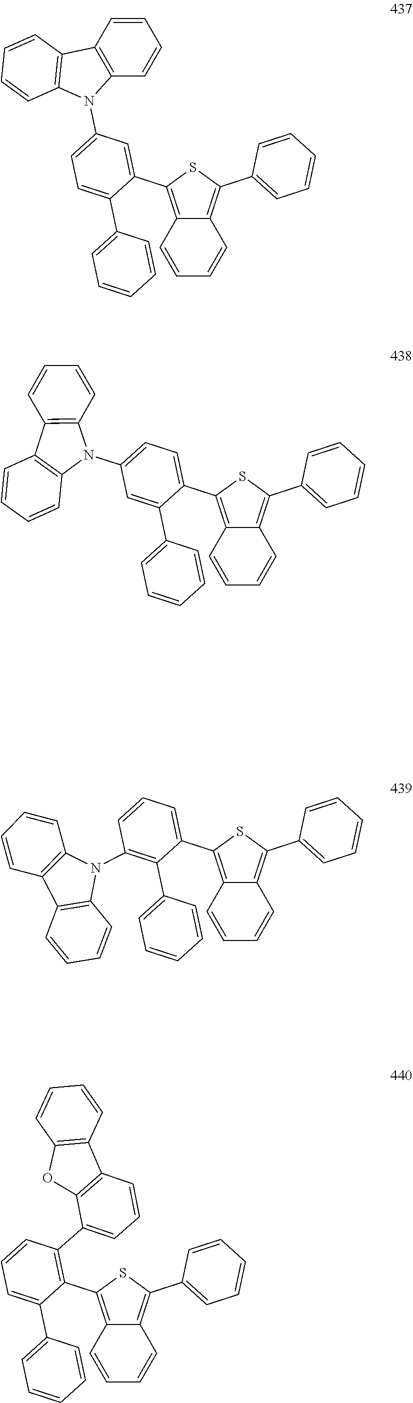 [SMILES: c1ccc(-c2ccc(-n3c4ccccc4c4ccccc43)cc2-c2sc(-c3ccccc3)c3ccccc23)cc1 ; c1ccc(-c2cc(-n3c4ccccc4c4ccccc43)ccc2-c2sc(-c3ccccc3)c3ccccc23)cc1 ; c1ccc(-c2c(-c3sc(-c4ccccc4)c4ccccc34)cccc2-n2c3ccccc3c3ccccc32)cc1 ; c1ccc(-c2cccc(-c3cccc4c3oc3ccccc34)c2-c2sc(-c3ccccc3)c3ccccc23)cc1]

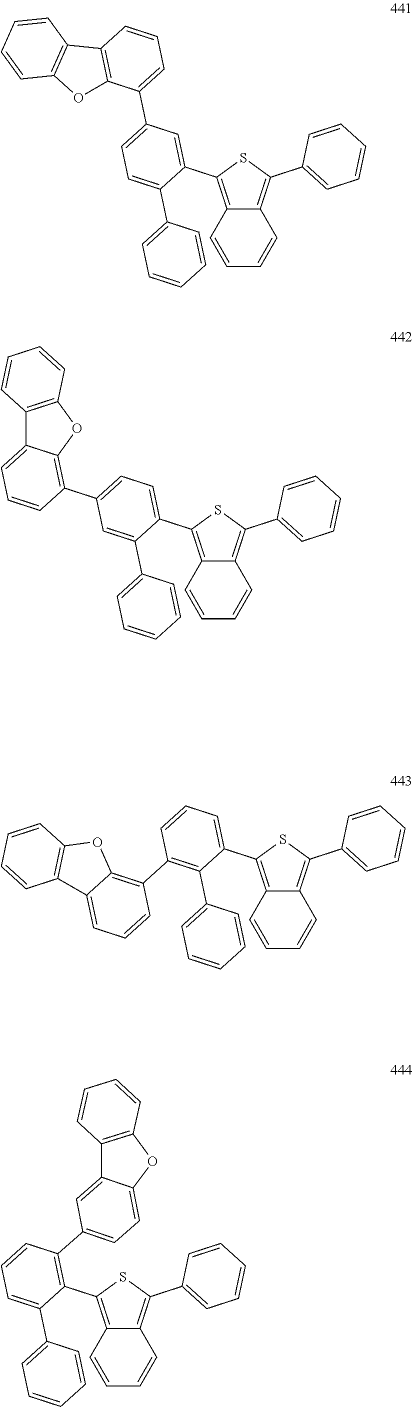 [SMILES: c1ccc(-c2ccc(-c3cccc4c3oc3ccccc34)cc2-c2sc(-c3ccccc3)c3ccccc23)cc1 ; c1ccc(-c2cc(-c3cccc4c3oc3ccccc34)ccc2-c2sc(-c3ccccc3)c3ccccc23)cc1 ; c1ccc(-c2c(-c3sc(-c4ccccc4)c4ccccc34)cccc2-c2cccc3c2oc2ccccc23)cc1 ; c1ccc(-c2cccc(-c3ccc4oc5ccccc5c4c3)c2-c2sc(-c3ccccc3)c3ccccc23)cc1]

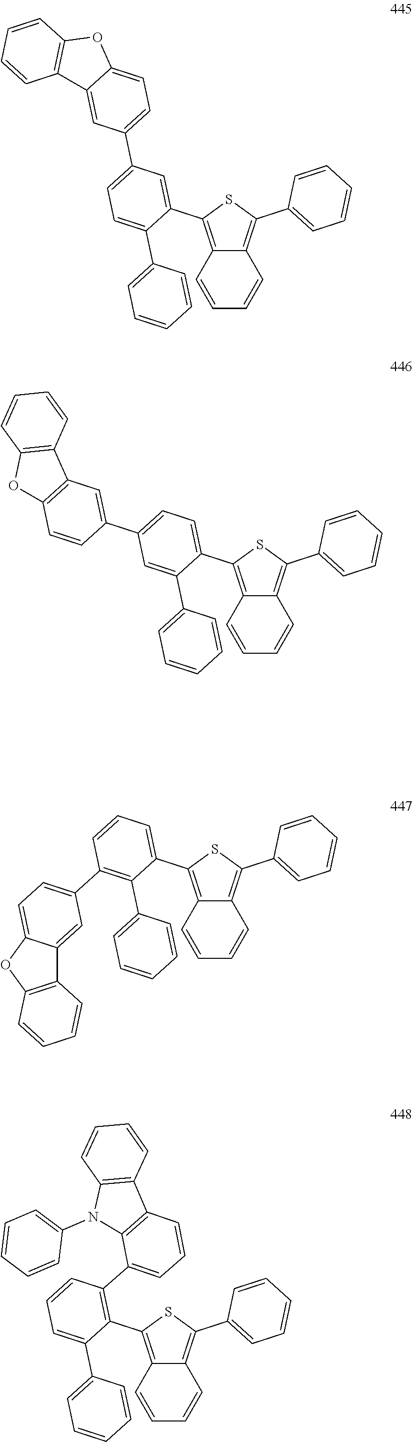 [SMILES: c1ccc(-c2ccc(-c3ccc4oc5ccccc5c4c3)cc2-c2sc(-c3ccccc3)c3ccccc23)cc1 ; c1ccc(-c2cc(-c3ccc4oc5ccccc5c4c3)ccc2-c2sc(-c3ccccc3)c3ccccc23)cc1 ; c1ccc(-c2c(-c3ccc4oc5ccccc5c4c3)cccc2-c2sc(-c3ccccc3)c3ccccc23)cc1 ; c1ccc(-c2cccc(-c3cccc4c5ccccc5n(-c5ccccc5)c34)c2-c2sc(-c3ccccc3)c3ccccc23)cc1]

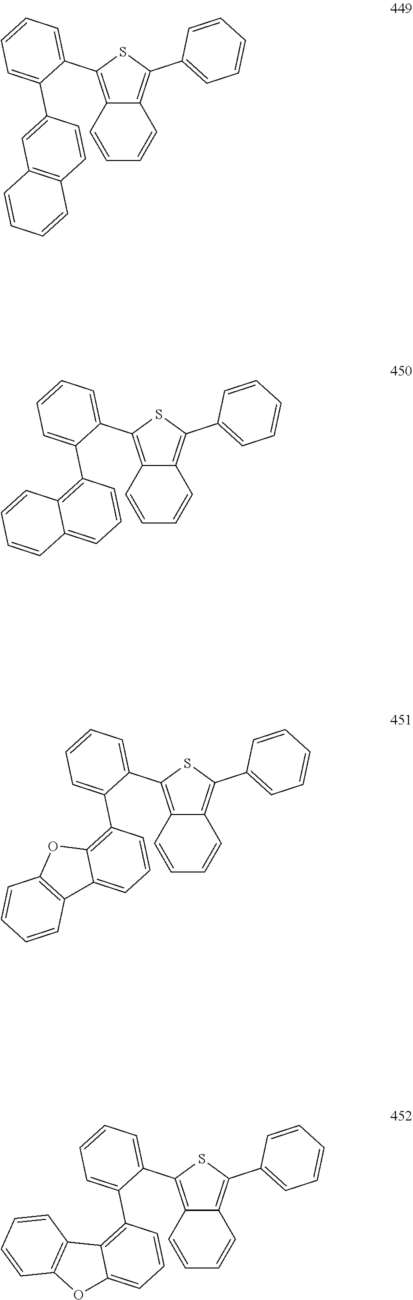 [SMILES: c1ccc(-c2sc(-c3ccccc3-c3ccc4ccccc4c3)c3ccccc23)cc1 ; c1ccc(-c2sc(-c3ccccc3-c3cccc4ccccc34)c3ccccc23)cc1 ; c1ccc(-c2sc(-c3ccccc3-c3cccc4c3oc3ccccc34)c3ccccc23)cc1 ; c1ccc(-c2sc(-c3ccccc3-c3cccc4oc5ccccc5c34)c3ccccc23)cc1]

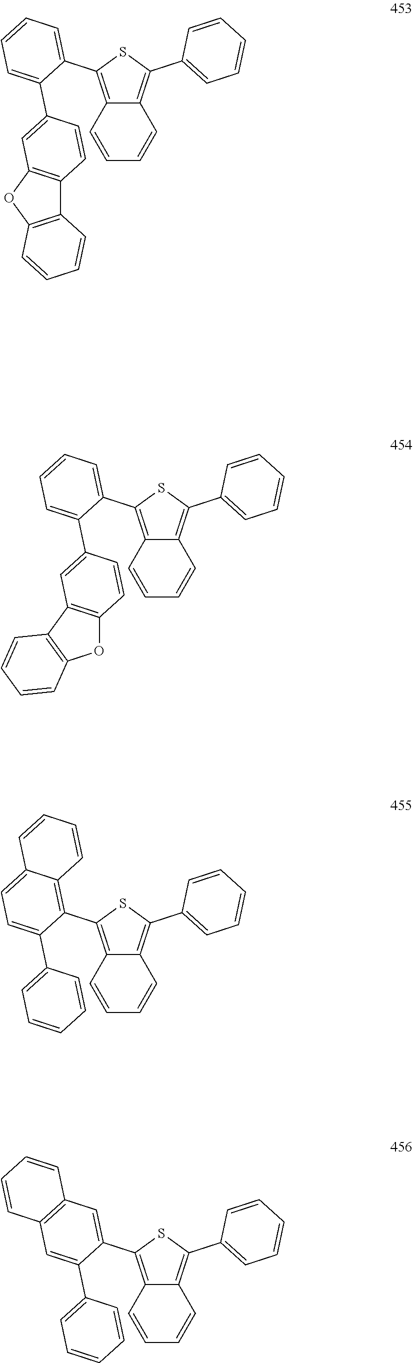 [SMILES: c1ccc(-c2sc(-c3ccccc3-c3ccc4c(c3)oc3ccccc34)c3ccccc23)cc1 ; c1ccc(-c2sc(-c3ccccc3-c3ccc4oc5ccccc5c4c3)c3ccccc23)cc1 ; c1ccc(-c2ccc3ccccc3c2-c2sc(-c3ccccc3)c3ccccc23)cc1 ; c1ccc(-c2cc3ccccc3cc2-c2sc(-c3ccccc3)c3ccccc23)cc1]

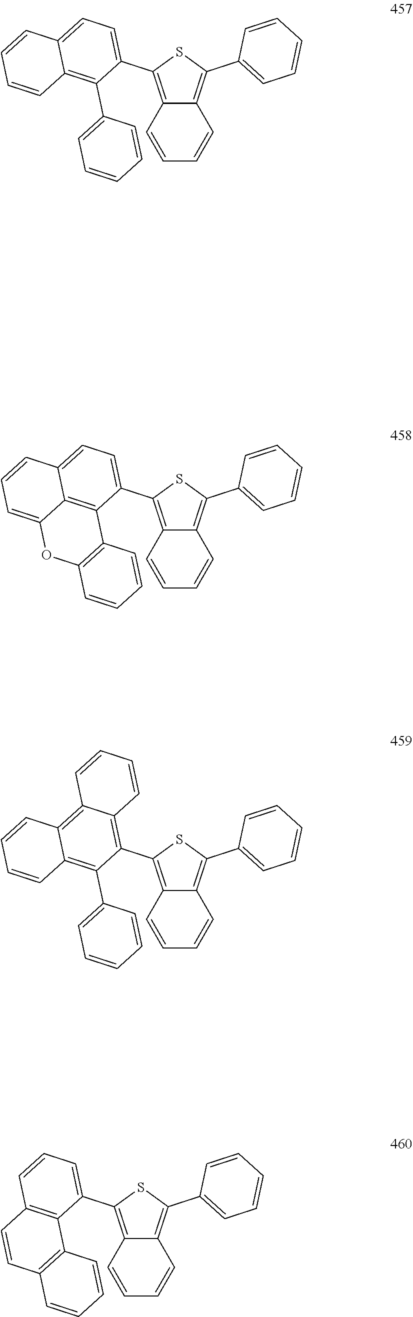 [SMILES: c1ccc(-c2c(-c3sc(-c4ccccc4)c4ccccc34)ccc3ccccc23)cc1 ; c1ccc(-c2sc(-c3ccc4cccc5c4c3-c3ccccc3O5)c3ccccc23)cc1 ; c1ccc(-c2sc(-c3c(-c4ccccc4)c4ccccc4c4ccccc34)c3ccccc23)cc1 ; c1ccc(-c2sc(-c3cccc4ccc5ccccc5c34)c3ccccc23)cc1]

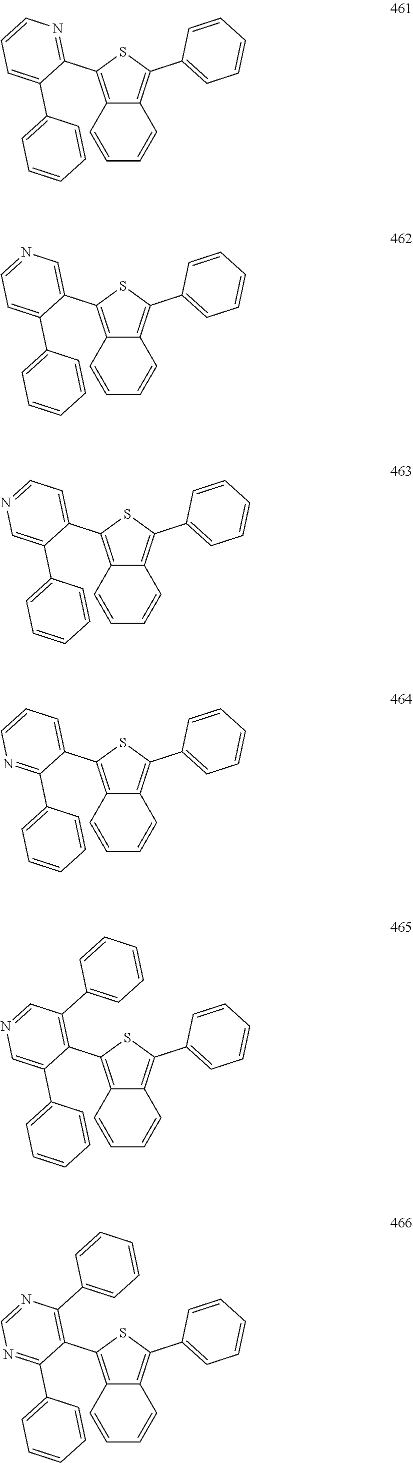 [SMILES: c1ccc(-c2cccnc2-c2sc(-c3ccccc3)c3ccccc23)cc1 ; c1ccc(-c2ccncc2-c2sc(-c3ccccc3)c3ccccc23)cc1 ; c1ccc(-c2cnccc2-c2sc(-c3ccccc3)c3ccccc23)cc1 ; c1ccc(-c2ncccc2-c2sc(-c3ccccc3)c3ccccc23)cc1 ; c1ccc(-c2cncc(-c3ccccc3)c2-c2sc(-c3ccccc3)c3ccccc23)cc1 ; c1ccc(-c2ncnc(-c3ccccc3)c2-c2sc(-c3ccccc3)c3ccccc23)cc1]

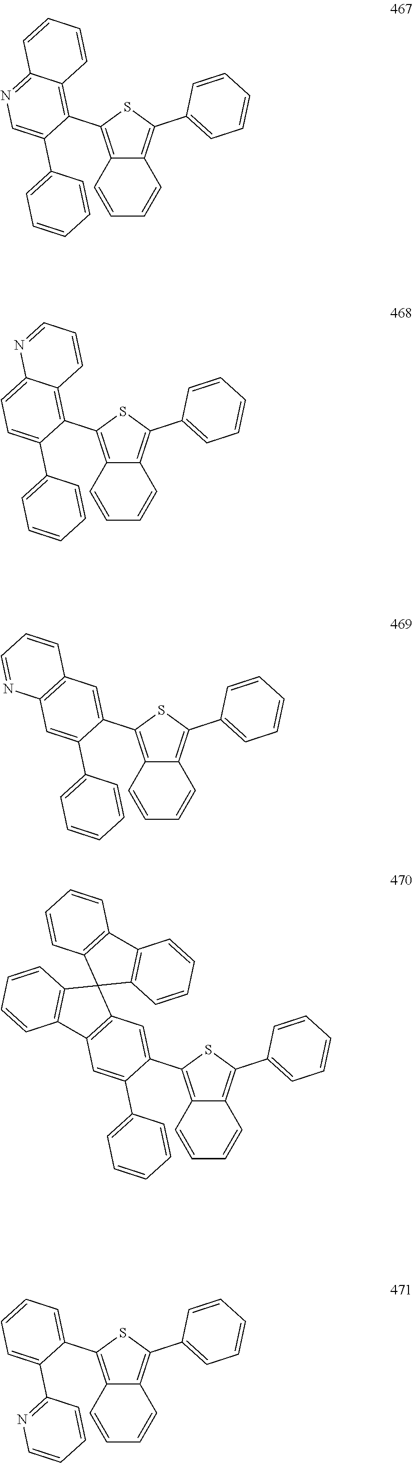 [SMILES: c1ccc(-c2cnc3ccccc3c2-c2sc(-c3ccccc3)c3ccccc23)cc1 ; c1ccc(-c2ccc3ncccc3c2-c2sc(-c3ccccc3)c3ccccc23)cc1 ; c1ccc(-c2cc3ncccc3cc2-c2sc(-c3ccccc3)c3ccccc23)cc1 ; c1ccc(-c2cc3c(cc2-c2sc(-c4ccccc4)c4ccccc24)C2(c4ccccc4-c4ccccc42)c2ccccc2-3)cc1 ; c1ccc(-c2sc(-c3ccccc3-c3ccccn3)c3ccccc23)cc1]

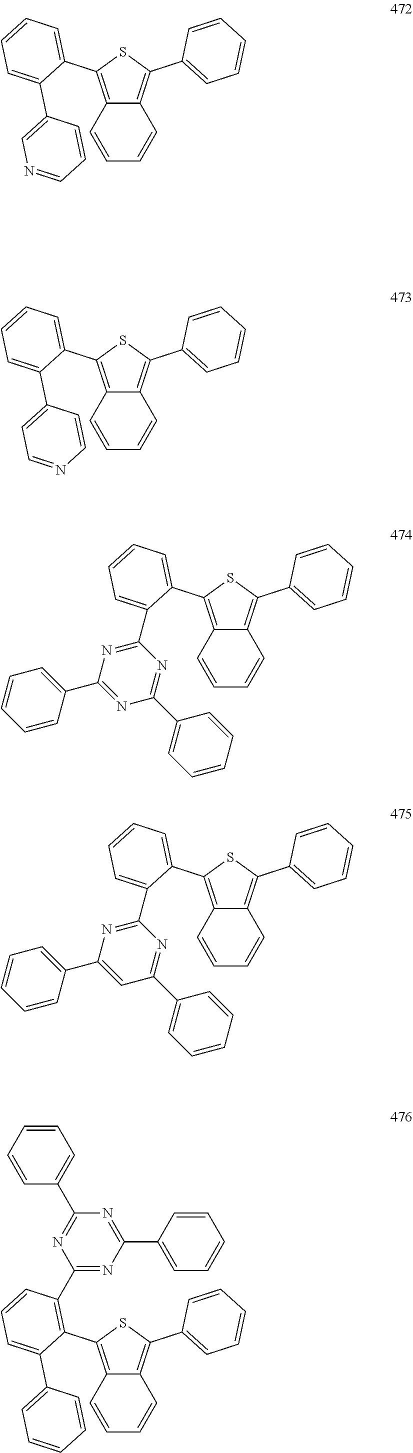 [SMILES: c1ccc(-c2sc(-c3ccccc3-c3cccnc3)c3ccccc23)cc1 ; c1ccc(-c2sc(-c3ccccc3-c3ccncc3)c3ccccc23)cc1 ; c1ccc(-c2nc(-c3ccccc3)nc(-c3ccccc3-c3sc(-c4ccccc4)c4ccccc34)n2)cc1 ; c1ccc(-c2cc(-c3ccccc3)nc(-c3ccccc3-c3sc(-c4ccccc4)c4ccccc34)n2)cc1 ; c1ccc(-c2nc(-c3ccccc3)nc(-c3cccc(-c4ccccc4)c3-c3sc(-c4ccccc4)c4ccccc34)n2)cc1]

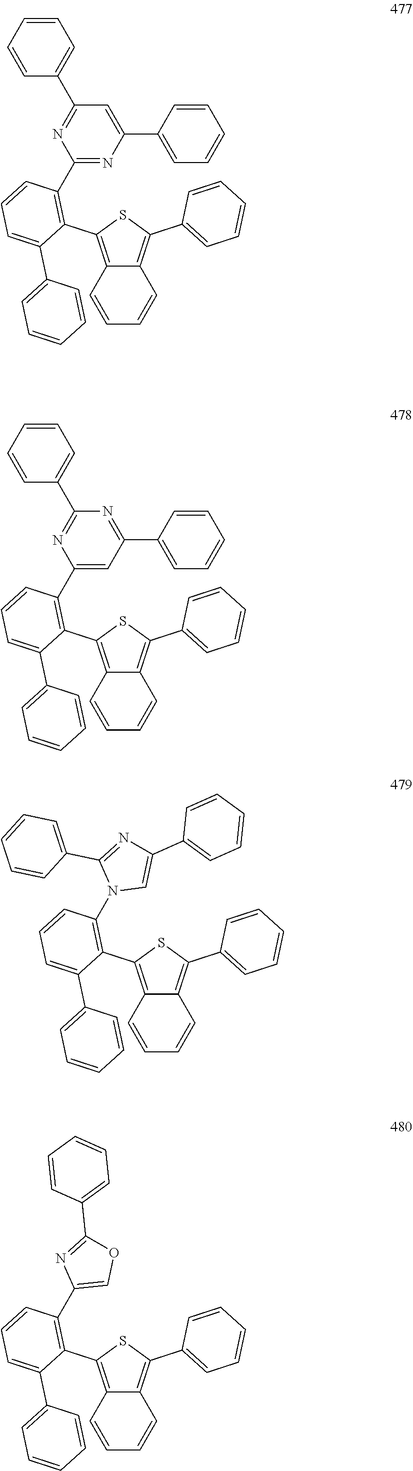 [SMILES: c1ccc(-c2cc(-c3ccccc3)nc(-c3cccc(-c4ccccc4)c3-c3sc(-c4ccccc4)c4ccccc34)n2)cc1 ; c1ccc(-c2cc(-c3cccc(-c4ccccc4)c3-c3sc(-c4ccccc4)c4ccccc34)nc(-c3ccccc3)n2)cc1 ; c1ccc(-c2cn(-c3cccc(-c4ccccc4)c3-c3sc(-c4ccccc4)c4ccccc34)c(-c3ccccc3)n2)cc1 ; c1ccc(-c2nc(-c3cccc(-c4ccccc4)c3-c3sc(-c4ccccc4)c4ccccc34)co2)cc1]

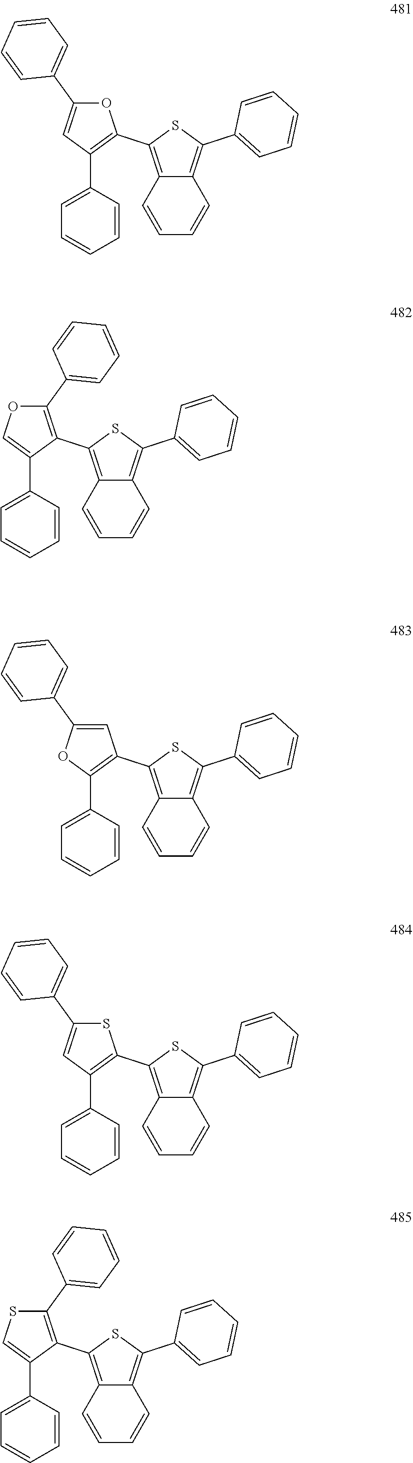 [SMILES: c1ccc(-c2cc(-c3ccccc3)c(-c3sc(-c4ccccc4)c4ccccc34)o2)cc1 ; c1ccc(-c2coc(-c3ccccc3)c2-c2sc(-c3ccccc3)c3ccccc23)cc1 ; c1ccc(-c2cc(-c3sc(-c4ccccc4)c4ccccc34)c(-c3ccccc3)o2)cc1 ; c1ccc(-c2cc(-c3ccccc3)c(-c3sc(-c4ccccc4)c4ccccc34)s2)cc1 ; c1ccc(-c2csc(-c3ccccc3)c2-c2sc(-c3ccccc3)c3ccccc23)cc1]

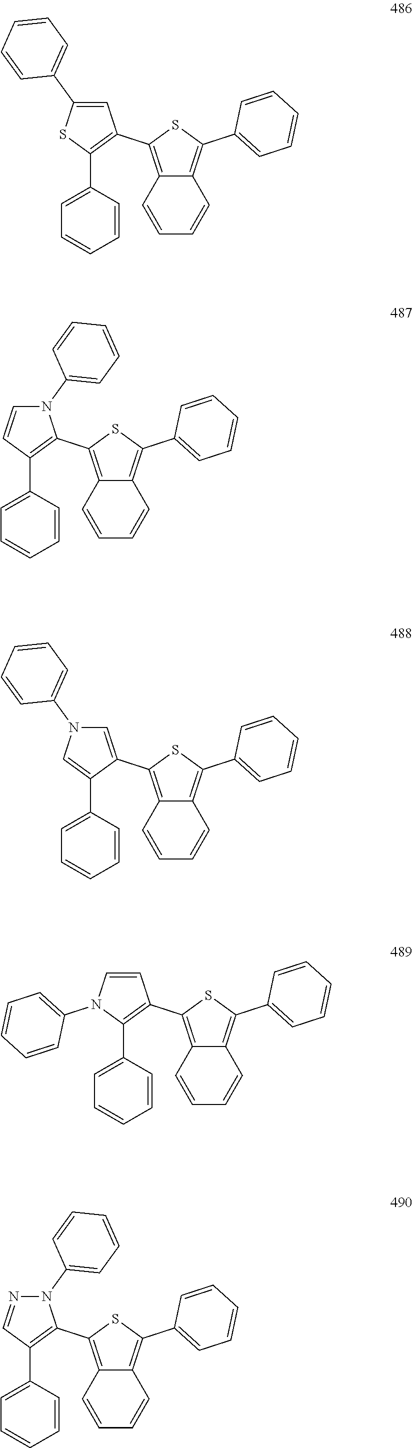 [SMILES: c1ccc(-c2cc(-c3sc(-c4ccccc4)c4ccccc34)c(-c3ccccc3)s2)cc1 ; c1ccc(-c2ccn(-c3ccccc3)c2-c2sc(-c3ccccc3)c3ccccc23)cc1 ; c1ccc(-c2cn(-c3ccccc3)cc2-c2sc(-c3ccccc3)c3ccccc23)cc1 ; c1ccc(-c2sc(-c3ccn(-c4ccccc4)c3-c3ccccc3)c3ccccc23)cc1 ; c1ccc(-c2cnn(-c3ccccc3)c2-c2sc(-c3ccccc3)c3ccccc23)cc1]

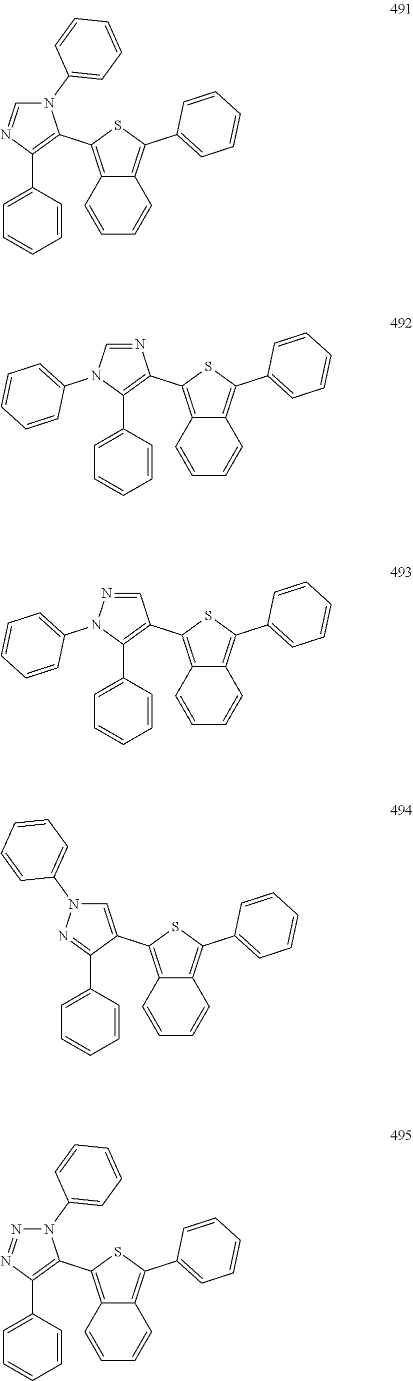 [SMILES: c1ccc(-c2ncn(-c3ccccc3)c2-c2sc(-c3ccccc3)c3ccccc23)cc1 ; c1ccc(-c2sc(-c3ncn(-c4ccccc4)c3-c3ccccc3)c3ccccc23)cc1 ; c1ccc(-c2sc(-c3cnn(-c4ccccc4)c3-c3ccccc3)c3ccccc23)cc1 ; c1ccc(-c2nn(-c3ccccc3)cc2-c2sc(-c3ccccc3)c3ccccc23)cc1 ; c1ccc(-c2nnn(-c3ccccc3)c2-c2sc(-c3ccccc3)c3ccccc23)cc1]

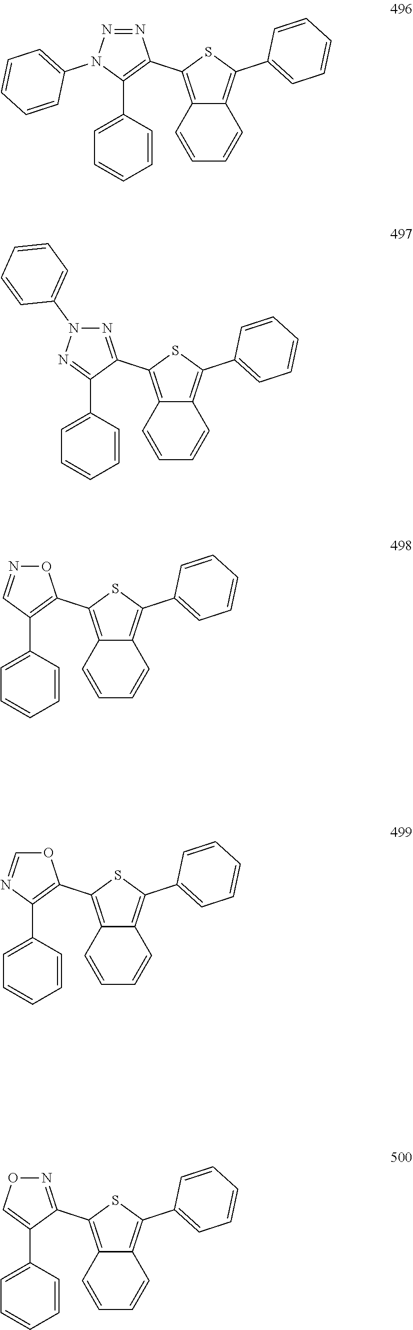 [SMILES: c1ccc(-c2sc(-c3nnn(-c4ccccc4)c3-c3ccccc3)c3ccccc23)cc1 ; c1ccc(-c2nn(-c3ccccc3)nc2-c2sc(-c3ccccc3)c3ccccc23)cc1 ; c1ccc(-c2cnoc2-c2sc(-c3ccccc3)c3ccccc23)cc1 ; c1ccc(-c2ncoc2-c2sc(-c3ccccc3)c3ccccc23)cc1 ; c1ccc(-c2conc2-c2sc(-c3ccccc3)c3ccccc23)cc1]

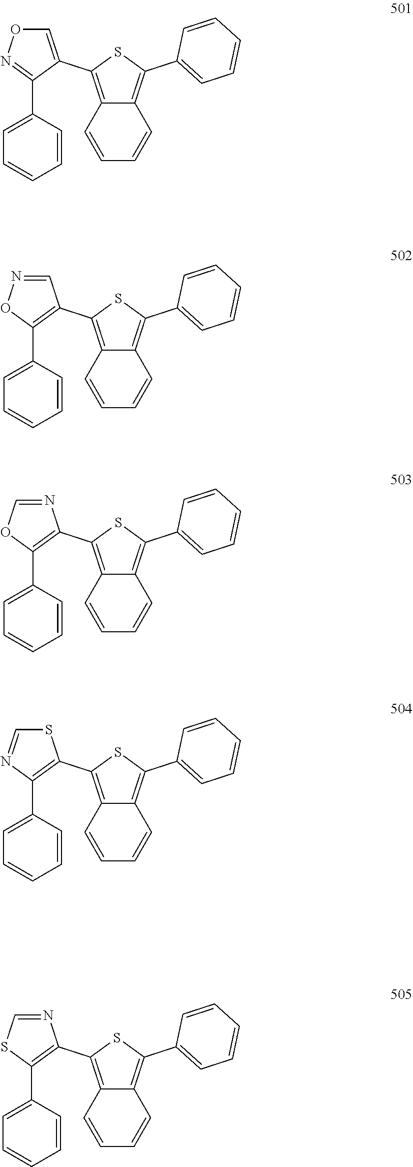 [SMILES: c1ccc(-c2nocc2-c2sc(-c3ccccc3)c3ccccc23)cc1 ; c1ccc(-c2oncc2-c2sc(-c3ccccc3)c3ccccc23)cc1 ; c1ccc(-c2ocnc2-c2sc(-c3ccccc3)c3ccccc23)cc1 ; c1ccc(-c2ncsc2-c2sc(-c3ccccc3)c3ccccc23)cc1 ; c1ccc(-c2scnc2-c2sc(-c3ccccc3)c3ccccc23)cc1]

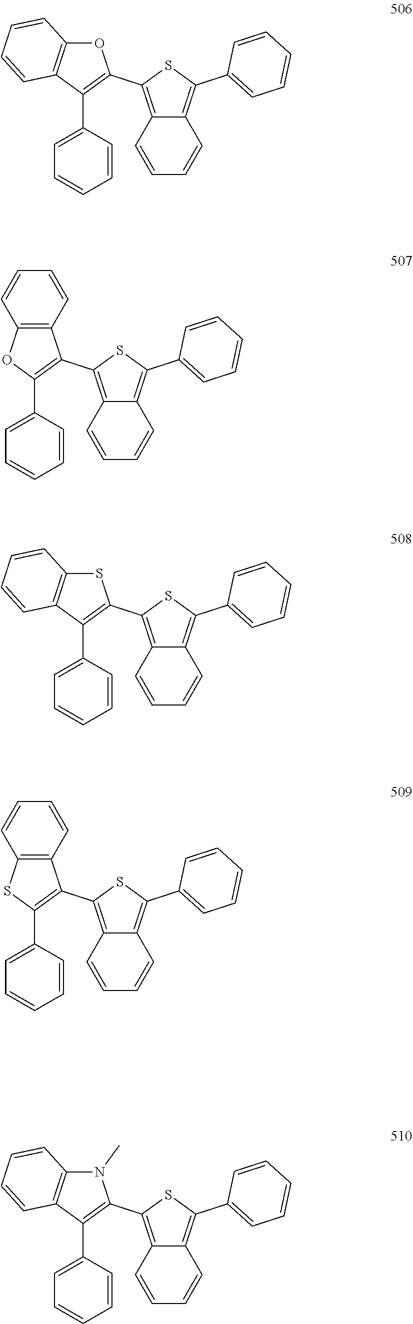 [SMILES: c1ccc(-c2c(-c3sc(-c4ccccc4)c4ccccc34)oc3ccccc23)cc1 ; c1ccc(-c2oc3ccccc3c2-c2sc(-c3ccccc3)c3ccccc23)cc1 ; c1ccc(-c2c(-c3sc(-c4ccccc4)c4ccccc34)sc3ccccc23)cc1 ; c1ccc(-c2sc3ccccc3c2-c2sc(-c3ccccc3)c3ccccc23)cc1 ; Cn1c(-c2sc(-c3ccccc3)c3ccccc23)c(-c2ccccc2)c2ccccc21]

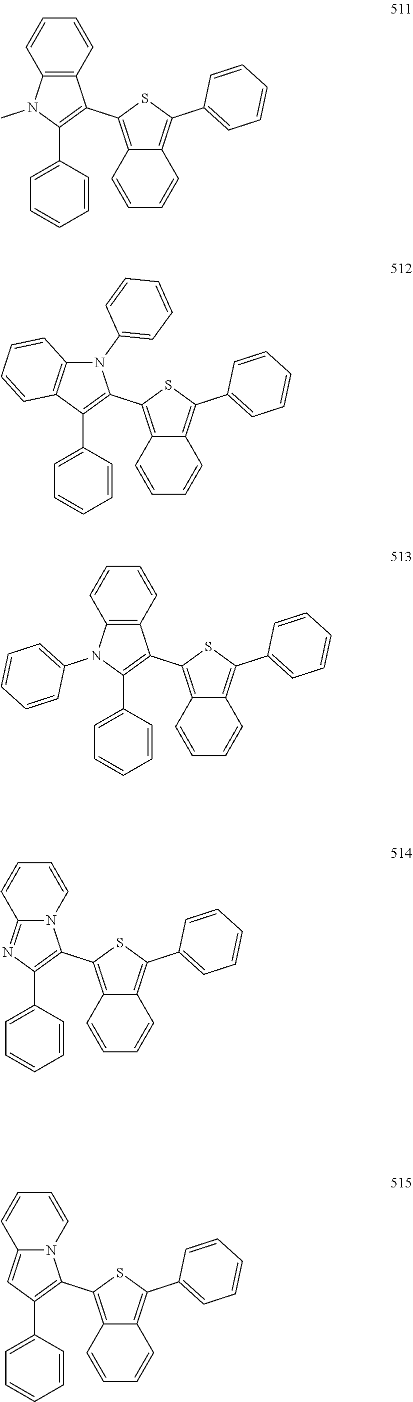 [SMILES: Cn1c(-c2ccccc2)c(-c2sc(-c3ccccc3)c3ccccc23)c2ccccc21 ; c1ccc(-c2sc(-c3c(-c4ccccc4)c4ccccc4n3-c3ccccc3)c3ccccc23)cc1 ; c1ccc(-c2sc(-c3c(-c4ccccc4)n(-c4ccccc4)c4ccccc34)c3ccccc23)cc1 ; c1ccc(-c2nc3ccccn3c2-c2sc(-c3ccccc3)c3ccccc23)cc1 ; c1ccc(-c2cc3ccccn3c2-c2sc(-c3ccccc3)c3ccccc23)cc1]

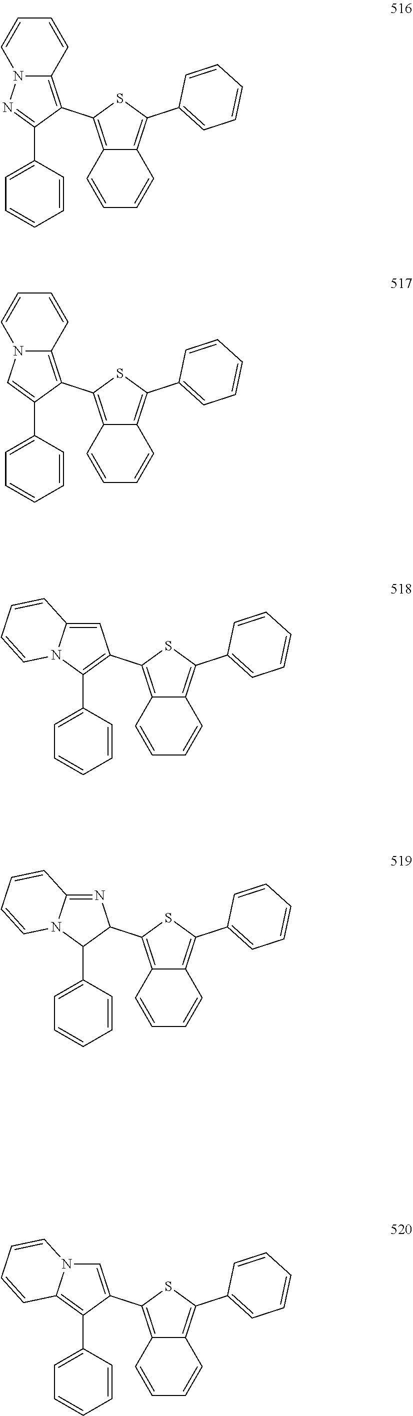 [SMILES: c1ccc(-c2nn3ccccc3c2-c2sc(-c3ccccc3)c3ccccc23)cc1 ; c1ccc(-c2cn3ccccc3c2-c2sc(-c3ccccc3)c3ccccc23)cc1 ; c1ccc(-c2sc(-c3cc4ccccn4c3-c3ccccc3)c3ccccc23)cc1 ; C1=CC2=NC(c3sc(-c4ccccc4)c4ccccc34)C(c3ccccc3)N2C=C1 ; c1ccc(-c2sc(-c3cn4ccccc4c3-c3ccccc3)c3ccccc23)cc1]

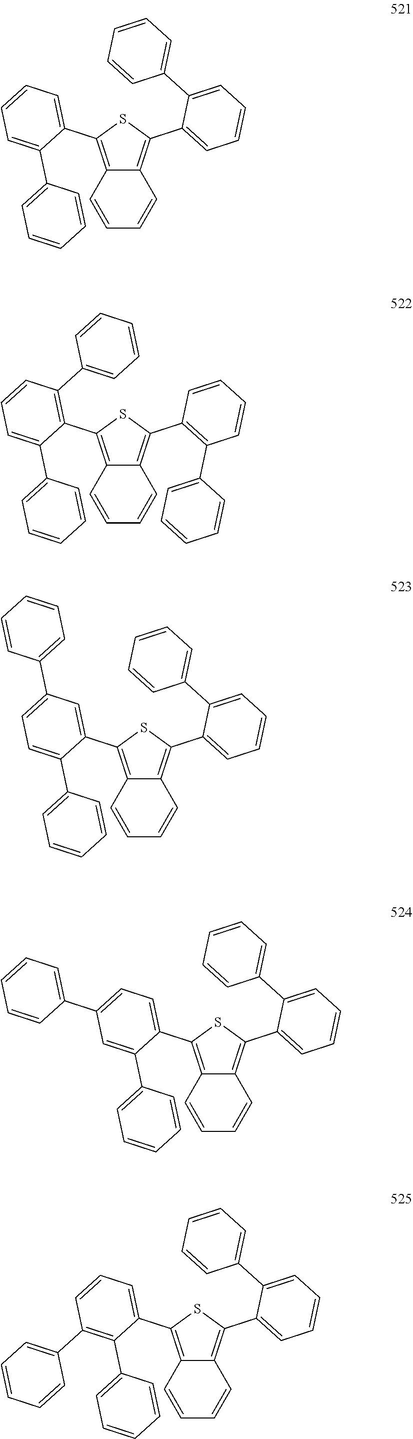 [SMILES: c1ccc(-c2ccccc2-c2sc(-c3ccccc3-c3ccccc3)c3ccccc23)cc1 ; c1ccc(-c2ccccc2-c2sc(-c3c(-c4ccccc4)cccc3-c3ccccc3)c3ccccc23)cc1 ; c1ccc(-c2ccc(-c3ccccc3)c(-c3sc(-c4ccccc4-c4ccccc4)c4ccccc34)c2)cc1 ; c1ccc(-c2ccc(-c3sc(-c4ccccc4-c4ccccc4)c4ccccc34)c(-c3ccccc3)c2)cc1 ; c1ccc(-c2ccccc2-c2sc(-c3cccc(-c4ccccc4)c3-c3ccccc3)c3ccccc23)cc1]

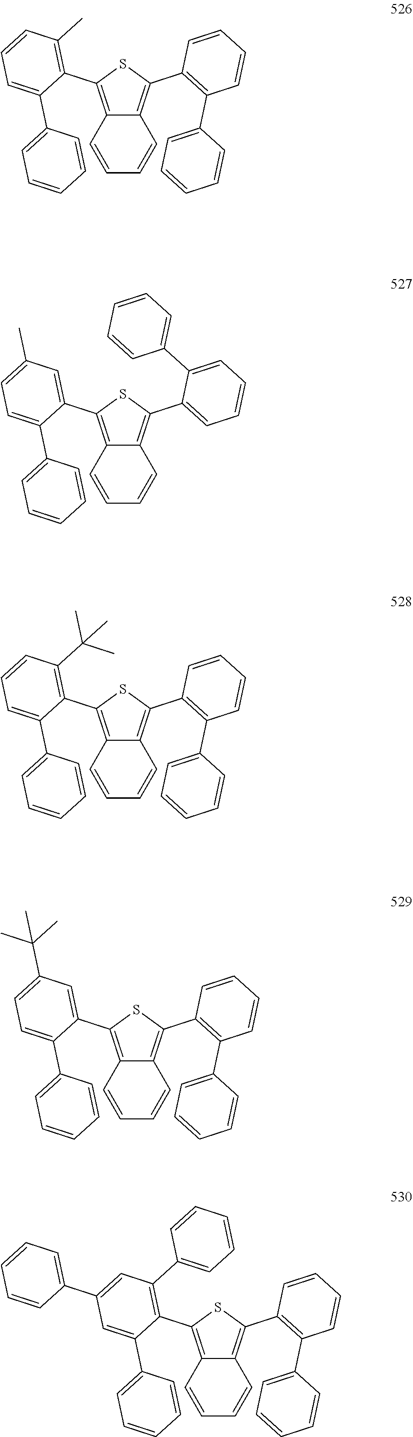 [SMILES: Cc1cccc(-c2ccccc2)c1-c1sc(-c2ccccc2-c2ccccc2)c2ccccc12 ; Cc1ccc(-c2ccccc2)c(-c2sc(-c3ccccc3-c3ccccc3)c3ccccc23)c1 ; CC(C)(C)c1cccc(-c2ccccc2)c1-c1sc(-c2ccccc2-c2ccccc2)c2ccccc12 ; CC(C)(C)c1ccc(-c2ccccc2)c(-c2sc(-c3ccccc3-c3ccccc3)c3ccccc23)c1 ; c1ccc(-c2cc(-c3ccccc3)c(-c3sc(-c4ccccc4-c4ccccc4)c4ccccc34)c(-c3ccccc3)c2)cc1]

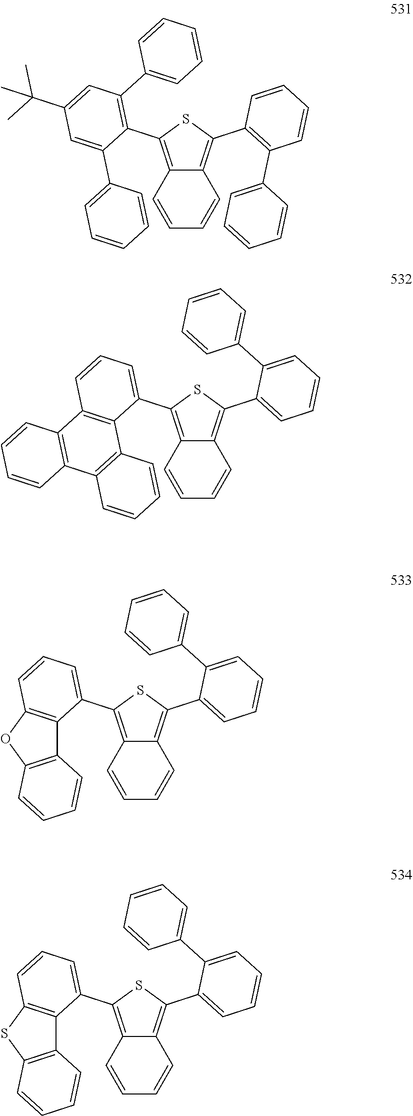 [SMILES: CC(C)(C)c1cc(-c2ccccc2)c(-c2sc(-c3ccccc3-c3ccccc3)c3ccccc23)c(-c2ccccc2)c1 ; c1ccc(-c2ccccc2-c2sc(-c3cccc4c5ccccc5c5ccccc5c34)c3ccccc23)cc1 ; c1ccc(-c2ccccc2-c2sc(-c3cccc4oc5ccccc5c34)c3ccccc23)cc1 ; c1ccc(-c2ccccc2-c2sc(-c3cccc4sc5ccccc5c34)c3ccccc23)cc1]

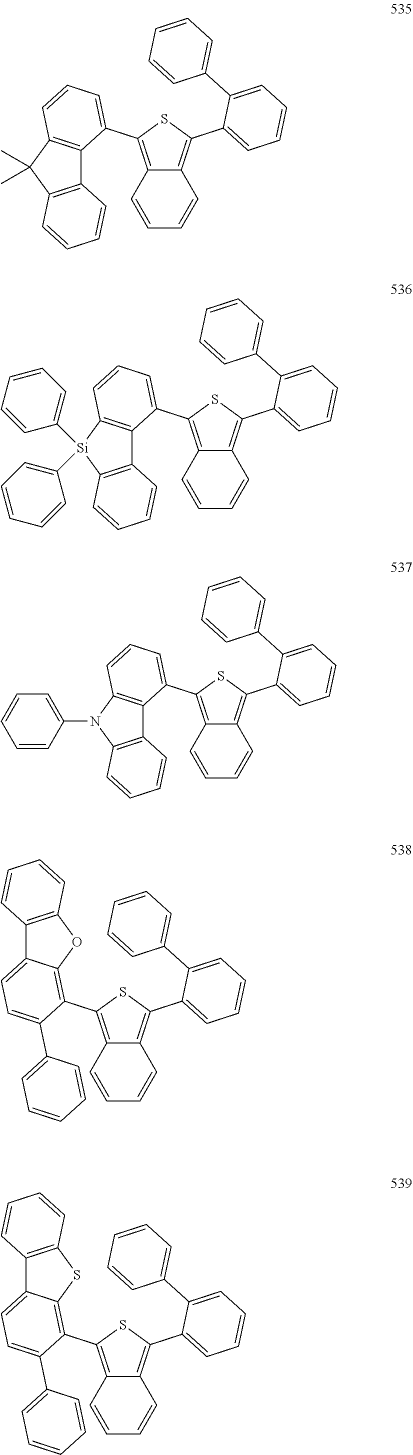 [SMILES: CC1(C)c2ccccc2-c2c(-c3sc(-c4ccccc4-c4ccccc4)c4ccccc34)cccc21 ; c1ccc(-c2ccccc2-c2sc(-c3cccc4c3-c3ccccc3[Si]4(c3ccccc3)c3ccccc3)c3ccccc23)cc1 ; c1ccc(-c2ccccc2-c2sc(-c3cccc4c3c3ccccc3n4-c3ccccc3)c3ccccc23)cc1 ; c1ccc(-c2ccccc2-c2sc(-c3c(-c4ccccc4)ccc4c3oc3ccccc34)c3ccccc23)cc1 ; c1ccc(-c2ccccc2-c2sc(-c3c(-c4ccccc4)ccc4c3sc3ccccc34)c3ccccc23)cc1]

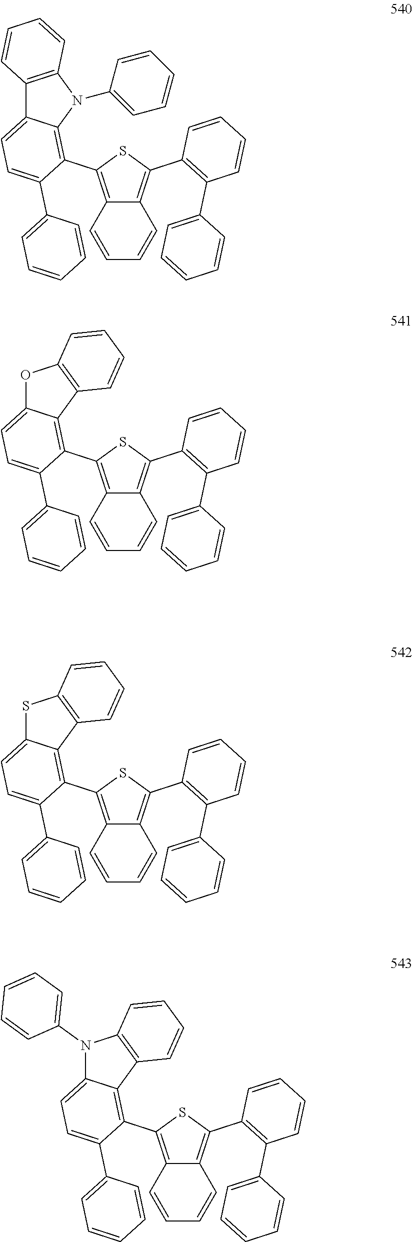 [SMILES: c1ccc(-c2ccccc2-c2sc(-c3c(-c4ccccc4)ccc4c5ccccc5n(-c5ccccc5)c34)c3ccccc23)cc1 ; c1ccc(-c2ccccc2-c2sc(-c3c(-c4ccccc4)ccc4oc5ccccc5c34)c3ccccc23)cc1 ; c1ccc(-c2ccccc2-c2sc(-c3c(-c4ccccc4)ccc4sc5ccccc5c34)c3ccccc23)cc1 ; c1ccc(-c2ccccc2-c2sc(-c3c(-c4ccccc4)ccc4c3c3ccccc3n4-c3ccccc3)c3ccccc23)cc1]

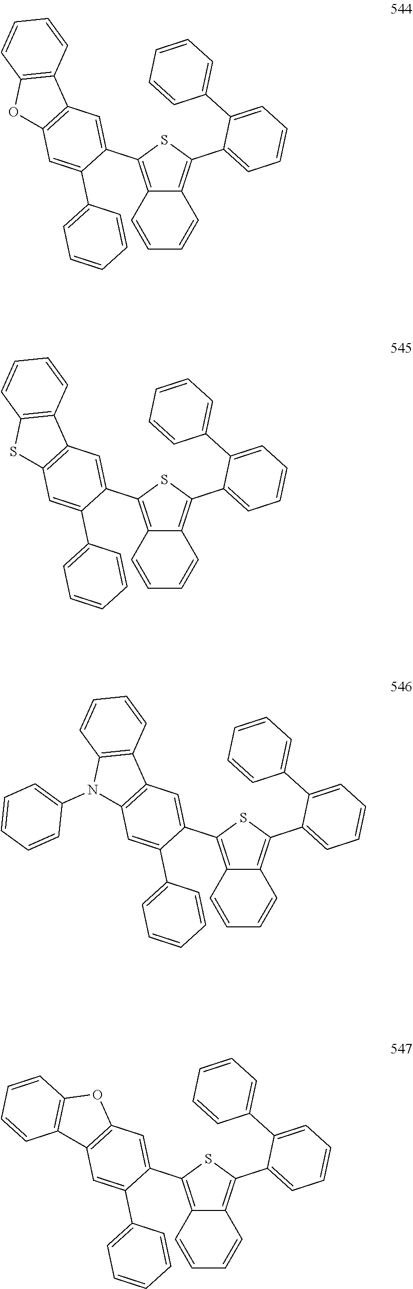 [SMILES: c1ccc(-c2ccccc2-c2sc(-c3cc4c(cc3-c3ccccc3)oc3ccccc34)c3ccccc23)cc1 ; c1ccc(-c2ccccc2-c2sc(-c3cc4c(cc3-c3ccccc3)sc3ccccc34)c3ccccc23)cc1 ; c1ccc(-c2ccccc2-c2sc(-c3cc4c5ccccc5n(-c5ccccc5)c4cc3-c3ccccc3)c3ccccc23)cc1 ; c1ccc(-c2ccccc2-c2sc(-c3cc4oc5ccccc5c4cc3-c3ccccc3)c3ccccc23)cc1]

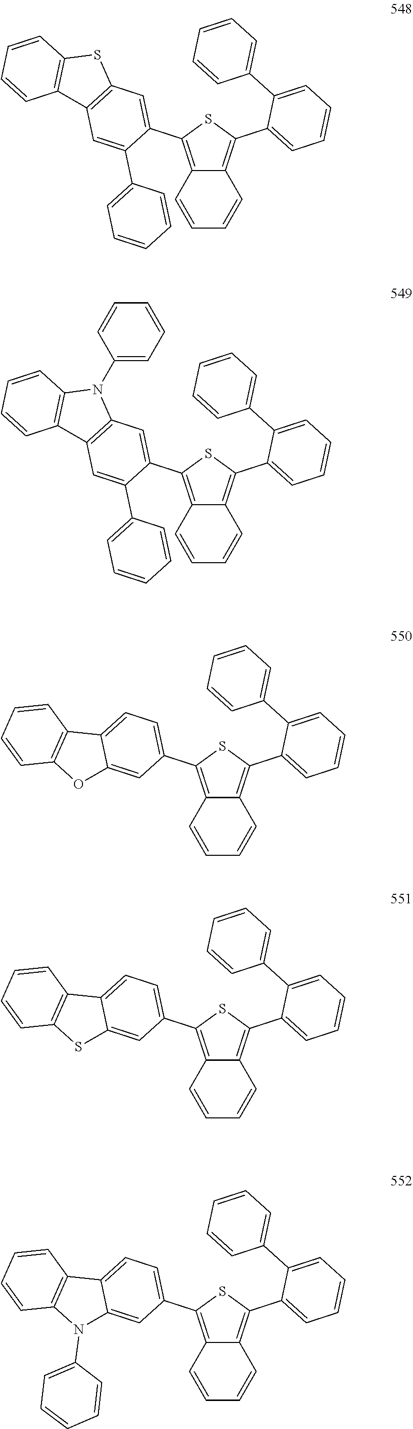 [SMILES: c1ccc(-c2ccccc2-c2sc(-c3cc4sc5ccccc5c4cc3-c3ccccc3)c3ccccc23)cc1 ; c1ccc(-c2ccccc2-c2sc(-c3cc4c(cc3-c3ccccc3)c3ccccc3n4-c3ccccc3)c3ccccc23)cc1 ; c1ccc(-c2ccccc2-c2sc(-c3ccc4c(c3)oc3ccccc34)c3ccccc23)cc1 ; c1ccc(-c2ccccc2-c2sc(-c3ccc4c(c3)sc3ccccc34)c3ccccc23)cc1 ; c1ccc(-c2ccccc2-c2sc(-c3ccc4c5ccccc5n(-c5ccccc5)c4c3)c3ccccc23)cc1]

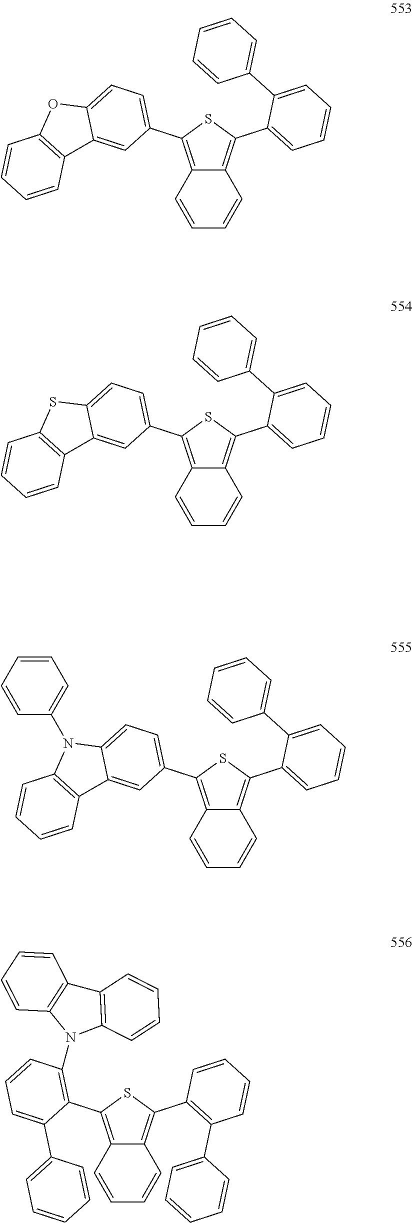 [SMILES: c1ccc(-c2ccccc2-c2sc(-c3ccc4oc5ccccc5c4c3)c3ccccc23)cc1 ; c1ccc(-c2ccccc2-c2sc(-c3ccc4sc5ccccc5c4c3)c3ccccc23)cc1 ; c1ccc(-c2ccccc2-c2sc(-c3ccc4c(c3)c3ccccc3n4-c3ccccc3)c3ccccc23)cc1 ; c1ccc(-c2ccccc2-c2sc(-c3c(-c4ccccc4)cccc3-n3c4ccccc4c4ccccc43)c3ccccc23)cc1]

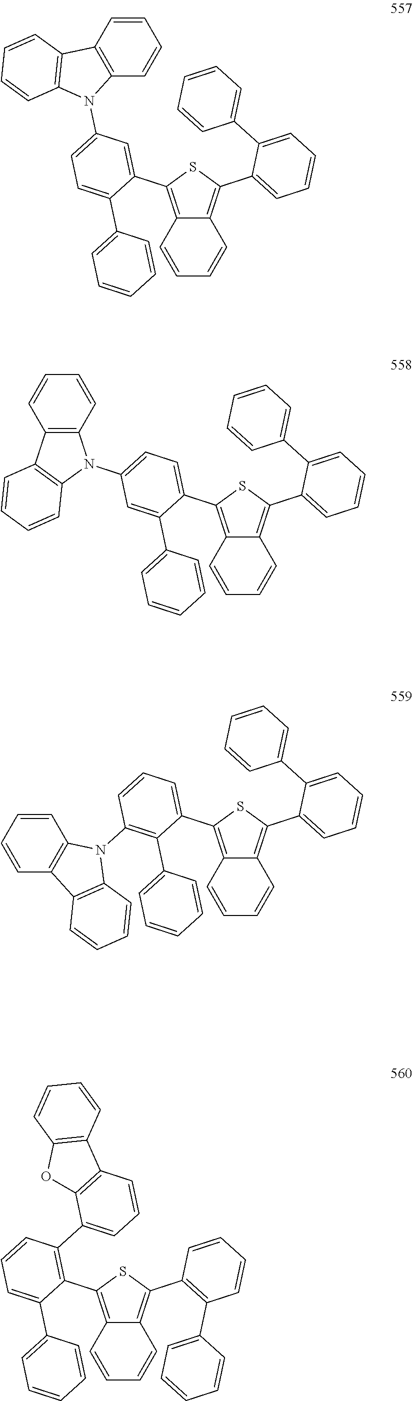 [SMILES: c1ccc(-c2ccccc2-c2sc(-c3cc(-n4c5ccccc5c5ccccc54)ccc3-c3ccccc3)c3ccccc23)cc1 ; c1ccc(-c2ccccc2-c2sc(-c3ccc(-n4c5ccccc5c5ccccc54)cc3-c3ccccc3)c3ccccc23)cc1 ; c1ccc(-c2ccccc2-c2sc(-c3cccc(-n4c5ccccc5c5ccccc54)c3-c3ccccc3)c3ccccc23)cc1 ; c1ccc(-c2ccccc2-c2sc(-c3c(-c4ccccc4)cccc3-c3cccc4c3oc3ccccc34)c3ccccc23)cc1]

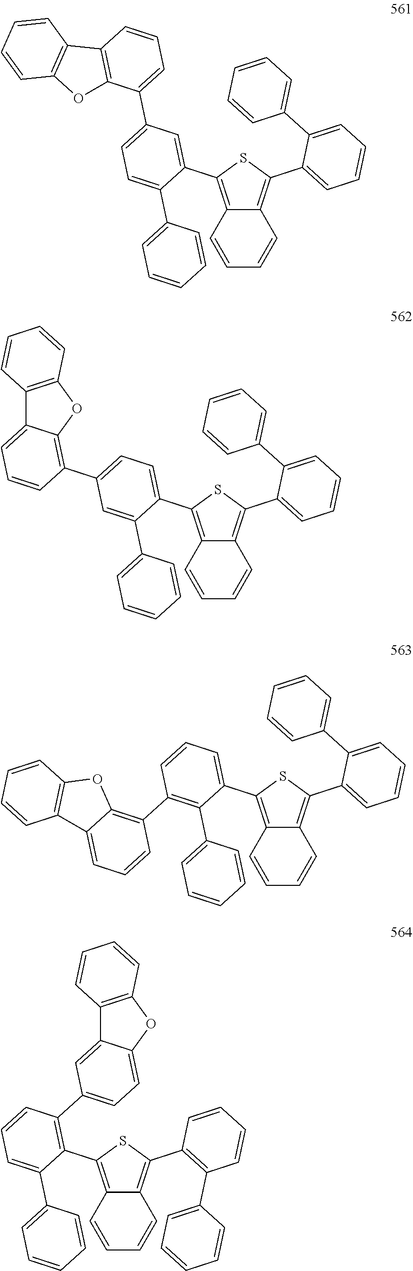 [SMILES: c1ccc(-c2ccccc2-c2sc(-c3cc(-c4cccc5c4oc4ccccc45)ccc3-c3ccccc3)c3ccccc23)cc1 ; c1ccc(-c2ccccc2-c2sc(-c3ccc(-c4cccc5c4oc4ccccc45)cc3-c3ccccc3)c3ccccc23)cc1 ; c1ccc(-c2ccccc2-c2sc(-c3cccc(-c4cccc5c4oc4ccccc45)c3-c3ccccc3)c3ccccc23)cc1 ; c1ccc(-c2ccccc2-c2sc(-c3c(-c4ccccc4)cccc3-c3ccc4oc5ccccc5c4c3)c3ccccc23)cc1]

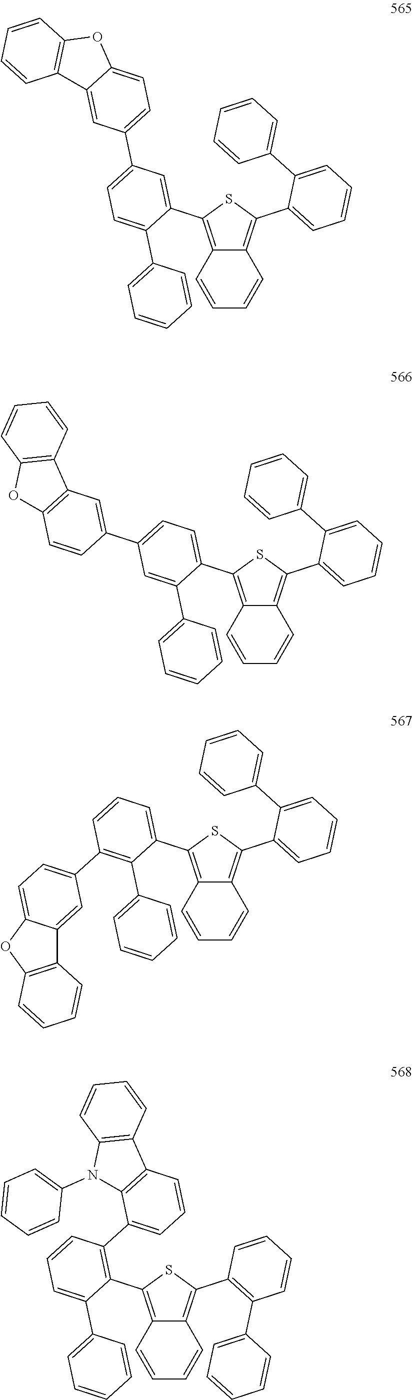 [SMILES: c1ccc(-c2ccccc2-c2sc(-c3cc(-c4ccc5oc6ccccc6c5c4)ccc3-c3ccccc3)c3ccccc23)cc1 ; c1ccc(-c2ccccc2-c2sc(-c3ccc(-c4ccc5oc6ccccc6c5c4)cc3-c3ccccc3)c3ccccc23)cc1 ; c1ccc(-c2ccccc2-c2sc(-c3cccc(-c4ccc5oc6ccccc6c5c4)c3-c3ccccc3)c3ccccc23)cc1 ; c1ccc(-c2ccccc2-c2sc(-c3c(-c4ccccc4)cccc3-c3cccc4c5ccccc5n(-c5ccccc5)c34)c3ccccc23)cc1]

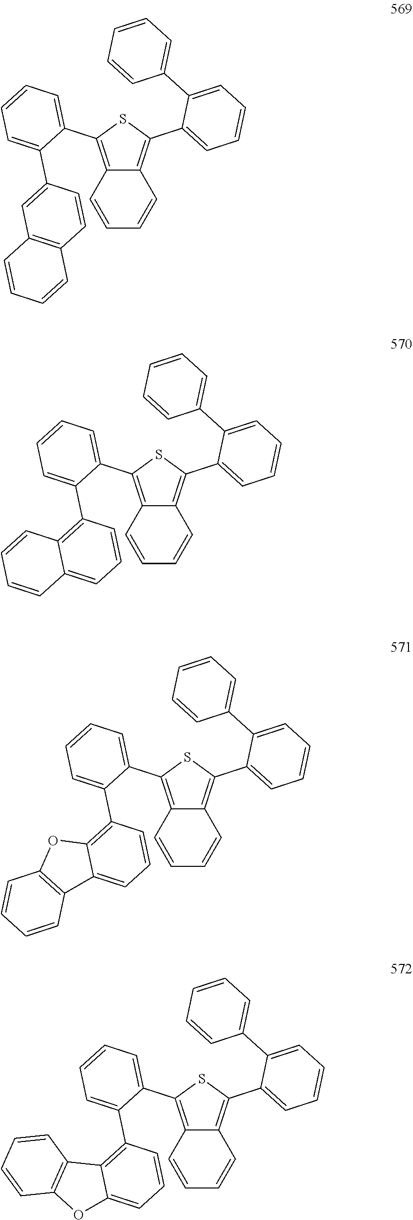 [SMILES: c1ccc(-c2ccccc2-c2sc(-c3ccccc3-c3ccc4ccccc4c3)c3ccccc23)cc1 ; c1ccc(-c2ccccc2-c2sc(-c3ccccc3-c3cccc4ccccc34)c3ccccc23)cc1 ; c1ccc(-c2ccccc2-c2sc(-c3ccccc3-c3cccc4c3oc3ccccc34)c3ccccc23)cc1 ; c1ccc(-c2ccccc2-c2sc(-c3ccccc3-c3cccc4oc5ccccc5c34)c3ccccc23)cc1]

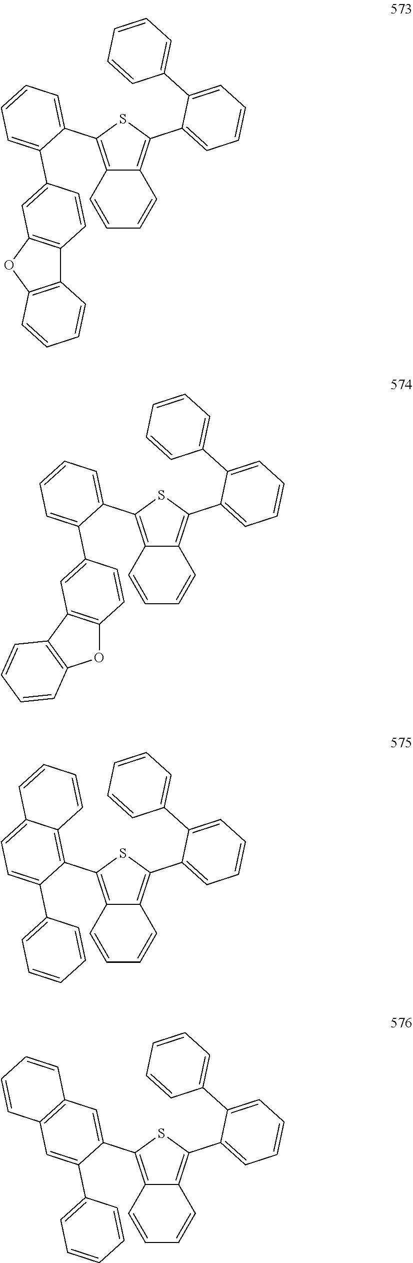 [SMILES: c1ccc(-c2ccccc2-c2sc(-c3ccccc3-c3ccc4c(c3)oc3ccccc34)c3ccccc23)cc1 ; c1ccc(-c2ccccc2-c2sc(-c3ccccc3-c3ccc4oc5ccccc5c4c3)c3ccccc23)cc1 ; c1ccc(-c2ccccc2-c2sc(-c3c(-c4ccccc4)ccc4ccccc34)c3ccccc23)cc1 ; c1ccc(-c2ccccc2-c2sc(-c3cc4ccccc4cc3-c3ccccc3)c3ccccc23)cc1]

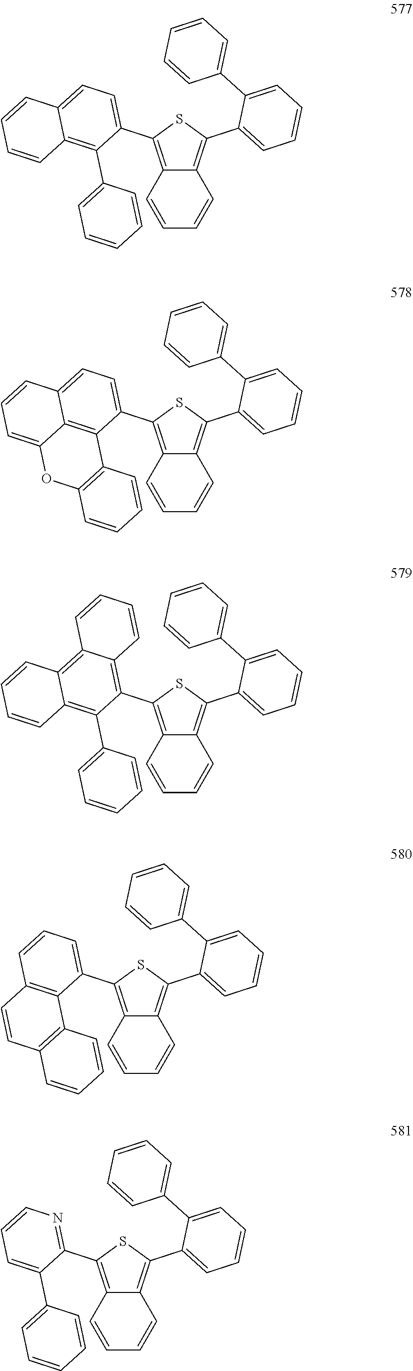 [SMILES: c1ccc(-c2ccccc2-c2sc(-c3ccc4ccccc4c3-c3ccccc3)c3ccccc23)cc1 ; c1ccc(-c2ccccc2-c2sc(-c3ccc4cccc5c4c3-c3ccccc3O5)c3ccccc23)cc1 ; c1ccc(-c2ccccc2-c2sc(-c3c(-c4ccccc4)c4ccccc4c4ccccc34)c3ccccc23)cc1 ; c1ccc(-c2ccccc2-c2sc(-c3cccc4ccc5ccccc5c34)c3ccccc23)cc1 ; c1ccc(-c2ccccc2-c2sc(-c3ncccc3-c3ccccc3)c3ccccc23)cc1]

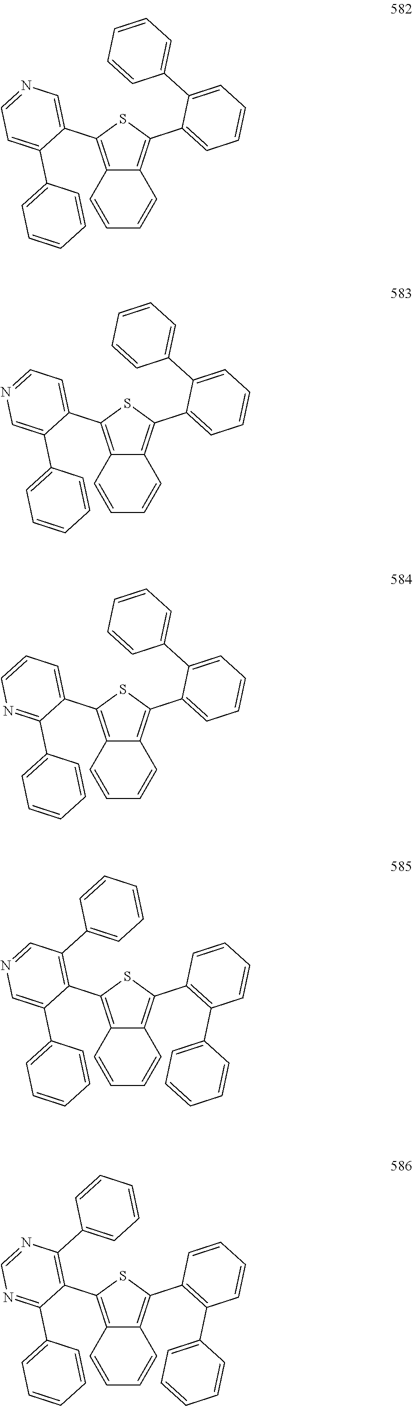 [SMILES: c1ccc(-c2ccccc2-c2sc(-c3cnccc3-c3ccccc3)c3ccccc23)cc1 ; c1ccc(-c2ccccc2-c2sc(-c3ccncc3-c3ccccc3)c3ccccc23)cc1 ; c1ccc(-c2ccccc2-c2sc(-c3cccnc3-c3ccccc3)c3ccccc23)cc1 ; c1ccc(-c2ccccc2-c2sc(-c3c(-c4ccccc4)cncc3-c3ccccc3)c3ccccc23)cc1 ; c1ccc(-c2ccccc2-c2sc(-c3c(-c4ccccc4)ncnc3-c3ccccc3)c3ccccc23)cc1]

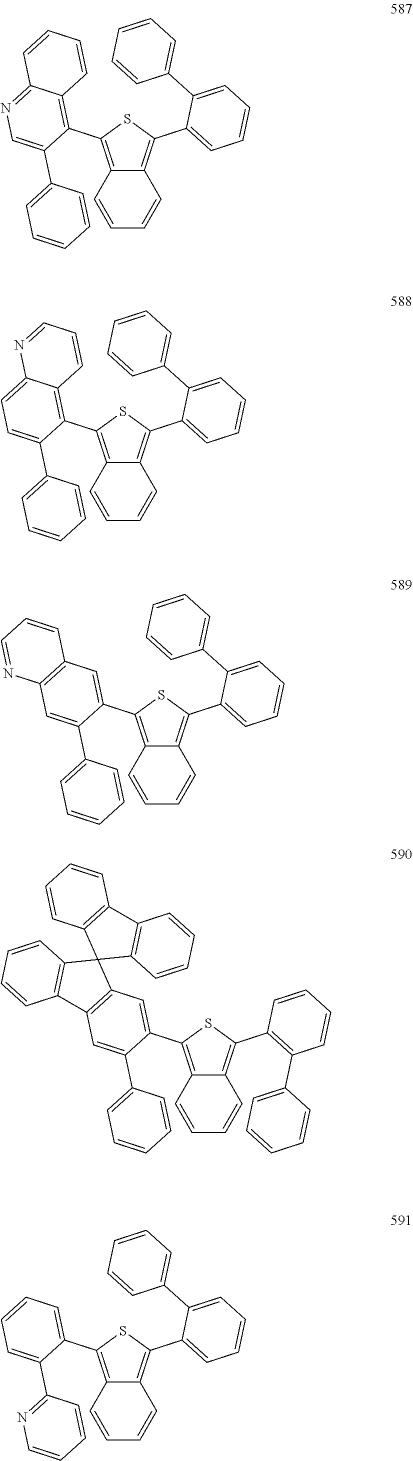 [SMILES: c1ccc(-c2ccccc2-c2sc(-c3c(-c4ccccc4)cnc4ccccc34)c3ccccc23)cc1 ; c1ccc(-c2ccccc2-c2sc(-c3c(-c4ccccc4)ccc4ncccc34)c3ccccc23)cc1 ; c1ccc(-c2ccccc2-c2sc(-c3cc4cccnc4cc3-c3ccccc3)c3ccccc23)cc1 ; c1ccc(-c2ccccc2-c2sc(-c3cc4c(cc3-c3ccccc3)-c3ccccc3C43c4ccccc4-c4ccccc43)c3ccccc23)cc1 ; c1ccc(-c2ccccc2-c2sc(-c3ccccc3-c3ccccn3)c3ccccc23)cc1]

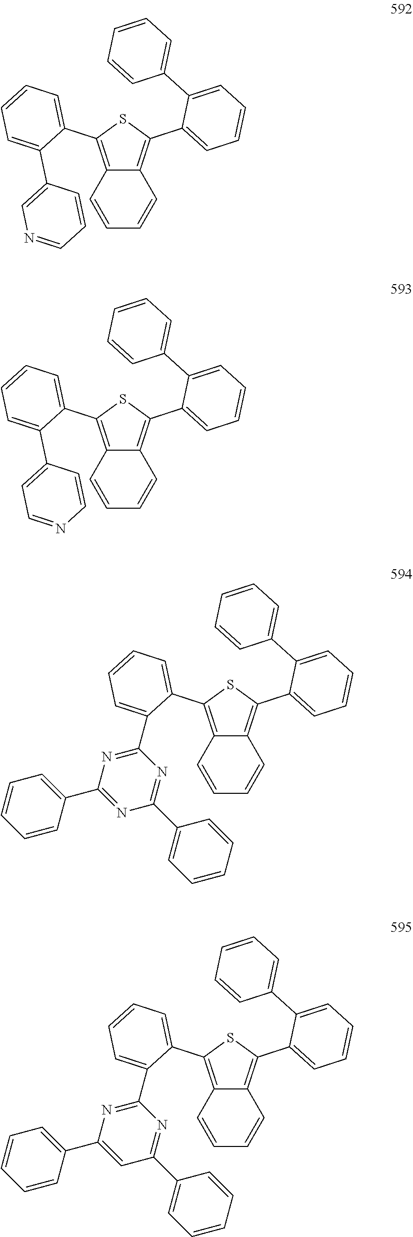 [SMILES: c1ccc(-c2ccccc2-c2sc(-c3ccccc3-c3cccnc3)c3ccccc23)cc1 ; c1ccc(-c2ccccc2-c2sc(-c3ccccc3-c3ccncc3)c3ccccc23)cc1 ; c1ccc(-c2nc(-c3ccccc3)nc(-c3ccccc3-c3sc(-c4ccccc4-c4ccccc4)c4ccccc34)n2)cc1 ; c1ccc(-c2cc(-c3ccccc3)nc(-c3ccccc3-c3sc(-c4ccccc4-c4ccccc4)c4ccccc34)n2)cc1]

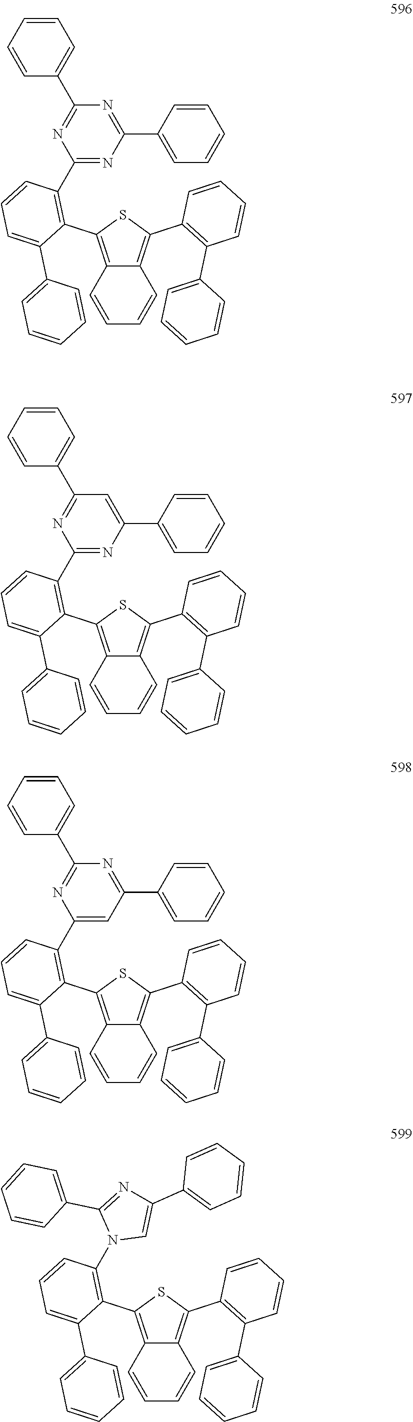

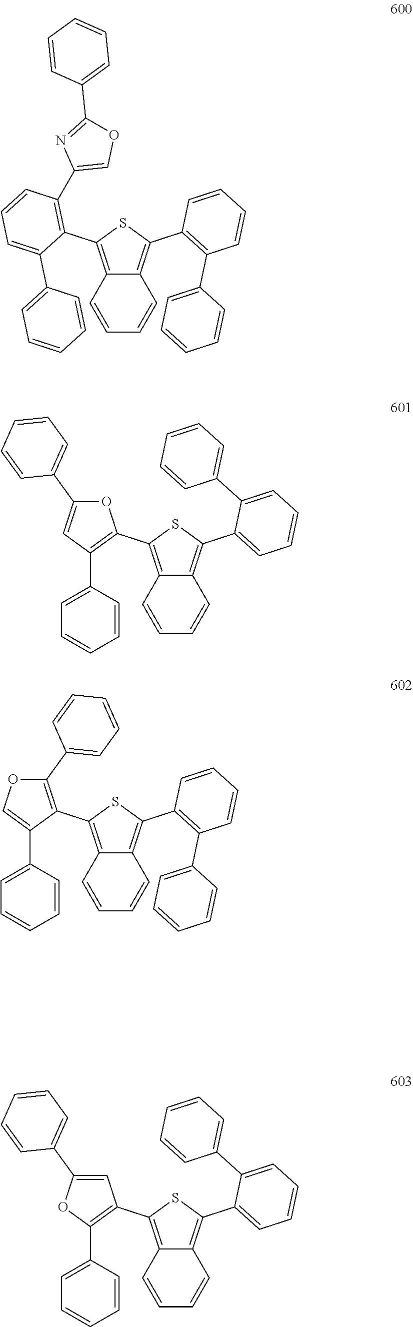

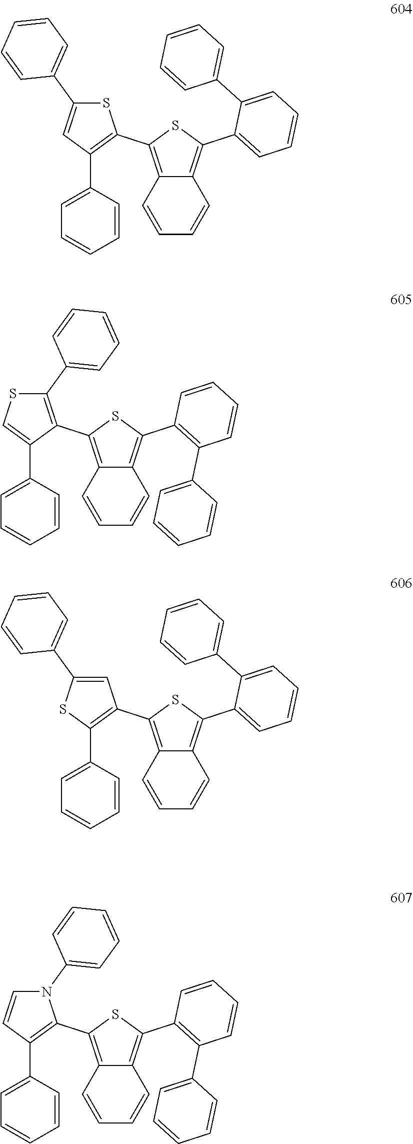 [SMILES: c1ccc(-c2cc(-c3ccccc3)c(-c3sc(-c4ccccc4-c4ccccc4)c4ccccc34)s2)cc1 ; c1ccc(-c2ccccc2-c2sc(-c3c(-c4ccccc4)csc3-c3ccccc3)c3ccccc23)cc1 ; c1ccc(-c2cc(-c3sc(-c4ccccc4-c4ccccc4)c4ccccc34)c(-c3ccccc3)s2)cc1 ; c1ccc(-c2ccccc2-c2sc(-c3c(-c4ccccc4)ccn3-c3ccccc3)c3ccccc23)cc1]

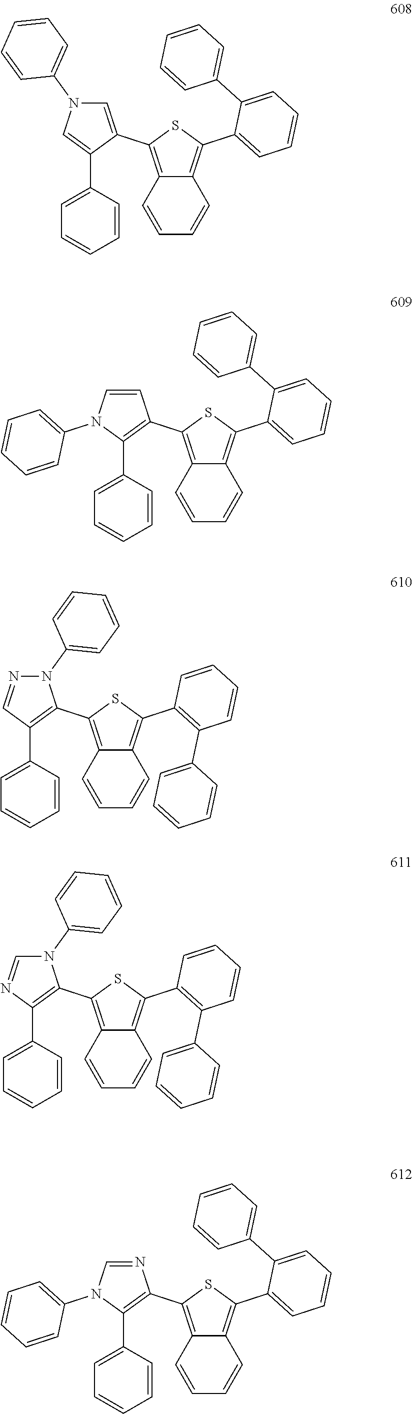 [SMILES: c1ccc(-c2ccccc2-c2sc(-c3cn(-c4ccccc4)cc3-c3ccccc3)c3ccccc23)cc1 ; c1ccc(-c2ccccc2-c2sc(-c3ccn(-c4ccccc4)c3-c3ccccc3)c3ccccc23)cc1 ; c1ccc(-c2ccccc2-c2sc(-c3c(-c4ccccc4)cnn3-c3ccccc3)c3ccccc23)cc1 ; c1ccc(-c2ccccc2-c2sc(-c3c(-c4ccccc4)ncn3-c3ccccc3)c3ccccc23)cc1 ; c1ccc(-c2ccccc2-c2sc(-c3ncn(-c4ccccc4)c3-c3ccccc3)c3ccccc23)cc1]

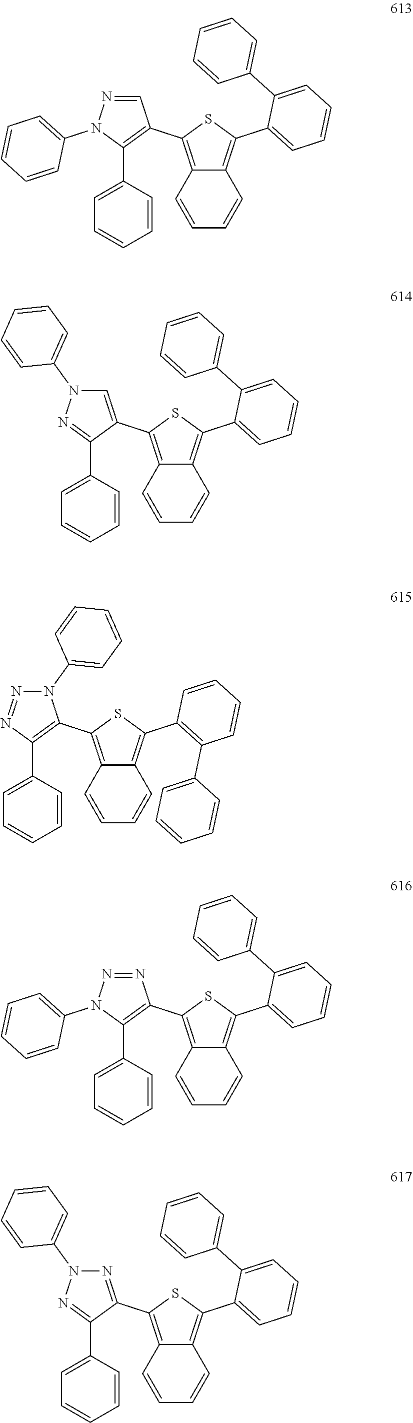 [SMILES: c1ccc(-c2ccccc2-c2sc(-c3cnn(-c4ccccc4)c3-c3ccccc3)c3ccccc23)cc1 ; c1ccc(-c2ccccc2-c2sc(-c3cn(-c4ccccc4)nc3-c3ccccc3)c3ccccc23)cc1 ; c1ccc(-c2ccccc2-c2sc(-c3c(-c4ccccc4)nnn3-c3ccccc3)c3ccccc23)cc1 ; c1ccc(-c2ccccc2-c2sc(-c3nnn(-c4ccccc4)c3-c3ccccc3)c3ccccc23)cc1 ; c1ccc(-c2ccccc2-c2sc(-c3nn(-c4ccccc4)nc3-c3ccccc3)c3ccccc23)cc1]

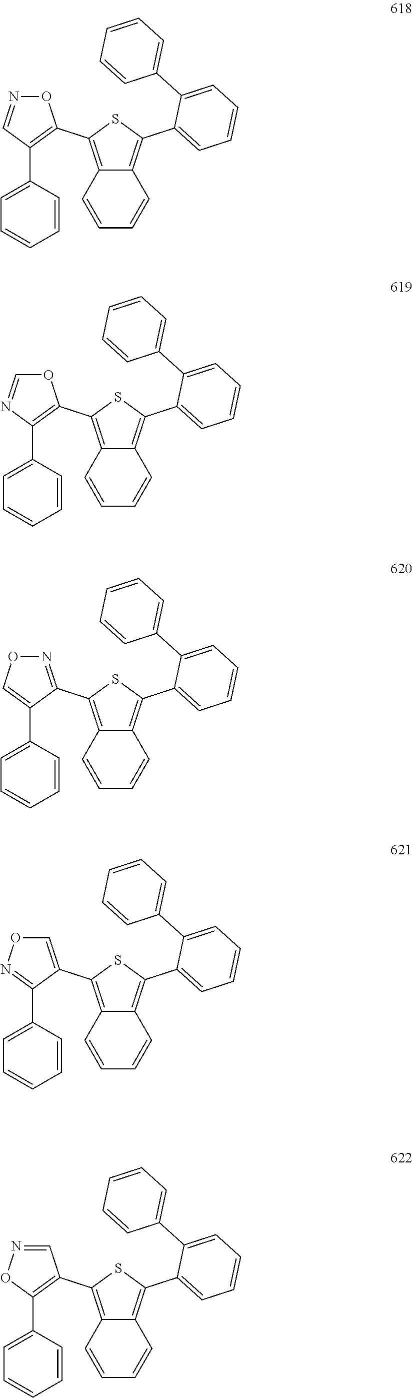 [SMILES: c1ccc(-c2ccccc2-c2sc(-c3oncc3-c3ccccc3)c3ccccc23)cc1 ; c1ccc(-c2ccccc2-c2sc(-c3ocnc3-c3ccccc3)c3ccccc23)cc1 ; c1ccc(-c2ccccc2-c2sc(-c3nocc3-c3ccccc3)c3ccccc23)cc1 ; c1ccc(-c2ccccc2-c2sc(-c3conc3-c3ccccc3)c3ccccc23)cc1 ; c1ccc(-c2ccccc2-c2sc(-c3cnoc3-c3ccccc3)c3ccccc23)cc1]

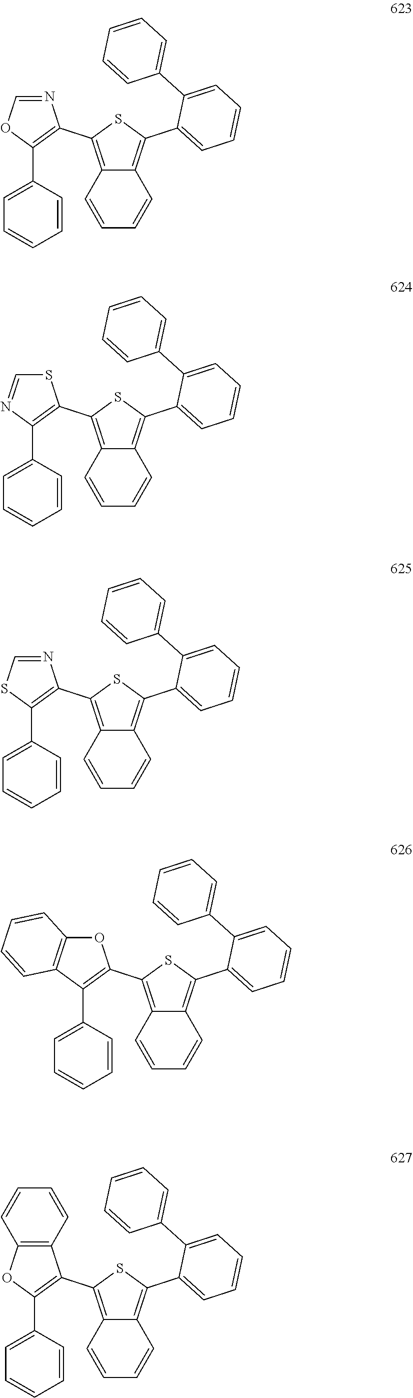 [SMILES: c1ccc(-c2ccccc2-c2sc(-c3ncoc3-c3ccccc3)c3ccccc23)cc1 ; c1ccc(-c2ccccc2-c2sc(-c3scnc3-c3ccccc3)c3ccccc23)cc1 ; c1ccc(-c2ccccc2-c2sc(-c3ncsc3-c3ccccc3)c3ccccc23)cc1 ; c1ccc(-c2ccccc2-c2sc(-c3oc4ccccc4c3-c3ccccc3)c3ccccc23)cc1 ; c1ccc(-c2ccccc2-c2sc(-c3c(-c4ccccc4)oc4ccccc34)c3ccccc23)cc1]

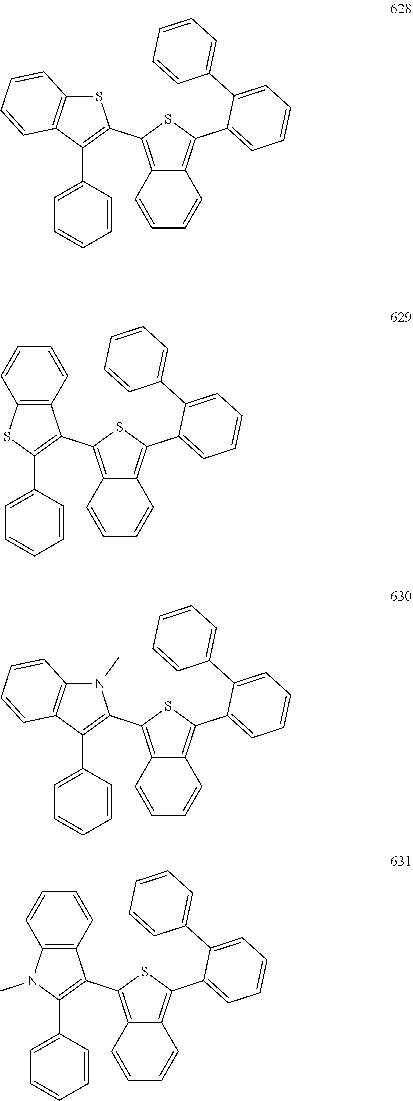 [SMILES: c1ccc(-c2ccccc2-c2sc(-c3sc4ccccc4c3-c3ccccc3)c3ccccc23)cc1 ; c1ccc(-c2ccccc2-c2sc(-c3c(-c4ccccc4)sc4ccccc34)c3ccccc23)cc1 ; Cn1c(-c2sc(-c3ccccc3-c3ccccc3)c3ccccc23)c(-c2ccccc2)c2ccccc21 ; Cn1c(-c2ccccc2)c(-c2sc(-c3ccccc3-c3ccccc3)c3ccccc23)c2ccccc21]

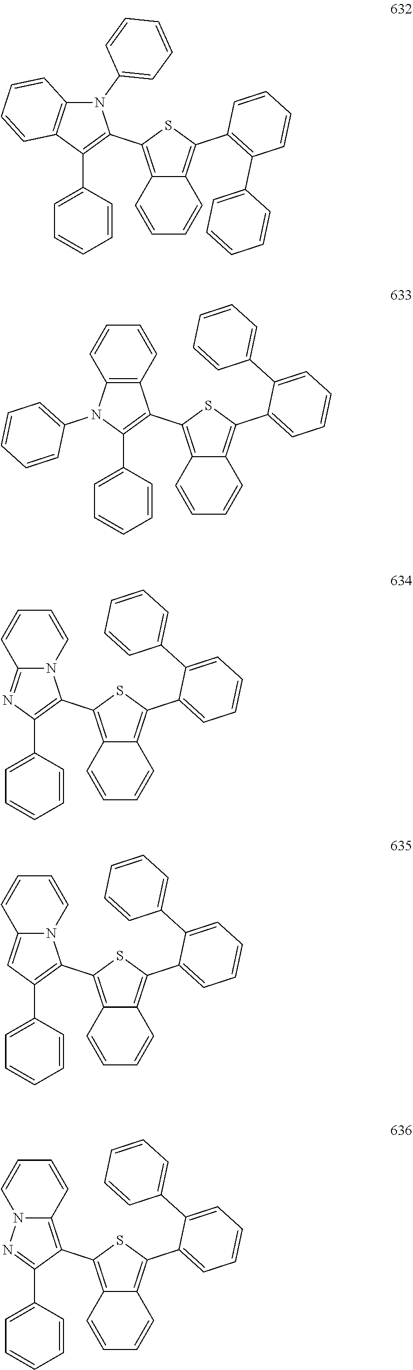 [SMILES: c1ccc(-c2ccccc2-c2sc(-c3c(-c4ccccc4)c4ccccc4n3-c3ccccc3)c3ccccc23)cc1 ; c1ccc(-c2ccccc2-c2sc(-c3c(-c4ccccc4)n(-c4ccccc4)c4ccccc34)c3ccccc23)cc1 ; c1ccc(-c2ccccc2-c2sc(-c3c(-c4ccccc4)nc4ccccn34)c3ccccc23)cc1 ; c1ccc(-c2ccccc2-c2sc(-c3c(-c4ccccc4)cc4ccccn34)c3ccccc23)cc1 ; c1ccc(-c2ccccc2-c2sc(-c3c(-c4ccccc4)nn4ccccc34)c3ccccc23)cc1]

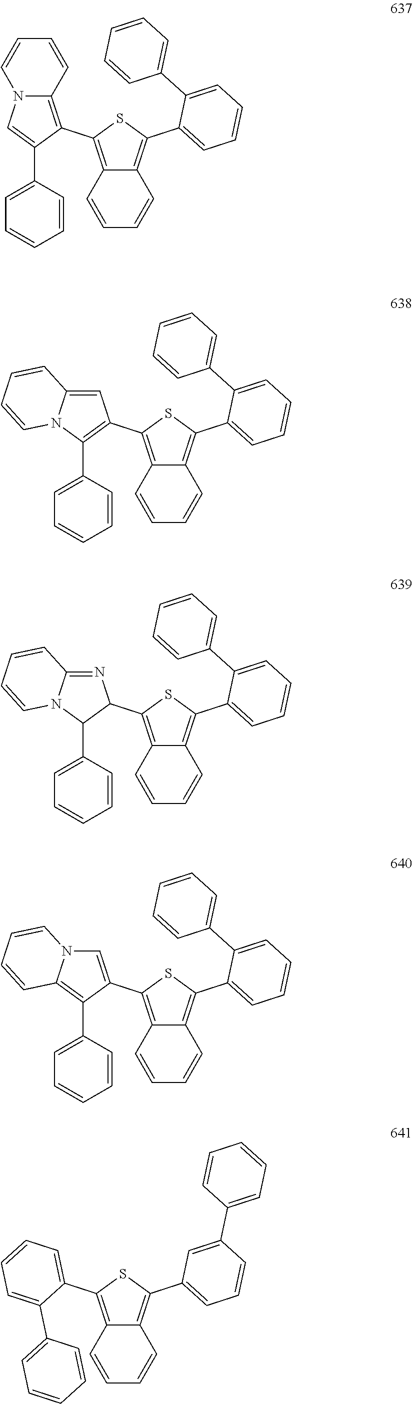 [SMILES: c1ccc(-c2ccccc2-c2sc(-c3c(-c4ccccc4)cn4ccccc34)c3ccccc23)cc1 ; c1ccc(-c2ccccc2-c2sc(-c3cc4ccccn4c3-c3ccccc3)c3ccccc23)cc1 ; C1=CC2=NC(c3sc(-c4ccccc4-c4ccccc4)c4ccccc34)C(c3ccccc3)N2C=C1 ; c1ccc(-c2ccccc2-c2sc(-c3cn4ccccc4c3-c3ccccc3)c3ccccc23)cc1 ; c1ccc(-c2cccc(-c3sc(-c4ccccc4-c4ccccc4)c4ccccc34)c2)cc1]

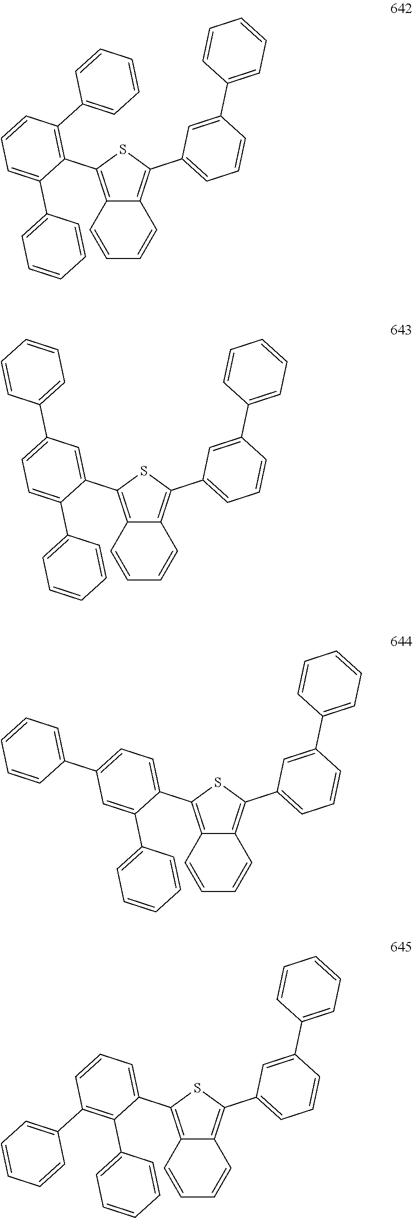 [SMILES: c1ccc(-c2cccc(-c3sc(-c4c(-c5ccccc5)cccc4-c4ccccc4)c4ccccc34)c2)cc1 ; c1ccc(-c2cccc(-c3sc(-c4cc(-c5ccccc5)ccc4-c4ccccc4)c4ccccc34)c2)cc1 ; c1ccc(-c2cccc(-c3sc(-c4ccc(-c5ccccc5)cc4-c4ccccc4)c4ccccc34)c2)cc1 ; c1ccc(-c2cccc(-c3sc(-c4cccc(-c5ccccc5)c4-c4ccccc4)c4ccccc34)c2)cc1]

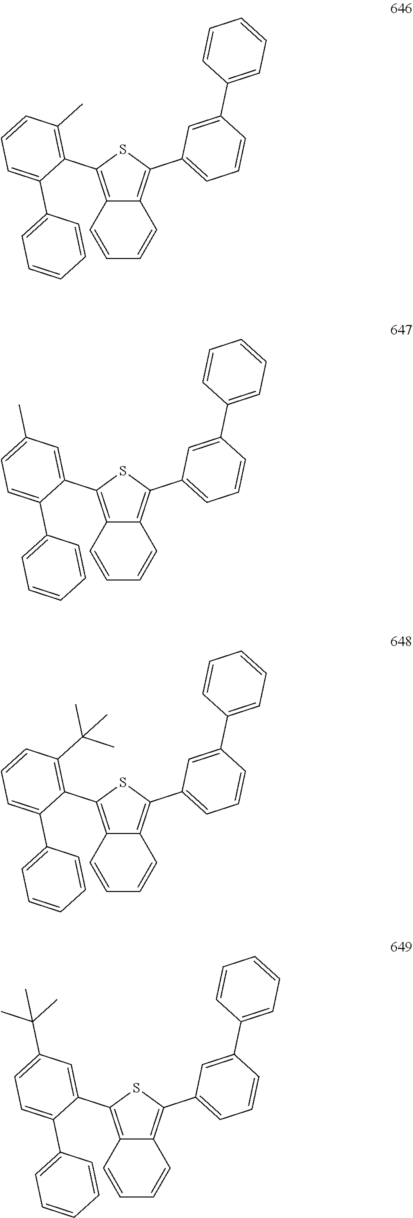 [SMILES: Cc1cccc(-c2ccccc2)c1-c1sc(-c2cccc(-c3ccccc3)c2)c2ccccc12 ; Cc1ccc(-c2ccccc2)c(-c2sc(-c3cccc(-c4ccccc4)c3)c3ccccc23)c1 ; CC(C)(C)c1cccc(-c2ccccc2)c1-c1sc(-c2cccc(-c3ccccc3)c2)c2ccccc12 ; CC(C)(C)c1ccc(-c2ccccc2)c(-c2sc(-c3cccc(-c4ccccc4)c3)c3ccccc23)c1]

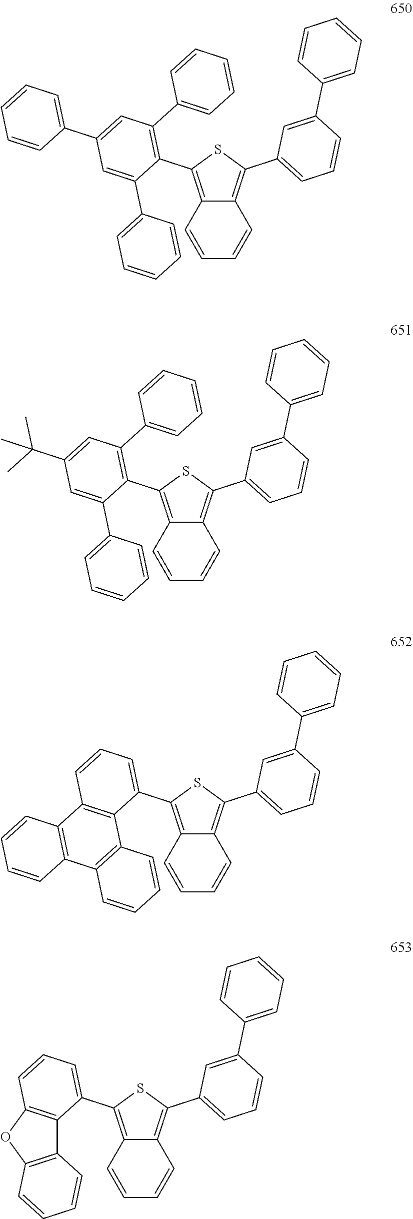 [SMILES: c1ccc(-c2cccc(-c3sc(-c4c(-c5ccccc5)cc(-c5ccccc5)cc4-c4ccccc4)c4ccccc34)c2)cc1 ; CC(C)(C)c1cc(-c2ccccc2)c(-c2sc(-c3cccc(-c4ccccc4)c3)c3ccccc23)c(-c2ccccc2)c1 ; c1ccc(-c2cccc(-c3sc(-c4cccc5c6ccccc6c6ccccc6c45)c4ccccc34)c2)cc1 ; c1ccc(-c2cccc(-c3sc(-c4cccc5oc6ccccc6c45)c4ccccc34)c2)cc1]

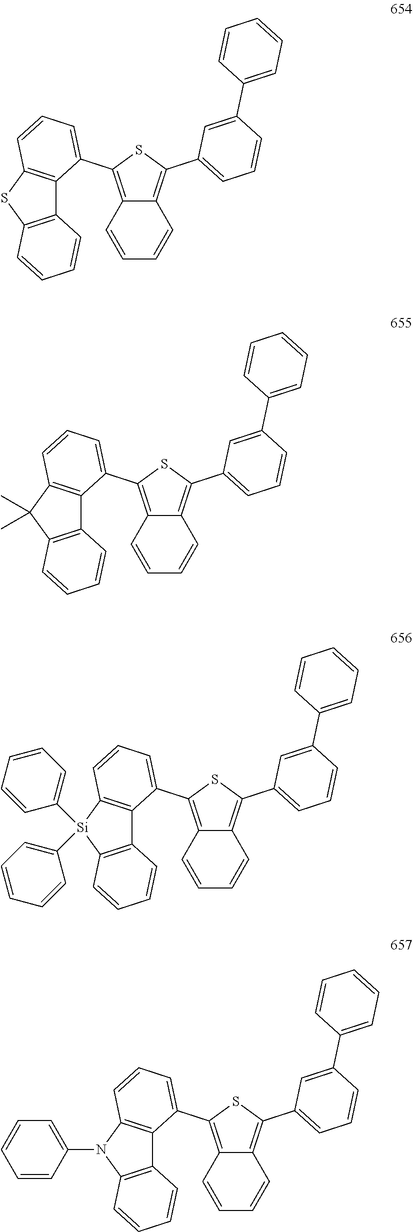 [SMILES: c1ccc(-c2cccc(-c3sc(-c4cccc5sc6ccccc6c45)c4ccccc34)c2)cc1 ; CC1(C)c2ccccc2-c2c(-c3sc(-c4cccc(-c5ccccc5)c4)c4ccccc34)cccc21 ; c1ccc(-c2cccc(-c3sc(-c4cccc5c4-c4ccccc4[Si]5(c4ccccc4)c4ccccc4)c4ccccc34)c2)cc1 ; c1ccc(-c2cccc(-c3sc(-c4cccc5c4c4ccccc4n5-c4ccccc4)c4ccccc34)c2)cc1]

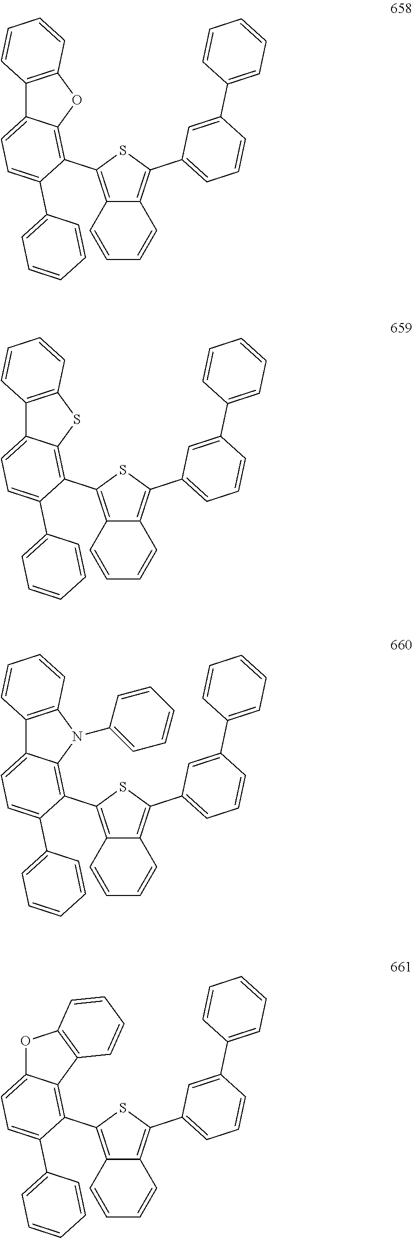 [SMILES: c1ccc(-c2cccc(-c3sc(-c4c(-c5ccccc5)ccc5c4oc4ccccc45)c4ccccc34)c2)cc1 ; c1ccc(-c2cccc(-c3sc(-c4c(-c5ccccc5)ccc5c4sc4ccccc45)c4ccccc34)c2)cc1 ; c1ccc(-c2cccc(-c3sc(-c4c(-c5ccccc5)ccc5c6ccccc6n(-c6ccccc6)c45)c4ccccc34)c2)cc1 ; c1ccc(-c2cccc(-c3sc(-c4c(-c5ccccc5)ccc5oc6ccccc6c45)c4ccccc34)c2)cc1]

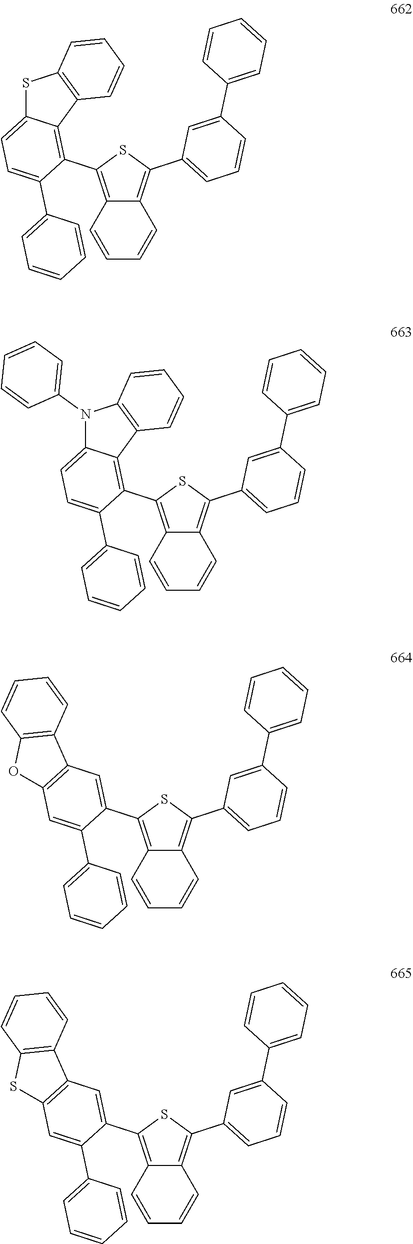 [SMILES: c1ccc(-c2cccc(-c3sc(-c4c(-c5ccccc5)ccc5sc6ccccc6c45)c4ccccc34)c2)cc1 ; c1ccc(-c2cccc(-c3sc(-c4c(-c5ccccc5)ccc5c4c4ccccc4n5-c4ccccc4)c4ccccc34)c2)cc1 ; c1ccc(-c2cccc(-c3sc(-c4cc5c(cc4-c4ccccc4)oc4ccccc45)c4ccccc34)c2)cc1 ; c1ccc(-c2cccc(-c3sc(-c4cc5c(cc4-c4ccccc4)sc4ccccc45)c4ccccc34)c2)cc1]

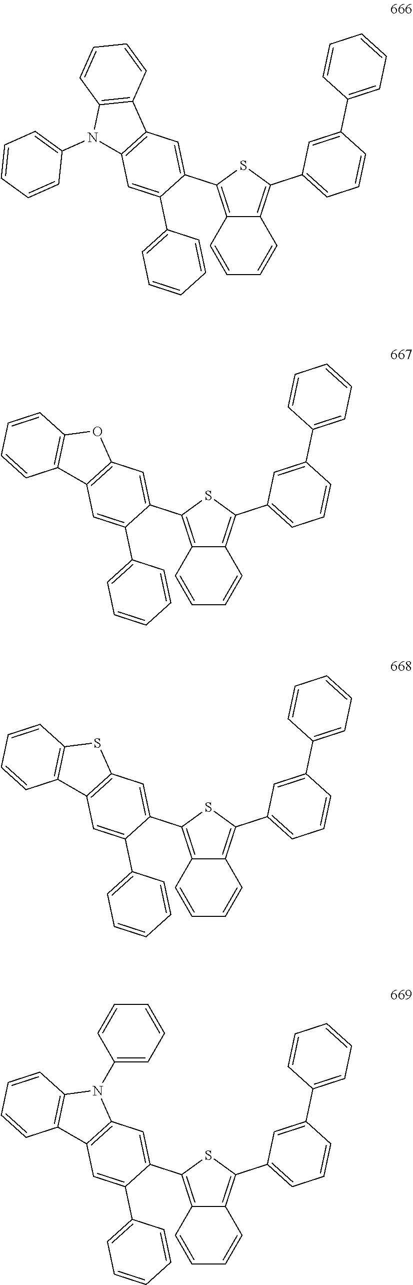 [SMILES: c1ccc(-c2cccc(-c3sc(-c4cc5c6ccccc6n(-c6ccccc6)c5cc4-c4ccccc4)c4ccccc34)c2)cc1 ; c1ccc(-c2cccc(-c3sc(-c4cc5oc6ccccc6c5cc4-c4ccccc4)c4ccccc34)c2)cc1 ; c1ccc(-c2cccc(-c3sc(-c4cc5sc6ccccc6c5cc4-c4ccccc4)c4ccccc34)c2)cc1 ; c1ccc(-c2cccc(-c3sc(-c4cc5c(cc4-c4ccccc4)c4ccccc4n5-c4ccccc4)c4ccccc34)c2)cc1]

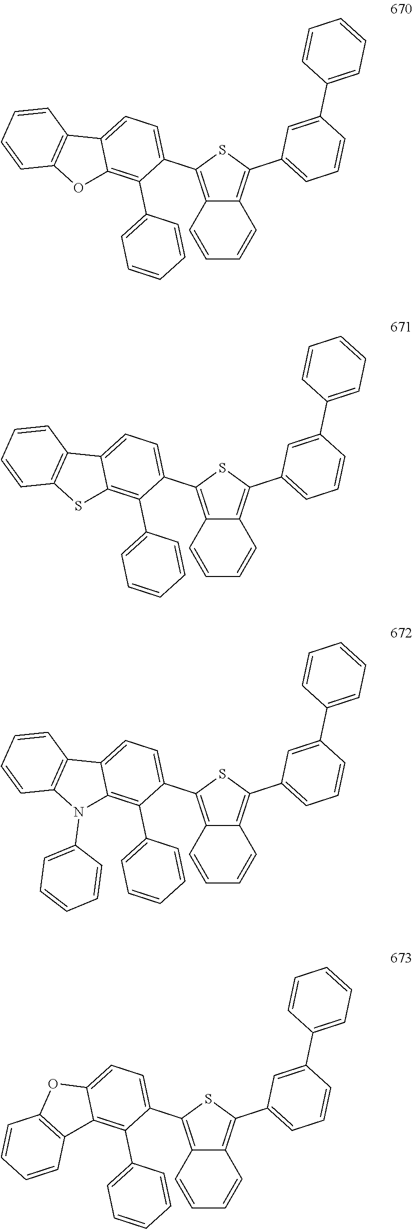 [SMILES: c1ccc(-c2cccc(-c3sc(-c4ccc5c(oc6ccccc65)c4-c4ccccc4)c4ccccc34)c2)cc1 ; c1ccc(-c2cccc(-c3sc(-c4ccc5c(sc6ccccc65)c4-c4ccccc4)c4ccccc34)c2)cc1 ; c1ccc(-c2cccc(-c3sc(-c4ccc5c6ccccc6n(-c6ccccc6)c5c4-c4ccccc4)c4ccccc34)c2)cc1 ; c1ccc(-c2cccc(-c3sc(-c4ccc5oc6ccccc6c5c4-c4ccccc4)c4ccccc34)c2)cc1]

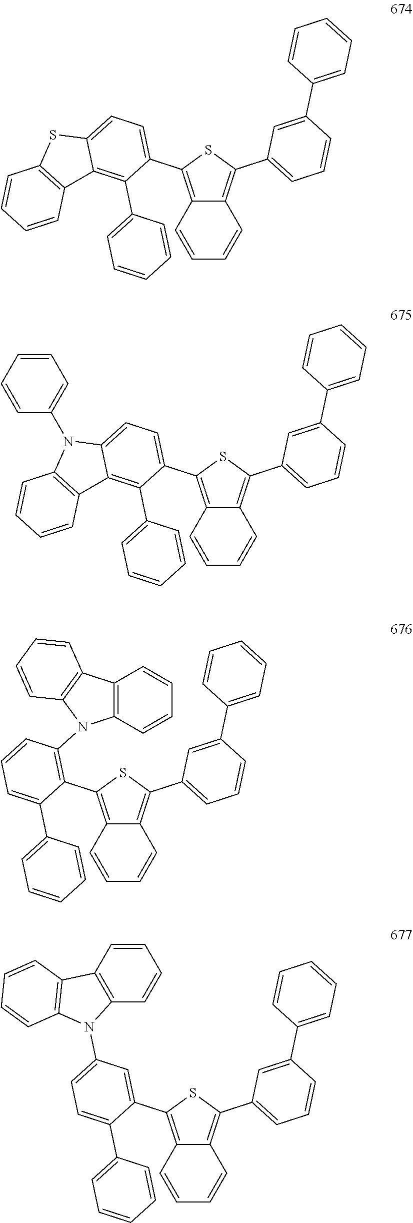 [SMILES: c1ccc(-c2cccc(-c3sc(-c4ccc5sc6ccccc6c5c4-c4ccccc4)c4ccccc34)c2)cc1 ; c1ccc(-c2cccc(-c3sc(-c4ccc5c(c4-c4ccccc4)c4ccccc4n5-c4ccccc4)c4ccccc34)c2)cc1 ; c1ccc(-c2cccc(-c3sc(-c4c(-c5ccccc5)cccc4-n4c5ccccc5c5ccccc54)c4ccccc34)c2)cc1 ; c1ccc(-c2cccc(-c3sc(-c4cc(-n5c6ccccc6c6ccccc65)ccc4-c4ccccc4)c4ccccc34)c2)cc1]

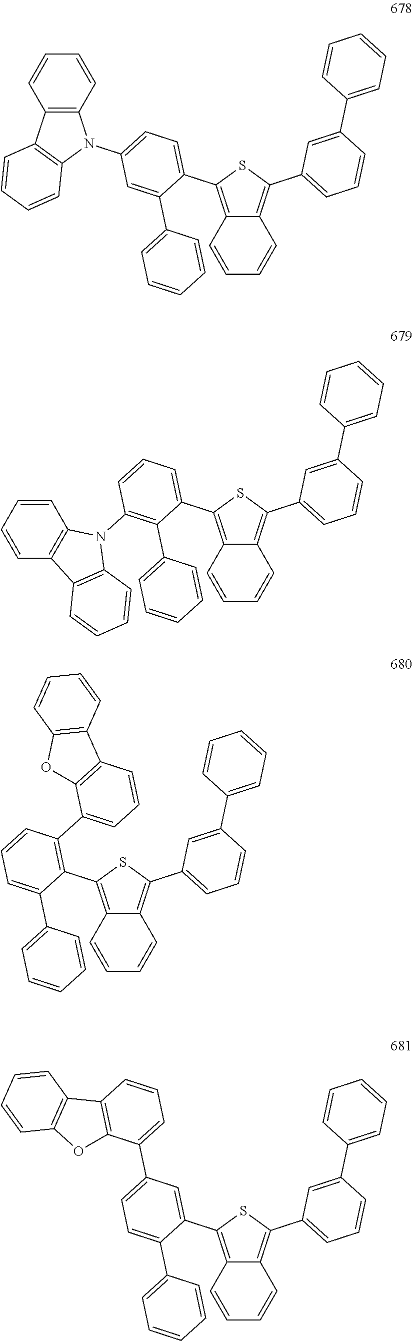

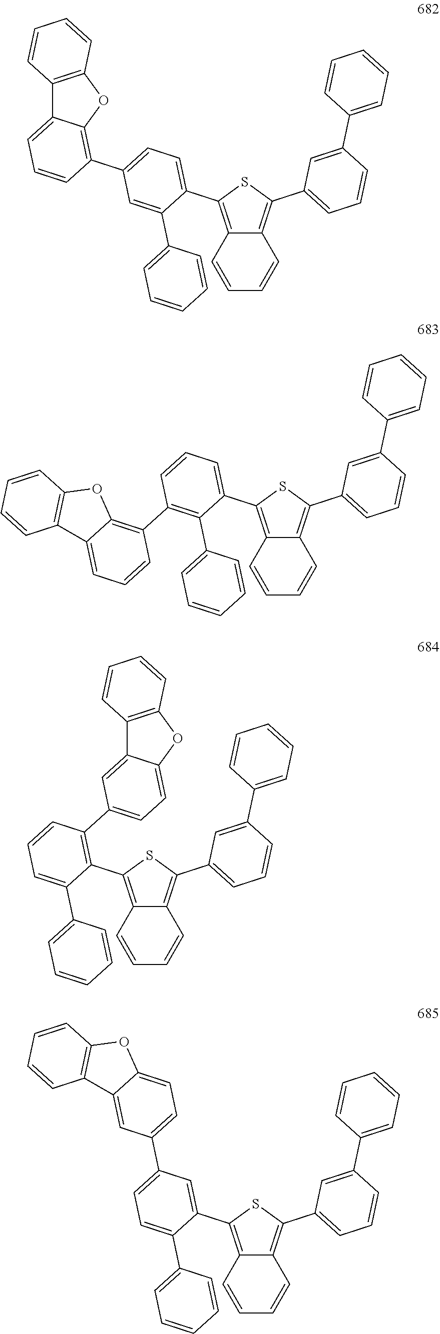

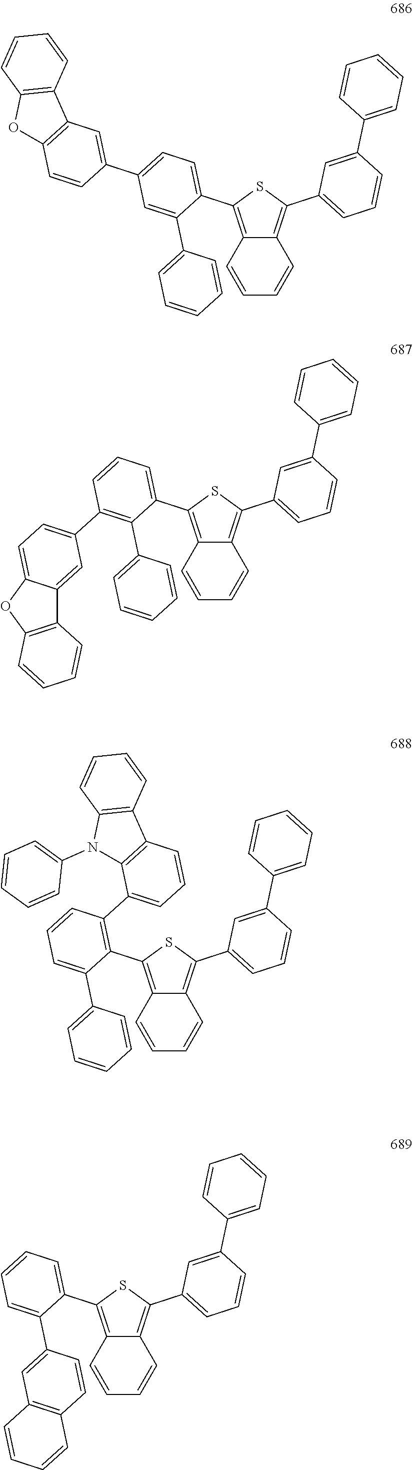 [SMILES: c1ccc(-c2cccc(-c3sc(-c4ccc(-c5ccc6oc7ccccc7c6c5)cc4-c4ccccc4)c4ccccc34)c2)cc1 ; c1ccc(-c2cccc(-c3sc(-c4cccc(-c5ccc6oc7ccccc7c6c5)c4-c4ccccc4)c4ccccc34)c2)cc1 ; c1ccc(-c2cccc(-c3sc(-c4c(-c5ccccc5)cccc4-c4cccc5c6ccccc6n(-c6ccccc6)c45)c4ccccc34)c2)cc1 ; c1ccc(-c2cccc(-c3sc(-c4ccccc4-c4ccc5ccccc5c4)c4ccccc34)c2)cc1]

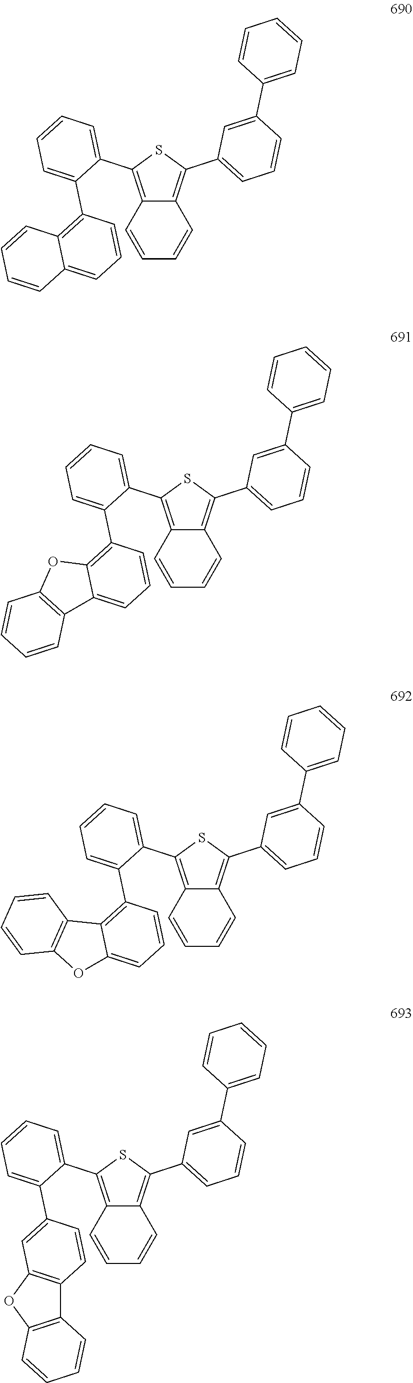 [SMILES: c1ccc(-c2cccc(-c3sc(-c4ccccc4-c4cccc5ccccc45)c4ccccc34)c2)cc1 ; c1ccc(-c2cccc(-c3sc(-c4ccccc4-c4cccc5c4oc4ccccc45)c4ccccc34)c2)cc1 ; c1ccc(-c2cccc(-c3sc(-c4ccccc4-c4cccc5oc6ccccc6c45)c4ccccc34)c2)cc1 ; c1ccc(-c2cccc(-c3sc(-c4ccccc4-c4ccc5c(c4)oc4ccccc45)c4ccccc34)c2)cc1]

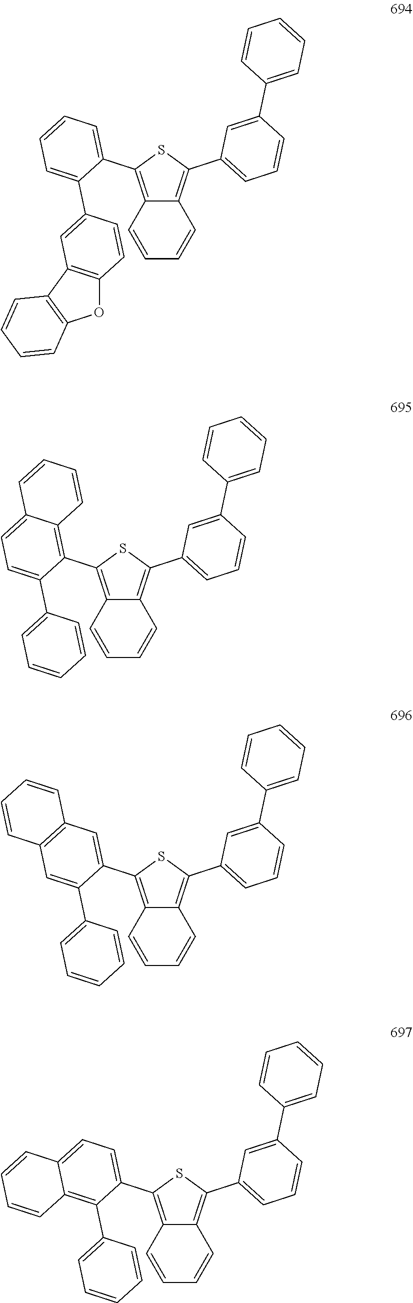 [SMILES: c1ccc(-c2cccc(-c3sc(-c4ccccc4-c4ccc5oc6ccccc6c5c4)c4ccccc34)c2)cc1 ; c1ccc(-c2cccc(-c3sc(-c4c(-c5ccccc5)ccc5ccccc45)c4ccccc34)c2)cc1 ; c1ccc(-c2cccc(-c3sc(-c4cc5ccccc5cc4-c4ccccc4)c4ccccc34)c2)cc1 ; c1ccc(-c2cccc(-c3sc(-c4ccc5ccccc5c4-c4ccccc4)c4ccccc34)c2)cc1]

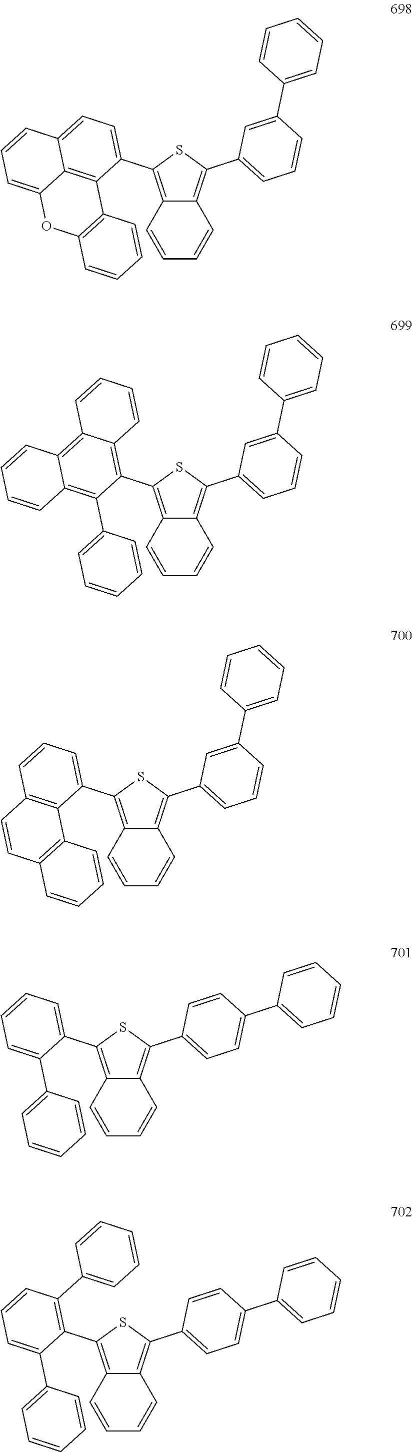 [SMILES: c1ccc(-c2cccc(-c3sc(-c4ccc5cccc6c5c4-c4ccccc4O6)c4ccccc34)c2)cc1 ; c1ccc(-c2cccc(-c3sc(-c4c(-c5ccccc5)c5ccccc5c5ccccc45)c4ccccc34)c2)cc1 ; c1ccc(-c2cccc(-c3sc(-c4cccc5ccc6ccccc6c45)c4ccccc34)c2)cc1 ; c1ccc(-c2ccc(-c3sc(-c4ccccc4-c4ccccc4)c4ccccc34)cc2)cc1 ; c1ccc(-c2ccc(-c3sc(-c4c(-c5ccccc5)cccc4-c4ccccc4)c4ccccc34)cc2)cc1]

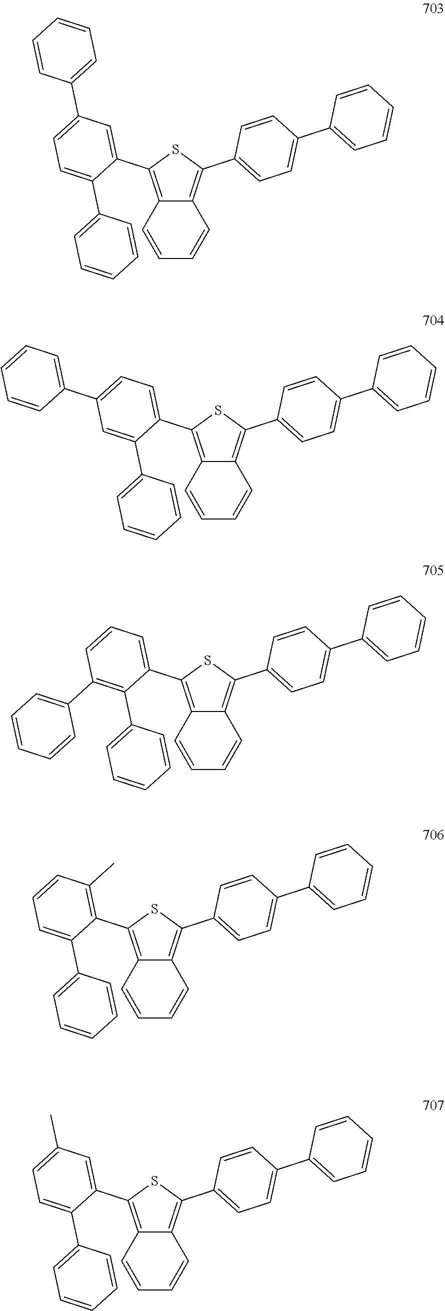 [SMILES: c1ccc(-c2ccc(-c3sc(-c4cc(-c5ccccc5)ccc4-c4ccccc4)c4ccccc34)cc2)cc1 ; c1ccc(-c2ccc(-c3sc(-c4ccc(-c5ccccc5)cc4-c4ccccc4)c4ccccc34)cc2)cc1 ; c1ccc(-c2ccc(-c3sc(-c4cccc(-c5ccccc5)c4-c4ccccc4)c4ccccc34)cc2)cc1 ; Cc1cccc(-c2ccccc2)c1-c1sc(-c2ccc(-c3ccccc3)cc2)c2ccccc12 ; Cc1ccc(-c2ccccc2)c(-c2sc(-c3ccc(-c4ccccc4)cc3)c3ccccc23)c1]

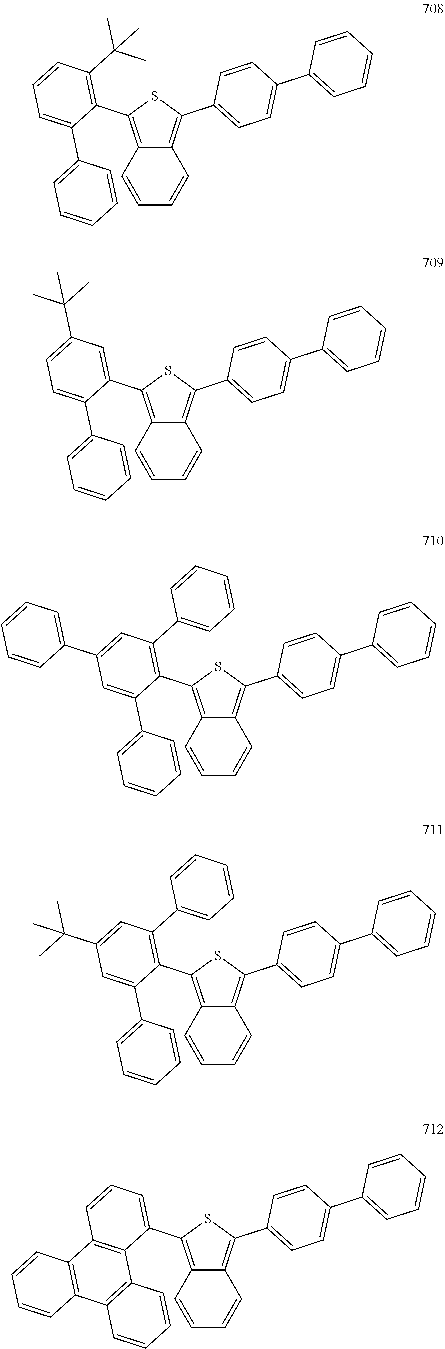 [SMILES: CC(C)(C)c1cccc(-c2ccccc2)c1-c1sc(-c2ccc(-c3ccccc3)cc2)c2ccccc12 ; CC(C)(C)c1ccc(-c2ccccc2)c(-c2sc(-c3ccc(-c4ccccc4)cc3)c3ccccc23)c1 ; c1ccc(-c2ccc(-c3sc(-c4c(-c5ccccc5)cc(-c5ccccc5)cc4-c4ccccc4)c4ccccc34)cc2)cc1 ; CC(C)(C)c1cc(-c2ccccc2)c(-c2sc(-c3ccc(-c4ccccc4)cc3)c3ccccc23)c(-c2ccccc2)c1 ; c1ccc(-c2ccc(-c3sc(-c4cccc5c6ccccc6c6ccccc6c45)c4ccccc34)cc2)cc1]

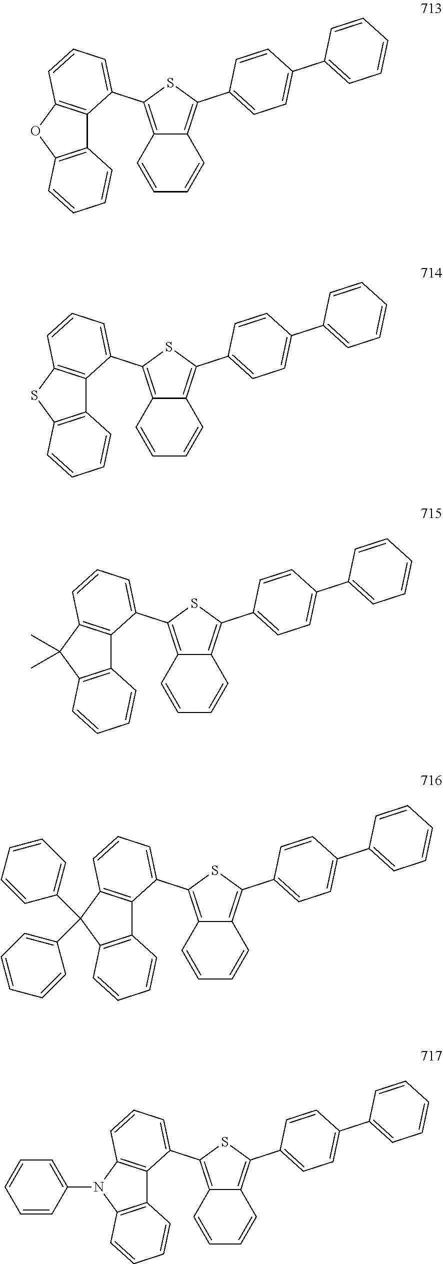 [SMILES: c1ccc(-c2ccc(-c3sc(-c4cccc5oc6ccccc6c45)c4ccccc34)cc2)cc1 ; c1ccc(-c2ccc(-c3sc(-c4cccc5sc6ccccc6c45)c4ccccc34)cc2)cc1 ; CC1(C)c2ccccc2-c2c(-c3sc(-c4ccc(-c5ccccc5)cc4)c4ccccc34)cccc21 ; c1ccc(-c2ccc(-c3sc(-c4cccc5c4-c4ccccc4C5(c4ccccc4)c4ccccc4)c4ccccc34)cc2)cc1 ; c1ccc(-c2ccc(-c3sc(-c4cccc5c4c4ccccc4n5-c4ccccc4)c4ccccc34)cc2)cc1]

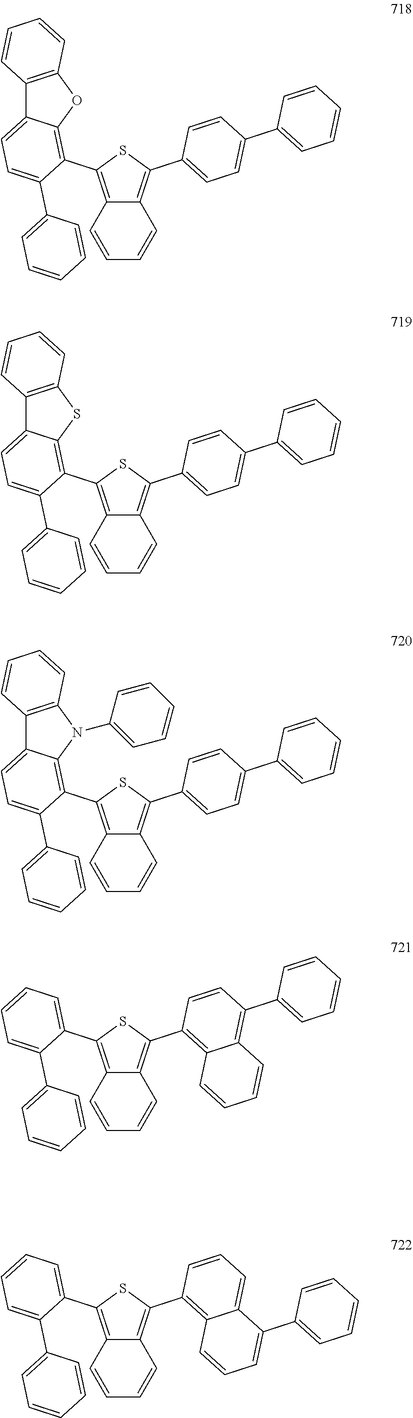 [SMILES: c1ccc(-c2ccc(-c3sc(-c4c(-c5ccccc5)ccc5c4oc4ccccc45)c4ccccc34)cc2)cc1 ; c1ccc(-c2ccc(-c3sc(-c4c(-c5ccccc5)ccc5c4sc4ccccc45)c4ccccc34)cc2)cc1 ; c1ccc(-c2ccc(-c3sc(-c4c(-c5ccccc5)ccc5c6ccccc6n(-c6ccccc6)c45)c4ccccc34)cc2)cc1 ; c1ccc(-c2ccccc2-c2sc(-c3ccc(-c4ccccc4)c4ccccc34)c3ccccc23)cc1 ; c1ccc(-c2ccccc2-c2sc(-c3cccc4c(-c5ccccc5)cccc34)c3ccccc23)cc1]

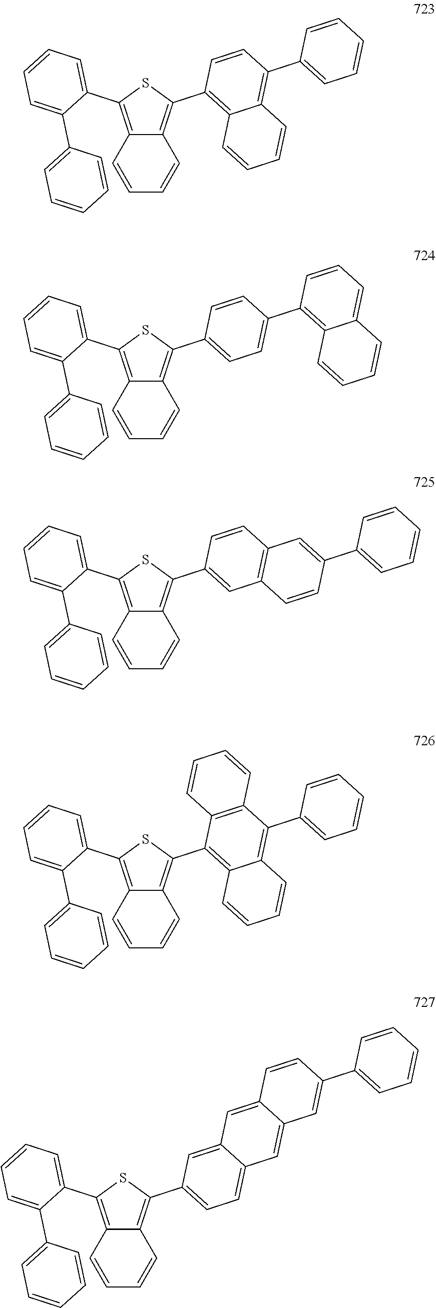 [SMILES: c1ccc(-c2ccccc2-c2sc(-c3ccc(-c4ccccc4)c4ccccc34)c3ccccc23)cc1 ; c1ccc(-c2ccccc2-c2sc(-c3ccc(-c4cccc5ccccc45)cc3)c3ccccc23)cc1 ; c1ccc(-c2ccc3cc(-c4sc(-c5ccccc5-c5ccccc5)c5ccccc45)ccc3c2)cc1 ; c1ccc(-c2ccccc2-c2sc(-c3c4ccccc4c(-c4ccccc4)c4ccccc34)c3ccccc23)cc1 ; c1ccc(-c2ccc3cc4cc(-c5sc(-c6ccccc6-c6ccccc6)c6ccccc56)ccc4cc3c2)cc1]

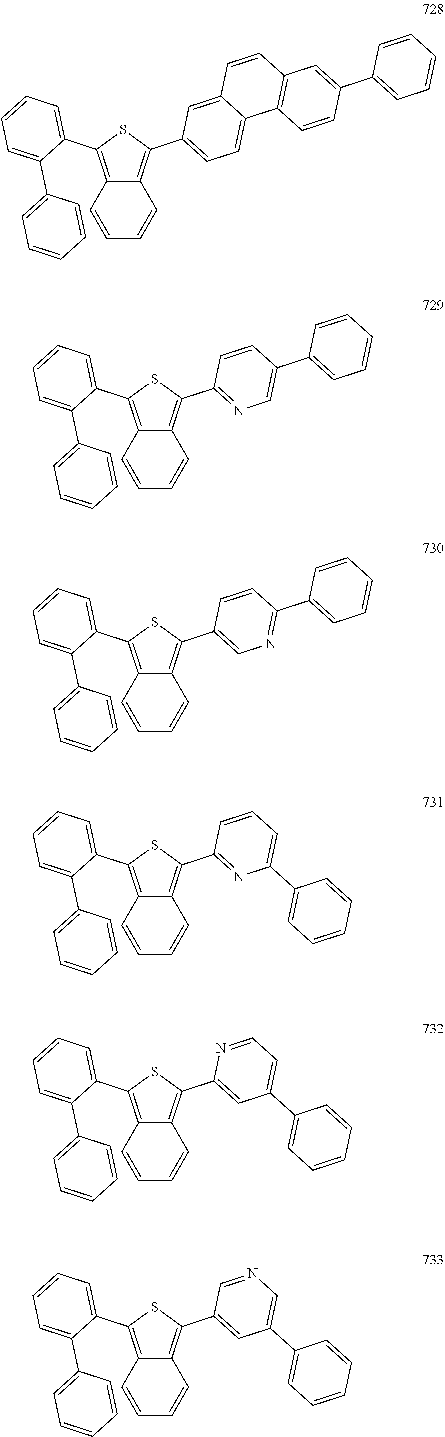 [SMILES: c1ccc(-c2ccc3c(ccc4cc(-c5sc(-c6ccccc6-c6ccccc6)c6ccccc56)ccc43)c2)cc1 ; c1ccc(-c2ccc(-c3sc(-c4ccccc4-c4ccccc4)c4ccccc34)nc2)cc1 ; c1ccc(-c2ccc(-c3sc(-c4ccccc4-c4ccccc4)c4ccccc34)cn2)cc1 ; c1ccc(-c2cccc(-c3sc(-c4ccccc4-c4ccccc4)c4ccccc34)n2)cc1 ; c1ccc(-c2ccnc(-c3sc(-c4ccccc4-c4ccccc4)c4ccccc34)c2)cc1 ; c1ccc(-c2cncc(-c3sc(-c4ccccc4-c4ccccc4)c4ccccc34)c2)cc1]

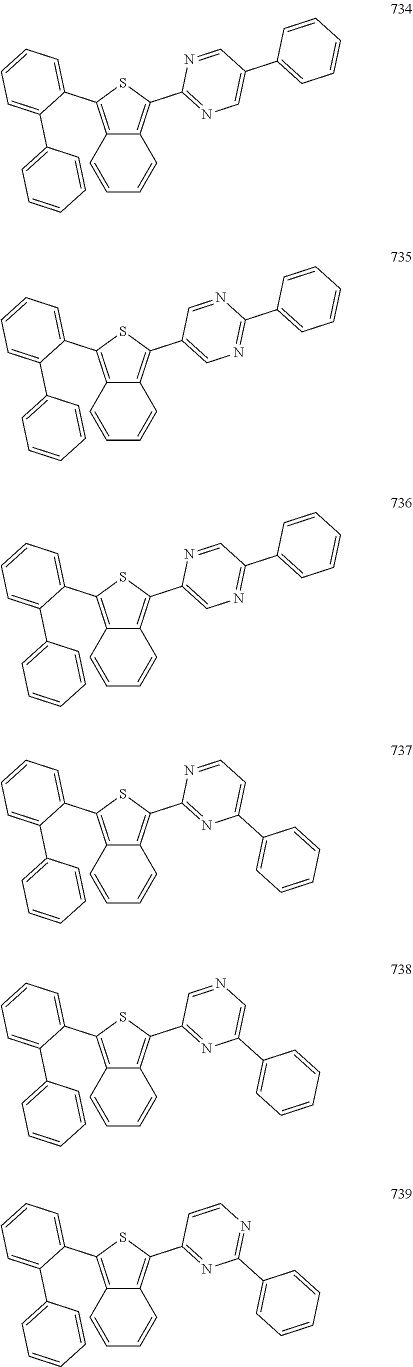 [SMILES: c1ccc(-c2cnc(-c3sc(-c4ccccc4-c4ccccc4)c4ccccc34)nc2)cc1 ; c1ccc(-c2ncc(-c3sc(-c4ccccc4-c4ccccc4)c4ccccc34)cn2)cc1 ; c1ccc(-c2cnc(-c3sc(-c4ccccc4-c4ccccc4)c4ccccc34)cn2)cc1 ; c1ccc(-c2ccnc(-c3sc(-c4ccccc4-c4ccccc4)c4ccccc34)n2)cc1 ; c1ccc(-c2cncc(-c3sc(-c4ccccc4-c4ccccc4)c4ccccc34)n2)cc1 ; c1ccc(-c2nccc(-c3sc(-c4ccccc4-c4ccccc4)c4ccccc34)n2)cc1]

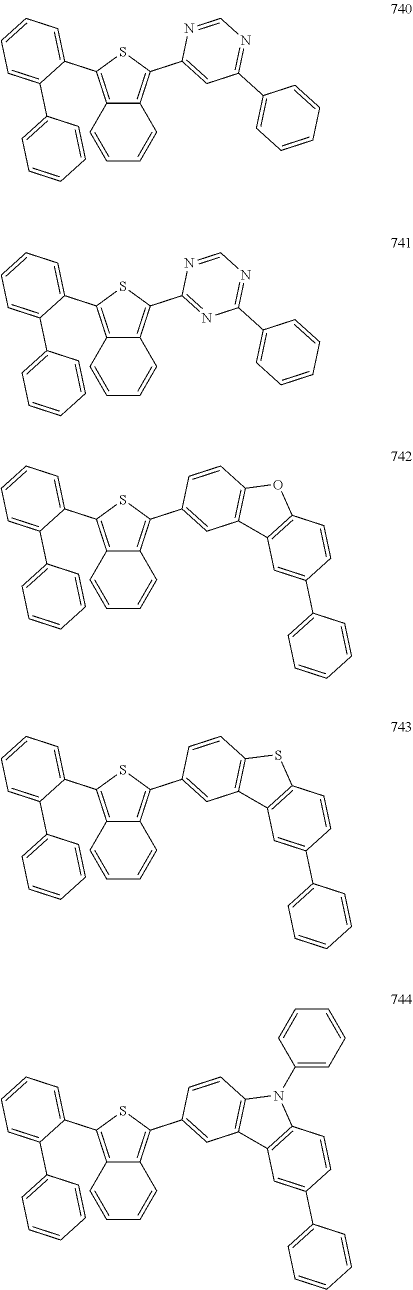 [SMILES: c1ccc(-c2cc(-c3sc(-c4ccccc4-c4ccccc4)c4ccccc34)ncn2)cc1 ; c1ccc(-c2ncnc(-c3sc(-c4ccccc4-c4ccccc4)c4ccccc34)n2)cc1 ; c1ccc(-c2ccc3oc4ccc(-c5sc(-c6ccccc6-c6ccccc6)c6ccccc56)cc4c3c2)cc1 ; c1ccc(-c2ccc3sc4ccc(-c5sc(-c6ccccc6-c6ccccc6)c6ccccc56)cc4c3c2)cc1 ; c1ccc(-c2ccc3c(c2)c2cc(-c4sc(-c5ccccc5-c5ccccc5)c5ccccc45)ccc2n3-c2ccccc2)cc1]

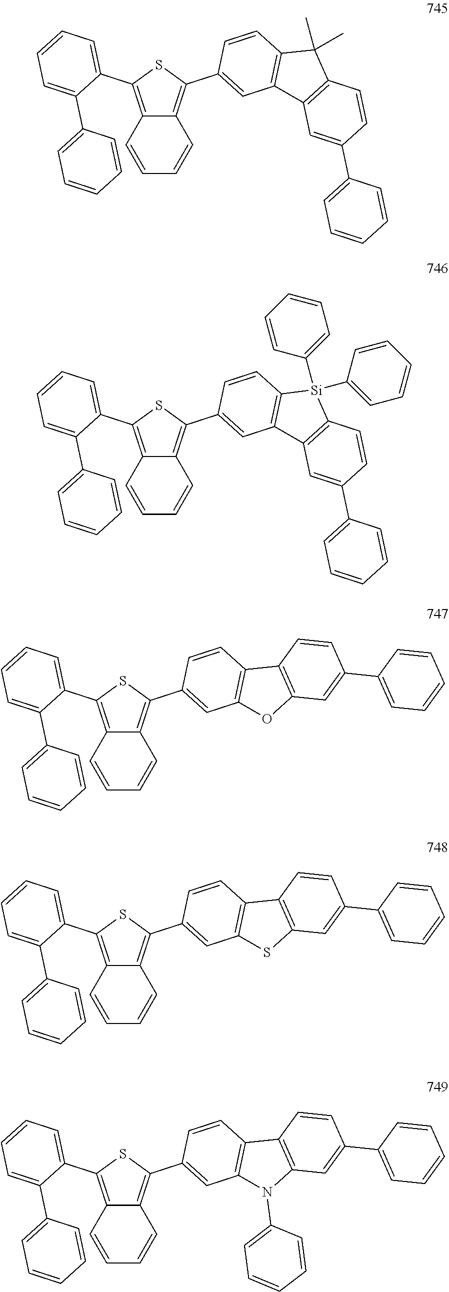 [SMILES: CC1(C)c2ccc(-c3ccccc3)cc2-c2cc(-c3sc(-c4ccccc4-c4ccccc4)c4ccccc34)ccc21 ; c1ccc(-c2ccc3c(c2)-c2cc(-c4sc(-c5ccccc5-c5ccccc5)c5ccccc45)ccc2[Si]3(c2ccccc2)c2ccccc2)cc1 ; c1ccc(-c2ccc3c(c2)oc2cc(-c4sc(-c5ccccc5-c5ccccc5)c5ccccc45)ccc23)cc1 ; c1ccc(-c2ccc3c(c2)sc2cc(-c4sc(-c5ccccc5-c5ccccc5)c5ccccc45)ccc23)cc1 ; c1ccc(-c2ccc3c4ccc(-c5sc(-c6ccccc6-c6ccccc6)c6ccccc56)cc4n(-c4ccccc4)c3c2)cc1]

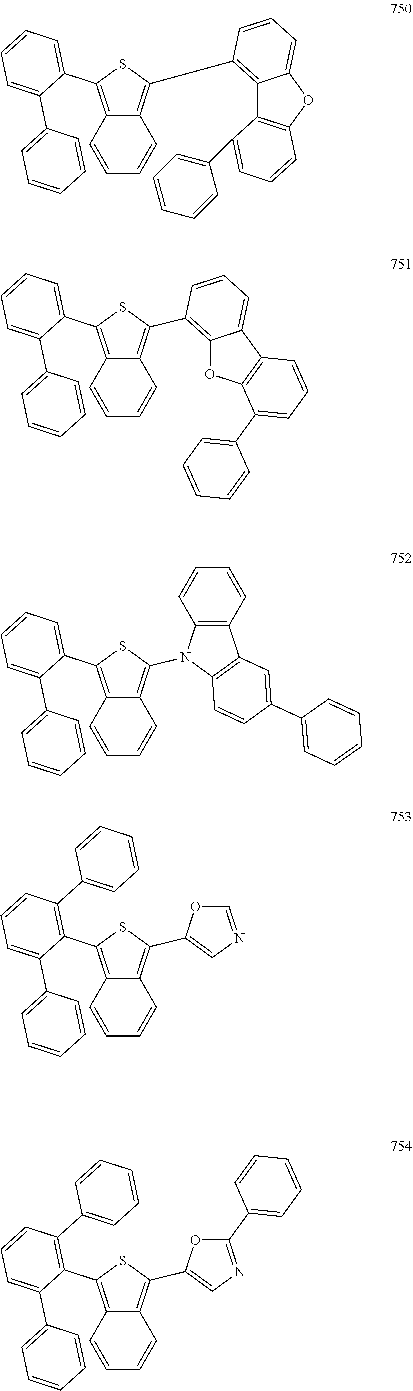 [SMILES: c1ccc(-c2ccccc2-c2sc(-c3cccc4oc5cccc(-c6ccccc6)c5c34)c3ccccc23)cc1 ; c1ccc(-c2ccccc2-c2sc(-c3cccc4c3oc3c(-c5ccccc5)cccc34)c3ccccc23)cc1 ; c1ccc(-c2ccc3c(c2)c2ccccc2n3-c2sc(-c3ccccc3-c3ccccc3)c3ccccc23)cc1 ; c1ccc(-c2cccc(-c3ccccc3)c2-c2sc(-c3cnco3)c3ccccc23)cc1 ; c1ccc(-c2ncc(-c3sc(-c4c(-c5ccccc5)cccc4-c4ccccc4)c4ccccc34)o2)cc1]

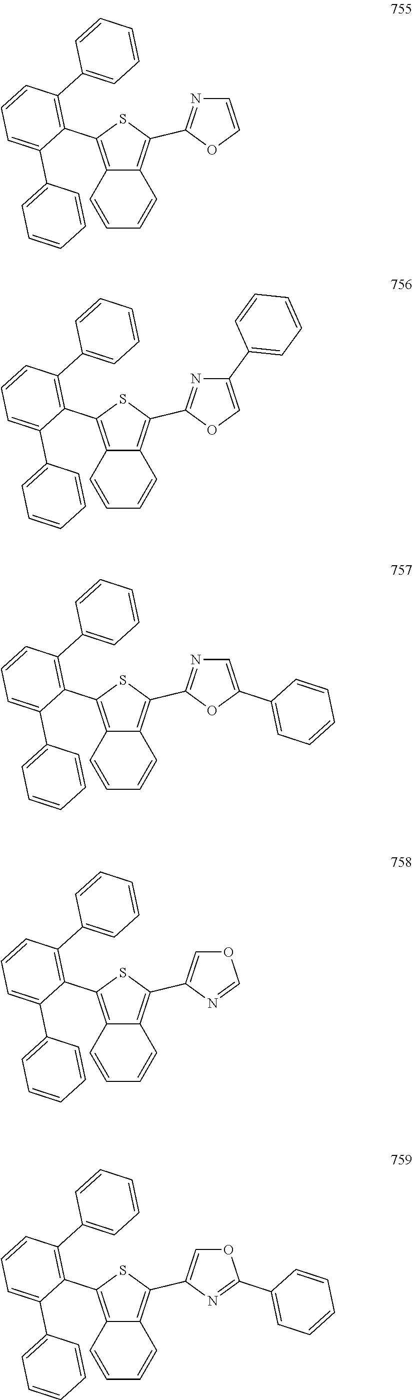 [SMILES: c1ccc(-c2cccc(-c3ccccc3)c2-c2sc(-c3ncco3)c3ccccc23)cc1 ; c1ccc(-c2coc(-c3sc(-c4c(-c5ccccc5)cccc4-c4ccccc4)c4ccccc34)n2)cc1 ; c1ccc(-c2cnc(-c3sc(-c4c(-c5ccccc5)cccc4-c4ccccc4)c4ccccc34)o2)cc1 ; c1ccc(-c2cccc(-c3ccccc3)c2-c2sc(-c3cocn3)c3ccccc23)cc1 ; c1ccc(-c2nc(-c3sc(-c4c(-c5ccccc5)cccc4-c4ccccc4)c4ccccc34)co2)cc1]

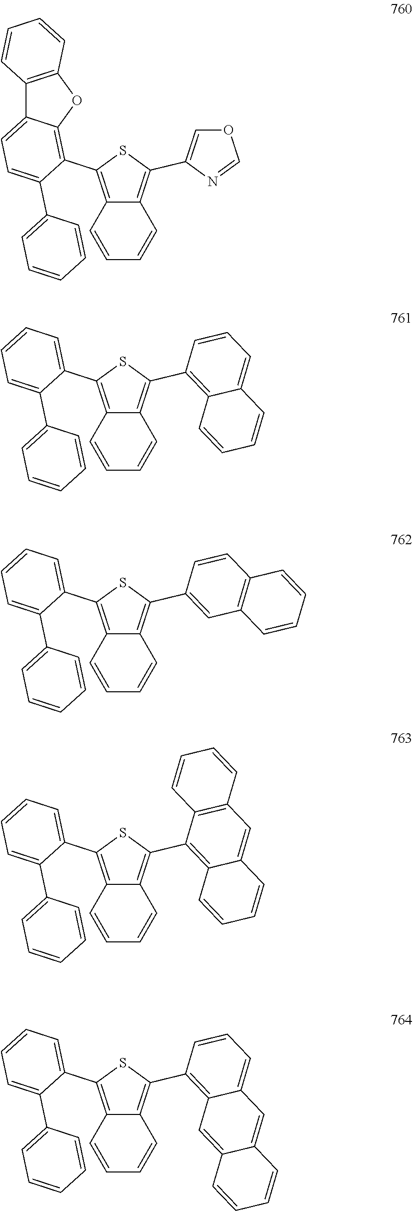 [SMILES: c1ccc(-c2ccc3c(oc4ccccc43)c2-c2sc(-c3cocn3)c3ccccc23)cc1 ; c1ccc(-c2ccccc2-c2sc(-c3cccc4ccccc34)c3ccccc23)cc1 ; c1ccc(-c2ccccc2-c2sc(-c3ccc4ccccc4c3)c3ccccc23)cc1 ; c1ccc(-c2ccccc2-c2sc(-c3c4ccccc4cc4ccccc34)c3ccccc23)cc1 ; c1ccc(-c2ccccc2-c2sc(-c3cccc4cc5ccccc5cc34)c3ccccc23)cc1]

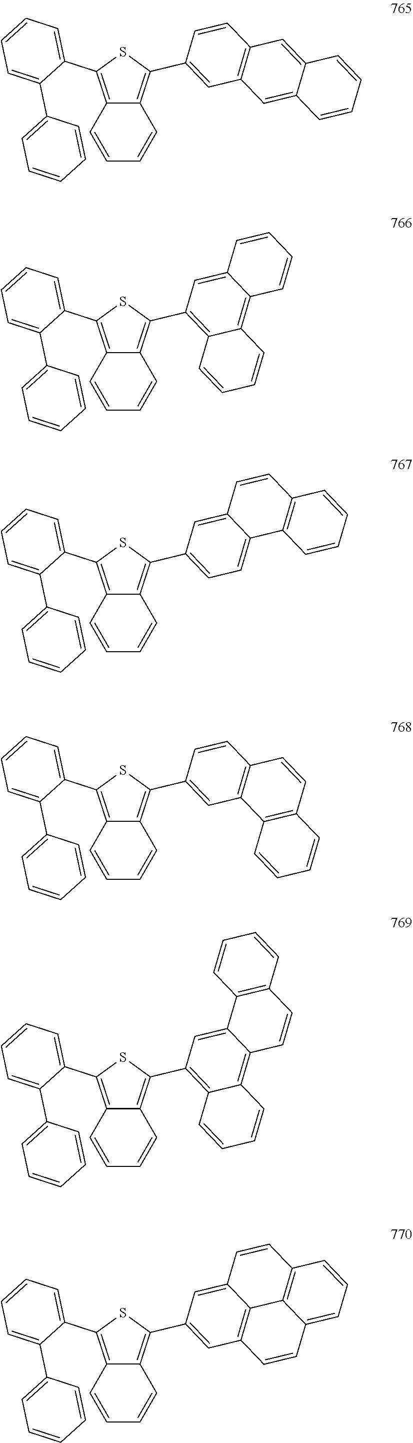 [SMILES: c1ccc(-c2ccccc2-c2sc(-c3ccc4cc5ccccc5cc4c3)c3ccccc23)cc1 ; c1ccc(-c2ccccc2-c2sc(-c3cc4ccccc4c4ccccc34)c3ccccc23)cc1 ; c1ccc(-c2ccccc2-c2sc(-c3ccc4c(ccc5ccccc54)c3)c3ccccc23)cc1 ; c1ccc(-c2ccccc2-c2sc(-c3ccc4ccc5ccccc5c4c3)c3ccccc23)cc1 ; c1ccc(-c2ccccc2-c2sc(-c3cc4c5ccccc5ccc4c4ccccc34)c3ccccc23)cc1 ; c1ccc(-c2ccccc2-c2sc(-c3cc4ccc5cccc6ccc(c3)c4c56)c3ccccc23)cc1]

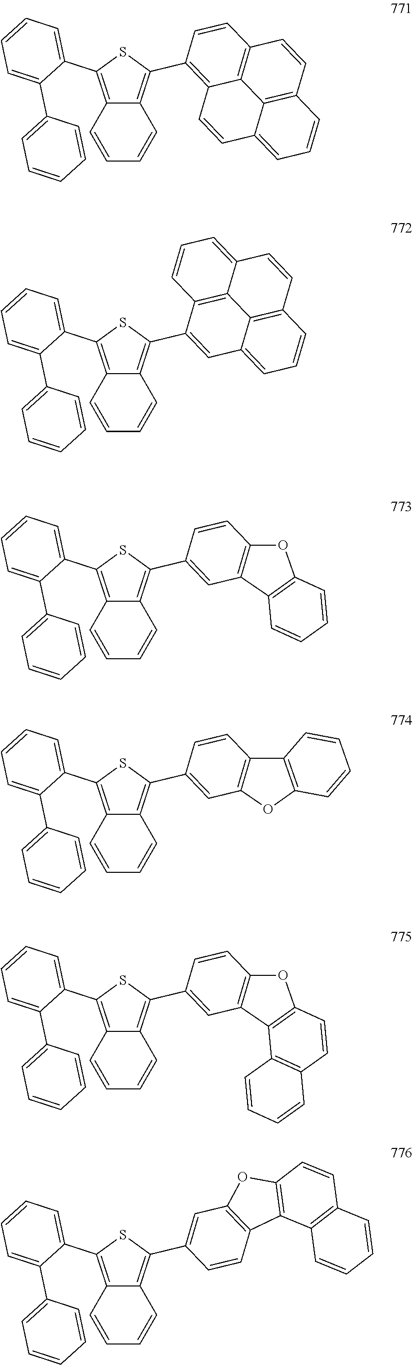

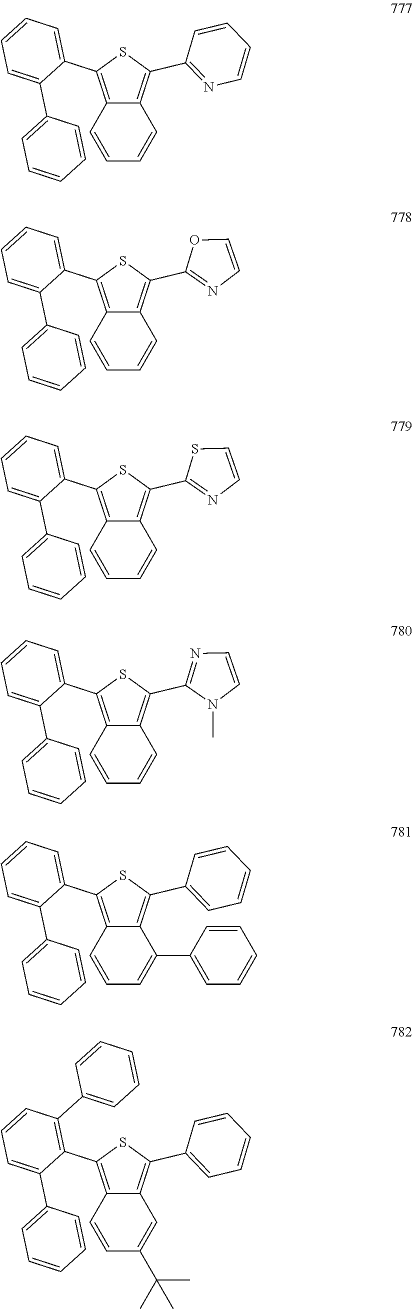 [SMILES: c1ccc(-c2ccccc2-c2sc(-c3ccccn3)c3ccccc23)cc1 ; c1ccc(-c2ccccc2-c2sc(-c3ncco3)c3ccccc23)cc1 ; c1ccc(-c2ccccc2-c2sc(-c3nccs3)c3ccccc23)cc1 ; Cn1ccnc1-c1sc(-c2ccccc2-c2ccccc2)c2ccccc12 ; c1ccc(-c2ccccc2-c2sc(-c3ccccc3)c3c(-c4ccccc4)cccc23)cc1 ; CC(C)(C)c1ccc2c(-c3c(-c4ccccc4)cccc3-c3ccccc3)sc(-c3ccccc3)c2c1]

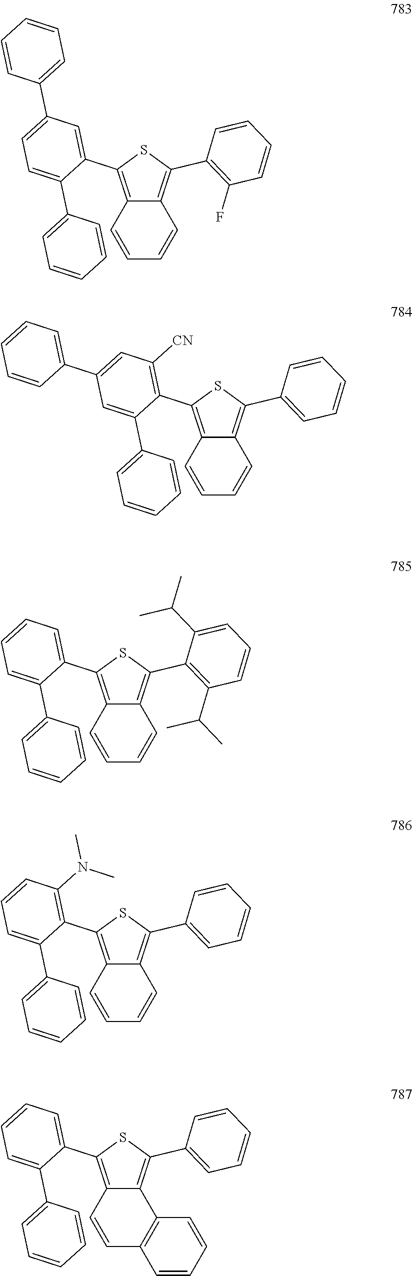 [SMILES: Fc1ccccc1-c1sc(-c2cc(-c3ccccc3)ccc2-c2ccccc2)c2ccccc12 ; N#Cc1cc(-c2ccccc2)cc(-c2ccccc2)c1-c1sc(-c2ccccc2)c2ccccc12 ; CC(C)c1cccc(C(C)C)c1-c1sc(-c2ccccc2-c2ccccc2)c2ccccc12 ; CN(C)c1cccc(-c2ccccc2)c1-c1sc(-c2ccccc2)c2ccccc12 ; c1ccc(-c2ccccc2-c2sc(-c3ccccc3)c3c2ccc2ccccc23)cc1]

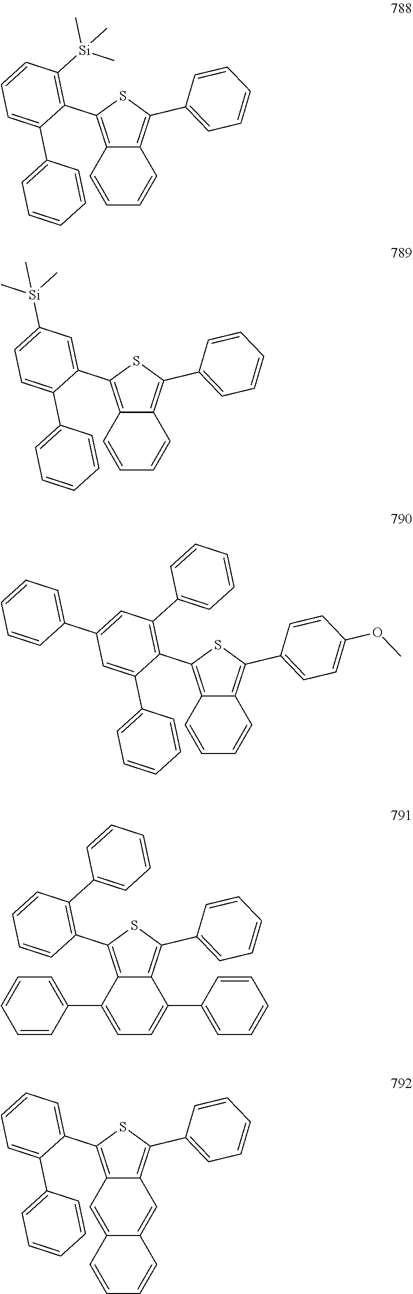 [SMILES: C[Si](C)(C)c1cccc(-c2ccccc2)c1-c1sc(-c2ccccc2)c2ccccc12 ; C[Si](C)(C)c1ccc(-c2ccccc2)c(-c2sc(-c3ccccc3)c3ccccc23)c1 ; COc1ccc(-c2sc(-c3c(-c4ccccc4)cc(-c4ccccc4)cc3-c3ccccc3)c3ccccc23)cc1 ; c1ccc(-c2ccccc2-c2sc(-c3ccccc3)c3c(-c4ccccc4)ccc(-c4ccccc4)c23)cc1 ; c1ccc(-c2ccccc2-c2sc(-c3ccccc3)c3cc4ccccc4cc23)cc1]

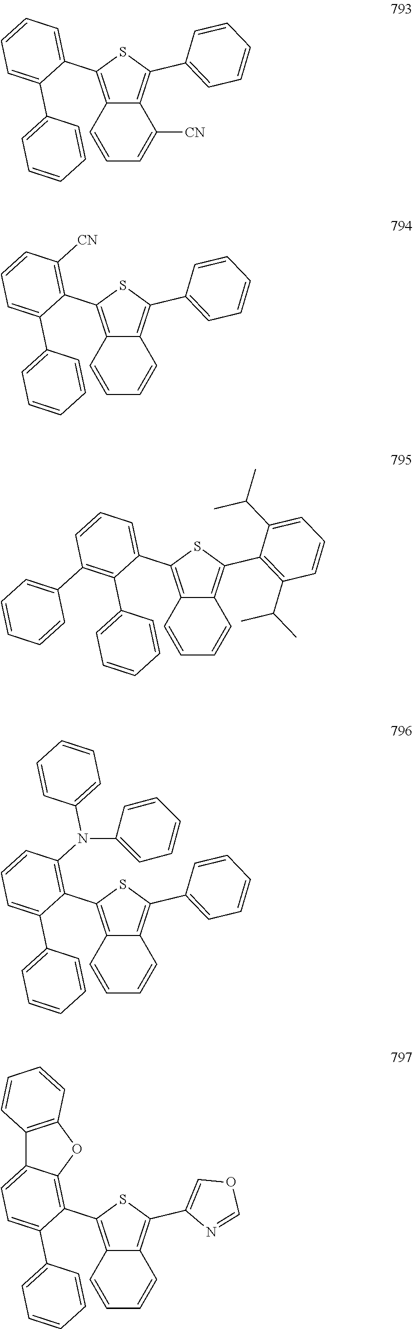 [SMILES: N#Cc1cccc2c(-c3ccccc3-c3ccccc3)sc(-c3ccccc3)c12 ; N#Cc1cccc(-c2ccccc2)c1-c1sc(-c2ccccc2)c2ccccc12 ; CC(C)c1cccc(C(C)C)c1-c1sc(-c2cccc(-c3ccccc3)c2-c2ccccc2)c2ccccc12 ; c1ccc(-c2cccc(N(c3ccccc3)c3ccccc3)c2-c2sc(-c3ccccc3)c3ccccc23)cc1 ; c1ccc(-c2ccc3c(oc4ccccc43)c2-c2sc(-c3cocn3)c3ccccc23)cc1]

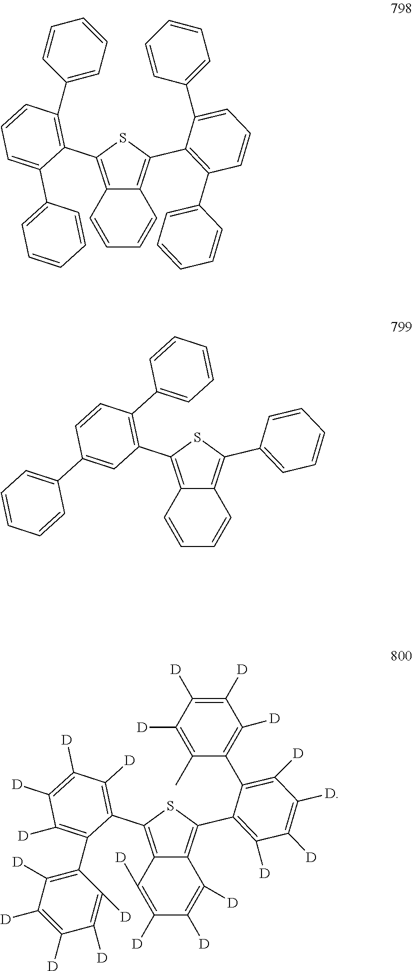

In an embodiment, the fluorescent host may be represented by Formula FH-3:

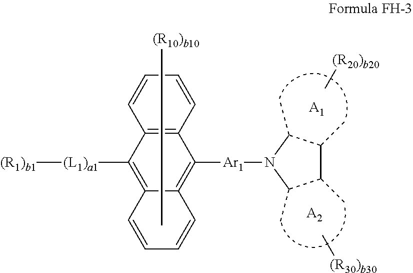

wherein, in Formula FH-3,
Ar₁ may be a group represented by Formula 2,

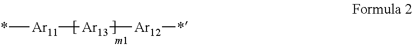

Ar₁ may include at least one cyano group,
A₁ and A₂ may each independently be a C₅-C₃₀ carbocyclic group or a C₁-C₃₀ heterocyclic group,
L₁ may be an unsubstituted or substituted C₅-C₃₀ carbocyclic group or an unsubstituted or substituted C₁-C₃₀ heterocyclic group,
a1 may be 0, 1, 2, or 3,
when a1 is 2 or greater, two or more of L₁₁ may be identical to or different from each other,
m1 may be 0, 1, 2, or 3,
Ar₁₁ may be a group represented by Formula 4, Ar₁₂ may be a group represented by Formula 5, and Ar₁₃ may be a group represented by Formula 6:

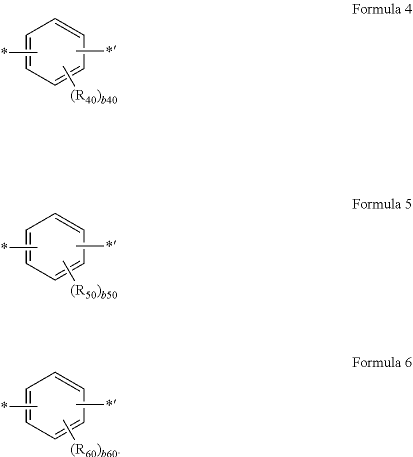

In Formulae 4 to 6,
R₁, R₁₀, R₂₀, R₃₀, R₄₀, R₅₀, and R₆₀ may each independently be hydrogen, deuterium, —F, —Cl, —Br, —I, —SF₅, a hydroxyl group, a cyano group, a nitro group, an amidino group, a hydrazine group, a hydrazone group, a carboxylic acid group or a salt thereof, a sulfonic acid group or a salt thereof, a phosphoric acid group or a salt thereof, a substituted or unsubstituted C₁-C₆₀ alkyl group, a substituted or unsubstituted C₂-C₆₀ alkenyl group, a substituted or unsubstituted C₂-C₆₀ alkynyl group, a substituted or unsubstituted C₁-C₆₀ alkoxy group, a substituted or unsubstituted C₃-C₁₀ cycloalkyl group, a substituted or unsubstituted heterocycloalkyl group, a substituted or unsubstituted C₃-C₁₀ cycloalkenyl group, a substituted or unsubstituted C₁-C₁₀heterocycloalkenyl group, a substituted or unsubstituted C₆-C₆₀ aryl group, a substituted or unsubstituted C₆-C₆₀ aryloxy group, a substituted or unsubstituted C₆-C₆₀ arylthio group, a substituted or unsubstituted C₁-C₆₀ heteroaryl group, a substituted or unsubstituted C₁-C₆₀ heteroaryloxy group, a substituted or unsubstituted C₁-C₆₀ heteroarylthio group, a substituted or unsubstituted monovalent non-aromatic condensed polycyclic group, a substituted or unsubstituted monovalent non-aromatic condensed heteropolycyclic group, —N(Q₁)(Q₂), —Si(Q₃)(Q₄)(Q₅), —B(Q₆)(Q₇), or —P(═O)(Q₈)(Q₉),
b1 may be an integer from 1 to 5,
when b1 is 2 or greater, two or more of R₁ may be identical to or different from each other,
b10 may be an integer from 1 to 8, when b10 is 2 or greater, two or more of R₁₀ may be identical to or different from each other,
b20 and b30 may each independently be an integer from 1 to 4,
when b20 is 2 or greater, two or more of R₂₀ may be identical to or different from each other, and when b30 2 or greater, two or more of R₃₀ may be identical to or different from each other,
b40, b50, and b60 may each independently be an integer from 1 to 4,
when b40 is 2 or greater, two or more of R₄₀ may be identical to or different from each other, when b50 is 2 or greater, two or more of R₅₀ may be identical to or different from each other, and when b60 is 2 or greater, two or more R₆₀ may be identical to or different from each other, and
* and *1 each indicate a binding site to a neighboring atom.

In an embodiment, the fluorescent host represented by Formula FH-3 may be a compound of Group FH3, but embodiments of the present disclosure are not limited thereto:

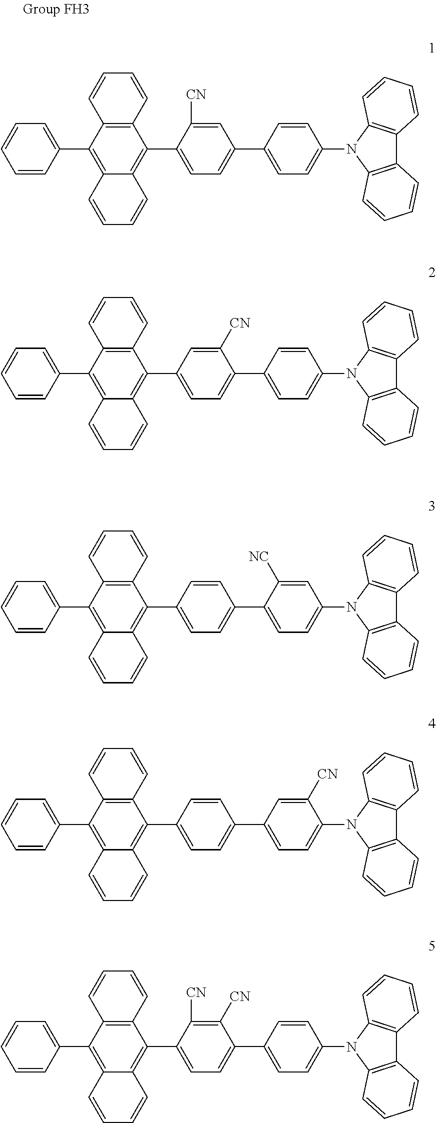 [SMILES: N#Cc1cc(-c2ccc(-n3c4ccccc4c4ccccc43)cc2)ccc1-c1c2ccccc2c(-c2ccccc2)c2ccccc12 ; N#Cc1cc(-c2c3ccccc3c(-c3ccccc3)c3ccccc23)ccc1-c1ccc(-n2c3ccccc3c3ccccc32)cc1 ; N#Cc1cc(-n2c3ccccc3c3ccccc32)ccc1-c1ccc(-c2c3ccccc3c(-c3ccccc3)c3ccccc23)cc1 ; N#Cc1cc(-c2ccc(-c3c4ccccc4c(-c4ccccc4)c4ccccc34)cc2)ccc1-n1c2ccccc2c2ccccc21 ; N#Cc1c(-c2ccc(-n3c4ccccc4c4ccccc43)cc2)ccc(-c2c3ccccc3c(-c3ccccc3)c3ccccc23)c1C#N]

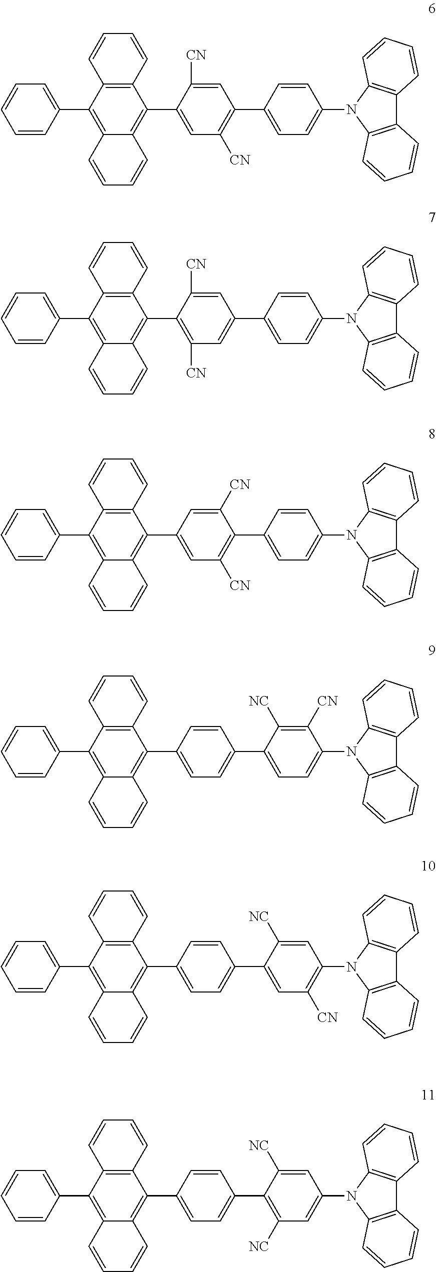

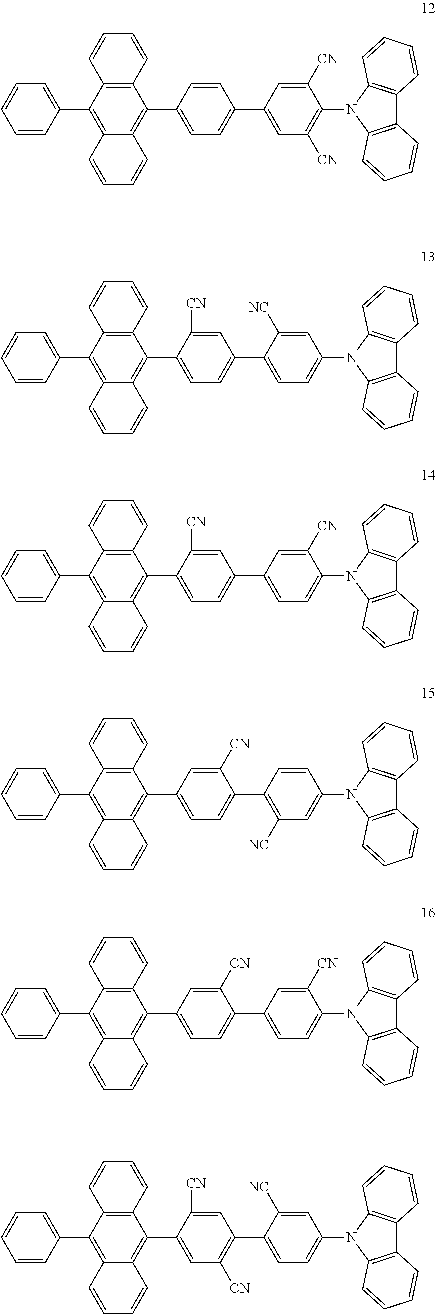

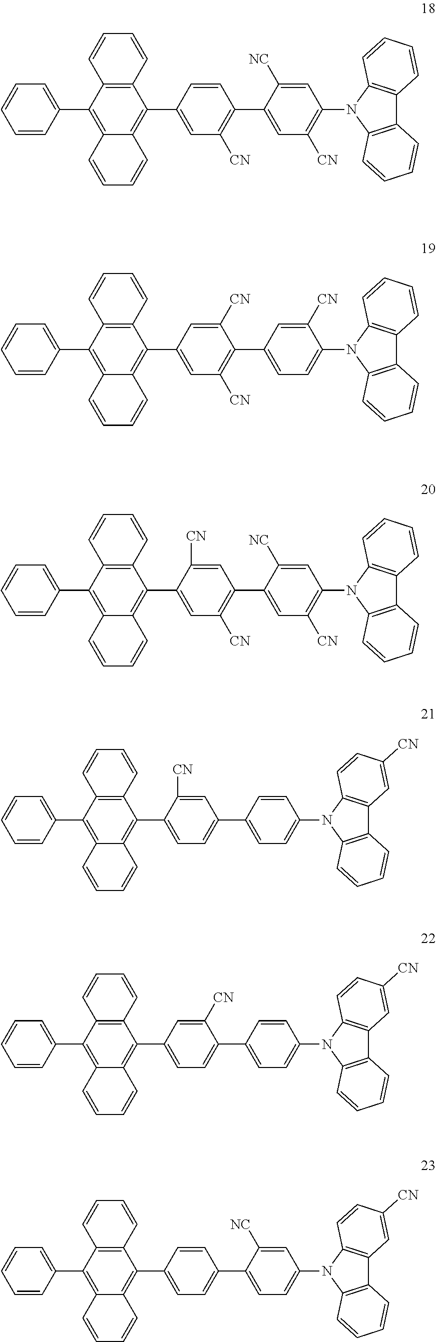

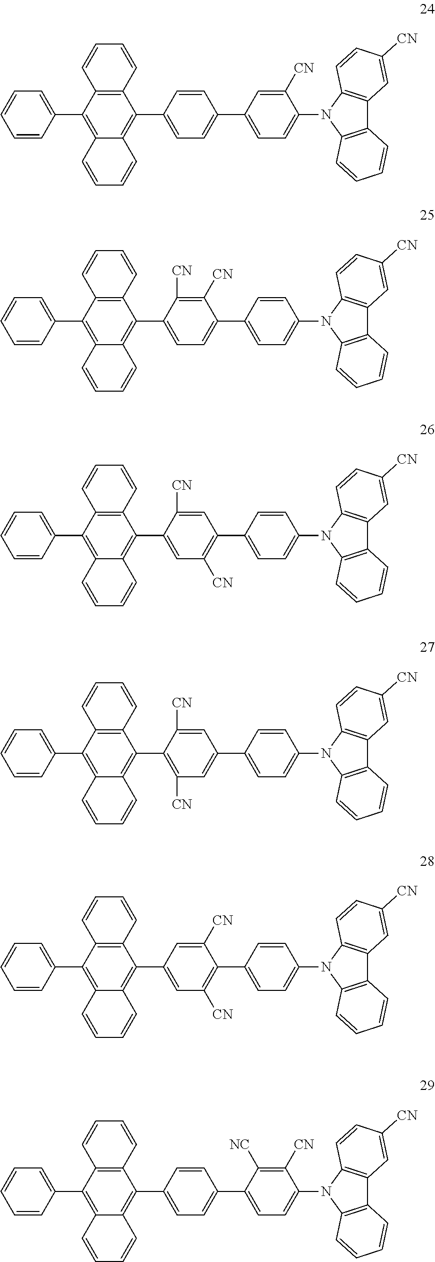

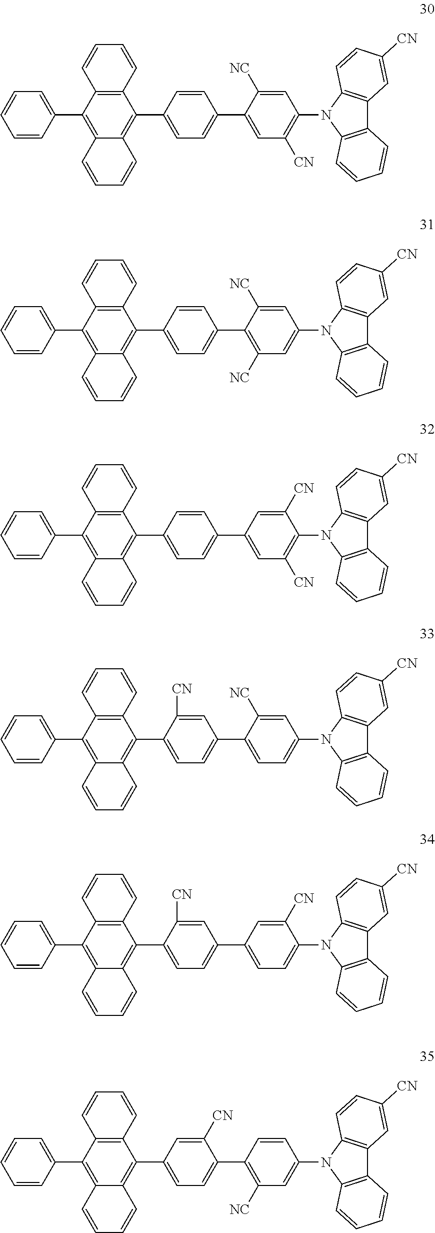

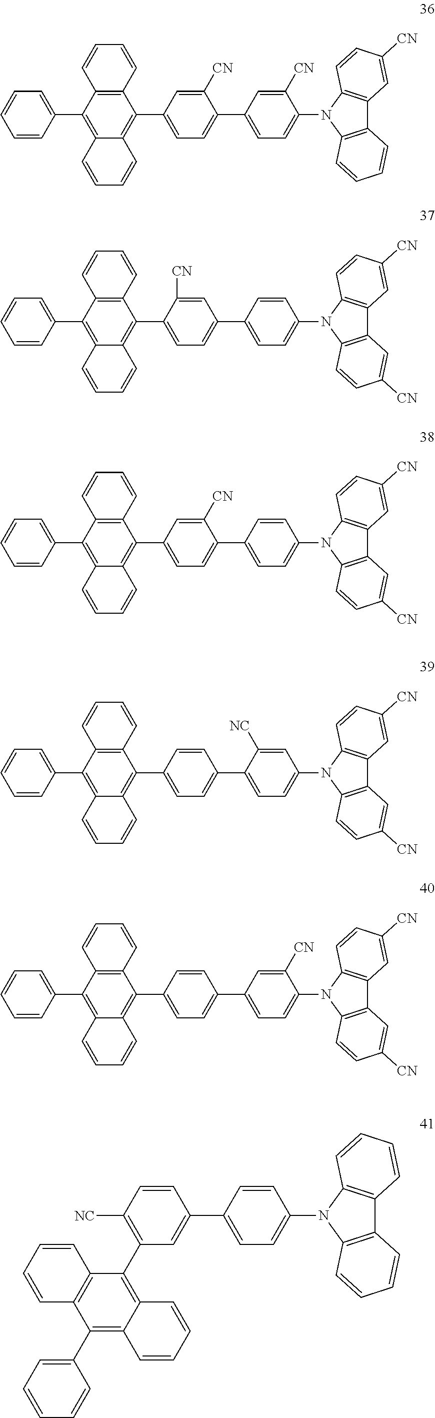

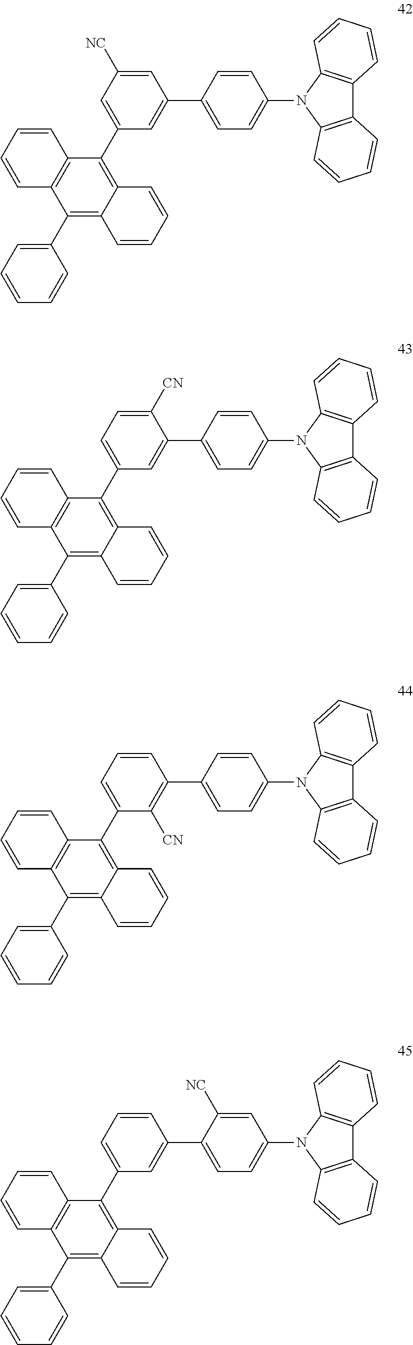 [SMILES: N#Cc1cc(-c2ccc(-n3c4ccccc4c4ccccc43)cc2)cc(-c2c3ccccc3c(-c3ccccc3)c3ccccc23)c1 ; N#Cc1ccc(-c2c3ccccc3c(-c3ccccc3)c3ccccc23)cc1-c1ccc(-n2c3ccccc3c3ccccc32)cc1 ; N#Cc1c(-c2ccc(-n3c4ccccc4c4ccccc43)cc2)cccc1-c1c2ccccc2c(-c2ccccc2)c2ccccc12 ; N#Cc1cc(-n2c3ccccc3c3ccccc32)ccc1-c1cccc(-c2c3ccccc3c(-c3ccccc3)c3ccccc23)c1]

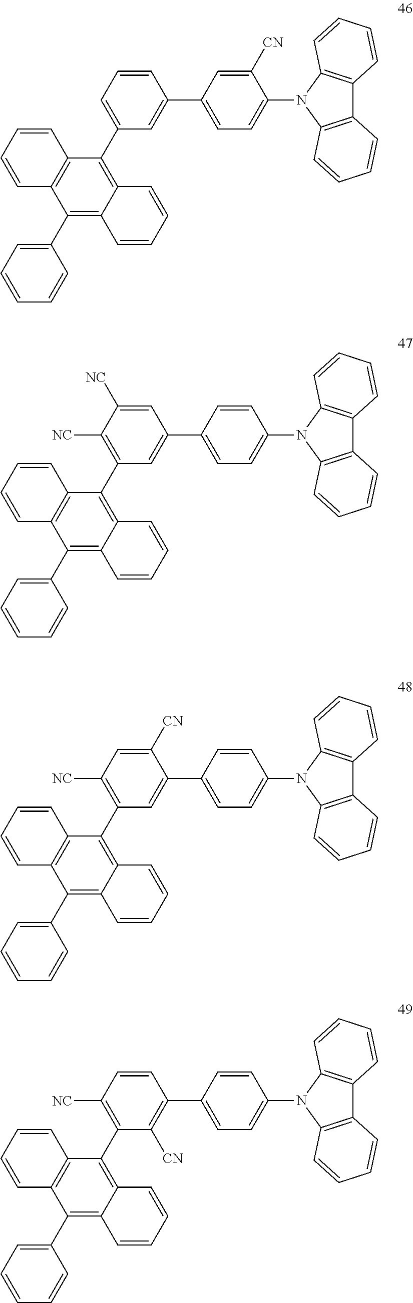 [SMILES: N#Cc1cc(-c2cccc(-c3c4ccccc4c(-c4ccccc4)c4ccccc34)c2)ccc1-n1c2ccccc2c2ccccc21 ; N#Cc1cc(-c2ccc(-n3c4ccccc4c4ccccc43)cc2)cc(-c2c3ccccc3c(-c3ccccc3)c3ccccc23)c1C#N ; N#Cc1cc(C#N)c(-c2c3ccccc3c(-c3ccccc3)c3ccccc23)cc1-c1ccc(-n2c3ccccc3c3ccccc32)cc1 ; N#Cc1ccc(-c2ccc(-n3c4ccccc4c4ccccc43)cc2)c(C#N)c1-c1c2ccccc2c(-c2ccccc2)c2ccccc12]

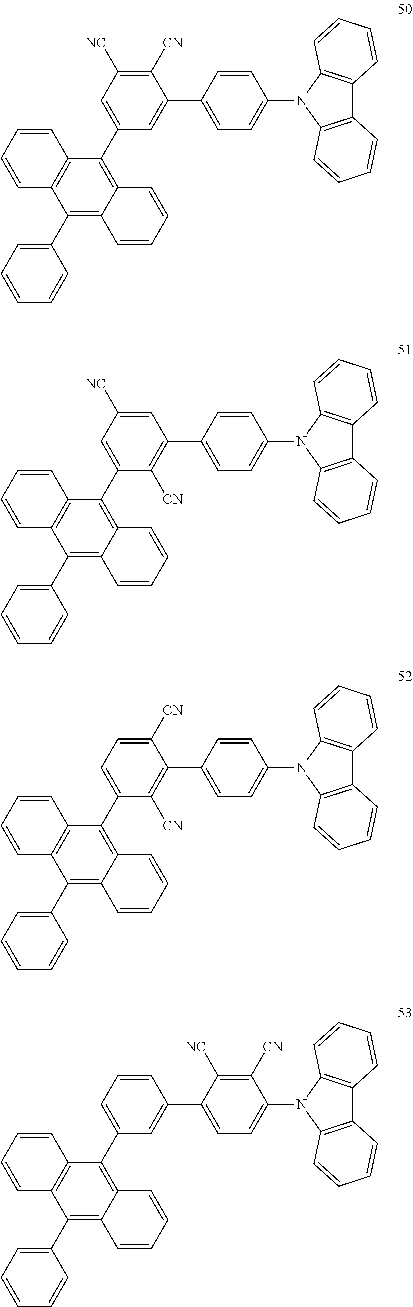 [SMILES: N#Cc1cc(-c2c3ccccc3c(-c3ccccc3)c3ccccc23)cc(-c2ccc(-n3c4ccccc4c4ccccc43)cc2)c1C#N ; N#Cc1cc(-c2ccc(-n3c4ccccc4c4ccccc43)cc2)c(C#N)c(-c2c3ccccc3c(-c3ccccc3)c3ccccc23)c1 ; N#Cc1ccc(-c2c3ccccc3c(-c3ccccc3)c3ccccc23)c(C#N)c1-c1ccc(-n2c3ccccc3c3ccccc32)cc1 ; N#Cc1c(-c2cccc(-c3c4ccccc4c(-c4ccccc4)c4ccccc34)c2)ccc(-n2c3ccccc3c3ccccc32)c1C#N]

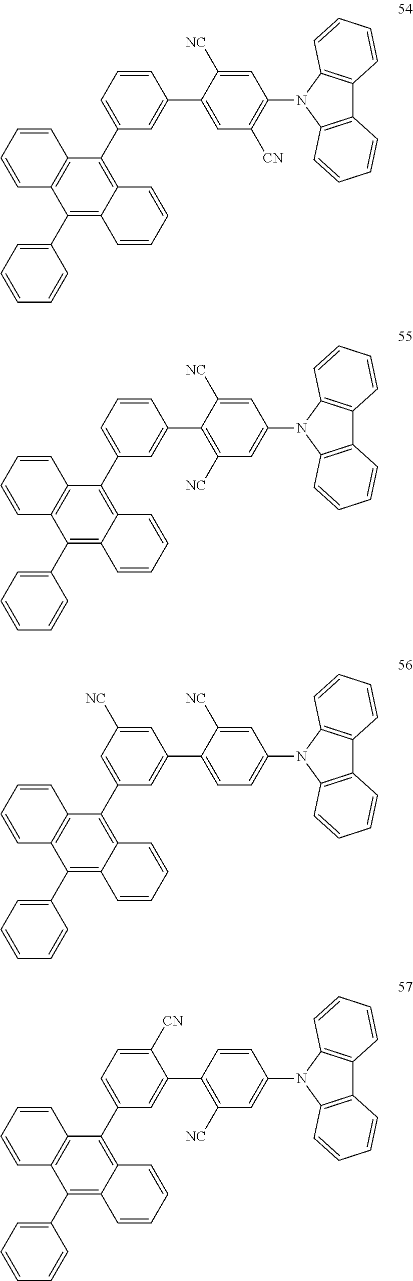 [SMILES: N#Cc1cc(-n2c3ccccc3c3ccccc32)c(C#N)cc1-c1cccc(-c2c3ccccc3c(-c3ccccc3)c3ccccc23)c1 ; N#Cc1cc(-n2c3ccccc3c3ccccc32)cc(C#N)c1-c1cccc(-c2c3ccccc3c(-c3ccccc3)c3ccccc23)c1 ; N#Cc1cc(-c2ccc(-n3c4ccccc4c4ccccc43)cc2C#N)cc(-c2c3ccccc3c(-c3ccccc3)c3ccccc23)c1 ; N#Cc1cc(-n2c3ccccc3c3ccccc32)ccc1-c1cc(-c2c3ccccc3c(-c3ccccc3)c3ccccc23)ccc1C#N]

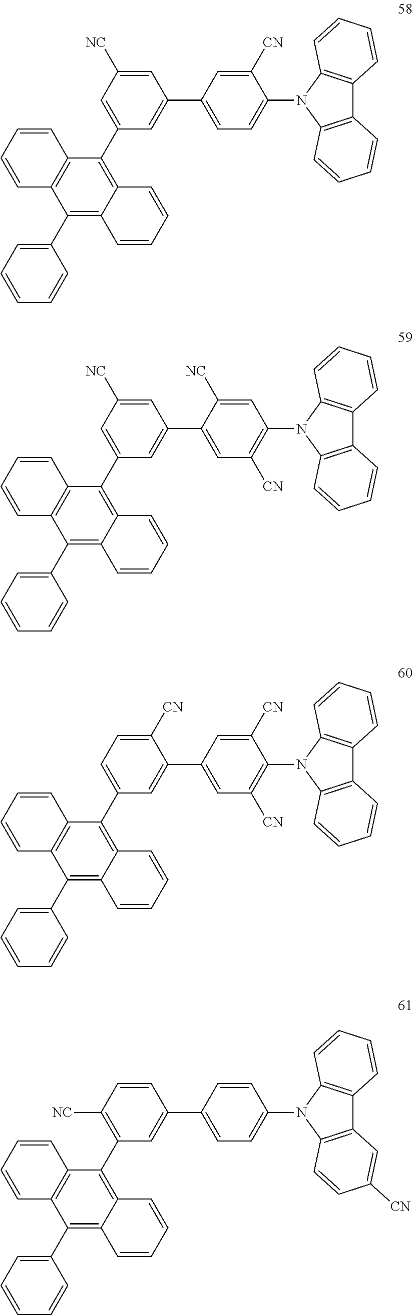 [SMILES: N#Cc1cc(-c2ccc(-n3c4ccccc4c4ccccc43)c(C#N)c2)cc(-c2c3ccccc3c(-c3ccccc3)c3ccccc23)c1 ; N#Cc1cc(-c2cc(C#N)c(-n3c4ccccc4c4ccccc43)cc2C#N)cc(-c2c3ccccc3c(-c3ccccc3)c3ccccc23)c1 ; N#Cc1ccc(-c2c3ccccc3c(-c3ccccc3)c3ccccc23)cc1-c1cc(C#N)c(-n2c3ccccc3c3ccccc32)c(C#N)c1 ; N#Cc1ccc2c(c1)c1ccccc1n2-c1ccc(-c2ccc(C#N)c(-c3c4ccccc4c(-c4ccccc4)c4ccccc34)c2)cc1]

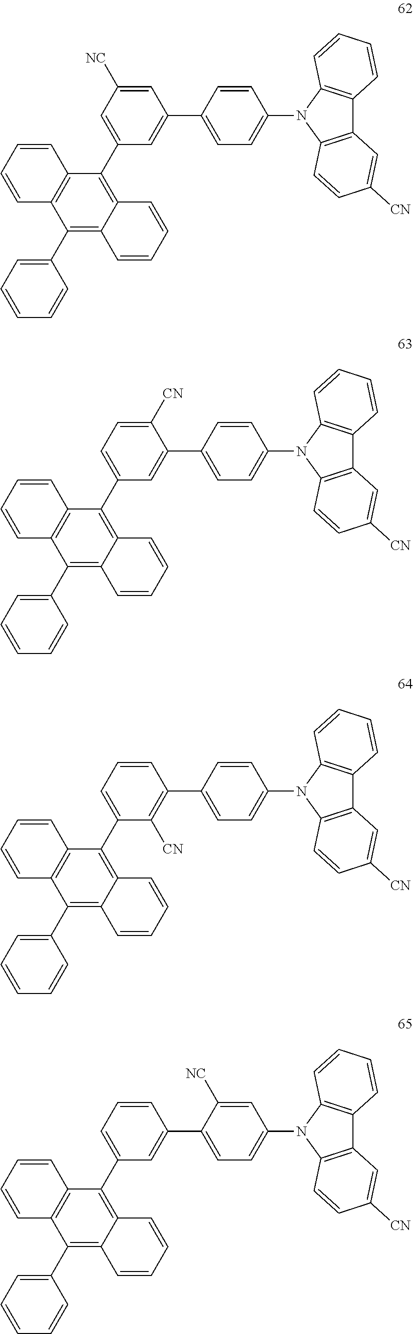 [SMILES: N#Cc1cc(-c2ccc(-n3c4ccccc4c4cc(C#N)ccc43)cc2)cc(-c2c3ccccc3c(-c3ccccc3)c3ccccc23)c1 ; N#Cc1ccc2c(c1)c1ccccc1n2-c1ccc(-c2cc(-c3c4ccccc4c(-c4ccccc4)c4ccccc34)ccc2C#N)cc1 ; N#Cc1ccc2c(c1)c1ccccc1n2-c1ccc(-c2cccc(-c3c4ccccc4c(-c4ccccc4)c4ccccc34)c2C#N)cc1 ; N#Cc1ccc2c(c1)c1ccccc1n2-c1ccc(-c2cccc(-c3c4ccccc4c(-c4ccccc4)c4ccccc34)c2)c(C#N)c1]

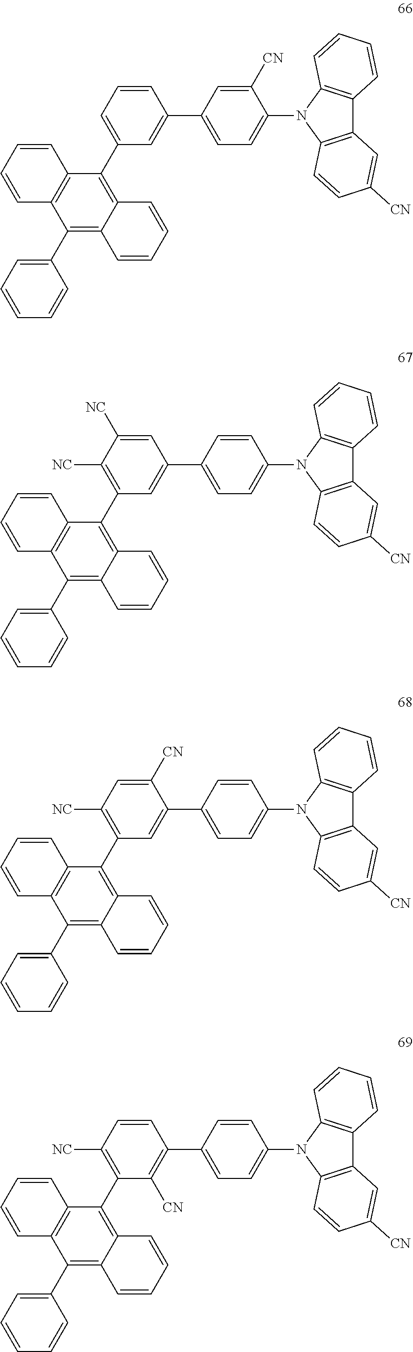 [SMILES: N#Cc1ccc2c(c1)c1ccccc1n2-c1ccc(-c2cccc(-c3c4ccccc4c(-c4ccccc4)c4ccccc34)c2)cc1C#N ; N#Cc1ccc2c(c1)c1ccccc1n2-c1ccc(-c2cc(C#N)c(C#N)c(-c3c4ccccc4c(-c4ccccc4)c4ccccc34)c2)cc1 ; N#Cc1ccc2c(c1)c1ccccc1n2-c1ccc(-c2cc(-c3c4ccccc4c(-c4ccccc4)c4ccccc34)c(C#N)cc2C#N)cc1 ; N#Cc1ccc2c(c1)c1ccccc1n2-c1ccc(-c2ccc(C#N)c(-c3c4ccccc4c(-c4ccccc4)c4ccccc34)c2C#N)cc1]

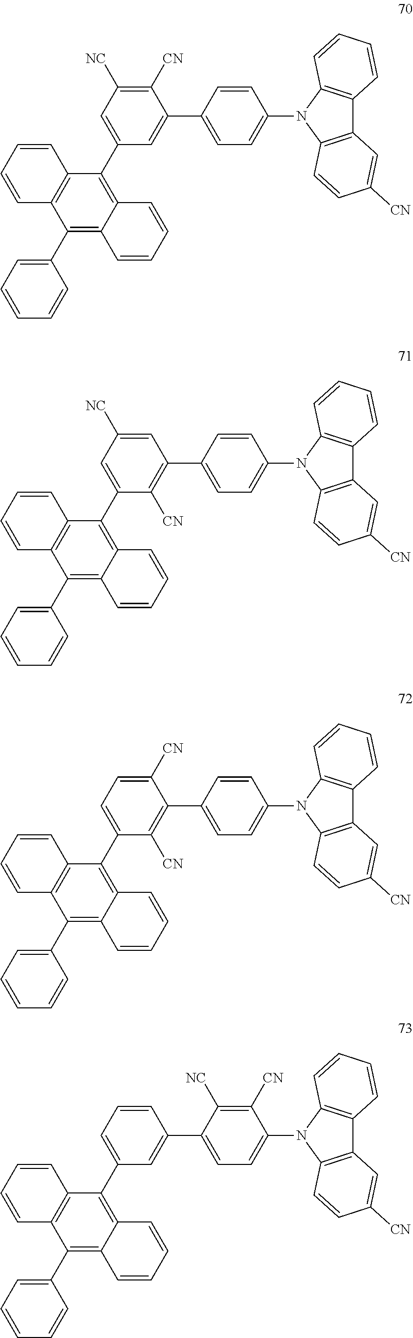 [SMILES: N#Cc1ccc2c(c1)c1ccccc1n2-c1ccc(-c2cc(-c3c4ccccc4c(-c4ccccc4)c4ccccc34)cc(C#N)c2C#N)cc1 ; N#Cc1cc(-c2ccc(-n3c4ccccc4c4cc(C#N)ccc43)cc2)c(C#N)c(-c2c3ccccc3c(-c3ccccc3)c3ccccc23)c1 ; N#Cc1ccc2c(c1)c1ccccc1n2-c1ccc(-c2c(C#N)ccc(-c3c4ccccc4c(-c4ccccc4)c4ccccc34)c2C#N)cc1 ; N#Cc1ccc2c(c1)c1ccccc1n2-c1ccc(-c2cccc(-c3c4ccccc4c(-c4ccccc4)c4ccccc34)c2)c(C#N)c1C#N]

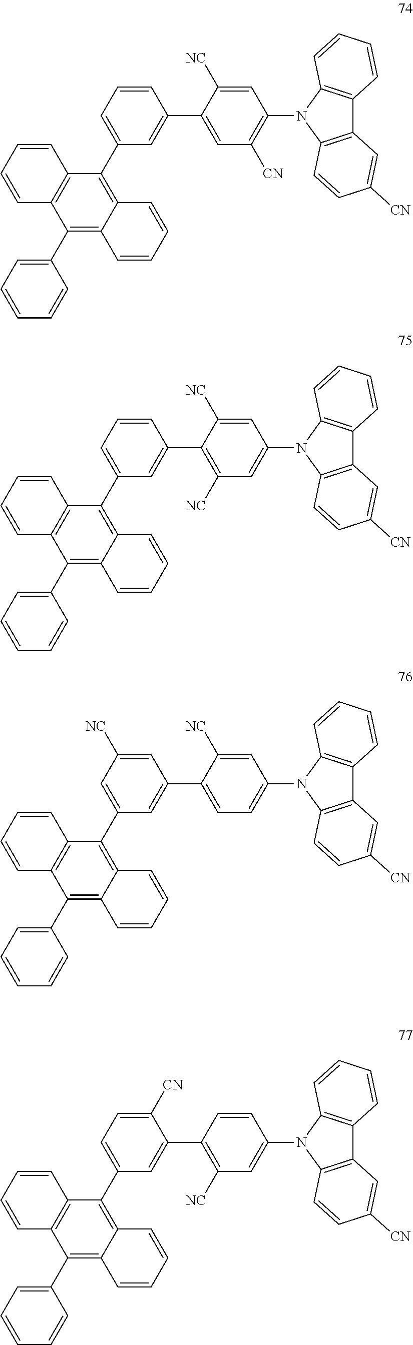 [SMILES: N#Cc1ccc2c(c1)c1ccccc1n2-c1cc(C#N)c(-c2cccc(-c3c4ccccc4c(-c4ccccc4)c4ccccc34)c2)cc1C#N ; N#Cc1ccc2c(c1)c1ccccc1n2-c1cc(C#N)c(-c2cccc(-c3c4ccccc4c(-c4ccccc4)c4ccccc34)c2)c(C#N)c1 ; N#Cc1cc(-c2ccc(-n3c4ccccc4c4cc(C#N)ccc43)cc2C#N)cc(-c2c3ccccc3c(-c3ccccc3)c3ccccc23)c1 ; N#Cc1ccc2c(c1)c1ccccc1n2-c1ccc(-c2cc(-c3c4ccccc4c(-c4ccccc4)c4ccccc34)ccc2C#N)c(C#N)c1]

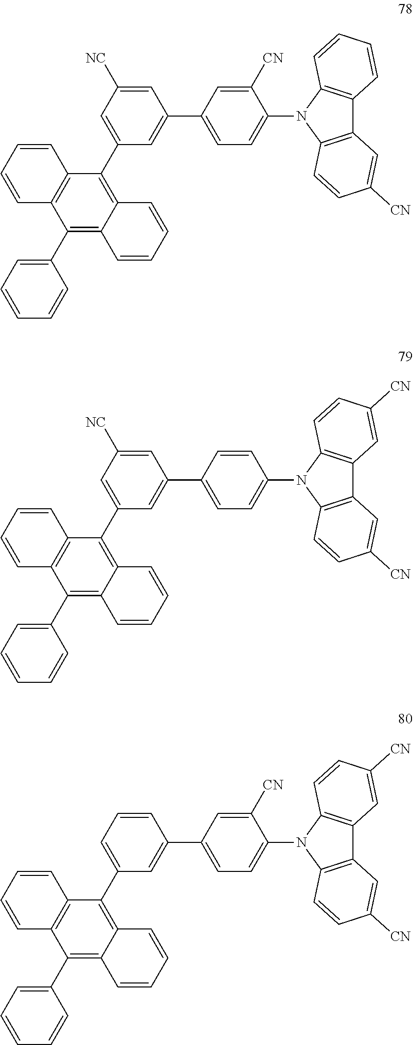 [SMILES: N#Cc1cc(-c2ccc(-n3c4ccccc4c4cc(C#N)ccc43)c(C#N)c2)cc(-c2c3ccccc3c(-c3ccccc3)c3ccccc23)c1 ; N#Cc1cc(-c2ccc(-n3c4ccc(C#N)cc4c4cc(C#N)ccc43)cc2)cc(-c2c3ccccc3c(-c3ccccc3)c3ccccc23)c1 ; N#Cc1ccc2c(c1)c1cc(C#N)ccc1n2-c1ccc(-c2cccc(-c3c4ccccc4c(-c4ccccc4)c4ccccc34)c2)cc1C#N]

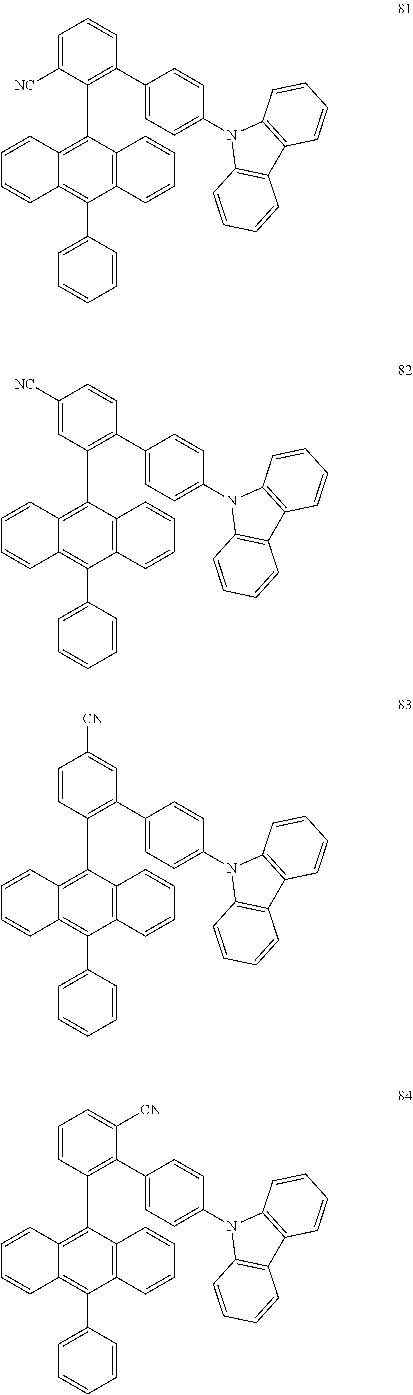 [SMILES: N#Cc1cccc(-c2ccc(-n3c4ccccc4c4ccccc43)cc2)c1-c1c2ccccc2c(-c2ccccc2)c2ccccc12 ; N#Cc1ccc(-c2ccc(-n3c4ccccc4c4ccccc43)cc2)c(-c2c3ccccc3c(-c3ccccc3)c3ccccc23)c1 ; N#Cc1ccc(-c2c3ccccc3c(-c3ccccc3)c3ccccc23)c(-c2ccc(-n3c4ccccc4c4ccccc43)cc2)c1 ; N#Cc1cccc(-c2c3ccccc3c(-c3ccccc3)c3ccccc23)c1-c1ccc(-n2c3ccccc3c3ccccc32)cc1]

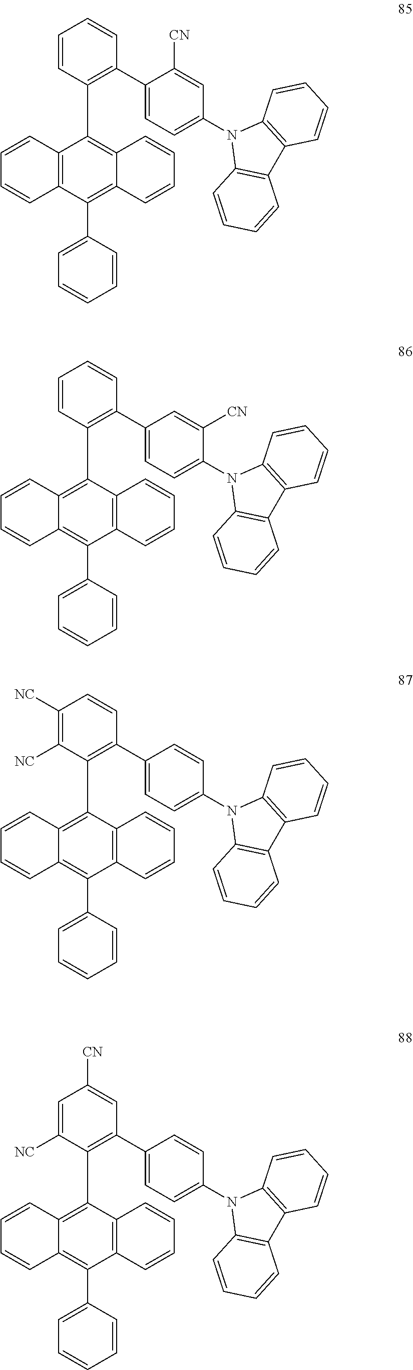 [SMILES: N#Cc1cc(-n2c3ccccc3c3ccccc32)ccc1-c1ccccc1-c1c2ccccc2c(-c2ccccc2)c2ccccc12 ; N#Cc1cc(-c2ccccc2-c2c3ccccc3c(-c3ccccc3)c3ccccc23)ccc1-n1c2ccccc2c2ccccc21 ; N#Cc1ccc(-c2ccc(-n3c4ccccc4c4ccccc43)cc2)c(-c2c3ccccc3c(-c3ccccc3)c3ccccc23)c1C#N ; N#Cc1cc(C#N)c(-c2c3ccccc3c(-c3ccccc3)c3ccccc23)c(-c2ccc(-n3c4ccccc4c4ccccc43)cc2)c1]

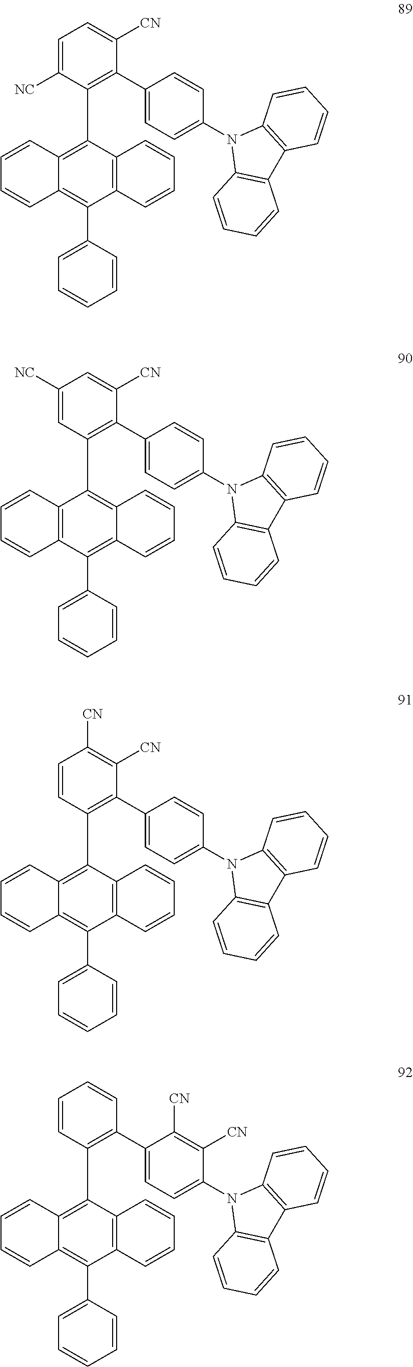 [SMILES: N#Cc1ccc(C#N)c(-c2c3ccccc3c(-c3ccccc3)c3ccccc23)c1-c1ccc(-n2c3ccccc3c3ccccc32)cc1 ; N#Cc1cc(C#N)c(-c2ccc(-n3c4ccccc4c4ccccc43)cc2)c(-c2c3ccccc3c(-c3ccccc3)c3ccccc23)c1 ; N#Cc1ccc(-c2c3ccccc3c(-c3ccccc3)c3ccccc23)c(-c2ccc(-n3c4ccccc4c4ccccc43)cc2)c1C#N ; N#Cc1c(-c2ccccc2-c2c3ccccc3c(-c3ccccc3)c3ccccc23)ccc(-n2c3ccccc3c3ccccc32)c1C#N]

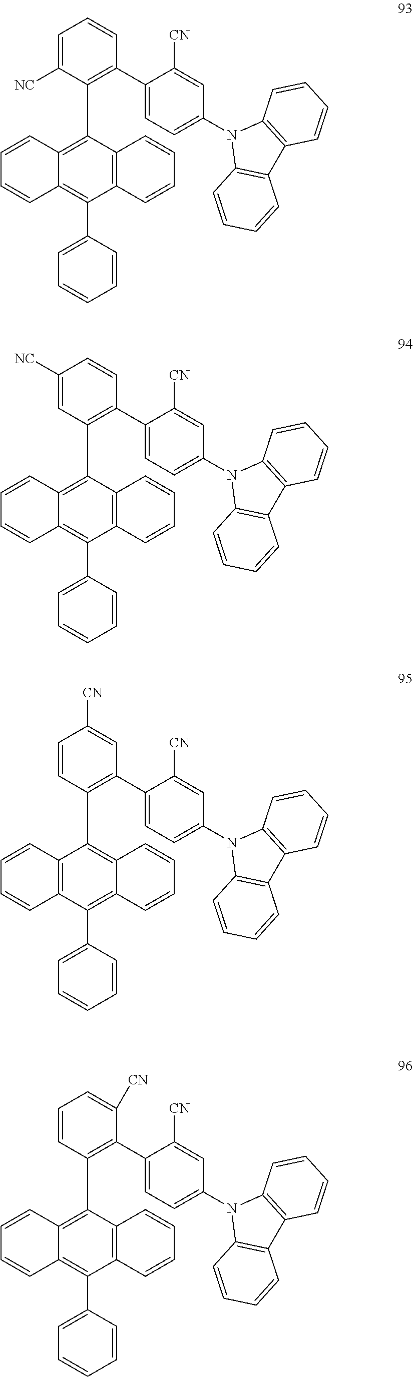 [SMILES: N#Cc1cc(-n2c3ccccc3c3ccccc32)ccc1-c1cccc(C#N)c1-c1c2ccccc2c(-c2ccccc2)c2ccccc12 ; N#Cc1ccc(-c2ccc(-n3c4ccccc4c4ccccc43)cc2C#N)c(-c2c3ccccc3c(-c3ccccc3)c3ccccc23)c1 ; N#Cc1ccc(-c2c3ccccc3c(-c3ccccc3)c3ccccc23)c(-c2ccc(-n3c4ccccc4c4ccccc43)cc2C#N)c1 ; N#Cc1cc(-n2c3ccccc3c3ccccc32)ccc1-c1c(C#N)cccc1-c1c2ccccc2c(-c2ccccc2)c2ccccc12]

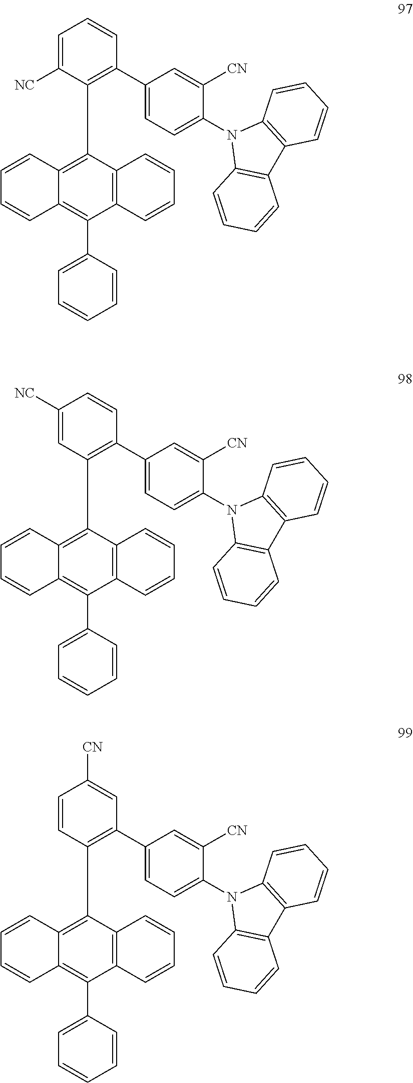 [SMILES: N#Cc1cc(-c2cccc(C#N)c2-c2c3ccccc3c(-c3ccccc3)c3ccccc23)ccc1-n1c2ccccc2c2ccccc21 ; N#Cc1ccc(-c2ccc(-n3c4ccccc4c4ccccc43)c(C#N)c2)c(-c2c3ccccc3c(-c3ccccc3)c3ccccc23)c1 ; N#Cc1ccc(-c2c3ccccc3c(-c3ccccc3)c3ccccc23)c(-c2ccc(-n3c4ccccc4c4ccccc43)c(C#N)c2)c1]

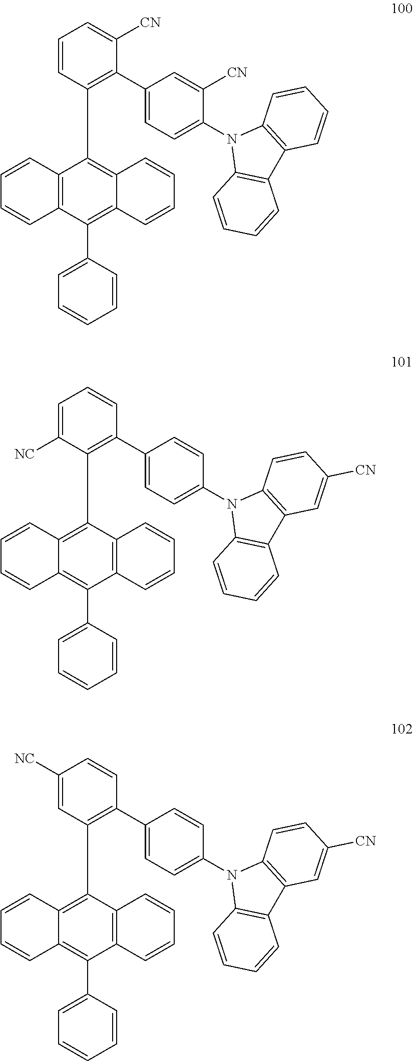 [SMILES: N#Cc1cc(-c2c(C#N)cccc2-c2c3ccccc3c(-c3ccccc3)c3ccccc23)ccc1-n1c2ccccc2c2ccccc21 ; N#Cc1ccc2c(c1)c1ccccc1n2-c1ccc(-c2cccc(C#N)c2-c2c3ccccc3c(-c3ccccc3)c3ccccc23)cc1 ; N#Cc1ccc(-c2ccc(-n3c4ccccc4c4cc(C#N)ccc43)cc2)c(-c2c3ccccc3c(-c3ccccc3)c3ccccc23)c1]

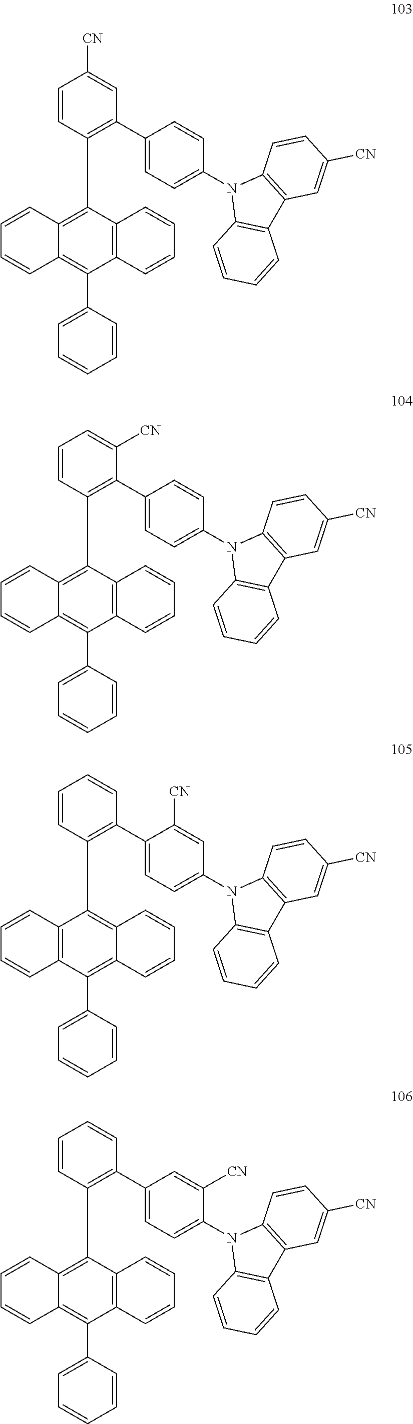

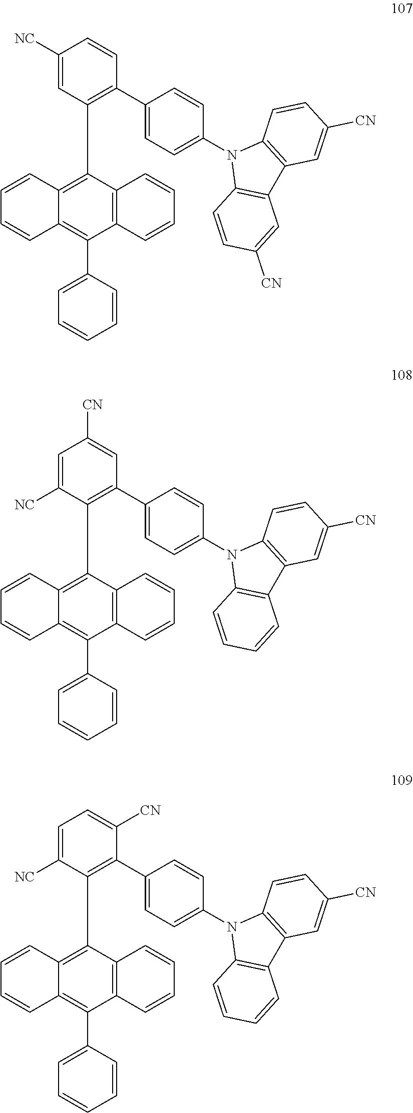 [SMILES: N#Cc1ccc(-c2ccc(-n3c4ccc(C#N)cc4c4cc(C#N)ccc43)cc2)c(-c2c3ccccc3c(-c3ccccc3)c3ccccc23)c1 ; N#Cc1cc(C#N)c(-c2c3ccccc3c(-c3ccccc3)c3ccccc23)c(-c2ccc(-n3c4ccccc4c4cc(C#N)ccc43)cc2)c1 ; N#Cc1ccc2c(c1)c1ccccc1n2-c1ccc(-c2c(C#N)ccc(C#N)c2-c2c3ccccc3c(-c3ccccc3)c3ccccc23)cc1]

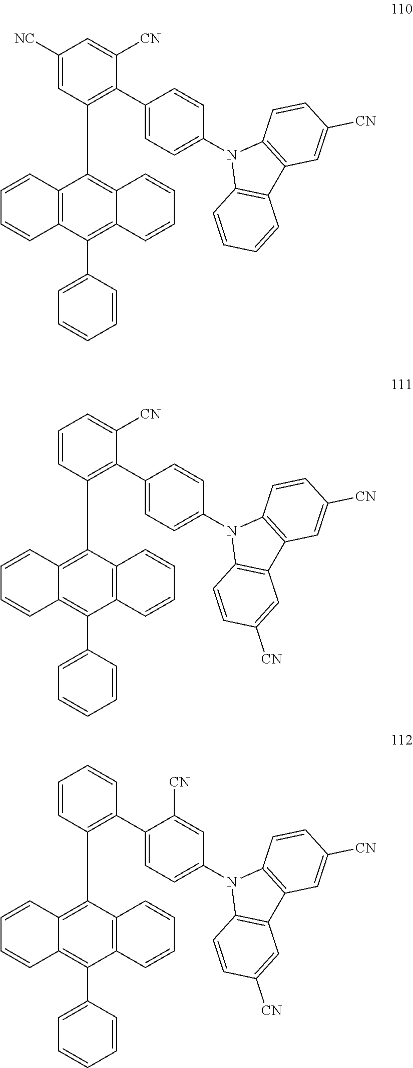 [SMILES: N#Cc1cc(C#N)c(-c2ccc(-n3c4ccccc4c4cc(C#N)ccc43)cc2)c(-c2c3ccccc3c(-c3ccccc3)c3ccccc23)c1 ; N#Cc1ccc2c(c1)c1cc(C#N)ccc1n2-c1ccc(-c2c(C#N)cccc2-c2c3ccccc3c(-c3ccccc3)c3ccccc23)cc1 ; N#Cc1ccc2c(c1)c1cc(C#N)ccc1n2-c1ccc(-c2ccccc2-c2c3ccccc3c(-c3ccccc3)c3ccccc23)c(C#N)c1]

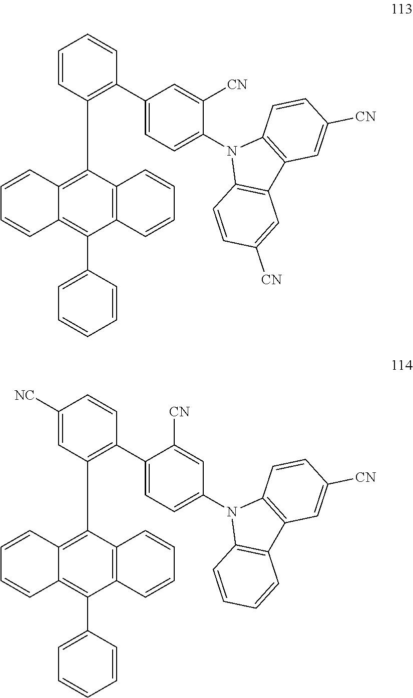 [SMILES: N#Cc1ccc2c(c1)c1cc(C#N)ccc1n2-c1ccc(-c2ccccc2-c2c3ccccc3c(-c3ccccc3)c3ccccc23)cc1C#N ; N#Cc1ccc(-c2ccc(-n3c4ccccc4c4cc(C#N)ccc43)cc2C#N)c(-c2c3ccccc3c(-c3ccccc3)c3ccccc23)c1]

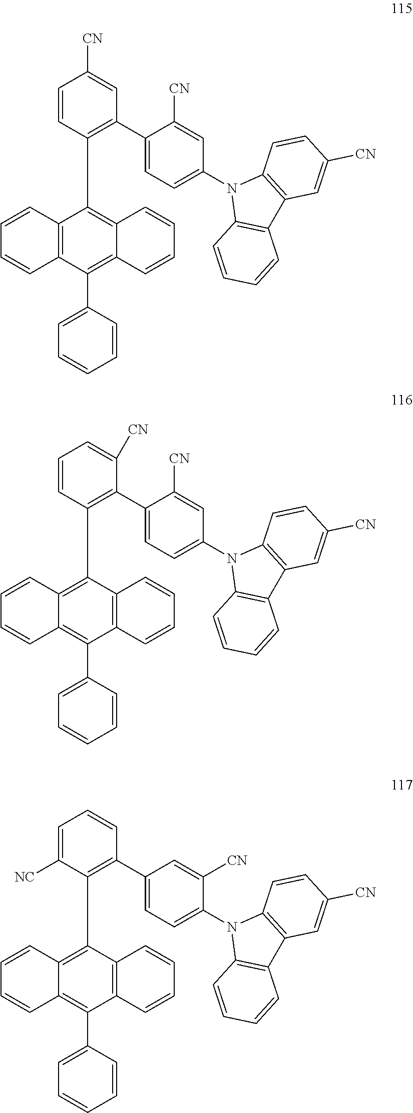 [SMILES: N#Cc1ccc(-c2c3ccccc3c(-c3ccccc3)c3ccccc23)c(-c2ccc(-n3c4ccccc4c4cc(C#N)ccc43)cc2C#N)c1 ; N#Cc1ccc2c(c1)c1ccccc1n2-c1ccc(-c2c(C#N)cccc2-c2c3ccccc3c(-c3ccccc3)c3ccccc23)c(C#N)c1 ; N#Cc1ccc2c(c1)c1ccccc1n2-c1ccc(-c2cccc(C#N)c2-c2c3ccccc3c(-c3ccccc3)c3ccccc23)cc1C#N]

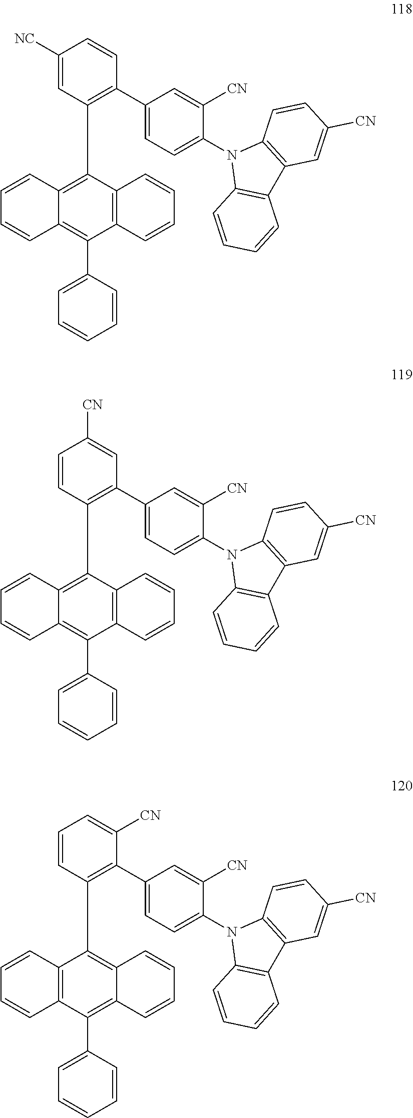 [SMILES: N#Cc1ccc(-c2ccc(-n3c4ccccc4c4cc(C#N)ccc43)c(C#N)c2)c(-c2c3ccccc3c(-c3ccccc3)c3ccccc23)c1 ; N#Cc1ccc(-c2c3ccccc3c(-c3ccccc3)c3ccccc23)c(-c2ccc(-n3c4ccccc4c4cc(C#N)ccc43)c(C#N)c2)c1 ; N#Cc1ccc2c(c1)c1ccccc1n2-c1ccc(-c2c(C#N)cccc2-c2c3ccccc3c(-c3ccccc3)c3ccccc23)cc1C#N]

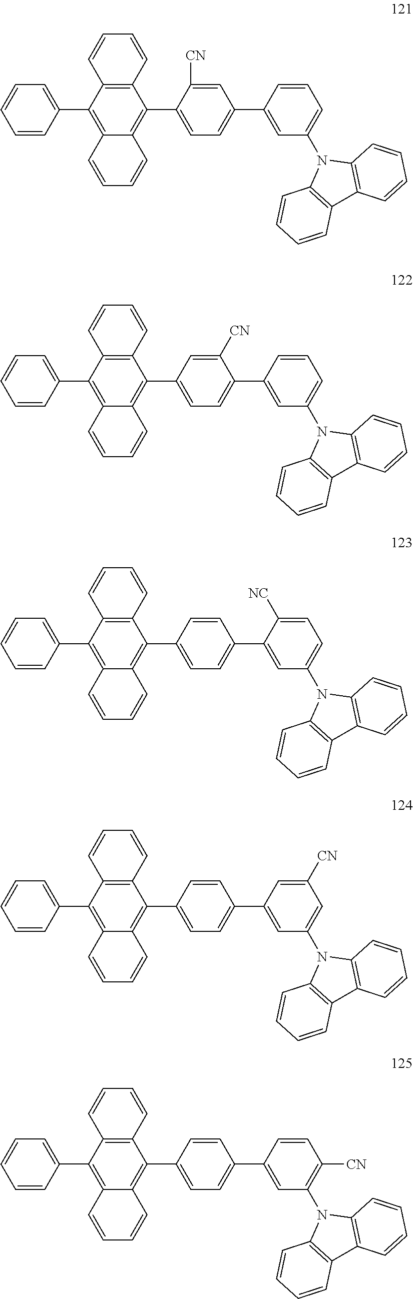 [SMILES: N#Cc1cc(-c2cccc(-n3c4ccccc4c4ccccc43)c2)ccc1-c1c2ccccc2c(-c2ccccc2)c2ccccc12 ; N#Cc1cc(-c2c3ccccc3c(-c3ccccc3)c3ccccc23)ccc1-c1cccc(-n2c3ccccc3c3ccccc32)c1 ; N#Cc1ccc(-n2c3ccccc3c3ccccc32)cc1-c1ccc(-c2c3ccccc3c(-c3ccccc3)c3ccccc23)cc1 ; N#Cc1cc(-c2ccc(-c3c4ccccc4c(-c4ccccc4)c4ccccc34)cc2)cc(-n2c3ccccc3c3ccccc32)c1 ; N#Cc1ccc(-c2ccc(-c3c4ccccc4c(-c4ccccc4)c4ccccc34)cc2)cc1-n1c2ccccc2c2ccccc21]

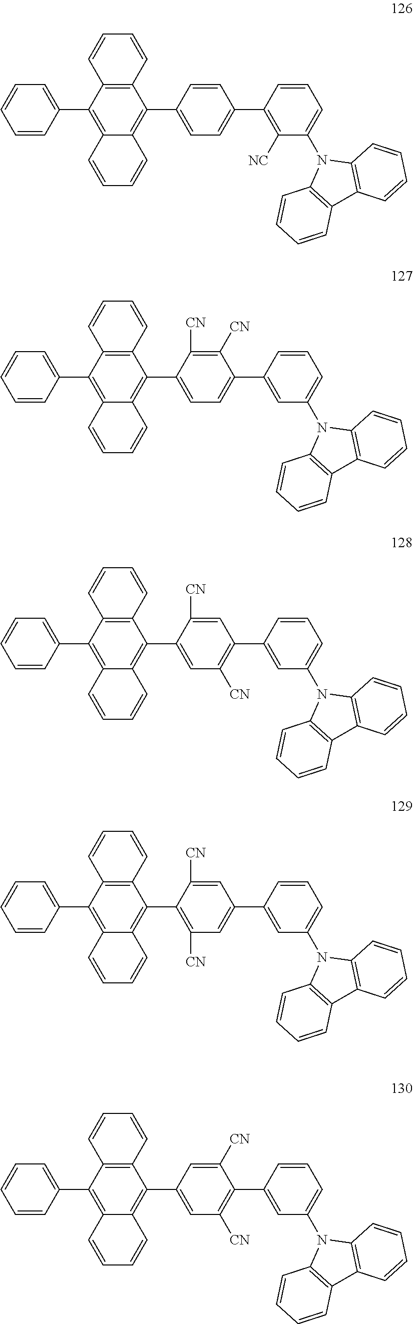 [SMILES: N#Cc1c(-c2ccc(-c3c4ccccc4c(-c4ccccc4)c4ccccc34)cc2)cccc1-n1c2ccccc2c2ccccc21 ; N#Cc1c(-c2cccc(-n3c4ccccc4c4ccccc43)c2)ccc(-c2c3ccccc3c(-c3ccccc3)c3ccccc23)c1C#N ; N#Cc1cc(-c2c3ccccc3c(-c3ccccc3)c3ccccc23)c(C#N)cc1-c1cccc(-n2c3ccccc3c3ccccc32)c1 ; N#Cc1cc(-c2cccc(-n3c4ccccc4c4ccccc43)c2)cc(C#N)c1-c1c2ccccc2c(-c2ccccc2)c2ccccc12 ; N#Cc1cc(-c2c3ccccc3c(-c3ccccc3)c3ccccc23)cc(C#N)c1-c1cccc(-n2c3ccccc3c3ccccc32)c1]

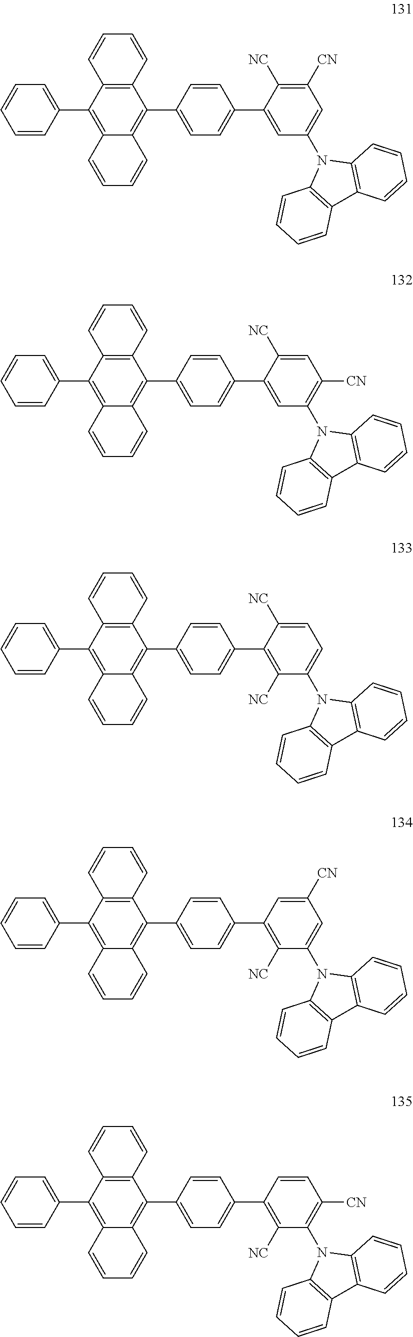 [SMILES: N#Cc1cc(-n2c3ccccc3c3ccccc32)cc(-c2ccc(-c3c4ccccc4c(-c4ccccc4)c4ccccc34)cc2)c1C#N ; N#Cc1cc(C#N)c(-n2c3ccccc3c3ccccc32)cc1-c1ccc(-c2c3ccccc3c(-c3ccccc3)c3ccccc23)cc1 ; N#Cc1ccc(-n2c3ccccc3c3ccccc32)c(C#N)c1-c1ccc(-c2c3ccccc3c(-c3ccccc3)c3ccccc23)cc1 ; N#Cc1cc(-c2ccc(-c3c4ccccc4c(-c4ccccc4)c4ccccc34)cc2)c(C#N)c(-n2c3ccccc3c3ccccc32)c1 ; N#Cc1ccc(-c2ccc(-c3c4ccccc4c(-c4ccccc4)c4ccccc34)cc2)c(C#N)c1-n1c2ccccc2c2ccccc21]

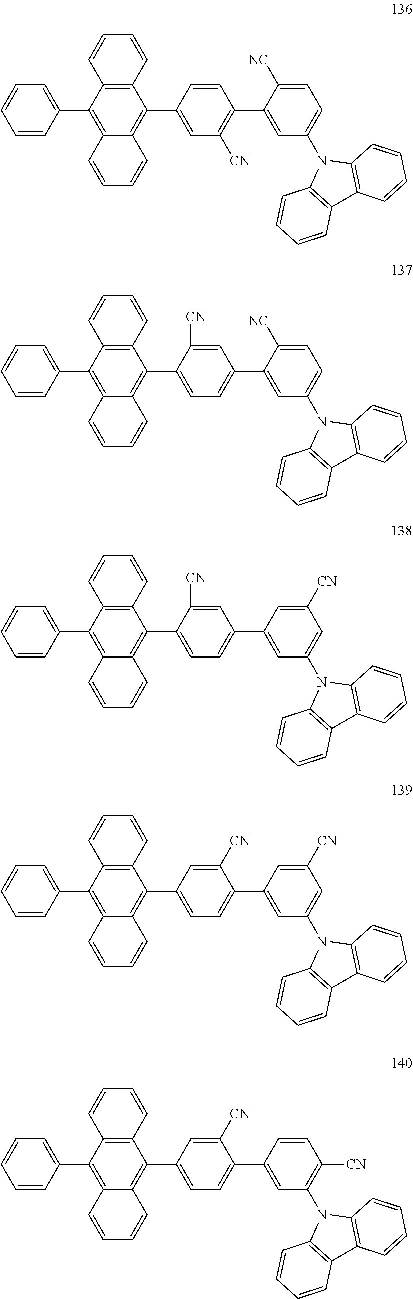 [SMILES: N#Cc1cc(-c2c3ccccc3c(-c3ccccc3)c3ccccc23)ccc1-c1cc(-n2c3ccccc3c3ccccc32)ccc1C#N ; N#Cc1ccc(-n2c3ccccc3c3ccccc32)cc1-c1ccc(-c2c3ccccc3c(-c3ccccc3)c3ccccc23)c(C#N)c1 ; N#Cc1cc(-c2ccc(-c3c4ccccc4c(-c4ccccc4)c4ccccc34)c(C#N)c2)cc(-n2c3ccccc3c3ccccc32)c1 ; N#Cc1cc(-c2ccc(-c3c4ccccc4c(-c4ccccc4)c4ccccc34)cc2C#N)cc(-n2c3ccccc3c3ccccc32)c1 ; N#Cc1cc(-c2c3ccccc3c(-c3ccccc3)c3ccccc23)ccc1-c1ccc(C#N)c(-n2c3ccccc3c3ccccc32)c1]

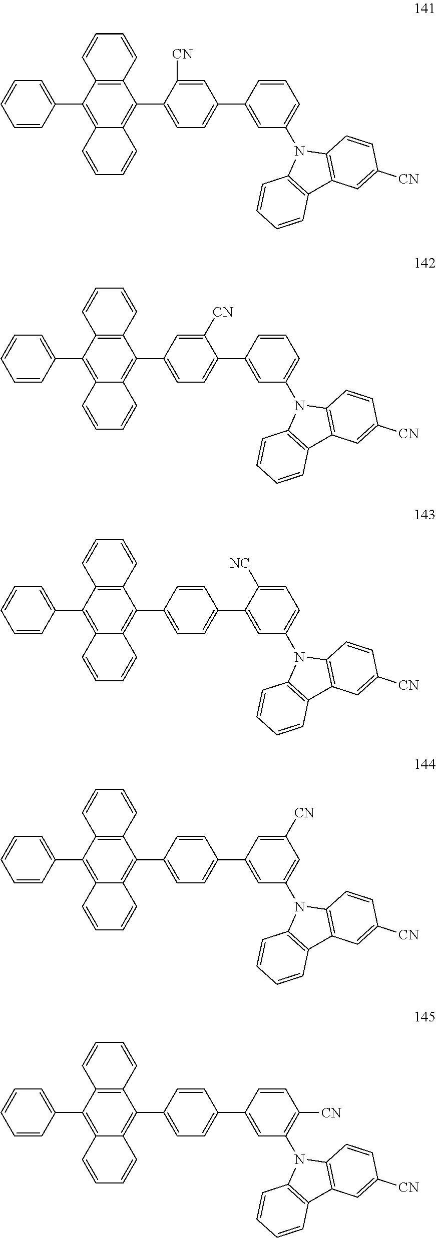 [SMILES: N#Cc1ccc2c(c1)c1ccccc1n2-c1cccc(-c2ccc(-c3c4ccccc4c(-c4ccccc4)c4ccccc34)c(C#N)c2)c1 ; N#Cc1ccc2c(c1)c1ccccc1n2-c1cccc(-c2ccc(-c3c4ccccc4c(-c4ccccc4)c4ccccc34)cc2C#N)c1 ; N#Cc1ccc2c(c1)c1ccccc1n2-c1ccc(C#N)c(-c2ccc(-c3c4ccccc4c(-c4ccccc4)c4ccccc34)cc2)c1 ; N#Cc1cc(-c2ccc(-c3c4ccccc4c(-c4ccccc4)c4ccccc34)cc2)cc(-n2c3ccccc3c3cc(C#N)ccc32)c1 ; N#Cc1ccc2c(c1)c1ccccc1n2-c1cc(-c2ccc(-c3c4ccccc4c(-c4ccccc4)c4ccccc34)cc2)ccc1C#N]

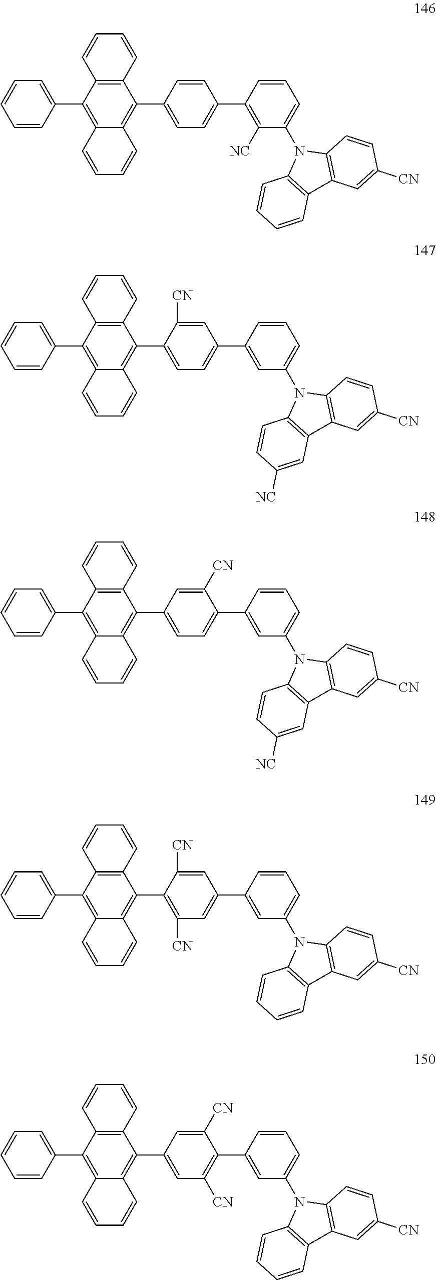 [SMILES: N#Cc1ccc2c(c1)c1ccccc1n2-c1cccc(-c2ccc(-c3c4ccccc4c(-c4ccccc4)c4ccccc34)cc2)c1C#N ; N#Cc1ccc2c(c1)c1cc(C#N)ccc1n2-c1cccc(-c2ccc(-c3c4ccccc4c(-c4ccccc4)c4ccccc34)c(C#N)c2)c1 ; N#Cc1ccc2c(c1)c1cc(C#N)ccc1n2-c1cccc(-c2ccc(-c3c4ccccc4c(-c4ccccc4)c4ccccc34)cc2C#N)c1 ; N#Cc1ccc2c(c1)c1ccccc1n2-c1cccc(-c2cc(C#N)c(-c3c4ccccc4c(-c4ccccc4)c4ccccc34)c(C#N)c2)c1 ; N#Cc1ccc2c(c1)c1ccccc1n2-c1cccc(-c2c(C#N)cc(-c3c4ccccc4c(-c4ccccc4)c4ccccc34)cc2C#N)c1]

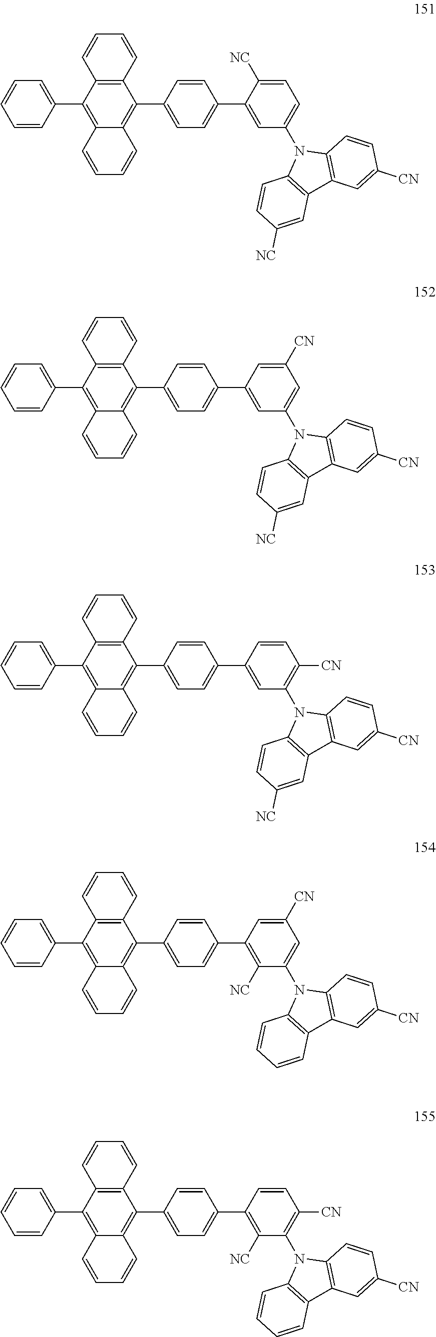 [SMILES: N#Cc1ccc2c(c1)c1cc(C#N)ccc1n2-c1ccc(C#N)c(-c2ccc(-c3c4ccccc4c(-c4ccccc4)c4ccccc34)cc2)c1 ; N#Cc1cc(-c2ccc(-c3c4ccccc4c(-c4ccccc4)c4ccccc34)cc2)cc(-n2c3ccc(C#N)cc3c3cc(C#N)ccc32)c1 ; N#Cc1ccc2c(c1)c1cc(C#N)ccc1n2-c1cc(-c2ccc(-c3c4ccccc4c(-c4ccccc4)c4ccccc34)cc2)ccc1C#N ; N#Cc1cc(-c2ccc(-c3c4ccccc4c(-c4ccccc4)c4ccccc34)cc2)c(C#N)c(-n2c3ccccc3c3cc(C#N)ccc32)c1 ; N#Cc1ccc2c(c1)c1ccccc1n2-c1c(C#N)ccc(-c2ccc(-c3c4ccccc4c(-c4ccccc4)c4ccccc34)cc2)c1C#N]

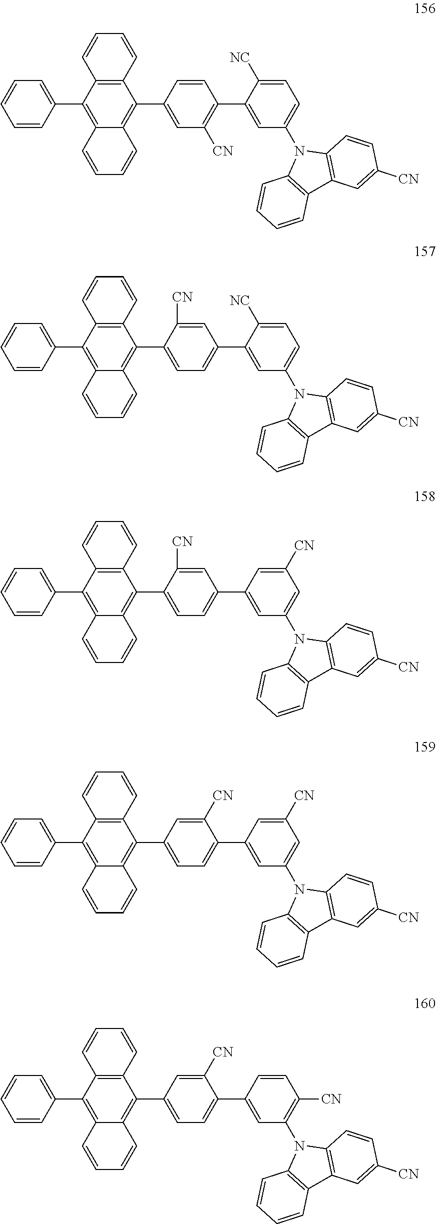 [SMILES: N#Cc1ccc2c(c1)c1ccccc1n2-c1ccc(C#N)c(-c2ccc(-c3c4ccccc4c(-c4ccccc4)c4ccccc34)cc2C#N)c1 ; N#Cc1ccc2c(c1)c1ccccc1n2-c1ccc(C#N)c(-c2ccc(-c3c4ccccc4c(-c4ccccc4)c4ccccc34)c(C#N)c2)c1 ; N#Cc1cc(-c2ccc(-c3c4ccccc4c(-c4ccccc4)c4ccccc34)c(C#N)c2)cc(-n2c3ccccc3c3cc(C#N)ccc32)c1 ; N#Cc1cc(-c2ccc(-c3c4ccccc4c(-c4ccccc4)c4ccccc34)cc2C#N)cc(-n2c3ccccc3c3cc(C#N)ccc32)c1 ; N#Cc1ccc2c(c1)c1ccccc1n2-c1cc(-c2ccc(-c3c4ccccc4c(-c4ccccc4)c4ccccc34)cc2C#N)ccc1C#N]

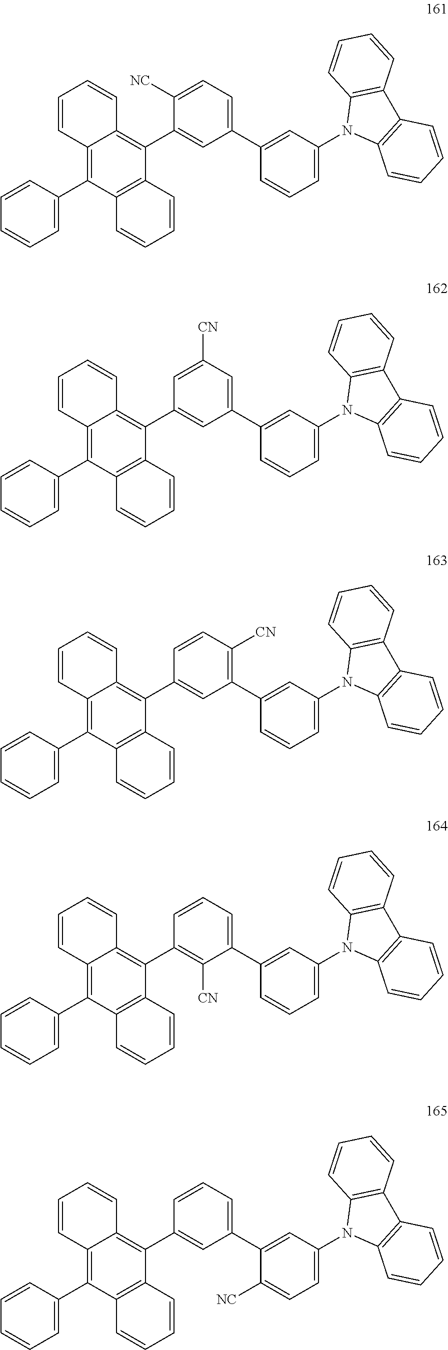 [SMILES: N#Cc1ccc(-c2cccc(-n3c4ccccc4c4ccccc43)c2)cc1-c1c2ccccc2c(-c2ccccc2)c2ccccc12 ; N#Cc1cc(-c2cccc(-n3c4ccccc4c4ccccc43)c2)cc(-c2c3ccccc3c(-c3ccccc3)c3ccccc23)c1 ; N#Cc1ccc(-c2c3ccccc3c(-c3ccccc3)c3ccccc23)cc1-c1cccc(-n2c3ccccc3c3ccccc32)c1 ; N#Cc1c(-c2cccc(-n3c4ccccc4c4ccccc43)c2)cccc1-c1c2ccccc2c(-c2ccccc2)c2ccccc12 ; N#Cc1ccc(-n2c3ccccc3c3ccccc32)cc1-c1cccc(-c2c3ccccc3c(-c3ccccc3)c3ccccc23)c1]

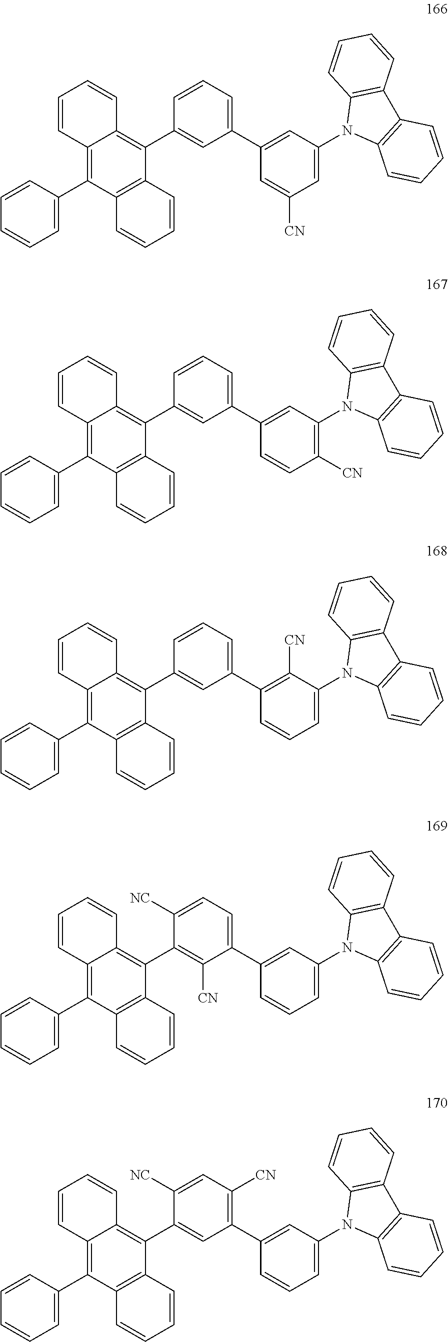 [SMILES: N#Cc1cc(-c2cccc(-c3c4ccccc4c(-c4ccccc4)c4ccccc34)c2)cc(-n2c3ccccc3c3ccccc32)c1 ; N#Cc1ccc(-c2cccc(-c3c4ccccc4c(-c4ccccc4)c4ccccc34)c2)cc1-n1c2ccccc2c2ccccc21 ; N#Cc1c(-c2cccc(-c3c4ccccc4c(-c4ccccc4)c4ccccc34)c2)cccc1-n1c2ccccc2c2ccccc21 ; N#Cc1ccc(-c2cccc(-n3c4ccccc4c4ccccc43)c2)c(C#N)c1-c1c2ccccc2c(-c2ccccc2)c2ccccc12 ; N#Cc1cc(C#N)c(-c2c3ccccc3c(-c3ccccc3)c3ccccc23)cc1-c1cccc(-n2c3ccccc3c3ccccc32)c1]

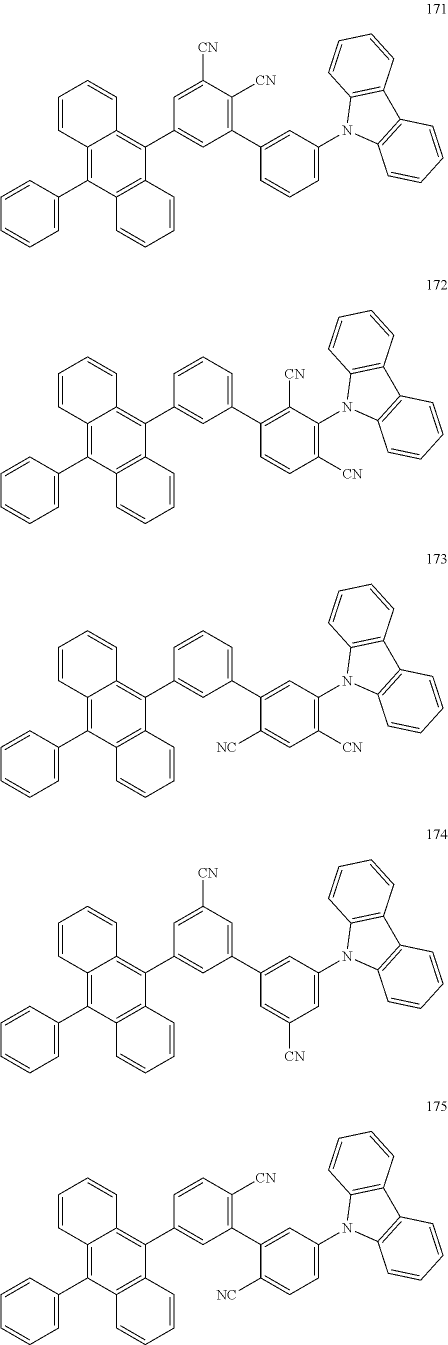 [SMILES: N#Cc1cc(-c2c3ccccc3c(-c3ccccc3)c3ccccc23)cc(-c2cccc(-n3c4ccccc4c4ccccc43)c2)c1C#N ; N#Cc1ccc(-c2cccc(-c3c4ccccc4c(-c4ccccc4)c4ccccc34)c2)c(C#N)c1-n1c2ccccc2c2ccccc21 ; N#Cc1cc(C#N)c(-n2c3ccccc3c3ccccc32)cc1-c1cccc(-c2c3ccccc3c(-c3ccccc3)c3ccccc23)c1 ; N#Cc1cc(-c2cc(C#N)cc(-n3c4ccccc4c4ccccc43)c2)cc(-c2c3ccccc3c(-c3ccccc3)c3ccccc23)c1 ; N#Cc1ccc(-c2c3ccccc3c(-c3ccccc3)c3ccccc23)cc1-c1cc(-n2c3ccccc3c3ccccc32)ccc1C#N]

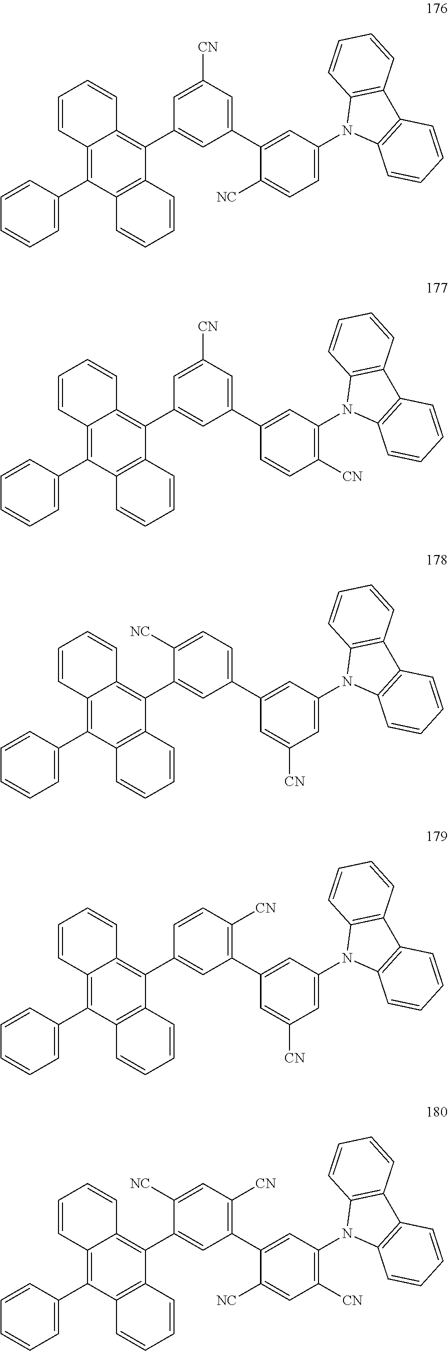 [SMILES: N#Cc1cc(-c2cc(-n3c4ccccc4c4ccccc43)ccc2C#N)cc(-c2c3ccccc3c(-c3ccccc3)c3ccccc23)c1 ; N#Cc1cc(-c2ccc(C#N)c(-n3c4ccccc4c4ccccc43)c2)cc(-c2c3ccccc3c(-c3ccccc3)c3ccccc23)c1 ; N#Cc1cc(-c2ccc(C#N)c(-c3c4ccccc4c(-c4ccccc4)c4ccccc34)c2)cc(-n2c3ccccc3c3ccccc32)c1 ; N#Cc1cc(-c2cc(-c3c4ccccc4c(-c4ccccc4)c4ccccc34)ccc2C#N)cc(-n2c3ccccc3c3ccccc32)c1 ; N#Cc1cc(C#N)c(-c2c3ccccc3c(-c3ccccc3)c3ccccc23)cc1-c1cc(-n2c3ccccc3c3ccccc32)c(C#N)cc1C#N]

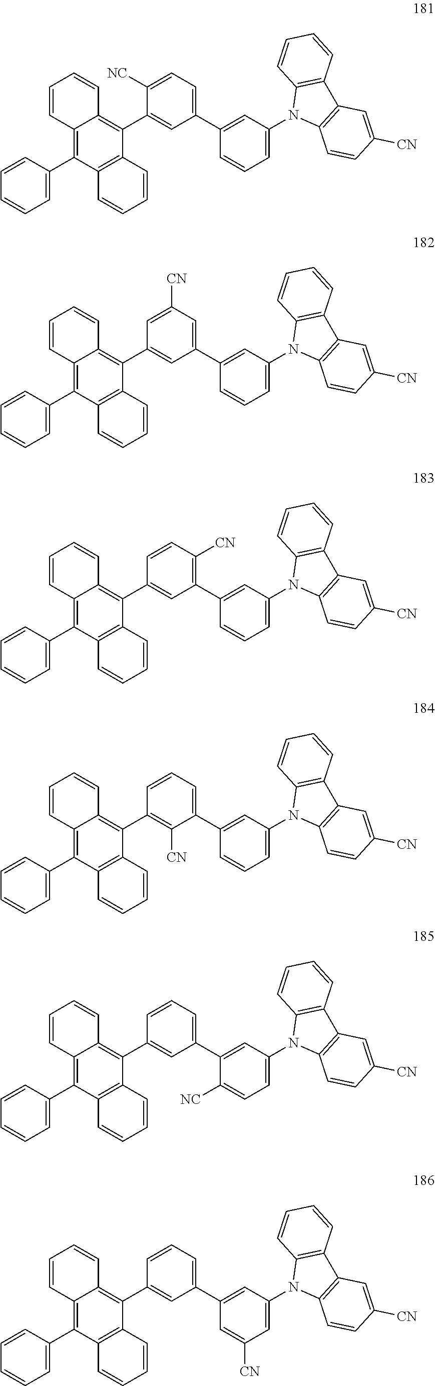

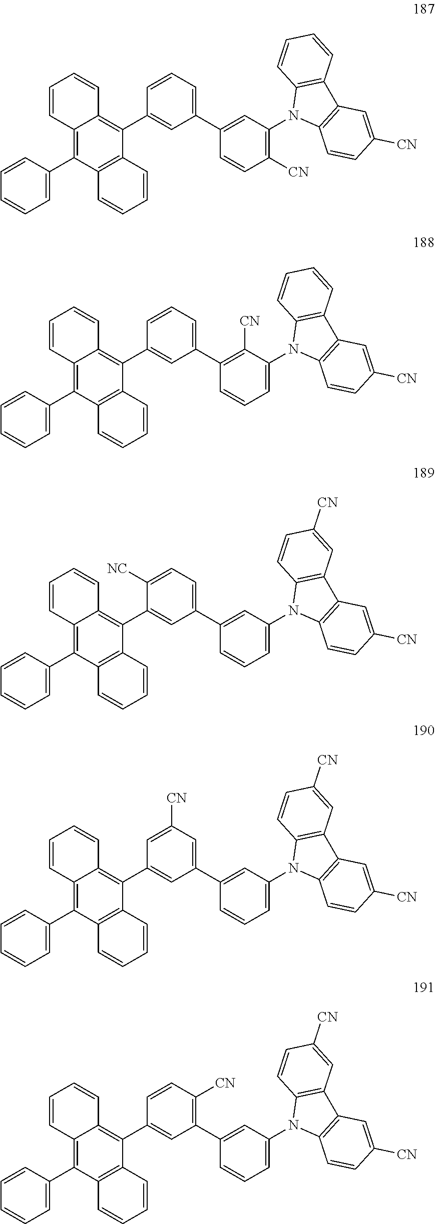 [SMILES: N#Cc1ccc2c(c1)c1ccccc1n2-c1cc(-c2cccc(-c3c4ccccc4c(-c4ccccc4)c4ccccc34)c2)ccc1C#N ; N#Cc1ccc2c(c1)c1ccccc1n2-c1cccc(-c2cccc(-c3c4ccccc4c(-c4ccccc4)c4ccccc34)c2)c1C#N ; N#Cc1ccc2c(c1)c1cc(C#N)ccc1n2-c1cccc(-c2ccc(C#N)c(-c3c4ccccc4c(-c4ccccc4)c4ccccc34)c2)c1 ; N#Cc1cc(-c2cccc(-n3c4ccc(C#N)cc4c4cc(C#N)ccc43)c2)cc(-c2c3ccccc3c(-c3ccccc3)c3ccccc23)c1 ; N#Cc1ccc2c(c1)c1cc(C#N)ccc1n2-c1cccc(-c2cc(-c3c4ccccc4c(-c4ccccc4)c4ccccc34)ccc2C#N)c1]

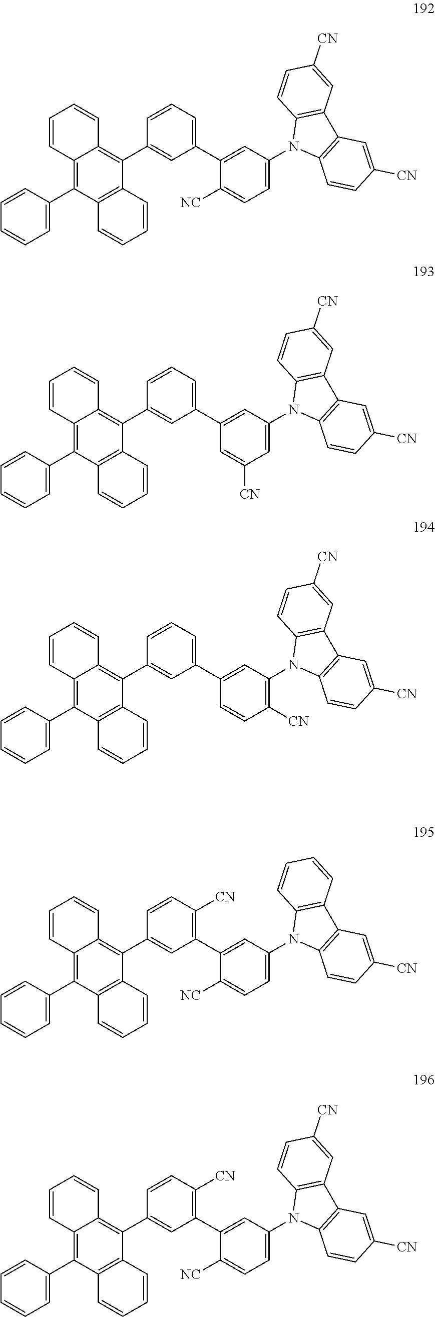 [SMILES: N#Cc1ccc2c(c1)c1cc(C#N)ccc1n2-c1ccc(C#N)c(-c2cccc(-c3c4ccccc4c(-c4ccccc4)c4ccccc34)c2)c1 ; N#Cc1cc(-c2cccc(-c3c4ccccc4c(-c4ccccc4)c4ccccc34)c2)cc(-n2c3ccc(C#N)cc3c3cc(C#N)ccc32)c1 ; N#Cc1ccc2c(c1)c1cc(C#N)ccc1n2-c1cc(-c2cccc(-c3c4ccccc4c(-c4ccccc4)c4ccccc34)c2)ccc1C#N ; N#Cc1ccc2c(c1)c1ccccc1n2-c1ccc(C#N)c(-c2cc(-c3c4ccccc4c(-c4ccccc4)c4ccccc34)ccc2C#N)c1 ; N#Cc1ccc2c(c1)c1cc(C#N)ccc1n2-c1ccc(C#N)c(-c2cc(-c3c4ccccc4c(-c4ccccc4)c4ccccc34)ccc2C#N)c1]

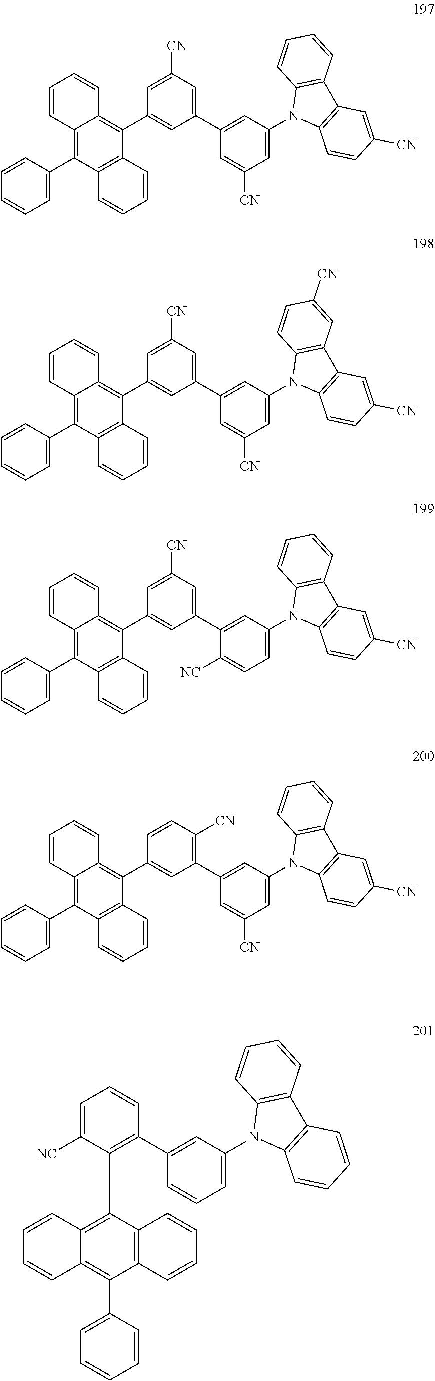 [SMILES: N#Cc1cc(-c2cc(C#N)cc(-n3c4ccccc4c4cc(C#N)ccc43)c2)cc(-c2c3ccccc3c(-c3ccccc3)c3ccccc23)c1 ; N#Cc1cc(-c2cc(C#N)cc(-n3c4ccc(C#N)cc4c4cc(C#N)ccc43)c2)cc(-c2c3ccccc3c(-c3ccccc3)c3ccccc23)c1 ; N#Cc1cc(-c2cc(-n3c4ccccc4c4cc(C#N)ccc43)ccc2C#N)cc(-c2c3ccccc3c(-c3ccccc3)c3ccccc23)c1 ; N#Cc1cc(-c2cc(-c3c4ccccc4c(-c4ccccc4)c4ccccc34)ccc2C#N)cc(-n2c3ccccc3c3cc(C#N)ccc32)c1 ; N#Cc1cccc(-c2cccc(-n3c4ccccc4c4ccccc43)c2)c1-c1c2ccccc2c(-c2ccccc2)c2ccccc12]

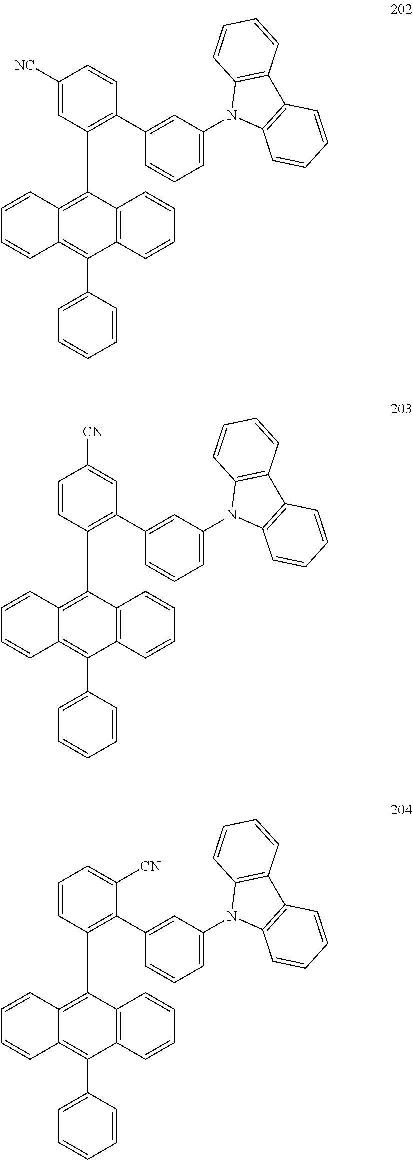 [SMILES: N#Cc1ccc(-c2cccc(-n3c4ccccc4c4ccccc43)c2)c(-c2c3ccccc3c(-c3ccccc3)c3ccccc23)c1 ; N#Cc1ccc(-c2c3ccccc3c(-c3ccccc3)c3ccccc23)c(-c2cccc(-n3c4ccccc4c4ccccc43)c2)c1 ; N#Cc1cccc(-c2c3ccccc3c(-c3ccccc3)c3ccccc23)c1-c1cccc(-n2c3ccccc3c3ccccc32)c1]

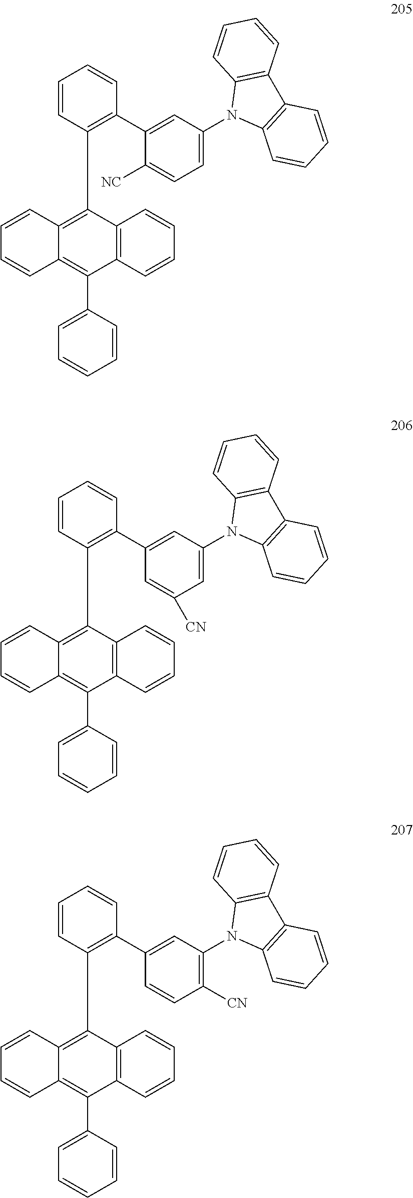 [SMILES: N#Cc1ccc(-n2c3ccccc3c3ccccc32)cc1-c1ccccc1-c1c2ccccc2c(-c2ccccc2)c2ccccc12 ; N#Cc1cc(-c2ccccc2-c2c3ccccc3c(-c3ccccc3)c3ccccc23)cc(-n2c3ccccc3c3ccccc32)c1 ; N#Cc1ccc(-c2ccccc2-c2c3ccccc3c(-c3ccccc3)c3ccccc23)cc1-n1c2ccccc2c2ccccc21]

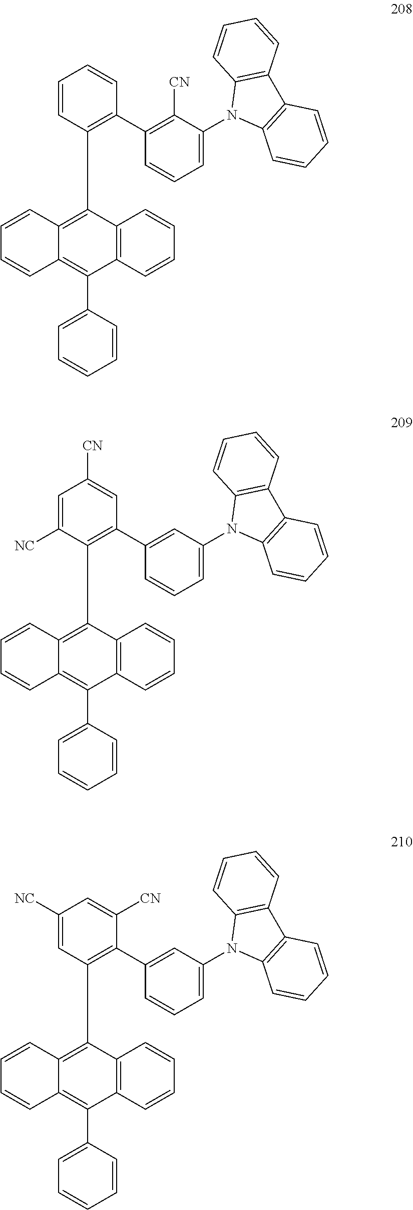 [SMILES: N#Cc1c(-c2ccccc2-c2c3ccccc3c(-c3ccccc3)c3ccccc23)cccc1-n1c2ccccc2c2ccccc21 ; N#Cc1cc(C#N)c(-c2c3ccccc3c(-c3ccccc3)c3ccccc23)c(-c2cccc(-n3c4ccccc4c4ccccc43)c2)c1 ; N#Cc1cc(C#N)c(-c2cccc(-n3c4ccccc4c4ccccc43)c2)c(-c2c3ccccc3c(-c3ccccc3)c3ccccc23)c1]

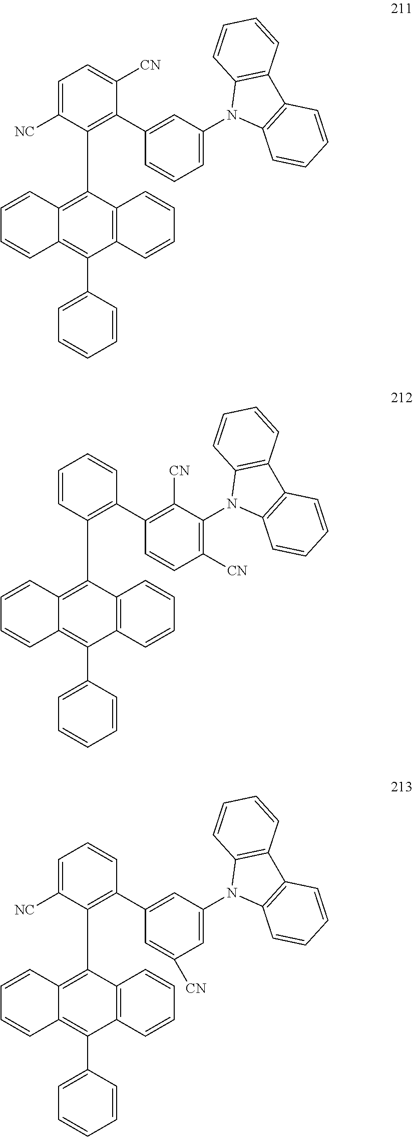 [SMILES: N#Cc1ccc(C#N)c(-c2c3ccccc3c(-c3ccccc3)c3ccccc23)c1-c1cccc(-n2c3ccccc3c3ccccc32)c1 ; N#Cc1ccc(-c2ccccc2-c2c3ccccc3c(-c3ccccc3)c3ccccc23)c(C#N)c1-n1c2ccccc2c2ccccc21 ; N#Cc1cc(-c2cccc(C#N)c2-c2c3ccccc3c(-c3ccccc3)c3ccccc23)cc(-n2c3ccccc3c3ccccc32)c1]

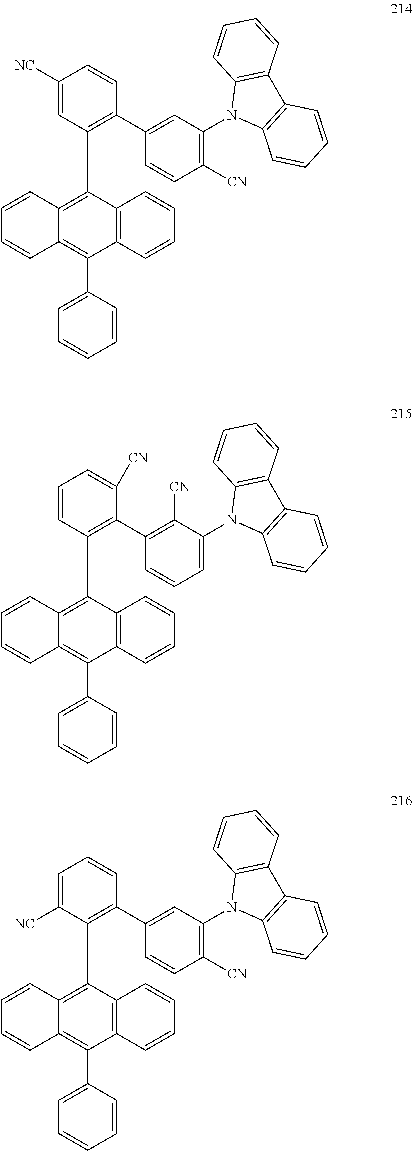 [SMILES: N#Cc1ccc(-c2ccc(C#N)c(-n3c4ccccc4c4ccccc43)c2)c(-c2c3ccccc3c(-c3ccccc3)c3ccccc23)c1 ; N#Cc1cccc(-c2c3ccccc3c(-c3ccccc3)c3ccccc23)c1-c1cccc(-n2c3ccccc3c3ccccc32)c1C#N ; N#Cc1ccc(-c2cccc(C#N)c2-c2c3ccccc3c(-c3ccccc3)c3ccccc23)cc1-n1c2ccccc2c2ccccc21]

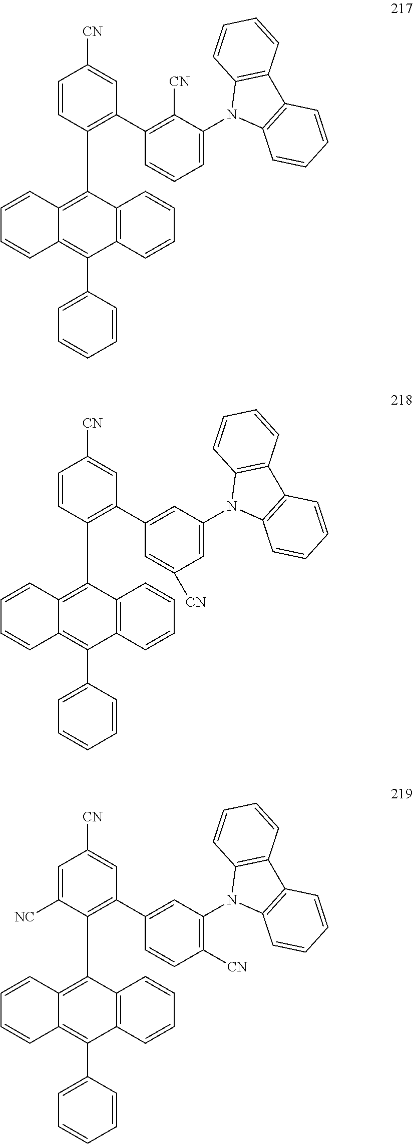 [SMILES: N#Cc1ccc(-c2c3ccccc3c(-c3ccccc3)c3ccccc23)c(-c2cccc(-n3c4ccccc4c4ccccc43)c2C#N)c1 ; N#Cc1cc(-c2cc(C#N)ccc2-c2c3ccccc3c(-c3ccccc3)c3ccccc23)cc(-n2c3ccccc3c3ccccc32)c1 ; N#Cc1cc(C#N)c(-c2c3ccccc3c(-c3ccccc3)c3ccccc23)c(-c2ccc(C#N)c(-n3c4ccccc4c4ccccc43)c2)c1]

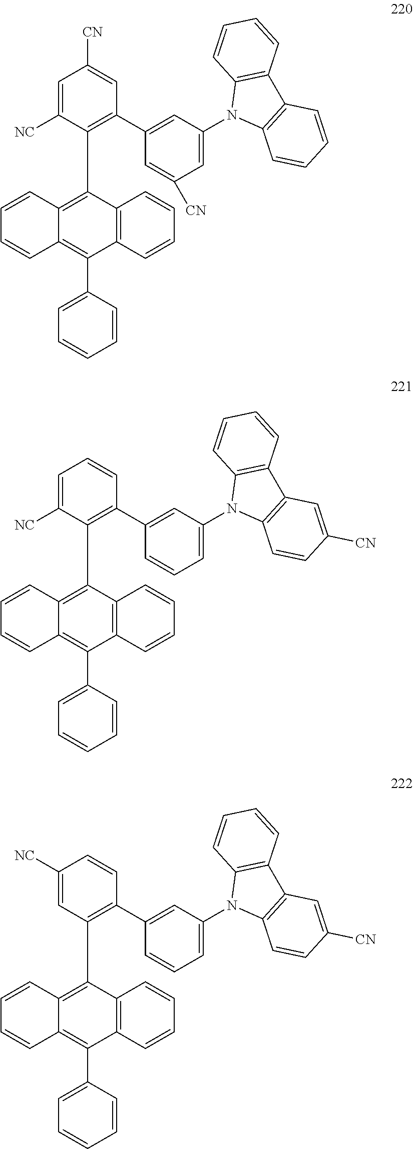 [SMILES: N#Cc1cc(-c2cc(C#N)cc(C#N)c2-c2c3ccccc3c(-c3ccccc3)c3ccccc23)cc(-n2c3ccccc3c3ccccc32)c1 ; N#Cc1ccc2c(c1)c1ccccc1n2-c1cccc(-c2cccc(C#N)c2-c2c3ccccc3c(-c3ccccc3)c3ccccc23)c1 ; N#Cc1ccc(-c2cccc(-n3c4ccccc4c4cc(C#N)ccc43)c2)c(-c2c3ccccc3c(-c3ccccc3)c3ccccc23)c1]

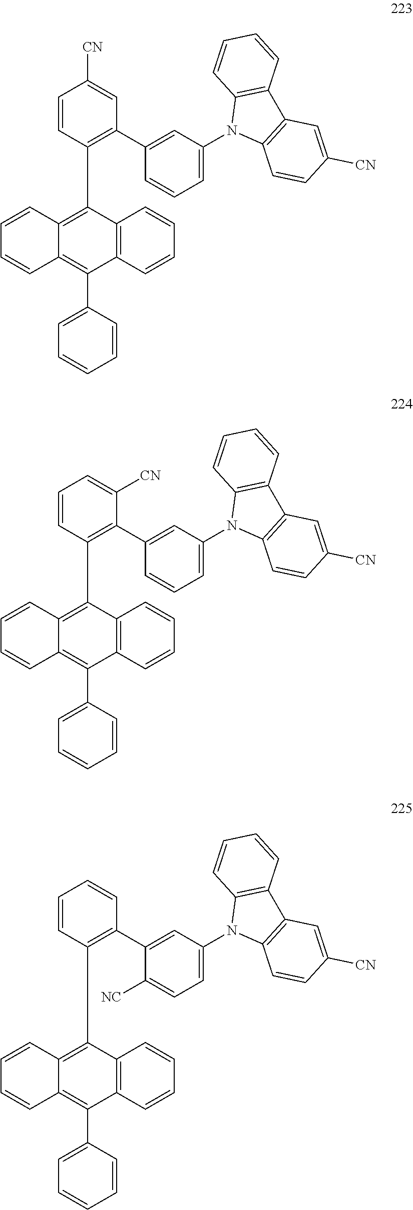 [SMILES: N#Cc1ccc(-c2c3ccccc3c(-c3ccccc3)c3ccccc23)c(-c2cccc(-n3c4ccccc4c4cc(C#N)ccc43)c2)c1 ; N#Cc1ccc2c(c1)c1ccccc1n2-c1cccc(-c2c(C#N)cccc2-c2c3ccccc3c(-c3ccccc3)c3ccccc23)c1 ; N#Cc1ccc2c(c1)c1ccccc1n2-c1ccc(C#N)c(-c2ccccc2-c2c3ccccc3c(-c3ccccc3)c3ccccc23)c1]

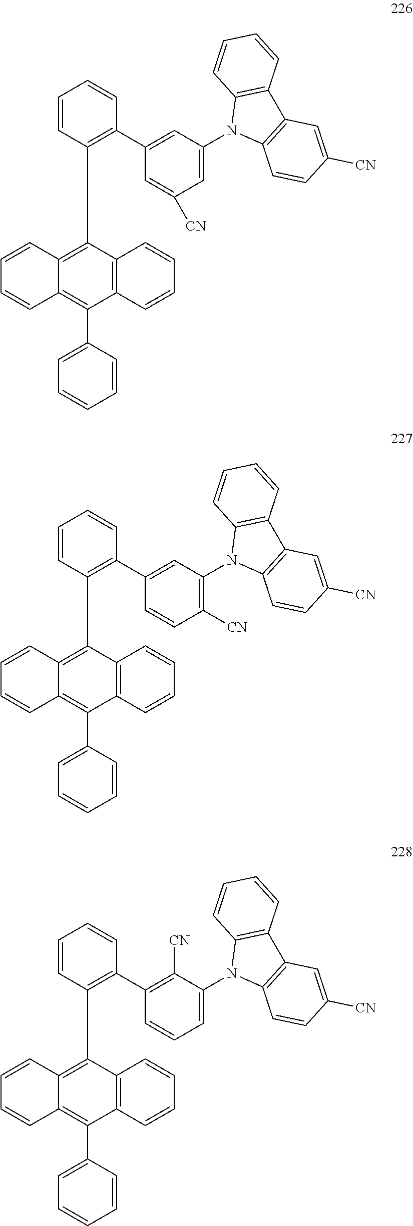 [SMILES: N#Cc1cc(-c2ccccc2-c2c3ccccc3c(-c3ccccc3)c3ccccc23)cc(-n2c3ccccc3c3cc(C#N)ccc32)c1 ; N#Cc1ccc2c(c1)c1ccccc1n2-c1cc(-c2ccccc2-c2c3ccccc3c(-c3ccccc3)c3ccccc23)ccc1C#N ; N#Cc1ccc2c(c1)c1ccccc1n2-c1cccc(-c2ccccc2-c2c3ccccc3c(-c3ccccc3)c3ccccc23)c1C#N]

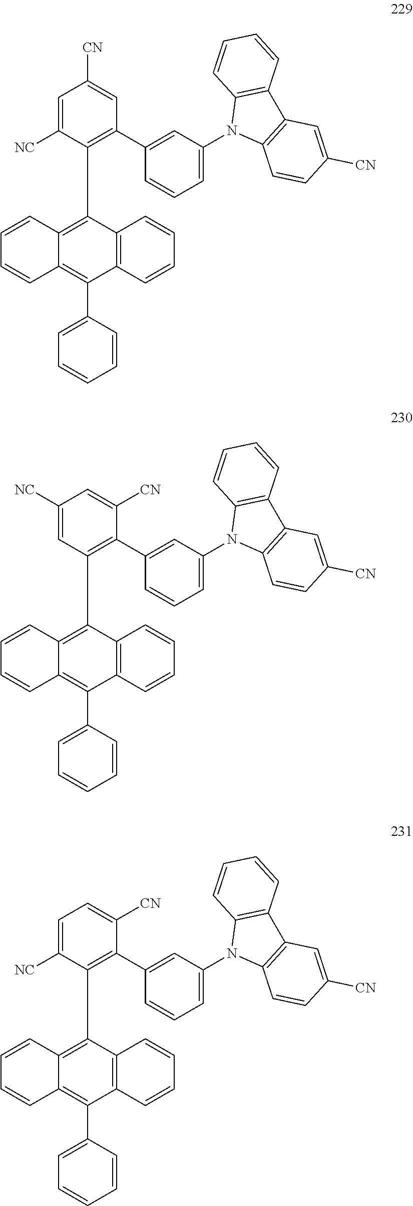 [SMILES: N#Cc1cc(C#N)c(-c2c3ccccc3c(-c3ccccc3)c3ccccc23)c(-c2cccc(-n3c4ccccc4c4cc(C#N)ccc43)c2)c1 ; N#Cc1cc(C#N)c(-c2cccc(-n3c4ccccc4c4cc(C#N)ccc43)c2)c(-c2c3ccccc3c(-c3ccccc3)c3ccccc23)c1 ; N#Cc1ccc2c(c1)c1ccccc1n2-c1cccc(-c2c(C#N)ccc(C#N)c2-c2c3ccccc3c(-c3ccccc3)c3ccccc23)c1]

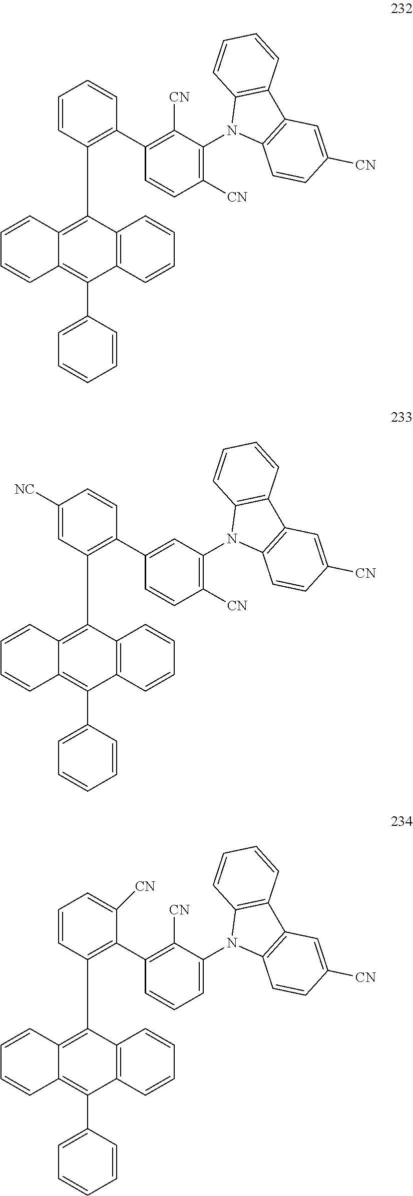 [SMILES: N#Cc1ccc2c(c1)c1ccccc1n2-c1c(C#N)ccc(-c2ccccc2-c2c3ccccc3c(-c3ccccc3)c3ccccc23)c1C#N ; N#Cc1ccc(-c2ccc(C#N)c(-n3c4ccccc4c4cc(C#N)ccc43)c2)c(-c2c3ccccc3c(-c3ccccc3)c3ccccc23)c1 ; N#Cc1ccc2c(c1)c1ccccc1n2-c1cccc(-c2c(C#N)cccc2-c2c3ccccc3c(-c3ccccc3)c3ccccc23)c1C#N]

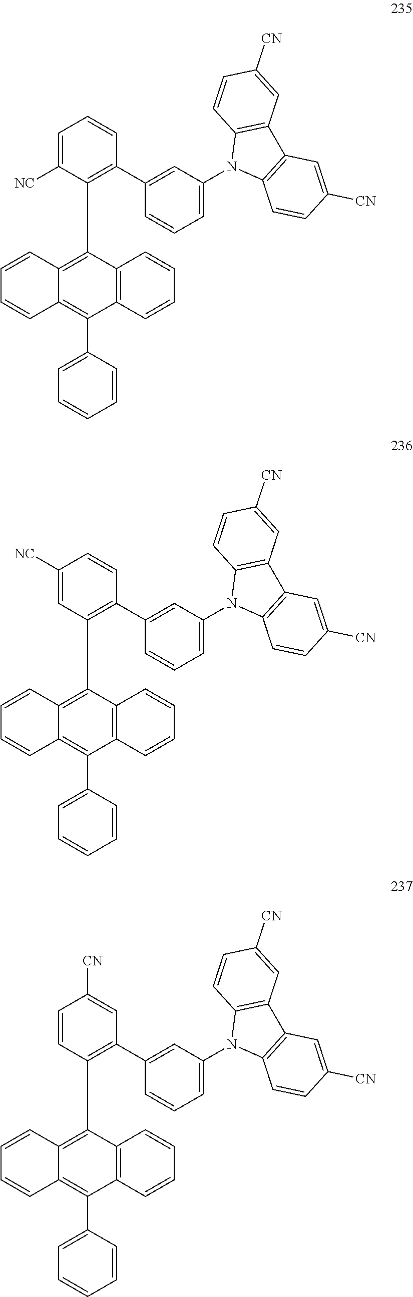 [SMILES: N#Cc1ccc2c(c1)c1cc(C#N)ccc1n2-c1cccc(-c2cccc(C#N)c2-c2c3ccccc3c(-c3ccccc3)c3ccccc23)c1 ; N#Cc1ccc(-c2cccc(-n3c4ccc(C#N)cc4c4cc(C#N)ccc43)c2)c(-c2c3ccccc3c(-c3ccccc3)c3ccccc23)c1 ; N#Cc1ccc(-c2c3ccccc3c(-c3ccccc3)c3ccccc23)c(-c2cccc(-n3c4ccc(C#N)cc4c4cc(C#N)ccc43)c2)c1]

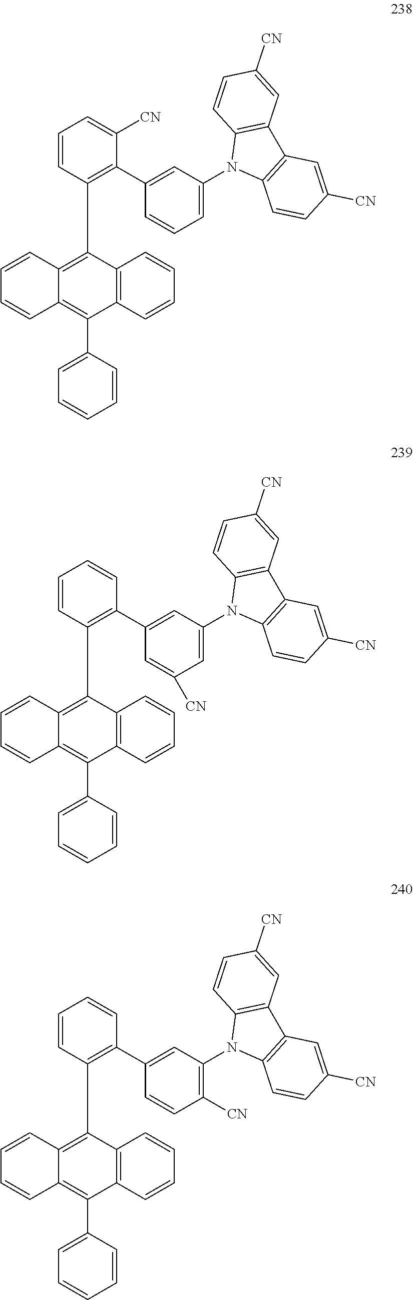 [SMILES: N#Cc1ccc2c(c1)c1cc(C#N)ccc1n2-c1cccc(-c2c(C#N)cccc2-c2c3ccccc3c(-c3ccccc3)c3ccccc23)c1 ; N#Cc1cc(-c2ccccc2-c2c3ccccc3c(-c3ccccc3)c3ccccc23)cc(-n2c3ccc(C#N)cc3c3cc(C#N)ccc32)c1 ; N#Cc1ccc2c(c1)c1cc(C#N)ccc1n2-c1cc(-c2ccccc2-c2c3ccccc3c(-c3ccccc3)c3ccccc23)ccc1C#N]

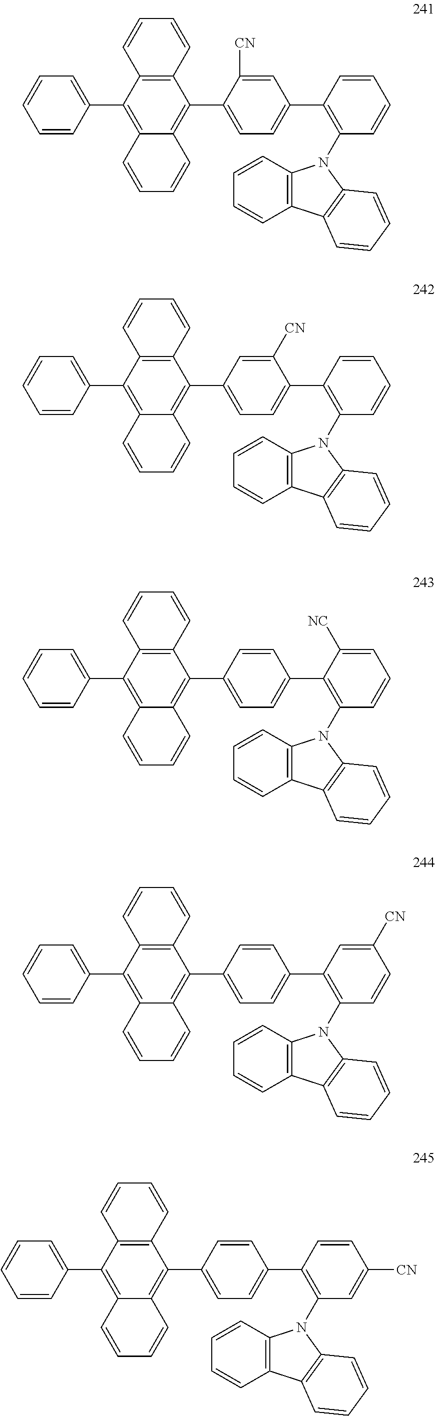 [SMILES: N#Cc1cc(-c2ccccc2-n2c3ccccc3c3ccccc32)ccc1-c1c2ccccc2c(-c2ccccc2)c2ccccc12 ; N#Cc1cc(-c2c3ccccc3c(-c3ccccc3)c3ccccc23)ccc1-c1ccccc1-n1c2ccccc2c2ccccc21 ; N#Cc1cccc(-n2c3ccccc3c3ccccc32)c1-c1ccc(-c2c3ccccc3c(-c3ccccc3)c3ccccc23)cc1 ; N#Cc1ccc(-n2c3ccccc3c3ccccc32)c(-c2ccc(-c3c4ccccc4c(-c4ccccc4)c4ccccc34)cc2)c1 ; N#Cc1ccc(-c2ccc(-c3c4ccccc4c(-c4ccccc4)c4ccccc34)cc2)c(-n2c3ccccc3c3ccccc32)c1]

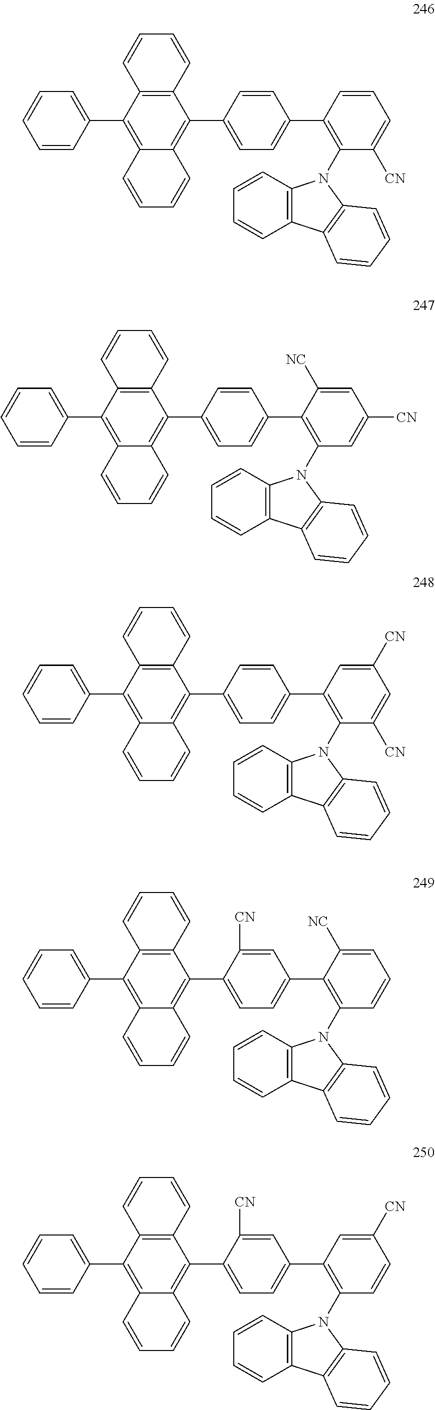 [SMILES: N#Cc1cccc(-c2ccc(-c3c4ccccc4c(-c4ccccc4)c4ccccc34)cc2)c1-n1c2ccccc2c2ccccc21 ; N#Cc1cc(C#N)c(-c2ccc(-c3c4ccccc4c(-c4ccccc4)c4ccccc34)cc2)c(-n2c3ccccc3c3ccccc32)c1 ; N#Cc1cc(C#N)c(-n2c3ccccc3c3ccccc32)c(-c2ccc(-c3c4ccccc4c(-c4ccccc4)c4ccccc34)cc2)c1 ; N#Cc1cc(-c2c(C#N)cccc2-n2c3ccccc3c3ccccc32)ccc1-c1c2ccccc2c(-c2ccccc2)c2ccccc12 ; N#Cc1ccc(-n2c3ccccc3c3ccccc32)c(-c2ccc(-c3c4ccccc4c(-c4ccccc4)c4ccccc34)c(C#N)c2)c1]

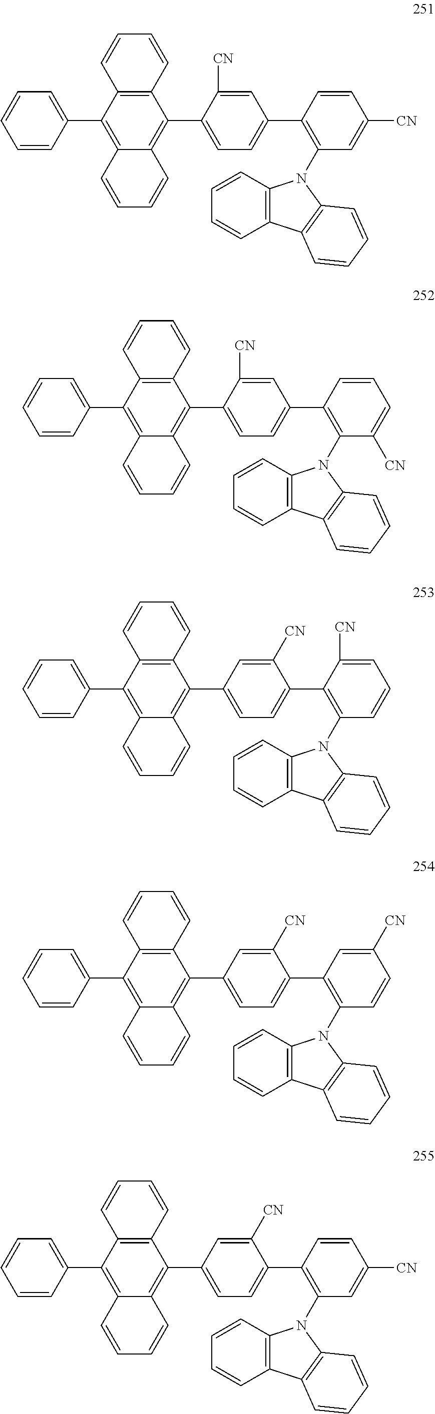 [SMILES: N#Cc1ccc(-c2ccc(-c3c4ccccc4c(-c4ccccc4)c4ccccc34)c(C#N)c2)c(-n2c3ccccc3c3ccccc32)c1 ; N#Cc1cc(-c2cccc(C#N)c2-n2c3ccccc3c3ccccc32)ccc1-c1c2ccccc2c(-c2ccccc2)c2ccccc12 ; N#Cc1cc(-c2c3ccccc3c(-c3ccccc3)c3ccccc23)ccc1-c1c(C#N)cccc1-n1c2ccccc2c2ccccc21 ; N#Cc1ccc(-n2c3ccccc3c3ccccc32)c(-c2ccc(-c3c4ccccc4c(-c4ccccc4)c4ccccc34)cc2C#N)c1 ; N#Cc1ccc(-c2ccc(-c3c4ccccc4c(-c4ccccc4)c4ccccc34)cc2C#N)c(-n2c3ccccc3c3ccccc32)c1]

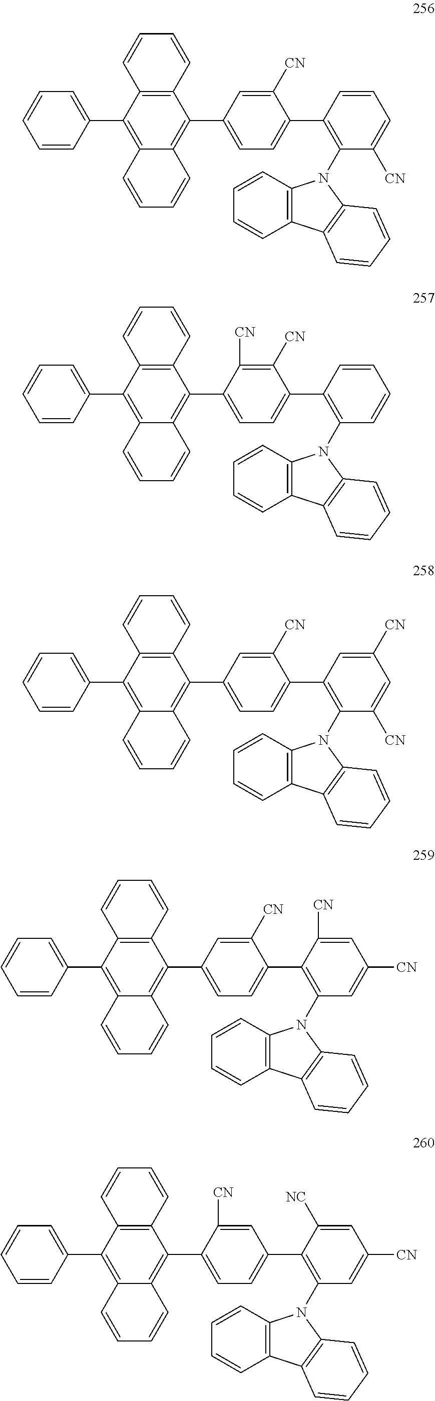 [SMILES: N#Cc1cc(-c2c3ccccc3c(-c3ccccc3)c3ccccc23)ccc1-c1cccc(C#N)c1-n1c2ccccc2c2ccccc21 ; N#Cc1c(-c2ccccc2-n2c3ccccc3c3ccccc32)ccc(-c2c3ccccc3c(-c3ccccc3)c3ccccc23)c1C#N ; N#Cc1cc(C#N)c(-n2c3ccccc3c3ccccc32)c(-c2ccc(-c3c4ccccc4c(-c4ccccc4)c4ccccc34)cc2C#N)c1 ; N#Cc1cc(C#N)c(-c2ccc(-c3c4ccccc4c(-c4ccccc4)c4ccccc34)cc2C#N)c(-n2c3ccccc3c3ccccc32)c1 ; N#Cc1cc(C#N)c(-c2ccc(-c3c4ccccc4c(-c4ccccc4)c4ccccc34)c(C#N)c2)c(-n2c3ccccc3c3ccccc32)c1]

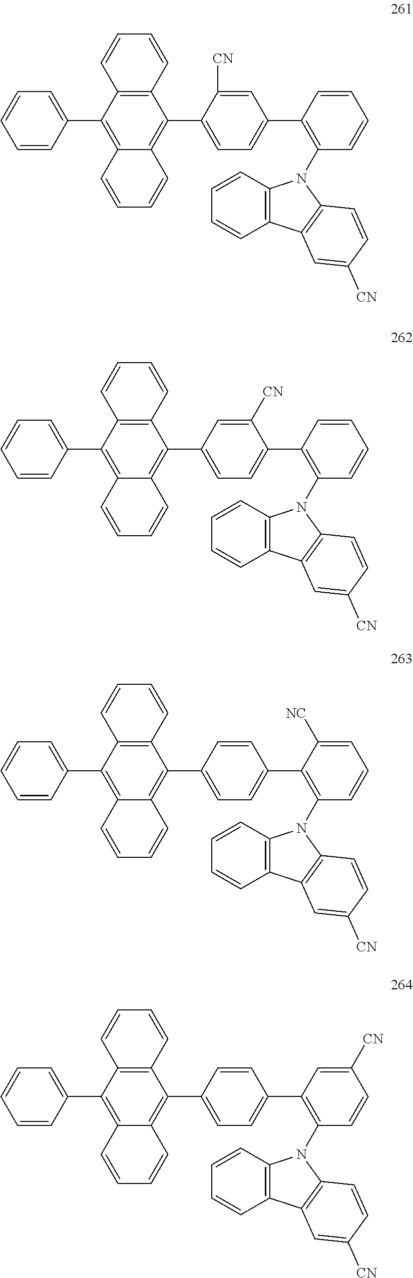 [SMILES: N#Cc1ccc2c(c1)c1ccccc1n2-c1ccccc1-c1ccc(-c2c3ccccc3c(-c3ccccc3)c3ccccc23)c(C#N)c1 ; N#Cc1ccc2c(c1)c1ccccc1n2-c1ccccc1-c1ccc(-c2c3ccccc3c(-c3ccccc3)c3ccccc23)cc1C#N ; N#Cc1ccc2c(c1)c1ccccc1n2-c1cccc(C#N)c1-c1ccc(-c2c3ccccc3c(-c3ccccc3)c3ccccc23)cc1 ; N#Cc1ccc(-n2c3ccccc3c3cc(C#N)ccc32)c(-c2ccc(-c3c4ccccc4c(-c4ccccc4)c4ccccc34)cc2)c1]

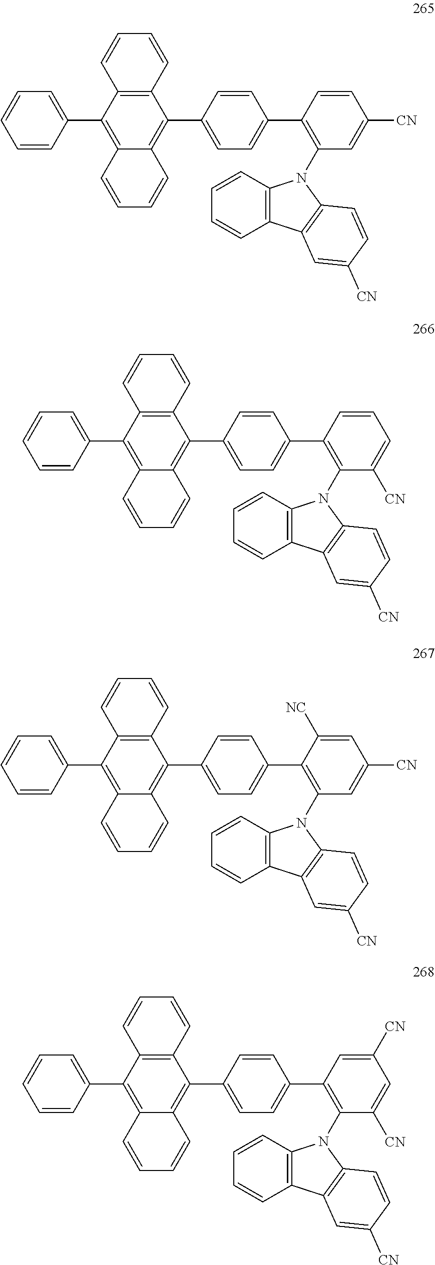 [SMILES: N#Cc1ccc(-c2ccc(-c3c4ccccc4c(-c4ccccc4)c4ccccc34)cc2)c(-n2c3ccccc3c3cc(C#N)ccc32)c1 ; N#Cc1ccc2c(c1)c1ccccc1n2-c1c(C#N)cccc1-c1ccc(-c2c3ccccc3c(-c3ccccc3)c3ccccc23)cc1 ; N#Cc1cc(C#N)c(-c2ccc(-c3c4ccccc4c(-c4ccccc4)c4ccccc34)cc2)c(-n2c3ccccc3c3cc(C#N)ccc32)c1 ; N#Cc1cc(C#N)c(-n2c3ccccc3c3cc(C#N)ccc32)c(-c2ccc(-c3c4ccccc4c(-c4ccccc4)c4ccccc34)cc2)c1]

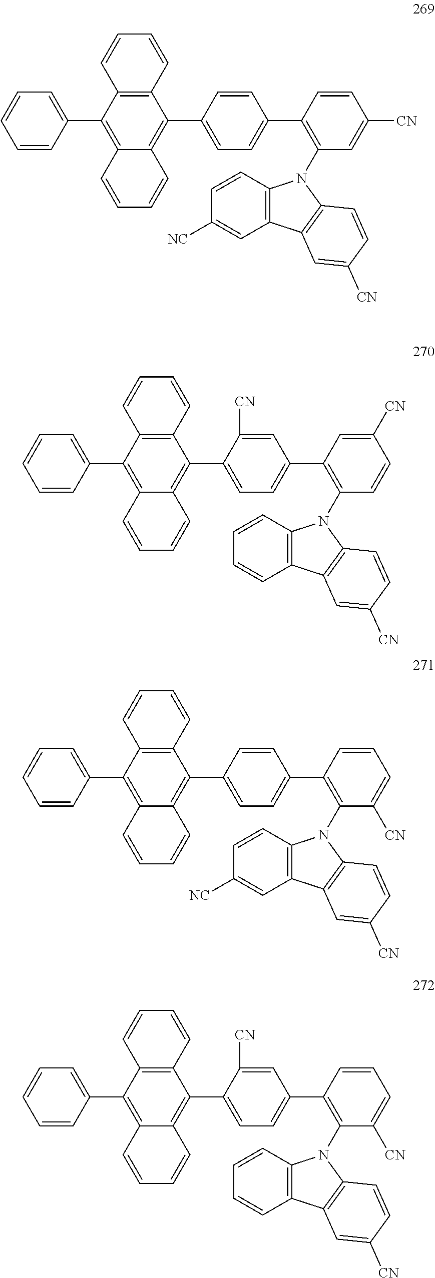 [SMILES: N#Cc1ccc(-c2ccc(-c3c4ccccc4c(-c4ccccc4)c4ccccc34)cc2)c(-n2c3ccc(C#N)cc3c3cc(C#N)ccc32)c1 ; N#Cc1ccc(-n2c3ccccc3c3cc(C#N)ccc32)c(-c2ccc(-c3c4ccccc4c(-c4ccccc4)c4ccccc34)c(C#N)c2)c1 ; N#Cc1ccc2c(c1)c1cc(C#N)ccc1n2-c1c(C#N)cccc1-c1ccc(-c2c3ccccc3c(-c3ccccc3)c3ccccc23)cc1 ; N#Cc1ccc2c(c1)c1ccccc1n2-c1c(C#N)cccc1-c1ccc(-c2c3ccccc3c(-c3ccccc3)c3ccccc23)c(C#N)c1]

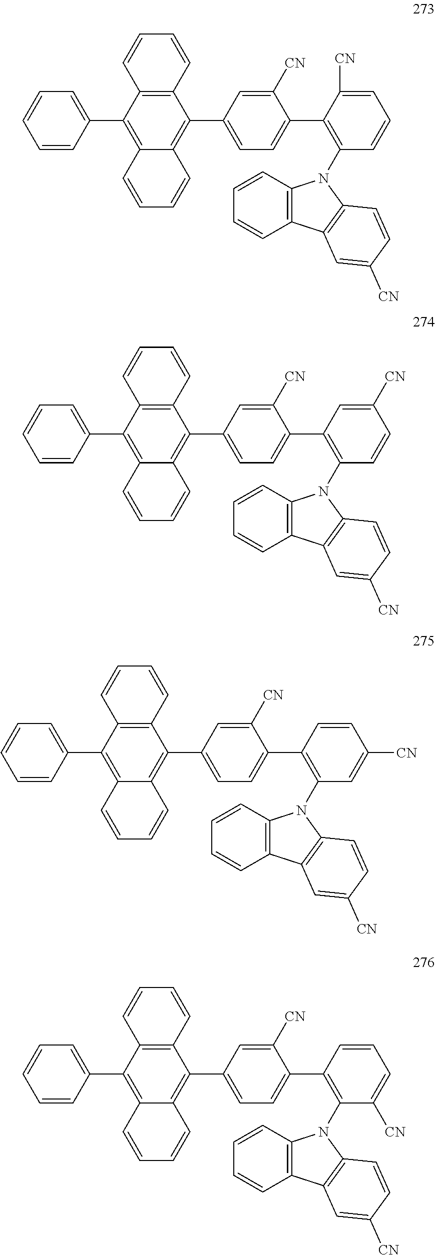 [SMILES: N#Cc1ccc2c(c1)c1ccccc1n2-c1cccc(C#N)c1-c1ccc(-c2c3ccccc3c(-c3ccccc3)c3ccccc23)cc1C#N ; N#Cc1ccc(-n2c3ccccc3c3cc(C#N)ccc32)c(-c2ccc(-c3c4ccccc4c(-c4ccccc4)c4ccccc34)cc2C#N)c1 ; N#Cc1ccc(-c2ccc(-c3c4ccccc4c(-c4ccccc4)c4ccccc34)cc2C#N)c(-n2c3ccccc3c3cc(C#N)ccc32)c1 ; N#Cc1ccc2c(c1)c1ccccc1n2-c1c(C#N)cccc1-c1ccc(-c2c3ccccc3c(-c3ccccc3)c3ccccc23)cc1C#N]

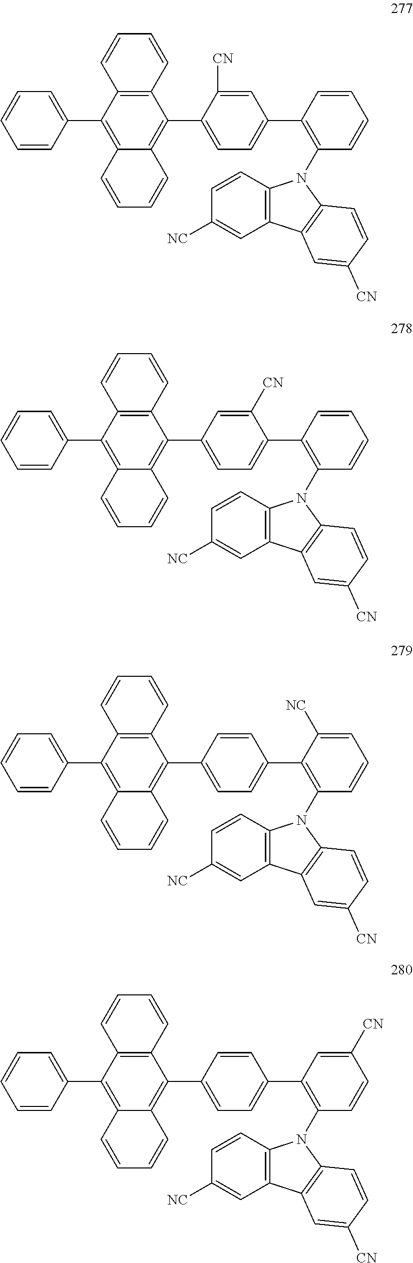 [SMILES: N#Cc1ccc2c(c1)c1cc(C#N)ccc1n2-c1ccccc1-c1ccc(-c2c3ccccc3c(-c3ccccc3)c3ccccc23)c(C#N)c1 ; N#Cc1ccc2c(c1)c1cc(C#N)ccc1n2-c1ccccc1-c1ccc(-c2c3ccccc3c(-c3ccccc3)c3ccccc23)cc1C#N ; N#Cc1ccc2c(c1)c1cc(C#N)ccc1n2-c1cccc(C#N)c1-c1ccc(-c2c3ccccc3c(-c3ccccc3)c3ccccc23)cc1 ; N#Cc1ccc(-n2c3ccc(C#N)cc3c3cc(C#N)ccc32)c(-c2ccc(-c3c4ccccc4c(-c4ccccc4)c4ccccc34)cc2)c1]

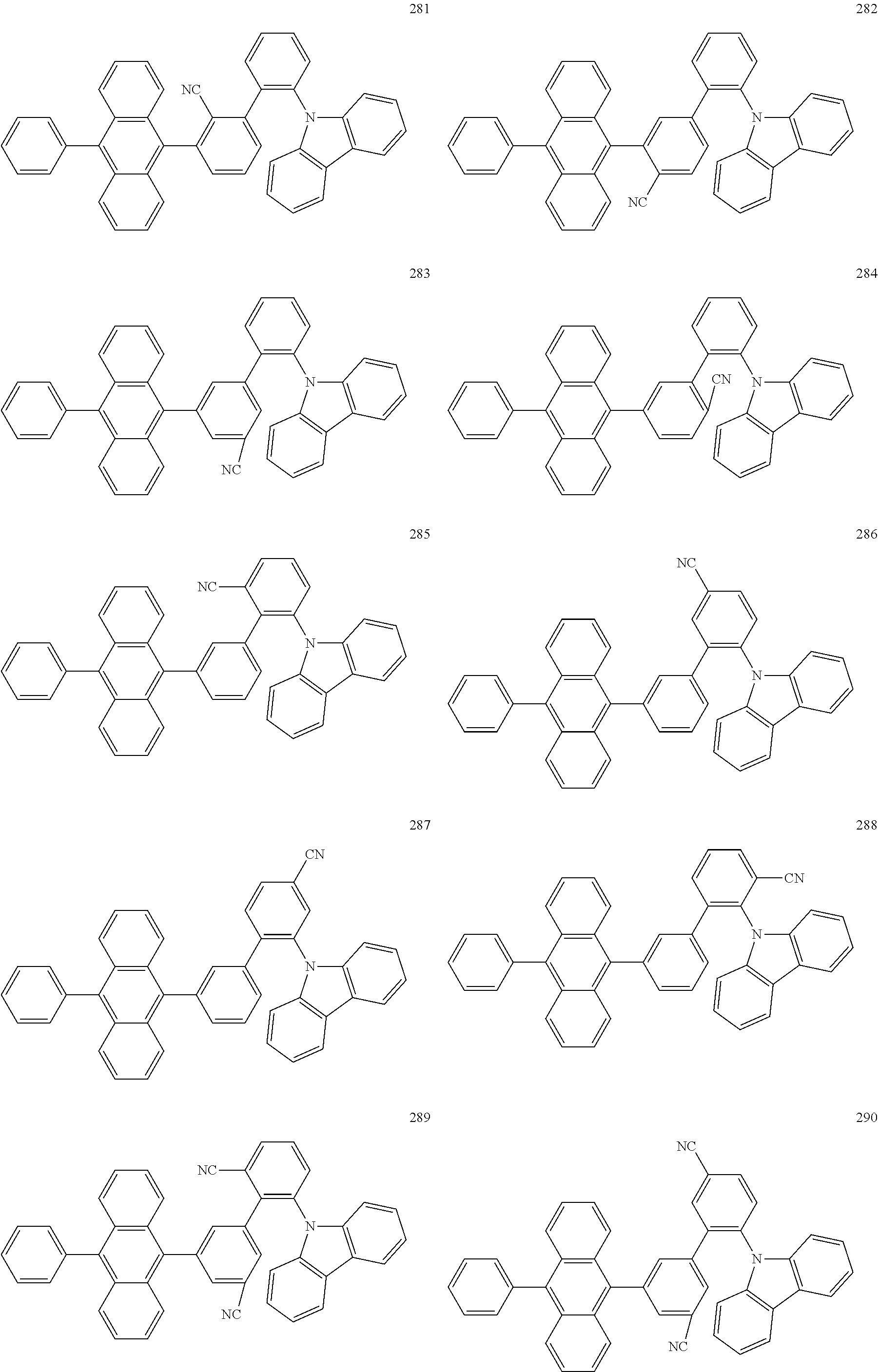

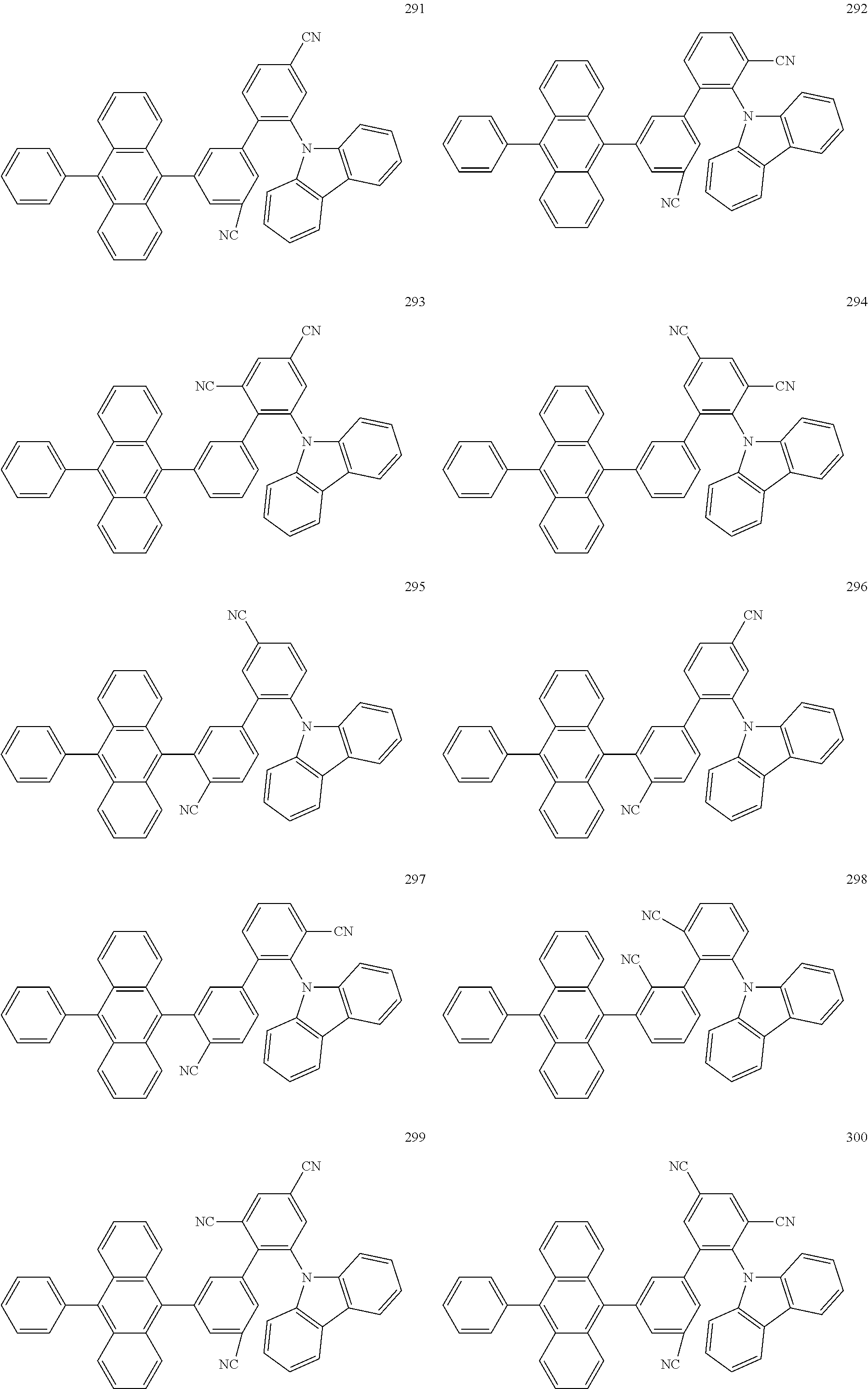

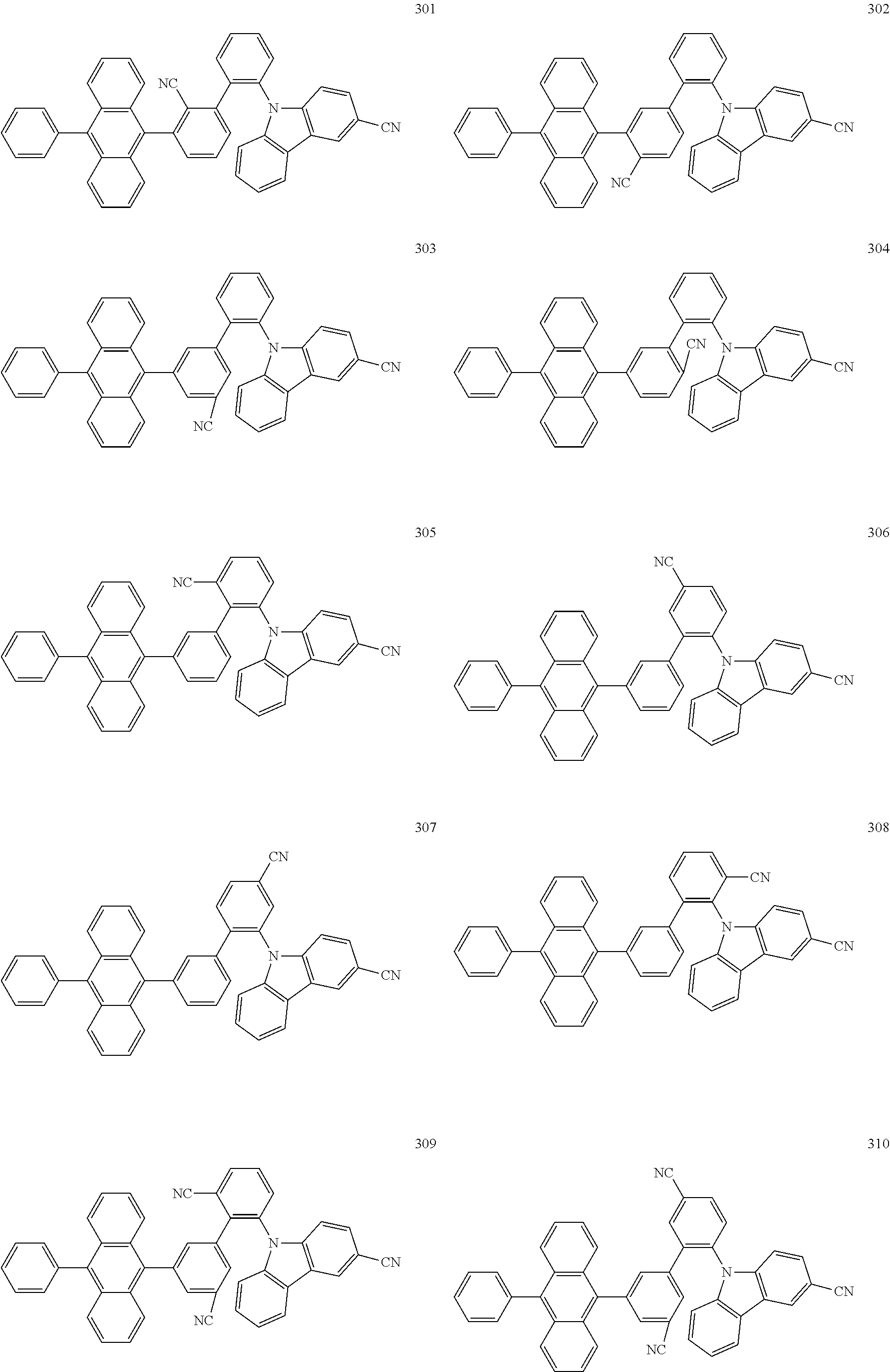

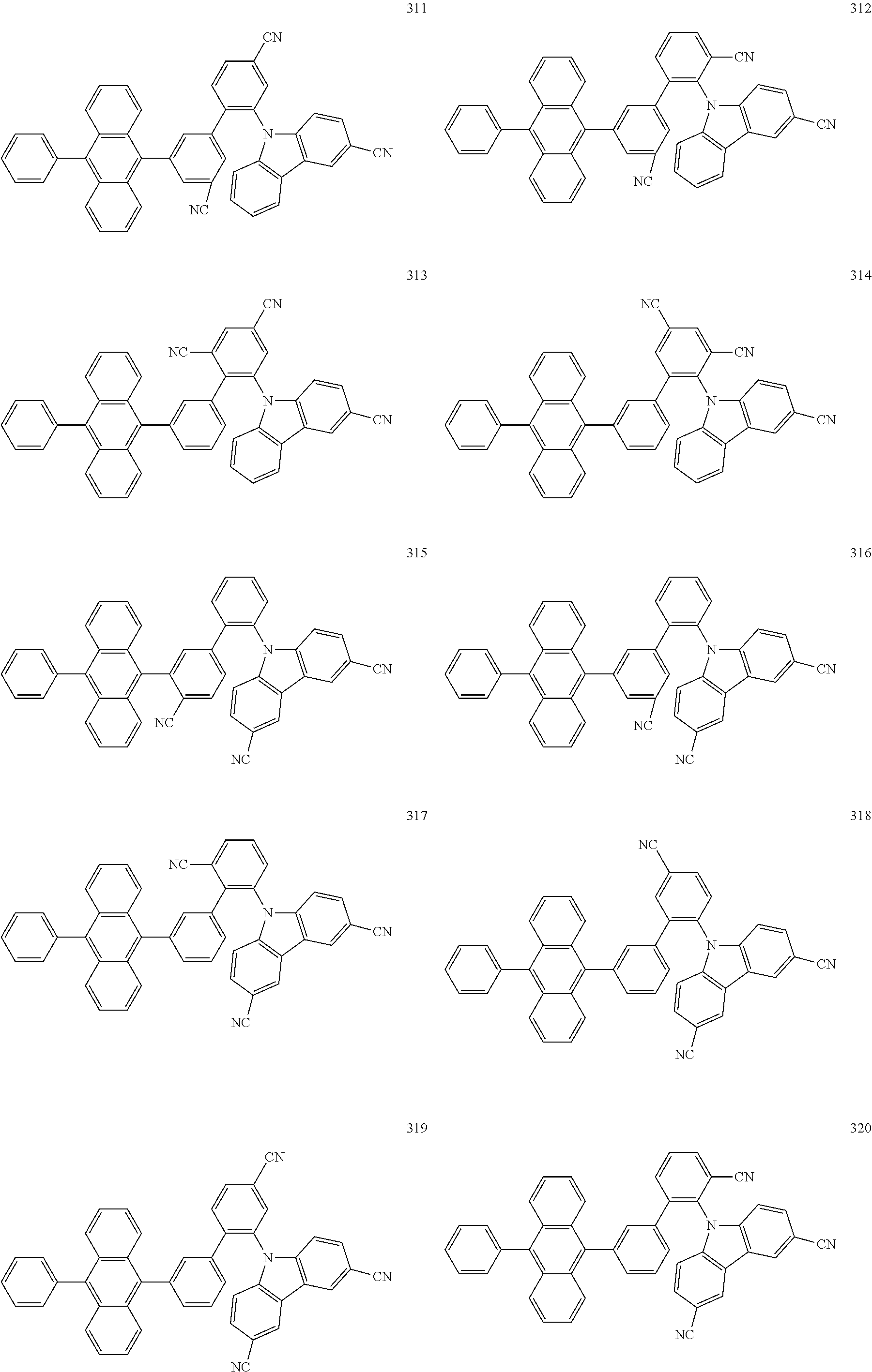

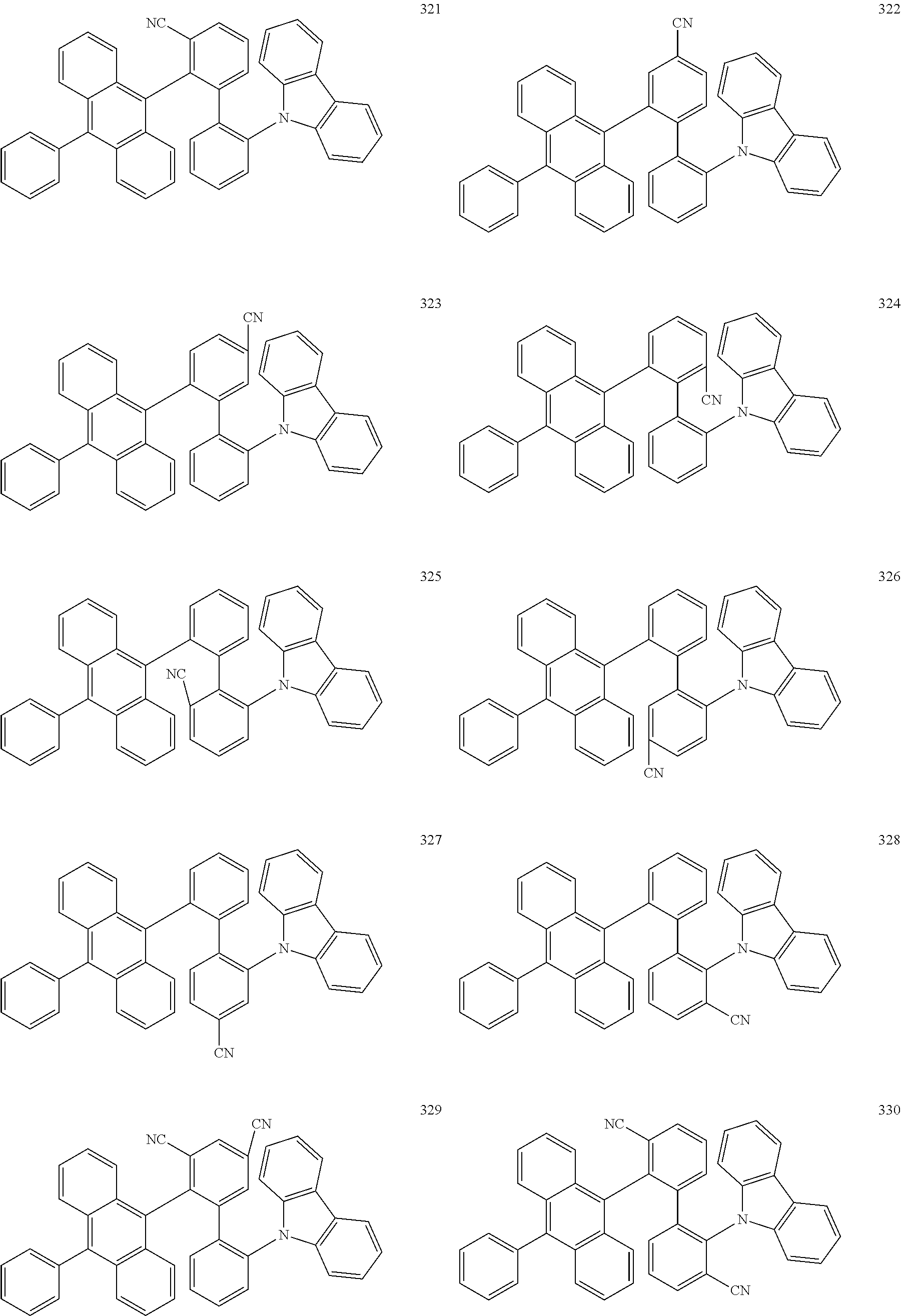

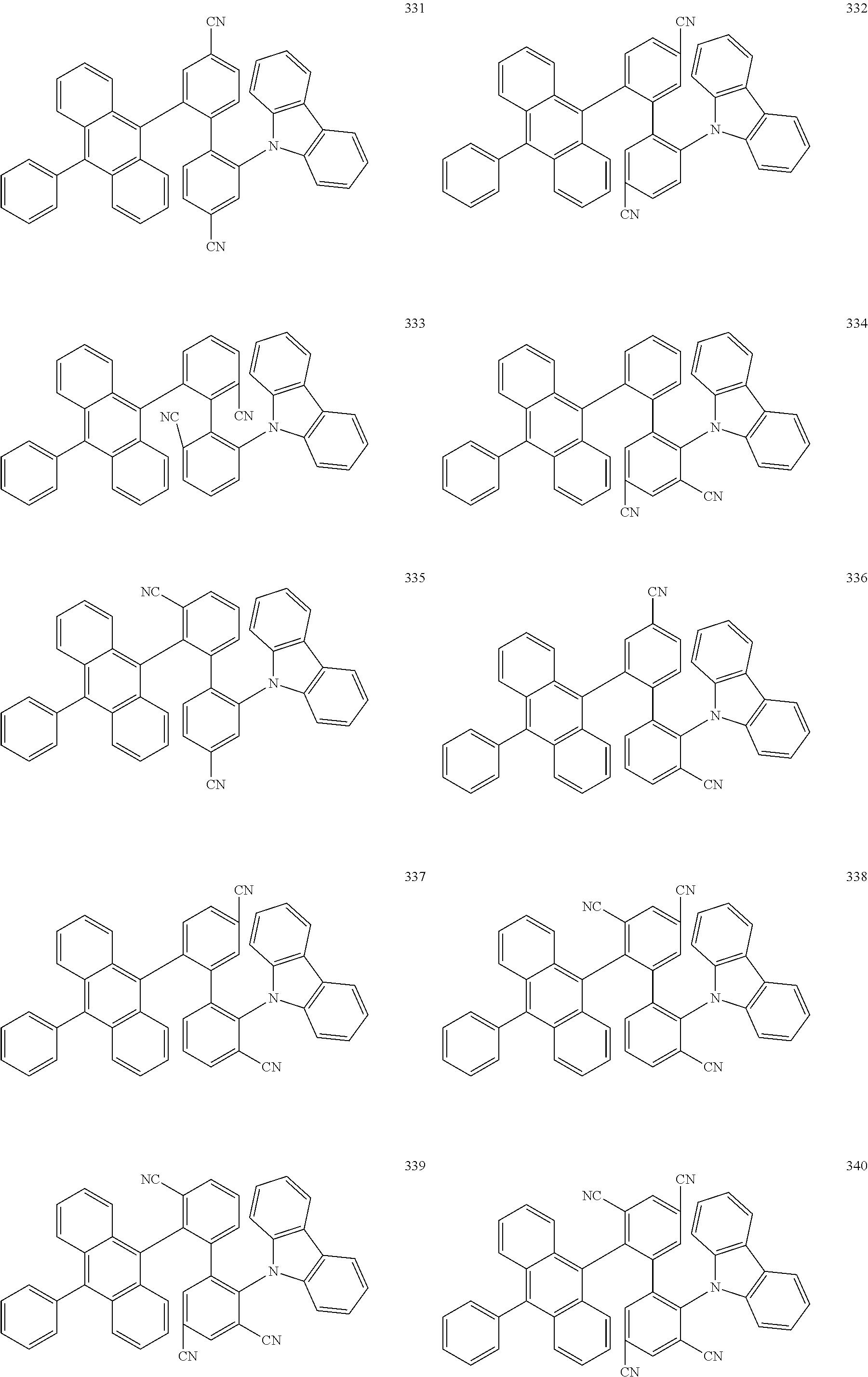

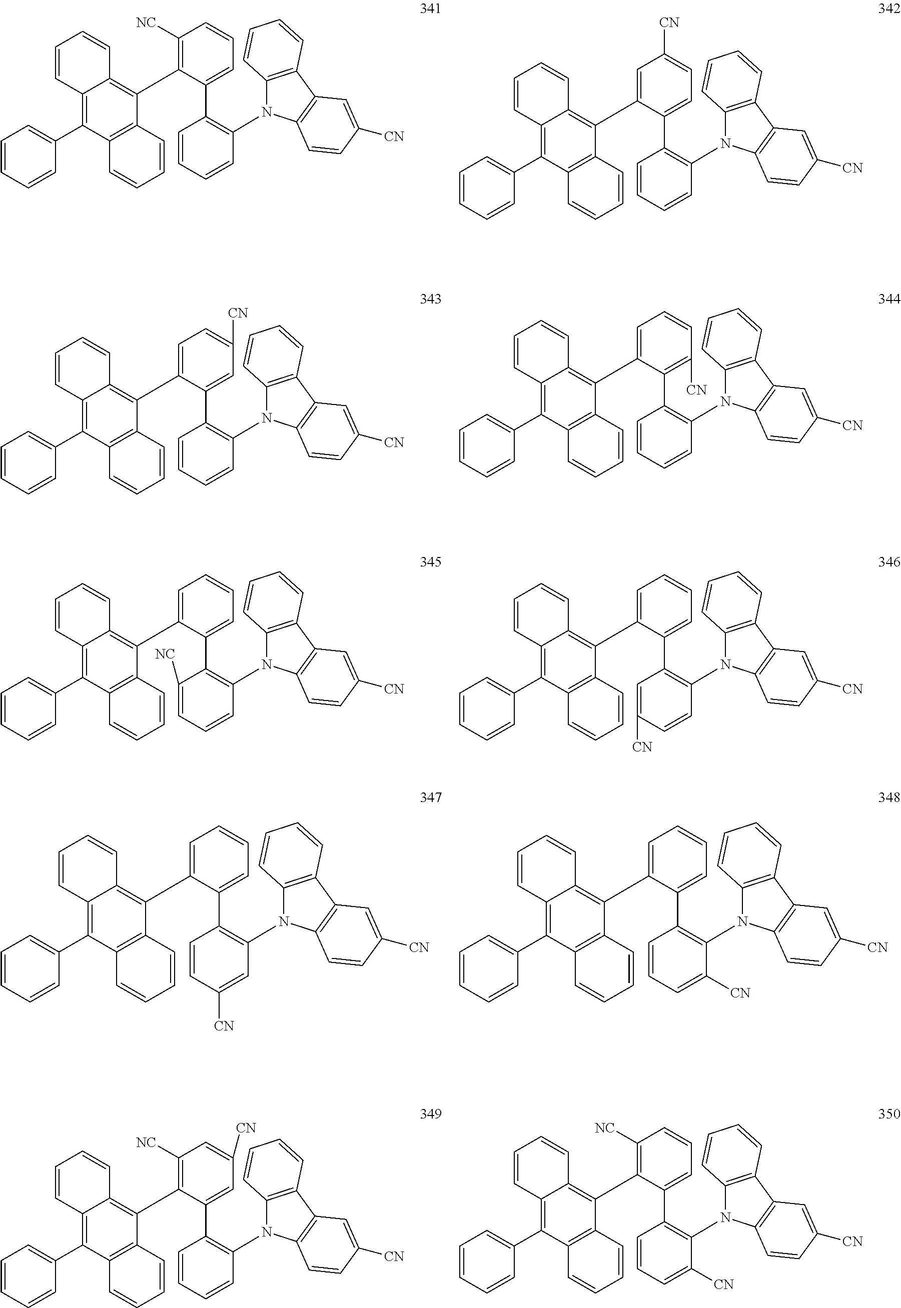

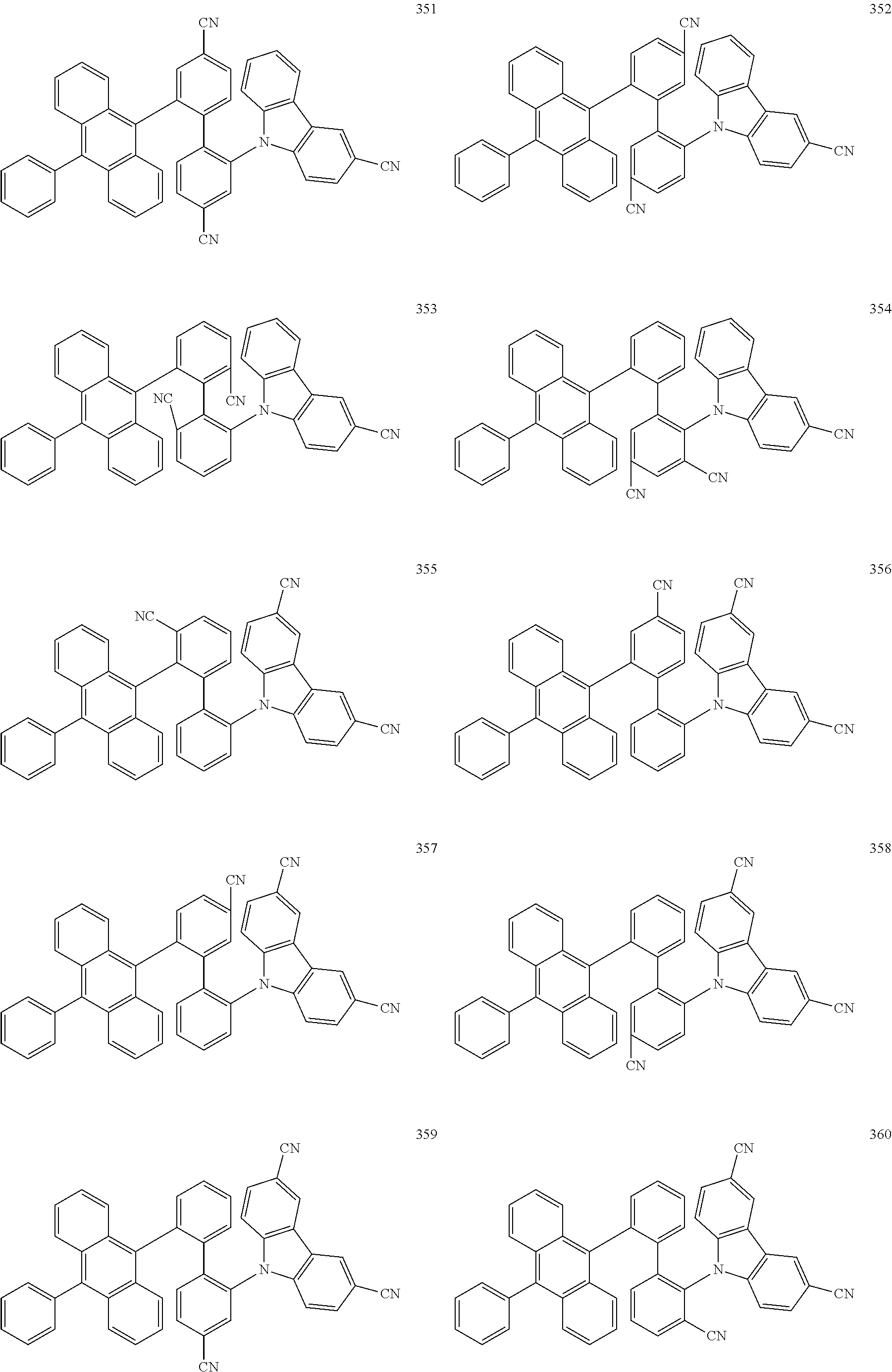

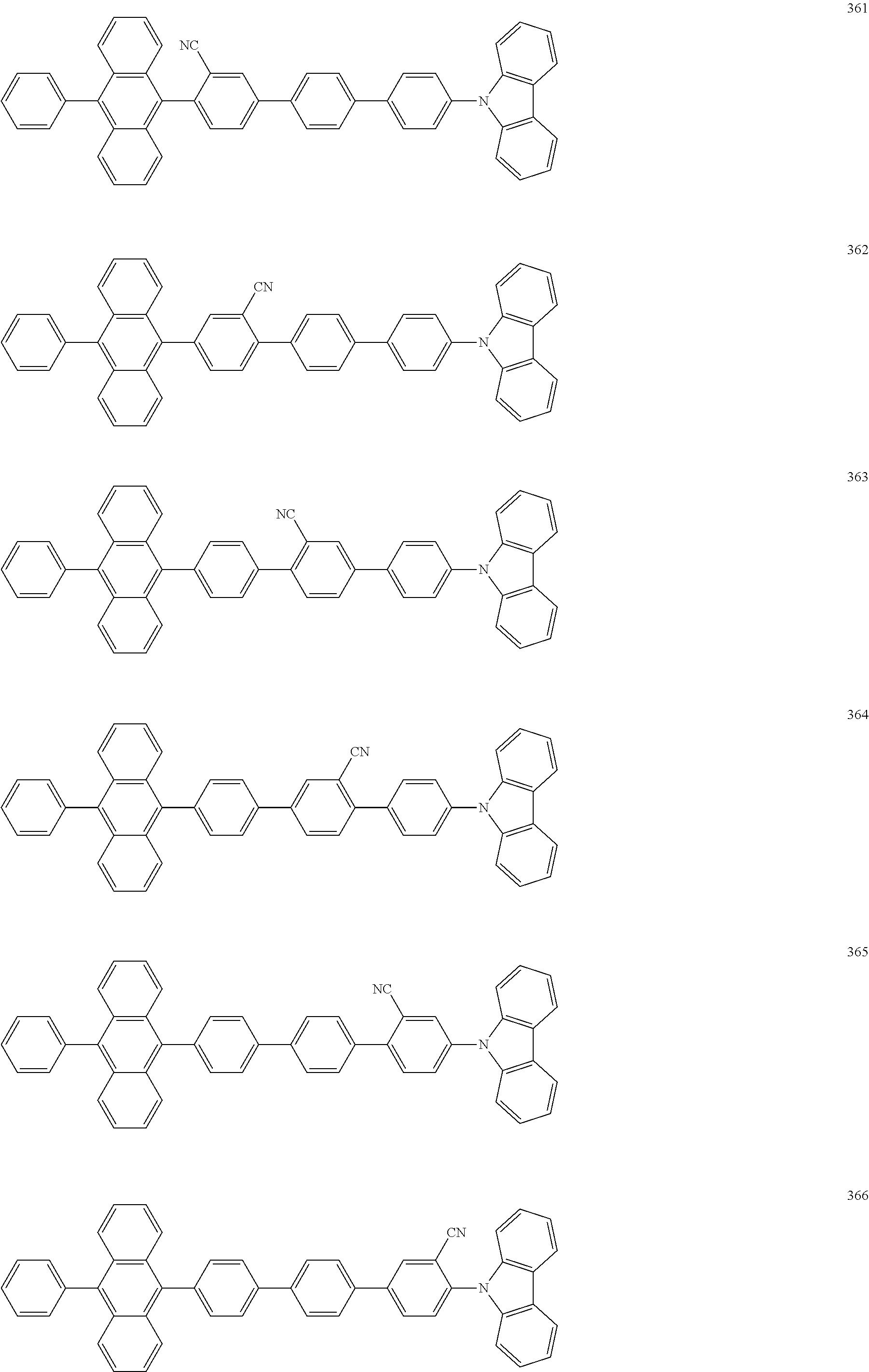

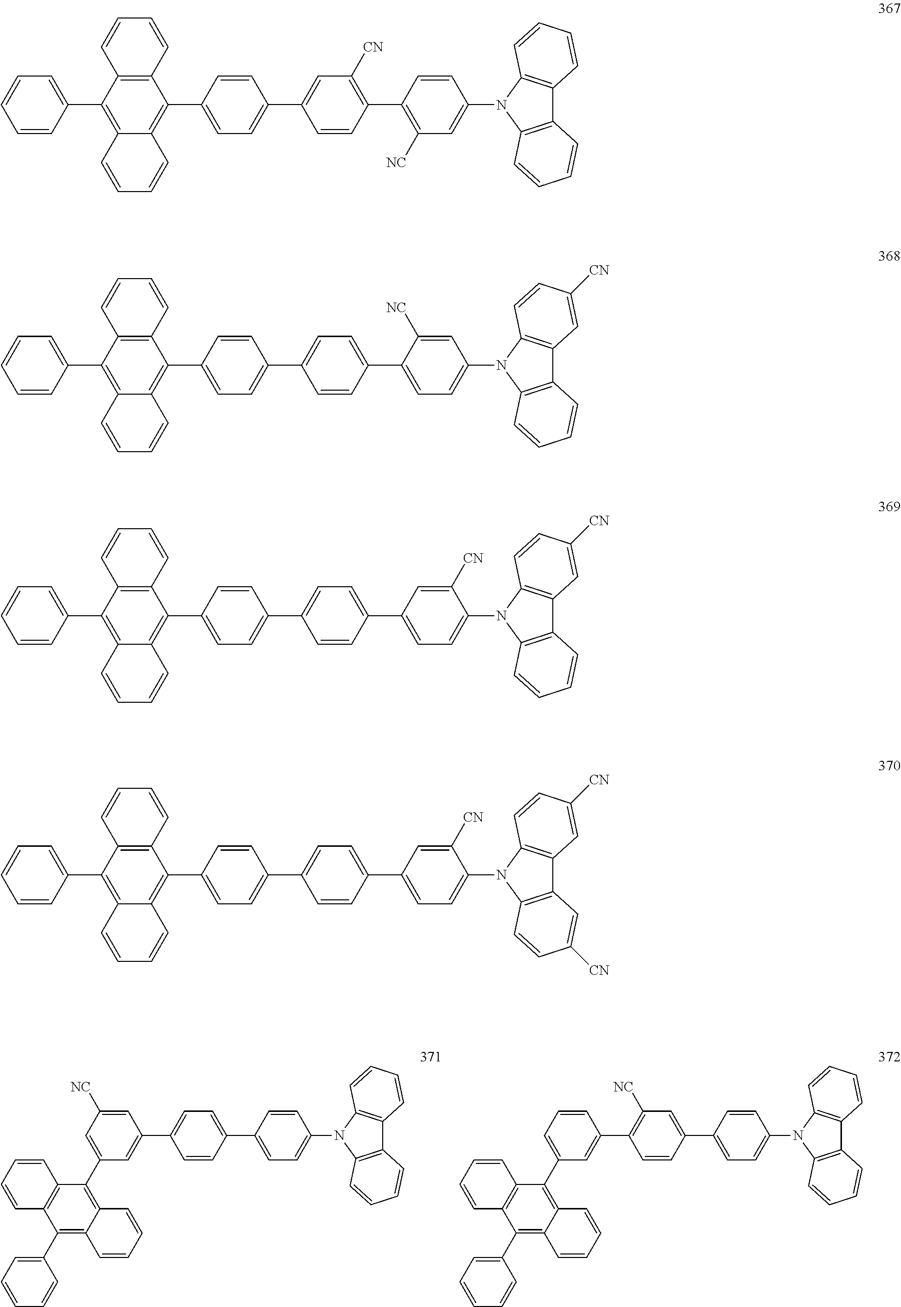

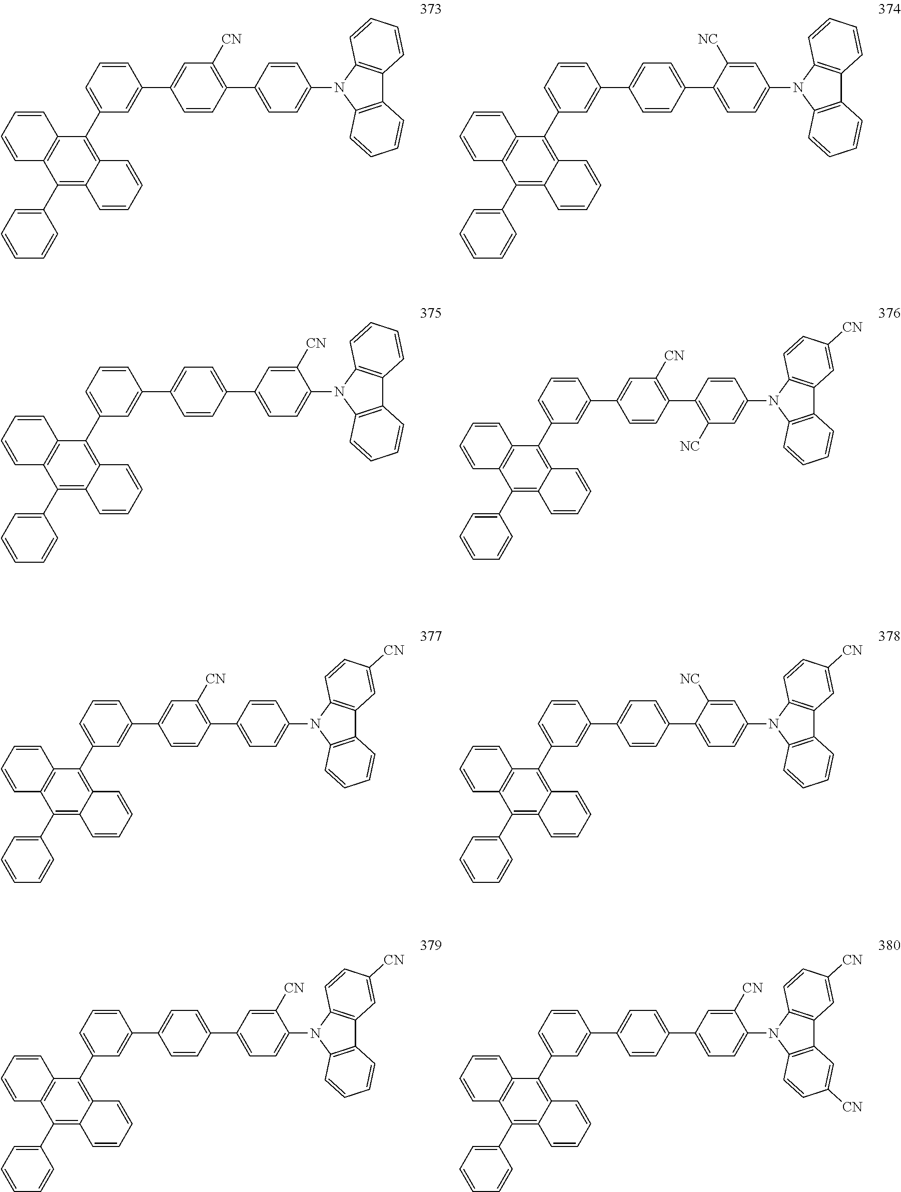

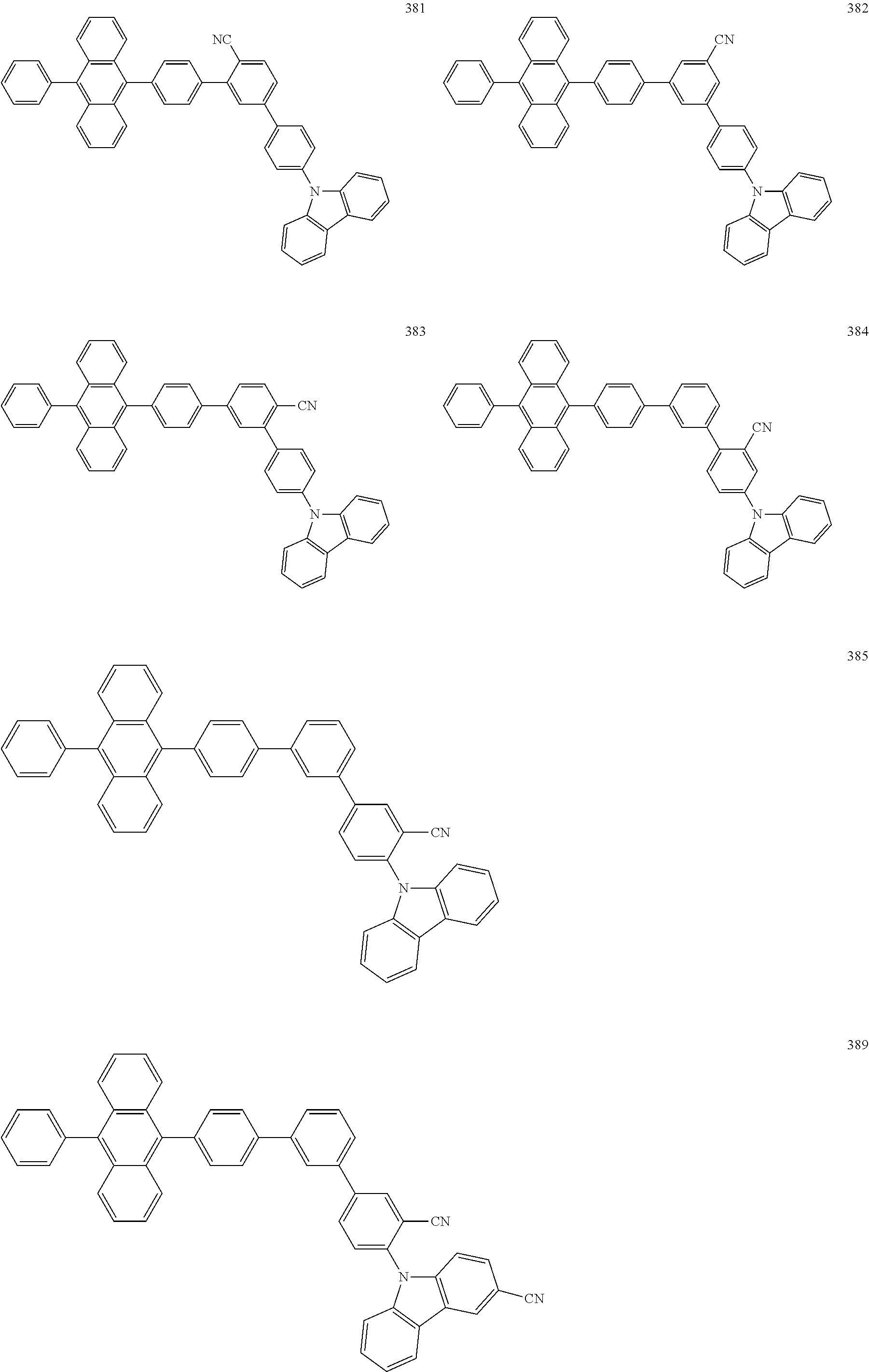

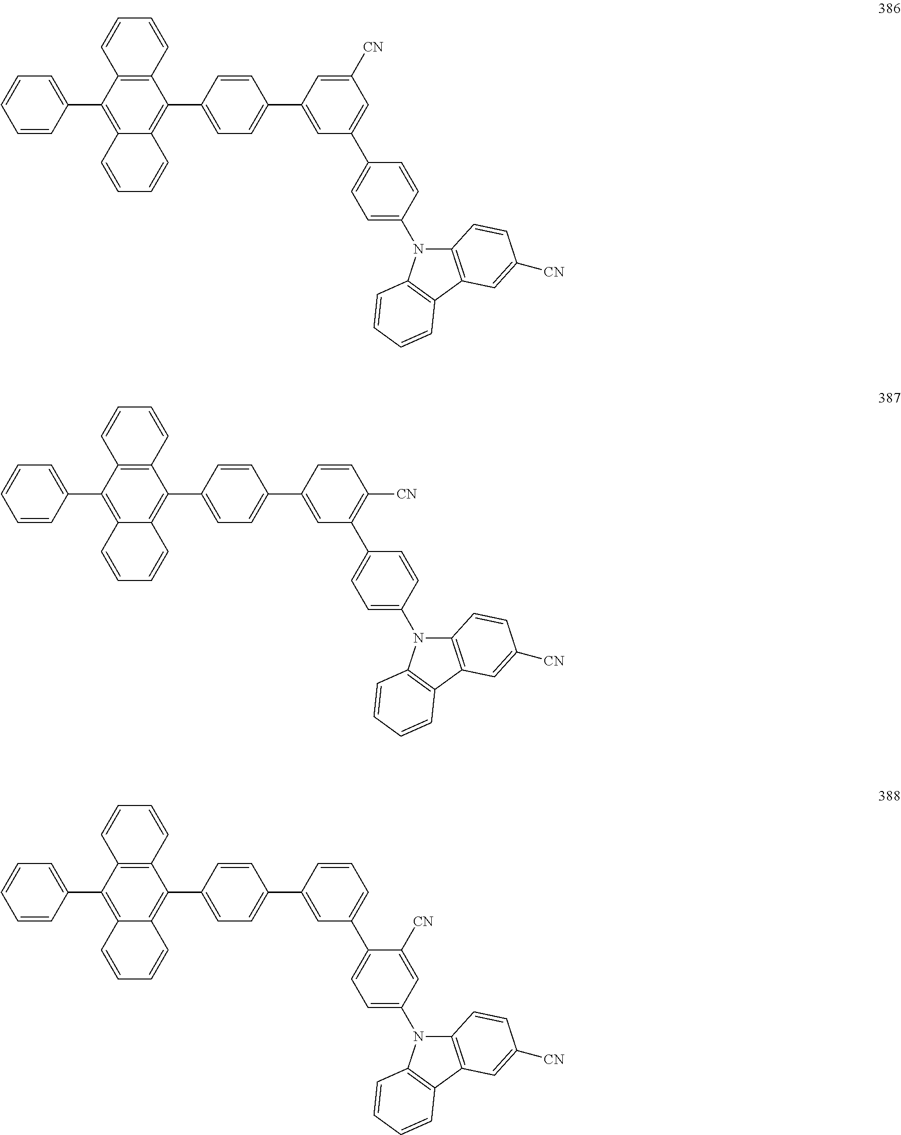 [SMILES: N#Cc1cc(-c2ccc(-c3c4ccccc4c(-c4ccccc4)c4ccccc34)cc2)cc(-c2ccc(-n3c4ccccc4c4cc(C#N)ccc43)cc2)c1 ; N#Cc1ccc2c(c1)c1ccccc1n2-c1ccc(-c2cc(-c3ccc(-c4c5ccccc5c(-c5ccccc5)c5ccccc45)cc3)ccc2C#N)cc1 ; N#Cc1ccc2c(c1)c1ccccc1n2-c1ccc(-c2cccc(-c3ccc(-c4c5ccccc5c(-c5ccccc5)c5ccccc45)cc3)c2)c(C#N)c1]

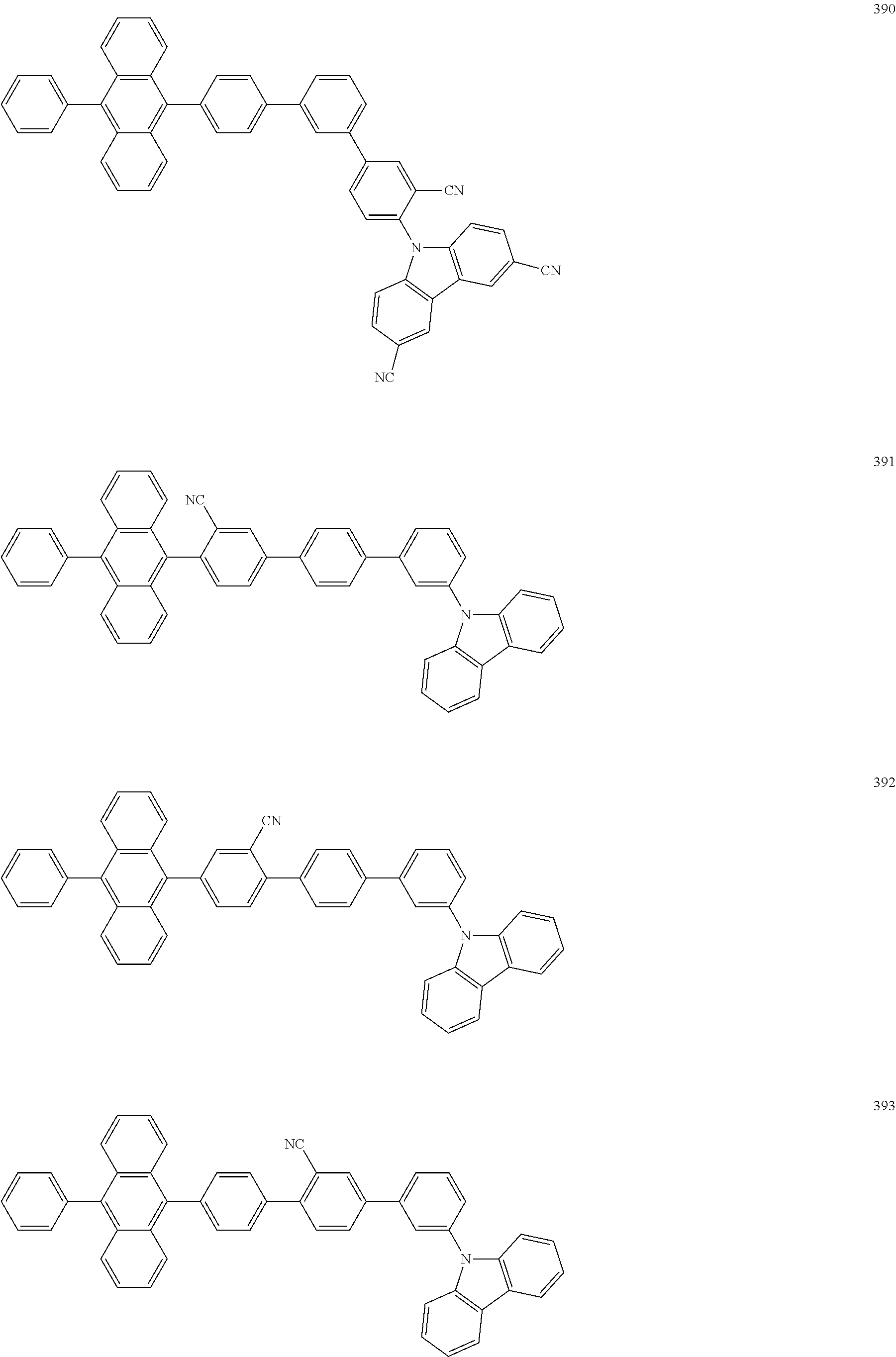 [SMILES: N#Cc1ccc2c(c1)c1cc(C#N)ccc1n2-c1ccc(-c2cccc(-c3ccc(-c4c5ccccc5c(-c5ccccc5)c5ccccc45)cc3)c2)cc1C#N ; N#Cc1cc(-c2ccc(-c3cccc(-n4c5ccccc5c5ccccc54)c3)cc2)ccc1-c1c2ccccc2c(-c2ccccc2)c2ccccc12 ; N#Cc1cc(-c2c3ccccc3c(-c3ccccc3)c3ccccc23)ccc1-c1ccc(-c2cccc(-n3c4ccccc4c4ccccc43)c2)cc1 ; N#Cc1cc(-c2cccc(-n3c4ccccc4c4ccccc43)c2)ccc1-c1ccc(-c2c3ccccc3c(-c3ccccc3)c3ccccc23)cc1]

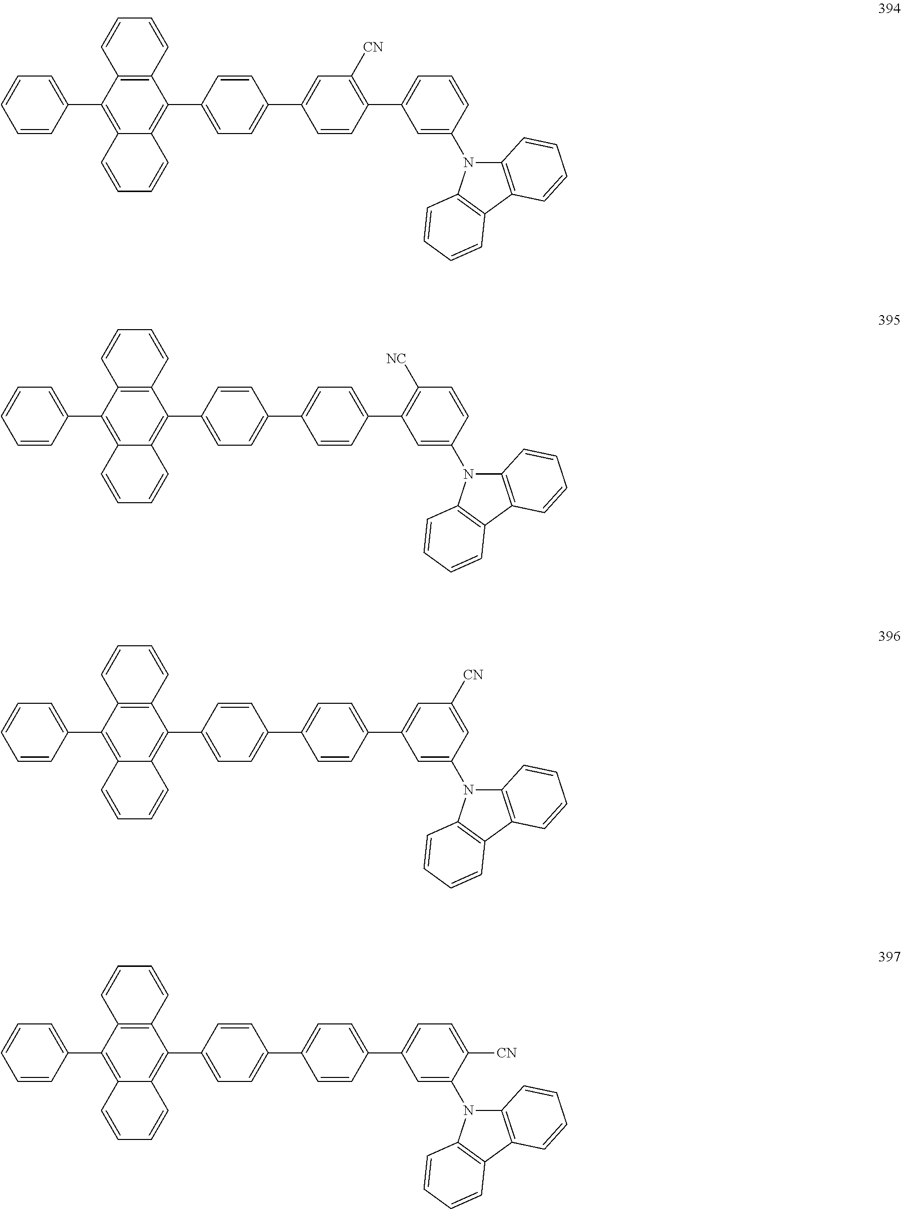 [SMILES: N#Cc1cc(-c2ccc(-c3c4ccccc4c(-c4ccccc4)c4ccccc34)cc2)ccc1-c1cccc(-n2c3ccccc3c3ccccc32)c1 ; N#Cc1ccc(-n2c3ccccc3c3ccccc32)cc1-c1ccc(-c2ccc(-c3c4ccccc4c(-c4ccccc4)c4ccccc34)cc2)cc1 ; N#Cc1cc(-c2ccc(-c3ccc(-c4c5ccccc5c(-c5ccccc5)c5ccccc45)cc3)cc2)cc(-n2c3ccccc3c3ccccc32)c1 ; N#Cc1ccc(-c2ccc(-c3ccc(-c4c5ccccc5c(-c5ccccc5)c5ccccc45)cc3)cc2)cc1-n1c2ccccc2c2ccccc21]

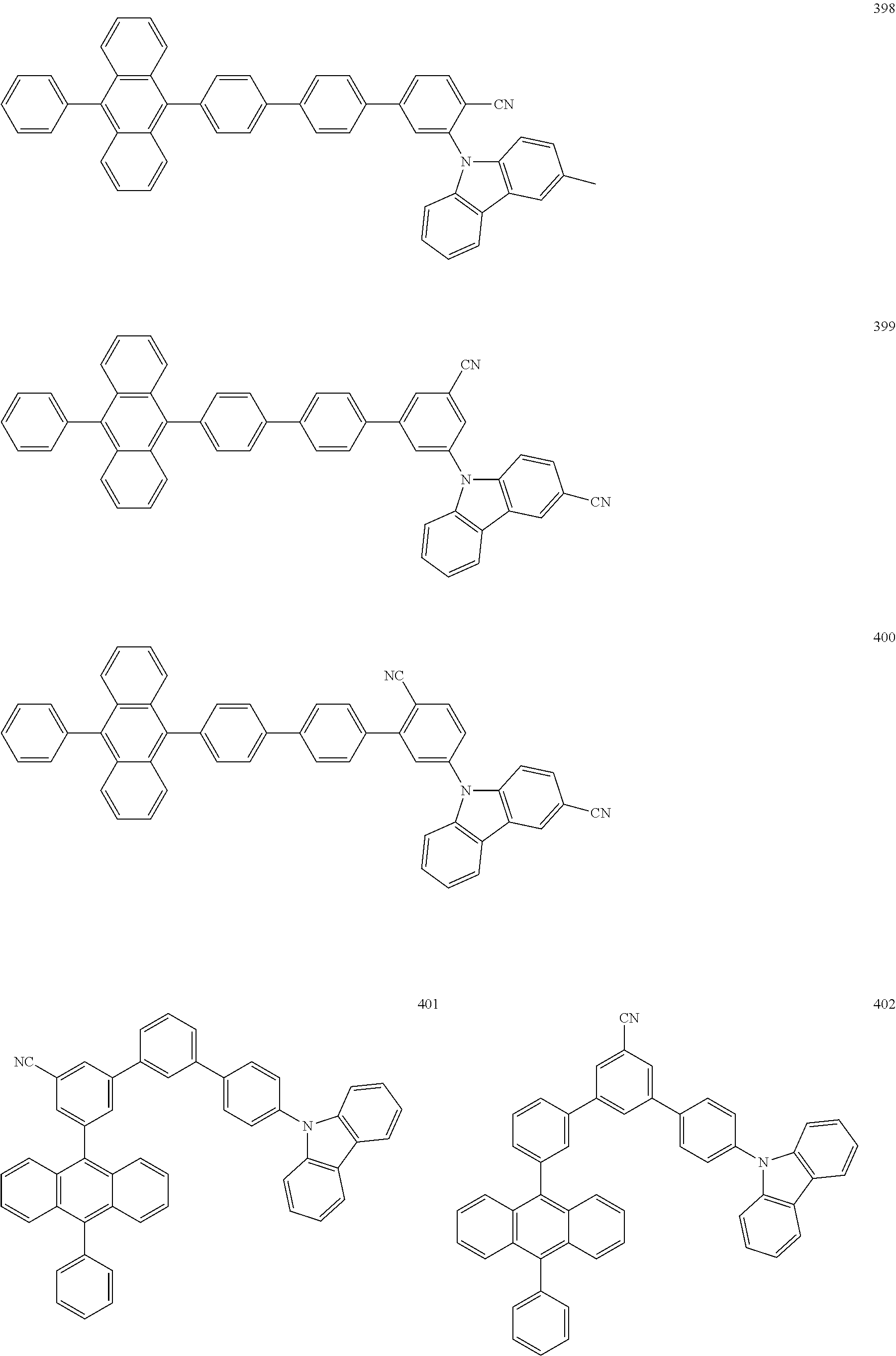 [SMILES: Cc1ccc2c(c1)c1ccccc1n2-c1cc(-c2ccc(-c3ccc(-c4c5ccccc5c(-c5ccccc5)c5ccccc45)cc3)cc2)ccc1C#N ; N#Cc1cc(-c2ccc(-c3ccc(-c4c5ccccc5c(-c5ccccc5)c5ccccc45)cc3)cc2)cc(-n2c3ccccc3c3cc(C#N)ccc32)c1 ; N#Cc1ccc2c(c1)c1ccccc1n2-c1ccc(C#N)c(-c2ccc(-c3ccc(-c4c5ccccc5c(-c5ccccc5)c5ccccc45)cc3)cc2)c1 ; N#Cc1cc(-c2cccc(-c3ccc(-n4c5ccccc5c5ccccc54)cc3)c2)cc(-c2c3ccccc3c(-c3ccccc3)c3ccccc23)c1 ; N#Cc1cc(-c2ccc(-n3c4ccccc4c4ccccc43)cc2)cc(-c2cccc(-c3c4ccccc4c(-c4ccccc4)c4ccccc34)c2)c1]

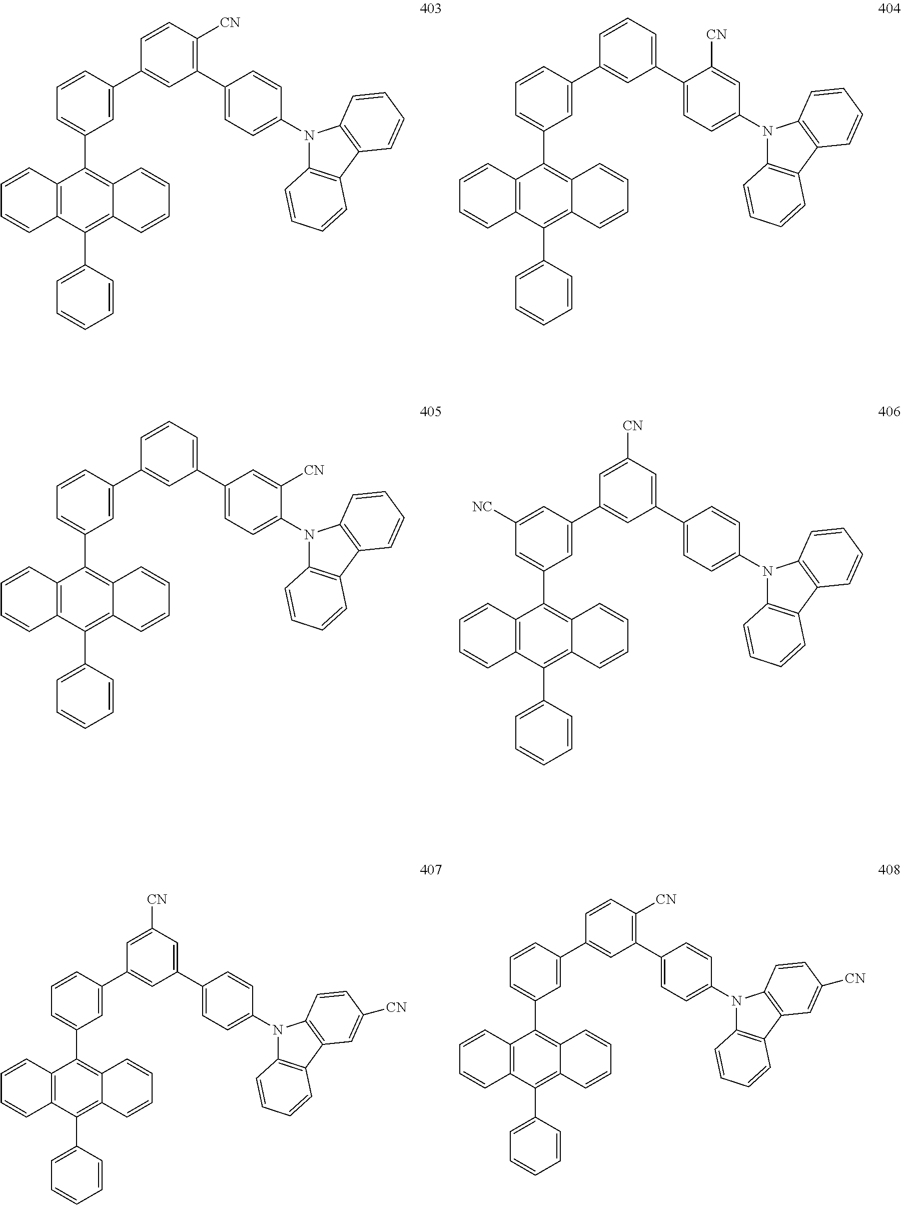

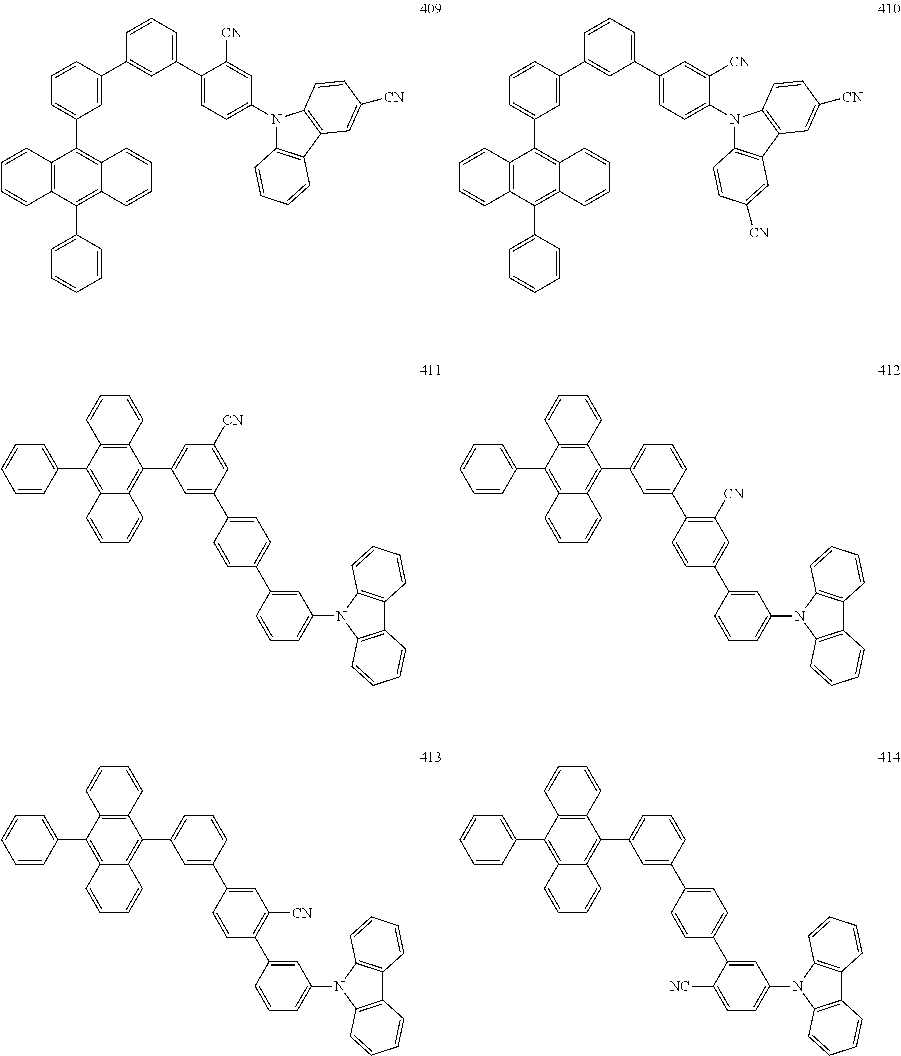

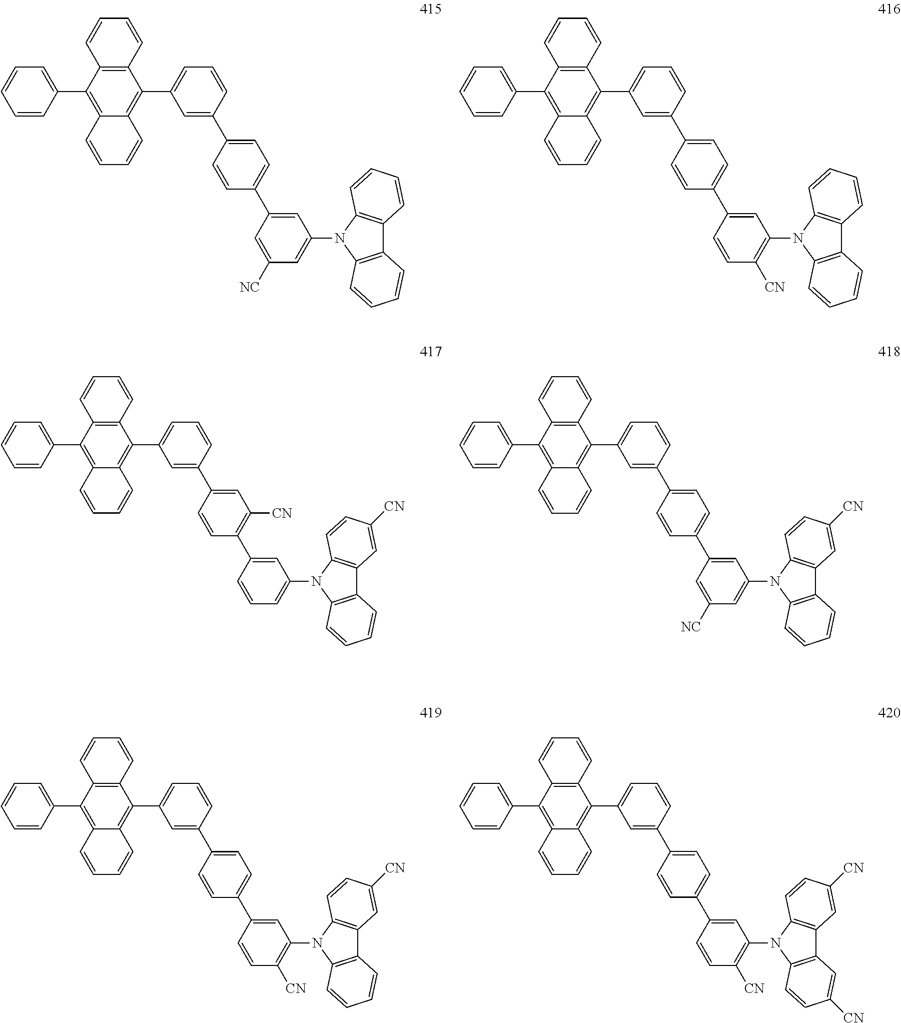

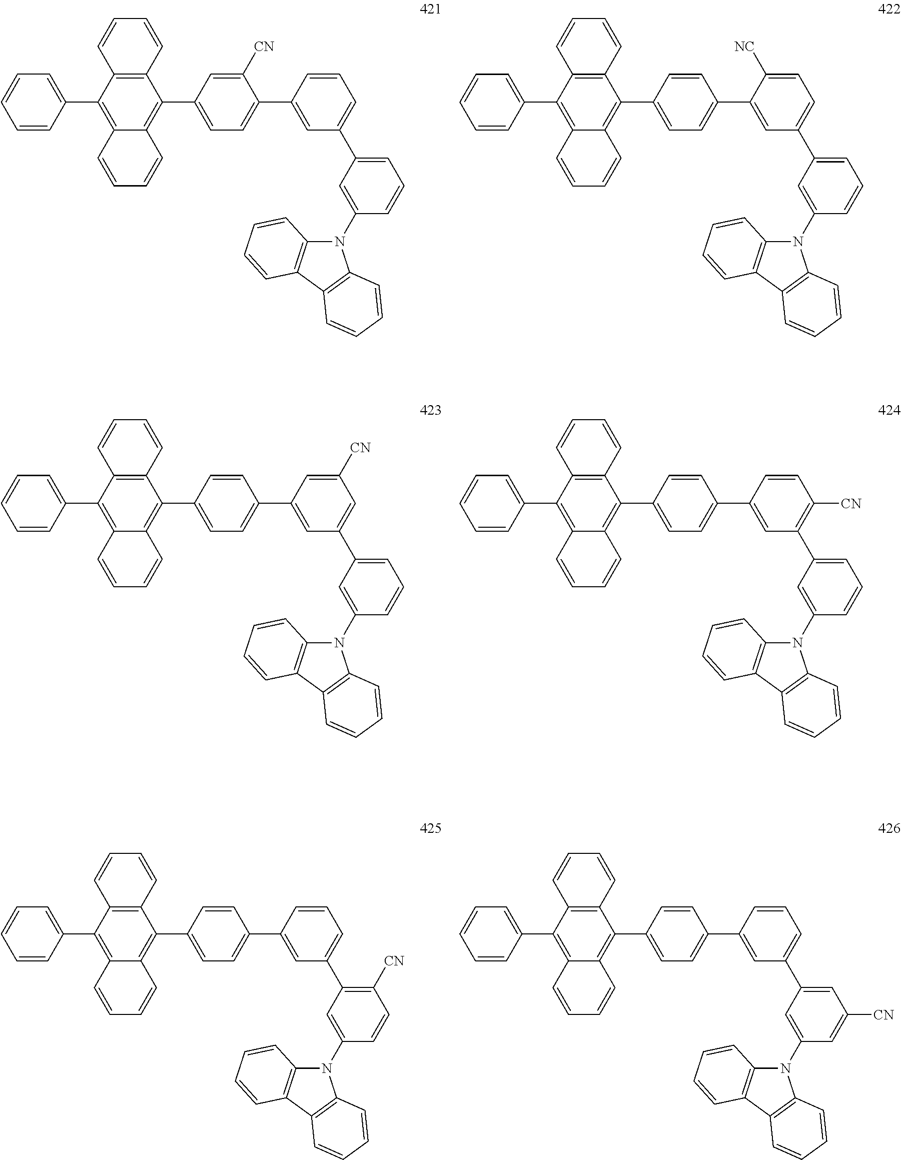

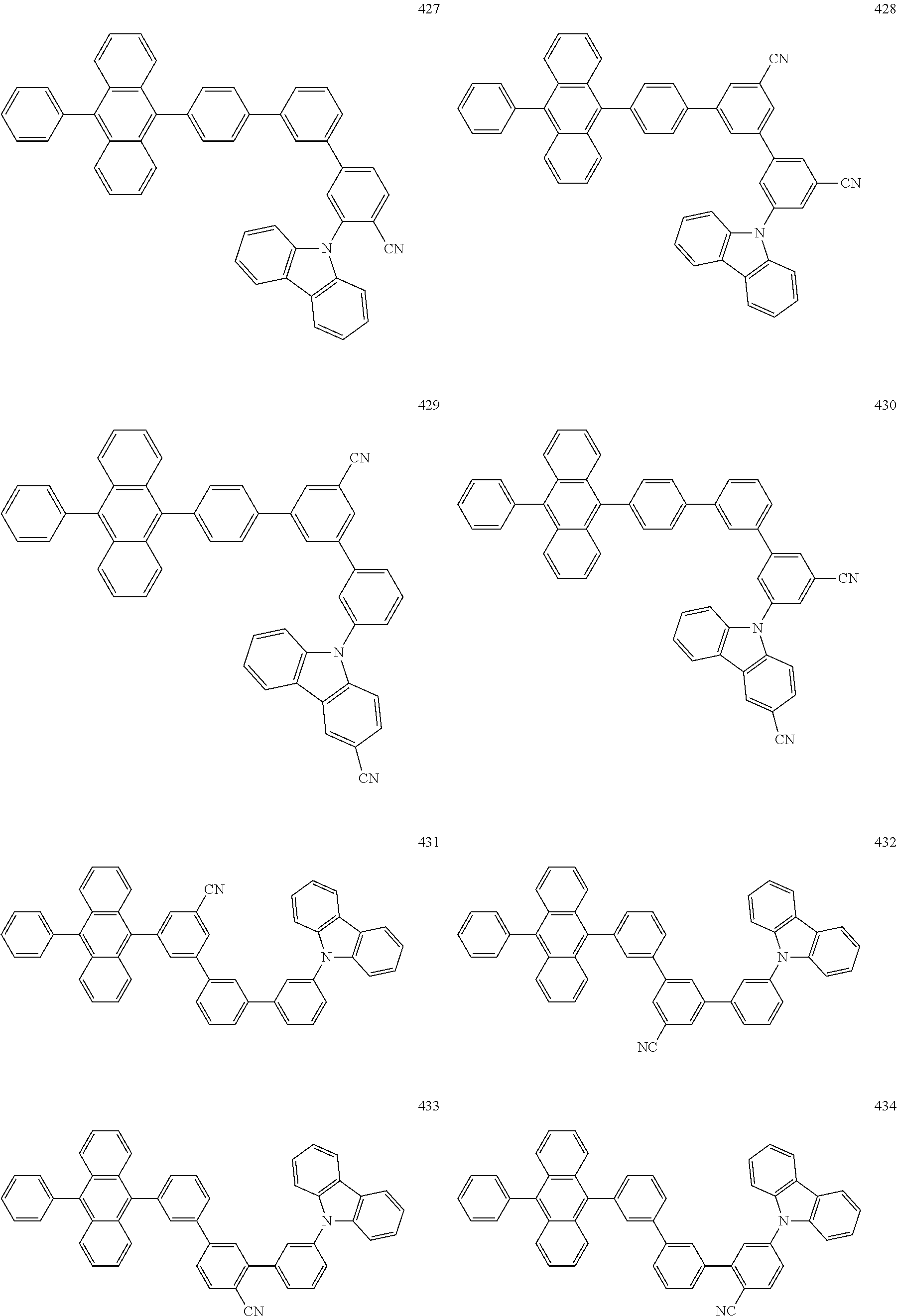

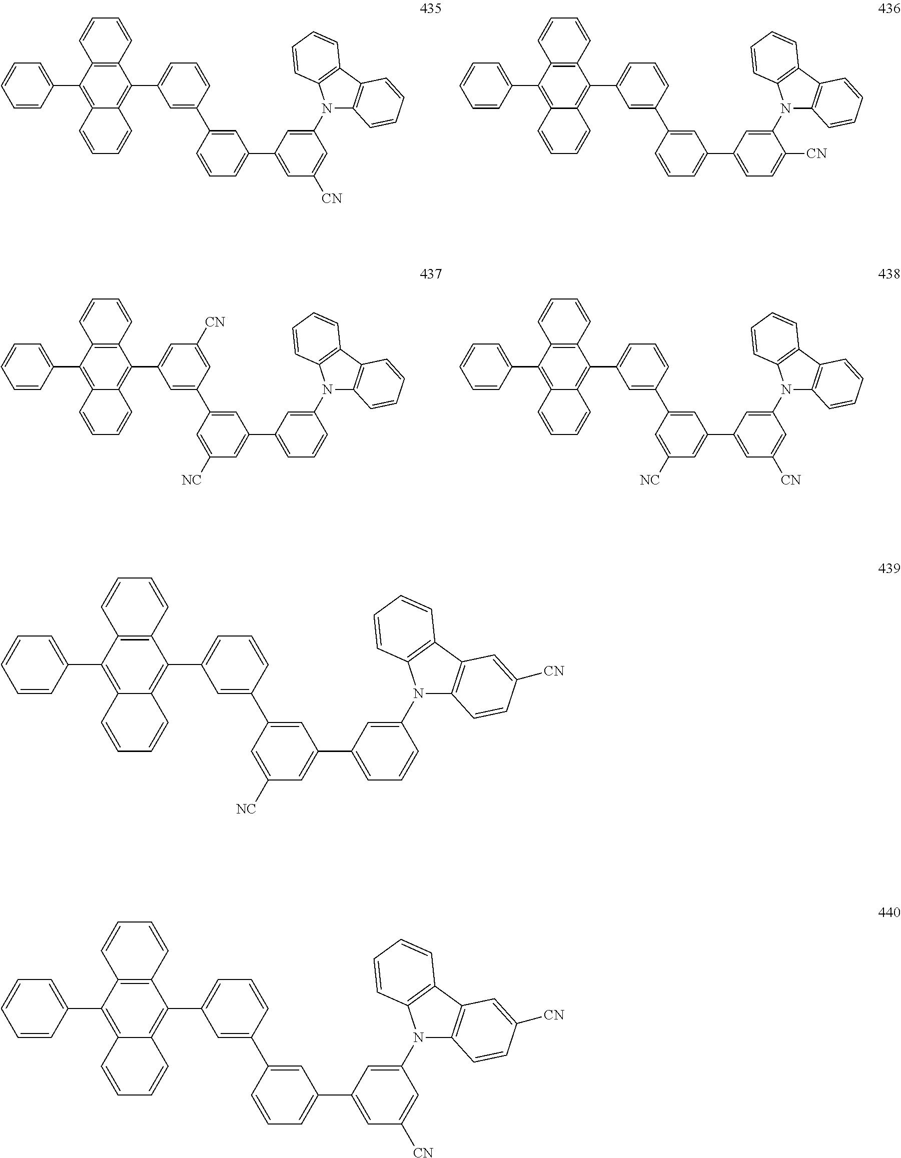

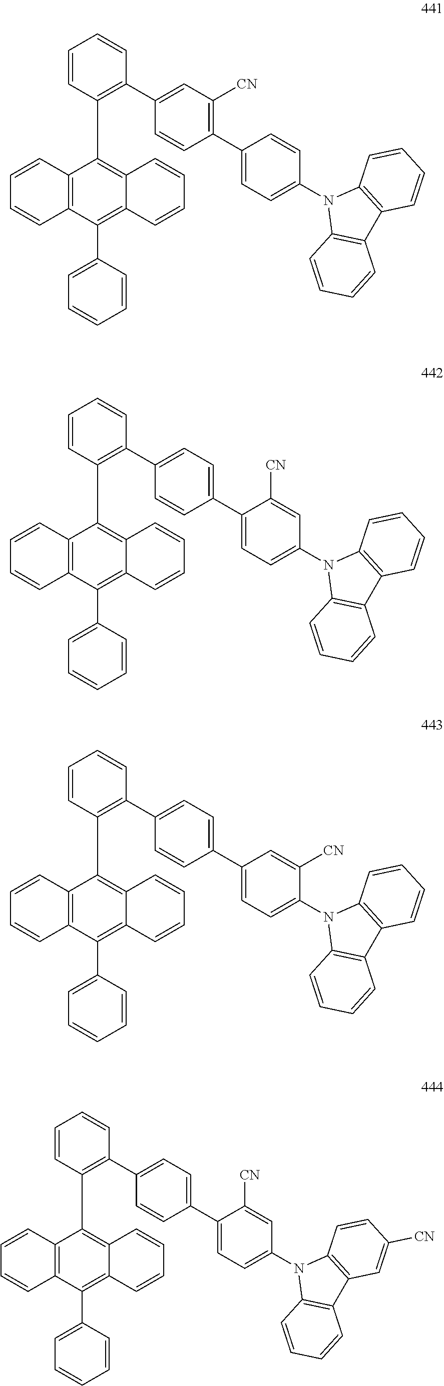 [SMILES: N#Cc1cc(-c2ccccc2-c2c3ccccc3c(-c3ccccc3)c3ccccc23)ccc1-c1ccc(-n2c3ccccc3c3ccccc32)cc1 ; N#Cc1cc(-n2c3ccccc3c3ccccc32)ccc1-c1ccc(-c2ccccc2-c2c3ccccc3c(-c3ccccc3)c3ccccc23)cc1 ; N#Cc1cc(-c2ccc(-c3ccccc3-c3c4ccccc4c(-c4ccccc4)c4ccccc34)cc2)ccc1-n1c2ccccc2c2ccccc21 ; N#Cc1ccc2c(c1)c1ccccc1n2-c1ccc(-c2ccc(-c3ccccc3-c3c4ccccc4c(-c4ccccc4)c4ccccc34)cc2)c(C#N)c1]

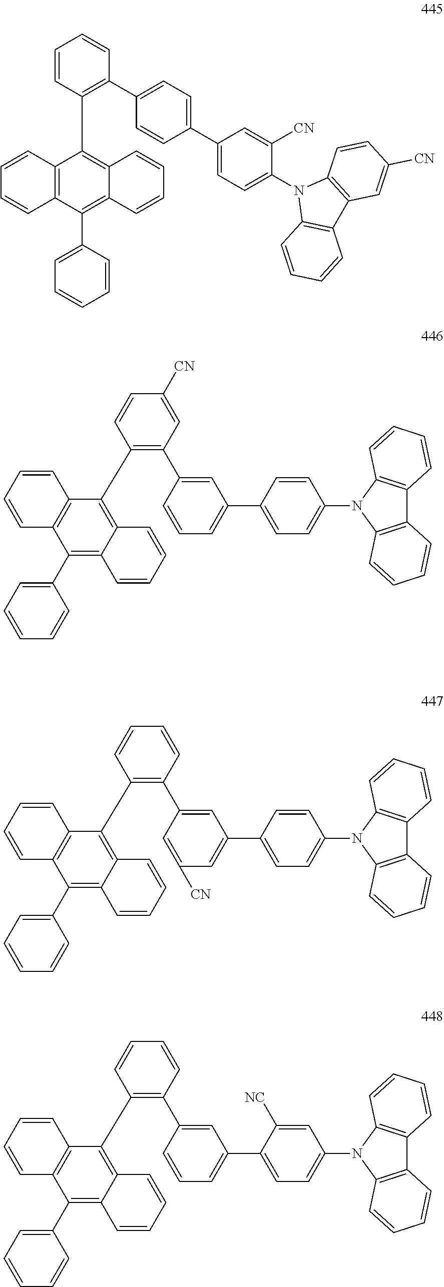 [SMILES: N#Cc1ccc2c(c1)c1ccccc1n2-c1ccc(-c2ccc(-c3ccccc3-c3c4ccccc4c(-c4ccccc4)c4ccccc34)cc2)cc1C#N ; N#Cc1ccc(-c2c3ccccc3c(-c3ccccc3)c3ccccc23)c(-c2cccc(-c3ccc(-n4c5ccccc5c5ccccc54)cc3)c2)c1 ; N#Cc1cc(-c2ccc(-n3c4ccccc4c4ccccc43)cc2)cc(-c2ccccc2-c2c3ccccc3c(-c3ccccc3)c3ccccc23)c1 ; N#Cc1cc(-n2c3ccccc3c3ccccc32)ccc1-c1cccc(-c2ccccc2-c2c3ccccc3c(-c3ccccc3)c3ccccc23)c1]

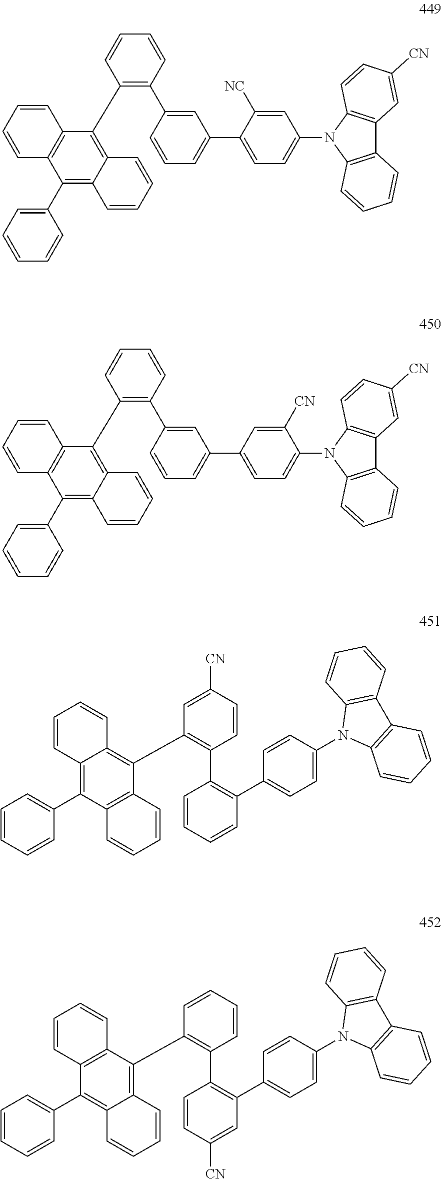 [SMILES: N#Cc1ccc2c(c1)c1ccccc1n2-c1ccc(-c2cccc(-c3ccccc3-c3c4ccccc4c(-c4ccccc4)c4ccccc34)c2)c(C#N)c1 ; N#Cc1ccc2c(c1)c1ccccc1n2-c1ccc(-c2cccc(-c3ccccc3-c3c4ccccc4c(-c4ccccc4)c4ccccc34)c2)cc1C#N ; N#Cc1ccc(-c2ccccc2-c2ccc(-n3c4ccccc4c4ccccc43)cc2)c(-c2c3ccccc3c(-c3ccccc3)c3ccccc23)c1 ; N#Cc1ccc(-c2ccccc2-c2c3ccccc3c(-c3ccccc3)c3ccccc23)c(-c2ccc(-n3c4ccccc4c4ccccc43)cc2)c1]

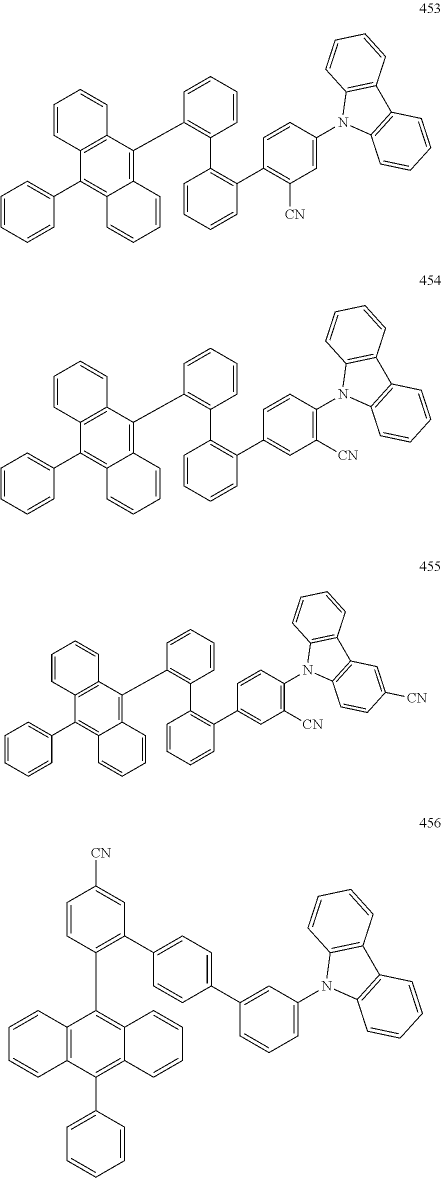 [SMILES: N#Cc1cc(-n2c3ccccc3c3ccccc32)ccc1-c1ccccc1-c1ccccc1-c1c2ccccc2c(-c2ccccc2)c2ccccc12 ; N#Cc1cc(-c2ccccc2-c2ccccc2-c2c3ccccc3c(-c3ccccc3)c3ccccc23)ccc1-n1c2ccccc2c2ccccc21 ; N#Cc1ccc2c(c1)c1ccccc1n2-c1ccc(-c2ccccc2-c2ccccc2-c2c3ccccc3c(-c3ccccc3)c3ccccc23)cc1C#N ; N#Cc1ccc(-c2c3ccccc3c(-c3ccccc3)c3ccccc23)c(-c2ccc(-c3cccc(-n4c5ccccc5c5ccccc54)c3)cc2)c1]

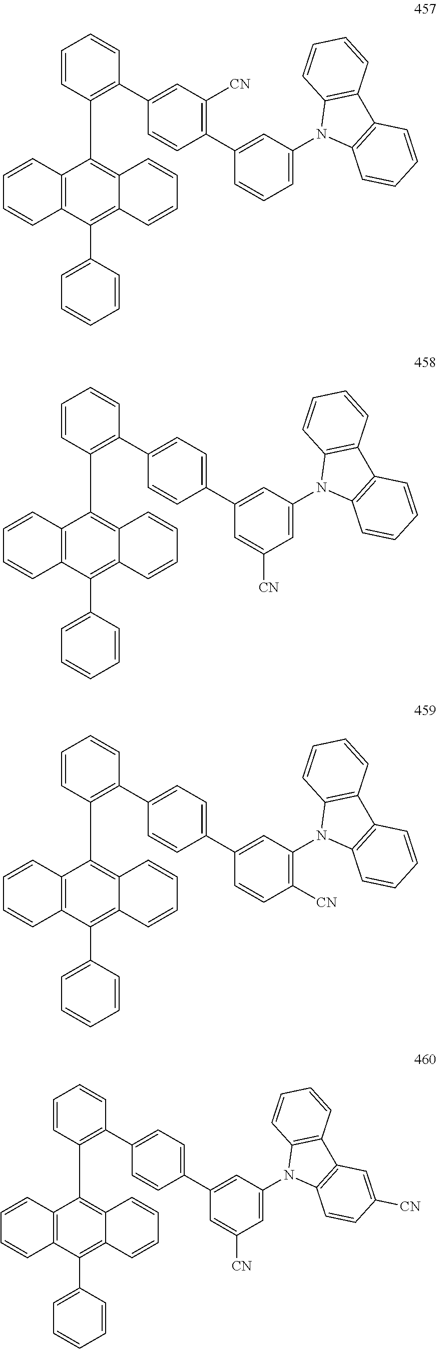 [SMILES: N#Cc1cc(-c2ccccc2-c2c3ccccc3c(-c3ccccc3)c3ccccc23)ccc1-c1cccc(-n2c3ccccc3c3ccccc32)c1 ; N#Cc1cc(-c2ccc(-c3ccccc3-c3c4ccccc4c(-c4ccccc4)c4ccccc34)cc2)cc(-n2c3ccccc3c3ccccc32)c1 ; N#Cc1ccc(-c2ccc(-c3ccccc3-c3c4ccccc4c(-c4ccccc4)c4ccccc34)cc2)cc1-n1c2ccccc2c2ccccc21 ; N#Cc1cc(-c2ccc(-c3ccccc3-c3c4ccccc4c(-c4ccccc4)c4ccccc34)cc2)cc(-n2c3ccccc3c3cc(C#N)ccc32)c1]

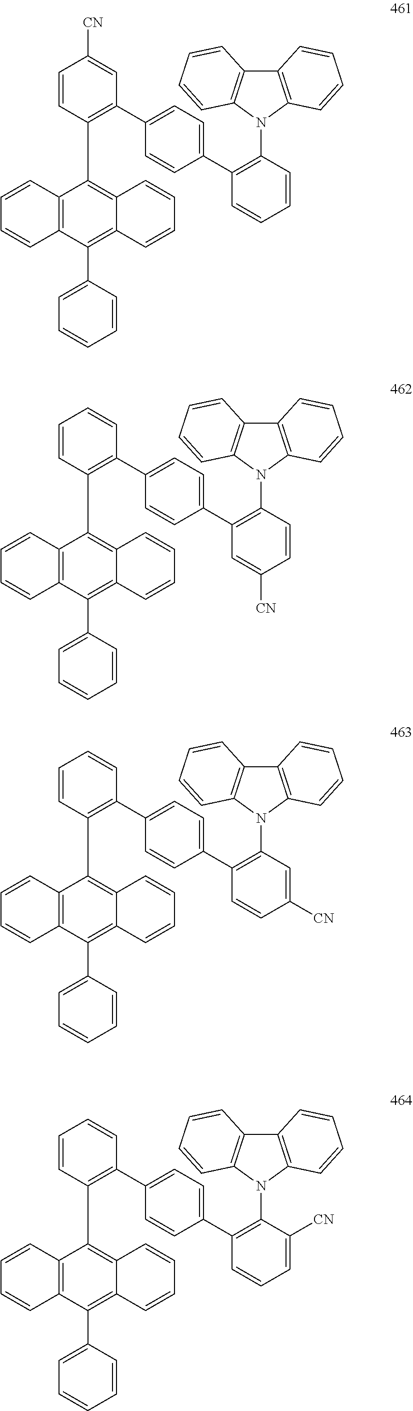 [SMILES: N#Cc1ccc(-c2c3ccccc3c(-c3ccccc3)c3ccccc23)c(-c2ccc(-c3ccccc3-n3c4ccccc4c4ccccc43)cc2)c1 ; N#Cc1ccc(-n2c3ccccc3c3ccccc32)c(-c2ccc(-c3ccccc3-c3c4ccccc4c(-c4ccccc4)c4ccccc34)cc2)c1 ; N#Cc1ccc(-c2ccc(-c3ccccc3-c3c4ccccc4c(-c4ccccc4)c4ccccc34)cc2)c(-n2c3ccccc3c3ccccc32)c1 ; N#Cc1cccc(-c2ccc(-c3ccccc3-c3c4ccccc4c(-c4ccccc4)c4ccccc34)cc2)c1-n1c2ccccc2c2ccccc21]

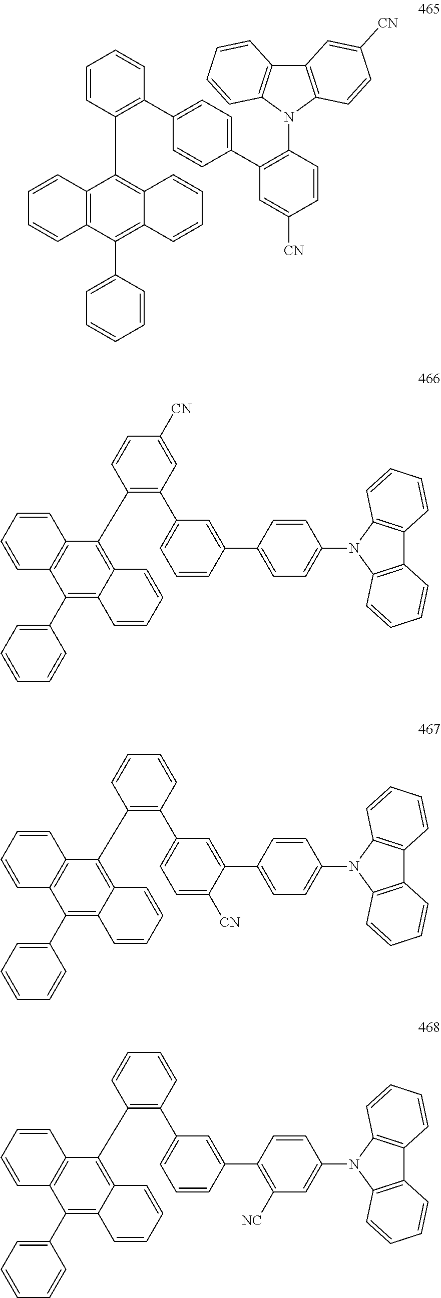 [SMILES: N#Cc1ccc(-n2c3ccccc3c3cc(C#N)ccc32)c(-c2ccc(-c3ccccc3-c3c4ccccc4c(-c4ccccc4)c4ccccc34)cc2)c1 ; N#Cc1ccc(-c2c3ccccc3c(-c3ccccc3)c3ccccc23)c(-c2cccc(-c3ccc(-n4c5ccccc5c5ccccc54)cc3)c2)c1 ; N#Cc1ccc(-c2ccccc2-c2c3ccccc3c(-c3ccccc3)c3ccccc23)cc1-c1ccc(-n2c3ccccc3c3ccccc32)cc1 ; N#Cc1cc(-n2c3ccccc3c3ccccc32)ccc1-c1cccc(-c2ccccc2-c2c3ccccc3c(-c3ccccc3)c3ccccc23)c1]

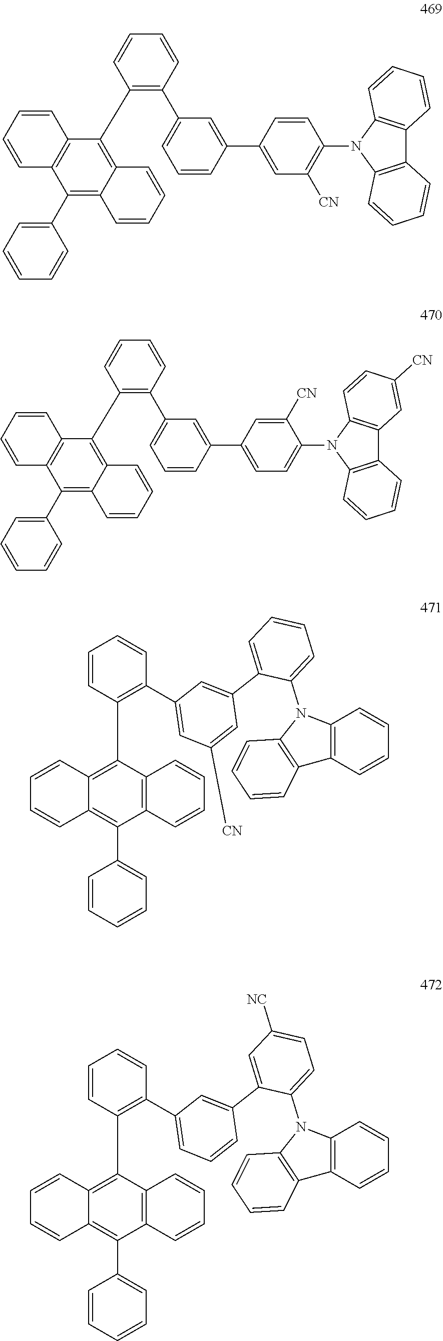 [SMILES: N#Cc1cc(-c2cccc(-c3ccccc3-c3c4ccccc4c(-c4ccccc4)c4ccccc34)c2)ccc1-n1c2ccccc2c2ccccc21 ; N#Cc1ccc2c(c1)c1ccccc1n2-c1ccc(-c2cccc(-c3ccccc3-c3c4ccccc4c(-c4ccccc4)c4ccccc34)c2)cc1C#N ; N#Cc1cc(-c2ccccc2-c2c3ccccc3c(-c3ccccc3)c3ccccc23)cc(-c2ccccc2-n2c3ccccc3c3ccccc32)c1 ; N#Cc1ccc(-n2c3ccccc3c3ccccc32)c(-c2cccc(-c3ccccc3-c3c4ccccc4c(-c4ccccc4)c4ccccc34)c2)c1]

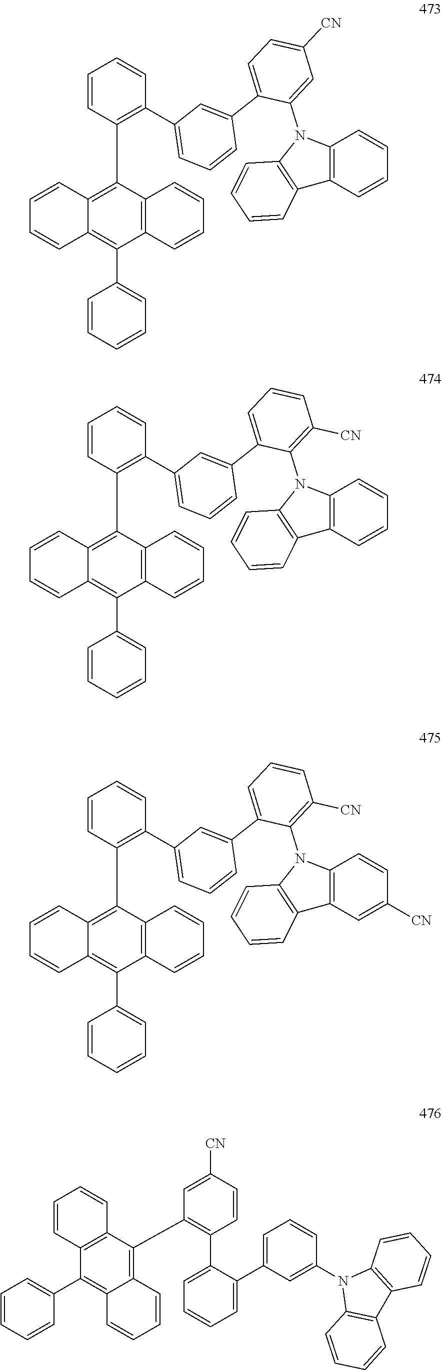 [SMILES: N#Cc1ccc(-c2cccc(-c3ccccc3-c3c4ccccc4c(-c4ccccc4)c4ccccc34)c2)c(-n2c3ccccc3c3ccccc32)c1 ; N#Cc1cccc(-c2cccc(-c3ccccc3-c3c4ccccc4c(-c4ccccc4)c4ccccc34)c2)c1-n1c2ccccc2c2ccccc21 ; N#Cc1ccc2c(c1)c1ccccc1n2-c1c(C#N)cccc1-c1cccc(-c2ccccc2-c2c3ccccc3c(-c3ccccc3)c3ccccc23)c1 ; N#Cc1ccc(-c2ccccc2-c2cccc(-n3c4ccccc4c4ccccc43)c2)c(-c2c3ccccc3c(-c3ccccc3)c3ccccc23)c1]

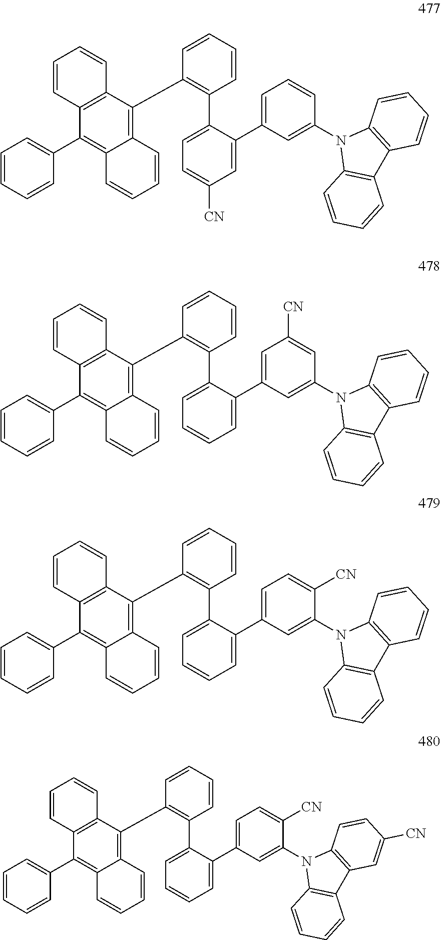 [SMILES: N#Cc1ccc(-c2ccccc2-c2c3ccccc3c(-c3ccccc3)c3ccccc23)c(-c2cccc(-n3c4ccccc4c4ccccc43)c2)c1 ; N#Cc1cc(-c2ccccc2-c2ccccc2-c2c3ccccc3c(-c3ccccc3)c3ccccc23)cc(-n2c3ccccc3c3ccccc32)c1 ; N#Cc1ccc(-c2ccccc2-c2ccccc2-c2c3ccccc3c(-c3ccccc3)c3ccccc23)cc1-n1c2ccccc2c2ccccc21 ; N#Cc1ccc2c(c1)c1ccccc1n2-c1cc(-c2ccccc2-c2ccccc2-c2c3ccccc3c(-c3ccccc3)c3ccccc23)ccc1C#N]

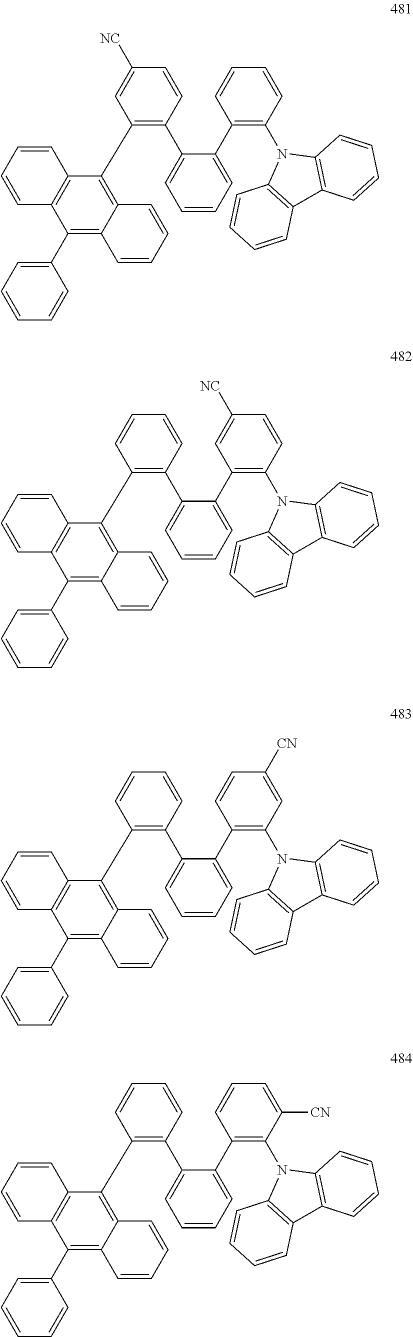 [SMILES: N#Cc1ccc(-c2ccccc2-c2ccccc2-n2c3ccccc3c3ccccc32)c(-c2c3ccccc3c(-c3ccccc3)c3ccccc23)c1 ; N#Cc1ccc(-n2c3ccccc3c3ccccc32)c(-c2ccccc2-c2ccccc2-c2c3ccccc3c(-c3ccccc3)c3ccccc23)c1 ; N#Cc1ccc(-c2ccccc2-c2ccccc2-c2c3ccccc3c(-c3ccccc3)c3ccccc23)c(-n2c3ccccc3c3ccccc32)c1 ; N#Cc1cccc(-c2ccccc2-c2ccccc2-c2c3ccccc3c(-c3ccccc3)c3ccccc23)c1-n1c2ccccc2c2ccccc21]

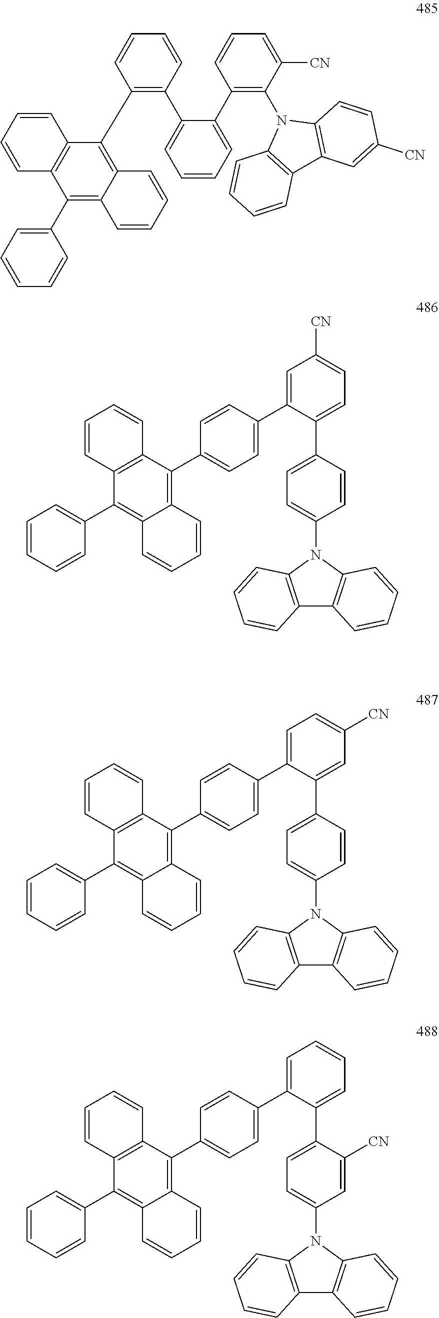 [SMILES: N#Cc1ccc2c(c1)c1ccccc1n2-c1c(C#N)cccc1-c1ccccc1-c1ccccc1-c1c2ccccc2c(-c2ccccc2)c2ccccc12 ; N#Cc1ccc(-c2ccc(-n3c4ccccc4c4ccccc43)cc2)c(-c2ccc(-c3c4ccccc4c(-c4ccccc4)c4ccccc34)cc2)c1 ; N#Cc1ccc(-c2ccc(-c3c4ccccc4c(-c4ccccc4)c4ccccc34)cc2)c(-c2ccc(-n3c4ccccc4c4ccccc43)cc2)c1 ; N#Cc1cc(-n2c3ccccc3c3ccccc32)ccc1-c1ccccc1-c1ccc(-c2c3ccccc3c(-c3ccccc3)c3ccccc23)cc1]

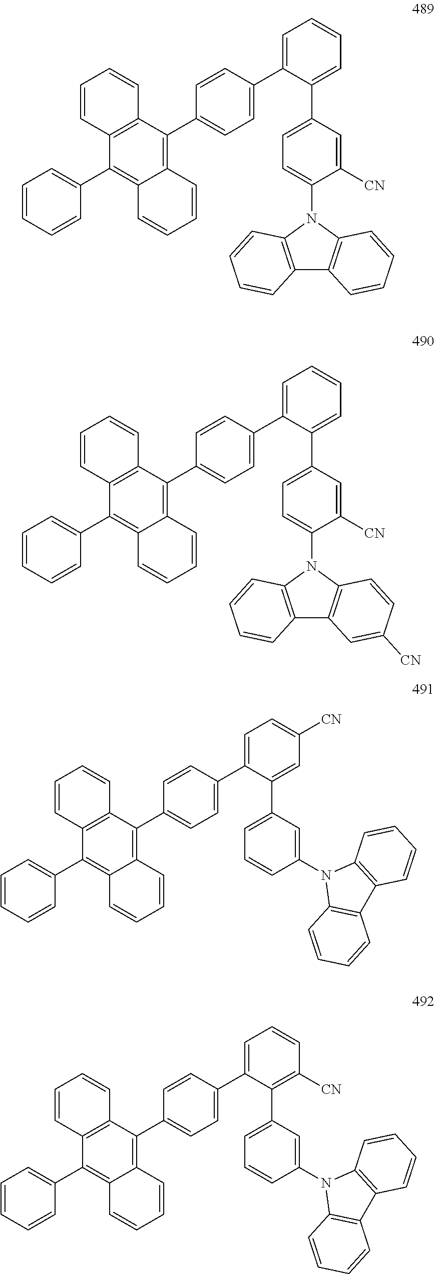 [SMILES: N#Cc1cc(-c2ccccc2-c2ccc(-c3c4ccccc4c(-c4ccccc4)c4ccccc34)cc2)ccc1-n1c2ccccc2c2ccccc21 ; N#Cc1ccc2c(c1)c1ccccc1n2-c1ccc(-c2ccccc2-c2ccc(-c3c4ccccc4c(-c4ccccc4)c4ccccc34)cc2)cc1C#N ; N#Cc1ccc(-c2ccc(-c3c4ccccc4c(-c4ccccc4)c4ccccc34)cc2)c(-c2cccc(-n3c4ccccc4c4ccccc43)c2)c1 ; N#Cc1cccc(-c2ccc(-c3c4ccccc4c(-c4ccccc4)c4ccccc34)cc2)c1-c1cccc(-n2c3ccccc3c3ccccc32)c1]

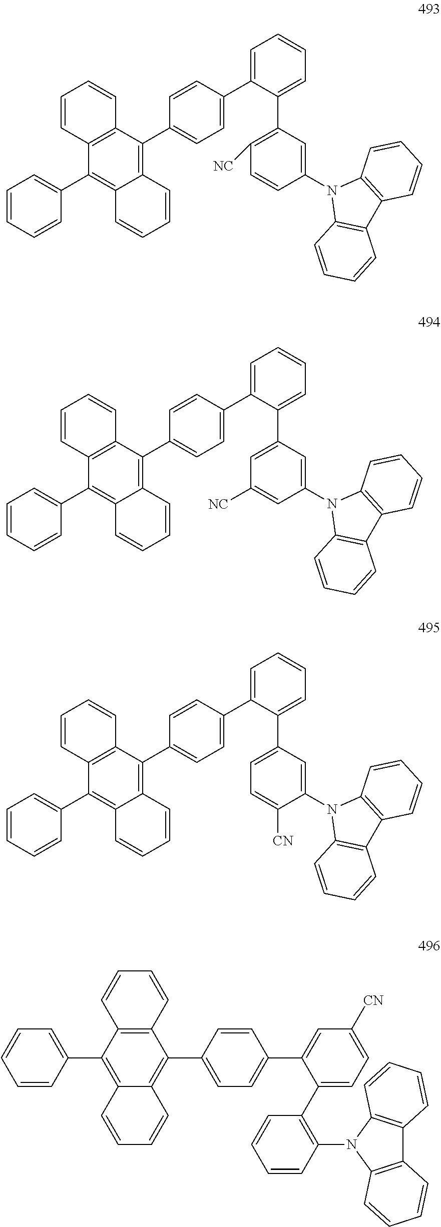 [SMILES: N#Cc1ccc(-n2c3ccccc3c3ccccc32)cc1-c1ccccc1-c1ccc(-c2c3ccccc3c(-c3ccccc3)c3ccccc23)cc1 ; N#Cc1cc(-c2ccccc2-c2ccc(-c3c4ccccc4c(-c4ccccc4)c4ccccc34)cc2)cc(-n2c3ccccc3c3ccccc32)c1 ; N#Cc1ccc(-c2ccccc2-c2ccc(-c3c4ccccc4c(-c4ccccc4)c4ccccc34)cc2)cc1-n1c2ccccc2c2ccccc21 ; N#Cc1ccc(-c2ccccc2-n2c3ccccc3c3ccccc32)c(-c2ccc(-c3c4ccccc4c(-c4ccccc4)c4ccccc34)cc2)c1]

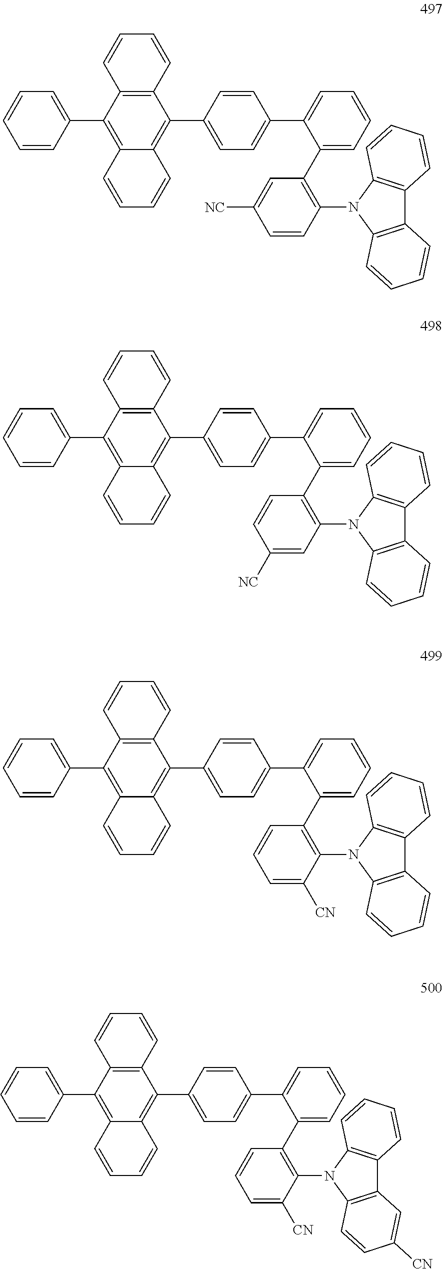 [SMILES: N#Cc1ccc(-n2c3ccccc3c3ccccc32)c(-c2ccccc2-c2ccc(-c3c4ccccc4c(-c4ccccc4)c4ccccc34)cc2)c1 ; N#Cc1ccc(-c2ccccc2-c2ccc(-c3c4ccccc4c(-c4ccccc4)c4ccccc34)cc2)c(-n2c3ccccc3c3ccccc32)c1 ; N#Cc1cccc(-c2ccccc2-c2ccc(-c3c4ccccc4c(-c4ccccc4)c4ccccc34)cc2)c1-n1c2ccccc2c2ccccc21 ; N#Cc1ccc2c(c1)c1ccccc1n2-c1c(C#N)cccc1-c1ccccc1-c1ccc(-c2c3ccccc3c(-c3ccccc3)c3ccccc23)cc1]

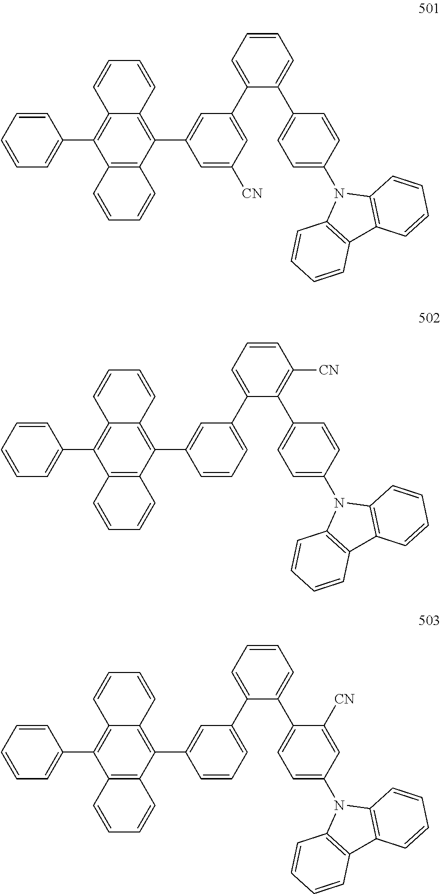 [SMILES: N#Cc1cc(-c2ccccc2-c2ccc(-n3c4ccccc4c4ccccc43)cc2)cc(-c2c3ccccc3c(-c3ccccc3)c3ccccc23)c1 ; N#Cc1cccc(-c2cccc(-c3c4ccccc4c(-c4ccccc4)c4ccccc34)c2)c1-c1ccc(-n2c3ccccc3c3ccccc32)cc1 ; N#Cc1cc(-n2c3ccccc3c3ccccc32)ccc1-c1ccccc1-c1cccc(-c2c3ccccc3c(-c3ccccc3)c3ccccc23)c1]

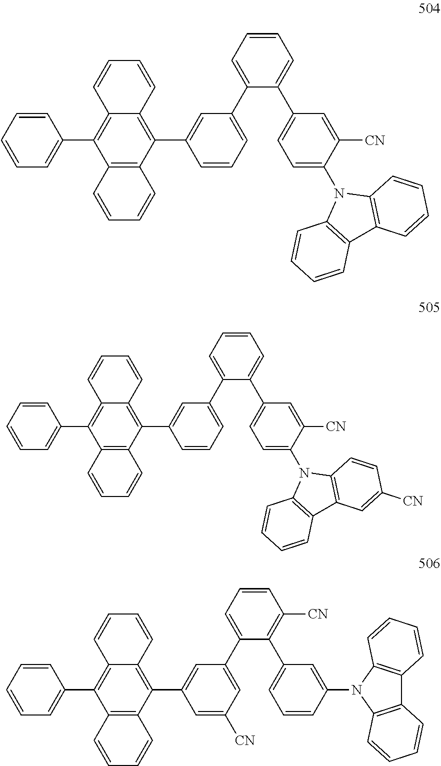

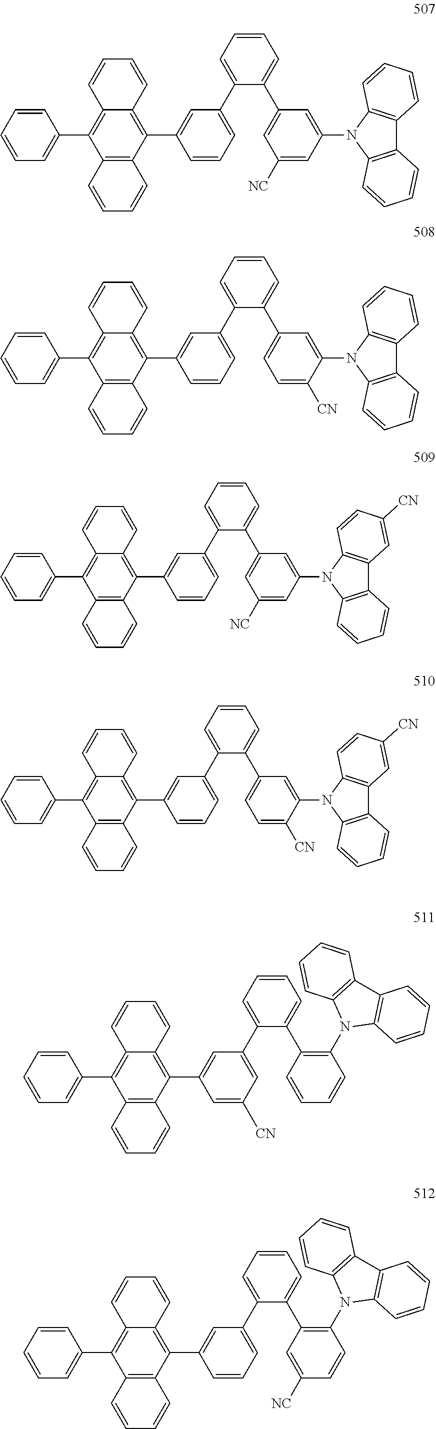

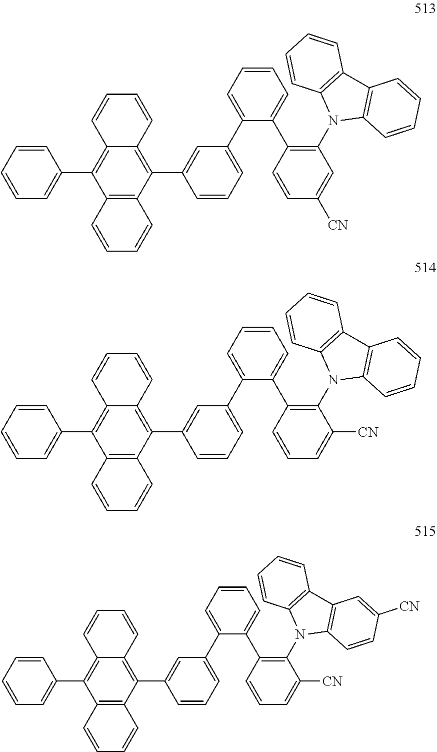 [SMILES: N#Cc1ccc(-c2ccccc2-c2cccc(-c3c4ccccc4c(-c4ccccc4)c4ccccc34)c2)c(-n2c3ccccc3c3ccccc32)c1 ; N#Cc1cccc(-c2ccccc2-c2cccc(-c3c4ccccc4c(-c4ccccc4)c4ccccc34)c2)c1-n1c2ccccc2c2ccccc21 ; N#Cc1ccc2c(c1)c1ccccc1n2-c1c(C#N)cccc1-c1ccccc1-c1cccc(-c2c3ccccc3c(-c3ccccc3)c3ccccc23)c1]

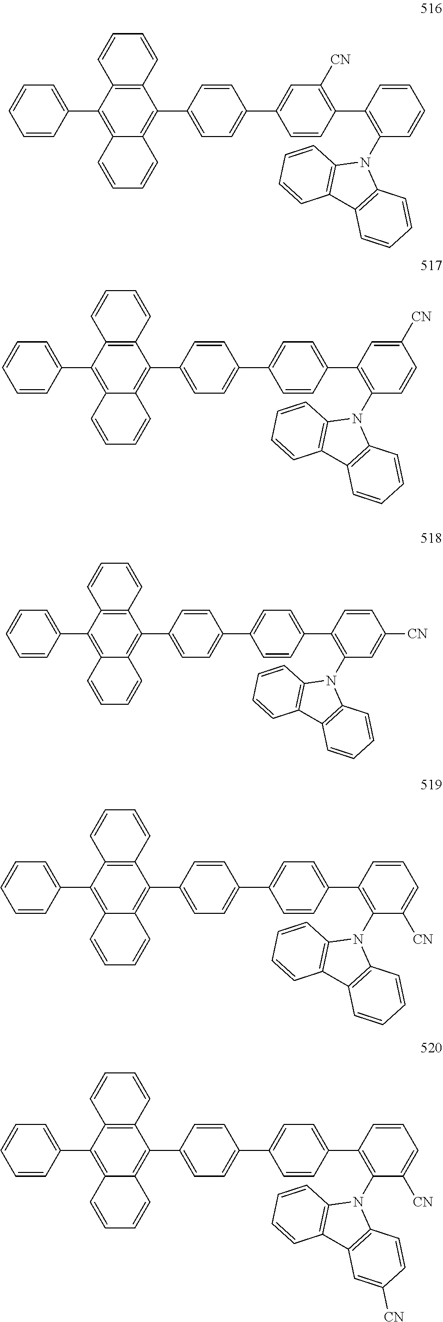 [SMILES: N#Cc1cc(-c2ccc(-c3c4ccccc4c(-c4ccccc4)c4ccccc34)cc2)ccc1-c1ccccc1-n1c2ccccc2c2ccccc21 ; N#Cc1ccc(-n2c3ccccc3c3ccccc32)c(-c2ccc(-c3ccc(-c4c5ccccc5c(-c5ccccc5)c5ccccc45)cc3)cc2)c1 ; N#Cc1ccc(-c2ccc(-c3ccc(-c4c5ccccc5c(-c5ccccc5)c5ccccc45)cc3)cc2)c(-n2c3ccccc3c3ccccc32)c1 ; N#Cc1cccc(-c2ccc(-c3ccc(-c4c5ccccc5c(-c5ccccc5)c5ccccc45)cc3)cc2)c1-n1c2ccccc2c2ccccc21 ; N#Cc1ccc2c(c1)c1ccccc1n2-c1c(C#N)cccc1-c1ccc(-c2ccc(-c3c4ccccc4c(-c4ccccc4)c4ccccc34)cc2)cc1]

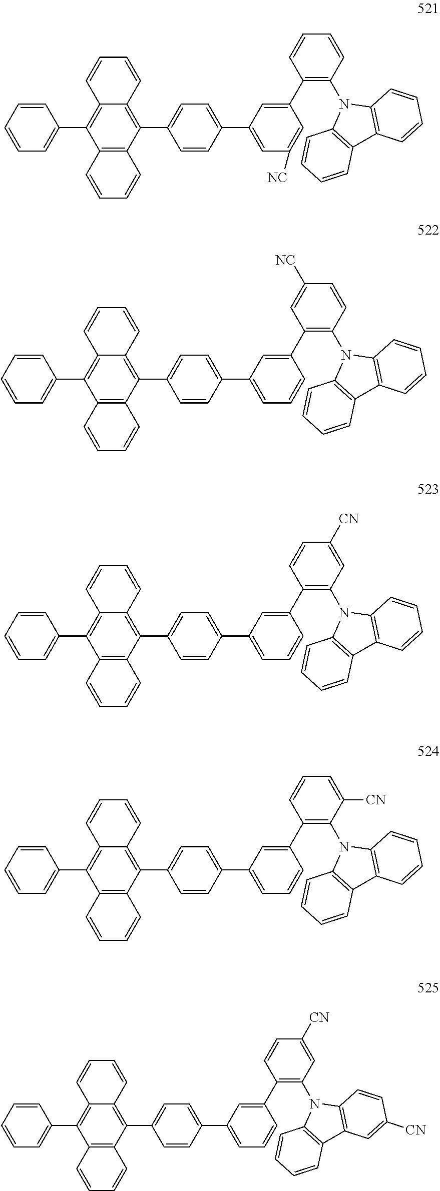 [SMILES: N#Cc1cc(-c2ccc(-c3c4ccccc4c(-c4ccccc4)c4ccccc34)cc2)cc(-c2ccccc2-n2c3ccccc3c3ccccc32)c1 ; N#Cc1ccc(-n2c3ccccc3c3ccccc32)c(-c2cccc(-c3ccc(-c4c5ccccc5c(-c5ccccc5)c5ccccc45)cc3)c2)c1 ; N#Cc1ccc(-c2cccc(-c3ccc(-c4c5ccccc5c(-c5ccccc5)c5ccccc45)cc3)c2)c(-n2c3ccccc3c3ccccc32)c1 ; N#Cc1cccc(-c2cccc(-c3ccc(-c4c5ccccc5c(-c5ccccc5)c5ccccc45)cc3)c2)c1-n1c2ccccc2c2ccccc21 ; N#Cc1ccc(-c2cccc(-c3ccc(-c4c5ccccc5c(-c5ccccc5)c5ccccc45)cc3)c2)c(-n2c3ccccc3c3cc(C#N)ccc32)c1]

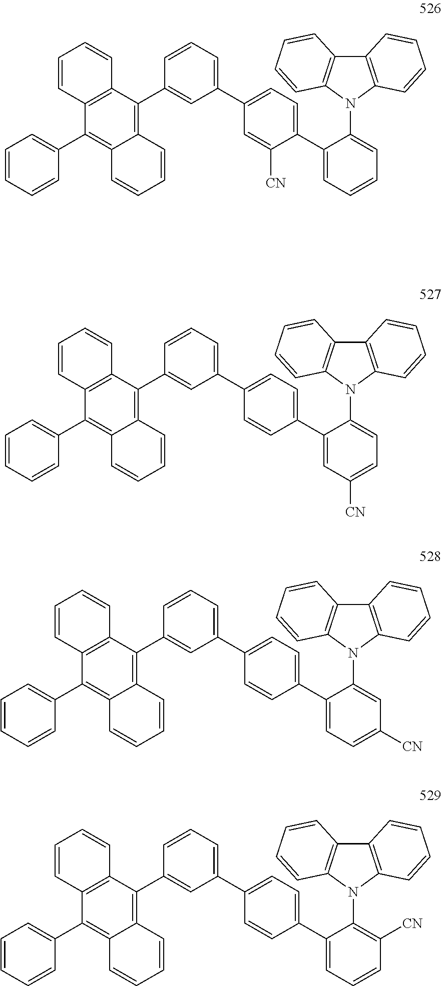 [SMILES: N#Cc1cc(-c2cccc(-c3c4ccccc4c(-c4ccccc4)c4ccccc34)c2)ccc1-c1ccccc1-n1c2ccccc2c2ccccc21 ; N#Cc1ccc(-n2c3ccccc3c3ccccc32)c(-c2ccc(-c3cccc(-c4c5ccccc5c(-c5ccccc5)c5ccccc45)c3)cc2)c1 ; N#Cc1ccc(-c2ccc(-c3cccc(-c4c5ccccc5c(-c5ccccc5)c5ccccc45)c3)cc2)c(-n2c3ccccc3c3ccccc32)c1 ; N#Cc1cccc(-c2ccc(-c3cccc(-c4c5ccccc5c(-c5ccccc5)c5ccccc45)c3)cc2)c1-n1c2ccccc2c2ccccc21]

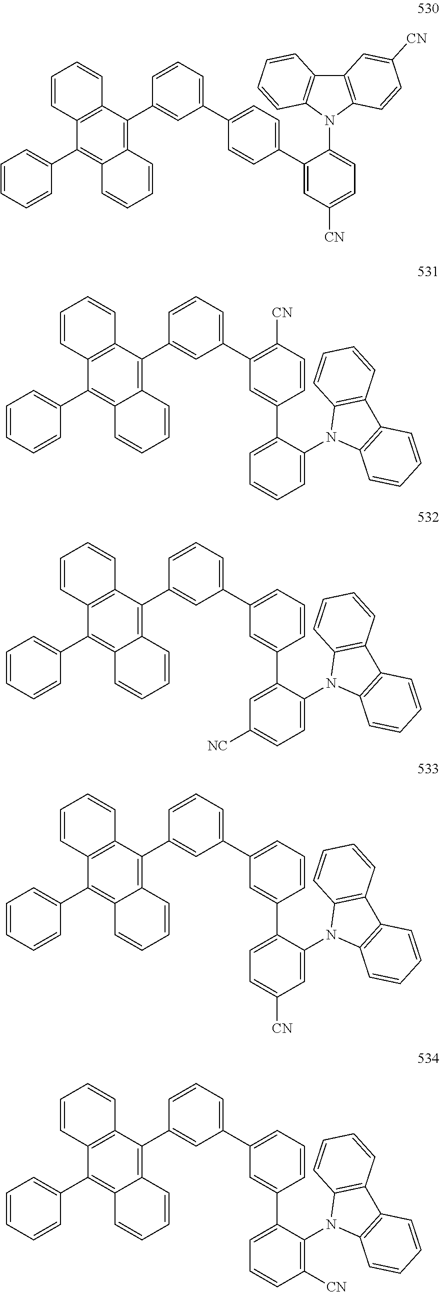 [SMILES: N#Cc1ccc(-n2c3ccccc3c3cc(C#N)ccc32)c(-c2ccc(-c3cccc(-c4c5ccccc5c(-c5ccccc5)c5ccccc45)c3)cc2)c1 ; N#Cc1ccc(-c2ccccc2-n2c3ccccc3c3ccccc32)cc1-c1cccc(-c2c3ccccc3c(-c3ccccc3)c3ccccc23)c1 ; N#Cc1ccc(-n2c3ccccc3c3ccccc32)c(-c2cccc(-c3cccc(-c4c5ccccc5c(-c5ccccc5)c5ccccc45)c3)c2)c1 ; N#Cc1ccc(-c2cccc(-c3cccc(-c4c5ccccc5c(-c5ccccc5)c5ccccc45)c3)c2)c(-n2c3ccccc3c3ccccc32)c1 ; N#Cc1cccc(-c2cccc(-c3cccc(-c4c5ccccc5c(-c5ccccc5)c5ccccc45)c3)c2)c1-n1c2ccccc2c2ccccc21]

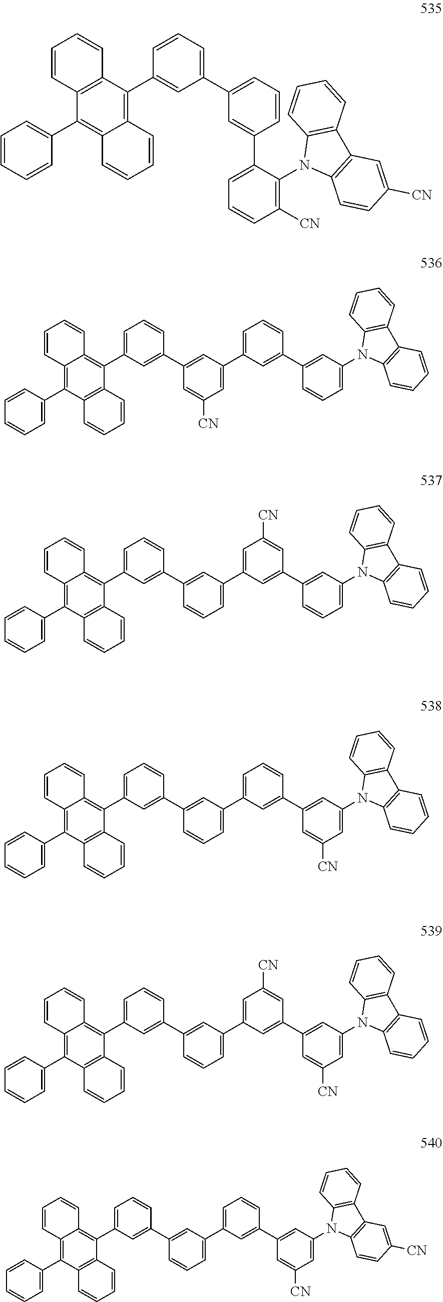

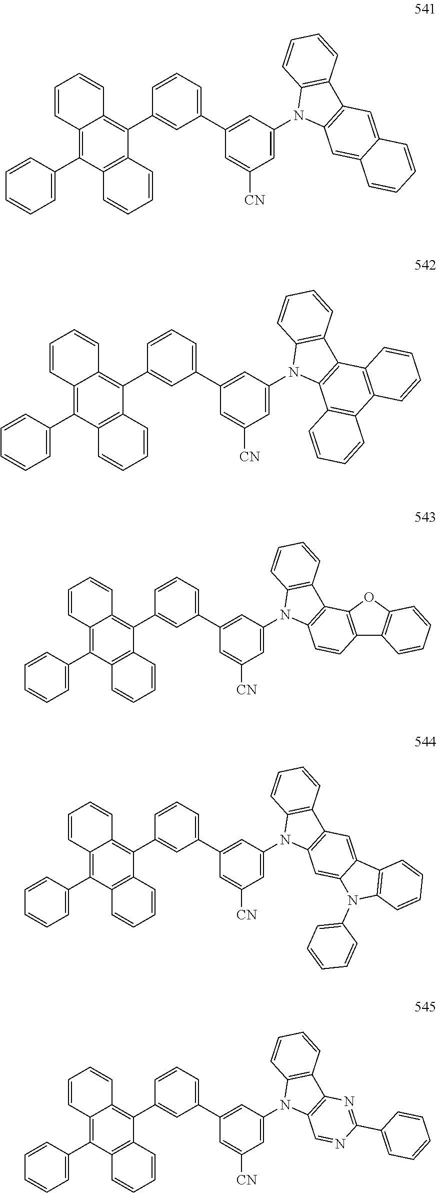 [SMILES: N#Cc1cc(-c2cccc(-c3c4ccccc4c(-c4ccccc4)c4ccccc34)c2)cc(-n2c3ccccc3c3cc4ccccc4cc32)c1 ; N#Cc1cc(-c2cccc(-c3c4ccccc4c(-c4ccccc4)c4ccccc34)c2)cc(-n2c3ccccc3c3c4ccccc4c4ccccc4c32)c1 ; N#Cc1cc(-c2cccc(-c3c4ccccc4c(-c4ccccc4)c4ccccc34)c2)cc(-n2c3ccccc3c3c4oc5ccccc5c4ccc32)c1 ; N#Cc1cc(-c2cccc(-c3c4ccccc4c(-c4ccccc4)c4ccccc34)c2)cc(-n2c3ccccc3c3cc4c5ccccc5n(-c5ccccc5)c4cc32)c1 ; N#Cc1cc(-c2cccc(-c3c4ccccc4c(-c4ccccc4)c4ccccc34)c2)cc(-n2c3ccccc3c3nc(-c4ccccc4)ncc32)c1]

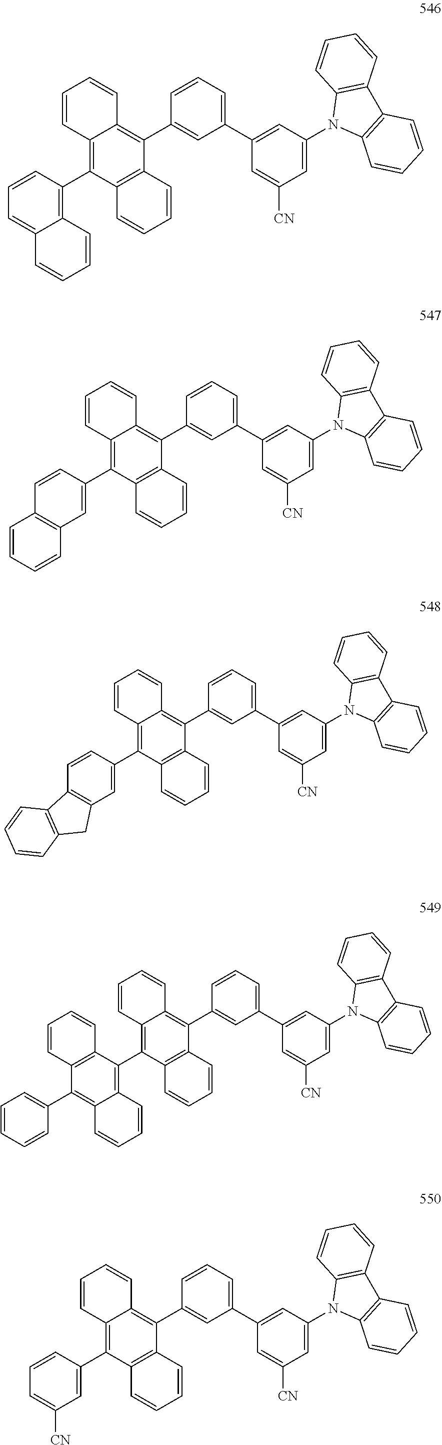 [SMILES: N#Cc1cc(-c2cccc(-c3c4ccccc4c(-c4cccc5ccccc45)c4ccccc34)c2)cc(-n2c3ccccc3c3ccccc32)c1 ; N#Cc1cc(-c2cccc(-c3c4ccccc4c(-c4ccc5ccccc5c4)c4ccccc34)c2)cc(-n2c3ccccc3c3ccccc32)c1 ; N#Cc1cc(-c2cccc(-c3c4ccccc4c(-c4ccc5c(c4)Cc4ccccc4-5)c4ccccc34)c2)cc(-n2c3ccccc3c3ccccc32)c1 ; N#Cc1cc(-c2cccc(-c3c4ccccc4c(-c4c5ccccc5c(-c5ccccc5)c5ccccc45)c4ccccc34)c2)cc(-n2c3ccccc3c3ccccc32)c1 ; N#Cc1cccc(-c2c3ccccc3c(-c3cccc(-c4cc(C#N)cc(-n5c6ccccc6c6ccccc65)c4)c3)c3ccccc23)c1]

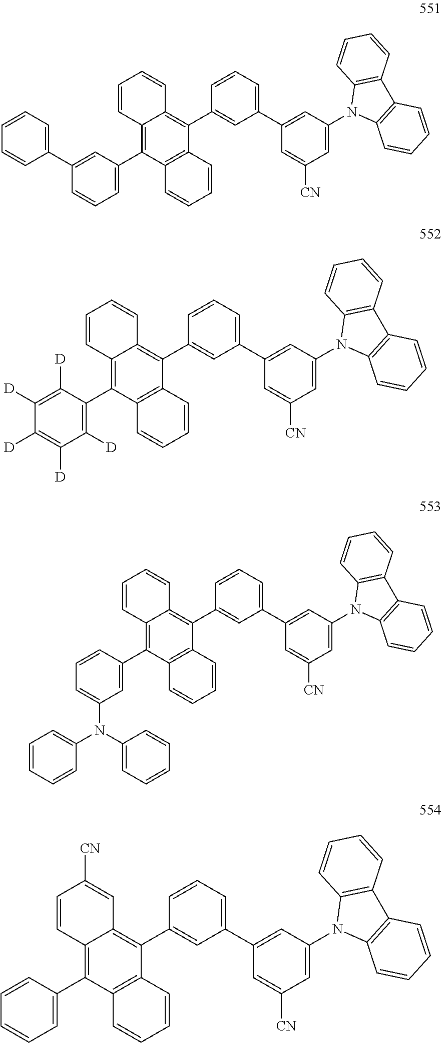 [SMILES: N#Cc1cc(-c2cccc(-c3c4ccccc4c(-c4cccc(-c5ccccc5)c4)c4ccccc34)c2)cc(-n2c3ccccc3c3ccccc32)c1 ; [2H]c1c([2H])c([2H])c(-c2c3ccccc3c(-c3cccc(-c4cc(C#N)cc(-n5c6ccccc6c6ccccc65)c4)c3)c3ccccc23)c([2H])c1[2H] ; N#Cc1cc(-c2cccc(-c3c4ccccc4c(-c4cccc(N(c5ccccc5)c5ccccc5)c4)c4ccccc34)c2)cc(-n2c3ccccc3c3ccccc32)c1 ; N#Cc1cc(-c2cccc(-c3c4ccccc4c(-c4ccccc4)c4ccc(C#N)cc34)c2)cc(-n2c3ccccc3c3ccccc32)c1]

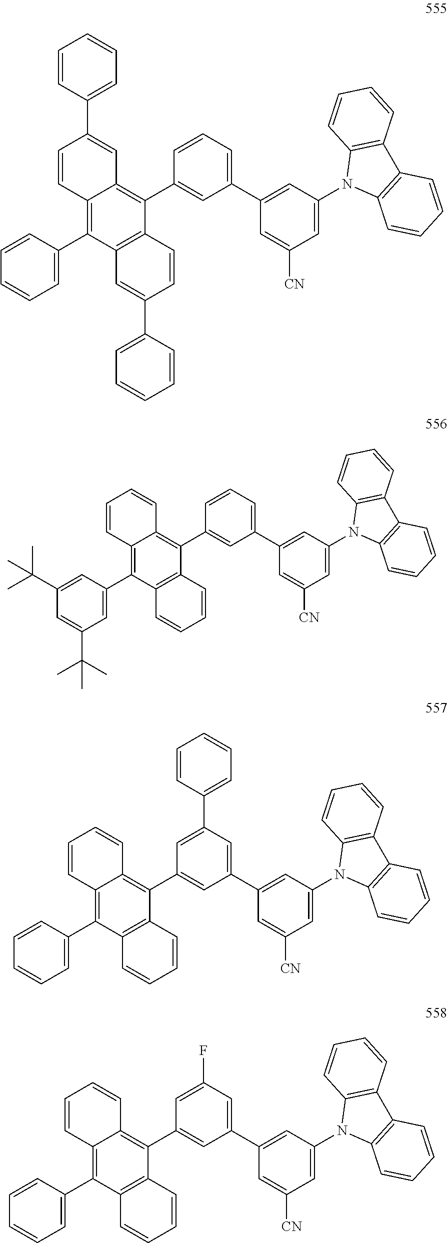 [SMILES: N#Cc1cc(-c2cccc(-c3c4ccc(-c5ccccc5)cc4c(-c4ccccc4)c4ccc(-c5ccccc5)cc34)c2)cc(-n2c3ccccc3c3ccccc32)c1 ; CC(C)(C)c1cc(-c2c3ccccc3c(-c3cccc(-c4cc(C#N)cc(-n5c6ccccc6c6ccccc65)c4)c3)c3ccccc23)cc(C(C)(C)C)c1 ; N#Cc1cc(-c2cc(-c3ccccc3)cc(-c3c4ccccc4c(-c4ccccc4)c4ccccc34)c2)cc(-n2c3ccccc3c3ccccc32)c1 ; N#Cc1cc(-c2cc(F)cc(-c3c4ccccc4c(-c4ccccc4)c4ccccc34)c2)cc(-n2c3ccccc3c3ccccc32)c1]

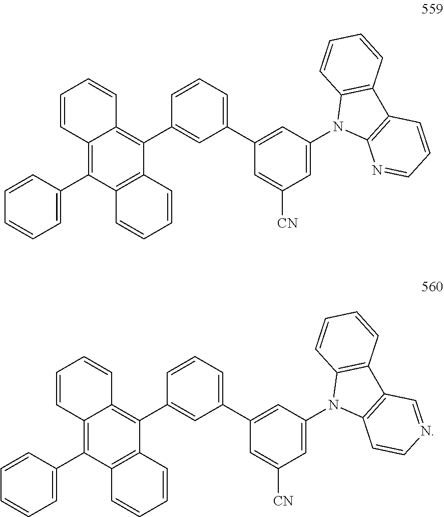 [SMILES: N#Cc1cc(-c2cccc(-c3c4ccccc4c(-c4ccccc4)c4ccccc34)c2)cc(-n2c3ccccc3c3cccnc32)c1 ; N#Cc1cc(-c2cccc(-c3c4ccccc4c(-c4ccccc4)c4ccccc34)c2)cc(-n2c3ccccc3c3cnccc32)c1]

In an embodiment, the fluorescent host may be represented by Formula FH-4:

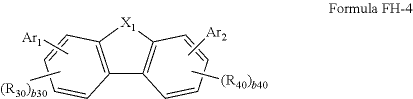

wherein, in Formula FH-4,
X₁ may be O or Se,
Ar₁ may be a group represented by Formula 1A, and Ar₂ may be a group represented by Formula 1B:

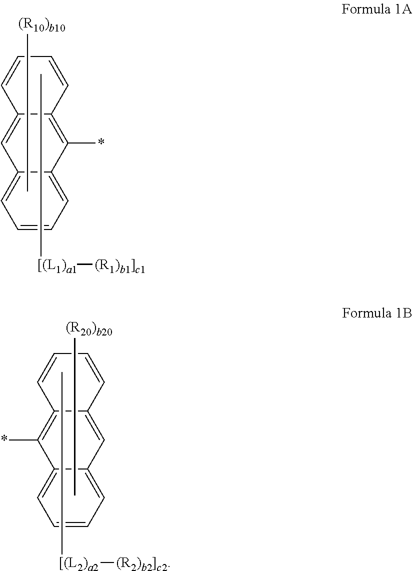

In Formulae 1A and 1B,
L₁ and L₂ may each independently a substituted or unsubstituted C₅-C₃₀ carbocyclic group or a substituted or unsubstituted C₁-C₃₀ heterocyclic group,
a1 and a2 may each independently be an integer from 0 to 3,
when a1 is 2 or greater, two or more of L₁ may be identical to or different from each other, and when a2 is 2 or greater, two or more of L₂ may be identical to or different from each other,
R₁, R₂, R₁₀, R₂₀, R₃₀, and R₄₀ may each independently be hydrogen, deuterium, —F, —Cl, —Br, —I, —SF₅, a hydroxyl group, a cyano group, a nitro group, an amidino group, a hydrazine group, a hydrazone group, a carboxylic acid group or a salt thereof, a sulfonic acid group or a salt thereof, a phosphoric acid group or a salt thereof, a substituted or unsubstituted C₁-C₆₀ alkyl group, a substituted or unsubstituted C₂-C₆₀ alkenyl group, a substituted or unsubstituted C₂-C₆₀ alkynyl group, a substituted or unsubstituted C₁-C₆₀ alkoxy group, a substituted or unsubstituted C₃-C₁₀ cycloalkyl group, a substituted or unsubstituted C₁-C₁₀ heterocycloalkyl group, a substituted or unsubstituted C₃-C₁₀ cycloalkenyl group, a substituted or unsubstituted C₂-C₁₀ heterocycloalkenyl group, a substituted or unsubstituted C₆-C₆₀ aryl group, a substituted or unsubstituted C₆-C₆₀ aryloxy group, a substituted or unsubstituted C₆-C₆₀ arylthio group, a substituted or unsubstituted C₁-C₆₀ heteroaryl group, a substituted or unsubstituted monovalent non-aromatic condensed polycyclic group, a substituted or unsubstituted monovalent non-aromatic condensed heteropolycyclic group, —N(Q₁)(Q₂), —Si(Q₃)(Q₄)(Q₅), —B(Q₆)(Q₇), or —P(═O)(Q₈)(Q₉),
b1 and b2 may each independently be an integer from 1 to 5,
when b1 is 2 or greater, two or more of R₁ may be identical to or different from each other, and when b2 is 2 or greater, two or more of R₂ may be identical to or different from each other,
b10 and b20 may each independently an integer from 1 to 8,
b30 and b40 may each independently an integer from 1 to 3,
c1 and c2 may each independently be an integer from 1 to 8, and
the sum of b10 and c1 may be 9, and the sum of b20 and c2 may be 9.

In an embodiment, the fluorescent host represented by Formula FH-4 may be a compound of Group FH4, but embodiments of the present disclosure are not limited thereto:

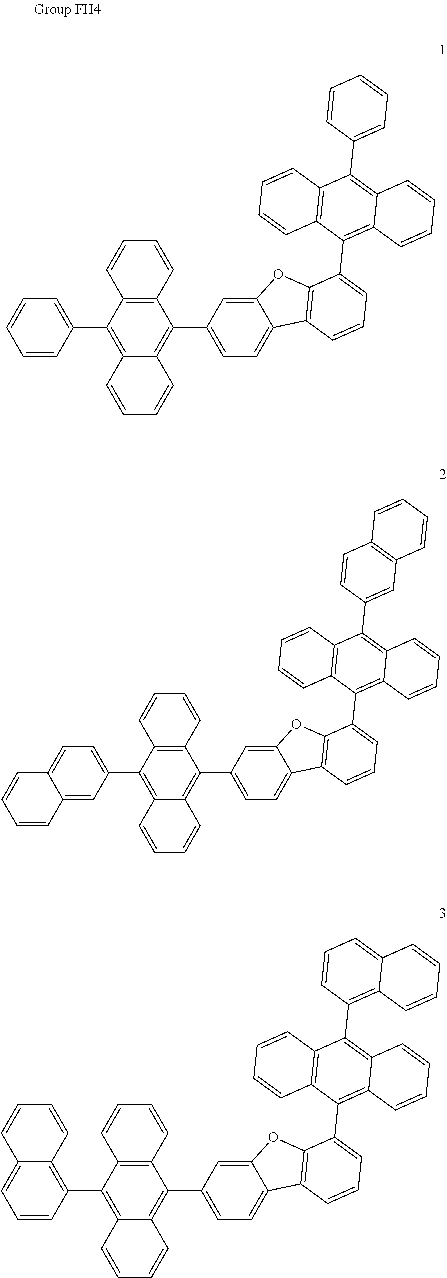 [SMILES: c1ccc(-c2c3ccccc3c(-c3ccc4c(c3)oc3c(-c5c6ccccc6c(-c6ccccc6)c6ccccc56)cccc34)c3ccccc23)cc1 ; c1ccc2cc(-c3c4ccccc4c(-c4ccc5c(c4)oc4c(-c6c7ccccc7c(-c7ccc8ccccc8c7)c7ccccc67)cccc45)c4ccccc34)ccc2c1 ; c1ccc2c(-c3c4ccccc4c(-c4ccc5c(c4)oc4c(-c6c7ccccc7c(-c7cccc8ccccc78)c7ccccc67)cccc45)c4ccccc34)cccc2c1]

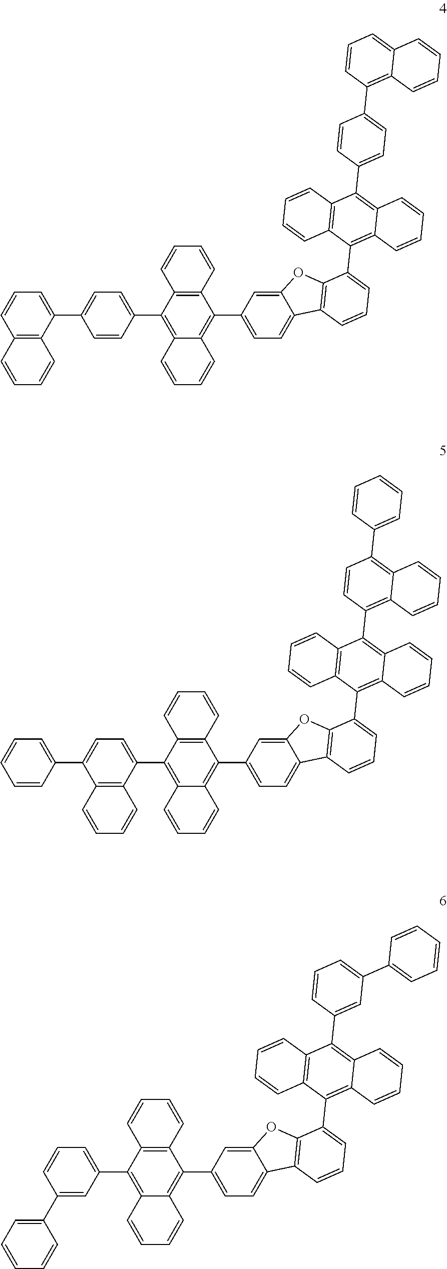 [SMILES: c1ccc2c(-c3ccc(-c4c5ccccc5c(-c5ccc6c(c5)oc5c(-c7c8ccccc8c(-c8ccc(-c9cccc%10ccccc9%10)cc8)c8ccccc78)cccc56)c5ccccc45)cc3)cccc2c1 ; c1ccc(-c2ccc(-c3c4ccccc4c(-c4ccc5c(c4)oc4c(-c6c7ccccc7c(-c7ccc(-c8ccccc8)c8ccccc78)c7ccccc67)cccc45)c4ccccc34)c3ccccc23)cc1 ; c1ccc(-c2cccc(-c3c4ccccc4c(-c4ccc5c(c4)oc4c(-c6c7ccccc7c(-c7cccc(-c8ccccc8)c7)c7ccccc67)cccc45)c4ccccc34)c2)cc1]

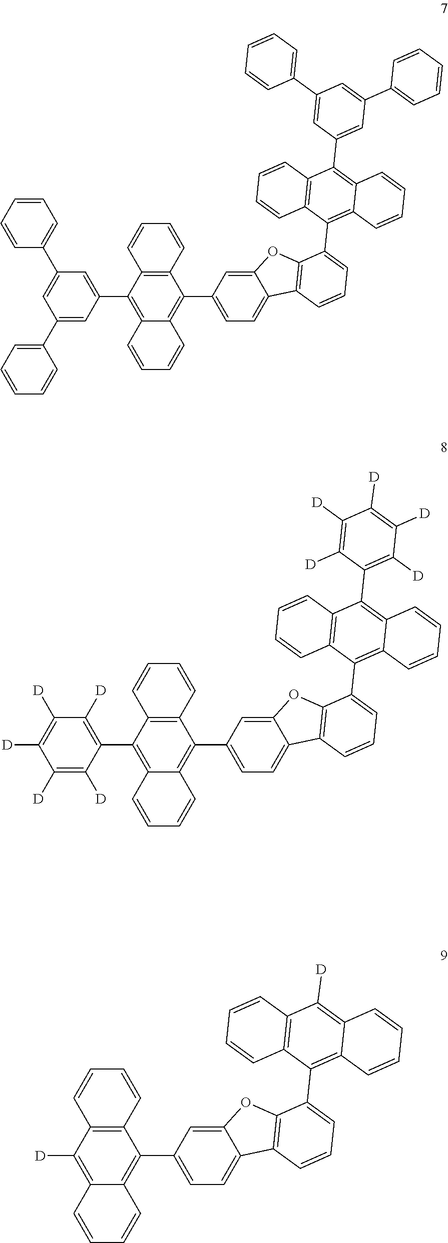 [SMILES: c1ccc(-c2cc(-c3ccccc3)cc(-c3c4ccccc4c(-c4ccc5c(c4)oc4c(-c6c7ccccc7c(-c7cc(-c8ccccc8)cc(-c8ccccc8)c7)c7ccccc67)cccc45)c4ccccc34)c2)cc1 ; [2H]c1c([2H])c([2H])c(-c2c3ccccc3c(-c3ccc4c(c3)oc3c(-c5c6ccccc6c(-c6c([2H])c([2H])c([2H])c([2H])c6[2H])c6ccccc56)cccc34)c3ccccc23)c([2H])c1[2H] ; [2H]c1c2ccccc2c(-c2ccc3c(c2)oc2c(-c4c5ccccc5c([2H])c5ccccc45)cccc23)c2ccccc12]

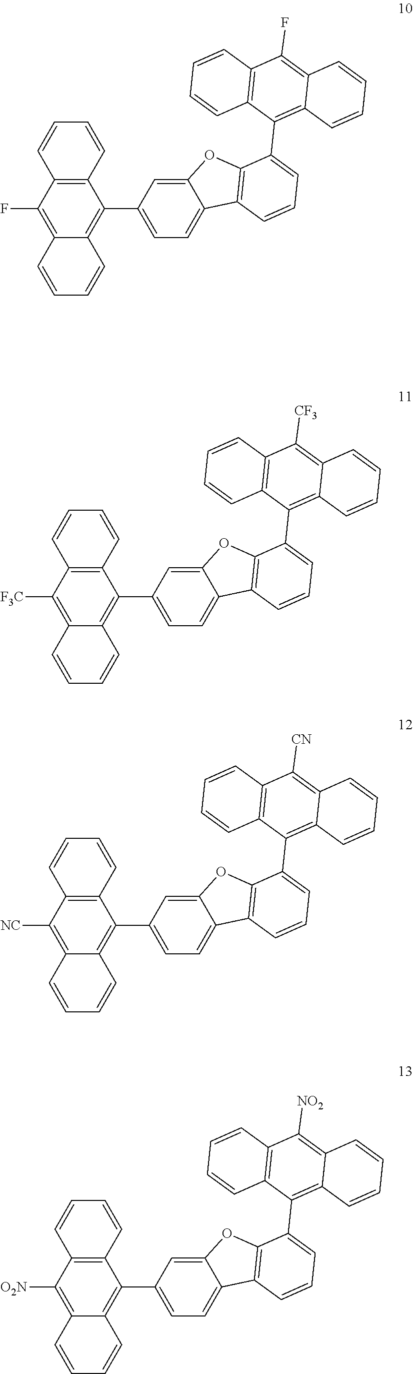 [SMILES: Fc1c2ccccc2c(-c2ccc3c(c2)oc2c(-c4c5ccccc5c(F)c5ccccc45)cccc23)c2ccccc12 ; FC(F)(F)c1c2ccccc2c(-c2ccc3c(c2)oc2c(-c4c5ccccc5c(C(F)(F)F)c5ccccc45)cccc23)c2ccccc12 ; N#Cc1c2ccccc2c(-c2ccc3c(c2)oc2c(-c4c5ccccc5c(C#N)c5ccccc45)cccc23)c2ccccc12 ; O=[N+]([O-])c1c2ccccc2c(-c2ccc3c(c2)oc2c(-c4c5ccccc5c([N+](=O)[O-])c5ccccc45)cccc23)c2ccccc12]

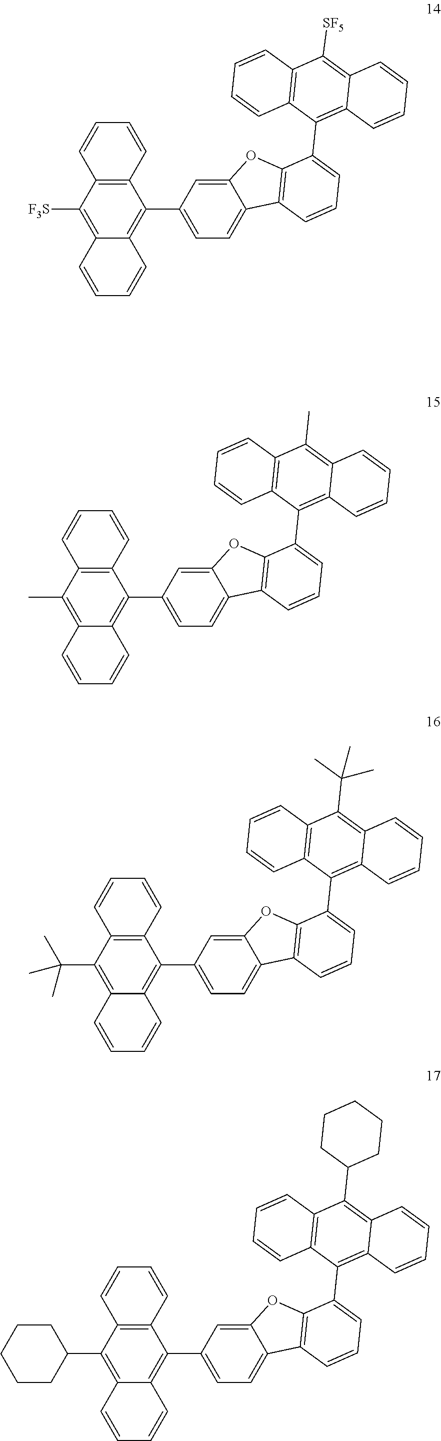

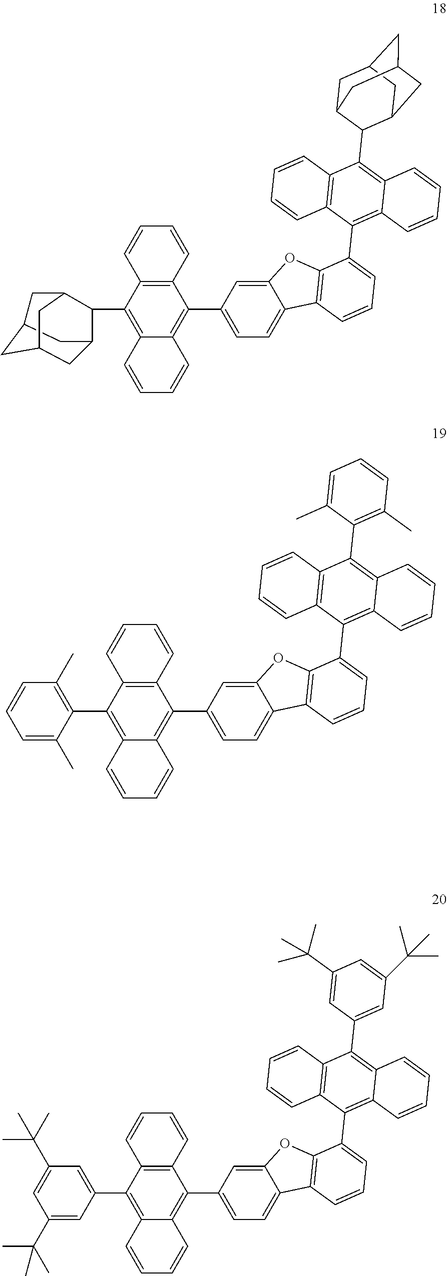 [SMILES: c1ccc2c(C3C4CC5CC(C4)CC3C5)c3ccccc3c(-c3ccc4c(c3)oc3c(-c5c6ccccc6c(C6C7CC8CC(C7)CC6C8)c6ccccc56)cccc34)c2c1 ; Cc1cccc(C)c1-c1c2ccccc2c(-c2ccc3c(c2)oc2c(-c4c5ccccc5c(-c5c(C)cccc5C)c5ccccc45)cccc23)c2ccccc12 ; CC(C)(C)c1cc(-c2c3ccccc3c(-c3ccc4c(c3)oc3c(-c5c6ccccc6c(-c6cc(C(C)(C)C)cc(C(C)(C)C)c6)c6ccccc56)cccc34)c3ccccc23)cc(C(C)(C)C)c1]

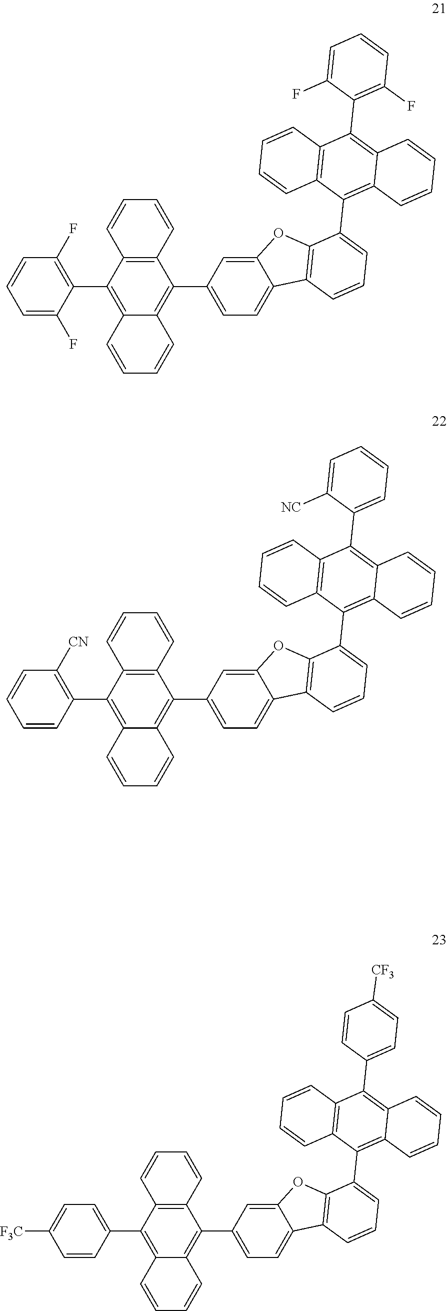 [SMILES: Fc1cccc(F)c1-c1c2ccccc2c(-c2ccc3c(c2)oc2c(-c4c5ccccc5c(-c5c(F)cccc5F)c5ccccc45)cccc23)c2ccccc12 ; N#Cc1ccccc1-c1c2ccccc2c(-c2ccc3c(c2)oc2c(-c4c5ccccc5c(-c5ccccc5C#N)c5ccccc45)cccc23)c2ccccc12 ; FC(F)(F)c1ccc(-c2c3ccccc3c(-c3ccc4c(c3)oc3c(-c5c6ccccc6c(-c6ccc(C(F)(F)F)cc6)c6ccccc56)cccc34)c3ccccc23)cc1]

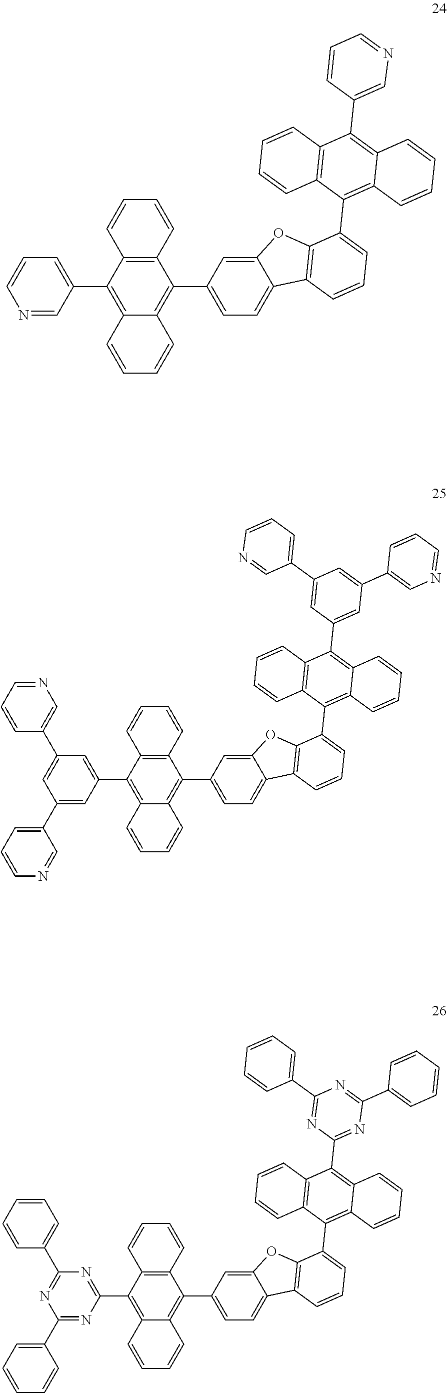 [SMILES: c1cncc(-c2c3ccccc3c(-c3ccc4c(c3)oc3c(-c5c6ccccc6c(-c6cccnc6)c6ccccc56)cccc34)c3ccccc23)c1 ; c1cncc(-c2cc(-c3cccnc3)cc(-c3c4ccccc4c(-c4ccc5c(c4)oc4c(-c6c7ccccc7c(-c7cc(-c8cccnc8)cc(-c8cccnc8)c7)c7ccccc67)cccc45)c4ccccc34)c2)c1 ; c1ccc(-c2nc(-c3ccccc3)nc(-c3c4ccccc4c(-c4ccc5c(c4)oc4c(-c6c7ccccc7c(-c7nc(-c8ccccc8)nc(-c8ccccc8)n7)c7ccccc67)cccc45)c4ccccc34)n2)cc1]

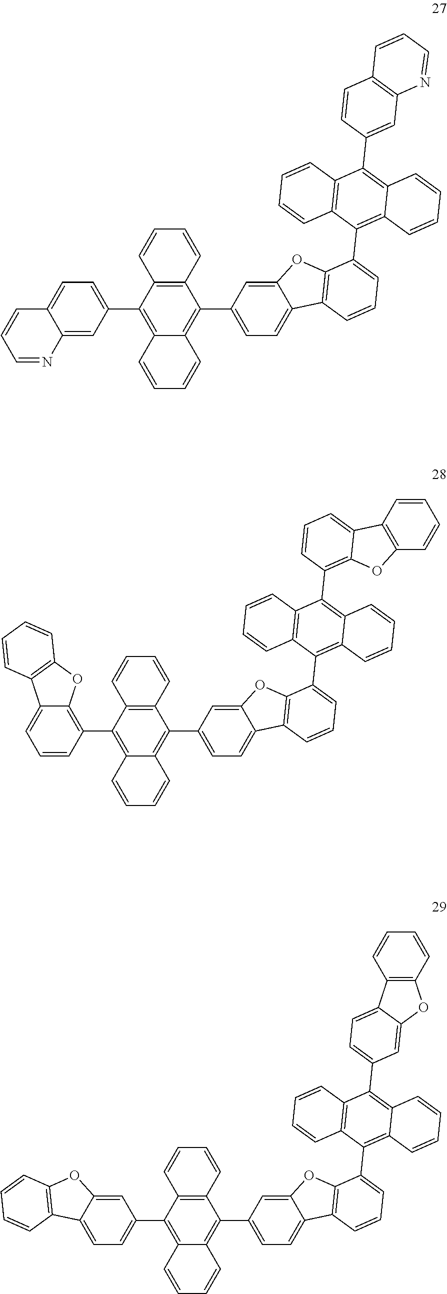 [SMILES: c1cnc2cc(-c3c4ccccc4c(-c4ccc5c(c4)oc4c(-c6c7ccccc7c(-c7ccc8cccnc8c7)c7ccccc67)cccc45)c4ccccc34)ccc2c1 ; c1ccc2c(c1)oc1c(-c3c4ccccc4c(-c4ccc5c(c4)oc4c(-c6c7ccccc7c(-c7cccc8c7oc7ccccc78)c7ccccc67)cccc45)c4ccccc34)cccc12 ; c1ccc2c(c1)oc1cc(-c3c4ccccc4c(-c4ccc5c(c4)oc4c(-c6c7ccccc7c(-c7ccc8c(c7)oc7ccccc78)c7ccccc67)cccc45)c4ccccc34)ccc12]

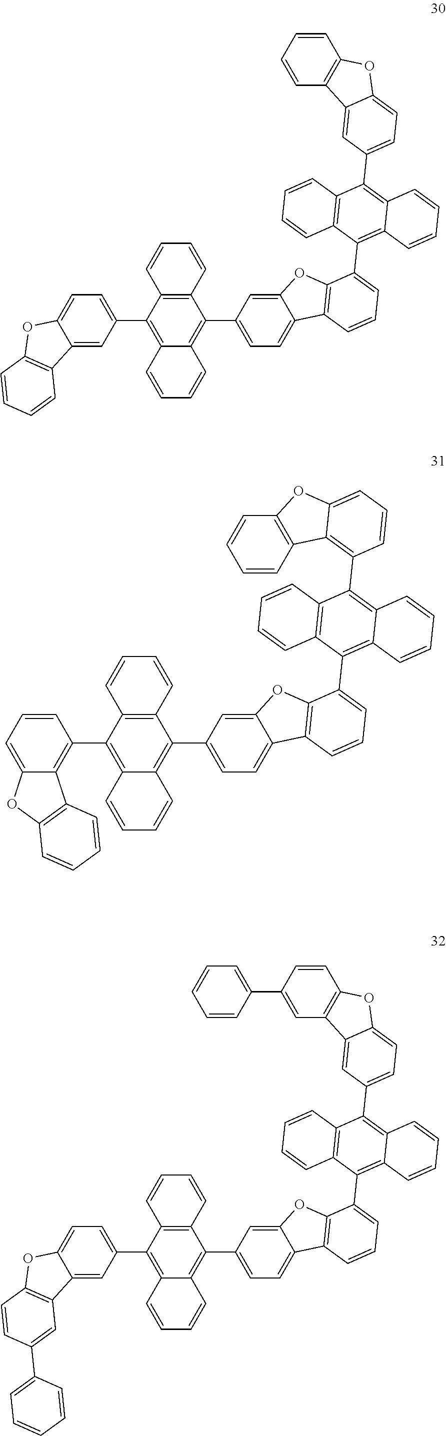 [SMILES: c1ccc2c(c1)oc1ccc(-c3c4ccccc4c(-c4ccc5c(c4)oc4c(-c6c7ccccc7c(-c7ccc8oc9ccccc9c8c7)c7ccccc67)cccc45)c4ccccc34)cc12 ; c1ccc2c(c1)oc1cccc(-c3c4ccccc4c(-c4ccc5c(c4)oc4c(-c6c7ccccc7c(-c7cccc8oc9ccccc9c78)c7ccccc67)cccc45)c4ccccc34)c12 ; c1ccc(-c2ccc3oc4ccc(-c5c6ccccc6c(-c6ccc7c(c6)oc6c(-c8c9ccccc9c(-c9ccc%10oc%11ccc(-c%12ccccc%12)cc%11c%10c9)c9ccccc89)cccc67)c6ccccc56)cc4c3c2)cc1]

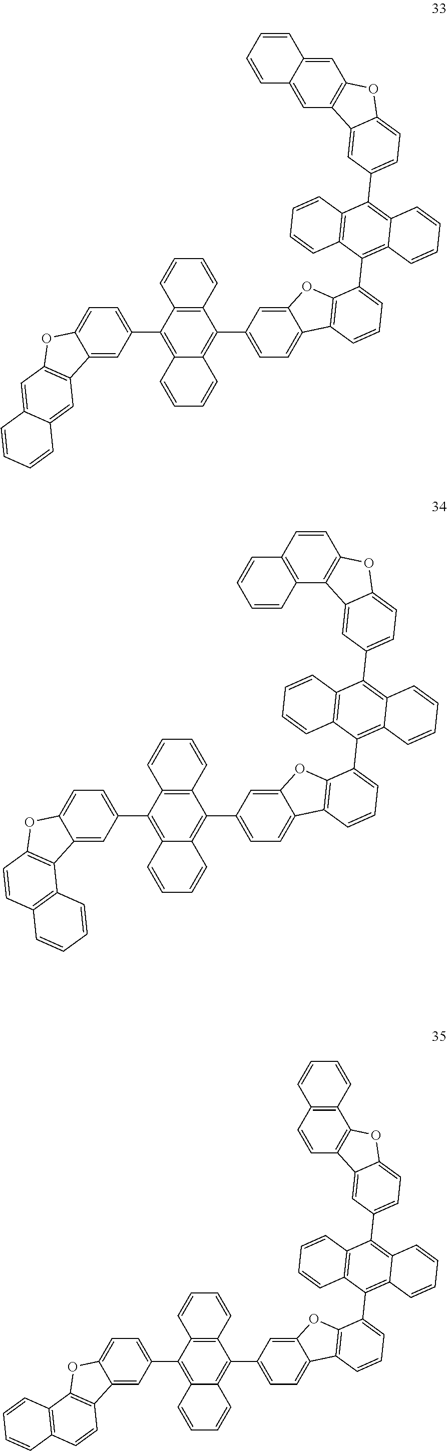 [SMILES: c1ccc2cc3c(cc2c1)oc1ccc(-c2c4ccccc4c(-c4ccc5c(c4)oc4c(-c6c7ccccc7c(-c7ccc8oc9cc%10ccccc%10cc9c8c7)c7ccccc67)cccc45)c4ccccc24)cc13 ; c1ccc2c(c1)ccc1oc3ccc(-c4c5ccccc5c(-c5ccc6c(c5)oc5c(-c7c8ccccc8c(-c8ccc9oc%10ccc%11ccccc%11c%10c9c8)c8ccccc78)cccc56)c5ccccc45)cc3c12 ; c1ccc2c(c1)ccc1c3cc(-c4c5ccccc5c(-c5ccc6c(c5)oc5c(-c7c8ccccc8c(-c8ccc9oc%10c%11ccccc%11ccc%10c9c8)c8ccccc78)cccc56)c5ccccc45)ccc3oc21]

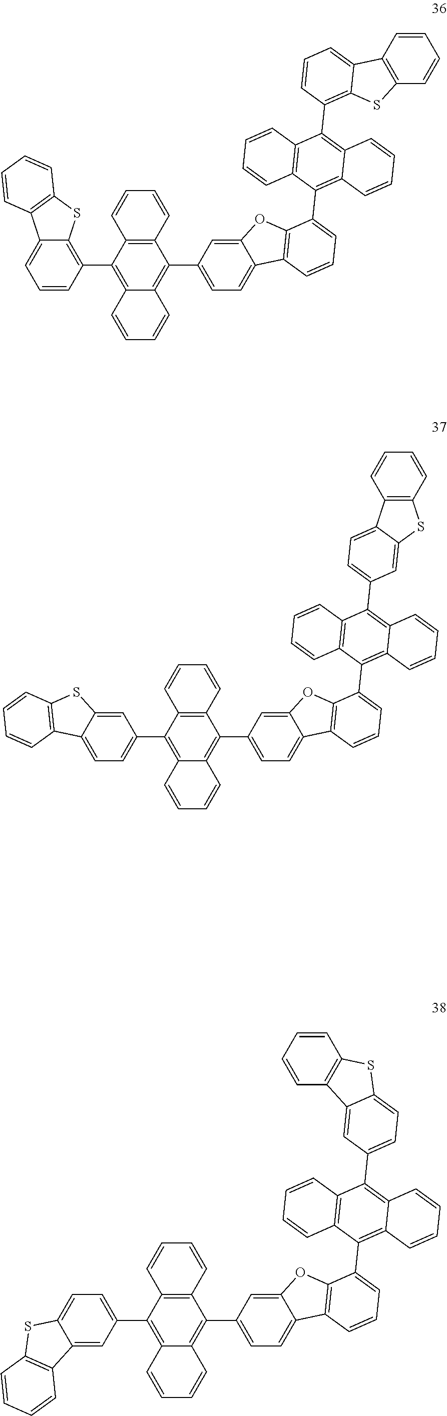 [SMILES: c1ccc2c(c1)sc1c(-c3c4ccccc4c(-c4ccc5c(c4)oc4c(-c6c7ccccc7c(-c7cccc8c7sc7ccccc78)c7ccccc67)cccc45)c4ccccc34)cccc12 ; c1ccc2c(c1)sc1cc(-c3c4ccccc4c(-c4ccc5c(c4)oc4c(-c6c7ccccc7c(-c7ccc8c(c7)sc7ccccc78)c7ccccc67)cccc45)c4ccccc34)ccc12 ; c1ccc2c(c1)sc1ccc(-c3c4ccccc4c(-c4ccc5c(c4)oc4c(-c6c7ccccc7c(-c7ccc8sc9ccccc9c8c7)c7ccccc67)cccc45)c4ccccc34)cc12]

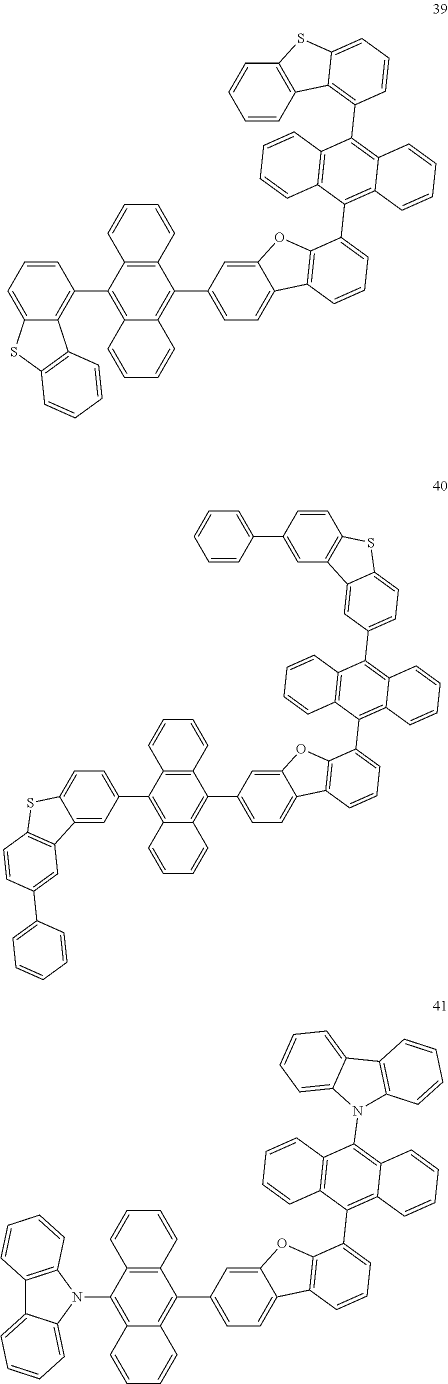 [SMILES: c1ccc2c(c1)sc1cccc(-c3c4ccccc4c(-c4ccc5c(c4)oc4c(-c6c7ccccc7c(-c7cccc8sc9ccccc9c78)c7ccccc67)cccc45)c4ccccc34)c12 ; c1ccc(-c2ccc3sc4ccc(-c5c6ccccc6c(-c6ccc7c(c6)oc6c(-c8c9ccccc9c(-c9ccc%10sc%11ccc(-c%12ccccc%12)cc%11c%10c9)c9ccccc89)cccc67)c6ccccc56)cc4c3c2)cc1 ; c1ccc2c(-n3c4ccccc4c4ccccc43)c3ccccc3c(-c3ccc4c(c3)oc3c(-c5c6ccccc6c(-n6c7ccccc7c7ccccc76)c6ccccc56)cccc34)c2c1]

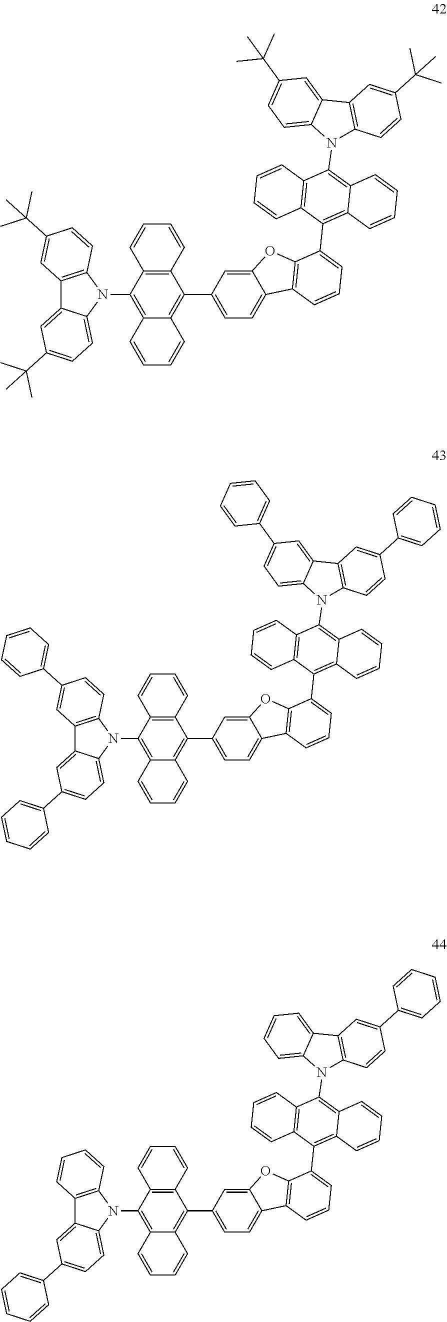 [SMILES: CC(C)(C)c1ccc2c(c1)c1cc(C(C)(C)C)ccc1n2-c1c2ccccc2c(-c2ccc3c(c2)oc2c(-c4c5ccccc5c(-n5c6ccc(C(C)(C)C)cc6c6cc(C(C)(C)C)ccc65)c5ccccc45)cccc23)c2ccccc12 ; c1ccc(-c2ccc3c(c2)c2cc(-c4ccccc4)ccc2n3-c2c3ccccc3c(-c3ccc4c(c3)oc3c(-c5c6ccccc6c(-n6c7ccc(-c8ccccc8)cc7c7cc(-c8ccccc8)ccc76)c6ccccc56)cccc34)c3ccccc23)cc1 ; c1ccc(-c2ccc3c(c2)c2ccccc2n3-c2c3ccccc3c(-c3ccc4c(c3)oc3c(-c5c6ccccc6c(-n6c7ccccc7c7cc(-c8ccccc8)ccc76)c6ccccc56)cccc34)c3ccccc23)cc1]

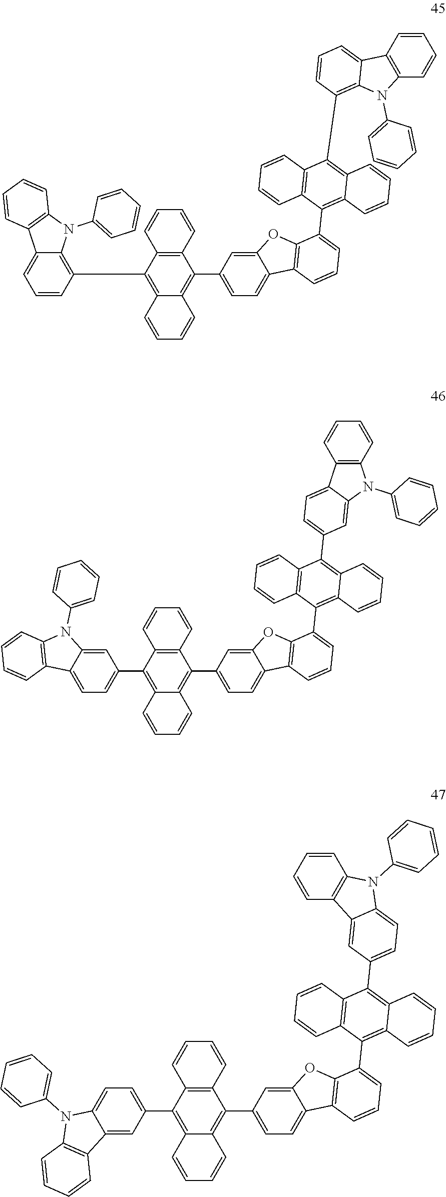 [SMILES: c1ccc(-n2c3ccccc3c3cccc(-c4c5ccccc5c(-c5ccc6c(c5)oc5c(-c7c8ccccc8c(-c8cccc9c%10ccccc%10n(-c%10ccccc%10)c89)c8ccccc78)cccc56)c5ccccc45)c32)cc1 ; c1ccc(-n2c3ccccc3c3ccc(-c4c5ccccc5c(-c5ccc6c(c5)oc5c(-c7c8ccccc8c(-c8ccc9c%10ccccc%10n(-c%10ccccc%10)c9c8)c8ccccc78)cccc56)c5ccccc45)cc32)cc1 ; c1ccc(-n2c3ccccc3c3cc(-c4c5ccccc5c(-c5ccc6c(c5)oc5c(-c7c8ccccc8c(-c8ccc9c(c8)c8ccccc8n9-c8ccccc8)c8ccccc78)cccc56)c5ccccc45)ccc32)cc1]

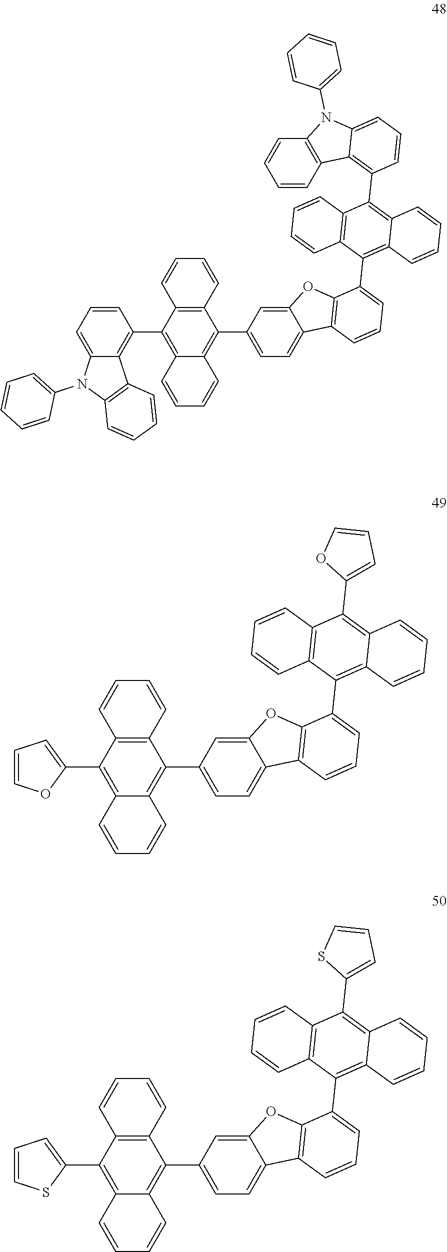 [SMILES: c1ccc(-n2c3ccccc3c3c(-c4c5ccccc5c(-c5ccc6c(c5)oc5c(-c7c8ccccc8c(-c8cccc9c8c8ccccc8n9-c8ccccc8)c8ccccc78)cccc56)c5ccccc45)cccc32)cc1 ; c1coc(-c2c3ccccc3c(-c3ccc4c(c3)oc3c(-c5c6ccccc6c(-c6ccco6)c6ccccc56)cccc34)c3ccccc23)c1 ; c1csc(-c2c3ccccc3c(-c3ccc4c(c3)oc3c(-c5c6ccccc6c(-c6cccs6)c6ccccc56)cccc34)c3ccccc23)c1]

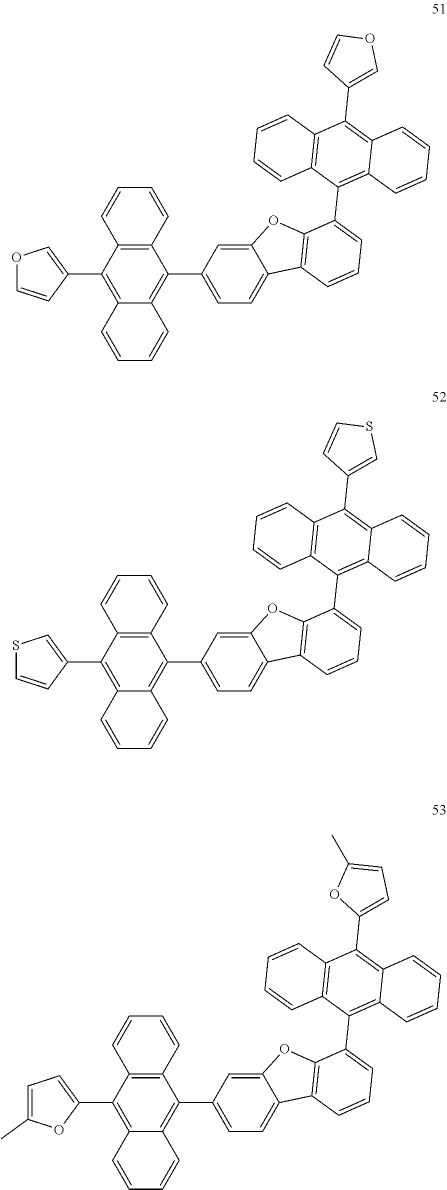 [SMILES: c1ccc2c(-c3ccc4c(c3)oc3c(-c5c6ccccc6c(-c6ccoc6)c6ccccc56)cccc34)c3ccccc3c(-c3ccoc3)c2c1 ; c1ccc2c(-c3ccc4c(c3)oc3c(-c5c6ccccc6c(-c6ccsc6)c6ccccc56)cccc34)c3ccccc3c(-c3ccsc3)c2c1 ; Cc1ccc(-c2c3ccccc3c(-c3ccc4c(c3)oc3c(-c5c6ccccc6c(-c6ccc(C)o6)c6ccccc56)cccc34)c3ccccc23)o1]

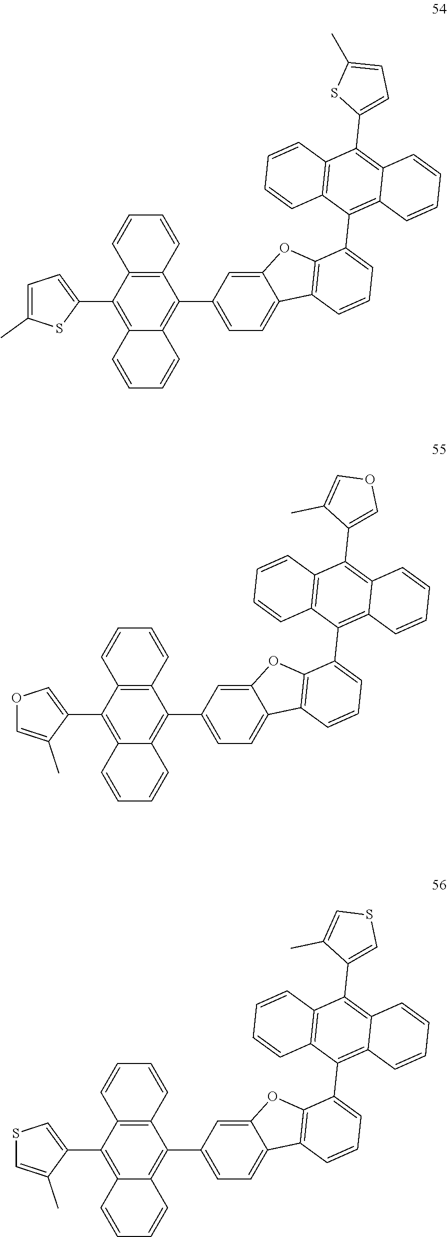 [SMILES: Cc1ccc(-c2c3ccccc3c(-c3ccc4c(c3)oc3c(-c5c6ccccc6c(-c6ccc(C)s6)c6ccccc56)cccc34)c3ccccc23)s1 ; Cc1cocc1-c1c2ccccc2c(-c2ccc3c(c2)oc2c(-c4c5ccccc5c(-c5cocc5C)c5ccccc45)cccc23)c2ccccc12 ; Cc1cscc1-c1c2ccccc2c(-c2ccc3c(c2)oc2c(-c4c5ccccc5c(-c5cscc5C)c5ccccc45)cccc23)c2ccccc12]

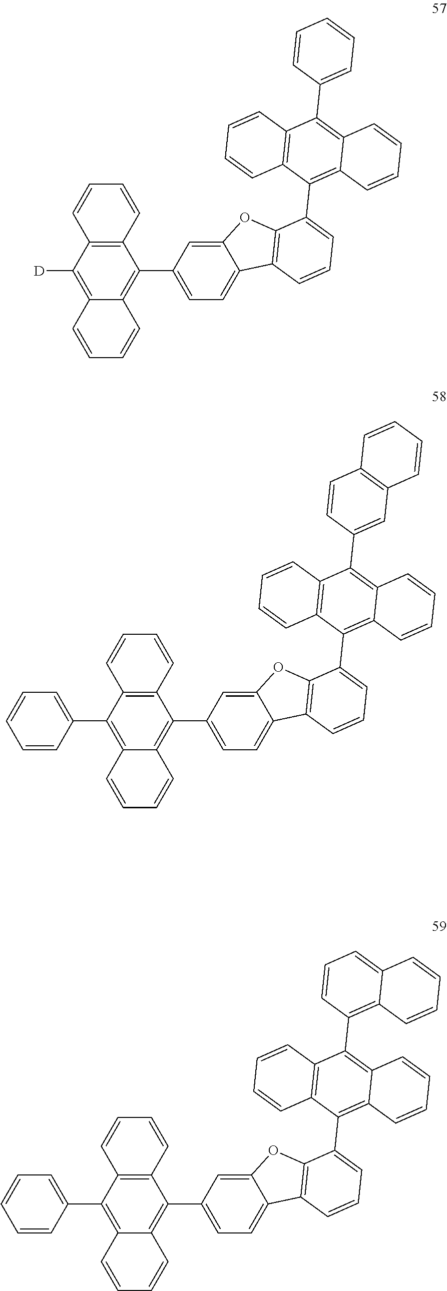 [SMILES: [2H]c1c2ccccc2c(-c2ccc3c(c2)oc2c(-c4c5ccccc5c(-c5ccccc5)c5ccccc45)cccc23)c2ccccc12 ; c1ccc(-c2c3ccccc3c(-c3ccc4c(c3)oc3c(-c5c6ccccc6c(-c6ccc7ccccc7c6)c6ccccc56)cccc34)c3ccccc23)cc1 ; c1ccc(-c2c3ccccc3c(-c3ccc4c(c3)oc3c(-c5c6ccccc6c(-c6cccc7ccccc67)c6ccccc56)cccc34)c3ccccc23)cc1]

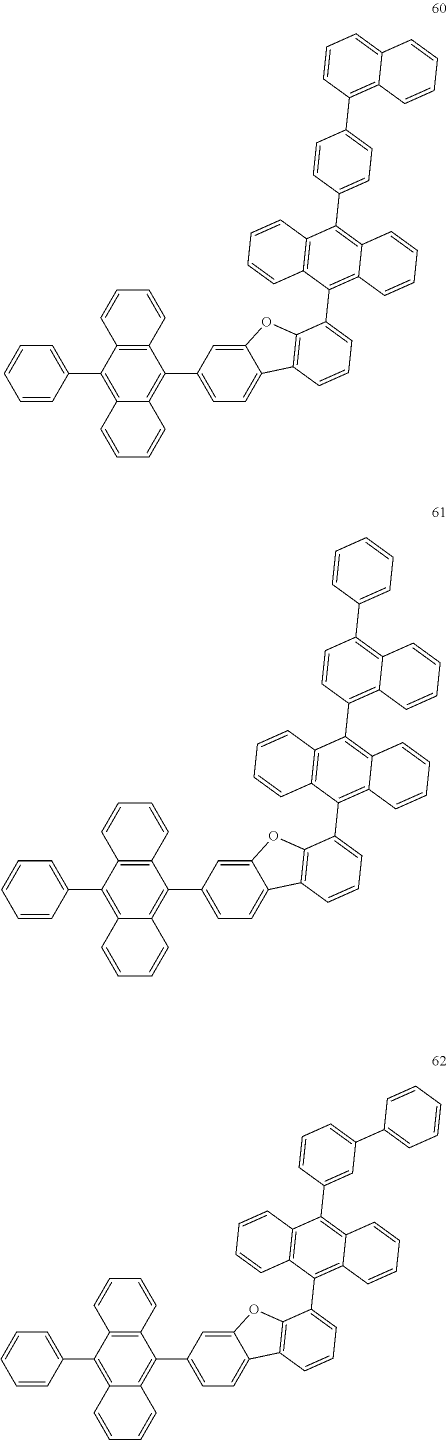 [SMILES: c1ccc(-c2c3ccccc3c(-c3ccc4c(c3)oc3c(-c5c6ccccc6c(-c6ccc(-c7cccc8ccccc78)cc6)c6ccccc56)cccc34)c3ccccc23)cc1 ; c1ccc(-c2ccc(-c3c4ccccc4c(-c4cccc5c4oc4cc(-c6c7ccccc7c(-c7ccccc7)c7ccccc67)ccc45)c4ccccc34)c3ccccc23)cc1 ; c1ccc(-c2cccc(-c3c4ccccc4c(-c4cccc5c4oc4cc(-c6c7ccccc7c(-c7ccccc7)c7ccccc67)ccc45)c4ccccc34)c2)cc1]

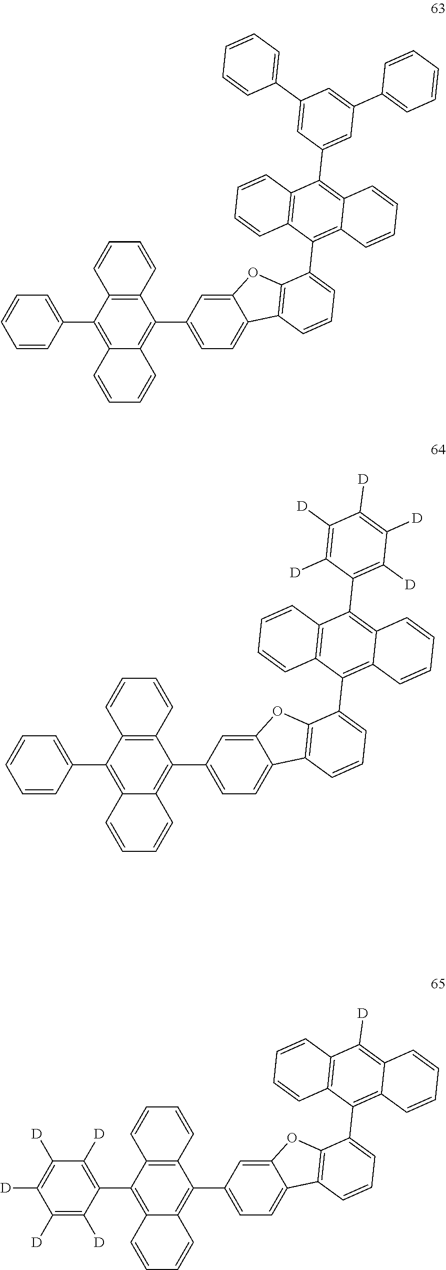 [SMILES: c1ccc(-c2cc(-c3ccccc3)cc(-c3c4ccccc4c(-c4cccc5c4oc4cc(-c6c7ccccc7c(-c7ccccc7)c7ccccc67)ccc45)c4ccccc34)c2)cc1 ; [2H]c1c([2H])c([2H])c(-c2c3ccccc3c(-c3cccc4c3oc3cc(-c5c6ccccc6c(-c6ccccc6)c6ccccc56)ccc34)c3ccccc23)c([2H])c1[2H] ; [2H]c1c([2H])c([2H])c(-c2c3ccccc3c(-c3ccc4c(c3)oc3c(-c5c6ccccc6c([2H])c6ccccc56)cccc34)c3ccccc23)c([2H])c1[2H]]

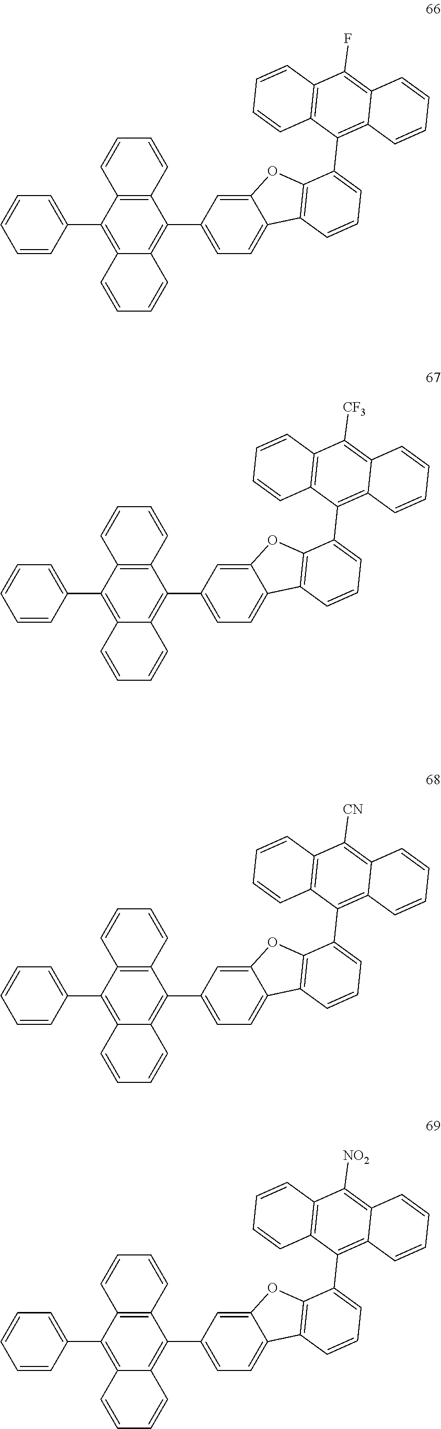 [SMILES: Fc1c2ccccc2c(-c2cccc3c2oc2cc(-c4c5ccccc5c(-c5ccccc5)c5ccccc45)ccc23)c2ccccc12 ; FC(F)(F)c1c2ccccc2c(-c2cccc3c2oc2cc(-c4c5ccccc5c(-c5ccccc5)c5ccccc45)ccc23)c2ccccc12 ; N#Cc1c2ccccc2c(-c2cccc3c2oc2cc(-c4c5ccccc5c(-c5ccccc5)c5ccccc45)ccc23)c2ccccc12 ; O=[N+]([O-])c1c2ccccc2c(-c2cccc3c2oc2cc(-c4c5ccccc5c(-c5ccccc5)c5ccccc45)ccc23)c2ccccc12]

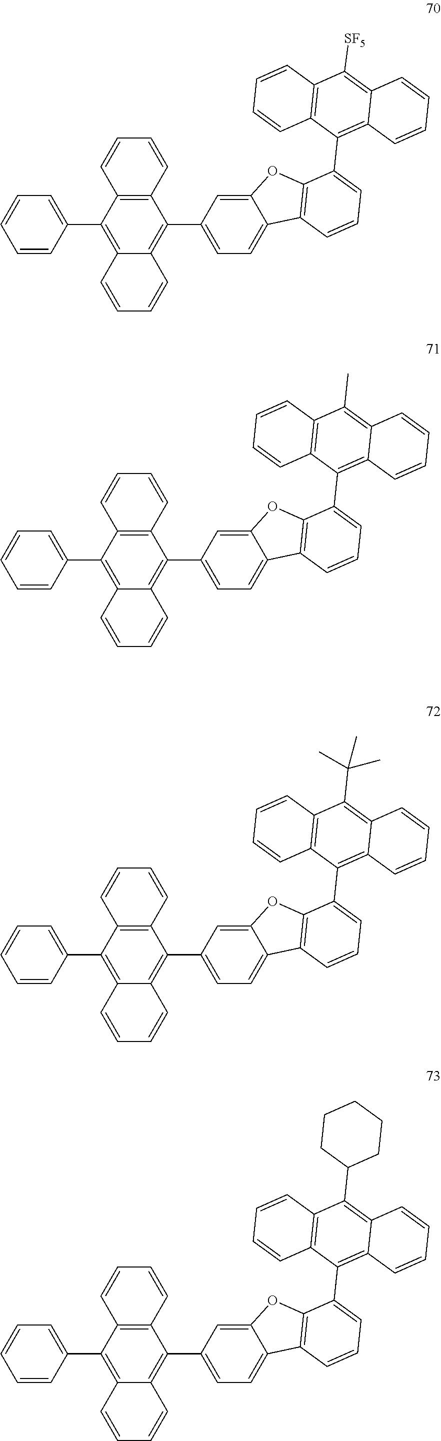

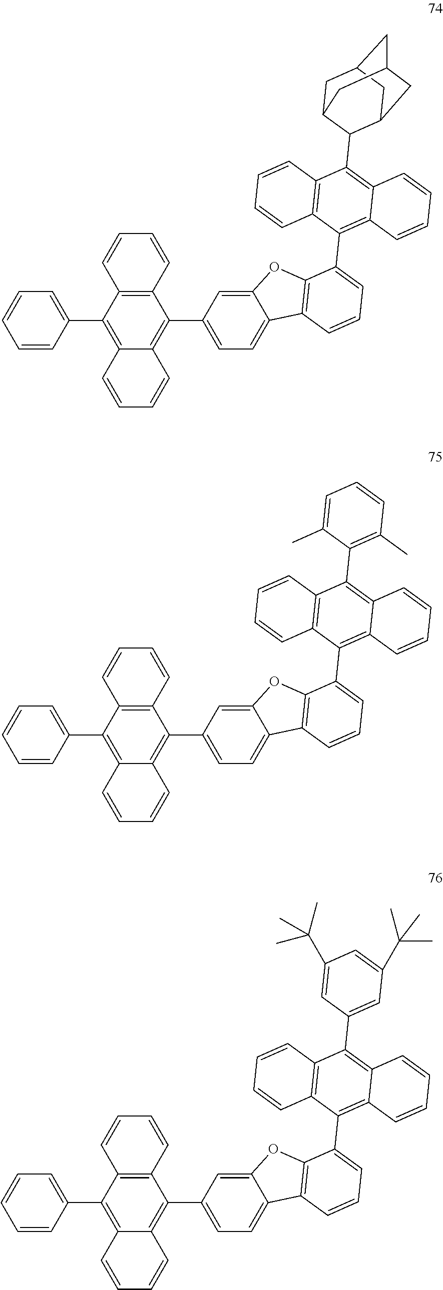 [SMILES: c1ccc(-c2c3ccccc3c(-c3ccc4c(c3)oc3c(-c5c6ccccc6c(C6C7CC8CC(C7)CC6C8)c6ccccc56)cccc34)c3ccccc23)cc1 ; Cc1cccc(C)c1-c1c2ccccc2c(-c2cccc3c2oc2cc(-c4c5ccccc5c(-c5ccccc5)c5ccccc45)ccc23)c2ccccc12 ; CC(C)(C)c1cc(-c2c3ccccc3c(-c3cccc4c3oc3cc(-c5c6ccccc6c(-c6ccccc6)c6ccccc56)ccc34)c3ccccc23)cc(C(C)(C)C)c1]

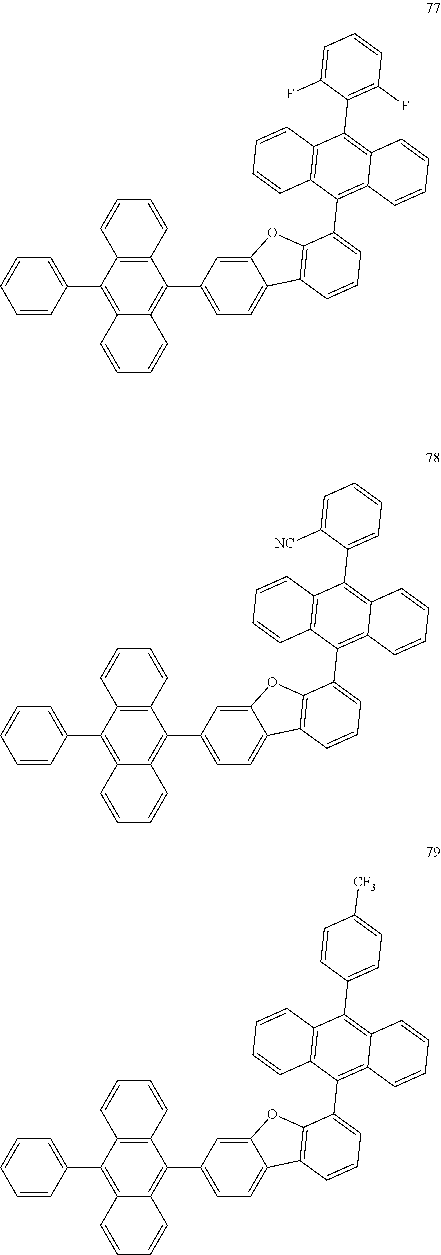 [SMILES: Fc1cccc(F)c1-c1c2ccccc2c(-c2cccc3c2oc2cc(-c4c5ccccc5c(-c5ccccc5)c5ccccc45)ccc23)c2ccccc12 ; N#Cc1ccccc1-c1c2ccccc2c(-c2cccc3c2oc2cc(-c4c5ccccc5c(-c5ccccc5)c5ccccc45)ccc23)c2ccccc12 ; FC(F)(F)c1ccc(-c2c3ccccc3c(-c3cccc4c3oc3cc(-c5c6ccccc6c(-c6ccccc6)c6ccccc56)ccc34)c3ccccc23)cc1]

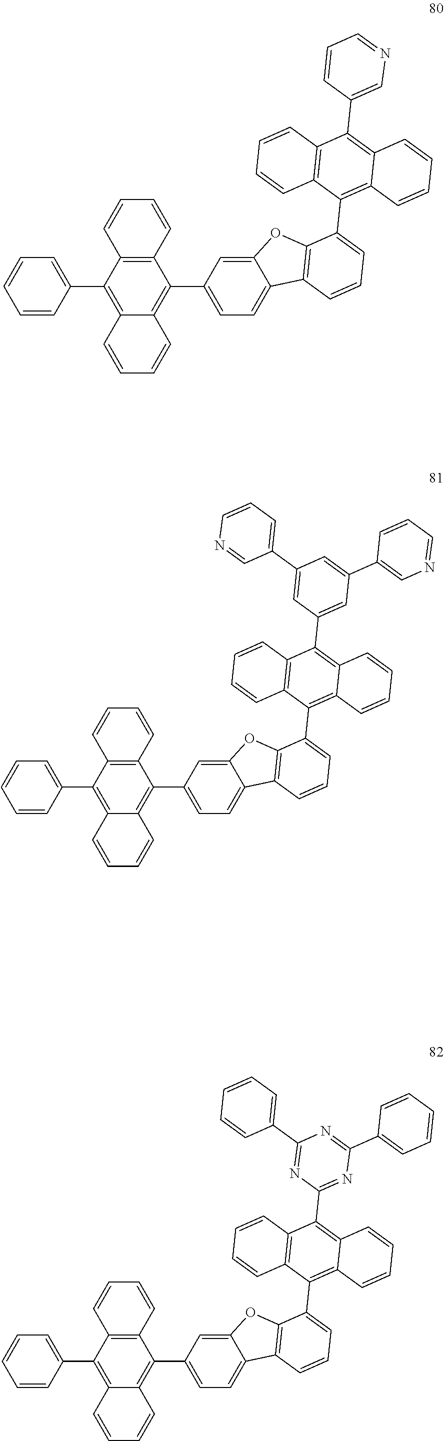 [SMILES: c1ccc(-c2c3ccccc3c(-c3ccc4c(c3)oc3c(-c5c6ccccc6c(-c6cccnc6)c6ccccc56)cccc34)c3ccccc23)cc1 ; c1ccc(-c2c3ccccc3c(-c3ccc4c(c3)oc3c(-c5c6ccccc6c(-c6cc(-c7cccnc7)cc(-c7cccnc7)c6)c6ccccc56)cccc34)c3ccccc23)cc1 ; c1ccc(-c2nc(-c3ccccc3)nc(-c3c4ccccc4c(-c4cccc5c4oc4cc(-c6c7ccccc7c(-c7ccccc7)c7ccccc67)ccc45)c4ccccc34)n2)cc1]

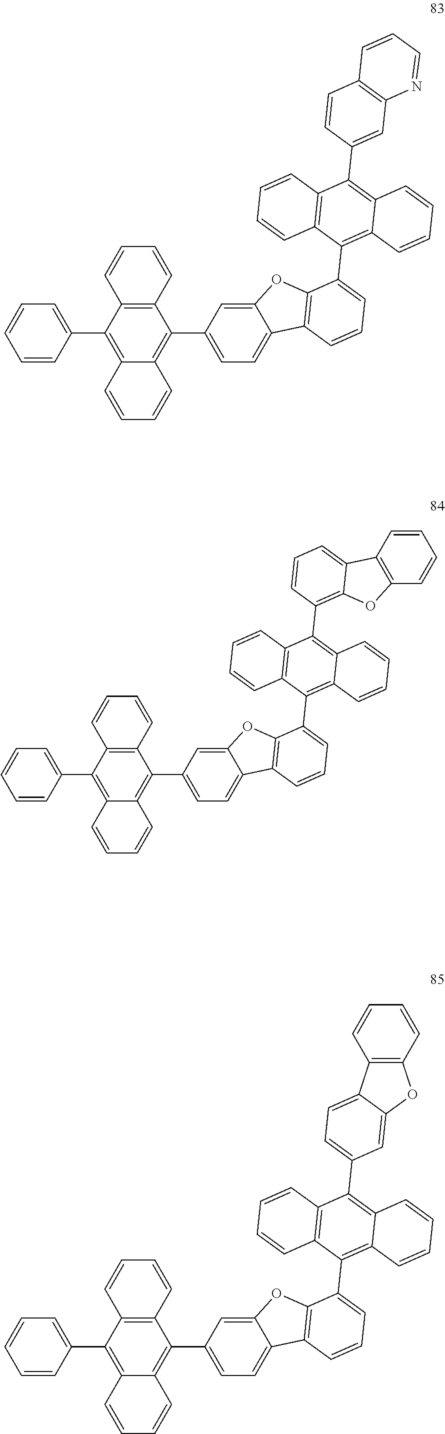 [SMILES: c1ccc(-c2c3ccccc3c(-c3ccc4c(c3)oc3c(-c5c6ccccc6c(-c6ccc7cccnc7c6)c6ccccc56)cccc34)c3ccccc23)cc1 ; c1ccc(-c2c3ccccc3c(-c3ccc4c(c3)oc3c(-c5c6ccccc6c(-c6cccc7c6oc6ccccc67)c6ccccc56)cccc34)c3ccccc23)cc1 ; c1ccc(-c2c3ccccc3c(-c3ccc4c(c3)oc3c(-c5c6ccccc6c(-c6ccc7c(c6)oc6ccccc67)c6ccccc56)cccc34)c3ccccc23)cc1]

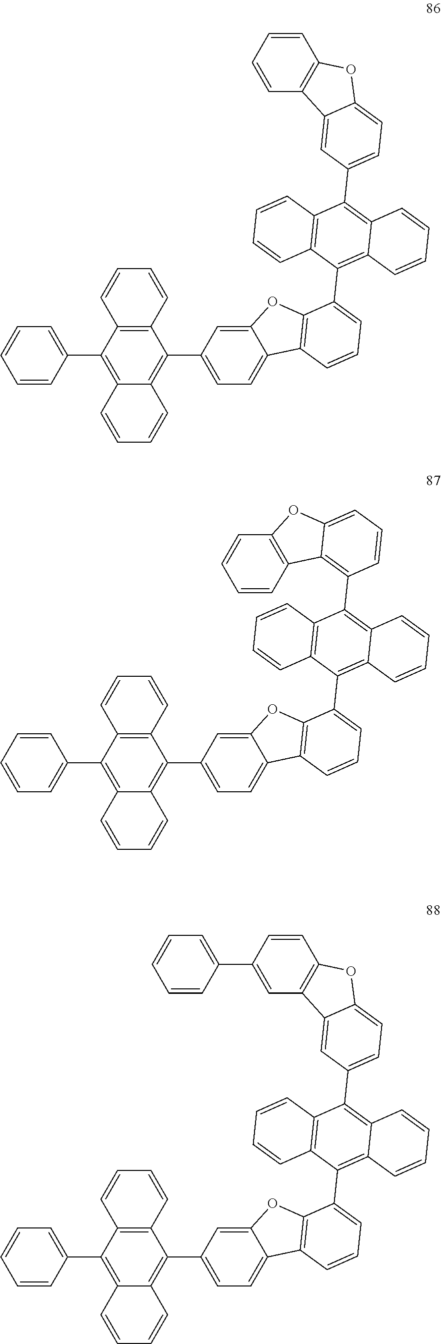 [SMILES: c1ccc(-c2c3ccccc3c(-c3ccc4c(c3)oc3c(-c5c6ccccc6c(-c6ccc7oc8ccccc8c7c6)c6ccccc56)cccc34)c3ccccc23)cc1 ; c1ccc(-c2c3ccccc3c(-c3ccc4c(c3)oc3c(-c5c6ccccc6c(-c6cccc7oc8ccccc8c67)c6ccccc56)cccc34)c3ccccc23)cc1 ; c1ccc(-c2ccc3oc4ccc(-c5c6ccccc6c(-c6cccc7c6oc6cc(-c8c9ccccc9c(-c9ccccc9)c9ccccc89)ccc67)c6ccccc56)cc4c3c2)cc1]

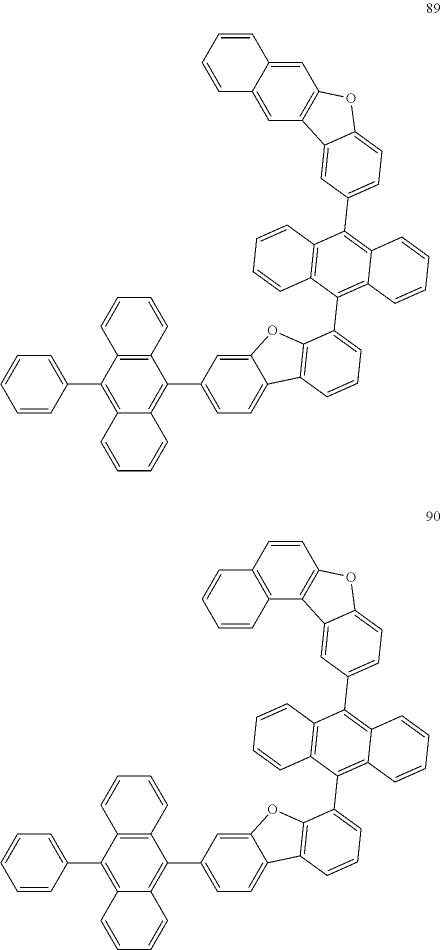 [SMILES: c1ccc(-c2c3ccccc3c(-c3ccc4c(c3)oc3c(-c5c6ccccc6c(-c6ccc7oc8cc9ccccc9cc8c7c6)c6ccccc56)cccc34)c3ccccc23)cc1 ; c1ccc(-c2c3ccccc3c(-c3ccc4c(c3)oc3c(-c5c6ccccc6c(-c6ccc7oc8ccc9ccccc9c8c7c6)c6ccccc56)cccc34)c3ccccc23)cc1]

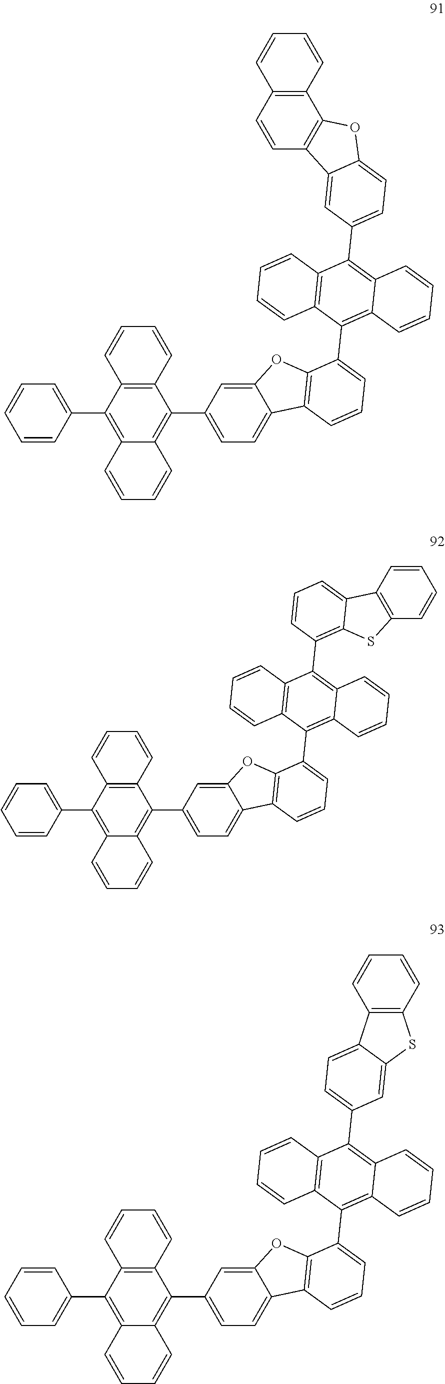 [SMILES: c1ccc(-c2c3ccccc3c(-c3ccc4c(c3)oc3c(-c5c6ccccc6c(-c6ccc7oc8c9ccccc9ccc8c7c6)c6ccccc56)cccc34)c3ccccc23)cc1 ; c1ccc(-c2c3ccccc3c(-c3ccc4c(c3)oc3c(-c5c6ccccc6c(-c6cccc7c6sc6ccccc67)c6ccccc56)cccc34)c3ccccc23)cc1 ; c1ccc(-c2c3ccccc3c(-c3ccc4c(c3)oc3c(-c5c6ccccc6c(-c6ccc7c(c6)sc6ccccc67)c6ccccc56)cccc34)c3ccccc23)cc1]

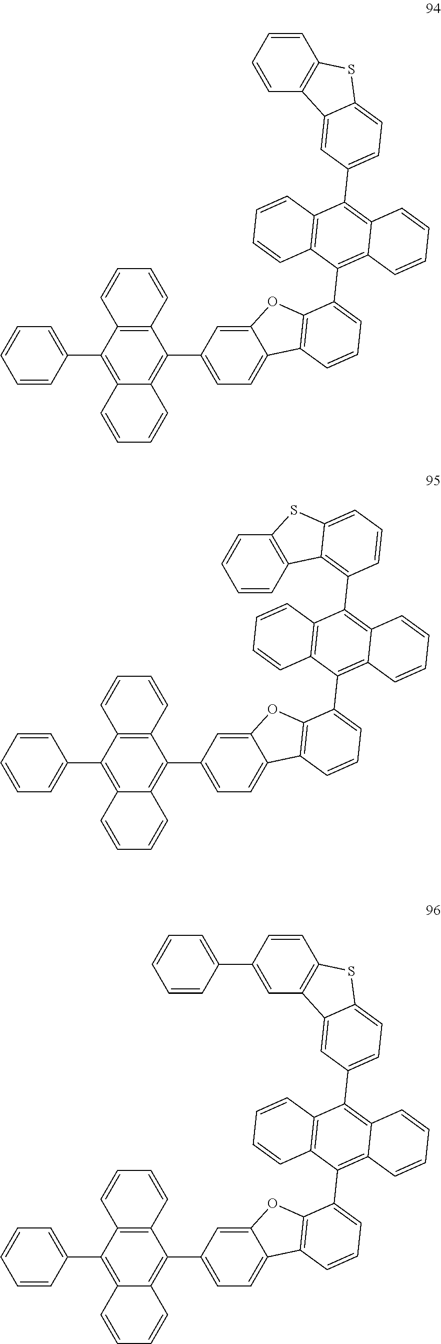 [SMILES: c1ccc(-c2c3ccccc3c(-c3ccc4c(c3)oc3c(-c5c6ccccc6c(-c6ccc7sc8ccccc8c7c6)c6ccccc56)cccc34)c3ccccc23)cc1 ; c1ccc(-c2c3ccccc3c(-c3ccc4c(c3)oc3c(-c5c6ccccc6c(-c6cccc7sc8ccccc8c67)c6ccccc56)cccc34)c3ccccc23)cc1 ; c1ccc(-c2ccc3sc4ccc(-c5c6ccccc6c(-c6cccc7c6oc6cc(-c8c9ccccc9c(-c9ccccc9)c9ccccc89)ccc67)c6ccccc56)cc4c3c2)cc1]

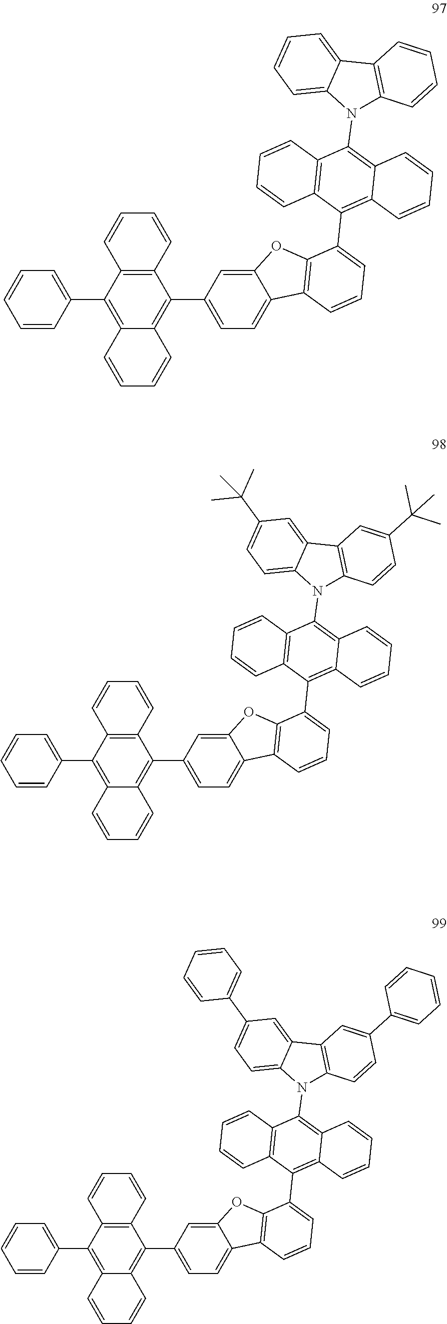 [SMILES: c1ccc(-c2c3ccccc3c(-c3ccc4c(c3)oc3c(-c5c6ccccc6c(-n6c7ccccc7c7ccccc76)c6ccccc56)cccc34)c3ccccc23)cc1 ; CC(C)(C)c1ccc2c(c1)c1cc(C(C)(C)C)ccc1n2-c1c2ccccc2c(-c2cccc3c2oc2cc(-c4c5ccccc5c(-c5ccccc5)c5ccccc45)ccc23)c2ccccc12 ; c1ccc(-c2ccc3c(c2)c2cc(-c4ccccc4)ccc2n3-c2c3ccccc3c(-c3cccc4c3oc3cc(-c5c6ccccc6c(-c6ccccc6)c6ccccc56)ccc34)c3ccccc23)cc1]

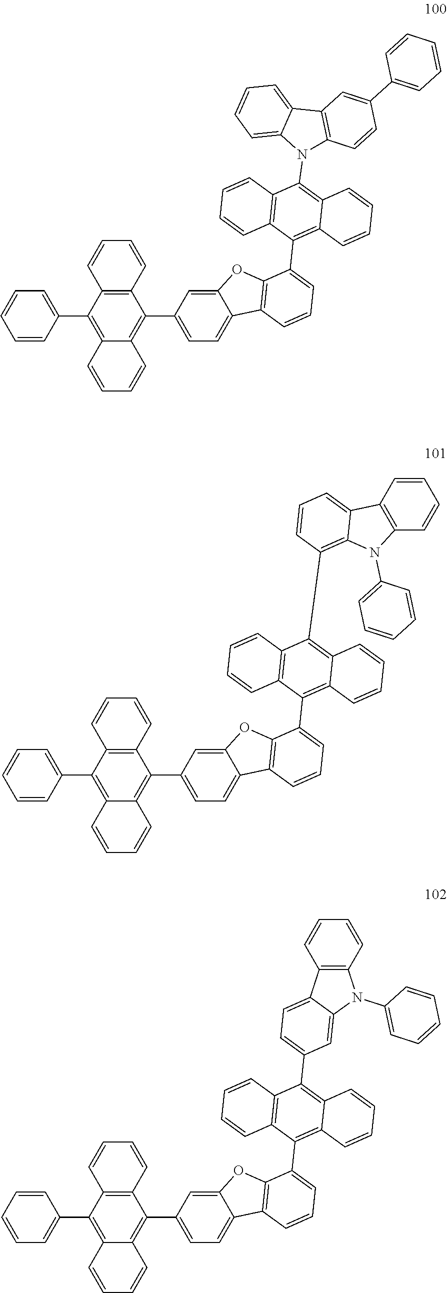 [SMILES: c1ccc(-c2ccc3c(c2)c2ccccc2n3-c2c3ccccc3c(-c3cccc4c3oc3cc(-c5c6ccccc6c(-c6ccccc6)c6ccccc56)ccc34)c3ccccc23)cc1 ; c1ccc(-c2c3ccccc3c(-c3ccc4c(c3)oc3c(-c5c6ccccc6c(-c6cccc7c8ccccc8n(-c8ccccc8)c67)c6ccccc56)cccc34)c3ccccc23)cc1 ; c1ccc(-c2c3ccccc3c(-c3ccc4c(c3)oc3c(-c5c6ccccc6c(-c6ccc7c8ccccc8n(-c8ccccc8)c7c6)c6ccccc56)cccc34)c3ccccc23)cc1]

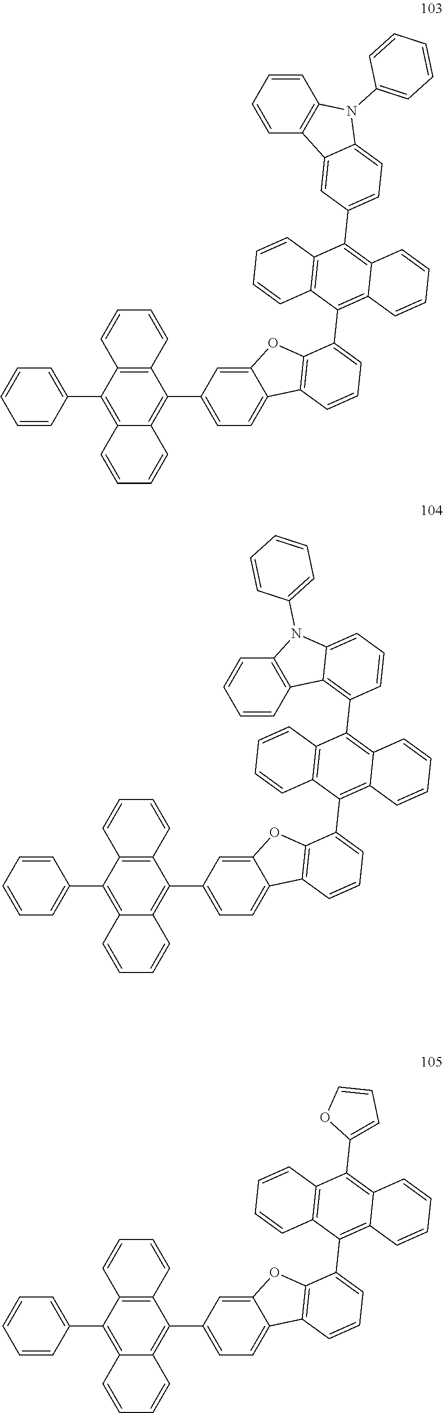 [SMILES: c1ccc(-c2c3ccccc3c(-c3ccc4c(c3)oc3c(-c5c6ccccc6c(-c6ccc7c(c6)c6ccccc6n7-c6ccccc6)c6ccccc56)cccc34)c3ccccc23)cc1 ; c1ccc(-c2c3ccccc3c(-c3ccc4c(c3)oc3c(-c5c6ccccc6c(-c6cccc7c6c6ccccc6n7-c6ccccc6)c6ccccc56)cccc34)c3ccccc23)cc1 ; c1ccc(-c2c3ccccc3c(-c3ccc4c(c3)oc3c(-c5c6ccccc6c(-c6ccco6)c6ccccc56)cccc34)c3ccccc23)cc1]

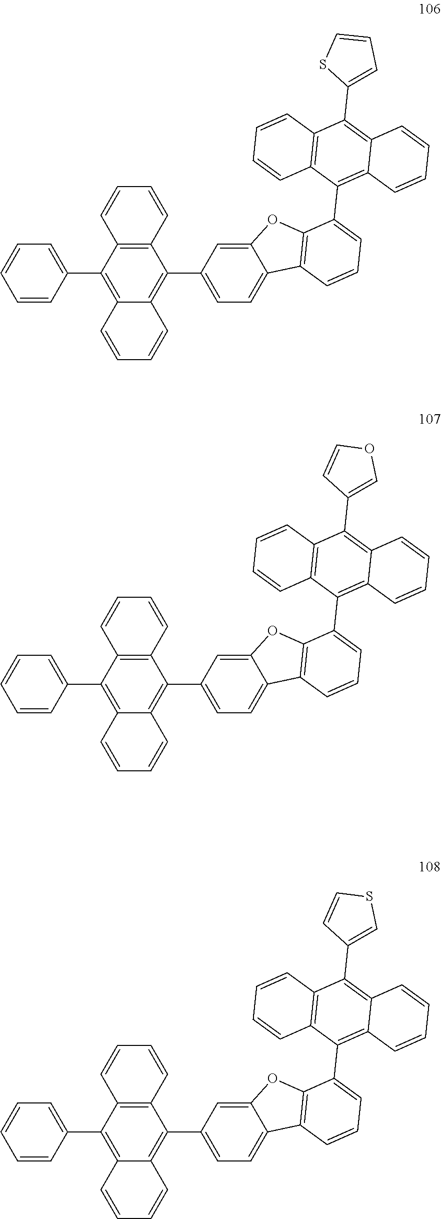 [SMILES: c1ccc(-c2c3ccccc3c(-c3ccc4c(c3)oc3c(-c5c6ccccc6c(-c6cccs6)c6ccccc56)cccc34)c3ccccc23)cc1 ; c1ccc(-c2c3ccccc3c(-c3ccc4c(c3)oc3c(-c5c6ccccc6c(-c6ccoc6)c6ccccc56)cccc34)c3ccccc23)cc1 ; c1ccc(-c2c3ccccc3c(-c3ccc4c(c3)oc3c(-c5c6ccccc6c(-c6ccsc6)c6ccccc56)cccc34)c3ccccc23)cc1]

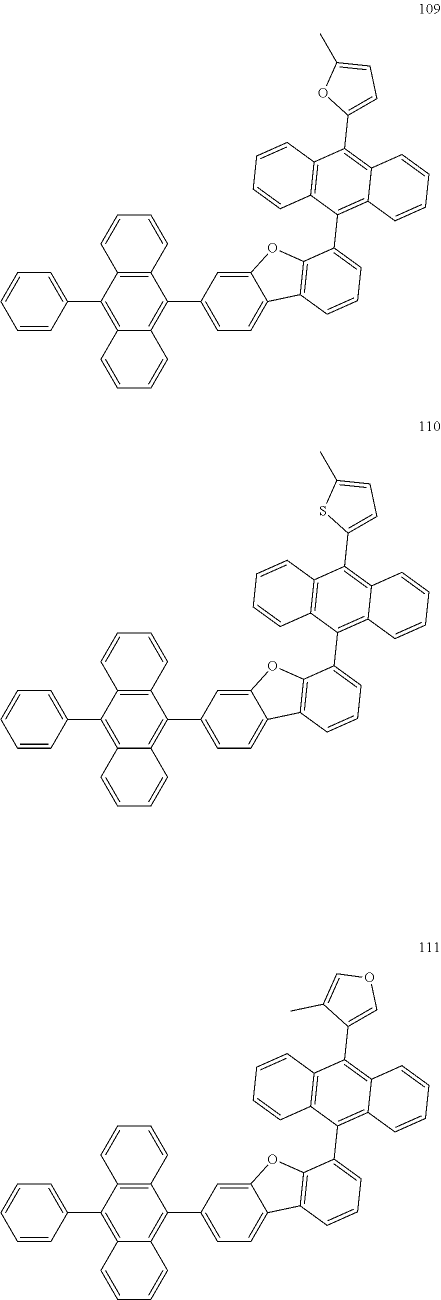 [SMILES: Cc1ccc(-c2c3ccccc3c(-c3cccc4c3oc3cc(-c5c6ccccc6c(-c6ccccc6)c6ccccc56)ccc34)c3ccccc23)o1 ; Cc1ccc(-c2c3ccccc3c(-c3cccc4c3oc3cc(-c5c6ccccc6c(-c6ccccc6)c6ccccc56)ccc34)c3ccccc23)s1 ; Cc1cocc1-c1c2ccccc2c(-c2cccc3c2oc2cc(-c4c5ccccc5c(-c5ccccc5)c5ccccc45)ccc23)c2ccccc12]

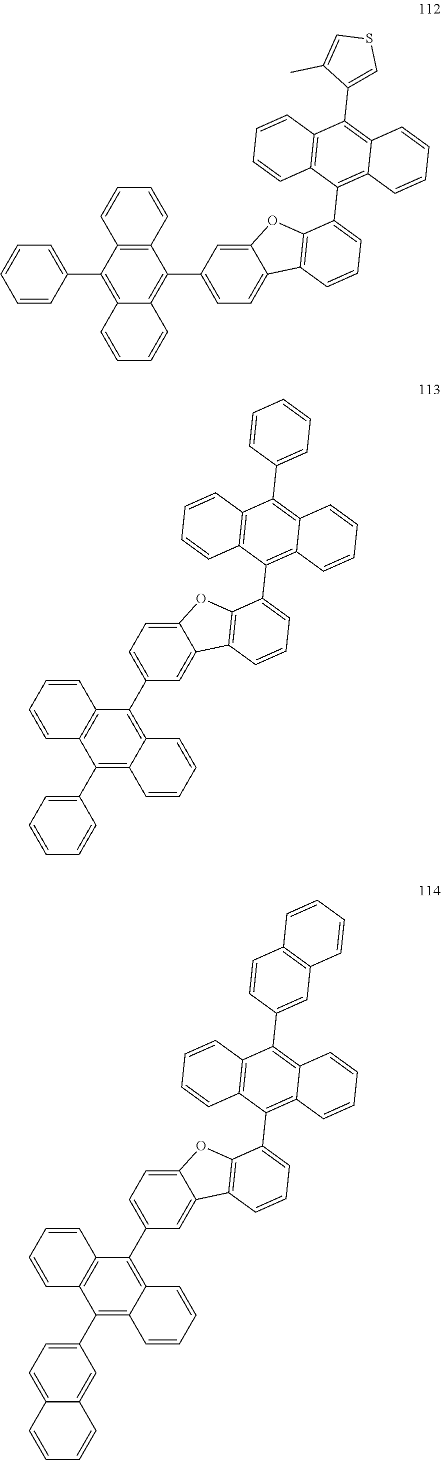 [SMILES: Cc1cscc1-c1c2ccccc2c(-c2cccc3c2oc2cc(-c4c5ccccc5c(-c5ccccc5)c5ccccc45)ccc23)c2ccccc12 ; c1ccc(-c2c3ccccc3c(-c3ccc4oc5c(-c6c7ccccc7c(-c7ccccc7)c7ccccc67)cccc5c4c3)c3ccccc23)cc1 ; c1ccc2cc(-c3c4ccccc4c(-c4ccc5oc6c(-c7c8ccccc8c(-c8ccc9ccccc9c8)c8ccccc78)cccc6c5c4)c4ccccc34)ccc2c1]

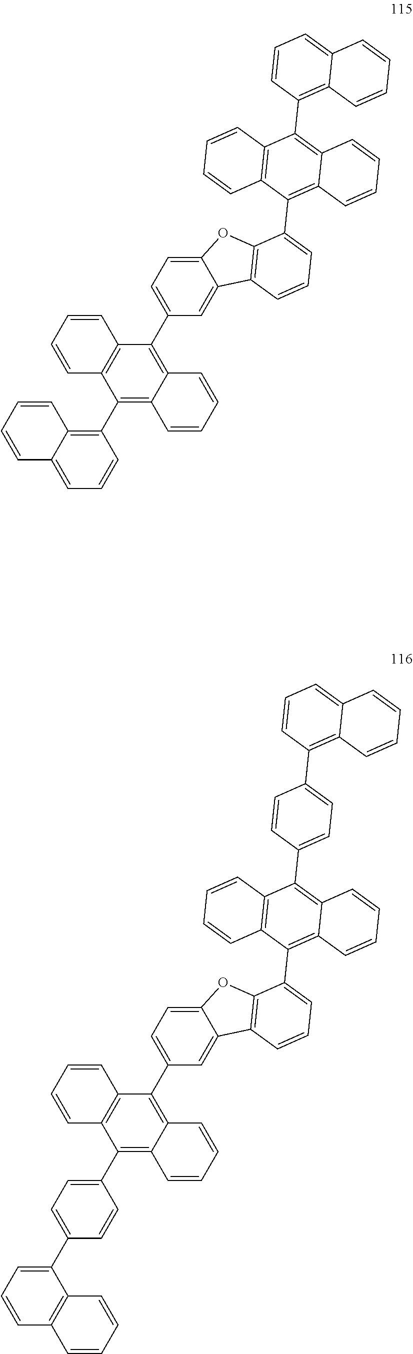 [SMILES: c1ccc2c(-c3c4ccccc4c(-c4ccc5oc6c(-c7c8ccccc8c(-c8cccc9ccccc89)c8ccccc78)cccc6c5c4)c4ccccc34)cccc2c1 ; c1ccc2c(-c3ccc(-c4c5ccccc5c(-c5ccc6oc7c(-c8c9ccccc9c(-c9ccc(-c%10cccc%11ccccc%10%11)cc9)c9ccccc89)cccc7c6c5)c5ccccc45)cc3)cccc2c1]

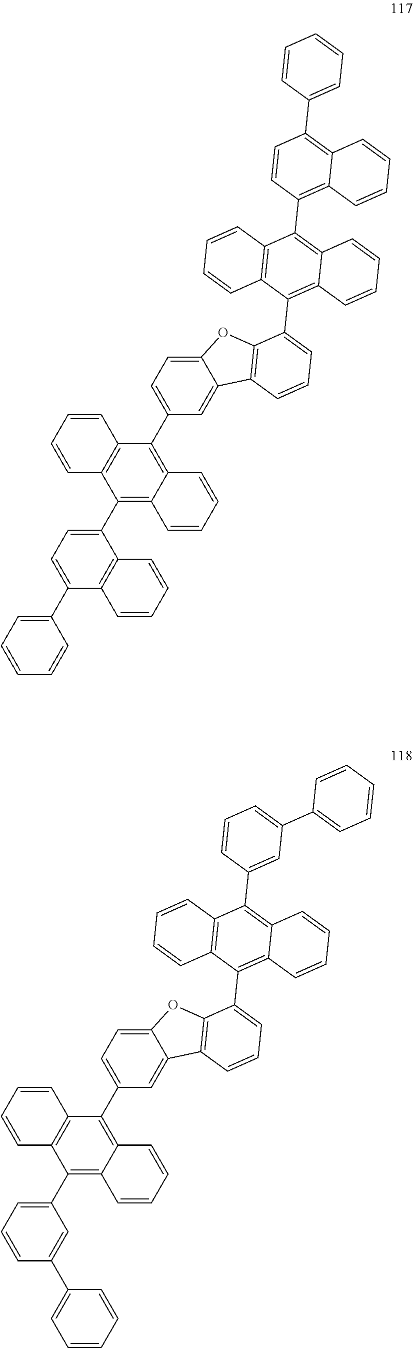 [SMILES: c1ccc(-c2ccc(-c3c4ccccc4c(-c4ccc5oc6c(-c7c8ccccc8c(-c8ccc(-c9ccccc9)c9ccccc89)c8ccccc78)cccc6c5c4)c4ccccc34)c3ccccc23)cc1 ; c1ccc(-c2cccc(-c3c4ccccc4c(-c4ccc5oc6c(-c7c8ccccc8c(-c8cccc(-c9ccccc9)c8)c8ccccc78)cccc6c5c4)c4ccccc34)c2)cc1]

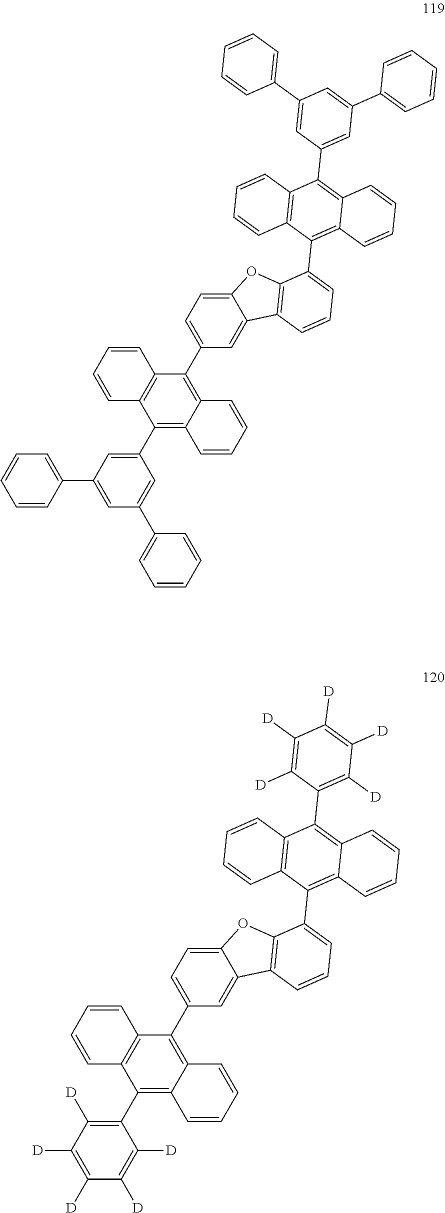 [SMILES: c1ccc(-c2cc(-c3ccccc3)cc(-c3c4ccccc4c(-c4ccc5oc6c(-c7c8ccccc8c(-c8cc(-c9ccccc9)cc(-c9ccccc9)c8)c8ccccc78)cccc6c5c4)c4ccccc34)c2)cc1 ; [2H]c1c([2H])c([2H])c(-c2c3ccccc3c(-c3ccc4oc5c(-c6c7ccccc7c(-c7c([2H])c([2H])c([2H])c([2H])c7[2H])c7ccccc67)cccc5c4c3)c3ccccc23)c([2H])c1[2H]]

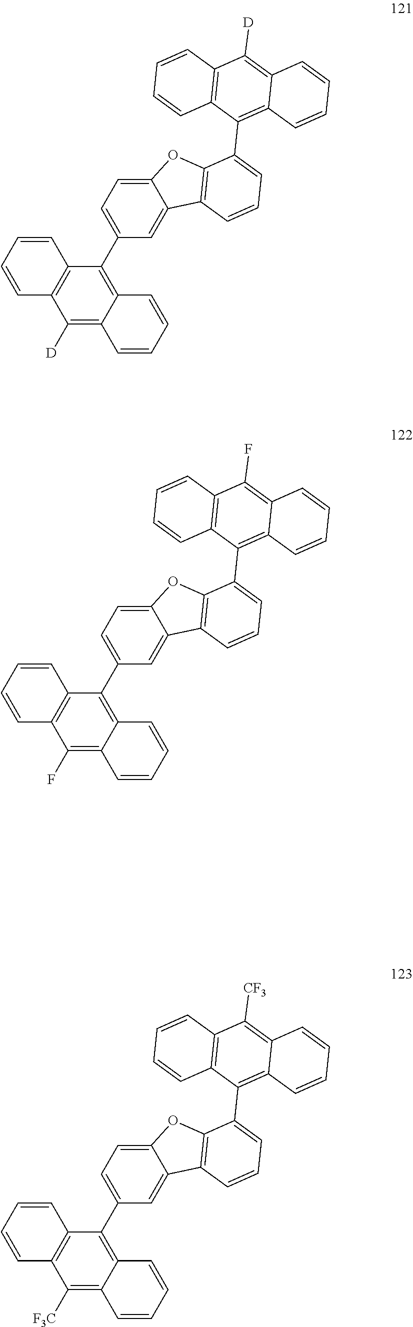 [SMILES: [2H]c1c2ccccc2c(-c2ccc3oc4c(-c5c6ccccc6c([2H])c6ccccc56)cccc4c3c2)c2ccccc12 ; Fc1c2ccccc2c(-c2ccc3oc4c(-c5c6ccccc6c(F)c6ccccc56)cccc4c3c2)c2ccccc12 ; FC(F)(F)c1c2ccccc2c(-c2ccc3oc4c(-c5c6ccccc6c(C(F)(F)F)c6ccccc56)cccc4c3c2)c2ccccc12]

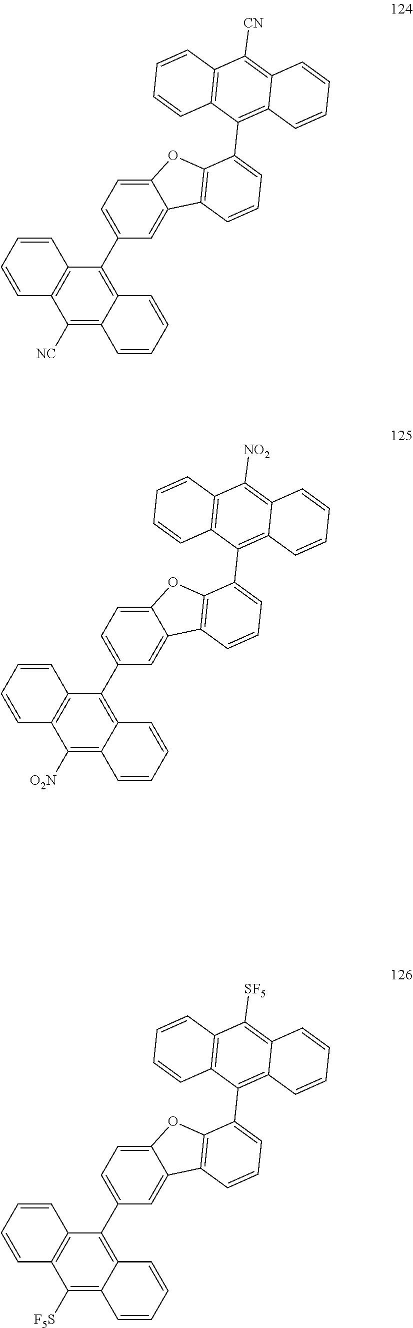

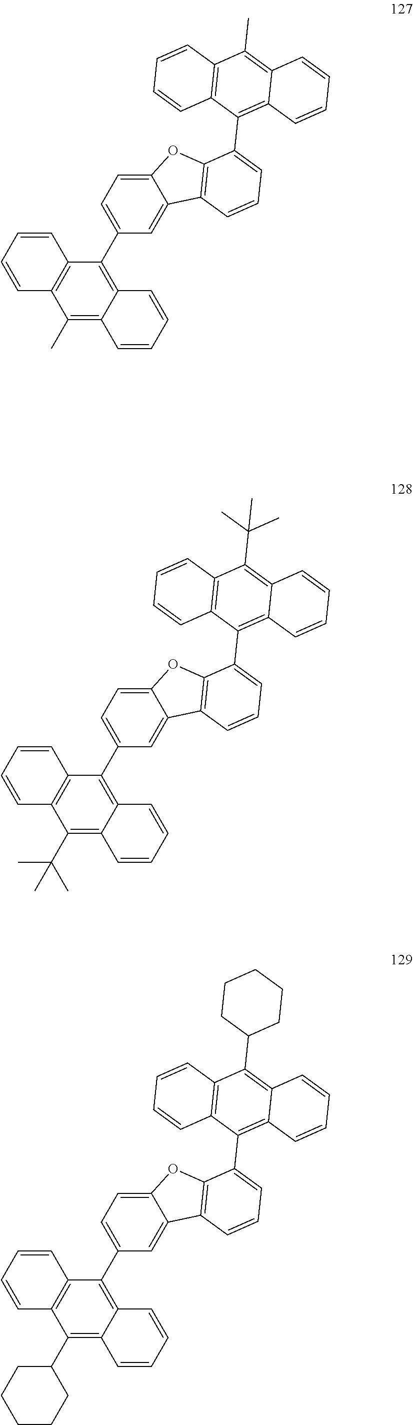 [SMILES: Cc1c2ccccc2c(-c2ccc3oc4c(-c5c6ccccc6c(C)c6ccccc56)cccc4c3c2)c2ccccc12 ; CC(C)(C)c1c2ccccc2c(-c2ccc3oc4c(-c5c6ccccc6c(C(C)(C)C)c6ccccc56)cccc4c3c2)c2ccccc12 ; c1ccc2c(C3CCCCC3)c3ccccc3c(-c3ccc4oc5c(-c6c7ccccc7c(C7CCCCC7)c7ccccc67)cccc5c4c3)c2c1]

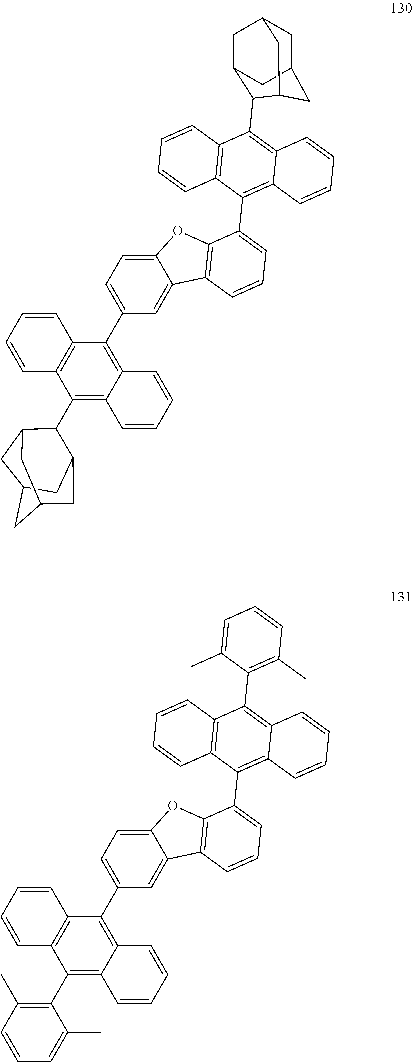 [SMILES: c1ccc2c(C3C4CC5CC(C4)CC3C5)c3ccccc3c(-c3ccc4oc5c(-c6c7ccccc7c(C7C8CC9CC(C8)CC7C9)c7ccccc67)cccc5c4c3)c2c1 ; Cc1cccc(C)c1-c1c2ccccc2c(-c2ccc3oc4c(-c5c6ccccc6c(-c6c(C)cccc6C)c6ccccc56)cccc4c3c2)c2ccccc12]

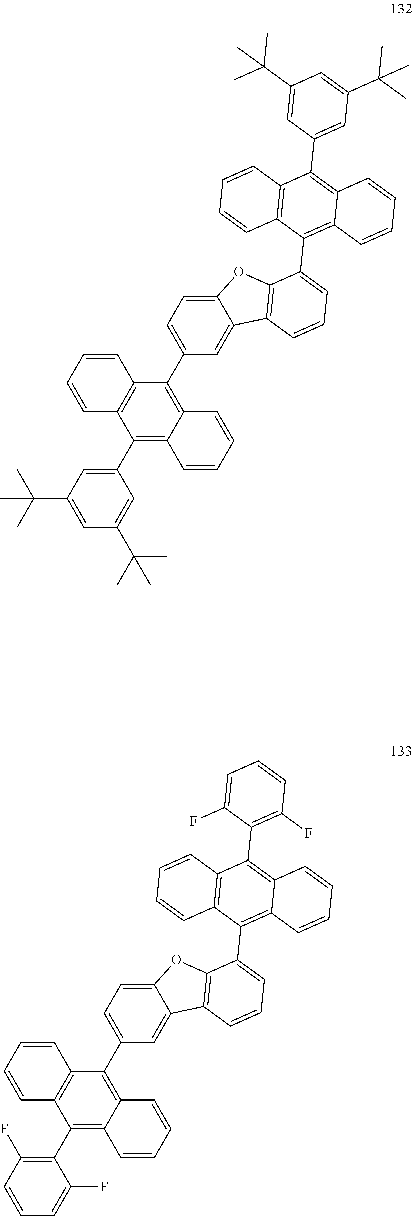 [SMILES: CC(C)(C)c1cc(-c2c3ccccc3c(-c3ccc4oc5c(-c6c7ccccc7c(-c7cc(C(C)(C)C)cc(C(C)(C)C)c7)c7ccccc67)cccc5c4c3)c3ccccc23)cc(C(C)(C)C)c1 ; Fc1cccc(F)c1-c1c2ccccc2c(-c2ccc3oc4c(-c5c6ccccc6c(-c6c(F)cccc6F)c6ccccc56)cccc4c3c2)c2ccccc12]

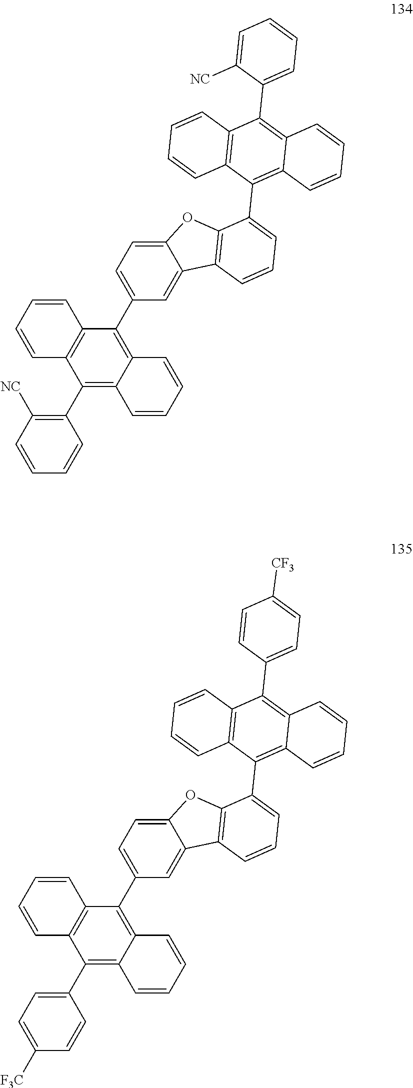 [SMILES: N#Cc1ccccc1-c1c2ccccc2c(-c2ccc3oc4c(-c5c6ccccc6c(-c6ccccc6C#N)c6ccccc56)cccc4c3c2)c2ccccc12 ; FC(F)(F)c1ccc(-c2c3ccccc3c(-c3ccc4oc5c(-c6c7ccccc7c(-c7ccc(C(F)(F)F)cc7)c7ccccc67)cccc5c4c3)c3ccccc23)cc1]

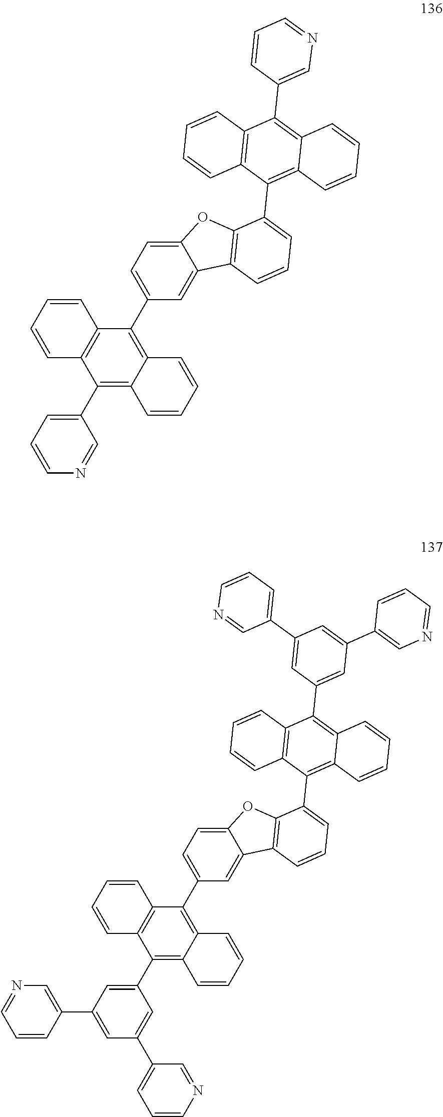 [SMILES: c1cncc(-c2c3ccccc3c(-c3ccc4oc5c(-c6c7ccccc7c(-c7cccnc7)c7ccccc67)cccc5c4c3)c3ccccc23)c1 ; c1cncc(-c2cc(-c3cccnc3)cc(-c3c4ccccc4c(-c4ccc5oc6c(-c7c8ccccc8c(-c8cc(-c9cccnc9)cc(-c9cccnc9)c8)c8ccccc78)cccc6c5c4)c4ccccc34)c2)c1]

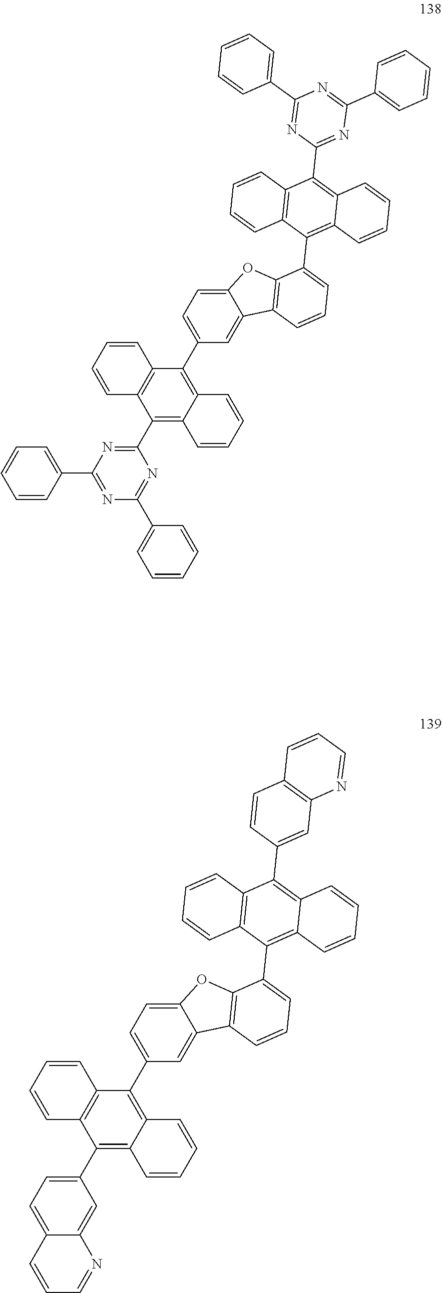 [SMILES: c1ccc(-c2nc(-c3ccccc3)nc(-c3c4ccccc4c(-c4ccc5oc6c(-c7c8ccccc8c(-c8nc(-c9ccccc9)nc(-c9ccccc9)n8)c8ccccc78)cccc6c5c4)c4ccccc34)n2)cc1 ; c1cnc2cc(-c3c4ccccc4c(-c4ccc5oc6c(-c7c8ccccc8c(-c8ccc9cccnc9c8)c8ccccc78)cccc6c5c4)c4ccccc34)ccc2c1]

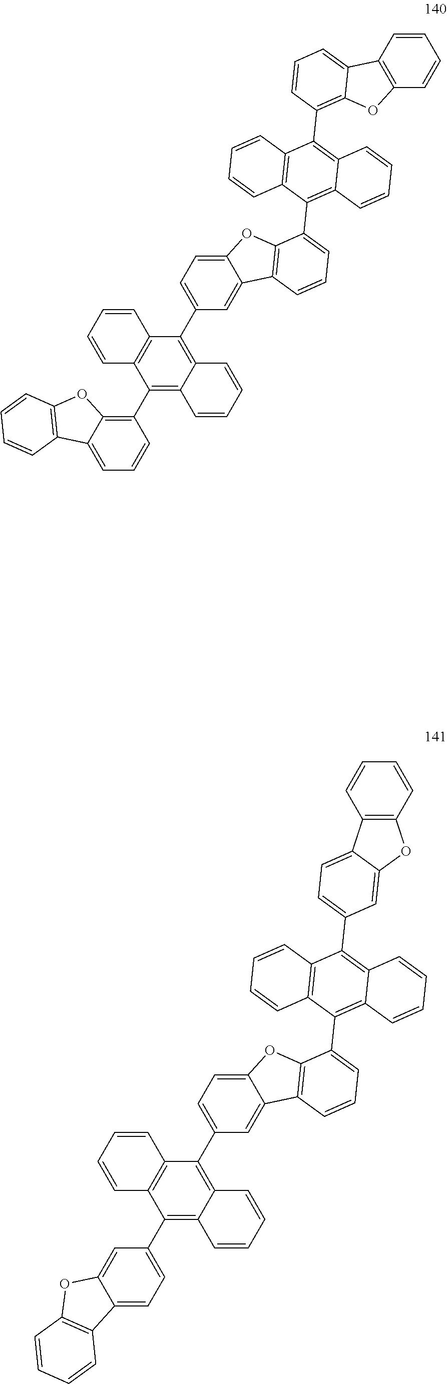 [SMILES: c1ccc2c(c1)oc1c(-c3c4ccccc4c(-c4ccc5oc6c(-c7c8ccccc8c(-c8cccc9c8oc8ccccc89)c8ccccc78)cccc6c5c4)c4ccccc34)cccc12 ; c1ccc2c(c1)oc1cc(-c3c4ccccc4c(-c4ccc5oc6c(-c7c8ccccc8c(-c8ccc9c(c8)oc8ccccc89)c8ccccc78)cccc6c5c4)c4ccccc34)ccc12]

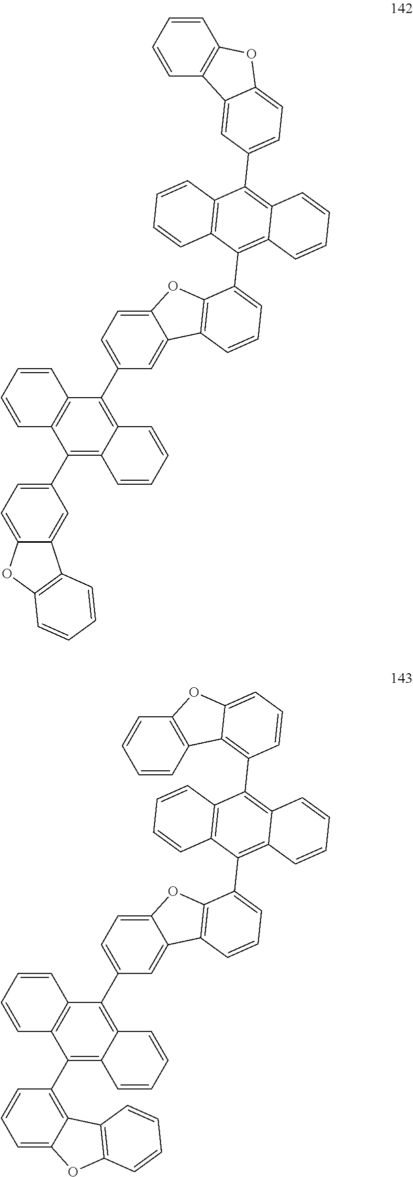 [SMILES: c1ccc2c(c1)oc1ccc(-c3c4ccccc4c(-c4ccc5oc6c(-c7c8ccccc8c(-c8ccc9oc%10ccccc%10c9c8)c8ccccc78)cccc6c5c4)c4ccccc34)cc12 ; c1ccc2c(c1)oc1cccc(-c3c4ccccc4c(-c4ccc5oc6c(-c7c8ccccc8c(-c8cccc9oc%10ccccc%10c89)c8ccccc78)cccc6c5c4)c4ccccc34)c12]

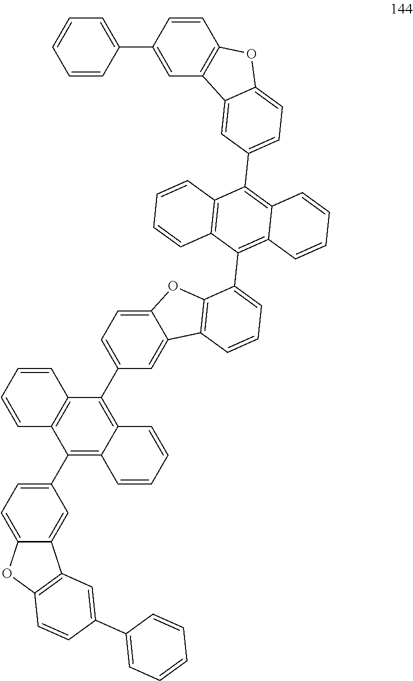 [SMILES: c1ccc(-c2ccc3oc4ccc(-c5c6ccccc6c(-c6ccc7oc8c(-c9c%10ccccc%10c(-c%10ccc%11oc%12ccc(-c%13ccccc%13)cc%12c%11c%10)c%10ccccc9%10)cccc8c7c6)c6ccccc56)cc4c3c2)cc1]

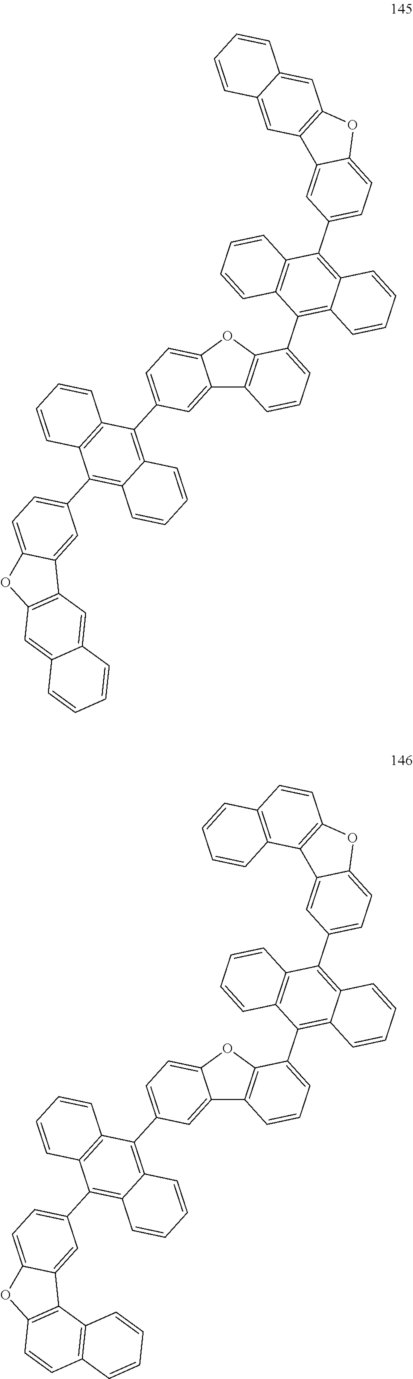 [SMILES: c1ccc2cc3c(cc2c1)oc1ccc(-c2c4ccccc4c(-c4ccc5oc6c(-c7c8ccccc8c(-c8ccc9oc%10cc%11ccccc%11cc%10c9c8)c8ccccc78)cccc6c5c4)c4ccccc24)cc13 ; c1ccc2c(c1)ccc1oc3ccc(-c4c5ccccc5c(-c5ccc6oc7c(-c8c9ccccc9c(-c9ccc%10oc%11ccc%12ccccc%12c%11c%10c9)c9ccccc89)cccc7c6c5)c5ccccc45)cc3c12]

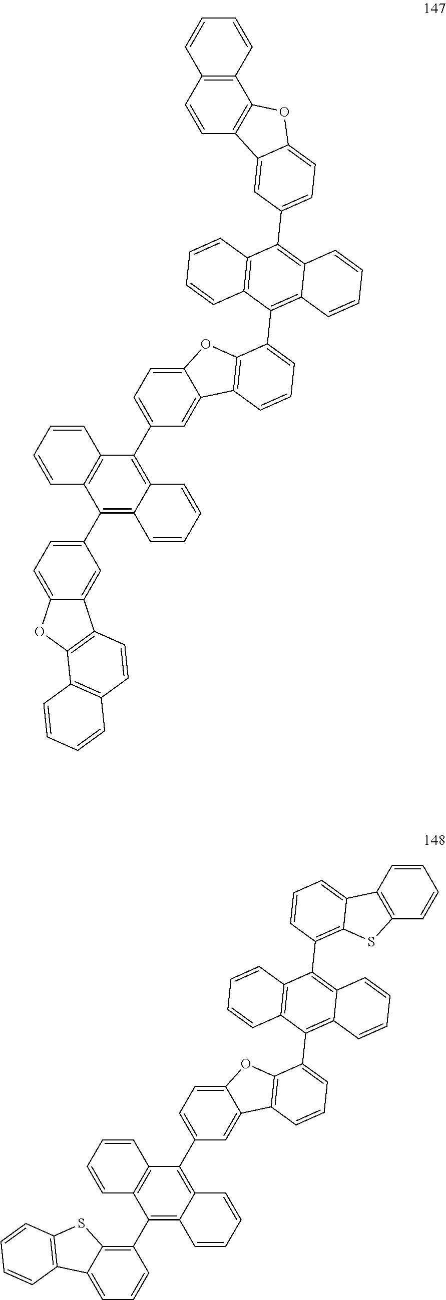 [SMILES: c1ccc2c(c1)ccc1c3cc(-c4c5ccccc5c(-c5ccc6oc7c(-c8c9ccccc9c(-c9ccc%10oc%11c%12ccccc%12ccc%11c%10c9)c9ccccc89)cccc7c6c5)c5ccccc45)ccc3oc21 ; c1ccc2c(c1)sc1c(-c3c4ccccc4c(-c4ccc5oc6c(-c7c8ccccc8c(-c8cccc9c8sc8ccccc89)c8ccccc78)cccc6c5c4)c4ccccc34)cccc12]

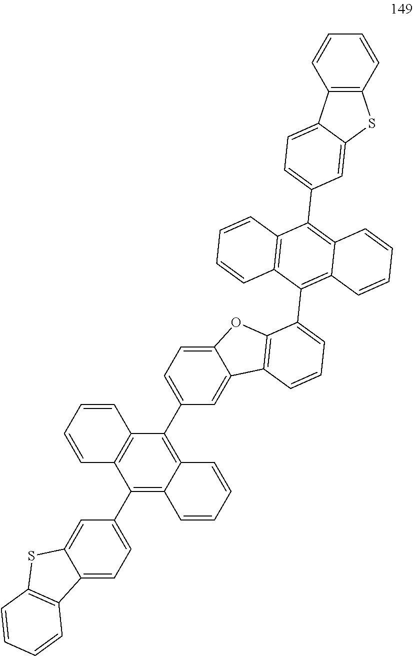 [SMILES: c1ccc2c(c1)sc1cc(-c3c4ccccc4c(-c4ccc5oc6c(-c7c8ccccc8c(-c8ccc9c(c8)sc8ccccc89)c8ccccc78)cccc6c5c4)c4ccccc34)ccc12]

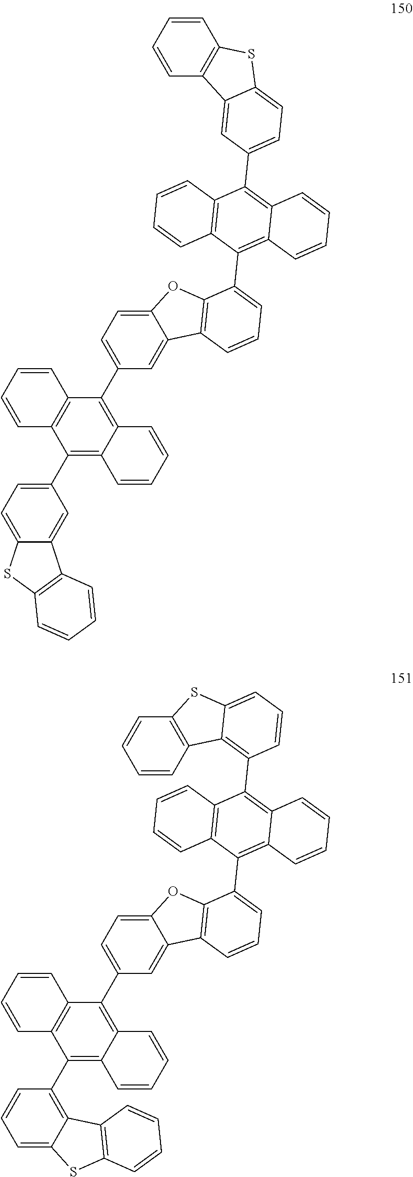 [SMILES: c1ccc2c(c1)sc1ccc(-c3c4ccccc4c(-c4ccc5oc6c(-c7c8ccccc8c(-c8ccc9sc%10ccccc%10c9c8)c8ccccc78)cccc6c5c4)c4ccccc34)cc12 ; c1ccc2c(c1)sc1cccc(-c3c4ccccc4c(-c4ccc5oc6c(-c7c8ccccc8c(-c8cccc9sc%10ccccc%10c89)c8ccccc78)cccc6c5c4)c4ccccc34)c12]

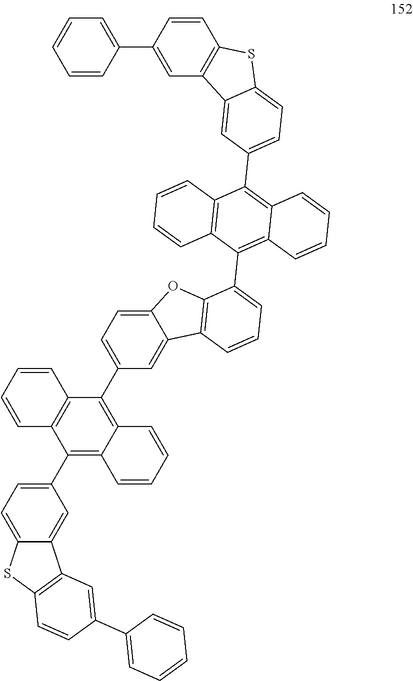 [SMILES: c1ccc(-c2ccc3sc4ccc(-c5c6ccccc6c(-c6ccc7oc8c(-c9c%10ccccc%10c(-c%10ccc%11sc%12ccc(-c%13ccccc%13)cc%12c%11c%10)c%10ccccc9%10)cccc8c7c6)c6ccccc56)cc4c3c2)cc1]

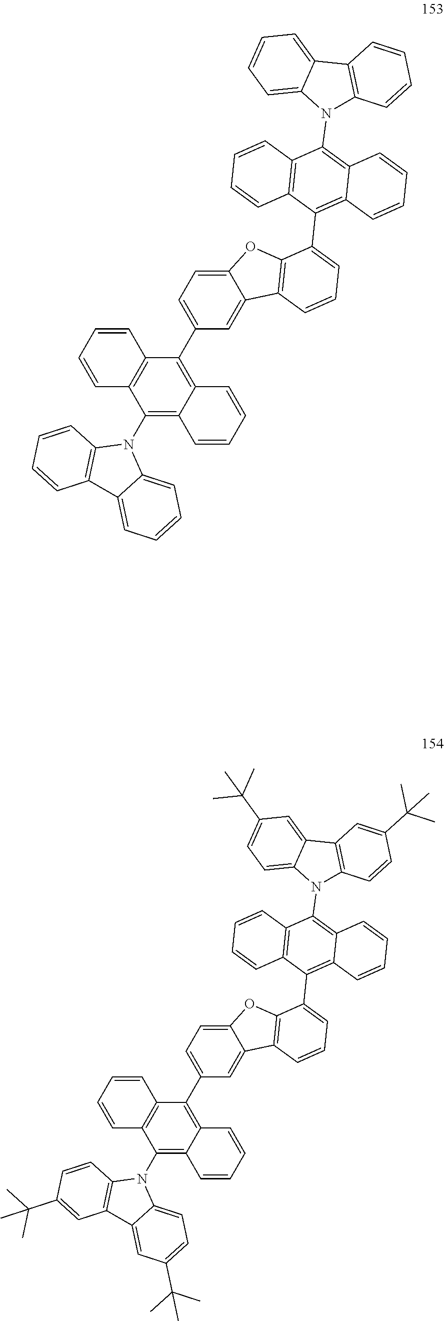 [SMILES: c1ccc2c(-n3c4ccccc4c4ccccc43)c3ccccc3c(-c3ccc4oc5c(-c6c7ccccc7c(-n7c8ccccc8c8ccccc87)c7ccccc67)cccc5c4c3)c2c1 ; CC(C)(C)c1ccc2c(c1)c1cc(C(C)(C)C)ccc1n2-c1c2ccccc2c(-c2ccc3oc4c(-c5c6ccccc6c(-n6c7ccc(C(C)(C)C)cc7c7cc(C(C)(C)C)ccc76)c6ccccc56)cccc4c3c2)c2ccccc12]

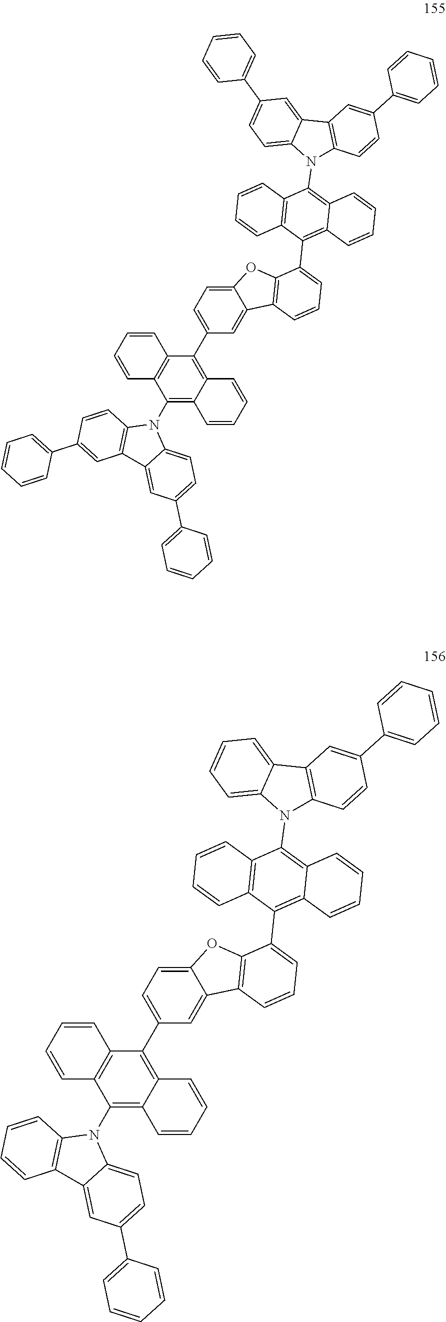 [SMILES: c1ccc(-c2ccc3c(c2)c2cc(-c4ccccc4)ccc2n3-c2c3ccccc3c(-c3ccc4oc5c(-c6c7ccccc7c(-n7c8ccc(-c9ccccc9)cc8c8cc(-c9ccccc9)ccc87)c7ccccc67)cccc5c4c3)c3ccccc23)cc1 ; c1ccc(-c2ccc3c(c2)c2ccccc2n3-c2c3ccccc3c(-c3ccc4oc5c(-c6c7ccccc7c(-n7c8ccccc8c8cc(-c9ccccc9)ccc87)c7ccccc67)cccc5c4c3)c3ccccc23)cc1]

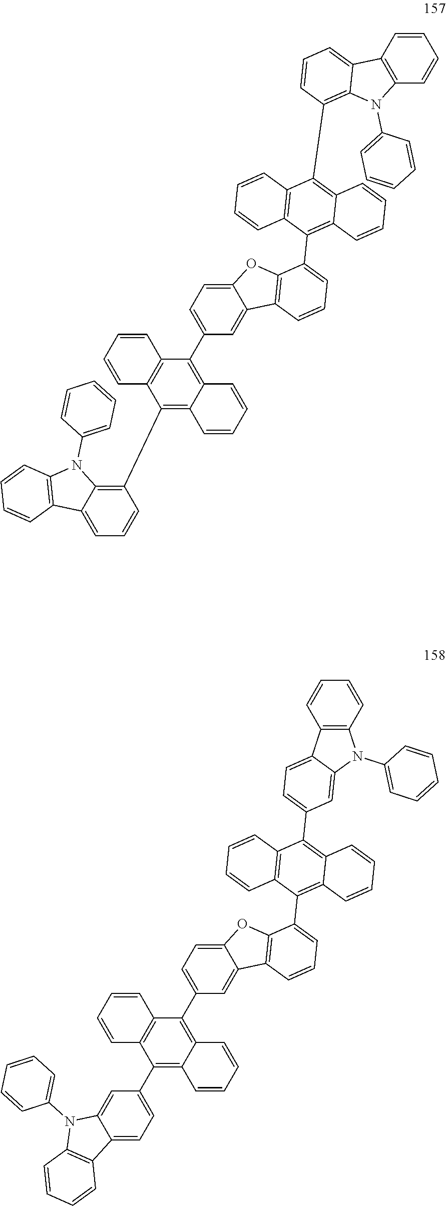 [SMILES: c1ccc(-n2c3ccccc3c3cccc(-c4c5ccccc5c(-c5ccc6oc7c(-c8c9ccccc9c(-c9cccc%10c%11ccccc%11n(-c%11ccccc%11)c9%10)c9ccccc89)cccc7c6c5)c5ccccc45)c32)cc1 ; c1ccc(-n2c3ccccc3c3ccc(-c4c5ccccc5c(-c5ccc6oc7c(-c8c9ccccc9c(-c9ccc%10c%11ccccc%11n(-c%11ccccc%11)c%10c9)c9ccccc89)cccc7c6c5)c5ccccc45)cc32)cc1]

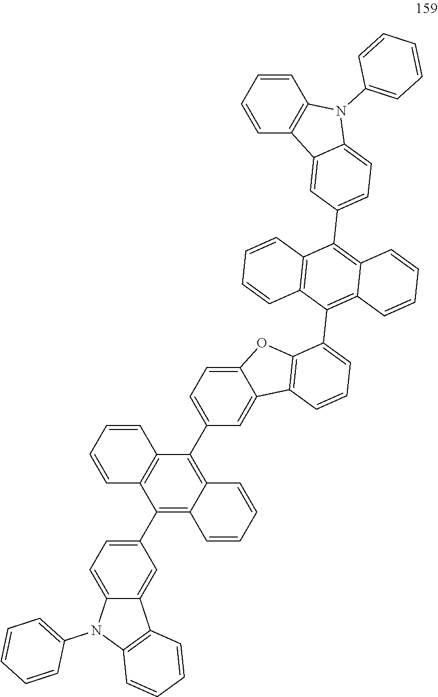 [SMILES: c1ccc(-n2c3ccccc3c3cc(-c4c5ccccc5c(-c5ccc6oc7c(-c8c9ccccc9c(-c9ccc%10c(c9)c9ccccc9n%10-c9ccccc9)c9ccccc89)cccc7c6c5)c5ccccc45)ccc32)cc1]

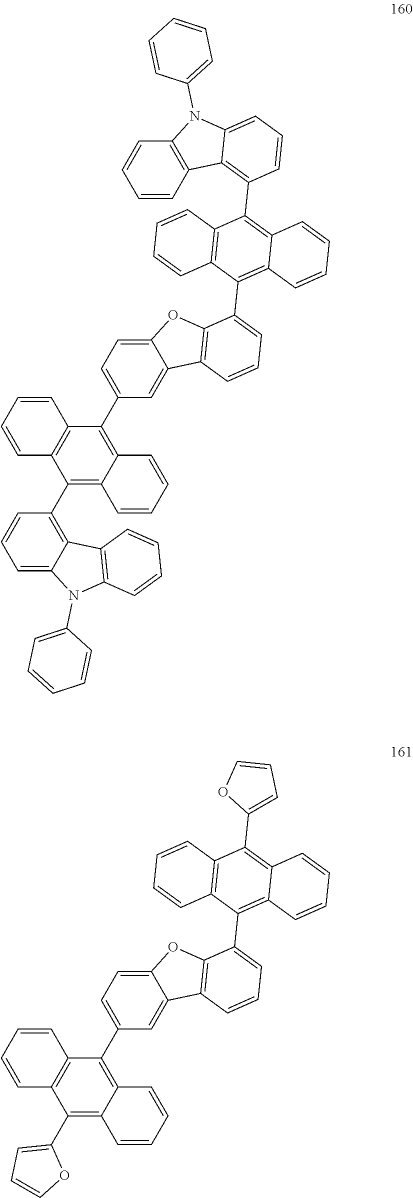 [SMILES: c1ccc(-n2c3ccccc3c3c(-c4c5ccccc5c(-c5ccc6oc7c(-c8c9ccccc9c(-c9cccc%10c9c9ccccc9n%10-c9ccccc9)c9ccccc89)cccc7c6c5)c5ccccc45)cccc32)cc1 ; c1coc(-c2c3ccccc3c(-c3ccc4oc5c(-c6c7ccccc7c(-c7ccco7)c7ccccc67)cccc5c4c3)c3ccccc23)c1]

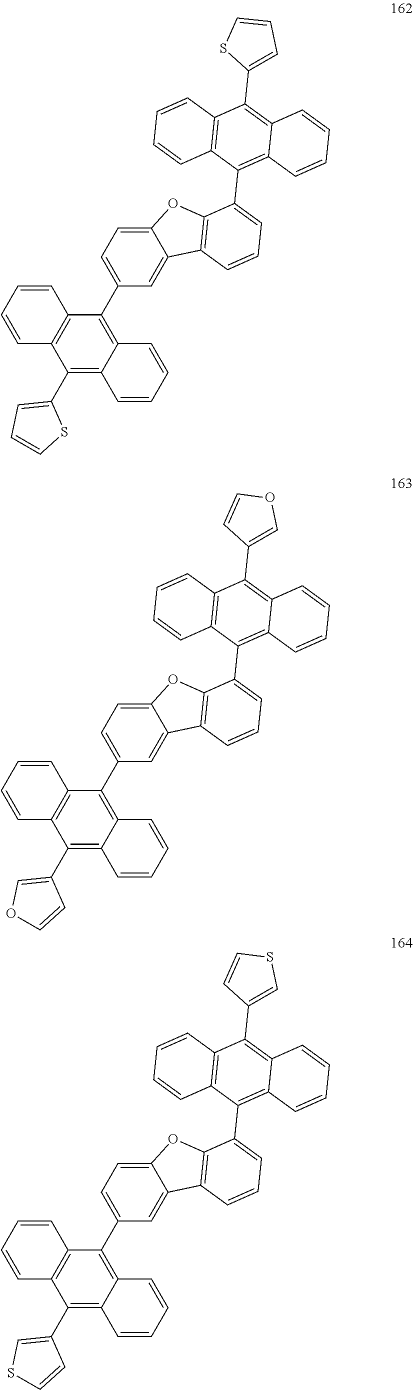 [SMILES: c1csc(-c2c3ccccc3c(-c3ccc4oc5c(-c6c7ccccc7c(-c7cccs7)c7ccccc67)cccc5c4c3)c3ccccc23)c1 ; c1ccc2c(-c3ccc4oc5c(-c6c7ccccc7c(-c7ccoc7)c7ccccc67)cccc5c4c3)c3ccccc3c(-c3ccoc3)c2c1 ; c1ccc2c(-c3ccc4oc5c(-c6c7ccccc7c(-c7ccsc7)c7ccccc67)cccc5c4c3)c3ccccc3c(-c3ccsc3)c2c1]

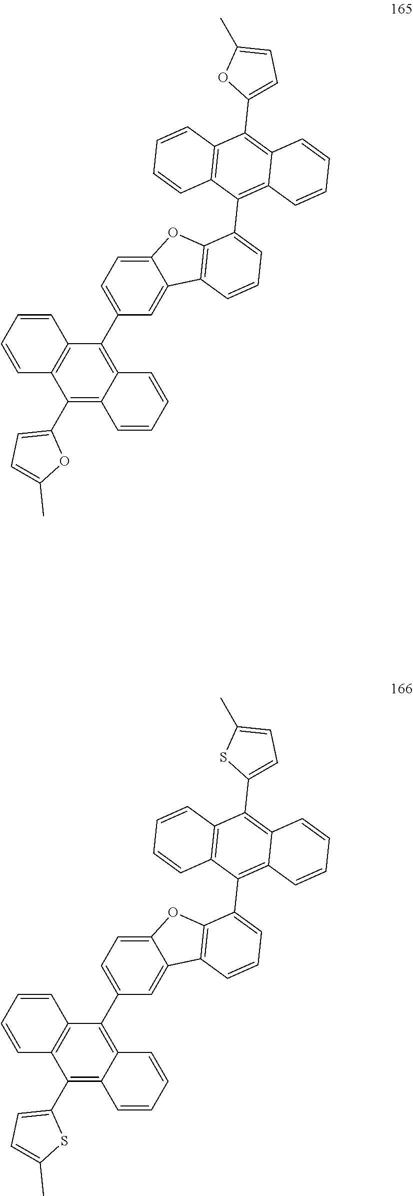 [SMILES: Cc1ccc(-c2c3ccccc3c(-c3ccc4oc5c(-c6c7ccccc7c(-c7ccc(C)o7)c7ccccc67)cccc5c4c3)c3ccccc23)o1 ; Cc1ccc(-c2c3ccccc3c(-c3ccc4oc5c(-c6c7ccccc7c(-c7ccc(C)s7)c7ccccc67)cccc5c4c3)c3ccccc23)s1]

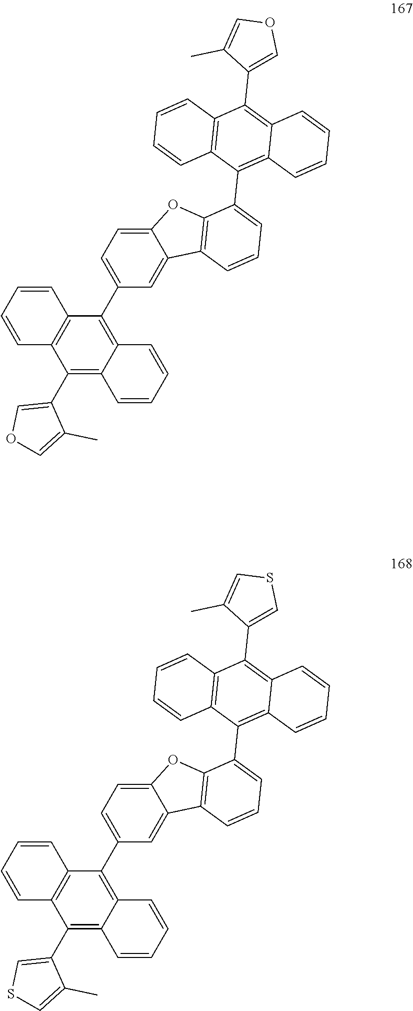 [SMILES: Cc1cocc1-c1c2ccccc2c(-c2ccc3oc4c(-c5c6ccccc6c(-c6cocc6C)c6ccccc56)cccc4c3c2)c2ccccc12 ; Cc1cscc1-c1c2ccccc2c(-c2ccc3oc4c(-c5c6ccccc6c(-c6cscc6C)c6ccccc56)cccc4c3c2)c2ccccc12]

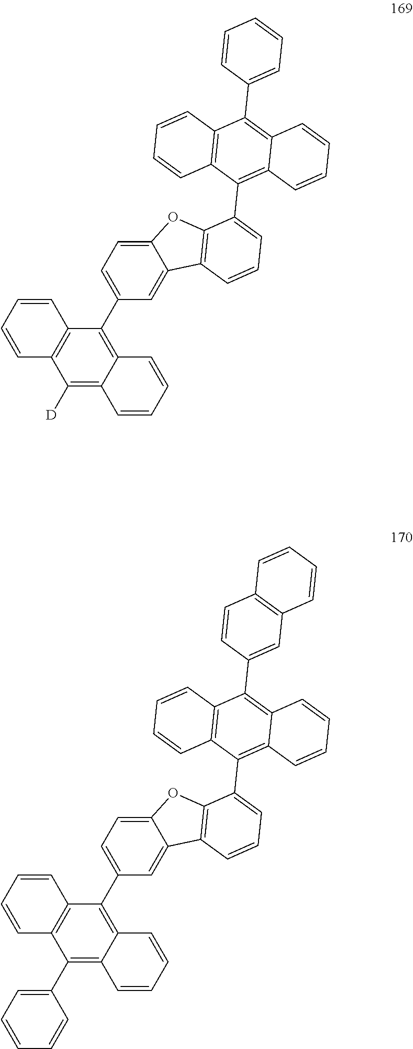 [SMILES: [2H]c1c2ccccc2c(-c2ccc3oc4c(-c5c6ccccc6c(-c6ccccc6)c6ccccc56)cccc4c3c2)c2ccccc12 ; c1ccc(-c2c3ccccc3c(-c3ccc4oc5c(-c6c7ccccc7c(-c7ccc8ccccc8c7)c7ccccc67)cccc5c4c3)c3ccccc23)cc1]

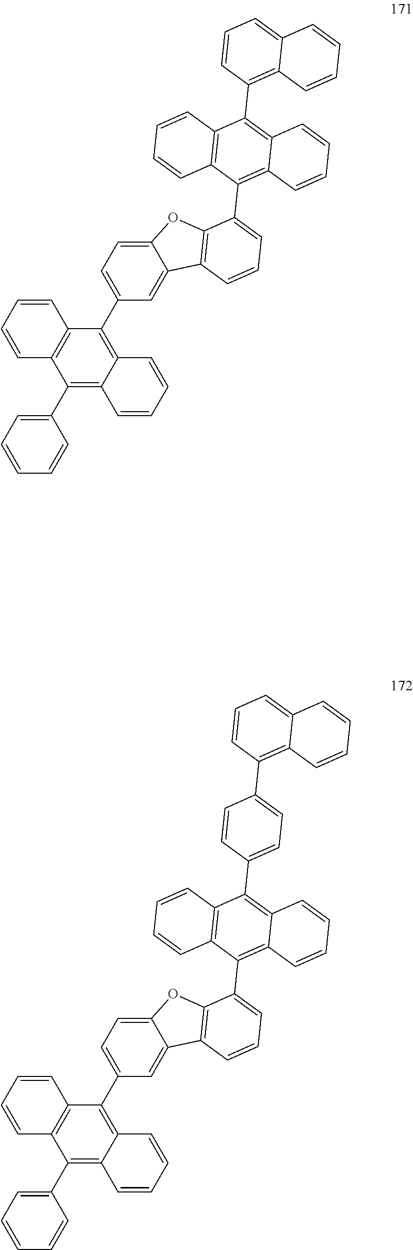 [SMILES: c1ccc(-c2c3ccccc3c(-c3ccc4oc5c(-c6c7ccccc7c(-c7cccc8ccccc78)c7ccccc67)cccc5c4c3)c3ccccc23)cc1 ; c1ccc(-c2c3ccccc3c(-c3ccc4oc5c(-c6c7ccccc7c(-c7ccc(-c8cccc9ccccc89)cc7)c7ccccc67)cccc5c4c3)c3ccccc23)cc1]

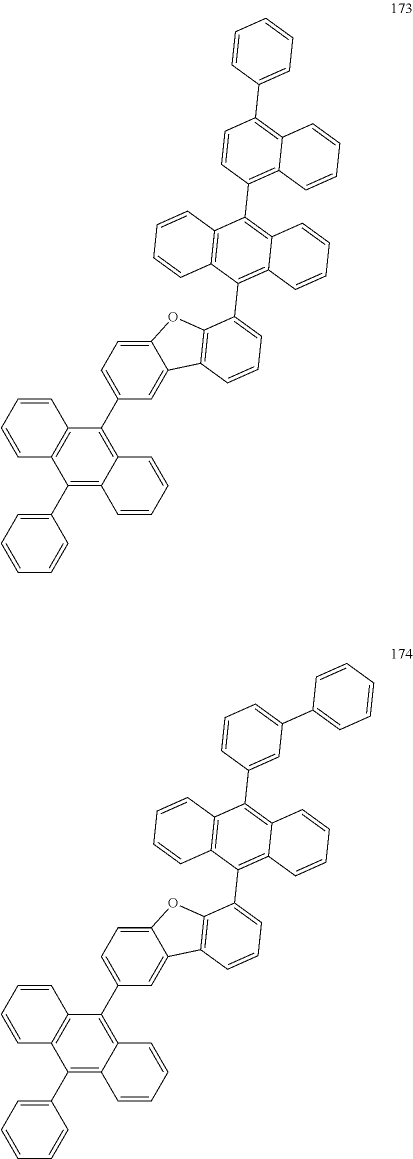 [SMILES: c1ccc(-c2ccc(-c3c4ccccc4c(-c4cccc5c4oc4ccc(-c6c7ccccc7c(-c7ccccc7)c7ccccc67)cc45)c4ccccc34)c3ccccc23)cc1 ; c1ccc(-c2cccc(-c3c4ccccc4c(-c4cccc5c4oc4ccc(-c6c7ccccc7c(-c7ccccc7)c7ccccc67)cc45)c4ccccc34)c2)cc1]

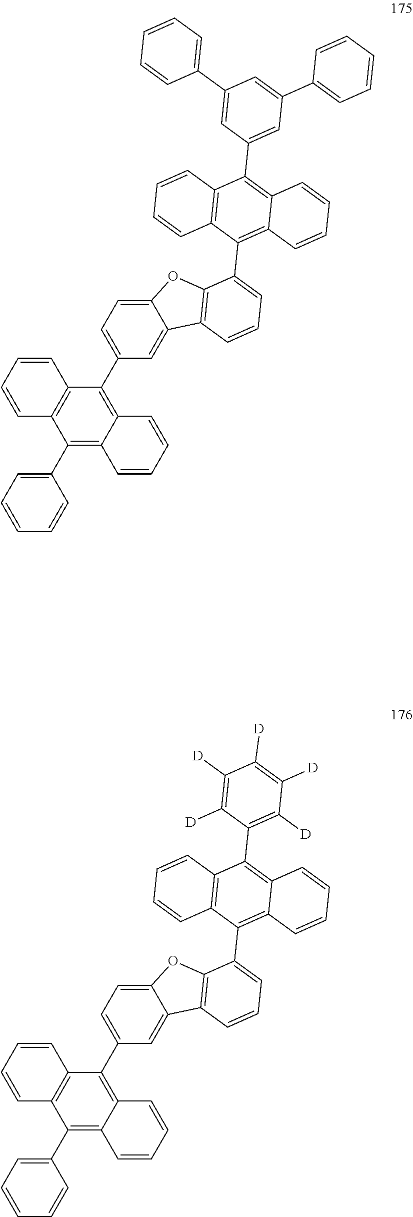 [SMILES: c1ccc(-c2cc(-c3ccccc3)cc(-c3c4ccccc4c(-c4cccc5c4oc4ccc(-c6c7ccccc7c(-c7ccccc7)c7ccccc67)cc45)c4ccccc34)c2)cc1 ; [2H]c1c([2H])c([2H])c(-c2c3ccccc3c(-c3cccc4c3oc3ccc(-c5c6ccccc6c(-c6ccccc6)c6ccccc56)cc34)c3ccccc23)c([2H])c1[2H]]

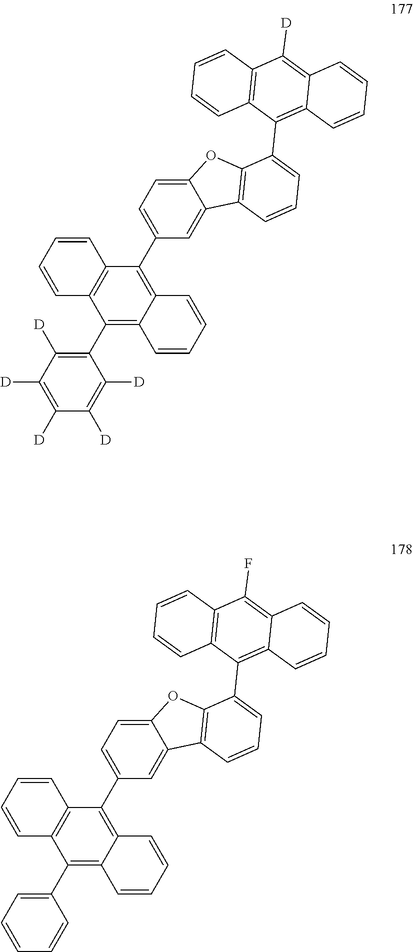 [SMILES: [2H]c1c([2H])c([2H])c(-c2c3ccccc3c(-c3ccc4oc5c(-c6c7ccccc7c([2H])c7ccccc67)cccc5c4c3)c3ccccc23)c([2H])c1[2H] ; Fc1c2ccccc2c(-c2cccc3c2oc2ccc(-c4c5ccccc5c(-c5ccccc5)c5ccccc45)cc23)c2ccccc12]

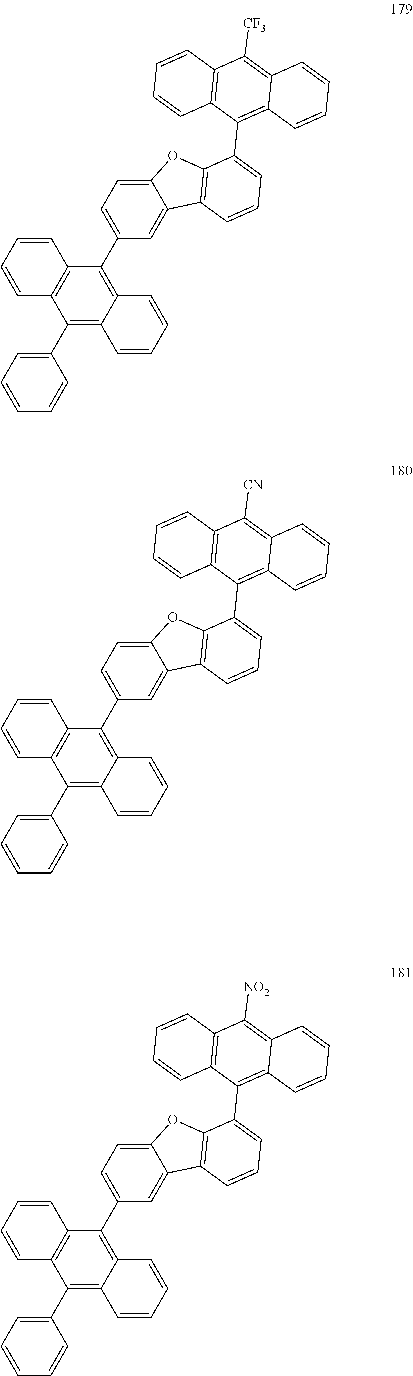 [SMILES: FC(F)(F)c1c2ccccc2c(-c2cccc3c2oc2ccc(-c4c5ccccc5c(-c5ccccc5)c5ccccc45)cc23)c2ccccc12 ; N#Cc1c2ccccc2c(-c2cccc3c2oc2ccc(-c4c5ccccc5c(-c5ccccc5)c5ccccc45)cc23)c2ccccc12 ; O=[N+]([O-])c1c2ccccc2c(-c2cccc3c2oc2ccc(-c4c5ccccc5c(-c5ccccc5)c5ccccc45)cc23)c2ccccc12]

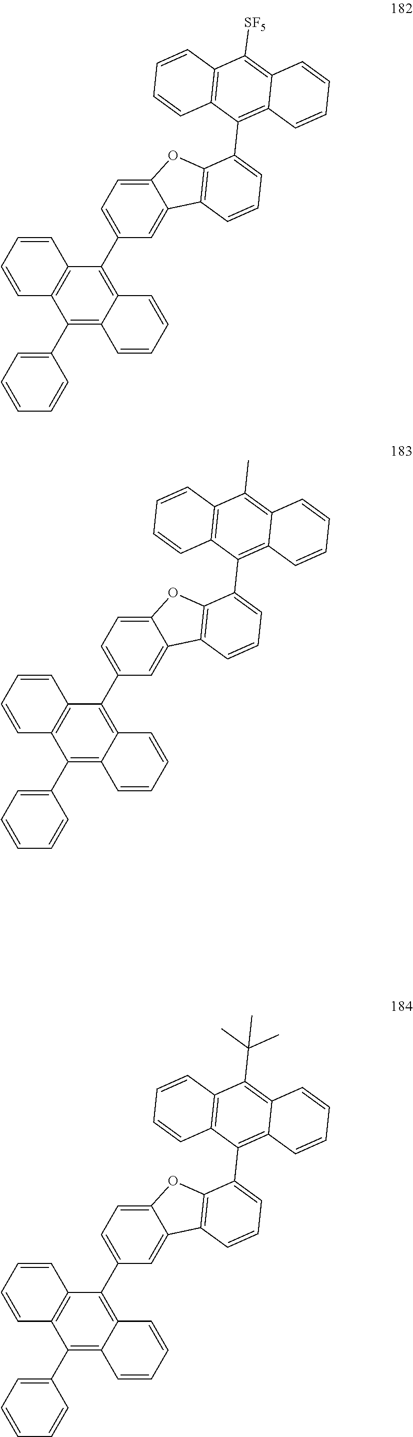

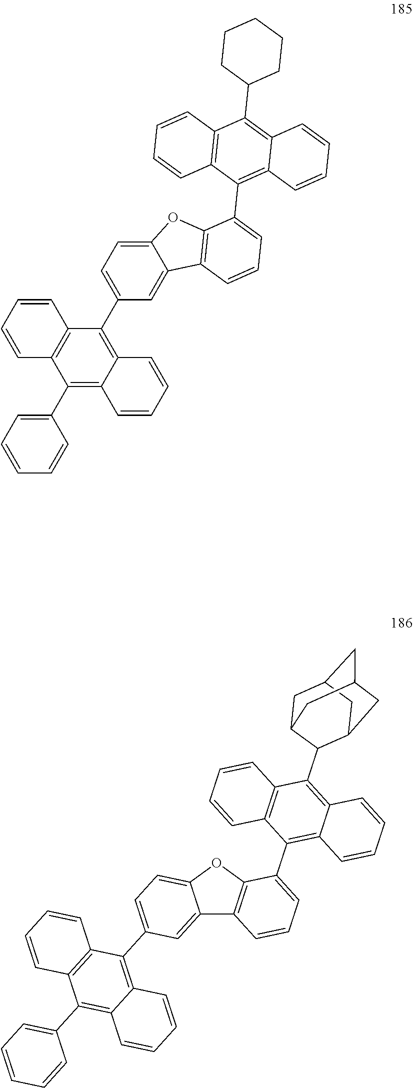 [SMILES: c1ccc(-c2c3ccccc3c(-c3ccc4oc5c(-c6c7ccccc7c(C7CCCCC7)c7ccccc67)cccc5c4c3)c3ccccc23)cc1 ; c1ccc(-c2c3ccccc3c(-c3ccc4oc5c(-c6c7ccccc7c(C7C8CC9CC(C8)CC7C9)c7ccccc67)cccc5c4c3)c3ccccc23)cc1]

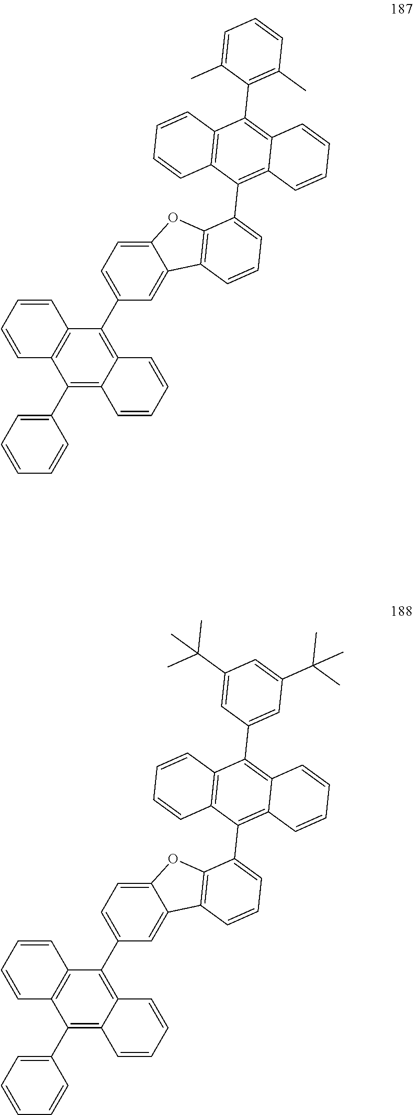 [SMILES: Cc1cccc(C)c1-c1c2ccccc2c(-c2cccc3c2oc2ccc(-c4c5ccccc5c(-c5ccccc5)c5ccccc45)cc23)c2ccccc12 ; CC(C)(C)c1cc(-c2c3ccccc3c(-c3cccc4c3oc3ccc(-c5c6ccccc6c(-c6ccccc6)c6ccccc56)cc34)c3ccccc23)cc(C(C)(C)C)c1]

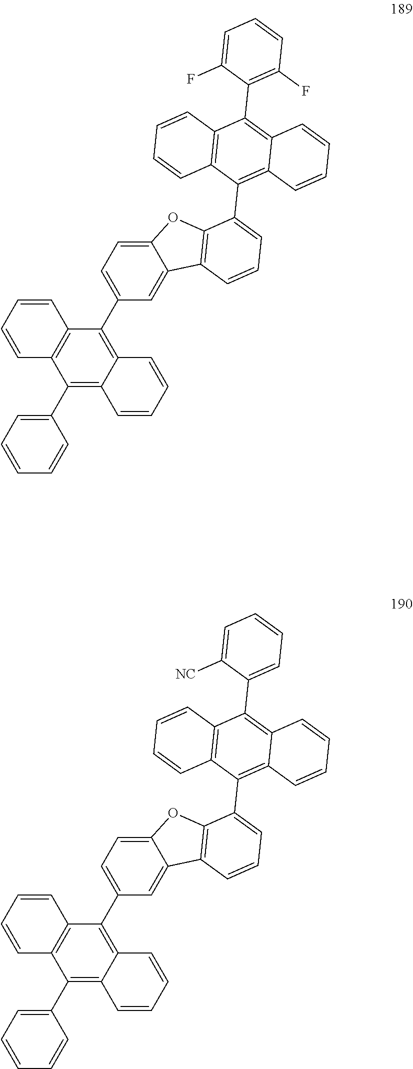 [SMILES: Fc1cccc(F)c1-c1c2ccccc2c(-c2cccc3c2oc2ccc(-c4c5ccccc5c(-c5ccccc5)c5ccccc45)cc23)c2ccccc12 ; N#Cc1ccccc1-c1c2ccccc2c(-c2cccc3c2oc2ccc(-c4c5ccccc5c(-c5ccccc5)c5ccccc45)cc23)c2ccccc12]

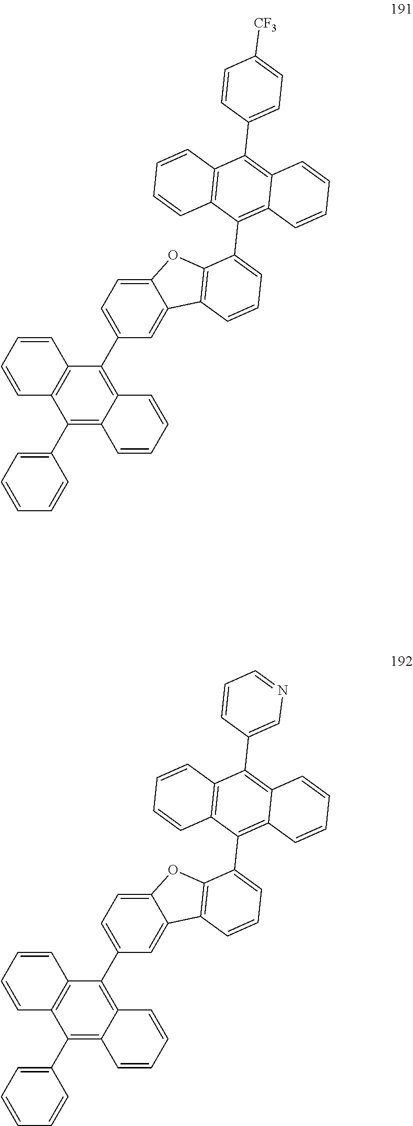 [SMILES: FC(F)(F)c1ccc(-c2c3ccccc3c(-c3cccc4c3oc3ccc(-c5c6ccccc6c(-c6ccccc6)c6ccccc56)cc34)c3ccccc23)cc1 ; c1ccc(-c2c3ccccc3c(-c3ccc4oc5c(-c6c7ccccc7c(-c7cccnc7)c7ccccc67)cccc5c4c3)c3ccccc23)cc1]

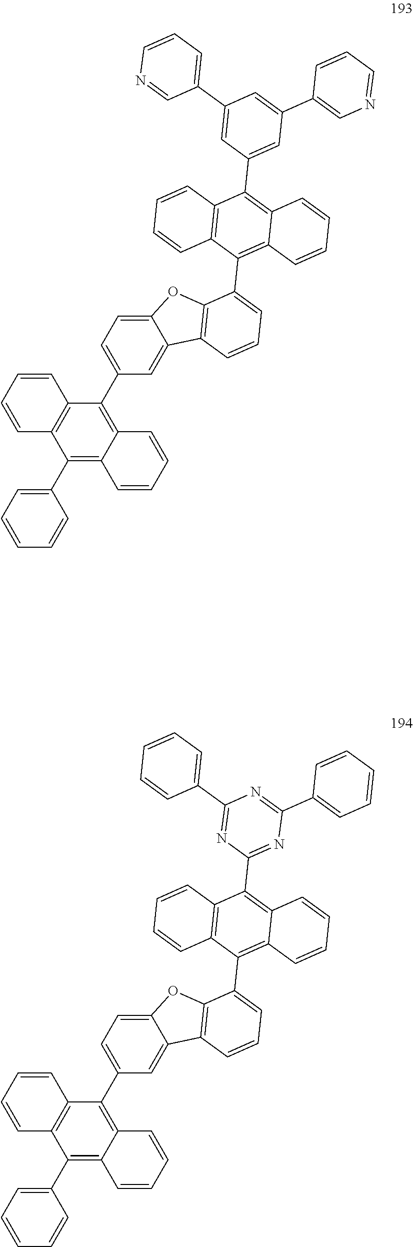 [SMILES: c1ccc(-c2c3ccccc3c(-c3ccc4oc5c(-c6c7ccccc7c(-c7cc(-c8cccnc8)cc(-c8cccnc8)c7)c7ccccc67)cccc5c4c3)c3ccccc23)cc1 ; c1ccc(-c2nc(-c3ccccc3)nc(-c3c4ccccc4c(-c4cccc5c4oc4ccc(-c6c7ccccc7c(-c7ccccc7)c7ccccc67)cc45)c4ccccc34)n2)cc1]

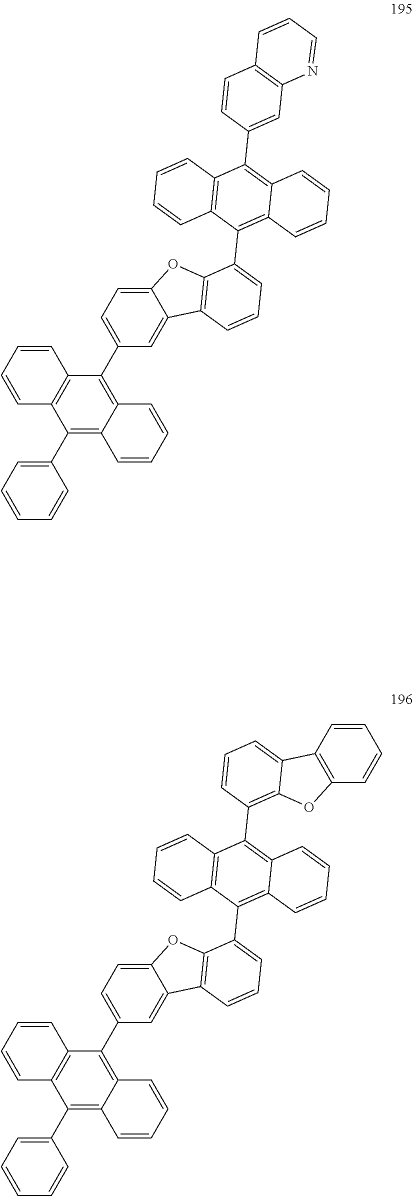 [SMILES: c1ccc(-c2c3ccccc3c(-c3ccc4oc5c(-c6c7ccccc7c(-c7ccc8cccnc8c7)c7ccccc67)cccc5c4c3)c3ccccc23)cc1 ; c1ccc(-c2c3ccccc3c(-c3ccc4oc5c(-c6c7ccccc7c(-c7cccc8c7oc7ccccc78)c7ccccc67)cccc5c4c3)c3ccccc23)cc1]

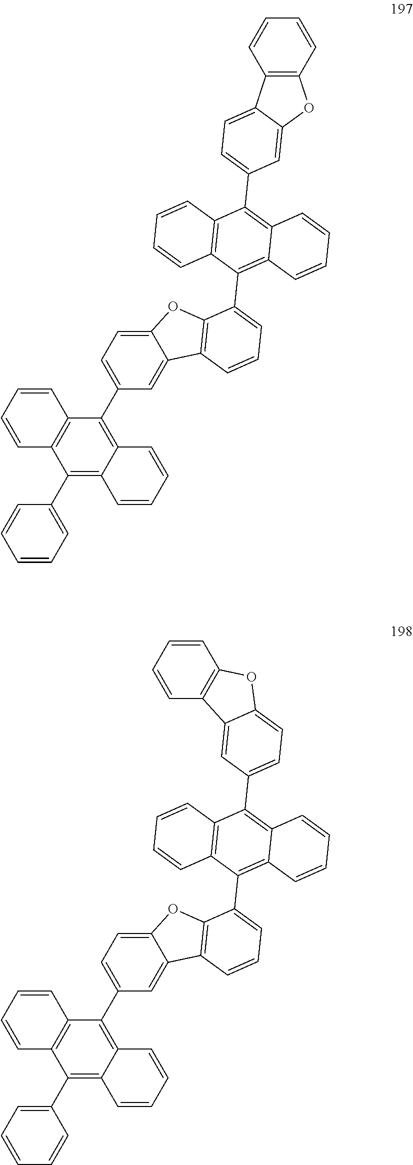 [SMILES: c1ccc(-c2c3ccccc3c(-c3ccc4oc5c(-c6c7ccccc7c(-c7ccc8c(c7)oc7ccccc78)c7ccccc67)cccc5c4c3)c3ccccc23)cc1 ; c1ccc(-c2c3ccccc3c(-c3ccc4oc5c(-c6c7ccccc7c(-c7ccc8oc9ccccc9c8c7)c7ccccc67)cccc5c4c3)c3ccccc23)cc1]

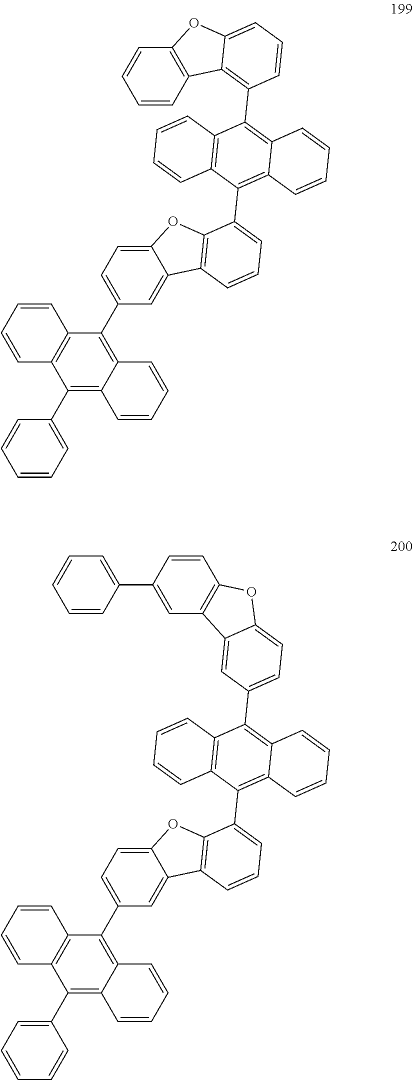 [SMILES: c1ccc(-c2c3ccccc3c(-c3ccc4oc5c(-c6c7ccccc7c(-c7cccc8oc9ccccc9c78)c7ccccc67)cccc5c4c3)c3ccccc23)cc1 ; c1ccc(-c2ccc3oc4ccc(-c5c6ccccc6c(-c6cccc7c6oc6ccc(-c8c9ccccc9c(-c9ccccc9)c9ccccc89)cc67)c6ccccc56)cc4c3c2)cc1]

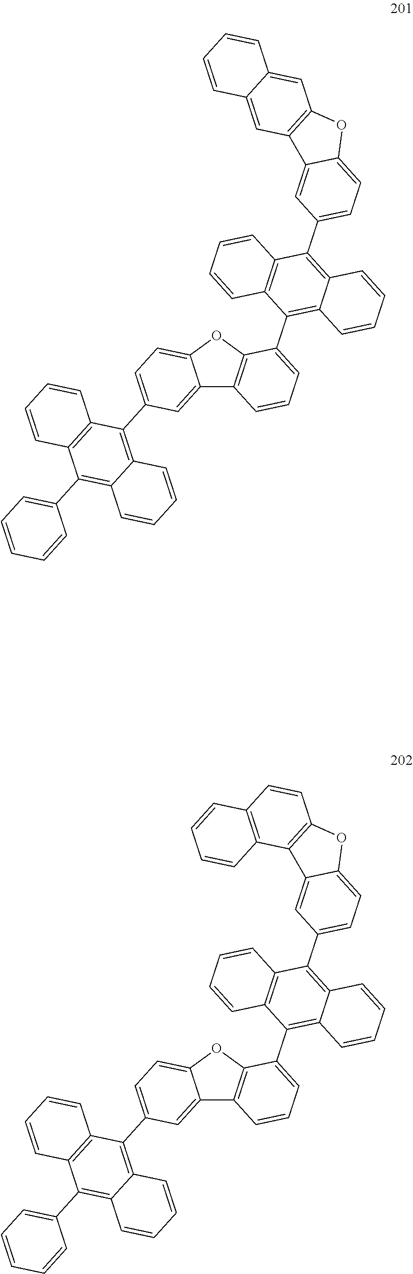 [SMILES: c1ccc(-c2c3ccccc3c(-c3ccc4oc5c(-c6c7ccccc7c(-c7ccc8oc9cc%10ccccc%10cc9c8c7)c7ccccc67)cccc5c4c3)c3ccccc23)cc1 ; c1ccc(-c2c3ccccc3c(-c3ccc4oc5c(-c6c7ccccc7c(-c7ccc8oc9ccc%10ccccc%10c9c8c7)c7ccccc67)cccc5c4c3)c3ccccc23)cc1]

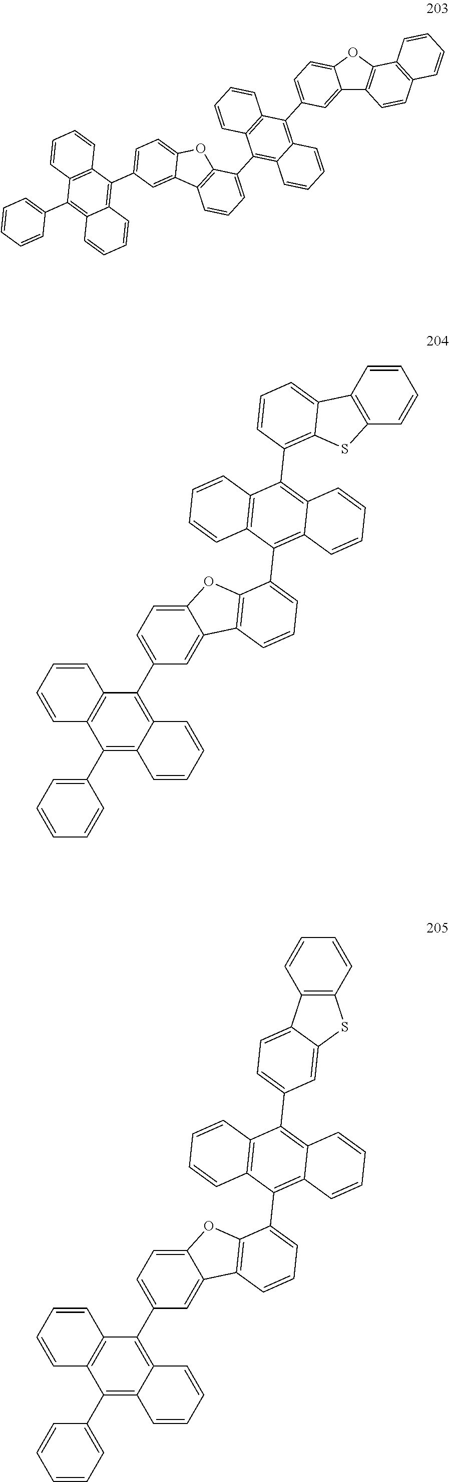 [SMILES: c1ccc(-c2c3ccccc3c(-c3ccc4oc5c(-c6c7ccccc7c(-c7ccc8oc9c%10ccccc%10ccc9c8c7)c7ccccc67)cccc5c4c3)c3ccccc23)cc1 ; c1ccc(-c2c3ccccc3c(-c3ccc4oc5c(-c6c7ccccc7c(-c7cccc8c7sc7ccccc78)c7ccccc67)cccc5c4c3)c3ccccc23)cc1 ; c1ccc(-c2c3ccccc3c(-c3ccc4oc5c(-c6c7ccccc7c(-c7ccc8c(c7)sc7ccccc78)c7ccccc67)cccc5c4c3)c3ccccc23)cc1]

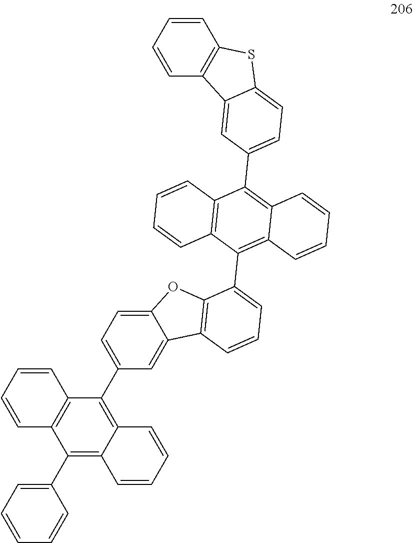 [SMILES: c1ccc(-c2c3ccccc3c(-c3ccc4oc5c(-c6c7ccccc7c(-c7ccc8sc9ccccc9c8c7)c7ccccc67)cccc5c4c3)c3ccccc23)cc1]

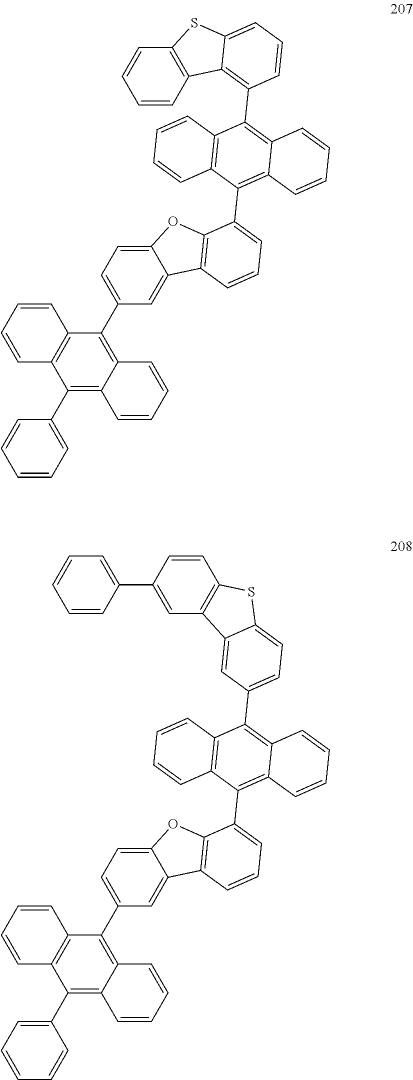 [SMILES: c1ccc(-c2c3ccccc3c(-c3ccc4oc5c(-c6c7ccccc7c(-c7cccc8sc9ccccc9c78)c7ccccc67)cccc5c4c3)c3ccccc23)cc1 ; c1ccc(-c2ccc3sc4ccc(-c5c6ccccc6c(-c6cccc7c6oc6ccc(-c8c9ccccc9c(-c9ccccc9)c9ccccc89)cc67)c6ccccc56)cc4c3c2)cc1]

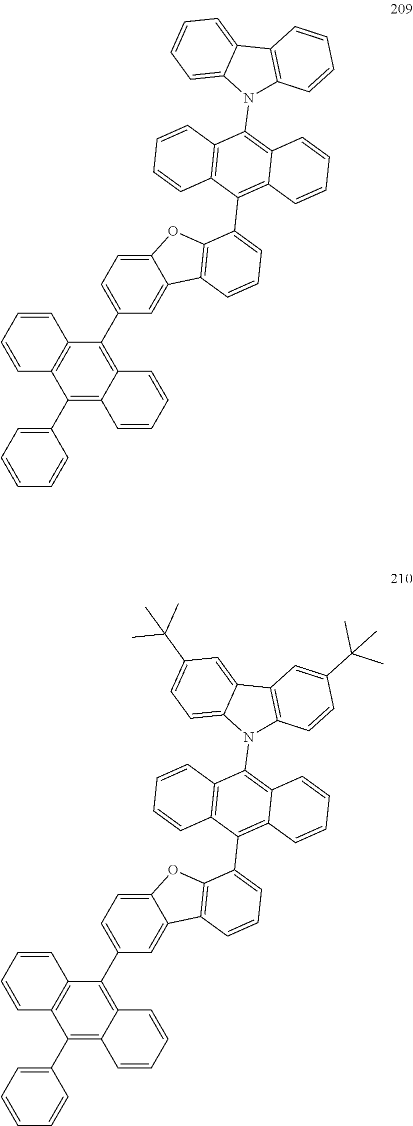 [SMILES: c1ccc(-c2c3ccccc3c(-c3ccc4oc5c(-c6c7ccccc7c(-n7c8ccccc8c8ccccc87)c7ccccc67)cccc5c4c3)c3ccccc23)cc1 ; CC(C)(C)c1ccc2c(c1)c1cc(C(C)(C)C)ccc1n2-c1c2ccccc2c(-c2cccc3c2oc2ccc(-c4c5ccccc5c(-c5ccccc5)c5ccccc45)cc23)c2ccccc12]

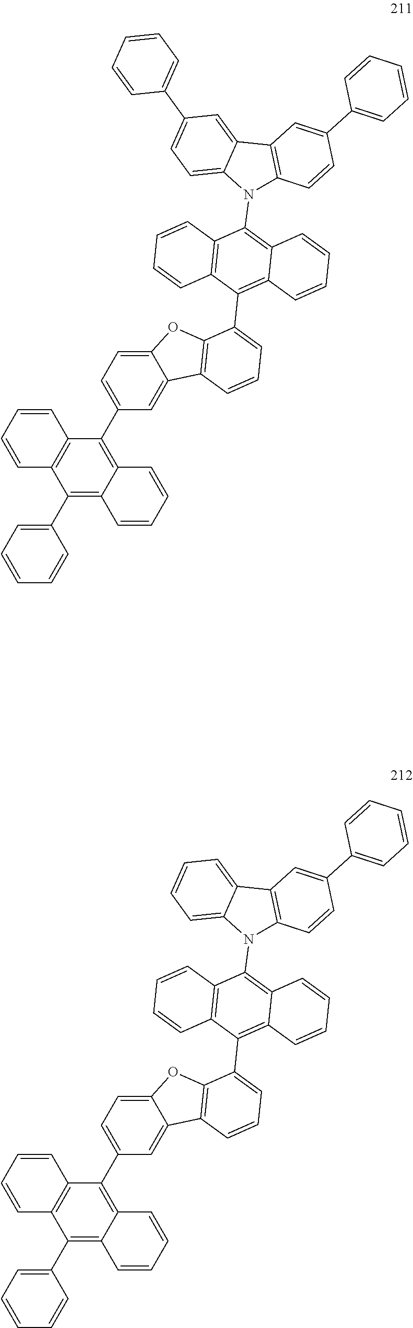 [SMILES: c1ccc(-c2ccc3c(c2)c2cc(-c4ccccc4)ccc2n3-c2c3ccccc3c(-c3cccc4c3oc3ccc(-c5c6ccccc6c(-c6ccccc6)c6ccccc56)cc34)c3ccccc23)cc1 ; c1ccc(-c2ccc3c(c2)c2ccccc2n3-c2c3ccccc3c(-c3cccc4c3oc3ccc(-c5c6ccccc6c(-c6ccccc6)c6ccccc56)cc34)c3ccccc23)cc1]

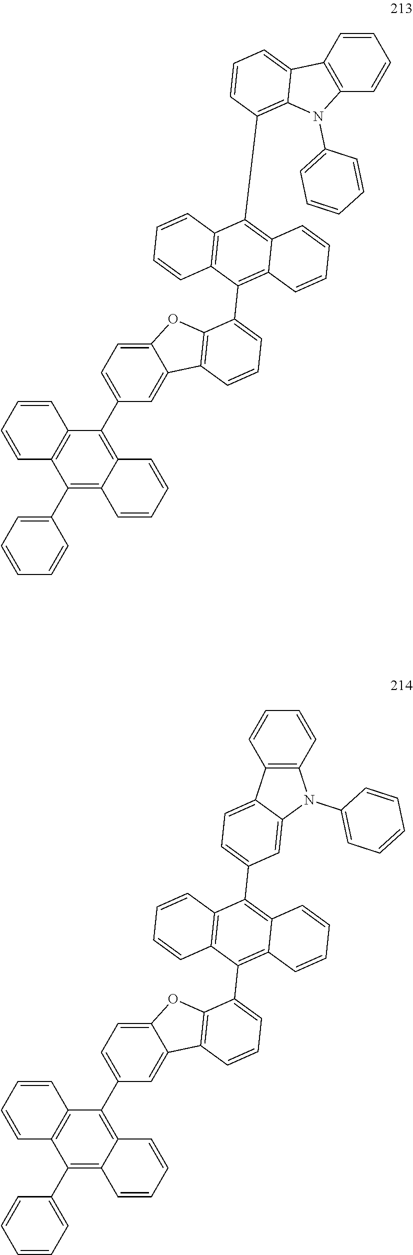 [SMILES: c1ccc(-c2c3ccccc3c(-c3ccc4oc5c(-c6c7ccccc7c(-c7cccc8c9ccccc9n(-c9ccccc9)c78)c7ccccc67)cccc5c4c3)c3ccccc23)cc1 ; c1ccc(-c2c3ccccc3c(-c3ccc4oc5c(-c6c7ccccc7c(-c7ccc8c9ccccc9n(-c9ccccc9)c8c7)c7ccccc67)cccc5c4c3)c3ccccc23)cc1]

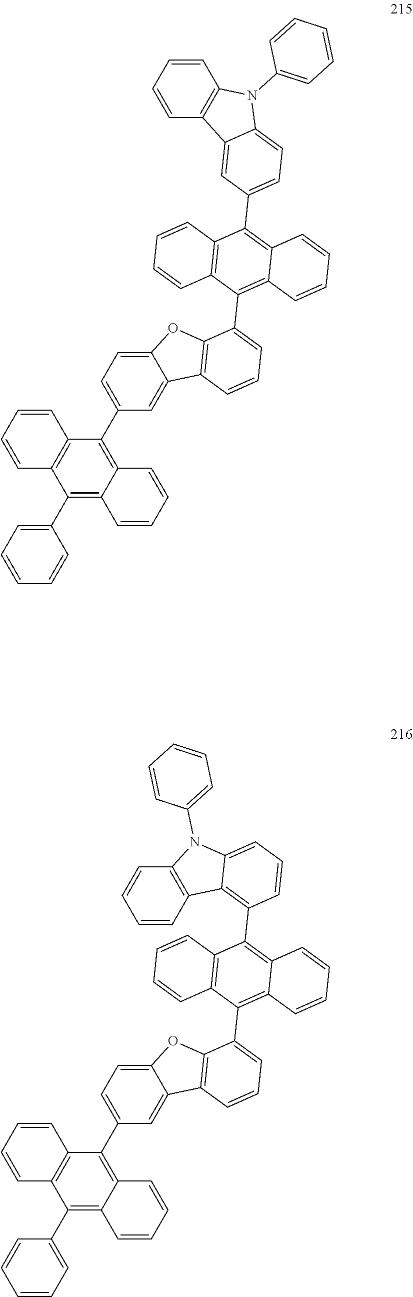 [SMILES: c1ccc(-c2c3ccccc3c(-c3ccc4oc5c(-c6c7ccccc7c(-c7ccc8c(c7)c7ccccc7n8-c7ccccc7)c7ccccc67)cccc5c4c3)c3ccccc23)cc1 ; c1ccc(-c2c3ccccc3c(-c3ccc4oc5c(-c6c7ccccc7c(-c7cccc8c7c7ccccc7n8-c7ccccc7)c7ccccc67)cccc5c4c3)c3ccccc23)cc1]

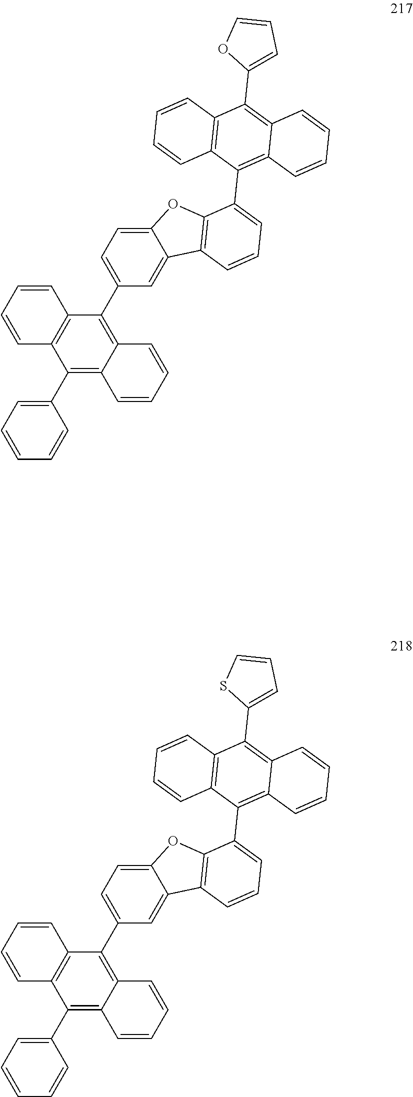 [SMILES: c1ccc(-c2c3ccccc3c(-c3ccc4oc5c(-c6c7ccccc7c(-c7ccco7)c7ccccc67)cccc5c4c3)c3ccccc23)cc1 ; c1ccc(-c2c3ccccc3c(-c3ccc4oc5c(-c6c7ccccc7c(-c7cccs7)c7ccccc67)cccc5c4c3)c3ccccc23)cc1]

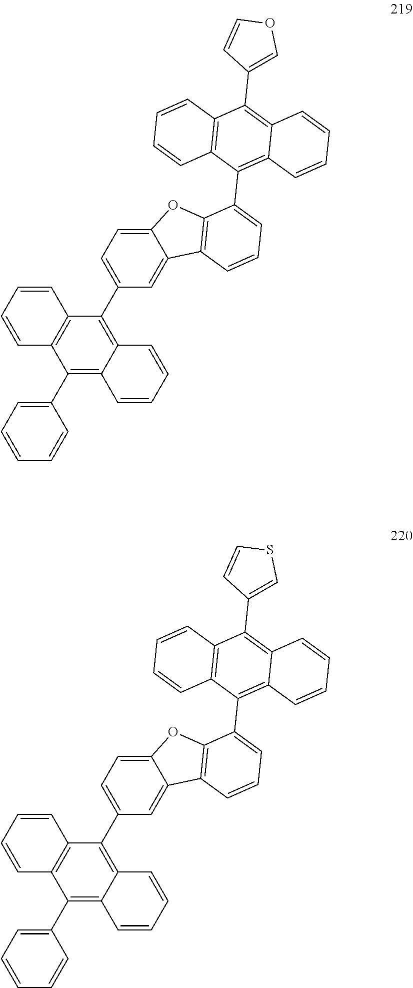 [SMILES: c1ccc(-c2c3ccccc3c(-c3ccc4oc5c(-c6c7ccccc7c(-c7ccoc7)c7ccccc67)cccc5c4c3)c3ccccc23)cc1 ; c1ccc(-c2c3ccccc3c(-c3ccc4oc5c(-c6c7ccccc7c(-c7ccsc7)c7ccccc67)cccc5c4c3)c3ccccc23)cc1]

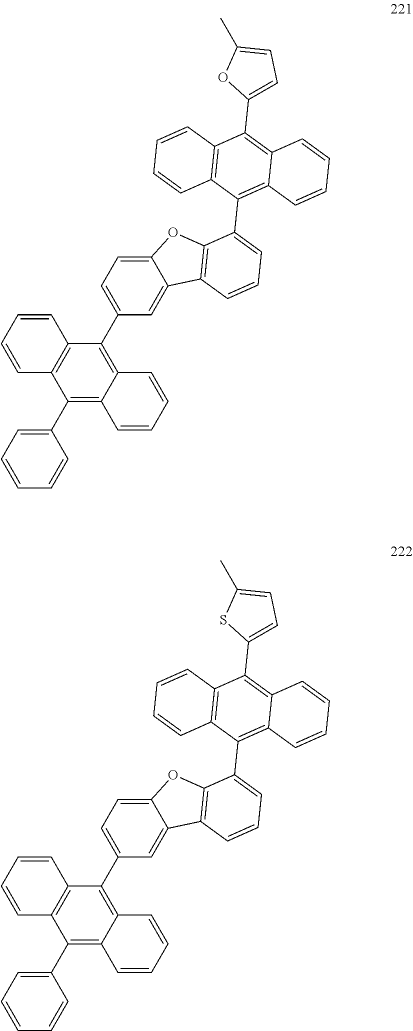 [SMILES: Cc1ccc(-c2c3ccccc3c(-c3cccc4c3oc3ccc(-c5c6ccccc6c(-c6ccccc6)c6ccccc56)cc34)c3ccccc23)o1 ; Cc1ccc(-c2c3ccccc3c(-c3cccc4c3oc3ccc(-c5c6ccccc6c(-c6ccccc6)c6ccccc56)cc34)c3ccccc23)s1]

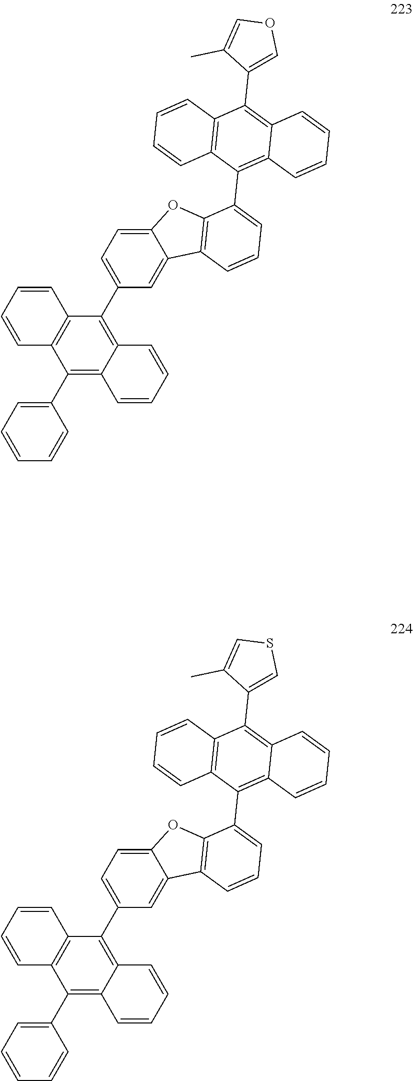 [SMILES: Cc1cocc1-c1c2ccccc2c(-c2cccc3c2oc2ccc(-c4c5ccccc5c(-c5ccccc5)c5ccccc45)cc23)c2ccccc12 ; Cc1cscc1-c1c2ccccc2c(-c2cccc3c2oc2ccc(-c4c5ccccc5c(-c5ccccc5)c5ccccc45)cc23)c2ccccc12]

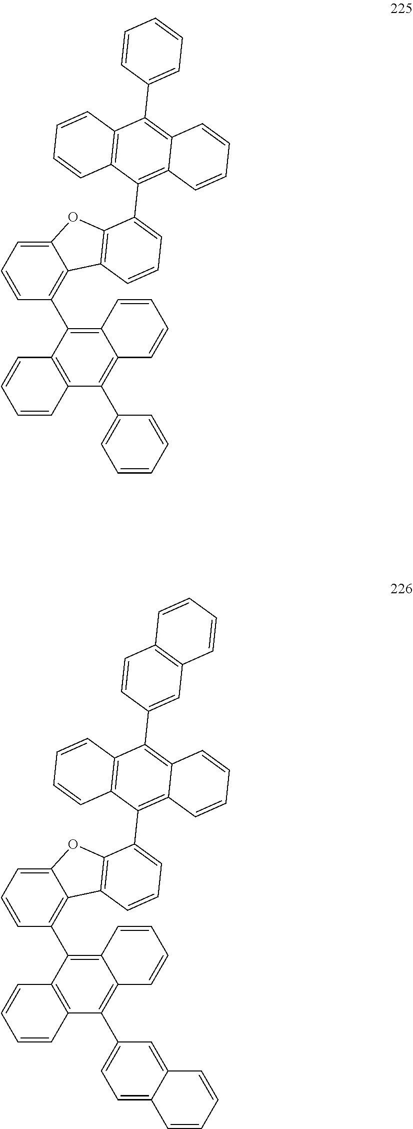 [SMILES: c1ccc(-c2c3ccccc3c(-c3cccc4c3oc3cccc(-c5c6ccccc6c(-c6ccccc6)c6ccccc56)c34)c3ccccc23)cc1 ; c1ccc2cc(-c3c4ccccc4c(-c4cccc5c4oc4cccc(-c6c7ccccc7c(-c7ccc8ccccc8c7)c7ccccc67)c45)c4ccccc34)ccc2c1]

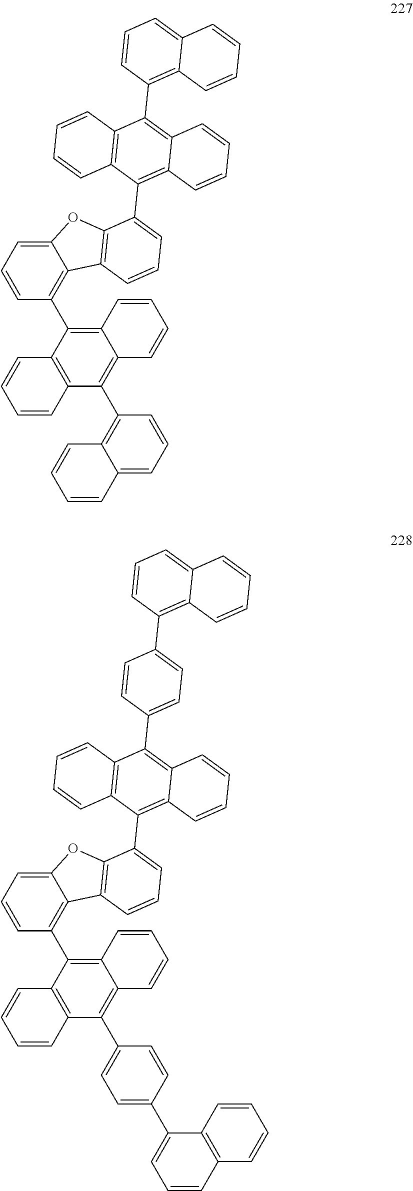 [SMILES: c1ccc2c(-c3c4ccccc4c(-c4cccc5c4oc4cccc(-c6c7ccccc7c(-c7cccc8ccccc78)c7ccccc67)c45)c4ccccc34)cccc2c1 ; c1ccc2c(-c3ccc(-c4c5ccccc5c(-c5cccc6c5oc5cccc(-c7c8ccccc8c(-c8ccc(-c9cccc%10ccccc9%10)cc8)c8ccccc78)c56)c5ccccc45)cc3)cccc2c1]

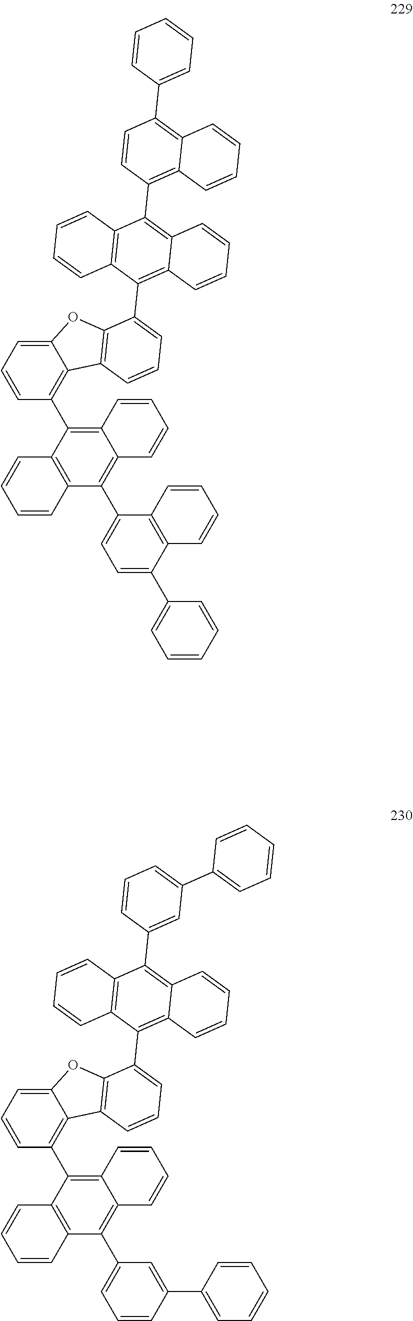 [SMILES: c1ccc(-c2ccc(-c3c4ccccc4c(-c4cccc5c4oc4cccc(-c6c7ccccc7c(-c7ccc(-c8ccccc8)c8ccccc78)c7ccccc67)c45)c4ccccc34)c3ccccc23)cc1 ; c1ccc(-c2cccc(-c3c4ccccc4c(-c4cccc5c4oc4cccc(-c6c7ccccc7c(-c7cccc(-c8ccccc8)c7)c7ccccc67)c45)c4ccccc34)c2)cc1]

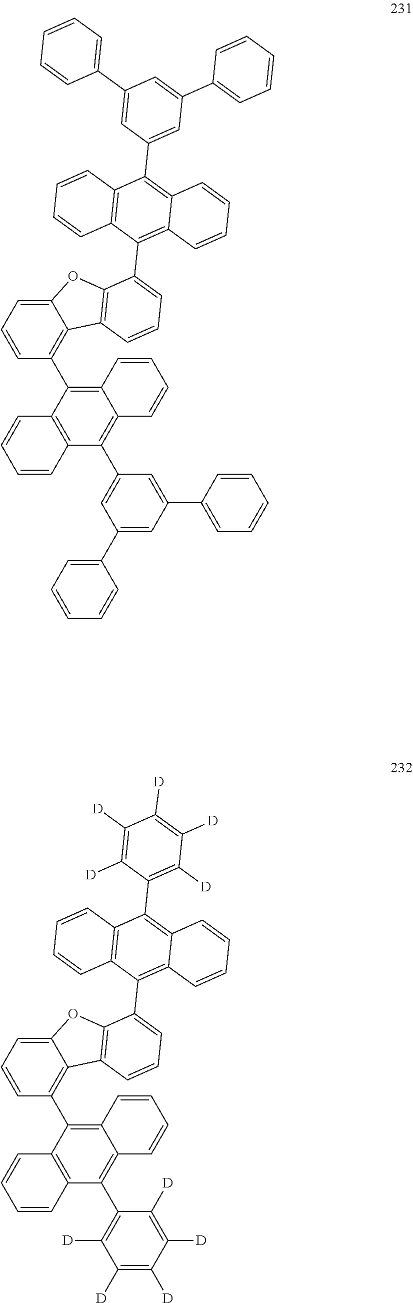 [SMILES: c1ccc(-c2cc(-c3ccccc3)cc(-c3c4ccccc4c(-c4cccc5c4oc4cccc(-c6c7ccccc7c(-c7cc(-c8ccccc8)cc(-c8ccccc8)c7)c7ccccc67)c45)c4ccccc34)c2)cc1 ; [2H]c1c([2H])c([2H])c(-c2c3ccccc3c(-c3cccc4c3oc3cccc(-c5c6ccccc6c(-c6c([2H])c([2H])c([2H])c([2H])c6[2H])c6ccccc56)c34)c3ccccc23)c([2H])c1[2H]]

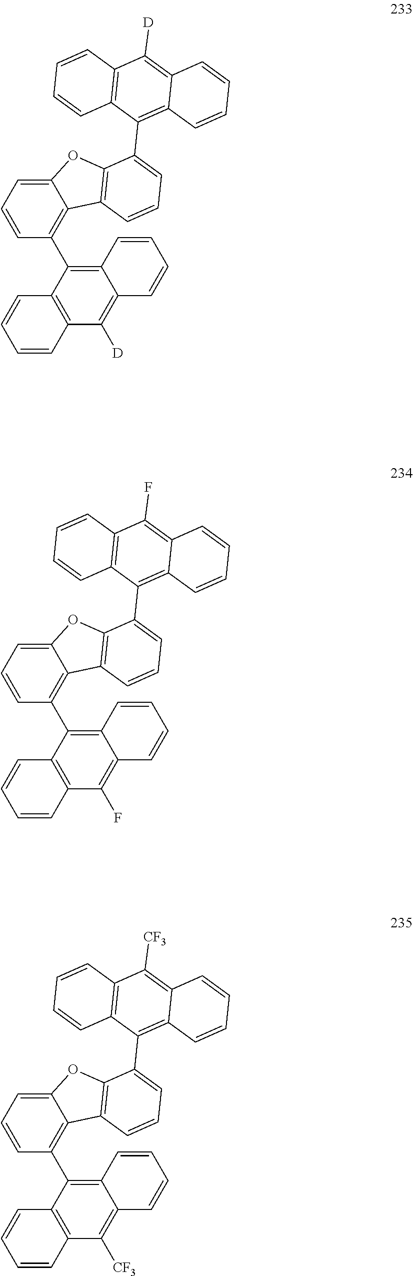 [SMILES: [2H]c1c2ccccc2c(-c2cccc3c2oc2cccc(-c4c5ccccc5c([2H])c5ccccc45)c23)c2ccccc12 ; Fc1c2ccccc2c(-c2cccc3c2oc2cccc(-c4c5ccccc5c(F)c5ccccc45)c23)c2ccccc12 ; FC(F)(F)c1c2ccccc2c(-c2cccc3c2oc2cccc(-c4c5ccccc5c(C(F)(F)F)c5ccccc45)c23)c2ccccc12]

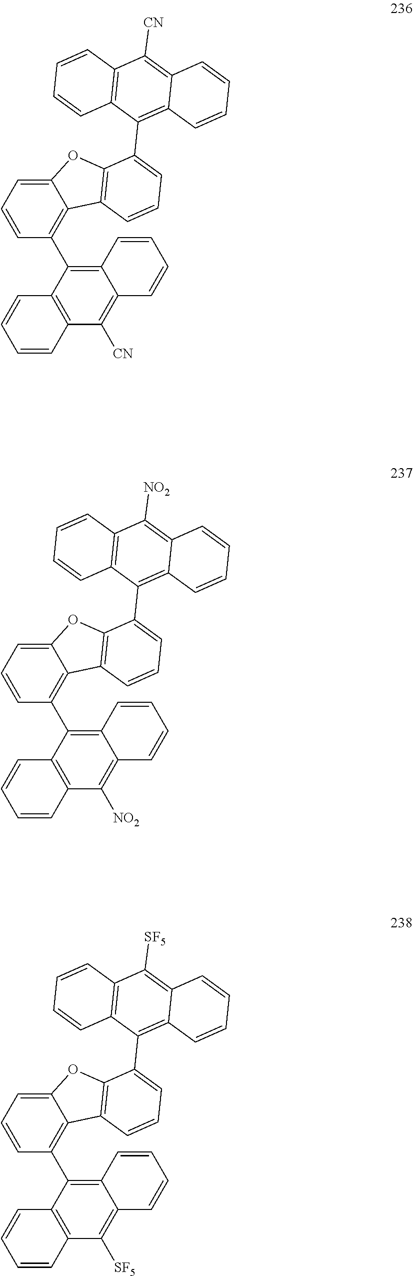

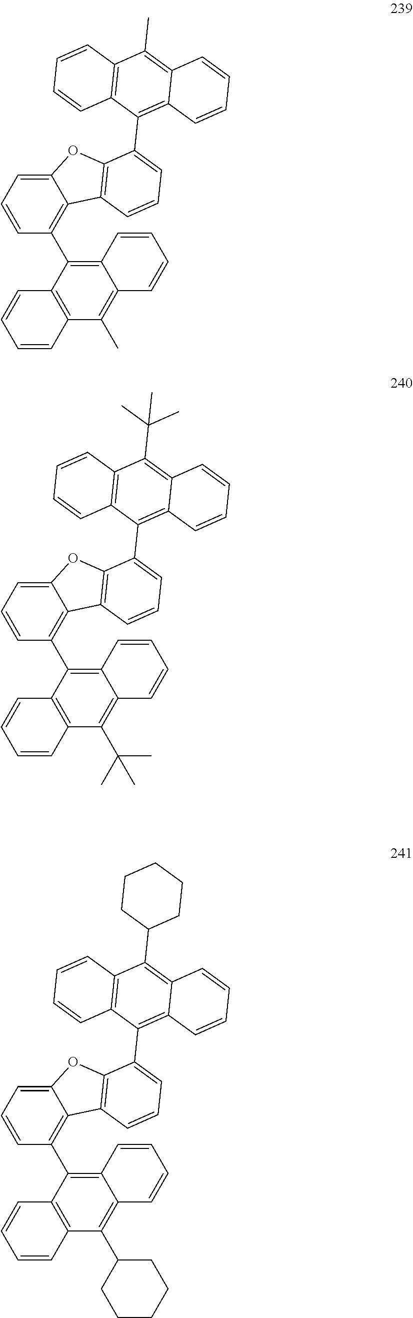 [SMILES: Cc1c2ccccc2c(-c2cccc3c2oc2cccc(-c4c5ccccc5c(C)c5ccccc45)c23)c2ccccc12 ; CC(C)(C)c1c2ccccc2c(-c2cccc3c2oc2cccc(-c4c5ccccc5c(C(C)(C)C)c5ccccc45)c23)c2ccccc12 ; c1cc(-c2c3ccccc3c(C3CCCCC3)c3ccccc23)c2c(c1)oc1c(-c3c4ccccc4c(C4CCCCC4)c4ccccc34)cccc12]

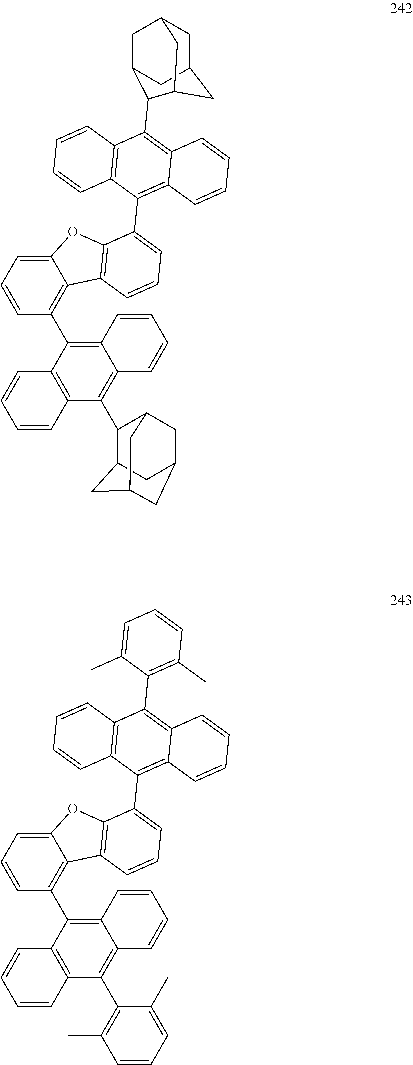 [SMILES: c1cc(-c2c3ccccc3c(C3C4CC5CC(C4)CC3C5)c3ccccc23)c2c(c1)oc1c(-c3c4ccccc4c(C4C5CC6CC(C5)CC4C6)c4ccccc34)cccc12 ; Cc1cccc(C)c1-c1c2ccccc2c(-c2cccc3c2oc2cccc(-c4c5ccccc5c(-c5c(C)cccc5C)c5ccccc45)c23)c2ccccc12]

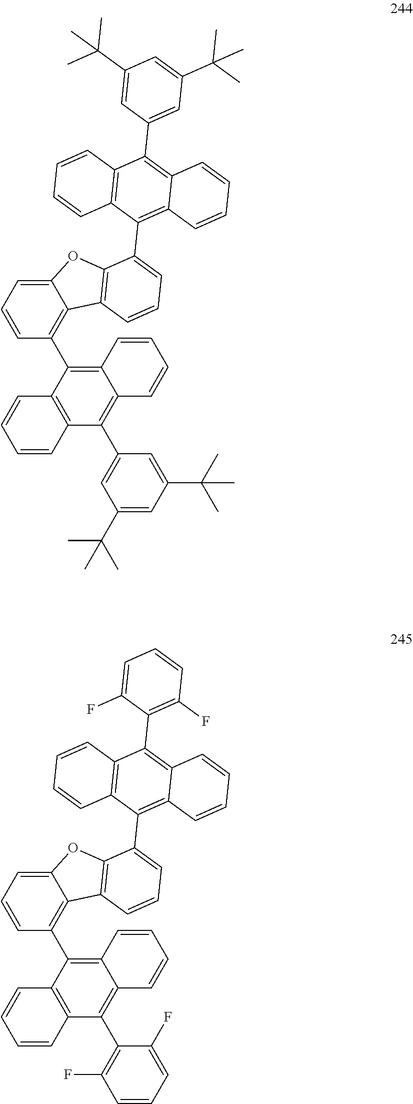 [SMILES: CC(C)(C)c1cc(-c2c3ccccc3c(-c3cccc4c3oc3cccc(-c5c6ccccc6c(-c6cc(C(C)(C)C)cc(C(C)(C)C)c6)c6ccccc56)c34)c3ccccc23)cc(C(C)(C)C)c1 ; Fc1cccc(F)c1-c1c2ccccc2c(-c2cccc3c2oc2cccc(-c4c5ccccc5c(-c5c(F)cccc5F)c5ccccc45)c23)c2ccccc12]

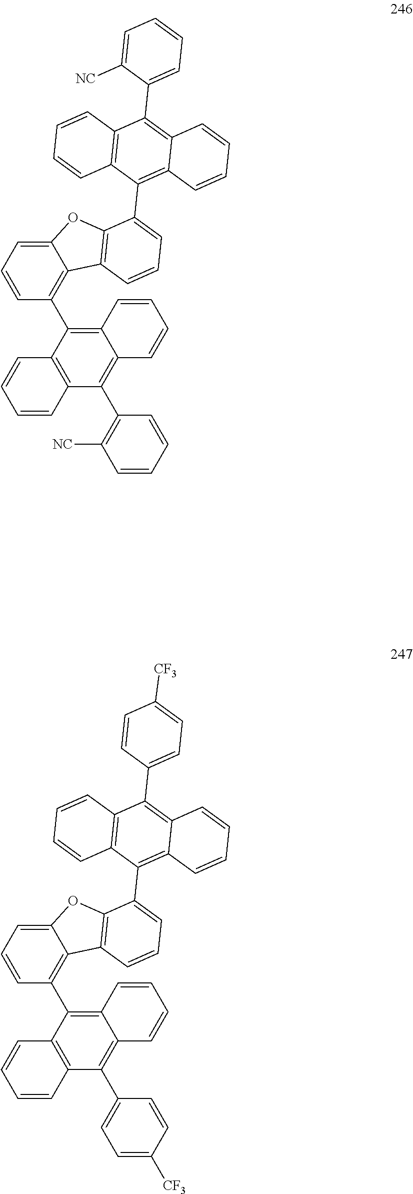 [SMILES: N#Cc1ccccc1-c1c2ccccc2c(-c2cccc3c2oc2cccc(-c4c5ccccc5c(-c5ccccc5C#N)c5ccccc45)c23)c2ccccc12 ; FC(F)(F)c1ccc(-c2c3ccccc3c(-c3cccc4c3oc3cccc(-c5c6ccccc6c(-c6ccc(C(F)(F)F)cc6)c6ccccc56)c34)c3ccccc23)cc1]

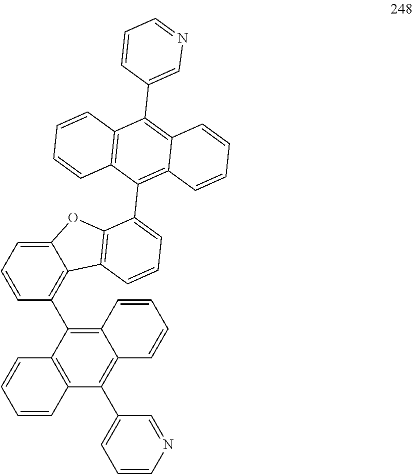 [SMILES: c1cncc(-c2c3ccccc3c(-c3cccc4c3oc3cccc(-c5c6ccccc6c(-c6cccnc6)c6ccccc56)c34)c3ccccc23)c1]

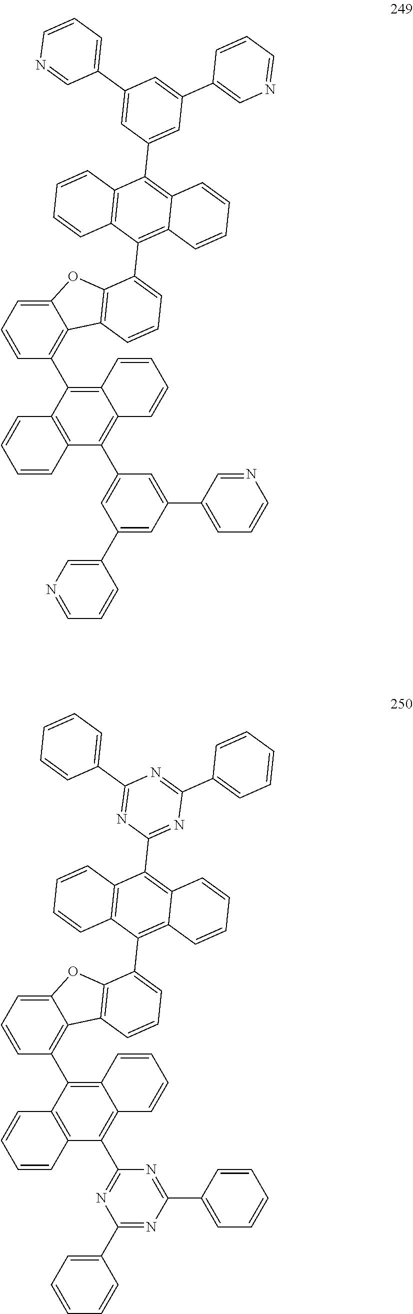 [SMILES: c1cncc(-c2cc(-c3cccnc3)cc(-c3c4ccccc4c(-c4cccc5c4oc4cccc(-c6c7ccccc7c(-c7cc(-c8cccnc8)cc(-c8cccnc8)c7)c7ccccc67)c45)c4ccccc34)c2)c1 ; c1ccc(-c2nc(-c3ccccc3)nc(-c3c4ccccc4c(-c4cccc5c4oc4cccc(-c6c7ccccc7c(-c7nc(-c8ccccc8)nc(-c8ccccc8)n7)c7ccccc67)c45)c4ccccc34)n2)cc1]

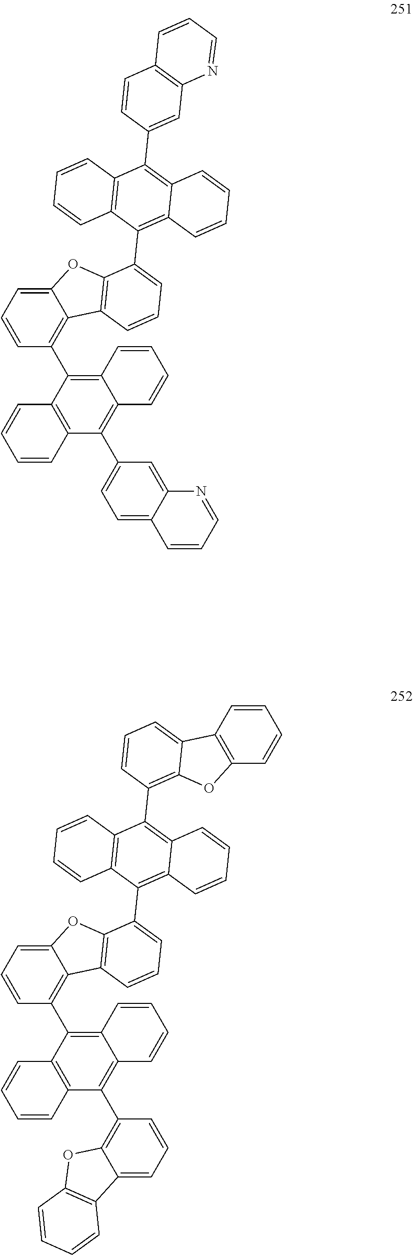 [SMILES: c1cnc2cc(-c3c4ccccc4c(-c4cccc5c4oc4cccc(-c6c7ccccc7c(-c7ccc8cccnc8c7)c7ccccc67)c45)c4ccccc34)ccc2c1 ; c1ccc2c(c1)oc1c(-c3c4ccccc4c(-c4cccc5c4oc4cccc(-c6c7ccccc7c(-c7cccc8c7oc7ccccc78)c7ccccc67)c45)c4ccccc34)cccc12]

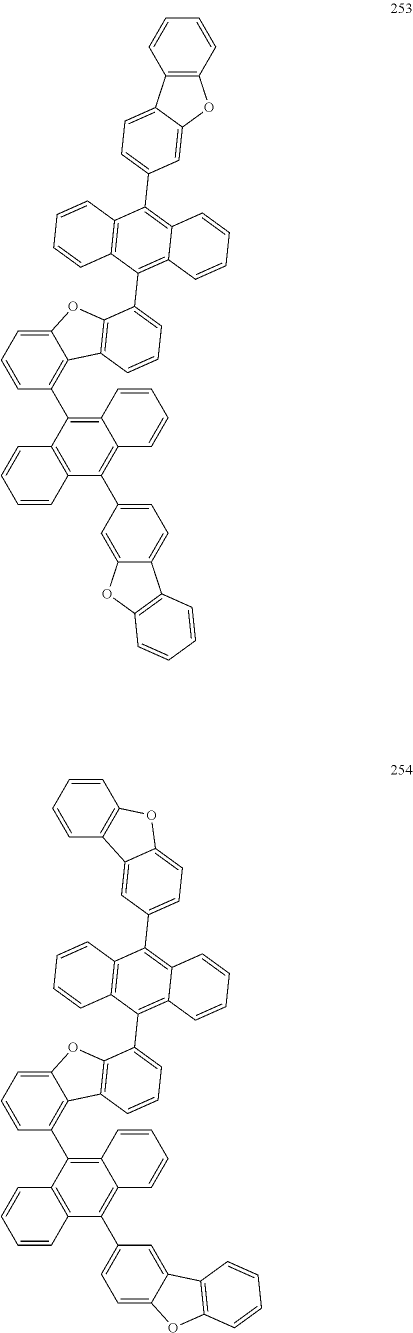 [SMILES: c1ccc2c(c1)oc1cc(-c3c4ccccc4c(-c4cccc5c4oc4cccc(-c6c7ccccc7c(-c7ccc8c(c7)oc7ccccc78)c7ccccc67)c45)c4ccccc34)ccc12 ; c1ccc2c(c1)oc1ccc(-c3c4ccccc4c(-c4cccc5c4oc4cccc(-c6c7ccccc7c(-c7ccc8oc9ccccc9c8c7)c7ccccc67)c45)c4ccccc34)cc12]

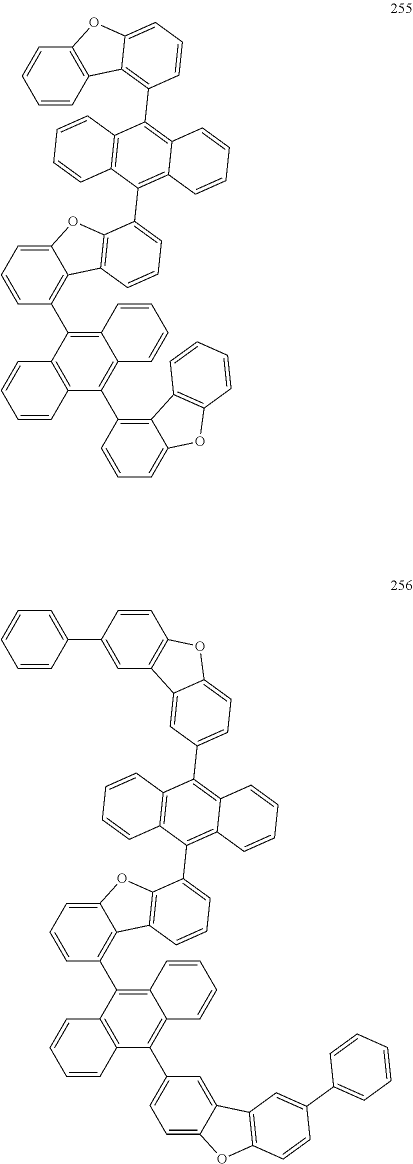 [SMILES: c1ccc2c(c1)oc1cccc(-c3c4ccccc4c(-c4cccc5c4oc4cccc(-c6c7ccccc7c(-c7cccc8oc9ccccc9c78)c7ccccc67)c45)c4ccccc34)c12 ; c1ccc(-c2ccc3oc4ccc(-c5c6ccccc6c(-c6cccc7c6oc6cccc(-c8c9ccccc9c(-c9ccc%10oc%11ccc(-c%12ccccc%12)cc%11c%10c9)c9ccccc89)c67)c6ccccc56)cc4c3c2)cc1]

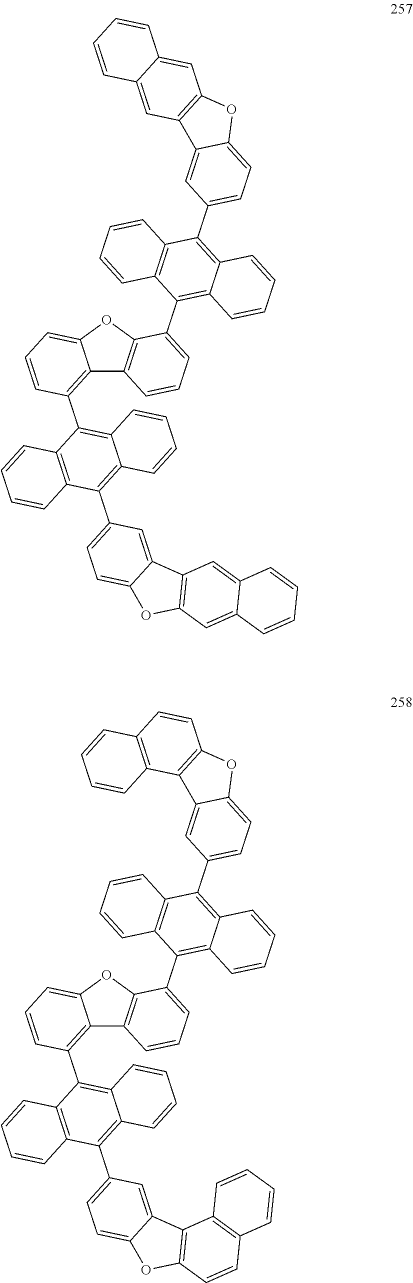 [SMILES: c1ccc2cc3c(cc2c1)oc1ccc(-c2c4ccccc4c(-c4cccc5c4oc4cccc(-c6c7ccccc7c(-c7ccc8oc9cc%10ccccc%10cc9c8c7)c7ccccc67)c45)c4ccccc24)cc13 ; c1ccc2c(c1)ccc1oc3ccc(-c4c5ccccc5c(-c5cccc6c5oc5cccc(-c7c8ccccc8c(-c8ccc9oc%10ccc%11ccccc%11c%10c9c8)c8ccccc78)c56)c5ccccc45)cc3c12]

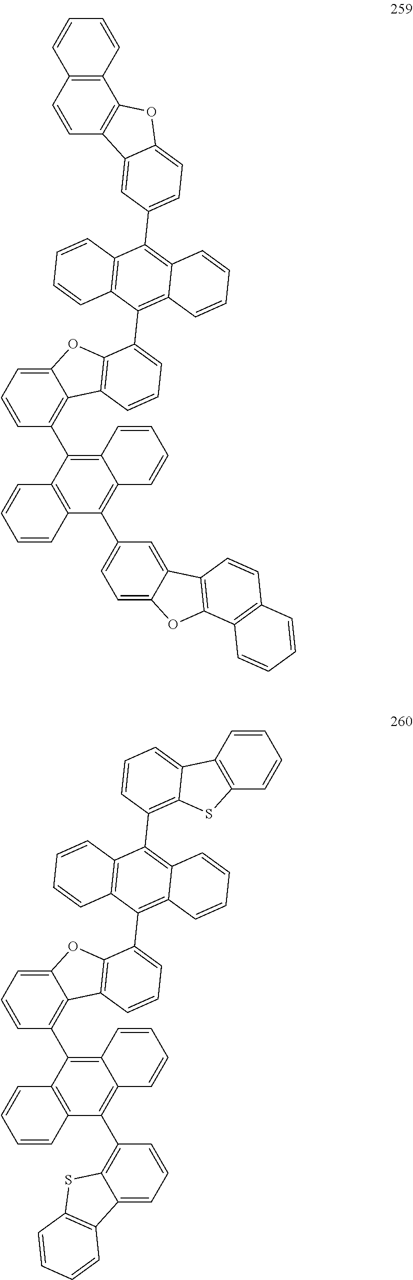 [SMILES: c1ccc2c(c1)ccc1c3cc(-c4c5ccccc5c(-c5cccc6c5oc5cccc(-c7c8ccccc8c(-c8ccc9oc%10c%11ccccc%11ccc%10c9c8)c8ccccc78)c56)c5ccccc45)ccc3oc21 ; c1cc(-c2c3ccccc3c(-c3cccc4c3sc3ccccc34)c3ccccc23)c2c(c1)oc1c(-c3c4ccccc4c(-c4cccc5c4sc4ccccc45)c4ccccc34)cccc12]

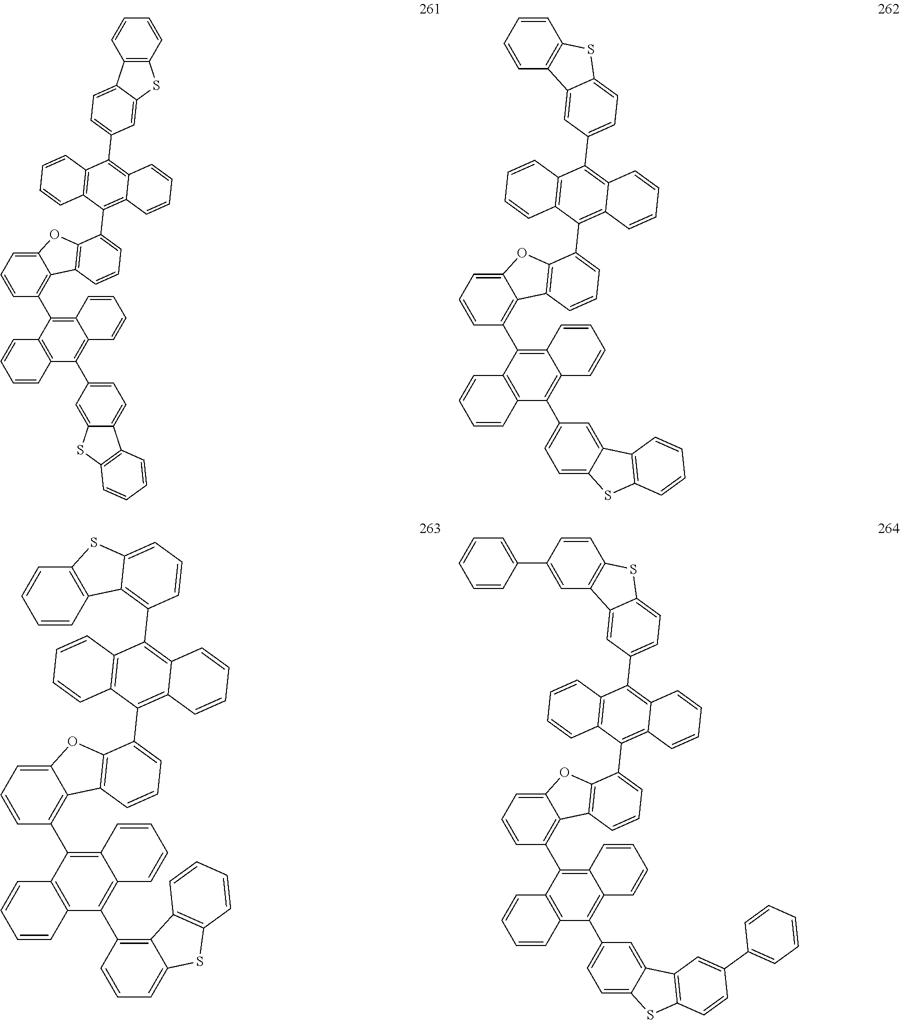 [SMILES: c1cc(-c2c3ccccc3c(-c3ccc4c(c3)sc3ccccc34)c3ccccc23)c2c(c1)oc1c(-c3c4ccccc4c(-c4ccc5c(c4)sc4ccccc45)c4ccccc34)cccc12 ; c1cc(-c2c3ccccc3c(-c3ccc4sc5ccccc5c4c3)c3ccccc23)c2c(c1)oc1c(-c3c4ccccc4c(-c4ccc5sc6ccccc6c5c4)c4ccccc34)cccc12 ; c1cc(-c2c3ccccc3c(-c3cccc4sc5ccccc5c34)c3ccccc23)c2c(c1)oc1c(-c3c4ccccc4c(-c4cccc5sc6ccccc6c45)c4ccccc34)cccc12 ; c1ccc(-c2ccc3sc4ccc(-c5c6ccccc6c(-c6cccc7c6oc6cccc(-c8c9ccccc9c(-c9ccc%10sc%11ccc(-c%12ccccc%12)cc%11c%10c9)c9ccccc89)c67)c6ccccc56)cc4c3c2)cc1]

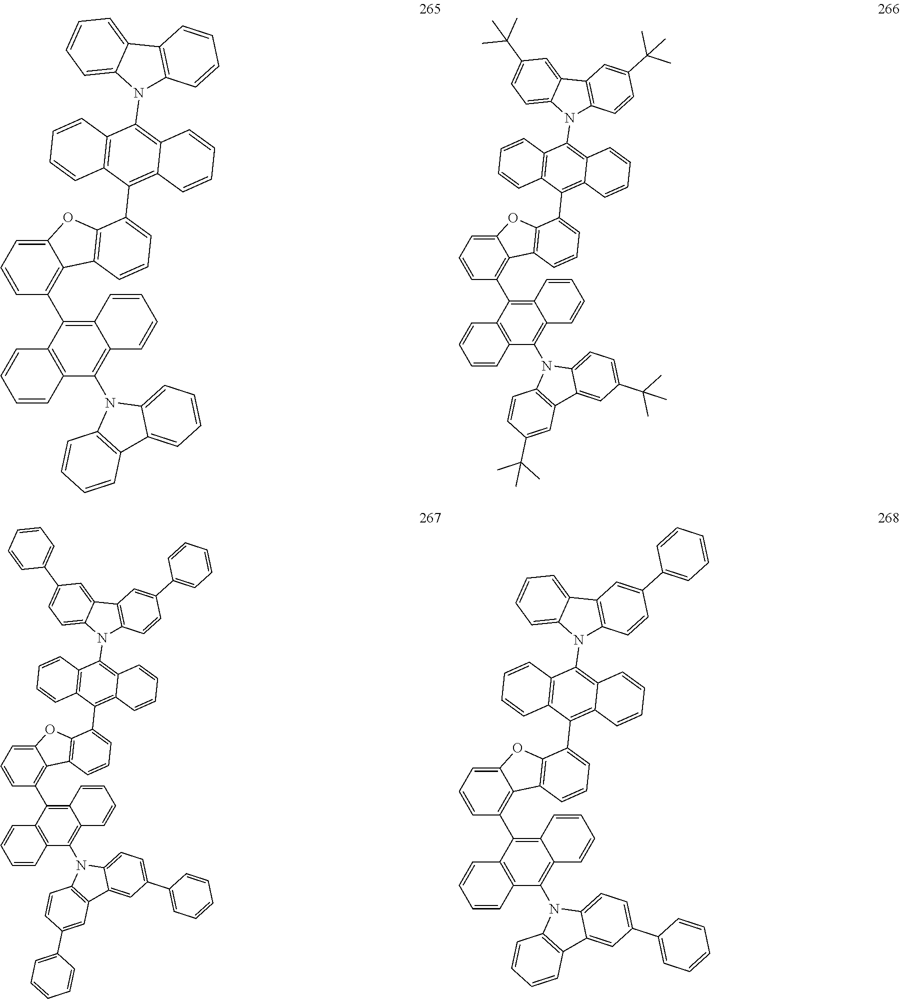

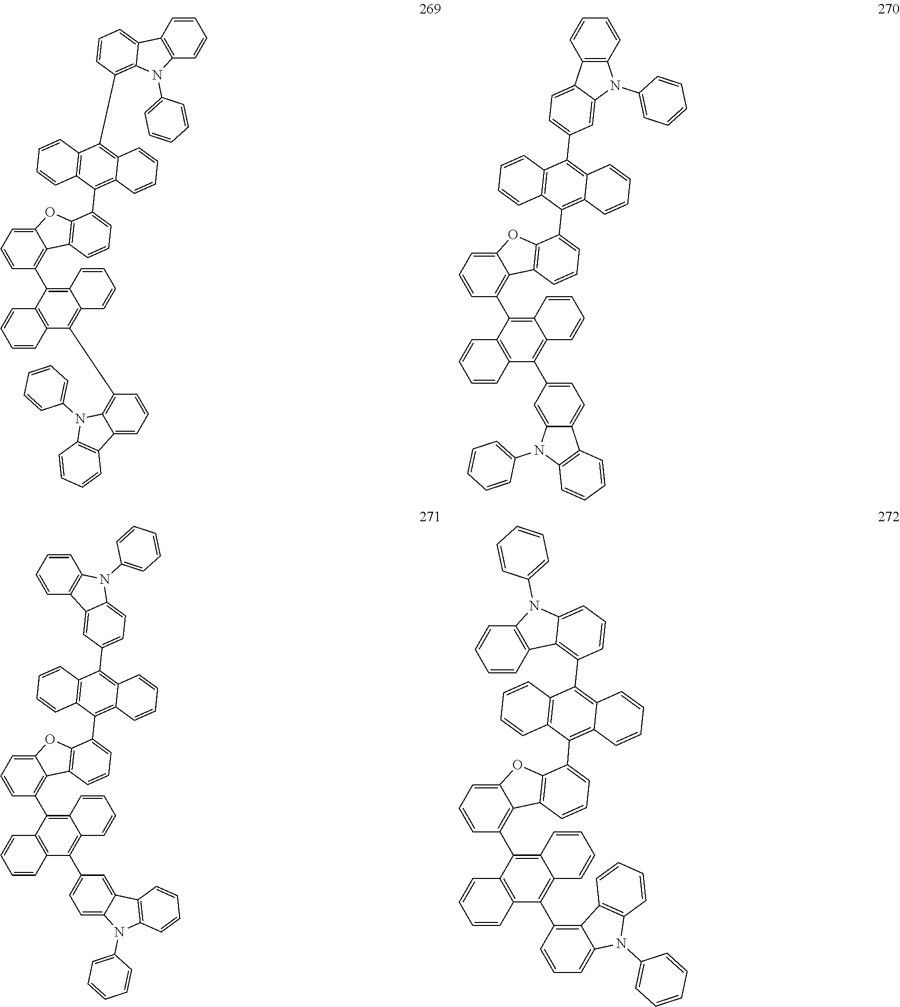

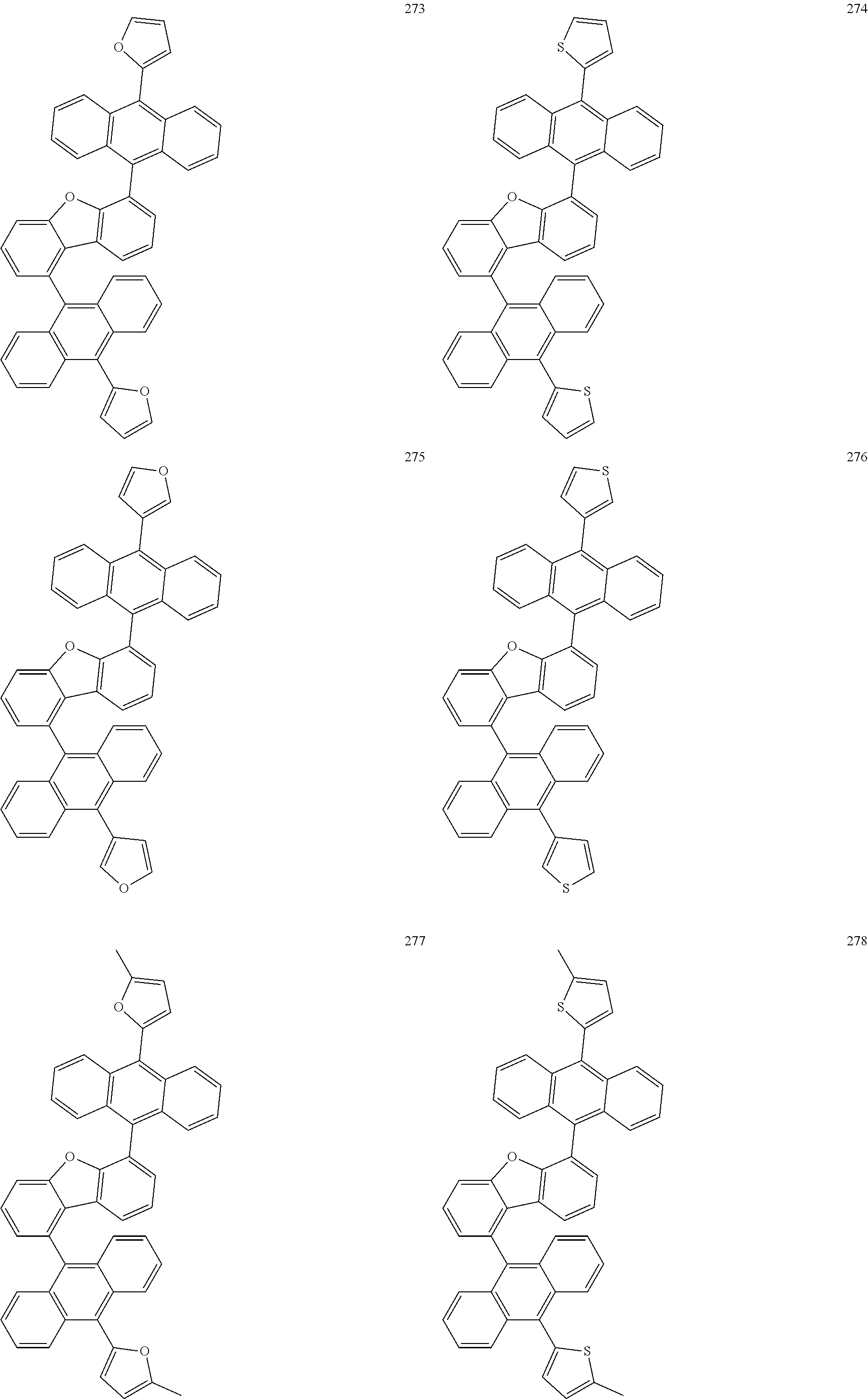

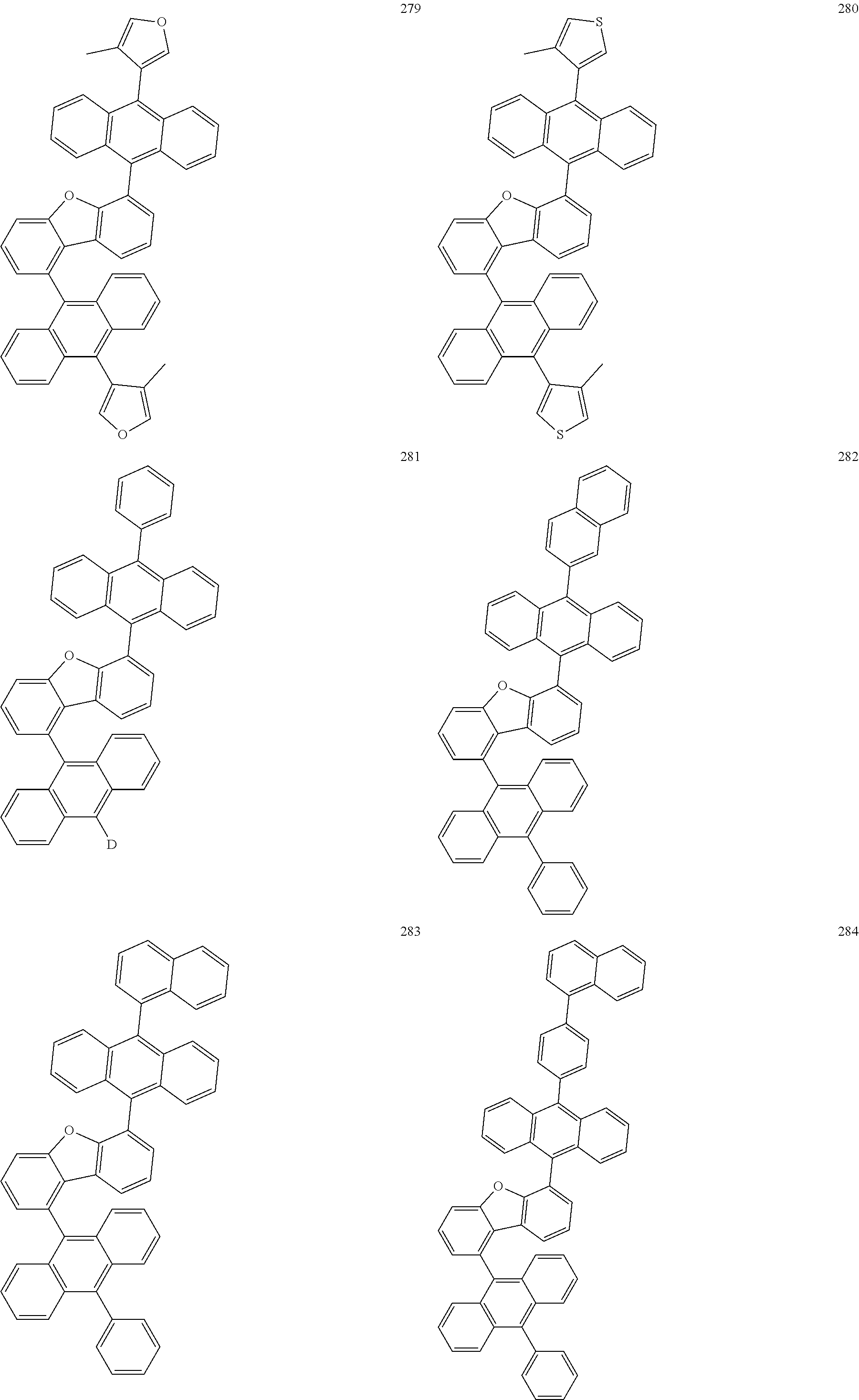

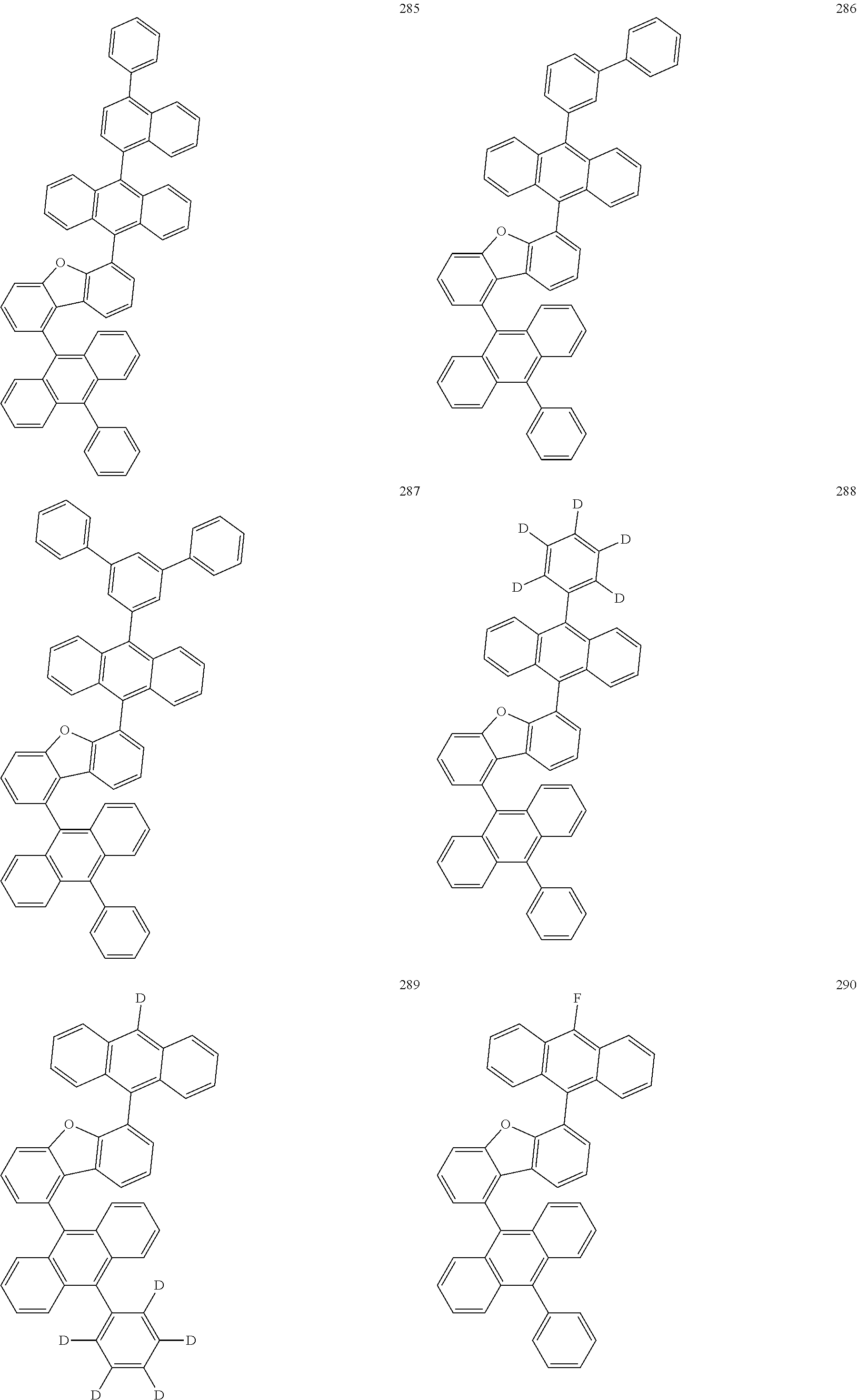

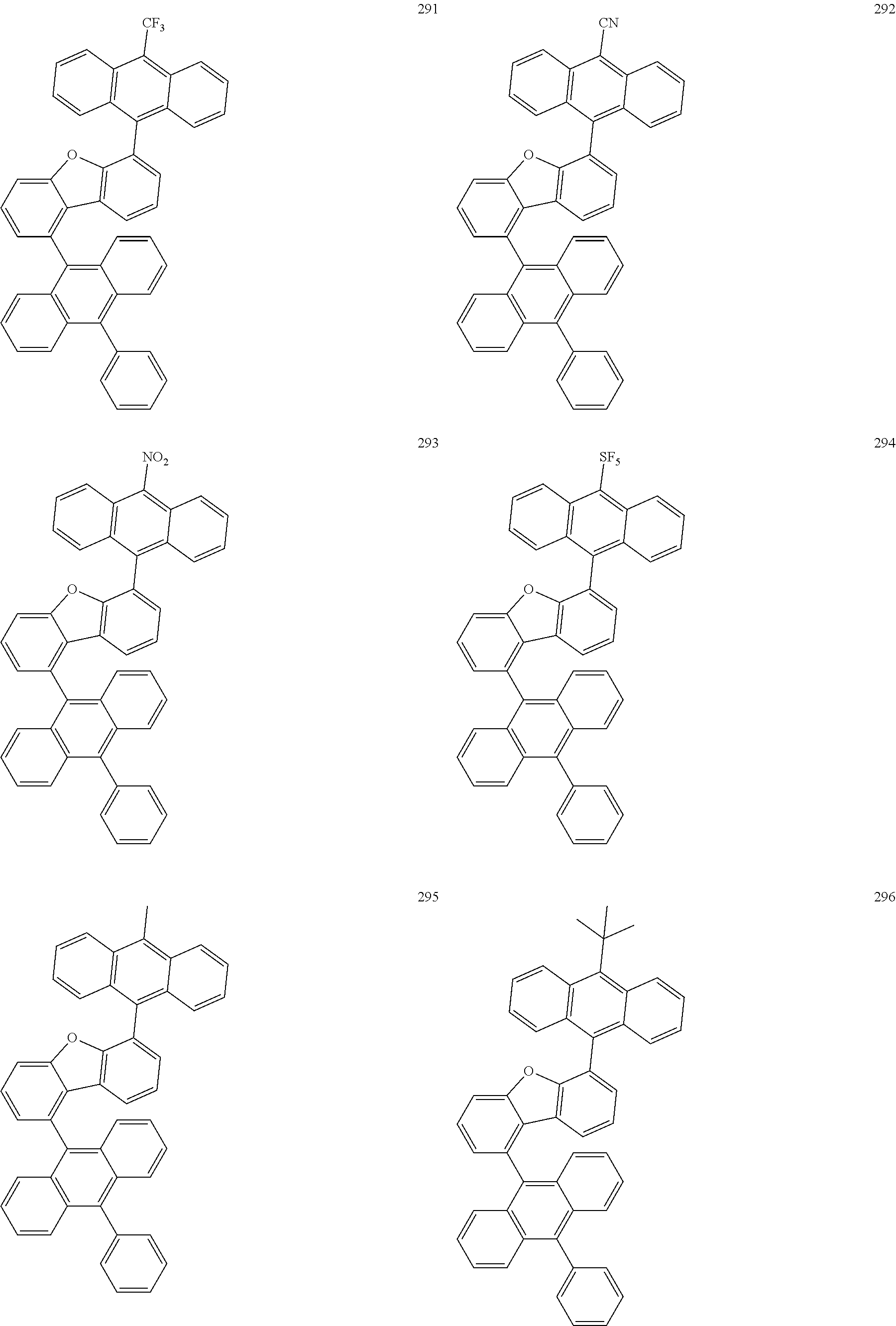

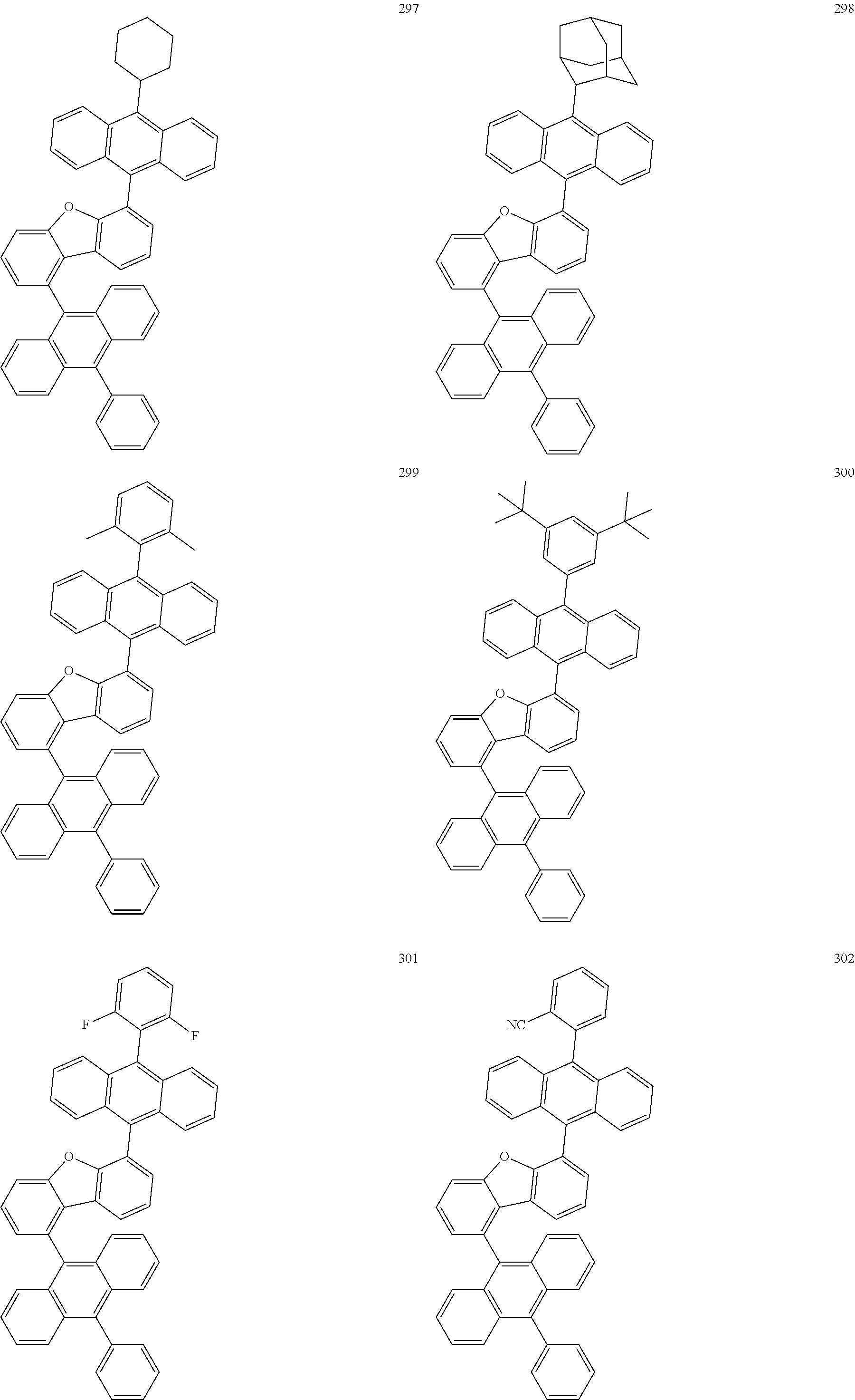

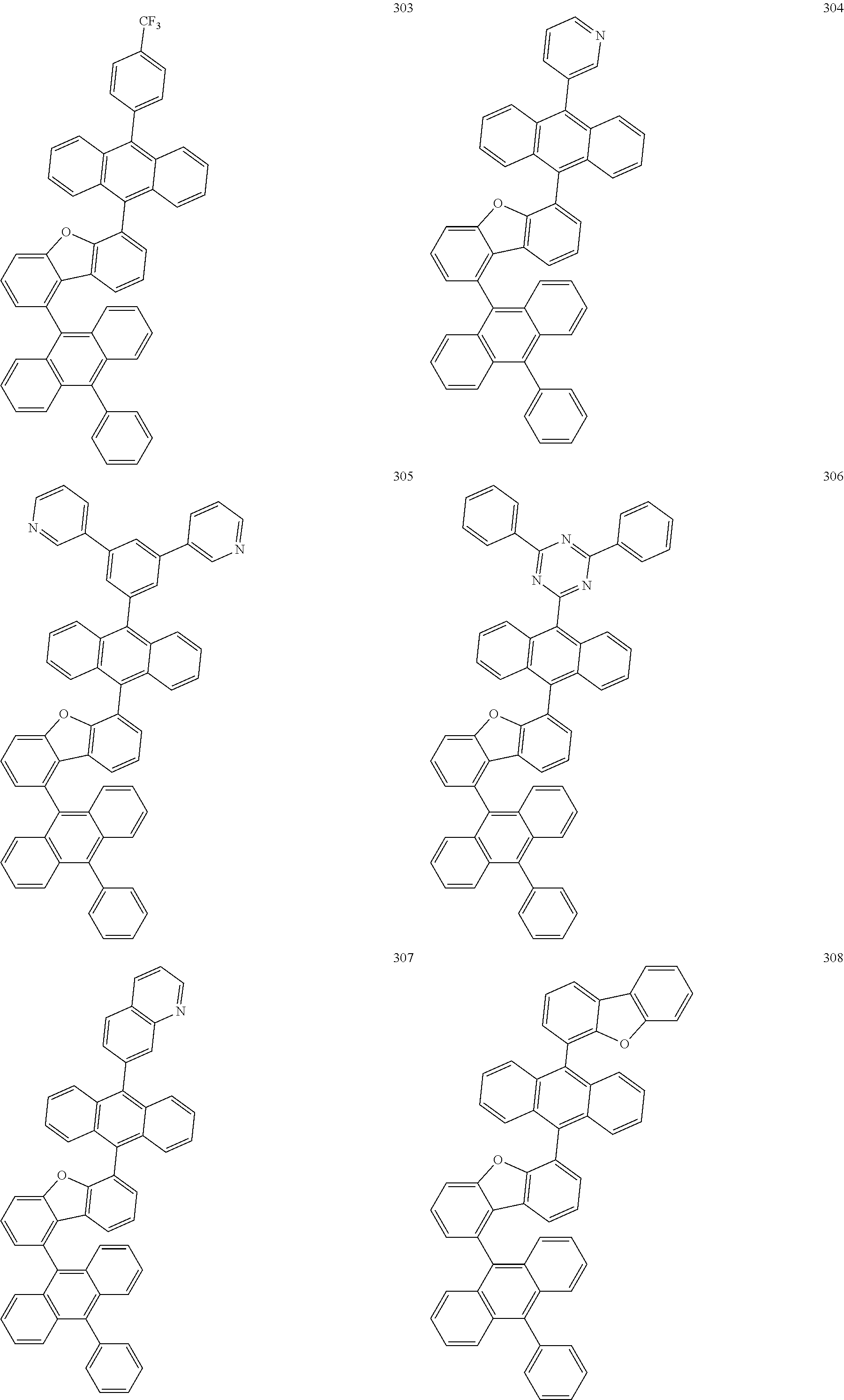

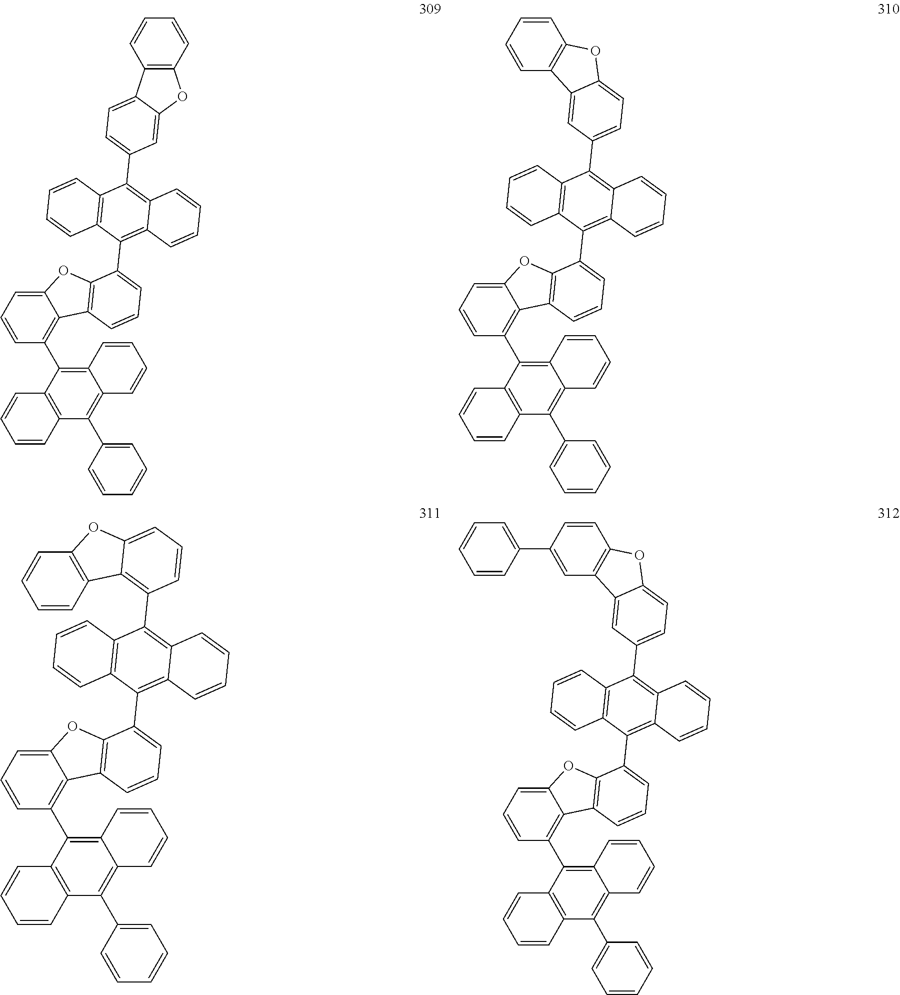

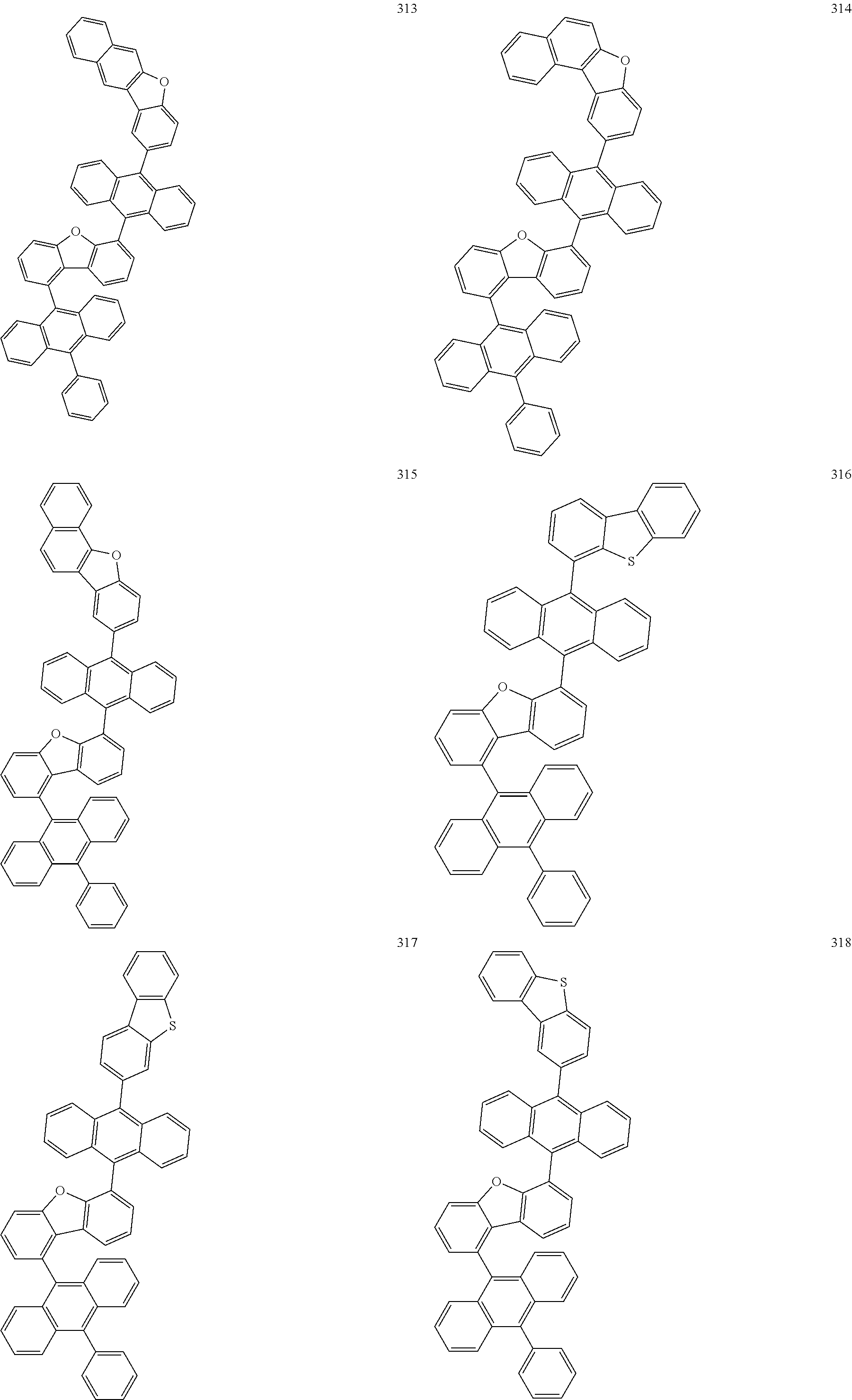

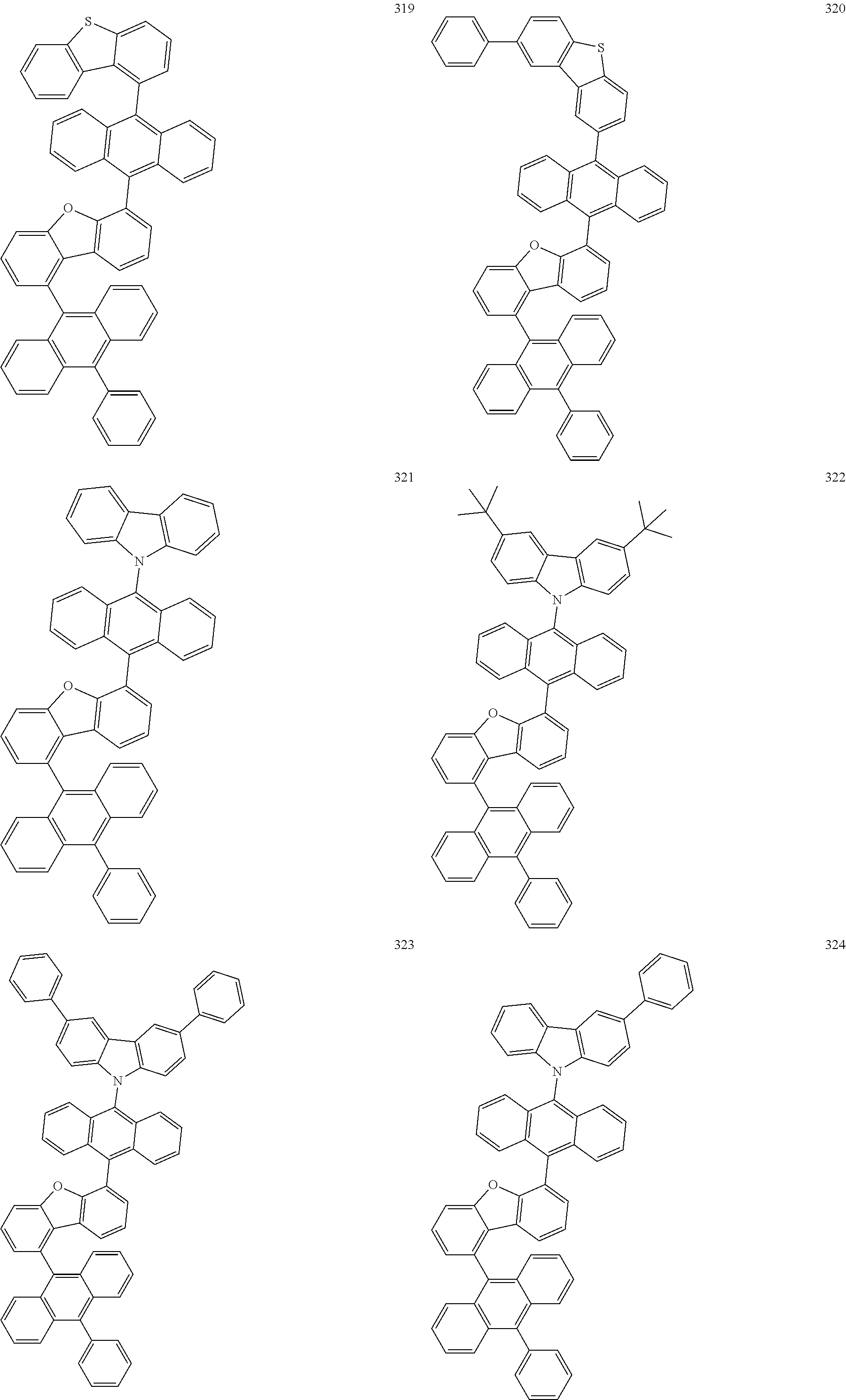

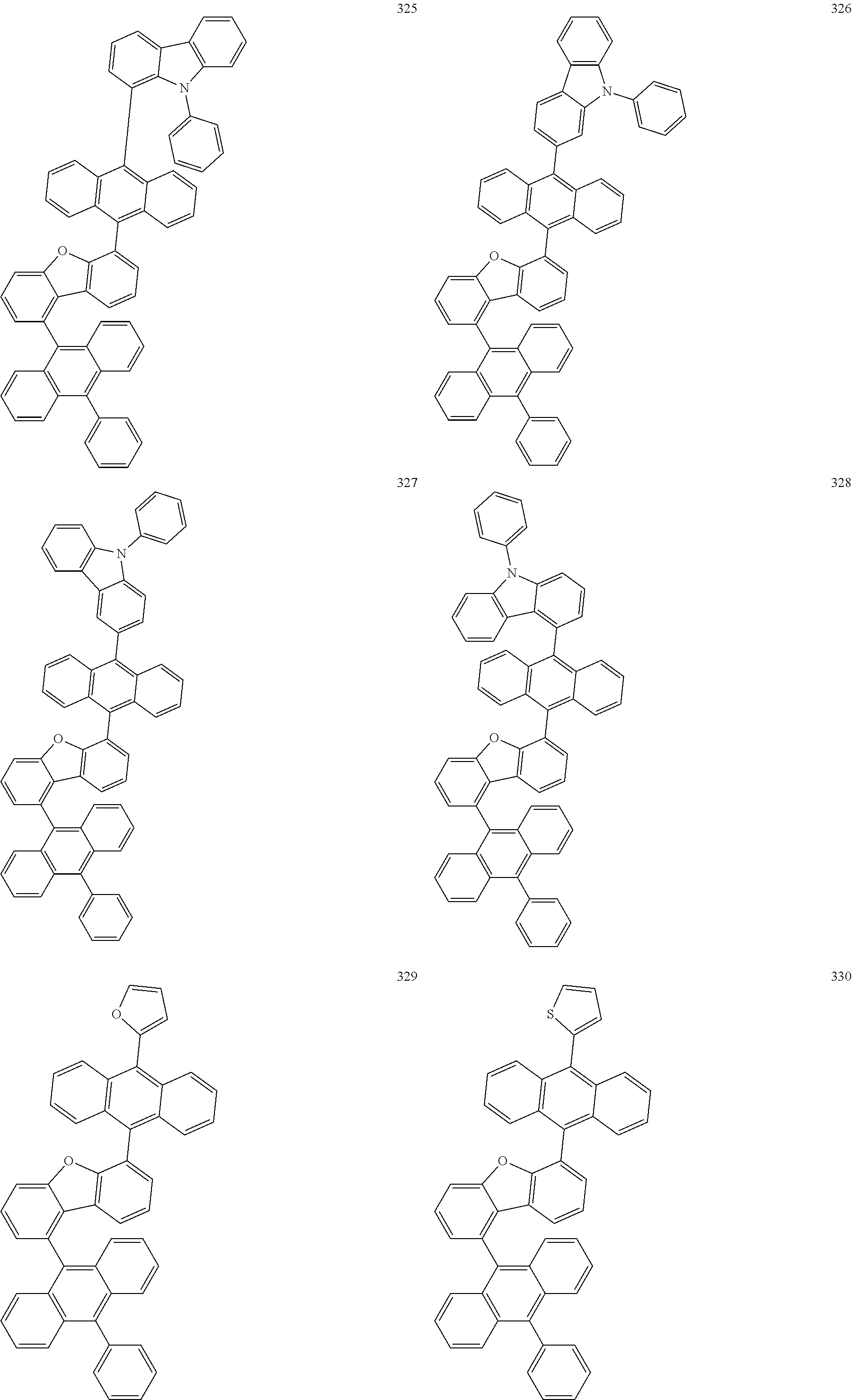

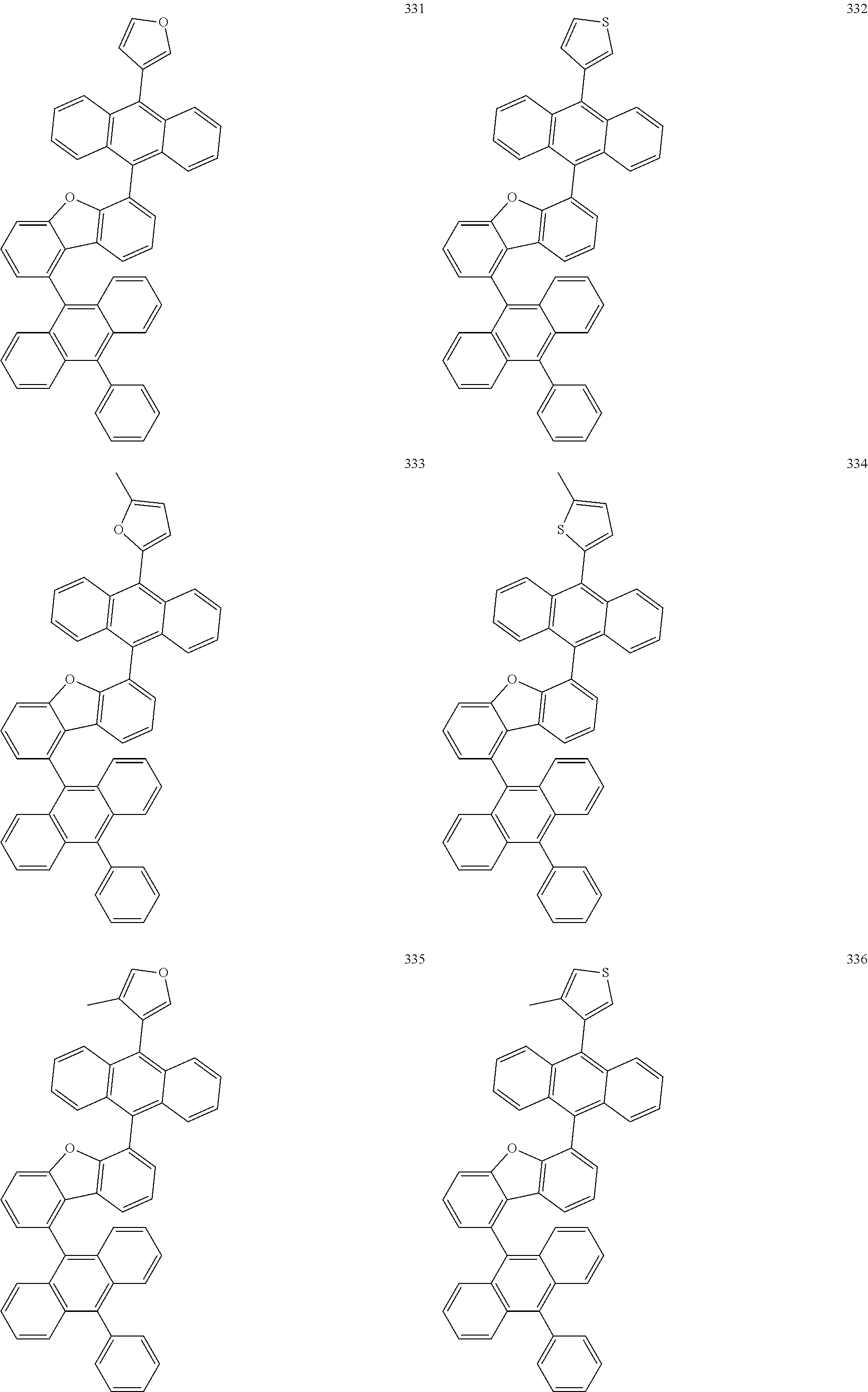

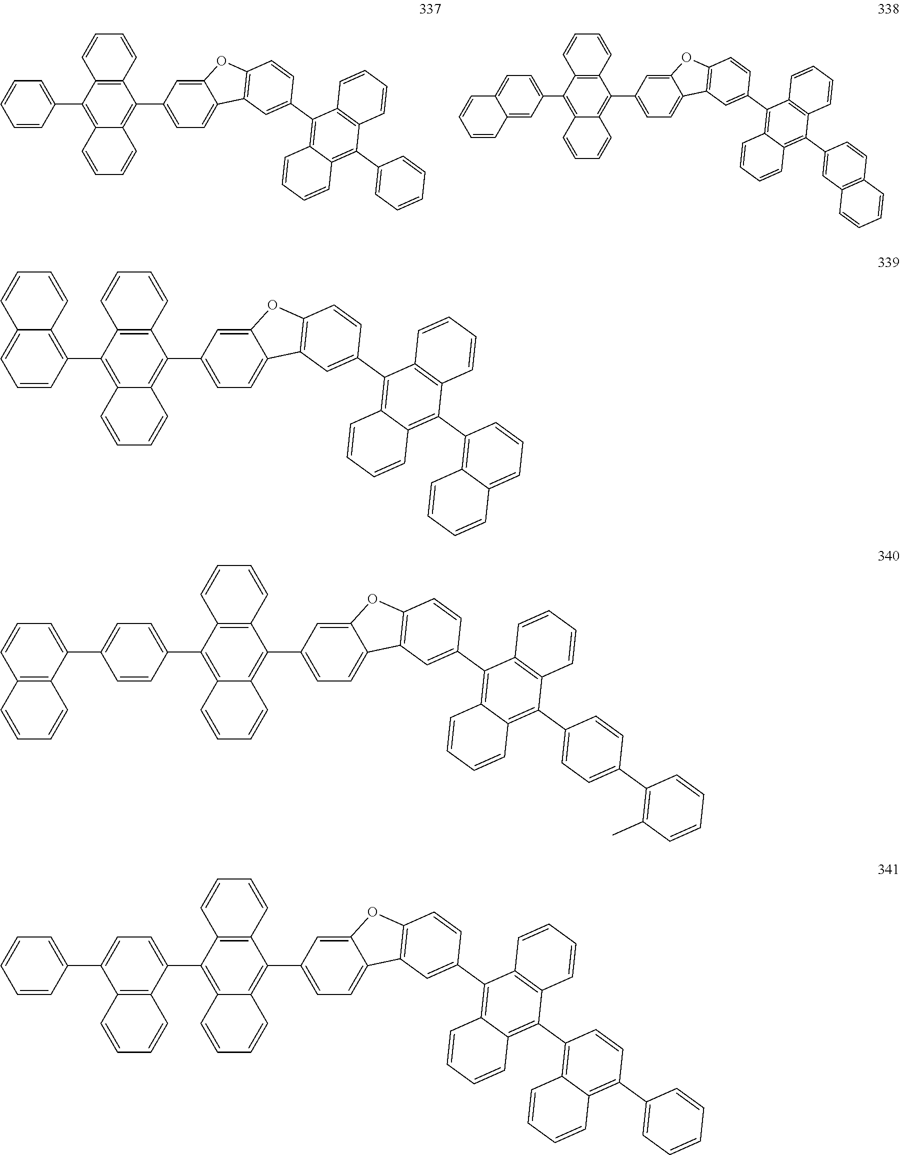

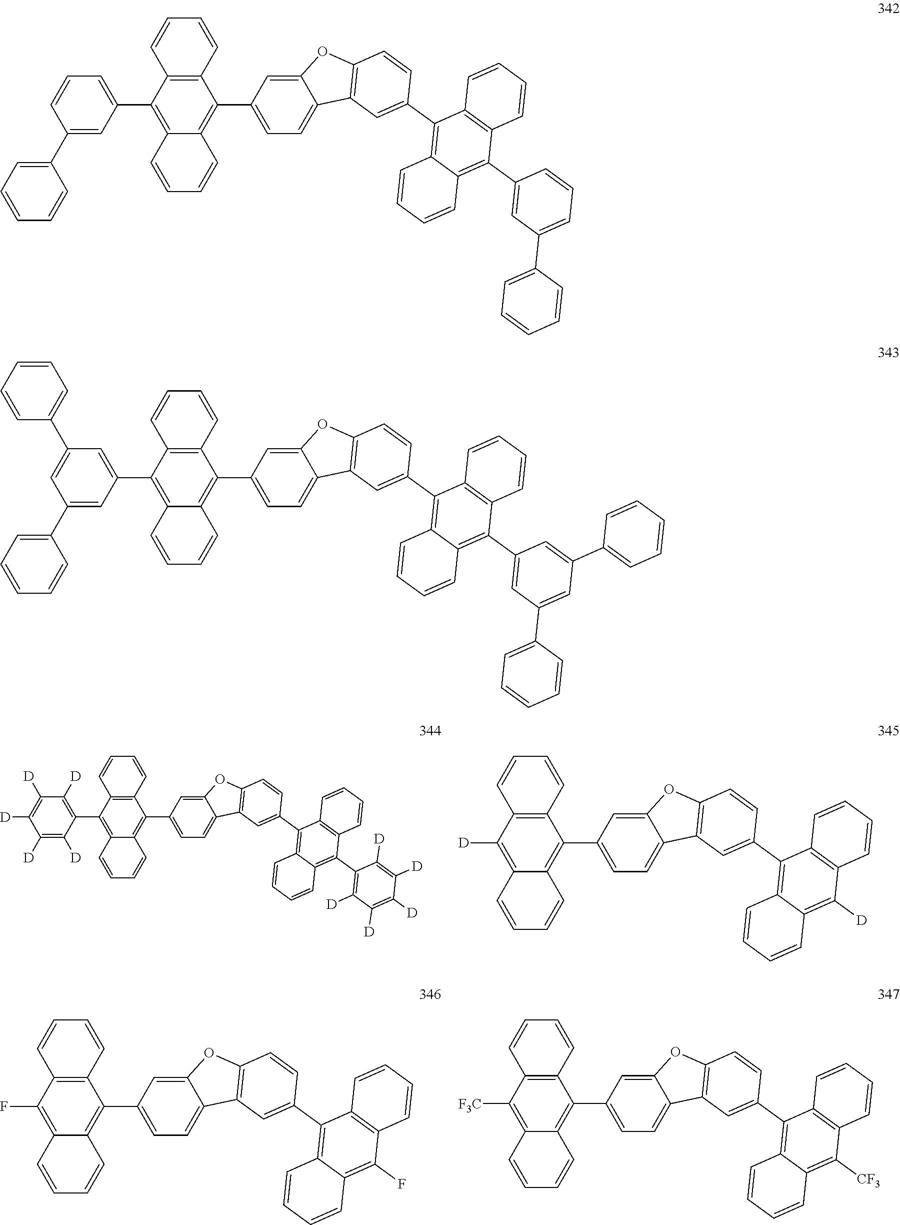

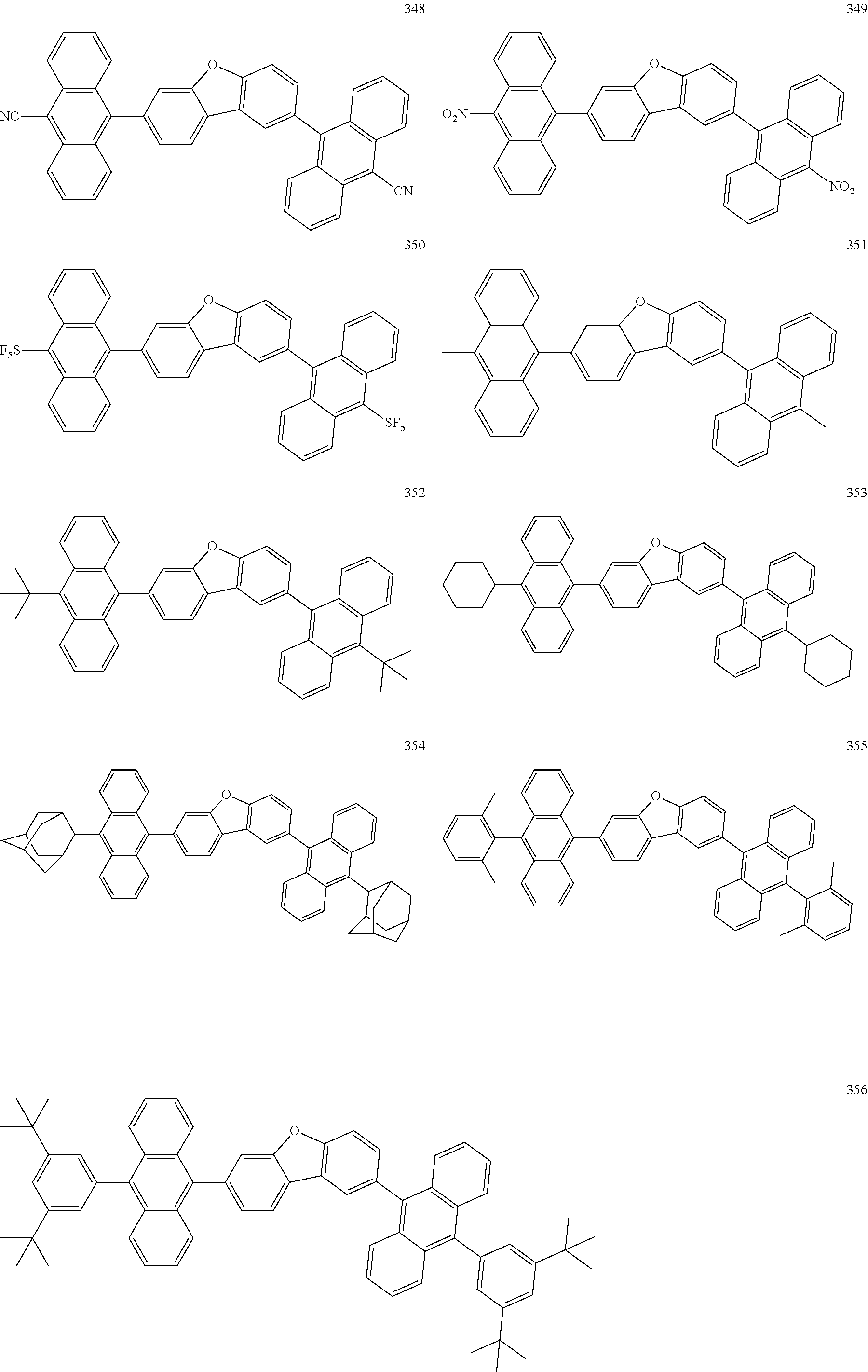

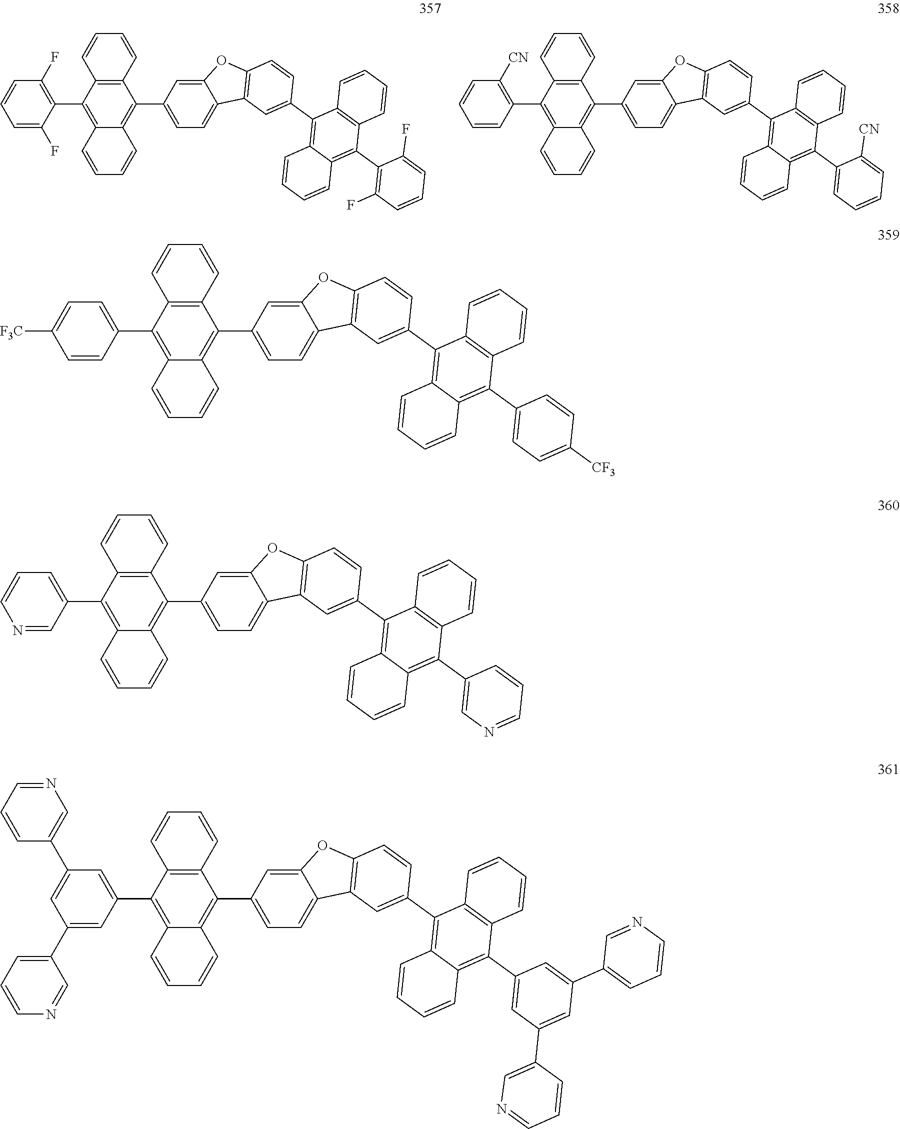

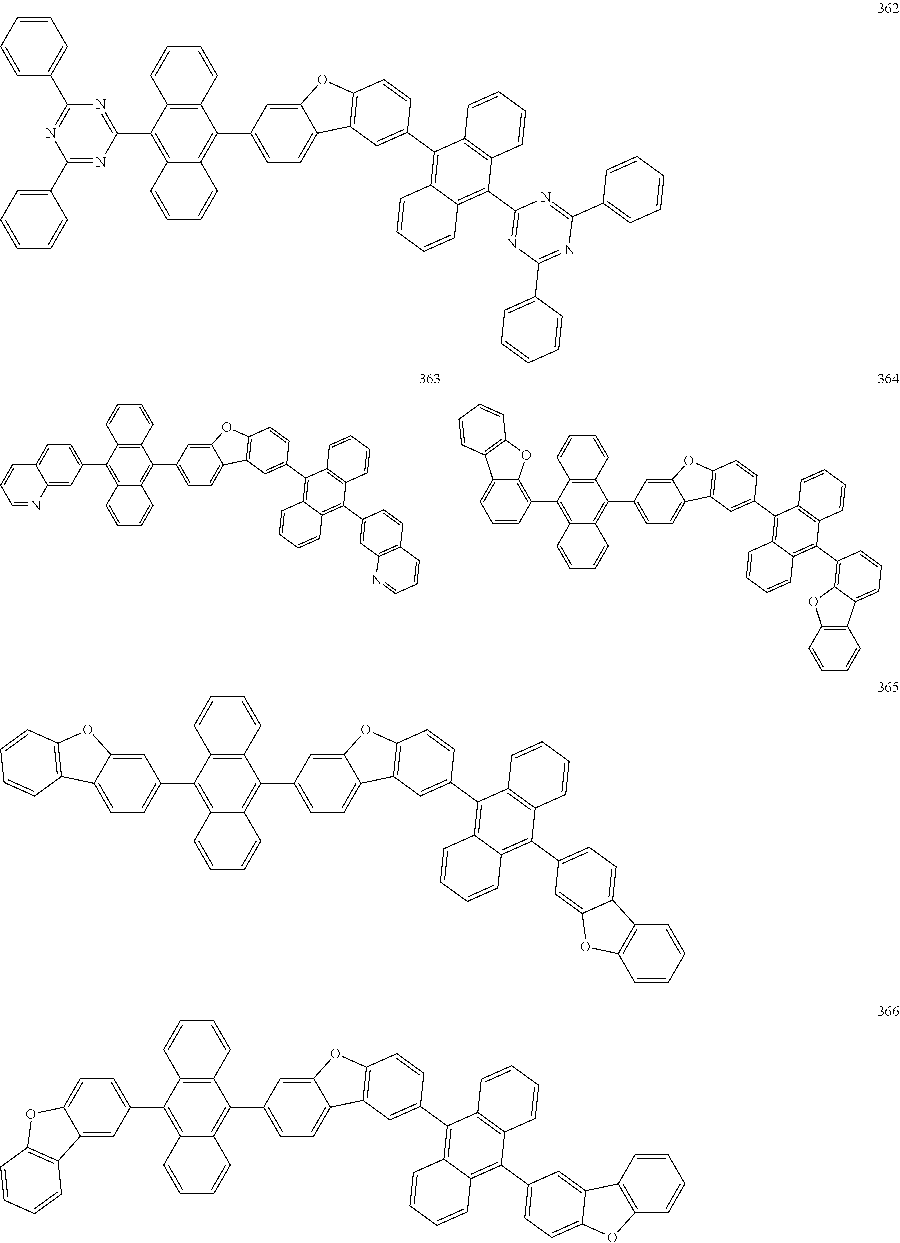

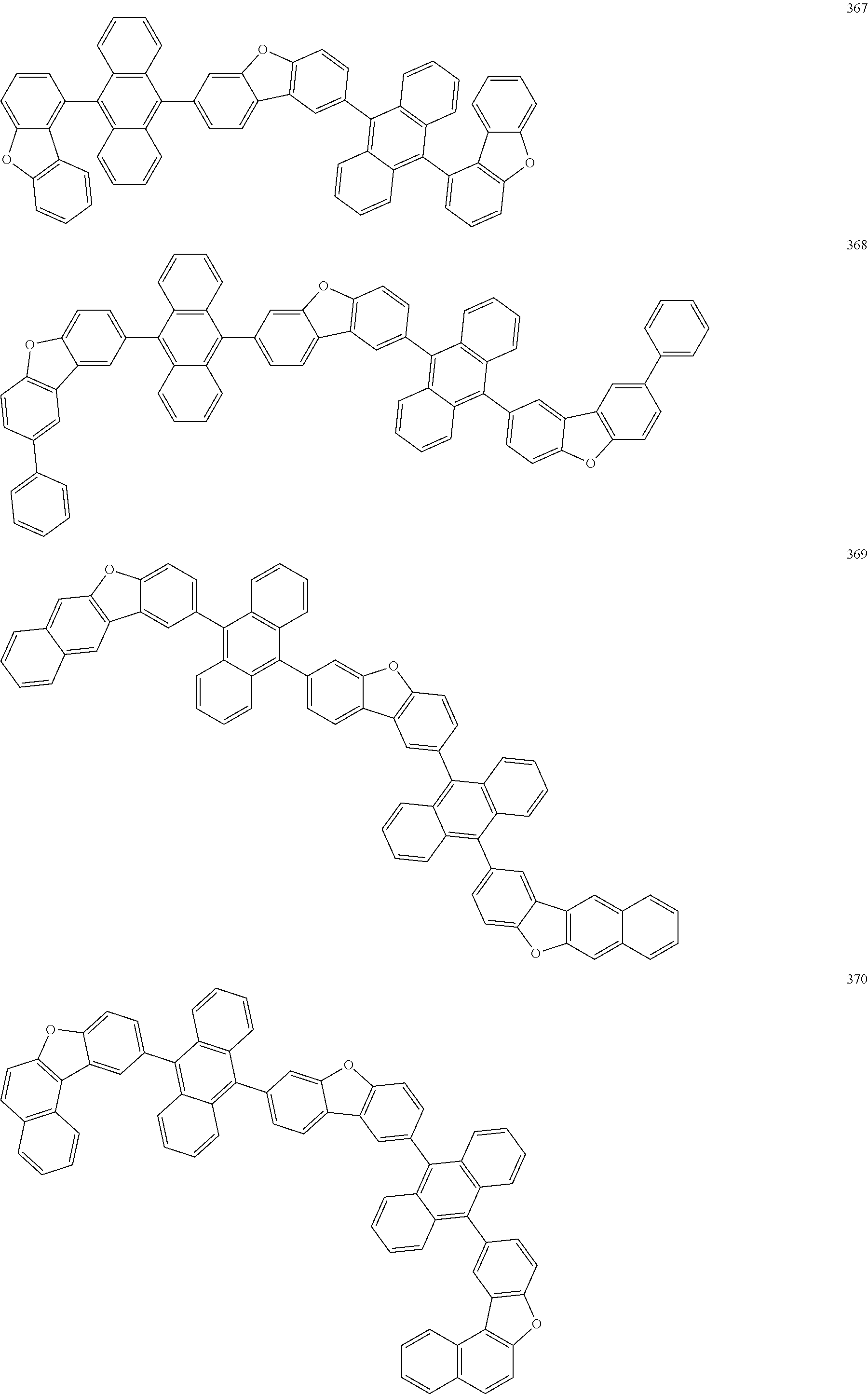

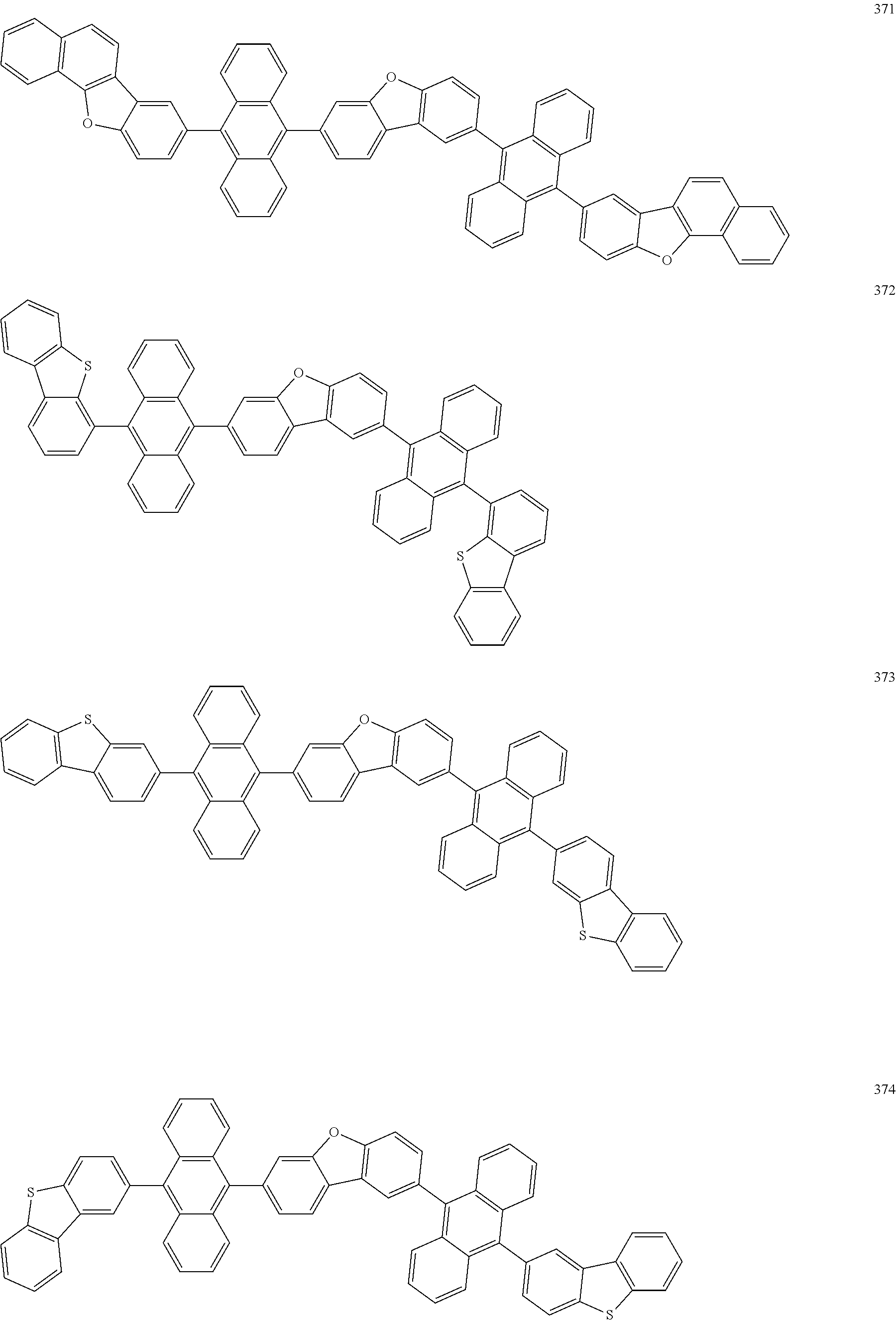 [SMILES: c1ccc2c(c1)ccc1c3cc(-c4c5ccccc5c(-c5ccc6c(c5)oc5ccc(-c7c8ccccc8c(-c8ccc9oc%10c%11ccccc%11ccc%10c9c8)c8ccccc78)cc56)c5ccccc45)ccc3oc21 ; c1ccc2c(c1)sc1c(-c3c4ccccc4c(-c4ccc5c(c4)oc4ccc(-c6c7ccccc7c(-c7cccc8c7sc7ccccc78)c7ccccc67)cc45)c4ccccc34)cccc12 ; c1ccc2c(c1)sc1cc(-c3c4ccccc4c(-c4ccc5c(c4)oc4ccc(-c6c7ccccc7c(-c7ccc8c(c7)sc7ccccc78)c7ccccc67)cc45)c4ccccc34)ccc12 ; c1ccc2c(c1)sc1ccc(-c3c4ccccc4c(-c4ccc5c(c4)oc4ccc(-c6c7ccccc7c(-c7ccc8sc9ccccc9c8c7)c7ccccc67)cc45)c4ccccc34)cc12]

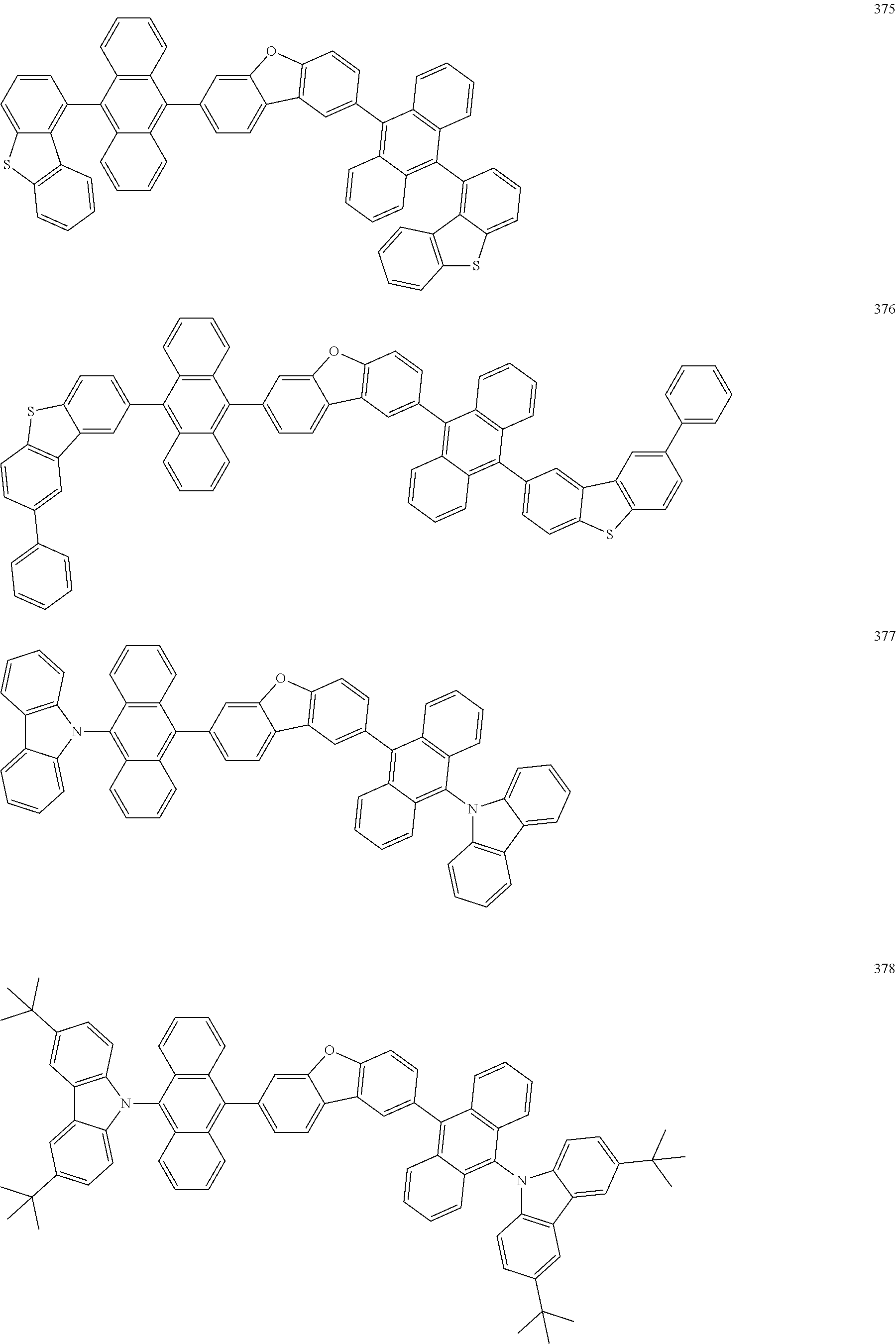

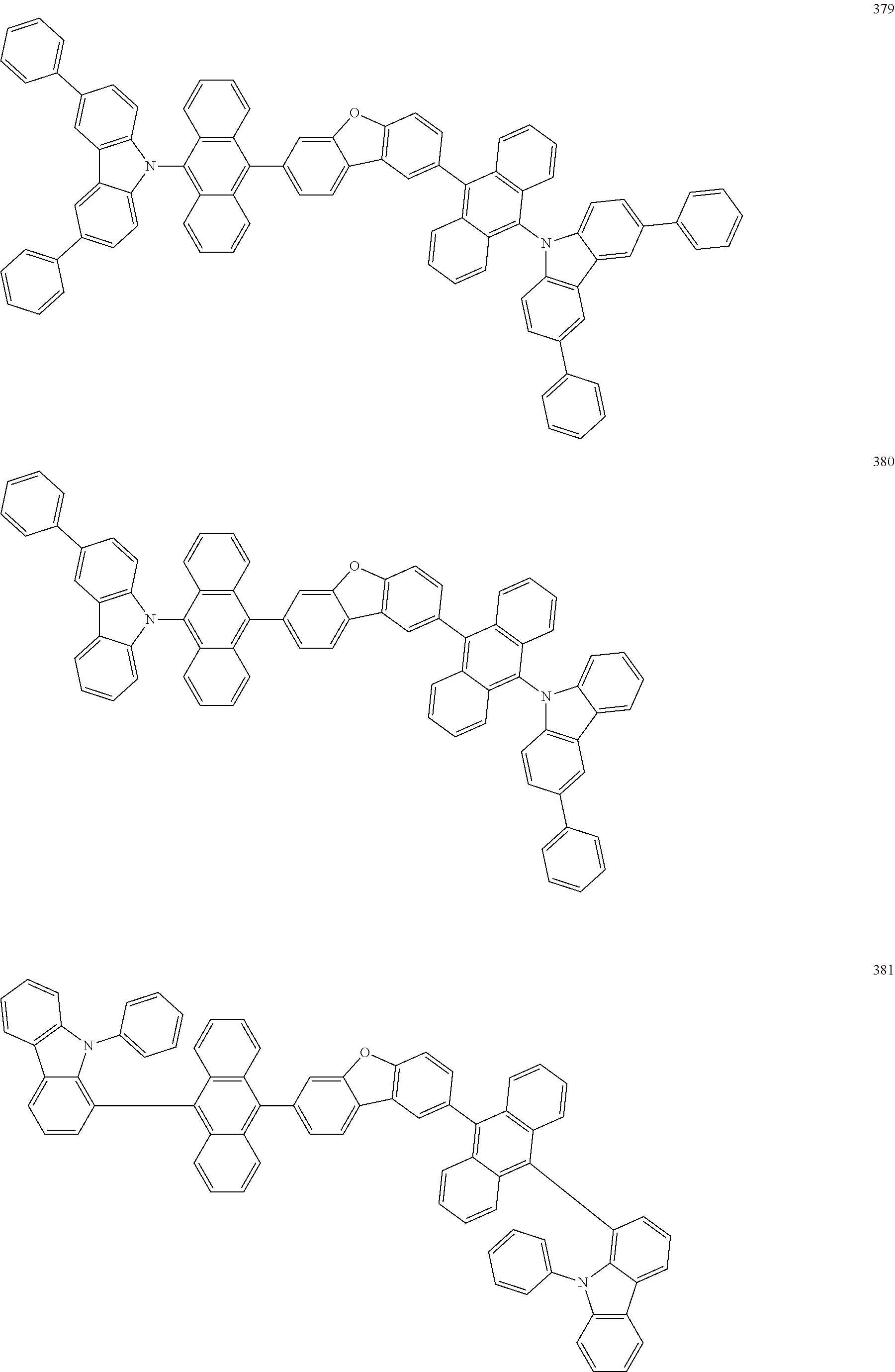 [SMILES: c1ccc(-c2ccc3c(c2)c2cc(-c4ccccc4)ccc2n3-c2c3ccccc3c(-c3ccc4c(c3)oc3ccc(-c5c6ccccc6c(-n6c7ccc(-c8ccccc8)cc7c7cc(-c8ccccc8)ccc76)c6ccccc56)cc34)c3ccccc23)cc1 ; c1ccc(-c2ccc3c(c2)c2ccccc2n3-c2c3ccccc3c(-c3ccc4c(c3)oc3ccc(-c5c6ccccc6c(-n6c7ccccc7c7cc(-c8ccccc8)ccc76)c6ccccc56)cc34)c3ccccc23)cc1 ; c1ccc(-n2c3ccccc3c3cccc(-c4c5ccccc5c(-c5ccc6c(c5)oc5ccc(-c7c8ccccc8c(-c8cccc9c%10ccccc%10n(-c%10ccccc%10)c89)c8ccccc78)cc56)c5ccccc45)c32)cc1]

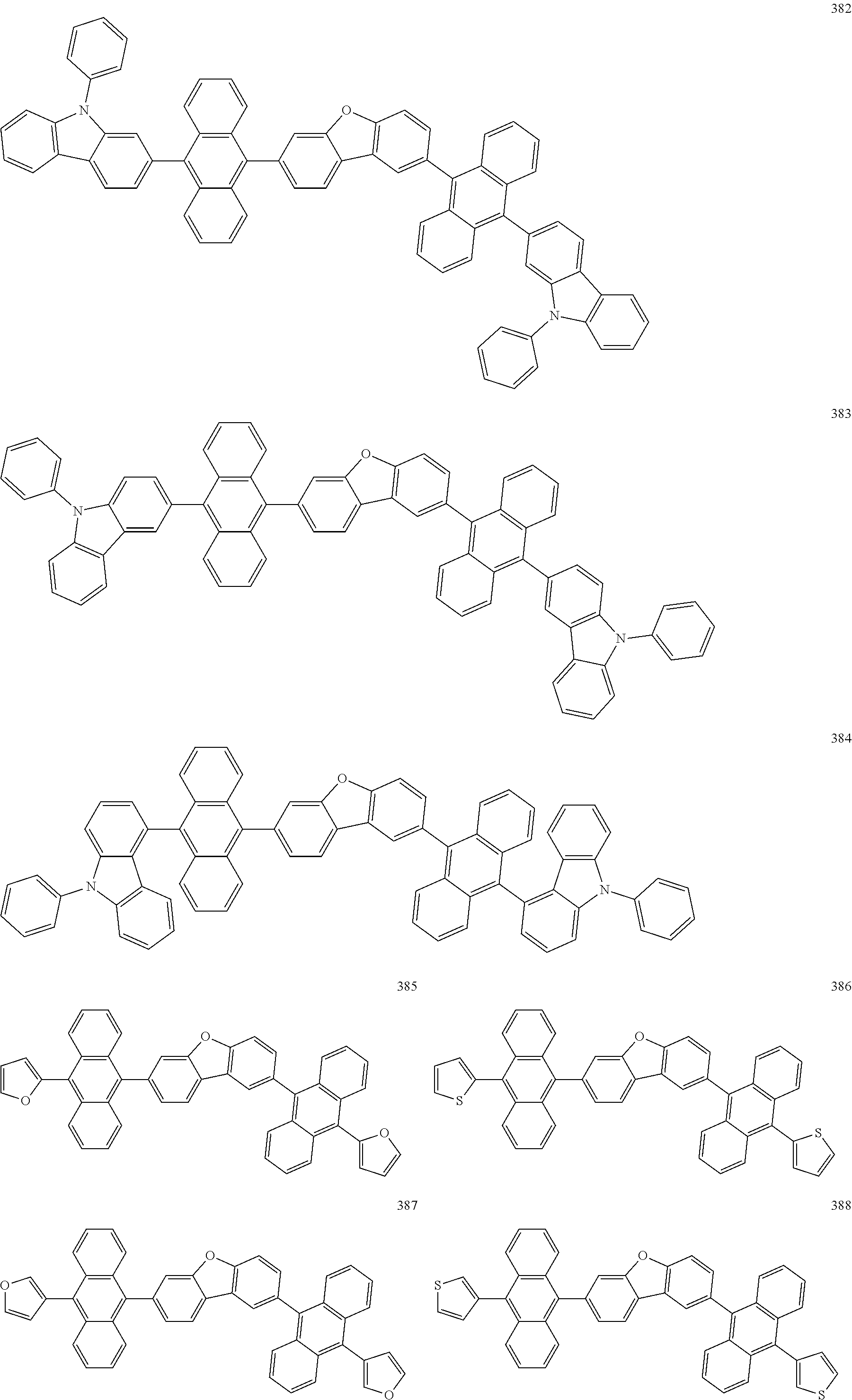

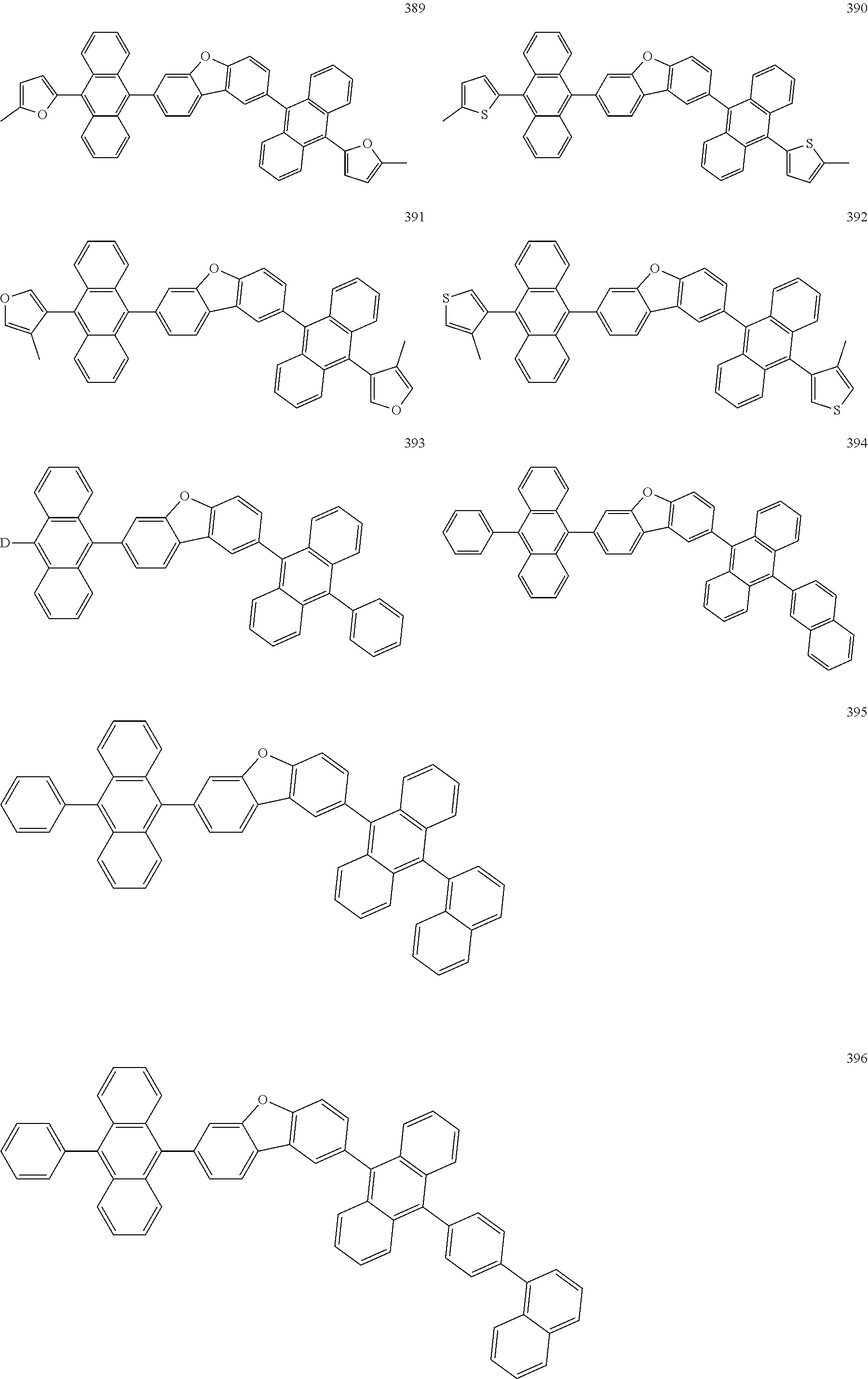

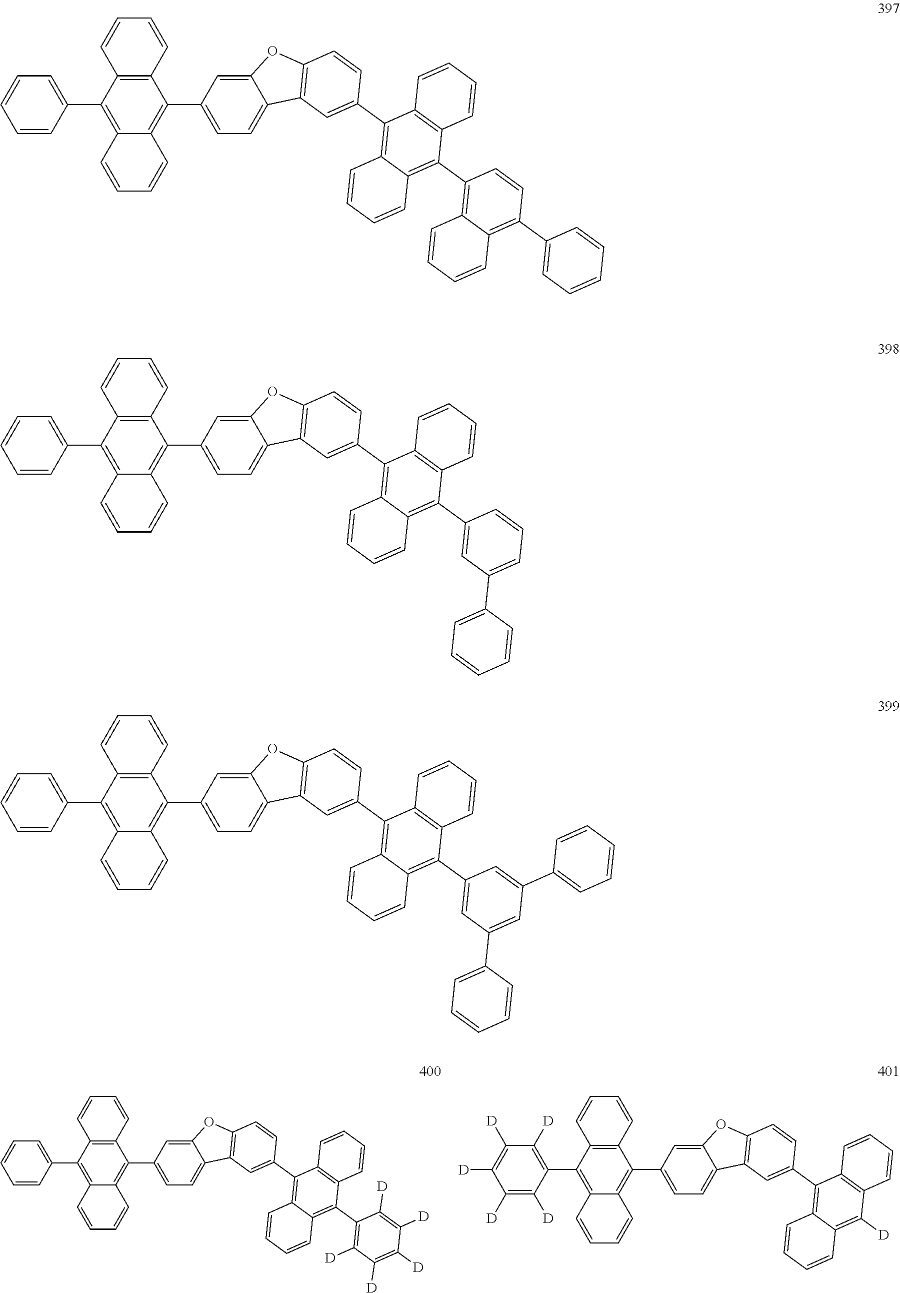

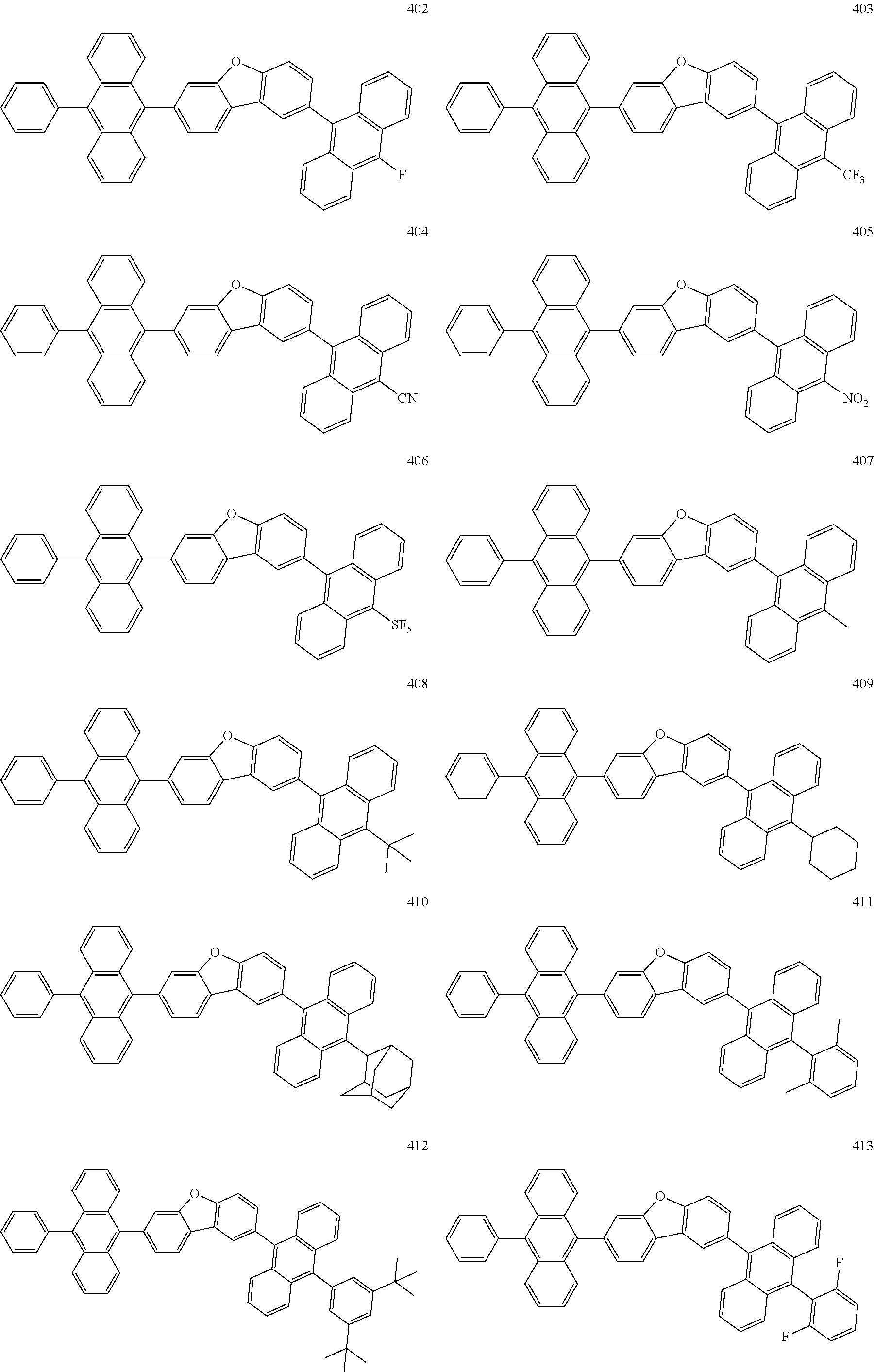

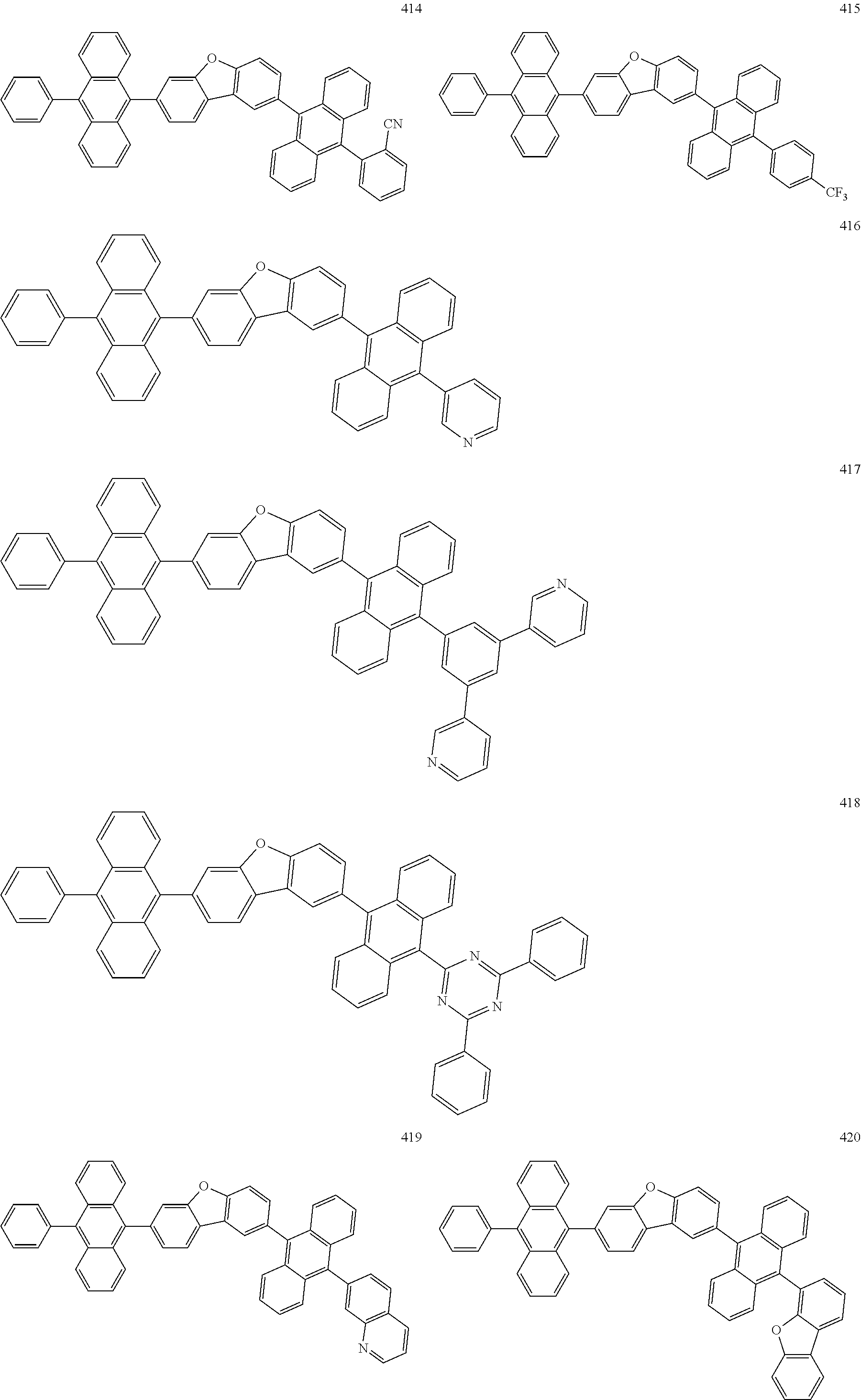

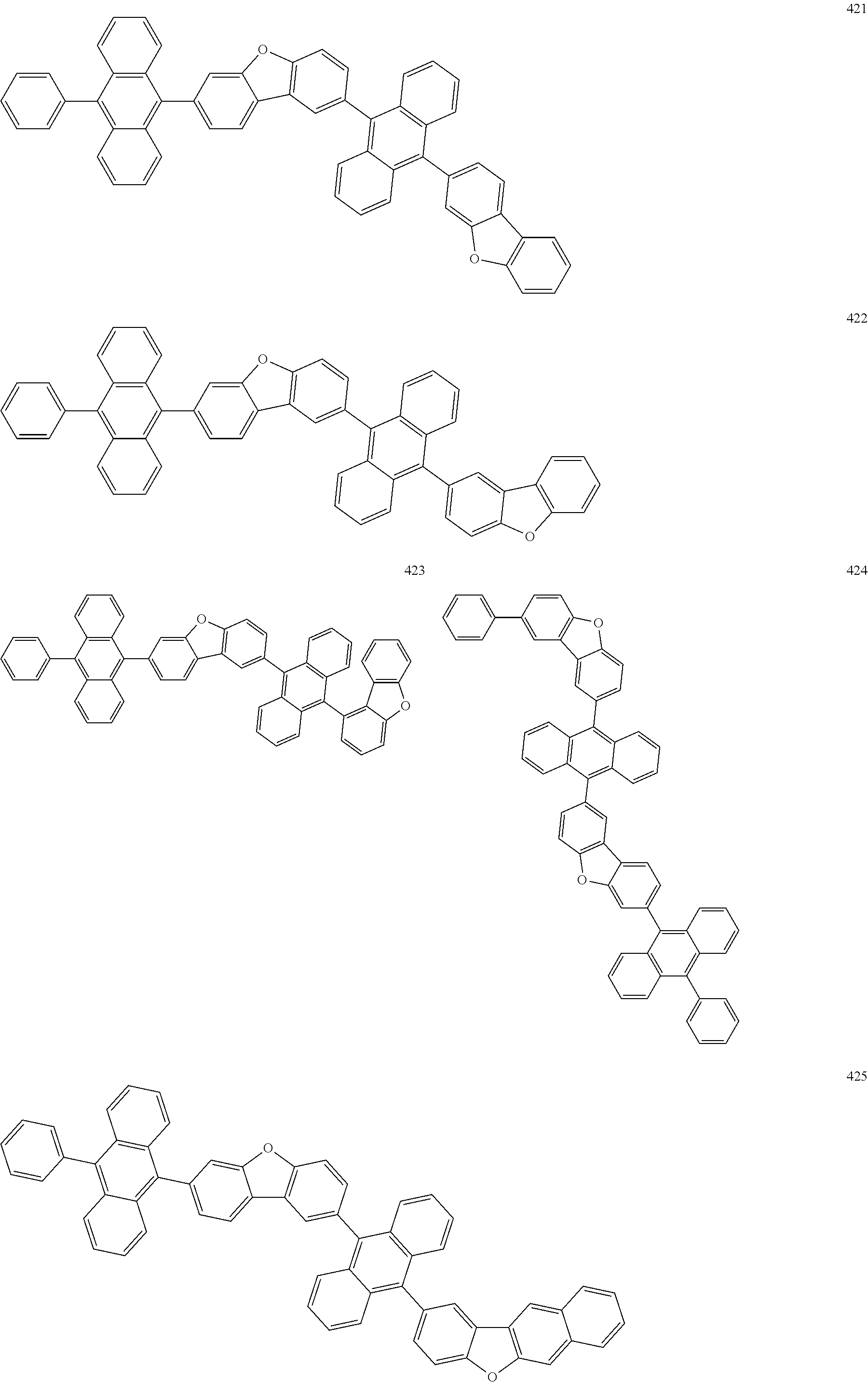

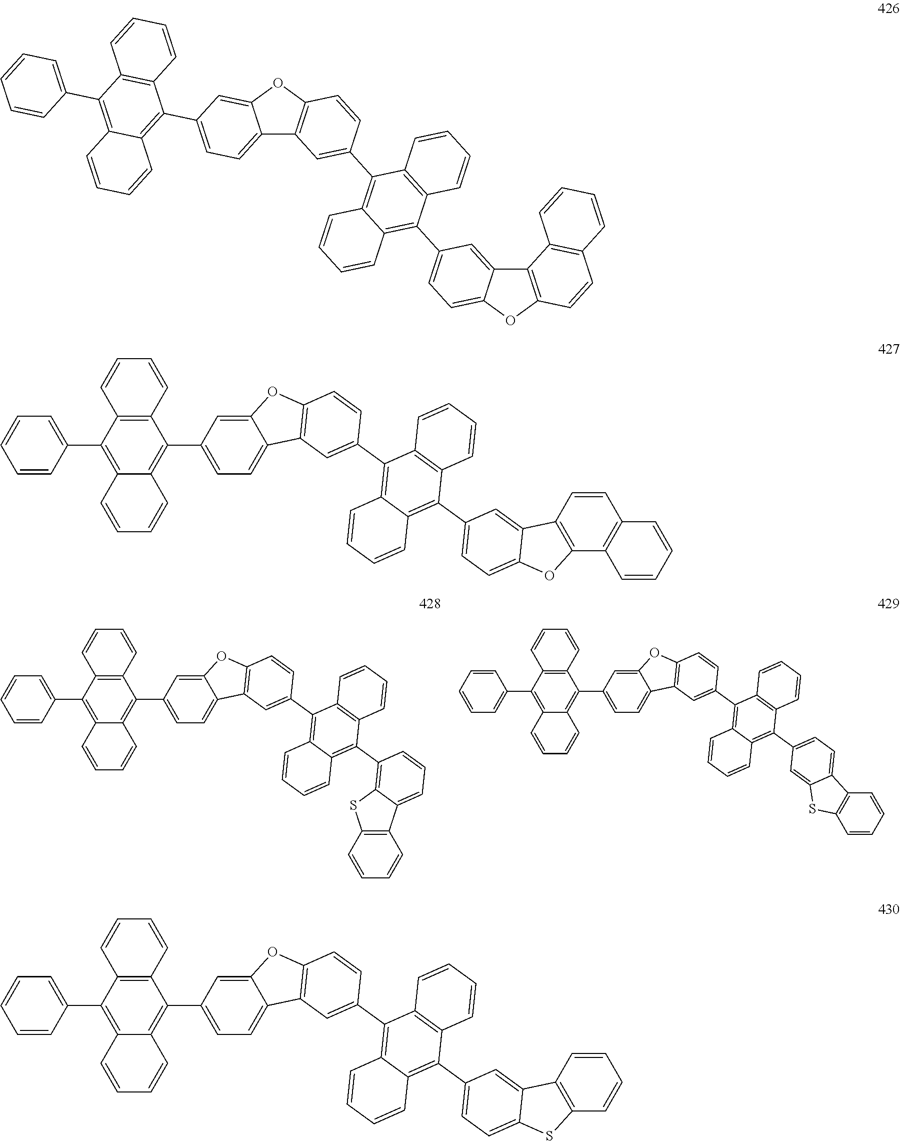

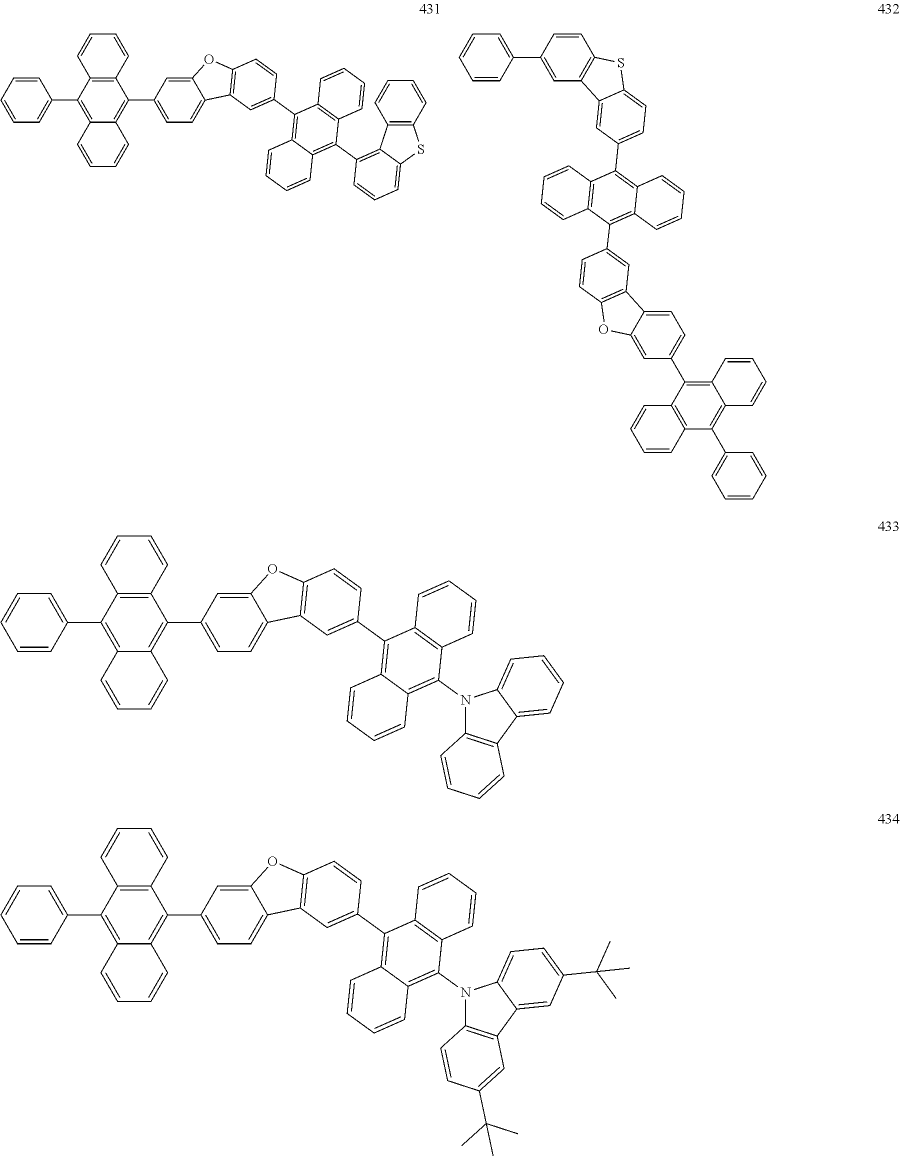 [SMILES: c1ccc(-c2c3ccccc3c(-c3ccc4c(c3)oc3ccc(-c5c6ccccc6c(-c6cccc7sc8ccccc8c67)c6ccccc56)cc34)c3ccccc23)cc1 ; c1ccc(-c2ccc3sc4ccc(-c5c6ccccc6c(-c6ccc7oc8cc(-c9c%10ccccc%10c(-c%10ccccc%10)c%10ccccc9%10)ccc8c7c6)c6ccccc56)cc4c3c2)cc1 ; c1ccc(-c2c3ccccc3c(-c3ccc4c(c3)oc3ccc(-c5c6ccccc6c(-n6c7ccccc7c7ccccc76)c6ccccc56)cc34)c3ccccc23)cc1 ; CC(C)(C)c1ccc2c(c1)c1cc(C(C)(C)C)ccc1n2-c1c2ccccc2c(-c2ccc3oc4cc(-c5c6ccccc6c(-c6ccccc6)c6ccccc56)ccc4c3c2)c2ccccc12]

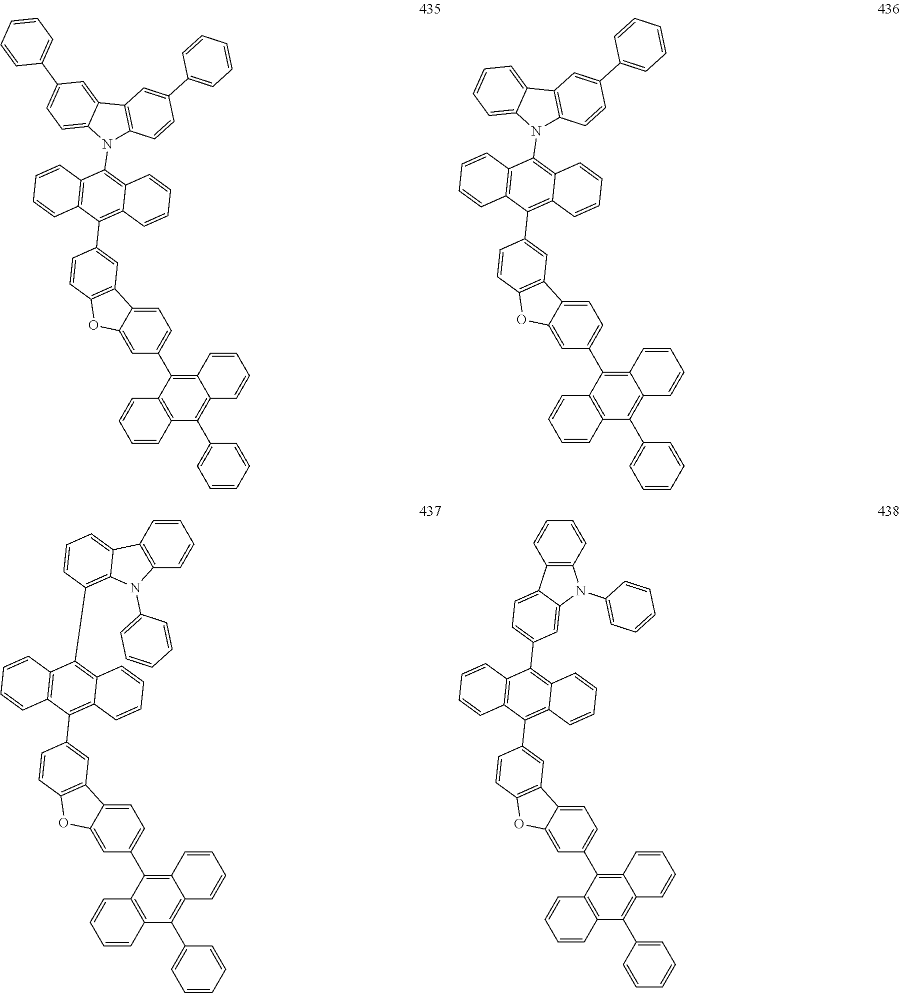 [SMILES: c1ccc(-c2ccc3c(c2)c2cc(-c4ccccc4)ccc2n3-c2c3ccccc3c(-c3ccc4oc5cc(-c6c7ccccc7c(-c7ccccc7)c7ccccc67)ccc5c4c3)c3ccccc23)cc1 ; c1ccc(-c2ccc3c(c2)c2ccccc2n3-c2c3ccccc3c(-c3ccc4oc5cc(-c6c7ccccc7c(-c7ccccc7)c7ccccc67)ccc5c4c3)c3ccccc23)cc1 ; c1ccc(-c2c3ccccc3c(-c3ccc4c(c3)oc3ccc(-c5c6ccccc6c(-c6cccc7c8ccccc8n(-c8ccccc8)c67)c6ccccc56)cc34)c3ccccc23)cc1 ; c1ccc(-c2c3ccccc3c(-c3ccc4c(c3)oc3ccc(-c5c6ccccc6c(-c6ccc7c8ccccc8n(-c8ccccc8)c7c6)c6ccccc56)cc34)c3ccccc23)cc1]

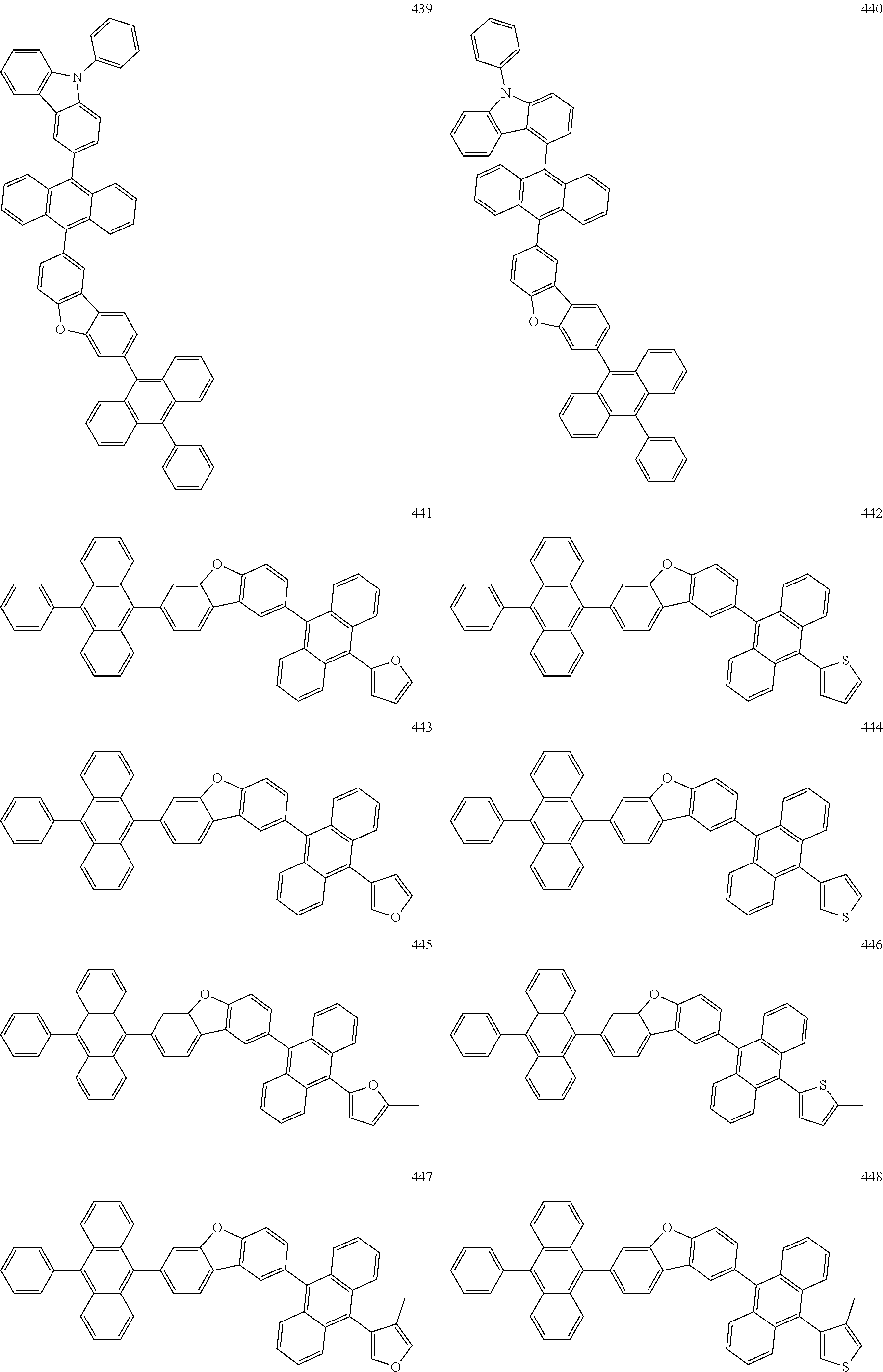

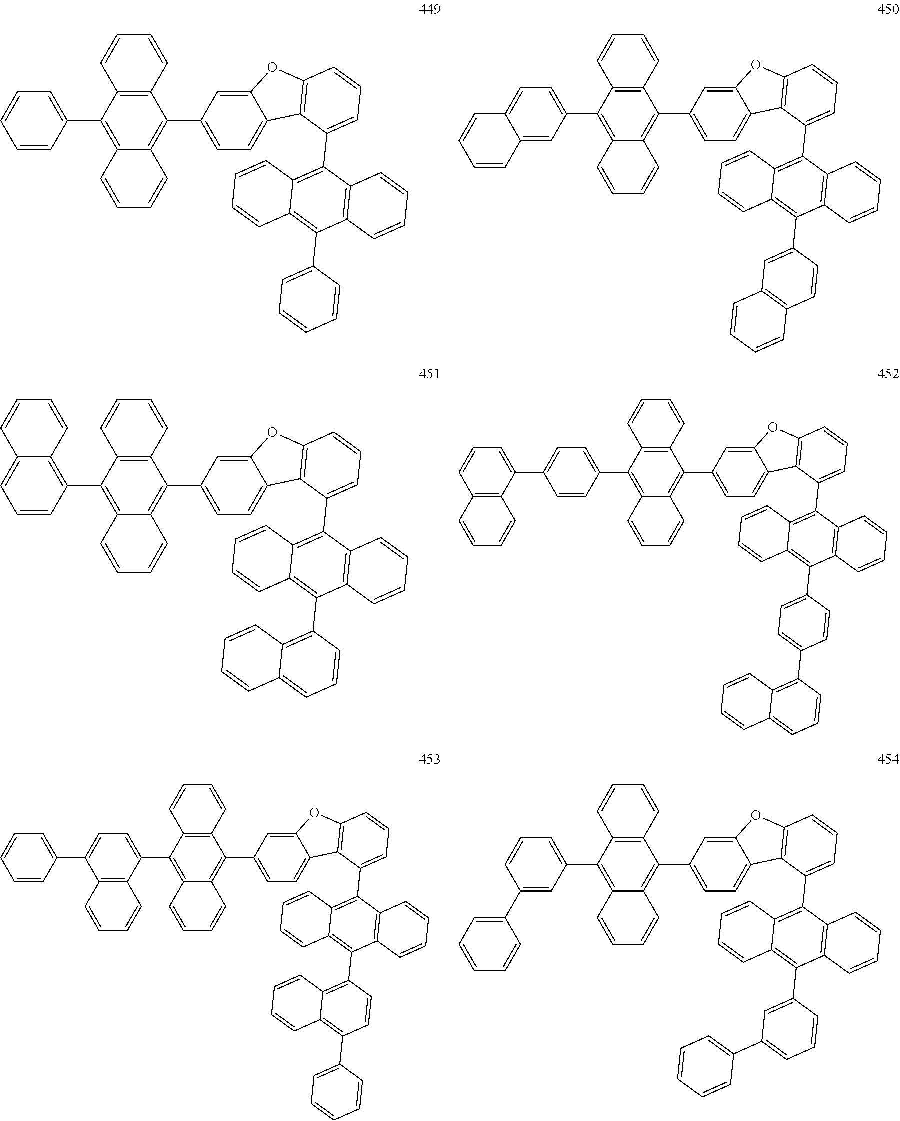

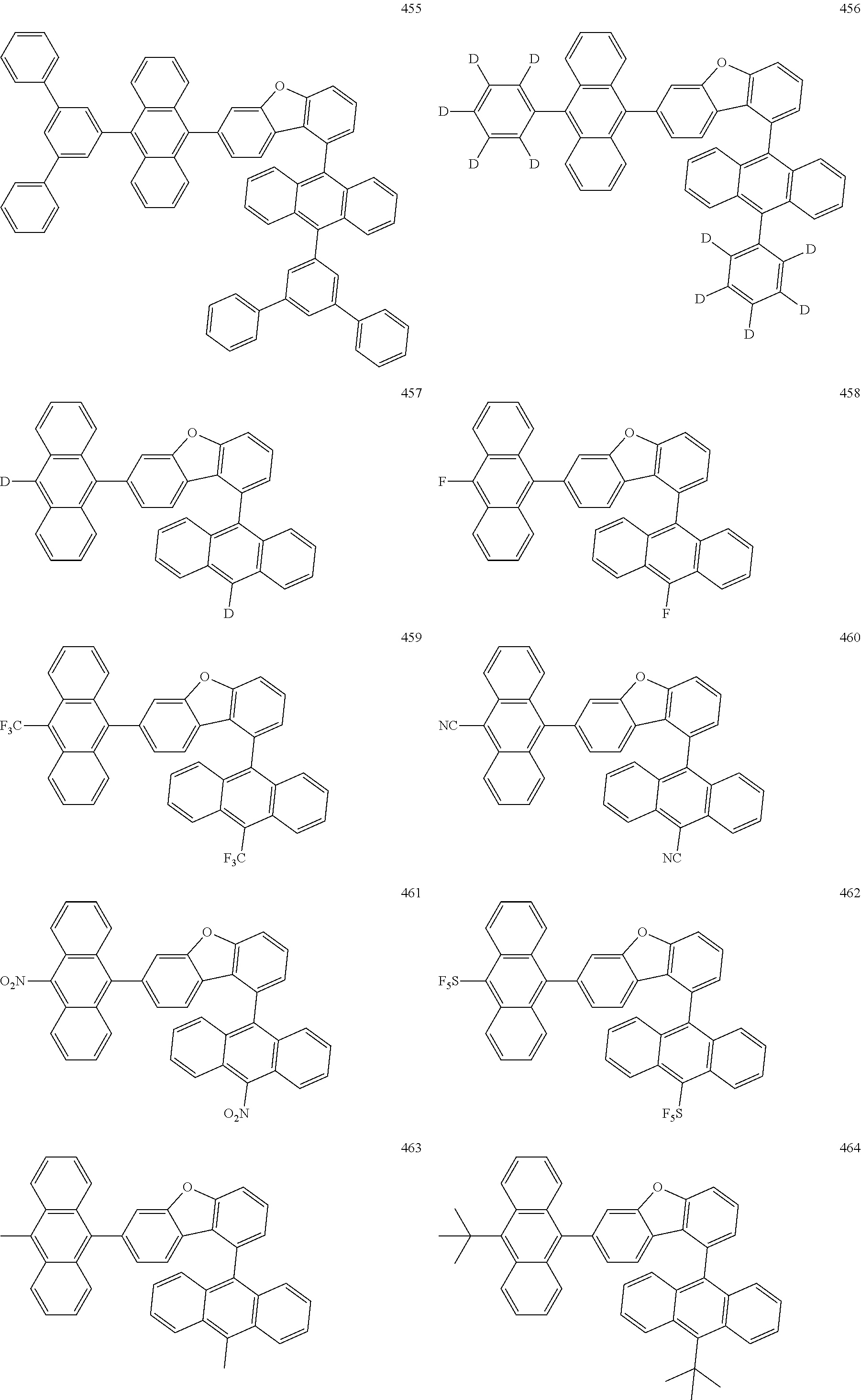

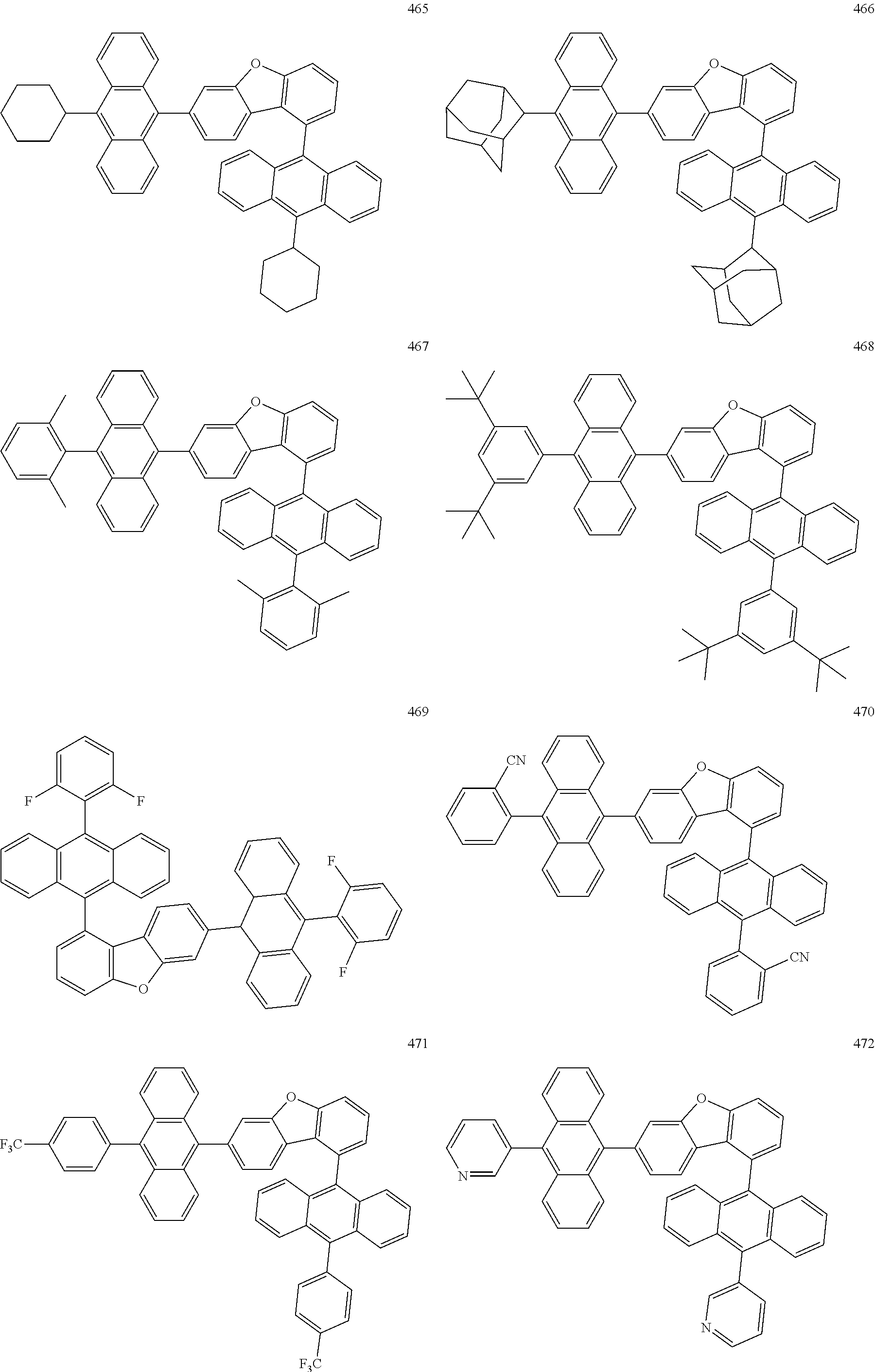

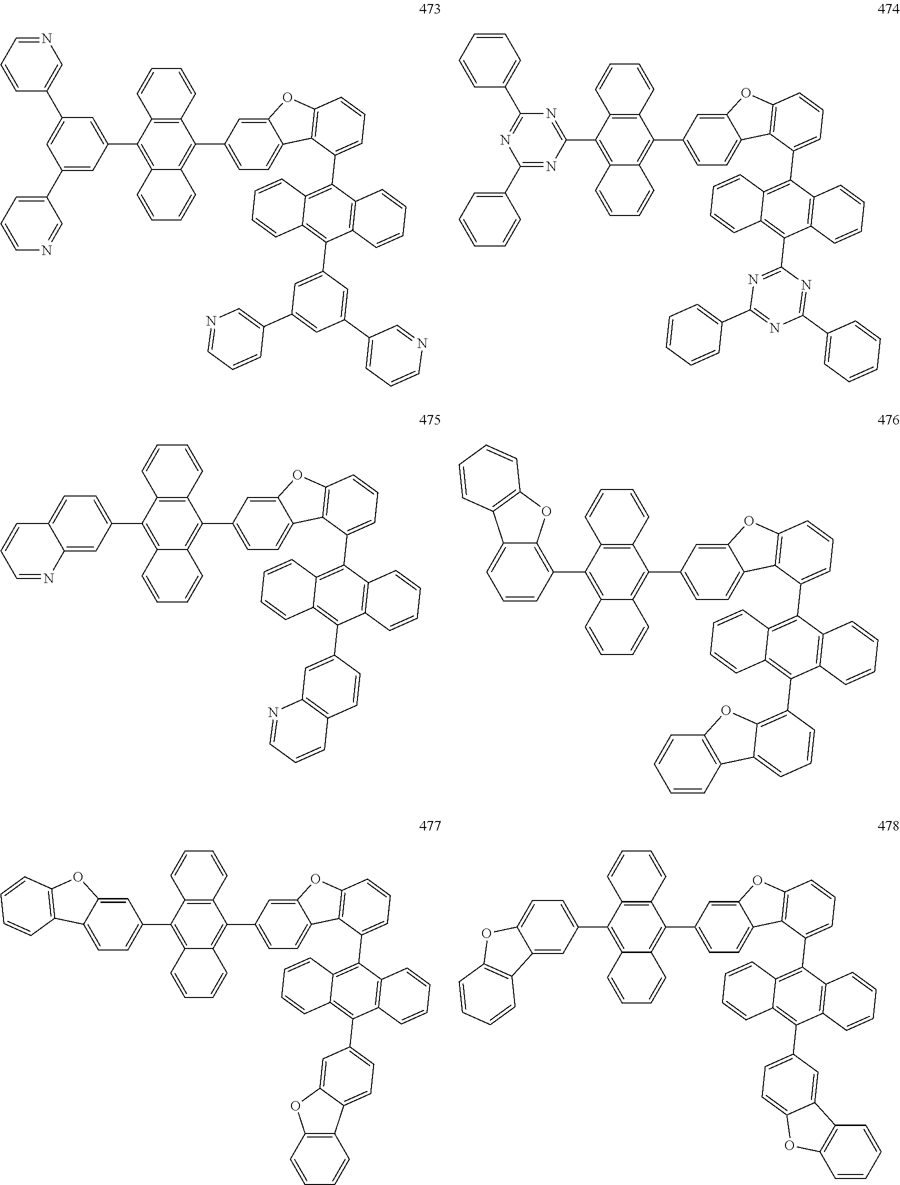

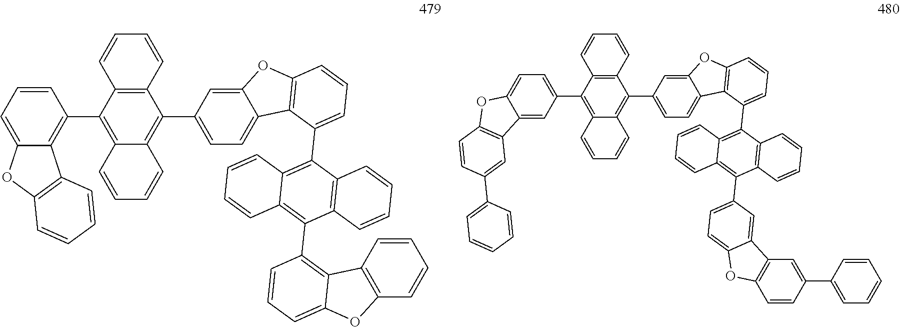 [SMILES: c1ccc2c(c1)oc1cccc(-c3c4ccccc4c(-c4ccc5c(c4)oc4cccc(-c6c7ccccc7c(-c7cccc8oc9ccccc9c78)c7ccccc67)c45)c4ccccc34)c12 ; c1ccc(-c2ccc3oc4ccc(-c5c6ccccc6c(-c6ccc7c(c6)oc6cccc(-c8c9ccccc9c(-c9ccc%10oc%11ccc(-c%12ccccc%12)cc%11c%10c9)c9ccccc89)c67)c6ccccc56)cc4c3c2)cc1]

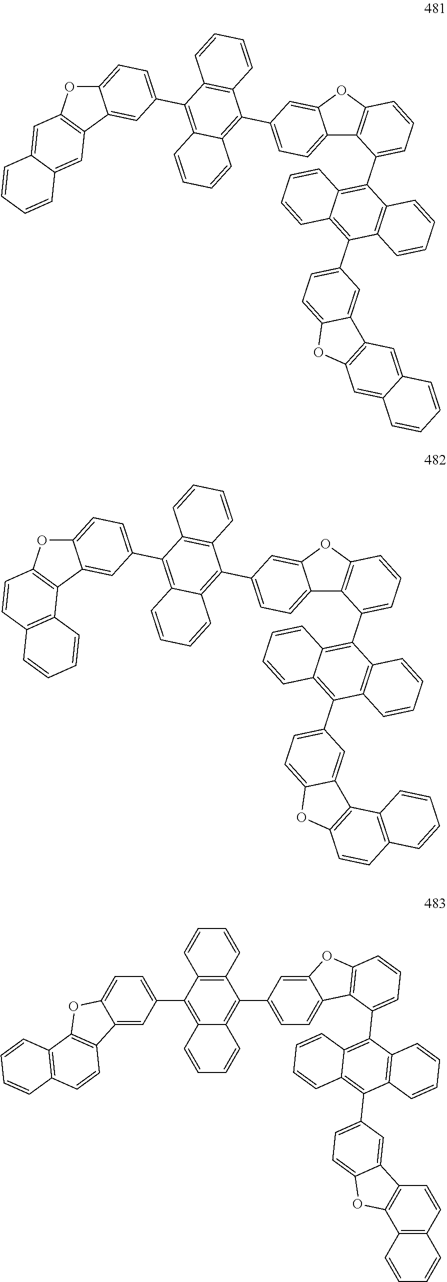 [SMILES: c1ccc2cc3c(cc2c1)oc1ccc(-c2c4ccccc4c(-c4ccc5c(c4)oc4cccc(-c6c7ccccc7c(-c7ccc8oc9cc%10ccccc%10cc9c8c7)c7ccccc67)c45)c4ccccc24)cc13 ; c1ccc2c(c1)ccc1oc3ccc(-c4c5ccccc5c(-c5ccc6c(c5)oc5cccc(-c7c8ccccc8c(-c8ccc9oc%10ccc%11ccccc%11c%10c9c8)c8ccccc78)c56)c5ccccc45)cc3c12 ; c1ccc2c(c1)ccc1c3cc(-c4c5ccccc5c(-c5ccc6c(c5)oc5cccc(-c7c8ccccc8c(-c8ccc9oc%10c%11ccccc%11ccc%10c9c8)c8ccccc78)c56)c5ccccc45)ccc3oc21]

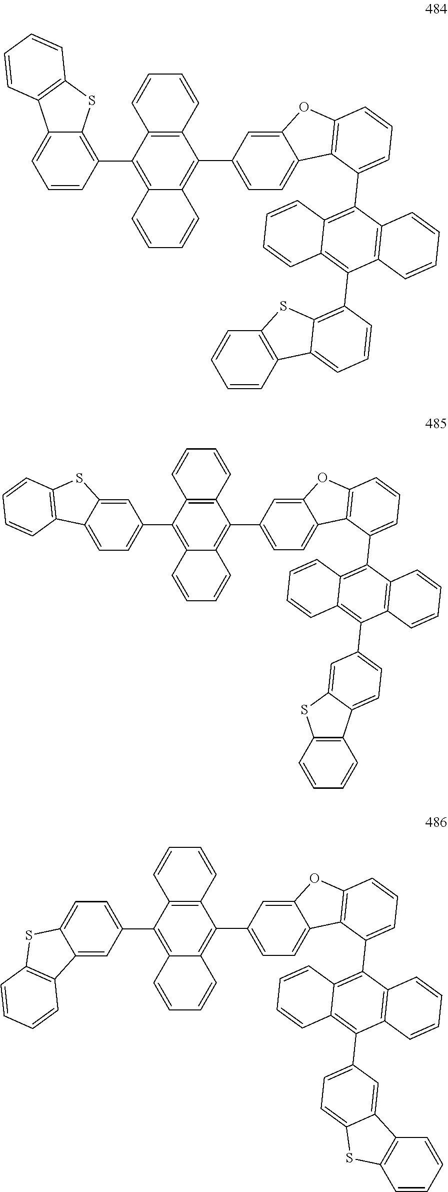 [SMILES: c1cc(-c2c3ccccc3c(-c3cccc4c3sc3ccccc34)c3ccccc23)c2c(c1)oc1cc(-c3c4ccccc4c(-c4cccc5c4sc4ccccc45)c4ccccc34)ccc12 ; c1cc(-c2c3ccccc3c(-c3ccc4c(c3)sc3ccccc34)c3ccccc23)c2c(c1)oc1cc(-c3c4ccccc4c(-c4ccc5c(c4)sc4ccccc45)c4ccccc34)ccc12 ; c1cc(-c2c3ccccc3c(-c3ccc4sc5ccccc5c4c3)c3ccccc23)c2c(c1)oc1cc(-c3c4ccccc4c(-c4ccc5sc6ccccc6c5c4)c4ccccc34)ccc12]

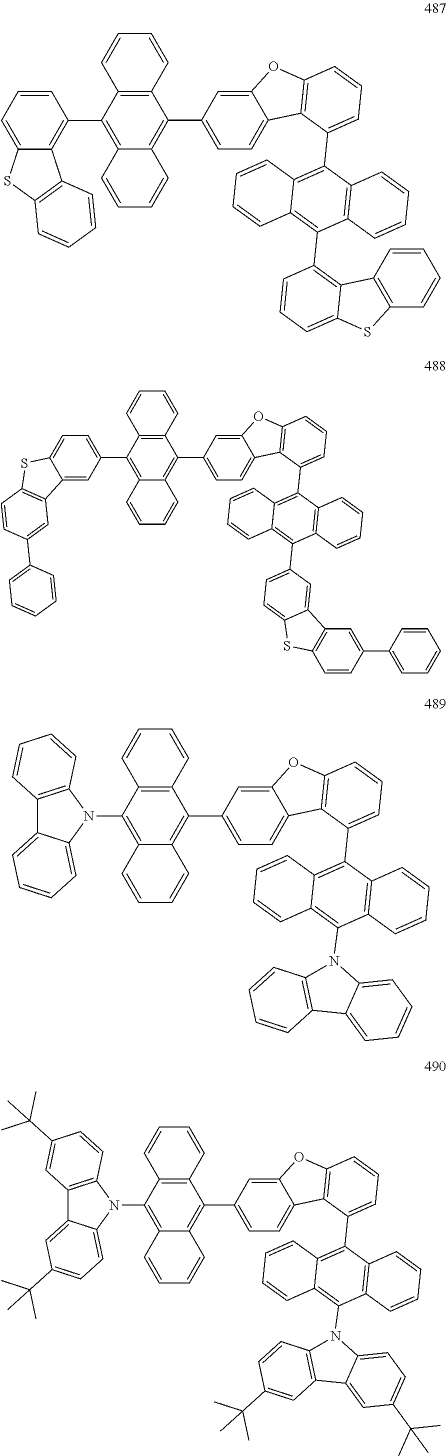

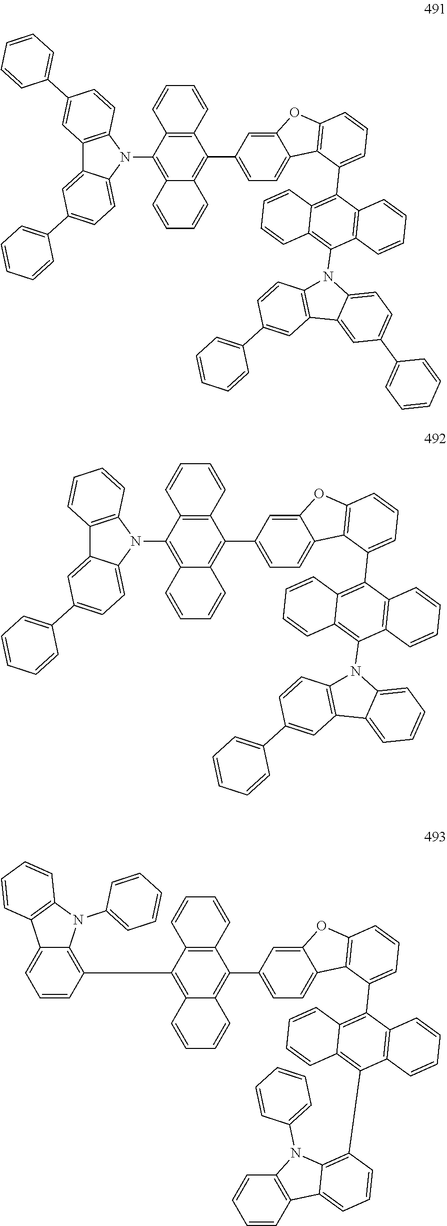 [SMILES: c1ccc(-c2ccc3c(c2)c2cc(-c4ccccc4)ccc2n3-c2c3ccccc3c(-c3ccc4c(c3)oc3cccc(-c5c6ccccc6c(-n6c7ccc(-c8ccccc8)cc7c7cc(-c8ccccc8)ccc76)c6ccccc56)c34)c3ccccc23)cc1 ; c1ccc(-c2ccc3c(c2)c2ccccc2n3-c2c3ccccc3c(-c3ccc4c(c3)oc3cccc(-c5c6ccccc6c(-n6c7ccccc7c7cc(-c8ccccc8)ccc76)c6ccccc56)c34)c3ccccc23)cc1 ; c1ccc(-n2c3ccccc3c3cccc(-c4c5ccccc5c(-c5ccc6c(c5)oc5cccc(-c7c8ccccc8c(-c8cccc9c%10ccccc%10n(-c%10ccccc%10)c89)c8ccccc78)c56)c5ccccc45)c32)cc1]

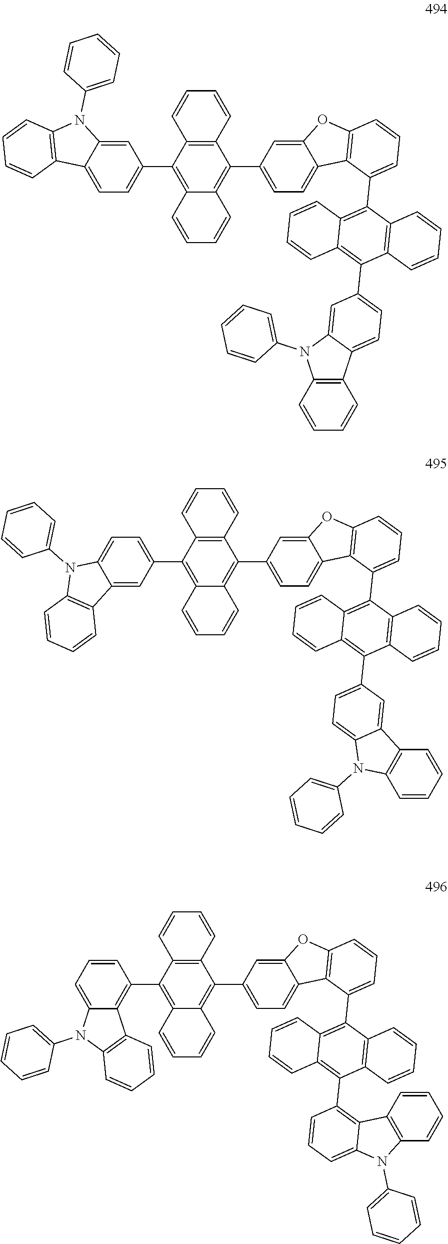 [SMILES: c1ccc(-n2c3ccccc3c3ccc(-c4c5ccccc5c(-c5ccc6c(c5)oc5cccc(-c7c8ccccc8c(-c8ccc9c%10ccccc%10n(-c%10ccccc%10)c9c8)c8ccccc78)c56)c5ccccc45)cc32)cc1 ; c1ccc(-n2c3ccccc3c3cc(-c4c5ccccc5c(-c5ccc6c(c5)oc5cccc(-c7c8ccccc8c(-c8ccc9c(c8)c8ccccc8n9-c8ccccc8)c8ccccc78)c56)c5ccccc45)ccc32)cc1 ; c1ccc(-n2c3ccccc3c3c(-c4c5ccccc5c(-c5ccc6c(c5)oc5cccc(-c7c8ccccc8c(-c8cccc9c8c8ccccc8n9-c8ccccc8)c8ccccc78)c56)c5ccccc45)cccc32)cc1]

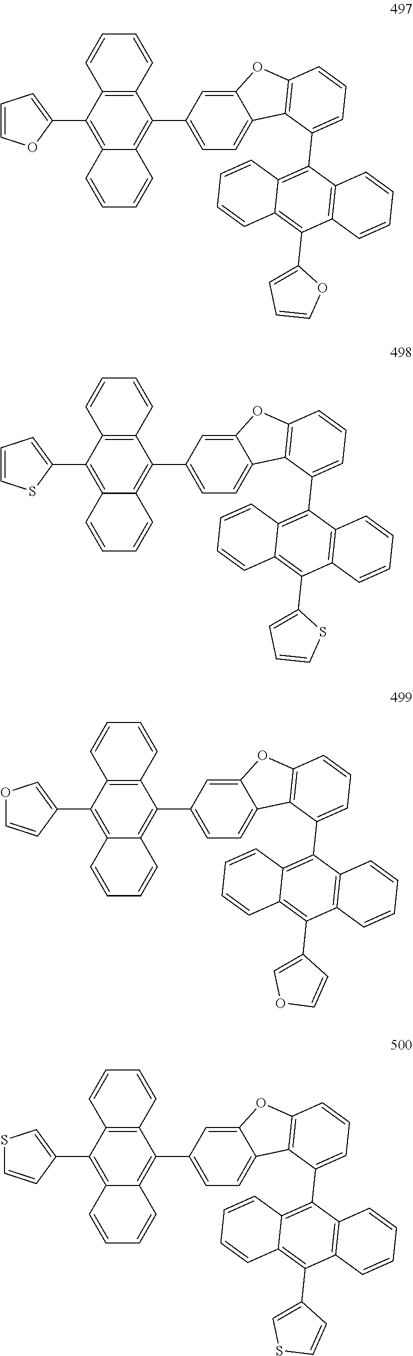 [SMILES: c1coc(-c2c3ccccc3c(-c3ccc4c(c3)oc3cccc(-c5c6ccccc6c(-c6ccco6)c6ccccc56)c34)c3ccccc23)c1 ; c1csc(-c2c3ccccc3c(-c3ccc4c(c3)oc3cccc(-c5c6ccccc6c(-c6cccs6)c6ccccc56)c34)c3ccccc23)c1 ; c1cc(-c2c3ccccc3c(-c3ccoc3)c3ccccc23)c2c(c1)oc1cc(-c3c4ccccc4c(-c4ccoc4)c4ccccc34)ccc12 ; c1cc(-c2c3ccccc3c(-c3ccsc3)c3ccccc23)c2c(c1)oc1cc(-c3c4ccccc4c(-c4ccsc4)c4ccccc34)ccc12]

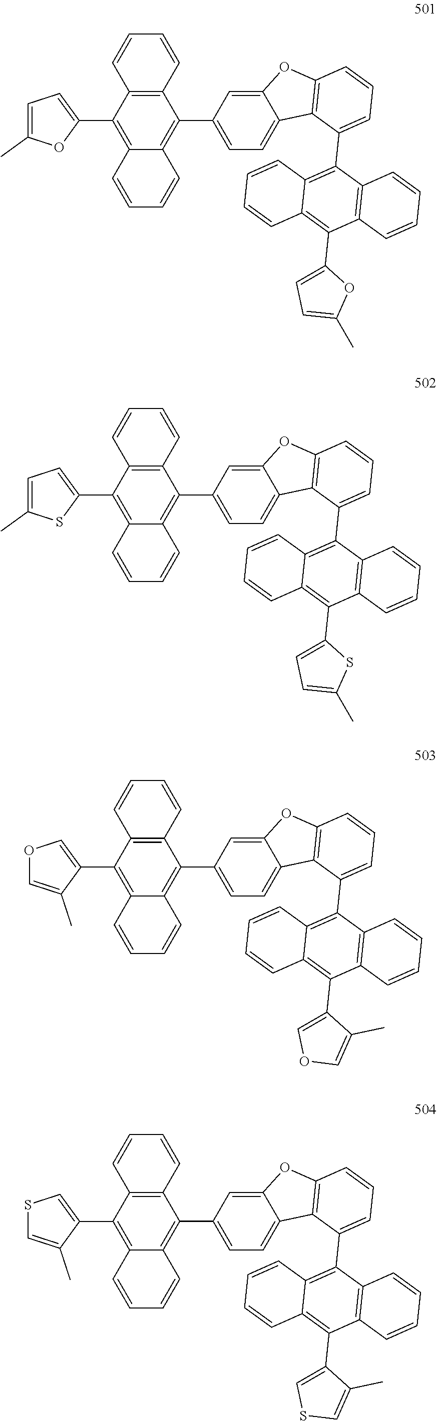 [SMILES: Cc1ccc(-c2c3ccccc3c(-c3ccc4c(c3)oc3cccc(-c5c6ccccc6c(-c6ccc(C)o6)c6ccccc56)c34)c3ccccc23)o1 ; Cc1ccc(-c2c3ccccc3c(-c3ccc4c(c3)oc3cccc(-c5c6ccccc6c(-c6ccc(C)s6)c6ccccc56)c34)c3ccccc23)s1 ; Cc1cocc1-c1c2ccccc2c(-c2ccc3c(c2)oc2cccc(-c4c5ccccc5c(-c5cocc5C)c5ccccc45)c23)c2ccccc12 ; Cc1cscc1-c1c2ccccc2c(-c2ccc3c(c2)oc2cccc(-c4c5ccccc5c(-c5cscc5C)c5ccccc45)c23)c2ccccc12]

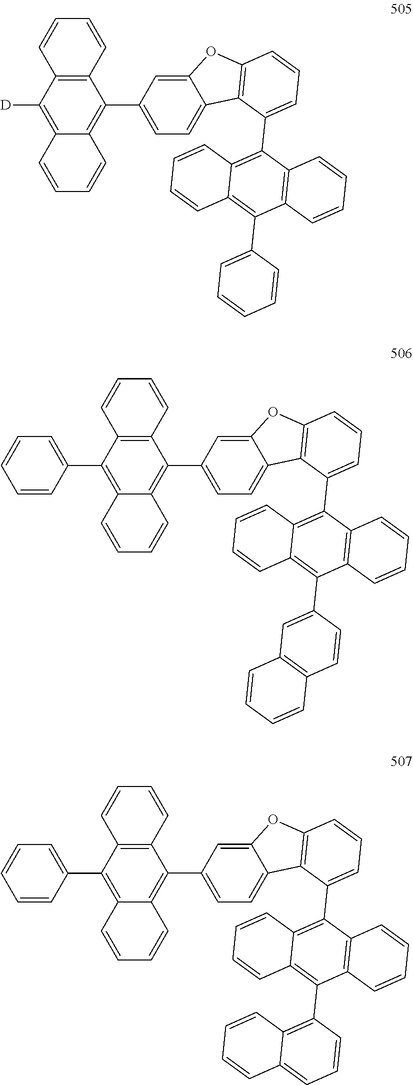 [SMILES: [2H]c1c2ccccc2c(-c2ccc3c(c2)oc2cccc(-c4c5ccccc5c(-c5ccccc5)c5ccccc45)c23)c2ccccc12 ; c1ccc(-c2c3ccccc3c(-c3ccc4c(c3)oc3cccc(-c5c6ccccc6c(-c6ccc7ccccc7c6)c6ccccc56)c34)c3ccccc23)cc1 ; c1ccc(-c2c3ccccc3c(-c3ccc4c(c3)oc3cccc(-c5c6ccccc6c(-c6cccc7ccccc67)c6ccccc56)c34)c3ccccc23)cc1]

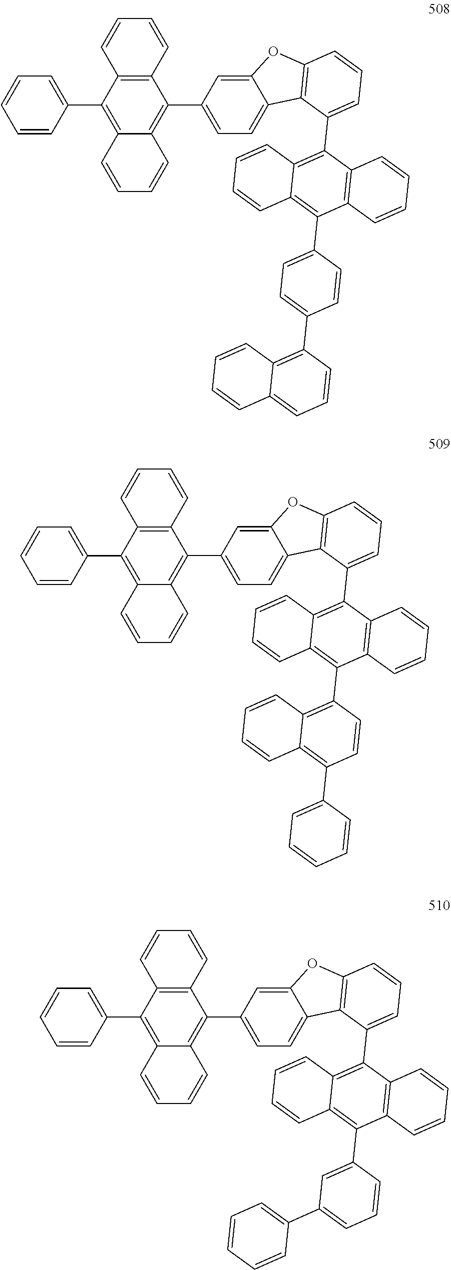 [SMILES: c1ccc(-c2c3ccccc3c(-c3ccc4c(c3)oc3cccc(-c5c6ccccc6c(-c6ccc(-c7cccc8ccccc78)cc6)c6ccccc56)c34)c3ccccc23)cc1 ; c1ccc(-c2ccc(-c3c4ccccc4c(-c4cccc5oc6cc(-c7c8ccccc8c(-c8ccccc8)c8ccccc78)ccc6c45)c4ccccc34)c3ccccc23)cc1 ; c1ccc(-c2cccc(-c3c4ccccc4c(-c4cccc5oc6cc(-c7c8ccccc8c(-c8ccccc8)c8ccccc78)ccc6c45)c4ccccc34)c2)cc1]

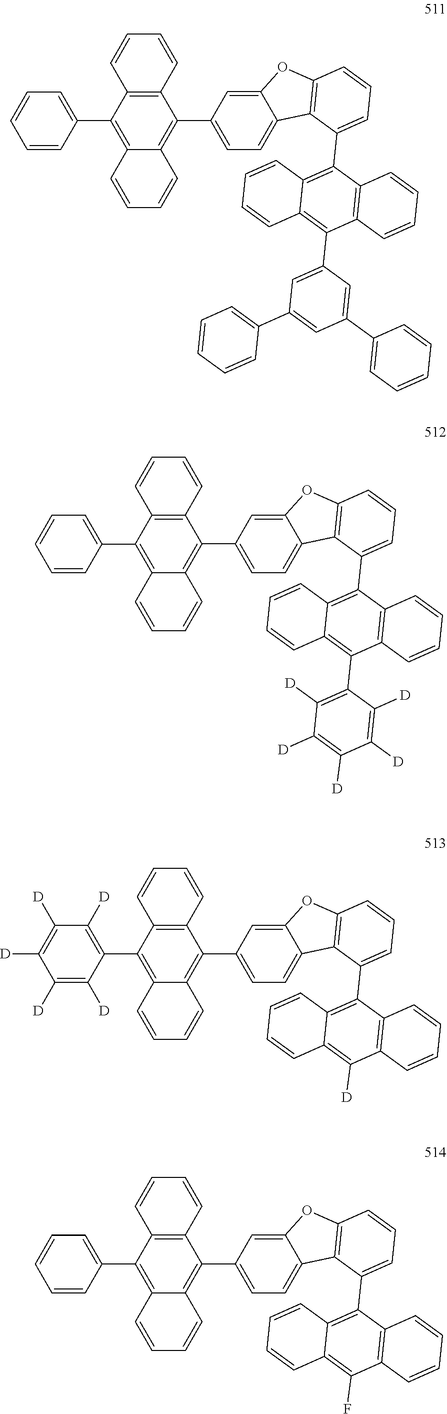 [SMILES: c1ccc(-c2cc(-c3ccccc3)cc(-c3c4ccccc4c(-c4cccc5oc6cc(-c7c8ccccc8c(-c8ccccc8)c8ccccc78)ccc6c45)c4ccccc34)c2)cc1 ; [2H]c1c([2H])c([2H])c(-c2c3ccccc3c(-c3cccc4oc5cc(-c6c7ccccc7c(-c7ccccc7)c7ccccc67)ccc5c34)c3ccccc23)c([2H])c1[2H] ; [2H]c1c([2H])c([2H])c(-c2c3ccccc3c(-c3ccc4c(c3)oc3cccc(-c5c6ccccc6c([2H])c6ccccc56)c34)c3ccccc23)c([2H])c1[2H] ; Fc1c2ccccc2c(-c2cccc3oc4cc(-c5c6ccccc6c(-c6ccccc6)c6ccccc56)ccc4c23)c2ccccc12]

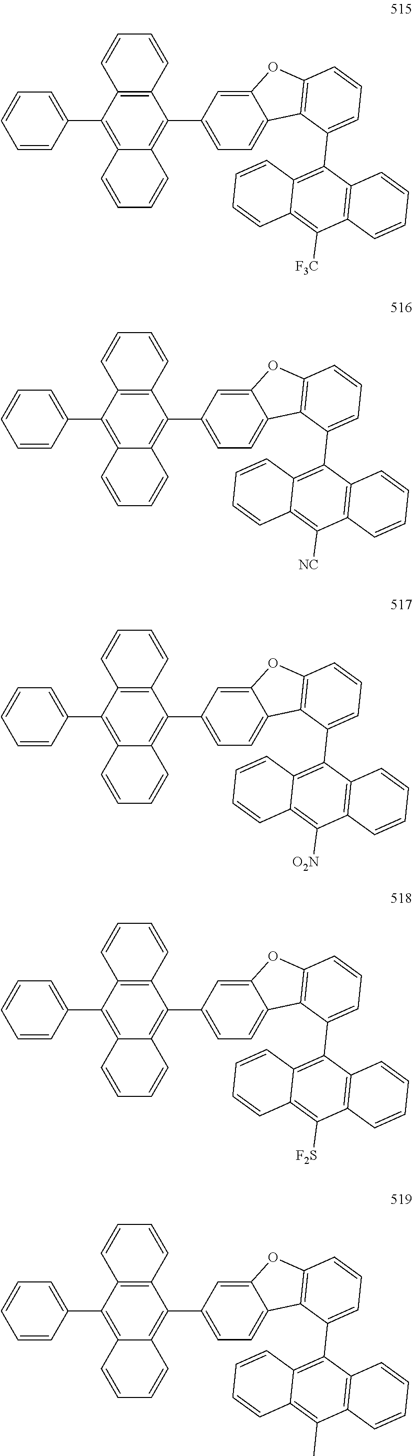

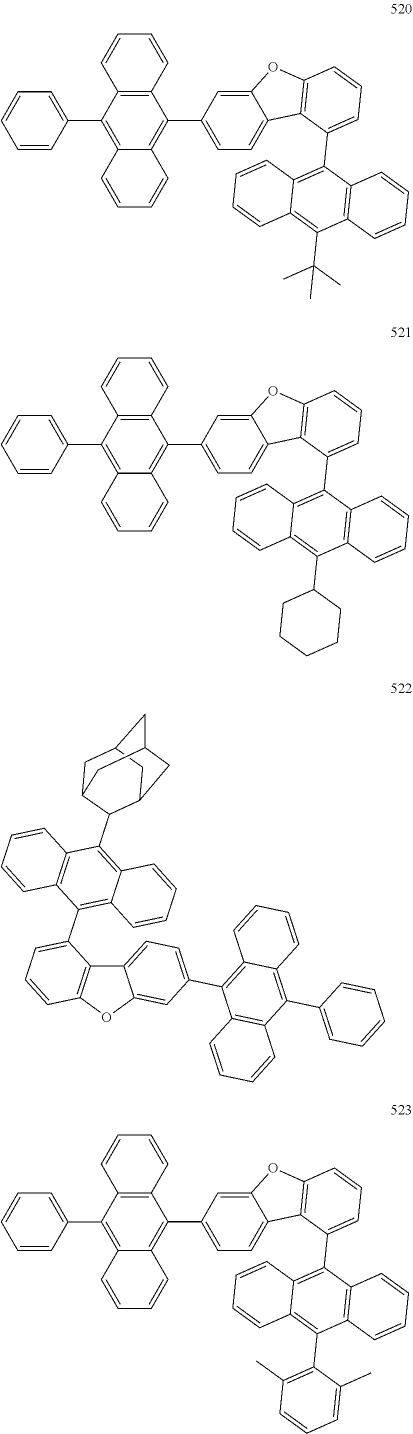 [SMILES: CC(C)(C)c1c2ccccc2c(-c2cccc3oc4cc(-c5c6ccccc6c(-c6ccccc6)c6ccccc56)ccc4c23)c2ccccc12 ; c1ccc(-c2c3ccccc3c(-c3ccc4c(c3)oc3cccc(-c5c6ccccc6c(C6CCCCC6)c6ccccc56)c34)c3ccccc23)cc1 ; c1ccc(-c2c3ccccc3c(-c3ccc4c(c3)oc3cccc(-c5c6ccccc6c(C6C7CC8CC(C7)CC6C8)c6ccccc56)c34)c3ccccc23)cc1 ; Cc1cccc(C)c1-c1c2ccccc2c(-c2cccc3oc4cc(-c5c6ccccc6c(-c6ccccc6)c6ccccc56)ccc4c23)c2ccccc12]

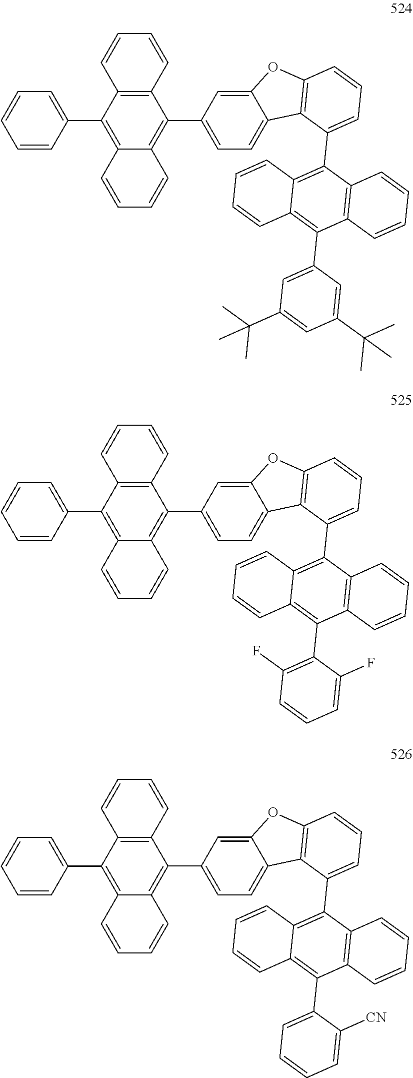 [SMILES: CC(C)(C)c1cc(-c2c3ccccc3c(-c3cccc4oc5cc(-c6c7ccccc7c(-c7ccccc7)c7ccccc67)ccc5c34)c3ccccc23)cc(C(C)(C)C)c1 ; Fc1cccc(F)c1-c1c2ccccc2c(-c2cccc3oc4cc(-c5c6ccccc6c(-c6ccccc6)c6ccccc56)ccc4c23)c2ccccc12 ; N#Cc1ccccc1-c1c2ccccc2c(-c2cccc3oc4cc(-c5c6ccccc6c(-c6ccccc6)c6ccccc56)ccc4c23)c2ccccc12]

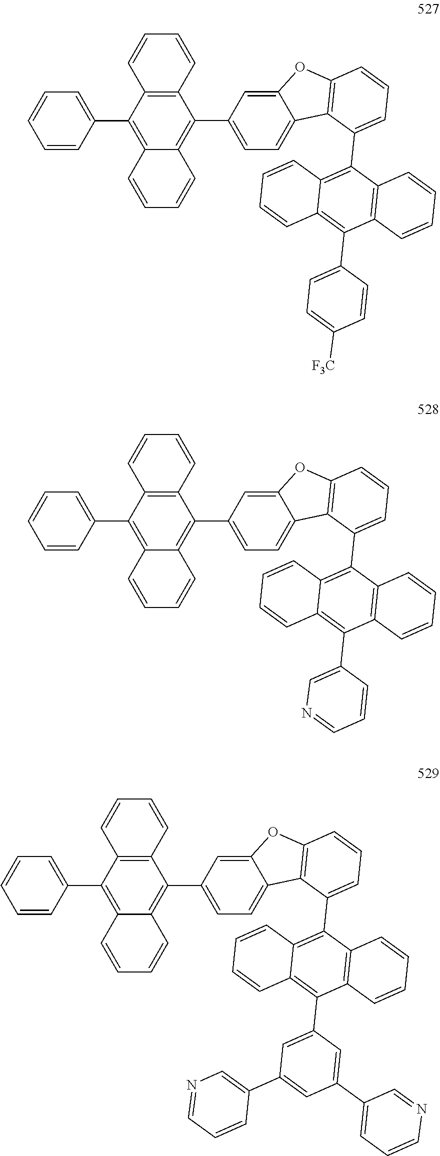 [SMILES: FC(F)(F)c1ccc(-c2c3ccccc3c(-c3cccc4oc5cc(-c6c7ccccc7c(-c7ccccc7)c7ccccc67)ccc5c34)c3ccccc23)cc1 ; c1ccc(-c2c3ccccc3c(-c3ccc4c(c3)oc3cccc(-c5c6ccccc6c(-c6cccnc6)c6ccccc56)c34)c3ccccc23)cc1 ; c1ccc(-c2c3ccccc3c(-c3ccc4c(c3)oc3cccc(-c5c6ccccc6c(-c6cc(-c7cccnc7)cc(-c7cccnc7)c6)c6ccccc56)c34)c3ccccc23)cc1]

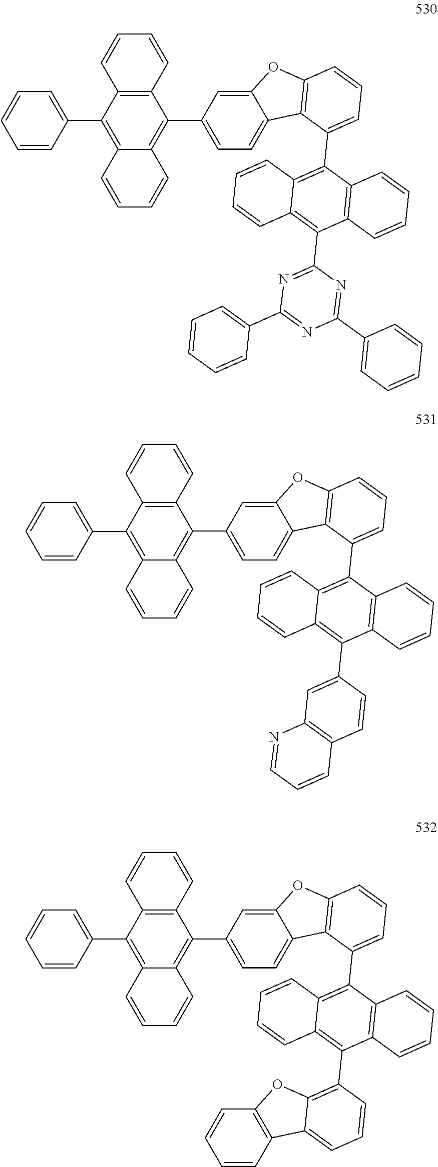 [SMILES: c1ccc(-c2nc(-c3ccccc3)nc(-c3c4ccccc4c(-c4cccc5oc6cc(-c7c8ccccc8c(-c8ccccc8)c8ccccc78)ccc6c45)c4ccccc34)n2)cc1 ; c1ccc(-c2c3ccccc3c(-c3ccc4c(c3)oc3cccc(-c5c6ccccc6c(-c6ccc7cccnc7c6)c6ccccc56)c34)c3ccccc23)cc1 ; c1ccc(-c2c3ccccc3c(-c3ccc4c(c3)oc3cccc(-c5c6ccccc6c(-c6cccc7c6oc6ccccc67)c6ccccc56)c34)c3ccccc23)cc1]

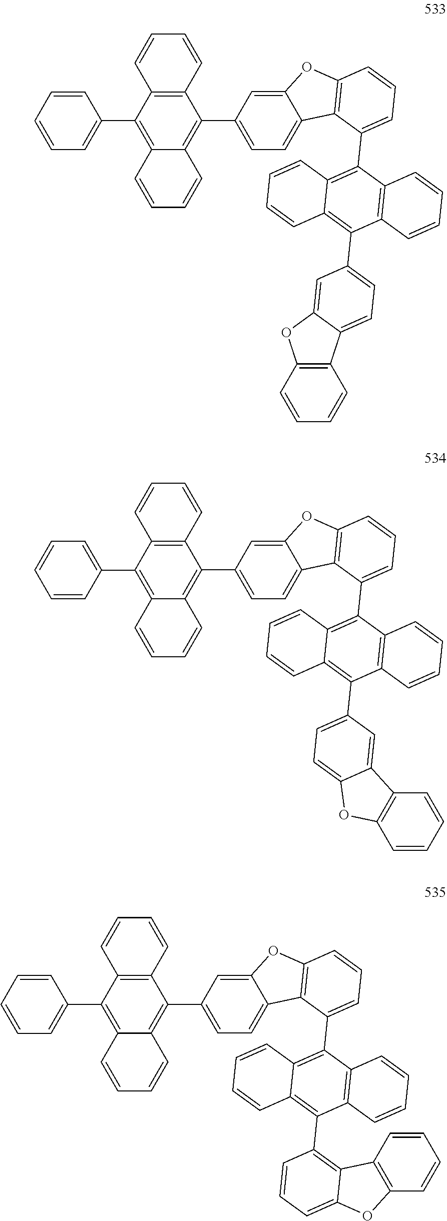 [SMILES: c1ccc(-c2c3ccccc3c(-c3ccc4c(c3)oc3cccc(-c5c6ccccc6c(-c6ccc7c(c6)oc6ccccc67)c6ccccc56)c34)c3ccccc23)cc1 ; c1ccc(-c2c3ccccc3c(-c3ccc4c(c3)oc3cccc(-c5c6ccccc6c(-c6ccc7oc8ccccc8c7c6)c6ccccc56)c34)c3ccccc23)cc1 ; c1ccc(-c2c3ccccc3c(-c3ccc4c(c3)oc3cccc(-c5c6ccccc6c(-c6cccc7oc8ccccc8c67)c6ccccc56)c34)c3ccccc23)cc1]

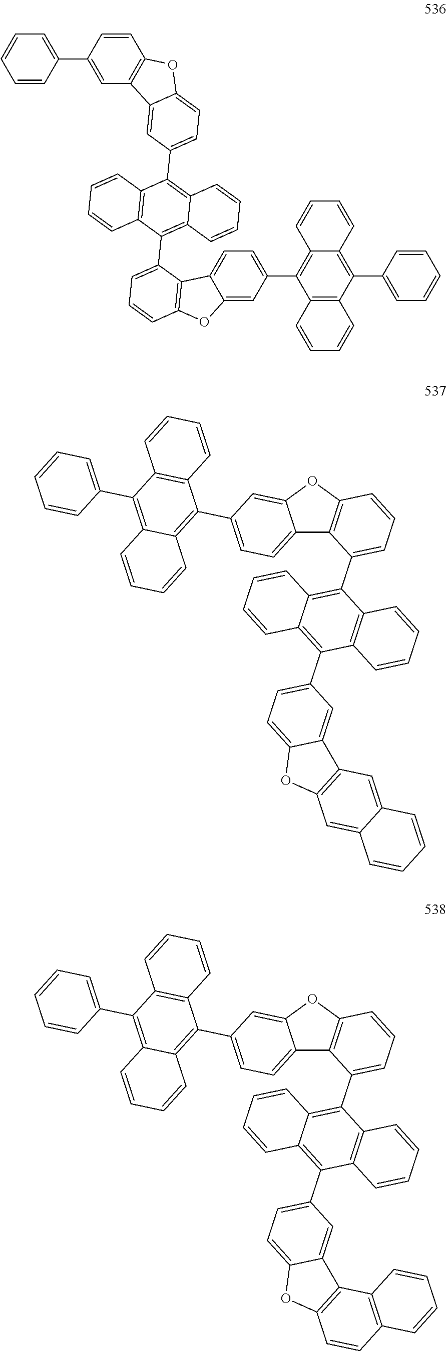 [SMILES: c1ccc(-c2ccc3oc4ccc(-c5c6ccccc6c(-c6cccc7oc8cc(-c9c%10ccccc%10c(-c%10ccccc%10)c%10ccccc9%10)ccc8c67)c6ccccc56)cc4c3c2)cc1 ; c1ccc(-c2c3ccccc3c(-c3ccc4c(c3)oc3cccc(-c5c6ccccc6c(-c6ccc7oc8cc9ccccc9cc8c7c6)c6ccccc56)c34)c3ccccc23)cc1 ; c1ccc(-c2c3ccccc3c(-c3ccc4c(c3)oc3cccc(-c5c6ccccc6c(-c6ccc7oc8ccc9ccccc9c8c7c6)c6ccccc56)c34)c3ccccc23)cc1]

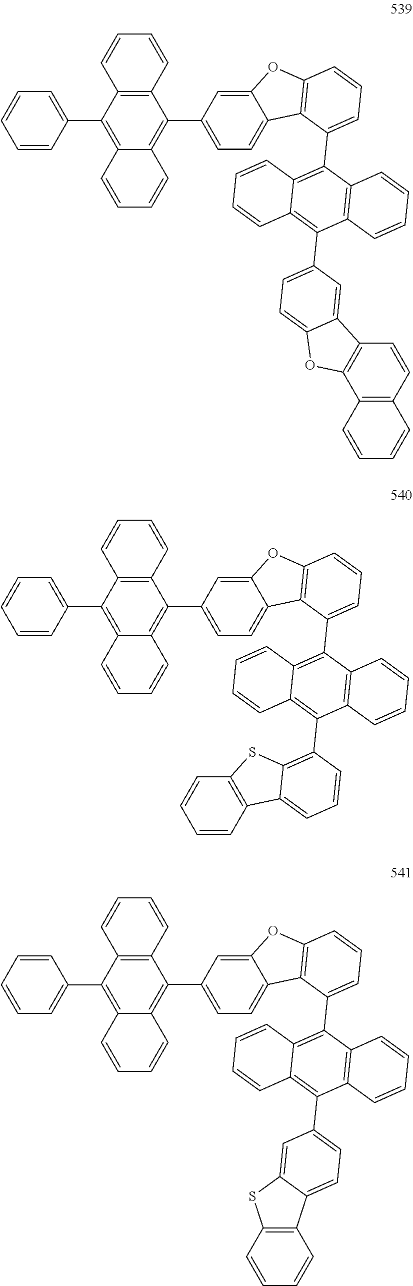 [SMILES: c1ccc(-c2c3ccccc3c(-c3ccc4c(c3)oc3cccc(-c5c6ccccc6c(-c6ccc7oc8c9ccccc9ccc8c7c6)c6ccccc56)c34)c3ccccc23)cc1 ; c1ccc(-c2c3ccccc3c(-c3ccc4c(c3)oc3cccc(-c5c6ccccc6c(-c6cccc7c6sc6ccccc67)c6ccccc56)c34)c3ccccc23)cc1 ; c1ccc(-c2c3ccccc3c(-c3ccc4c(c3)oc3cccc(-c5c6ccccc6c(-c6ccc7c(c6)sc6ccccc67)c6ccccc56)c34)c3ccccc23)cc1]

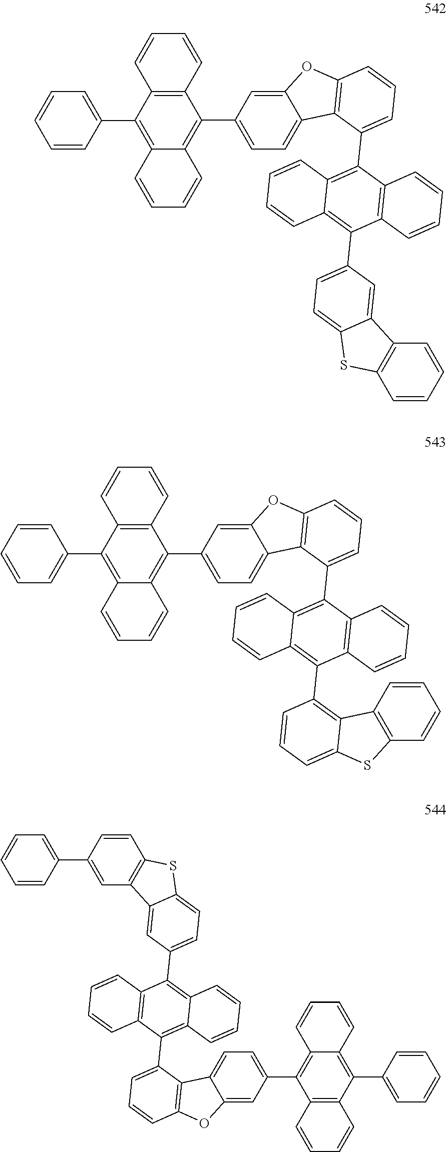 [SMILES: c1ccc(-c2c3ccccc3c(-c3ccc4c(c3)oc3cccc(-c5c6ccccc6c(-c6ccc7sc8ccccc8c7c6)c6ccccc56)c34)c3ccccc23)cc1 ; c1ccc(-c2c3ccccc3c(-c3ccc4c(c3)oc3cccc(-c5c6ccccc6c(-c6cccc7sc8ccccc8c67)c6ccccc56)c34)c3ccccc23)cc1 ; c1ccc(-c2ccc3sc4ccc(-c5c6ccccc6c(-c6cccc7oc8cc(-c9c%10ccccc%10c(-c%10ccccc%10)c%10ccccc9%10)ccc8c67)c6ccccc56)cc4c3c2)cc1]

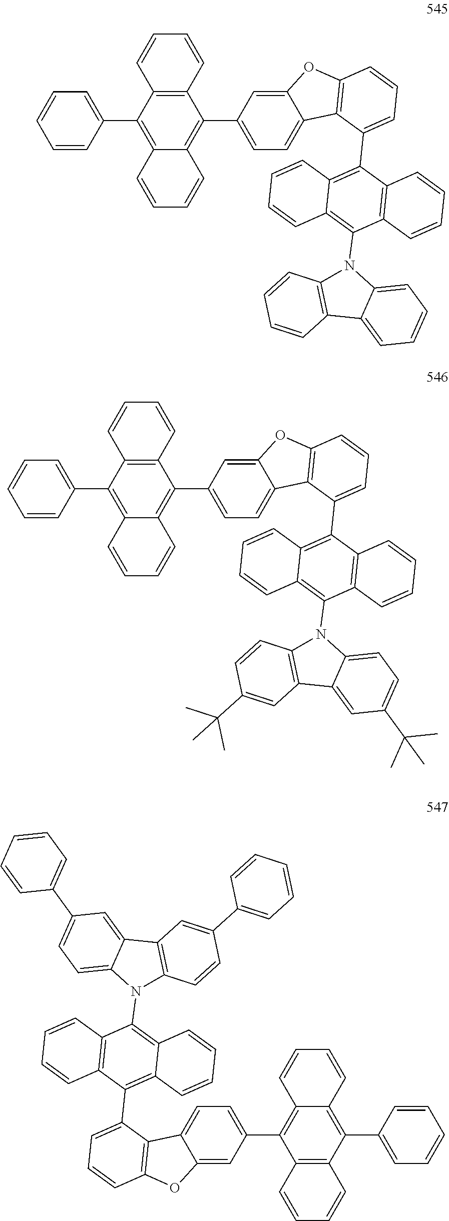 [SMILES: c1ccc(-c2c3ccccc3c(-c3ccc4c(c3)oc3cccc(-c5c6ccccc6c(-n6c7ccccc7c7ccccc76)c6ccccc56)c34)c3ccccc23)cc1 ; CC(C)(C)c1ccc2c(c1)c1cc(C(C)(C)C)ccc1n2-c1c2ccccc2c(-c2cccc3oc4cc(-c5c6ccccc6c(-c6ccccc6)c6ccccc56)ccc4c23)c2ccccc12 ; c1ccc(-c2ccc3c(c2)c2cc(-c4ccccc4)ccc2n3-c2c3ccccc3c(-c3cccc4oc5cc(-c6c7ccccc7c(-c7ccccc7)c7ccccc67)ccc5c34)c3ccccc23)cc1]

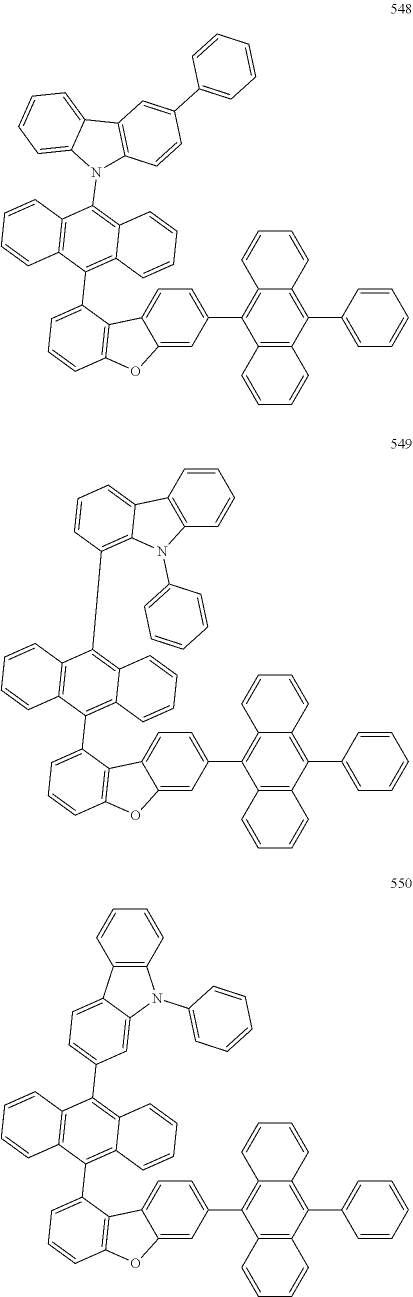 [SMILES: c1ccc(-c2ccc3c(c2)c2ccccc2n3-c2c3ccccc3c(-c3cccc4oc5cc(-c6c7ccccc7c(-c7ccccc7)c7ccccc67)ccc5c34)c3ccccc23)cc1 ; c1ccc(-c2c3ccccc3c(-c3ccc4c(c3)oc3cccc(-c5c6ccccc6c(-c6cccc7c8ccccc8n(-c8ccccc8)c67)c6ccccc56)c34)c3ccccc23)cc1 ; c1ccc(-c2c3ccccc3c(-c3ccc4c(c3)oc3cccc(-c5c6ccccc6c(-c6ccc7c8ccccc8n(-c8ccccc8)c7c6)c6ccccc56)c34)c3ccccc23)cc1]

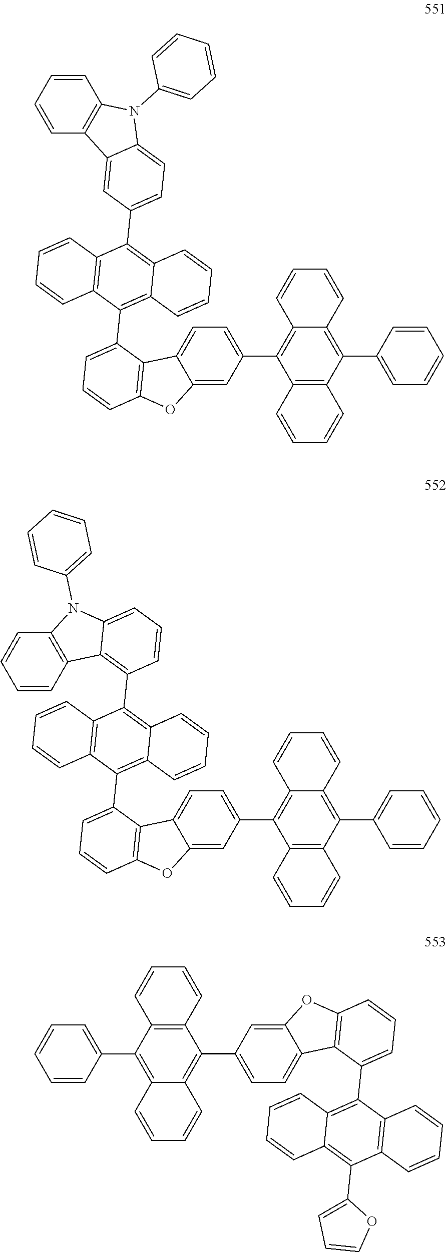 [SMILES: c1ccc(-c2c3ccccc3c(-c3ccc4c(c3)oc3cccc(-c5c6ccccc6c(-c6ccc7c(c6)c6ccccc6n7-c6ccccc6)c6ccccc56)c34)c3ccccc23)cc1 ; c1ccc(-c2c3ccccc3c(-c3ccc4c(c3)oc3cccc(-c5c6ccccc6c(-c6cccc7c6c6ccccc6n7-c6ccccc6)c6ccccc56)c34)c3ccccc23)cc1 ; c1ccc(-c2c3ccccc3c(-c3ccc4c(c3)oc3cccc(-c5c6ccccc6c(-c6ccco6)c6ccccc56)c34)c3ccccc23)cc1]

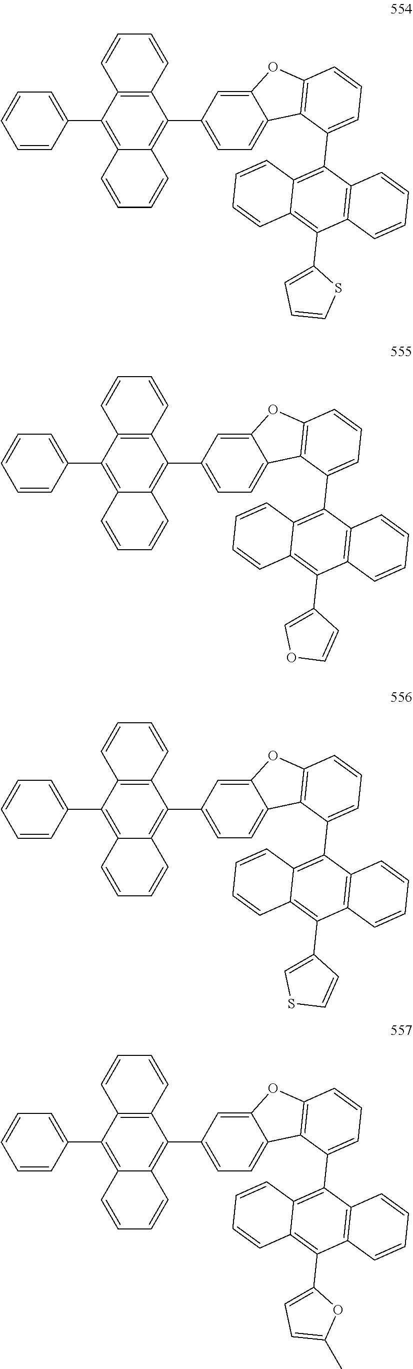 [SMILES: c1ccc(-c2c3ccccc3c(-c3ccc4c(c3)oc3cccc(-c5c6ccccc6c(-c6cccs6)c6ccccc56)c34)c3ccccc23)cc1 ; c1ccc(-c2c3ccccc3c(-c3ccc4c(c3)oc3cccc(-c5c6ccccc6c(-c6ccoc6)c6ccccc56)c34)c3ccccc23)cc1 ; c1ccc(-c2c3ccccc3c(-c3ccc4c(c3)oc3cccc(-c5c6ccccc6c(-c6ccsc6)c6ccccc56)c34)c3ccccc23)cc1 ; Cc1ccc(-c2c3ccccc3c(-c3cccc4oc5cc(-c6c7ccccc7c(-c7ccccc7)c7ccccc67)ccc5c34)c3ccccc23)o1]

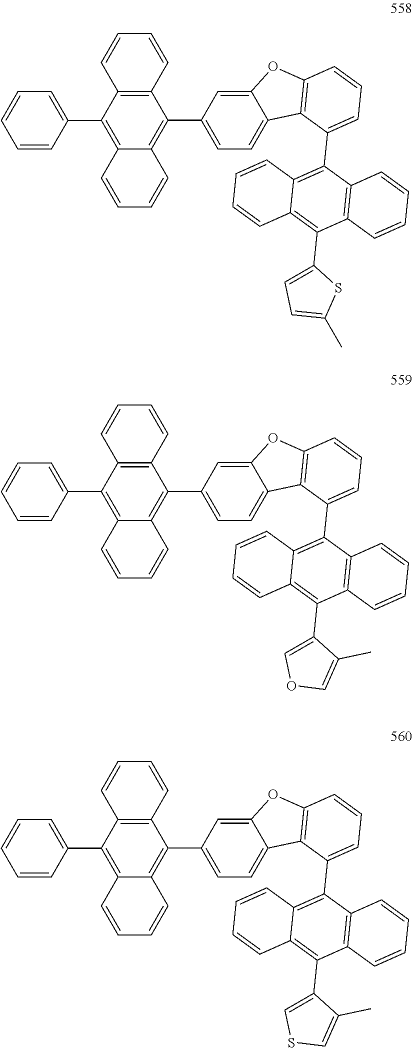 [SMILES: Cc1ccc(-c2c3ccccc3c(-c3cccc4oc5cc(-c6c7ccccc7c(-c7ccccc7)c7ccccc67)ccc5c34)c3ccccc23)s1 ; Cc1cocc1-c1c2ccccc2c(-c2cccc3oc4cc(-c5c6ccccc6c(-c6ccccc6)c6ccccc56)ccc4c23)c2ccccc12 ; Cc1cscc1-c1c2ccccc2c(-c2cccc3oc4cc(-c5c6ccccc6c(-c6ccccc6)c6ccccc56)ccc4c23)c2ccccc12]

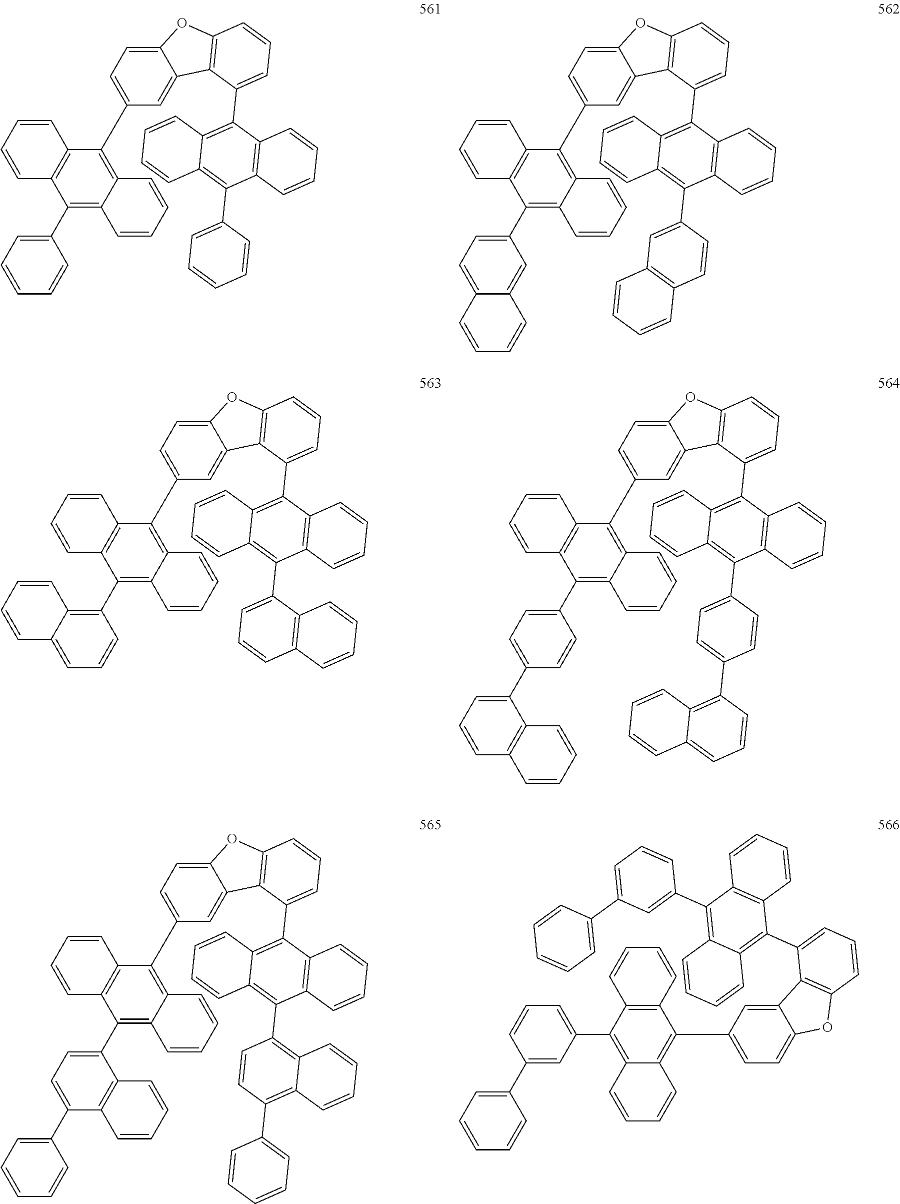

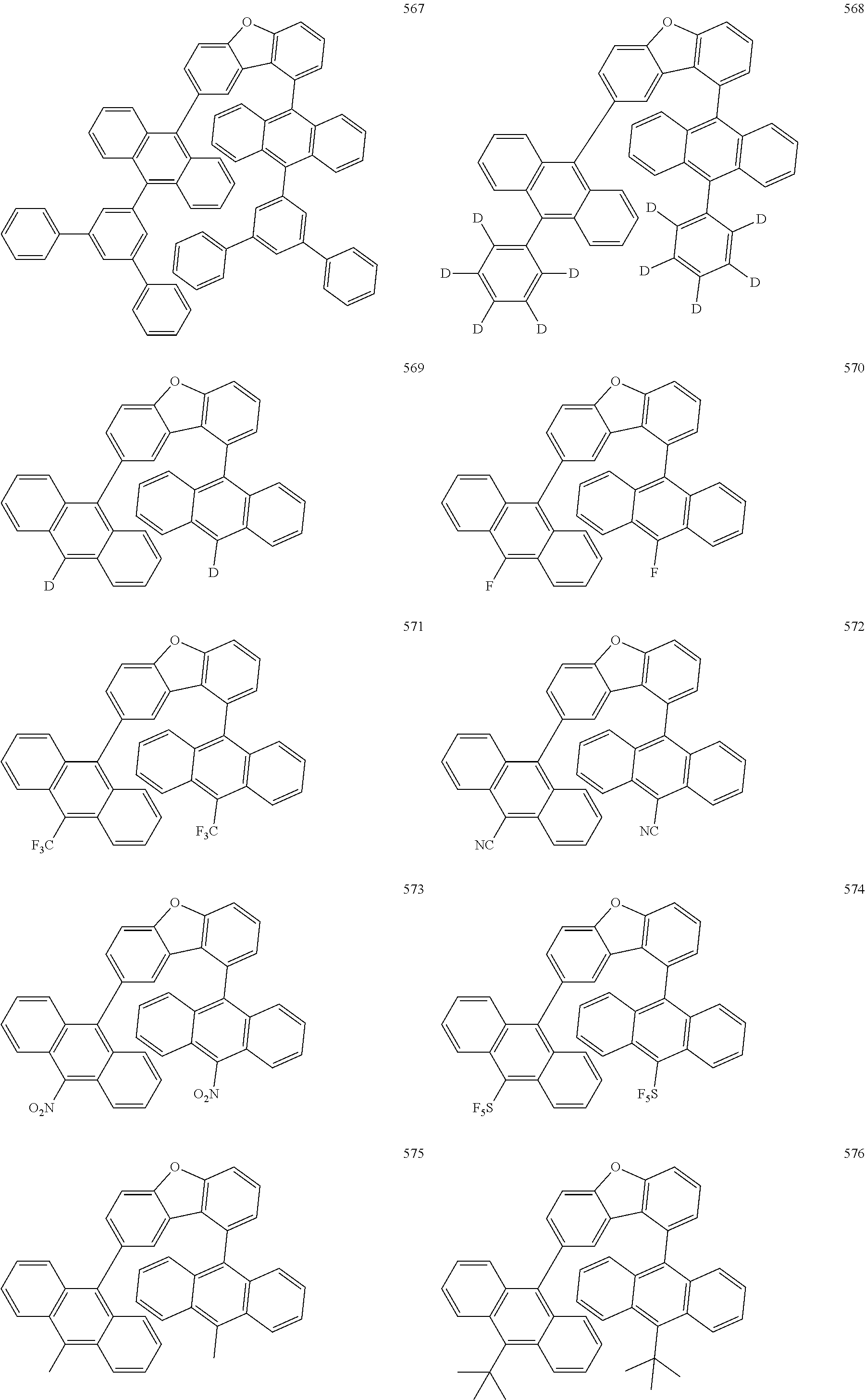

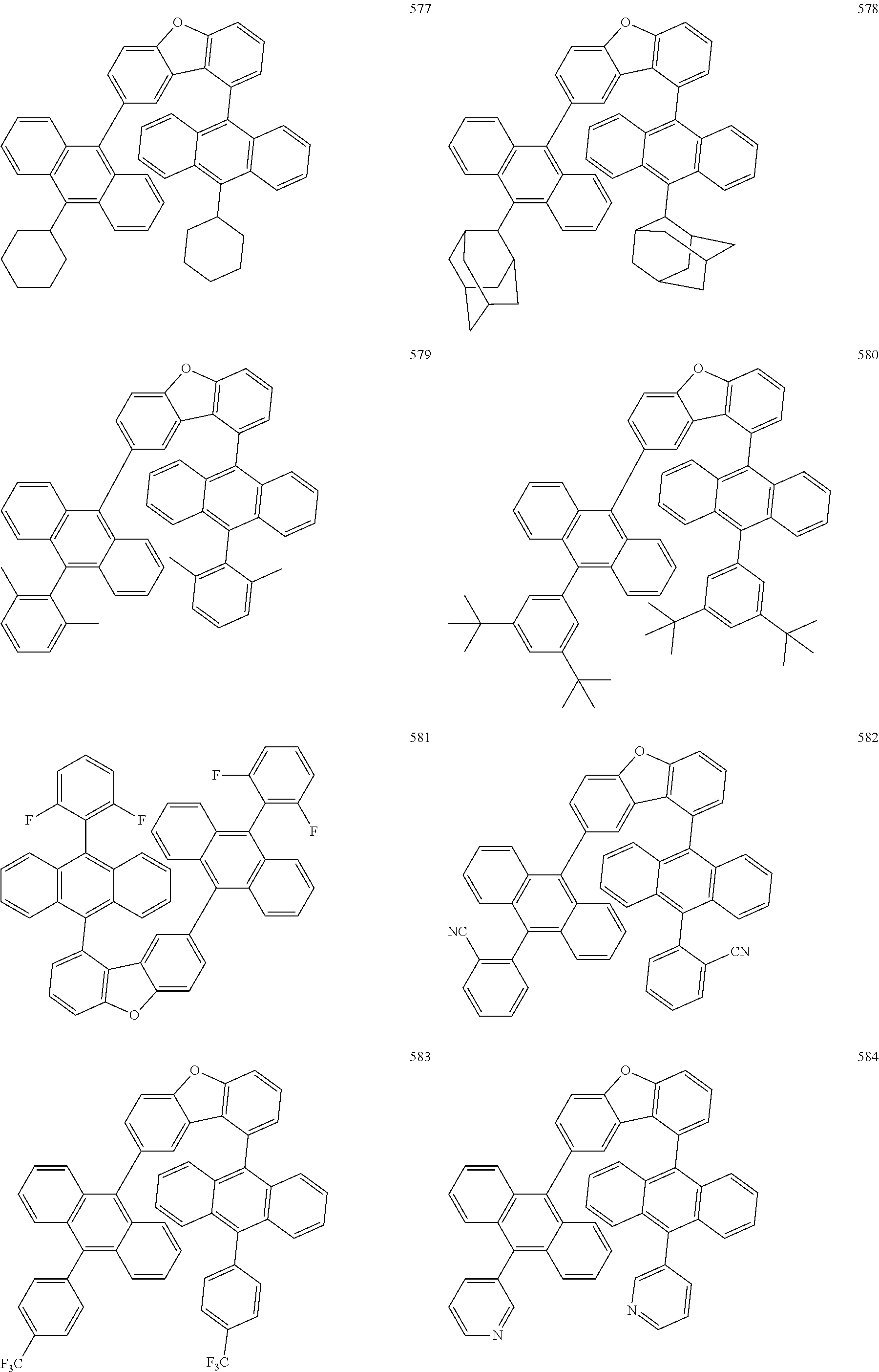

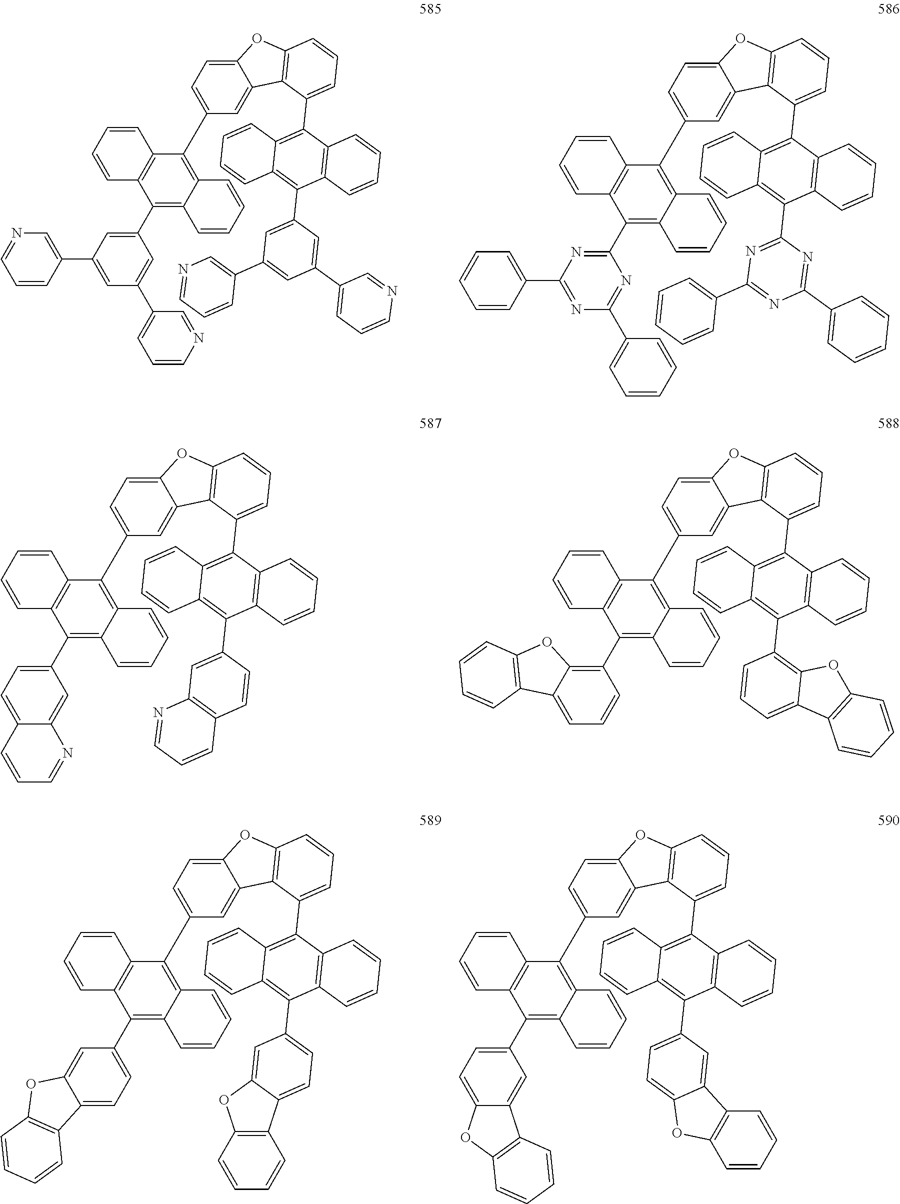

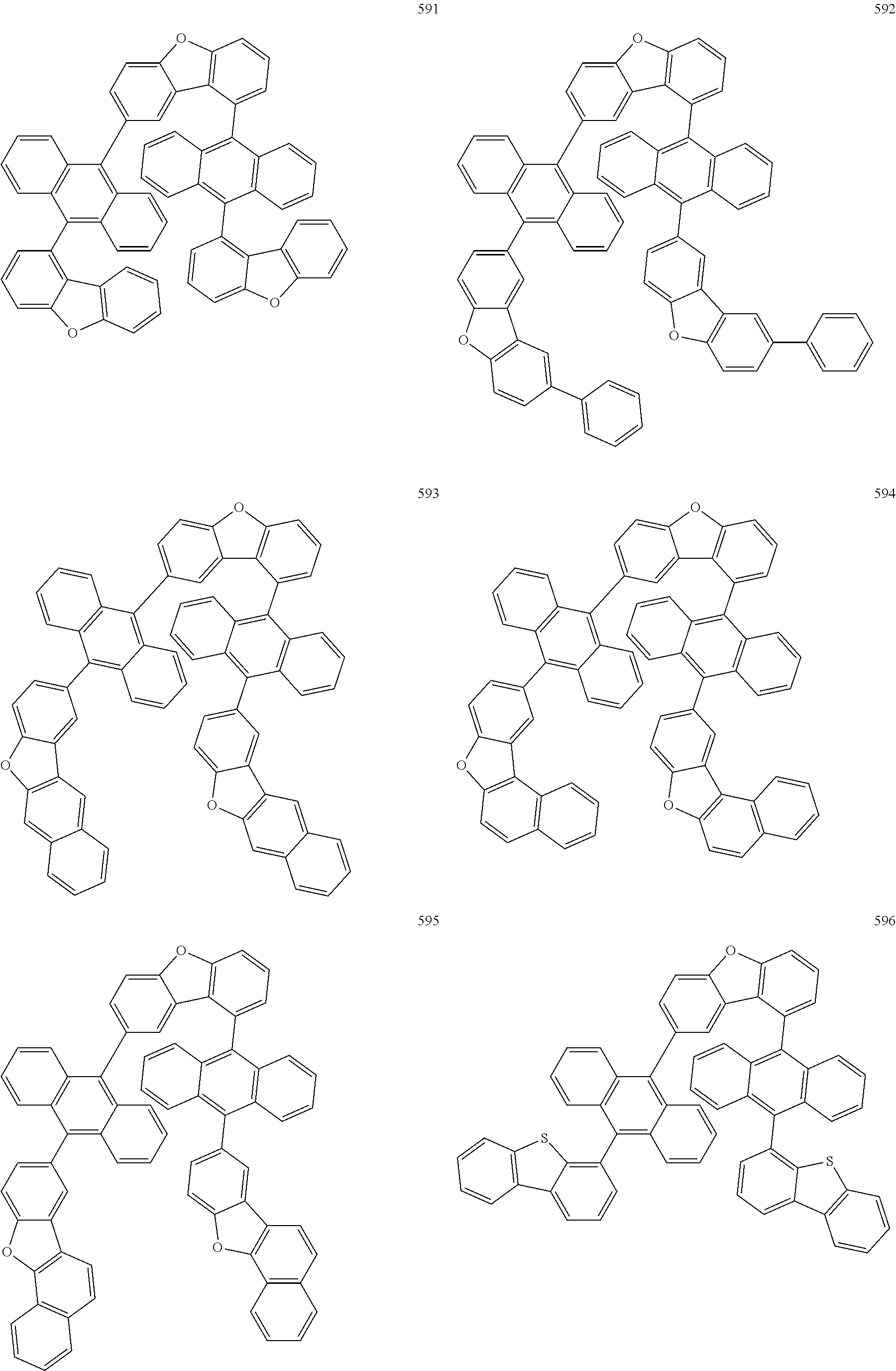

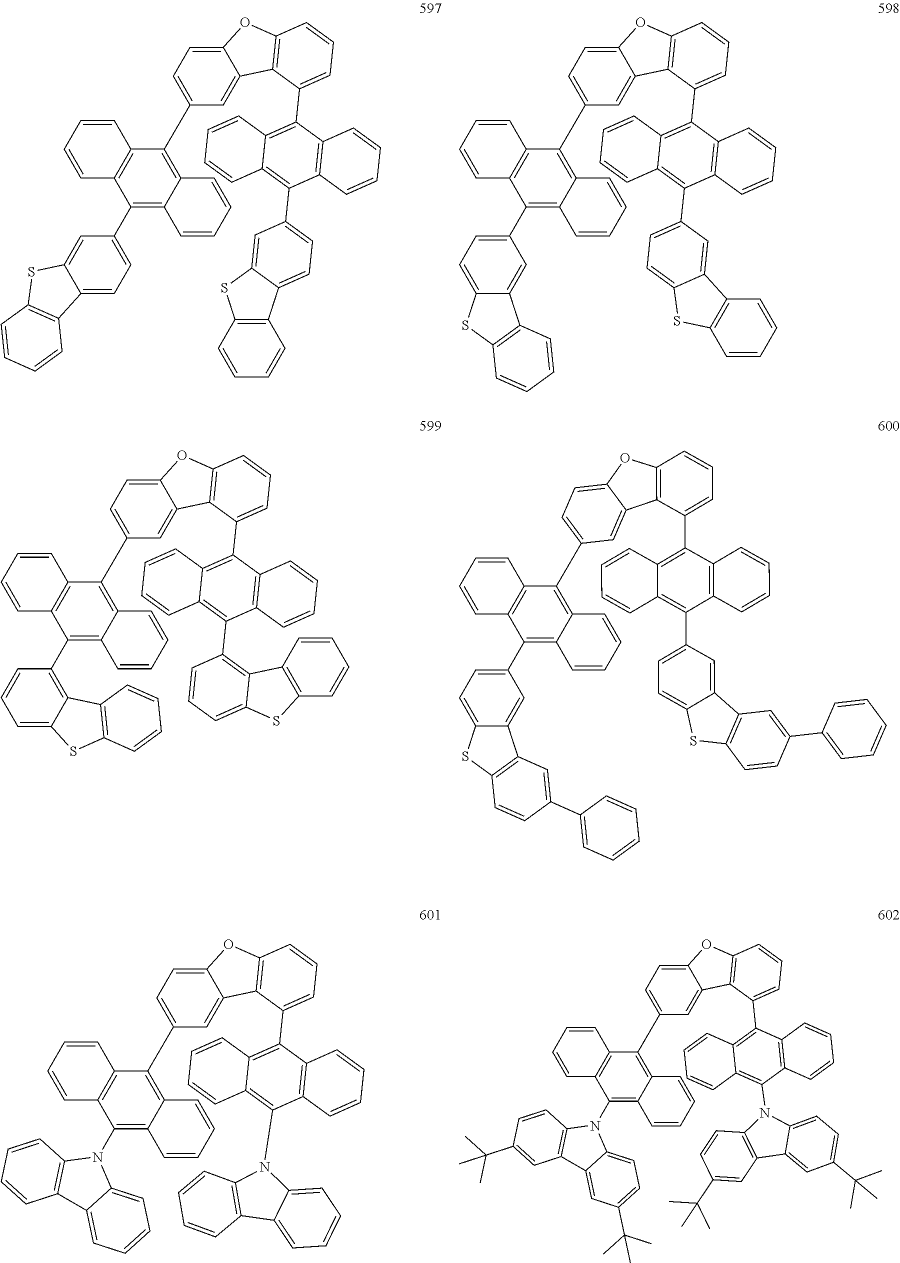

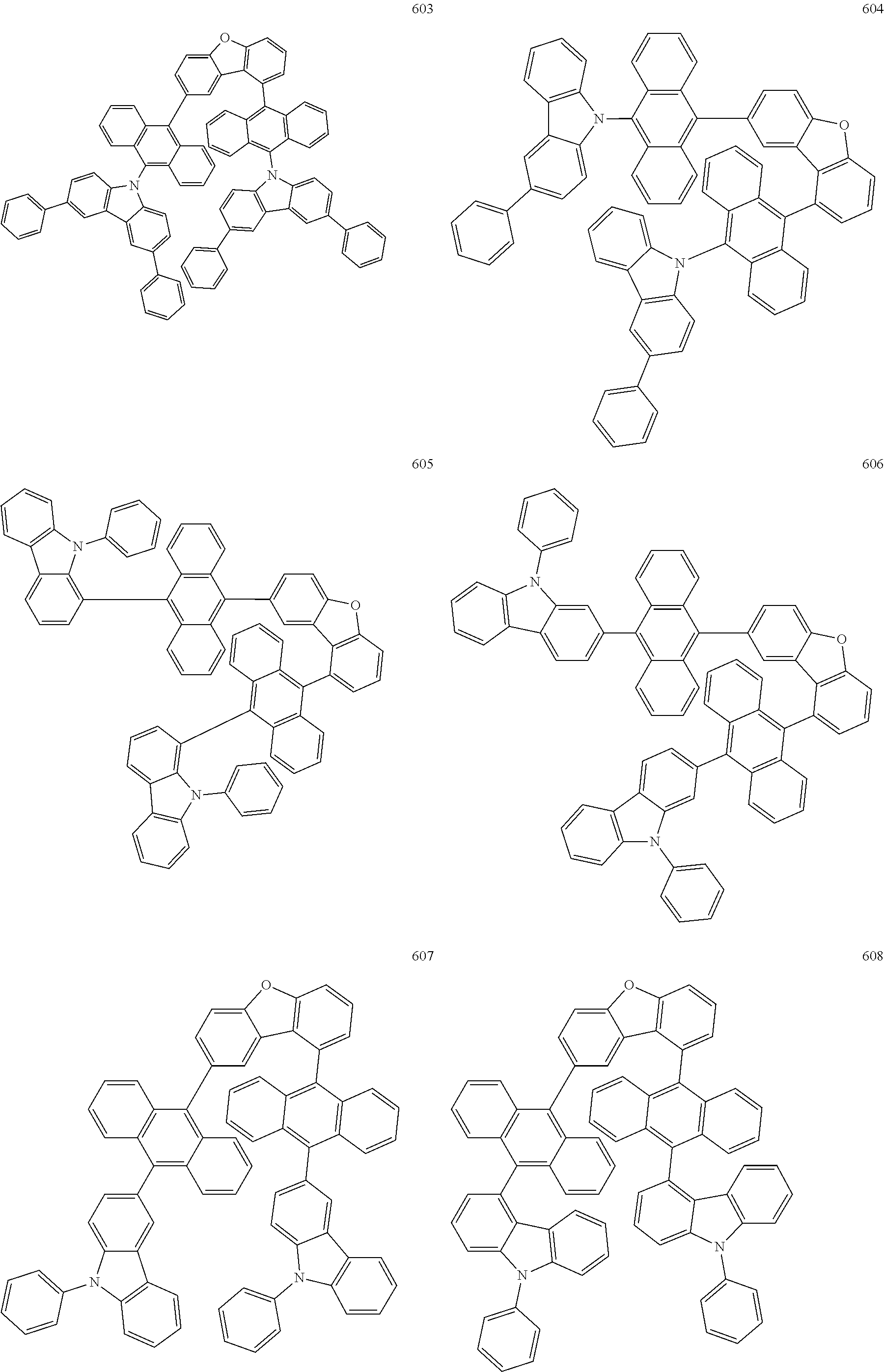

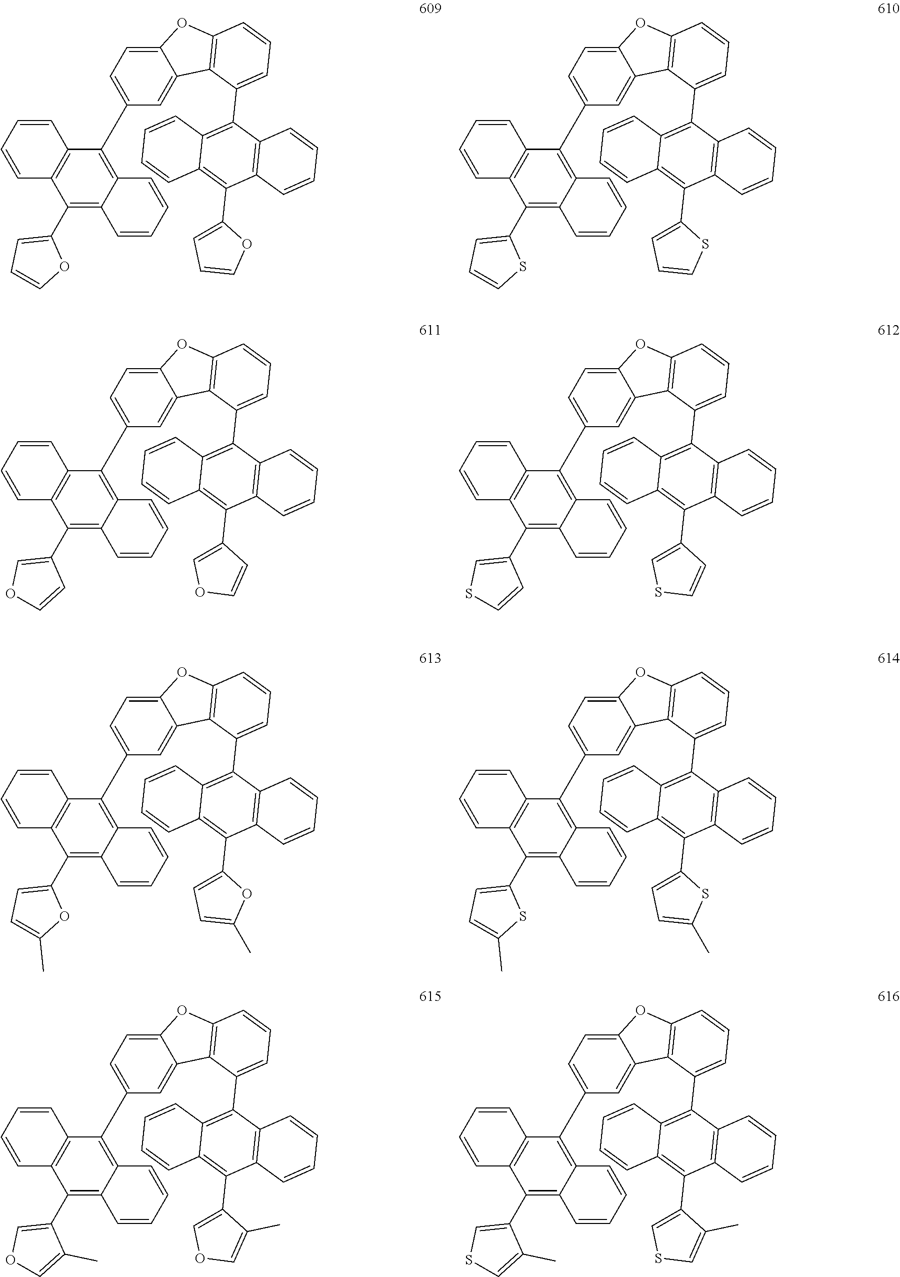

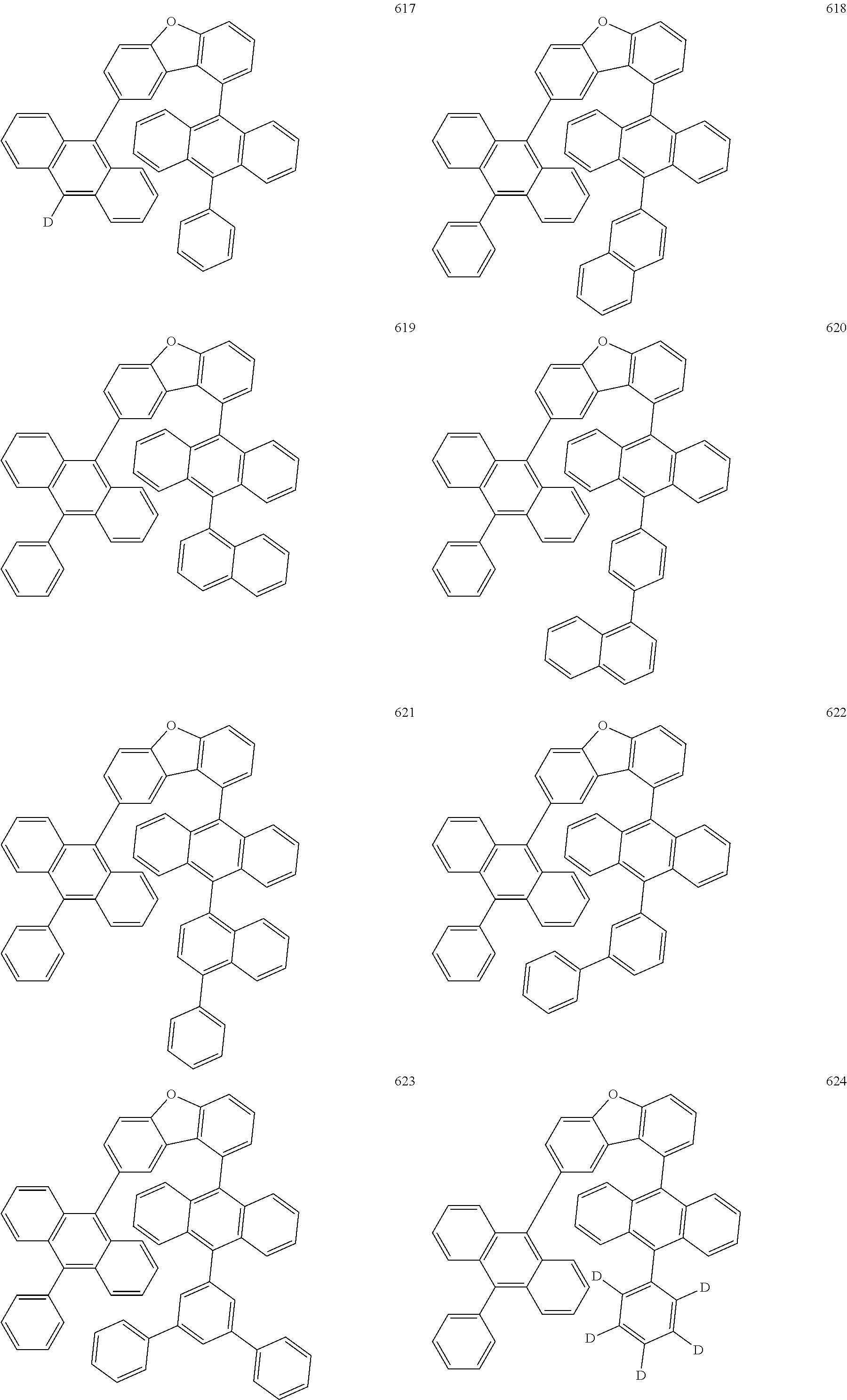

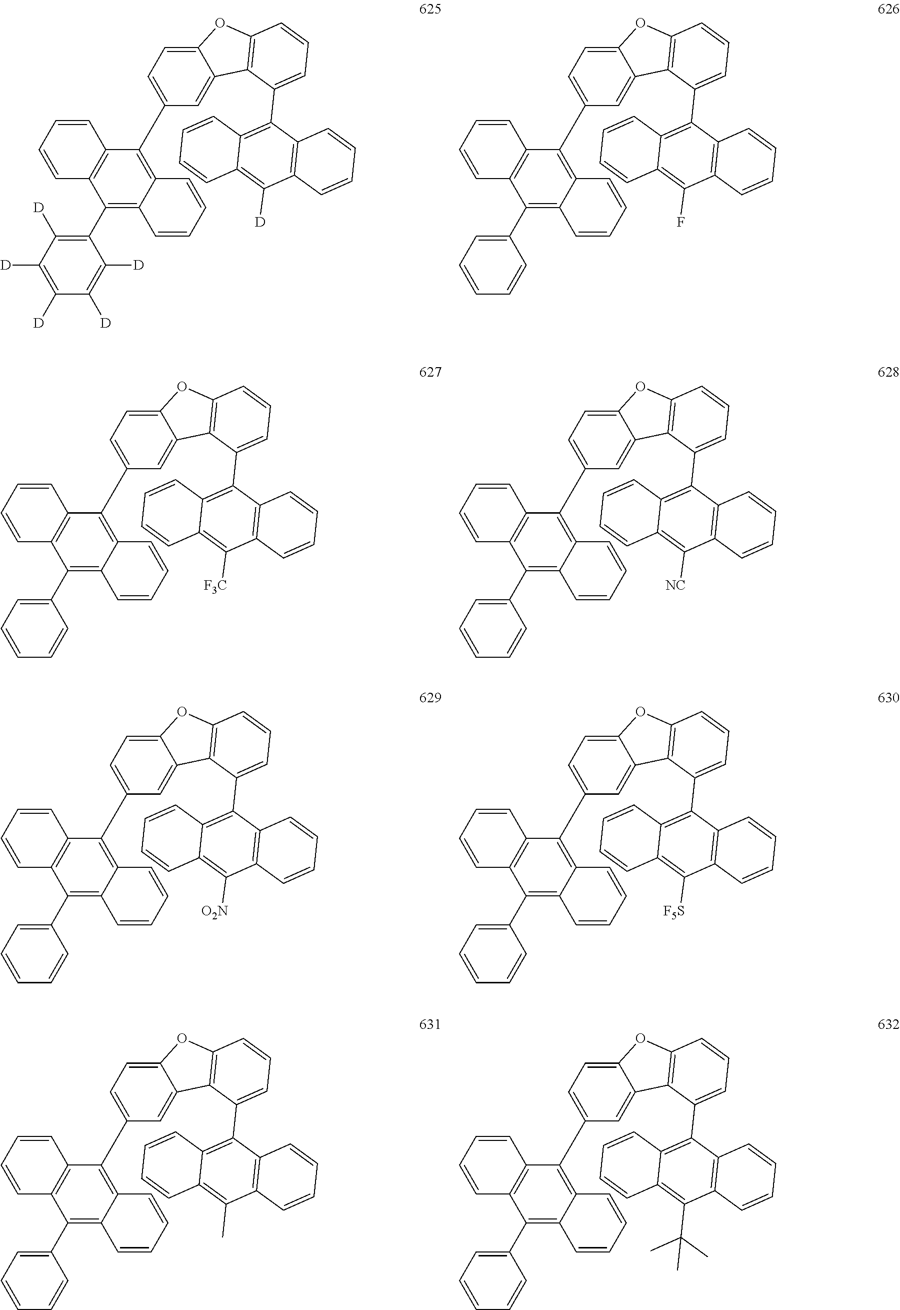

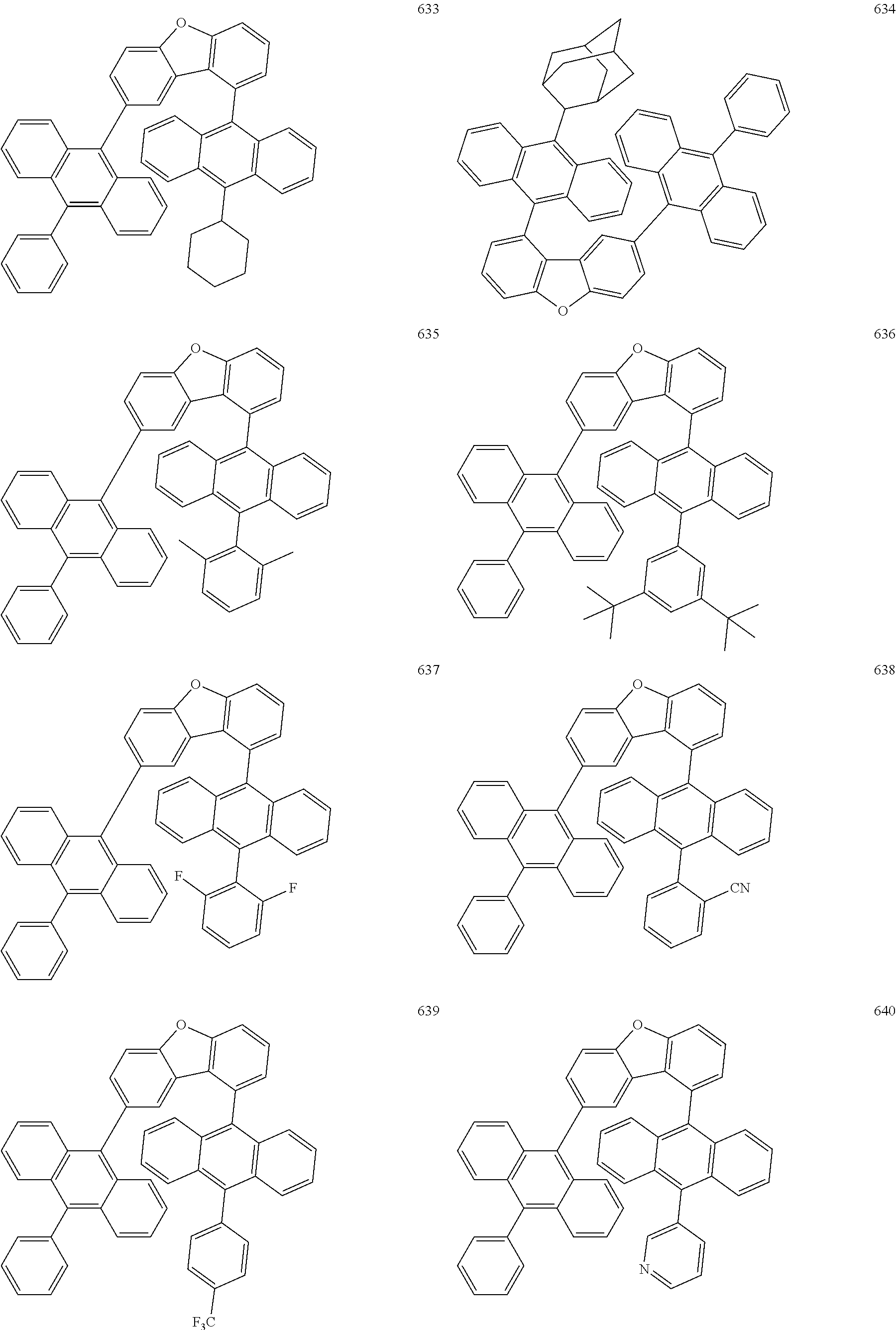

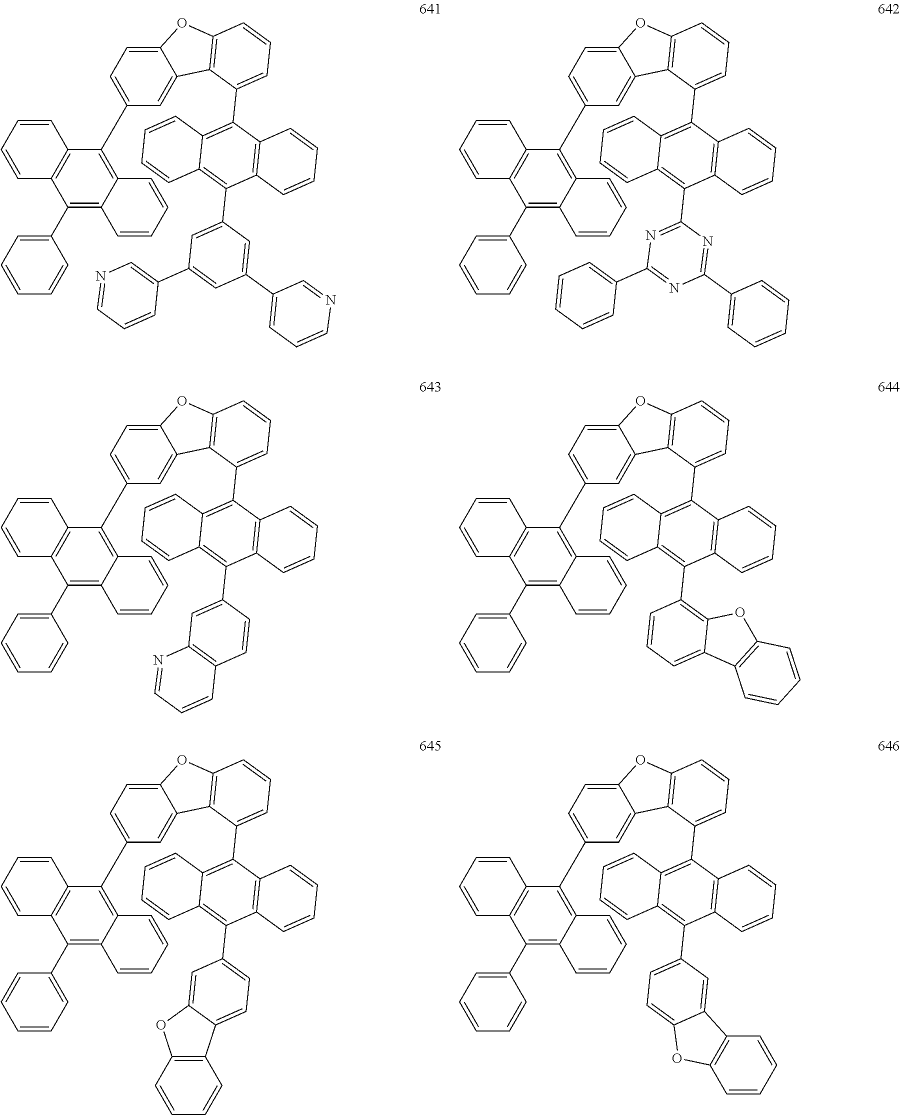

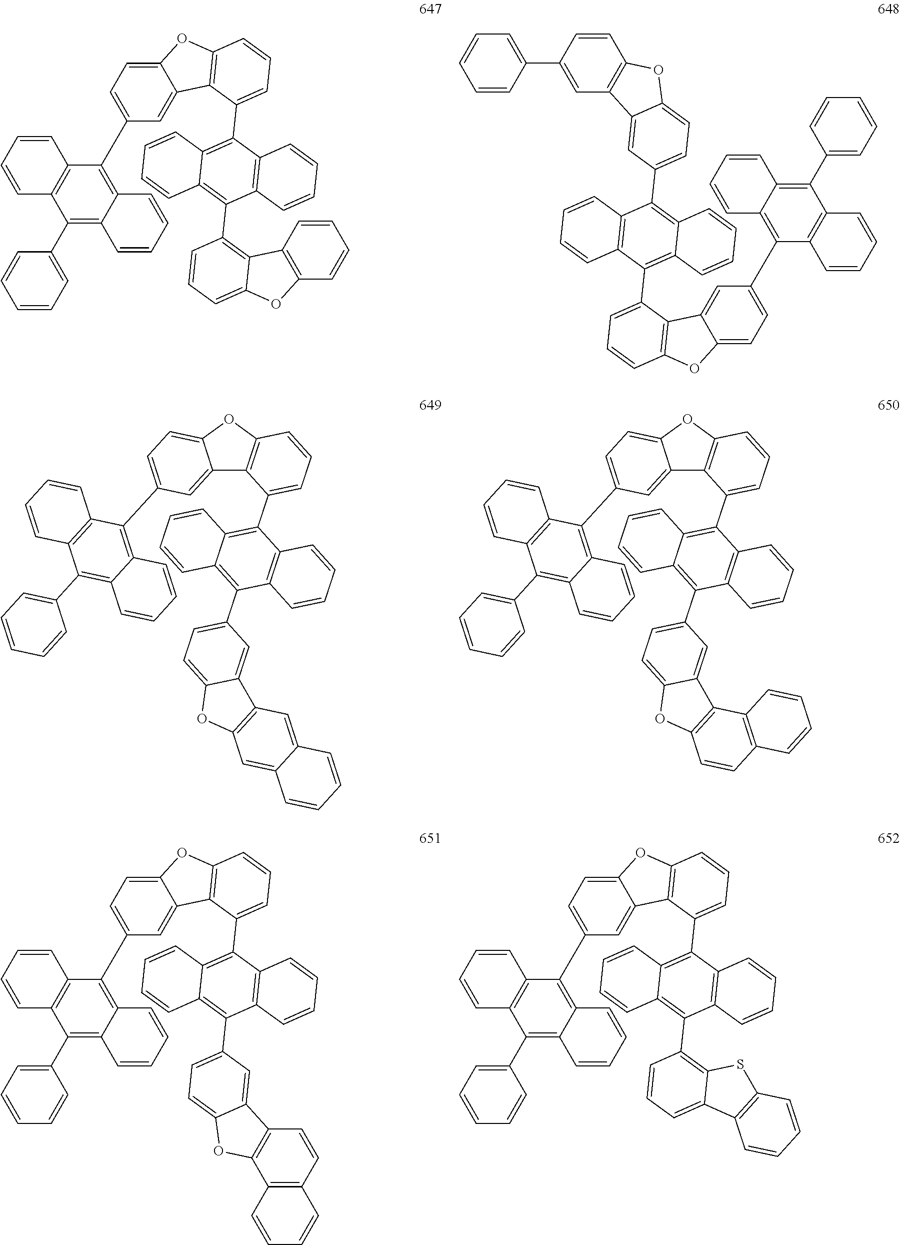

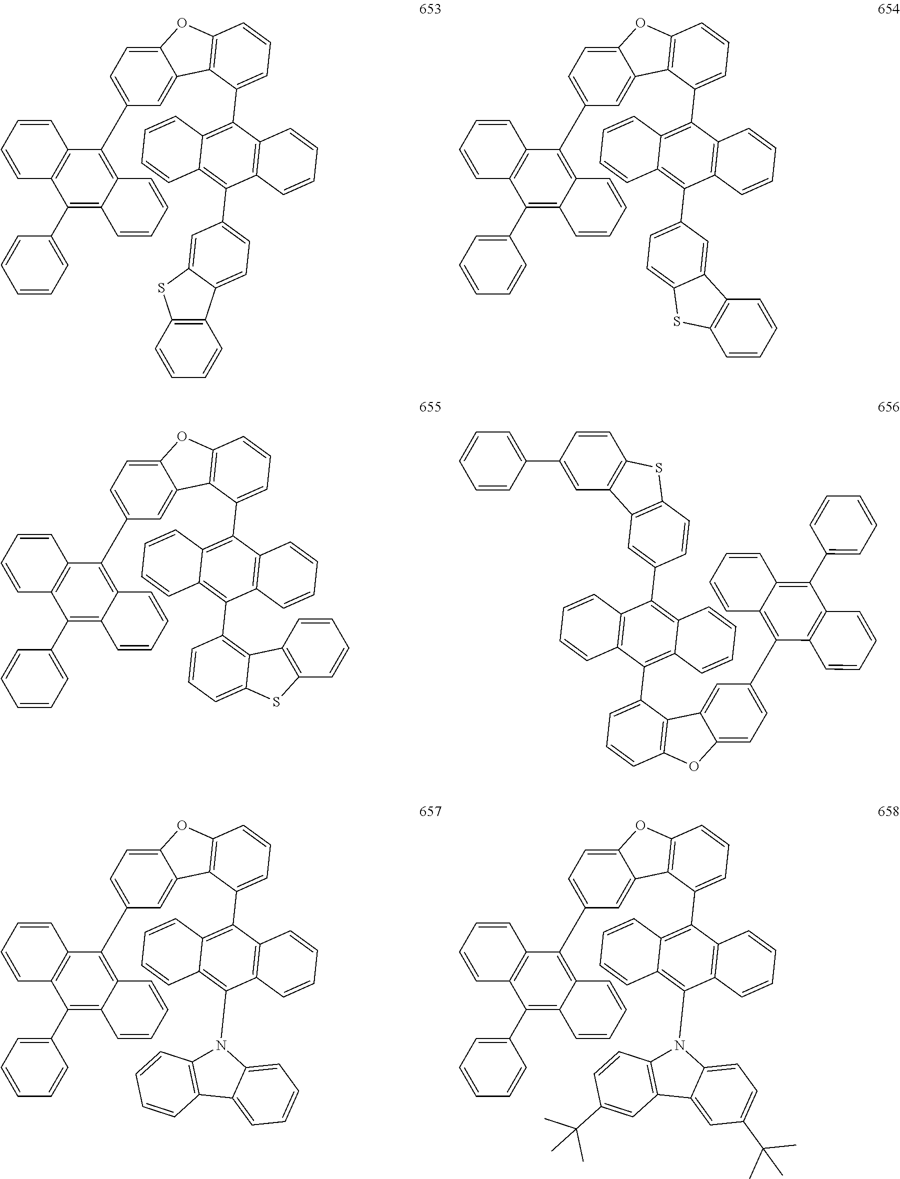

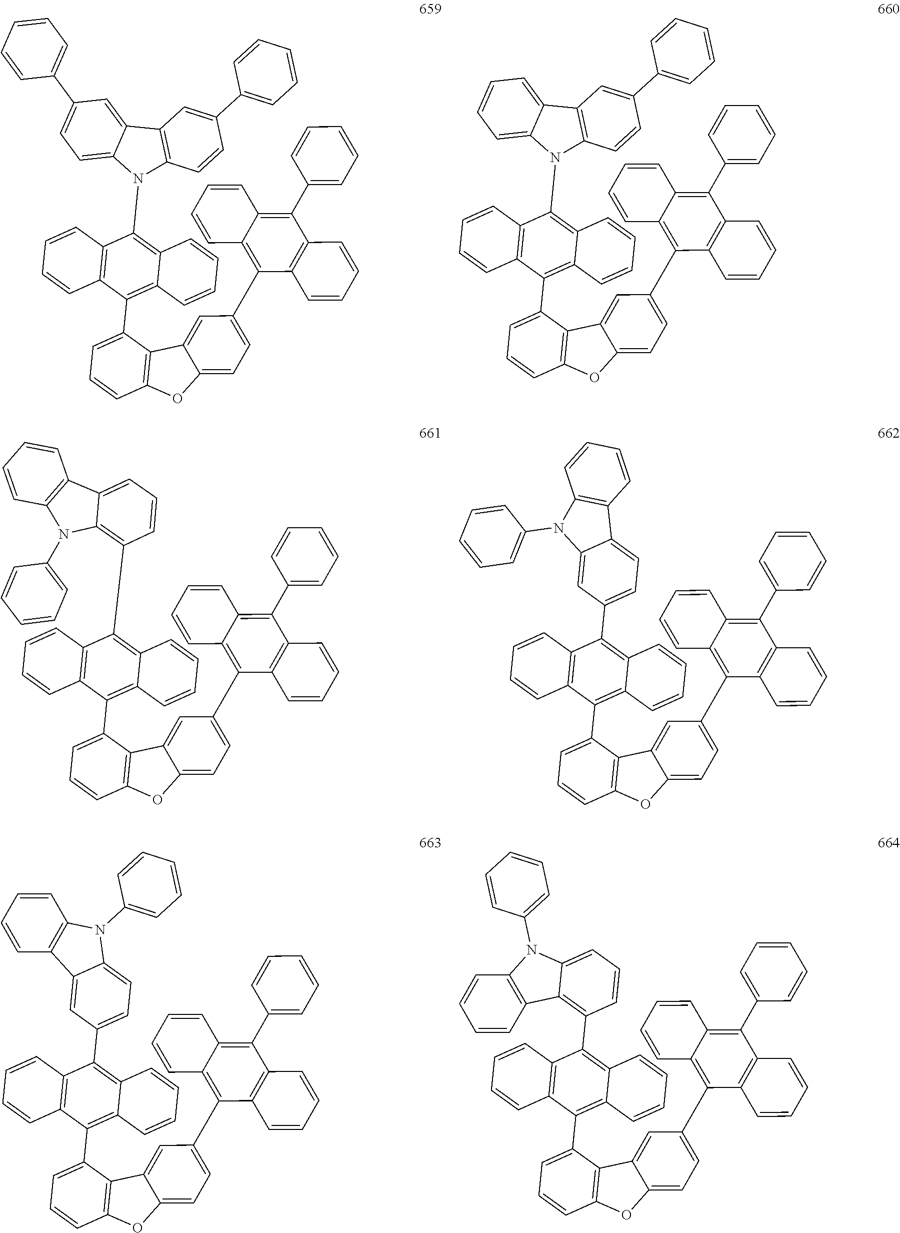

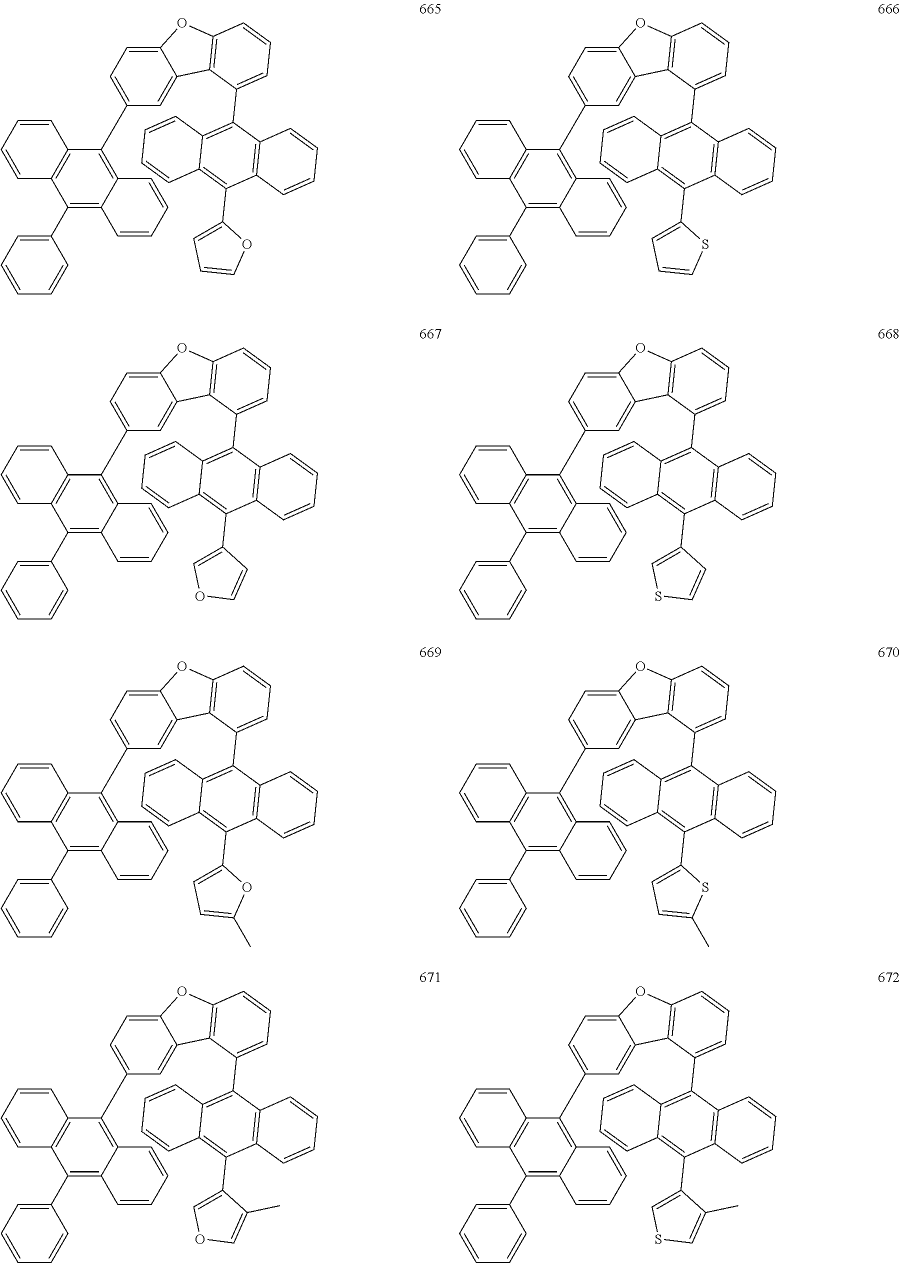

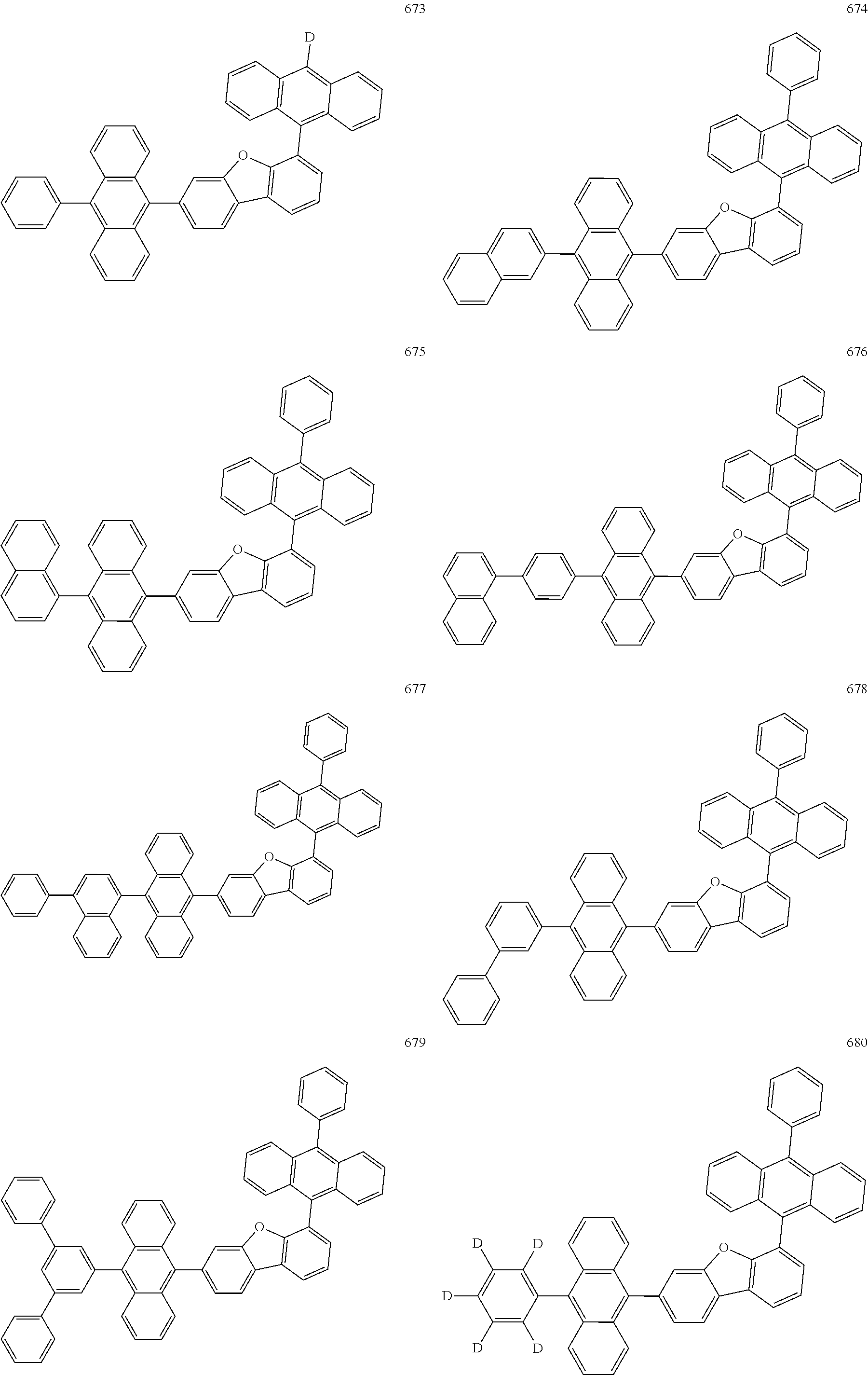

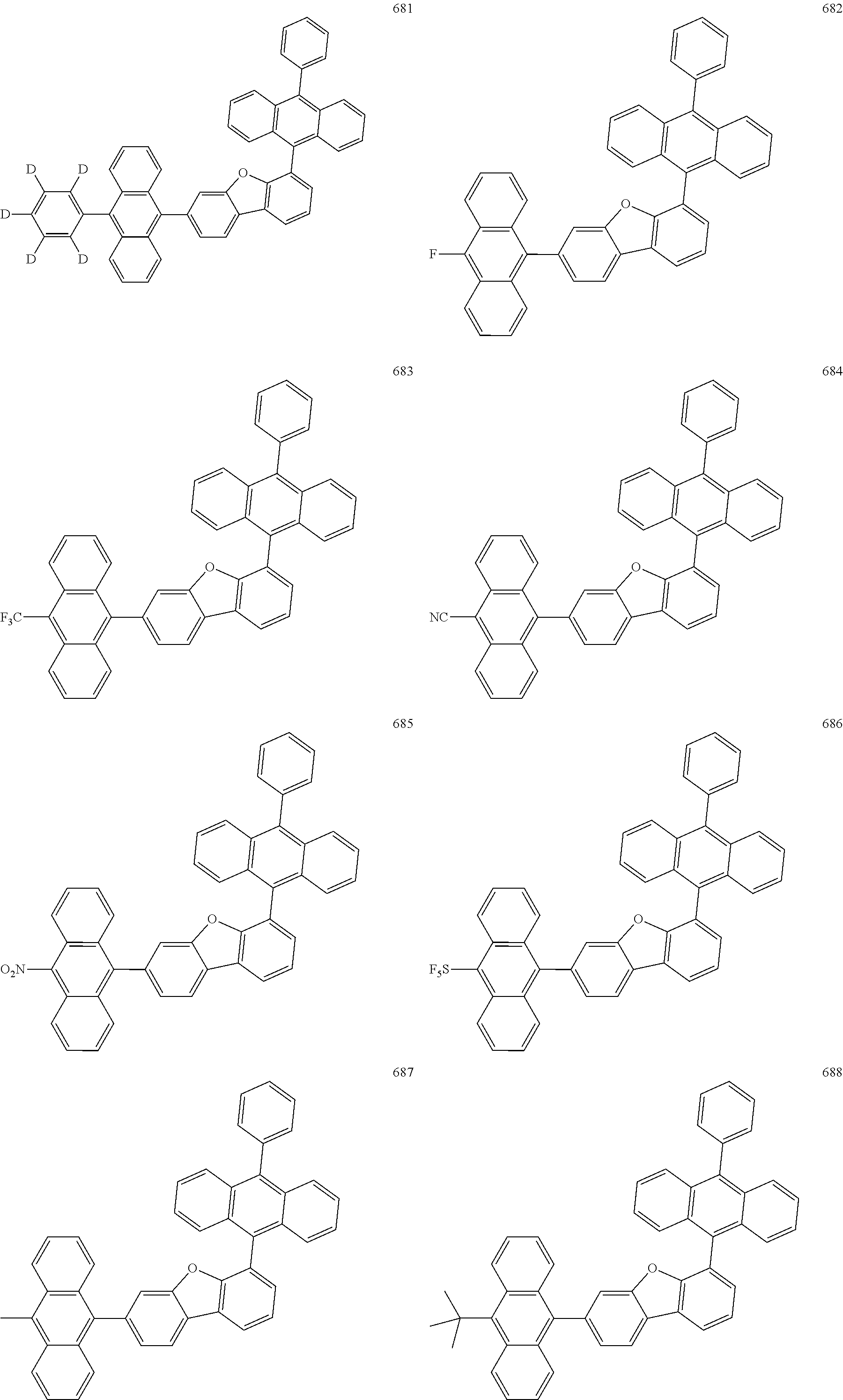

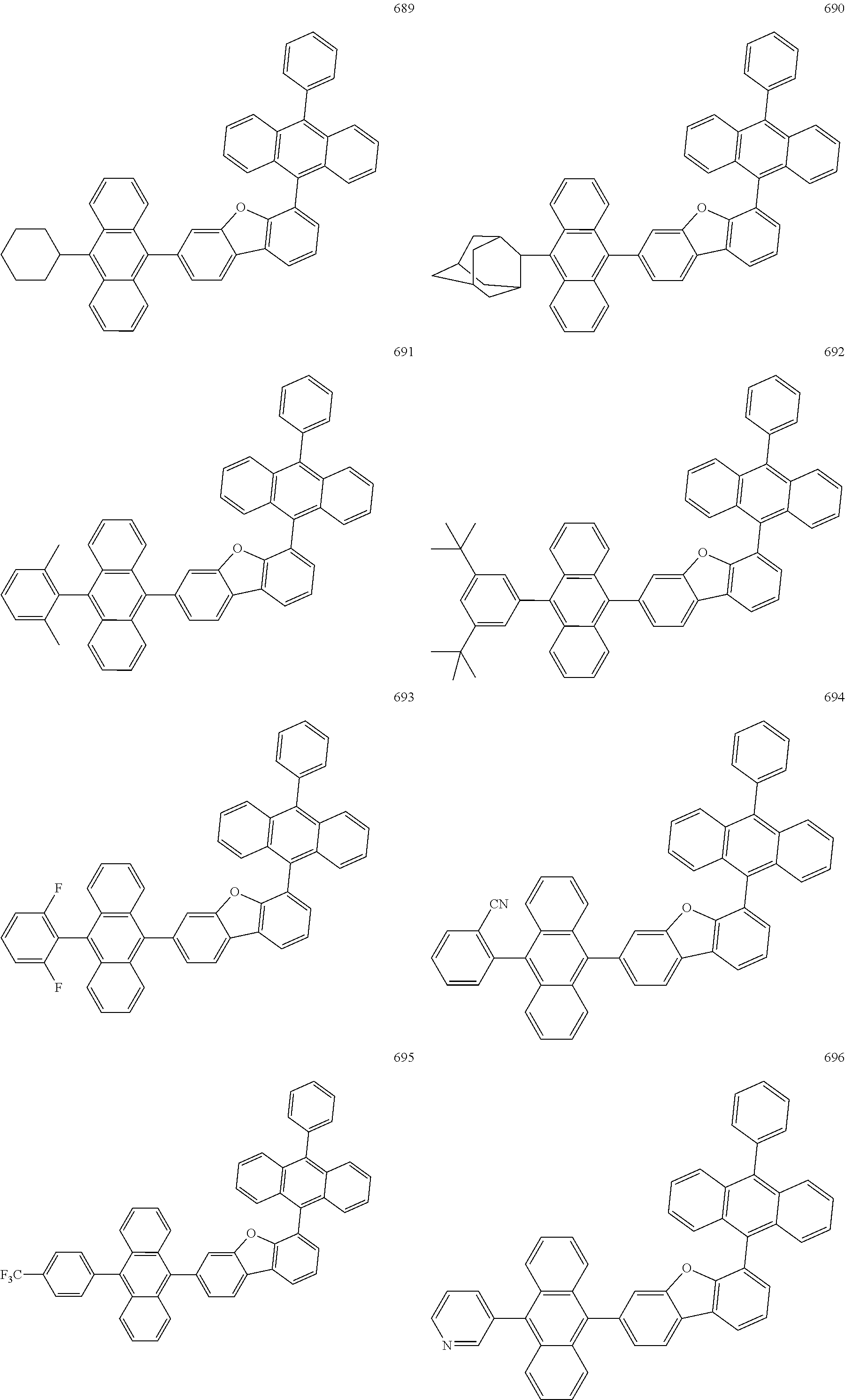

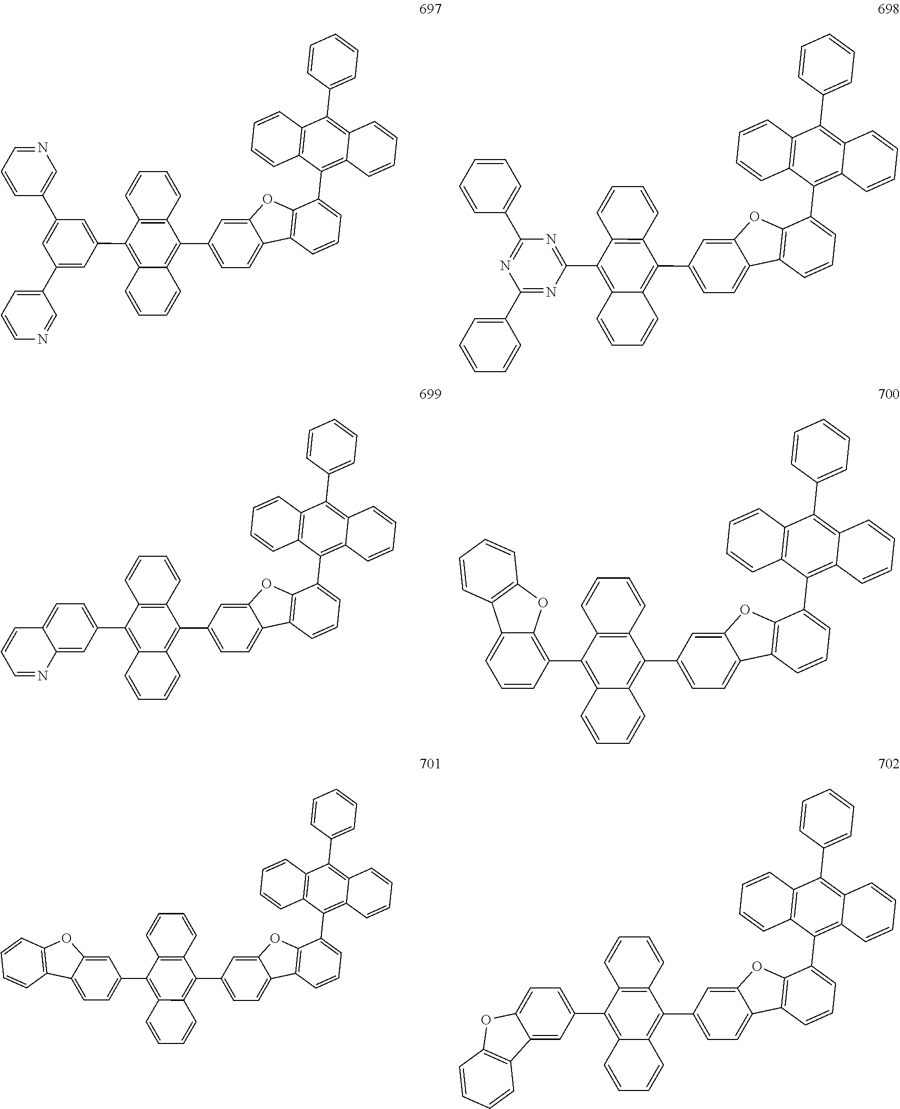

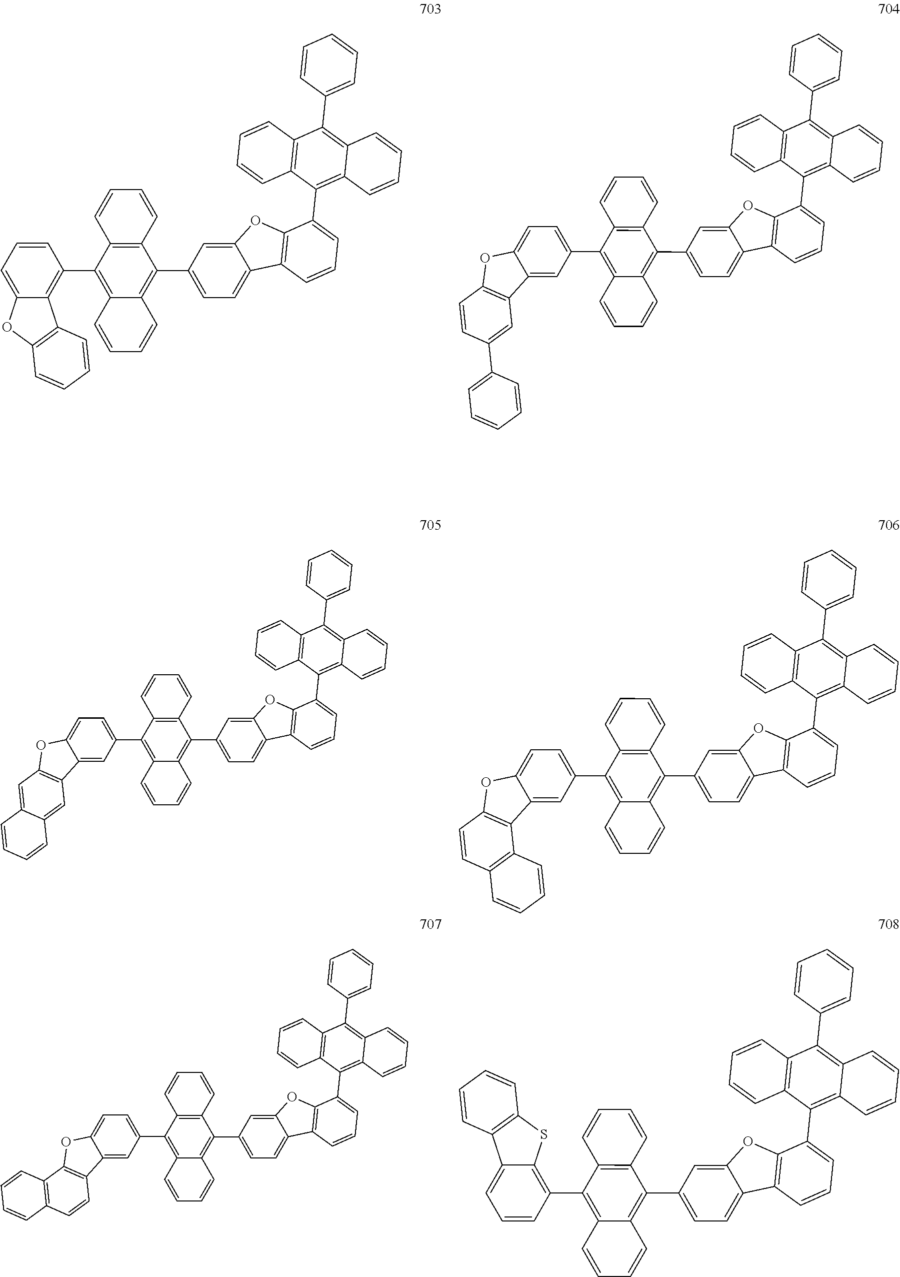

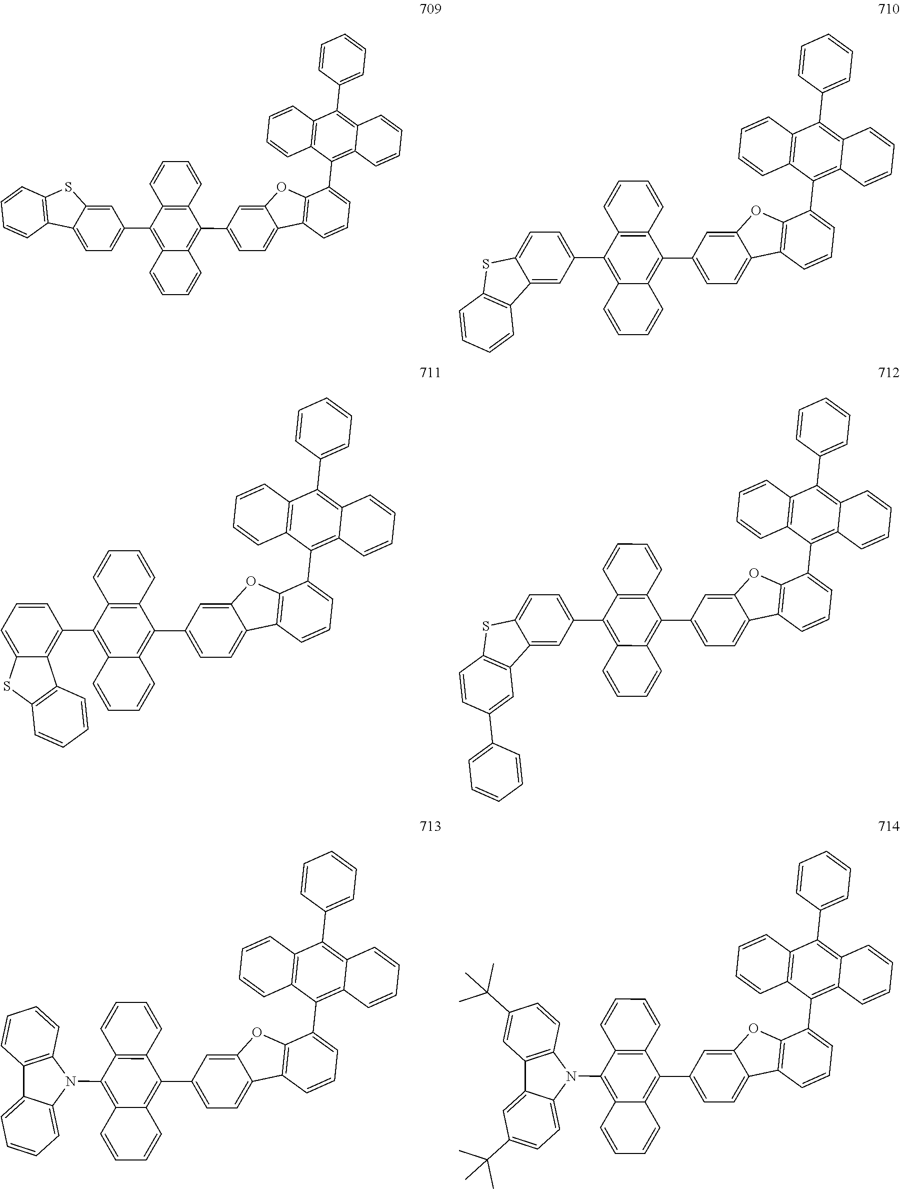

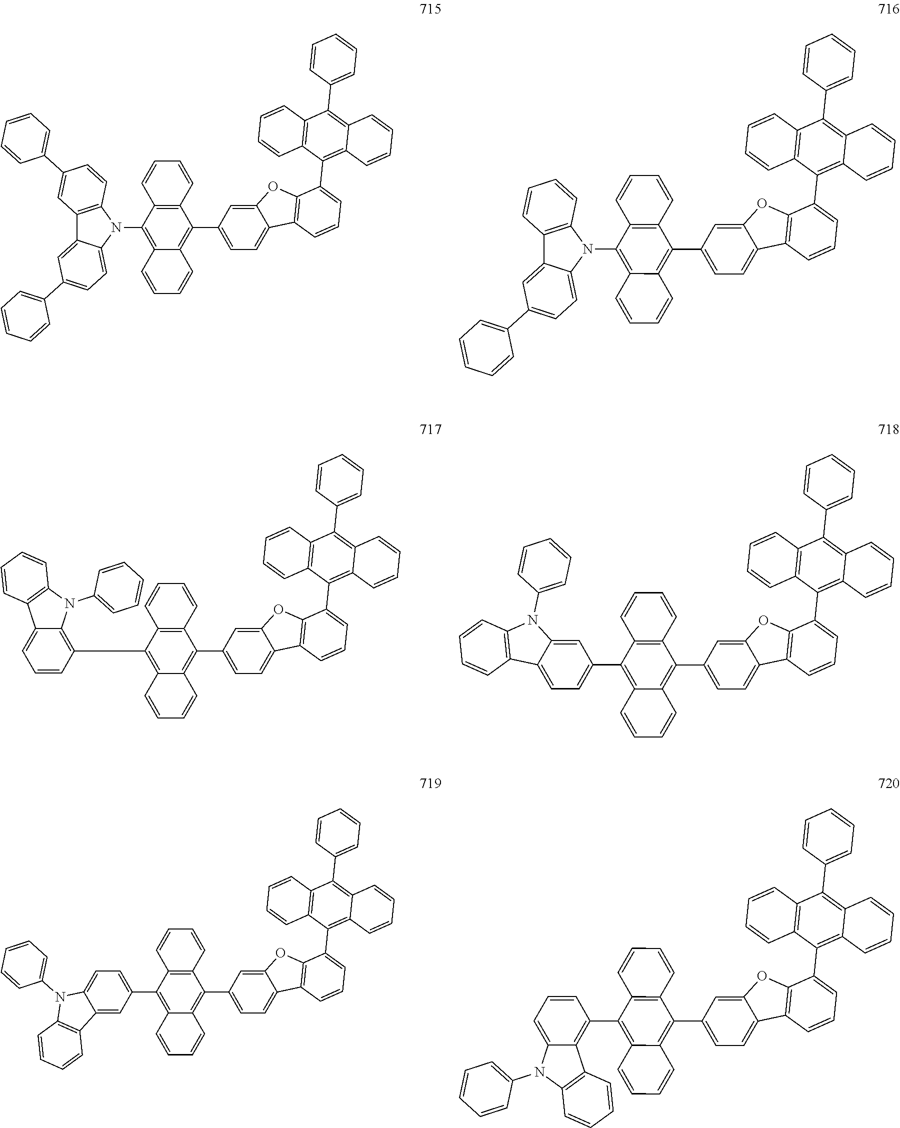

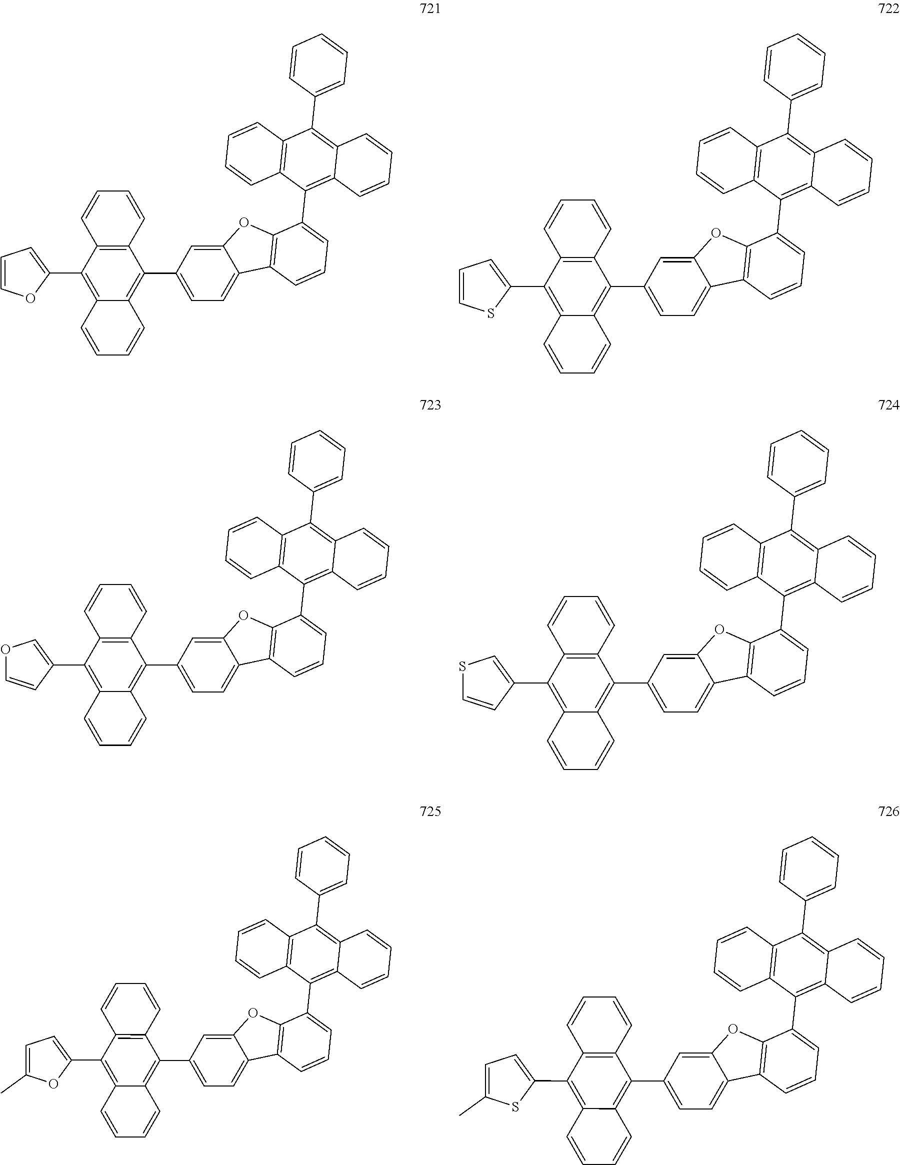

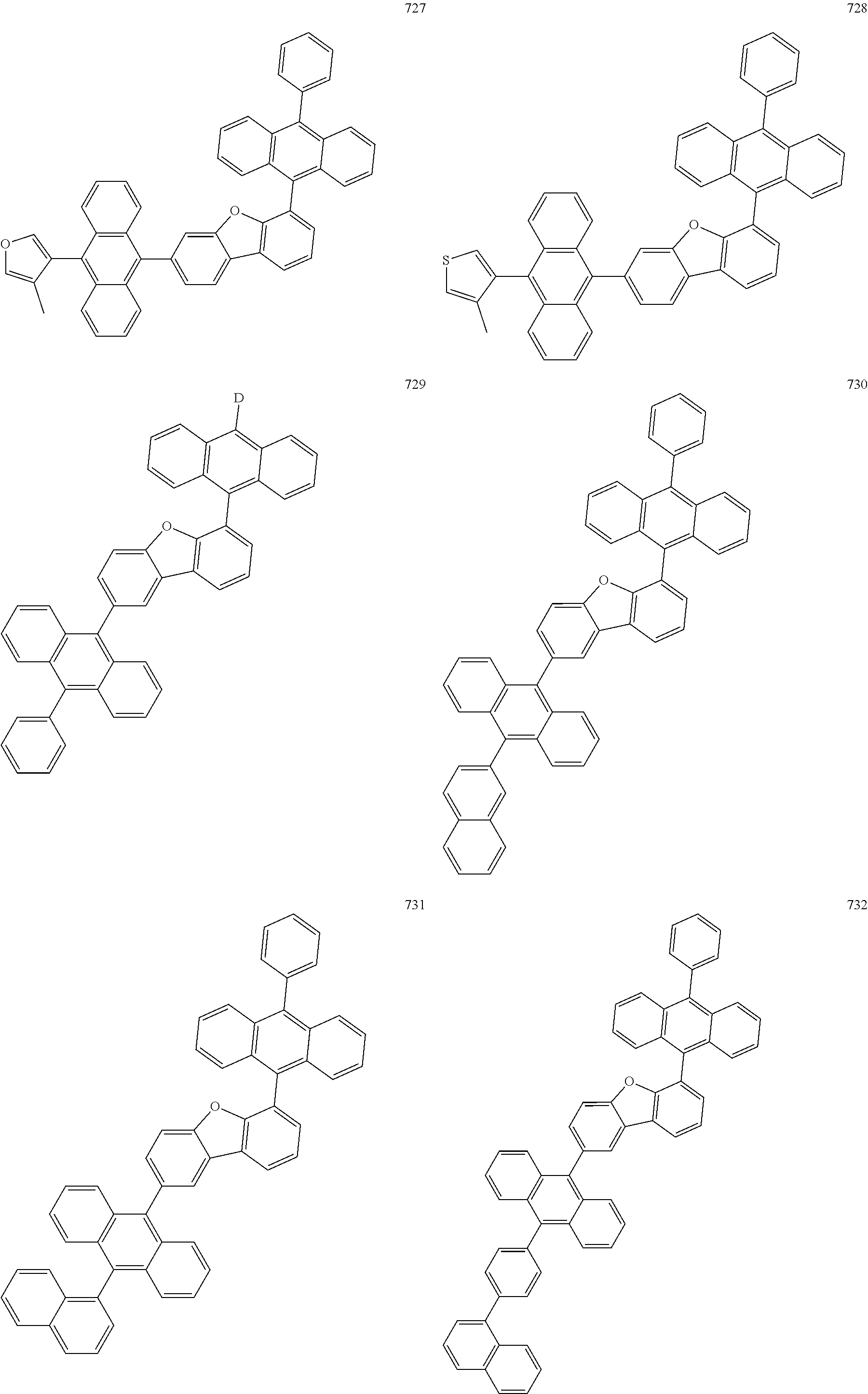

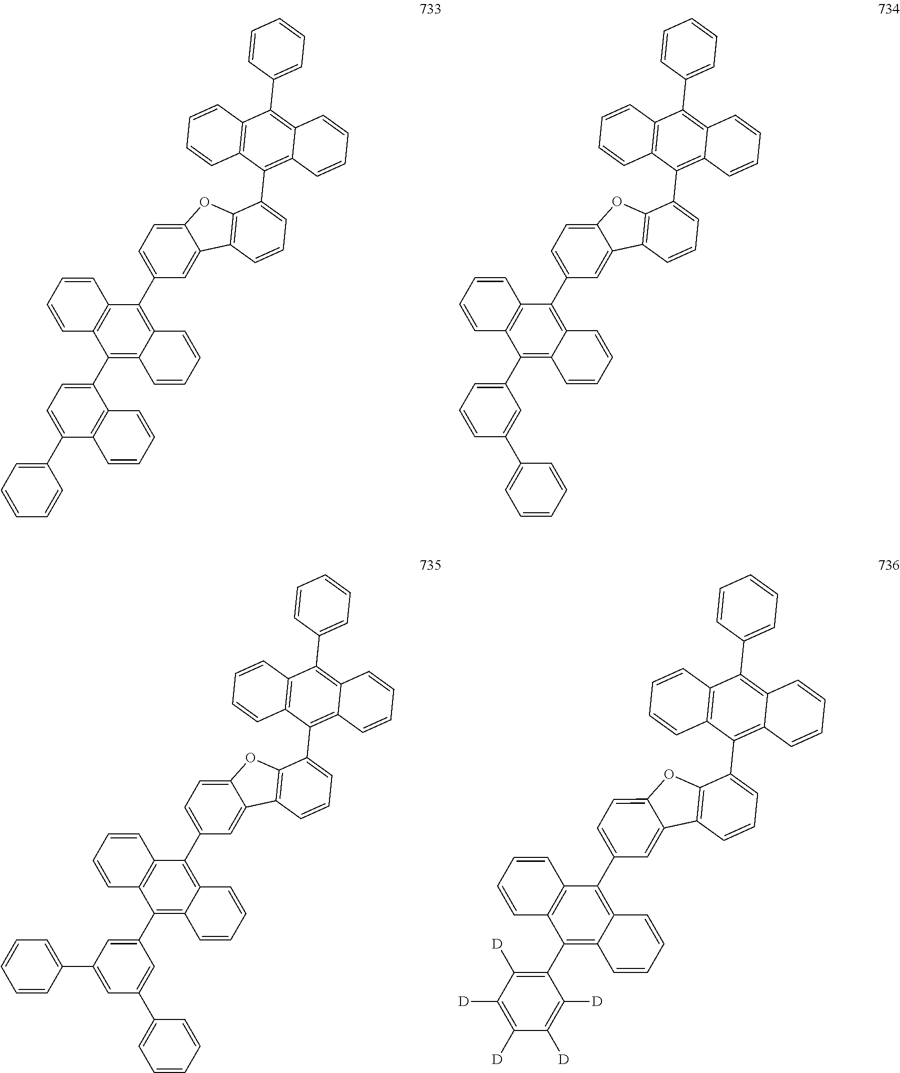 [SMILES: c1ccc(-c2ccc(-c3c4ccccc4c(-c4ccc5oc6c(-c7c8ccccc8c(-c8ccccc8)c8ccccc78)cccc6c5c4)c4ccccc34)c3ccccc23)cc1 ; c1ccc(-c2cccc(-c3c4ccccc4c(-c4ccc5oc6c(-c7c8ccccc8c(-c8ccccc8)c8ccccc78)cccc6c5c4)c4ccccc34)c2)cc1 ; c1ccc(-c2cc(-c3ccccc3)cc(-c3c4ccccc4c(-c4ccc5oc6c(-c7c8ccccc8c(-c8ccccc8)c8ccccc78)cccc6c5c4)c4ccccc34)c2)cc1 ; [2H]c1c([2H])c([2H])c(-c2c3ccccc3c(-c3ccc4oc5c(-c6c7ccccc7c(-c7ccccc7)c7ccccc67)cccc5c4c3)c3ccccc23)c([2H])c1[2H]]

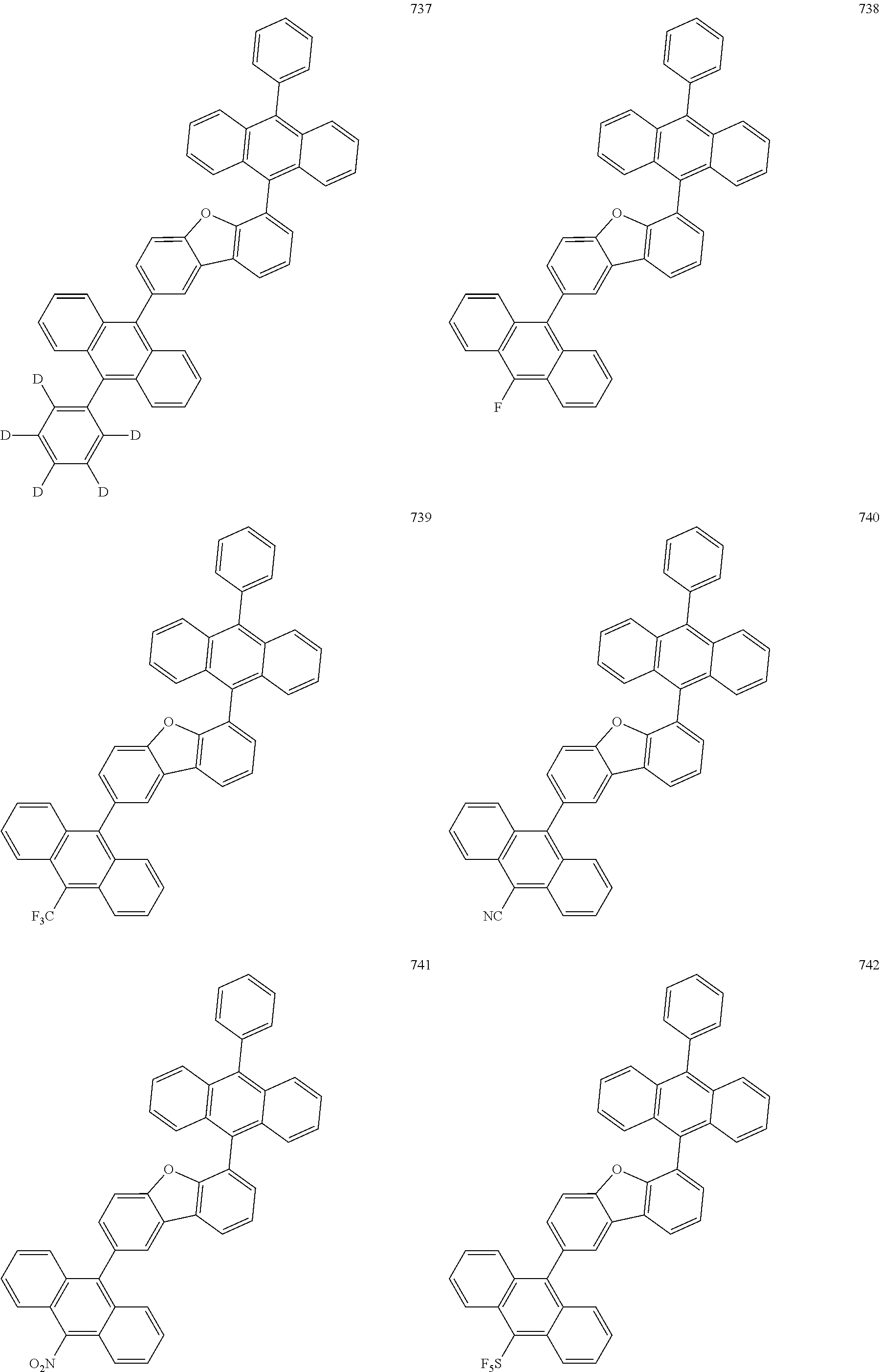

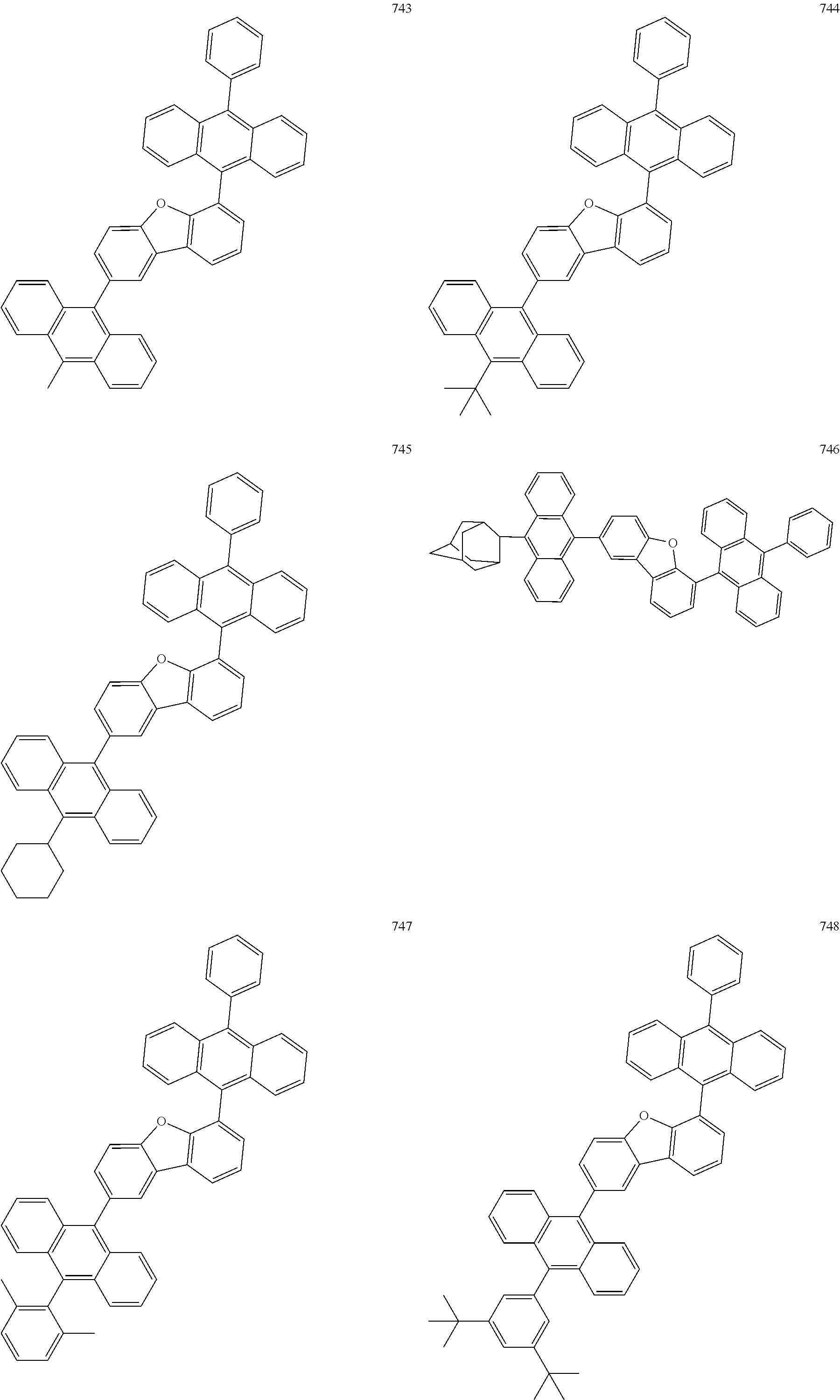

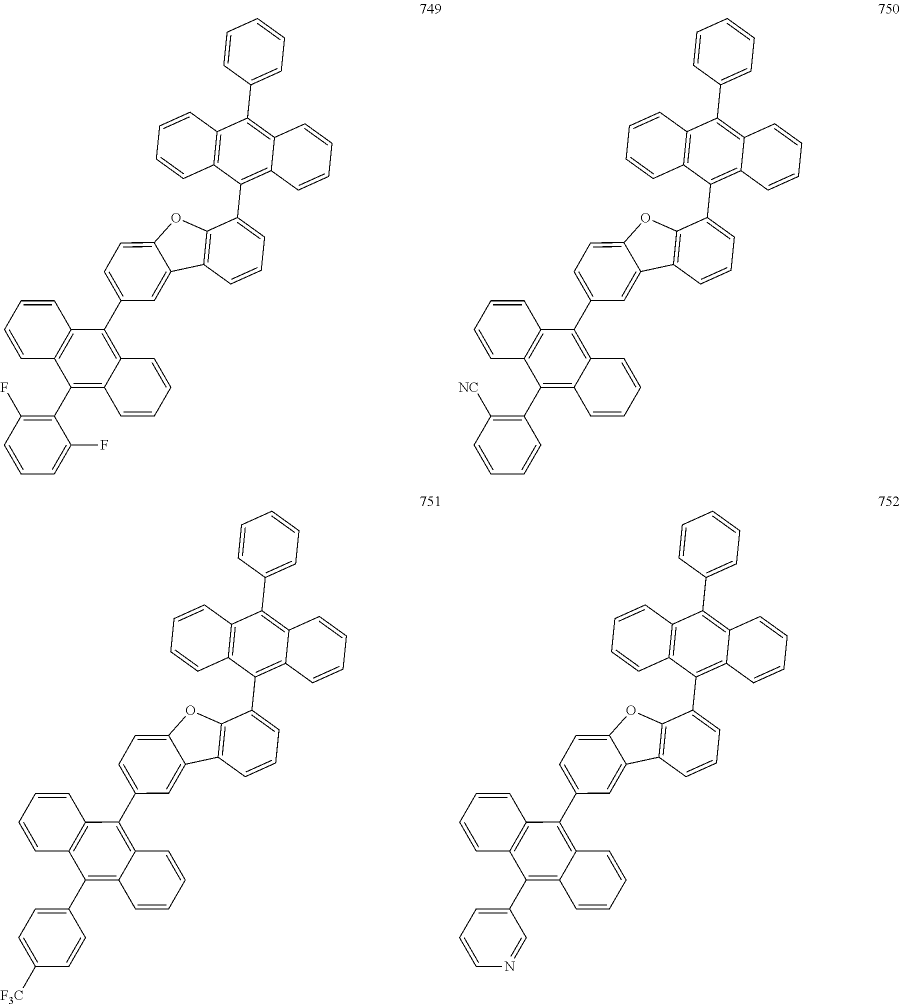 [SMILES: Fc1cccc(F)c1-c1c2ccccc2c(-c2ccc3oc4c(-c5c6ccccc6c(-c6ccccc6)c6ccccc56)cccc4c3c2)c2ccccc12 ; N#Cc1ccccc1-c1c2ccccc2c(-c2ccc3oc4c(-c5c6ccccc6c(-c6ccccc6)c6ccccc56)cccc4c3c2)c2ccccc12 ; FC(F)(F)c1ccc(-c2c3ccccc3c(-c3ccc4oc5c(-c6c7ccccc7c(-c7ccccc7)c7ccccc67)cccc5c4c3)c3ccccc23)cc1 ; c1ccc(-c2c3ccccc3c(-c3cccc4c3oc3ccc(-c5c6ccccc6c(-c6cccnc6)c6ccccc56)cc34)c3ccccc23)cc1]

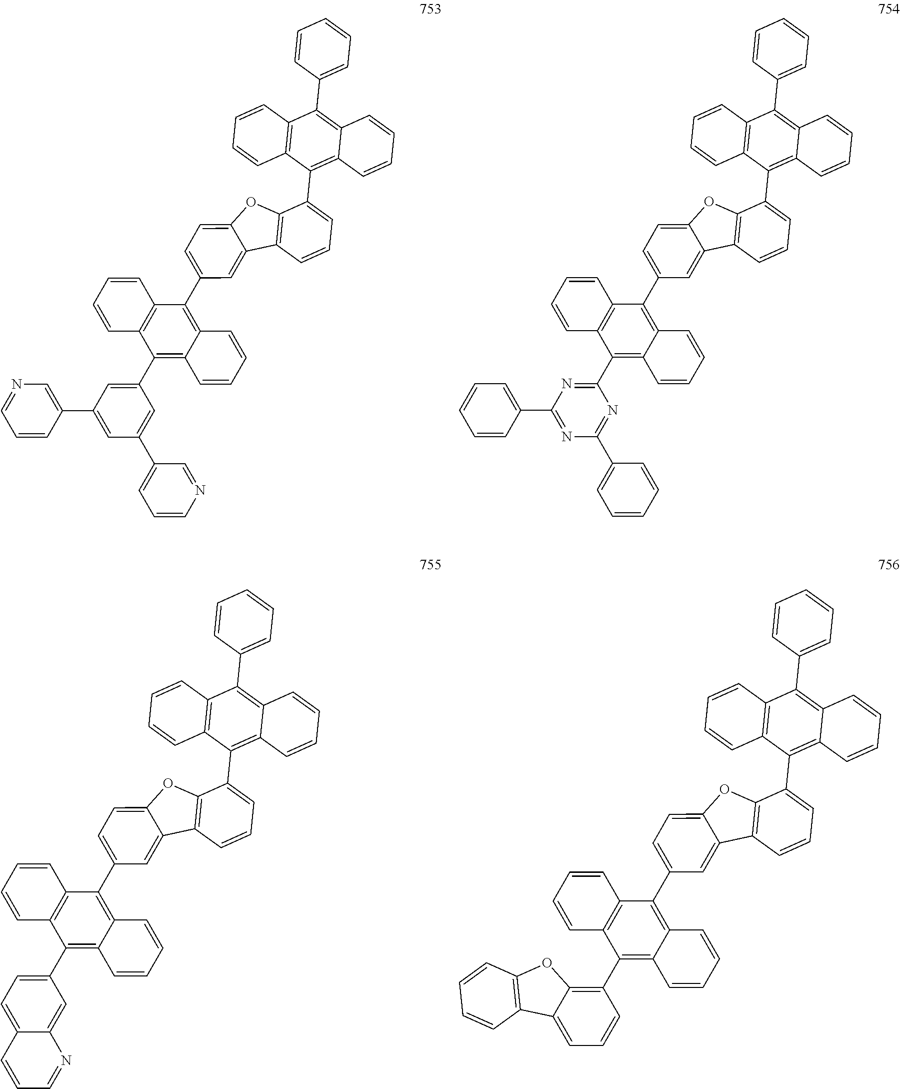 [SMILES: c1ccc(-c2c3ccccc3c(-c3cccc4c3oc3ccc(-c5c6ccccc6c(-c6cc(-c7cccnc7)cc(-c7cccnc7)c6)c6ccccc56)cc34)c3ccccc23)cc1 ; c1ccc(-c2nc(-c3ccccc3)nc(-c3c4ccccc4c(-c4ccc5oc6c(-c7c8ccccc8c(-c8ccccc8)c8ccccc78)cccc6c5c4)c4ccccc34)n2)cc1 ; c1ccc(-c2c3ccccc3c(-c3cccc4c3oc3ccc(-c5c6ccccc6c(-c6ccc7cccnc7c6)c6ccccc56)cc34)c3ccccc23)cc1 ; c1ccc(-c2c3ccccc3c(-c3cccc4c3oc3ccc(-c5c6ccccc6c(-c6cccc7c6oc6ccccc67)c6ccccc56)cc34)c3ccccc23)cc1]

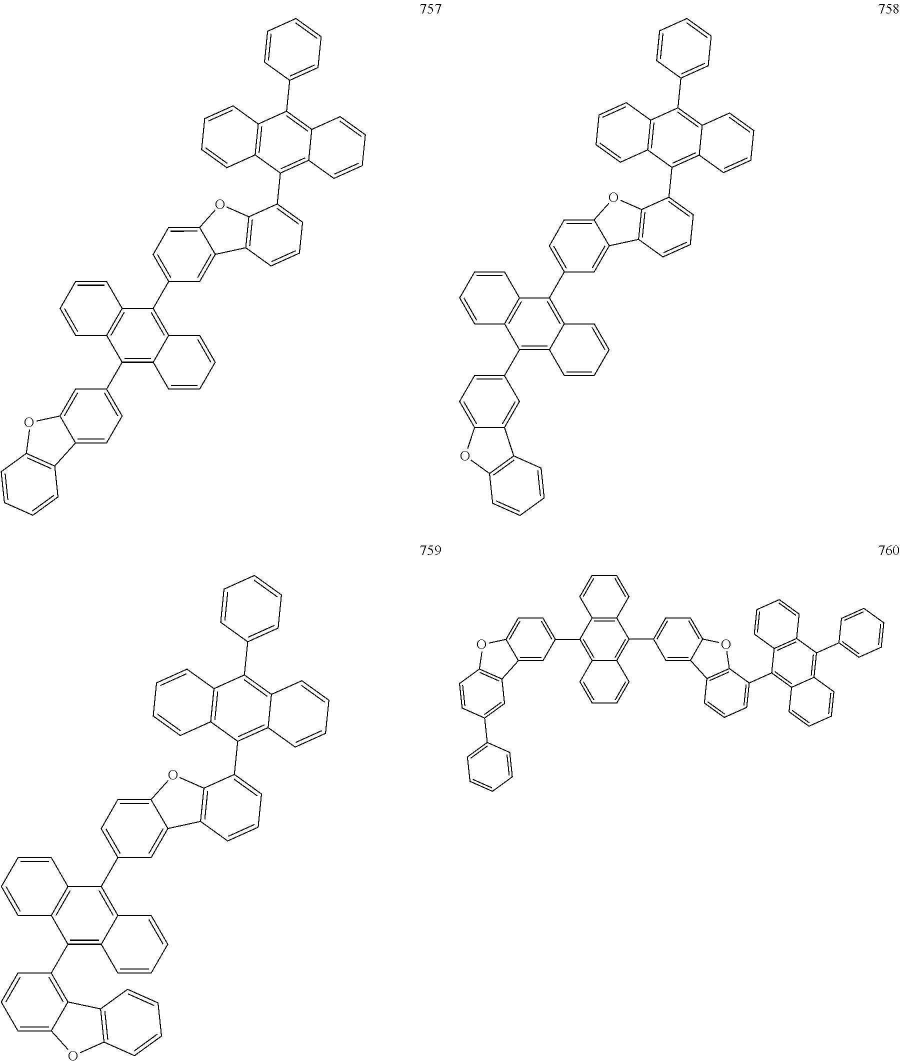 [SMILES: c1ccc(-c2c3ccccc3c(-c3cccc4c3oc3ccc(-c5c6ccccc6c(-c6ccc7c(c6)oc6ccccc67)c6ccccc56)cc34)c3ccccc23)cc1 ; c1ccc(-c2c3ccccc3c(-c3cccc4c3oc3ccc(-c5c6ccccc6c(-c6ccc7oc8ccccc8c7c6)c6ccccc56)cc34)c3ccccc23)cc1 ; c1ccc(-c2c3ccccc3c(-c3cccc4c3oc3ccc(-c5c6ccccc6c(-c6cccc7oc8ccccc8c67)c6ccccc56)cc34)c3ccccc23)cc1 ; c1ccc(-c2ccc3oc4ccc(-c5c6ccccc6c(-c6ccc7oc8c(-c9c%10ccccc%10c(-c%10ccccc%10)c%10ccccc9%10)cccc8c7c6)c6ccccc56)cc4c3c2)cc1]

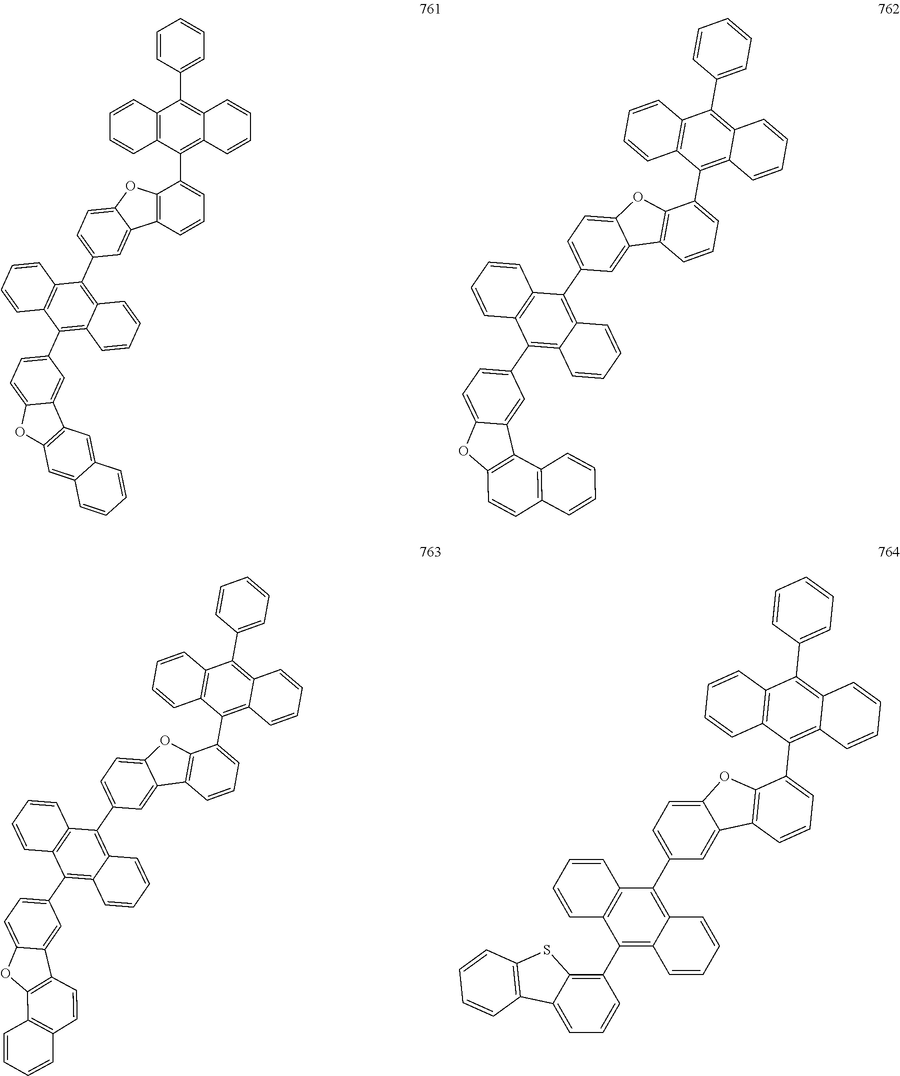 [SMILES: c1ccc(-c2c3ccccc3c(-c3cccc4c3oc3ccc(-c5c6ccccc6c(-c6ccc7oc8cc9ccccc9cc8c7c6)c6ccccc56)cc34)c3ccccc23)cc1 ; c1ccc(-c2c3ccccc3c(-c3cccc4c3oc3ccc(-c5c6ccccc6c(-c6ccc7oc8ccc9ccccc9c8c7c6)c6ccccc56)cc34)c3ccccc23)cc1 ; c1ccc(-c2c3ccccc3c(-c3cccc4c3oc3ccc(-c5c6ccccc6c(-c6ccc7oc8c9ccccc9ccc8c7c6)c6ccccc56)cc34)c3ccccc23)cc1 ; c1ccc(-c2c3ccccc3c(-c3cccc4c3oc3ccc(-c5c6ccccc6c(-c6cccc7c6sc6ccccc67)c6ccccc56)cc34)c3ccccc23)cc1]

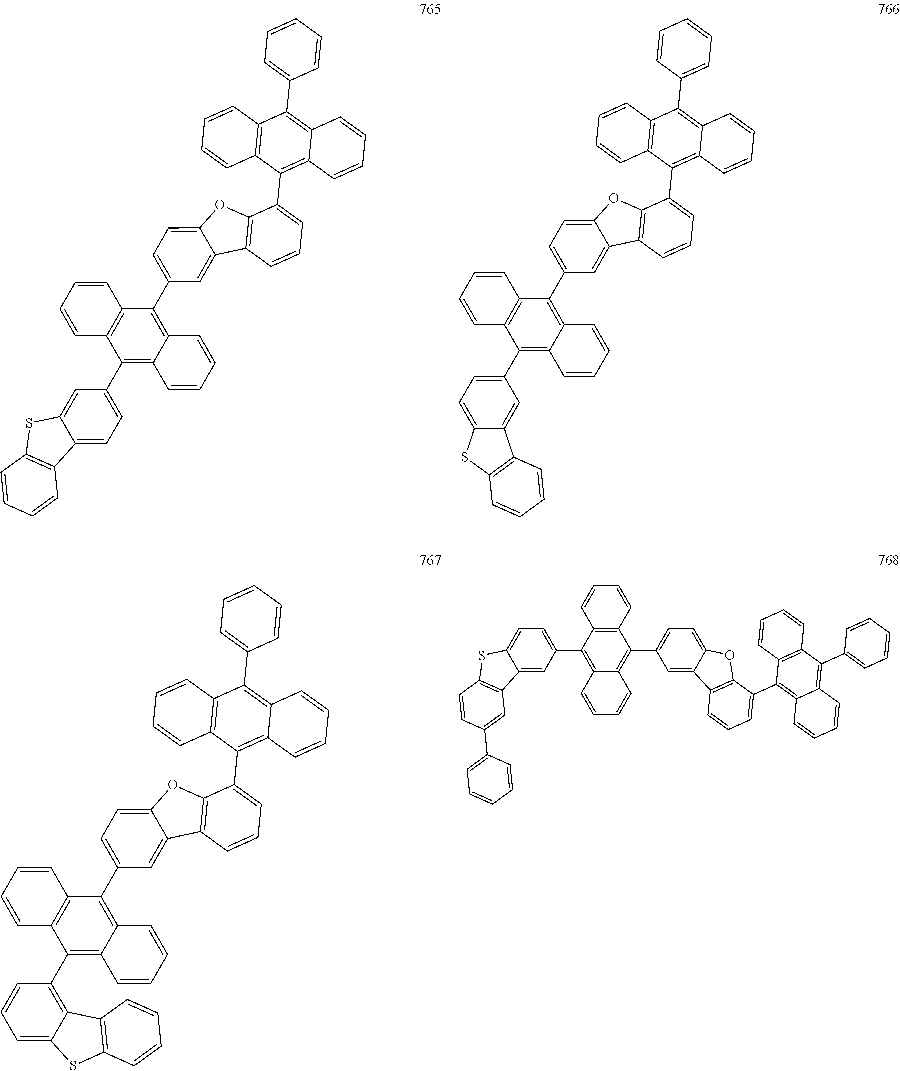 [SMILES: c1ccc(-c2c3ccccc3c(-c3cccc4c3oc3ccc(-c5c6ccccc6c(-c6ccc7c(c6)sc6ccccc67)c6ccccc56)cc34)c3ccccc23)cc1 ; c1ccc(-c2c3ccccc3c(-c3cccc4c3oc3ccc(-c5c6ccccc6c(-c6ccc7sc8ccccc8c7c6)c6ccccc56)cc34)c3ccccc23)cc1 ; c1ccc(-c2c3ccccc3c(-c3cccc4c3oc3ccc(-c5c6ccccc6c(-c6cccc7sc8ccccc8c67)c6ccccc56)cc34)c3ccccc23)cc1 ; c1ccc(-c2ccc3sc4ccc(-c5c6ccccc6c(-c6ccc7oc8c(-c9c%10ccccc%10c(-c%10ccccc%10)c%10ccccc9%10)cccc8c7c6)c6ccccc56)cc4c3c2)cc1]

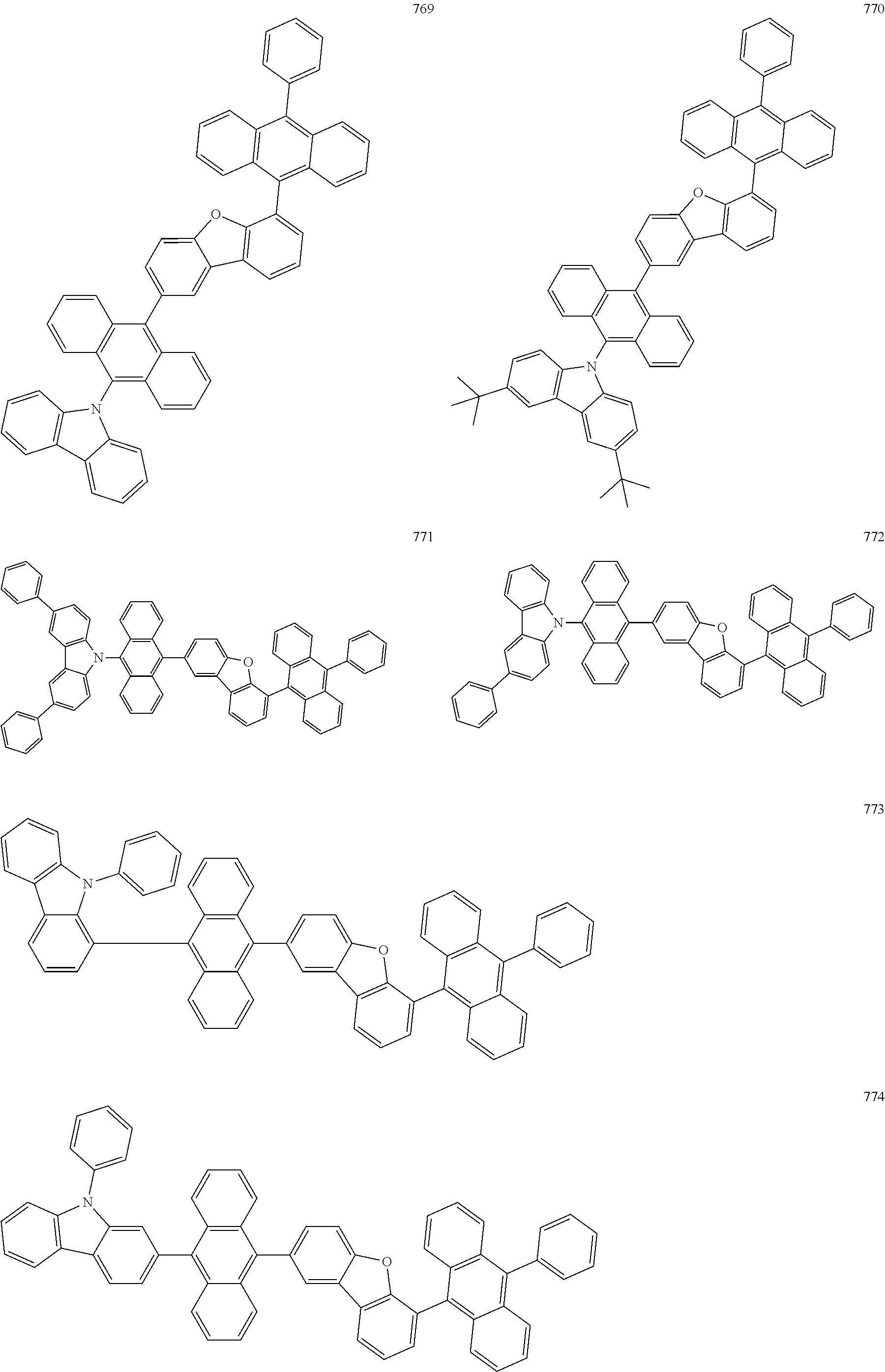

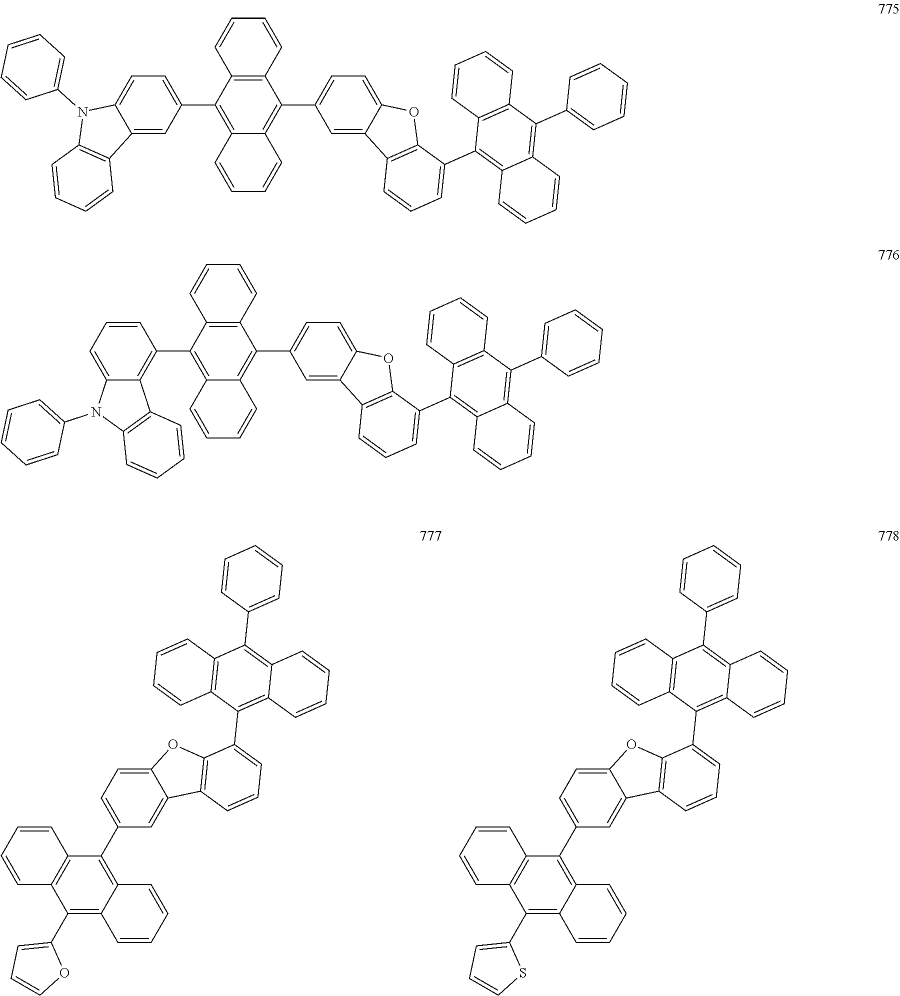 [SMILES: c1ccc(-c2c3ccccc3c(-c3cccc4c3oc3ccc(-c5c6ccccc6c(-c6ccc7c(c6)c6ccccc6n7-c6ccccc6)c6ccccc56)cc34)c3ccccc23)cc1 ; c1ccc(-c2c3ccccc3c(-c3cccc4c3oc3ccc(-c5c6ccccc6c(-c6cccc7c6c6ccccc6n7-c6ccccc6)c6ccccc56)cc34)c3ccccc23)cc1 ; c1ccc(-c2c3ccccc3c(-c3cccc4c3oc3ccc(-c5c6ccccc6c(-c6ccco6)c6ccccc56)cc34)c3ccccc23)cc1 ; c1ccc(-c2c3ccccc3c(-c3cccc4c3oc3ccc(-c5c6ccccc6c(-c6cccs6)c6ccccc56)cc34)c3ccccc23)cc1]

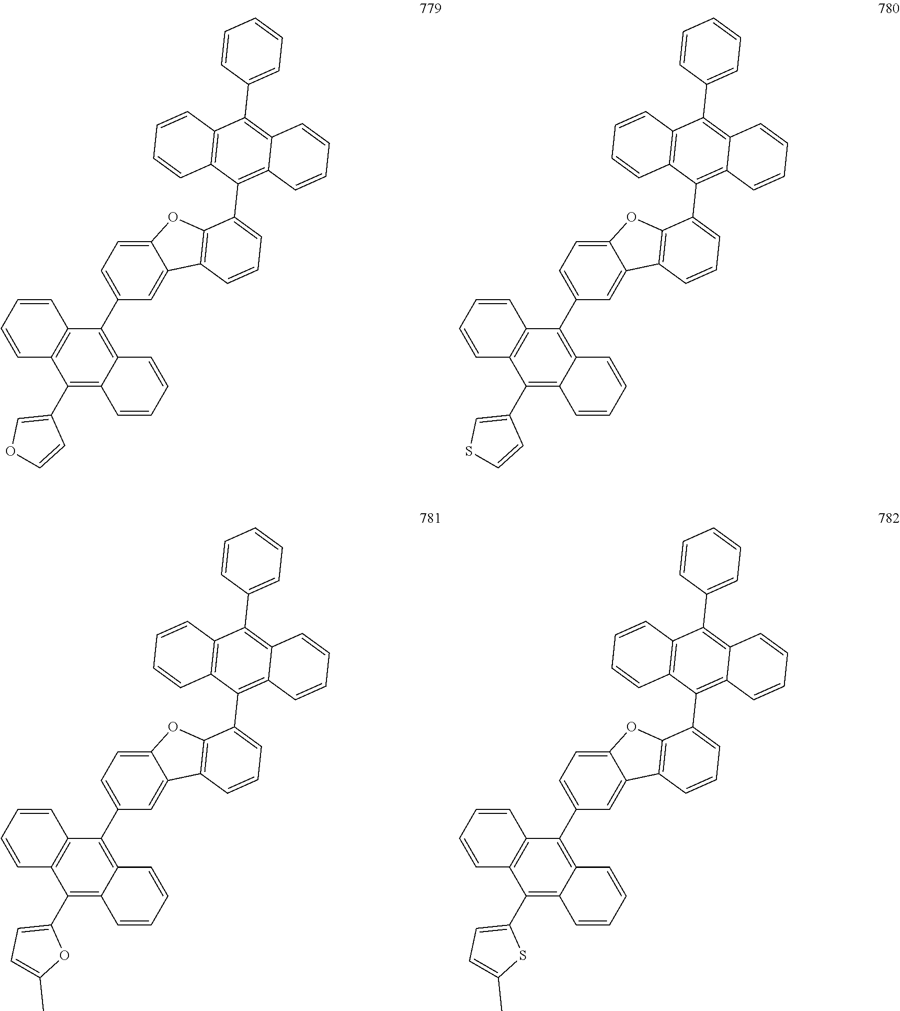 [SMILES: c1ccc(-c2c3ccccc3c(-c3cccc4c3oc3ccc(-c5c6ccccc6c(-c6ccoc6)c6ccccc56)cc34)c3ccccc23)cc1 ; c1ccc(-c2c3ccccc3c(-c3cccc4c3oc3ccc(-c5c6ccccc6c(-c6ccsc6)c6ccccc56)cc34)c3ccccc23)cc1 ; Cc1ccc(-c2c3ccccc3c(-c3ccc4oc5c(-c6c7ccccc7c(-c7ccccc7)c7ccccc67)cccc5c4c3)c3ccccc23)o1 ; Cc1ccc(-c2c3ccccc3c(-c3ccc4oc5c(-c6c7ccccc7c(-c7ccccc7)c7ccccc67)cccc5c4c3)c3ccccc23)s1]

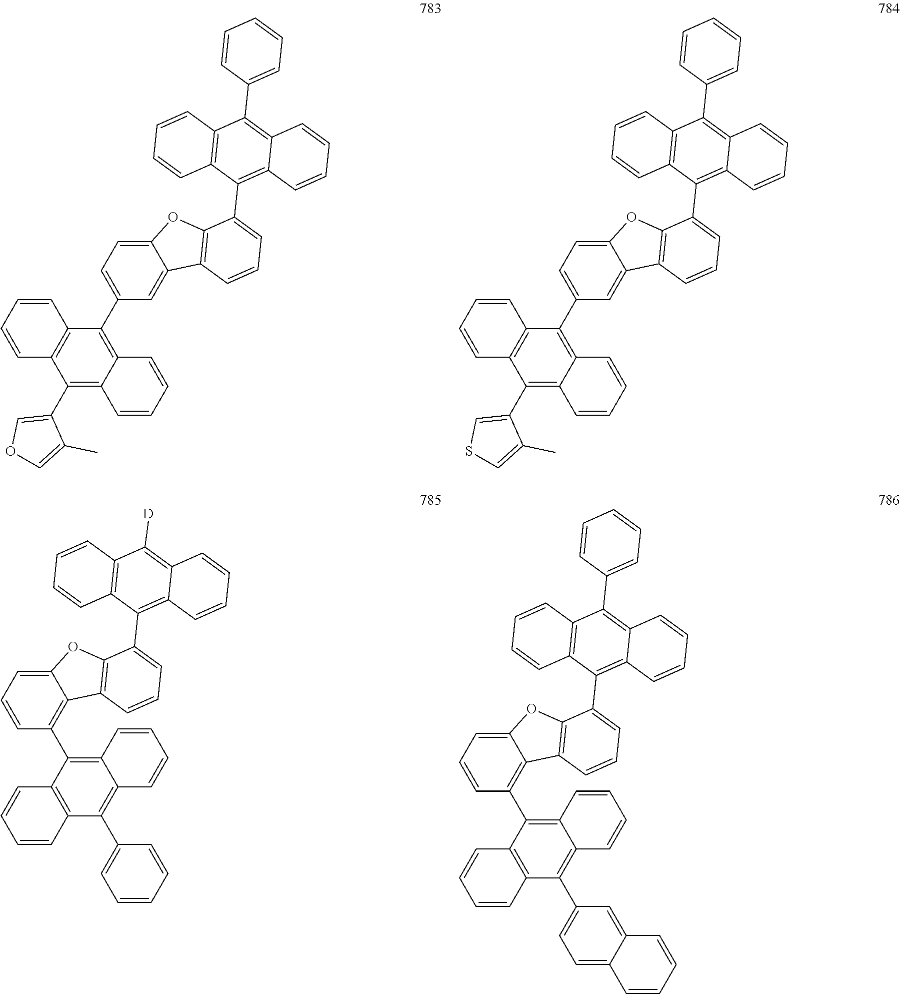 [SMILES: Cc1cocc1-c1c2ccccc2c(-c2ccc3oc4c(-c5c6ccccc6c(-c6ccccc6)c6ccccc56)cccc4c3c2)c2ccccc12 ; Cc1cscc1-c1c2ccccc2c(-c2ccc3oc4c(-c5c6ccccc6c(-c6ccccc6)c6ccccc56)cccc4c3c2)c2ccccc12 ; [2H]c1c2ccccc2c(-c2cccc3c2oc2cccc(-c4c5ccccc5c(-c5ccccc5)c5ccccc45)c23)c2ccccc12 ; c1ccc(-c2c3ccccc3c(-c3cccc4c3oc3cccc(-c5c6ccccc6c(-c6ccc7ccccc7c6)c6ccccc56)c34)c3ccccc23)cc1]

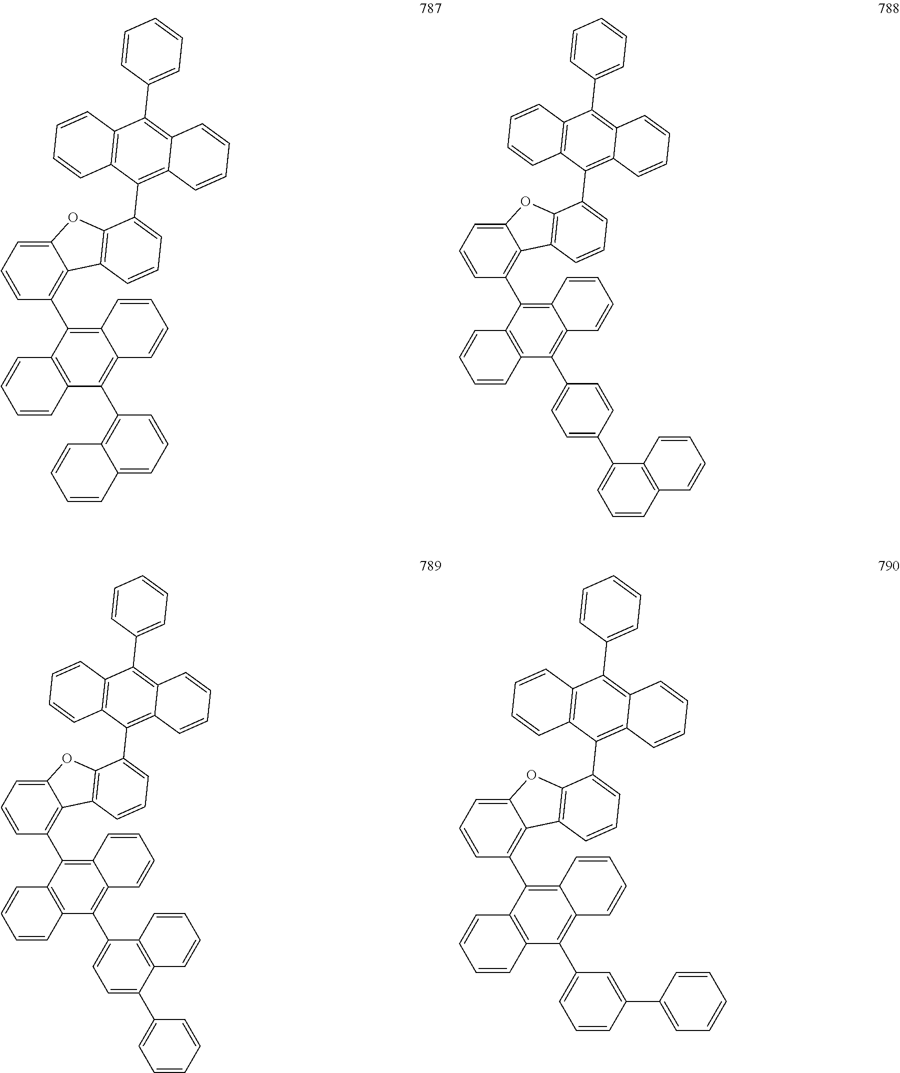 [SMILES: c1ccc(-c2c3ccccc3c(-c3cccc4c3oc3cccc(-c5c6ccccc6c(-c6cccc7ccccc67)c6ccccc56)c34)c3ccccc23)cc1 ; c1ccc(-c2c3ccccc3c(-c3cccc4c3oc3cccc(-c5c6ccccc6c(-c6ccc(-c7cccc8ccccc78)cc6)c6ccccc56)c34)c3ccccc23)cc1 ; c1ccc(-c2ccc(-c3c4ccccc4c(-c4cccc5oc6c(-c7c8ccccc8c(-c8ccccc8)c8ccccc78)cccc6c45)c4ccccc34)c3ccccc23)cc1 ; c1ccc(-c2cccc(-c3c4ccccc4c(-c4cccc5oc6c(-c7c8ccccc8c(-c8ccccc8)c8ccccc78)cccc6c45)c4ccccc34)c2)cc1]

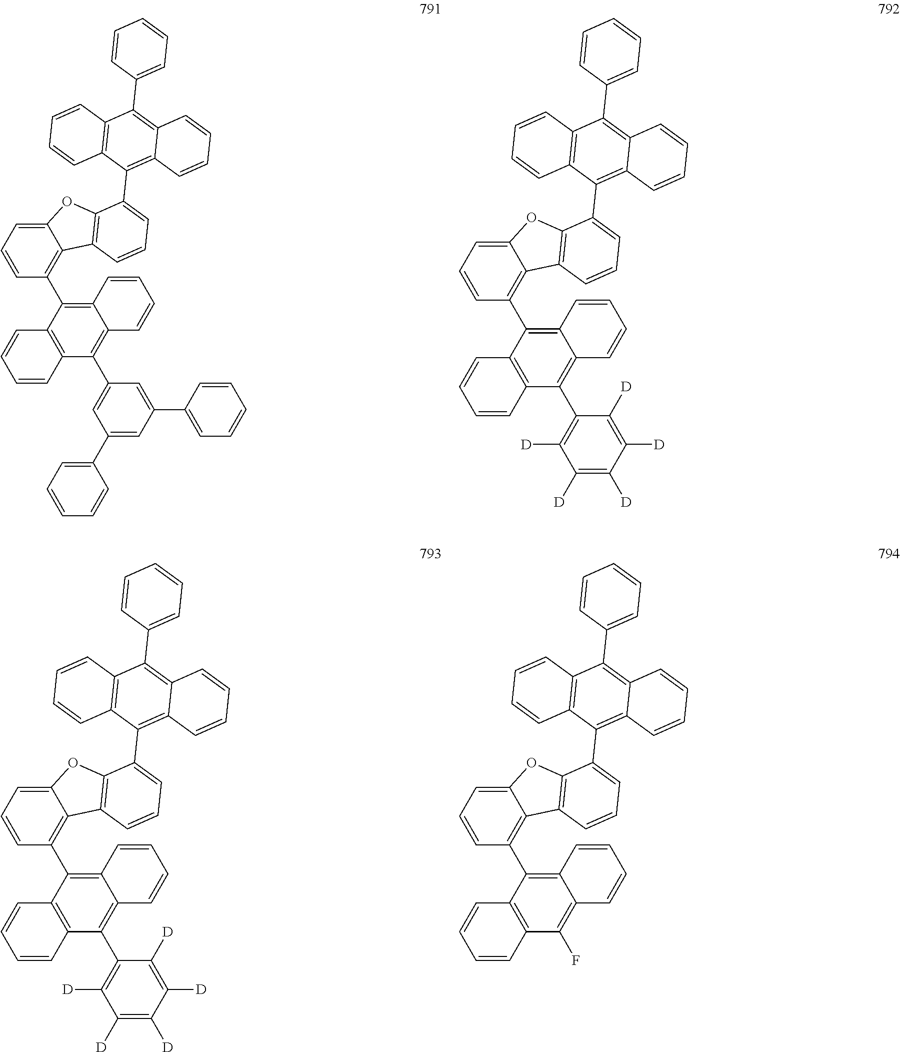 [SMILES: c1ccc(-c2cc(-c3ccccc3)cc(-c3c4ccccc4c(-c4cccc5oc6c(-c7c8ccccc8c(-c8ccccc8)c8ccccc78)cccc6c45)c4ccccc34)c2)cc1 ; [2H]c1c([2H])c([2H])c(-c2c3ccccc3c(-c3cccc4oc5c(-c6c7ccccc7c(-c7ccccc7)c7ccccc67)cccc5c34)c3ccccc23)c([2H])c1[2H] ; [2H]c1c([2H])c([2H])c(-c2c3ccccc3c(-c3cccc4oc5c(-c6c7ccccc7c(-c7ccccc7)c7ccccc67)cccc5c34)c3ccccc23)c([2H])c1[2H] ; Fc1c2ccccc2c(-c2cccc3oc4c(-c5c6ccccc6c(-c6ccccc6)c6ccccc56)cccc4c23)c2ccccc12]

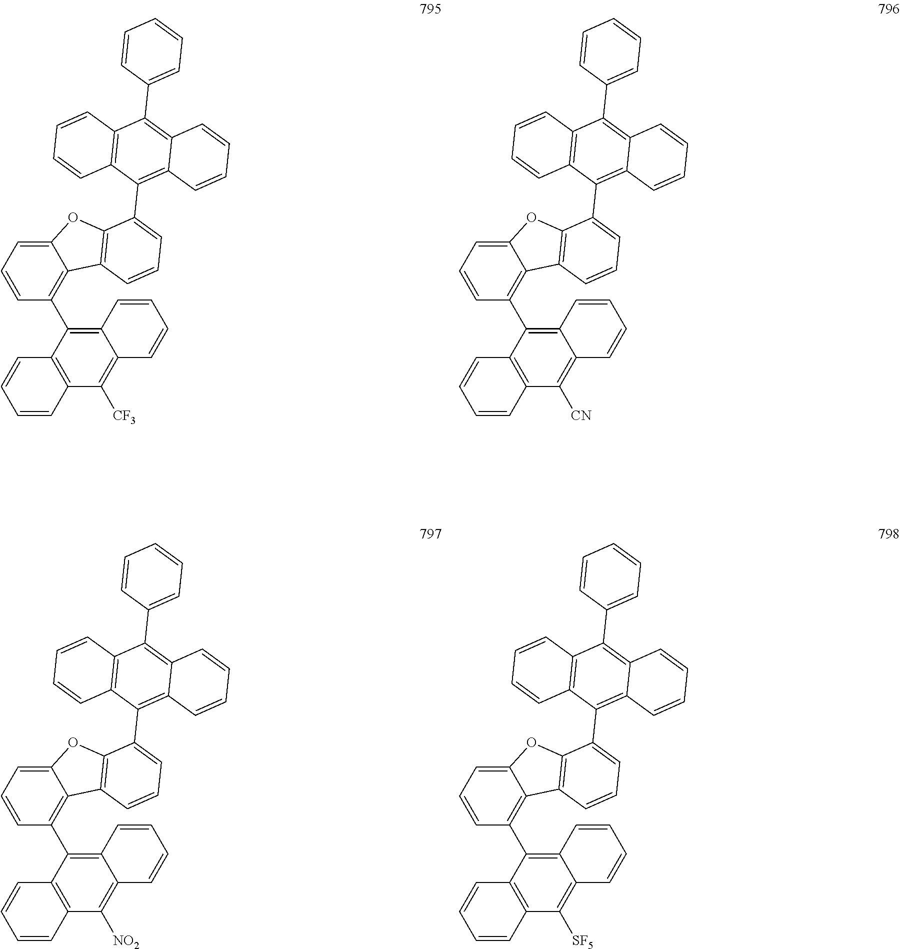

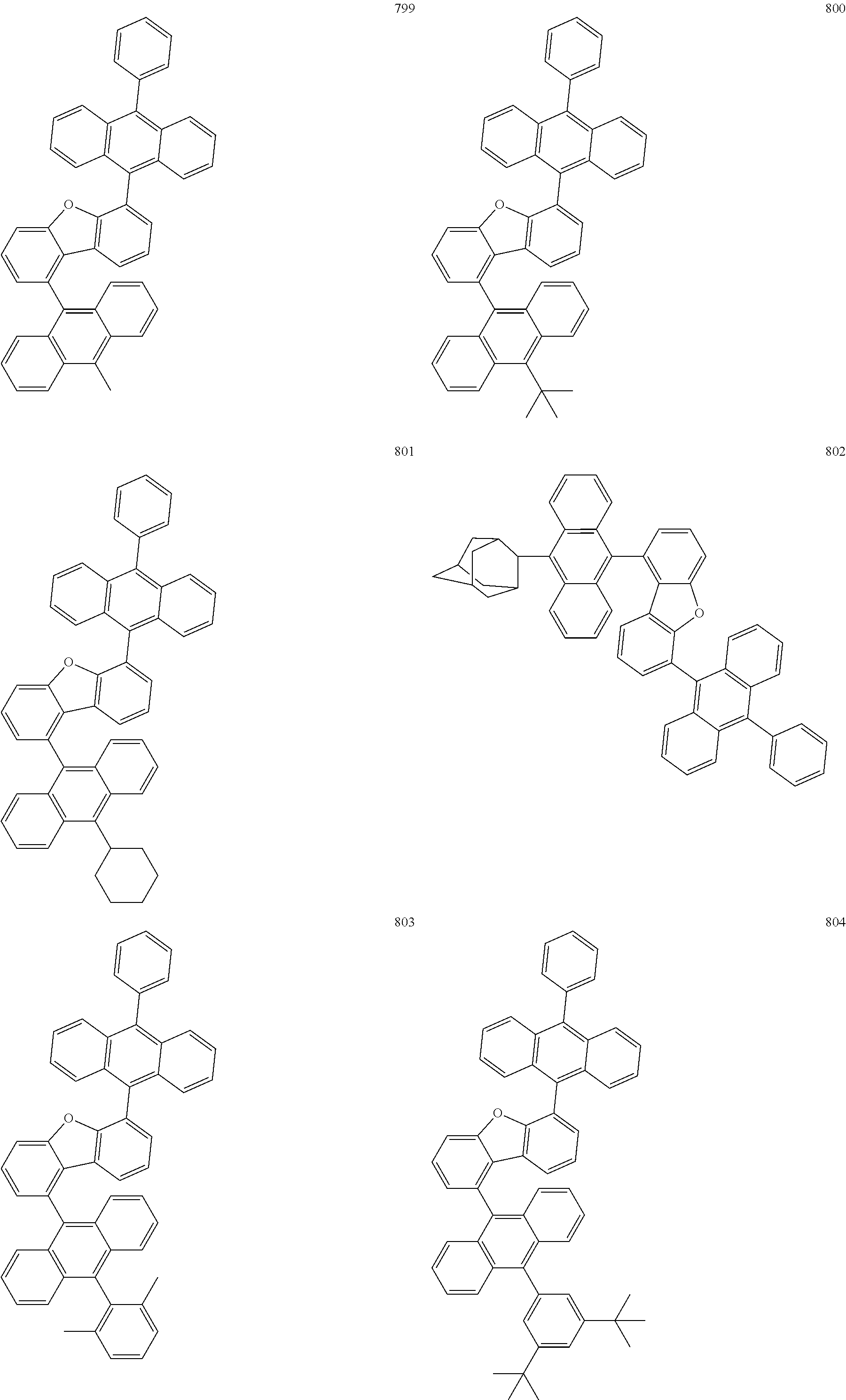

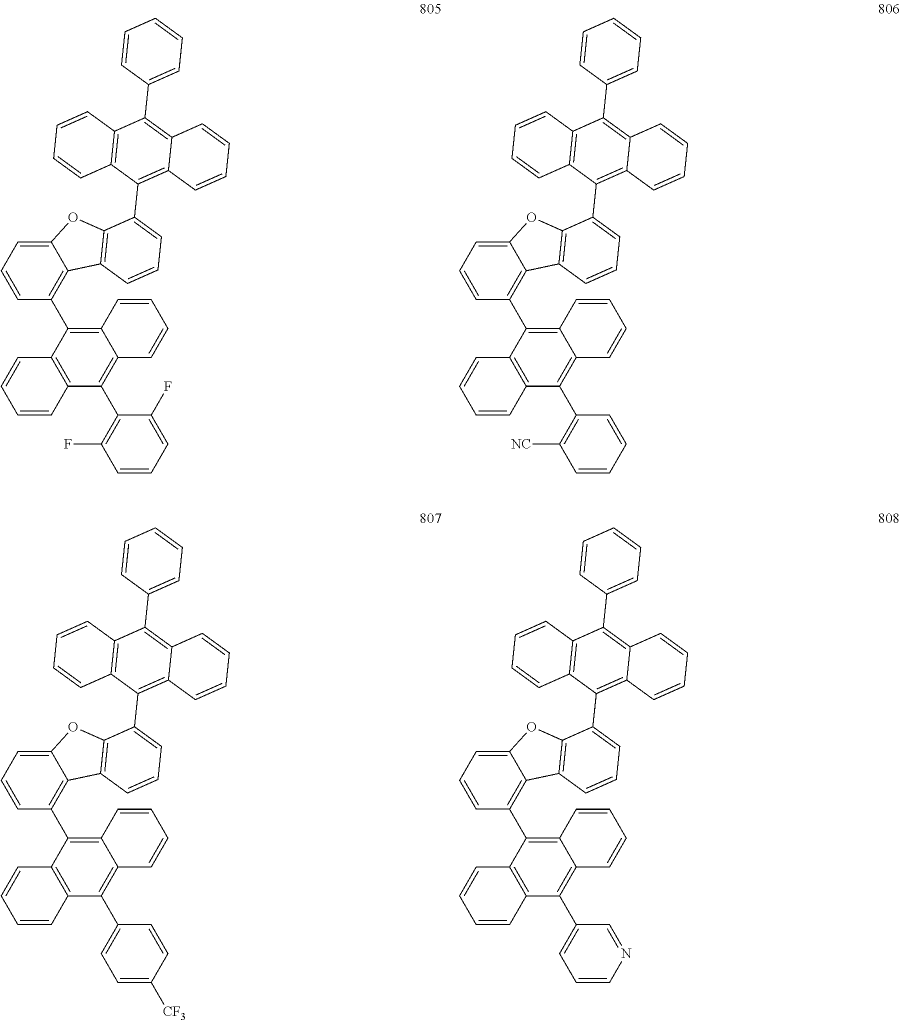 [SMILES: Fc1cccc(F)c1-c1c2ccccc2c(-c2cccc3oc4c(-c5c6ccccc6c(-c6ccccc6)c6ccccc56)cccc4c23)c2ccccc12 ; N#Cc1ccccc1-c1c2ccccc2c(-c2cccc3oc4c(-c5c6ccccc6c(-c6ccccc6)c6ccccc56)cccc4c23)c2ccccc12 ; FC(F)(F)c1ccc(-c2c3ccccc3c(-c3cccc4oc5c(-c6c7ccccc7c(-c7ccccc7)c7ccccc67)cccc5c34)c3ccccc23)cc1 ; c1ccc(-c2c3ccccc3c(-c3cccc4c3oc3cccc(-c5c6ccccc6c(-c6cccnc6)c6ccccc56)c34)c3ccccc23)cc1]

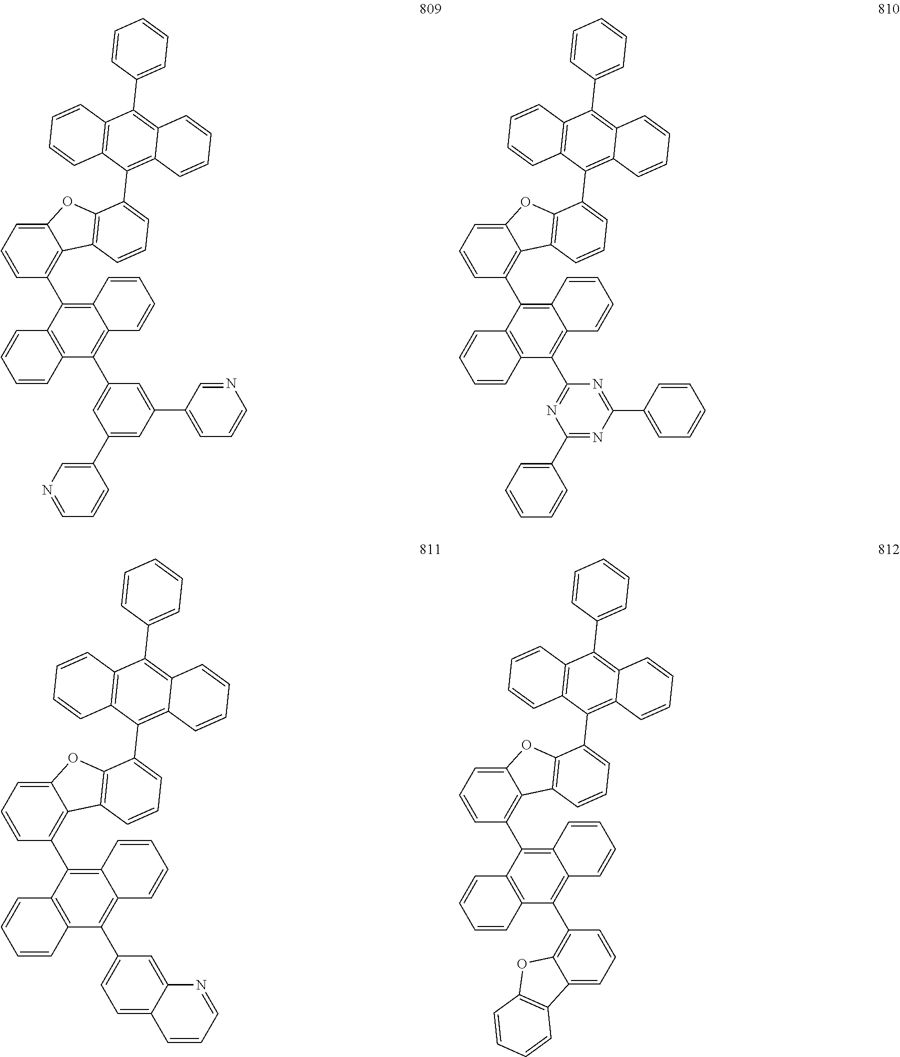 [SMILES: c1ccc(-c2c3ccccc3c(-c3cccc4c3oc3cccc(-c5c6ccccc6c(-c6cc(-c7cccnc7)cc(-c7cccnc7)c6)c6ccccc56)c34)c3ccccc23)cc1 ; c1ccc(-c2nc(-c3ccccc3)nc(-c3c4ccccc4c(-c4cccc5oc6c(-c7c8ccccc8c(-c8ccccc8)c8ccccc78)cccc6c45)c4ccccc34)n2)cc1 ; c1ccc(-c2c3ccccc3c(-c3cccc4c3oc3cccc(-c5c6ccccc6c(-c6ccc7cccnc7c6)c6ccccc56)c34)c3ccccc23)cc1 ; c1ccc(-c2c3ccccc3c(-c3cccc4c3oc3cccc(-c5c6ccccc6c(-c6cccc7c6oc6ccccc67)c6ccccc56)c34)c3ccccc23)cc1]

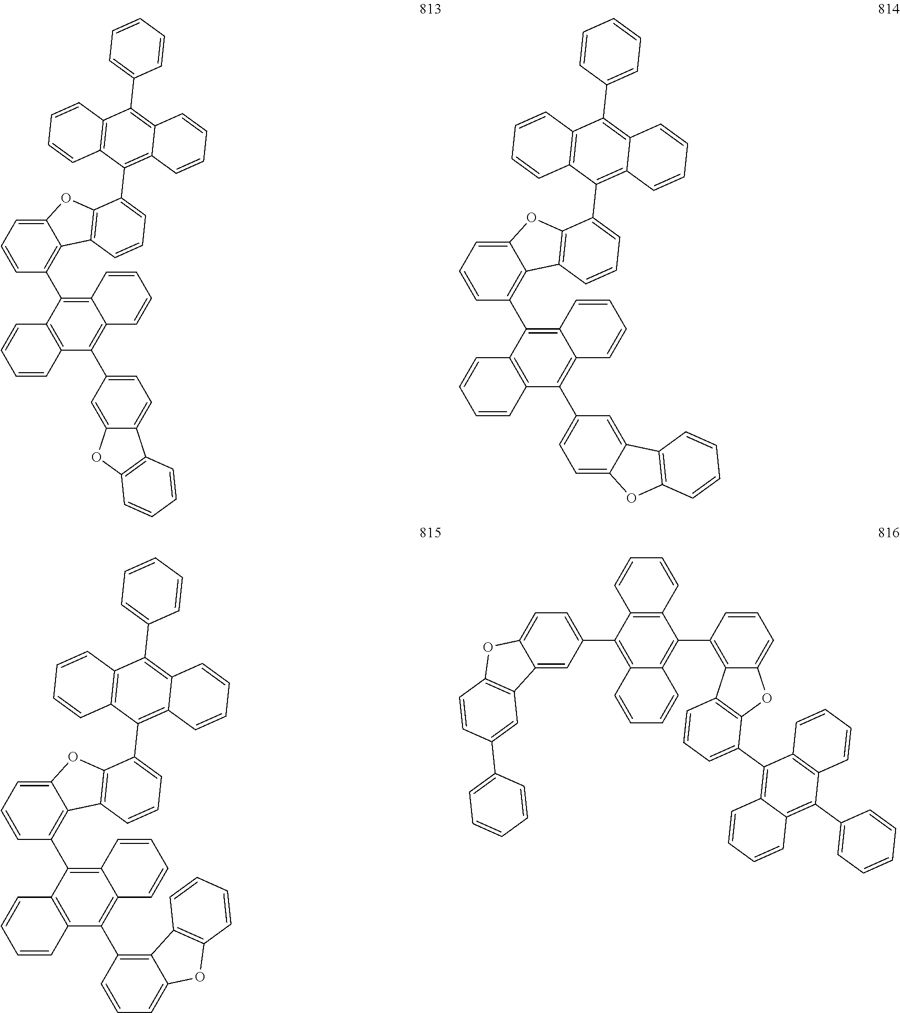

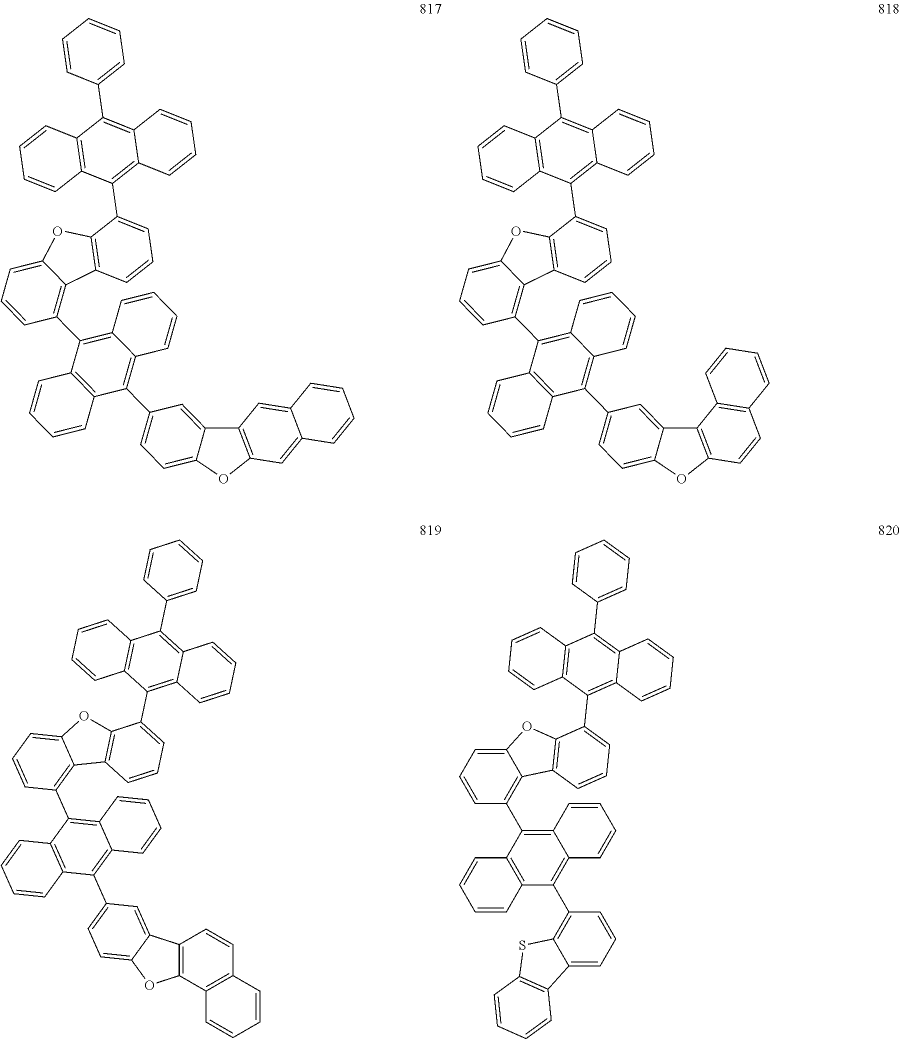 [SMILES: c1ccc(-c2c3ccccc3c(-c3cccc4c3oc3cccc(-c5c6ccccc6c(-c6ccc7oc8cc9ccccc9cc8c7c6)c6ccccc56)c34)c3ccccc23)cc1 ; c1ccc(-c2c3ccccc3c(-c3cccc4c3oc3cccc(-c5c6ccccc6c(-c6ccc7oc8ccc9ccccc9c8c7c6)c6ccccc56)c34)c3ccccc23)cc1 ; c1ccc(-c2c3ccccc3c(-c3cccc4c3oc3cccc(-c5c6ccccc6c(-c6ccc7oc8c9ccccc9ccc8c7c6)c6ccccc56)c34)c3ccccc23)cc1 ; c1ccc(-c2c3ccccc3c(-c3cccc4c3oc3cccc(-c5c6ccccc6c(-c6cccc7c6sc6ccccc67)c6ccccc56)c34)c3ccccc23)cc1]

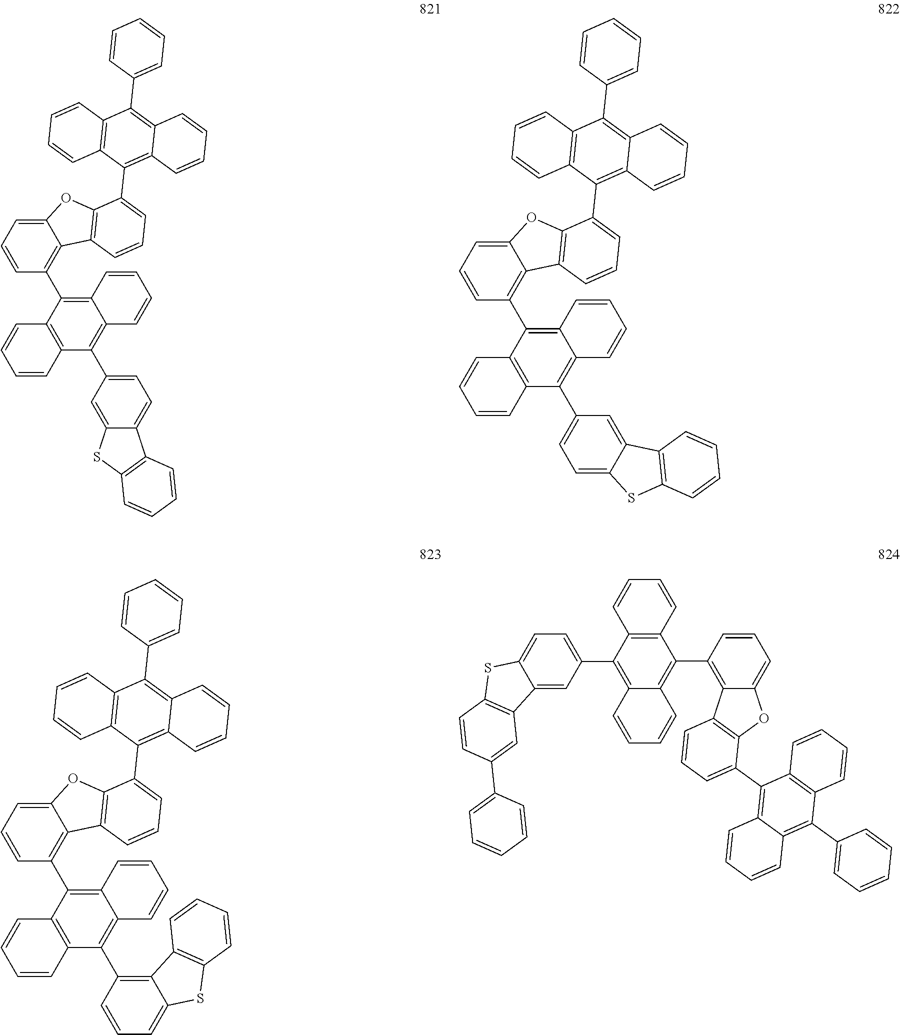 [SMILES: c1ccc(-c2c3ccccc3c(-c3cccc4c3oc3cccc(-c5c6ccccc6c(-c6ccc7c(c6)sc6ccccc67)c6ccccc56)c34)c3ccccc23)cc1 ; c1ccc(-c2c3ccccc3c(-c3cccc4c3oc3cccc(-c5c6ccccc6c(-c6ccc7sc8ccccc8c7c6)c6ccccc56)c34)c3ccccc23)cc1 ; c1ccc(-c2c3ccccc3c(-c3cccc4c3oc3cccc(-c5c6ccccc6c(-c6cccc7sc8ccccc8c67)c6ccccc56)c34)c3ccccc23)cc1 ; c1ccc(-c2ccc3sc4ccc(-c5c6ccccc6c(-c6cccc7oc8c(-c9c%10ccccc%10c(-c%10ccccc%10)c%10ccccc9%10)cccc8c67)c6ccccc56)cc4c3c2)cc1]

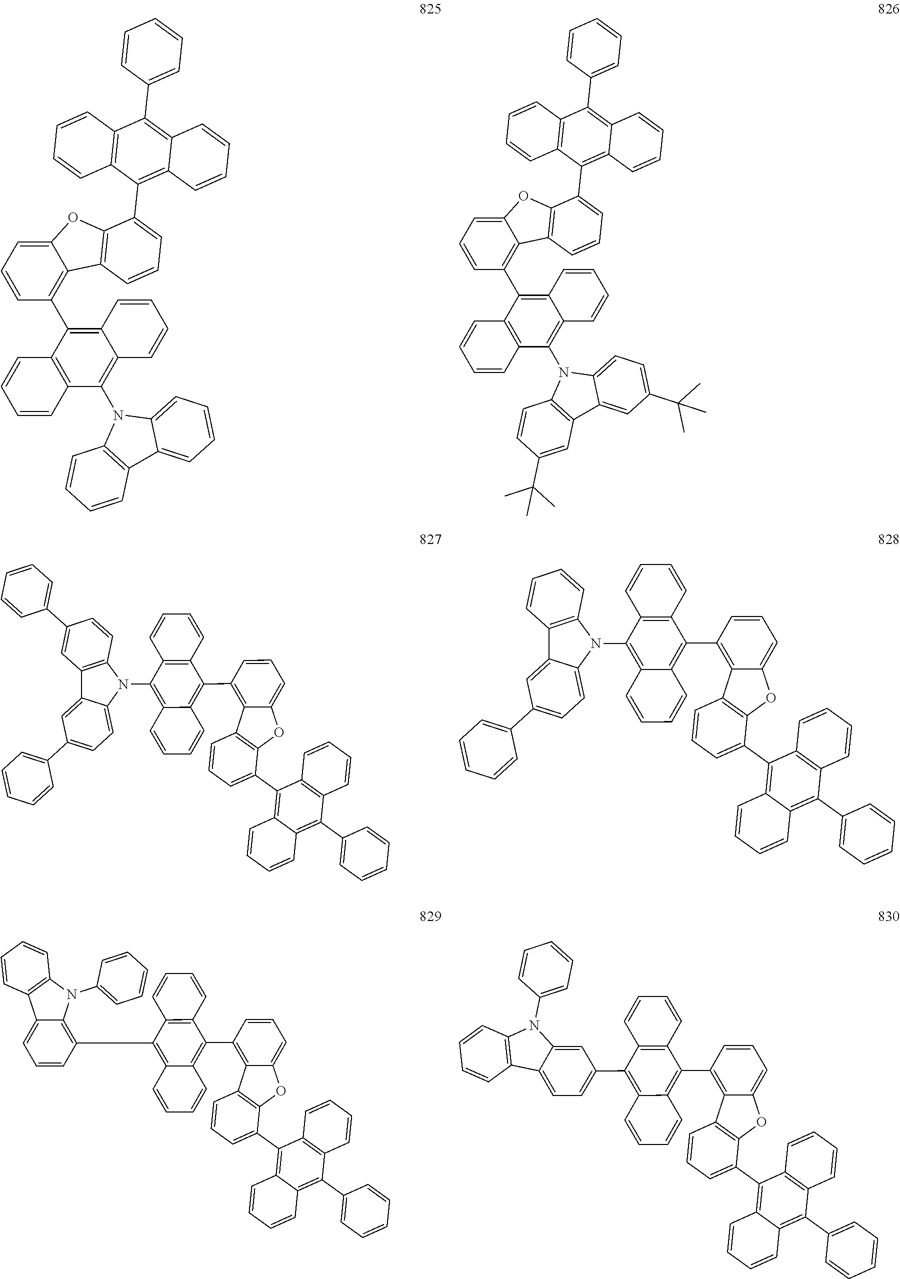

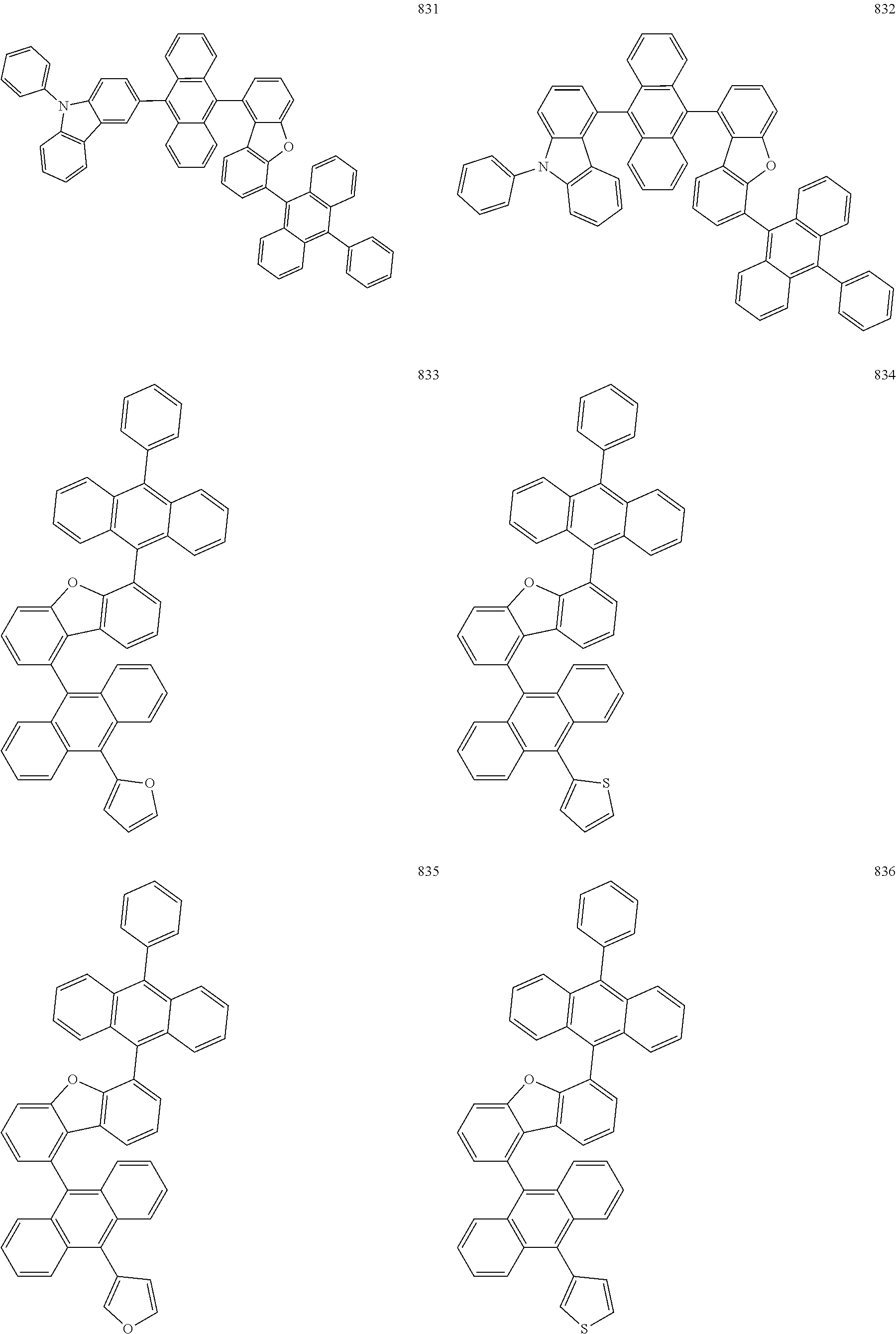

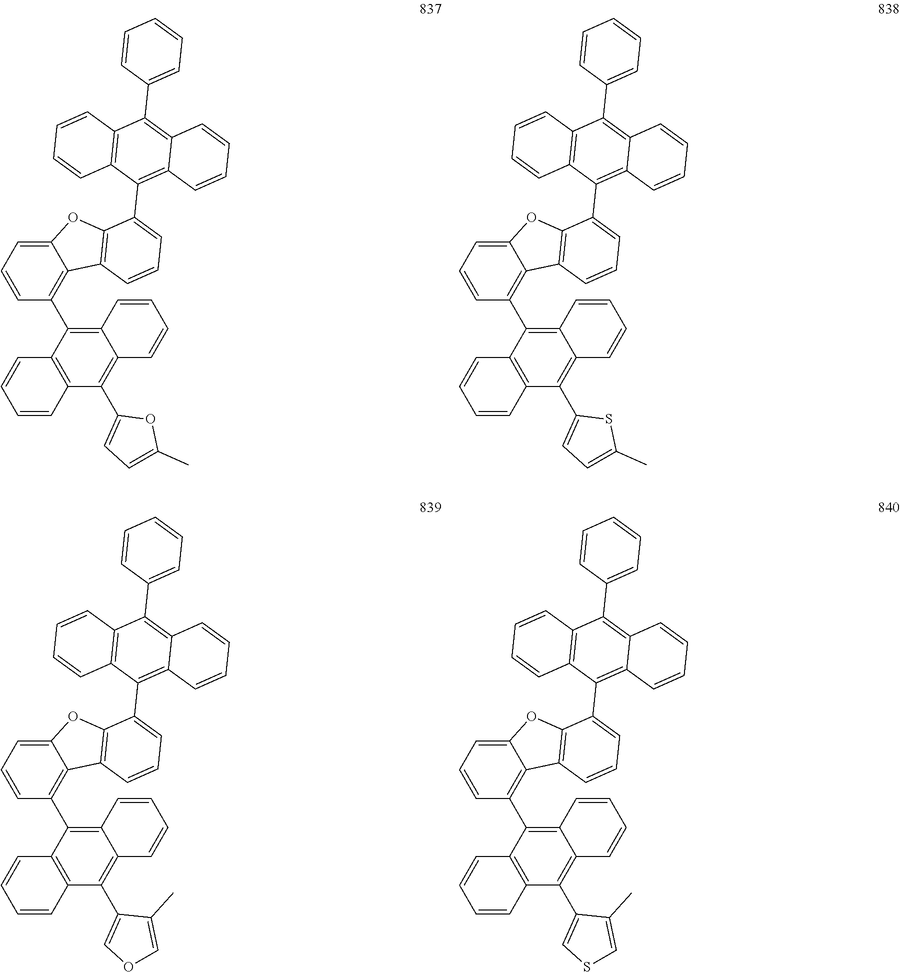 [SMILES: Cc1ccc(-c2c3ccccc3c(-c3cccc4oc5c(-c6c7ccccc7c(-c7ccccc7)c7ccccc67)cccc5c34)c3ccccc23)o1 ; Cc1ccc(-c2c3ccccc3c(-c3cccc4oc5c(-c6c7ccccc7c(-c7ccccc7)c7ccccc67)cccc5c34)c3ccccc23)s1 ; Cc1cocc1-c1c2ccccc2c(-c2cccc3oc4c(-c5c6ccccc6c(-c6ccccc6)c6ccccc56)cccc4c23)c2ccccc12 ; Cc1cscc1-c1c2ccccc2c(-c2cccc3oc4c(-c5c6ccccc6c(-c6ccccc6)c6ccccc56)cccc4c23)c2ccccc12]

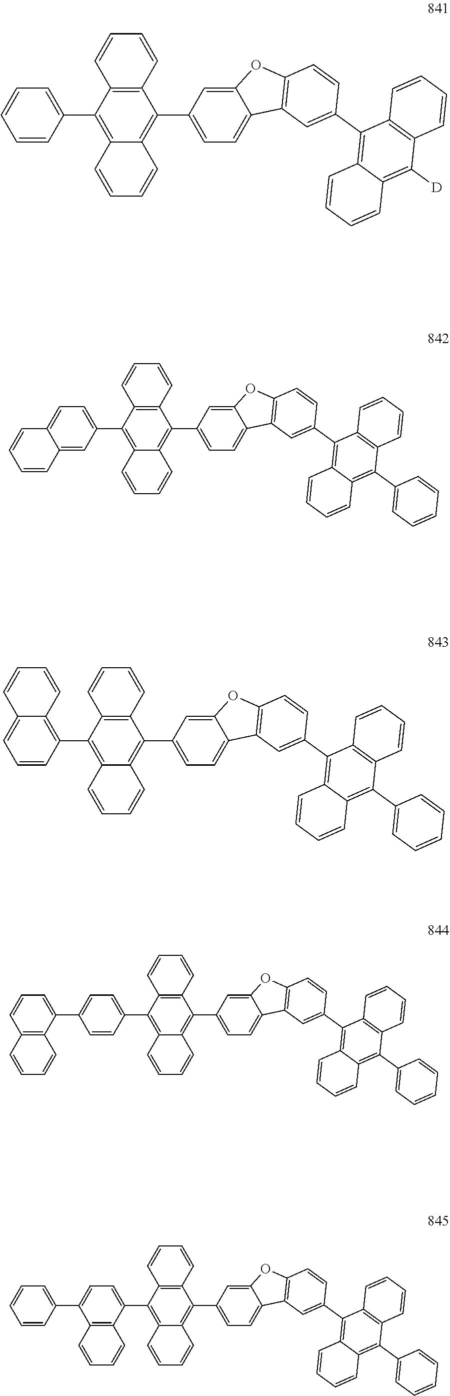 [SMILES: [2H]c1c2ccccc2c(-c2ccc3oc4cc(-c5c6ccccc6c(-c6ccccc6)c6ccccc56)ccc4c3c2)c2ccccc12 ; c1ccc(-c2c3ccccc3c(-c3ccc4oc5cc(-c6c7ccccc7c(-c7ccc8ccccc8c7)c7ccccc67)ccc5c4c3)c3ccccc23)cc1 ; c1ccc(-c2c3ccccc3c(-c3ccc4oc5cc(-c6c7ccccc7c(-c7cccc8ccccc78)c7ccccc67)ccc5c4c3)c3ccccc23)cc1 ; c1ccc(-c2c3ccccc3c(-c3ccc4oc5cc(-c6c7ccccc7c(-c7ccc(-c8cccc9ccccc89)cc7)c7ccccc67)ccc5c4c3)c3ccccc23)cc1 ; c1ccc(-c2ccc(-c3c4ccccc4c(-c4ccc5c(c4)oc4ccc(-c6c7ccccc7c(-c7ccccc7)c7ccccc67)cc45)c4ccccc34)c3ccccc23)cc1]

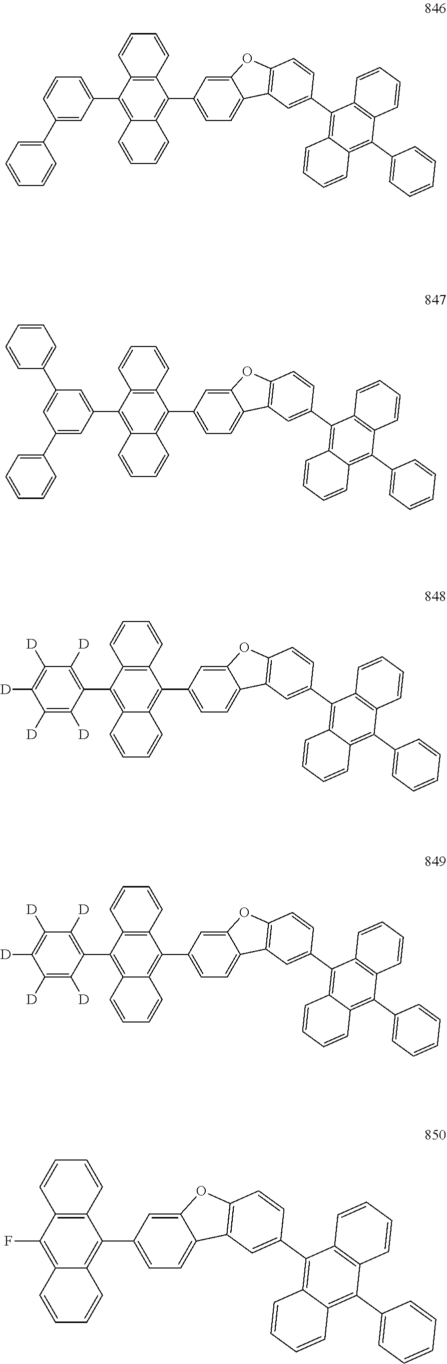

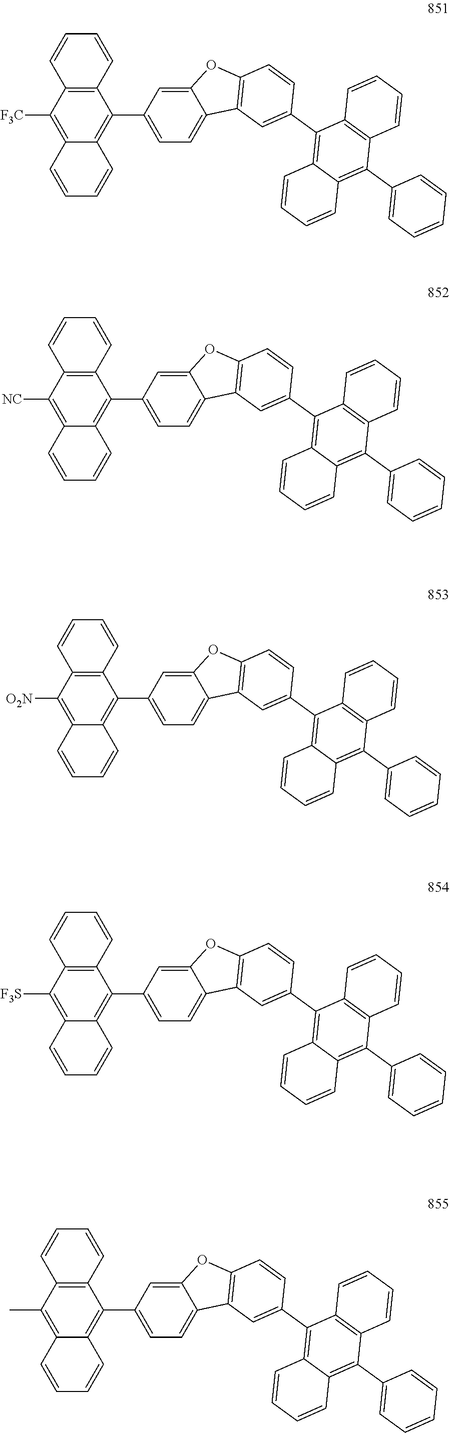

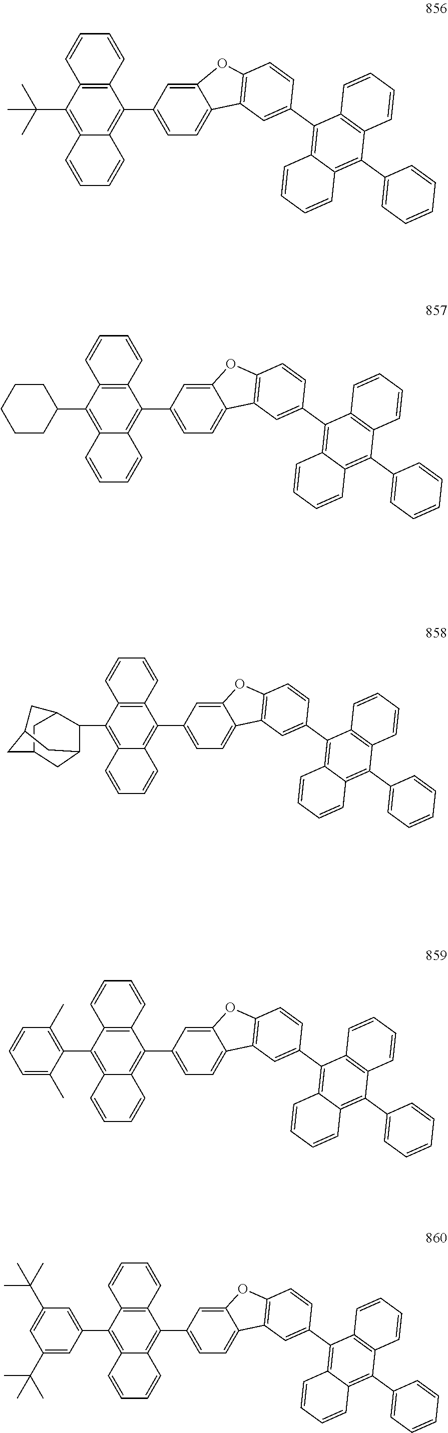 [SMILES: CC(C)(C)c1c2ccccc2c(-c2ccc3c(c2)oc2ccc(-c4c5ccccc5c(-c5ccccc5)c5ccccc45)cc23)c2ccccc12 ; c1ccc(-c2c3ccccc3c(-c3ccc4oc5cc(-c6c7ccccc7c(C7CCCCC7)c7ccccc67)ccc5c4c3)c3ccccc23)cc1 ; c1ccc(-c2c3ccccc3c(-c3ccc4oc5cc(-c6c7ccccc7c(C7C8CC9CC(C8)CC7C9)c7ccccc67)ccc5c4c3)c3ccccc23)cc1 ; Cc1cccc(C)c1-c1c2ccccc2c(-c2ccc3c(c2)oc2ccc(-c4c5ccccc5c(-c5ccccc5)c5ccccc45)cc23)c2ccccc12 ; CC(C)(C)c1cc(-c2c3ccccc3c(-c3ccc4c(c3)oc3ccc(-c5c6ccccc6c(-c6ccccc6)c6ccccc56)cc34)c3ccccc23)cc(C(C)(C)C)c1]

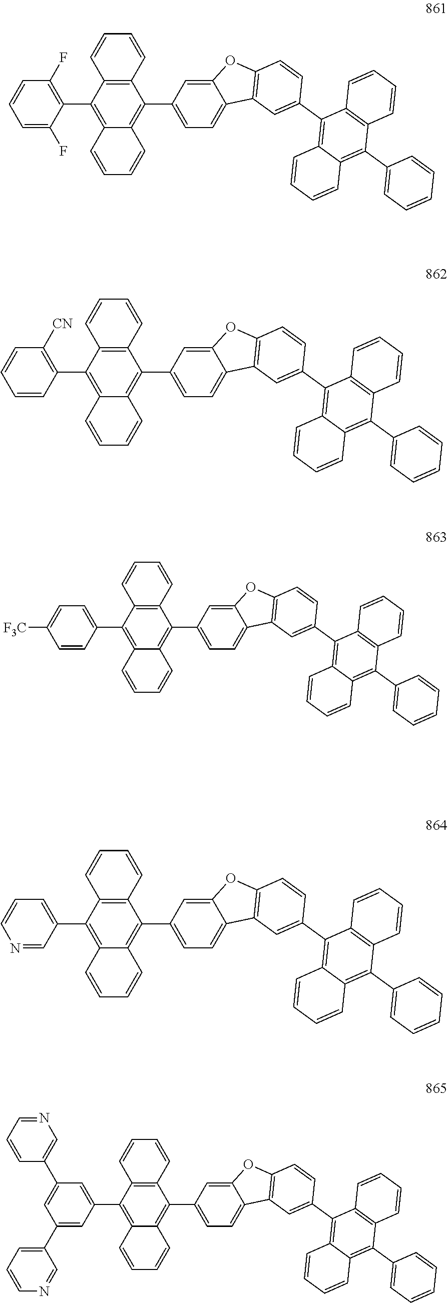 [SMILES: Fc1cccc(F)c1-c1c2ccccc2c(-c2ccc3c(c2)oc2ccc(-c4c5ccccc5c(-c5ccccc5)c5ccccc45)cc23)c2ccccc12 ; N#Cc1ccccc1-c1c2ccccc2c(-c2ccc3c(c2)oc2ccc(-c4c5ccccc5c(-c5ccccc5)c5ccccc45)cc23)c2ccccc12 ; FC(F)(F)c1ccc(-c2c3ccccc3c(-c3ccc4c(c3)oc3ccc(-c5c6ccccc6c(-c6ccccc6)c6ccccc56)cc34)c3ccccc23)cc1 ; c1ccc(-c2c3ccccc3c(-c3ccc4oc5cc(-c6c7ccccc7c(-c7cccnc7)c7ccccc67)ccc5c4c3)c3ccccc23)cc1 ; c1ccc(-c2c3ccccc3c(-c3ccc4oc5cc(-c6c7ccccc7c(-c7cc(-c8cccnc8)cc(-c8cccnc8)c7)c7ccccc67)ccc5c4c3)c3ccccc23)cc1]

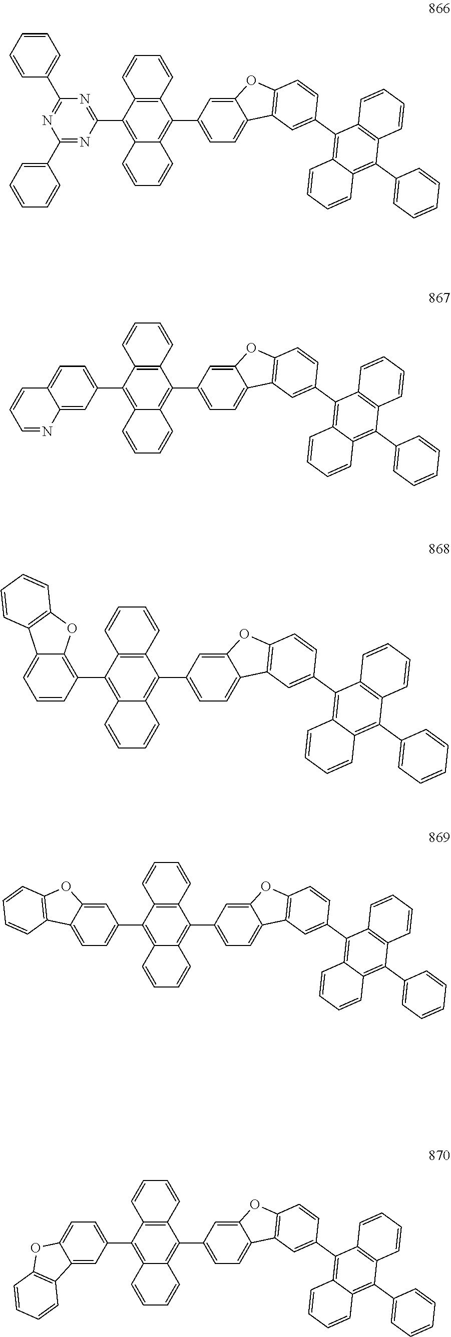

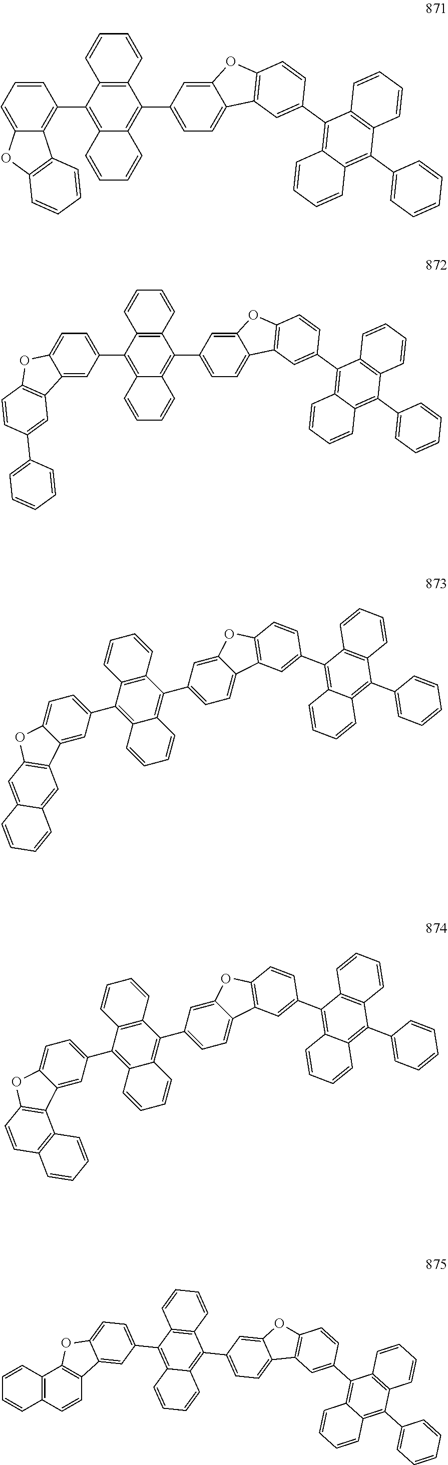

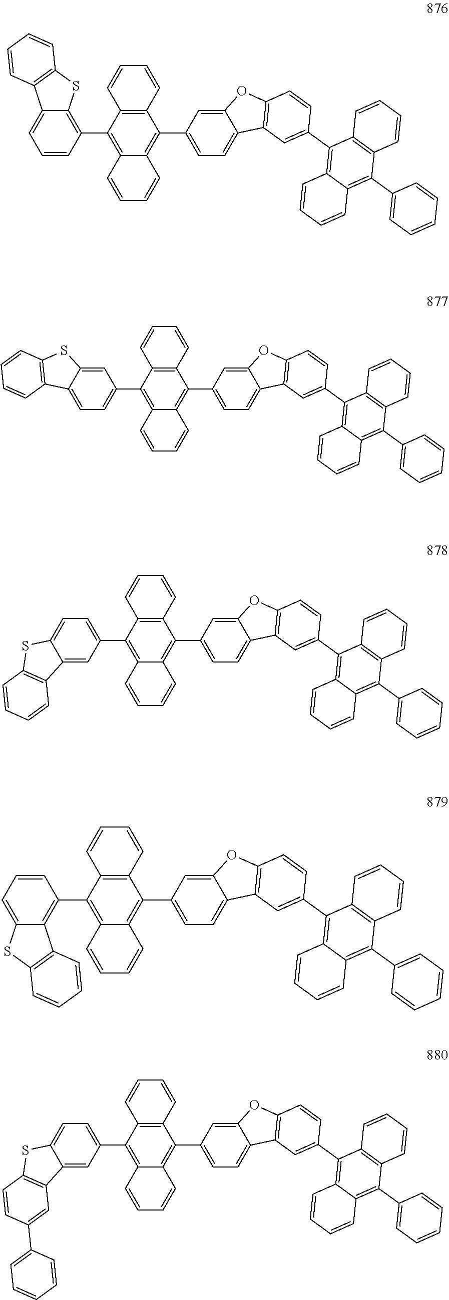

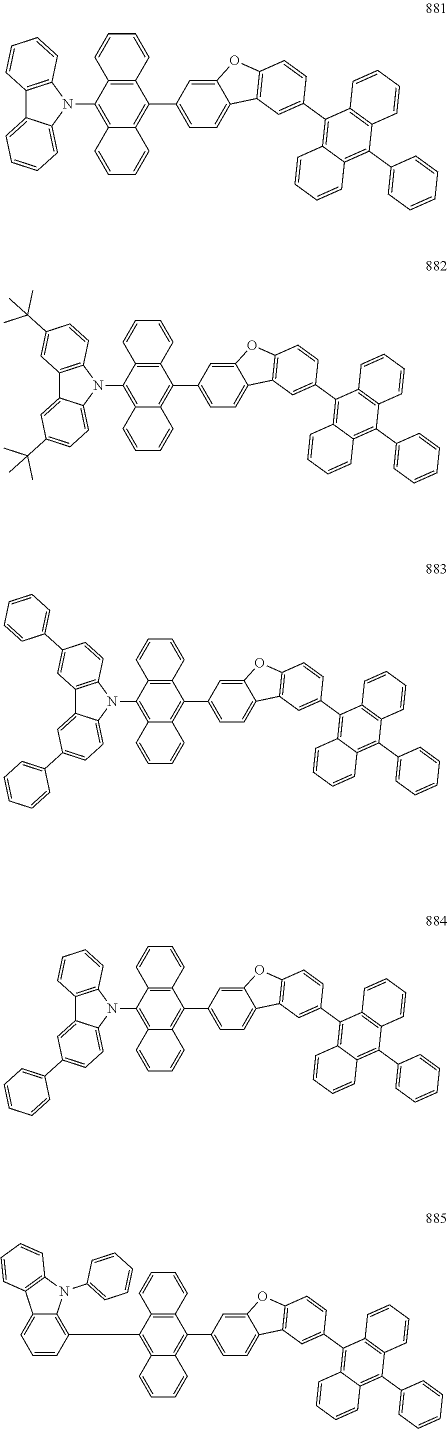

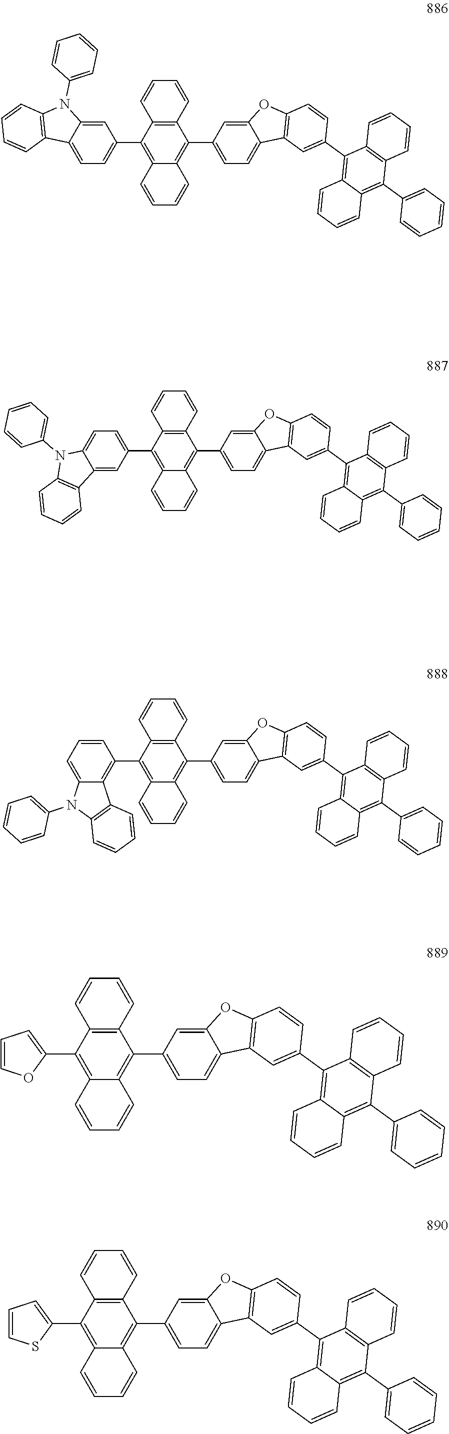

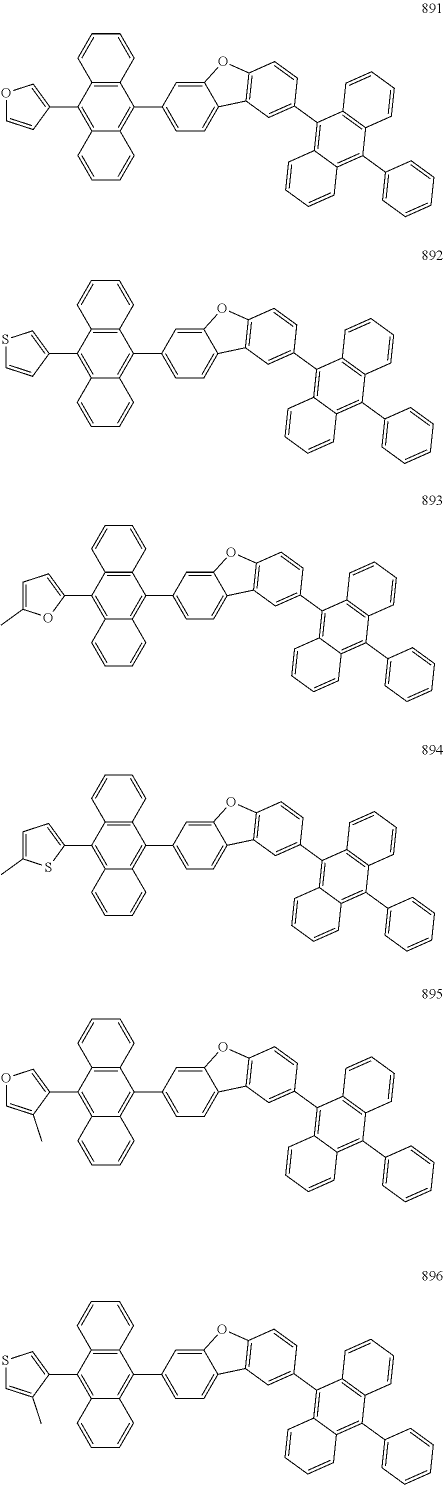

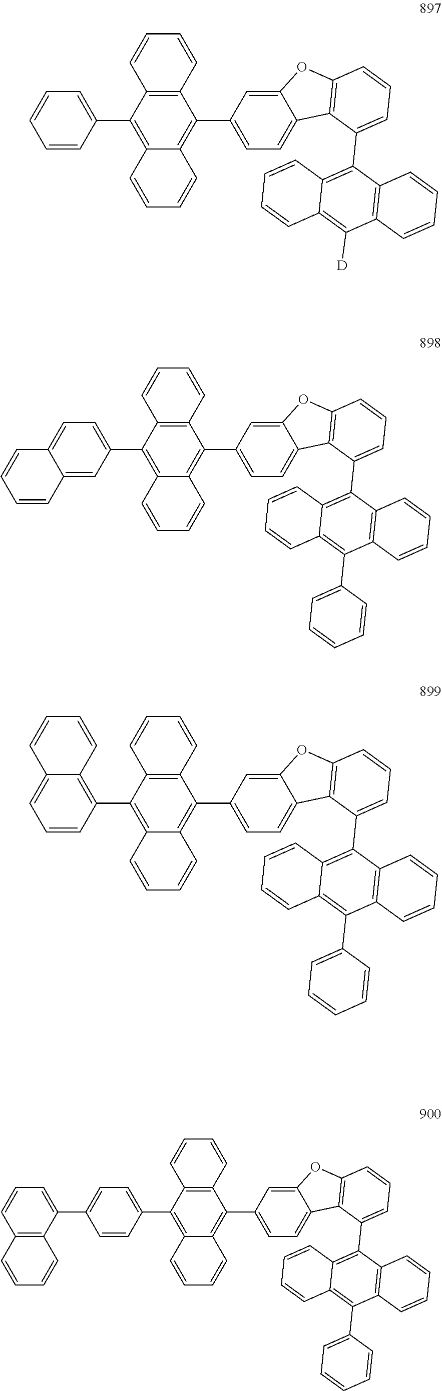 [SMILES: [2H]c1c2ccccc2c(-c2cccc3oc4cc(-c5c6ccccc6c(-c6ccccc6)c6ccccc56)ccc4c23)c2ccccc12 ; c1ccc(-c2c3ccccc3c(-c3cccc4oc5cc(-c6c7ccccc7c(-c7ccc8ccccc8c7)c7ccccc67)ccc5c34)c3ccccc23)cc1 ; c1ccc(-c2c3ccccc3c(-c3cccc4oc5cc(-c6c7ccccc7c(-c7cccc8ccccc78)c7ccccc67)ccc5c34)c3ccccc23)cc1 ; c1ccc(-c2c3ccccc3c(-c3cccc4oc5cc(-c6c7ccccc7c(-c7ccc(-c8cccc9ccccc89)cc7)c7ccccc67)ccc5c34)c3ccccc23)cc1]

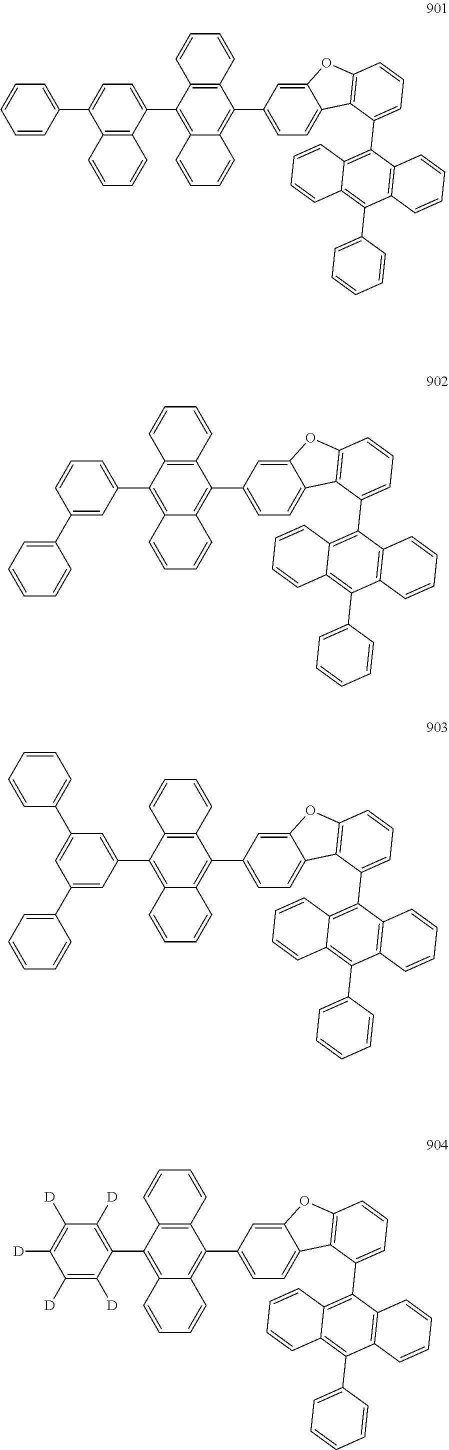 [SMILES: c1ccc(-c2ccc(-c3c4ccccc4c(-c4ccc5c(c4)oc4cccc(-c6c7ccccc7c(-c7ccccc7)c7ccccc67)c45)c4ccccc34)c3ccccc23)cc1 ; c1ccc(-c2cccc(-c3c4ccccc4c(-c4ccc5c(c4)oc4cccc(-c6c7ccccc7c(-c7ccccc7)c7ccccc67)c45)c4ccccc34)c2)cc1 ; c1ccc(-c2cc(-c3ccccc3)cc(-c3c4ccccc4c(-c4ccc5c(c4)oc4cccc(-c6c7ccccc7c(-c7ccccc7)c7ccccc67)c45)c4ccccc34)c2)cc1 ; [2H]c1c([2H])c([2H])c(-c2c3ccccc3c(-c3ccc4c(c3)oc3cccc(-c5c6ccccc6c(-c6ccccc6)c6ccccc56)c34)c3ccccc23)c([2H])c1[2H]]

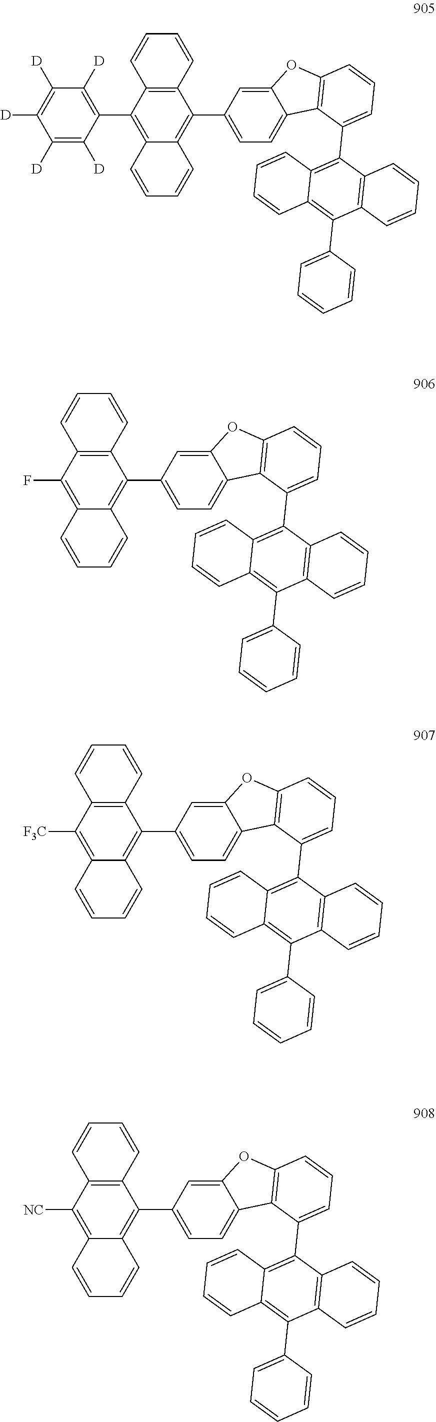 [SMILES: [2H]c1c([2H])c([2H])c(-c2c3ccccc3c(-c3ccc4c(c3)oc3cccc(-c5c6ccccc6c(-c6ccccc6)c6ccccc56)c34)c3ccccc23)c([2H])c1[2H] ; Fc1c2ccccc2c(-c2ccc3c(c2)oc2cccc(-c4c5ccccc5c(-c5ccccc5)c5ccccc45)c23)c2ccccc12 ; FC(F)(F)c1c2ccccc2c(-c2ccc3c(c2)oc2cccc(-c4c5ccccc5c(-c5ccccc5)c5ccccc45)c23)c2ccccc12 ; N#Cc1c2ccccc2c(-c2ccc3c(c2)oc2cccc(-c4c5ccccc5c(-c5ccccc5)c5ccccc45)c23)c2ccccc12]

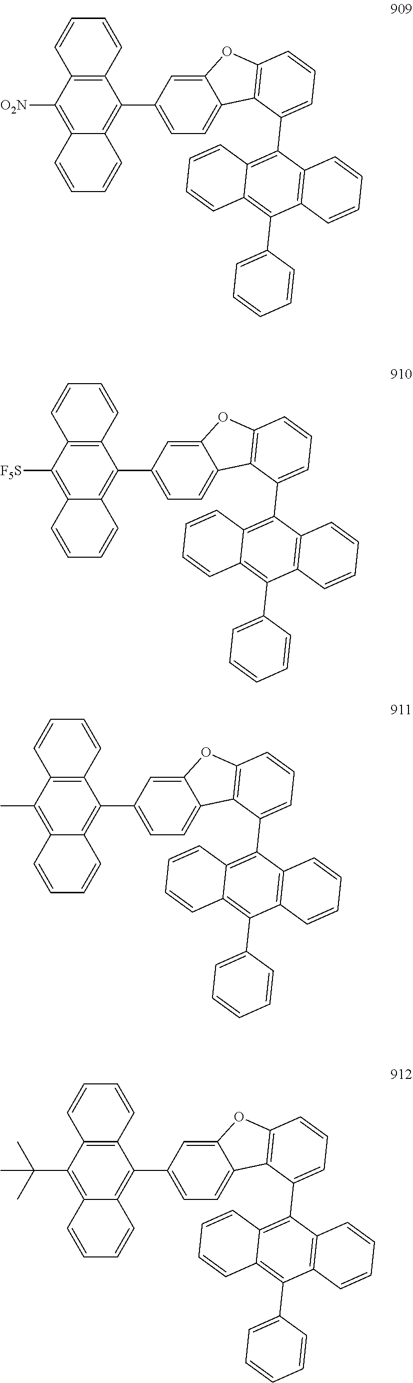

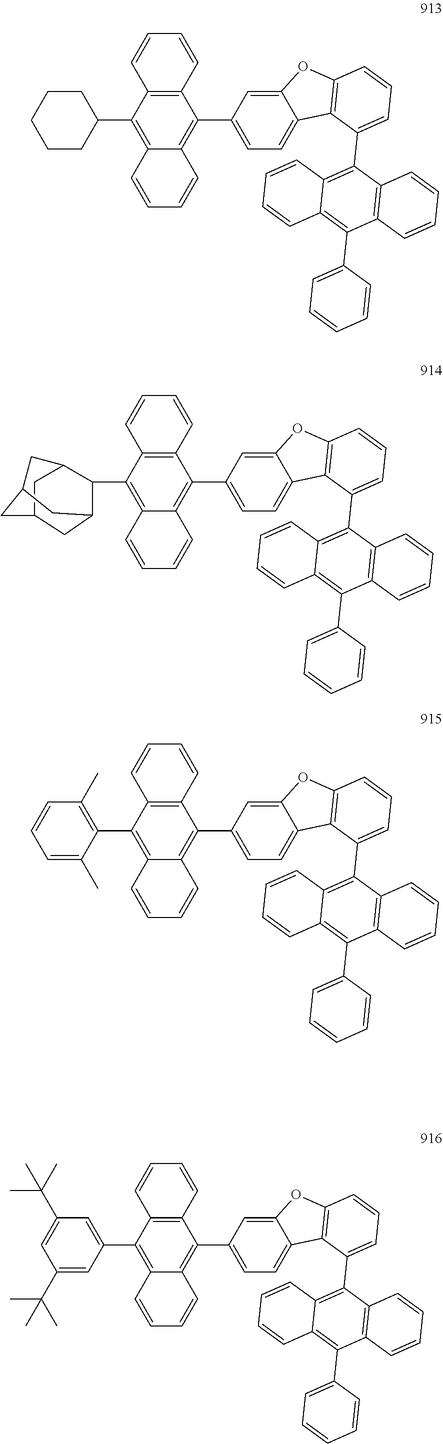 [SMILES: c1ccc(-c2c3ccccc3c(-c3cccc4oc5cc(-c6c7ccccc7c(C7CCCCC7)c7ccccc67)ccc5c34)c3ccccc23)cc1 ; c1ccc(-c2c3ccccc3c(-c3cccc4oc5cc(-c6c7ccccc7c(C7C8CC9CC(C8)CC7C9)c7ccccc67)ccc5c34)c3ccccc23)cc1 ; Cc1cccc(C)c1-c1c2ccccc2c(-c2ccc3c(c2)oc2cccc(-c4c5ccccc5c(-c5ccccc5)c5ccccc45)c23)c2ccccc12 ; CC(C)(C)c1cc(-c2c3ccccc3c(-c3ccc4c(c3)oc3cccc(-c5c6ccccc6c(-c6ccccc6)c6ccccc56)c34)c3ccccc23)cc(C(C)(C)C)c1]

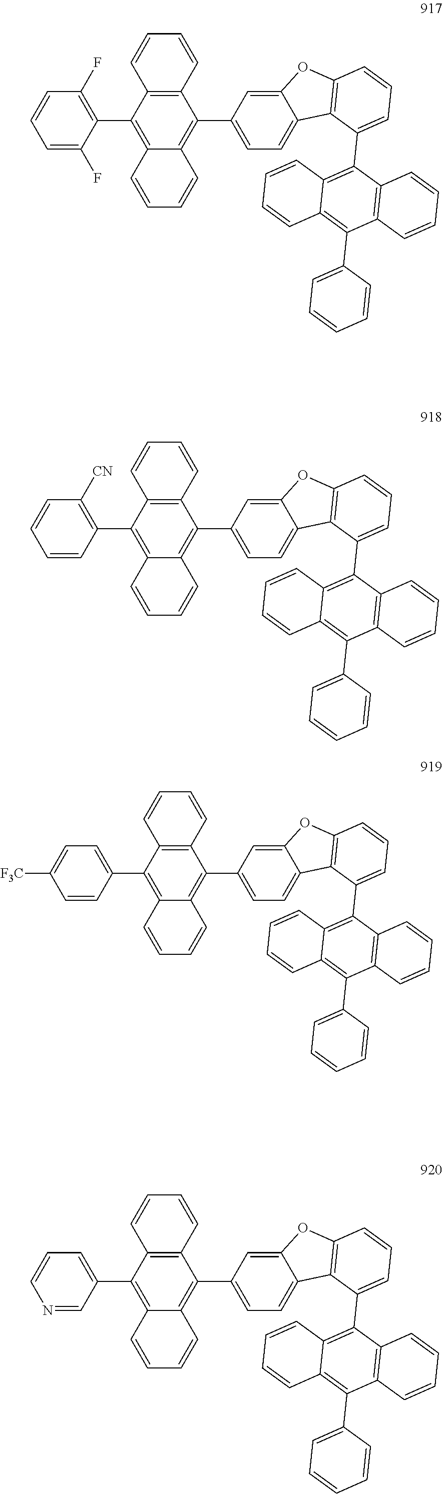 [SMILES: Fc1cccc(F)c1-c1c2ccccc2c(-c2ccc3c(c2)oc2cccc(-c4c5ccccc5c(-c5ccccc5)c5ccccc45)c23)c2ccccc12 ; N#Cc1ccccc1-c1c2ccccc2c(-c2ccc3c(c2)oc2cccc(-c4c5ccccc5c(-c5ccccc5)c5ccccc45)c23)c2ccccc12 ; FC(F)(F)c1ccc(-c2c3ccccc3c(-c3ccc4c(c3)oc3cccc(-c5c6ccccc6c(-c6ccccc6)c6ccccc56)c34)c3ccccc23)cc1 ; c1ccc(-c2c3ccccc3c(-c3cccc4oc5cc(-c6c7ccccc7c(-c7cccnc7)c7ccccc67)ccc5c34)c3ccccc23)cc1]

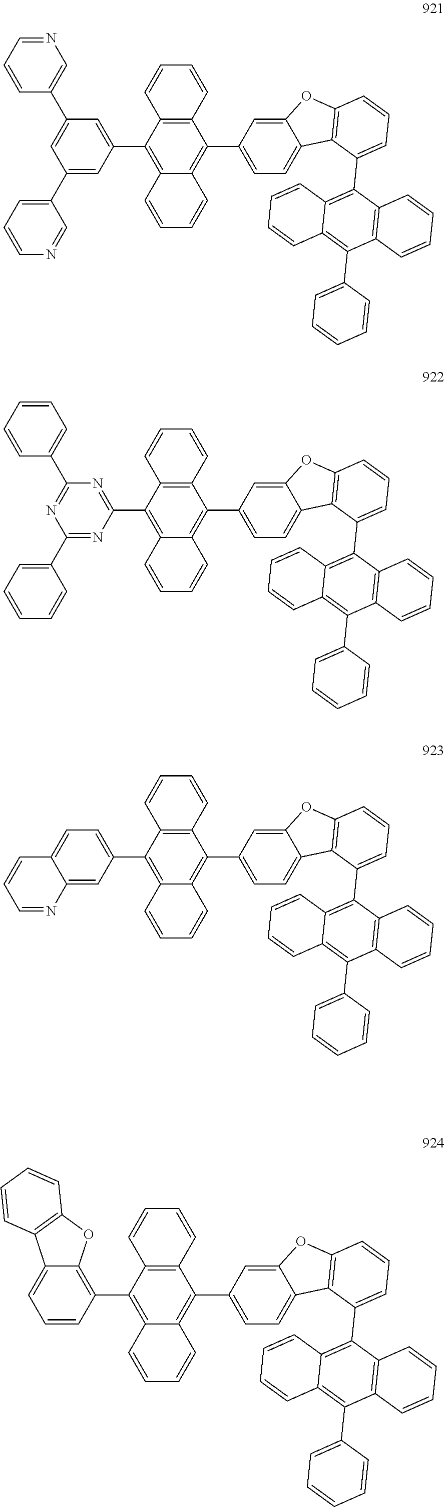 [SMILES: c1ccc(-c2c3ccccc3c(-c3cccc4oc5cc(-c6c7ccccc7c(-c7cc(-c8cccnc8)cc(-c8cccnc8)c7)c7ccccc67)ccc5c34)c3ccccc23)cc1 ; c1ccc(-c2nc(-c3ccccc3)nc(-c3c4ccccc4c(-c4ccc5c(c4)oc4cccc(-c6c7ccccc7c(-c7ccccc7)c7ccccc67)c45)c4ccccc34)n2)cc1 ; c1ccc(-c2c3ccccc3c(-c3cccc4oc5cc(-c6c7ccccc7c(-c7ccc8cccnc8c7)c7ccccc67)ccc5c34)c3ccccc23)cc1 ; c1ccc(-c2c3ccccc3c(-c3cccc4oc5cc(-c6c7ccccc7c(-c7cccc8c7oc7ccccc78)c7ccccc67)ccc5c34)c3ccccc23)cc1]

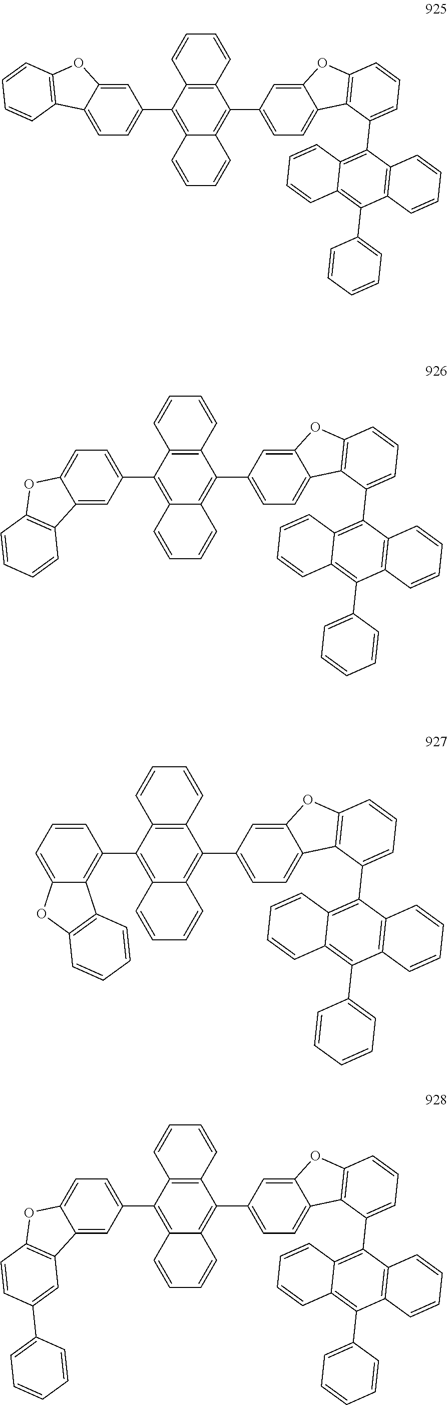 [SMILES: c1ccc(-c2c3ccccc3c(-c3cccc4oc5cc(-c6c7ccccc7c(-c7ccc8c(c7)oc7ccccc78)c7ccccc67)ccc5c34)c3ccccc23)cc1 ; c1ccc(-c2c3ccccc3c(-c3cccc4oc5cc(-c6c7ccccc7c(-c7ccc8oc9ccccc9c8c7)c7ccccc67)ccc5c34)c3ccccc23)cc1 ; c1ccc(-c2c3ccccc3c(-c3cccc4oc5cc(-c6c7ccccc7c(-c7cccc8oc9ccccc9c78)c7ccccc67)ccc5c34)c3ccccc23)cc1 ; c1ccc(-c2ccc3oc4ccc(-c5c6ccccc6c(-c6ccc7c(c6)oc6cccc(-c8c9ccccc9c(-c9ccccc9)c9ccccc89)c67)c6ccccc56)cc4c3c2)cc1]

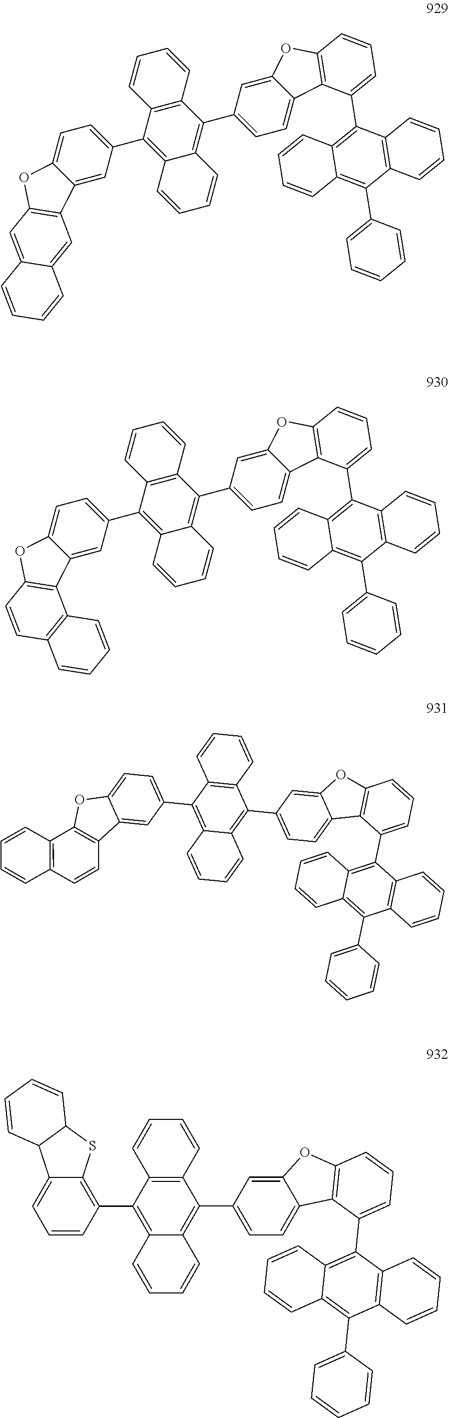 [SMILES: c1ccc(-c2c3ccccc3c(-c3cccc4oc5cc(-c6c7ccccc7c(-c7ccc8oc9cc%10ccccc%10cc9c8c7)c7ccccc67)ccc5c34)c3ccccc23)cc1 ; c1ccc(-c2c3ccccc3c(-c3cccc4oc5cc(-c6c7ccccc7c(-c7ccc8oc9ccc%10ccccc%10c9c8c7)c7ccccc67)ccc5c34)c3ccccc23)cc1 ; c1ccc(-c2c3ccccc3c(-c3cccc4oc5cc(-c6c7ccccc7c(-c7ccc8oc9c%10ccccc%10ccc9c8c7)c7ccccc67)ccc5c34)c3ccccc23)cc1 ; C1=CC2Sc3c(-c4c5ccccc5c(-c5ccc6c(c5)oc5cccc(-c7c8ccccc8c(-c8ccccc8)c8ccccc78)c56)c5ccccc45)cccc3C2C=C1]

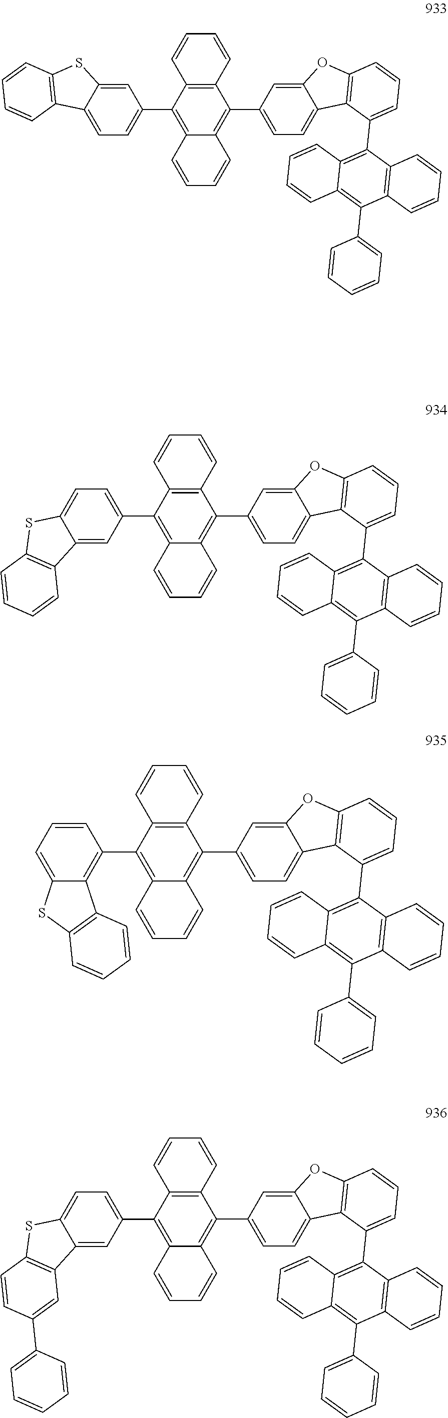 [SMILES: c1ccc(-c2c3ccccc3c(-c3cccc4oc5cc(-c6c7ccccc7c(-c7ccc8c(c7)sc7ccccc78)c7ccccc67)ccc5c34)c3ccccc23)cc1 ; c1ccc(-c2c3ccccc3c(-c3cccc4oc5cc(-c6c7ccccc7c(-c7ccc8sc9ccccc9c8c7)c7ccccc67)ccc5c34)c3ccccc23)cc1 ; c1ccc(-c2c3ccccc3c(-c3cccc4oc5cc(-c6c7ccccc7c(-c7cccc8sc9ccccc9c78)c7ccccc67)ccc5c34)c3ccccc23)cc1 ; c1ccc(-c2ccc3sc4ccc(-c5c6ccccc6c(-c6ccc7c(c6)oc6cccc(-c8c9ccccc9c(-c9ccccc9)c9ccccc89)c67)c6ccccc56)cc4c3c2)cc1]

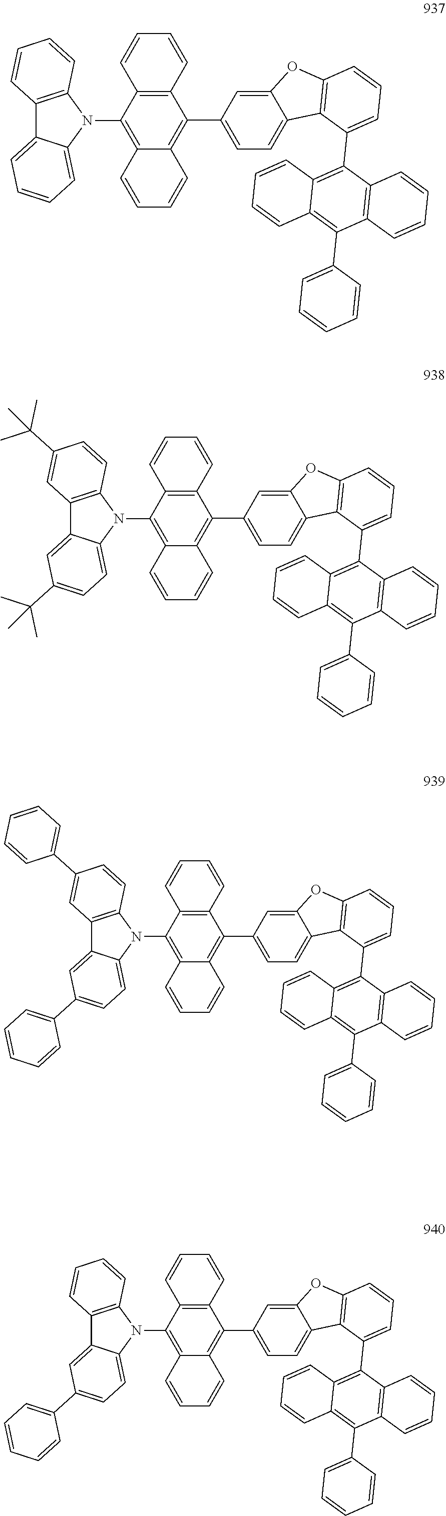 [SMILES: c1ccc(-c2c3ccccc3c(-c3cccc4oc5cc(-c6c7ccccc7c(-n7c8ccccc8c8ccccc87)c7ccccc67)ccc5c34)c3ccccc23)cc1 ; CC(C)(C)c1ccc2c(c1)c1cc(C(C)(C)C)ccc1n2-c1c2ccccc2c(-c2ccc3c(c2)oc2cccc(-c4c5ccccc5c(-c5ccccc5)c5ccccc45)c23)c2ccccc12 ; c1ccc(-c2ccc3c(c2)c2cc(-c4ccccc4)ccc2n3-c2c3ccccc3c(-c3ccc4c(c3)oc3cccc(-c5c6ccccc6c(-c6ccccc6)c6ccccc56)c34)c3ccccc23)cc1 ; c1ccc(-c2ccc3c(c2)c2ccccc2n3-c2c3ccccc3c(-c3ccc4c(c3)oc3cccc(-c5c6ccccc6c(-c6ccccc6)c6ccccc56)c34)c3ccccc23)cc1]

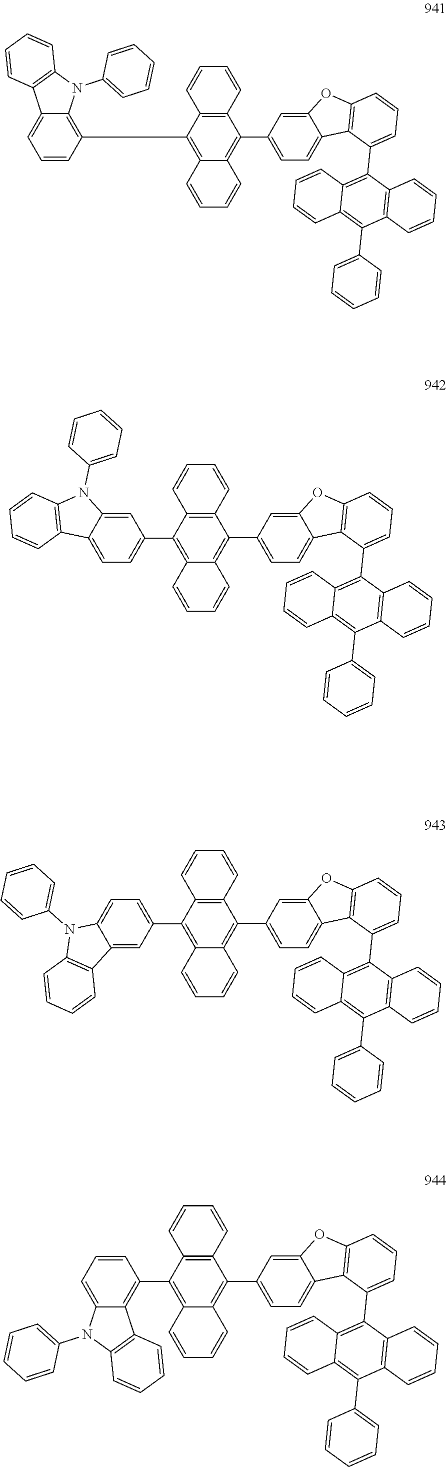 [SMILES: c1ccc(-c2c3ccccc3c(-c3cccc4oc5cc(-c6c7ccccc7c(-c7cccc8c9ccccc9n(-c9ccccc9)c78)c7ccccc67)ccc5c34)c3ccccc23)cc1 ; c1ccc(-c2c3ccccc3c(-c3cccc4oc5cc(-c6c7ccccc7c(-c7ccc8c9ccccc9n(-c9ccccc9)c8c7)c7ccccc67)ccc5c34)c3ccccc23)cc1 ; c1ccc(-c2c3ccccc3c(-c3cccc4oc5cc(-c6c7ccccc7c(-c7ccc8c(c7)c7ccccc7n8-c7ccccc7)c7ccccc67)ccc5c34)c3ccccc23)cc1 ; c1ccc(-c2c3ccccc3c(-c3cccc4oc5cc(-c6c7ccccc7c(-c7cccc8c7c7ccccc7n8-c7ccccc7)c7ccccc67)ccc5c34)c3ccccc23)cc1]

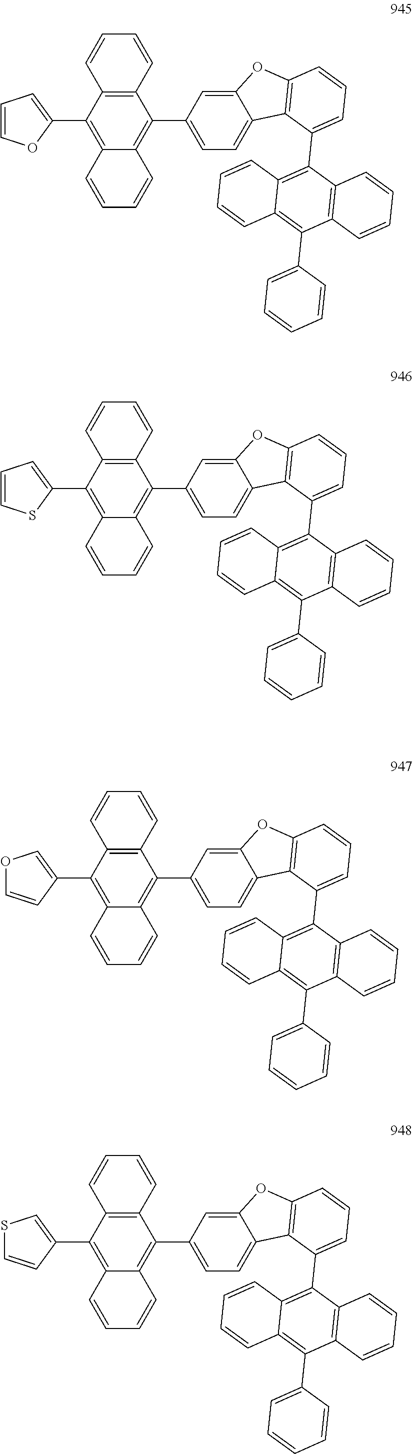 [SMILES: c1ccc(-c2c3ccccc3c(-c3cccc4oc5cc(-c6c7ccccc7c(-c7ccco7)c7ccccc67)ccc5c34)c3ccccc23)cc1 ; c1ccc(-c2c3ccccc3c(-c3cccc4oc5cc(-c6c7ccccc7c(-c7cccs7)c7ccccc67)ccc5c34)c3ccccc23)cc1 ; c1ccc(-c2c3ccccc3c(-c3cccc4oc5cc(-c6c7ccccc7c(-c7ccoc7)c7ccccc67)ccc5c34)c3ccccc23)cc1 ; c1ccc(-c2c3ccccc3c(-c3cccc4oc5cc(-c6c7ccccc7c(-c7ccsc7)c7ccccc67)ccc5c34)c3ccccc23)cc1]

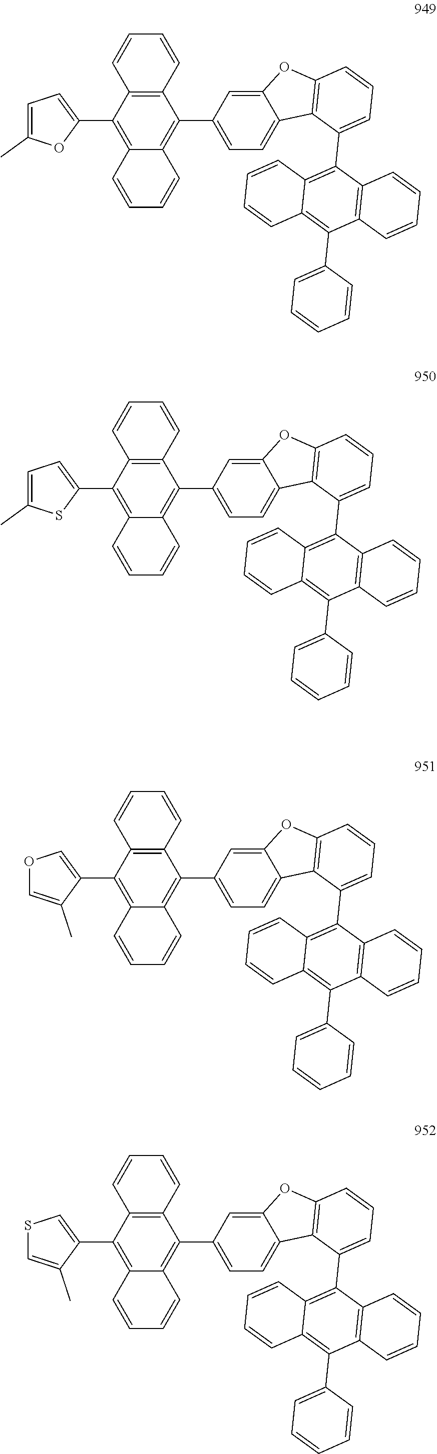 [SMILES: Cc1ccc(-c2c3ccccc3c(-c3ccc4c(c3)oc3cccc(-c5c6ccccc6c(-c6ccccc6)c6ccccc56)c34)c3ccccc23)o1 ; Cc1ccc(-c2c3ccccc3c(-c3ccc4c(c3)oc3cccc(-c5c6ccccc6c(-c6ccccc6)c6ccccc56)c34)c3ccccc23)s1 ; Cc1cocc1-c1c2ccccc2c(-c2ccc3c(c2)oc2cccc(-c4c5ccccc5c(-c5ccccc5)c5ccccc45)c23)c2ccccc12 ; Cc1cscc1-c1c2ccccc2c(-c2ccc3c(c2)oc2cccc(-c4c5ccccc5c(-c5ccccc5)c5ccccc45)c23)c2ccccc12]

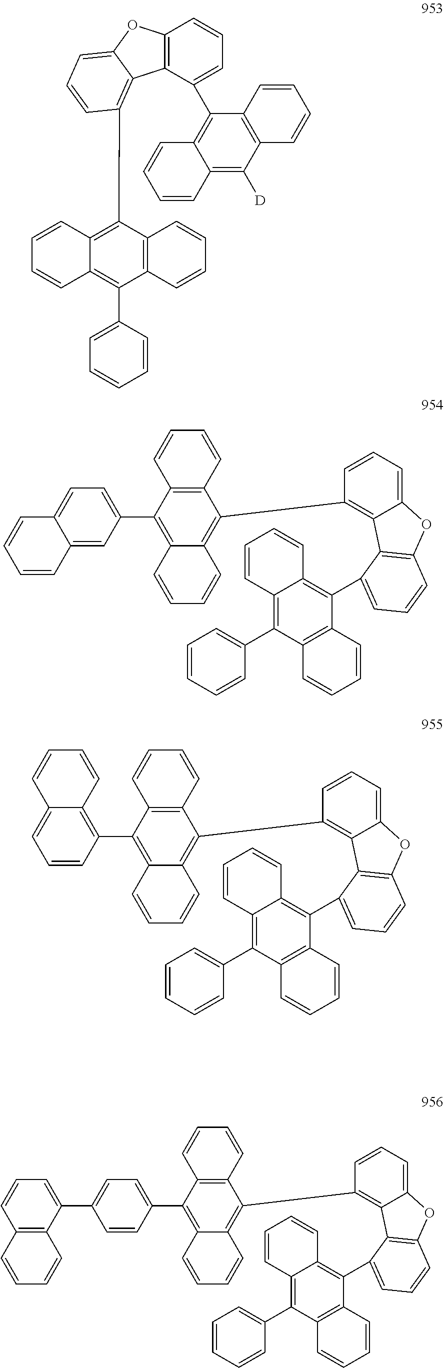 [SMILES: [2H]c1c2ccccc2c(-c2cccc3oc4cccc(-c5c6ccccc6c(-c6ccccc6)c6ccccc56)c4c23)c2ccccc12 ; c1ccc(-c2c3ccccc3c(-c3cccc4oc5cccc(-c6c7ccccc7c(-c7ccc8ccccc8c7)c7ccccc67)c5c34)c3ccccc23)cc1 ; c1ccc(-c2c3ccccc3c(-c3cccc4oc5cccc(-c6c7ccccc7c(-c7cccc8ccccc78)c7ccccc67)c5c34)c3ccccc23)cc1 ; c1ccc(-c2c3ccccc3c(-c3cccc4oc5cccc(-c6c7ccccc7c(-c7ccc(-c8cccc9ccccc89)cc7)c7ccccc67)c5c34)c3ccccc23)cc1]

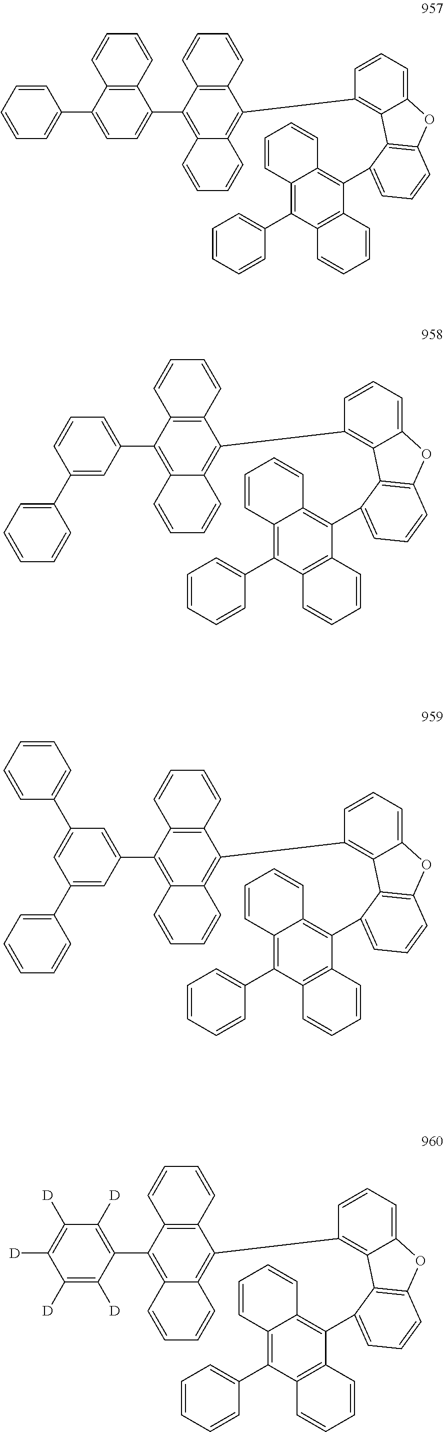 [SMILES: c1ccc(-c2ccc(-c3c4ccccc4c(-c4cccc5oc6cccc(-c7c8ccccc8c(-c8ccccc8)c8ccccc78)c6c45)c4ccccc34)c3ccccc23)cc1 ; c1ccc(-c2cccc(-c3c4ccccc4c(-c4cccc5oc6cccc(-c7c8ccccc8c(-c8ccccc8)c8ccccc78)c6c45)c4ccccc34)c2)cc1 ; c1ccc(-c2cc(-c3ccccc3)cc(-c3c4ccccc4c(-c4cccc5oc6cccc(-c7c8ccccc8c(-c8ccccc8)c8ccccc78)c6c45)c4ccccc34)c2)cc1 ; [2H]c1c([2H])c([2H])c(-c2c3ccccc3c(-c3cccc4oc5cccc(-c6c7ccccc7c(-c7ccccc7)c7ccccc67)c5c34)c3ccccc23)c([2H])c1[2H]]

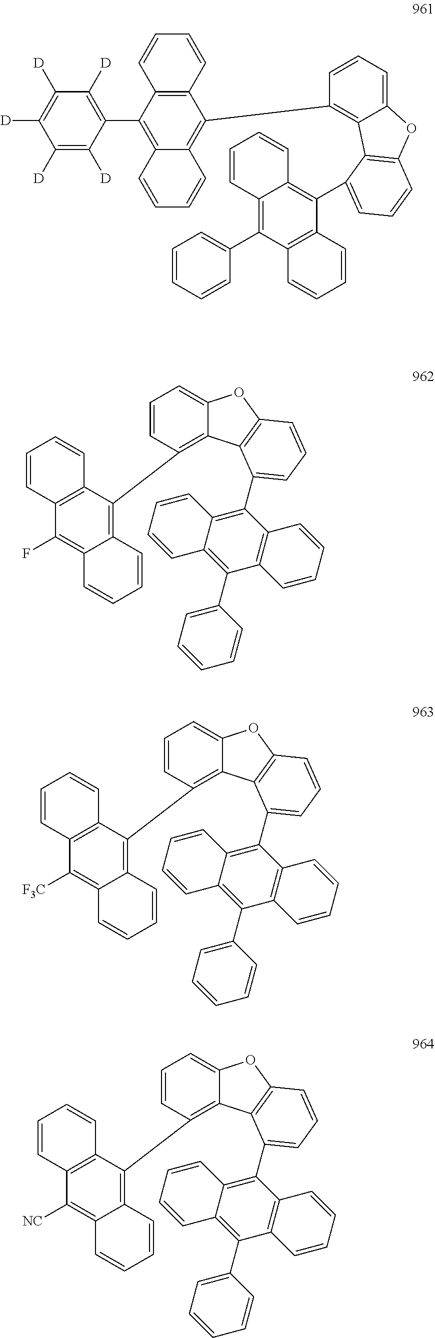 [SMILES: [2H]c1c([2H])c([2H])c(-c2c3ccccc3c(-c3cccc4oc5cccc(-c6c7ccccc7c(-c7ccccc7)c7ccccc67)c5c34)c3ccccc23)c([2H])c1[2H] ; Fc1c2ccccc2c(-c2cccc3oc4cccc(-c5c6ccccc6c(-c6ccccc6)c6ccccc56)c4c23)c2ccccc12 ; FC(F)(F)c1c2ccccc2c(-c2cccc3oc4cccc(-c5c6ccccc6c(-c6ccccc6)c6ccccc56)c4c23)c2ccccc12 ; N#Cc1c2ccccc2c(-c2cccc3oc4cccc(-c5c6ccccc6c(-c6ccccc6)c6ccccc56)c4c23)c2ccccc12]

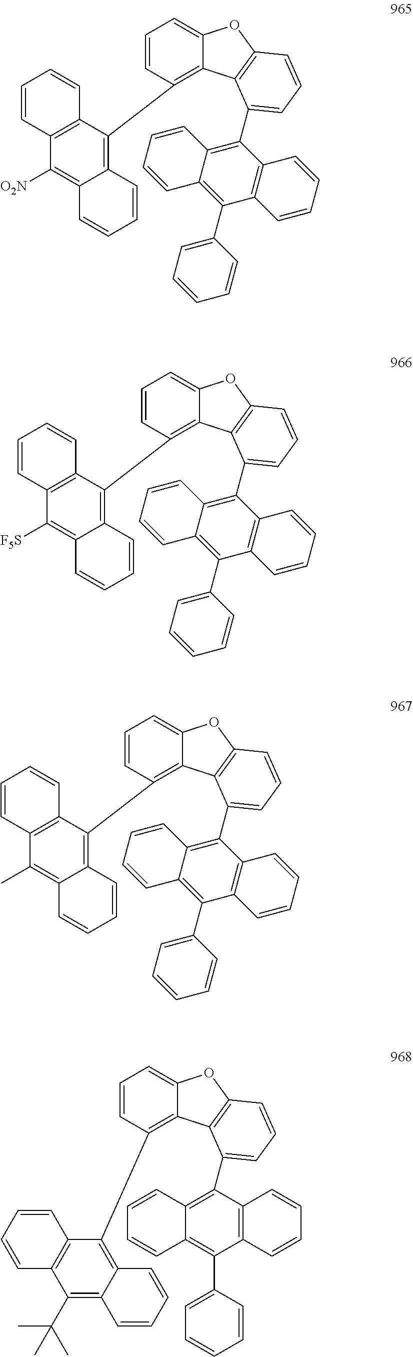

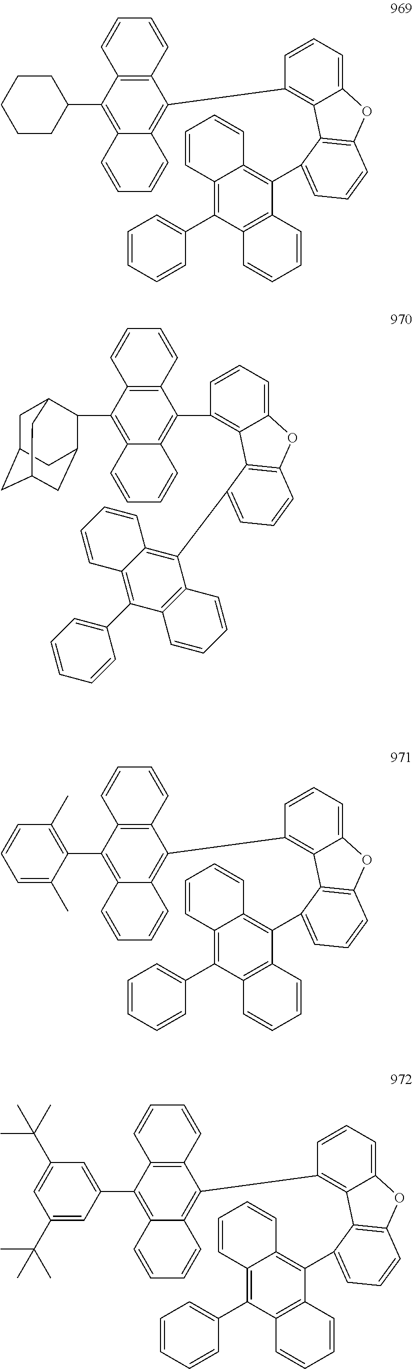 [SMILES: c1ccc(-c2c3ccccc3c(-c3cccc4oc5cccc(-c6c7ccccc7c(C7CCCCC7)c7ccccc67)c5c34)c3ccccc23)cc1 ; c1ccc(-c2c3ccccc3c(-c3cccc4oc5cccc(-c6c7ccccc7c(C7C8CC9CC(C8)CC7C9)c7ccccc67)c5c34)c3ccccc23)cc1 ; Cc1cccc(C)c1-c1c2ccccc2c(-c2cccc3oc4cccc(-c5c6ccccc6c(-c6ccccc6)c6ccccc56)c4c23)c2ccccc12 ; CC(C)(C)c1cc(-c2c3ccccc3c(-c3cccc4oc5cccc(-c6c7ccccc7c(-c7ccccc7)c7ccccc67)c5c34)c3ccccc23)cc(C(C)(C)C)c1]

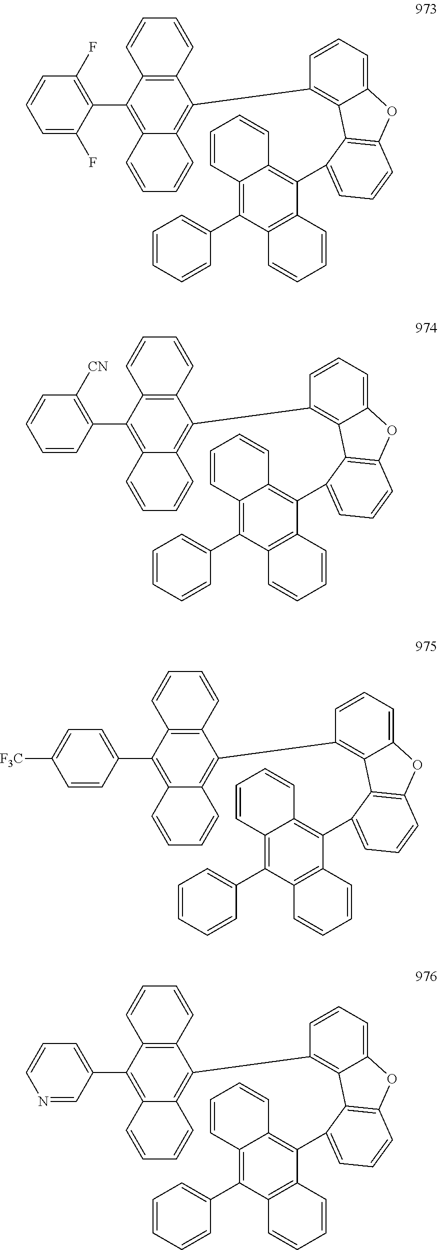 [SMILES: Fc1cccc(F)c1-c1c2ccccc2c(-c2cccc3oc4cccc(-c5c6ccccc6c(-c6ccccc6)c6ccccc56)c4c23)c2ccccc12 ; N#Cc1ccccc1-c1c2ccccc2c(-c2cccc3oc4cccc(-c5c6ccccc6c(-c6ccccc6)c6ccccc56)c4c23)c2ccccc12 ; FC(F)(F)c1ccc(-c2c3ccccc3c(-c3cccc4oc5cccc(-c6c7ccccc7c(-c7ccccc7)c7ccccc67)c5c34)c3ccccc23)cc1 ; c1ccc(-c2c3ccccc3c(-c3cccc4oc5cccc(-c6c7ccccc7c(-c7cccnc7)c7ccccc67)c5c34)c3ccccc23)cc1]

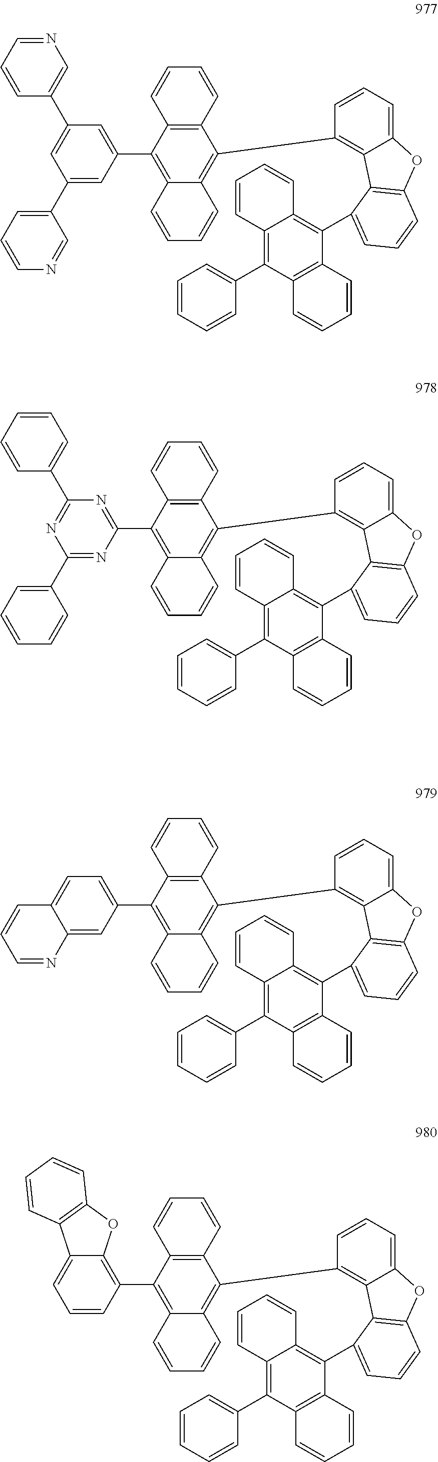 [SMILES: c1ccc(-c2c3ccccc3c(-c3cccc4oc5cccc(-c6c7ccccc7c(-c7cc(-c8cccnc8)cc(-c8cccnc8)c7)c7ccccc67)c5c34)c3ccccc23)cc1 ; c1ccc(-c2nc(-c3ccccc3)nc(-c3c4ccccc4c(-c4cccc5oc6cccc(-c7c8ccccc8c(-c8ccccc8)c8ccccc78)c6c45)c4ccccc34)n2)cc1 ; c1ccc(-c2c3ccccc3c(-c3cccc4oc5cccc(-c6c7ccccc7c(-c7ccc8cccnc8c7)c7ccccc67)c5c34)c3ccccc23)cc1 ; c1ccc(-c2c3ccccc3c(-c3cccc4oc5cccc(-c6c7ccccc7c(-c7cccc8c7oc7ccccc78)c7ccccc67)c5c34)c3ccccc23)cc1]

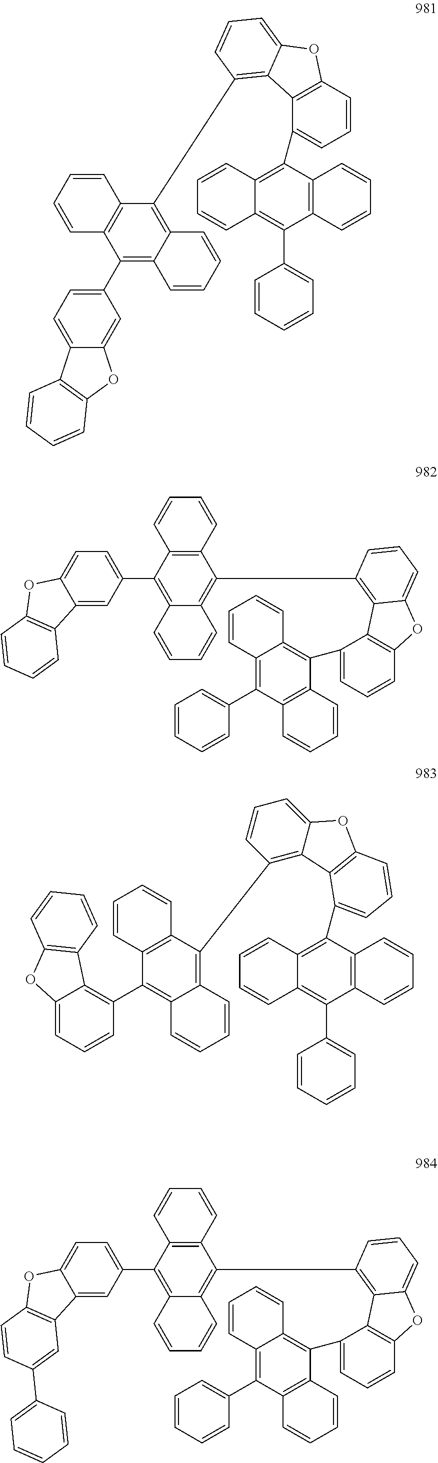 [SMILES: c1ccc(-c2c3ccccc3c(-c3cccc4oc5cccc(-c6c7ccccc7c(-c7ccc8c(c7)oc7ccccc78)c7ccccc67)c5c34)c3ccccc23)cc1 ; c1ccc(-c2c3ccccc3c(-c3cccc4oc5cccc(-c6c7ccccc7c(-c7ccc8oc9ccccc9c8c7)c7ccccc67)c5c34)c3ccccc23)cc1 ; c1ccc(-c2c3ccccc3c(-c3cccc4oc5cccc(-c6c7ccccc7c(-c7cccc8oc9ccccc9c78)c7ccccc67)c5c34)c3ccccc23)cc1 ; c1ccc(-c2ccc3oc4ccc(-c5c6ccccc6c(-c6cccc7oc8cccc(-c9c%10ccccc%10c(-c%10ccccc%10)c%10ccccc9%10)c8c67)c6ccccc56)cc4c3c2)cc1]

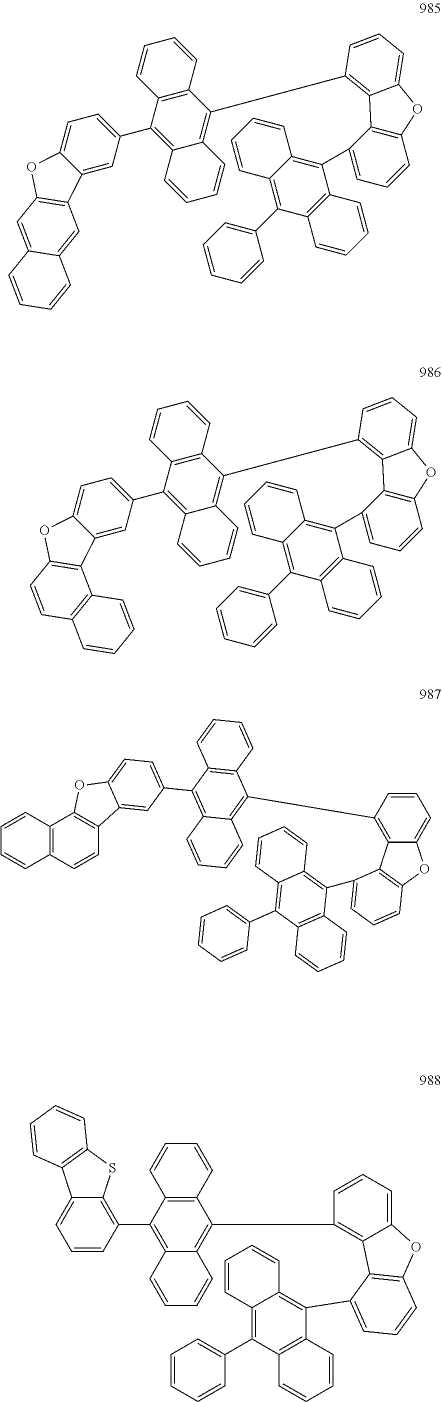 [SMILES: c1ccc(-c2c3ccccc3c(-c3cccc4oc5cccc(-c6c7ccccc7c(-c7ccc8oc9cc%10ccccc%10cc9c8c7)c7ccccc67)c5c34)c3ccccc23)cc1 ; c1ccc(-c2c3ccccc3c(-c3cccc4oc5cccc(-c6c7ccccc7c(-c7ccc8oc9ccc%10ccccc%10c9c8c7)c7ccccc67)c5c34)c3ccccc23)cc1 ; c1ccc(-c2c3ccccc3c(-c3cccc4oc5cccc(-c6c7ccccc7c(-c7ccc8oc9c%10ccccc%10ccc9c8c7)c7ccccc67)c5c34)c3ccccc23)cc1 ; c1ccc(-c2c3ccccc3c(-c3cccc4oc5cccc(-c6c7ccccc7c(-c7cccc8c7sc7ccccc78)c7ccccc67)c5c34)c3ccccc23)cc1]

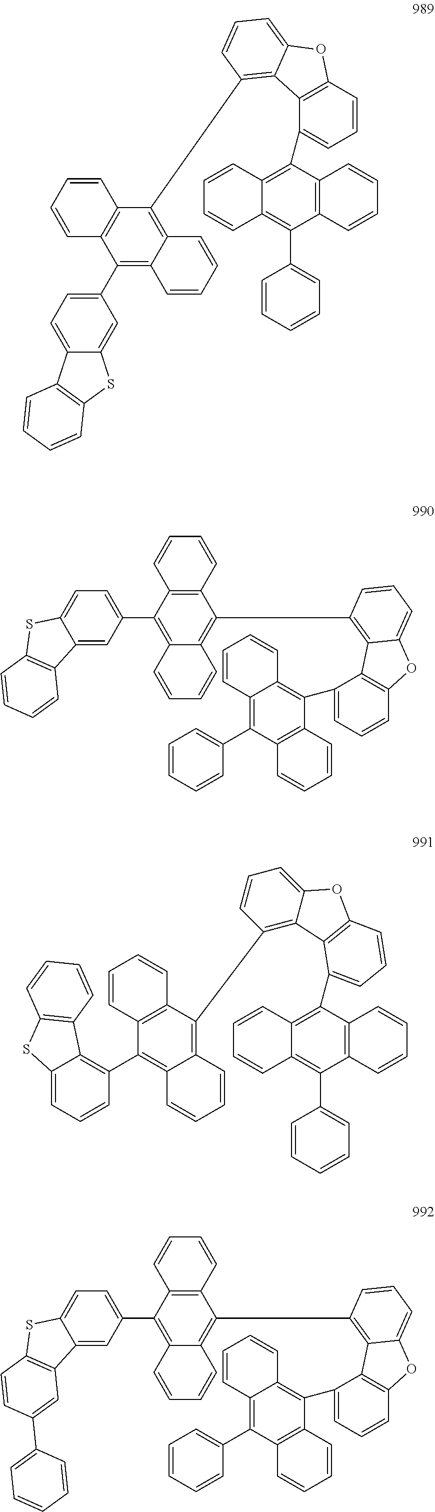 [SMILES: c1ccc(-c2c3ccccc3c(-c3cccc4oc5cccc(-c6c7ccccc7c(-c7ccc8c(c7)sc7ccccc78)c7ccccc67)c5c34)c3ccccc23)cc1 ; c1ccc(-c2c3ccccc3c(-c3cccc4oc5cccc(-c6c7ccccc7c(-c7ccc8sc9ccccc9c8c7)c7ccccc67)c5c34)c3ccccc23)cc1 ; c1ccc(-c2c3ccccc3c(-c3cccc4oc5cccc(-c6c7ccccc7c(-c7cccc8sc9ccccc9c78)c7ccccc67)c5c34)c3ccccc23)cc1 ; c1ccc(-c2ccc3sc4ccc(-c5c6ccccc6c(-c6cccc7oc8cccc(-c9c%10ccccc%10c(-c%10ccccc%10)c%10ccccc9%10)c8c67)c6ccccc56)cc4c3c2)cc1]

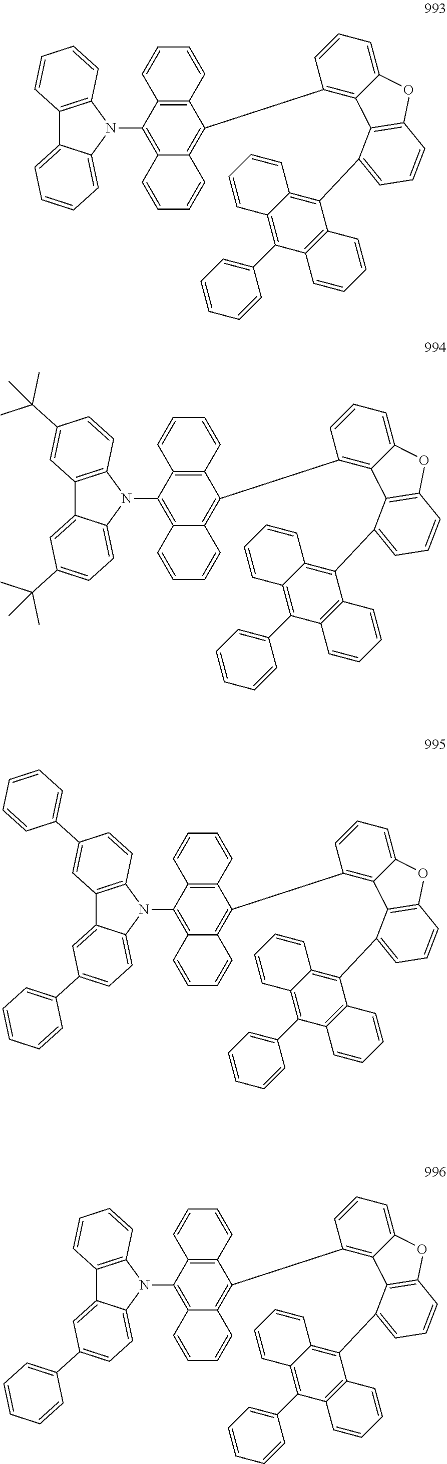 [SMILES: c1ccc(-c2c3ccccc3c(-c3cccc4oc5cccc(-c6c7ccccc7c(-n7c8ccccc8c8ccccc87)c7ccccc67)c5c34)c3ccccc23)cc1 ; CC(C)(C)c1ccc2c(c1)c1cc(C(C)(C)C)ccc1n2-c1c2ccccc2c(-c2cccc3oc4cccc(-c5c6ccccc6c(-c6ccccc6)c6ccccc56)c4c23)c2ccccc12 ; c1ccc(-c2ccc3c(c2)c2cc(-c4ccccc4)ccc2n3-c2c3ccccc3c(-c3cccc4oc5cccc(-c6c7ccccc7c(-c7ccccc7)c7ccccc67)c5c34)c3ccccc23)cc1 ; c1ccc(-c2ccc3c(c2)c2ccccc2n3-c2c3ccccc3c(-c3cccc4oc5cccc(-c6c7ccccc7c(-c7ccccc7)c7ccccc67)c5c34)c3ccccc23)cc1]

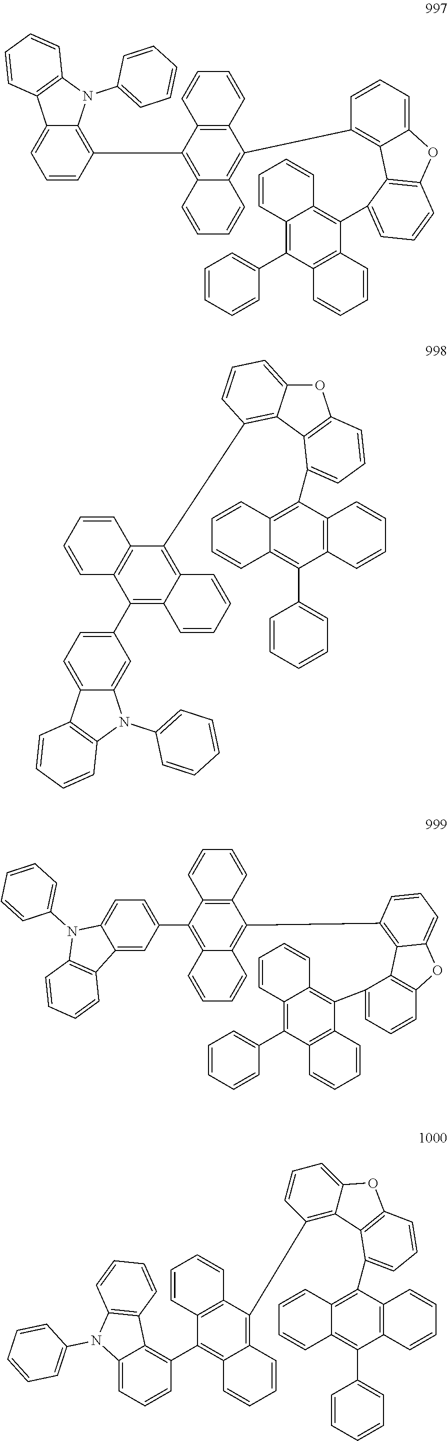 [SMILES: c1ccc(-c2c3ccccc3c(-c3cccc4oc5cccc(-c6c7ccccc7c(-c7cccc8c9ccccc9n(-c9ccccc9)c78)c7ccccc67)c5c34)c3ccccc23)cc1 ; c1ccc(-c2c3ccccc3c(-c3cccc4oc5cccc(-c6c7ccccc7c(-c7ccc8c9ccccc9n(-c9ccccc9)c8c7)c7ccccc67)c5c34)c3ccccc23)cc1 ; c1ccc(-c2c3ccccc3c(-c3cccc4oc5cccc(-c6c7ccccc7c(-c7ccc8c(c7)c7ccccc7n8-c7ccccc7)c7ccccc67)c5c34)c3ccccc23)cc1 ; c1ccc(-c2c3ccccc3c(-c3cccc4oc5cccc(-c6c7ccccc7c(-c7cccc8c7c7ccccc7n8-c7ccccc7)c7ccccc67)c5c34)c3ccccc23)cc1]

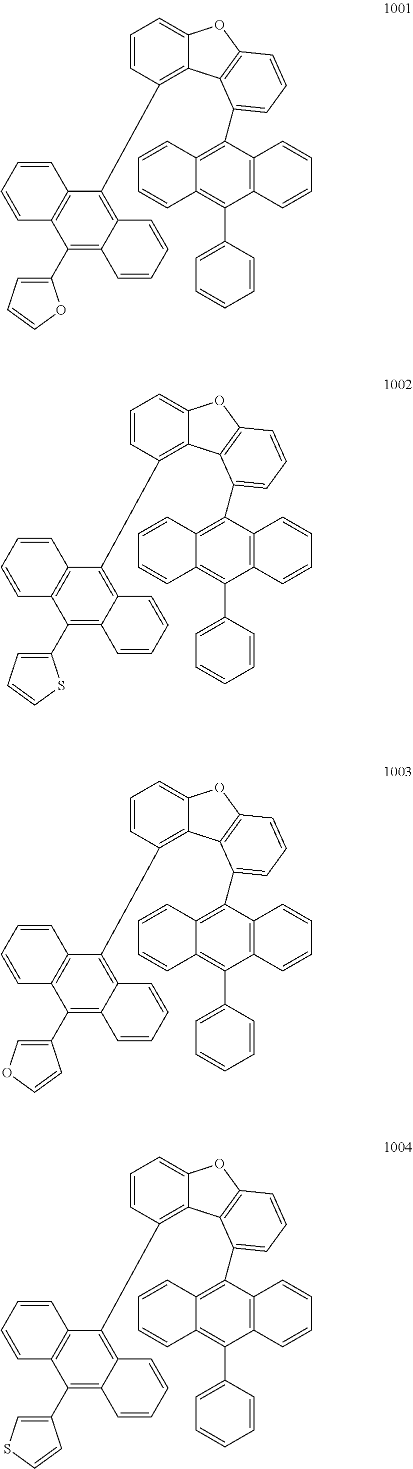 [SMILES: c1ccc(-c2c3ccccc3c(-c3cccc4oc5cccc(-c6c7ccccc7c(-c7ccco7)c7ccccc67)c5c34)c3ccccc23)cc1 ; c1ccc(-c2c3ccccc3c(-c3cccc4oc5cccc(-c6c7ccccc7c(-c7cccs7)c7ccccc67)c5c34)c3ccccc23)cc1 ; c1ccc(-c2c3ccccc3c(-c3cccc4oc5cccc(-c6c7ccccc7c(-c7ccoc7)c7ccccc67)c5c34)c3ccccc23)cc1 ; c1ccc(-c2c3ccccc3c(-c3cccc4oc5cccc(-c6c7ccccc7c(-c7ccsc7)c7ccccc67)c5c34)c3ccccc23)cc1]

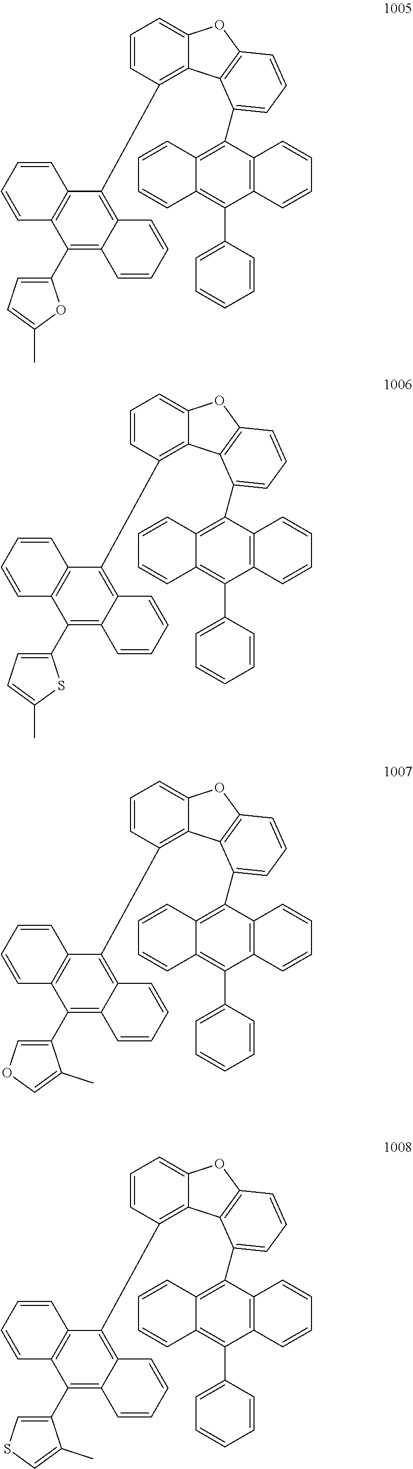 [SMILES: Cc1ccc(-c2c3ccccc3c(-c3cccc4oc5cccc(-c6c7ccccc7c(-c7ccccc7)c7ccccc67)c5c34)c3ccccc23)o1 ; Cc1ccc(-c2c3ccccc3c(-c3cccc4oc5cccc(-c6c7ccccc7c(-c7ccccc7)c7ccccc67)c5c34)c3ccccc23)s1 ; Cc1cocc1-c1c2ccccc2c(-c2cccc3oc4cccc(-c5c6ccccc6c(-c6ccccc6)c6ccccc56)c4c23)c2ccccc12 ; Cc1cscc1-c1c2ccccc2c(-c2cccc3oc4cccc(-c5c6ccccc6c(-c6ccccc6)c6ccccc56)c4c23)c2ccccc12]

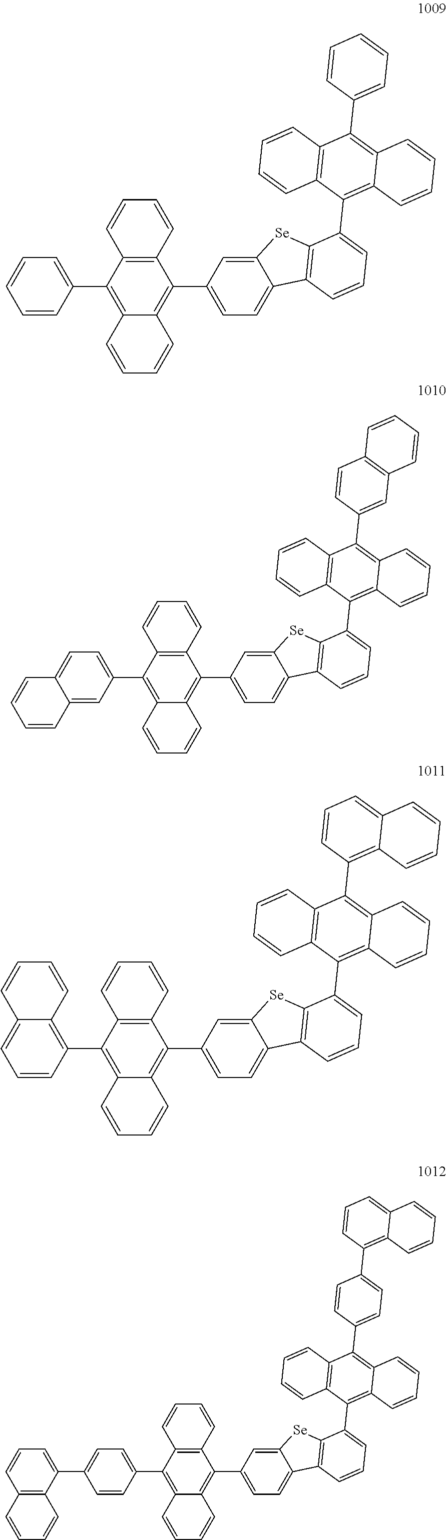 [SMILES: c1ccc(-c2c3ccccc3c(-c3ccc4c(c3)[se]c3c(-c5c6ccccc6c(-c6ccccc6)c6ccccc56)cccc34)c3ccccc23)cc1 ; c1ccc2cc(-c3c4ccccc4c(-c4ccc5c(c4)[se]c4c(-c6c7ccccc7c(-c7ccc8ccccc8c7)c7ccccc67)cccc45)c4ccccc34)ccc2c1 ; c1ccc2c(-c3c4ccccc4c(-c4ccc5c(c4)[se]c4c(-c6c7ccccc7c(-c7cccc8ccccc78)c7ccccc67)cccc45)c4ccccc34)cccc2c1 ; c1ccc2c(-c3ccc(-c4c5ccccc5c(-c5ccc6c(c5)[se]c5c(-c7c8ccccc8c(-c8ccc(-c9cccc%10ccccc9%10)cc8)c8ccccc78)cccc56)c5ccccc45)cc3)cccc2c1]

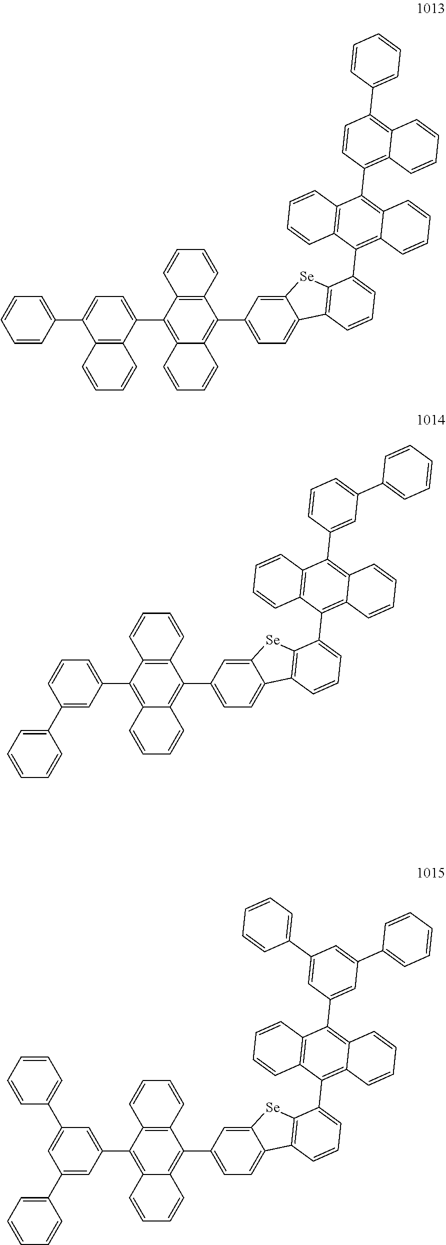 [SMILES: c1ccc(-c2ccc(-c3c4ccccc4c(-c4ccc5c(c4)[se]c4c(-c6c7ccccc7c(-c7ccc(-c8ccccc8)c8ccccc78)c7ccccc67)cccc45)c4ccccc34)c3ccccc23)cc1 ; c1ccc(-c2cccc(-c3c4ccccc4c(-c4ccc5c(c4)[se]c4c(-c6c7ccccc7c(-c7cccc(-c8ccccc8)c7)c7ccccc67)cccc45)c4ccccc34)c2)cc1 ; c1ccc(-c2cc(-c3ccccc3)cc(-c3c4ccccc4c(-c4ccc5c(c4)[se]c4c(-c6c7ccccc7c(-c7cc(-c8ccccc8)cc(-c8ccccc8)c7)c7ccccc67)cccc45)c4ccccc34)c2)cc1]

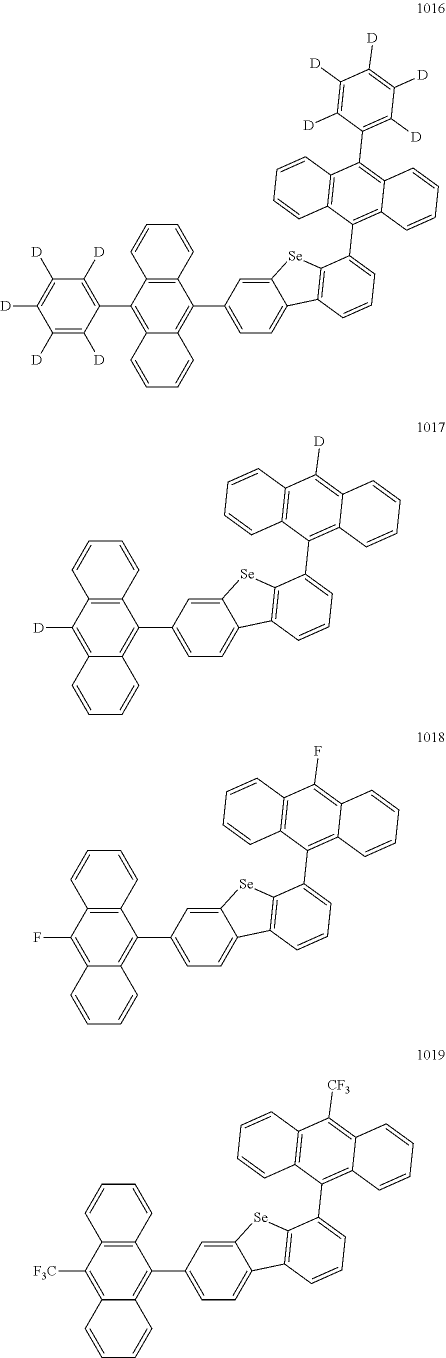 [SMILES: [2H]c1c([2H])c([2H])c(-c2c3ccccc3c(-c3ccc4c(c3)[se]c3c(-c5c6ccccc6c(-c6c([2H])c([2H])c([2H])c([2H])c6[2H])c6ccccc56)cccc34)c3ccccc23)c([2H])c1[2H] ; [2H]c1c2ccccc2c(-c2ccc3c(c2)[se]c2c(-c4c5ccccc5c([2H])c5ccccc45)cccc23)c2ccccc12 ; Fc1c2ccccc2c(-c2ccc3c(c2)[se]c2c(-c4c5ccccc5c(F)c5ccccc45)cccc23)c2ccccc12 ; FC(F)(F)c1c2ccccc2c(-c2ccc3c(c2)[se]c2c(-c4c5ccccc5c(C(F)(F)F)c5ccccc45)cccc23)c2ccccc12]

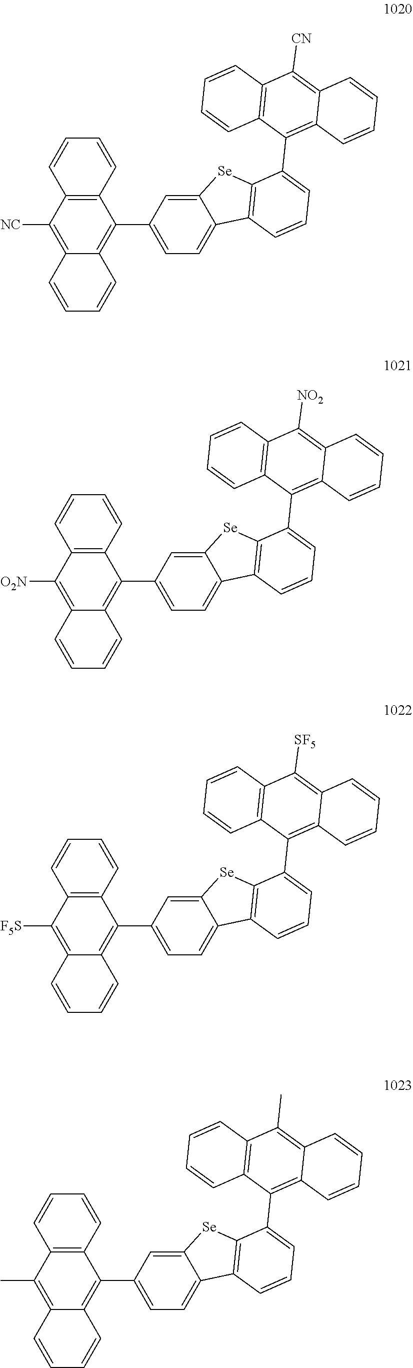

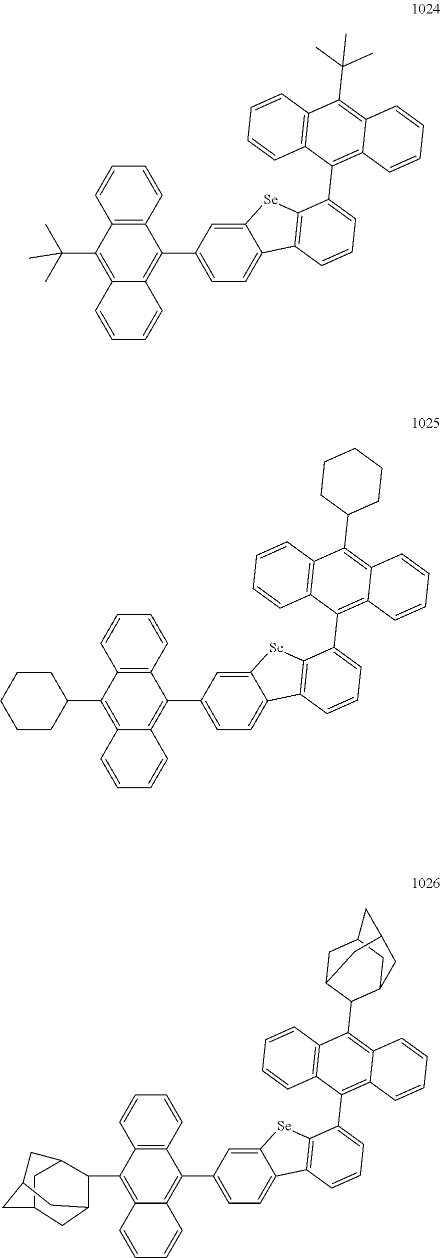 [SMILES: CC(C)(C)c1c2ccccc2c(-c2ccc3c(c2)[se]c2c(-c4c5ccccc5c(C(C)(C)C)c5ccccc45)cccc23)c2ccccc12 ; c1ccc2c(C3CCCCC3)c3ccccc3c(-c3ccc4c(c3)[se]c3c(-c5c6ccccc6c(C6CCCCC6)c6ccccc56)cccc34)c2c1 ; c1ccc2c(C3C4CC5CC(C4)CC3C5)c3ccccc3c(-c3ccc4c(c3)[se]c3c(-c5c6ccccc6c(C6C7CC8CC(C7)CC6C8)c6ccccc56)cccc34)c2c1]

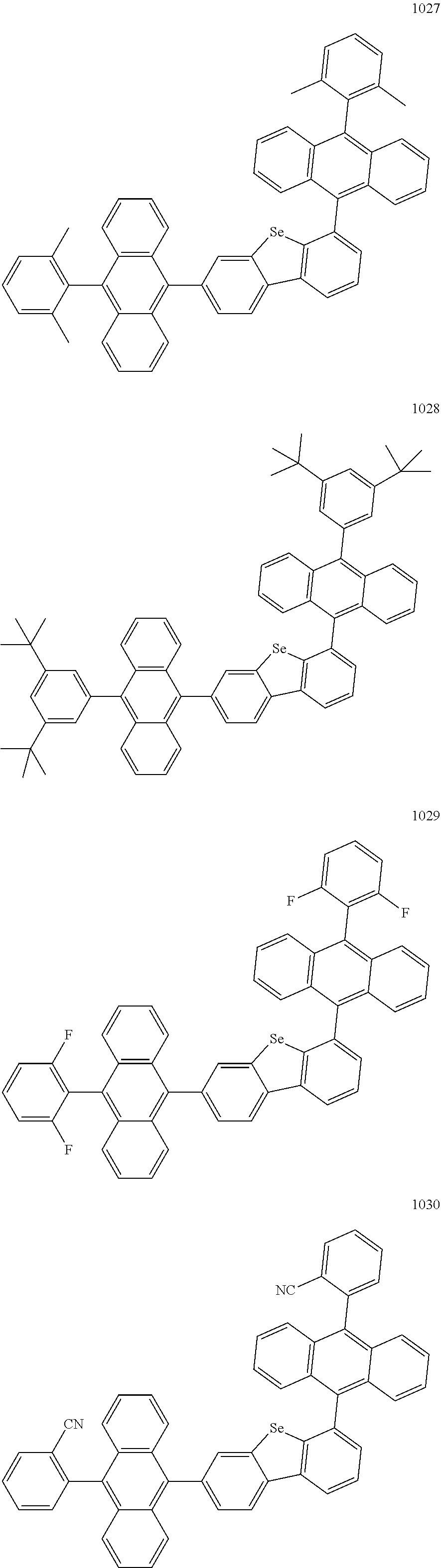 [SMILES: Cc1cccc(C)c1-c1c2ccccc2c(-c2ccc3c(c2)[se]c2c(-c4c5ccccc5c(-c5c(C)cccc5C)c5ccccc45)cccc23)c2ccccc12 ; CC(C)(C)c1cc(-c2c3ccccc3c(-c3ccc4c(c3)[se]c3c(-c5c6ccccc6c(-c6cc(C(C)(C)C)cc(C(C)(C)C)c6)c6ccccc56)cccc34)c3ccccc23)cc(C(C)(C)C)c1 ; Fc1cccc(F)c1-c1c2ccccc2c(-c2ccc3c(c2)[se]c2c(-c4c5ccccc5c(-c5c(F)cccc5F)c5ccccc45)cccc23)c2ccccc12 ; N#Cc1ccccc1-c1c2ccccc2c(-c2ccc3c(c2)[se]c2c(-c4c5ccccc5c(-c5ccccc5C#N)c5ccccc45)cccc23)c2ccccc12]

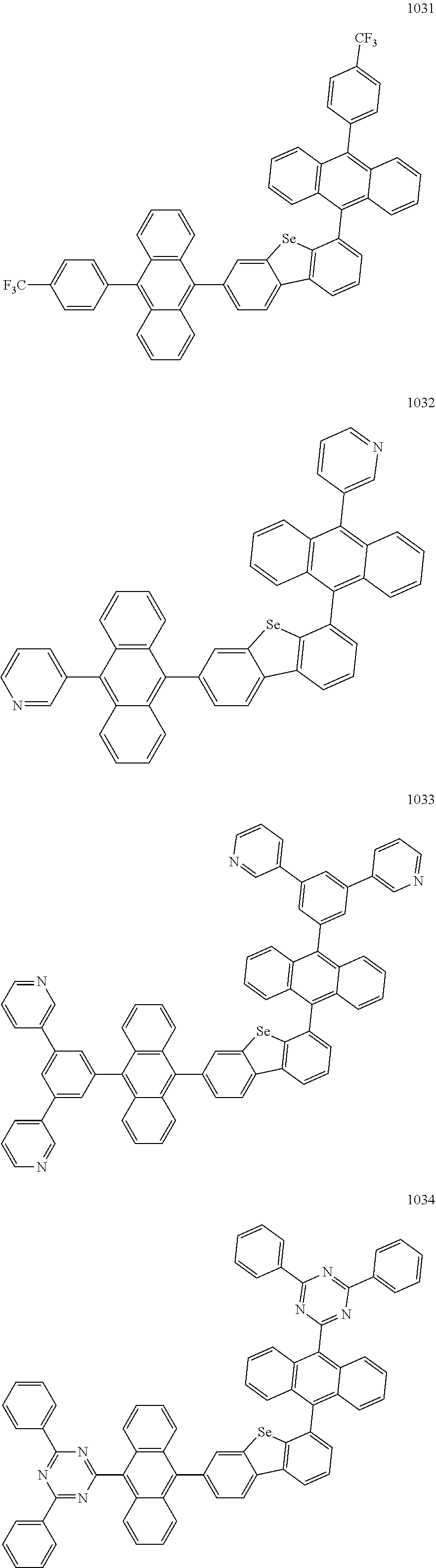 [SMILES: FC(F)(F)c1ccc(-c2c3ccccc3c(-c3ccc4c(c3)[se]c3c(-c5c6ccccc6c(-c6ccc(C(F)(F)F)cc6)c6ccccc56)cccc34)c3ccccc23)cc1 ; c1cncc(-c2c3ccccc3c(-c3ccc4c(c3)[se]c3c(-c5c6ccccc6c(-c6cccnc6)c6ccccc56)cccc34)c3ccccc23)c1 ; c1cncc(-c2cc(-c3cccnc3)cc(-c3c4ccccc4c(-c4ccc5c(c4)[se]c4c(-c6c7ccccc7c(-c7cc(-c8cccnc8)cc(-c8cccnc8)c7)c7ccccc67)cccc45)c4ccccc34)c2)c1 ; c1ccc(-c2nc(-c3ccccc3)nc(-c3c4ccccc4c(-c4ccc5c(c4)[se]c4c(-c6c7ccccc7c(-c7nc(-c8ccccc8)nc(-c8ccccc8)n7)c7ccccc67)cccc45)c4ccccc34)n2)cc1]

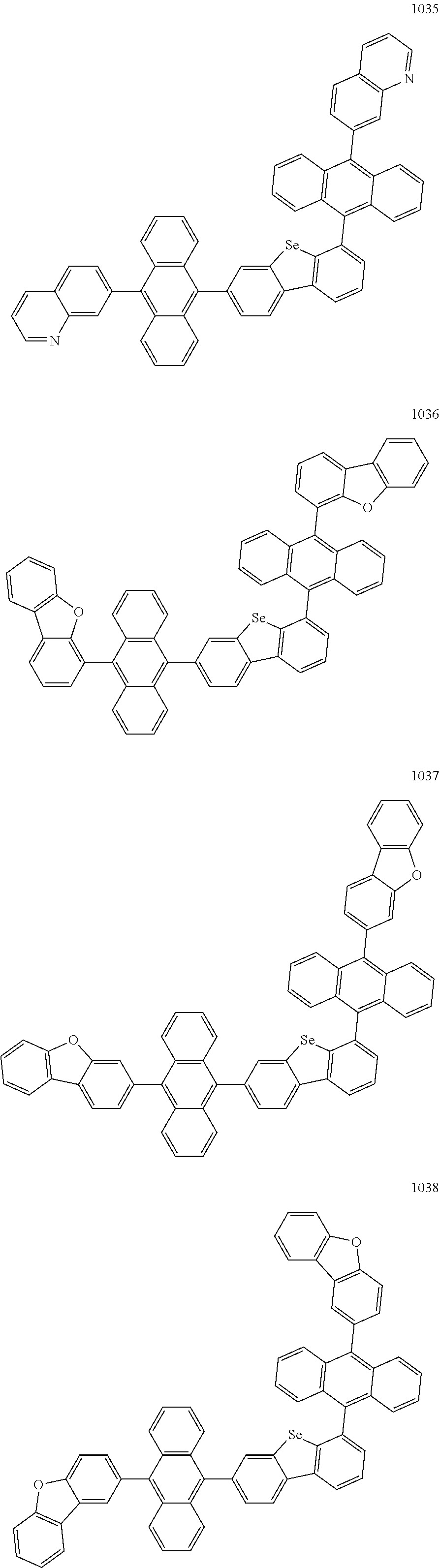 [SMILES: c1cnc2cc(-c3c4ccccc4c(-c4ccc5c(c4)[se]c4c(-c6c7ccccc7c(-c7ccc8cccnc8c7)c7ccccc67)cccc45)c4ccccc34)ccc2c1 ; c1ccc2c(c1)oc1c(-c3c4ccccc4c(-c4ccc5c(c4)[se]c4c(-c6c7ccccc7c(-c7cccc8c7oc7ccccc78)c7ccccc67)cccc45)c4ccccc34)cccc12 ; c1ccc2c(c1)oc1cc(-c3c4ccccc4c(-c4ccc5c(c4)[se]c4c(-c6c7ccccc7c(-c7ccc8c(c7)oc7ccccc78)c7ccccc67)cccc45)c4ccccc34)ccc12 ; c1ccc2c(c1)oc1ccc(-c3c4ccccc4c(-c4ccc5c(c4)[se]c4c(-c6c7ccccc7c(-c7ccc8oc9ccccc9c8c7)c7ccccc67)cccc45)c4ccccc34)cc12]

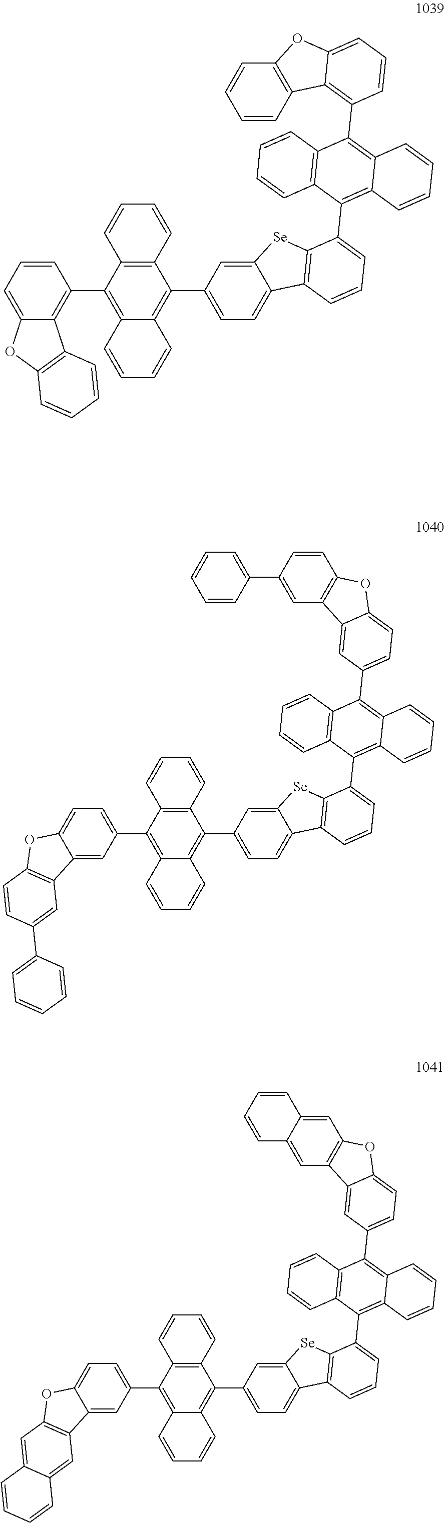 [SMILES: c1ccc2c(c1)oc1cccc(-c3c4ccccc4c(-c4ccc5c(c4)[se]c4c(-c6c7ccccc7c(-c7cccc8oc9ccccc9c78)c7ccccc67)cccc45)c4ccccc34)c12 ; c1ccc(-c2ccc3oc4ccc(-c5c6ccccc6c(-c6ccc7c(c6)[se]c6c(-c8c9ccccc9c(-c9ccc%10oc%11ccc(-c%12ccccc%12)cc%11c%10c9)c9ccccc89)cccc67)c6ccccc56)cc4c3c2)cc1 ; c1ccc2cc3c(cc2c1)oc1ccc(-c2c4ccccc4c(-c4ccc5c(c4)[se]c4c(-c6c7ccccc7c(-c7ccc8oc9cc%10ccccc%10cc9c8c7)c7ccccc67)cccc45)c4ccccc24)cc13]

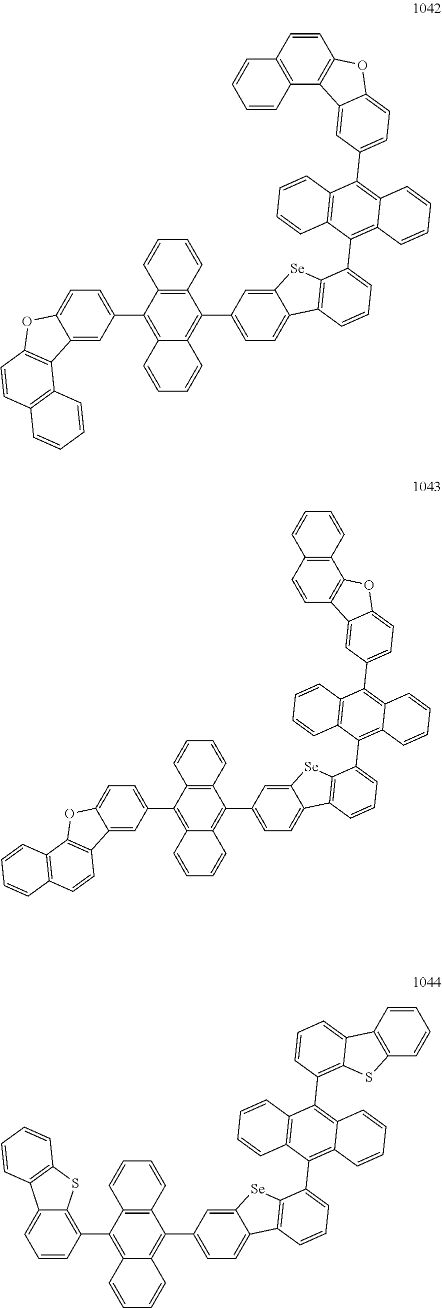 [SMILES: c1ccc2c(c1)ccc1oc3ccc(-c4c5ccccc5c(-c5ccc6c(c5)[se]c5c(-c7c8ccccc8c(-c8ccc9oc%10ccc%11ccccc%11c%10c9c8)c8ccccc78)cccc56)c5ccccc45)cc3c12 ; c1ccc2c(c1)ccc1c3cc(-c4c5ccccc5c(-c5ccc6c(c5)[se]c5c(-c7c8ccccc8c(-c8ccc9oc%10c%11ccccc%11ccc%10c9c8)c8ccccc78)cccc56)c5ccccc45)ccc3oc21 ; c1ccc2c(c1)sc1c(-c3c4ccccc4c(-c4ccc5c(c4)[se]c4c(-c6c7ccccc7c(-c7cccc8c7sc7ccccc78)c7ccccc67)cccc45)c4ccccc34)cccc12]

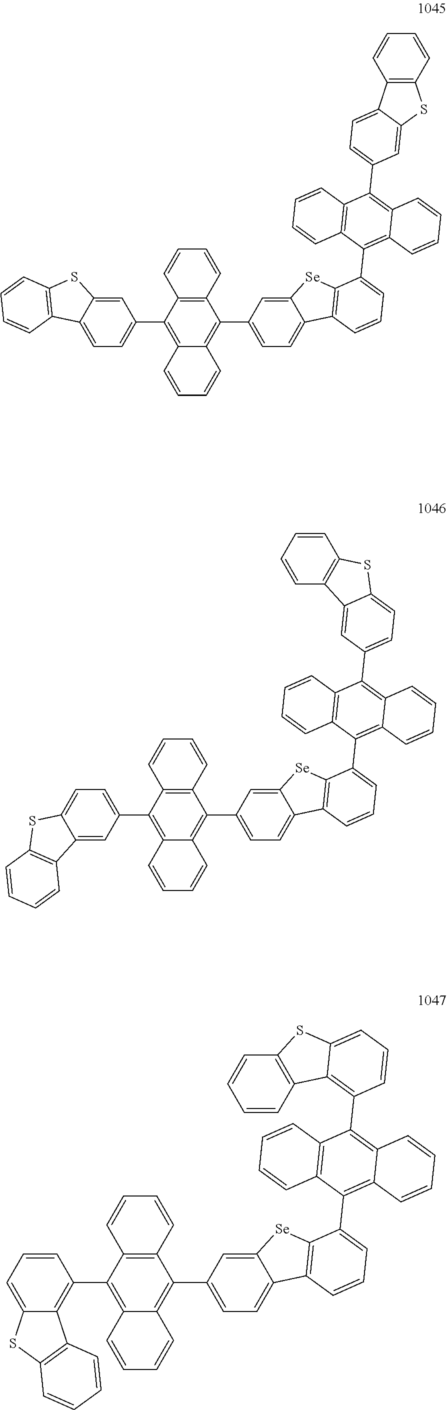 [SMILES: c1ccc2c(c1)sc1cc(-c3c4ccccc4c(-c4ccc5c(c4)[se]c4c(-c6c7ccccc7c(-c7ccc8c(c7)sc7ccccc78)c7ccccc67)cccc45)c4ccccc34)ccc12 ; c1ccc2c(c1)sc1ccc(-c3c4ccccc4c(-c4ccc5c(c4)[se]c4c(-c6c7ccccc7c(-c7ccc8sc9ccccc9c8c7)c7ccccc67)cccc45)c4ccccc34)cc12 ; c1ccc2c(c1)sc1cccc(-c3c4ccccc4c(-c4ccc5c(c4)[se]c4c(-c6c7ccccc7c(-c7cccc8sc9ccccc9c78)c7ccccc67)cccc45)c4ccccc34)c12]

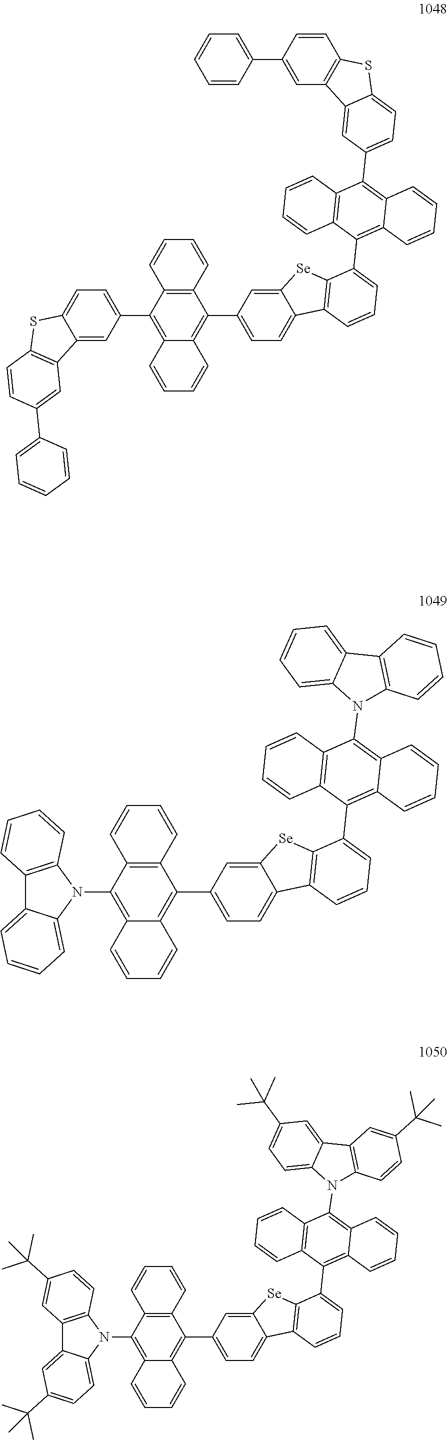 [SMILES: c1ccc(-c2ccc3sc4ccc(-c5c6ccccc6c(-c6ccc7c(c6)[se]c6c(-c8c9ccccc9c(-c9ccc%10sc%11ccc(-c%12ccccc%12)cc%11c%10c9)c9ccccc89)cccc67)c6ccccc56)cc4c3c2)cc1 ; c1ccc2c(-n3c4ccccc4c4ccccc43)c3ccccc3c(-c3ccc4c(c3)[se]c3c(-c5c6ccccc6c(-n6c7ccccc7c7ccccc76)c6ccccc56)cccc34)c2c1 ; CC(C)(C)c1ccc2c(c1)c1cc(C(C)(C)C)ccc1n2-c1c2ccccc2c(-c2ccc3c(c2)[se]c2c(-c4c5ccccc5c(-n5c6ccc(C(C)(C)C)cc6c6cc(C(C)(C)C)ccc65)c5ccccc45)cccc23)c2ccccc12]

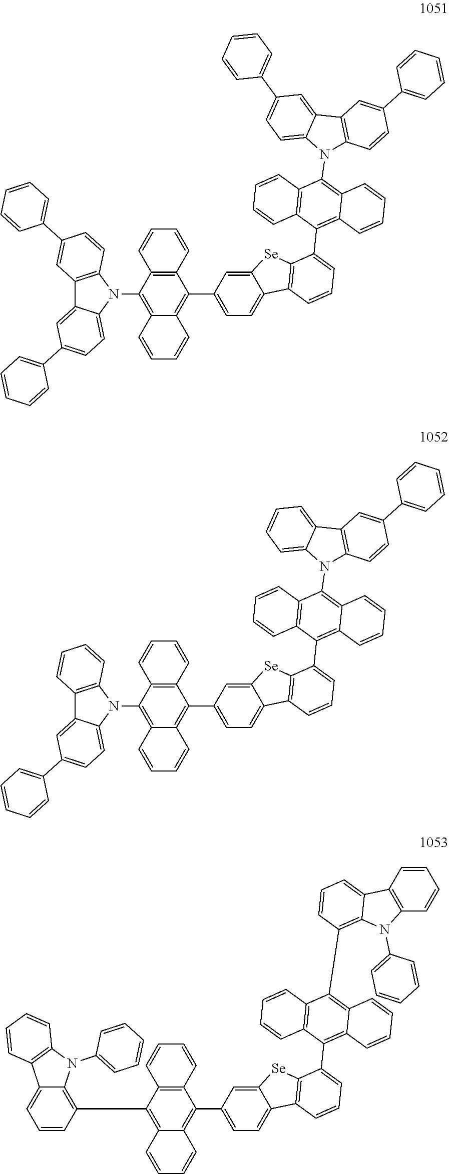 [SMILES: c1ccc(-c2ccc3c(c2)c2cc(-c4ccccc4)ccc2n3-c2c3ccccc3c(-c3ccc4c(c3)[se]c3c(-c5c6ccccc6c(-n6c7ccc(-c8ccccc8)cc7c7cc(-c8ccccc8)ccc76)c6ccccc56)cccc34)c3ccccc23)cc1 ; c1ccc(-c2ccc3c(c2)c2ccccc2n3-c2c3ccccc3c(-c3ccc4c(c3)[se]c3c(-c5c6ccccc6c(-n6c7ccccc7c7cc(-c8ccccc8)ccc76)c6ccccc56)cccc34)c3ccccc23)cc1 ; c1ccc(-n2c3ccccc3c3cccc(-c4c5ccccc5c(-c5ccc6c(c5)[se]c5c(-c7c8ccccc8c(-c8cccc9c%10ccccc%10n(-c%10ccccc%10)c89)c8ccccc78)cccc56)c5ccccc45)c32)cc1]

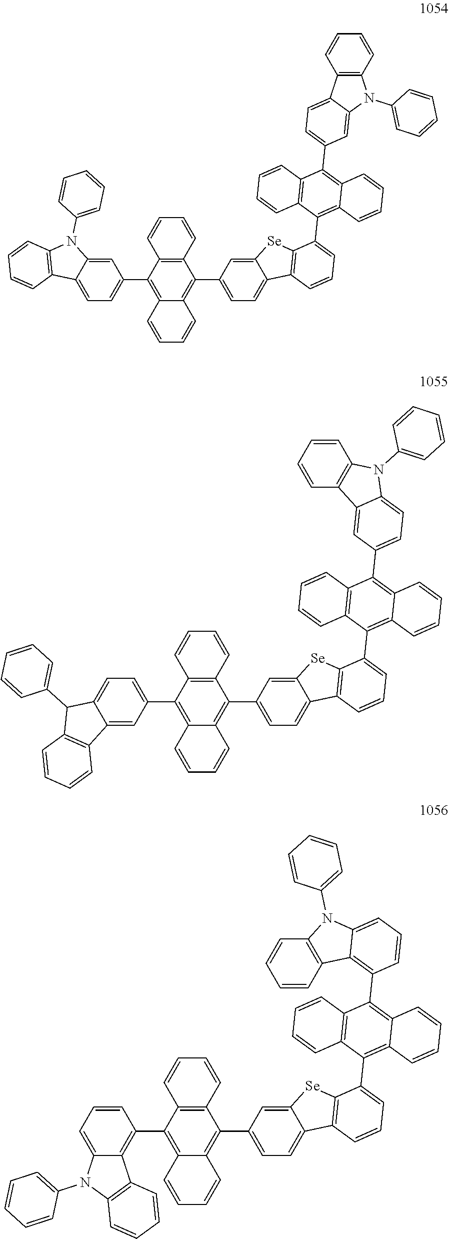 [SMILES: c1ccc(-n2c3ccccc3c3ccc(-c4c5ccccc5c(-c5ccc6c(c5)[se]c5c(-c7c8ccccc8c(-c8ccc9c%10ccccc%10n(-c%10ccccc%10)c9c8)c8ccccc78)cccc56)c5ccccc45)cc32)cc1 ; c1ccc(C2c3ccccc3-c3cc(-c4c5ccccc5c(-c5ccc6c(c5)[se]c5c(-c7c8ccccc8c(-c8ccc9c(c8)c8ccccc8n9-c8ccccc8)c8ccccc78)cccc56)c5ccccc45)ccc32)cc1 ; c1ccc(-n2c3ccccc3c3c(-c4c5ccccc5c(-c5ccc6c(c5)[se]c5c(-c7c8ccccc8c(-c8cccc9c8c8ccccc8n9-c8ccccc8)c8ccccc78)cccc56)c5ccccc45)cccc32)cc1]

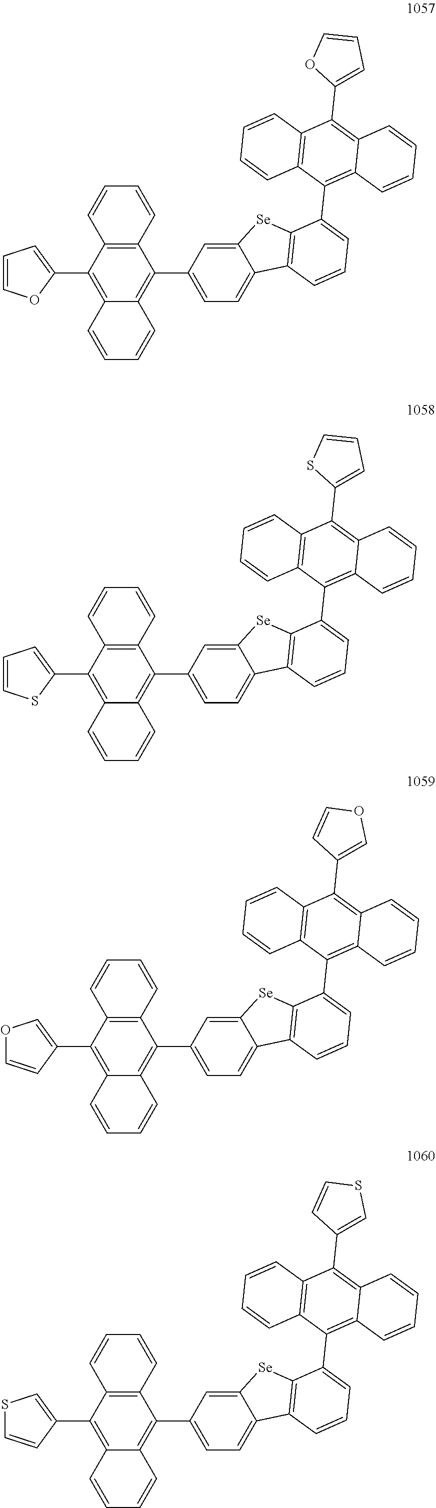 [SMILES: c1coc(-c2c3ccccc3c(-c3ccc4c(c3)[se]c3c(-c5c6ccccc6c(-c6ccco6)c6ccccc56)cccc34)c3ccccc23)c1 ; c1csc(-c2c3ccccc3c(-c3ccc4c(c3)[se]c3c(-c5c6ccccc6c(-c6cccs6)c6ccccc56)cccc34)c3ccccc23)c1 ; c1ccc2c(-c3ccc4c(c3)[se]c3c(-c5c6ccccc6c(-c6ccoc6)c6ccccc56)cccc34)c3ccccc3c(-c3ccoc3)c2c1 ; c1ccc2c(-c3ccc4c(c3)[se]c3c(-c5c6ccccc6c(-c6ccsc6)c6ccccc56)cccc34)c3ccccc3c(-c3ccsc3)c2c1]

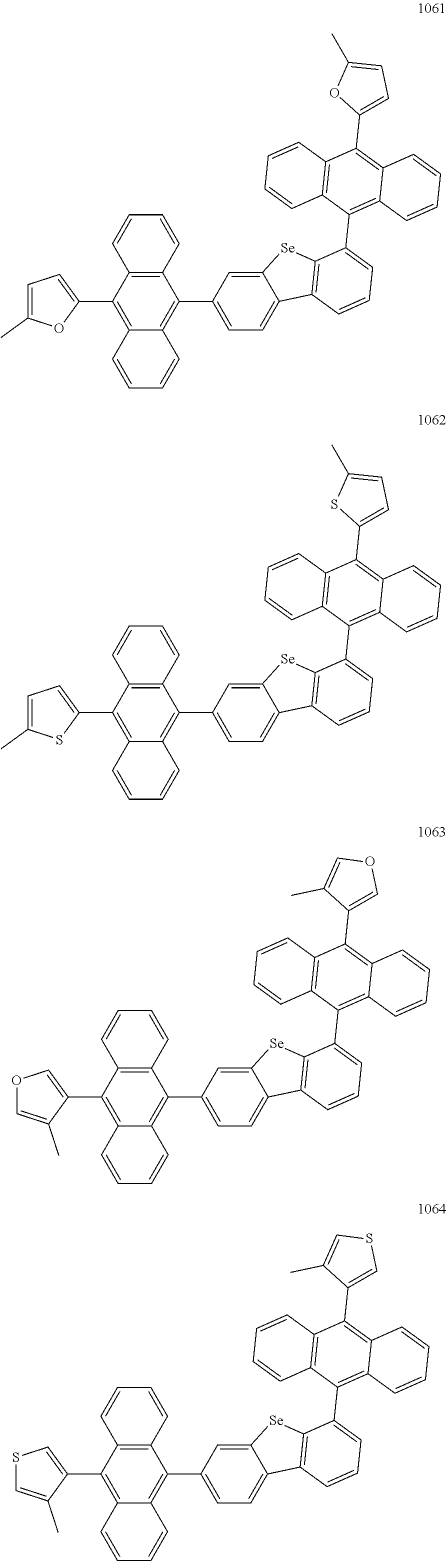 [SMILES: Cc1ccc(-c2c3ccccc3c(-c3ccc4c(c3)[se]c3c(-c5c6ccccc6c(-c6ccc(C)o6)c6ccccc56)cccc34)c3ccccc23)o1 ; Cc1ccc(-c2c3ccccc3c(-c3ccc4c(c3)[se]c3c(-c5c6ccccc6c(-c6ccc(C)s6)c6ccccc56)cccc34)c3ccccc23)s1 ; Cc1cocc1-c1c2ccccc2c(-c2ccc3c(c2)[se]c2c(-c4c5ccccc5c(-c5cocc5C)c5ccccc45)cccc23)c2ccccc12 ; Cc1cscc1-c1c2ccccc2c(-c2ccc3c(c2)[se]c2c(-c4c5ccccc5c(-c5cscc5C)c5ccccc45)cccc23)c2ccccc12]

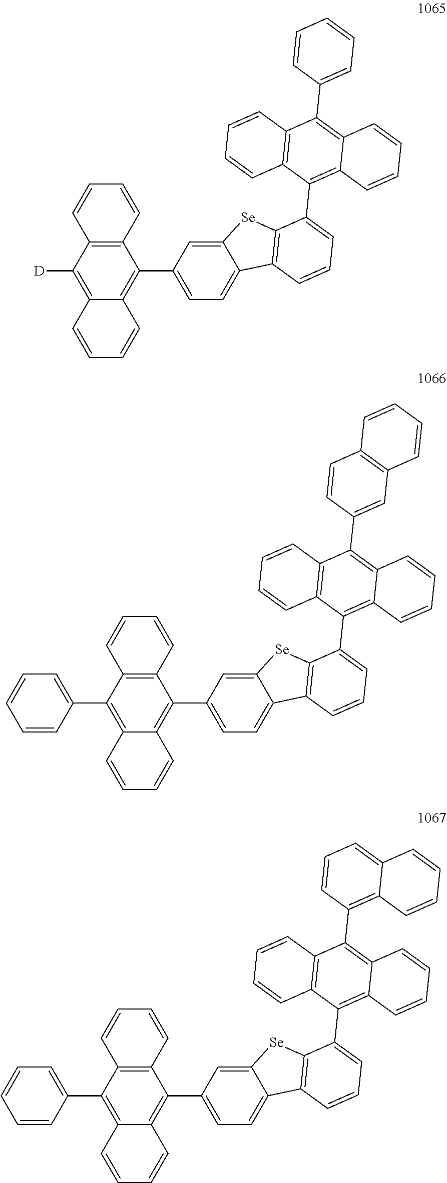 [SMILES: [2H]c1c2ccccc2c(-c2ccc3c(c2)[se]c2c(-c4c5ccccc5c(-c5ccccc5)c5ccccc45)cccc23)c2ccccc12 ; c1ccc(-c2c3ccccc3c(-c3ccc4c(c3)[se]c3c(-c5c6ccccc6c(-c6ccc7ccccc7c6)c6ccccc56)cccc34)c3ccccc23)cc1 ; c1ccc(-c2c3ccccc3c(-c3ccc4c(c3)[se]c3c(-c5c6ccccc6c(-c6cccc7ccccc67)c6ccccc56)cccc34)c3ccccc23)cc1]

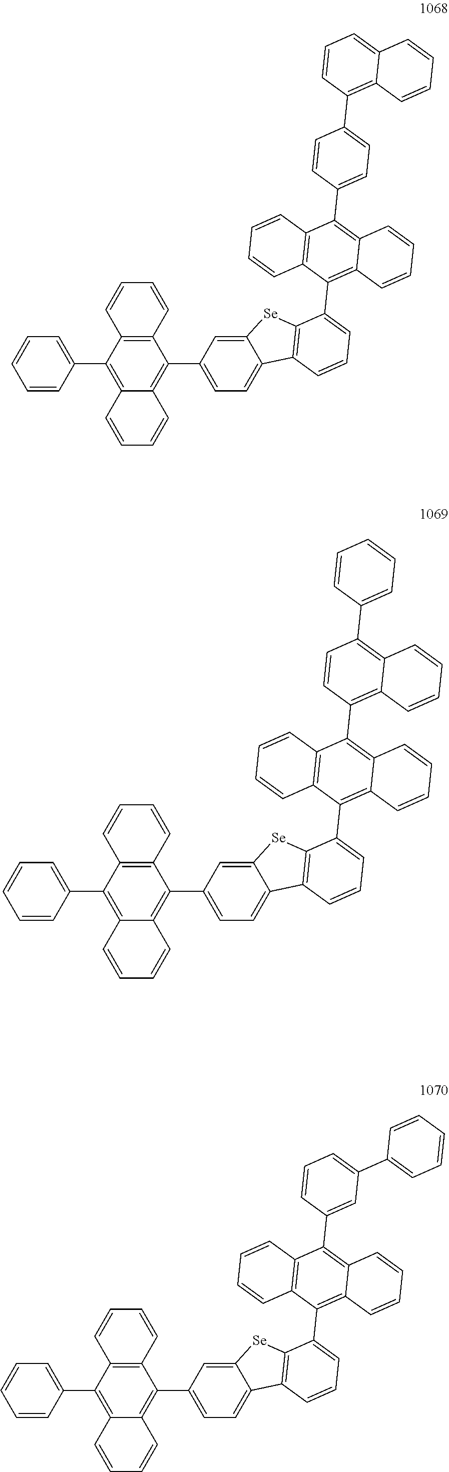 [SMILES: c1ccc(-c2c3ccccc3c(-c3ccc4c(c3)[se]c3c(-c5c6ccccc6c(-c6ccc(-c7cccc8ccccc78)cc6)c6ccccc56)cccc34)c3ccccc23)cc1 ; c1ccc(-c2ccc(-c3c4ccccc4c(-c4cccc5c4[se]c4cc(-c6c7ccccc7c(-c7ccccc7)c7ccccc67)ccc45)c4ccccc34)c3ccccc23)cc1 ; c1ccc(-c2cccc(-c3c4ccccc4c(-c4cccc5c4[se]c4cc(-c6c7ccccc7c(-c7ccccc7)c7ccccc67)ccc45)c4ccccc34)c2)cc1]

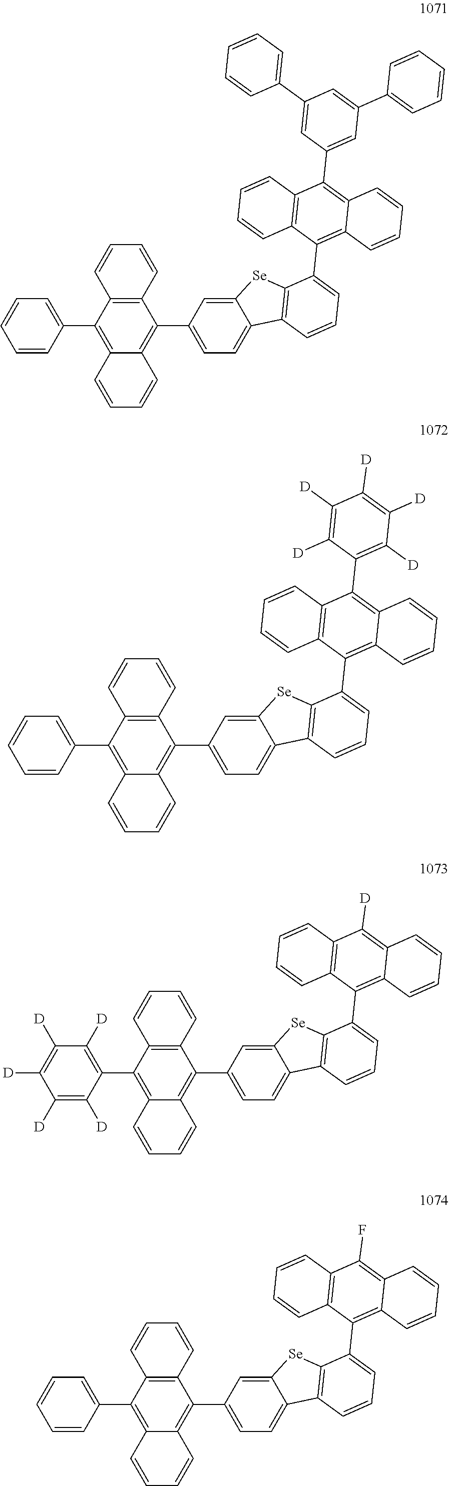 [SMILES: c1ccc(-c2cc(-c3ccccc3)cc(-c3c4ccccc4c(-c4cccc5c4[se]c4cc(-c6c7ccccc7c(-c7ccccc7)c7ccccc67)ccc45)c4ccccc34)c2)cc1 ; [2H]c1c([2H])c([2H])c(-c2c3ccccc3c(-c3cccc4c3[se]c3cc(-c5c6ccccc6c(-c6ccccc6)c6ccccc56)ccc34)c3ccccc23)c([2H])c1[2H] ; [2H]c1c([2H])c([2H])c(-c2c3ccccc3c(-c3ccc4c(c3)[se]c3c(-c5c6ccccc6c([2H])c6ccccc56)cccc34)c3ccccc23)c([2H])c1[2H] ; Fc1c2ccccc2c(-c2cccc3c2[se]c2cc(-c4c5ccccc5c(-c5ccccc5)c5ccccc45)ccc23)c2ccccc12]

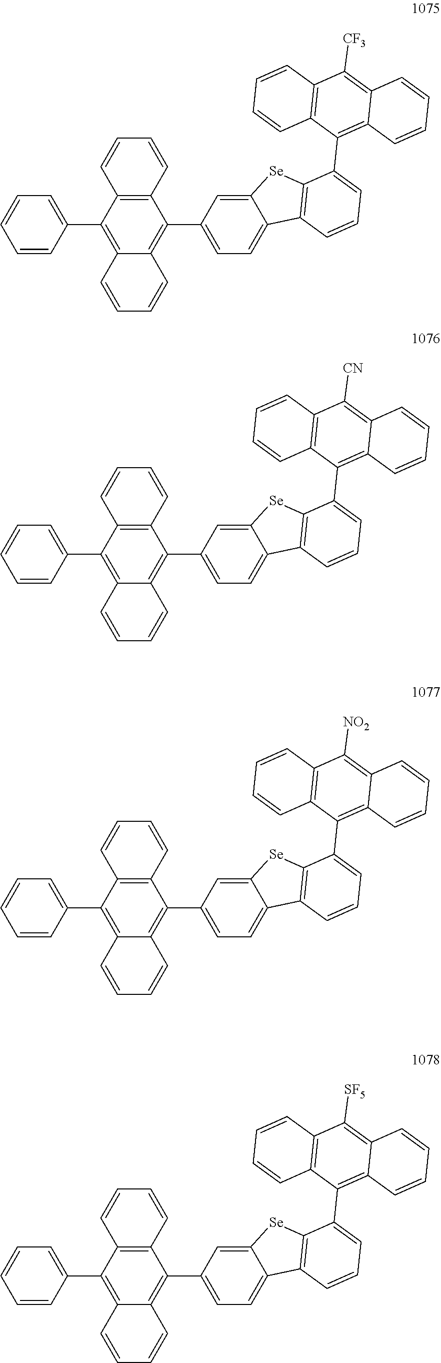

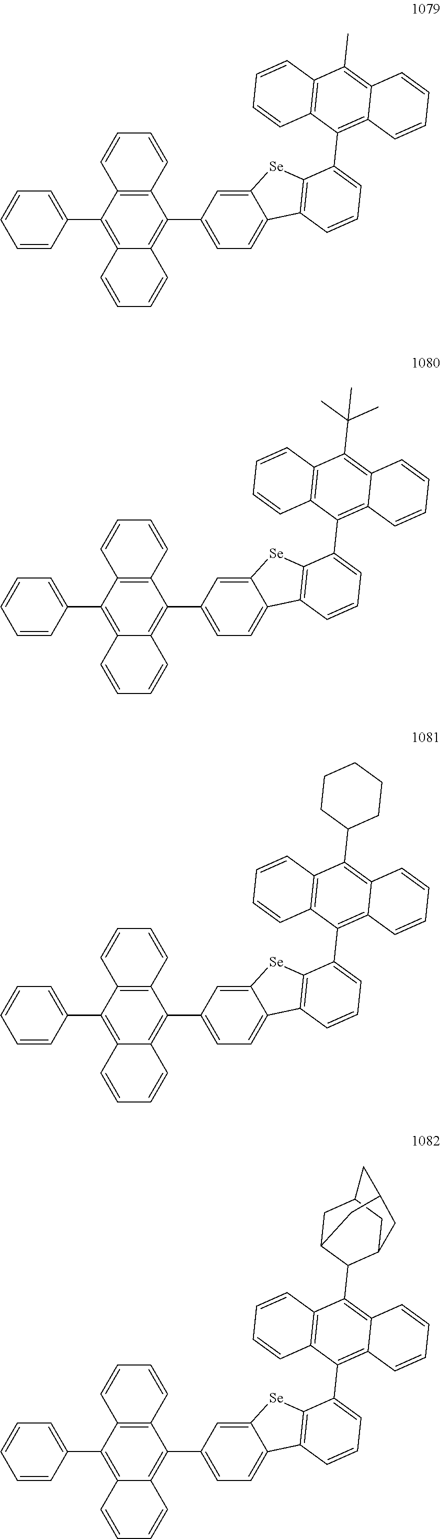 [SMILES: Cc1c2ccccc2c(-c2cccc3c2[se]c2cc(-c4c5ccccc5c(-c5ccccc5)c5ccccc45)ccc23)c2ccccc12 ; CC(C)(C)c1c2ccccc2c(-c2cccc3c2[se]c2cc(-c4c5ccccc5c(-c5ccccc5)c5ccccc45)ccc23)c2ccccc12 ; c1ccc(-c2c3ccccc3c(-c3ccc4c(c3)[se]c3c(-c5c6ccccc6c(C6CCCCC6)c6ccccc56)cccc34)c3ccccc23)cc1 ; c1ccc(-c2c3ccccc3c(-c3ccc4c(c3)[se]c3c(-c5c6ccccc6c(C6C7CC8CC(C7)CC6C8)c6ccccc56)cccc34)c3ccccc23)cc1]

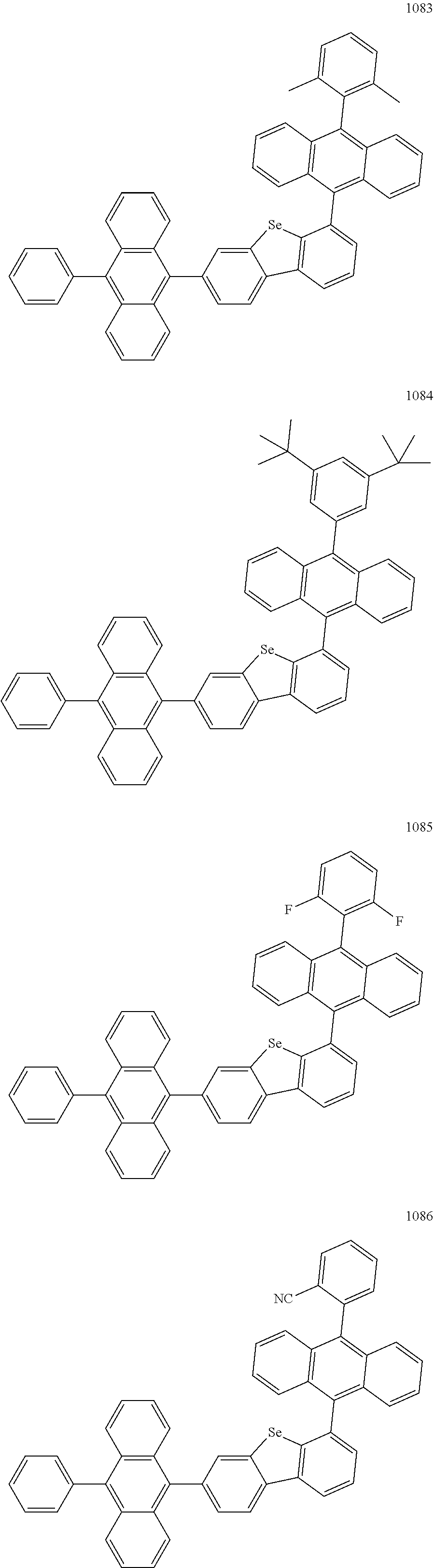 [SMILES: Cc1cccc(C)c1-c1c2ccccc2c(-c2cccc3c2[se]c2cc(-c4c5ccccc5c(-c5ccccc5)c5ccccc45)ccc23)c2ccccc12 ; CC(C)(C)c1cc(-c2c3ccccc3c(-c3cccc4c3[se]c3cc(-c5c6ccccc6c(-c6ccccc6)c6ccccc56)ccc34)c3ccccc23)cc(C(C)(C)C)c1 ; Fc1cccc(F)c1-c1c2ccccc2c(-c2cccc3c2[se]c2cc(-c4c5ccccc5c(-c5ccccc5)c5ccccc45)ccc23)c2ccccc12 ; N#Cc1ccccc1-c1c2ccccc2c(-c2cccc3c2[se]c2cc(-c4c5ccccc5c(-c5ccccc5)c5ccccc45)ccc23)c2ccccc12]

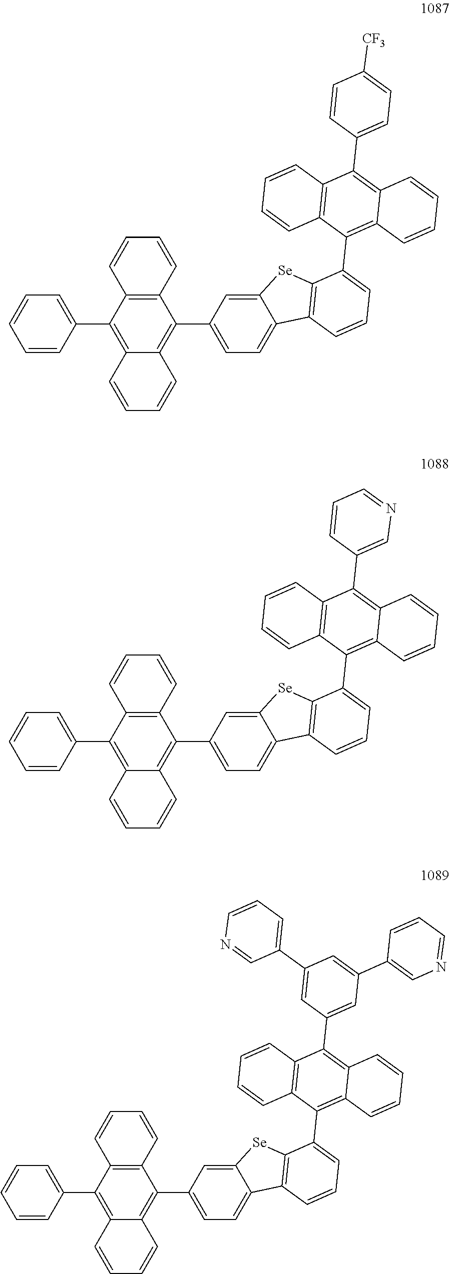 [SMILES: FC(F)(F)c1ccc(-c2c3ccccc3c(-c3cccc4c3[se]c3cc(-c5c6ccccc6c(-c6ccccc6)c6ccccc56)ccc34)c3ccccc23)cc1 ; c1ccc(-c2c3ccccc3c(-c3ccc4c(c3)[se]c3c(-c5c6ccccc6c(-c6cccnc6)c6ccccc56)cccc34)c3ccccc23)cc1 ; c1ccc(-c2c3ccccc3c(-c3ccc4c(c3)[se]c3c(-c5c6ccccc6c(-c6cc(-c7cccnc7)cc(-c7cccnc7)c6)c6ccccc56)cccc34)c3ccccc23)cc1]

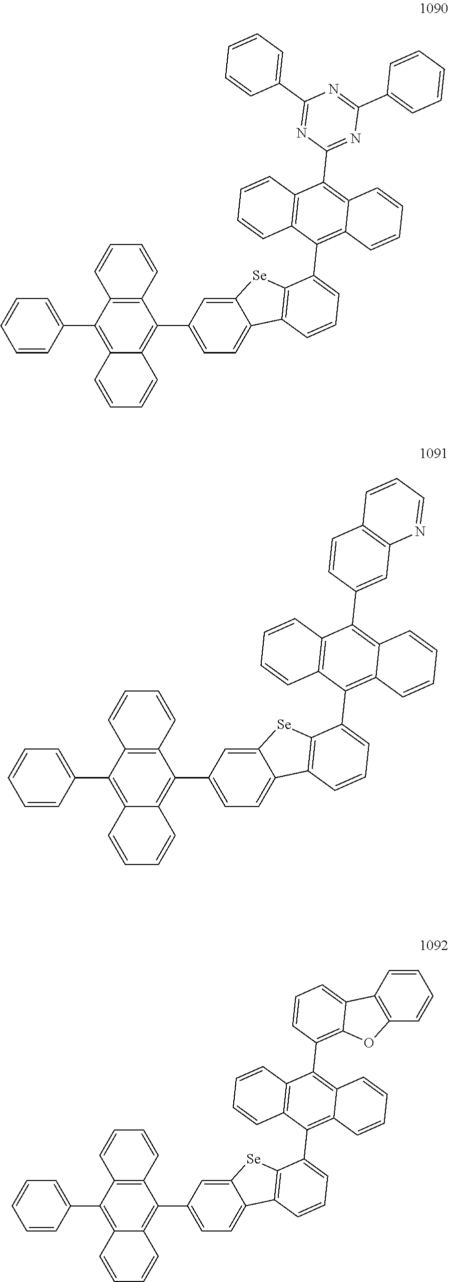 [SMILES: c1ccc(-c2nc(-c3ccccc3)nc(-c3c4ccccc4c(-c4cccc5c4[se]c4cc(-c6c7ccccc7c(-c7ccccc7)c7ccccc67)ccc45)c4ccccc34)n2)cc1 ; c1ccc(-c2c3ccccc3c(-c3ccc4c(c3)[se]c3c(-c5c6ccccc6c(-c6ccc7cccnc7c6)c6ccccc56)cccc34)c3ccccc23)cc1 ; c1ccc(-c2c3ccccc3c(-c3ccc4c(c3)[se]c3c(-c5c6ccccc6c(-c6cccc7c6oc6ccccc67)c6ccccc56)cccc34)c3ccccc23)cc1]

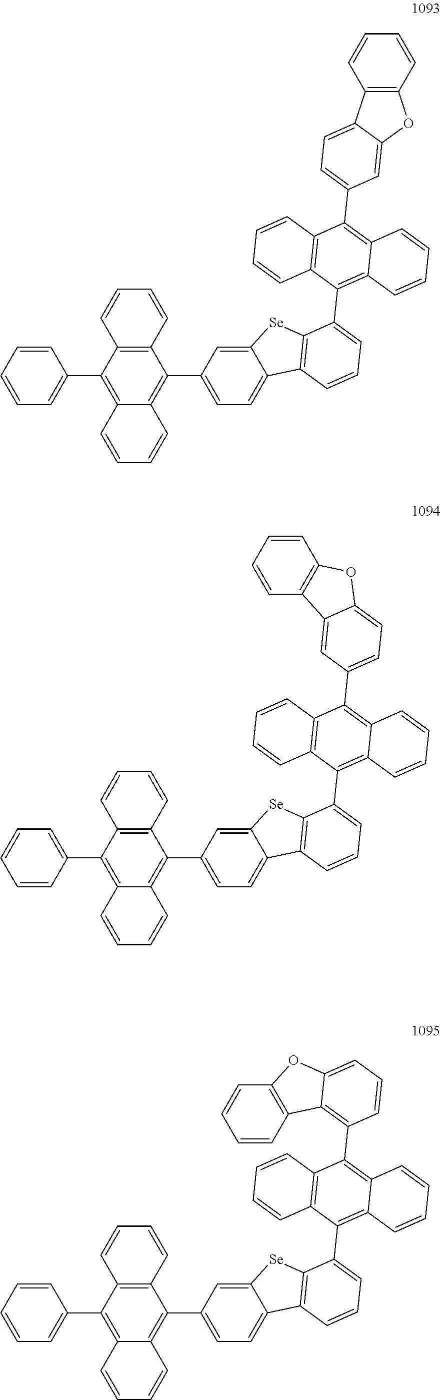 [SMILES: c1ccc(-c2c3ccccc3c(-c3ccc4c(c3)[se]c3c(-c5c6ccccc6c(-c6ccc7c(c6)oc6ccccc67)c6ccccc56)cccc34)c3ccccc23)cc1 ; c1ccc(-c2c3ccccc3c(-c3ccc4c(c3)[se]c3c(-c5c6ccccc6c(-c6ccc7oc8ccccc8c7c6)c6ccccc56)cccc34)c3ccccc23)cc1 ; c1ccc(-c2c3ccccc3c(-c3ccc4c(c3)[se]c3c(-c5c6ccccc6c(-c6cccc7oc8ccccc8c67)c6ccccc56)cccc34)c3ccccc23)cc1]

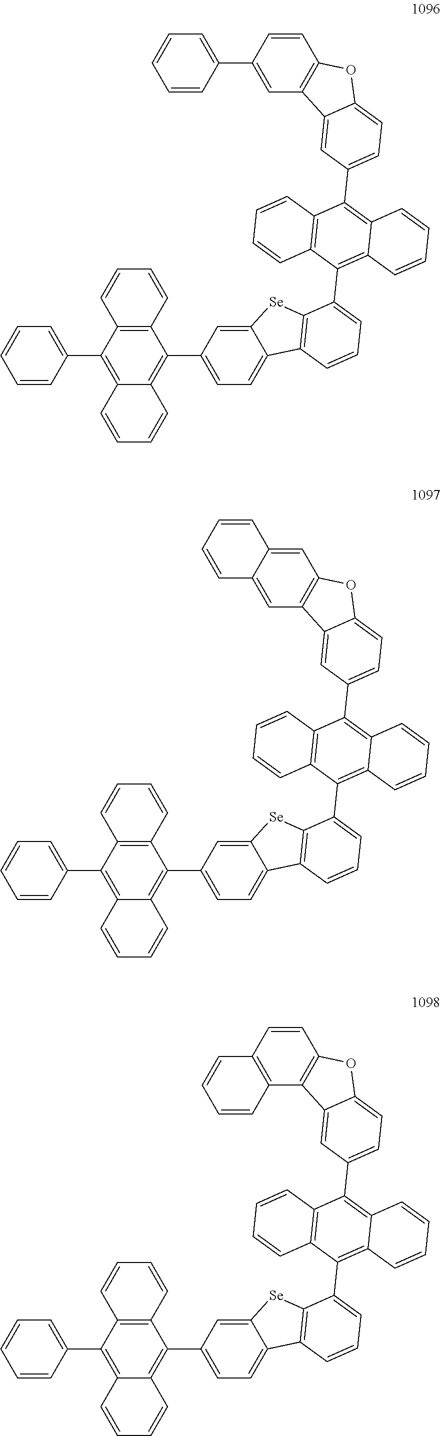 [SMILES: c1ccc(-c2ccc3oc4ccc(-c5c6ccccc6c(-c6cccc7c6[se]c6cc(-c8c9ccccc9c(-c9ccccc9)c9ccccc89)ccc67)c6ccccc56)cc4c3c2)cc1 ; c1ccc(-c2c3ccccc3c(-c3ccc4c(c3)[se]c3c(-c5c6ccccc6c(-c6ccc7oc8cc9ccccc9cc8c7c6)c6ccccc56)cccc34)c3ccccc23)cc1 ; c1ccc(-c2c3ccccc3c(-c3ccc4c(c3)[se]c3c(-c5c6ccccc6c(-c6ccc7oc8ccc9ccccc9c8c7c6)c6ccccc56)cccc34)c3ccccc23)cc1]

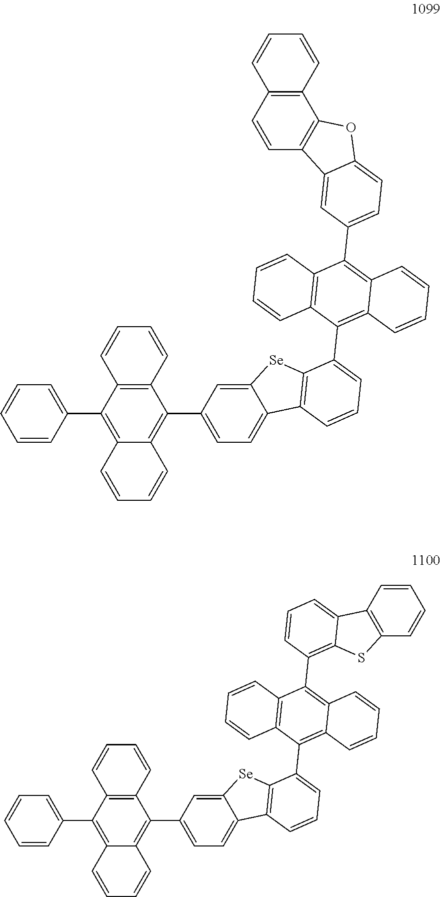 [SMILES: c1ccc(-c2c3ccccc3c(-c3ccc4c(c3)[se]c3c(-c5c6ccccc6c(-c6ccc7oc8c9ccccc9ccc8c7c6)c6ccccc56)cccc34)c3ccccc23)cc1 ; c1ccc(-c2c3ccccc3c(-c3ccc4c(c3)[se]c3c(-c5c6ccccc6c(-c6cccc7c6sc6ccccc67)c6ccccc56)cccc34)c3ccccc23)cc1]

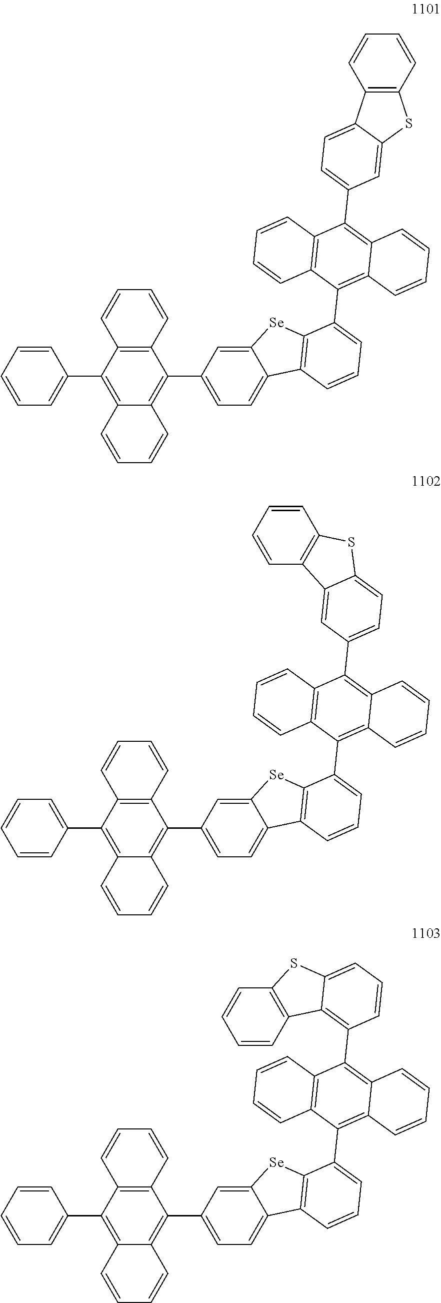 [SMILES: c1ccc(-c2c3ccccc3c(-c3ccc4c(c3)[se]c3c(-c5c6ccccc6c(-c6ccc7c(c6)sc6ccccc67)c6ccccc56)cccc34)c3ccccc23)cc1 ; c1ccc(-c2c3ccccc3c(-c3ccc4c(c3)[se]c3c(-c5c6ccccc6c(-c6ccc7sc8ccccc8c7c6)c6ccccc56)cccc34)c3ccccc23)cc1 ; c1ccc(-c2c3ccccc3c(-c3ccc4c(c3)[se]c3c(-c5c6ccccc6c(-c6cccc7sc8ccccc8c67)c6ccccc56)cccc34)c3ccccc23)cc1]

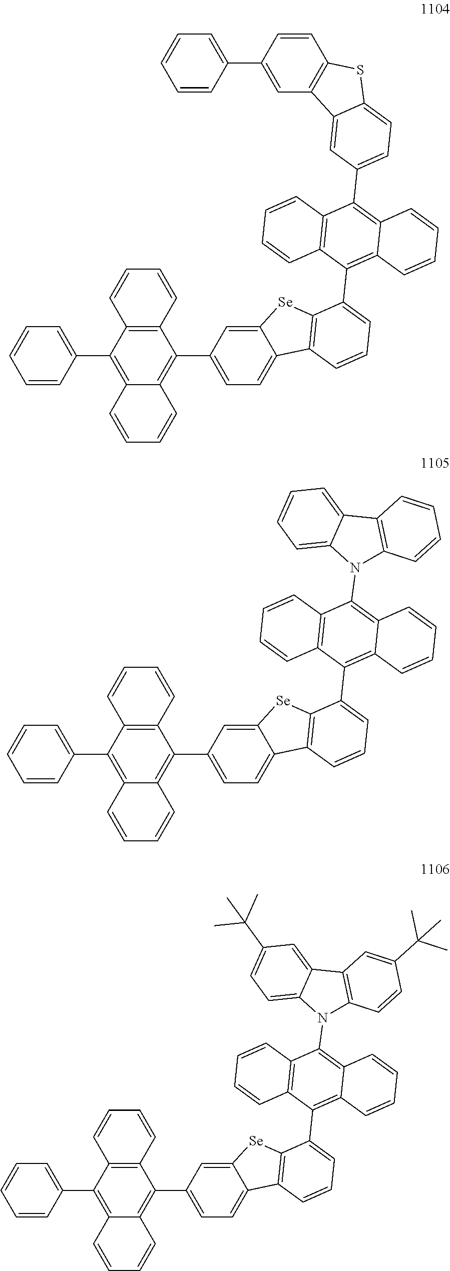 [SMILES: c1ccc(-c2ccc3sc4ccc(-c5c6ccccc6c(-c6cccc7c6[se]c6cc(-c8c9ccccc9c(-c9ccccc9)c9ccccc89)ccc67)c6ccccc56)cc4c3c2)cc1 ; c1ccc(-c2c3ccccc3c(-c3ccc4c(c3)[se]c3c(-c5c6ccccc6c(-n6c7ccccc7c7ccccc76)c6ccccc56)cccc34)c3ccccc23)cc1 ; CC(C)(C)c1ccc2c(c1)c1cc(C(C)(C)C)ccc1n2-c1c2ccccc2c(-c2cccc3c2[se]c2cc(-c4c5ccccc5c(-c5ccccc5)c5ccccc45)ccc23)c2ccccc12]

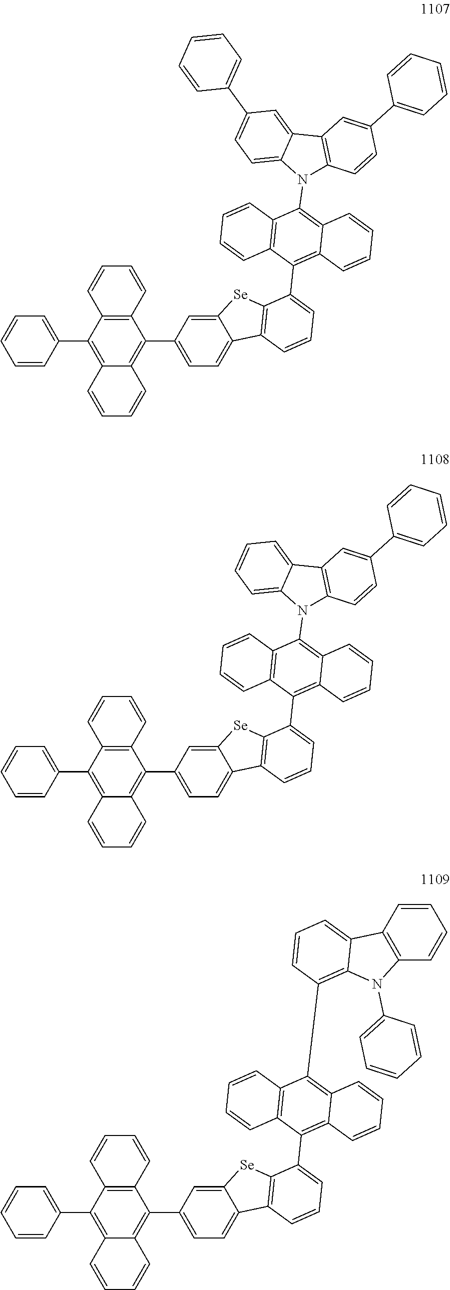 [SMILES: c1ccc(-c2ccc3c(c2)c2cc(-c4ccccc4)ccc2n3-c2c3ccccc3c(-c3cccc4c3[se]c3cc(-c5c6ccccc6c(-c6ccccc6)c6ccccc56)ccc34)c3ccccc23)cc1 ; c1ccc(-c2ccc3c(c2)c2ccccc2n3-c2c3ccccc3c(-c3cccc4c3[se]c3cc(-c5c6ccccc6c(-c6ccccc6)c6ccccc56)ccc34)c3ccccc23)cc1 ; c1ccc(-c2c3ccccc3c(-c3ccc4c(c3)[se]c3c(-c5c6ccccc6c(-c6cccc7c8ccccc8n(-c8ccccc8)c67)c6ccccc56)cccc34)c3ccccc23)cc1]

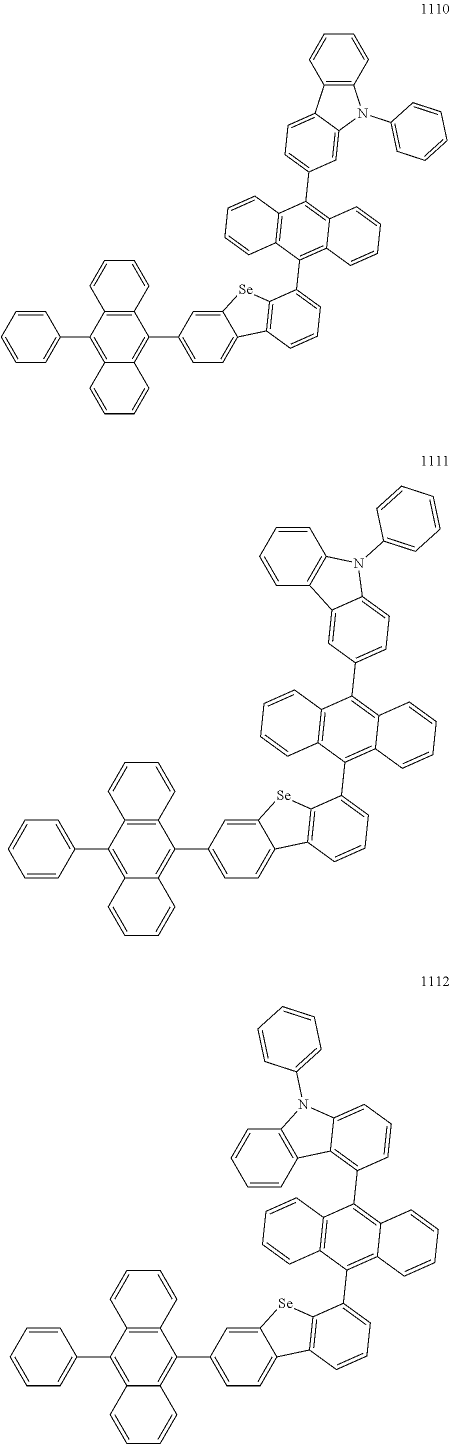 [SMILES: c1ccc(-c2c3ccccc3c(-c3ccc4c(c3)[se]c3c(-c5c6ccccc6c(-c6ccc7c8ccccc8n(-c8ccccc8)c7c6)c6ccccc56)cccc34)c3ccccc23)cc1 ; c1ccc(-c2c3ccccc3c(-c3ccc4c(c3)[se]c3c(-c5c6ccccc6c(-c6ccc7c(c6)c6ccccc6n7-c6ccccc6)c6ccccc56)cccc34)c3ccccc23)cc1 ; c1ccc(-c2c3ccccc3c(-c3ccc4c(c3)[se]c3c(-c5c6ccccc6c(-c6cccc7c6c6ccccc6n7-c6ccccc6)c6ccccc56)cccc34)c3ccccc23)cc1]

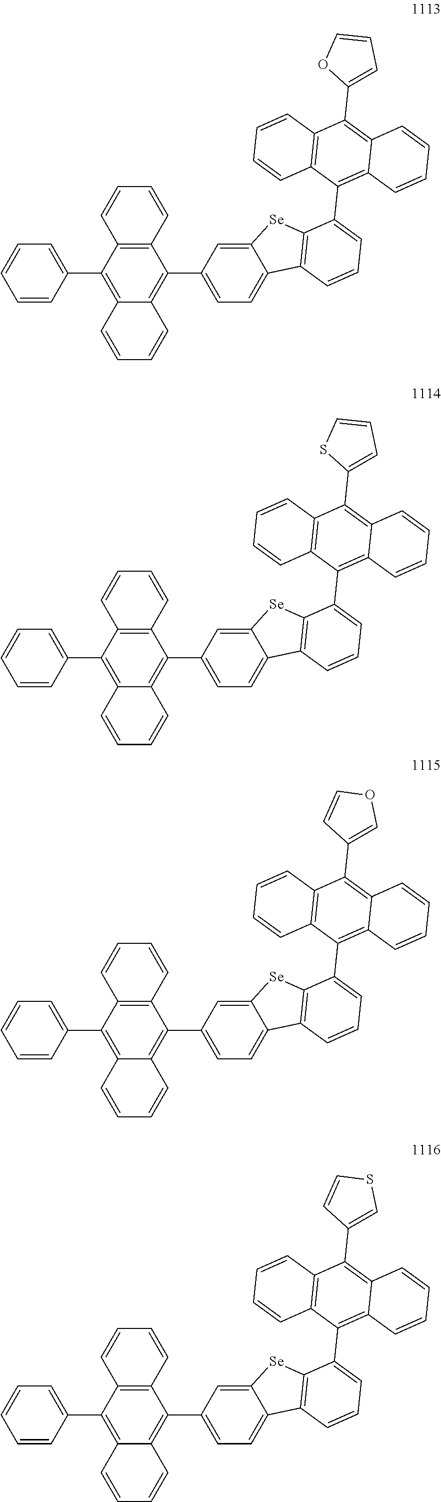 [SMILES: c1ccc(-c2c3ccccc3c(-c3ccc4c(c3)[se]c3c(-c5c6ccccc6c(-c6ccco6)c6ccccc56)cccc34)c3ccccc23)cc1 ; c1ccc(-c2c3ccccc3c(-c3ccc4c(c3)[se]c3c(-c5c6ccccc6c(-c6cccs6)c6ccccc56)cccc34)c3ccccc23)cc1 ; c1ccc(-c2c3ccccc3c(-c3ccc4c(c3)[se]c3c(-c5c6ccccc6c(-c6ccoc6)c6ccccc56)cccc34)c3ccccc23)cc1 ; c1ccc(-c2c3ccccc3c(-c3ccc4c(c3)[se]c3c(-c5c6ccccc6c(-c6ccsc6)c6ccccc56)cccc34)c3ccccc23)cc1]

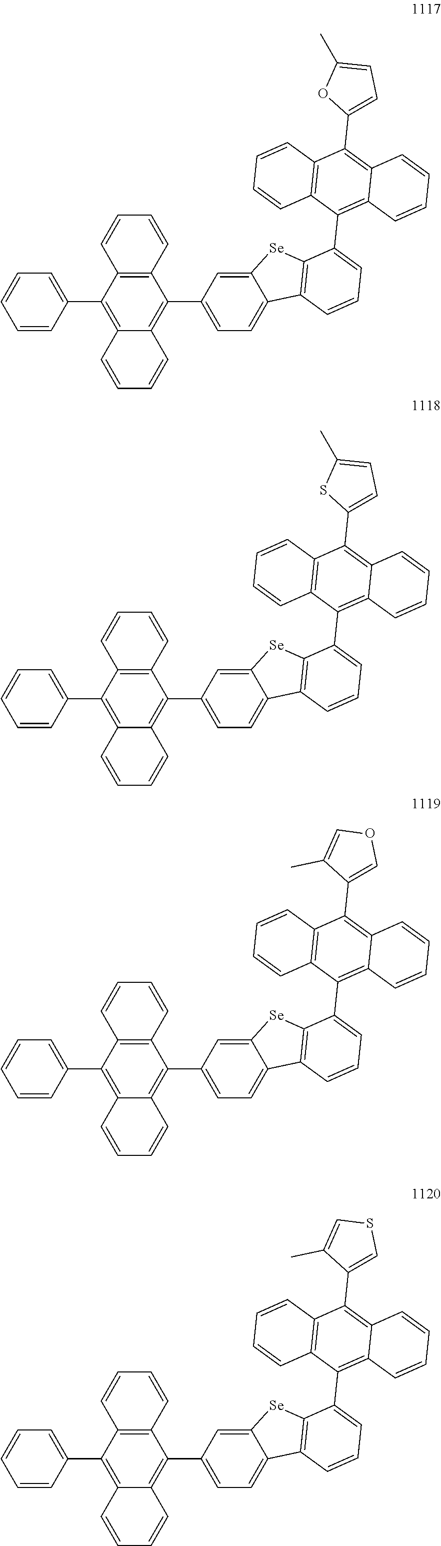 [SMILES: Cc1ccc(-c2c3ccccc3c(-c3cccc4c3[se]c3cc(-c5c6ccccc6c(-c6ccccc6)c6ccccc56)ccc34)c3ccccc23)o1 ; Cc1ccc(-c2c3ccccc3c(-c3cccc4c3[se]c3cc(-c5c6ccccc6c(-c6ccccc6)c6ccccc56)ccc34)c3ccccc23)s1 ; Cc1cocc1-c1c2ccccc2c(-c2cccc3c2[se]c2cc(-c4c5ccccc5c(-c5ccccc5)c5ccccc45)ccc23)c2ccccc12 ; Cc1cscc1-c1c2ccccc2c(-c2cccc3c2[se]c2cc(-c4c5ccccc5c(-c5ccccc5)c5ccccc45)ccc23)c2ccccc12]

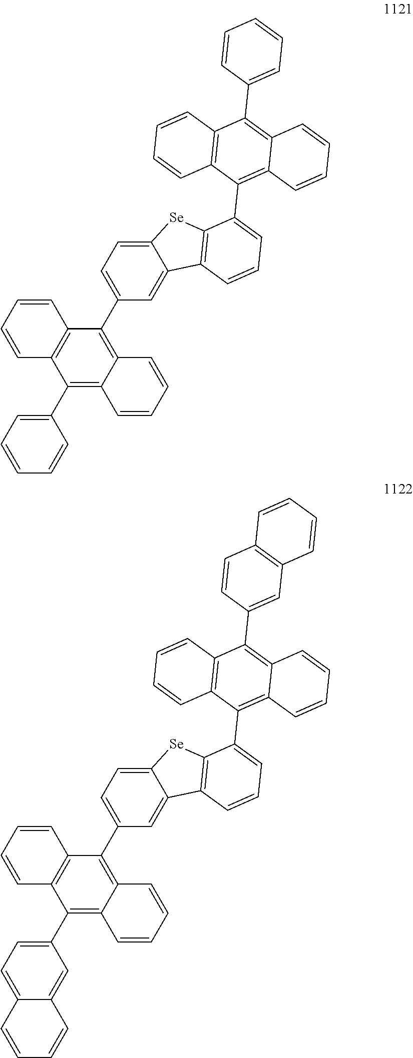 [SMILES: c1ccc(-c2c3ccccc3c(-c3ccc4[se]c5c(-c6c7ccccc7c(-c7ccccc7)c7ccccc67)cccc5c4c3)c3ccccc23)cc1 ; c1ccc2cc(-c3c4ccccc4c(-c4ccc5[se]c6c(-c7c8ccccc8c(-c8ccc9ccccc9c8)c8ccccc78)cccc6c5c4)c4ccccc34)ccc2c1]

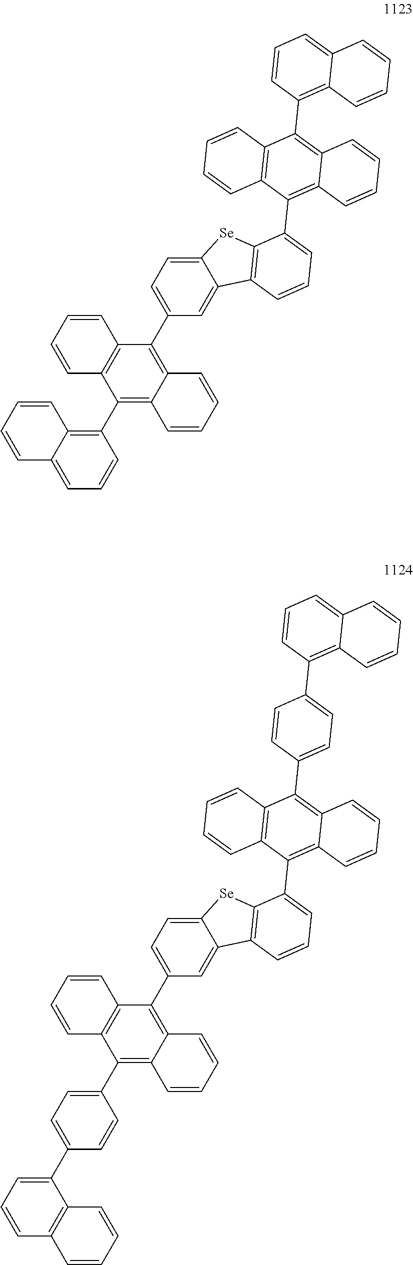 [SMILES: c1ccc2c(-c3c4ccccc4c(-c4ccc5[se]c6c(-c7c8ccccc8c(-c8cccc9ccccc89)c8ccccc78)cccc6c5c4)c4ccccc34)cccc2c1 ; c1ccc2c(-c3ccc(-c4c5ccccc5c(-c5ccc6[se]c7c(-c8c9ccccc9c(-c9ccc(-c%10cccc%11ccccc%10%11)cc9)c9ccccc89)cccc7c6c5)c5ccccc45)cc3)cccc2c1]

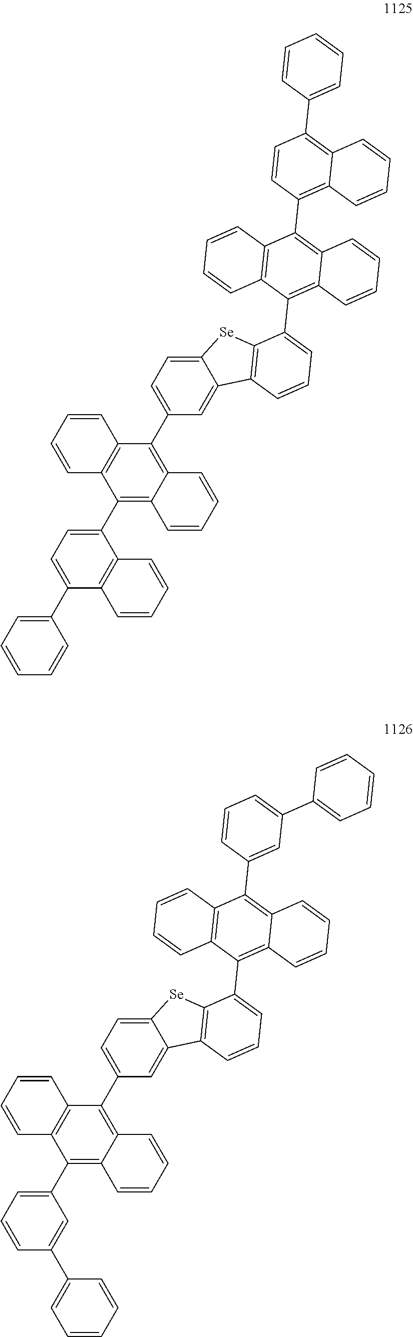 [SMILES: c1ccc(-c2ccc(-c3c4ccccc4c(-c4ccc5[se]c6c(-c7c8ccccc8c(-c8ccc(-c9ccccc9)c9ccccc89)c8ccccc78)cccc6c5c4)c4ccccc34)c3ccccc23)cc1 ; c1ccc(-c2cccc(-c3c4ccccc4c(-c4ccc5[se]c6c(-c7c8ccccc8c(-c8cccc(-c9ccccc9)c8)c8ccccc78)cccc6c5c4)c4ccccc34)c2)cc1]

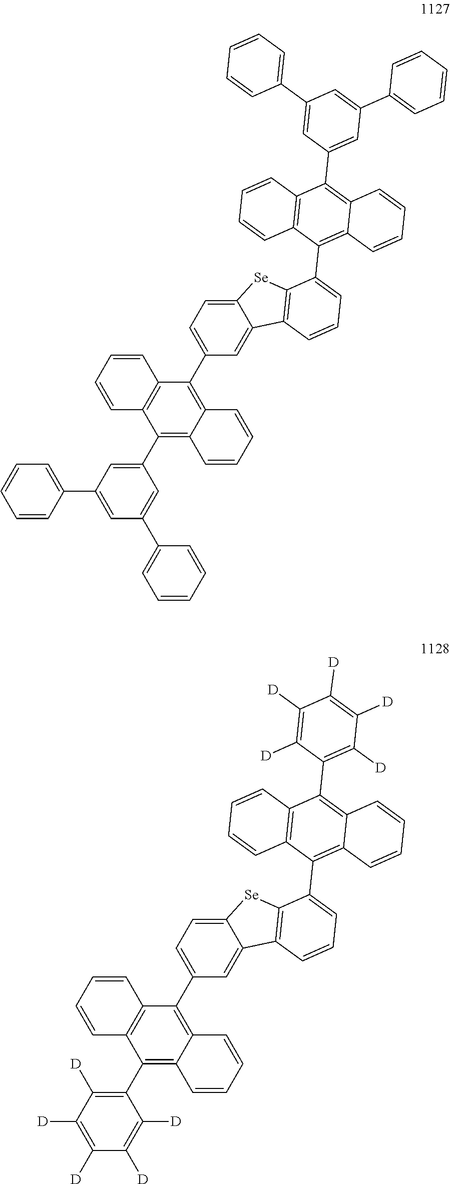 [SMILES: c1ccc(-c2cc(-c3ccccc3)cc(-c3c4ccccc4c(-c4ccc5[se]c6c(-c7c8ccccc8c(-c8cc(-c9ccccc9)cc(-c9ccccc9)c8)c8ccccc78)cccc6c5c4)c4ccccc34)c2)cc1 ; [2H]c1c([2H])c([2H])c(-c2c3ccccc3c(-c3ccc4[se]c5c(-c6c7ccccc7c(-c7c([2H])c([2H])c([2H])c([2H])c7[2H])c7ccccc67)cccc5c4c3)c3ccccc23)c([2H])c1[2H]]

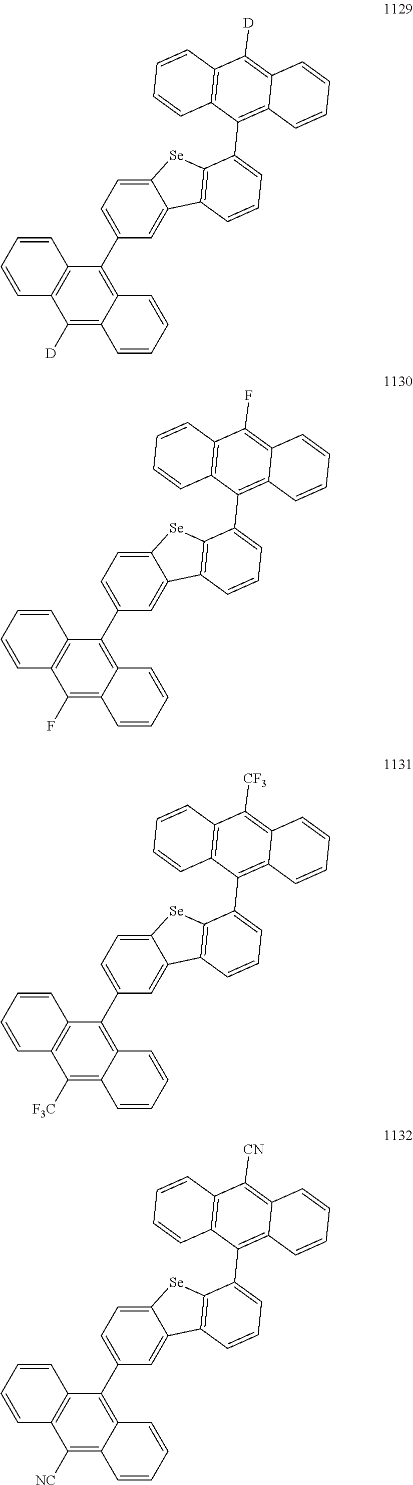 [SMILES: [2H]c1c2ccccc2c(-c2ccc3[se]c4c(-c5c6ccccc6c([2H])c6ccccc56)cccc4c3c2)c2ccccc12 ; Fc1c2ccccc2c(-c2ccc3[se]c4c(-c5c6ccccc6c(F)c6ccccc56)cccc4c3c2)c2ccccc12 ; FC(F)(F)c1c2ccccc2c(-c2ccc3[se]c4c(-c5c6ccccc6c(C(F)(F)F)c6ccccc56)cccc4c3c2)c2ccccc12 ; N#Cc1c2ccccc2c(-c2ccc3[se]c4c(-c5c6ccccc6c(C#N)c6ccccc56)cccc4c3c2)c2ccccc12]

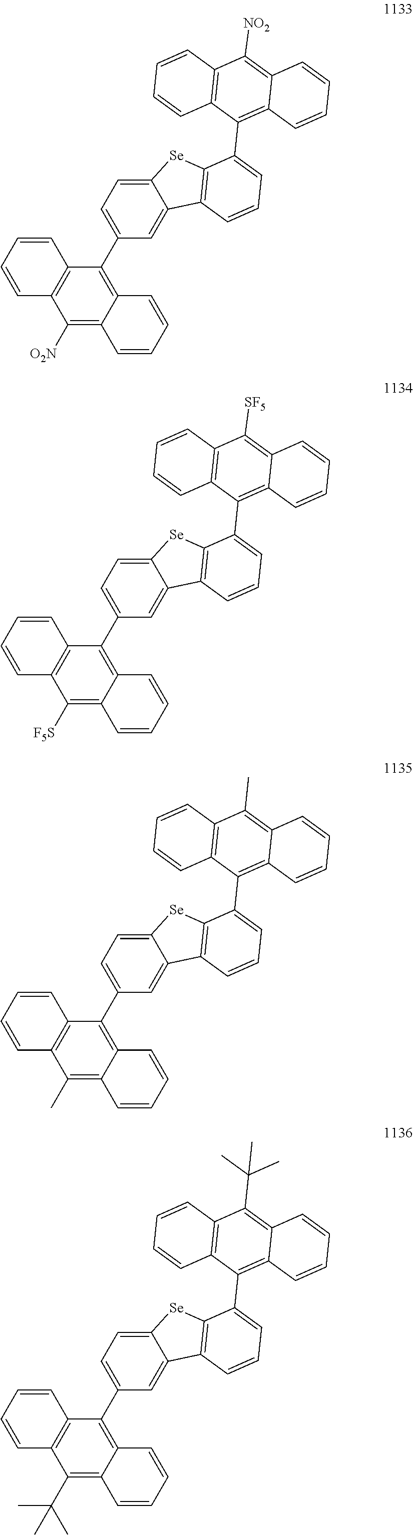

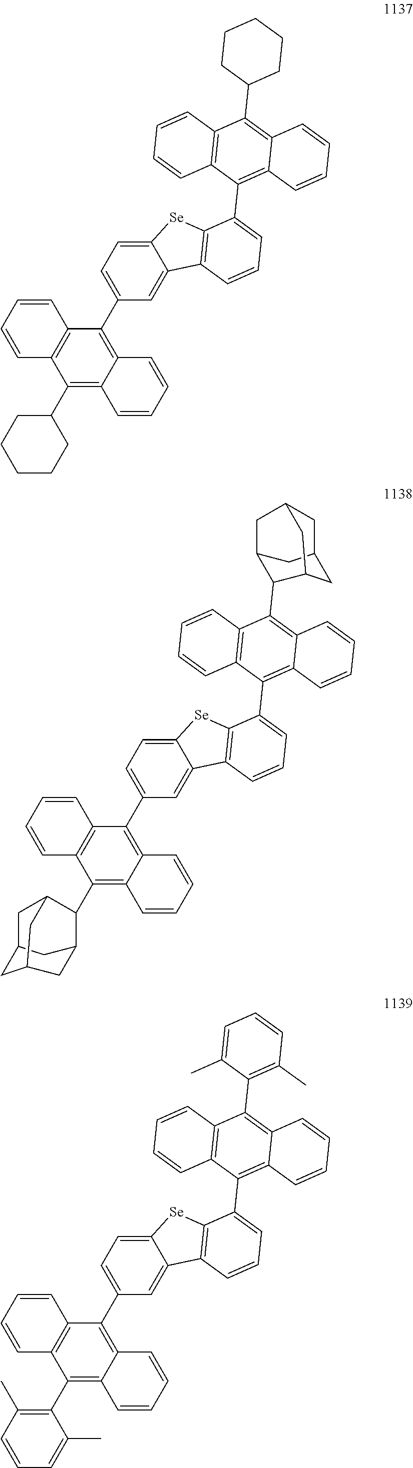 [SMILES: c1ccc2c(C3CCCCC3)c3ccccc3c(-c3ccc4[se]c5c(-c6c7ccccc7c(C7CCCCC7)c7ccccc67)cccc5c4c3)c2c1 ; c1ccc2c(C3C4CC5CC(C4)CC3C5)c3ccccc3c(-c3ccc4[se]c5c(-c6c7ccccc7c(C7C8CC9CC(C8)CC7C9)c7ccccc67)cccc5c4c3)c2c1 ; Cc1cccc(C)c1-c1c2ccccc2c(-c2ccc3[se]c4c(-c5c6ccccc6c(-c6c(C)cccc6C)c6ccccc56)cccc4c3c2)c2ccccc12]

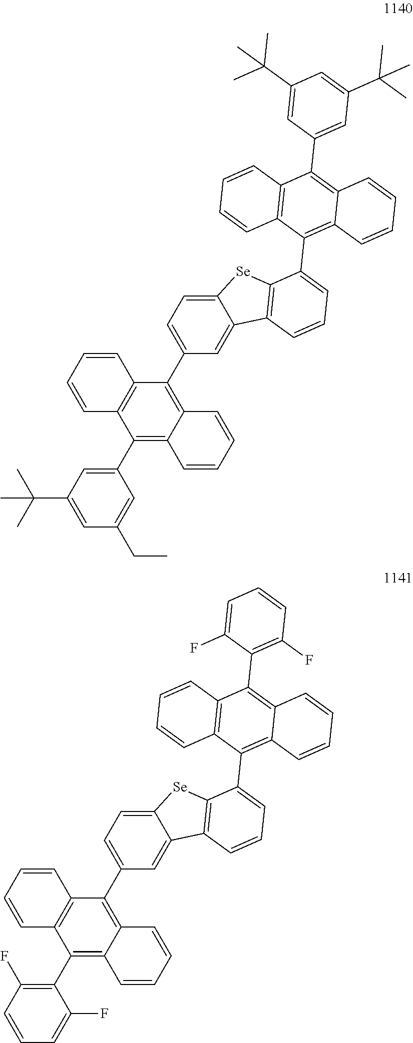 [SMILES: CCc1cc(-c2c3ccccc3c(-c3ccc4[se]c5c(-c6c7ccccc7c(-c7cc(C(C)(C)C)cc(C(C)(C)C)c7)c7ccccc67)cccc5c4c3)c3ccccc23)cc(C(C)(C)C)c1 ; Fc1cccc(F)c1-c1c2ccccc2c(-c2ccc3[se]c4c(-c5c6ccccc6c(-c6c(F)cccc6F)c6ccccc56)cccc4c3c2)c2ccccc12]

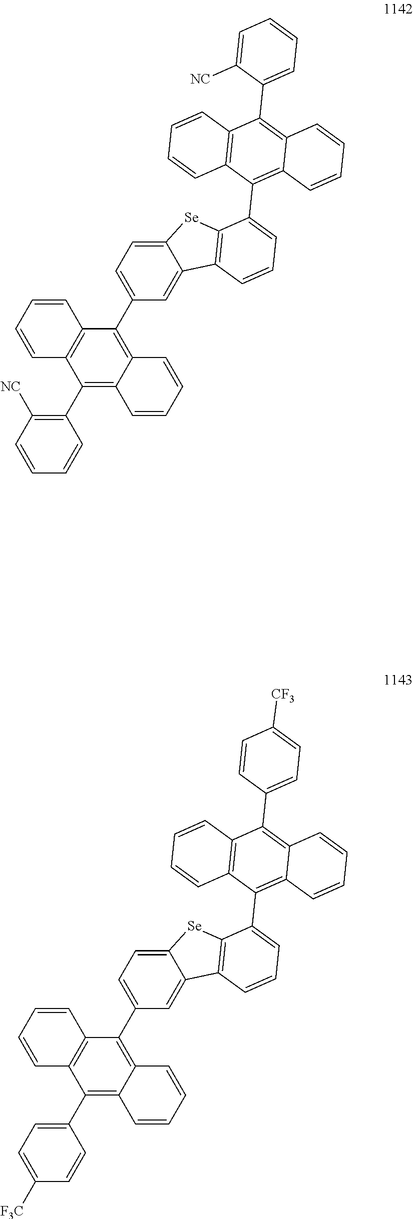 [SMILES: N#Cc1ccccc1-c1c2ccccc2c(-c2ccc3[se]c4c(-c5c6ccccc6c(-c6ccccc6C#N)c6ccccc56)cccc4c3c2)c2ccccc12 ; FC(F)(F)c1ccc(-c2c3ccccc3c(-c3ccc4[se]c5c(-c6c7ccccc7c(-c7ccc(C(F)(F)F)cc7)c7ccccc67)cccc5c4c3)c3ccccc23)cc1]

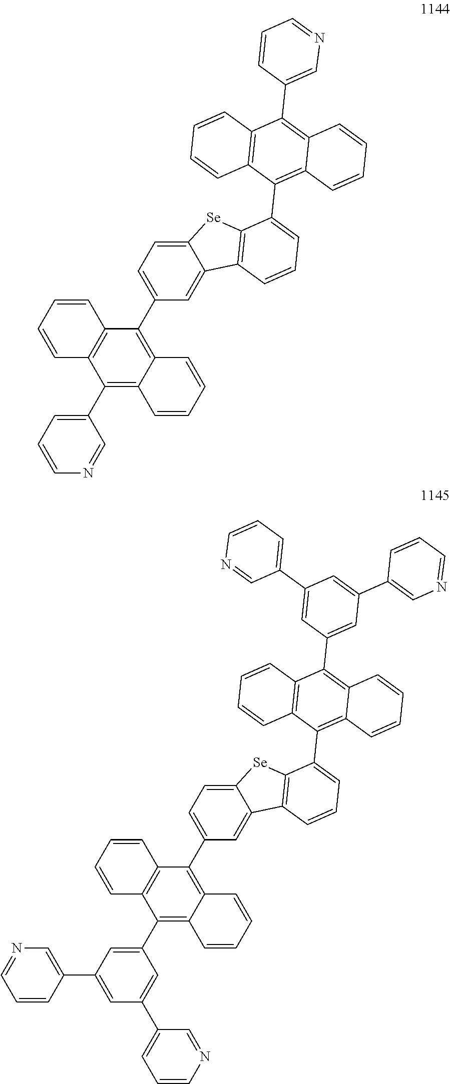 [SMILES: c1cncc(-c2c3ccccc3c(-c3ccc4[se]c5c(-c6c7ccccc7c(-c7cccnc7)c7ccccc67)cccc5c4c3)c3ccccc23)c1 ; c1cncc(-c2cc(-c3cccnc3)cc(-c3c4ccccc4c(-c4ccc5[se]c6c(-c7c8ccccc8c(-c8cc(-c9cccnc9)cc(-c9cccnc9)c8)c8ccccc78)cccc6c5c4)c4ccccc34)c2)c1]

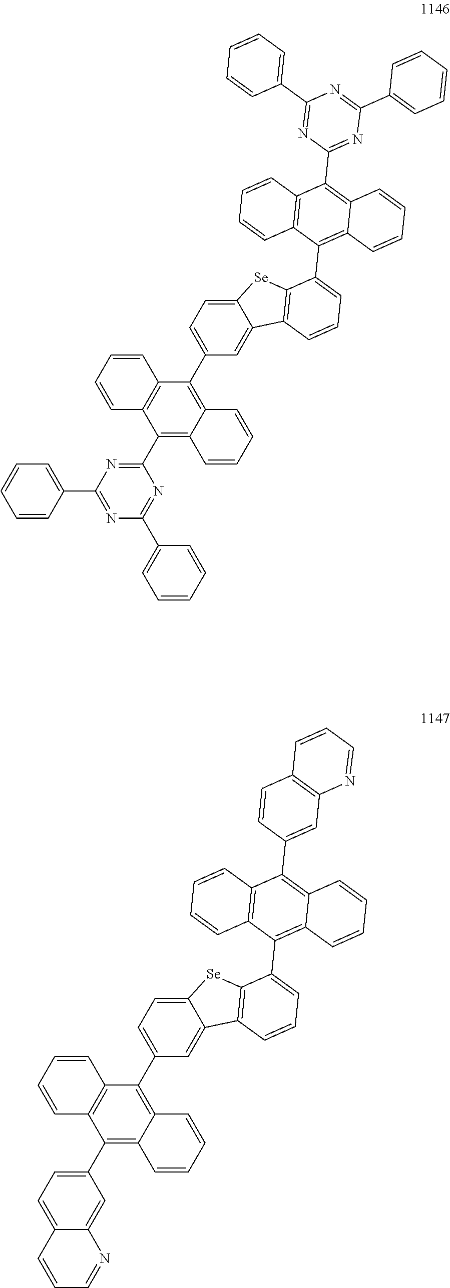 [SMILES: c1ccc(-c2nc(-c3ccccc3)nc(-c3c4ccccc4c(-c4ccc5[se]c6c(-c7c8ccccc8c(-c8nc(-c9ccccc9)nc(-c9ccccc9)n8)c8ccccc78)cccc6c5c4)c4ccccc34)n2)cc1 ; c1cnc2cc(-c3c4ccccc4c(-c4ccc5[se]c6c(-c7c8ccccc8c(-c8ccc9cccnc9c8)c8ccccc78)cccc6c5c4)c4ccccc34)ccc2c1]

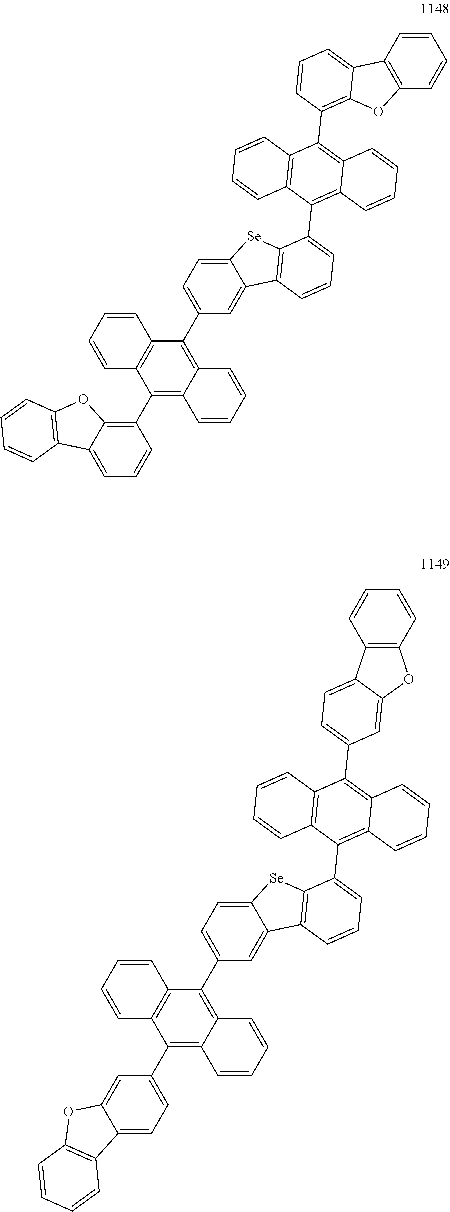 [SMILES: c1ccc2c(c1)oc1c(-c3c4ccccc4c(-c4ccc5[se]c6c(-c7c8ccccc8c(-c8cccc9c8oc8ccccc89)c8ccccc78)cccc6c5c4)c4ccccc34)cccc12 ; c1ccc2c(c1)oc1cc(-c3c4ccccc4c(-c4ccc5[se]c6c(-c7c8ccccc8c(-c8ccc9c(c8)oc8ccccc89)c8ccccc78)cccc6c5c4)c4ccccc34)ccc12]

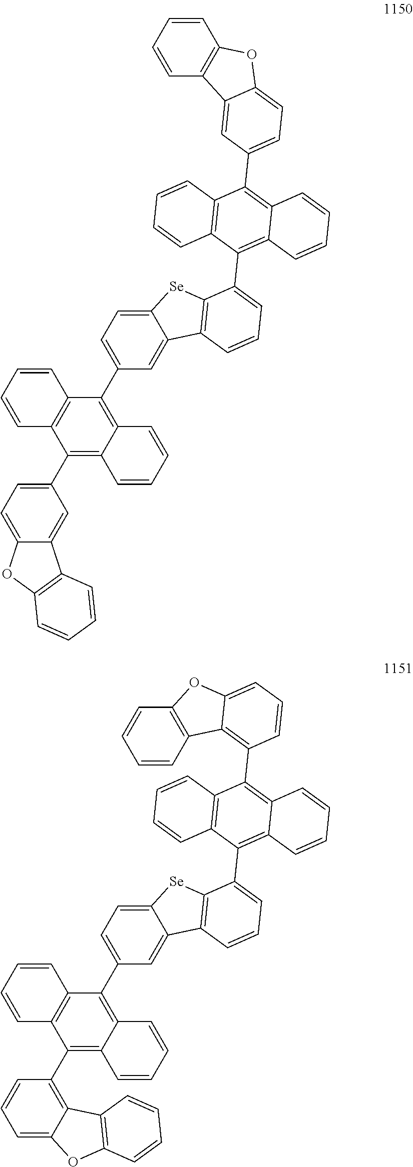 [SMILES: c1ccc2c(c1)oc1ccc(-c3c4ccccc4c(-c4ccc5[se]c6c(-c7c8ccccc8c(-c8ccc9oc%10ccccc%10c9c8)c8ccccc78)cccc6c5c4)c4ccccc34)cc12 ; c1ccc2c(c1)oc1cccc(-c3c4ccccc4c(-c4ccc5[se]c6c(-c7c8ccccc8c(-c8cccc9oc%10ccccc%10c89)c8ccccc78)cccc6c5c4)c4ccccc34)c12]

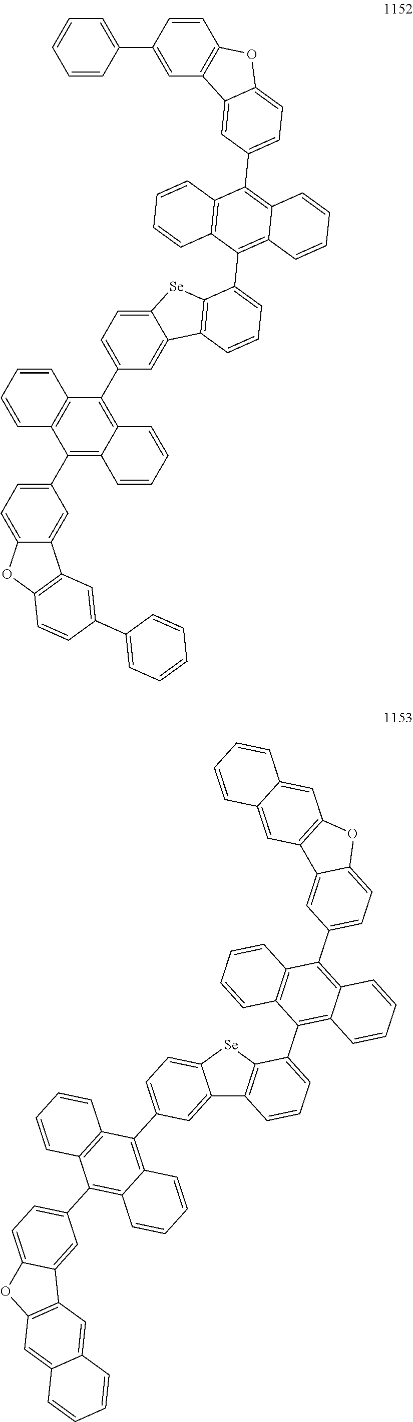 [SMILES: c1ccc(-c2ccc3oc4ccc(-c5c6ccccc6c(-c6ccc7[se]c8c(-c9c%10ccccc%10c(-c%10ccc%11oc%12ccc(-c%13ccccc%13)cc%12c%11c%10)c%10ccccc9%10)cccc8c7c6)c6ccccc56)cc4c3c2)cc1 ; c1ccc2cc3c(cc2c1)oc1ccc(-c2c4ccccc4c(-c4ccc5[se]c6c(-c7c8ccccc8c(-c8ccc9oc%10cc%11ccccc%11cc%10c9c8)c8ccccc78)cccc6c5c4)c4ccccc24)cc13]

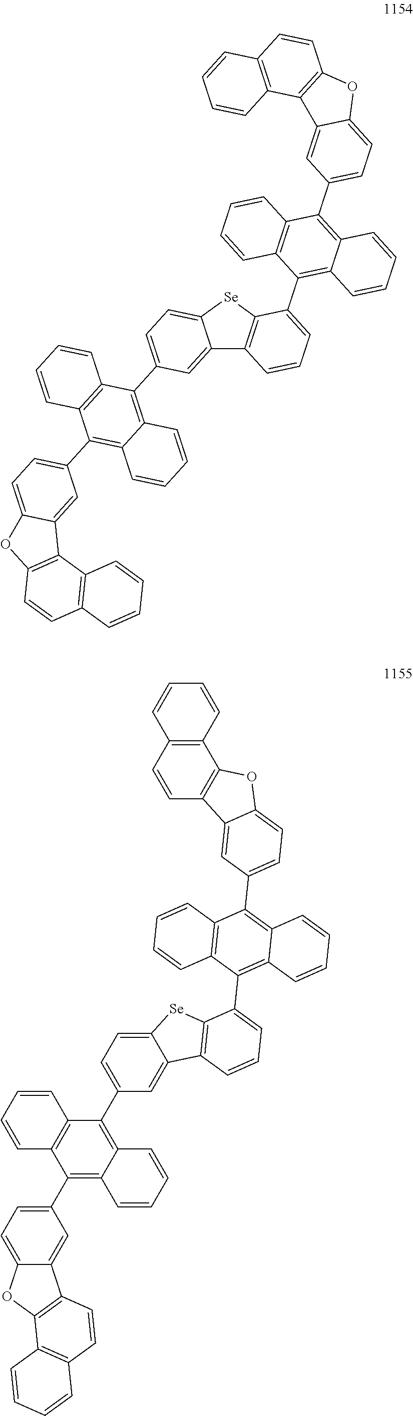 [SMILES: c1ccc2c(c1)ccc1oc3ccc(-c4c5ccccc5c(-c5ccc6[se]c7c(-c8c9ccccc9c(-c9ccc%10oc%11ccc%12ccccc%12c%11c%10c9)c9ccccc89)cccc7c6c5)c5ccccc45)cc3c12 ; c1ccc2c(c1)ccc1c3cc(-c4c5ccccc5c(-c5ccc6[se]c7c(-c8c9ccccc9c(-c9ccc%10oc%11c%12ccccc%12ccc%11c%10c9)c9ccccc89)cccc7c6c5)c5ccccc45)ccc3oc21]

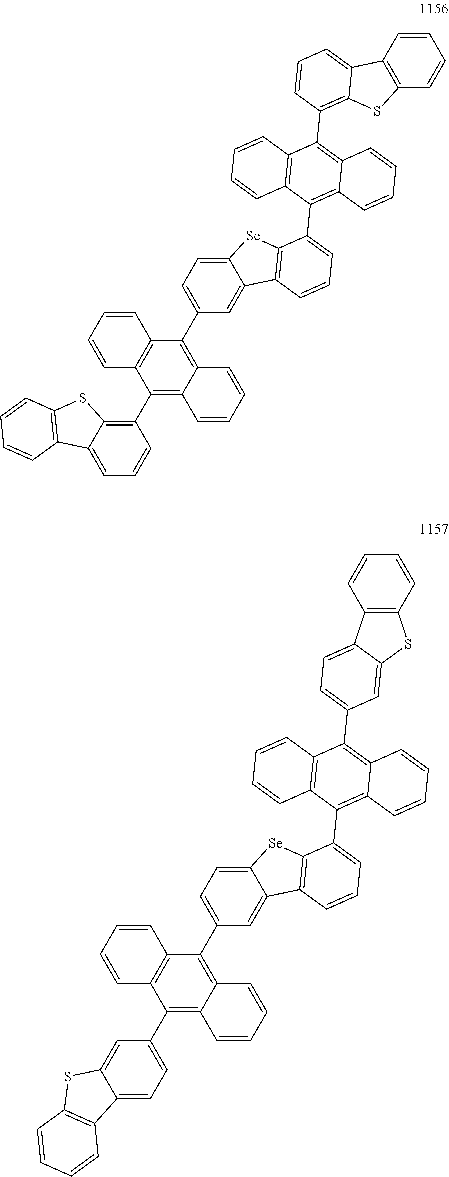 [SMILES: c1ccc2c(c1)sc1c(-c3c4ccccc4c(-c4ccc5[se]c6c(-c7c8ccccc8c(-c8cccc9c8sc8ccccc89)c8ccccc78)cccc6c5c4)c4ccccc34)cccc12 ; c1ccc2c(c1)sc1cc(-c3c4ccccc4c(-c4ccc5[se]c6c(-c7c8ccccc8c(-c8ccc9c(c8)sc8ccccc89)c8ccccc78)cccc6c5c4)c4ccccc34)ccc12]

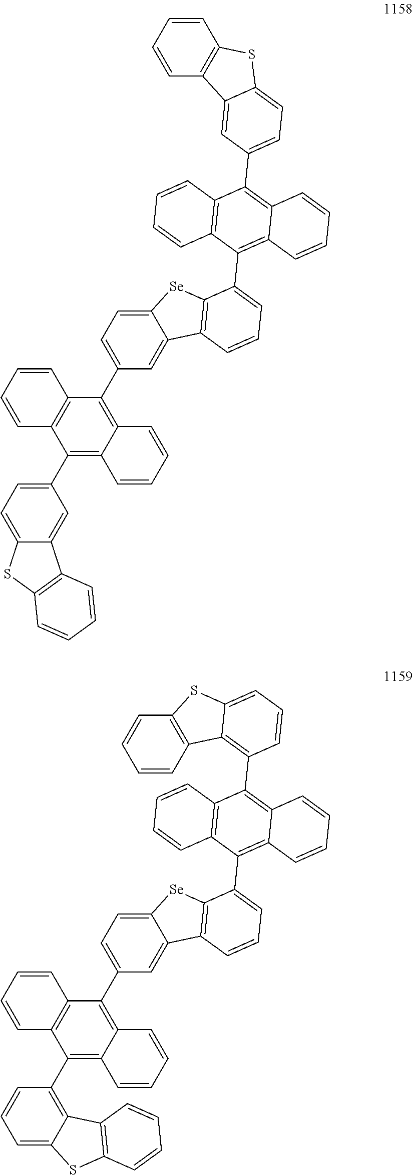 [SMILES: c1ccc2c(c1)sc1ccc(-c3c4ccccc4c(-c4ccc5[se]c6c(-c7c8ccccc8c(-c8ccc9sc%10ccccc%10c9c8)c8ccccc78)cccc6c5c4)c4ccccc34)cc12 ; c1ccc2c(c1)sc1cccc(-c3c4ccccc4c(-c4ccc5[se]c6c(-c7c8ccccc8c(-c8cccc9sc%10ccccc%10c89)c8ccccc78)cccc6c5c4)c4ccccc34)c12]

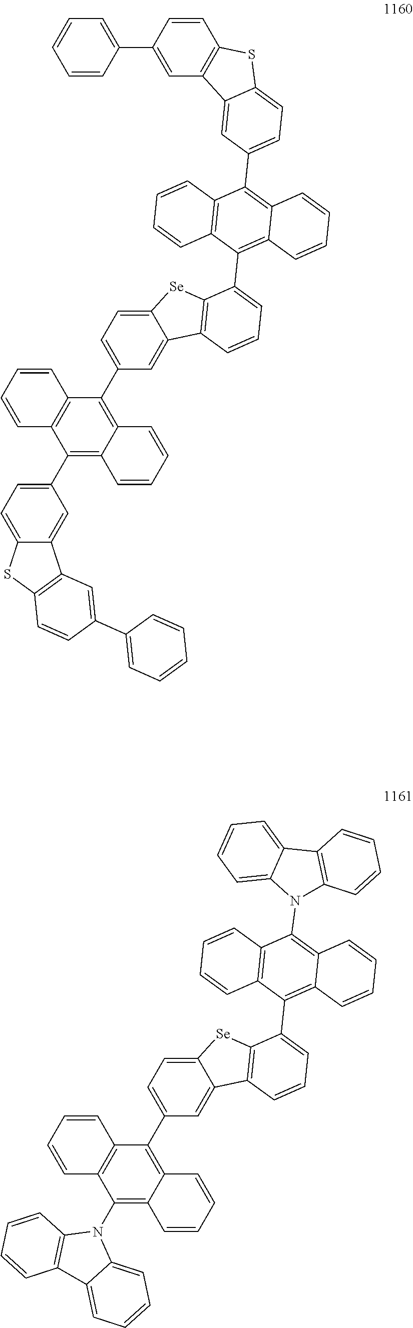 [SMILES: c1ccc(-c2ccc3sc4ccc(-c5c6ccccc6c(-c6ccc7[se]c8c(-c9c%10ccccc%10c(-c%10ccc%11sc%12ccc(-c%13ccccc%13)cc%12c%11c%10)c%10ccccc9%10)cccc8c7c6)c6ccccc56)cc4c3c2)cc1 ; c1ccc2c(-n3c4ccccc4c4ccccc43)c3ccccc3c(-c3ccc4[se]c5c(-c6c7ccccc7c(-n7c8ccccc8c8ccccc87)c7ccccc67)cccc5c4c3)c2c1]

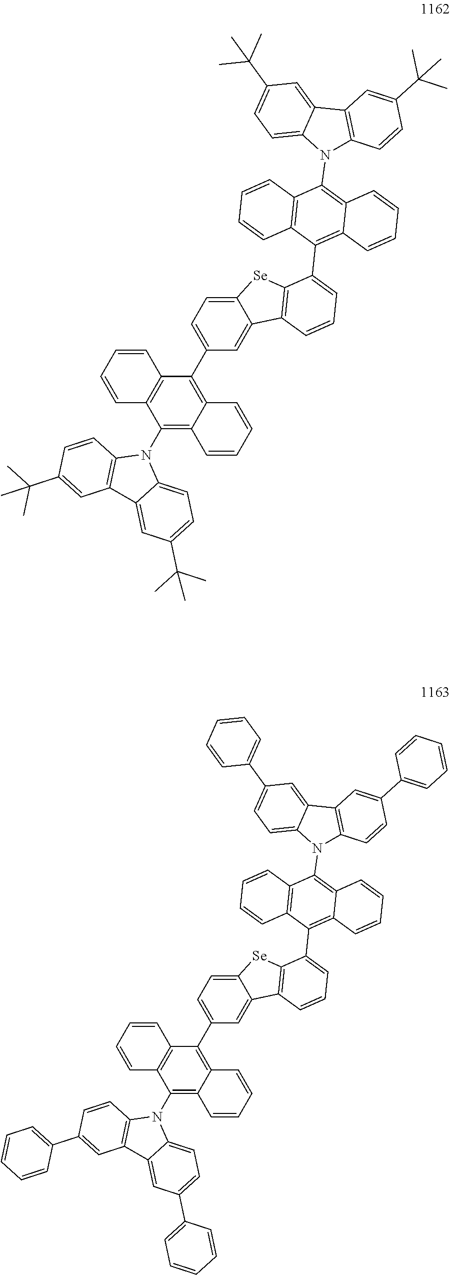 [SMILES: CC(C)(C)c1ccc2c(c1)c1cc(C(C)(C)C)ccc1n2-c1c2ccccc2c(-c2ccc3[se]c4c(-c5c6ccccc6c(-n6c7ccc(C(C)(C)C)cc7c7cc(C(C)(C)C)ccc76)c6ccccc56)cccc4c3c2)c2ccccc12 ; c1ccc(-c2ccc3c(c2)c2cc(-c4ccccc4)ccc2n3-c2c3ccccc3c(-c3ccc4[se]c5c(-c6c7ccccc7c(-n7c8ccc(-c9ccccc9)cc8c8cc(-c9ccccc9)ccc87)c7ccccc67)cccc5c4c3)c3ccccc23)cc1]

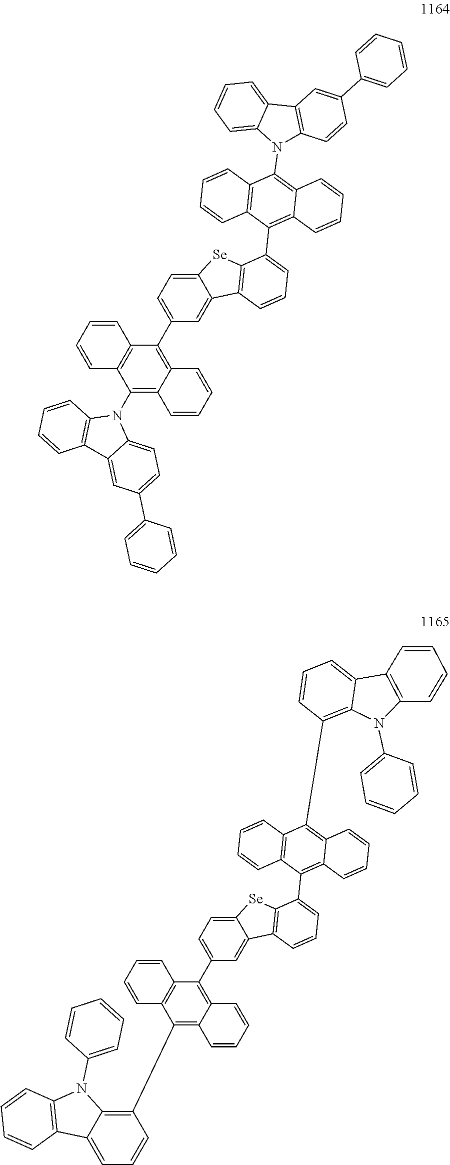 [SMILES: c1ccc(-c2ccc3c(c2)c2ccccc2n3-c2c3ccccc3c(-c3ccc4[se]c5c(-c6c7ccccc7c(-n7c8ccccc8c8cc(-c9ccccc9)ccc87)c7ccccc67)cccc5c4c3)c3ccccc23)cc1 ; c1ccc(-n2c3ccccc3c3cccc(-c4c5ccccc5c(-c5ccc6[se]c7c(-c8c9ccccc9c(-c9cccc%10c%11ccccc%11n(-c%11ccccc%11)c9%10)c9ccccc89)cccc7c6c5)c5ccccc45)c32)cc1]

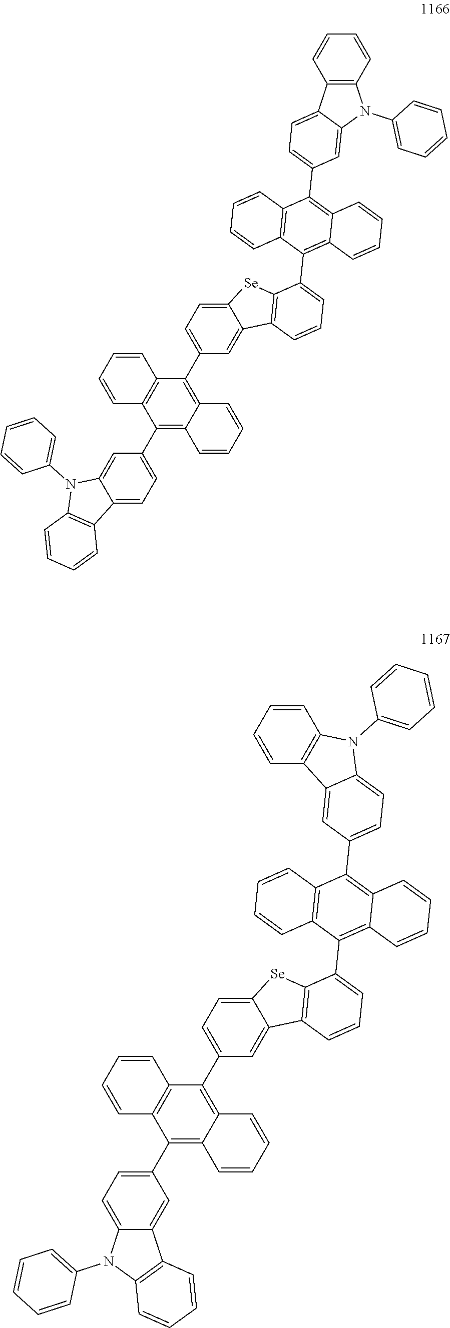 [SMILES: c1ccc(-n2c3ccccc3c3ccc(-c4c5ccccc5c(-c5ccc6[se]c7c(-c8c9ccccc9c(-c9ccc%10c%11ccccc%11n(-c%11ccccc%11)c%10c9)c9ccccc89)cccc7c6c5)c5ccccc45)cc32)cc1 ; c1ccc(-n2c3ccccc3c3cc(-c4c5ccccc5c(-c5ccc6[se]c7c(-c8c9ccccc9c(-c9ccc%10c(c9)c9ccccc9n%10-c9ccccc9)c9ccccc89)cccc7c6c5)c5ccccc45)ccc32)cc1]

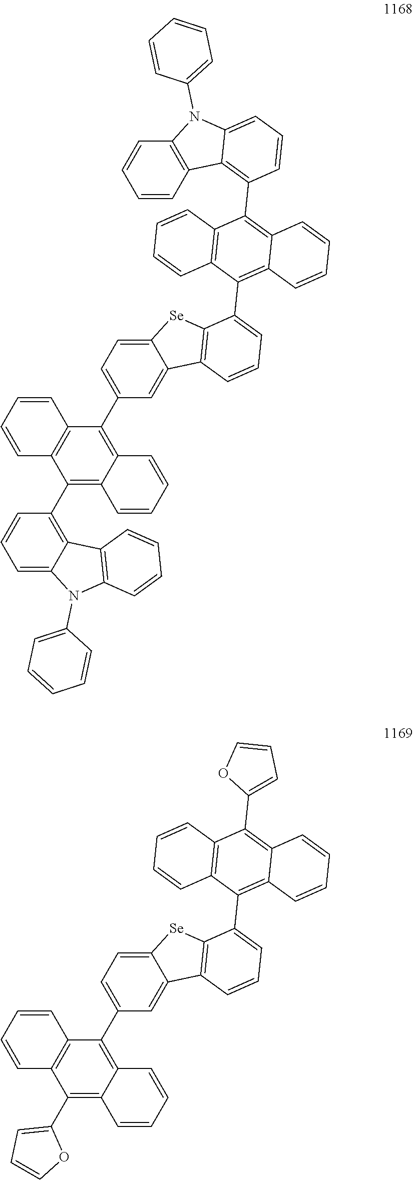 [SMILES: c1ccc(-n2c3ccccc3c3c(-c4c5ccccc5c(-c5ccc6[se]c7c(-c8c9ccccc9c(-c9cccc%10c9c9ccccc9n%10-c9ccccc9)c9ccccc89)cccc7c6c5)c5ccccc45)cccc32)cc1 ; c1coc(-c2c3ccccc3c(-c3ccc4[se]c5c(-c6c7ccccc7c(-c7ccco7)c7ccccc67)cccc5c4c3)c3ccccc23)c1]

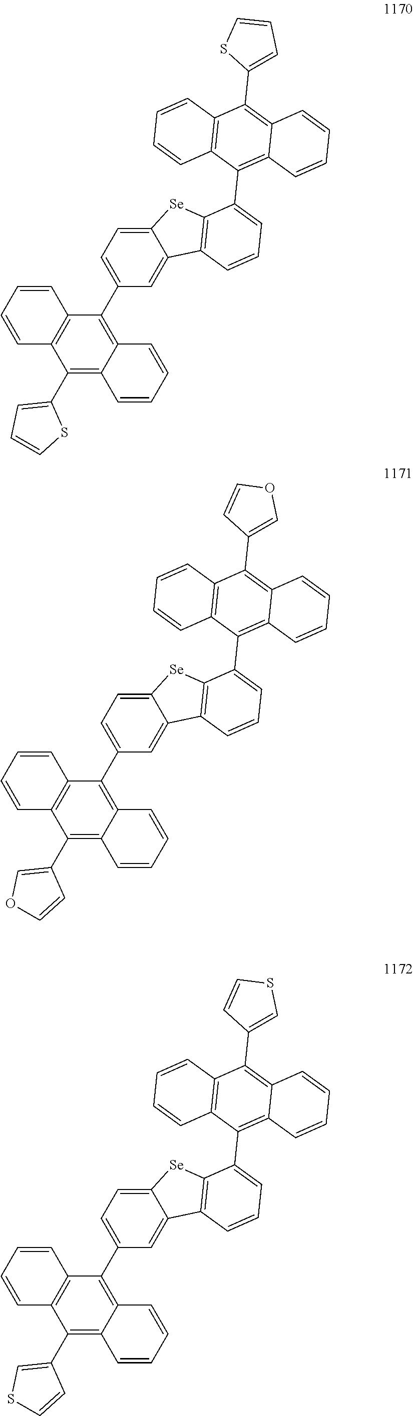 [SMILES: c1csc(-c2c3ccccc3c(-c3ccc4[se]c5c(-c6c7ccccc7c(-c7cccs7)c7ccccc67)cccc5c4c3)c3ccccc23)c1 ; c1ccc2c(-c3ccc4[se]c5c(-c6c7ccccc7c(-c7ccoc7)c7ccccc67)cccc5c4c3)c3ccccc3c(-c3ccoc3)c2c1 ; c1ccc2c(-c3ccc4[se]c5c(-c6c7ccccc7c(-c7ccsc7)c7ccccc67)cccc5c4c3)c3ccccc3c(-c3ccsc3)c2c1]

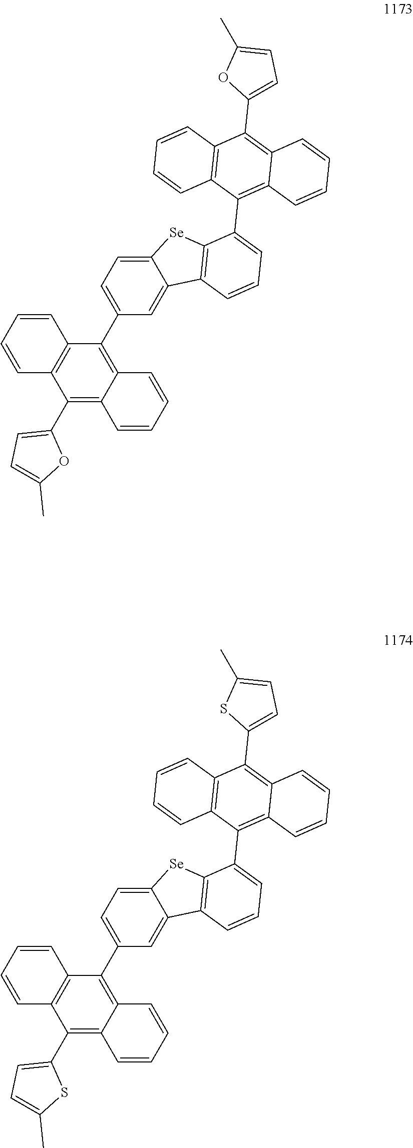 [SMILES: Cc1ccc(-c2c3ccccc3c(-c3ccc4[se]c5c(-c6c7ccccc7c(-c7ccc(C)o7)c7ccccc67)cccc5c4c3)c3ccccc23)o1 ; Cc1ccc(-c2c3ccccc3c(-c3ccc4[se]c5c(-c6c7ccccc7c(-c7ccc(C)s7)c7ccccc67)cccc5c4c3)c3ccccc23)s1]

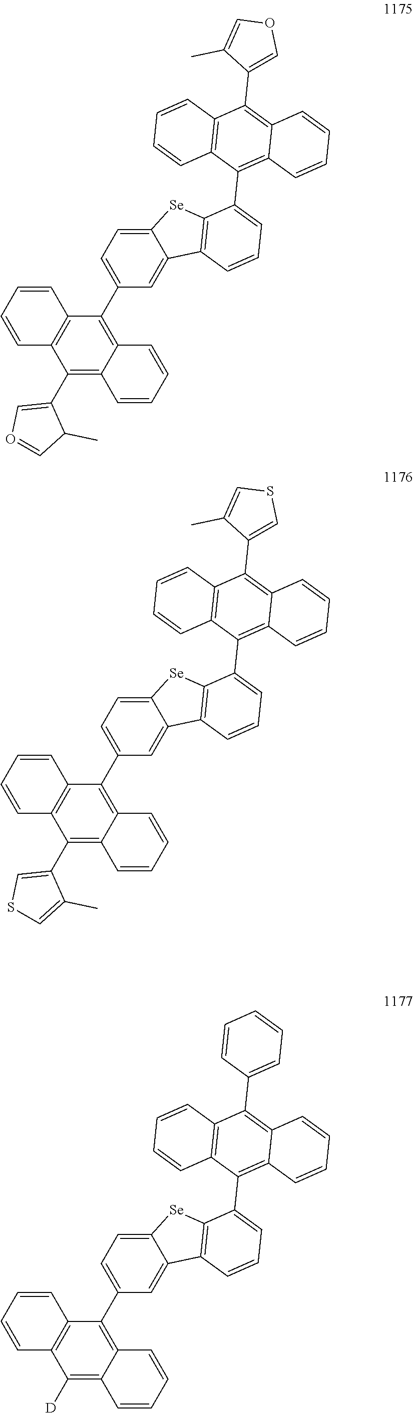

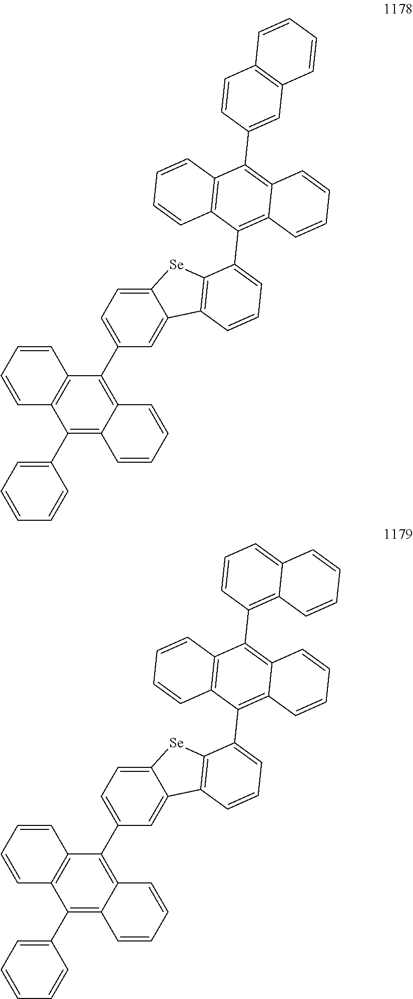 [SMILES: c1ccc(-c2c3ccccc3c(-c3ccc4[se]c5c(-c6c7ccccc7c(-c7ccc8ccccc8c7)c7ccccc67)cccc5c4c3)c3ccccc23)cc1 ; c1ccc(-c2c3ccccc3c(-c3ccc4[se]c5c(-c6c7ccccc7c(-c7cccc8ccccc78)c7ccccc67)cccc5c4c3)c3ccccc23)cc1]

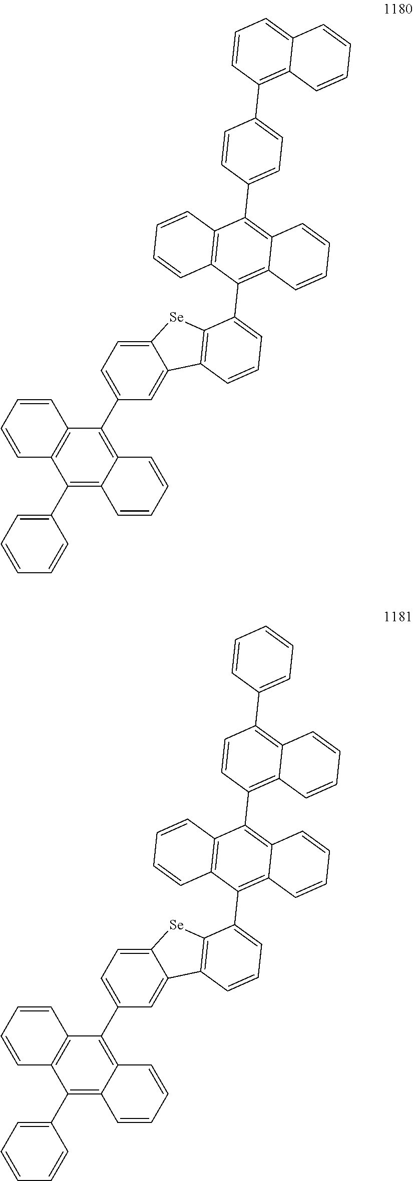 [SMILES: c1ccc(-c2c3ccccc3c(-c3ccc4[se]c5c(-c6c7ccccc7c(-c7ccc(-c8cccc9ccccc89)cc7)c7ccccc67)cccc5c4c3)c3ccccc23)cc1 ; c1ccc(-c2ccc(-c3c4ccccc4c(-c4cccc5c4[se]c4ccc(-c6c7ccccc7c(-c7ccccc7)c7ccccc67)cc45)c4ccccc34)c3ccccc23)cc1]

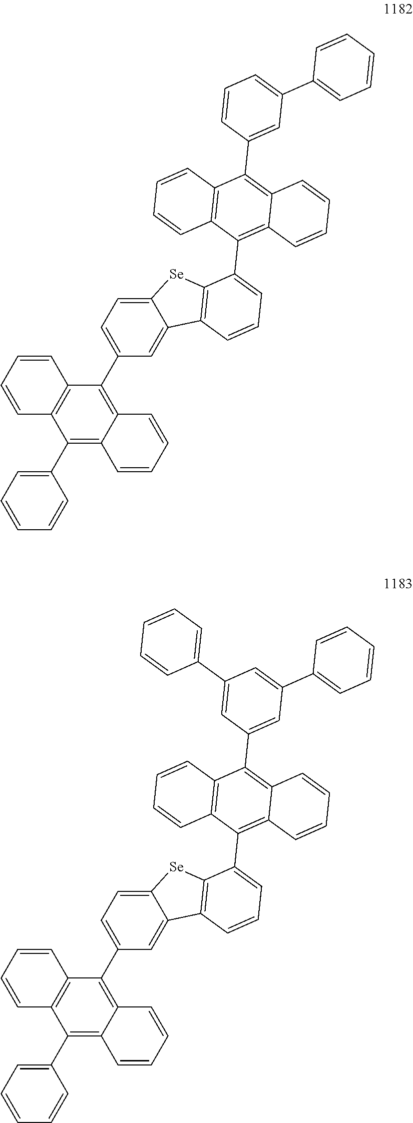 [SMILES: c1ccc(-c2cccc(-c3c4ccccc4c(-c4cccc5c4[se]c4ccc(-c6c7ccccc7c(-c7ccccc7)c7ccccc67)cc45)c4ccccc34)c2)cc1 ; c1ccc(-c2cc(-c3ccccc3)cc(-c3c4ccccc4c(-c4cccc5c4[se]c4ccc(-c6c7ccccc7c(-c7ccccc7)c7ccccc67)cc45)c4ccccc34)c2)cc1]

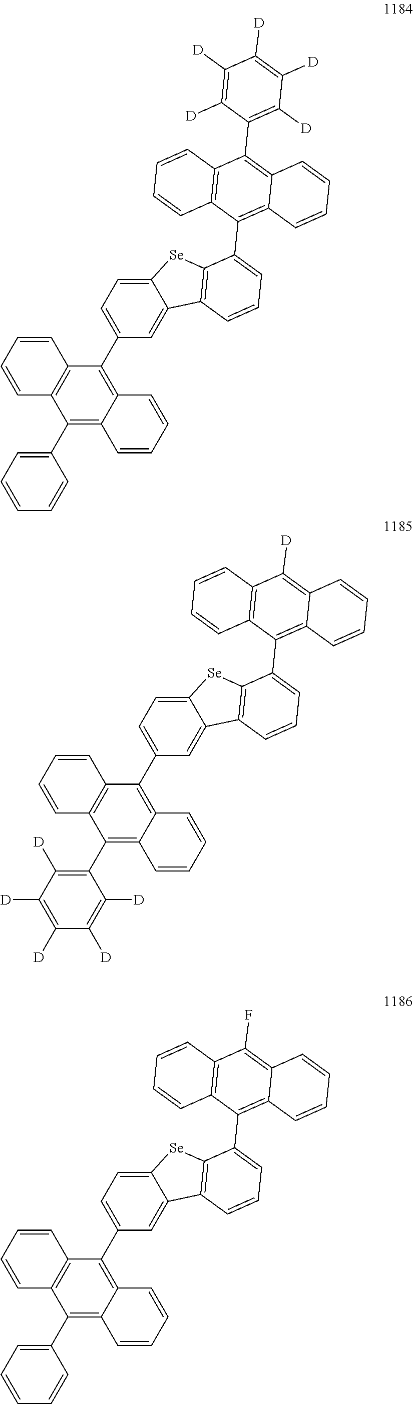 [SMILES: [2H]c1c([2H])c([2H])c(-c2c3ccccc3c(-c3cccc4c3[se]c3ccc(-c5c6ccccc6c(-c6ccccc6)c6ccccc56)cc34)c3ccccc23)c([2H])c1[2H] ; [2H]c1c([2H])c([2H])c(-c2c3ccccc3c(-c3ccc4[se]c5c(-c6c7ccccc7c([2H])c7ccccc67)cccc5c4c3)c3ccccc23)c([2H])c1[2H] ; Fc1c2ccccc2c(-c2cccc3c2[se]c2ccc(-c4c5ccccc5c(-c5ccccc5)c5ccccc45)cc23)c2ccccc12]

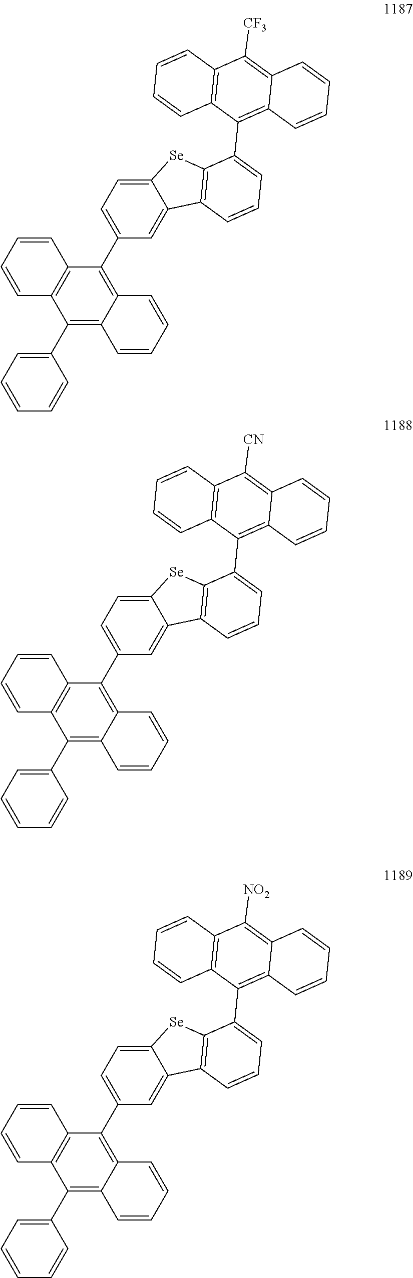 [SMILES: FC(F)(F)c1c2ccccc2c(-c2cccc3c2[se]c2ccc(-c4c5ccccc5c(-c5ccccc5)c5ccccc45)cc23)c2ccccc12 ; N#Cc1c2ccccc2c(-c2cccc3c2[se]c2ccc(-c4c5ccccc5c(-c5ccccc5)c5ccccc45)cc23)c2ccccc12 ; O=[N+]([O-])c1c2ccccc2c(-c2cccc3c2[se]c2ccc(-c4c5ccccc5c(-c5ccccc5)c5ccccc45)cc23)c2ccccc12]

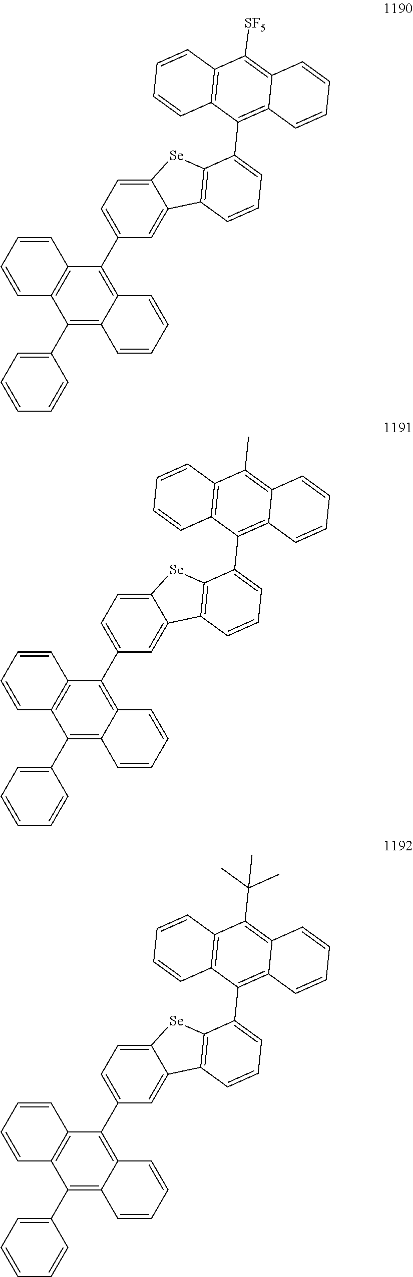

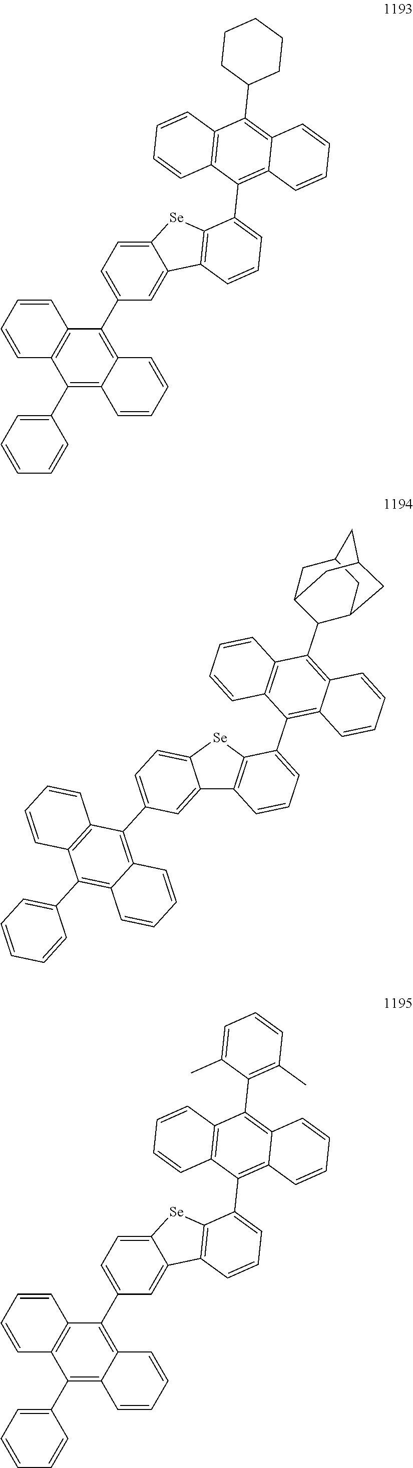 [SMILES: c1ccc(-c2c3ccccc3c(-c3ccc4[se]c5c(-c6c7ccccc7c(C7CCCCC7)c7ccccc67)cccc5c4c3)c3ccccc23)cc1 ; c1ccc(-c2c3ccccc3c(-c3ccc4[se]c5c(-c6c7ccccc7c(C7C8CC9CC(C8)CC7C9)c7ccccc67)cccc5c4c3)c3ccccc23)cc1 ; Cc1cccc(C)c1-c1c2ccccc2c(-c2cccc3c2[se]c2ccc(-c4c5ccccc5c(-c5ccccc5)c5ccccc45)cc23)c2ccccc12]

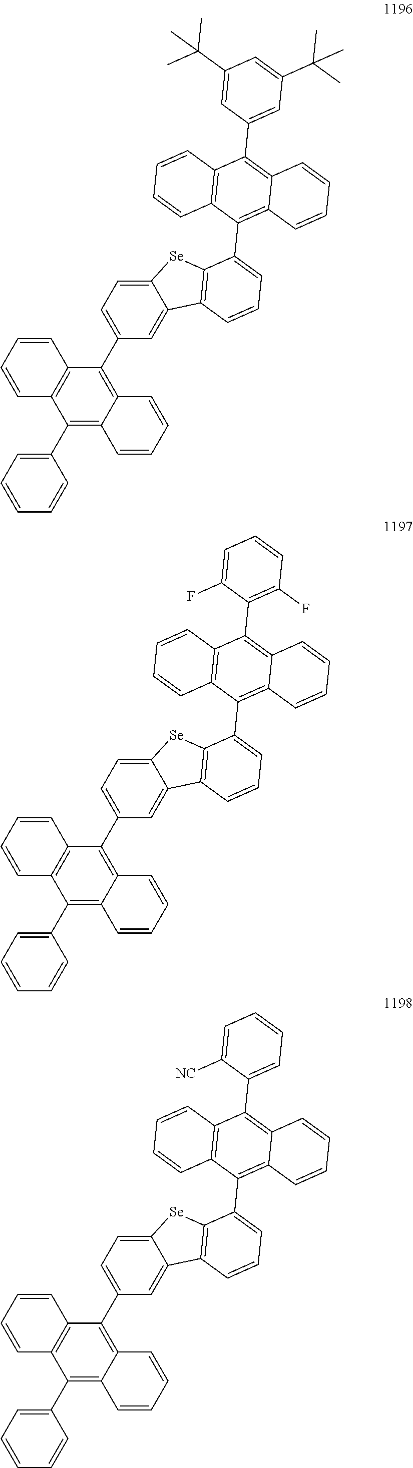 [SMILES: CC(C)(C)c1cc(-c2c3ccccc3c(-c3cccc4c3[se]c3ccc(-c5c6ccccc6c(-c6ccccc6)c6ccccc56)cc34)c3ccccc23)cc(C(C)(C)C)c1 ; Fc1cccc(F)c1-c1c2ccccc2c(-c2cccc3c2[se]c2ccc(-c4c5ccccc5c(-c5ccccc5)c5ccccc45)cc23)c2ccccc12 ; N#Cc1ccccc1-c1c2ccccc2c(-c2cccc3c2[se]c2ccc(-c4c5ccccc5c(-c5ccccc5)c5ccccc45)cc23)c2ccccc12]

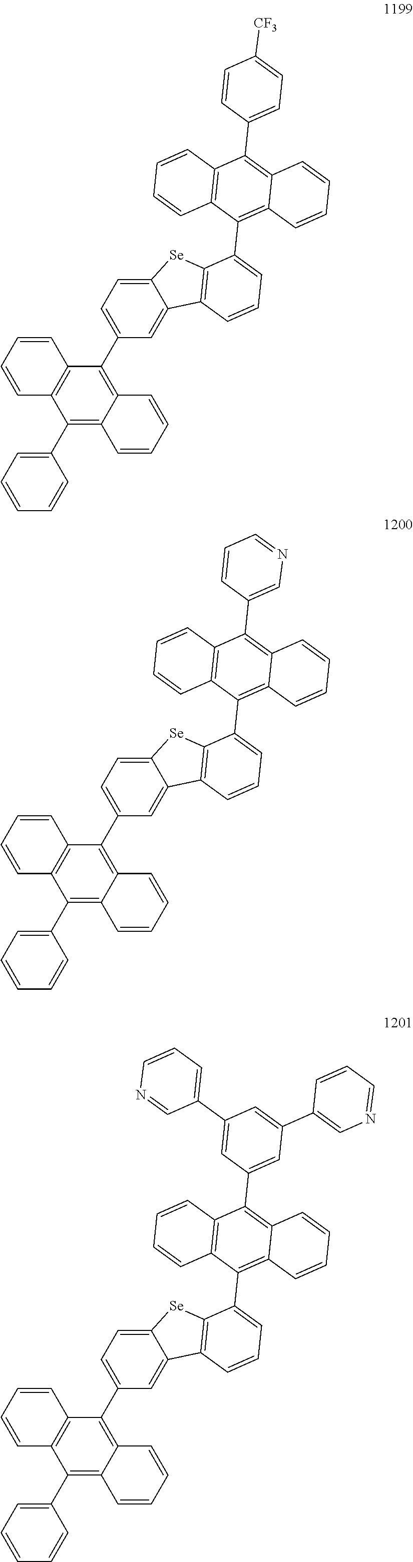 [SMILES: FC(F)(F)c1ccc(-c2c3ccccc3c(-c3cccc4c3[se]c3ccc(-c5c6ccccc6c(-c6ccccc6)c6ccccc56)cc34)c3ccccc23)cc1 ; c1ccc(-c2c3ccccc3c(-c3ccc4[se]c5c(-c6c7ccccc7c(-c7cccnc7)c7ccccc67)cccc5c4c3)c3ccccc23)cc1 ; c1ccc(-c2c3ccccc3c(-c3ccc4[se]c5c(-c6c7ccccc7c(-c7cc(-c8cccnc8)cc(-c8cccnc8)c7)c7ccccc67)cccc5c4c3)c3ccccc23)cc1]

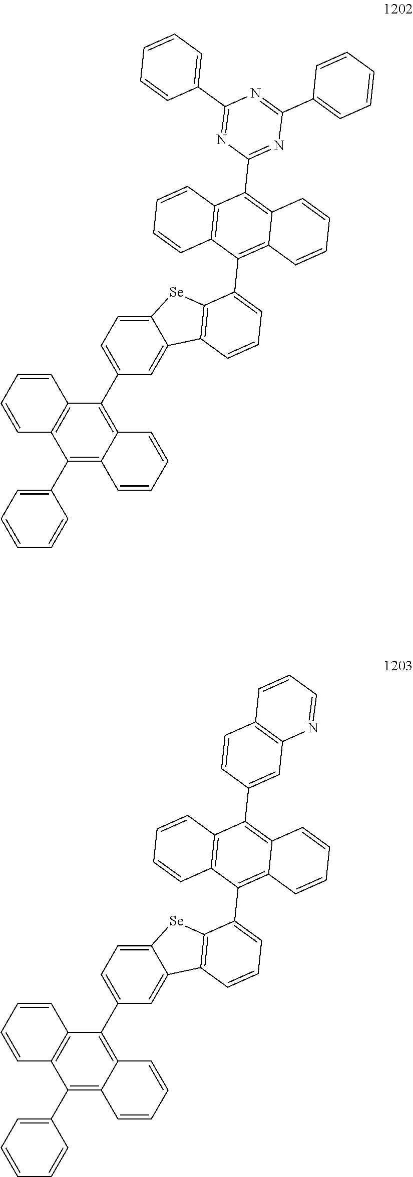 [SMILES: c1ccc(-c2nc(-c3ccccc3)nc(-c3c4ccccc4c(-c4cccc5c4[se]c4ccc(-c6c7ccccc7c(-c7ccccc7)c7ccccc67)cc45)c4ccccc34)n2)cc1 ; c1ccc(-c2c3ccccc3c(-c3ccc4[se]c5c(-c6c7ccccc7c(-c7ccc8cccnc8c7)c7ccccc67)cccc5c4c3)c3ccccc23)cc1]

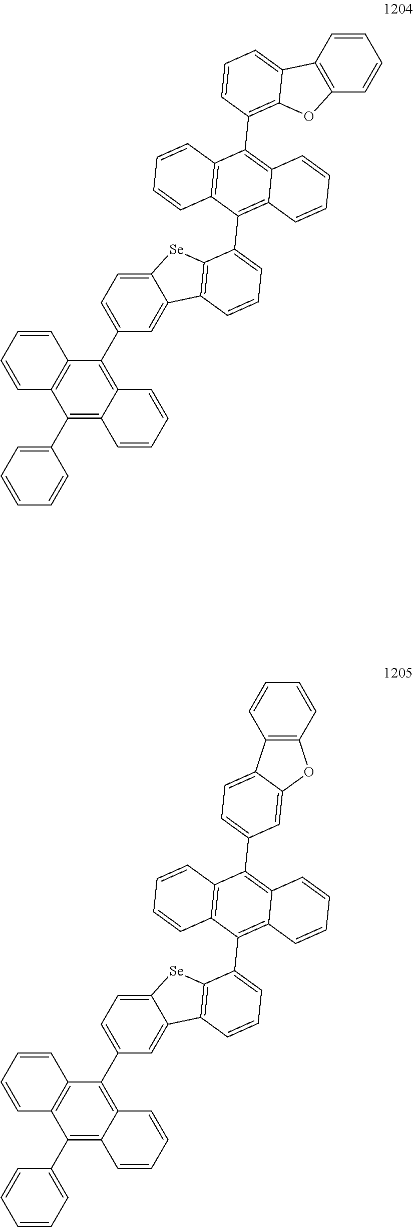 [SMILES: c1ccc(-c2c3ccccc3c(-c3ccc4[se]c5c(-c6c7ccccc7c(-c7cccc8c7oc7ccccc78)c7ccccc67)cccc5c4c3)c3ccccc23)cc1 ; c1ccc(-c2c3ccccc3c(-c3ccc4[se]c5c(-c6c7ccccc7c(-c7ccc8c(c7)oc7ccccc78)c7ccccc67)cccc5c4c3)c3ccccc23)cc1]

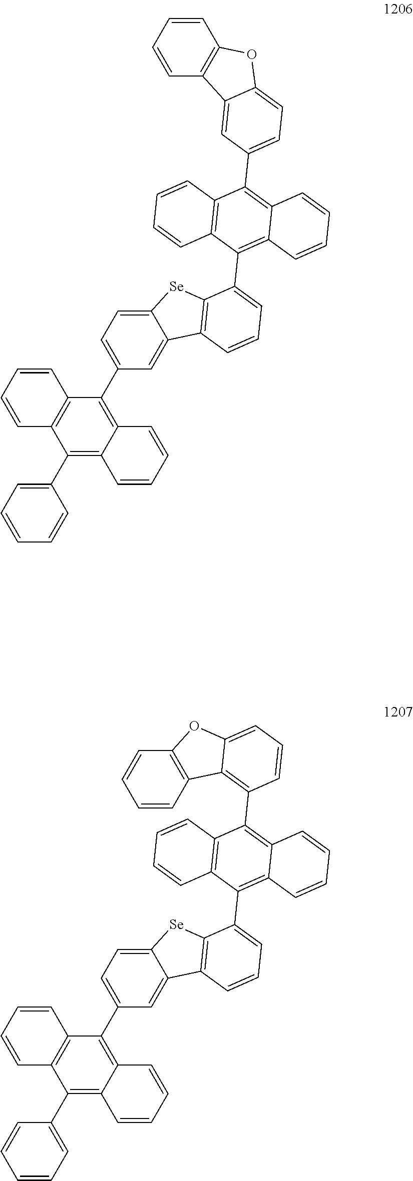 [SMILES: c1ccc(-c2c3ccccc3c(-c3ccc4[se]c5c(-c6c7ccccc7c(-c7ccc8oc9ccccc9c8c7)c7ccccc67)cccc5c4c3)c3ccccc23)cc1 ; c1ccc(-c2c3ccccc3c(-c3ccc4[se]c5c(-c6c7ccccc7c(-c7cccc8oc9ccccc9c78)c7ccccc67)cccc5c4c3)c3ccccc23)cc1]

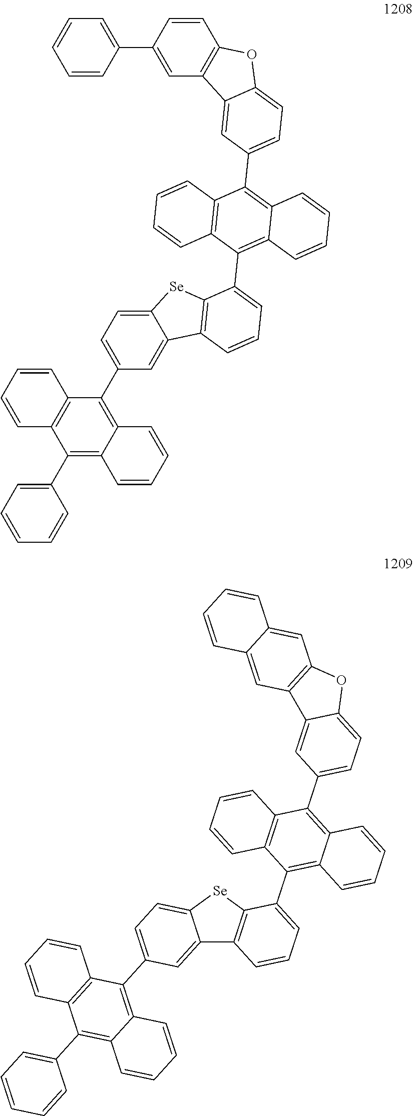 [SMILES: c1ccc(-c2ccc3oc4ccc(-c5c6ccccc6c(-c6cccc7c6[se]c6ccc(-c8c9ccccc9c(-c9ccccc9)c9ccccc89)cc67)c6ccccc56)cc4c3c2)cc1 ; c1ccc(-c2c3ccccc3c(-c3ccc4[se]c5c(-c6c7ccccc7c(-c7ccc8oc9cc%10ccccc%10cc9c8c7)c7ccccc67)cccc5c4c3)c3ccccc23)cc1]

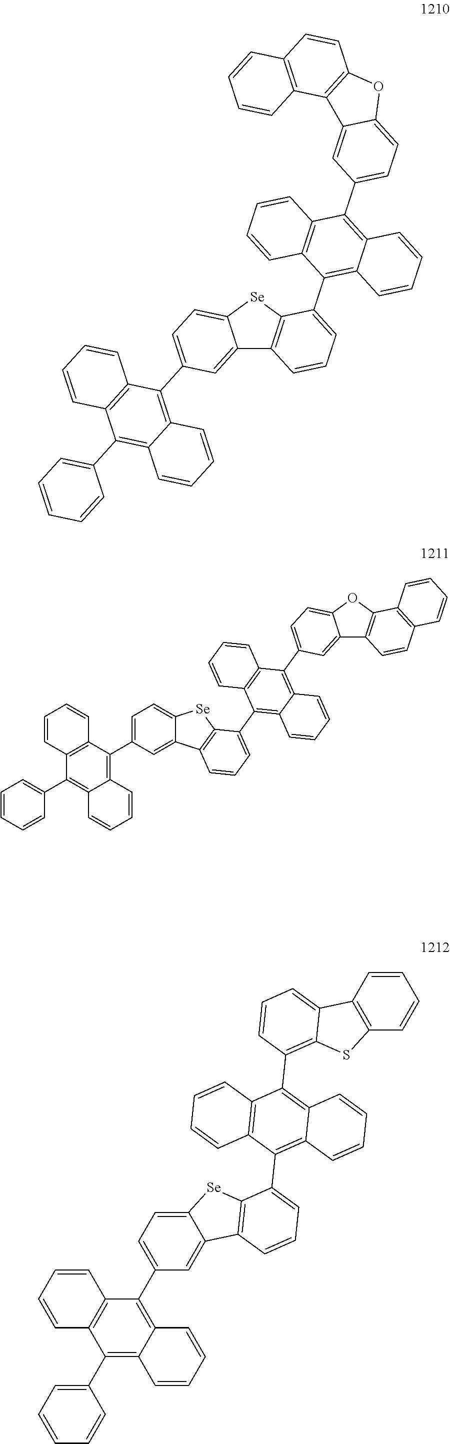 [SMILES: c1ccc(-c2c3ccccc3c(-c3ccc4[se]c5c(-c6c7ccccc7c(-c7ccc8oc9ccc%10ccccc%10c9c8c7)c7ccccc67)cccc5c4c3)c3ccccc23)cc1 ; c1ccc(-c2c3ccccc3c(-c3ccc4[se]c5c(-c6c7ccccc7c(-c7ccc8oc9c%10ccccc%10ccc9c8c7)c7ccccc67)cccc5c4c3)c3ccccc23)cc1 ; c1ccc(-c2c3ccccc3c(-c3ccc4[se]c5c(-c6c7ccccc7c(-c7cccc8c7sc7ccccc78)c7ccccc67)cccc5c4c3)c3ccccc23)cc1]

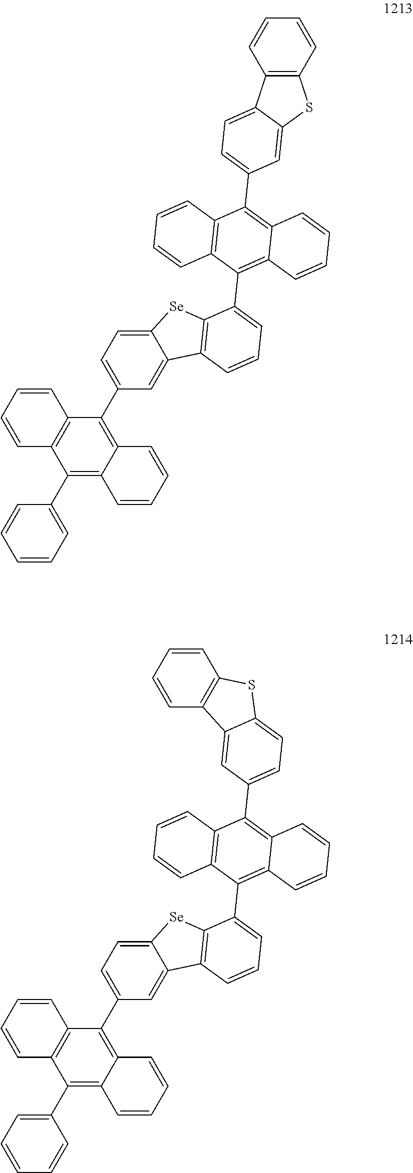 [SMILES: c1ccc(-c2c3ccccc3c(-c3ccc4[se]c5c(-c6c7ccccc7c(-c7ccc8c(c7)sc7ccccc78)c7ccccc67)cccc5c4c3)c3ccccc23)cc1 ; c1ccc(-c2c3ccccc3c(-c3ccc4[se]c5c(-c6c7ccccc7c(-c7ccc8sc9ccccc9c8c7)c7ccccc67)cccc5c4c3)c3ccccc23)cc1]

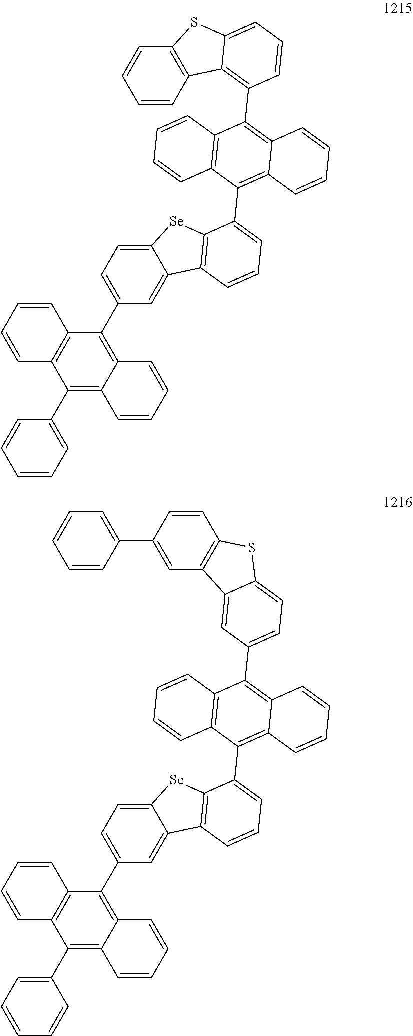

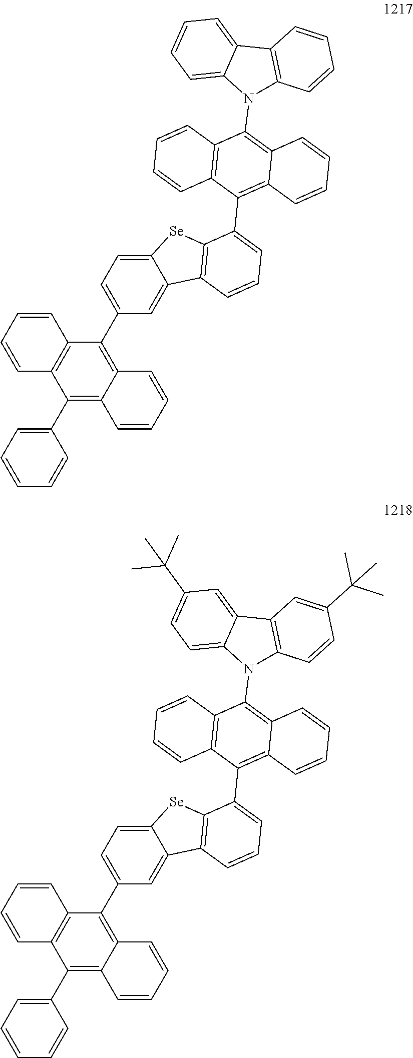 [SMILES: c1ccc(-c2c3ccccc3c(-c3ccc4[se]c5c(-c6c7ccccc7c(-n7c8ccccc8c8ccccc87)c7ccccc67)cccc5c4c3)c3ccccc23)cc1 ; CC(C)(C)c1ccc2c(c1)c1cc(C(C)(C)C)ccc1n2-c1c2ccccc2c(-c2cccc3c2[se]c2ccc(-c4c5ccccc5c(-c5ccccc5)c5ccccc45)cc23)c2ccccc12]

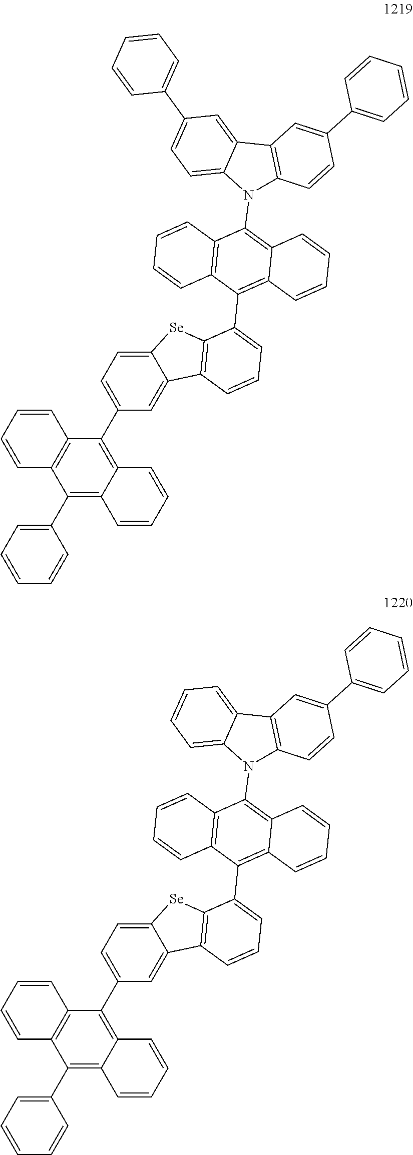 [SMILES: c1ccc(-c2ccc3c(c2)c2cc(-c4ccccc4)ccc2n3-c2c3ccccc3c(-c3cccc4c3[se]c3ccc(-c5c6ccccc6c(-c6ccccc6)c6ccccc56)cc34)c3ccccc23)cc1 ; c1ccc(-c2ccc3c(c2)c2ccccc2n3-c2c3ccccc3c(-c3cccc4c3[se]c3ccc(-c5c6ccccc6c(-c6ccccc6)c6ccccc56)cc34)c3ccccc23)cc1]

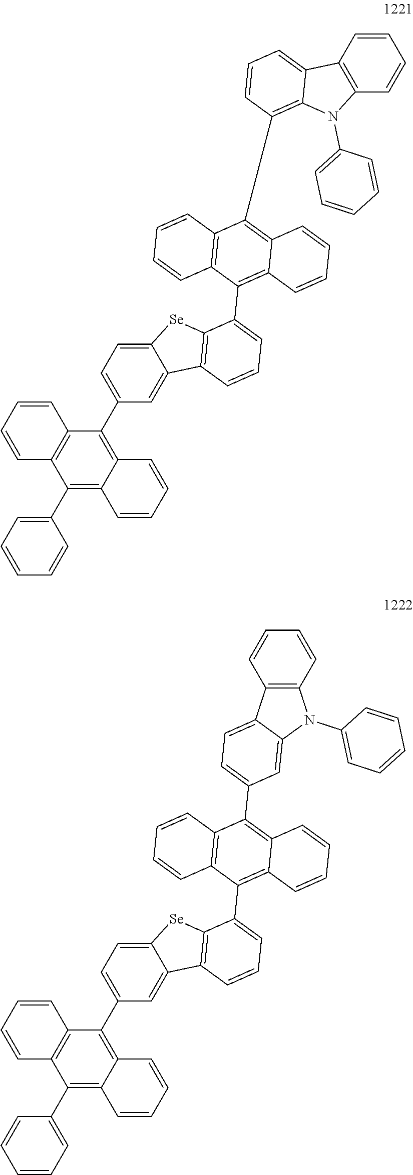 [SMILES: c1ccc(-c2c3ccccc3c(-c3ccc4[se]c5c(-c6c7ccccc7c(-c7cccc8c9ccccc9n(-c9ccccc9)c78)c7ccccc67)cccc5c4c3)c3ccccc23)cc1 ; c1ccc(-c2c3ccccc3c(-c3ccc4[se]c5c(-c6c7ccccc7c(-c7ccc8c9ccccc9n(-c9ccccc9)c8c7)c7ccccc67)cccc5c4c3)c3ccccc23)cc1]

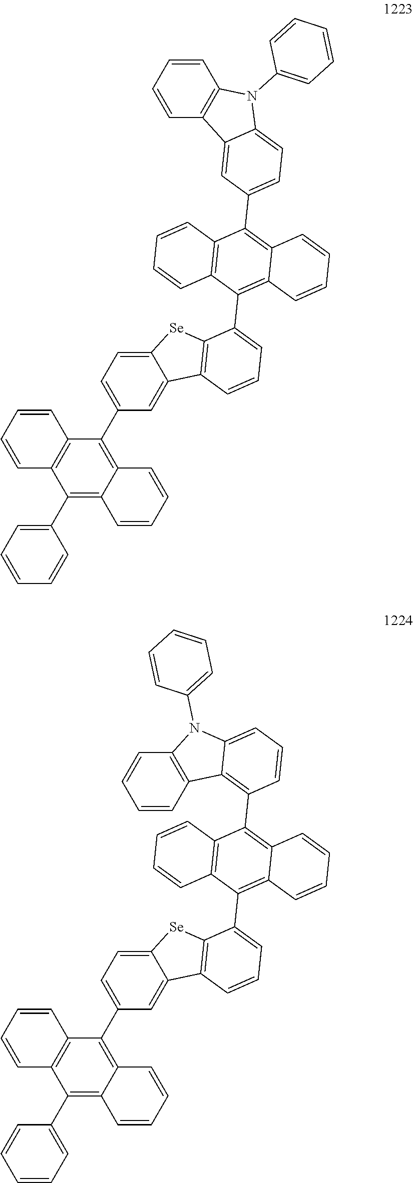 [SMILES: c1ccc(-c2c3ccccc3c(-c3ccc4[se]c5c(-c6c7ccccc7c(-c7ccc8c(c7)c7ccccc7n8-c7ccccc7)c7ccccc67)cccc5c4c3)c3ccccc23)cc1 ; c1ccc(-c2c3ccccc3c(-c3ccc4[se]c5c(-c6c7ccccc7c(-c7cccc8c7c7ccccc7n8-c7ccccc7)c7ccccc67)cccc5c4c3)c3ccccc23)cc1]

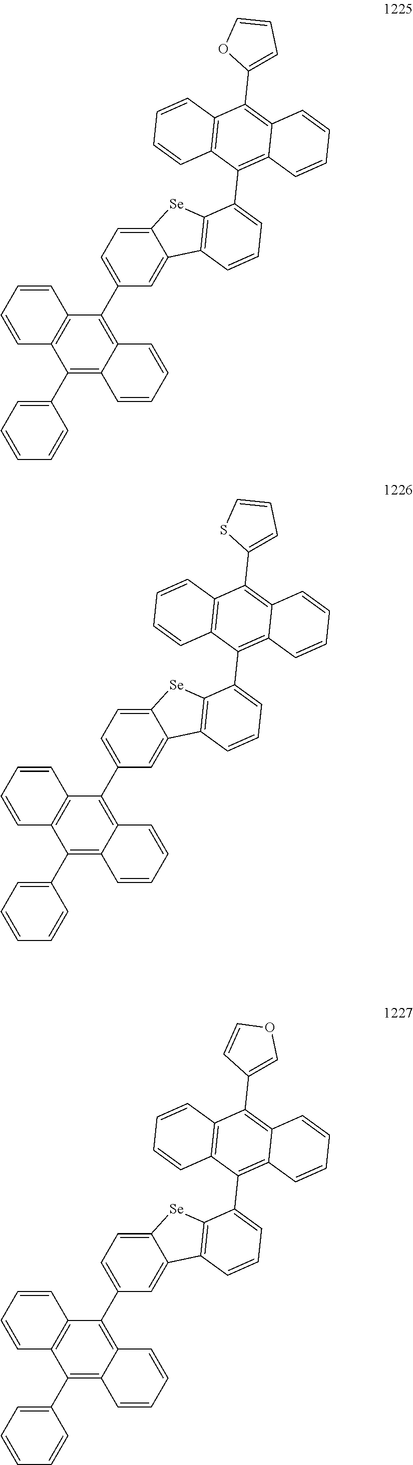 [SMILES: c1ccc(-c2c3ccccc3c(-c3ccc4[se]c5c(-c6c7ccccc7c(-c7ccco7)c7ccccc67)cccc5c4c3)c3ccccc23)cc1 ; c1ccc(-c2c3ccccc3c(-c3ccc4[se]c5c(-c6c7ccccc7c(-c7cccs7)c7ccccc67)cccc5c4c3)c3ccccc23)cc1 ; c1ccc(-c2c3ccccc3c(-c3ccc4[se]c5c(-c6c7ccccc7c(-c7ccoc7)c7ccccc67)cccc5c4c3)c3ccccc23)cc1]

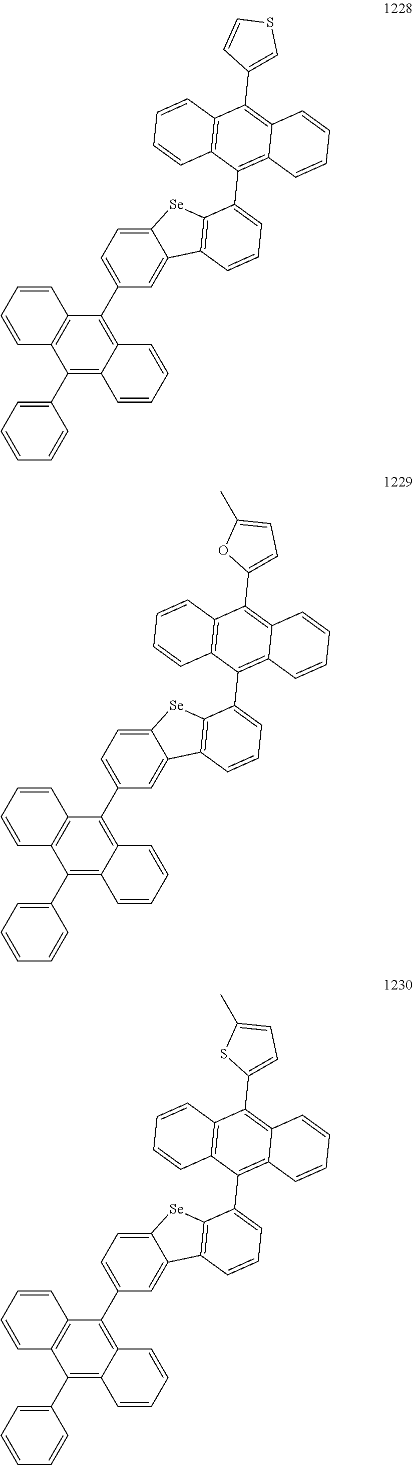 [SMILES: c1ccc(-c2c3ccccc3c(-c3ccc4[se]c5c(-c6c7ccccc7c(-c7ccsc7)c7ccccc67)cccc5c4c3)c3ccccc23)cc1 ; Cc1ccc(-c2c3ccccc3c(-c3cccc4c3[se]c3ccc(-c5c6ccccc6c(-c6ccccc6)c6ccccc56)cc34)c3ccccc23)o1 ; Cc1ccc(-c2c3ccccc3c(-c3cccc4c3[se]c3ccc(-c5c6ccccc6c(-c6ccccc6)c6ccccc56)cc34)c3ccccc23)s1]

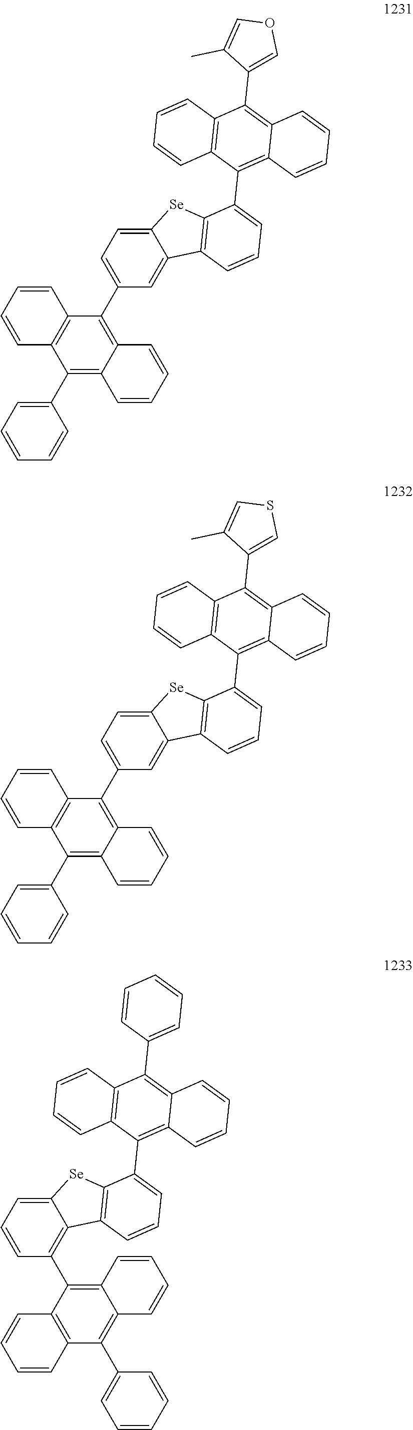 [SMILES: Cc1cocc1-c1c2ccccc2c(-c2cccc3c2[se]c2ccc(-c4c5ccccc5c(-c5ccccc5)c5ccccc45)cc23)c2ccccc12 ; Cc1cscc1-c1c2ccccc2c(-c2cccc3c2[se]c2ccc(-c4c5ccccc5c(-c5ccccc5)c5ccccc45)cc23)c2ccccc12 ; c1ccc(-c2c3ccccc3c(-c3cccc4c3[se]c3cccc(-c5c6ccccc6c(-c6ccccc6)c6ccccc56)c34)c3ccccc23)cc1]

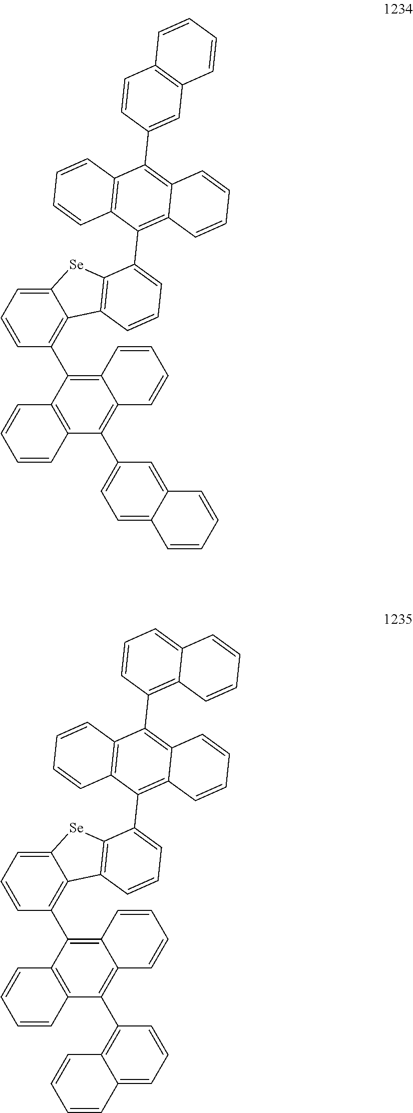 [SMILES: c1ccc2cc(-c3c4ccccc4c(-c4cccc5c4[se]c4cccc(-c6c7ccccc7c(-c7ccc8ccccc8c7)c7ccccc67)c45)c4ccccc34)ccc2c1 ; c1ccc2c(-c3c4ccccc4c(-c4cccc5c4[se]c4cccc(-c6c7ccccc7c(-c7cccc8ccccc78)c7ccccc67)c45)c4ccccc34)cccc2c1]

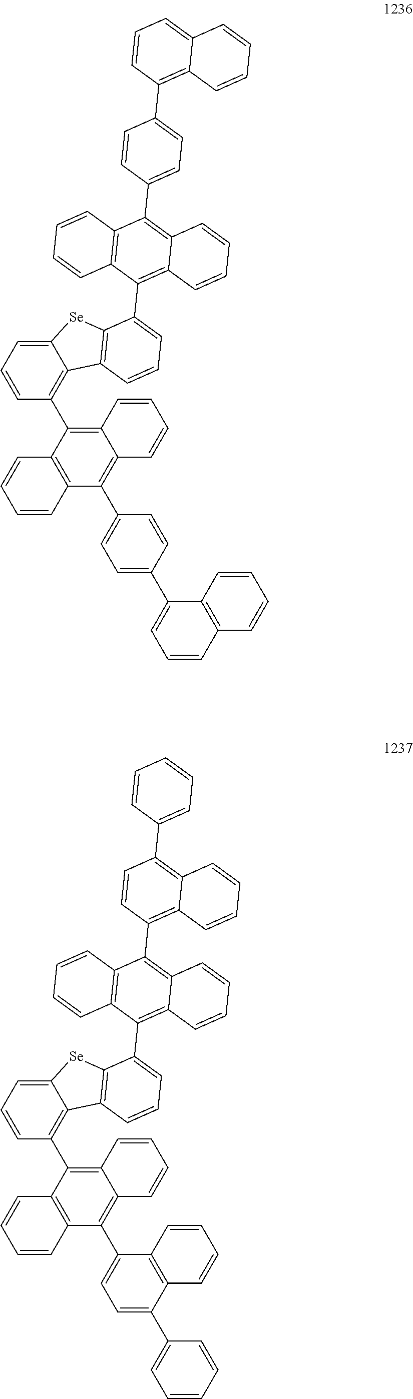 [SMILES: c1ccc2c(-c3ccc(-c4c5ccccc5c(-c5cccc6c5[se]c5cccc(-c7c8ccccc8c(-c8ccc(-c9cccc%10ccccc9%10)cc8)c8ccccc78)c56)c5ccccc45)cc3)cccc2c1 ; c1ccc(-c2ccc(-c3c4ccccc4c(-c4cccc5c4[se]c4cccc(-c6c7ccccc7c(-c7ccc(-c8ccccc8)c8ccccc78)c7ccccc67)c45)c4ccccc34)c3ccccc23)cc1]

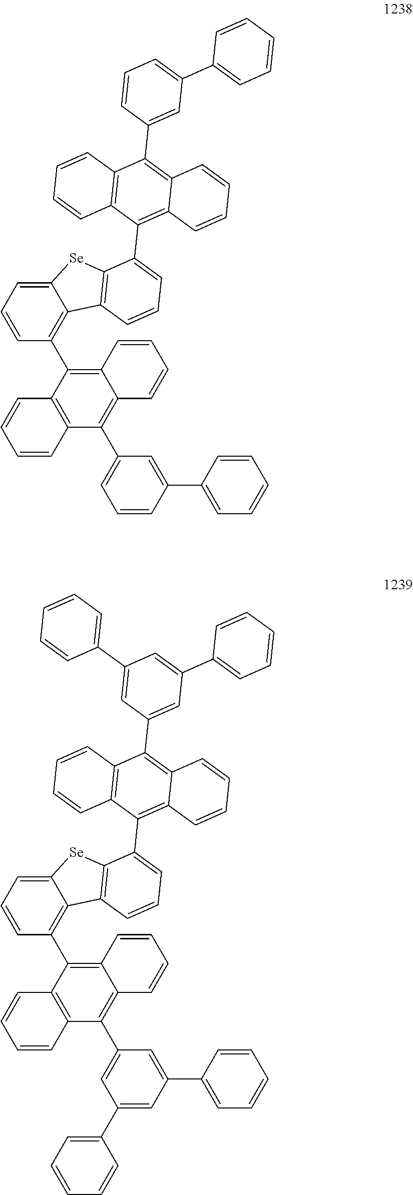 [SMILES: c1ccc(-c2cccc(-c3c4ccccc4c(-c4cccc5c4[se]c4cccc(-c6c7ccccc7c(-c7cccc(-c8ccccc8)c7)c7ccccc67)c45)c4ccccc34)c2)cc1 ; c1ccc(-c2cc(-c3ccccc3)cc(-c3c4ccccc4c(-c4cccc5c4[se]c4cccc(-c6c7ccccc7c(-c7cc(-c8ccccc8)cc(-c8ccccc8)c7)c7ccccc67)c45)c4ccccc34)c2)cc1]

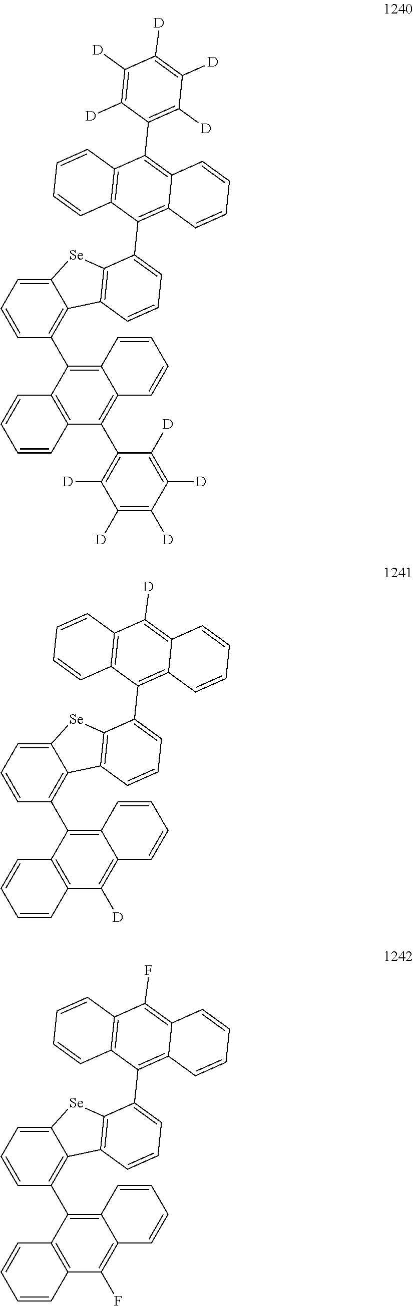 [SMILES: [2H]c1c([2H])c([2H])c(-c2c3ccccc3c(-c3cccc4c3[se]c3cccc(-c5c6ccccc6c(-c6c([2H])c([2H])c([2H])c([2H])c6[2H])c6ccccc56)c34)c3ccccc23)c([2H])c1[2H] ; [2H]c1c2ccccc2c(-c2cccc3c2[se]c2cccc(-c4c5ccccc5c([2H])c5ccccc45)c23)c2ccccc12 ; Fc1c2ccccc2c(-c2cccc3c2[se]c2cccc(-c4c5ccccc5c(F)c5ccccc45)c23)c2ccccc12]

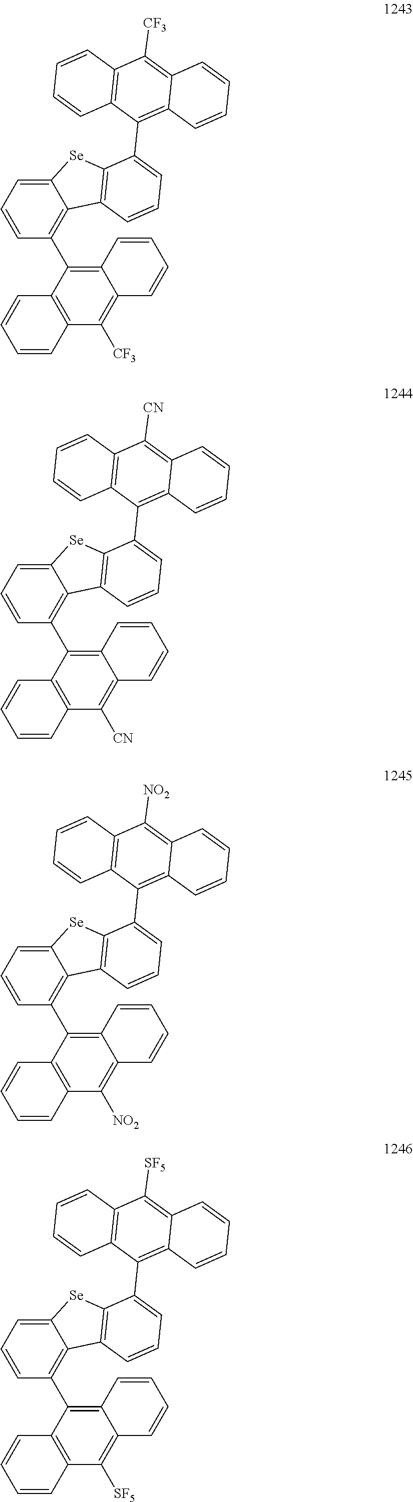

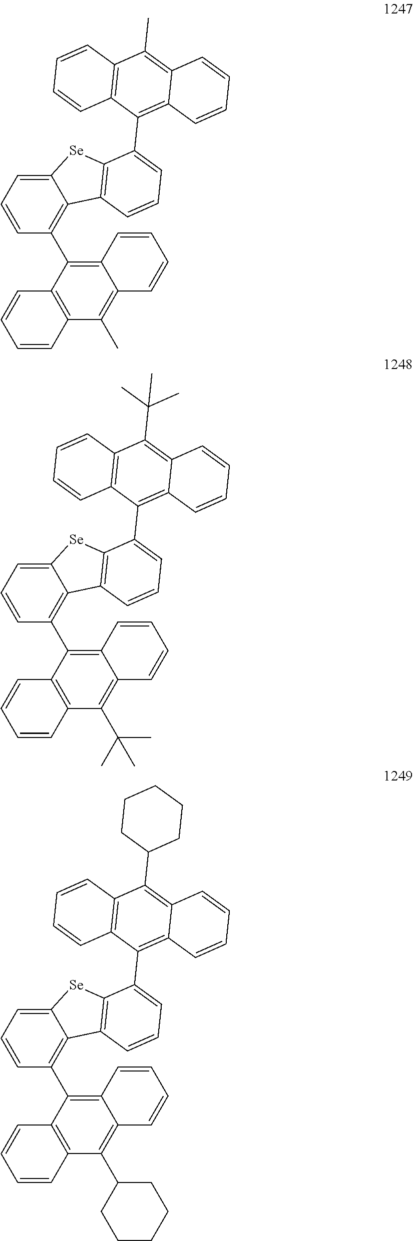

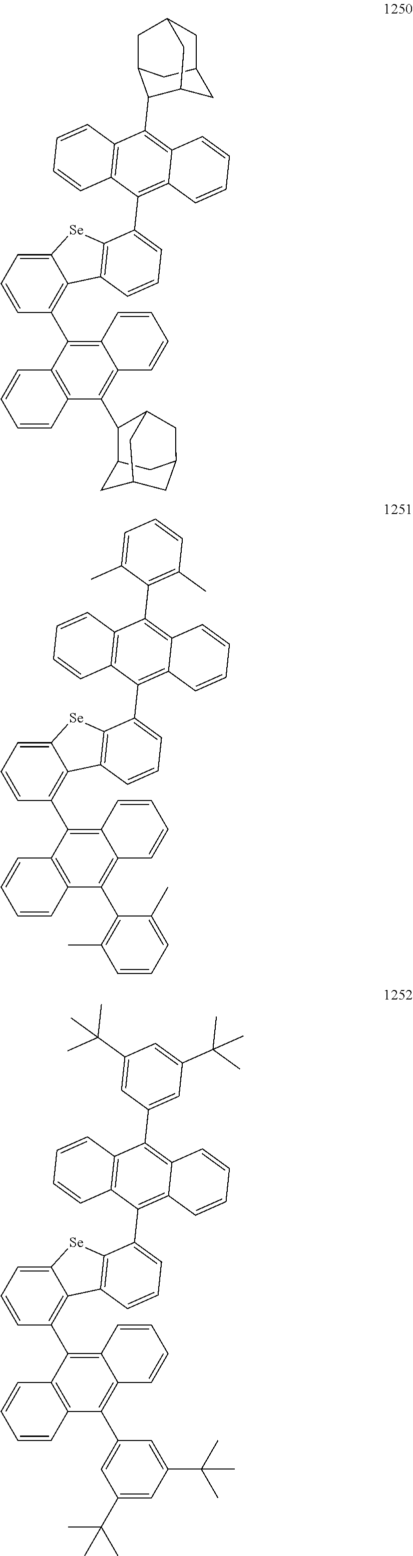

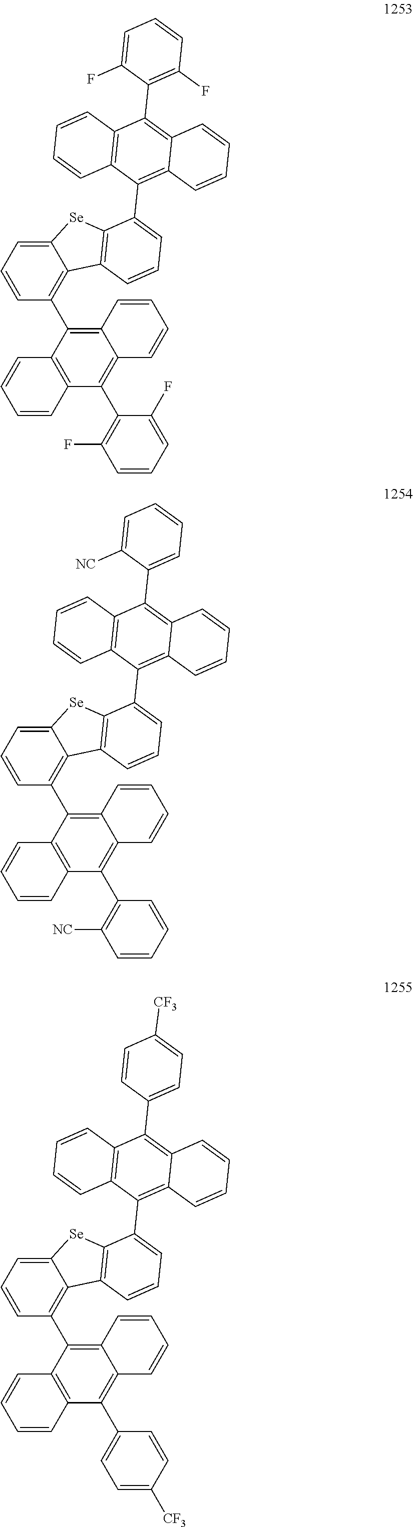

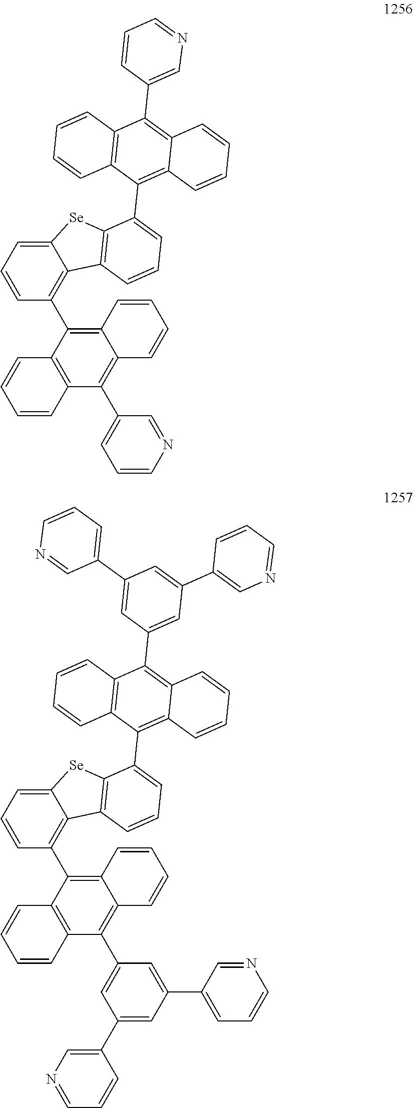 [SMILES: c1cncc(-c2c3ccccc3c(-c3cccc4c3[se]c3cccc(-c5c6ccccc6c(-c6cccnc6)c6ccccc56)c34)c3ccccc23)c1 ; c1cncc(-c2cc(-c3cccnc3)cc(-c3c4ccccc4c(-c4cccc5c4[se]c4cccc(-c6c7ccccc7c(-c7cc(-c8cccnc8)cc(-c8cccnc8)c7)c7ccccc67)c45)c4ccccc34)c2)c1]

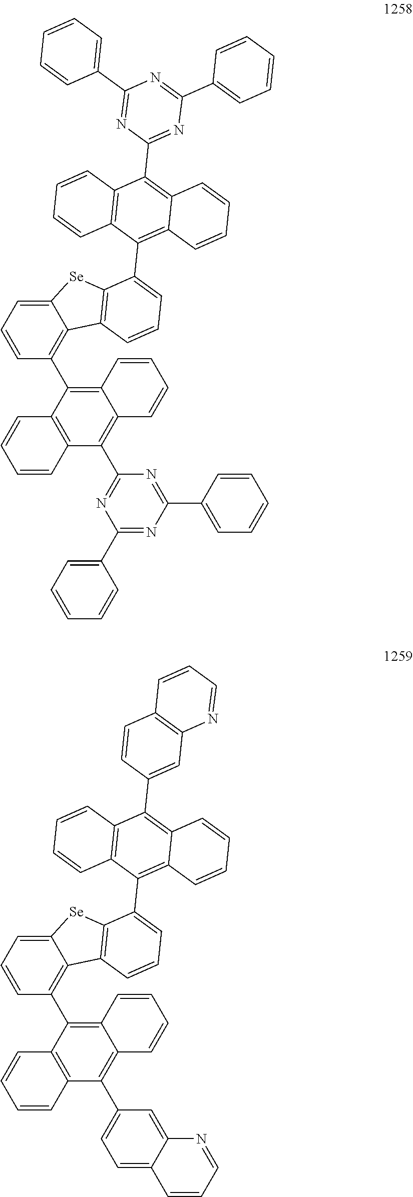 [SMILES: c1ccc(-c2nc(-c3ccccc3)nc(-c3c4ccccc4c(-c4cccc5c4[se]c4cccc(-c6c7ccccc7c(-c7nc(-c8ccccc8)nc(-c8ccccc8)n7)c7ccccc67)c45)c4ccccc34)n2)cc1 ; c1cnc2cc(-c3c4ccccc4c(-c4cccc5c4[se]c4cccc(-c6c7ccccc7c(-c7ccc8cccnc8c7)c7ccccc67)c45)c4ccccc34)ccc2c1]

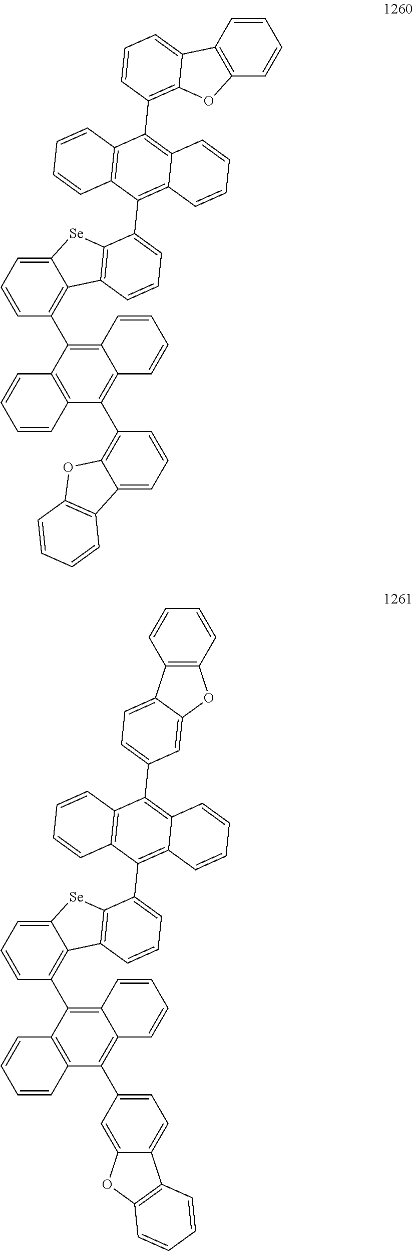 [SMILES: c1ccc2c(c1)oc1c(-c3c4ccccc4c(-c4cccc5c4[se]c4cccc(-c6c7ccccc7c(-c7cccc8c7oc7ccccc78)c7ccccc67)c45)c4ccccc34)cccc12 ; c1ccc2c(c1)oc1cc(-c3c4ccccc4c(-c4cccc5c4[se]c4cccc(-c6c7ccccc7c(-c7ccc8c(c7)oc7ccccc78)c7ccccc67)c45)c4ccccc34)ccc12]

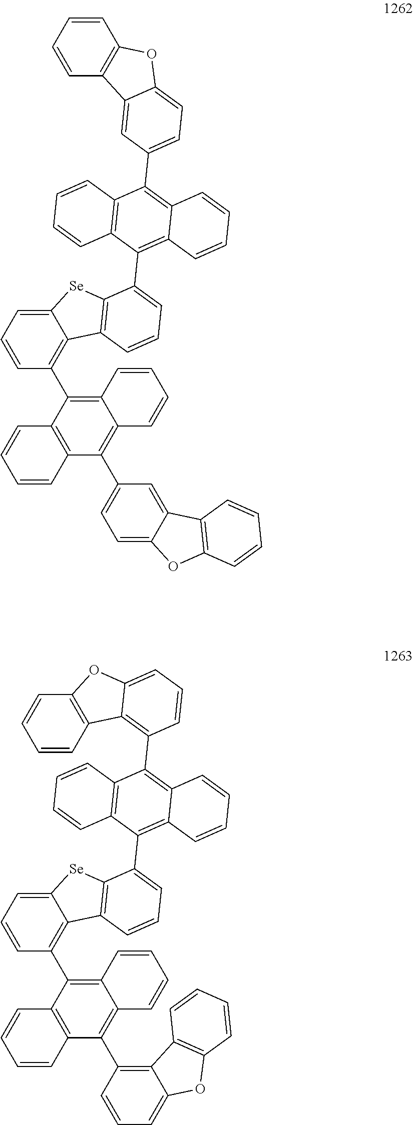 [SMILES: c1ccc2c(c1)oc1ccc(-c3c4ccccc4c(-c4cccc5c4[se]c4cccc(-c6c7ccccc7c(-c7ccc8oc9ccccc9c8c7)c7ccccc67)c45)c4ccccc34)cc12 ; c1ccc2c(c1)oc1cccc(-c3c4ccccc4c(-c4cccc5c4[se]c4cccc(-c6c7ccccc7c(-c7cccc8oc9ccccc9c78)c7ccccc67)c45)c4ccccc34)c12]

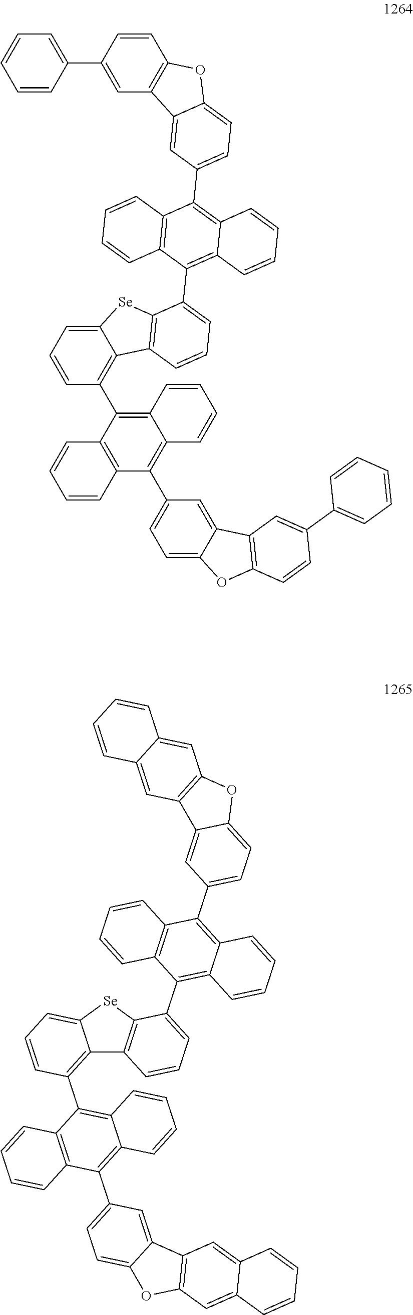 [SMILES: c1ccc(-c2ccc3oc4ccc(-c5c6ccccc6c(-c6cccc7c6[se]c6cccc(-c8c9ccccc9c(-c9ccc%10oc%11ccc(-c%12ccccc%12)cc%11c%10c9)c9ccccc89)c67)c6ccccc56)cc4c3c2)cc1 ; c1ccc2cc3c(cc2c1)oc1ccc(-c2c4ccccc4c(-c4cccc5c4[se]c4cccc(-c6c7ccccc7c(-c7ccc8oc9cc%10ccccc%10cc9c8c7)c7ccccc67)c45)c4ccccc24)cc13]

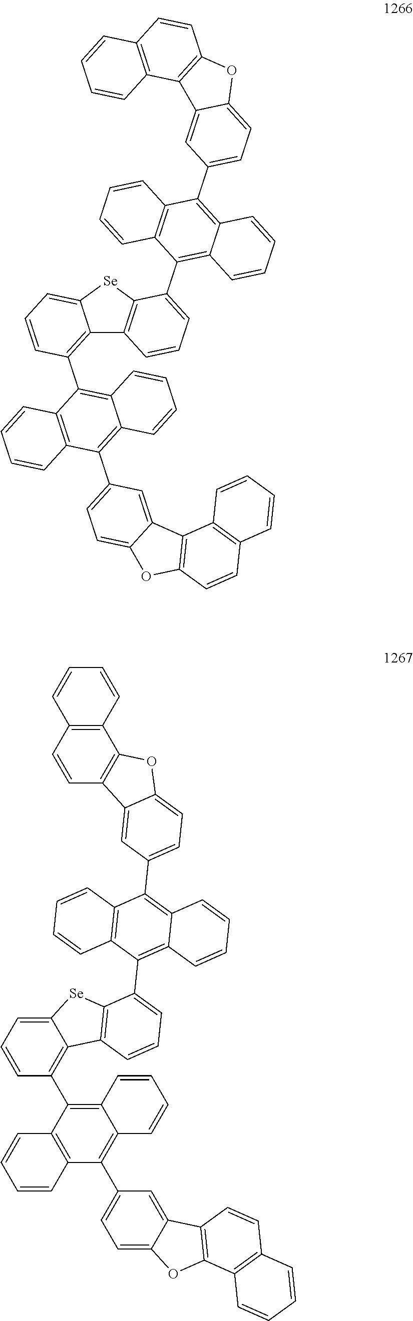 [SMILES: c1ccc2c(c1)ccc1oc3ccc(-c4c5ccccc5c(-c5cccc6c5[se]c5cccc(-c7c8ccccc8c(-c8ccc9oc%10ccc%11ccccc%11c%10c9c8)c8ccccc78)c56)c5ccccc45)cc3c12 ; c1ccc2c(c1)ccc1c3cc(-c4c5ccccc5c(-c5cccc6c5[se]c5cccc(-c7c8ccccc8c(-c8ccc9oc%10c%11ccccc%11ccc%10c9c8)c8ccccc78)c56)c5ccccc45)ccc3oc21]

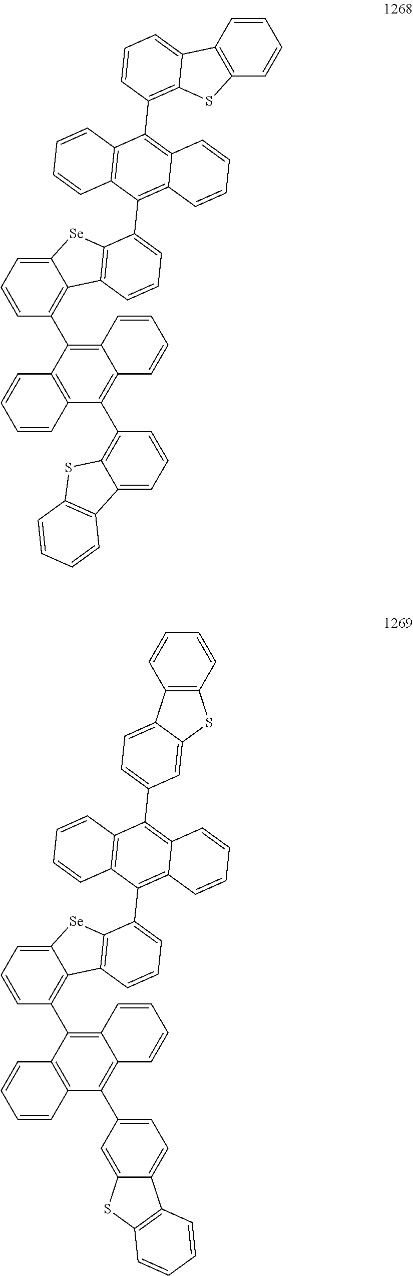 [SMILES: c1ccc2c(c1)sc1c(-c3c4ccccc4c(-c4cccc5c4[se]c4cccc(-c6c7ccccc7c(-c7cccc8c7sc7ccccc78)c7ccccc67)c45)c4ccccc34)cccc12 ; c1ccc2c(c1)sc1cc(-c3c4ccccc4c(-c4cccc5c4[se]c4cccc(-c6c7ccccc7c(-c7ccc8c(c7)sc7ccccc78)c7ccccc67)c45)c4ccccc34)ccc12]

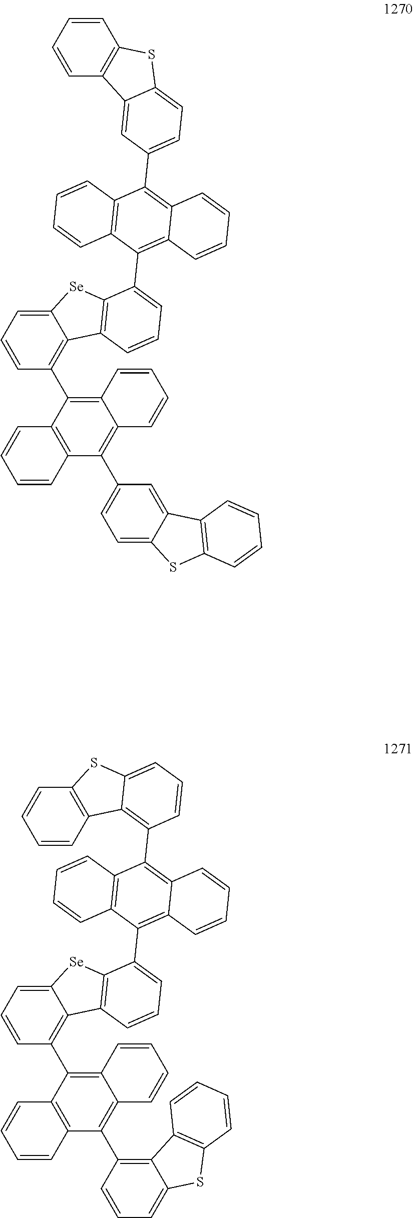 [SMILES: c1ccc2c(c1)sc1ccc(-c3c4ccccc4c(-c4cccc5c4[se]c4cccc(-c6c7ccccc7c(-c7ccc8sc9ccccc9c8c7)c7ccccc67)c45)c4ccccc34)cc12 ; c1ccc2c(c1)sc1cccc(-c3c4ccccc4c(-c4cccc5c4[se]c4cccc(-c6c7ccccc7c(-c7cccc8sc9ccccc9c78)c7ccccc67)c45)c4ccccc34)c12]

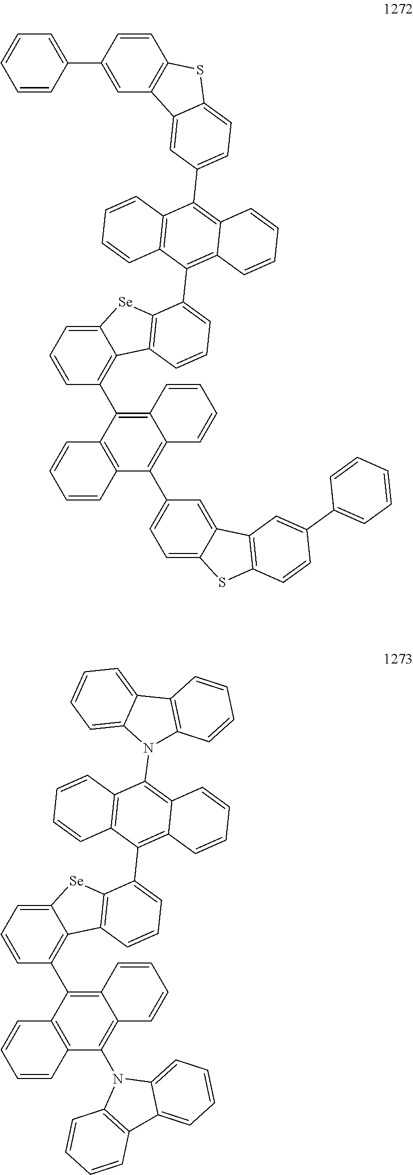 [SMILES: c1ccc(-c2ccc3sc4ccc(-c5c6ccccc6c(-c6cccc7c6[se]c6cccc(-c8c9ccccc9c(-c9ccc%10sc%11ccc(-c%12ccccc%12)cc%11c%10c9)c9ccccc89)c67)c6ccccc56)cc4c3c2)cc1 ; c1cc(-c2c3ccccc3c(-n3c4ccccc4c4ccccc43)c3ccccc23)c2c(c1)[se]c1c(-c3c4ccccc4c(-n4c5ccccc5c5ccccc54)c4ccccc34)cccc12]

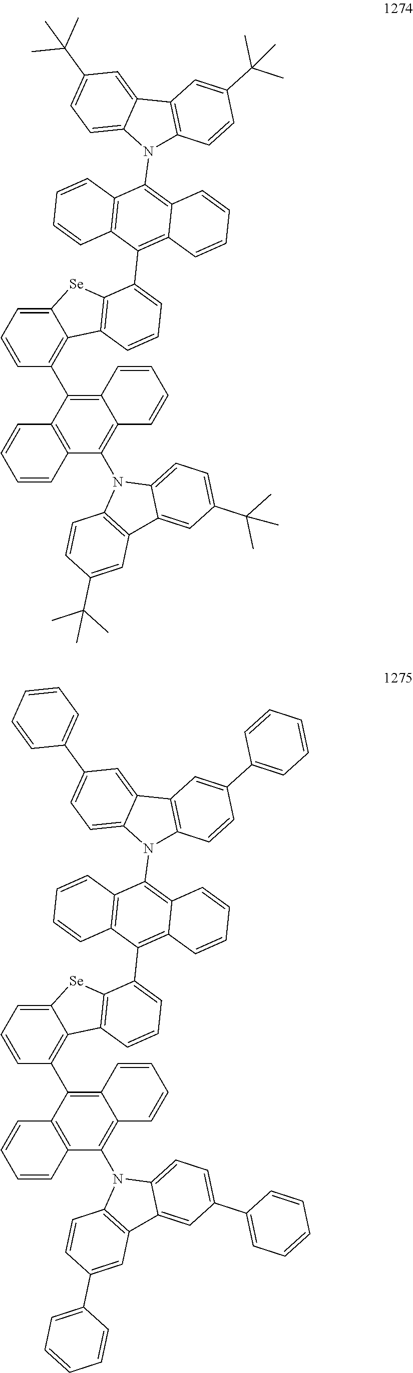 [SMILES: CC(C)(C)c1ccc2c(c1)c1cc(C(C)(C)C)ccc1n2-c1c2ccccc2c(-c2cccc3c2[se]c2cccc(-c4c5ccccc5c(-n5c6ccc(C(C)(C)C)cc6c6cc(C(C)(C)C)ccc65)c5ccccc45)c23)c2ccccc12 ; c1ccc(-c2ccc3c(c2)c2cc(-c4ccccc4)ccc2n3-c2c3ccccc3c(-c3cccc4c3[se]c3cccc(-c5c6ccccc6c(-n6c7ccc(-c8ccccc8)cc7c7cc(-c8ccccc8)ccc76)c6ccccc56)c34)c3ccccc23)cc1]

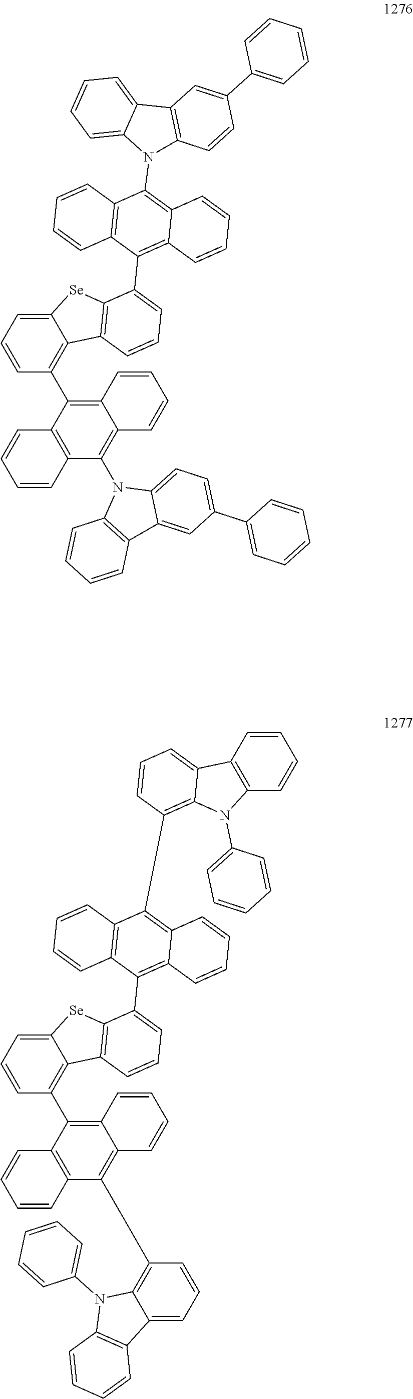 [SMILES: c1ccc(-c2ccc3c(c2)c2ccccc2n3-c2c3ccccc3c(-c3cccc4c3[se]c3cccc(-c5c6ccccc6c(-n6c7ccccc7c7cc(-c8ccccc8)ccc76)c6ccccc56)c34)c3ccccc23)cc1 ; c1ccc(-n2c3ccccc3c3cccc(-c4c5ccccc5c(-c5cccc6c5[se]c5cccc(-c7c8ccccc8c(-c8cccc9c%10ccccc%10n(-c%10ccccc%10)c89)c8ccccc78)c56)c5ccccc45)c32)cc1]

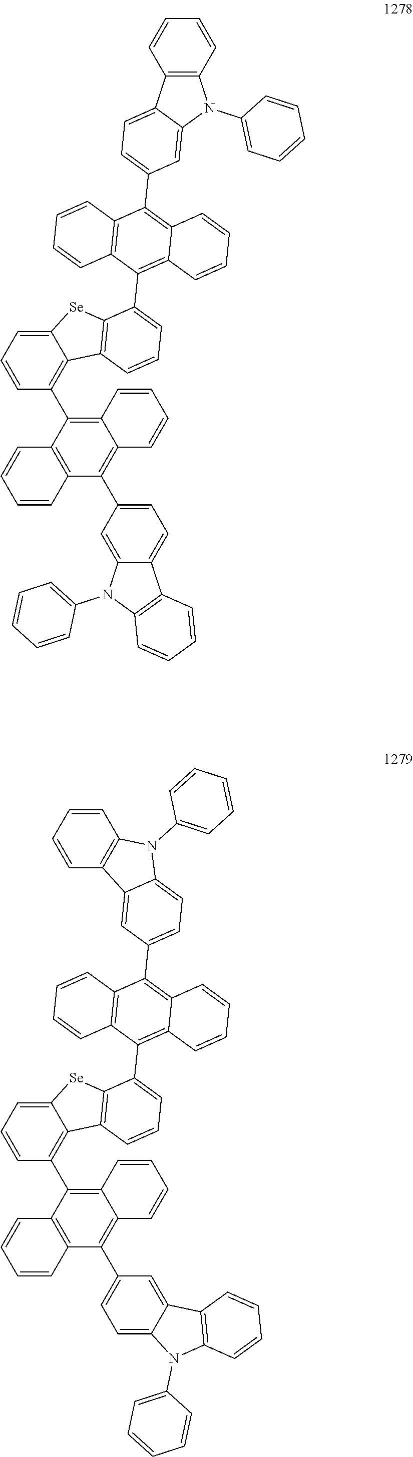 [SMILES: c1ccc(-n2c3ccccc3c3ccc(-c4c5ccccc5c(-c5cccc6c5[se]c5cccc(-c7c8ccccc8c(-c8ccc9c%10ccccc%10n(-c%10ccccc%10)c9c8)c8ccccc78)c56)c5ccccc45)cc32)cc1 ; c1ccc(-n2c3ccccc3c3cc(-c4c5ccccc5c(-c5cccc6c5[se]c5cccc(-c7c8ccccc8c(-c8ccc9c(c8)c8ccccc8n9-c8ccccc8)c8ccccc78)c56)c5ccccc45)ccc32)cc1]

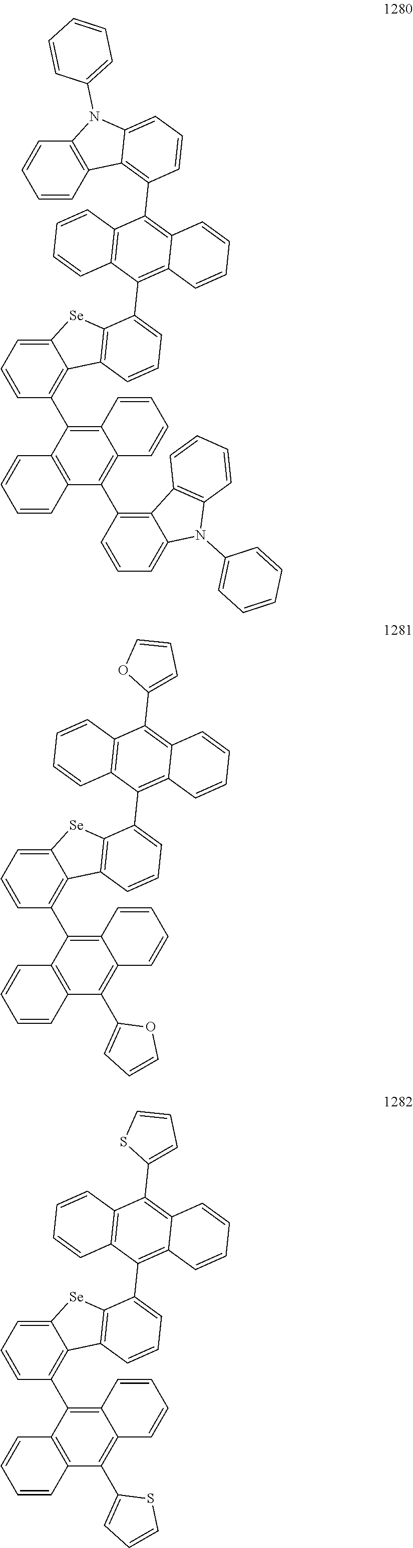

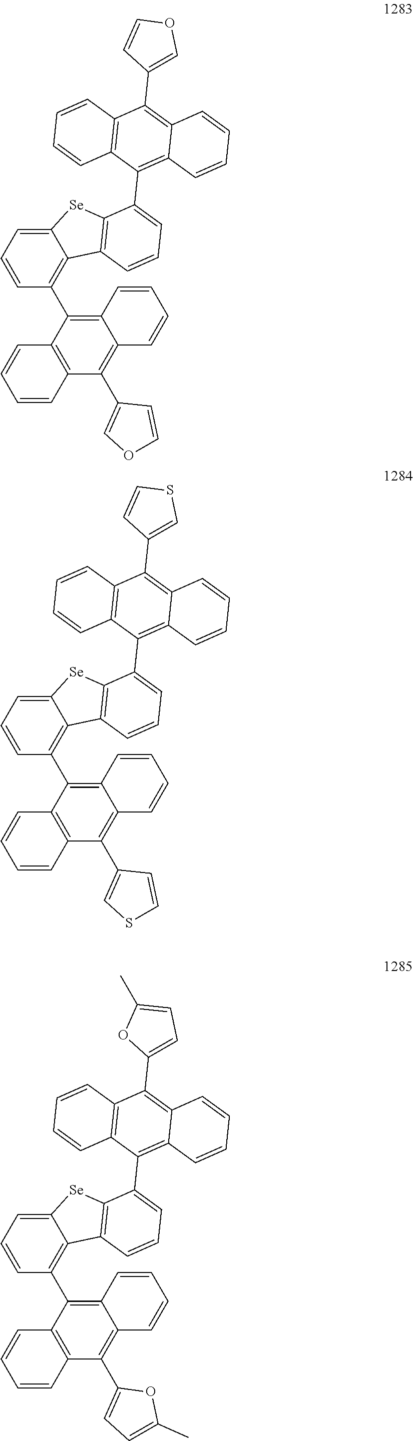

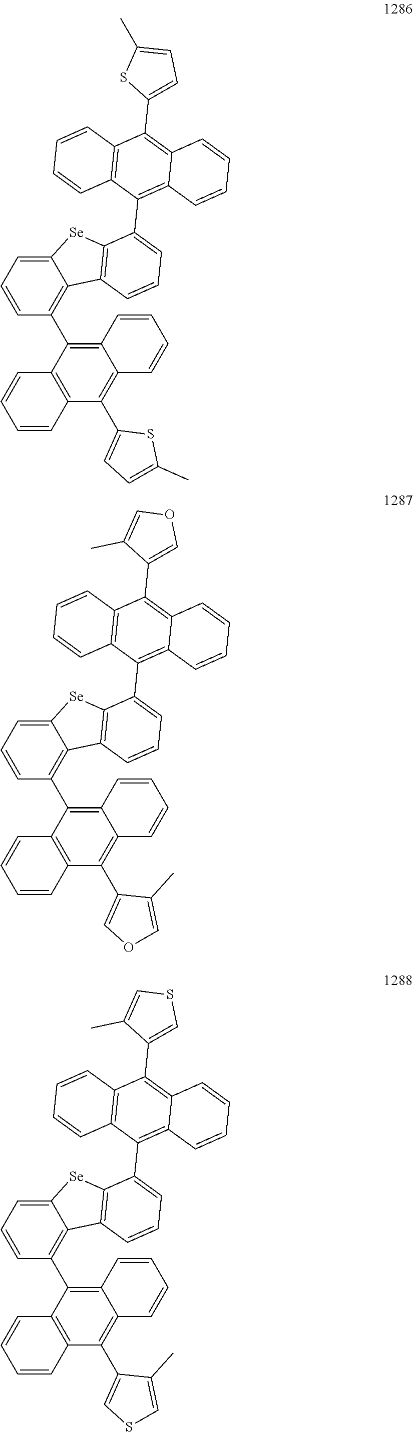

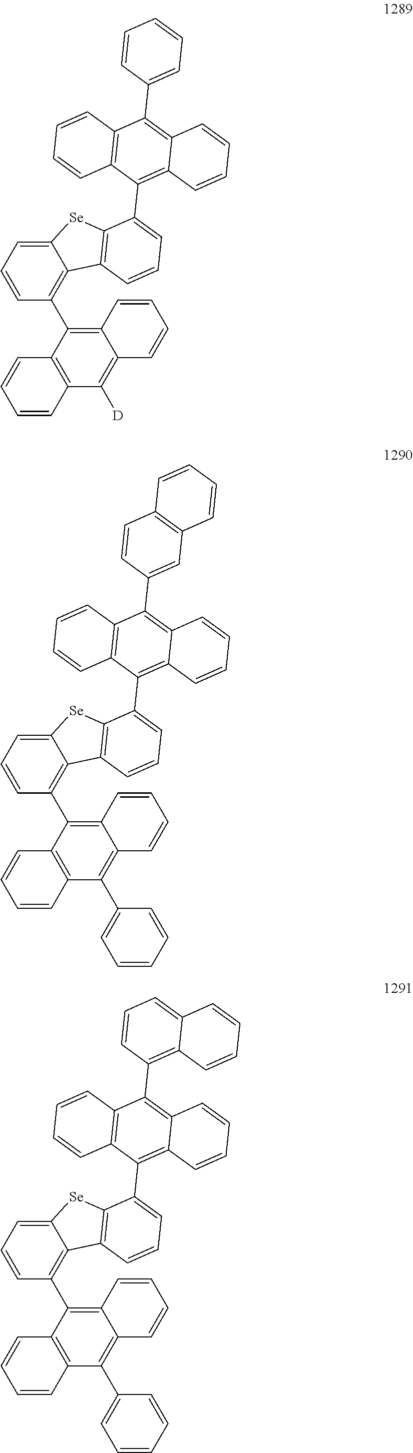

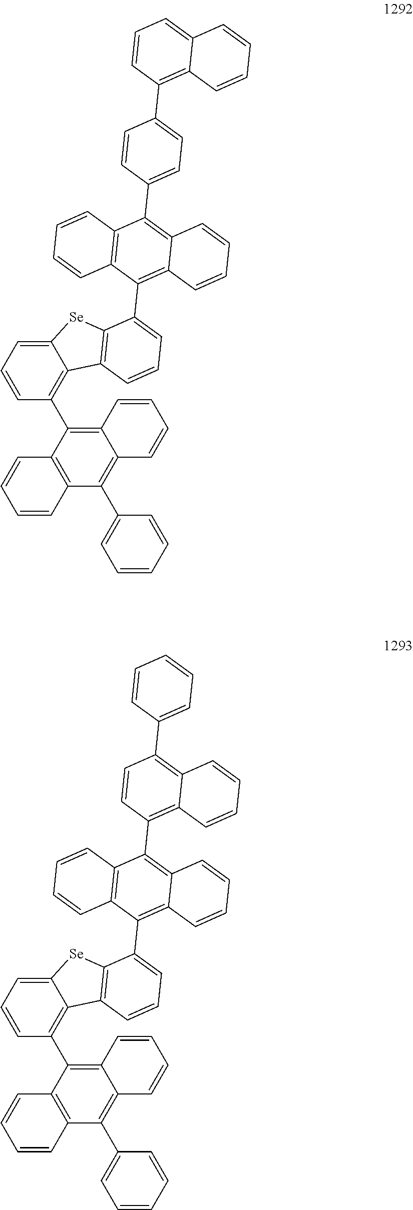 [SMILES: c1ccc(-c2c3ccccc3c(-c3cccc4[se]c5c(-c6c7ccccc7c(-c7ccc(-c8cccc9ccccc89)cc7)c7ccccc67)cccc5c34)c3ccccc23)cc1 ; c1ccc(-c2ccc(-c3c4ccccc4c(-c4cccc5c4[se]c4cccc(-c6c7ccccc7c(-c7ccccc7)c7ccccc67)c45)c4ccccc34)c3ccccc23)cc1]

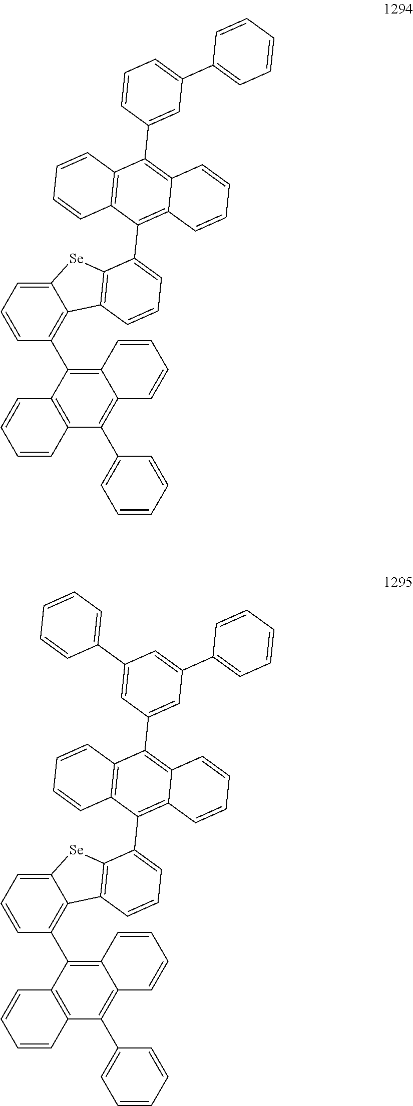 [SMILES: c1ccc(-c2cccc(-c3c4ccccc4c(-c4cccc5c4[se]c4cccc(-c6c7ccccc7c(-c7ccccc7)c7ccccc67)c45)c4ccccc34)c2)cc1 ; c1ccc(-c2cc(-c3ccccc3)cc(-c3c4ccccc4c(-c4cccc5c4[se]c4cccc(-c6c7ccccc7c(-c7ccccc7)c7ccccc67)c45)c4ccccc34)c2)cc1]

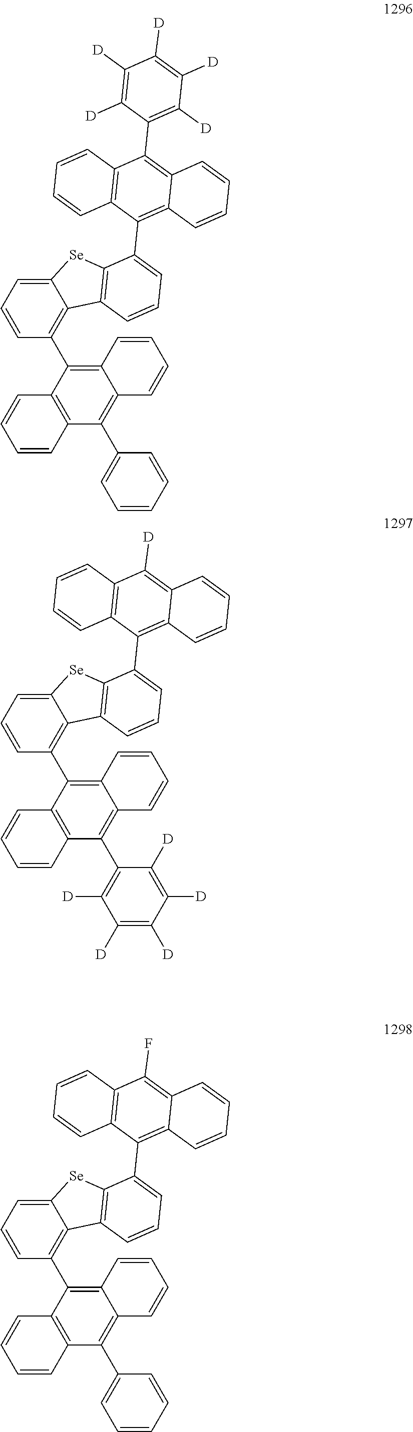

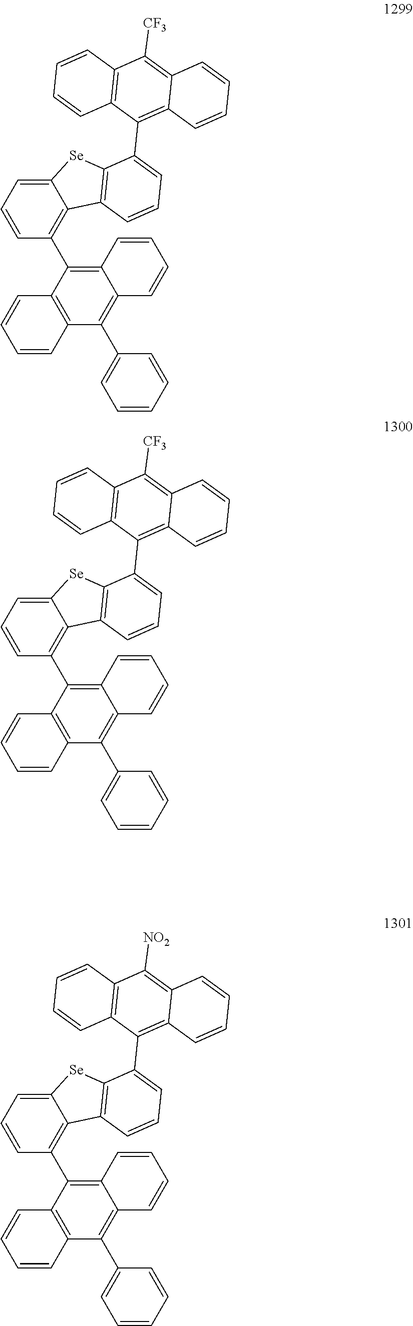

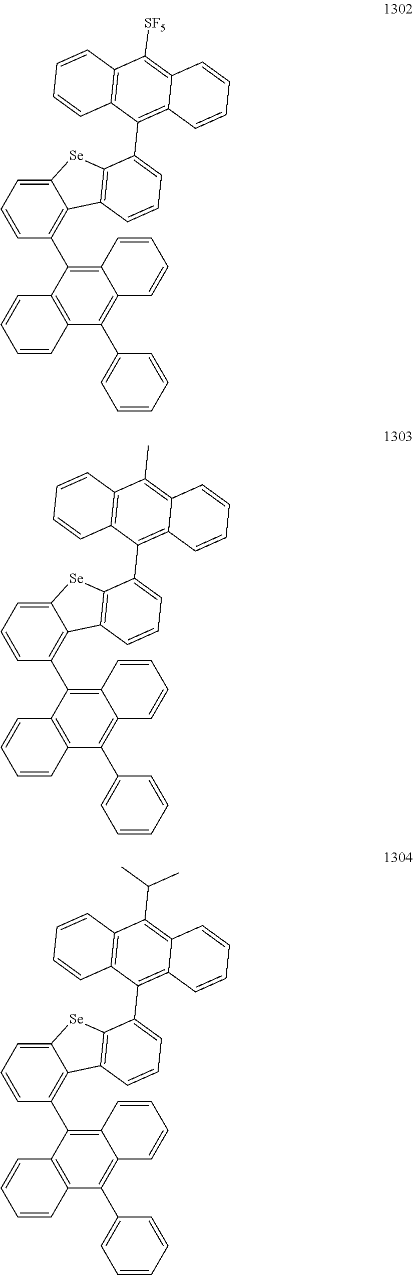

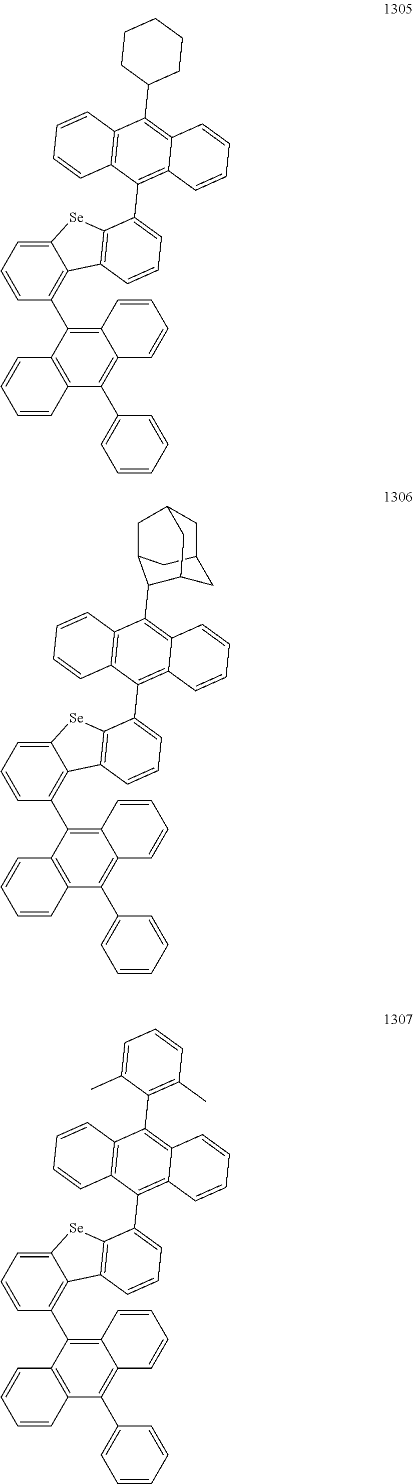

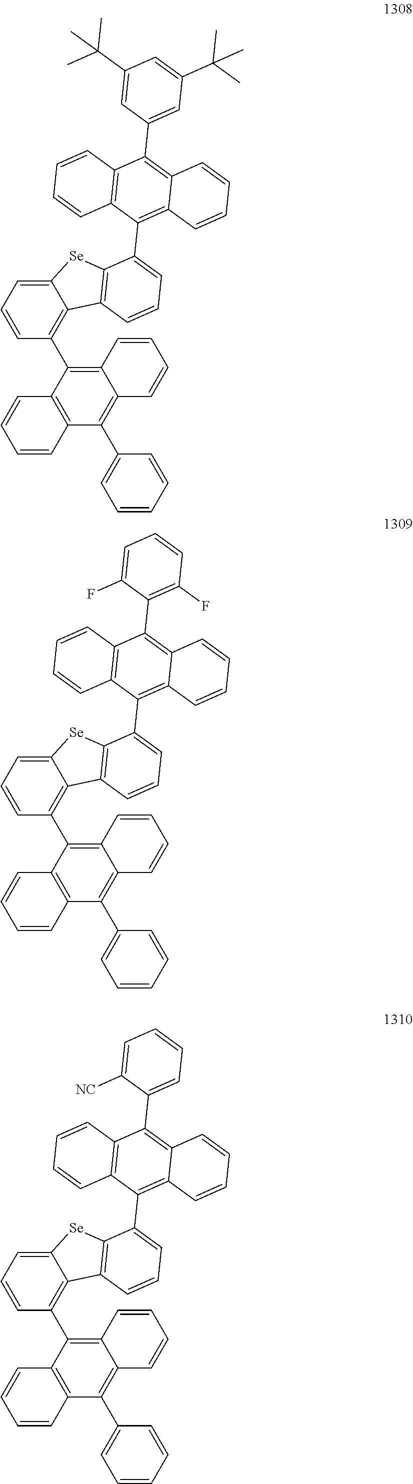

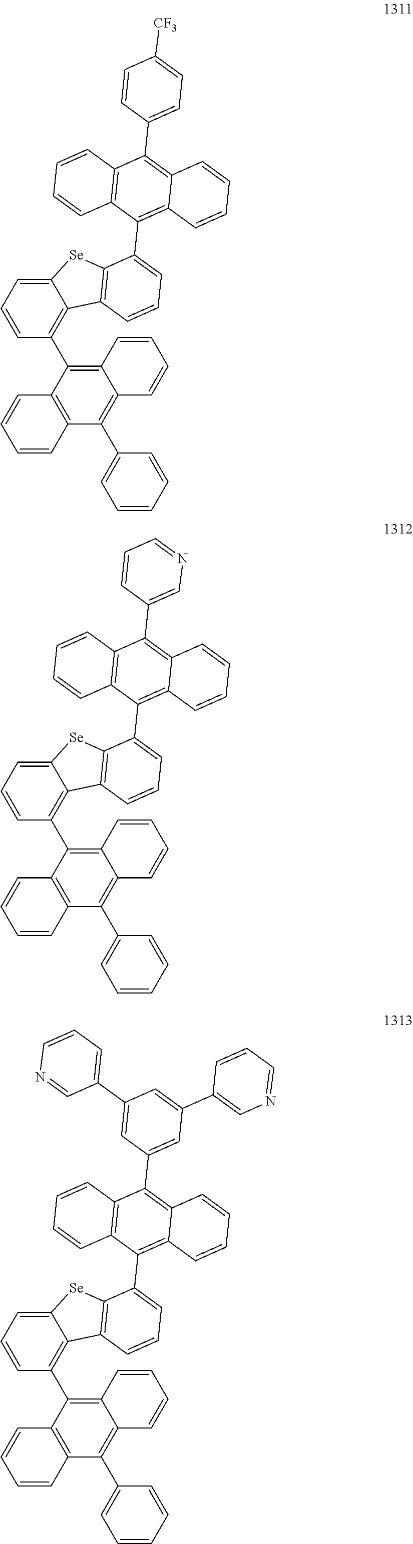

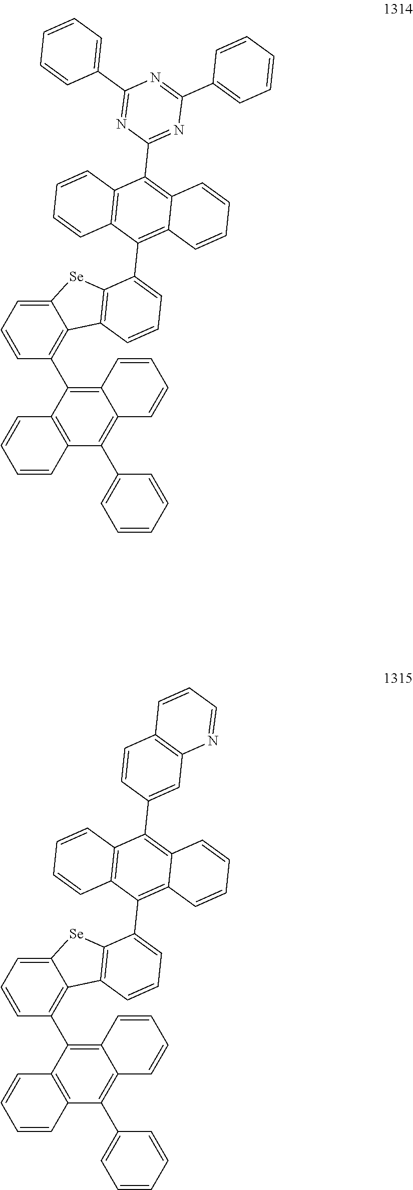 [SMILES: c1ccc(-c2nc(-c3ccccc3)nc(-c3c4ccccc4c(-c4cccc5c4[se]c4cccc(-c6c7ccccc7c(-c7ccccc7)c7ccccc67)c45)c4ccccc34)n2)cc1 ; c1ccc(-c2c3ccccc3c(-c3cccc4[se]c5c(-c6c7ccccc7c(-c7ccc8cccnc8c7)c7ccccc67)cccc5c34)c3ccccc23)cc1]

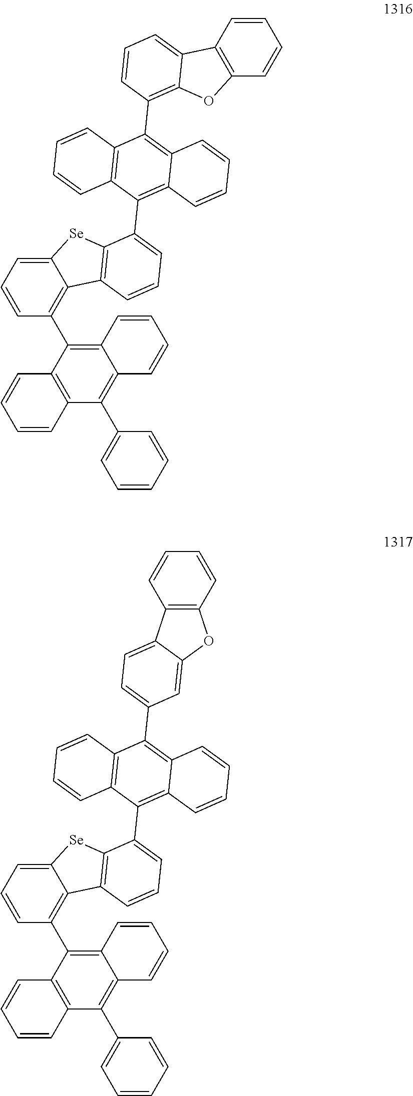 [SMILES: c1ccc(-c2c3ccccc3c(-c3cccc4[se]c5c(-c6c7ccccc7c(-c7cccc8c7oc7ccccc78)c7ccccc67)cccc5c34)c3ccccc23)cc1 ; c1ccc(-c2c3ccccc3c(-c3cccc4[se]c5c(-c6c7ccccc7c(-c7ccc8c(c7)oc7ccccc78)c7ccccc67)cccc5c34)c3ccccc23)cc1]

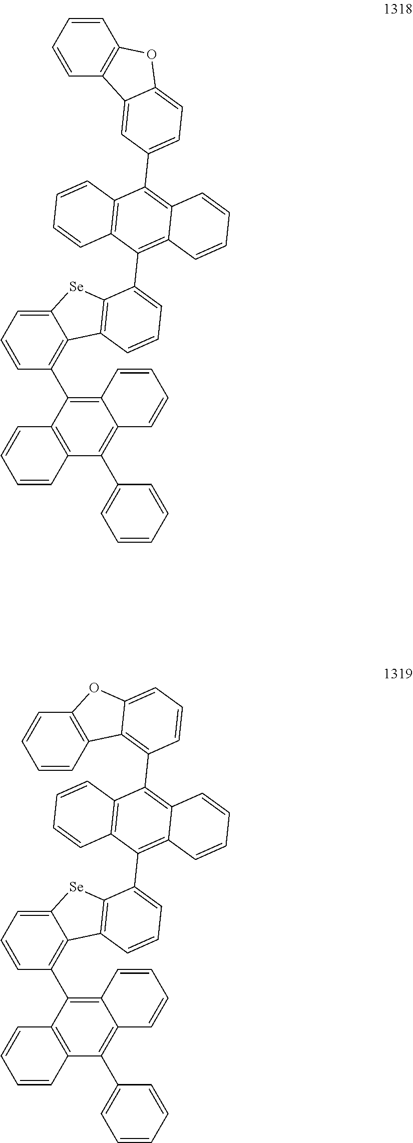 [SMILES: c1ccc(-c2c3ccccc3c(-c3cccc4[se]c5c(-c6c7ccccc7c(-c7ccc8oc9ccccc9c8c7)c7ccccc67)cccc5c34)c3ccccc23)cc1 ; c1ccc(-c2c3ccccc3c(-c3cccc4[se]c5c(-c6c7ccccc7c(-c7cccc8oc9ccccc9c78)c7ccccc67)cccc5c34)c3ccccc23)cc1]

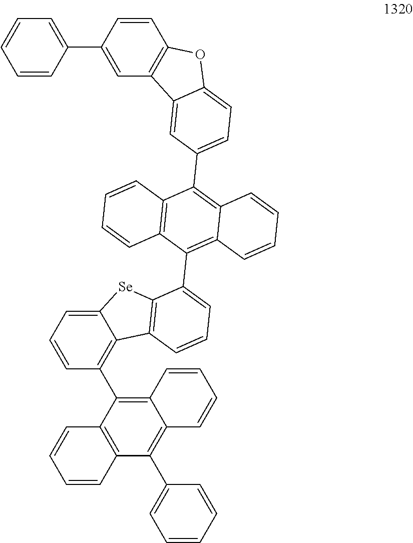 [SMILES: c1ccc(-c2ccc3oc4ccc(-c5c6ccccc6c(-c6cccc7c6[se]c6cccc(-c8c9ccccc9c(-c9ccccc9)c9ccccc89)c67)c6ccccc56)cc4c3c2)cc1]

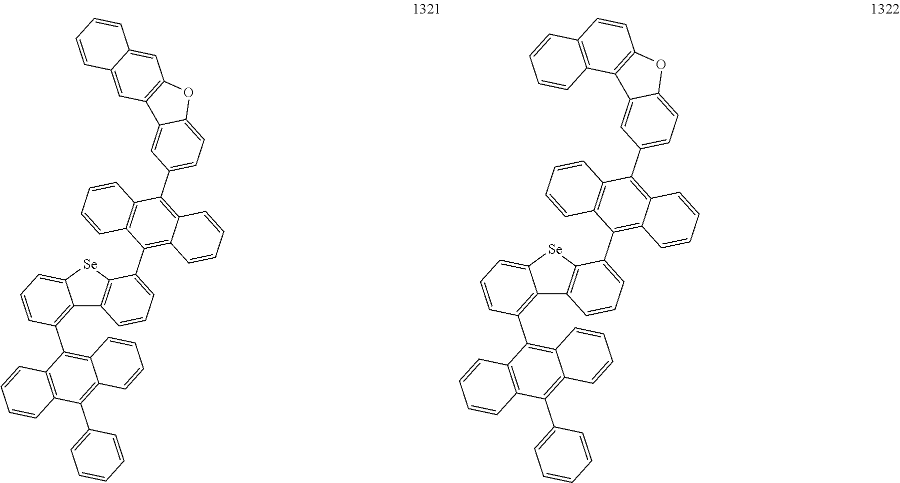 [SMILES: c1ccc(-c2c3ccccc3c(-c3cccc4[se]c5c(-c6c7ccccc7c(-c7ccc8oc9cc%10ccccc%10cc9c8c7)c7ccccc67)cccc5c34)c3ccccc23)cc1 ; c1ccc(-c2c3ccccc3c(-c3cccc4[se]c5c(-c6c7ccccc7c(-c7ccc8oc9ccc%10ccccc%10c9c8c7)c7ccccc67)cccc5c34)c3ccccc23)cc1]

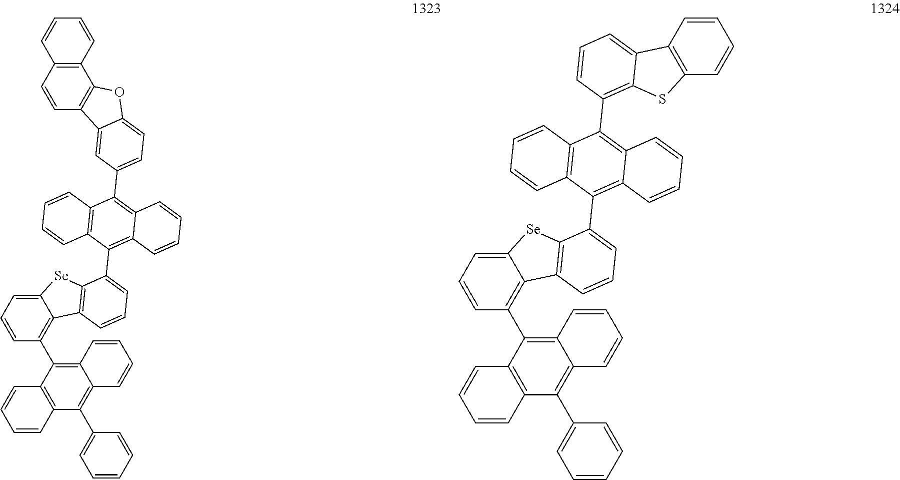 [SMILES: c1ccc(-c2c3ccccc3c(-c3cccc4[se]c5c(-c6c7ccccc7c(-c7ccc8oc9c%10ccccc%10ccc9c8c7)c7ccccc67)cccc5c34)c3ccccc23)cc1 ; c1ccc(-c2c3ccccc3c(-c3cccc4[se]c5c(-c6c7ccccc7c(-c7cccc8c7sc7ccccc78)c7ccccc67)cccc5c34)c3ccccc23)cc1]

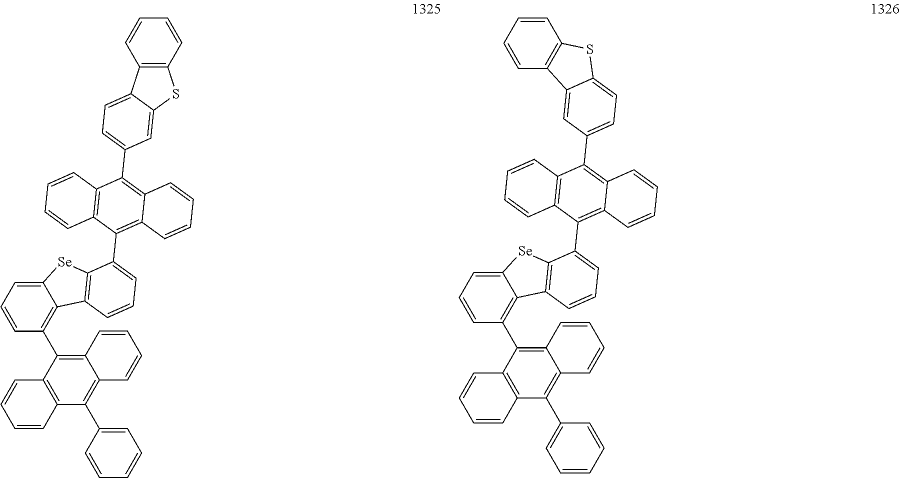 [SMILES: c1ccc(-c2c3ccccc3c(-c3cccc4[se]c5c(-c6c7ccccc7c(-c7ccc8c(c7)sc7ccccc78)c7ccccc67)cccc5c34)c3ccccc23)cc1 ; c1ccc(-c2c3ccccc3c(-c3cccc4[se]c5c(-c6c7ccccc7c(-c7ccc8sc9ccccc9c8c7)c7ccccc67)cccc5c34)c3ccccc23)cc1]

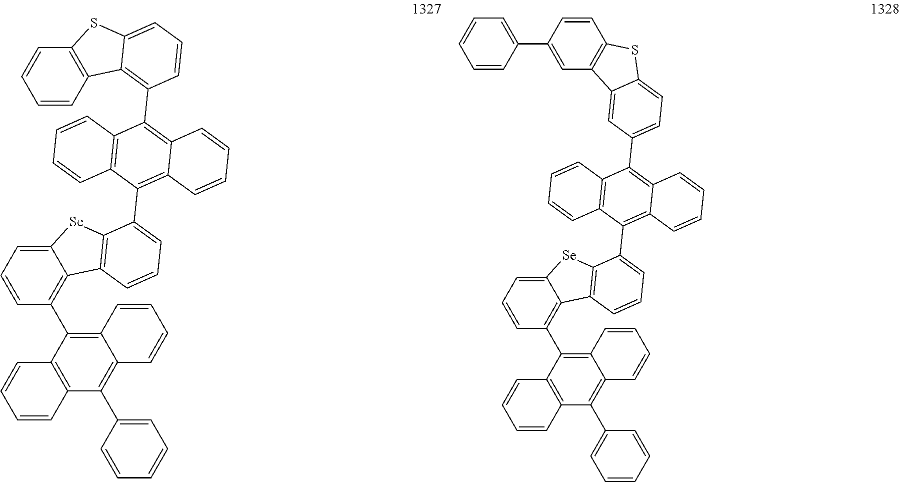 [SMILES: c1ccc(-c2c3ccccc3c(-c3cccc4[se]c5c(-c6c7ccccc7c(-c7cccc8sc9ccccc9c78)c7ccccc67)cccc5c34)c3ccccc23)cc1 ; c1ccc(-c2ccc3sc4ccc(-c5c6ccccc6c(-c6cccc7c6[se]c6cccc(-c8c9ccccc9c(-c9ccccc9)c9ccccc89)c67)c6ccccc56)cc4c3c2)cc1]

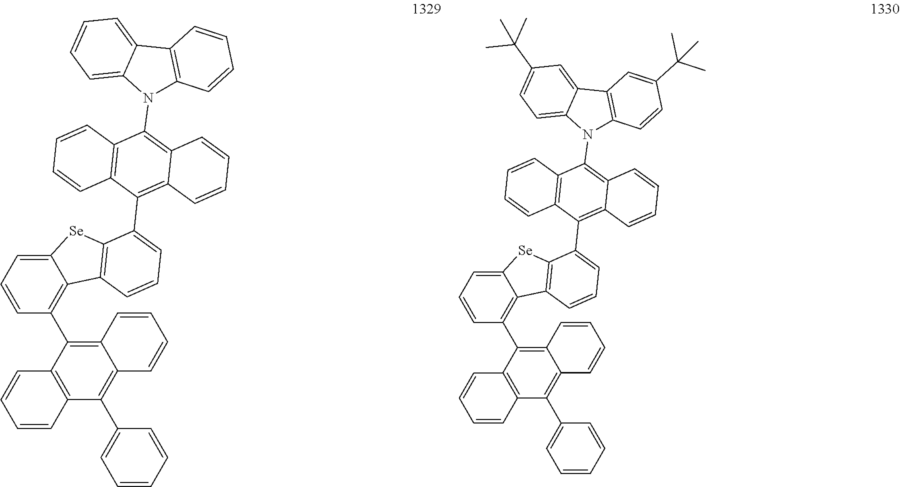 [SMILES: c1ccc(-c2c3ccccc3c(-c3cccc4[se]c5c(-c6c7ccccc7c(-n7c8ccccc8c8ccccc87)c7ccccc67)cccc5c34)c3ccccc23)cc1 ; CC(C)(C)c1ccc2c(c1)c1cc(C(C)(C)C)ccc1n2-c1c2ccccc2c(-c2cccc3c2[se]c2cccc(-c4c5ccccc5c(-c5ccccc5)c5ccccc45)c23)c2ccccc12]

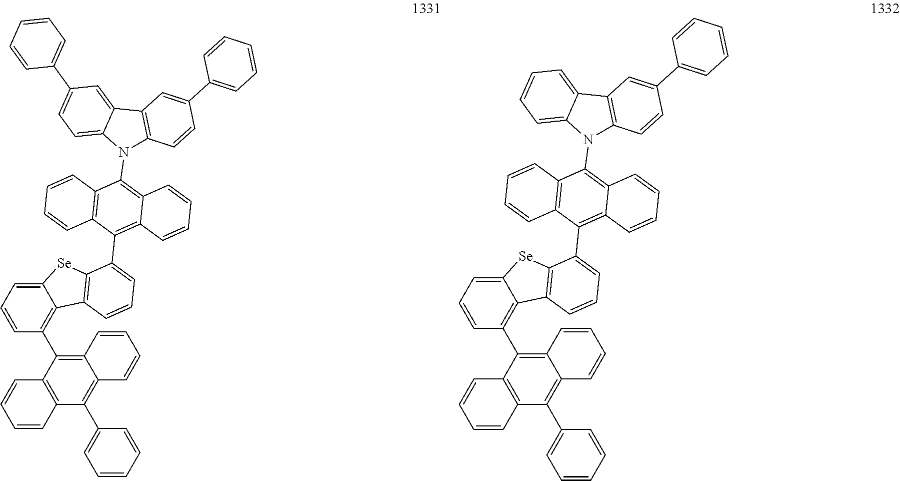 [SMILES: c1ccc(-c2ccc3c(c2)c2cc(-c4ccccc4)ccc2n3-c2c3ccccc3c(-c3cccc4c3[se]c3cccc(-c5c6ccccc6c(-c6ccccc6)c6ccccc56)c34)c3ccccc23)cc1 ; c1ccc(-c2ccc3c(c2)c2ccccc2n3-c2c3ccccc3c(-c3cccc4c3[se]c3cccc(-c5c6ccccc6c(-c6ccccc6)c6ccccc56)c34)c3ccccc23)cc1]

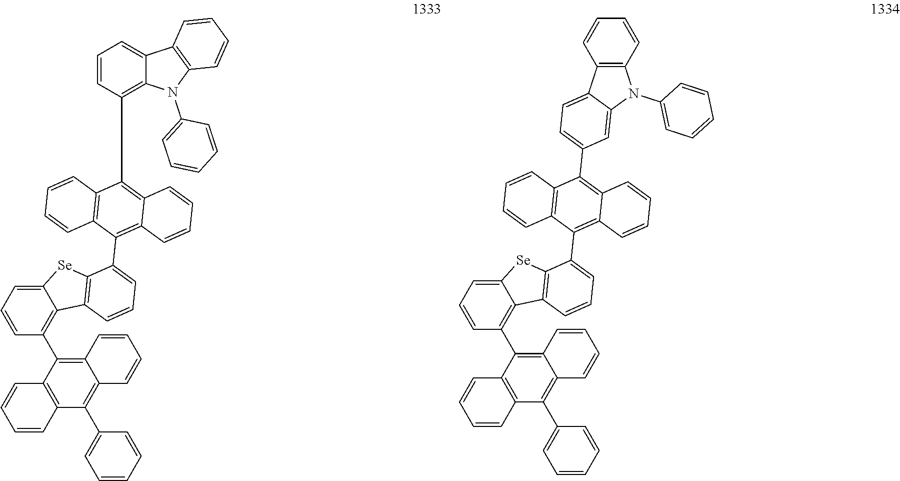 [SMILES: c1ccc(-c2c3ccccc3c(-c3cccc4[se]c5c(-c6c7ccccc7c(CCc7cccc8c9ccccc9n(-c9ccccc9)c78)c7ccccc67)cccc5c34)c3ccccc23)cc1 ; c1ccc(-c2c3ccccc3c(-c3cccc4[se]c5c(-c6c7ccccc7c(-c7ccc8c9ccccc9n(-c9ccccc9)c8c7)c7ccccc67)cccc5c34)c3ccccc23)cc1]

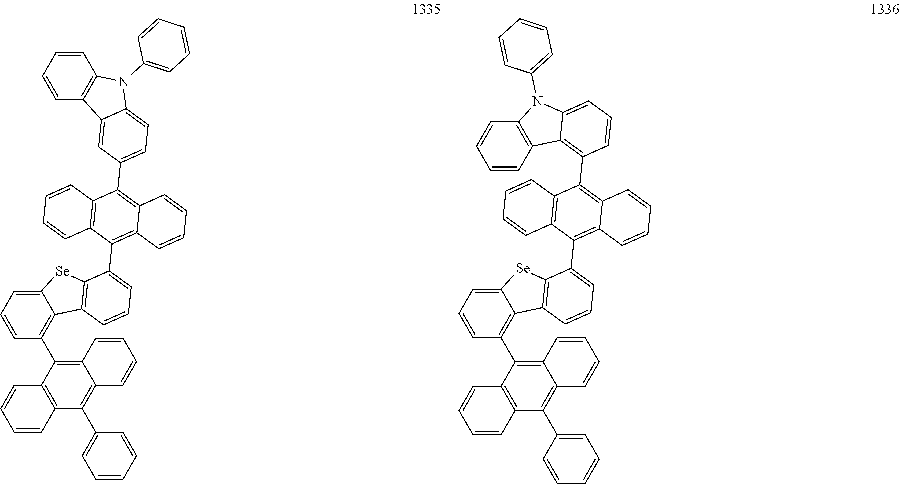 [SMILES: c1ccc(-c2c3ccccc3c(-c3cccc4[se]c5c(-c6c7ccccc7c(-c7ccc8c(c7)c7ccccc7n8-c7ccccc7)c7ccccc67)cccc5c34)c3ccccc23)cc1 ; c1ccc(-c2c3ccccc3c(-c3cccc4[se]c5c(-c6c7ccccc7c(-c7cccc8c7c7ccccc7n8-c7ccccc7)c7ccccc67)cccc5c34)c3ccccc23)cc1]

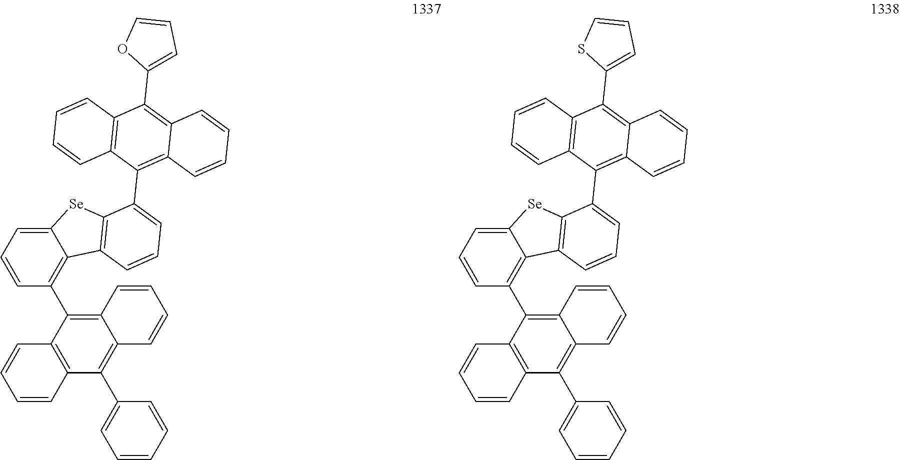 [SMILES: c1ccc(-c2c3ccccc3c(-c3cccc4[se]c5c(-c6c7ccccc7c(-c7ccco7)c7ccccc67)cccc5c34)c3ccccc23)cc1 ; c1ccc(-c2c3ccccc3c(-c3cccc4[se]c5c(-c6c7ccccc7c(-c7cccs7)c7ccccc67)cccc5c34)c3ccccc23)cc1]

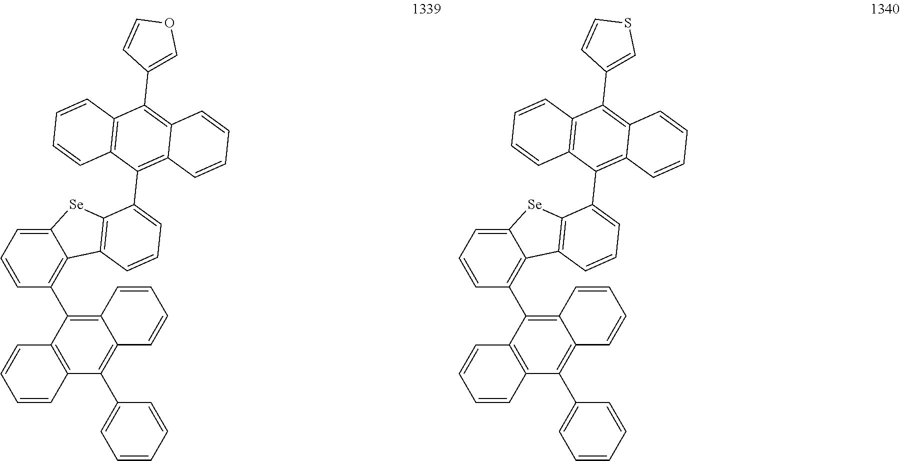 [SMILES: c1ccc(-c2c3ccccc3c(-c3cccc4[se]c5c(-c6c7ccccc7c(-c7ccoc7)c7ccccc67)cccc5c34)c3ccccc23)cc1 ; c1ccc(-c2c3ccccc3c(-c3cccc4[se]c5c(-c6c7ccccc7c(-c7ccsc7)c7ccccc67)cccc5c34)c3ccccc23)cc1]

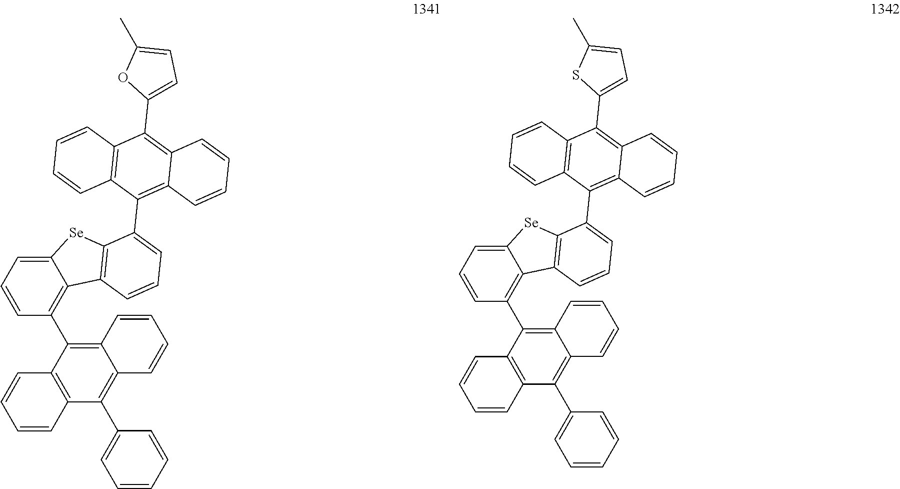 [SMILES: Cc1ccc(-c2c3ccccc3c(-c3cccc4c3[se]c3cccc(-c5c6ccccc6c(-c6ccccc6)c6ccccc56)c34)c3ccccc23)o1 ; Cc1ccc(-c2c3ccccc3c(-c3cccc4c3[se]c3cccc(-c5c6ccccc6c(-c6ccccc6)c6ccccc56)c34)c3ccccc23)s1]

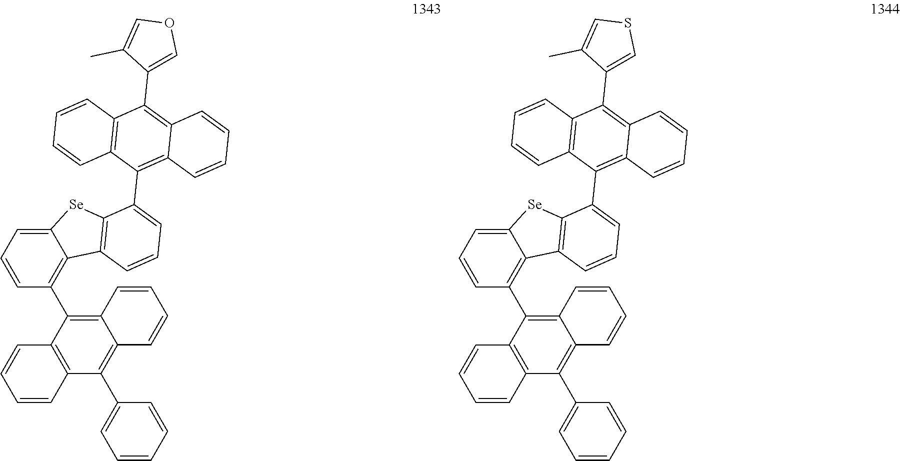 [SMILES: Cc1cocc1-c1c2ccccc2c(-c2cccc3c2[se]c2cccc(-c4c5ccccc5c(-c5ccccc5)c5ccccc45)c23)c2ccccc12 ; Cc1cscc1-c1c2ccccc2c(-c2cccc3c2[se]c2cccc(-c4c5ccccc5c(-c5ccccc5)c5ccccc45)c23)c2ccccc12]

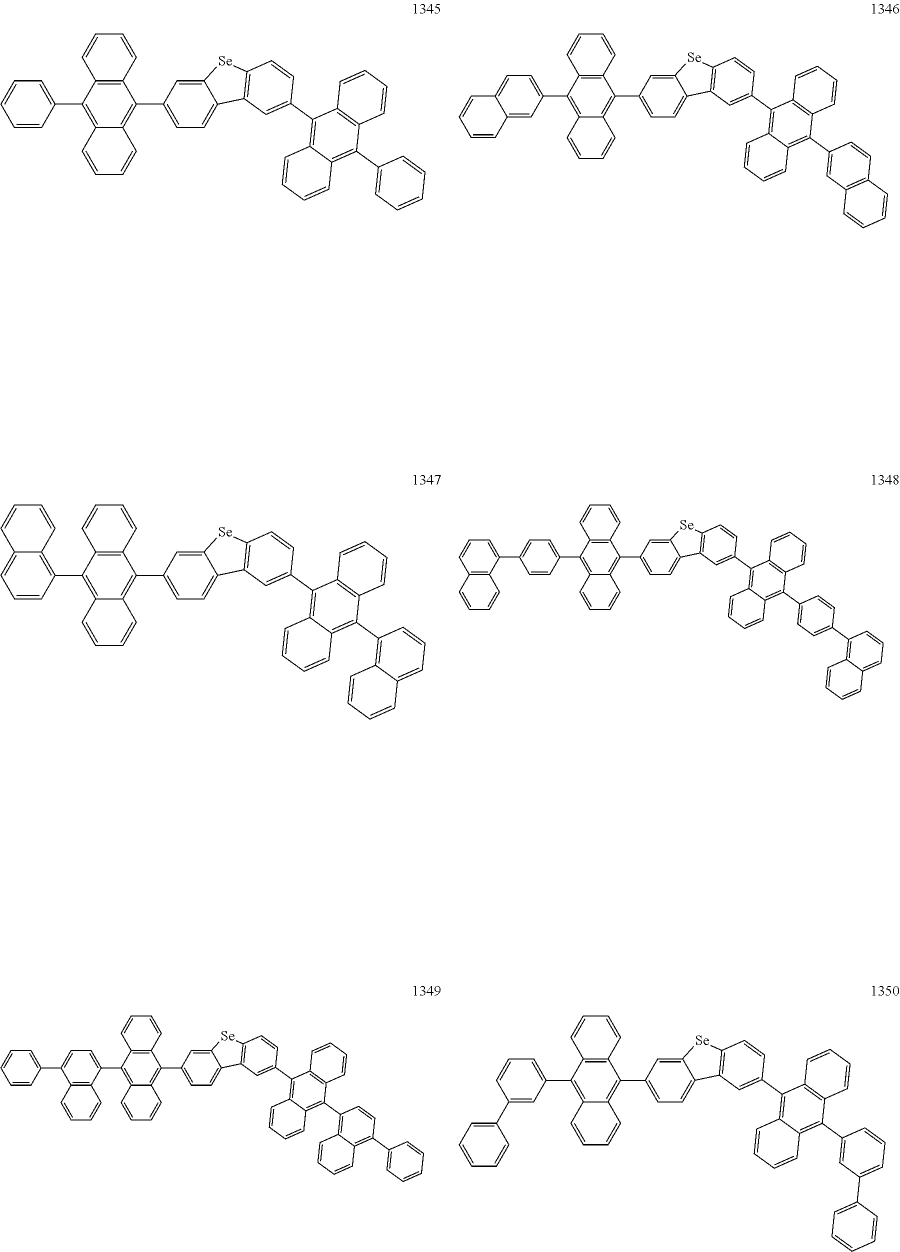

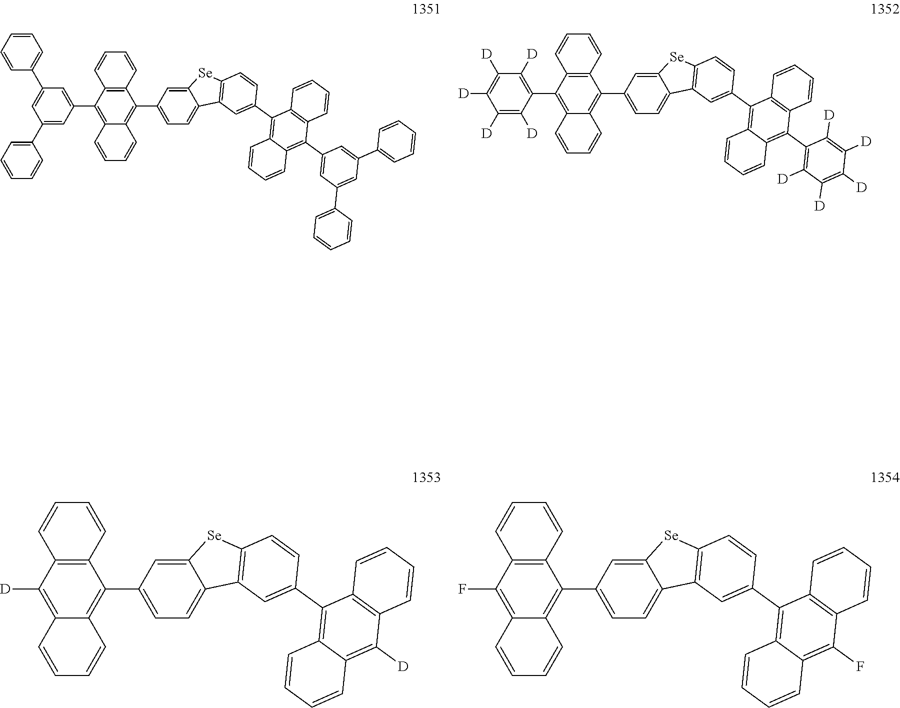 [SMILES: c1ccc(-c2cc(-c3ccccc3)cc(-c3c4ccccc4c(-c4ccc5c(c4)[se]c4ccc(-c6c7ccccc7c(-c7cc(-c8ccccc8)cc(-c8ccccc8)c7)c7ccccc67)cc45)c4ccccc34)c2)cc1 ; [2H]c1c([2H])c([2H])c(-c2c3ccccc3c(-c3ccc4c(c3)[se]c3ccc(-c5c6ccccc6c(-c6c([2H])c([2H])c([2H])c([2H])c6[2H])c6ccccc56)cc34)c3ccccc23)c([2H])c1[2H] ; [2H]c1c2ccccc2c(-c2ccc3c(c2)[se]c2ccc(-c4c5ccccc5c([2H])c5ccccc45)cc23)c2ccccc12 ; Fc1c2ccccc2c(-c2ccc3c(c2)[se]c2ccc(-c4c5ccccc5c(F)c5ccccc45)cc23)c2ccccc12]

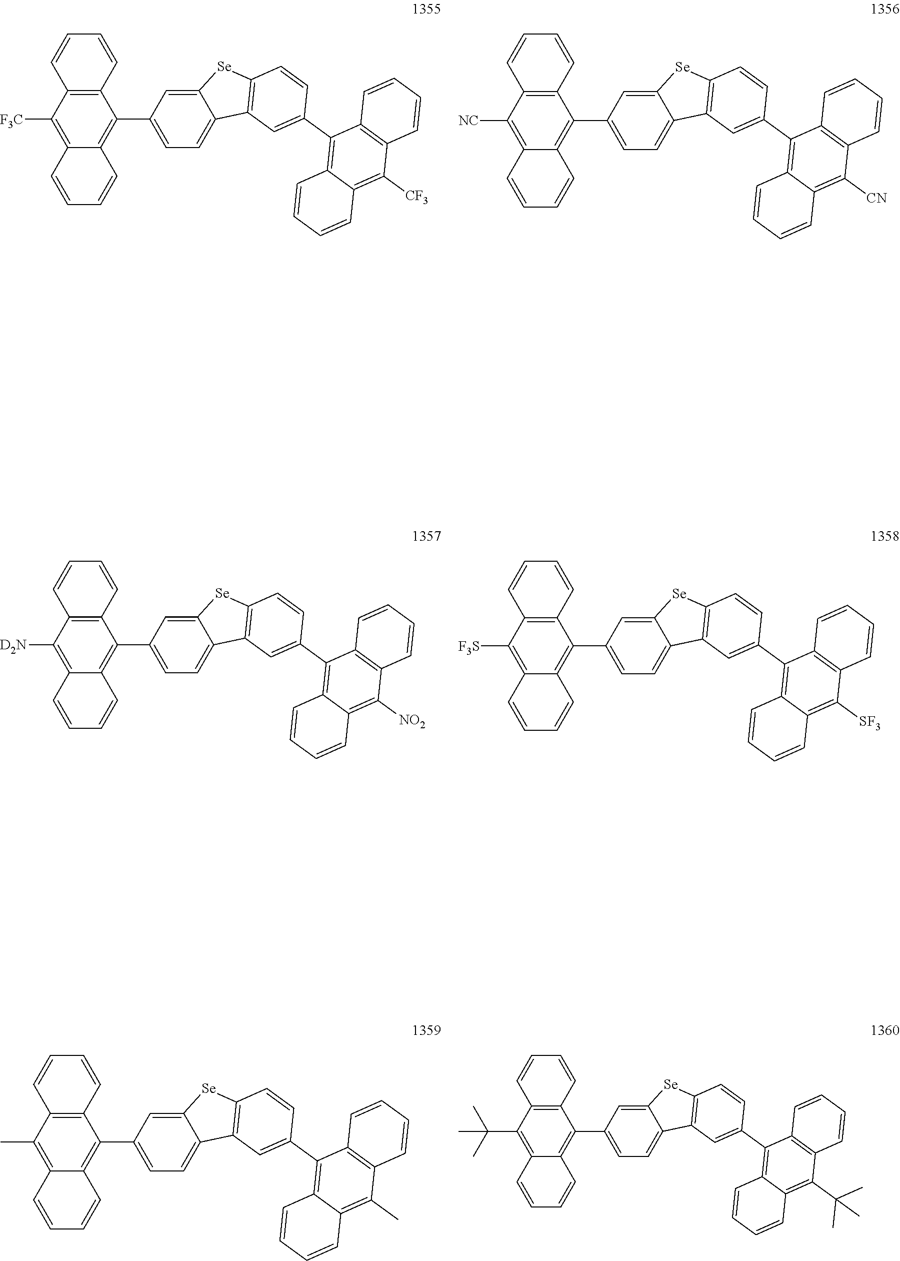

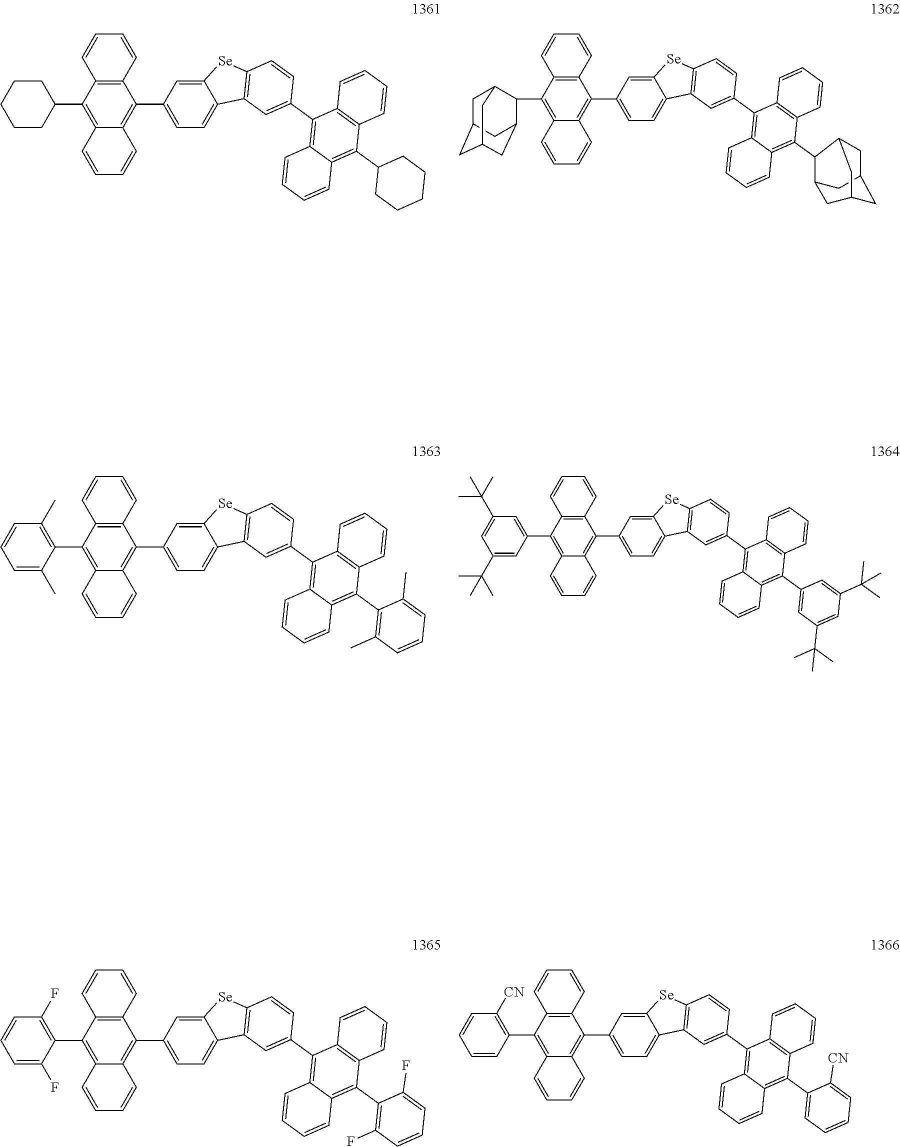

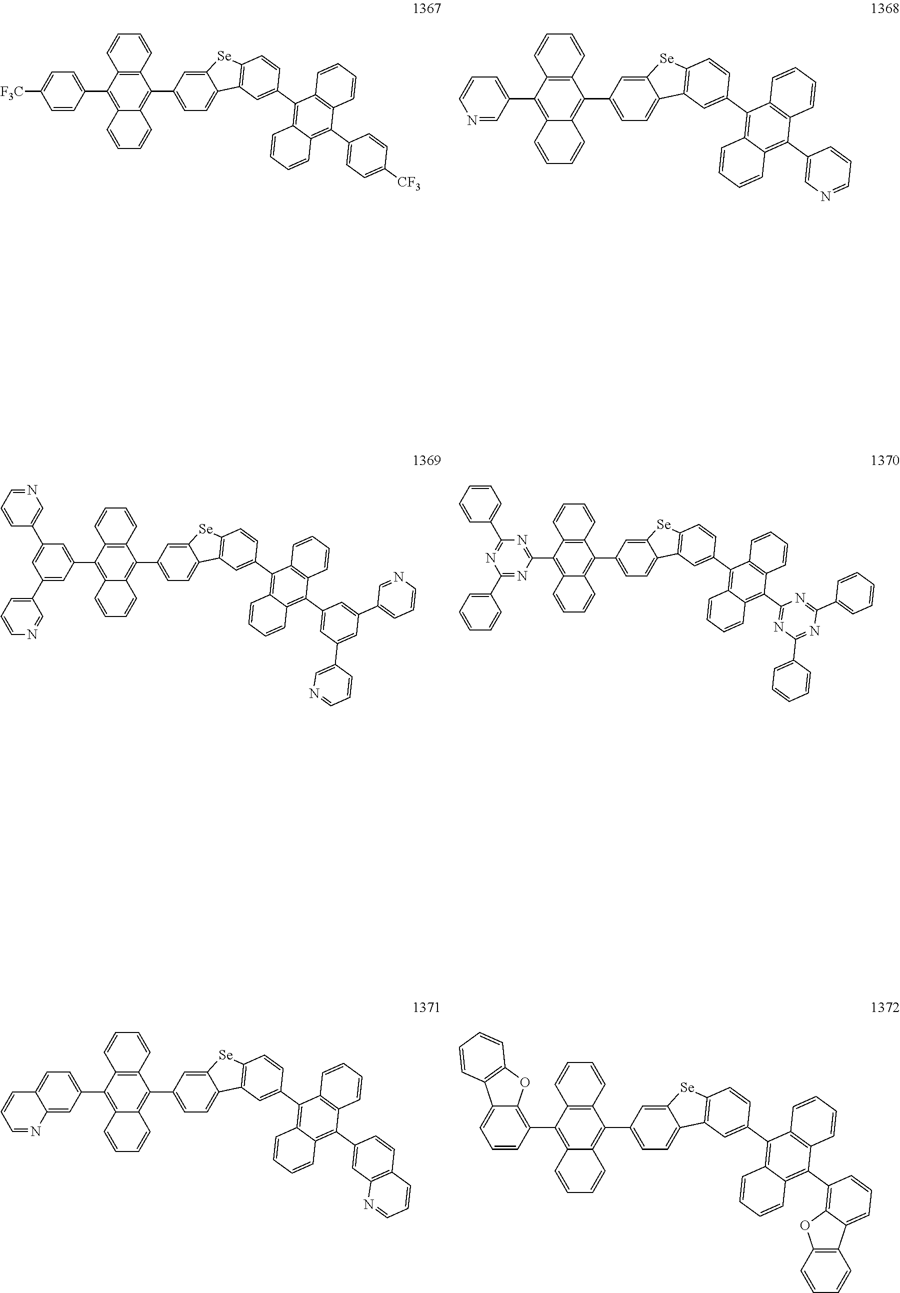

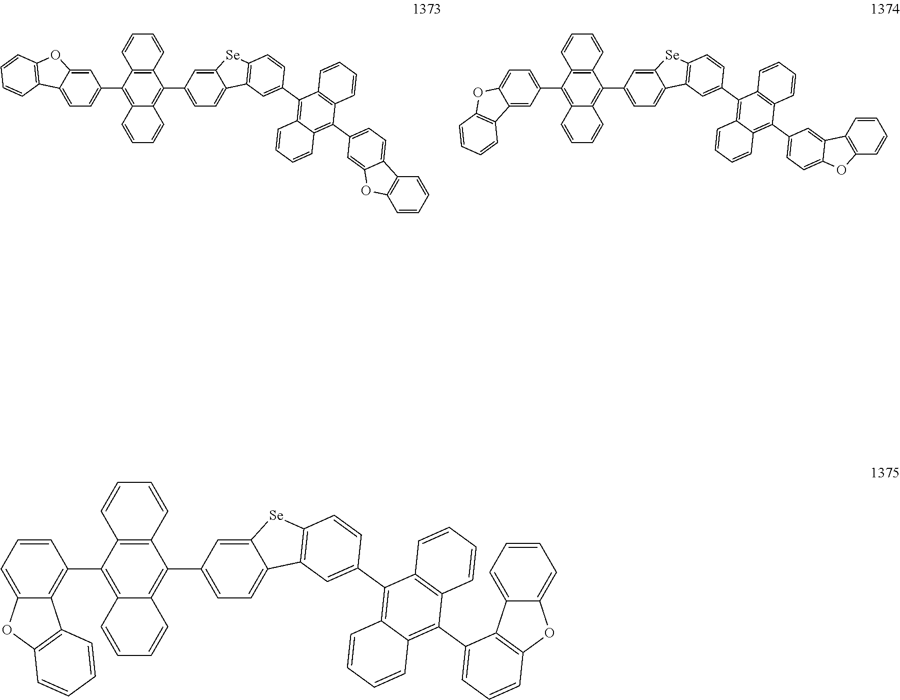 [SMILES: c1ccc2c(c1)oc1cc(-c3c4ccccc4c(-c4ccc5c(c4)[se]c4ccc(-c6c7ccccc7c(-c7ccc8c(c7)oc7ccccc78)c7ccccc67)cc45)c4ccccc34)ccc12 ; c1ccc2c(c1)oc1ccc(-c3c4ccccc4c(-c4ccc5c(c4)[se]c4ccc(-c6c7ccccc7c(-c7ccc8oc9ccccc9c8c7)c7ccccc67)cc45)c4ccccc34)cc12 ; c1ccc2c(c1)oc1cccc(-c3c4ccccc4c(-c4ccc5c(c4)[se]c4ccc(-c6c7ccccc7c(-c7cccc8oc9ccccc9c78)c7ccccc67)cc45)c4ccccc34)c12]

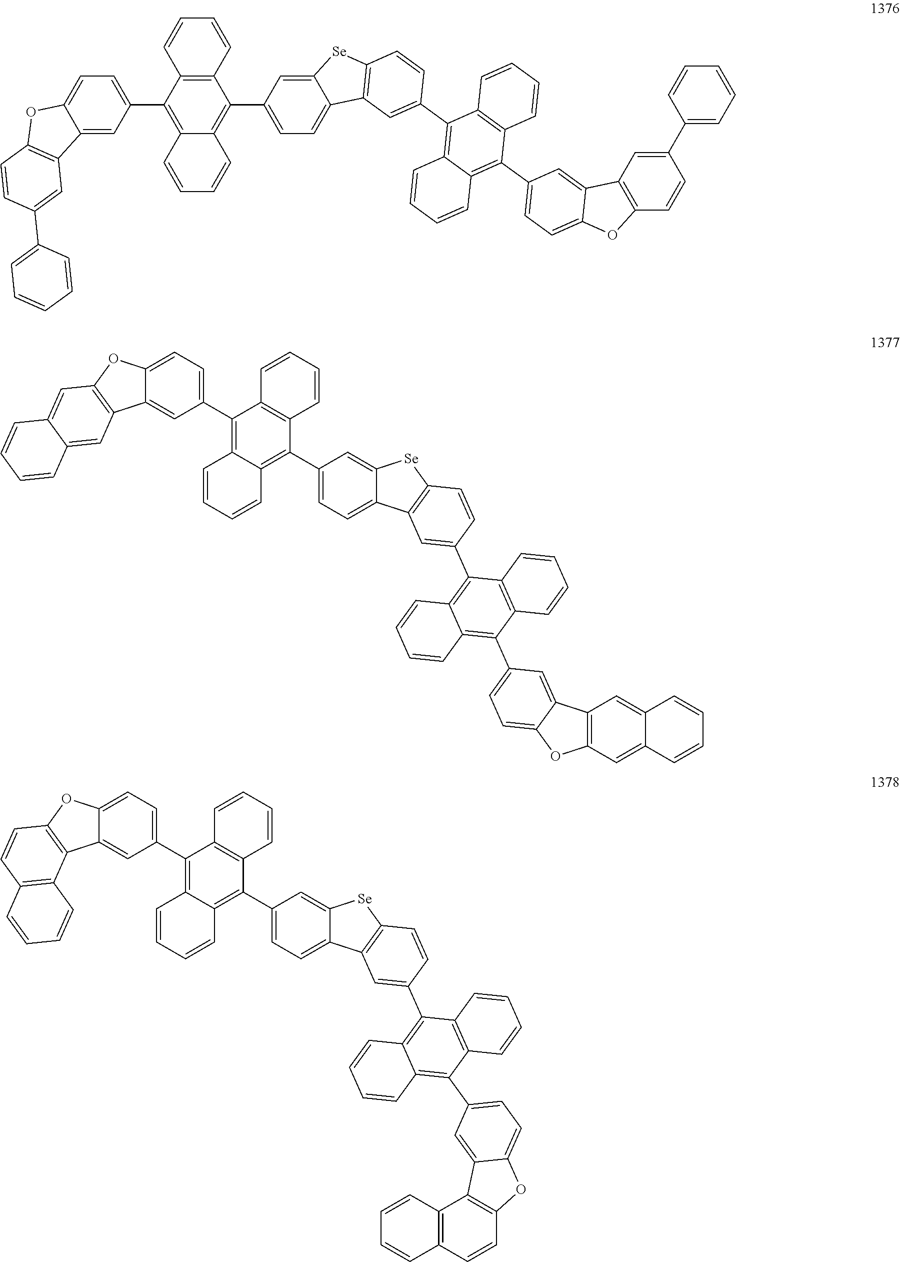 [SMILES: c1ccc(-c2ccc3oc4ccc(-c5c6ccccc6c(-c6ccc7c(c6)[se]c6ccc(-c8c9ccccc9c(-c9ccc%10oc%11ccc(-c%12ccccc%12)cc%11c%10c9)c9ccccc89)cc67)c6ccccc56)cc4c3c2)cc1 ; c1ccc2cc3c(cc2c1)oc1ccc(-c2c4ccccc4c(-c4ccc5c(c4)[se]c4ccc(-c6c7ccccc7c(-c7ccc8oc9cc%10ccccc%10cc9c8c7)c7ccccc67)cc45)c4ccccc24)cc13 ; c1ccc2c(c1)ccc1oc3ccc(-c4c5ccccc5c(-c5ccc6c(c5)[se]c5ccc(-c7c8ccccc8c(-c8ccc9oc%10ccc%11ccccc%11c%10c9c8)c8ccccc78)cc56)c5ccccc45)cc3c12]

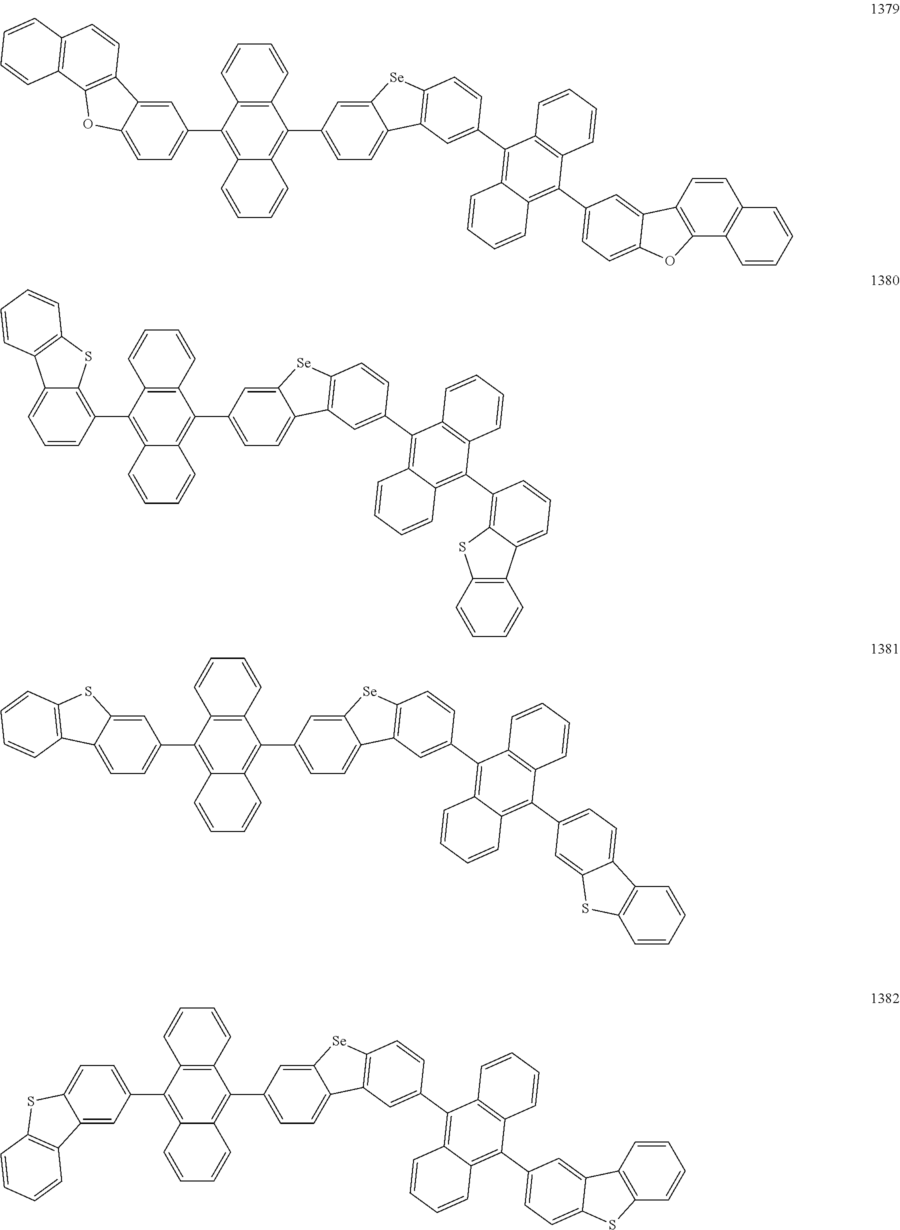 [SMILES: c1ccc2c(c1)ccc1c3cc(-c4c5ccccc5c(-c5ccc6c(c5)[se]c5ccc(-c7c8ccccc8c(-c8ccc9oc%10c%11ccccc%11ccc%10c9c8)c8ccccc78)cc56)c5ccccc45)ccc3oc21 ; c1ccc2c(c1)sc1c(-c3c4ccccc4c(-c4ccc5c(c4)[se]c4ccc(-c6c7ccccc7c(-c7cccc8c7sc7ccccc78)c7ccccc67)cc45)c4ccccc34)cccc12 ; c1ccc2c(c1)sc1cc(-c3c4ccccc4c(-c4ccc5c(c4)[se]c4ccc(-c6c7ccccc7c(-c7ccc8c(c7)sc7ccccc78)c7ccccc67)cc45)c4ccccc34)ccc12 ; c1ccc2c(c1)sc1ccc(-c3c4ccccc4c(-c4ccc5c(c4)[se]c4ccc(-c6c7ccccc7c(-c7ccc8sc9ccccc9c8c7)c7ccccc67)cc45)c4ccccc34)cc12]

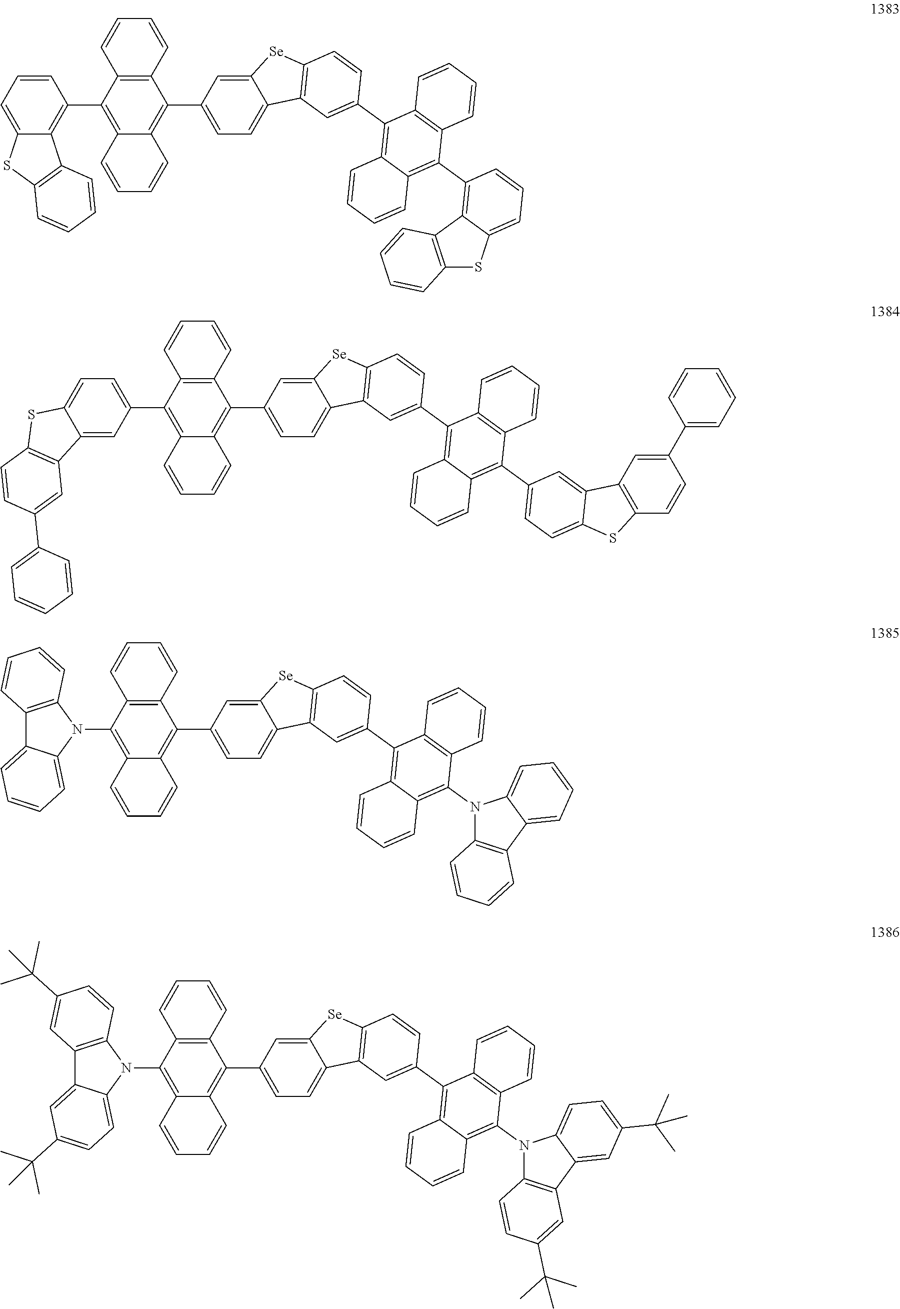

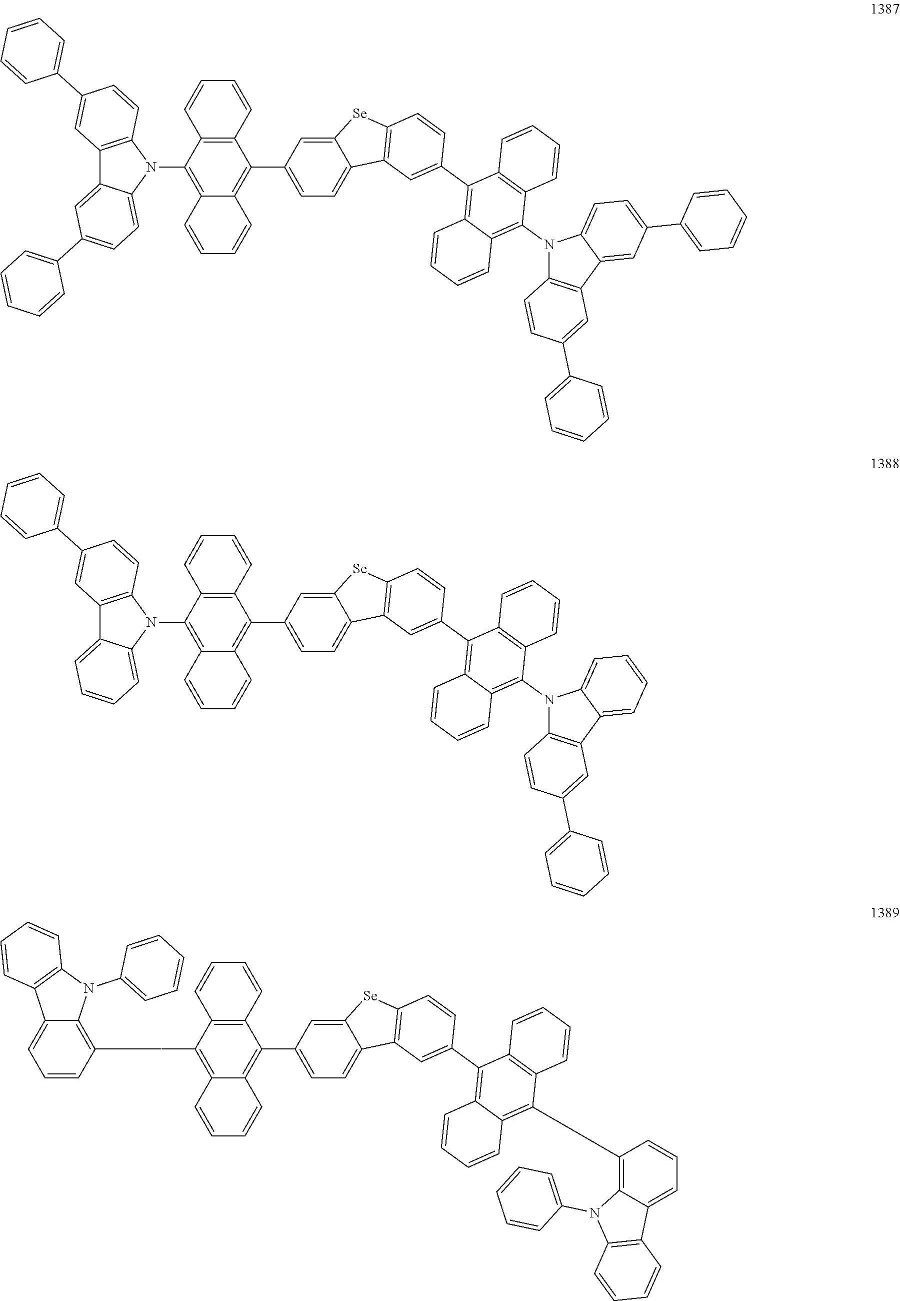 [SMILES: c1ccc(-c2ccc3c(c2)c2cc(-c4ccccc4)ccc2n3-c2c3ccccc3c(-c3ccc4c(c3)[se]c3ccc(-c5c6ccccc6c(-n6c7ccc(-c8ccccc8)cc7c7cc(-c8ccccc8)ccc76)c6ccccc56)cc34)c3ccccc23)cc1 ; c1ccc(-c2ccc3c(c2)c2ccccc2n3-c2c3ccccc3c(-c3ccc4c(c3)[se]c3ccc(-c5c6ccccc6c(-n6c7ccccc7c7cc(-c8ccccc8)ccc76)c6ccccc56)cc34)c3ccccc23)cc1 ; c1ccc(-n2c3ccccc3c3cccc(CCc4c5ccccc5c(-c5ccc6c(c5)[se]c5ccc(-c7c8ccccc8c(CCc8cccc9c%10ccccc%10n(-c%10ccccc%10)c89)c8ccccc78)cc56)c5ccccc45)c32)cc1]

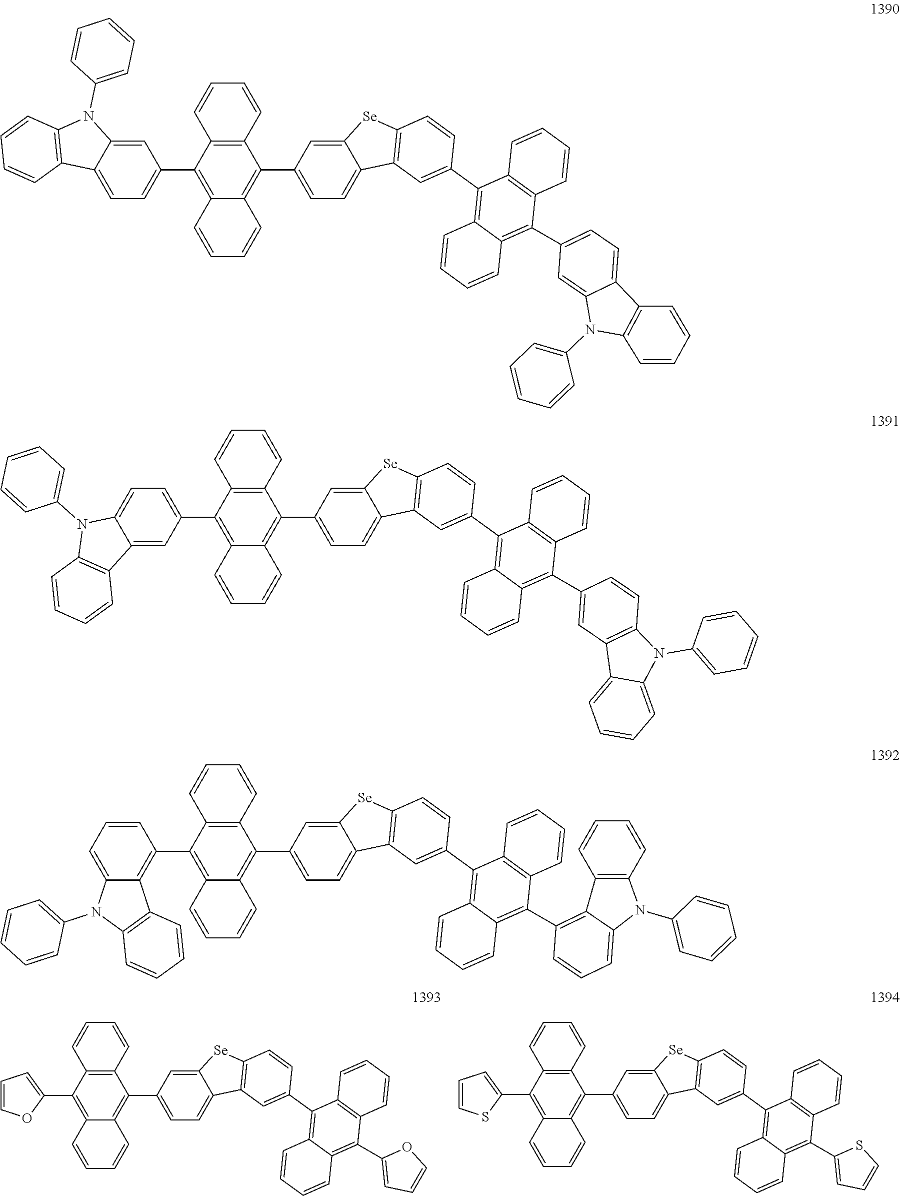

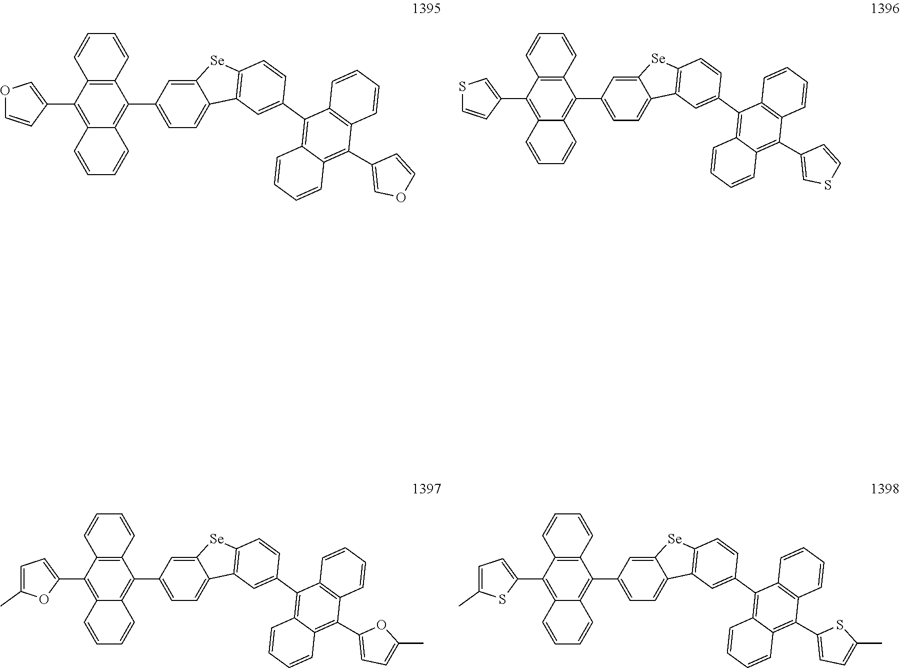

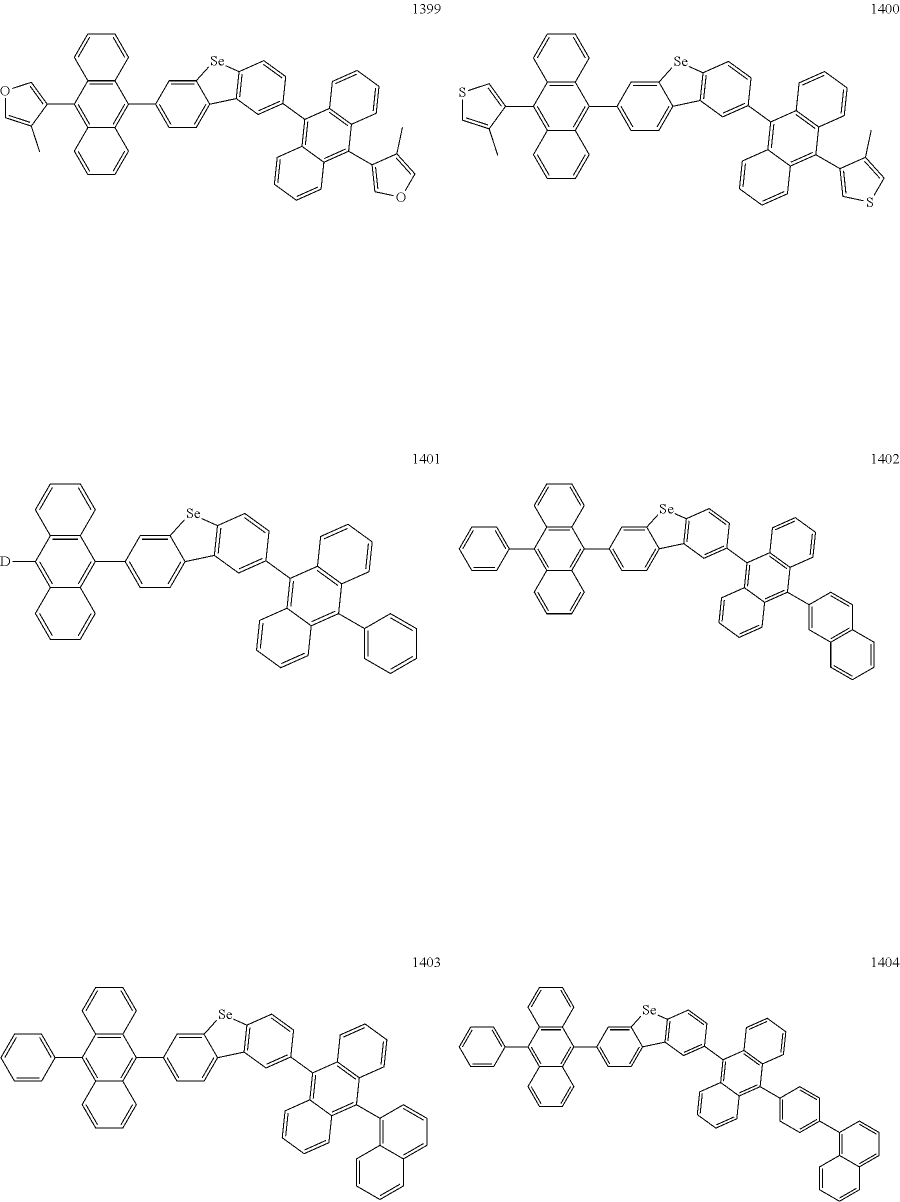

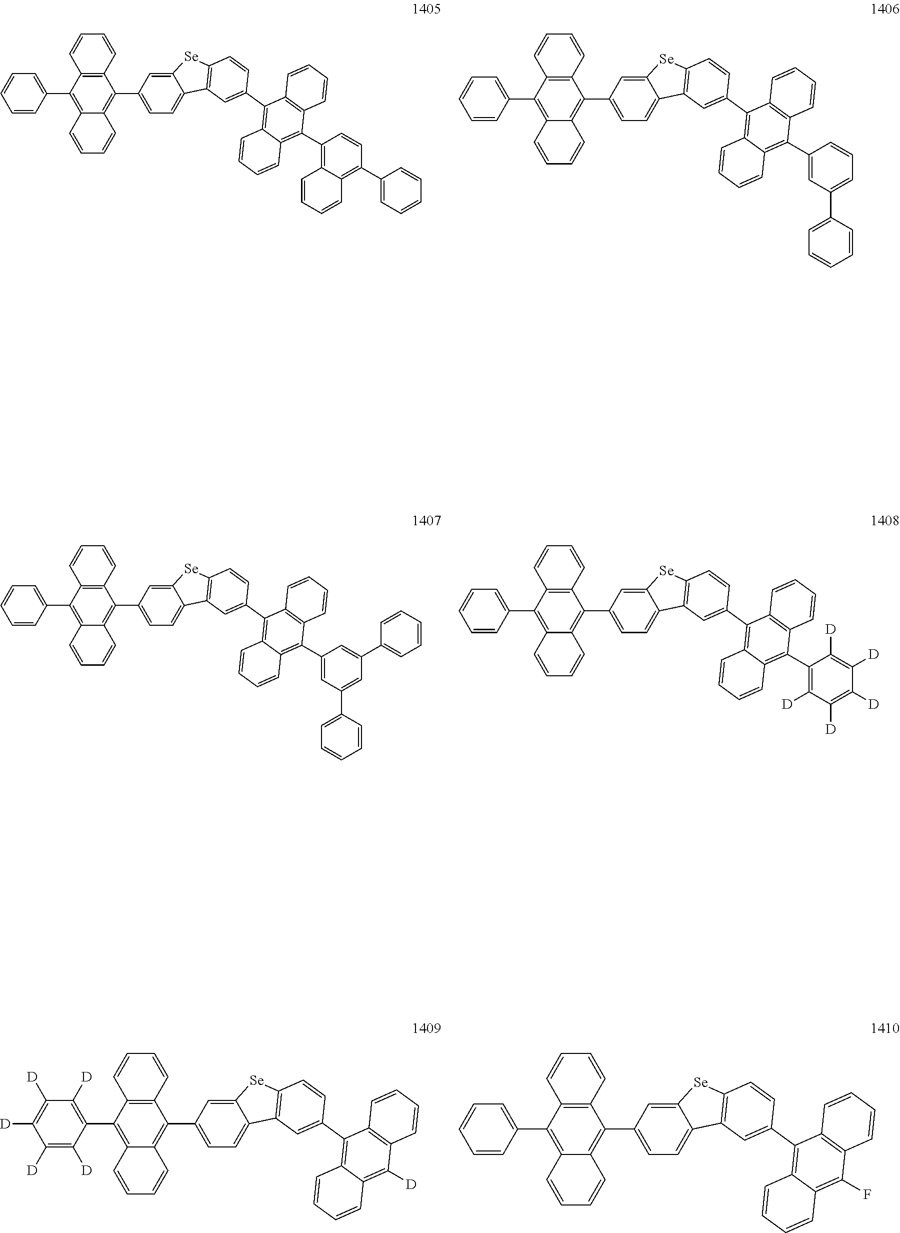

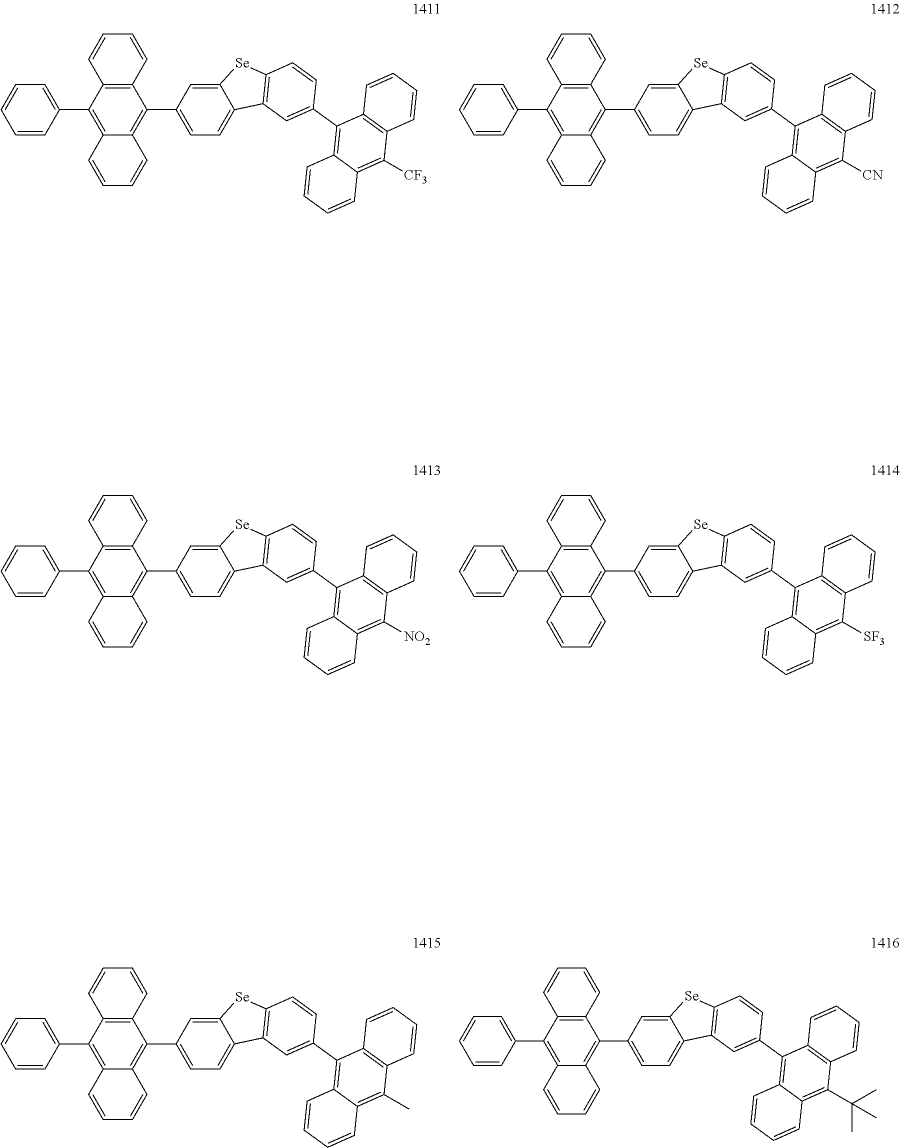

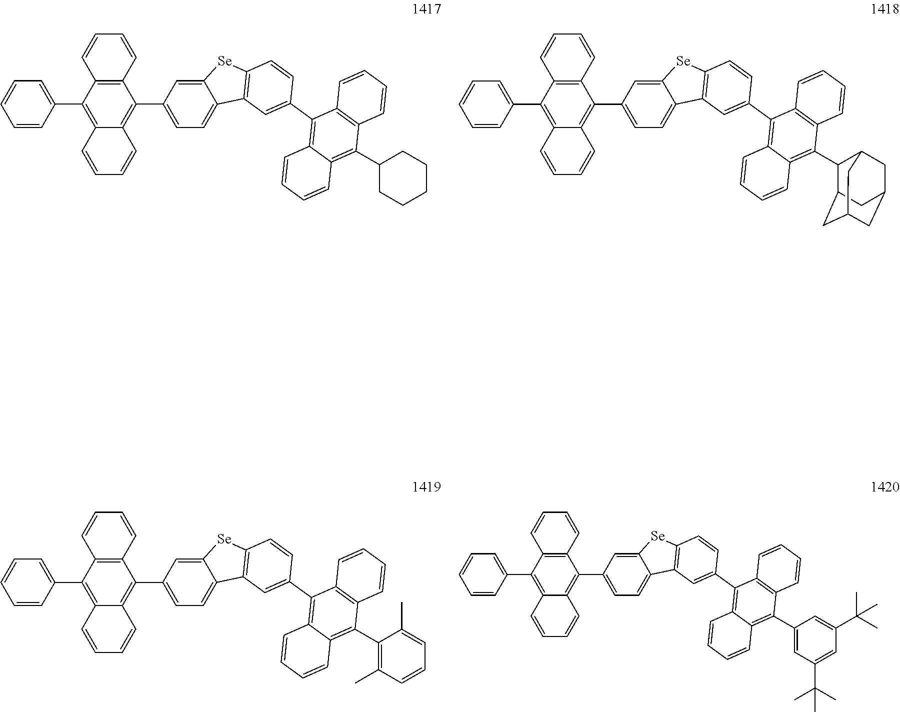 [SMILES: c1ccc(-c2c3ccccc3c(-c3ccc4c(c3)[se]c3ccc(-c5c6ccccc6c(C6CCCCC6)c6ccccc56)cc34)c3ccccc23)cc1 ; c1ccc(-c2c3ccccc3c(-c3ccc4c(c3)[se]c3ccc(-c5c6ccccc6c(C6C7CC8CC(C7)CC6C8)c6ccccc56)cc34)c3ccccc23)cc1 ; Cc1cccc(C)c1-c1c2ccccc2c(-c2ccc3[se]c4cc(-c5c6ccccc6c(-c6ccccc6)c6ccccc56)ccc4c3c2)c2ccccc12 ; CC(C)(C)c1cc(-c2c3ccccc3c(-c3ccc4[se]c5cc(-c6c7ccccc7c(-c7ccccc7)c7ccccc67)ccc5c4c3)c3ccccc23)cc(C(C)(C)C)c1]

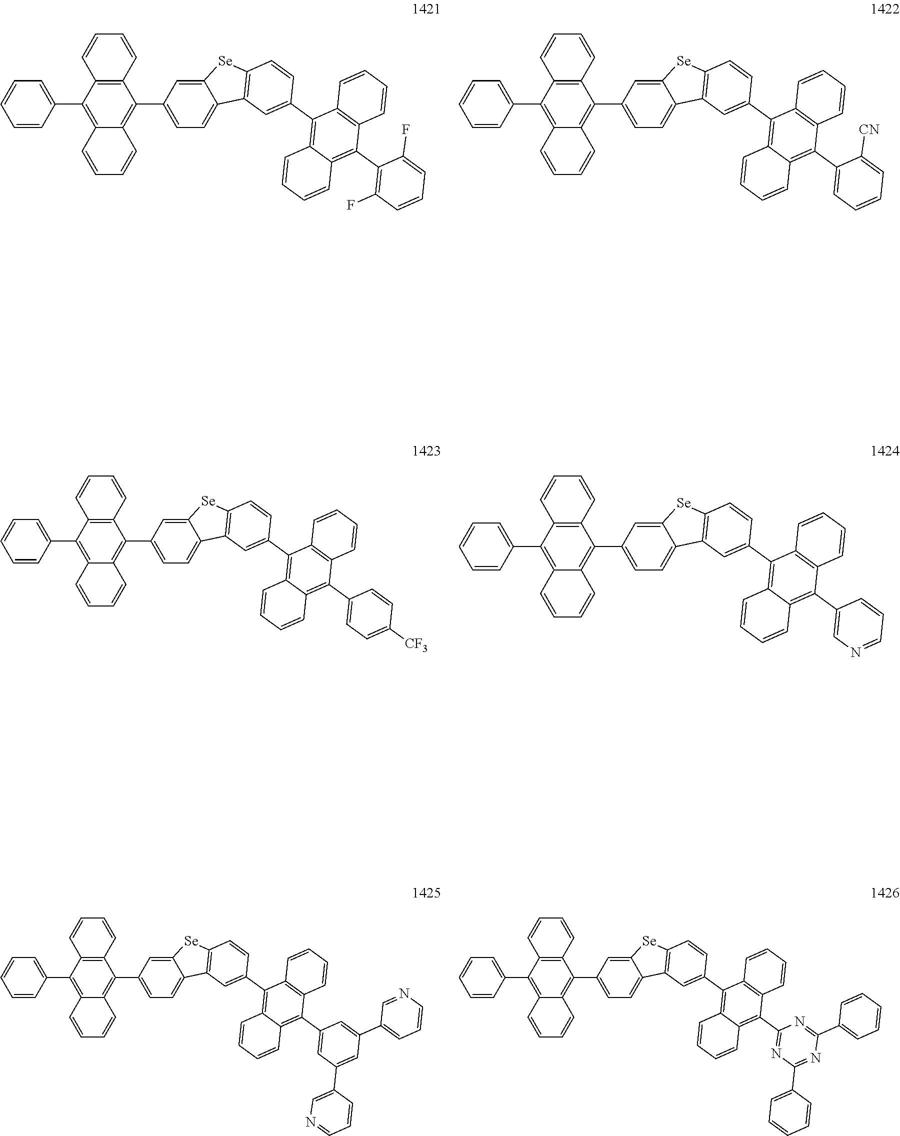

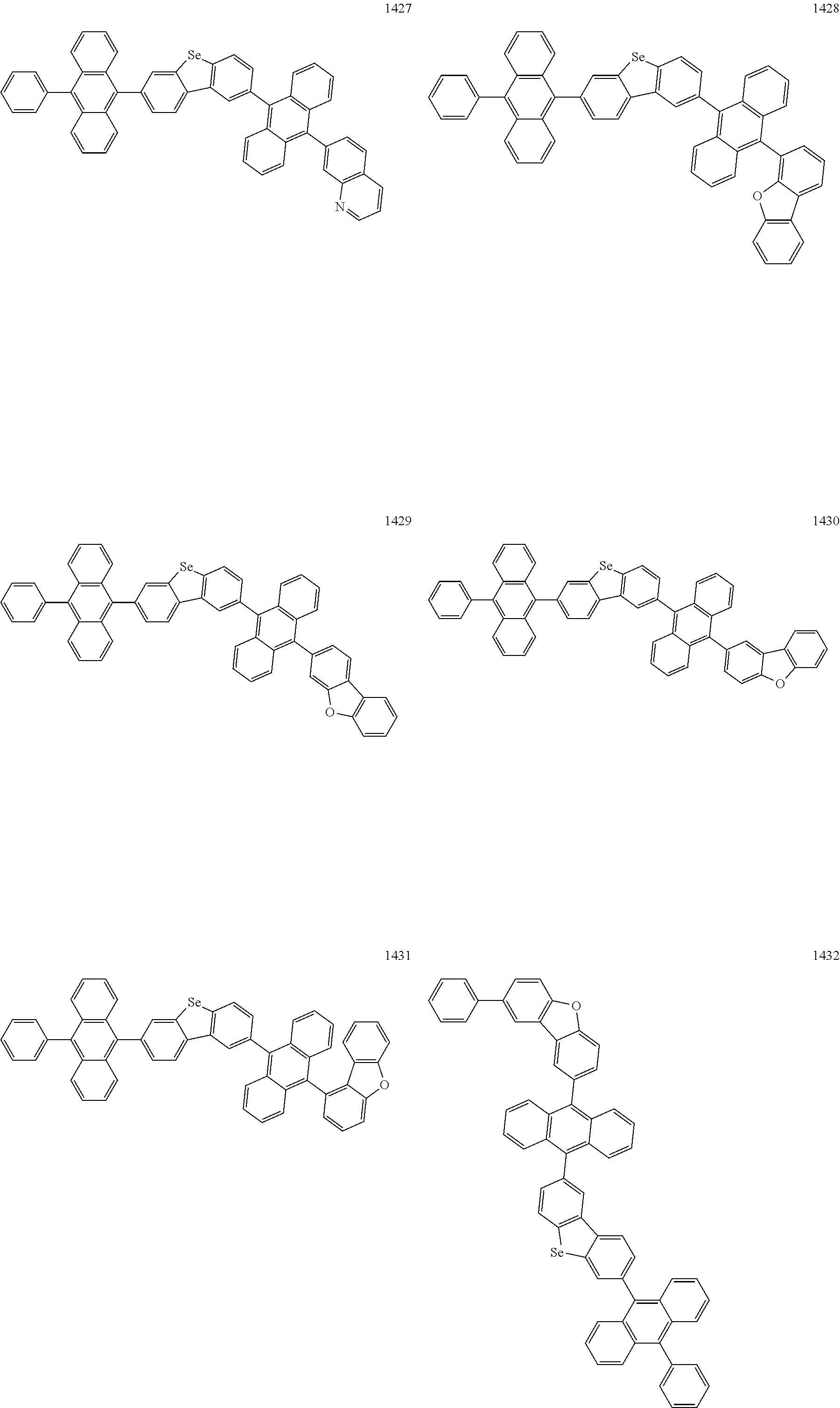

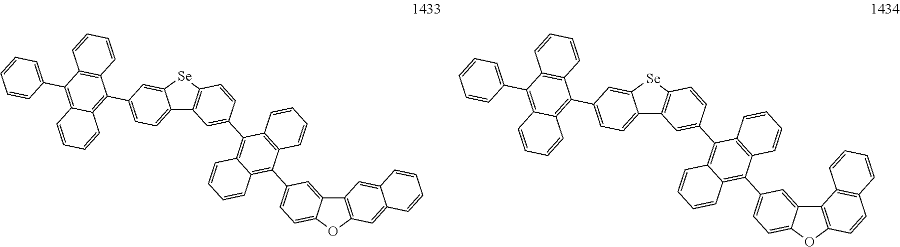 [SMILES: c1ccc(-c2c3ccccc3c(-c3ccc4c(c3)[se]c3ccc(-c5c6ccccc6c(-c6ccc7oc8cc9ccccc9cc8c7c6)c6ccccc56)cc34)c3ccccc23)cc1 ; c1ccc(-c2c3ccccc3c(-c3ccc4c(c3)[se]c3ccc(-c5c6ccccc6c(-c6ccc7oc8ccc9ccccc9c8c7c6)c6ccccc56)cc34)c3ccccc23)cc1]

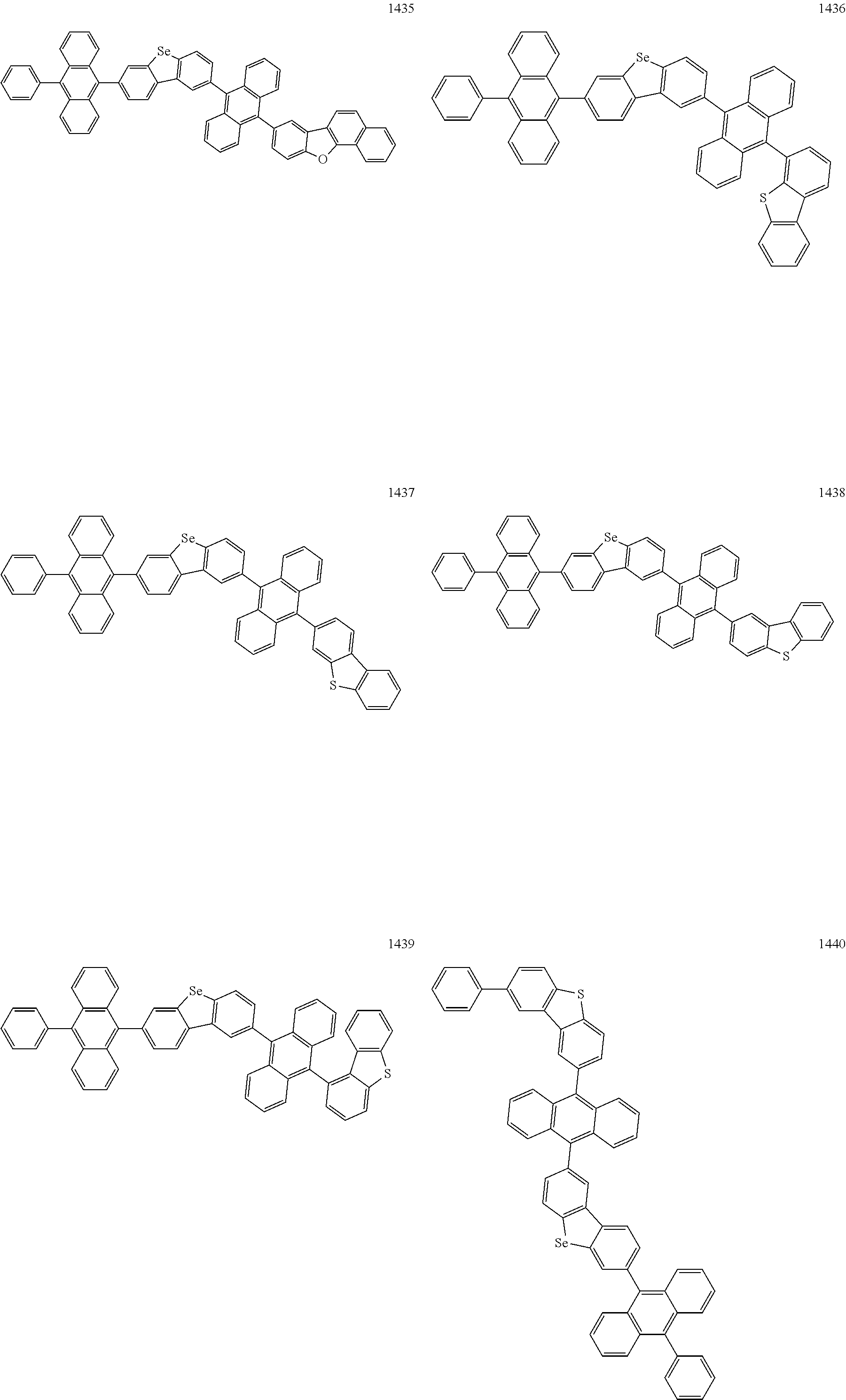

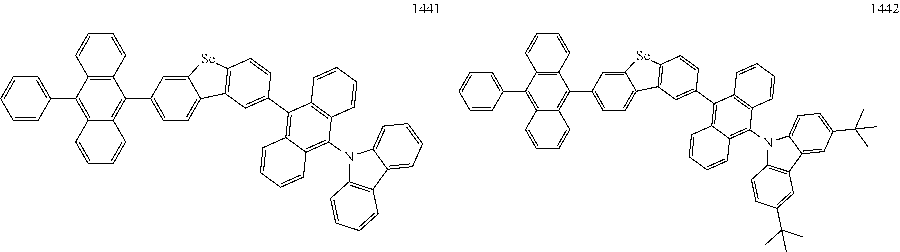 [SMILES: c1ccc(-c2c3ccccc3c(-c3ccc4c(c3)[se]c3ccc(-c5c6ccccc6c(-n6c7ccccc7c7ccccc76)c6ccccc56)cc34)c3ccccc23)cc1 ; CC(C)(C)c1ccc2c(c1)c1cc(C(C)(C)C)ccc1n2-c1c2ccccc2c(-c2ccc3[se]c4cc(-c5c6ccccc6c(-c6ccccc6)c6ccccc56)ccc4c3c2)c2ccccc12]

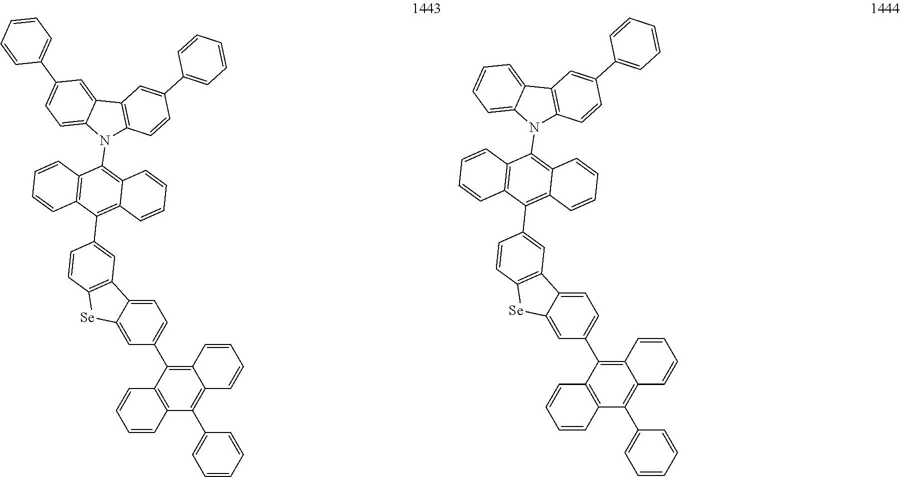 [SMILES: c1ccc(-c2ccc3c(c2)c2cc(-c4ccccc4)ccc2n3-c2c3ccccc3c(-c3ccc4[se]c5cc(-c6c7ccccc7c(-c7ccccc7)c7ccccc67)ccc5c4c3)c3ccccc23)cc1 ; c1ccc(-c2ccc3c(c2)c2ccccc2n3-c2c3ccccc3c(-c3ccc4[se]c5cc(-c6c7ccccc7c(-c7ccccc7)c7ccccc67)ccc5c4c3)c3ccccc23)cc1]

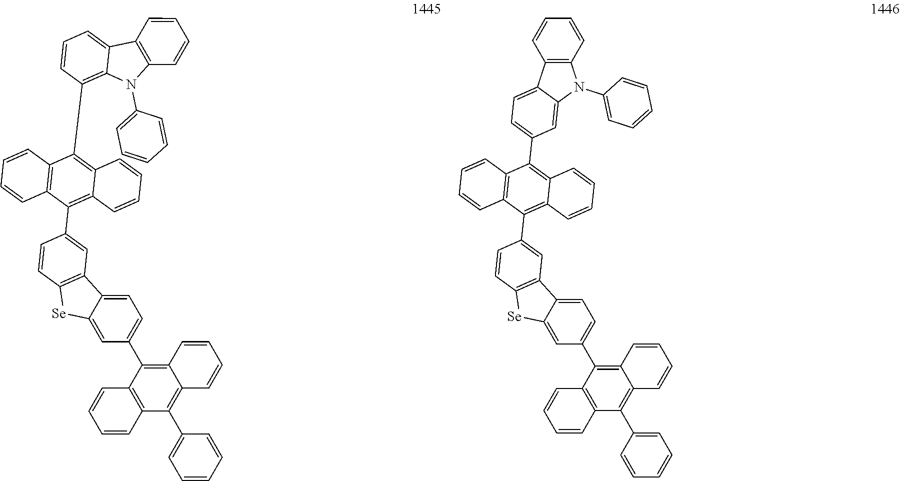 [SMILES: c1ccc(-c2c3ccccc3c(-c3ccc4c(c3)[se]c3ccc(-c5c6ccccc6c(-c6cccc7c8ccccc8n(-c8ccccc8)c67)c6ccccc56)cc34)c3ccccc23)cc1 ; c1ccc(-c2c3ccccc3c(-c3ccc4c(c3)[se]c3ccc(-c5c6ccccc6c(-c6ccc7c8ccccc8n(-c8ccccc8)c7c6)c6ccccc56)cc34)c3ccccc23)cc1]

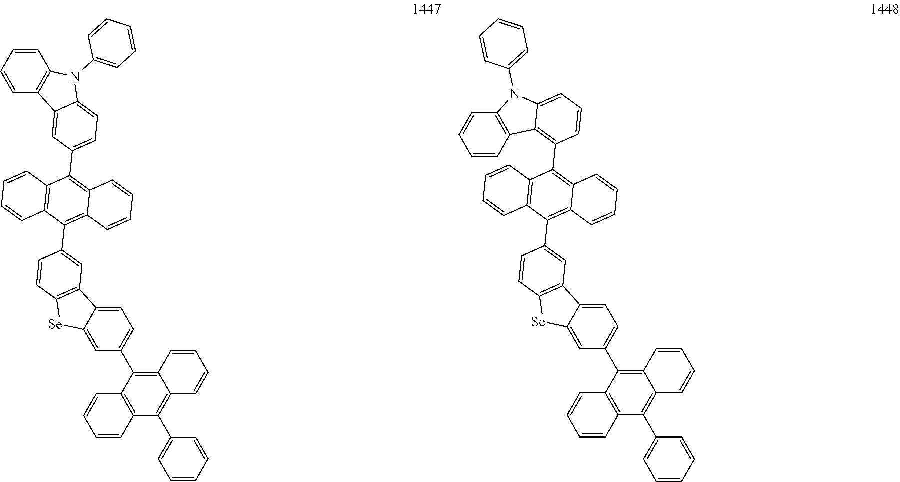 [SMILES: c1ccc(-c2c3ccccc3c(-c3ccc4c(c3)[se]c3ccc(-c5c6ccccc6c(-c6ccc7c(c6)c6ccccc6n7-c6ccccc6)c6ccccc56)cc34)c3ccccc23)cc1 ; c1ccc(-c2c3ccccc3c(-c3ccc4c(c3)[se]c3ccc(-c5c6ccccc6c(-c6cccc7c6c6ccccc6n7-c6ccccc6)c6ccccc56)cc34)c3ccccc23)cc1]

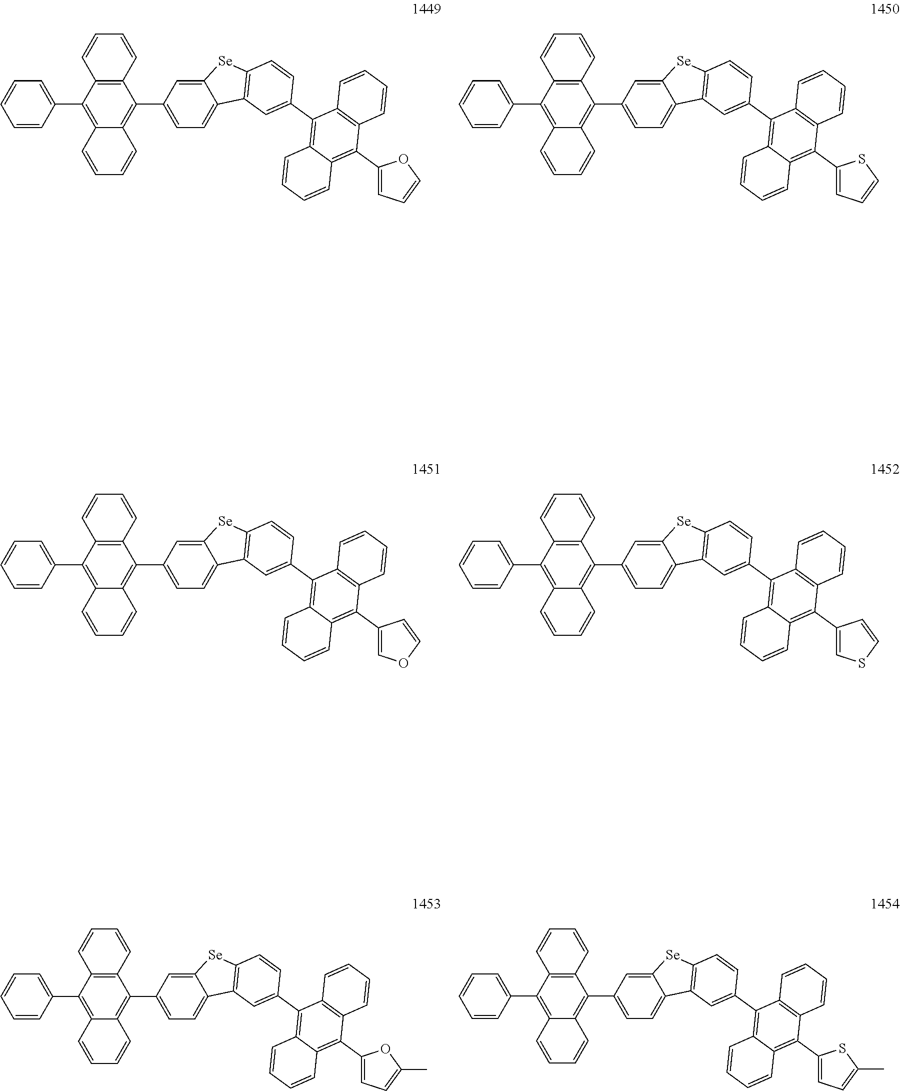

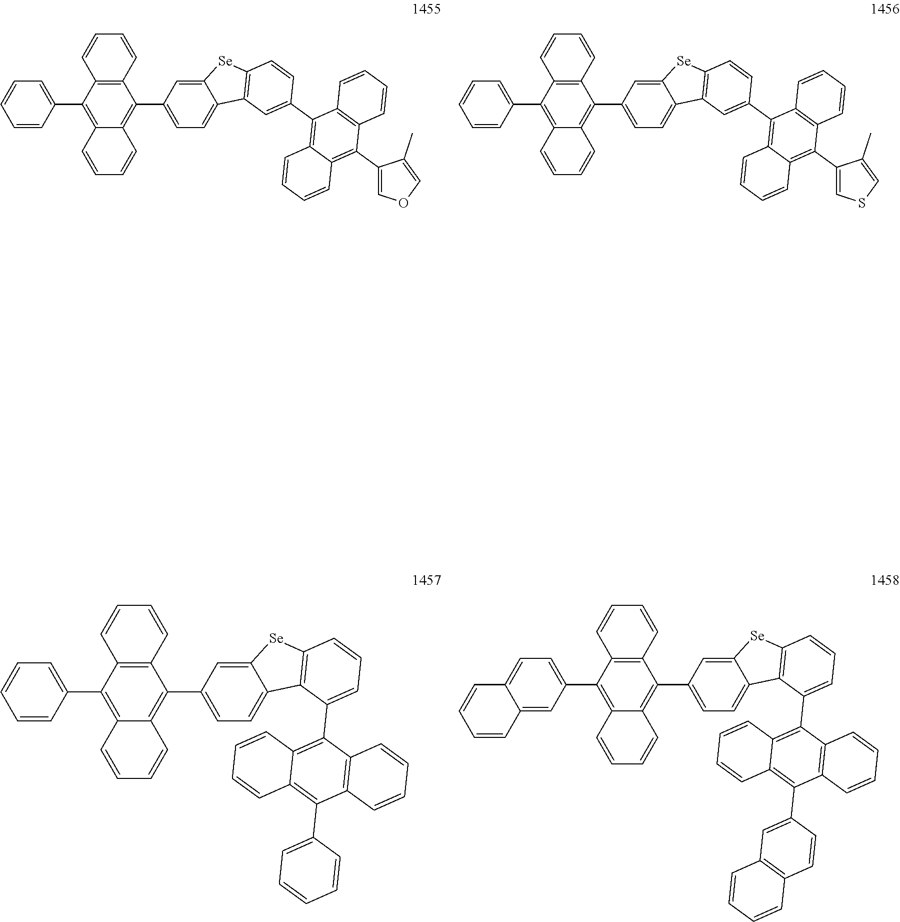 [SMILES: Cc1cocc1-c1c2ccccc2c(-c2ccc3[se]c4cc(-c5c6ccccc6c(-c6ccccc6)c6ccccc56)ccc4c3c2)c2ccccc12 ; Cc1cscc1-c1c2ccccc2c(-c2ccc3[se]c4cc(-c5c6ccccc6c(-c6ccccc6)c6ccccc56)ccc4c3c2)c2ccccc12 ; c1ccc(-c2c3ccccc3c(-c3ccc4c(c3)[se]c3cccc(-c5c6ccccc6c(-c6ccccc6)c6ccccc56)c34)c3ccccc23)cc1 ; c1ccc2cc(-c3c4ccccc4c(-c4ccc5c(c4)[se]c4cccc(-c6c7ccccc7c(-c7ccc8ccccc8c7)c7ccccc67)c45)c4ccccc34)ccc2c1]

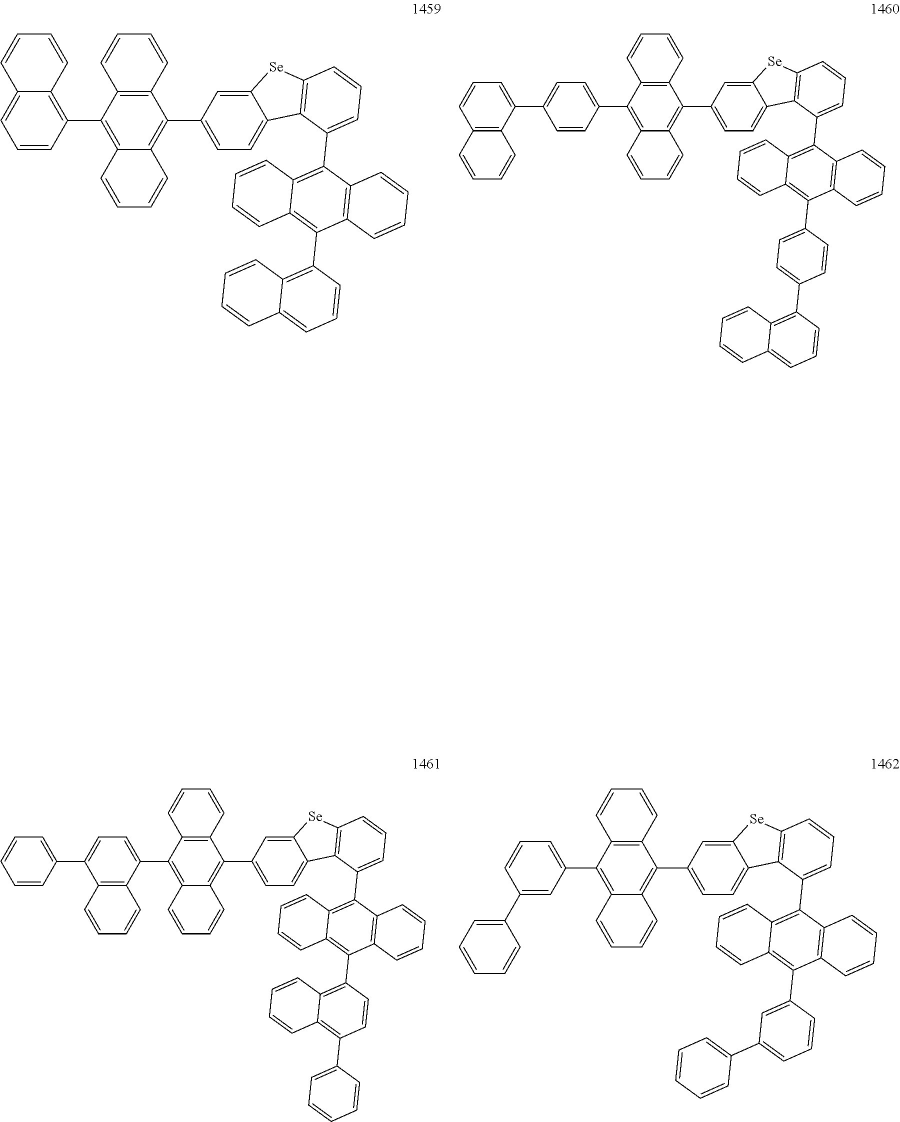

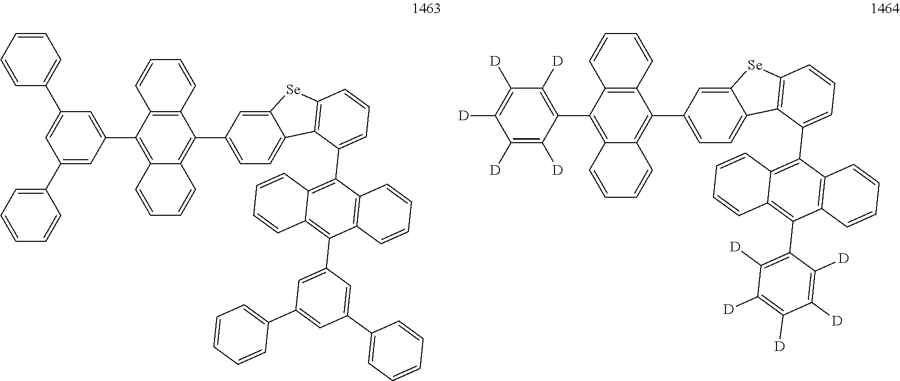 [SMILES: c1ccc(-c2cc(-c3ccccc3)cc(-c3c4ccccc4c(-c4ccc5c(c4)[se]c4cccc(-c6c7ccccc7c(-c7cc(-c8ccccc8)cc(-c8ccccc8)c7)c7ccccc67)c45)c4ccccc34)c2)cc1 ; [2H]c1c([2H])c([2H])c(-c2c3ccccc3c(-c3ccc4c(c3)[se]c3cccc(-c5c6ccccc6c(-c6c([2H])c([2H])c([2H])c([2H])c6[2H])c6ccccc56)c34)c3ccccc23)c([2H])c1[2H]]

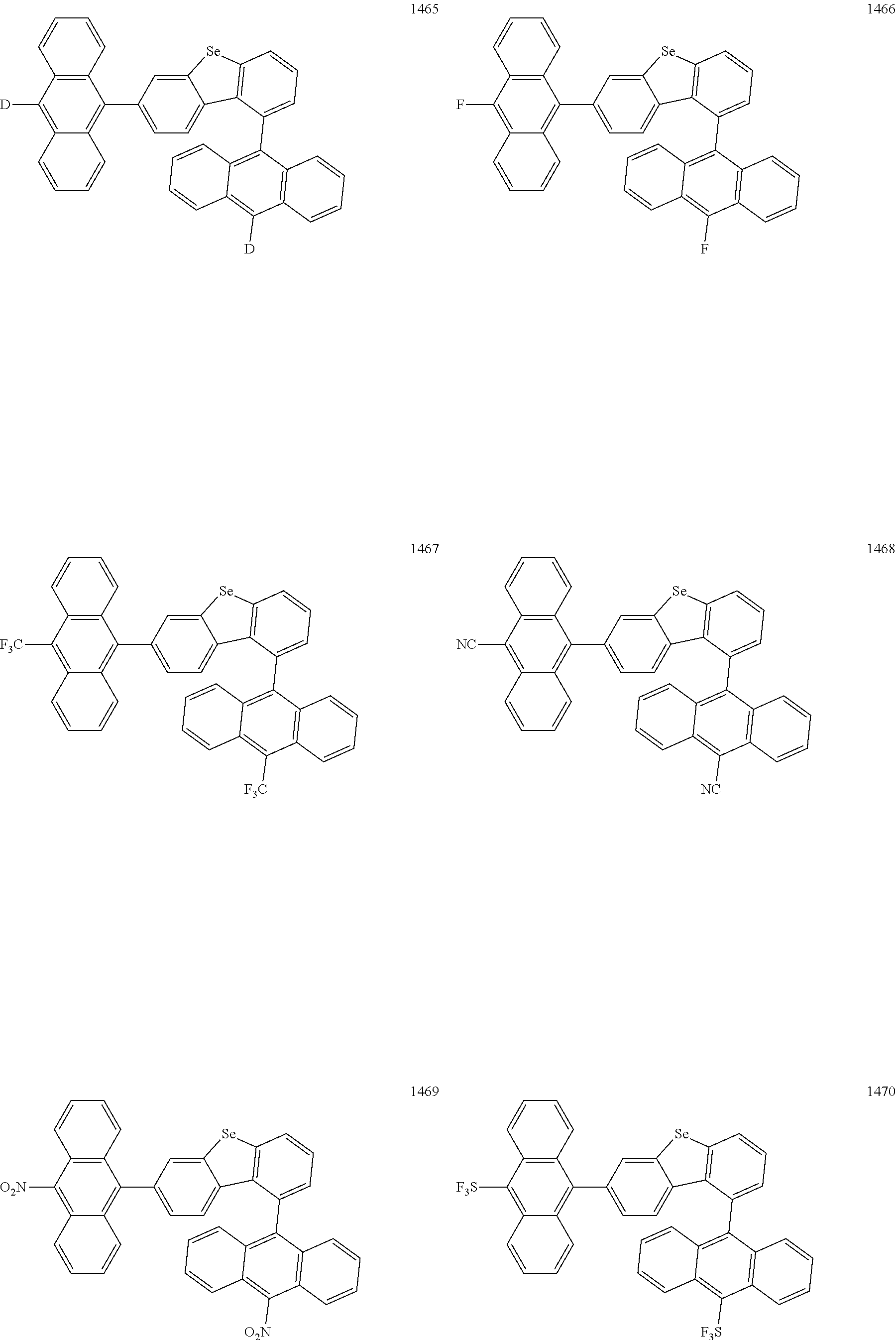

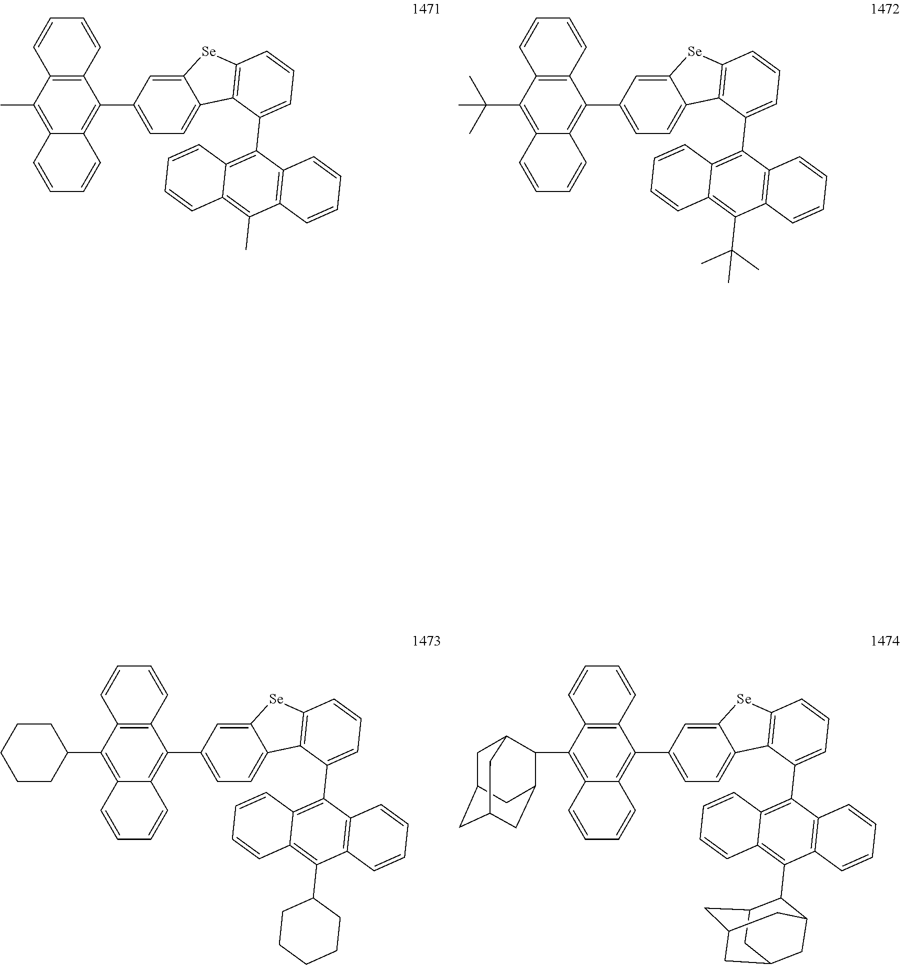 [SMILES: Cc1c2ccccc2c(-c2ccc3c(c2)[se]c2cccc(-c4c5ccccc5c(C)c5ccccc45)c23)c2ccccc12 ; CC(C)(C)c1c2ccccc2c(-c2ccc3c(c2)[se]c2cccc(-c4c5ccccc5c(C(C)(C)C)c5ccccc45)c23)c2ccccc12 ; c1cc(-c2c3ccccc3c(C3CCCCC3)c3ccccc23)c2c(c1)[se]c1cc(-c3c4ccccc4c(C4CCCCC4)c4ccccc34)ccc12 ; c1cc(-c2c3ccccc3c(C3C4CC5CC(C4)CC3C5)c3ccccc23)c2c(c1)[se]c1cc(-c3c4ccccc4c(C4C5CC6CC(C5)CC4C6)c4ccccc34)ccc12]

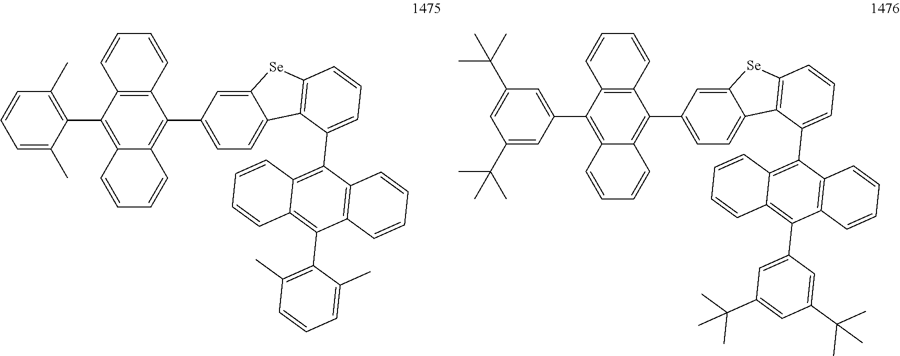 [SMILES: Cc1cccc(C)c1-c1c2ccccc2c(-c2ccc3c(c2)[se]c2cccc(-c4c5ccccc5c(-c5c(C)cccc5C)c5ccccc45)c23)c2ccccc12 ; CC(C)(C)c1cc(-c2c3ccccc3c(-c3ccc4c(c3)[se]c3cccc(-c5c6ccccc6c(-c6cc(C(C)(C)C)cc(C(C)(C)C)c6)c6ccccc56)c34)c3ccccc23)cc(C(C)(C)C)c1]

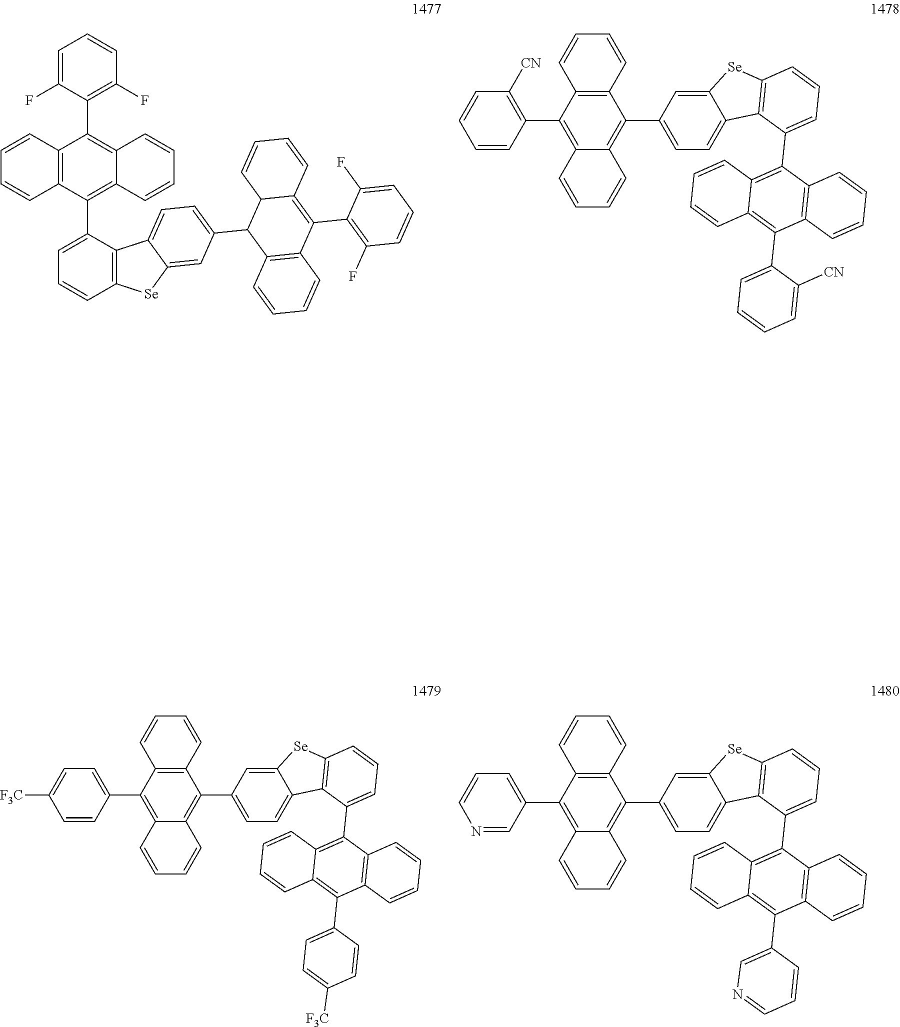 [SMILES: Fc1cccc(F)c1C1=C2C=CC=CC2C(c2ccc3c(c2)[se]c2cccc(-c4c5ccccc5c(-c5c(F)cccc5F)c5ccccc45)c23)c2ccccc21 ; N#Cc1ccccc1-c1c2ccccc2c(-c2ccc3c(c2)[se]c2cccc(-c4c5ccccc5c(-c5ccccc5C#N)c5ccccc45)c23)c2ccccc12 ; FC(F)(F)c1ccc(-c2c3ccccc3c(-c3ccc4c(c3)[se]c3cccc(-c5c6ccccc6c(-c6ccc(C(F)(F)F)cc6)c6ccccc56)c34)c3ccccc23)cc1 ; c1cncc(-c2c3ccccc3c(-c3ccc4c(c3)[se]c3cccc(-c5c6ccccc6c(-c6cccnc6)c6ccccc56)c34)c3ccccc23)c1]

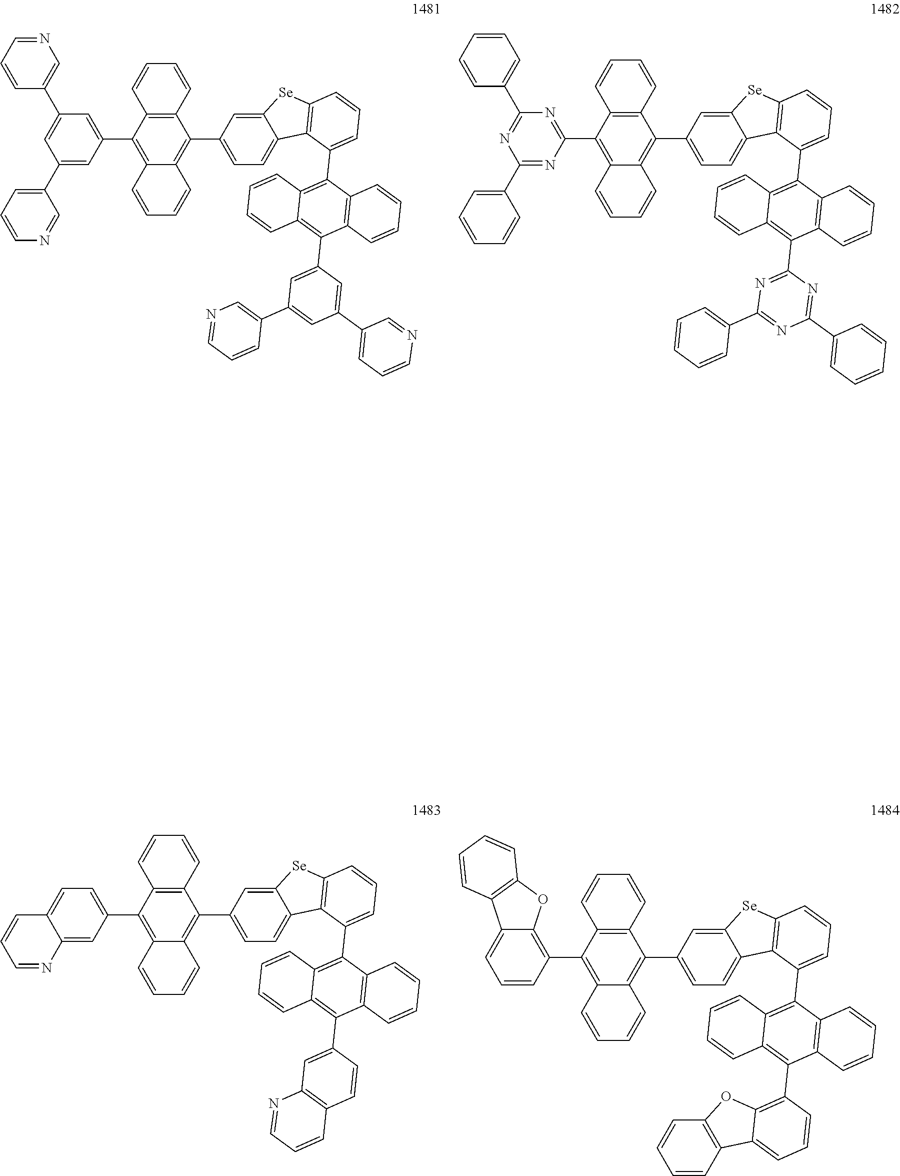

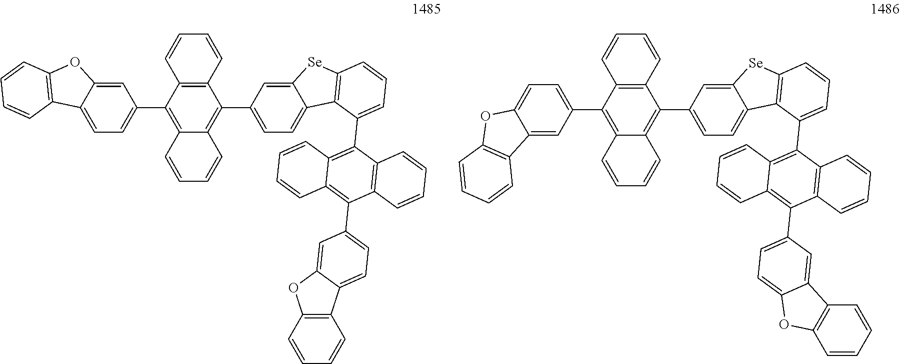

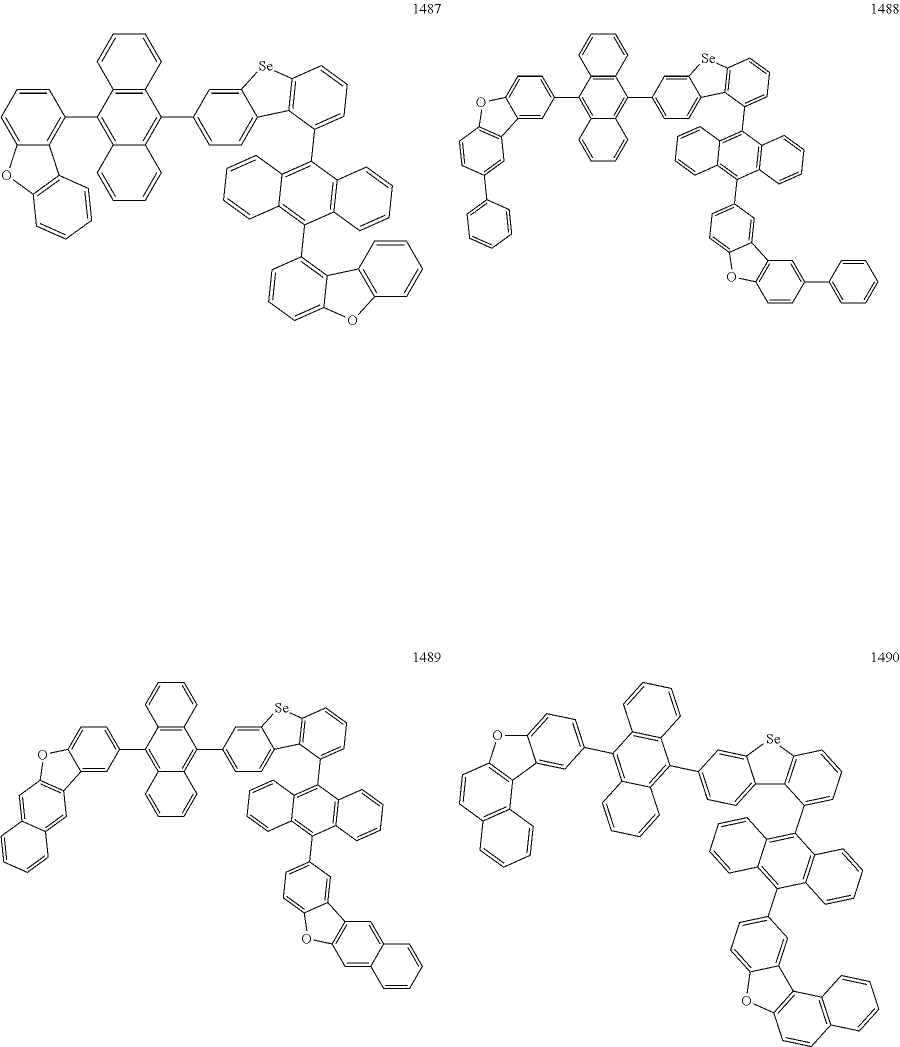

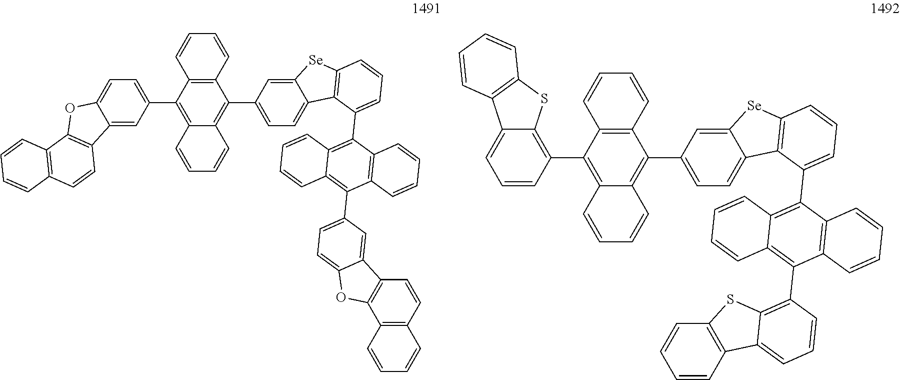 [SMILES: c1ccc2c(c1)ccc1c3cc(-c4c5ccccc5c(-c5ccc6c(c5)[se]c5cccc(-c7c8ccccc8c(-c8ccc9oc%10c%11ccccc%11ccc%10c9c8)c8ccccc78)c56)c5ccccc45)ccc3oc21 ; c1ccc2c(c1)sc1c(-c3c4ccccc4c(-c4ccc5c(c4)[se]c4cccc(-c6c7ccccc7c(-c7cccc8c7sc7ccccc78)c7ccccc67)c45)c4ccccc34)cccc12]

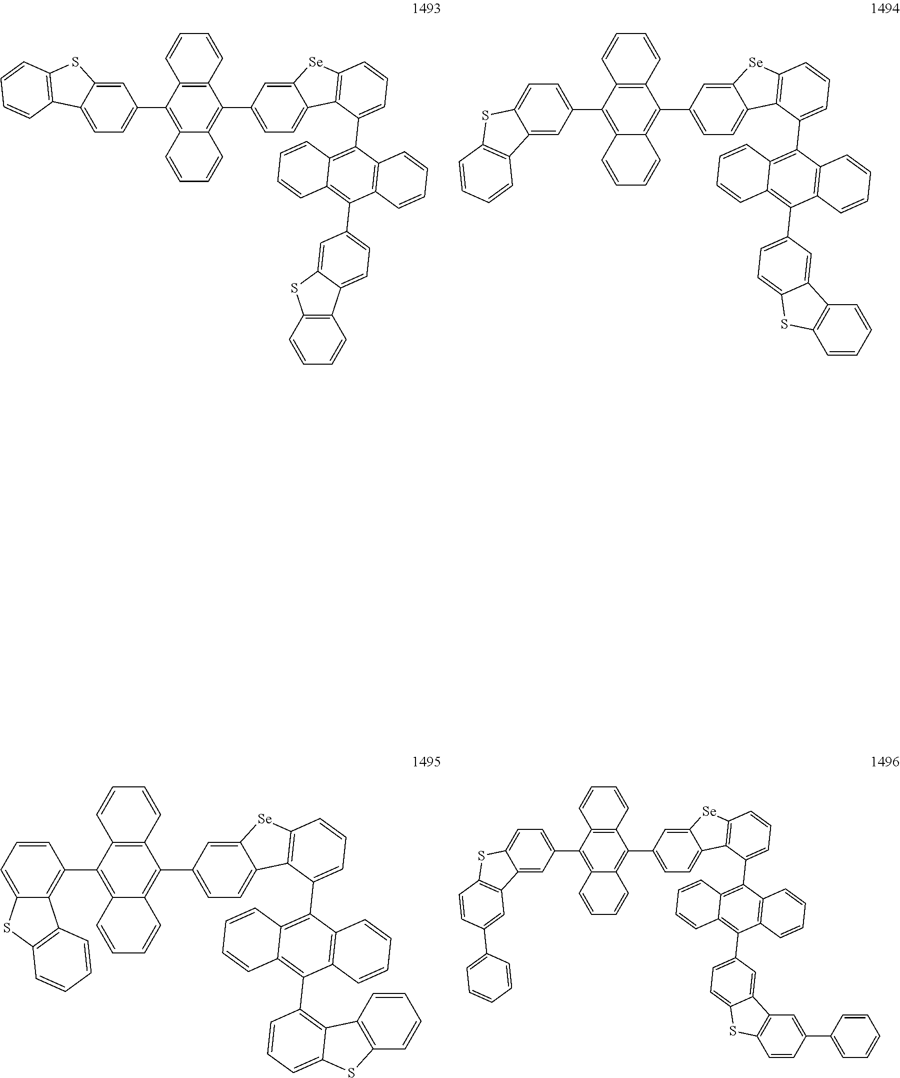

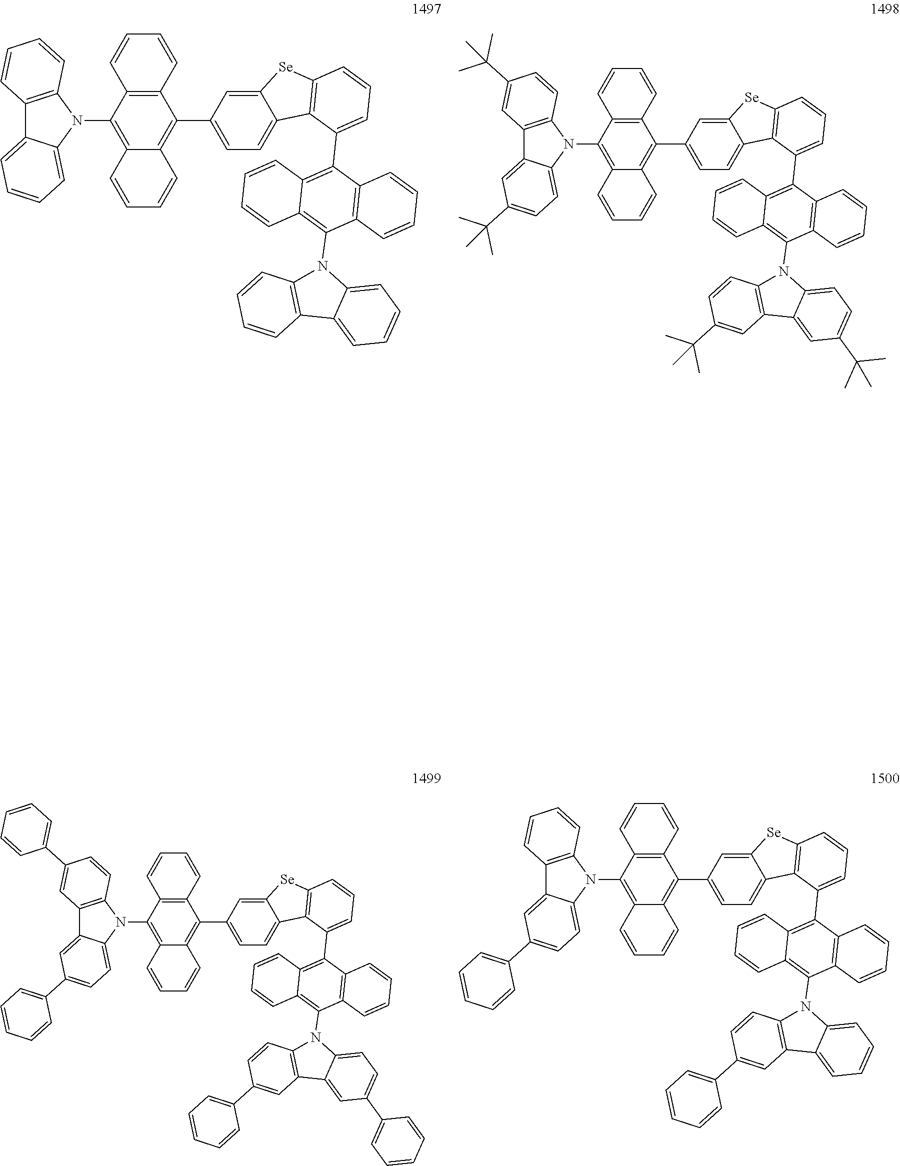

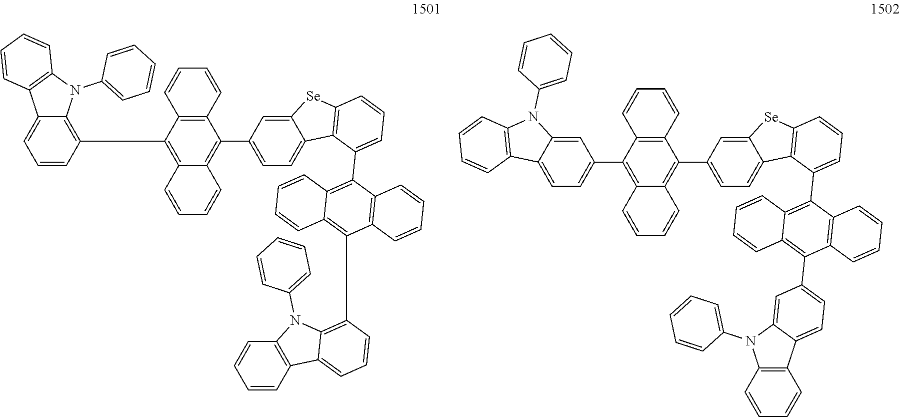 [SMILES: c1ccc(-n2c3ccccc3c3cccc(CCc4c5ccccc5c(-c5ccc6c(c5)[se]c5cccc(-c7c8ccccc8c(CCc8cccc9c%10ccccc%10n(-c%10ccccc%10)c89)c8ccccc78)c56)c5ccccc45)c32)cc1 ; c1ccc(-n2c3ccccc3c3ccc(-c4c5ccccc5c(-c5ccc6c(c5)[se]c5cccc(-c7c8ccccc8c(-c8ccc9c%10ccccc%10n(-c%10ccccc%10)c9c8)c8ccccc78)c56)c5ccccc45)cc32)cc1]

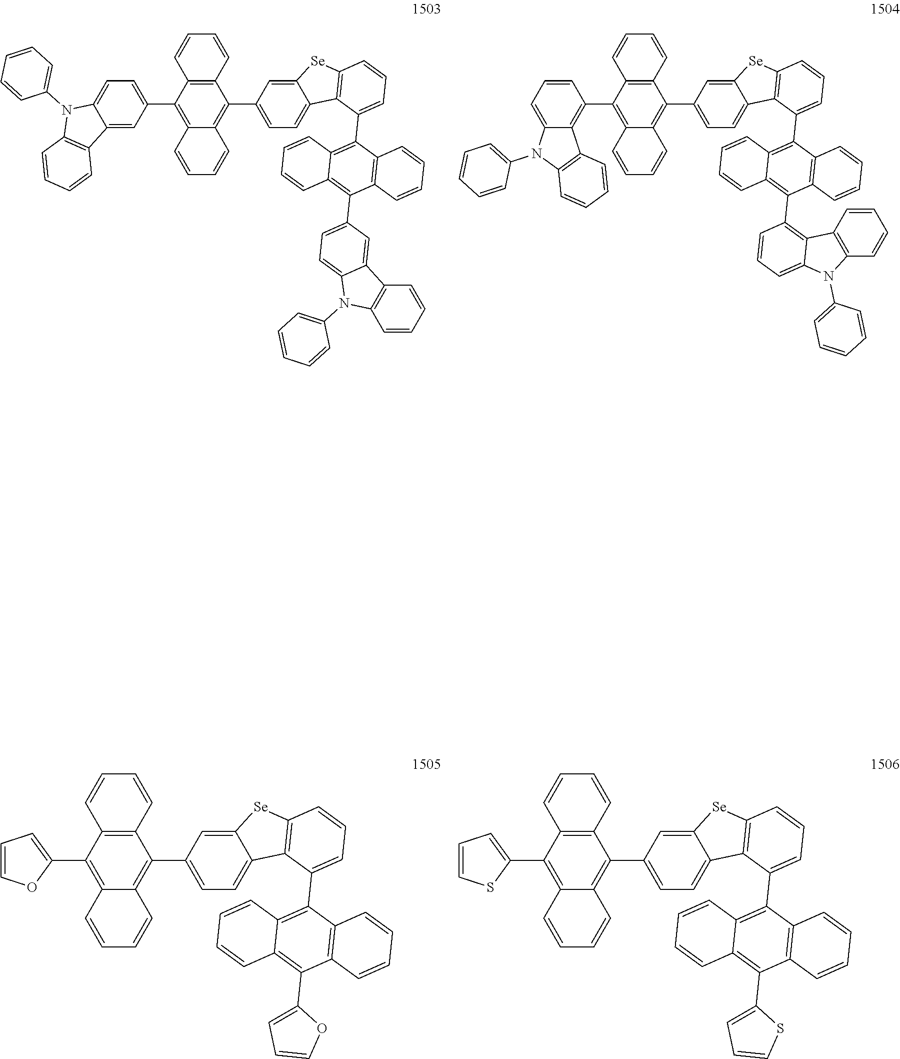 [SMILES: c1ccc(-n2c3ccccc3c3cc(-c4c5ccccc5c(-c5ccc6c(c5)[se]c5cccc(-c7c8ccccc8c(-c8ccc9c(c8)c8ccccc8n9-c8ccccc8)c8ccccc78)c56)c5ccccc45)ccc32)cc1 ; c1ccc(-n2c3ccccc3c3c(-c4c5ccccc5c(-c5ccc6c(c5)[se]c5cccc(-c7c8ccccc8c(-c8cccc9c8c8ccccc8n9-c8ccccc8)c8ccccc78)c56)c5ccccc45)cccc32)cc1 ; c1coc(-c2c3ccccc3c(-c3ccc4c(c3)[se]c3cccc(-c5c6ccccc6c(-c6ccco6)c6ccccc56)c34)c3ccccc23)c1 ; c1csc(-c2c3ccccc3c(-c3ccc4c(c3)[se]c3cccc(-c5c6ccccc6c(-c6cccs6)c6ccccc56)c34)c3ccccc23)c1]

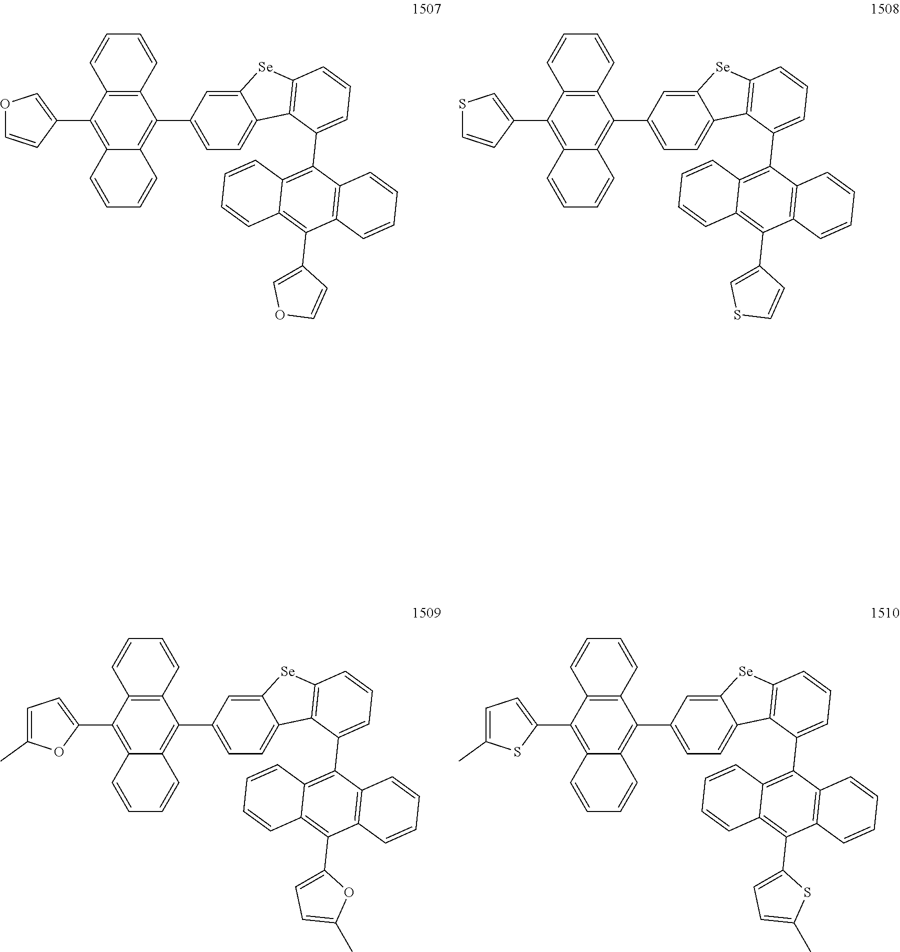 [SMILES: c1cc(-c2c3ccccc3c(-c3ccoc3)c3ccccc23)c2c(c1)[se]c1cc(-c3c4ccccc4c(-c4ccoc4)c4ccccc34)ccc12 ; c1cc(-c2c3ccccc3c(-c3ccsc3)c3ccccc23)c2c(c1)[se]c1cc(-c3c4ccccc4c(-c4ccsc4)c4ccccc34)ccc12 ; Cc1ccc(-c2c3ccccc3c(-c3ccc4c(c3)[se]c3cccc(-c5c6ccccc6c(-c6ccc(C)o6)c6ccccc56)c34)c3ccccc23)o1 ; Cc1ccc(-c2c3ccccc3c(-c3ccc4c(c3)[se]c3cccc(-c5c6ccccc6c(-c6ccc(C)s6)c6ccccc56)c34)c3ccccc23)s1]

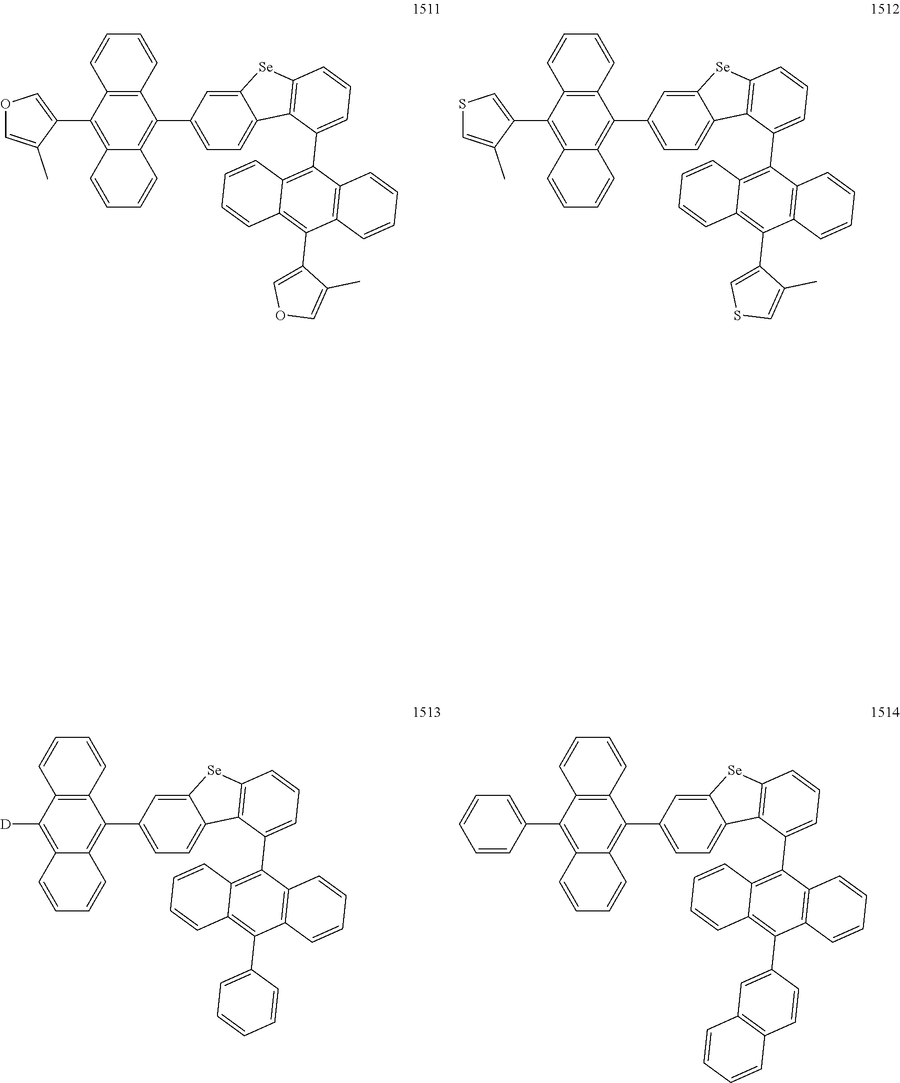 [SMILES: Cc1cocc1-c1c2ccccc2c(-c2ccc3c(c2)[se]c2cccc(-c4c5ccccc5c(-c5cocc5C)c5ccccc45)c23)c2ccccc12 ; Cc1cscc1-c1c2ccccc2c(-c2ccc3c(c2)[se]c2cccc(-c4c5ccccc5c(-c5cscc5C)c5ccccc45)c23)c2ccccc12 ; [2H]c1c2ccccc2c(-c2ccc3c(c2)[se]c2cccc(-c4c5ccccc5c(-c5ccccc5)c5ccccc45)c23)c2ccccc12 ; c1ccc(-c2c3ccccc3c(-c3ccc4c(c3)[se]c3cccc(-c5c6ccccc6c(-c6ccc7ccccc7c6)c6ccccc56)c34)c3ccccc23)cc1]

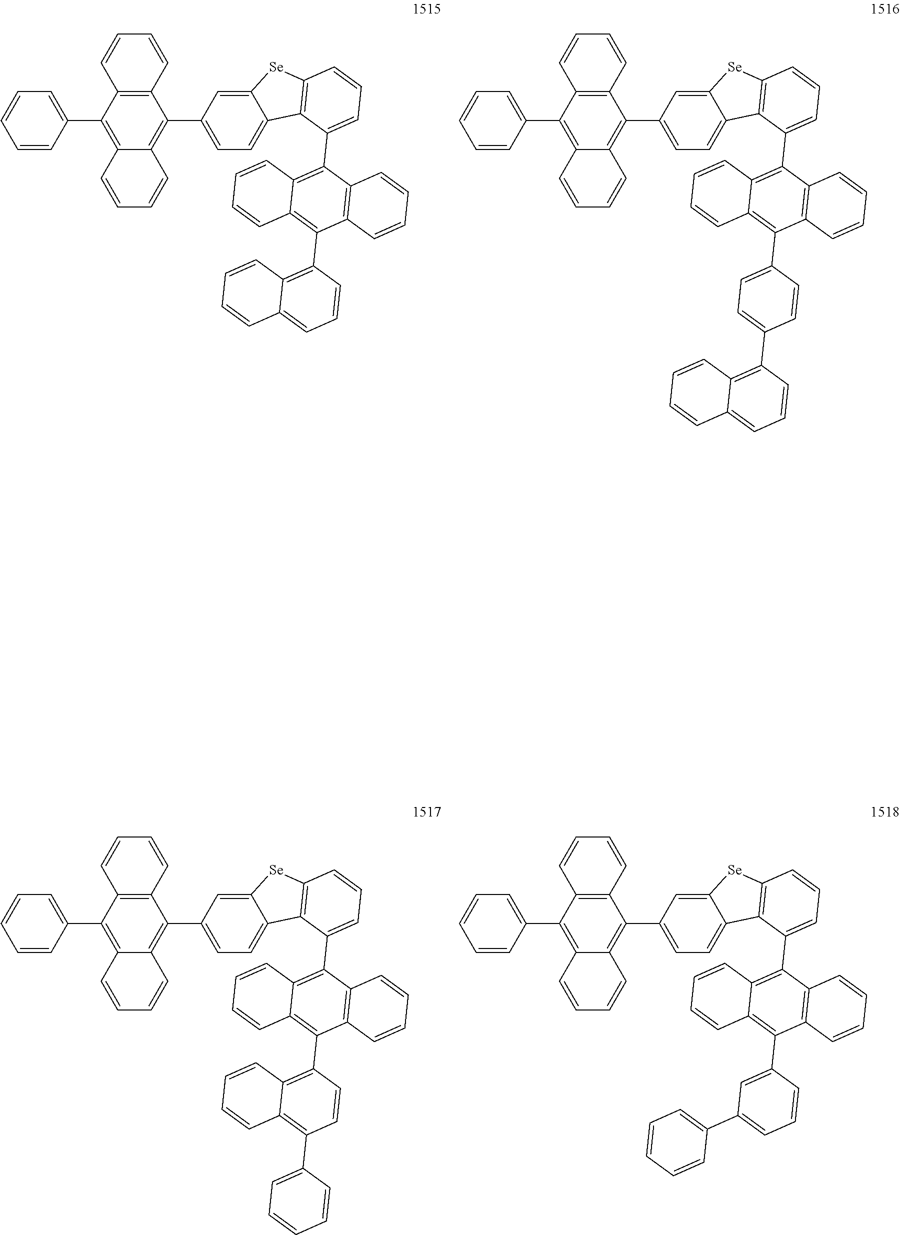 [SMILES: c1ccc(-c2c3ccccc3c(-c3ccc4c(c3)[se]c3cccc(-c5c6ccccc6c(-c6cccc7ccccc67)c6ccccc56)c34)c3ccccc23)cc1 ; c1ccc(-c2c3ccccc3c(-c3ccc4c(c3)[se]c3cccc(-c5c6ccccc6c(-c6ccc(-c7cccc8ccccc78)cc6)c6ccccc56)c34)c3ccccc23)cc1 ; c1ccc(-c2ccc(-c3c4ccccc4c(-c4cccc5[se]c6cc(-c7c8ccccc8c(-c8ccccc8)c8ccccc78)ccc6c45)c4ccccc34)c3ccccc23)cc1 ; c1ccc(-c2cccc(-c3c4ccccc4c(-c4cccc5[se]c6cc(-c7c8ccccc8c(-c8ccccc8)c8ccccc78)ccc6c45)c4ccccc34)c2)cc1]

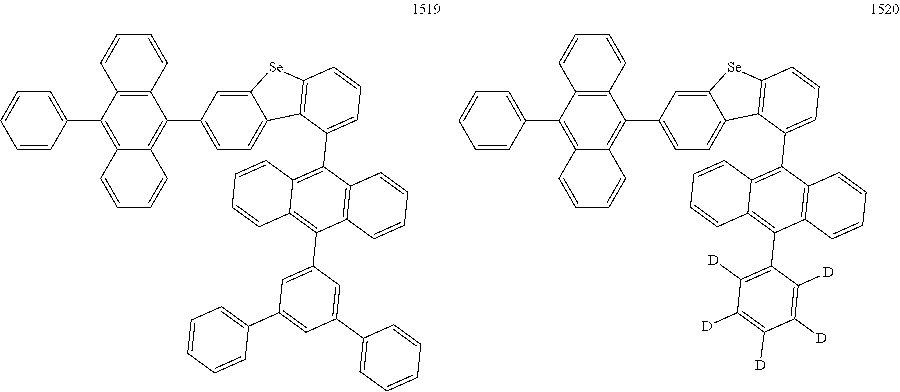 [SMILES: c1ccc(-c2cc(-c3ccccc3)cc(-c3c4ccccc4c(-c4cccc5[se]c6cc(-c7c8ccccc8c(-c8ccccc8)c8ccccc78)ccc6c45)c4ccccc34)c2)cc1 ; [2H]c1c([2H])c([2H])c(-c2c3ccccc3c(-c3cccc4[se]c5cc(-c6c7ccccc7c(-c7ccccc7)c7ccccc67)ccc5c34)c3ccccc23)c([2H])c1[2H]]

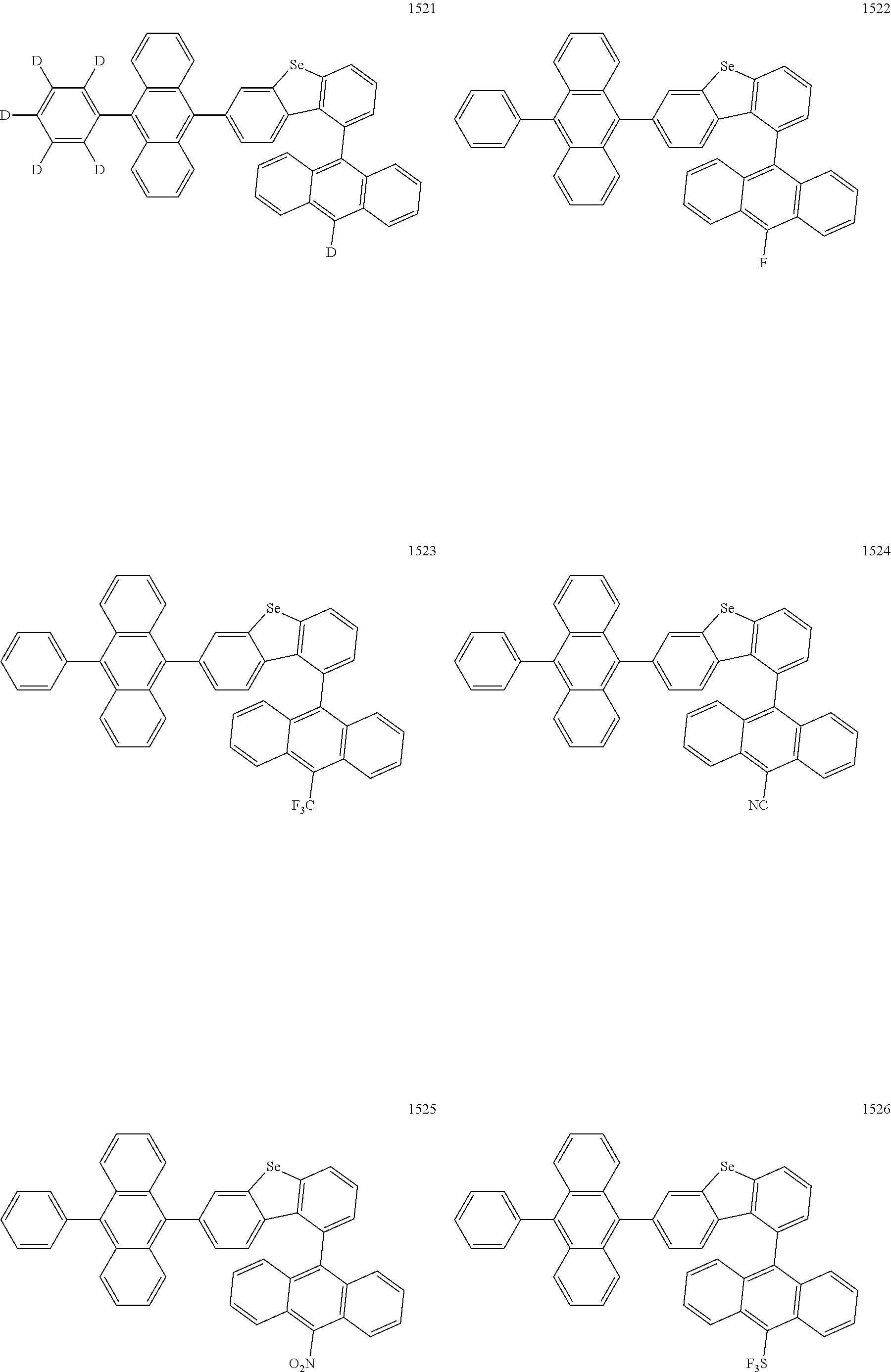

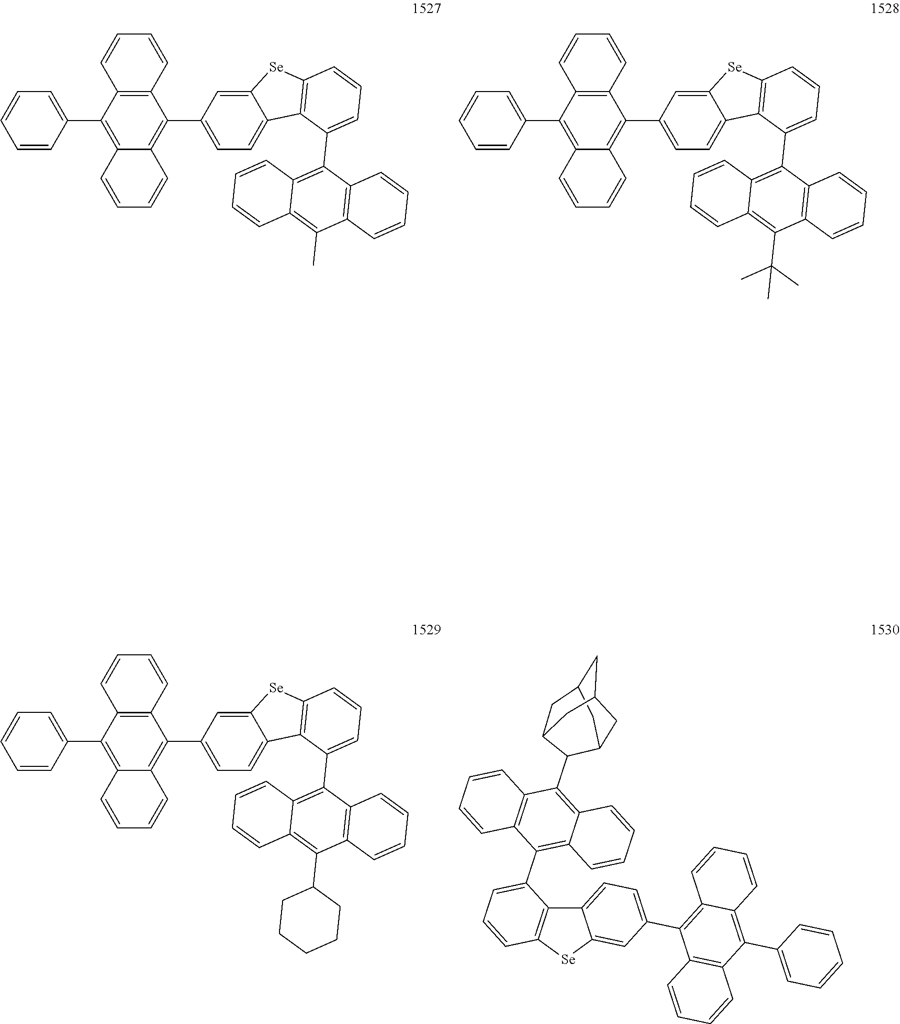 [SMILES: Cc1c2ccccc2c(-c2cccc3[se]c4cc(-c5c6ccccc6c(-c6ccccc6)c6ccccc56)ccc4c23)c2ccccc12 ; CC(C)(C)c1c2ccccc2c(-c2cccc3[se]c4cc(-c5c6ccccc6c(-c6ccccc6)c6ccccc56)ccc4c23)c2ccccc12 ; c1ccc(-c2c3ccccc3c(-c3ccc4c(c3)[se]c3cccc(-c5c6ccccc6c(C6CCCCC6)c6ccccc56)c34)c3ccccc23)cc1 ; c1ccc(-c2c3ccccc3c(-c3ccc4c(c3)[se]c3cccc(-c5c6ccccc6c(C6C7CC8CC(C7)CC6C8)c6ccccc56)c34)c3ccccc23)cc1]

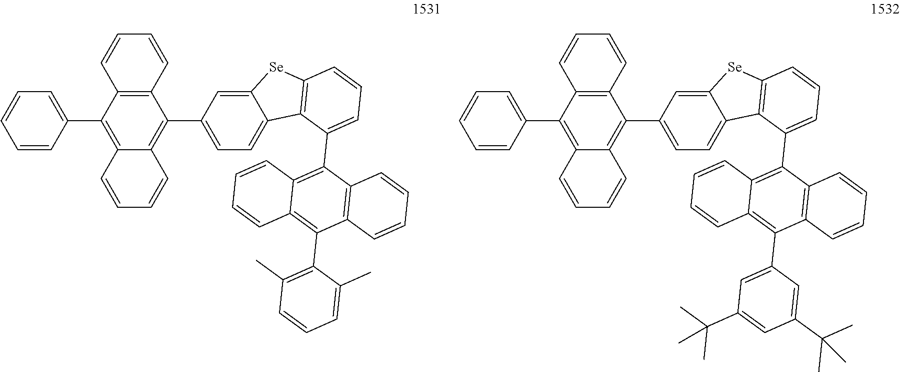 [SMILES: Cc1cccc(C)c1-c1c2ccccc2c(-c2cccc3[se]c4cc(-c5c6ccccc6c(-c6ccccc6)c6ccccc56)ccc4c23)c2ccccc12 ; CC(C)(C)c1cc(-c2c3ccccc3c(-c3cccc4[se]c5cc(-c6c7ccccc7c(-c7ccccc7)c7ccccc67)ccc5c34)c3ccccc23)cc(C(C)(C)C)c1]

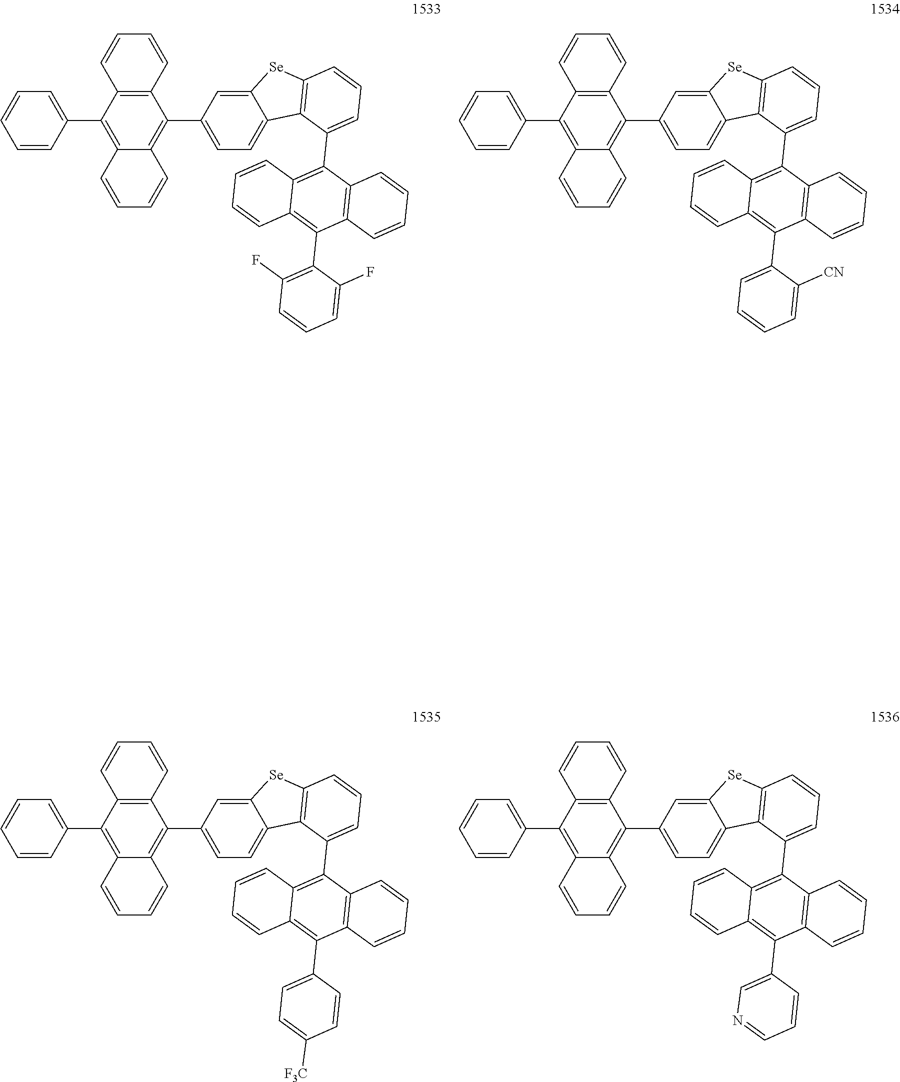 [SMILES: Fc1cccc(F)c1-c1c2ccccc2c(-c2cccc3[se]c4cc(-c5c6ccccc6c(-c6ccccc6)c6ccccc56)ccc4c23)c2ccccc12 ; N#Cc1ccccc1-c1c2ccccc2c(-c2cccc3[se]c4cc(-c5c6ccccc6c(-c6ccccc6)c6ccccc56)ccc4c23)c2ccccc12 ; FC(F)(F)c1ccc(-c2c3ccccc3c(-c3cccc4[se]c5cc(-c6c7ccccc7c(-c7ccccc7)c7ccccc67)ccc5c34)c3ccccc23)cc1 ; c1ccc(-c2c3ccccc3c(-c3ccc4c(c3)[se]c3cccc(-c5c6ccccc6c(-c6cccnc6)c6ccccc56)c34)c3ccccc23)cc1]

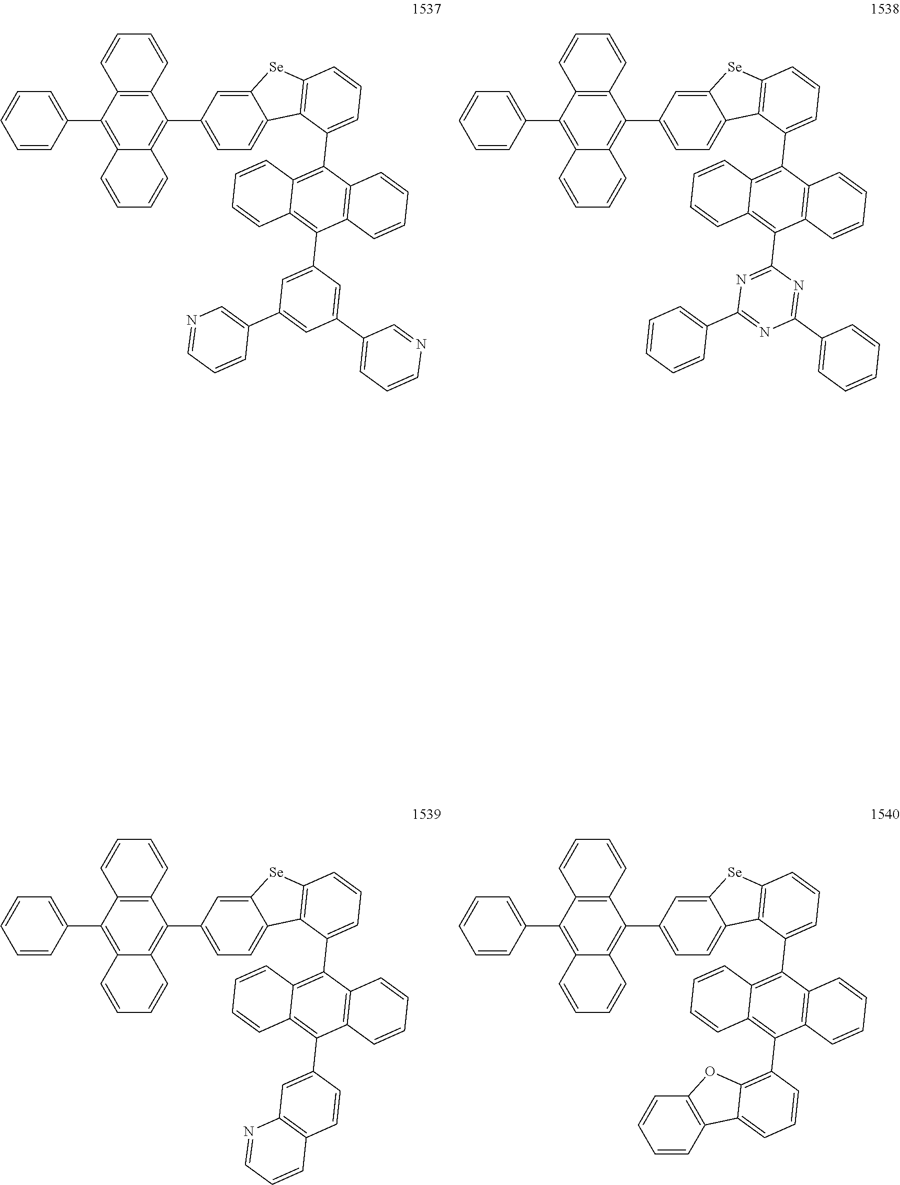 [SMILES: c1ccc(-c2c3ccccc3c(-c3ccc4c(c3)[se]c3cccc(-c5c6ccccc6c(-c6cc(-c7cccnc7)cc(-c7cccnc7)c6)c6ccccc56)c34)c3ccccc23)cc1 ; c1ccc(-c2nc(-c3ccccc3)nc(-c3c4ccccc4c(-c4cccc5[se]c6cc(-c7c8ccccc8c(-c8ccccc8)c8ccccc78)ccc6c45)c4ccccc34)n2)cc1 ; c1ccc(-c2c3ccccc3c(-c3ccc4c(c3)[se]c3cccc(-c5c6ccccc6c(-c6ccc7cccnc7c6)c6ccccc56)c34)c3ccccc23)cc1 ; c1ccc(-c2c3ccccc3c(-c3ccc4c(c3)[se]c3cccc(-c5c6ccccc6c(-c6cccc7c6oc6ccccc67)c6ccccc56)c34)c3ccccc23)cc1]

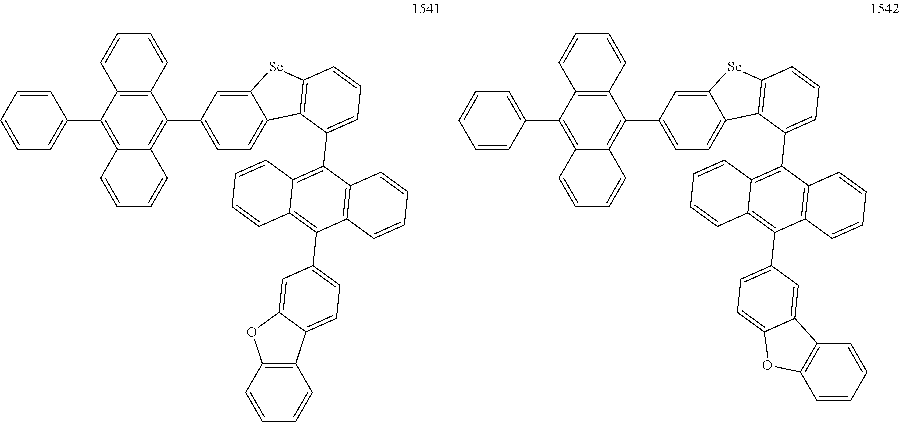 [SMILES: c1ccc(-c2c3ccccc3c(-c3ccc4c(c3)[se]c3cccc(-c5c6ccccc6c(-c6ccc7c(c6)oc6ccccc67)c6ccccc56)c34)c3ccccc23)cc1 ; c1ccc(-c2c3ccccc3c(-c3ccc4c(c3)[se]c3cccc(-c5c6ccccc6c(-c6ccc7oc8ccccc8c7c6)c6ccccc56)c34)c3ccccc23)cc1]

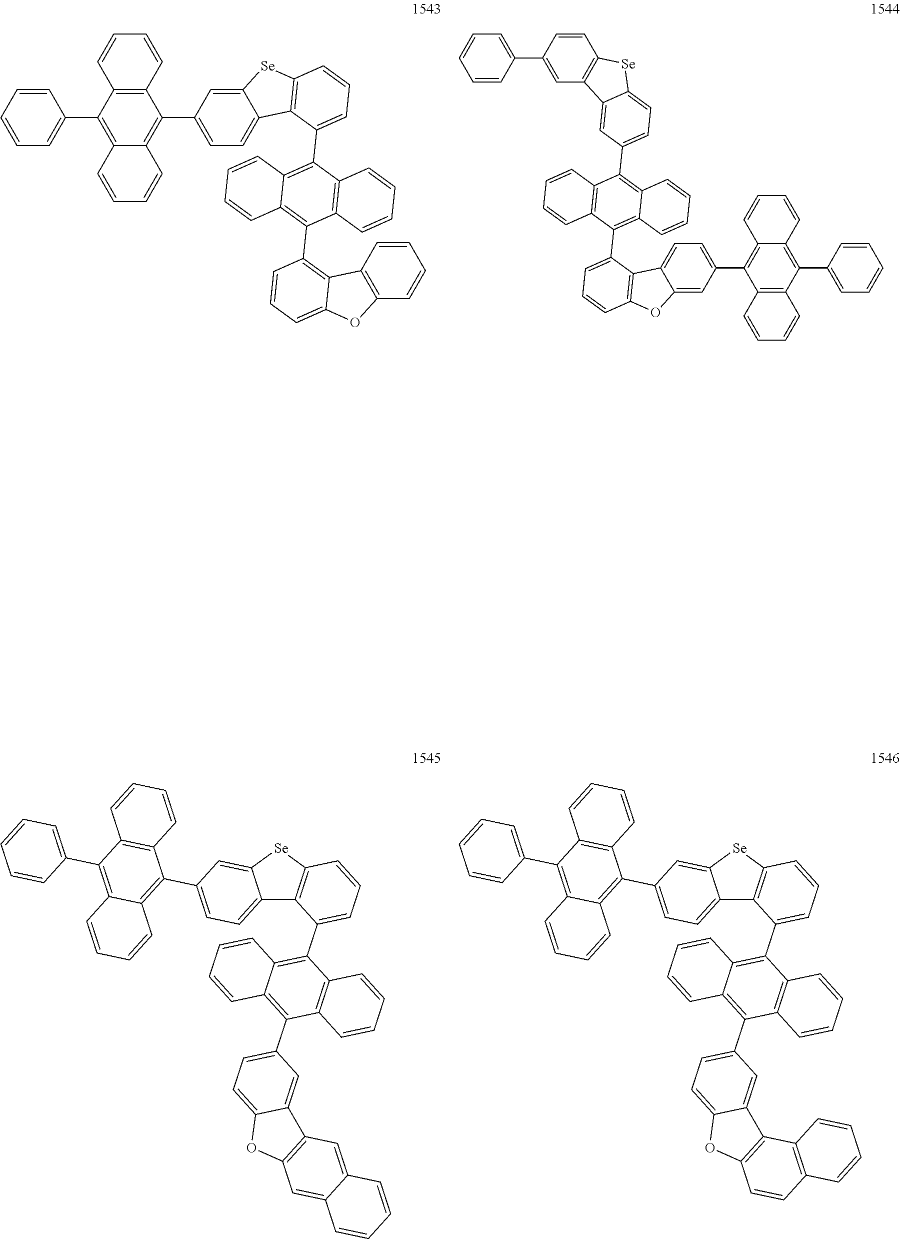 [SMILES: c1ccc(-c2c3ccccc3c(-c3ccc4c(c3)[se]c3cccc(-c5c6ccccc6c(-c6cccc7oc8ccccc8c67)c6ccccc56)c34)c3ccccc23)cc1 ; c1ccc(-c2ccc3[se]c4ccc(-c5c6ccccc6c(-c6cccc7oc8cc(-c9c%10ccccc%10c(-c%10ccccc%10)c%10ccccc9%10)ccc8c67)c6ccccc56)cc4c3c2)cc1 ; c1ccc(-c2c3ccccc3c(-c3ccc4c(c3)[se]c3cccc(-c5c6ccccc6c(-c6ccc7oc8cc9ccccc9cc8c7c6)c6ccccc56)c34)c3ccccc23)cc1 ; c1ccc(-c2c3ccccc3c(-c3ccc4c(c3)[se]c3cccc(-c5c6ccccc6c(-c6ccc7oc8ccc9ccccc9c8c7c6)c6ccccc56)c34)c3ccccc23)cc1]

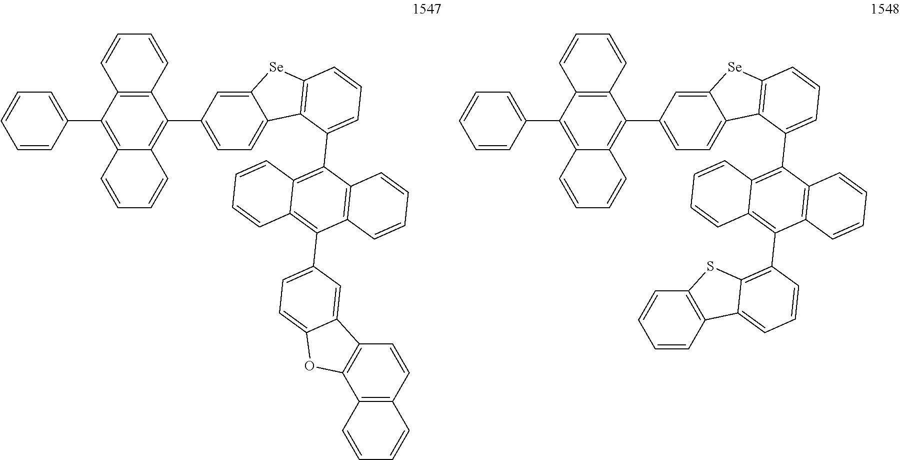 [SMILES: c1ccc(-c2c3ccccc3c(-c3ccc4c(c3)[se]c3cccc(-c5c6ccccc6c(-c6ccc7oc8c9ccccc9ccc8c7c6)c6ccccc56)c34)c3ccccc23)cc1 ; c1ccc(-c2c3ccccc3c(-c3ccc4c(c3)[se]c3cccc(-c5c6ccccc6c(-c6cccc7c6sc6ccccc67)c6ccccc56)c34)c3ccccc23)cc1]

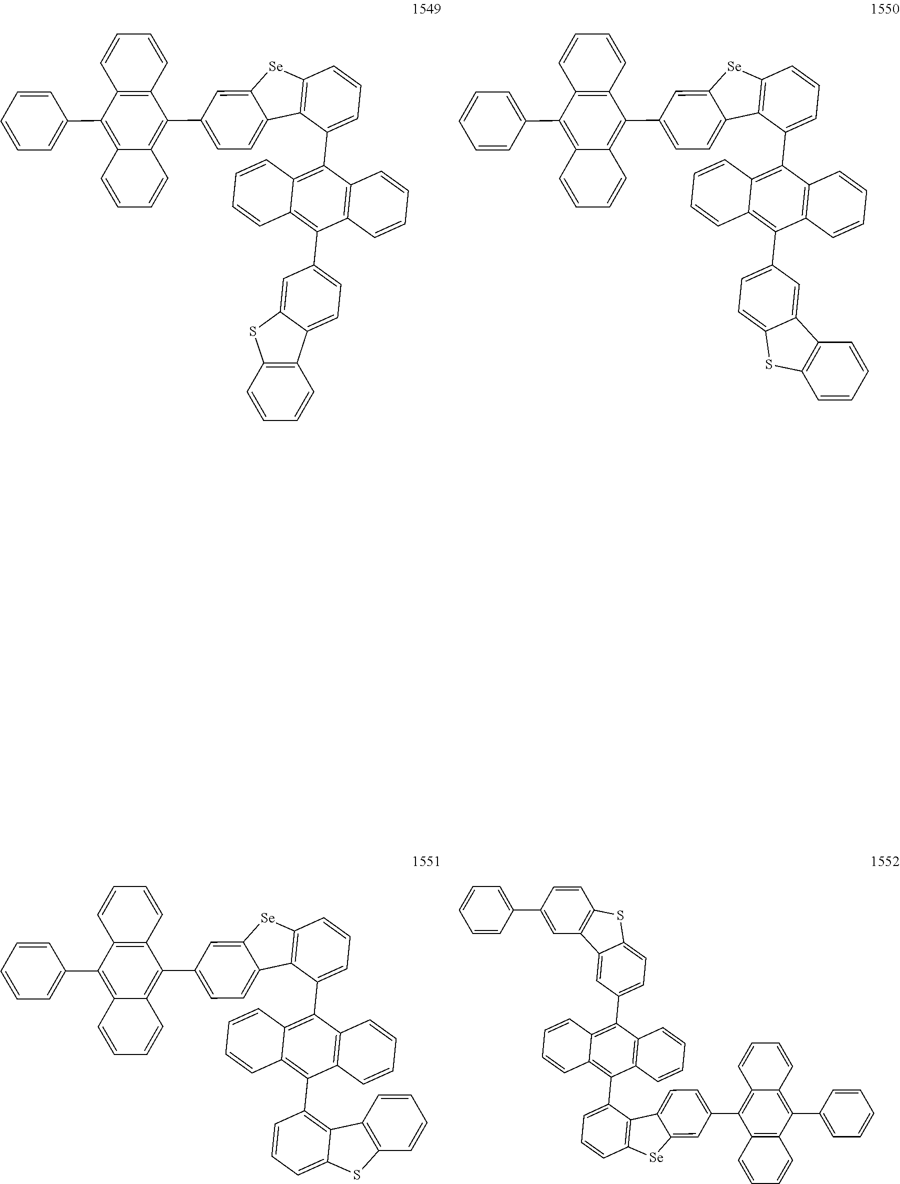 [SMILES: c1ccc(-c2c3ccccc3c(-c3ccc4c(c3)[se]c3cccc(-c5c6ccccc6c(-c6ccc7c(c6)sc6ccccc67)c6ccccc56)c34)c3ccccc23)cc1 ; c1ccc(-c2c3ccccc3c(-c3ccc4c(c3)[se]c3cccc(-c5c6ccccc6c(-c6ccc7sc8ccccc8c7c6)c6ccccc56)c34)c3ccccc23)cc1 ; c1ccc(-c2c3ccccc3c(-c3ccc4c(c3)[se]c3cccc(-c5c6ccccc6c(-c6cccc7sc8ccccc8c67)c6ccccc56)c34)c3ccccc23)cc1 ; c1ccc(-c2ccc3sc4ccc(-c5c6ccccc6c(-c6cccc7[se]c8cc(-c9c%10ccccc%10c(-c%10ccccc%10)c%10ccccc9%10)ccc8c67)c6ccccc56)cc4c3c2)cc1]

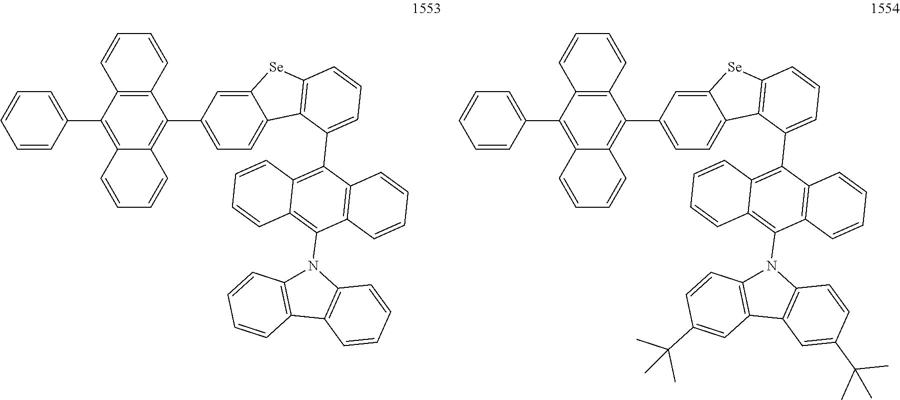 [SMILES: c1ccc(-c2c3ccccc3c(-c3ccc4c(c3)[se]c3cccc(-c5c6ccccc6c(-n6c7ccccc7c7ccccc76)c6ccccc56)c34)c3ccccc23)cc1 ; CC(C)(C)c1ccc2c(c1)c1cc(C(C)(C)C)ccc1n2-c1c2ccccc2c(-c2cccc3[se]c4cc(-c5c6ccccc6c(-c6ccccc6)c6ccccc56)ccc4c23)c2ccccc12]

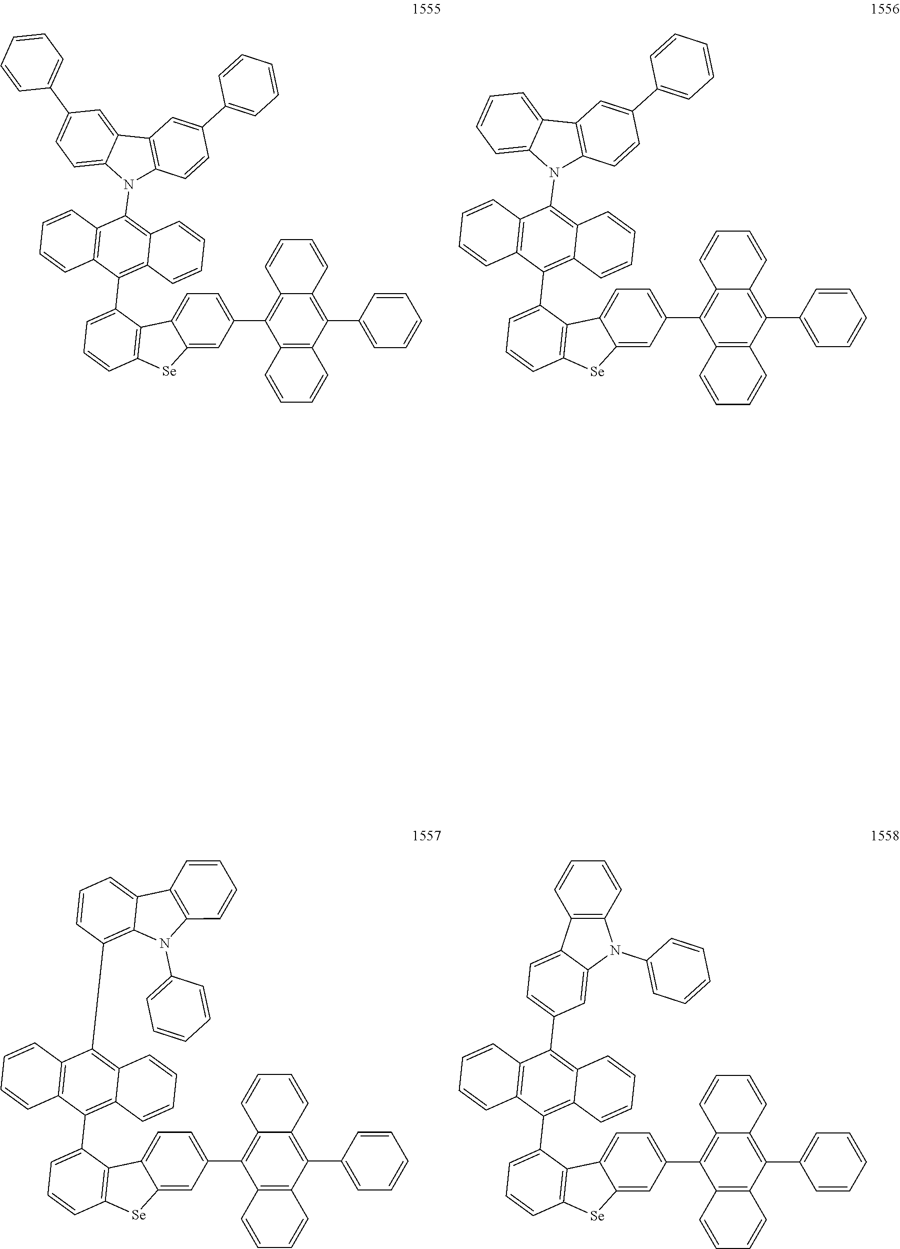 [SMILES: c1ccc(-c2ccc3c(c2)c2cc(-c4ccccc4)ccc2n3-c2c3ccccc3c(-c3cccc4[se]c5cc(-c6c7ccccc7c(-c7ccccc7)c7ccccc67)ccc5c34)c3ccccc23)cc1 ; c1ccc(-c2ccc3c(c2)c2ccccc2n3-c2c3ccccc3c(-c3cccc4[se]c5cc(-c6c7ccccc7c(-c7ccccc7)c7ccccc67)ccc5c34)c3ccccc23)cc1 ; c1ccc(-c2c3ccccc3c(-c3ccc4c(c3)[se]c3cccc(-c5c6ccccc6c(-c6cccc7c8ccccc8n(-c8ccccc8)c67)c6ccccc56)c34)c3ccccc23)cc1 ; c1ccc(-c2c3ccccc3c(-c3ccc4c(c3)[se]c3cccc(-c5c6ccccc6c(-c6ccc7c8ccccc8n(-c8ccccc8)c7c6)c6ccccc56)c34)c3ccccc23)cc1]

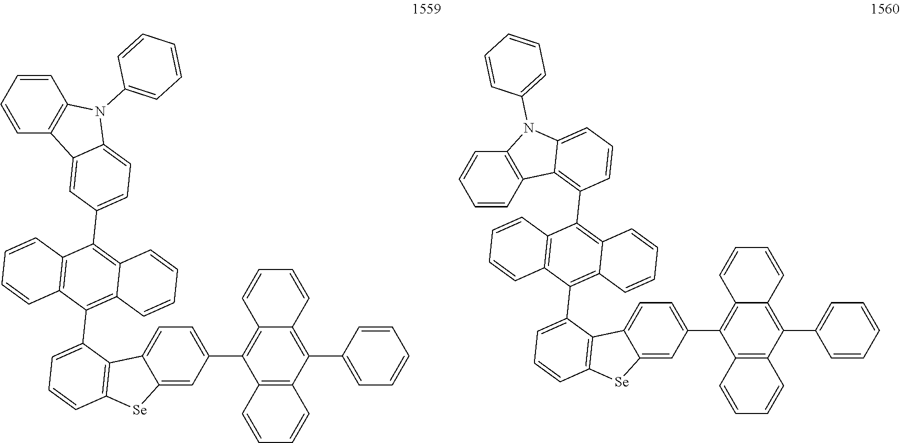 [SMILES: c1ccc(-c2c3ccccc3c(-c3ccc4c(c3)[se]c3cccc(-c5c6ccccc6c(-c6ccc7c(c6)c6ccccc6n7-c6ccccc6)c6ccccc56)c34)c3ccccc23)cc1 ; c1ccc(-c2c3ccccc3c(-c3ccc4c(c3)[se]c3cccc(-c5c6ccccc6c(-c6cccc7c6c6ccccc6n7-c6ccccc6)c6ccccc56)c34)c3ccccc23)cc1]

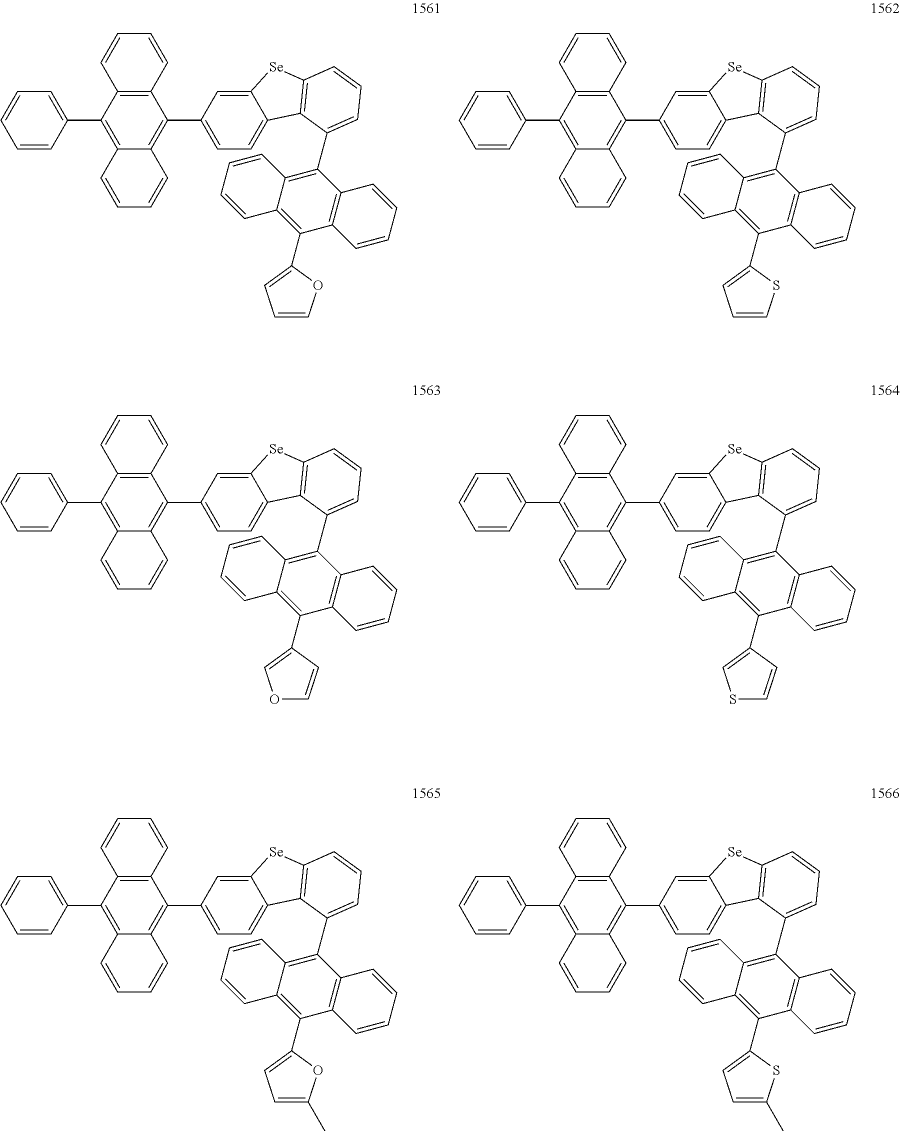

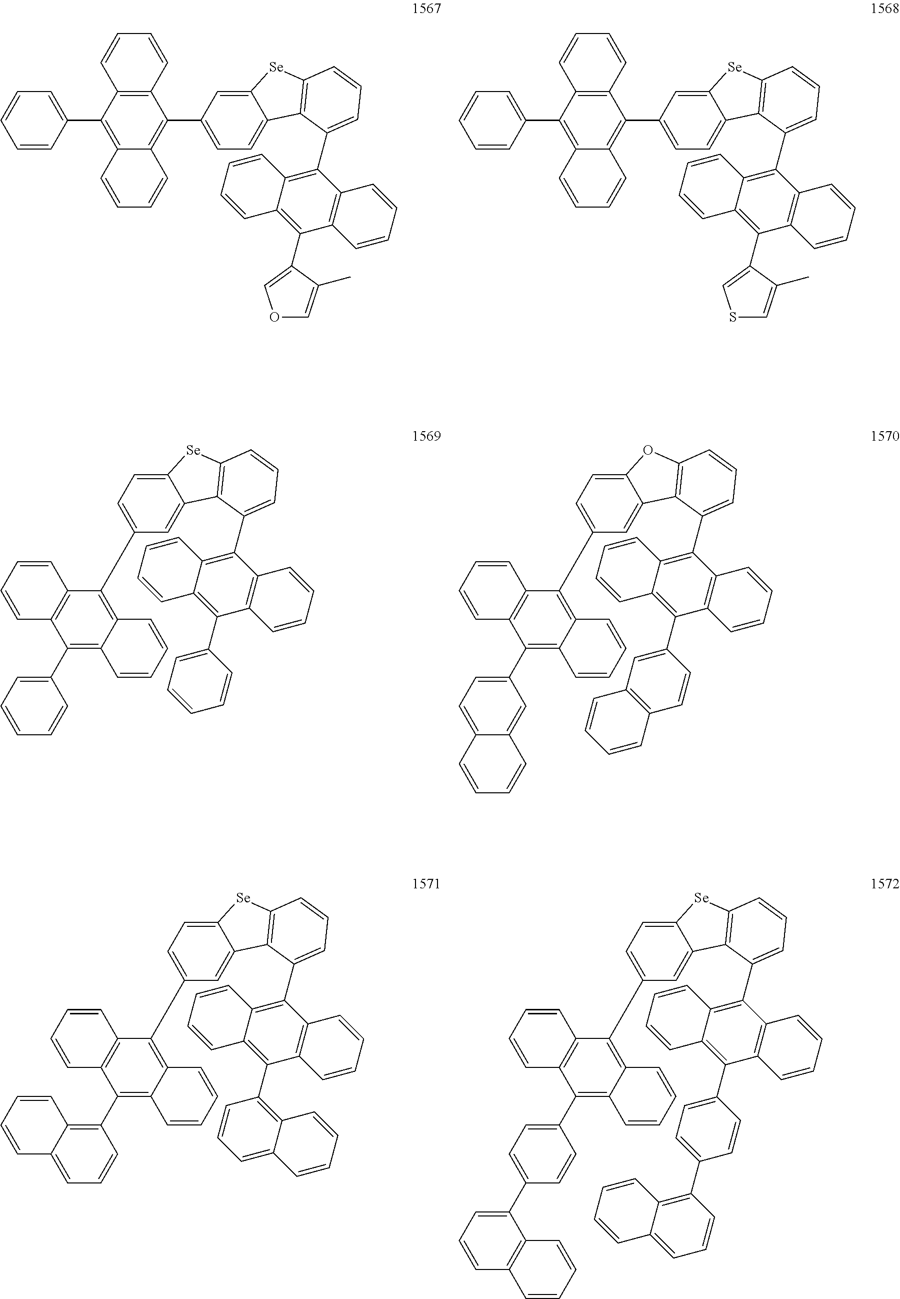

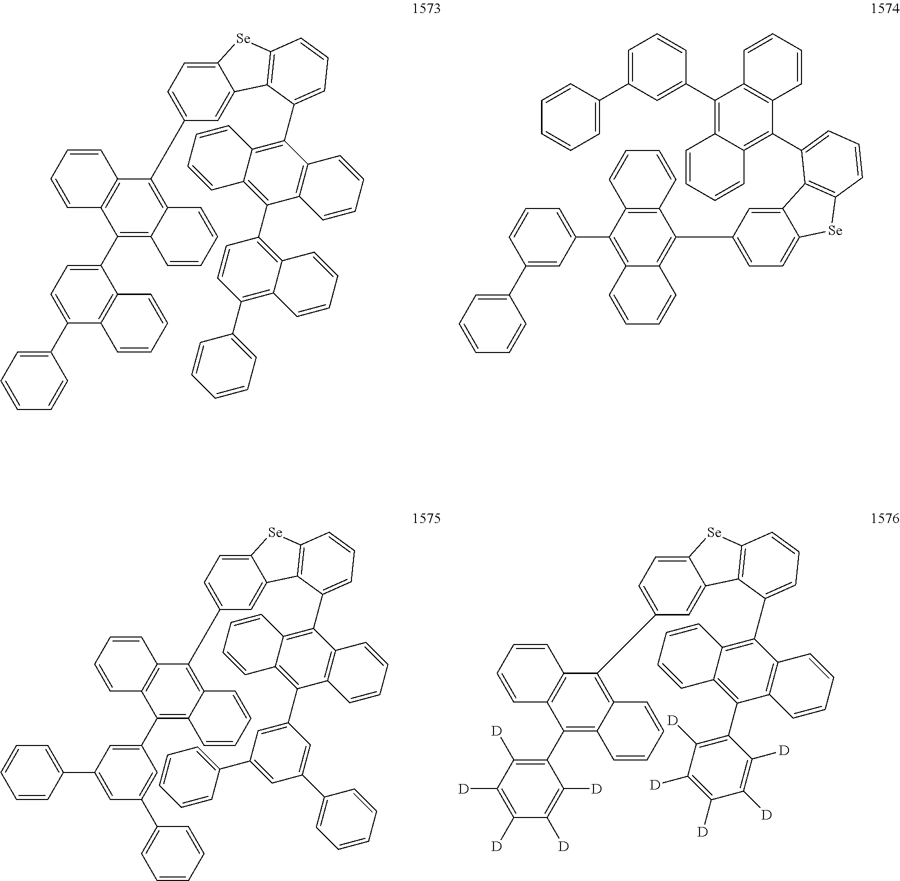

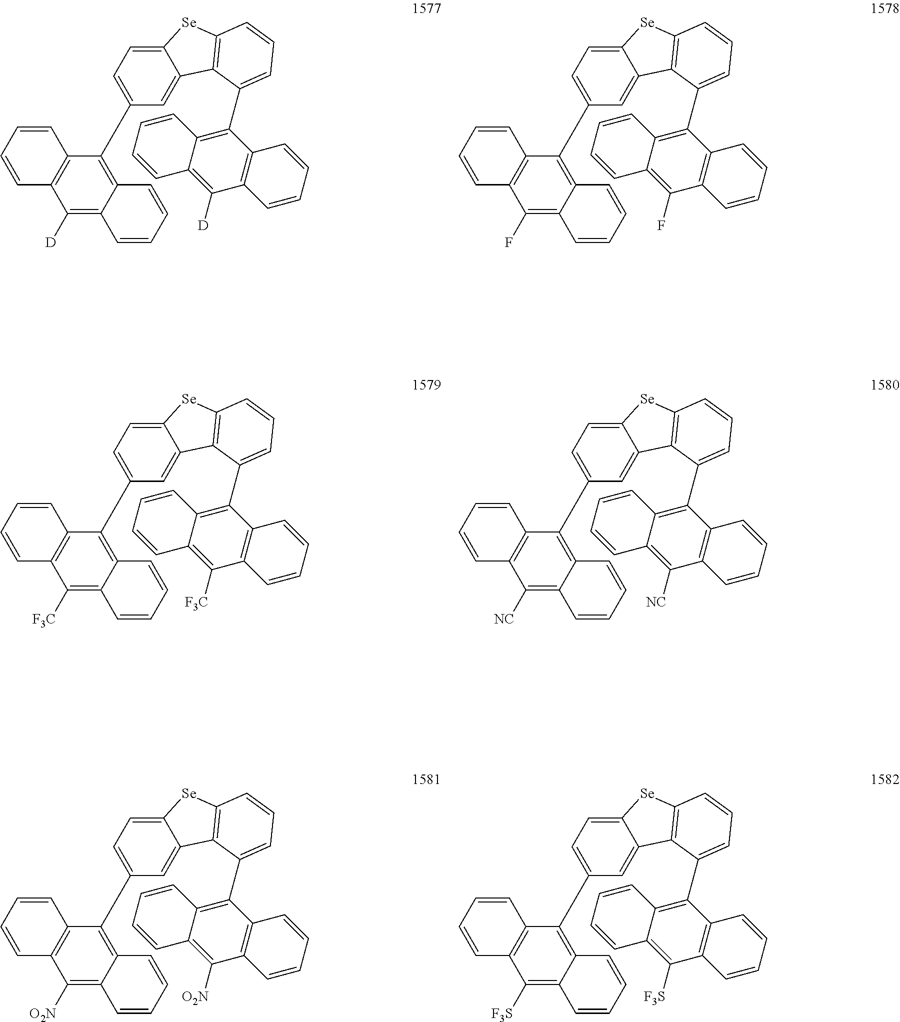

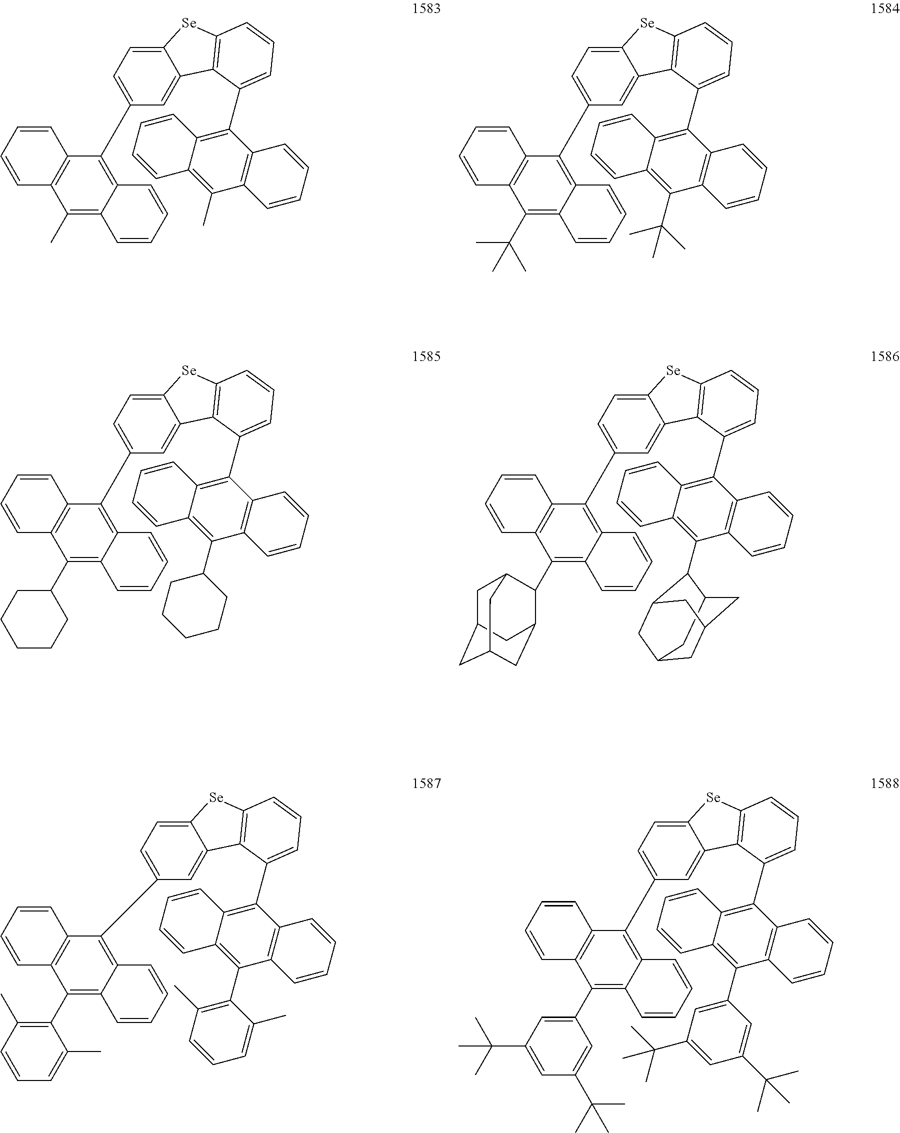

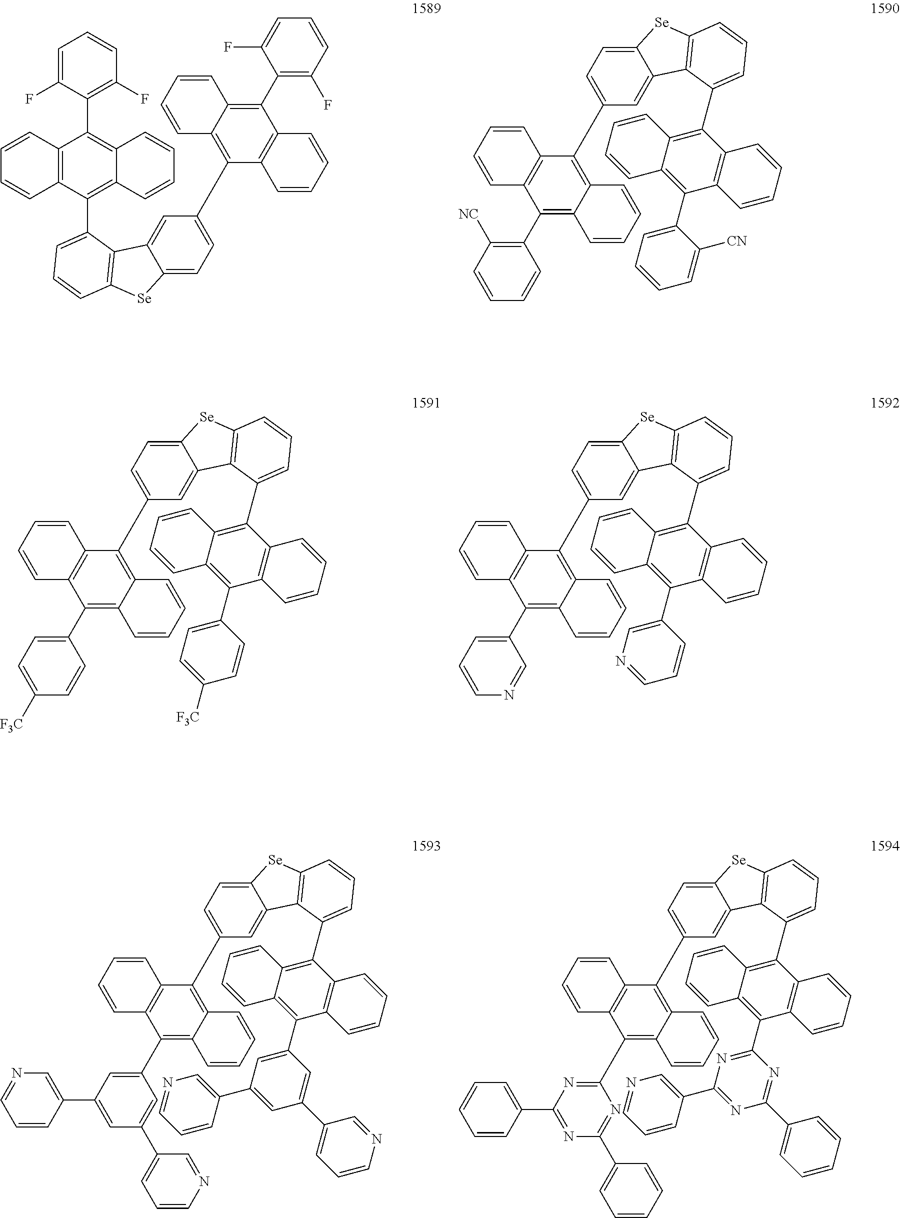

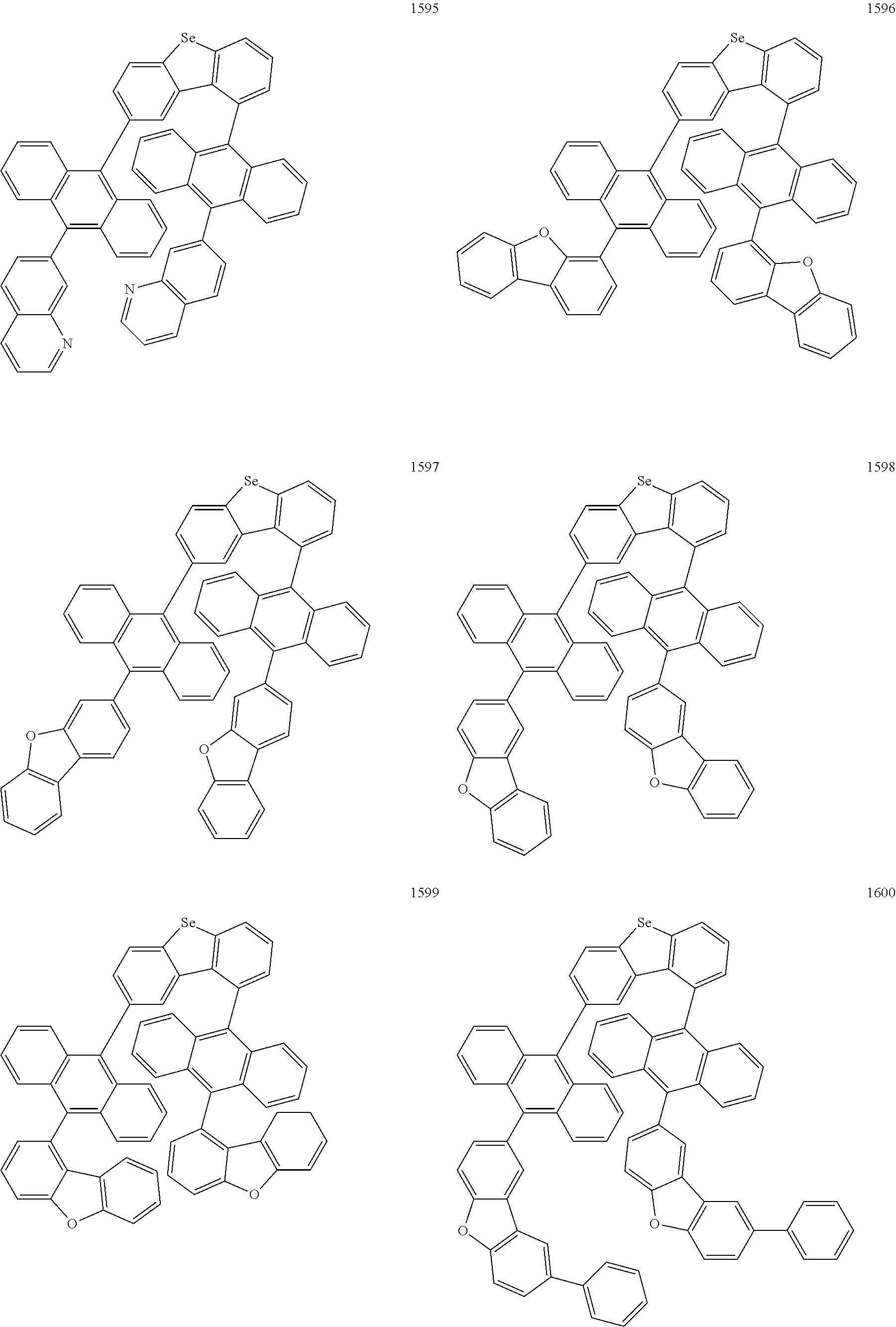

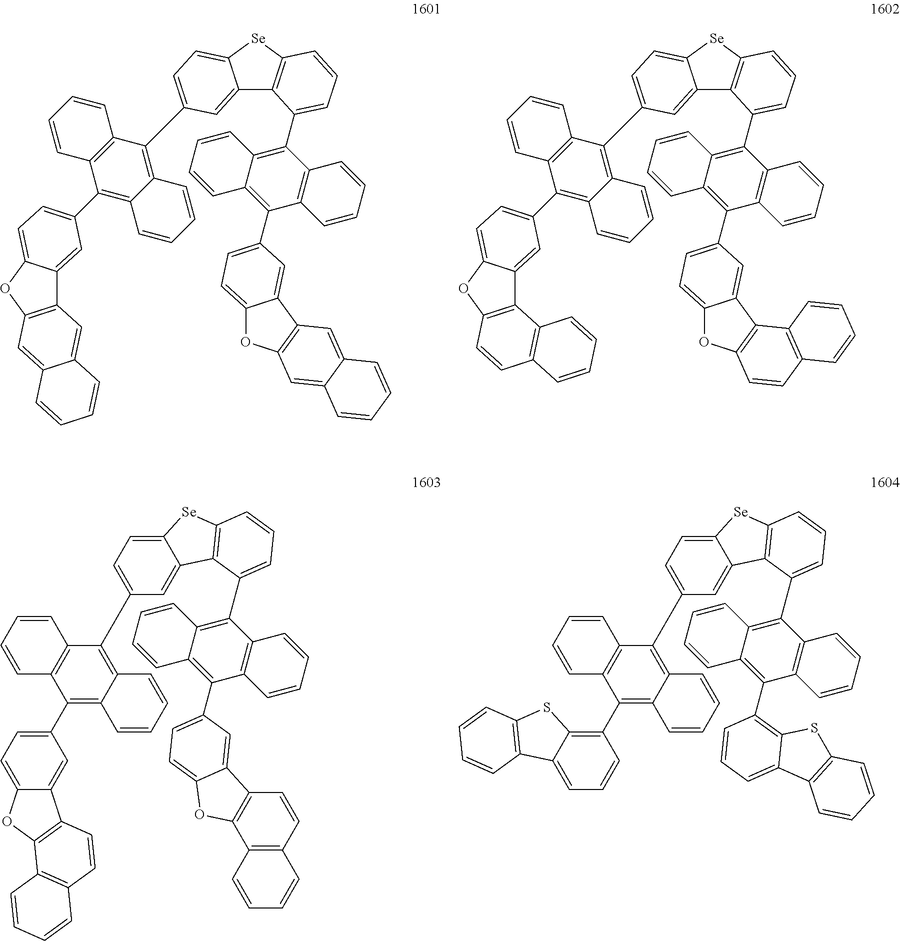

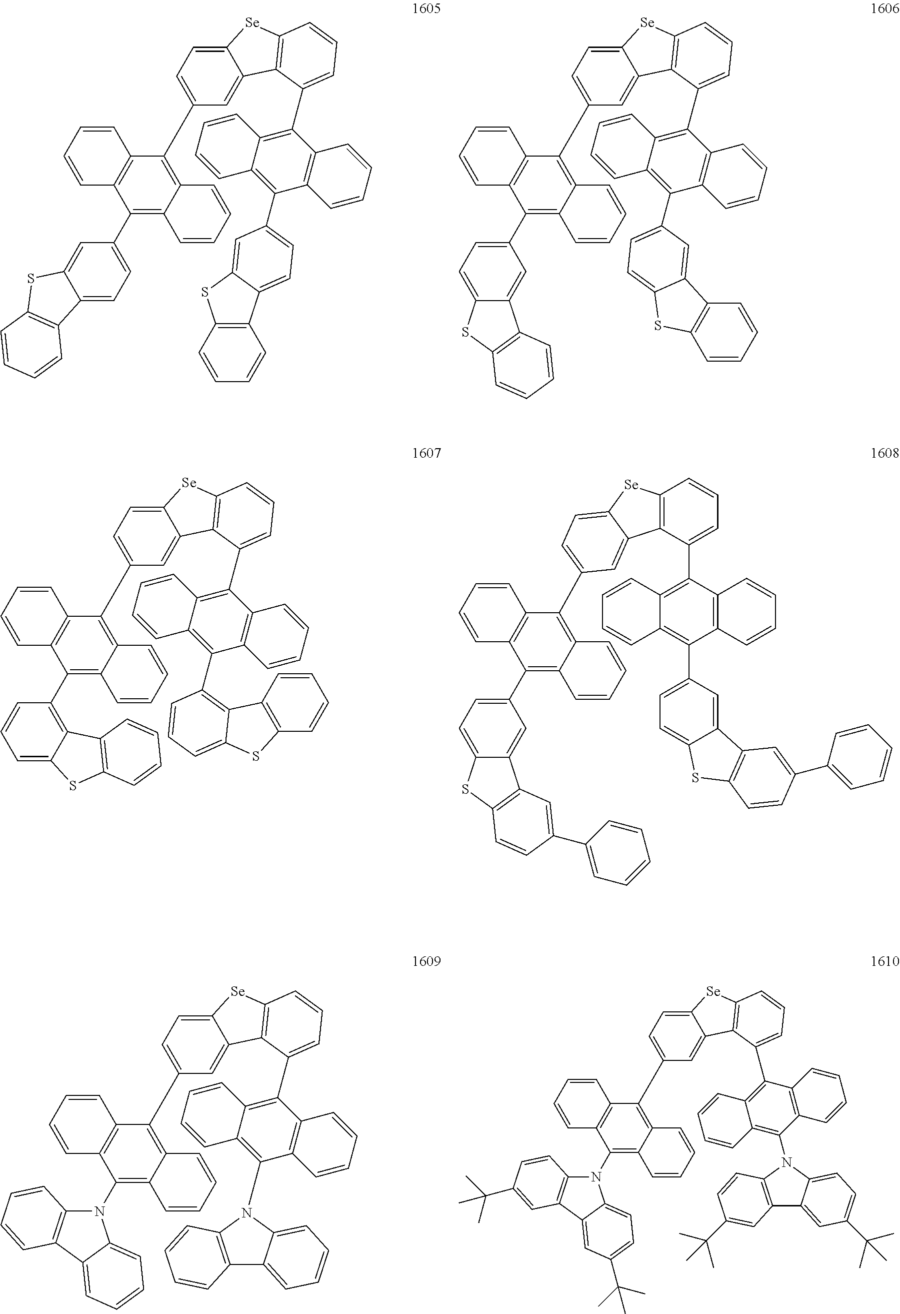

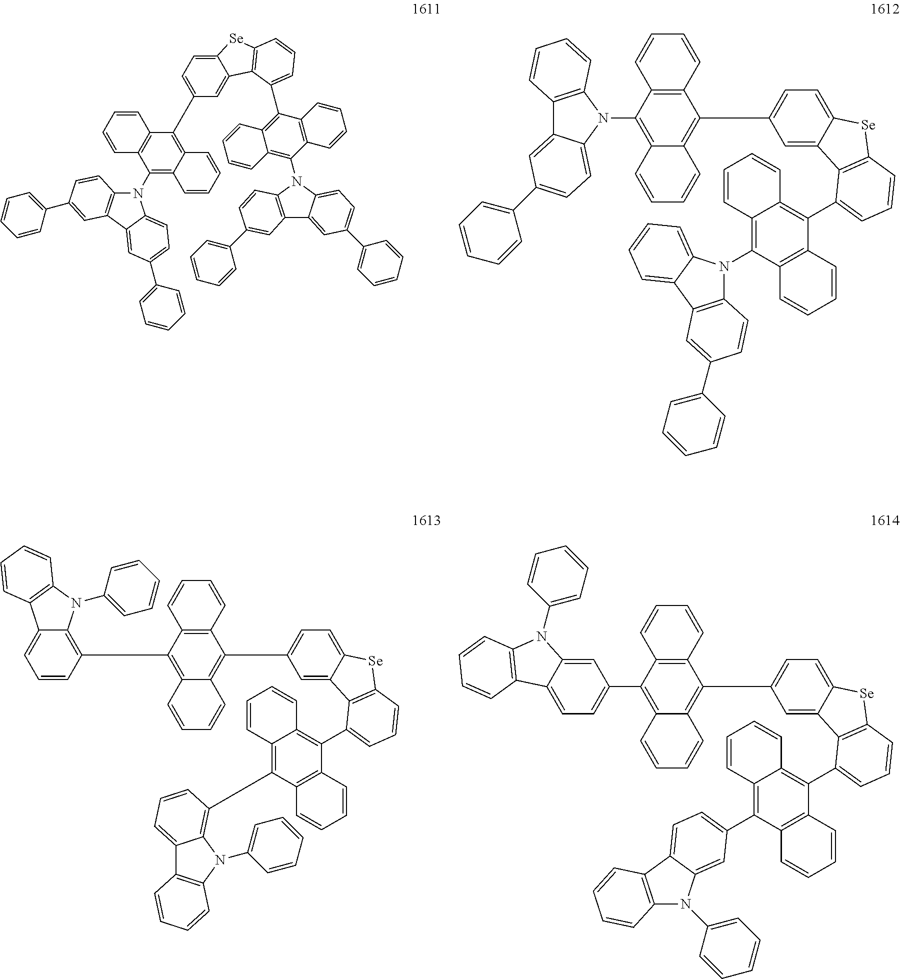

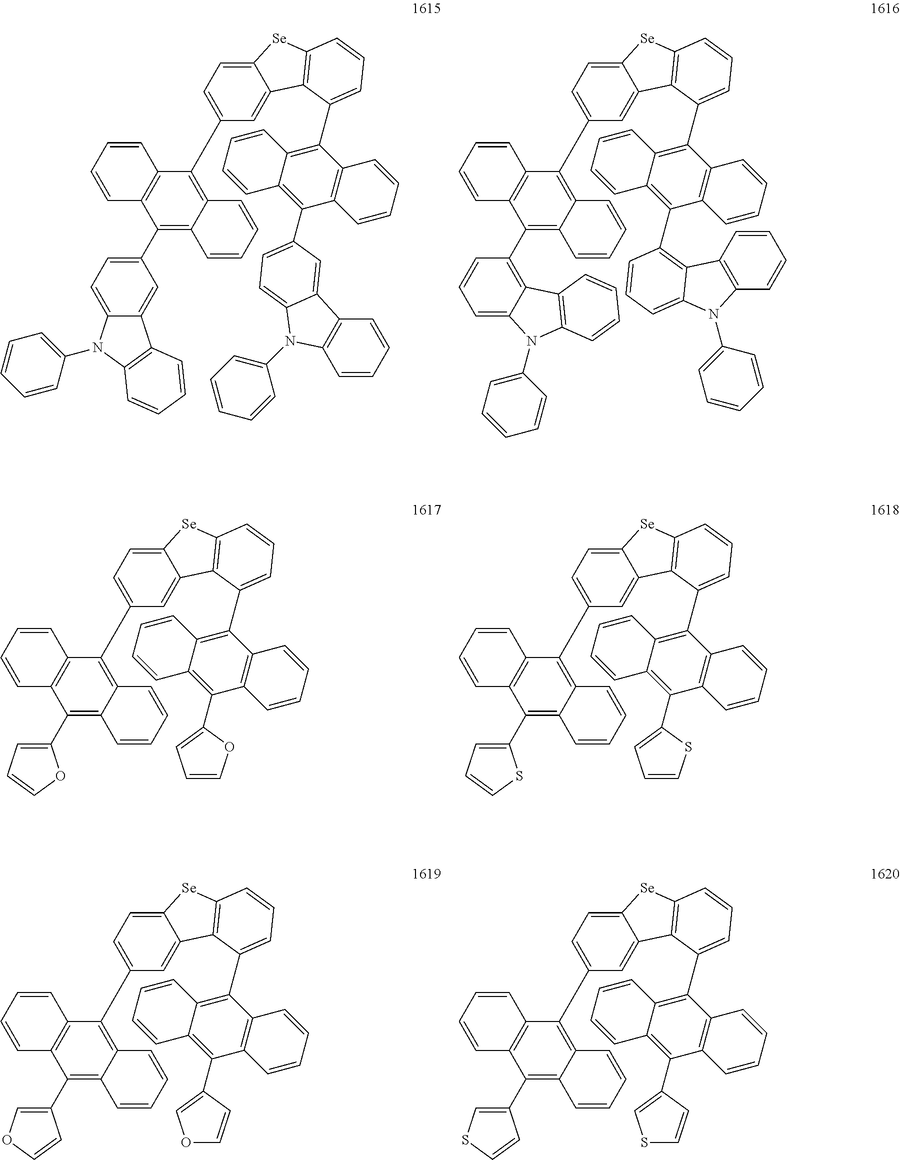

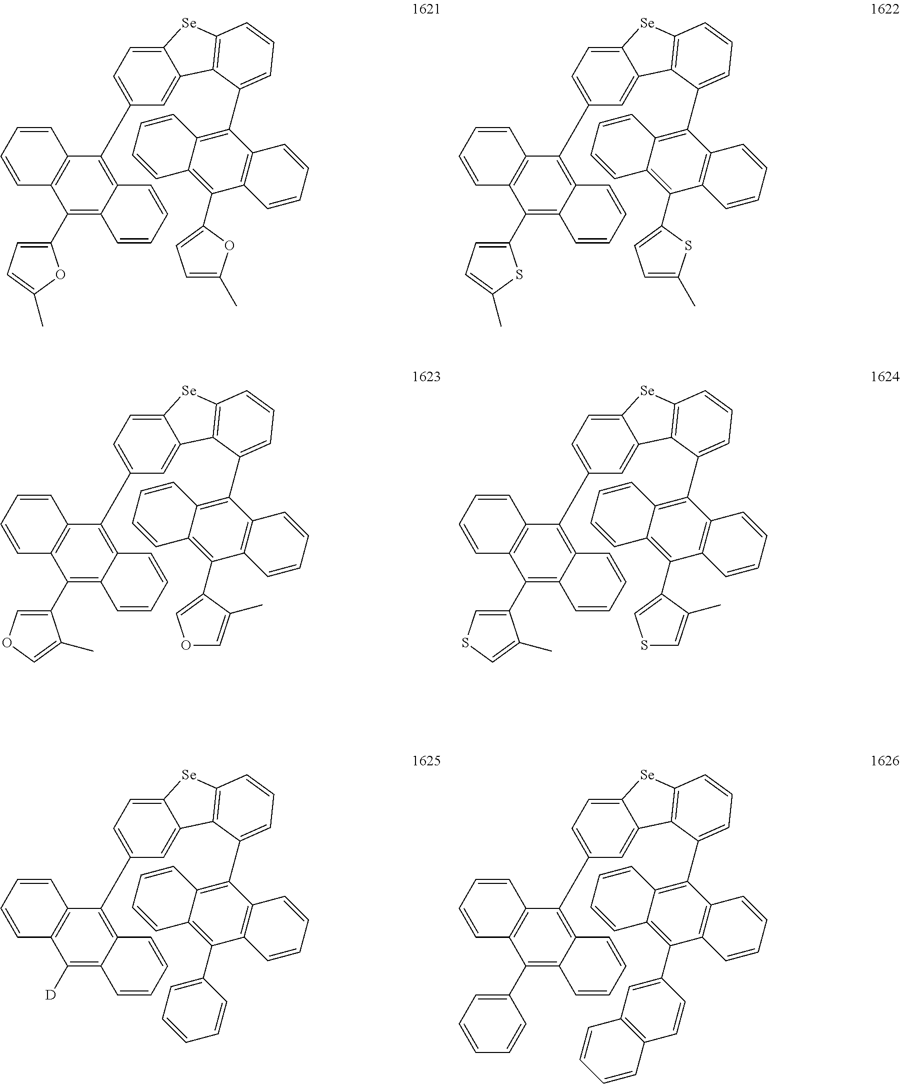

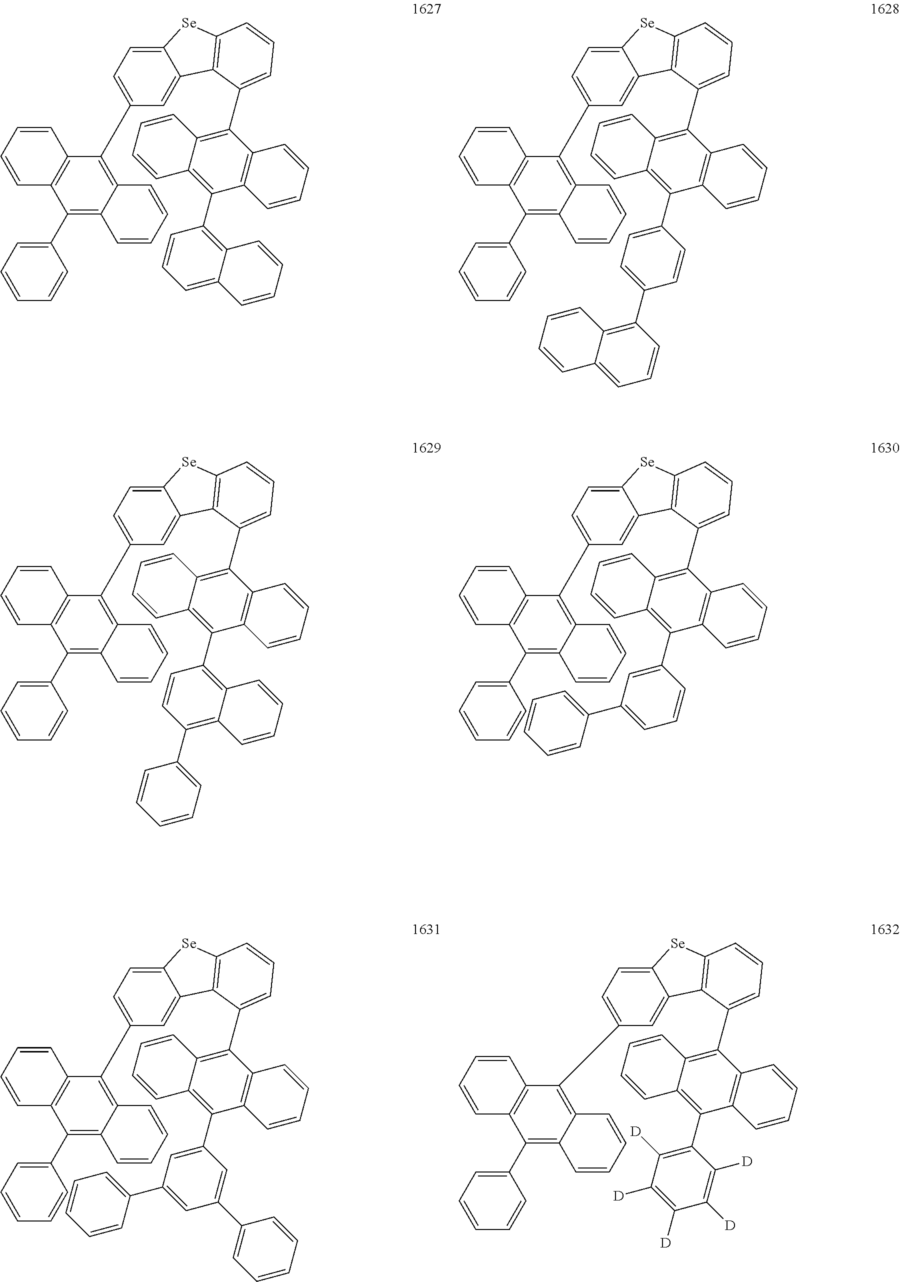

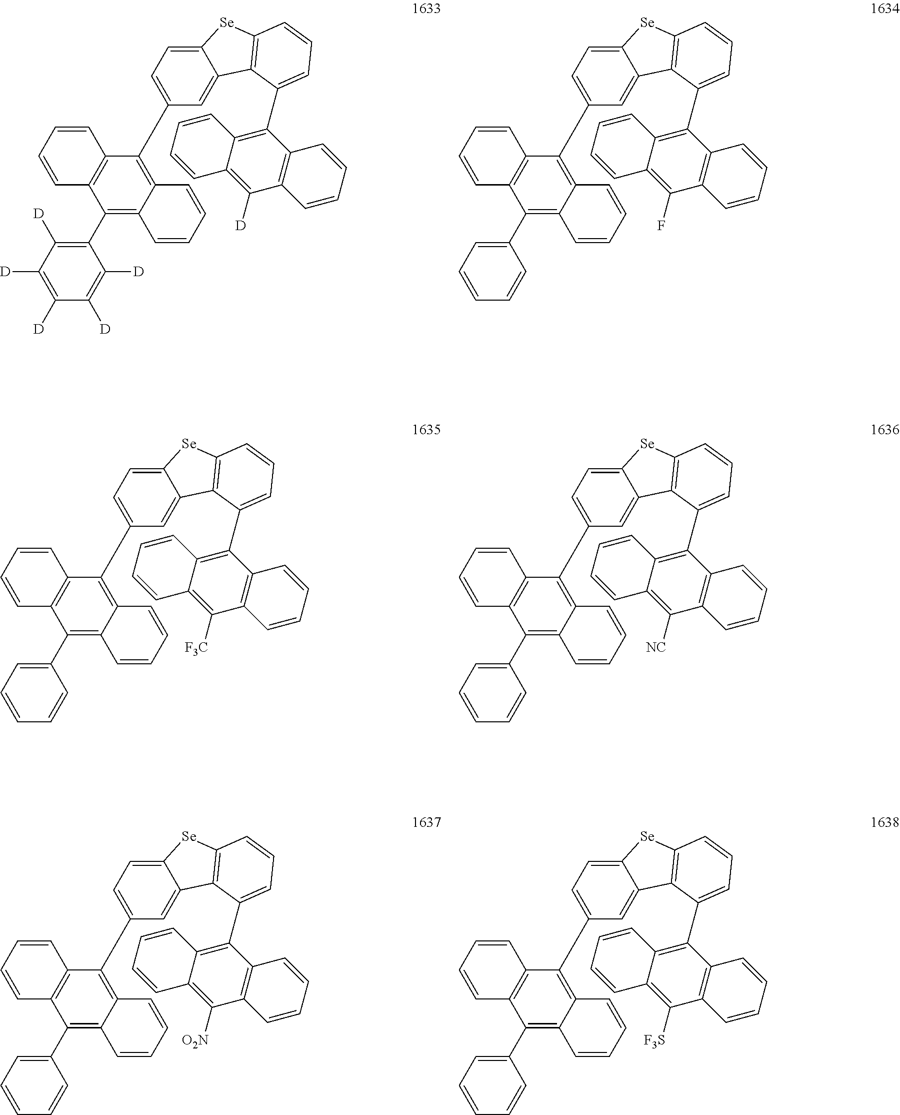

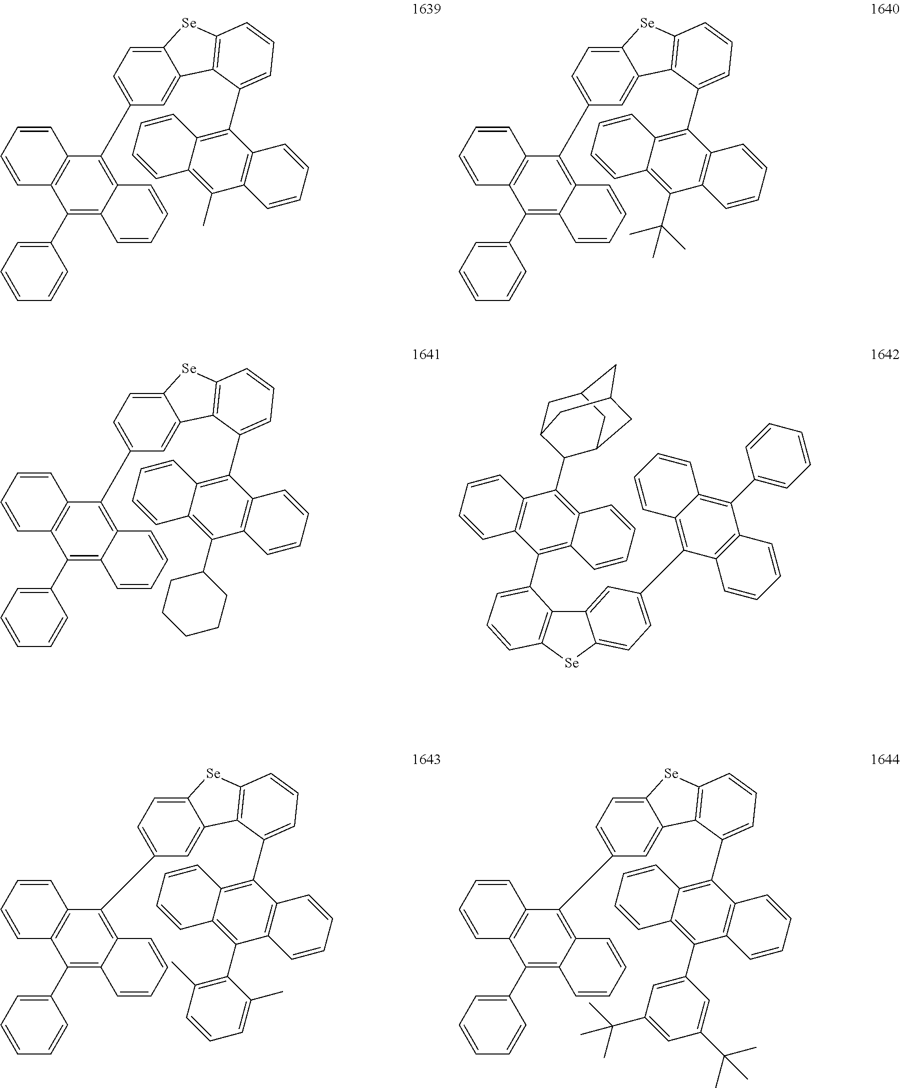

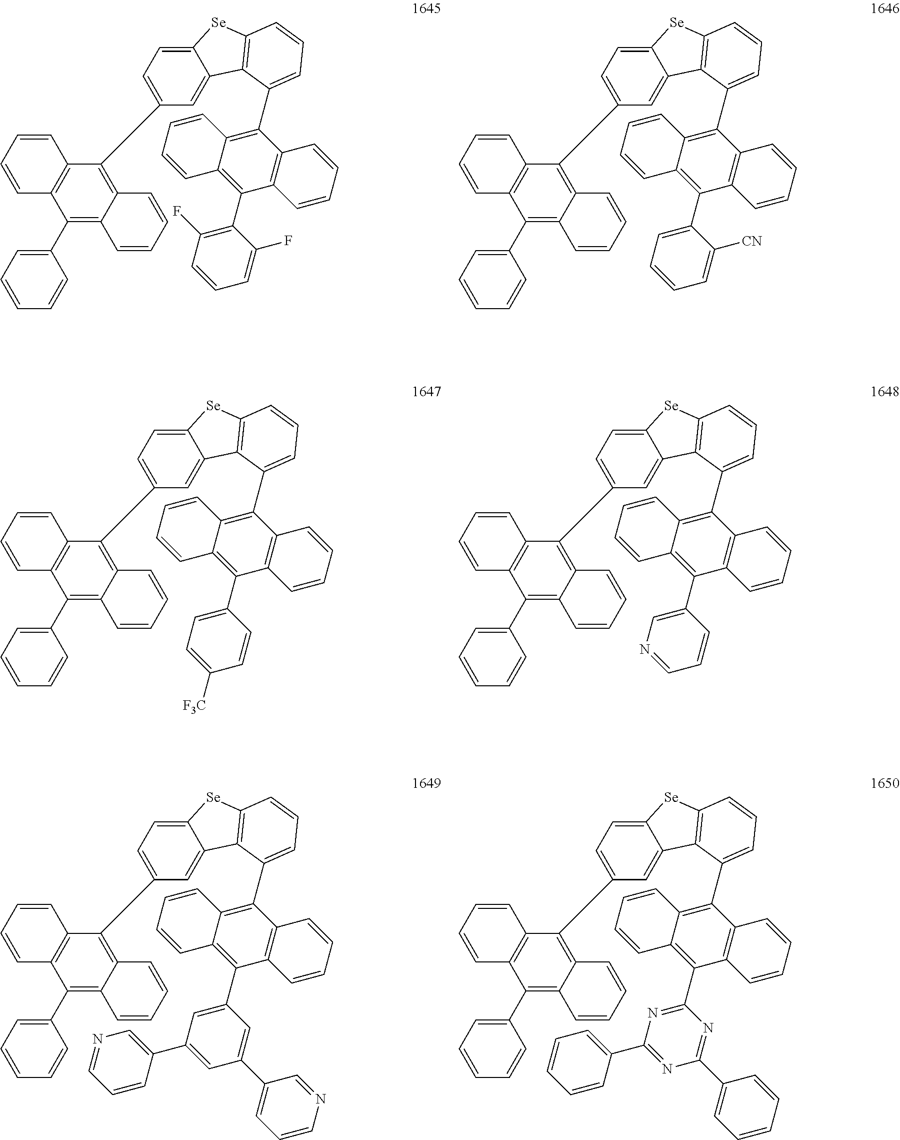

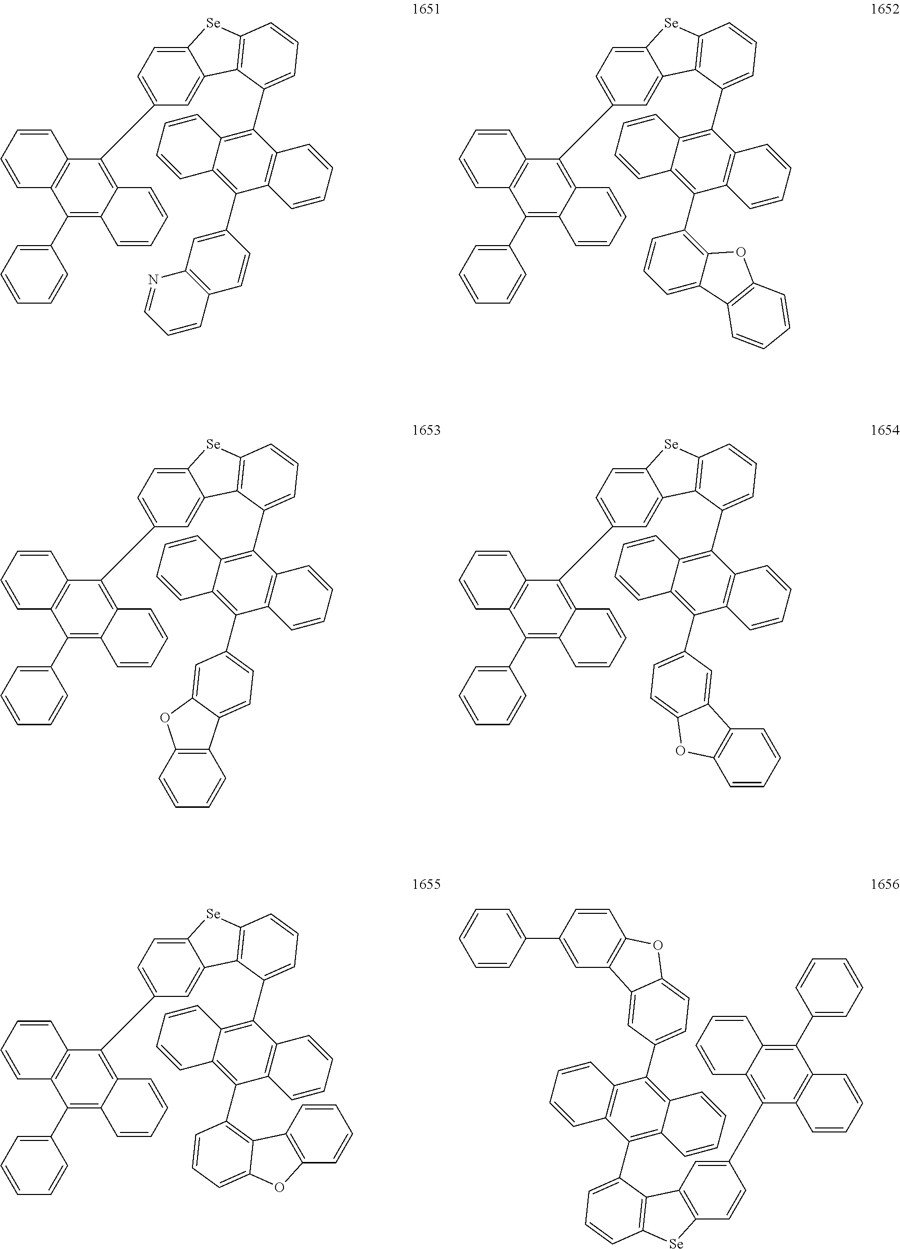

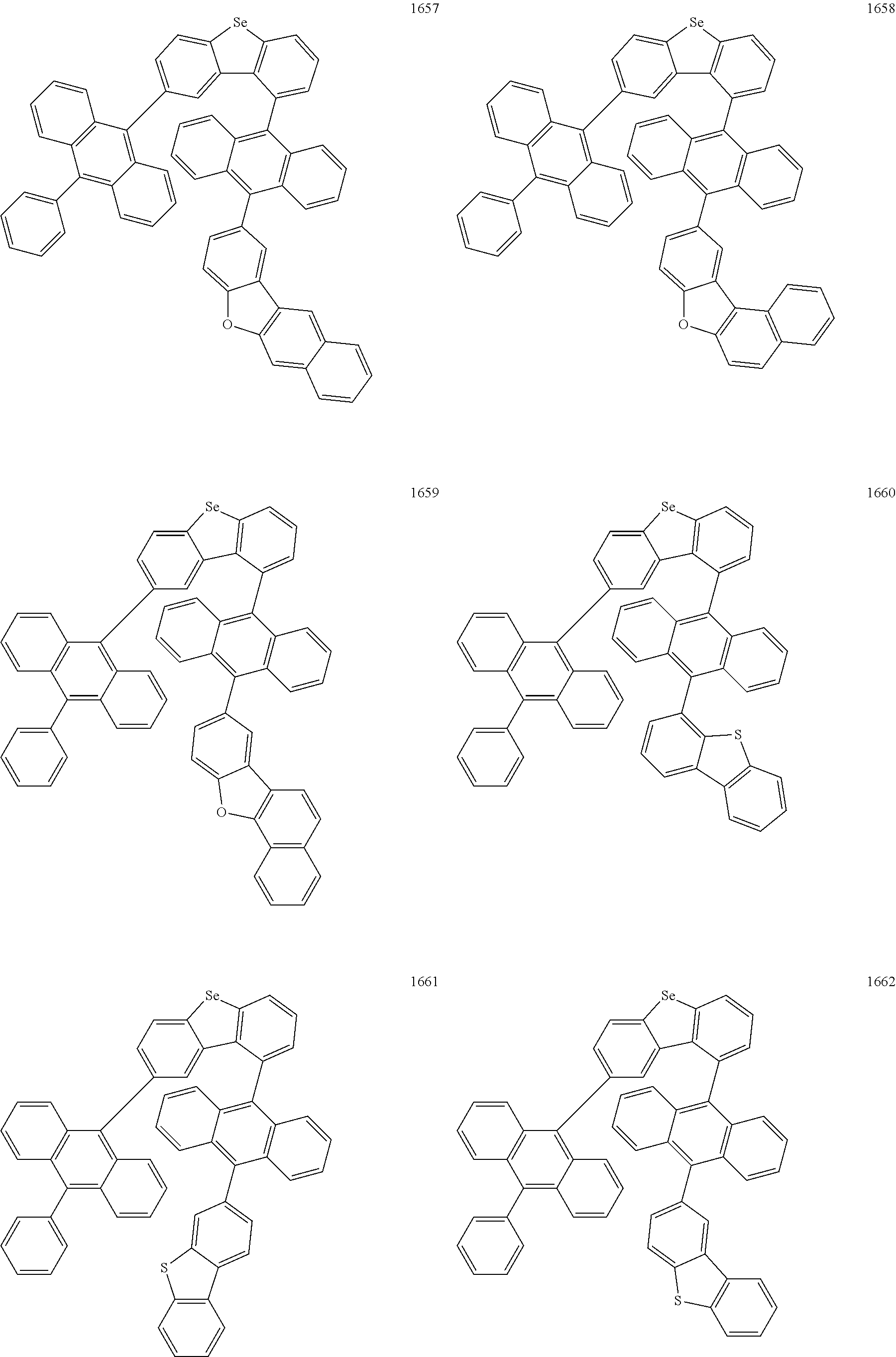

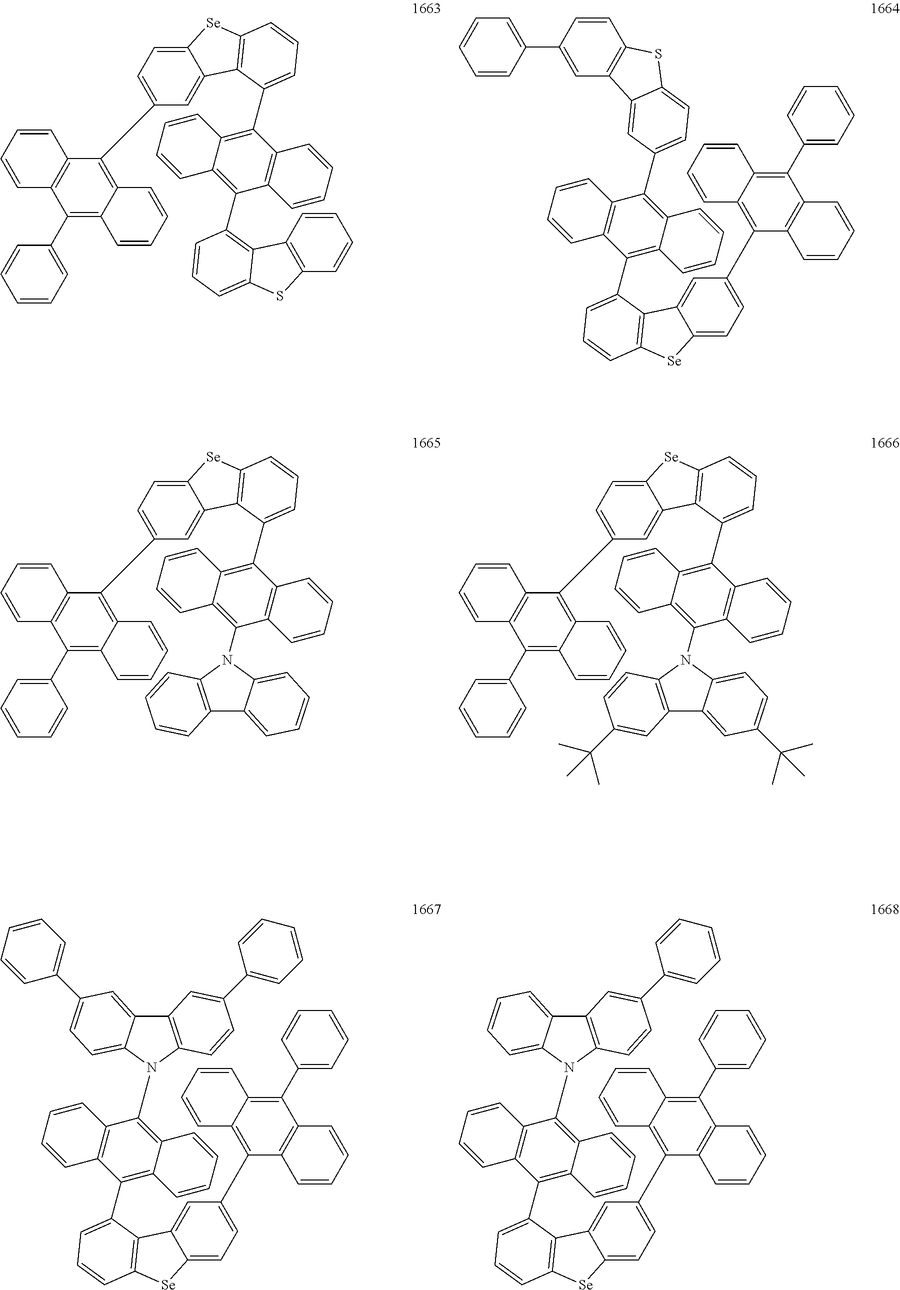

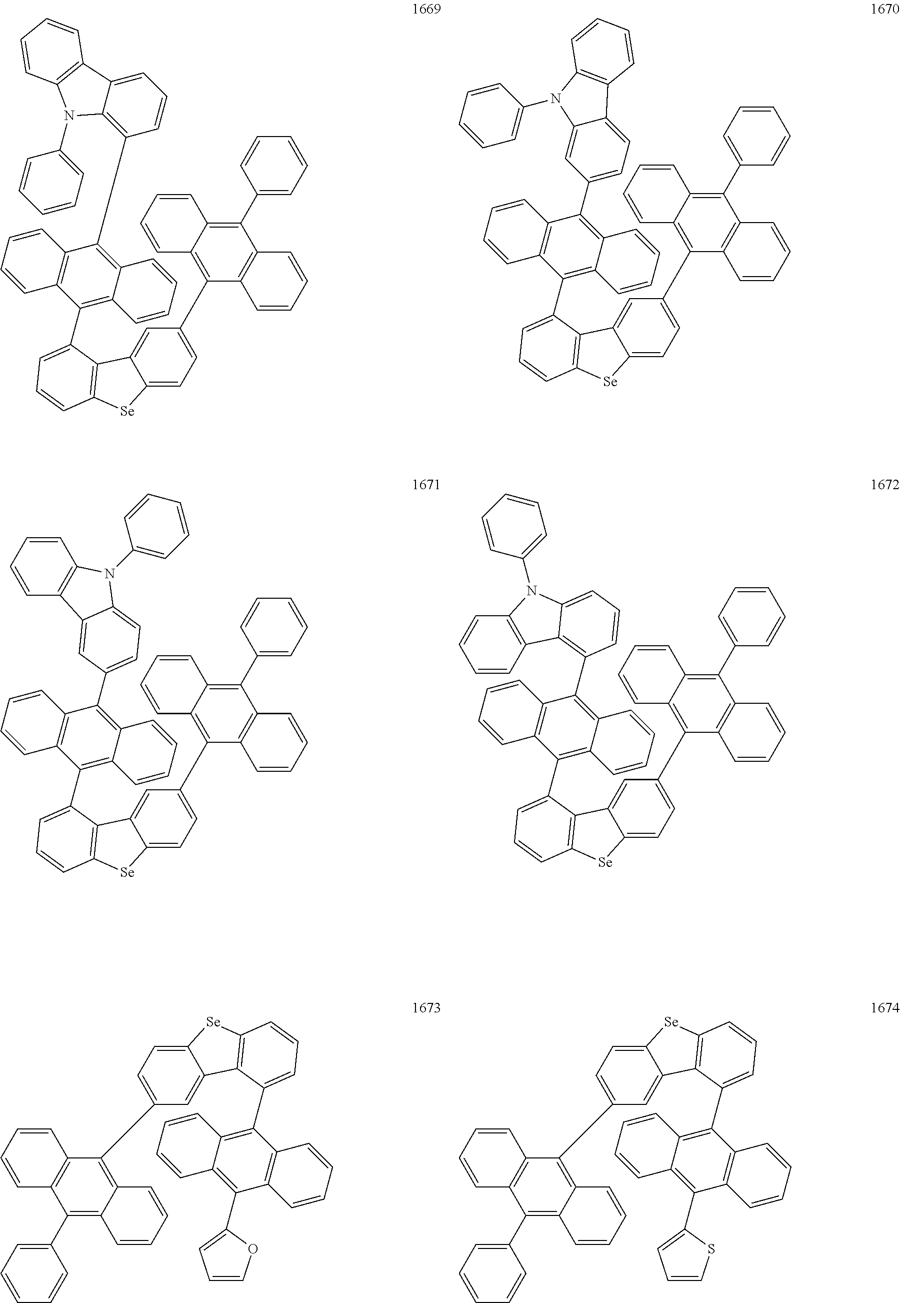

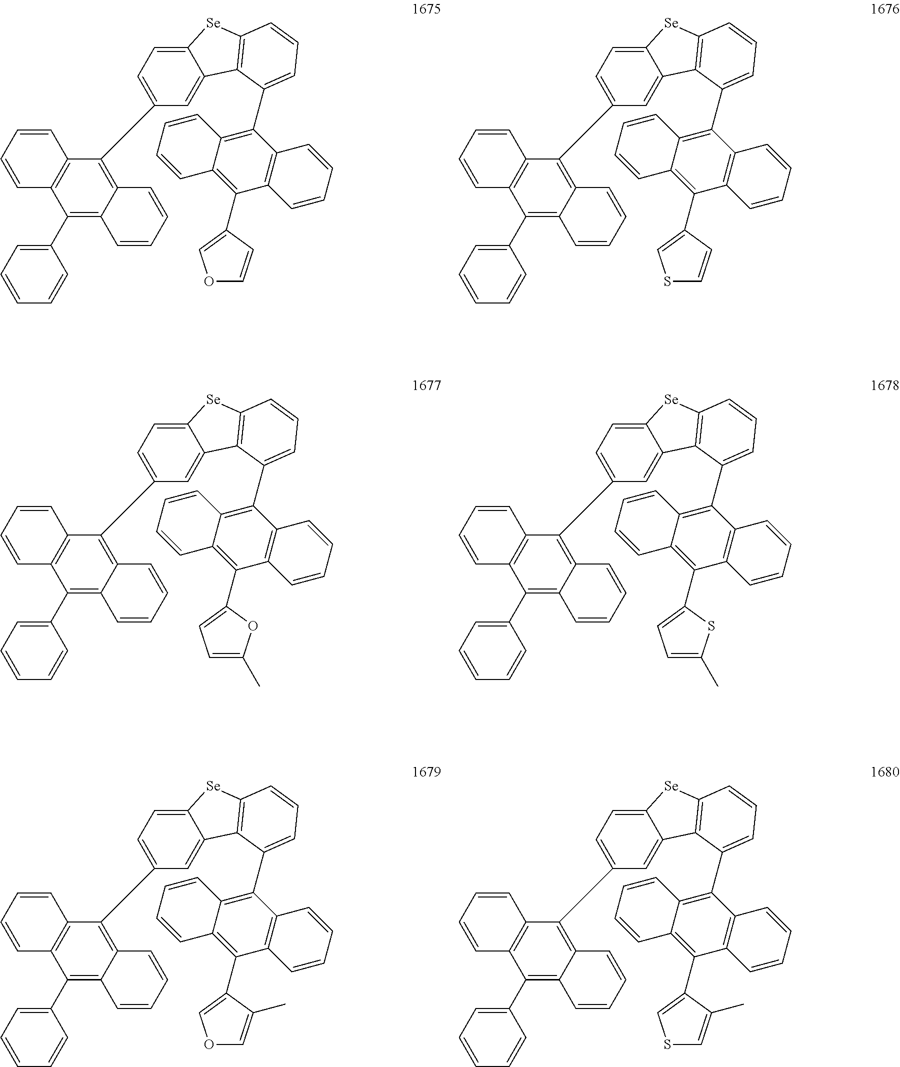

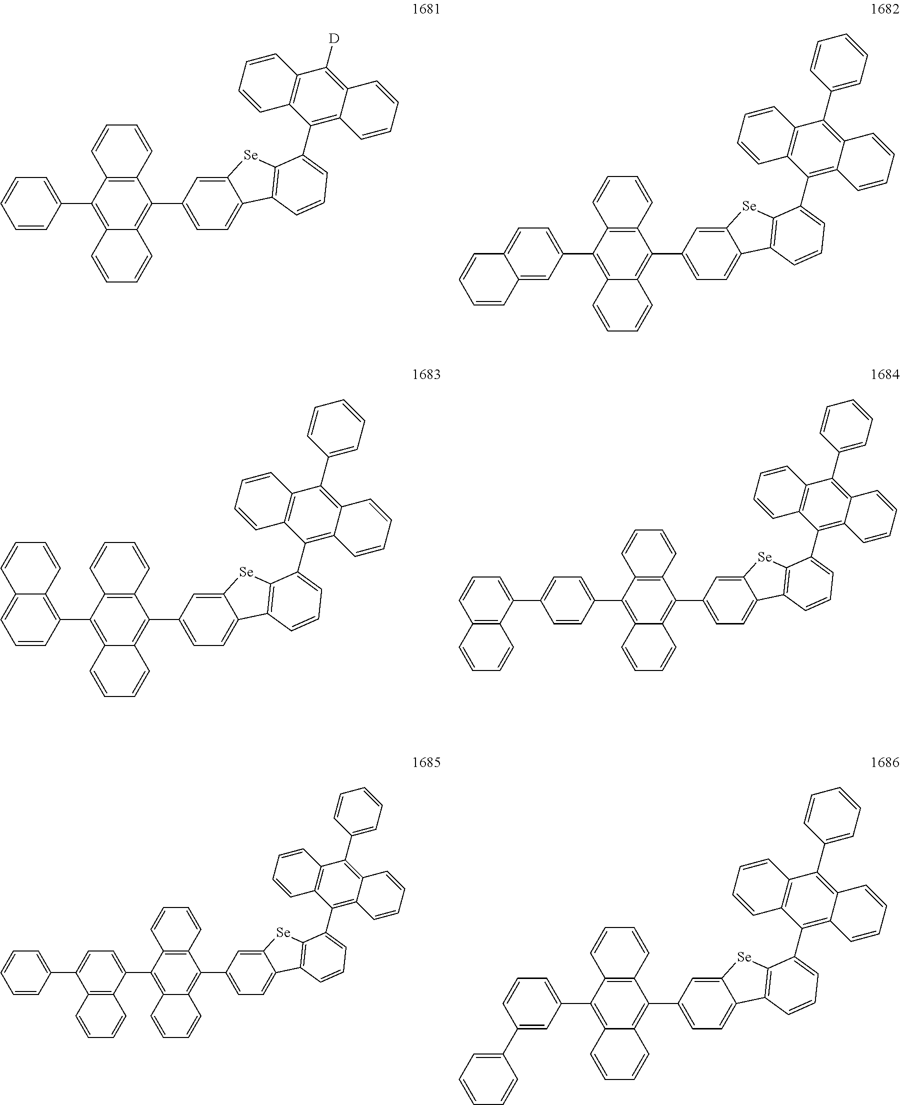

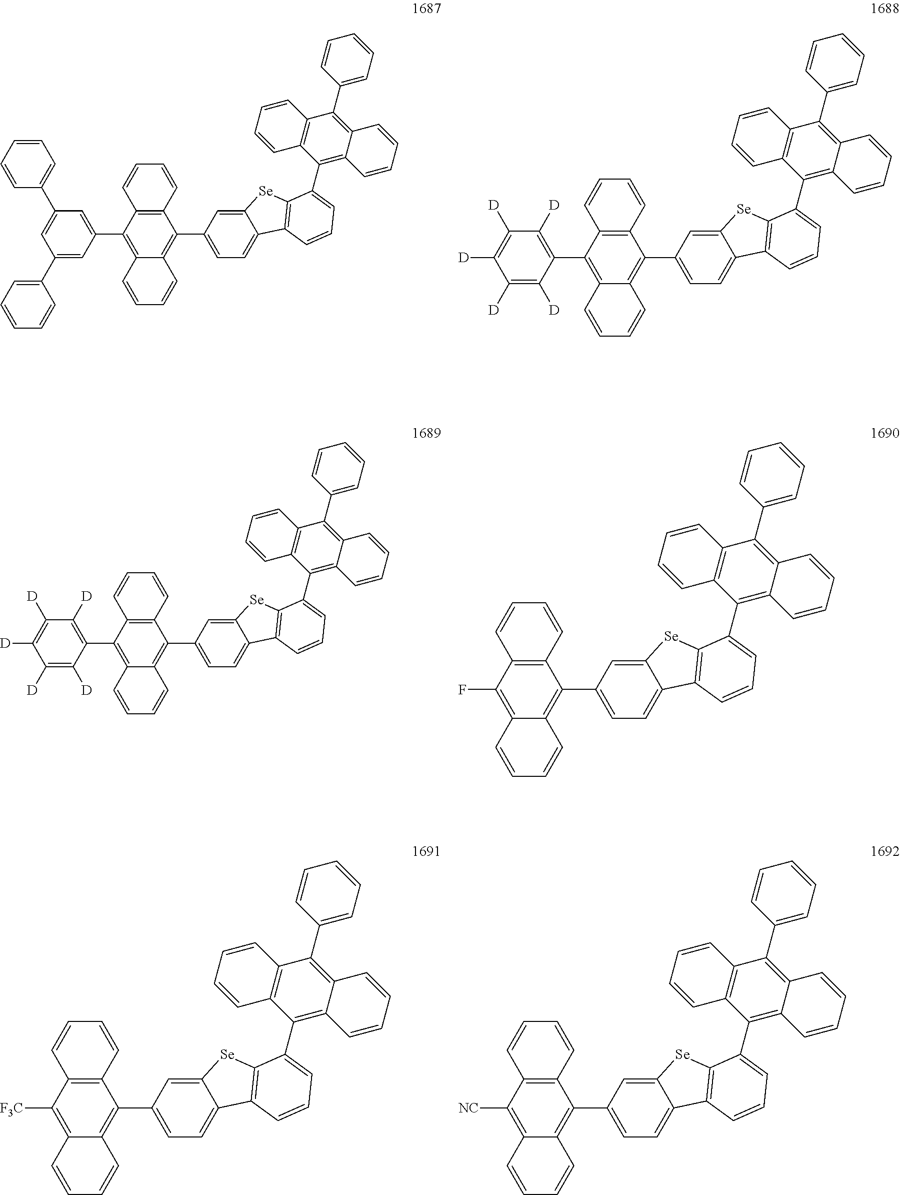

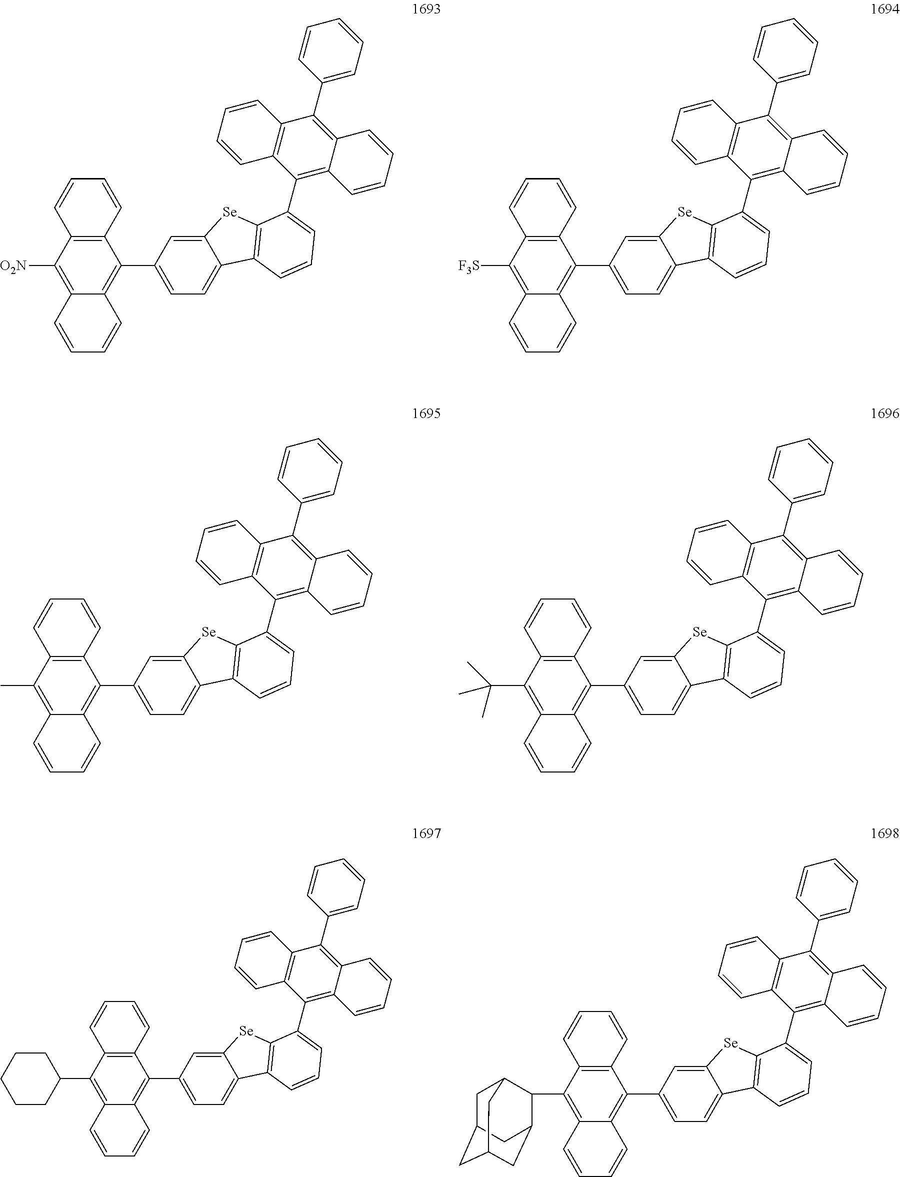

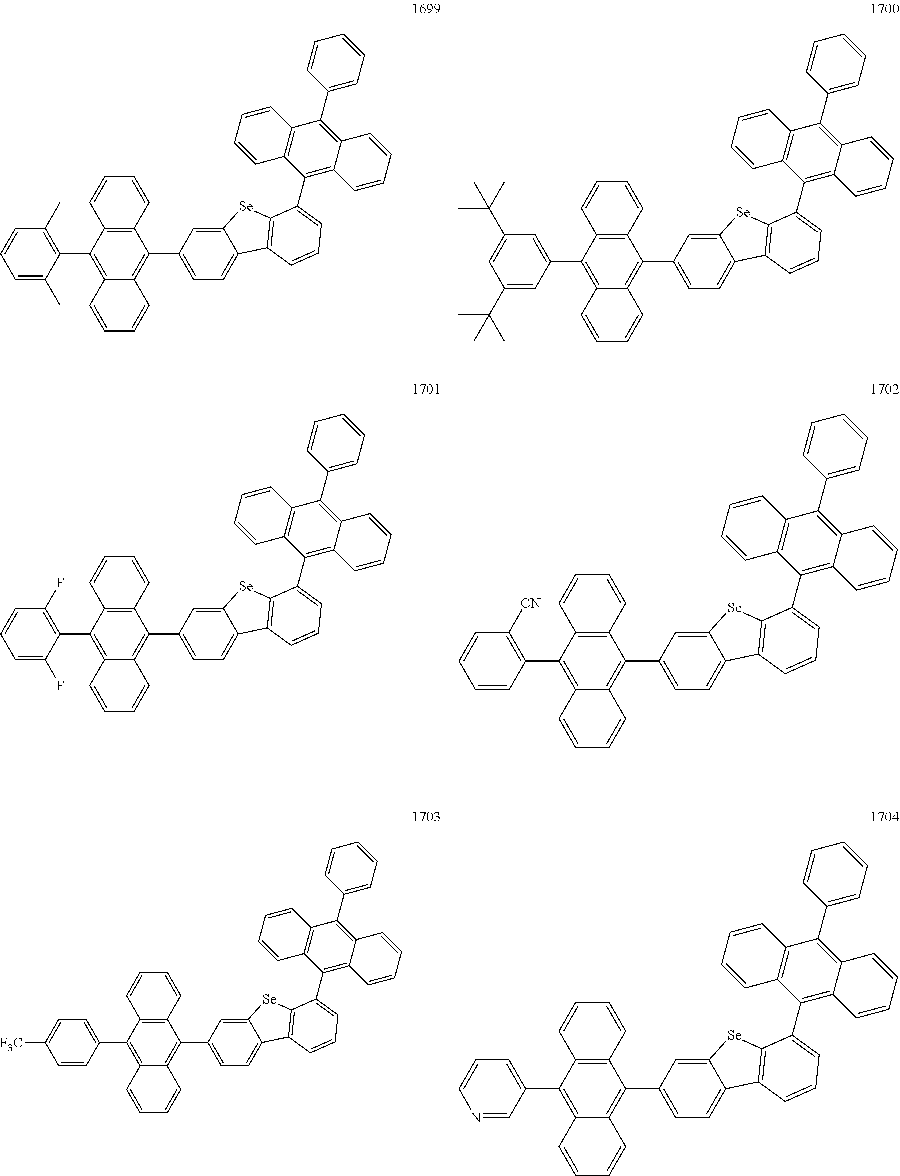

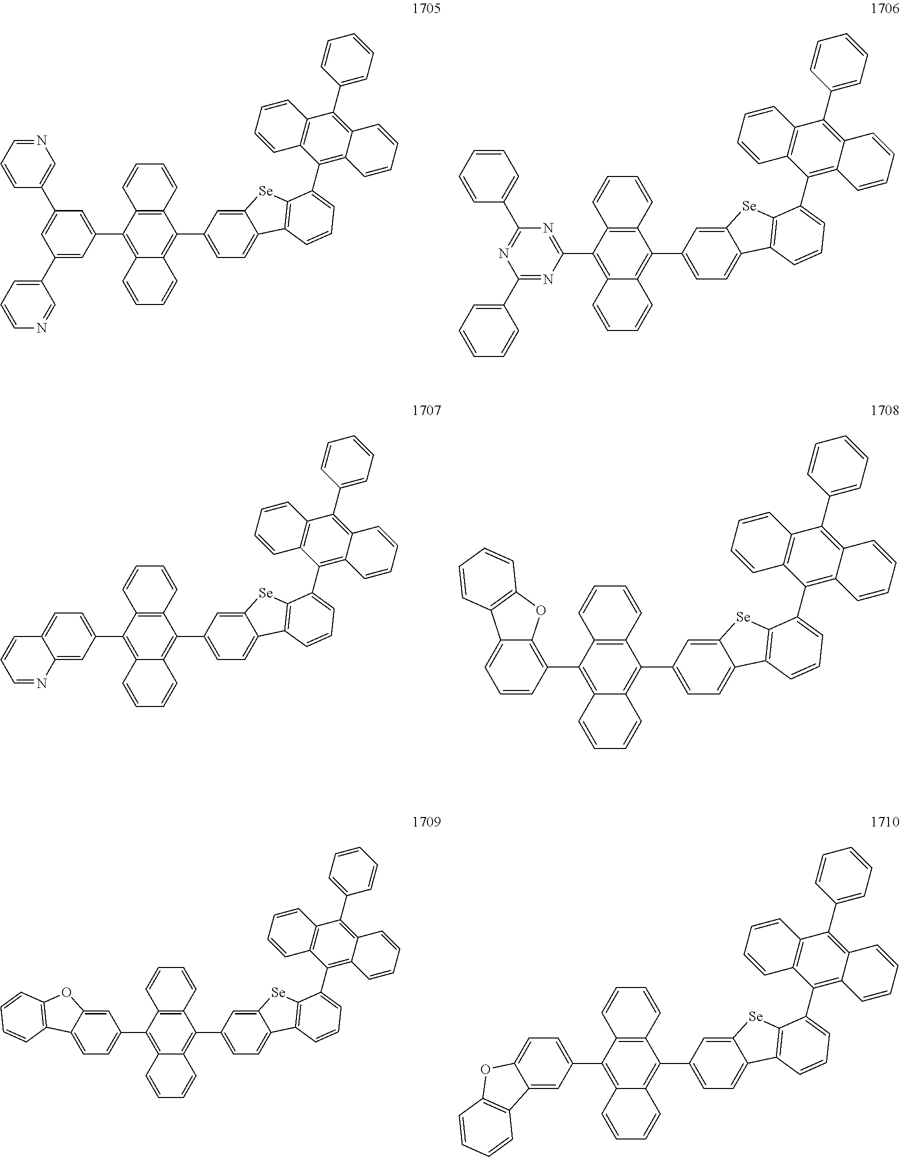

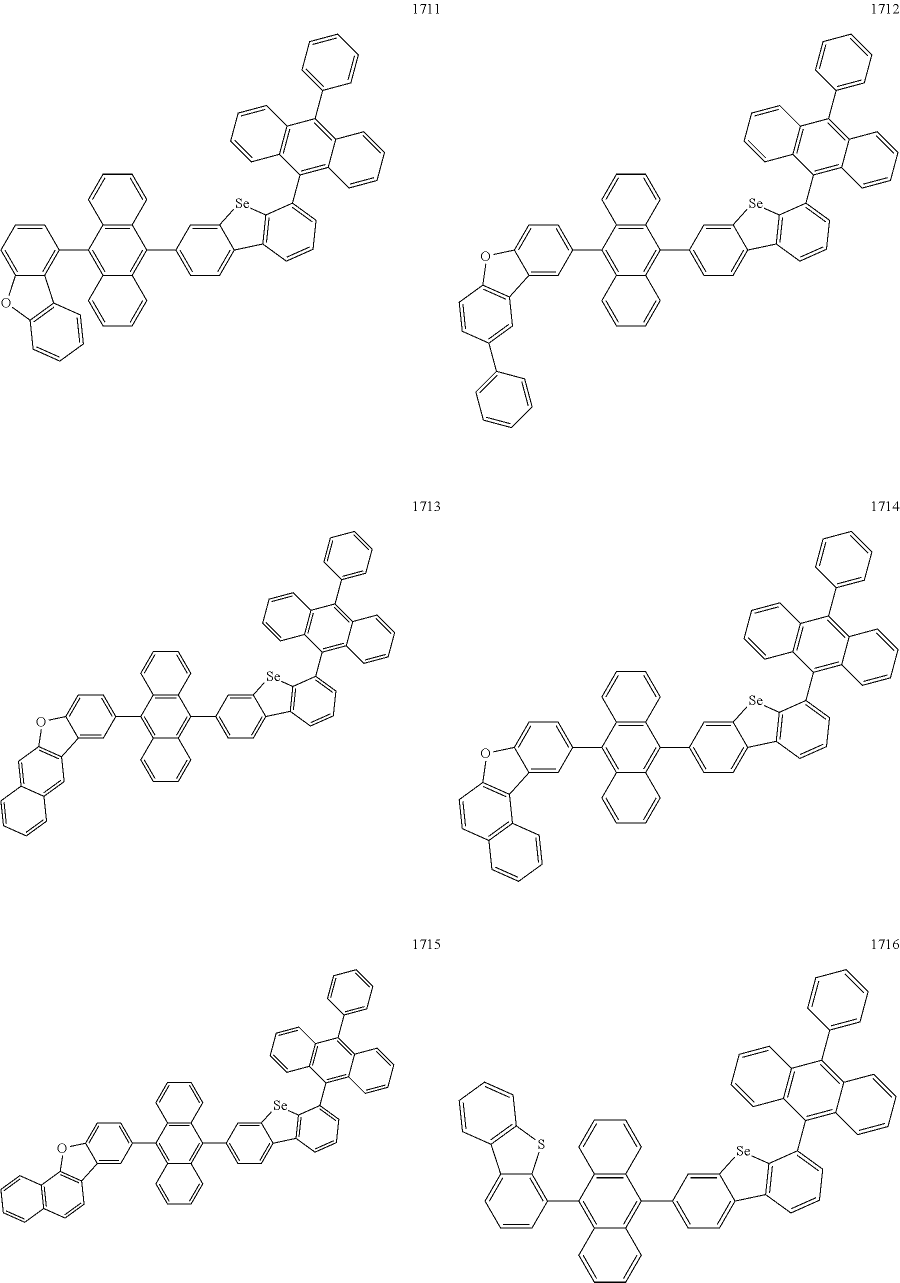

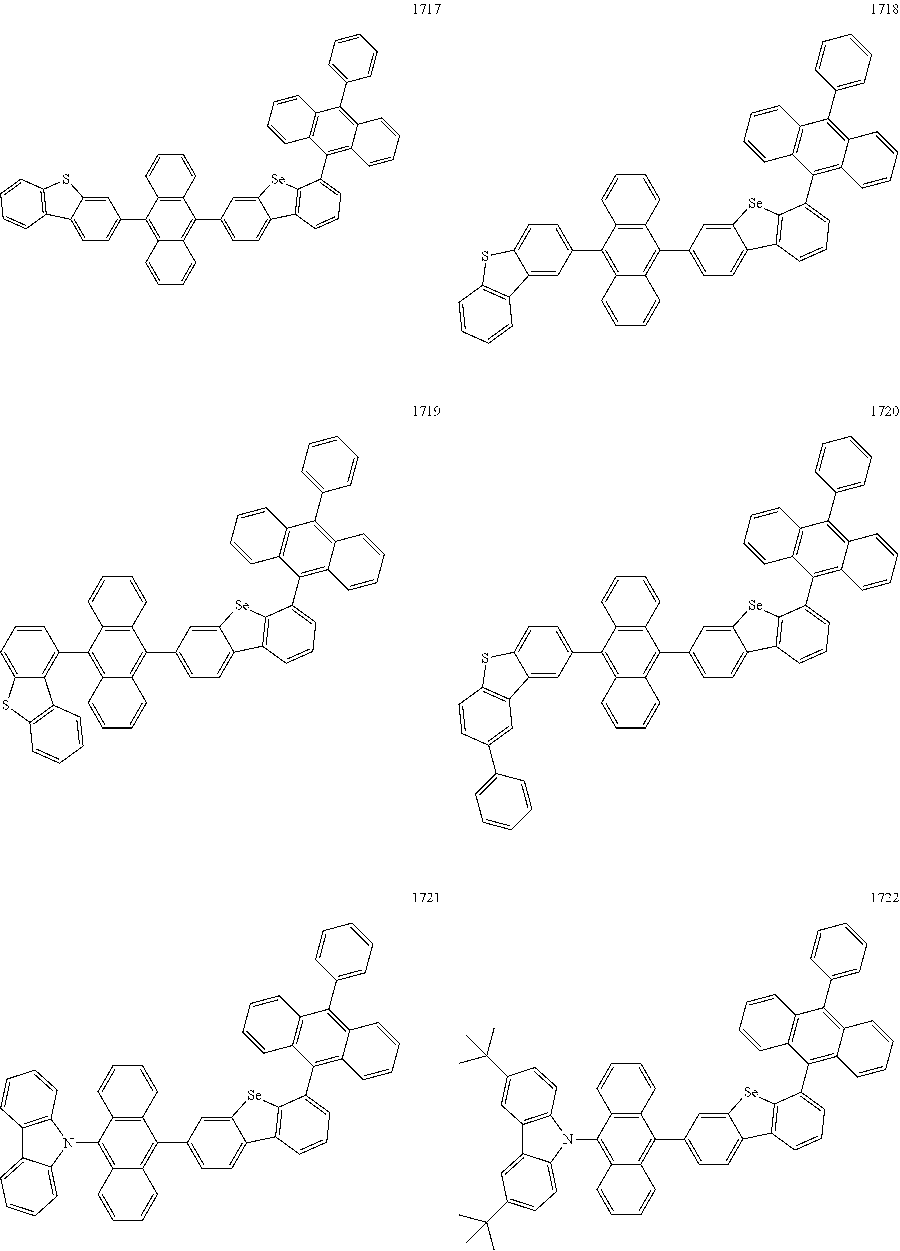

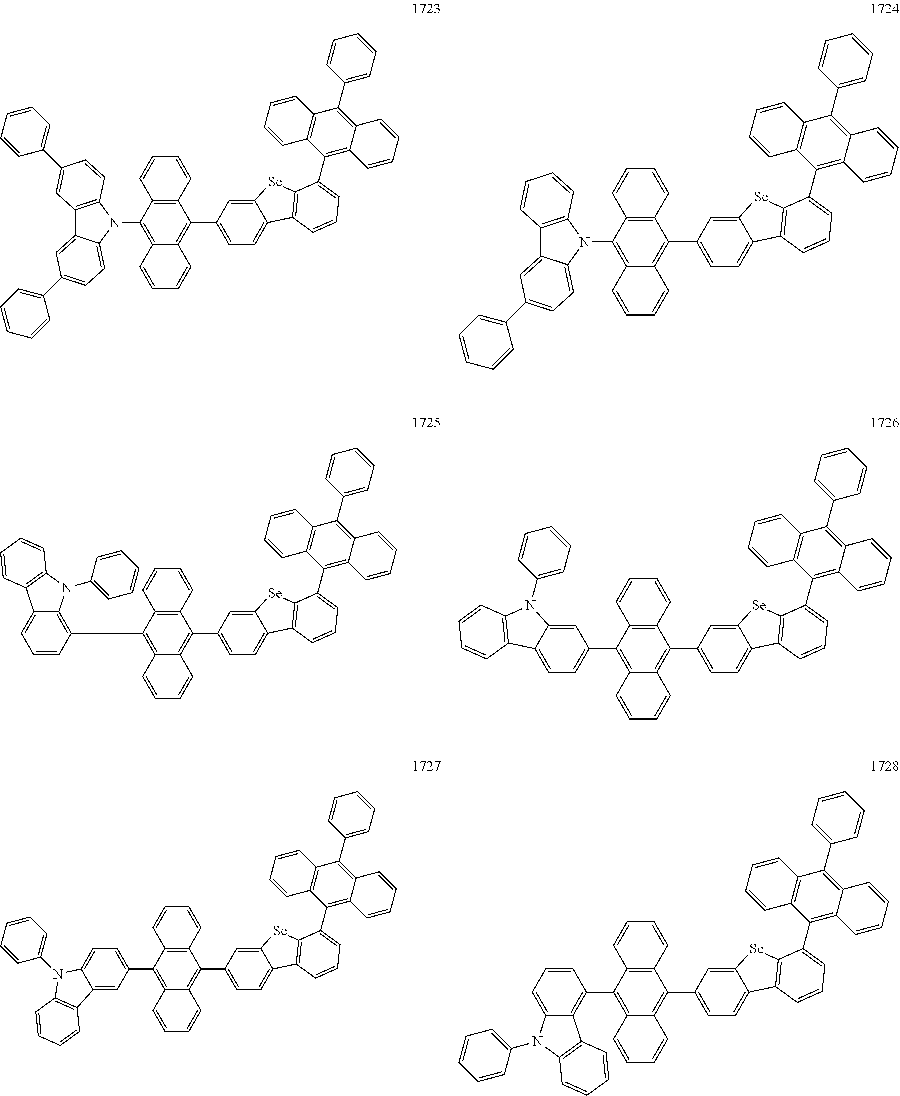

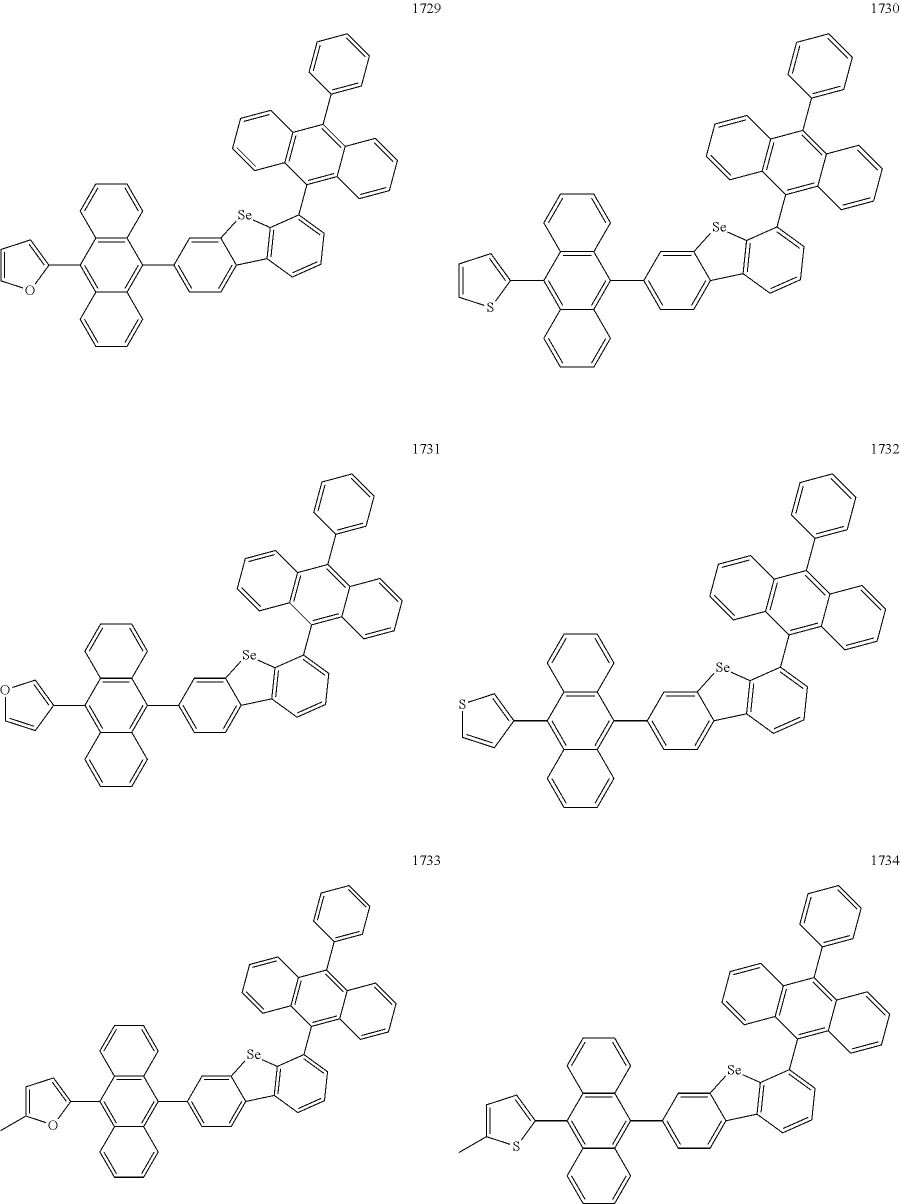

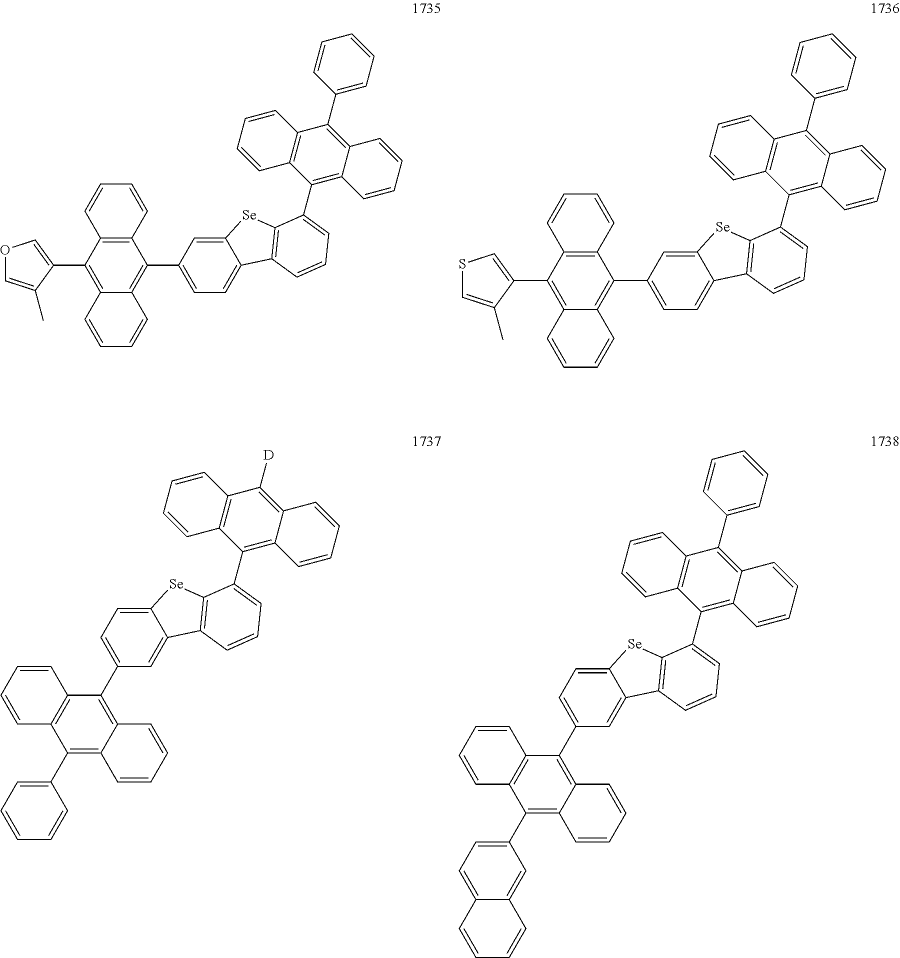 [SMILES: Cc1cocc1-c1c2ccccc2c(-c2ccc3c(c2)[se]c2c(-c4c5ccccc5c(-c5ccccc5)c5ccccc45)cccc23)c2ccccc12 ; Cc1cscc1-c1c2ccccc2c(-c2ccc3c(c2)[se]c2c(-c4c5ccccc5c(-c5ccccc5)c5ccccc45)cccc23)c2ccccc12 ; [2H]c1c2ccccc2c(-c2cccc3c2[se]c2ccc(-c4c5ccccc5c(-c5ccccc5)c5ccccc45)cc23)c2ccccc12 ; c1ccc(-c2c3ccccc3c(-c3cccc4c3[se]c3ccc(-c5c6ccccc6c(-c6ccc7ccccc7c6)c6ccccc56)cc34)c3ccccc23)cc1]

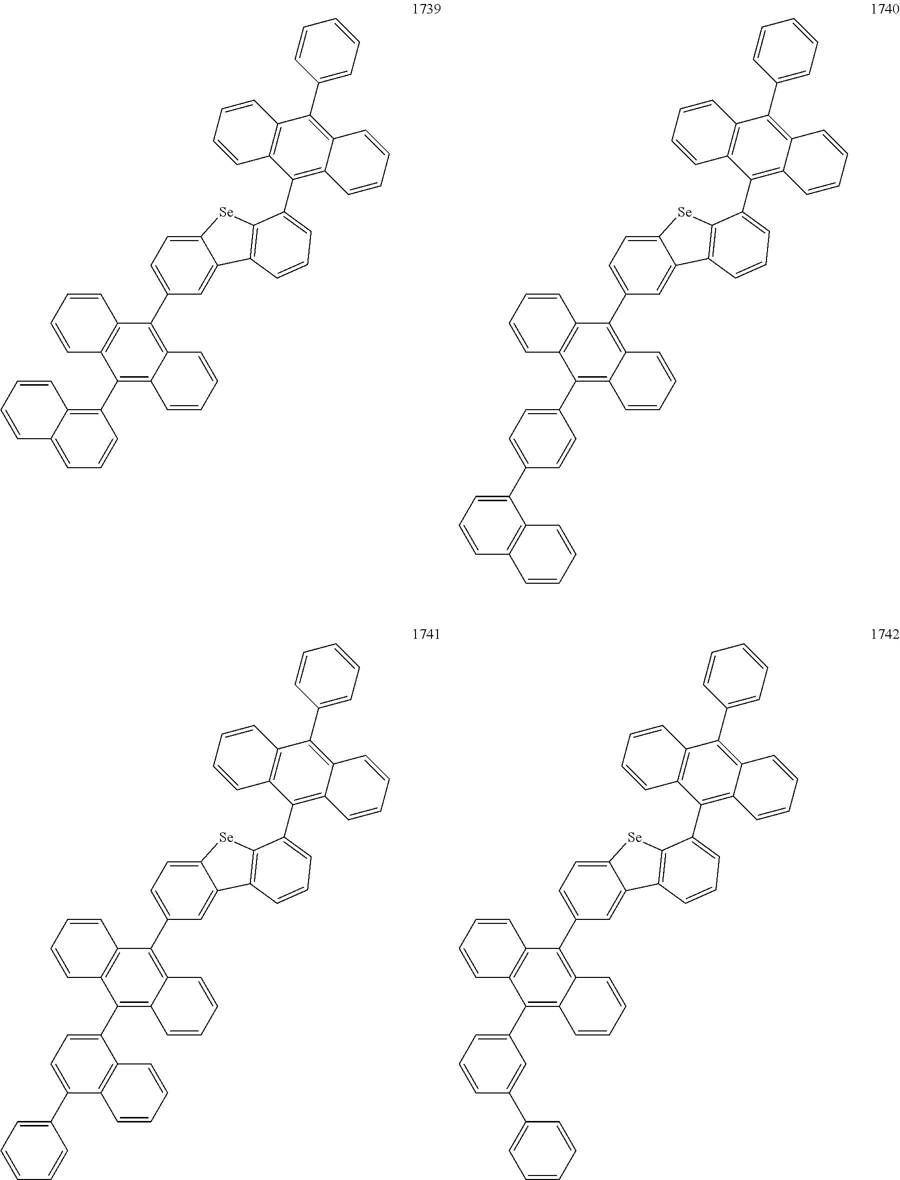 [SMILES: c1ccc(-c2c3ccccc3c(-c3cccc4c3[se]c3ccc(-c5c6ccccc6c(-c6cccc7ccccc67)c6ccccc56)cc34)c3ccccc23)cc1 ; c1ccc(-c2c3ccccc3c(-c3cccc4c3[se]c3ccc(-c5c6ccccc6c(-c6ccc(-c7cccc8ccccc78)cc6)c6ccccc56)cc34)c3ccccc23)cc1 ; c1ccc(-c2ccc(-c3c4ccccc4c(-c4ccc5[se]c6c(-c7c8ccccc8c(-c8ccccc8)c8ccccc78)cccc6c5c4)c4ccccc34)c3ccccc23)cc1 ; c1ccc(-c2cccc(-c3c4ccccc4c(-c4ccc5[se]c6c(-c7c8ccccc8c(-c8ccccc8)c8ccccc78)cccc6c5c4)c4ccccc34)c2)cc1]

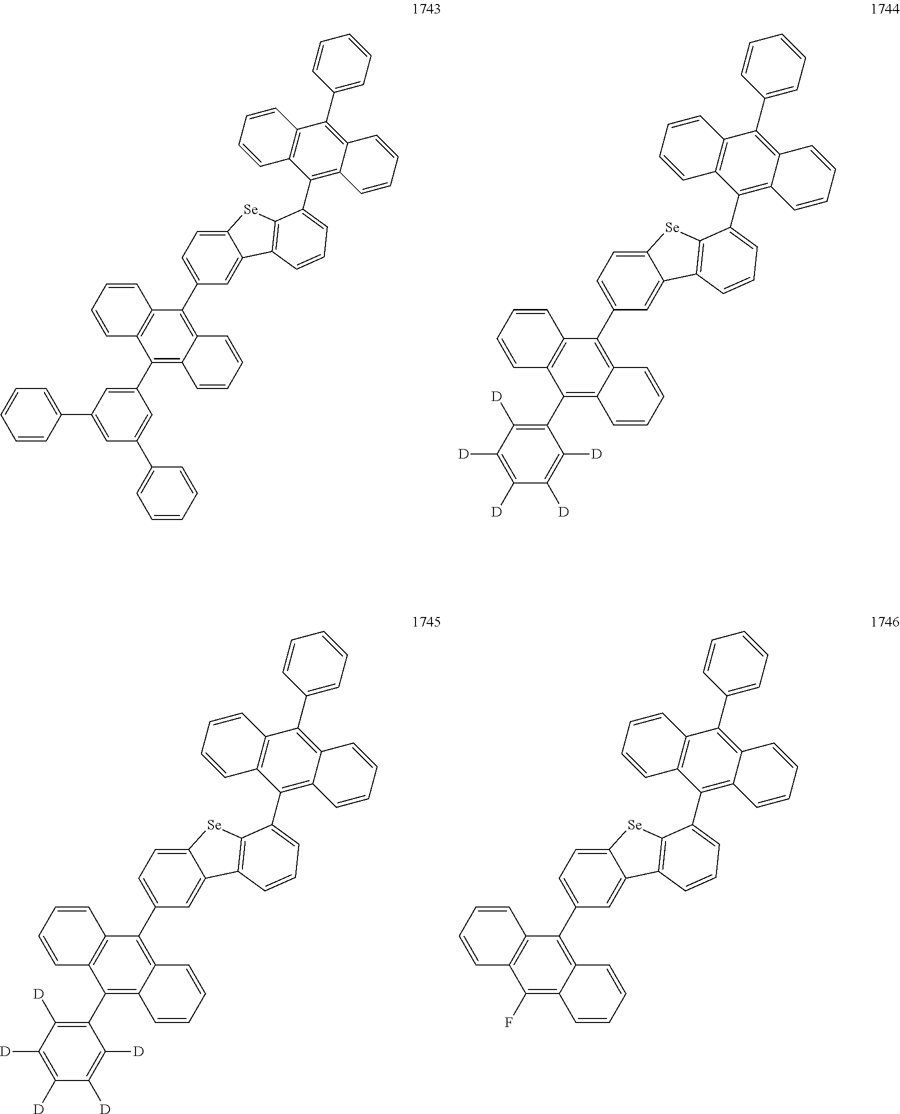 [SMILES: c1ccc(-c2cc(-c3ccccc3)cc(-c3c4ccccc4c(-c4ccc5[se]c6c(-c7c8ccccc8c(-c8ccccc8)c8ccccc78)cccc6c5c4)c4ccccc34)c2)cc1 ; [2H]c1c([2H])c([2H])c(-c2c3ccccc3c(-c3ccc4[se]c5c(-c6c7ccccc7c(-c7ccccc7)c7ccccc67)cccc5c4c3)c3ccccc23)c([2H])c1[2H] ; [2H]c1c([2H])c([2H])c(-c2c3ccccc3c(-c3ccc4[se]c5c(-c6c7ccccc7c(-c7ccccc7)c7ccccc67)cccc5c4c3)c3ccccc23)c([2H])c1[2H] ; Fc1c2ccccc2c(-c2ccc3[se]c4c(-c5c6ccccc6c(-c6ccccc6)c6ccccc56)cccc4c3c2)c2ccccc12]

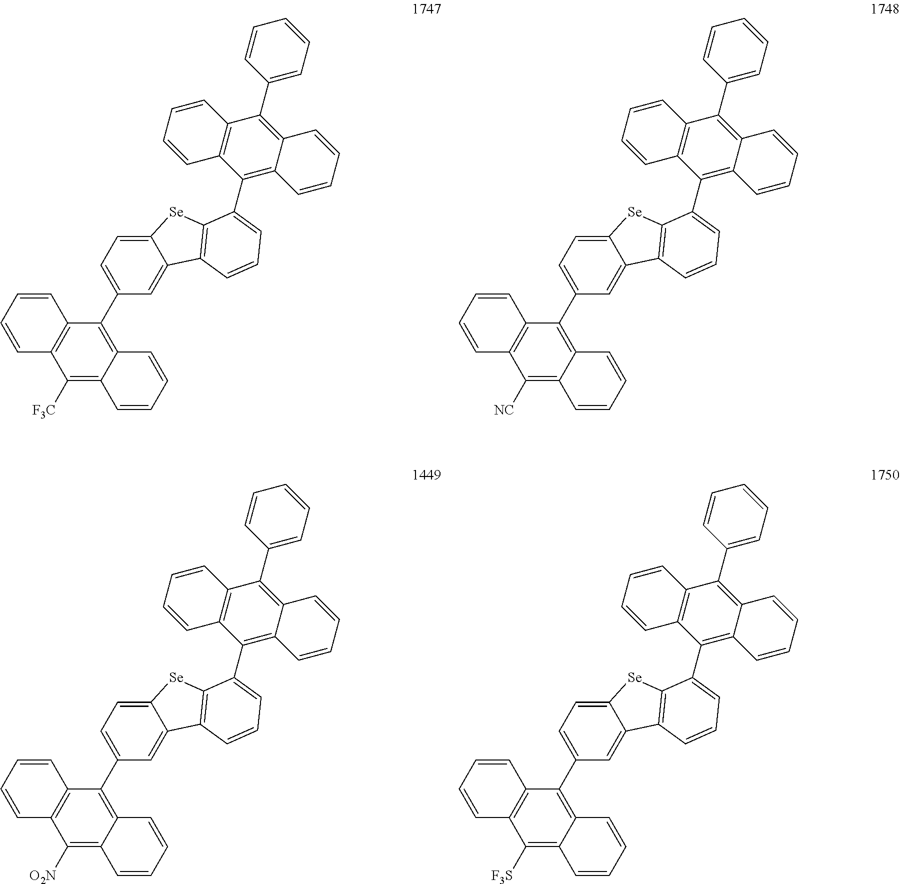

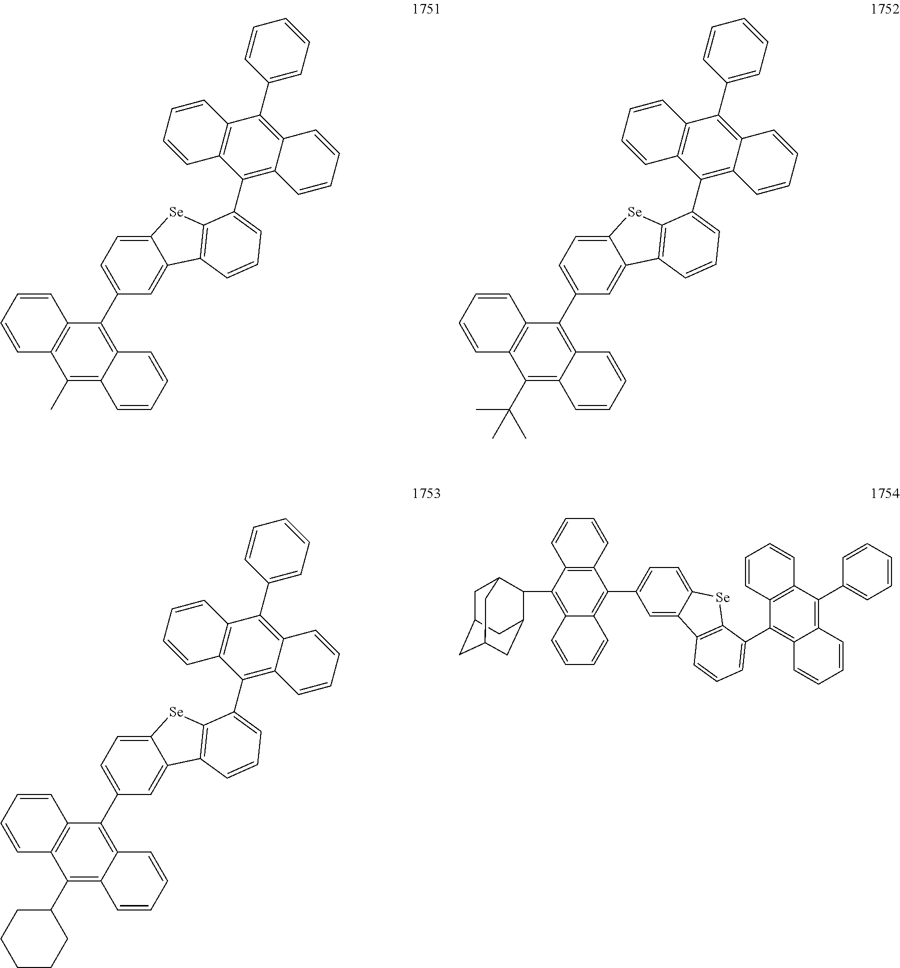 [SMILES: Cc1c2ccccc2c(-c2ccc3[se]c4c(-c5c6ccccc6c(-c6ccccc6)c6ccccc56)cccc4c3c2)c2ccccc12 ; CC(C)(C)c1c2ccccc2c(-c2ccc3[se]c4c(-c5c6ccccc6c(-c6ccccc6)c6ccccc56)cccc4c3c2)c2ccccc12 ; c1ccc(-c2c3ccccc3c(-c3cccc4c3[se]c3ccc(-c5c6ccccc6c(C6CCCCC6)c6ccccc56)cc34)c3ccccc23)cc1 ; c1ccc(-c2c3ccccc3c(-c3cccc4c3[se]c3ccc(-c5c6ccccc6c(C6C7CC8CC(C7)CC6C8)c6ccccc56)cc34)c3ccccc23)cc1]

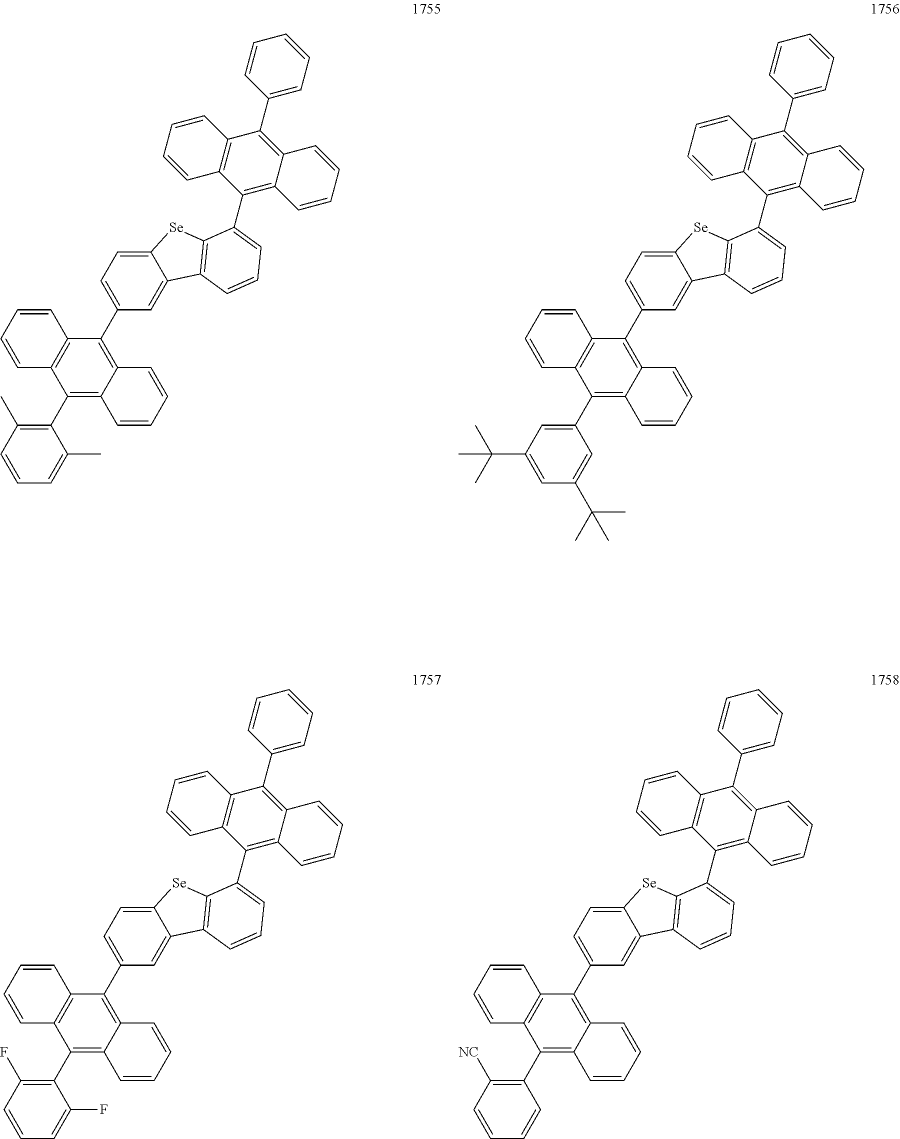 [SMILES: Cc1cccc(C)c1-c1c2ccccc2c(-c2ccc3[se]c4c(-c5c6ccccc6c(-c6ccccc6)c6ccccc56)cccc4c3c2)c2ccccc12 ; CC(C)(C)c1cc(-c2c3ccccc3c(-c3ccc4[se]c5c(-c6c7ccccc7c(-c7ccccc7)c7ccccc67)cccc5c4c3)c3ccccc23)cc(C(C)(C)C)c1 ; Fc1cccc(F)c1-c1c2ccccc2c(-c2ccc3[se]c4c(-c5c6ccccc6c(-c6ccccc6)c6ccccc56)cccc4c3c2)c2ccccc12 ; N#Cc1ccccc1-c1c2ccccc2c(-c2ccc3[se]c4c(-c5c6ccccc6c(-c6ccccc6)c6ccccc56)cccc4c3c2)c2ccccc12]

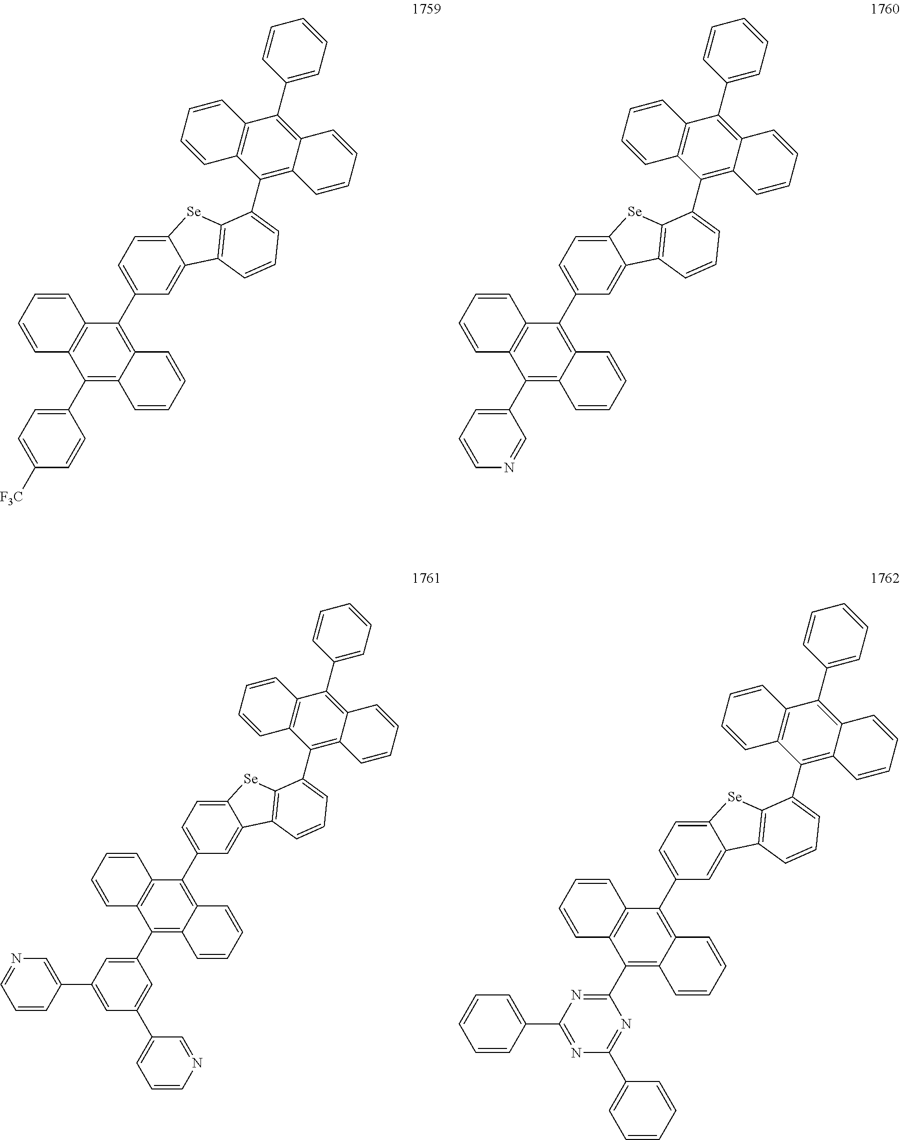 [SMILES: FC(F)(F)c1ccc(-c2c3ccccc3c(-c3ccc4[se]c5c(-c6c7ccccc7c(-c7ccccc7)c7ccccc67)cccc5c4c3)c3ccccc23)cc1 ; c1ccc(-c2c3ccccc3c(-c3cccc4c3[se]c3ccc(-c5c6ccccc6c(-c6cccnc6)c6ccccc56)cc34)c3ccccc23)cc1 ; c1ccc(-c2c3ccccc3c(-c3cccc4c3[se]c3ccc(-c5c6ccccc6c(-c6cc(-c7cccnc7)cc(-c7cccnc7)c6)c6ccccc56)cc34)c3ccccc23)cc1 ; c1ccc(-c2nc(-c3ccccc3)nc(-c3c4ccccc4c(-c4ccc5[se]c6c(-c7c8ccccc8c(-c8ccccc8)c8ccccc78)cccc6c5c4)c4ccccc34)n2)cc1]

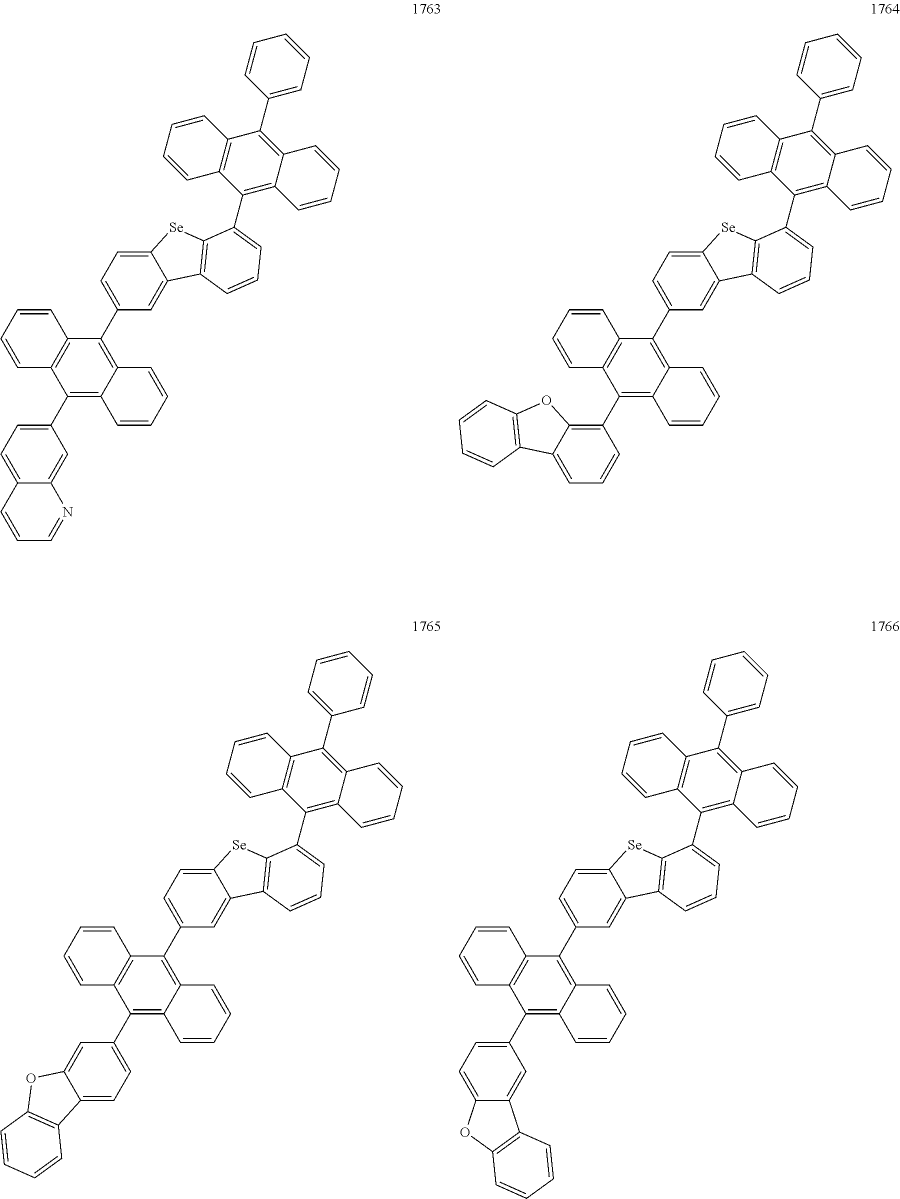 [SMILES: c1ccc(-c2c3ccccc3c(-c3cccc4c3[se]c3ccc(-c5c6ccccc6c(-c6ccc7cccnc7c6)c6ccccc56)cc34)c3ccccc23)cc1 ; c1ccc(-c2c3ccccc3c(-c3cccc4c3[se]c3ccc(-c5c6ccccc6c(-c6cccc7c6oc6ccccc67)c6ccccc56)cc34)c3ccccc23)cc1 ; c1ccc(-c2c3ccccc3c(-c3cccc4c3[se]c3ccc(-c5c6ccccc6c(-c6ccc7c(c6)oc6ccccc67)c6ccccc56)cc34)c3ccccc23)cc1 ; c1ccc(-c2c3ccccc3c(-c3cccc4c3[se]c3ccc(-c5c6ccccc6c(-c6ccc7oc8ccccc8c7c6)c6ccccc56)cc34)c3ccccc23)cc1]

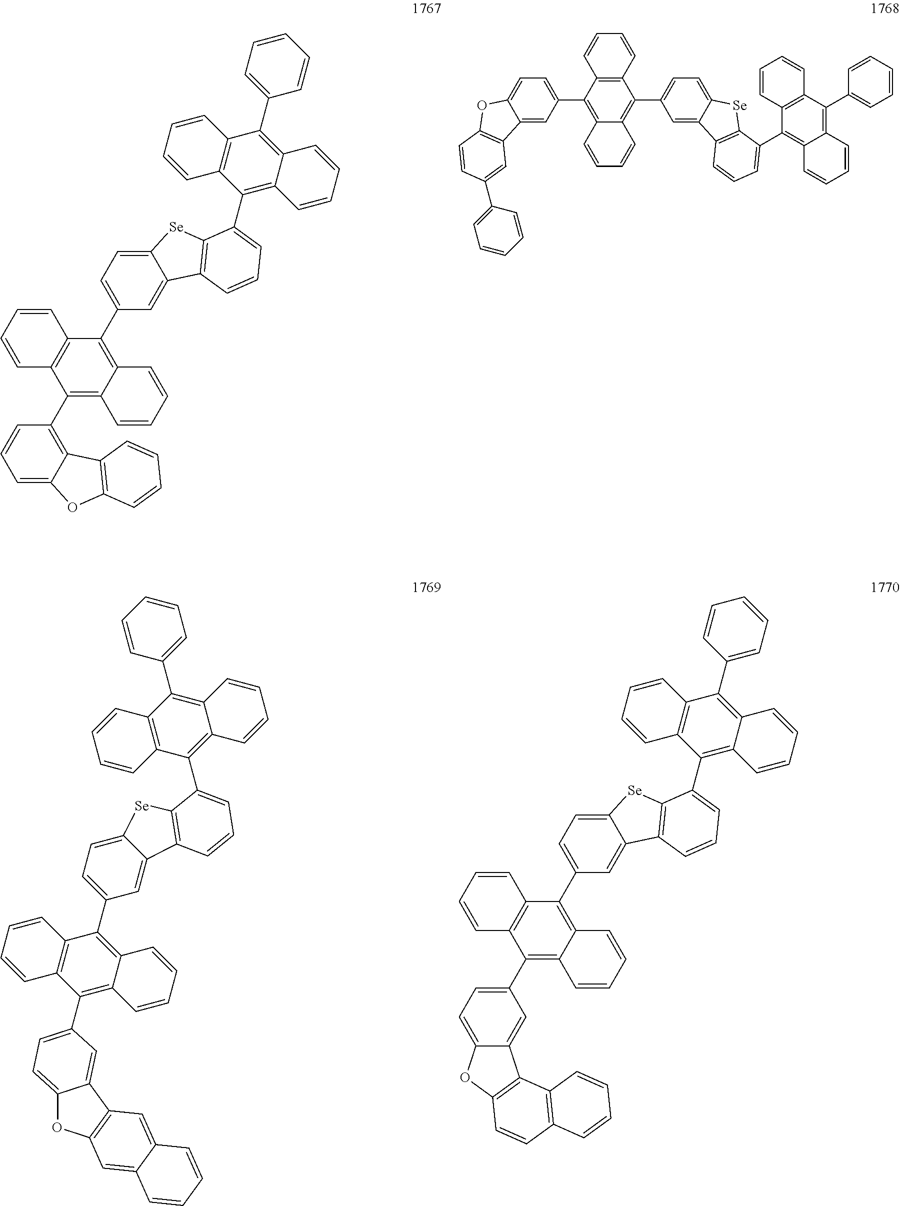 [SMILES: c1ccc(-c2c3ccccc3c(-c3cccc4c3[se]c3ccc(-c5c6ccccc6c(-c6cccc7oc8ccccc8c67)c6ccccc56)cc34)c3ccccc23)cc1 ; c1ccc(-c2ccc3oc4ccc(-c5c6ccccc6c(-c6ccc7[se]c8c(-c9c%10ccccc%10c(-c%10ccccc%10)c%10ccccc9%10)cccc8c7c6)c6ccccc56)cc4c3c2)cc1 ; c1ccc(-c2c3ccccc3c(-c3cccc4c3[se]c3ccc(-c5c6ccccc6c(-c6ccc7oc8cc9ccccc9cc8c7c6)c6ccccc56)cc34)c3ccccc23)cc1 ; c1ccc(-c2c3ccccc3c(-c3cccc4c3[se]c3ccc(-c5c6ccccc6c(-c6ccc7oc8ccc9ccccc9c8c7c6)c6ccccc56)cc34)c3ccccc23)cc1]

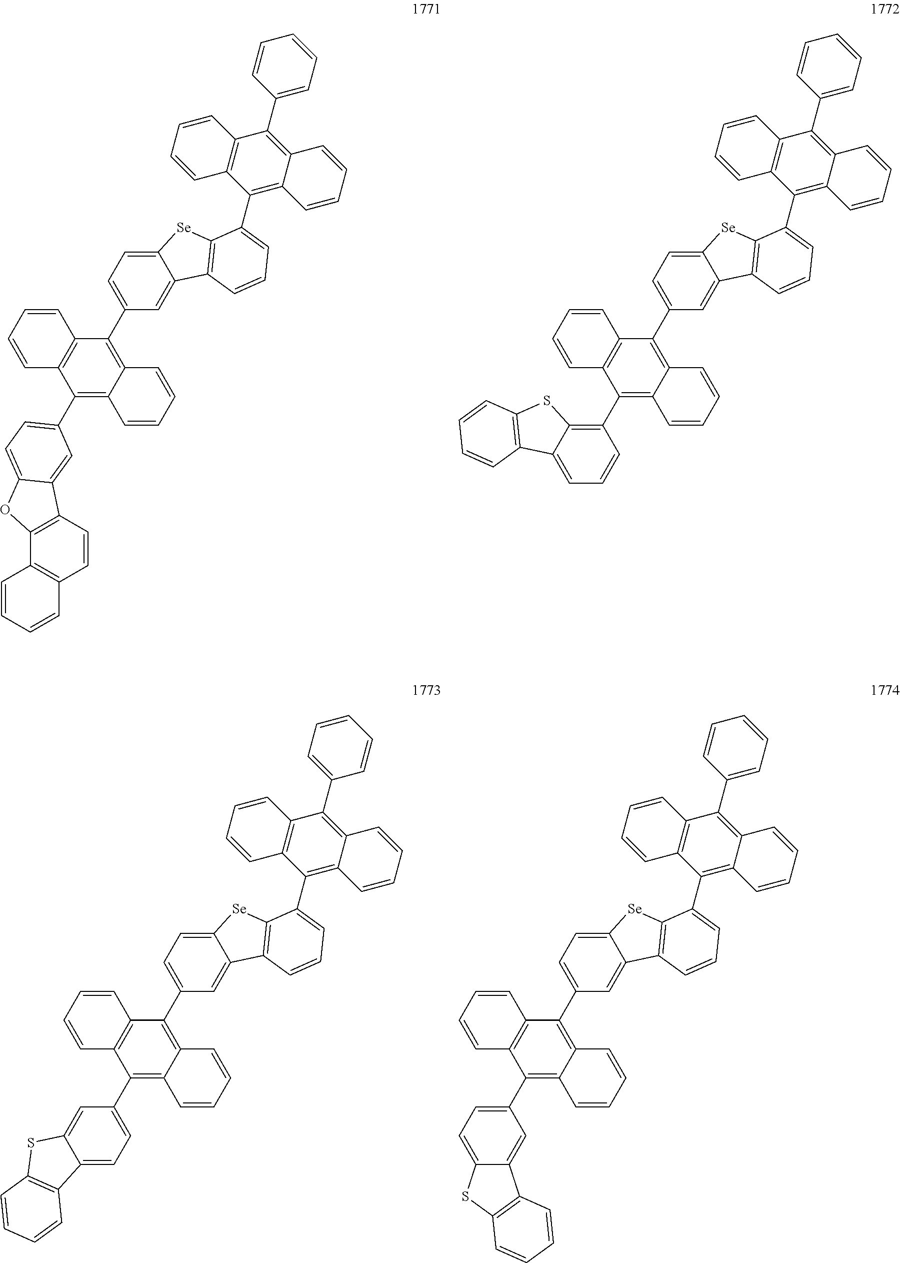 [SMILES: c1ccc(-c2c3ccccc3c(-c3cccc4c3[se]c3ccc(-c5c6ccccc6c(-c6ccc7oc8c9ccccc9ccc8c7c6)c6ccccc56)cc34)c3ccccc23)cc1 ; c1ccc(-c2c3ccccc3c(-c3cccc4c3[se]c3ccc(-c5c6ccccc6c(-c6cccc7c6sc6ccccc67)c6ccccc56)cc34)c3ccccc23)cc1 ; c1ccc(-c2c3ccccc3c(-c3cccc4c3[se]c3ccc(-c5c6ccccc6c(-c6ccc7c(c6)sc6ccccc67)c6ccccc56)cc34)c3ccccc23)cc1 ; c1ccc(-c2c3ccccc3c(-c3cccc4c3[se]c3ccc(-c5c6ccccc6c(-c6ccc7sc8ccccc8c7c6)c6ccccc56)cc34)c3ccccc23)cc1]

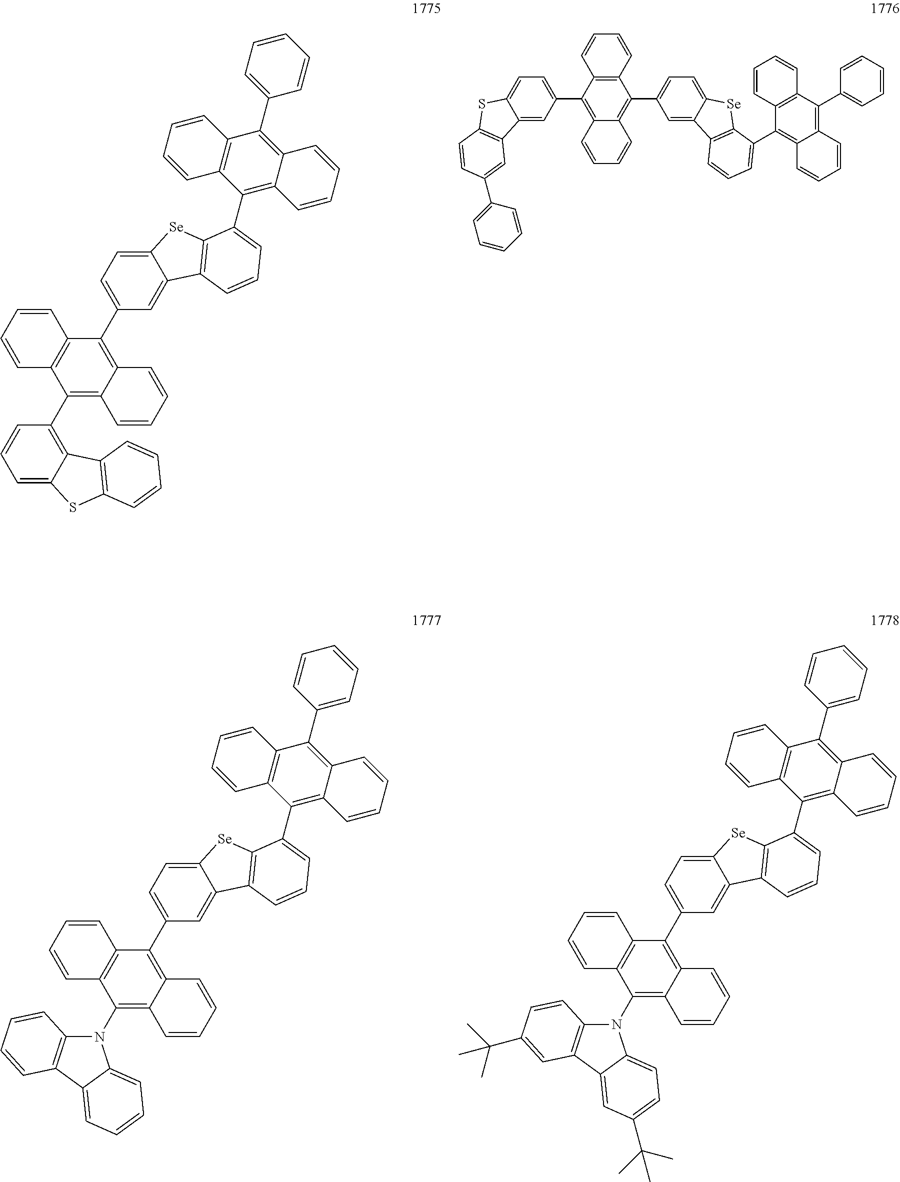 [SMILES: c1ccc(-c2c3ccccc3c(-c3cccc4c3[se]c3ccc(-c5c6ccccc6c(-c6cccc7sc8ccccc8c67)c6ccccc56)cc34)c3ccccc23)cc1 ; c1ccc(-c2ccc3sc4ccc(-c5c6ccccc6c(-c6ccc7[se]c8c(-c9c%10ccccc%10c(-c%10ccccc%10)c%10ccccc9%10)cccc8c7c6)c6ccccc56)cc4c3c2)cc1 ; c1ccc(-c2c3ccccc3c(-c3cccc4c3[se]c3ccc(-c5c6ccccc6c(-n6c7ccccc7c7ccccc76)c6ccccc56)cc34)c3ccccc23)cc1 ; CC(C)(C)c1ccc2c(c1)c1cc(C(C)(C)C)ccc1n2-c1c2ccccc2c(-c2ccc3[se]c4c(-c5c6ccccc6c(-c6ccccc6)c6ccccc56)cccc4c3c2)c2ccccc12]

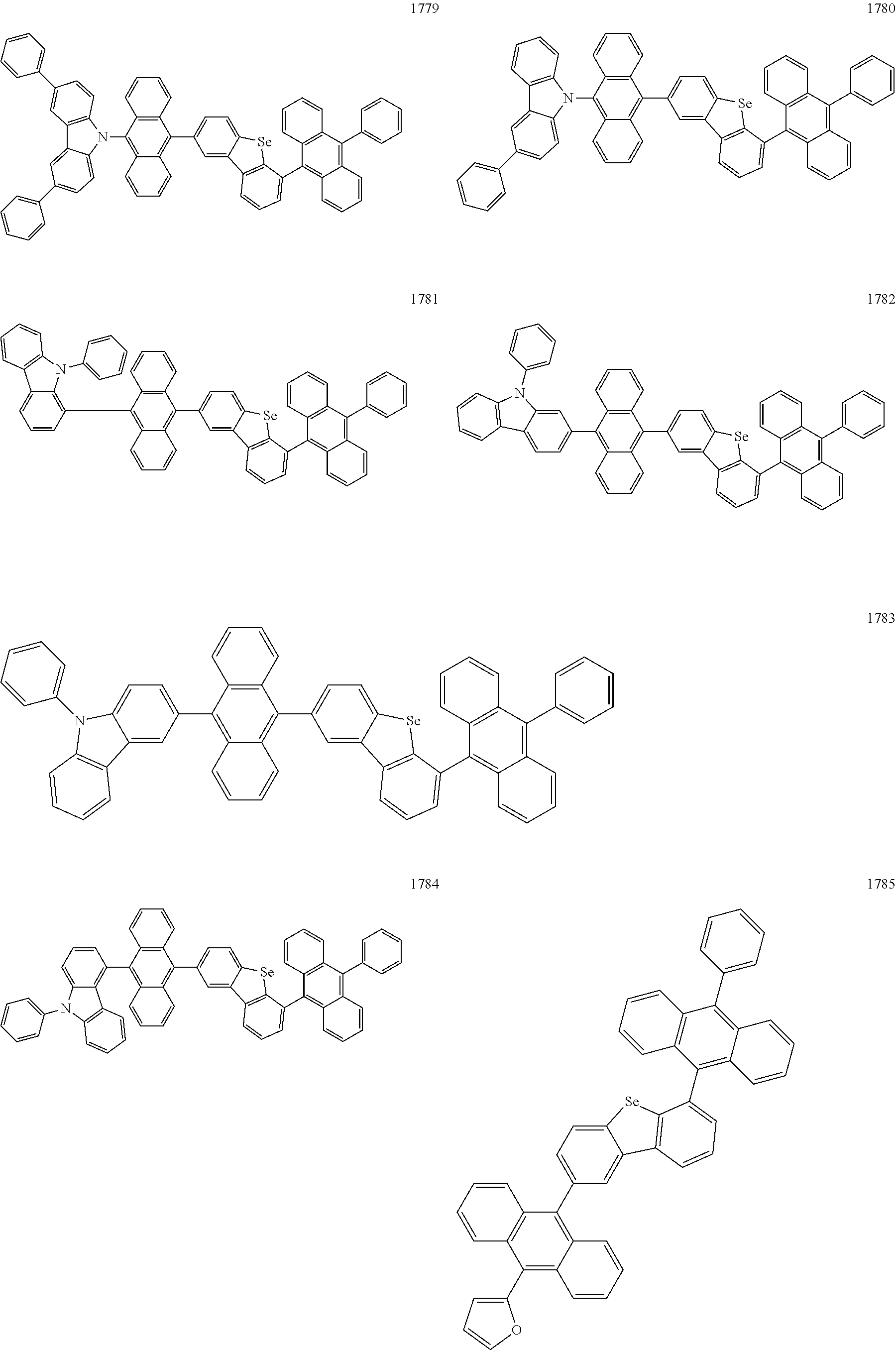

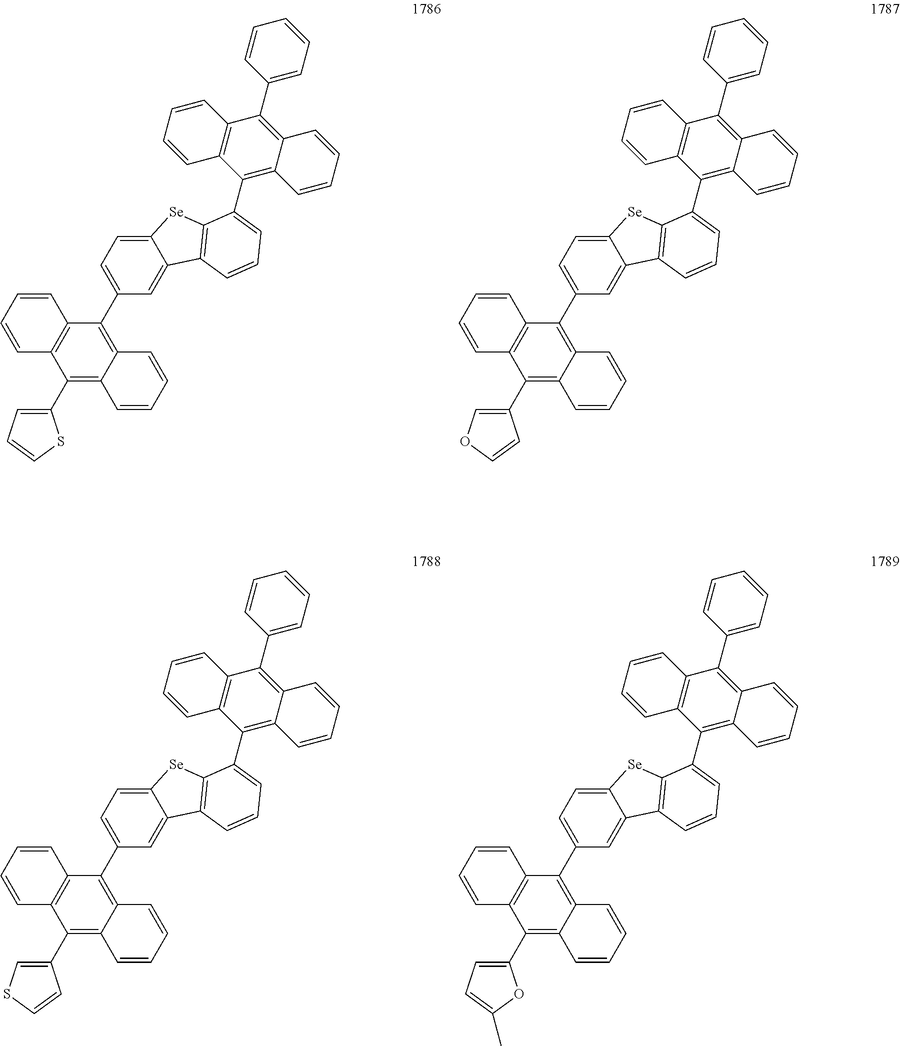 [SMILES: c1ccc(-c2c3ccccc3c(-c3cccc4c3[se]c3ccc(-c5c6ccccc6c(-c6cccs6)c6ccccc56)cc34)c3ccccc23)cc1 ; c1ccc(-c2c3ccccc3c(-c3cccc4c3[se]c3ccc(-c5c6ccccc6c(-c6ccoc6)c6ccccc56)cc34)c3ccccc23)cc1 ; c1ccc(-c2c3ccccc3c(-c3cccc4c3[se]c3ccc(-c5c6ccccc6c(-c6ccsc6)c6ccccc56)cc34)c3ccccc23)cc1 ; Cc1ccc(-c2c3ccccc3c(-c3ccc4[se]c5c(-c6c7ccccc7c(-c7ccccc7)c7ccccc67)cccc5c4c3)c3ccccc23)o1]

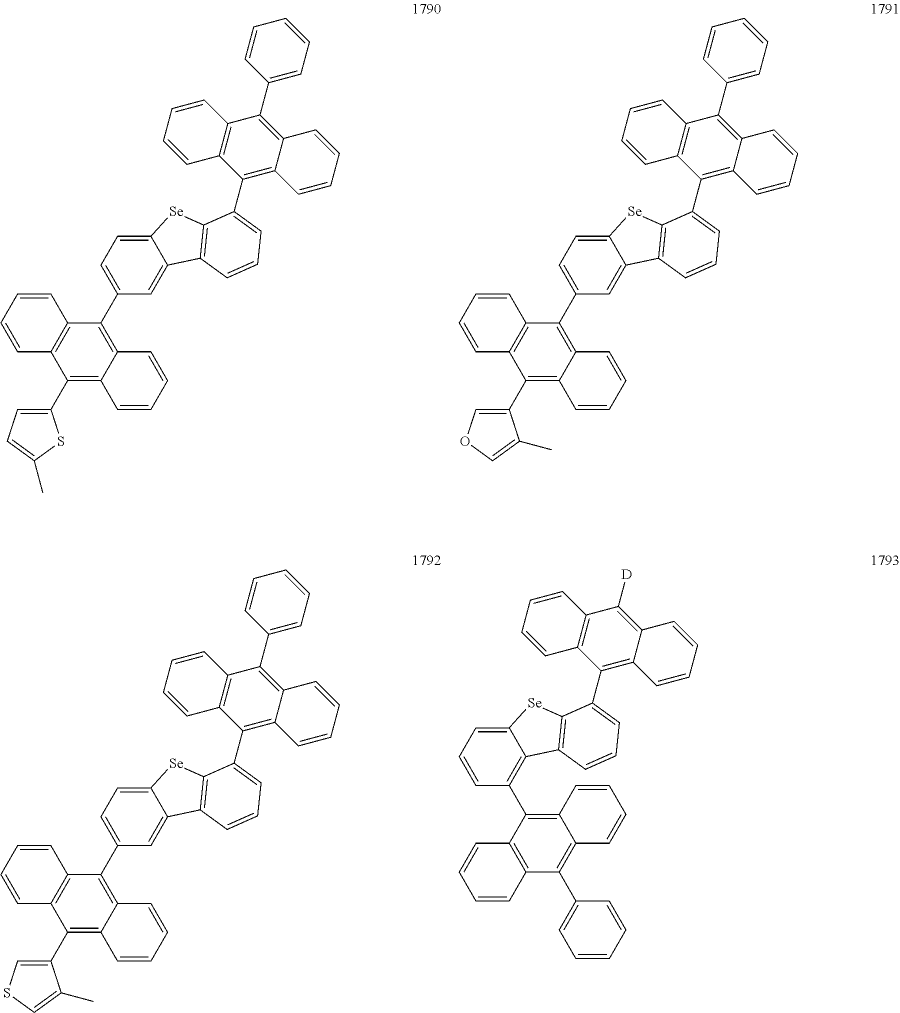 [SMILES: Cc1ccc(-c2c3ccccc3c(-c3ccc4[se]c5c(-c6c7ccccc7c(-c7ccccc7)c7ccccc67)cccc5c4c3)c3ccccc23)s1 ; Cc1cocc1-c1c2ccccc2c(-c2ccc3[se]c4c(-c5c6ccccc6c(-c6ccccc6)c6ccccc56)cccc4c3c2)c2ccccc12 ; Cc1cscc1-c1c2ccccc2c(-c2ccc3[se]c4c(-c5c6ccccc6c(-c6ccccc6)c6ccccc56)cccc4c3c2)c2ccccc12 ; [2H]c1c2ccccc2c(-c2cccc3c2[se]c2cccc(-c4c5ccccc5c(-c5ccccc5)c5ccccc45)c23)c2ccccc12]

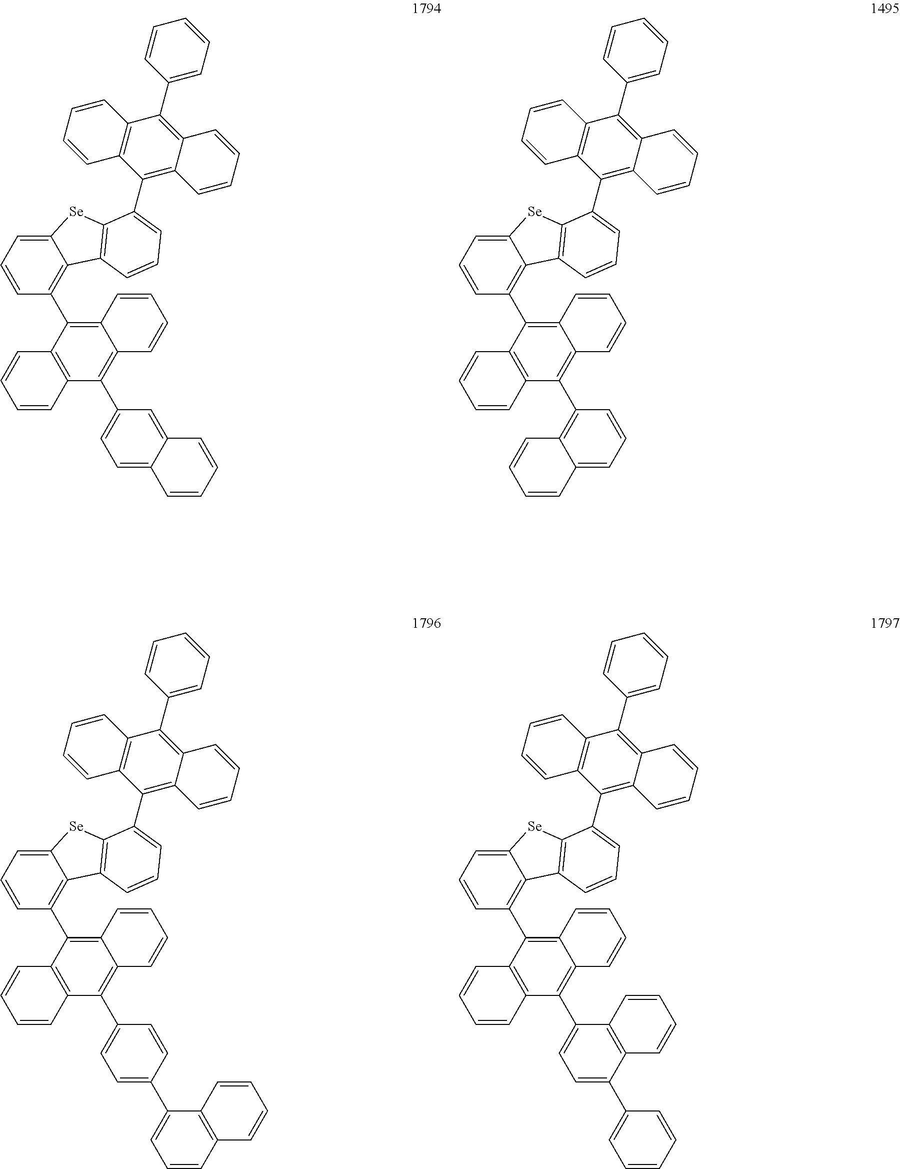 [SMILES: c1ccc(-c2c3ccccc3c(-c3cccc4c3[se]c3cccc(-c5c6ccccc6c(-c6ccc7ccccc7c6)c6ccccc56)c34)c3ccccc23)cc1 ; c1ccc(-c2c3ccccc3c(-c3cccc4c3[se]c3cccc(-c5c6ccccc6c(-c6cccc7ccccc67)c6ccccc56)c34)c3ccccc23)cc1 ; c1ccc(-c2c3ccccc3c(-c3cccc4c3[se]c3cccc(-c5c6ccccc6c(-c6ccc(-c7cccc8ccccc78)cc6)c6ccccc56)c34)c3ccccc23)cc1 ; c1ccc(-c2ccc(-c3c4ccccc4c(-c4cccc5[se]c6c(-c7c8ccccc8c(-c8ccccc8)c8ccccc78)cccc6c45)c4ccccc34)c3ccccc23)cc1]

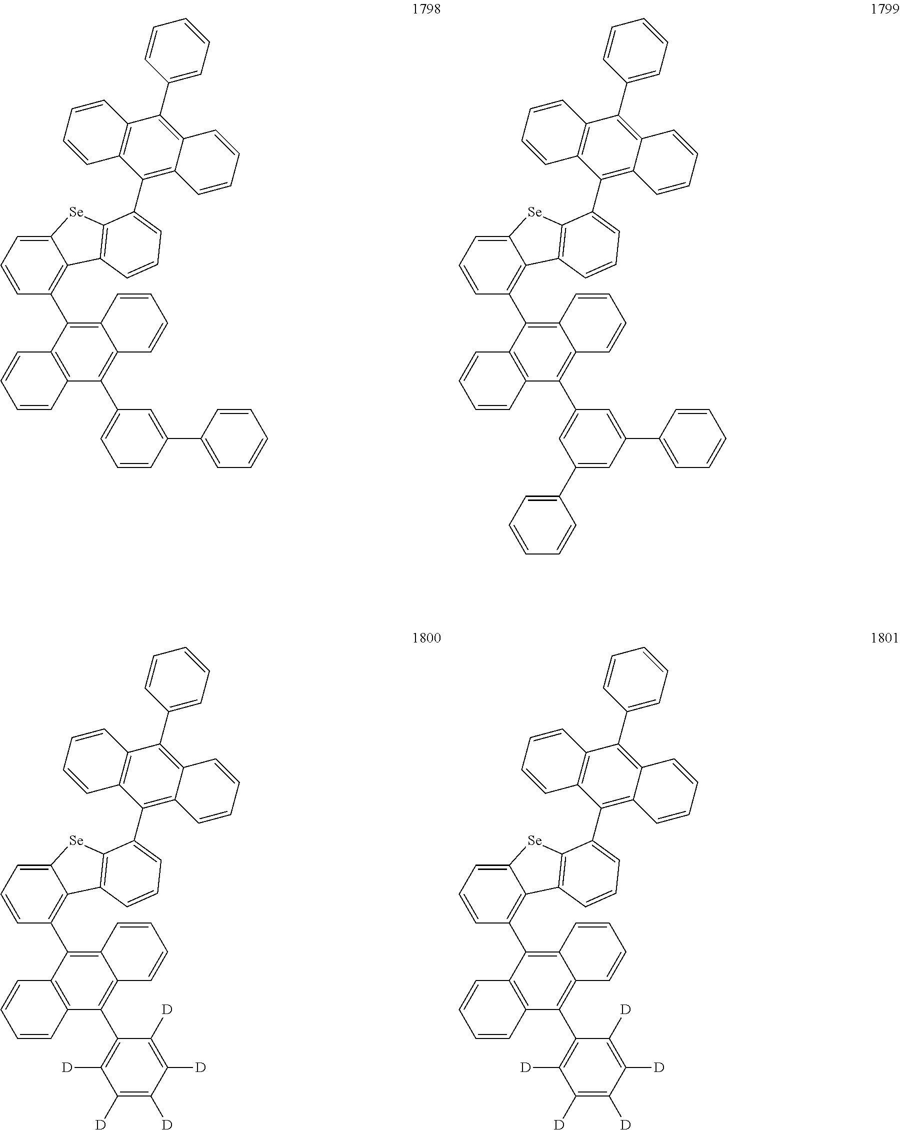 [SMILES: c1ccc(-c2cccc(-c3c4ccccc4c(-c4cccc5[se]c6c(-c7c8ccccc8c(-c8ccccc8)c8ccccc78)cccc6c45)c4ccccc34)c2)cc1 ; c1ccc(-c2cc(-c3ccccc3)cc(-c3c4ccccc4c(-c4cccc5[se]c6c(-c7c8ccccc8c(-c8ccccc8)c8ccccc78)cccc6c45)c4ccccc34)c2)cc1 ; [2H]c1c([2H])c([2H])c(-c2c3ccccc3c(-c3cccc4[se]c5c(-c6c7ccccc7c(-c7ccccc7)c7ccccc67)cccc5c34)c3ccccc23)c([2H])c1[2H] ; [2H]c1c([2H])c([2H])c(-c2c3ccccc3c(-c3cccc4[se]c5c(-c6c7ccccc7c(-c7ccccc7)c7ccccc67)cccc5c34)c3ccccc23)c([2H])c1[2H]]

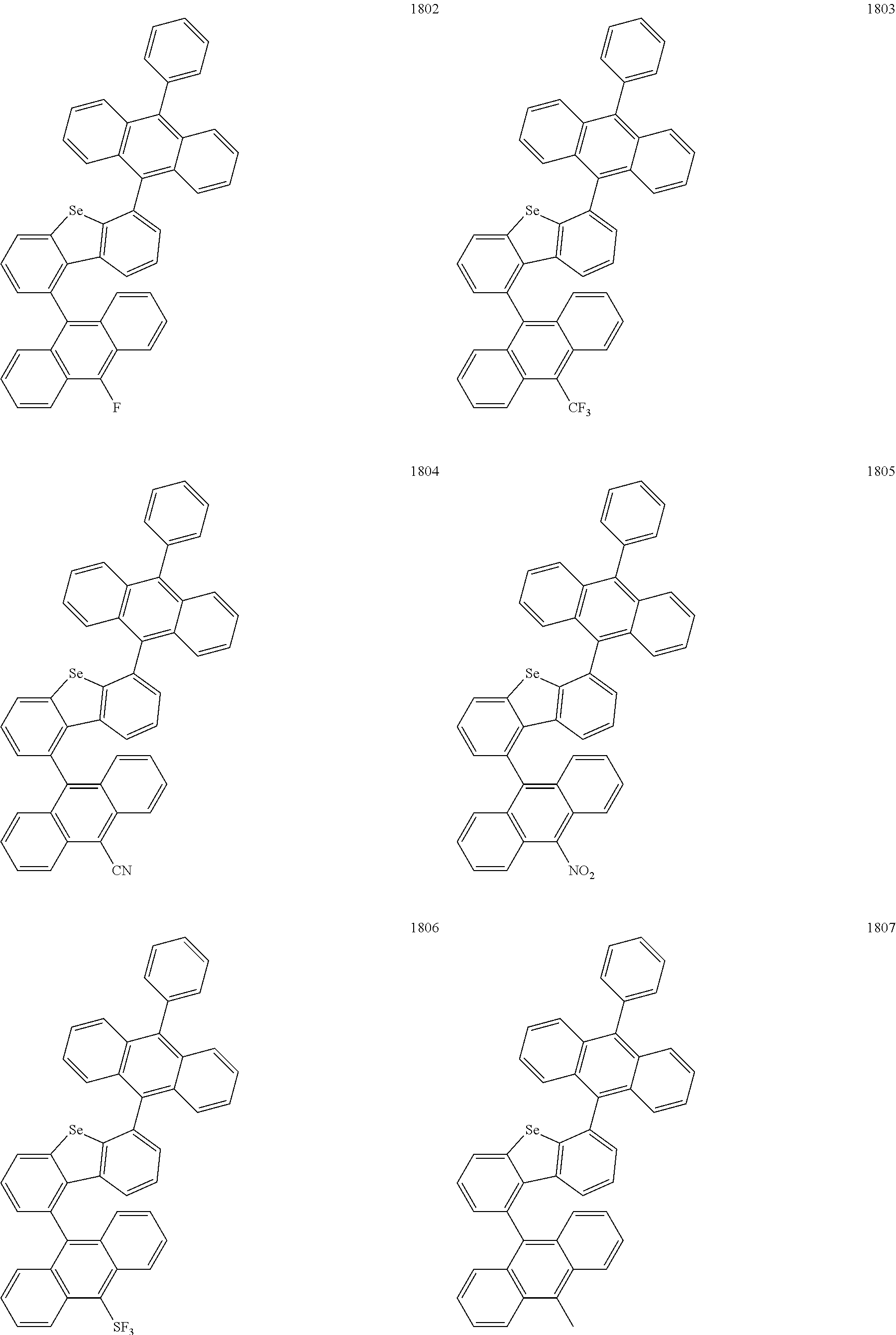

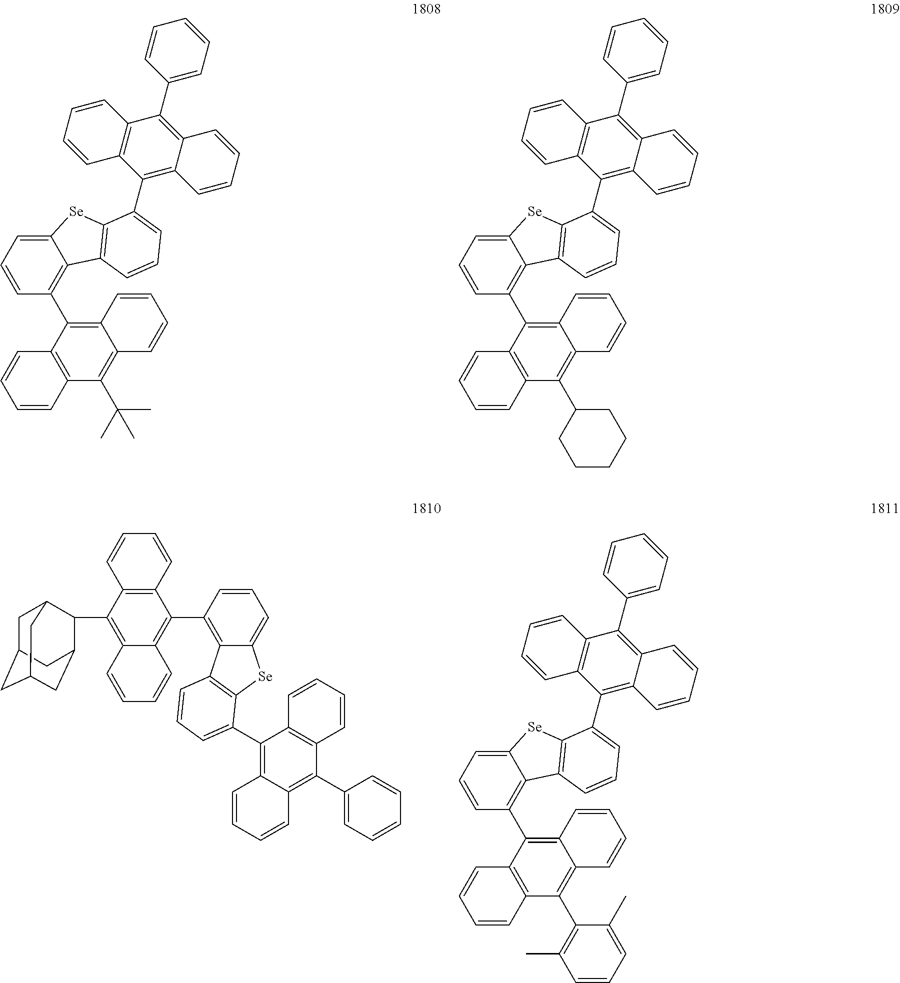 [SMILES: CC(C)(C)c1c2ccccc2c(-c2cccc3[se]c4c(-c5c6ccccc6c(-c6ccccc6)c6ccccc56)cccc4c23)c2ccccc12 ; c1ccc(-c2c3ccccc3c(-c3cccc4c3[se]c3cccc(-c5c6ccccc6c(C6CCCCC6)c6ccccc56)c34)c3ccccc23)cc1 ; c1ccc(-c2c3ccccc3c(-c3cccc4c3[se]c3cccc(-c5c6ccccc6c(C6C7CC8CC(C7)CC6C8)c6ccccc56)c34)c3ccccc23)cc1 ; Cc1cccc(C)c1-c1c2ccccc2c(-c2cccc3[se]c4c(-c5c6ccccc6c(-c6ccccc6)c6ccccc56)cccc4c23)c2ccccc12]

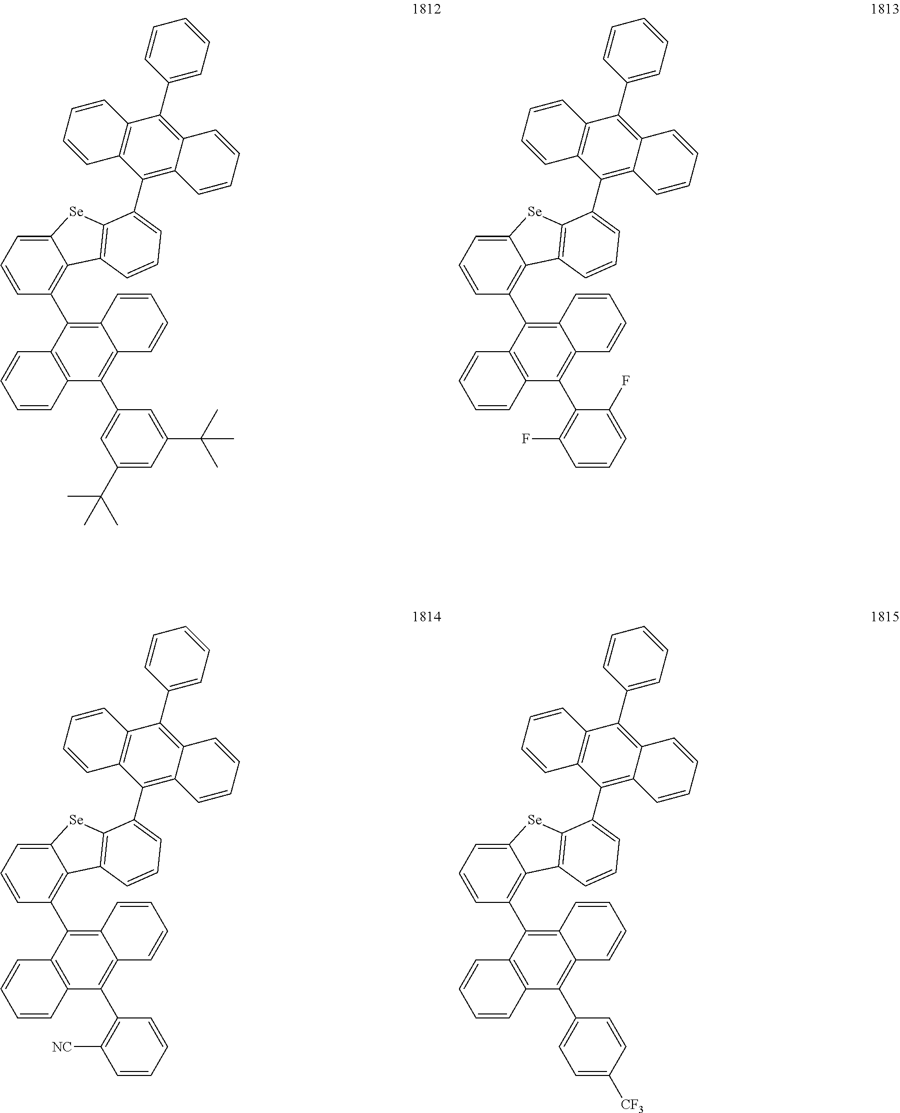 [SMILES: CC(C)(C)c1cc(-c2c3ccccc3c(-c3cccc4[se]c5c(-c6c7ccccc7c(-c7ccccc7)c7ccccc67)cccc5c34)c3ccccc23)cc(C(C)(C)C)c1 ; Fc1cccc(F)c1-c1c2ccccc2c(-c2cccc3[se]c4c(-c5c6ccccc6c(-c6ccccc6)c6ccccc56)cccc4c23)c2ccccc12 ; N#Cc1ccccc1-c1c2ccccc2c(-c2cccc3[se]c4c(-c5c6ccccc6c(-c6ccccc6)c6ccccc56)cccc4c23)c2ccccc12 ; FC(F)(F)c1ccc(-c2c3ccccc3c(-c3cccc4[se]c5c(-c6c7ccccc7c(-c7ccccc7)c7ccccc67)cccc5c34)c3ccccc23)cc1]

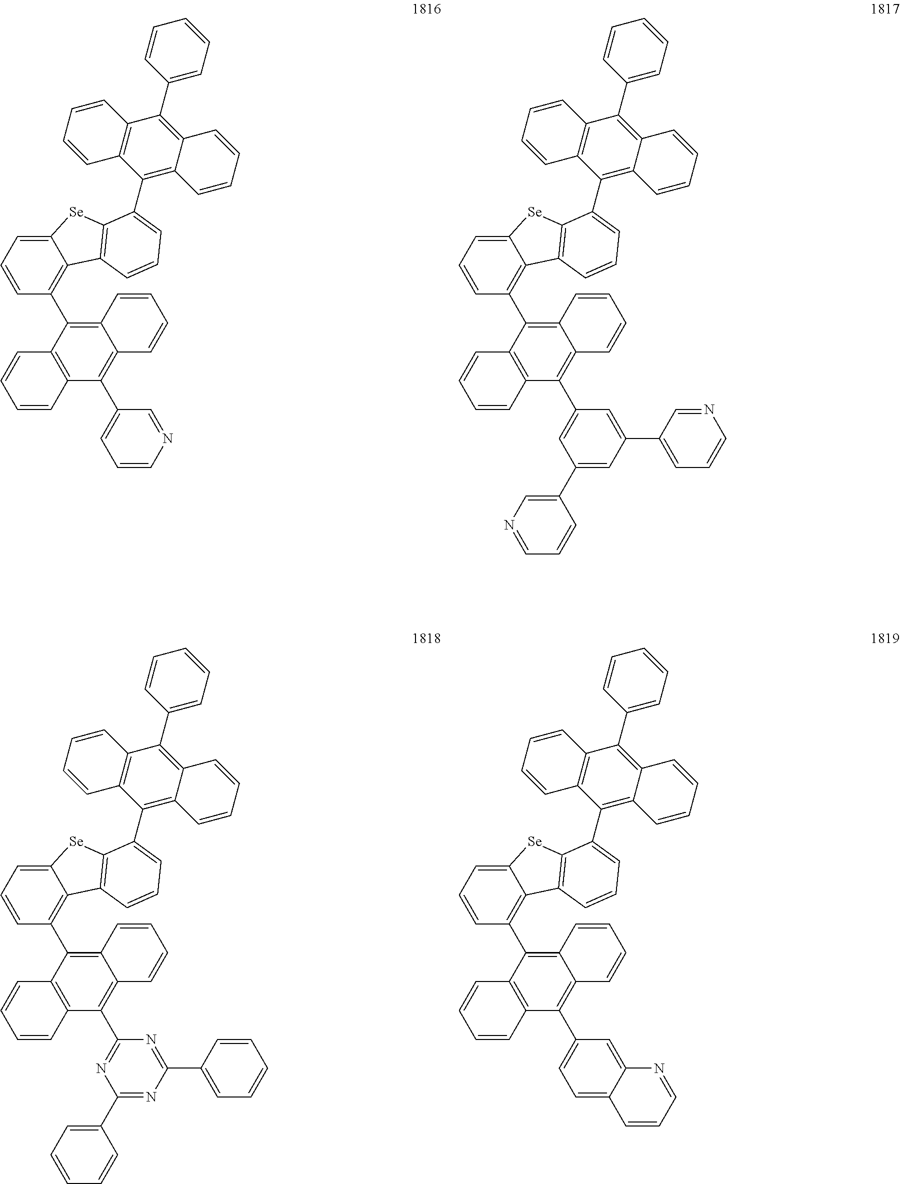 [SMILES: c1ccc(-c2c3ccccc3c(-c3cccc4c3[se]c3cccc(-c5c6ccccc6c(-c6cccnc6)c6ccccc56)c34)c3ccccc23)cc1 ; c1ccc(-c2c3ccccc3c(-c3cccc4c3[se]c3cccc(-c5c6ccccc6c(-c6cc(-c7cccnc7)cc(-c7cccnc7)c6)c6ccccc56)c34)c3ccccc23)cc1 ; c1ccc(-c2nc(-c3ccccc3)nc(-c3c4ccccc4c(-c4cccc5[se]c6c(-c7c8ccccc8c(-c8ccccc8)c8ccccc78)cccc6c45)c4ccccc34)n2)cc1 ; c1ccc(-c2c3ccccc3c(-c3cccc4c3[se]c3cccc(-c5c6ccccc6c(-c6ccc7cccnc7c6)c6ccccc56)c34)c3ccccc23)cc1]

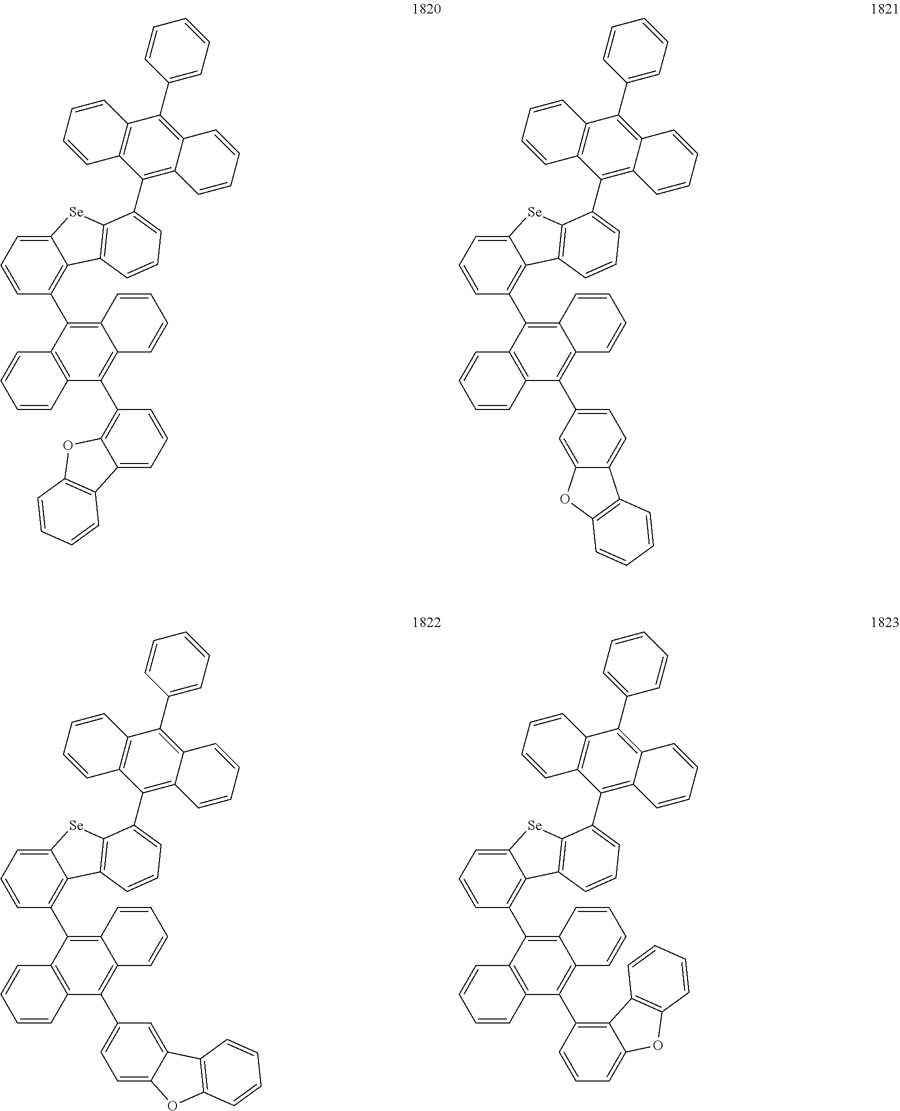 [SMILES: c1ccc(-c2c3ccccc3c(-c3cccc4c3[se]c3cccc(-c5c6ccccc6c(-c6cccc7c6oc6ccccc67)c6ccccc56)c34)c3ccccc23)cc1 ; c1ccc(-c2c3ccccc3c(-c3cccc4c3[se]c3cccc(-c5c6ccccc6c(-c6ccc7c(c6)oc6ccccc67)c6ccccc56)c34)c3ccccc23)cc1 ; c1ccc(-c2c3ccccc3c(-c3cccc4c3[se]c3cccc(-c5c6ccccc6c(-c6ccc7oc8ccccc8c7c6)c6ccccc56)c34)c3ccccc23)cc1 ; c1ccc(-c2c3ccccc3c(-c3cccc4c3[se]c3cccc(-c5c6ccccc6c(-c6cccc7oc8ccccc8c67)c6ccccc56)c34)c3ccccc23)cc1]

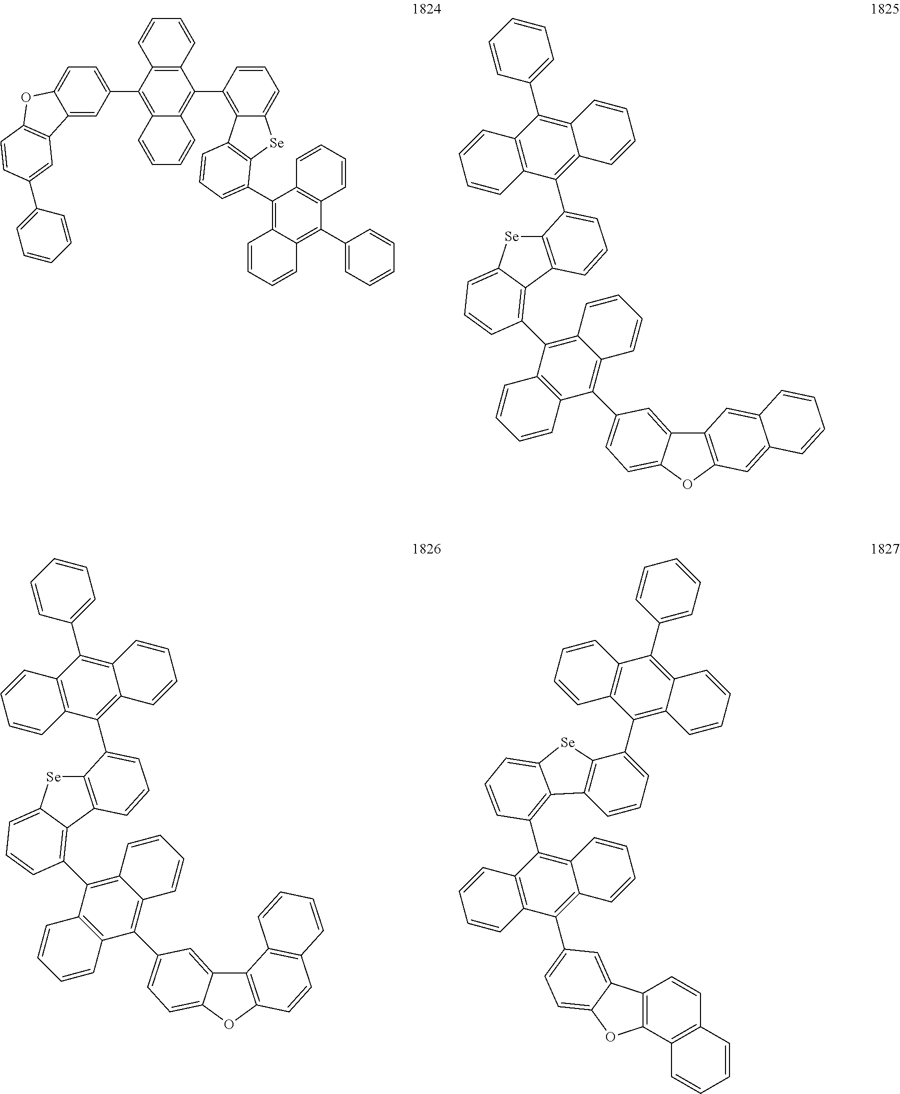 [SMILES: c1ccc(-c2ccc3oc4ccc(-c5c6ccccc6c(-c6cccc7[se]c8c(-c9c%10ccccc%10c(-c%10ccccc%10)c%10ccccc9%10)cccc8c67)c6ccccc56)cc4c3c2)cc1 ; c1ccc(-c2c3ccccc3c(-c3cccc4c3[se]c3cccc(-c5c6ccccc6c(-c6ccc7oc8cc9ccccc9cc8c7c6)c6ccccc56)c34)c3ccccc23)cc1 ; c1ccc(-c2c3ccccc3c(-c3cccc4c3[se]c3cccc(-c5c6ccccc6c(-c6ccc7oc8ccc9ccccc9c8c7c6)c6ccccc56)c34)c3ccccc23)cc1 ; c1ccc(-c2c3ccccc3c(-c3cccc4c3[se]c3cccc(-c5c6ccccc6c(-c6ccc7oc8c9ccccc9ccc8c7c6)c6ccccc56)c34)c3ccccc23)cc1]

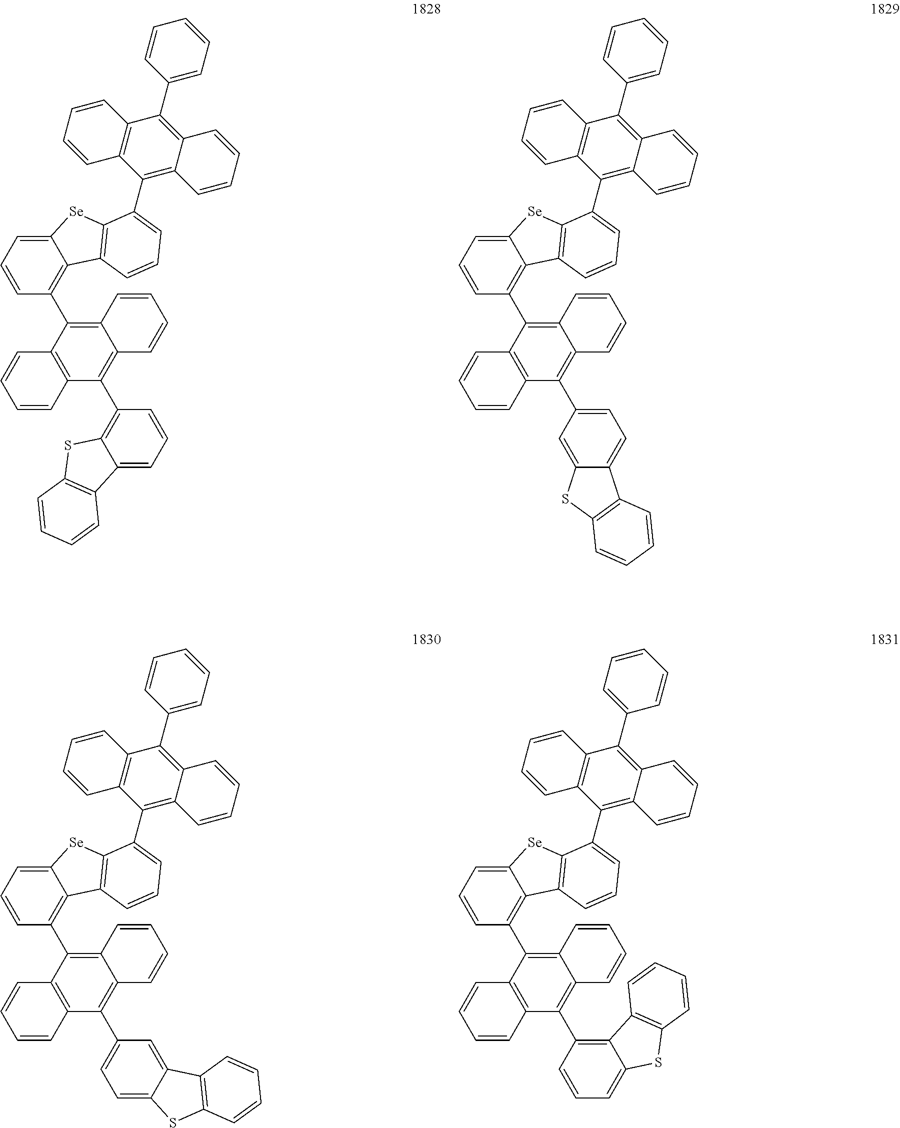 [SMILES: c1ccc(-c2c3ccccc3c(-c3cccc4c3[se]c3cccc(-c5c6ccccc6c(-c6cccc7c6sc6ccccc67)c6ccccc56)c34)c3ccccc23)cc1 ; c1ccc(-c2c3ccccc3c(-c3cccc4c3[se]c3cccc(-c5c6ccccc6c(-c6ccc7c(c6)sc6ccccc67)c6ccccc56)c34)c3ccccc23)cc1 ; c1ccc(-c2c3ccccc3c(-c3cccc4c3[se]c3cccc(-c5c6ccccc6c(-c6ccc7sc8ccccc8c7c6)c6ccccc56)c34)c3ccccc23)cc1 ; c1ccc(-c2c3ccccc3c(-c3cccc4c3[se]c3cccc(-c5c6ccccc6c(-c6cccc7sc8ccccc8c67)c6ccccc56)c34)c3ccccc23)cc1]

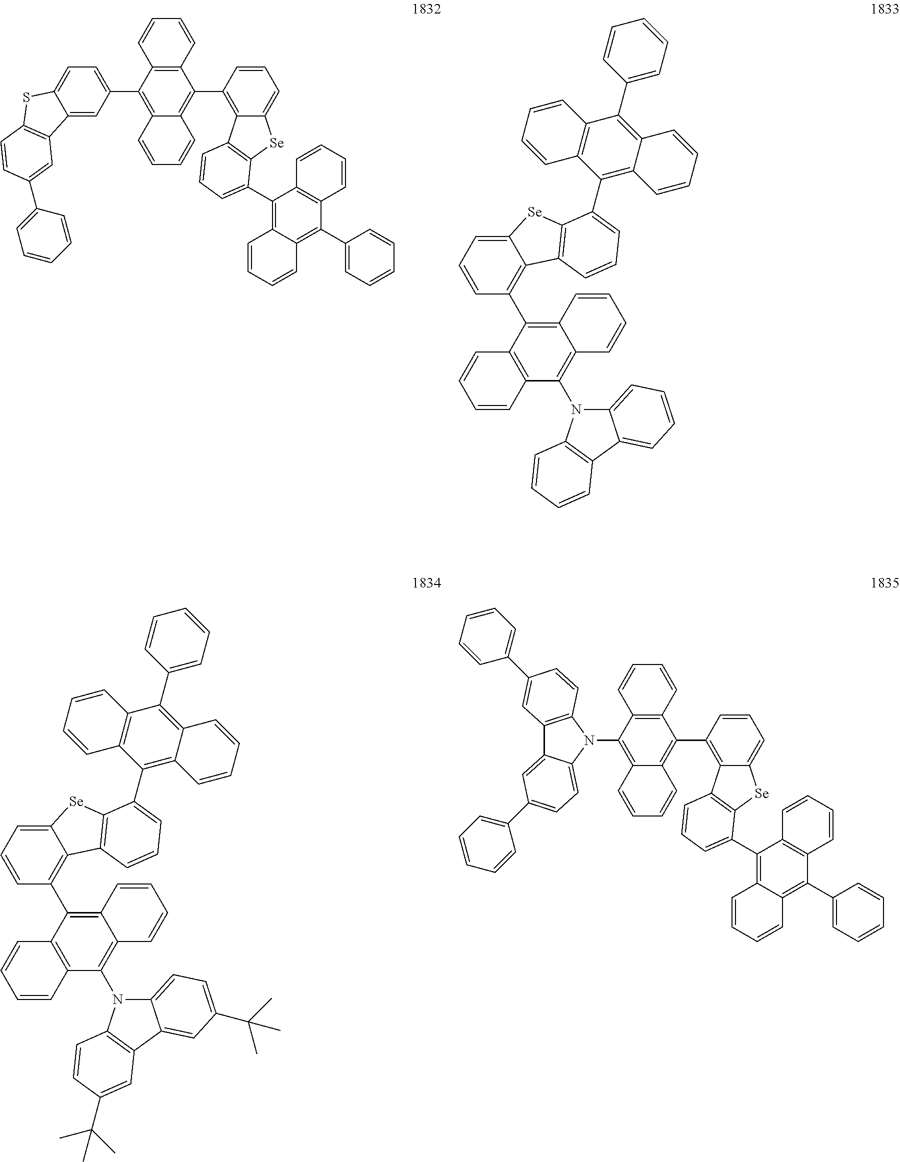 [SMILES: c1ccc(-c2ccc3sc4ccc(-c5c6ccccc6c(-c6cccc7[se]c8c(-c9c%10ccccc%10c(-c%10ccccc%10)c%10ccccc9%10)cccc8c67)c6ccccc56)cc4c3c2)cc1 ; c1ccc(-c2c3ccccc3c(-c3cccc4c3[se]c3cccc(-c5c6ccccc6c(-n6c7ccccc7c7ccccc76)c6ccccc56)c34)c3ccccc23)cc1 ; CC(C)(C)c1ccc2c(c1)c1cc(C(C)(C)C)ccc1n2-c1c2ccccc2c(-c2cccc3[se]c4c(-c5c6ccccc6c(-c6ccccc6)c6ccccc56)cccc4c23)c2ccccc12 ; c1ccc(-c2ccc3c(c2)c2cc(-c4ccccc4)ccc2n3-c2c3ccccc3c(-c3cccc4[se]c5c(-c6c7ccccc7c(-c7ccccc7)c7ccccc67)cccc5c34)c3ccccc23)cc1]

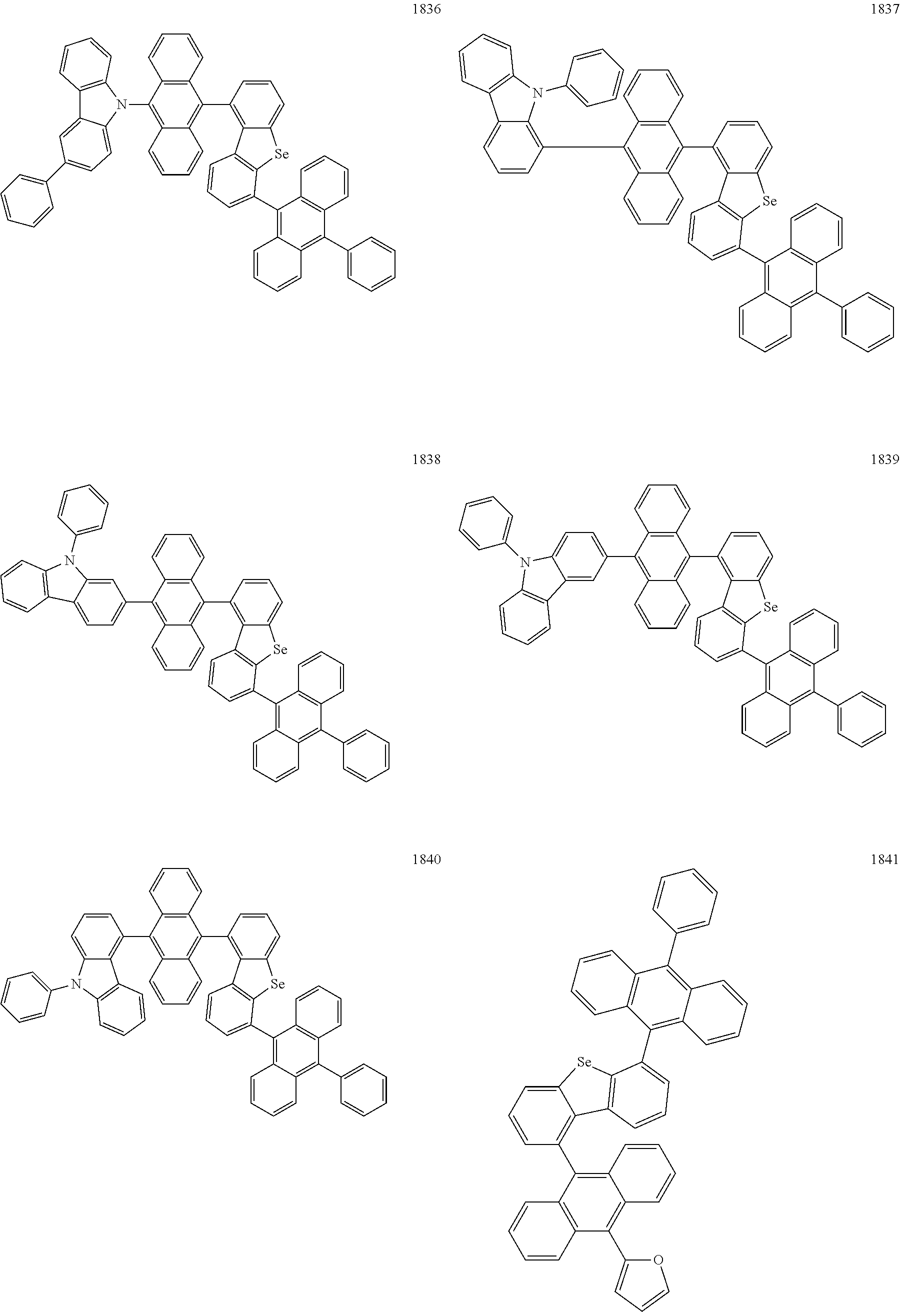

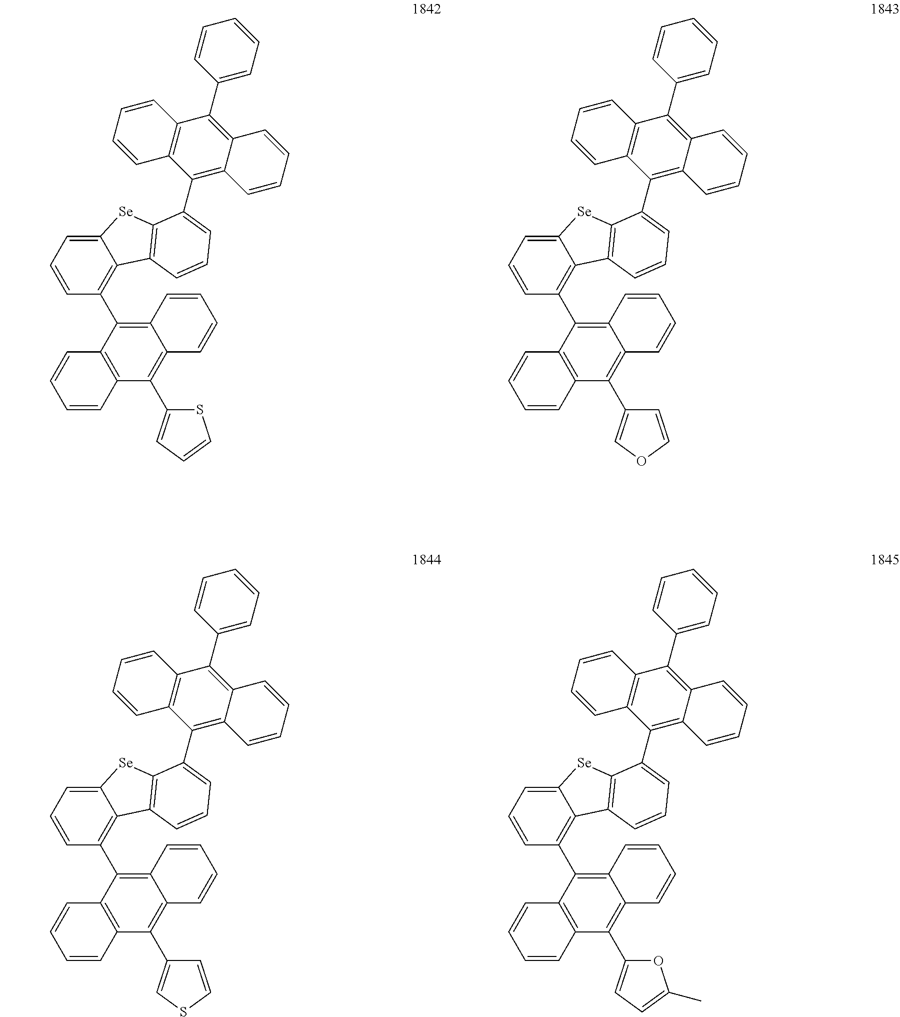

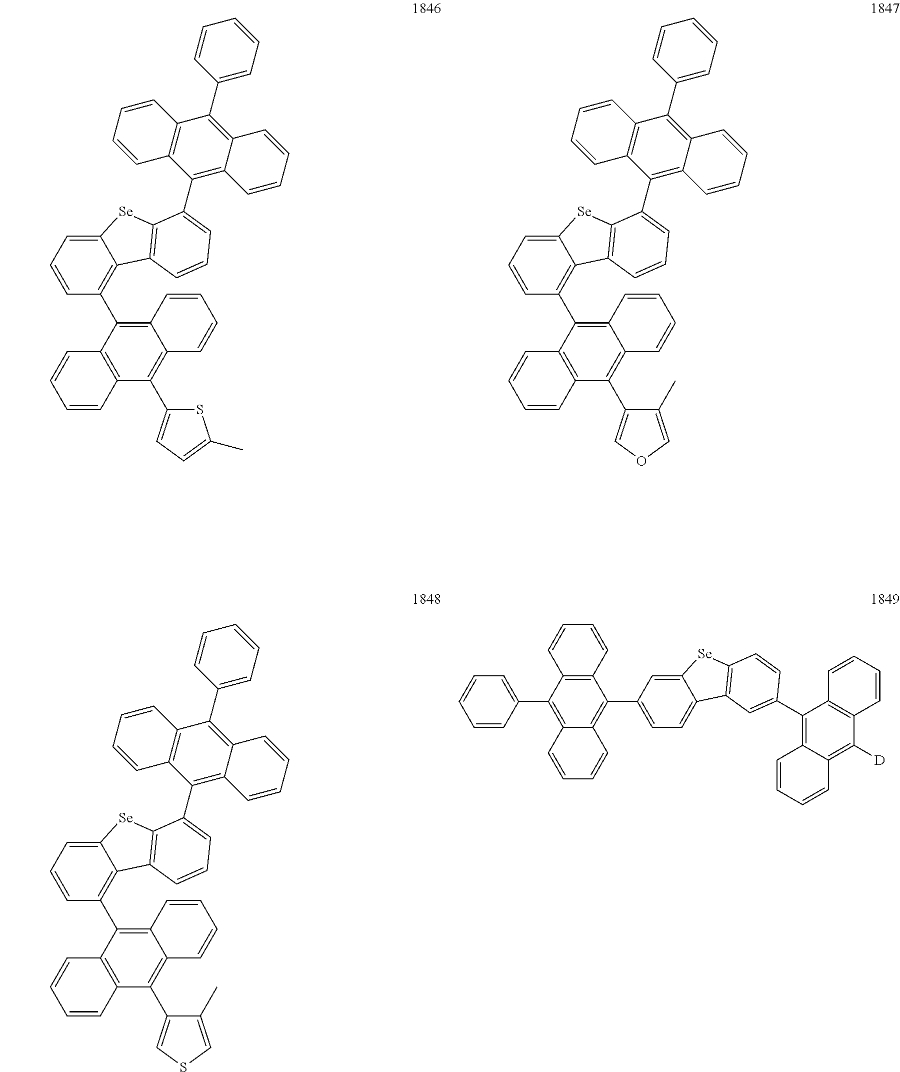

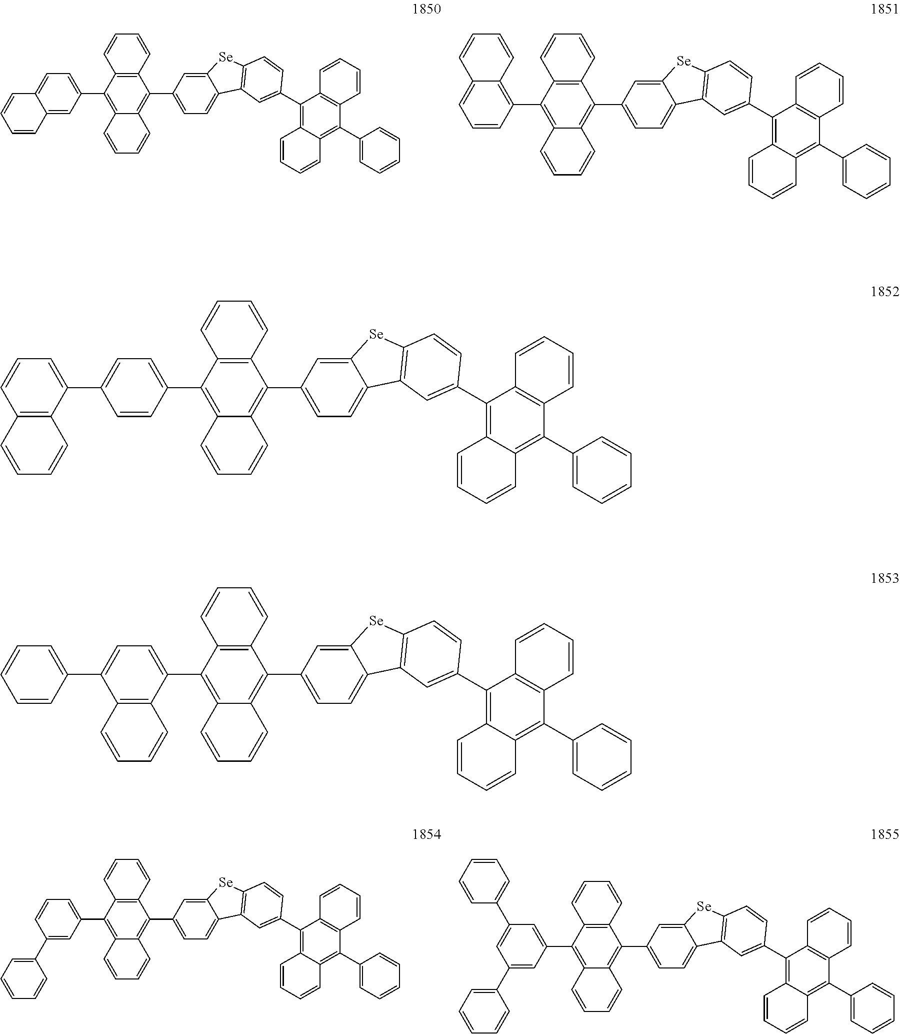

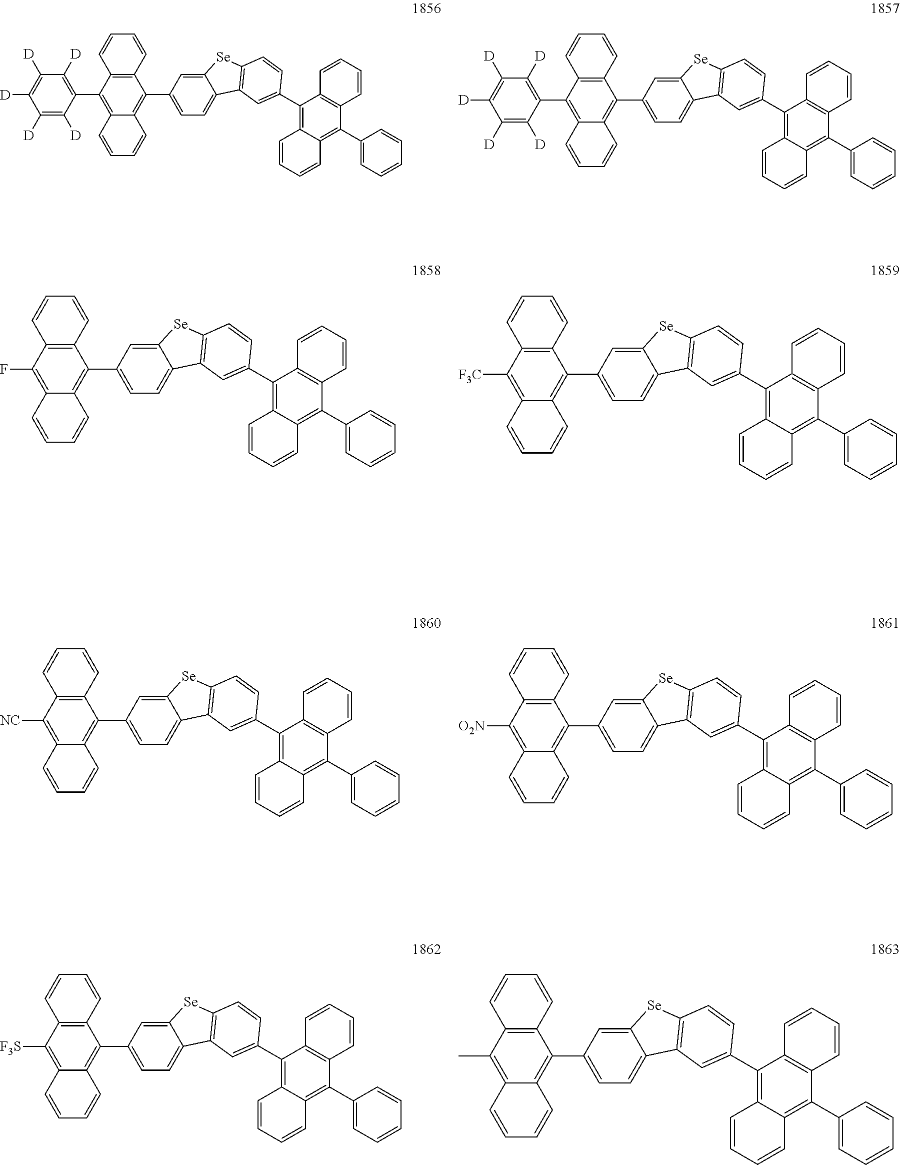

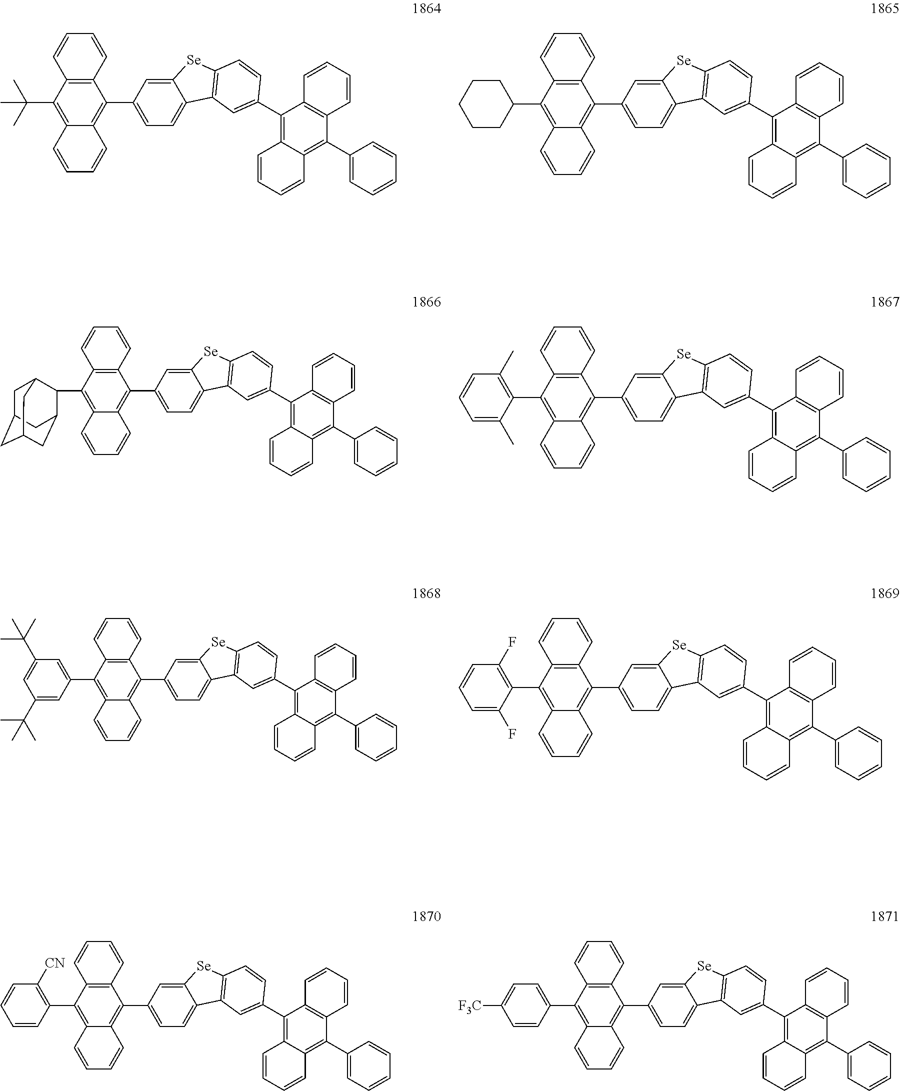

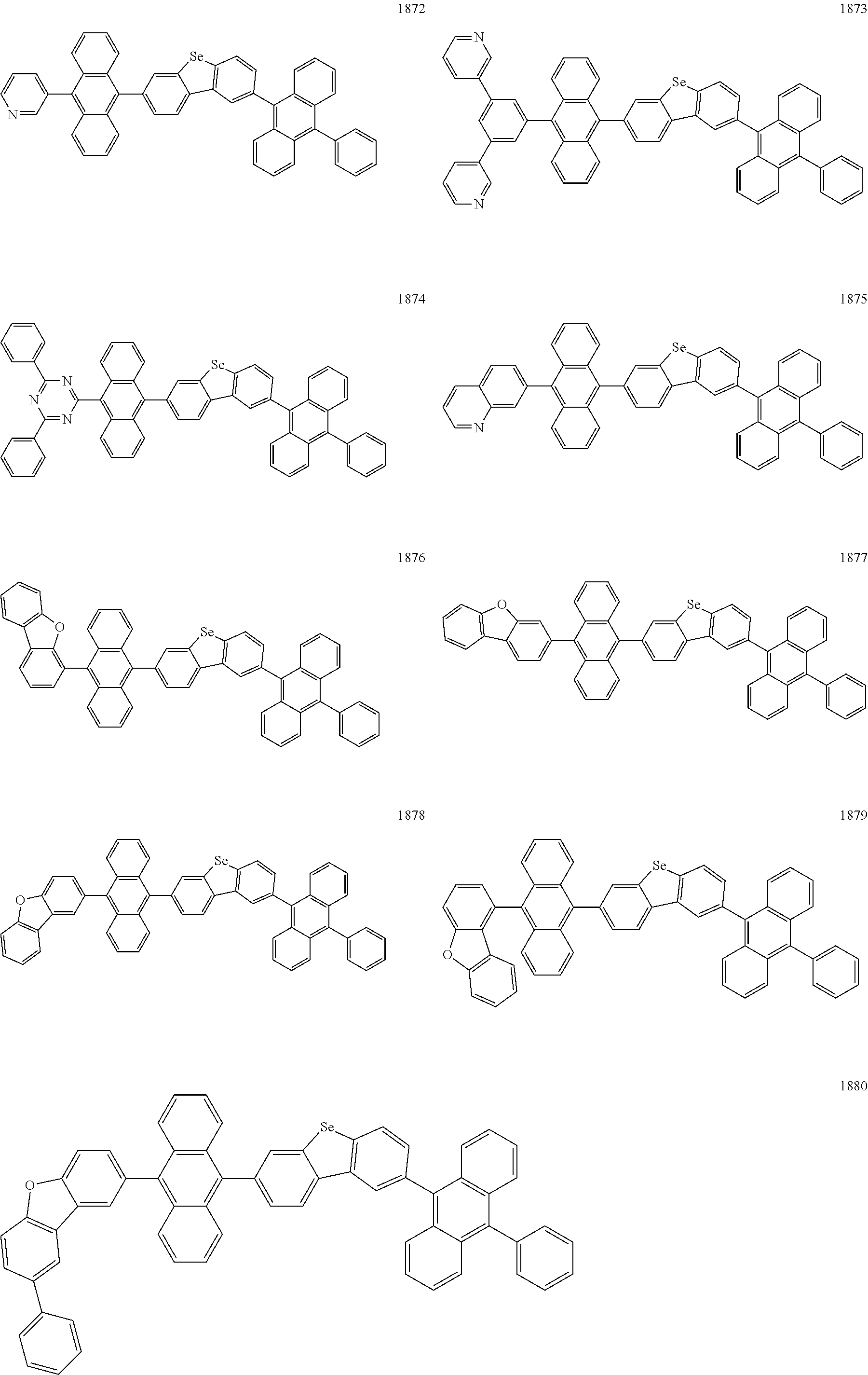

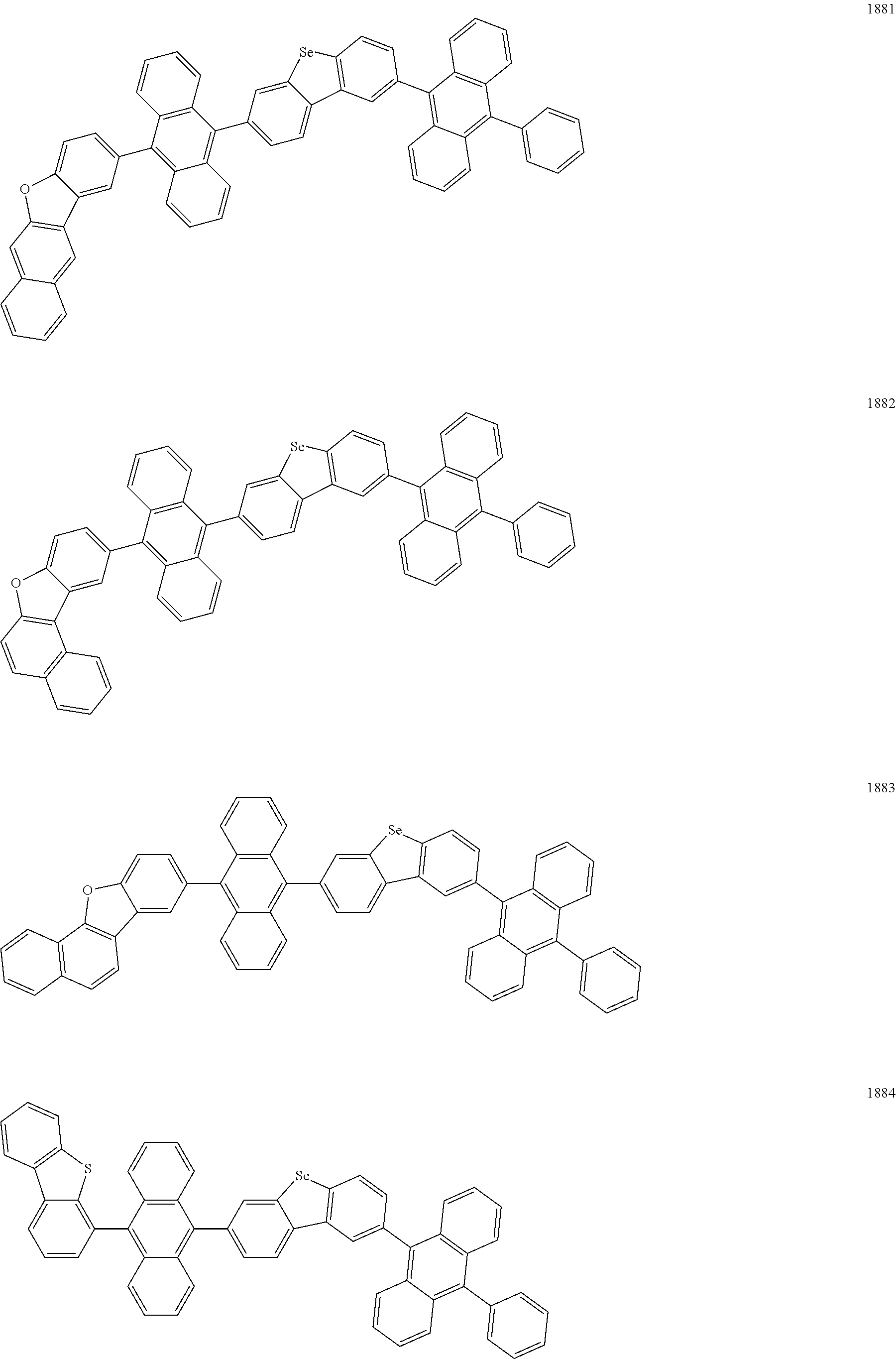 [SMILES: c1ccc(-c2c3ccccc3c(-c3ccc4[se]c5cc(-c6c7ccccc7c(-c7ccc8oc9cc%10ccccc%10cc9c8c7)c7ccccc67)ccc5c4c3)c3ccccc23)cc1 ; c1ccc(-c2c3ccccc3c(-c3ccc4[se]c5cc(-c6c7ccccc7c(-c7ccc8oc9ccc%10ccccc%10c9c8c7)c7ccccc67)ccc5c4c3)c3ccccc23)cc1 ; c1ccc(-c2c3ccccc3c(-c3ccc4[se]c5cc(-c6c7ccccc7c(-c7ccc8oc9c%10ccccc%10ccc9c8c7)c7ccccc67)ccc5c4c3)c3ccccc23)cc1 ; c1ccc(-c2c3ccccc3c(-c3ccc4[se]c5cc(-c6c7ccccc7c(-c7cccc8c7sc7ccccc78)c7ccccc67)ccc5c4c3)c3ccccc23)cc1]

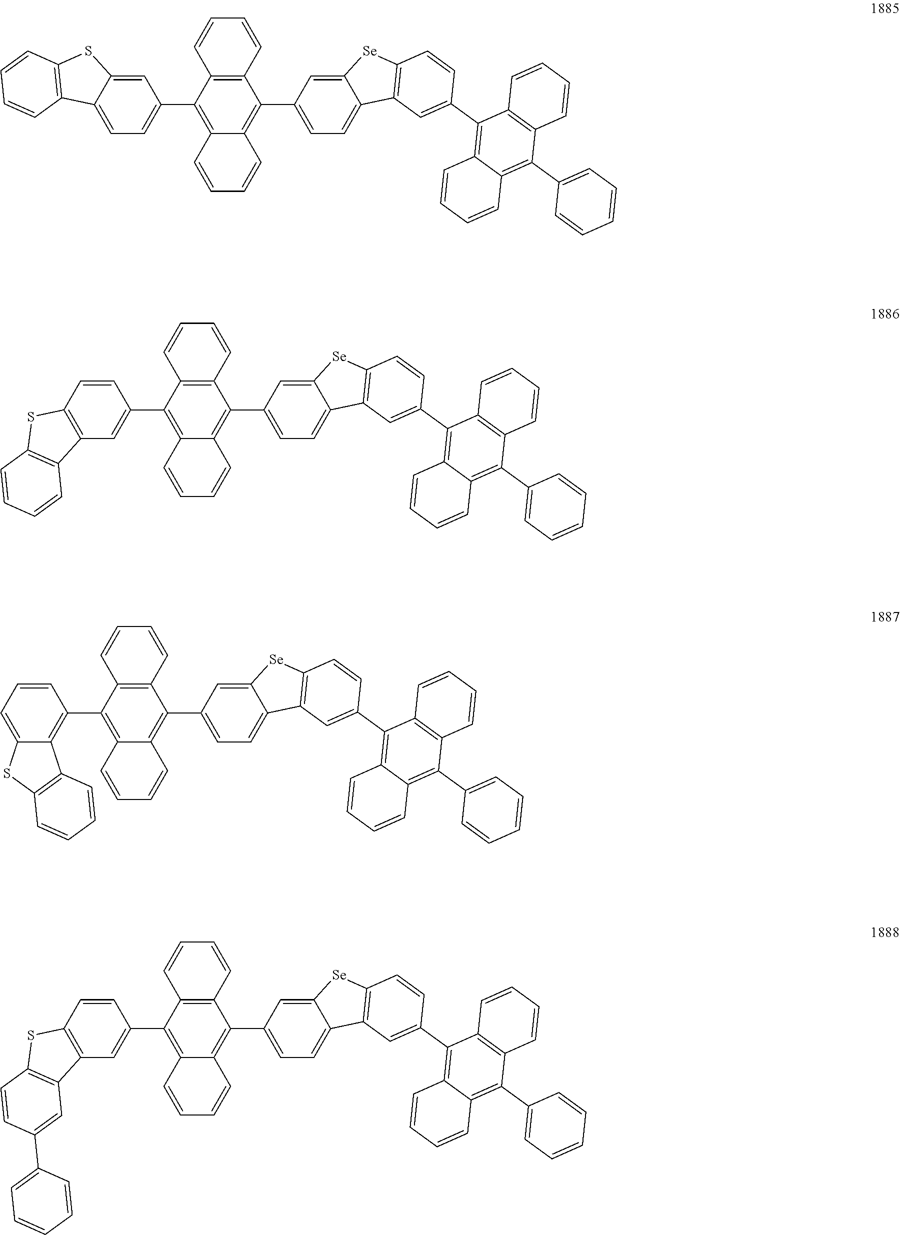 [SMILES: c1ccc(-c2c3ccccc3c(-c3ccc4[se]c5cc(-c6c7ccccc7c(-c7ccc8c(c7)sc7ccccc78)c7ccccc67)ccc5c4c3)c3ccccc23)cc1 ; c1ccc(-c2c3ccccc3c(-c3ccc4[se]c5cc(-c6c7ccccc7c(-c7ccc8sc9ccccc9c8c7)c7ccccc67)ccc5c4c3)c3ccccc23)cc1 ; c1ccc(-c2c3ccccc3c(-c3ccc4[se]c5cc(-c6c7ccccc7c(-c7cccc8sc9ccccc9c78)c7ccccc67)ccc5c4c3)c3ccccc23)cc1 ; c1ccc(-c2ccc3sc4ccc(-c5c6ccccc6c(-c6ccc7c(c6)[se]c6ccc(-c8c9ccccc9c(-c9ccccc9)c9ccccc89)cc67)c6ccccc56)cc4c3c2)cc1]

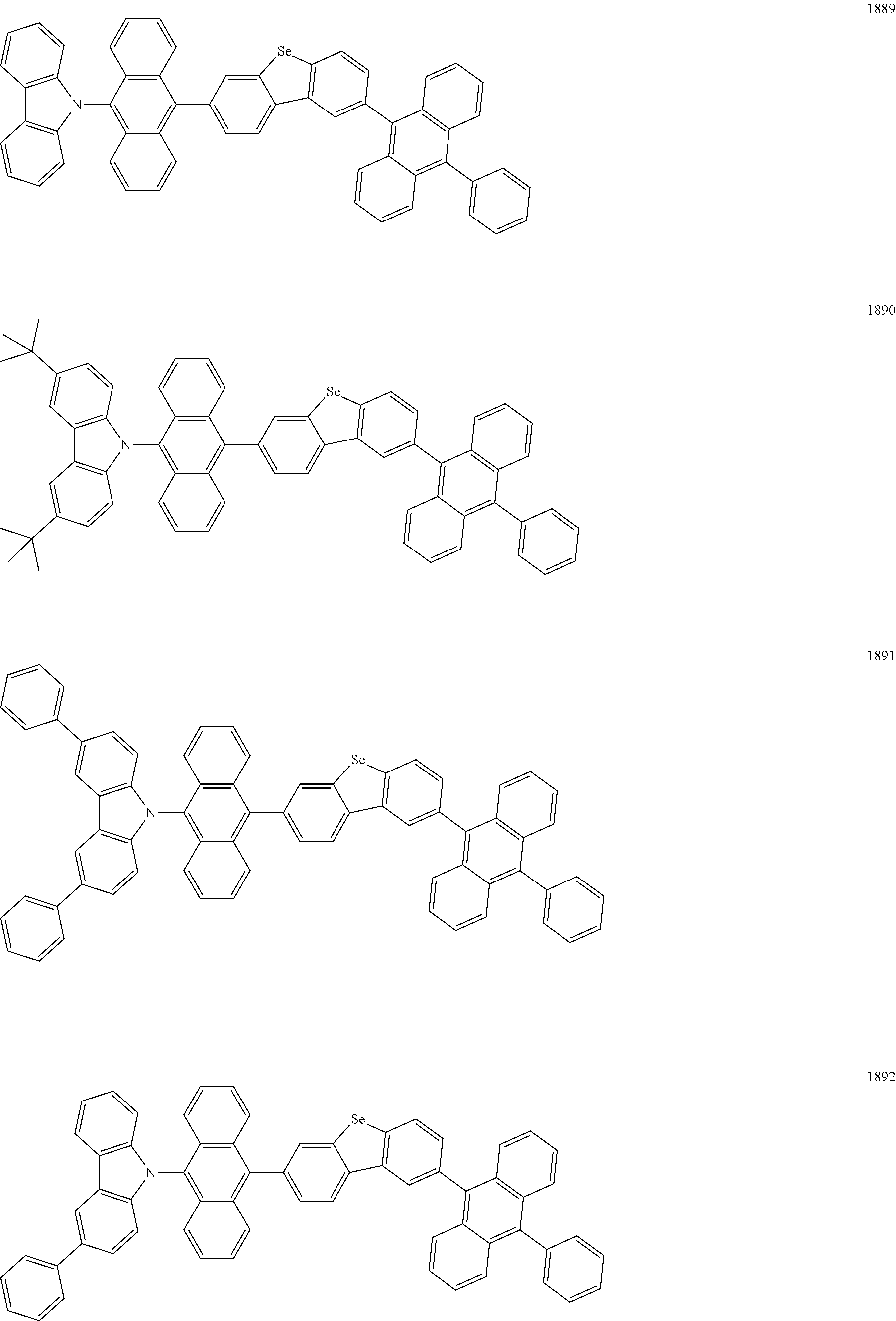 [SMILES: c1ccc(-c2c3ccccc3c(-c3ccc4[se]c5cc(-c6c7ccccc7c(-n7c8ccccc8c8ccccc87)c7ccccc67)ccc5c4c3)c3ccccc23)cc1 ; CC(C)(C)c1ccc2c(c1)c1cc(C(C)(C)C)ccc1n2-c1c2ccccc2c(-c2ccc3c(c2)[se]c2ccc(-c4c5ccccc5c(-c5ccccc5)c5ccccc45)cc23)c2ccccc12 ; c1ccc(-c2ccc3c(c2)c2cc(-c4ccccc4)ccc2n3-c2c3ccccc3c(-c3ccc4c(c3)[se]c3ccc(-c5c6ccccc6c(-c6ccccc6)c6ccccc56)cc34)c3ccccc23)cc1 ; c1ccc(-c2ccc3c(c2)c2ccccc2n3-c2c3ccccc3c(-c3ccc4c(c3)[se]c3ccc(-c5c6ccccc6c(-c6ccccc6)c6ccccc56)cc34)c3ccccc23)cc1]

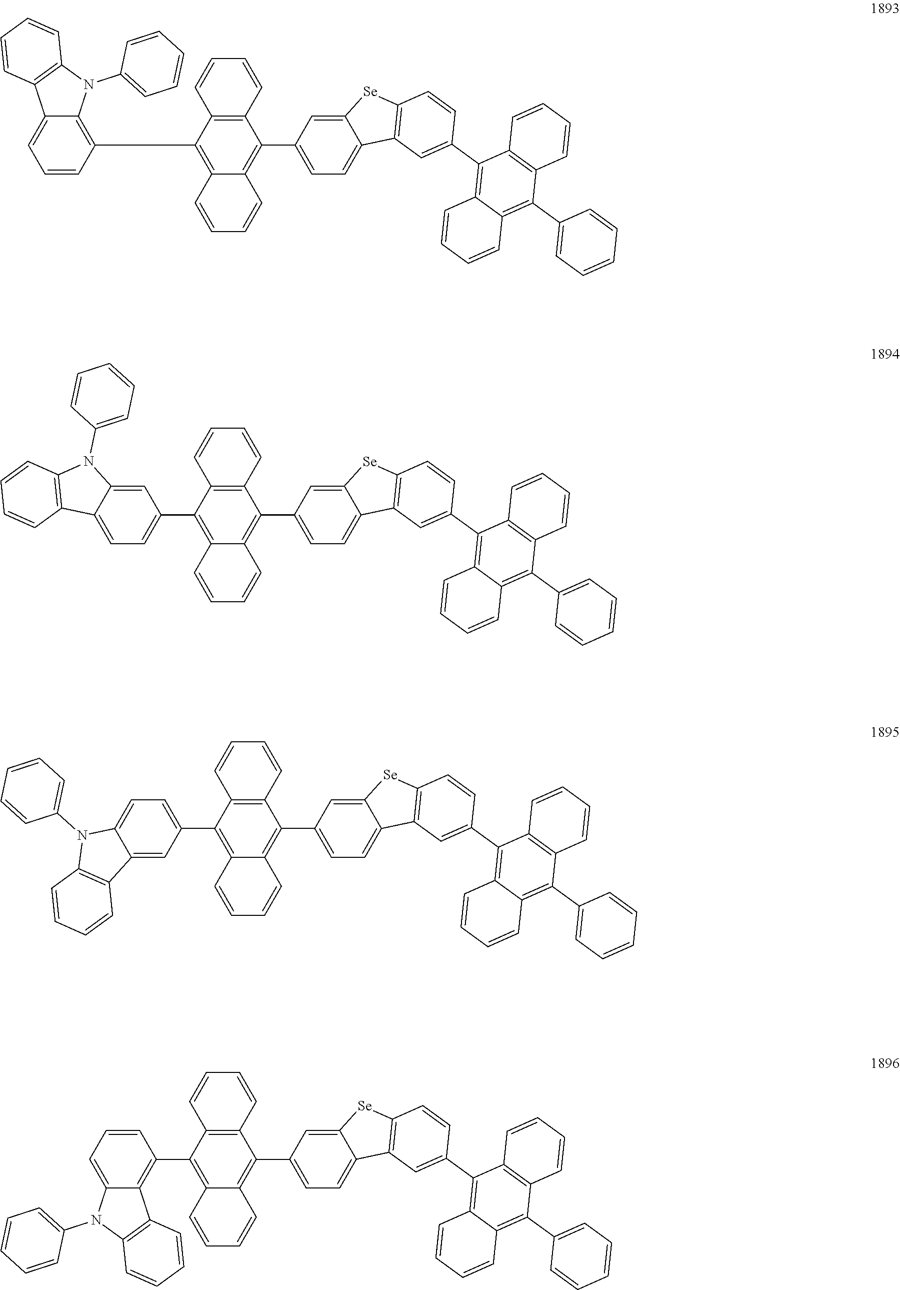 [SMILES: c1ccc(-c2c3ccccc3c(-c3ccc4[se]c5cc(-c6c7ccccc7c(-c7cccc8c9ccccc9n(-c9ccccc9)c78)c7ccccc67)ccc5c4c3)c3ccccc23)cc1 ; c1ccc(-c2c3ccccc3c(-c3ccc4[se]c5cc(-c6c7ccccc7c(-c7ccc8c9ccccc9n(-c9ccccc9)c8c7)c7ccccc67)ccc5c4c3)c3ccccc23)cc1 ; c1ccc(-c2c3ccccc3c(-c3ccc4[se]c5cc(-c6c7ccccc7c(-c7ccc8c(c7)c7ccccc7n8-c7ccccc7)c7ccccc67)ccc5c4c3)c3ccccc23)cc1 ; c1ccc(-c2c3ccccc3c(-c3ccc4[se]c5cc(-c6c7ccccc7c(-c7cccc8c7c7ccccc7n8-c7ccccc7)c7ccccc67)ccc5c4c3)c3ccccc23)cc1]

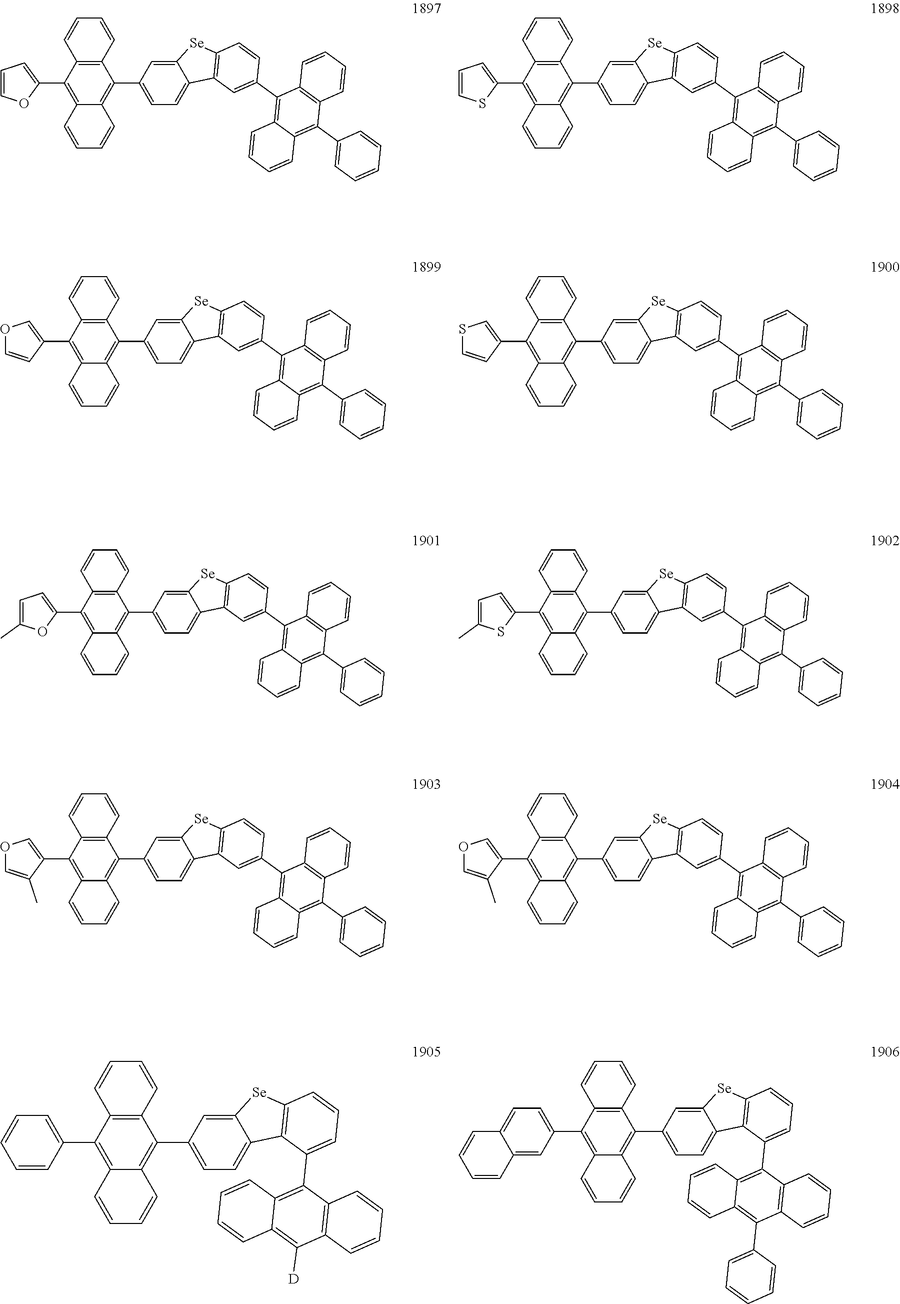

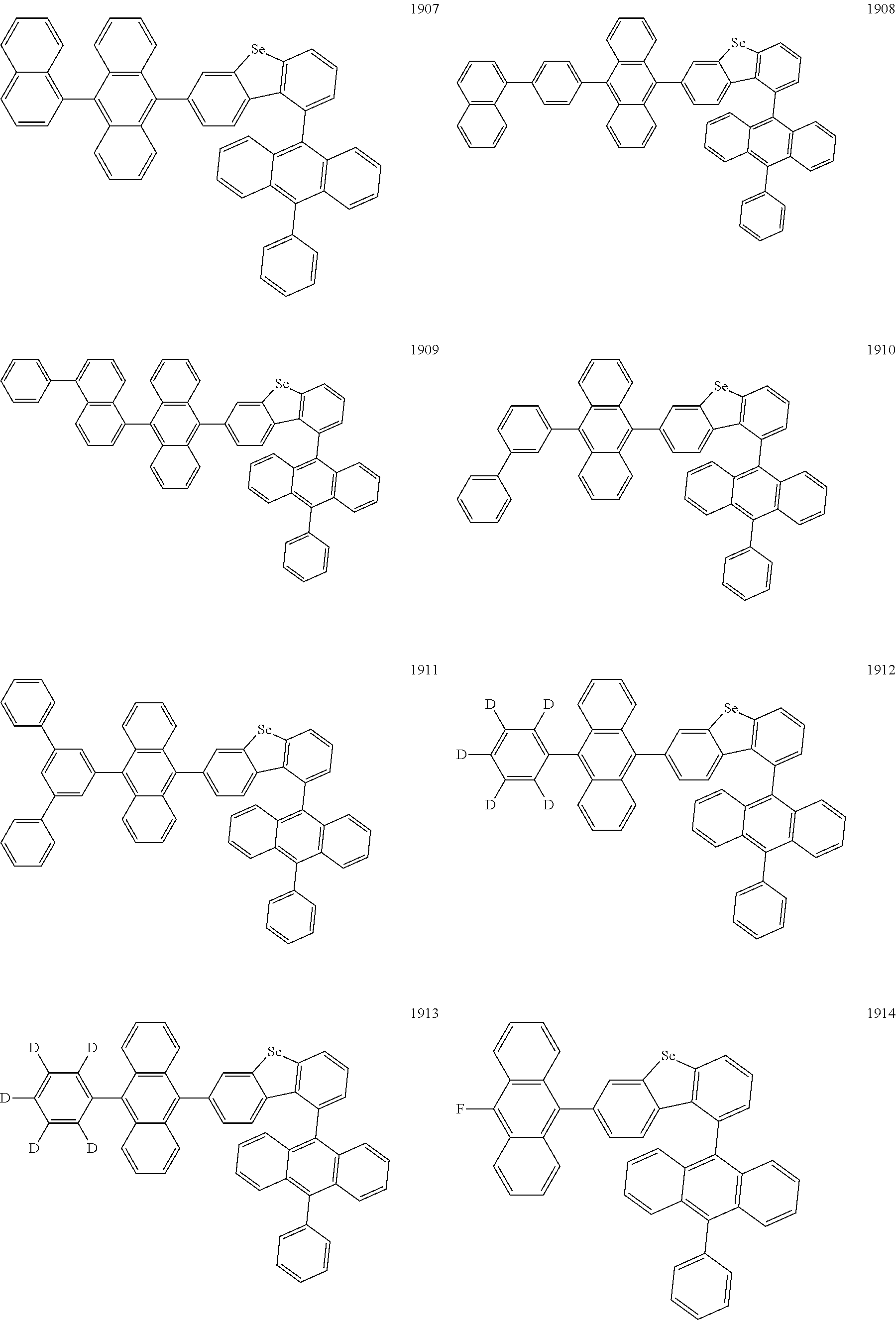

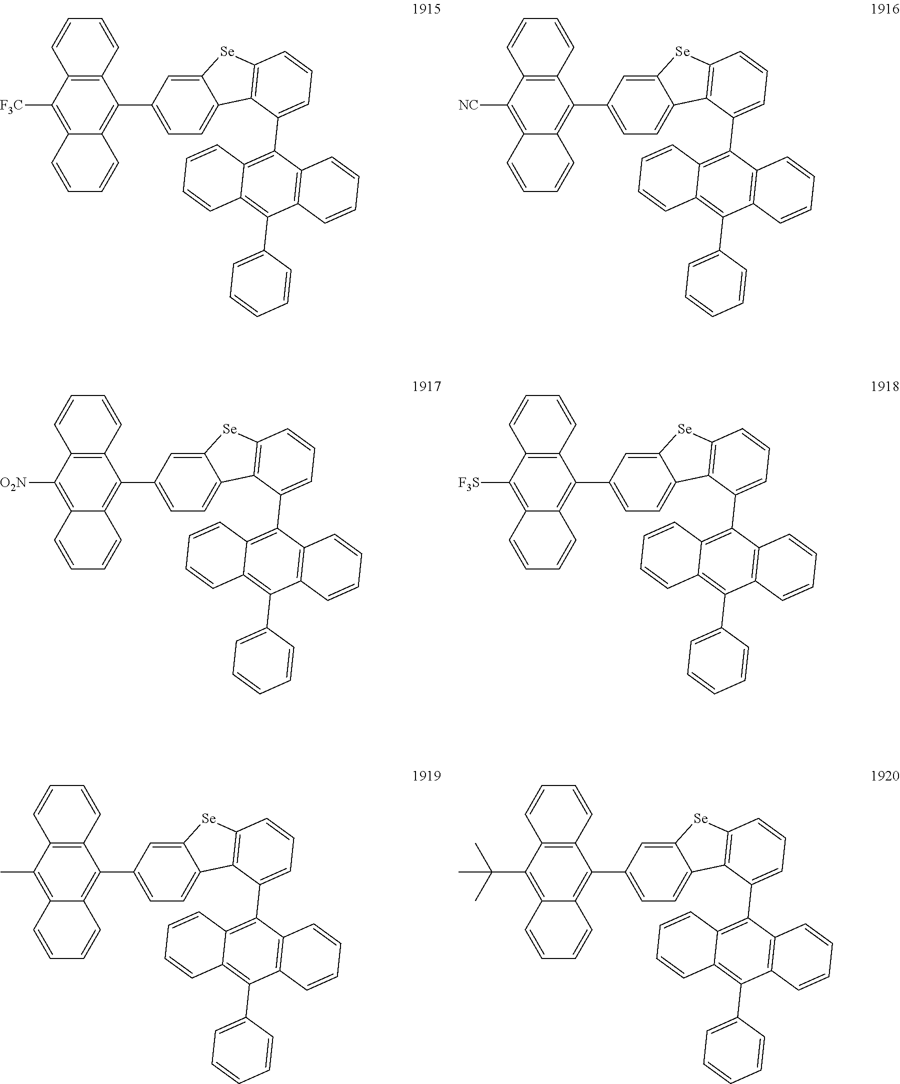

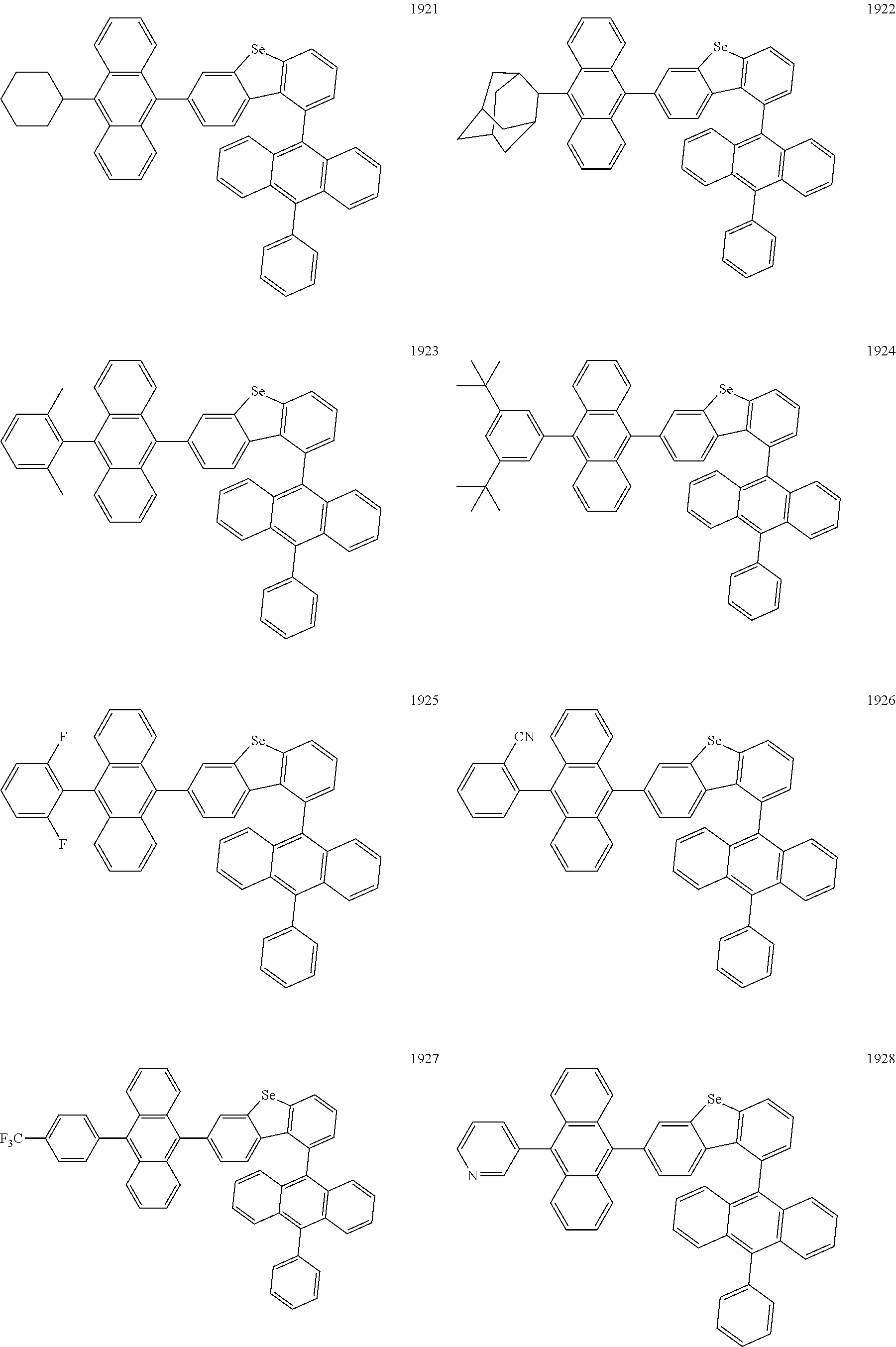

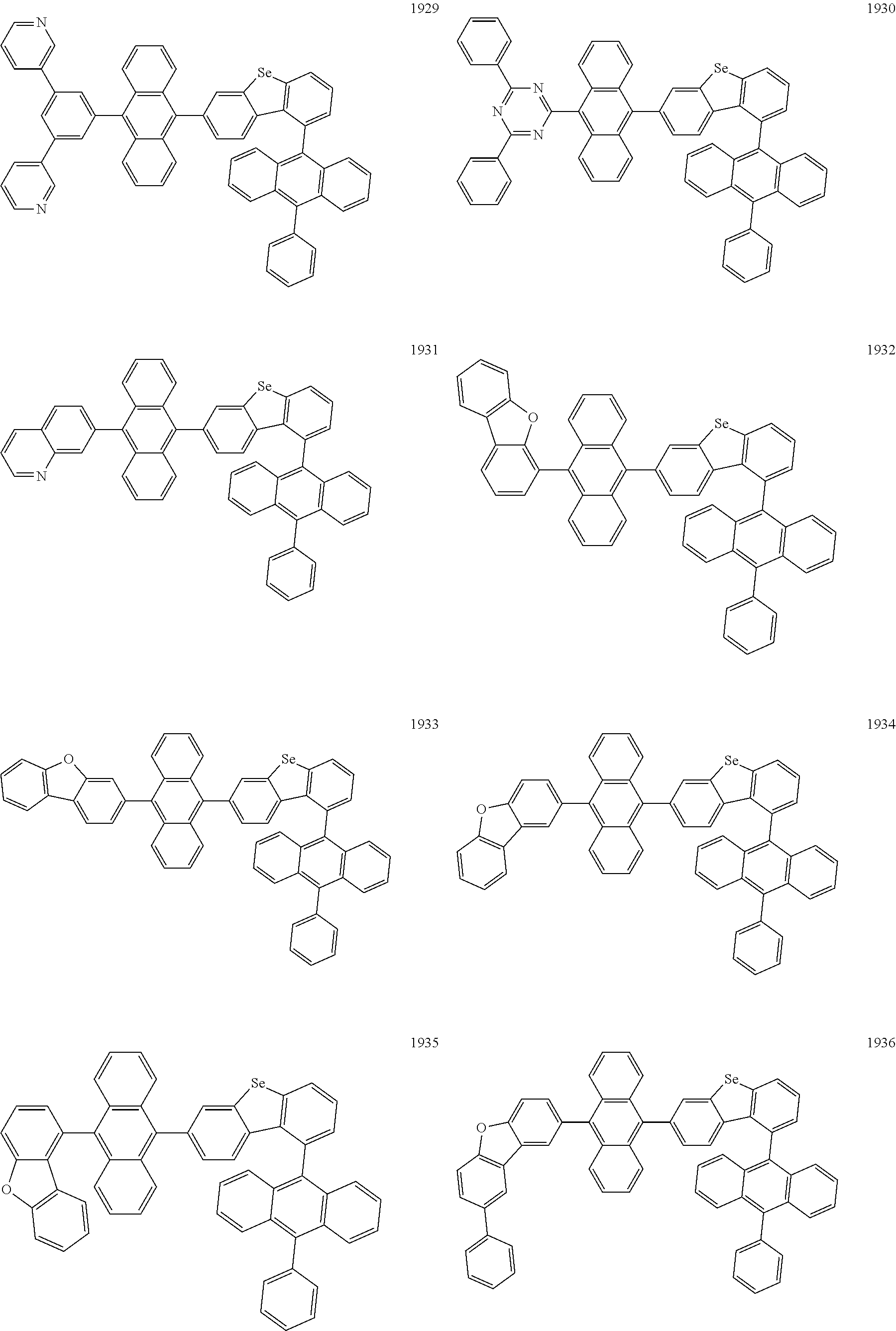

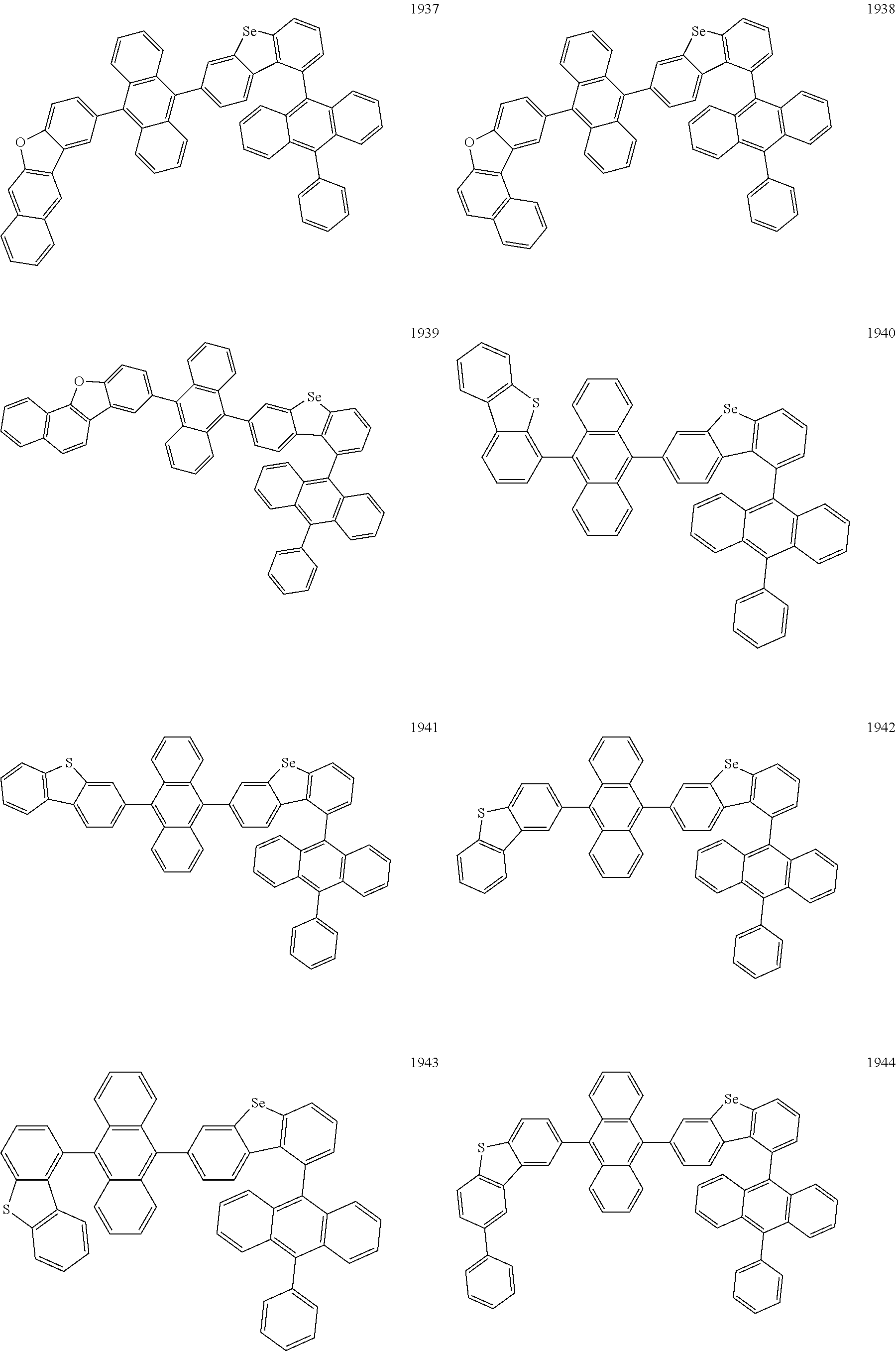

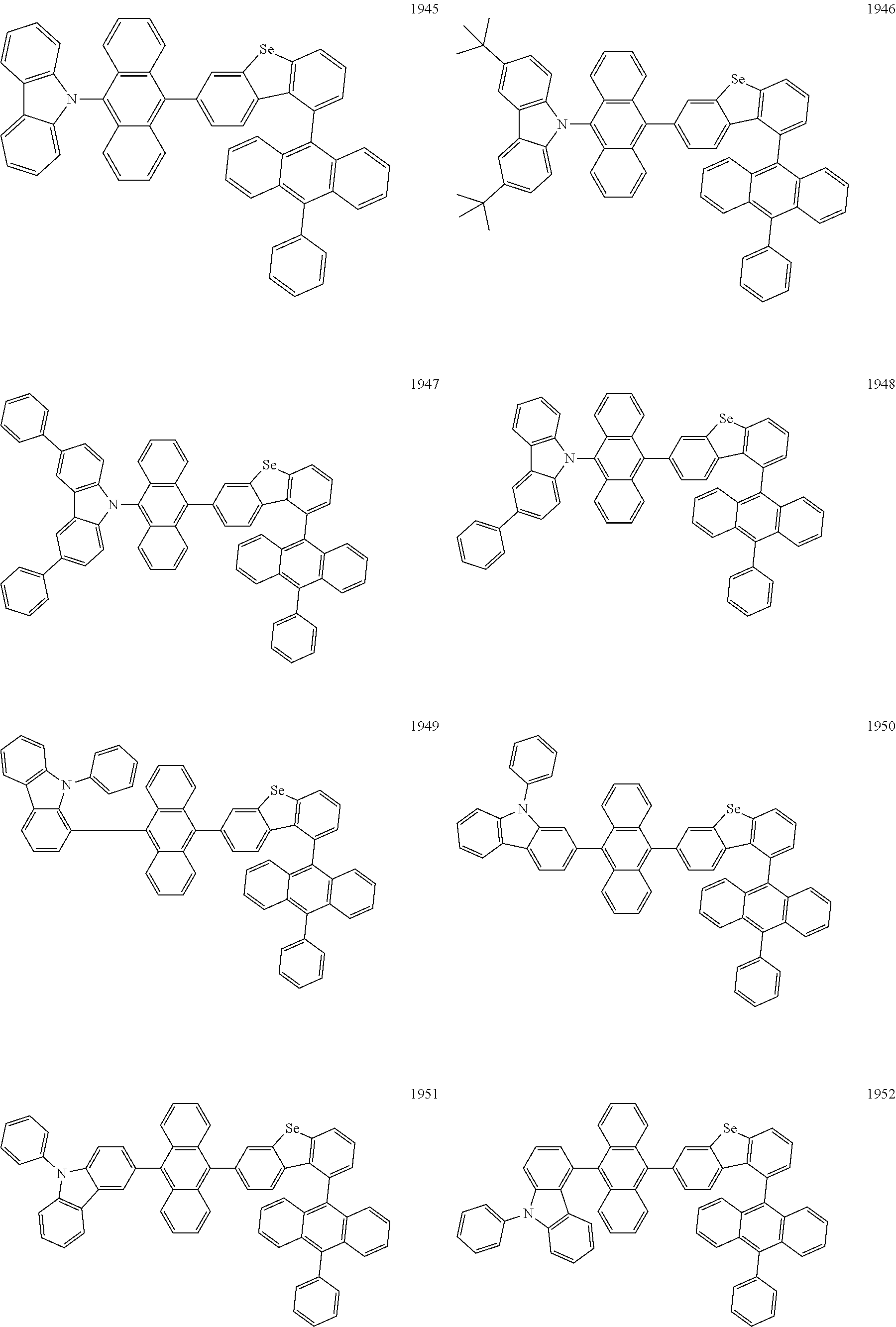

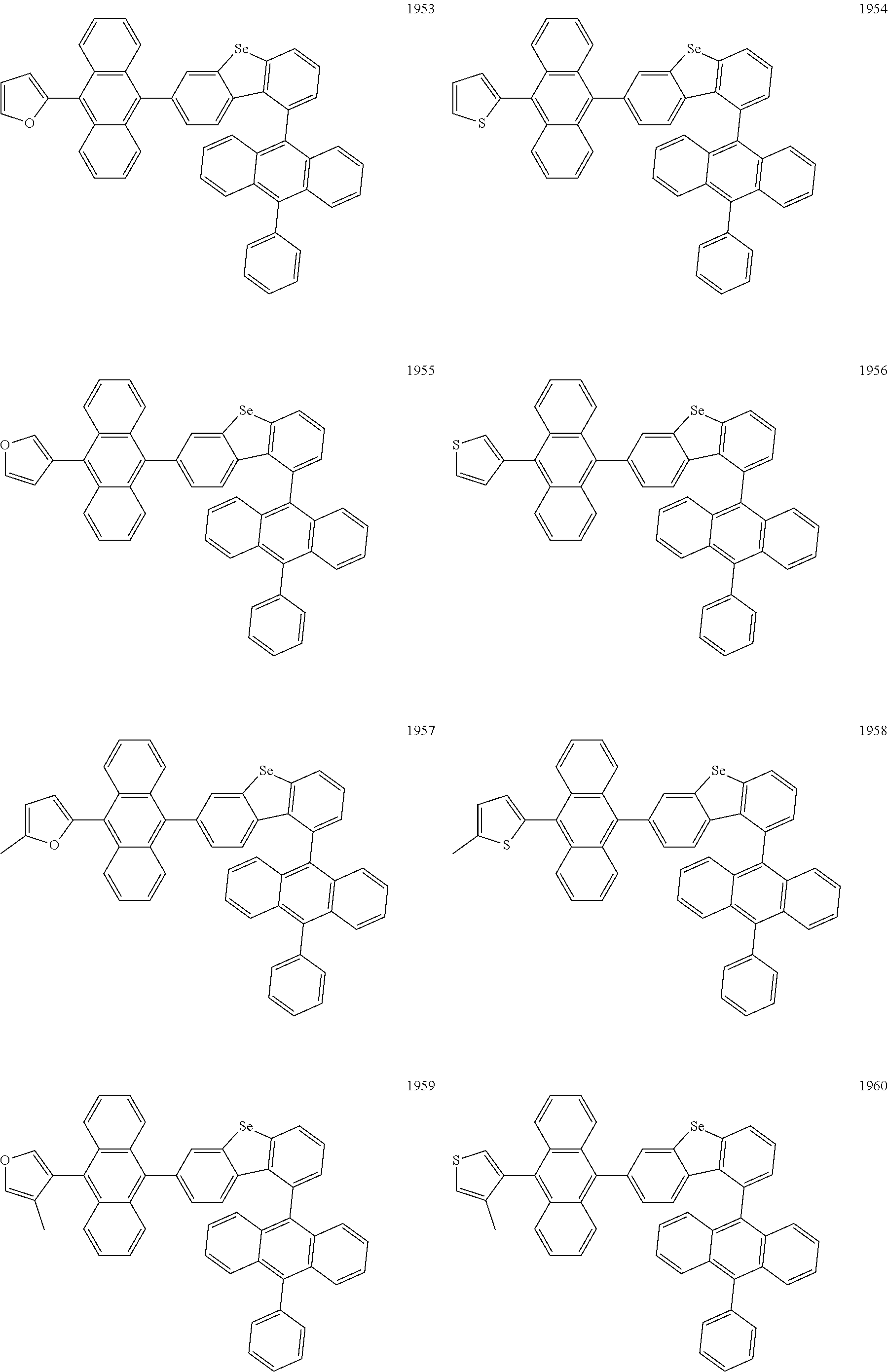

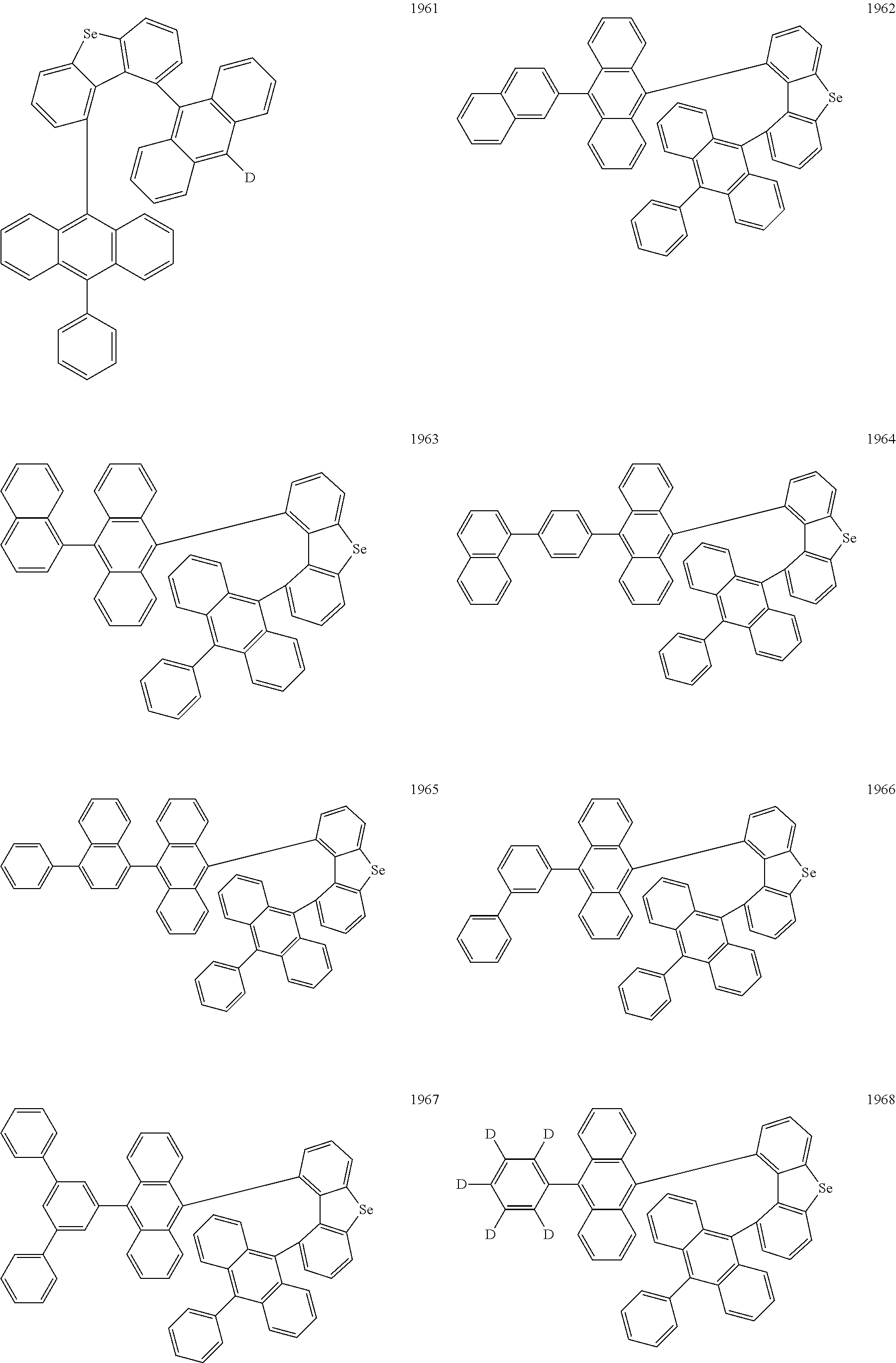

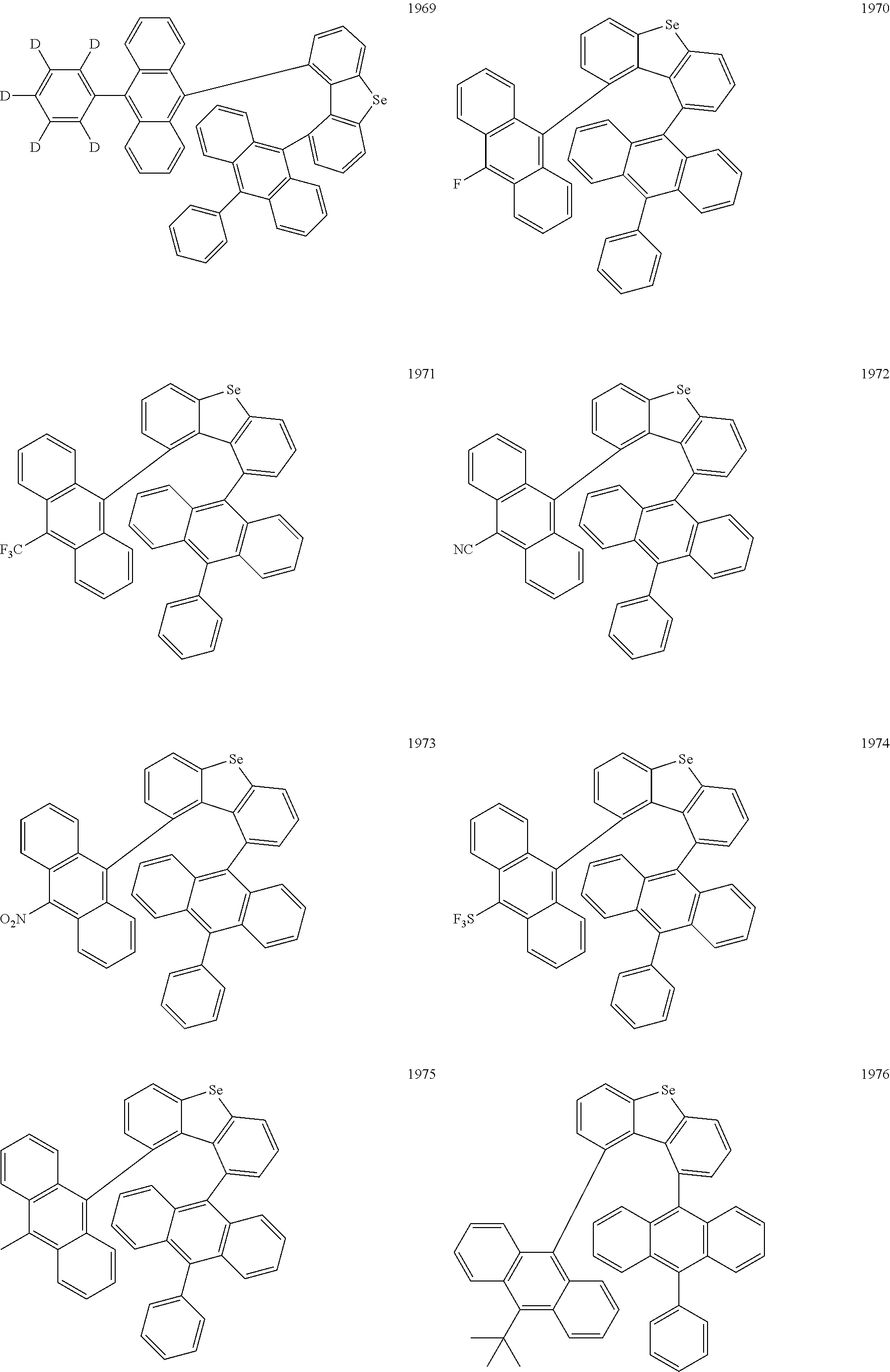

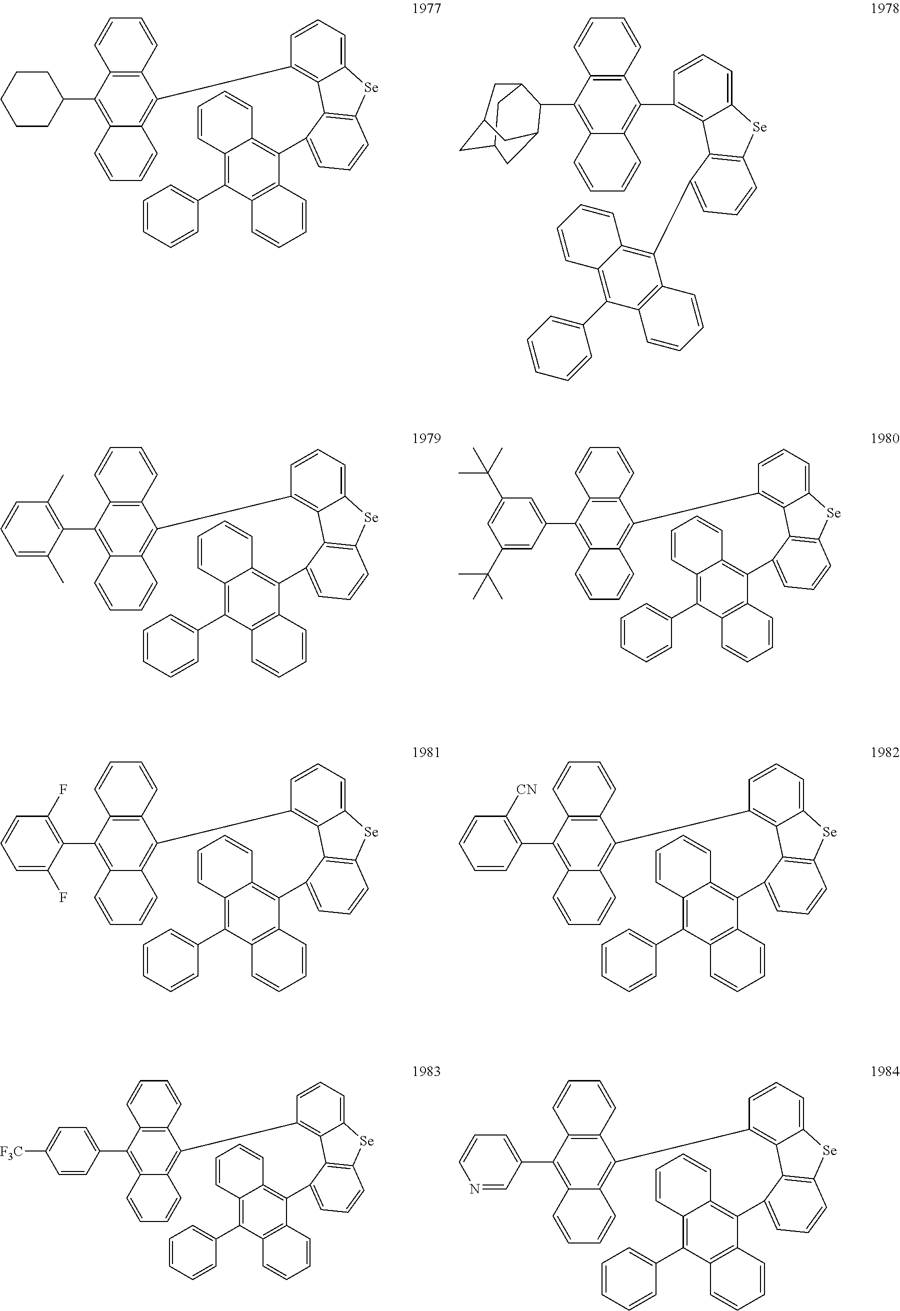

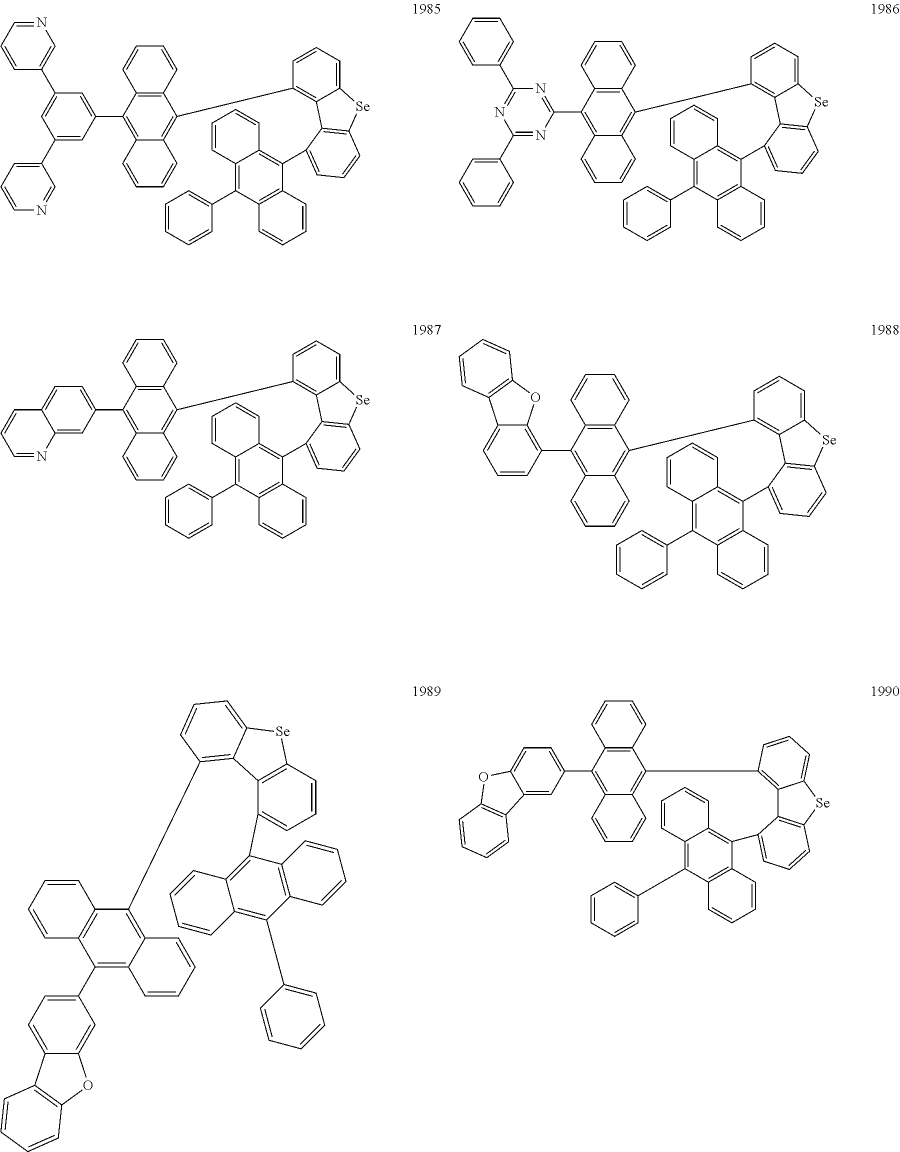

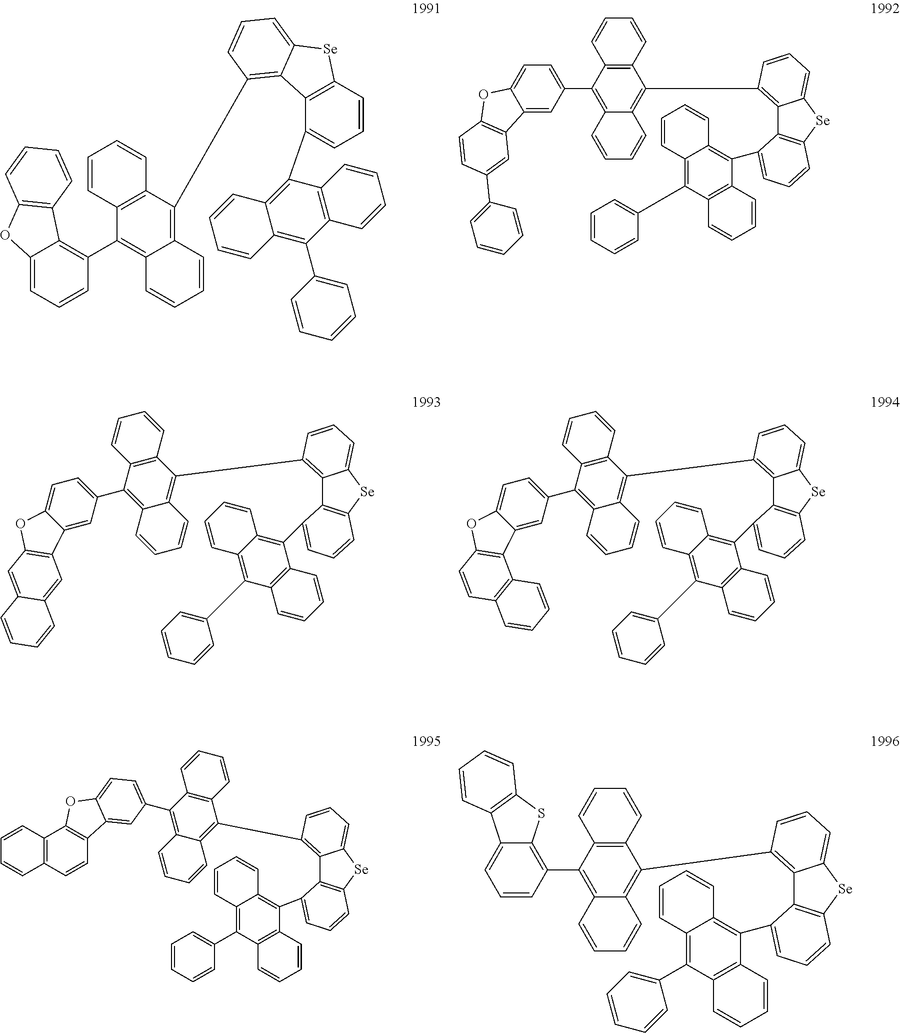

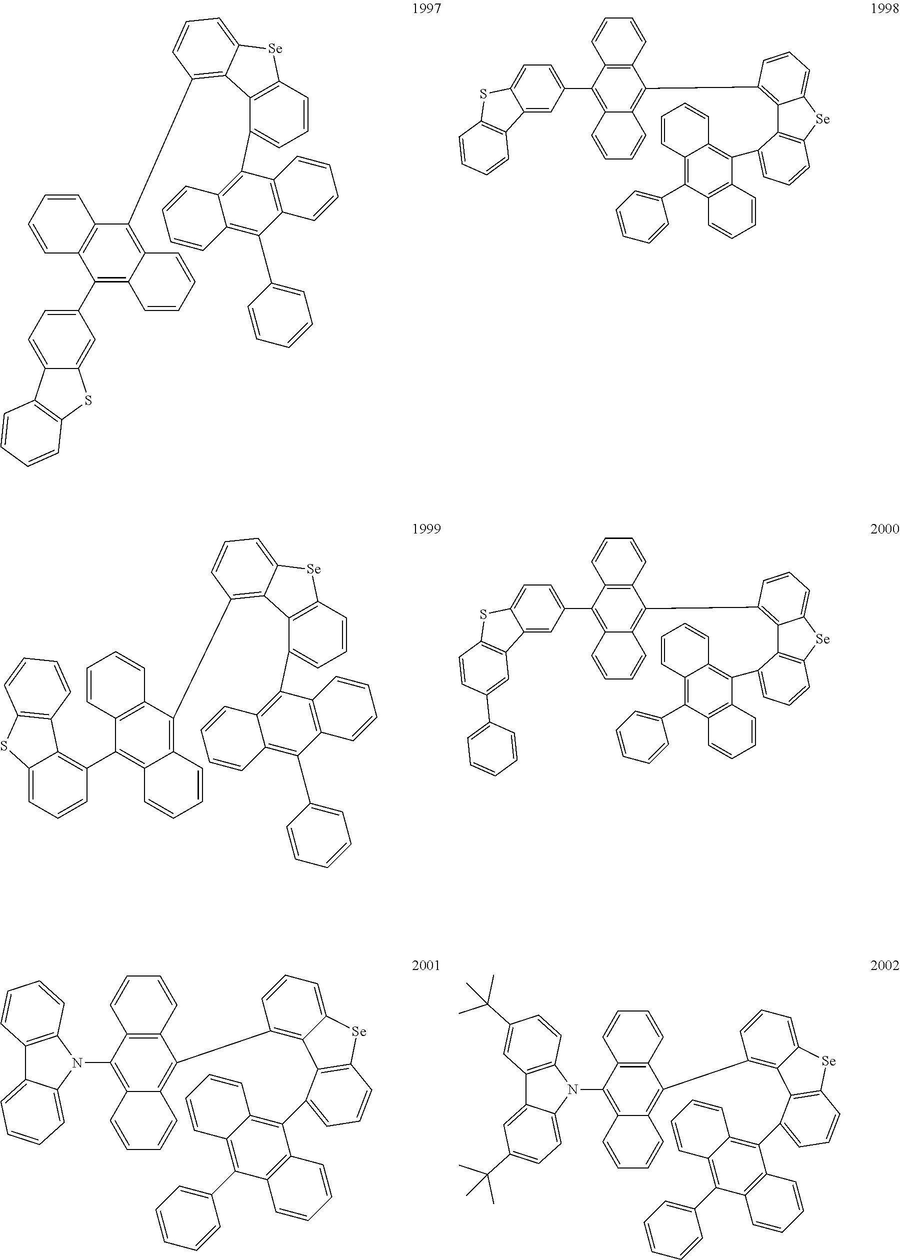

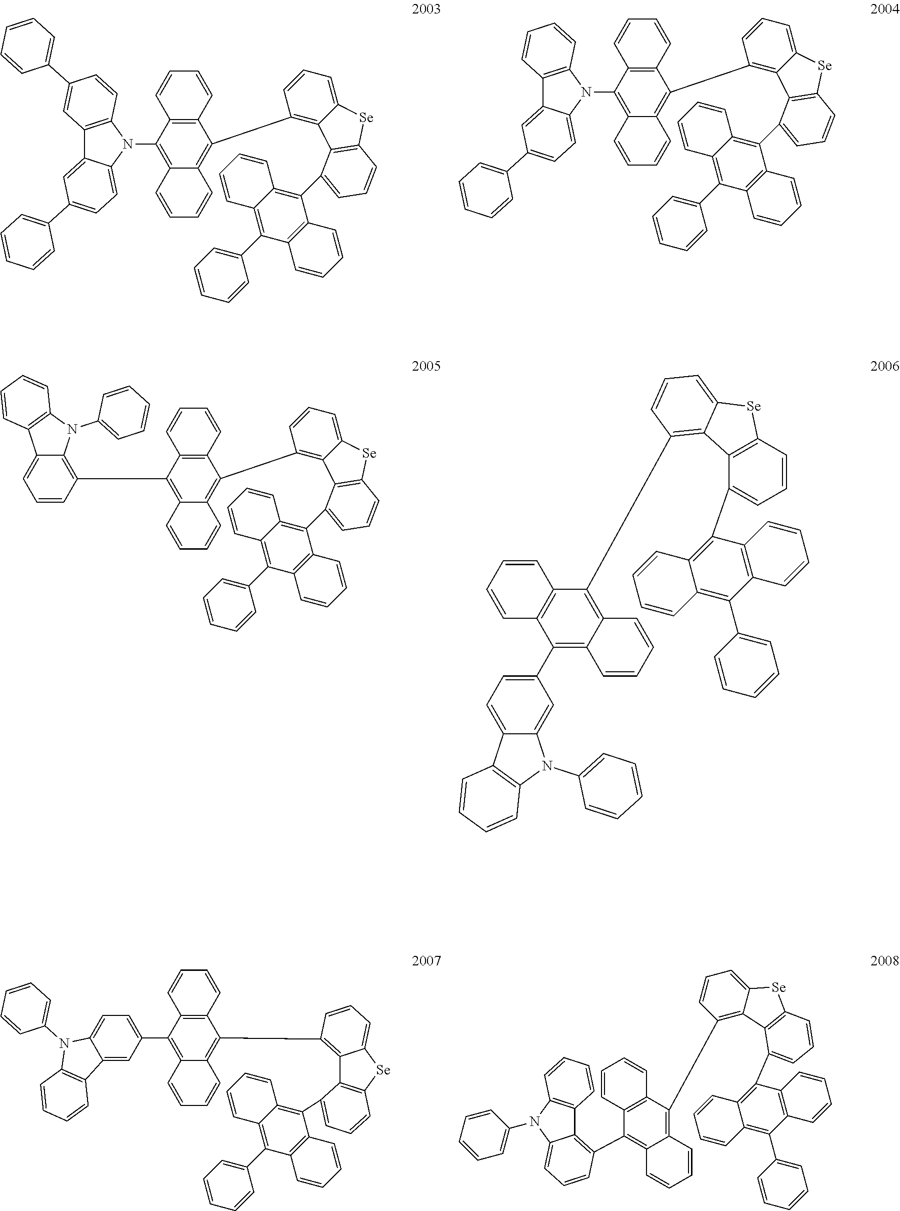

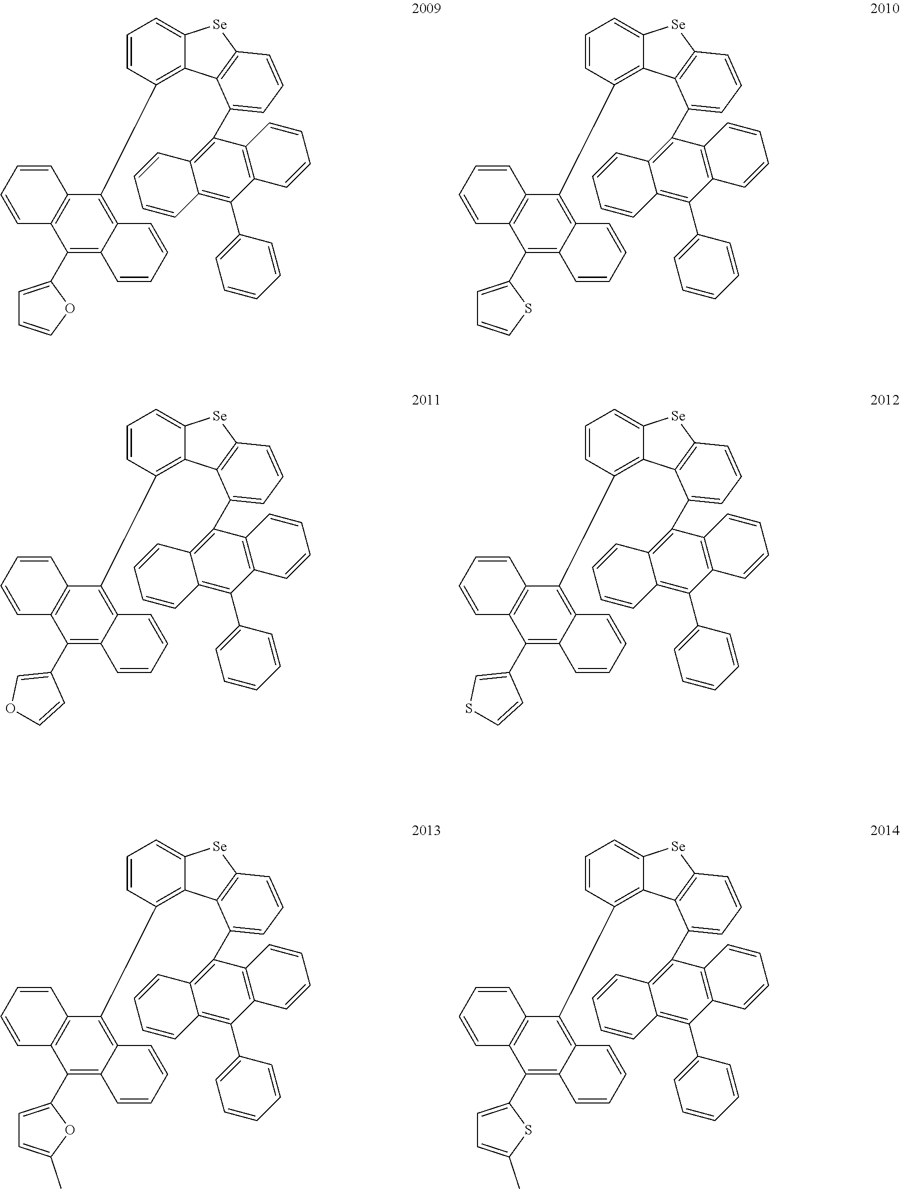

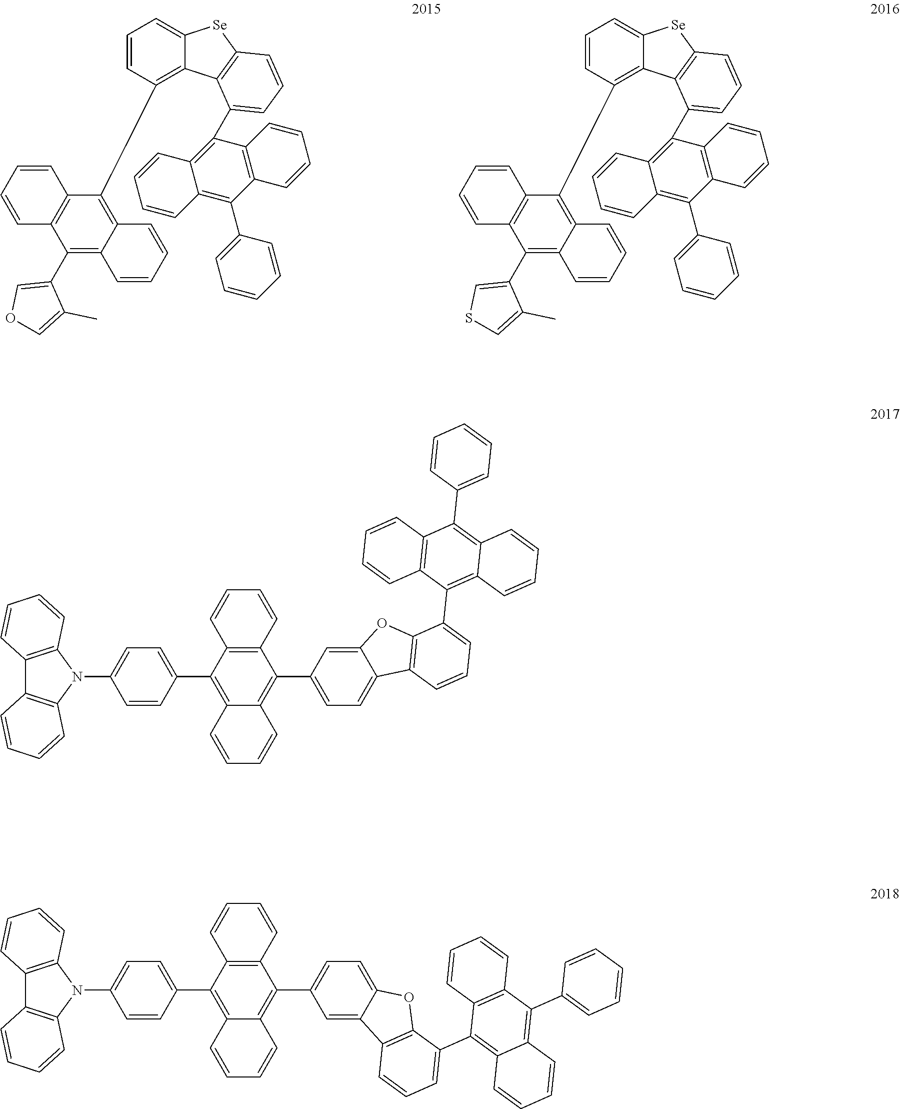 [SMILES: Cc1cocc1-c1c2ccccc2c(-c2cccc3[se]c4cccc(-c5c6ccccc6c(-c6ccccc6)c6ccccc56)c4c23)c2ccccc12 ; Cc1cscc1-c1c2ccccc2c(-c2cccc3[se]c4cccc(-c5c6ccccc6c(-c6ccccc6)c6ccccc56)c4c23)c2ccccc12 ; c1ccc(-c2c3ccccc3c(-c3cccc4c3oc3cc(-c5c6ccccc6c(-c6ccc(-n7c8ccccc8c8ccccc87)cc6)c6ccccc56)ccc34)c3ccccc23)cc1 ; c1ccc(-c2c3ccccc3c(-c3cccc4c3oc3ccc(-c5c6ccccc6c(-c6ccc(-n7c8ccccc8c8ccccc87)cc6)c6ccccc56)cc34)c3ccccc23)cc1]

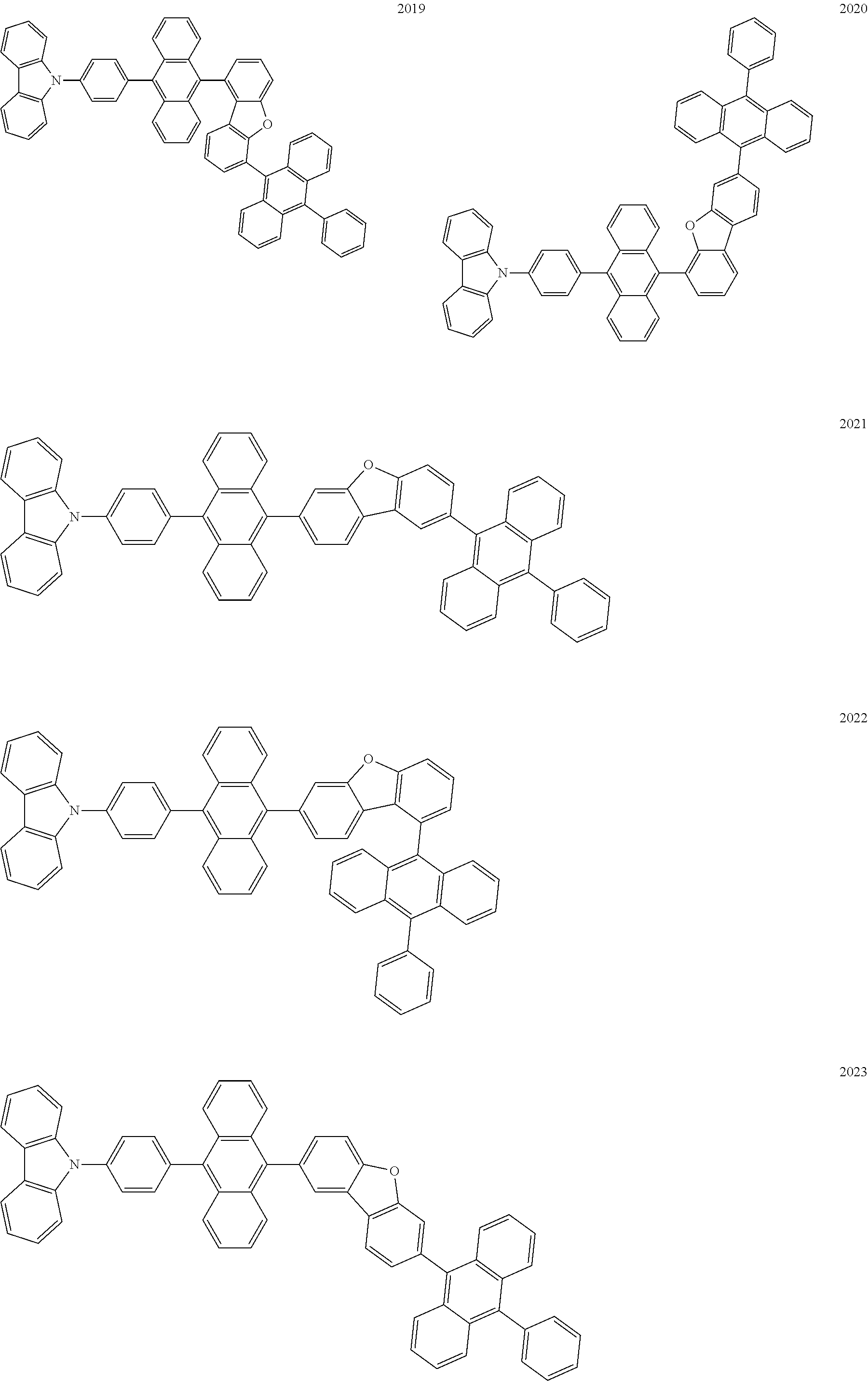

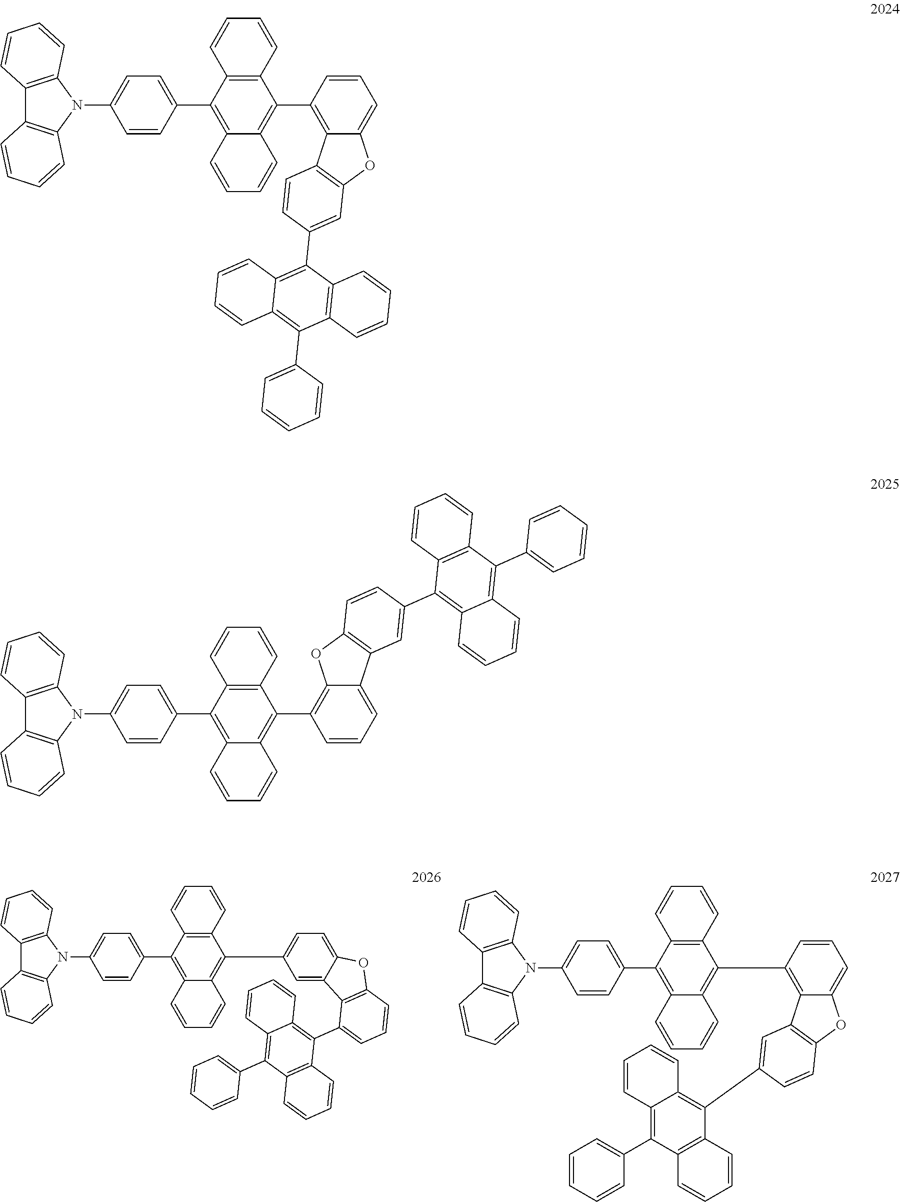 [SMILES: c1ccc(-c2c3ccccc3c(-c3ccc4c(c3)oc3cccc(-c5c6ccccc6c(-c6ccc(-n7c8ccccc8c8ccccc87)cc6)c6ccccc56)c34)c3ccccc23)cc1 ; c1ccc(-c2c3ccccc3c(-c3ccc4oc5c(-c6c7ccccc7c(-c7ccc(-n8c9ccccc9c9ccccc98)cc7)c7ccccc67)cccc5c4c3)c3ccccc23)cc1 ; c1ccc(-c2c3ccccc3c(-c3cccc4oc5ccc(-c6c7ccccc7c(-c7ccc(-n8c9ccccc9c9ccccc98)cc7)c7ccccc67)cc5c34)c3ccccc23)cc1 ; c1ccc(-c2c3ccccc3c(-c3ccc4oc5cccc(-c6c7ccccc7c(-c7ccc(-n8c9ccccc9c9ccccc98)cc7)c7ccccc67)c5c4c3)c3ccccc23)cc1]

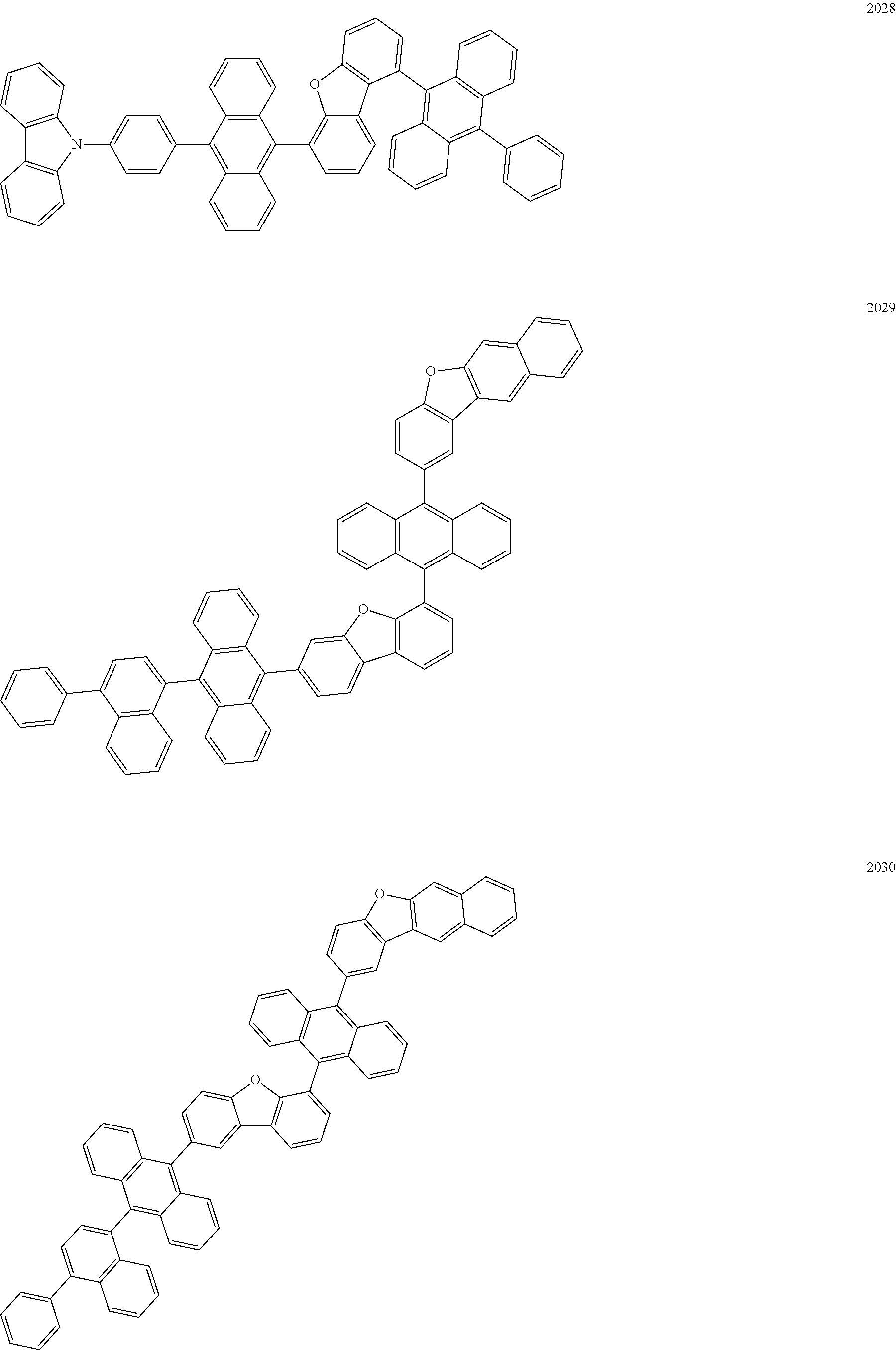 [SMILES: c1ccc(-c2c3ccccc3c(-c3cccc4oc5c(-c6c7ccccc7c(-c7ccc(-n8c9ccccc9c9ccccc98)cc7)c7ccccc67)cccc5c34)c3ccccc23)cc1 ; c1ccc(-c2ccc(-c3c4ccccc4c(-c4ccc5c(c4)oc4c(-c6c7ccccc7c(-c7ccc8oc9cc%10ccccc%10cc9c8c7)c7ccccc67)cccc45)c4ccccc34)c3ccccc23)cc1 ; c1ccc(-c2ccc(-c3c4ccccc4c(-c4ccc5oc6c(-c7c8ccccc8c(-c8ccc9oc%10cc%11ccccc%11cc%10c9c8)c8ccccc78)cccc6c5c4)c4ccccc34)c3ccccc23)cc1]

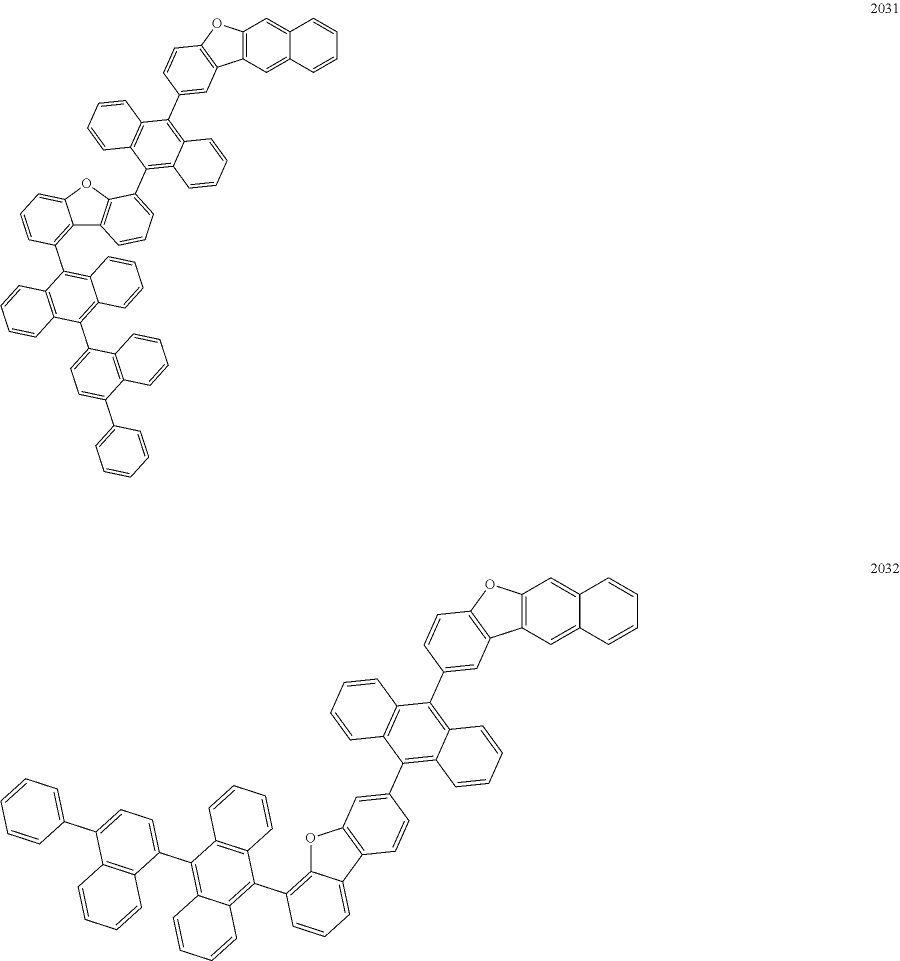 [SMILES: c1ccc(-c2ccc(-c3c4ccccc4c(-c4cccc5oc6c(-c7c8ccccc8c(-c8ccc9oc%10cc%11ccccc%11cc%10c9c8)c8ccccc78)cccc6c45)c4ccccc34)c3ccccc23)cc1 ; c1ccc(-c2ccc(-c3c4ccccc4c(-c4cccc5c4oc4cc(-c6c7ccccc7c(-c7ccc8oc9cc%10ccccc%10cc9c8c7)c7ccccc67)ccc45)c4ccccc34)c3ccccc23)cc1]

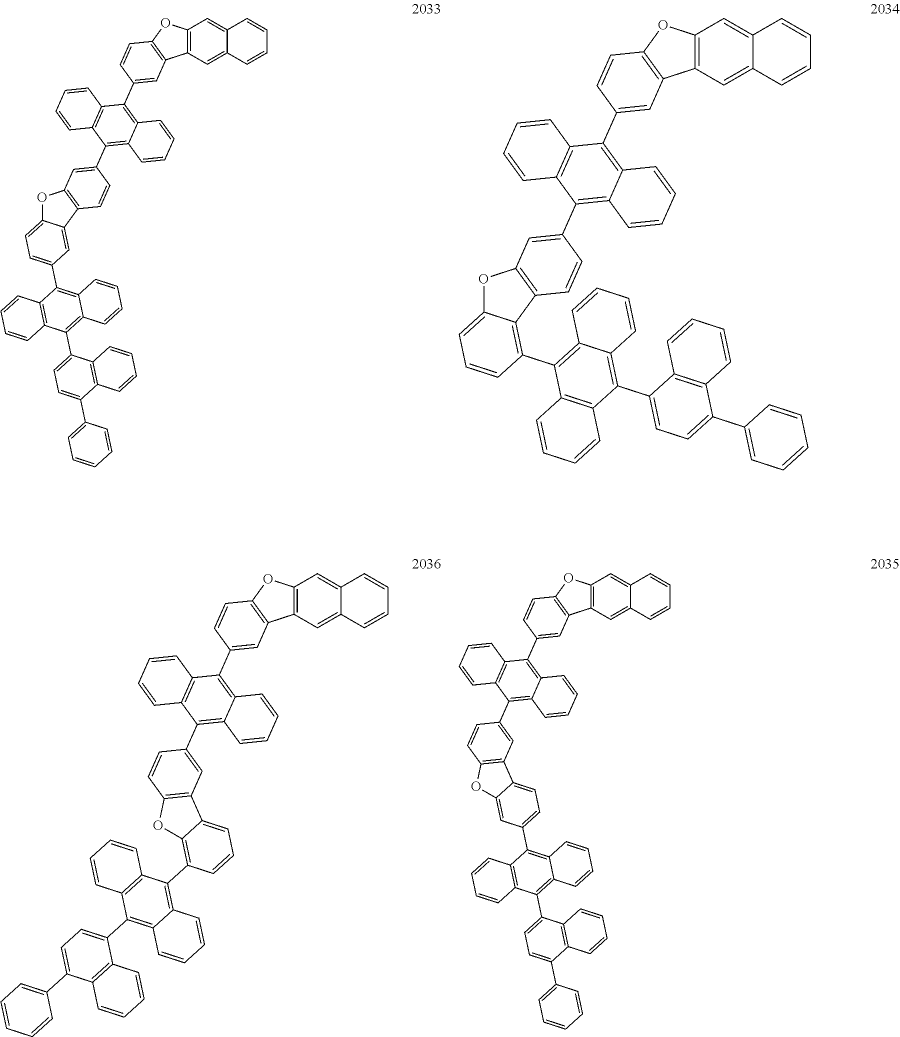

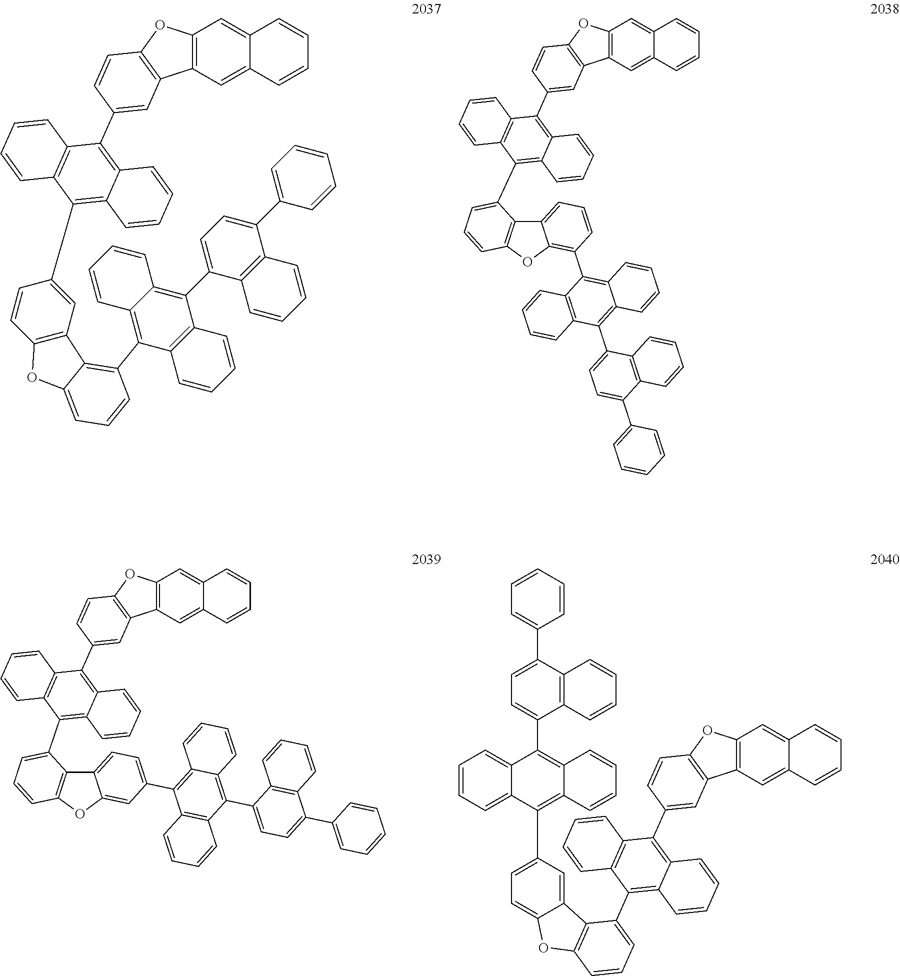

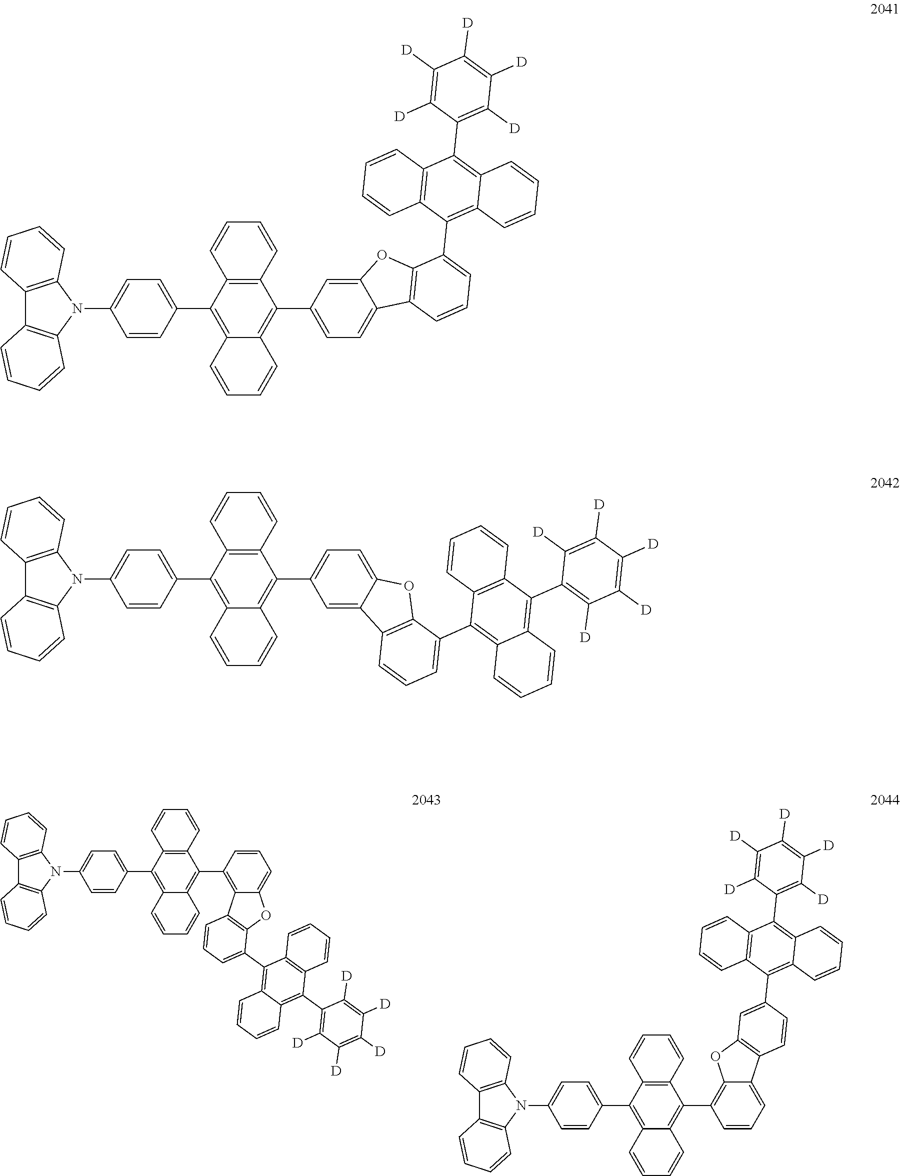

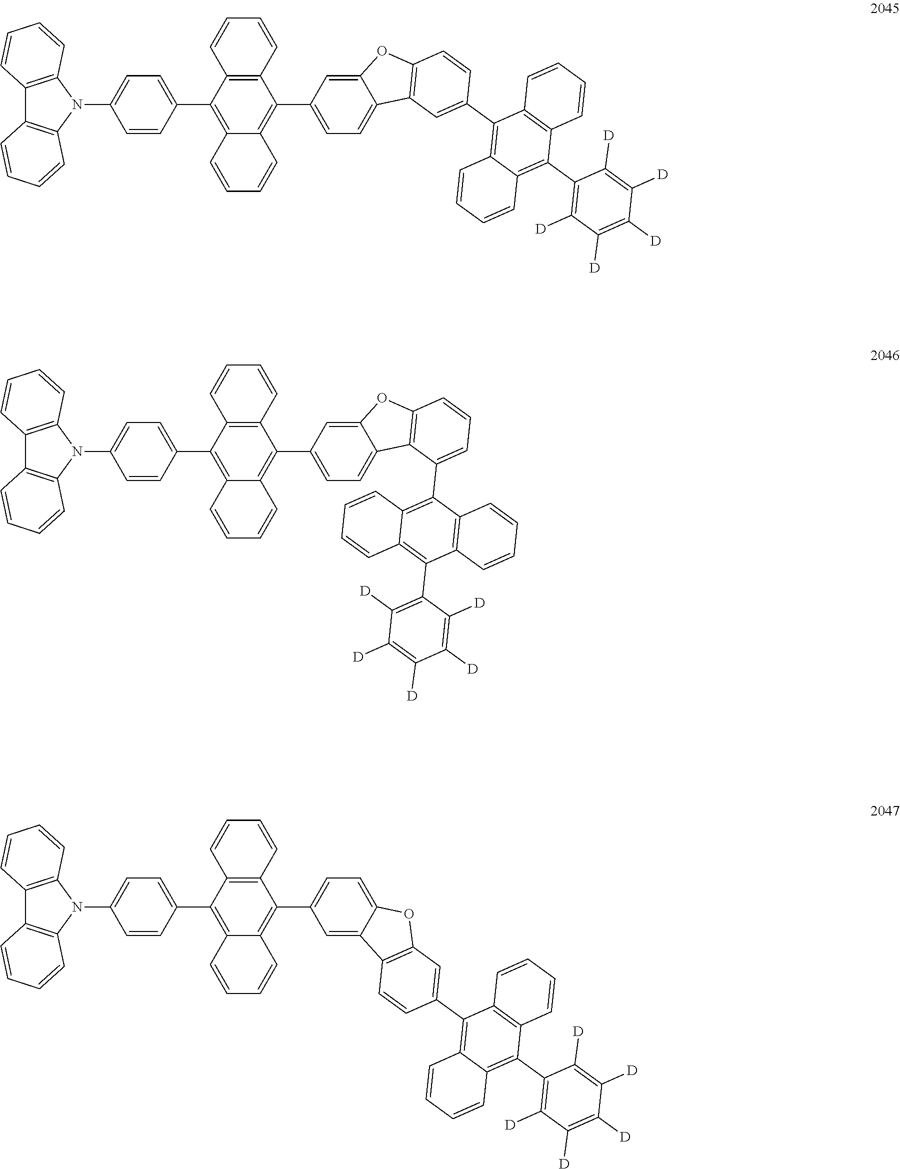 [SMILES: [2H]c1c([2H])c([2H])c(-c2c3ccccc3c(-c3ccc4oc5cc(-c6c7ccccc7c(-c7ccc(-n8c9ccccc9c9ccccc98)cc7)c7ccccc67)ccc5c4c3)c3ccccc23)c([2H])c1[2H] ; [2H]c1c([2H])c([2H])c(-c2c3ccccc3c(-c3cccc4oc5cc(-c6c7ccccc7c(-c7ccc(-n8c9ccccc9c9ccccc98)cc7)c7ccccc67)ccc5c34)c3ccccc23)c([2H])c1[2H] ; [2H]c1c([2H])c([2H])c(-c2c3ccccc3c(-c3ccc4c(c3)oc3ccc(-c5c6ccccc6c(-c6ccc(-n7c8ccccc8c8ccccc87)cc6)c6ccccc56)cc34)c3ccccc23)c([2H])c1[2H]]

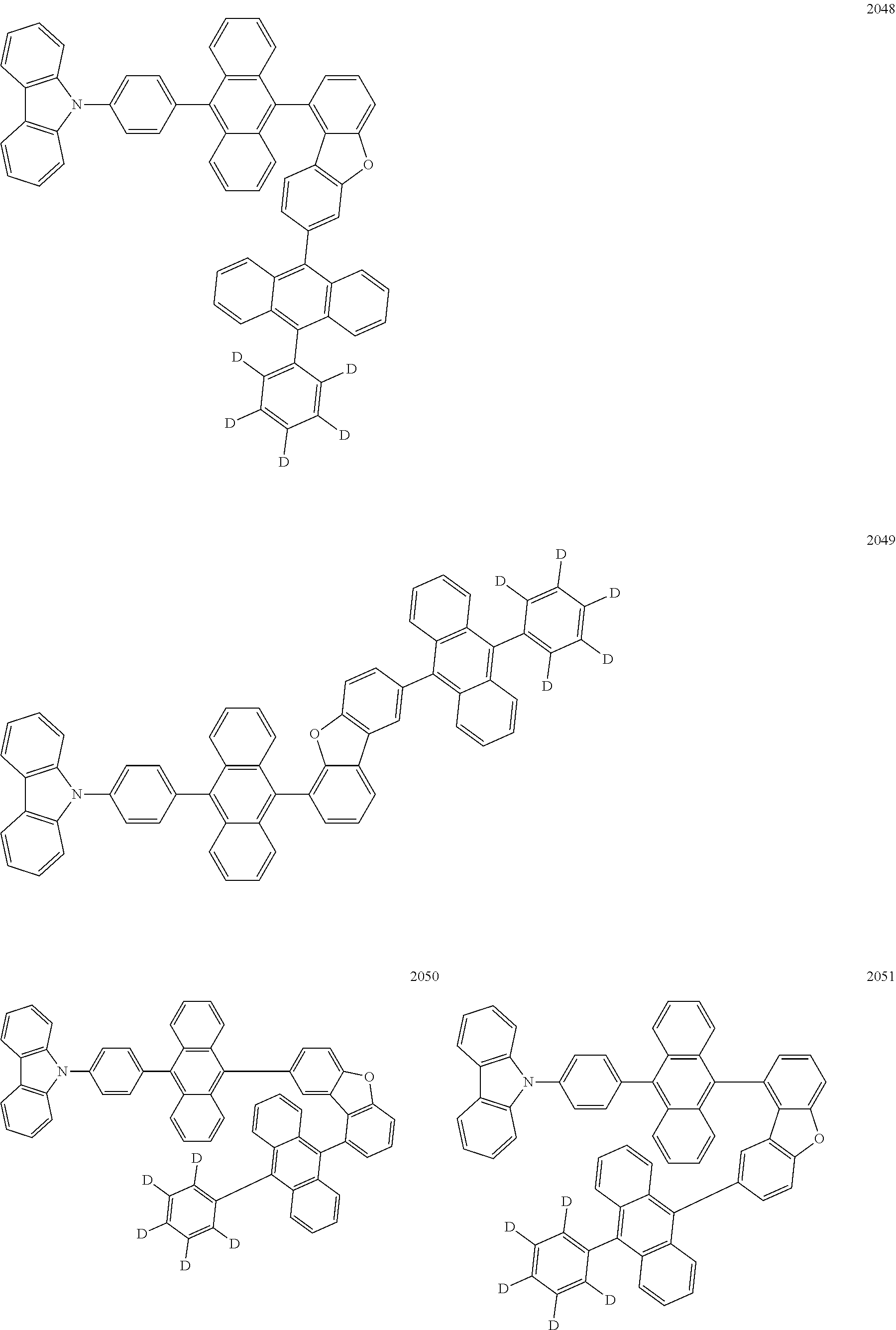

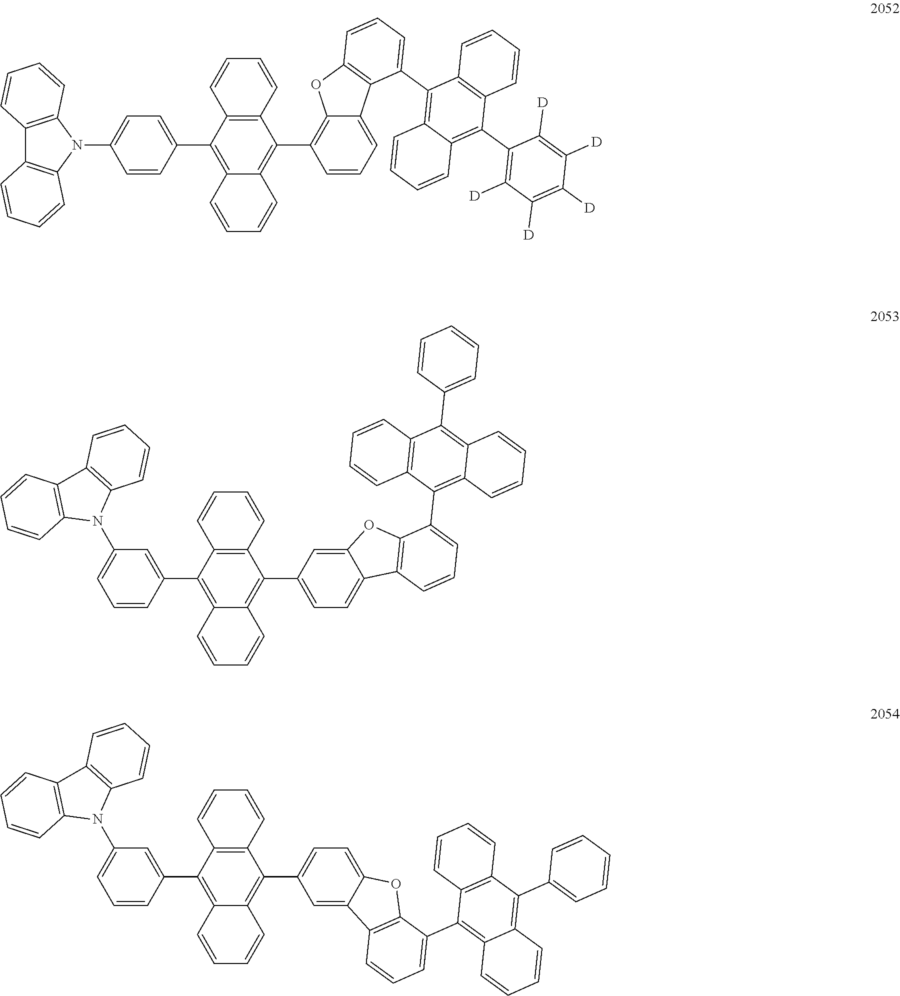 [SMILES: [2H]c1c([2H])c([2H])c(-c2c3ccccc3c(-c3cccc4oc5c(-c6c7ccccc7c(-c7ccc(-n8c9ccccc9c9ccccc98)cc7)c7ccccc67)cccc5c34)c3ccccc23)c([2H])c1[2H] ; c1ccc(-c2c3ccccc3c(-c3cccc4c3oc3cc(-c5c6ccccc6c(-c6cccc(-n7c8ccccc8c8ccccc87)c6)c6ccccc56)ccc34)c3ccccc23)cc1 ; c1ccc(-c2c3ccccc3c(-c3cccc4c3oc3ccc(-c5c6ccccc6c(-c6cccc(-n7c8ccccc8c8ccccc87)c6)c6ccccc56)cc34)c3ccccc23)cc1]

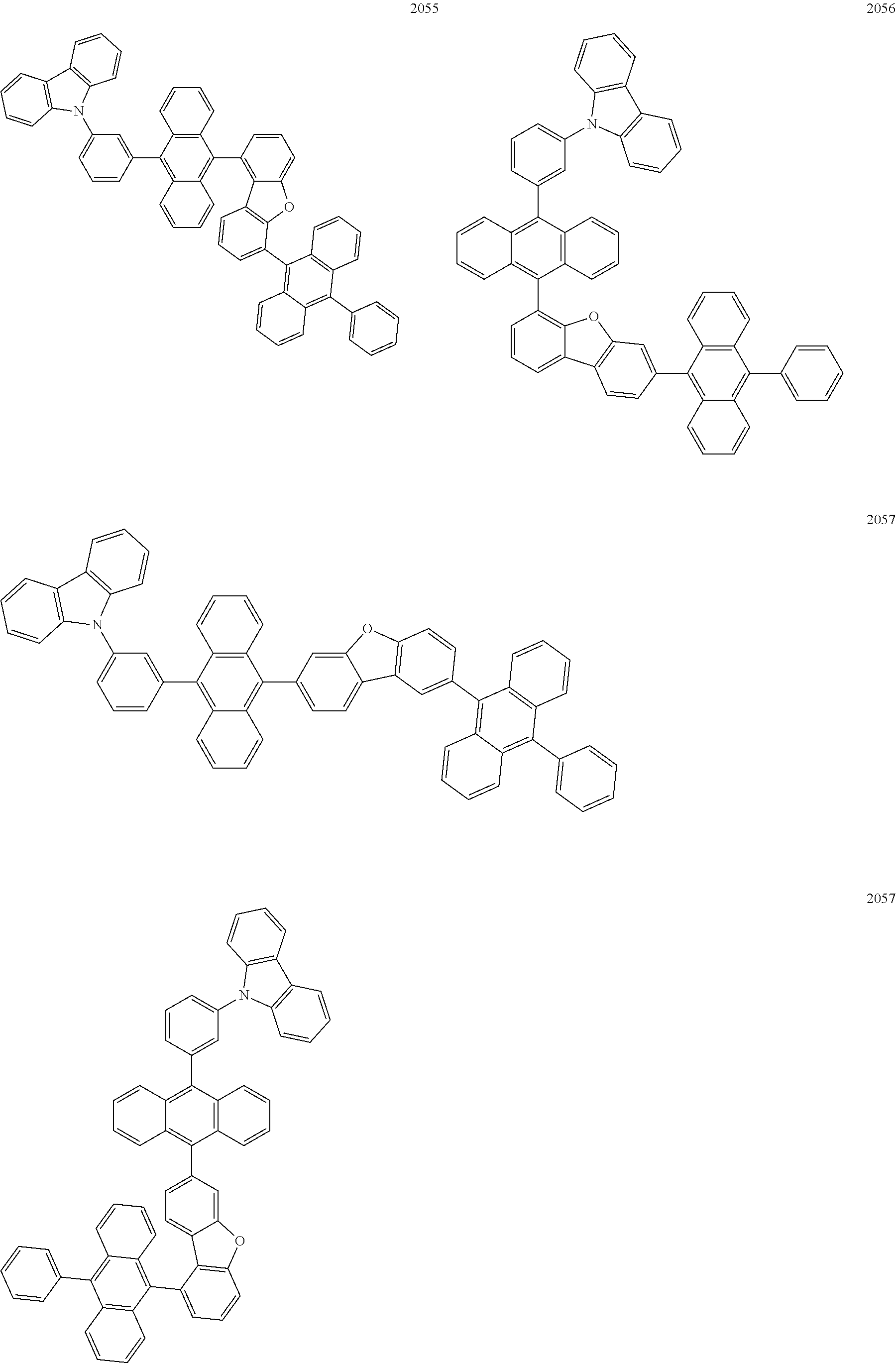 [SMILES: c1ccc(-c2c3ccccc3c(-c3cccc4c3oc3cccc(-c5c6ccccc6c(-c6cccc(-n7c8ccccc8c8ccccc87)c6)c6ccccc56)c34)c3ccccc23)cc1 ; c1ccc(-c2c3ccccc3c(-c3ccc4c(c3)oc3c(-c5c6ccccc6c(-c6cccc(-n7c8ccccc8c8ccccc87)c6)c6ccccc56)cccc34)c3ccccc23)cc1 ; c1ccc(-c2c3ccccc3c(-c3ccc4oc5cc(-c6c7ccccc7c(-c7cccc(-n8c9ccccc9c9ccccc98)c7)c7ccccc67)ccc5c4c3)c3ccccc23)cc1 ; c1ccc(-c2c3ccccc3c(-c3cccc4oc5cc(-c6c7ccccc7c(-c7cccc(-n8c9ccccc9c9ccccc98)c7)c7ccccc67)ccc5c34)c3ccccc23)cc1]

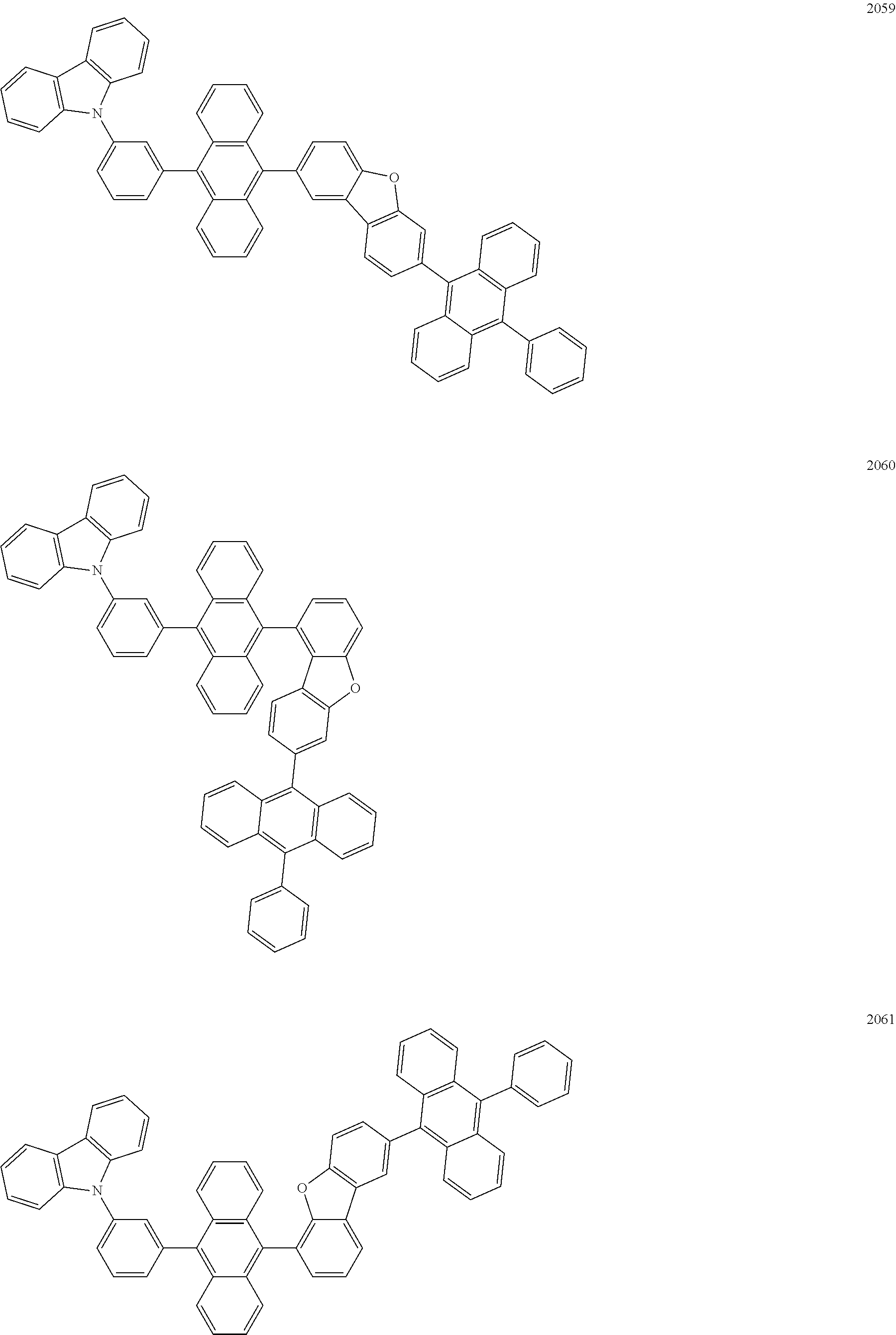 [SMILES: c1ccc(-c2c3ccccc3c(-c3ccc4c(c3)oc3ccc(-c5c6ccccc6c(-c6cccc(-n7c8ccccc8c8ccccc87)c6)c6ccccc56)cc34)c3ccccc23)cc1 ; c1ccc(-c2c3ccccc3c(-c3ccc4c(c3)oc3cccc(-c5c6ccccc6c(-c6cccc(-n7c8ccccc8c8ccccc87)c6)c6ccccc56)c34)c3ccccc23)cc1 ; c1ccc(-c2c3ccccc3c(-c3ccc4oc5c(-c6c7ccccc7c(-c7cccc(-n8c9ccccc9c9ccccc98)c7)c7ccccc67)cccc5c4c3)c3ccccc23)cc1]

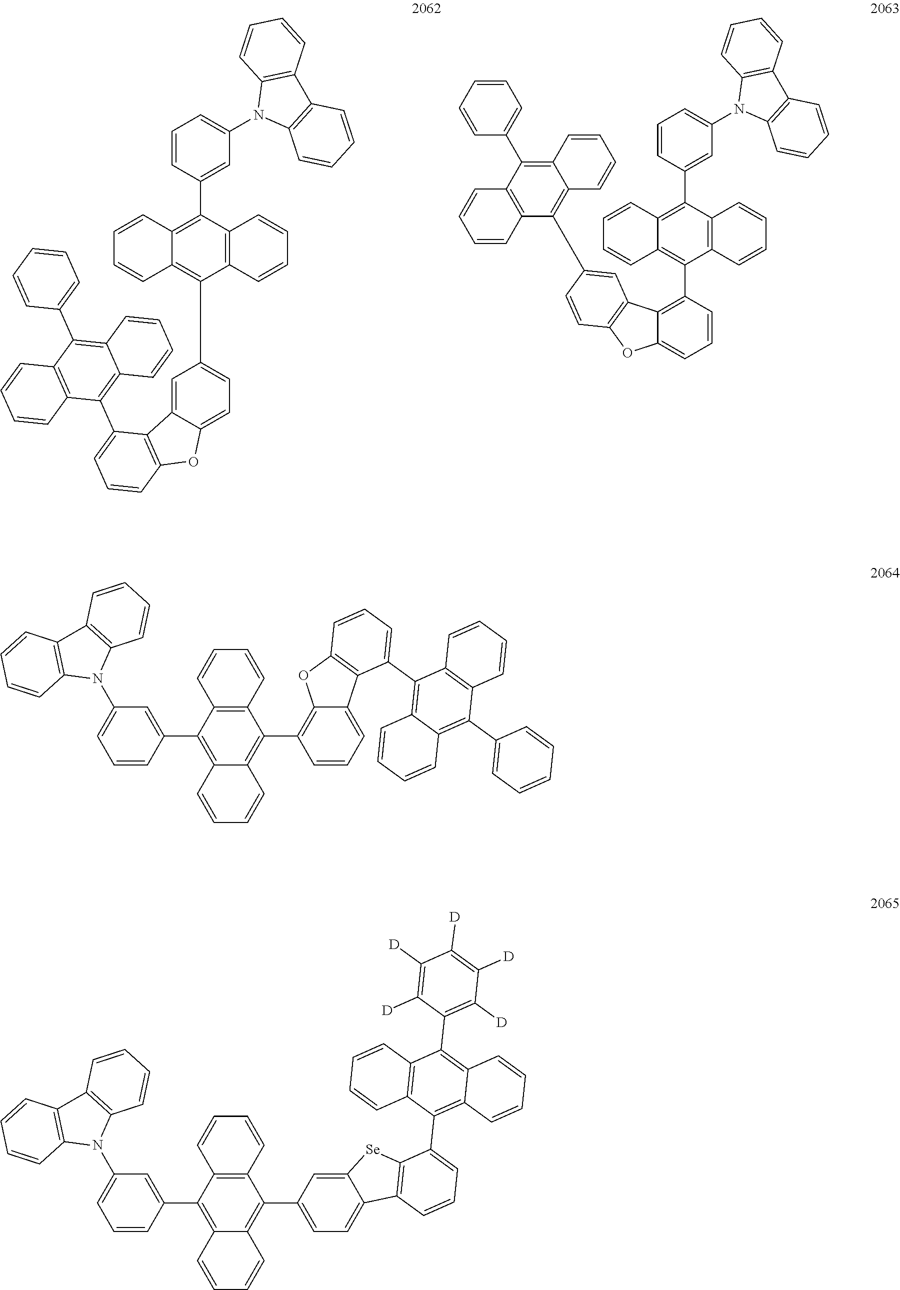 [SMILES: c1ccc(-c2c3ccccc3c(-c3cccc4oc5ccc(-c6c7ccccc7c(-c7cccc(-n8c9ccccc9c9ccccc98)c7)c7ccccc67)cc5c34)c3ccccc23)cc1 ; c1ccc(-c2c3ccccc3c(-c3ccc4oc5cccc(-c6c7ccccc7c(-c7cccc(-n8c9ccccc9c9ccccc98)c7)c7ccccc67)c5c4c3)c3ccccc23)cc1 ; c1ccc(-c2c3ccccc3c(-c3cccc4oc5c(-c6c7ccccc7c(-c7cccc(-n8c9ccccc9c9ccccc98)c7)c7ccccc67)cccc5c34)c3ccccc23)cc1 ; [2H]c1c([2H])c([2H])c(-c2c3ccccc3c(-c3cccc4c3[se]c3cc(-c5c6ccccc6c(-c6cccc(-n7c8ccccc8c8ccccc87)c6)c6ccccc56)ccc34)c3ccccc23)c([2H])c1[2H]]

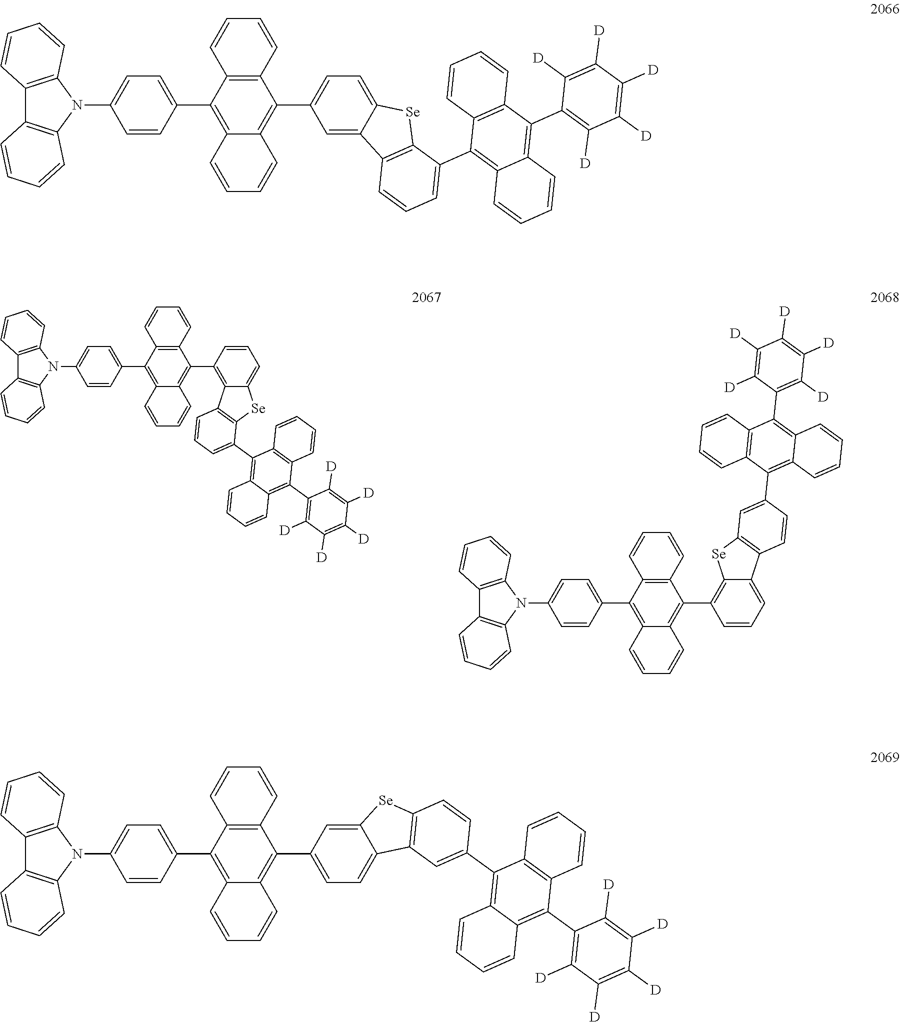

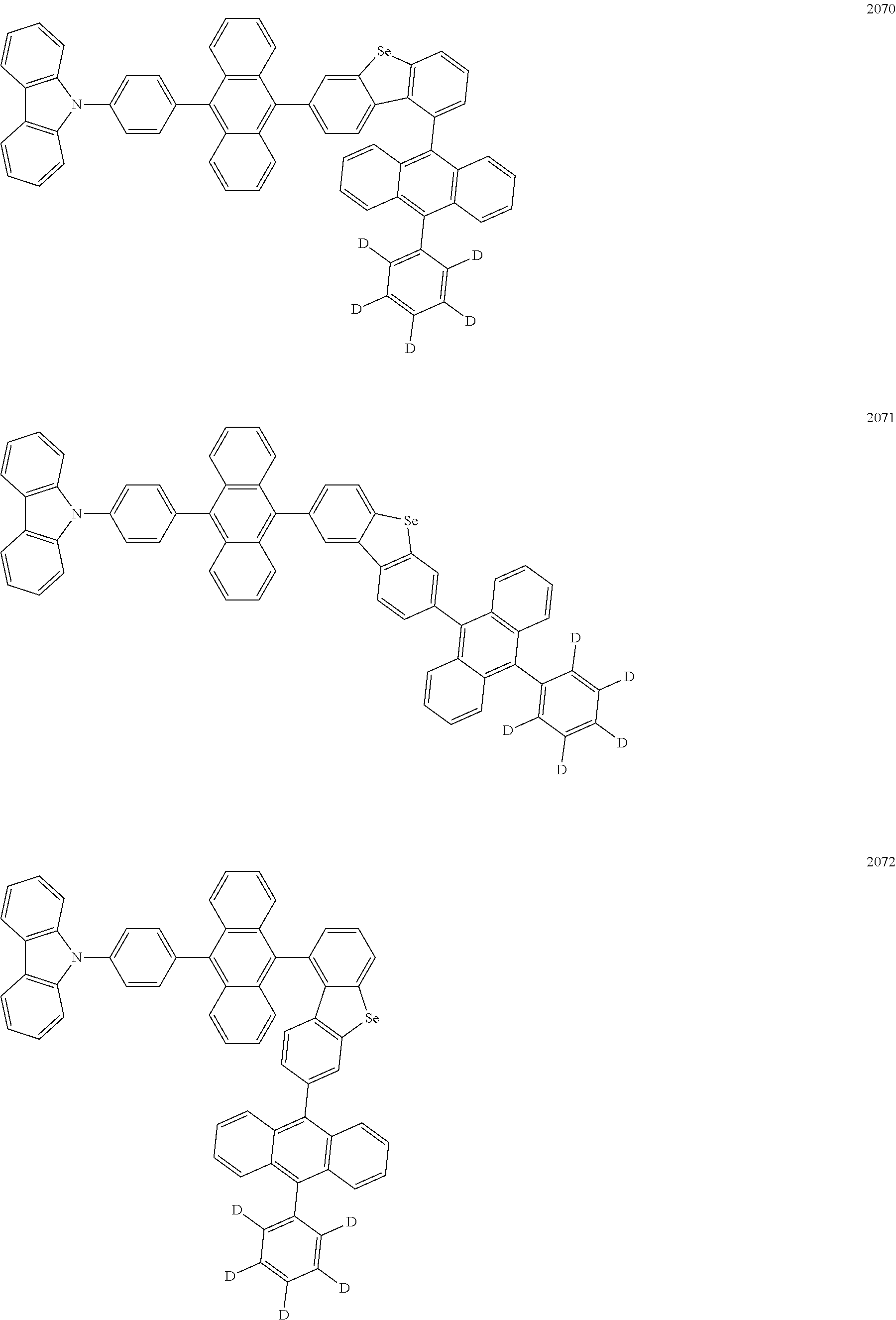 [SMILES: [2H]c1c([2H])c([2H])c(-c2c3ccccc3c(-c3cccc4[se]c5cc(-c6c7ccccc7c(-c7ccc(-n8c9ccccc9c9ccccc98)cc7)c7ccccc67)ccc5c34)c3ccccc23)c([2H])c1[2H] ; [2H]c1c([2H])c([2H])c(-c2c3ccccc3c(-c3ccc4c(c3)[se]c3ccc(-c5c6ccccc6c(-c6ccc(-n7c8ccccc8c8ccccc87)cc6)c6ccccc56)cc34)c3ccccc23)c([2H])c1[2H] ; [2H]c1c([2H])c([2H])c(-c2c3ccccc3c(-c3ccc4c(c3)[se]c3cccc(-c5c6ccccc6c(-c6ccc(-n7c8ccccc8c8ccccc87)cc6)c6ccccc56)c34)c3ccccc23)c([2H])c1[2H]]

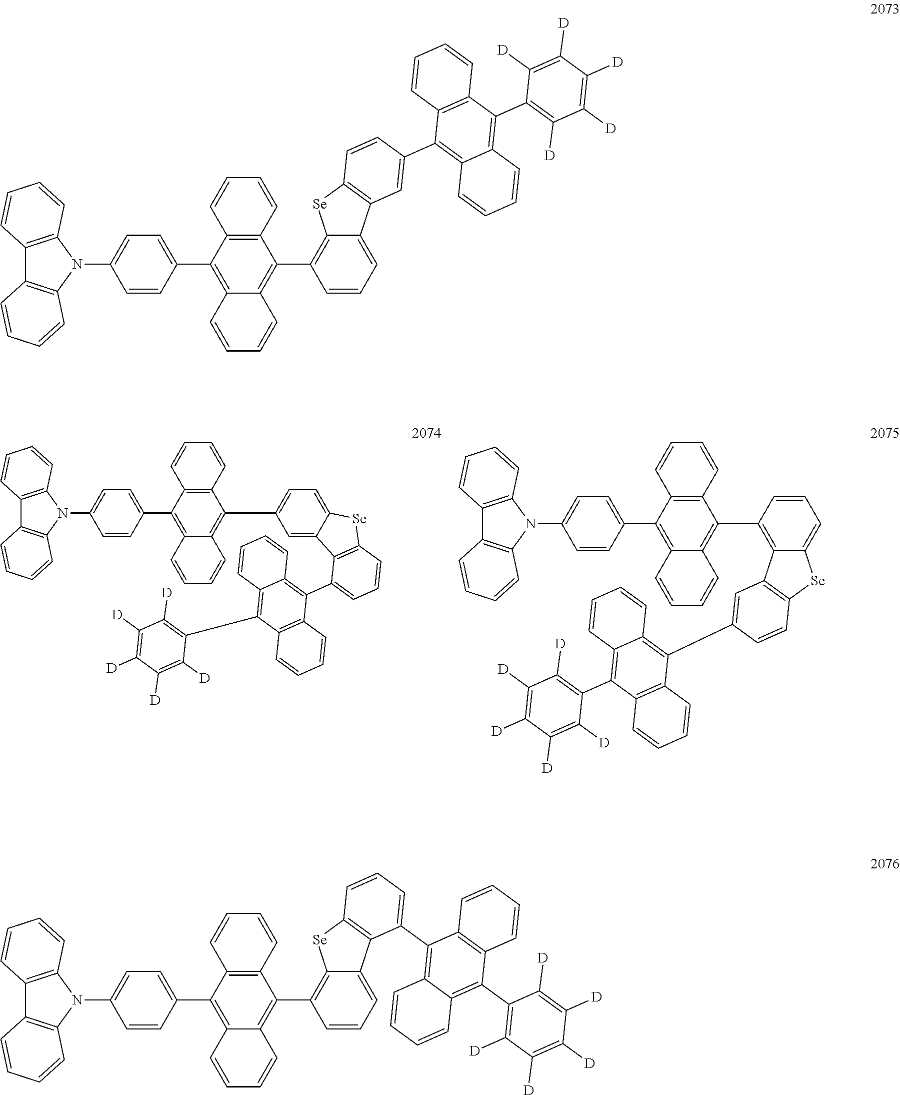

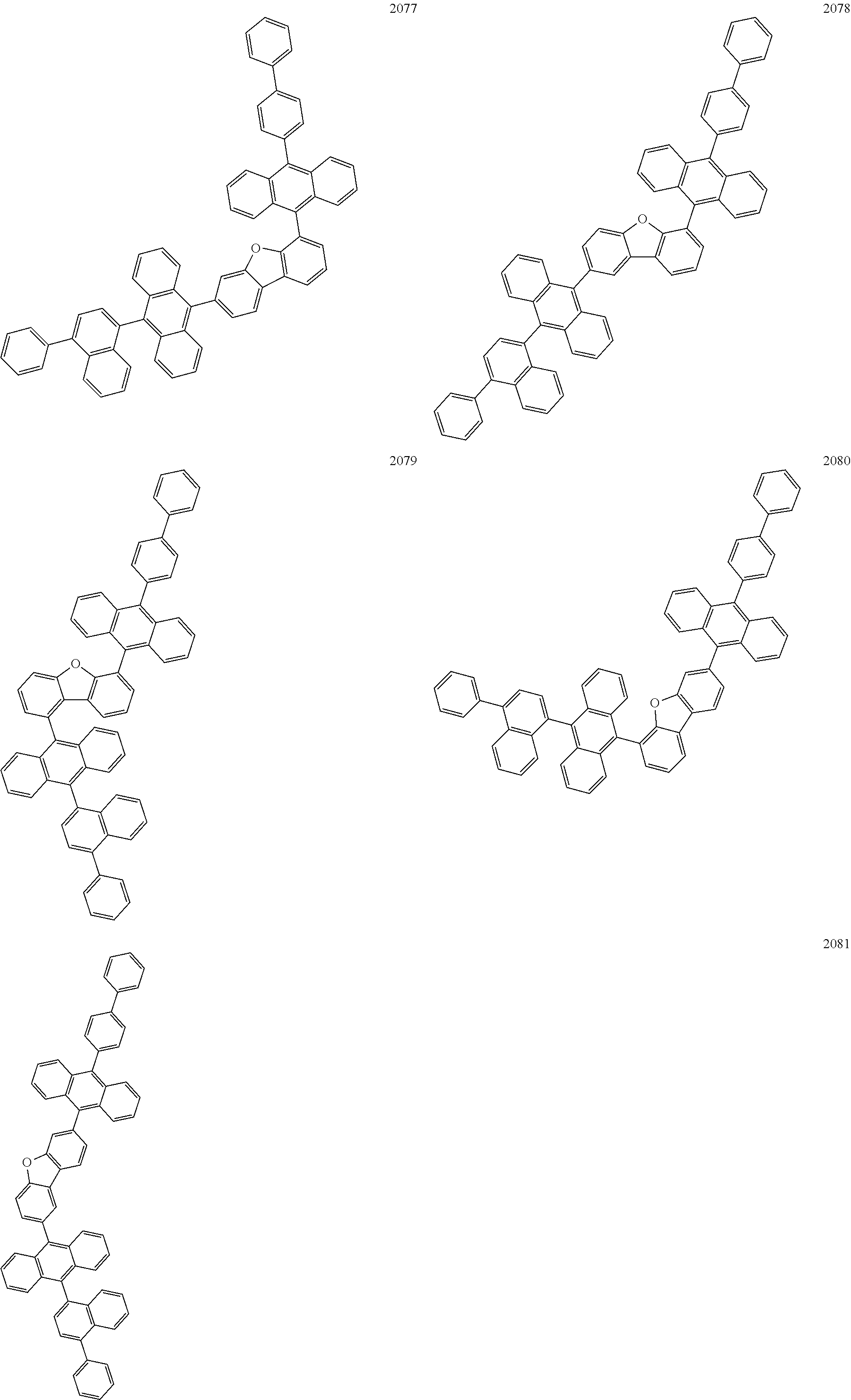

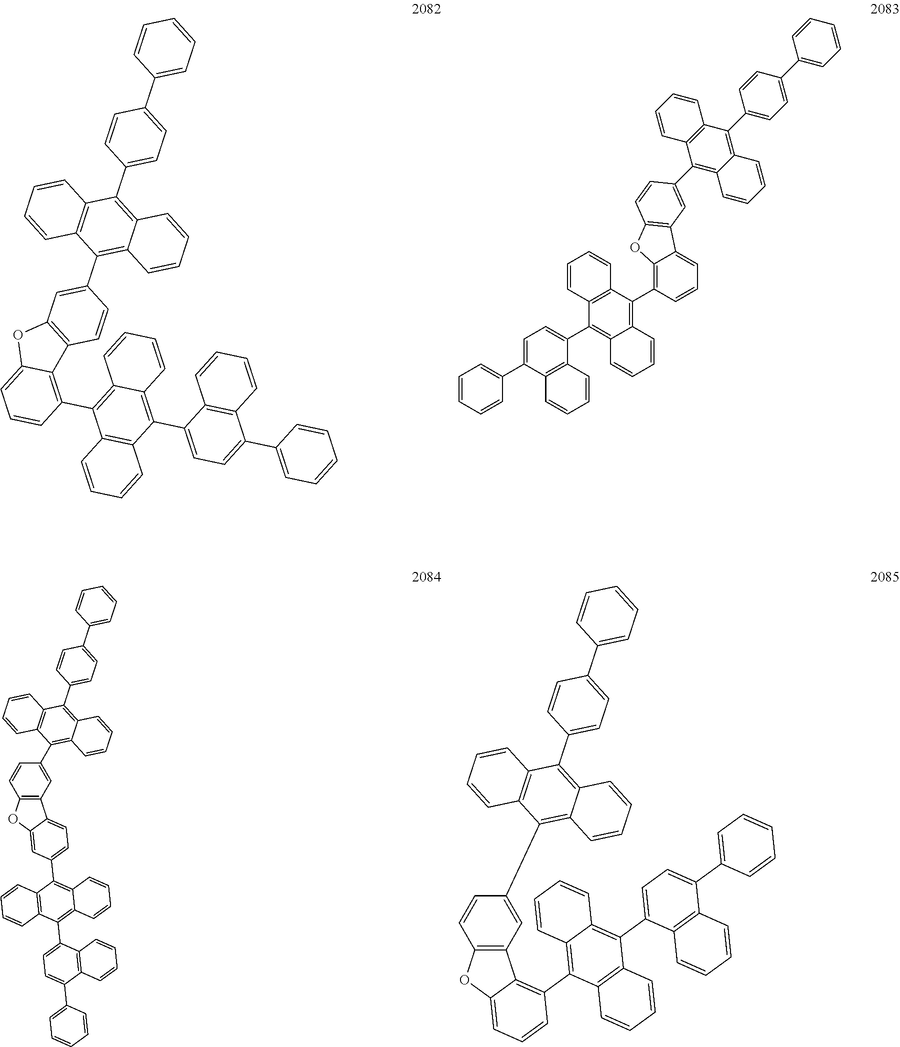 [SMILES: c1ccc(-c2ccc(-c3c4ccccc4c(-c4ccc5c(c4)oc4cccc(-c6c7ccccc7c(-c7ccc(-c8ccccc8)c8ccccc78)c7ccccc67)c45)c4ccccc34)cc2)cc1 ; c1ccc(-c2ccc(-c3c4ccccc4c(-c4ccc5oc6c(-c7c8ccccc8c(-c8ccc(-c9ccccc9)c9ccccc89)c8ccccc78)cccc6c5c4)c4ccccc34)cc2)cc1 ; c1ccc(-c2ccc(-c3c4ccccc4c(-c4ccc5oc6cc(-c7c8ccccc8c(-c8ccc(-c9ccccc9)c9ccccc89)c8ccccc78)ccc6c5c4)c4ccccc34)cc2)cc1 ; c1ccc(-c2ccc(-c3c4ccccc4c(-c4ccc5oc6cccc(-c7c8ccccc8c(-c8ccc(-c9ccccc9)c9ccccc89)c8ccccc78)c6c5c4)c4ccccc34)cc2)cc1]

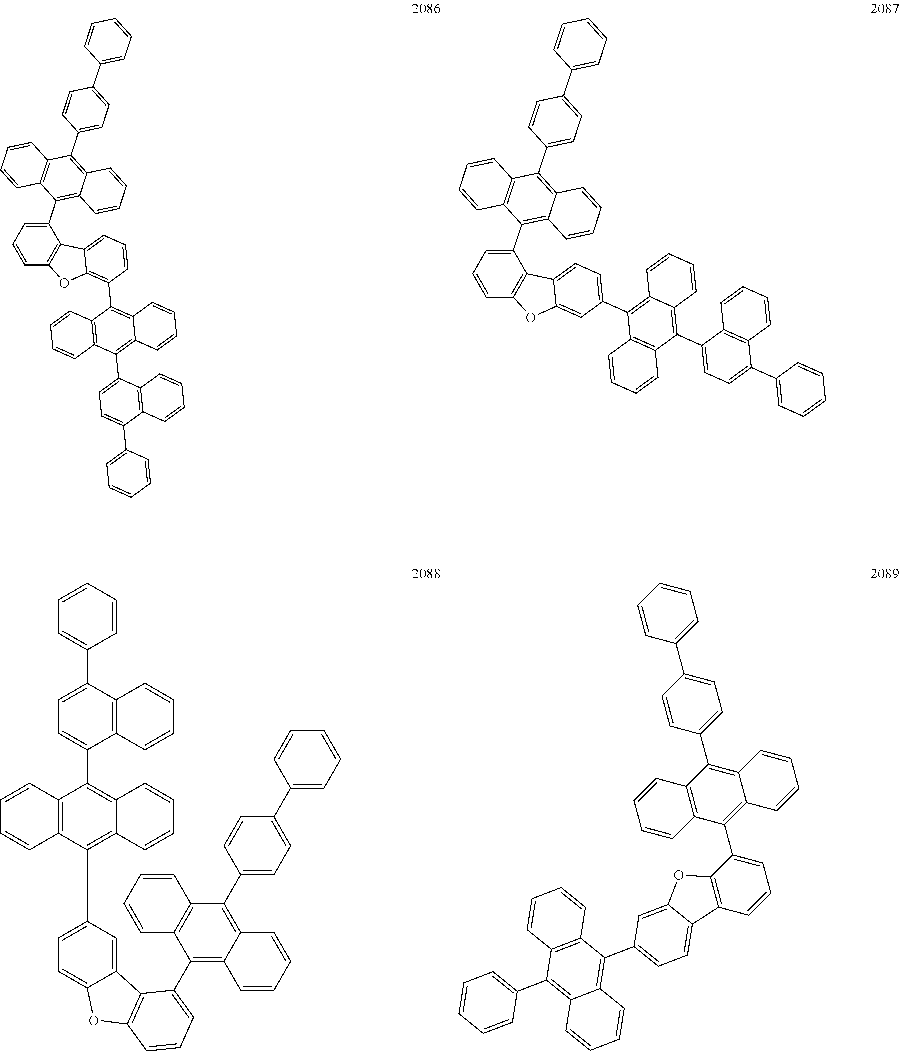 [SMILES: c1ccc(-c2ccc(-c3c4ccccc4c(-c4cccc5oc6c(-c7c8ccccc8c(-c8ccc(-c9ccccc9)c9ccccc89)c8ccccc78)cccc6c45)c4ccccc34)cc2)cc1 ; c1ccc(-c2ccc(-c3c4ccccc4c(-c4cccc5oc6cc(-c7c8ccccc8c(-c8ccc(-c9ccccc9)c9ccccc89)c8ccccc78)ccc6c45)c4ccccc34)cc2)cc1 ; c1ccc(-c2ccc(-c3c4ccccc4c(-c4cccc5oc6ccc(-c7c8ccccc8c(-c8ccc(-c9ccccc9)c9ccccc89)c8ccccc78)cc6c45)c4ccccc34)cc2)cc1 ; c1ccc(-c2ccc(-c3c4ccccc4c(-c4cccc5c4oc4cc(-c6c7ccccc7c(-c7ccccc7)c7ccccc67)ccc45)c4ccccc34)cc2)cc1]

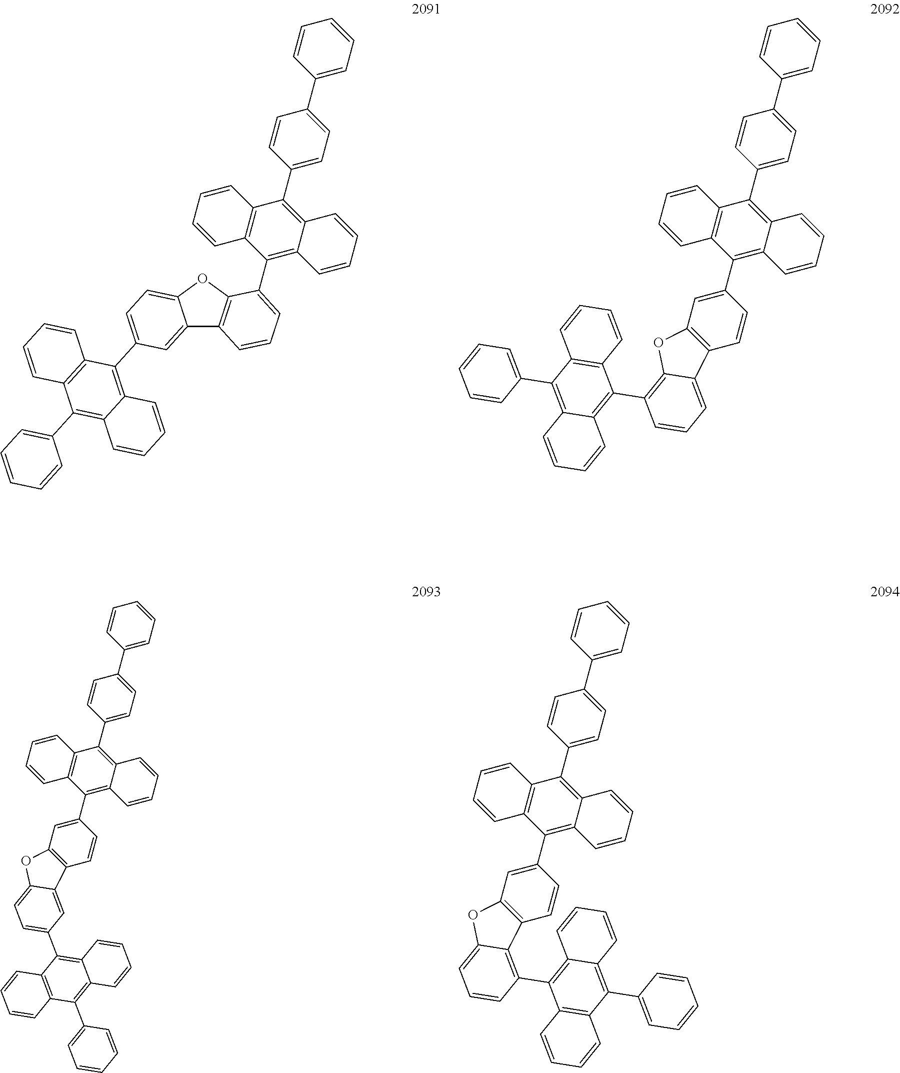 [SMILES: c1ccc(-c2ccc(-c3c4ccccc4c(-c4cccc5c4oc4ccc(-c6c7ccccc7c(-c7ccccc7)c7ccccc67)cc45)c4ccccc34)cc2)cc1 ; c1ccc(-c2ccc(-c3c4ccccc4c(-c4ccc5c(c4)oc4c(-c6c7ccccc7c(-c7ccccc7)c7ccccc67)cccc45)c4ccccc34)cc2)cc1 ; c1ccc(-c2ccc(-c3c4ccccc4c(-c4ccc5c(c4)oc4ccc(-c6c7ccccc7c(-c7ccccc7)c7ccccc67)cc45)c4ccccc34)cc2)cc1 ; c1ccc(-c2ccc(-c3c4ccccc4c(-c4ccc5c(c4)oc4cccc(-c6c7ccccc7c(-c7ccccc7)c7ccccc67)c45)c4ccccc34)cc2)cc1]

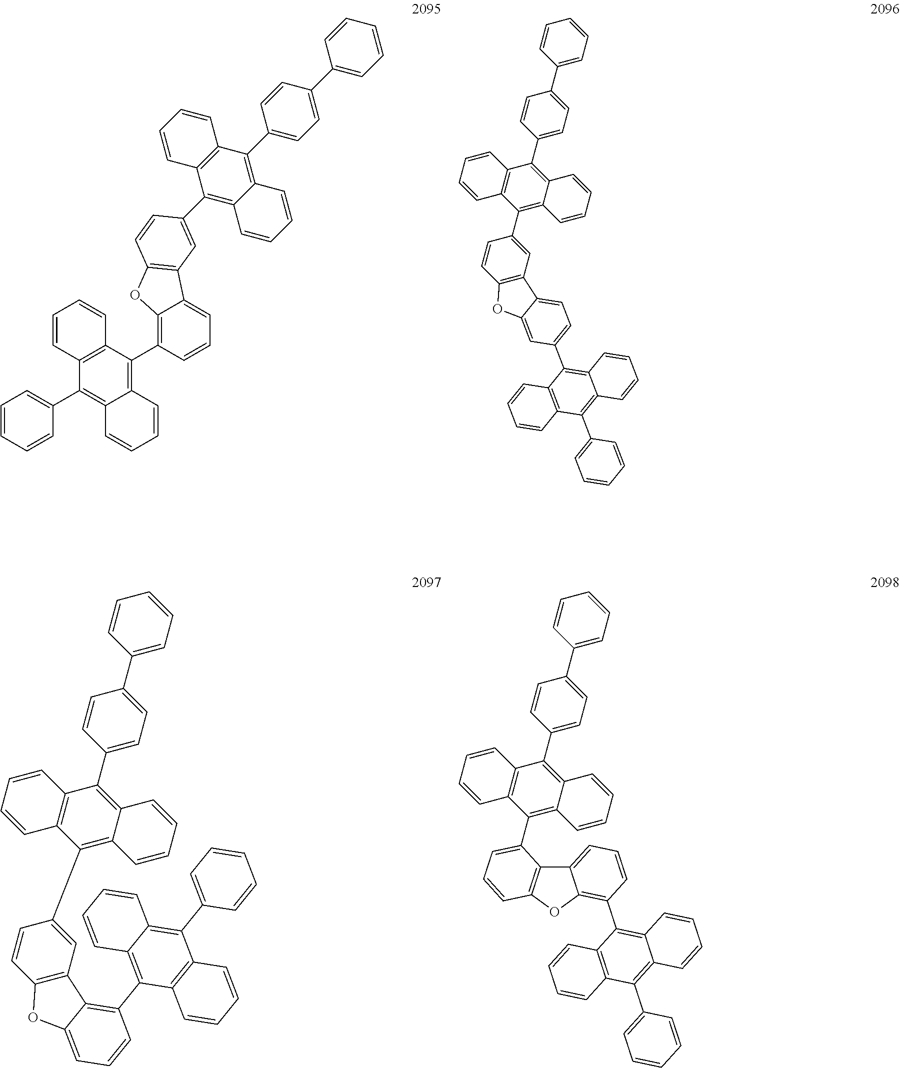 [SMILES: c1ccc(-c2ccc(-c3c4ccccc4c(-c4ccc5oc6c(-c7c8ccccc8c(-c8ccccc8)c8ccccc78)cccc6c5c4)c4ccccc34)cc2)cc1 ; c1ccc(-c2ccc(-c3c4ccccc4c(-c4ccc5oc6cc(-c7c8ccccc8c(-c8ccccc8)c8ccccc78)ccc6c5c4)c4ccccc34)cc2)cc1 ; c1ccc(-c2ccc(-c3c4ccccc4c(-c4ccc5oc6cccc(-c7c8ccccc8c(-c8ccccc8)c8ccccc78)c6c5c4)c4ccccc34)cc2)cc1 ; c1ccc(-c2ccc(-c3c4ccccc4c(-c4cccc5oc6c(-c7c8ccccc8c(-c8ccccc8)c8ccccc78)cccc6c45)c4ccccc34)cc2)cc1]

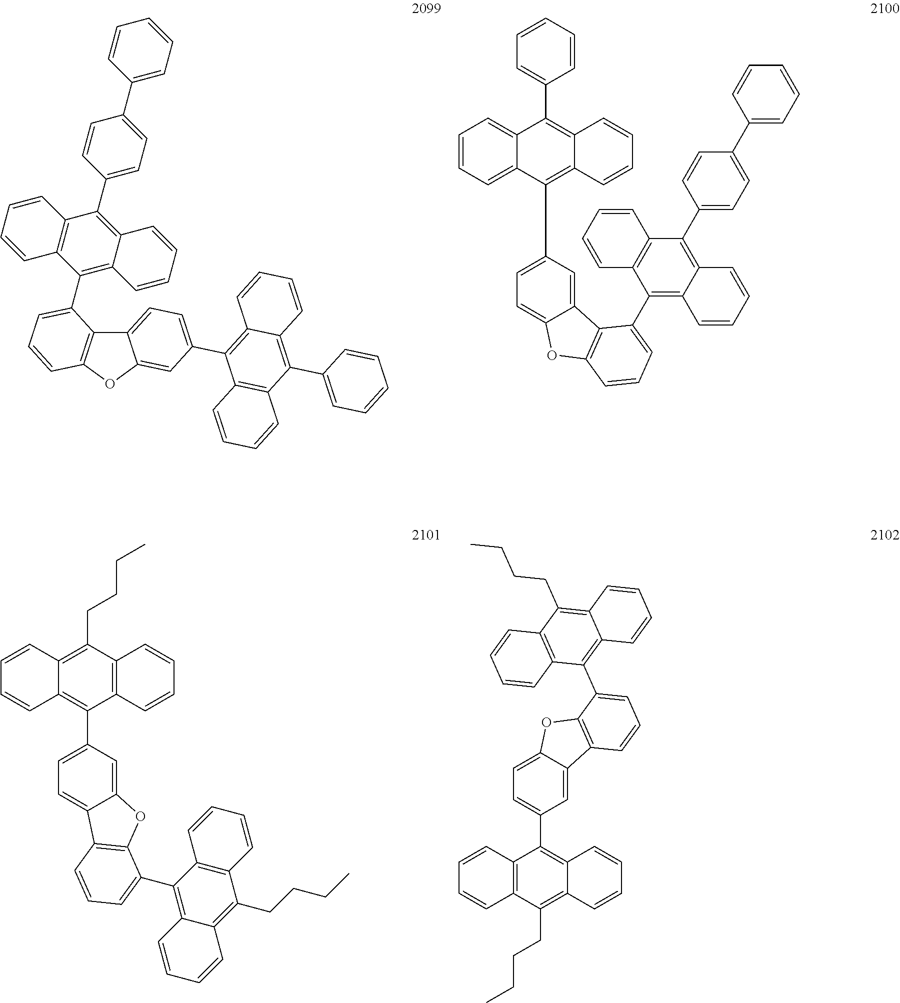 [SMILES: c1ccc(-c2ccc(-c3c4ccccc4c(-c4cccc5oc6cc(-c7c8ccccc8c(-c8ccccc8)c8ccccc78)ccc6c45)c4ccccc34)cc2)cc1 ; c1ccc(-c2ccc(-c3c4ccccc4c(-c4cccc5oc6ccc(-c7c8ccccc8c(-c8ccccc8)c8ccccc78)cc6c45)c4ccccc34)cc2)cc1 ; CCCCc1c2ccccc2c(-c2ccc3c(c2)oc2c(-c4c5ccccc5c(CCCC)c5ccccc45)cccc23)c2ccccc12 ; CCCCc1c2ccccc2c(-c2ccc3oc4c(-c5c6ccccc6c(CCCC)c6ccccc56)cccc4c3c2)c2ccccc12]

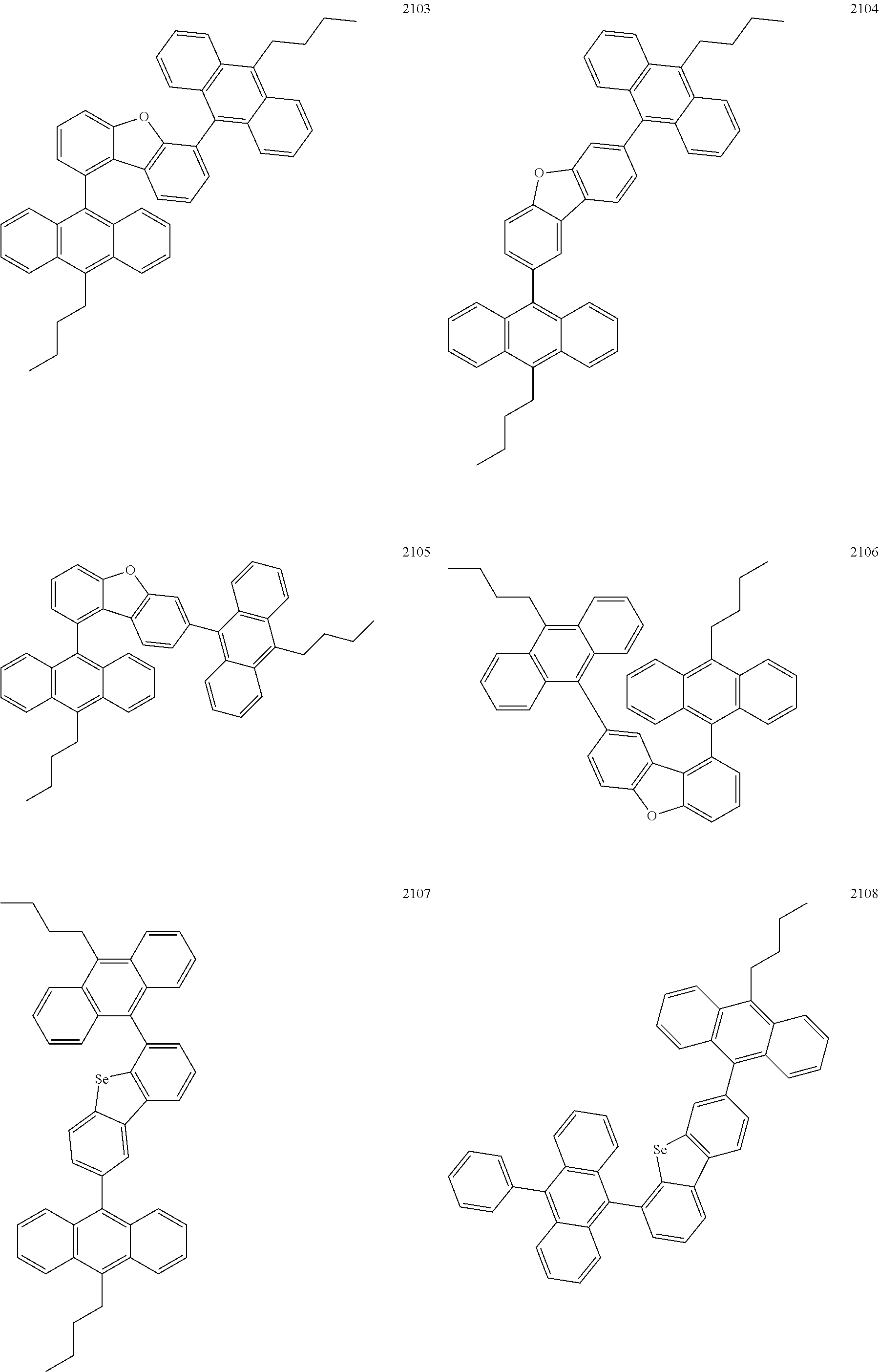 [SMILES: CCCCc1c2ccccc2c(-c2cccc3c2oc2cccc(-c4c5ccccc5c(CCCC)c5ccccc45)c23)c2ccccc12 ; CCCCc1c2ccccc2c(-c2ccc3c(c2)oc2ccc(-c4c5ccccc5c(CCCC)c5ccccc45)cc23)c2ccccc12 ; CCCCc1c2ccccc2c(-c2ccc3c(c2)oc2cccc(-c4c5ccccc5c(CCCC)c5ccccc45)c23)c2ccccc12 ; CCCCc1c2ccccc2c(-c2ccc3oc4cccc(-c5c6ccccc6c(CCCC)c6ccccc56)c4c3c2)c2ccccc12 ; CCCCc1c2ccccc2c(-c2ccc3[se]c4c(-c5c6ccccc6c(CCCC)c6ccccc56)cccc4c3c2)c2ccccc12 ; CCCCc1c2ccccc2c(-c2ccc3c(c2)[se]c2c(-c4c5ccccc5c(-c5ccccc5)c5ccccc45)cccc23)c2ccccc12]

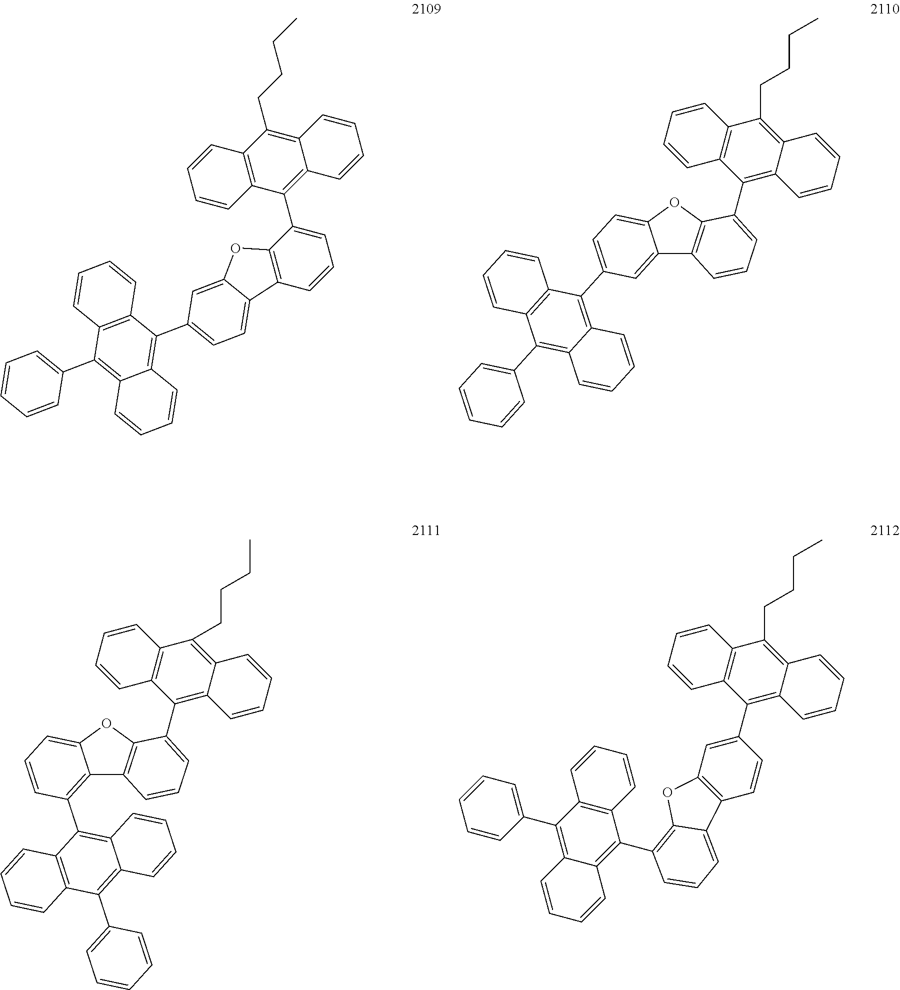 [SMILES: CCCCc1c2ccccc2c(-c2cccc3c2oc2cc(-c4c5ccccc5c(-c5ccccc5)c5ccccc45)ccc23)c2ccccc12 ; CCCCc1c2ccccc2c(-c2cccc3c2oc2ccc(-c4c5ccccc5c(-c5ccccc5)c5ccccc45)cc23)c2ccccc12 ; CCCCc1c2ccccc2c(-c2cccc3c2oc2cccc(-c4c5ccccc5c(-c5ccccc5)c5ccccc45)c23)c2ccccc12 ; CCCCc1c2ccccc2c(-c2ccc3c(c2)oc2c(-c4c5ccccc5c(-c5ccccc5)c5ccccc45)cccc23)c2ccccc12]

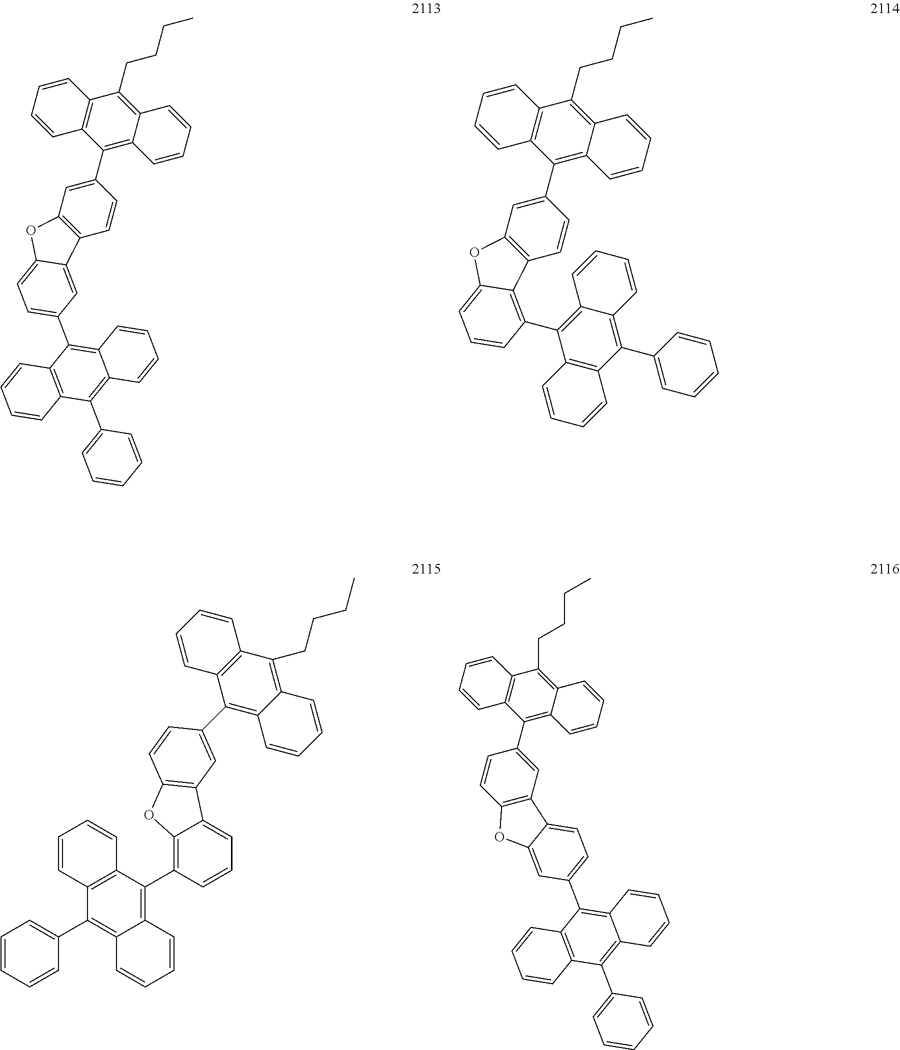 [SMILES: CCCCc1c2ccccc2c(-c2ccc3c(c2)oc2ccc(-c4c5ccccc5c(-c5ccccc5)c5ccccc45)cc23)c2ccccc12 ; CCCCc1c2ccccc2c(-c2ccc3c(c2)oc2cccc(-c4c5ccccc5c(-c5ccccc5)c5ccccc45)c23)c2ccccc12 ; CCCCc1c2ccccc2c(-c2ccc3oc4c(-c5c6ccccc6c(-c6ccccc6)c6ccccc56)cccc4c3c2)c2ccccc12 ; CCCCc1c2ccccc2c(-c2ccc3oc4cc(-c5c6ccccc6c(-c6ccccc6)c6ccccc56)ccc4c3c2)c2ccccc12]

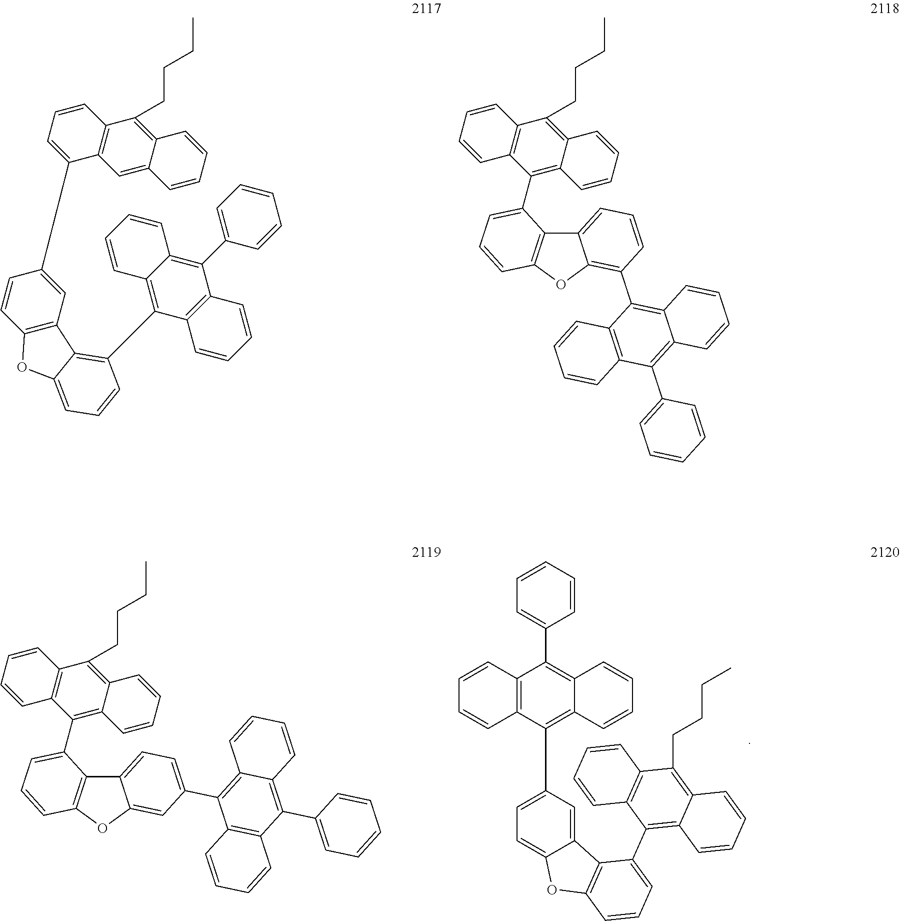 [SMILES: CCCCc1c2ccccc2cc2c(-c3ccc4oc5cccc(-c6c7ccccc7c(-c7ccccc7)c7ccccc67)c5c4c3)cccc12 ; CCCCc1c2ccccc2c(-c2cccc3oc4c(-c5c6ccccc6c(-c6ccccc6)c6ccccc56)cccc4c23)c2ccccc12 ; CCCCc1c2ccccc2c(-c2cccc3oc4cc(-c5c6ccccc6c(-c6ccccc6)c6ccccc56)ccc4c23)c2ccccc12 ; CCCCc1c2ccccc2c(-c2cccc3oc4ccc(-c5c6ccccc6c(-c6ccccc6)c6ccccc56)cc4c23)c2ccccc12]

When the host is a mixture of an electron-transporting host and a hole-transporting host, the weight ratio of the electron-transporting host and the hole-transporting host may be 1:9 to 9:1, for example, 2:8 to 8:2, for example, 4:6 to 6:4, for example, 5:5. When the weight ratio of the electron-transporting host and the hole-transporting host satisfies the above-described ranges, the hole-and-electron-transporting balance in the emission layer 15 may be made.

Dopant in Emission Layer 15

The dopant may include the heterocyclic compound.

Sensitizer in Emission Layer 15

In an embodiment, the sensitizer may include a phosphorescent sensitizer including at least one metal Period 1 transition metal, Period 2 transition metal, Period 3 transition metal, or a combination thereof.

In an embodiment, the sensitizer may include metal an organic ligand (L₁₁) including at least one metal (M₁₁) of a first-row transition metal of the Periodic Table of Elements, a second-row transition metal of the Periodic Table of Elements, a third-row transition metal of the Periodic Table of Elements, or a combination thereof, and L₁₁ and M₁₁ may form 1, 2, 3, or 4 cyclometalated rings.

In an embodiment, the sensitizer may include an organometallic compound represented by Formula 101:

M₁₁(L₁₁)ₙ₁₁(L₁₂)ₙ₁₂ Formula 101
wherein, in Formula 101,
M₁₁ may be a first-row transition metal of the Periodic Table of Elements, a second-row transition metal of the Periodic Table of Elements, a third-row transition metal of the Periodic Table of Elements, or a combination thereof;
may be a ligand represented by one of Formulae 1-1 to 1-4;
L₁₂ may be a monodentate ligand or a bidentate ligand;
n11 may be 1,
n12 may be 0, 1, or 2:

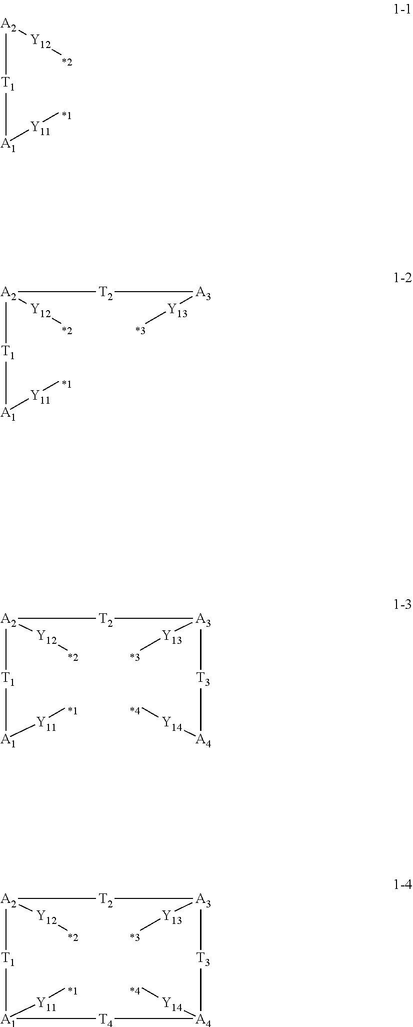

wherein, in Formulae 1-1 to 1-4,
A₁ to A₄ may each independently be a substituted or unsubstituted C₅-C₃₀ carbocyclic group, a substituted or unsubstituted C₁-C₃₀ heterocyclic group, or a non-cyclic group,
Y₁₁ to Y₁₄ may each independently be a chemical bond, O, S, N(R₉₁), B(R₉₁), P(R₉₁), or C(R₉₁)(R₉₂),
T₁ to T₄ may each independently be a single bond, a double bond, *—N(R₉₃)—*′, *—B(R₉₃)—′, *-1D(R₉₃)—′, *—C(R₉₃)(R₉₄)—′, *—Si(R₉₃)(R₉₄)—*′, *—Ge(R₉₃)(R₉₄)—′, *—S-′, *—Se-′, *—O-′, *—C(═O)—′, *—S(═O)—′, *—S(═O)₂-′, *—C(R₉₃)=′, *═C(R₉₃)—*′, *—C(R₉₃)═C(R₉₄)—*′, *—C(═S)—′, or *—C≡C-′,
a substituent of the substituted C₅-C₃₀ carbocyclic group, a substituent of substituted C₁-C₃₀ heterocyclic group, and R₉₁ to R₉₄ may each independently be hydrogen, deuterium, —F, —Cl, —Br, —I, —SF₅, a hydroxyl group, a cyano group, a nitro group, an amidino group, a hydrazine group, a hydrazone group, a carboxylic acid or a salt thereof, a sulfonic acid or a salt thereof, a phosphoric acid or a salt thereof, a substituted or unsubstituted C₁-C₆₀ alkyl group, a substituted or unsubstituted C₂-C₆₀ alkenyl group, a substituted or unsubstituted C₂-C₆₀ alkynyl group, a substituted or unsubstituted C₁-C₆₀ alkoxy group, a substituted or unsubstituted C₃-C₁₀ cycloalkyl group, a substituted or unsubstituted C₁-C₁₀ heterocycloalkyl group, a substituted or unsubstituted C₃-C₁₀ cycloalkenyl group, a substituted or unsubstituted C₂-C₁₀ heterocycloalkenyl group, a substituted or unsubstituted C₆-C₆₀ aryl group, a substituted or unsubstituted C₆-C₆₀ aryloxy group, a substituted or unsubstituted C₆-C₆₀ arylthio group, a substituted or unsubstituted C₁-C₆₀ heteroaryl group, a substituted or unsubstituted monovalent aromatic condensed polycyclic group, a substituted or unsubstituted monovalent aromatic condensed heteropolycyclic group, a substituted or unsubstituted monovalent non-aromatic condensed polycyclic group, a substituted or unsubstituted monovalent non-aromatic condensed heteropolycyclic group, —Si(Q₁)(Q₂)(Q₃), —Ge(Q₁)(Q₂)(Q₃), —B(Q₁)(Q₂), —N(Q₁)(Q₂), —P(Q₁)(Q₂), —C(═O)(Q₁), —S(═O)(Q₁), —S(═O)₂(Q₁), —P(═O)(Q₁)(Q₂), or —P(═S)(Q₁)(Q₂), wherein each of a substituent of the substituted C₅-C₃₀ carbocyclic group and a substituent of substituted C₁-C₃₀ heterocyclic group is not hydrogen,
*₁, *₂, *₃, and *₄ each indicate a binding site to M₁₁ and
Q₁ to Q₃ may each independently be hydrogen, deuterium, —F, —Cl, —Br, —I, a hydroxyl group, a cyano group, a nitro group, an amidino group, a hydrazine group, a hydrazone group, a C₁-C₆₀ alkyl group, a C₂-C₆₀ alkenyl group, a C₂-C₆₀ alkynyl group, a C₁-C₆₀ alkoxy group, a C₃-C₁₀ cycloalkyl group, a C₁-C₁₀ heterocycloalkyl group, a C₃-C₁₀ cycloalkenyl group, a C₂-C₁₀ heterocycloalkenyl group, a C₆-C₆₀ aryl group, a C₇-C₆₀ alkylaryl group, a C₆-C₆₀ aryloxy group, a C₆-C₆₀ arylthio group, a C₁-C₆₀ heteroaryl group, a C₂-C₆₀ heteroalkylaryl group, a C₁-C₆₀ heteroaryloxy group, a C₁-C₆₀ heteroarylthio group, a monovalent aromatic condensed polycyclic group, a monovalent aromatic condensed heteropolycyclic group, a monovalent non-aromatic condensed polycyclic group, a monovalent non-aromatic condensed heteropolycyclic group, a C₁-C₆₀ alkyl group that is substituted with at least one deuterium, —F, a cyano group, a C₁-C₆₀ alkyl group, a C₆-C₆₀ aryl group, or a combination thereof, or a C₆-C₆₀ aryl group that is substituted with deuterium, —F, a cyano group, a C₁-C₆₀ alkyl group, a C₆-C₆₀ aryl group, or a combination thereof.

In one or more embodiments, the sensitizer may be compounds of Groups I to VIII, but embodiments of the present disclosure are not limited thereto:

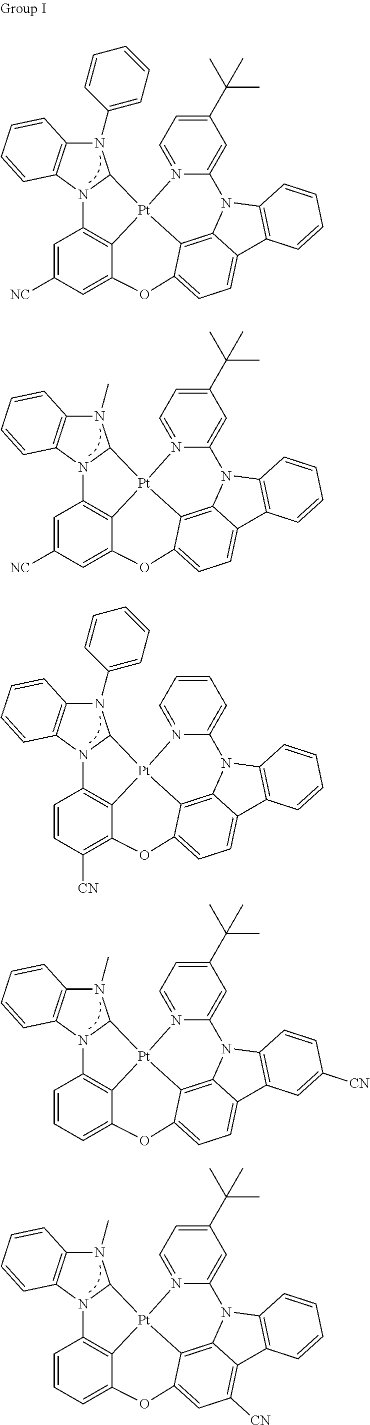

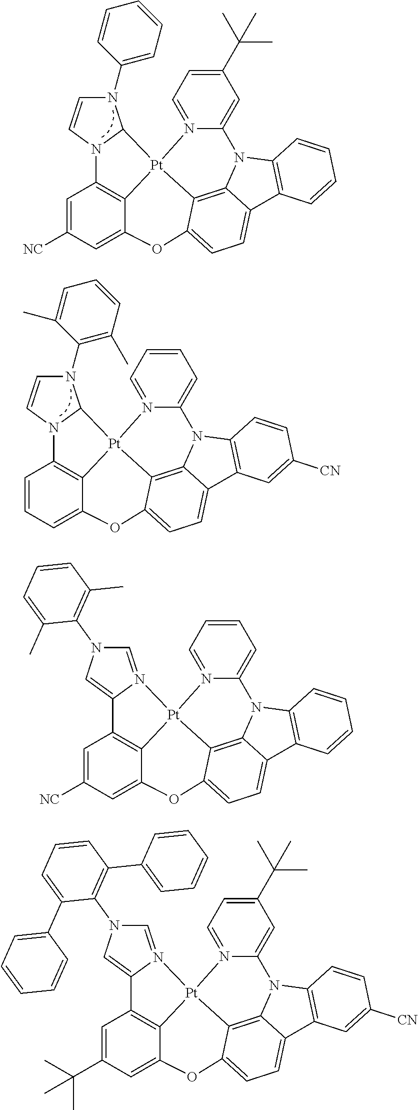

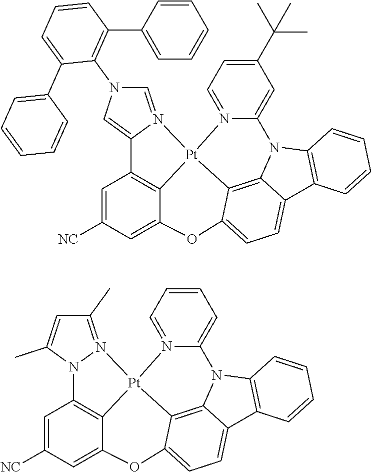

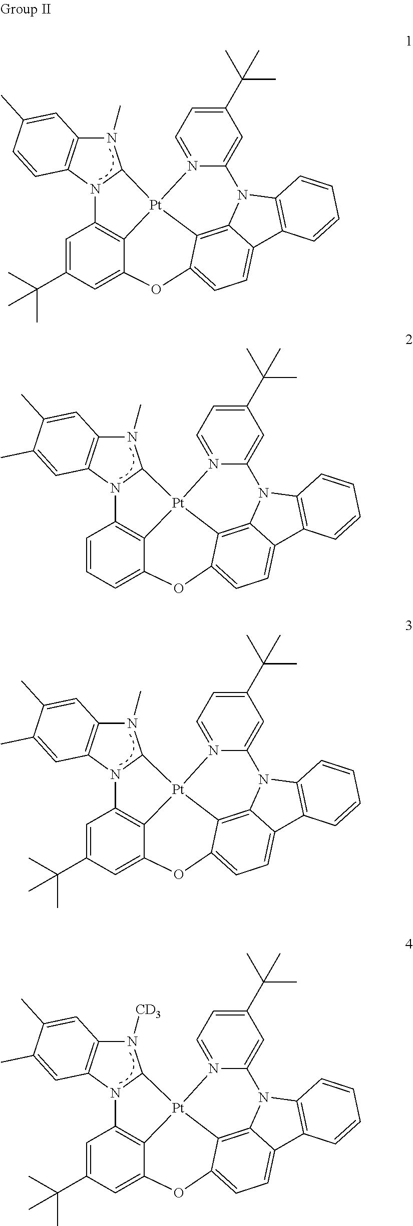

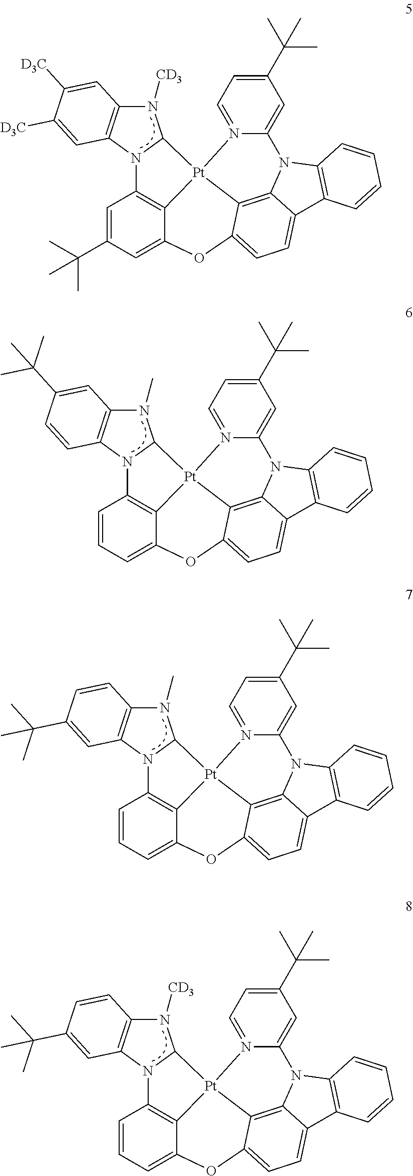

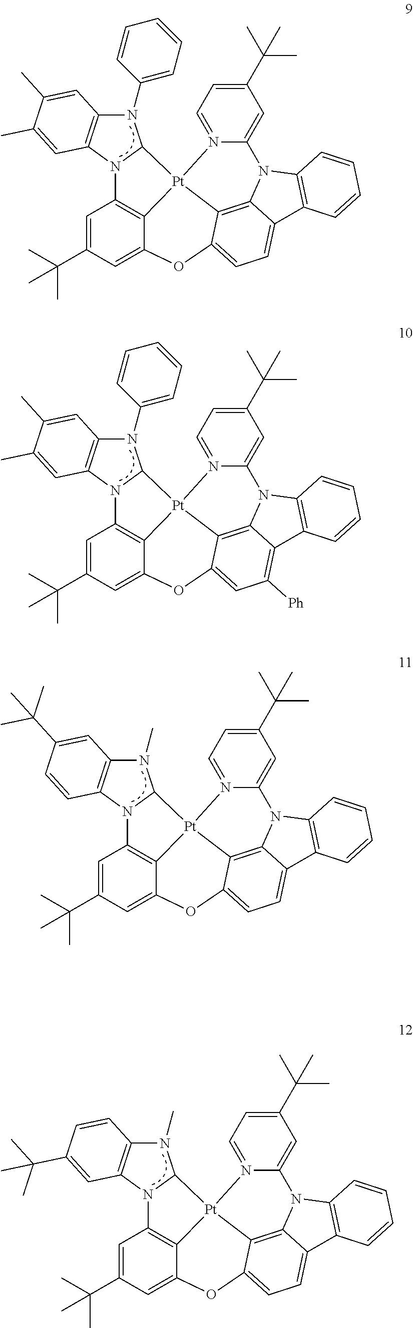

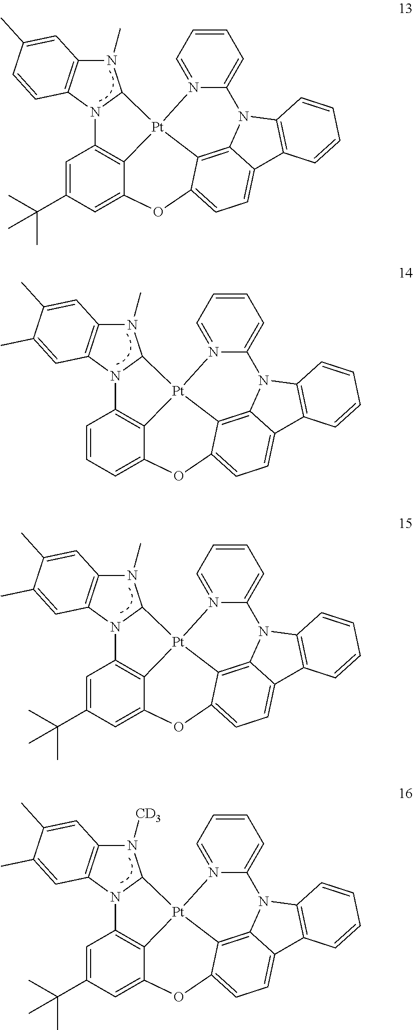

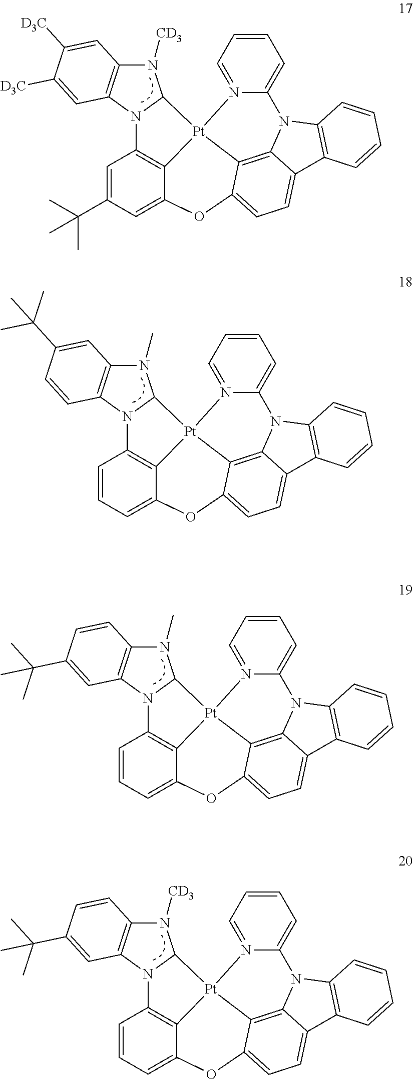

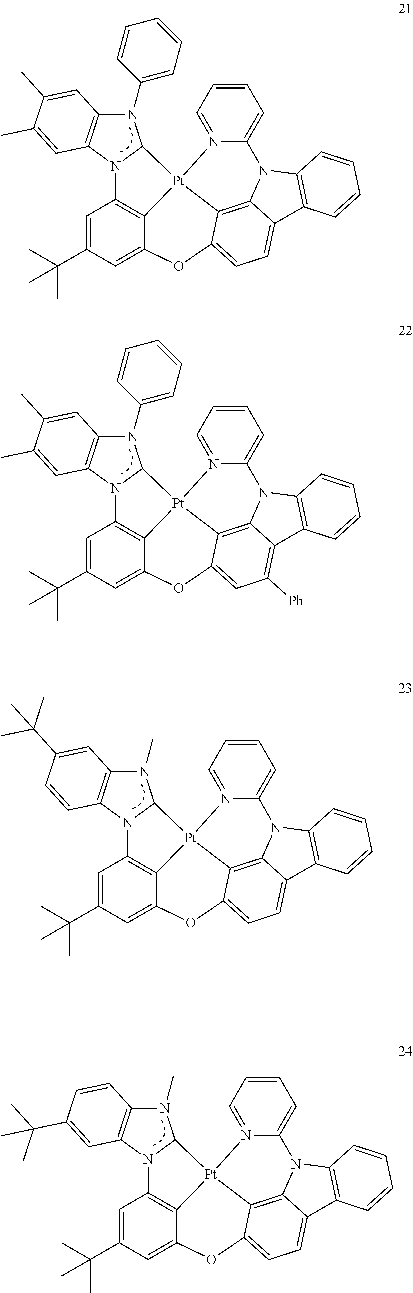

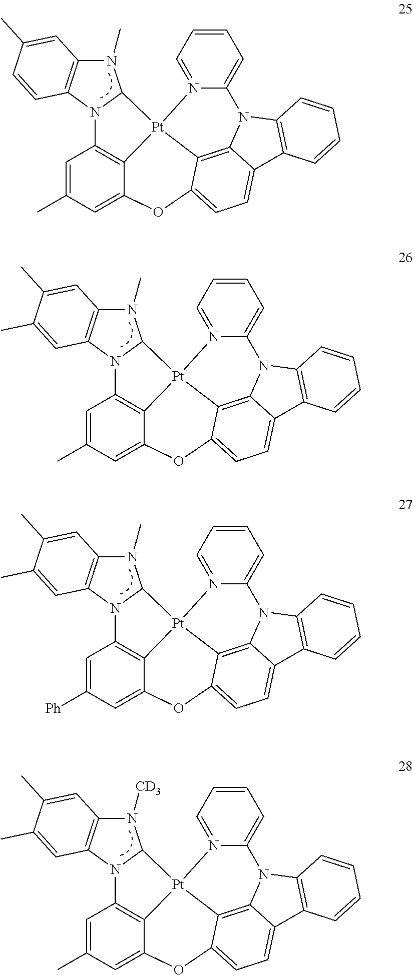

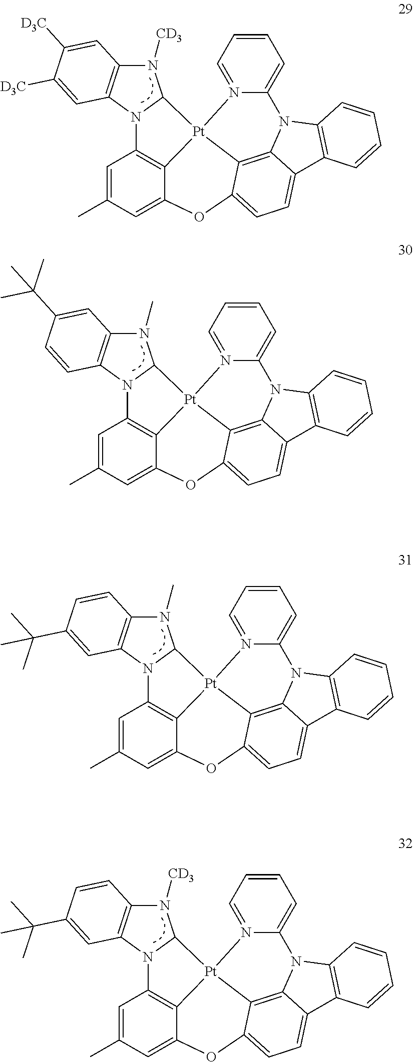

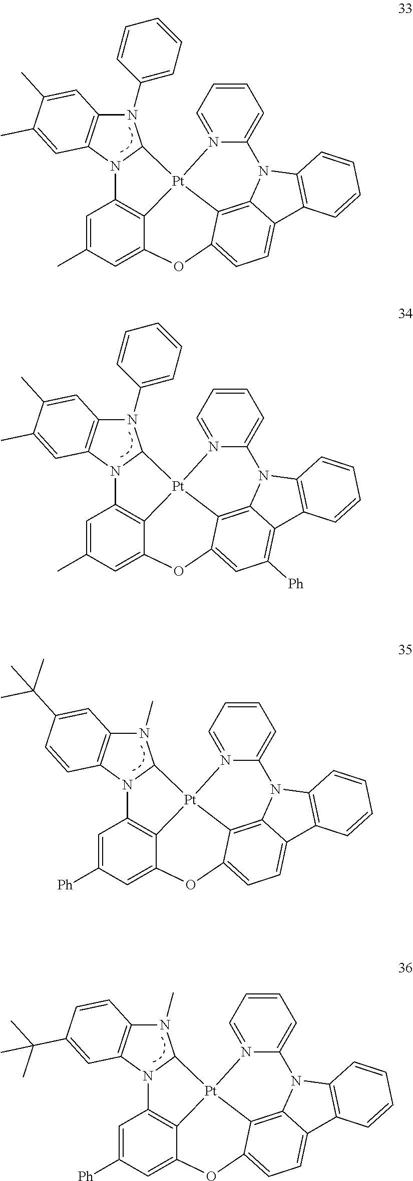

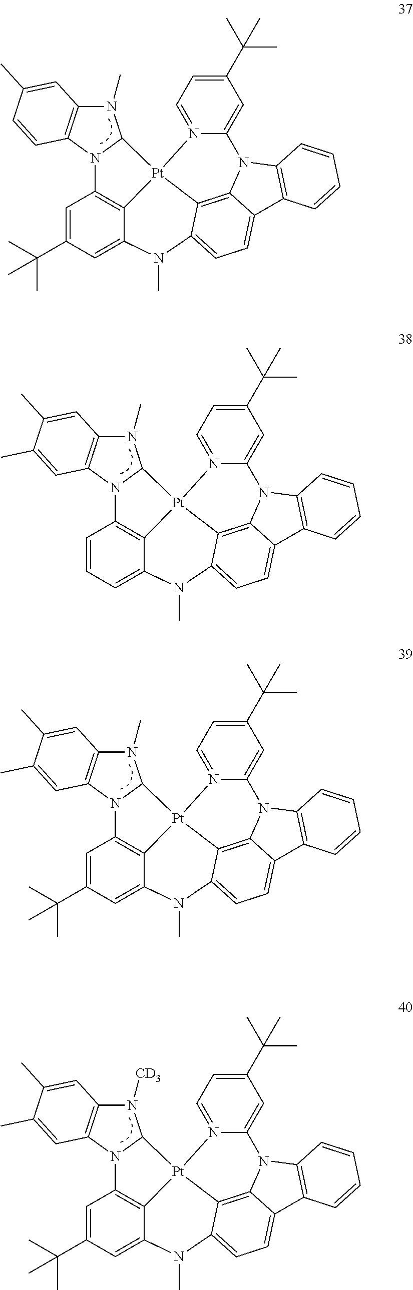

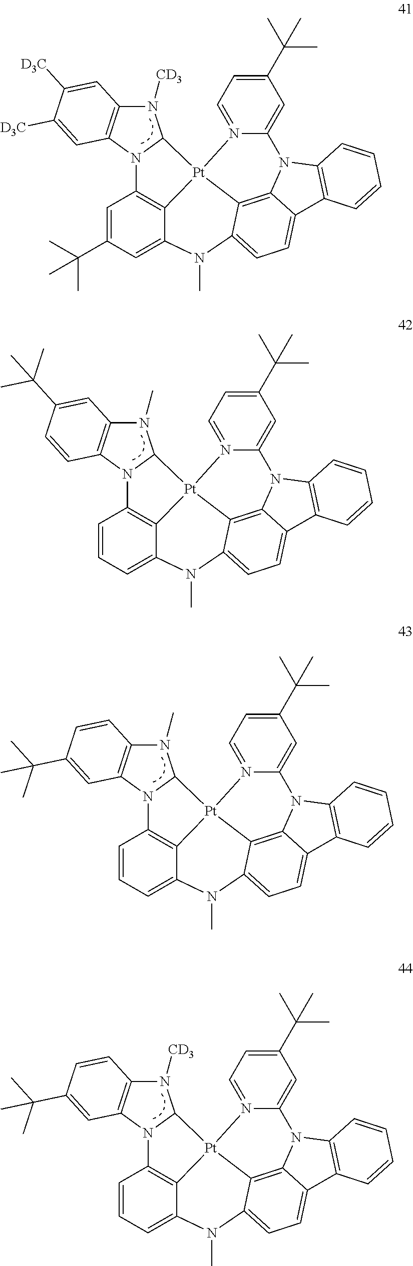

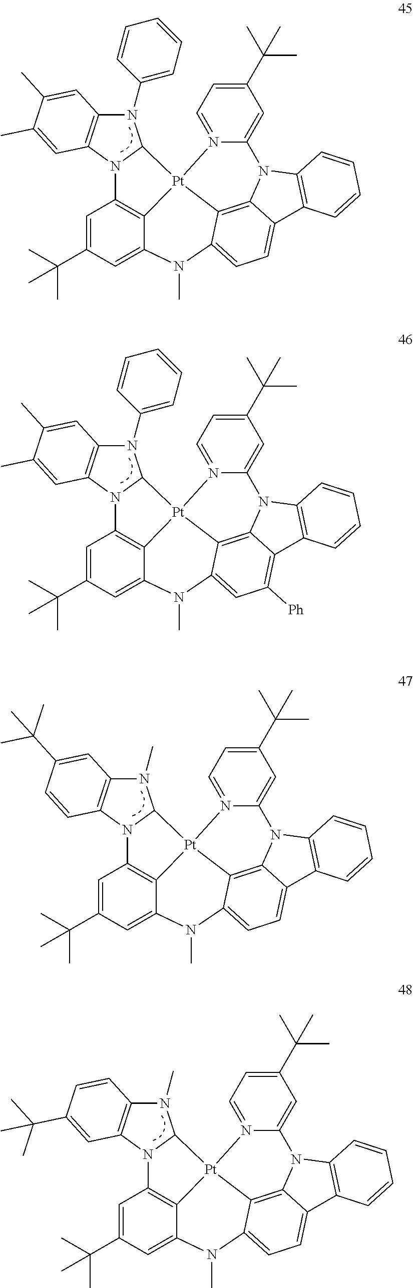

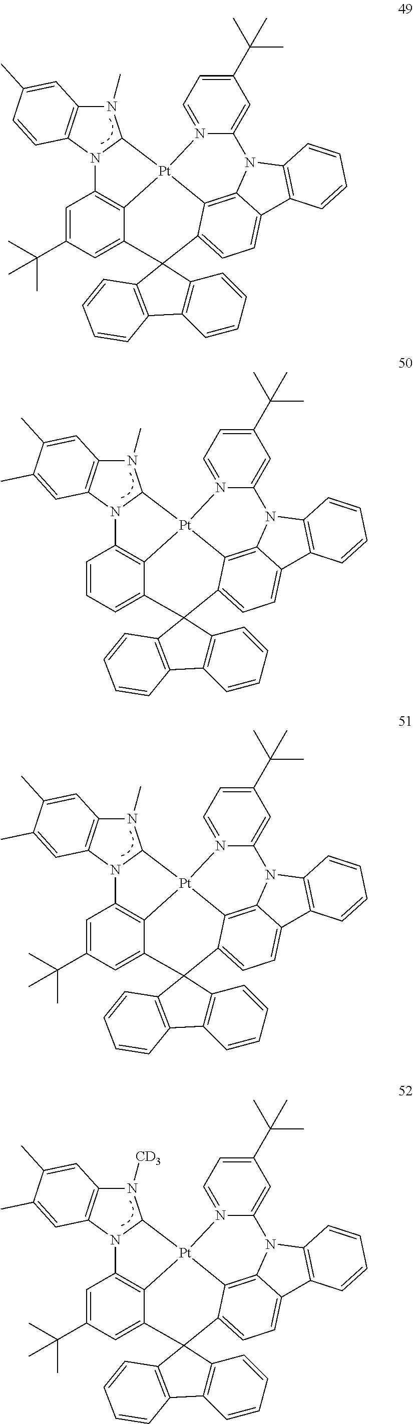

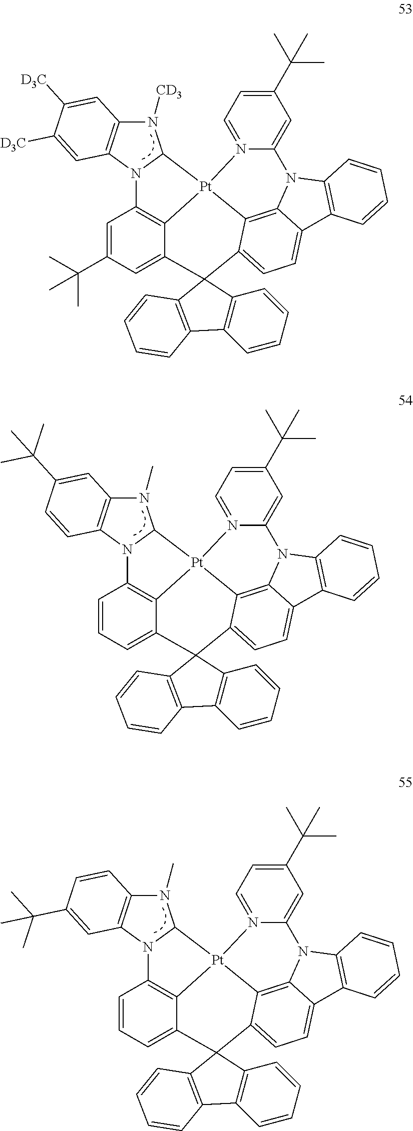

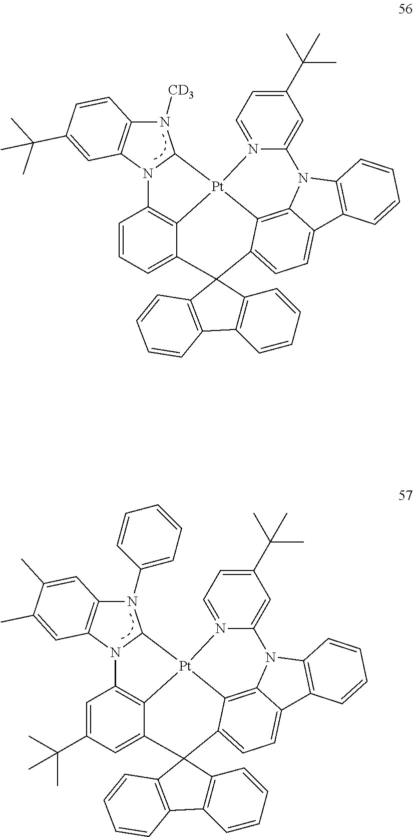

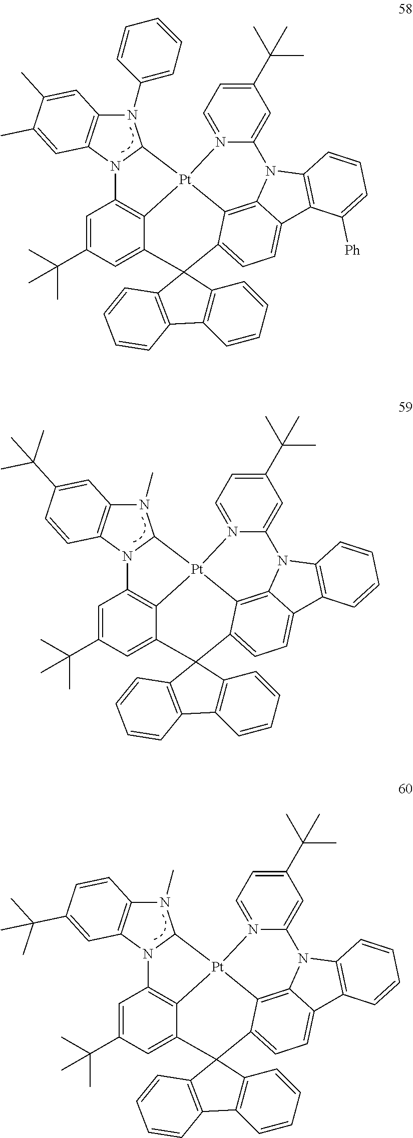

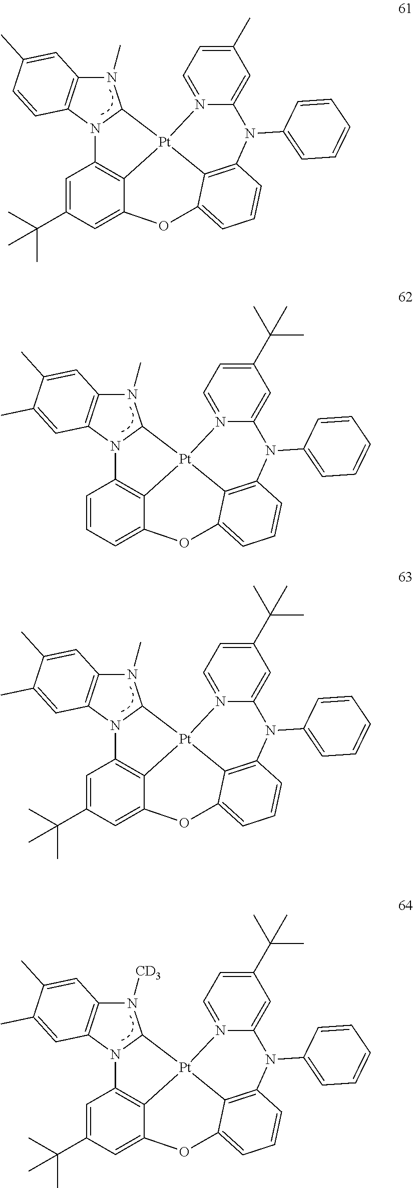

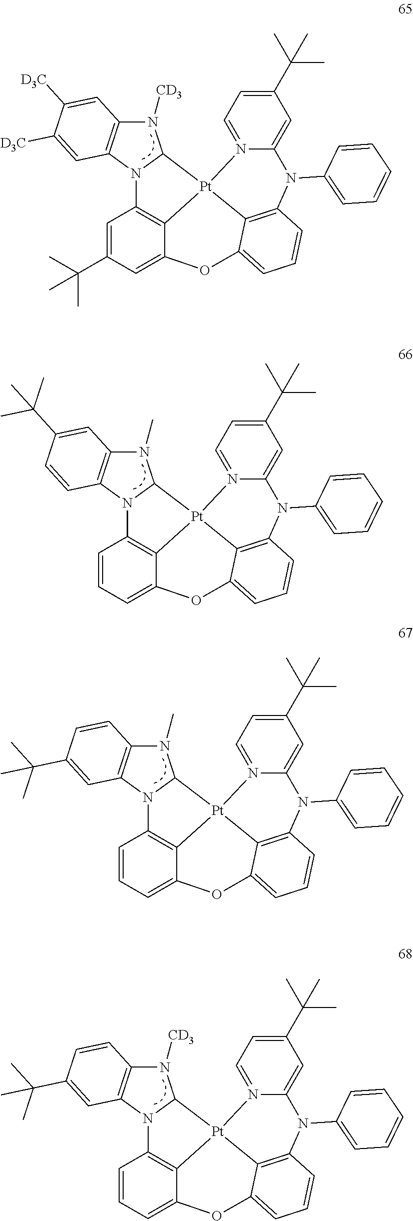

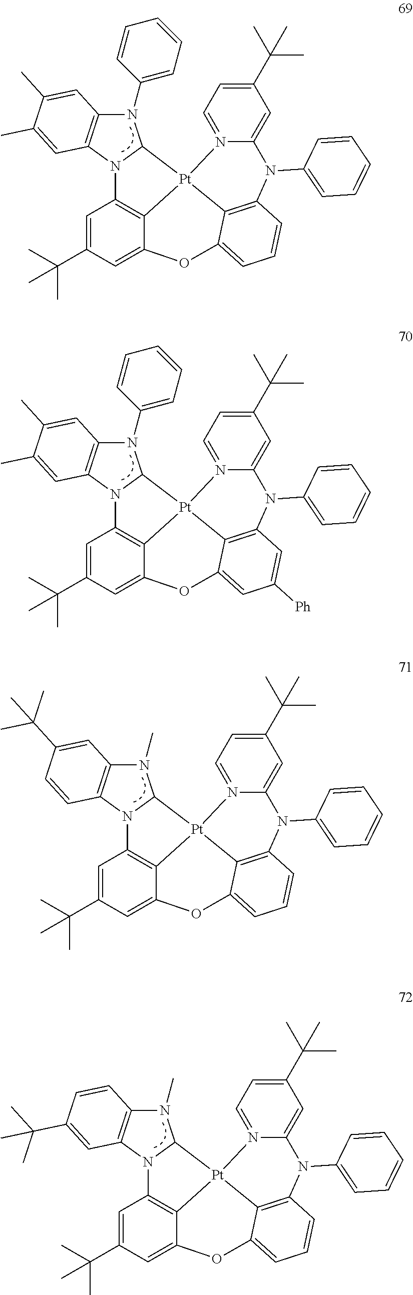

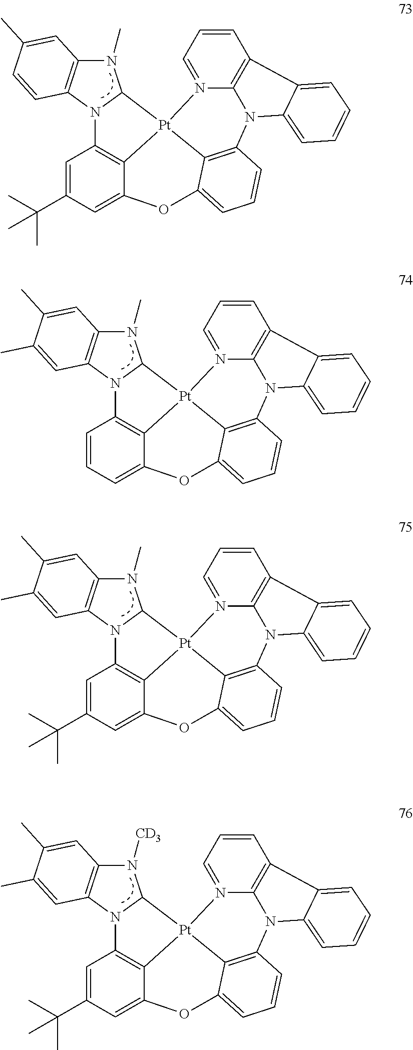

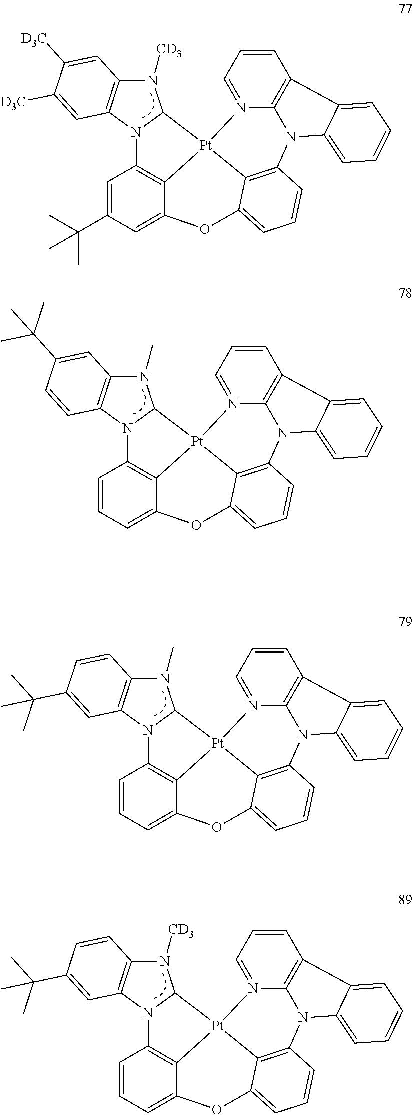

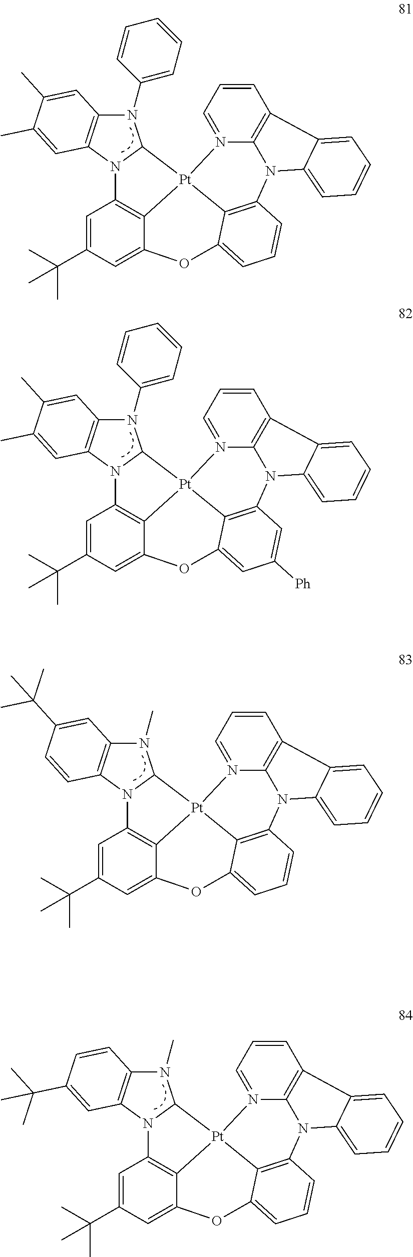

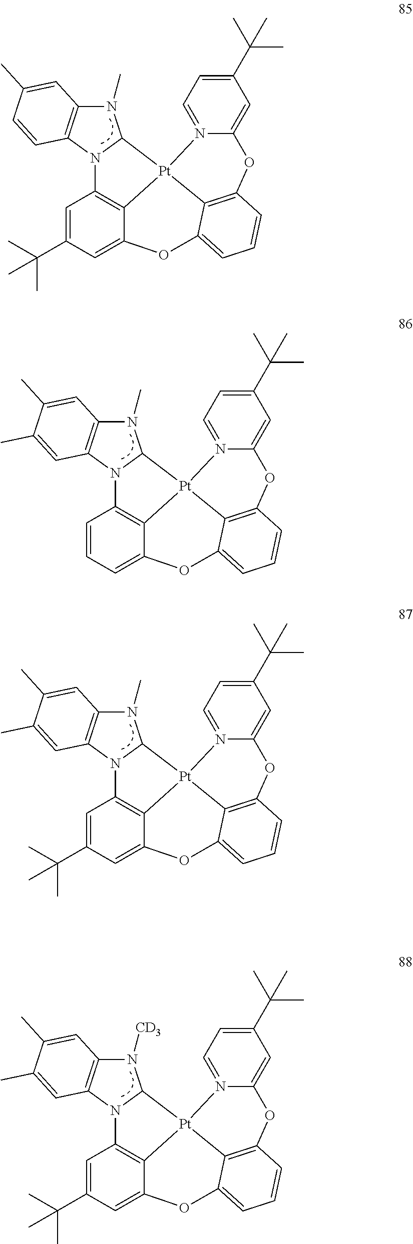

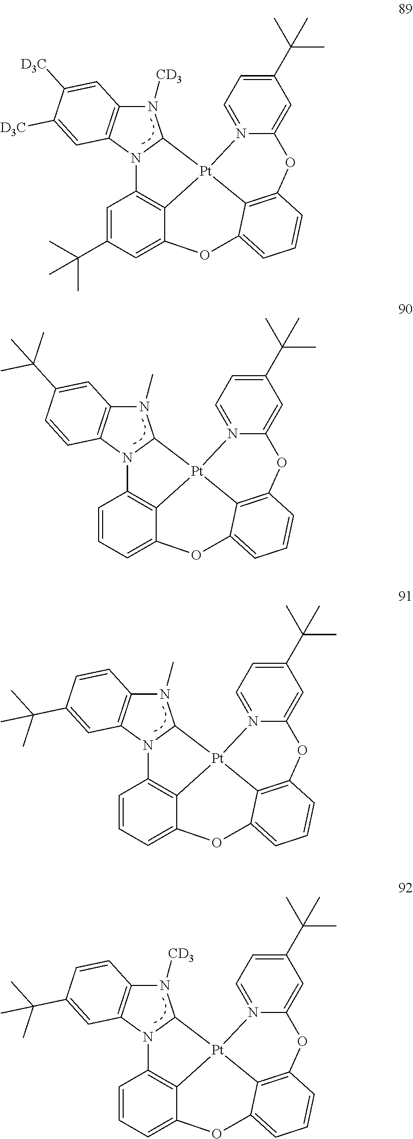

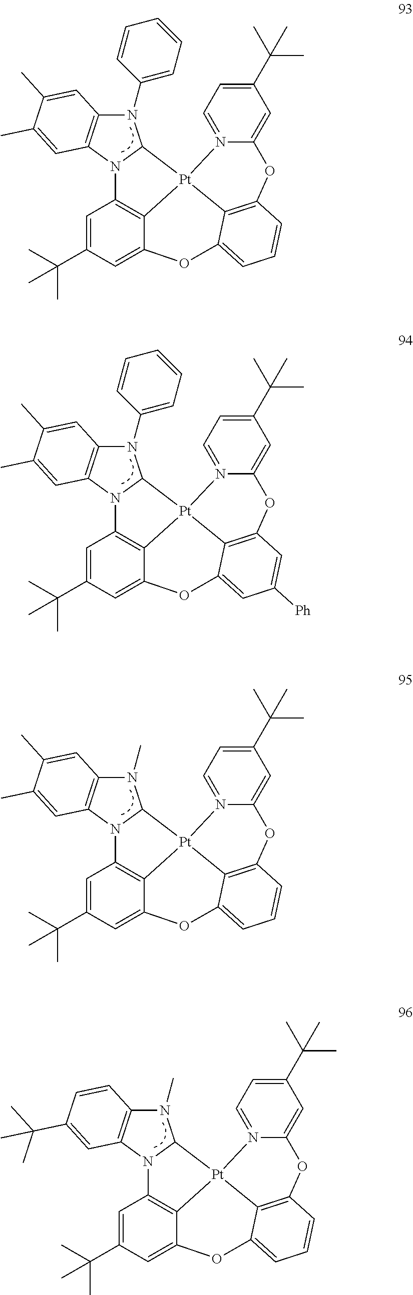

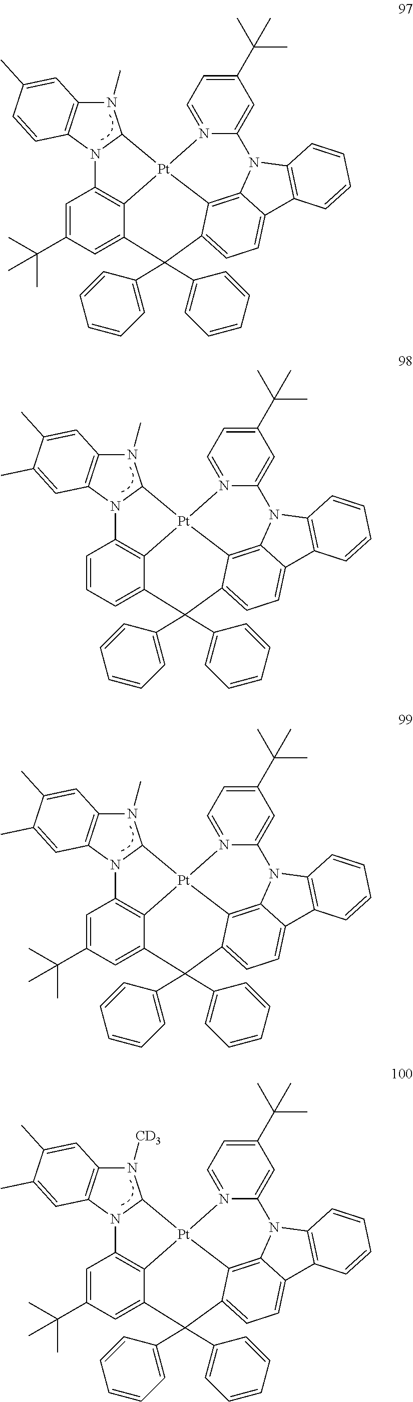

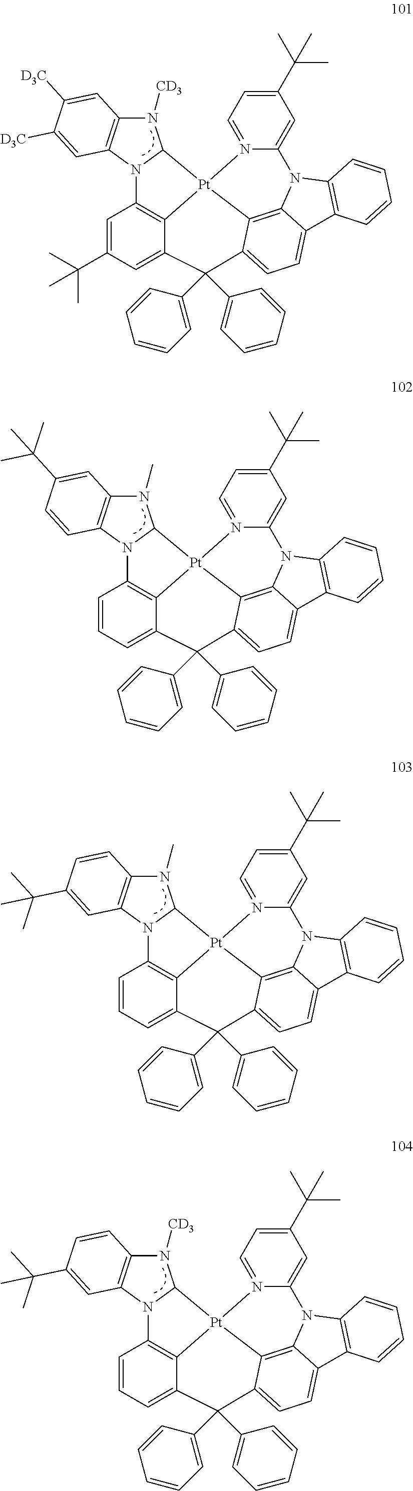

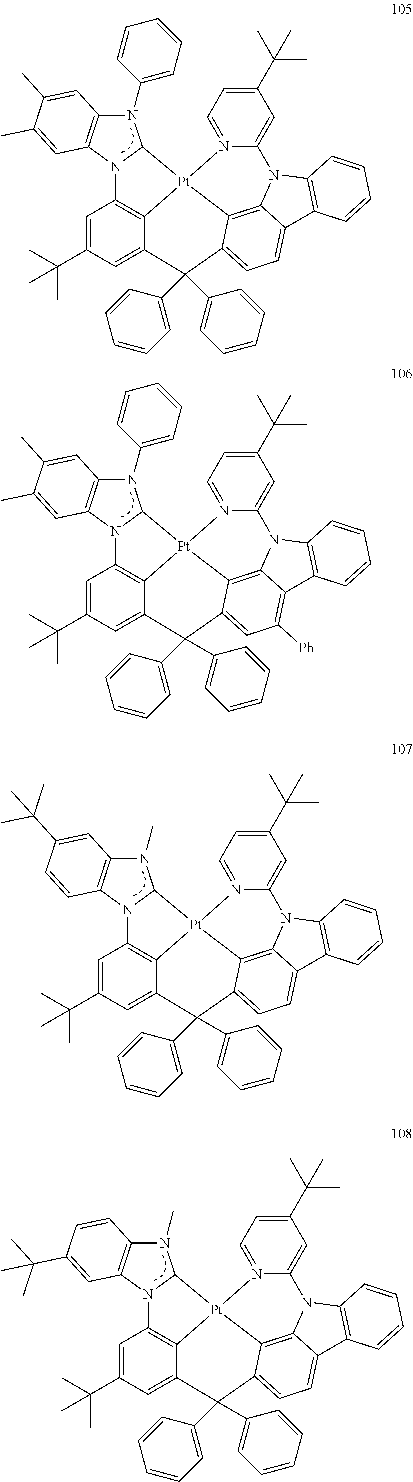

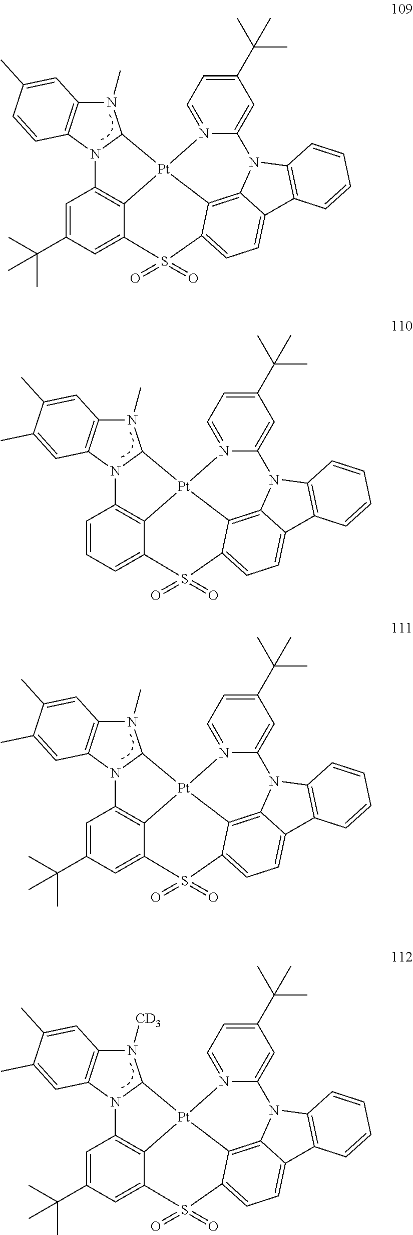

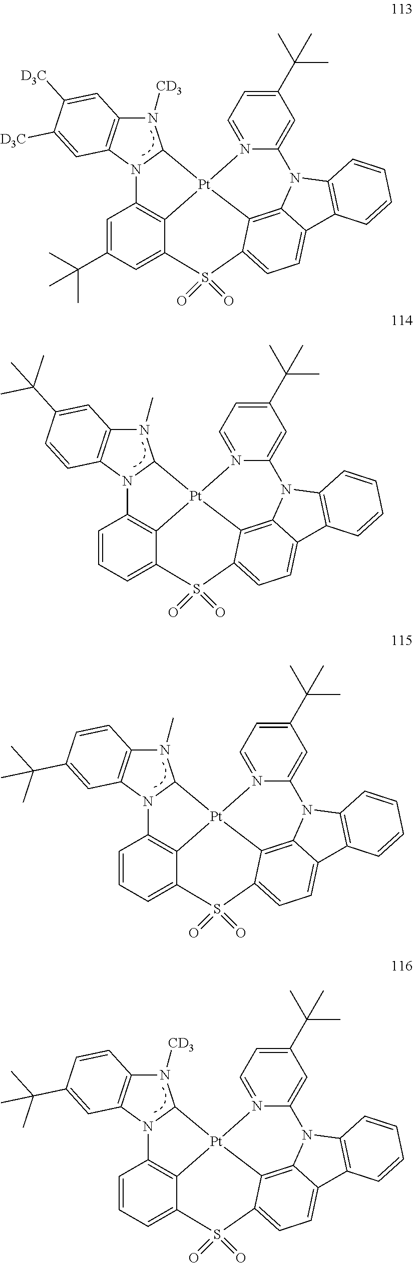

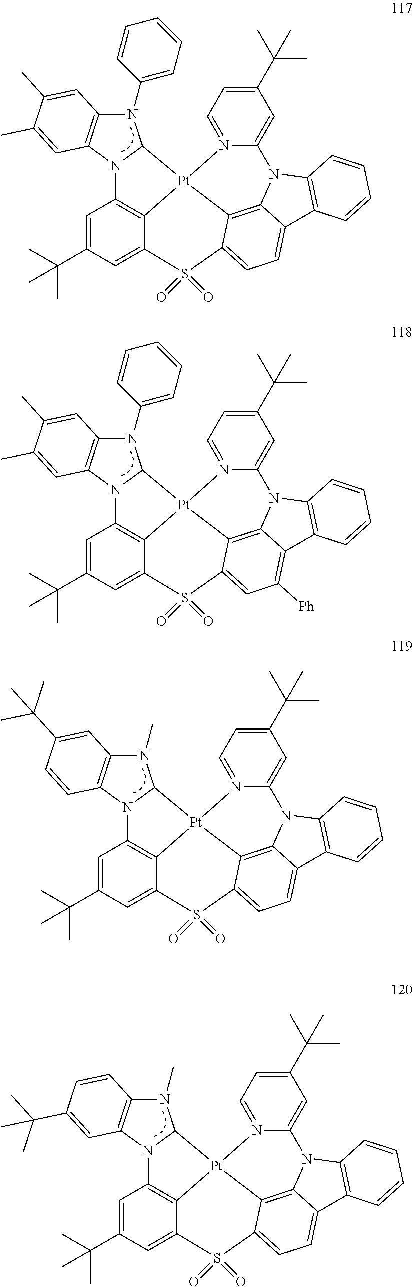

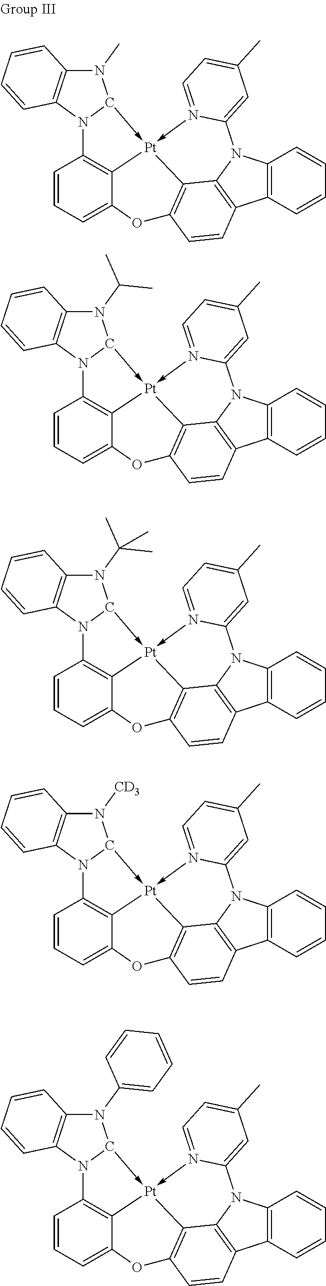

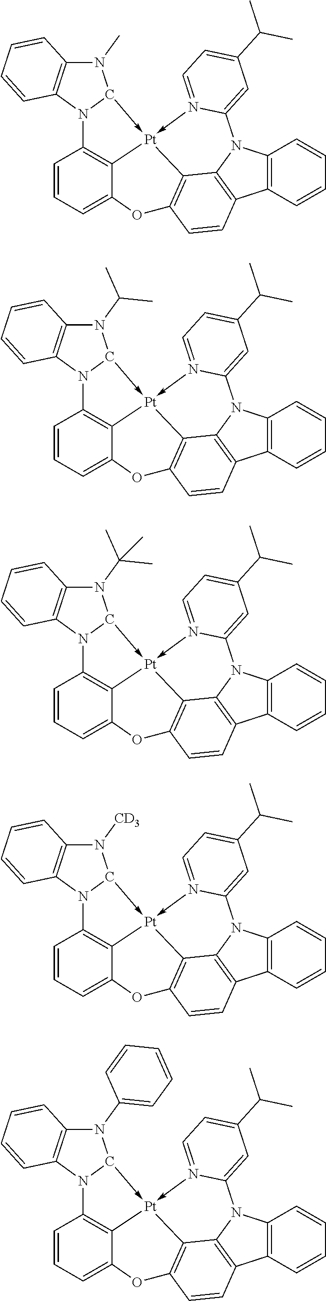

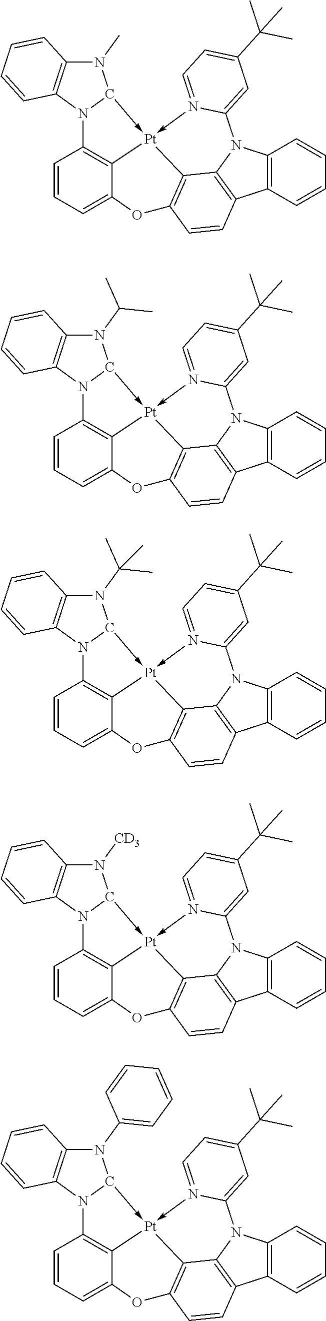

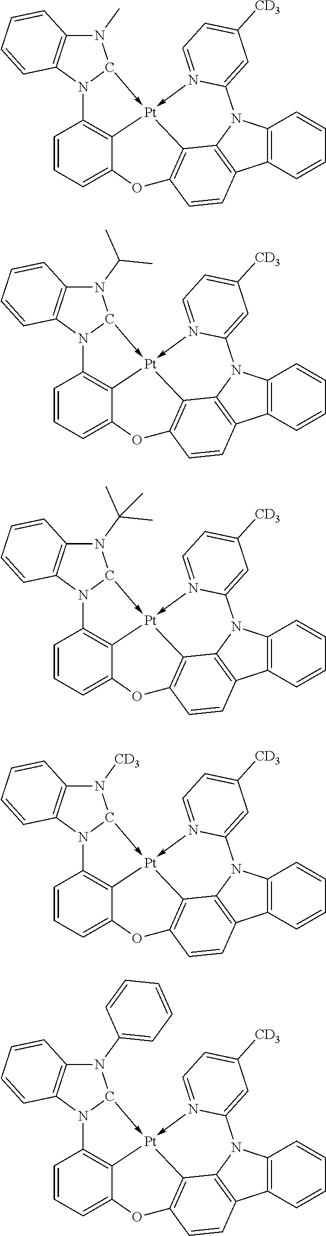

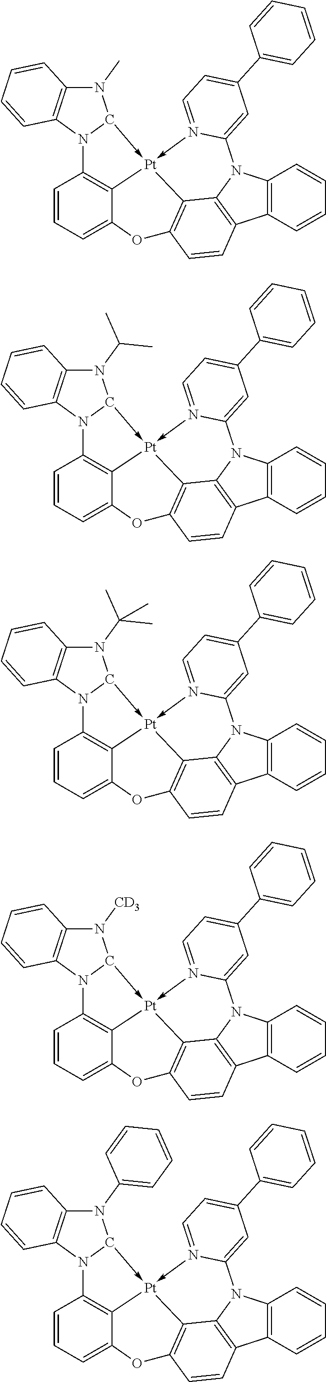

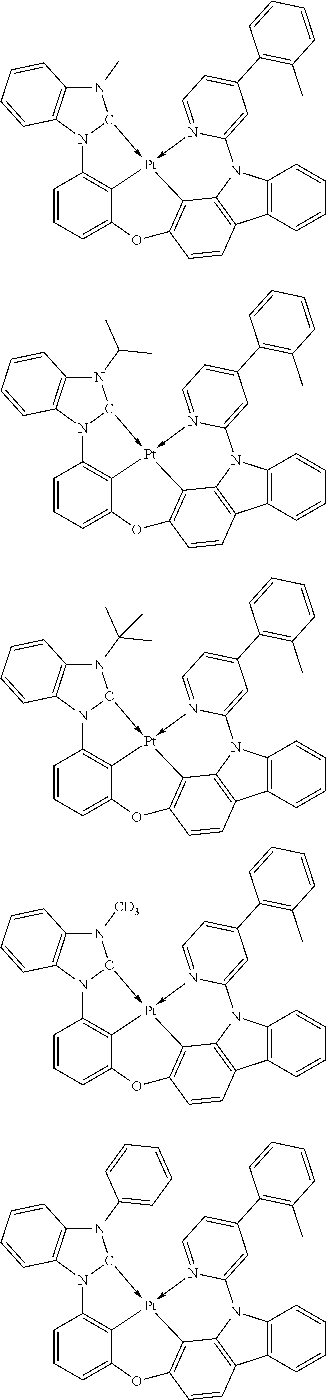

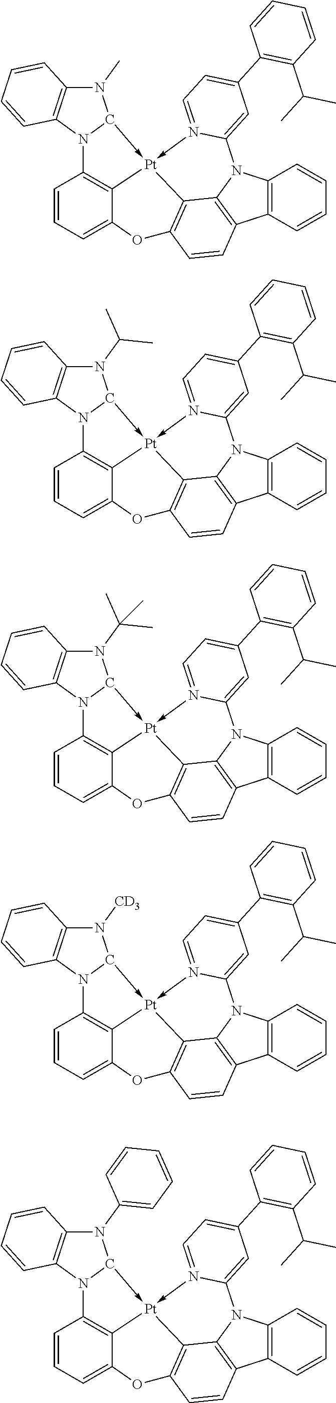

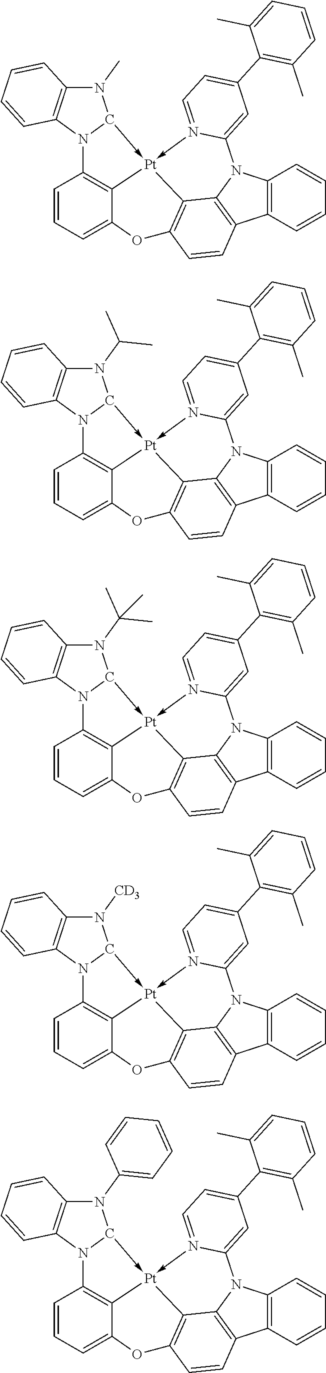

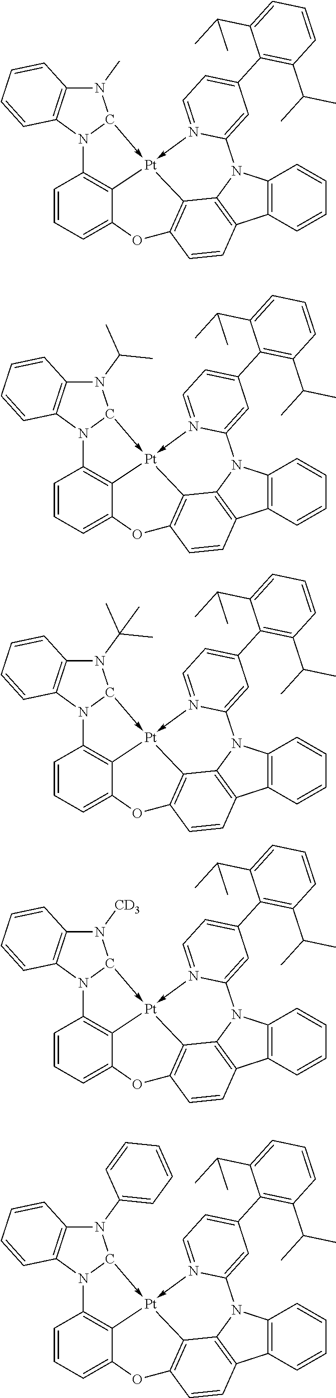

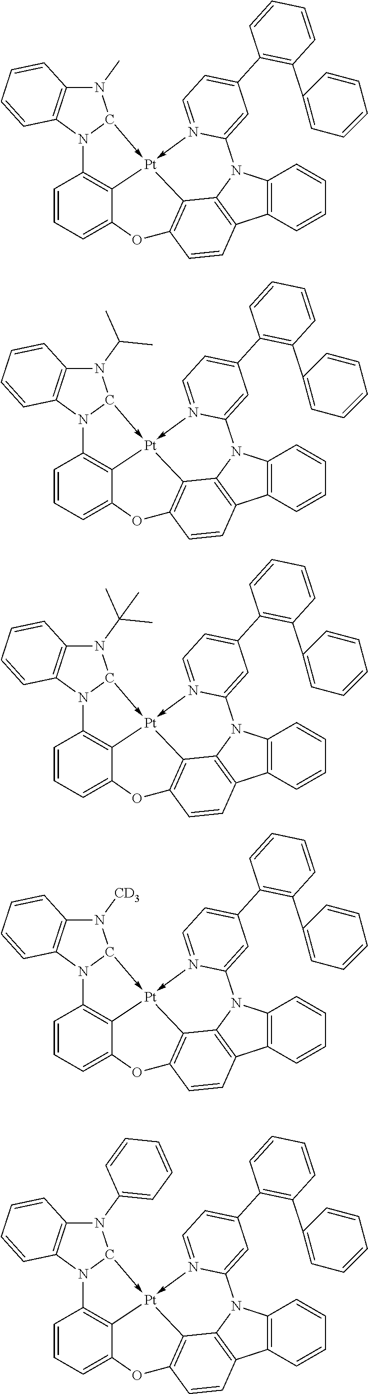

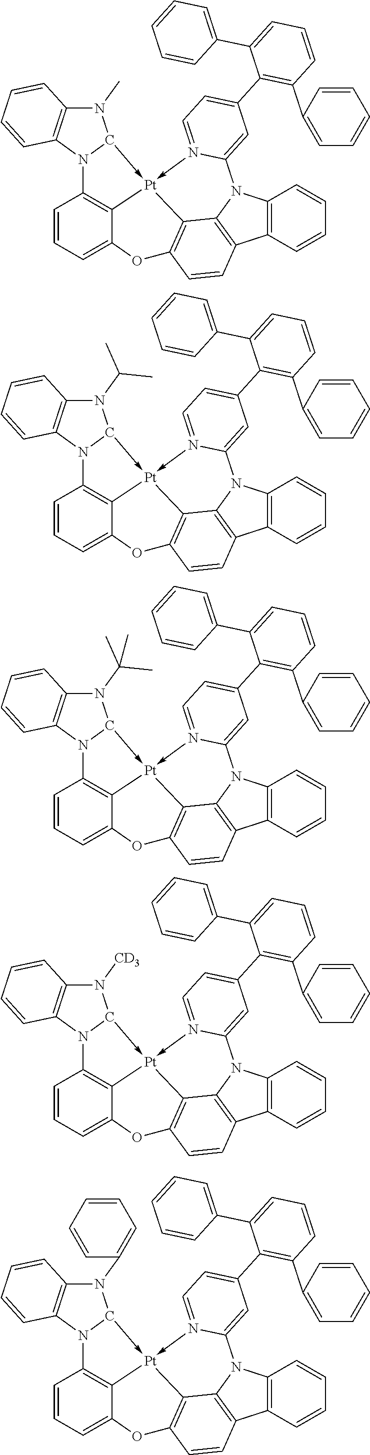

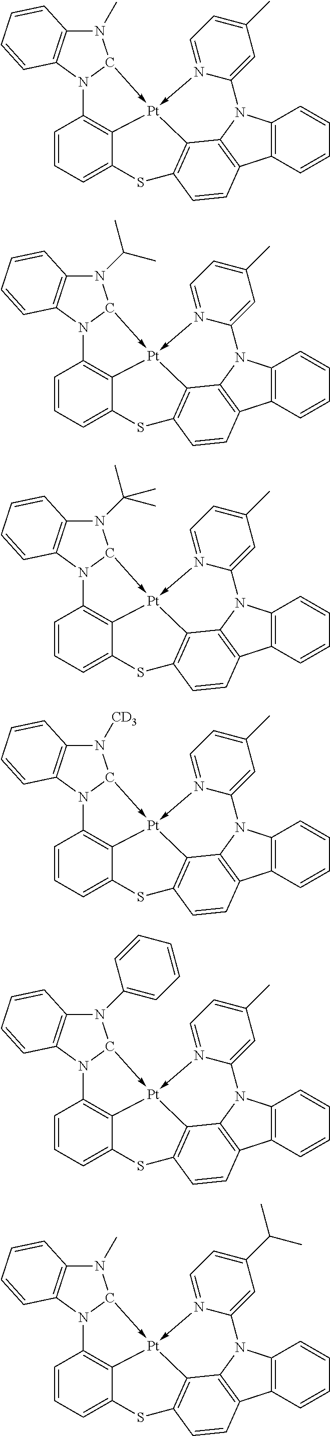

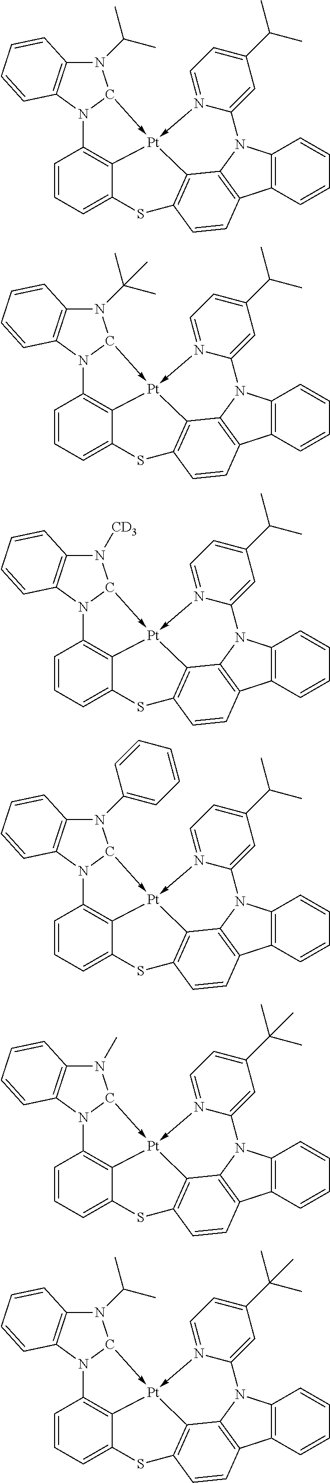

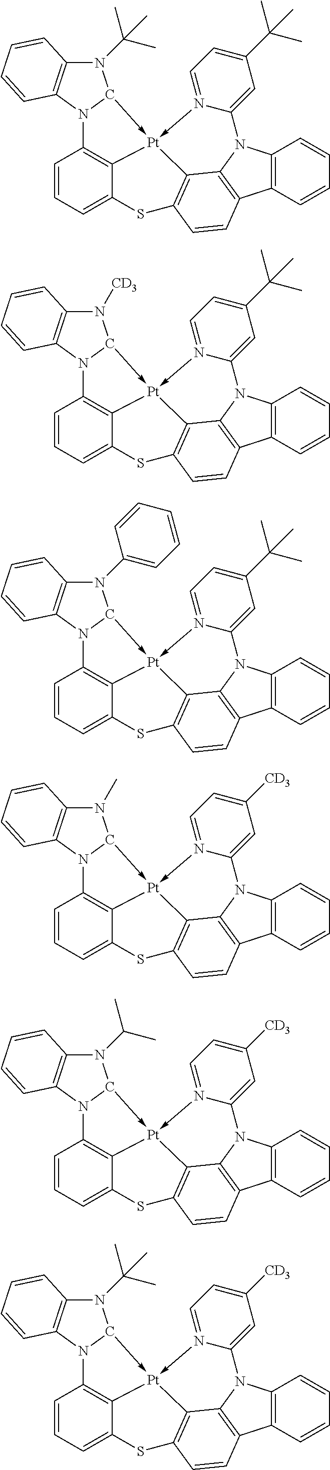

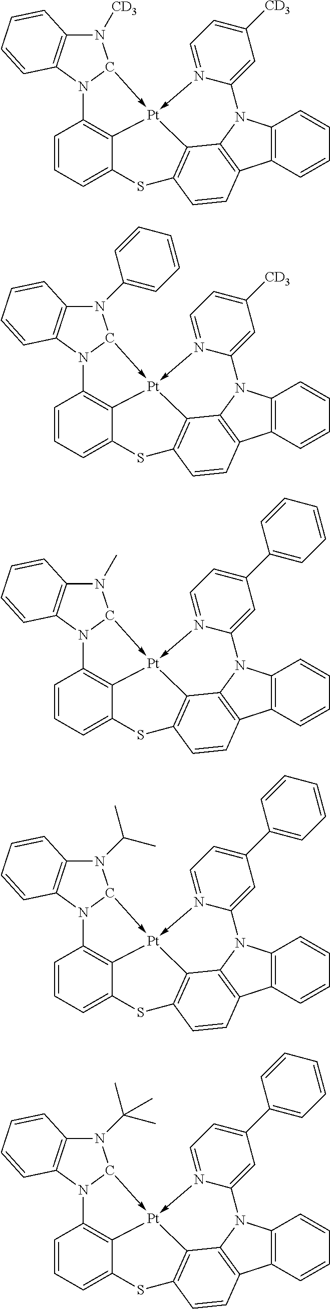

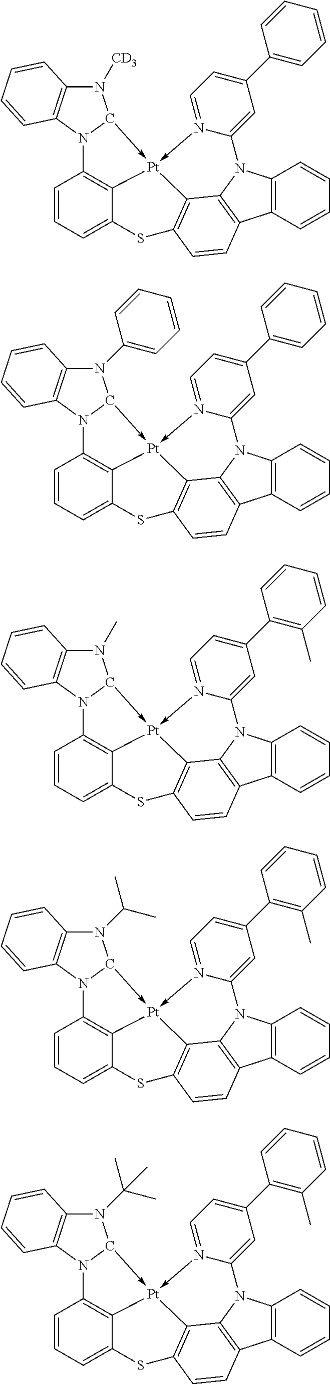

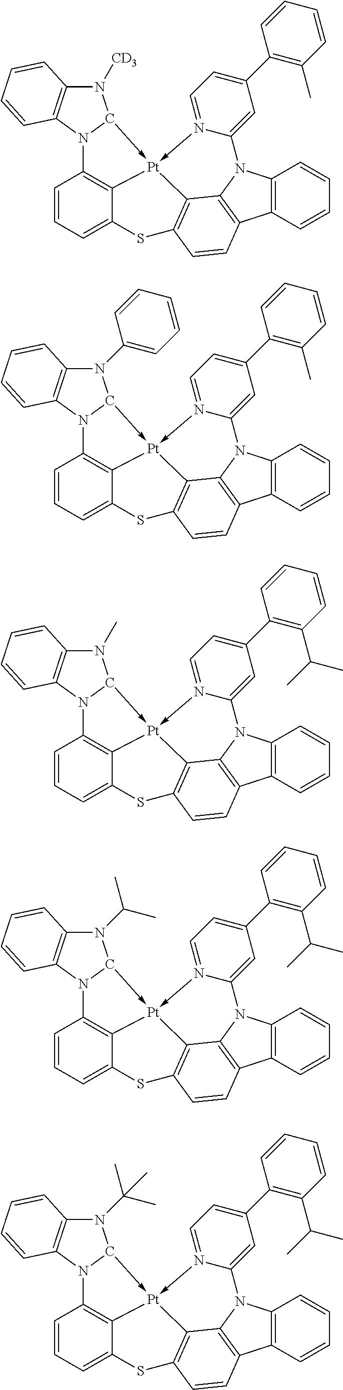

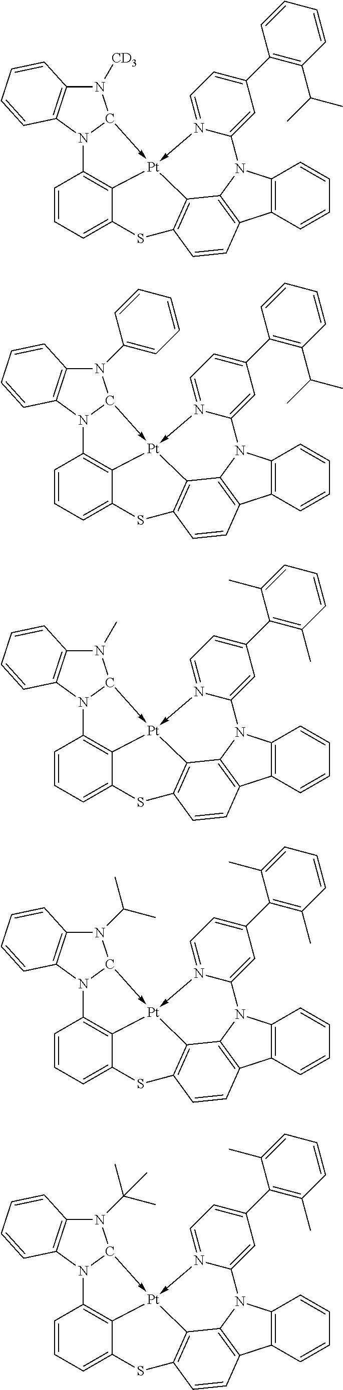

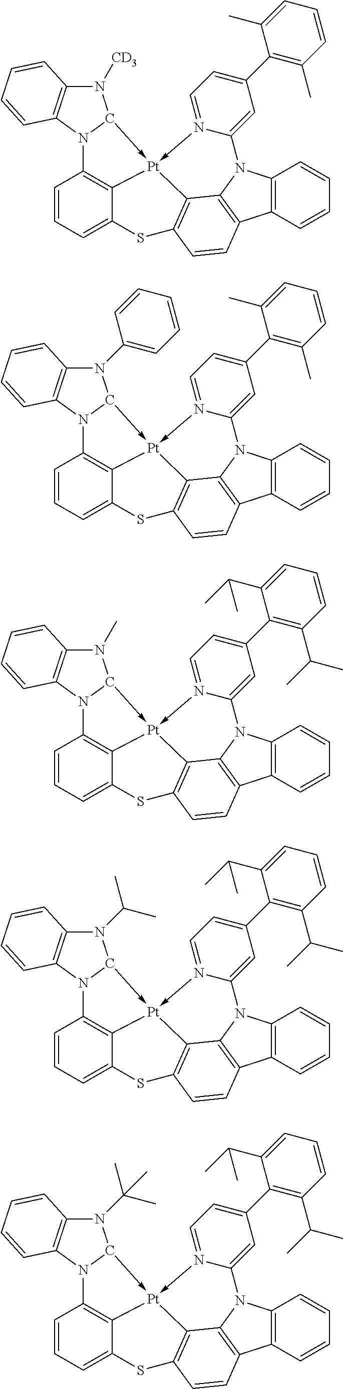

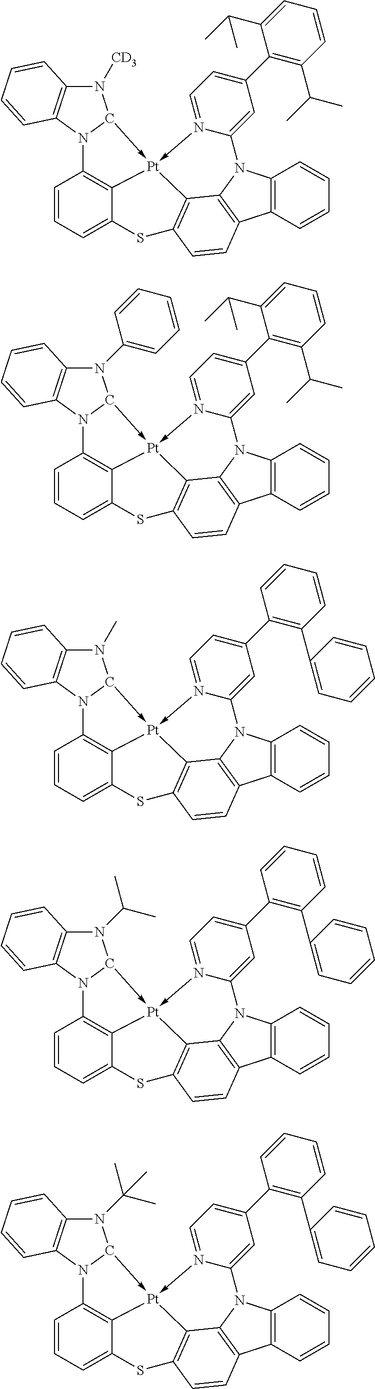

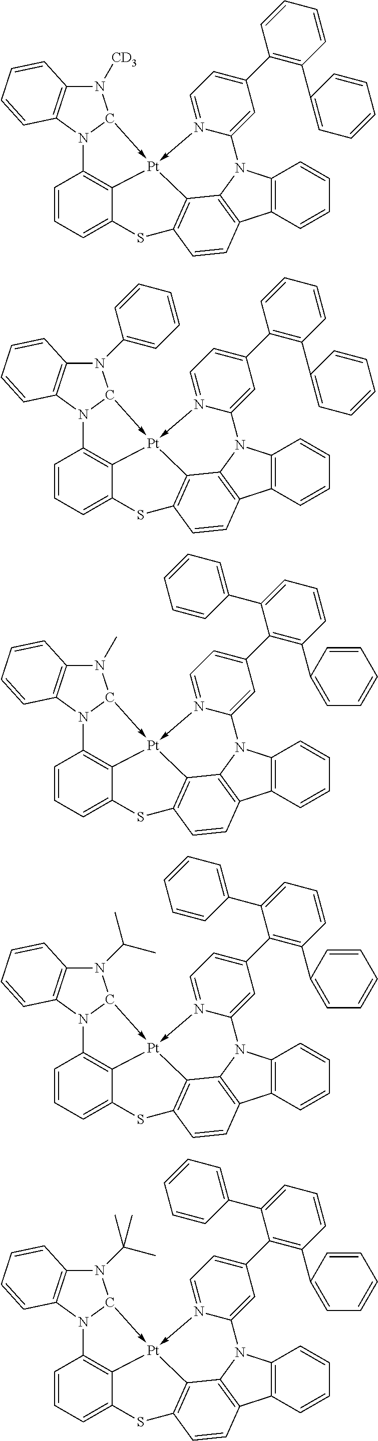

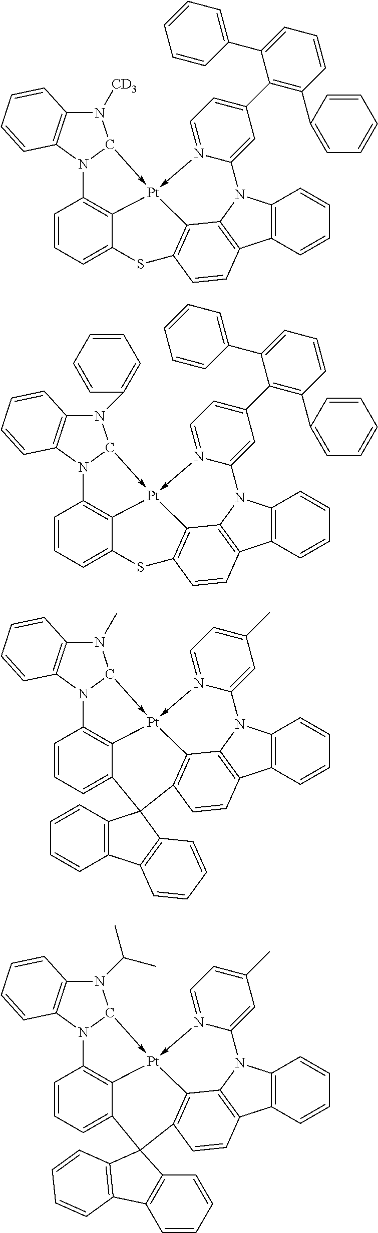

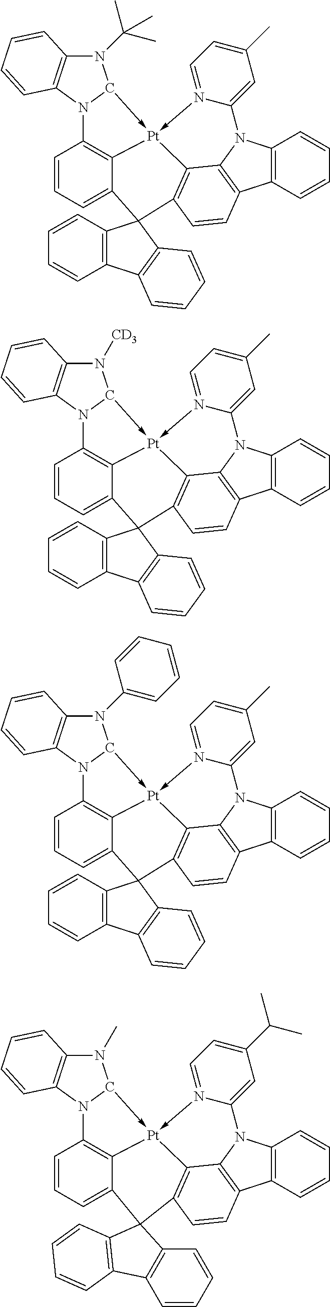

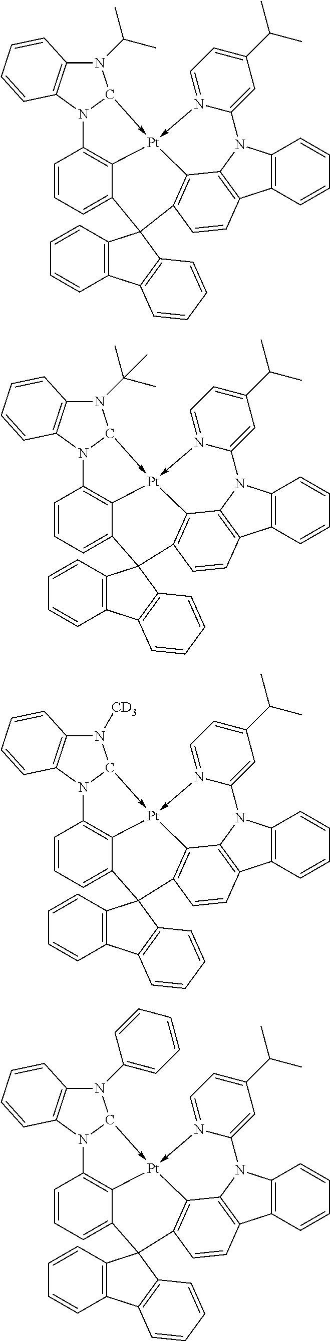

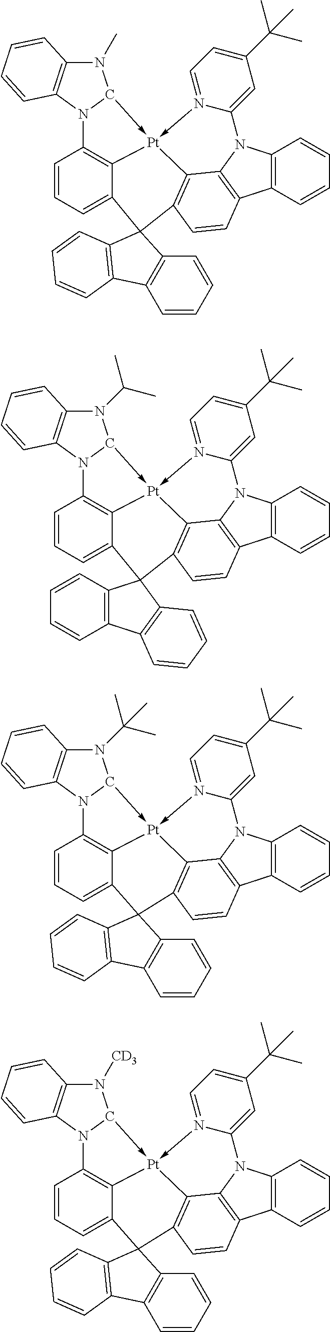

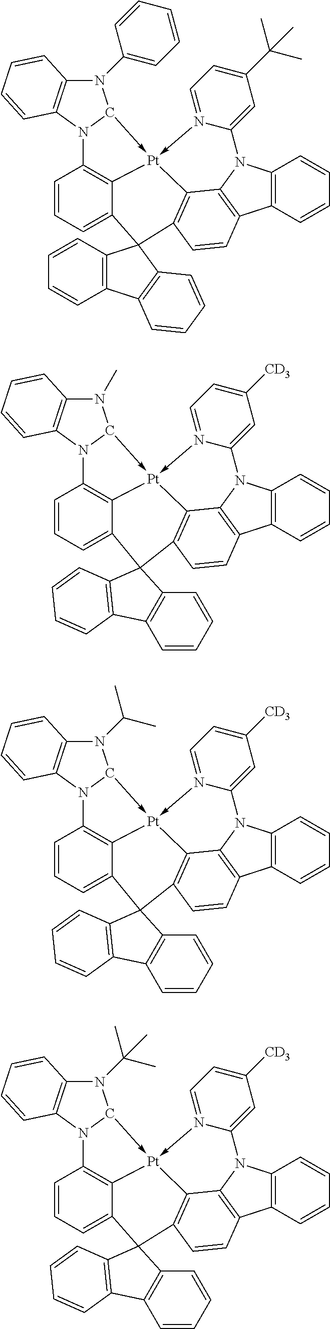

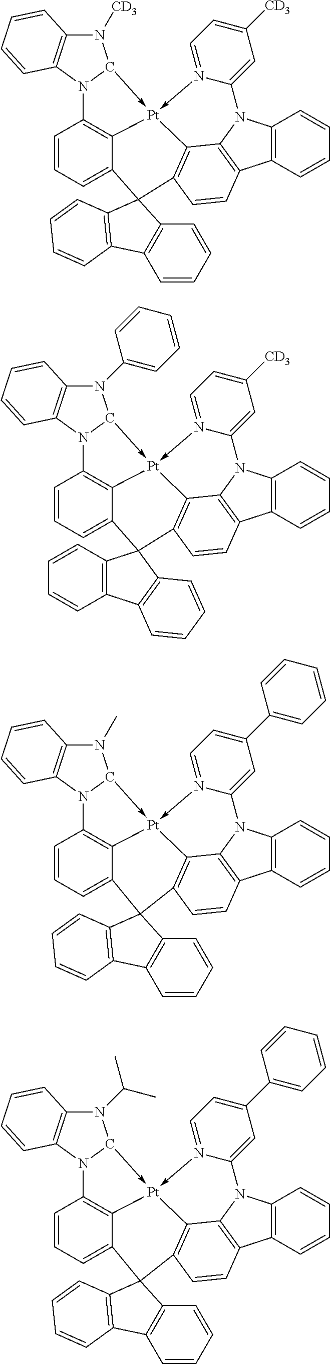

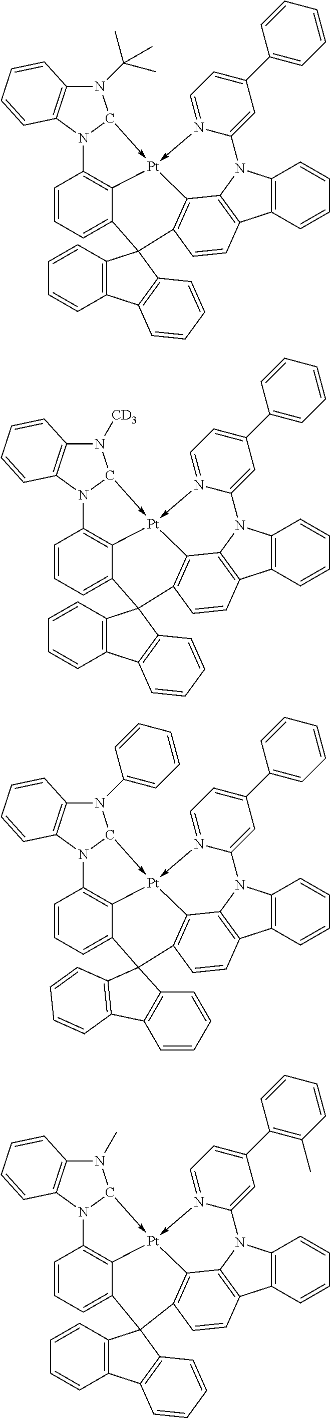

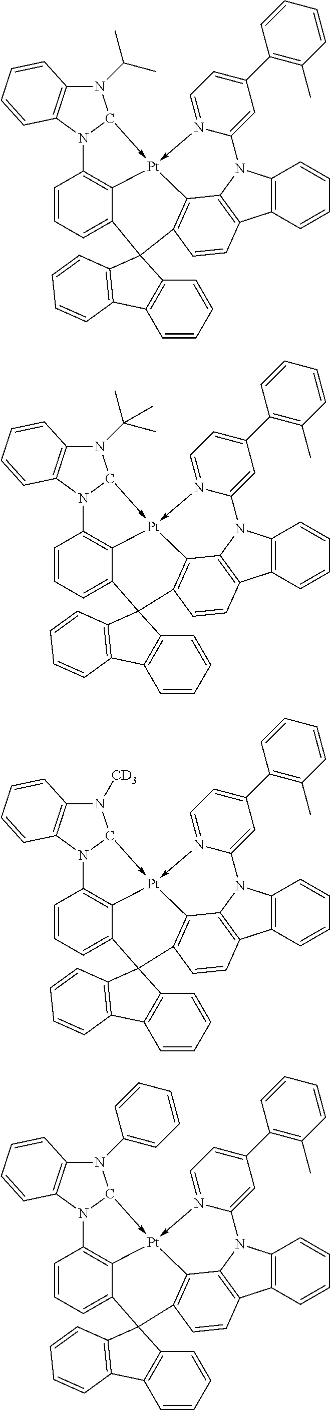

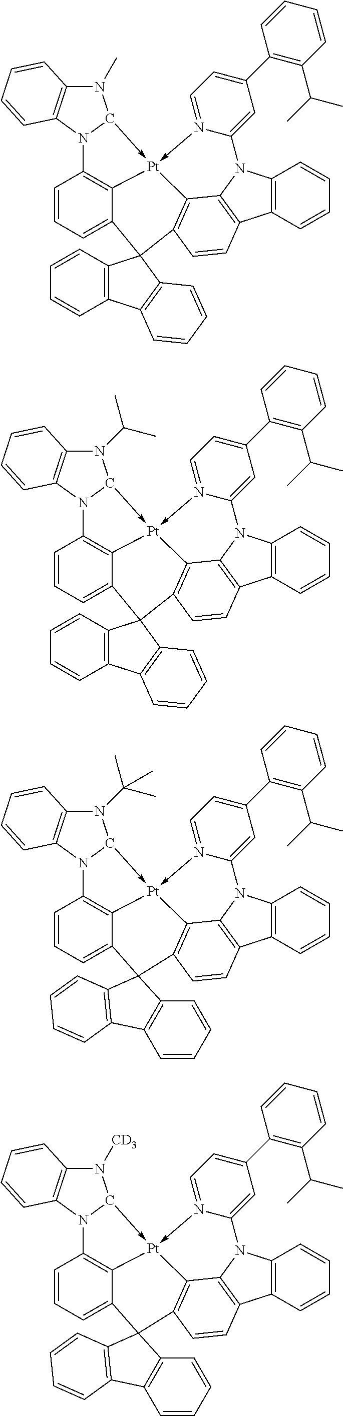

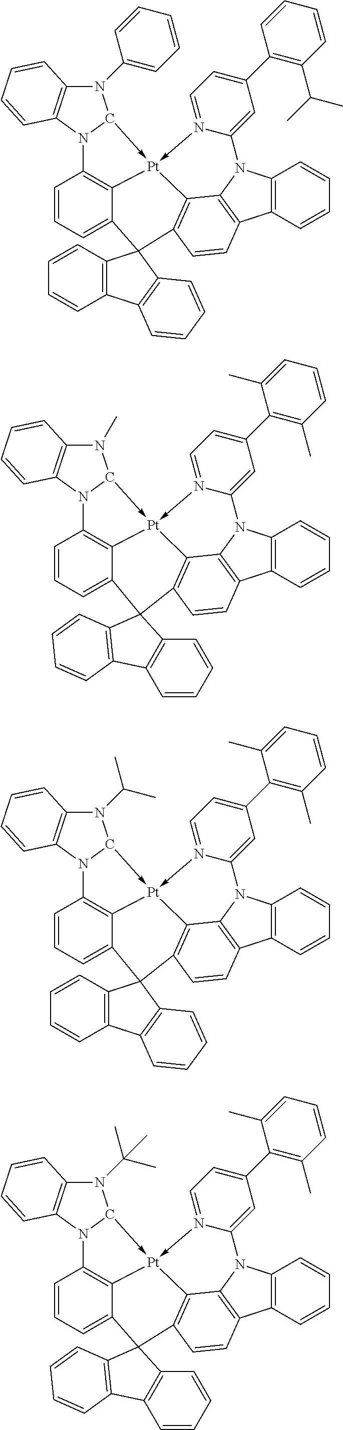

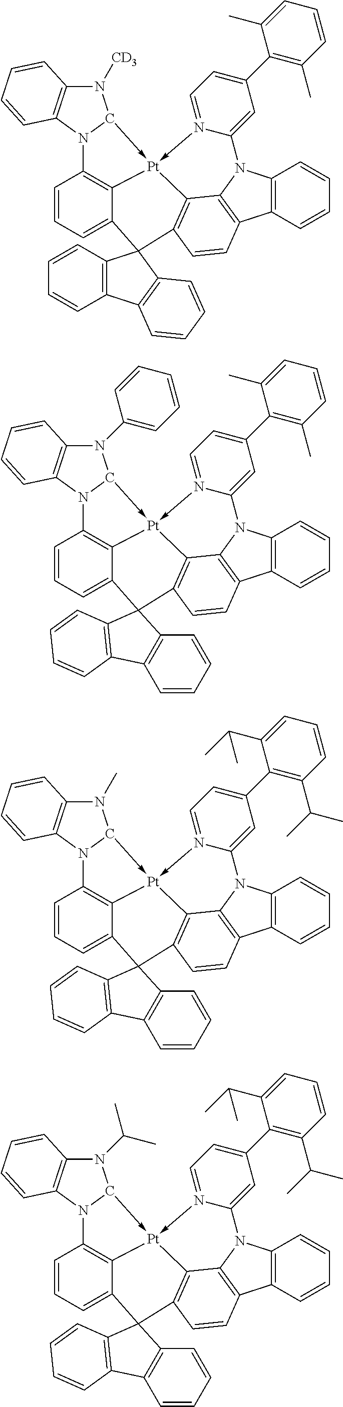

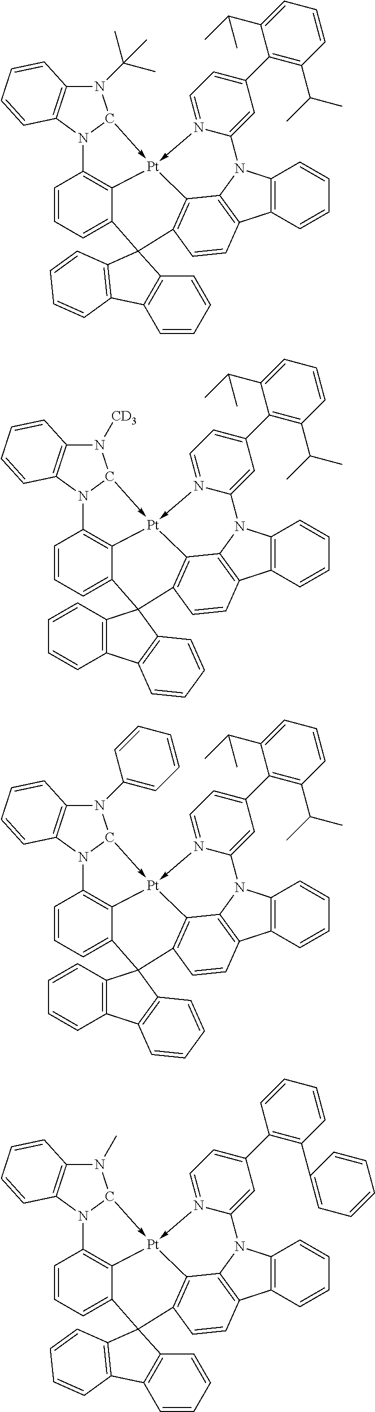

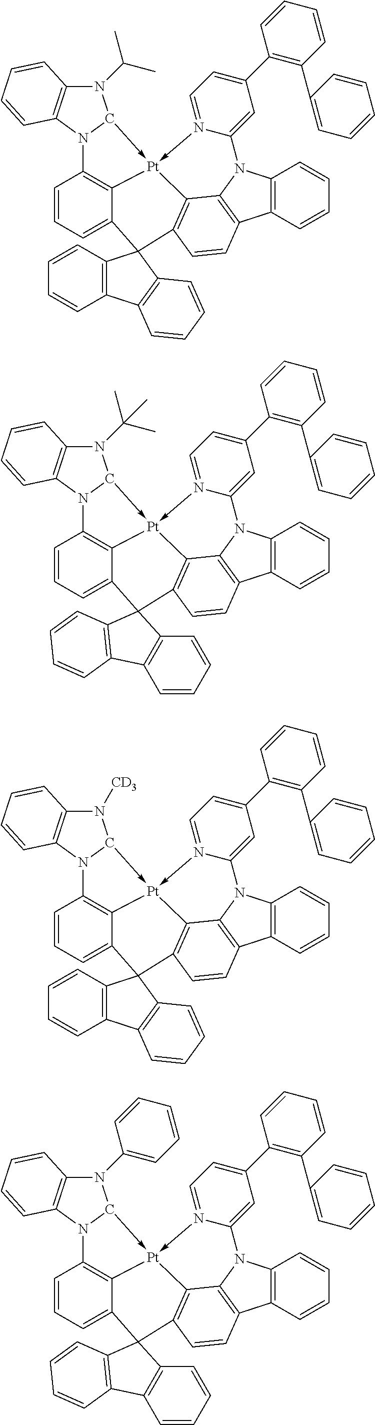

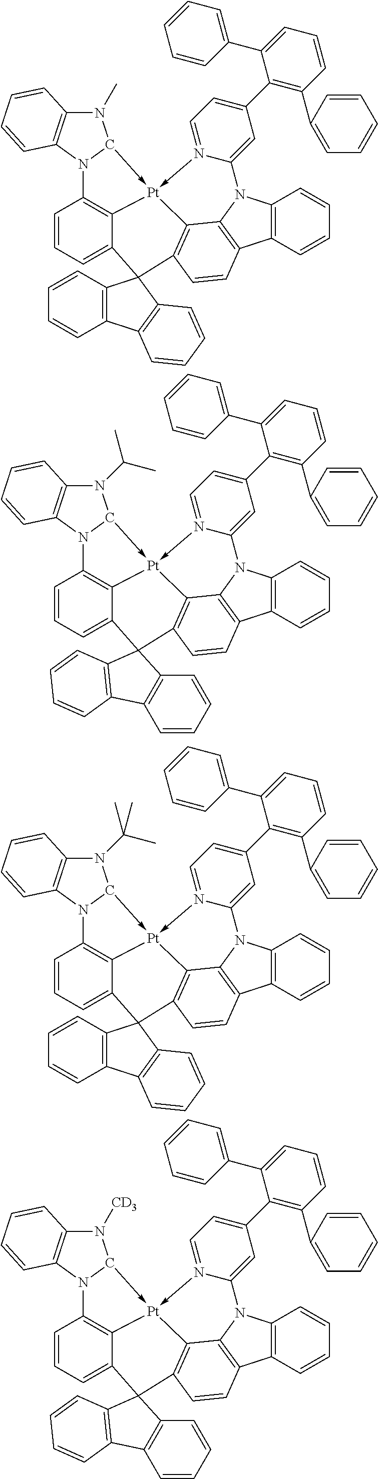

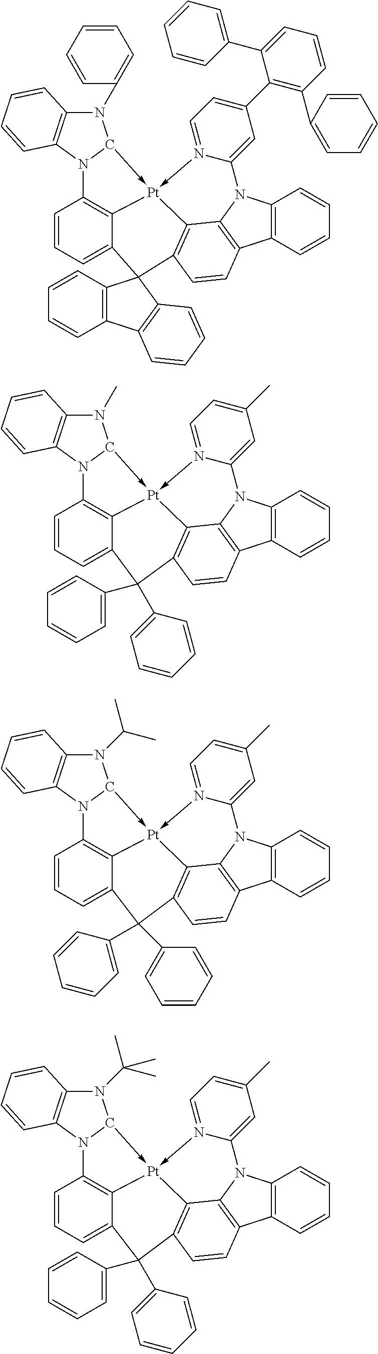

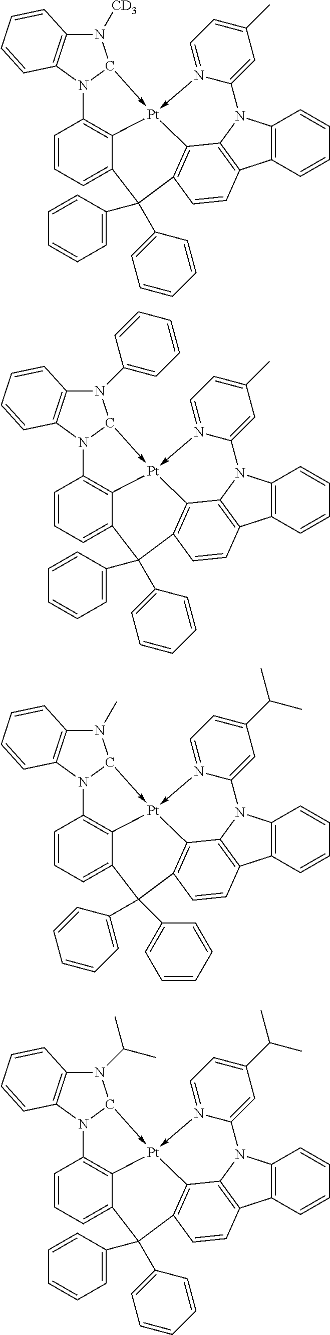

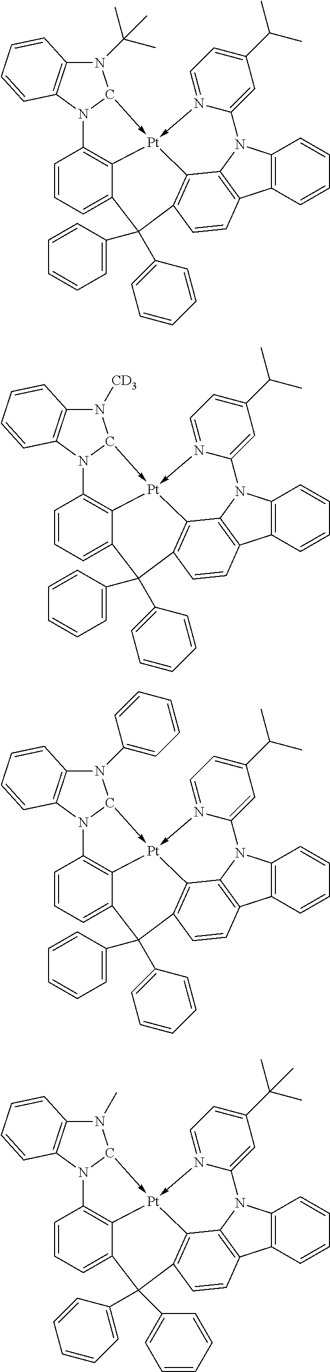

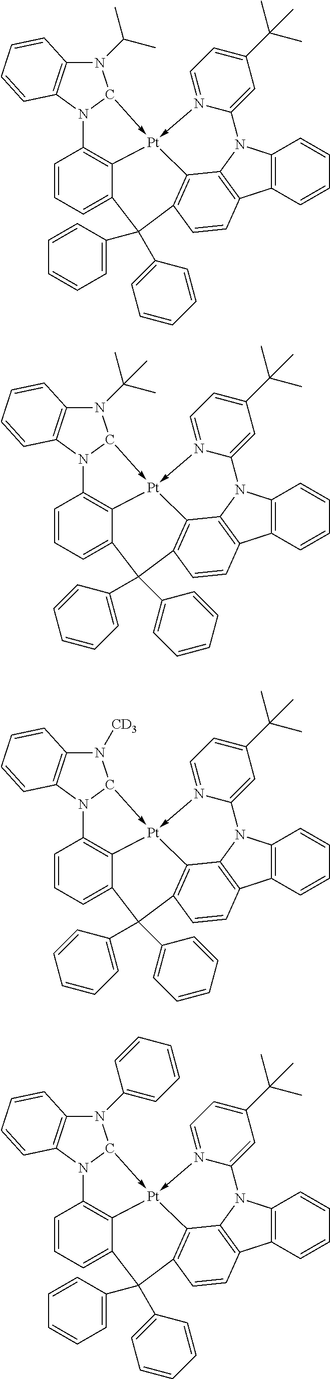

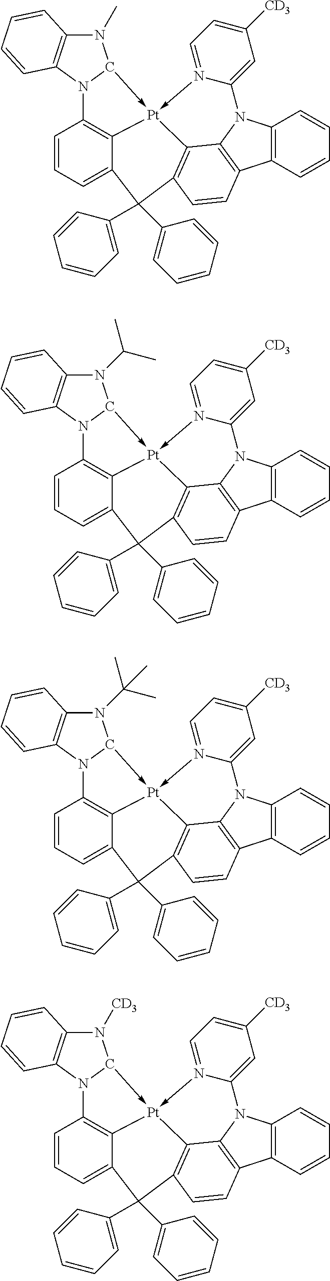

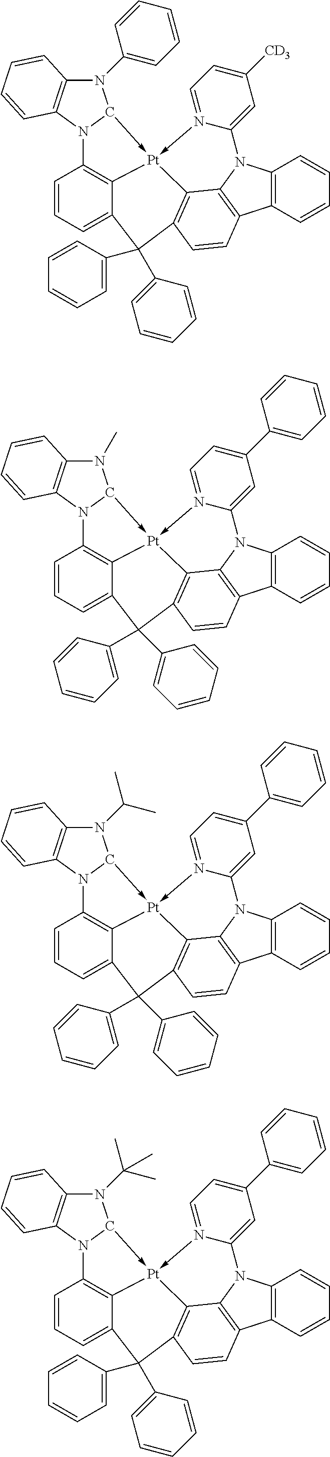

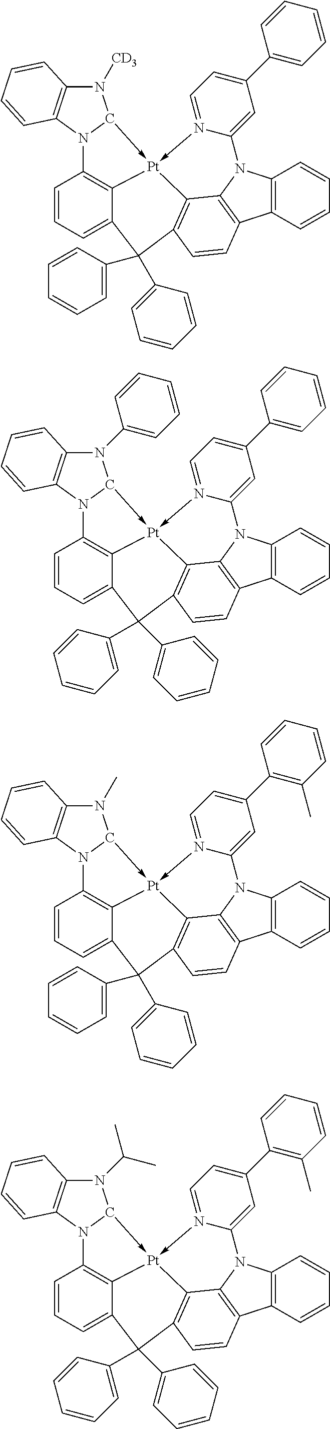

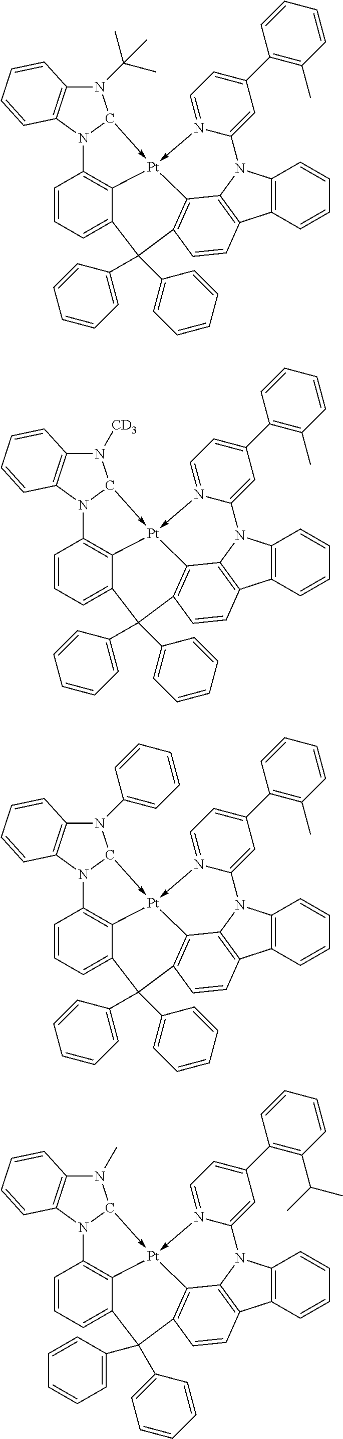

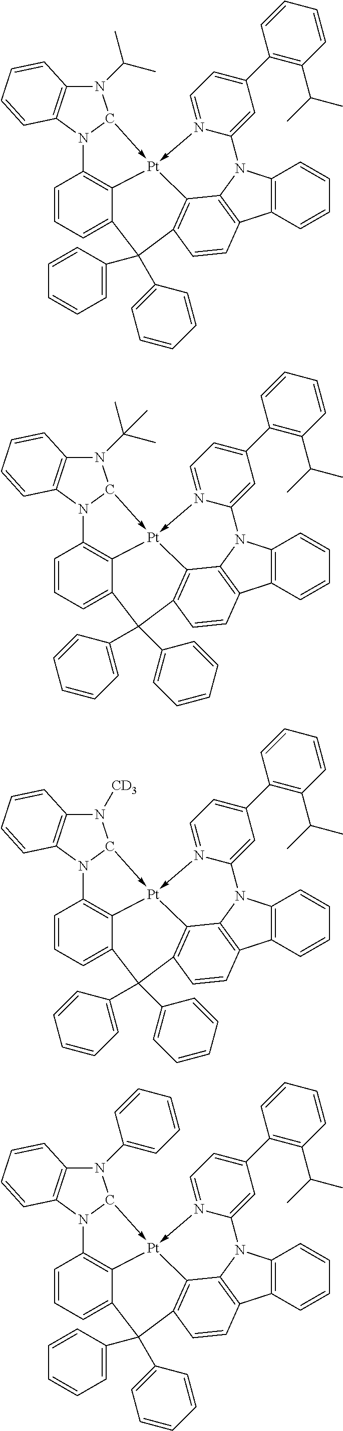

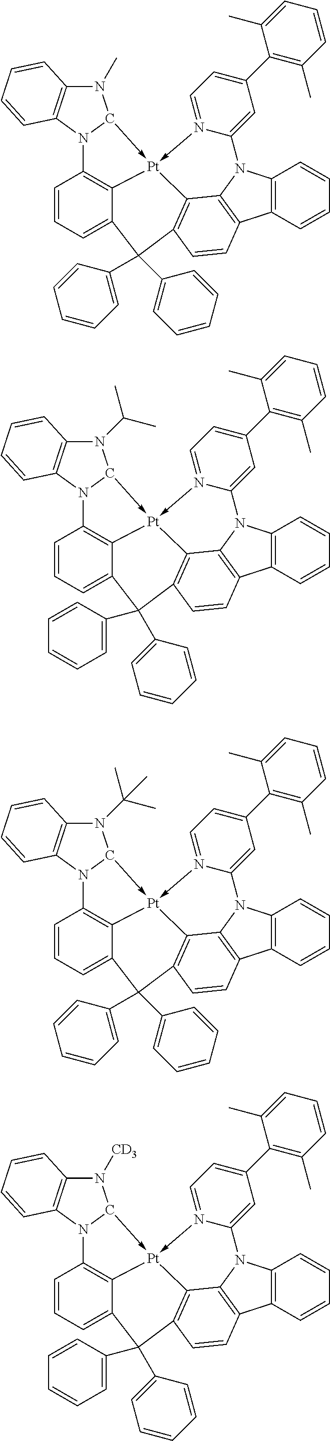

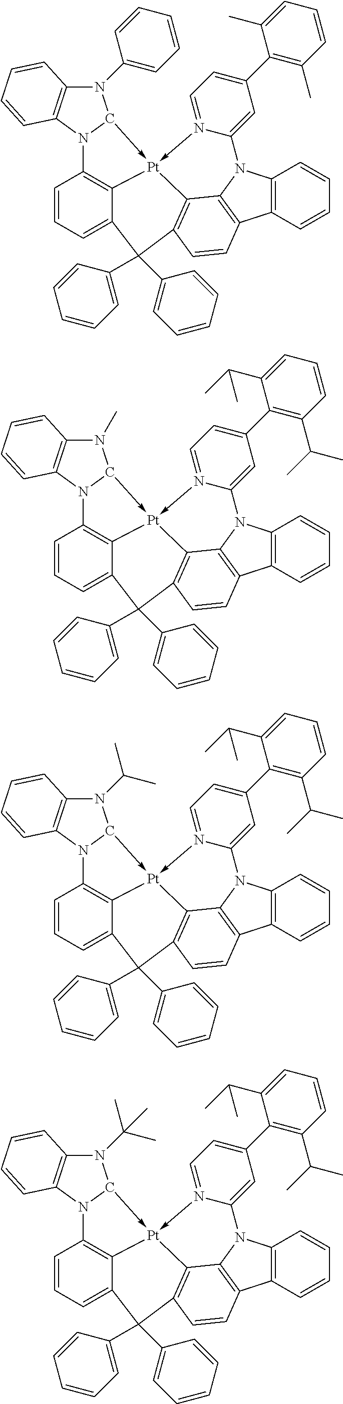

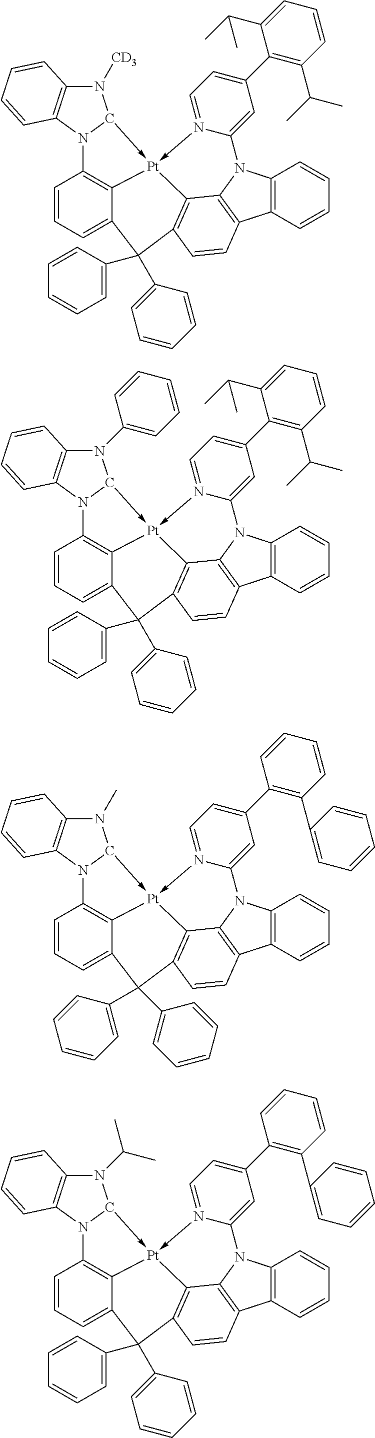

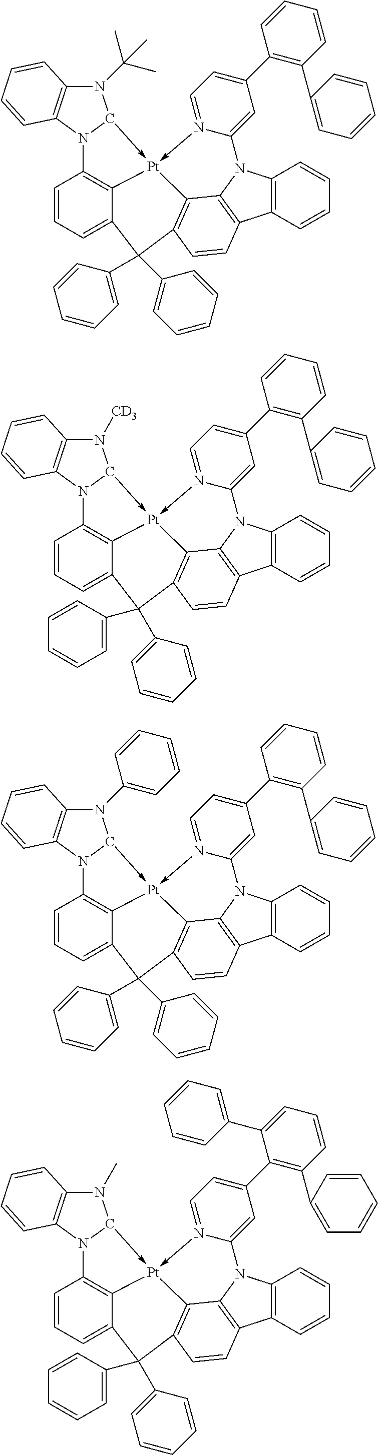

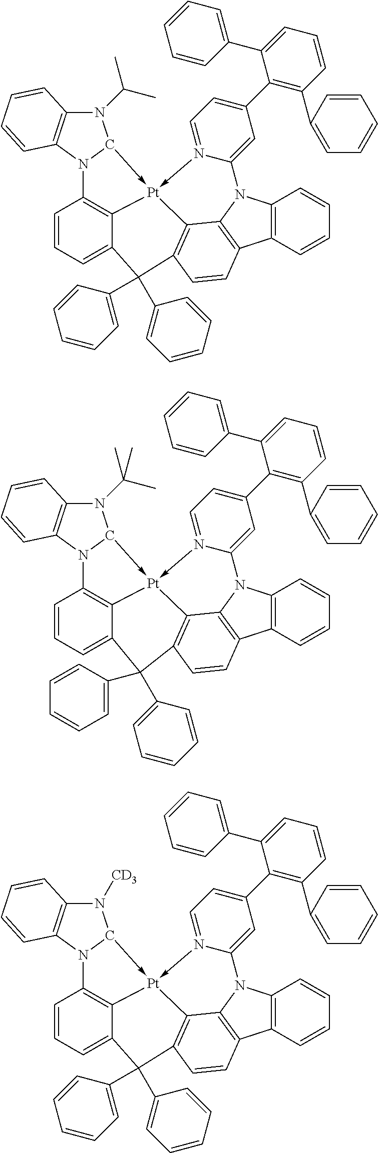

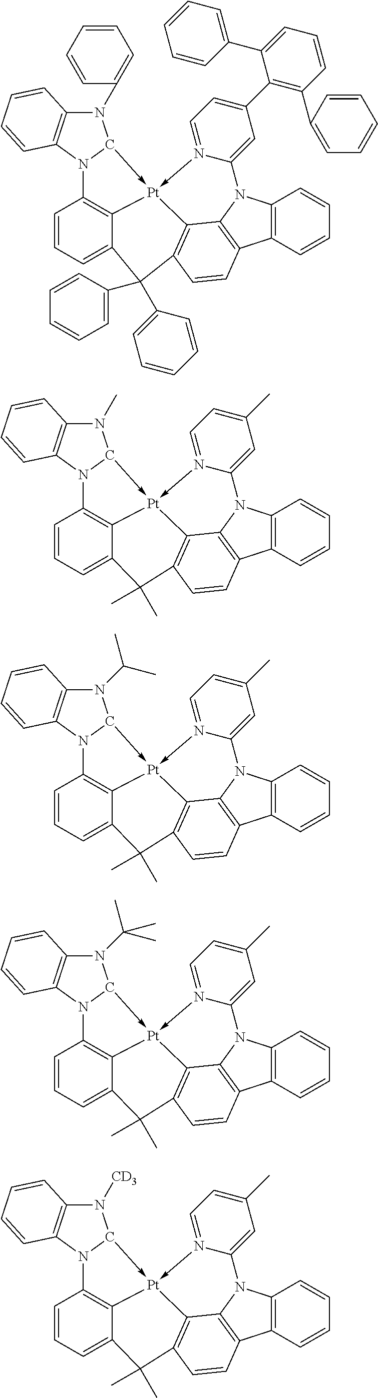

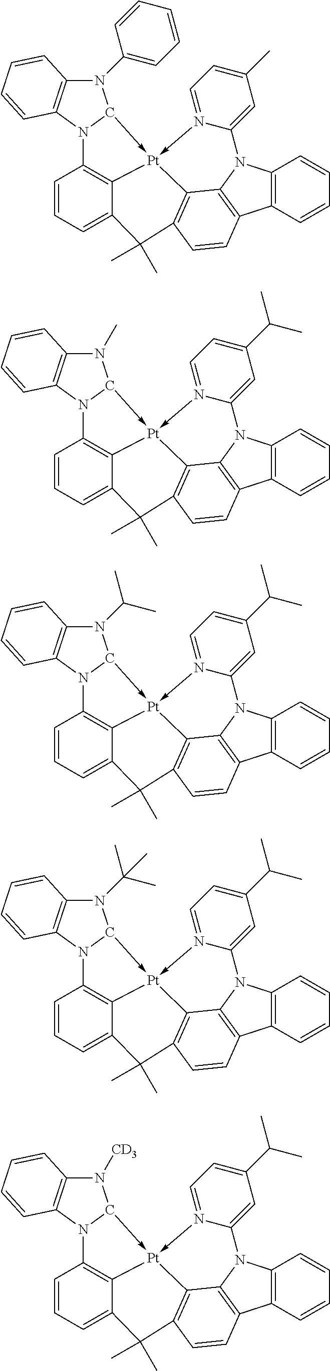

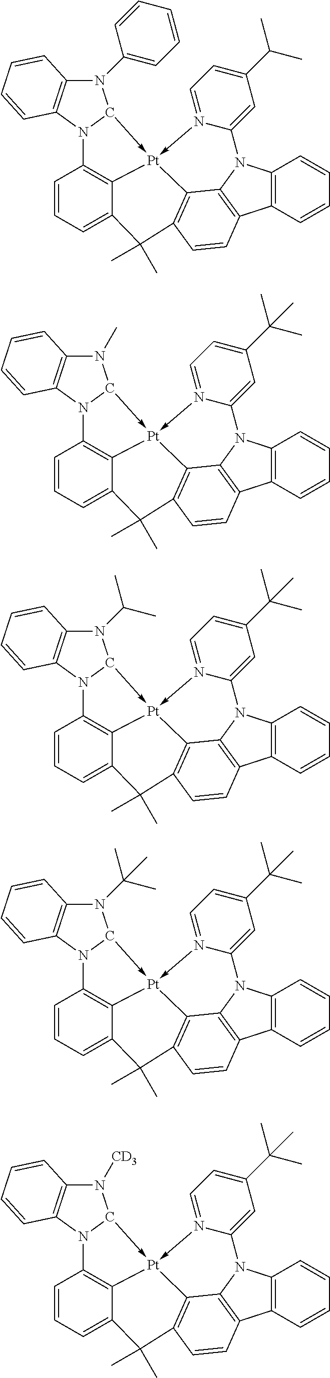

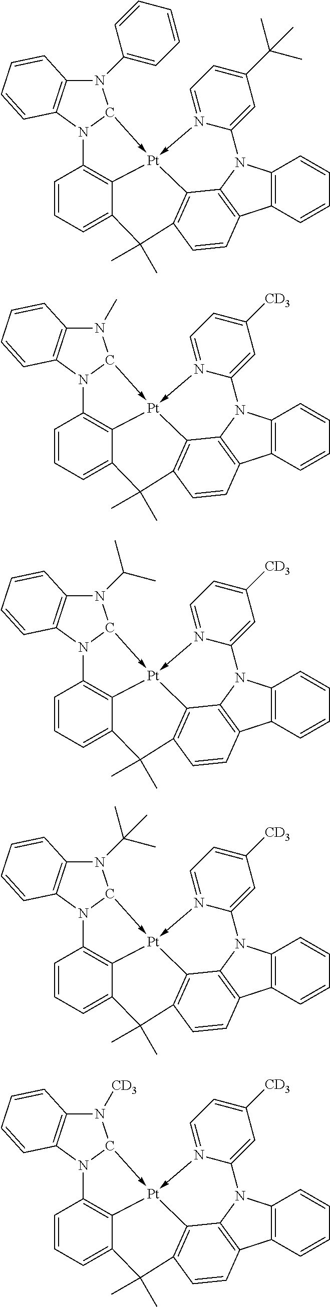

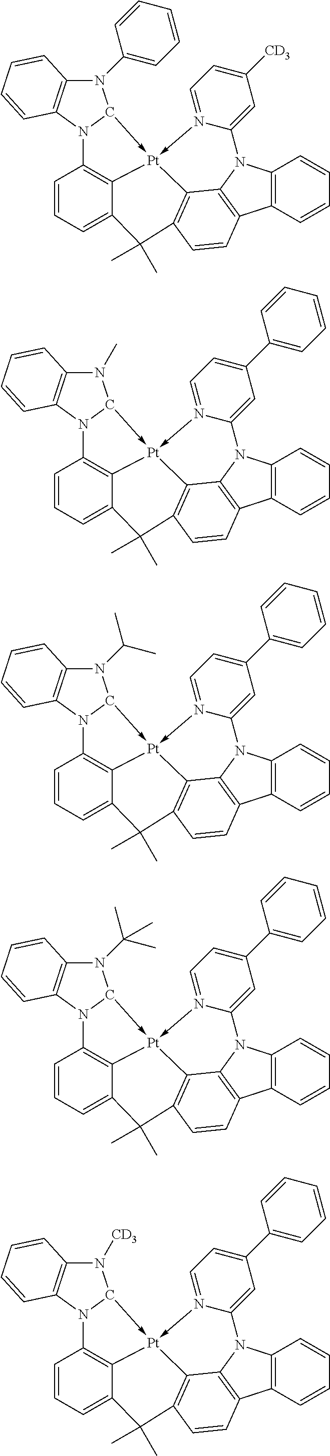

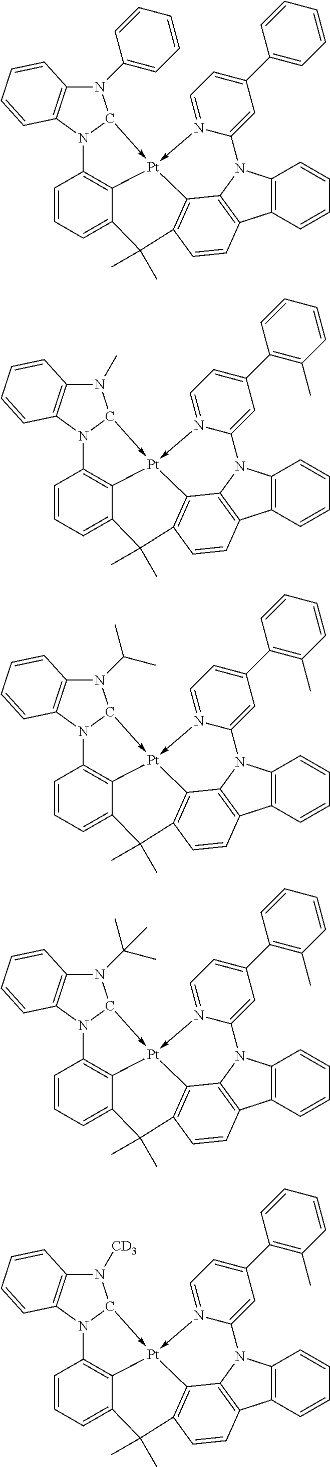

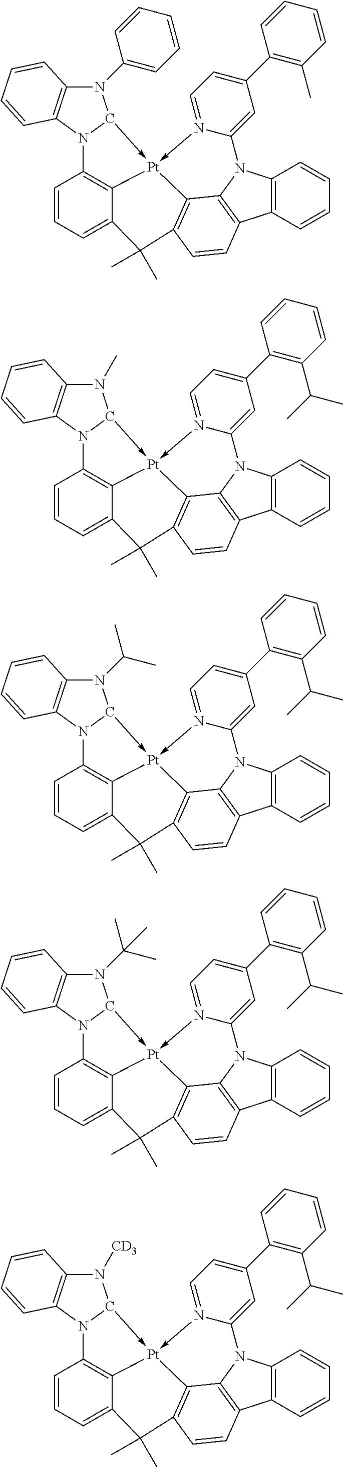

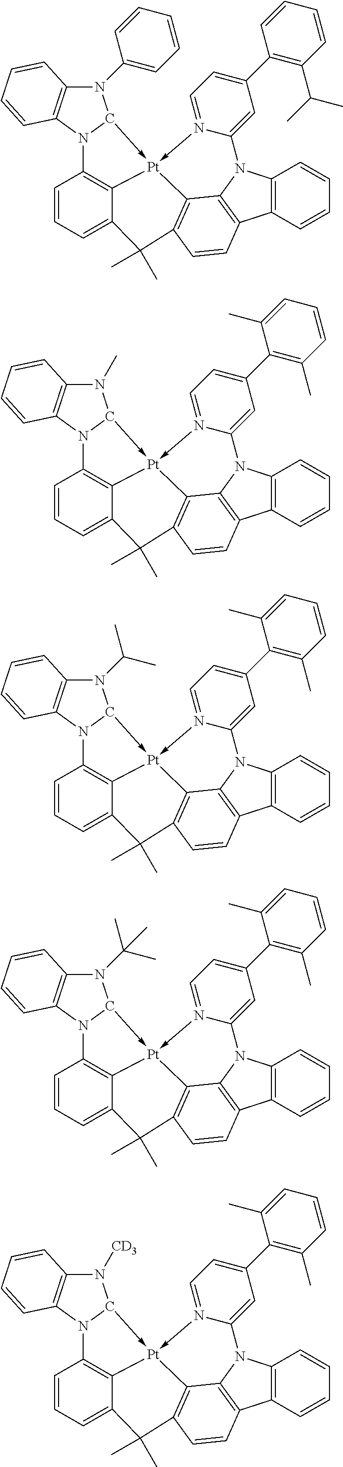

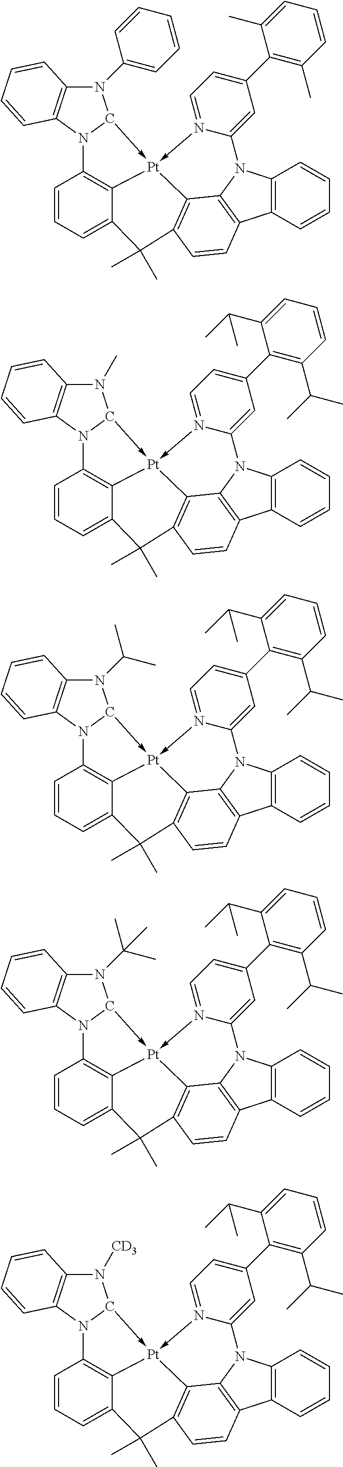

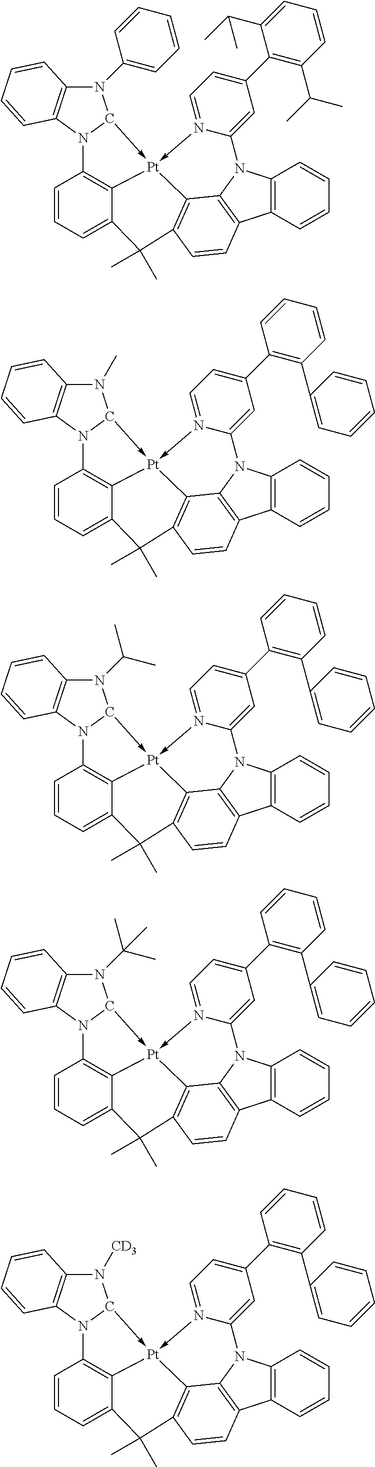

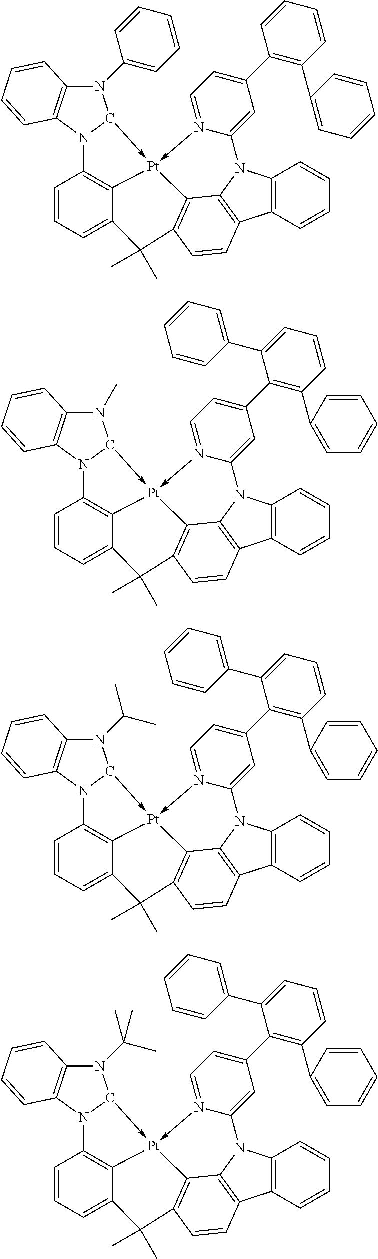

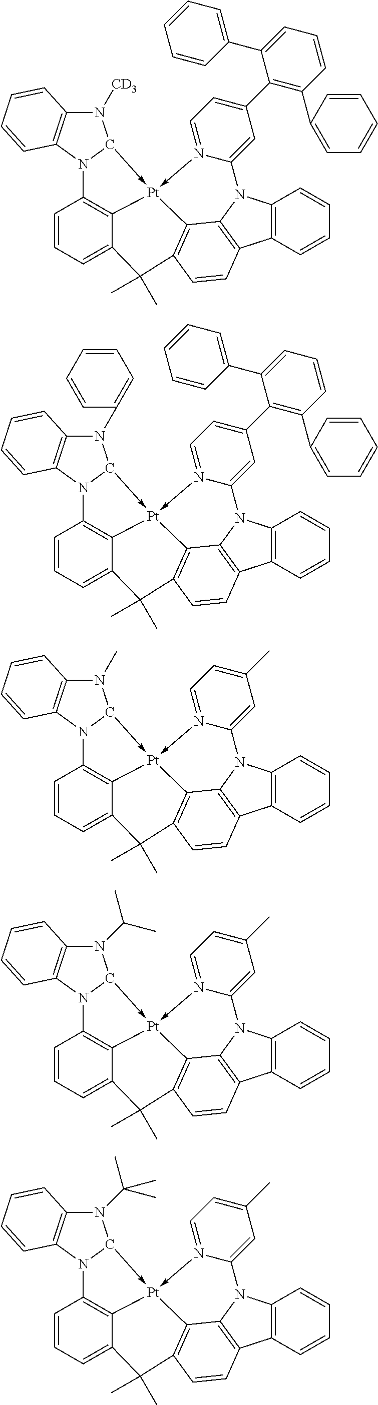

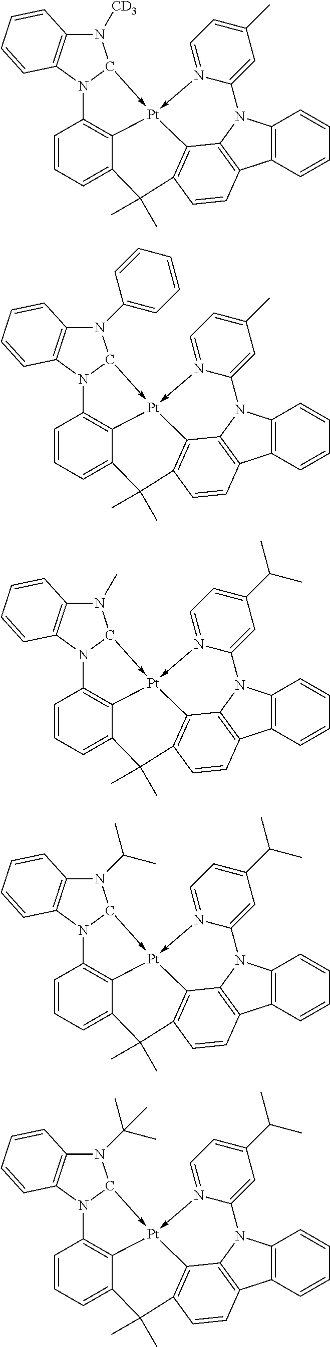

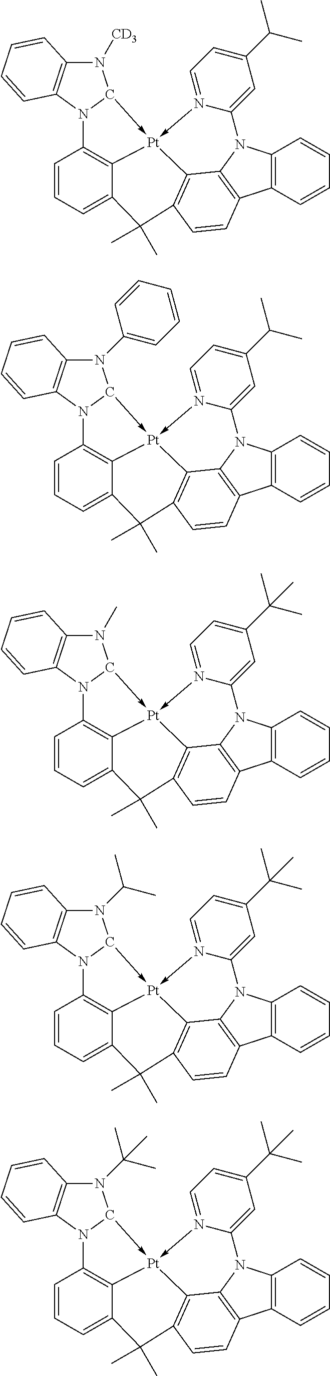

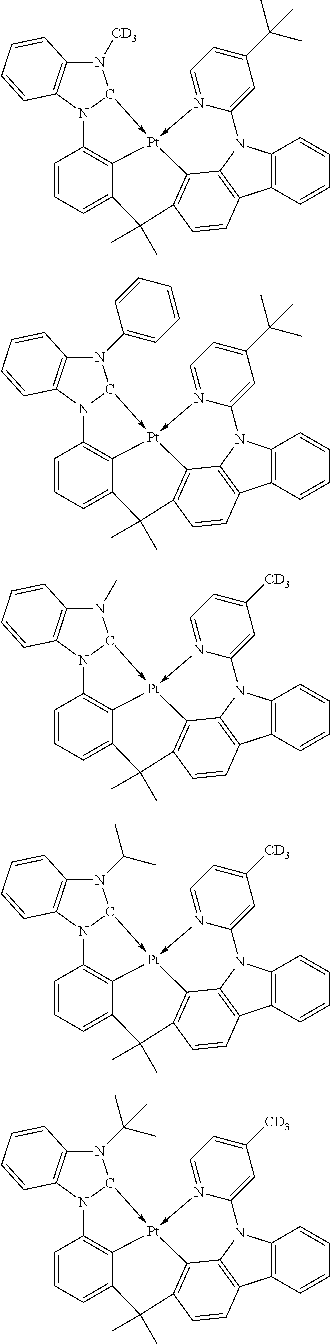

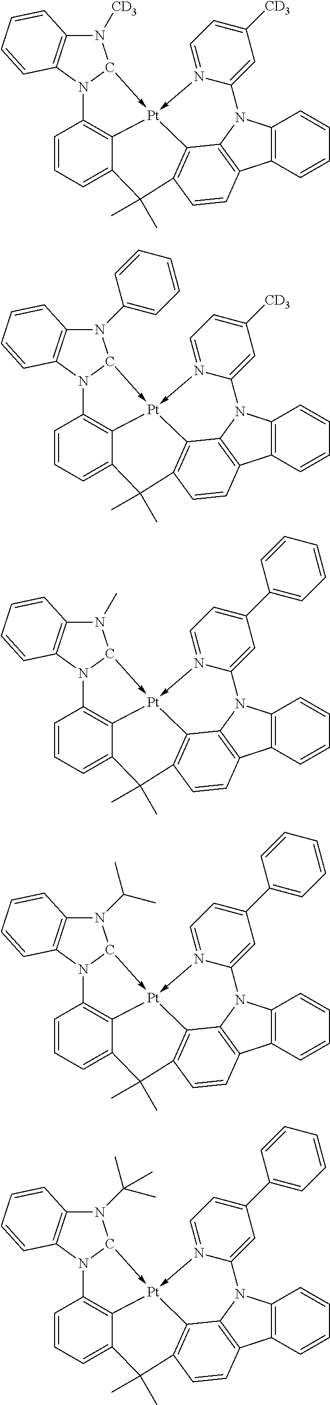

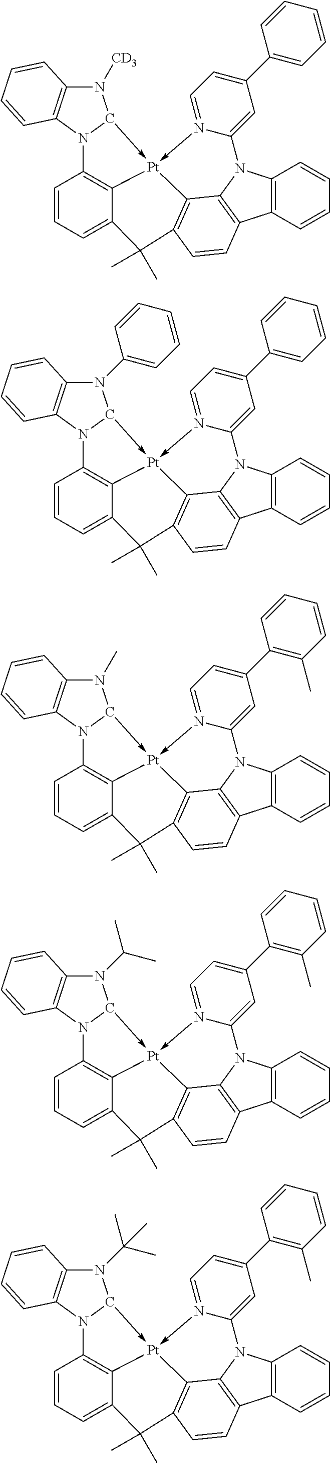

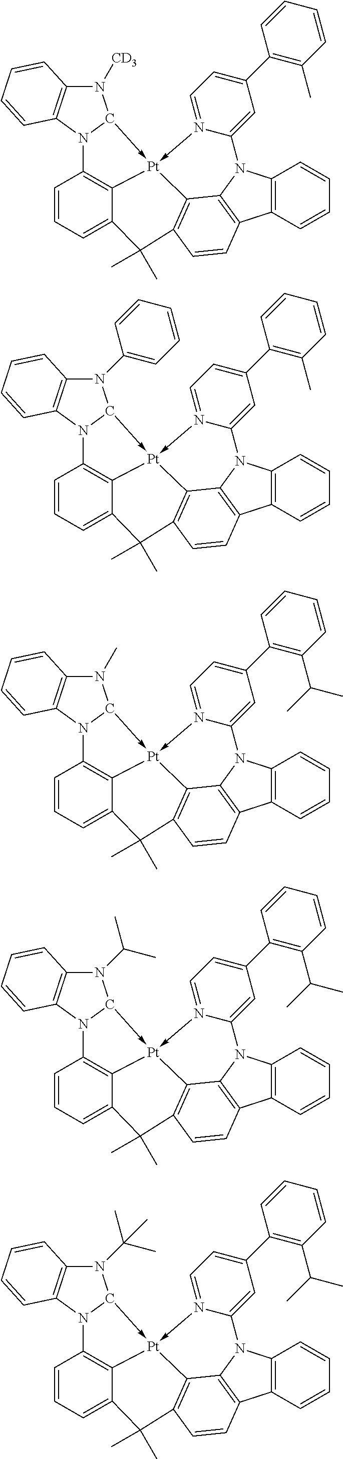

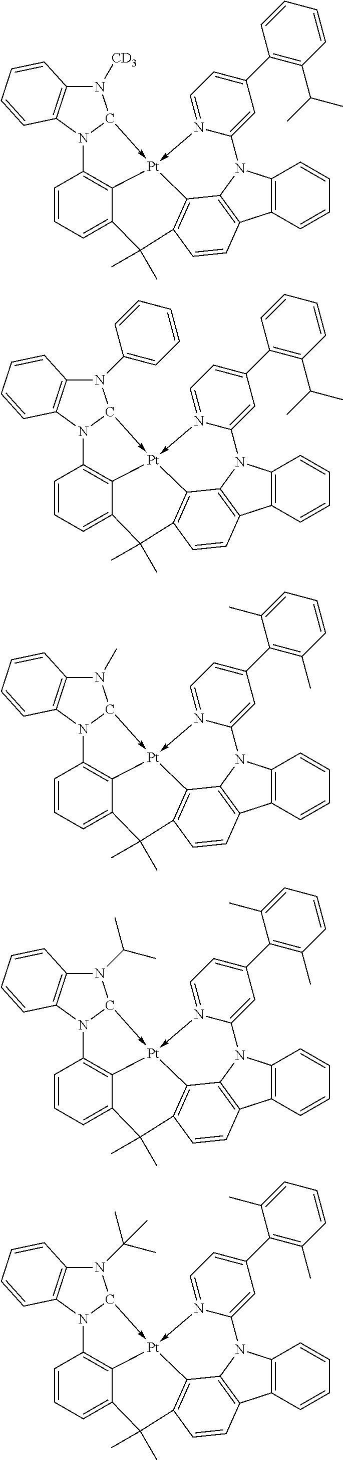

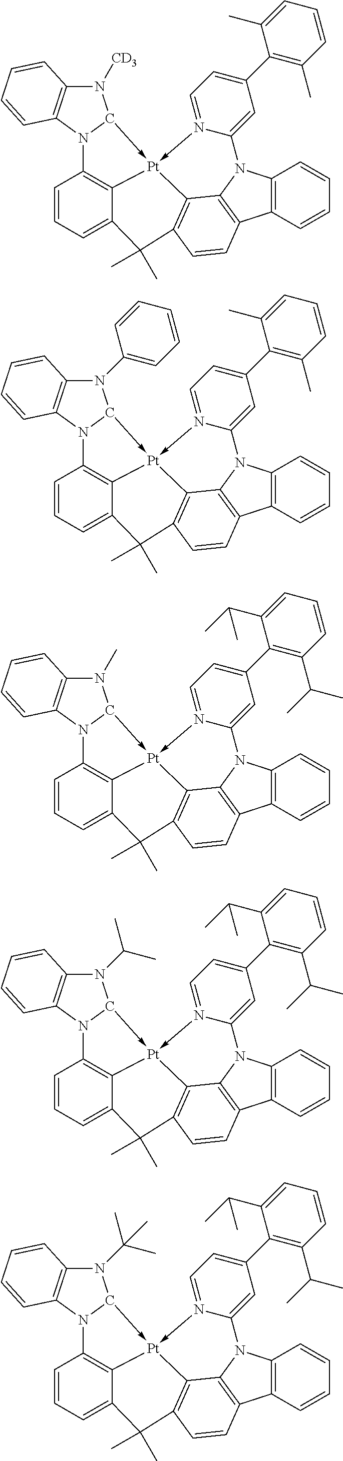

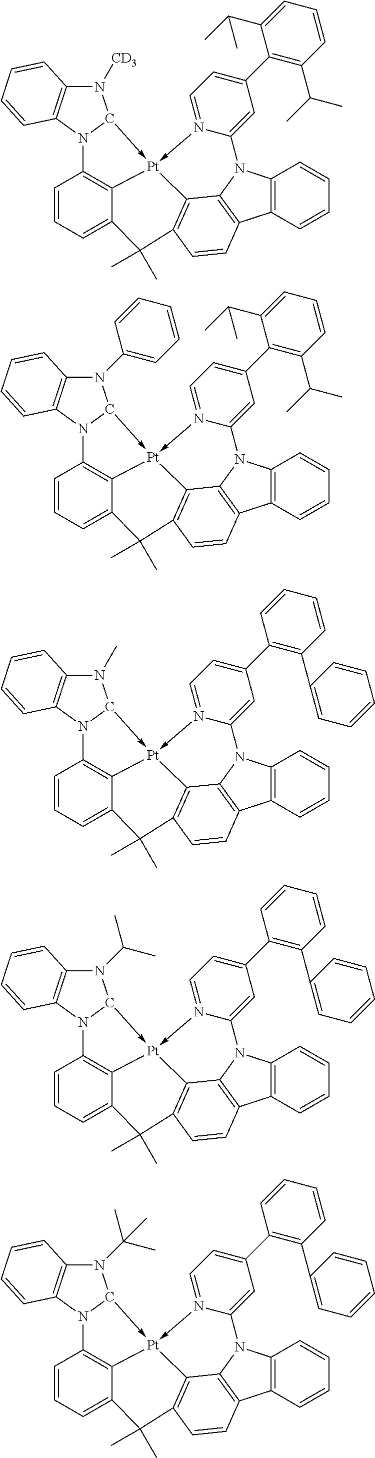

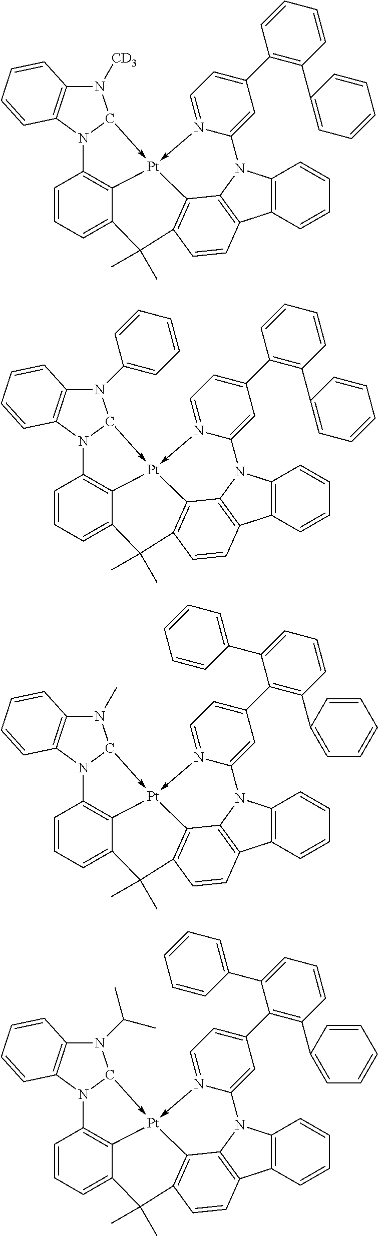

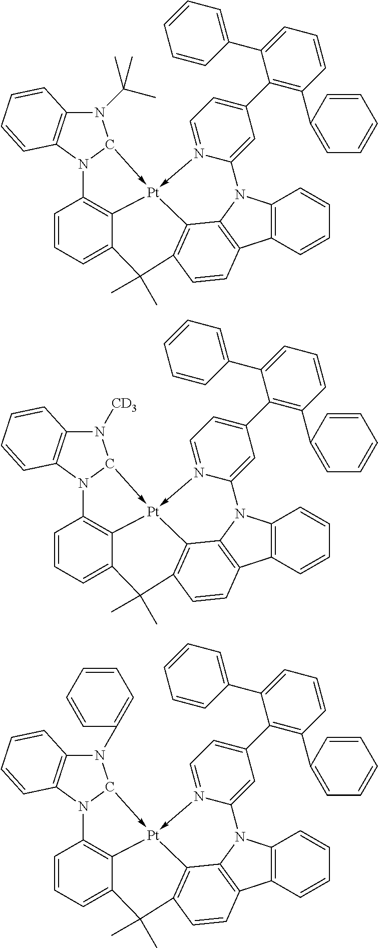

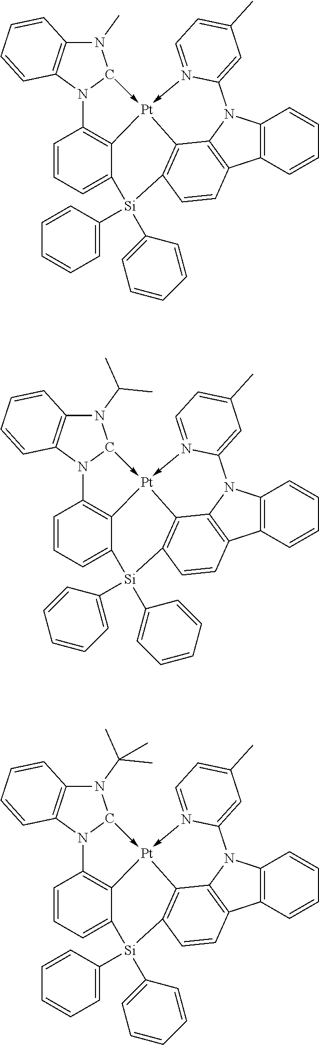

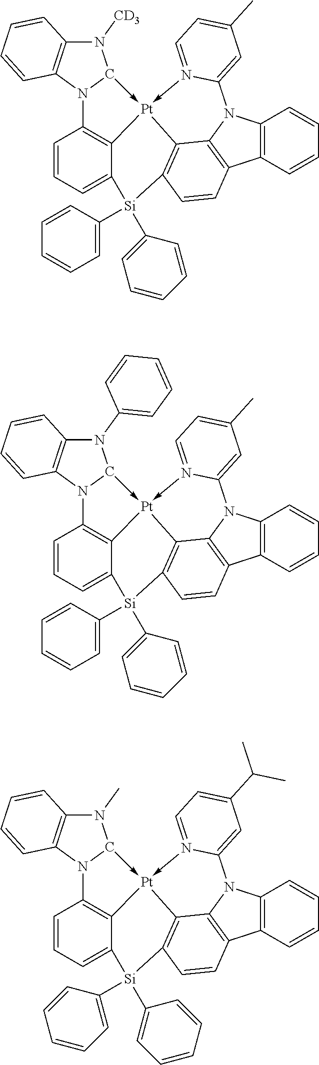

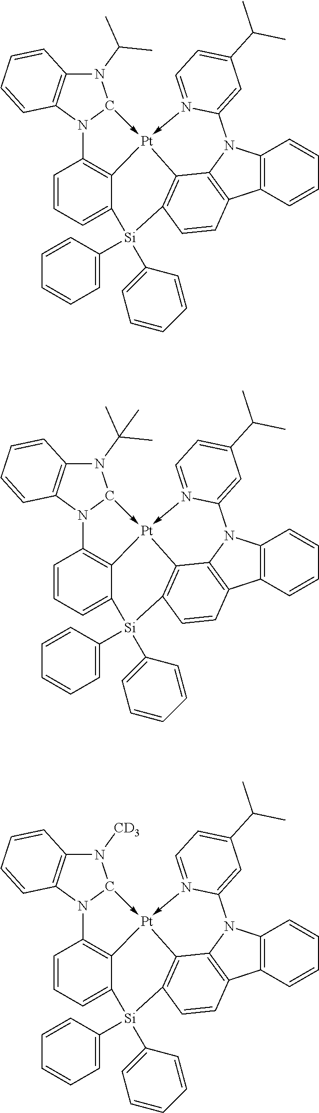

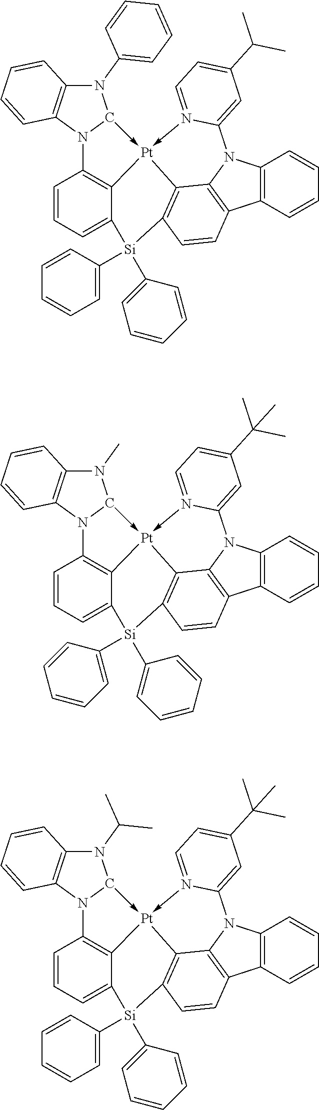

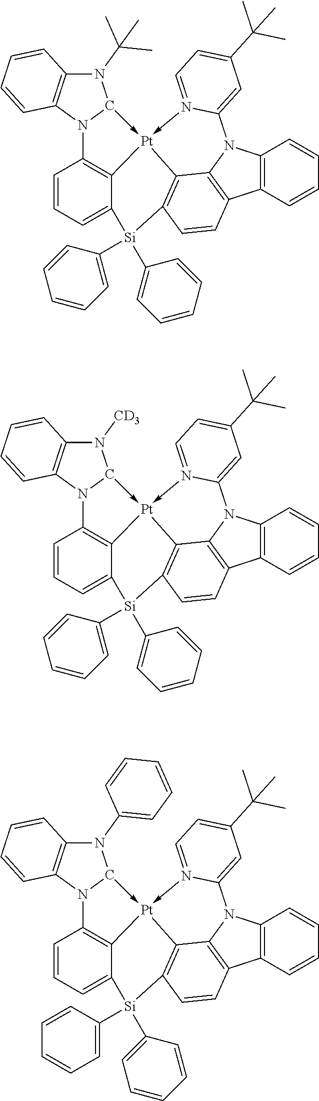

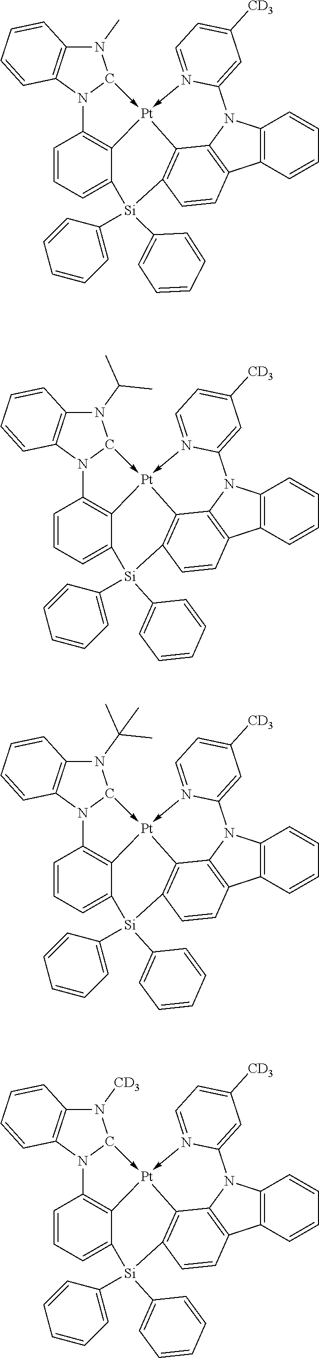

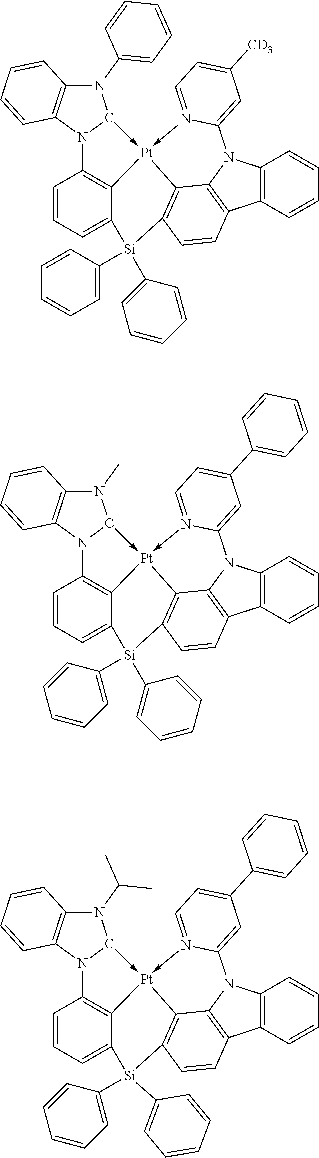

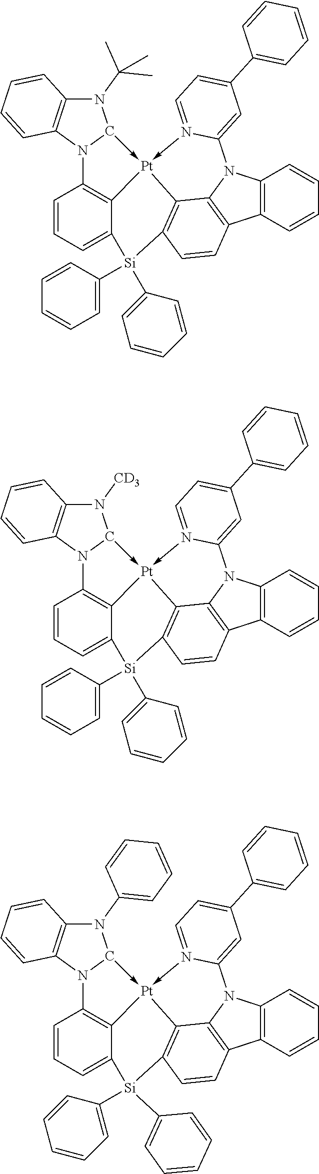

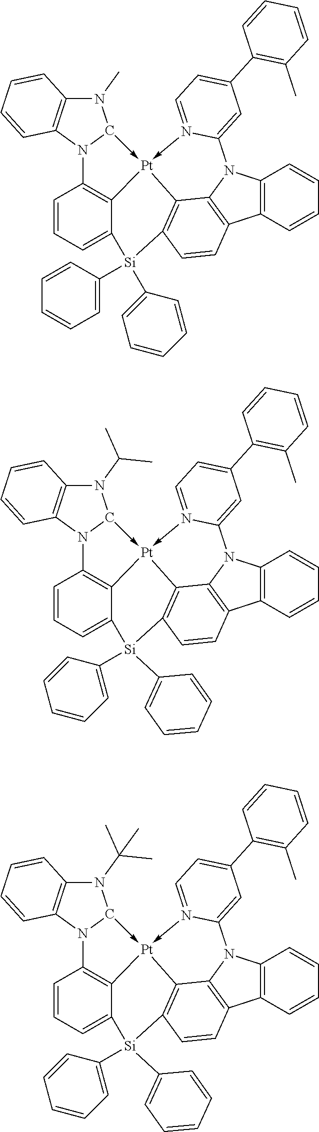

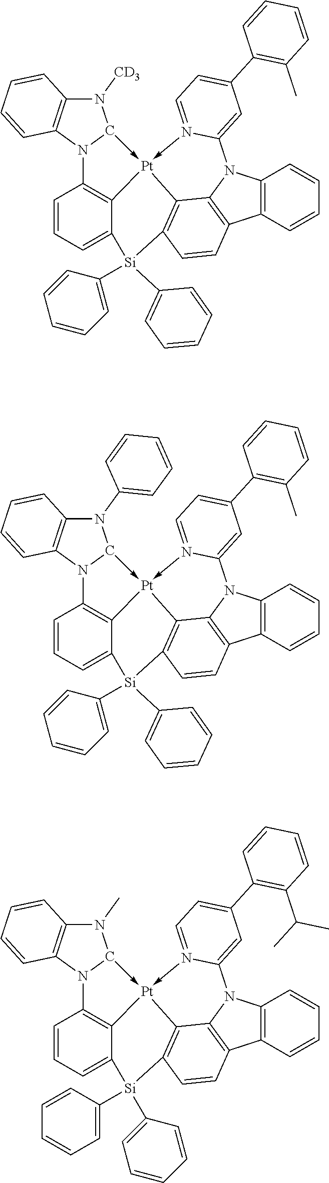

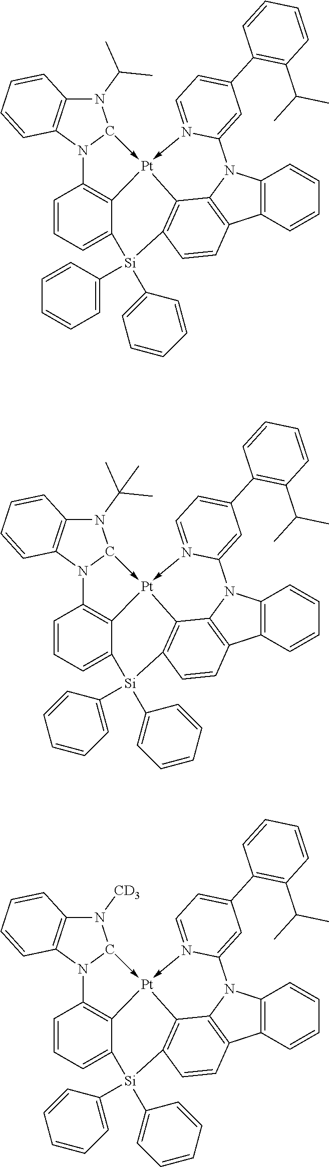

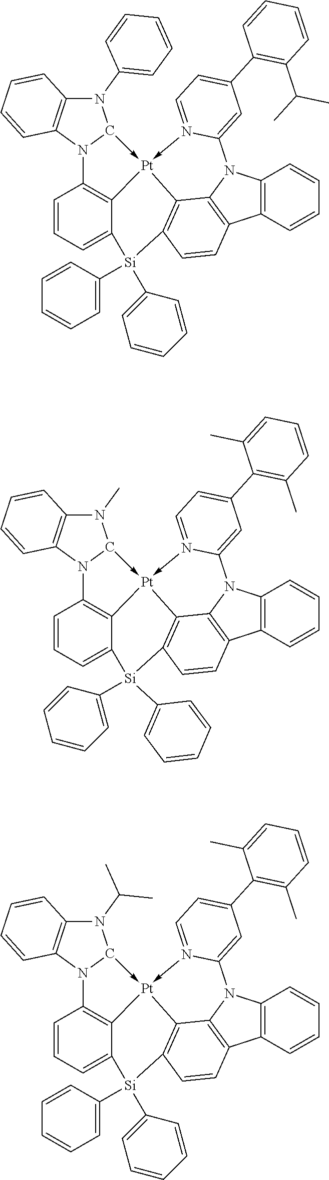

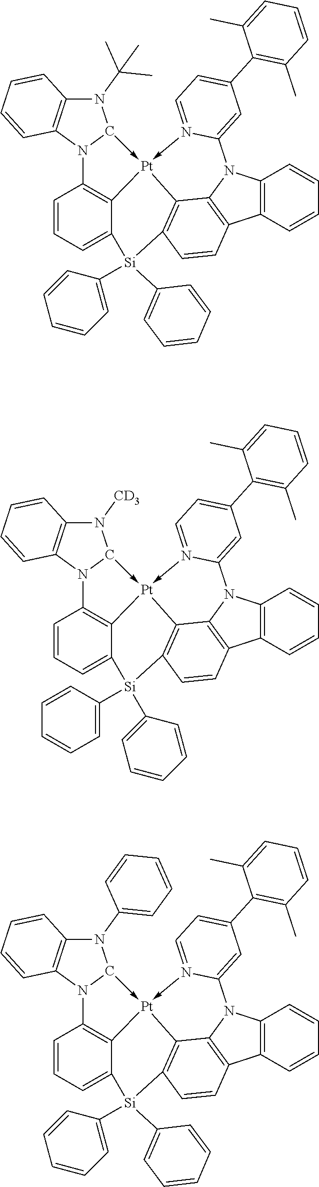

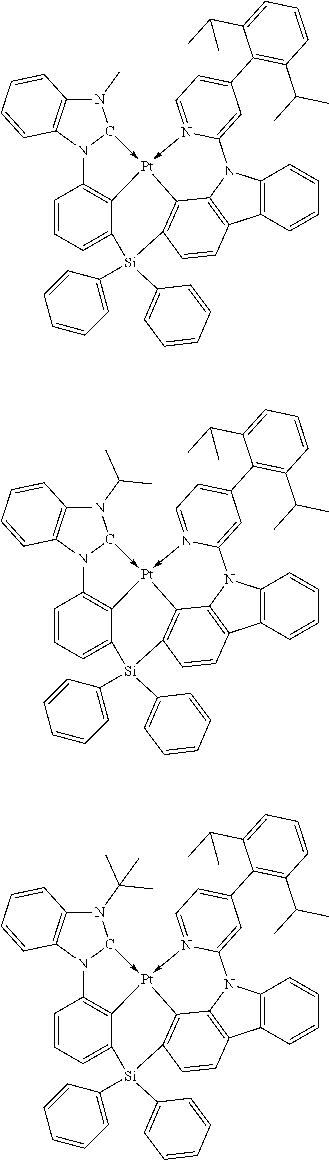

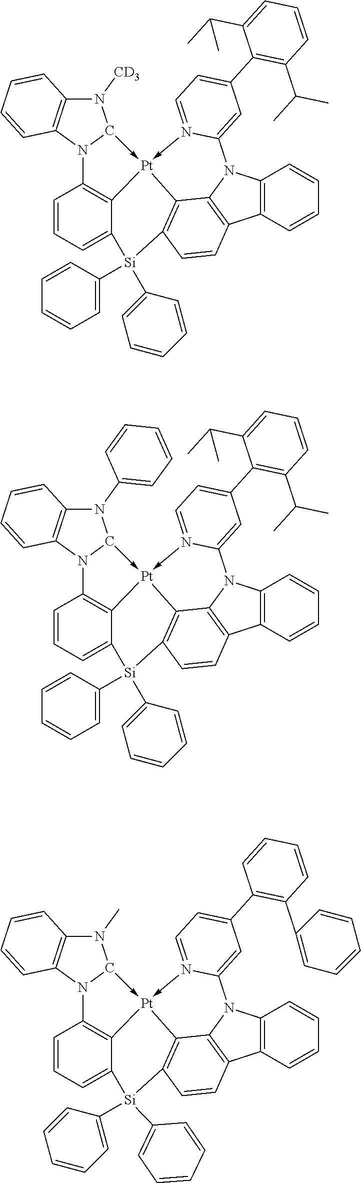

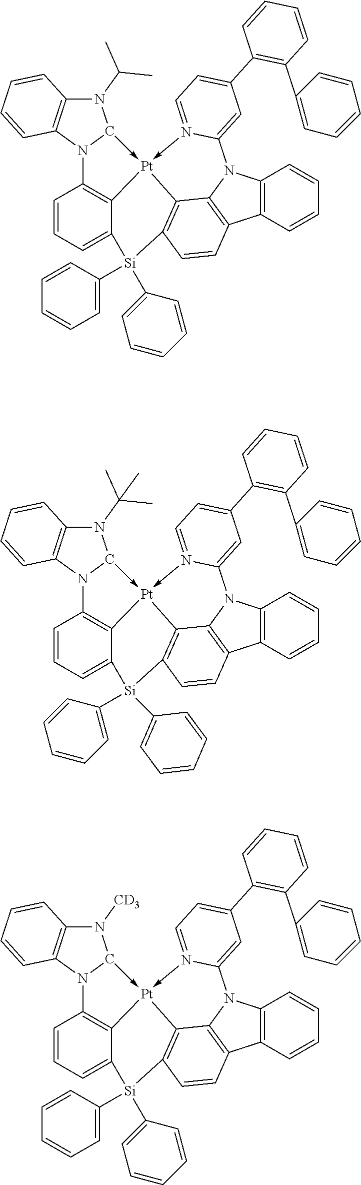

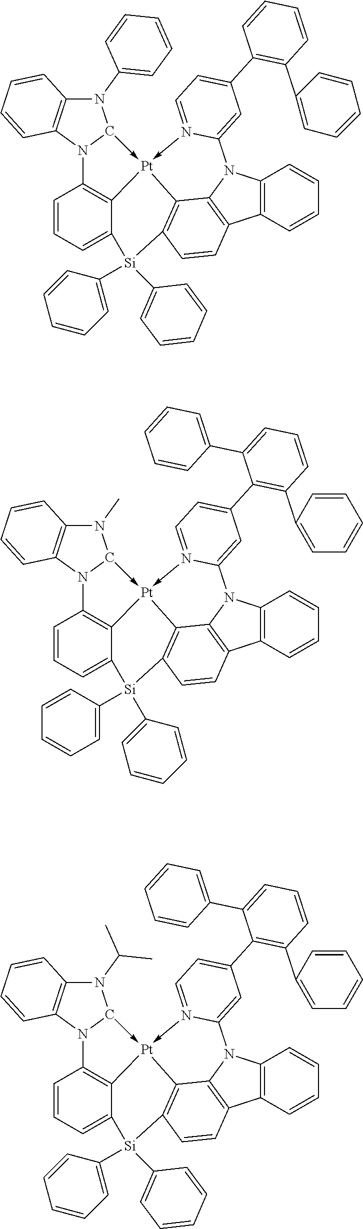

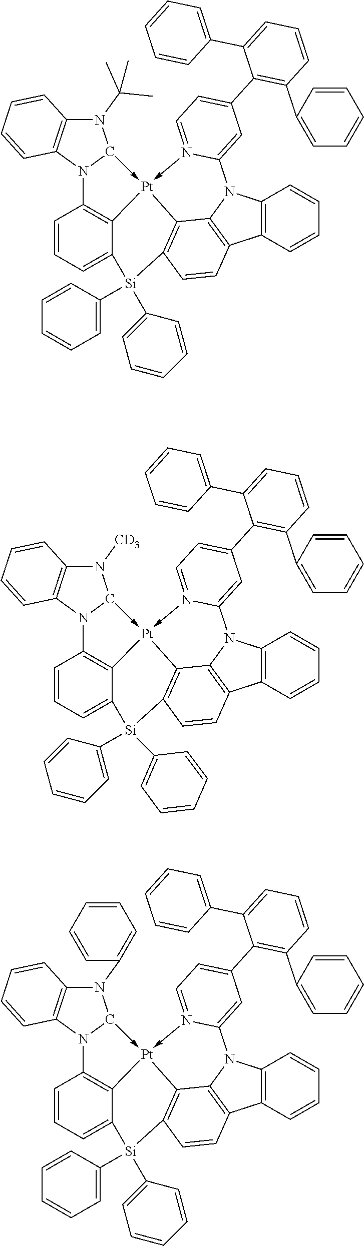

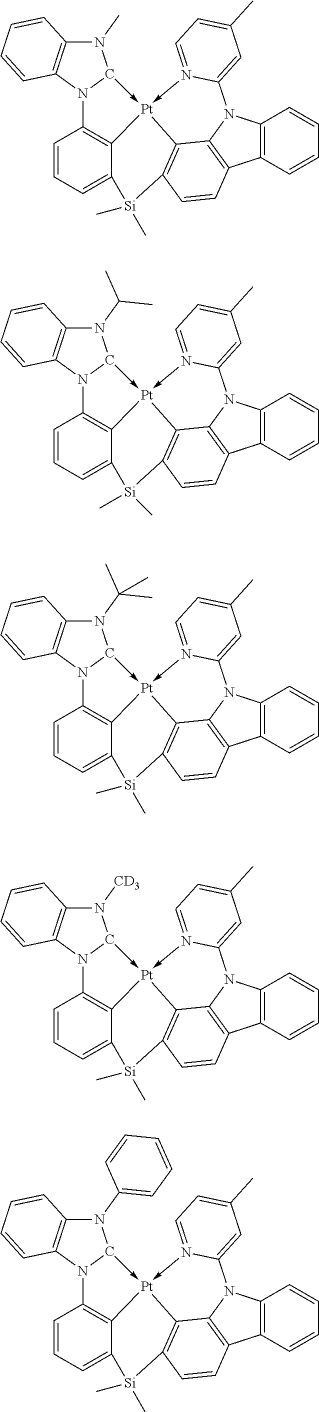

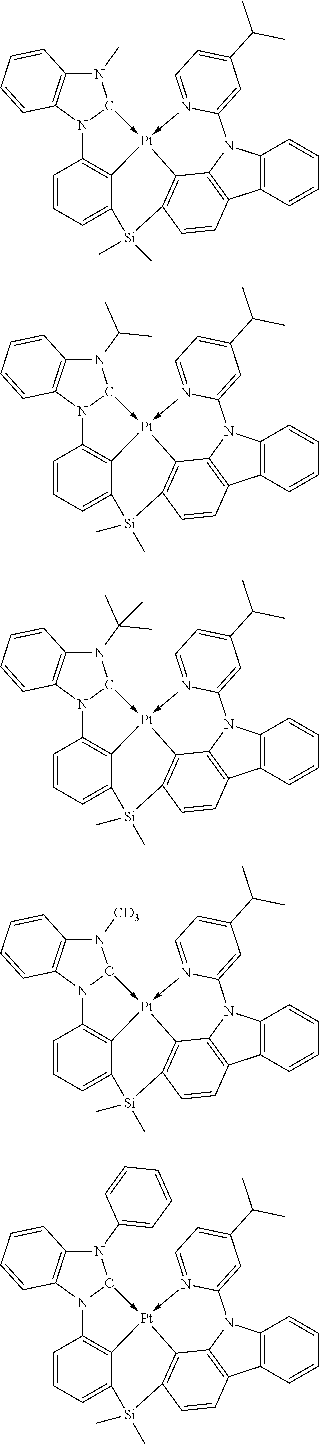

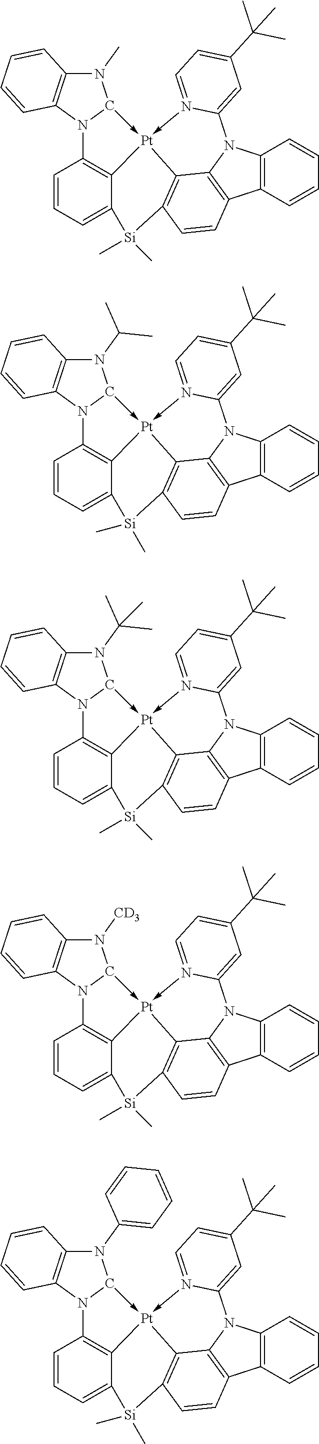

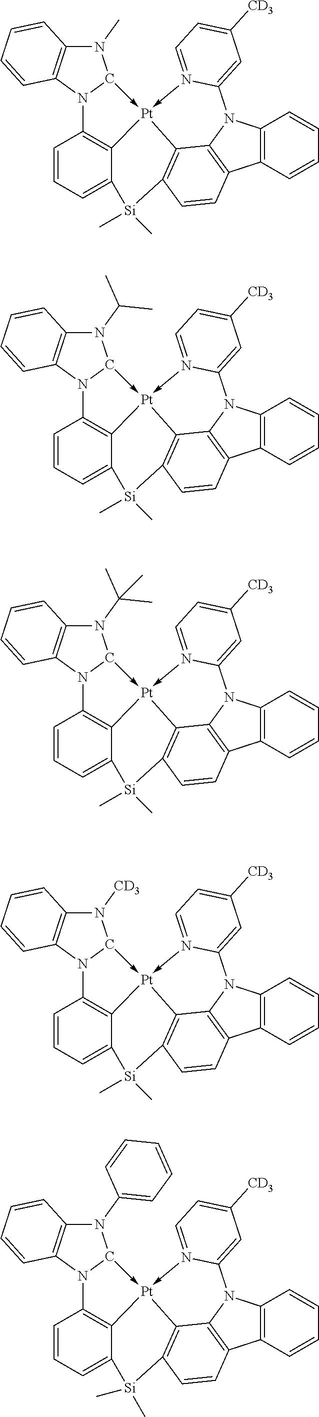

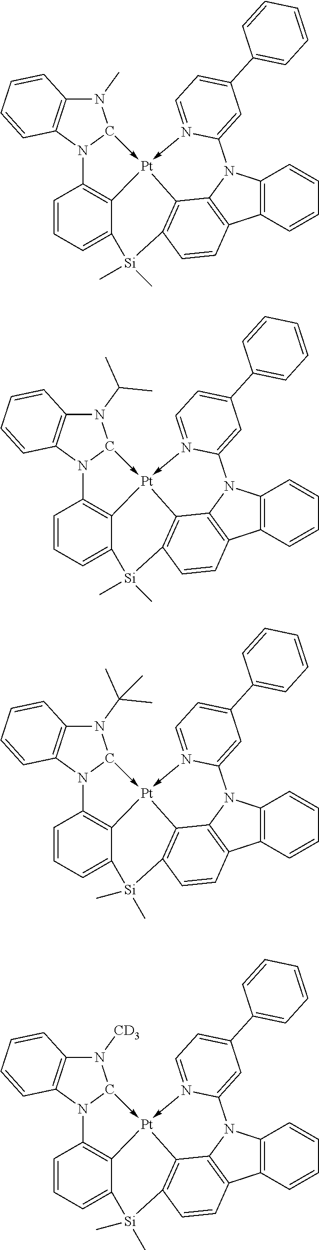

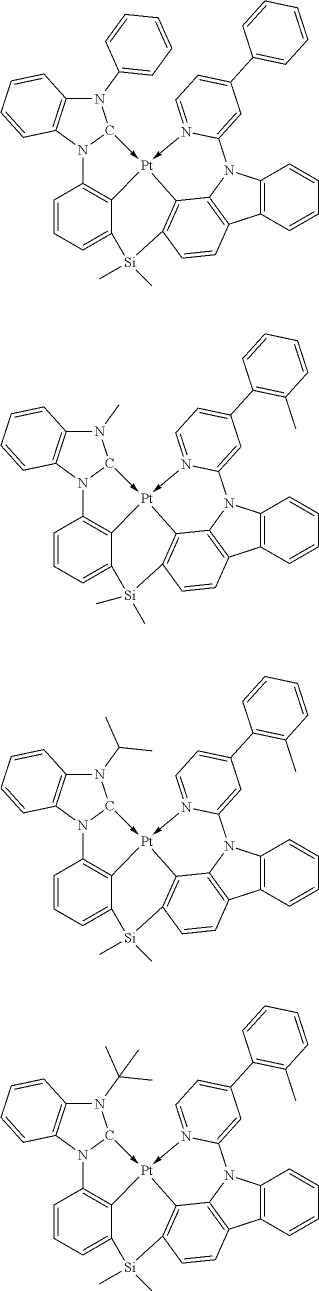

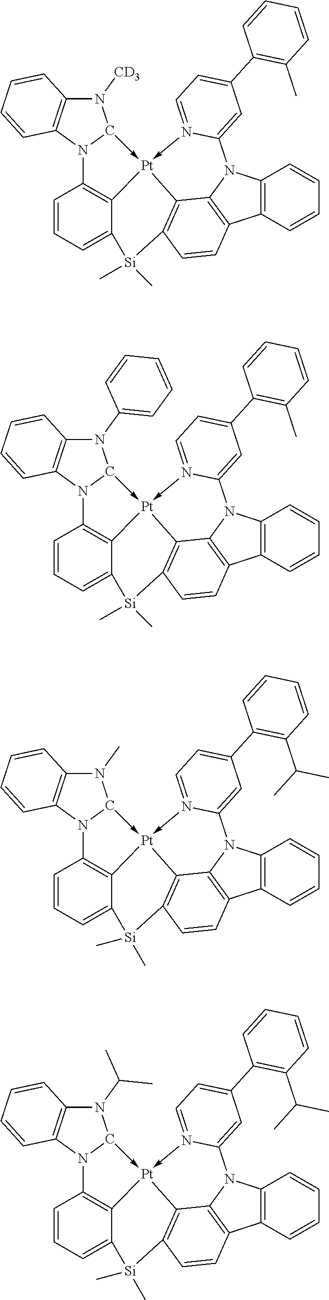

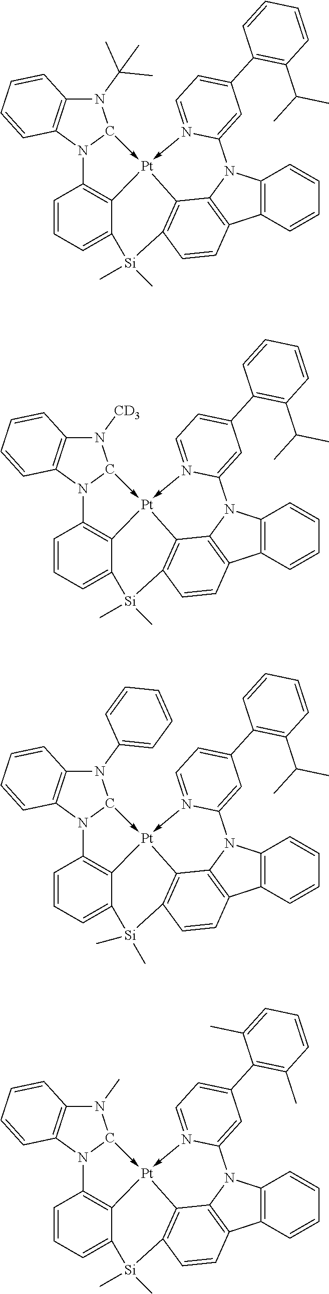

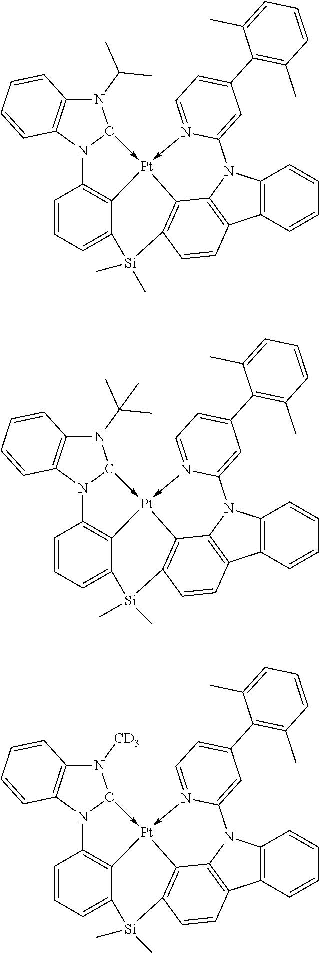

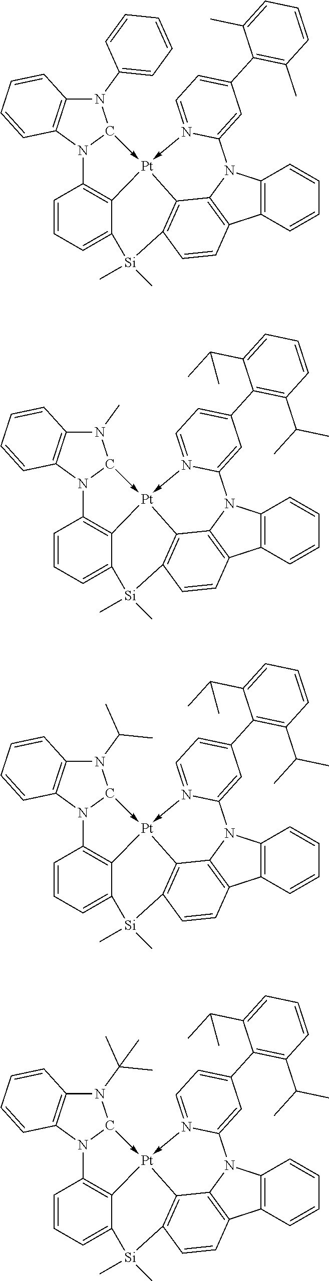

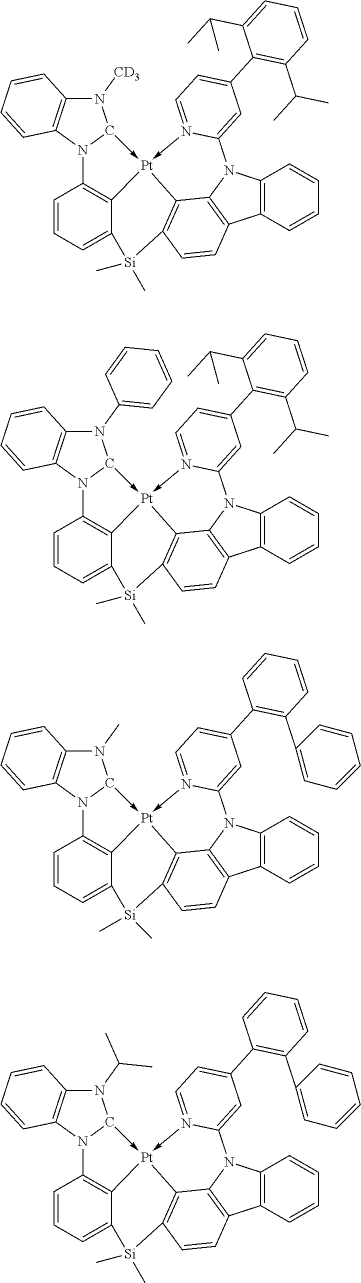

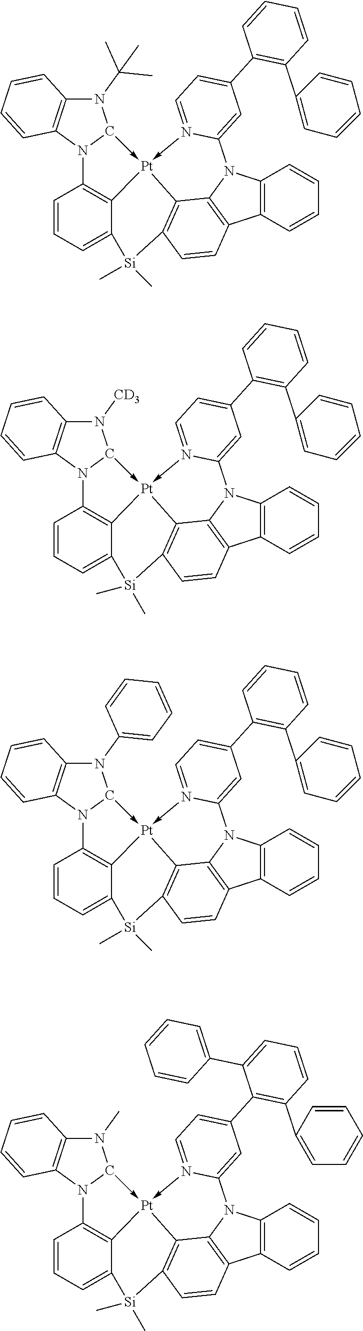

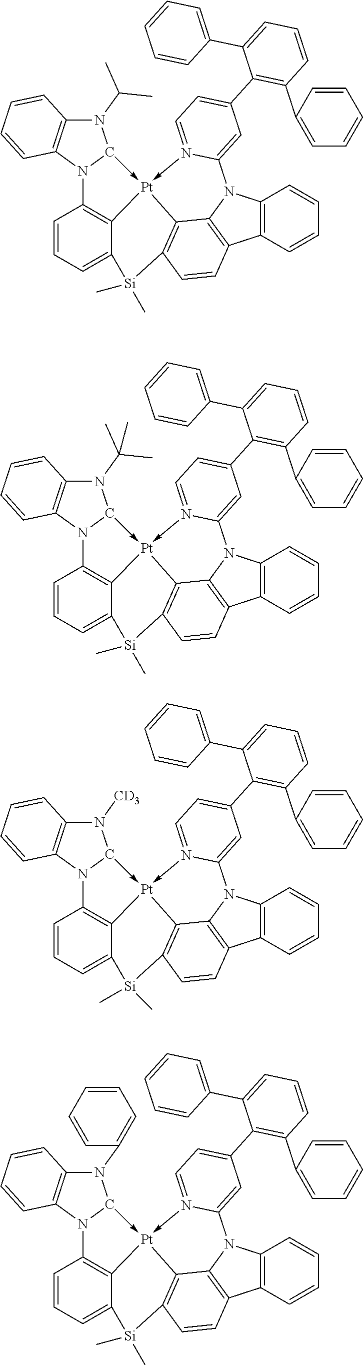

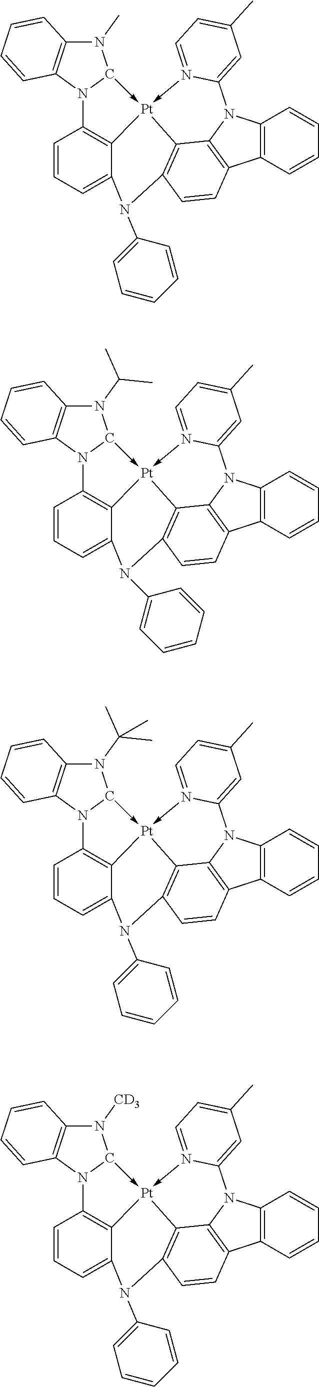

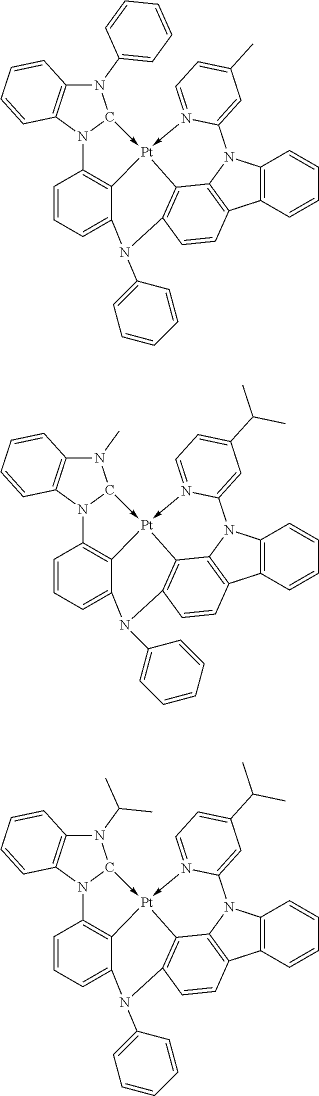

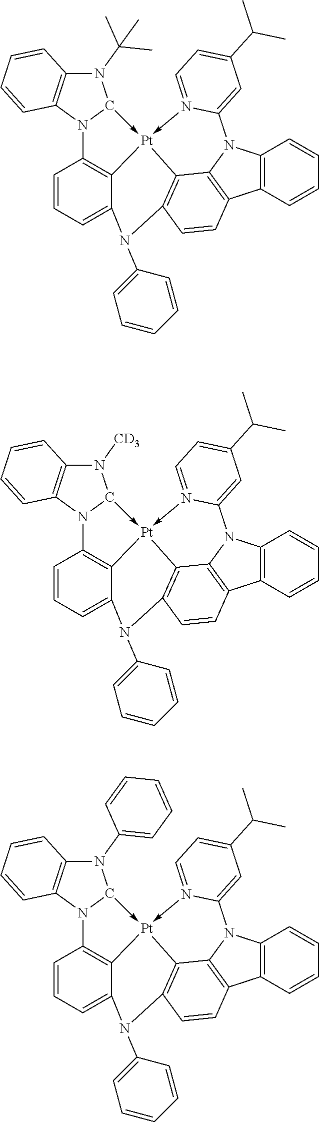

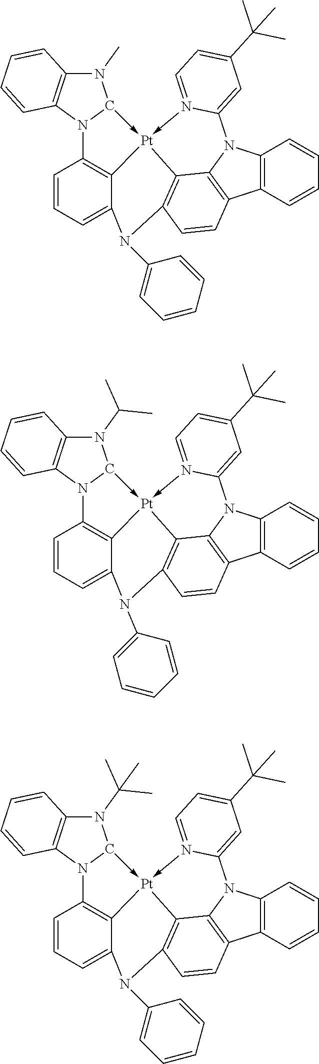

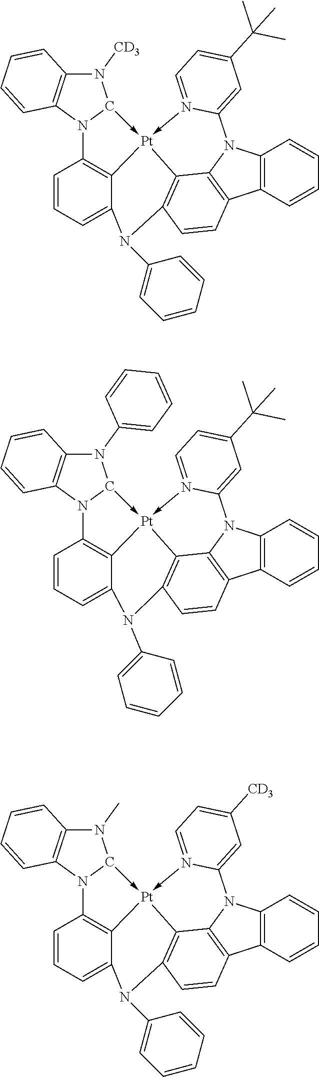

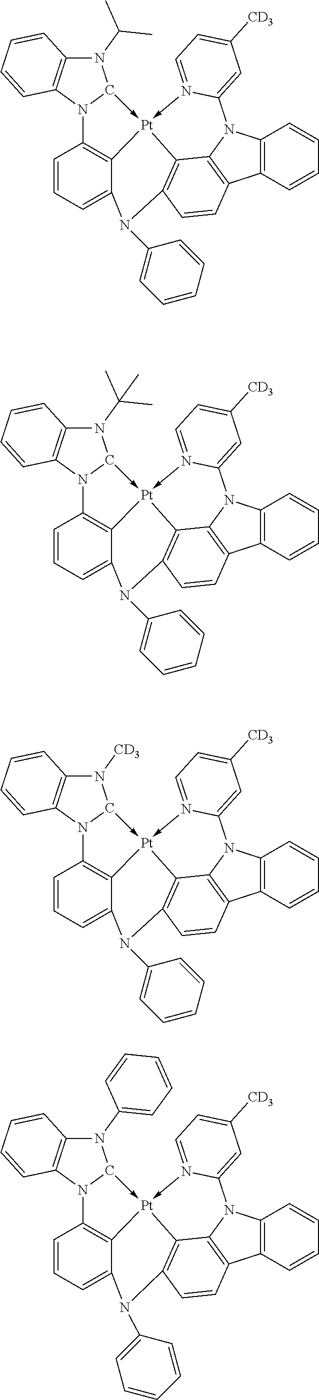

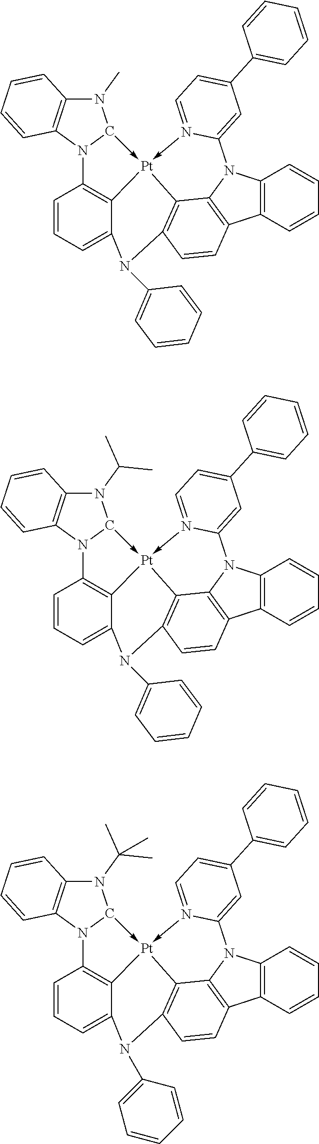

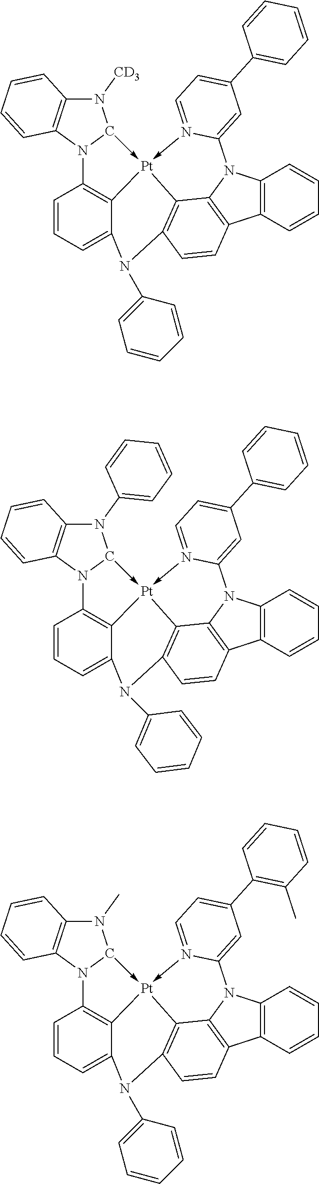

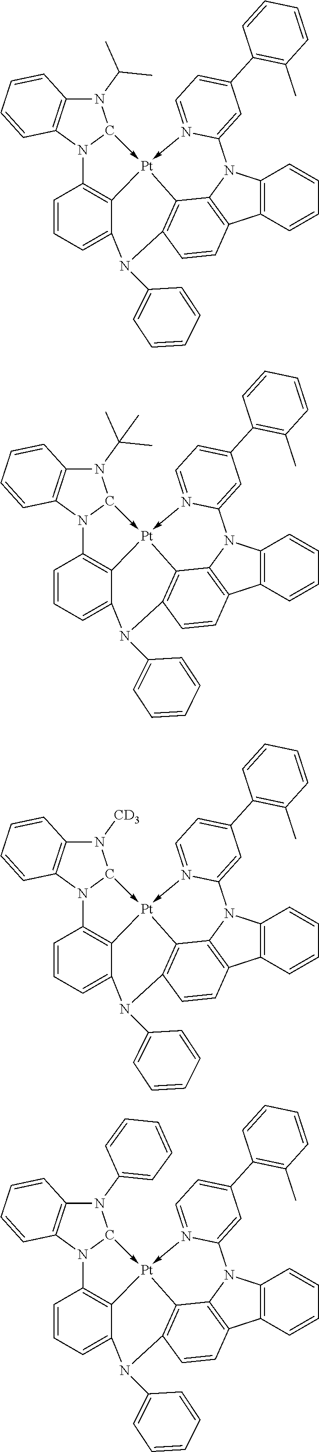

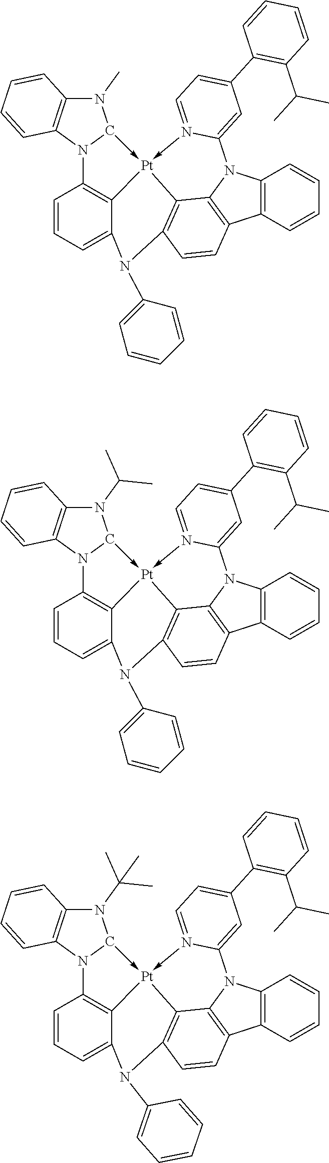

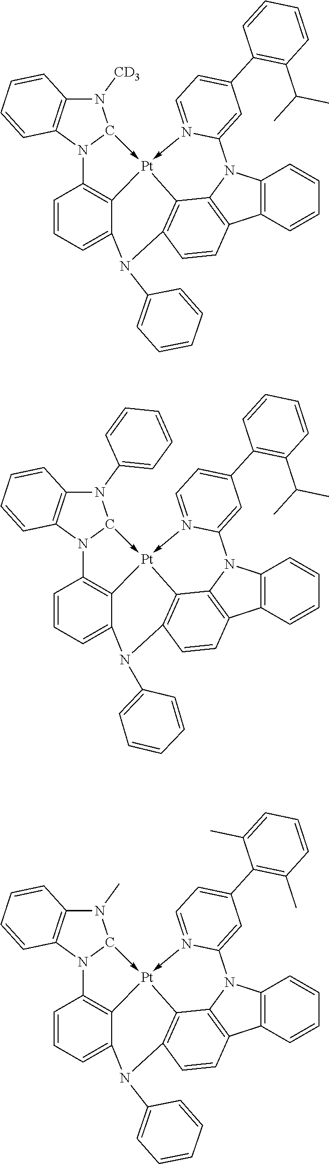

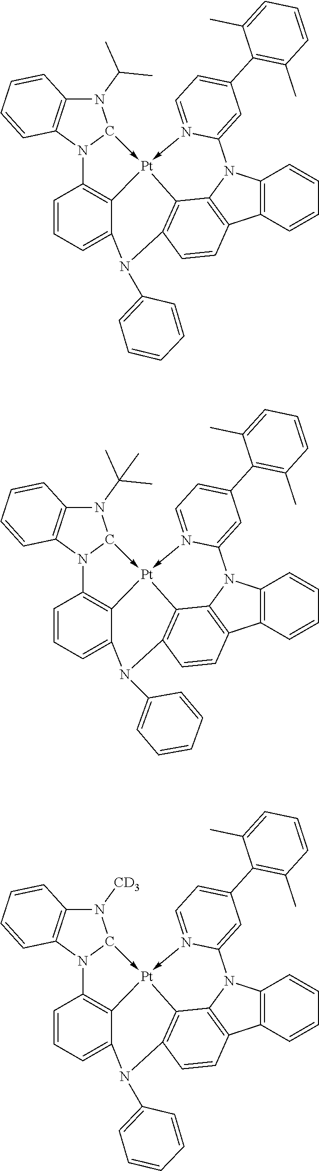

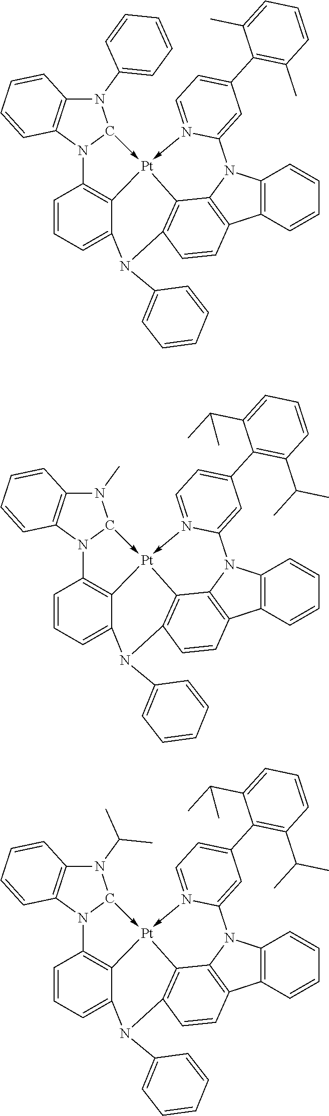

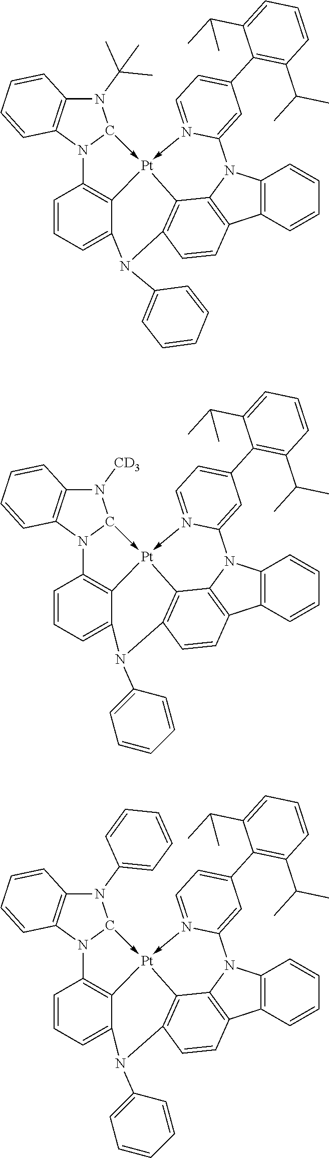

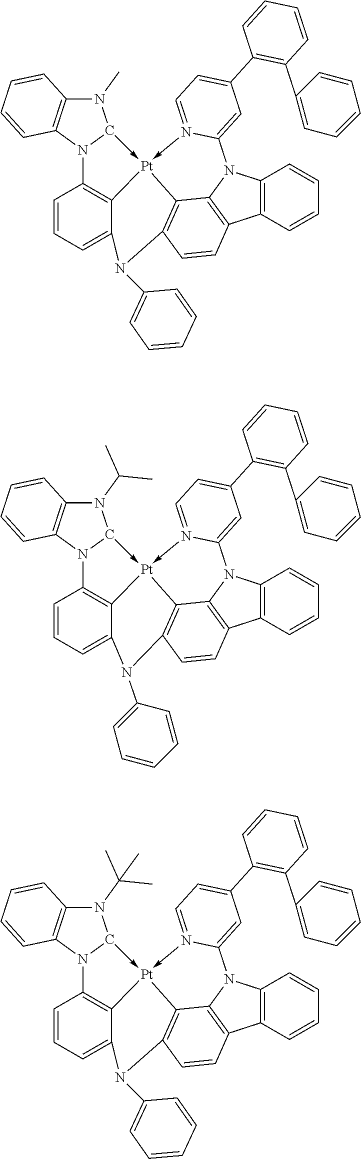

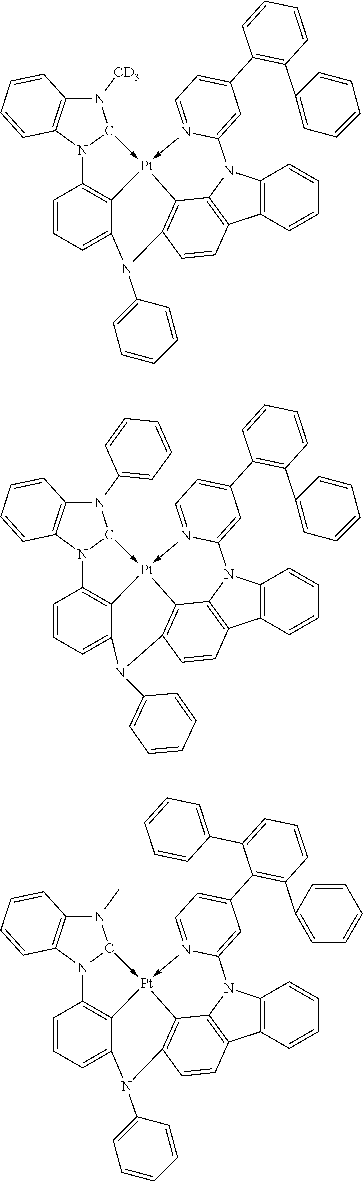

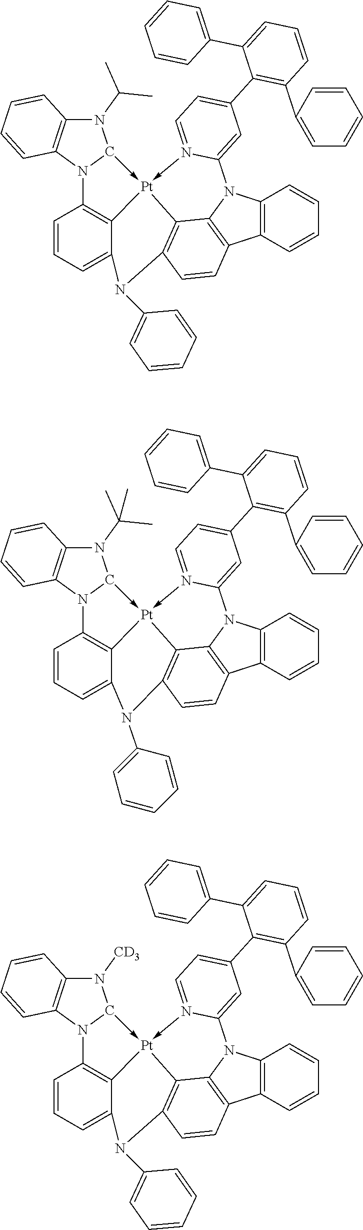

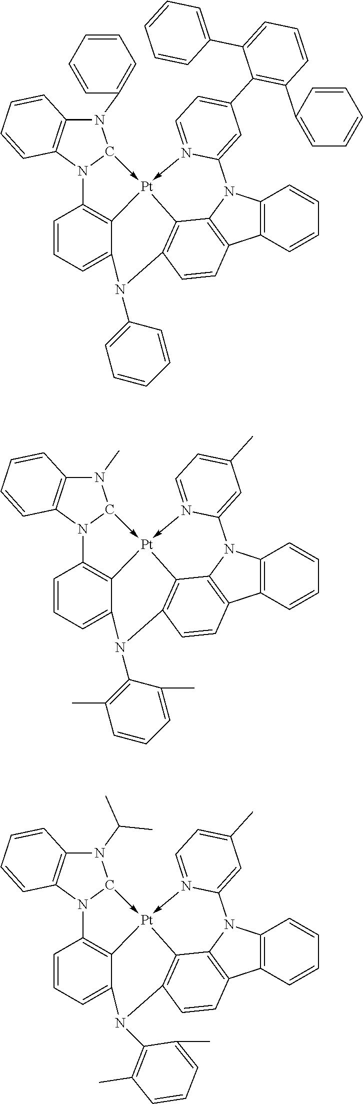

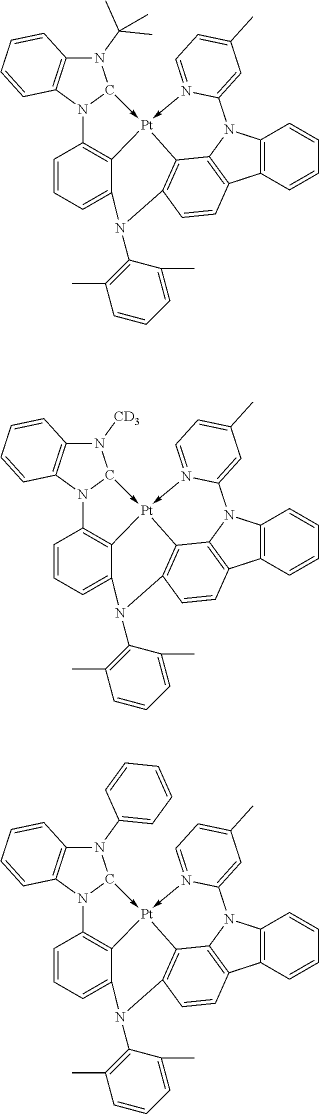

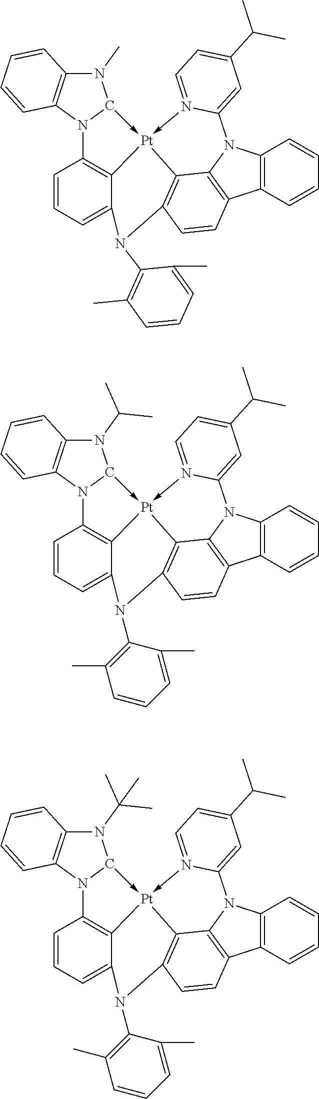

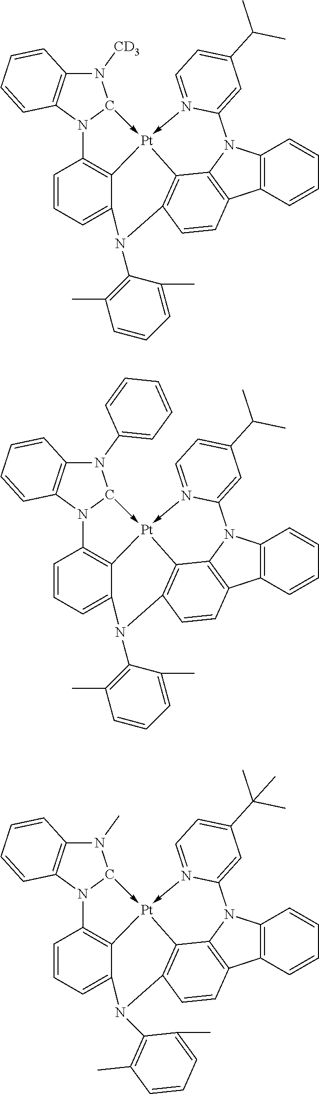

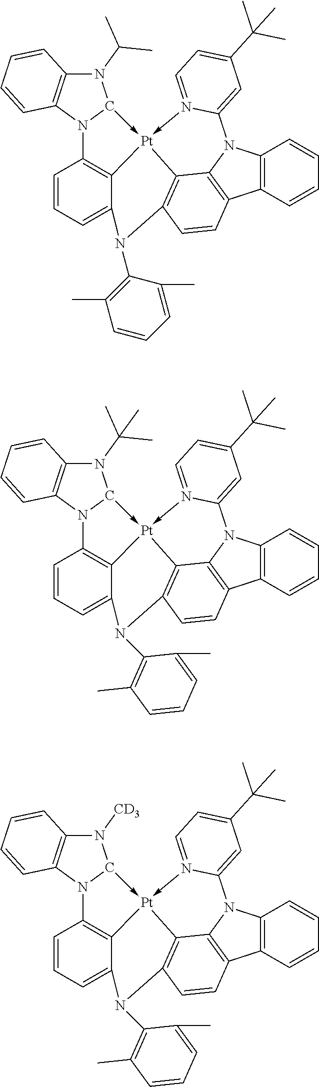

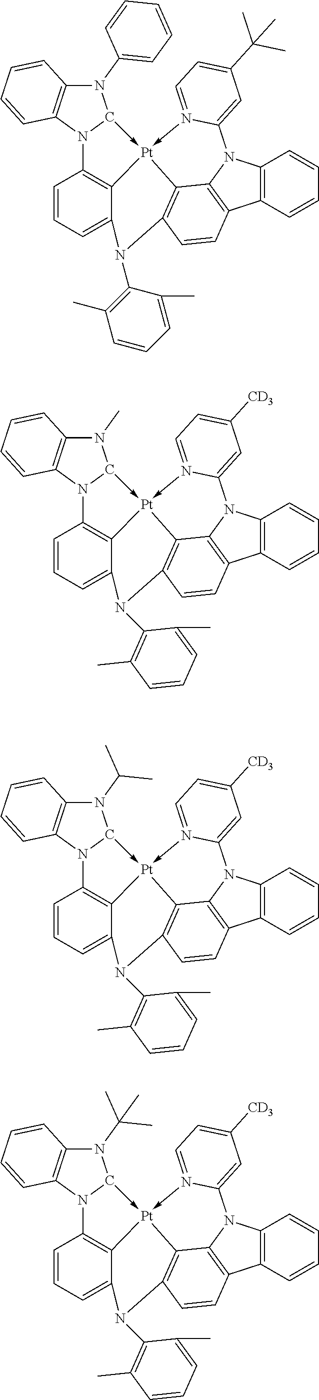

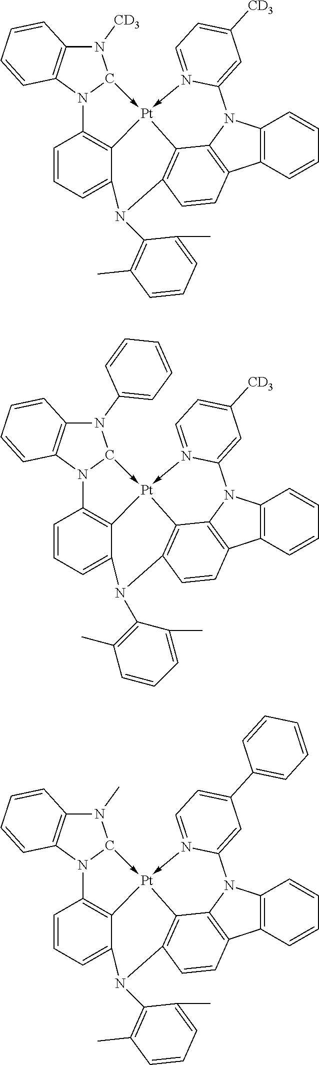

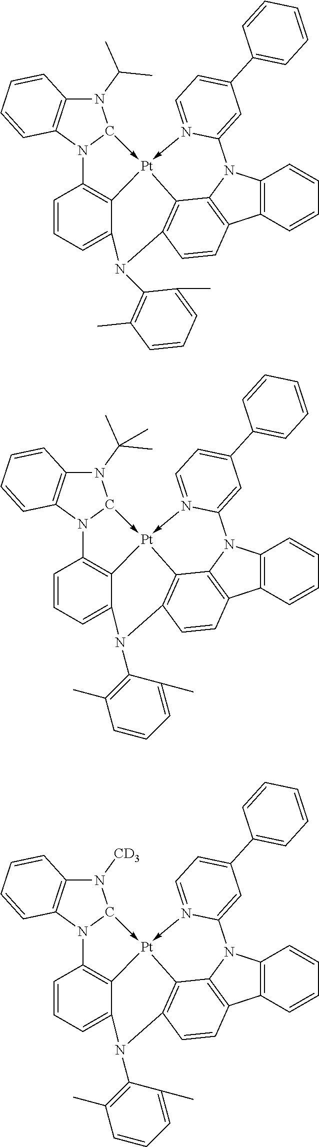

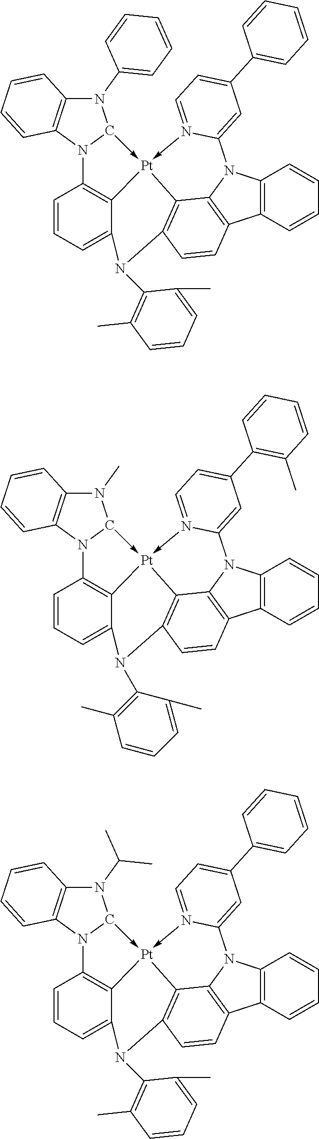

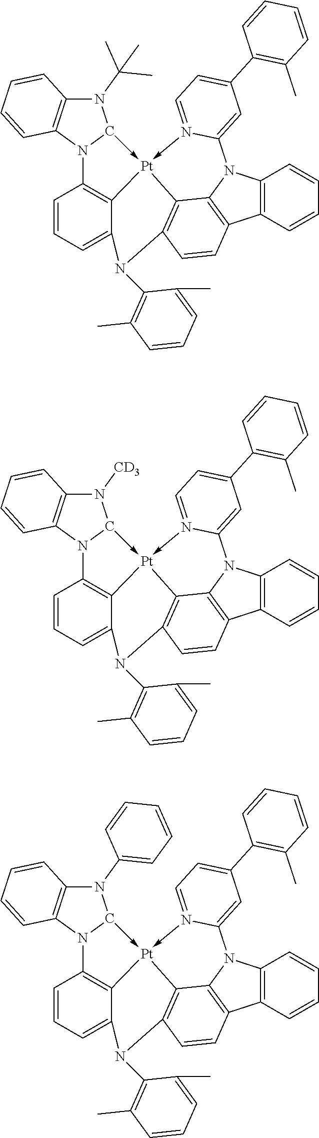

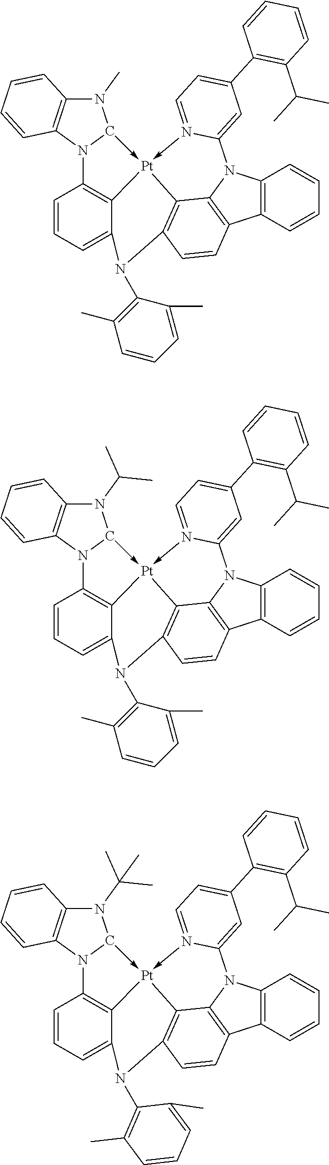

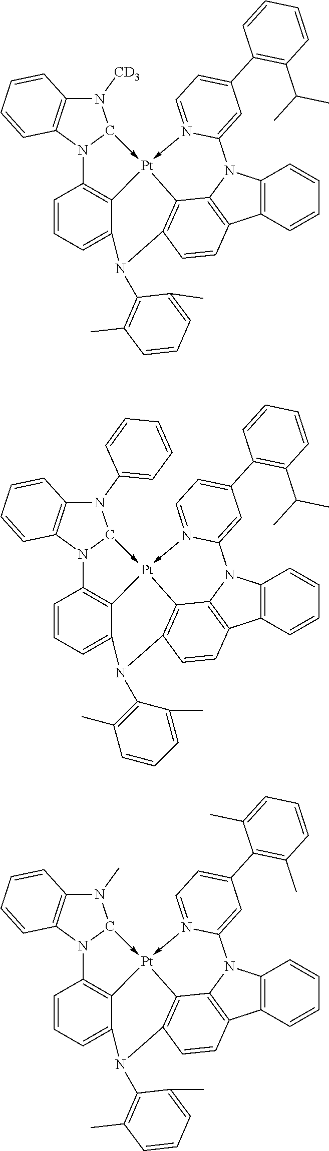

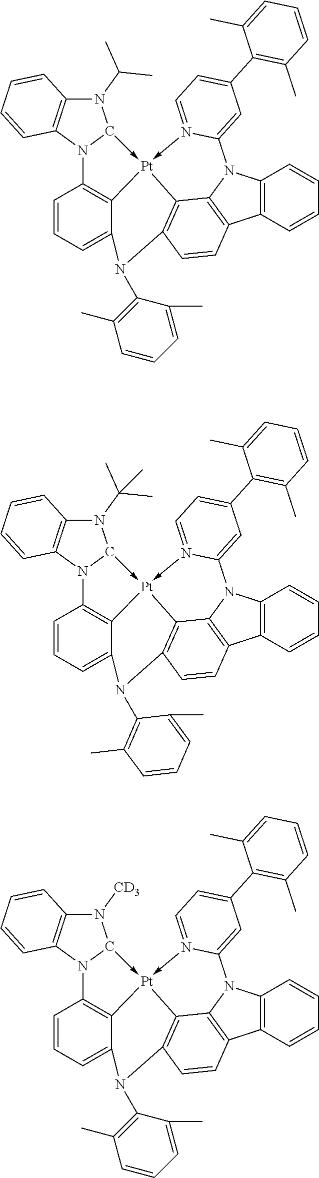

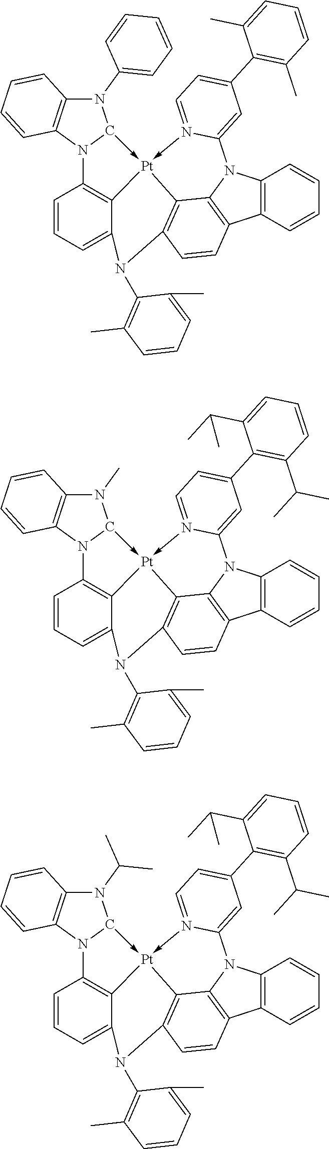

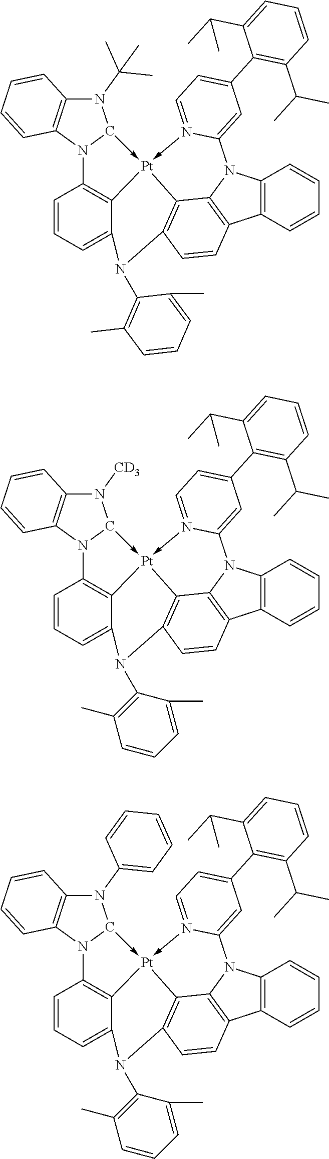

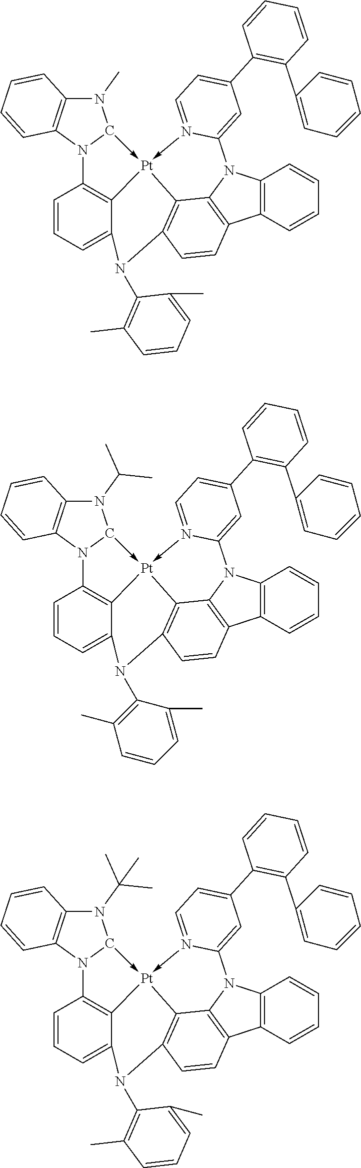

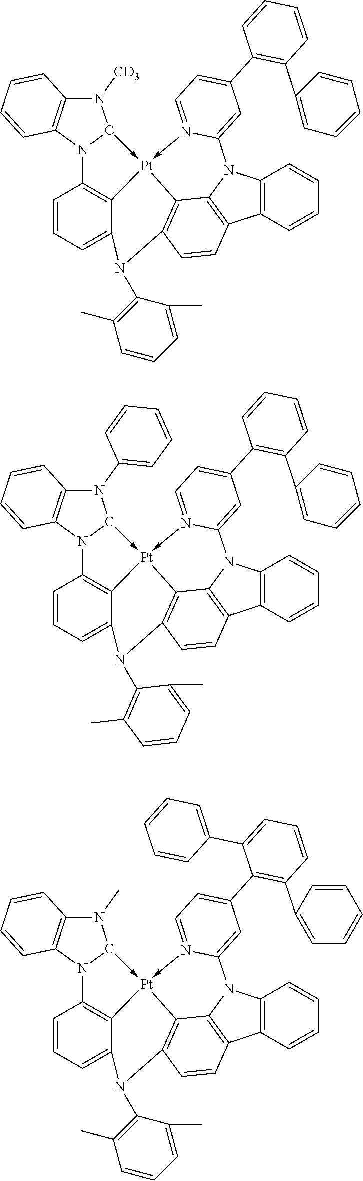

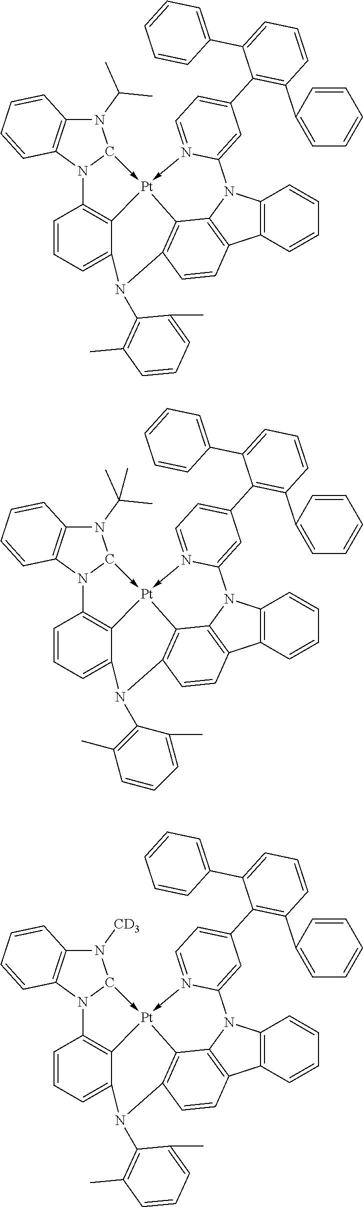

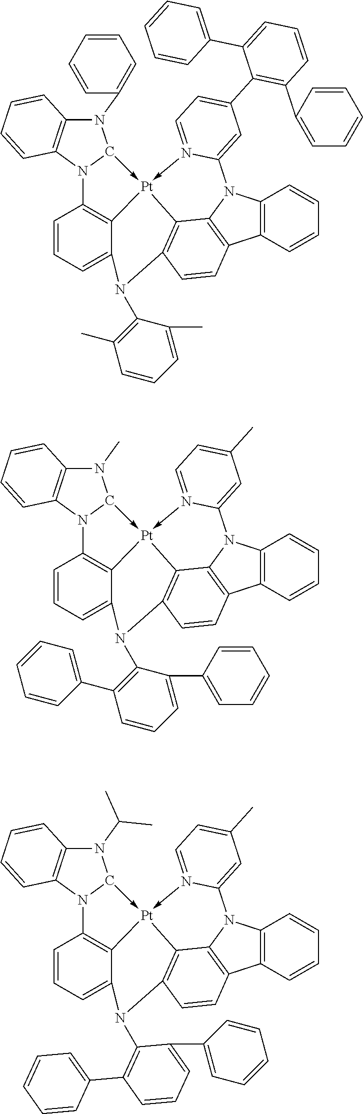

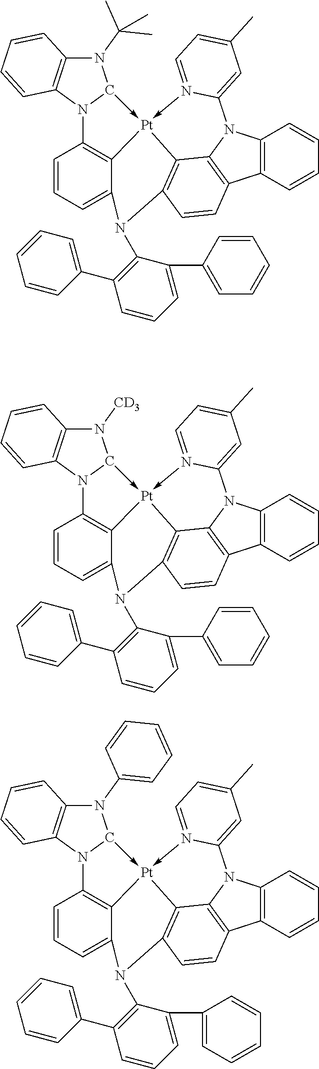

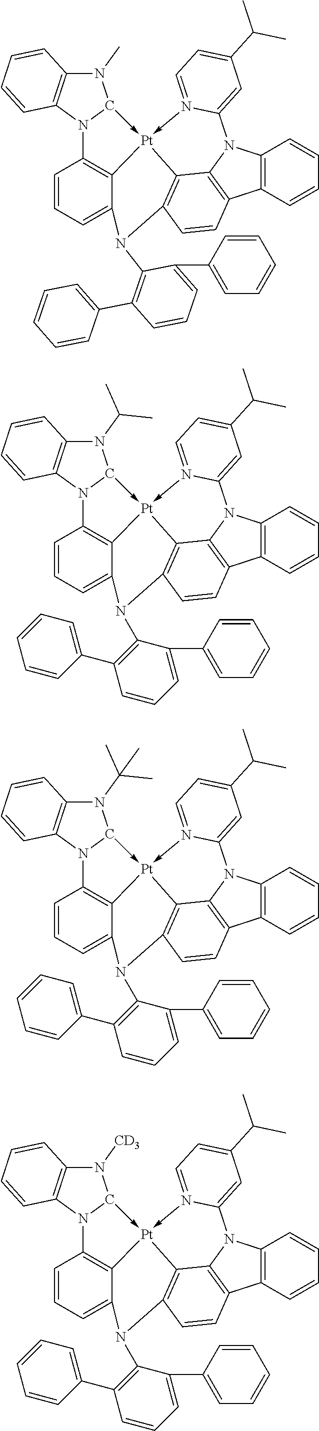

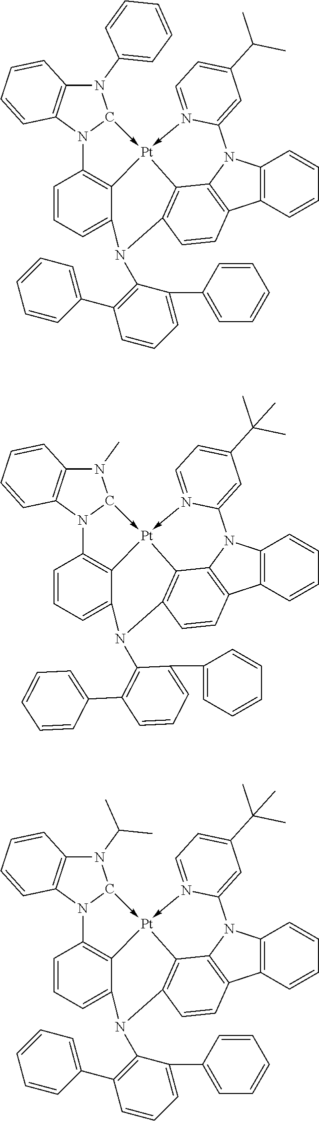

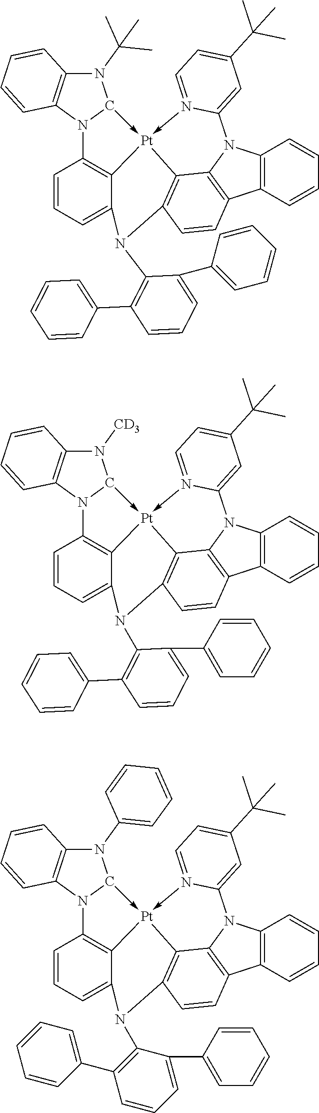

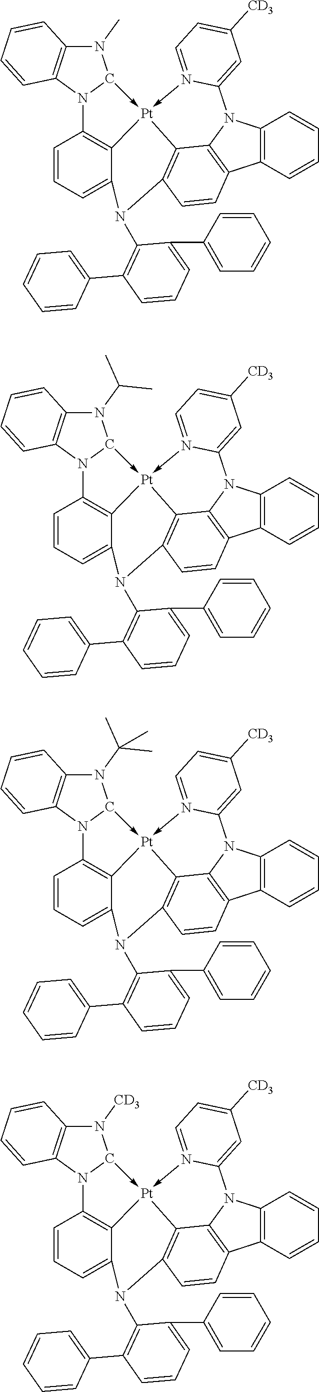

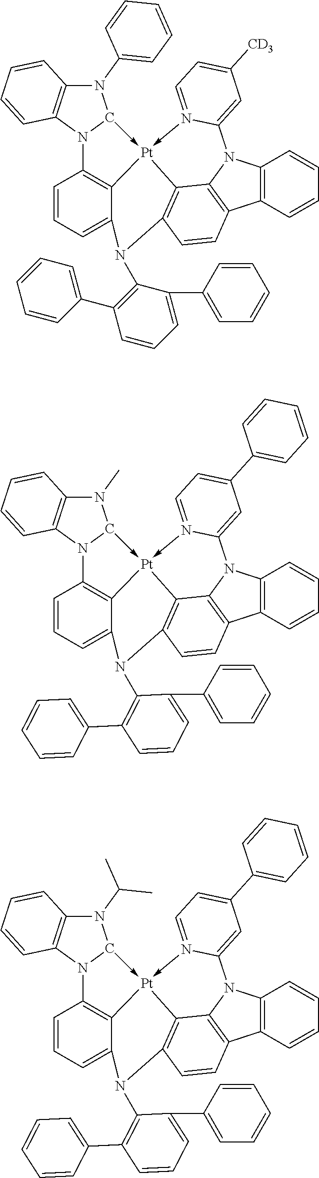

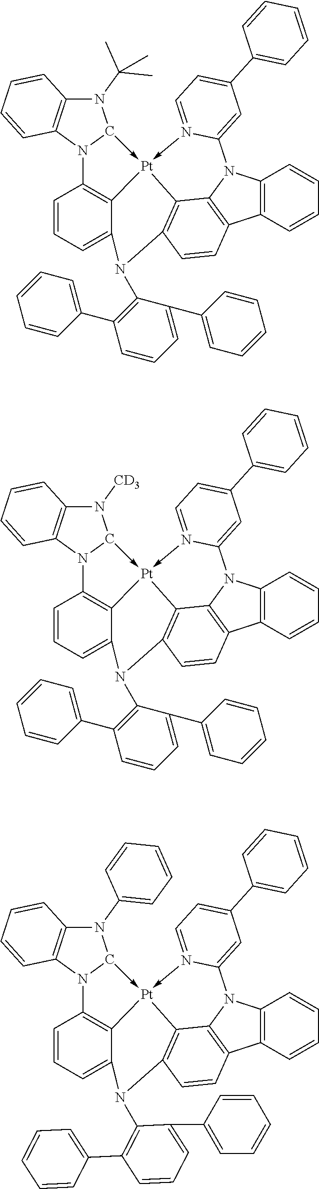

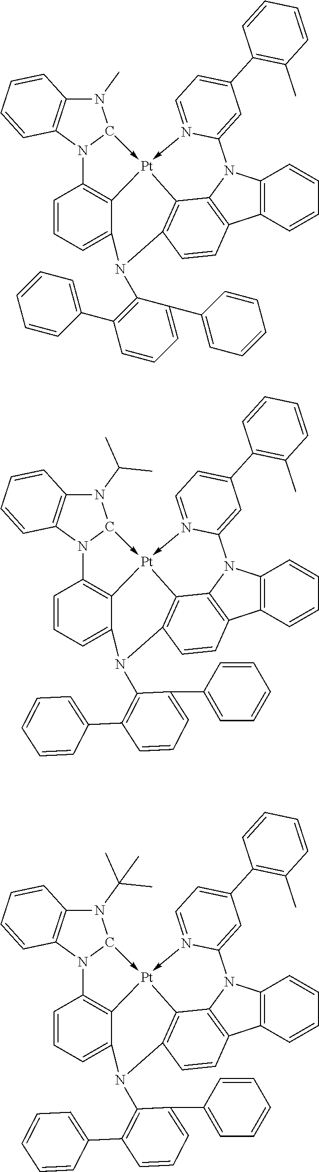

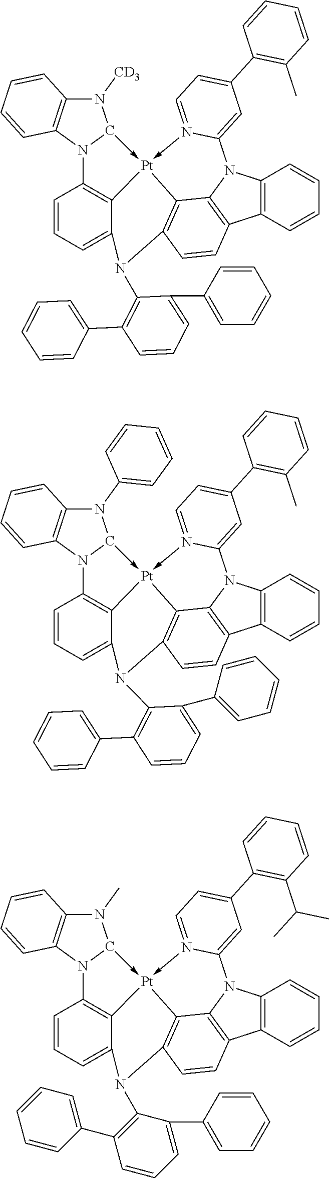

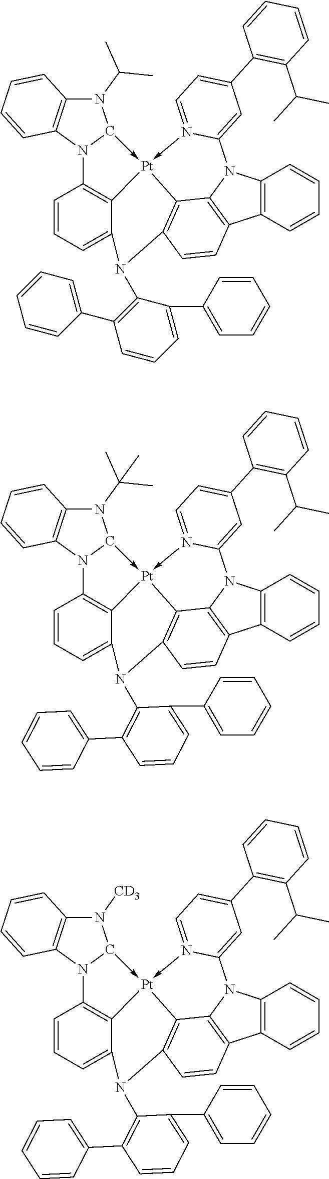

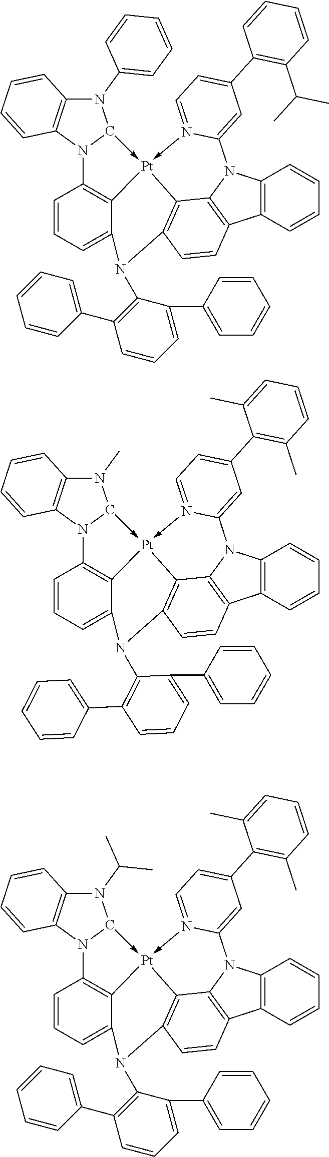

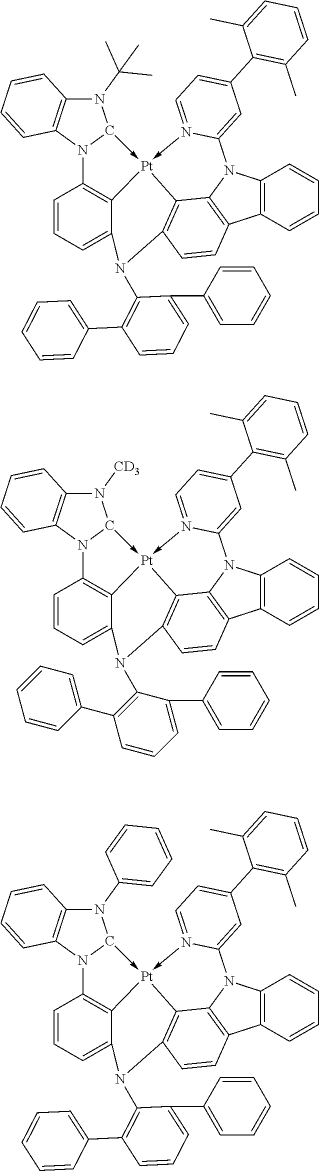

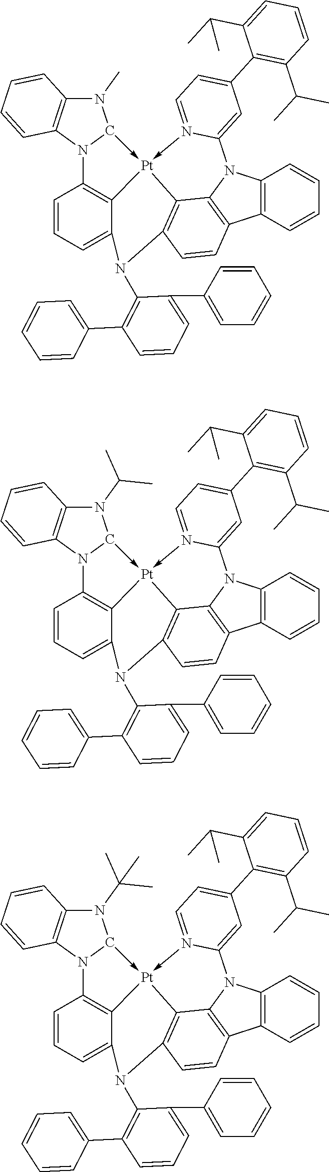

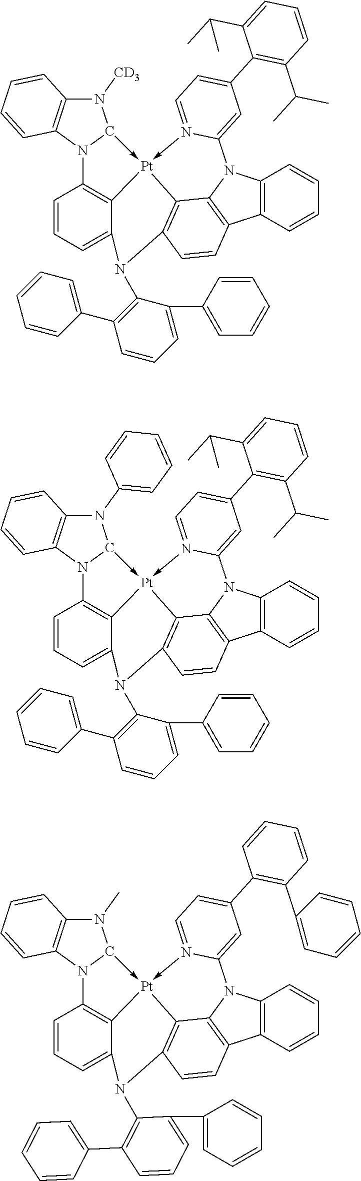

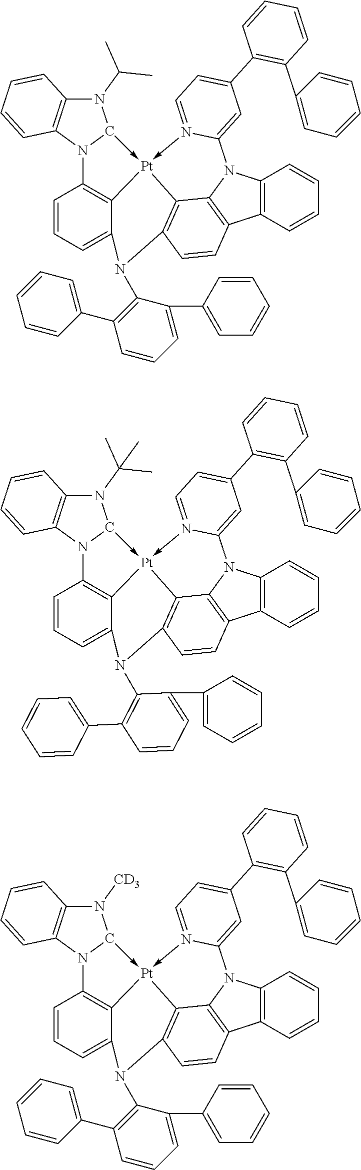

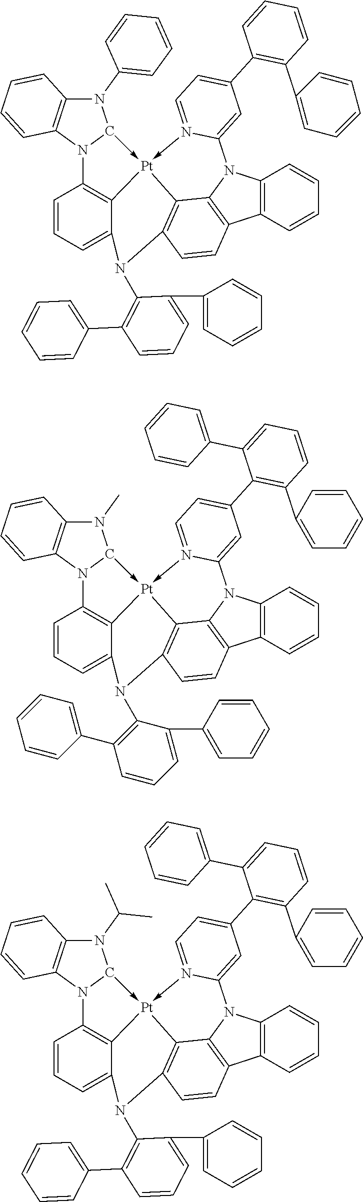

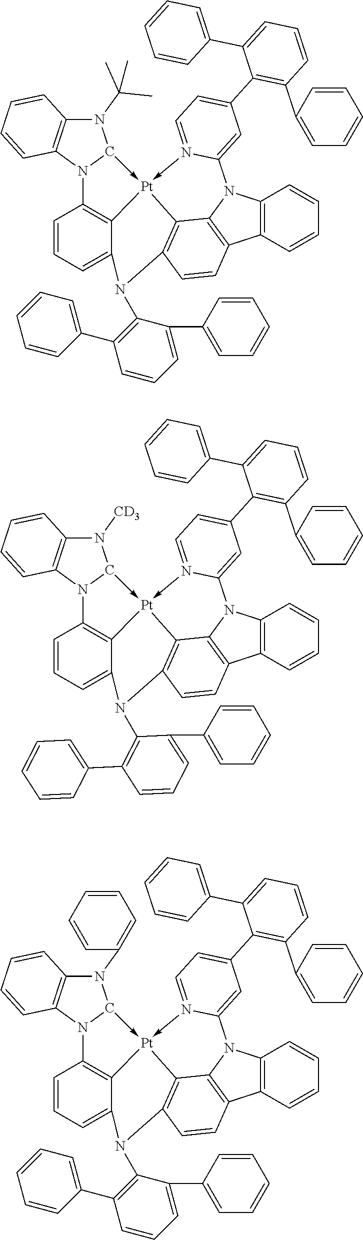

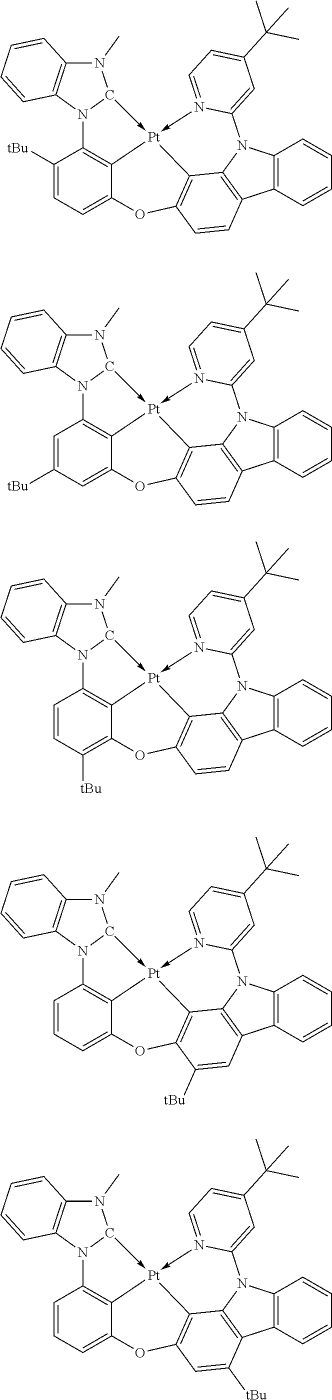

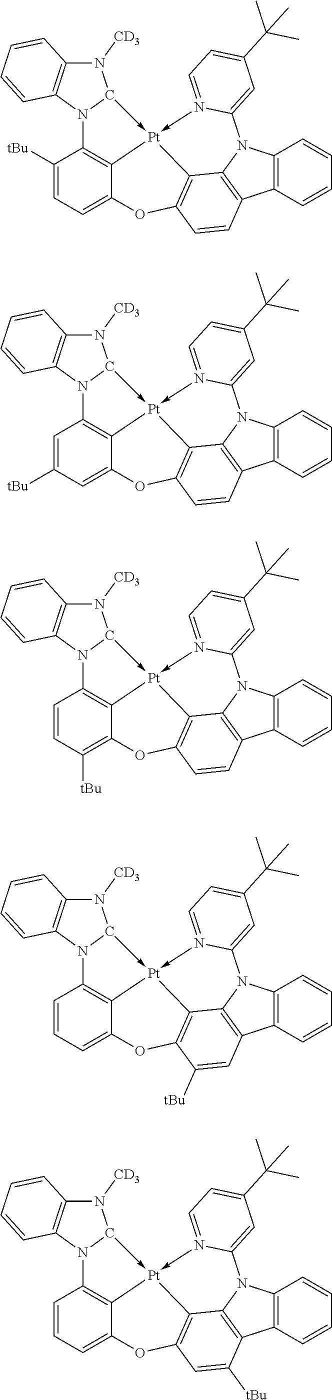

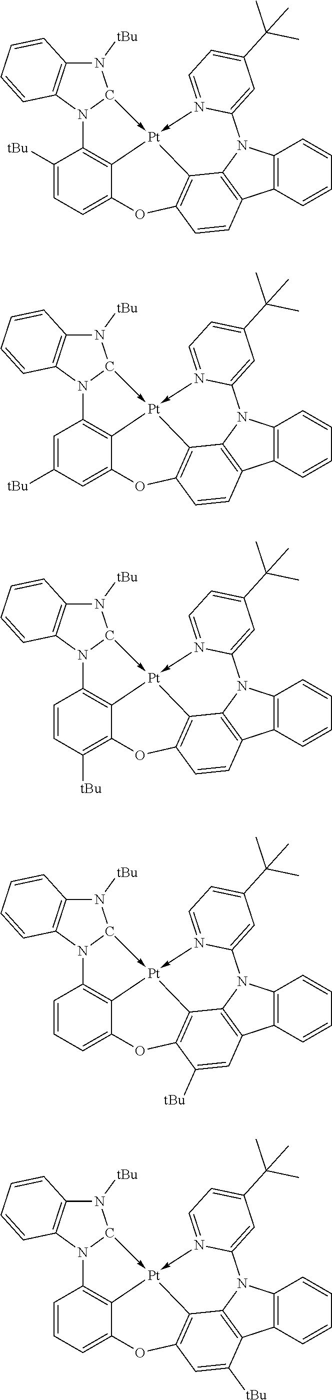

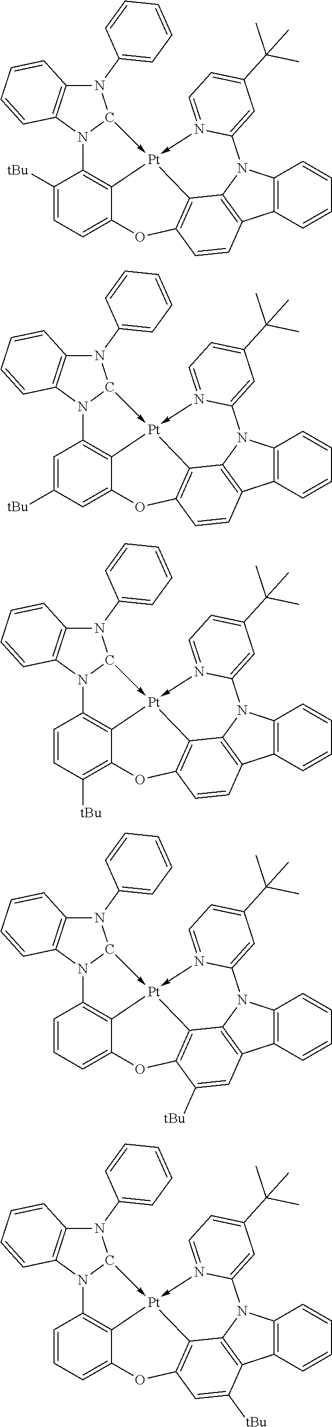

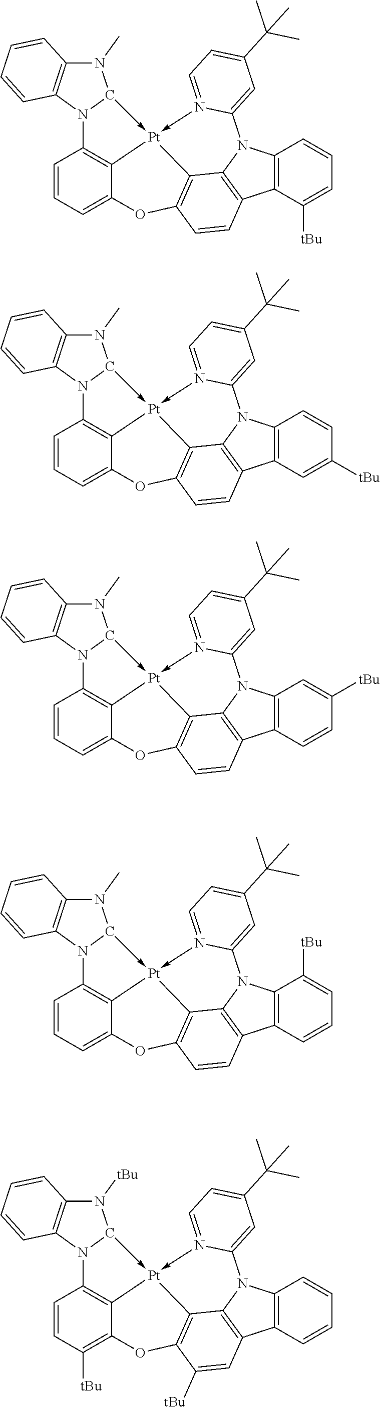

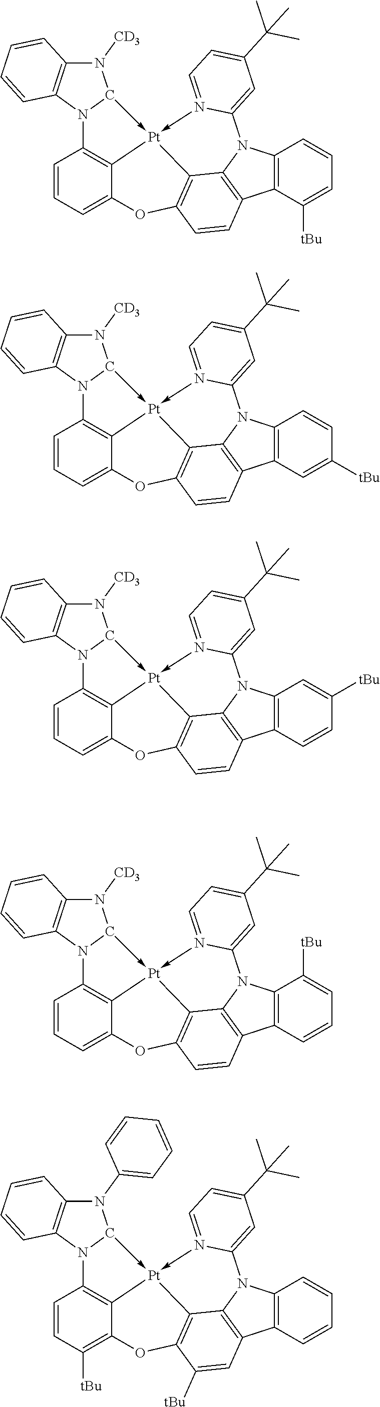

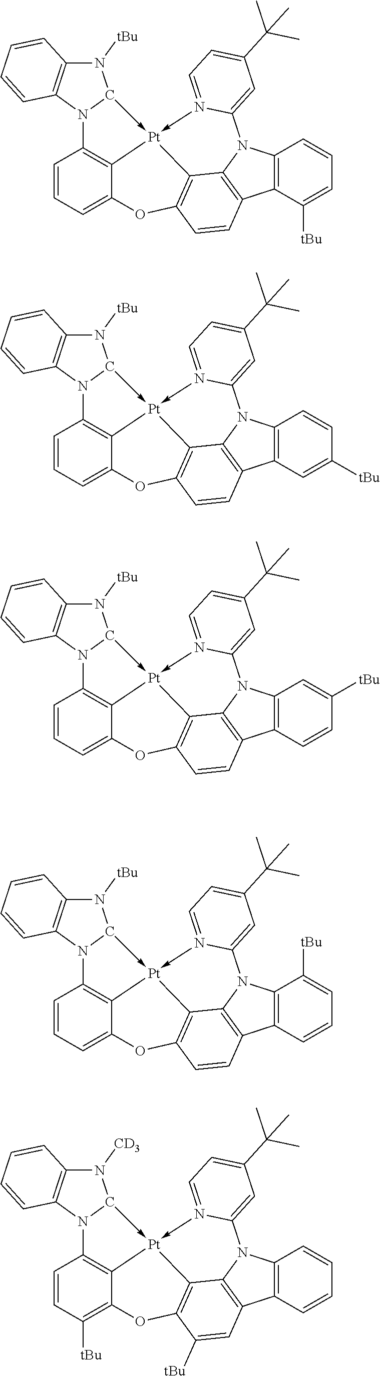

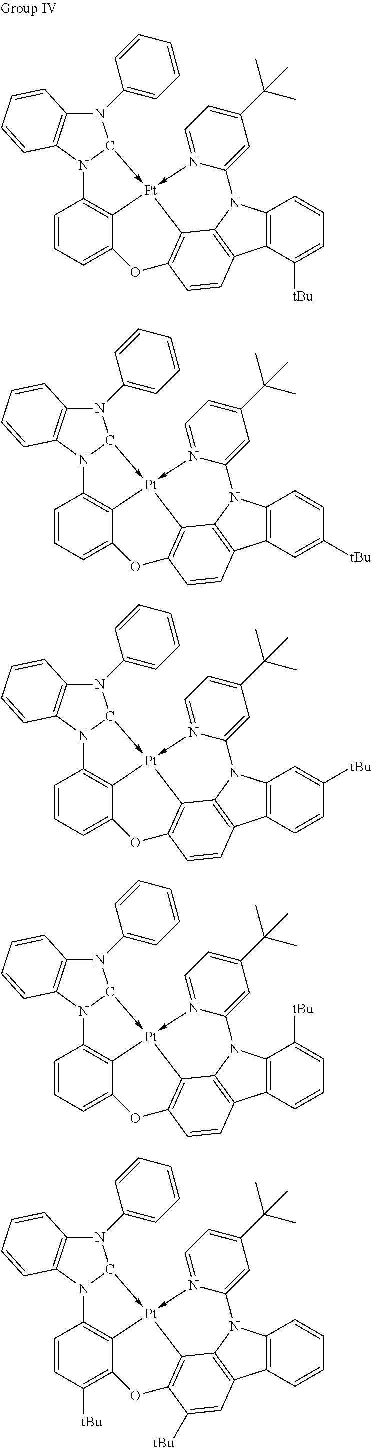

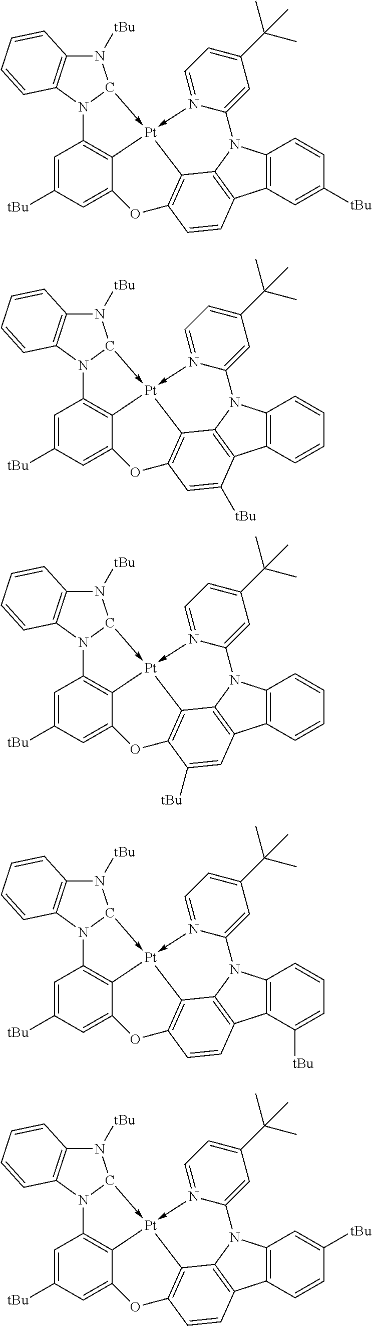

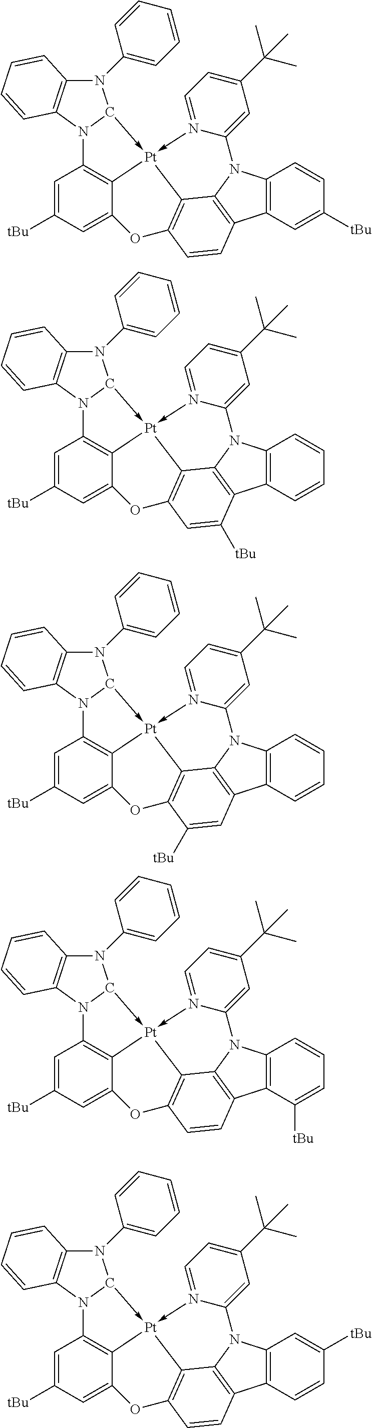

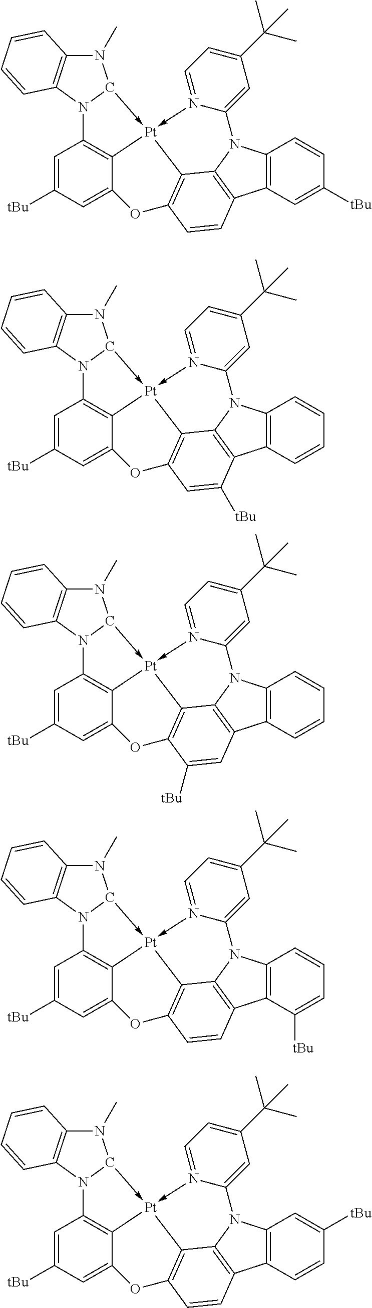

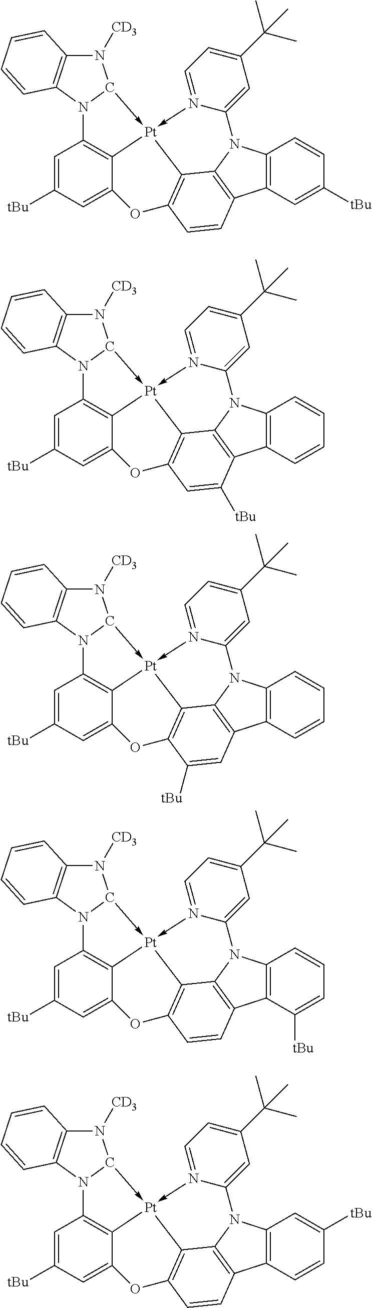

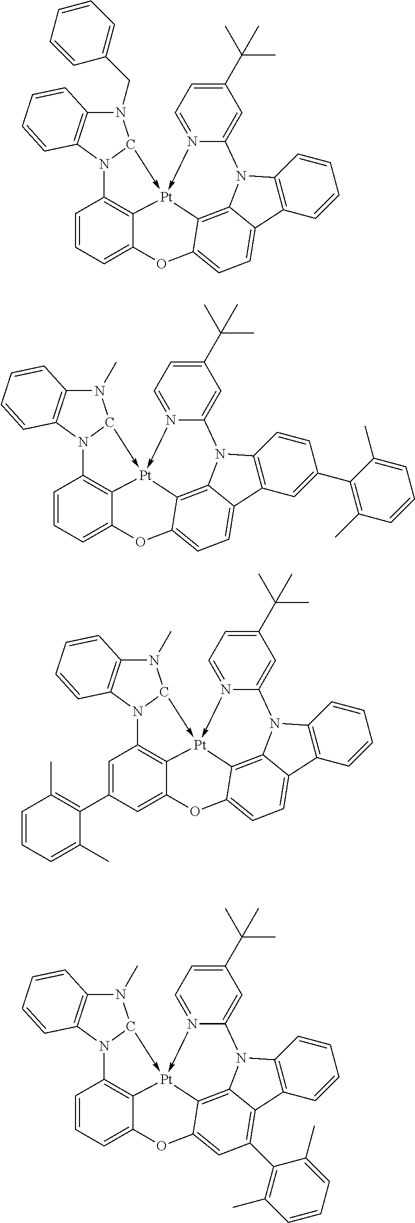

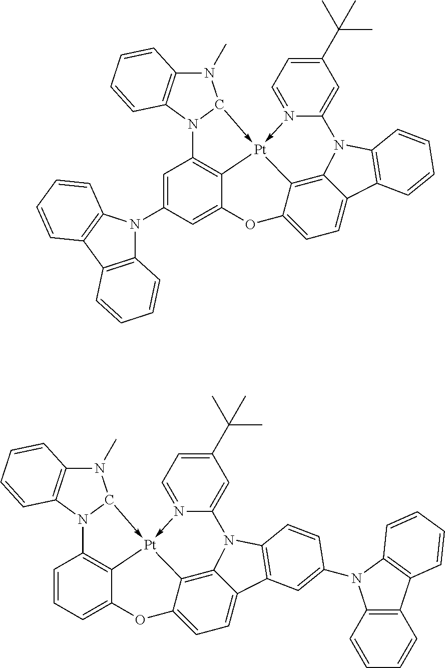

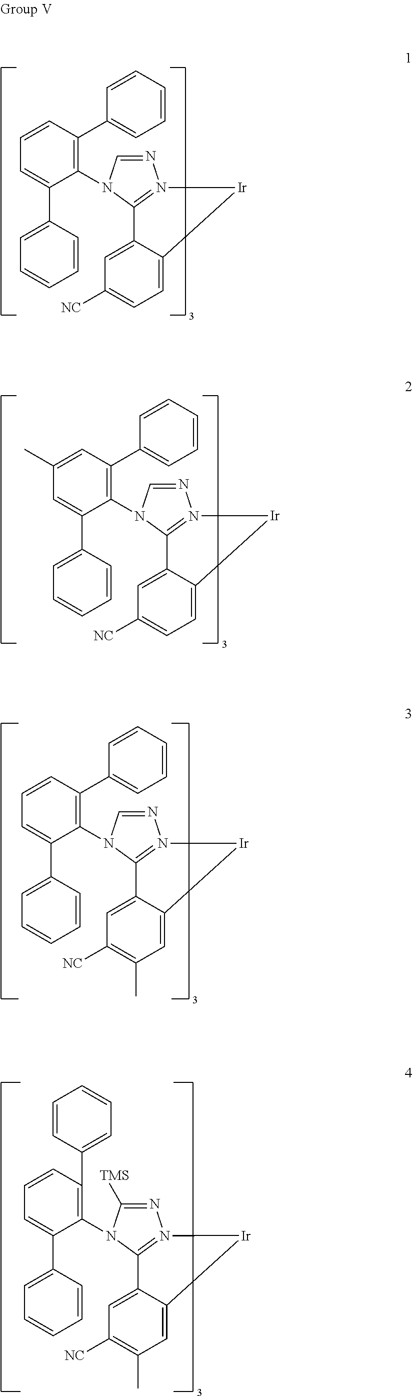

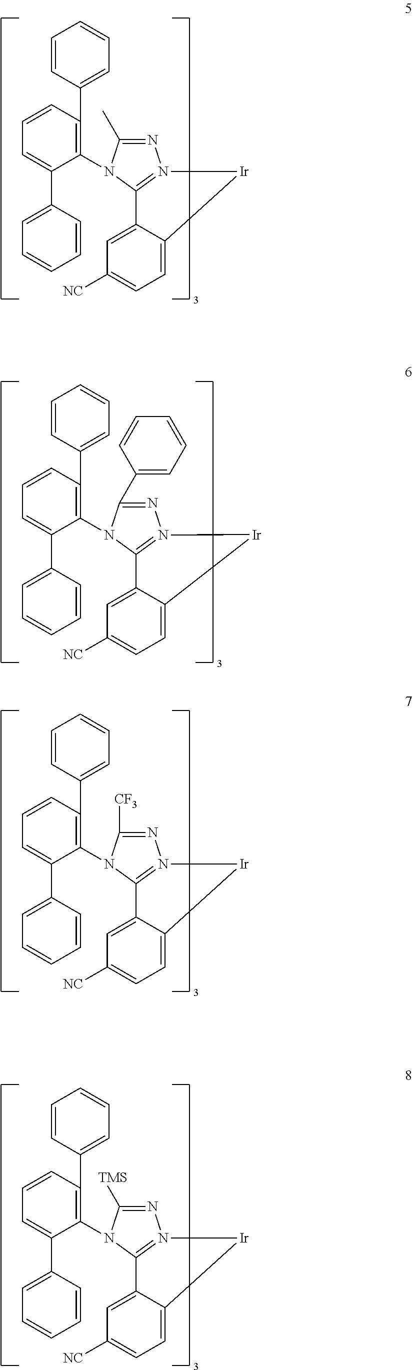

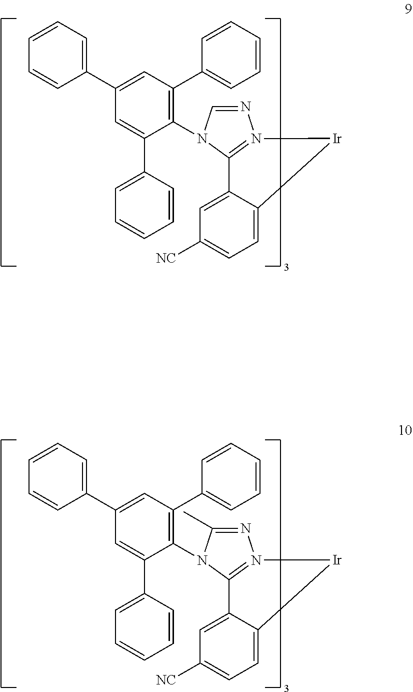

a compound represented by Formula A:

(L₁₀₁)ₙ₁₀₁-M₁₀₁-(L₁₀₂)ₘ₁₀₁ Formula A
wherein L₁₀₁, n₁₀₁, M₁₀₁, L₁₀₂, and m101 in Formula A may each be the same as described in Tables 1 to 3:

TABLE 1

| Compound | | | | | |
|---|---|---|---|---|---|
| name | L101 | n101 | M101 | L102 | m101 |
| BD001 | LM1 | 3 | Ir | — | 0 |
| BD002 | LM2 | 3 | Ir | — | 0 |
| BD003 | LM3 | 3 | Ir | — | 0 |
| BD004 | LM4 | 3 | Ir | — | 0 |
| BD005 | LM5 | 3 | Ir | — | 0 |
| BD006 | LM6 | 3 | Ir | — | 0 |
| BD007 | LM7 | 3 | Ir | — | 0 |
| BD008 | LM8 | 3 | Ir | — | 0 |
| BD009 | LM9 | 3 | Ir | — | 0 |
| BD010 | LM10 | 3 | Ir | — | 0 |
| BD011 | LM11 | 3 | Ir | — | 0 |
| BD012 | LM12 | 3 | Ir | — | 0 |
| BD013 | LM13 | 3 | Ir | — | 0 |
| BD014 | LM14 | 3 | Ir | — | 0 |
| BD015 | LM15 | 3 | Ir | — | 0 |
| BD016 | LM16 | 3 | Ir | — | 0 |
| BD017 | LM17 | 3 | Ir | — | 0 |
| BD018 | LM18 | 3 | Ir | — | 0 |
| BD019 | LM19 | 3 | Ir | — | 0 |
| BD020 | LM20 | 3 | Ir | — | 0 |
| BD021 | LM21 | 3 | Ir | — | 0 |
| BD022 | LM22 | 3 | Ir | — | 0 |
| BD023 | LM23 | 3 | Ir | — | 0 |
| BD024 | LM24 | 3 | Ir | — | 0 |
| BD025 | LM25 | 3 | Ir | — | 0 |
| BD026 | LM26 | 3 | Ir | — | 0 |
| BD027 | LM27 | 3 | Ir | — | 0 |
| BD028 | LM28 | 3 | Ir | — | 0 |
| BD029 | LM29 | 3 | Ir | — | 0 |
| BD030 | LM30 | 3 | Ir | — | 0 |
| BD031 | LM31 | 3 | Ir | — | 0 |
| BD032 | LM32 | 3 | Ir | — | 0 |
| BD033 | LM33 | 3 | Ir | — | 0 |
| BD034 | LM34 | 3 | Ir | — | 0 |
| BD035 | LM35 | 3 | Ir | — | 0 |
| BD036 | LM36 | 3 | Ir | — | 0 |
| BD037 | LM37 | 3 | Ir | — | 0 |
| BD038 | LM38 | 3 | Ir | — | 0 |
| BD039 | LM39 | 3 | In | — | 0 |
| BD040 | LM40 | 3 | Ir | — | 0 |
| BD041 | LM41 | 3 | Ir | — | 0 |
| BD042 | LM42 | 3 | Ir | — | 0 |
| BD043 | LM43 | 3 | Ir | — | 0 |
| BD044 | LM44 | 3 | Ir | — | 0 |
| BD045 | LM45 | 3 | Ir | — | 0 |
| BD046 | LM46 | 3 | Ir | — | 0 |
| BD047 | LM47 | 3 | Ir | — | 0 |
| BD048 | LM48 | 3 | Ir | — | 0 |
| BD049 | LM49 | 3 | Ir | — | 0 |
| BD050 | LM50 | 3 | Ir | — | 0 |
| BD051 | LM51 | 3 | Ir | — | 0 |
| BD052 | LM52 | 3 | Ir | — | 0 |
| BD053 | LM53 | 3 | Ir | — | 0 |
| BD054 | LM54 | 3 | Ir | — | 0 |
| BD055 | LM55 | 3 | Ir | — | 0 |
| BD056 | LM56 | 3 | Ir | — | 0 |
| BD057 | LM57 | 3 | Ir | — | 0 |
| BD058 | LM58 | 3 | Ir | — | 0 |
| BD059 | LM59 | 3 | Ir | — | 0 |
| BD060 | LM60 | 3 | Ir | — | 0 |
| BD061 | LM61 | 3 | Ir | — | 0 |
| BD062 | LM62 | 3 | Ir | — | 0 |
| BD063 | LM63 | 3 | Ir | — | 0 |
| BD064 | LM64 | 3 | Ir | — | 0 |
| BD065 | LM65 | 3 | Ir | — | 0 |
| BD066 | LM66 | 3 | Ir | — | 0 |
| BD067 | LM67 | 3 | Ir | — | 0 |
| BD068 | LM68 | 3 | Ir | — | 0 |
| BD069 | LM69 | 3 | Ir | — | 0 |
| BD070 | LM70 | 3 | Ir | — | 0 |
| BD071 | LM71 | 3 | Ir | — | 0 |
| BD072 | LM72 | 3 | Ir | — | 0 |
| BD073 | LM73 | 3 | Ir | — | 0 |
| BD074 | LM74 | 3 | Ir | — | 0 |
| BD075 | LM75 | 3 | Ir | — | 0 |
| BD076 | LM76 | 3 | Ir | — | 0 |
| BD077 | LM77 | 3 | Ir | — | 0 |
| BD078 | LM78 | 3 | Ir | — | 0 |
| BD079 | LM79 | 3 | Ir | — | 0 |
| BD080 | LM80 | 3 | Ir | — | 0 |
| BD081 | LM81 | 3 | Ir | — | 0 |
| BD082 | LM82 | 3 | Ir | — | 0 |
| BD083 | LM83 | 3 | Ir | — | 0 |
| BD084 | LM84 | 3 | Ir | — | 0 |
| BD085 | LM85 | 3 | Ir | — | 0 |
| BD086 | LM86 | 3 | Ir | — | 0 |
| BD087 | LM87 | 3 | Ir | — | 0 |
| BD088 | LM88 | 3 | Ir | — | 0 |
| BD089 | LM89 | 3 | Ir | — | 0 |
| BD090 | LM90 | 3 | Ir | — | 0 |
| BD091 | LM91 | 3 | Ir | — | 0 |
| BD092 | LM92 | 3 | Ir | — | 0 |
| BD093 | LM93 | 3 | Ir | — | 0 |
| BD094 | LM94 | 3 | Ir | — | 0 |
| BD095 | LM95 | 3 | Ir | — | 0 |
| BD096 | LM96 | 3 | Ir | — | 0 |
| BD097 | LM97 | 3 | Ir | — | 0 |
| BD098 | LM98 | 3 | Ir | — | 0 |
| BD099 | LM99 | 3 | Ir | — | 0 |
| BD100 | LM100 | 3 | Ir | — | 0 |

TABLE 2

| Compound | | | | | |
|---|---|---|---|---|---|
| name | L101 | n101 | M101 | L102 | m101 |
| BD101 | LM101 | 3 | Ir | — | 0 |
| BD102 | LM102 | 3 | Ir | — | 0 |
| BD103 | LM103 | 3 | Ir | — | 0 |
| BD104 | LM104 | 3 | Ir | — | 0 |
| BD105 | LM105 | 3 | Ir | — | 0 |
| BD106 | LM106 | 3 | Ir | — | 0 |
| BD107 | LM107 | 3 | Ir | — | 0 |
| BD108 | LM108 | 3 | Ir | — | 0 |
| BD109 | LM109 | 3 | Ir | — | 0 |
| BD110 | LM110 | 3 | Ir | — | 0 |
| BD111 | LM111 | 3 | Ir | — | 0 |
| BD112 | LM112 | 3 | Ir | — | 0 |
| BD113 | LM113 | 3 | Ir | — | 0 |
| BD114 | LM114 | 3 | Ir | — | 0 |
| BD115 | LM115 | 3 | Ir | — | 0 |
| BD116 | LM116 | 3 | Ir | — | 0 |
| BD117 | LM117 | 3 | Ir | — | 0 |
| BD118 | LM118 | 3 | Ir | — | 0 |
| BD119 | LM119 | 3 | Ir | — | 0 |
| BD120 | LM120 | 3 | Ir | — | 0 |
| BD121 | LM121 | 3 | Ir | — | 0 |
| BD122 | LM122 | 3 | Ir | — | 0 |
| BD123 | LM123 | 3 | Ir | — | 0 |
| BD124 | LM124 | 3 | Ir | — | 0 |
| BD125 | LM125 | 3 | Ir | — | 0 |
| BD126 | LM126 | 3 | Ir | — | 0 |
| BD127 | LM127 | 3 | Ir | — | 0 |
| BD128 | LM128 | 3 | Ir | — | 0 |
| BD129 | LM129 | 3 | Ir | — | 0 |
| BD130 | LM130 | 3 | Ir | — | 0 |
| BD131 | LM131 | 3 | Ir | — | 0 |
| BD132 | LM132 | 3 | Ir | — | 0 |
| BD133 | LM133 | 3 | Ir | — | 0 |
| BD134 | LM134 | 3 | Ir | — | 0 |
| BD135 | LM135 | 3 | Ir | — | 0 |
| BD136 | LM136 | 3 | Ir | — | 0 |
| BD137 | LM137 | 3 | Ir | — | 0 |
| BD138 | LM138 | 3 | Ir | — | 0 |
| BD139 | LM139 | 3 | Ir | — | 0 |
| BD140 | LM140 | 3 | Ir | — | 0 |
| BD141 | LM141 | 3 | Ir | — | 0 |
| BD142 | LM142 | 3 | Ir | — | 0 |
| BD143 | LM143 | 3 | Ir | — | 0 |
| BD144 | LM144 | 3 | Ir | — | 0 |
| BD145 | LM145 | 3 | Ir | — | 0 |
| BD146 | LM146 | 3 | Ir | — | 0 |
| BD147 | LM147 | 3 | Ir | — | 0 |
| BD148 | LM148 | 3 | Ir | — | 0 |
| BD149 | LM149 | 3 | Ir | — | 0 |
| BD150 | LM150 | 3 | Ir | — | 0 |
| BD151 | LM151 | 3 | Ir | — | 0 |
| BD152 | LM152 | 3 | Ir | — | 0 |
| BD153 | LM153 | 3 | In | — | 0 |
| BD154 | LM154 | 3 | Ir | — | 0 |
| BD155 | LM155 | 3 | Ir | — | 0 |
| BD156 | LM156 | 3 | Ir | — | 0 |
| BD157 | LM157 | 3 | Ir | — | 0 |
| BD158 | LM158 | 3 | Ir | — | 0 |
| BD159 | LM159 | 3 | Ir | — | 0 |
| BD160 | LM160 | 3 | Ir | — | 0 |
| BD161 | LM161 | 3 | Ir | — | 0 |
| BD162 | LM162 | 3 | Ir | — | 0 |
| BD163 | LM163 | 3 | Ir | — | 0 |
| BD164 | LM164 | 3 | Ir | — | 0 |
| BD165 | LM165 | 3 | Ir | — | 0 |
| BD166 | LM166 | 3 | Ir | — | 0 |
| BD167 | LM167 | 3 | Ir | — | 0 |
| BD168 | LM168 | 3 | Ir | — | 0 |
| BD169 | LM169 | 3 | Ir | — | 0 |
| BD170 | LM170 | 3 | Ir | — | 0 |
| BD171 | LM171 | 3 | Ir | — | 0 |
| BD172 | LM172 | 3 | Ir | — | 0 |
| BD173 | LM173 | 3 | Ir | — | 0 |
| BD174 | LM174 | 3 | Ir | — | 0 |
| BD175 | LM175 | 3 | Ir | — | 0 |
| BD176 | LM176 | 3 | Ir | — | 0 |
| BD177 | LM177 | 3 | Ir | — | 0 |
| BD178 | LM178 | 3 | Ir | — | 0 |
| BD179 | LM179 | 3 | Ir | — | 0 |
| BD180 | LM180 | 3 | Ir | — | 0 |
| BD181 | LM181 | 3 | Ir | — | 0 |
| BD182 | LM182 | 3 | Ir | — | 0 |
| BD183 | LM183 | 3 | Ir | — | 0 |
| BD184 | LM184 | 3 | Ir | — | 0 |
| BD185 | LM185 | 3 | Ir | — | 0 |
| BD186 | LM186 | 3 | Ir | — | 0 |
| BD187 | LM187 | 3 | Ir | — | 0 |
| BD188 | LM188 | 3 | Ir | — | 0 |
| BD189 | LM189 | 3 | Ir | — | 0 |
| BD190 | LM190 | 3 | Ir | — | 0 |
| BD191 | LM191 | 3 | Ir | — | 0 |
| BD192 | LM192 | 3 | Ir | — | 0 |
| BD193 | LM193 | 3 | Ir | — | 0 |
| BD194 | LM194 | 3 | Ir | — | 0 |
| BD195 | LM195 | 3 | Ir | — | 0 |
| BD196 | LM196 | 3 | Ir | — | 0 |
| BD197 | LM197 | 3 | Ir | — | 0 |
| BD198 | LM198 | 3 | Ir | — | 0 |
| BD199 | LM199 | 3 | Ir | — | 0 |
| BD200 | LM200 | 3 | Ir | — | 0 |

TABLE 3

| Compound | | | | | |
|---|---|---|---|---|---|
| name | L101 | n101 | M101 | L102 | m101 |
| BD201 | LM201 | 3 | Ir | — | 0 |
| BD202 | LM202 | 3 | Ir | — | 0 |
| BD203 | LM203 | 3 | Ir | — | 0 |
| BD204 | LM204 | 3 | Ir | — | 0 |
| BD205 | LM205 | 3 | Ir | — | 0 |
| BD206 | LM206 | 3 | Ir | — | 0 |
| BD207 | LM207 | 3 | Ir | — | 0 |
| BD208 | LM208 | 3 | Ir | — | 0 |
| BD209 | LM209 | 3 | Ir | — | 0 |
| BD210 | LM210 | 3 | Ir | — | 0 |
| BD211 | LM211 | 3 | Ir | — | 0 |
| BD212 | LM212 | 3 | Ir | — | 0 |
| BD213 | LM213 | 3 | Ir | — | 0 |
| BD214 | LM214 | 3 | Ir | — | 0 |
| BD215 | LM215 | 3 | Ir | — | 0 |
| BD216 | LM216 | 3 | Ir | — | 0 |
| BD217 | LM217 | 3 | Ir | — | 0 |
| BD218 | LM218 | 3 | Ir | — | 0 |
| BD219 | LM219 | 3 | Ir | — | 0 |
| BD220 | LM220 | 3 | Ir | — | 0 |
| BD221 | LM221 | 3 | Ir | — | 0 |
| BD222 | LM222 | 3 | Ir | — | 0 |
| BD223 | LM223 | 3 | Ir | — | 0 |
| BD224 | LM224 | 3 | Ir | — | 0 |
| BD225 | LM225 | 3 | Ir | — | 0 |
| BD226 | LM226 | 3 | Ir | — | 0 |
| BD227 | LM227 | 3 | Ir | — | 0 |
| BD228 | LM228 | 3 | Ir | — | 0 |
| BD229 | LM229 | 3 | Ir | — | 0 |
| BD230 | LM230 | 3 | Ir | — | 0 |
| BD231 | LM231 | 3 | Ir | — | 0 |
| BD232 | LM232 | 3 | Ir | — | 0 |
| BD233 | LM233 | 3 | Ir | — | 0 |
| BD234 | LM234 | 3 | Ir | — | 0 |
| BD235 | LM235 | 3 | Ir | — | 0 |
| BD236 | LM236 | 3 | Ir | — | 0 |
| BD237 | LM237 | 3 | Ir | — | 0 |
| BD238 | LM238 | 3 | Ir | — | 0 |
| BD239 | LM239 | 3 | Ir | — | 0 |
| BD240 | LM240 | 3 | Ir | — | 0 |
| BD241 | LM241 | 3 | Ir | — | 0 |
| BD242 | LM242 | 3 | Ir | — | 0 |
| BD243 | LM243 | 3 | Ir | — | 0 |
| BD244 | LFM1 | 3 | Ir | — | 0 |
| BD245 | LFM2 | 3 | Ir | — | 0 |
| BD246 | LFM3 | 3 | Ir | — | 0 |
| BD247 | LFM4 | 3 | Ir | — | 0 |
| BD248 | LFM5 | 3 | Ir | — | 0 |
| BD249 | LFM6 | 3 | Ir | — | 0 |
| BD250 | LFM7 | 3 | Ir | — | 0 |
| BD251 | LFP1 | 3 | Ir | — | 0 |
| BD252 | LFP2 | 3 | Ir | — | 0 |
| BD253 | LFP3 | 3 | Ir | — | 0 |
| BD254 | LFP4 | 3 | Ir | — | 0 |
| BD255 | LFP5 | 3 | Ir | — | 0 |
| BD256 | LFP6 | 3 | Ir | — | 0 |
| BD257 | LFP7 | 3 | Ir | — | 0 |
| BD258 | LM47 | 2 | Ir | AN1 | 1 |
| BD259 | LM47 | 2 | Ir | AN2 | 1 |
| BD260 | LM47 | 2 | Ir | AN3 | 1 |
| BD261 | LM47 | 2 | Ir | AN4 | 1 |
| BD262 | LM47 | 2 | Ir | AN5 | 1 |
| BD263 | LM11 | 2 | Pt | — | 0 |
| BD264 | LM13 | 2 | Pt | — | 0 |
| BD265 | LM15 | 2 | Pt | — | 0 |
| BD266 | LM45 | 2 | Pt | — | 0 |
| BD267 | LM47 | 2 | Pt | — | 0 |
| BD268 | LM49 | 2 | Pt | — | 0 |
| BD269 | LM98 | 2 | Pt | — | 0 |
| BD270 | LM100 | 2 | Pt | — | 0 |
| BD271 | LM102 | 2 | Pt | — | 0 |
| BD272 | LM132 | 2 | Pt | — | 0 |
| BD273 | LM134 | 2 | Pt | — | 0 |
| BD274 | LM136 | 2 | Pt | — | 0 |
| BD275 | LM151 | 2 | Pt | — | 0 |
| BD276 | LM153 | 2 | Pt | — | 0 |
| BD277 | LM158 | 2 | Pt | — | 0 |
| BD278 | LM180 | 2 | Pt | — | 0 |
| BD279 | LM182 | 2 | Pt | — | 0 |
| BD280 | LM187 | 2 | Pt | — | 0 |
| BD281 | LM201 | 2 | Pt | — | 0 |
| BD282 | LM206 | 2 | Pt | — | 0 |
| BD283 | LM211 | 2 | Pt | — | 0 |
| BD284 | LM233 | 2 | Pt | — | 0 |
| BD285 | LM235 | 2 | Pt | — | 0 |
| BD286 | LM240 | 2 | Pt | — | 0 |
| BD287 | LFM5 | 2 | Pt | — | 0 |
| BD288 | LFM6 | 2 | Pt | — | 0 |
| BD289 | LFM7 | 2 | Pt | — | 0 |
| BD290 | LFP5 | 2 | Pt | — | 0 |
| BD291 | LFP6 | 2 | Pt | — | 0 |
| BD292 | LFP7 | 2 | Pt | — | 0 |
| BD293 | LM47 | 1 | Pt | AN1 | 1 |
| BD294 | LM47 | 1 | Pt | AN2 | 1 |
| BD295 | LM47 | 1 | Pt | AN3 | 1 |
| BD296 | LM47 | 1 | Pt | AN4 | 1 |
| BD297 | LM47 | 1 | Pt | AN5 | 1 |

LM₁ to LM₂₄₃ in Tables 1 to 3 may each be understood by referring to Formulae 1-1 to 1-3 and Tables 4 to 6:

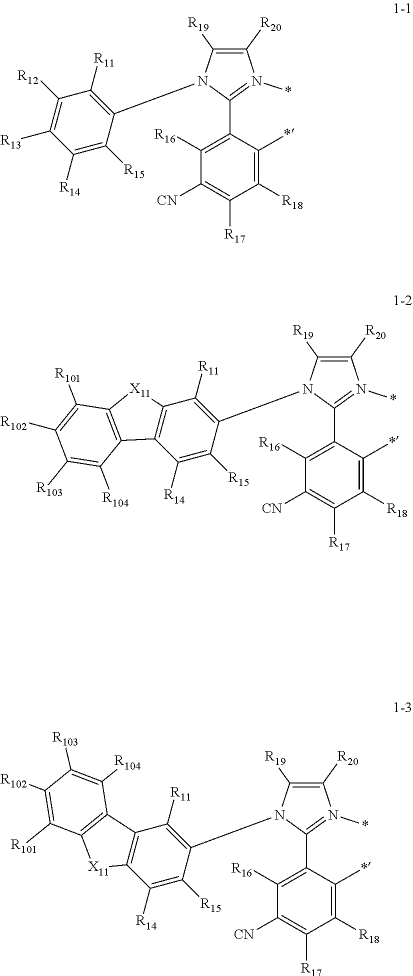

TABLE 4
Formula 1-1

| Ligand name | R11 | R12 | R13 | R14 | R15 | R16 | R17 | R18 | R19 | R20 |
|---|---|---|---|---|---|---|---|---|---|---|
| LM1 | X1 | H | X3 | H | X1 | H | H | H | H | D |
| LM2 | X1 | H | X3 | H | X1 | H | H | H | D | H |
| LM3 | X1 | H | X3 | H | X1 | H | H | H | D | D |
| LM4 | Y1 | H | X3 | H | Y1 | H | H | H | D | D |
| LM5 | Y2 | H | X3 | H | Y2 | H | H | H | D | D |
| LM6 | Y3 | H | X3 | H | Y3 | H | H | H | D | D |
| LM7 | Y3 | D | X3 | D | Y3 | H | H | H | D | D |
| LM8 | Y3 | D | X3 | D | Y3 | D | H | H | D | D |
| LM9 | Y3 | D | X3 | D | Y3 | D | D | H | D | D |
| LM10 | Y3 | D | X3 | D | Y3 | D | D | D | D | D |
| LM11 | Y3 | D | Y11 | D | Y3 | D | D | D | D | D |
| LM12 | Y3 | D | Y11 | D | Y3 | H | X1 | H | D | D |
| LM13 | Y3 | D | Y11 | D | Y3 | D | Y3 | D | D | D |
| LM14 | Y3 | D | Y11 | D | Y3 | H | X4 | H | D | D |
| LM15 | Y3 | D | Y11 | D | Y3 | D | Y12 | D | D | D |
| LM16 | X2 | H | X3 | H | X2 | H | H | H | H | D |
| LM17 | X2 | H | X3 | H | X2 | H | H | H | D | H |
| LM18 | X2 | H | X3 | H | X2 | H | H | H | D | D |
| LM19 | Y4 | H | X3 | H | Y4 | H | H | H | D | D |
| LM20 | Y5 | H | X3 | H | Y5 | H | H | H | D | D |
| LM21 | Y6 | H | X3 | H | Y6 | H | H | H | D | D |
| LM22 | Y7 | H | X3 | H | Y7 | H | H | H | D | D |
| LM23 | Y8 | H | X3 | H | Y8 | H | H | H | D | D |
| LM24 | Y9 | H | X3 | H | Y9 | H | H | H | D | D |
| LM25 | Y10 | H | X3 | H | Y10 | H | H | H | D | D |
| LM26 | Y10 | D | X3 | D | Y10 | H | H | H | D | D |
| LM27 | Y10 | D | X3 | D | Y10 | D | H | H | D | D |
| LM28 | Y10 | D | X3 | D | Y10 | D | D | H | D | D |
| LM29 | Y10 | D | X3 | D | Y10 | D | D | D | D | D |
| LM30 | Y10 | D | Y11 | D | Y10 | D | D | D | D | D |
| LM31 | Y10 | D | Y11 | D | Y10 | H | X1 | H | D | D |
| LM32 | Y10 | D | Y11 | D | Y10 | D | Y3 | D | D | D |
| LM33 | Y10 | D | Y11 | D | Y10 | H | X4 | H | D | D |
| LM34 | Y10 | D | Y11 | D | Y10 | D | Y12 | D | D | D |
| LM35 | X | H | X4 | H | X1 | H | H | H | H | D |
| LM36 | X1 | H | X4 | H | X1 | H | H | H | D | H |
| LM37 | X1 | H | X4 | H | X1 | H | H | H | D | D |
| LM38 | Y1 | H | X4 | H | Y1 | H | H | H | D | D |
| LM39 | Y2 | H | X4 | H | Y2 | H | H | H | D | D |
| LM40 | Y3 | H | X4 | H | Y3 | H | H | H | D | D |
| LM41 | Y3 | D | X4 | D | Y3 | H | H | H | D | D |
| LM42 | Y3 | D | X4 | D | Y3 | D | H | H | D | D |
| LM43 | Y3 | D | X4 | D | Y3 | D | D | H | D | D |
| LM44 | Y3 | D | X4 | D | Y3 | D | D | D | D | D |
| LM45 | Y3 | D | Y12 | D | Y3 | D | D | D | D | D |
| LM46 | Y3 | D | Y12 | D | Y3 | H | X1 | H | D | D |
| LM47 | Y3 | D | Y12 | D | Y3 | D | Y3 | D | D | D |
| LM48 | Y3 | D | Y12 | D | Y3 | H | X4 | H | D | D |
| LM49 | Y3 | D | Y12 | D | Y3 | D | Y12 | D | D | D |
| LM50 | X2 | H | X4 | H | X2 | H | H | H | H | D |
| LM51 | X2 | H | X4 | H | X2 | H | H | H | D | H |
| LM52 | X2 | H | X4 | H | X2 | H | H | H | D | D |
| LM53 | Y4 | H | X4 | H | Y4 | H | H | H | D | D |
| LM54 | Y5 | H | X4 | H | Y5 | H | H | H | D | D |
| LM55 | Y6 | H | X4 | H | Y6 | H | H | H | D | D |
| LM56 | Y7 | H | X4 | H | Y7 | H | H | H | D | D |
| LM57 | Y8 | H | X4 | H | Y8 | H | H | H | D | D |
| LM58 | Y9 | H | X4 | H | Y9 | H | H | H | D | D |
| LM59 | Y10 | H | X4 | H | Y10 | H | H | H | D | D |
| LM60 | Y10 | D | X4 | D | Y10 | H | H | H | D | D |
| LM61 | Y10 | D | X4 | D | Y10 | D | H | H | D | D |
| LM62 | Y10 | D | X4 | D | Y10 | D | D | H | D | D |
| LM63 | Y10 | D | X4 | D | Y10 | D | D | D | D | D |
| LM64 | Y10 | D | Y12 | D | Y10 | D | D | D | D | D |
| LM65 | Y10 | D | Y12 | D | Y10 | H | X1 | H | D | D |
| LM66 | Y10 | D | Y12 | D | Y10 | D | Y3 | D | D | D |
| LM67 | Y10 | D | Y12 | D | Y10 | H | X4 | H | D | D |
| LM68 | Y10 | D | Y12 | D | Y10 | D | Y12 | D | D | D |
| LM69 | X1 | H | X5 | H | X1 | H | H | H | H | D |
| LM70 | X1 | H | X5 | H | X1 | H | H | H | D | H |
| LM71 | X1 | H | X5 | H | X1 | H | H | H | D | D |
| LM72 | Y1 | H | X5 | H | Y1 | H | H | H | D | D |
| LM73 | Y2 | H | X5 | H | Y2 | H | H | H | D | D |
| LM74 | Y3 | H | X5 | H | Y3 | H | H | H | D | D |
| LM75 | Y3 | D | X5 | D | Y3 | H | H | H | D | D |
| LM76 | Y3 | D | X5 | D | Y3 | D | H | H | D | D |
| LM77 | Y3 | D | X5 | D | Y3 | D | D | H | D | D |
| LM78 | Y3 | D | X5 | D | Y3 | D | D | D | D | D |
| LM79 | Y3 | D | Y13 | D | Y3 | D | D | D | D | D |
| LM80 | Y3 | D | Y13 | D | Y3 | H | X1 | H | D | D |
| LM81 | Y3 | D | Y13 | D | Y3 | D | Y3 | D | D | D |
| LM82 | Y3 | D | Y13 | D | Y3 | H | X4 | H | D | D |
| LM83 | Y3 | D | Y13 | D | Y3 | D | Y12 | D | D | D |
| LM84 | X2 | H | X5 | H | X2 | H | H | H | H | D |
| LM85 | X2 | H | X5 | H | X2 | H | H | H | D | H |
| LM86 | X2 | H | X5 | H | X2 | H | H | H | D | D |
| LM87 | Y4 | H | X5 | H | Y4 | H | H | H | D | D |
| LM88 | Y5 | H | X5 | H | Y5 | H | H | H | D | D |
| LM89 | Y6 | H | X5 | H | Y6 | H | H | H | D | D |
| LM90 | Y7 | H | X5 | H | Y7 | H | H | H | D | D |
| LM91 | Y8 | H | X5 | H | Y8 | H | H | H | D | D |
| LM92 | Y9 | H | X5 | H | Y9 | H | H | H | D | D |
| LM93 | Y10 | H | X5 | H | Y10 | H | H | H | D | D |
| LM94 | Y10 | D | X5 | D | Y10 | H | H | H | D | D |
| LM95 | Y10 | D | X5 | D | Y10 | D | H | H | D | D |
| LM96 | Y10 | D | X5 | D | Y10 | D | D | H | D | D |
| LM97 | Y10 | D | X5 | D | Y10 | D | D | D | D | D |
| LM98 | Y10 | D | Y13 | D | Y10 | D | D | D | D | D |
| LM99 | Y10 | D | Y13 | D | Y10 | H | X1 | H | D | D |
| LM100 | Y10 | D | Y13 | D | Y10 | D | Y3 | D | D | D |
| LM101 | Y10 | D | Y13 | D | Y10 | H | X4 | H | D | D |
| LM102 | Y10 | D | Y13 | D | Y10 | D | Y12 | D | D | D |
| LM103 | X1 | H | X6 | H | X1 | H | H | H | H | D |
| LM104 | X1 | H | X6 | H | X1 | H | H | H | D | H |
| LM105 | X1 | H | X6 | H | X1 | H | H | H | D | D |
| LM106 | Y1 | H | X6 | H | Y1 | H | H | H | D | D |
| LM107 | Y2 | H | X6 | H | Y2 | H | H | H | D | D |
| LM108 | Y3 | H | X6 | H | Y3 | H | H | H | D | D |
| LM109 | Y3 | D | X6 | D | Y3 | H | H | H | D | D |
| LM110 | Y3 | D | X6 | D | Y3 | D | H | H | D | D |
| LM111 | Y3 | D | X6 | D | Y3 | D | D | H | D | D |
| LM112 | Y3 | D | X6 | D | Y3 | D | D | D | D | D |
| LM113 | Y3 | D | Y14 | D | Y3 | D | D | D | D | D |
| LM114 | Y3 | D | Y14 | D | Y3 | H | X1 | H | D | D |
| LM115 | Y3 | D | Y14 | D | Y3 | D | Y3 | D | D | D |
| LM116 | Y3 | D | Y14 | D | Y3 | H | X4 | H | D | D |
| LM117 | Y3 | D | Y14 | D | Y3 | D | Y12 | D | D | D |
| LM118 | X2 | H | X6 | H | X2 | H | H | H | H | D |
| LM119 | X2 | H | X6 | H | X2 | H | H | H | D | H |
| LM120 | X2 | H | X6 | H | X2 | H | H | H | D | D |
| LM121 | Y4 | H | X6 | H | Y4 | H | H | H | D | D |
| LM122 | Y5 | H | X6 | H | Y5 | H | H | H | D | D |
| LM123 | Y6 | H | X6 | H | Y6 | H | H | H | D | D |
| LM124 | Y7 | H | X6 | H | Y7 | H | H | H | D | D |
| LM125 | Y8 | H | X6 | H | Y8 | H | H | H | D | D |
| LM126 | Y9 | H | X6 | H | Y9 | H | H | H | D | D |
| LM127 | Y10 | H | X6 | H | Y10 | H | H | H | D | D |
| LM128 | Y10 | D | X6 | D | Y10 | H | H | H | D | D |
| LM129 | Y10 | D | X6 | D | Y10 | D | H | H | D | D |
| LM130 | Y10 | D | X6 | D | Y10 | D | D | H | D | D |
| LM131 | Y10 | D | X6 | D | Y10 | D | D | D | D | D |
| LM132 | Y10 | D | Y14 | D | Y10 | D | D | D | D | D |
| LM133 | Y10 | D | Y14 | D | Y10 | H | X1 | H | D | D |
| LM134 | Y10 | D | Y14 | D | Y10 | D | Y3 | D | D | D |
| LM135 | Y10 | D | Y14 | D | Y10 | H | X4 | H | D | D |
| LM136 | Y10 | D | Y14 | D | Y10 | D | Y12 | D | D | D |
| LM137 | X1 | H | X7 | H | X1 | H | H | H | H | D |
| LM138 | X1 | H | X7 | H | X1 | H | H | H | D | H |
| LM139 | X1 | H | X7 | H | X1 | H | H | H | D | D |
| LM140 | Y1 | H | X7 | H | Y1 | H | H | H | D | D |
| LM141 | Y2 | H | X7 | H | Y2 | H | H | H | D | D |
| LM142 | Y3 | H | X7 | H | Y3 | H | H | H | D | D |
| LM143 | Y3 | D | X7 | D | Y3 | H | H | H | D | D |
| LM144 | Y3 | D | X7 | D | Y3 | D | H | H | D | D |
| LM145 | Y3 | D | X7 | D | Y3 | D | D | H | D | D |
| LM146 | Y3 | D | X7 | D | Y3 | D | D | D | D | D |
| LM147 | Y3 | D | X8 | D | Y3 | D | D | D | D | D |
| LM148 | Y3 | D | Y16 | D | Y3 | D | D | D | D | D |
| LM149 | Y3 | D | Y17 | D | Y3 | D | D | D | D | D |
| LM150 | Y3 | D | Y18 | D | Y3 | D | D | D | D | D |
| LM151 | Y3 | D | Y15 | D | Y3 | D | D | D | D | D |
| LM152 | Y3 | D | Y15 | D | Y3 | H | X1 | H | D | D |
| LM153 | Y3 | D | Y15 | D | Y3 | D | Y3 | D | D | D |
| LM154 | Y3 | D | Y16 | D | Y3 | D | Y3 | D | D | D |
| LM155 | Y3 | D | Y17 | D | Y3 | D | Y3 | D | D | D |
| LM156 | Y3 | D | Y18 | D | Y3 | D | Y3 | D | D | D |
| LM157 | Y3 | D | Y15 | D | Y3 | H | X4 | H | D | D |
| LM158 | Y3 | D | Y15 | D | Y3 | D | Y12 | D | D | D |
| LM159 | Y3 | D | Y16 | D | Y3 | D | Y12 | D | D | D |
| LM160 | Y3 | D | Y17 | D | Y3 | D | Y12 | D | D | D |
| LM161 | Y3 | D | Y18 | D | Y3 | D | Y12 | D | D | D |
| LM162 | X2 | H | X7 | H | X2 | H | H | H | H | D |
| LM163 | X2 | H | X7 | H | X2 | H | H | H | D | H |
| LM164 | X2 | H | X7 | H | X2 | H | H | H | D | D |
| LM165 | Y4 | H | X7 | H | Y4 | H | H | H | D | D |
| LM166 | Y5 | H | X7 | H | Y5 | H | H | H | D | D |
| LM167 | Y6 | H | X7 | H | Y6 | H | H | H | D | D |
| LM168 | Y7 | H | X7 | H | Y7 | H | H | H | D | D |
| LM169 | Y8 | H | X7 | H | Y8 | H | H | H | D | D |
| LM170 | Y9 | H | X7 | H | Y9 | H | H | H | D | D |
| LM171 | Y10 | H | X7 | H | Y10 | H | H | H | D | D |
| LM172 | Y10 | D | X7 | D | Y10 | H | H | H | D | D |
| LM173 | Y10 | D | X7 | D | Y10 | D | H | H | D | D |
| LM174 | Y10 | D | X7 | D | Y10 | D | D | H | D | D |
| LM175 | Y10 | D | X7 | D | Y10 | D | D | D | D | D |
| LM176 | Y10 | D | X8 | D | Y10 | D | D | D | D | D |
| LM177 | Y10 | D | Y16 | D | Y10 | D | D | D | D | D |
| LM178 | Y10 | D | Y17 | D | Y10 | D | D | D | D | D |
| LM179 | Y10 | D | Y18 | D | Y10 | D | D | D | D | D |
| LM180 | Y10 | D | Y15 | D | Y10 | D | D | D | D | D |
| LM181 | Y10 | D | Y15 | D | Y10 | H | X1 | H | D | D |
| LM182 | Y10 | D | Y15 | D | Y10 | D | Y3 | D | D | D |
| LM183 | Y10 | D | Y16 | D | Y10 | D | Y3 | D | D | D |
| LM184 | Y10 | D | Y17 | D | Y10 | D | Y3 | D | D | D |
| LM185 | Y10 | D | Y18 | D | Y10 | D | Y3 | D | D | D |
| LM186 | Y10 | D | Y15 | D | Y10 | H | X4 | H | D | D |
| LM187 | Y10 | D | Y15 | D | Y10 | D | Y12 | D | D | D |
| LM188 | Y10 | D | Y16 | D | Y10 | D | Y12 | D | D | D |
| LM189 | Y10 | D | Y17 | D | Y10 | D | Y12 | D | D | D |
| LM190 | Y10 | D | Y18 | D | Y10 | D | Y12 | D | D | D |
| LM191 | X1 | X7 | H | H | X1 | H | H | H | H | D |
| LM192 | X | X7 | H | H | X1 | H | H | H | D | H |
| LM193 | X1 | X7 | H | H | X1 | H | H | H | D | D |
| LM194 | Y1 | X7 | H | H | Y1 | H | H | H | D | D |
| LM195 | Y2 | X7 | H | H | Y2 | H | H | H | D | D |
| LM196 | Y3 | X7 | H | H | Y3 | H | H | H | D | D |
| LM197 | Y3 | X7 | D | D | Y3 | H | H | H | D | D |
| LM198 | Y3 | X7 | D | D | Y3 | D | H | H | D | D |
| LM199 | Y3 | X7 | D | D | Y3 | D | D | H | D | D |
| LM200 | Y3 | X7 | D | D | Y3 | D | D | D | D | D |
| LM201 | Y3 | Y15 | D | D | Y3 | D | D | D | D | D |
| LM202 | Y3 | Y16 | D | D | Y3 | D | D | D | D | D |
| LM203 | Y3 | Y17 | D | D | Y3 | D | D | D | D | D |
| LM204 | Y3 | Y18 | D | D | Y3 | D | D | D | D | D |
| LM205 | Y3 | Y15 | D | D | Y3 | H | X1 | H | D | D |
| LM206 | Y3 | Y15 | D | D | Y3 | D | Y3 | D | D | D |
| LM207 | Y3 | Y16 | D | D | Y3 | D | Y3 | D | D | D |
| LM208 | Y3 | Y17 | D | D | Y3 | D | Y3 | D | D | D |
| LM209 | Y3 | Y18 | D | D | Y3 | D | Y3 | D | D | D |
| LM210 | Y3 | Y15 | D | D | Y3 | H | X4 | H | D | D |
| LM211 | Y3 | Y15 | D | D | Y3 | D | Y12 | D | D | D |
| LM212 | Y3 | Y16 | D | D | Y3 | D | Y12 | D | D | D |
| LM213 | Y3 | Y17 | D | D | Y3 | D | Y12 | D | D | D |
| LM214 | Y3 | Y18 | D | D | Y3 | D | Y12 | D | D | D |
| LM215 | X2 | X7 | H | H | X2 | H | H | H | H | D |
| LM216 | X2 | X7 | H | H | X2 | H | H | H | D | H |
| LM217 | X2 | X7 | H | H | X2 | H | H | H | D | D |
| LM218 | Y4 | X7 | H | H | Y4 | H | H | H | D | D |
| LM219 | Y5 | X7 | H | H | Y5 | H | H | H | D | D |
| LM220 | Y6 | X7 | H | H | Y6 | H | H | H | D | D |
| LM221 | Y7 | X7 | H | H | Y7 | H | H | H | D | D |
| LM222 | Y8 | X7 | H | H | Y8 | H | H | H | D | D |
| LM223 | Y9 | X7 | H | H | Y9 | H | H | H | D | D |
| LM224 | Y10 | X7 | H | H | Y10 | H | H | H | D | D |
| LM225 | Y10 | X7 | D | D | Y10 | H | H | H | D | D |
| LM226 | Y10 | X7 | D | D | Y10 | D | H | H | D | D |
| LM227 | Y10 | X7 | D | D | Y10 | D | D | H | D | D |
| LM228 | Y10 | X7 | D | D | Y10 | D | D | D | D | D |
| LM229 | Y10 | X8 | D | D | Y10 | D | D | D | D | D |
| LM230 | Y10 | Y16 | D | D | Y10 | D | D | D | D | D |
| LM231 | Y10 | Y17 | D | D | Y10 | D | D | D | D | D |
| LM232 | Y10 | Y18 | D | D | Y10 | D | D | D | D | D |
| LM233 | Y10 | Y15 | D | D | Y10 | D | D | D | D | D |
| LM234 | Y10 | Y15 | D | D | Y10 | H | X1 | H | D | D |
| LM235 | Y10 | Y15 | D | D | Y10 | D | Y3 | D | D | D |
| LM236 | Y10 | Y16 | D | D | Y10 | D | Y3 | D | D | D |
| LM237 | Y10 | Y17 | D | D | Y10 | D | Y3 | D | D | D |
| LM238 | Y10 | Y18 | D | D | Y10 | D | Y3 | D | D | D |
| LM239 | Y10 | Y15 | D | D | Y10 | H | X4 | H | D | D |
| LM240 | Y10 | Y15 | D | D | Y10 | D | Y12 | D | D | D |
| LM241 | Y10 | Y16 | D | D | Y10 | D | Y12 | D | D | D |
| LM242 | Y10 | Y17 | D | D | Y10 | D | Y12 | D | D | D |
| LM243 | Y10 | Y18 | D | D | Y10 | D | Y12 | D | D | D |

TABLE 5
Formula 1-2

| Ligand | | | | | | | | | | | | | |
|---|---|---|---|---|---|---|---|---|---|---|---|---|---|
| name | R11 | X11 | R101 | R102 | R103 | R104 | R14 | R15 | R16 | R17 | R18 | R19 | R20 |
| LFM1 | Y10 | N-Ph | D | D | D | D | D | Y10 | D | D | D | D | D |
| LFM2 | Y10 | S | D | D | D | D | D | Y10 | D | D | D | D | D |
| LFM3 | Y10 | O | D | D | D | D | D | Y10 | D | D | D | D | D |
| LFM4 | Y3 | O | D | D | D | D | D | Y3 | D | D | D | D | D |
| LFM5 | Y10 | O | D | D | D | D | D | Y10 | D | D | D | D | D |
| LFM6 | Y10 | O | D | D | D | D | D | Y10 | D | Y3 | D | D | D |
| LFM7 | Y10 | C | D | D | D | D | D | Y10 | D | Y12 | D | D | D |

TABLE 6
Formula 1-3

| Ligand | | | | | | | | | | | | | |
|---|---|---|---|---|---|---|---|---|---|---|---|---|---|
| name | R11 | X11 | R101 | R102 | R103 | R104 | R14 | R15 | R16 | R17 | R18 | R19 | R20 |
| LFP1 | Y10 | N-Ph | D | D | D | D | D | Y10 | D | D | D | D | D |
| LFP2 | Y10 | S | D | D | D | D | D | Y10 | D | D | D | D | D |
| LFP3 | Y10 | O | D | D | D | D | D | Y10 | D | D | D | D | D |
| LFP4 | Y3 | O | D | D | D | D | D | Y3 | D | D | D | D | D |
| LFP5 | Y10 | O | D | D | D | D | D | Y10 | D | D | D | D | D |
| LFP6 | Y10 | O | D | D | D | D | D | Y10 | D | Y3 | D | D | D |
| LFP7 | Y10 | O | D | D | D | D | D | Y10 | D | Y12 | D | D | D |

X₁ to X₁₀ and Y₁ to Y₁₈ in Tables 4 to 6 may each be the same as described below, and Ph in the tables refers to a phenyl group:

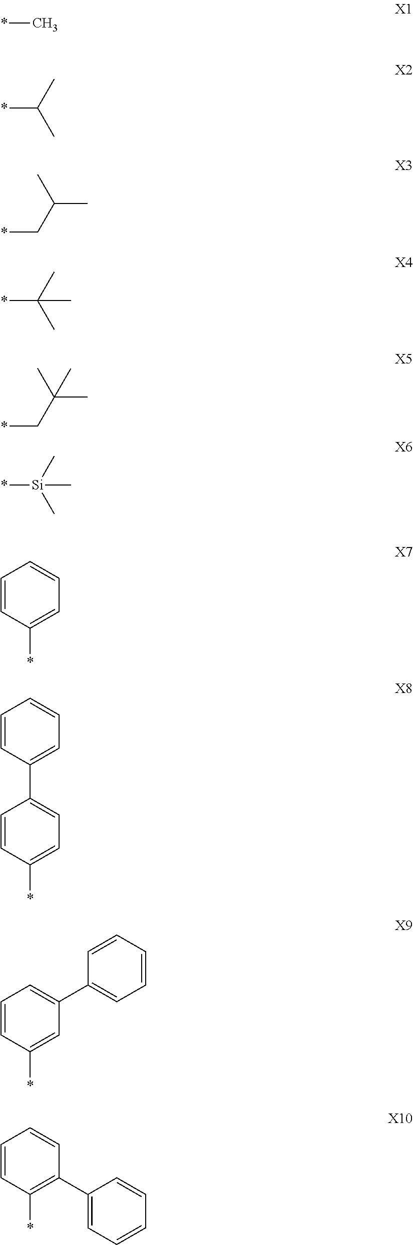

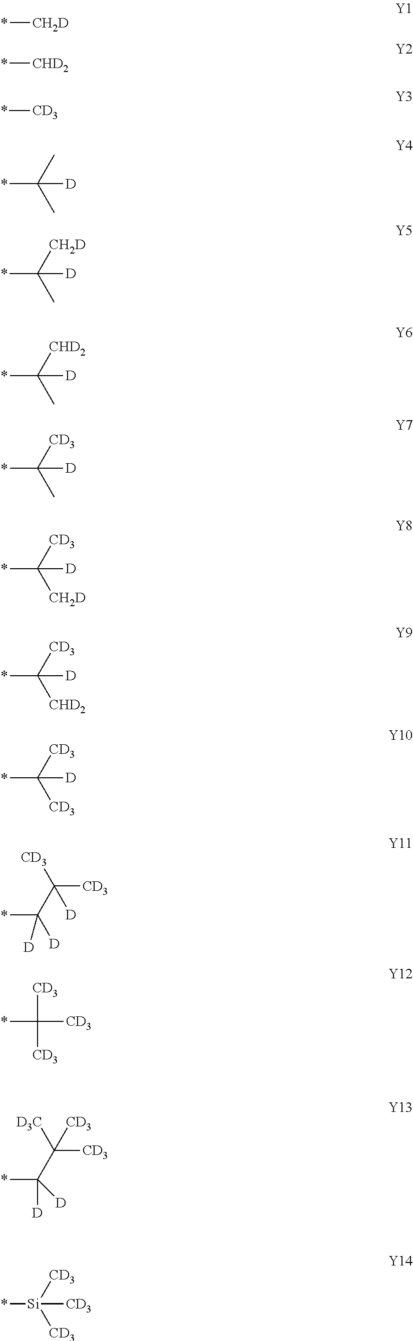

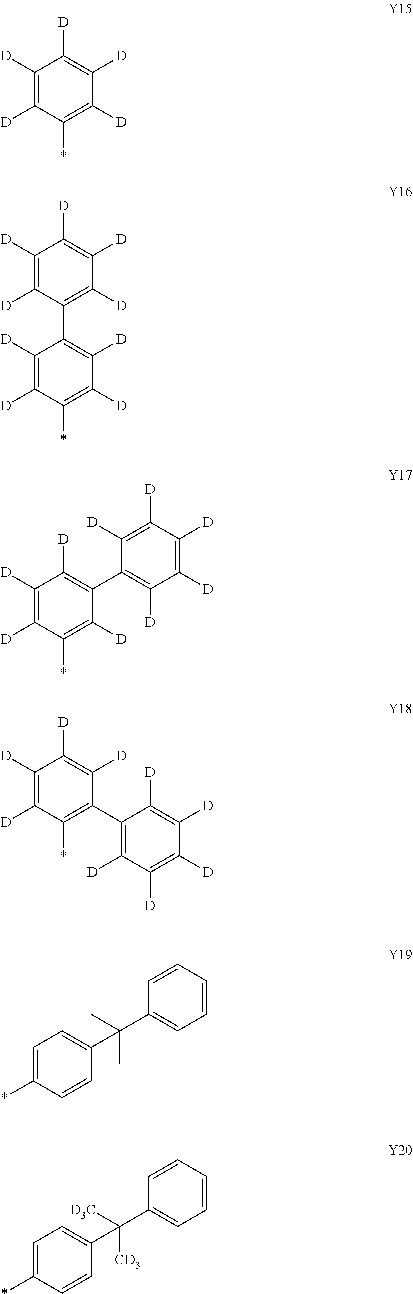 [SMILES: *c1c([2H])c([2H])c([2H])c([2H])c1[2H] ; *c1c([2H])c([2H])c(-c2c([2H])c([2H])c([2H])c([2H])c2[2H])c([2H])c1[2H] ; *c1c([2H])c([2H])c([2H])c(-c2c([2H])c([2H])c([2H])c([2H])c2[2H])c1[2H] ; *c1c([2H])c([2H])c([2H])c([2H])c1-c1c([2H])c([2H])c([2H])c([2H])c1[2H] ; *c1ccc(C(C)(C)c2ccccc2)cc1 ; *c1ccc(C(c2ccccc2)(C([2H])([2H])[2H])C([2H])([2H])[2H])cc1]

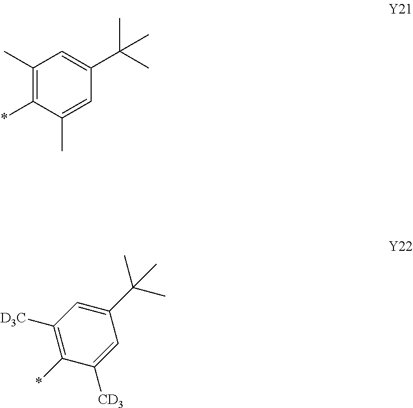 [SMILES: *c1c(C)cc(C(C)(C)C)cc1C ; *c1c(C([2H])([2H])[2H])cc(C(C)(C)C)cc1C([2H])([2H])[2H]]

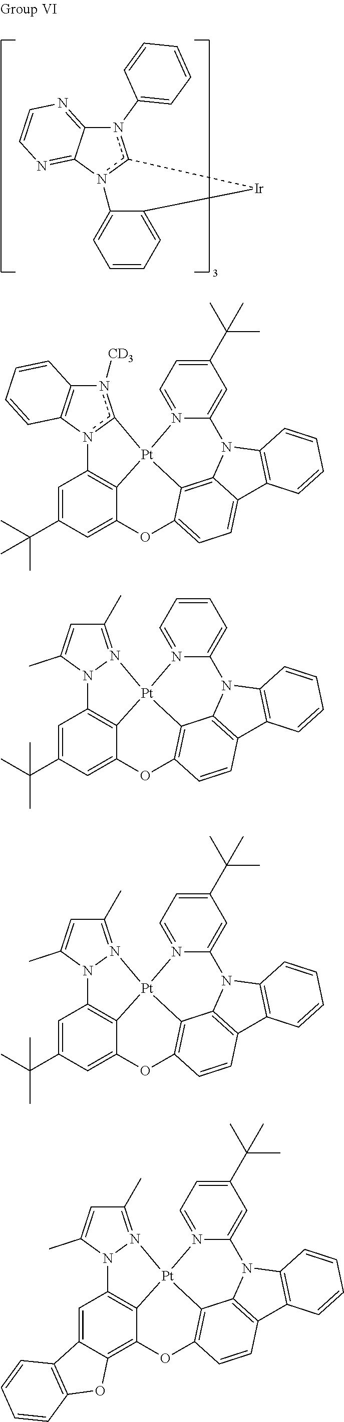

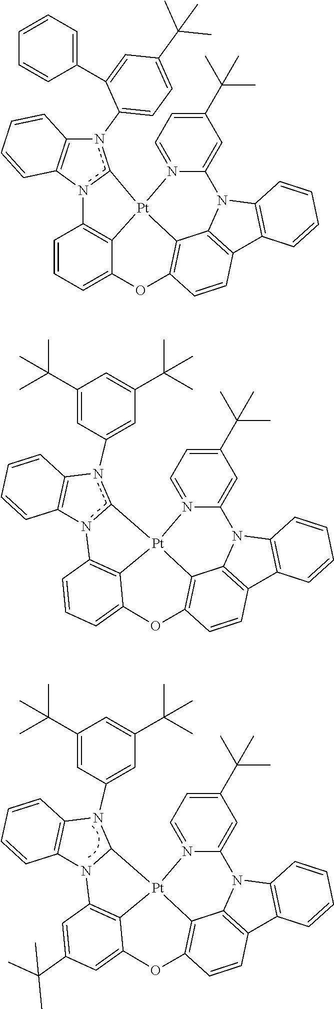

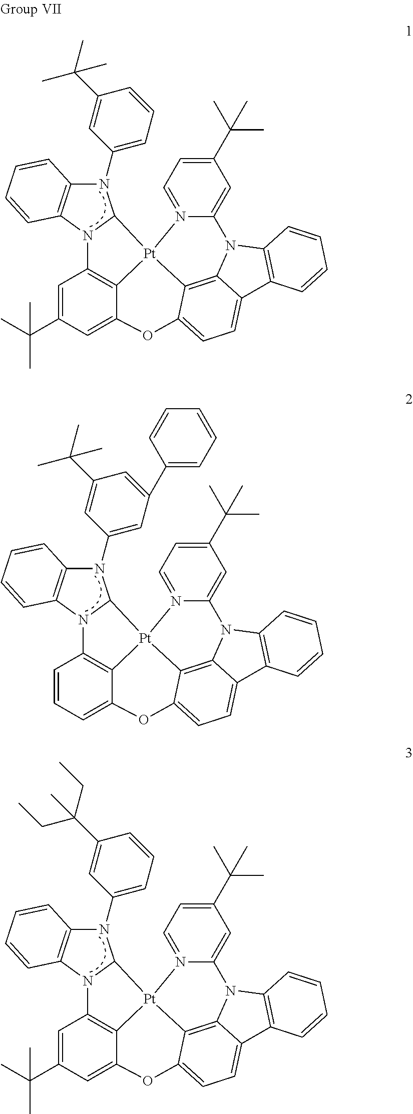

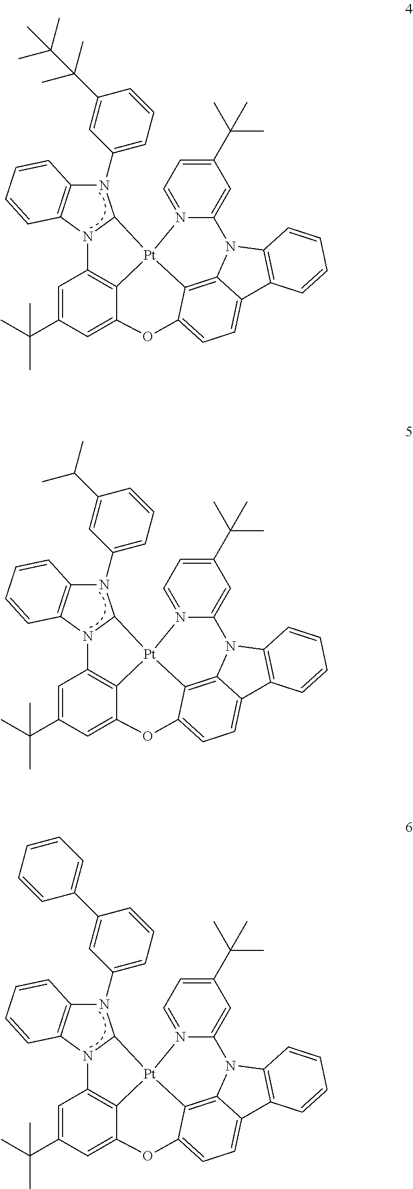

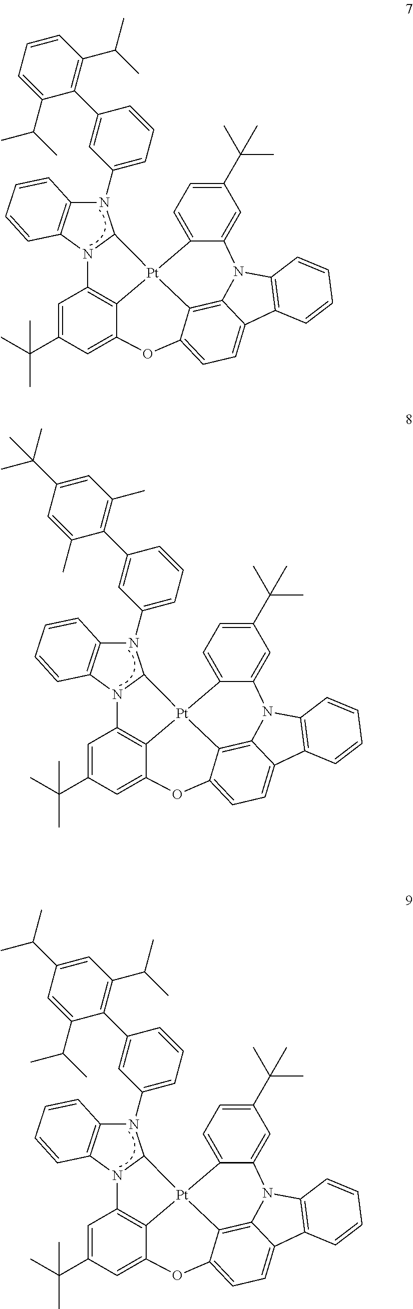

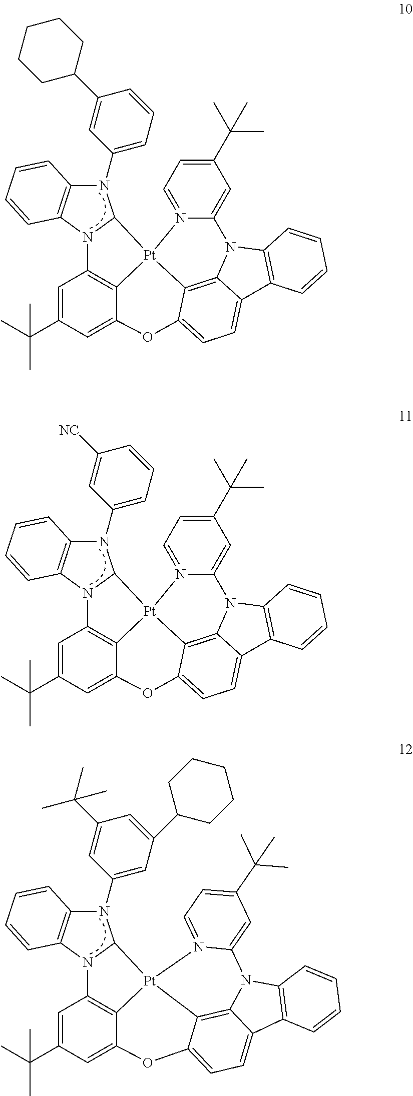

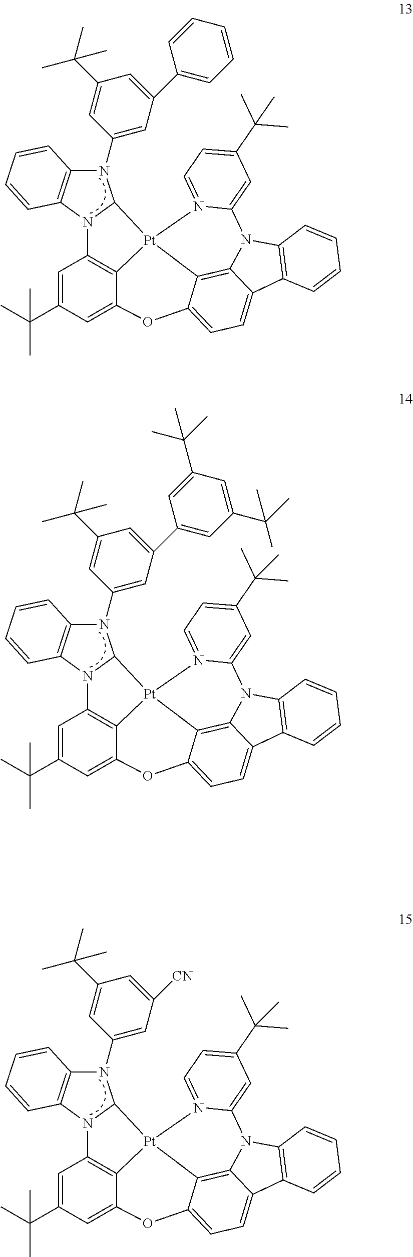

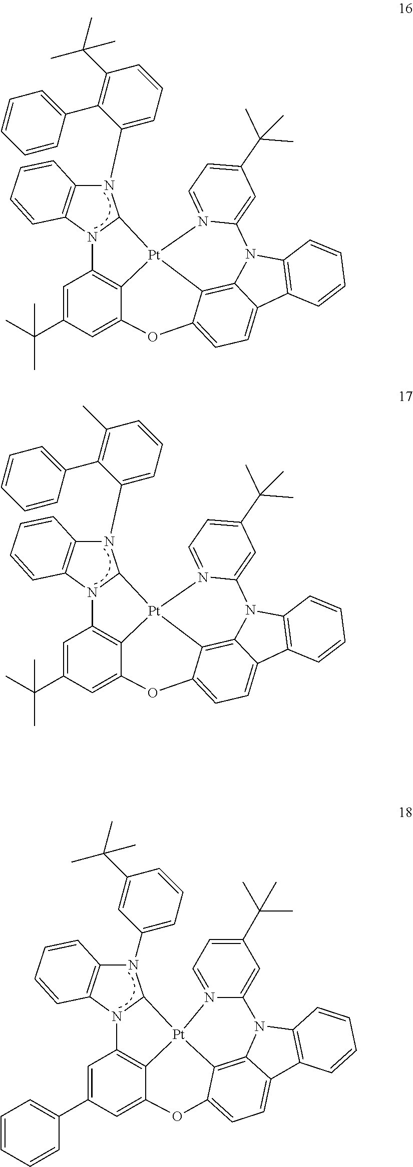

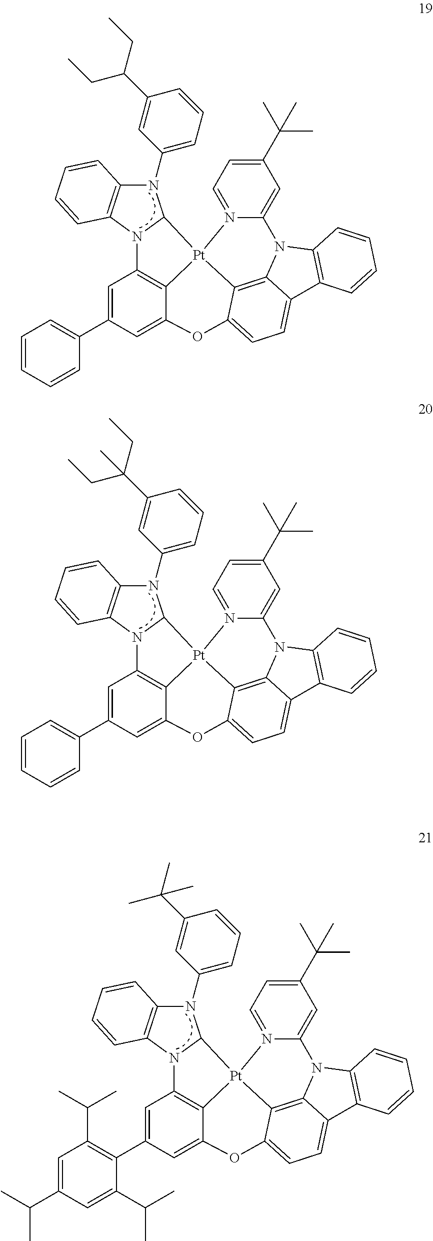

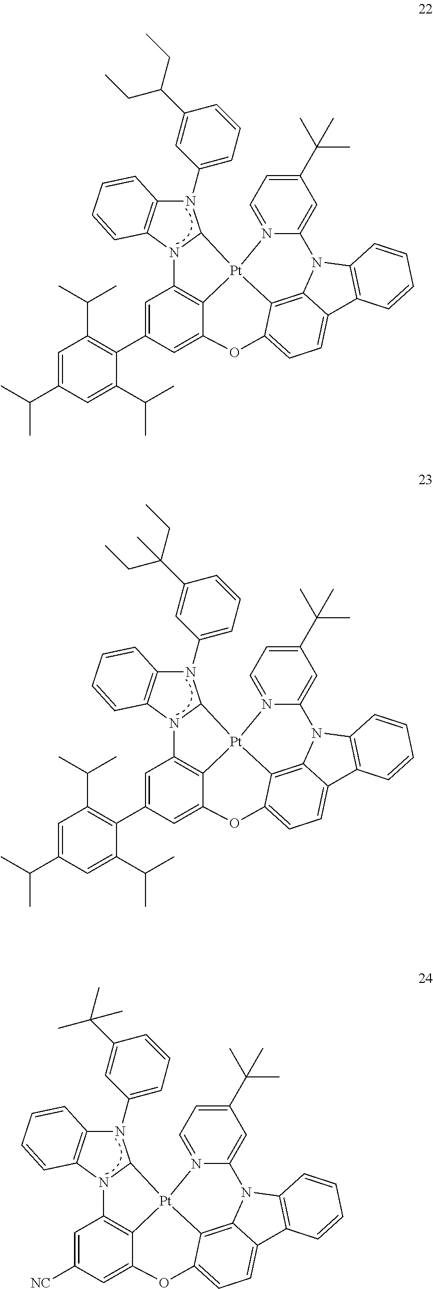

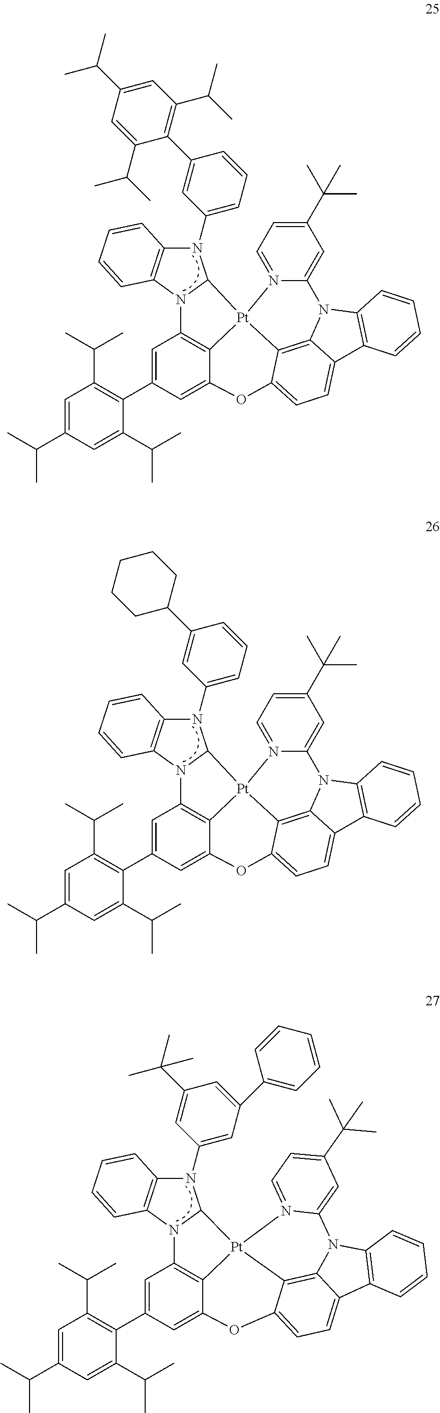

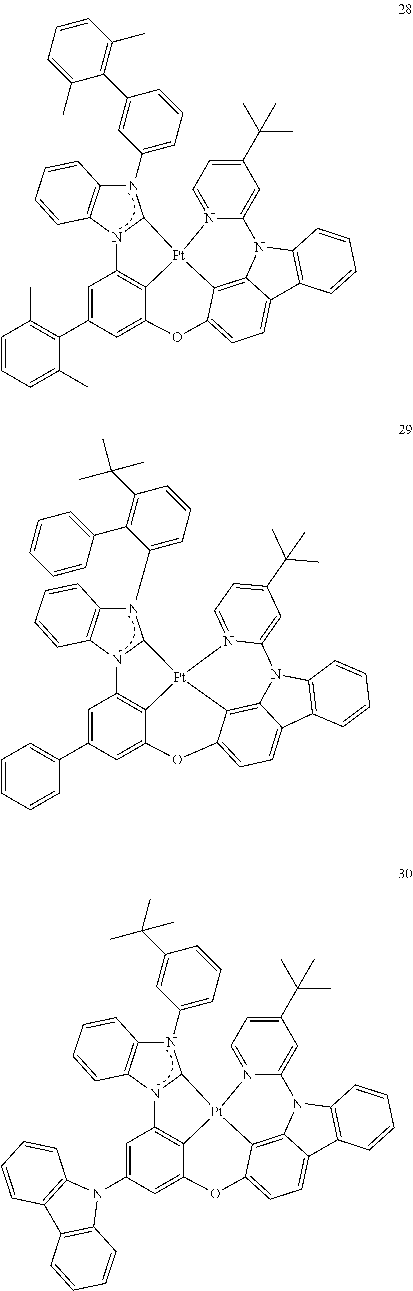

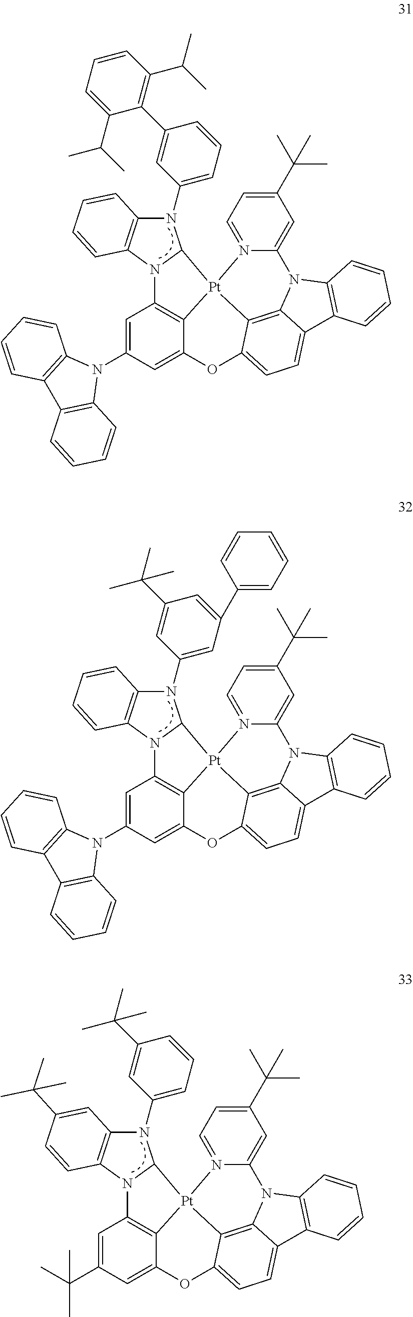

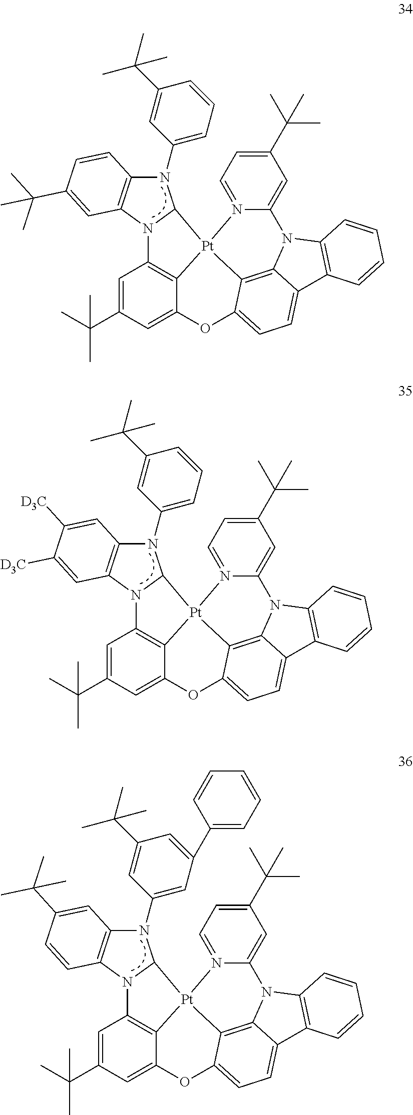

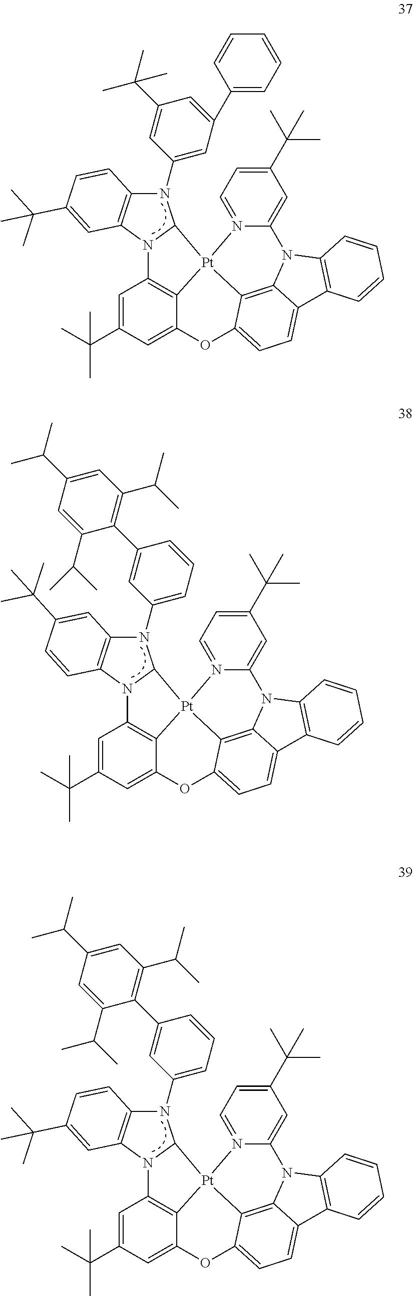

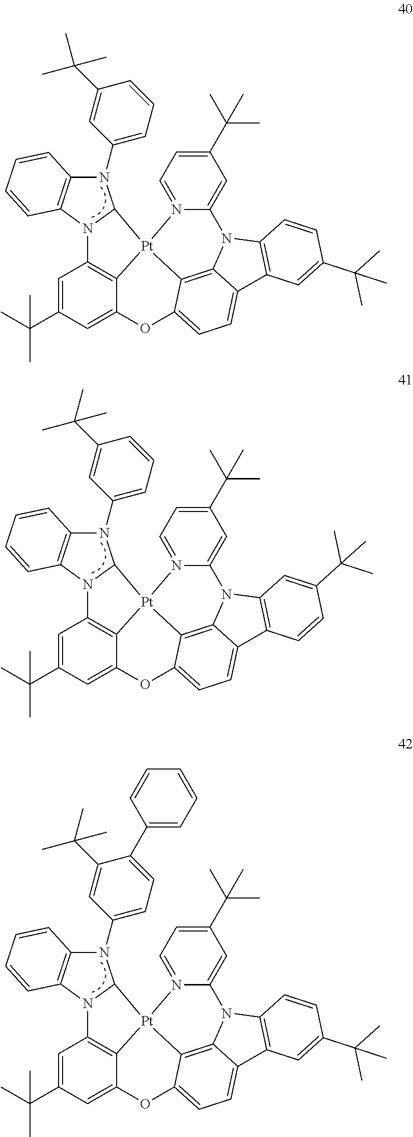

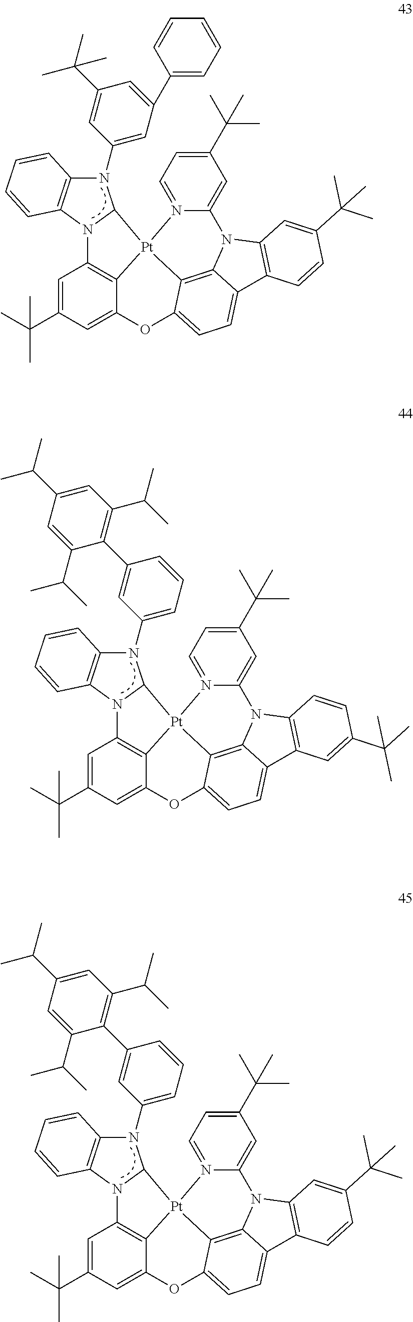

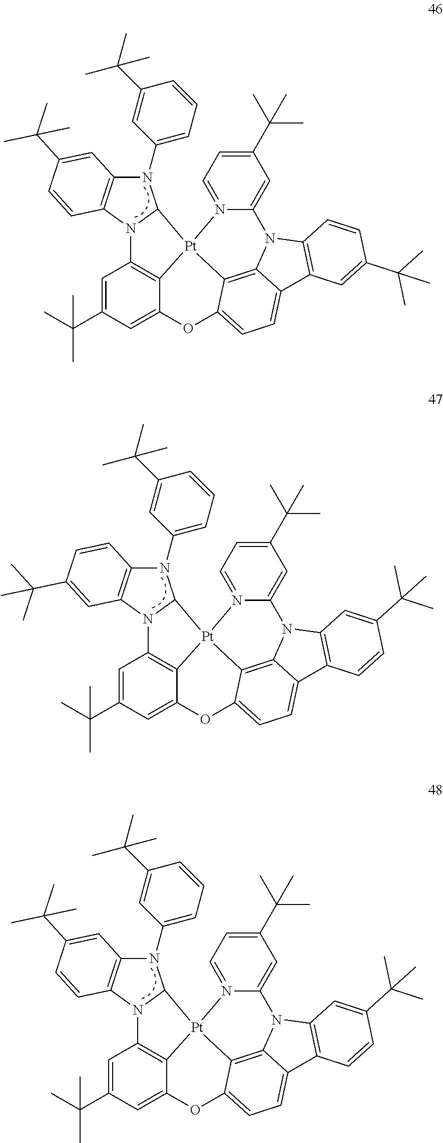

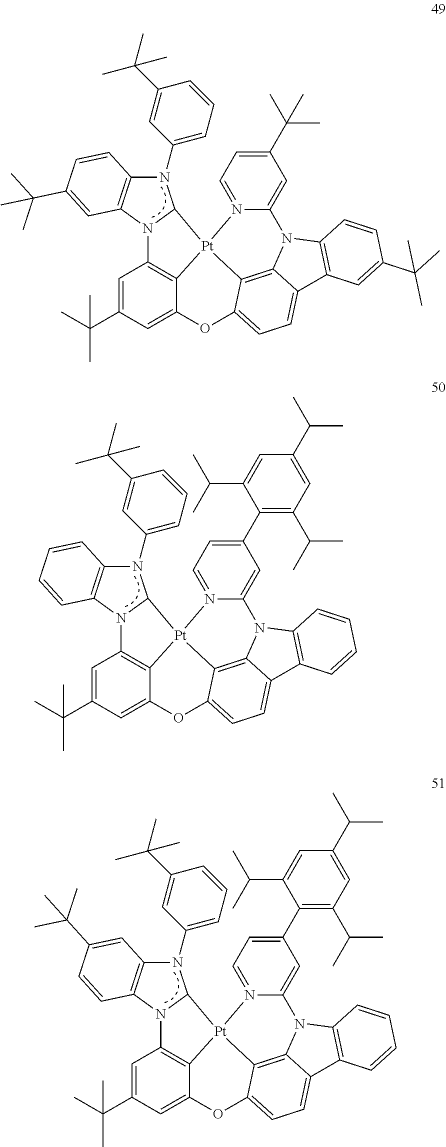

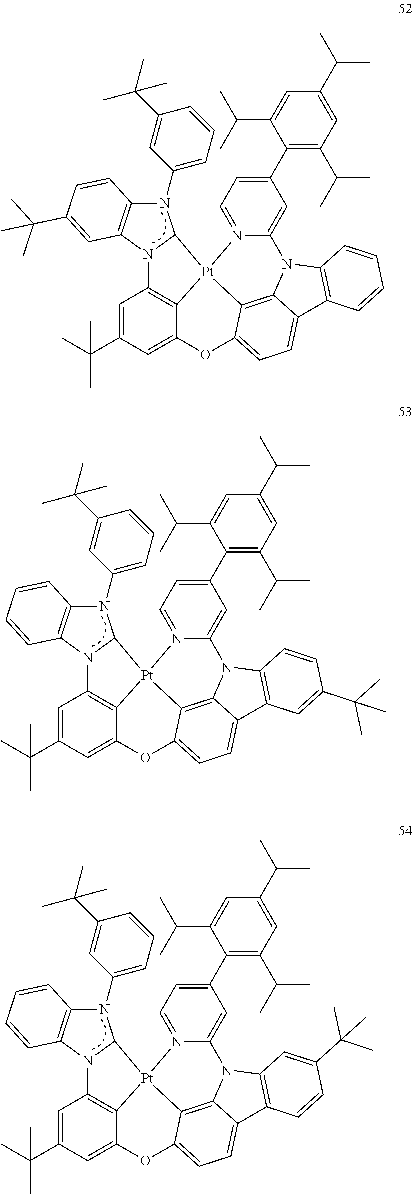

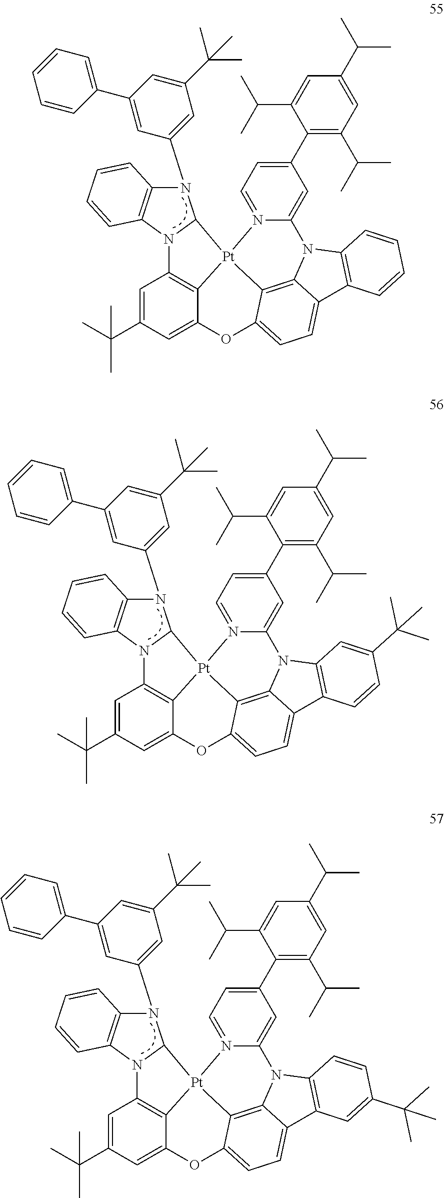

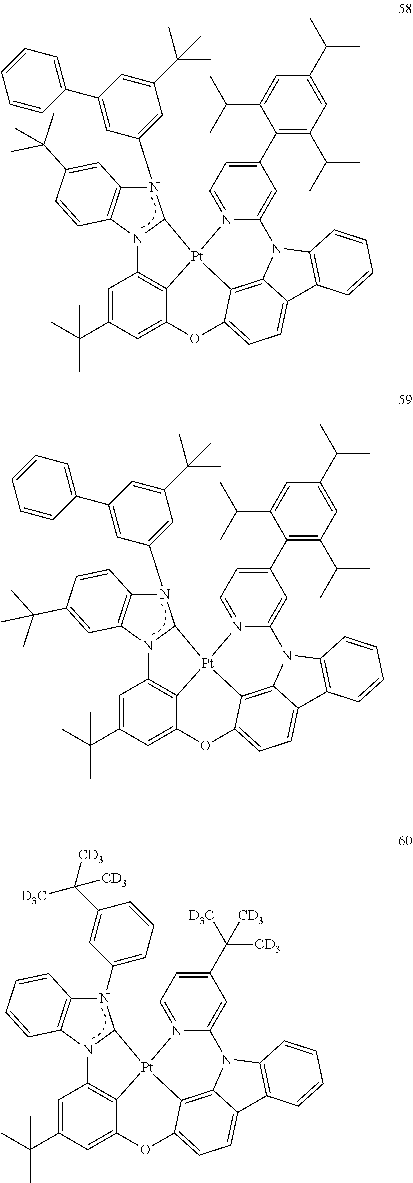

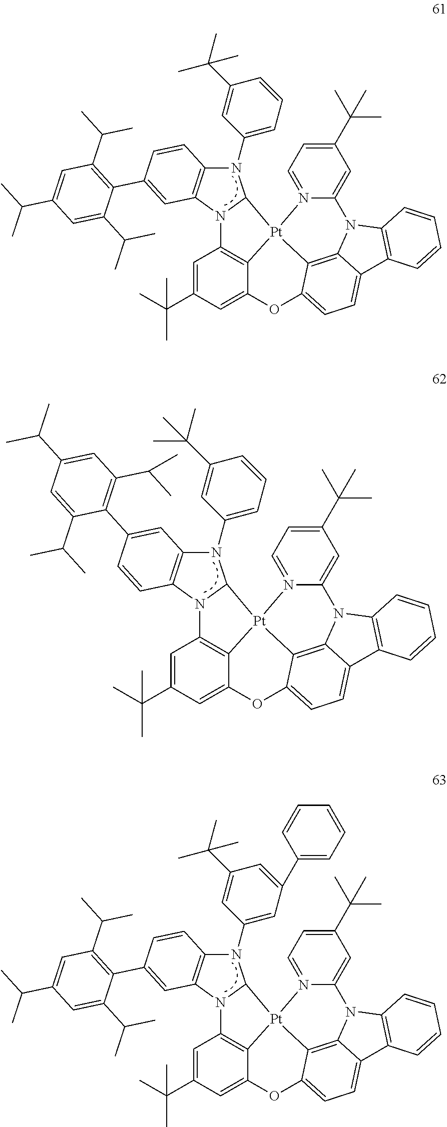

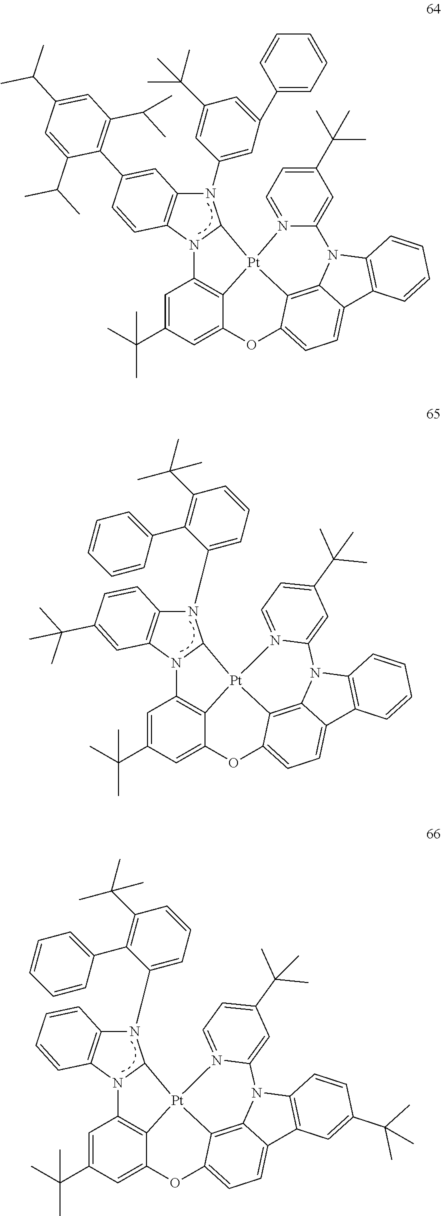

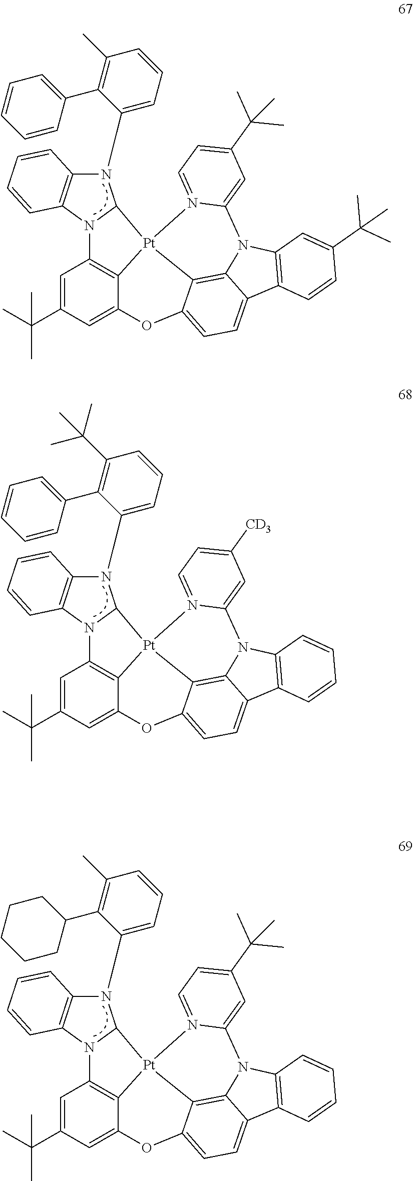

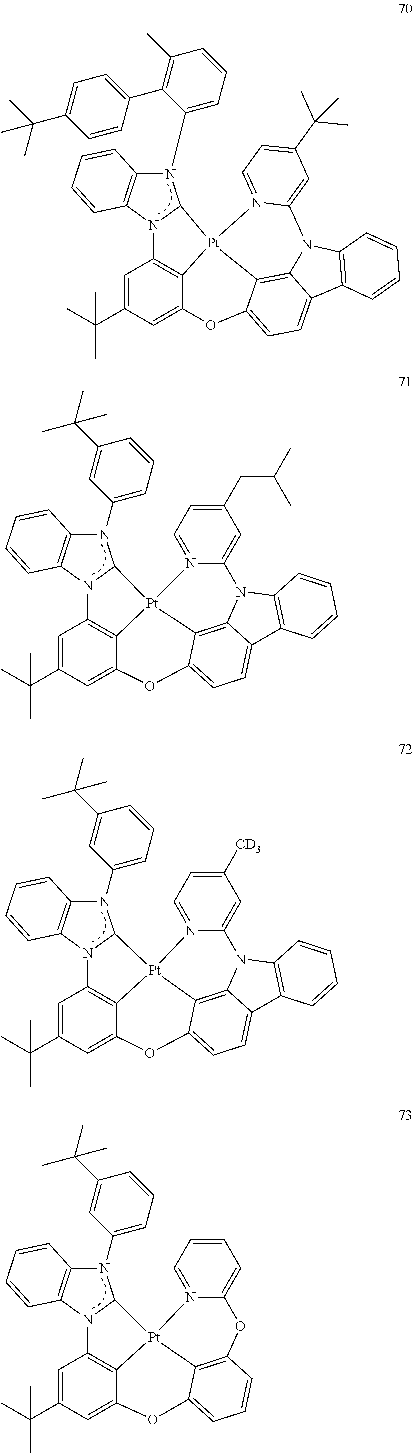

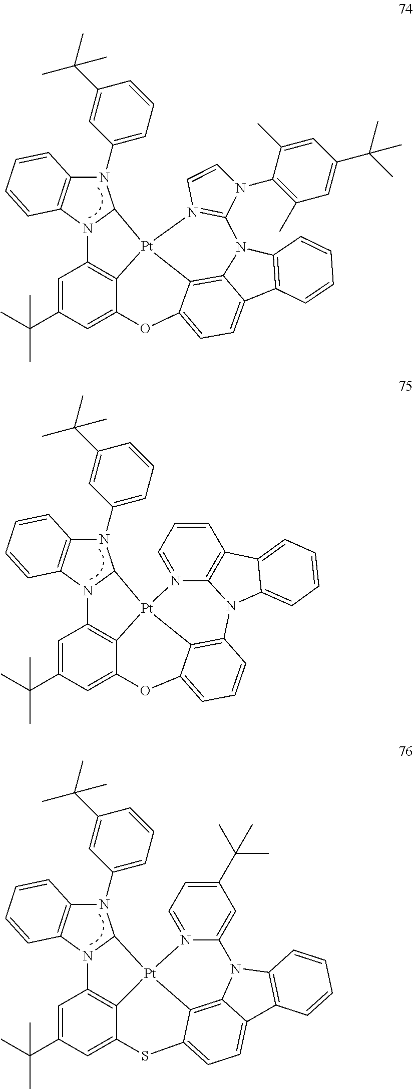

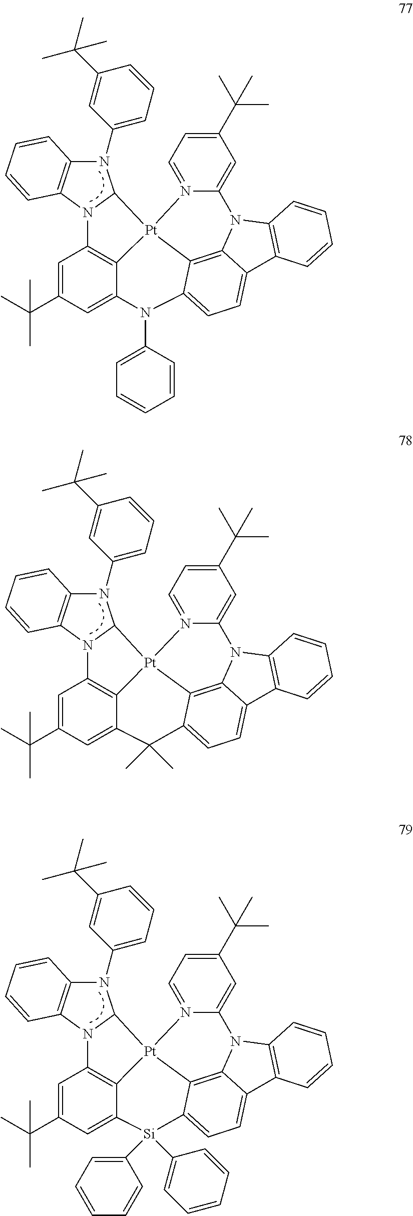

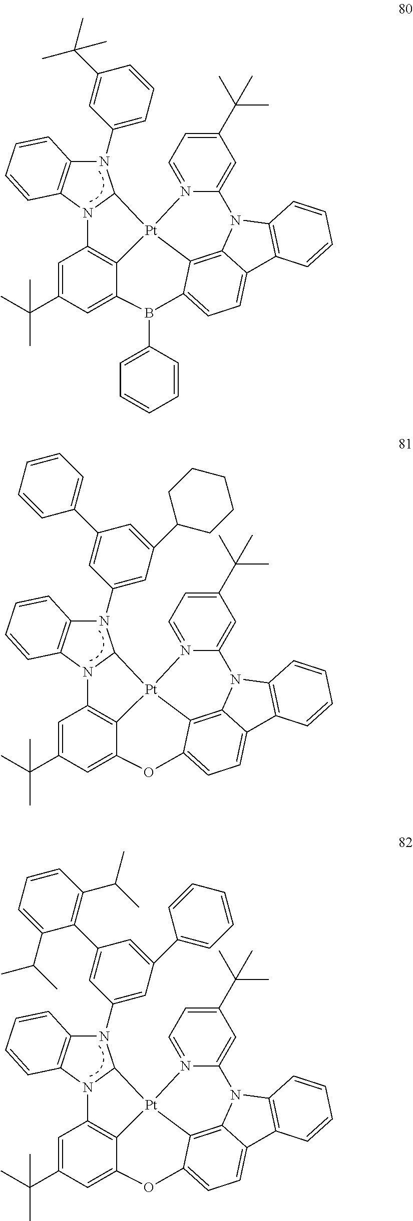

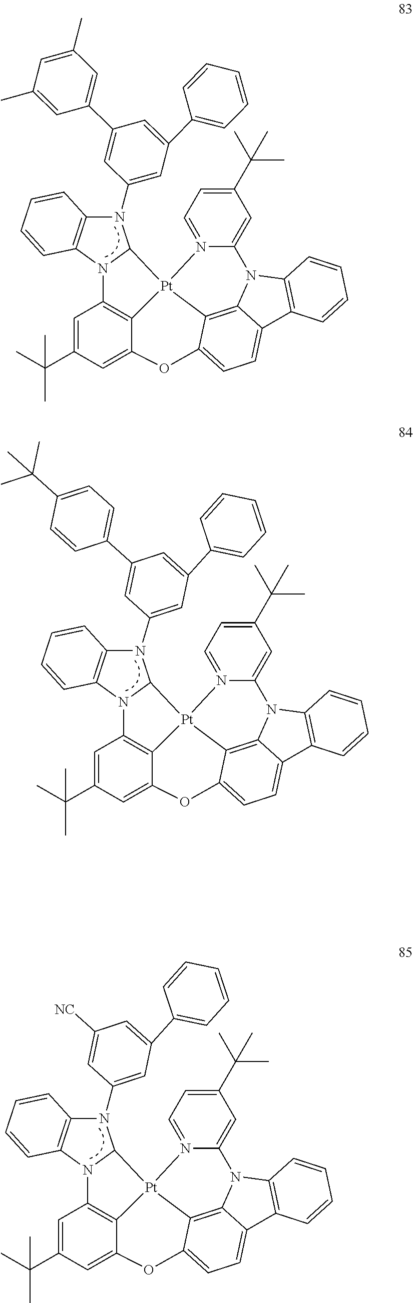

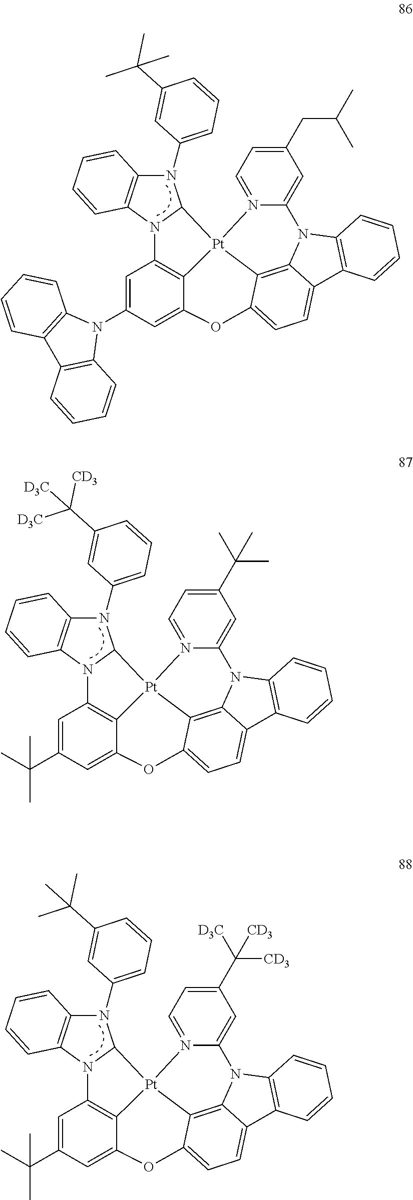

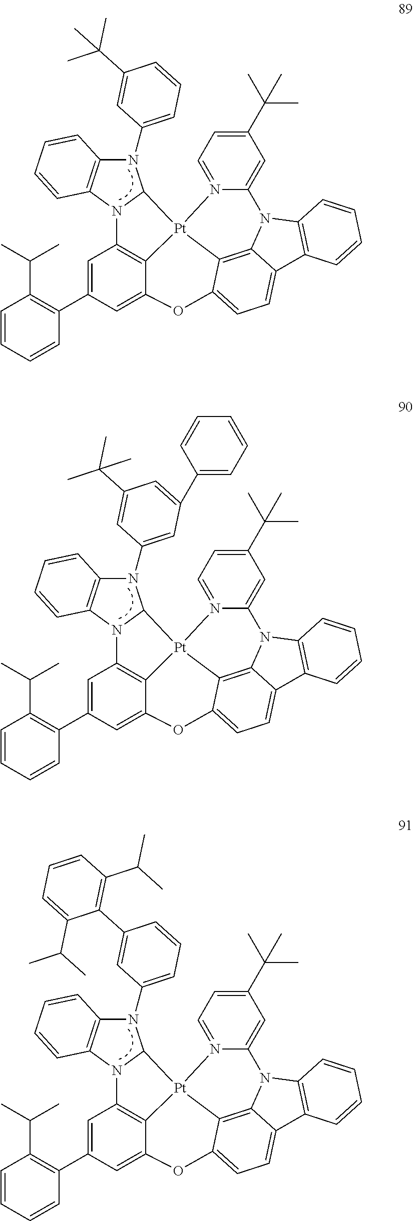

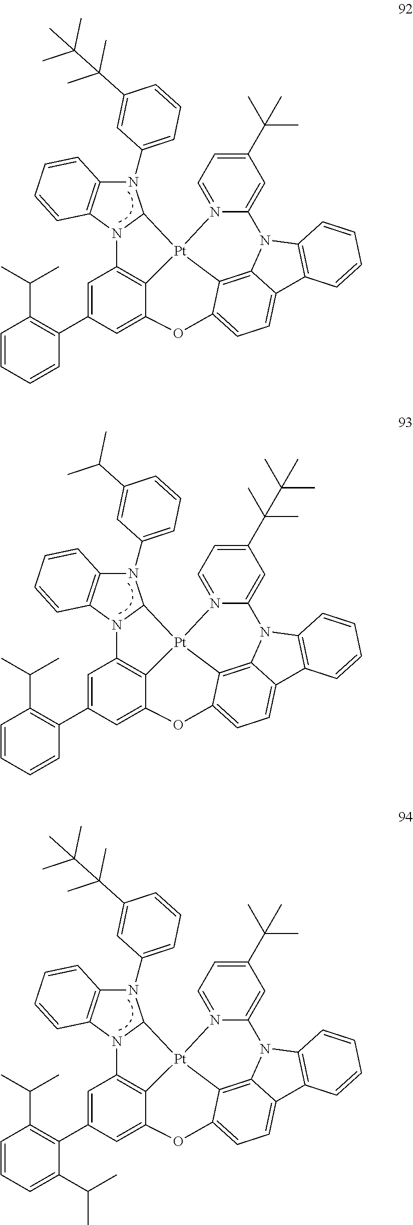

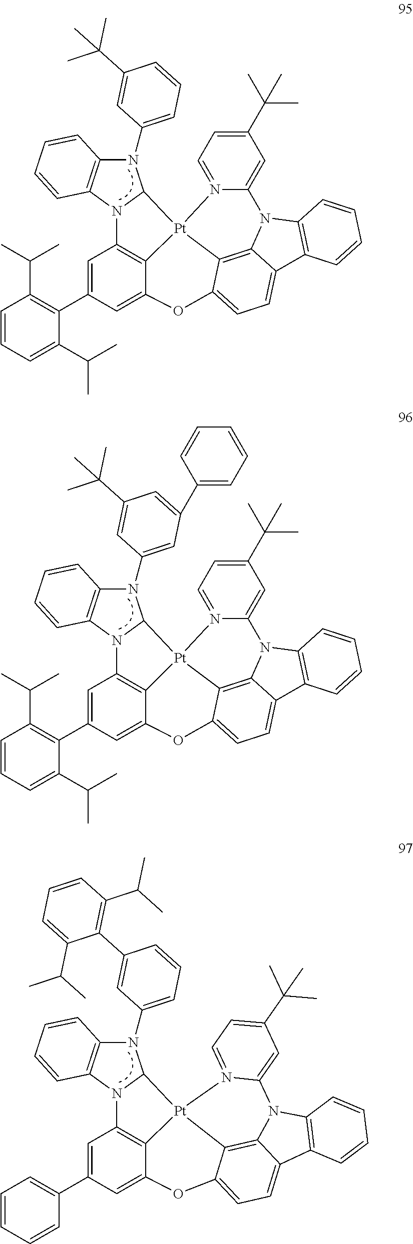

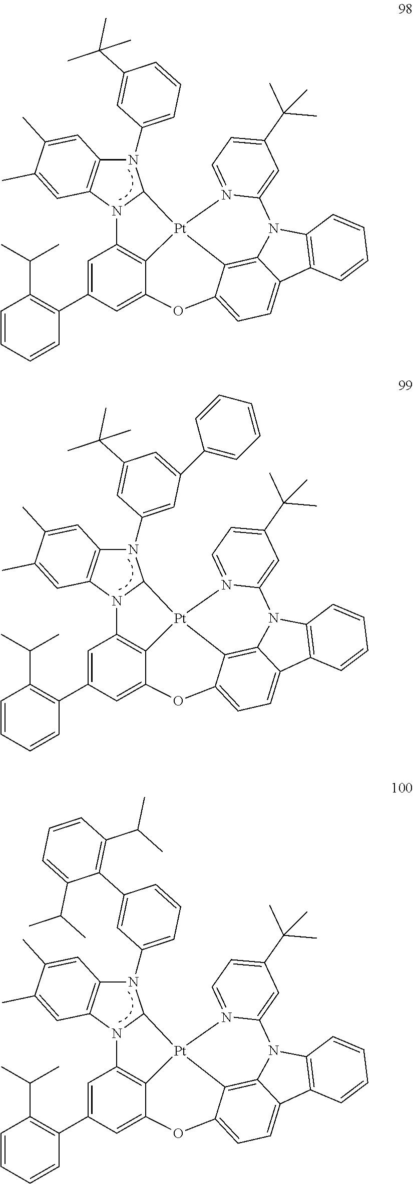

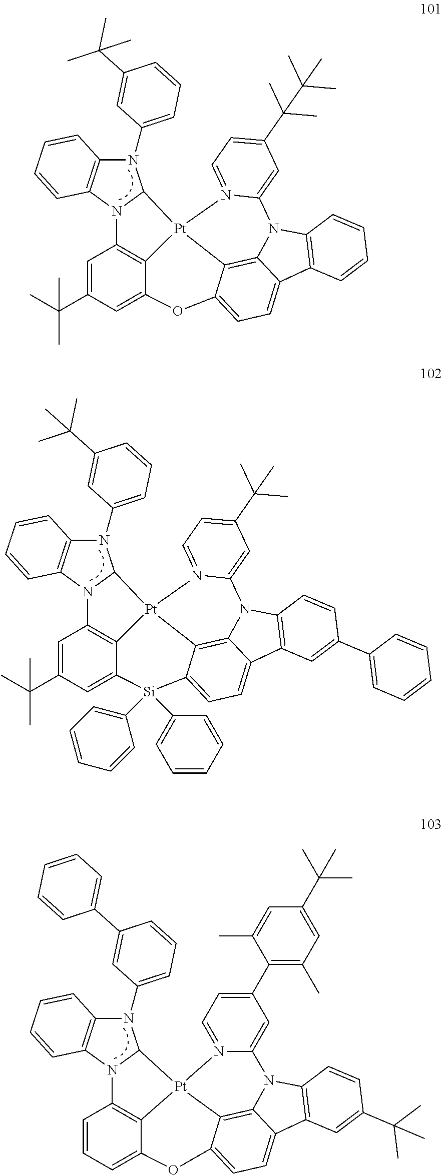

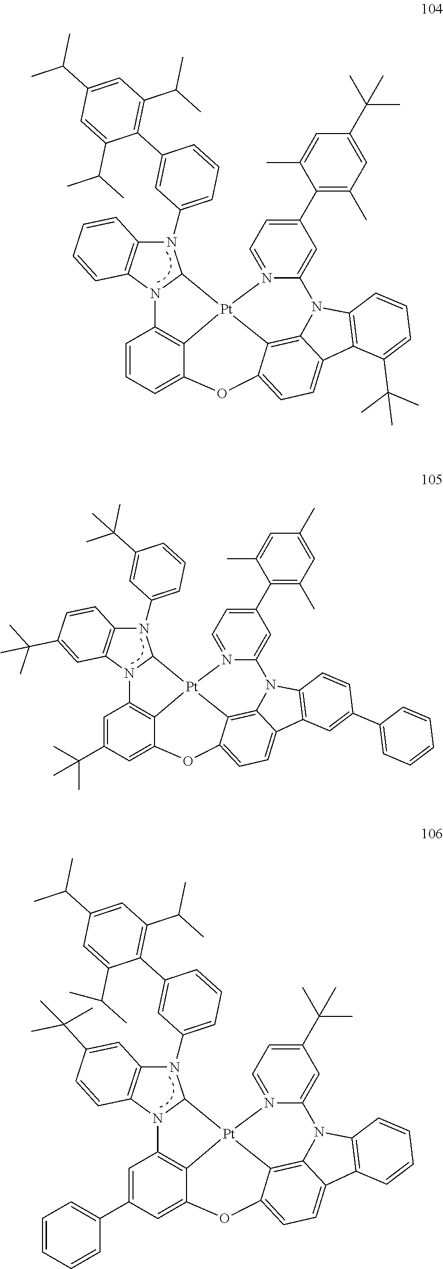

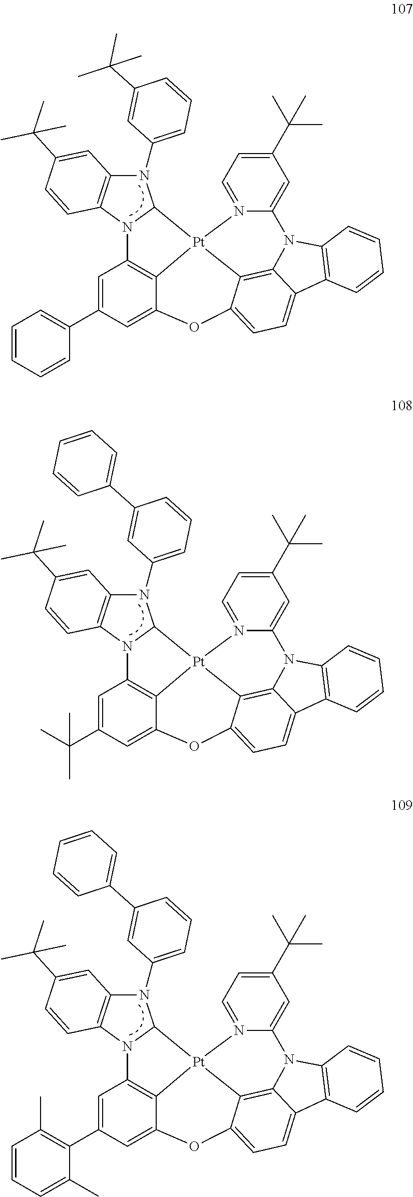

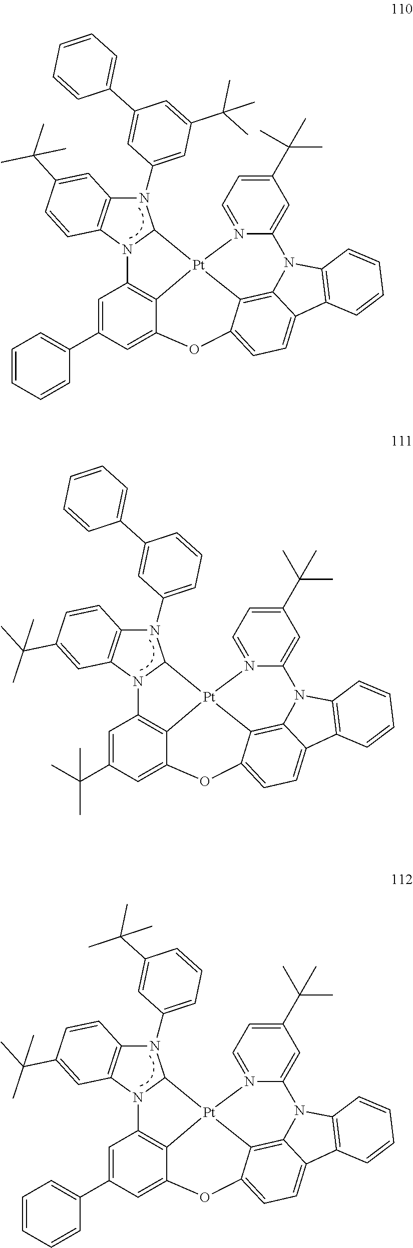

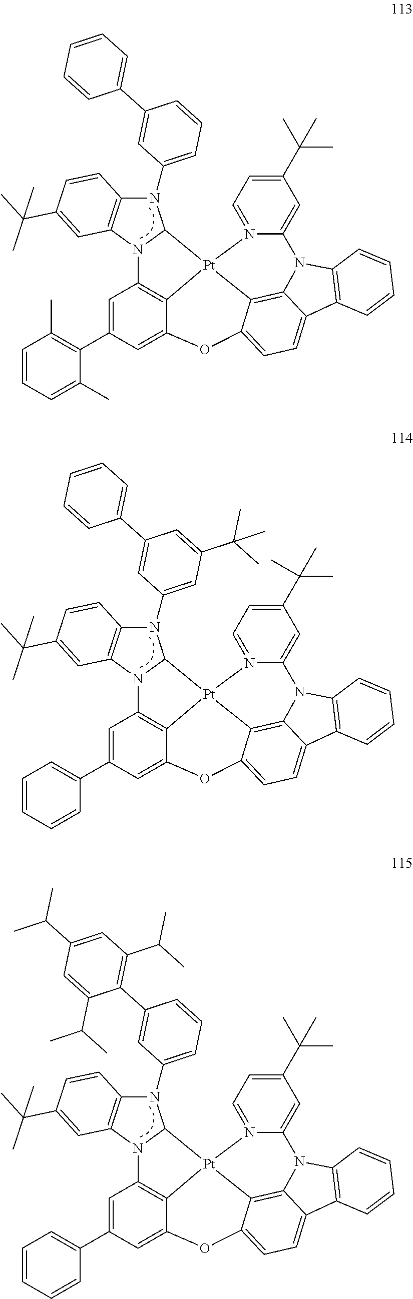

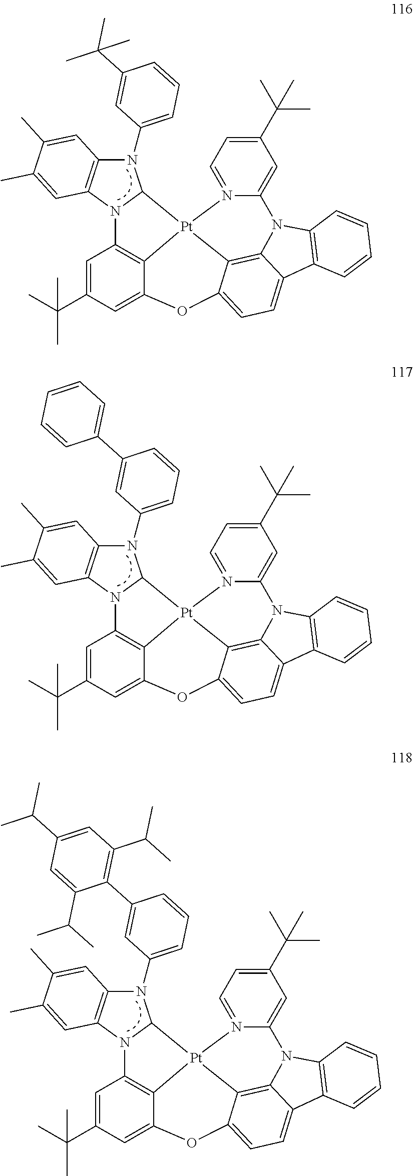

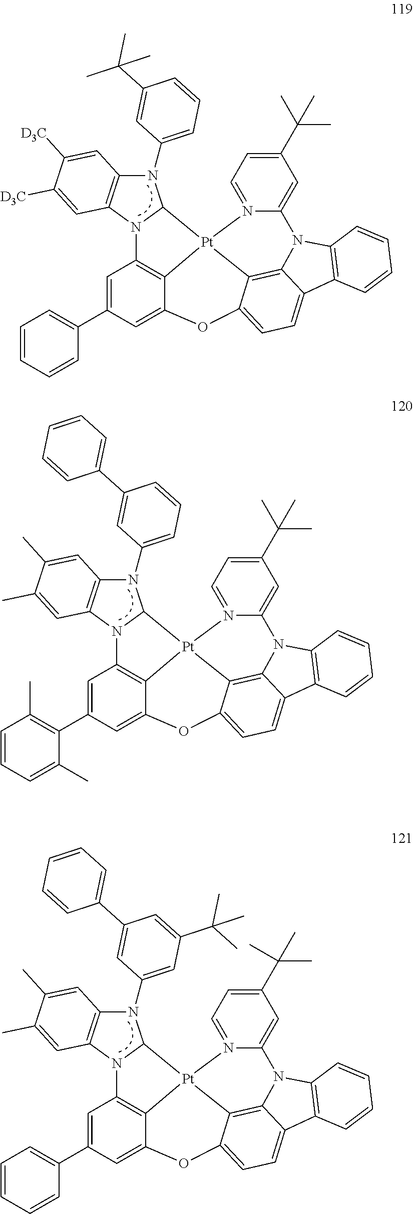

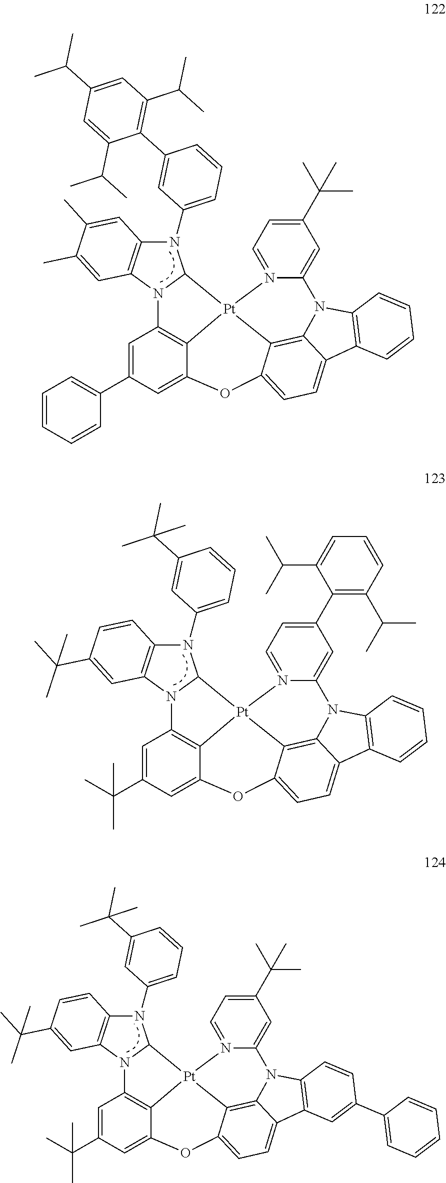

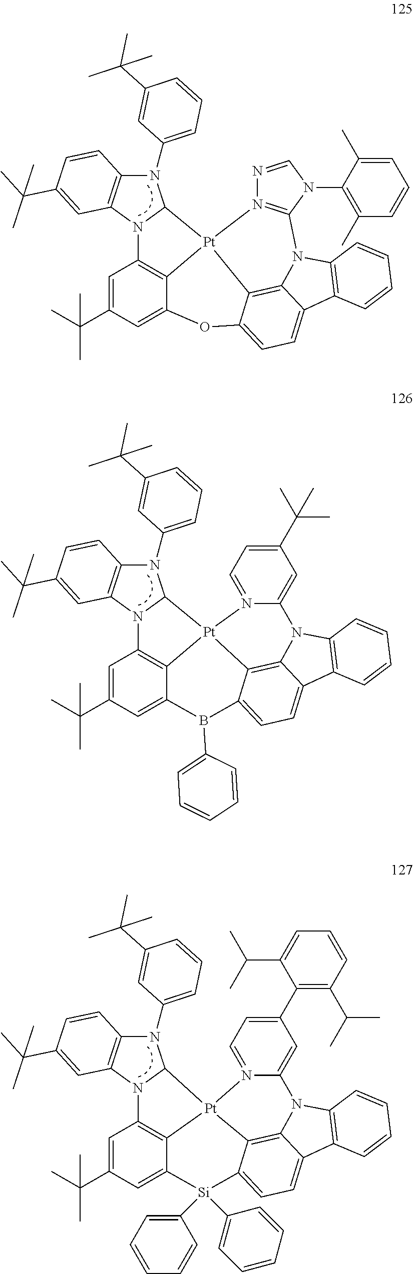

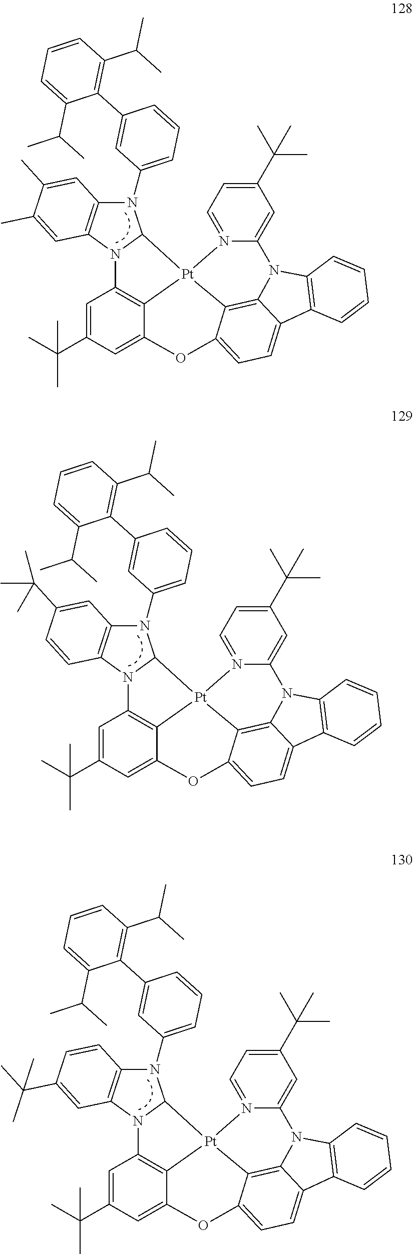

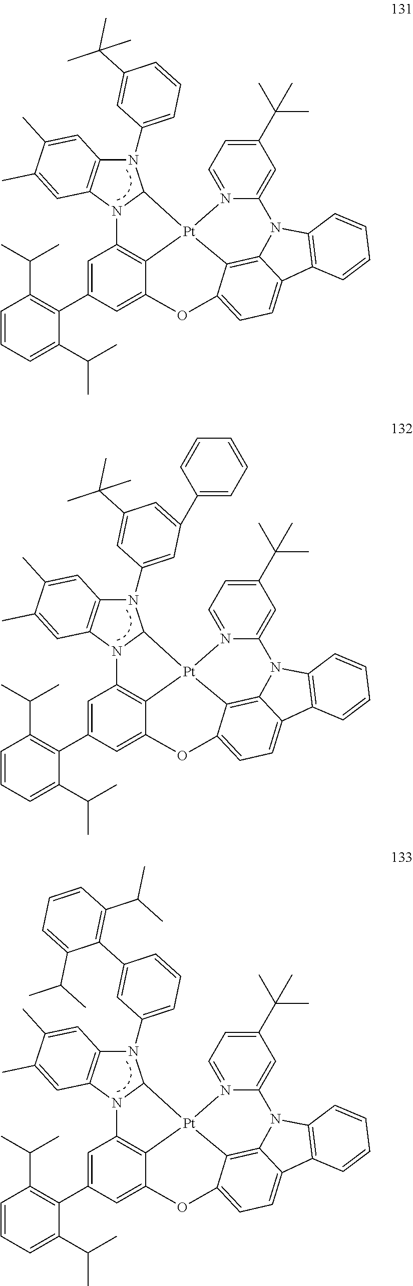

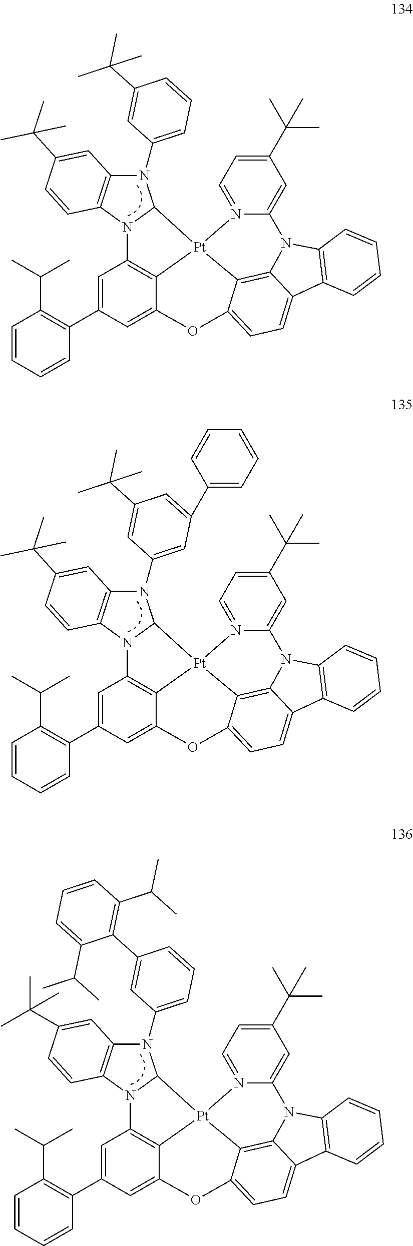

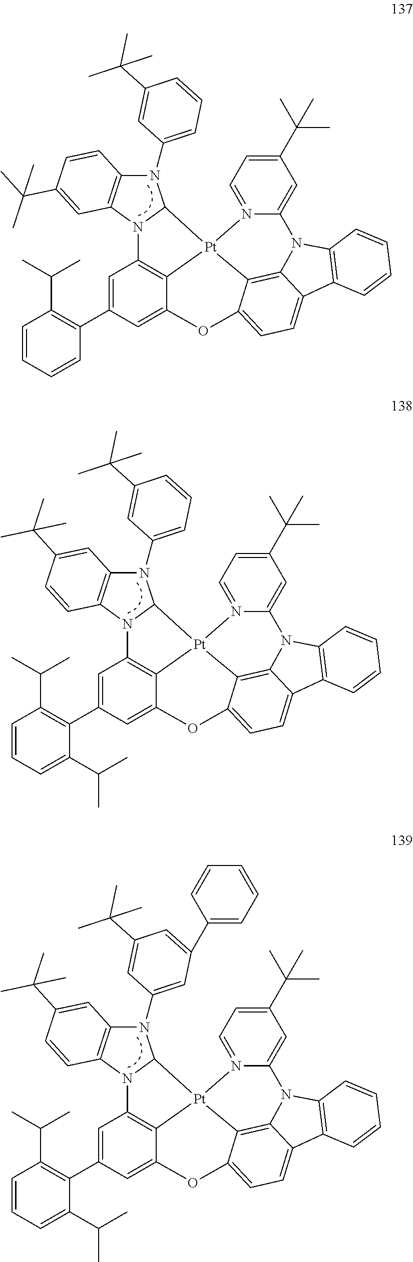

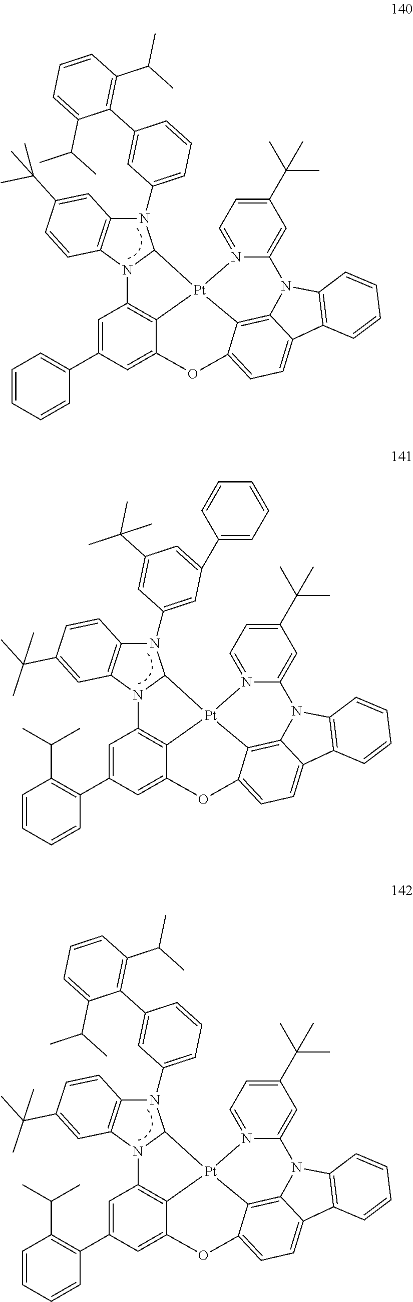

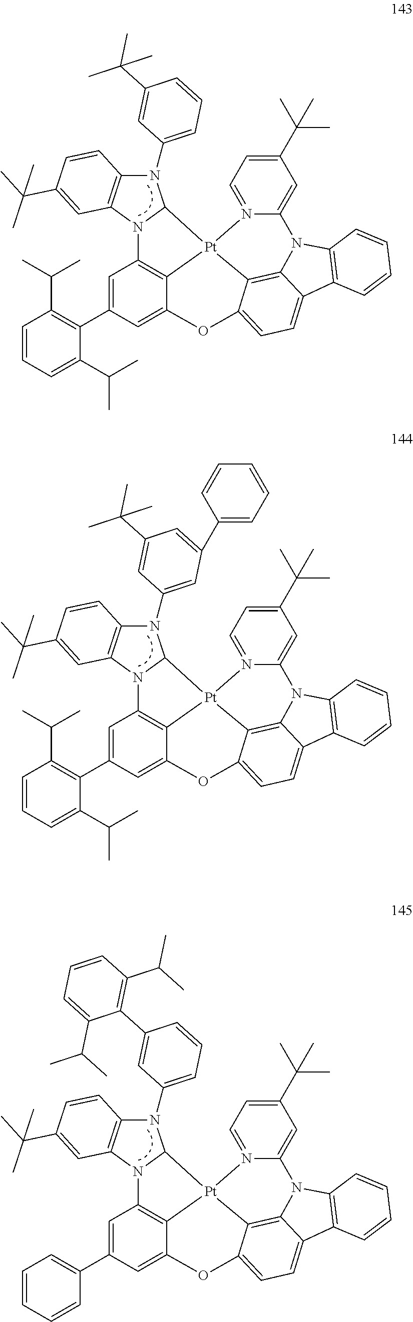

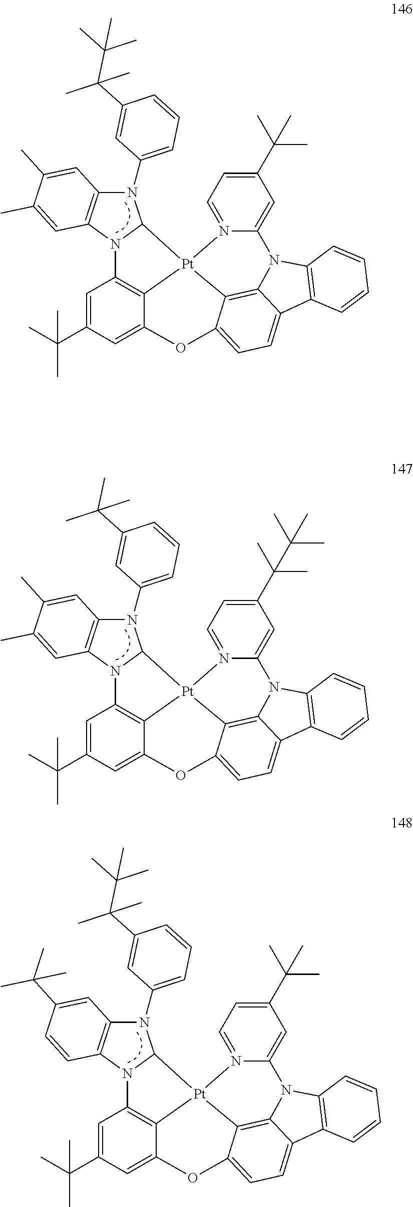

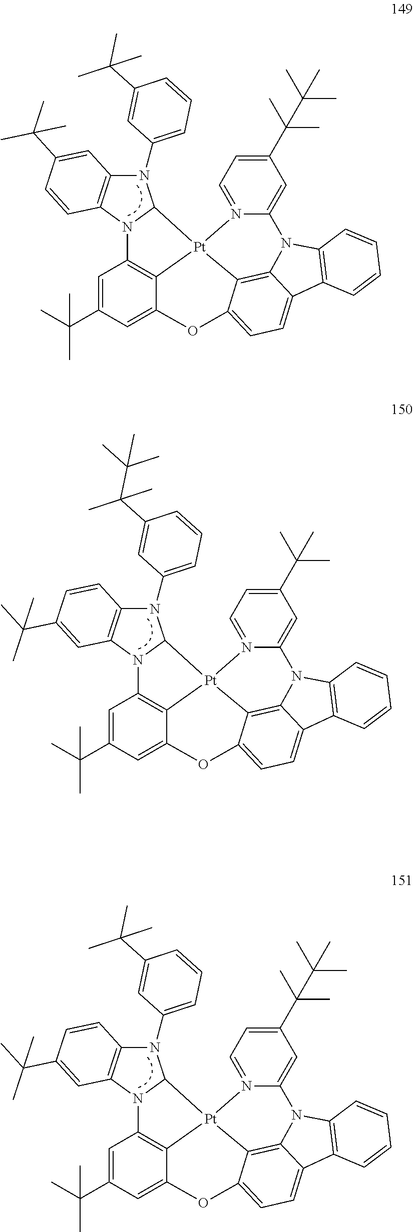

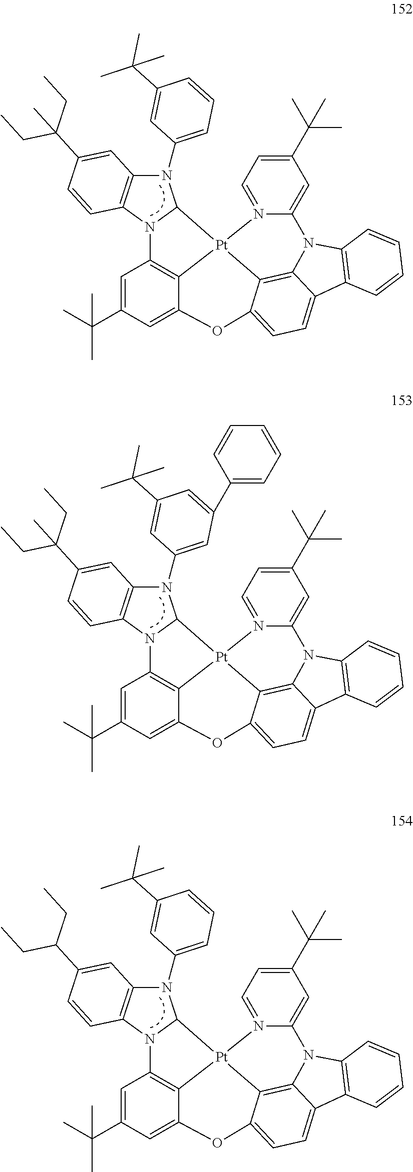

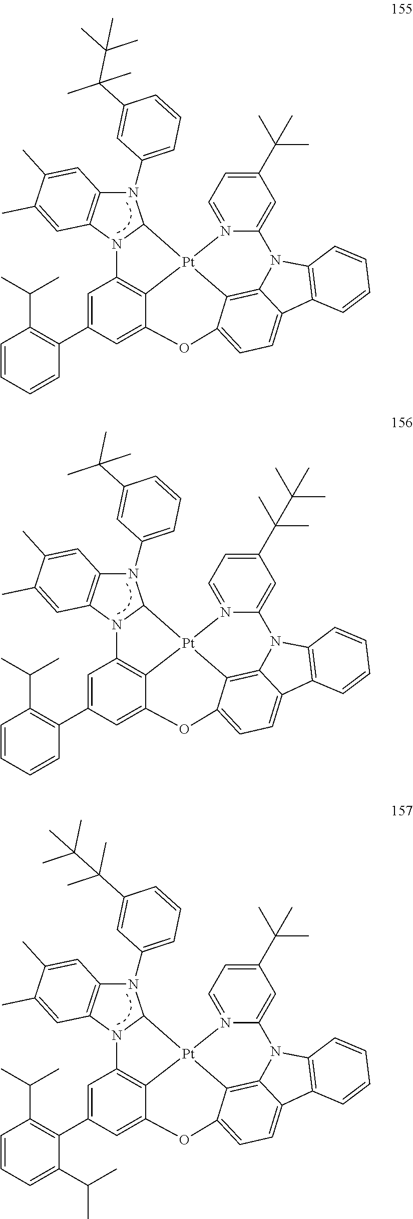

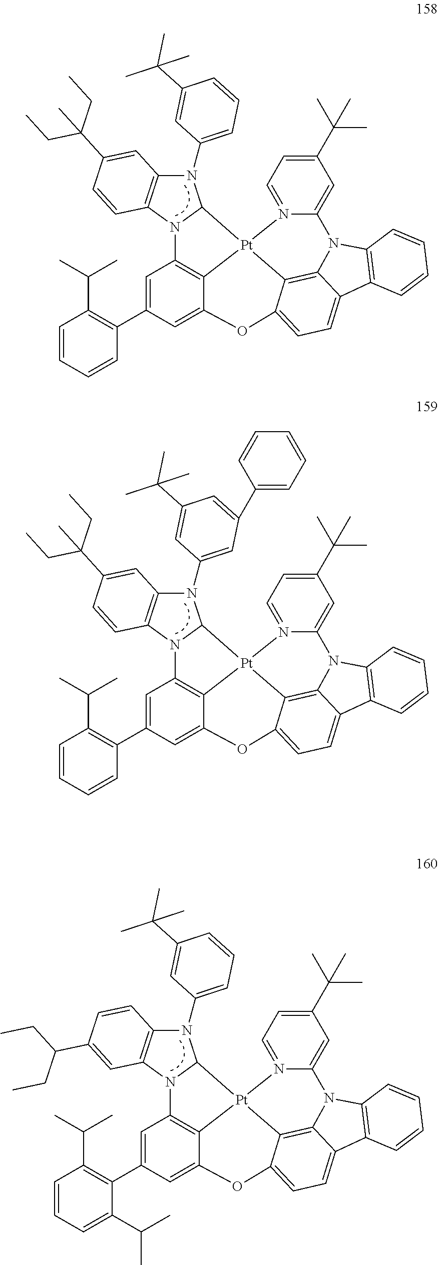

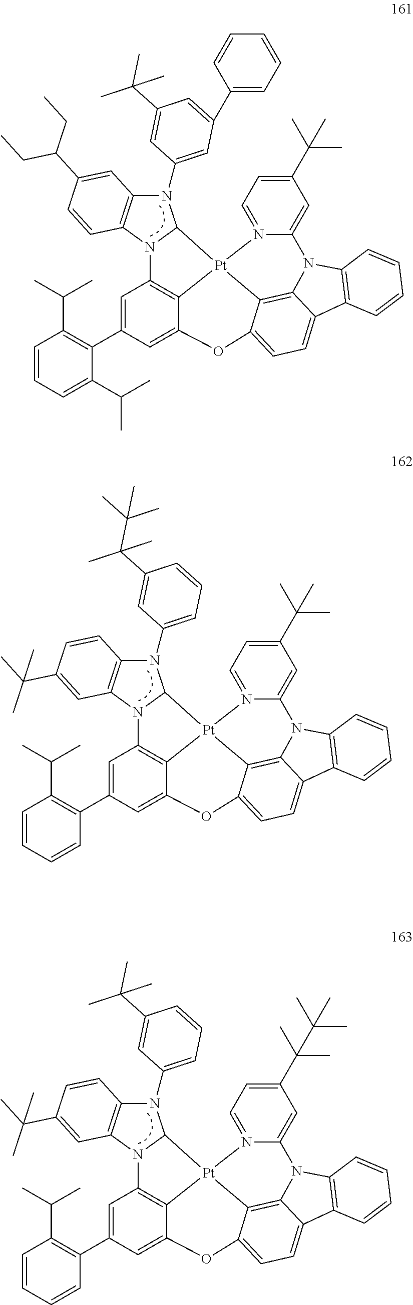

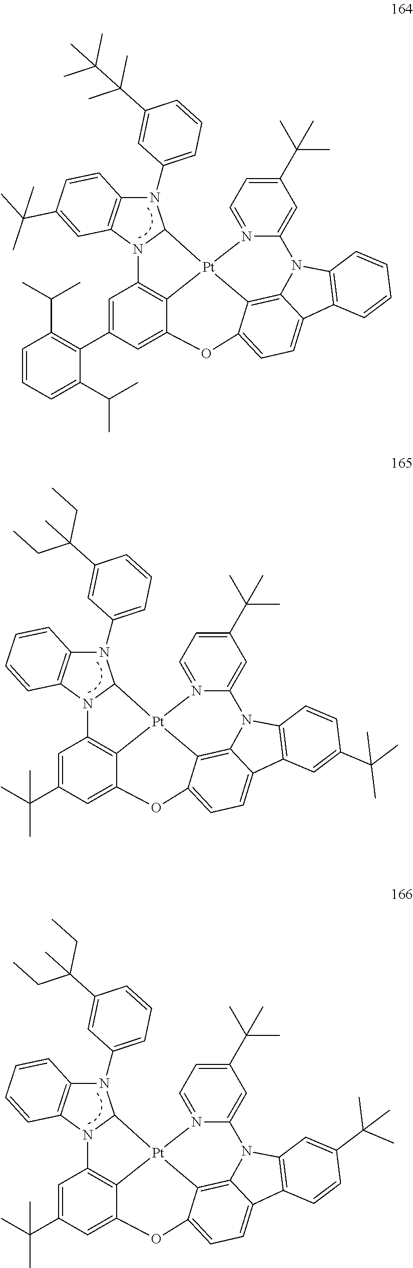

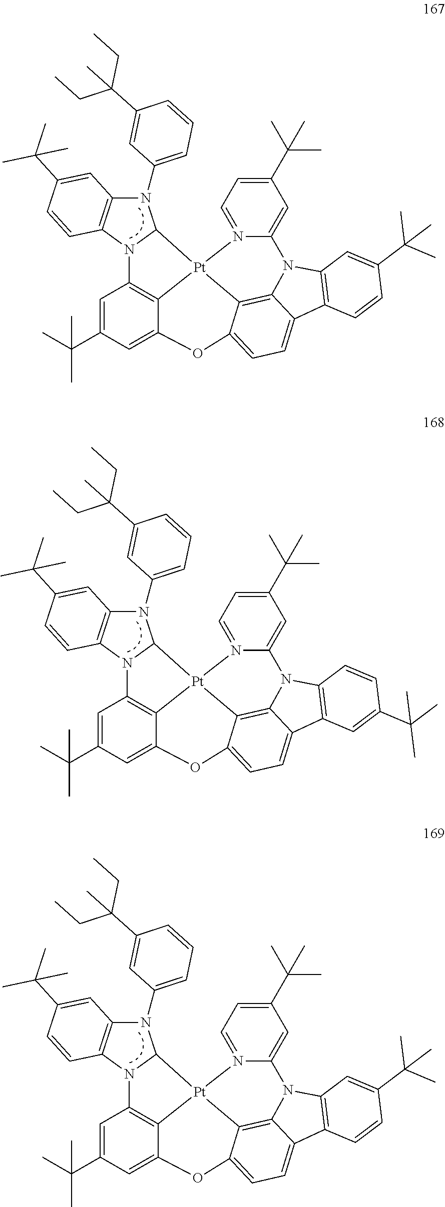

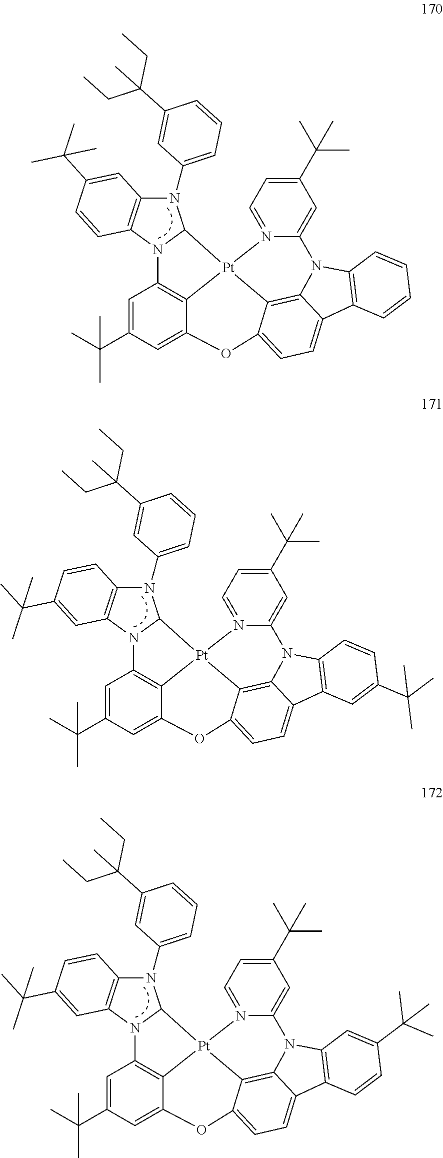

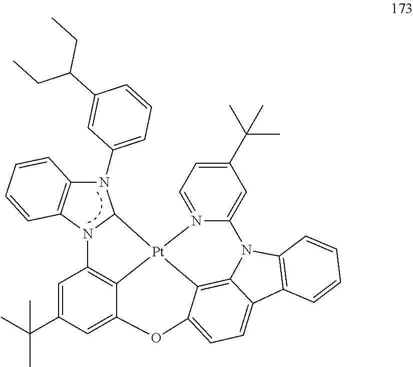

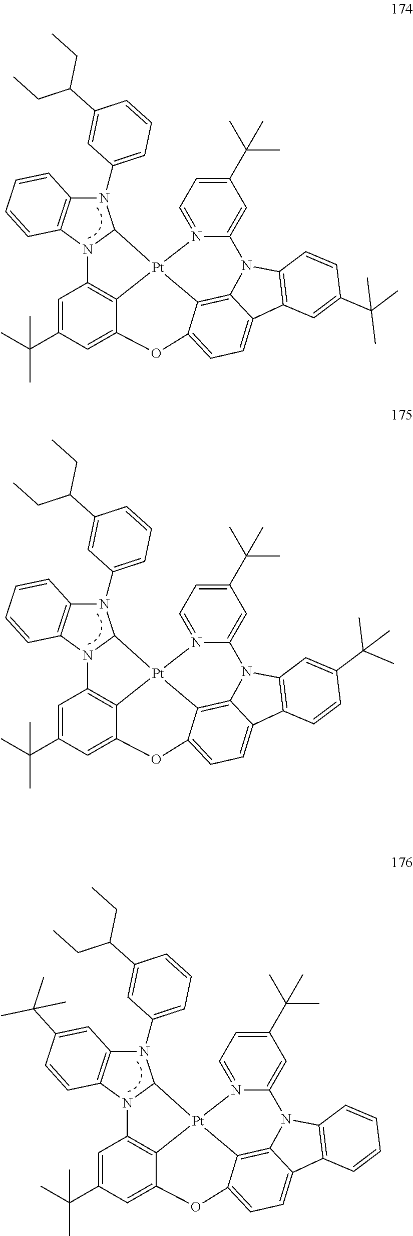

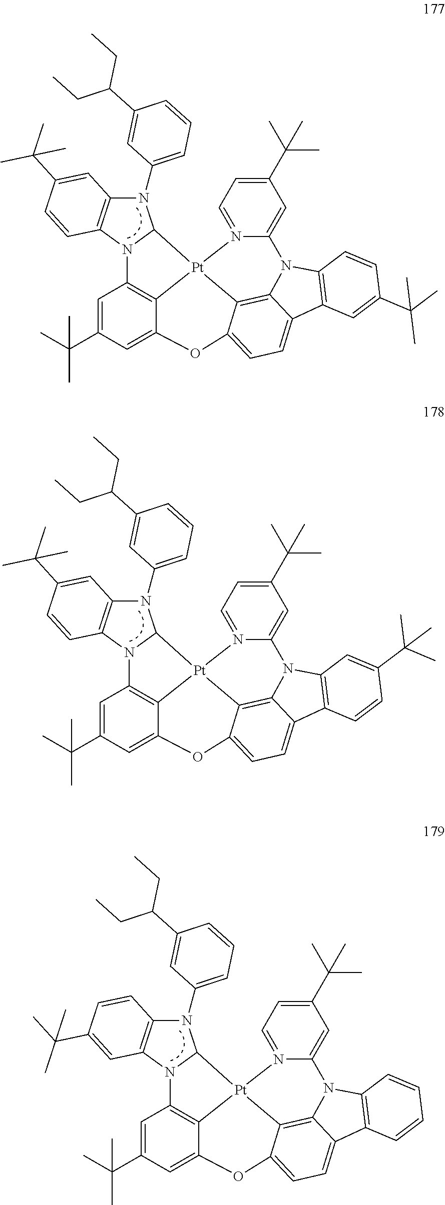

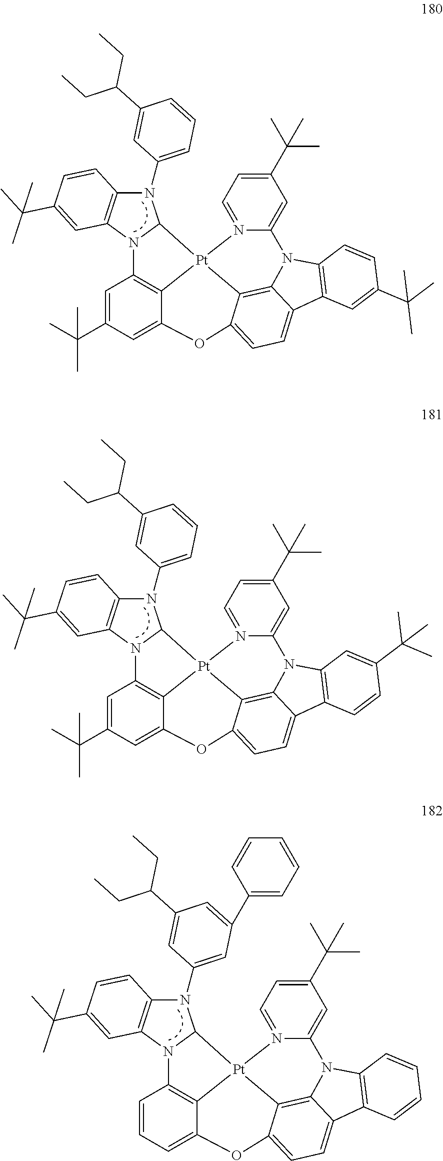

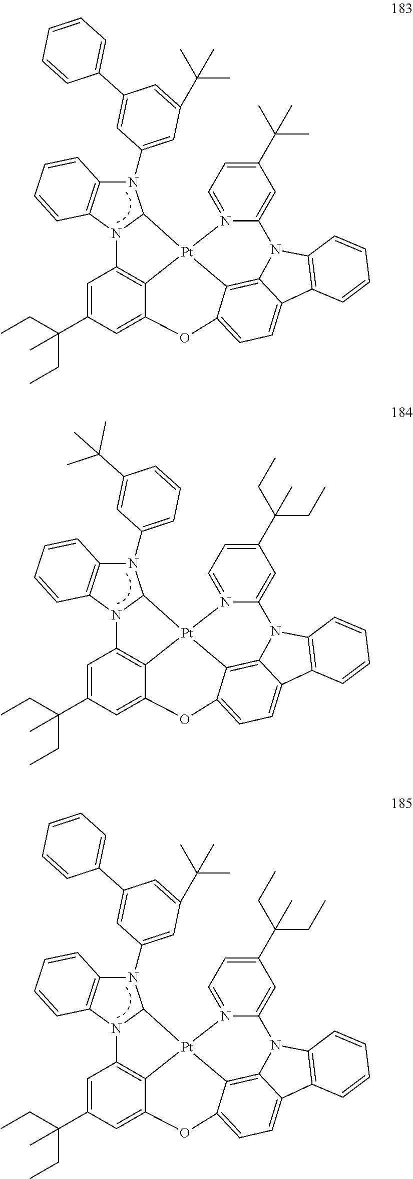

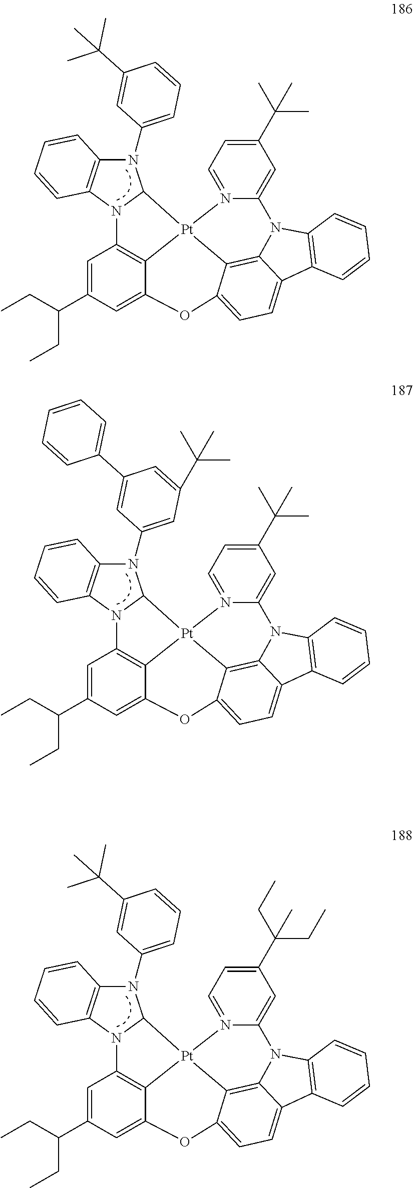

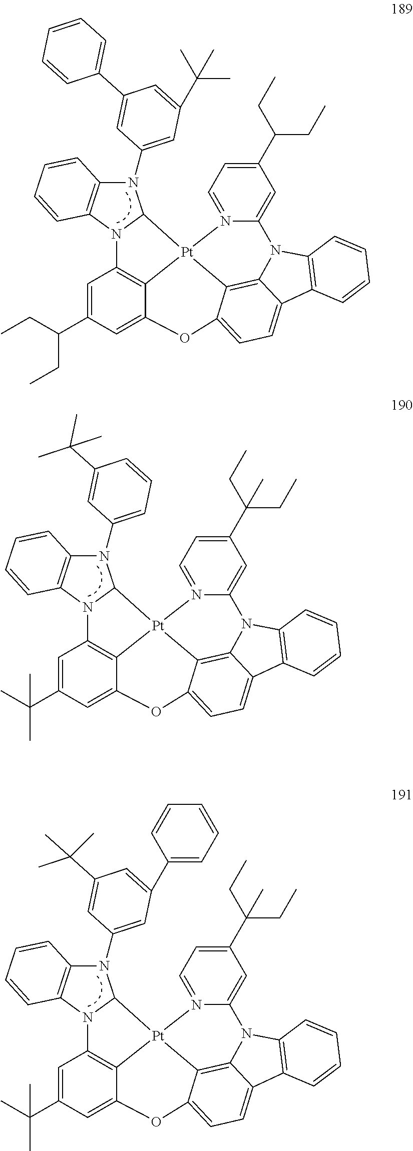

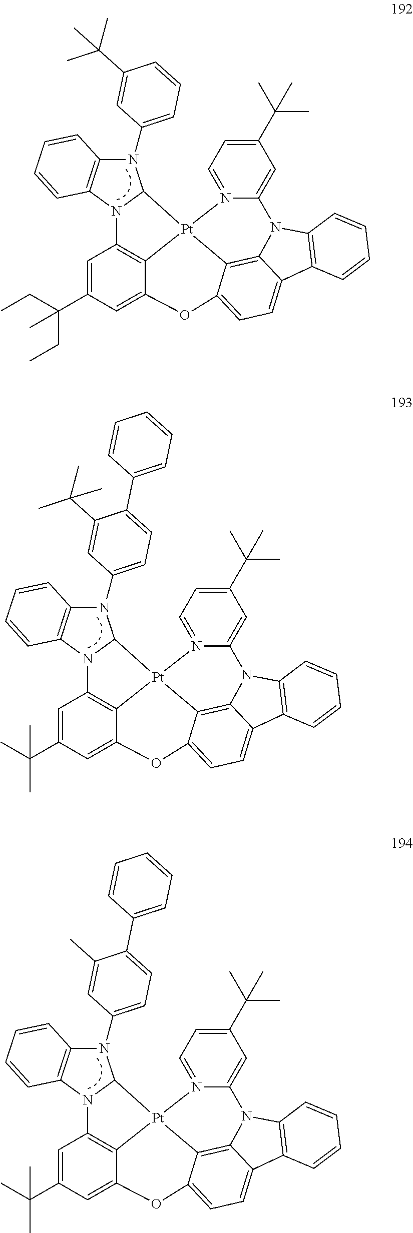

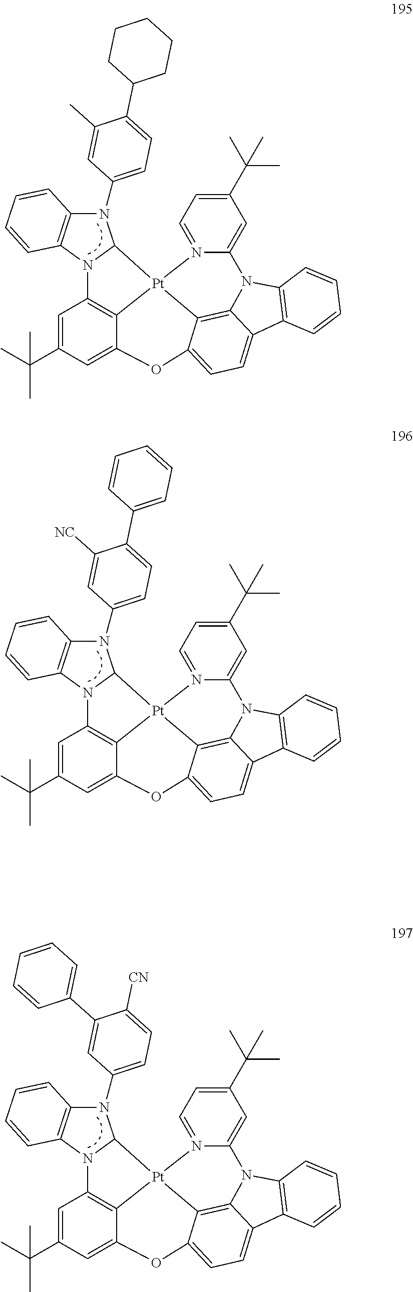

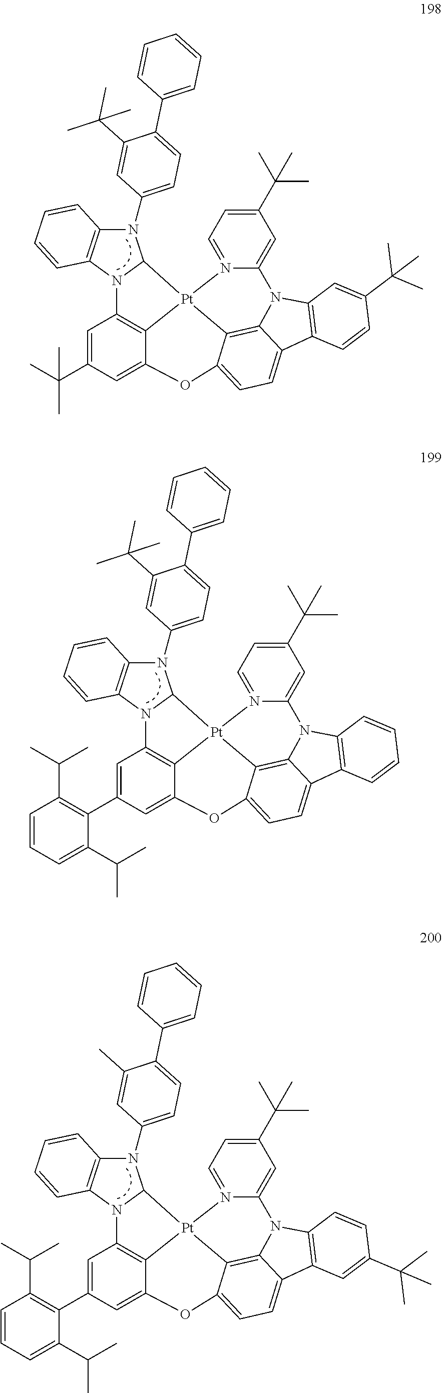

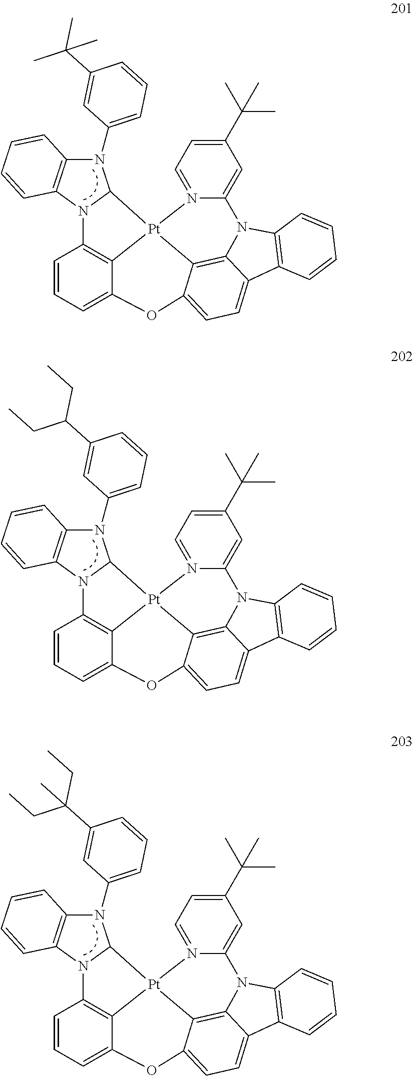

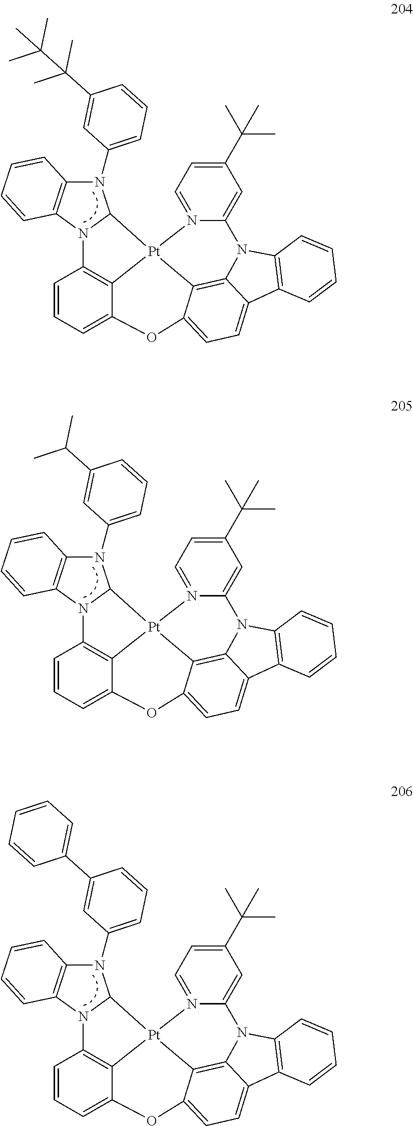

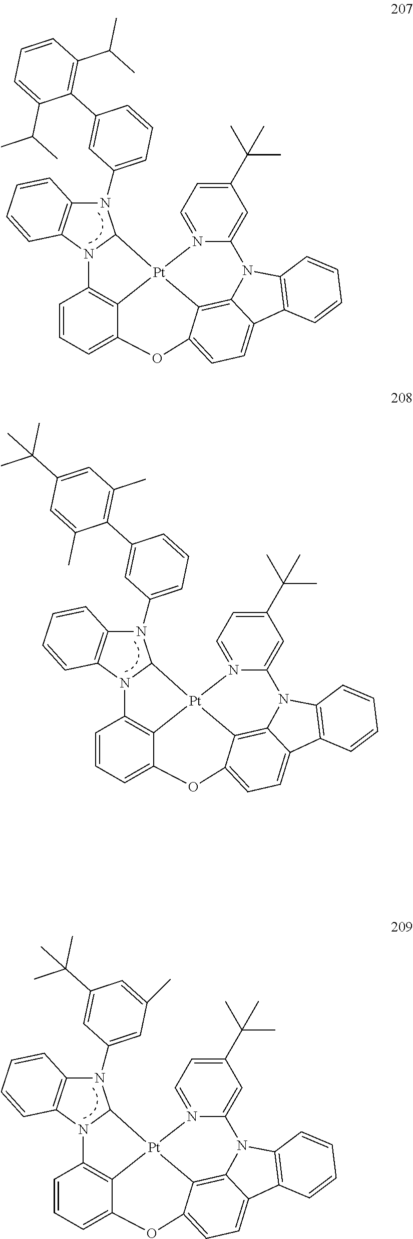

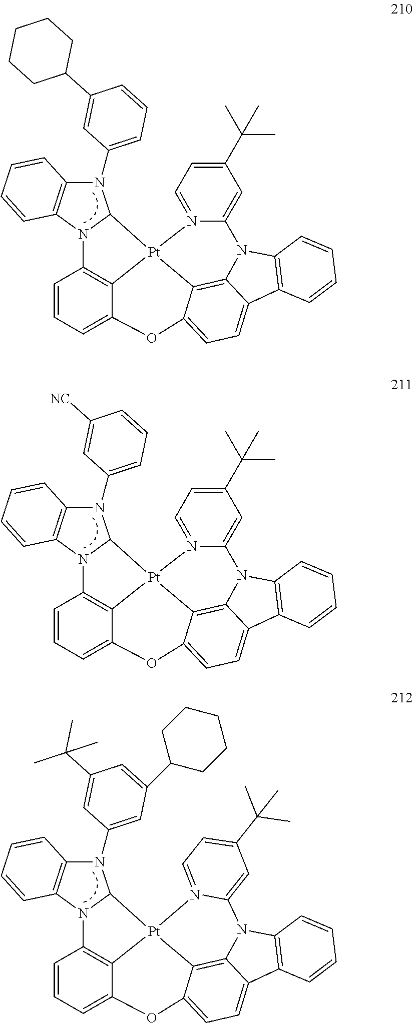

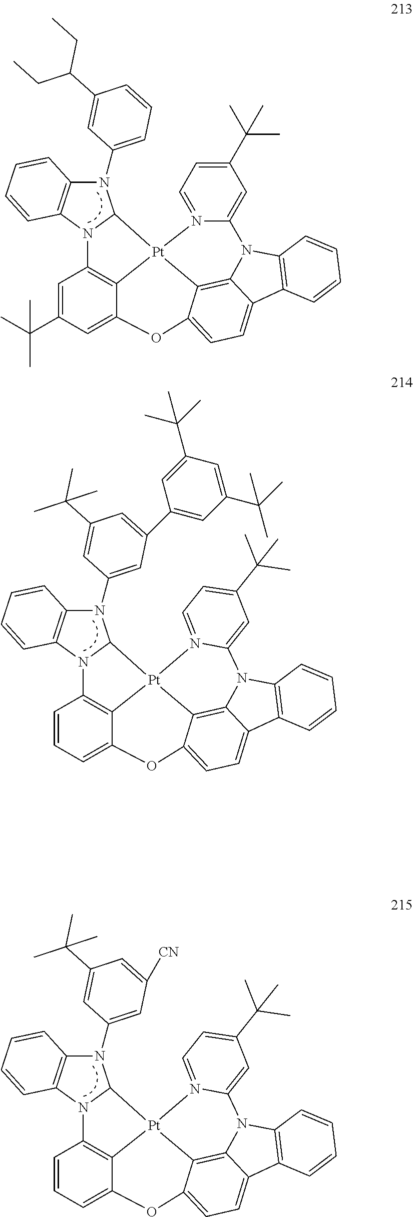

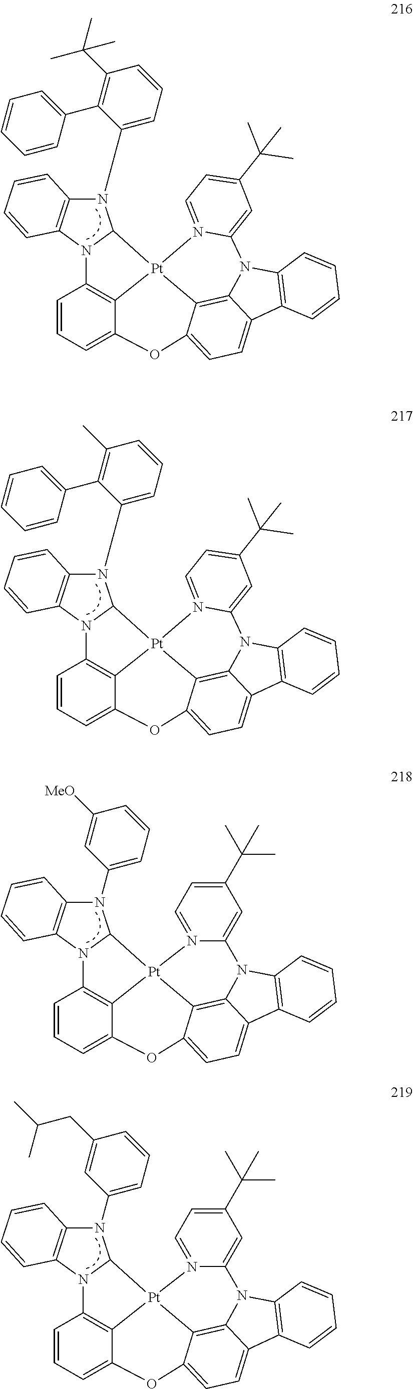

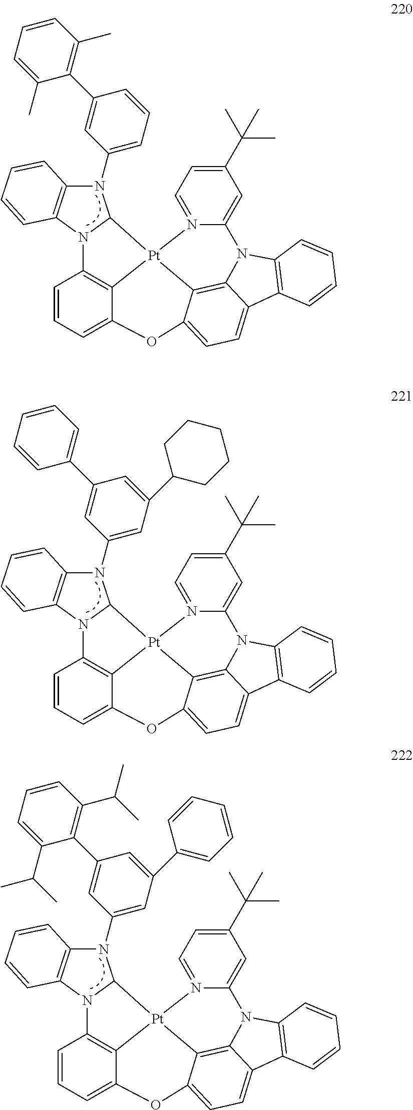

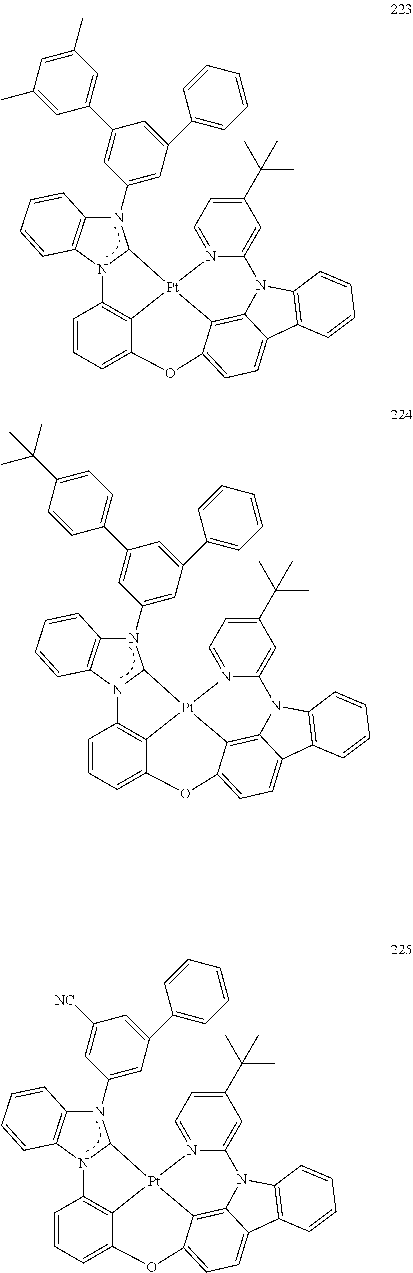

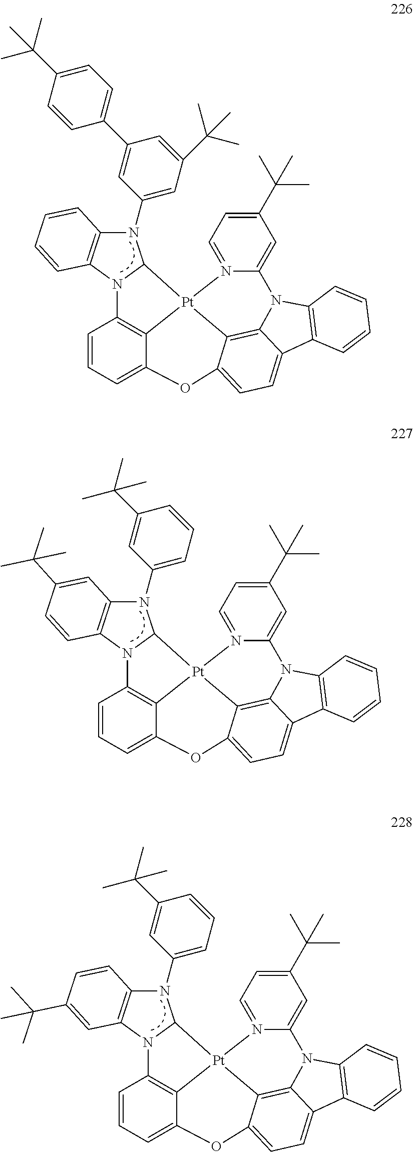

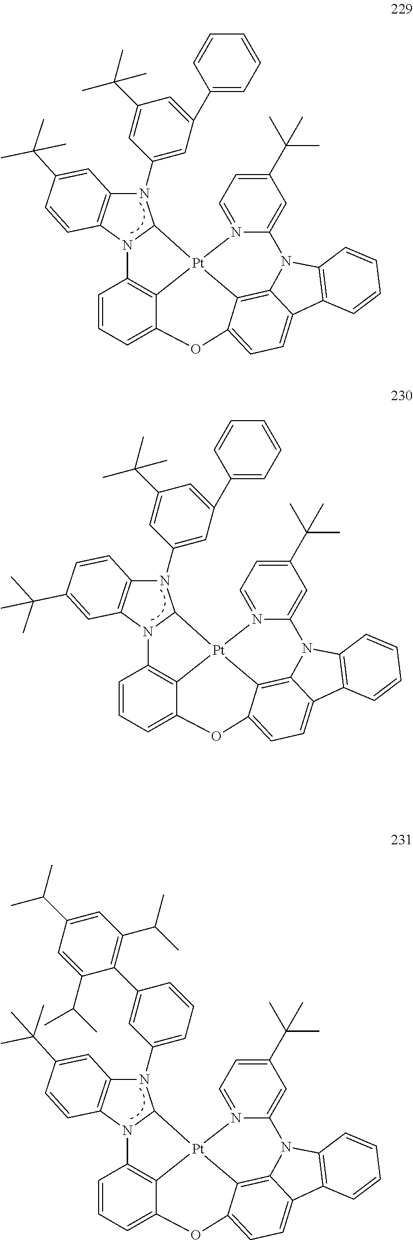

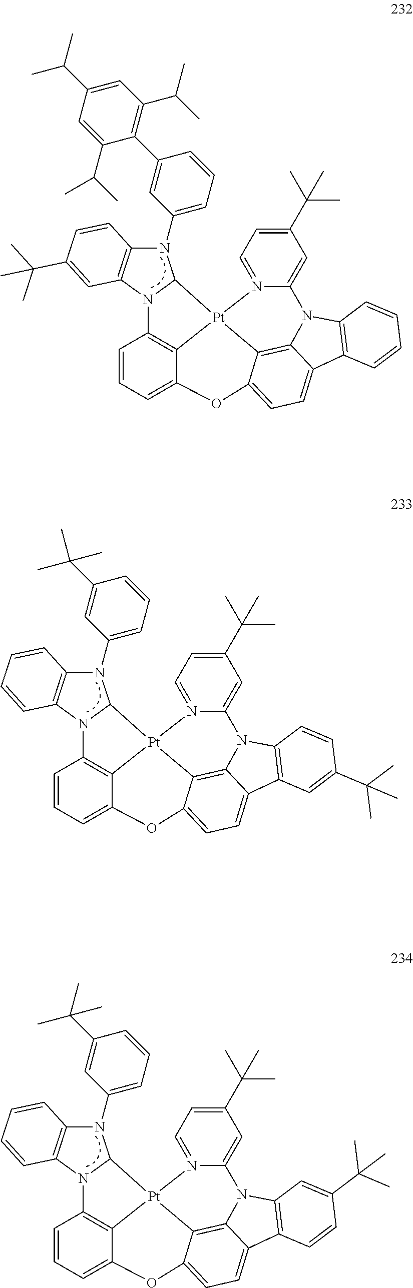

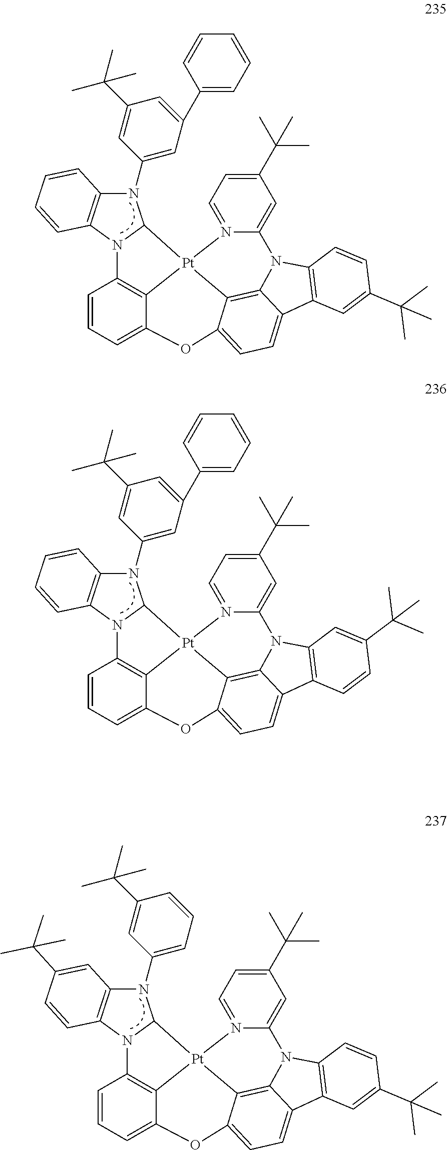

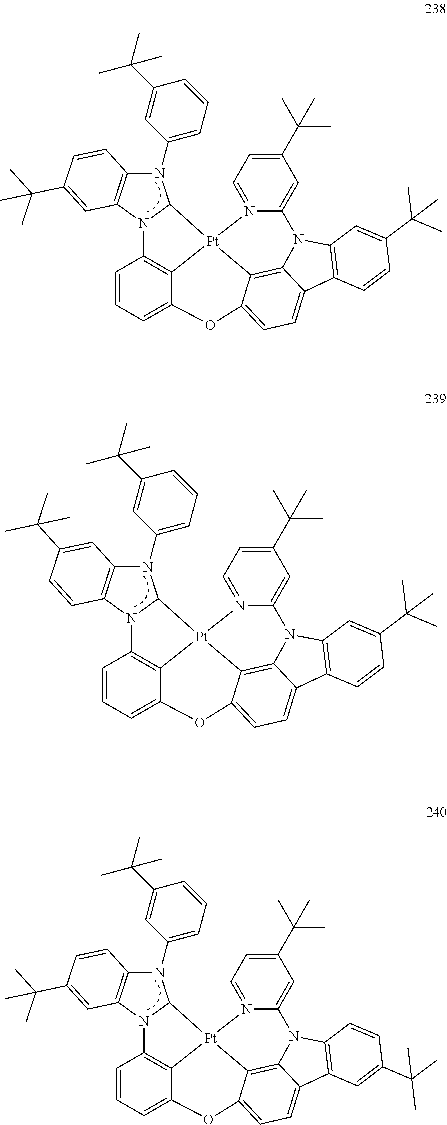

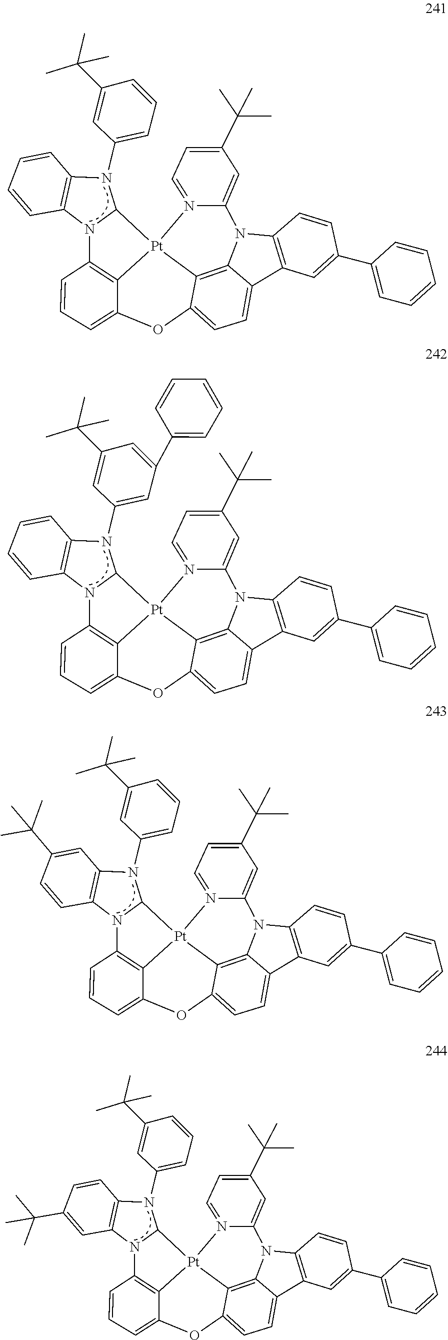

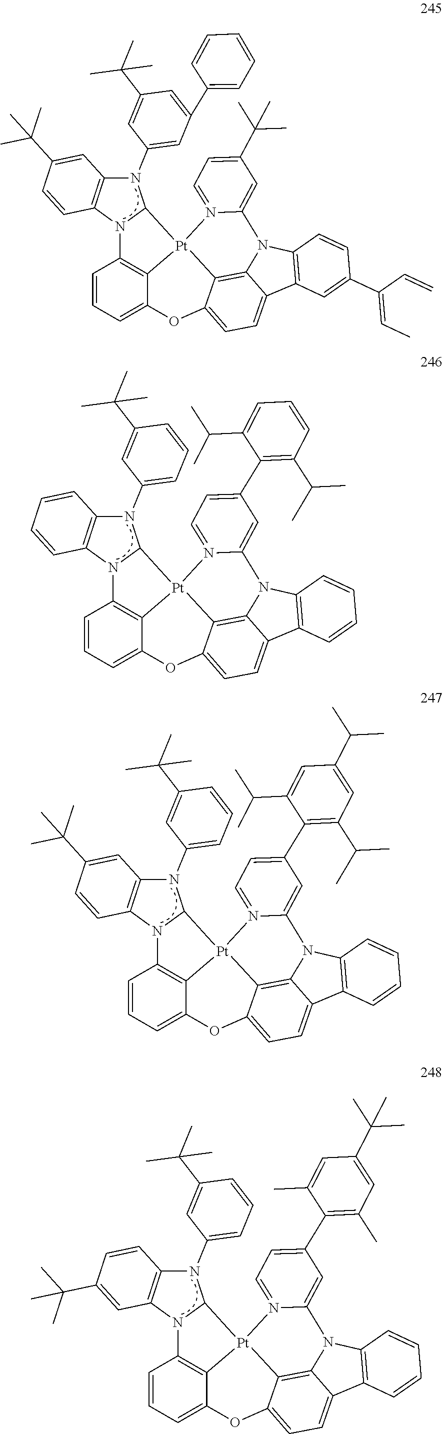

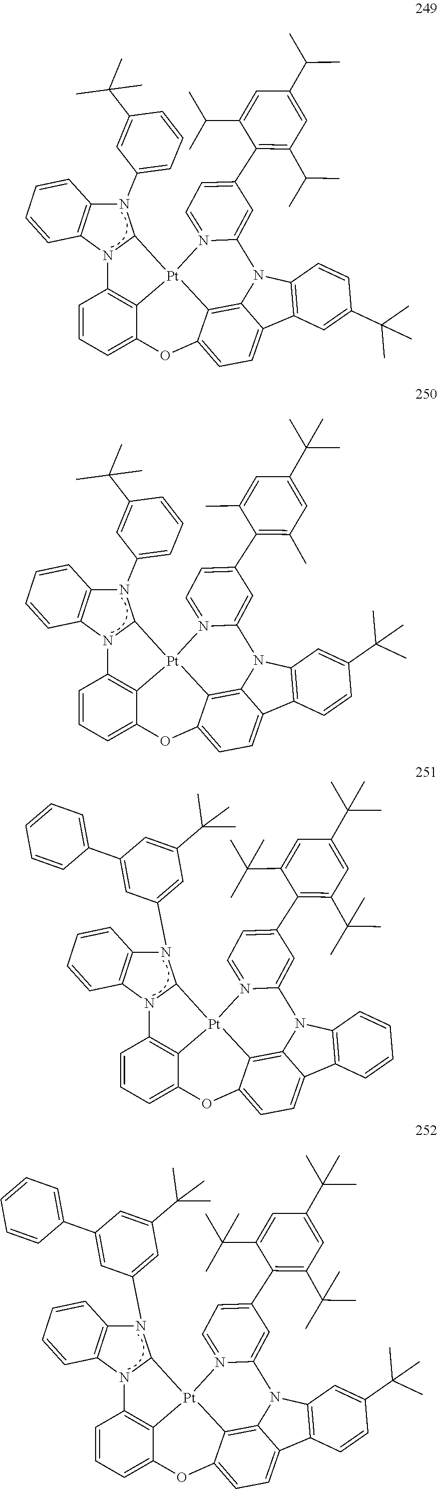

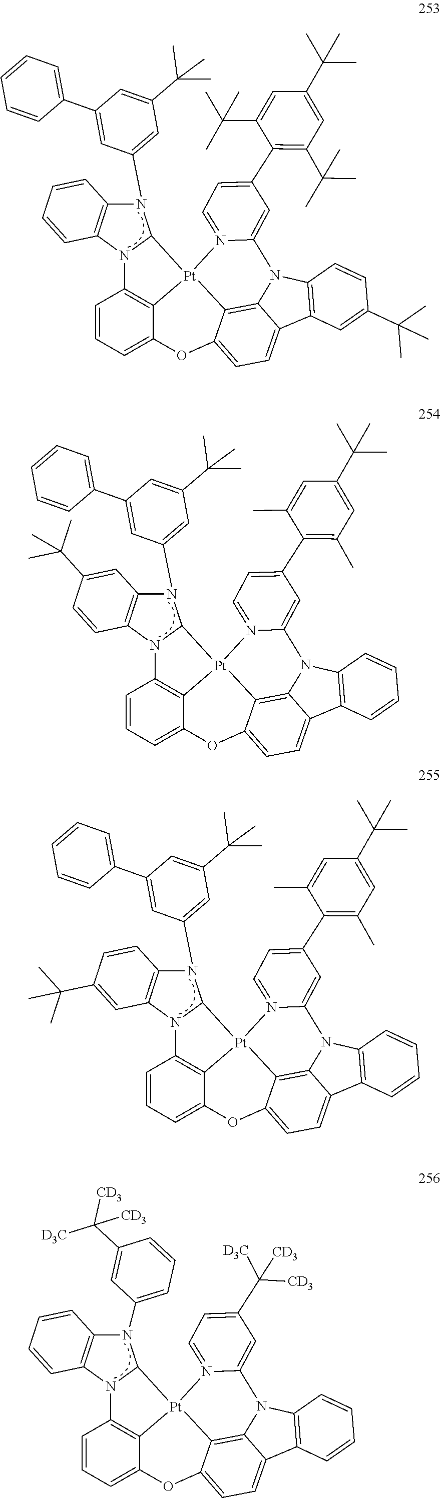

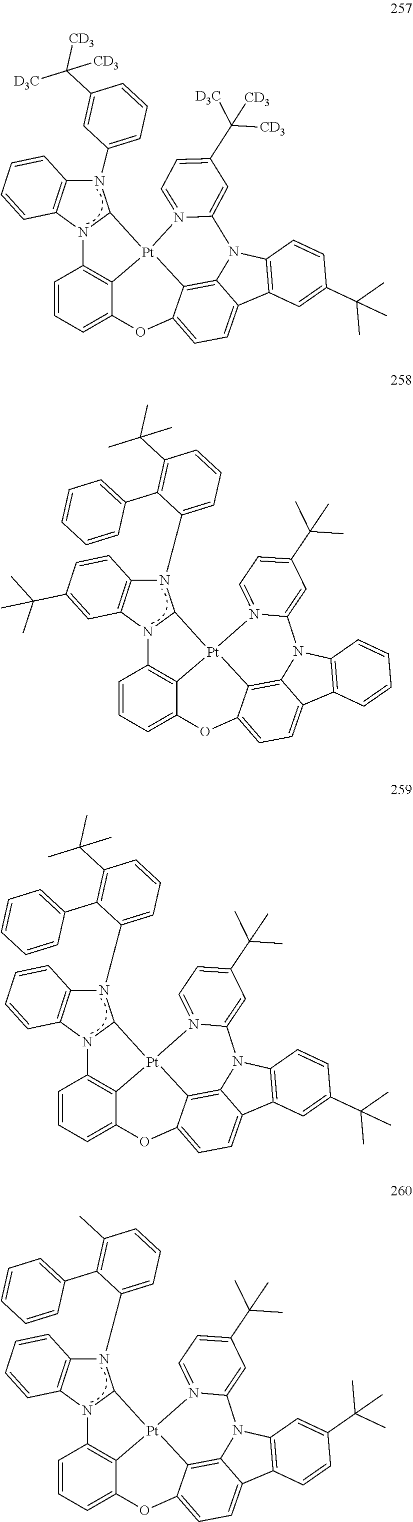

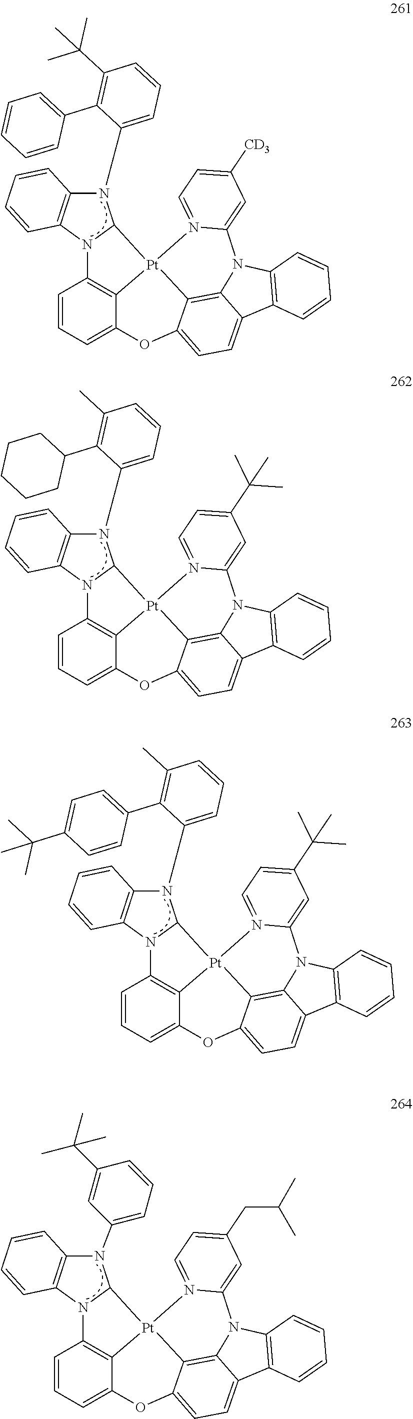

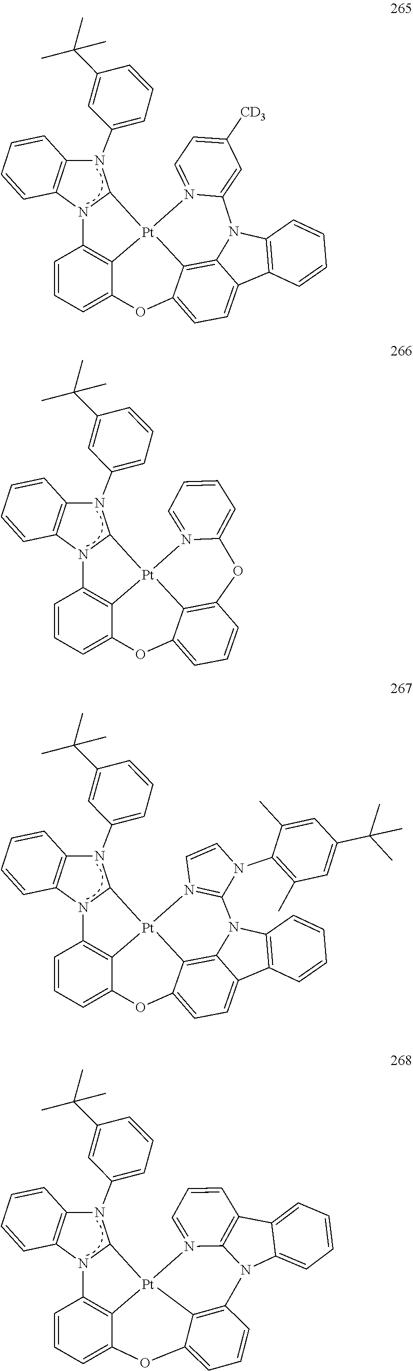

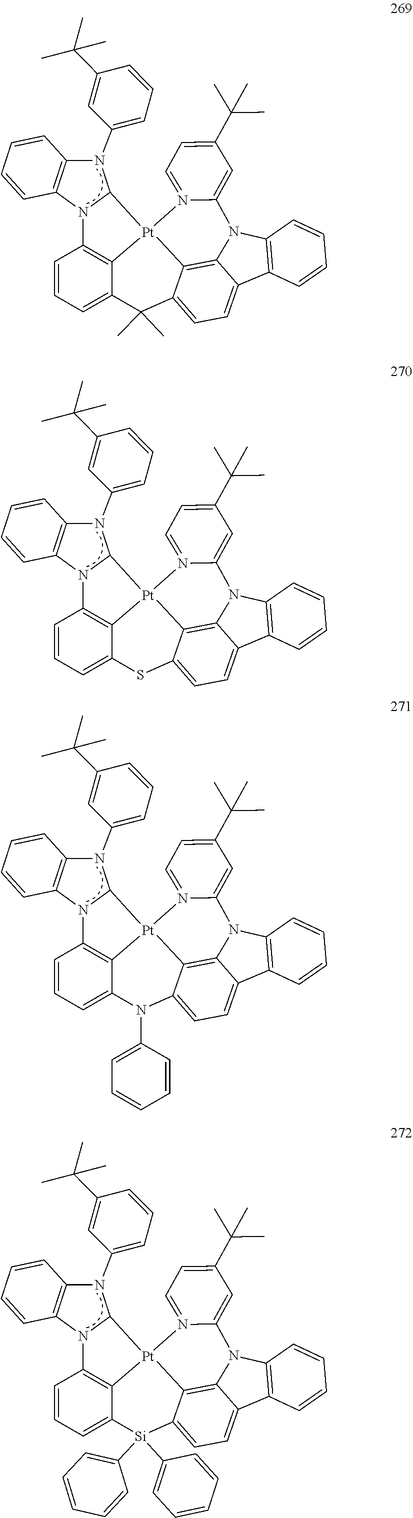

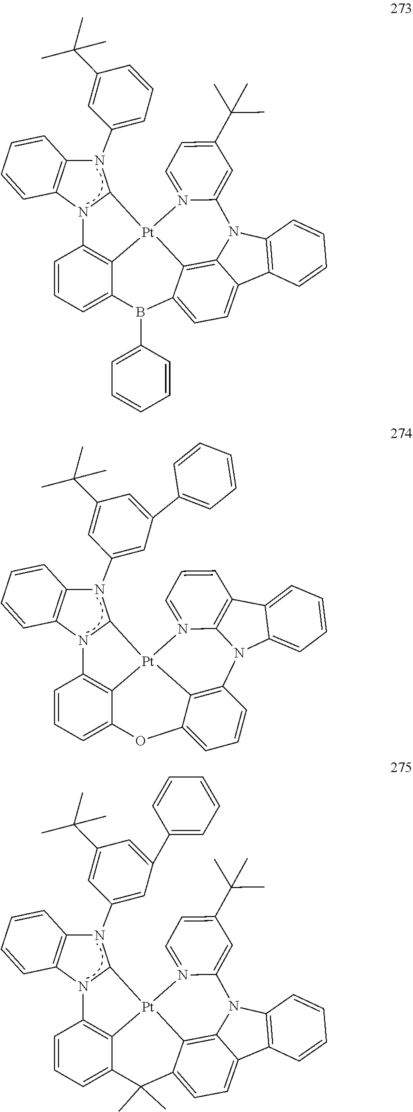

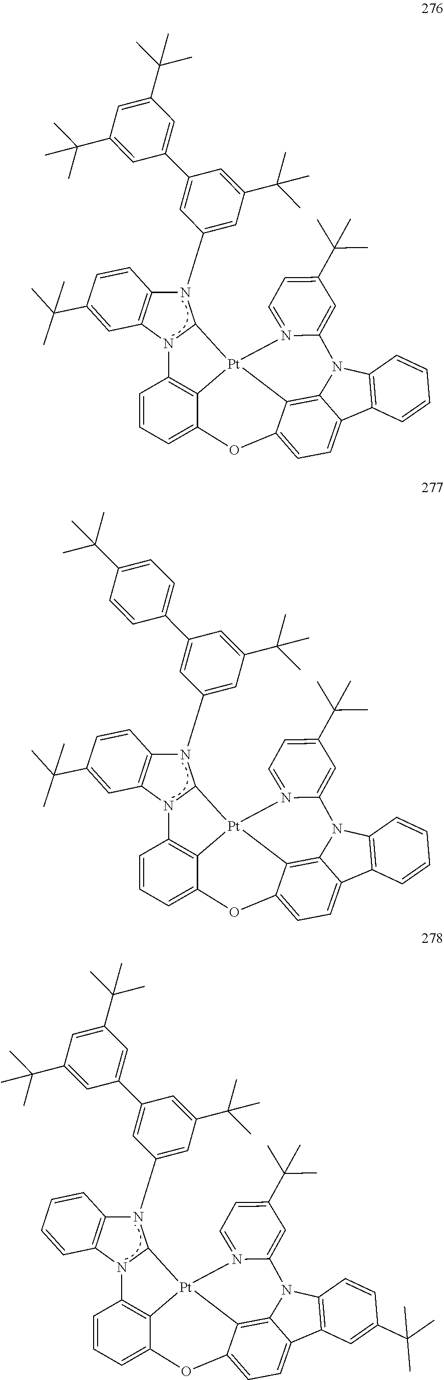

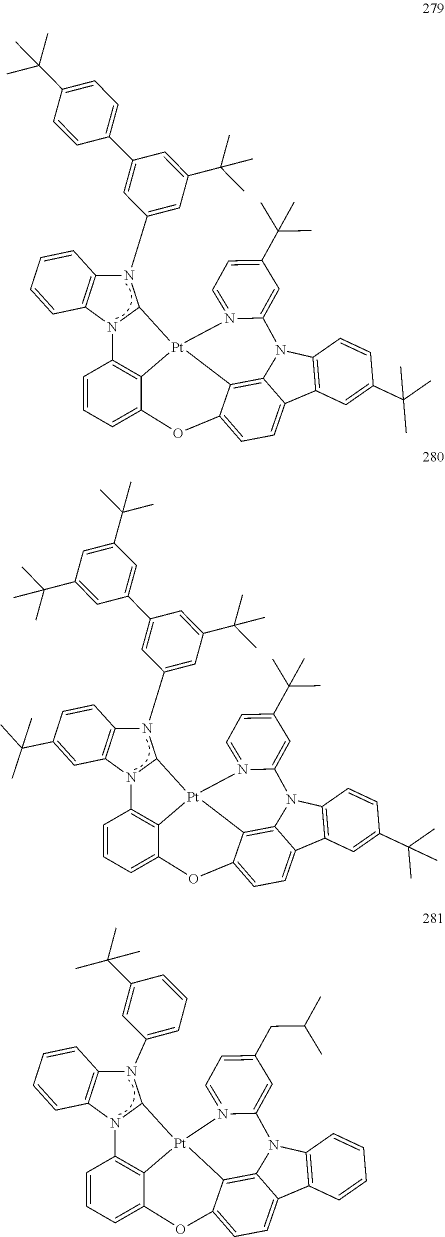

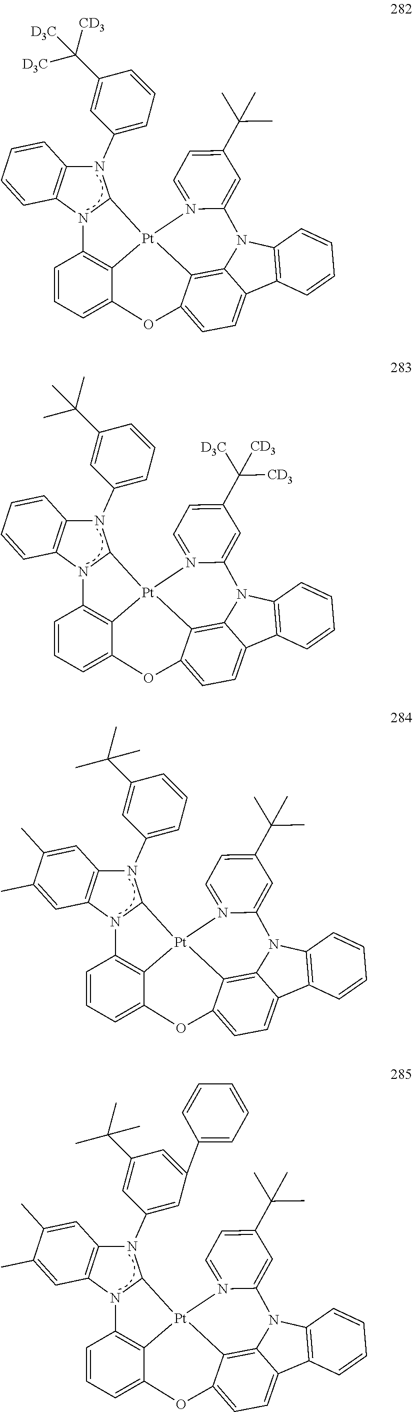

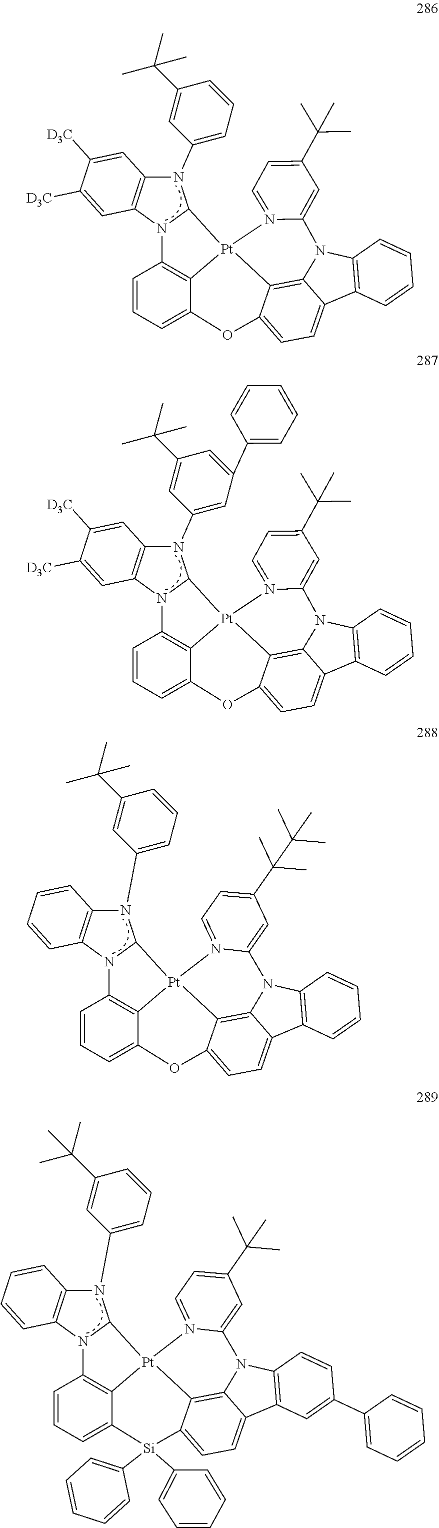

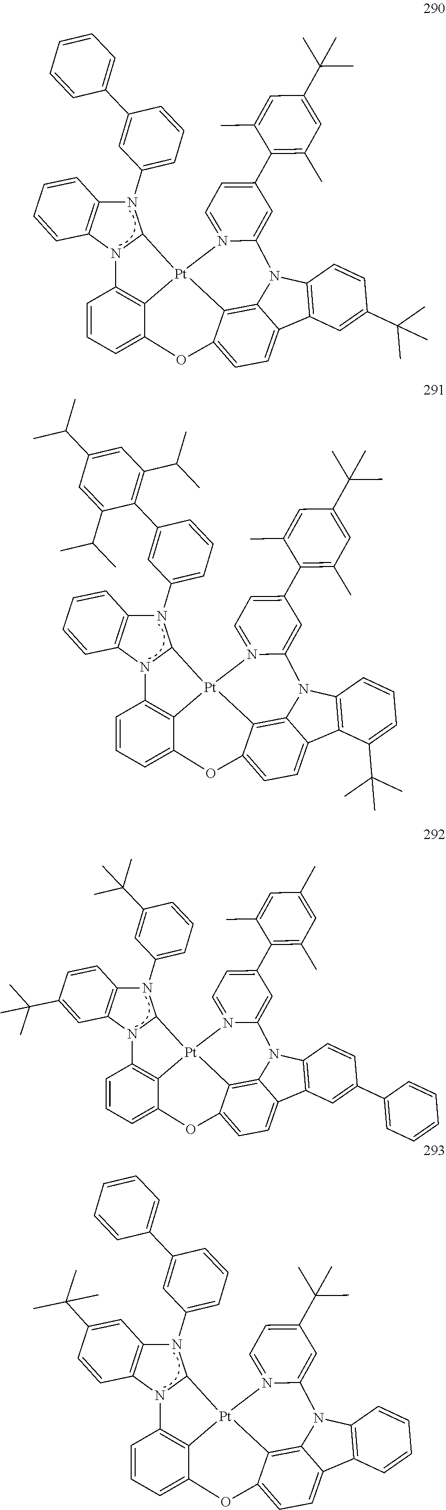

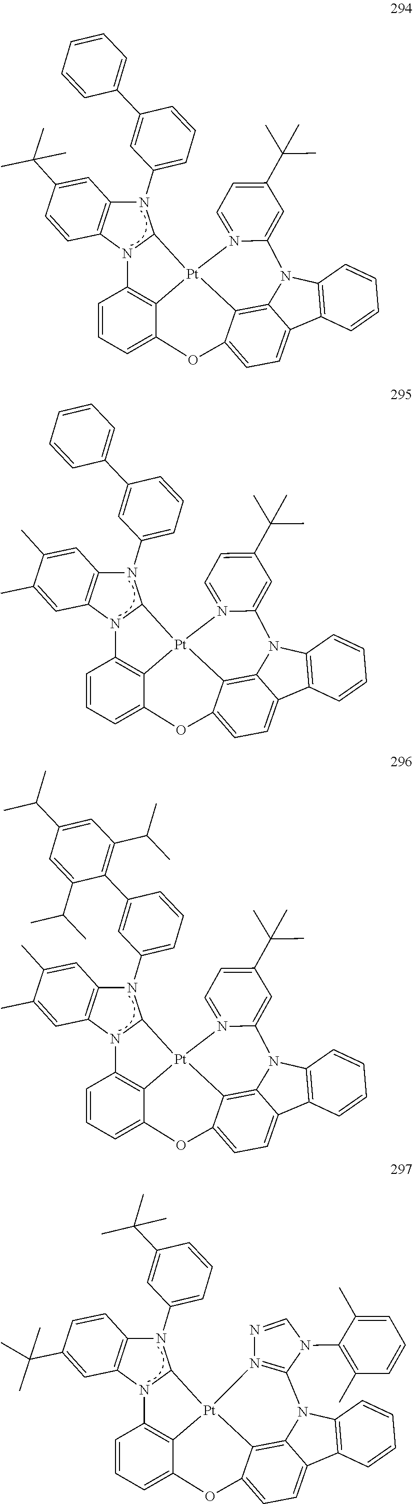

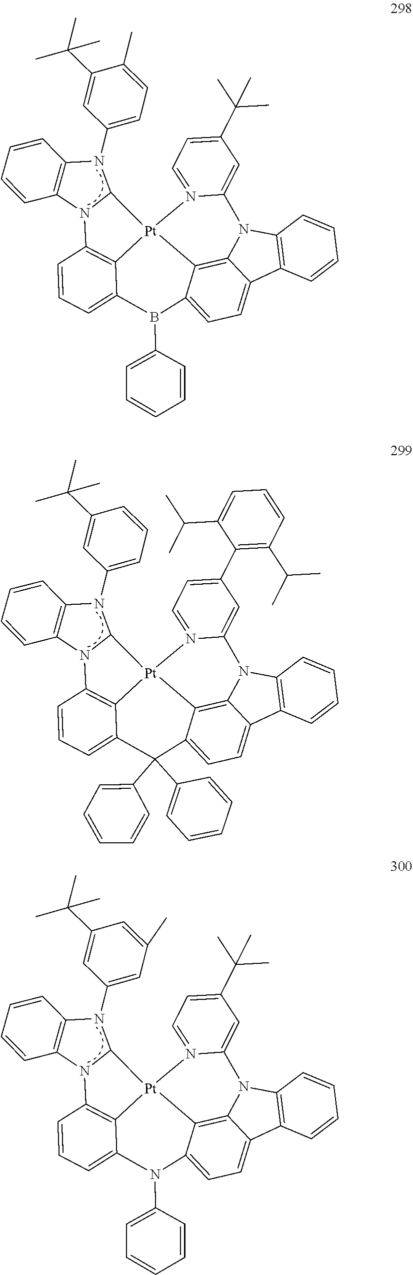

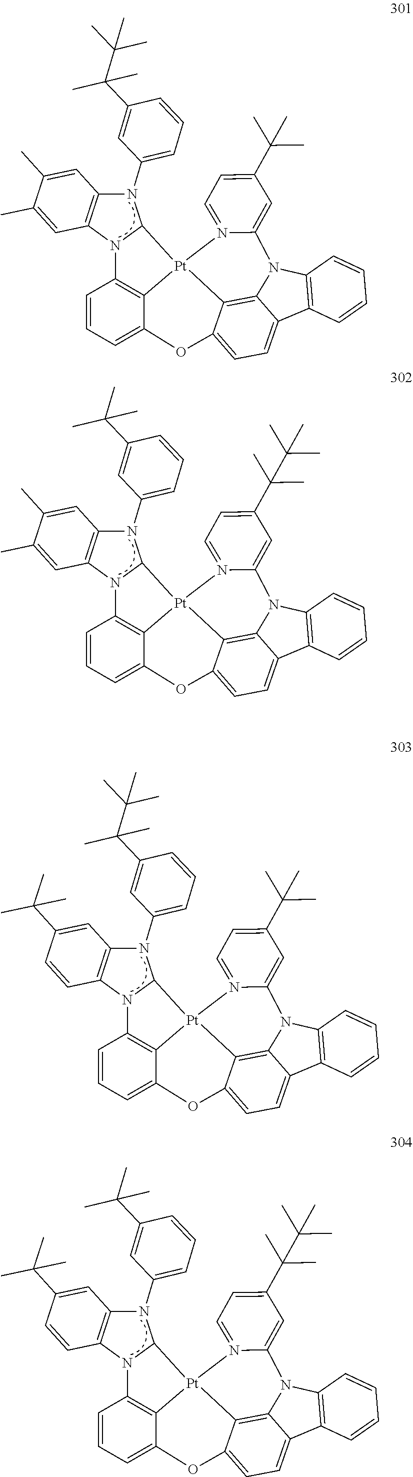

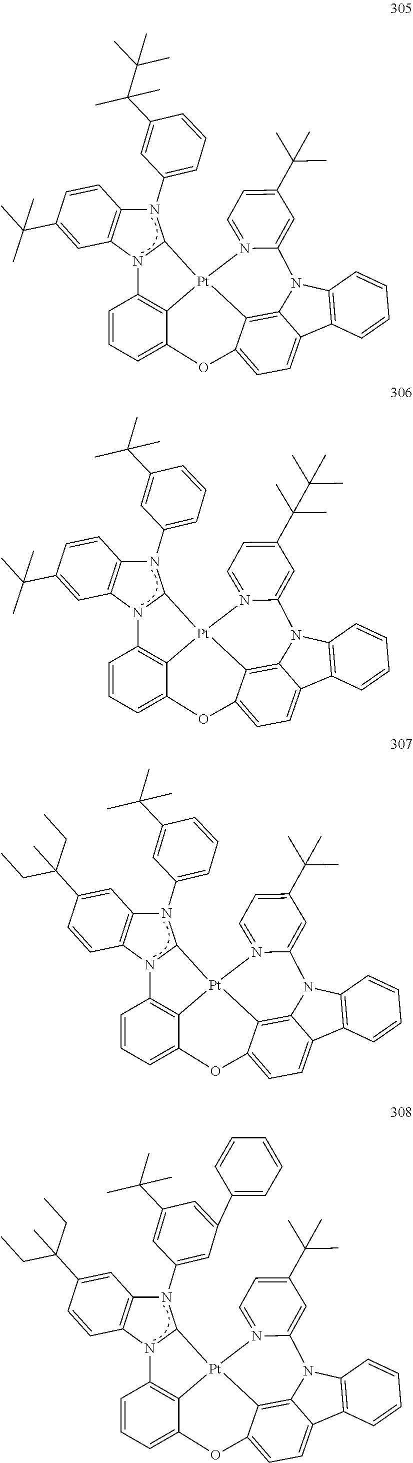

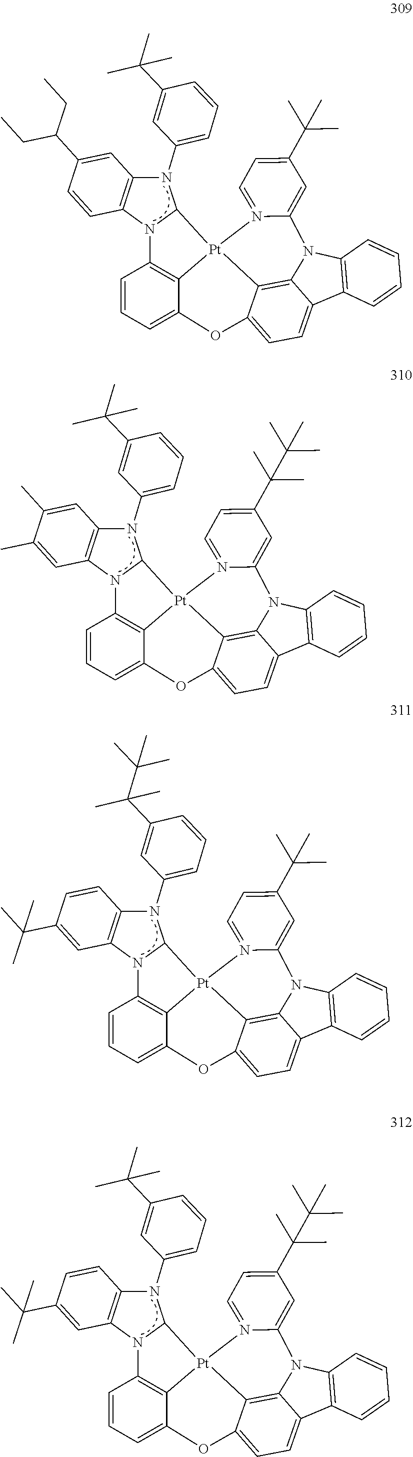

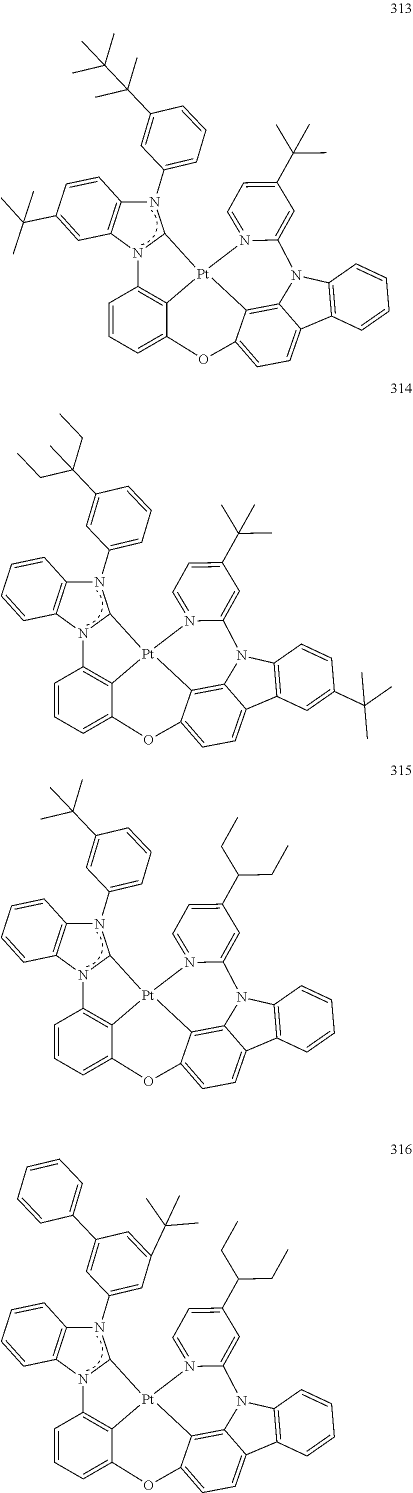

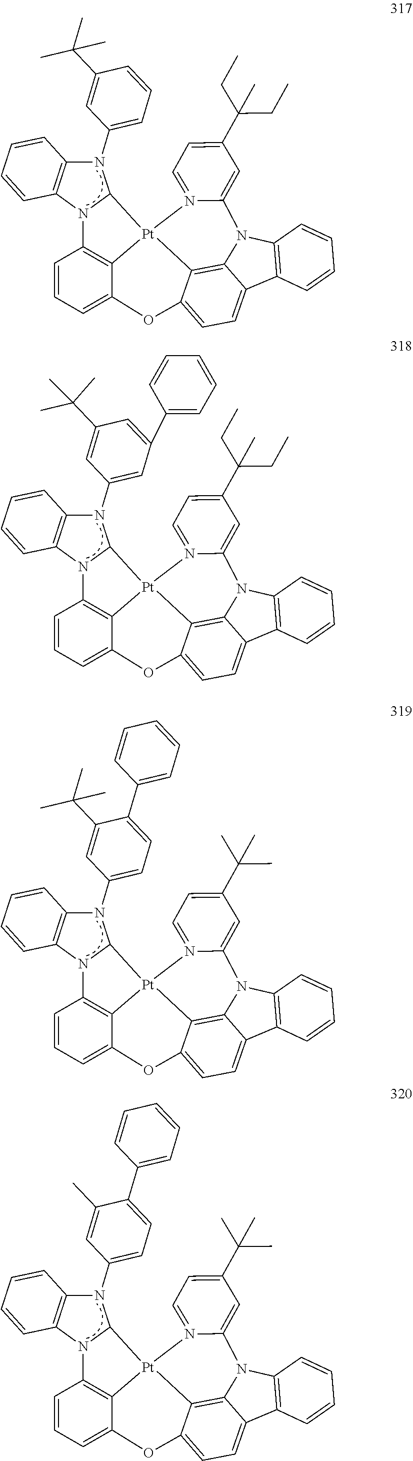

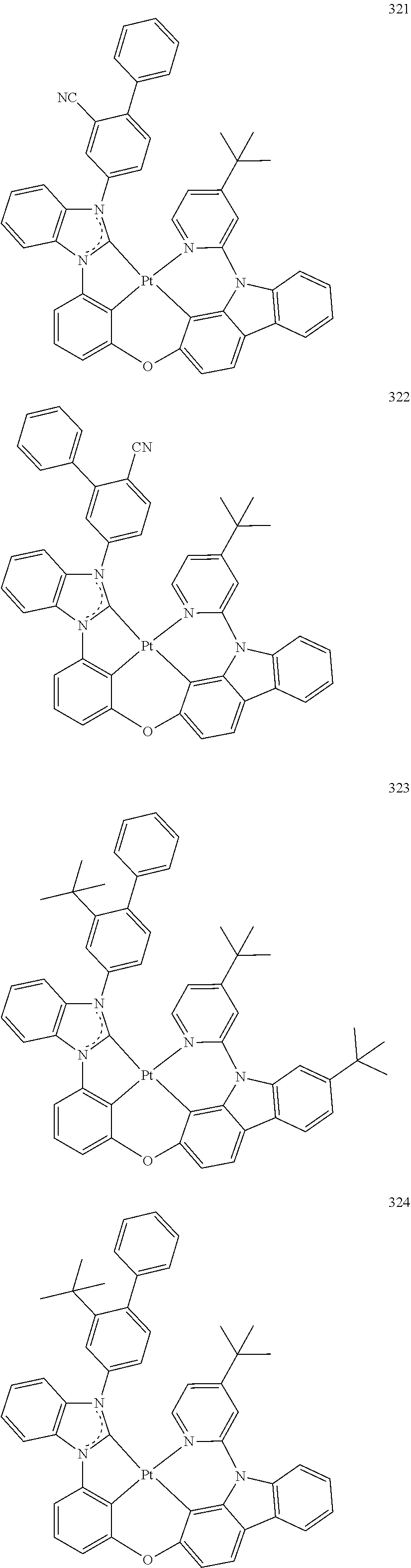

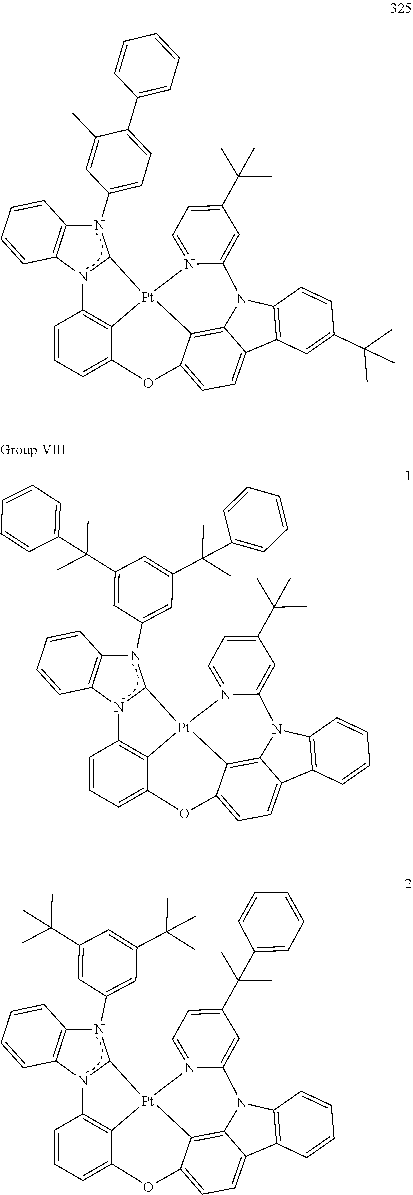

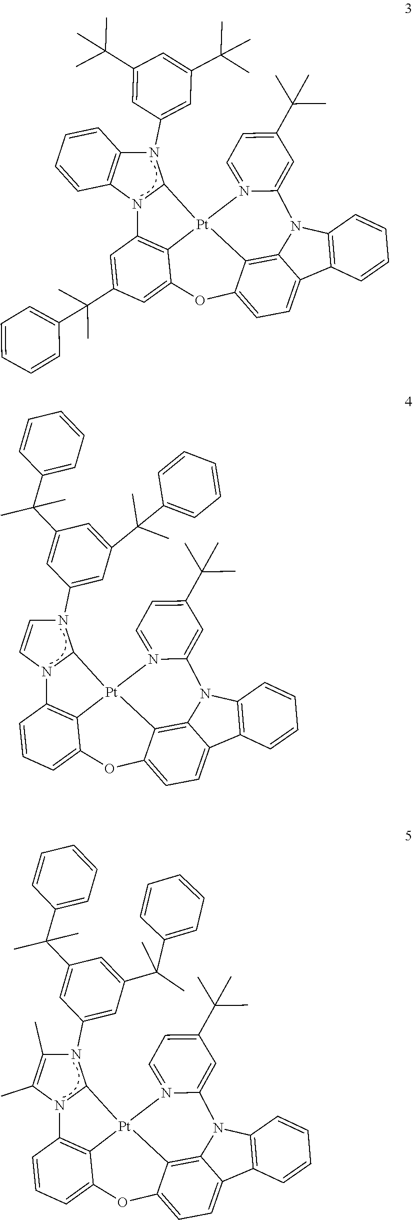

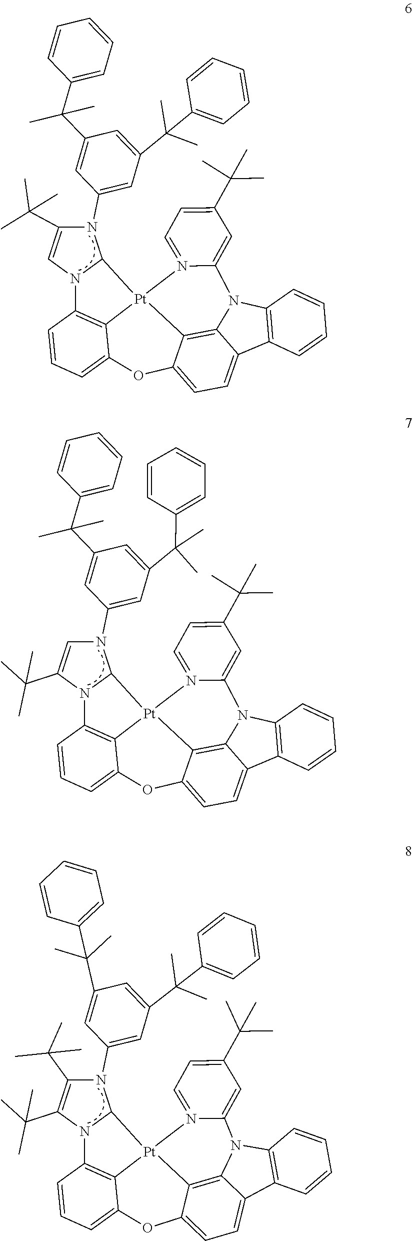

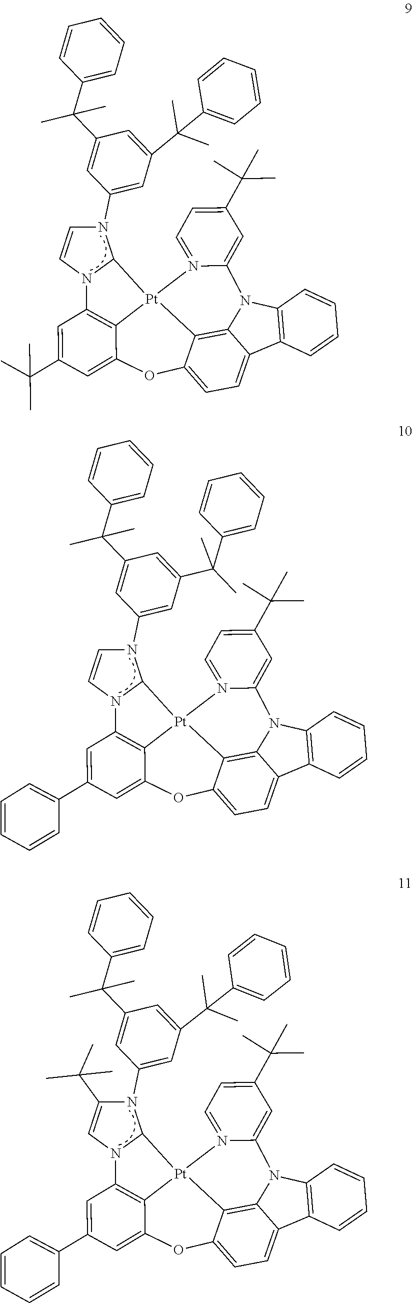

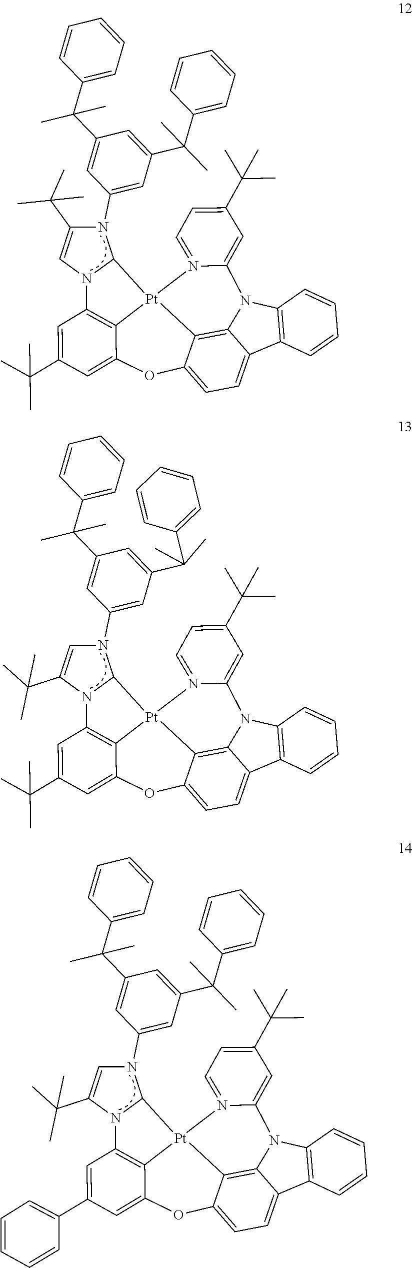

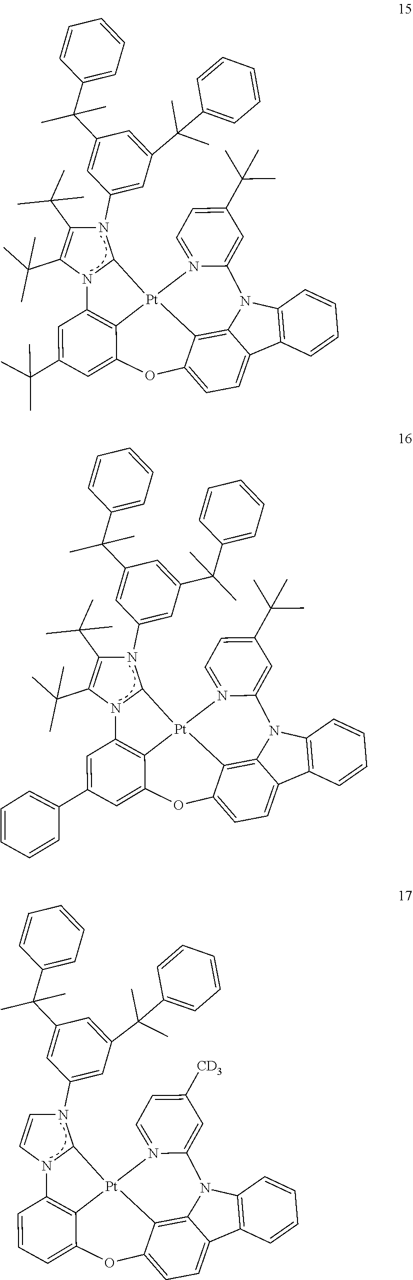

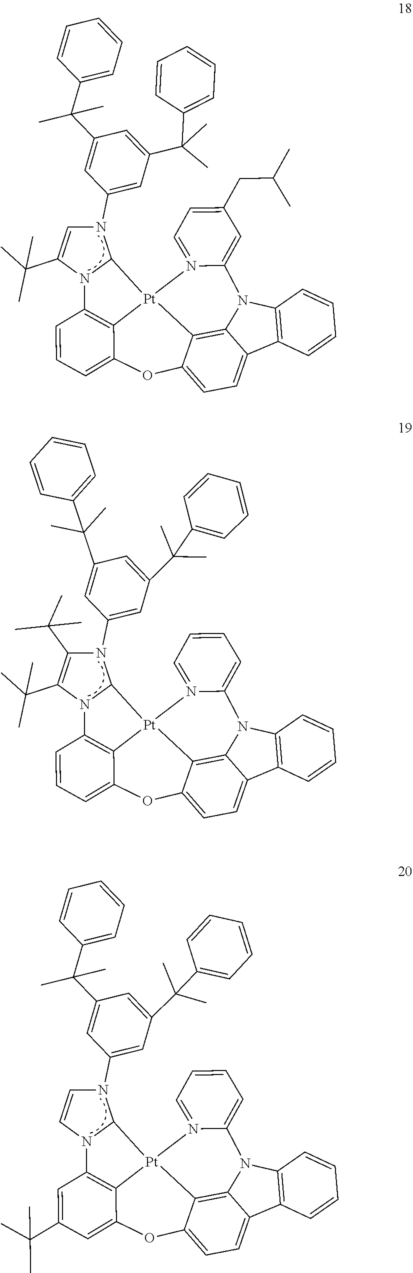

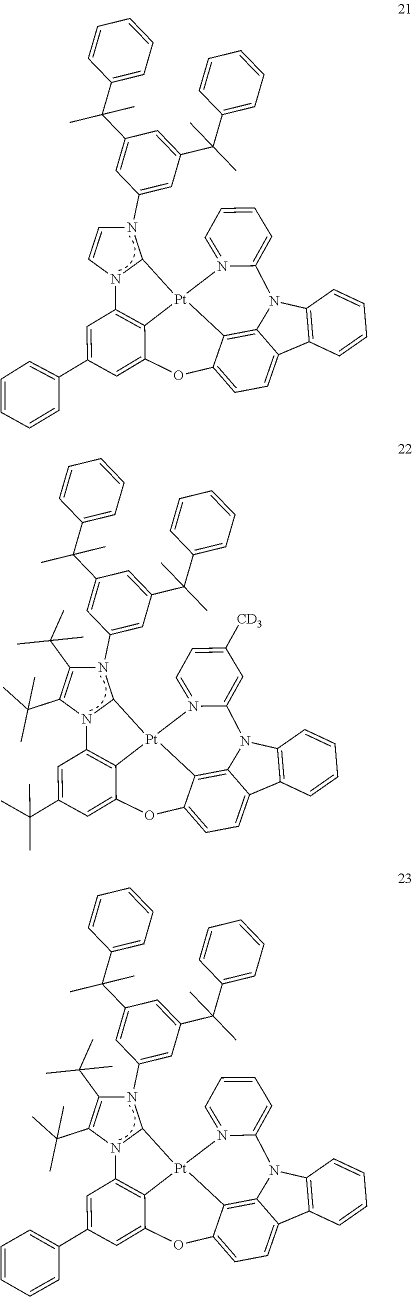

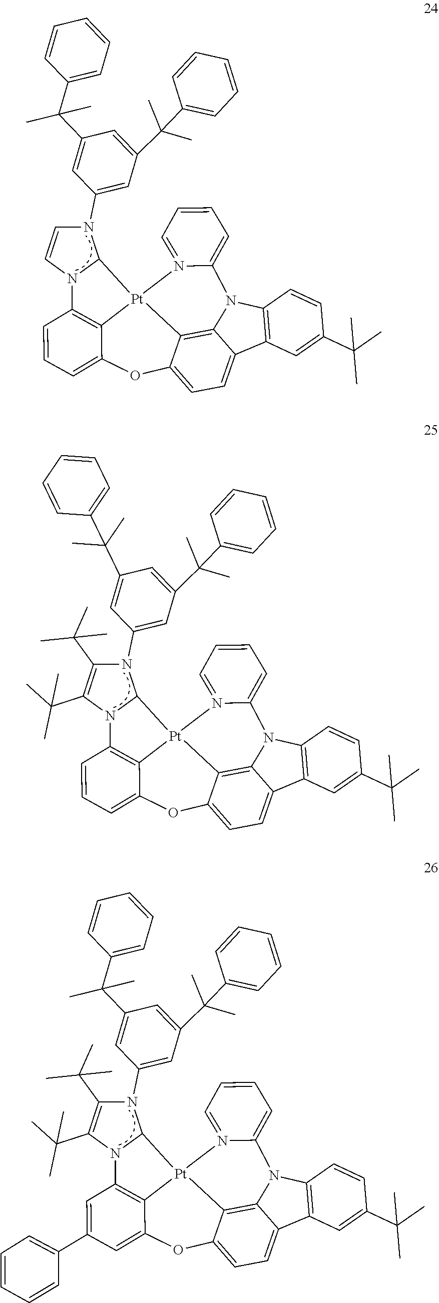

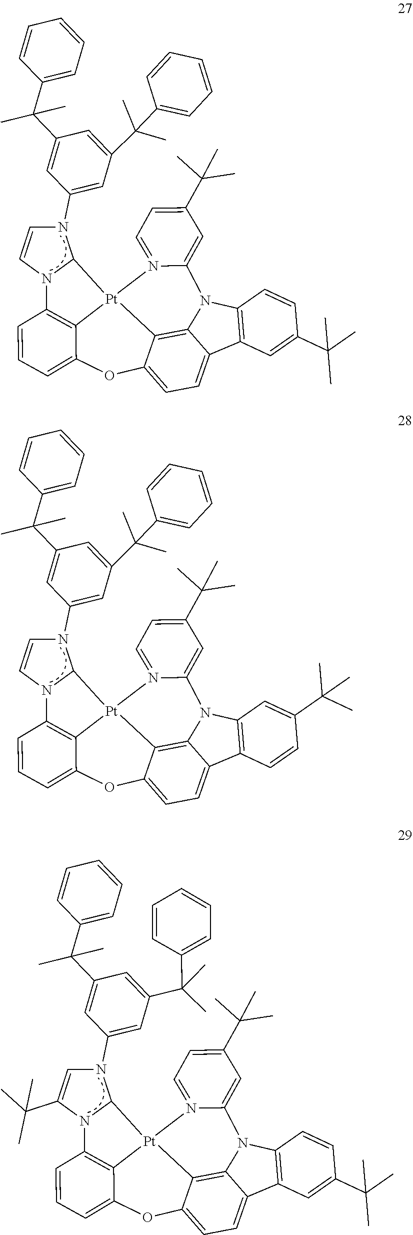

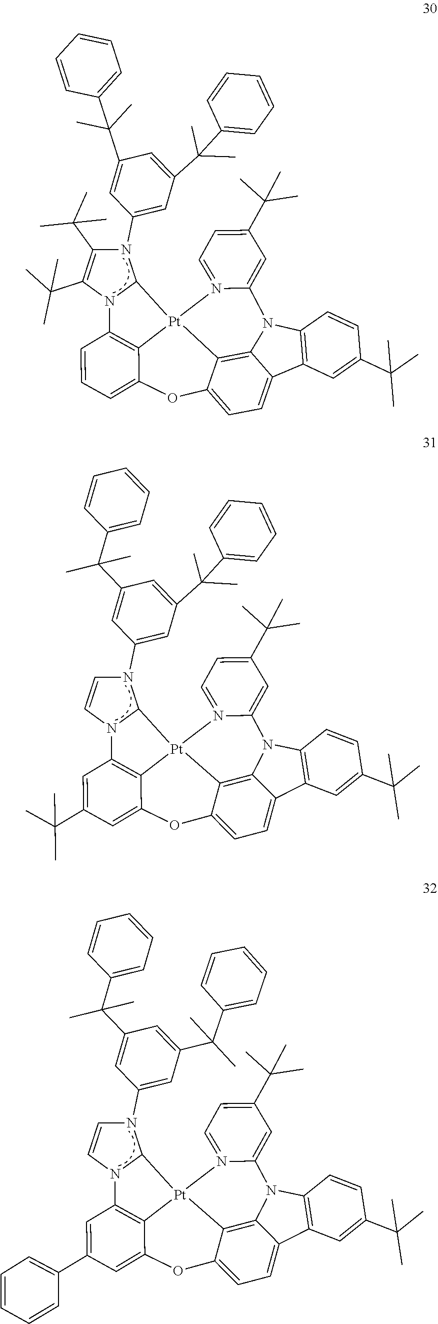

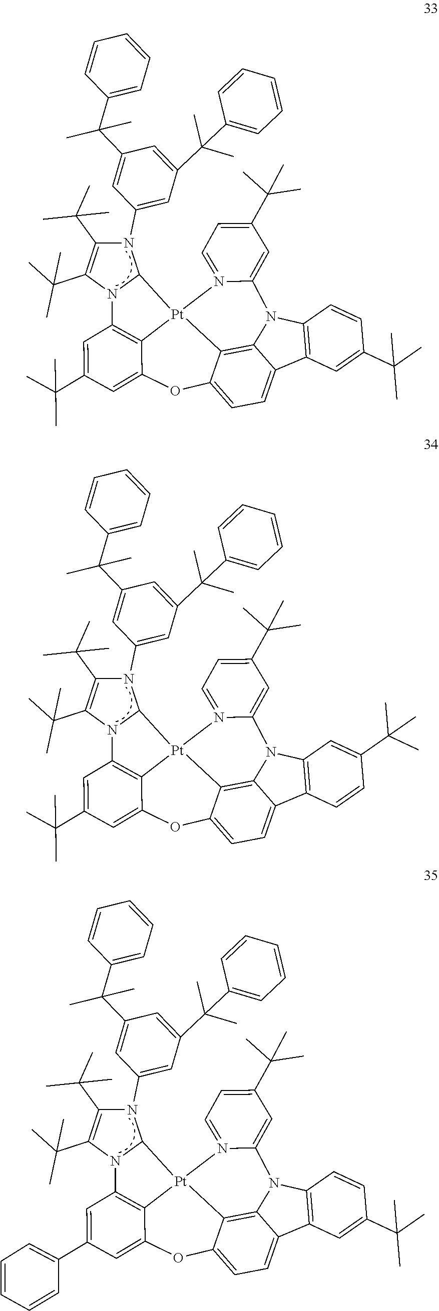

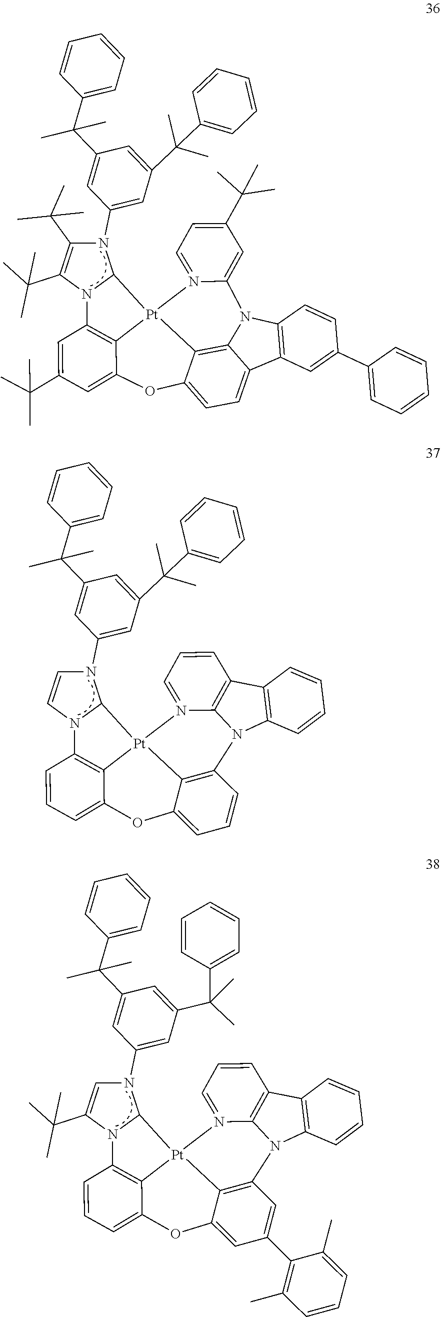

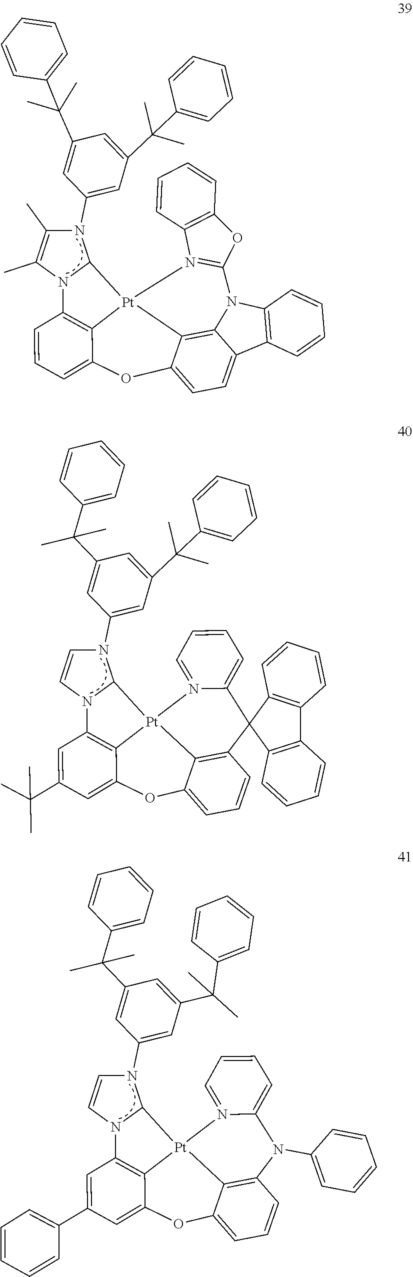

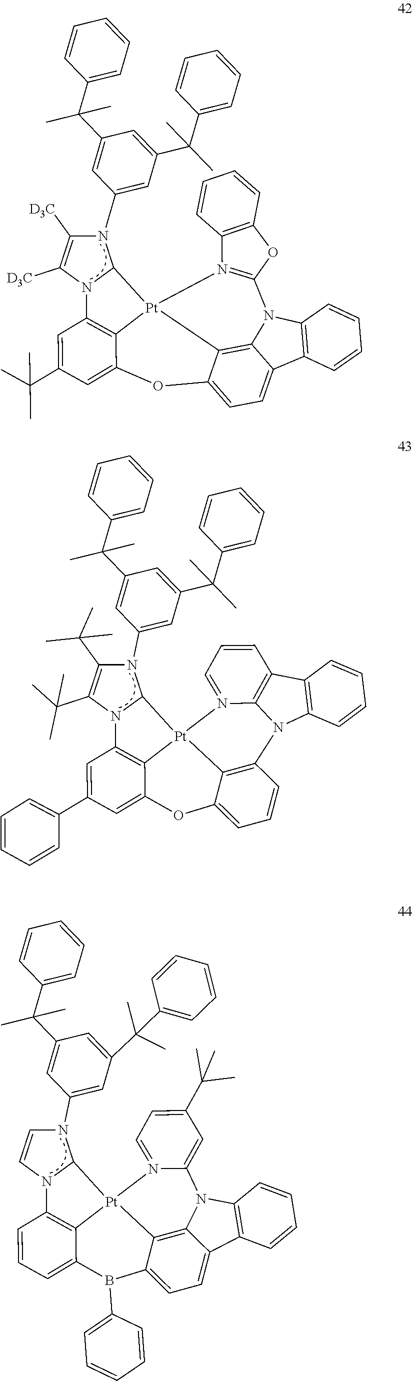

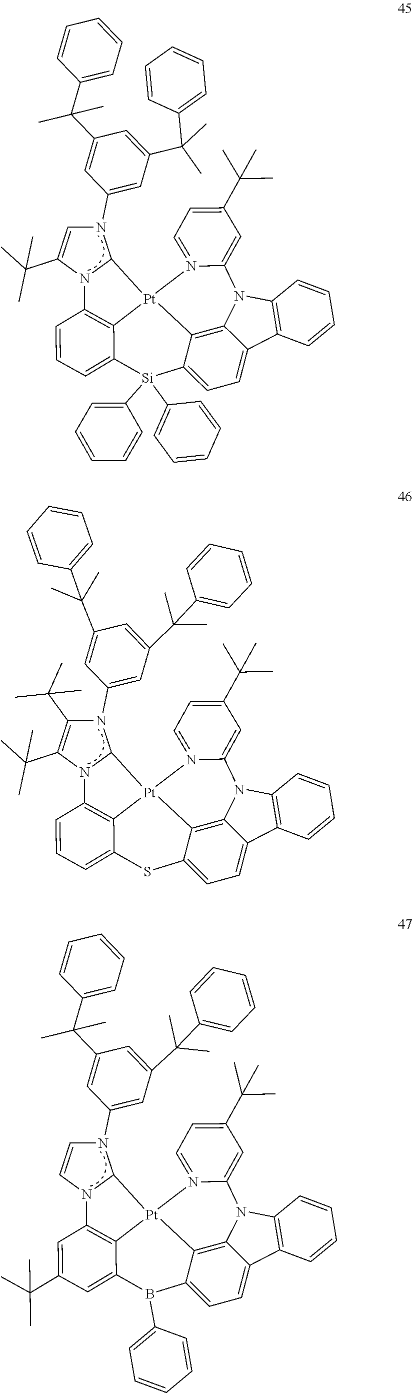

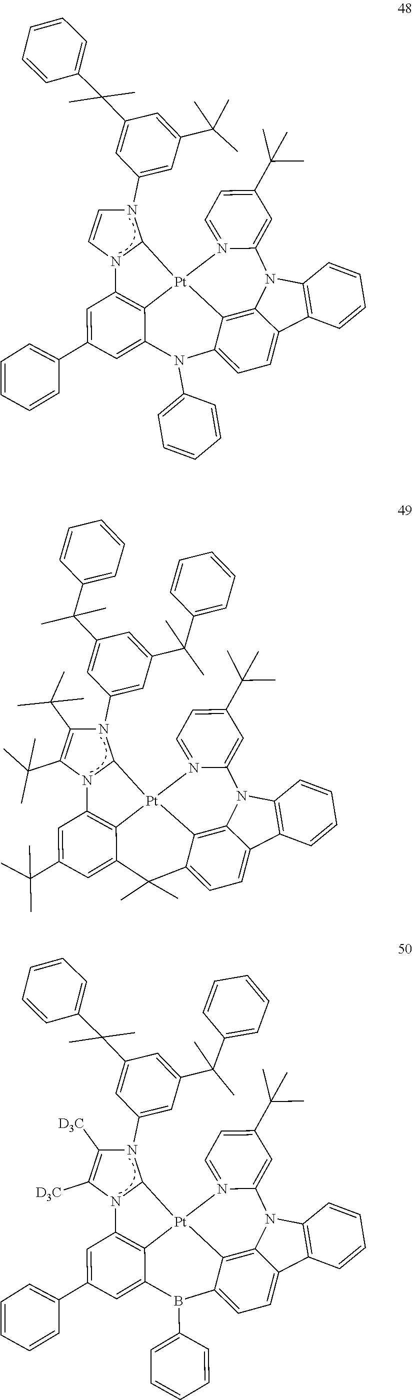

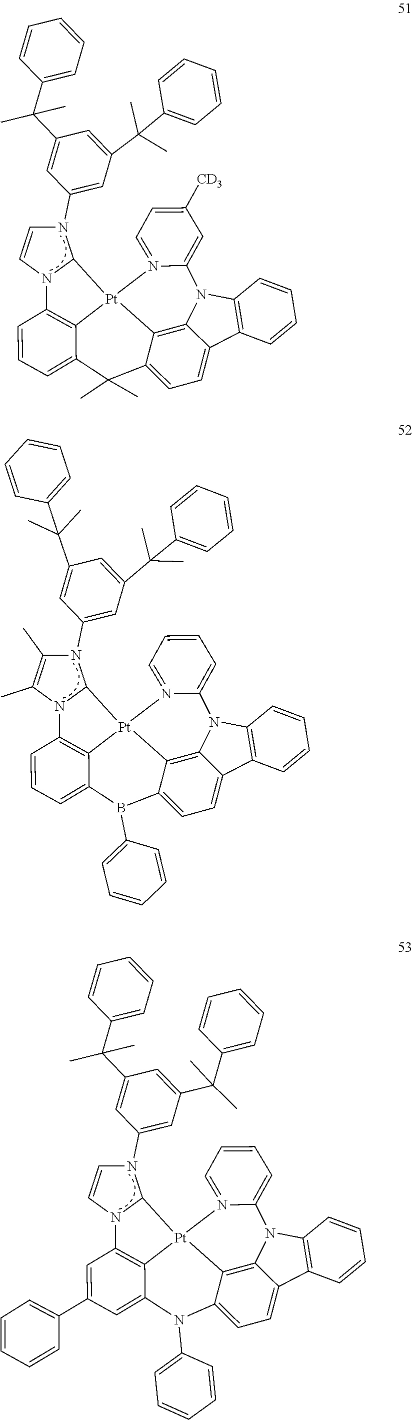

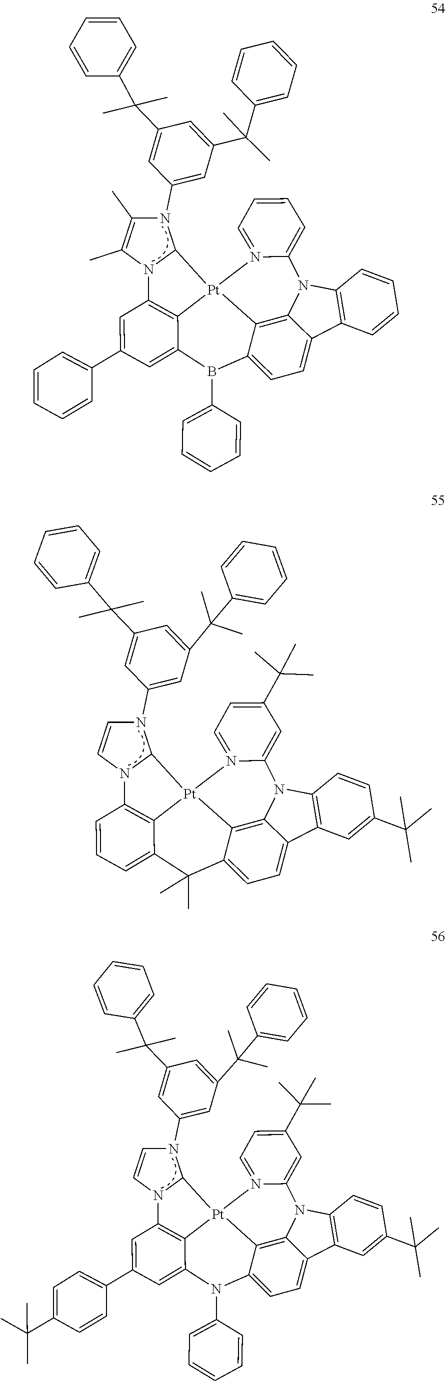

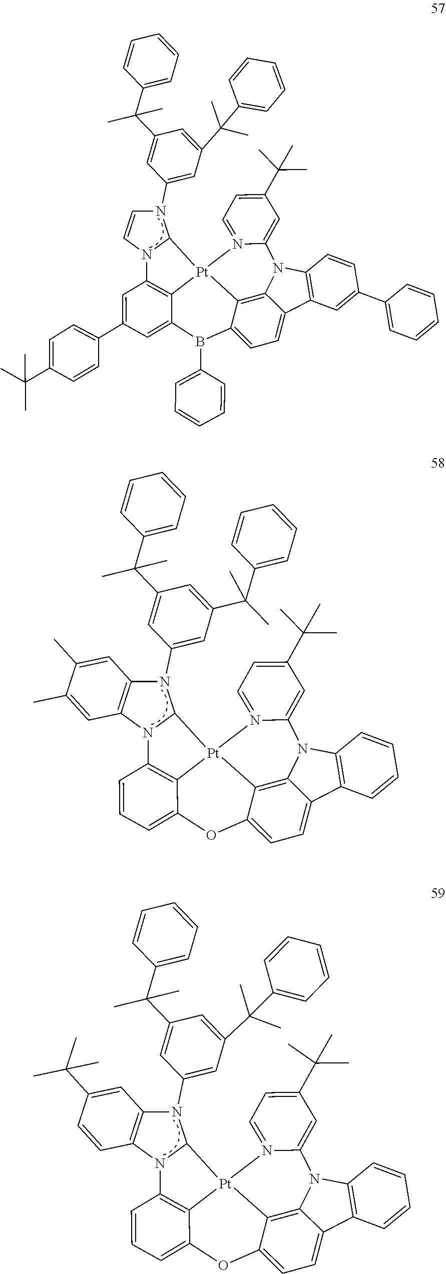

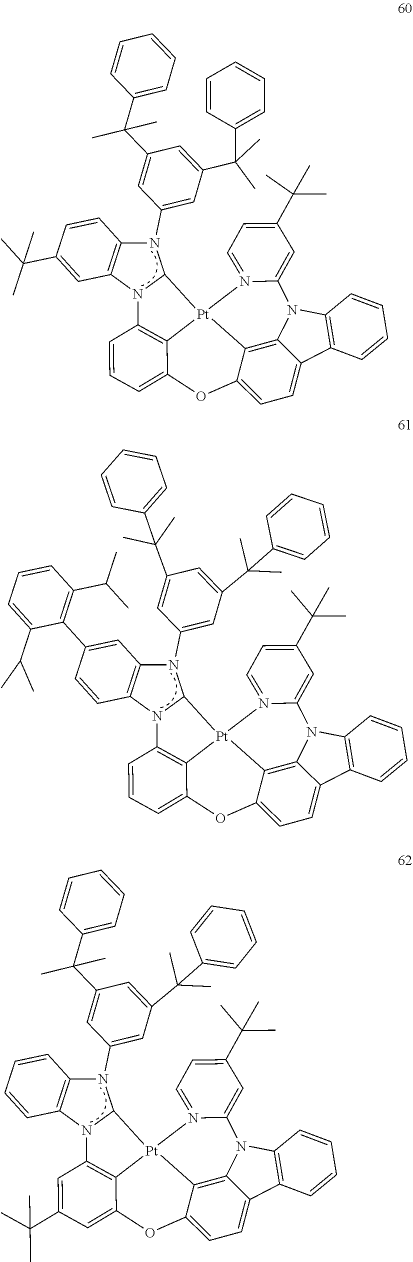

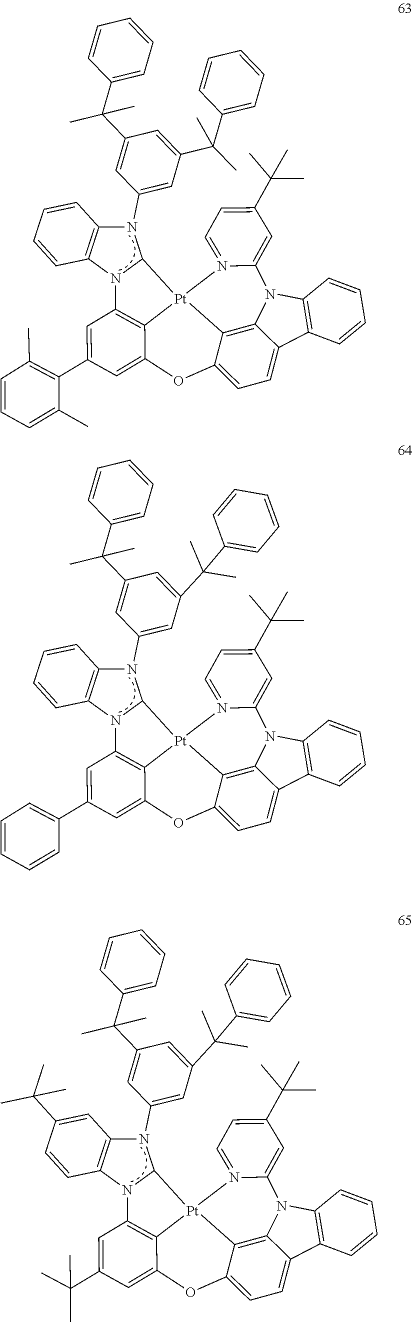

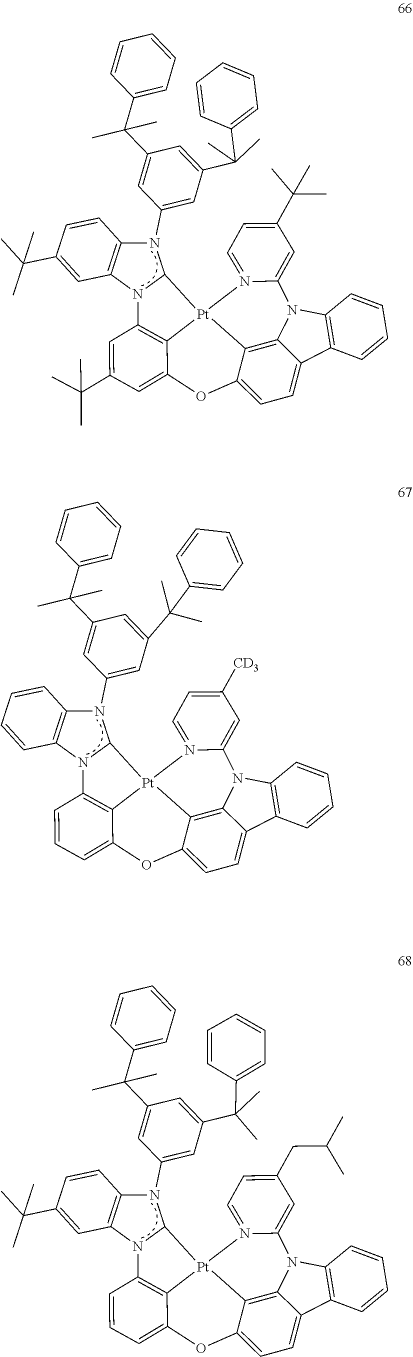

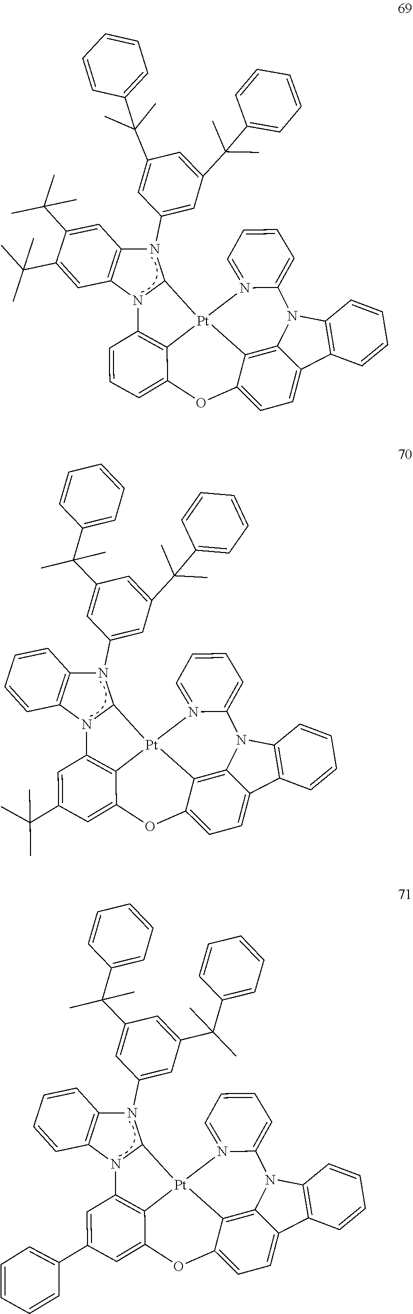

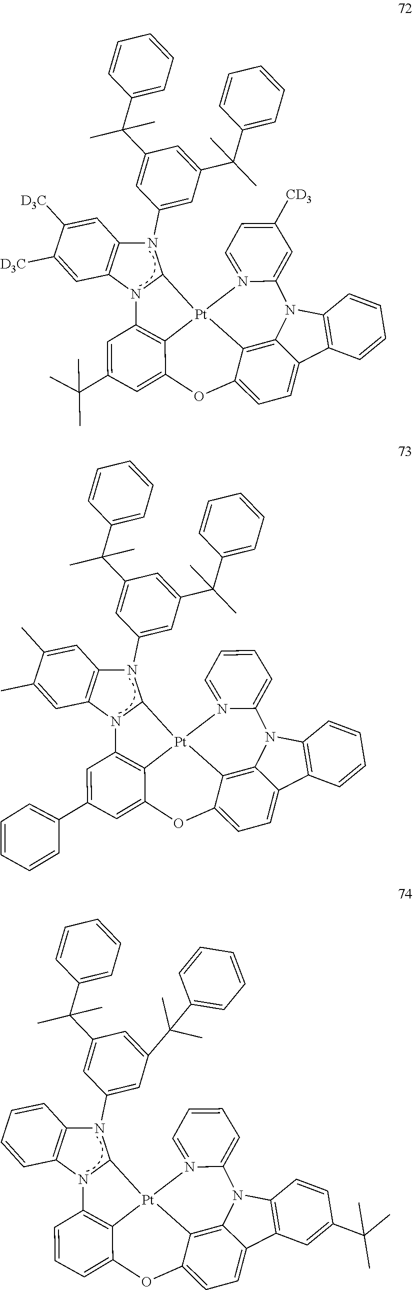

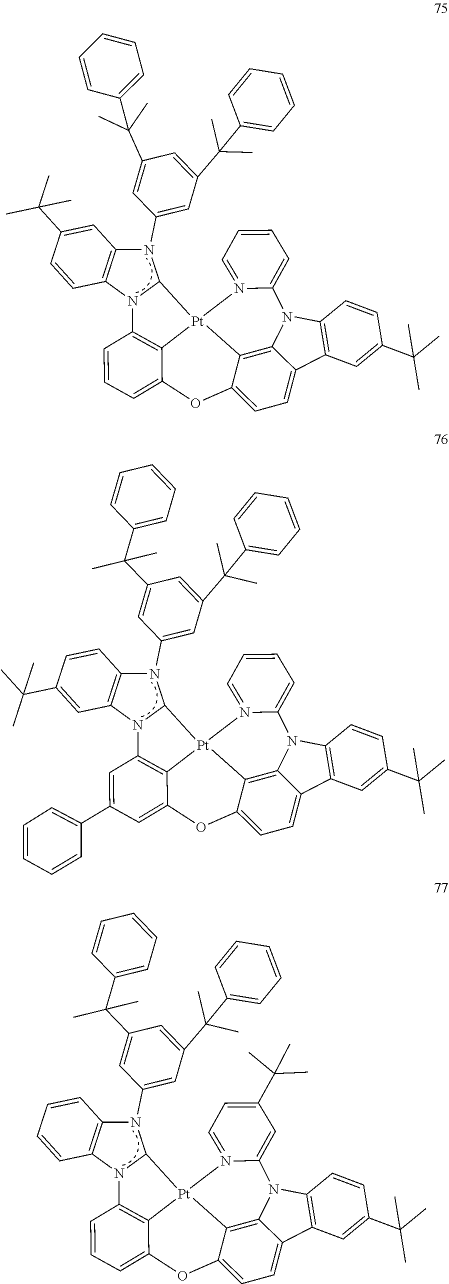

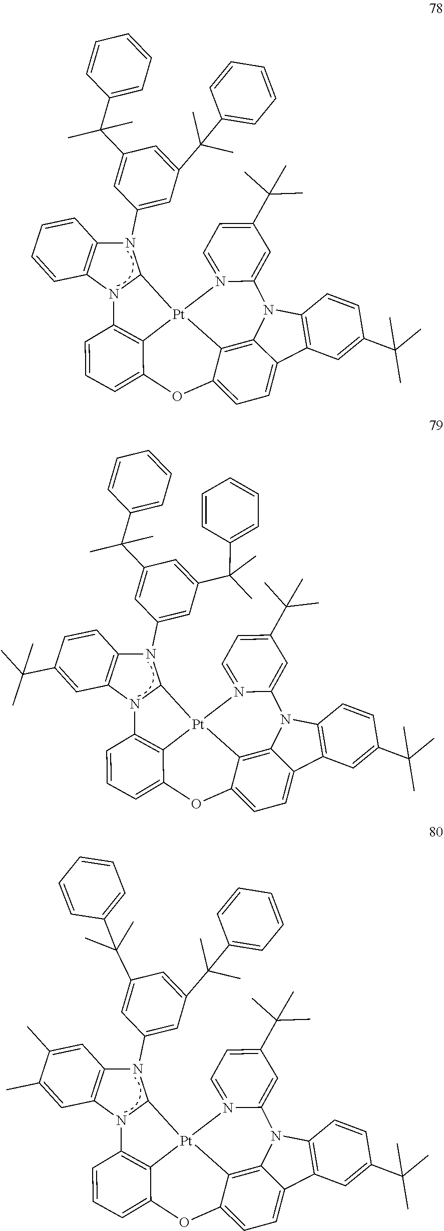

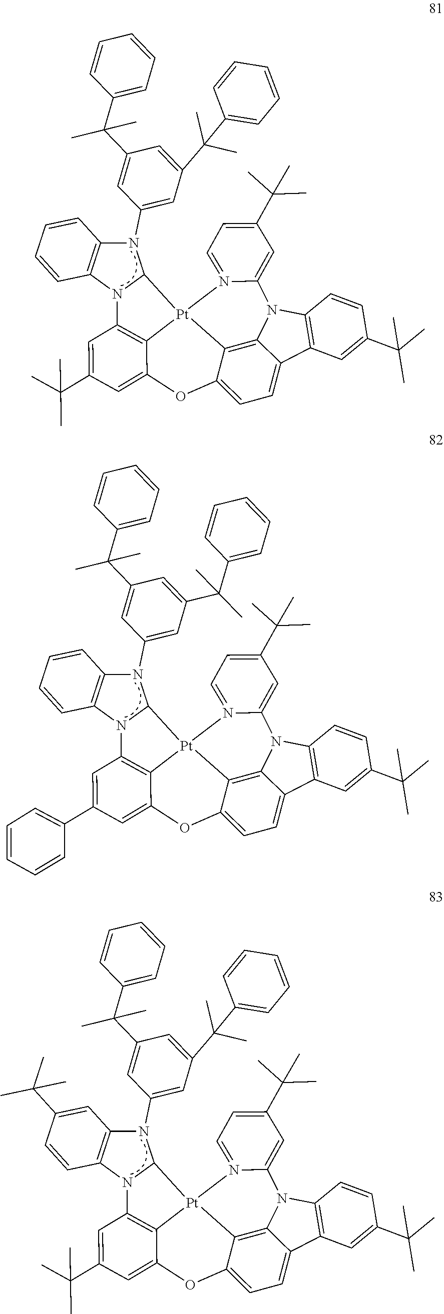

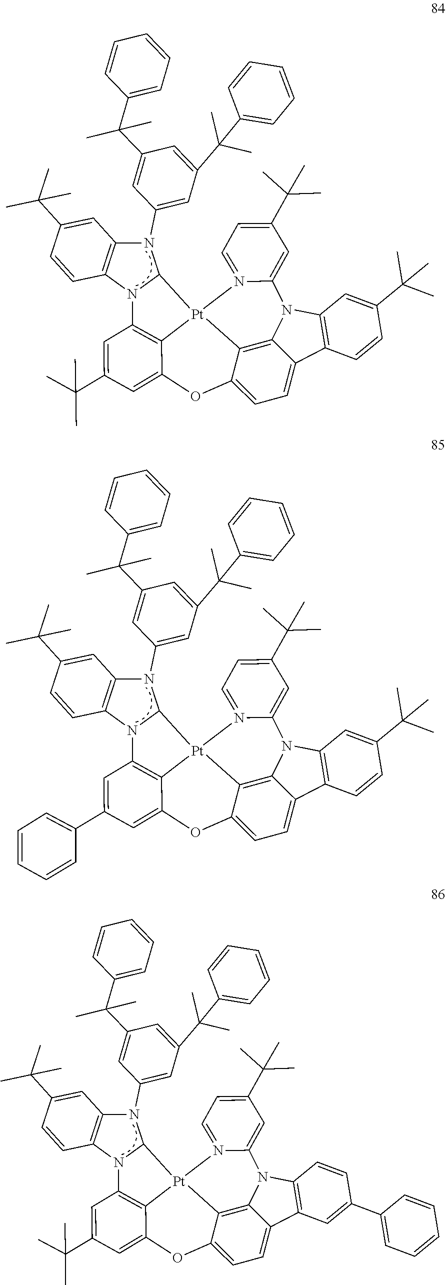

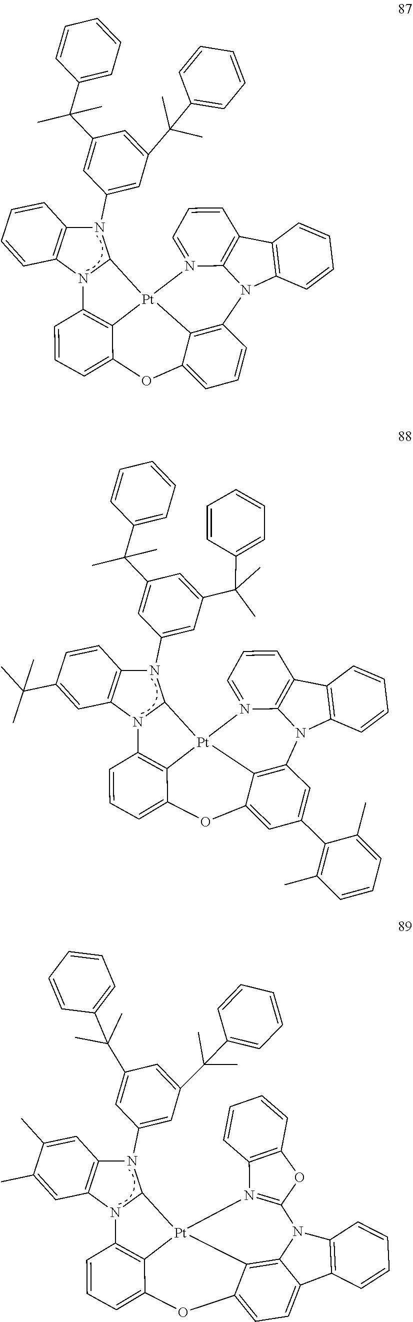

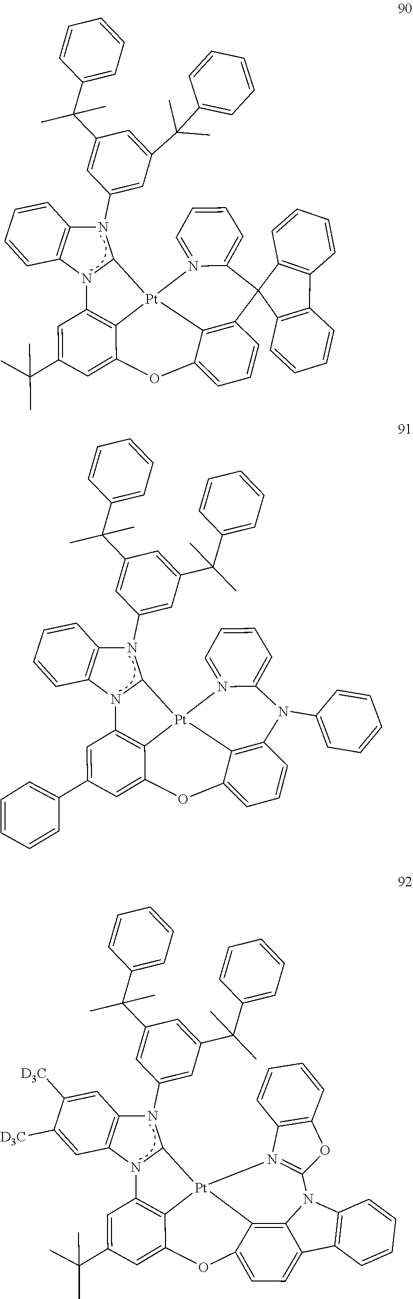

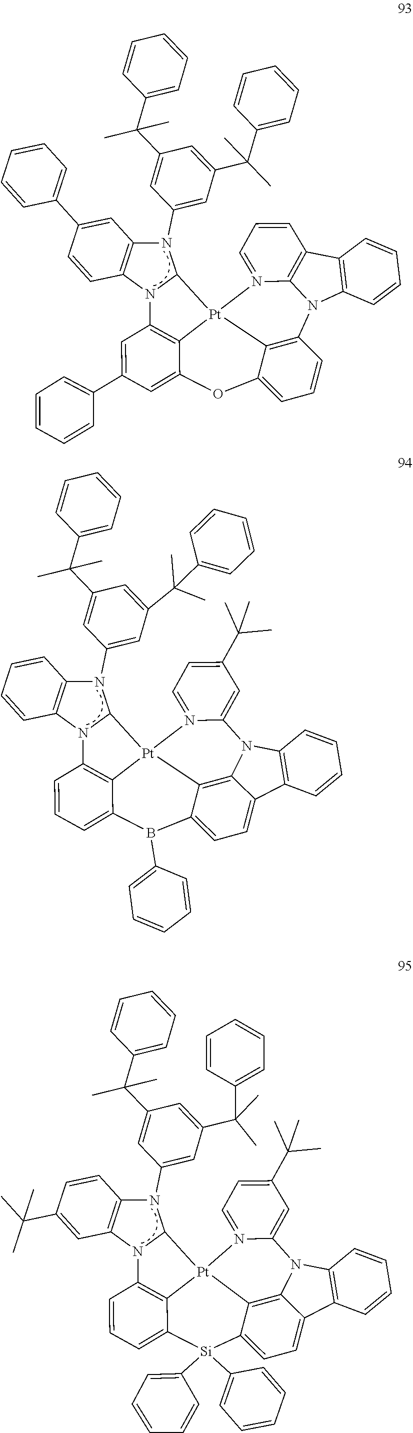

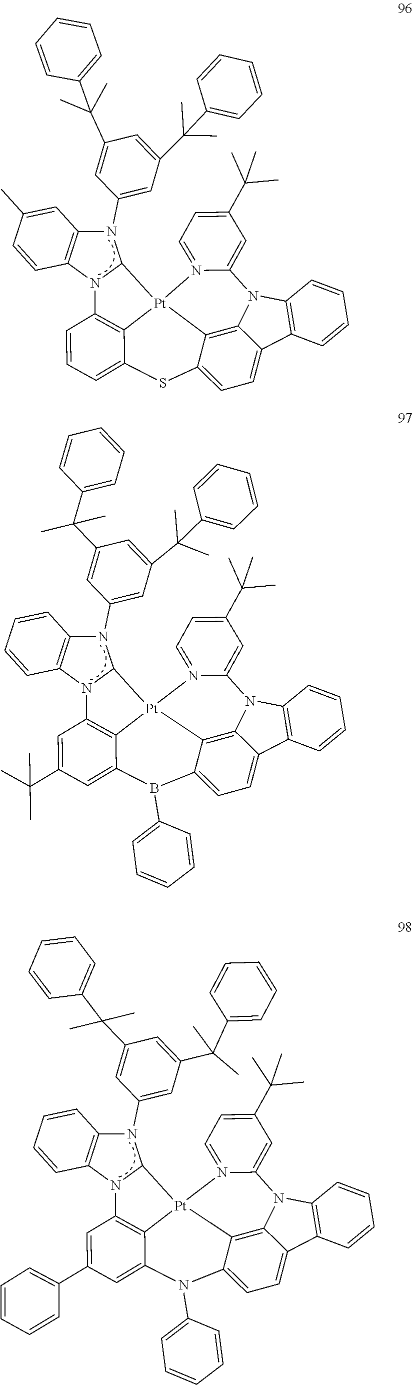

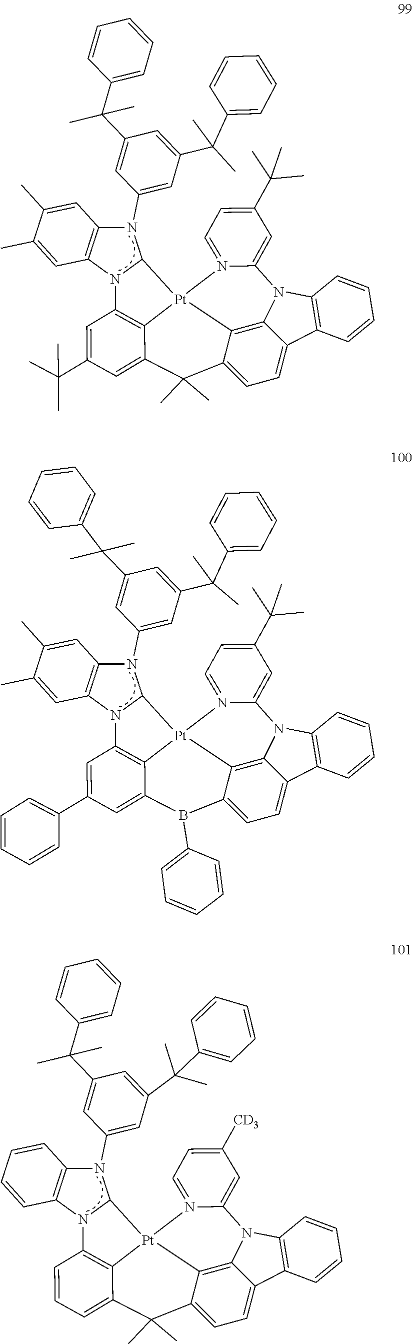

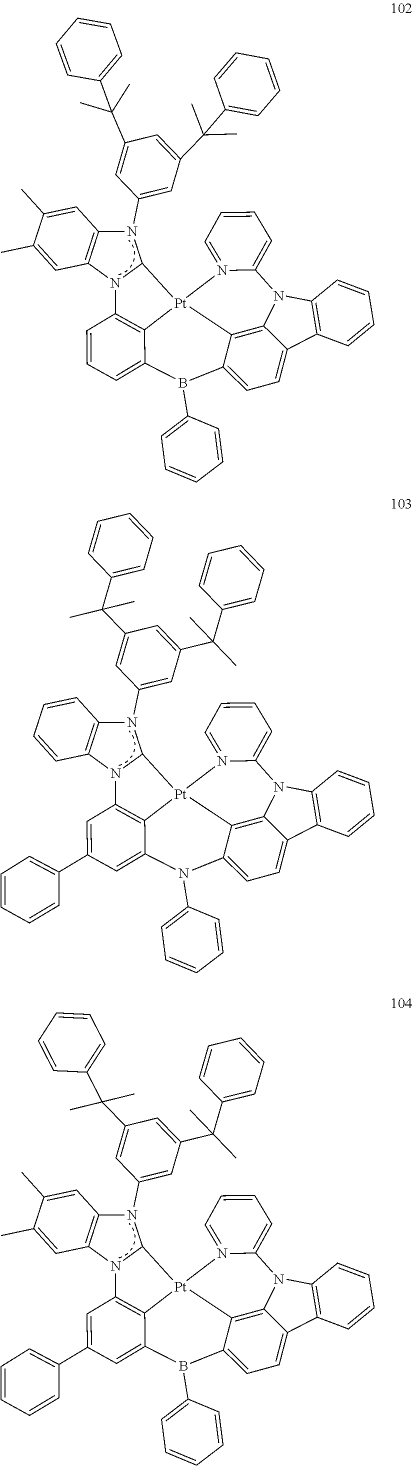

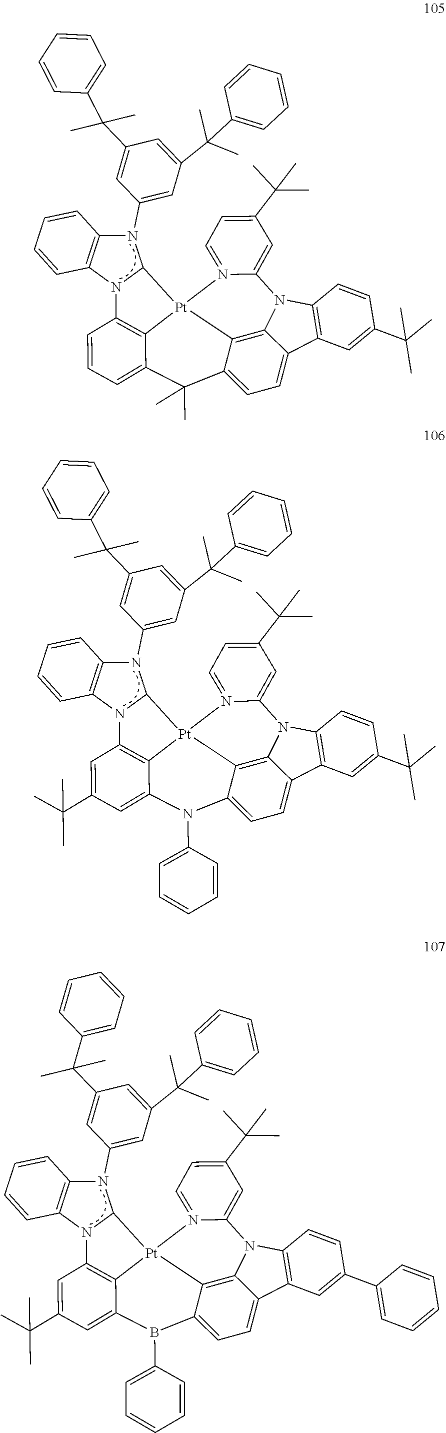

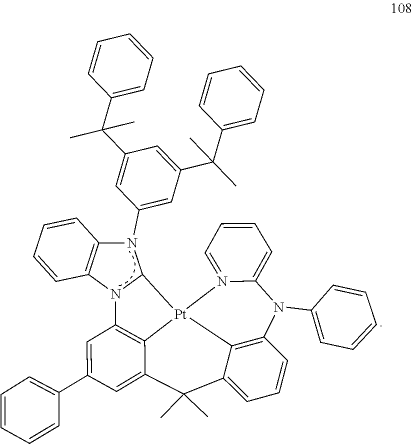

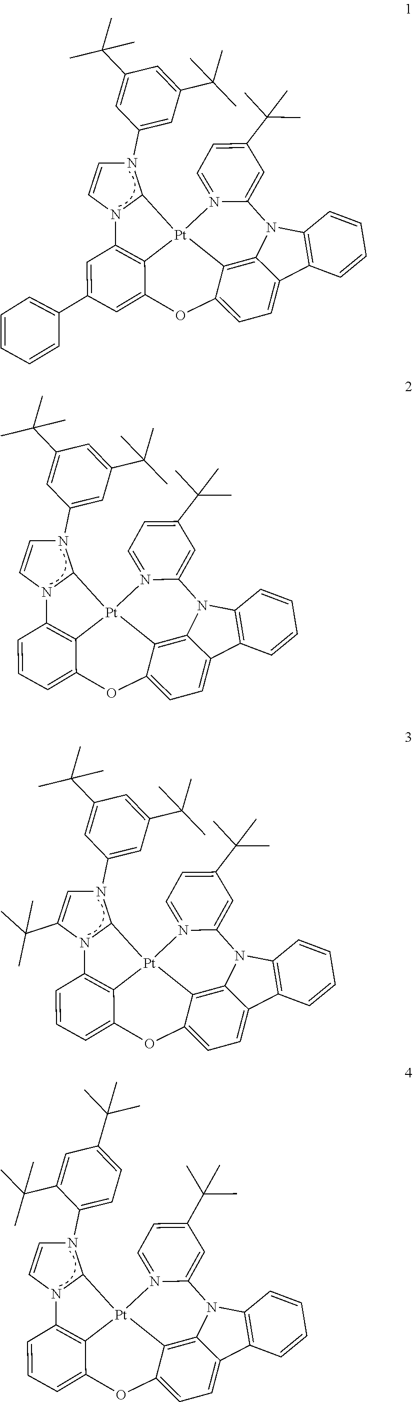

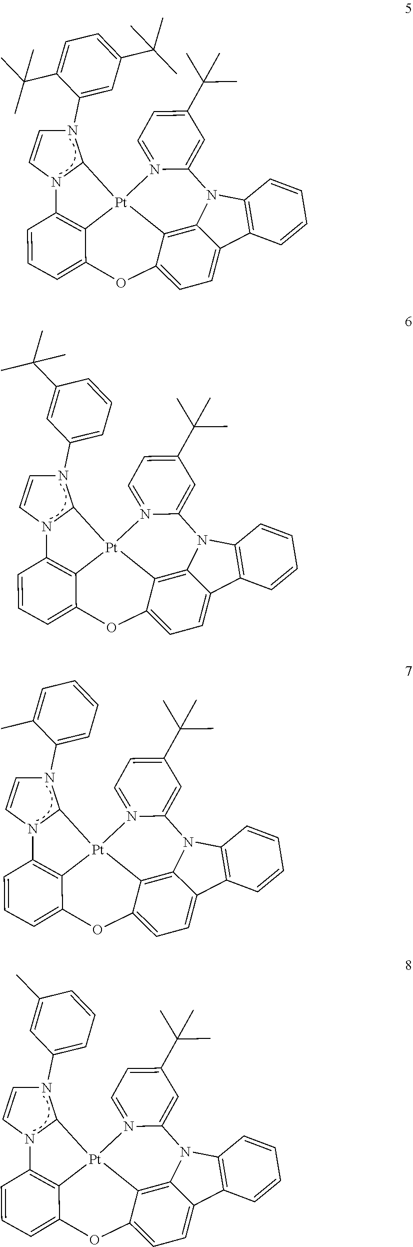

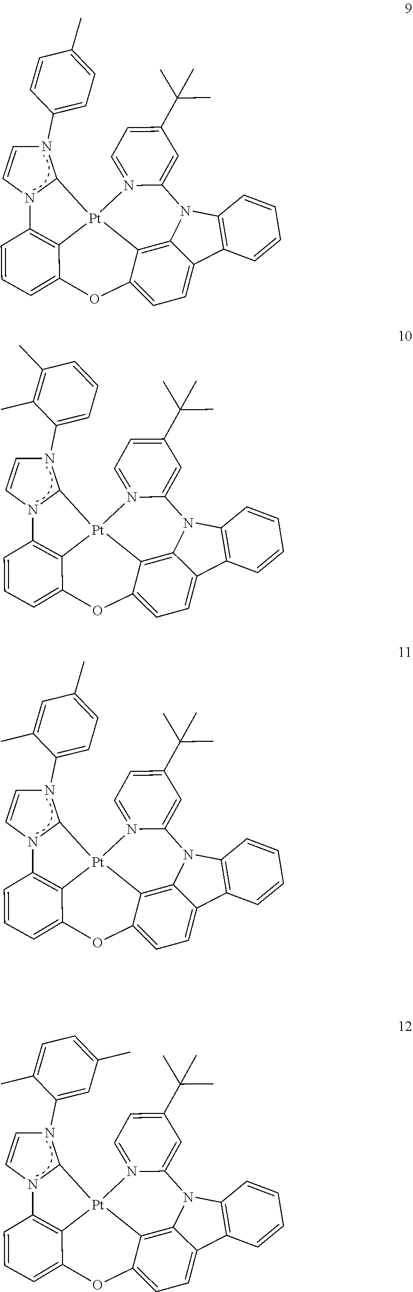

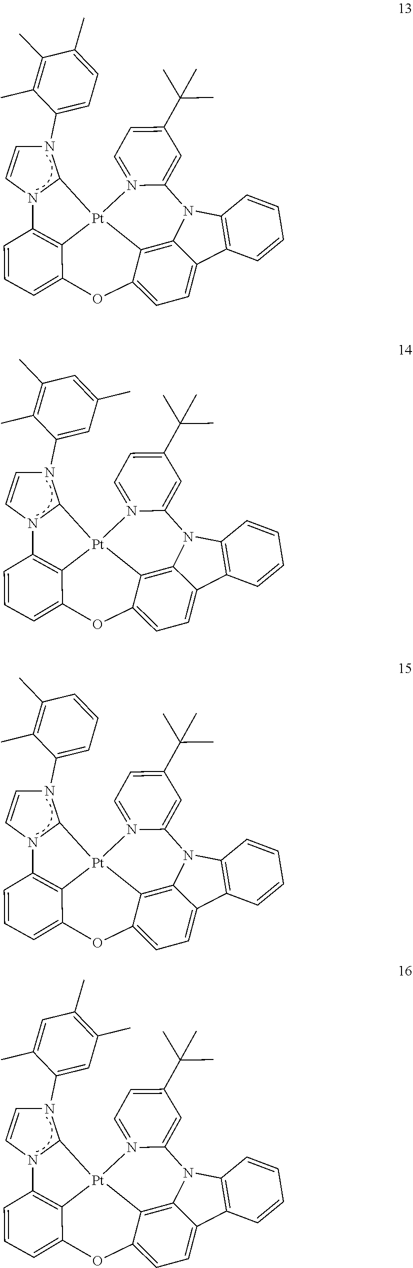

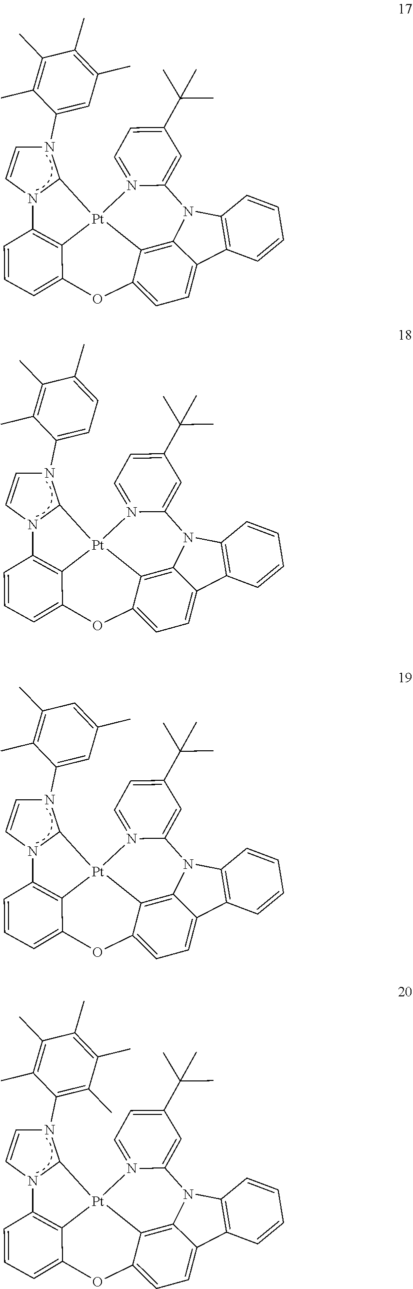

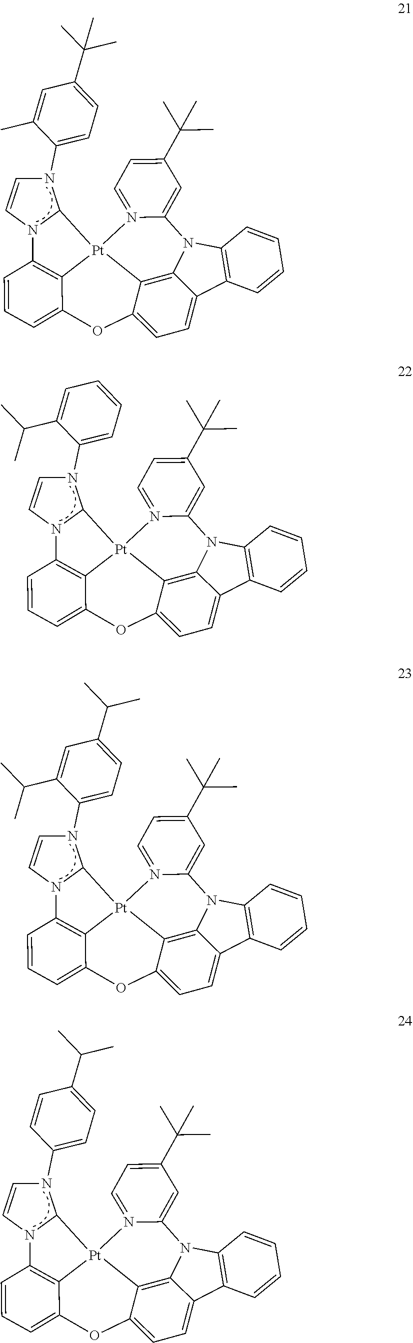

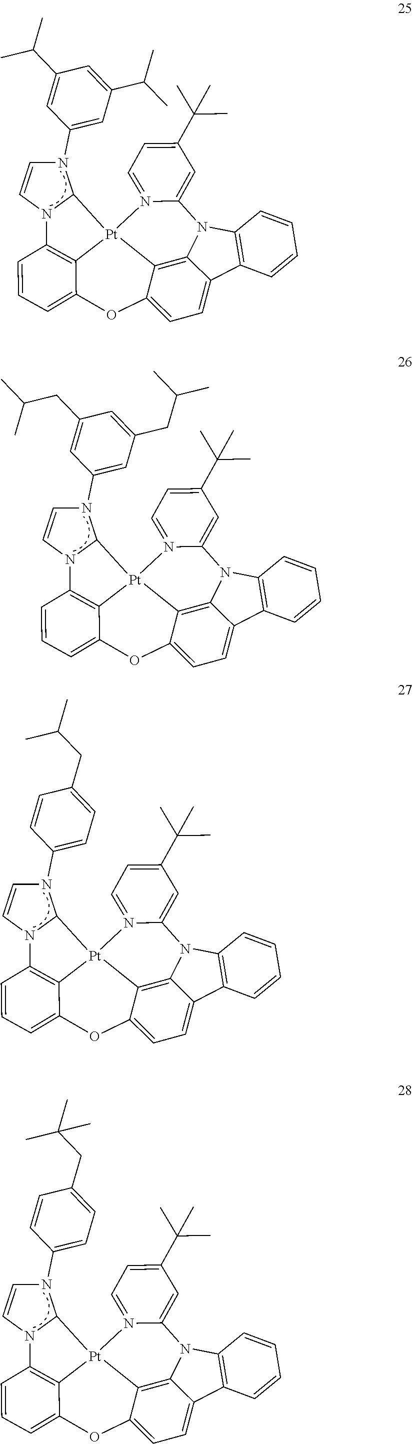

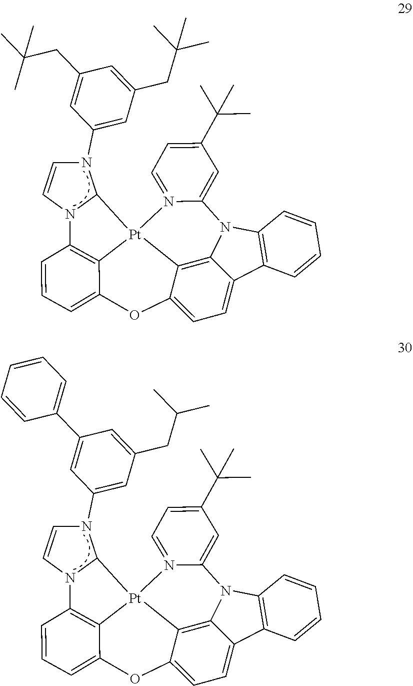

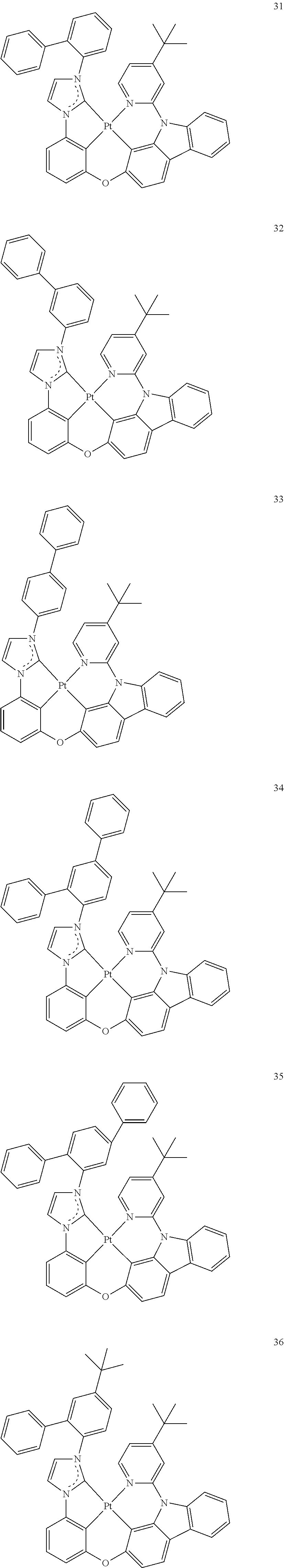

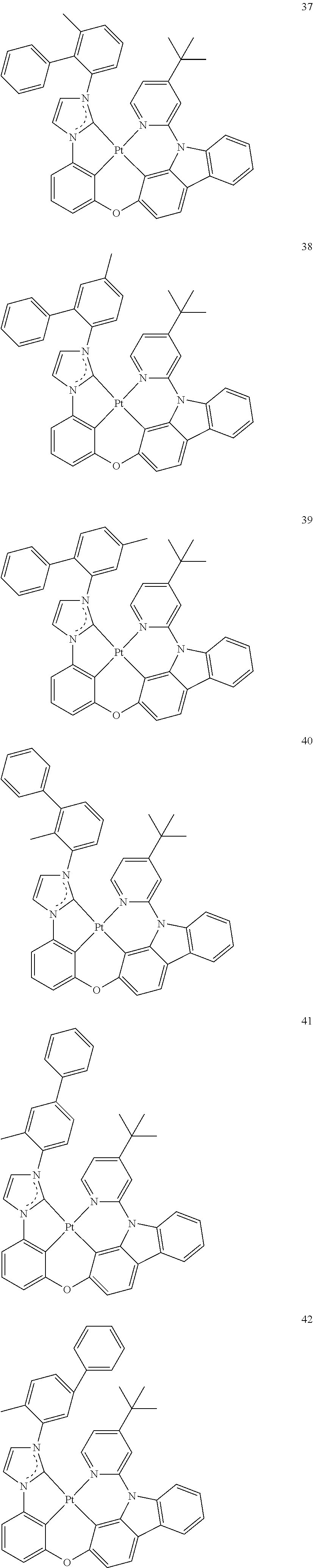

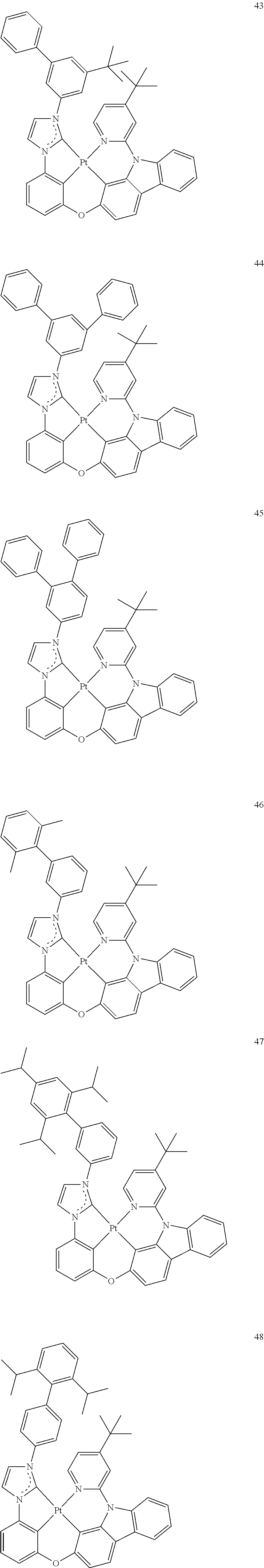

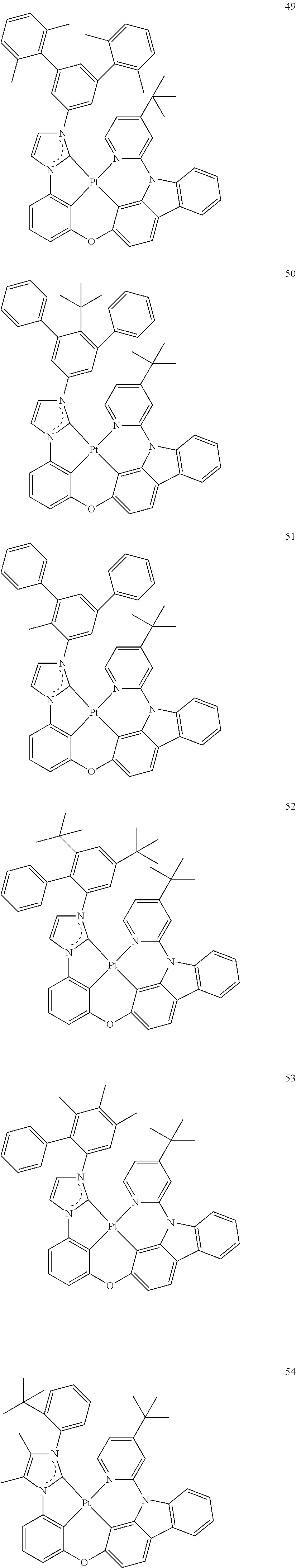

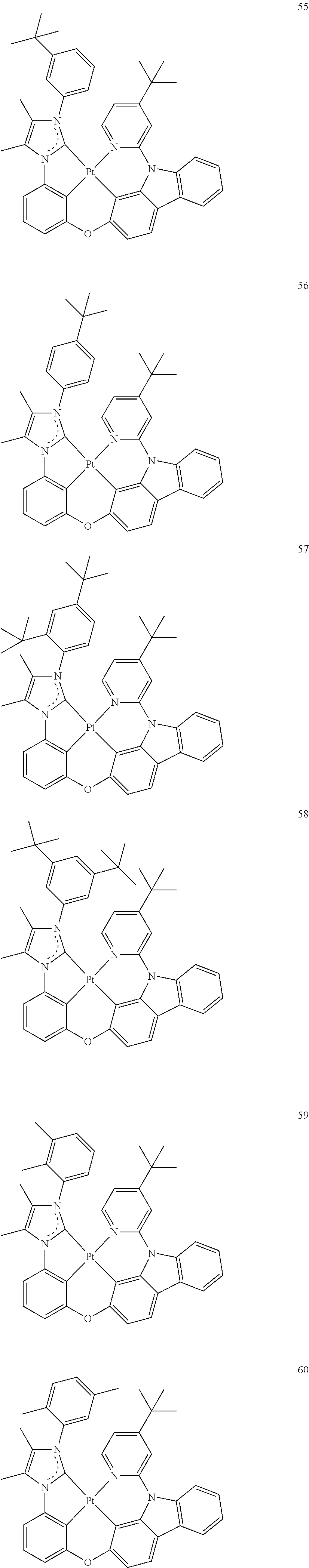

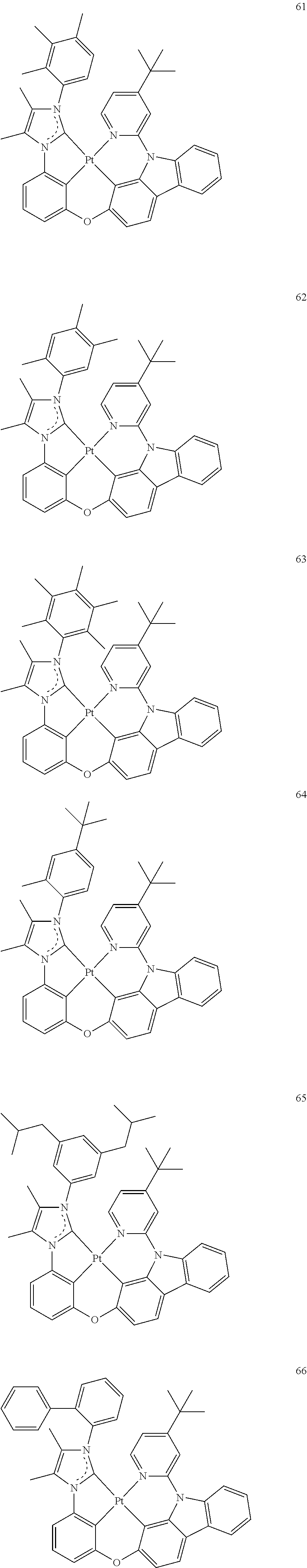

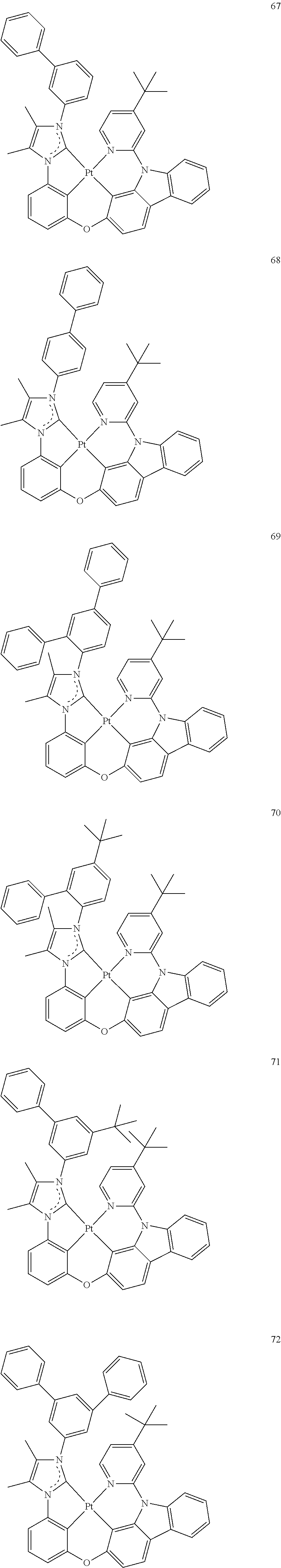

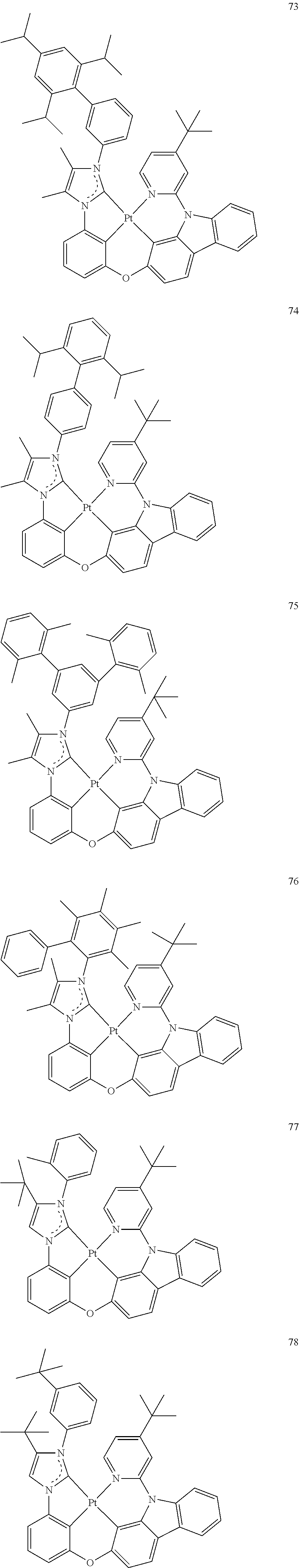

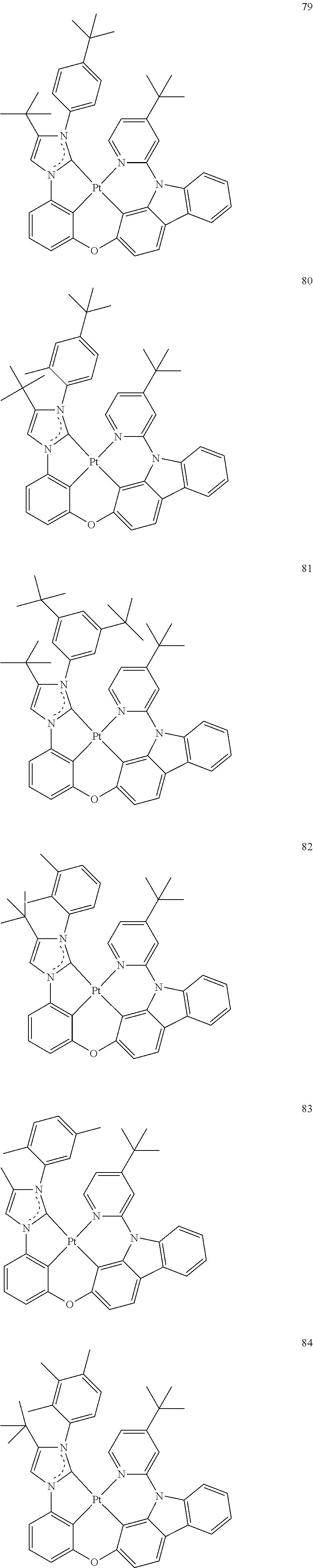

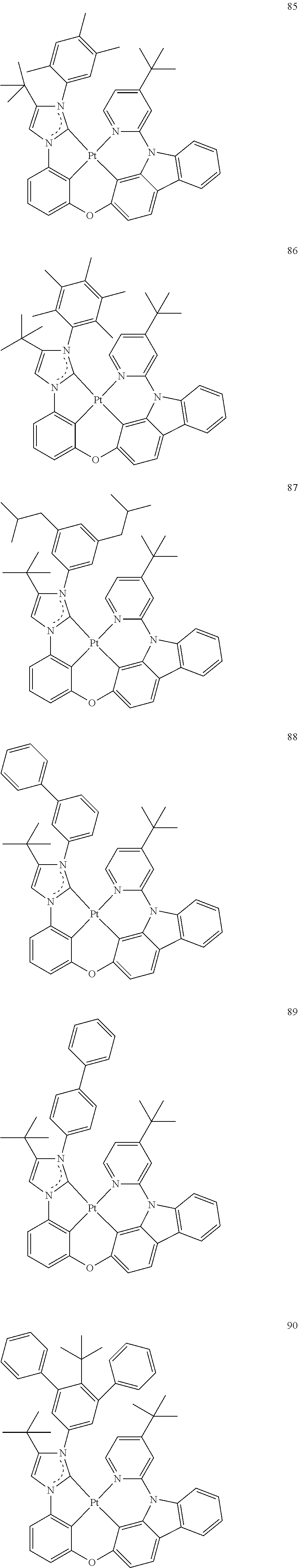

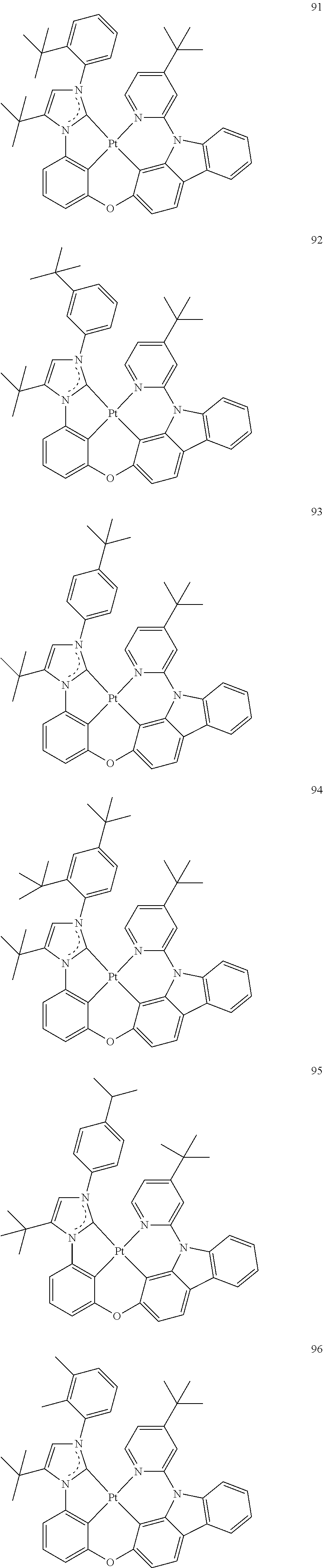

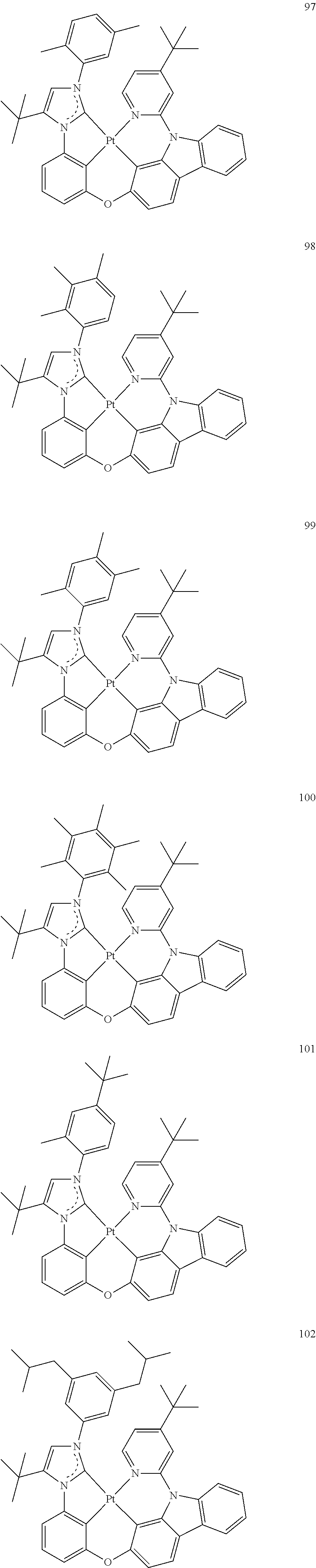

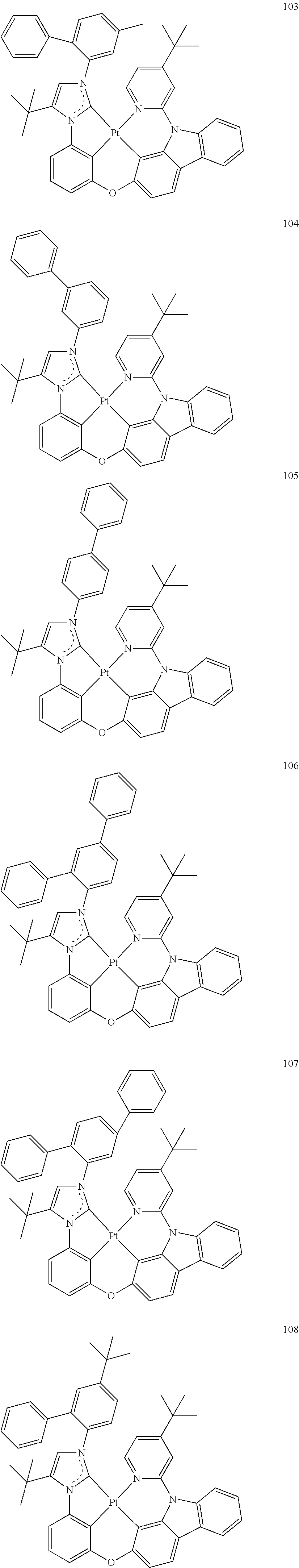

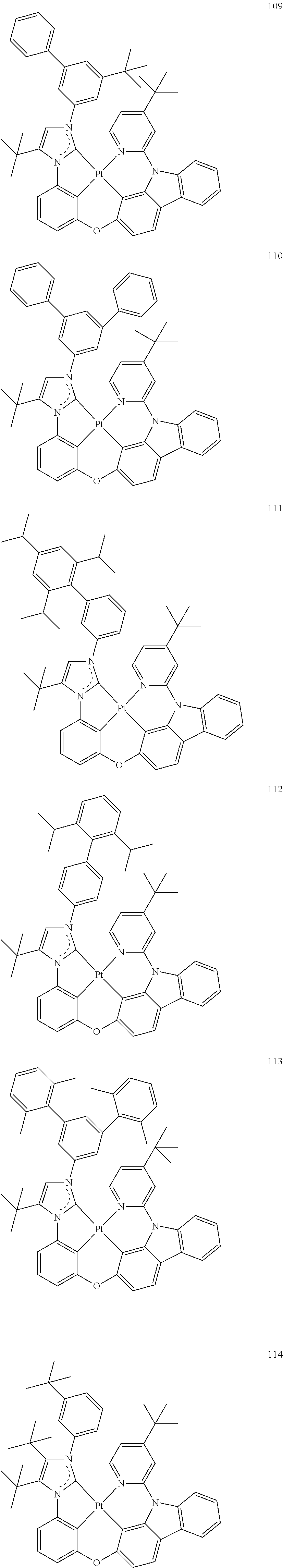

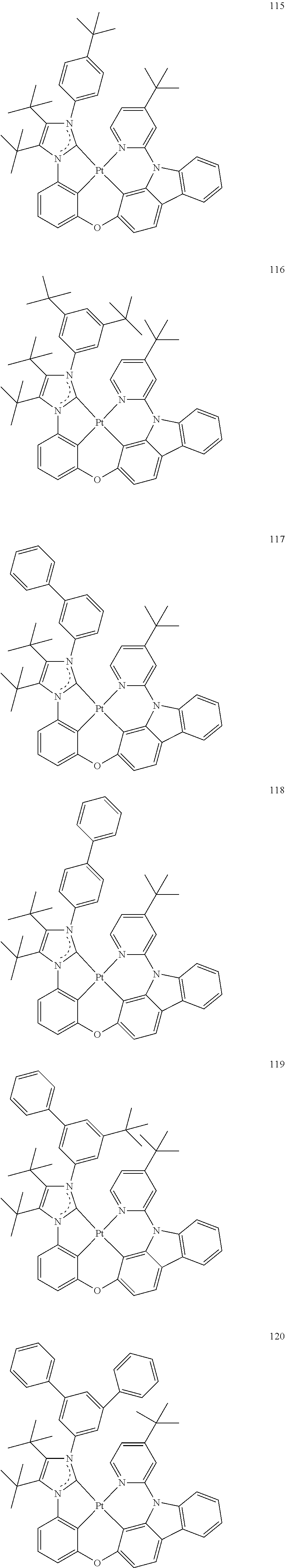

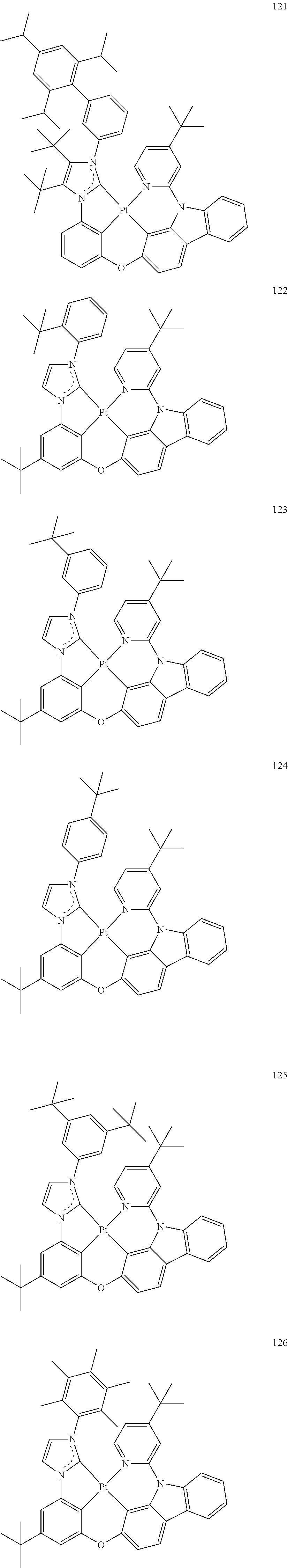

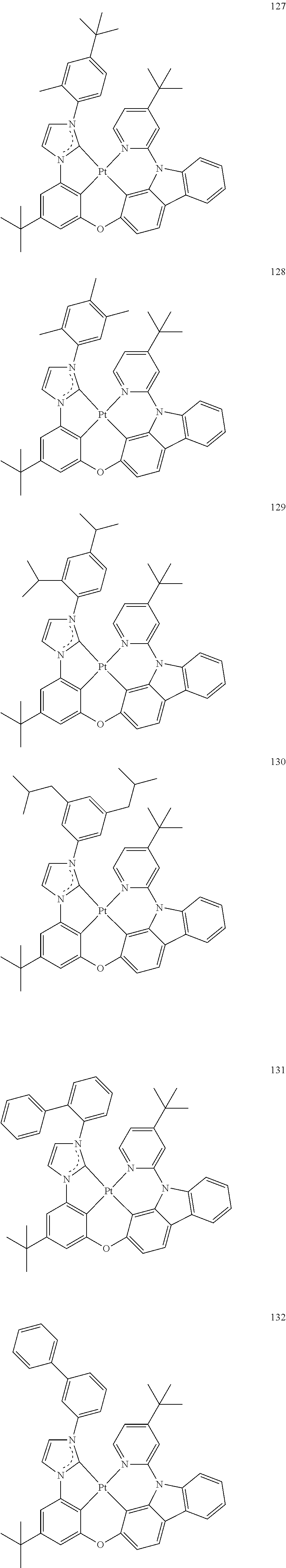

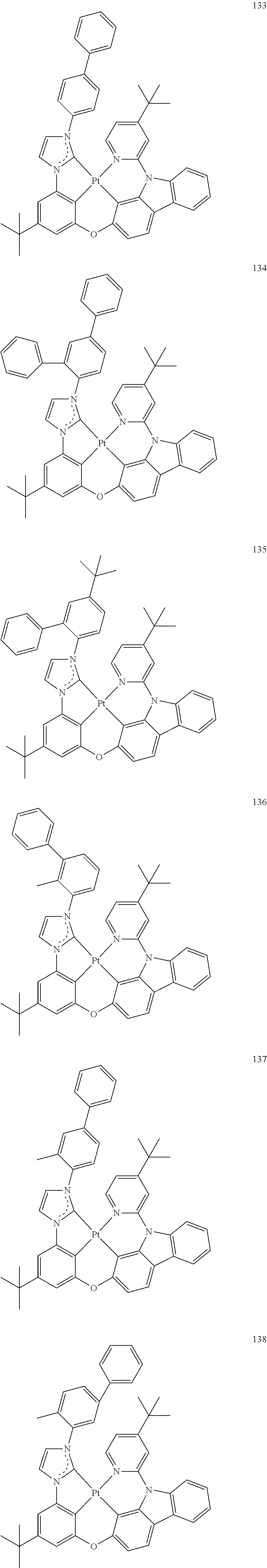

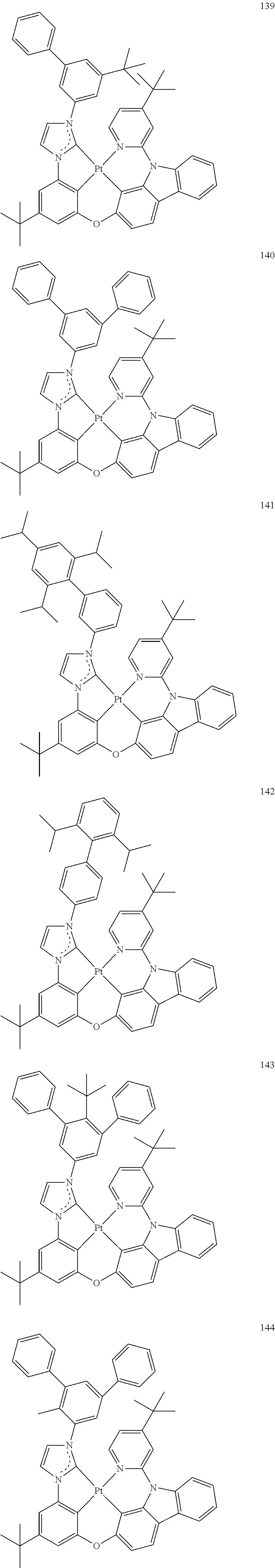

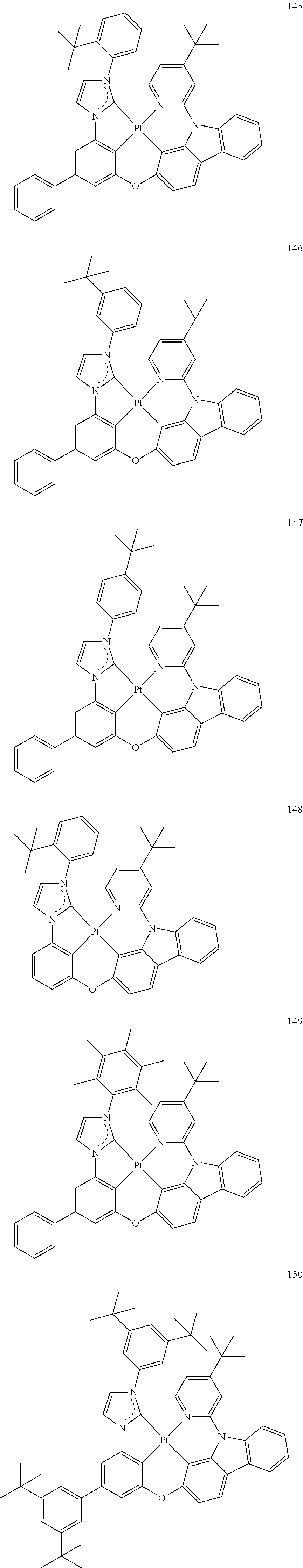

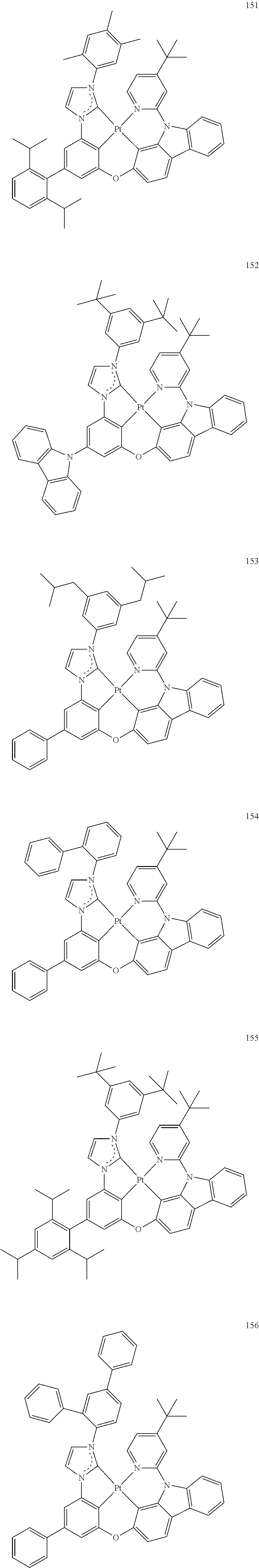

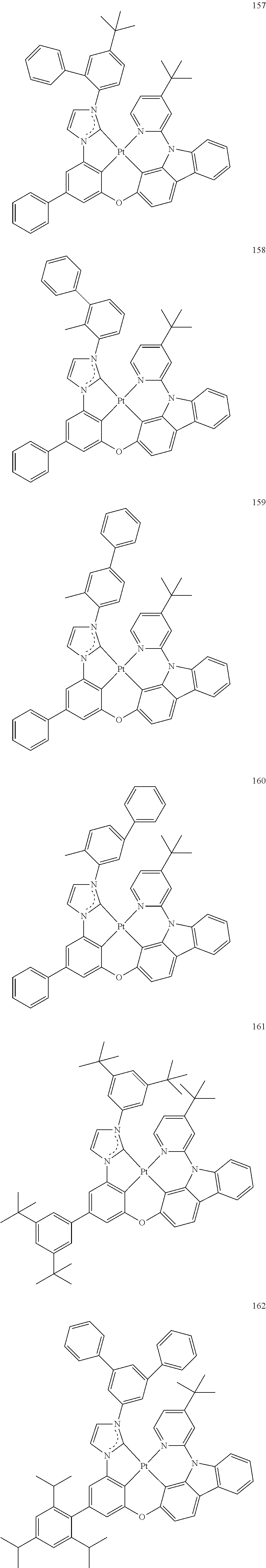

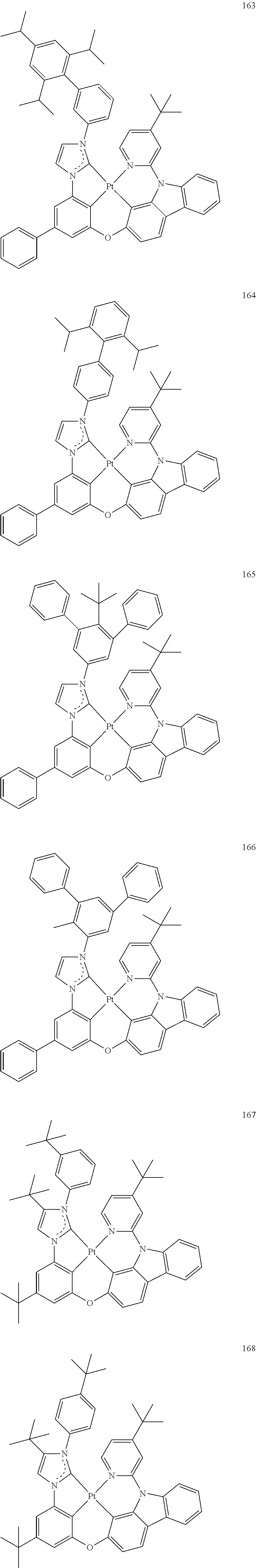

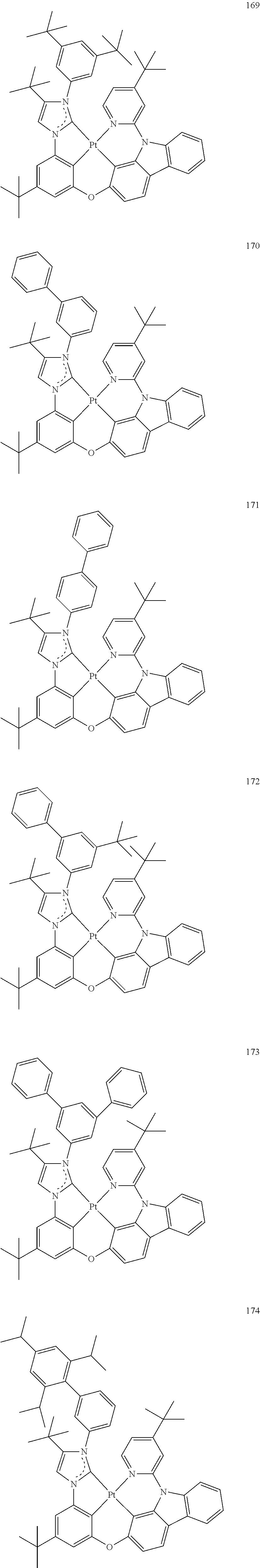

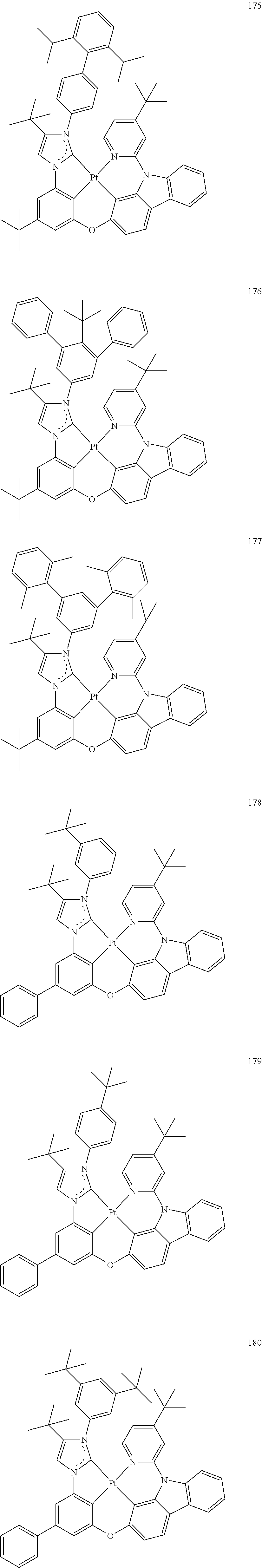

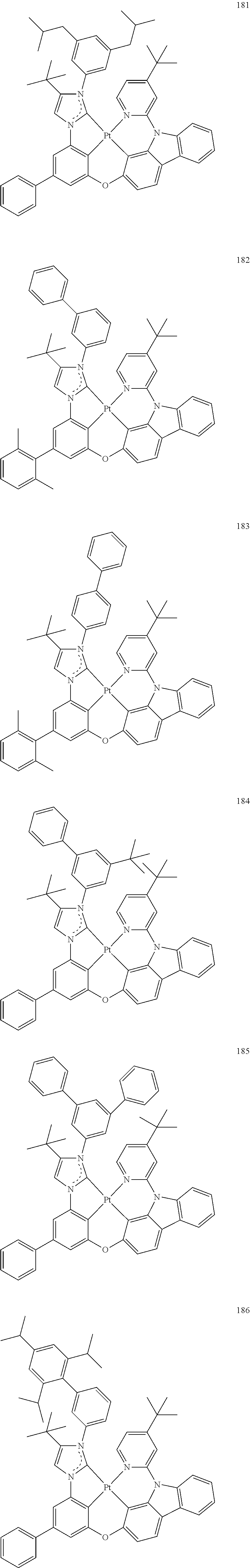

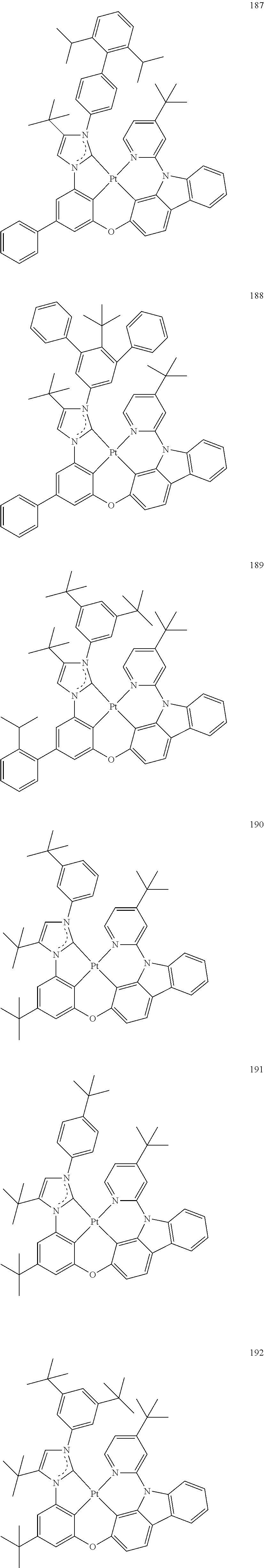

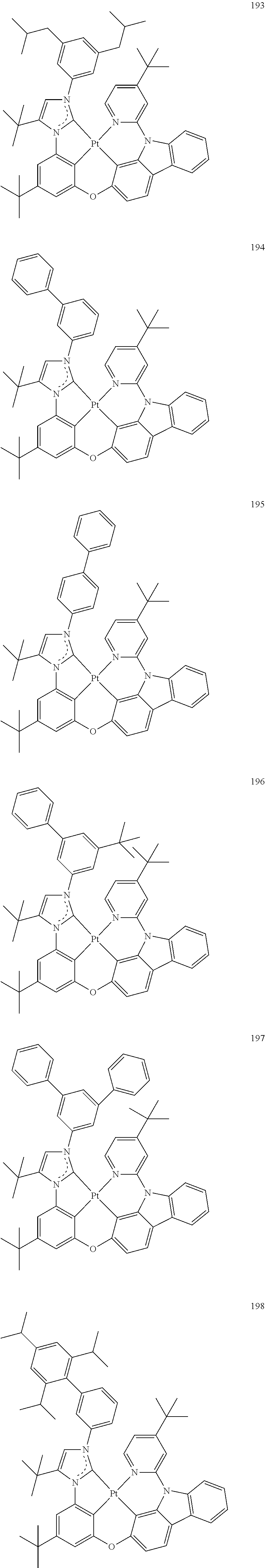

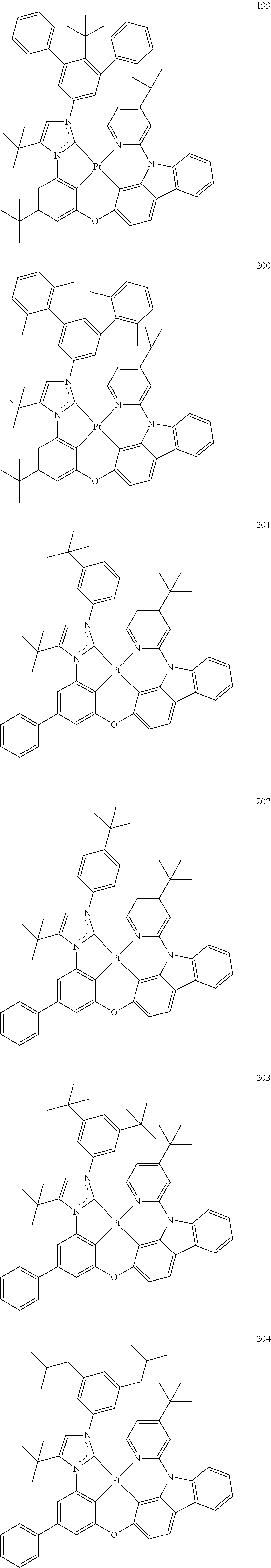

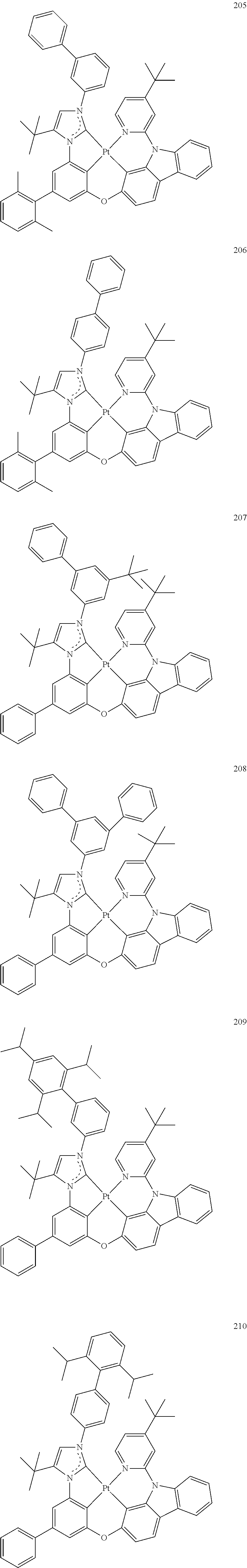

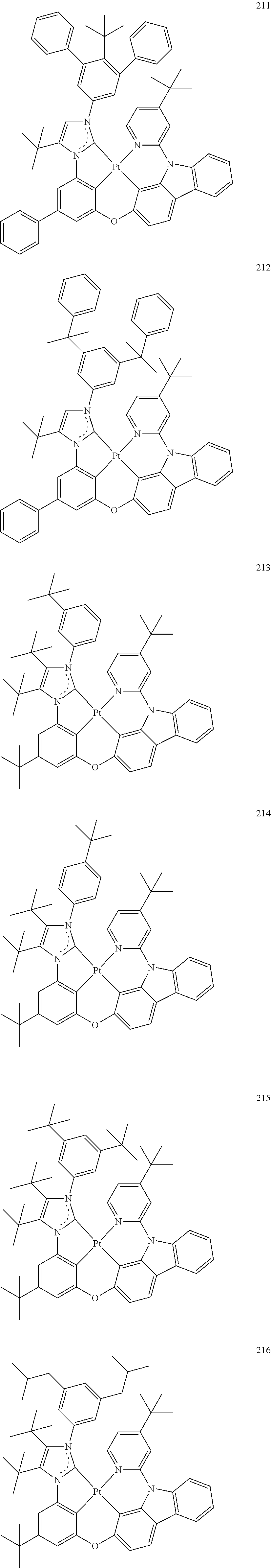

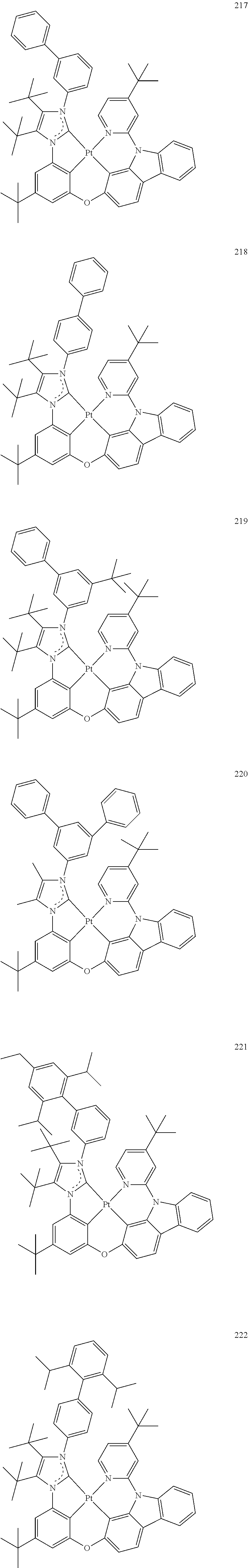

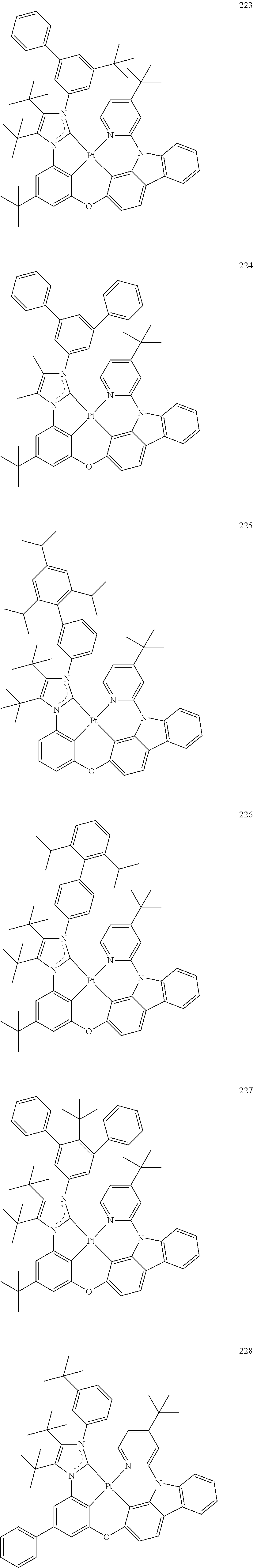

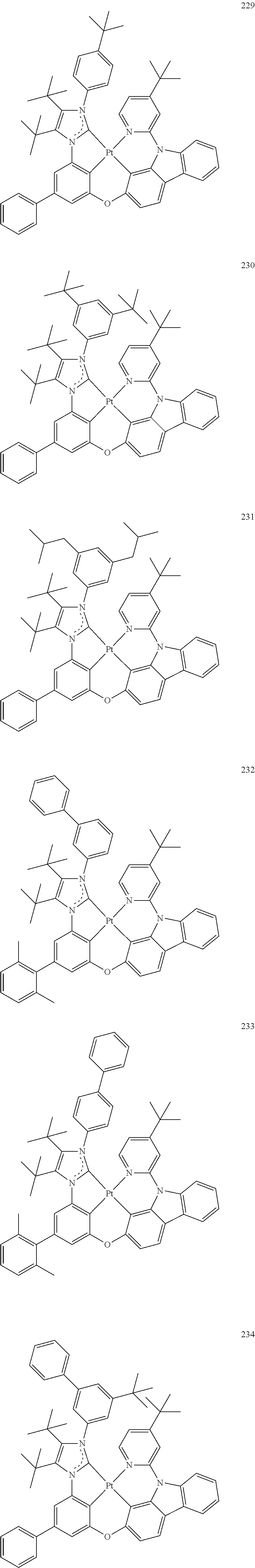

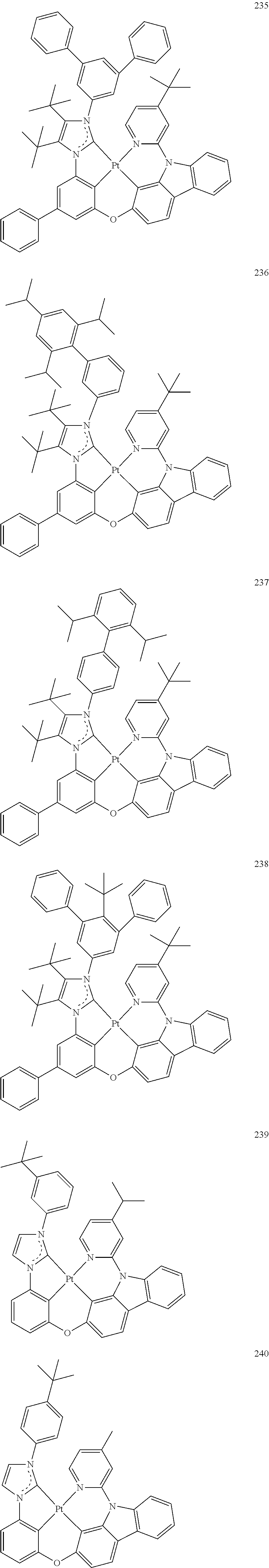

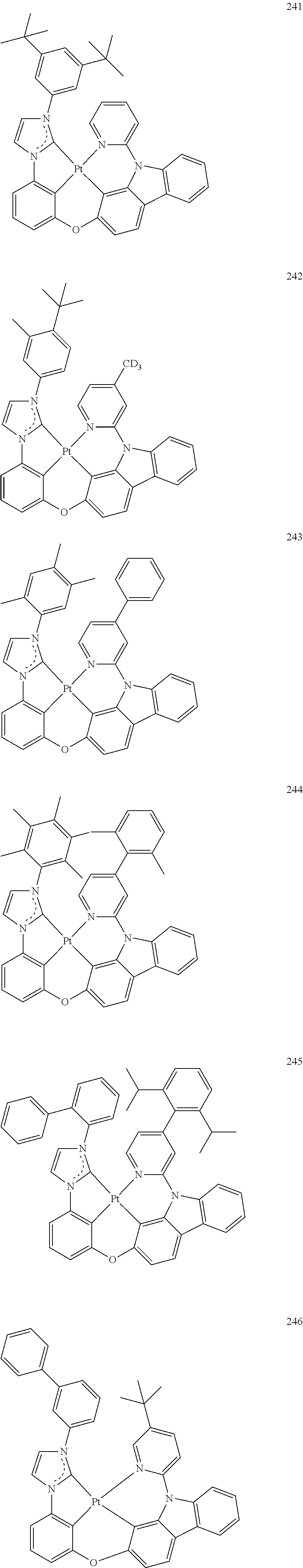

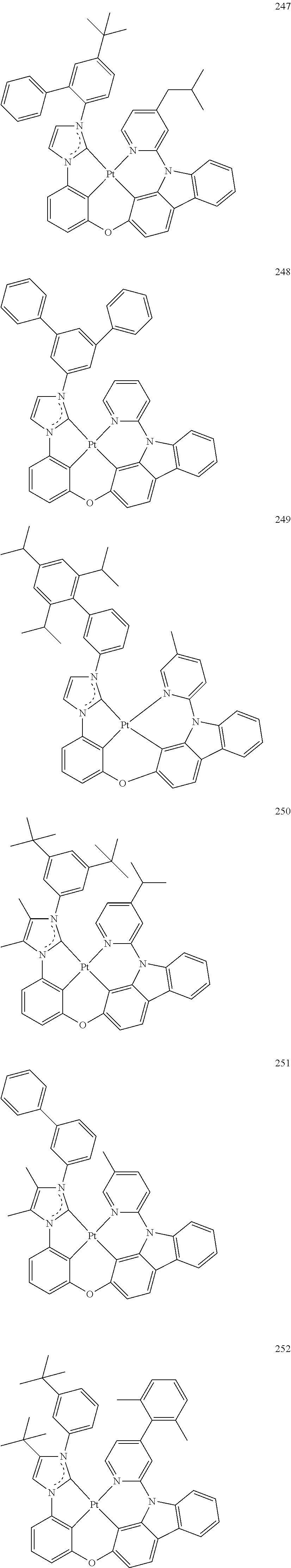

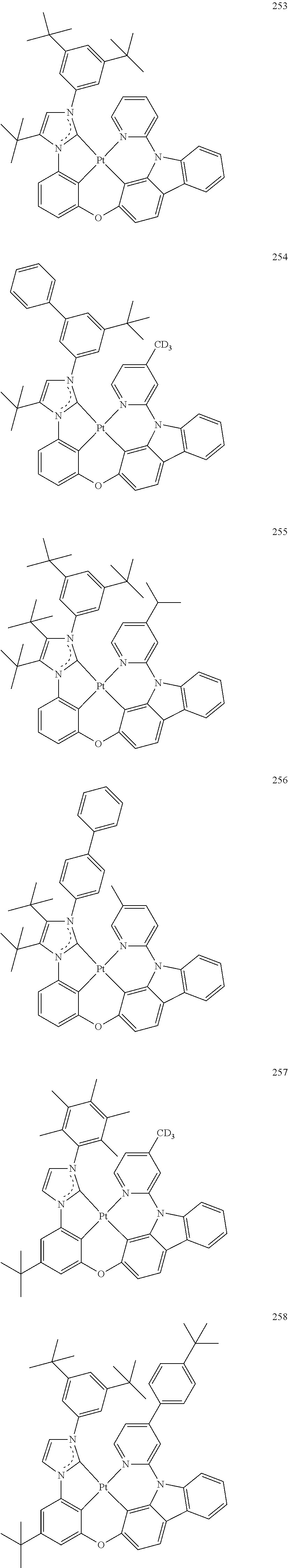

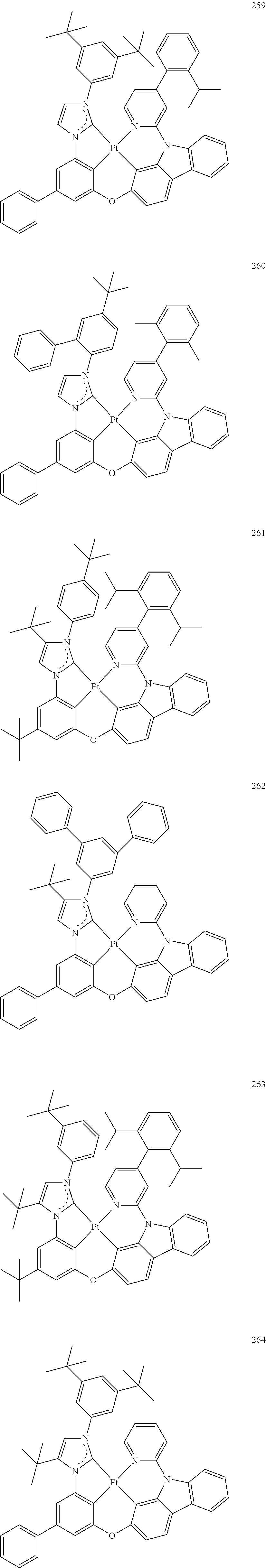

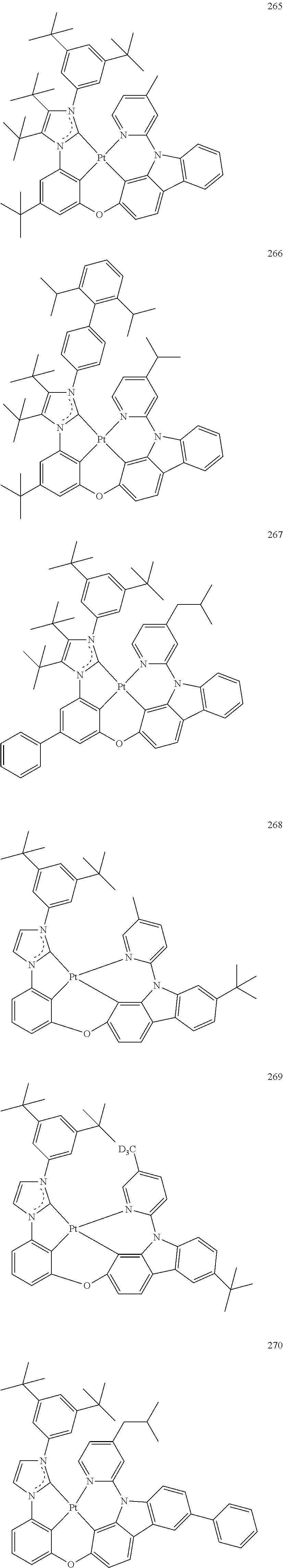

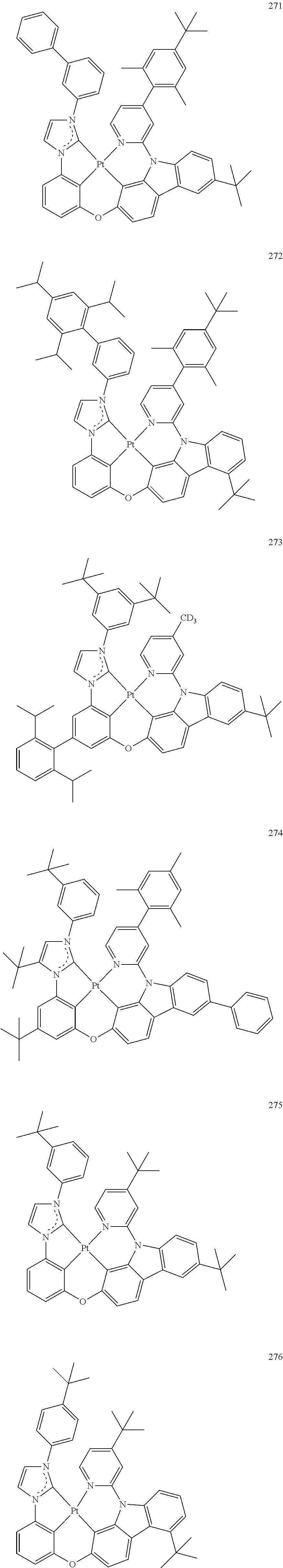

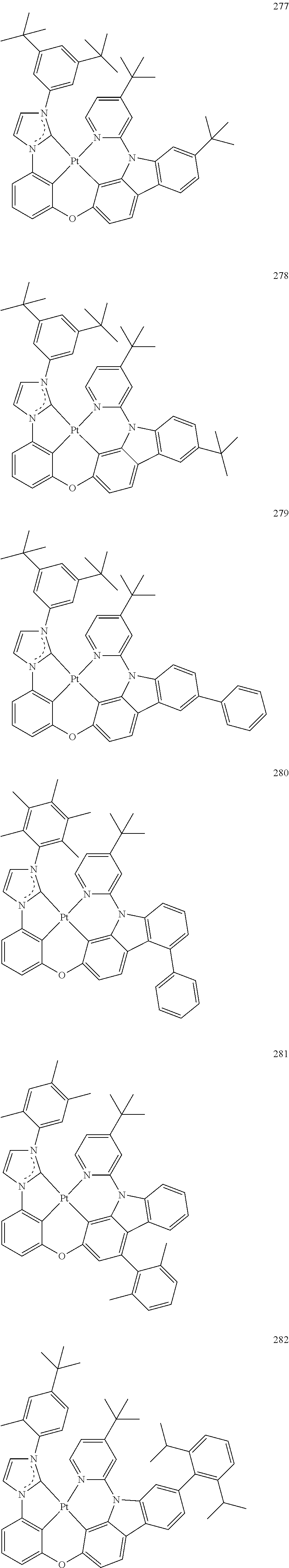

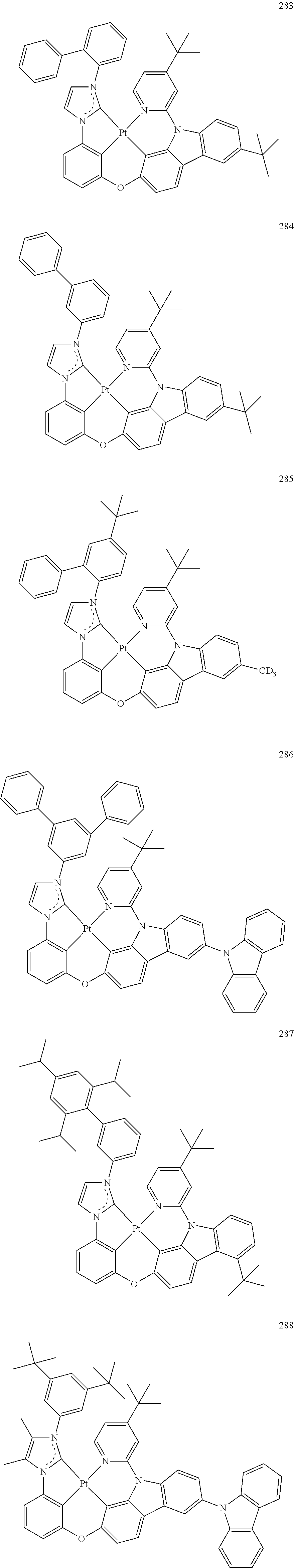

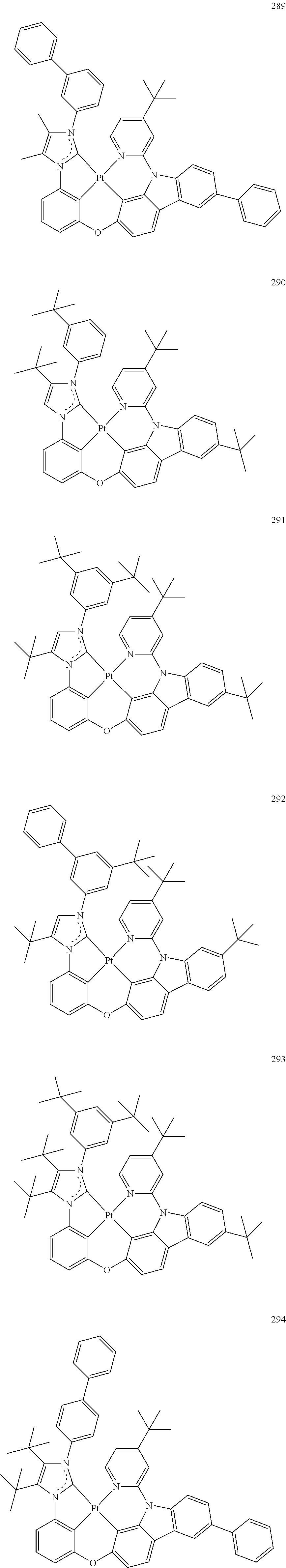

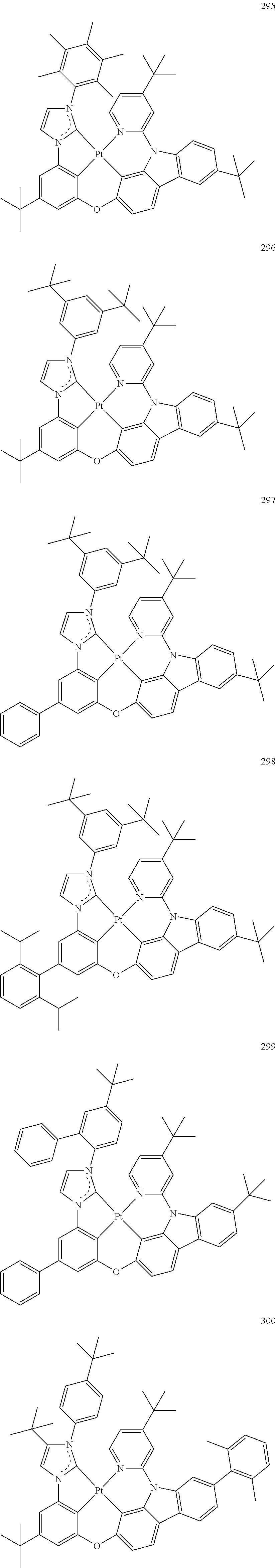

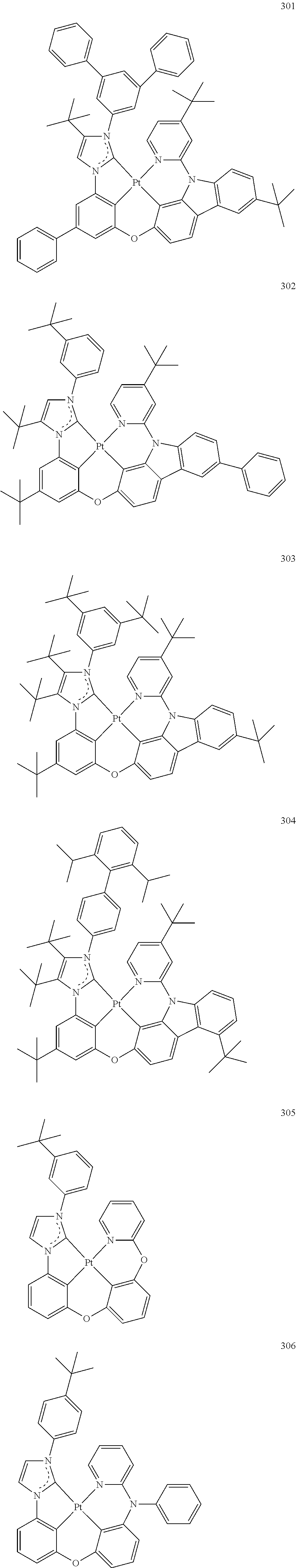

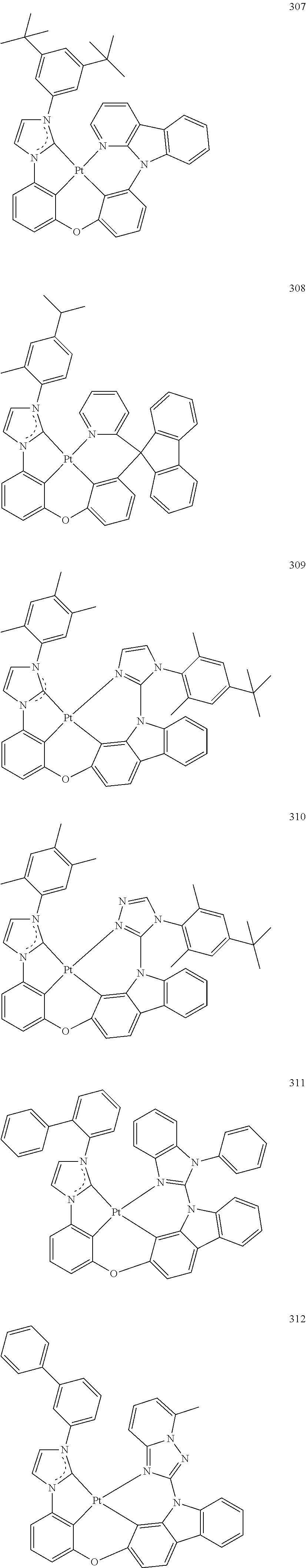

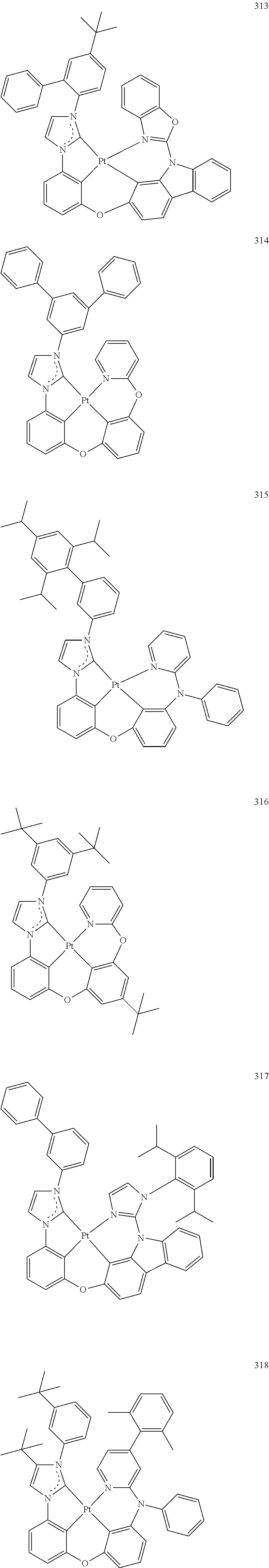

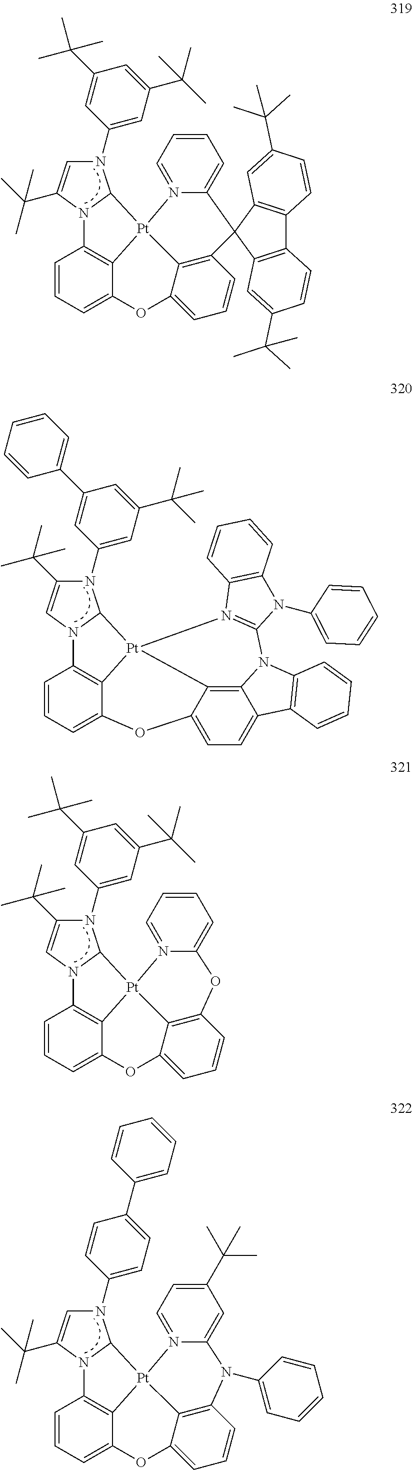

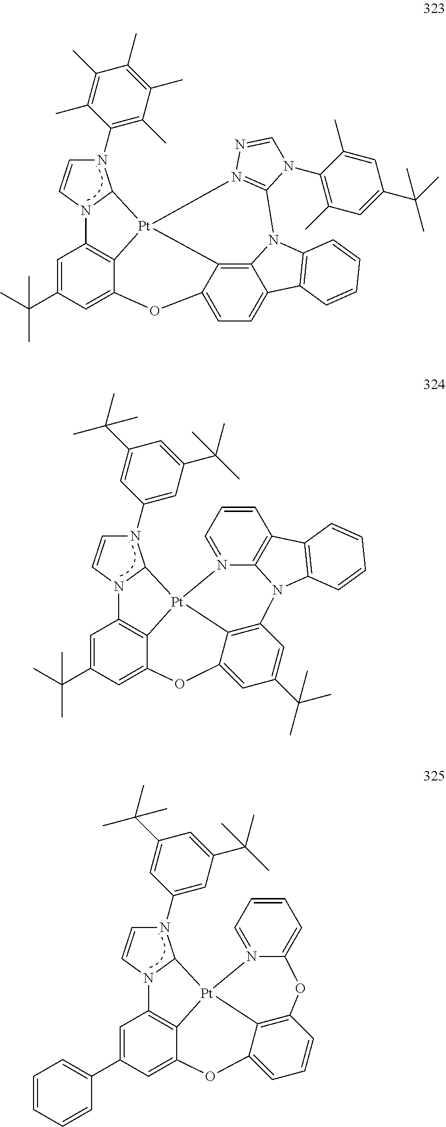

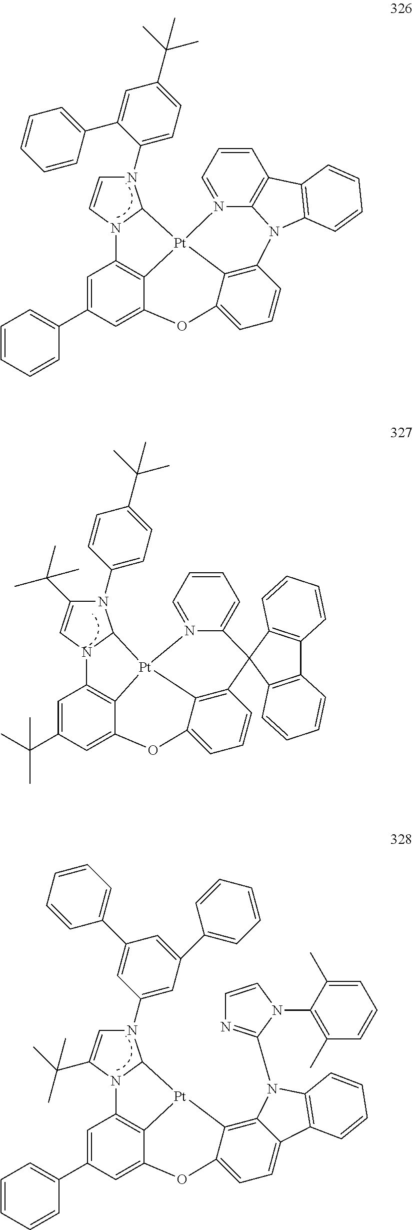

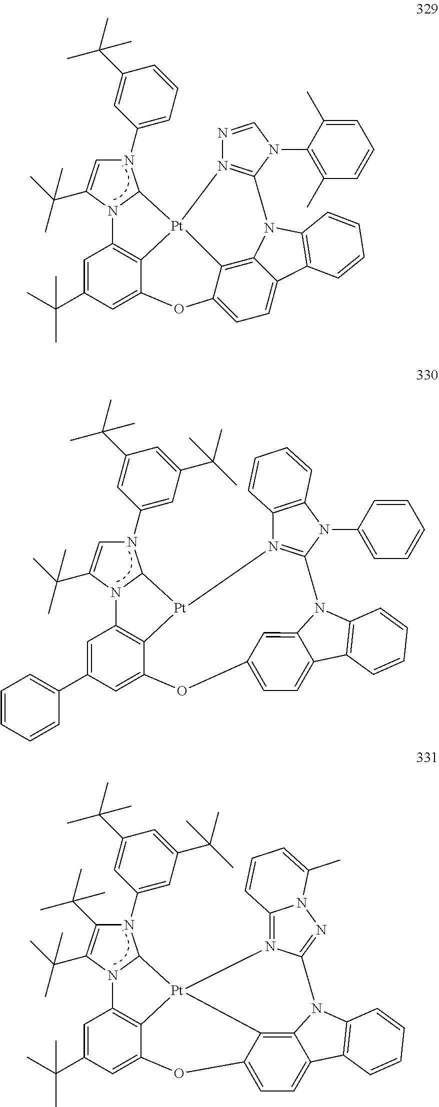

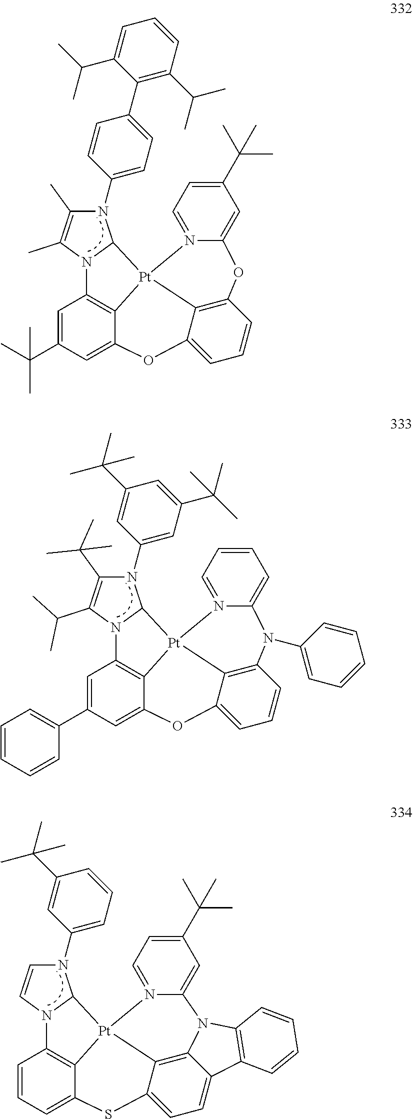

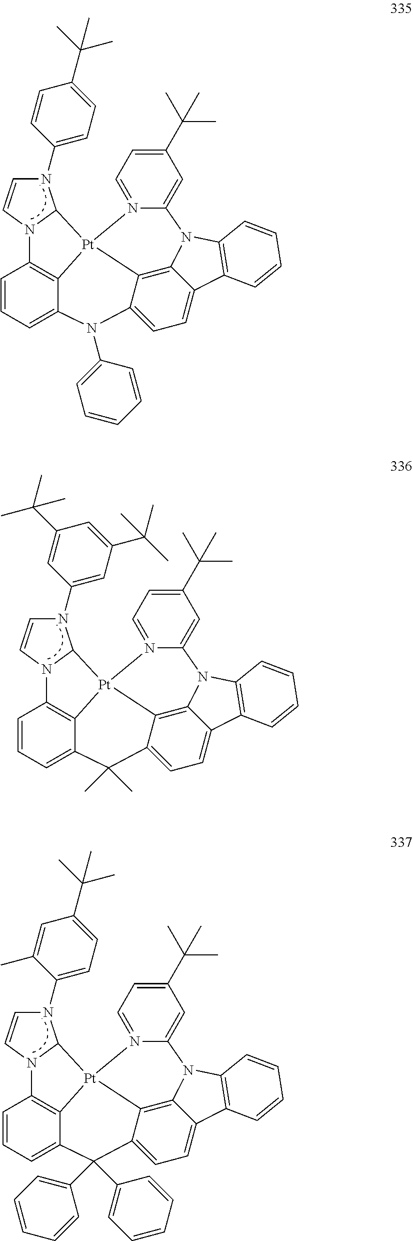

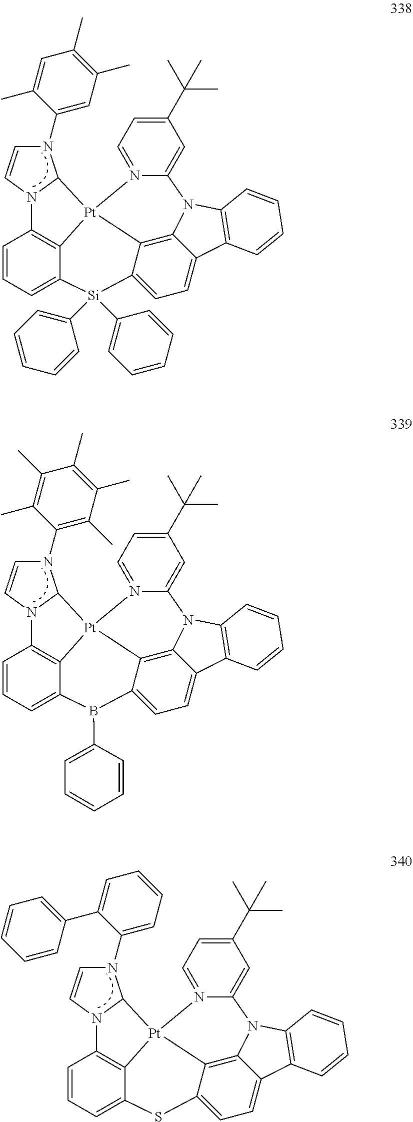

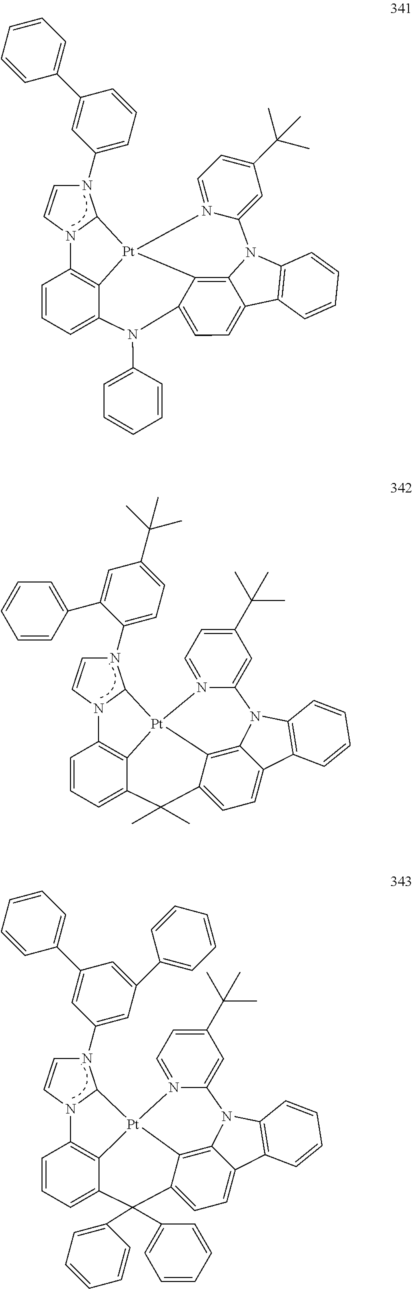

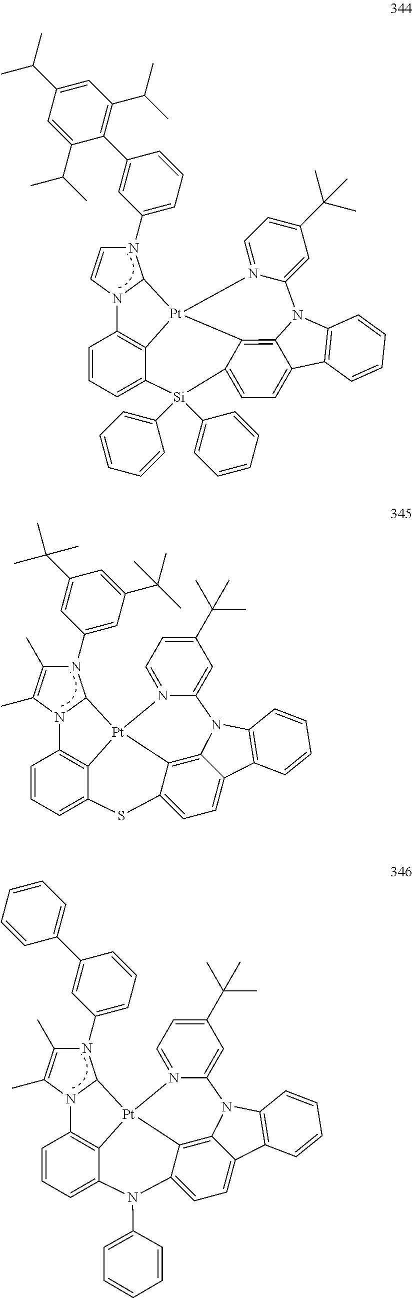

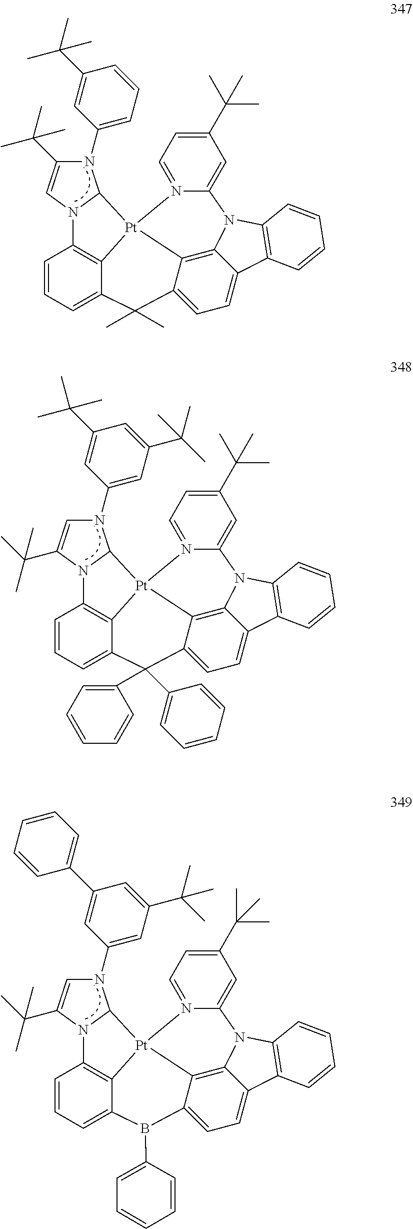

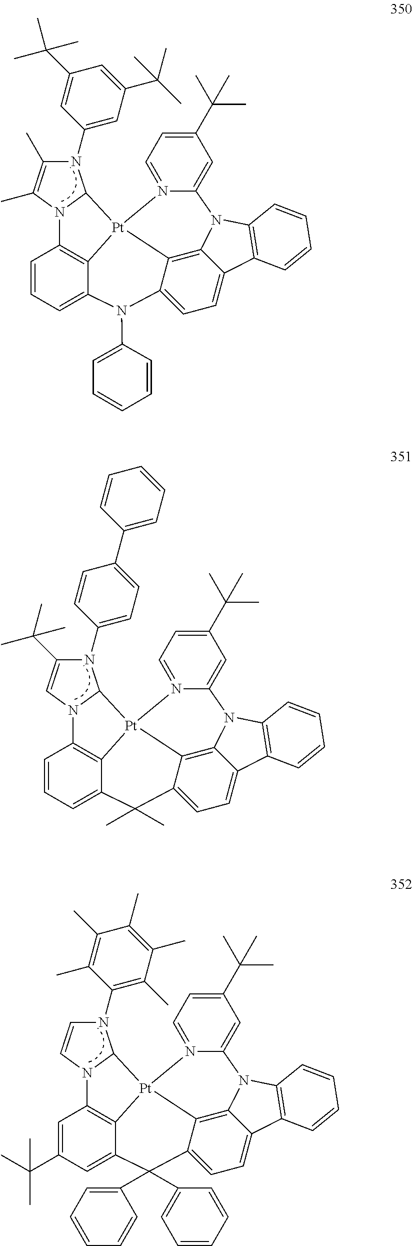

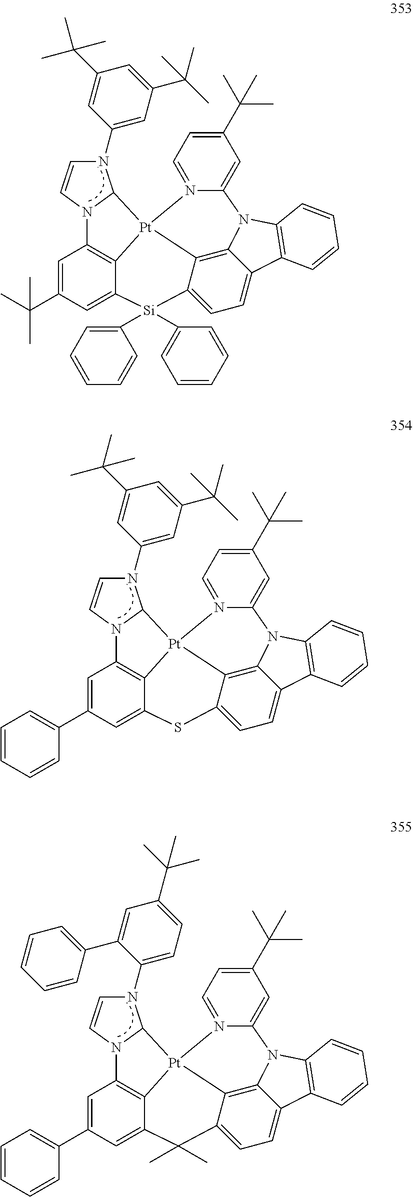

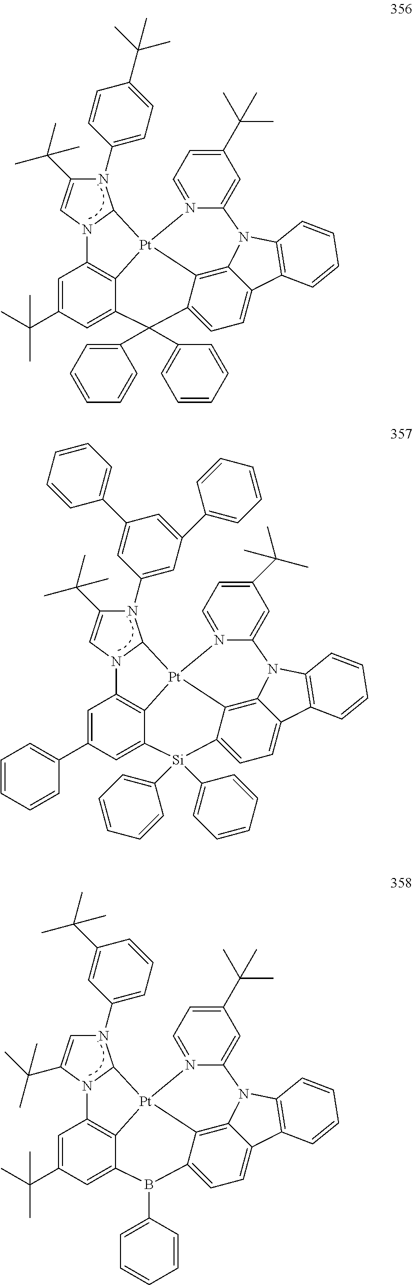

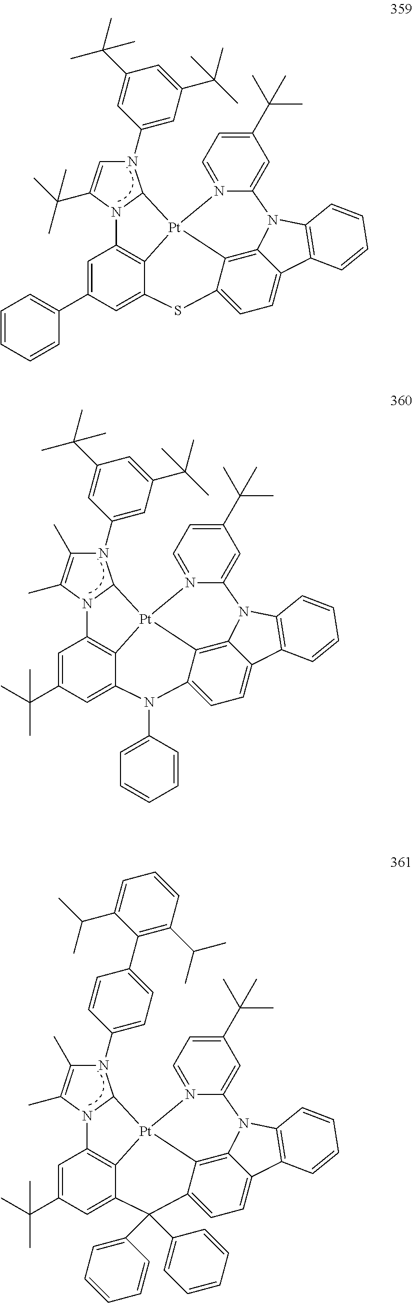

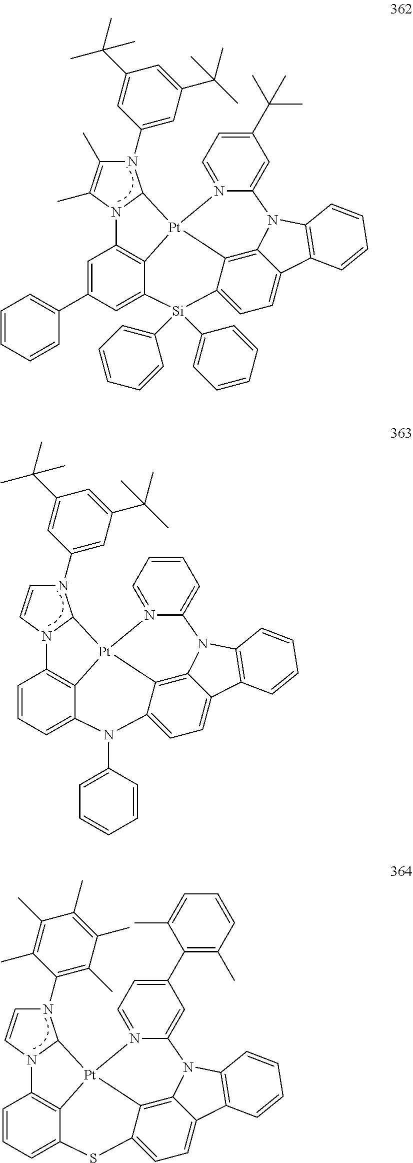

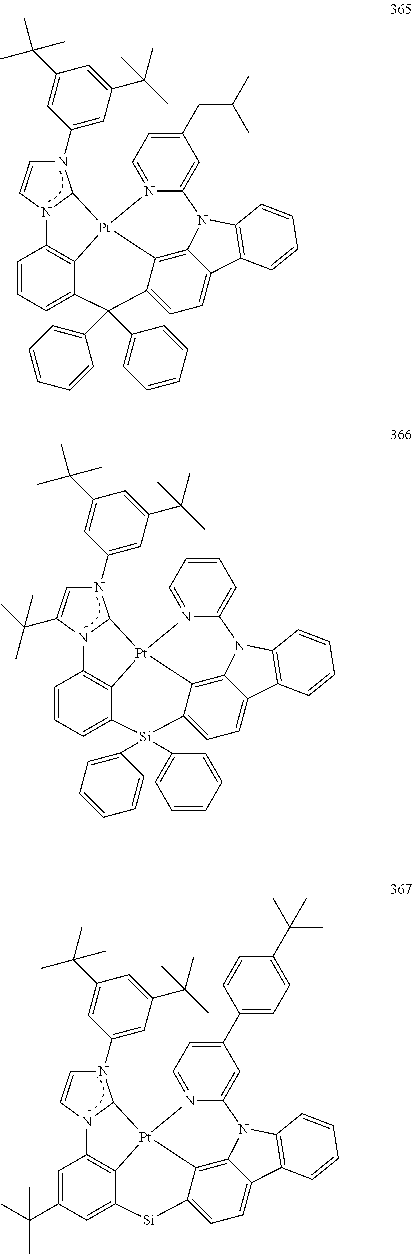

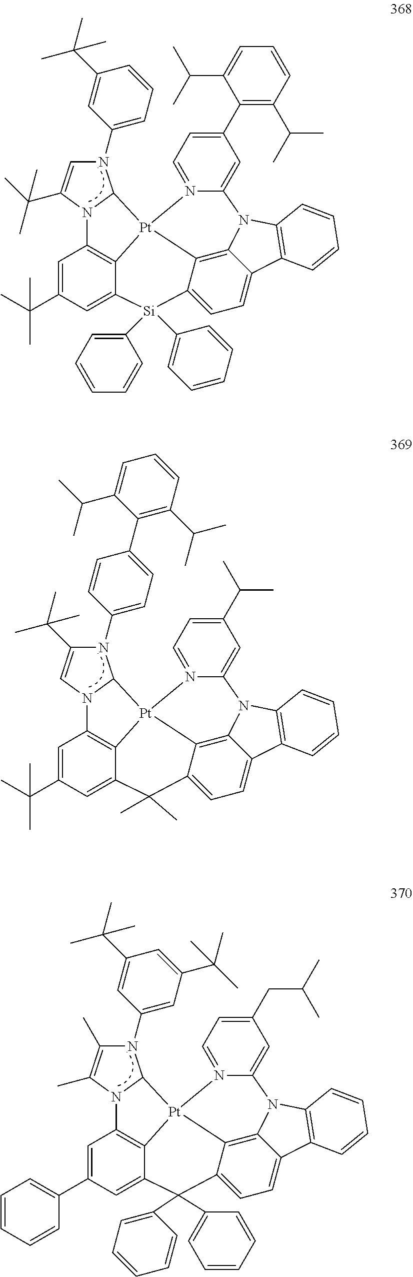

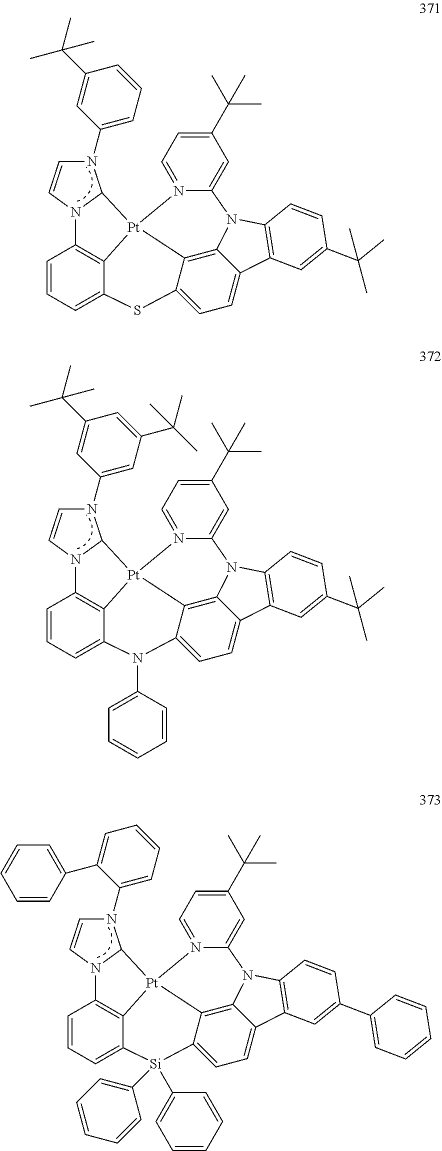

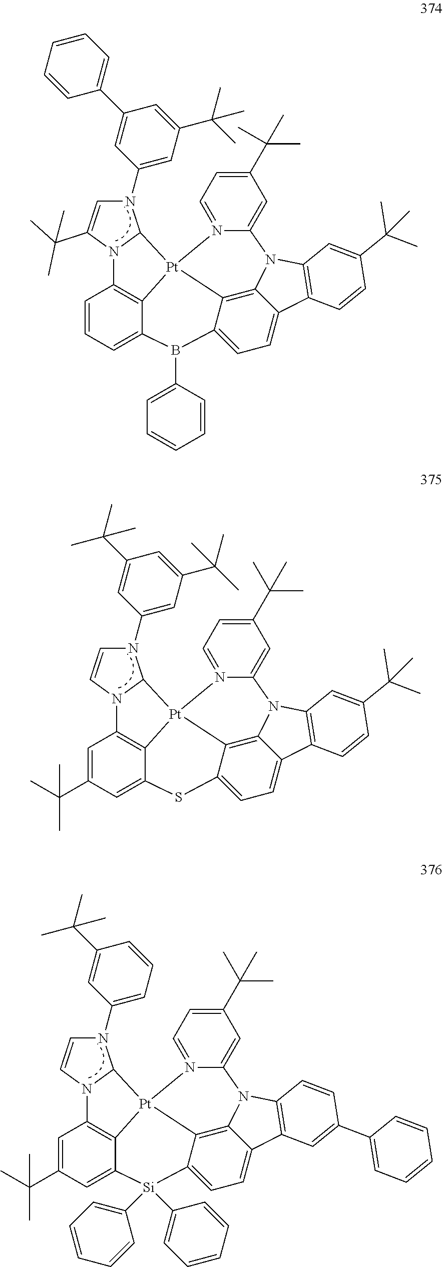

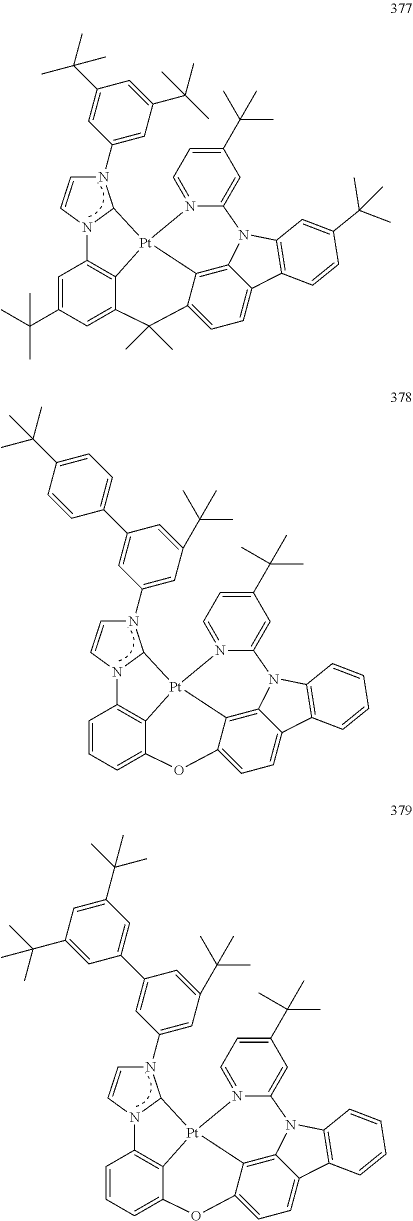

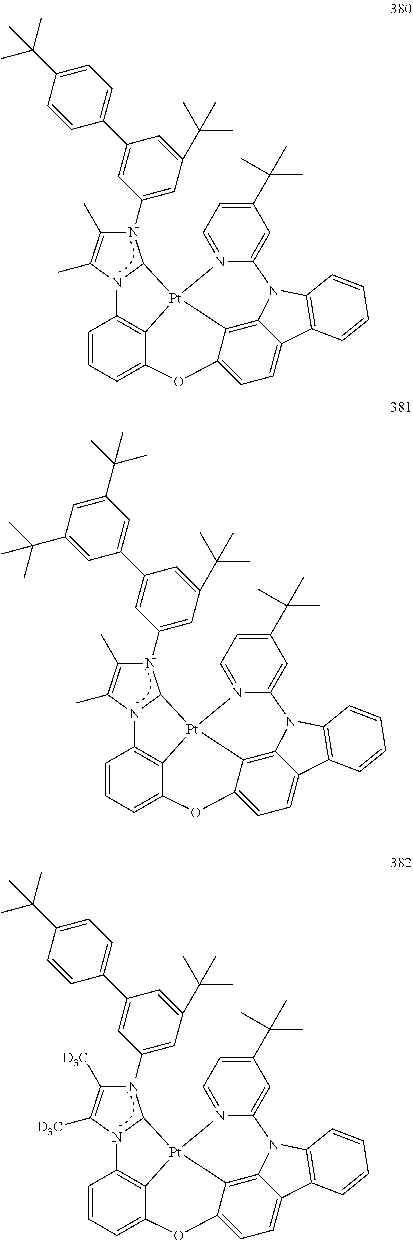

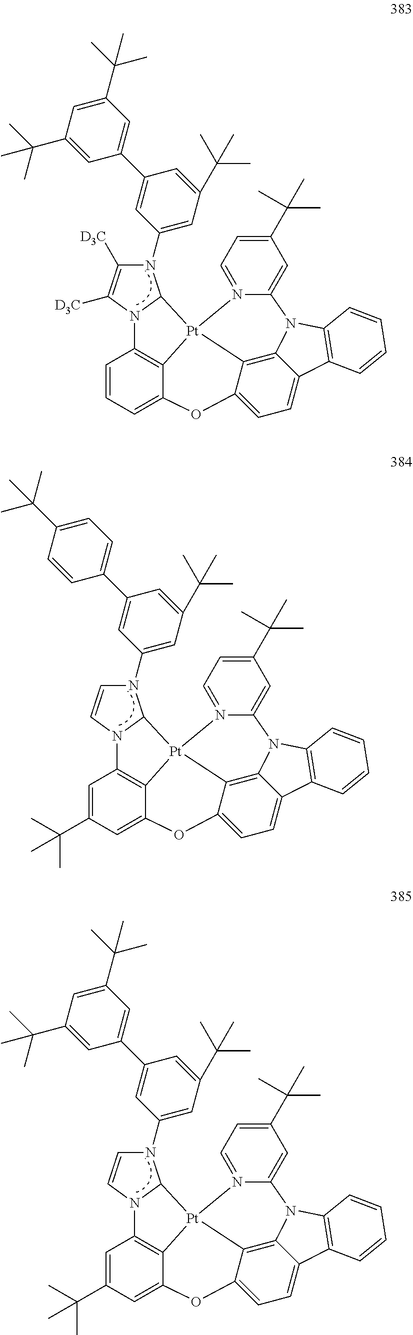

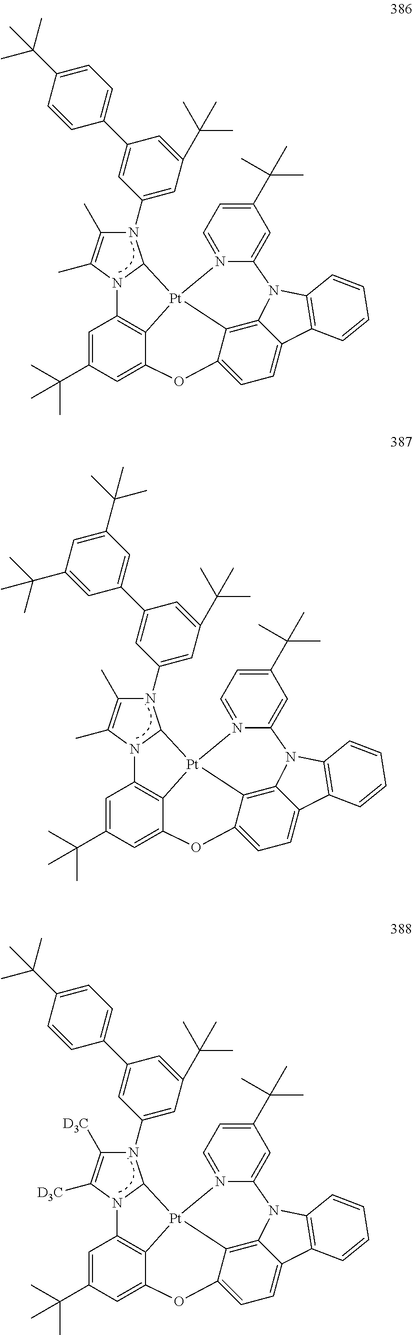

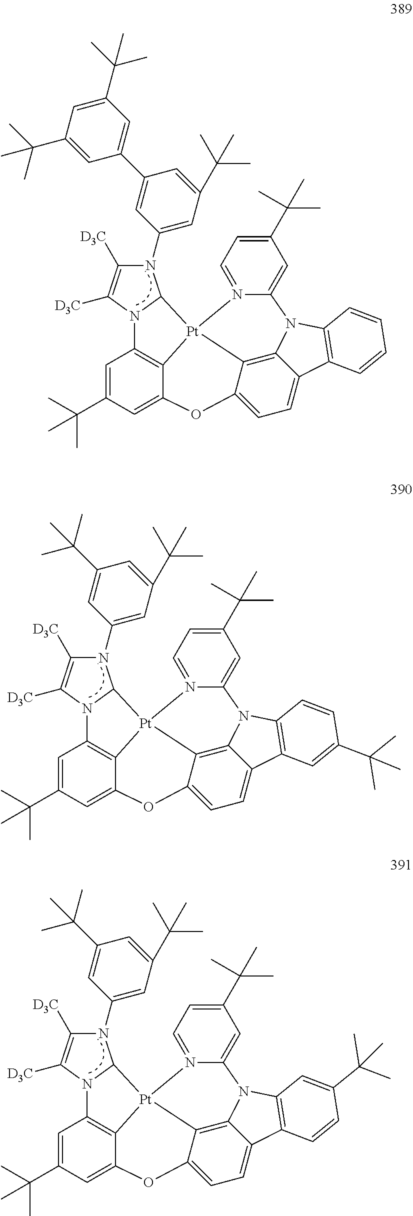

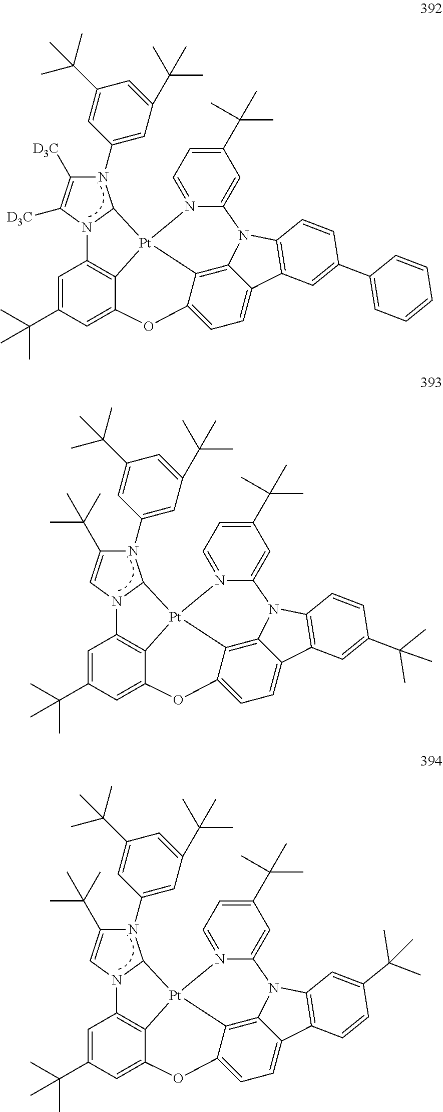

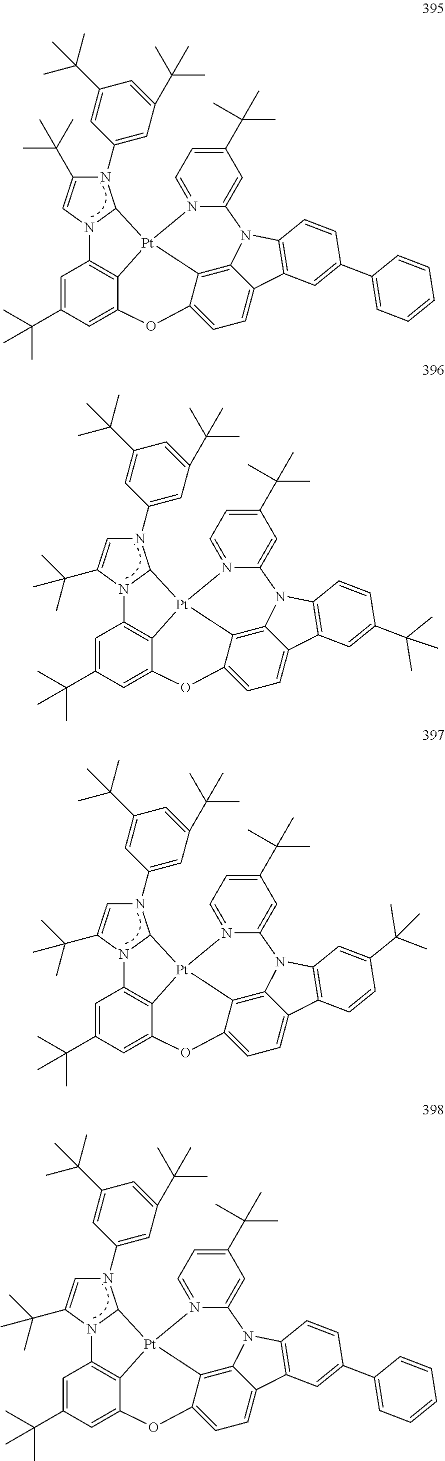

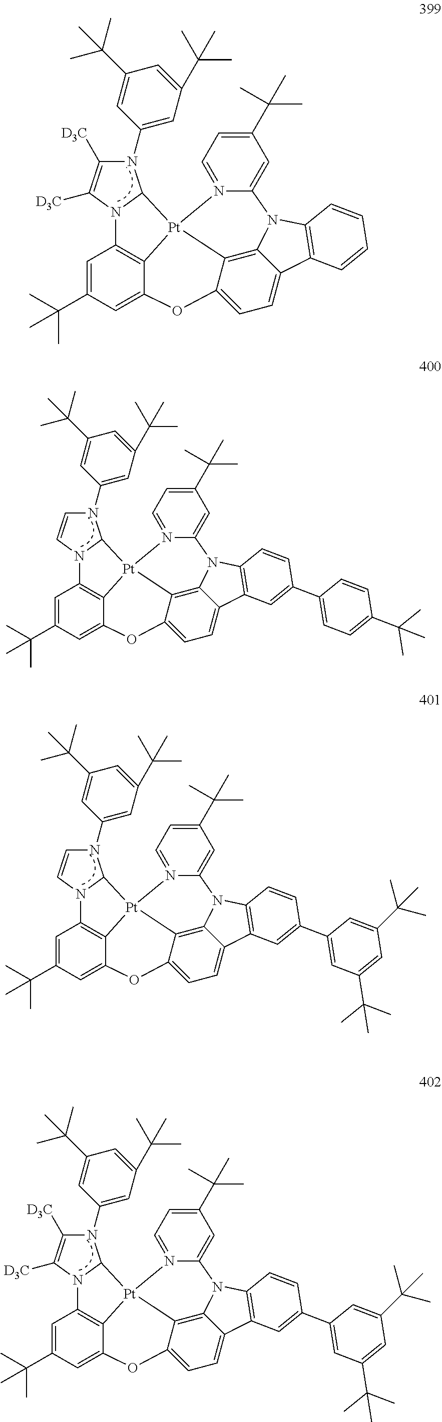

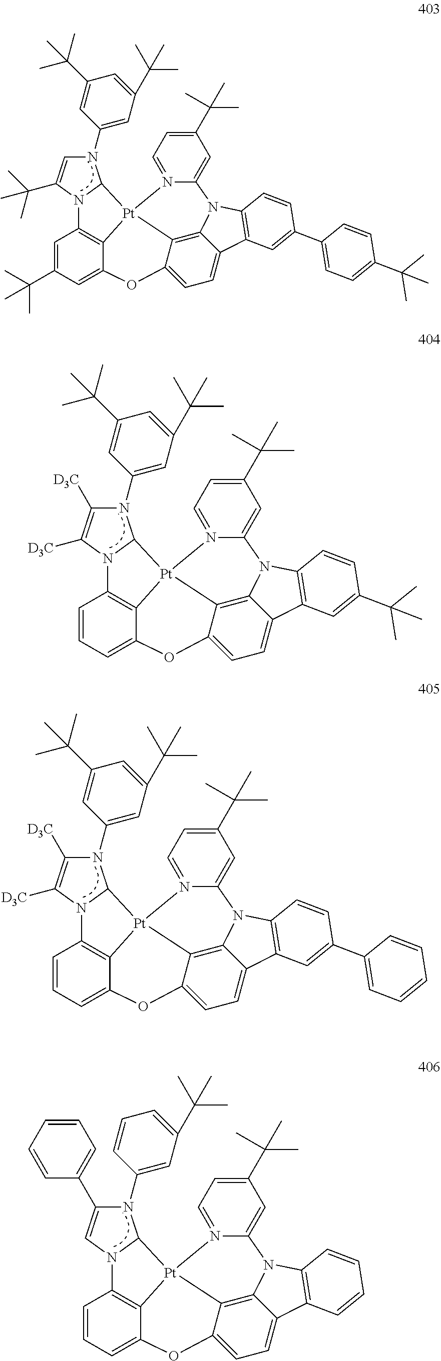

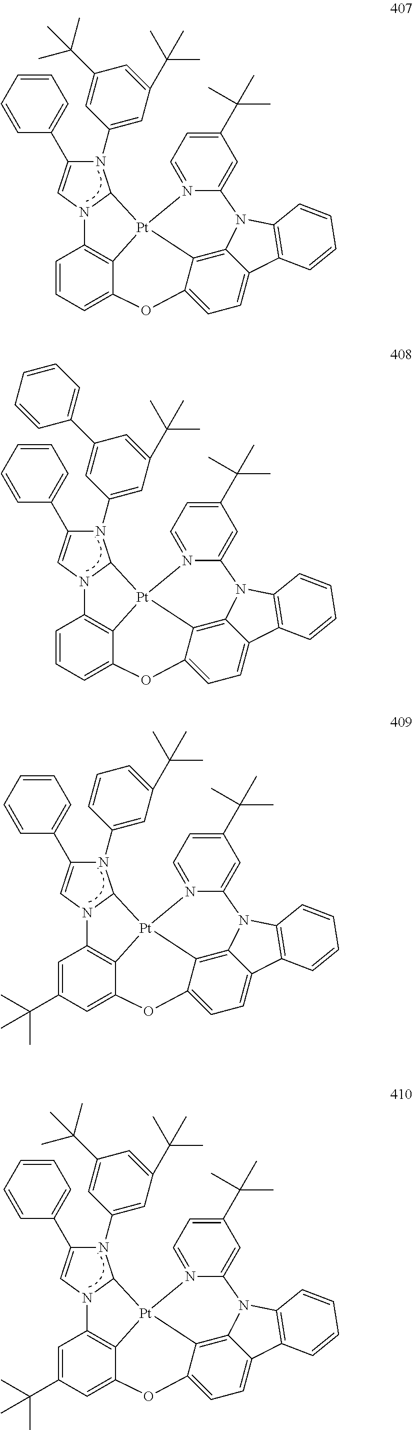

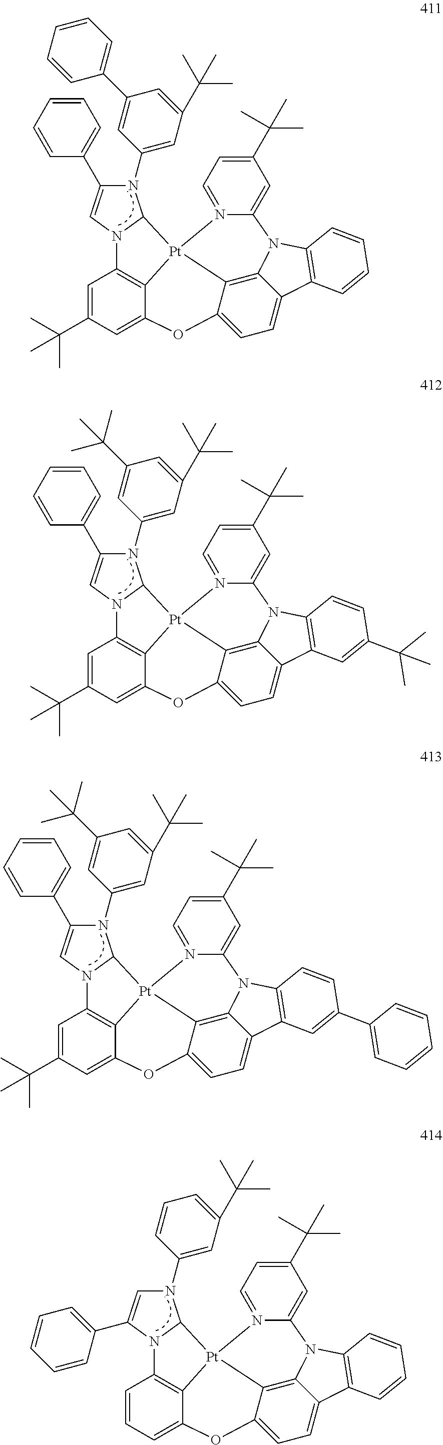

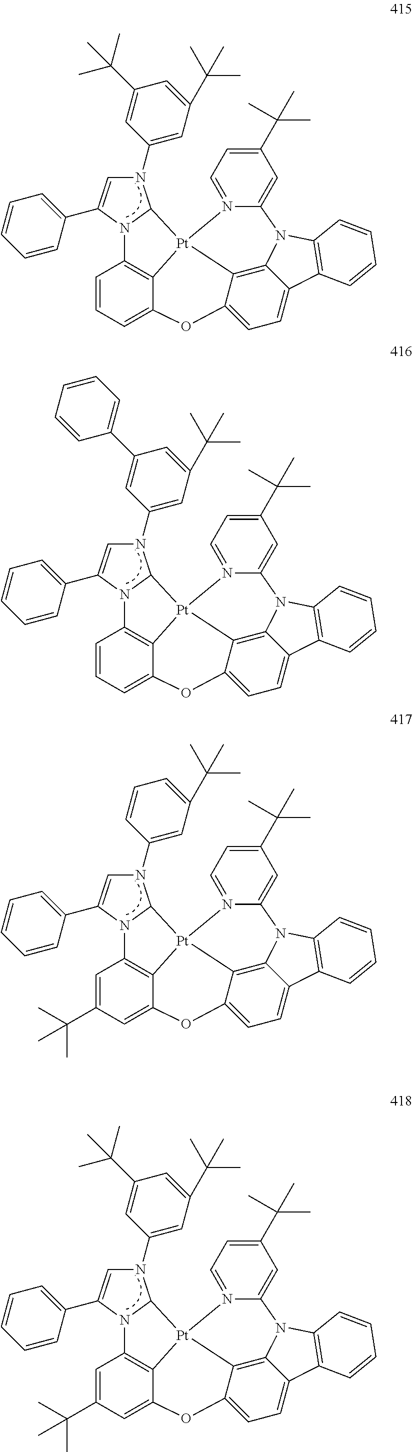

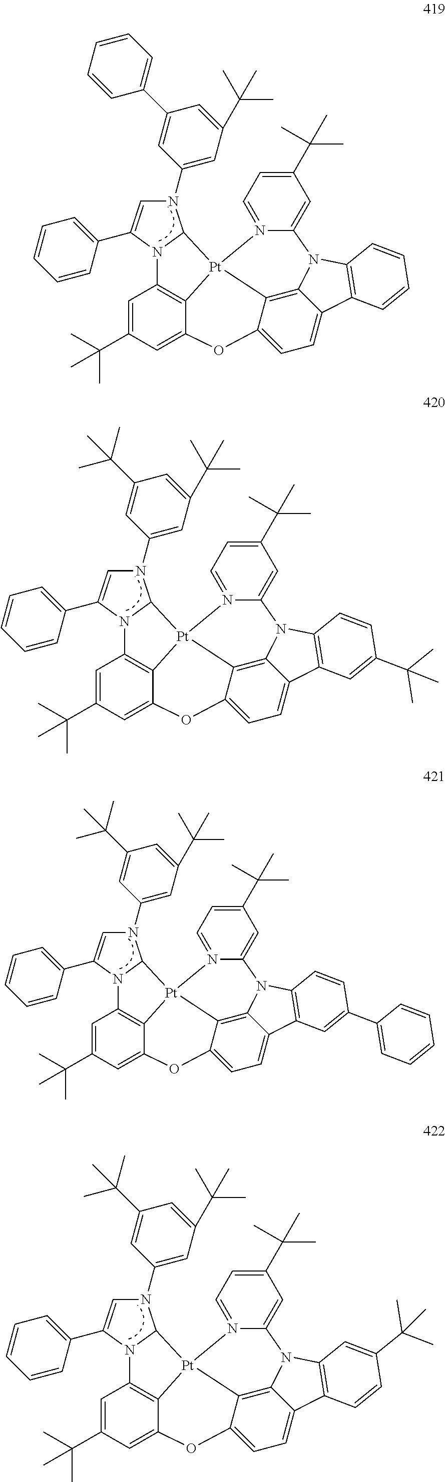

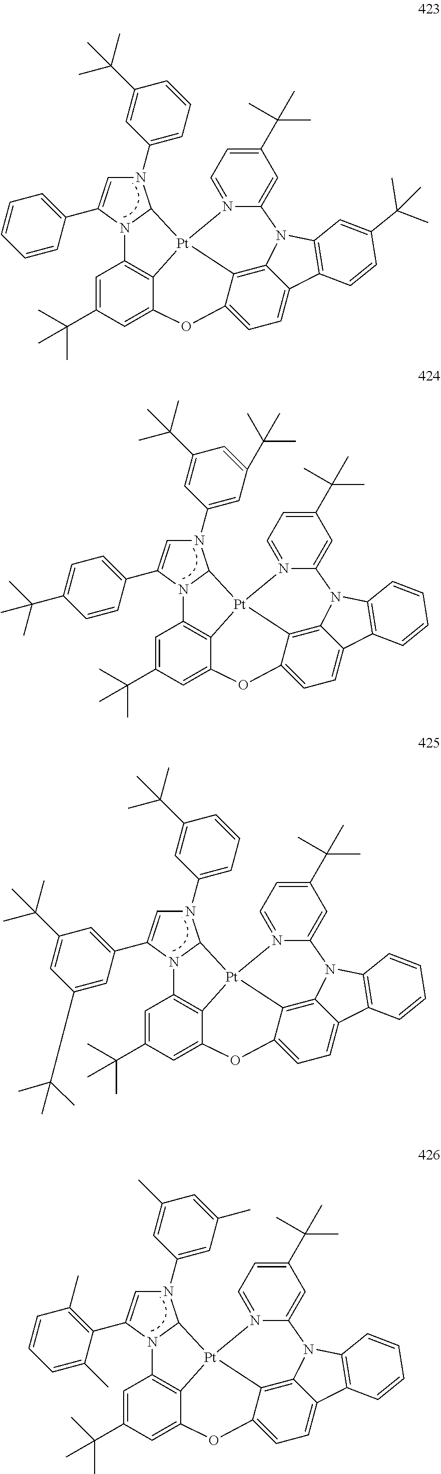

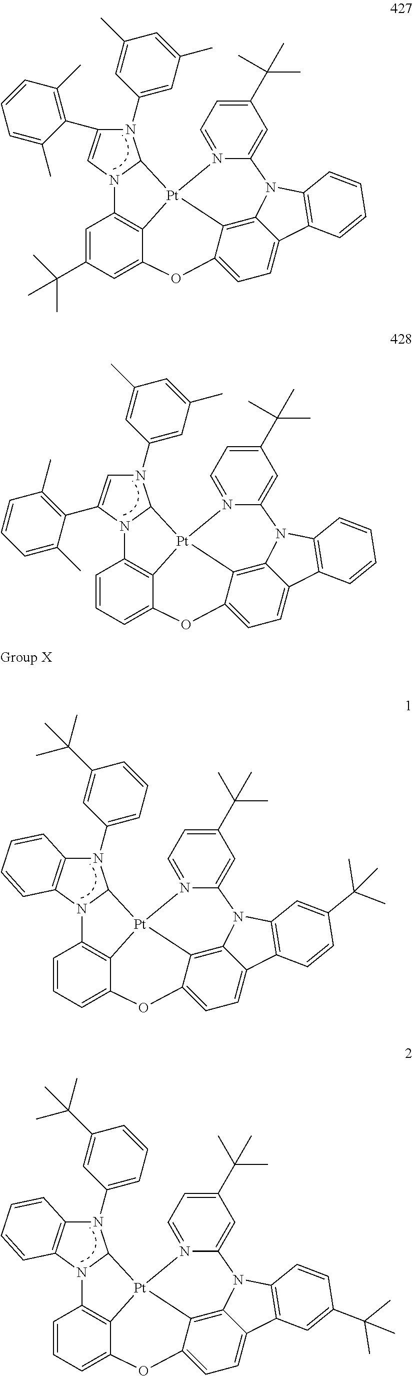

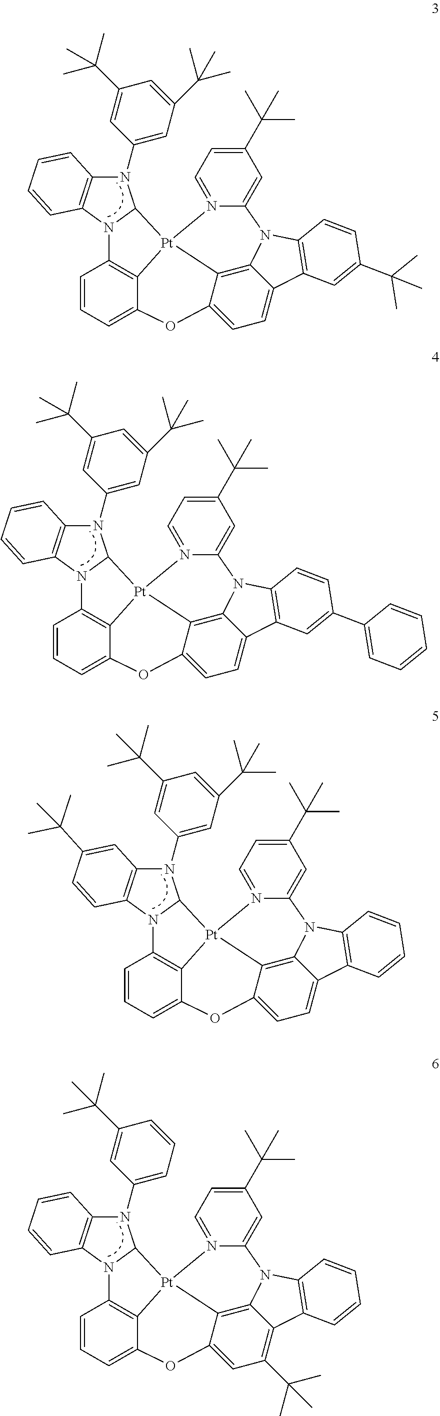

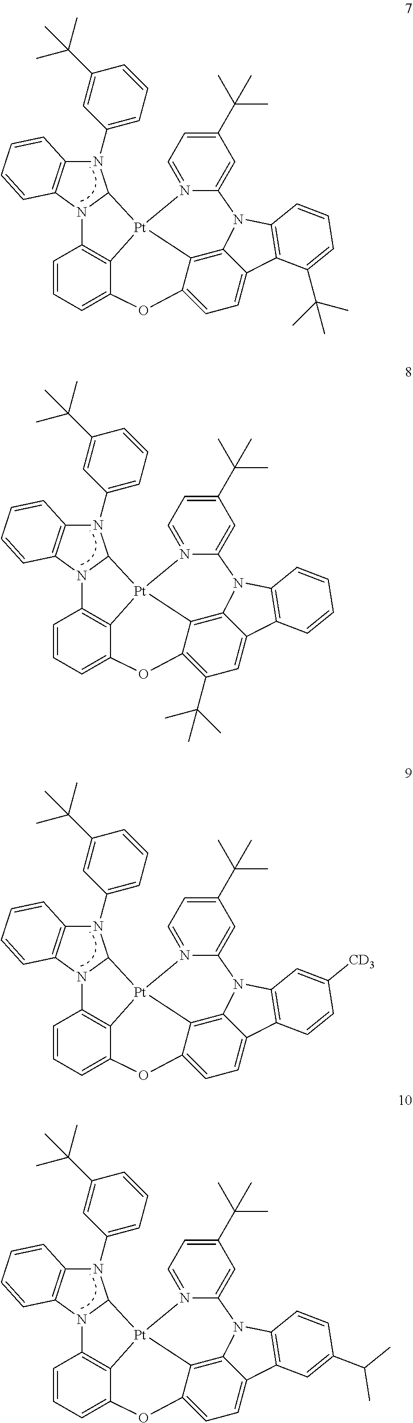

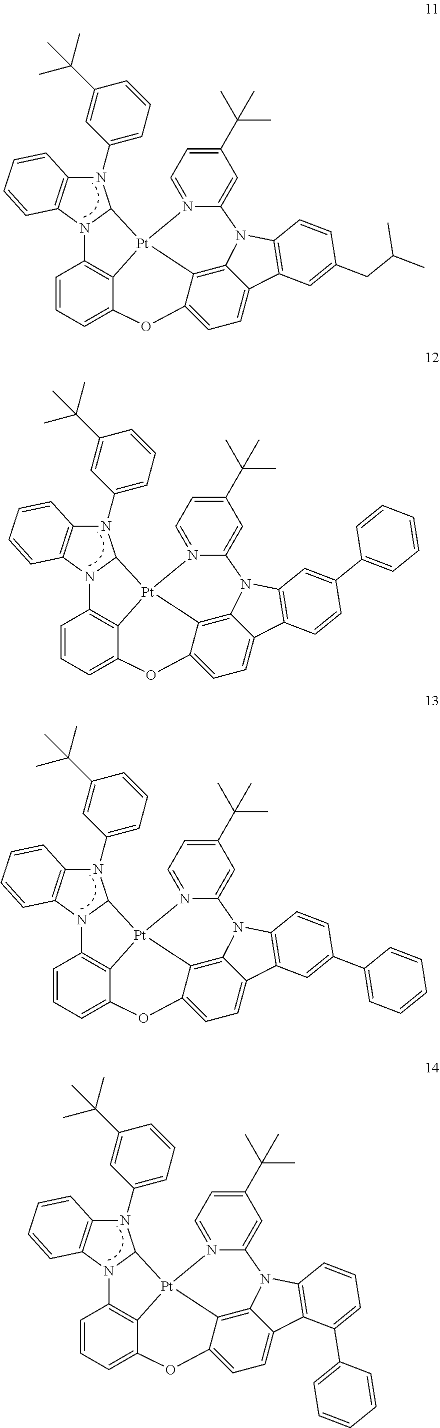

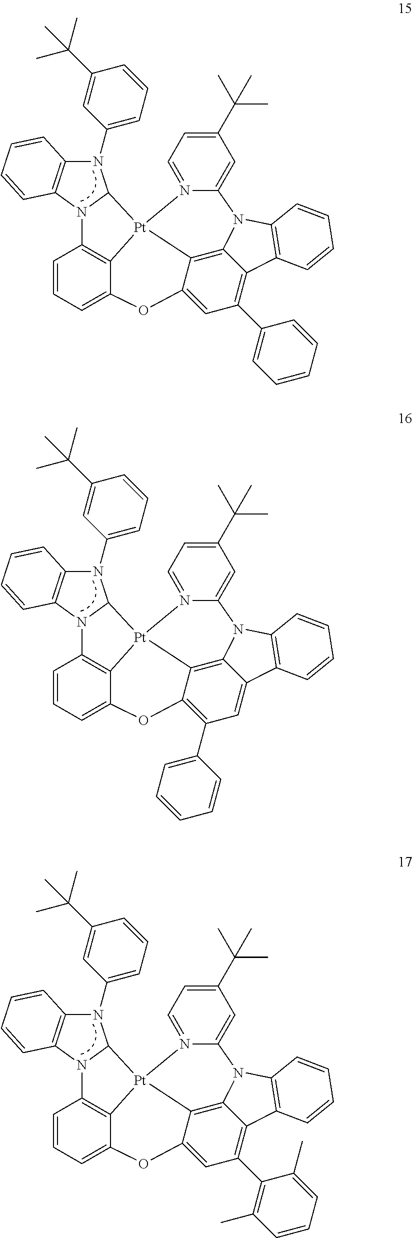

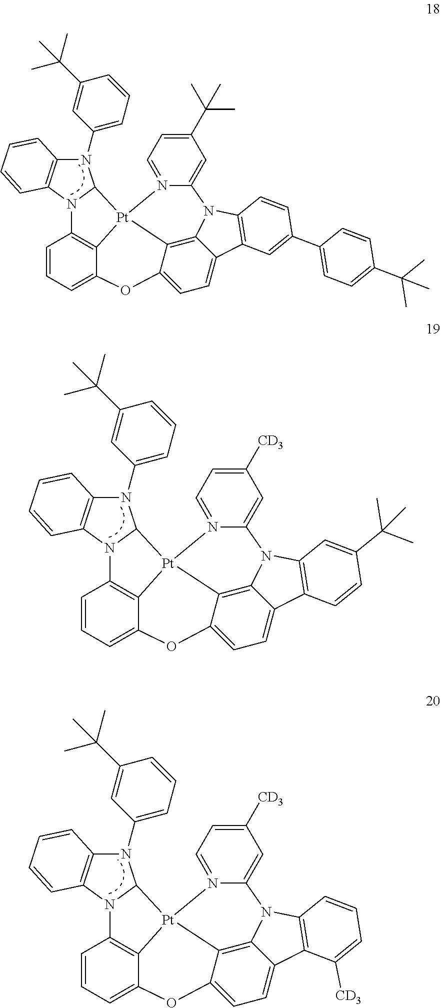

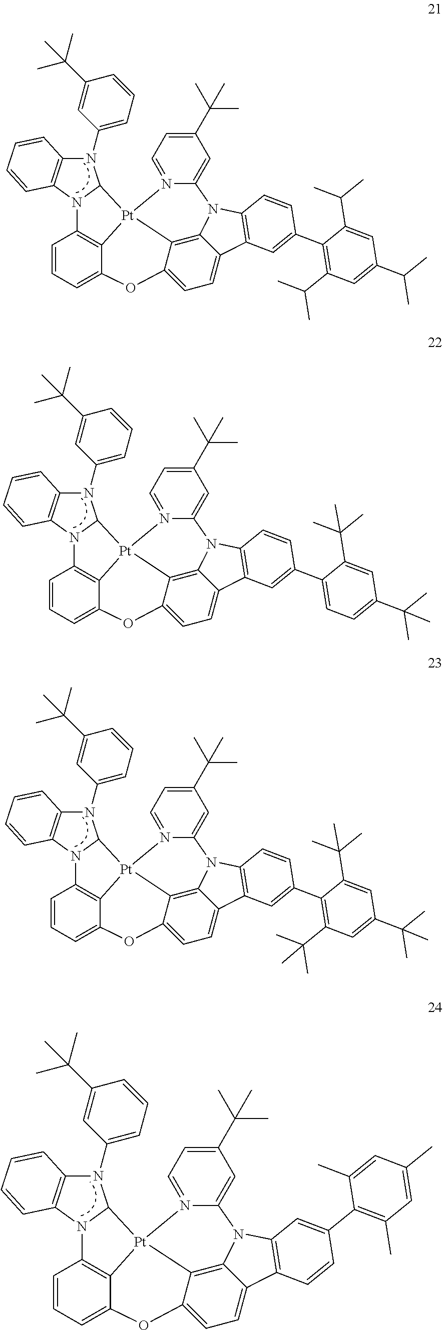

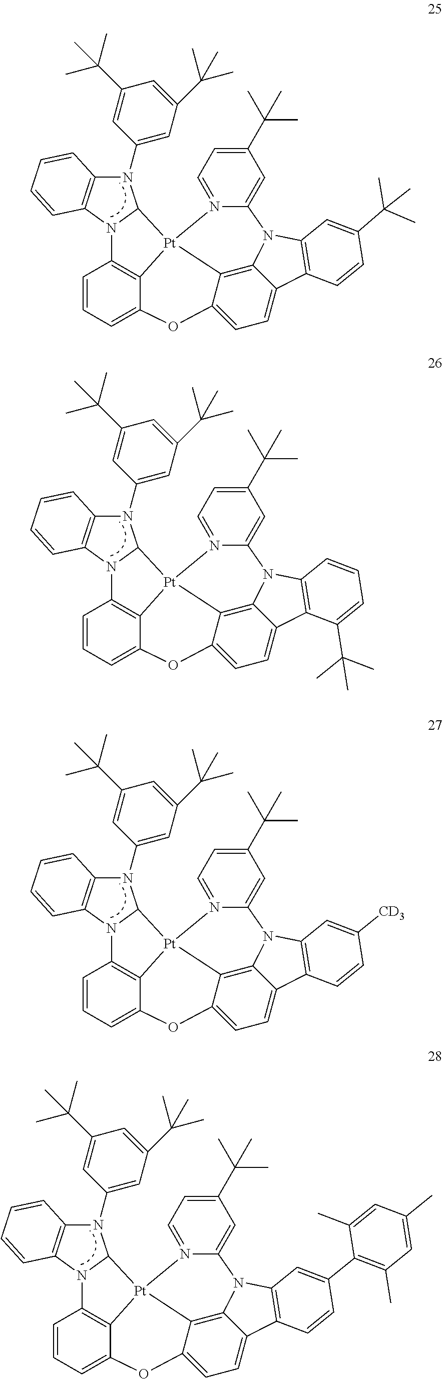

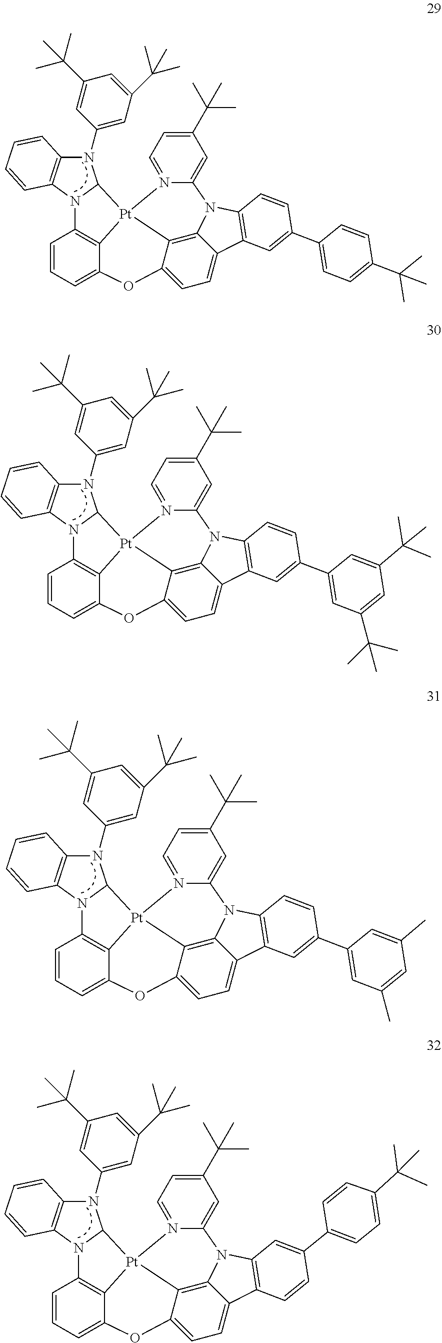

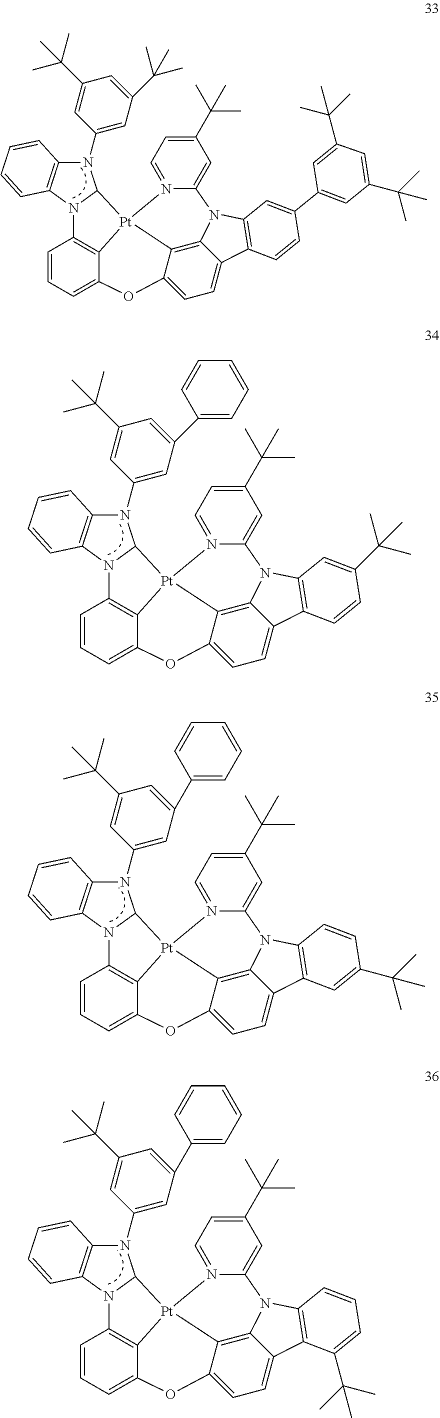

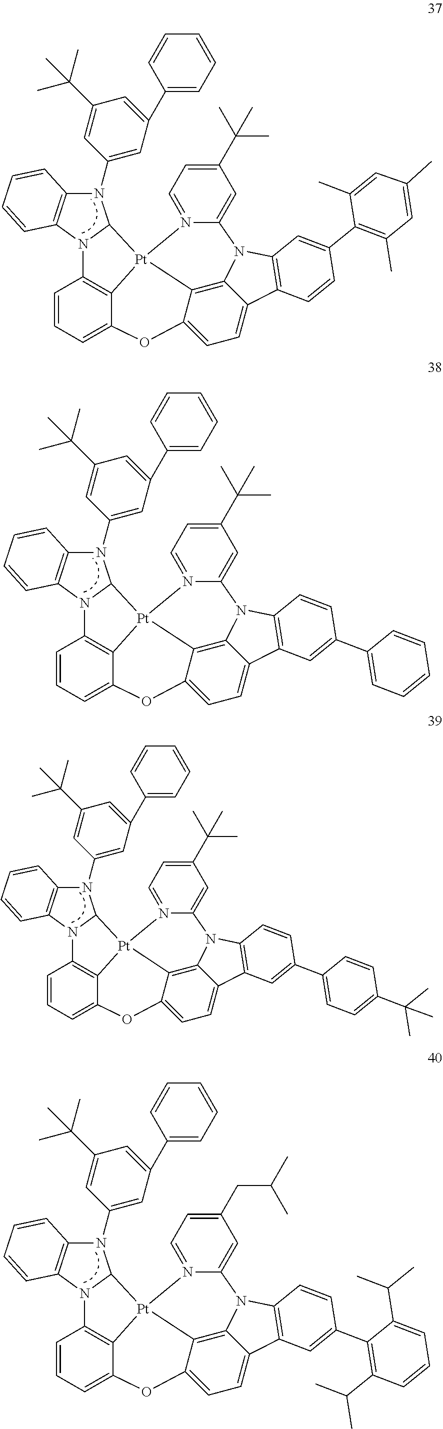

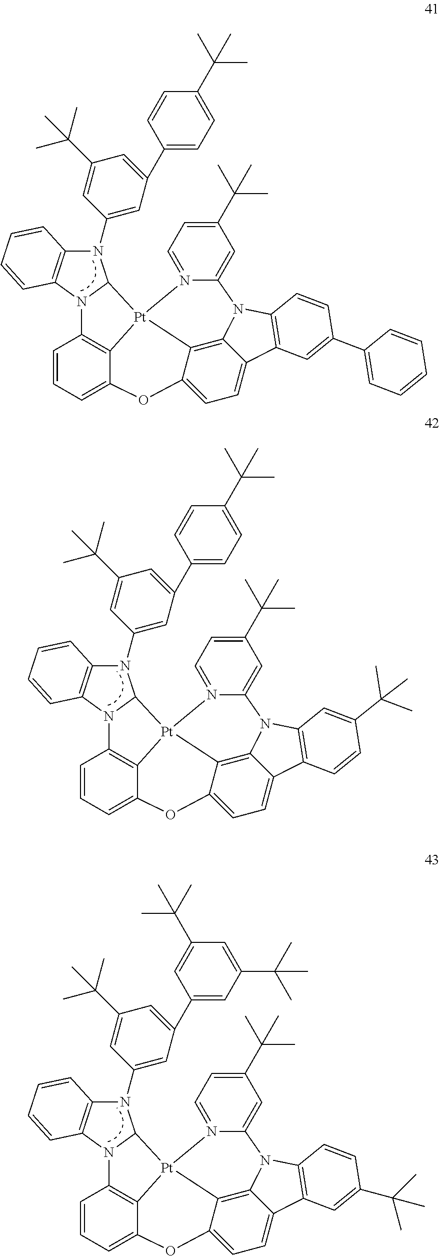

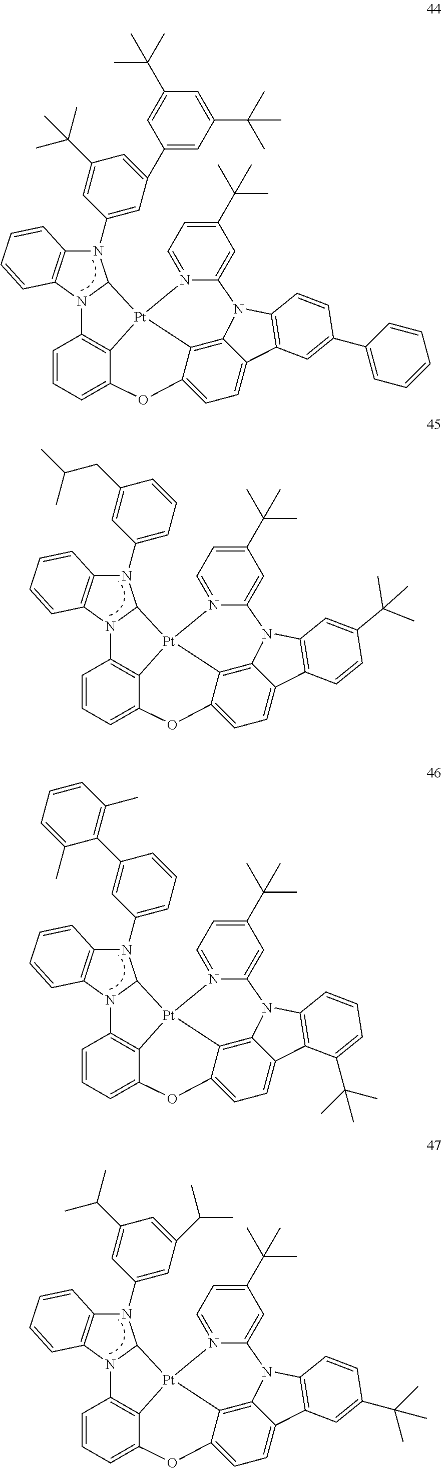

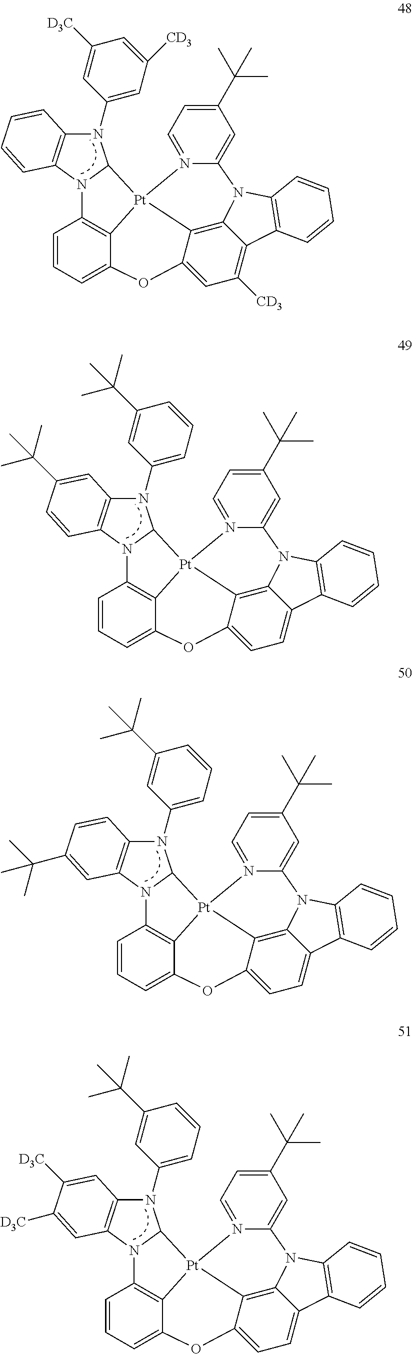

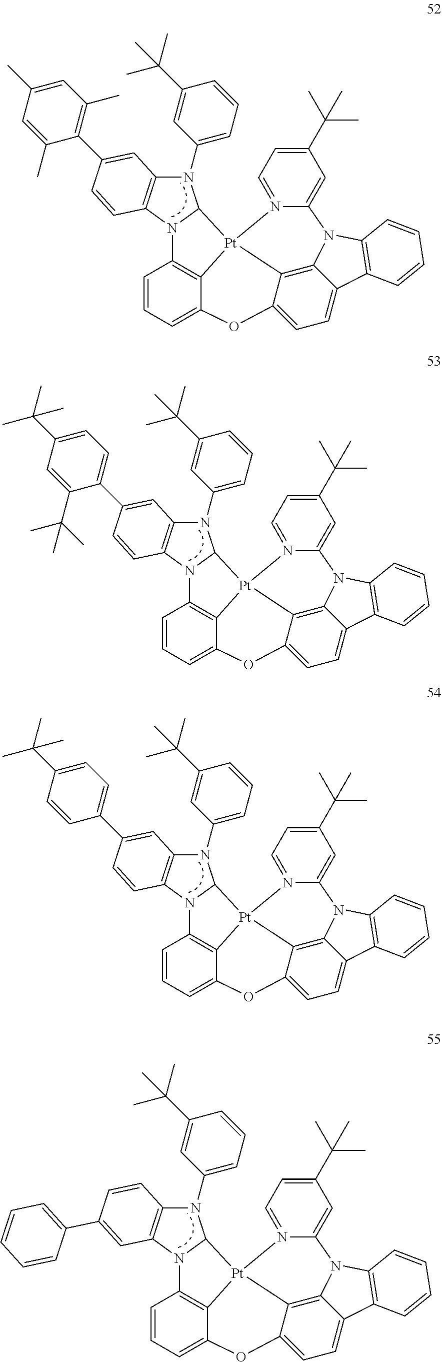

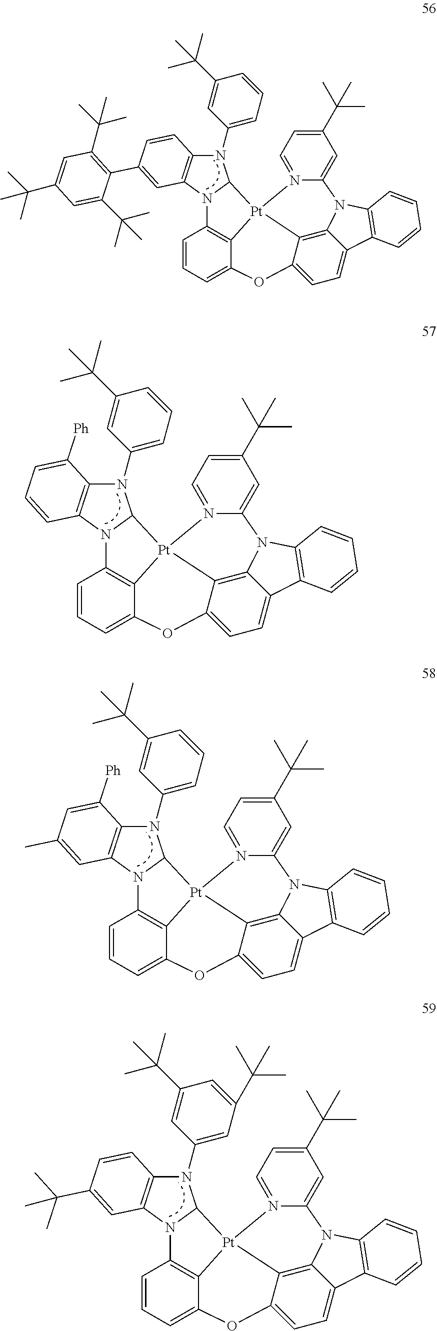

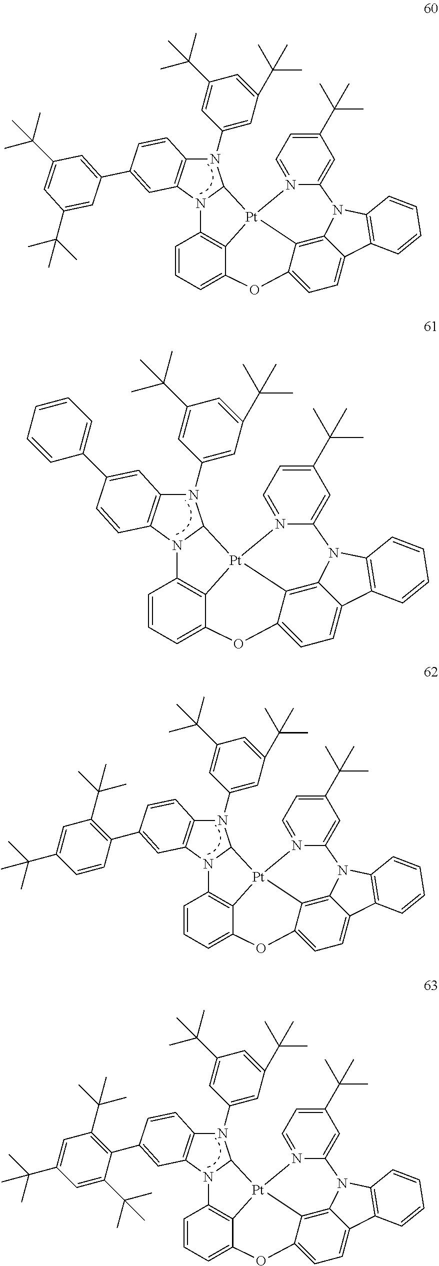

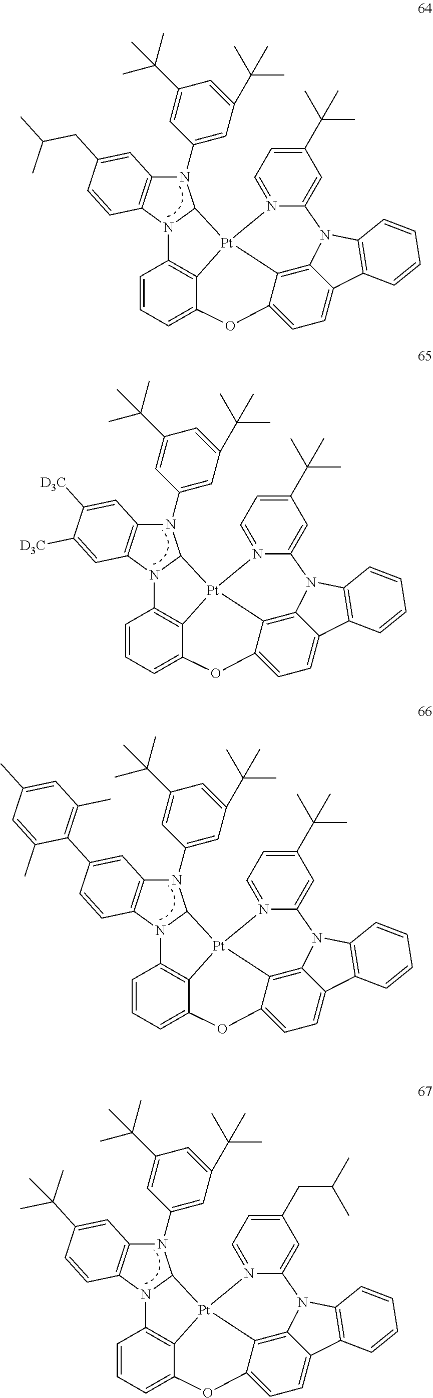

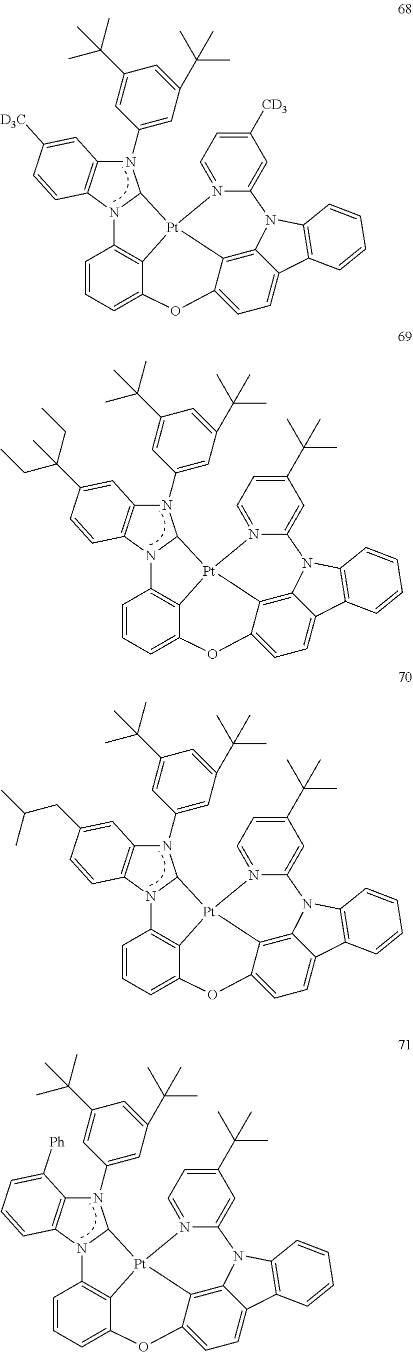

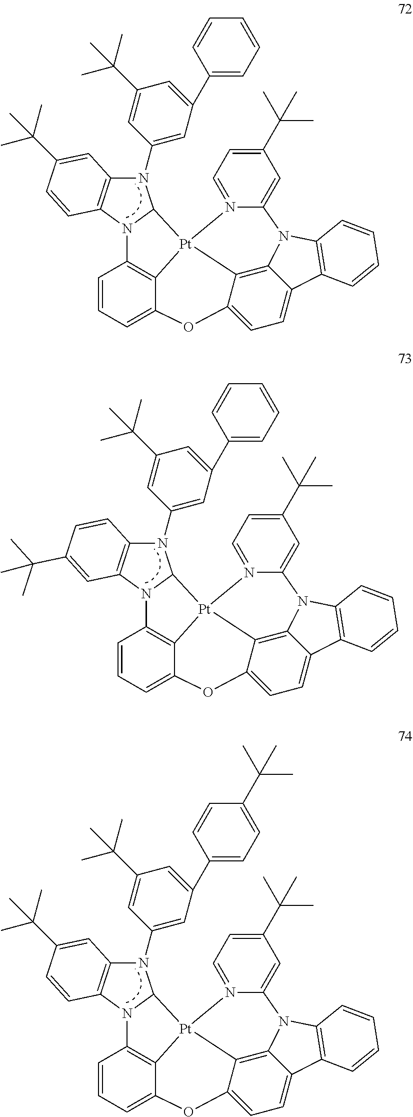

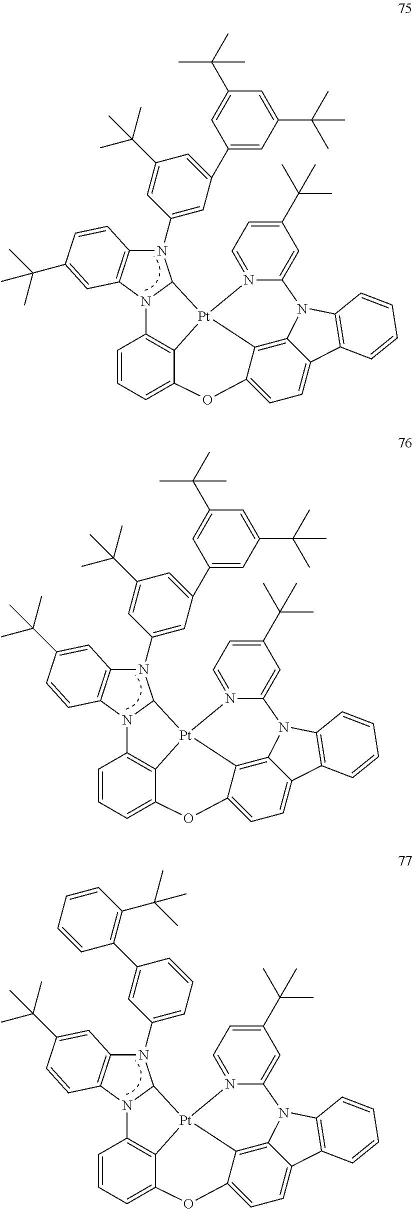

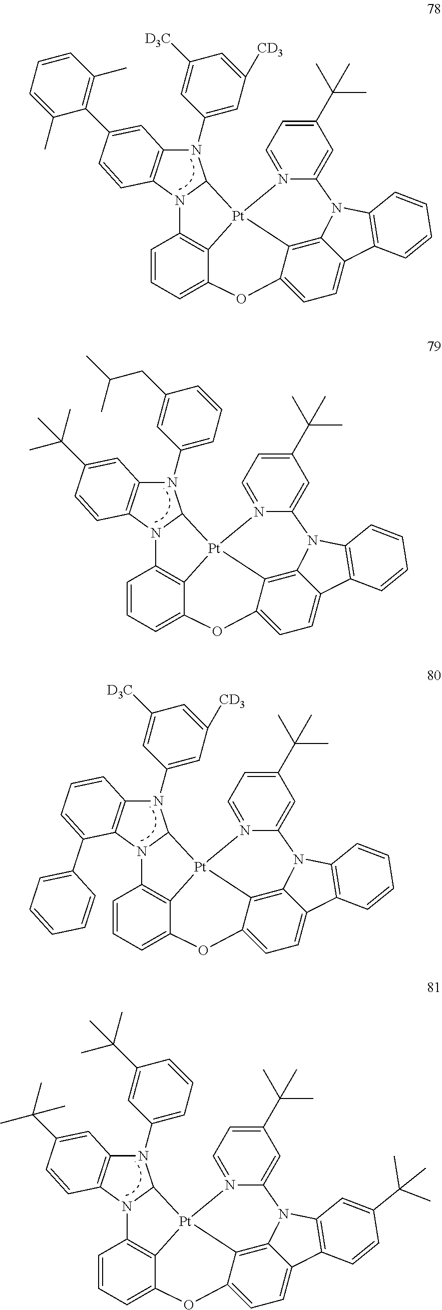

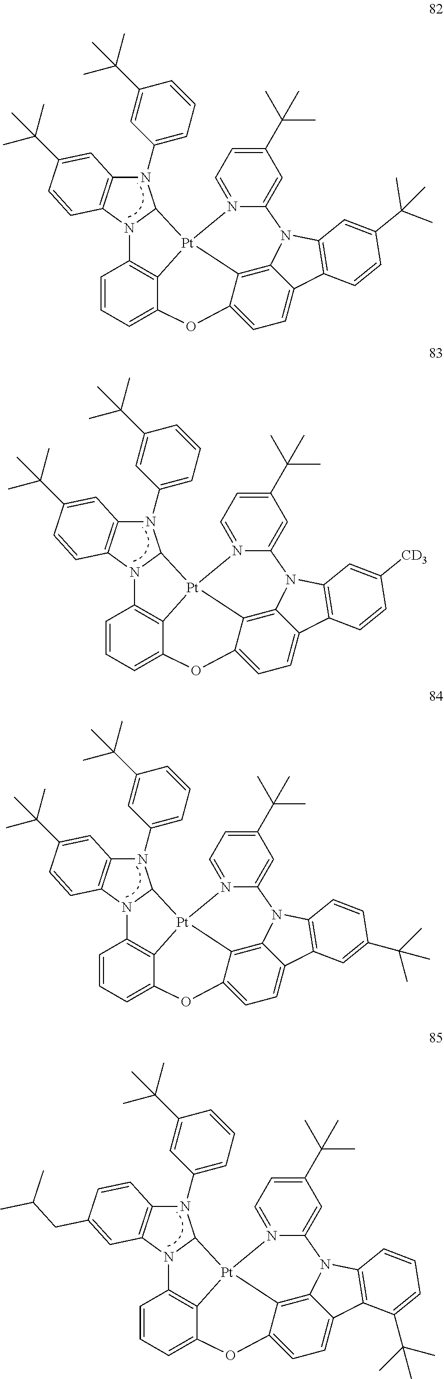

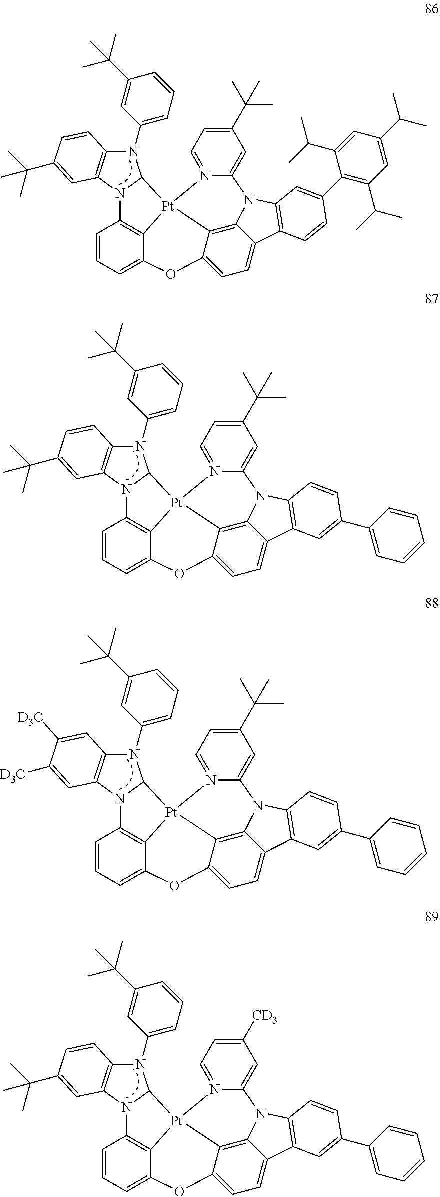

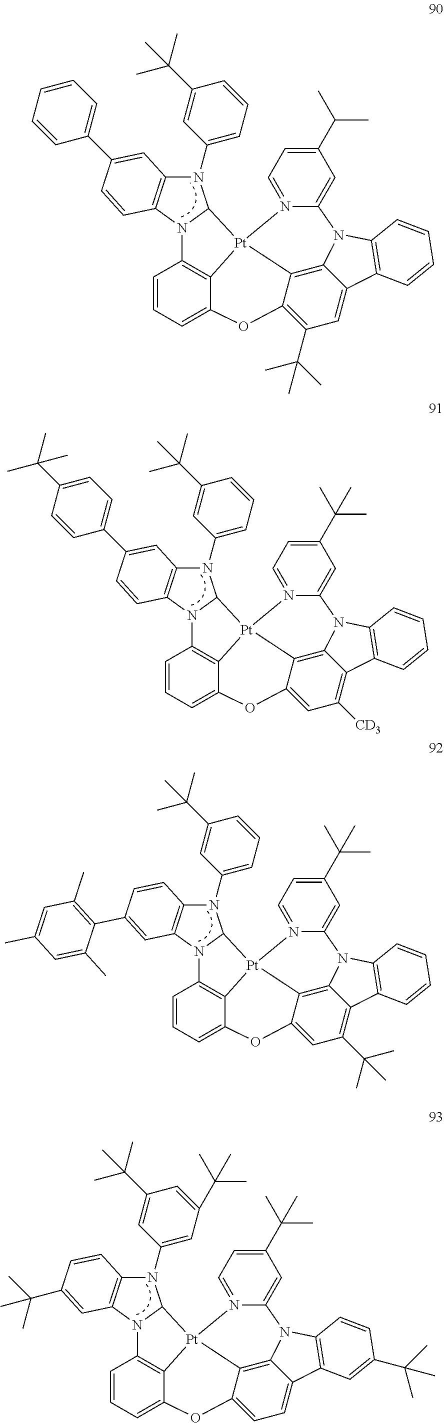

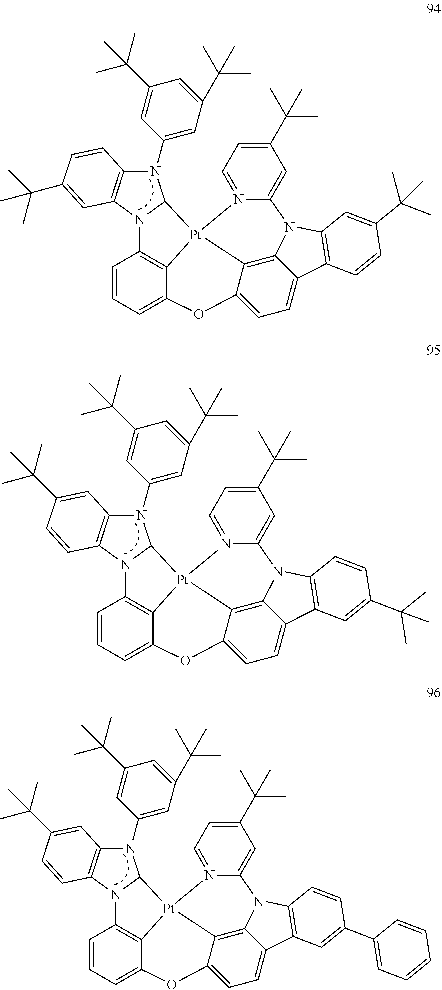

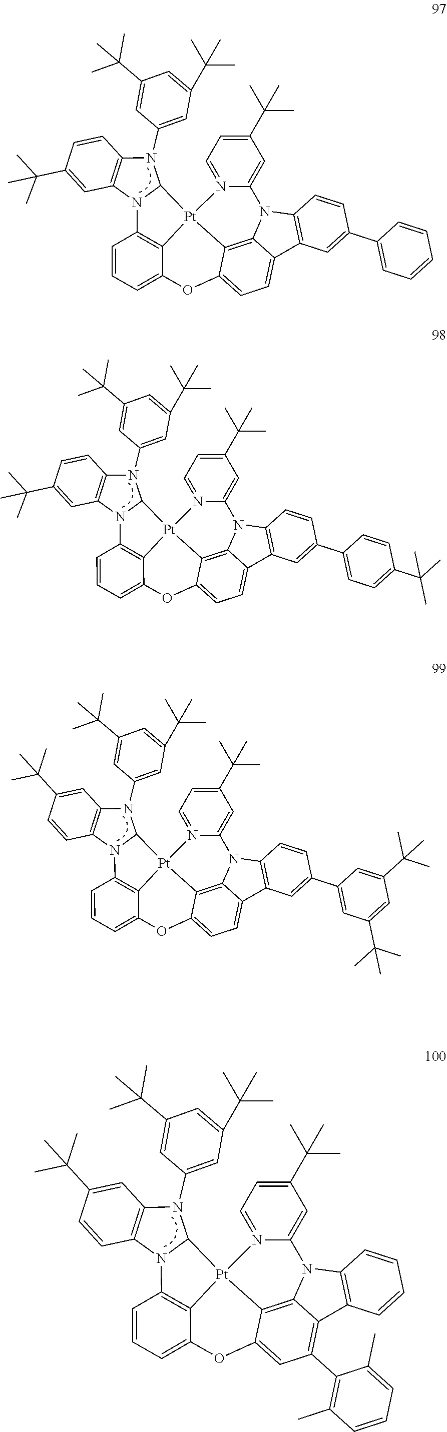

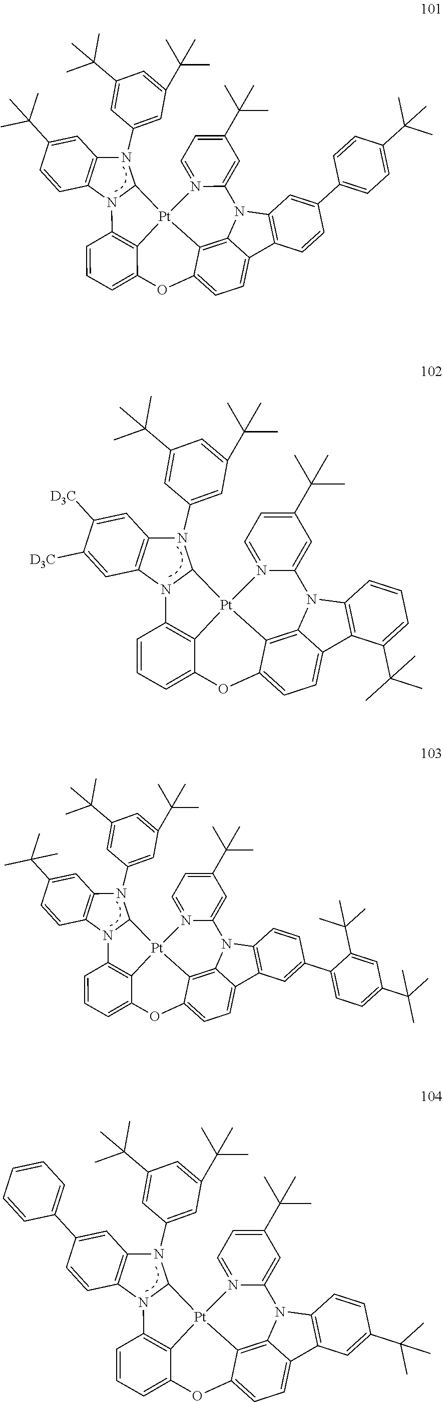

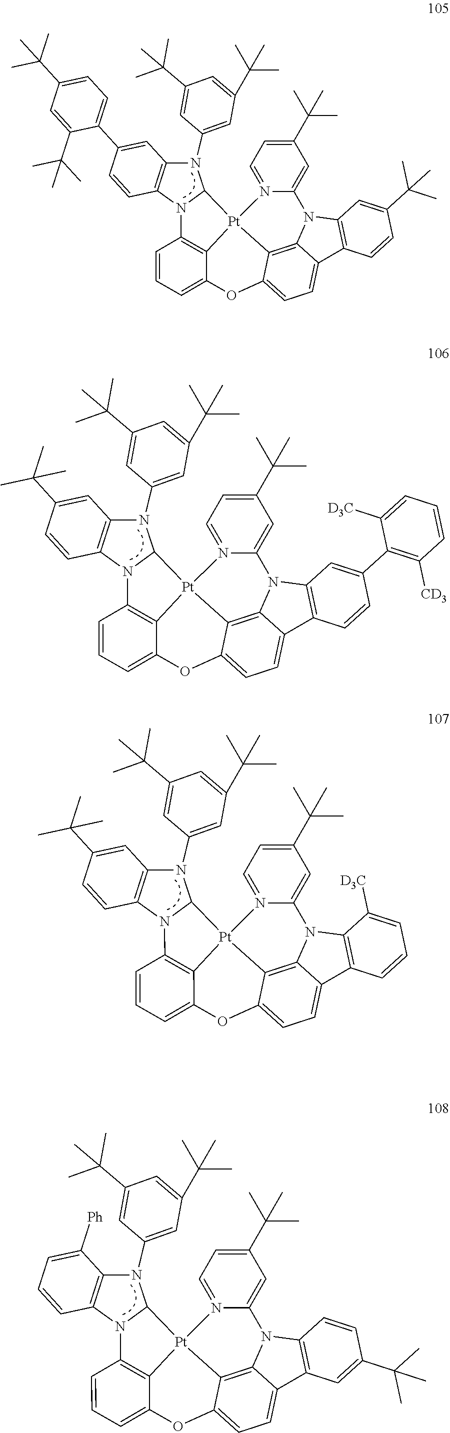

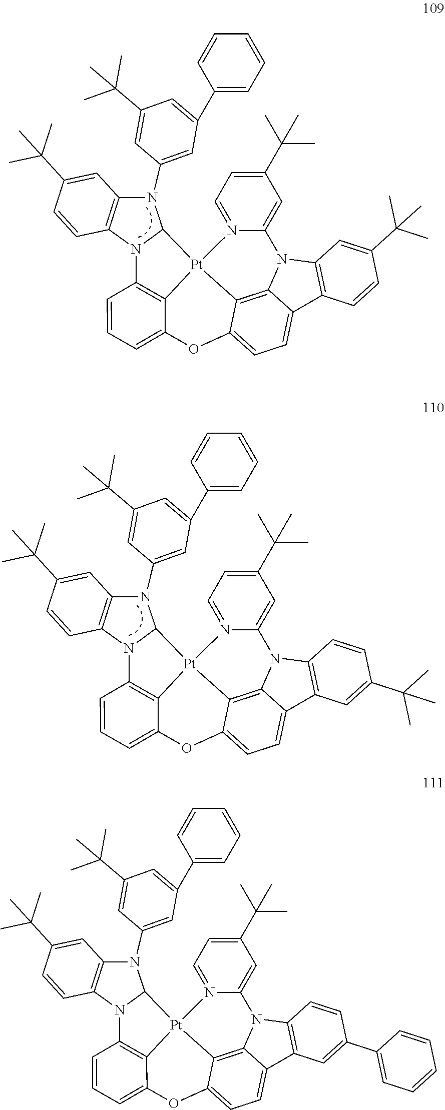

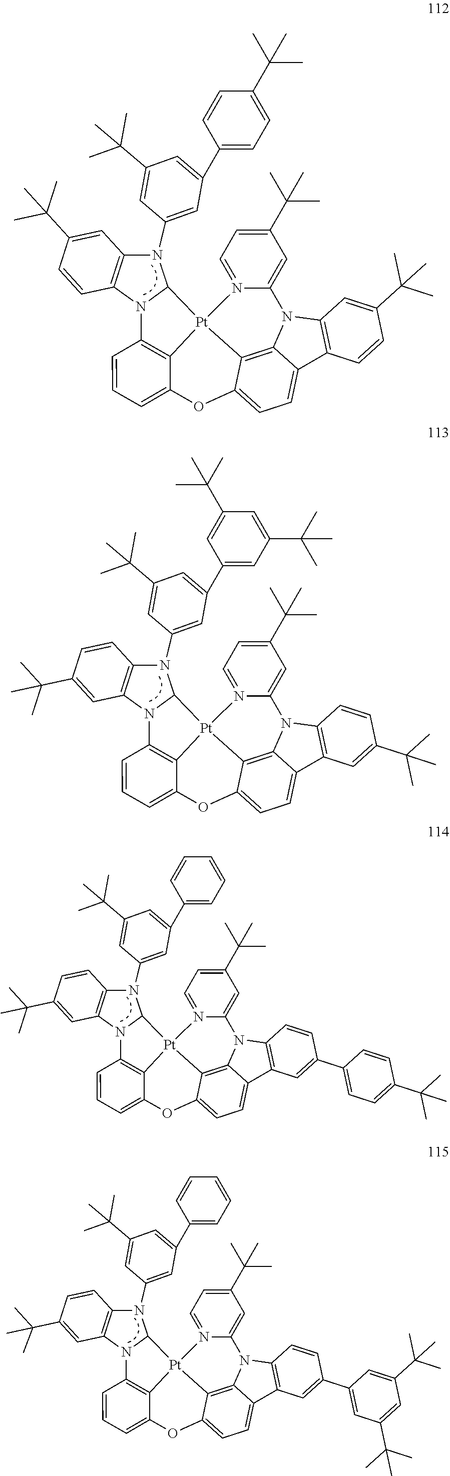

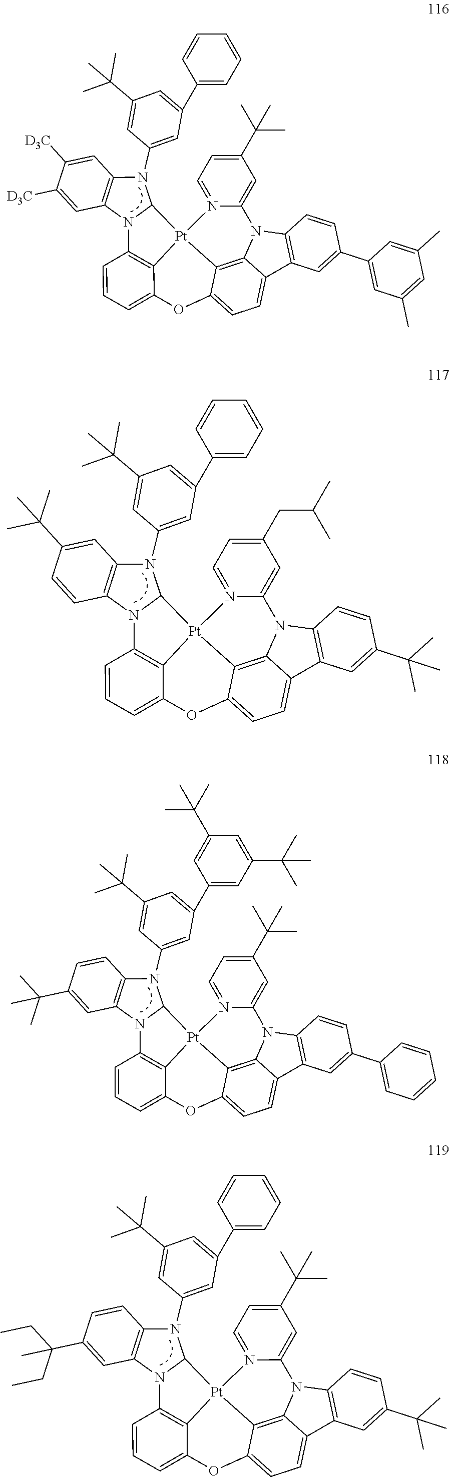

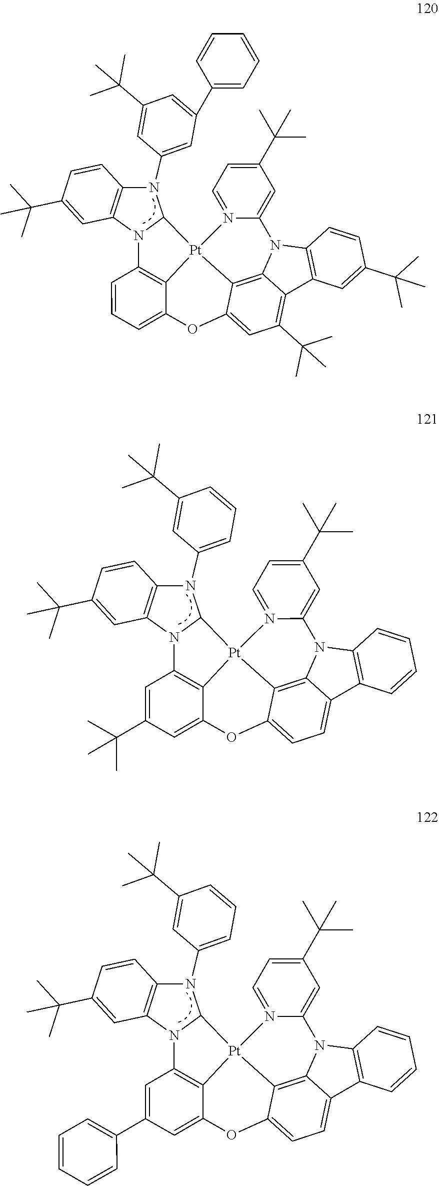

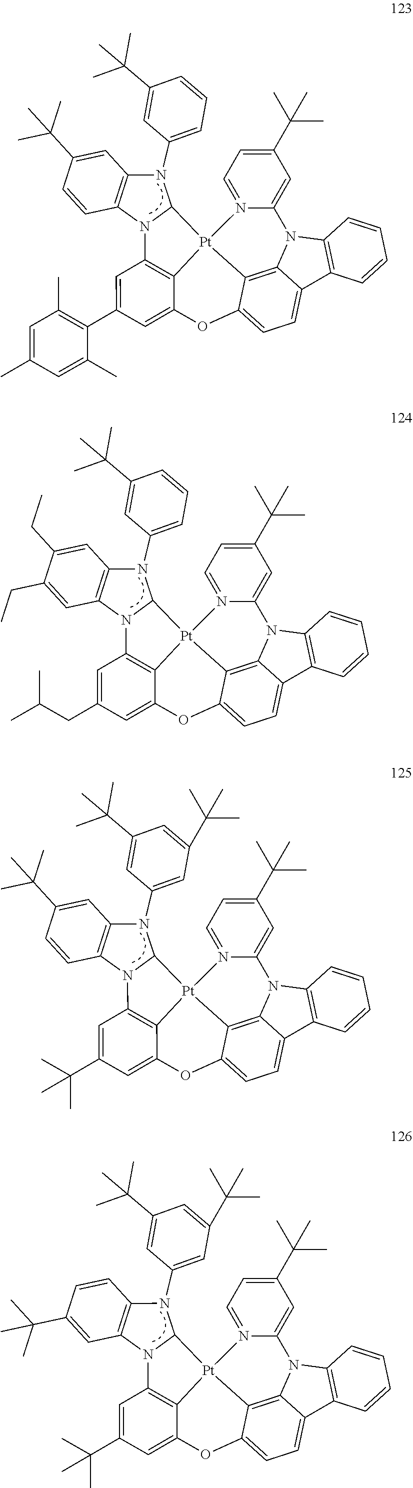

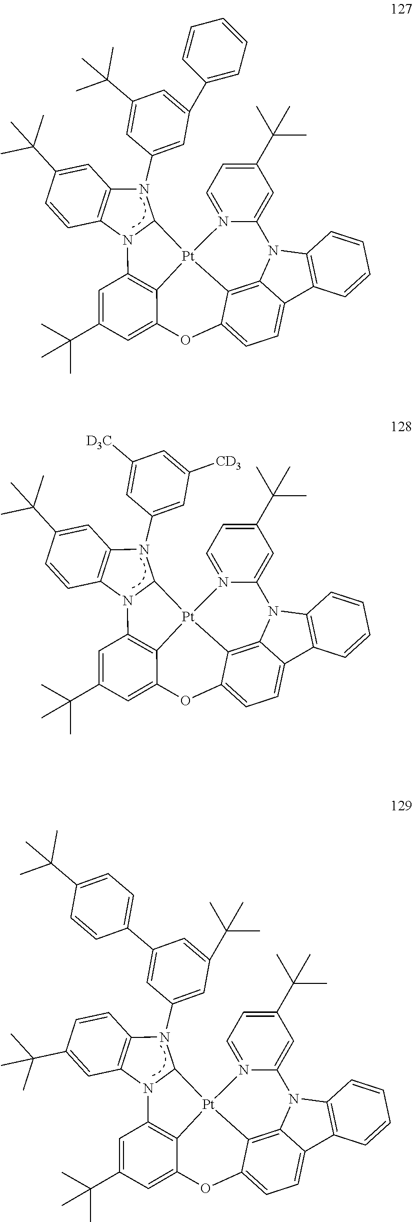

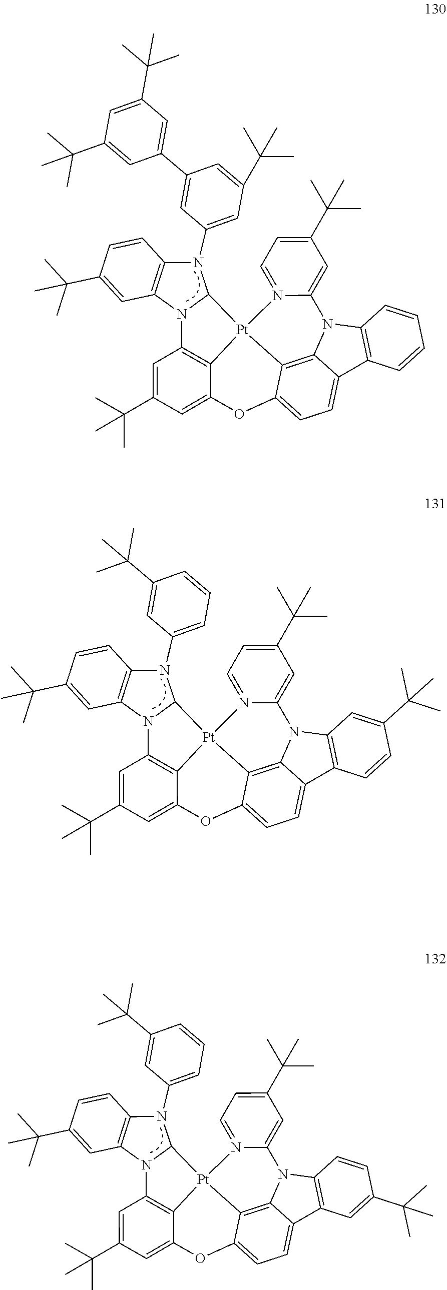

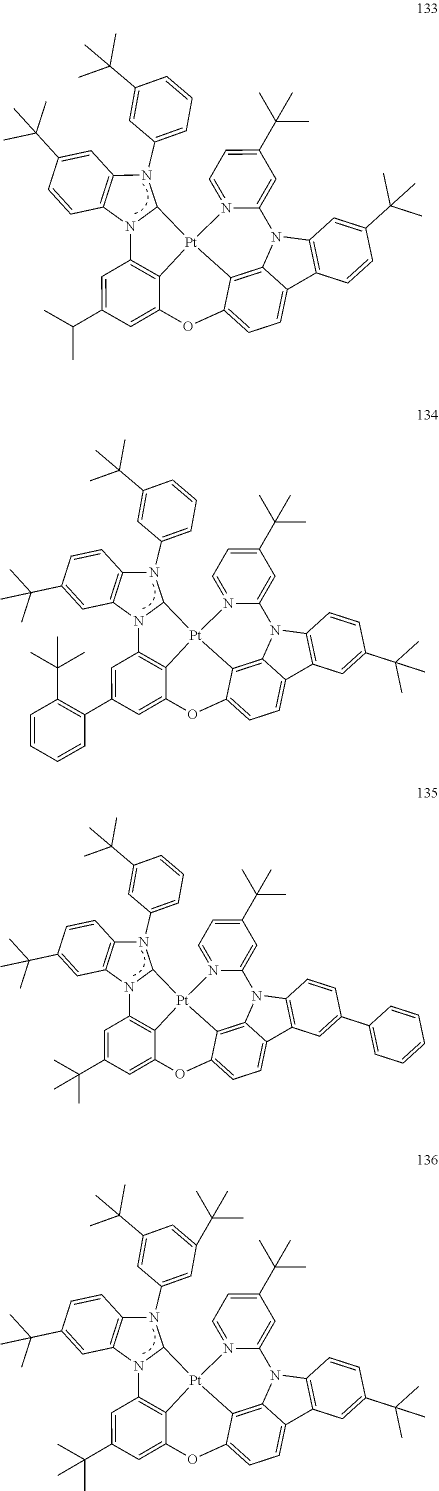

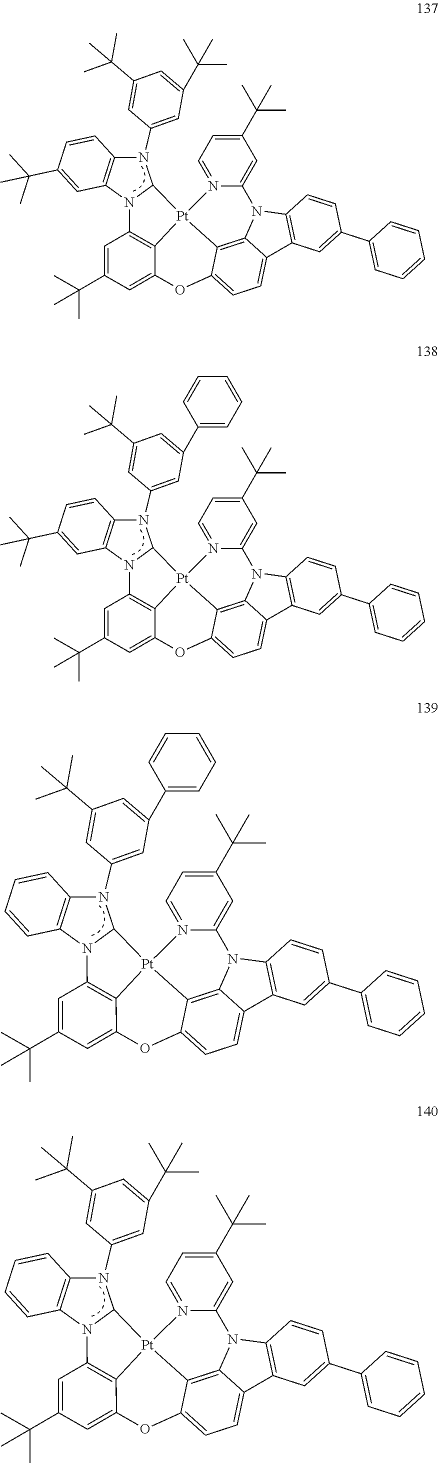

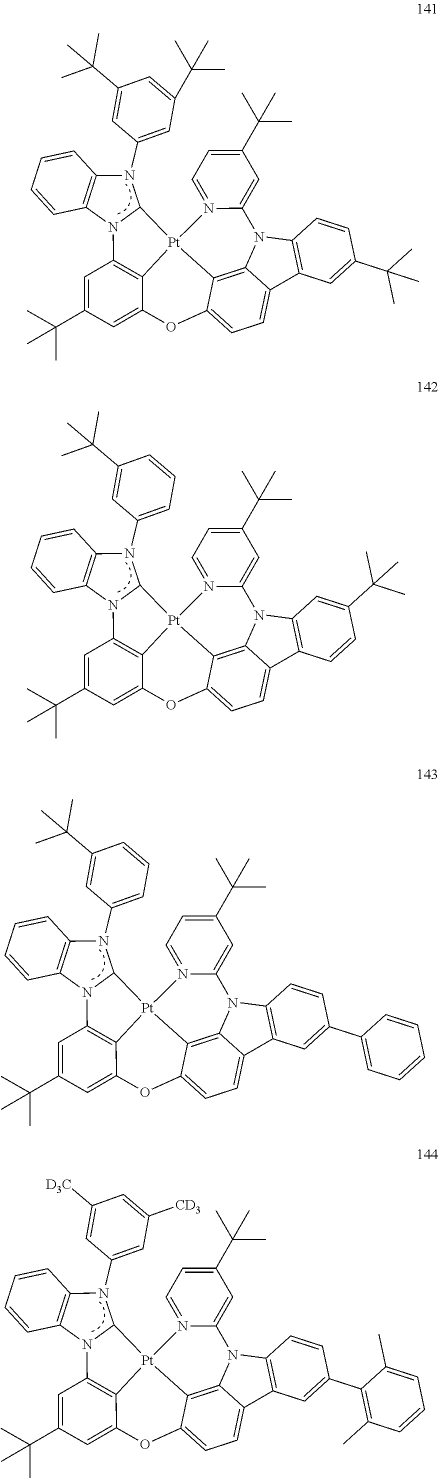

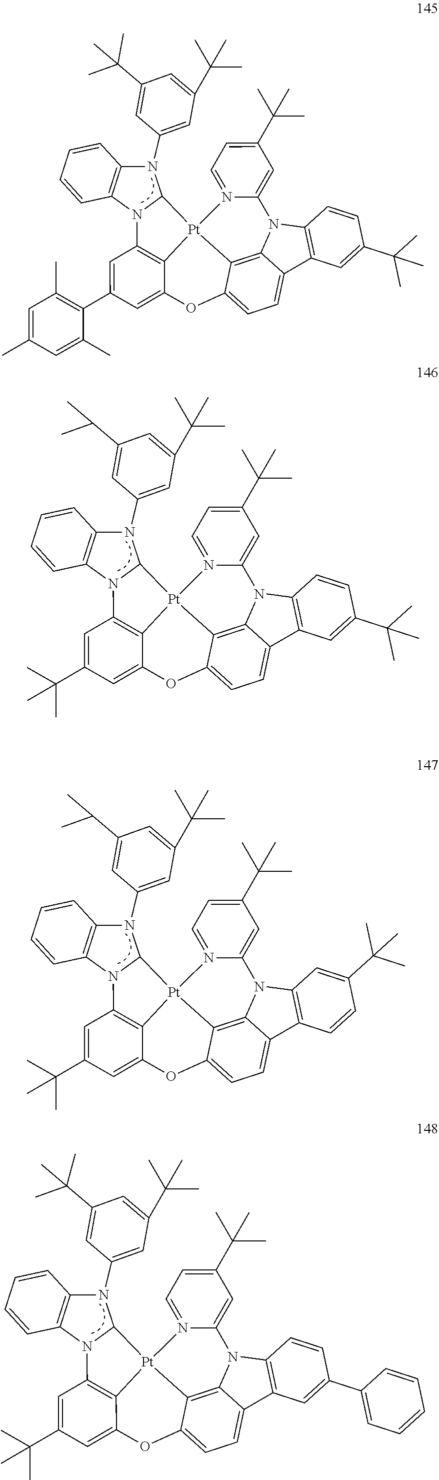

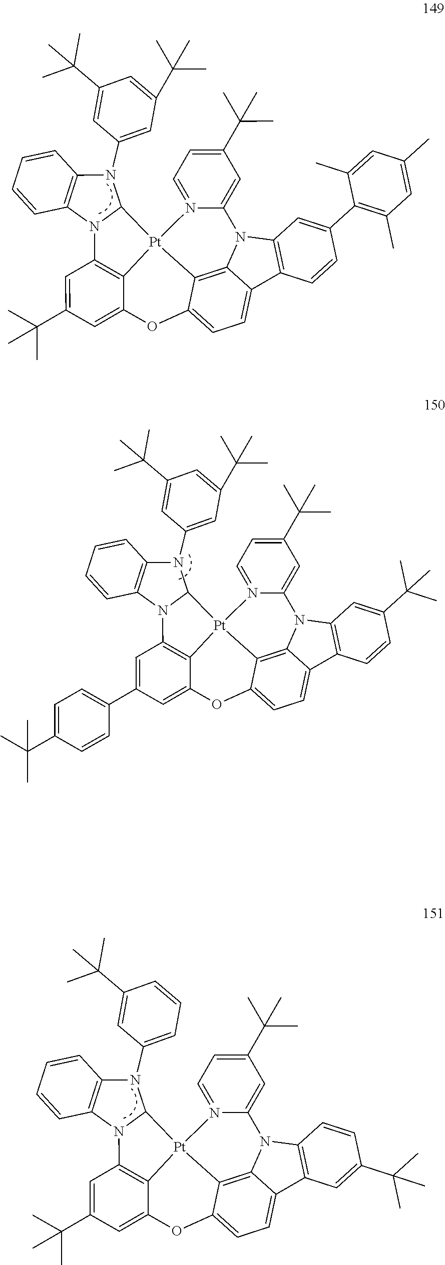

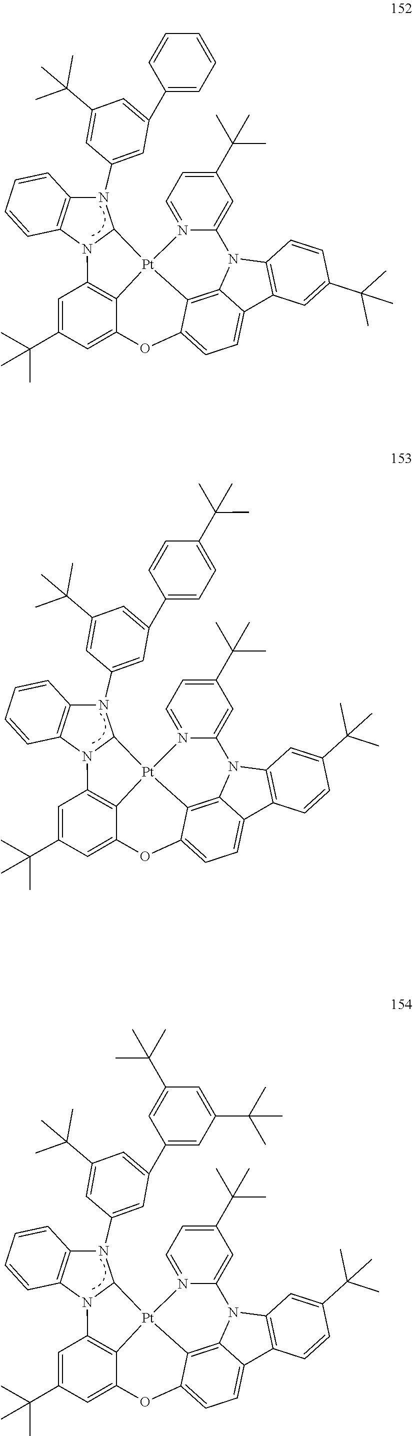

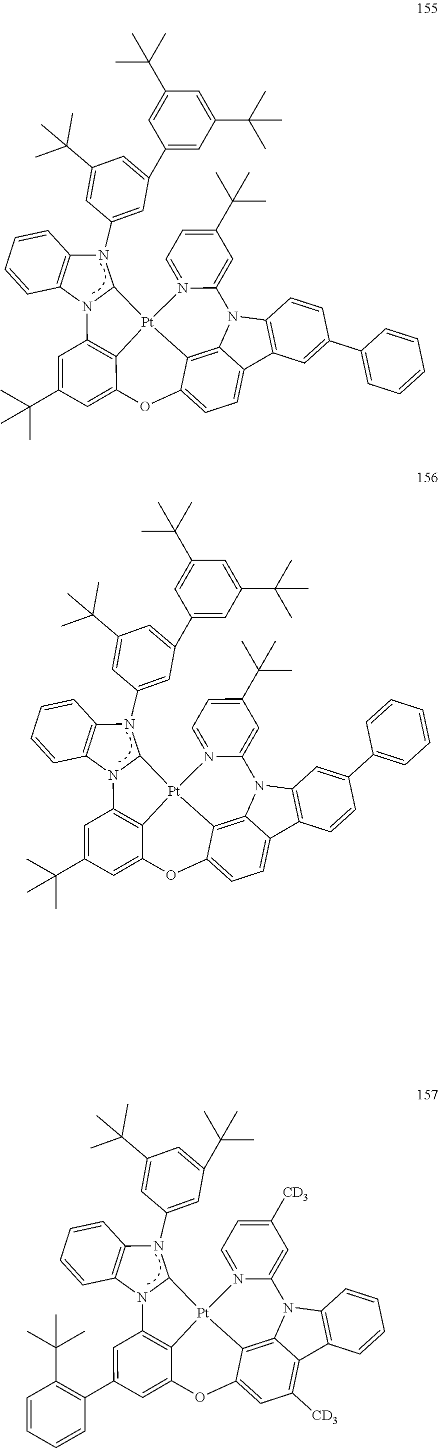

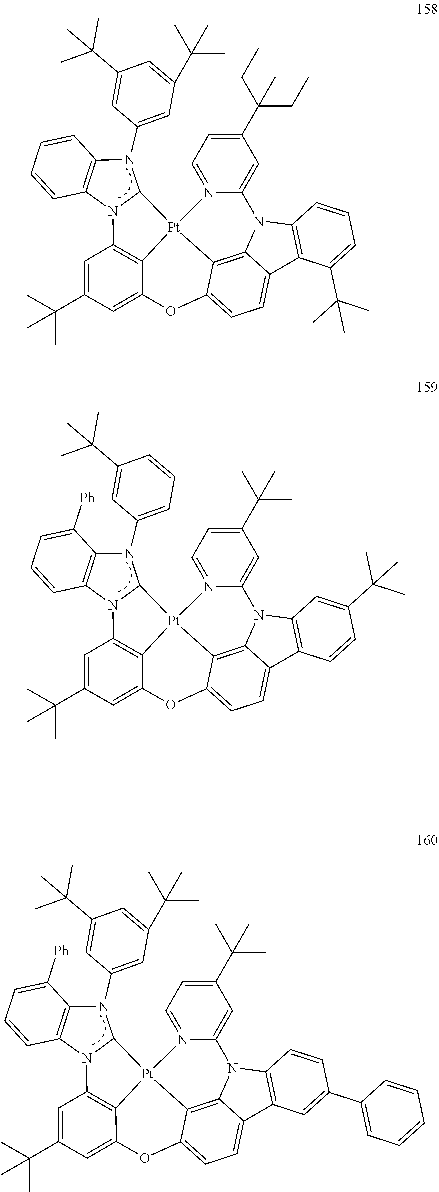

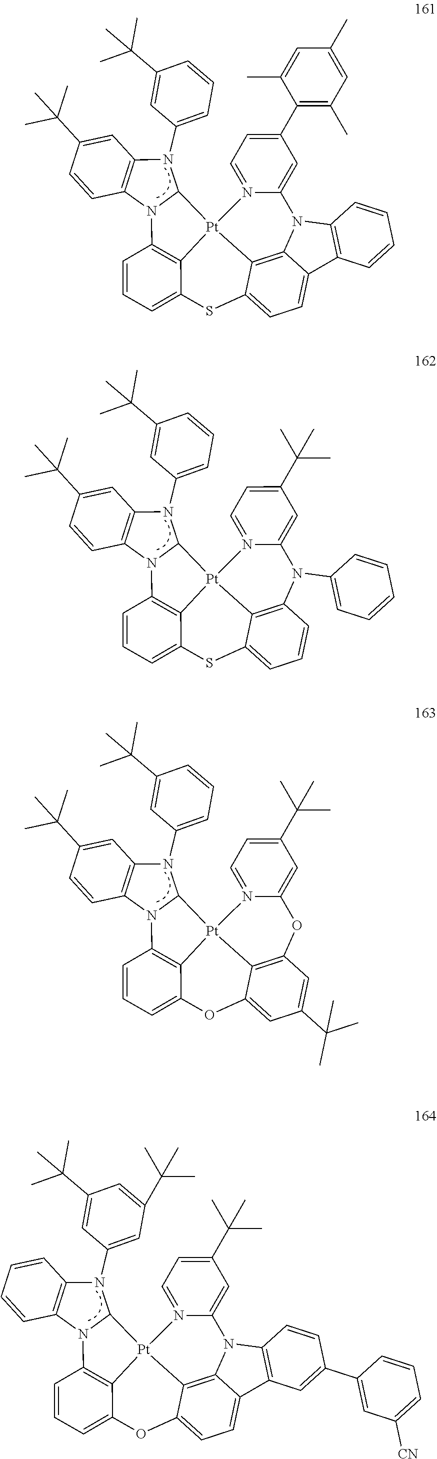

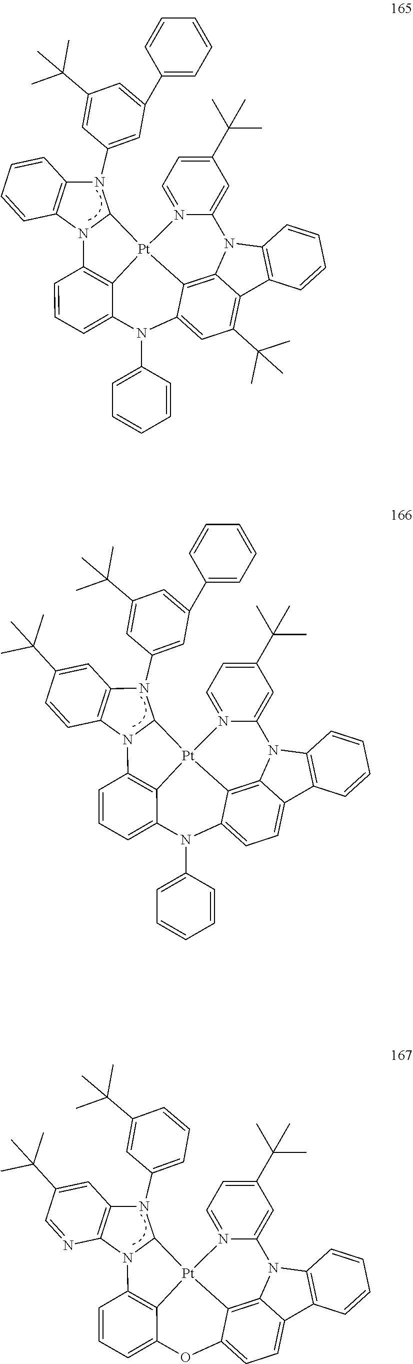

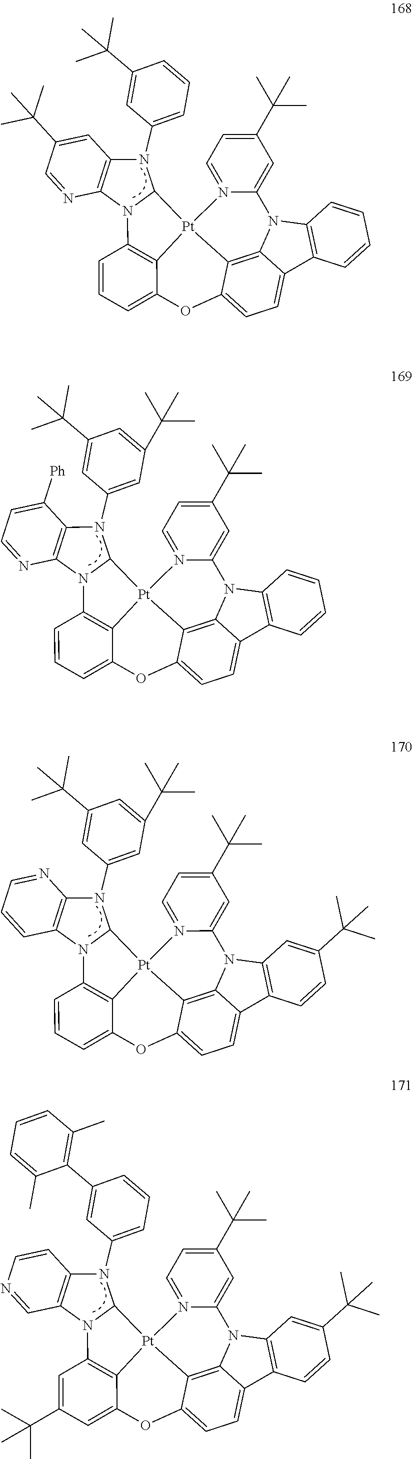

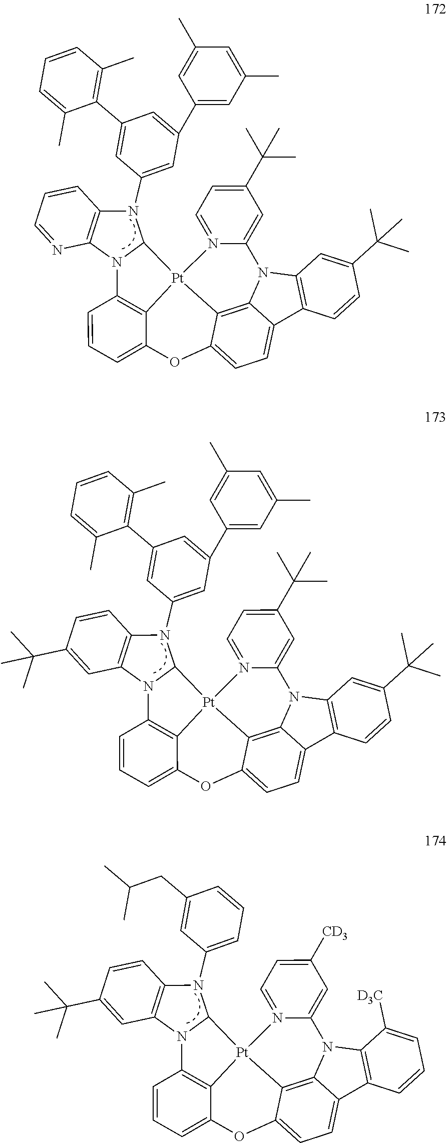

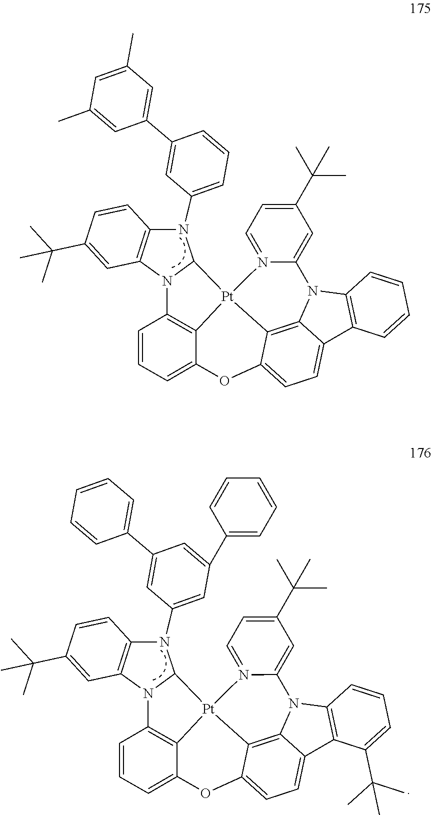

In one or more embodiments, the sensitizer may be represented by Formula 101 or Formula 102, and in this case, the sensitizer may be referred to as a delayed fluorescence sensitizer.

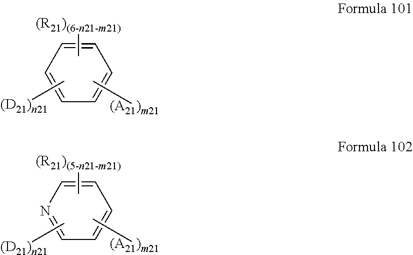

wherein, in Formulae 101 and 102,
A₂₁ may be an acceptor group,
D₂₁ may be a donor group,
m21 may be 1, 2, or 3, and n21 may be 1, 2, or 3,
the sum of n21 and m21 in Formula 101 may be less than or equal to 6, and the sum of n21 and m21 in Formula 102 may be less than or equal to 5,
R₂₁ may be hydrogen, deuterium, —F, —Cl, —Br, —I, SF₅, a hydroxyl group, a cyano group, a nitro group, an amidino group, a hydrazine group, a hydrazone group, a substituted or unsubstituted C₁-C₆₀ alkyl group, a substituted or unsubstituted C₂-C₆₀ alkenyl group, a substituted or unsubstituted C₂-C₆₀ alkynyl group, a substituted or unsubstituted C₁-C₆₀ alkoxy group, a substituted or unsubstituted C₃-C₁₀ cycloalkyl group, a substituted or unsubstituted C₁-C₁₀ heterocycloalkyl group, a substituted or unsubstituted C₃-C₁₀ cycloalkenyl group, a substituted or unsubstituted C₂-C₁₀ heterocycloalkenyl group, a substituted or unsubstituted C₆-C₆₀ aryl group, a substituted or unsubstituted C₇-C₆₀ alkylaryl group, a substituted or unsubstituted C₆-C₆₀ aryloxy group, a substituted or unsubstituted C₆-C₆₀ arylthio group, a substituted or unsubstituted C₁-C₆₀ heteroalkylaryl group, a substituted or unsubstituted C₂-C₆₀ heteroaryl alkyl group, a substituted or unsubstituted C₁-C₆₀ heteroaryloxy group, a substituted or unsubstituted C₁-C₆₀ heteroarylthio group, a substituted or unsubstituted monovalent non-aromatic condensed polycyclic group, a substituted or unsubstituted monovalent non-aromatic condensed heteropolycyclic group, —Si(Q₁)(Q₂)(Q₃), —Ge(Q₁)(Q₂)(Q₃), —B(Q₁)(Q₂), —N(Q₁)(Q₂), —P(Q₁)(Q₂), —C(═O)(Q₁), —S(═O)(Q₁), —S(═O)₂(Q₁), —P(═O)(Q₁)(Q₂), or —P(═S)(Q₁)(Q₂), wherein a plurality of R₂₁ may optionally be linked to each other to form a substituted or unsubstituted C₅-C₃₀ carbocyclic group or a substituted or unsubstituted C₁-C₃₀ heterocyclic group, and
Q₁ to Q₃ may each independently be hydrogen, deuterium, —F, —Cl, —Br, —I, a hydroxyl group, a cyano group, a nitro group, an amidino group, a hydrazine group, a hydrazone group, a C₁-C₆₀ alkyl group, a C₂-C₆₀ alkenyl group, a C₂-C₆₀ alkynyl group, a C₁-C₆₀ alkoxy group, a C₃-C₁₀ cycloalkyl group, a C₁-C₁₀ heterocycloalkyl group, a C₃-C₁₀ cycloalkenyl group, a C₂-C₁₀ heterocycloalkenyl group, a C₆-C₆₀ aryl group, a C₇-C₆₀ alkylaryl group, a C₆-C₆₀ aryloxy group, a C₆-C₆₀ arylthio group, a C₁-C₆₀ heteroaryl group, a C₂-C₆₀ heteroalkylaryl group, a C₁-C₆₀ heteroaryloxy group, a C₁-C₆₀ heteroarylthio group, a monovalent aromatic condensed polycyclic group, a monovalent aromatic condensed heteropolycyclic group, a monovalent non-aromatic condensed polycyclic group, a monovalent non-aromatic condensed heteropolycyclic group, a C₁-C₆₀ alkyl group that is substituted with at least one deuterium, —F, a cyano group, a C₁-C₆₀ alkyl group, a C₆-C₆₀ aryl group, or a combination thereof, or a C₆-C₆₀ aryl group that is substituted with deuterium, —F, a cyano group, a C₁-C₆₀ alkyl group, a C₆-C₆₀ aryl group, or a combination thereof.

For example, A₂₁ in Formulae 101 and 102 may be a substituted or unsubstituted π electron-deficient nitrogen-free cyclic group.

In an embodiment, the π electron-deficient nitrogen-free cyclic group may be a benzene group, a heptalene group, an indene group, a naphthalene group, an azulene group, an indacene group, an acenaphthylene group, a fluorene group, a spiro-bifluorene group, a benzofluorene group, a dibenzofluorene group, a phenalene group, a phenanthrene group, an anthracene group, a fluoranthene group, a triphenylene group, a pyrene group, a chrysene group, a naphthacene group, a picene group, a perylene group, a pentacene group, a hexacene group, a pentacene group, a rubicene group, a coragen group, an ovalene group, a pyrrole group, an isoindole group, an indole group, a furan group, a thiophene group, a benzofuran group, a benzothiophene group, a benzocarbazole group, a dibenzocarbazole group, a dibenzofuran group, a dibenzothiophene group, a dibenzothiophene sulfone group, a carbazole group, a dibenzosilole group, an indenocarbazole group, an indolocarbazole group, a benzofurocarbazole group, a benzothienocarbazole group, a triindolobenzene group; or a condensed cyclic group of two or more π electron-deficient nitrogen-free cyclic groups, but embodiments of the present disclosure are not limited thereto.

For example, D₂₁ in Formulae 101 and 102 may be: —F, a cyano group, or a π-electron-deficient nitrogen-containing cyclic group;
a C₁-C₆₀ alkyl group, an π-electron deficient nitrogen-containing cyclic group, or an π electron-deficient nitrogen-free cyclic group, each substituted with at least one —F, a cyano group, or a combination thereof; or
an π-electron deficient nitrogen-containing cyclic group, each substituted with at least one deuterium, a C₁-C₆₀ alkyl group, an π-electron deficient nitrogen-containing cyclic group, an π electron-deficient nitrogen-free cyclic group, or a combination thereof.

In an embodiment, the π electron-deficient nitrogen-free cyclic group is the same as described above.

The term “π electron-deficient nitrogen-containing cyclic group” used herein refers to a cyclic group having at least one *—N=*′ moiety, and, for example, may be an imidazole group, a pyrazole group, a thiazole group, an isothiazole group, an oxazole group, an isoxazole group, a pyridine group, a pyrazine group, a pyridazine group, a pyrimidine group, an indazole group, a purine group, a quinoline group, an isoquinoline group, a benzoquinoline group, a phthalazine group, a naphthyridine group, a quinoxaline group, a quinazoline group, a cinnoline group, a phenanthridine group, an acridine group, a phenanthroline group, a phenazine group, a benzimidazole group, an isobenzothiazole group, a benzoxazole group, an isobenzoxazole group, a triazole group, a tetrazole group, an oxadiazole group, a triazine group, a thiadiazole group, an imidazopyridine group, an imidazopyrimidine group, an azacarbazole group, a benzimidazolobenzimidazole group; or a condensed cyclic group in which two or more π electron-deficient nitrogen-containing cyclic groups are condensed with each other.

In one or more embodiments, the sensitizer may be a compound of Groups XI to XV, but embodiments of the present disclosure are not limited thereto:

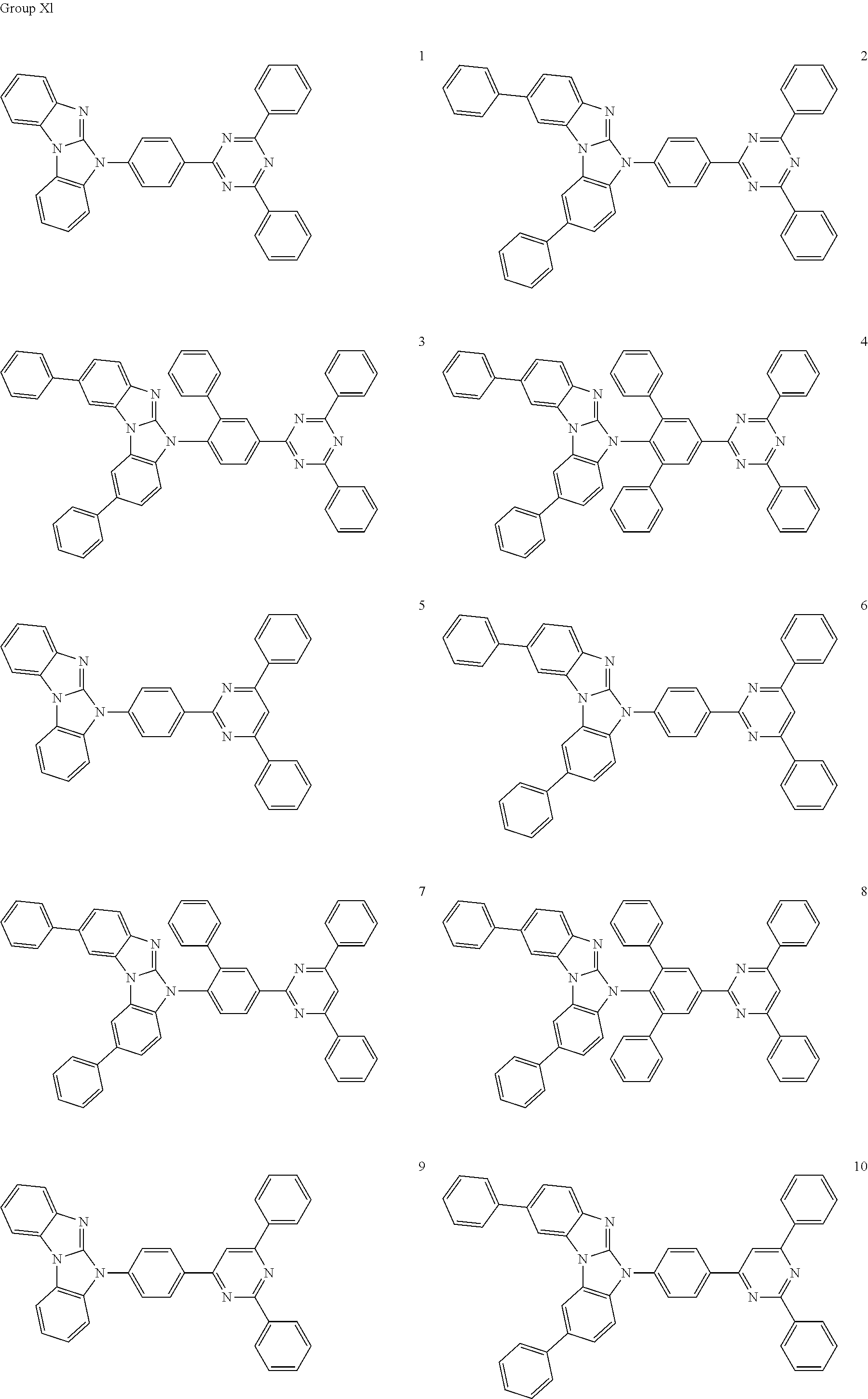

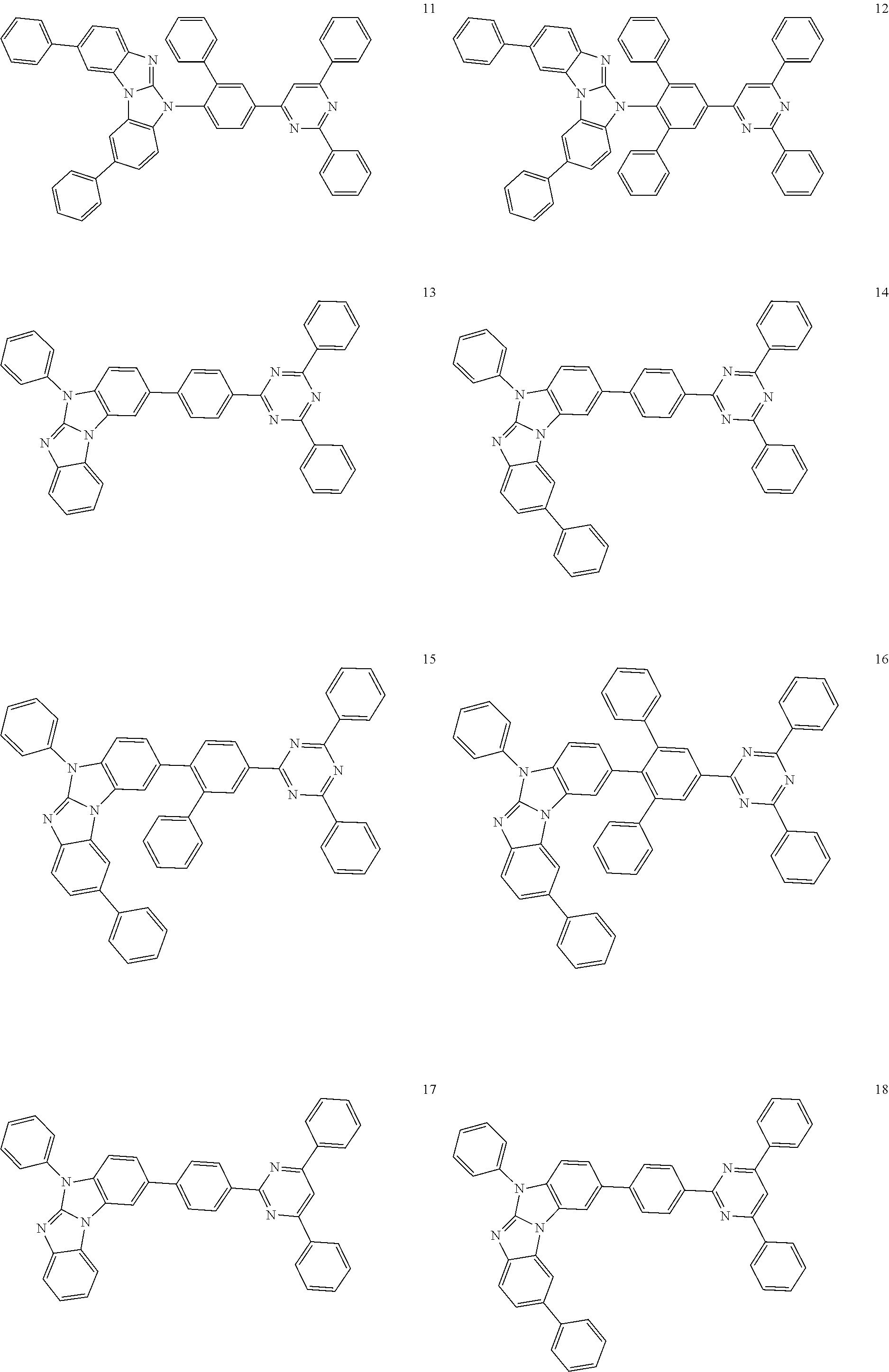

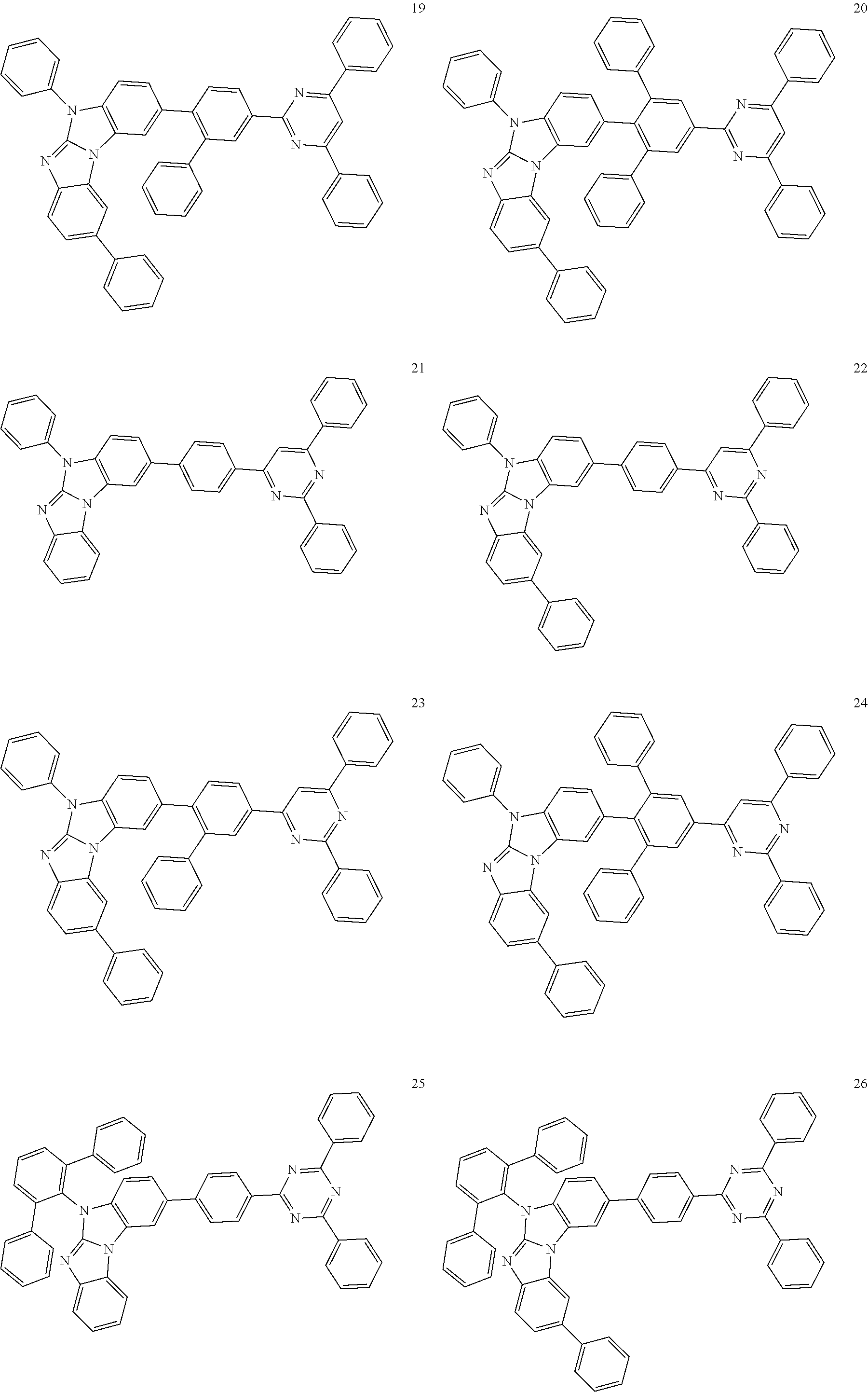

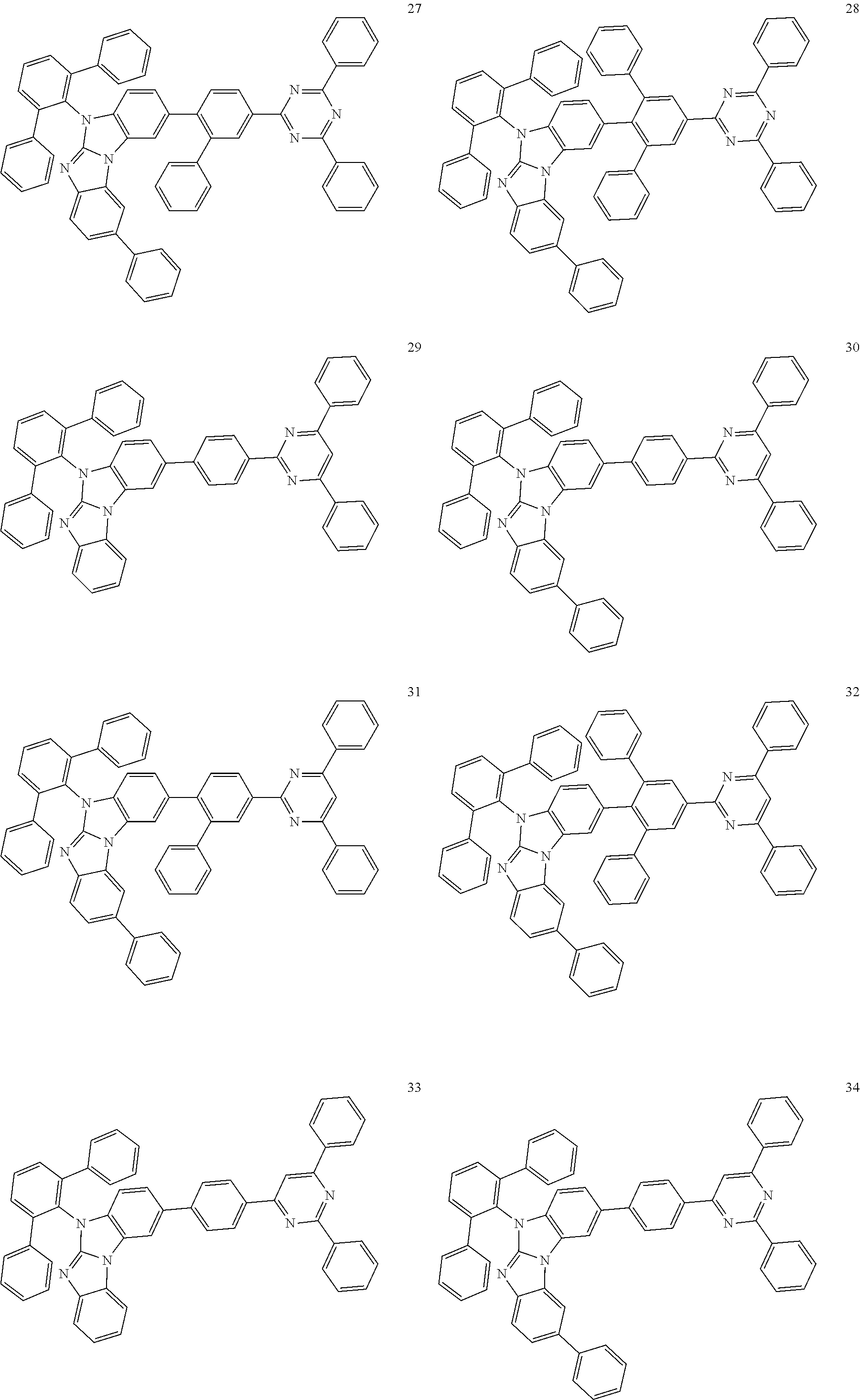

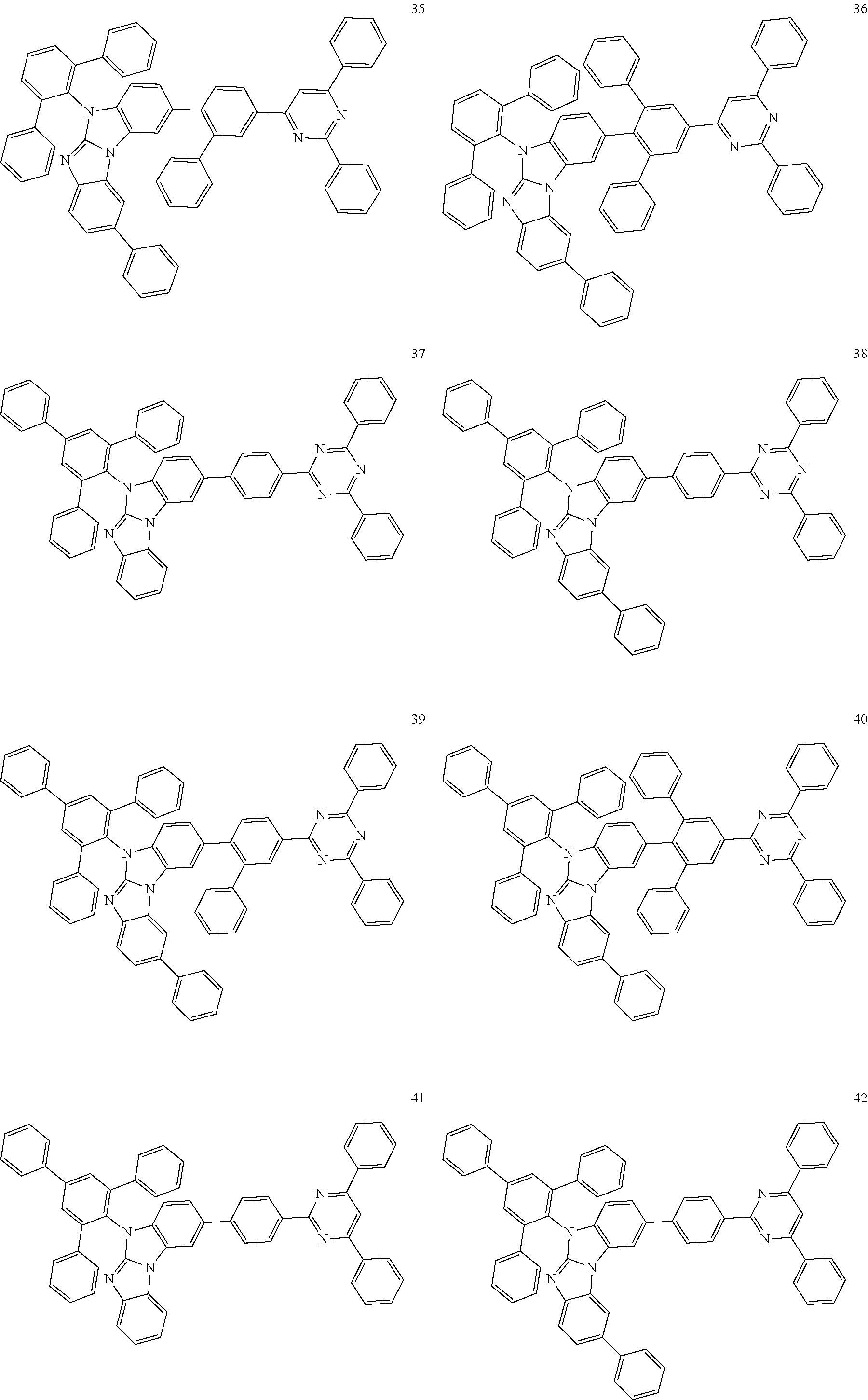

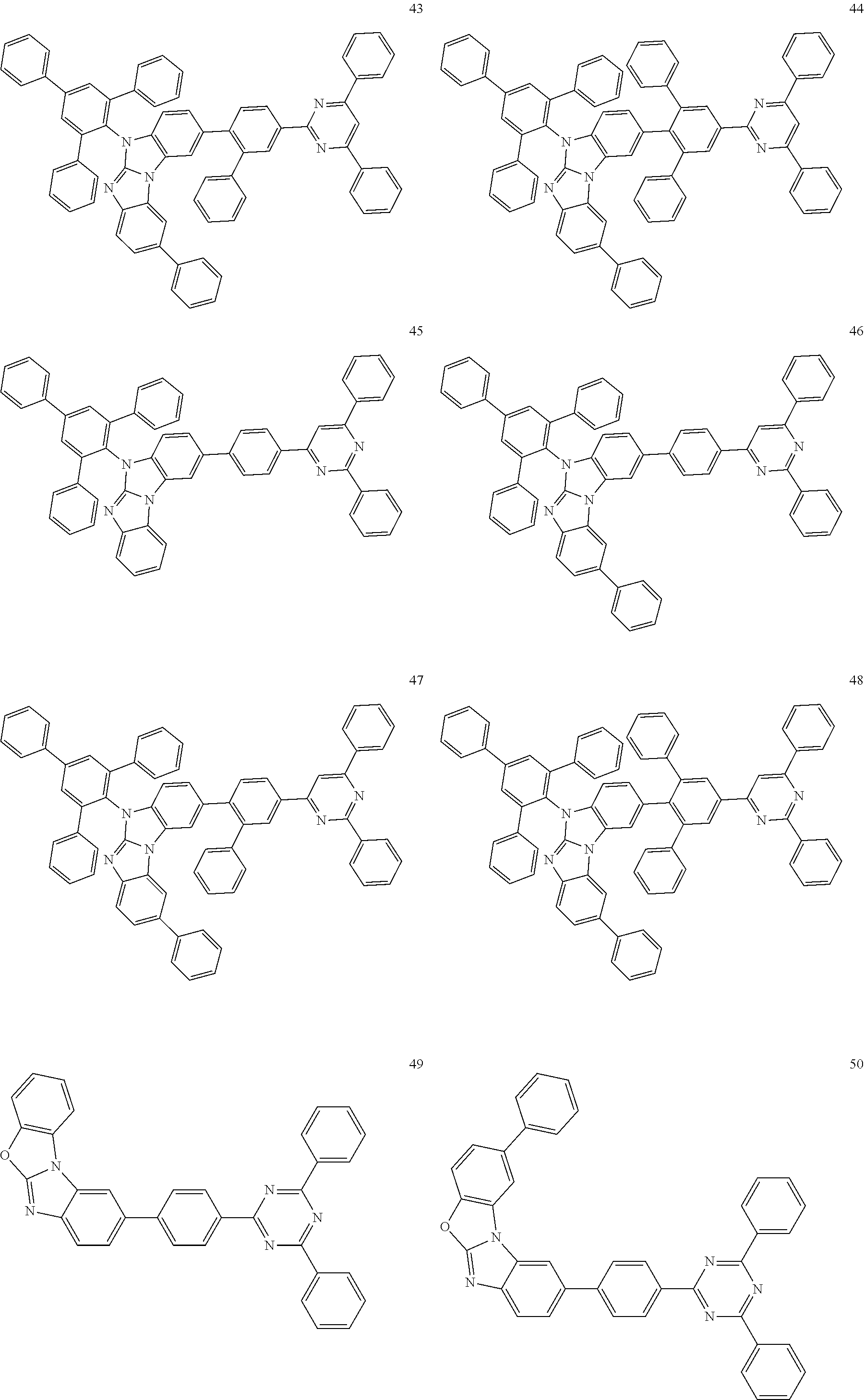

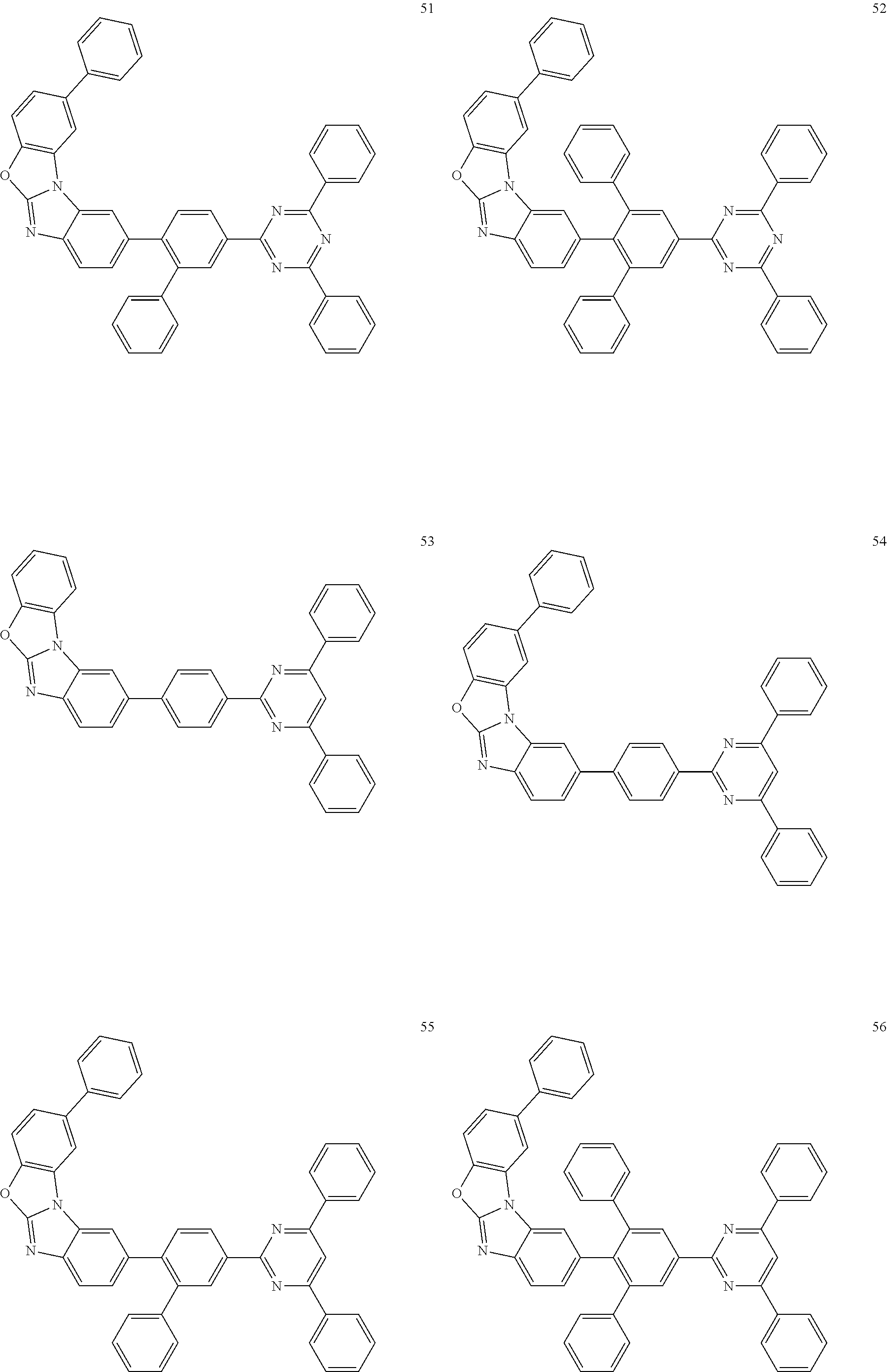

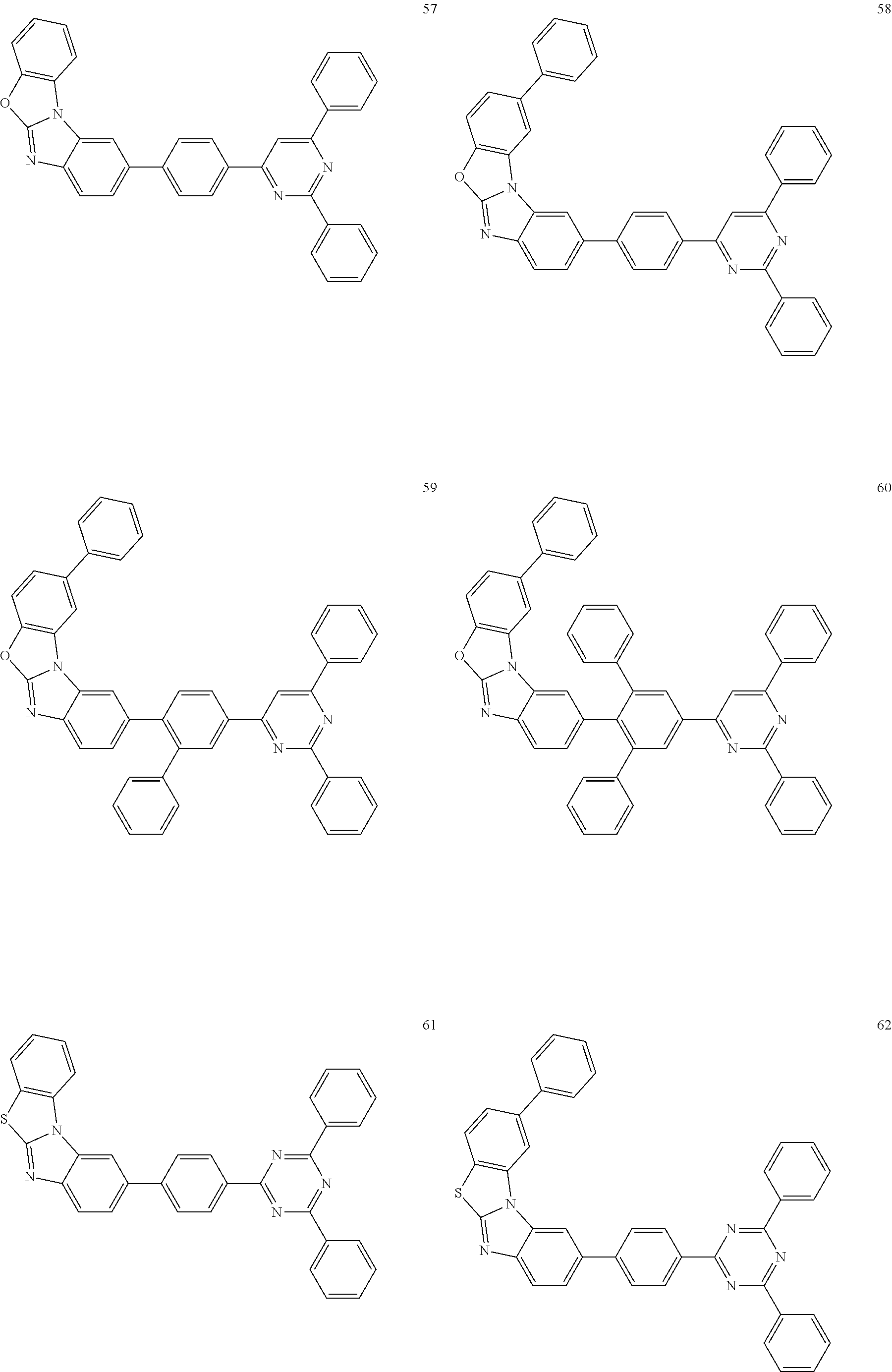 [SMILES: c1ccc(-c2cc(-c3ccc(-c4ccc5nc6oc7ccccc7n6c5c4)cc3)nc(-c3ccccc3)n2)cc1 ; c1ccc(-c2ccc3oc4nc5ccc(-c6ccc(-c7cc(-c8ccccc8)nc(-c8ccccc8)n7)cc6)cc5n4c3c2)cc1 ; c1ccc(-c2ccc3oc4nc5ccc(-c6ccc(-c7cc(-c8ccccc8)nc(-c8ccccc8)n7)cc6-c6ccccc6)cc5n4c3c2)cc1 ; c1ccc(-c2ccc3oc4nc5ccc(-c6c(-c7ccccc7)cc(-c7cc(-c8ccccc8)nc(-c8ccccc8)n7)cc6-c6ccccc6)cc5n4c3c2)cc1 ; c1ccc(-c2nc(-c3ccccc3)nc(-c3ccc(-c4ccc5nc6sc7ccccc7n6c5c4)cc3)n2)cc1 ; c1ccc(-c2ccc3sc4nc5ccc(-c6ccc(-c7nc(-c8ccccc8)nc(-c8ccccc8)n7)cc6)cc5n4c3c2)cc1]

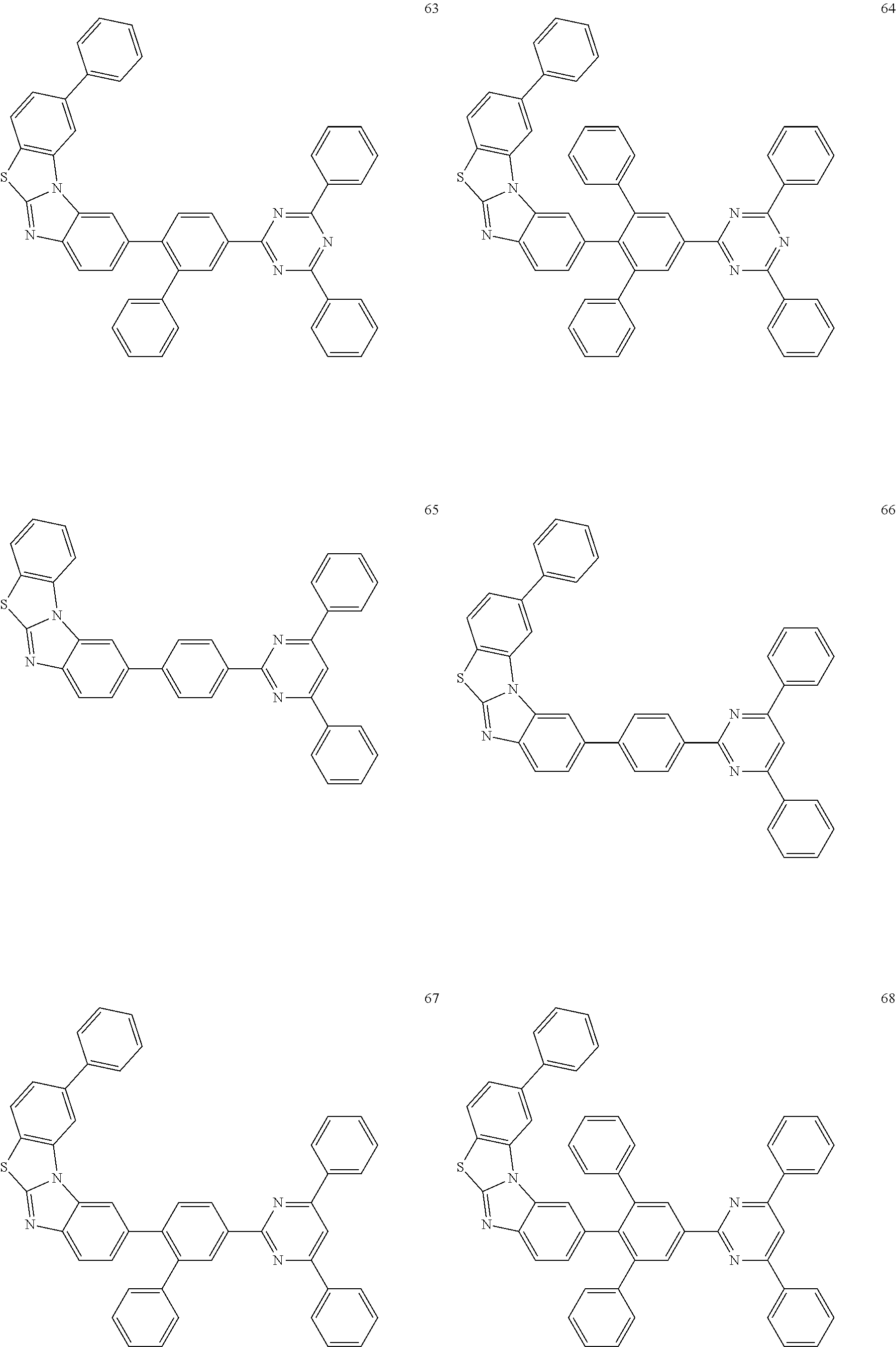

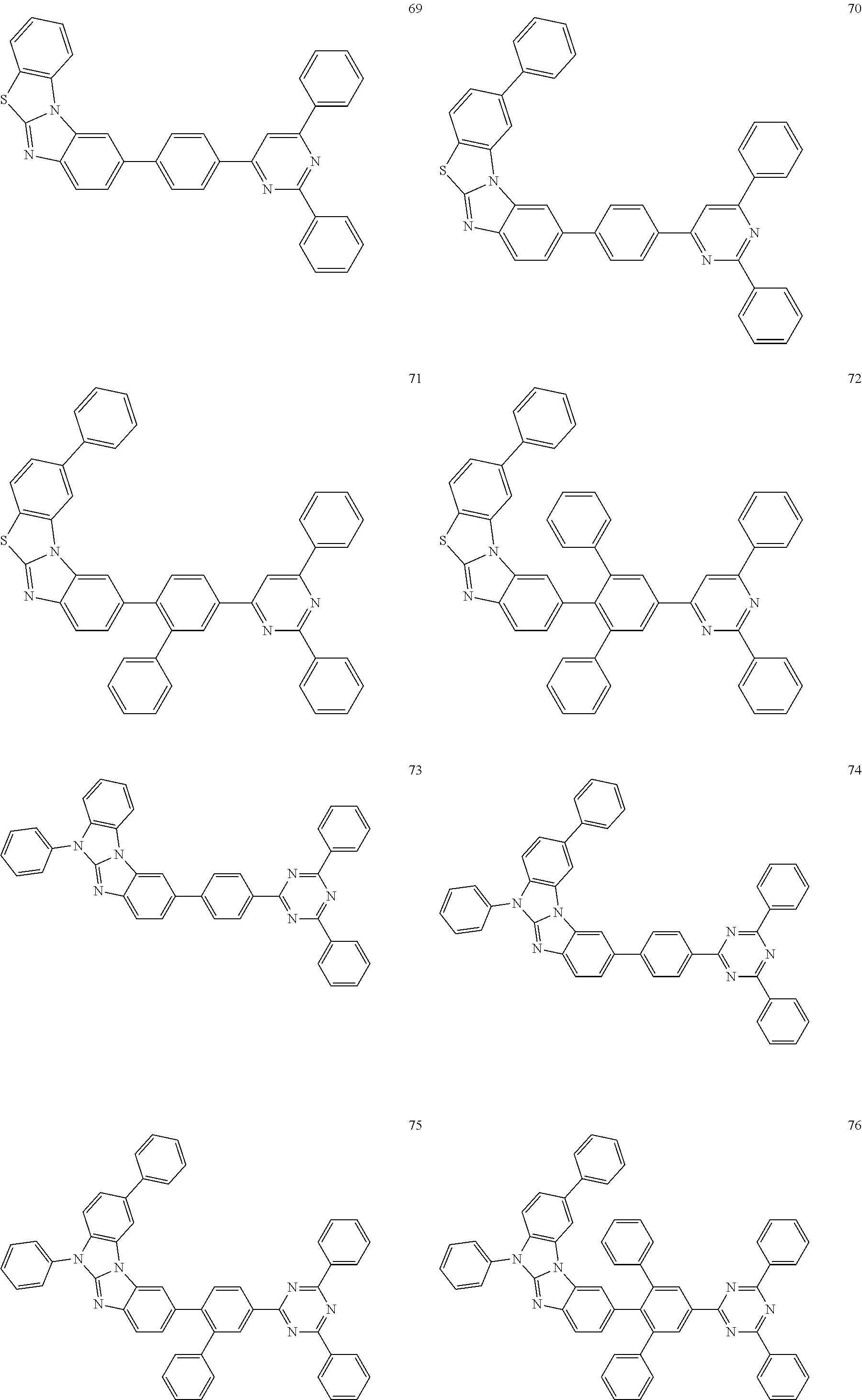

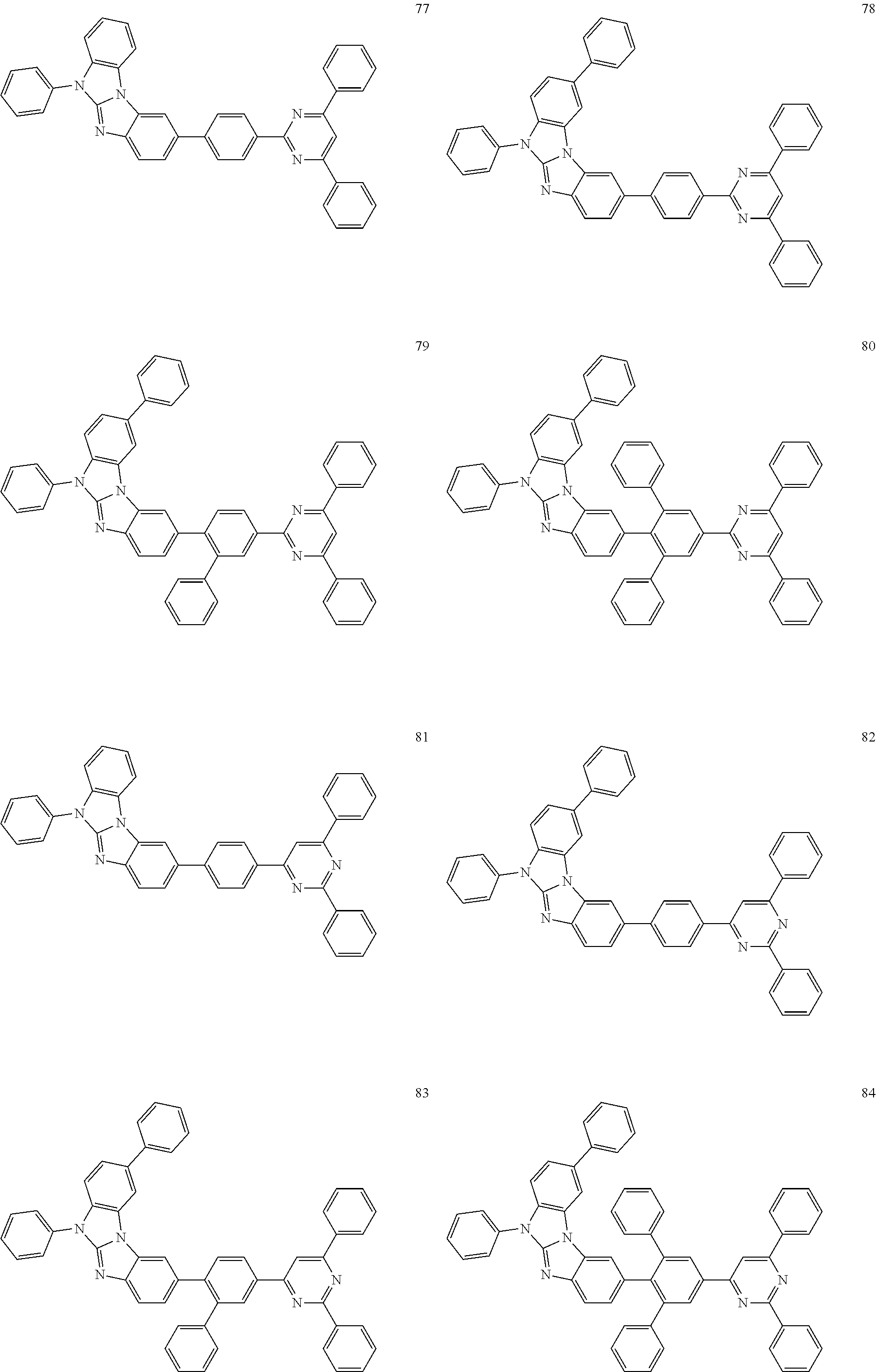

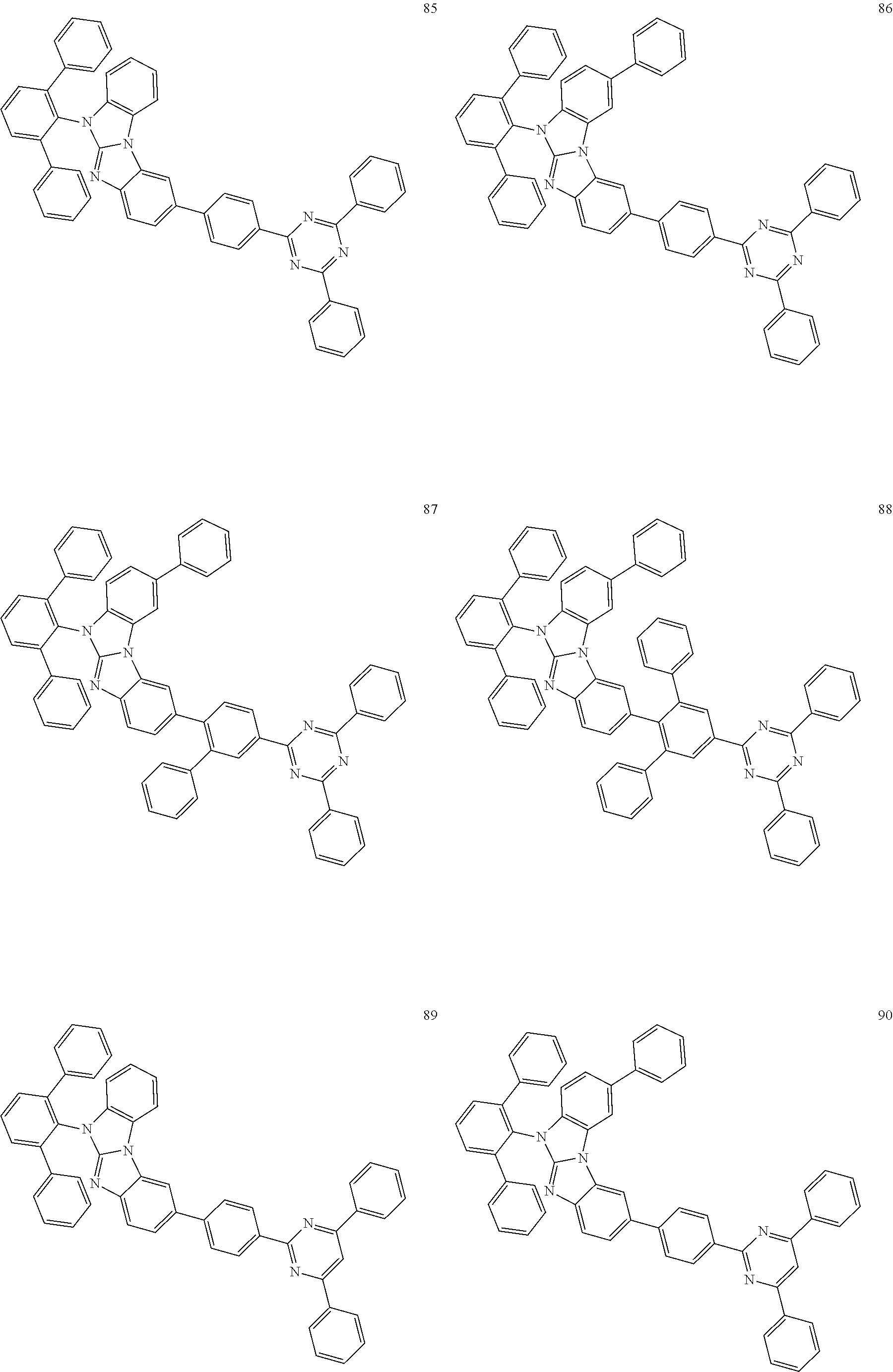

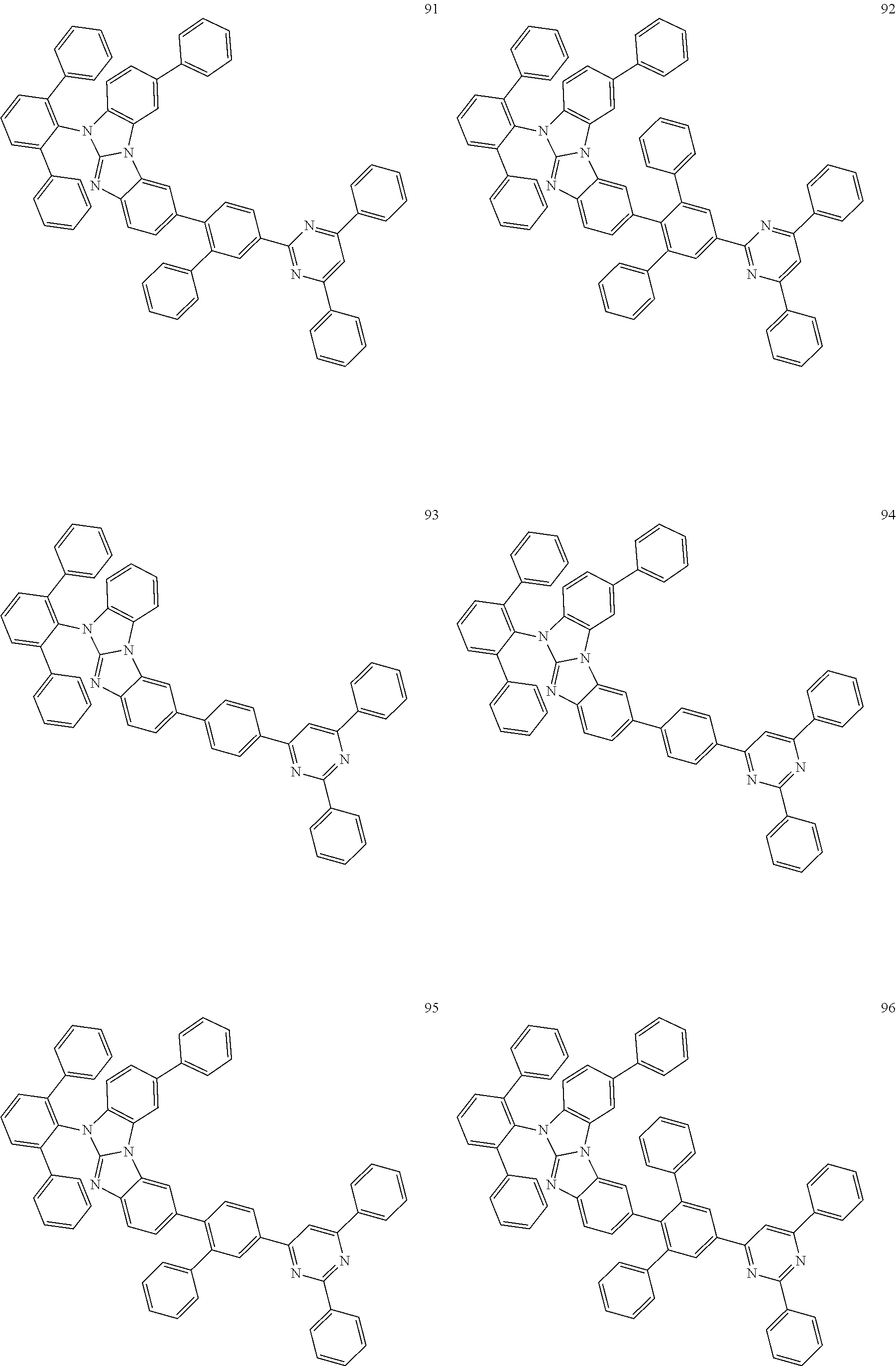

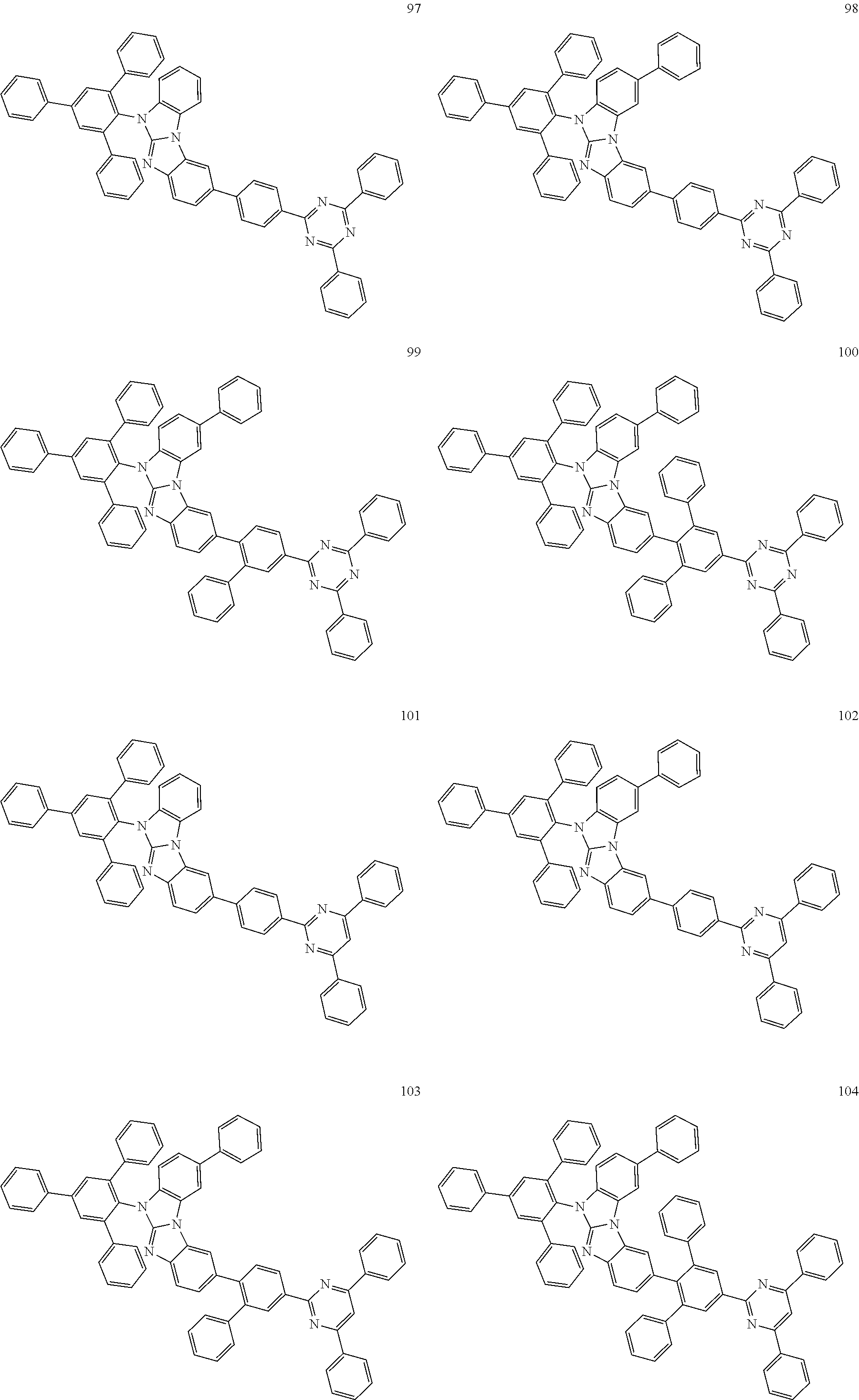

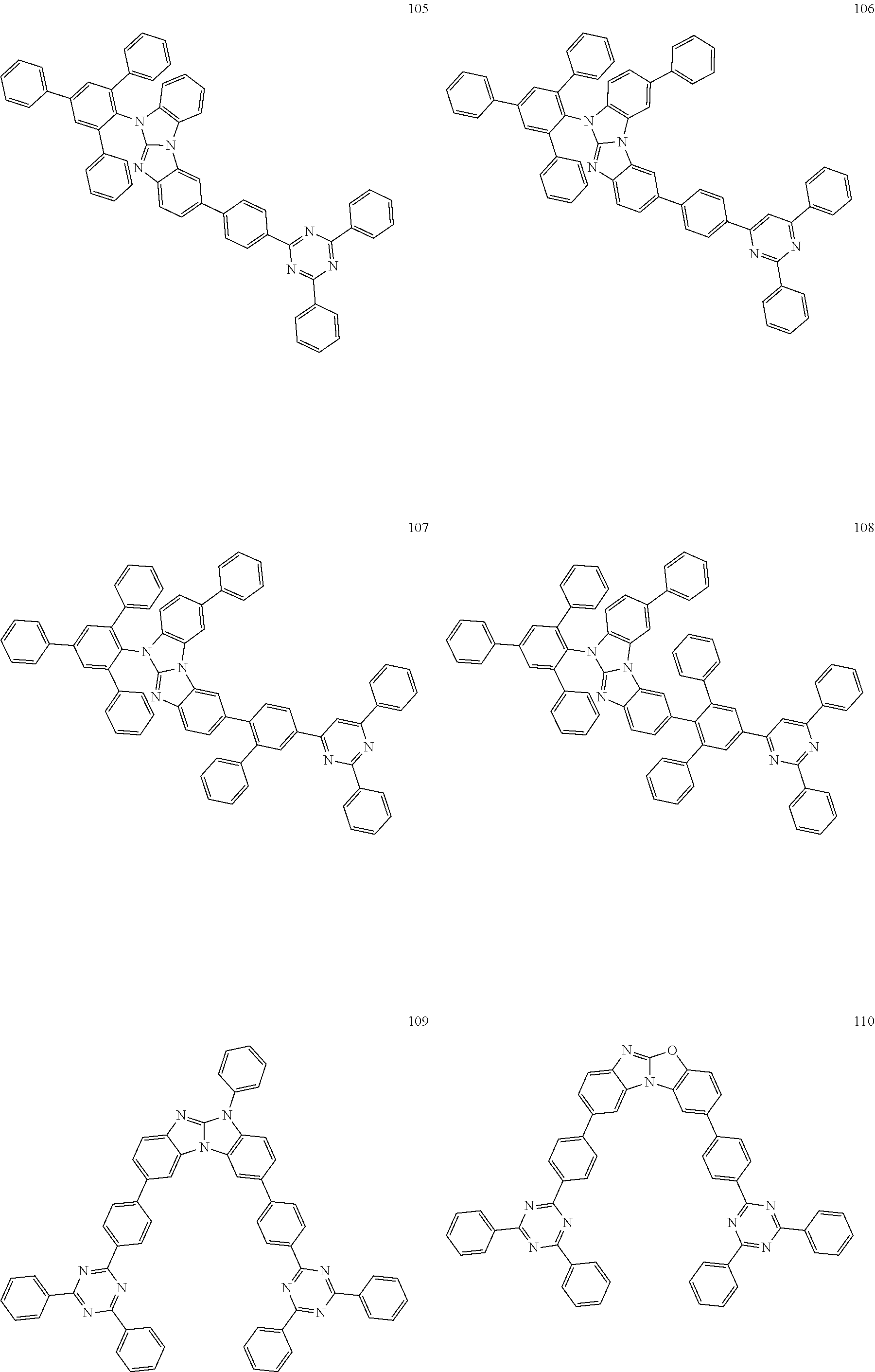

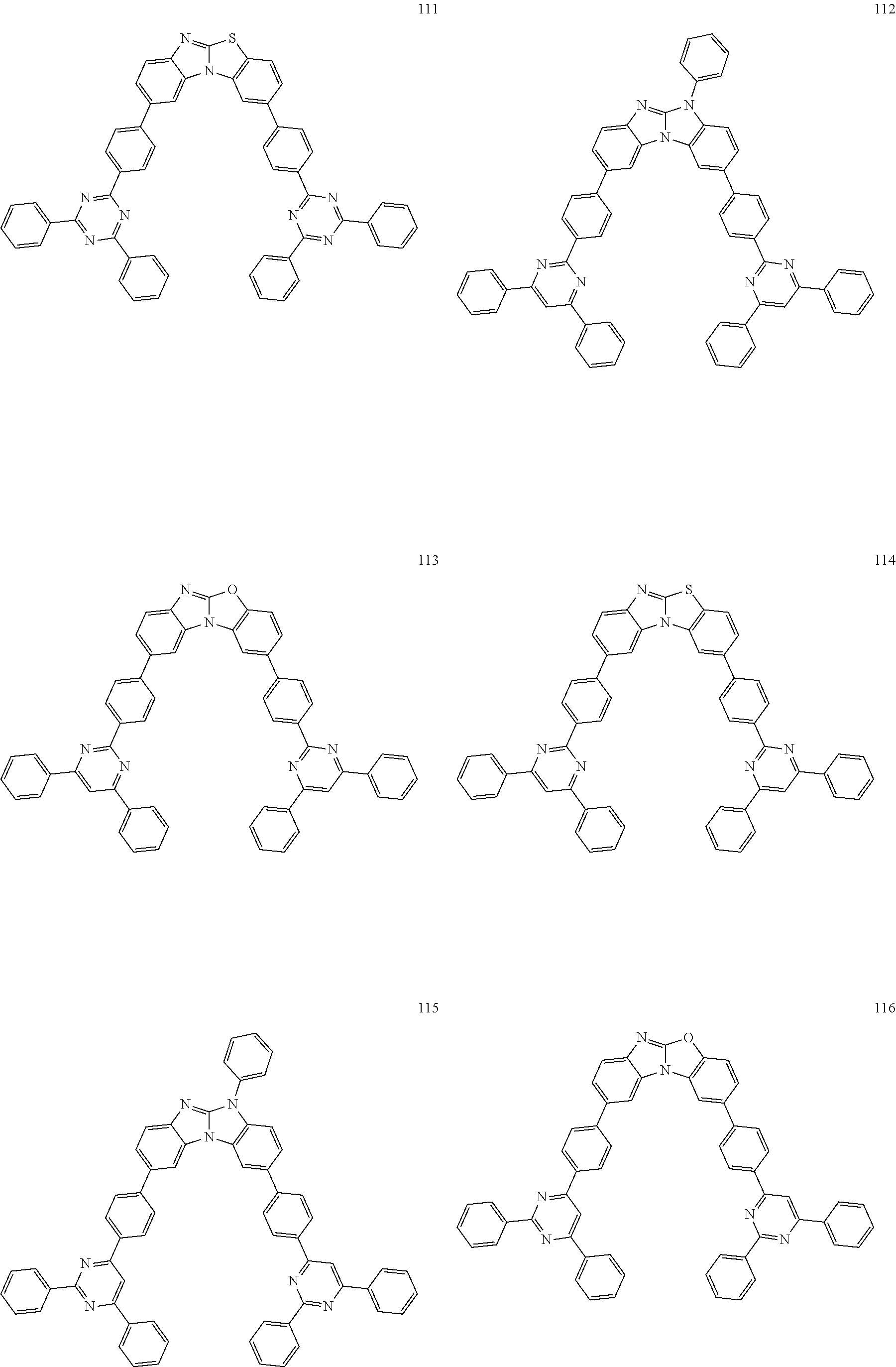

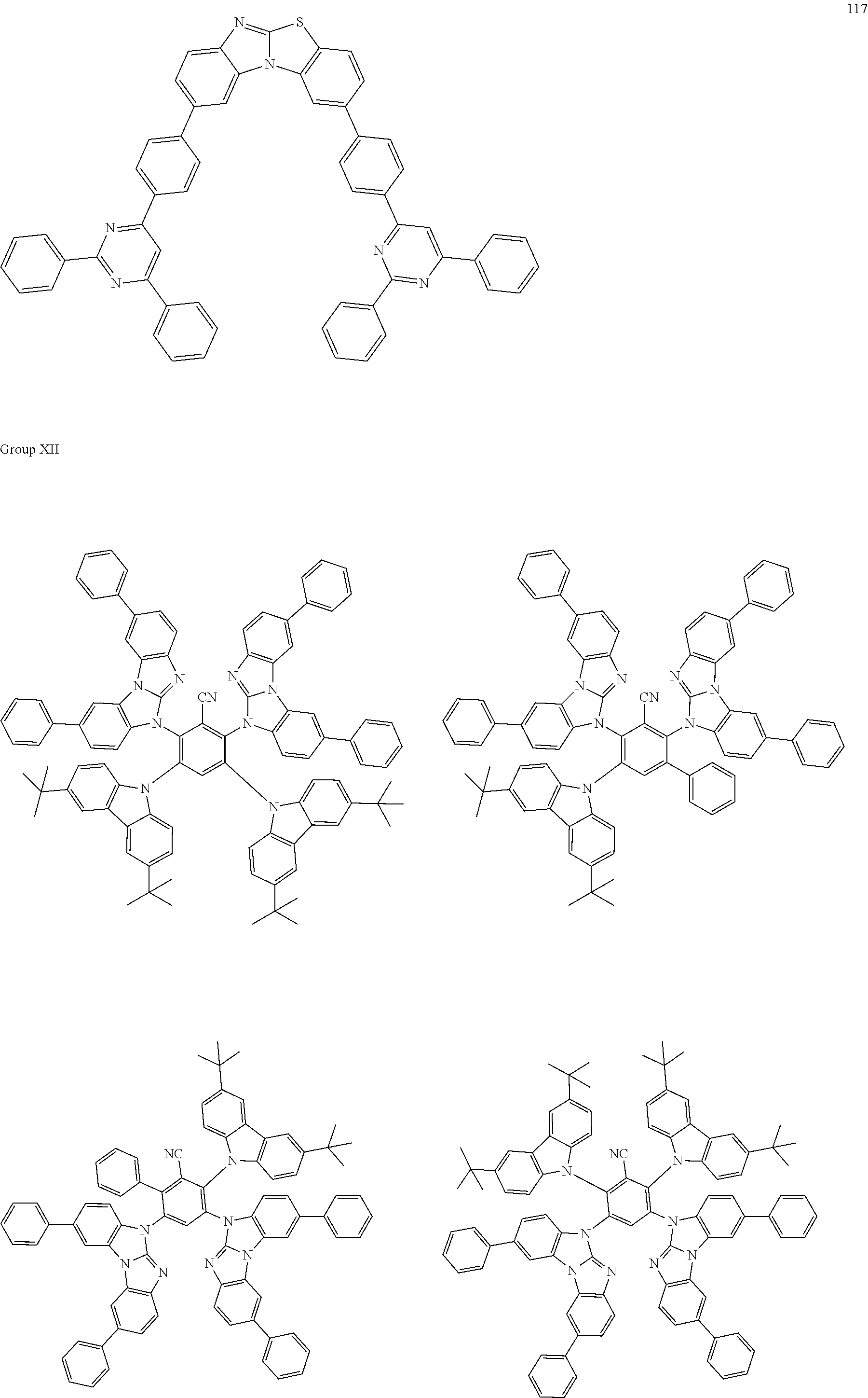

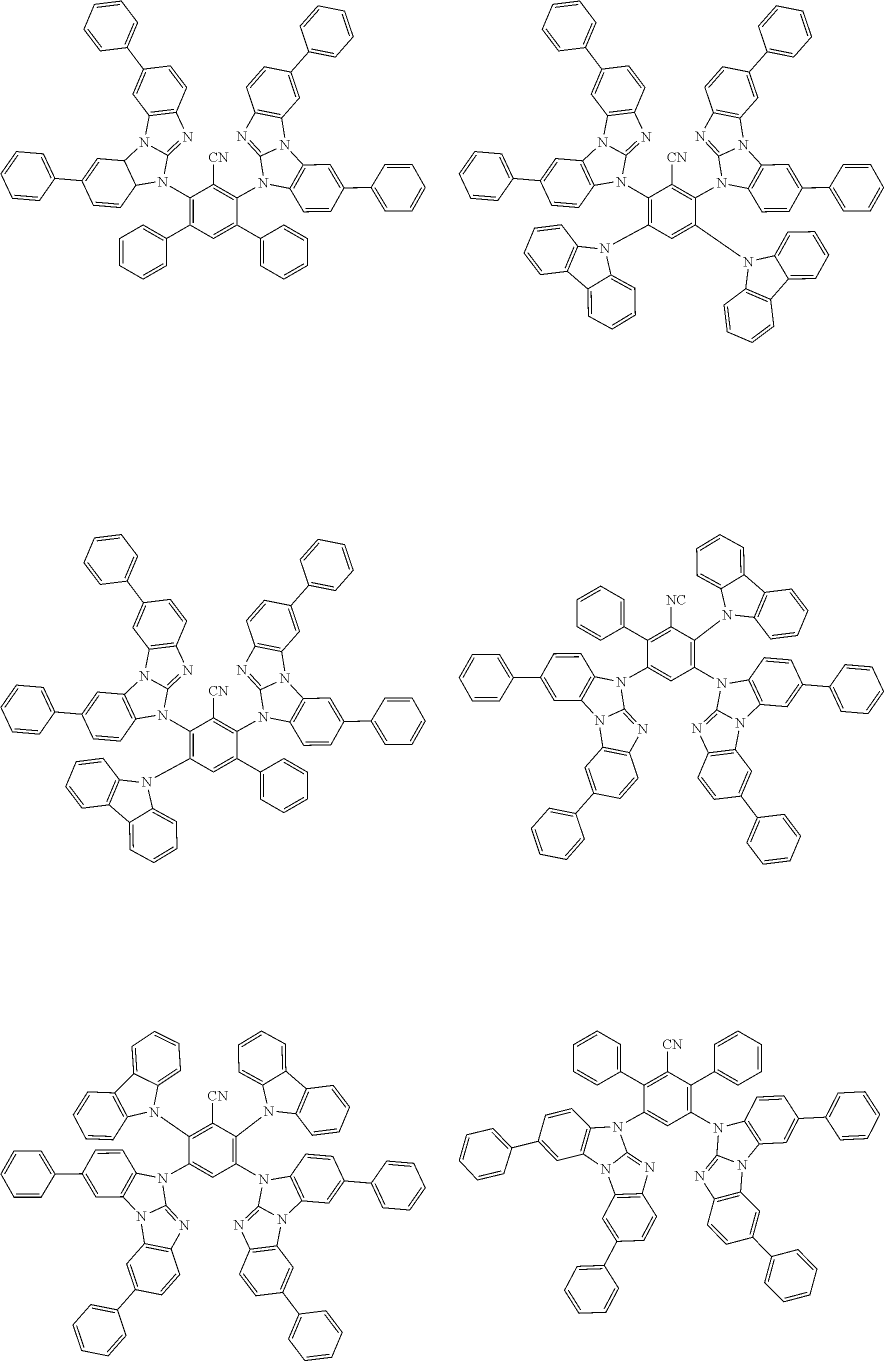

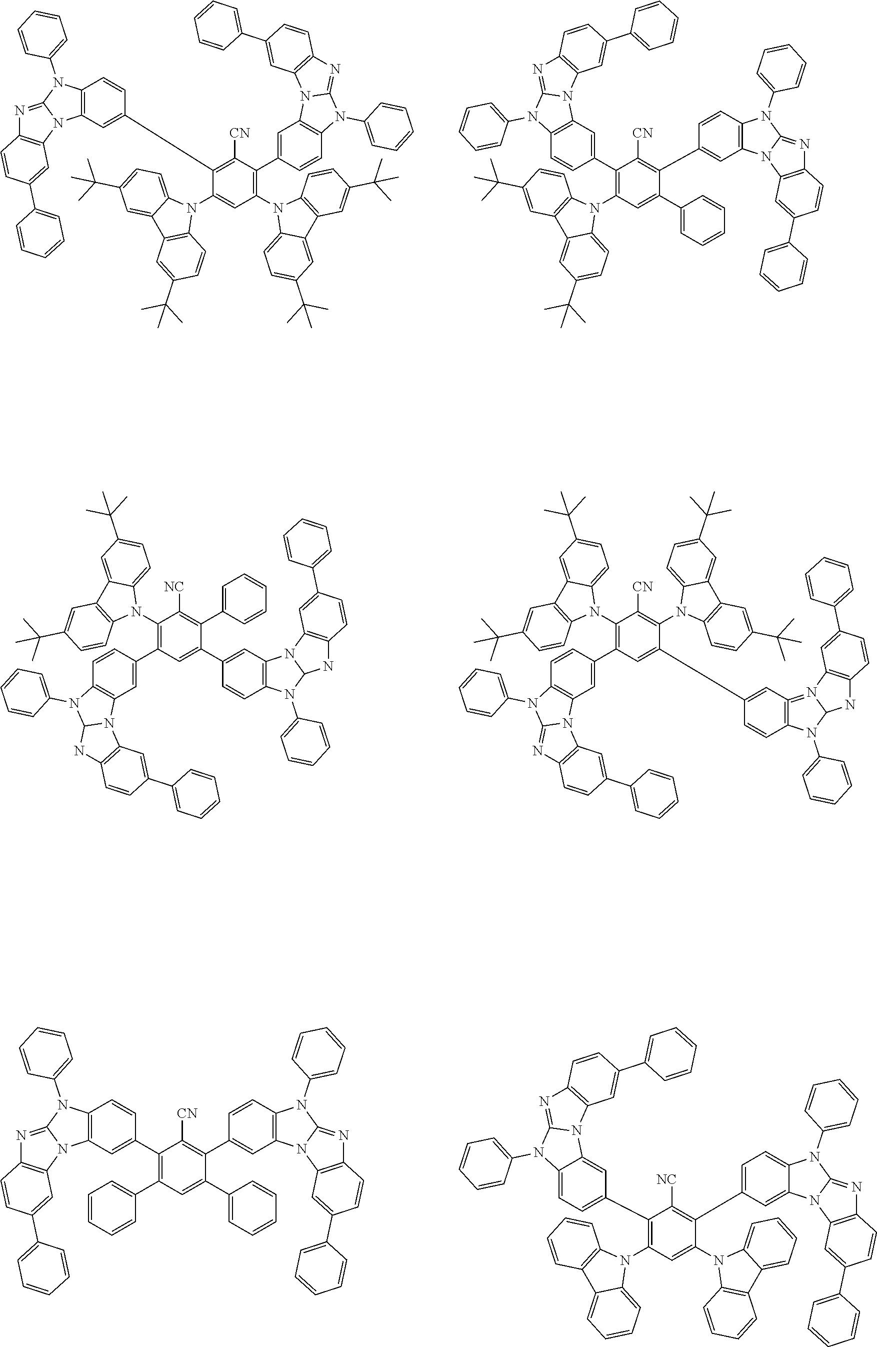

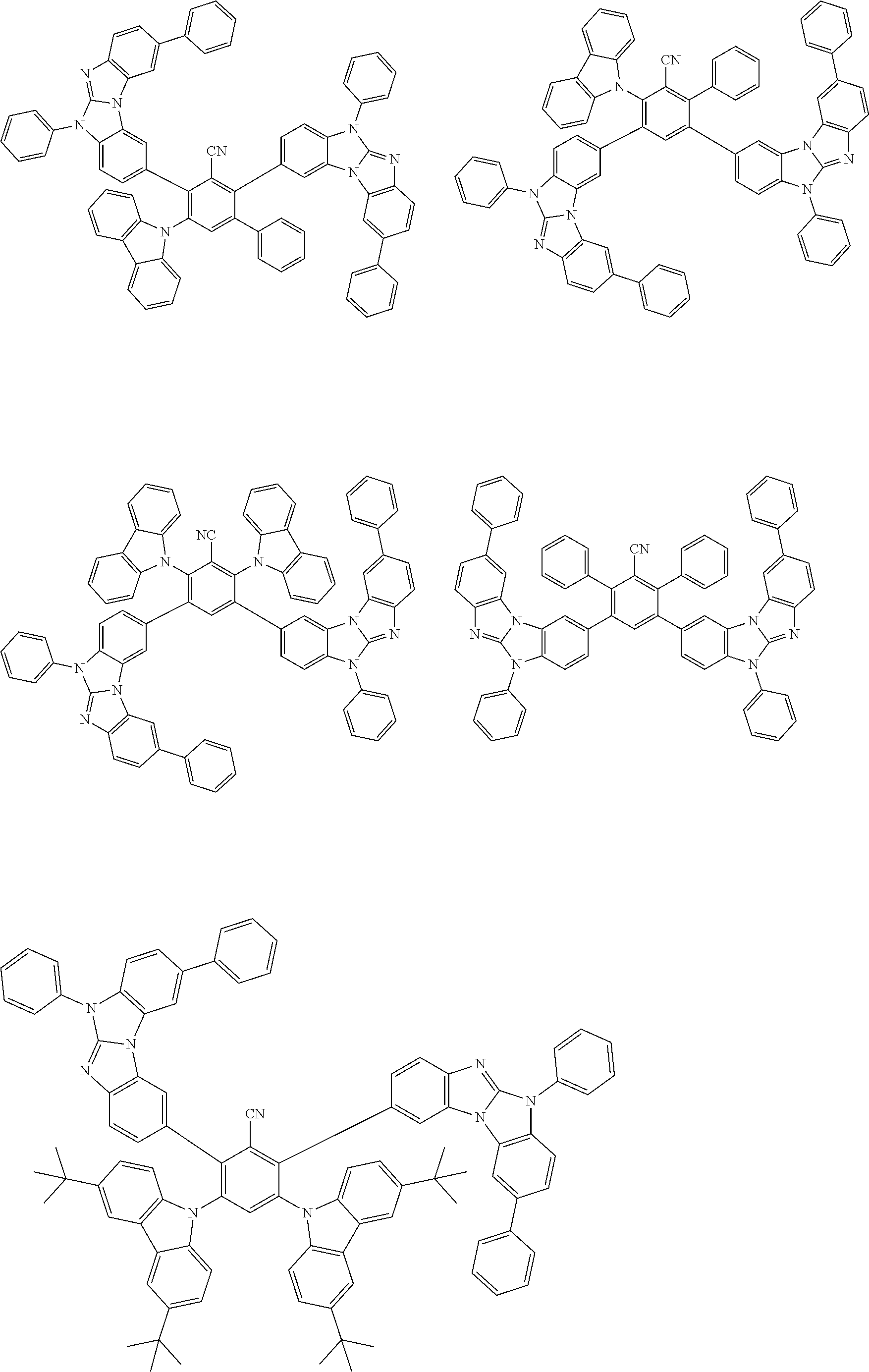

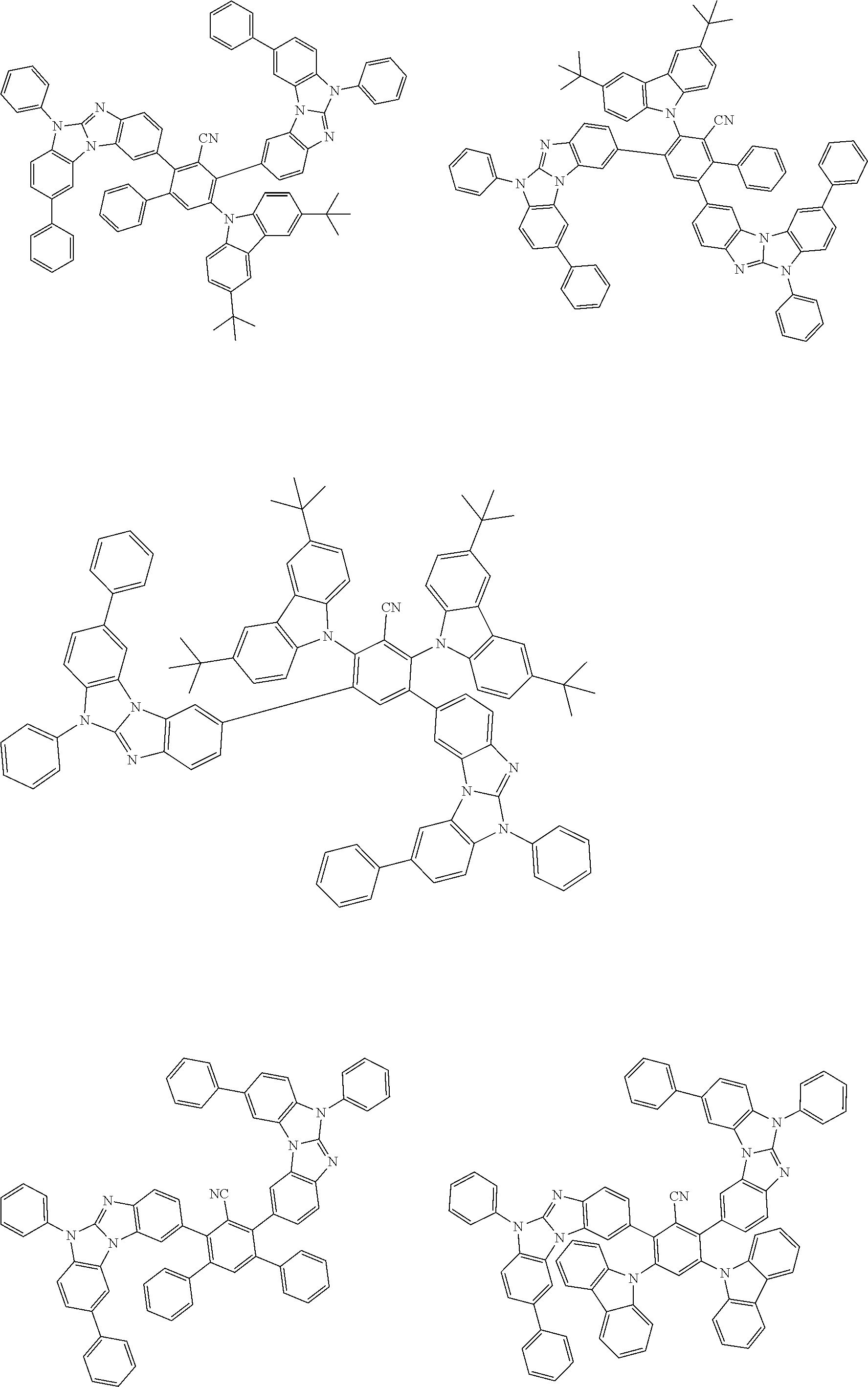

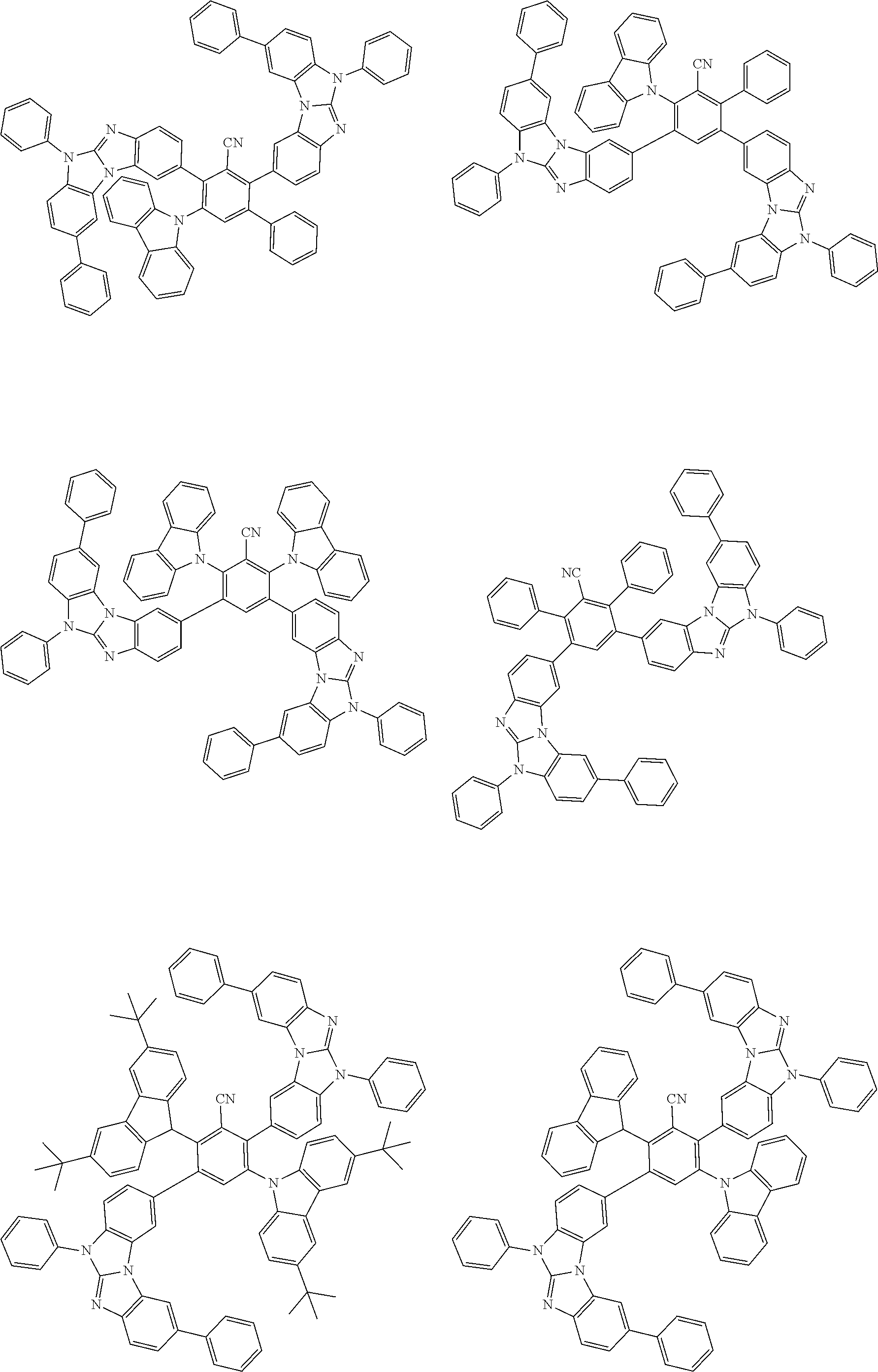

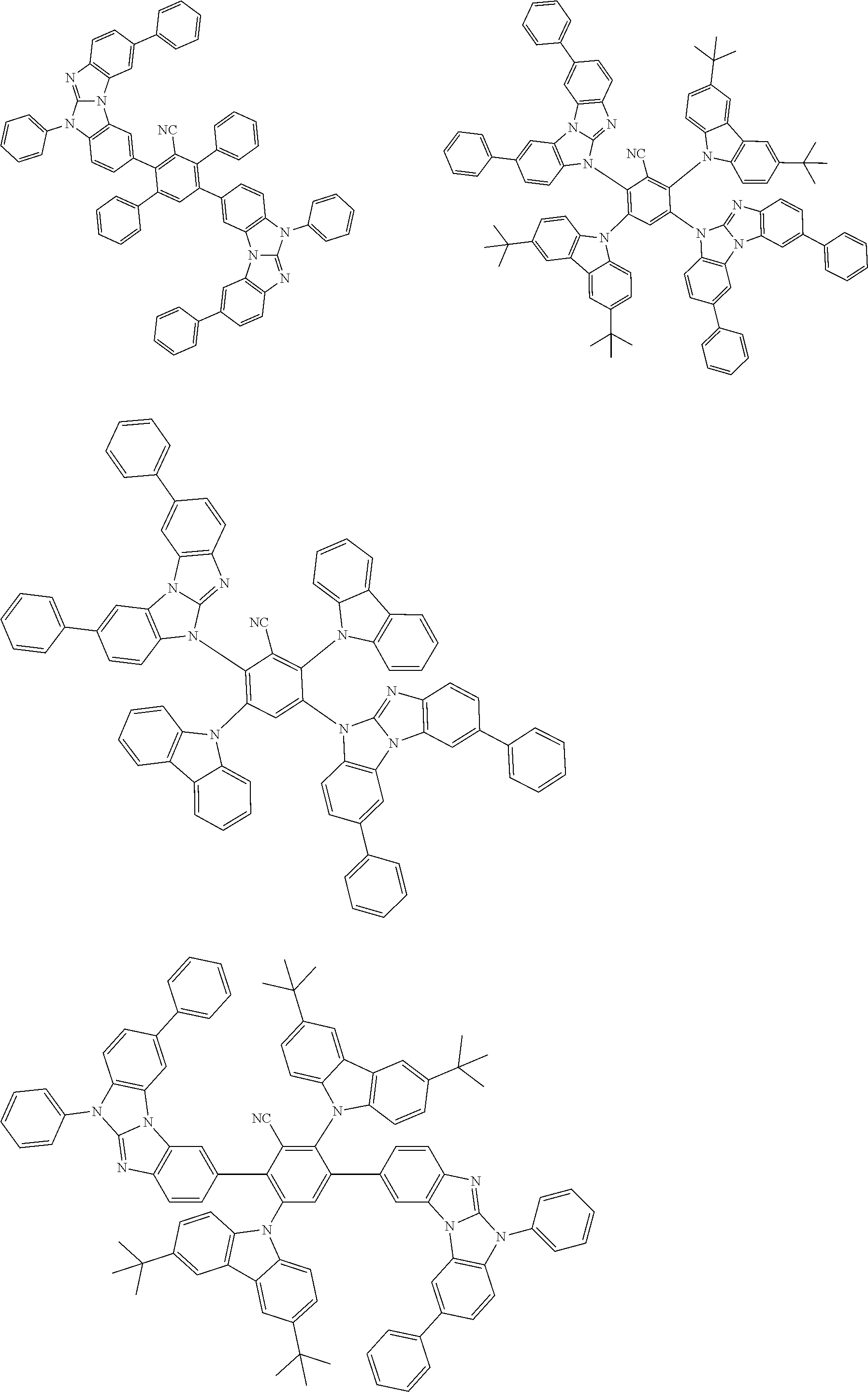

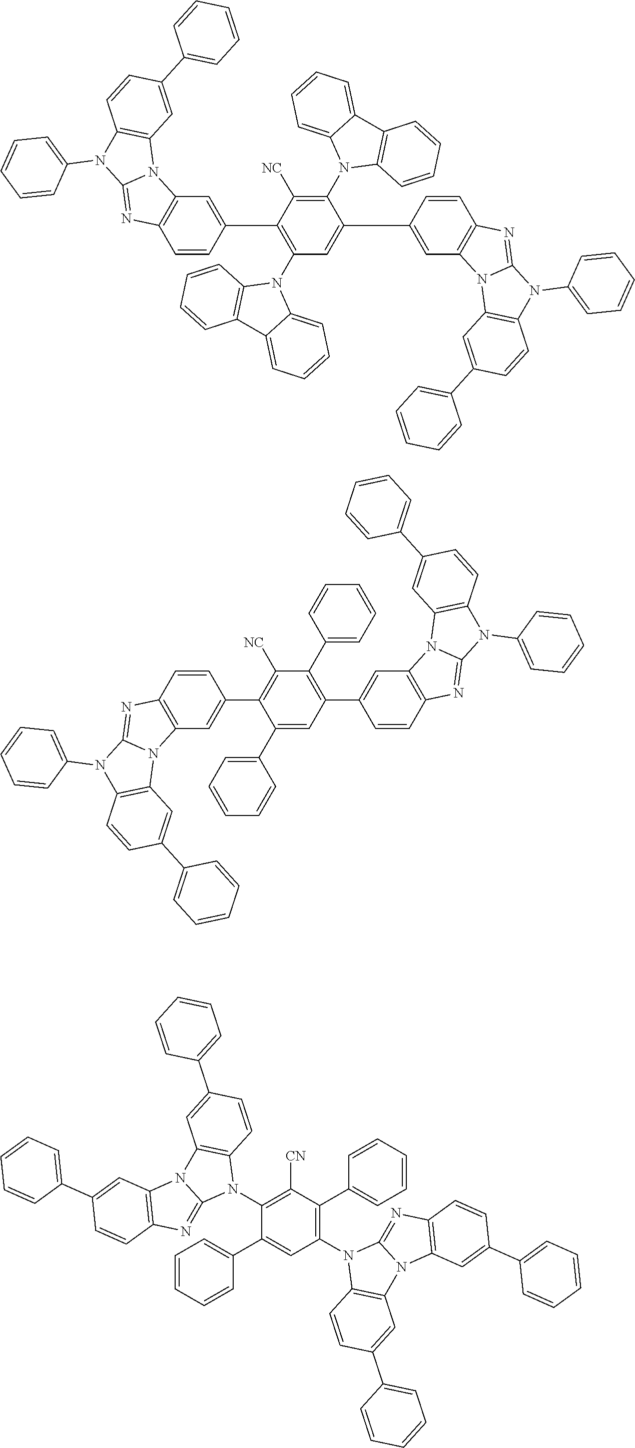

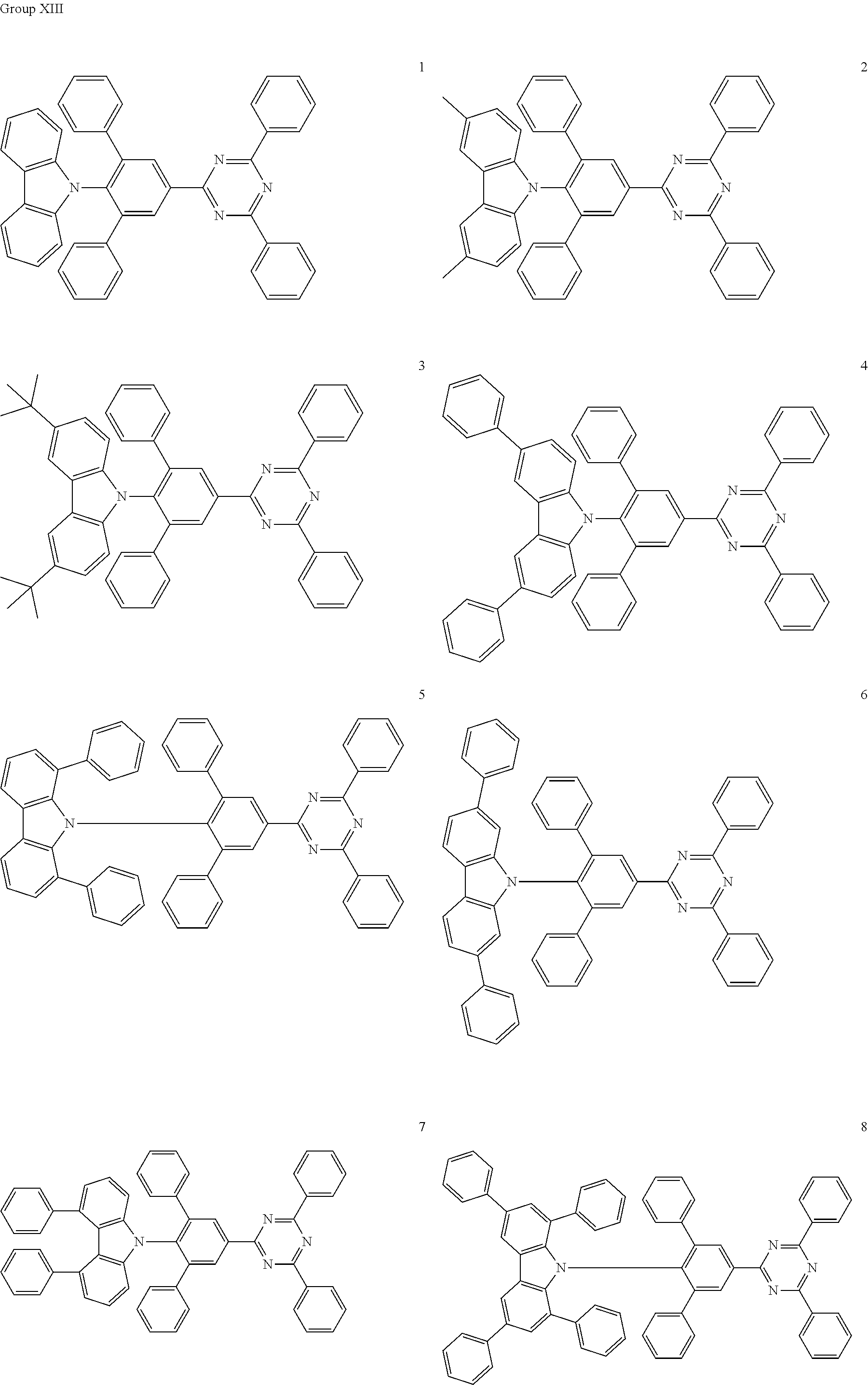

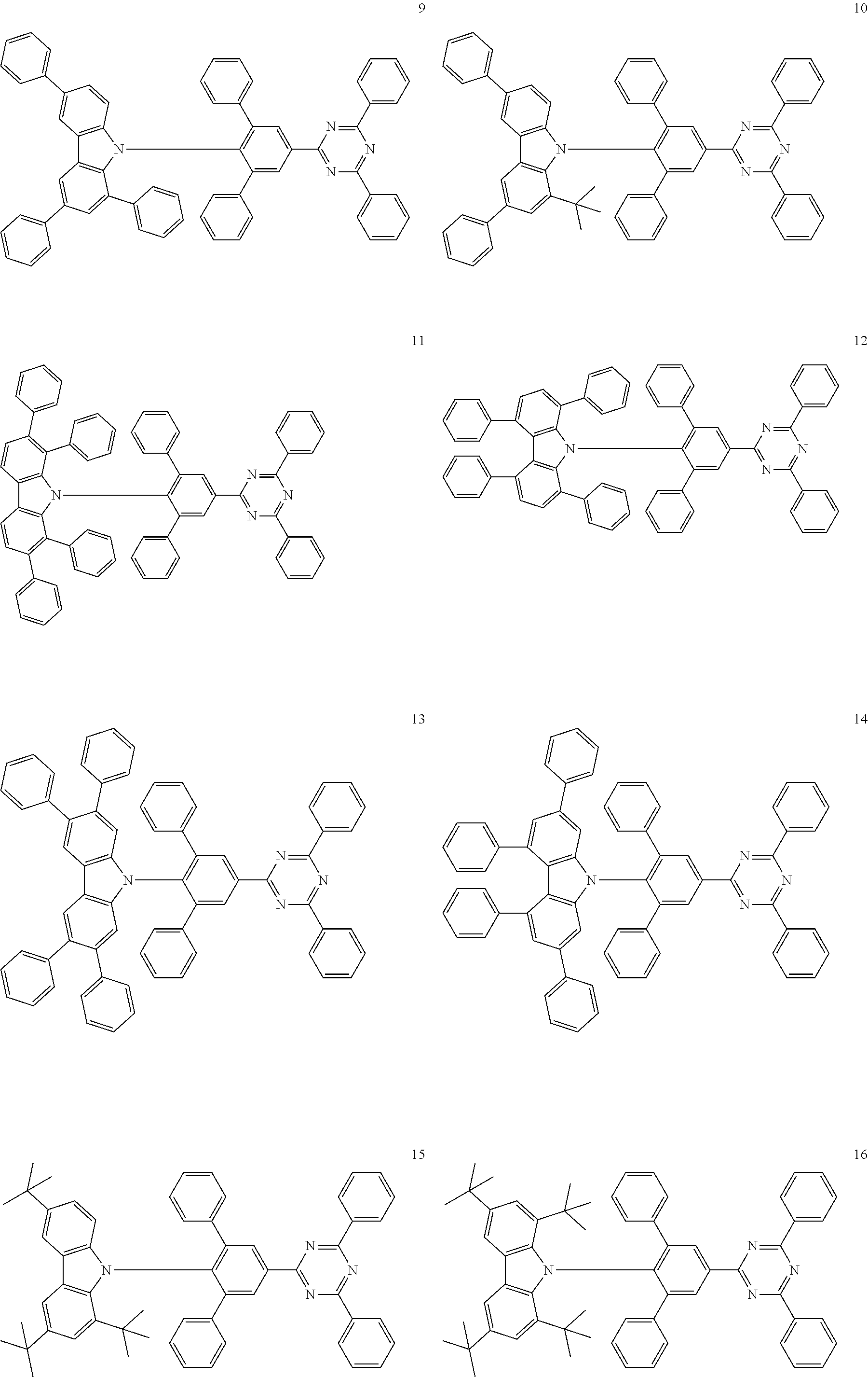

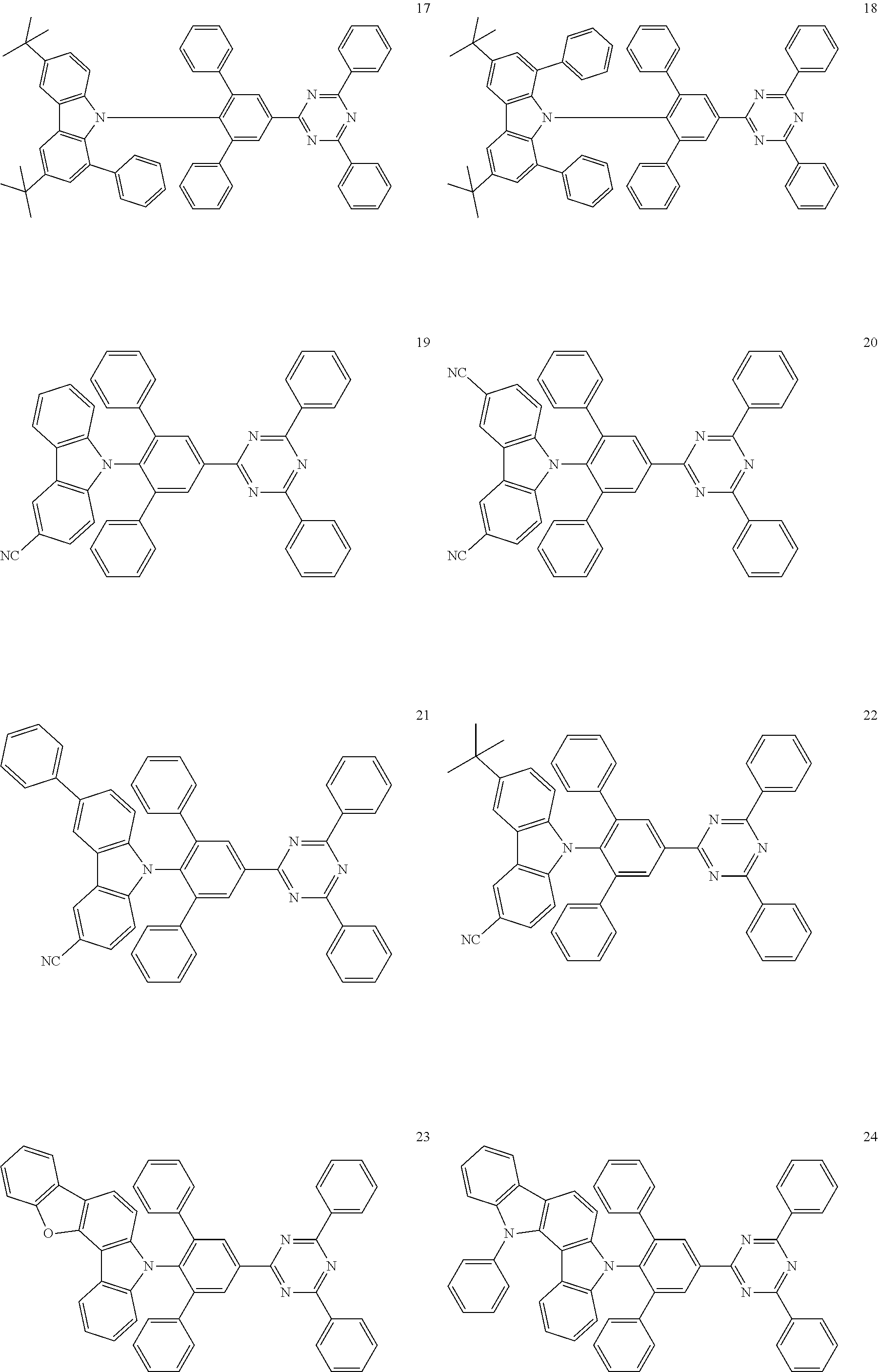

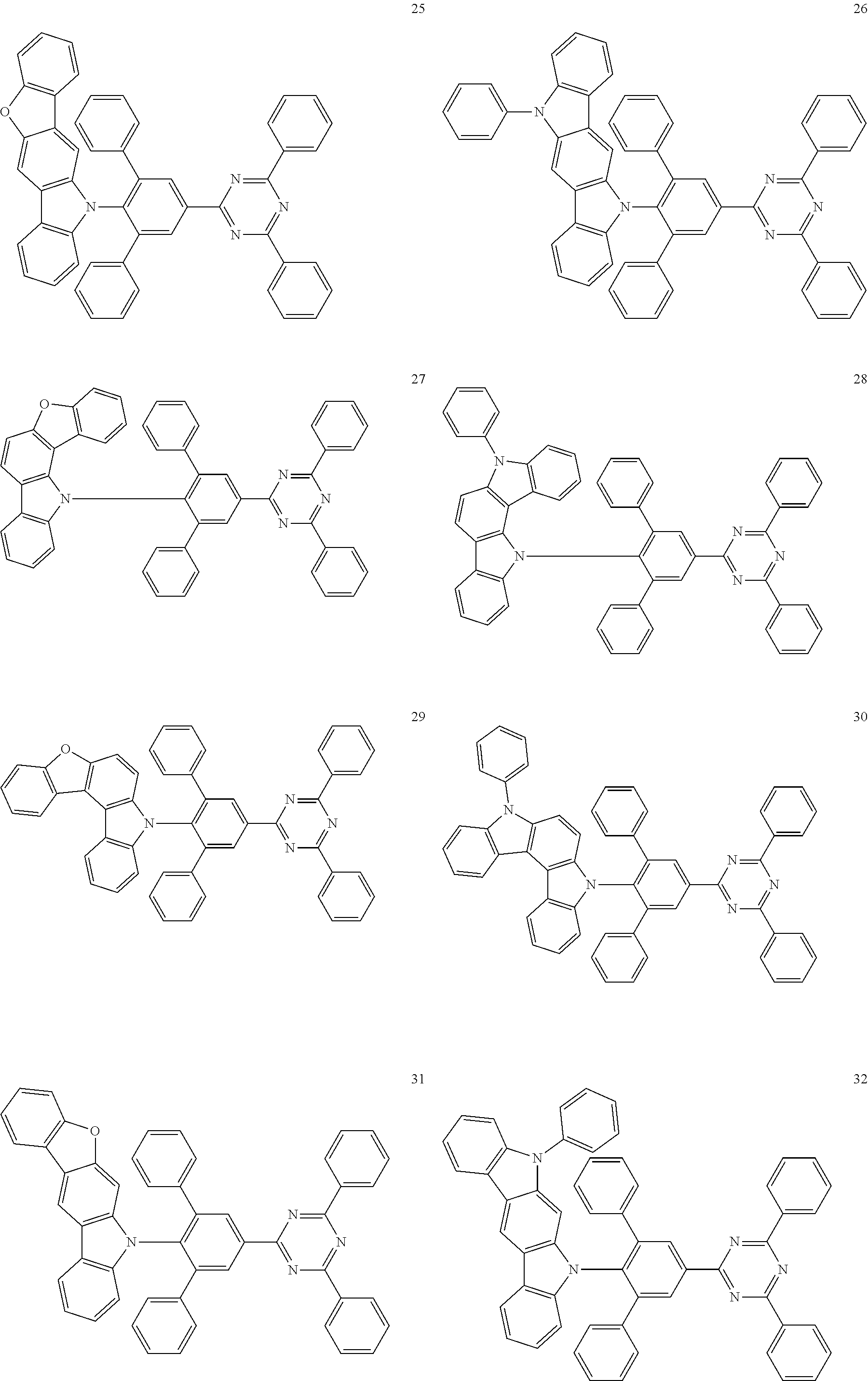

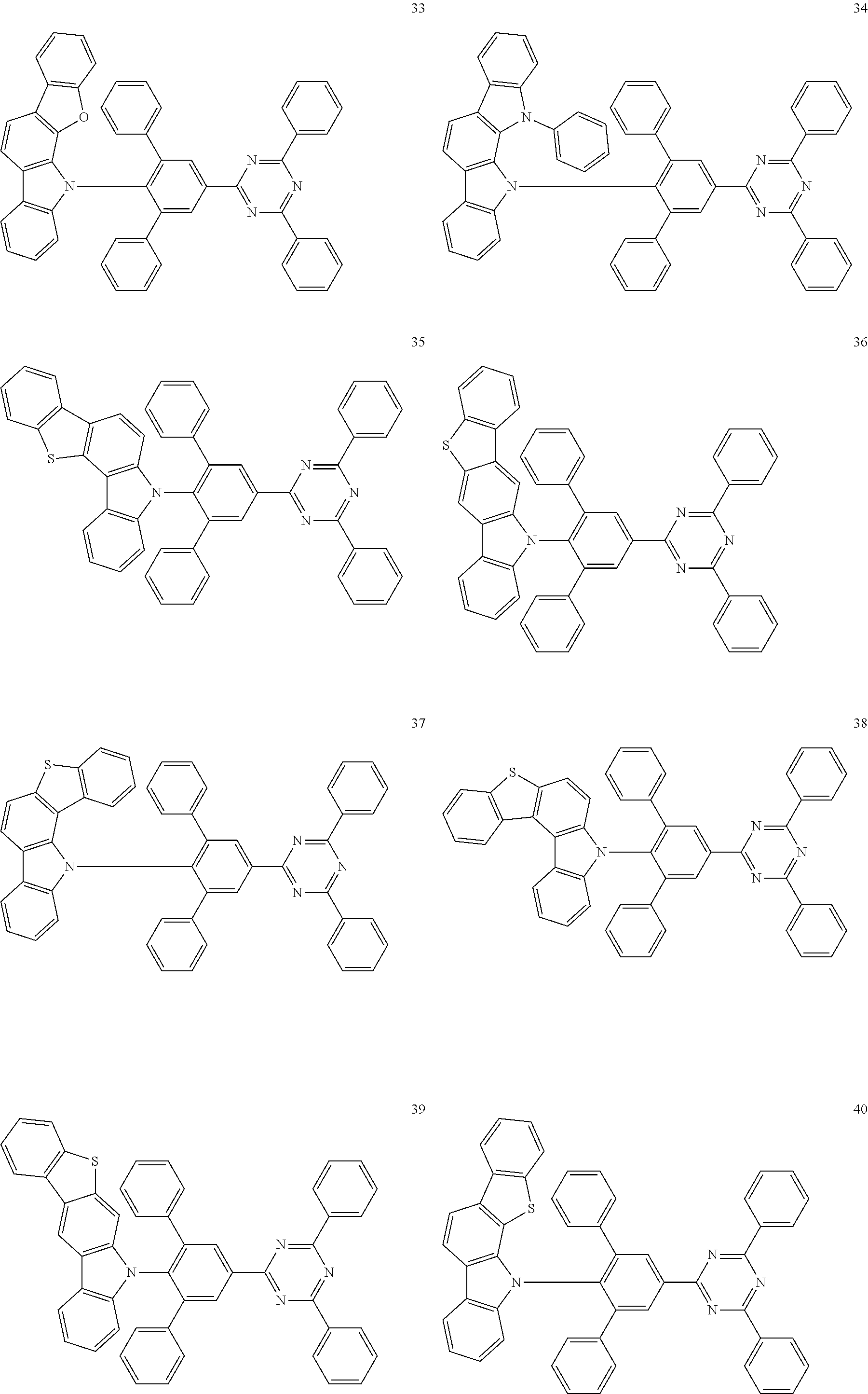

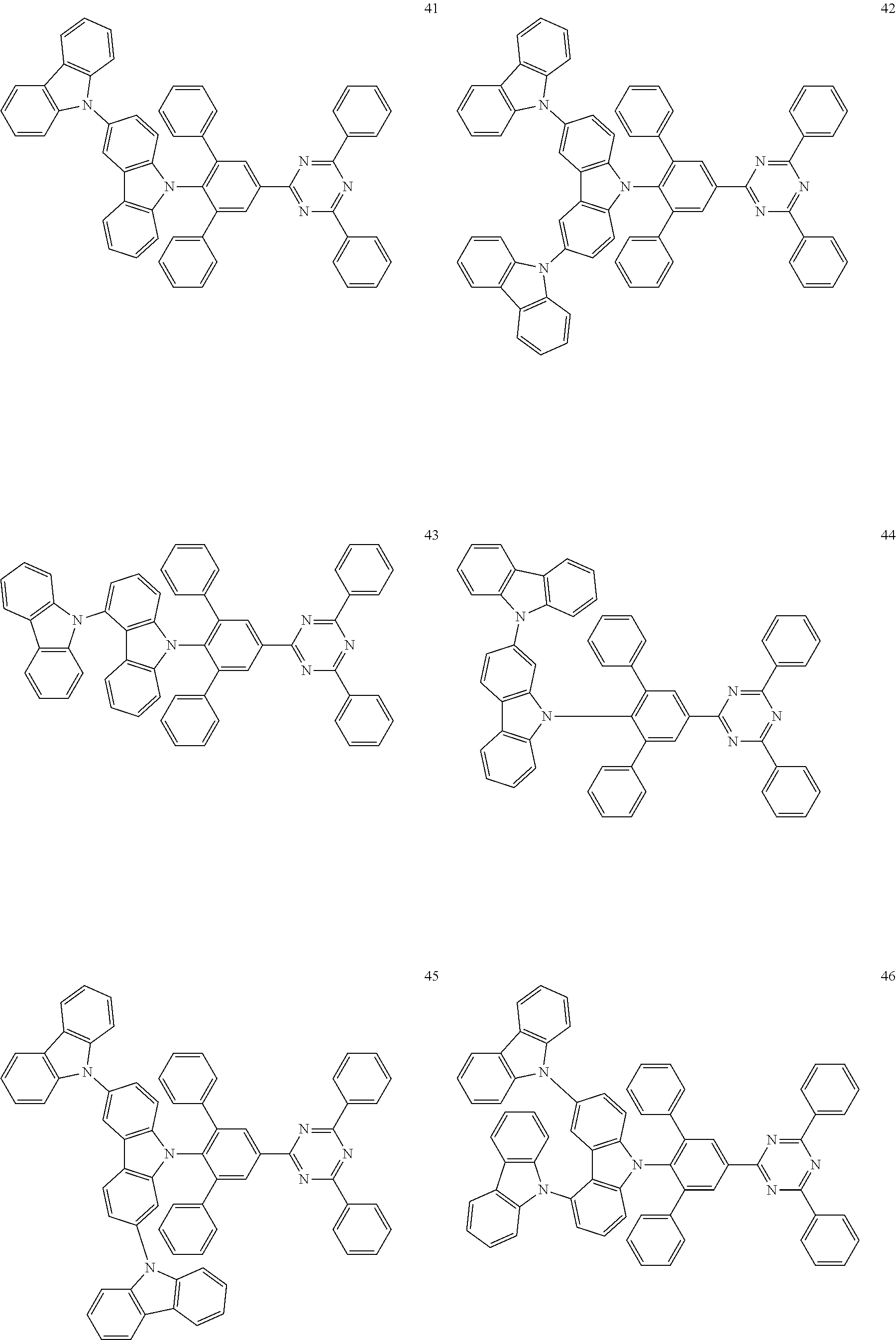

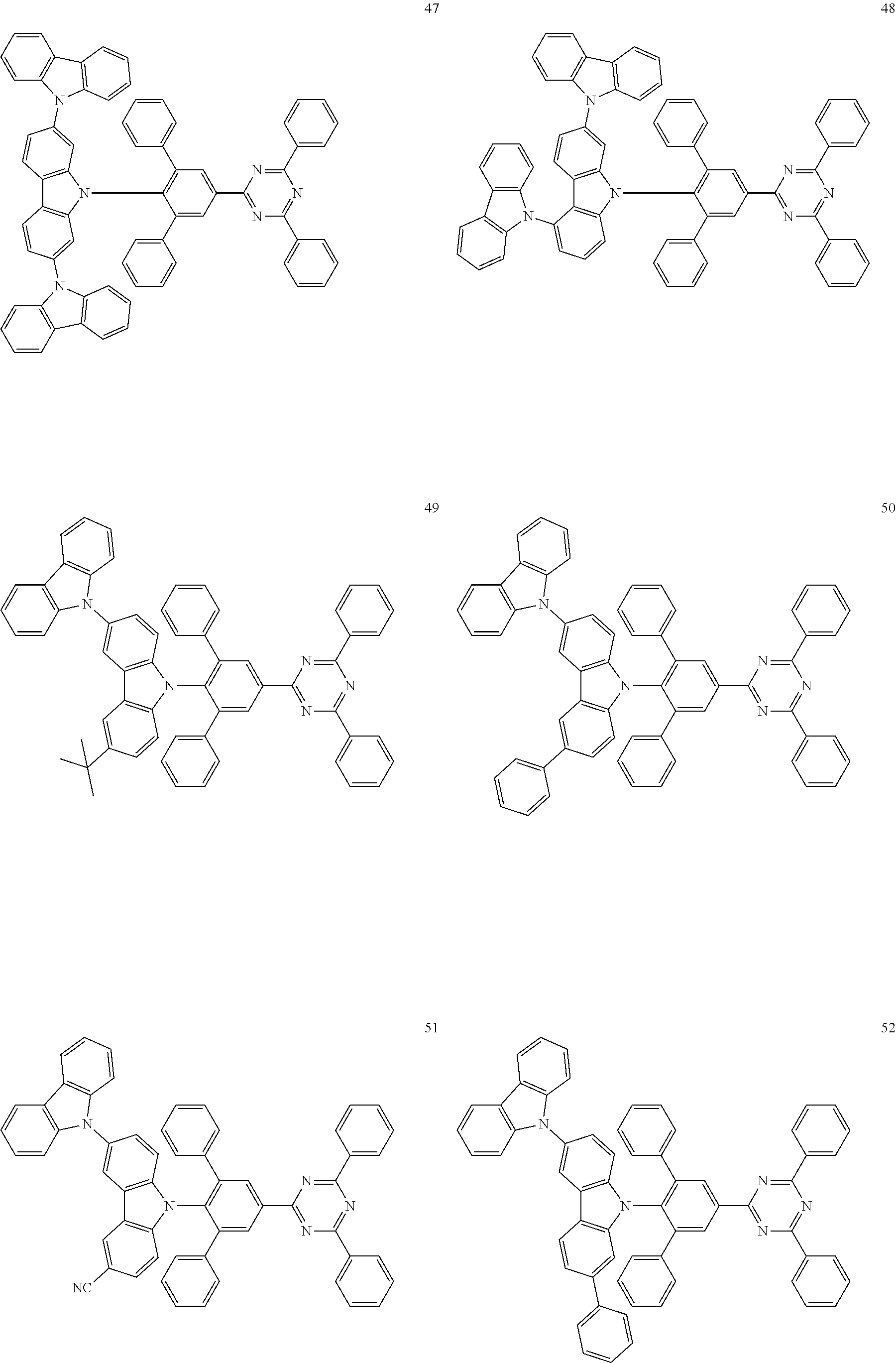

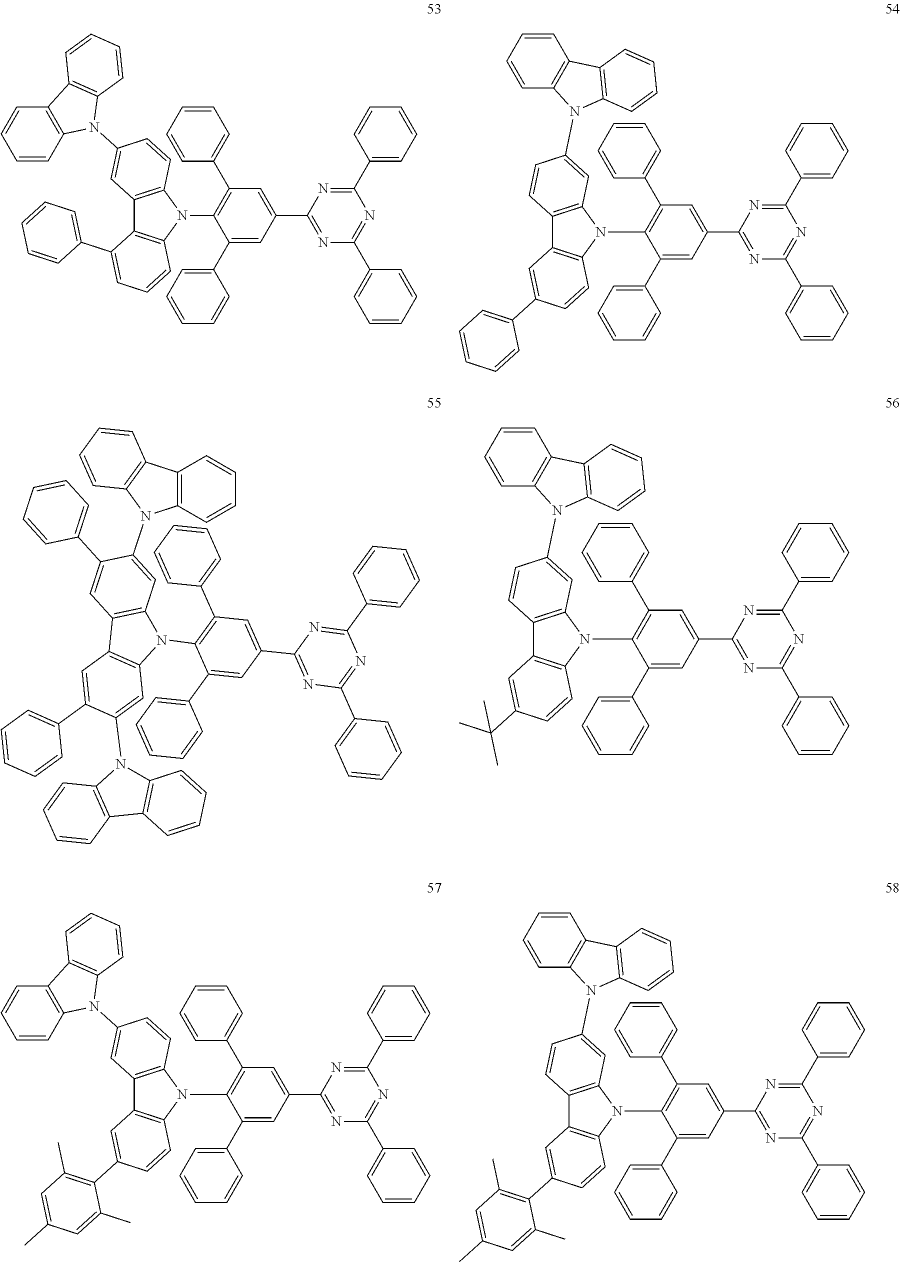

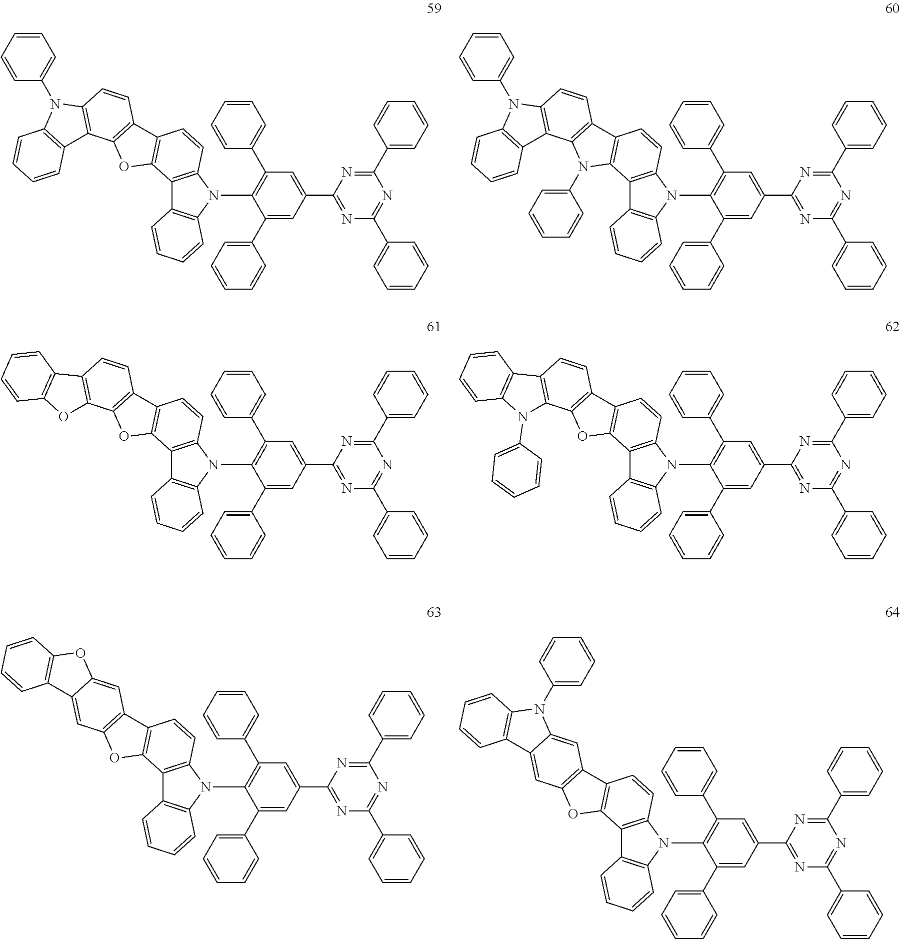

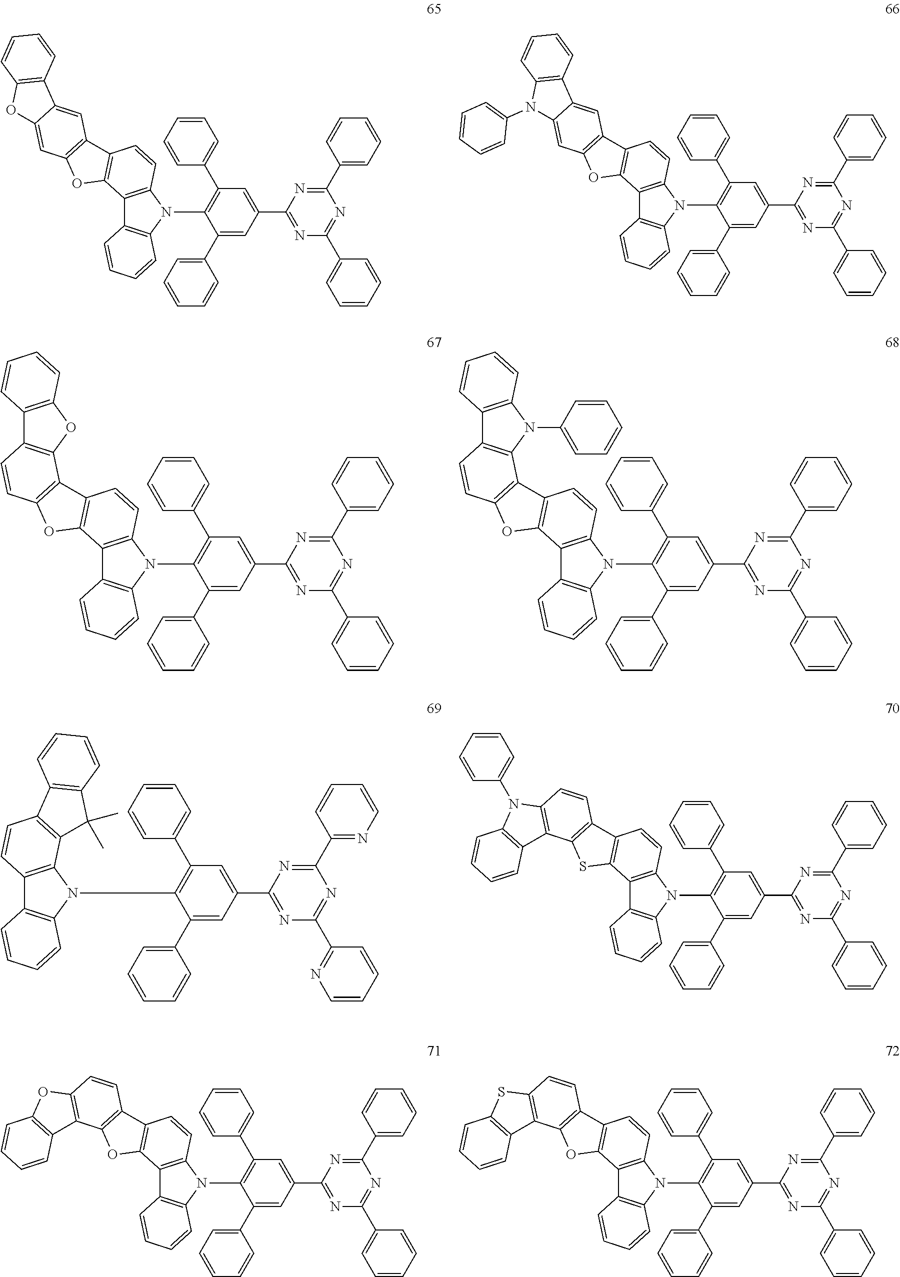

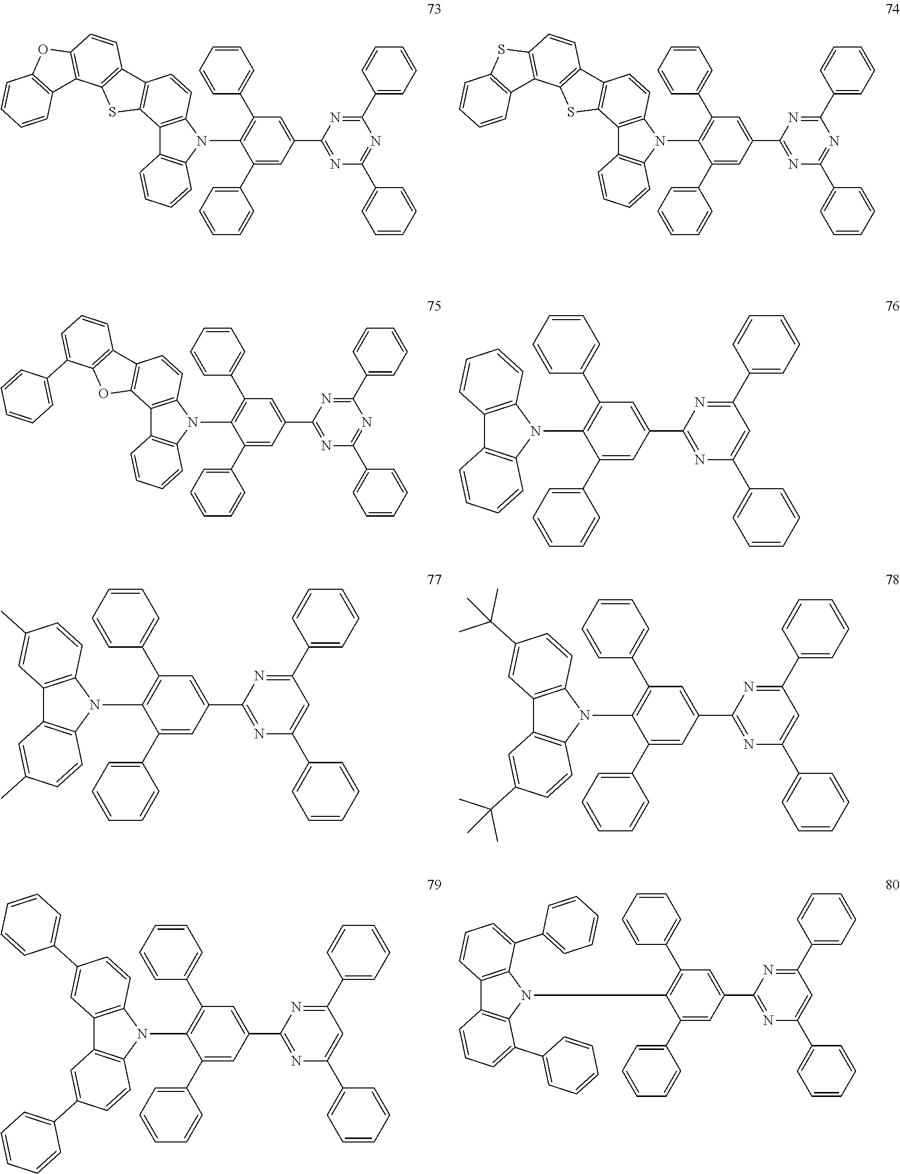

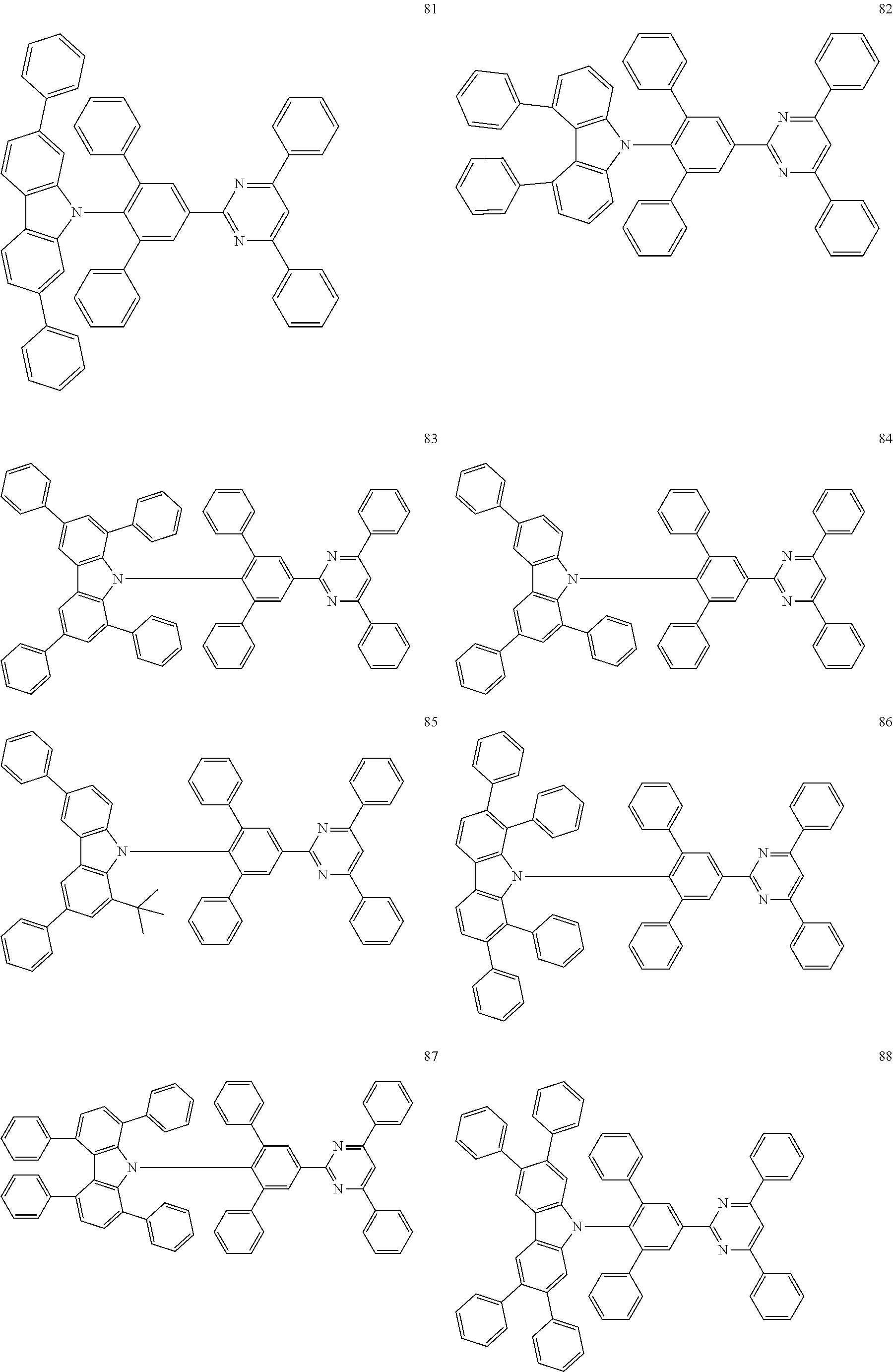

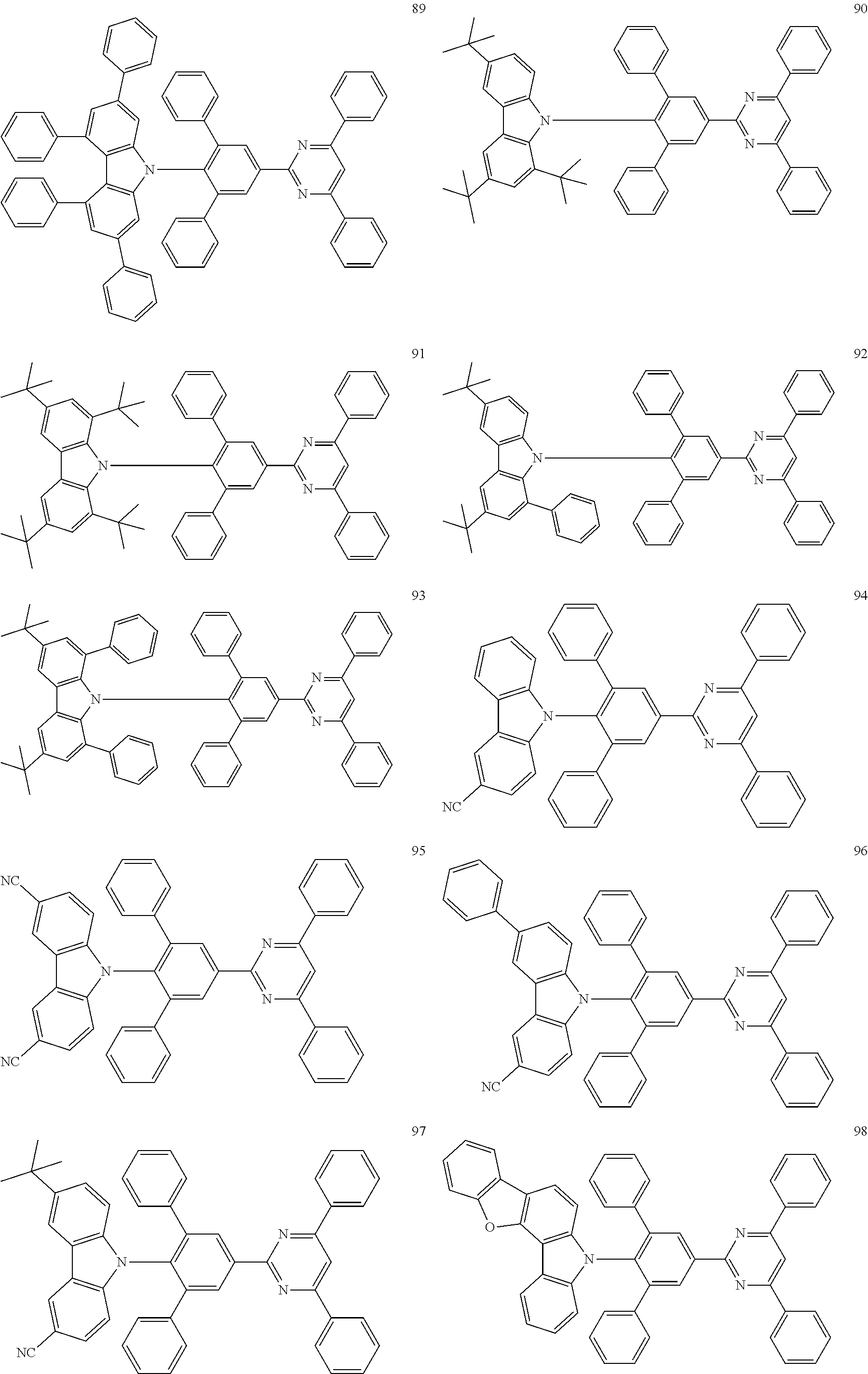

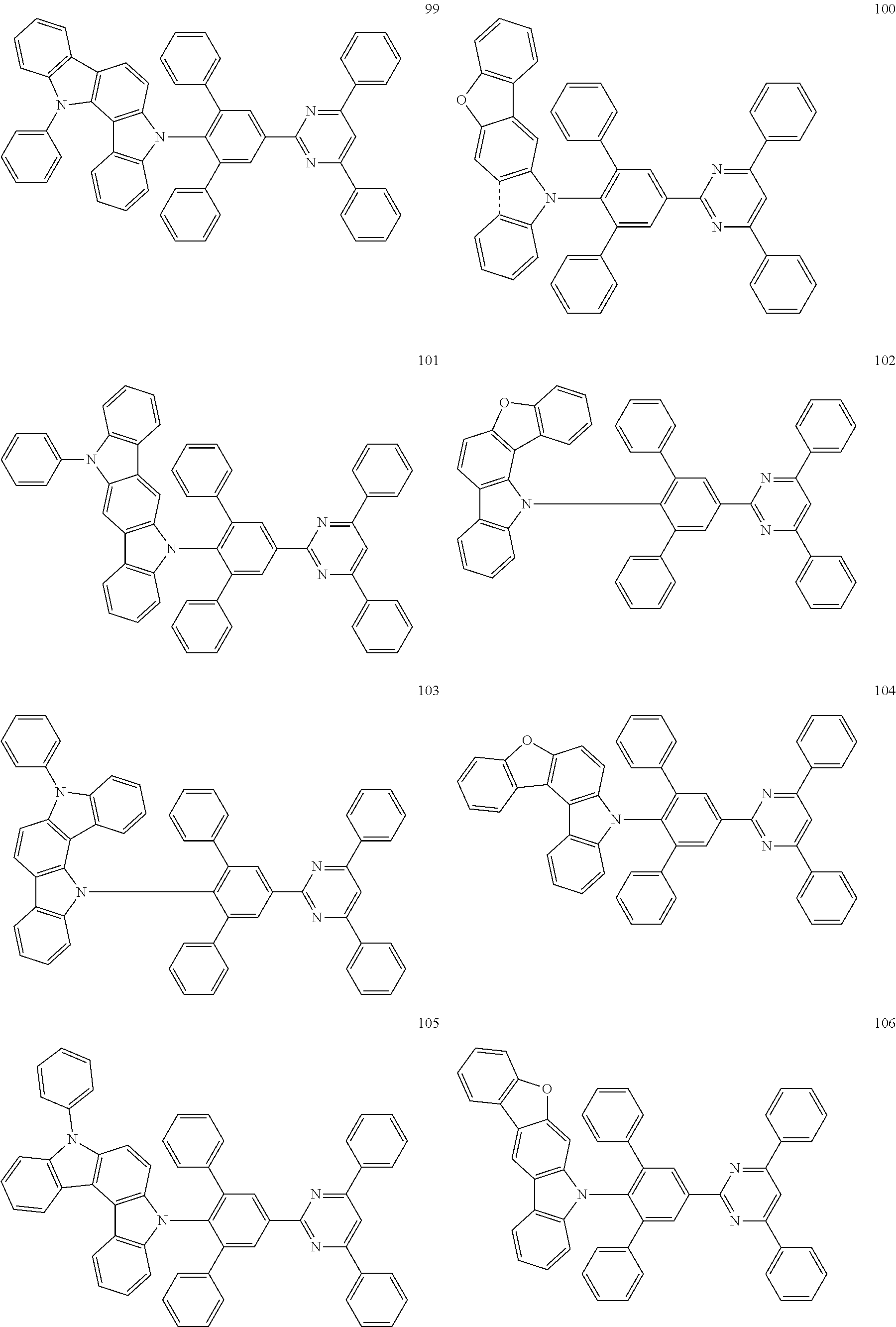

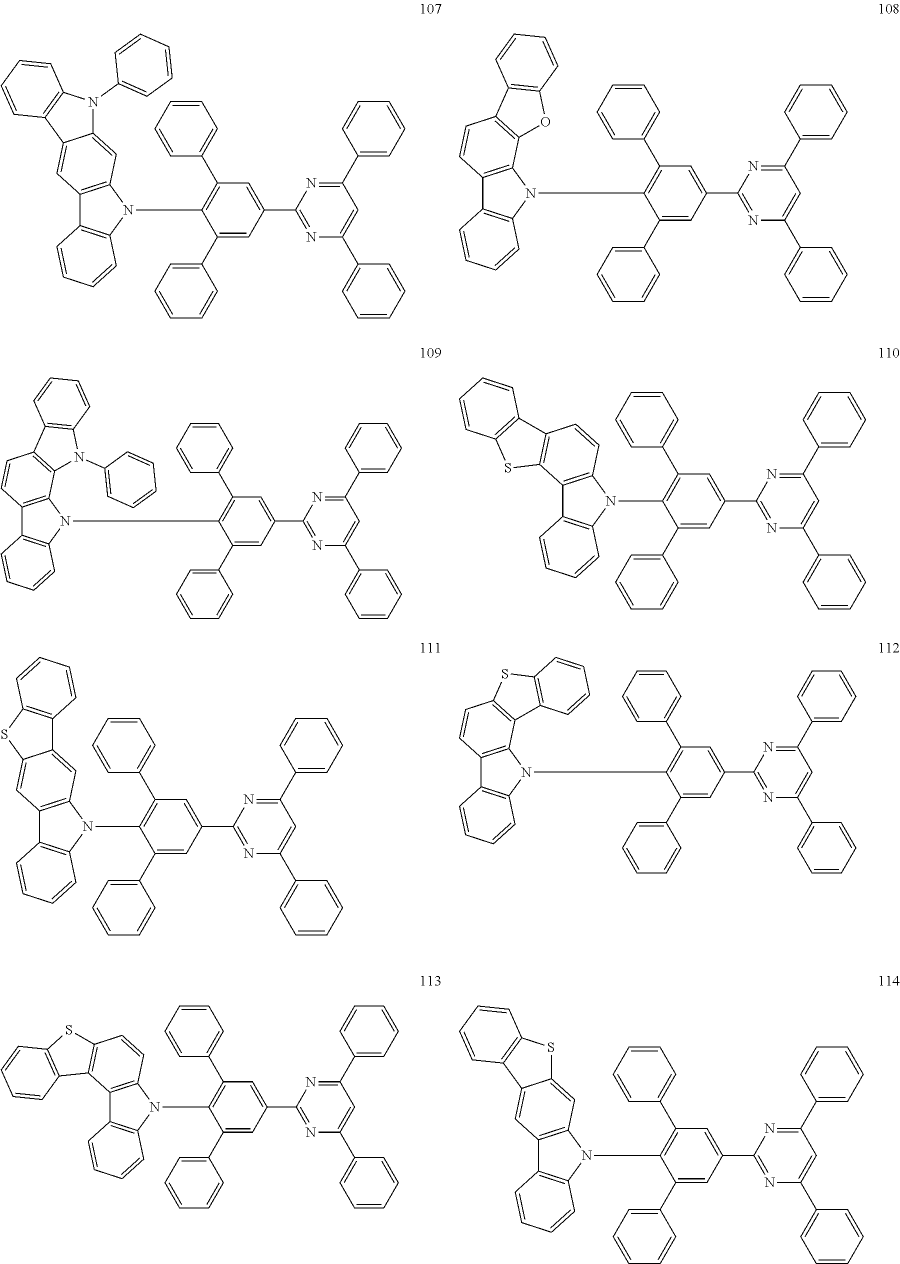

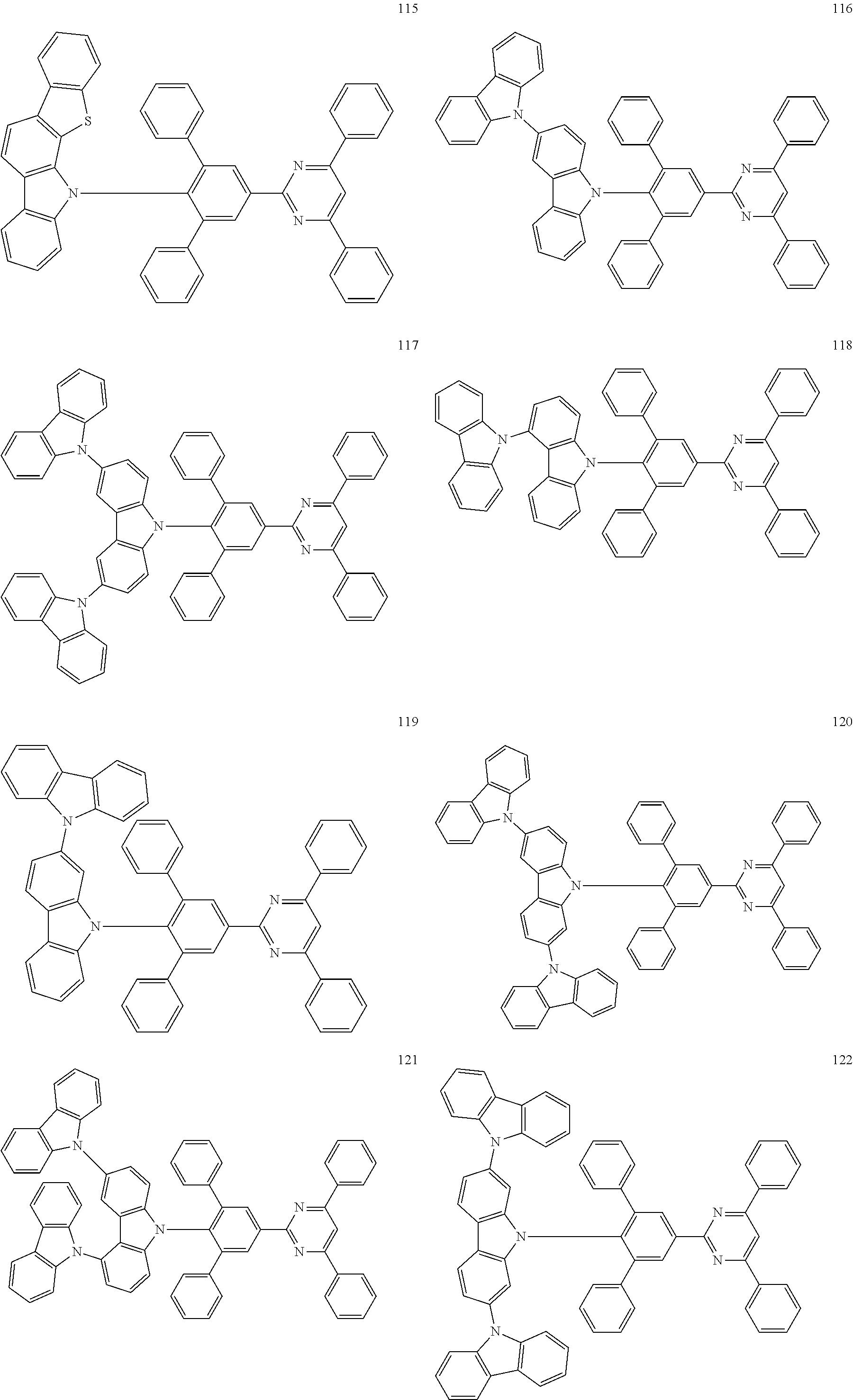

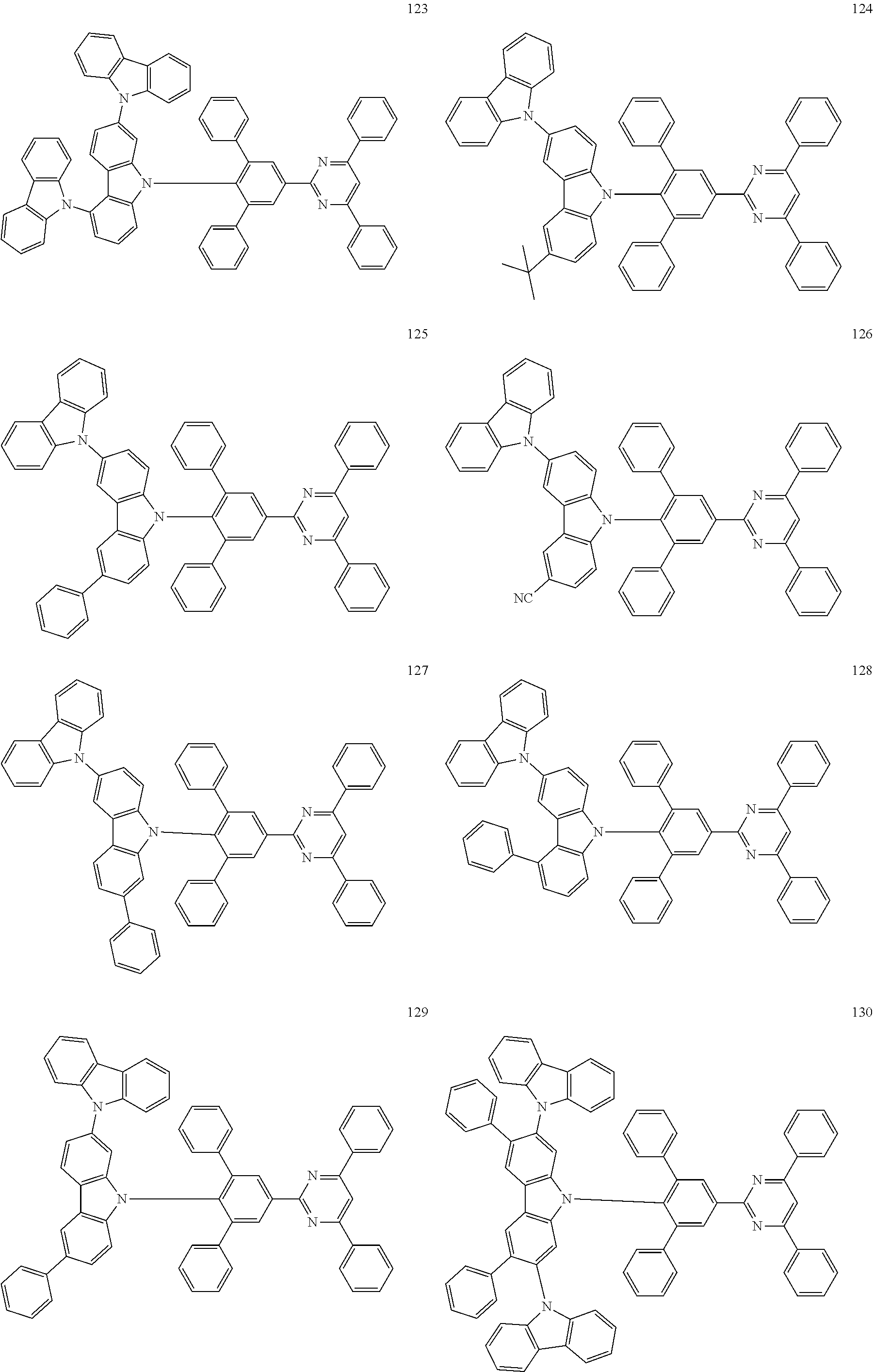

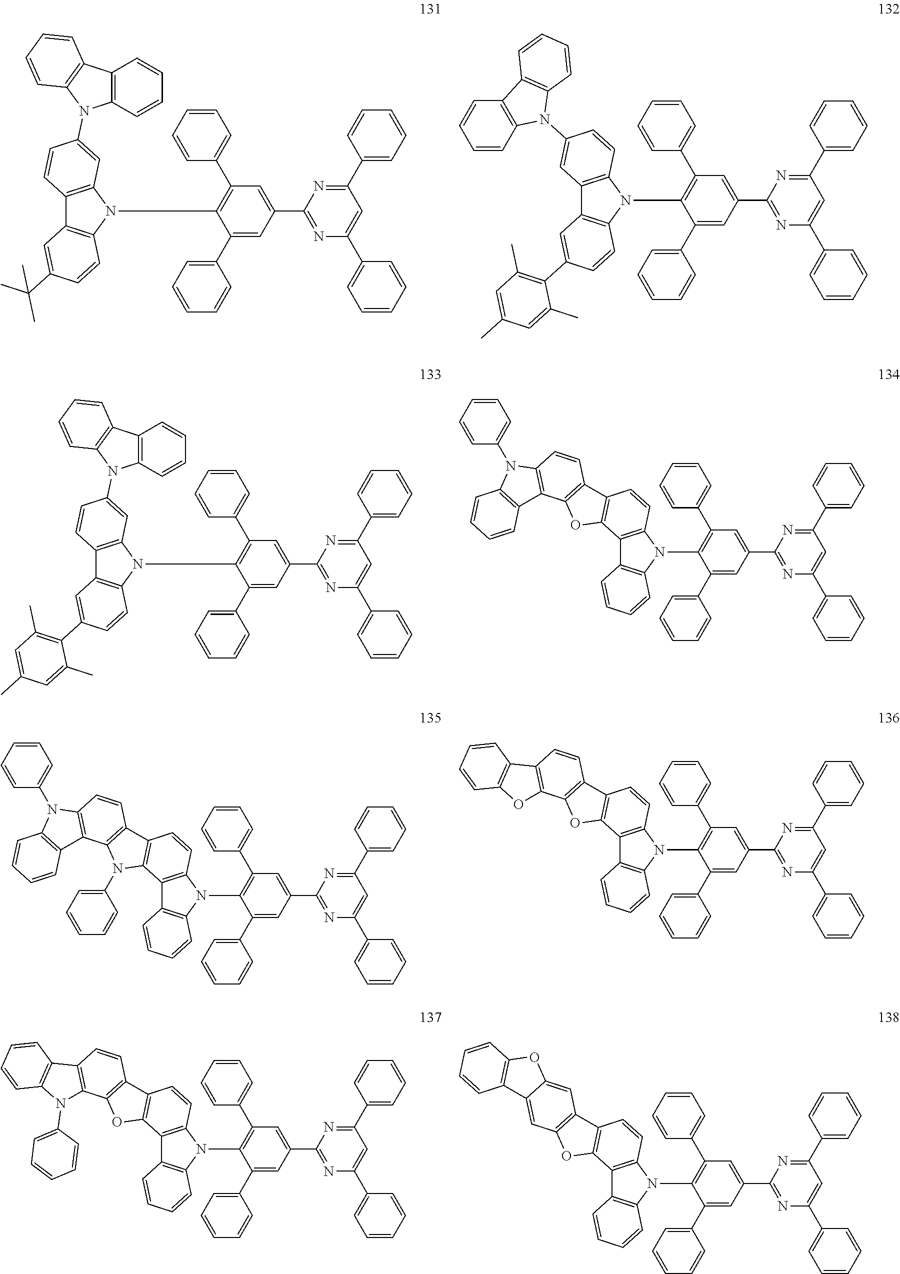

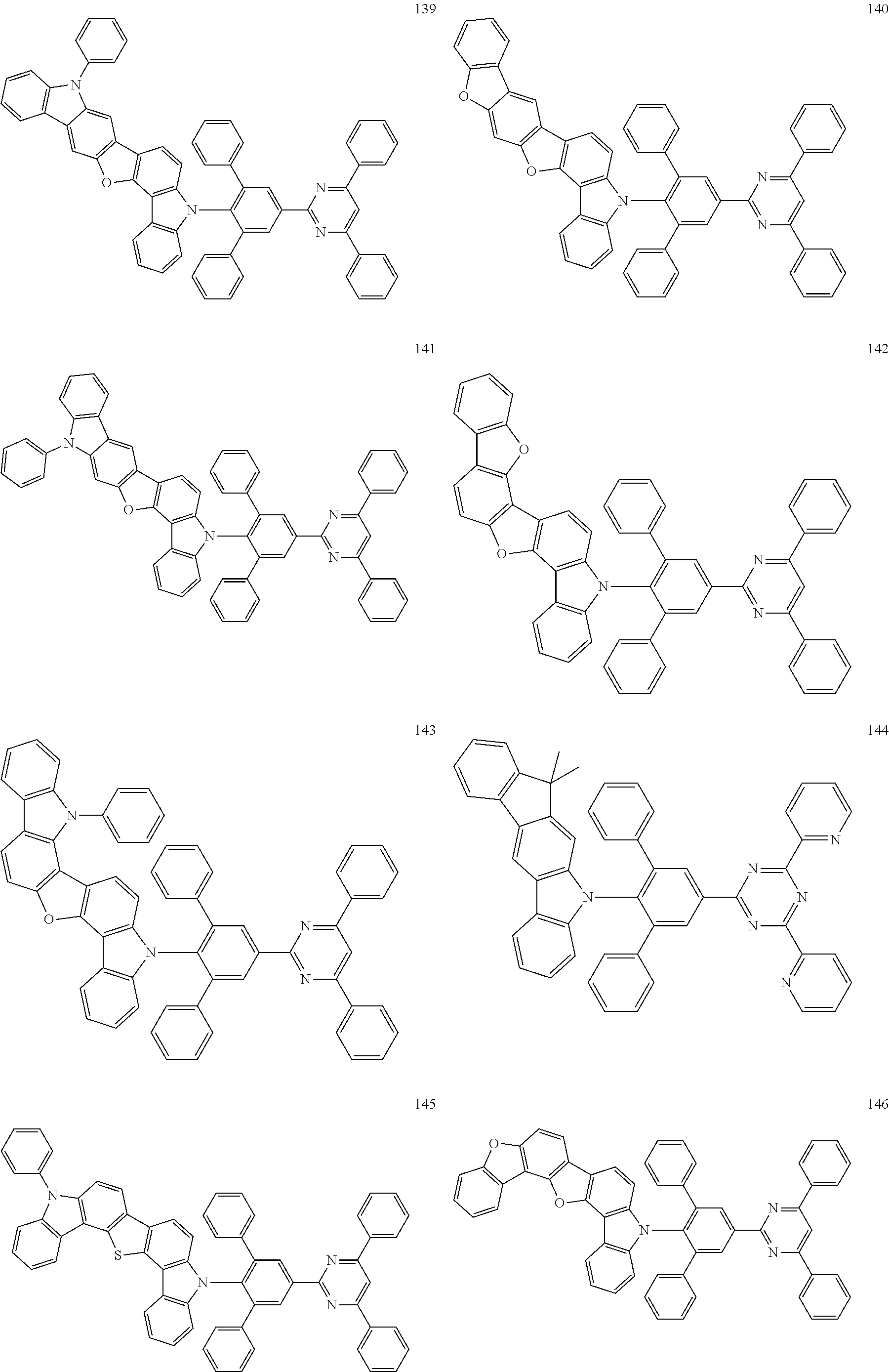

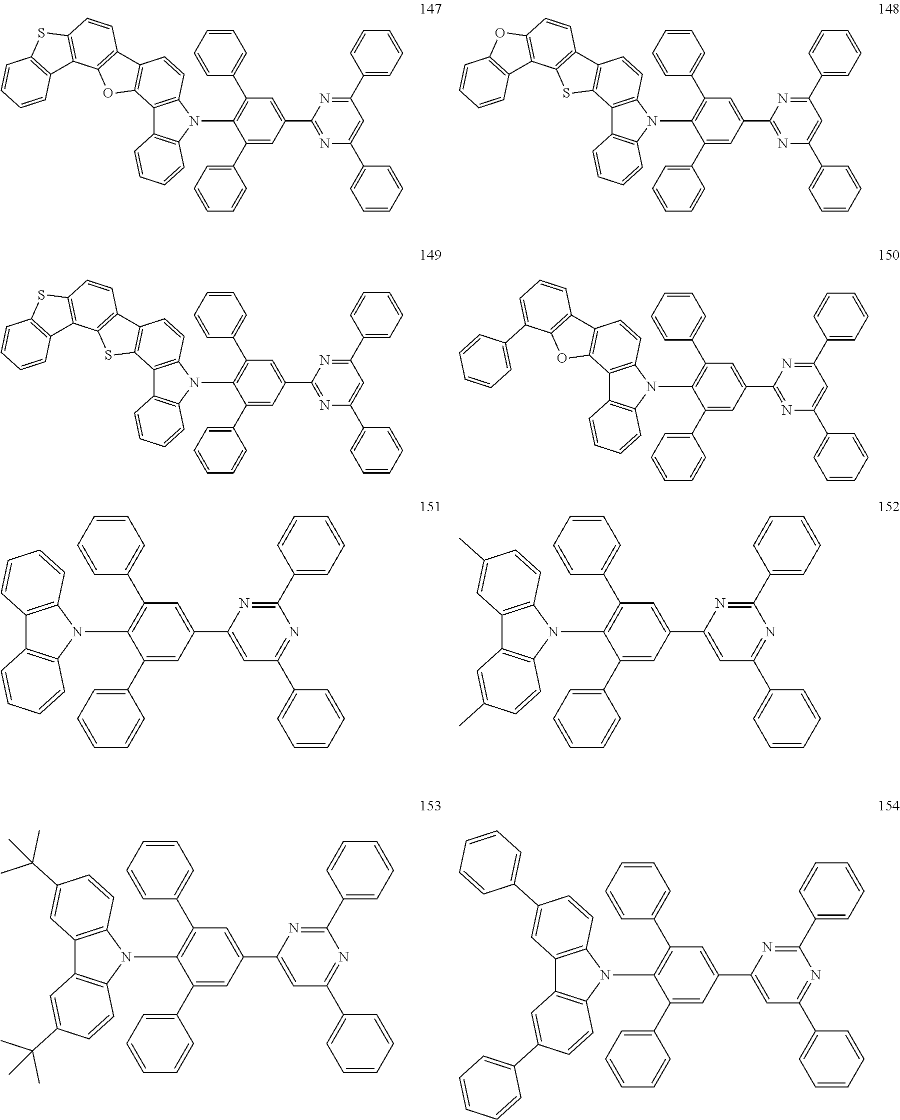

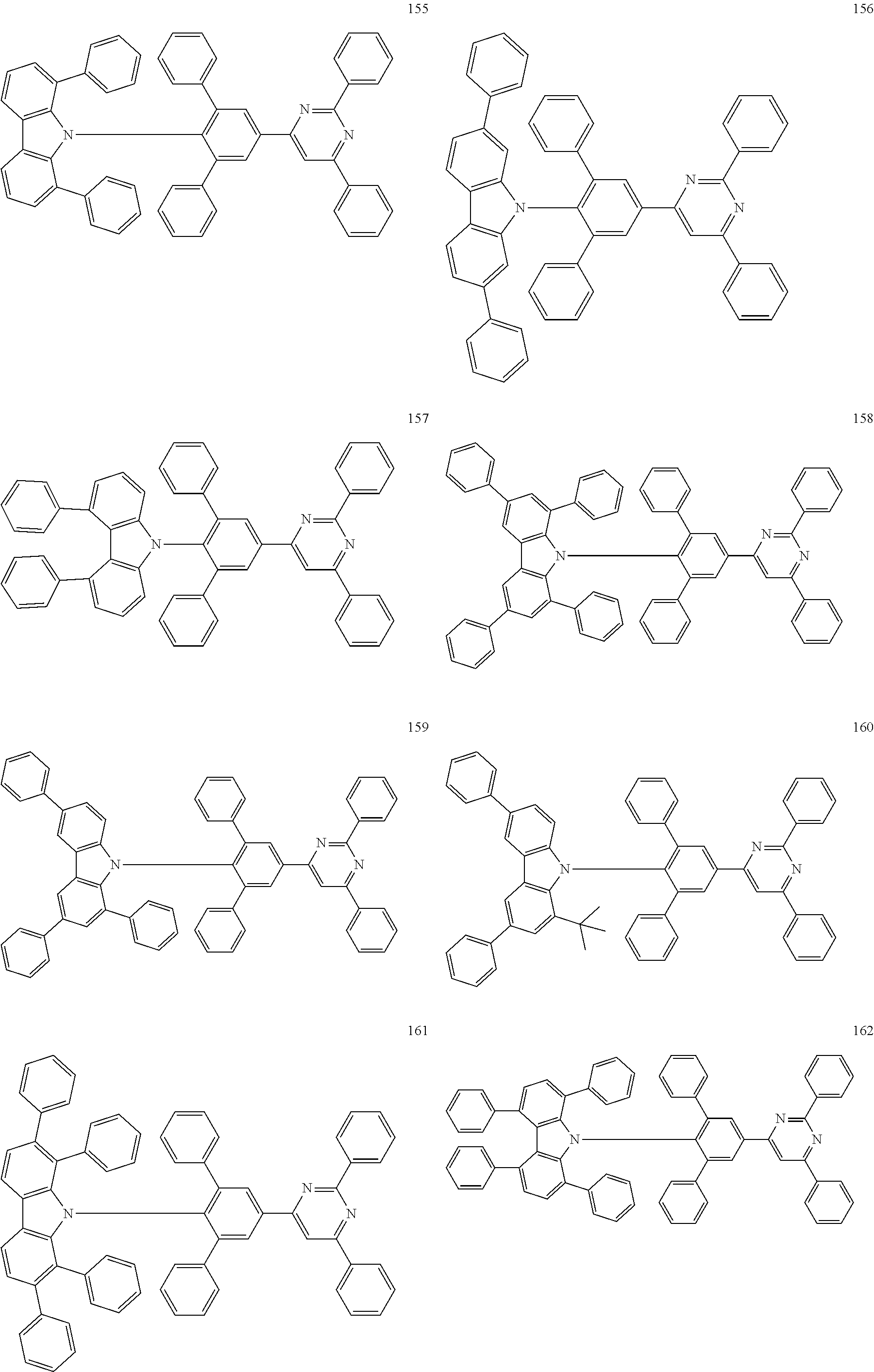

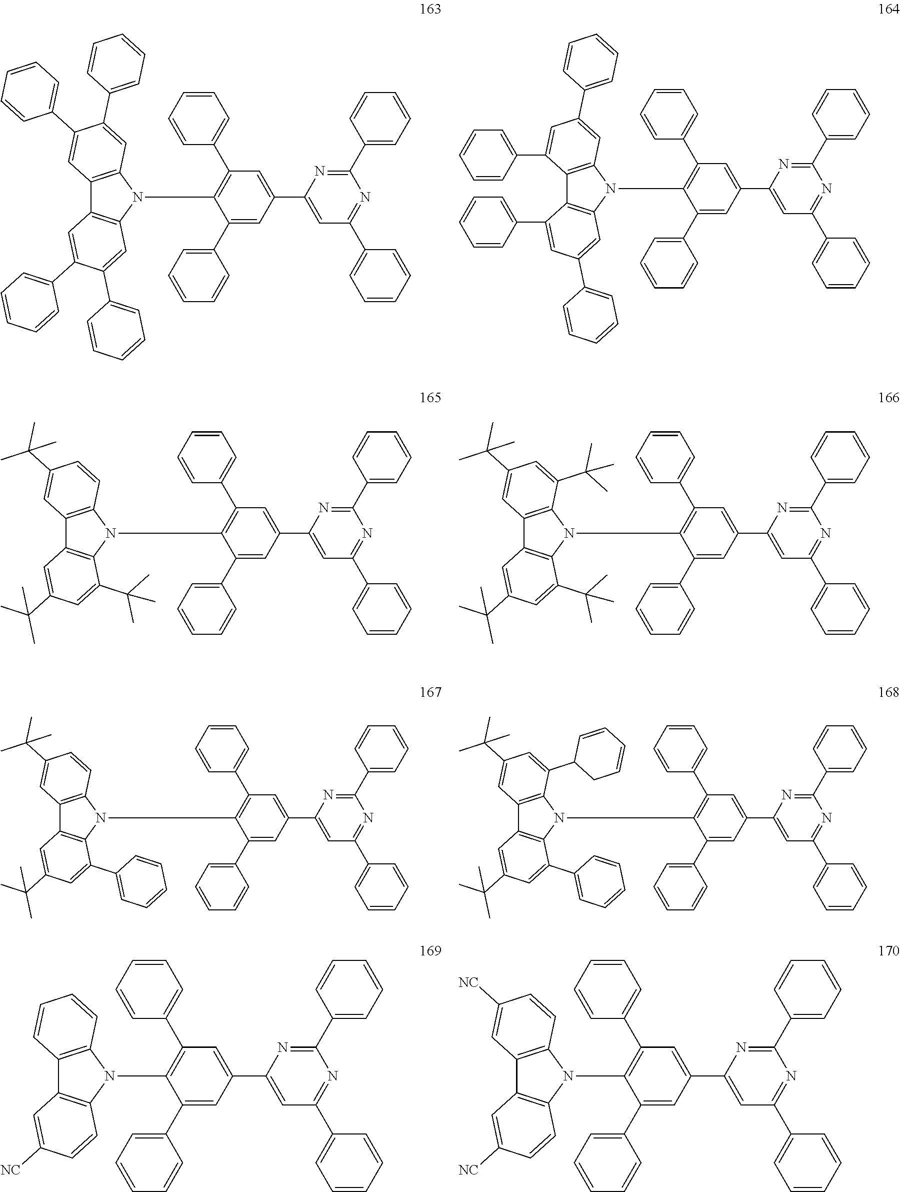

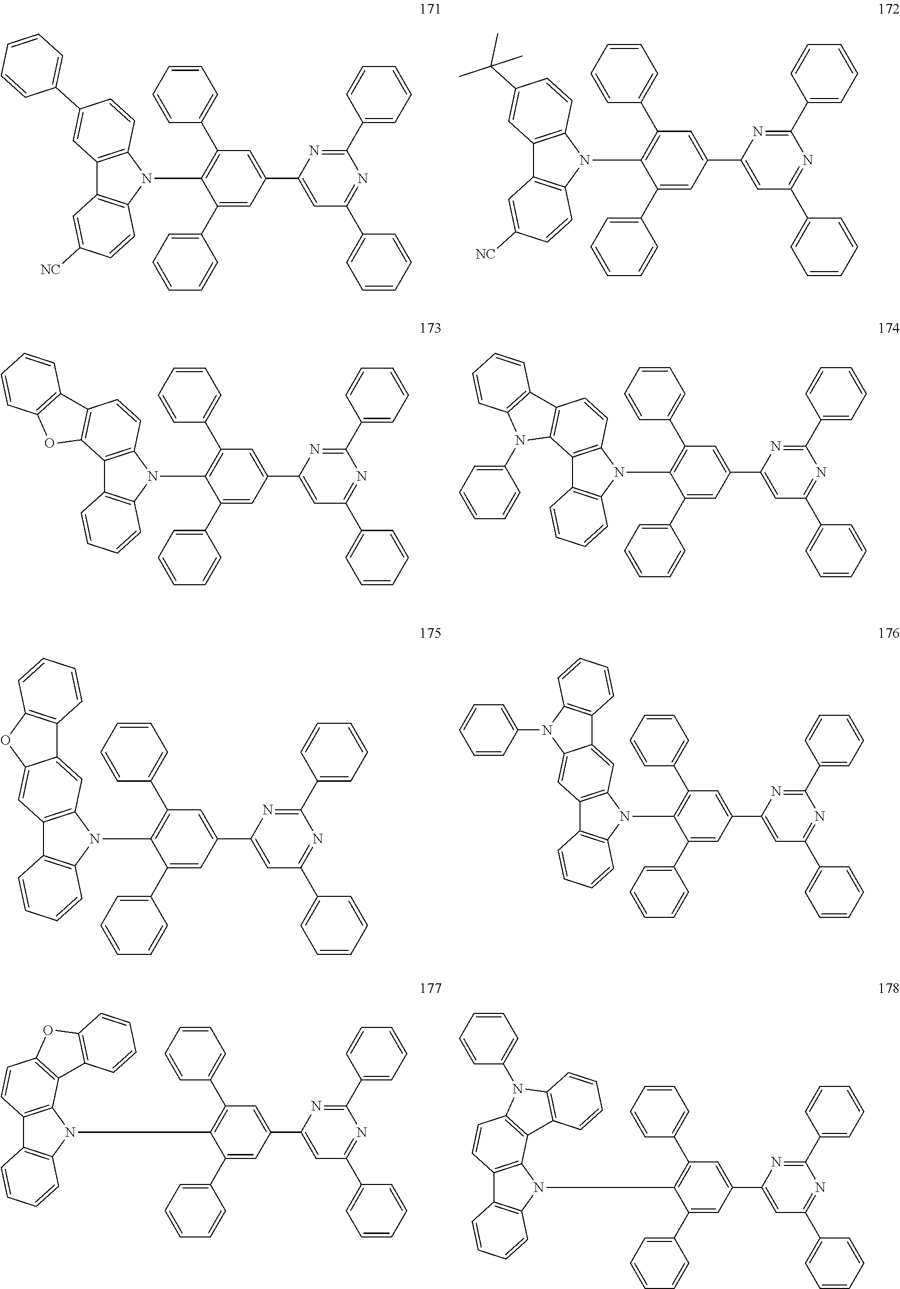

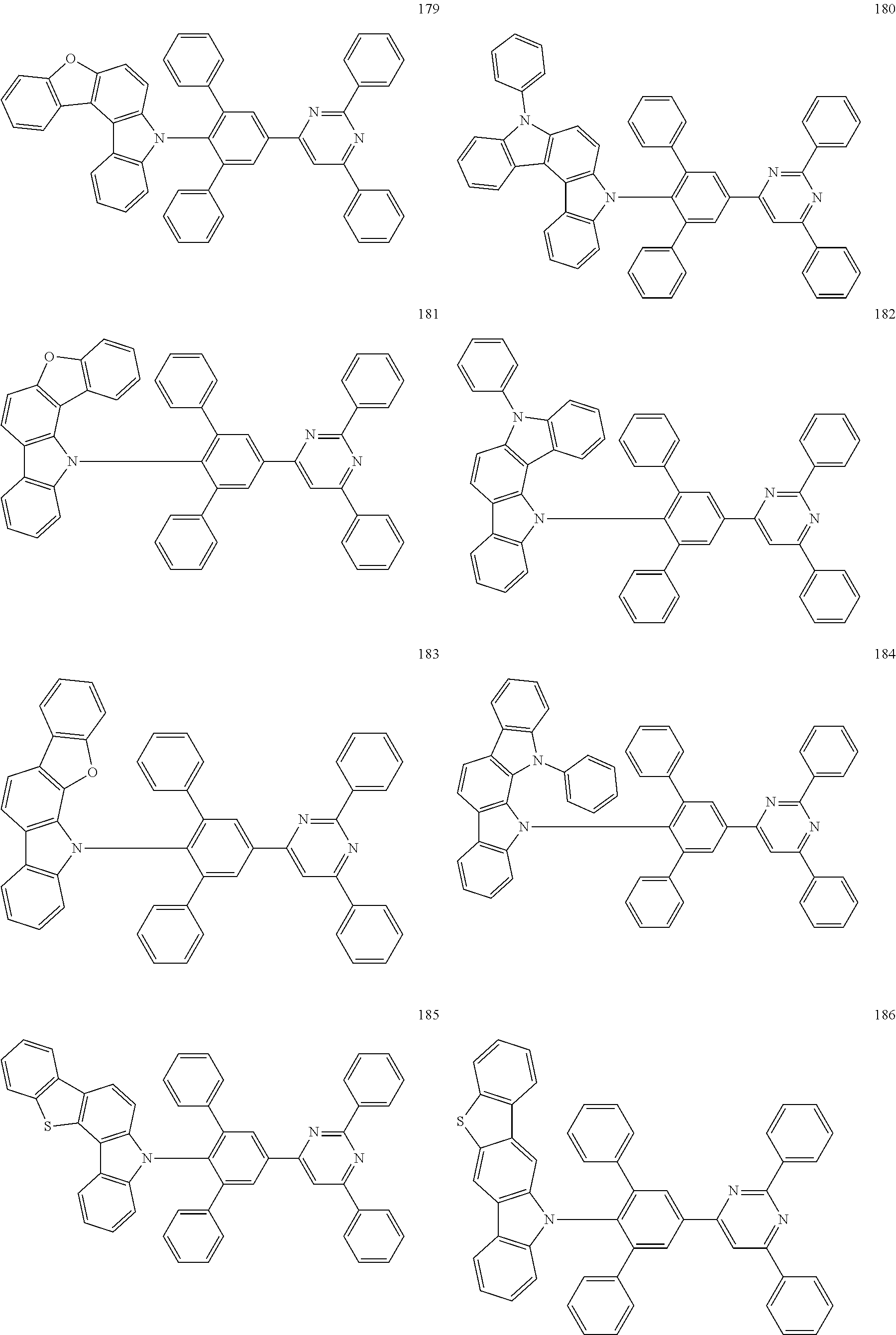

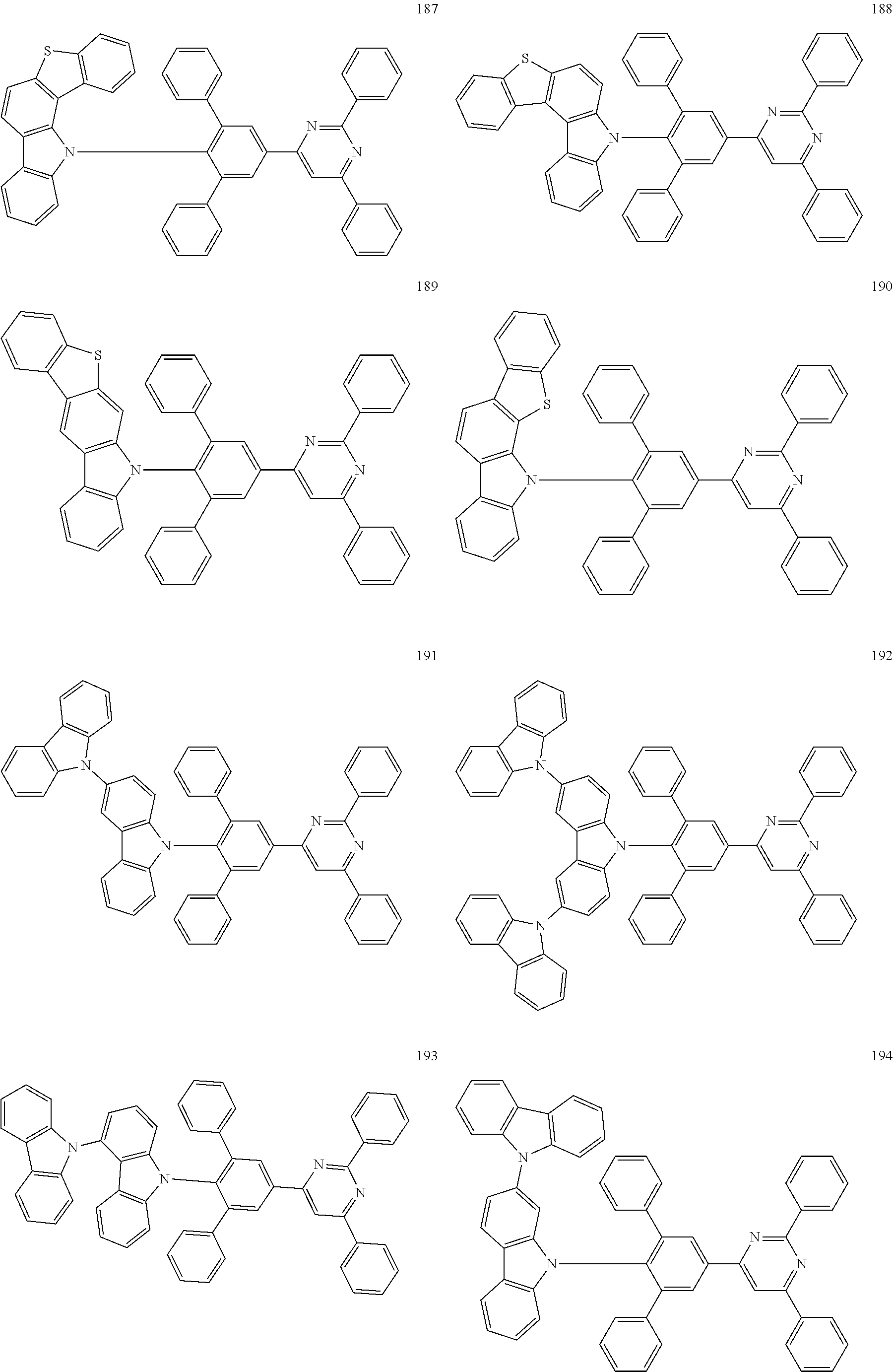

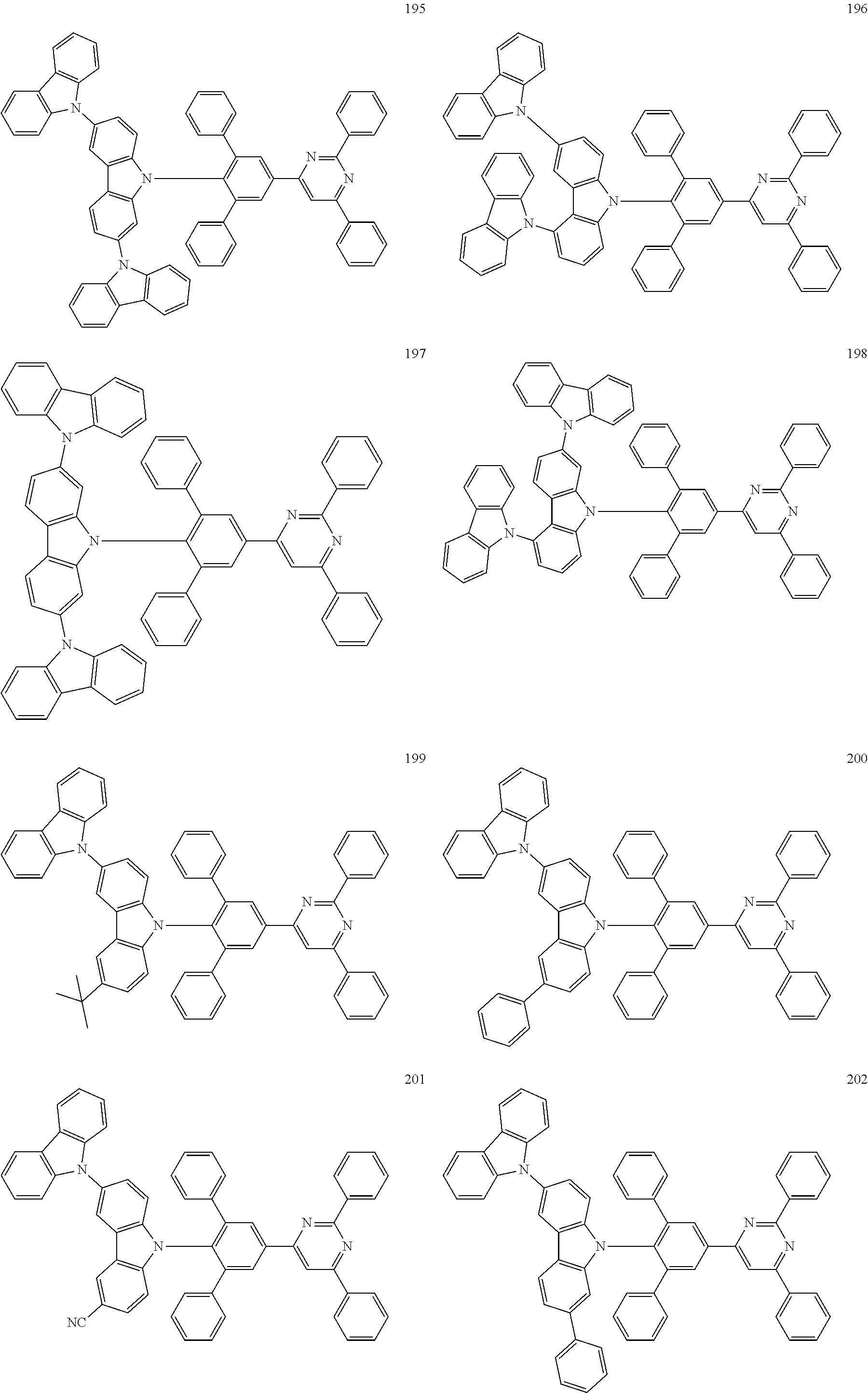

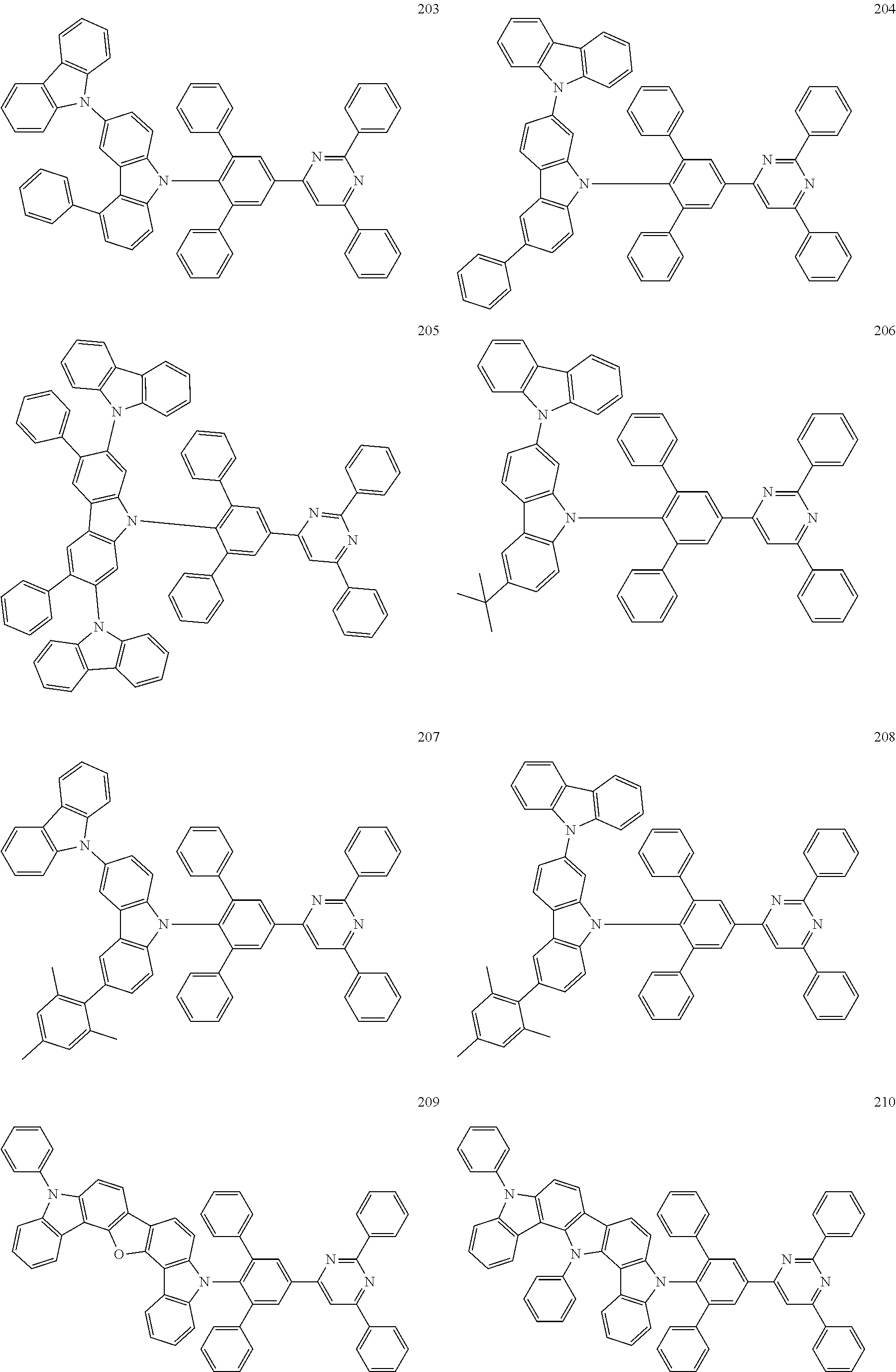

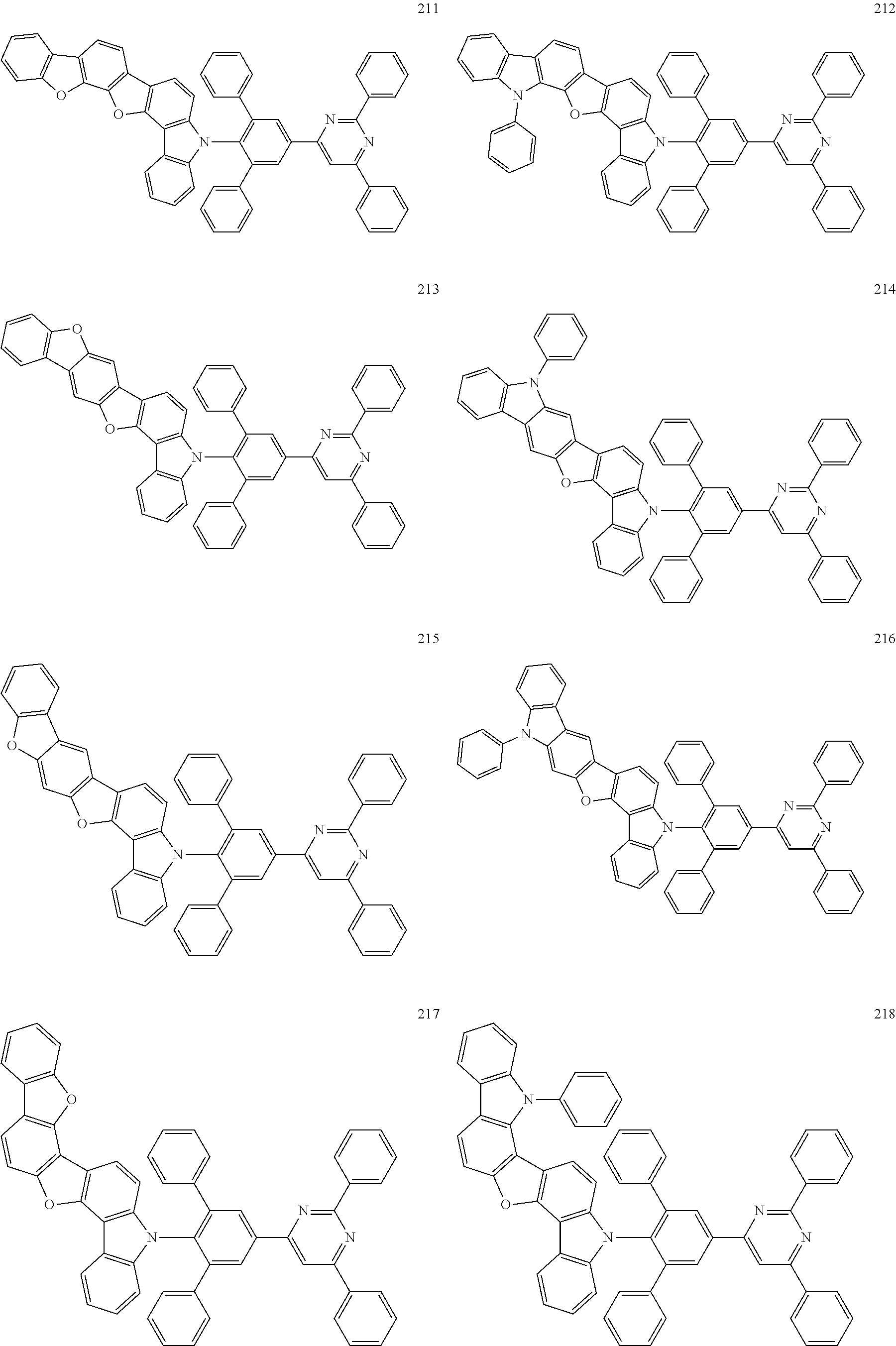

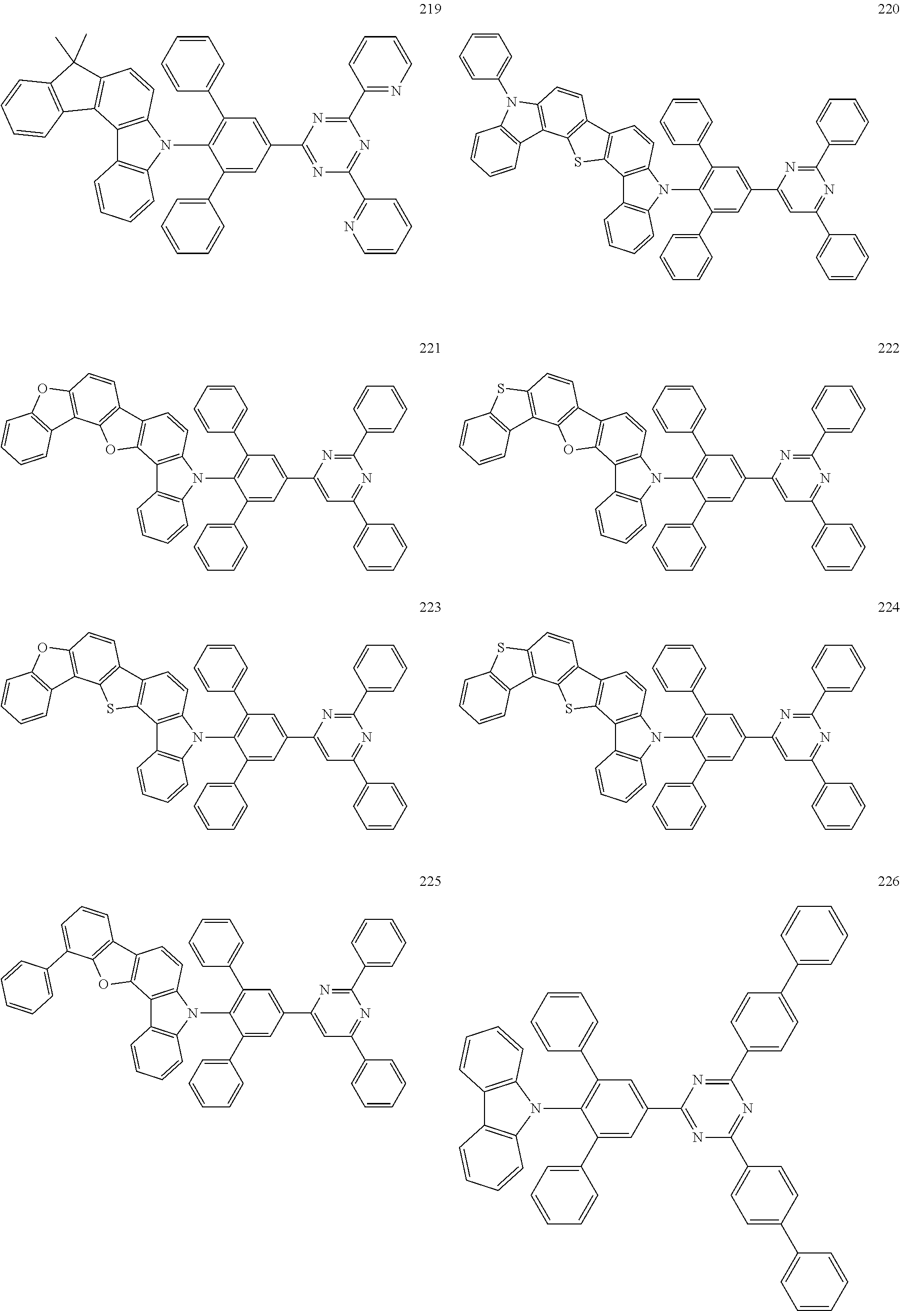

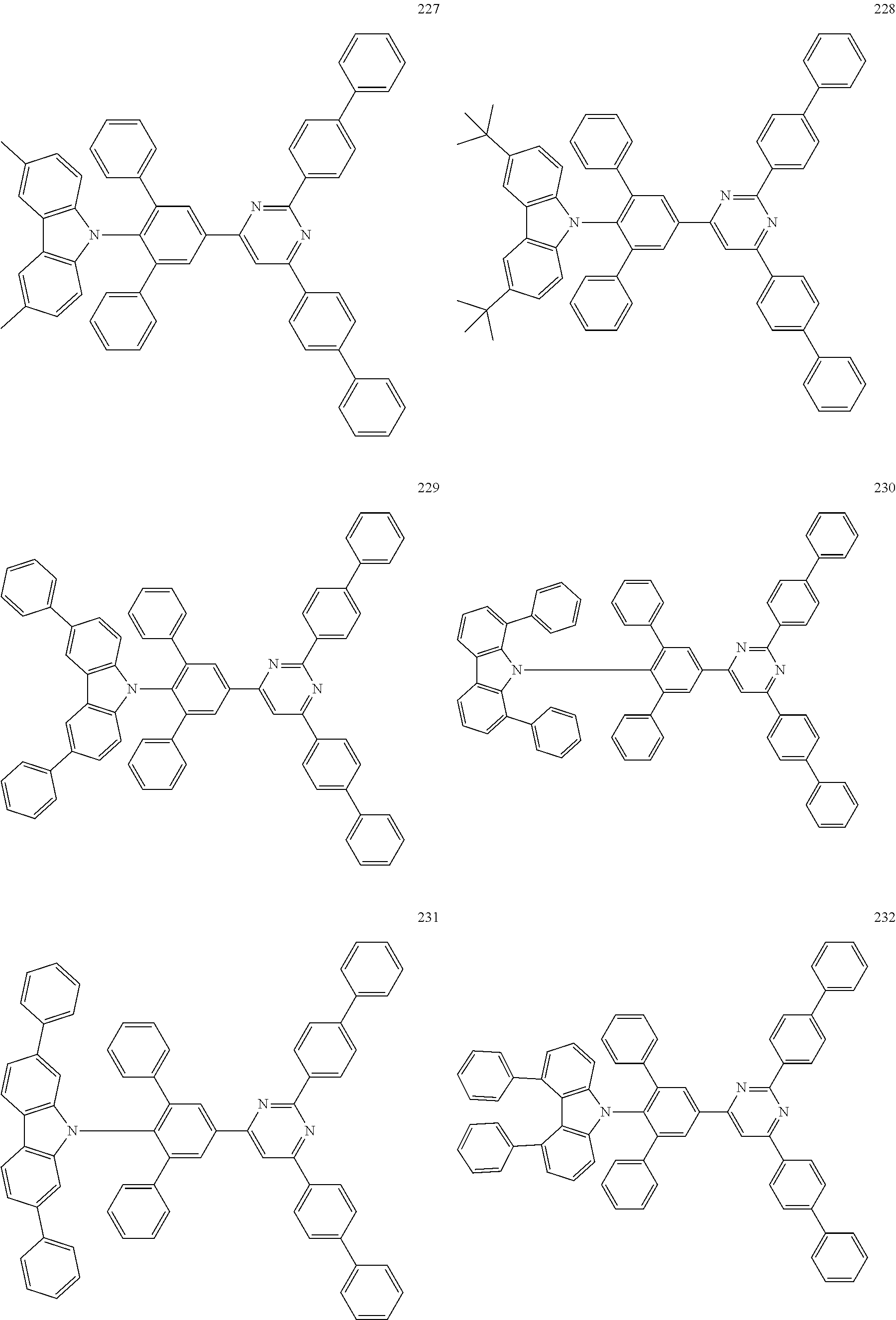

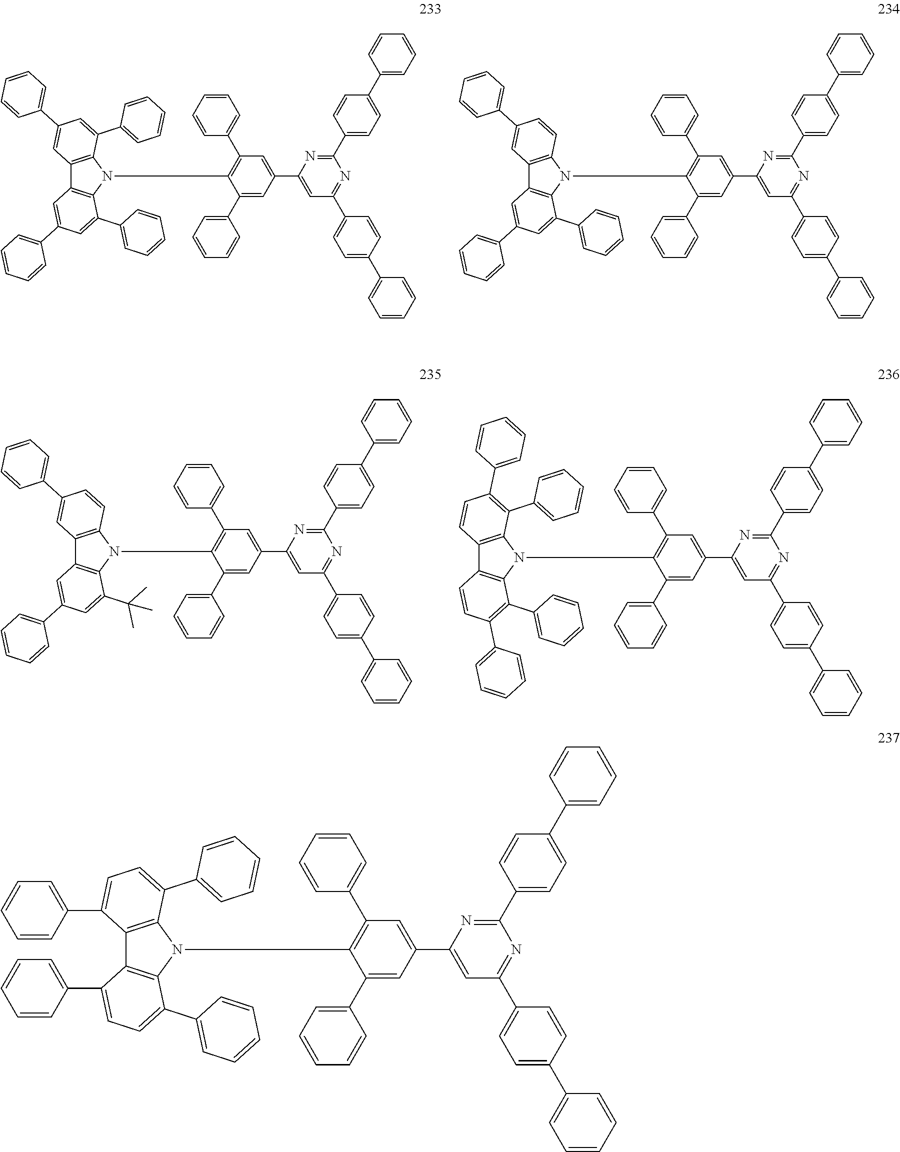

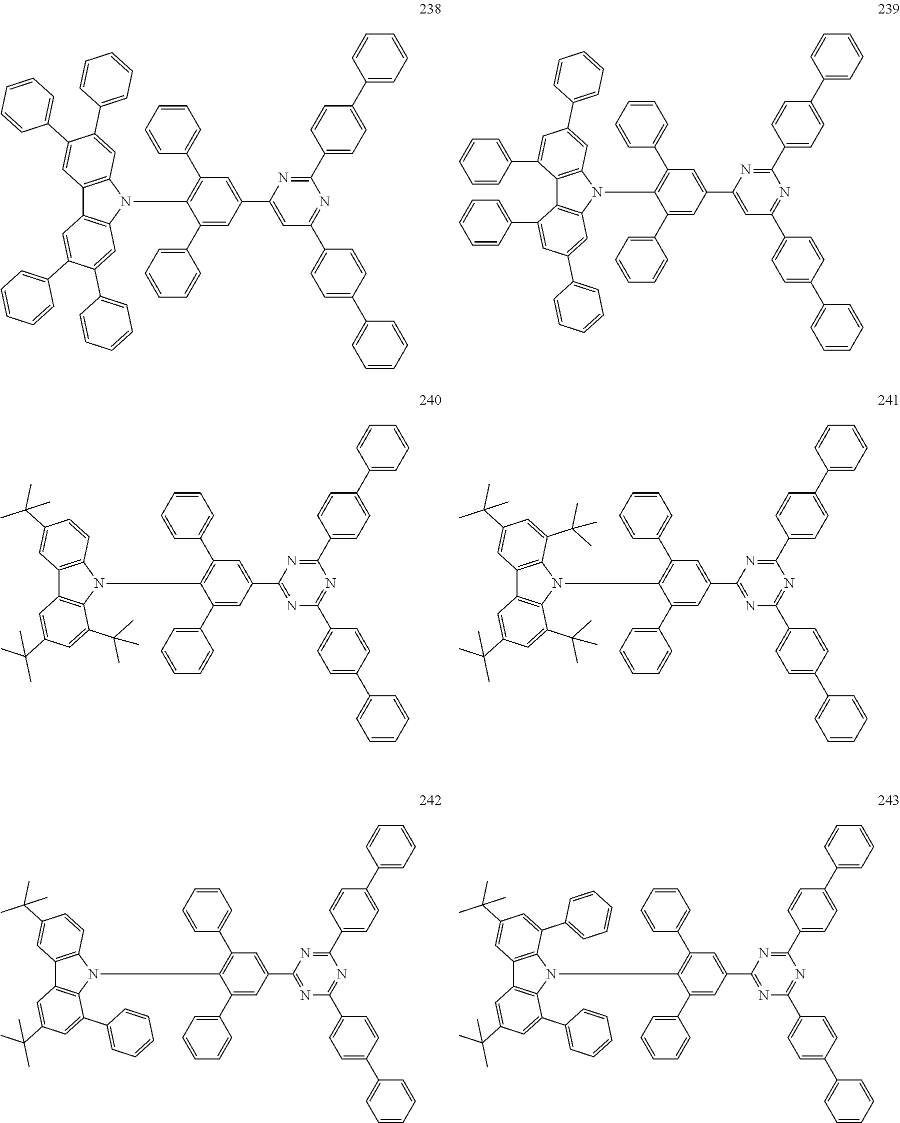

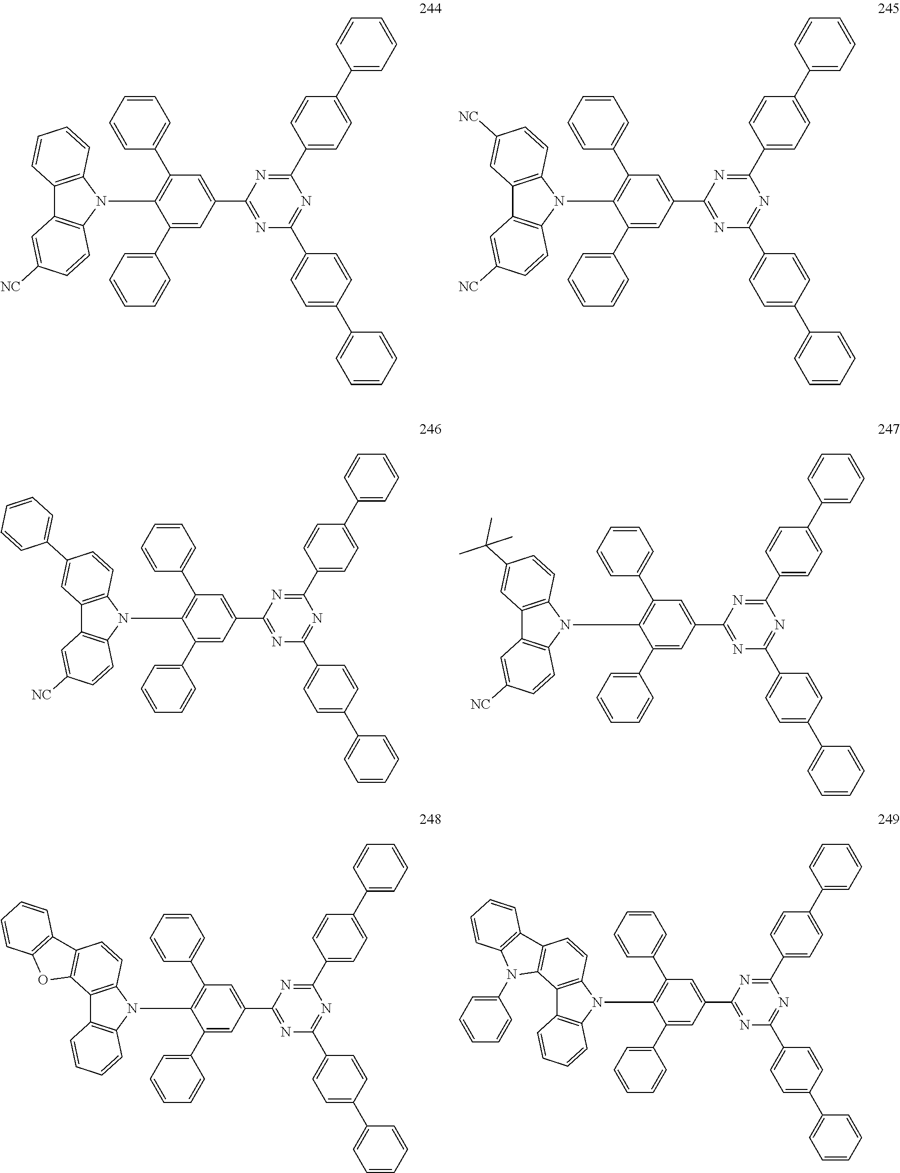

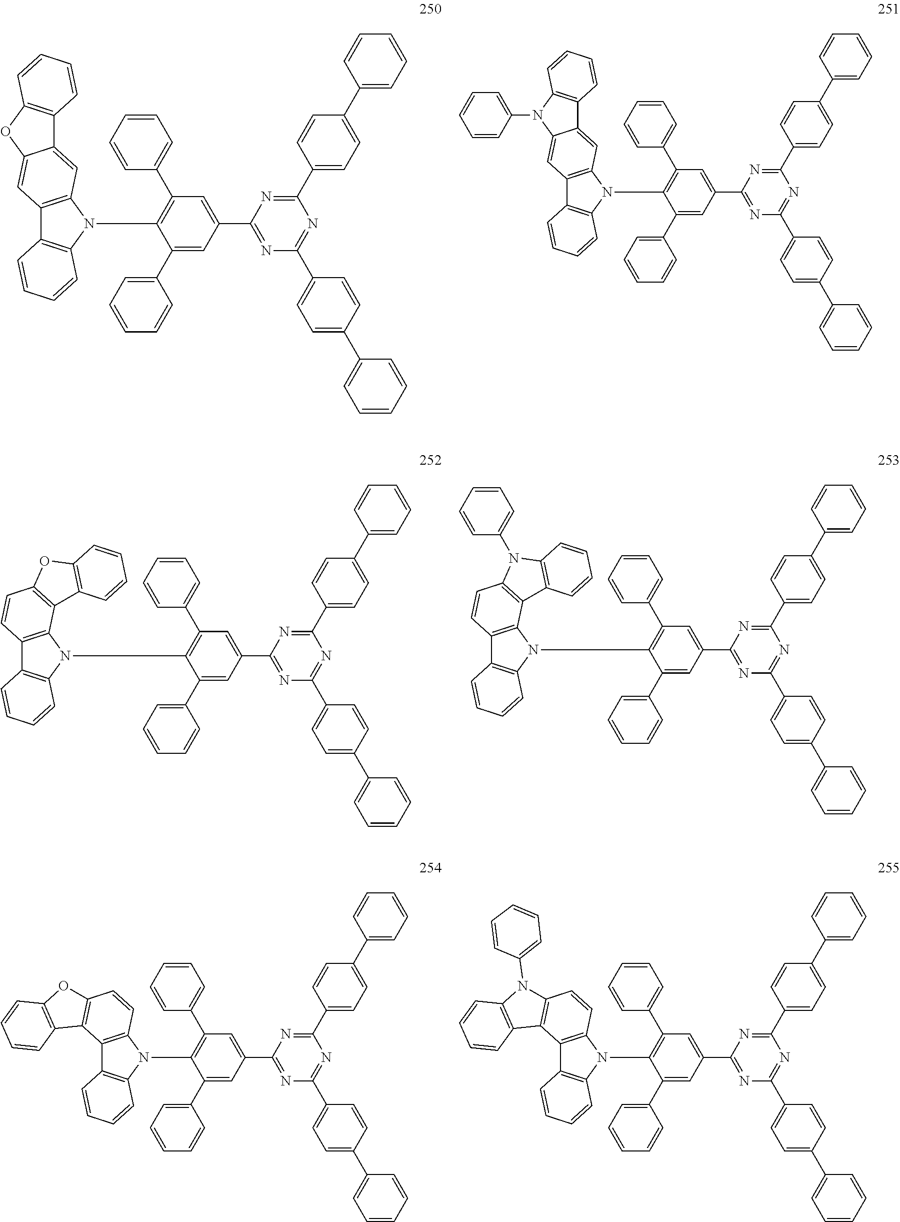

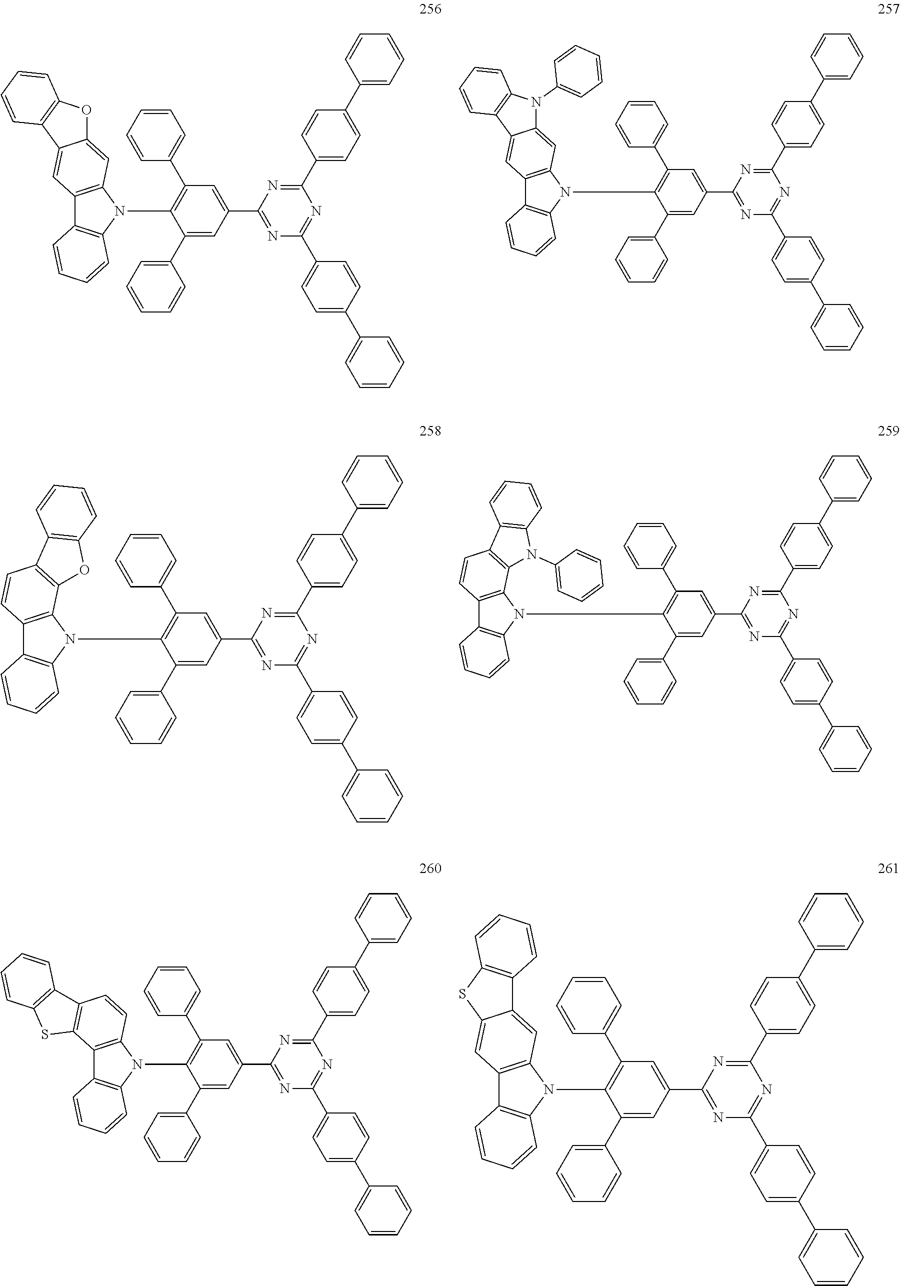

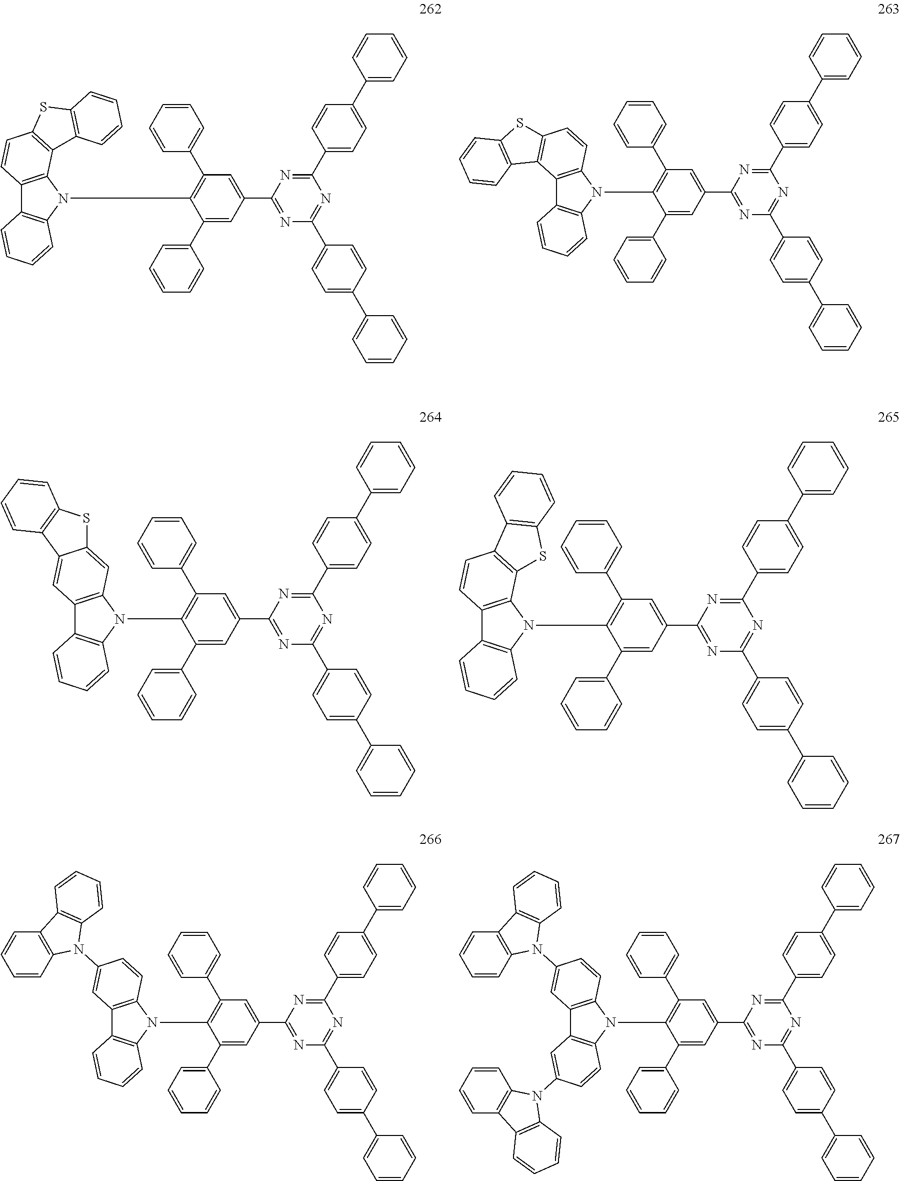

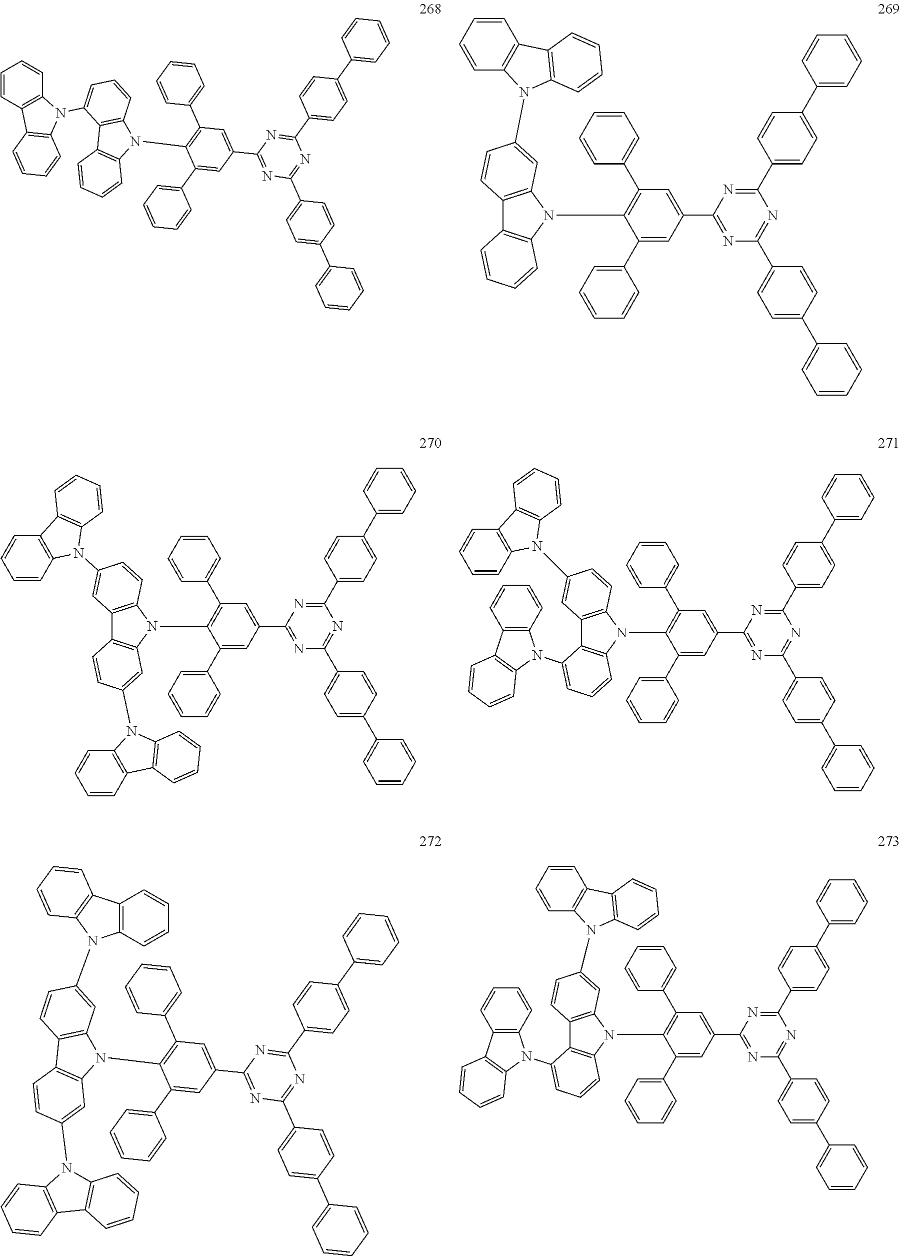

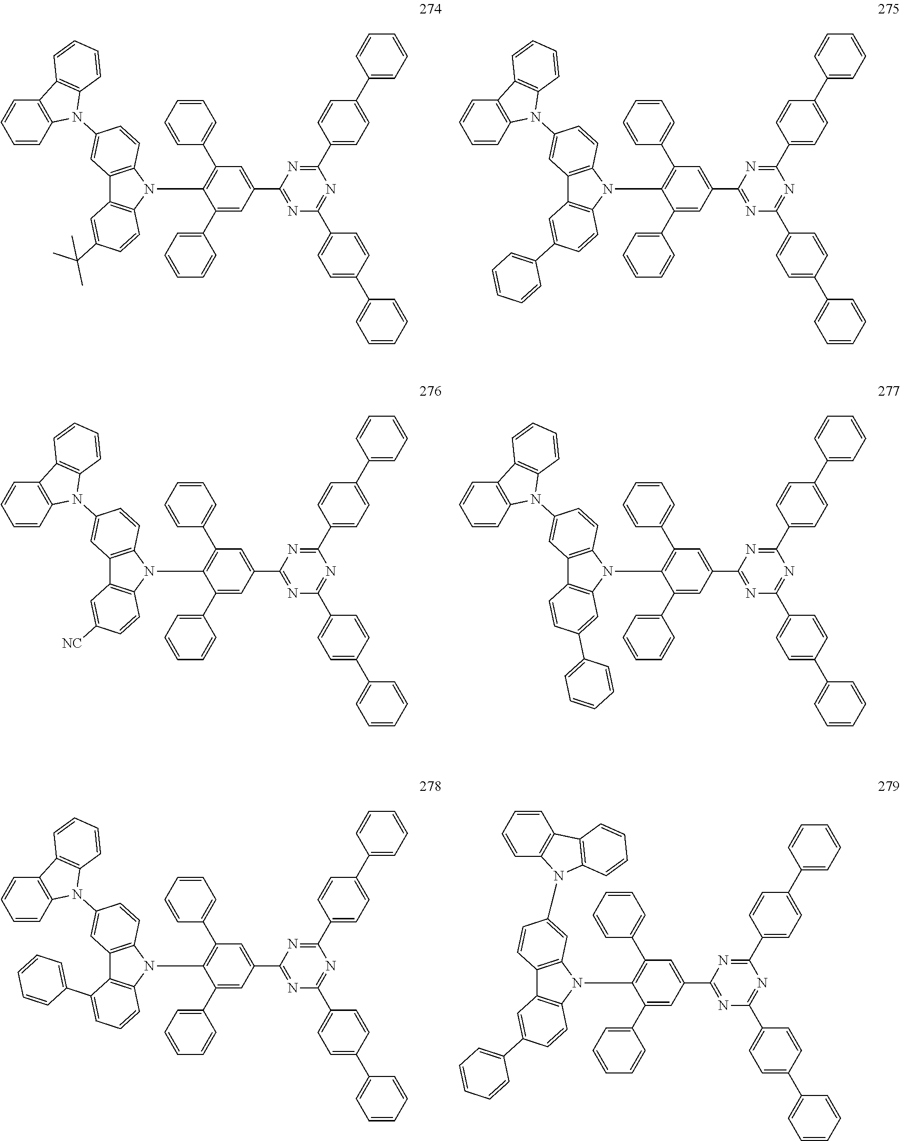

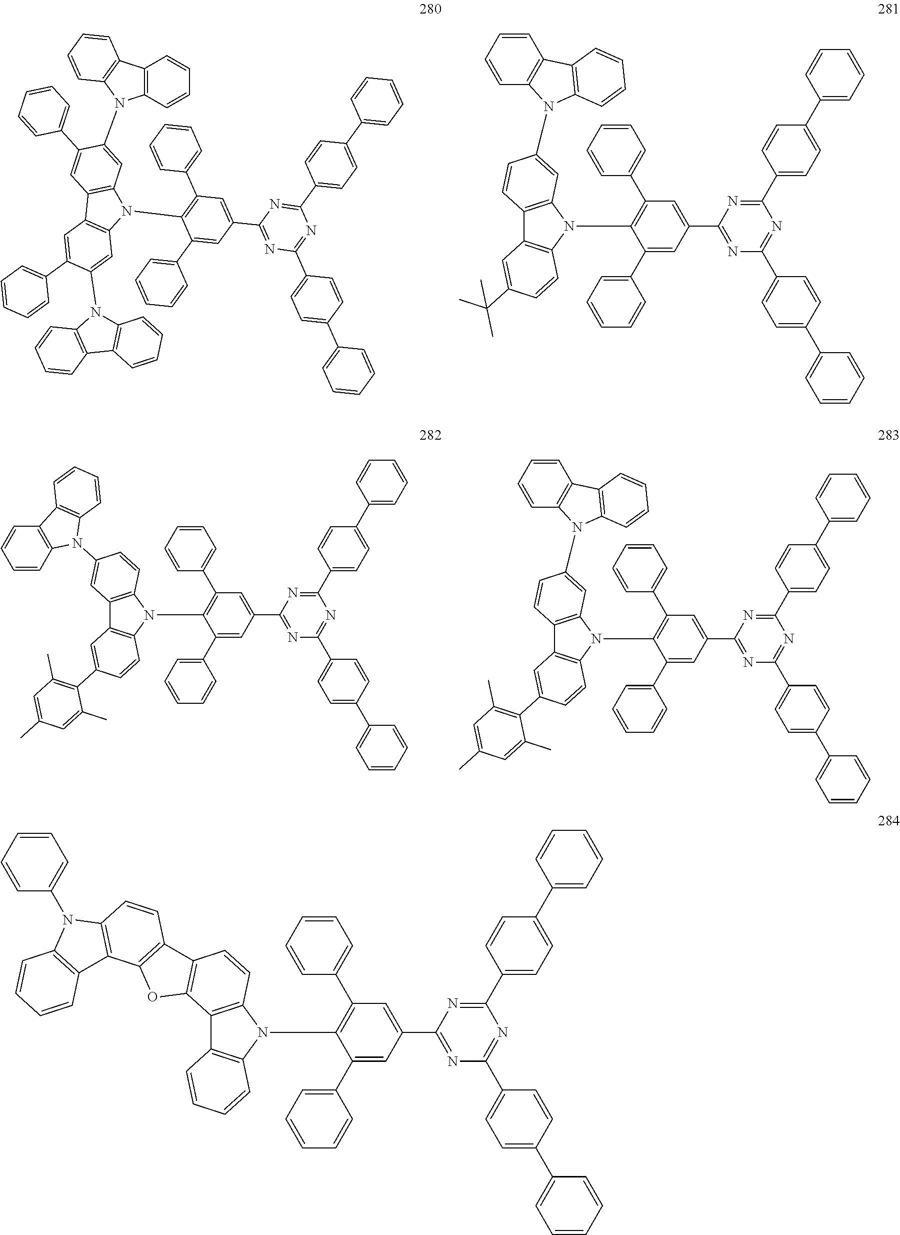

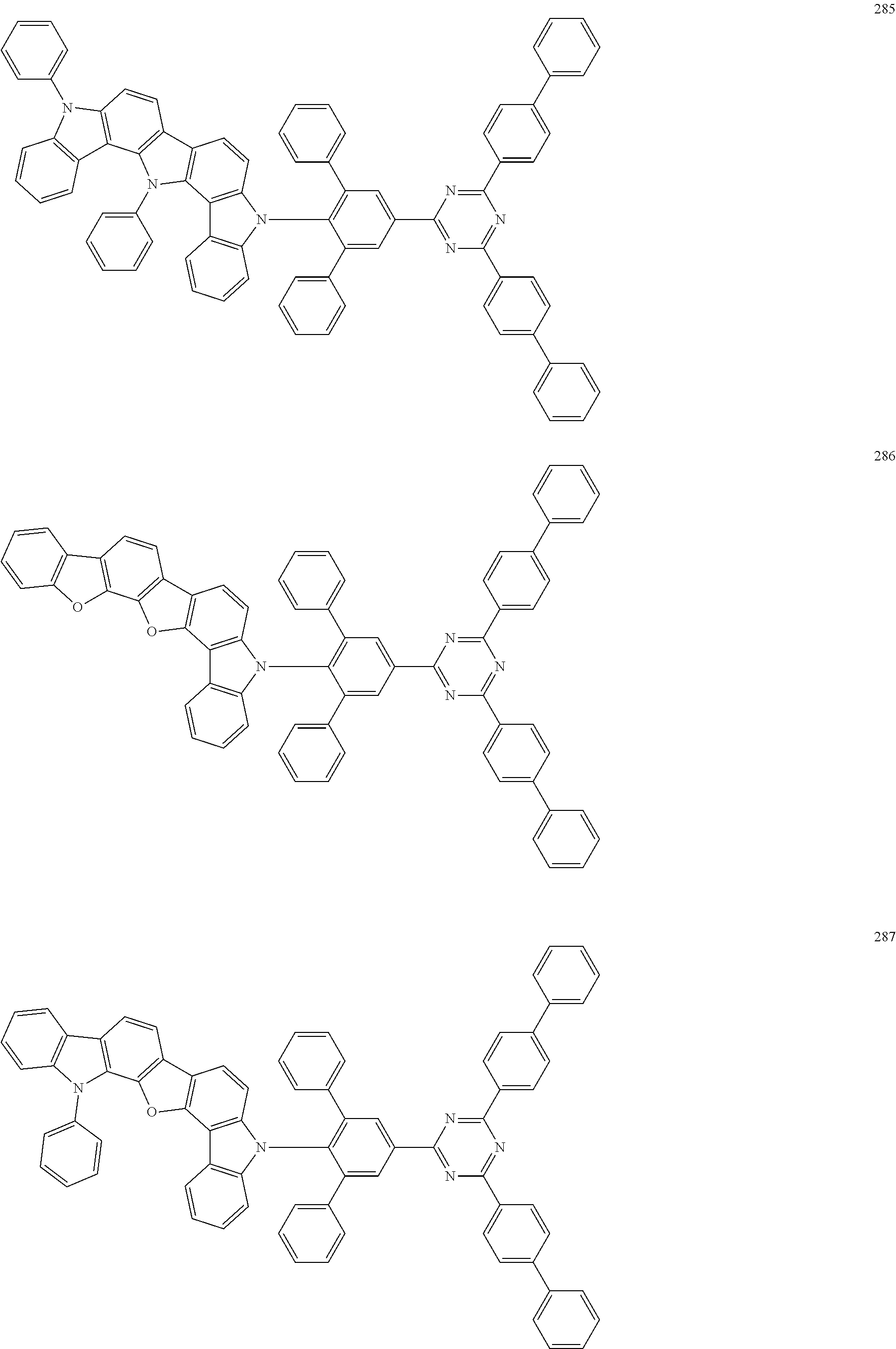

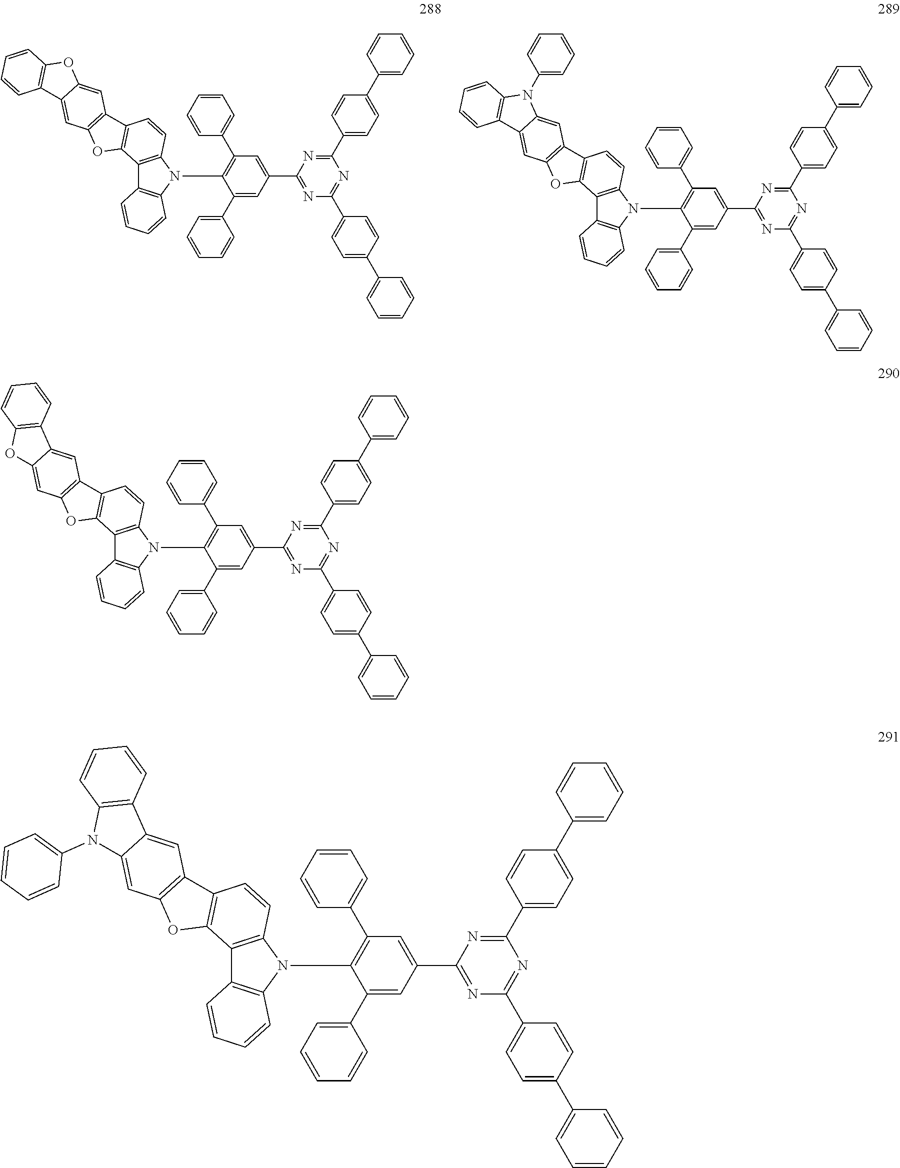

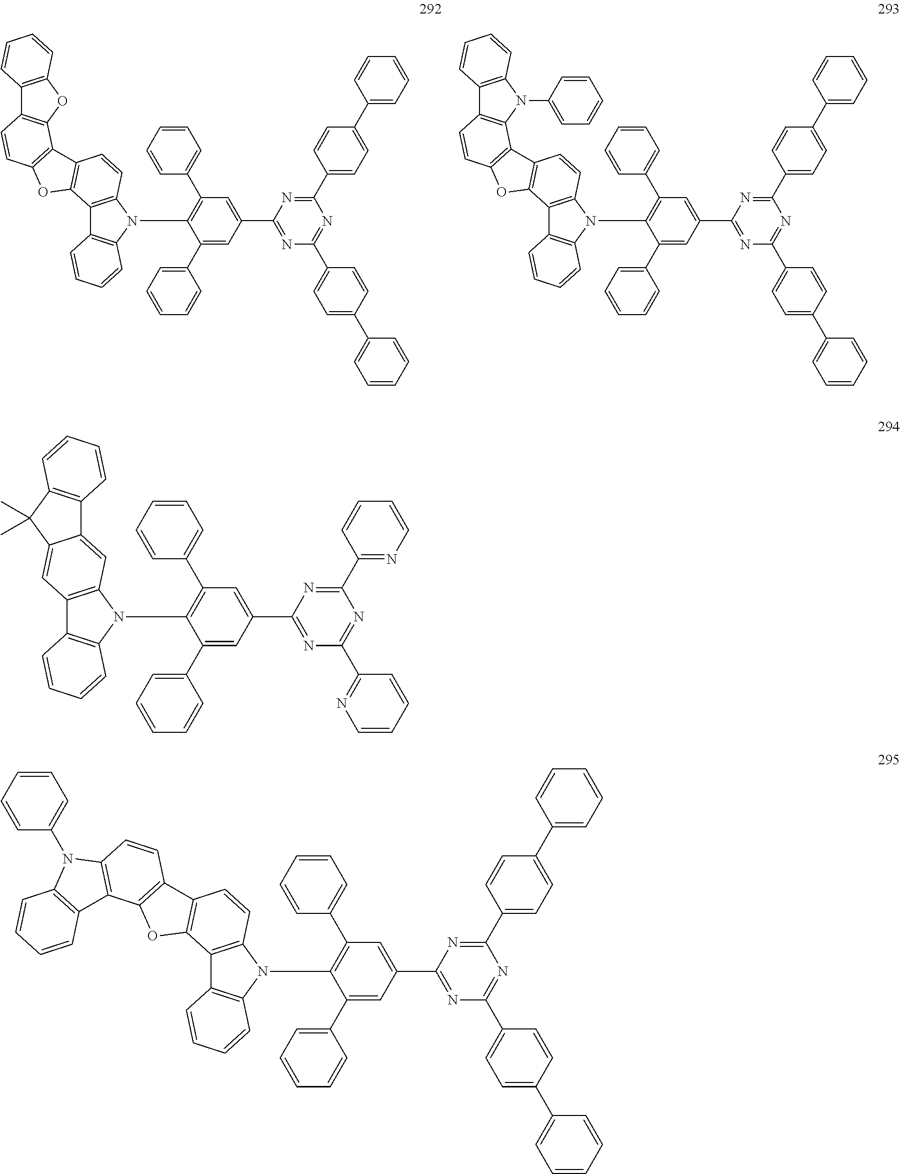

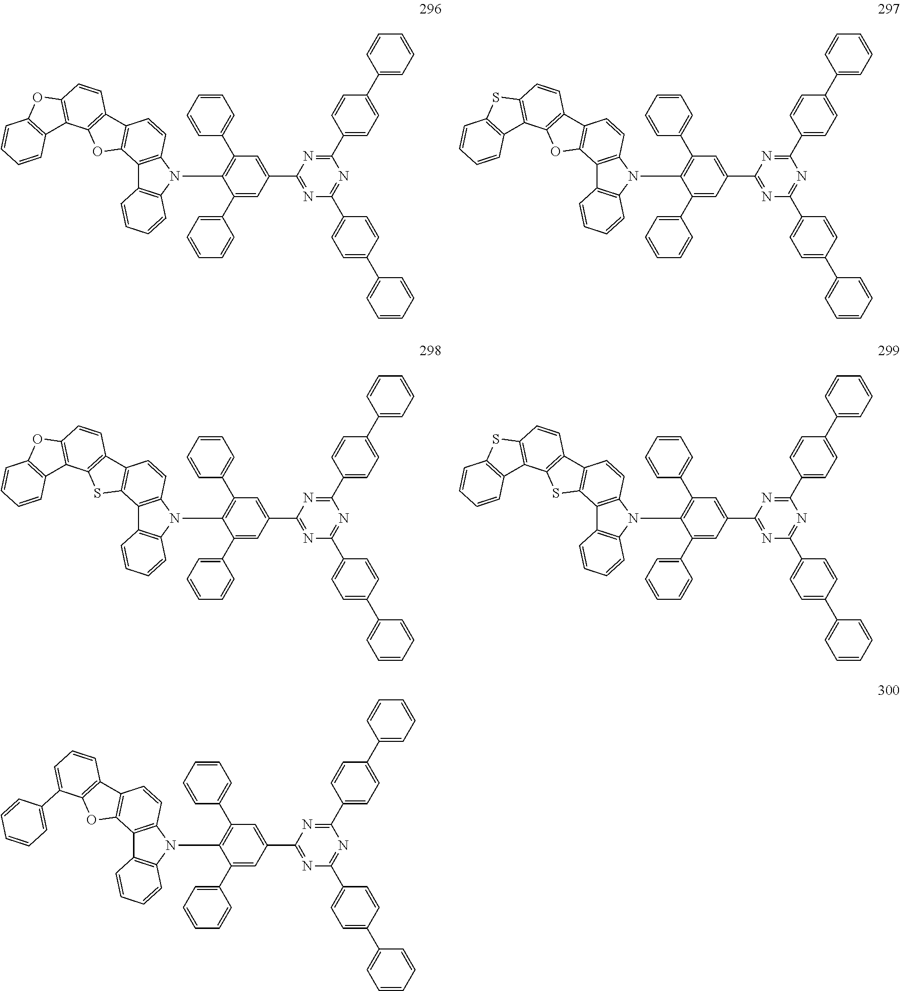

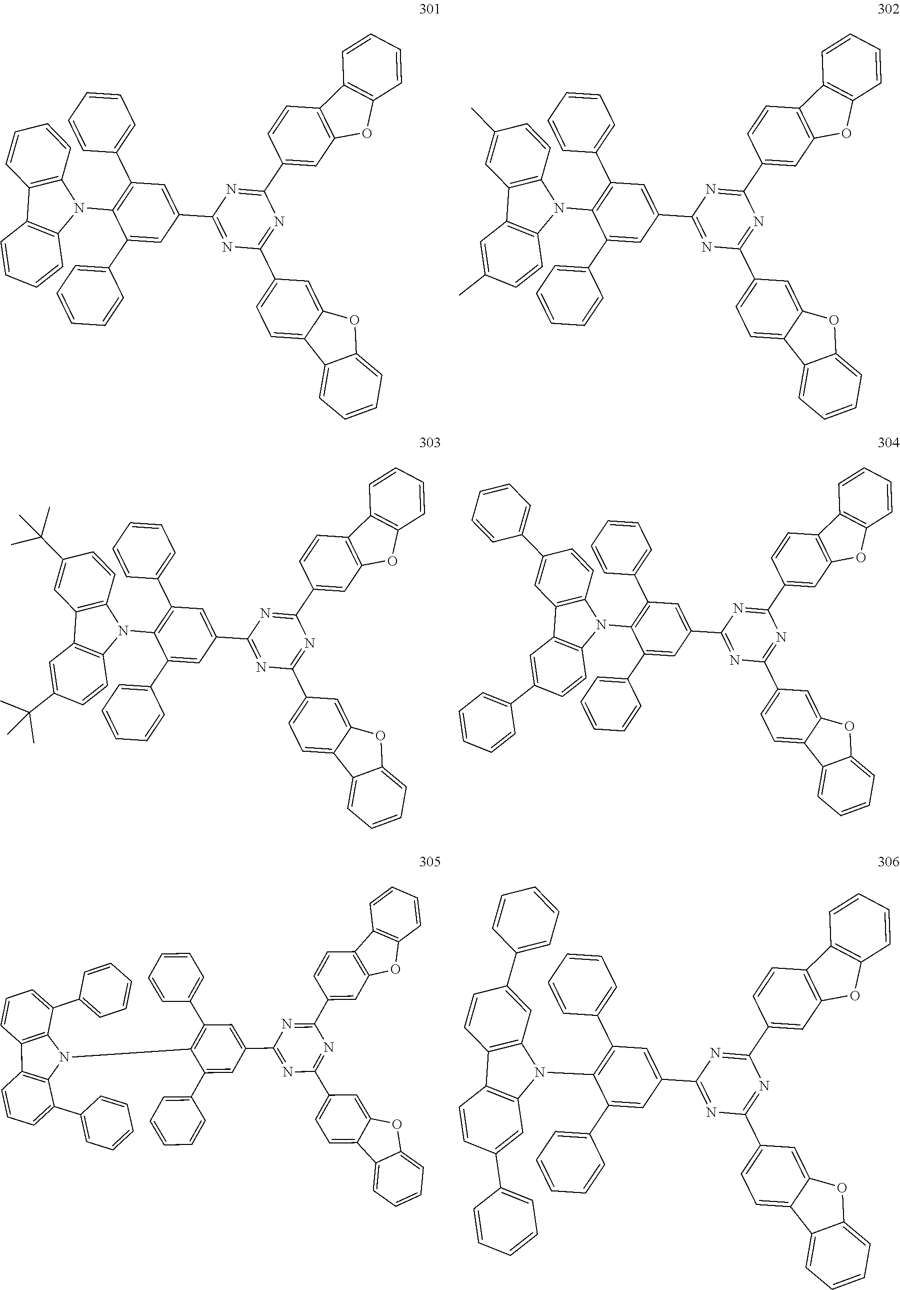

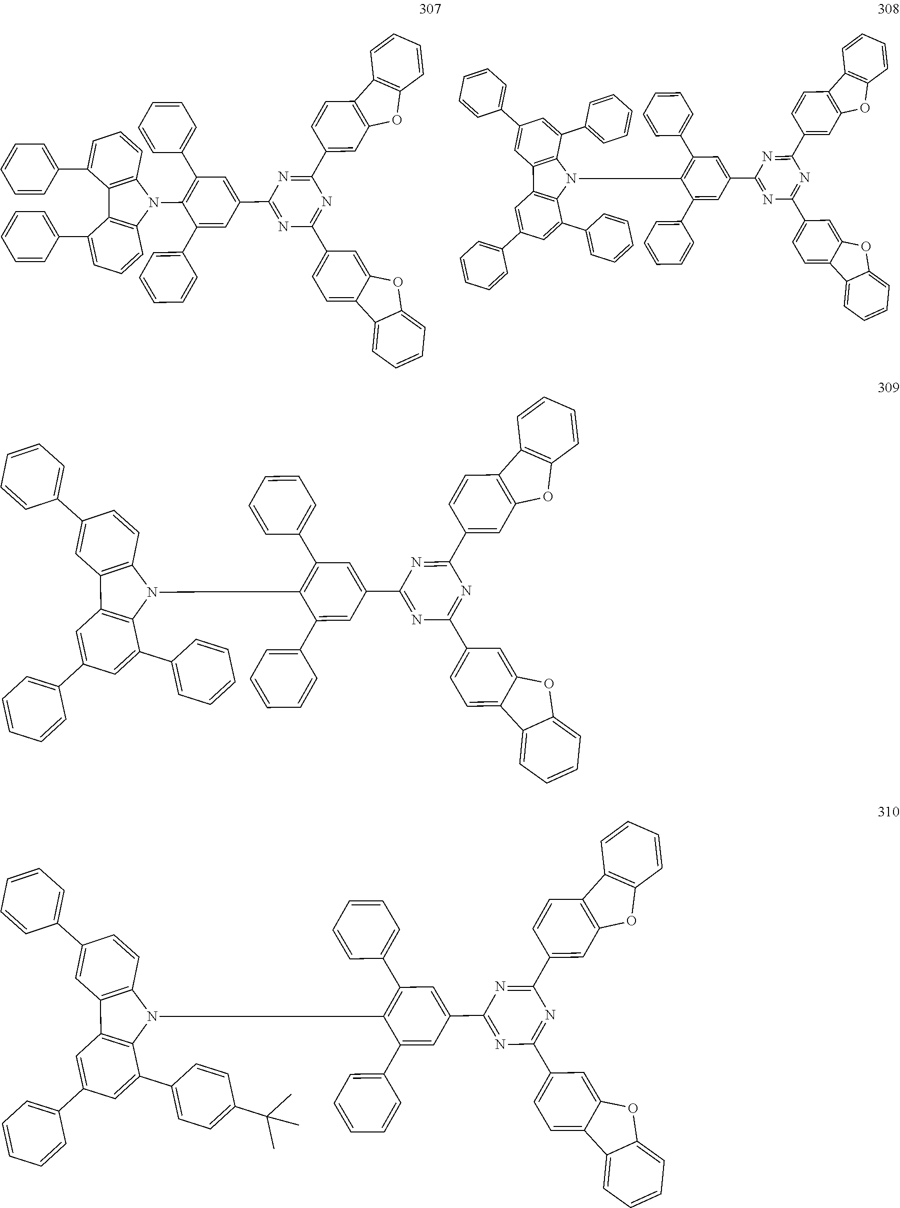

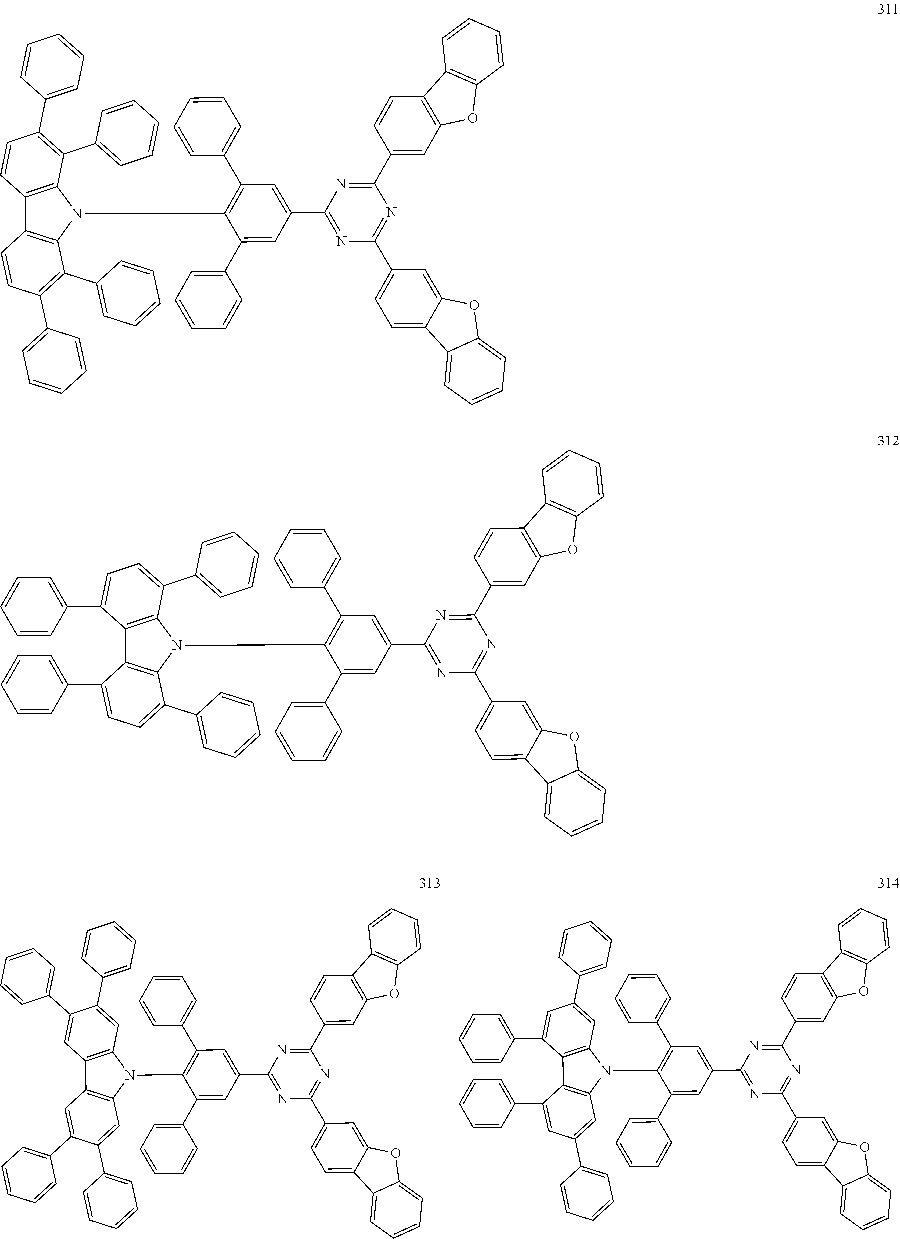

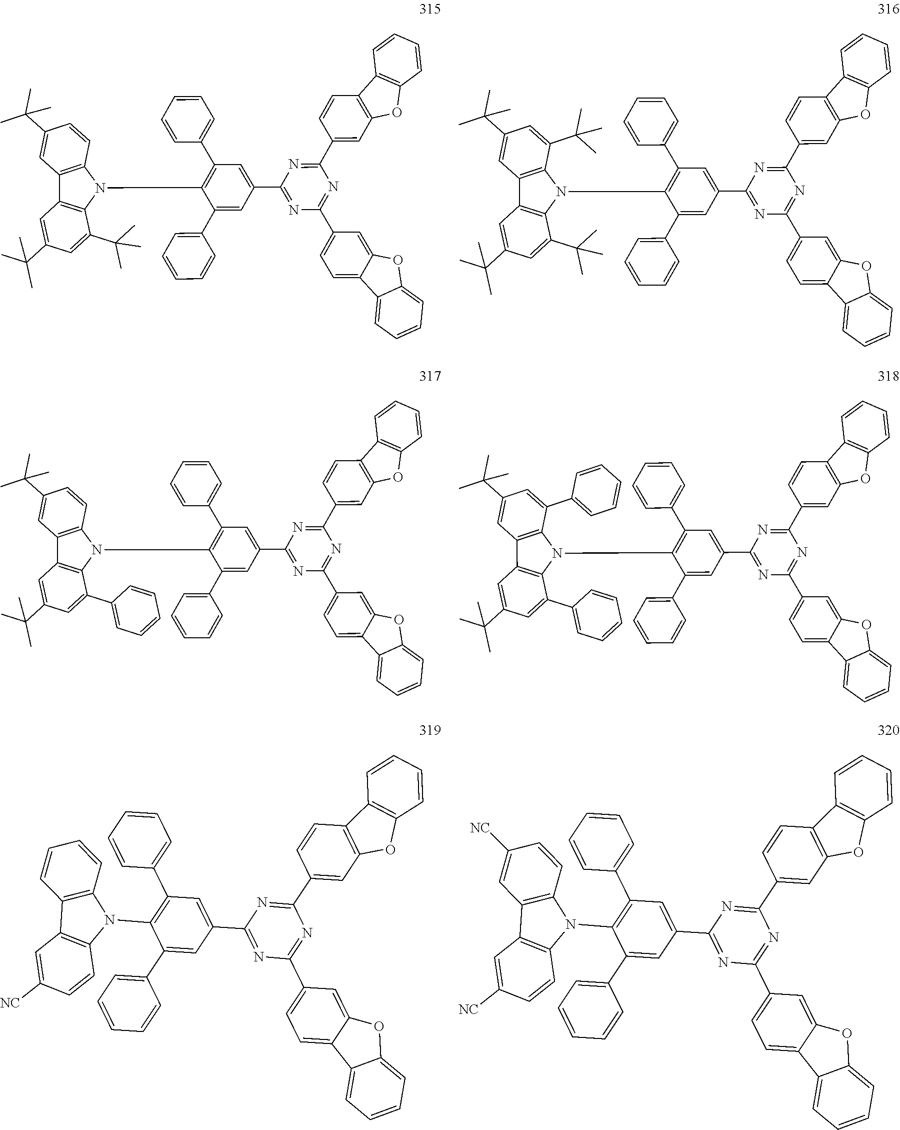

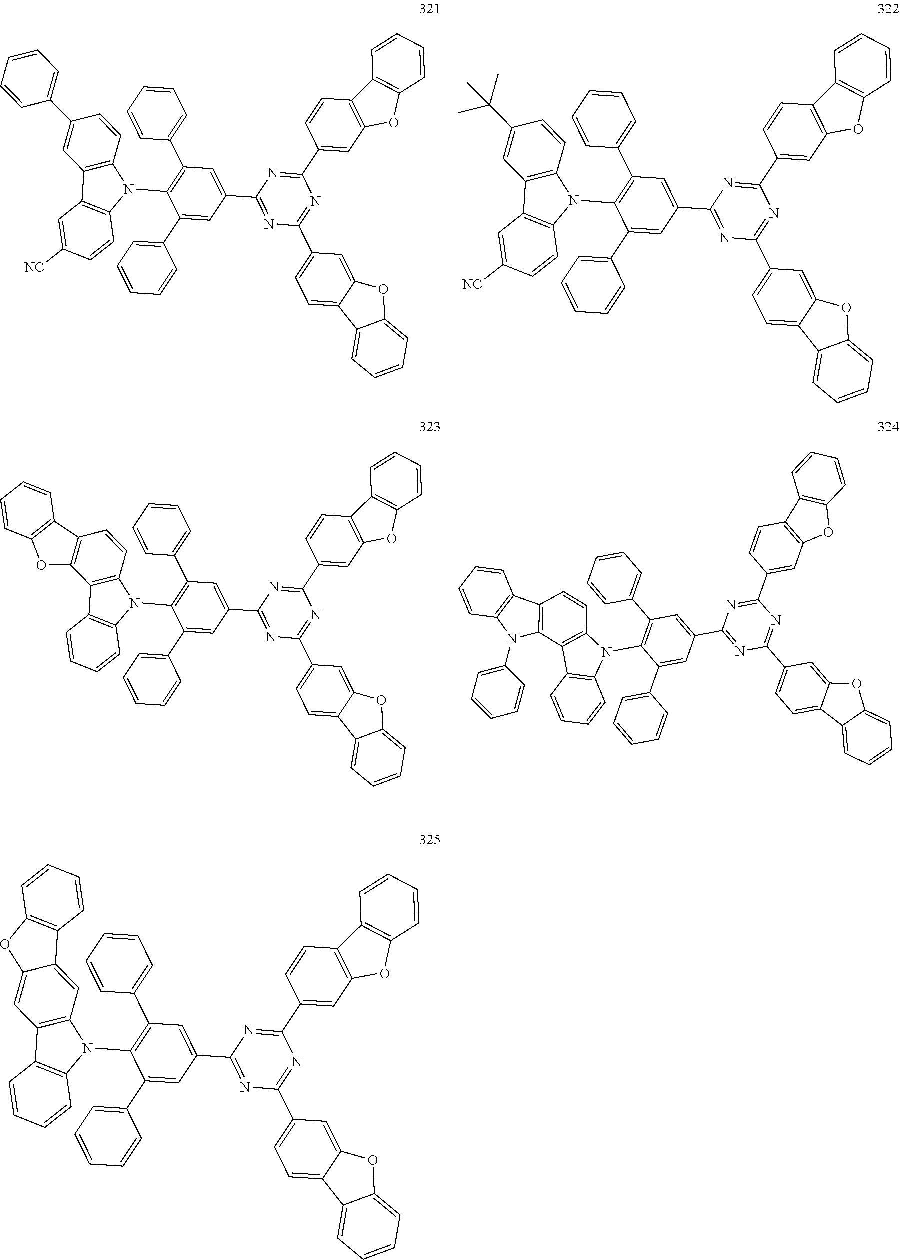

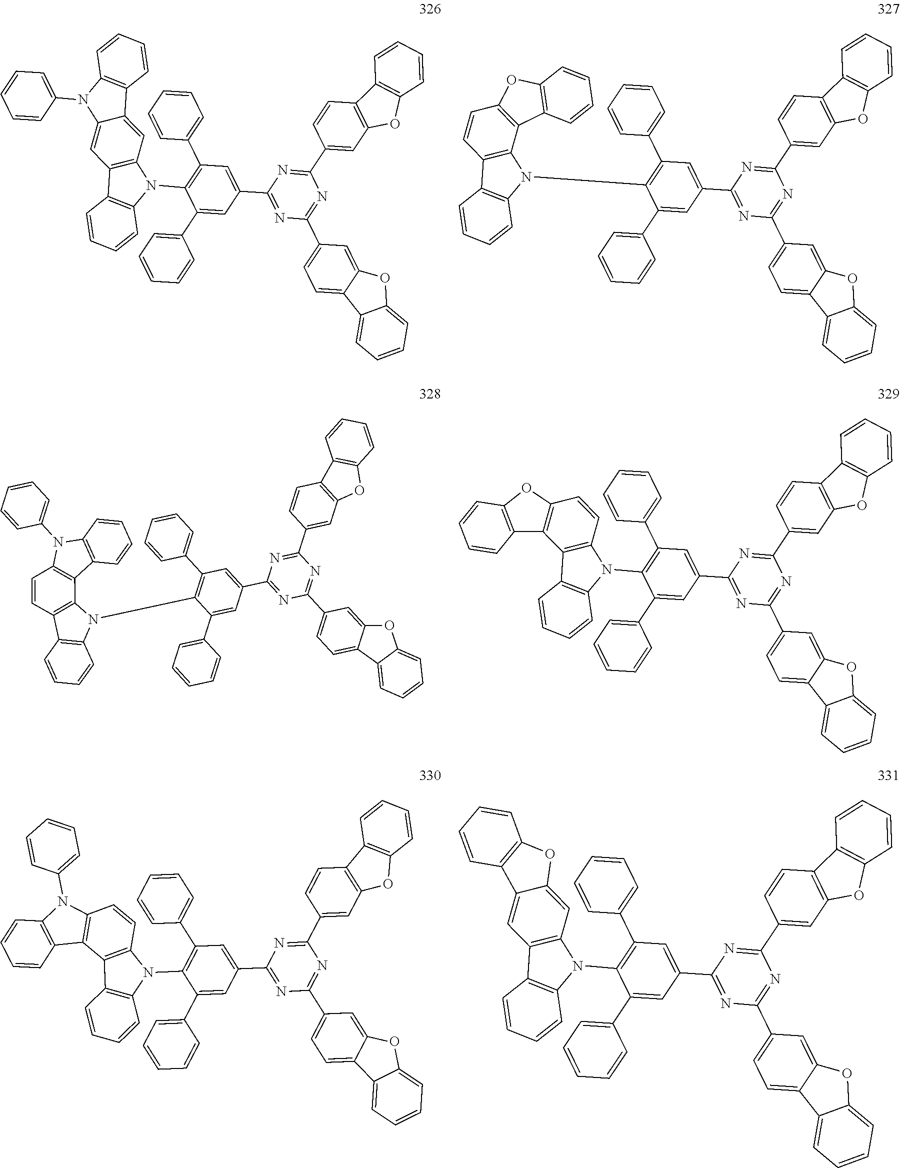

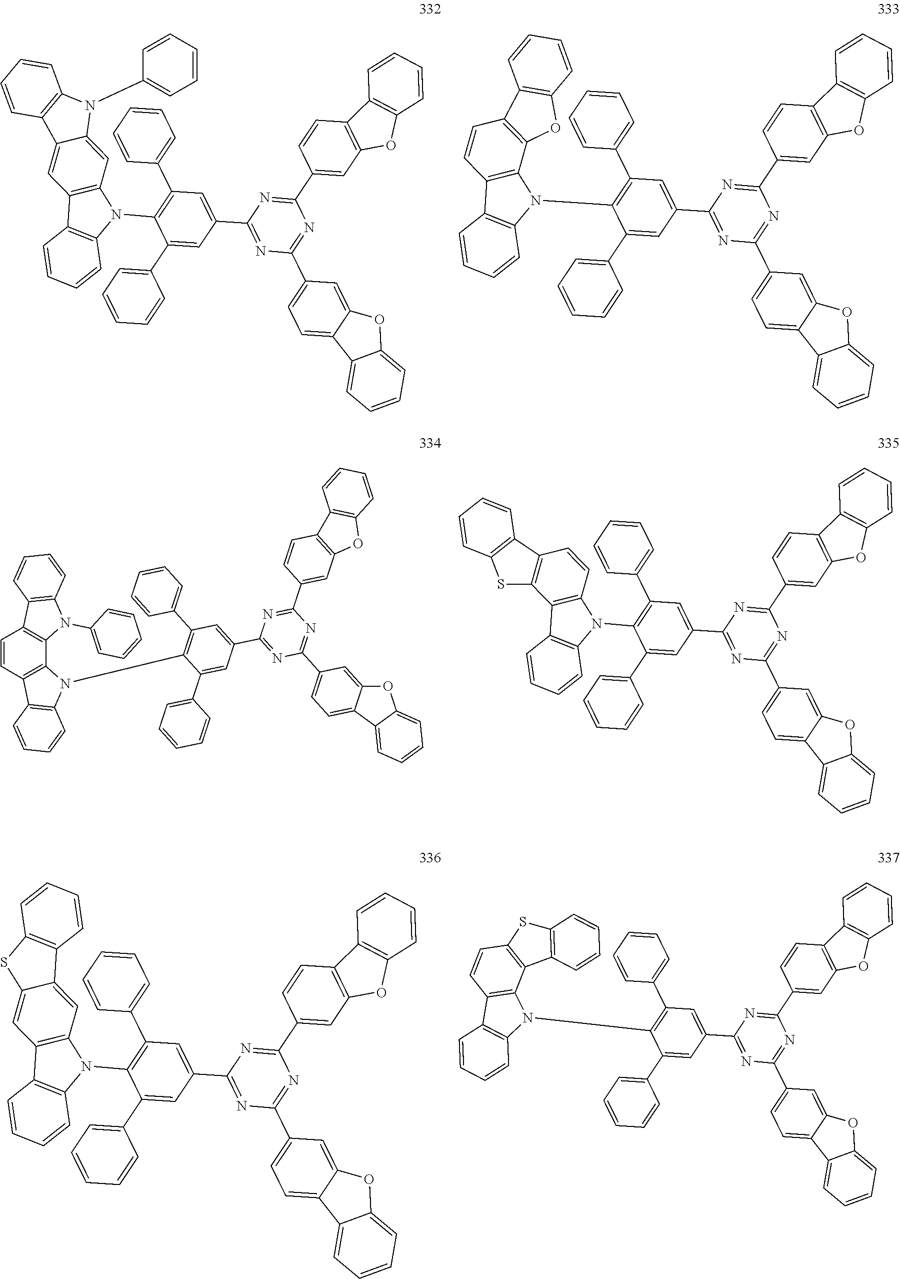

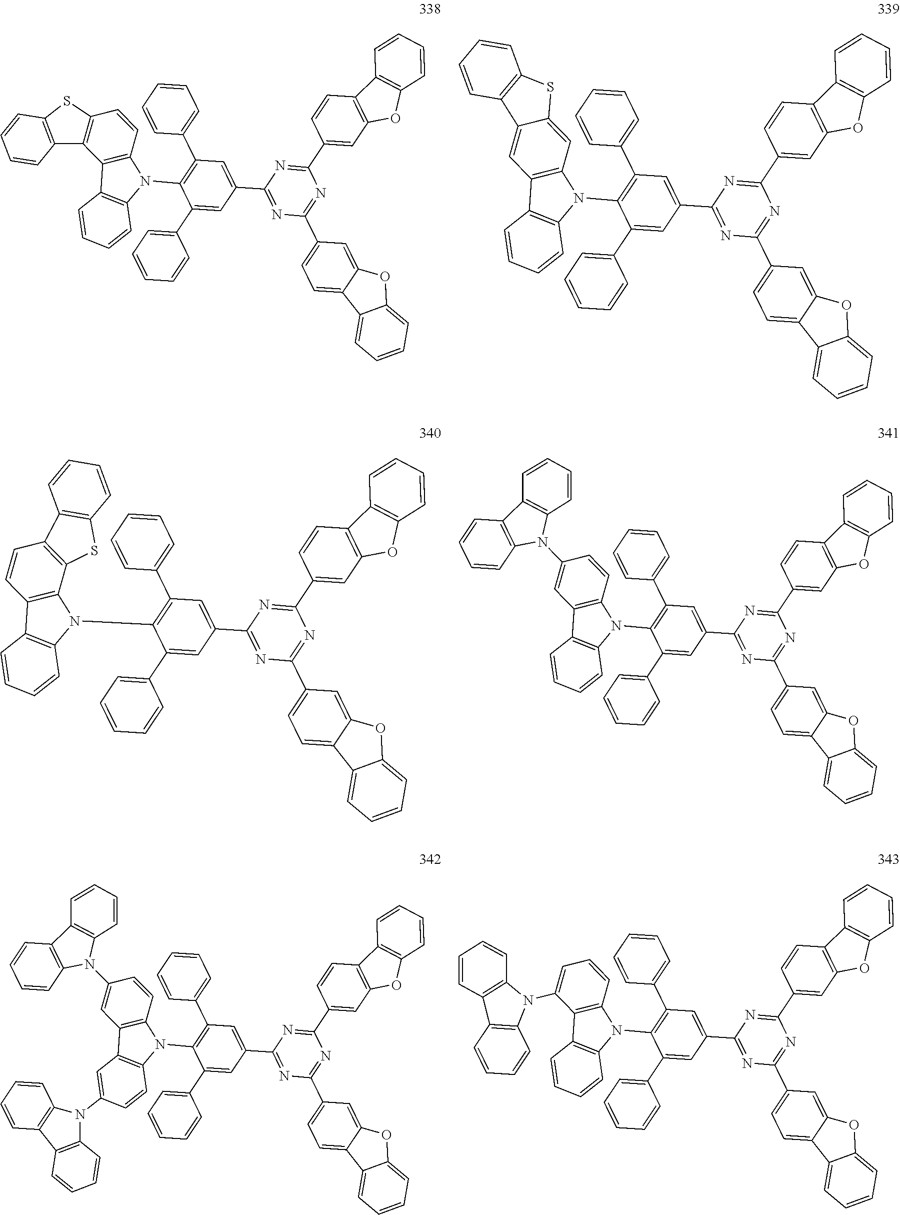

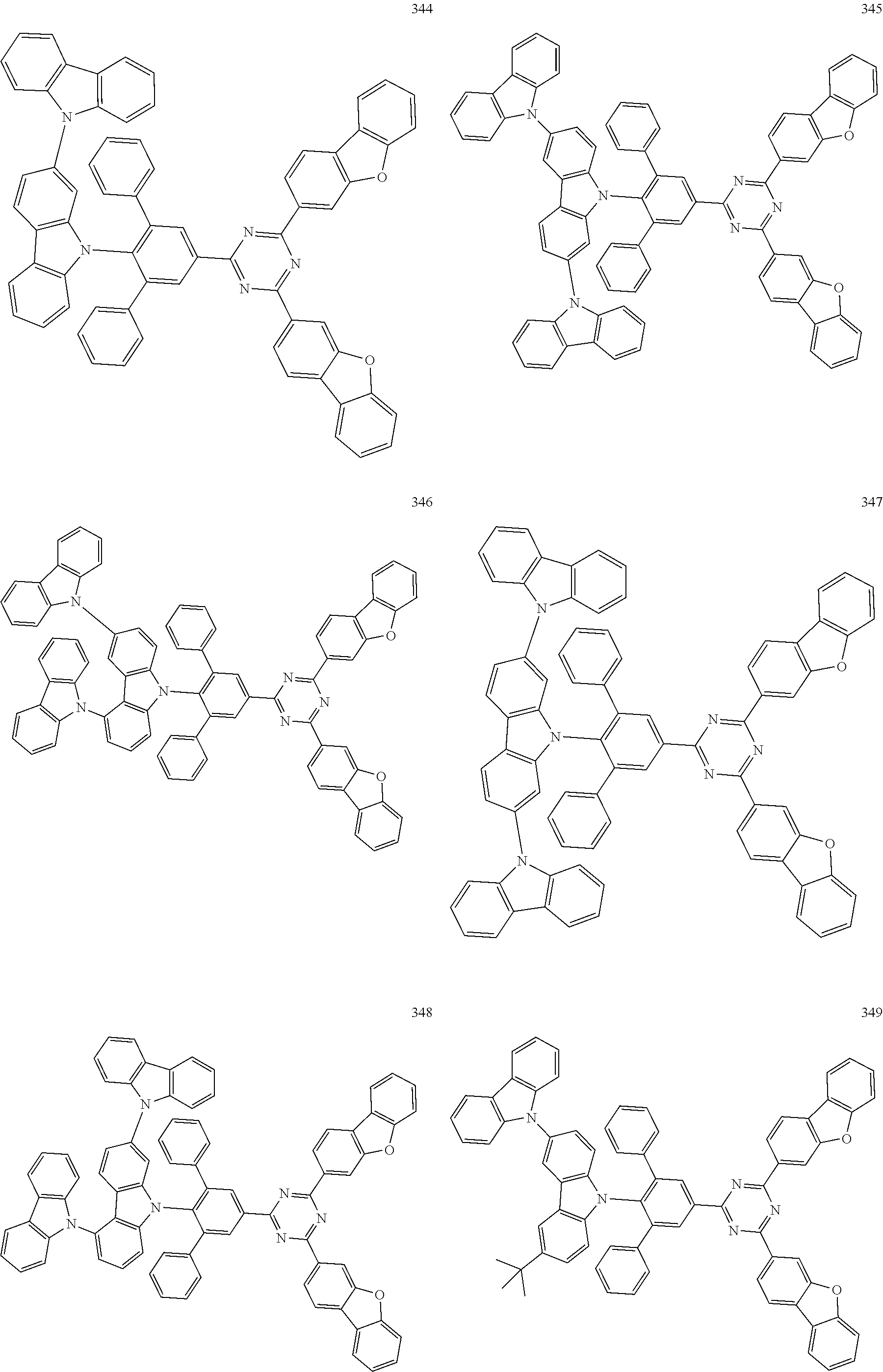

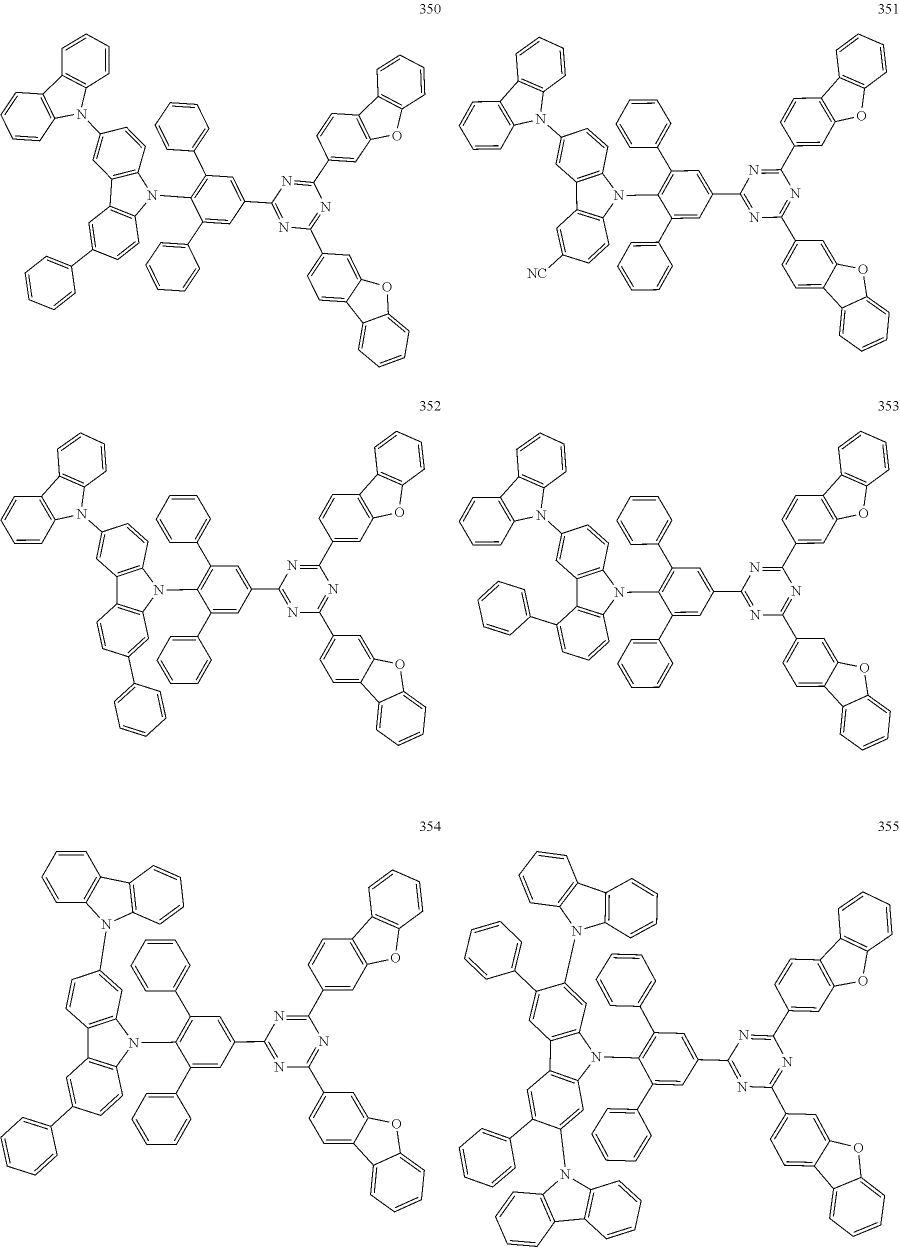

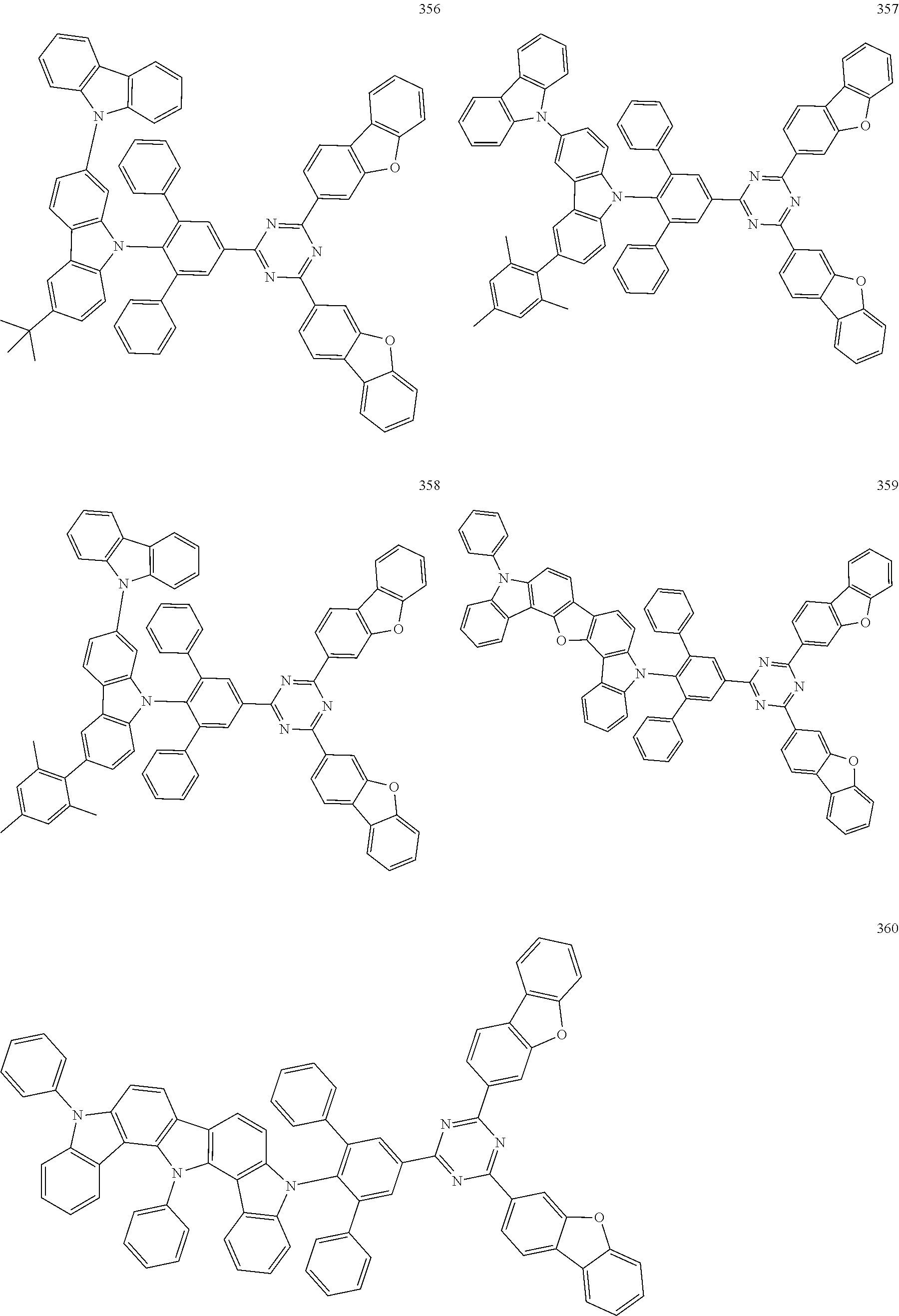

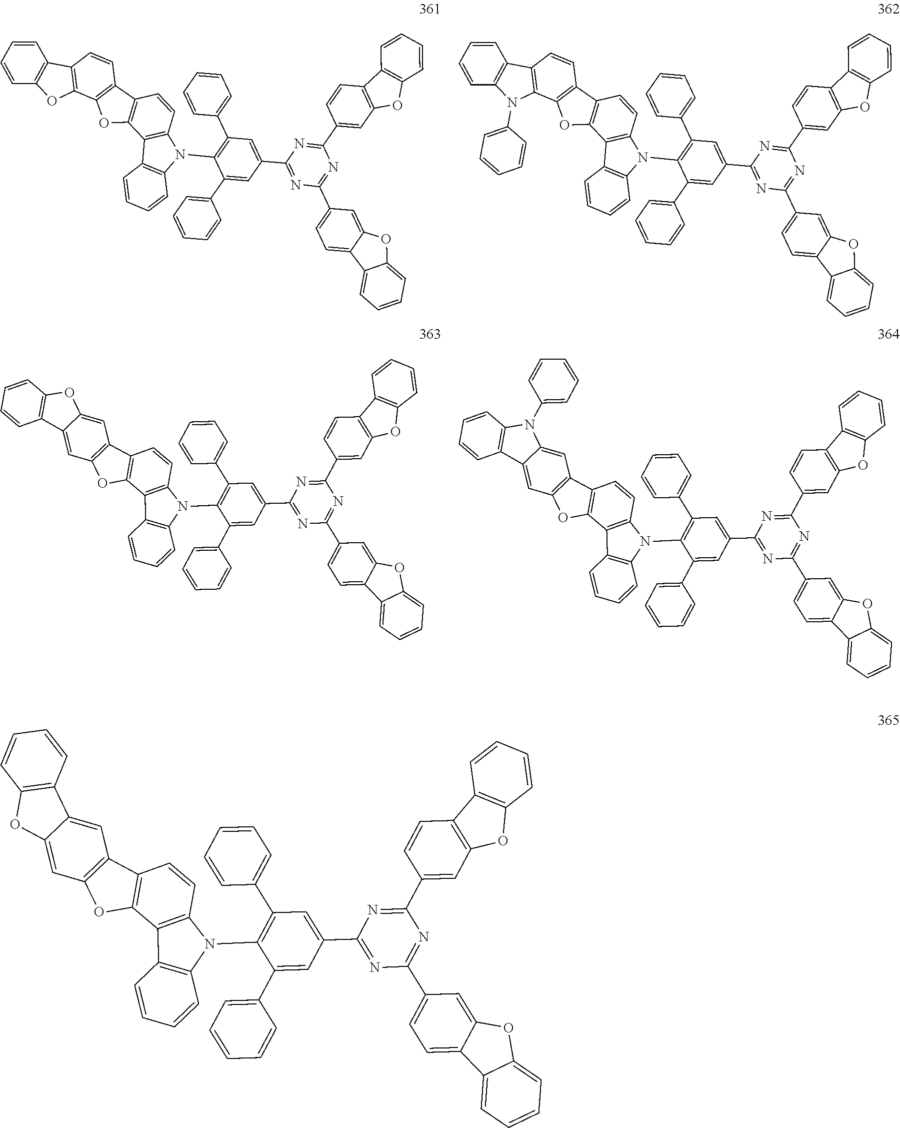

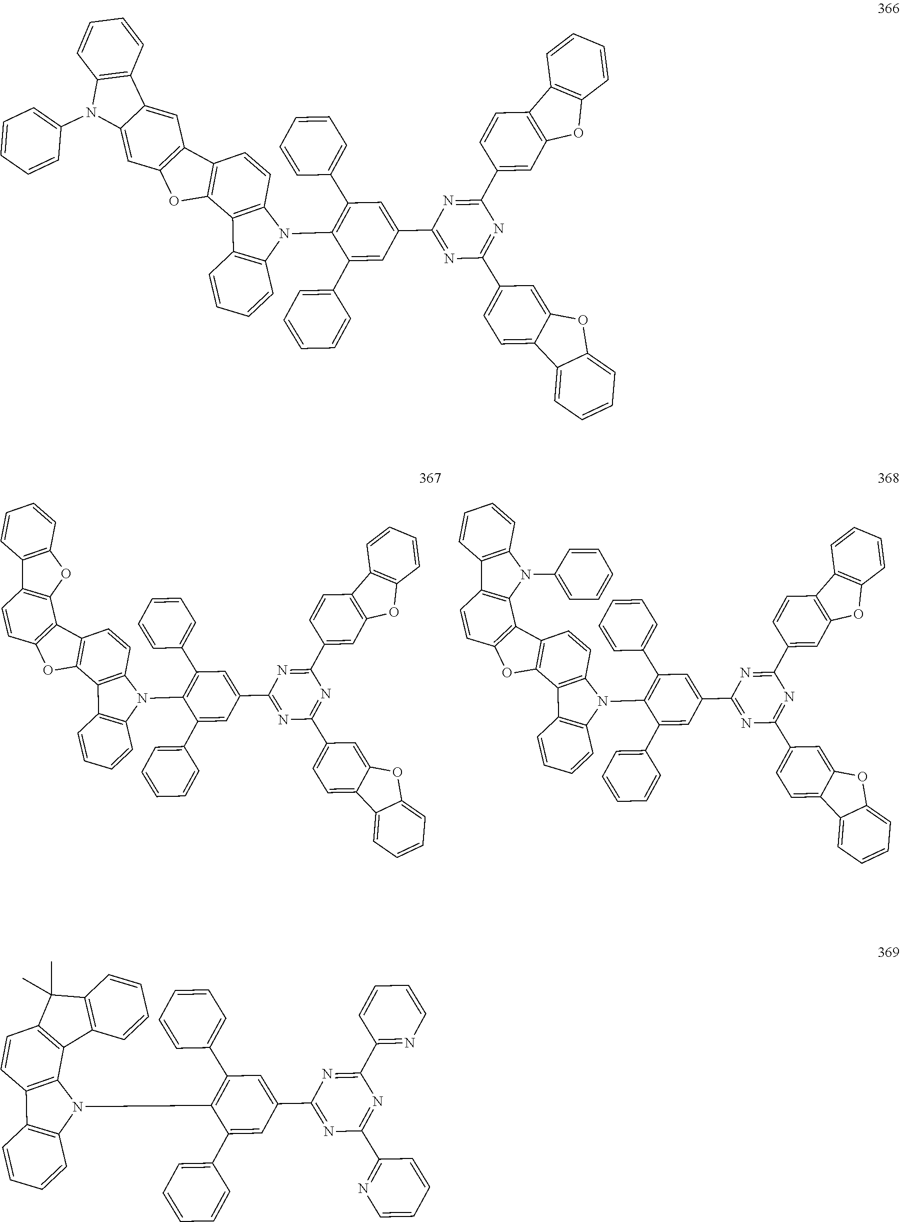

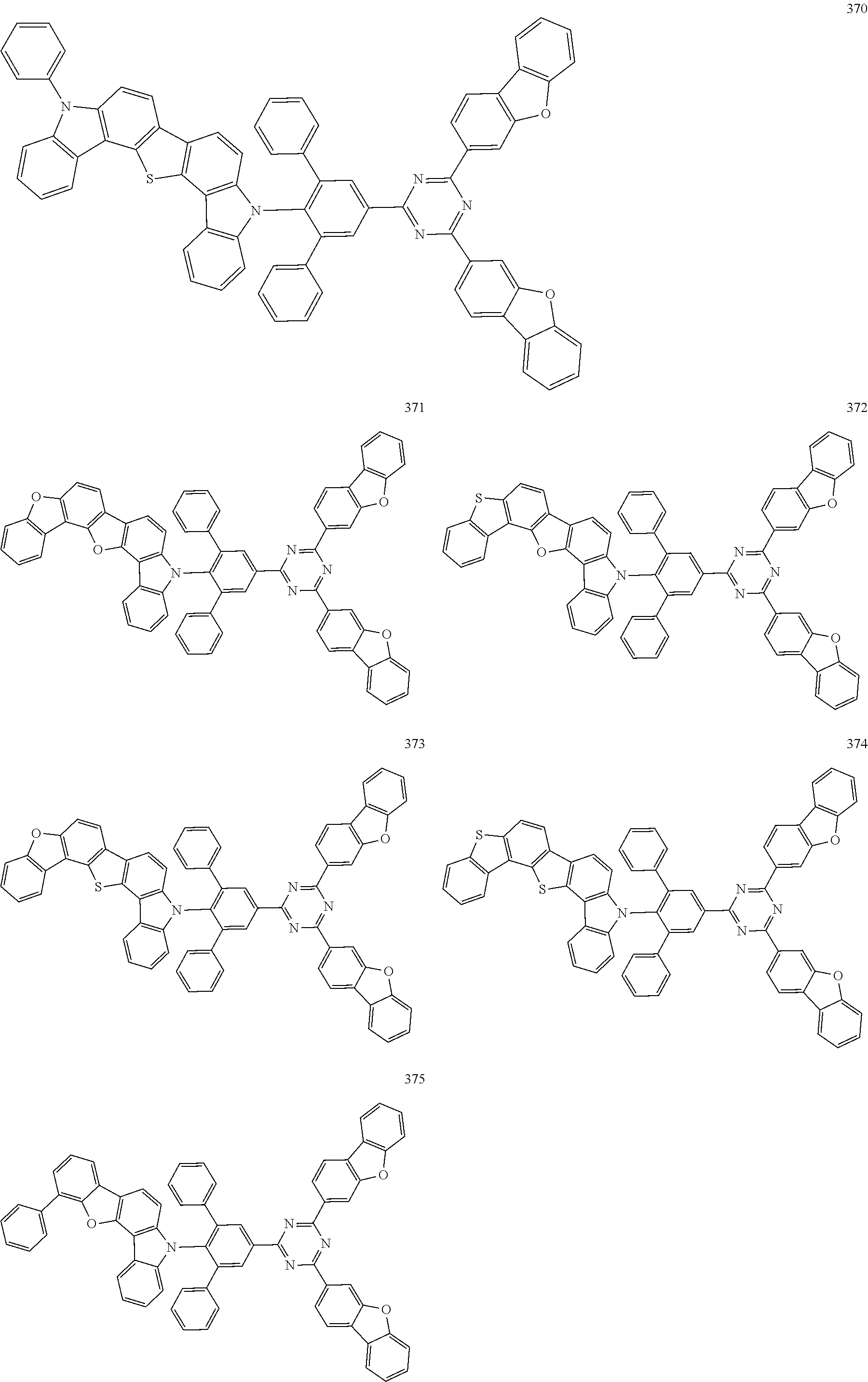

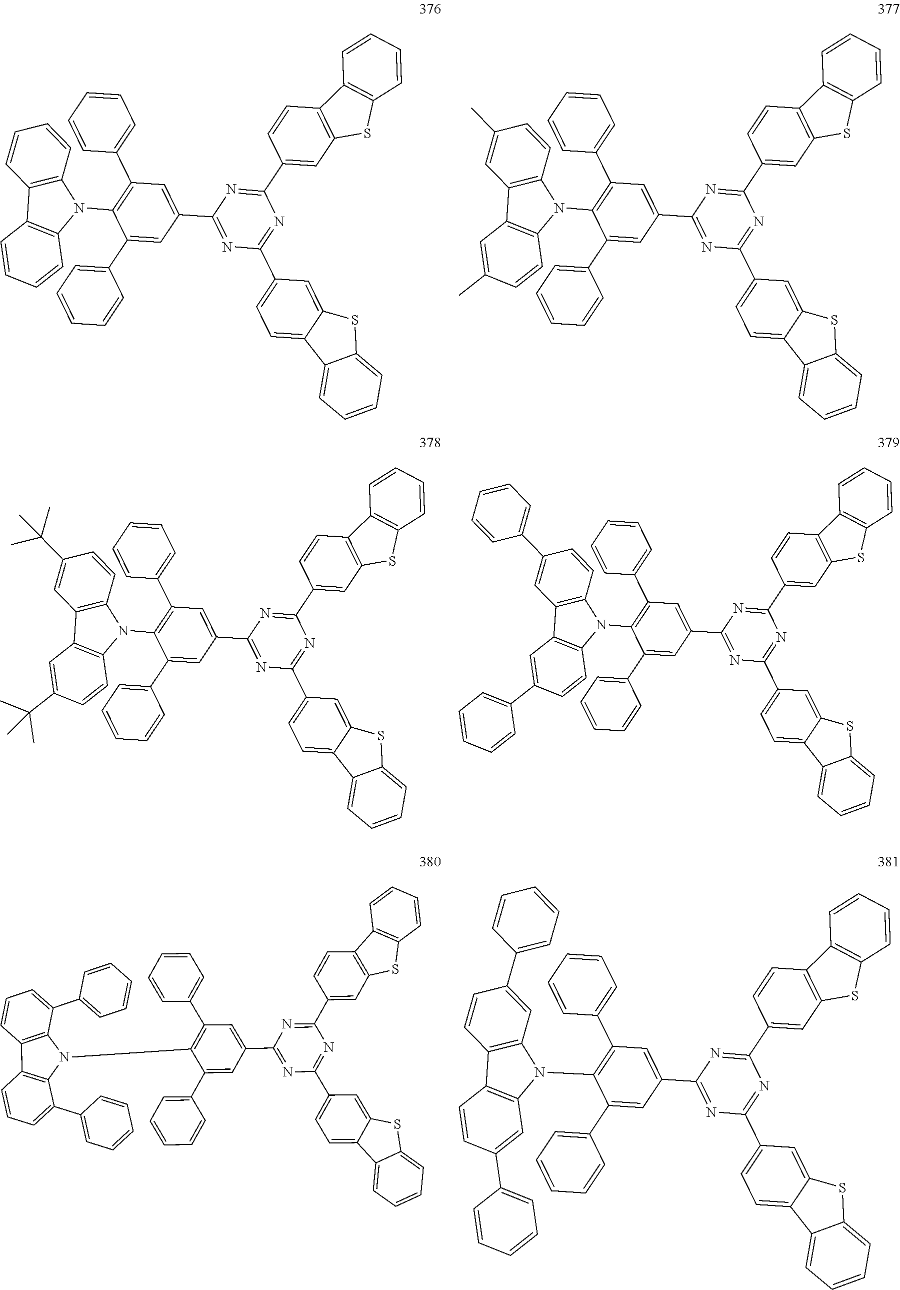

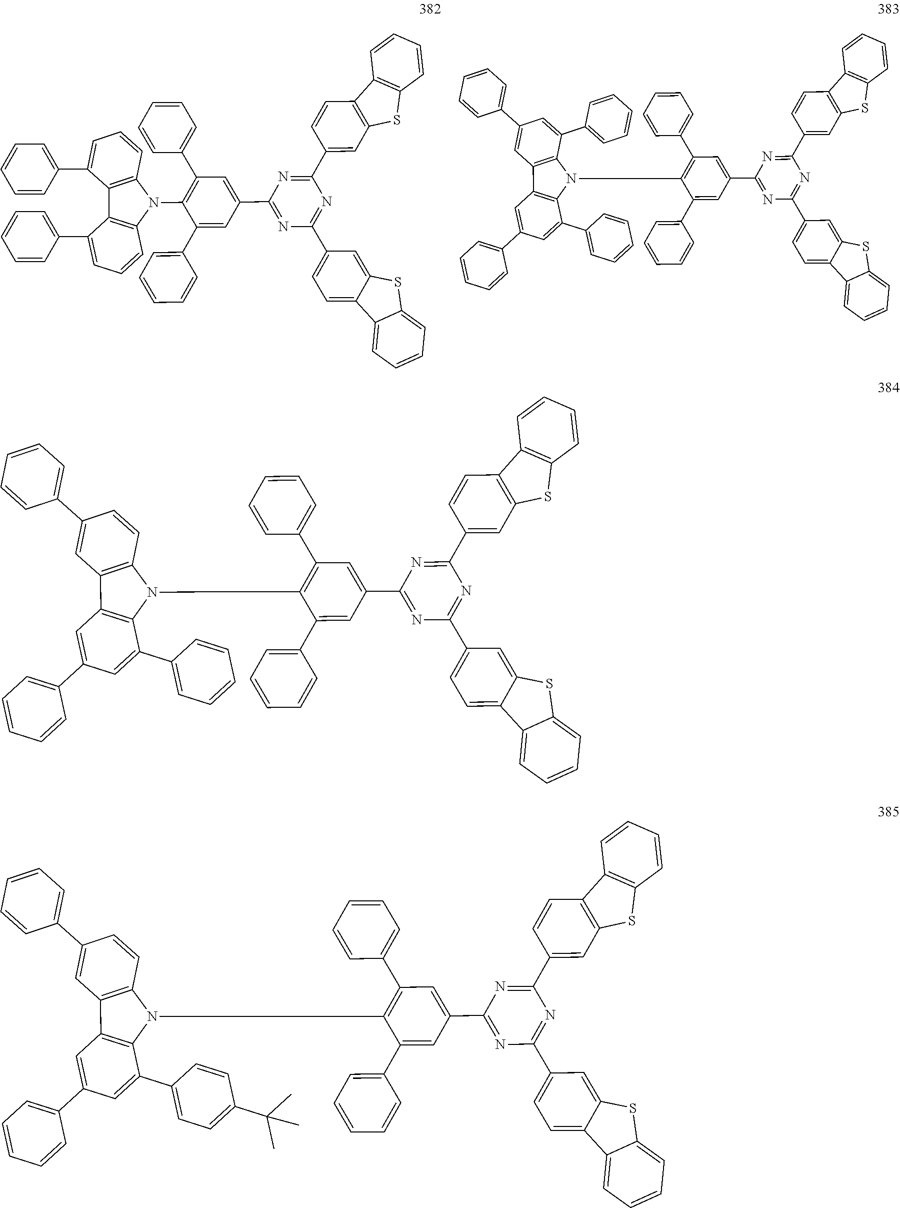

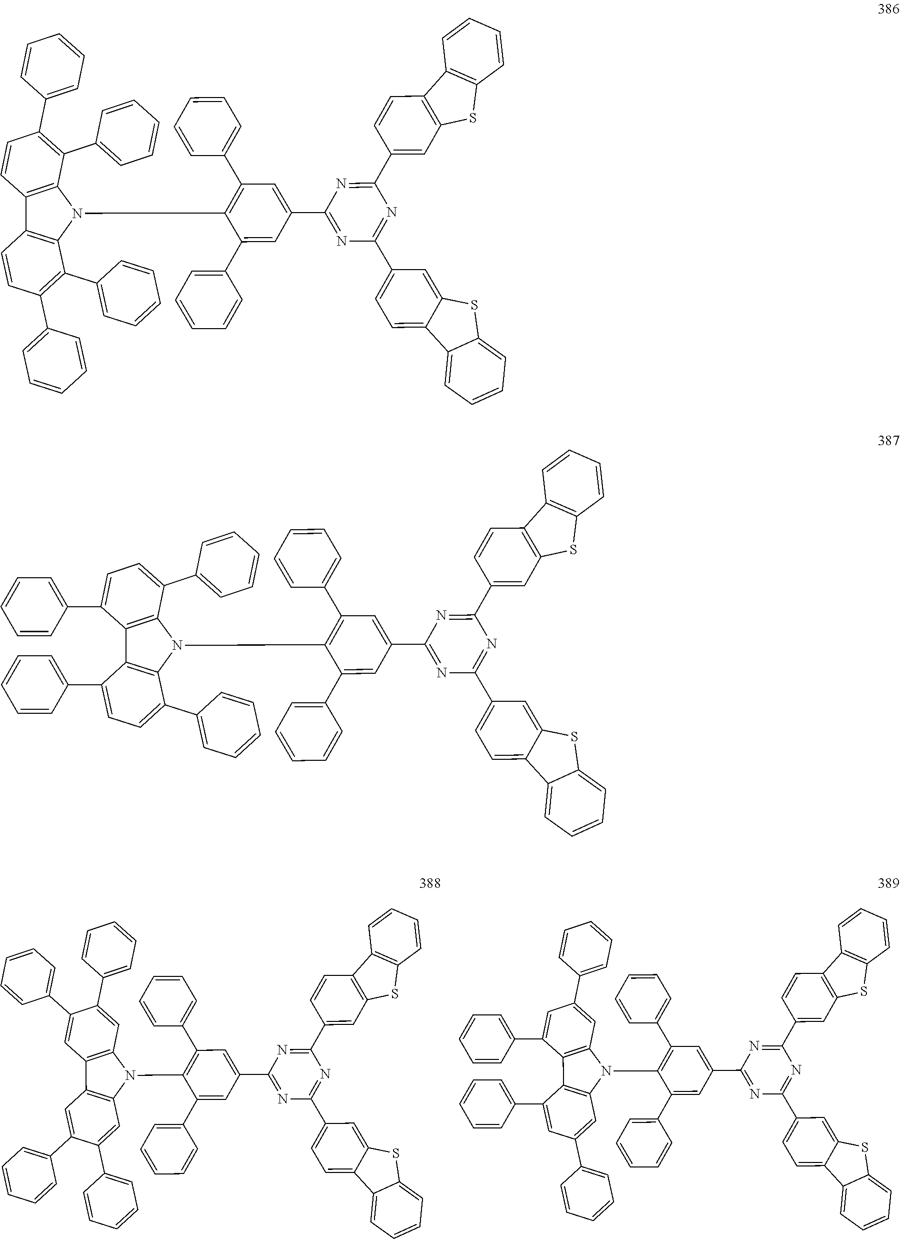

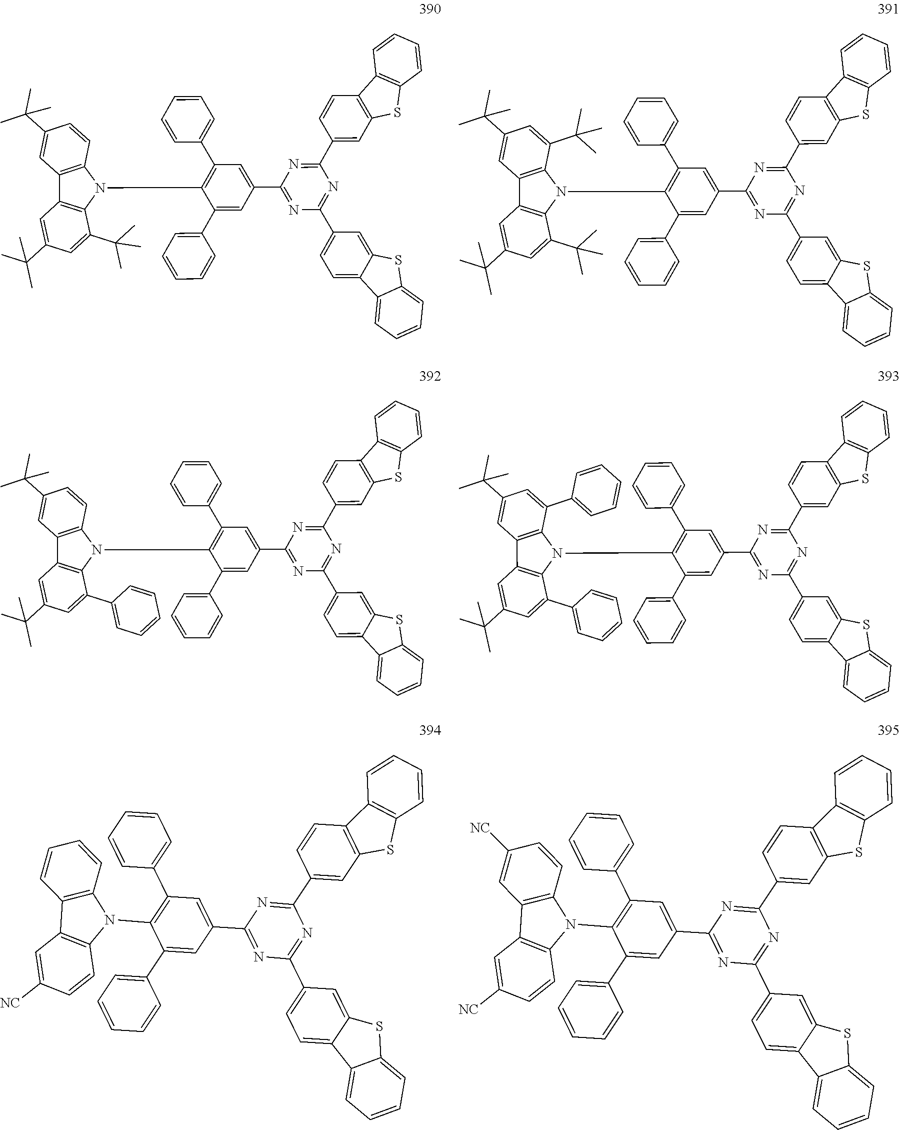

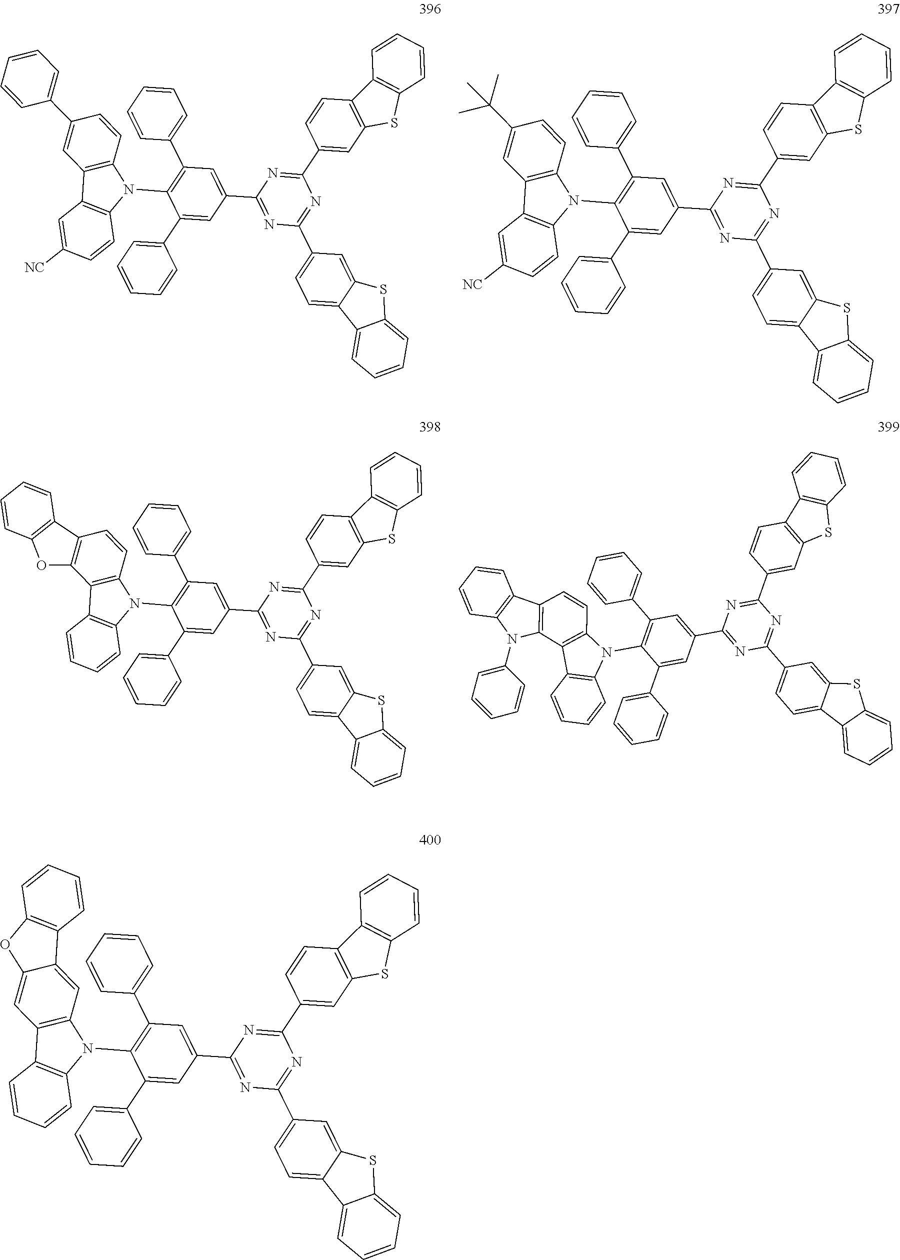

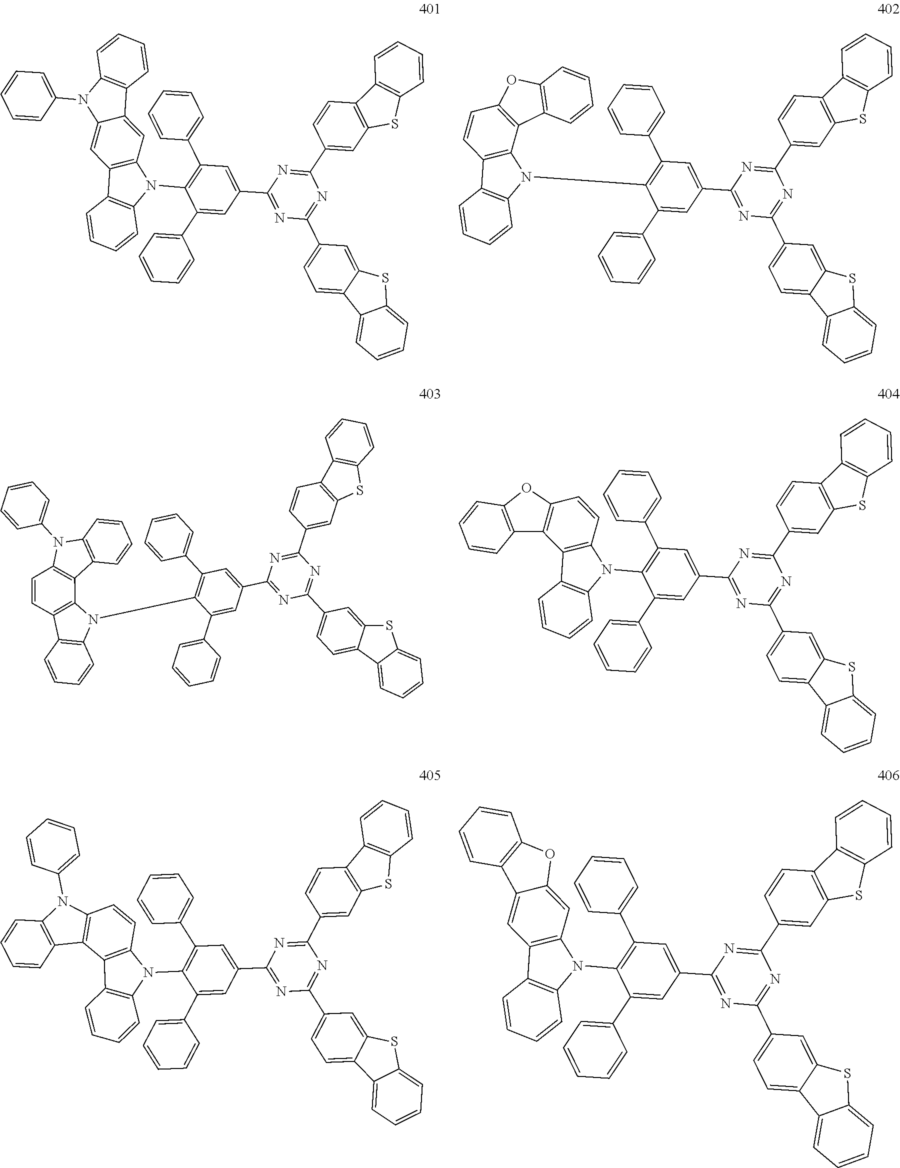

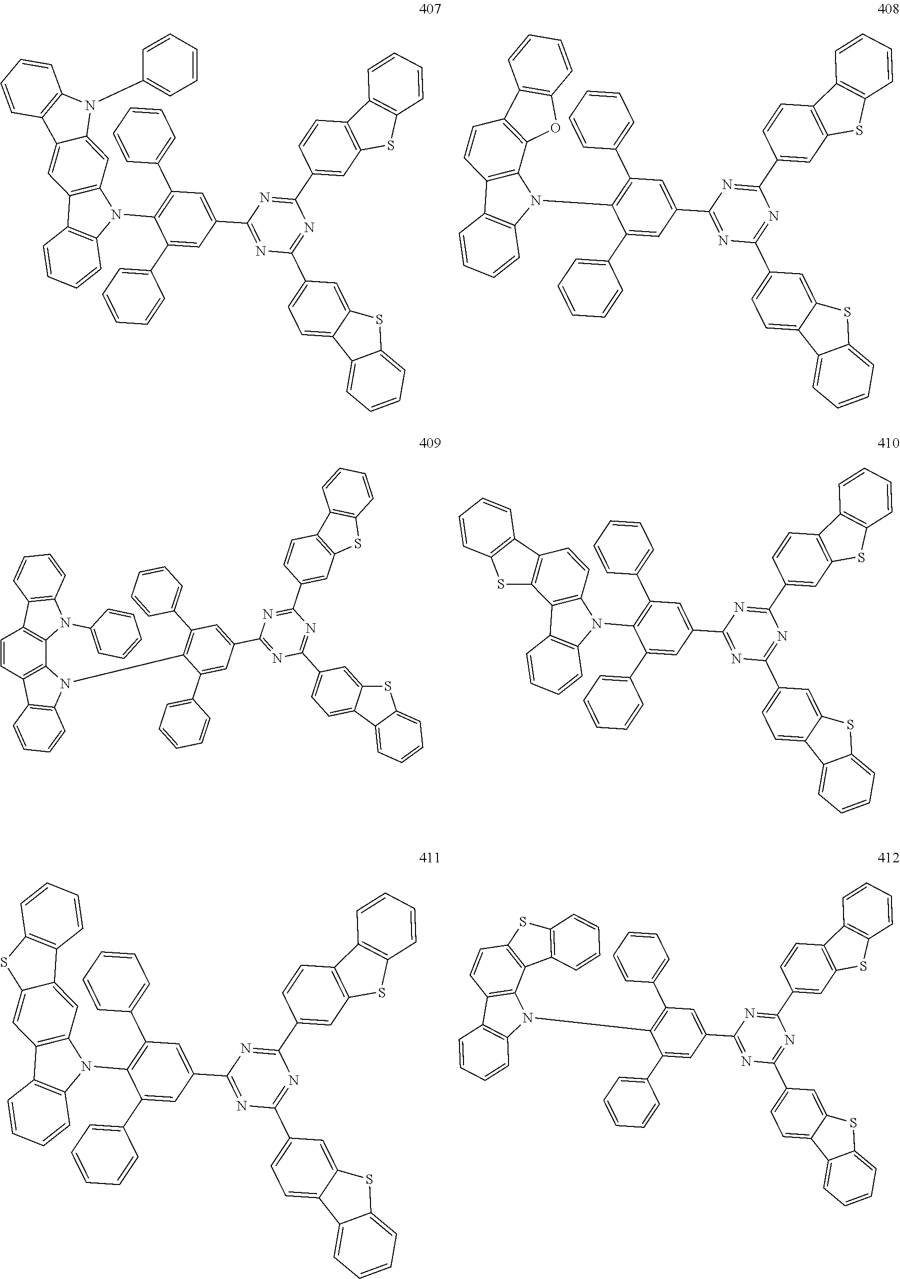

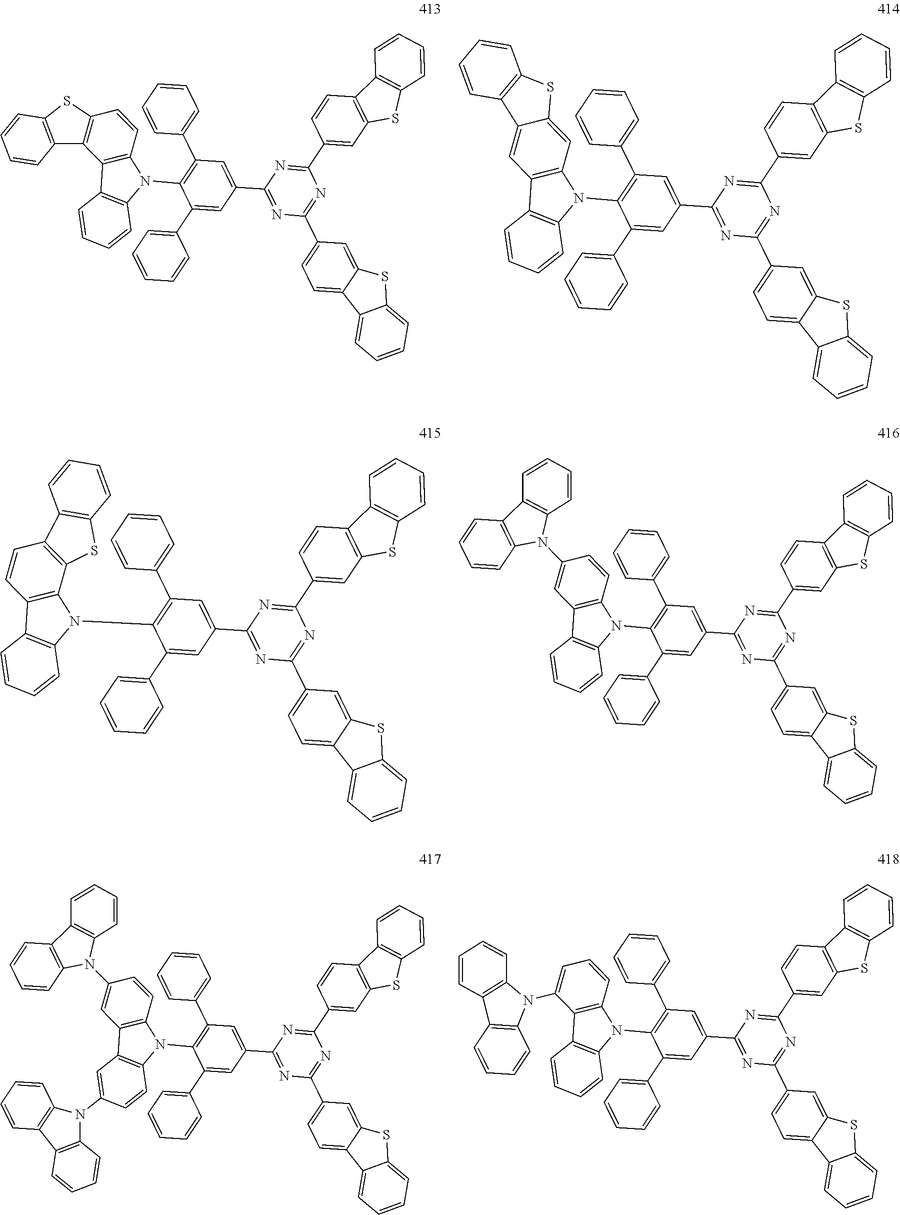

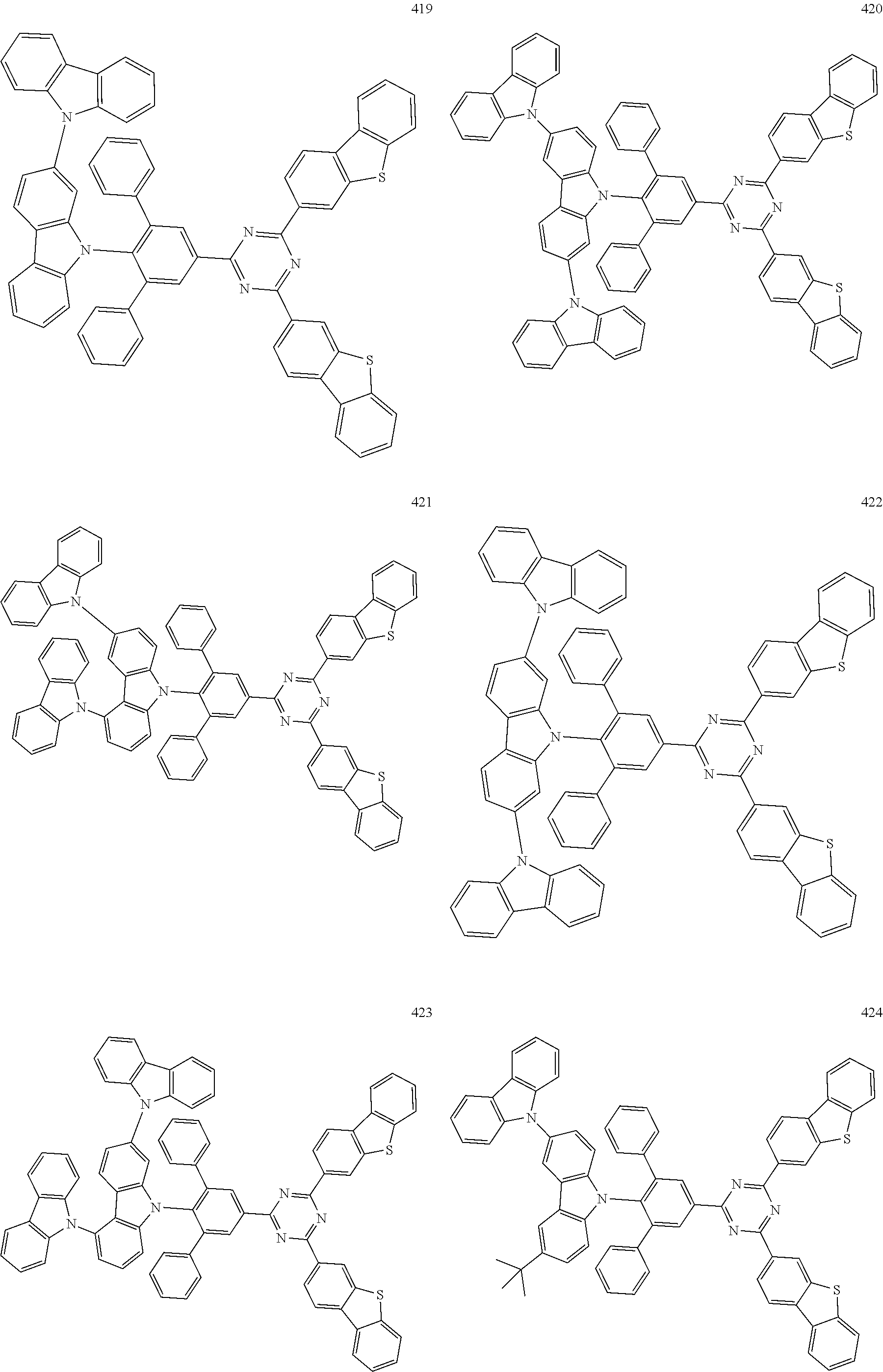

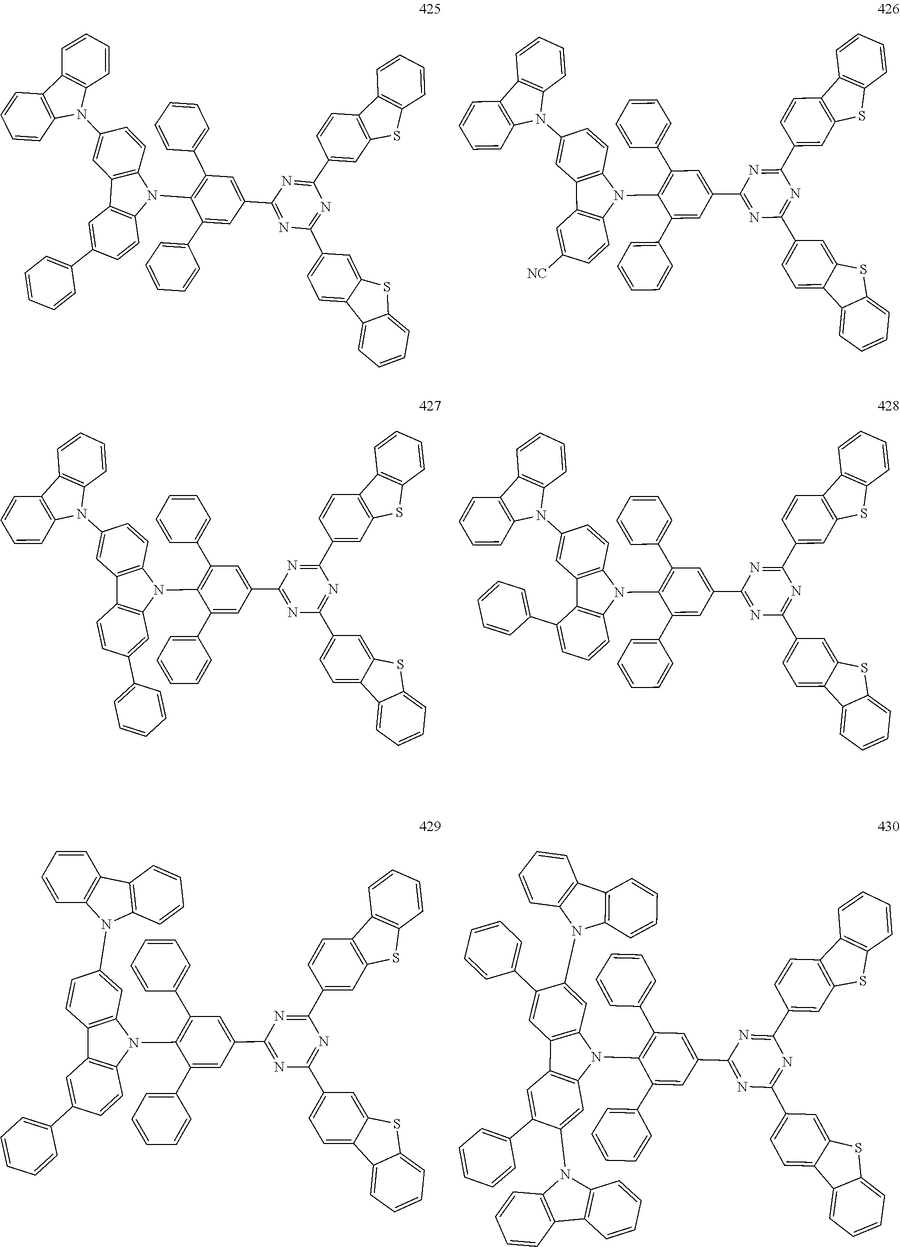

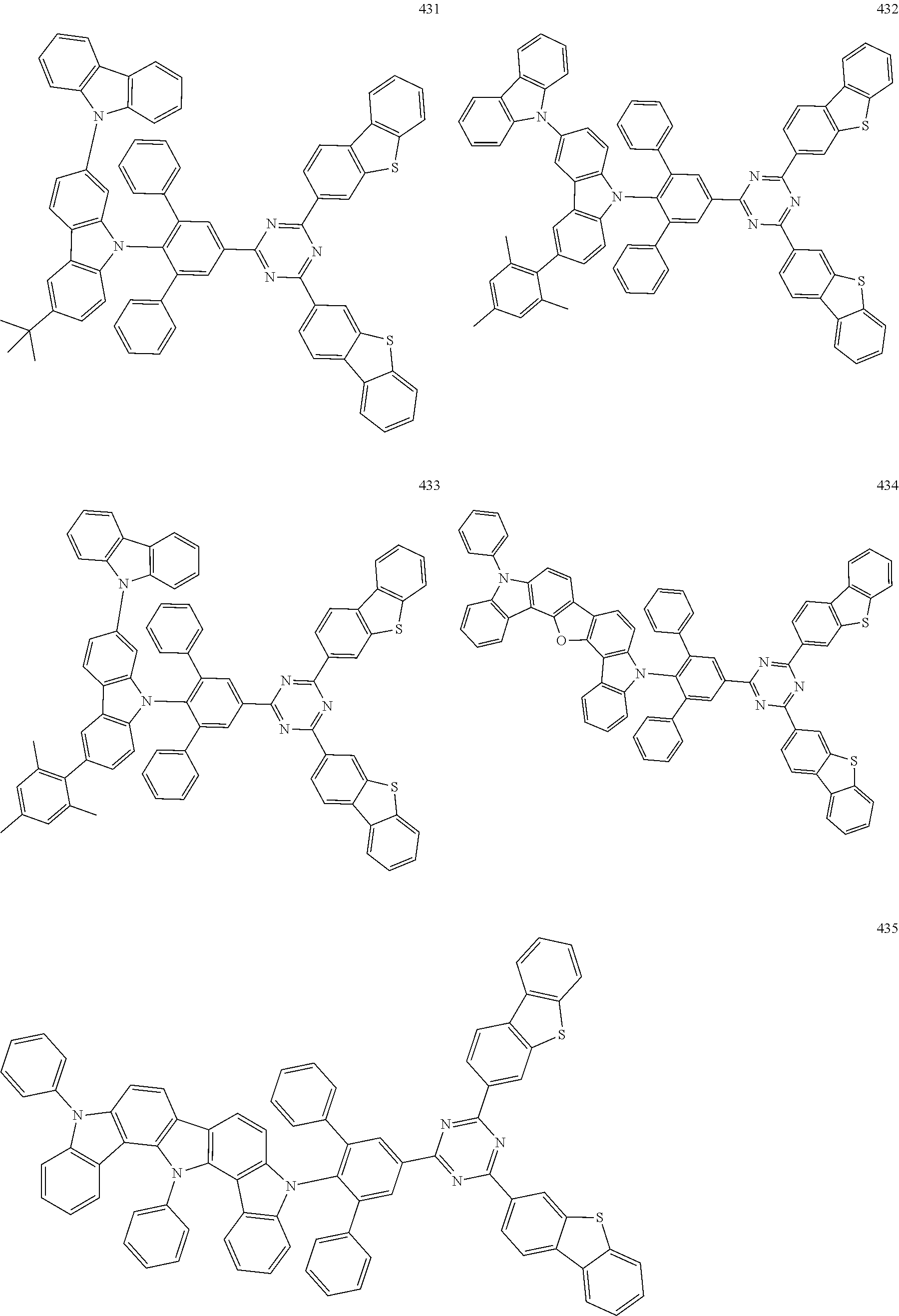

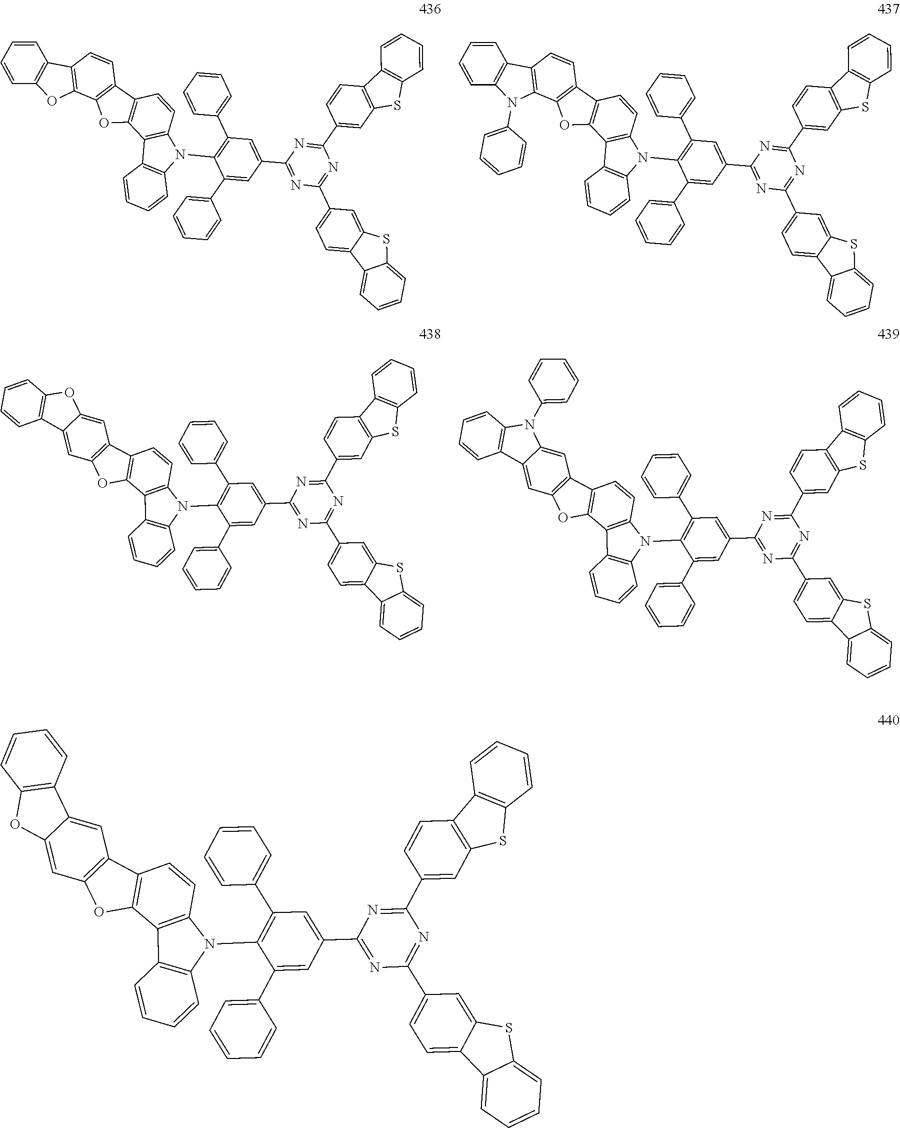

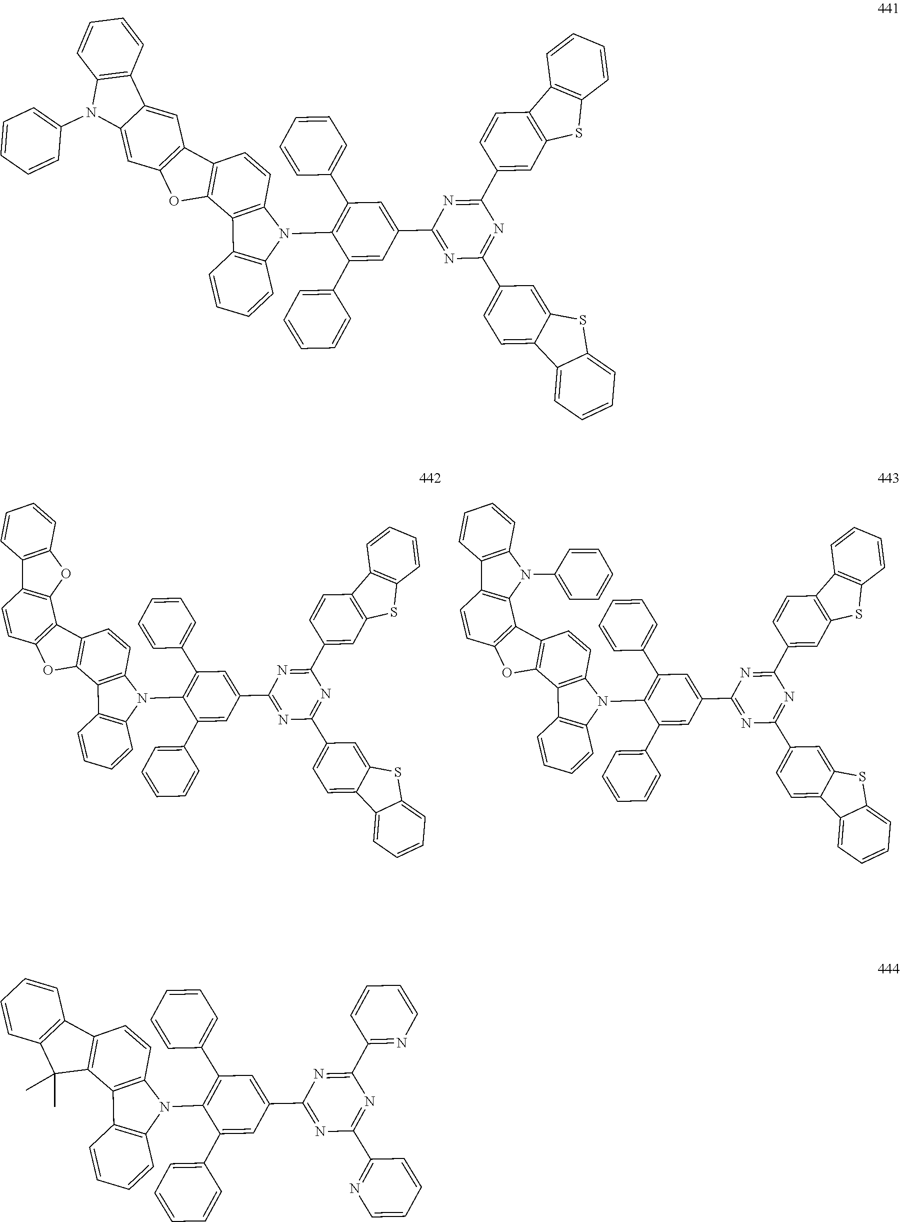

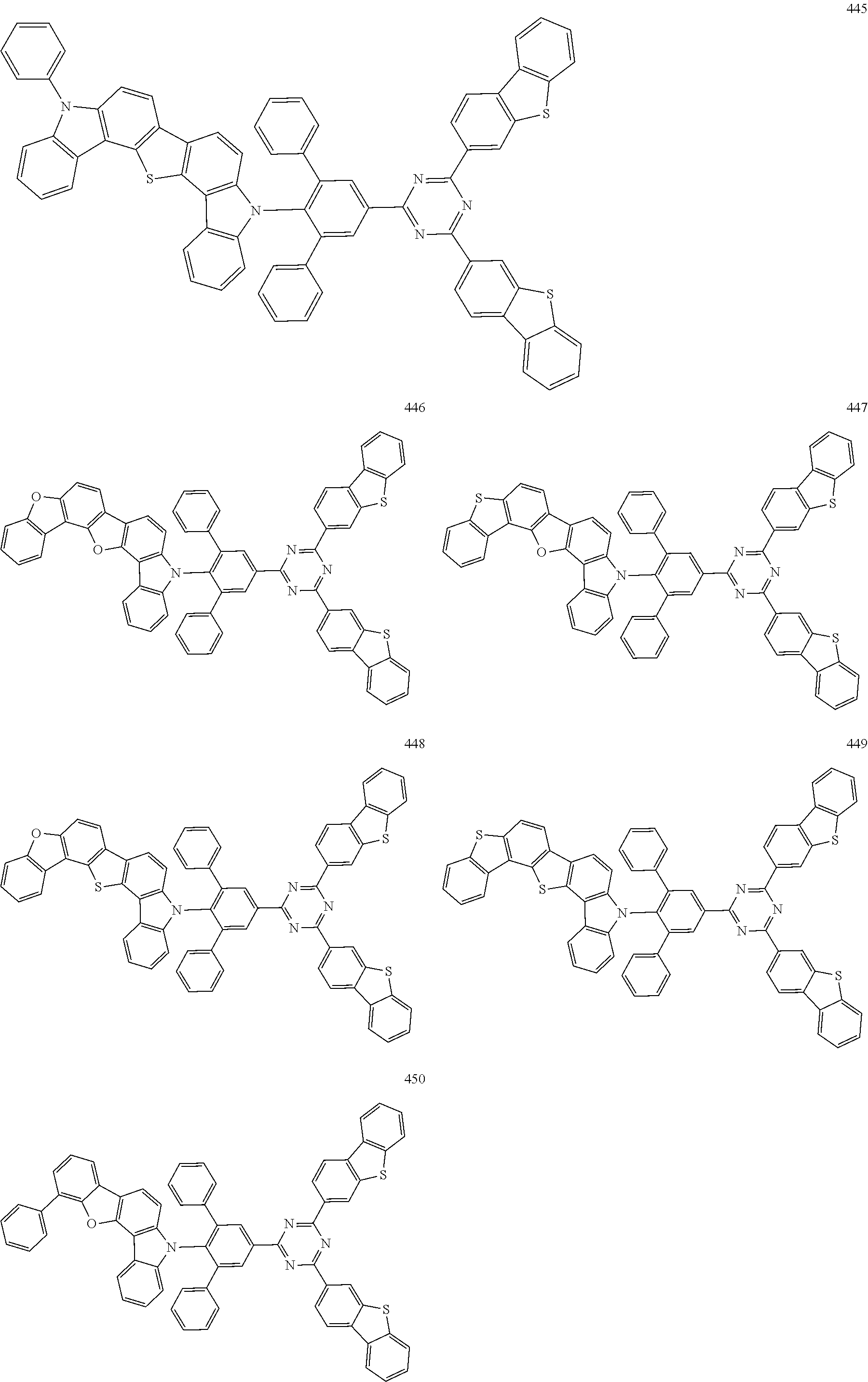

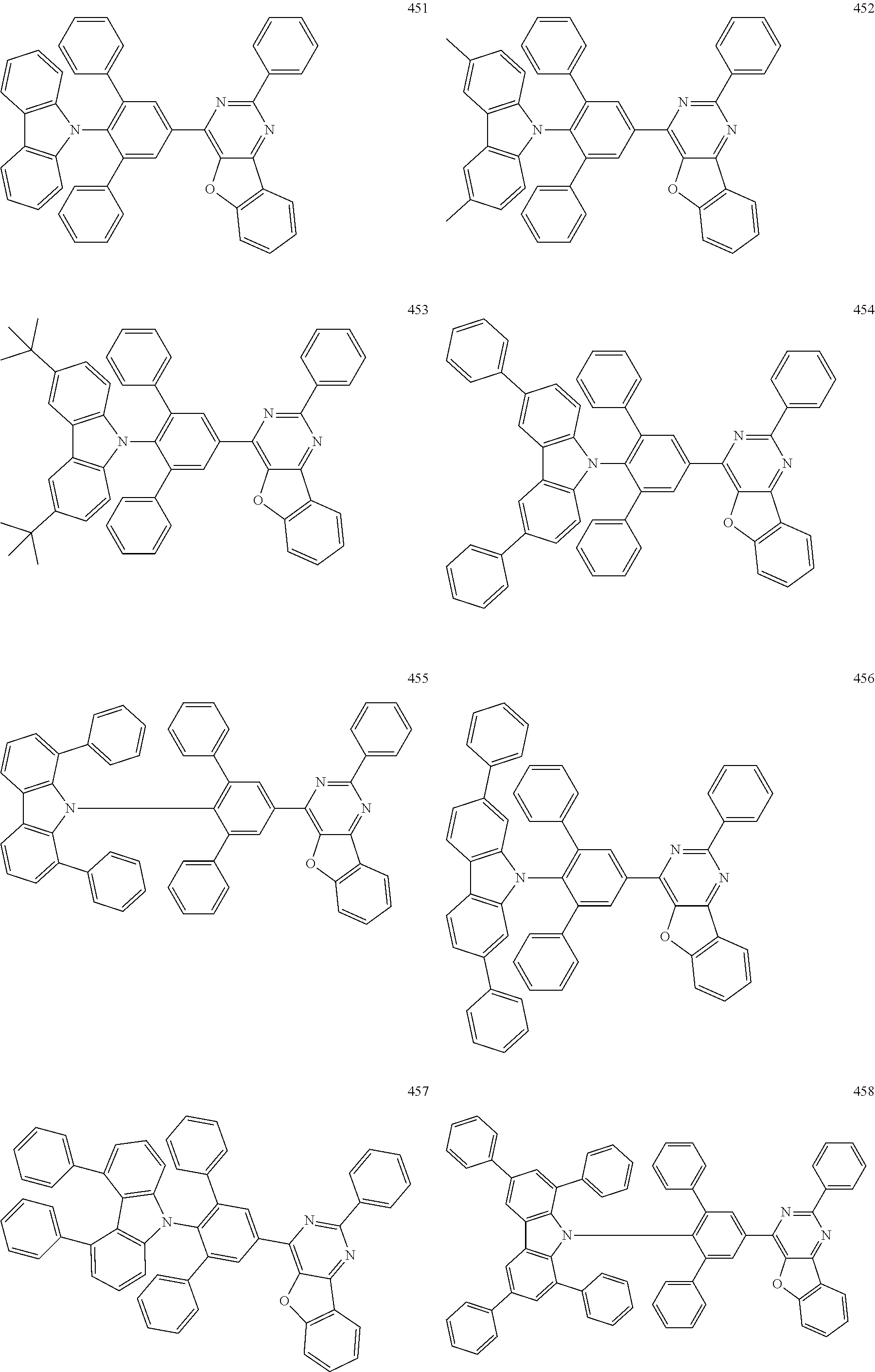

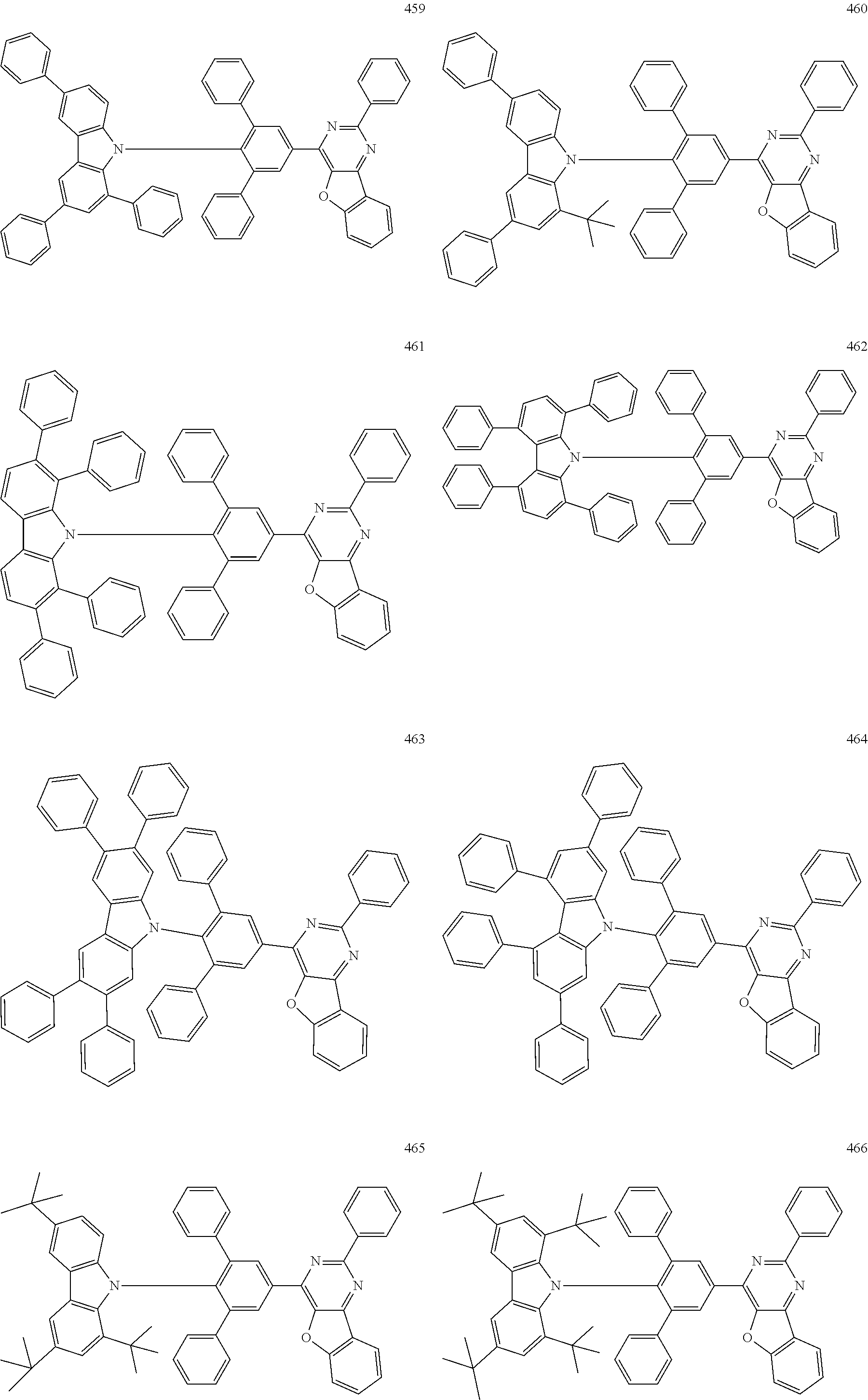

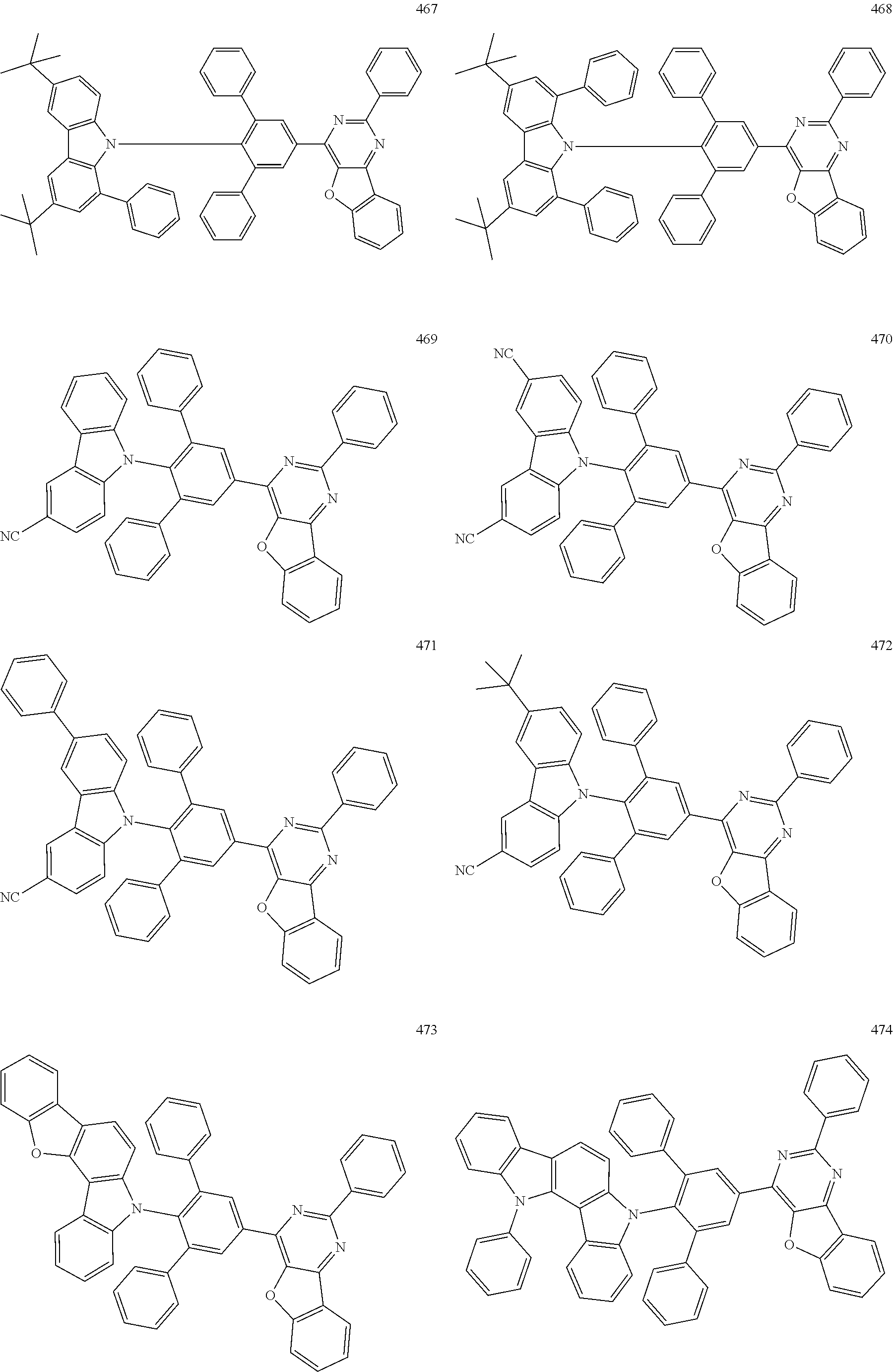

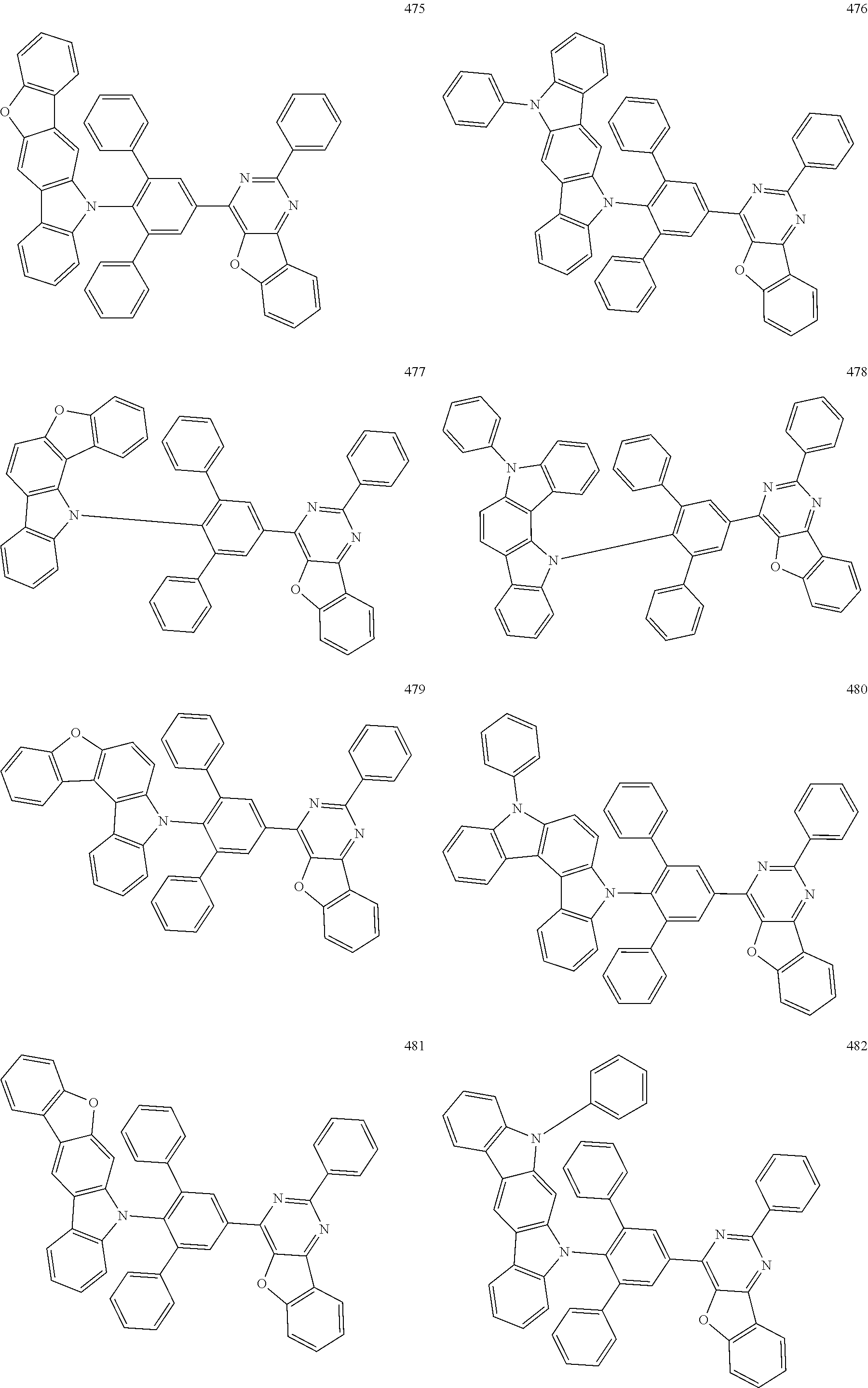

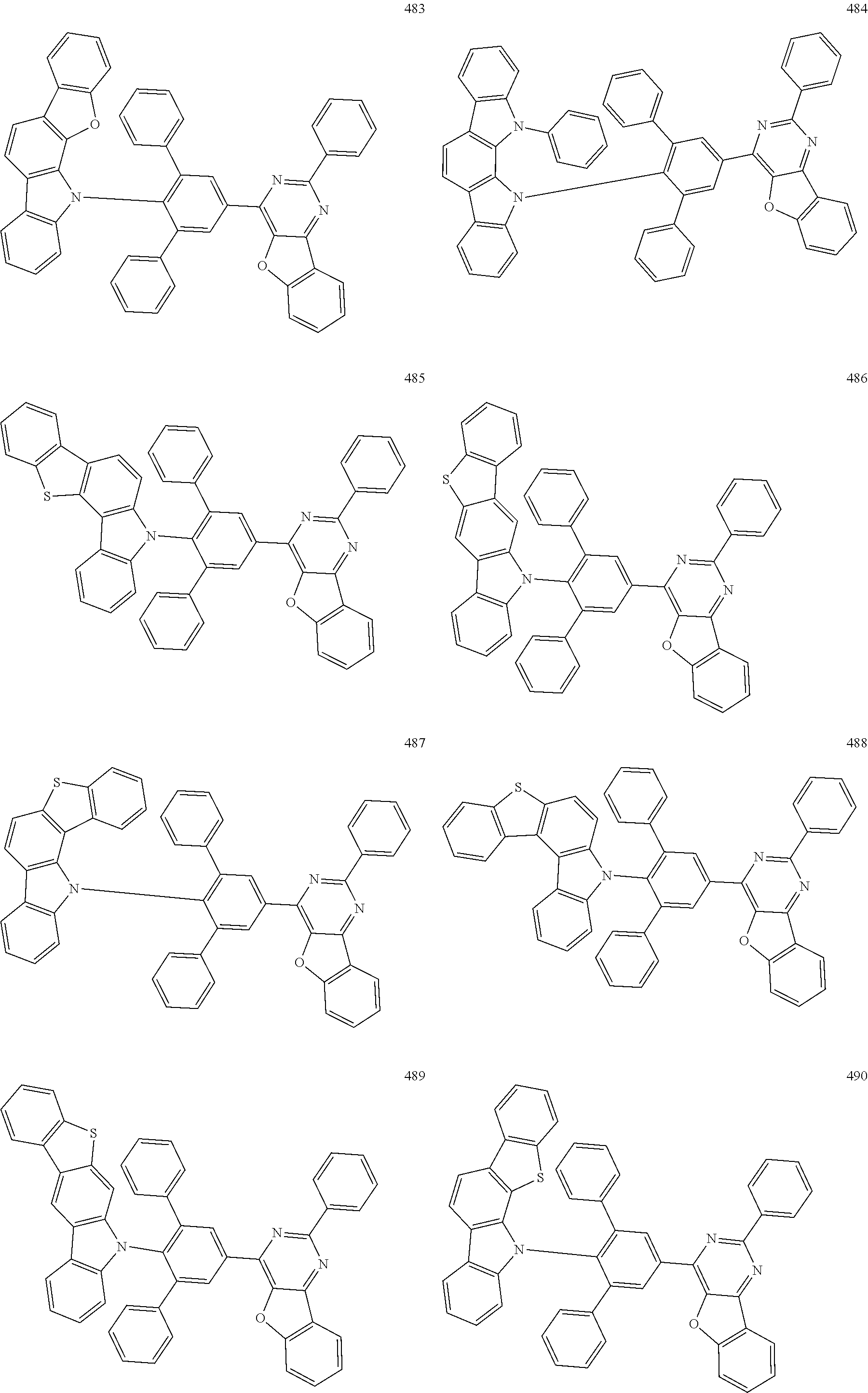

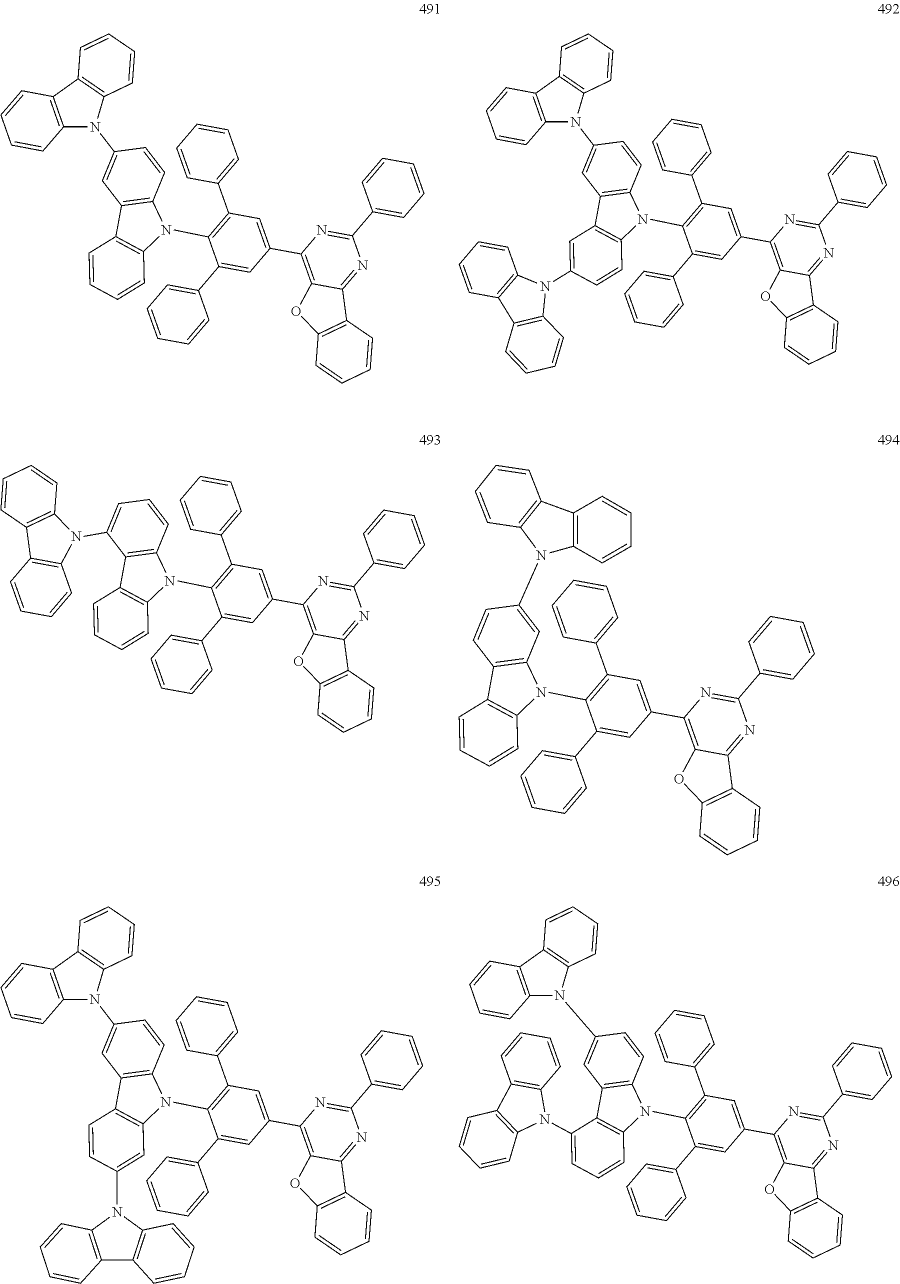

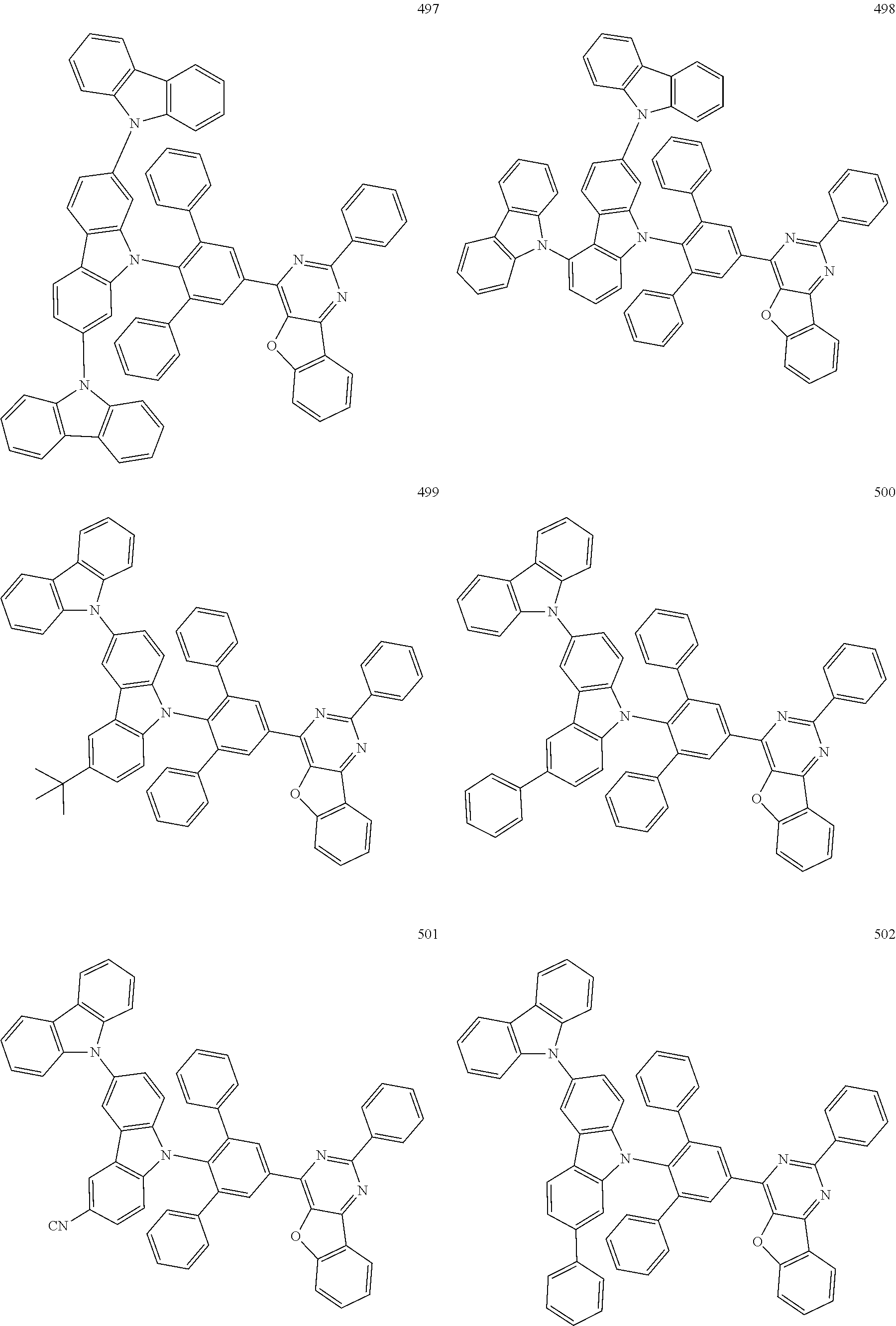

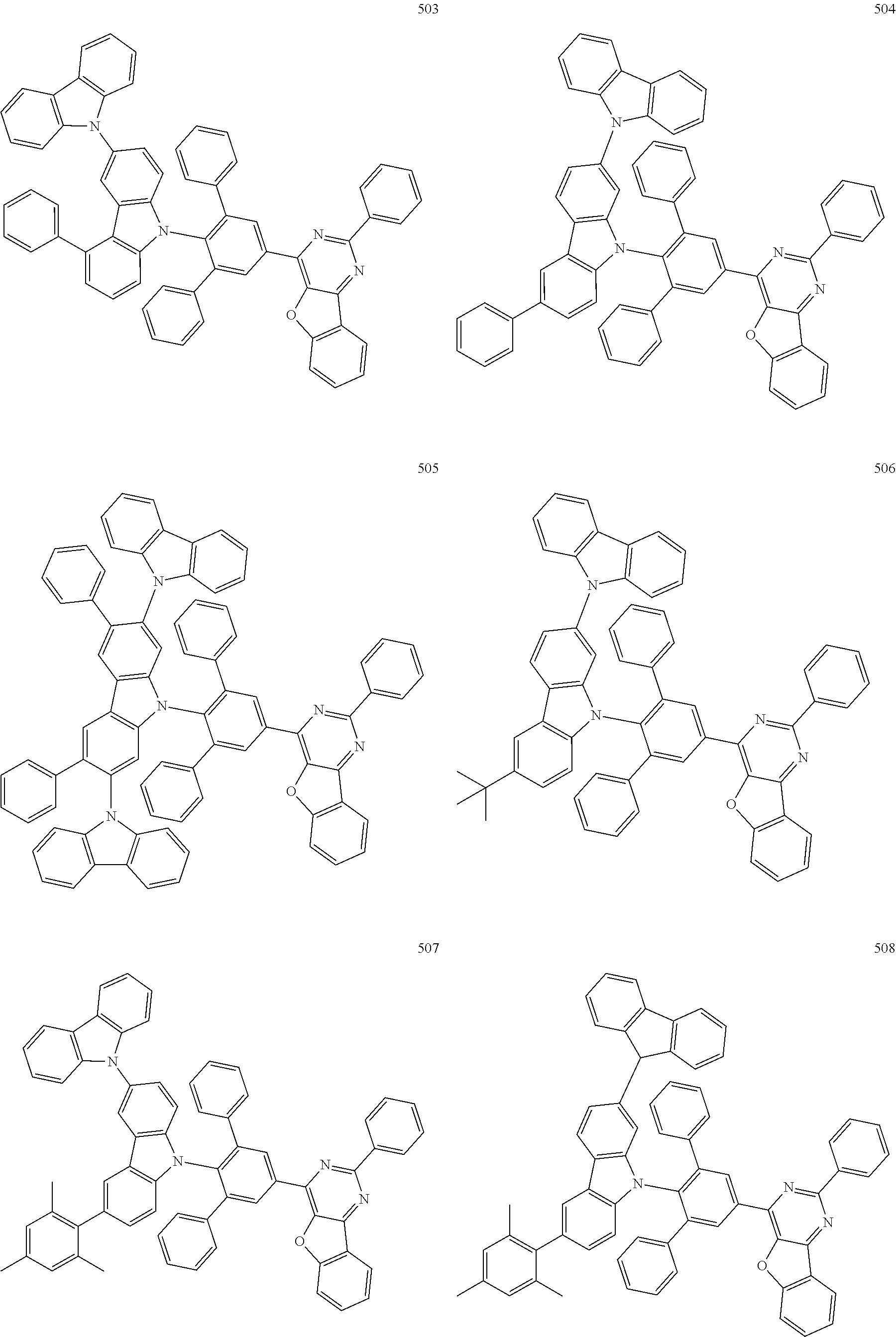

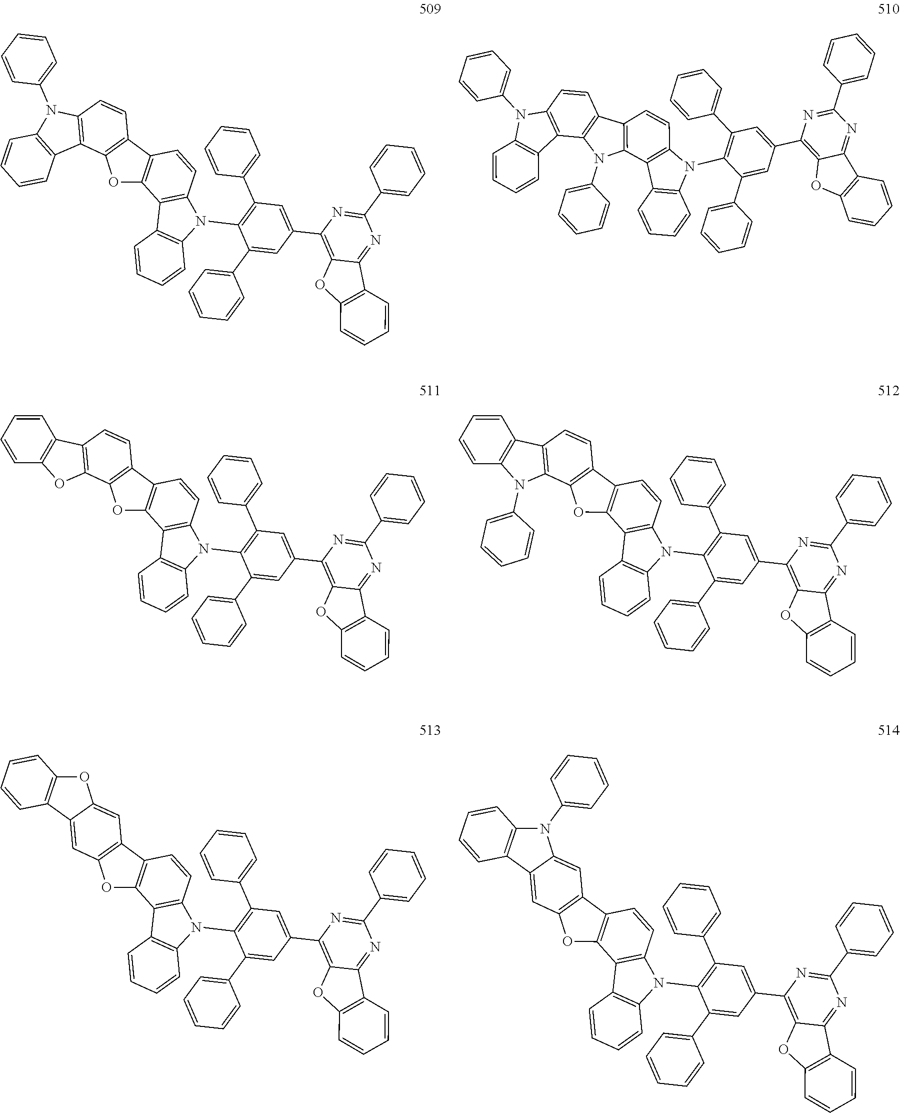

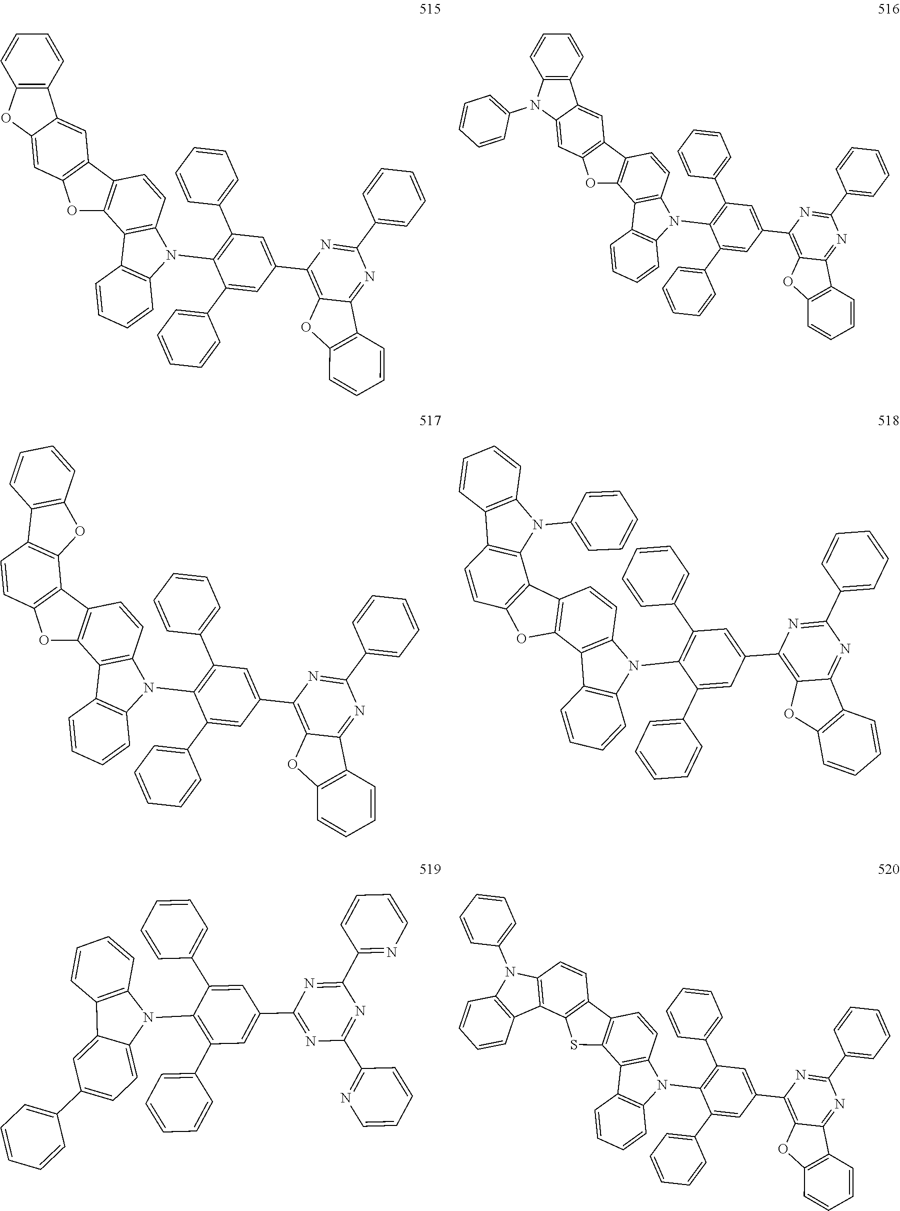

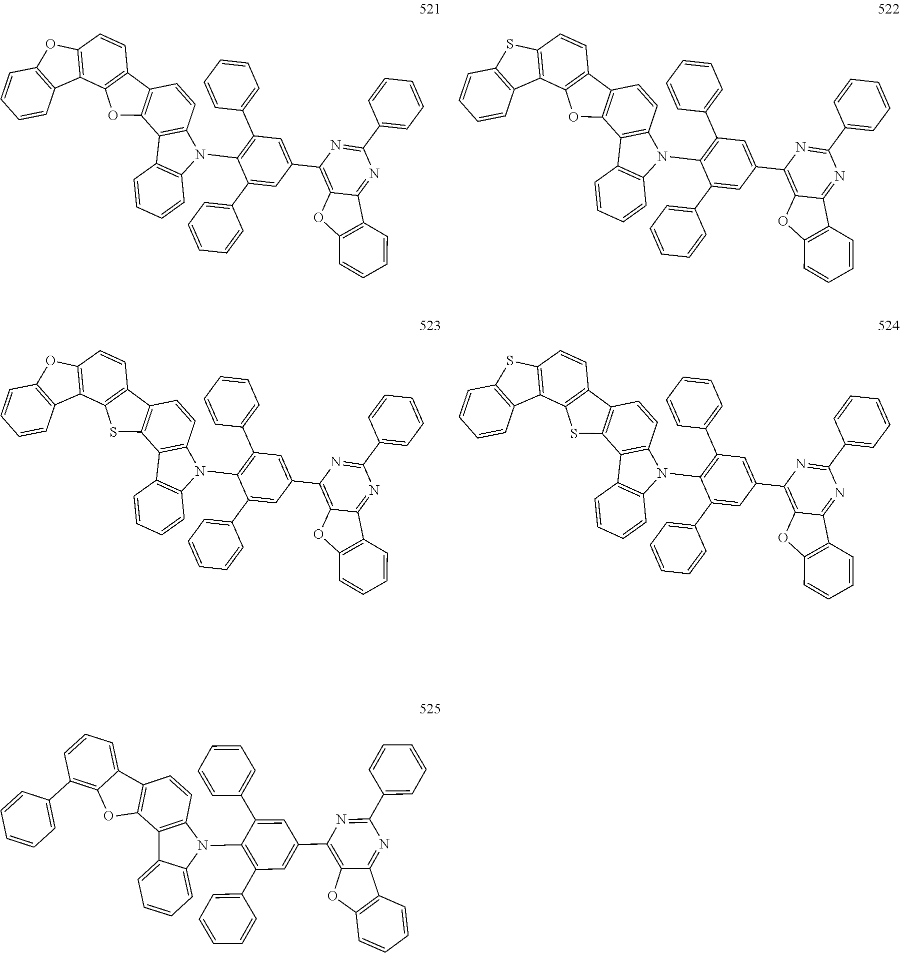

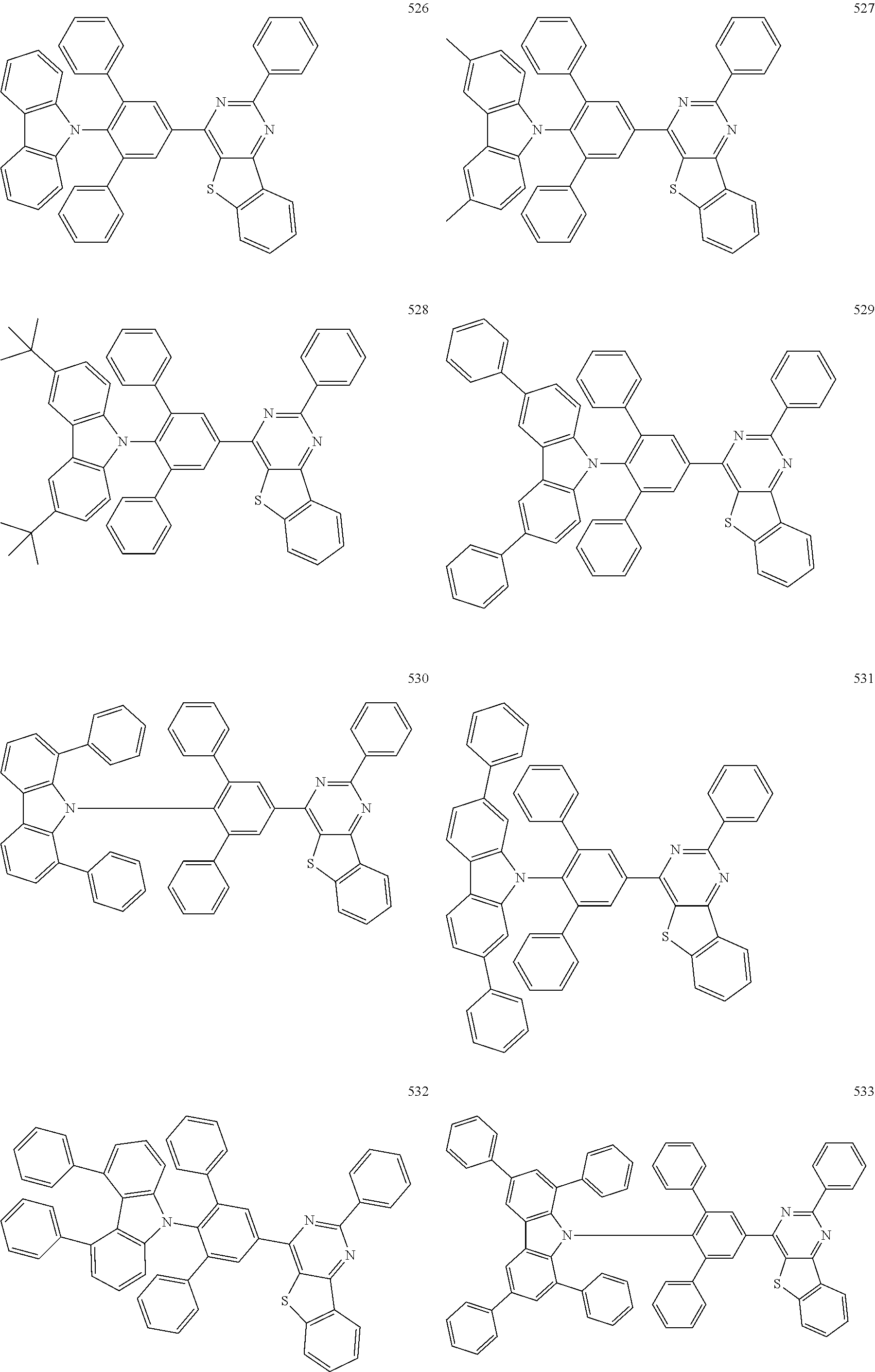

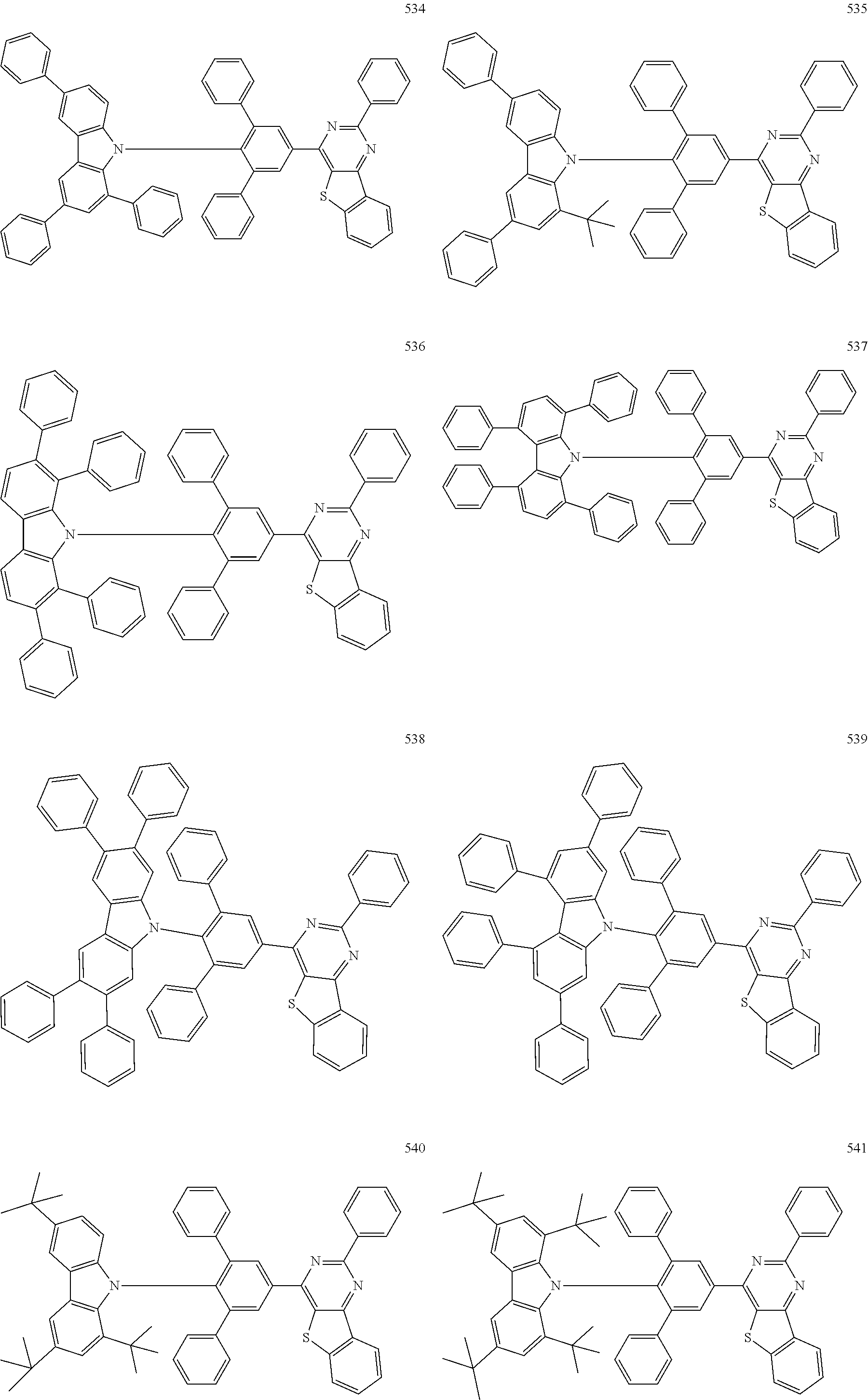

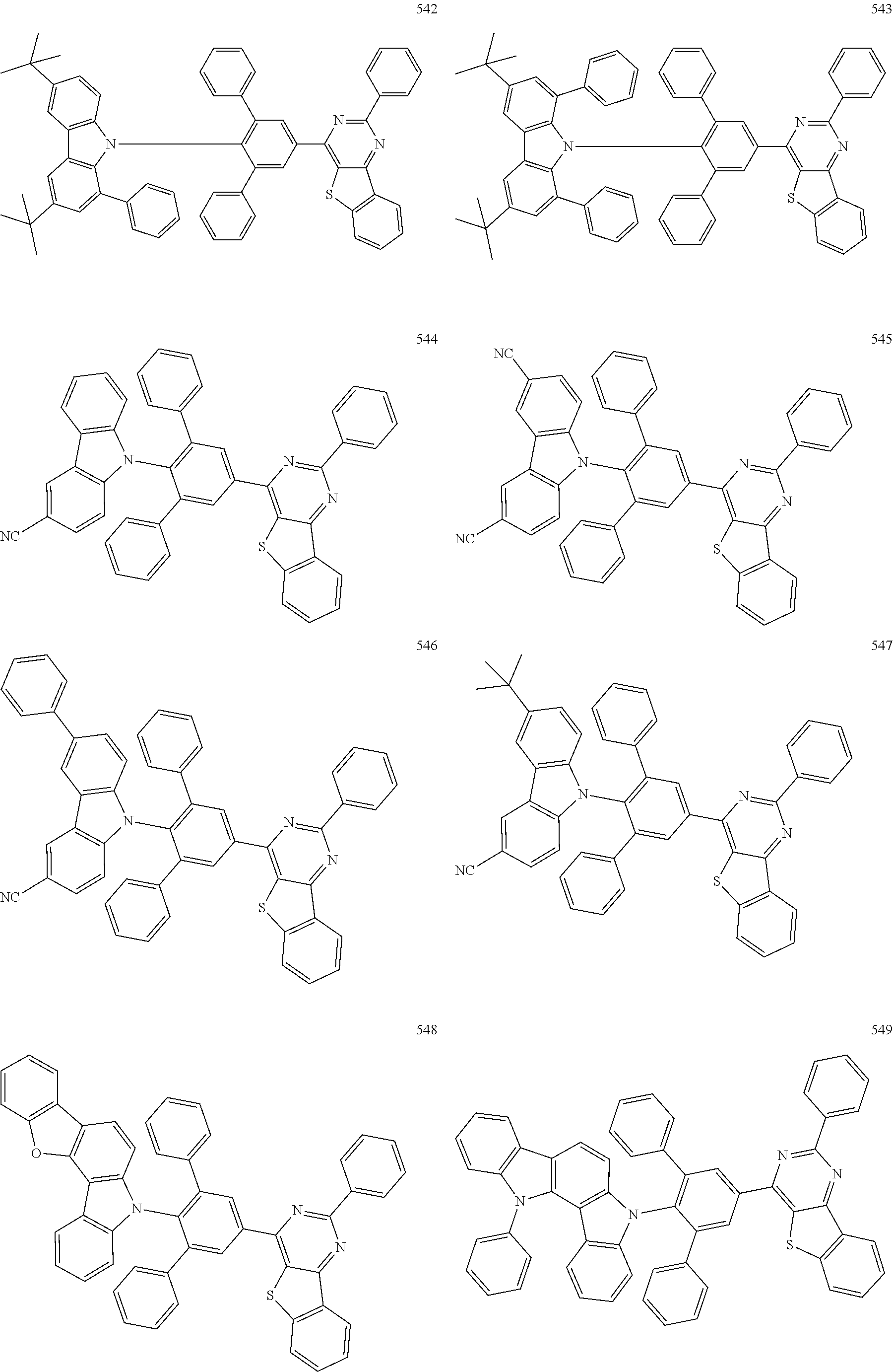

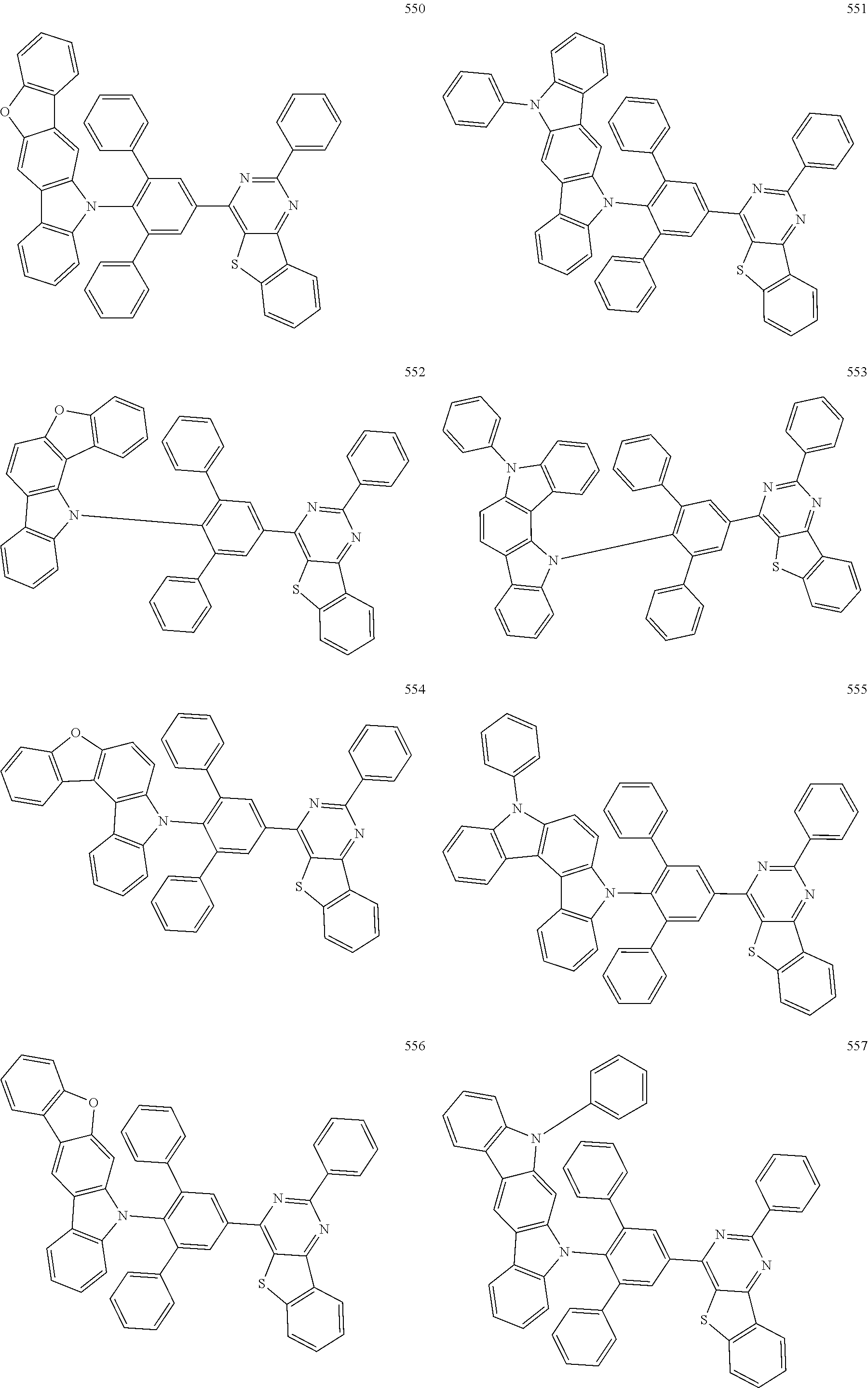

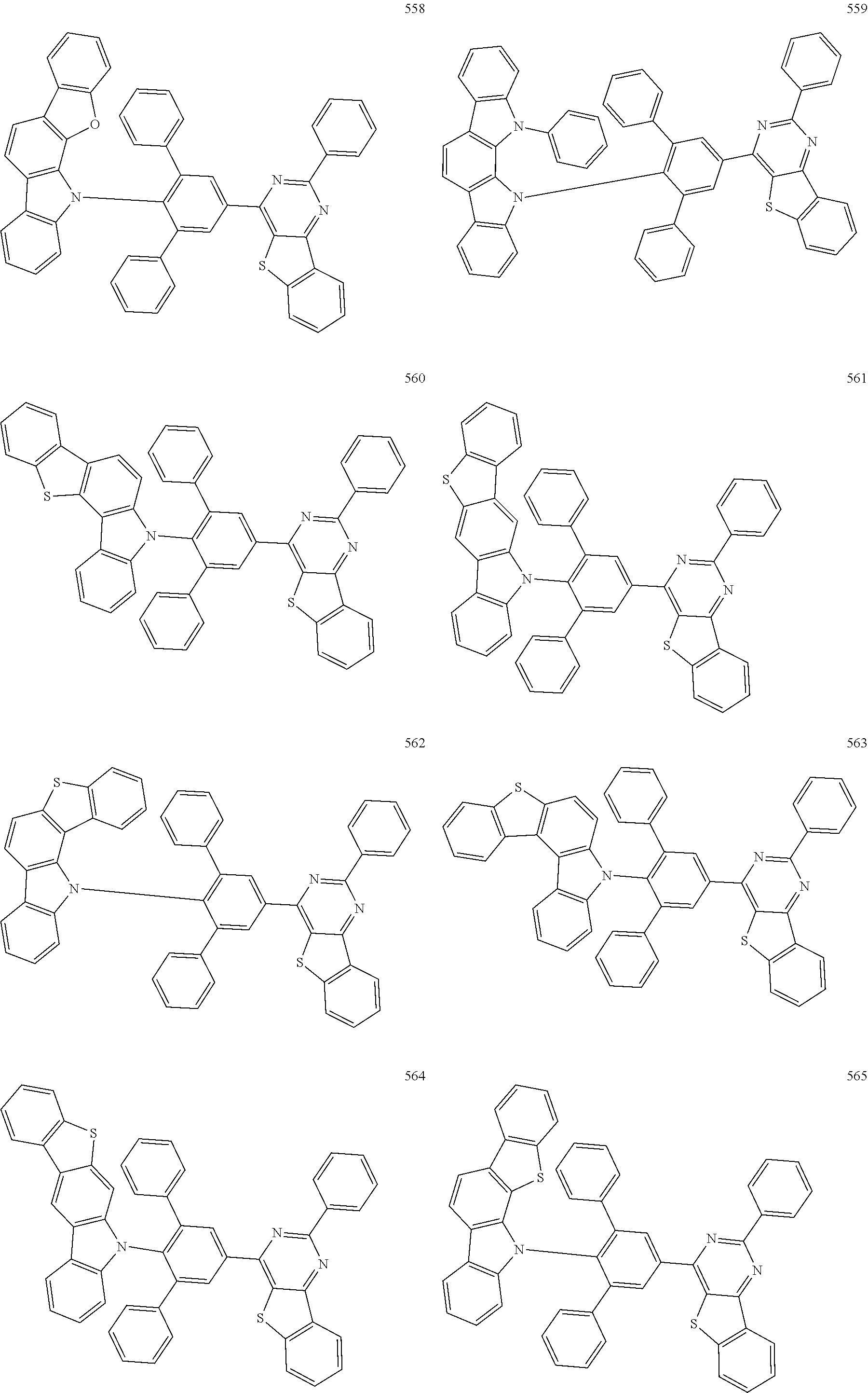

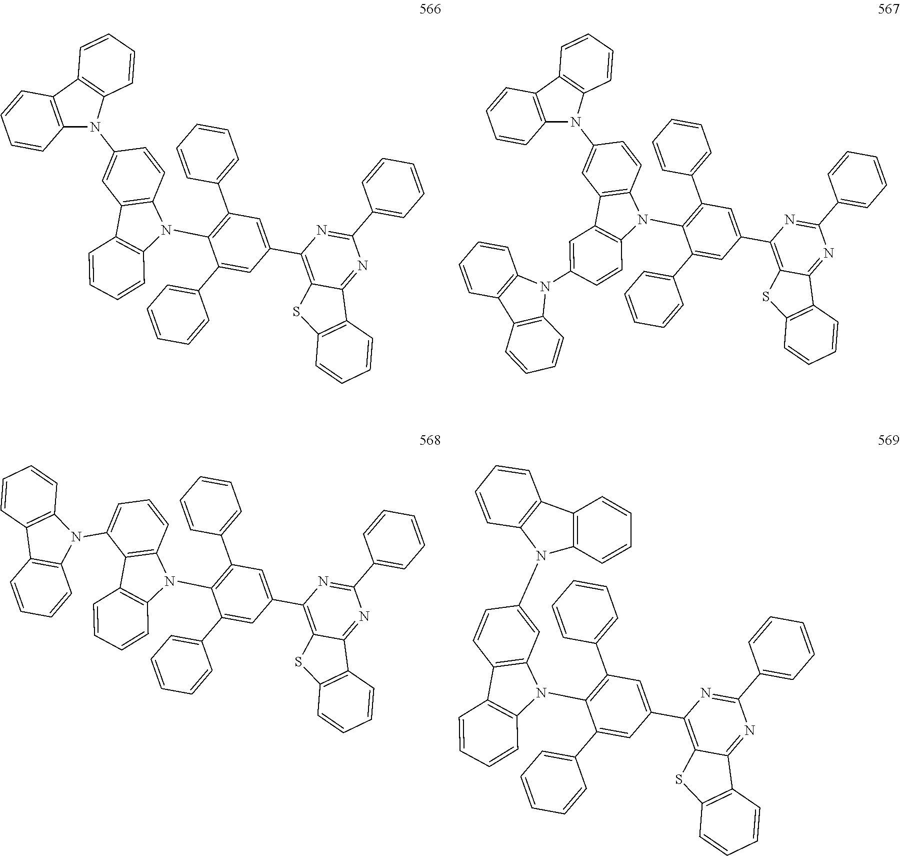 [SMILES: c1ccc(-c2nc(-c3cc(-c4ccccc4)c(-n4c5ccccc5c5cc(-n6c7ccccc7c7ccccc76)ccc54)c(-c4ccccc4)c3)c3sc4ccccc4c3n2)cc1 ; c1ccc(-c2nc(-c3cc(-c4ccccc4)c(-n4c5ccc(-n6c7ccccc7c7ccccc76)cc5c5cc(-n6c7ccccc7c7ccccc76)ccc54)c(-c4ccccc4)c3)c3sc4ccccc4c3n2)cc1 ; c1ccc(-c2nc(-c3cc(-c4ccccc4)c(-n4c5ccccc5c5c(-n6c7ccccc7c7ccccc76)cccc54)c(-c4ccccc4)c3)c3sc4ccccc4c3n2)cc1 ; c1ccc(-c2nc(-c3cc(-c4ccccc4)c(-n4c5ccccc5c5ccc(-n6c7ccccc7c7ccccc76)cc54)c(-c4ccccc4)c3)c3sc4ccccc4c3n2)cc1]

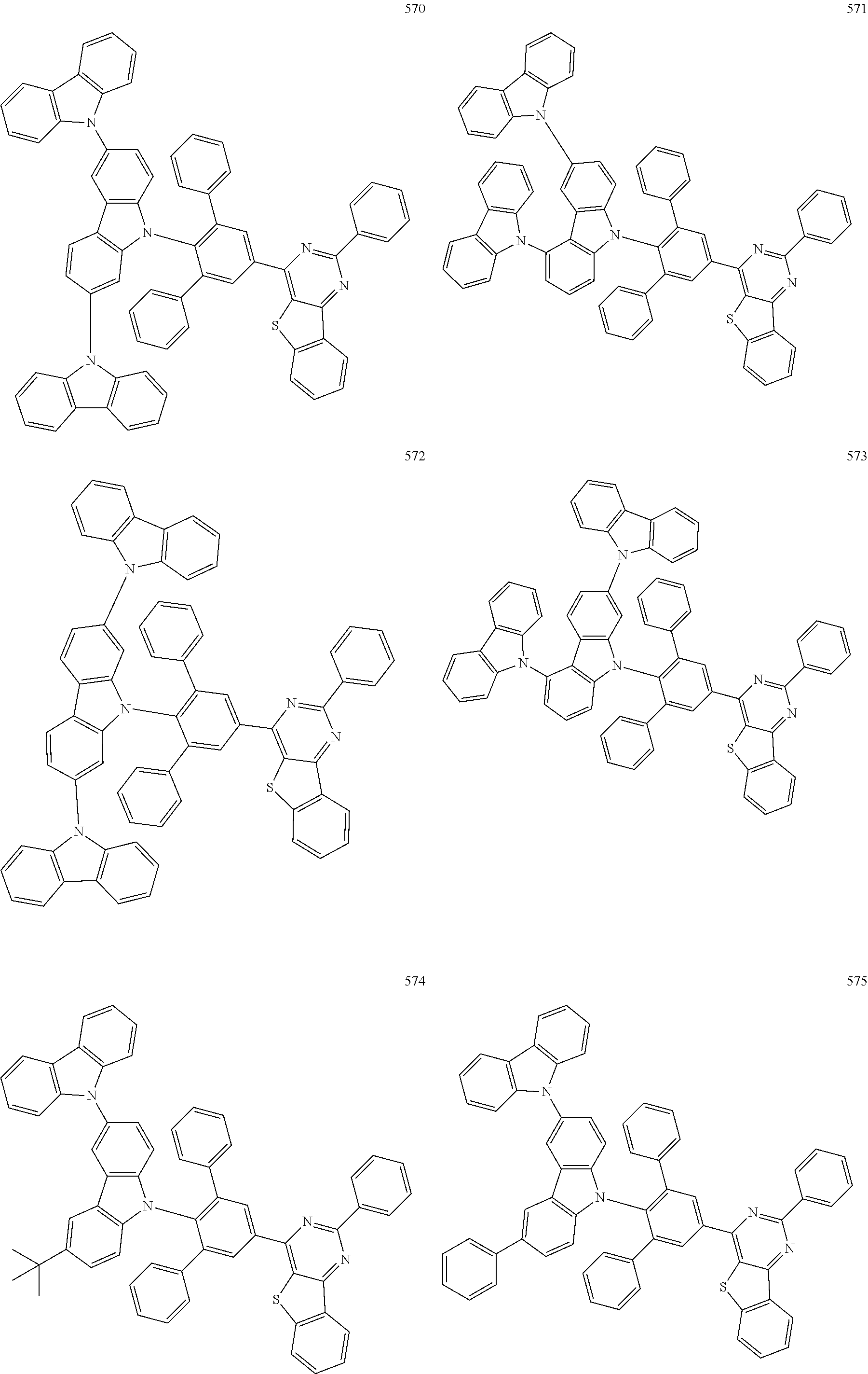

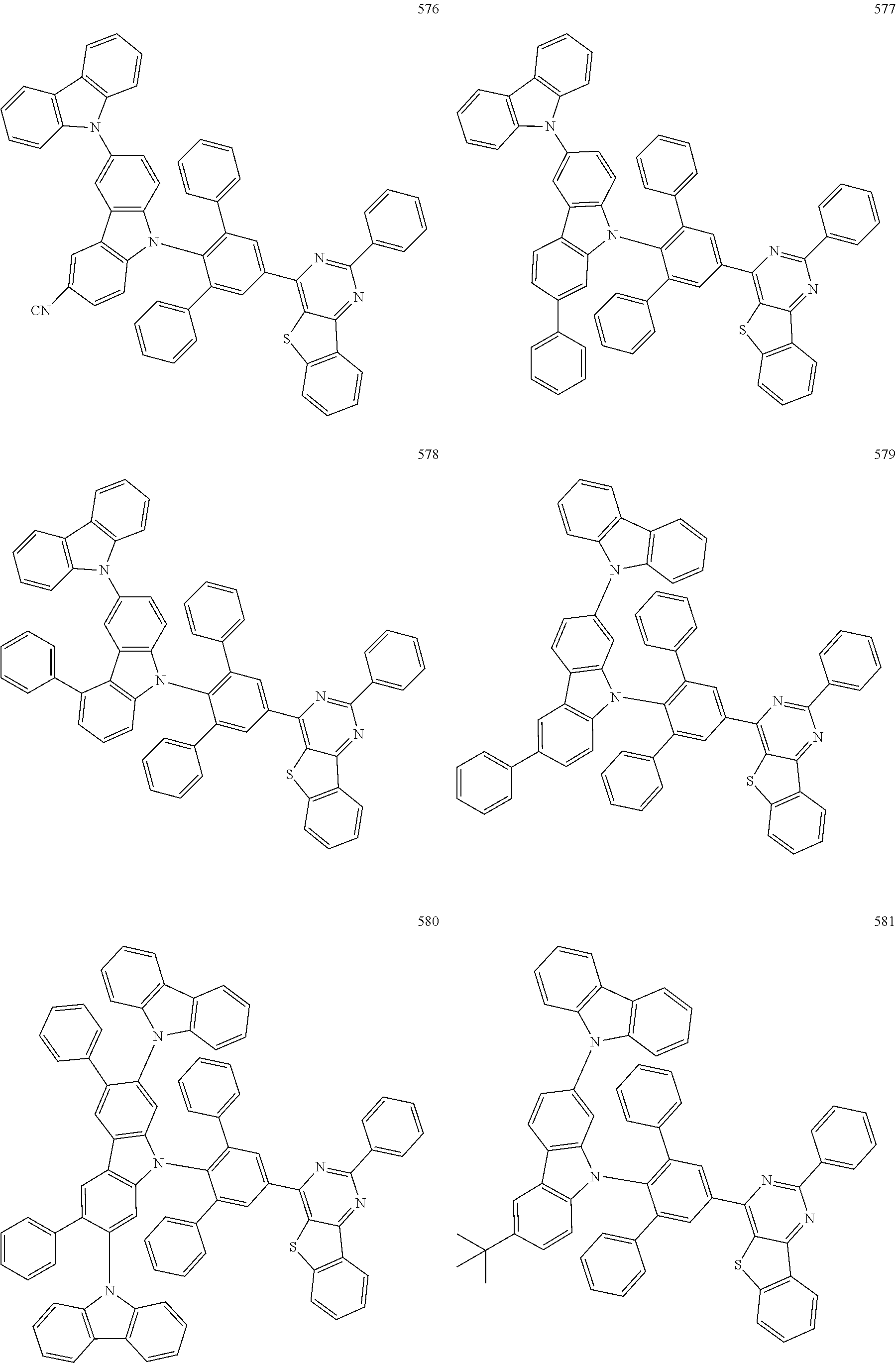

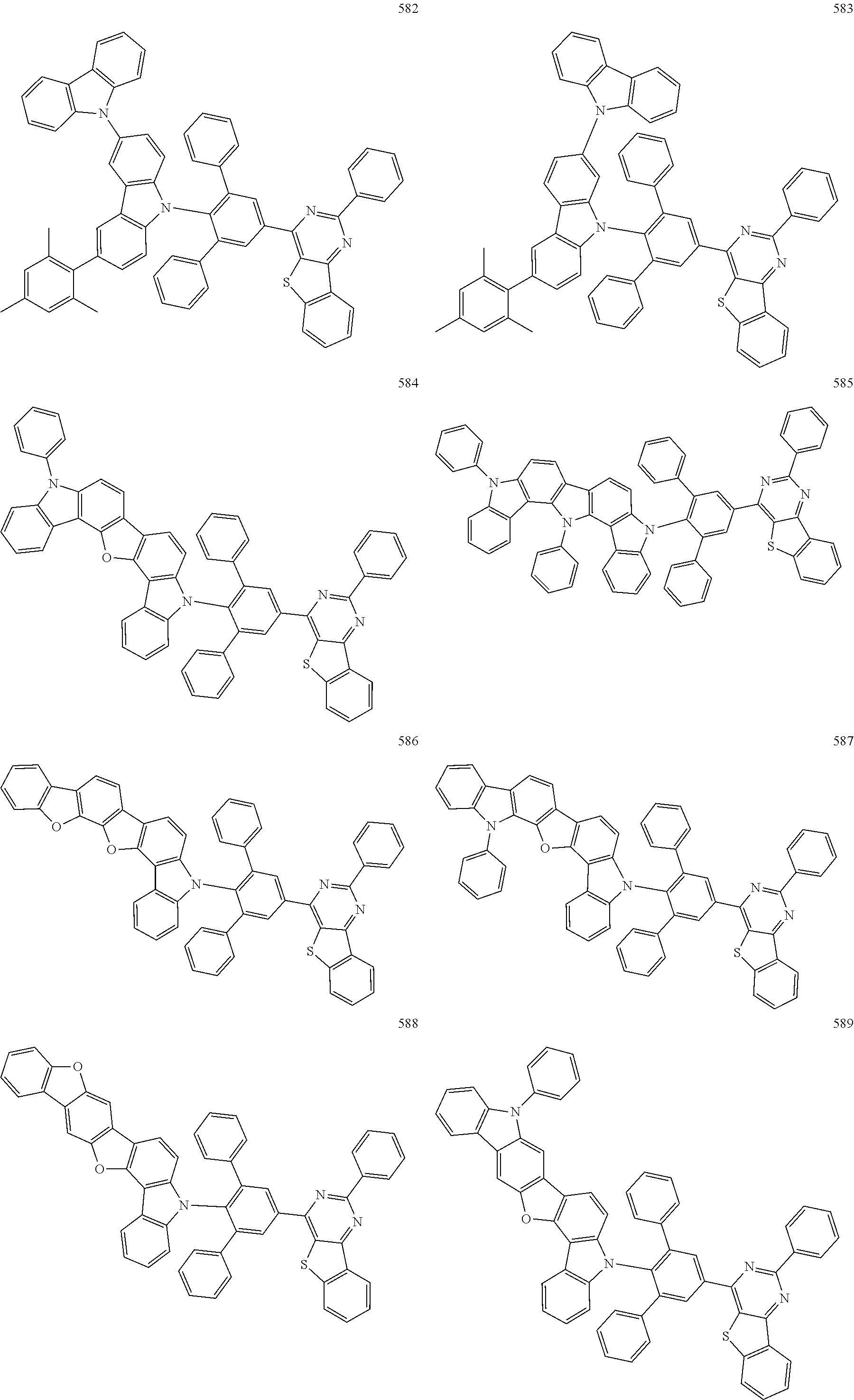

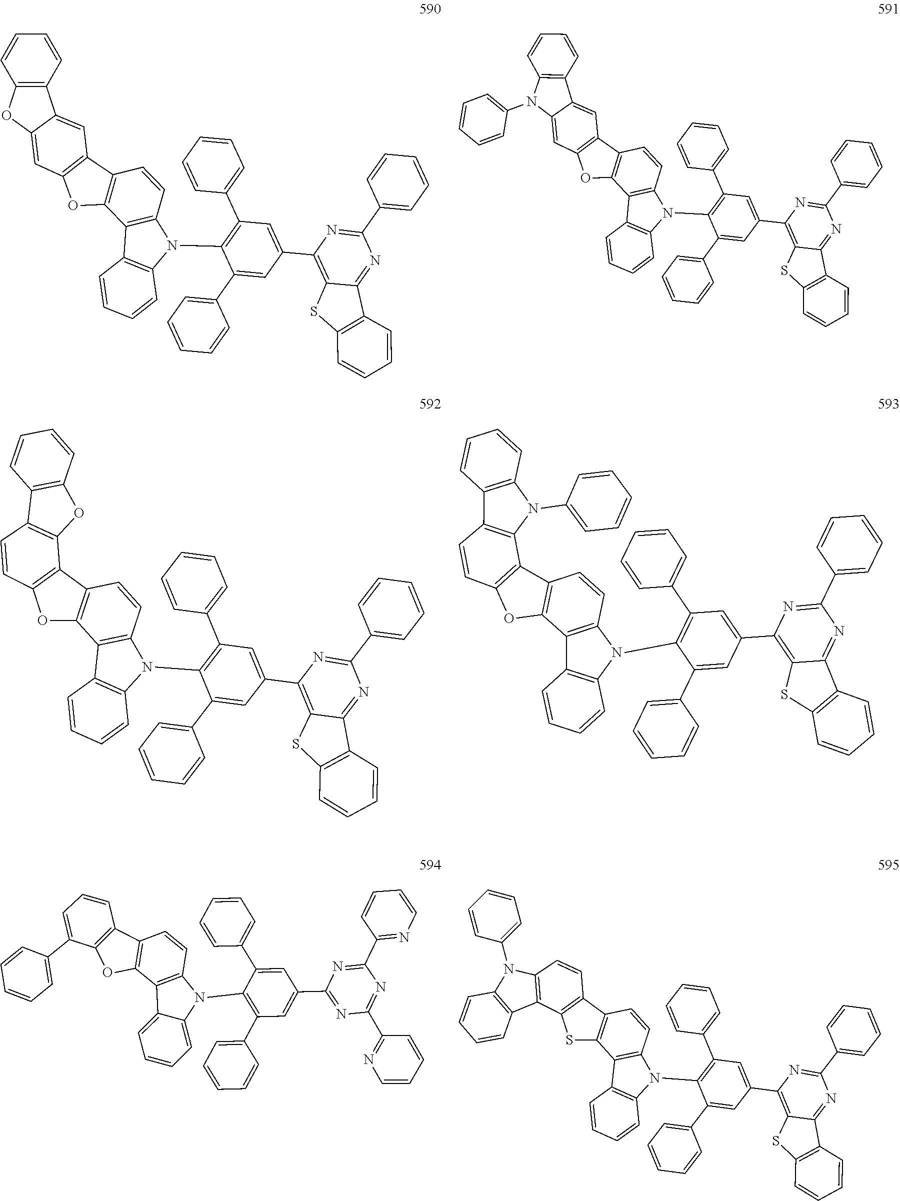

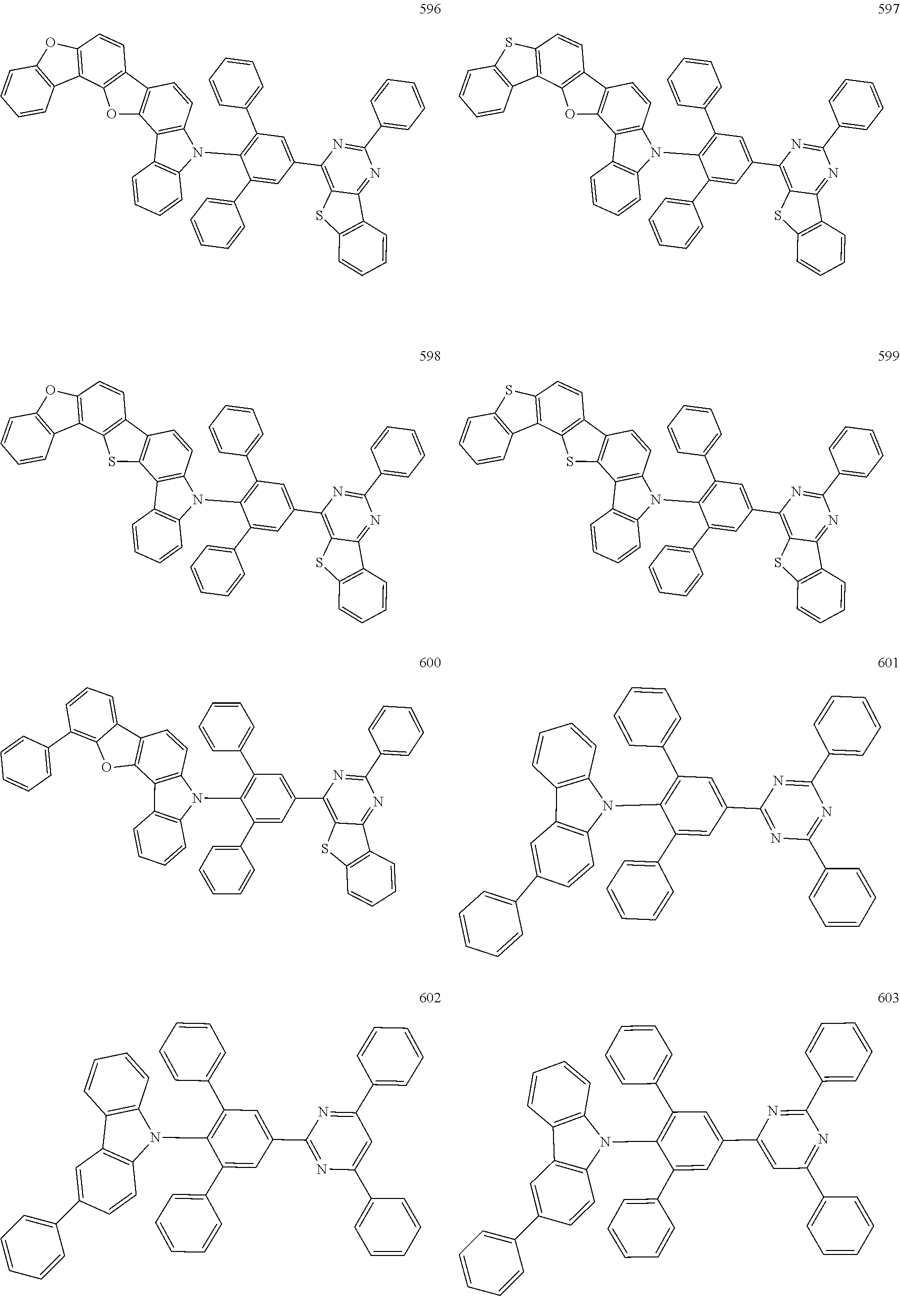

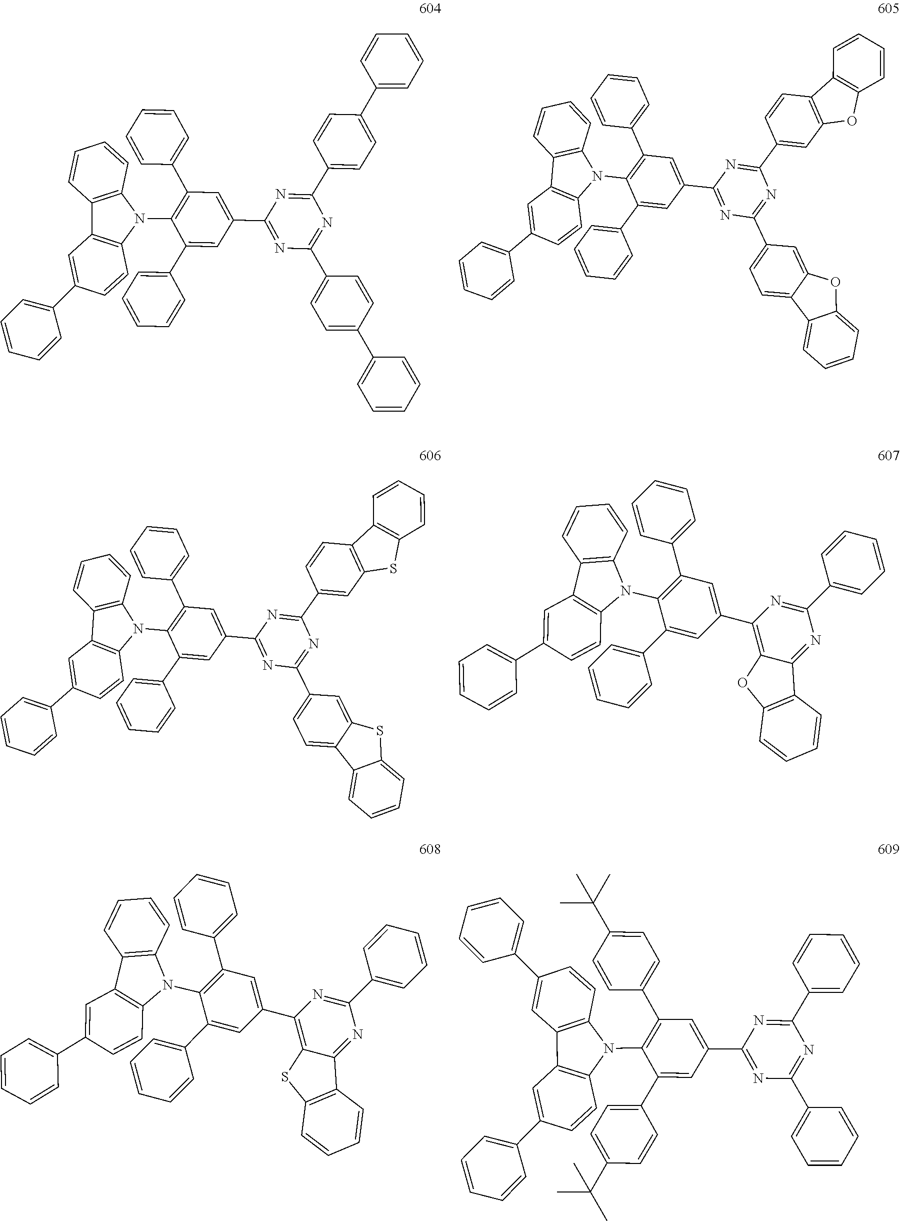

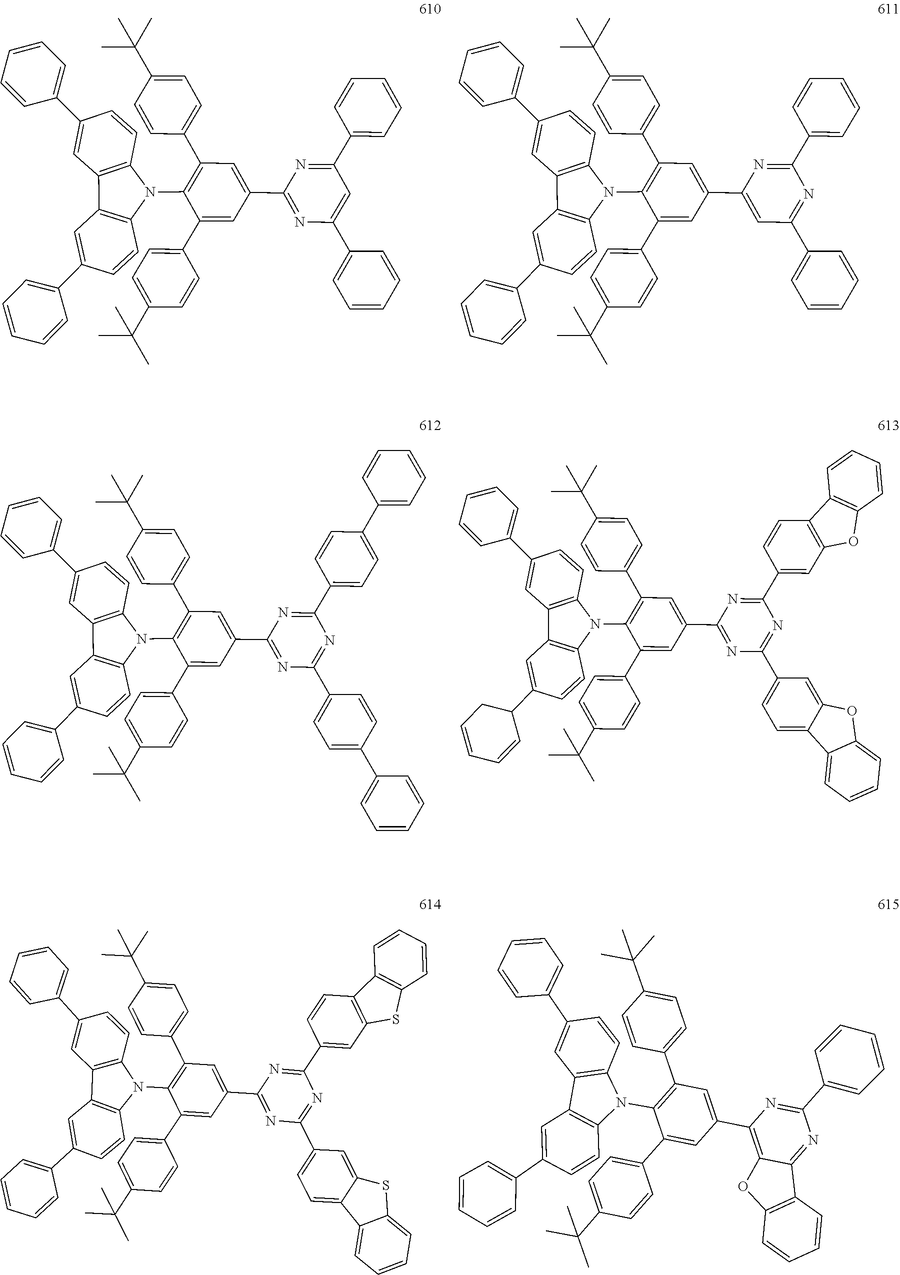

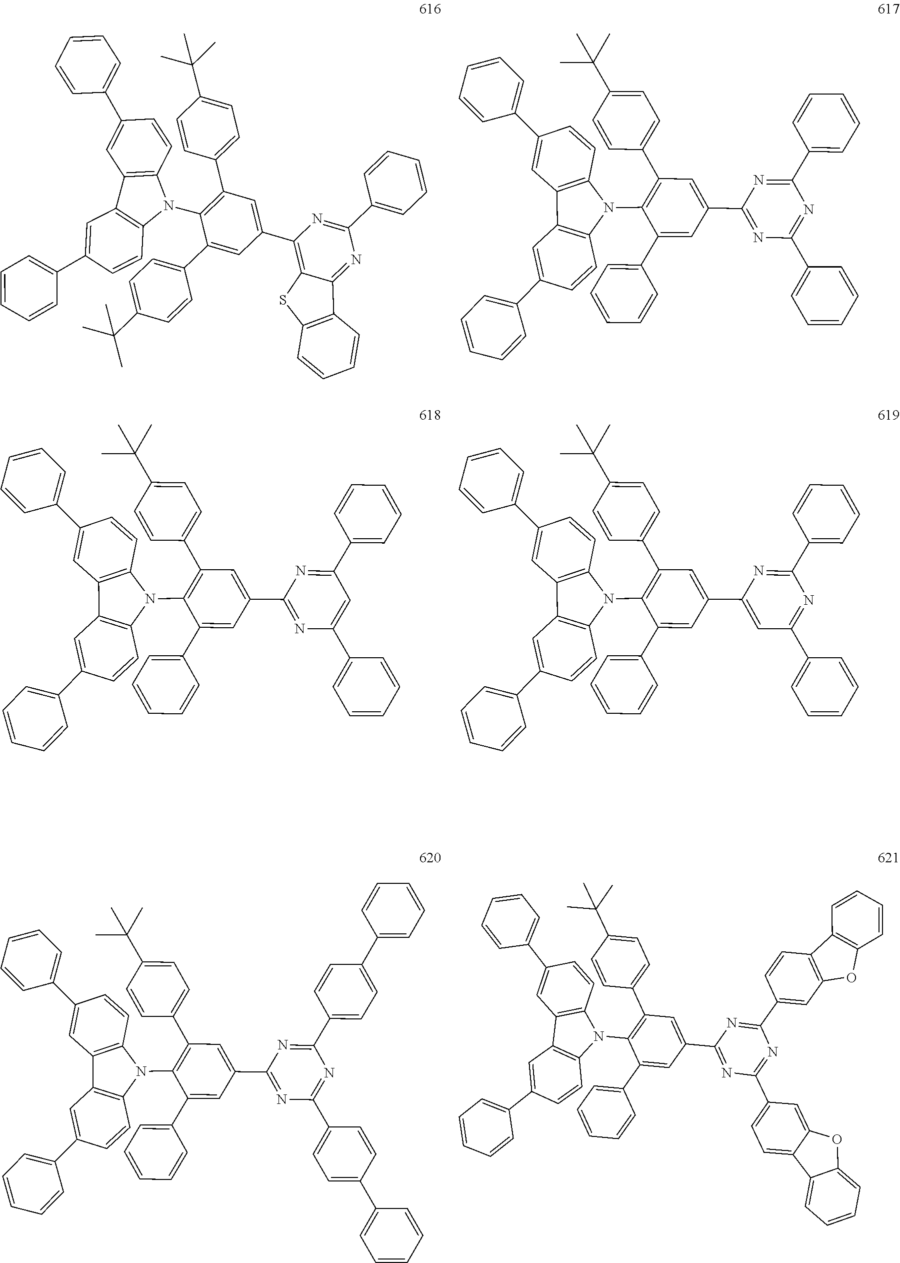

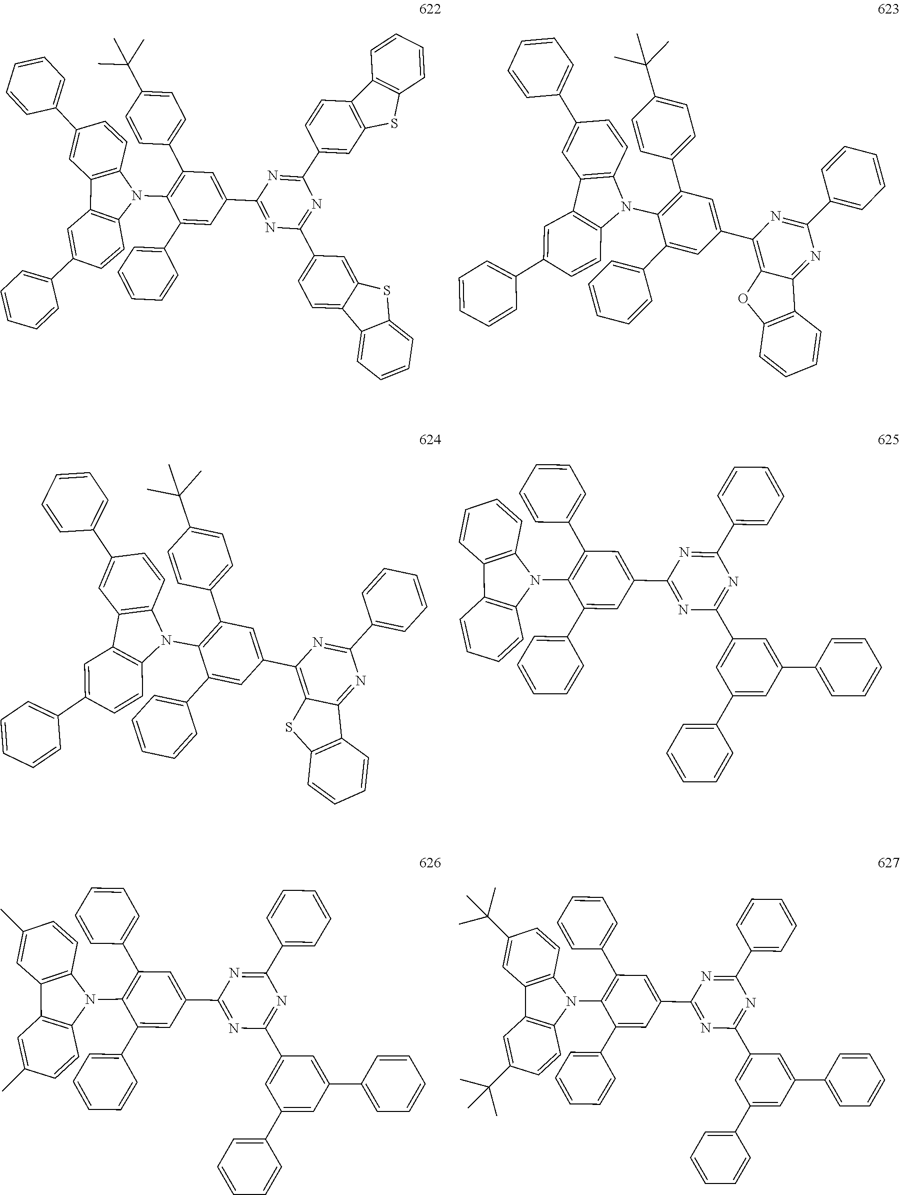

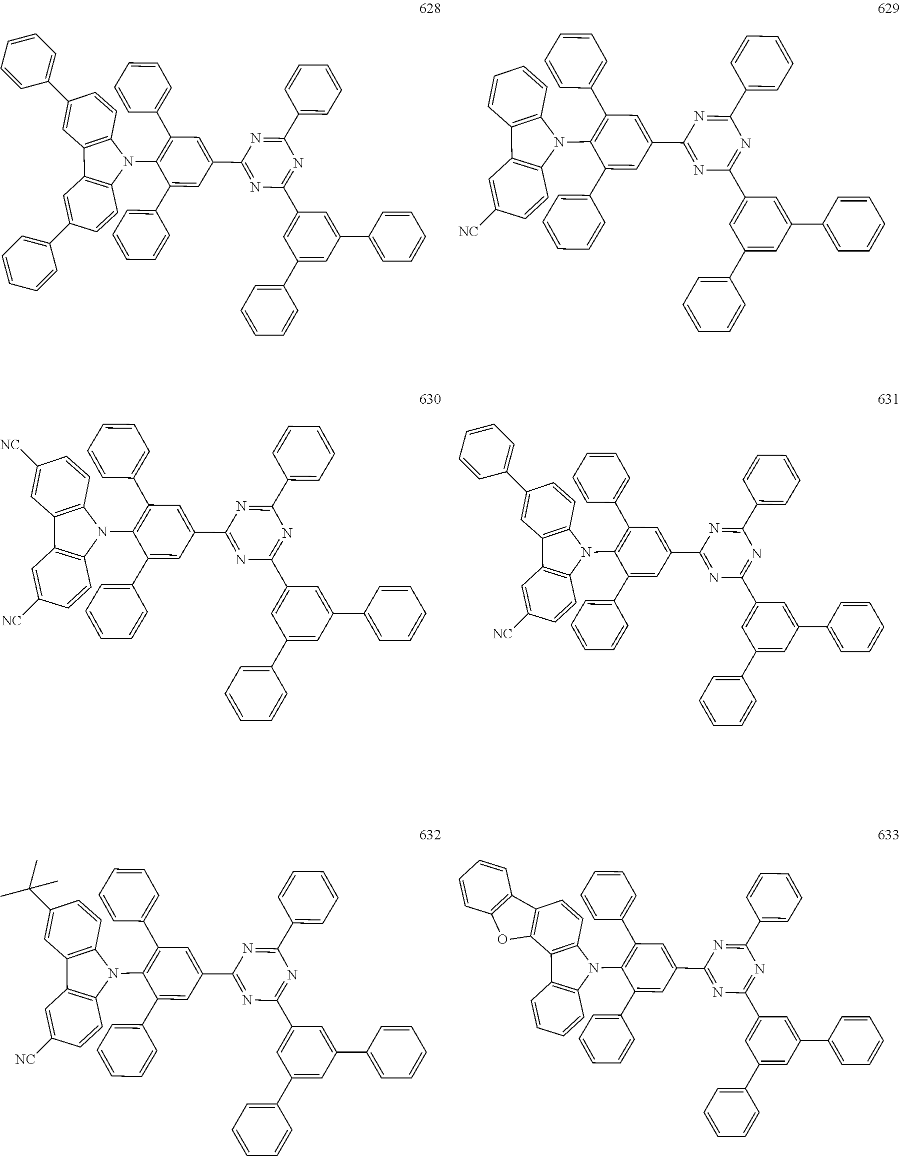

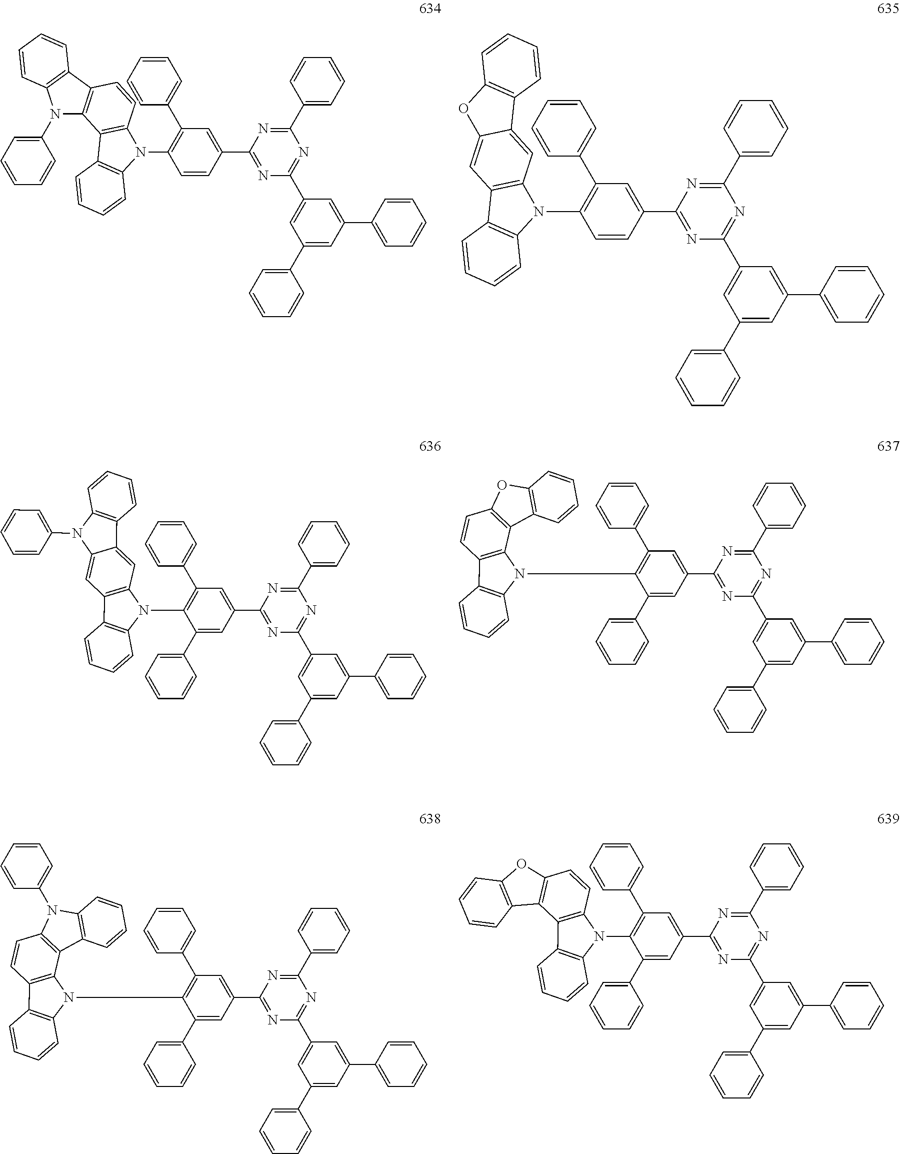

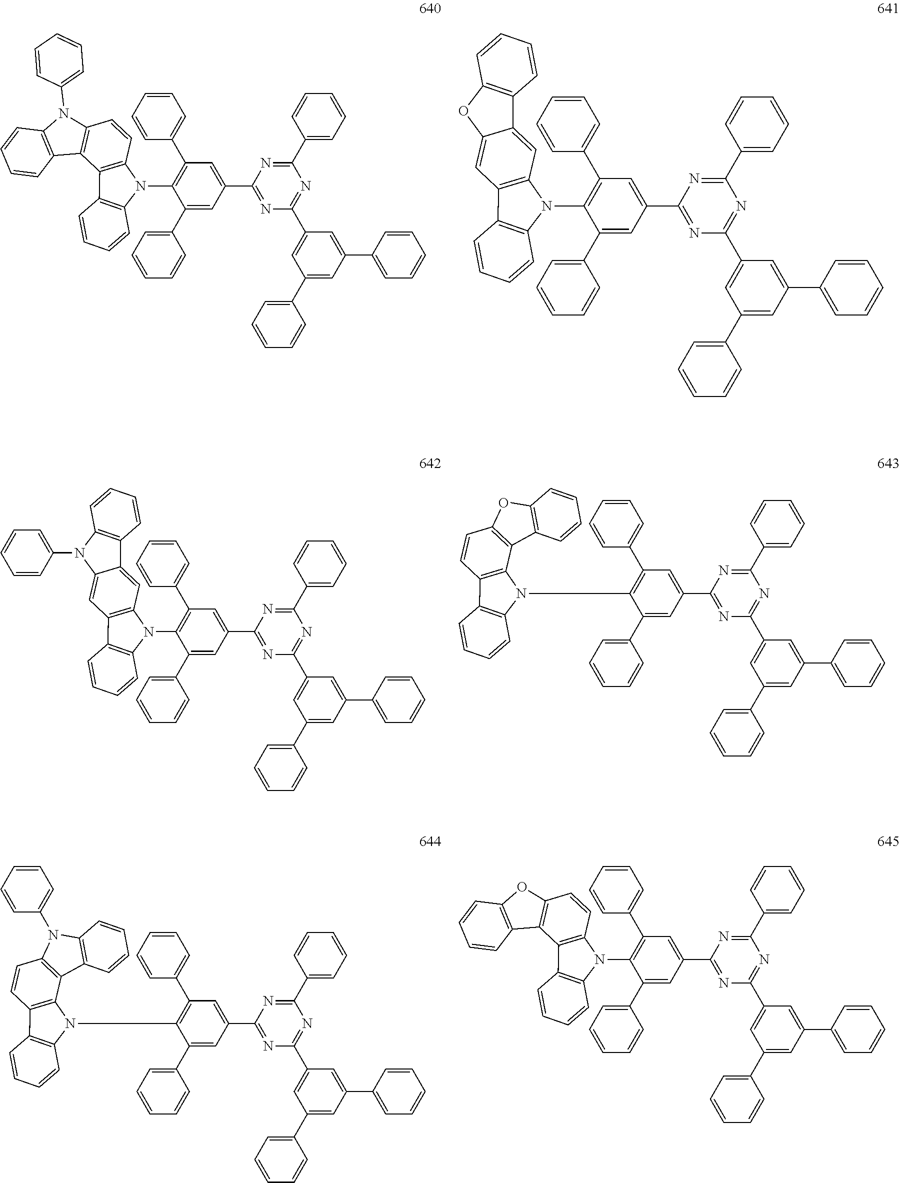

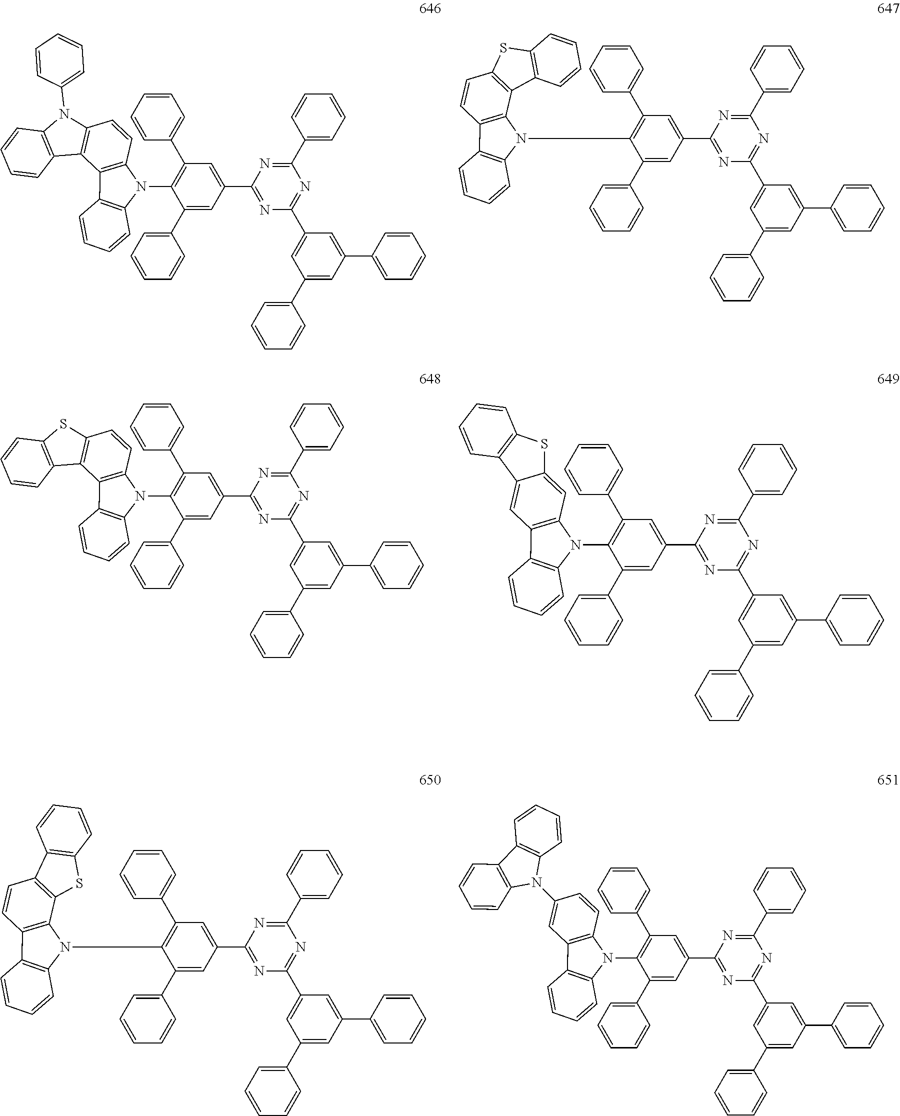

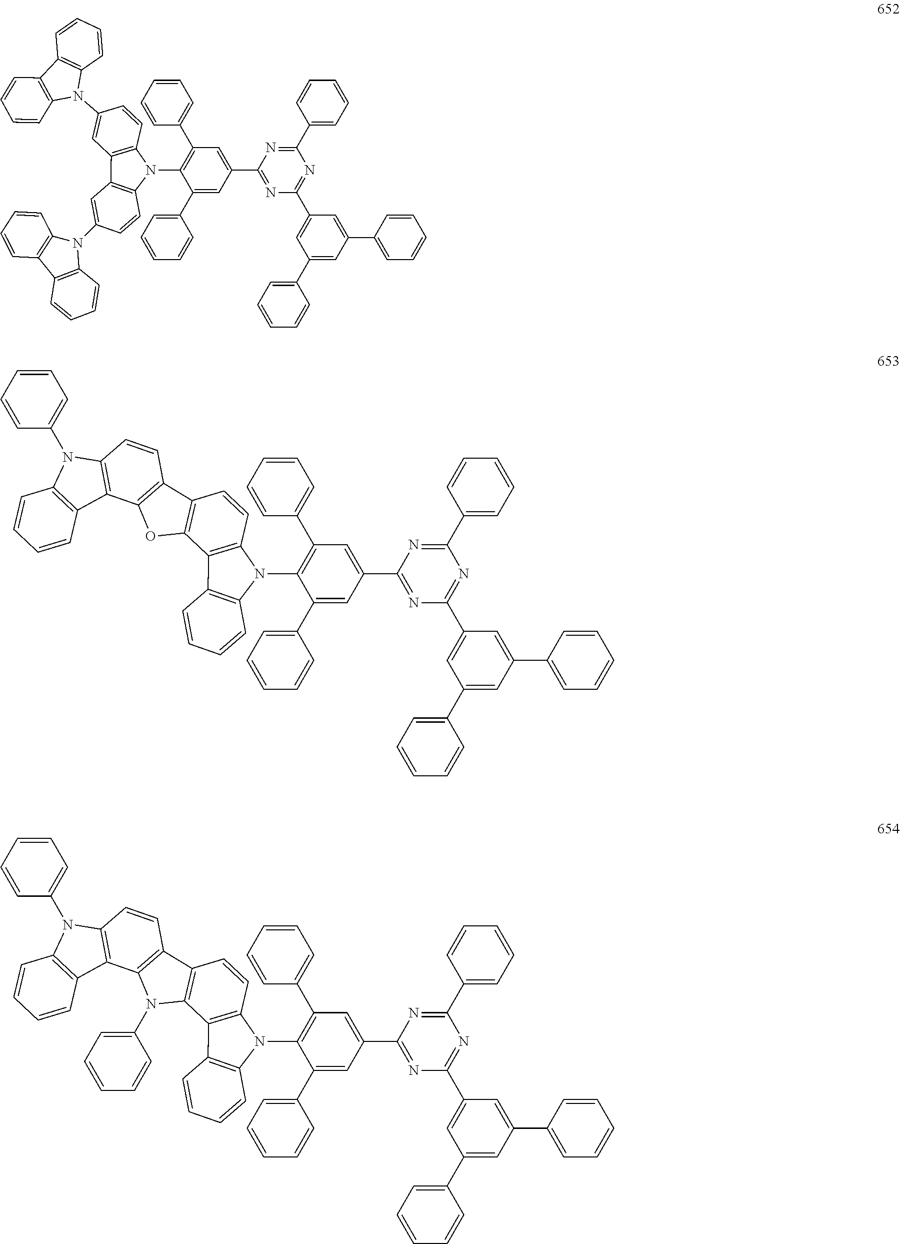 [SMILES: c1ccc(-c2cc(-c3ccccc3)cc(-c3nc(-c4ccccc4)nc(-c4cc(-c5ccccc5)c(-n5c6ccc(-n7c8ccccc8c8ccccc87)cc6c6cc(-n7c8ccccc8c8ccccc87)ccc65)c(-c5ccccc5)c4)n3)c2)cc1 ; c1ccc(-c2cc(-c3ccccc3)cc(-c3nc(-c4ccccc4)nc(-c4cc(-c5ccccc5)c(-n5c6ccccc6c6c7oc8c(ccc9c8c8ccccc8n9-c8ccccc8)c7ccc65)c(-c5ccccc5)c4)n3)c2)cc1 ; c1ccc(-c2cc(-c3ccccc3)cc(-c3nc(-c4ccccc4)nc(-c4cc(-c5ccccc5)c(-n5c6ccccc6c6c5ccc5c7ccc8c(c9ccccc9n8-c8ccccc8)c7n(-c7ccccc7)c56)c(-c5ccccc5)c4)n3)c2)cc1]

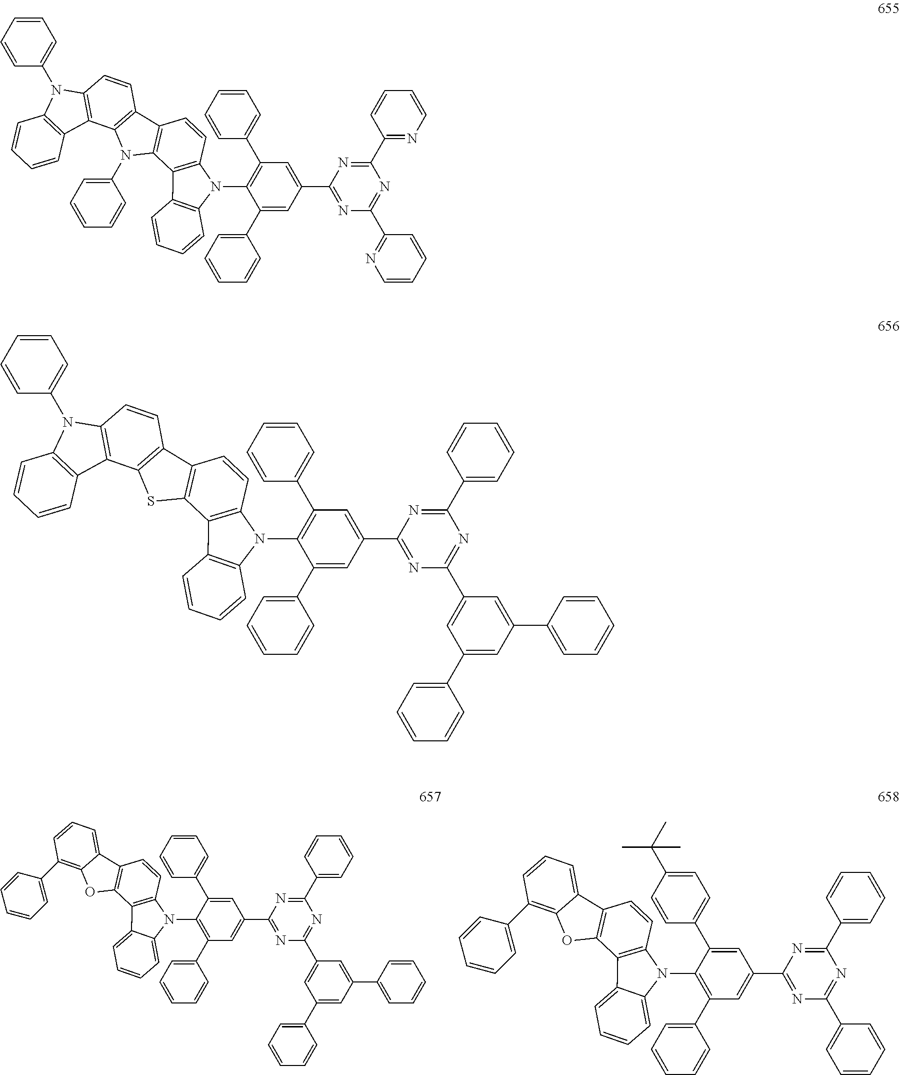

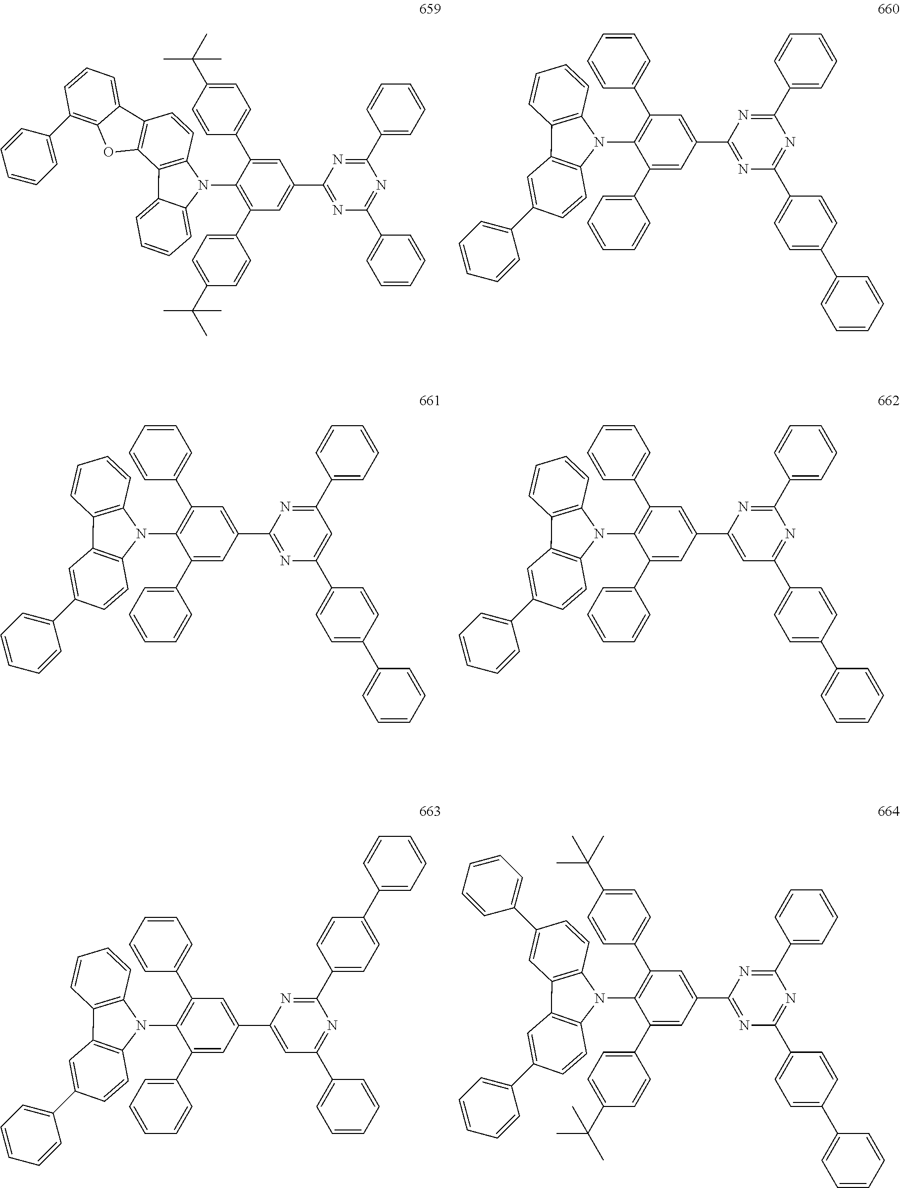

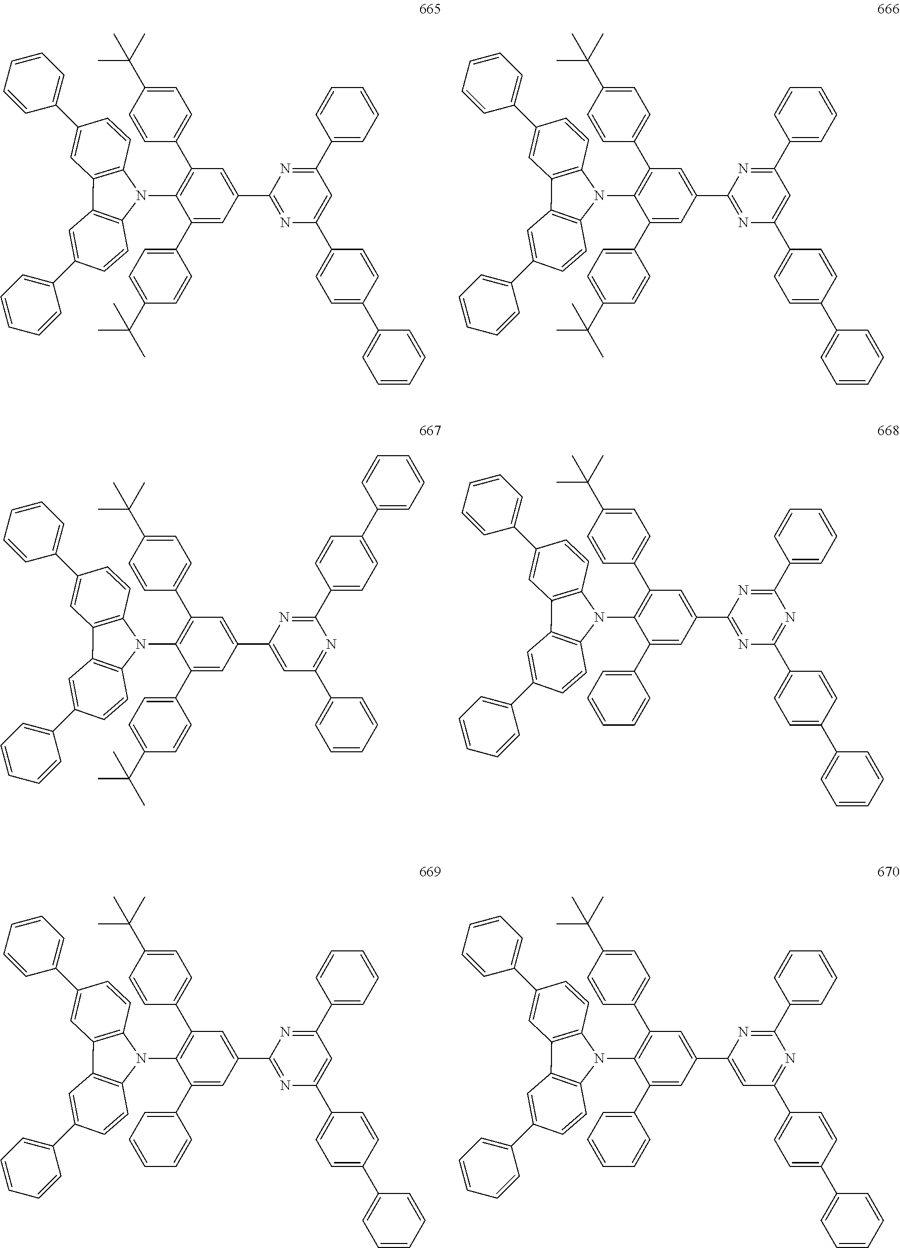

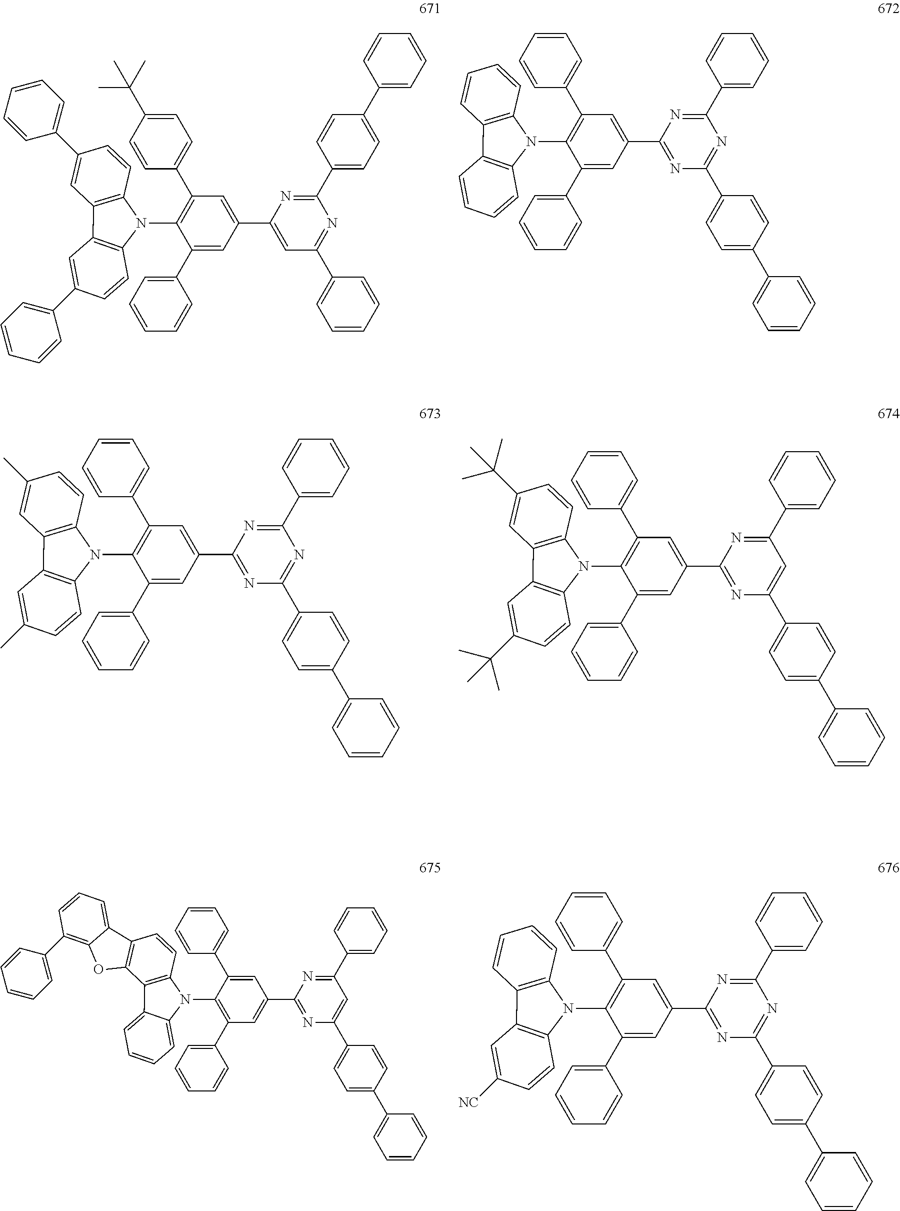

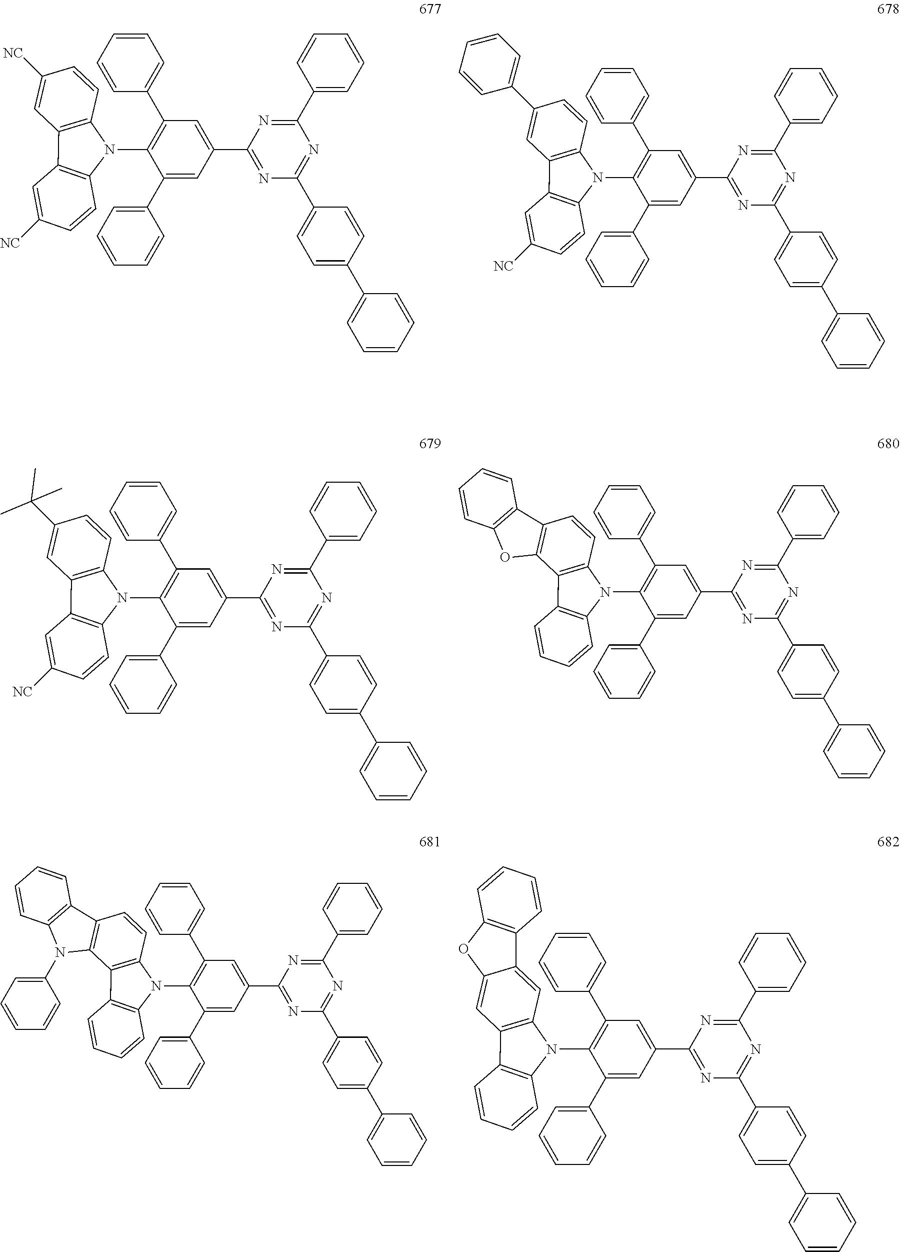

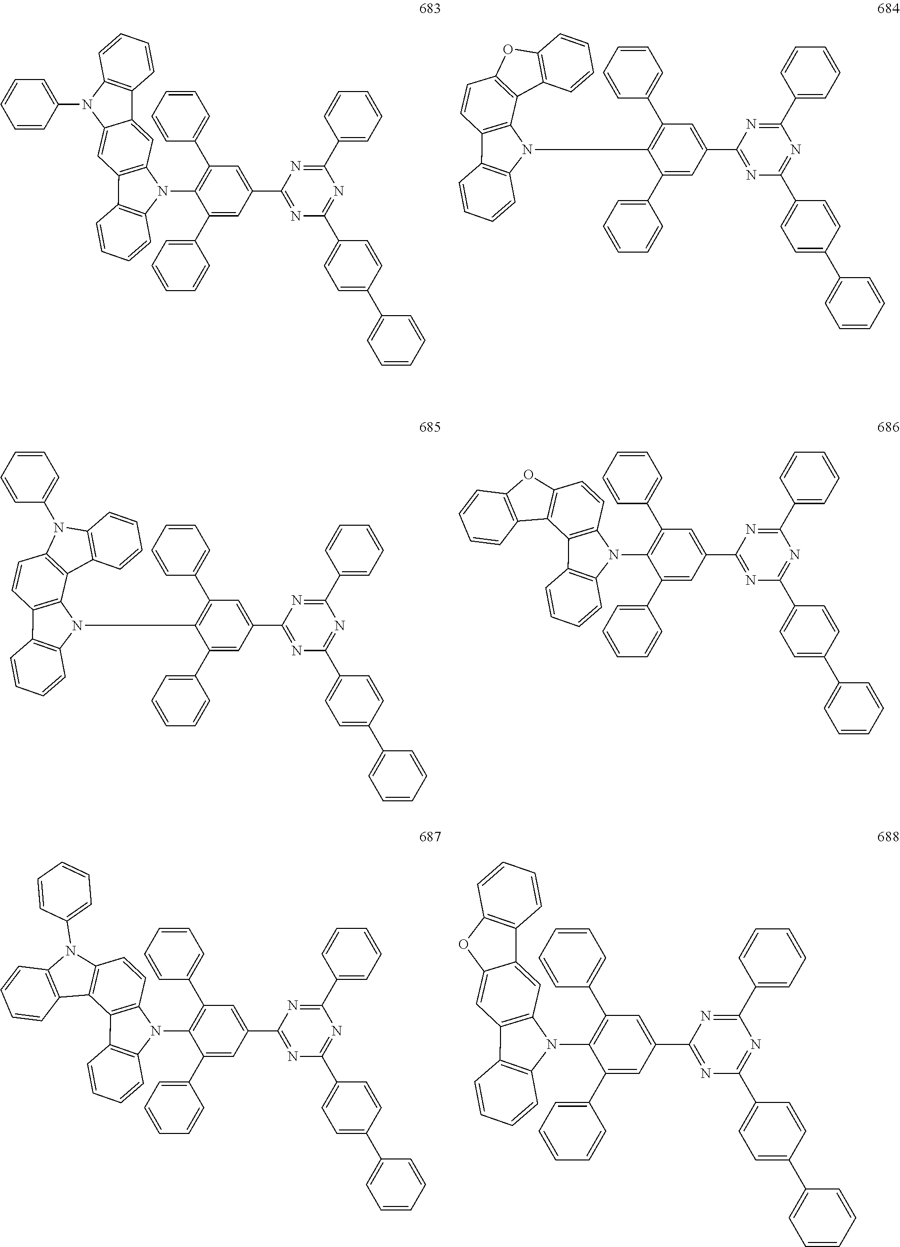

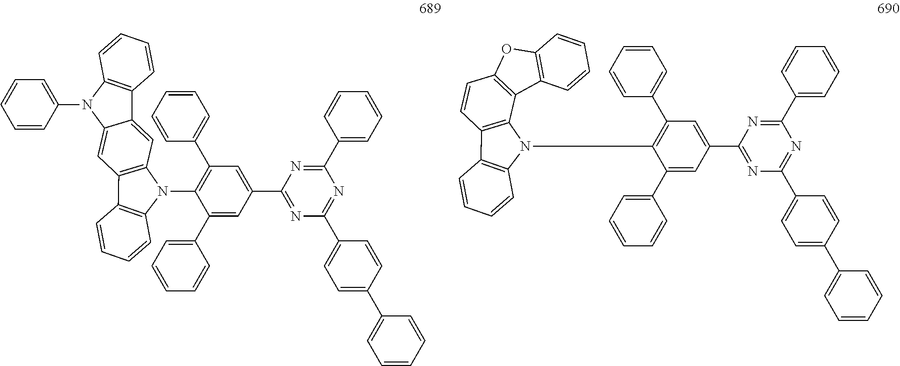 [SMILES: c1ccc(-c2ccc(-c3nc(-c4ccccc4)nc(-c4cc(-c5ccccc5)c(-n5c6ccccc6c6cc7c(cc65)c5ccccc5n7-c5ccccc5)c(-c5ccccc5)c4)n3)cc2)cc1 ; c1ccc(-c2ccc(-c3nc(-c4ccccc4)nc(-c4cc(-c5ccccc5)c(-n5c6ccccc6c6ccc7oc8ccccc8c7c65)c(-c5ccccc5)c4)n3)cc2)cc1]

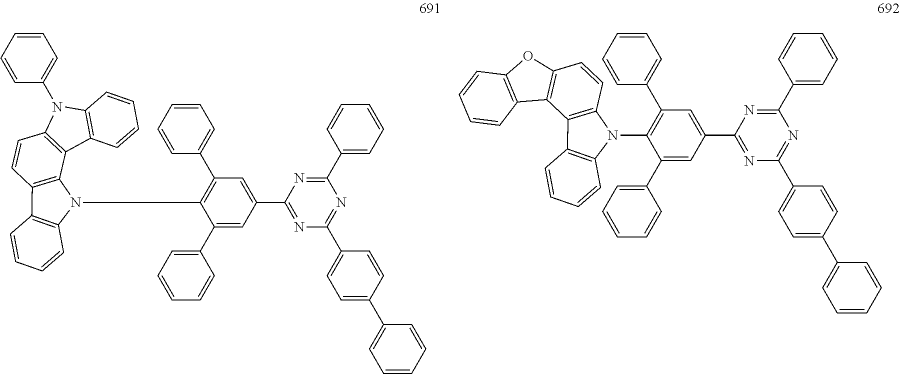 [SMILES: c1ccc(-c2ccc(-c3nc(-c4ccccc4)nc(-c4cc(-c5ccccc5)c(-n5c6ccccc6c6ccc7c(c8ccccc8n7-c7ccccc7)c65)c(-c5ccccc5)c4)n3)cc2)cc1 ; c1ccc(-c2ccc(-c3nc(-c4ccccc4)nc(-c4cc(-c5ccccc5)c(-n5c6ccccc6c6c7c(ccc65)oc5ccccc57)c(-c5ccccc5)c4)n3)cc2)cc1]

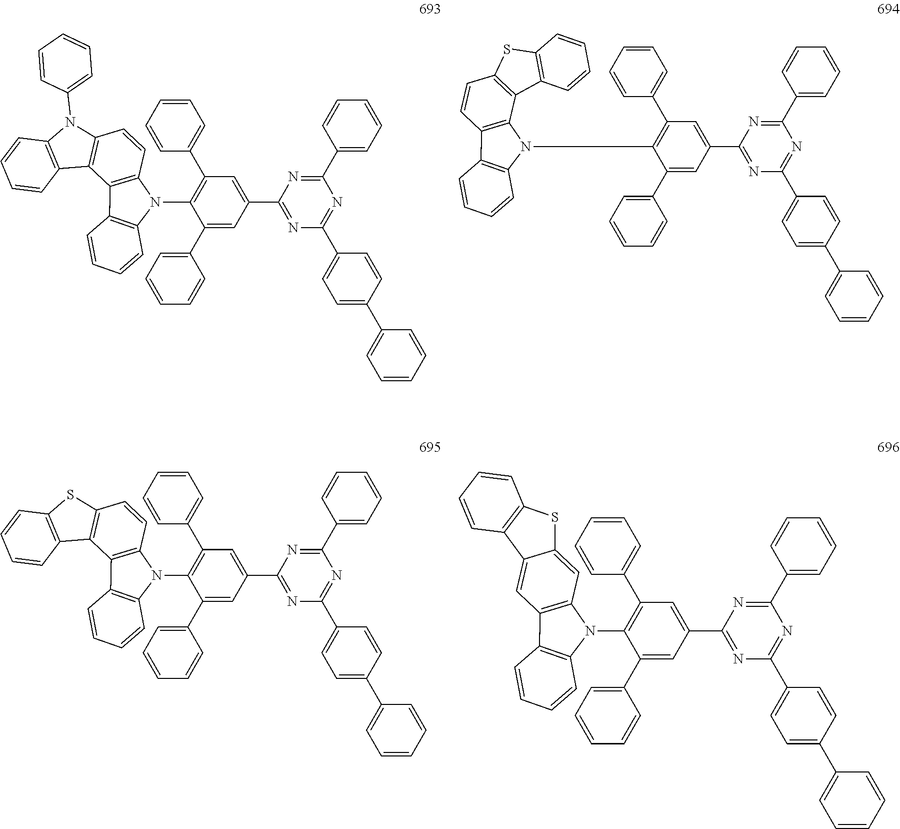 [SMILES: c1ccc(-c2ccc(-c3nc(-c4ccccc4)nc(-c4cc(-c5ccccc5)c(-n5c6ccccc6c6c7c8ccccc8n(-c8ccccc8)c7ccc65)c(-c5ccccc5)c4)n3)cc2)cc1 ; c1ccc(-c2ccc(-c3nc(-c4ccccc4)nc(-c4cc(-c5ccccc5)c(-n5c6ccccc6c6ccc7sc8ccccc8c7c65)c(-c5ccccc5)c4)n3)cc2)cc1 ; c1ccc(-c2ccc(-c3nc(-c4ccccc4)nc(-c4cc(-c5ccccc5)c(-n5c6ccccc6c6c7c(ccc65)sc5ccccc57)c(-c5ccccc5)c4)n3)cc2)cc1 ; c1ccc(-c2ccc(-c3nc(-c4ccccc4)nc(-c4cc(-c5ccccc5)c(-n5c6ccccc6c6cc7c(cc65)sc5ccccc57)c(-c5ccccc5)c4)n3)cc2)cc1]

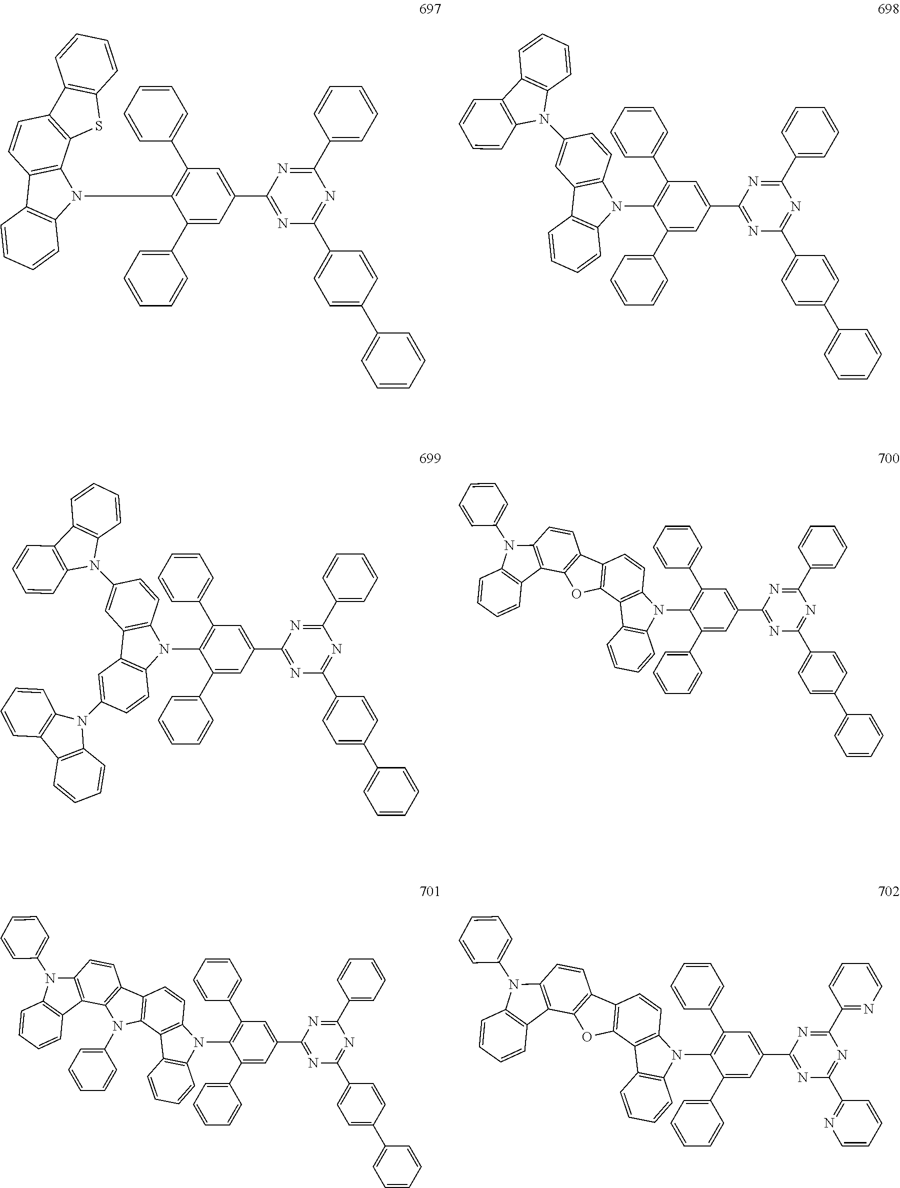

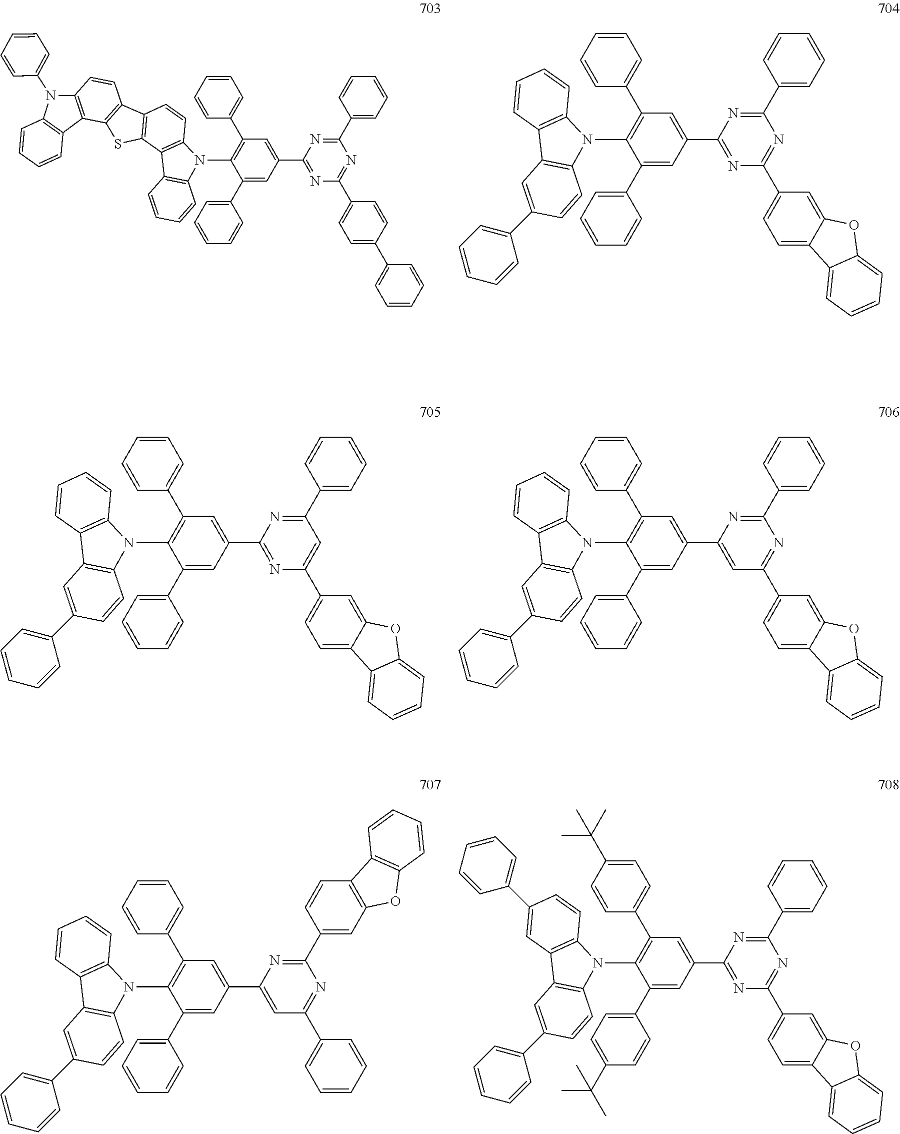

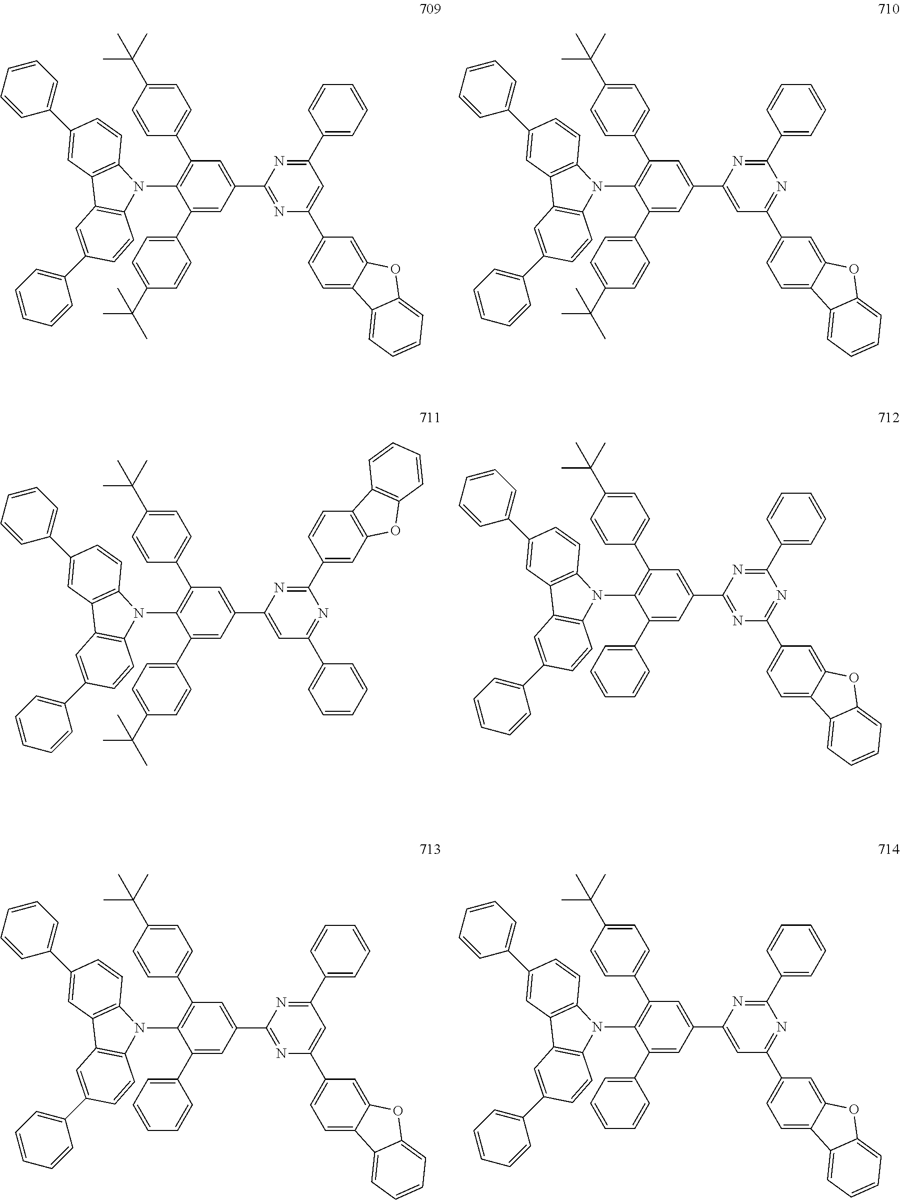

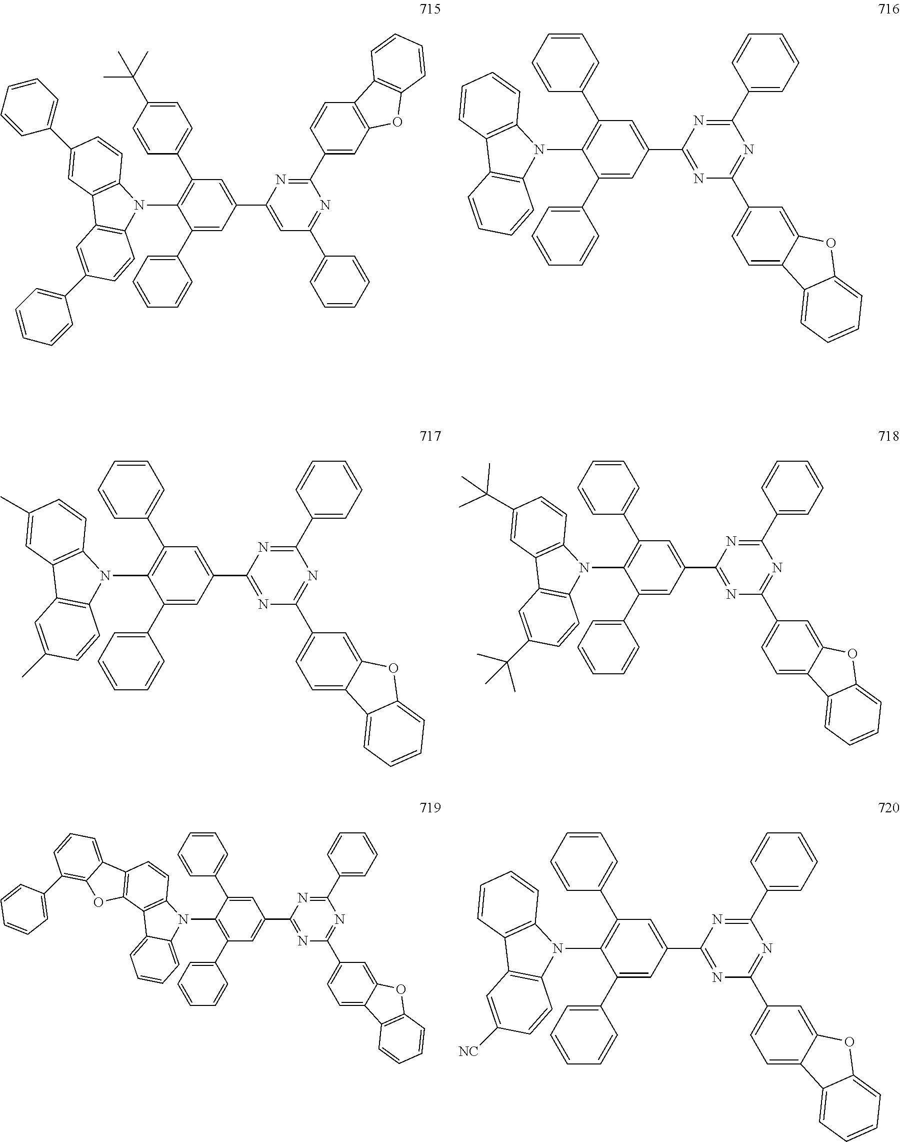

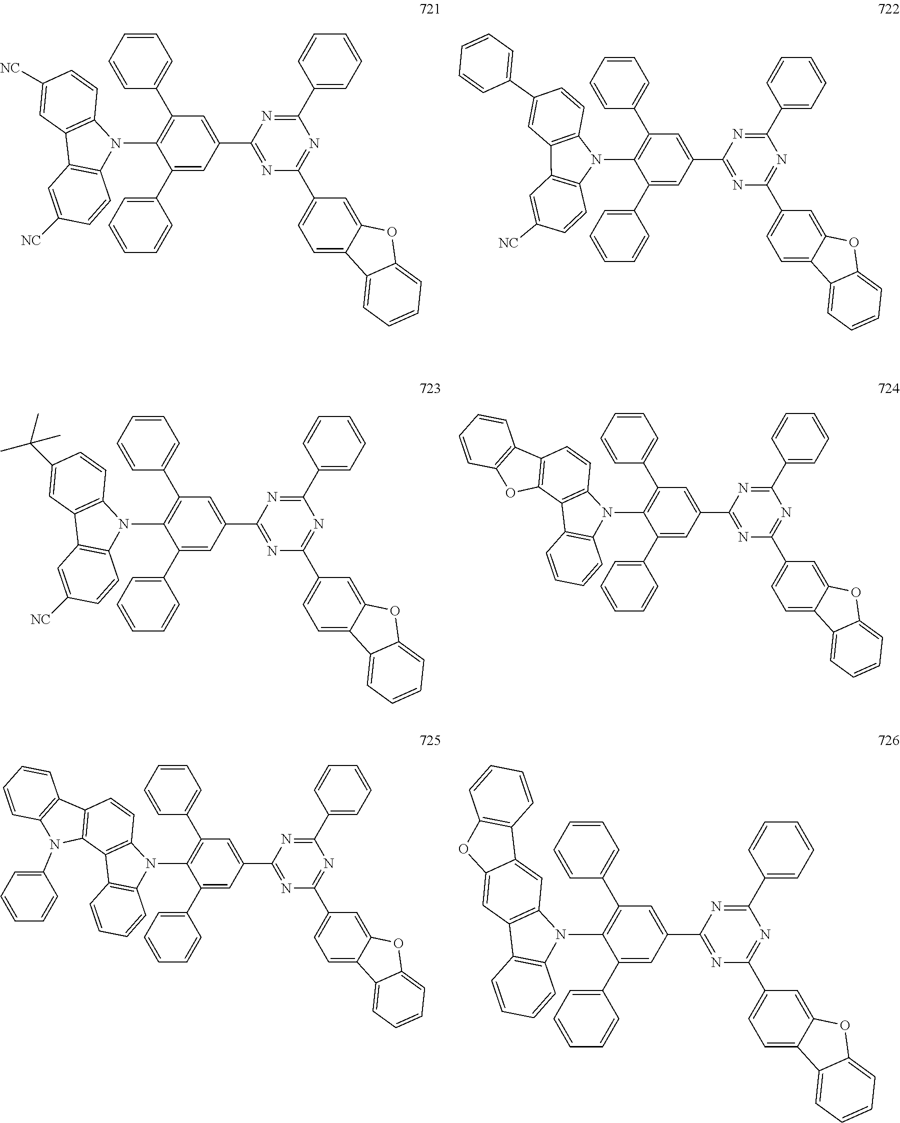

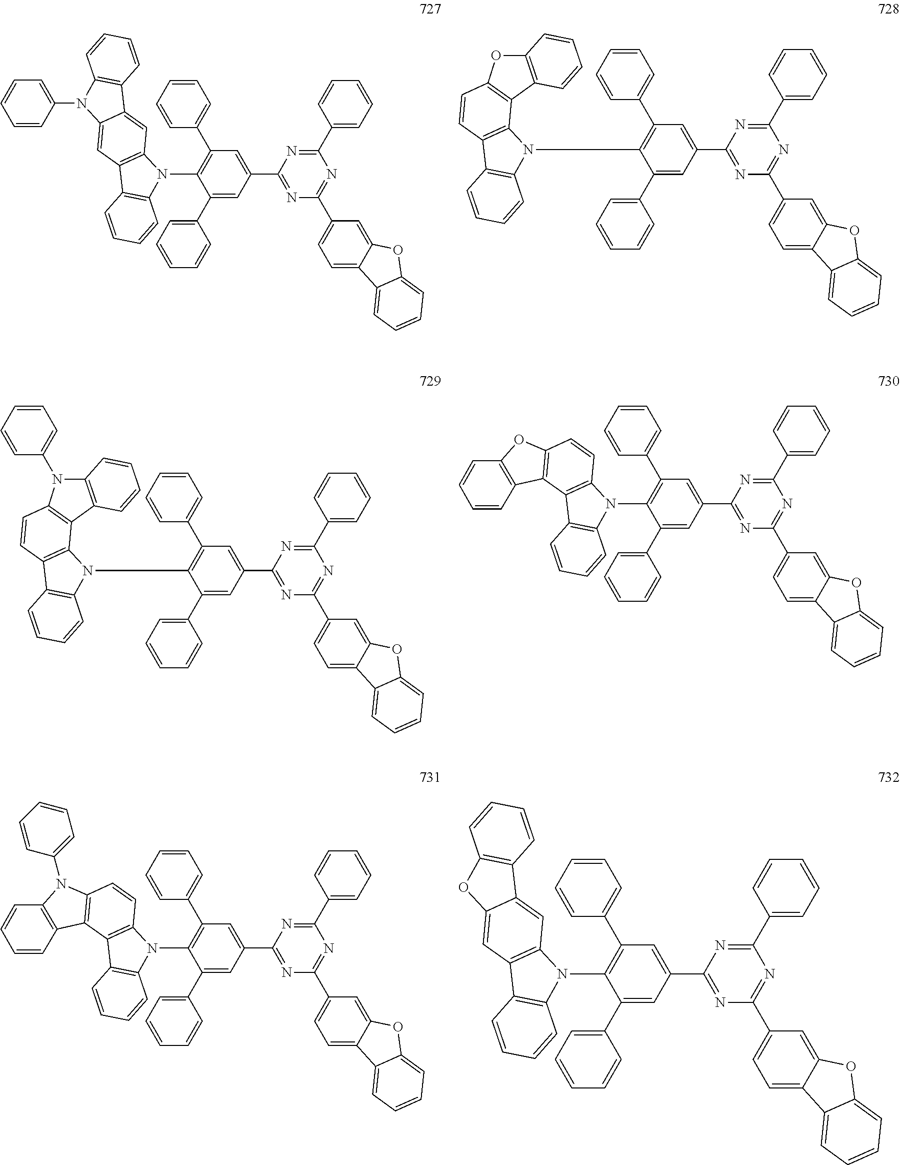

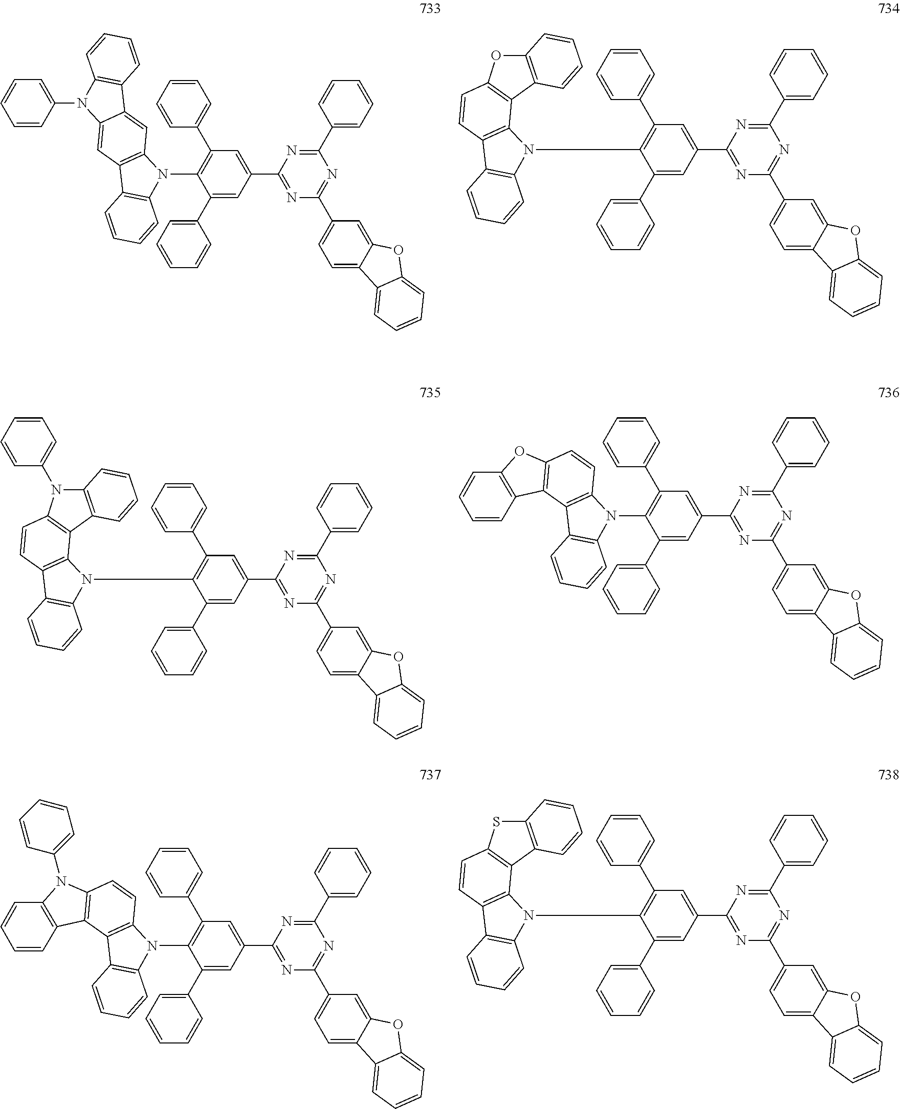

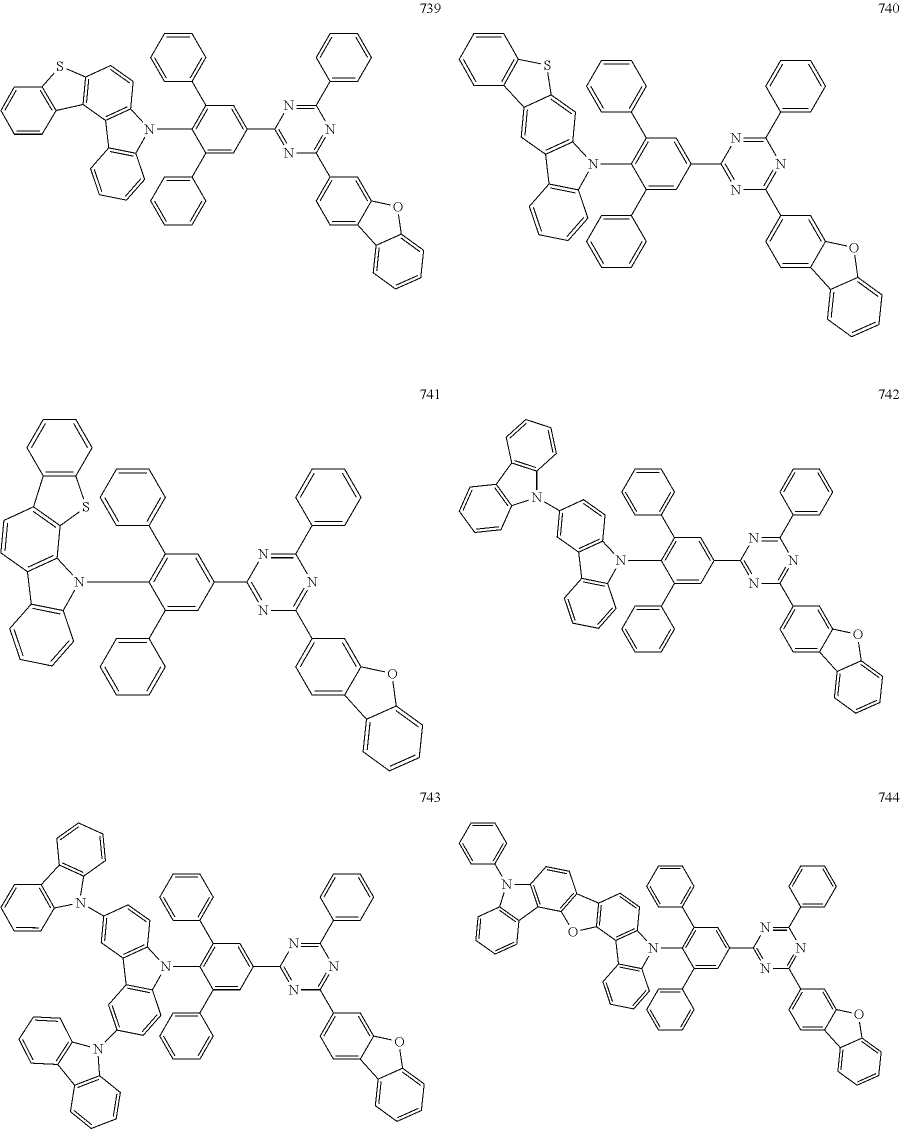

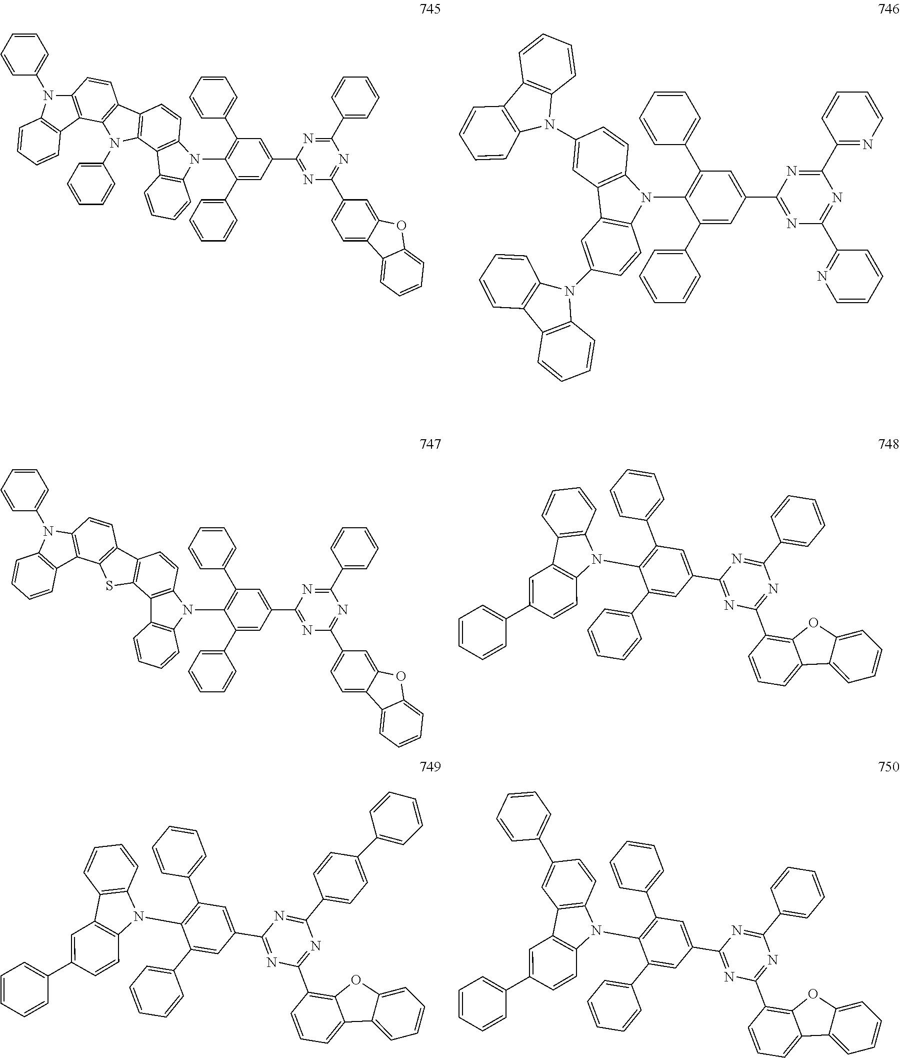

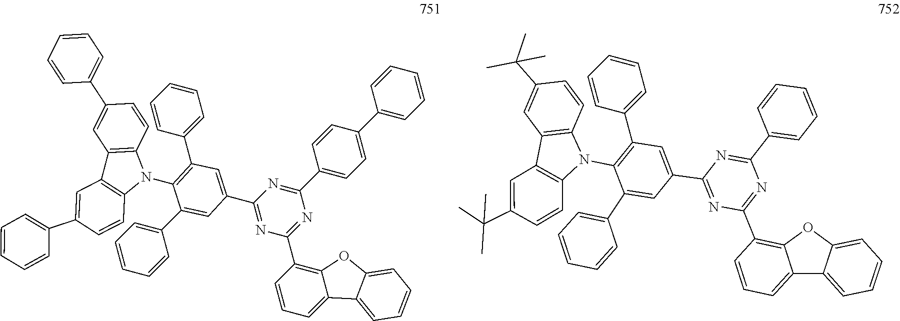 [SMILES: c1ccc(-c2ccc(-c3nc(-c4cc(-c5ccccc5)c(-n5c6ccc(-c7ccccc7)cc6c6cc(-c7ccccc7)ccc65)c(-c5ccccc5)c4)nc(-c4cccc5c4oc4ccccc45)n3)cc2)cc1 ; CC(C)(C)c1ccc2c(c1)c1cc(C(C)(C)C)ccc1n2-c1c(-c2ccccc2)cc(-c2nc(-c3ccccc3)nc(-c3cccc4c3oc3ccccc34)n2)cc1-c1ccccc1]

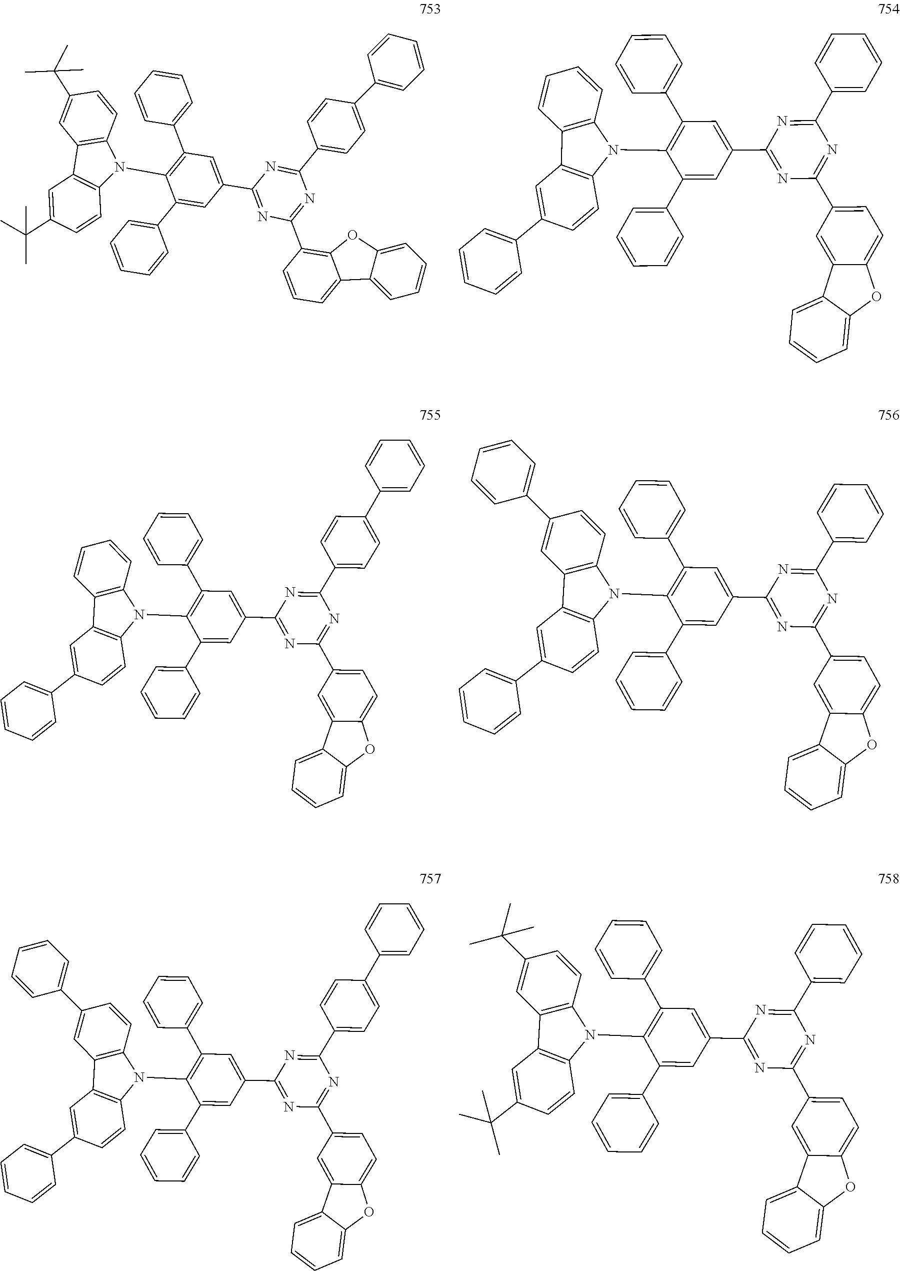

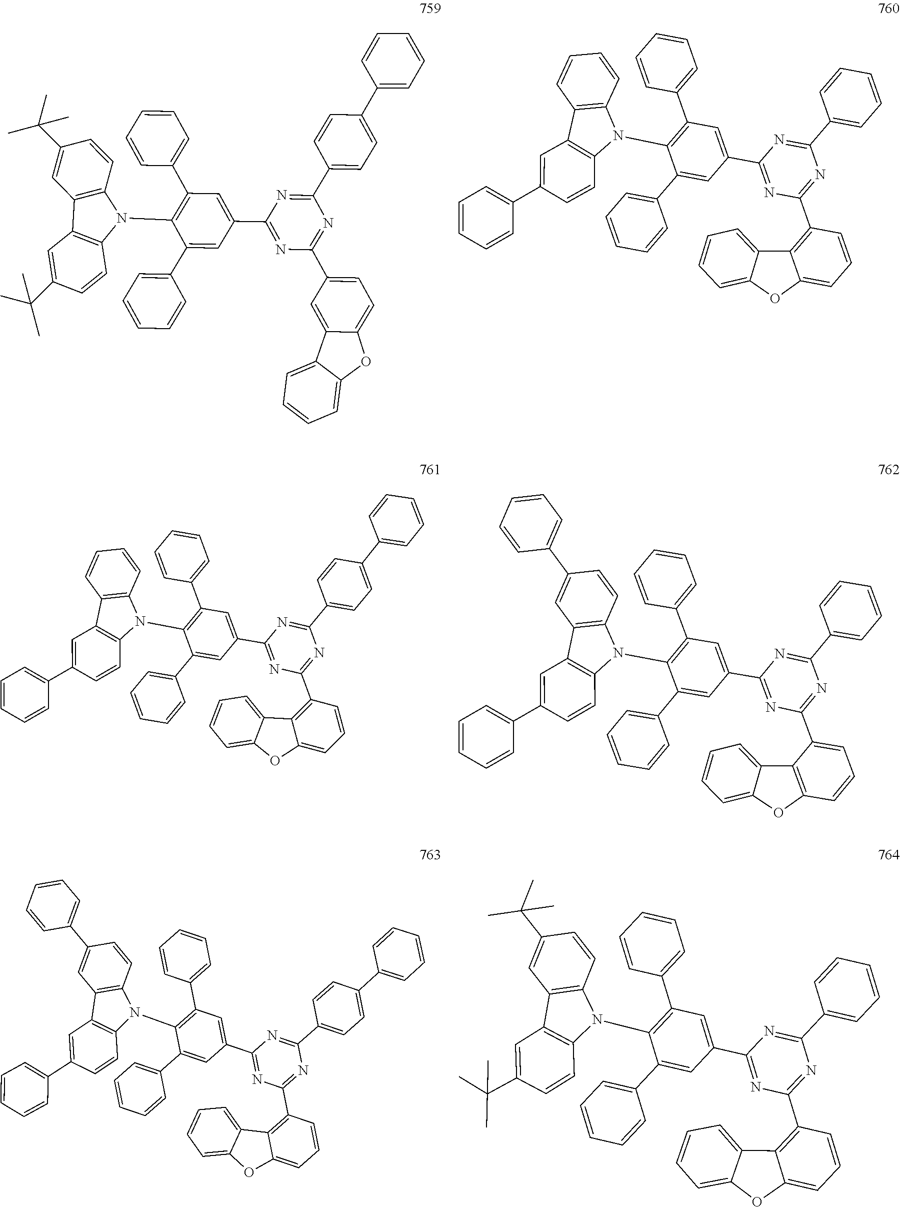

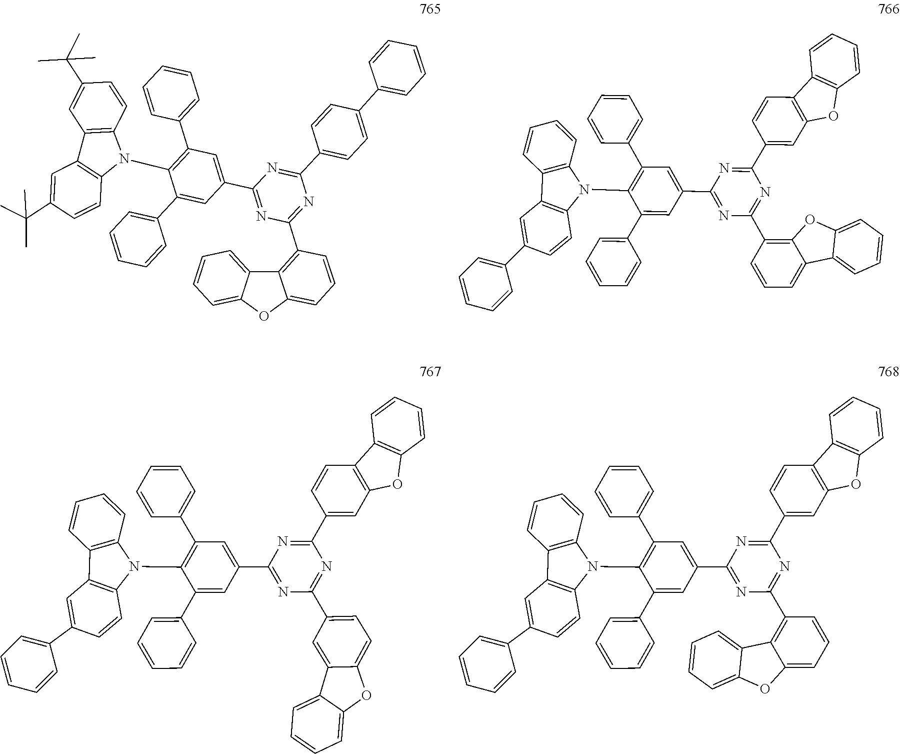 [SMILES: CC(C)(C)c1ccc2c(c1)c1cc(C(C)(C)C)ccc1n2-c1c(-c2ccccc2)cc(-c2nc(-c3ccc(-c4ccccc4)cc3)nc(-c3cccc4oc5ccccc5c34)n2)cc1-c1ccccc1 ; c1ccc(-c2ccc3c(c2)c2ccccc2n3-c2c(-c3ccccc3)cc(-c3nc(-c4ccc5c(c4)oc4ccccc45)nc(-c4cccc5c4oc4ccccc45)n3)cc2-c2ccccc2)cc1 ; c1ccc(-c2ccc3c(c2)c2ccccc2n3-c2c(-c3ccccc3)cc(-c3nc(-c4ccc5c(c4)oc4ccccc45)nc(-c4ccc5oc6ccccc6c5c4)n3)cc2-c2ccccc2)cc1 ; c1ccc(-c2ccc3c(c2)c2ccccc2n3-c2c(-c3ccccc3)cc(-c3nc(-c4ccc5c(c4)oc4ccccc45)nc(-c4cccc5oc6ccccc6c45)n3)cc2-c2ccccc2)cc1]

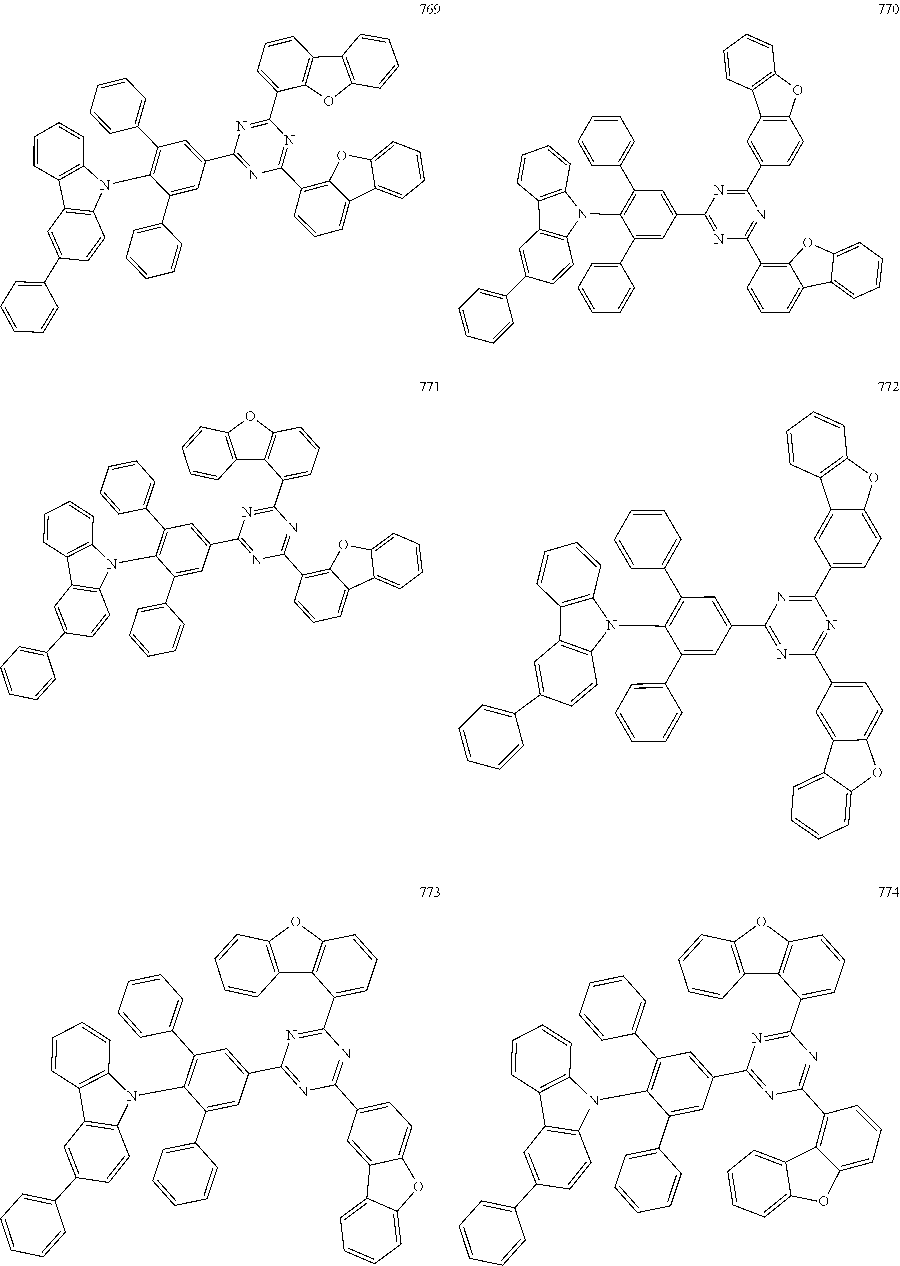

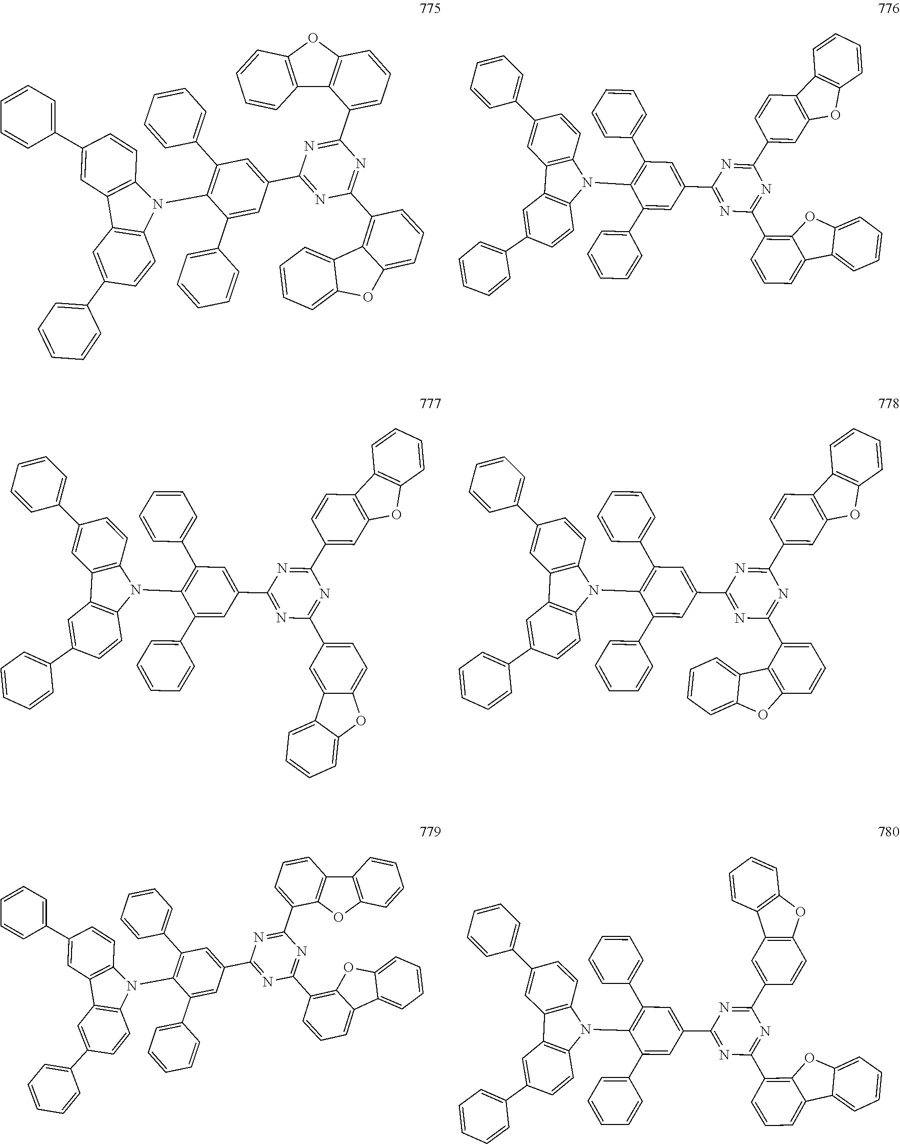

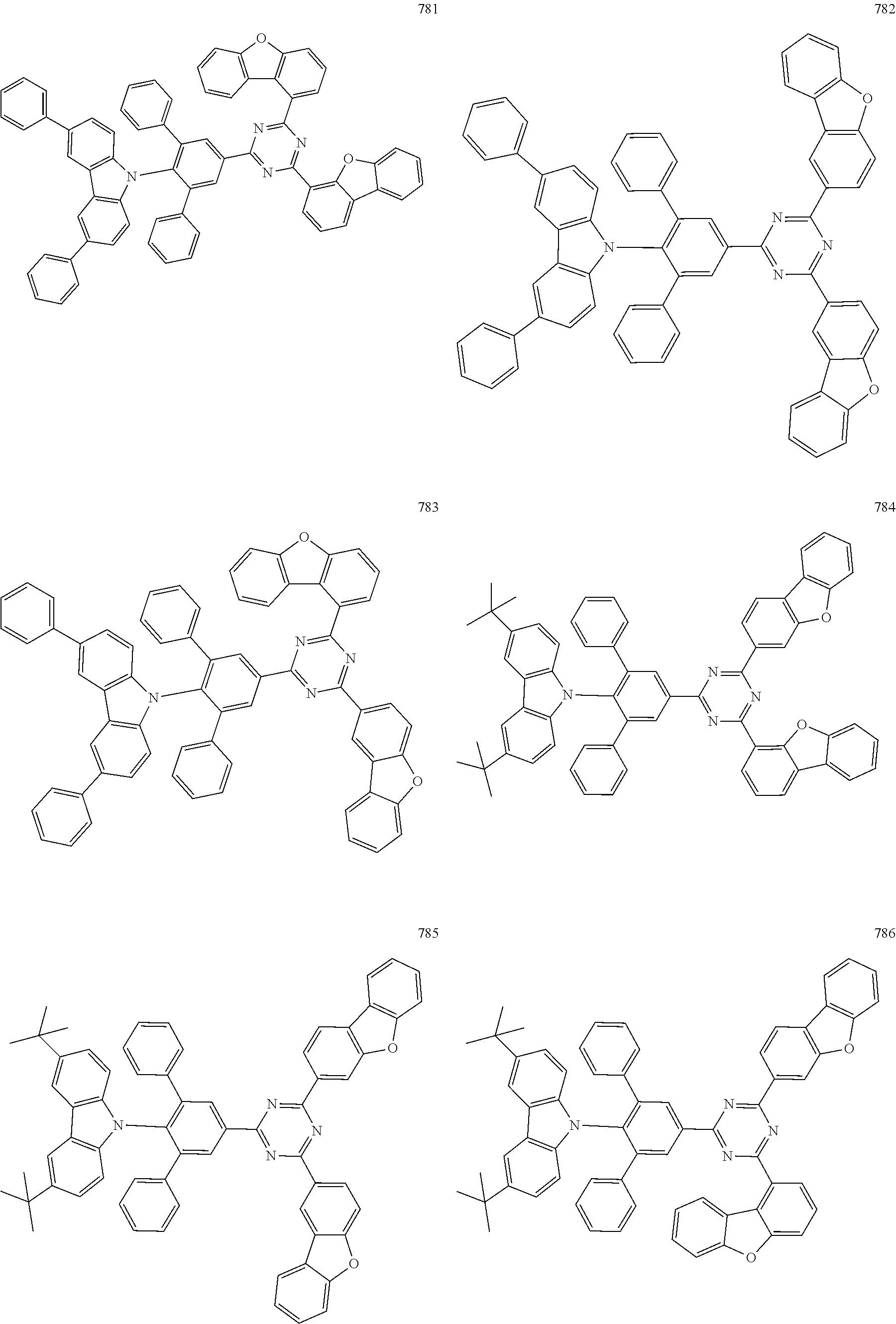

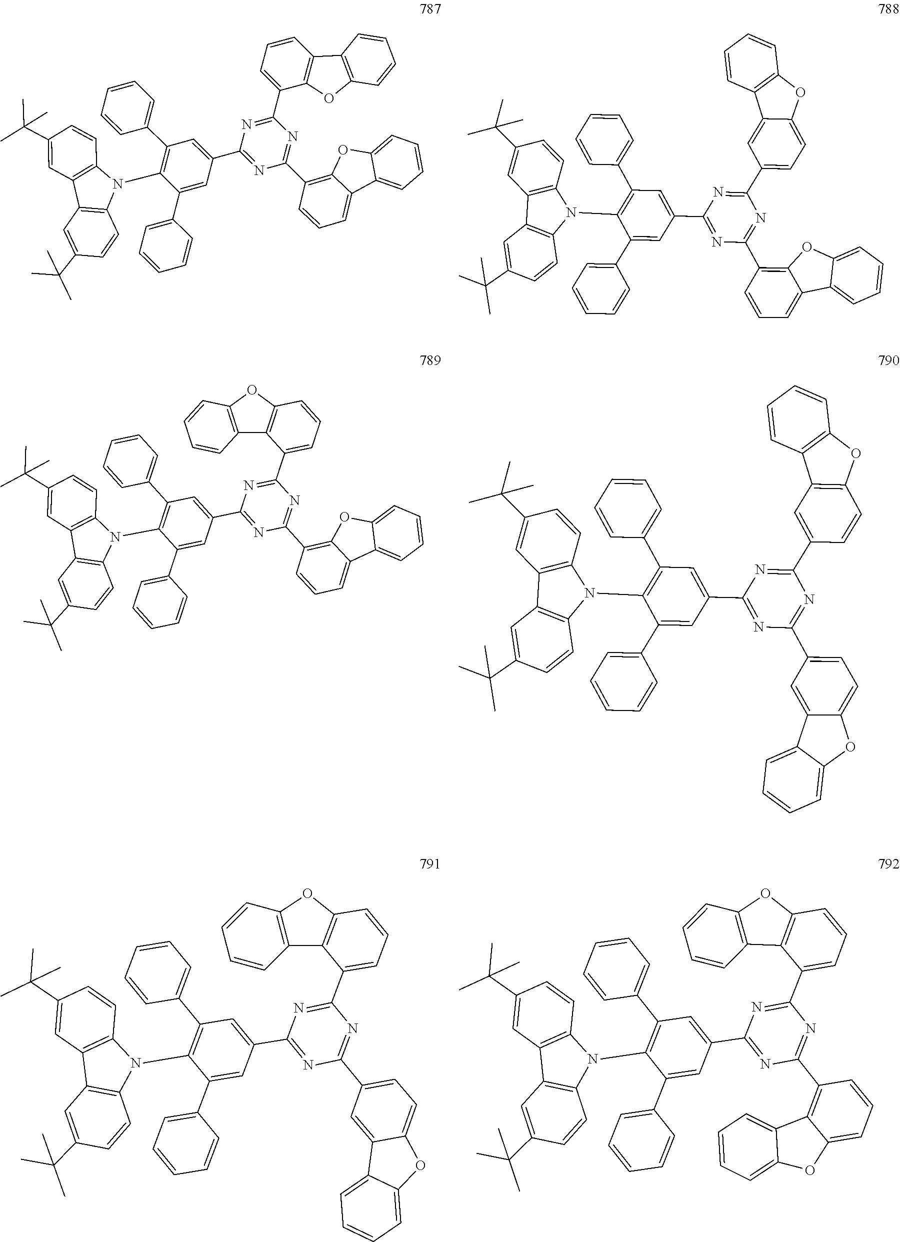

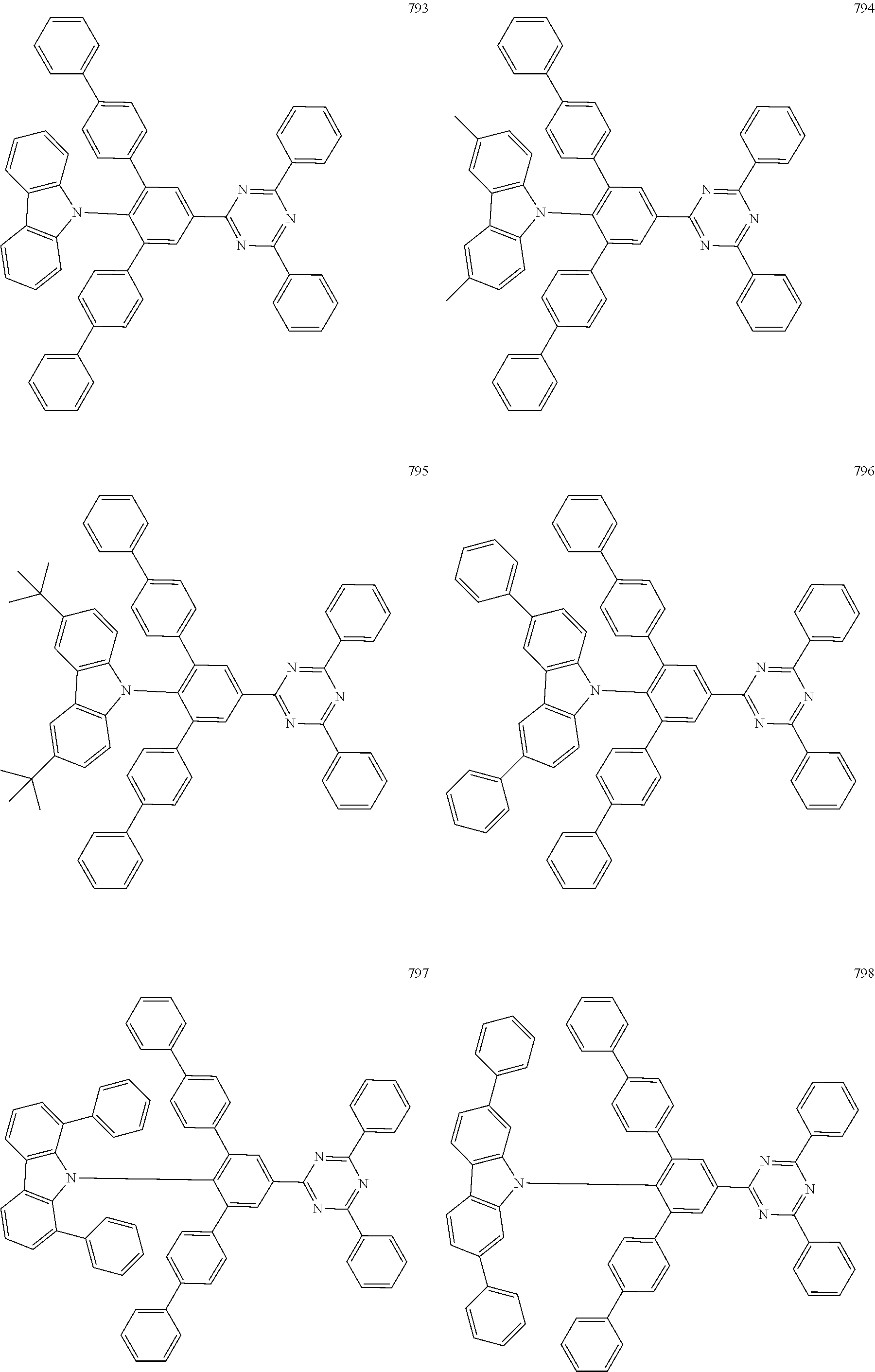

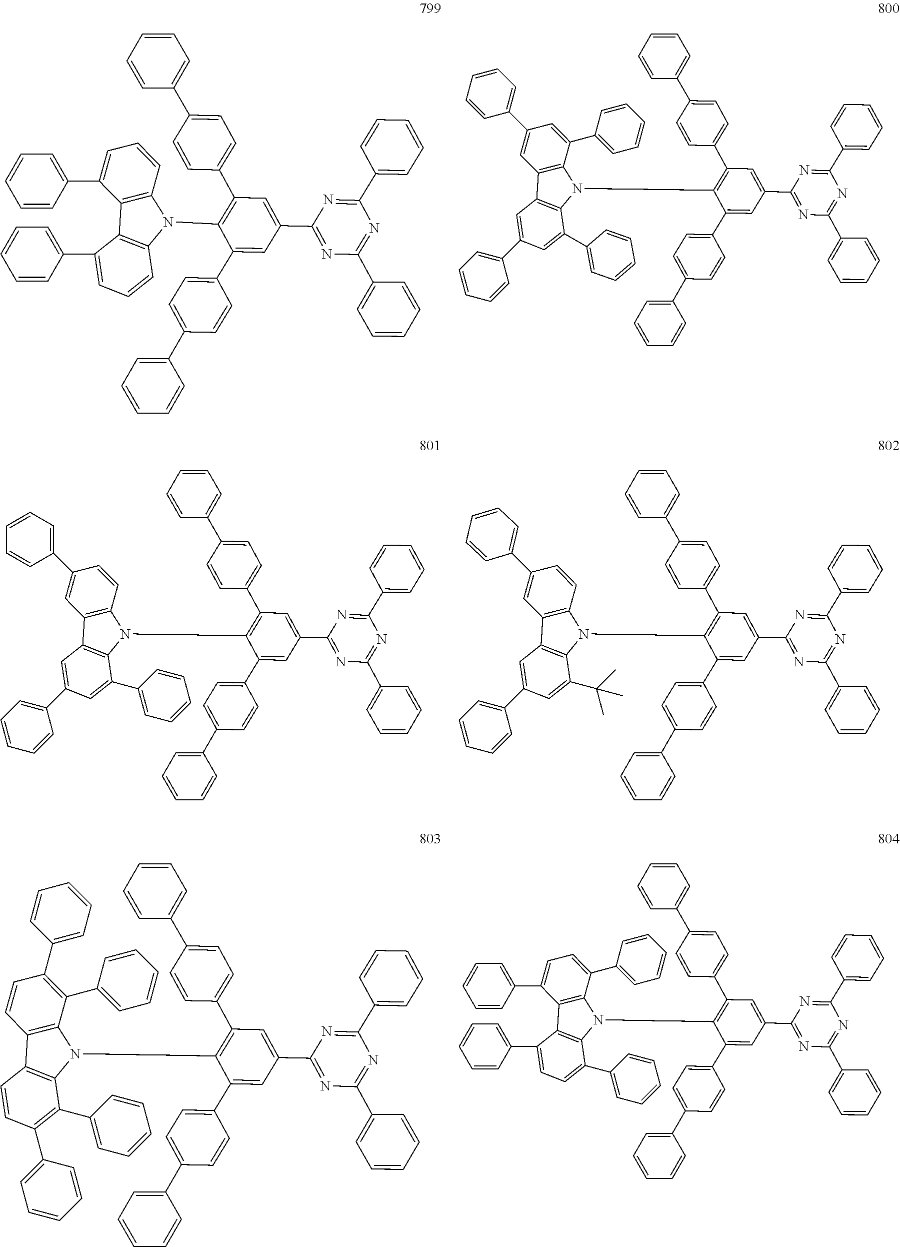

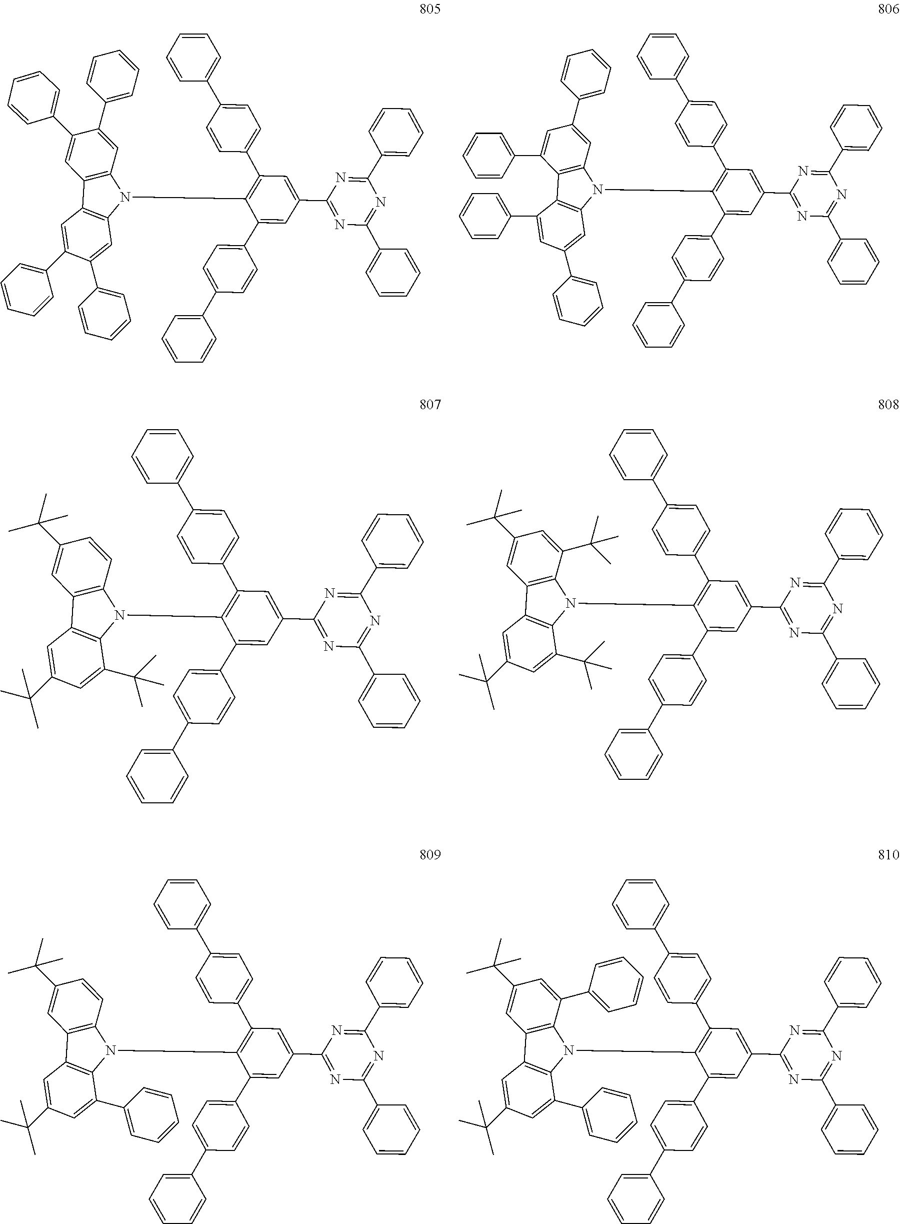

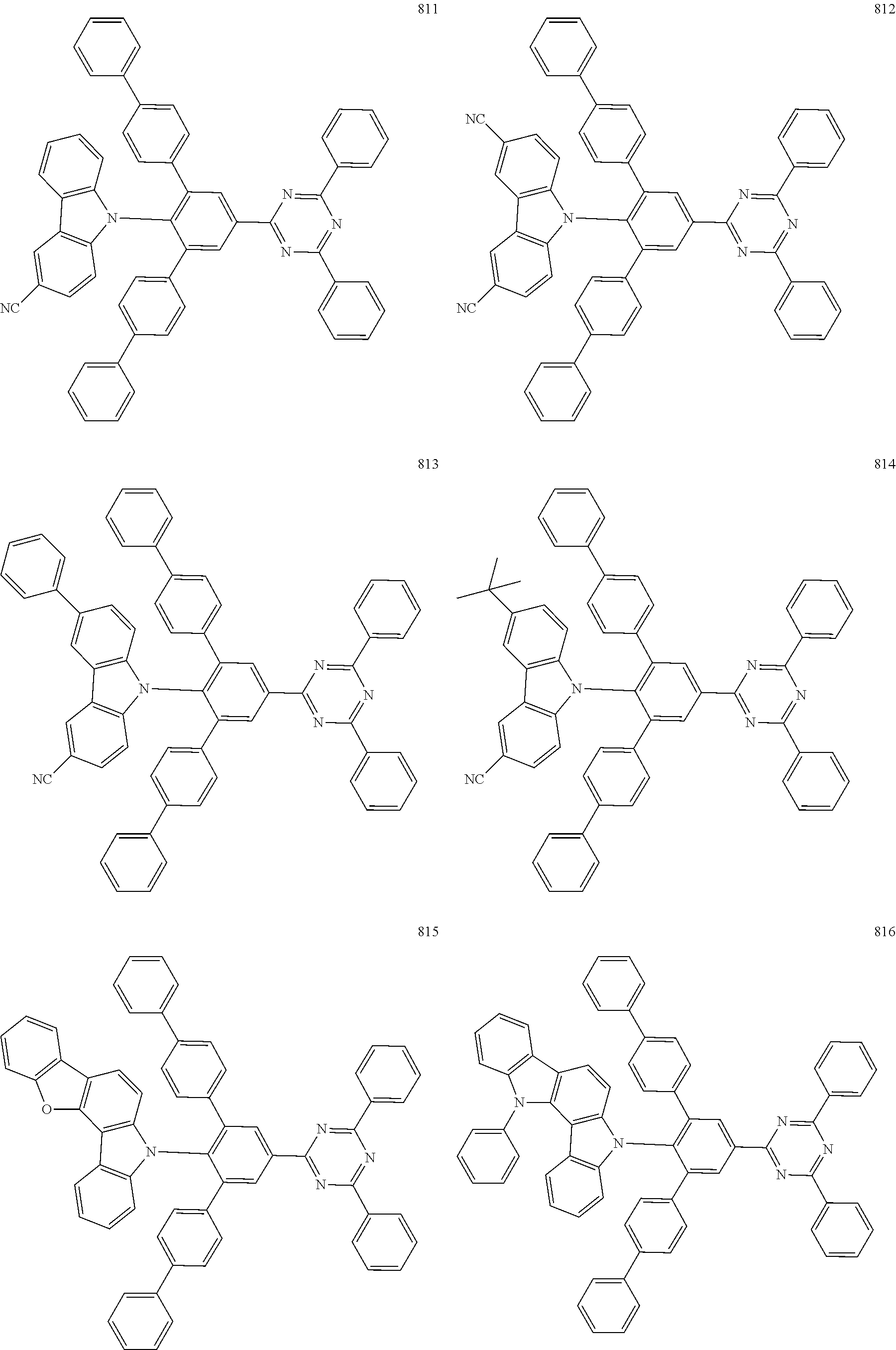

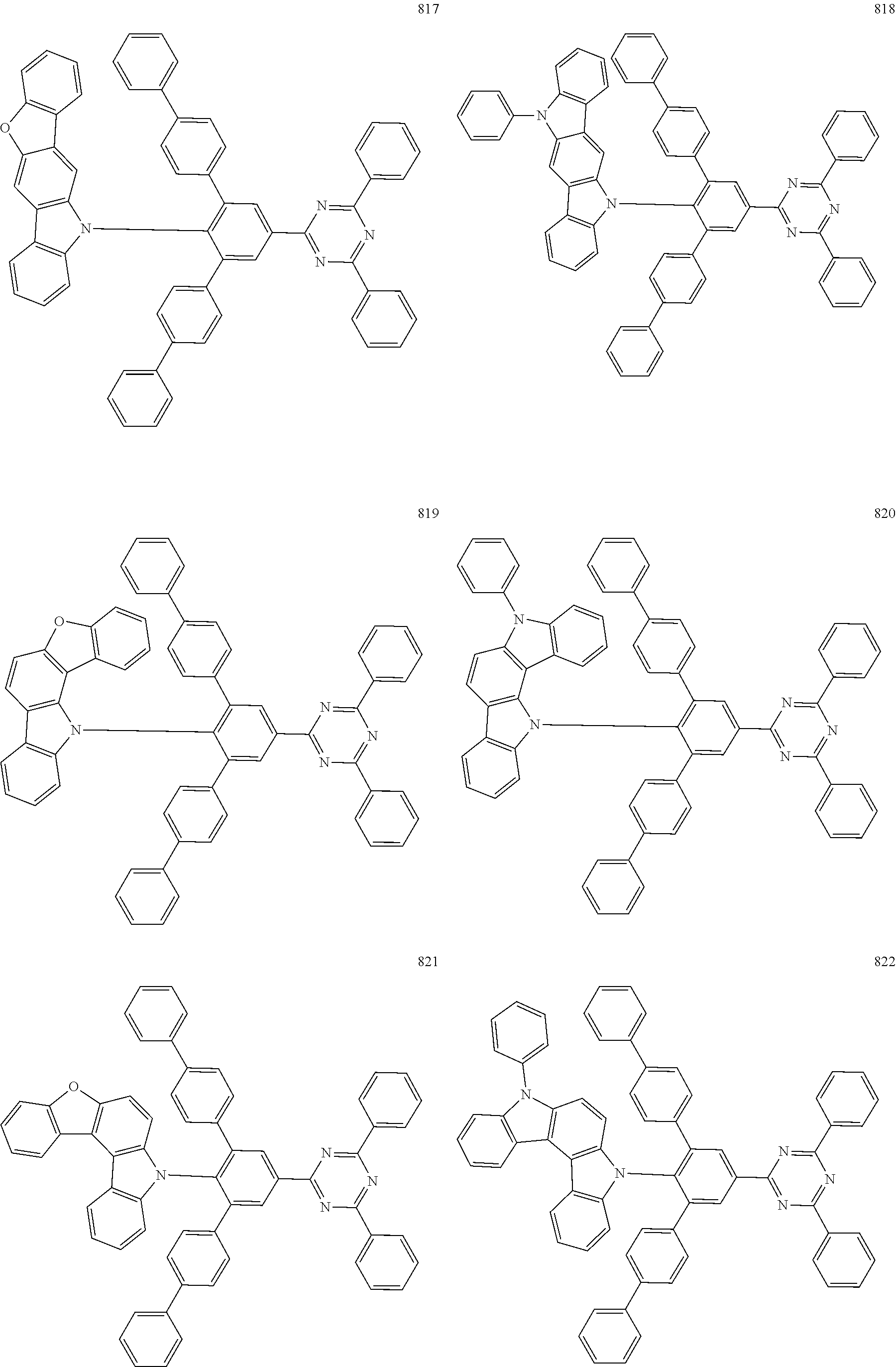

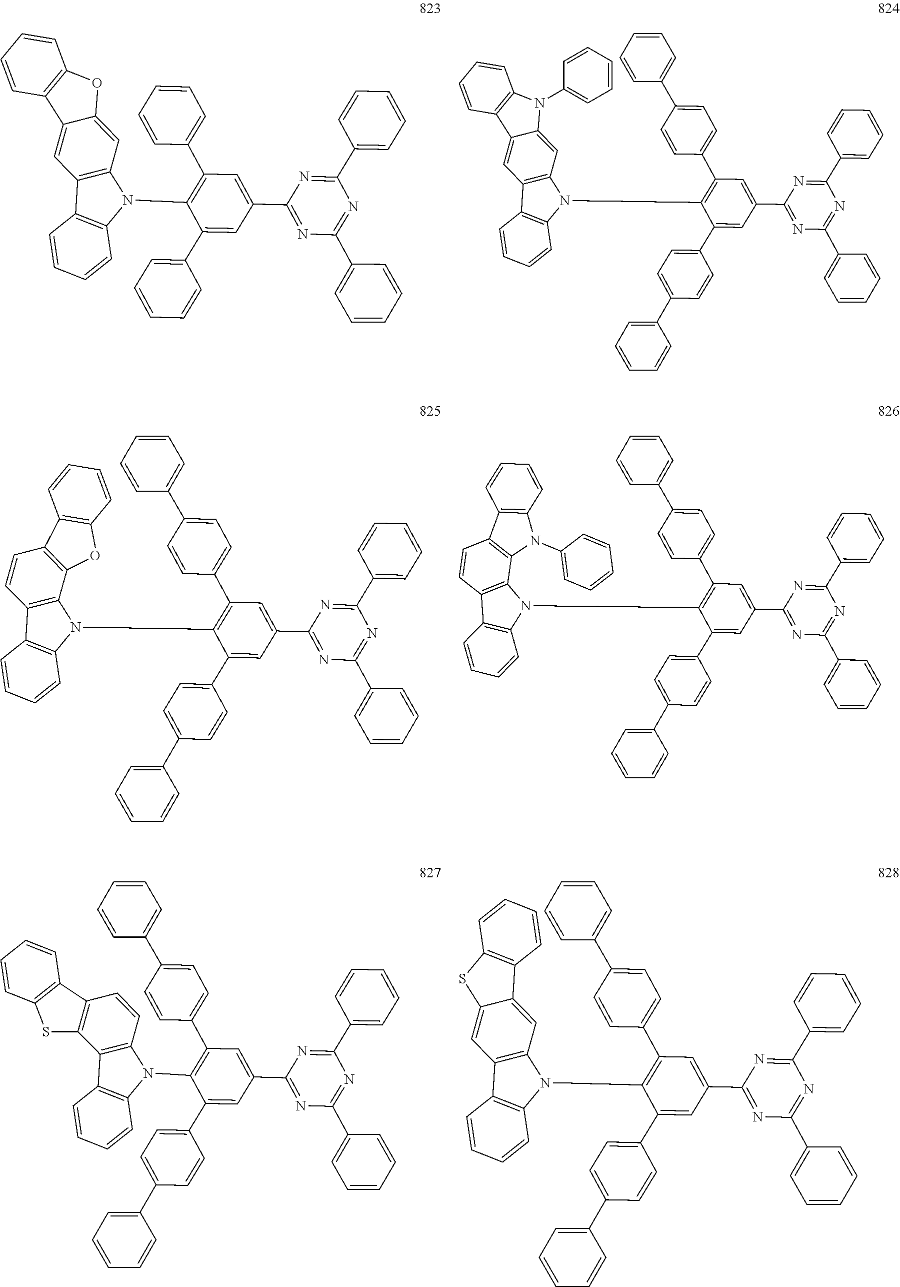

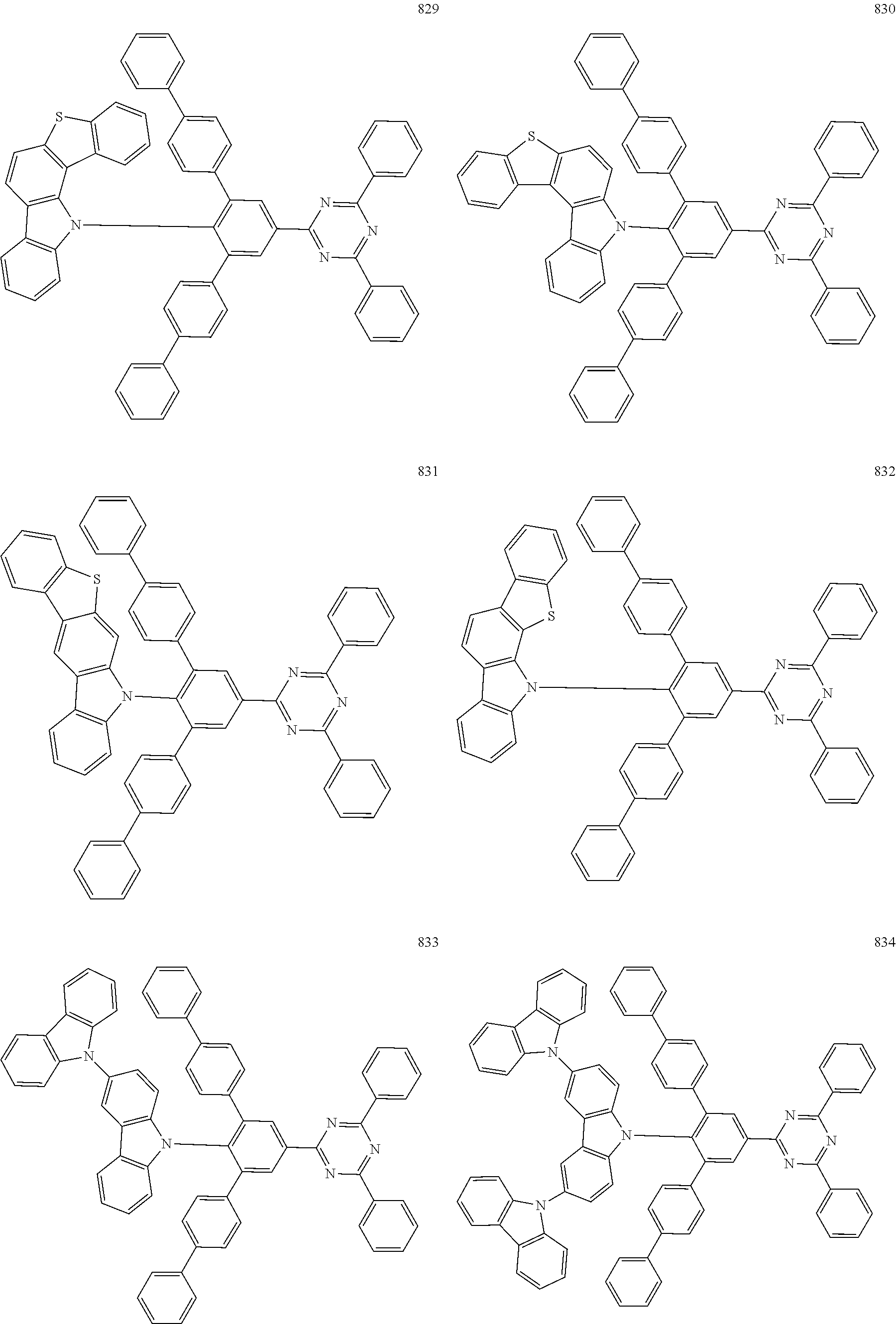

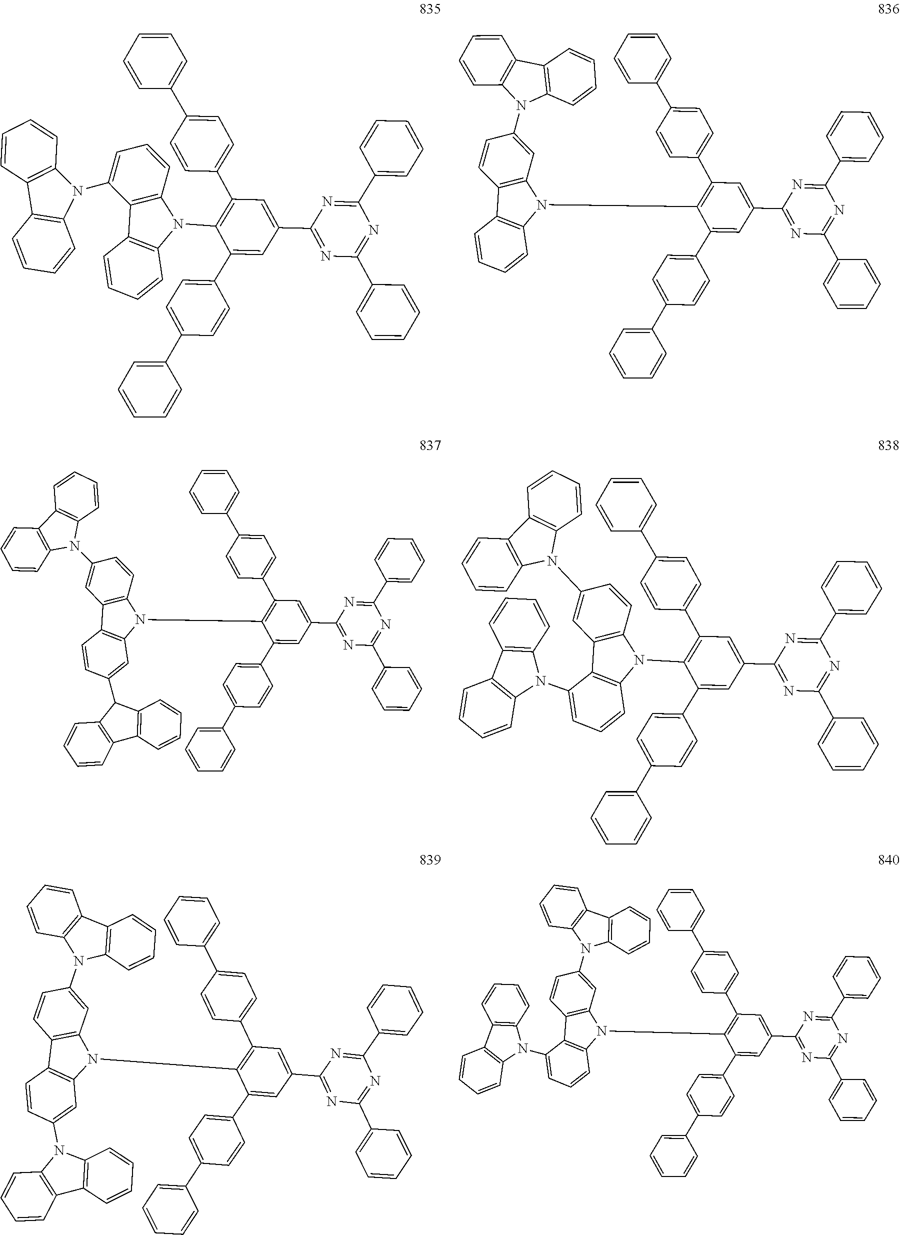

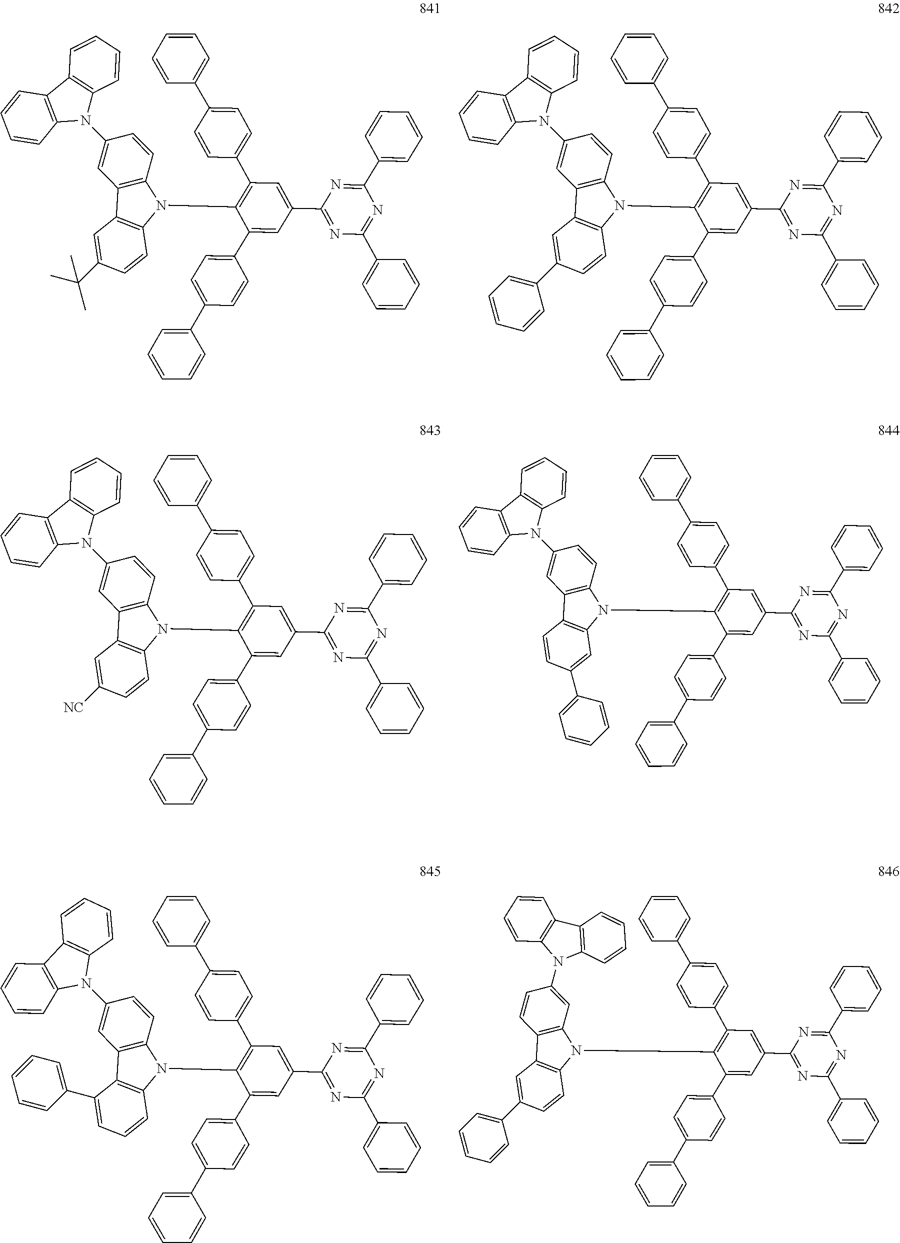

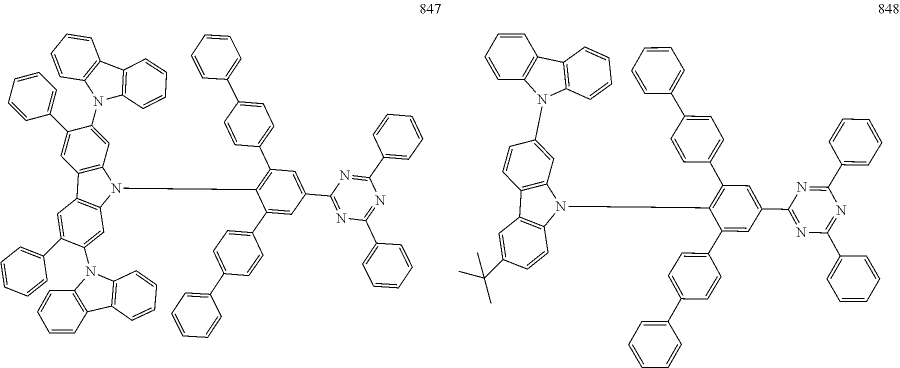 [SMILES: c1ccc(-c2ccc(-c3cc(-c4nc(-c5ccccc5)nc(-c5ccccc5)n4)cc(-c4ccc(-c5ccccc5)cc4)c3-n3c4cc(-n5c6ccccc6c6ccccc65)c(-c5ccccc5)cc4c4cc(-c5ccccc5)c(-n5c6ccccc6c6ccccc65)cc43)cc2)cc1 ; CC(C)(C)c1ccc2c(c1)c1ccc(-n3c4ccccc4c4ccccc43)cc1n2-c1c(-c2ccc(-c3ccccc3)cc2)cc(-c2nc(-c3ccccc3)nc(-c3ccccc3)n2)cc1-c1ccc(-c2ccccc2)cc1]

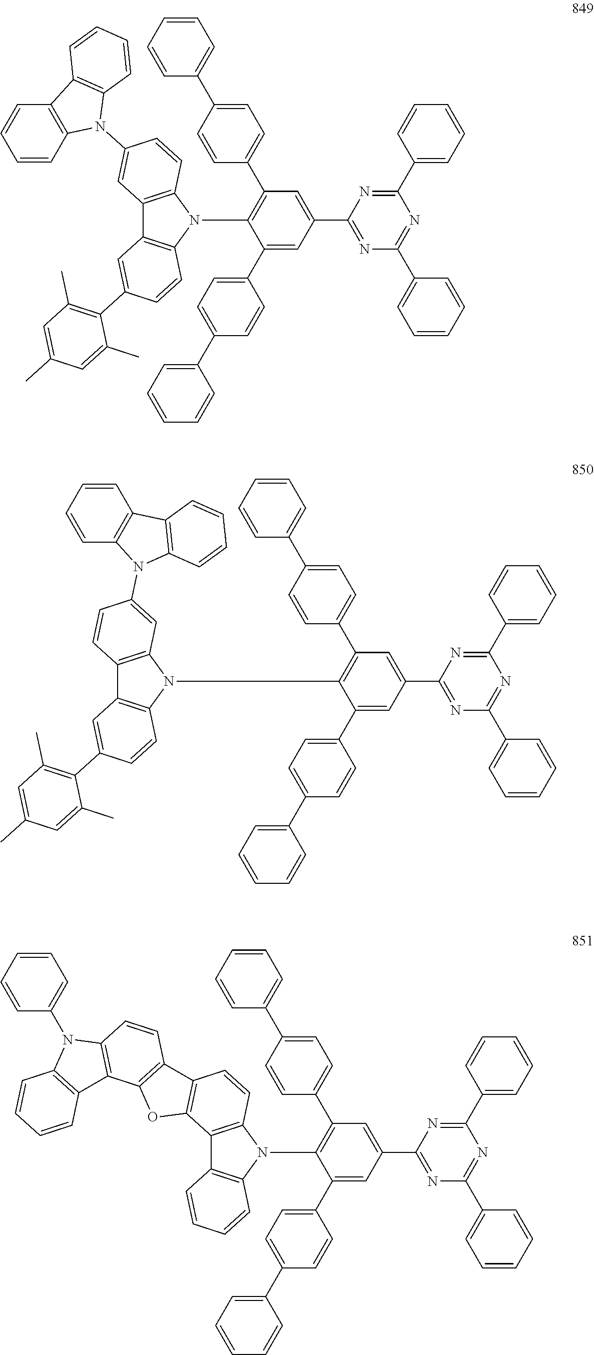 [SMILES: Cc1cc(C)c(-c2ccc3c(c2)c2cc(-n4c5ccccc5c5ccccc54)ccc2n3-c2c(-c3ccc(-c4ccccc4)cc3)cc(-c3nc(-c4ccccc4)nc(-c4ccccc4)n3)cc2-c2ccc(-c3ccccc3)cc2)c(C)c1 ; Cc1cc(C)c(-c2ccc3c(c2)c2ccc(-n4c5ccccc5c5ccccc54)cc2n3-c2c(-c3ccc(-c4ccccc4)cc3)cc(-c3nc(-c4ccccc4)nc(-c4ccccc4)n3)cc2-c2ccc(-c3ccccc3)cc2)c(C)c1 ; c1ccc(-c2ccc(-c3cc(-c4nc(-c5ccccc5)nc(-c5ccccc5)n4)cc(-c4ccc(-c5ccccc5)cc4)c3-n3c4ccccc4c4c5oc6c(ccc7c6c6ccccc6n7-c6ccccc6)c5ccc43)cc2)cc1]

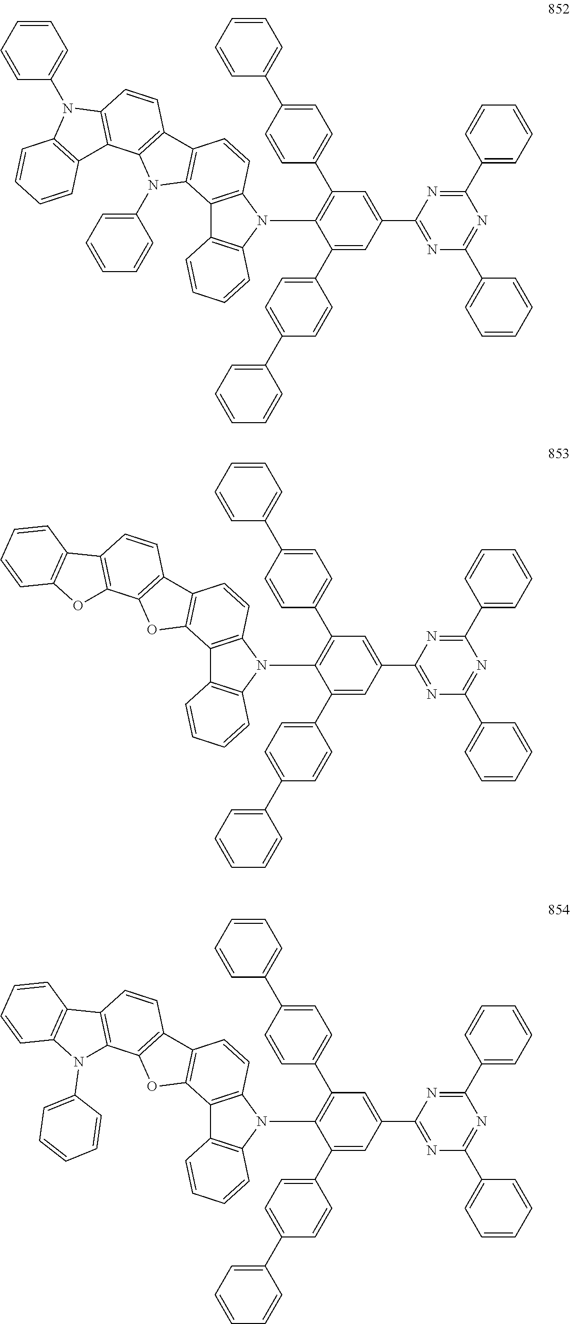

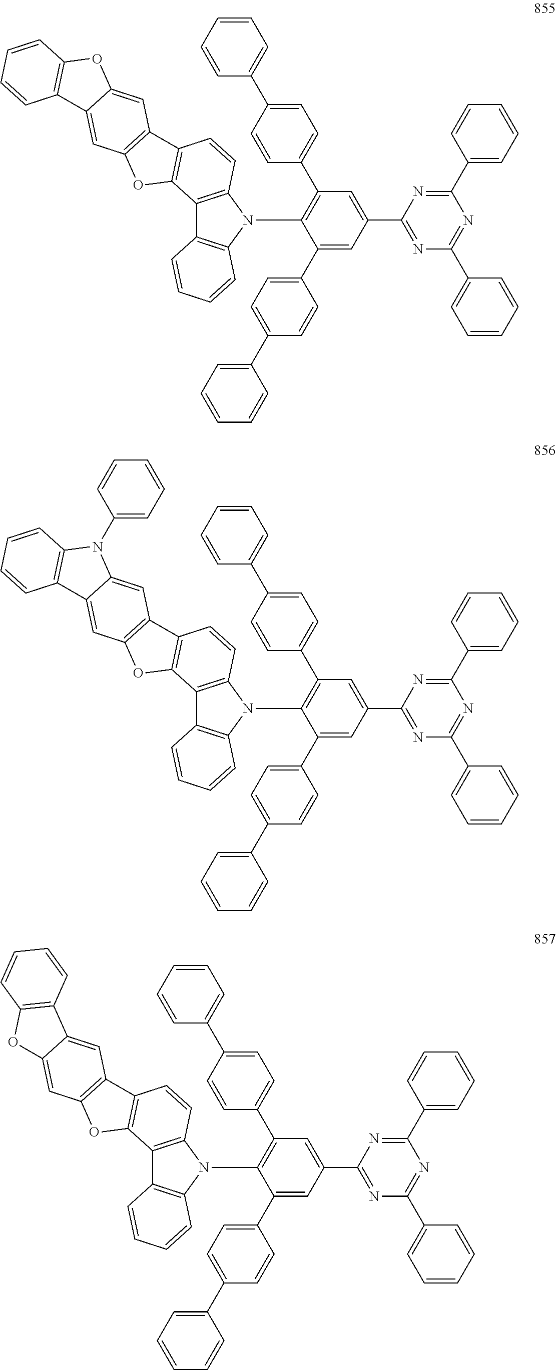 [SMILES: c1ccc(-c2ccc(-c3cc(-c4nc(-c5ccccc5)nc(-c5ccccc5)n4)cc(-c4ccc(-c5ccccc5)cc4)c3-n3c4ccccc4c4c5oc6cc7c(cc6c5ccc43)oc3ccccc37)cc2)cc1 ; c1ccc(-c2ccc(-c3cc(-c4nc(-c5ccccc5)nc(-c5ccccc5)n4)cc(-c4ccc(-c5ccccc5)cc4)c3-n3c4ccccc4c4c5oc6cc7c8ccccc8n(-c8ccccc8)c7cc6c5ccc43)cc2)cc1 ; c1ccc(-c2ccc(-c3cc(-c4nc(-c5ccccc5)nc(-c5ccccc5)n4)cc(-c4ccc(-c5ccccc5)cc4)c3-n3c4ccccc4c4c5oc6cc7oc8ccccc8c7cc6c5ccc43)cc2)cc1]

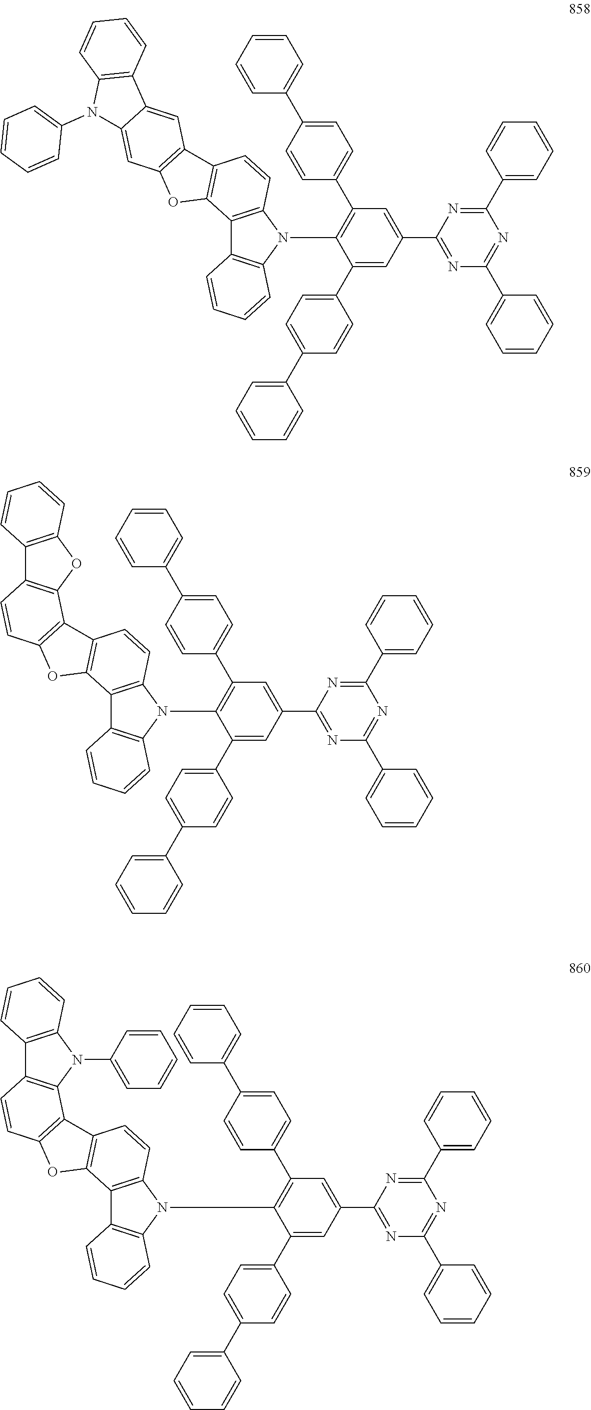 [SMILES: c1ccc(-c2ccc(-c3cc(-c4nc(-c5ccccc5)nc(-c5ccccc5)n4)cc(-c4ccc(-c5ccccc5)cc4)c3-n3c4ccccc4c4c5oc6cc7c(cc6c5ccc43)c3ccccc3n7-c3ccccc3)cc2)cc1 ; c1ccc(-c2ccc(-c3cc(-c4nc(-c5ccccc5)nc(-c5ccccc5)n4)cc(-c4ccc(-c5ccccc5)cc4)c3-n3c4ccccc4c4c5oc6ccc7c8ccccc8oc7c6c5ccc43)cc2)cc1 ; c1ccc(-c2ccc(-c3cc(-c4nc(-c5ccccc5)nc(-c5ccccc5)n4)cc(-c4ccc(-c5ccccc5)cc4)c3-n3c4ccccc4c4c5oc6ccc7c8ccccc8n(-c8ccccc8)c7c6c5ccc43)cc2)cc1]

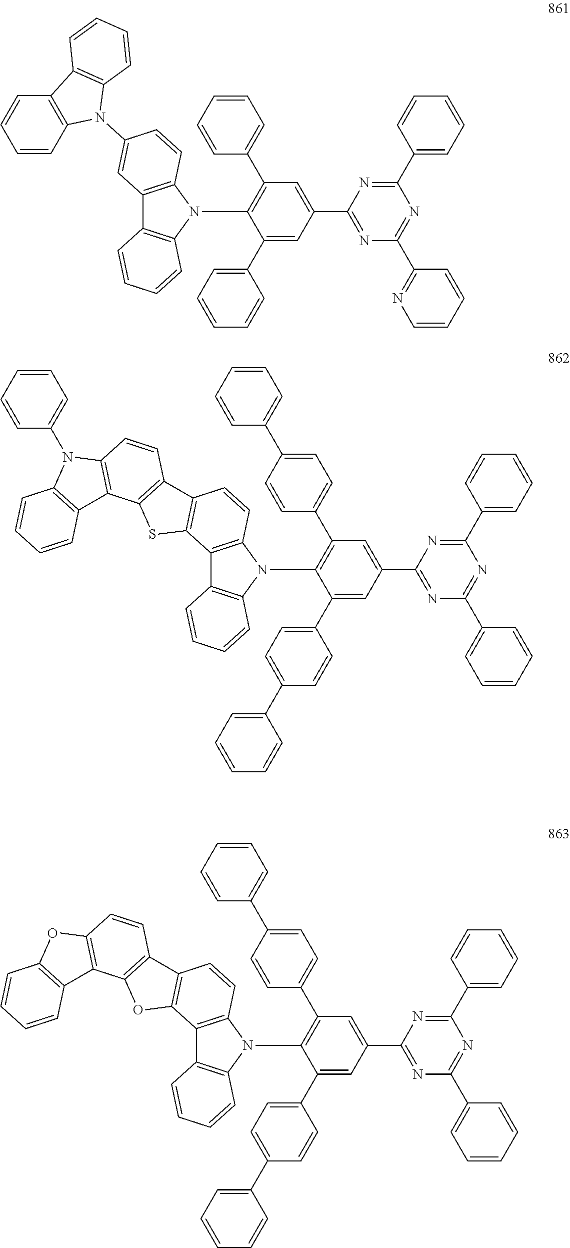

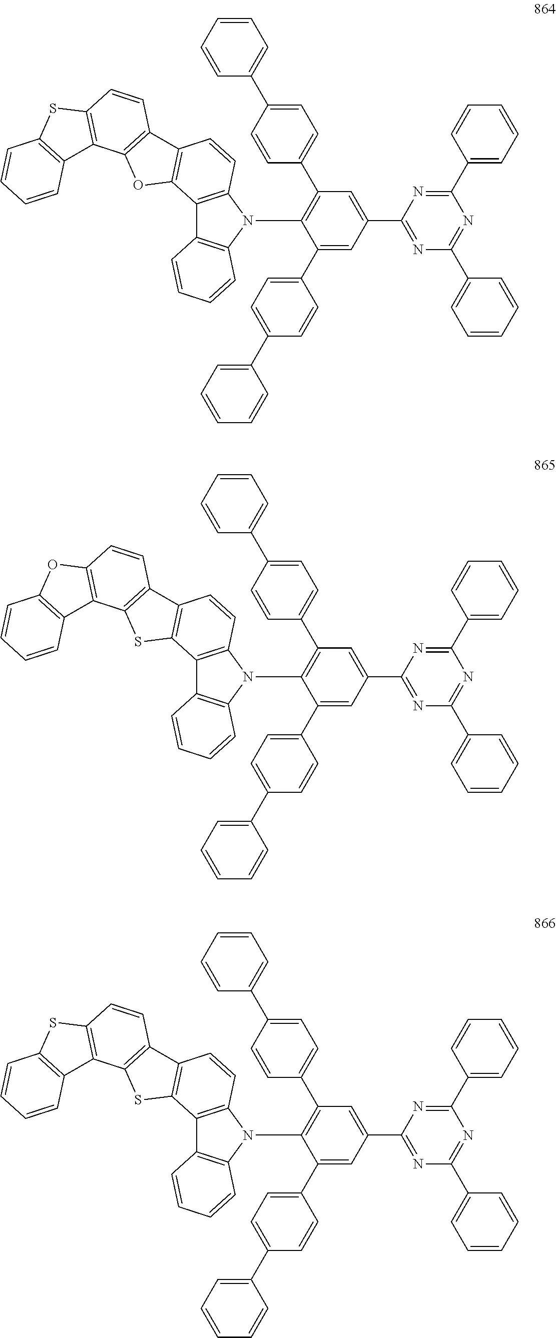 [SMILES: c1ccc(-c2ccc(-c3cc(-c4nc(-c5ccccc5)nc(-c5ccccc5)n4)cc(-c4ccc(-c5ccccc5)cc4)c3-n3c4ccccc4c4c5oc6c(ccc7sc8ccccc8c76)c5ccc43)cc2)cc1 ; c1ccc(-c2ccc(-c3cc(-c4nc(-c5ccccc5)nc(-c5ccccc5)n4)cc(-c4ccc(-c5ccccc5)cc4)c3-n3c4ccccc4c4c5sc6c(ccc7oc8ccccc8c76)c5ccc43)cc2)cc1 ; c1ccc(-c2ccc(-c3cc(-c4nc(-c5ccccc5)nc(-c5ccccc5)n4)cc(-c4ccc(-c5ccccc5)cc4)c3-n3c4ccccc4c4c5sc6c(ccc7sc8ccccc8c76)c5ccc43)cc2)cc1]

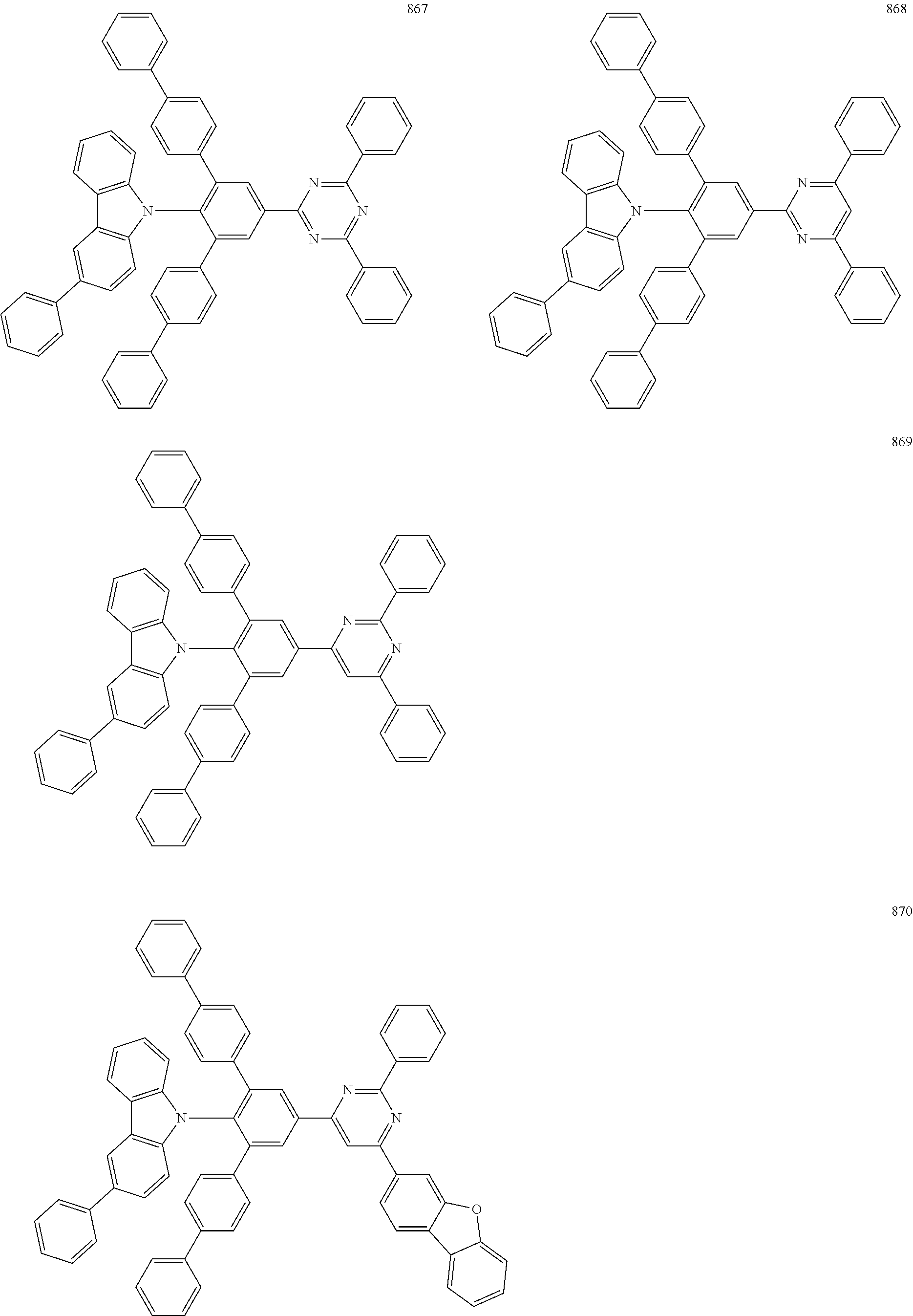

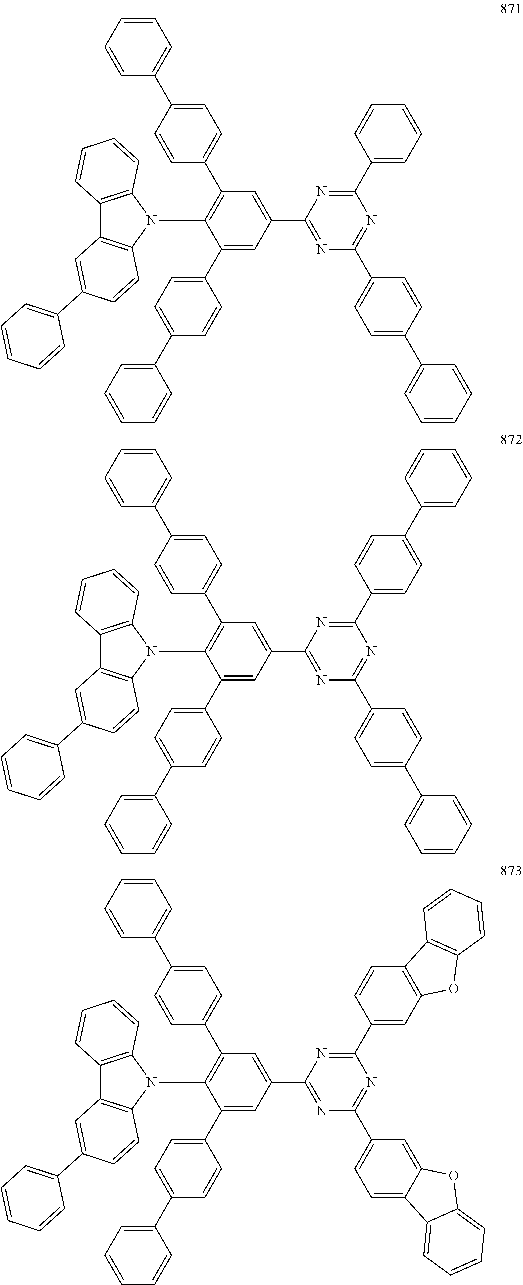

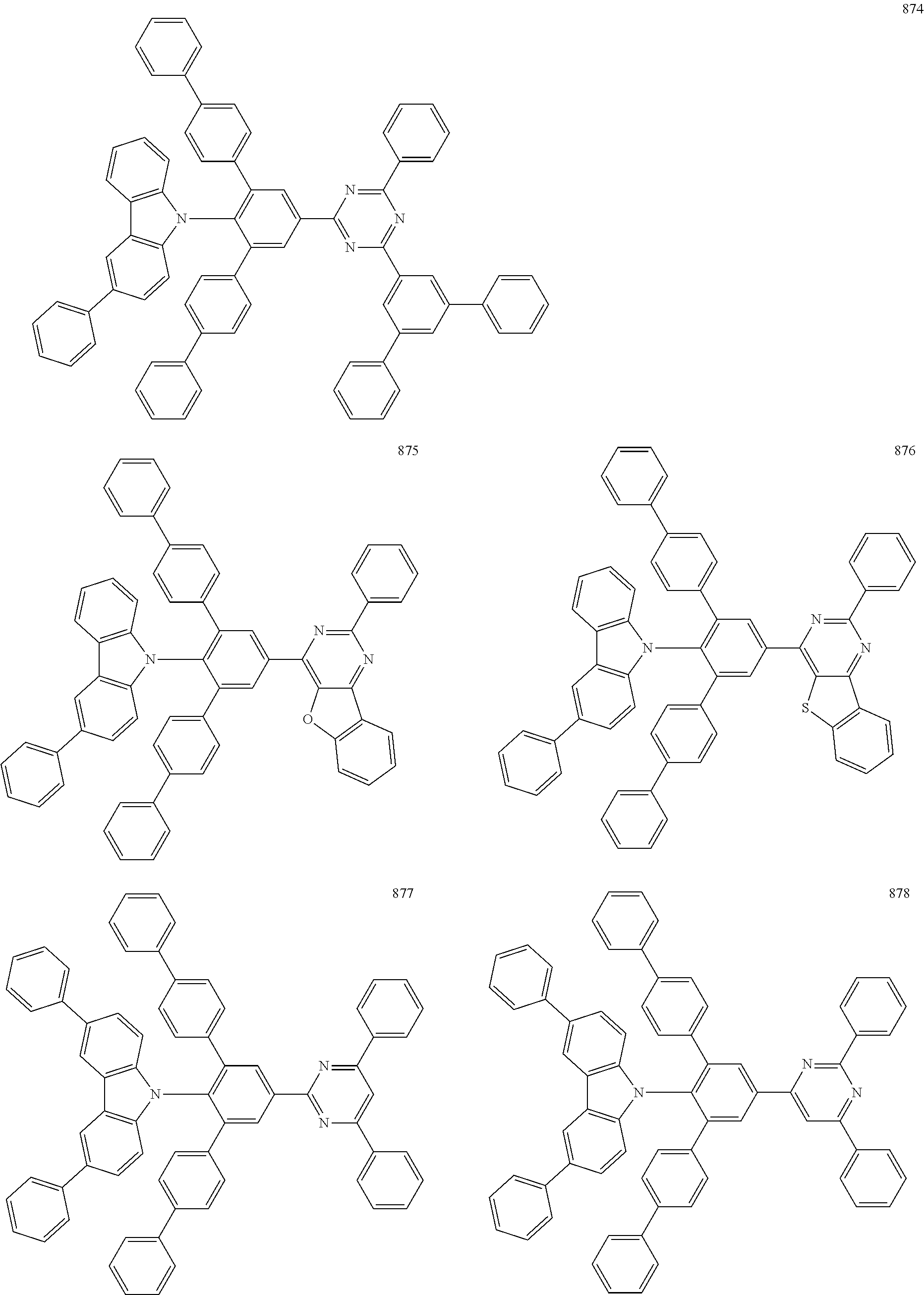

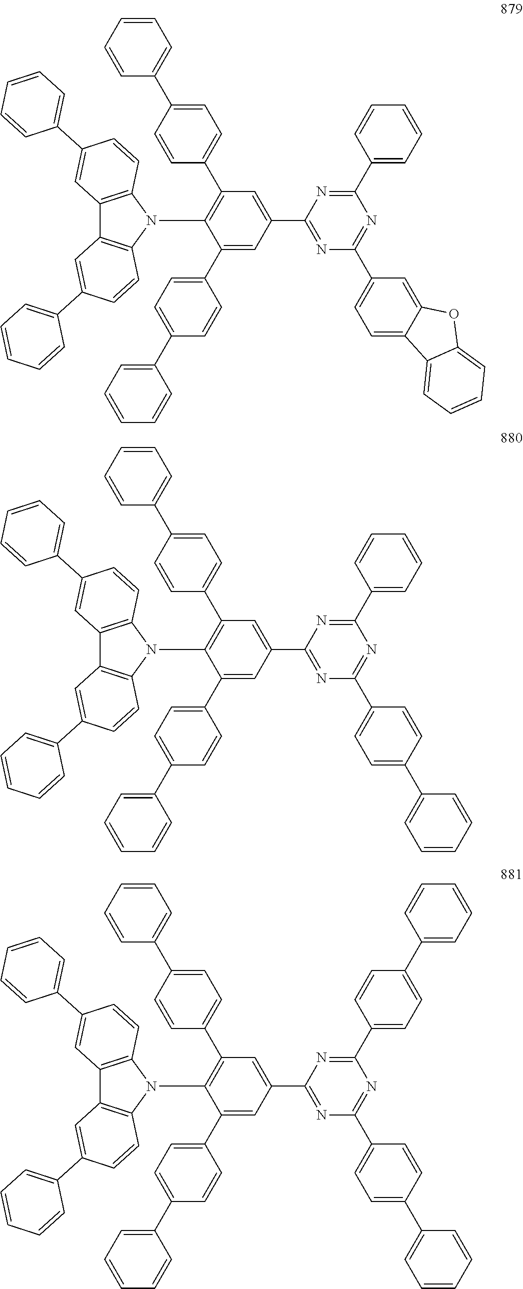

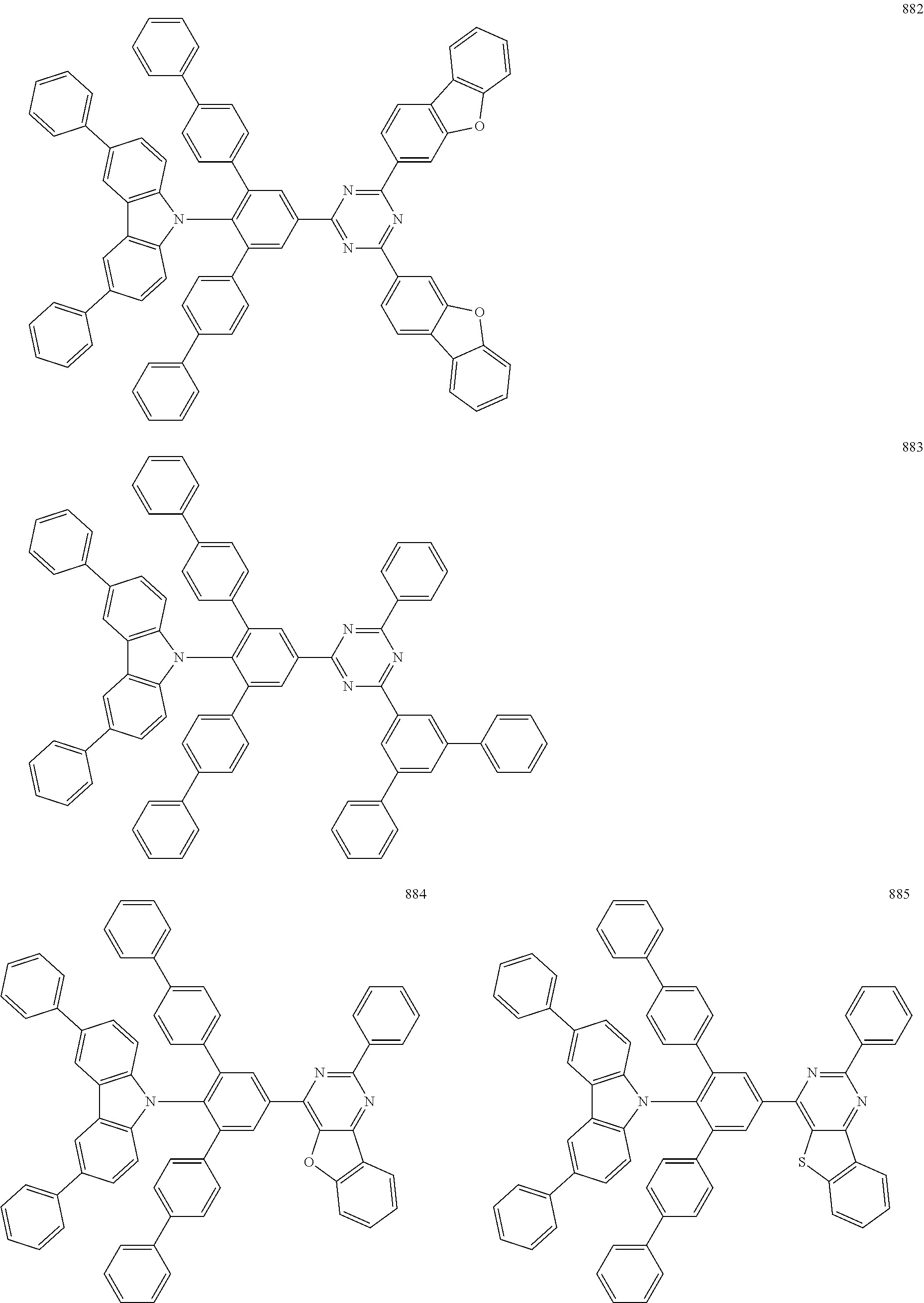

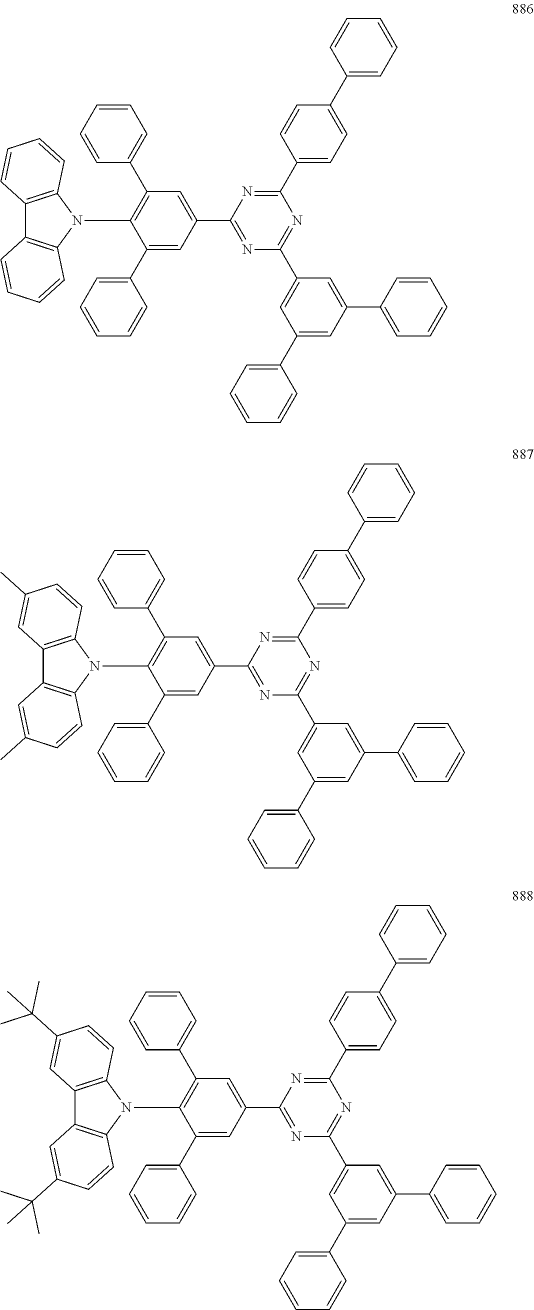 [SMILES: c1ccc(-c2ccc(-c3nc(-c4cc(-c5ccccc5)cc(-c5ccccc5)c4)nc(-c4cc(-c5ccccc5)c(-n5c6ccccc6c6ccccc65)c(-c5ccccc5)c4)n3)cc2)cc1 ; Cc1ccc2c(c1)c1cc(C)ccc1n2-c1c(-c2ccccc2)cc(-c2nc(-c3ccc(-c4ccccc4)cc3)nc(-c3cc(-c4ccccc4)cc(-c4ccccc4)c3)n2)cc1-c1ccccc1 ; CC(C)(C)c1ccc2c(c1)c1cc(C(C)(C)C)ccc1n2-c1c(-c2ccccc2)cc(-c2nc(-c3ccc(-c4ccccc4)cc3)nc(-c3cc(-c4ccccc4)cc(-c4ccccc4)c3)n2)cc1-c1ccccc1]

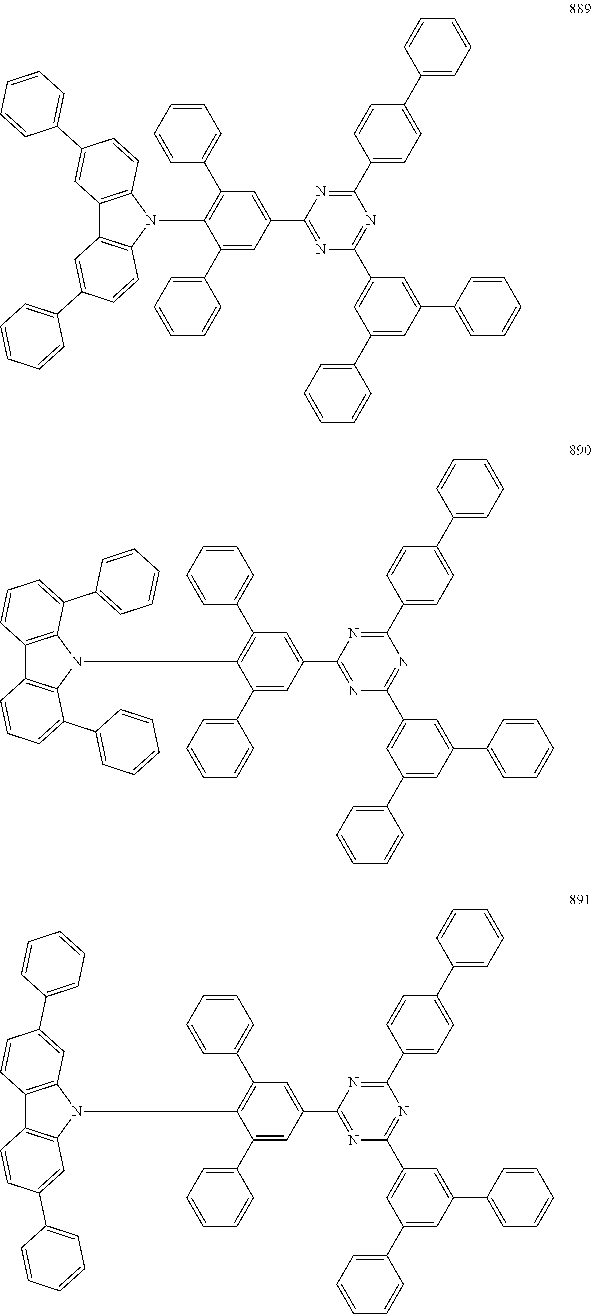 [SMILES: c1ccc(-c2ccc(-c3nc(-c4cc(-c5ccccc5)cc(-c5ccccc5)c4)nc(-c4cc(-c5ccccc5)c(-n5c6ccc(-c7ccccc7)cc6c6cc(-c7ccccc7)ccc65)c(-c5ccccc5)c4)n3)cc2)cc1 ; c1ccc(-c2ccc(-c3nc(-c4cc(-c5ccccc5)cc(-c5ccccc5)c4)nc(-c4cc(-c5ccccc5)c(-n5c6c(-c7ccccc7)cccc6c6cccc(-c7ccccc7)c65)c(-c5ccccc5)c4)n3)cc2)cc1 ; c1ccc(-c2ccc(-c3nc(-c4cc(-c5ccccc5)cc(-c5ccccc5)c4)nc(-c4cc(-c5ccccc5)c(-n5c6cc(-c7ccccc7)ccc6c6ccc(-c7ccccc7)cc65)c(-c5ccccc5)c4)n3)cc2)cc1]

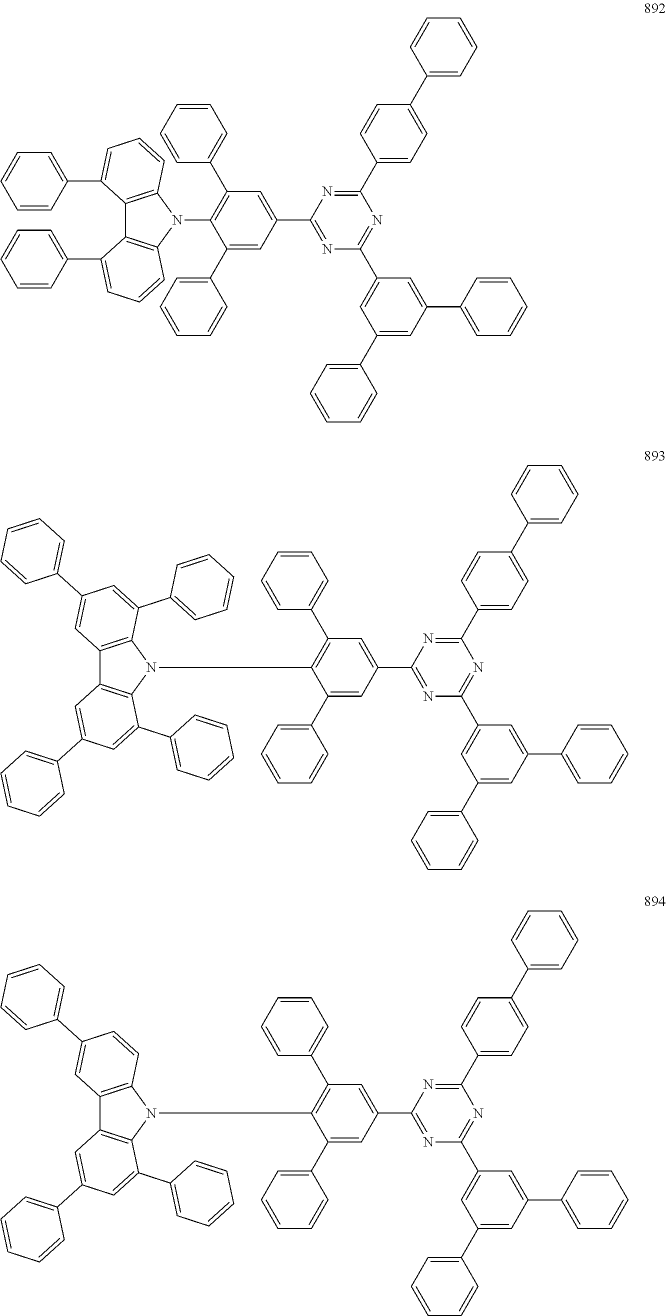 [SMILES: c1ccc(-c2ccc(-c3nc(-c4cc(-c5ccccc5)cc(-c5ccccc5)c4)nc(-c4cc(-c5ccccc5)c(-n5c6cccc(-c7ccccc7)c6c6c(-c7ccccc7)cccc65)c(-c5ccccc5)c4)n3)cc2)cc1 ; c1ccc(-c2ccc(-c3nc(-c4cc(-c5ccccc5)cc(-c5ccccc5)c4)nc(-c4cc(-c5ccccc5)c(-n5c6c(-c7ccccc7)cc(-c7ccccc7)cc6c6cc(-c7ccccc7)cc(-c7ccccc7)c65)c(-c5ccccc5)c4)n3)cc2)cc1 ; c1ccc(-c2ccc(-c3nc(-c4cc(-c5ccccc5)cc(-c5ccccc5)c4)nc(-c4cc(-c5ccccc5)c(-n5c6ccc(-c7ccccc7)cc6c6cc(-c7ccccc7)cc(-c7ccccc7)c65)c(-c5ccccc5)c4)n3)cc2)cc1]

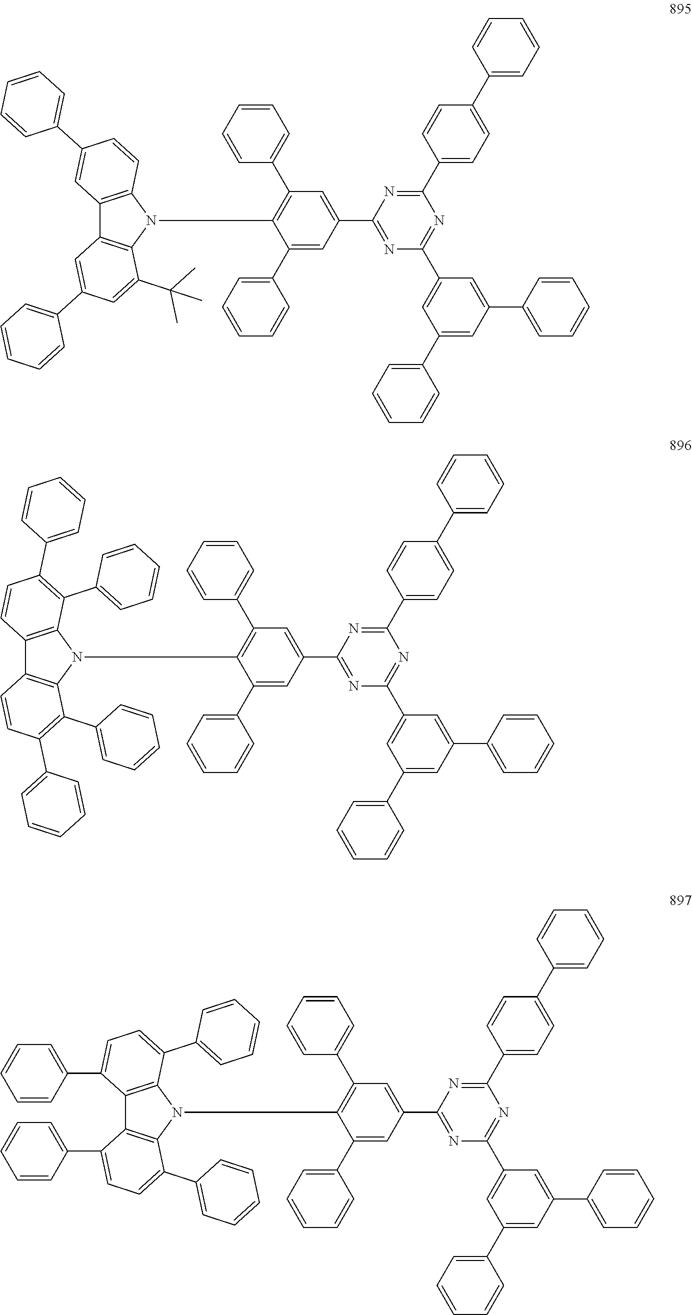

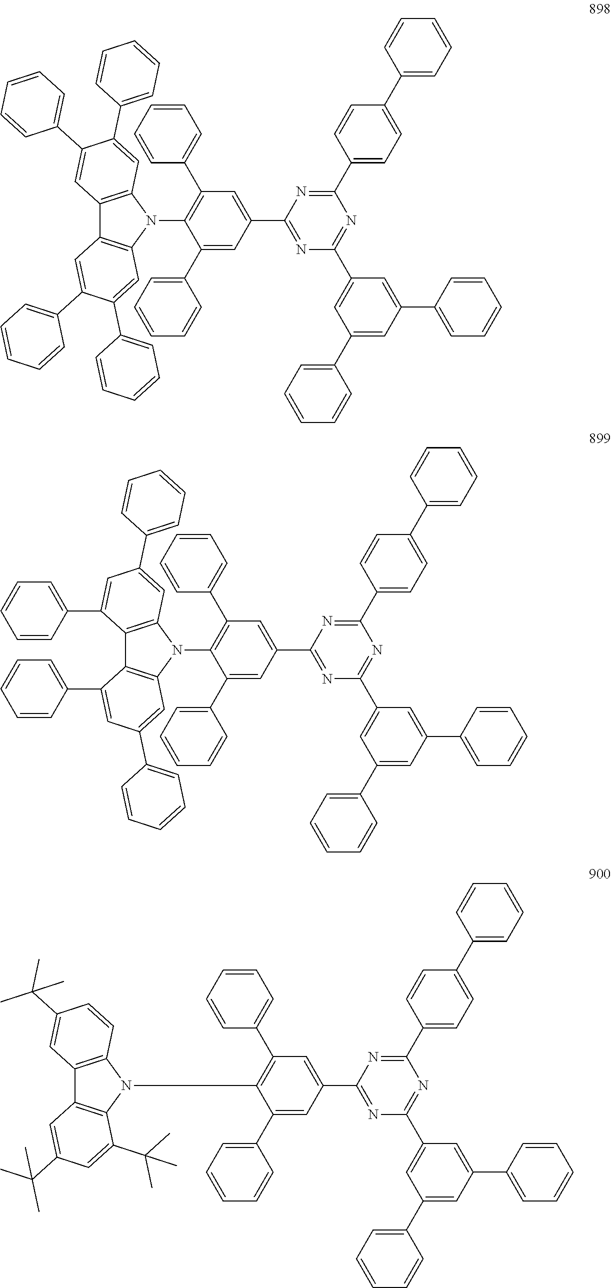 [SMILES: c1ccc(-c2ccc(-c3nc(-c4cc(-c5ccccc5)cc(-c5ccccc5)c4)nc(-c4cc(-c5ccccc5)c(-n5c6cc(-c7ccccc7)c(-c7ccccc7)cc6c6cc(-c7ccccc7)c(-c7ccccc7)cc65)c(-c5ccccc5)c4)n3)cc2)cc1 ; c1ccc(-c2ccc(-c3nc(-c4cc(-c5ccccc5)cc(-c5ccccc5)c4)nc(-c4cc(-c5ccccc5)c(-n5c6cc(-c7ccccc7)cc(-c7ccccc7)c6c6c(-c7ccccc7)cc(-c7ccccc7)cc65)c(-c5ccccc5)c4)n3)cc2)cc1 ; CC(C)(C)c1ccc2c(c1)c1cc(C(C)(C)C)cc(C(C)(C)C)c1n2-c1c(-c2ccccc2)cc(-c2nc(-c3ccc(-c4ccccc4)cc3)nc(-c3cc(-c4ccccc4)cc(-c4ccccc4)c3)n2)cc1-c1ccccc1]

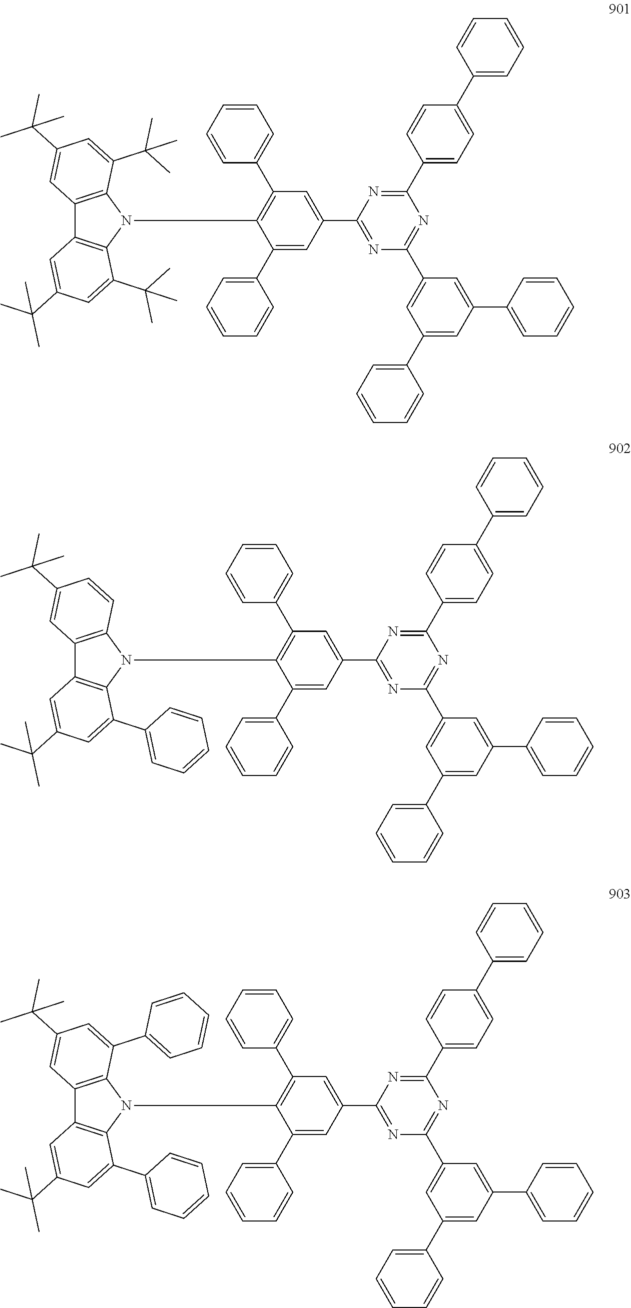 [SMILES: CC(C)(C)c1cc(C(C)(C)C)c2c(c1)c1cc(C(C)(C)C)cc(C(C)(C)C)c1n2-c1c(-c2ccccc2)cc(-c2nc(-c3ccc(-c4ccccc4)cc3)nc(-c3cc(-c4ccccc4)cc(-c4ccccc4)c3)n2)cc1-c1ccccc1 ; CC(C)(C)c1ccc2c(c1)c1cc(C(C)(C)C)cc(-c3ccccc3)c1n2-c1c(-c2ccccc2)cc(-c2nc(-c3ccc(-c4ccccc4)cc3)nc(-c3cc(-c4ccccc4)cc(-c4ccccc4)c3)n2)cc1-c1ccccc1 ; CC(C)(C)c1cc(-c2ccccc2)c2c(c1)c1cc(C(C)(C)C)cc(-c3ccccc3)c1n2-c1c(-c2ccccc2)cc(-c2nc(-c3ccc(-c4ccccc4)cc3)nc(-c3cc(-c4ccccc4)cc(-c4ccccc4)c3)n2)cc1-c1ccccc1]

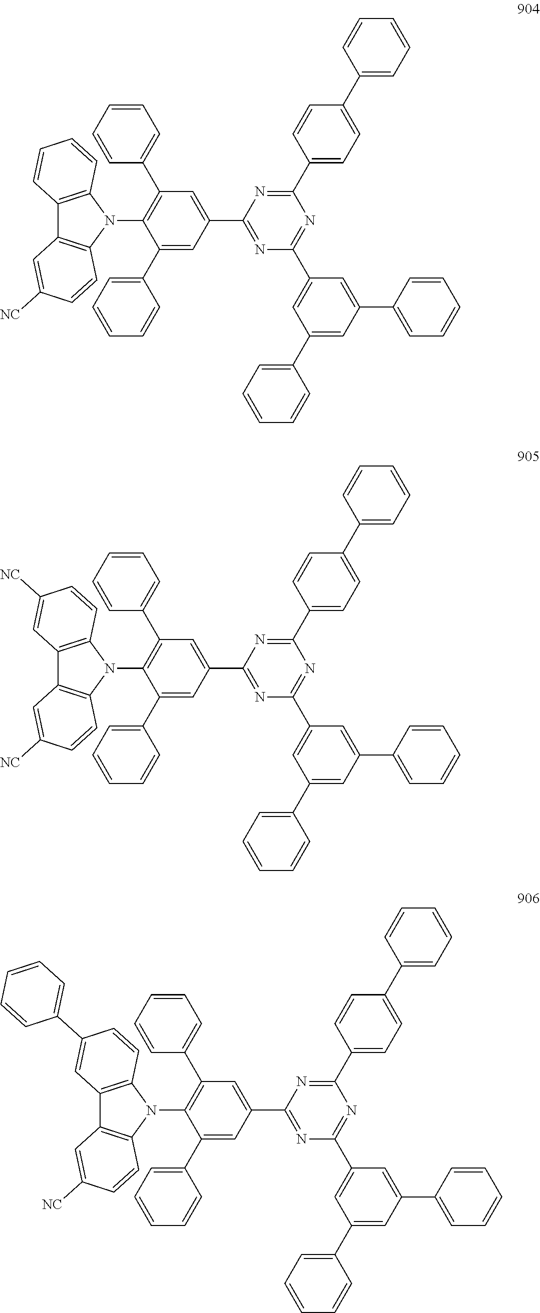 [SMILES: N#Cc1ccc2c(c1)c1ccccc1n2-c1c(-c2ccccc2)cc(-c2nc(-c3ccc(-c4ccccc4)cc3)nc(-c3cc(-c4ccccc4)cc(-c4ccccc4)c3)n2)cc1-c1ccccc1 ; N#Cc1ccc2c(c1)c1cc(C#N)ccc1n2-c1c(-c2ccccc2)cc(-c2nc(-c3ccc(-c4ccccc4)cc3)nc(-c3cc(-c4ccccc4)cc(-c4ccccc4)c3)n2)cc1-c1ccccc1 ; N#Cc1ccc2c(c1)c1cc(-c3ccccc3)ccc1n2-c1c(-c2ccccc2)cc(-c2nc(-c3ccc(-c4ccccc4)cc3)nc(-c3cc(-c4ccccc4)cc(-c4ccccc4)c3)n2)cc1-c1ccccc1]

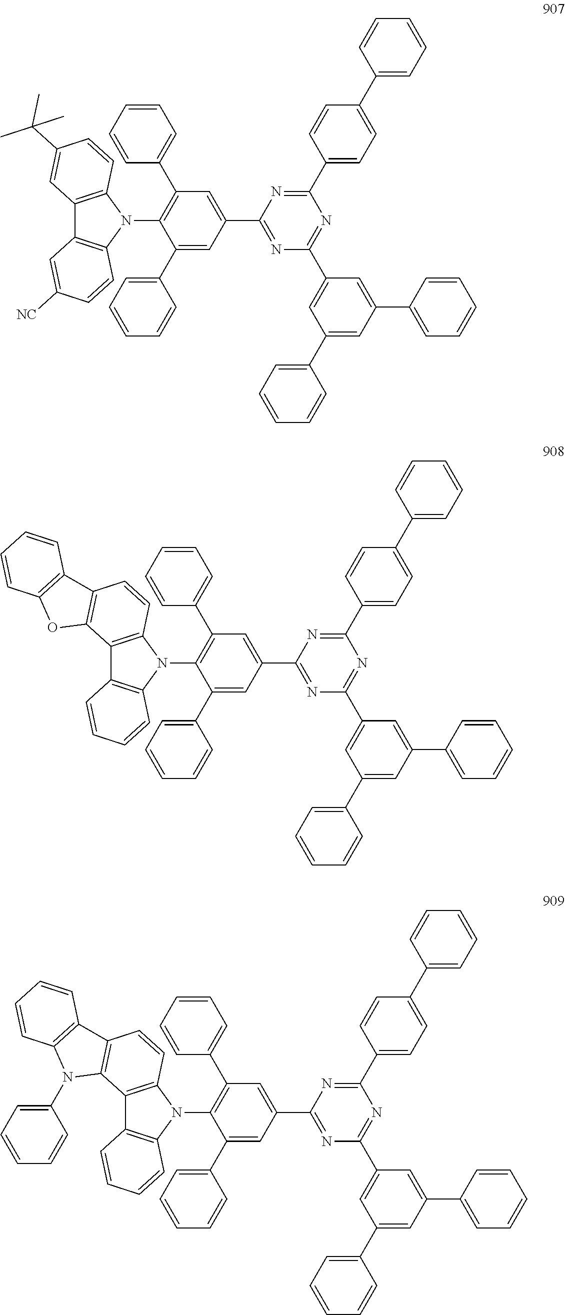 [SMILES: CC(C)(C)c1ccc2c(c1)c1cc(C#N)ccc1n2-c1c(-c2ccccc2)cc(-c2nc(-c3ccc(-c4ccccc4)cc3)nc(-c3cc(-c4ccccc4)cc(-c4ccccc4)c3)n2)cc1-c1ccccc1 ; c1ccc(-c2ccc(-c3nc(-c4cc(-c5ccccc5)cc(-c5ccccc5)c4)nc(-c4cc(-c5ccccc5)c(-n5c6ccccc6c6c7oc8ccccc8c7ccc65)c(-c5ccccc5)c4)n3)cc2)cc1 ; c1ccc(-c2ccc(-c3nc(-c4cc(-c5ccccc5)cc(-c5ccccc5)c4)nc(-c4cc(-c5ccccc5)c(-n5c6ccccc6c6c5ccc5c7ccccc7n(-c7ccccc7)c56)c(-c5ccccc5)c4)n3)cc2)cc1]

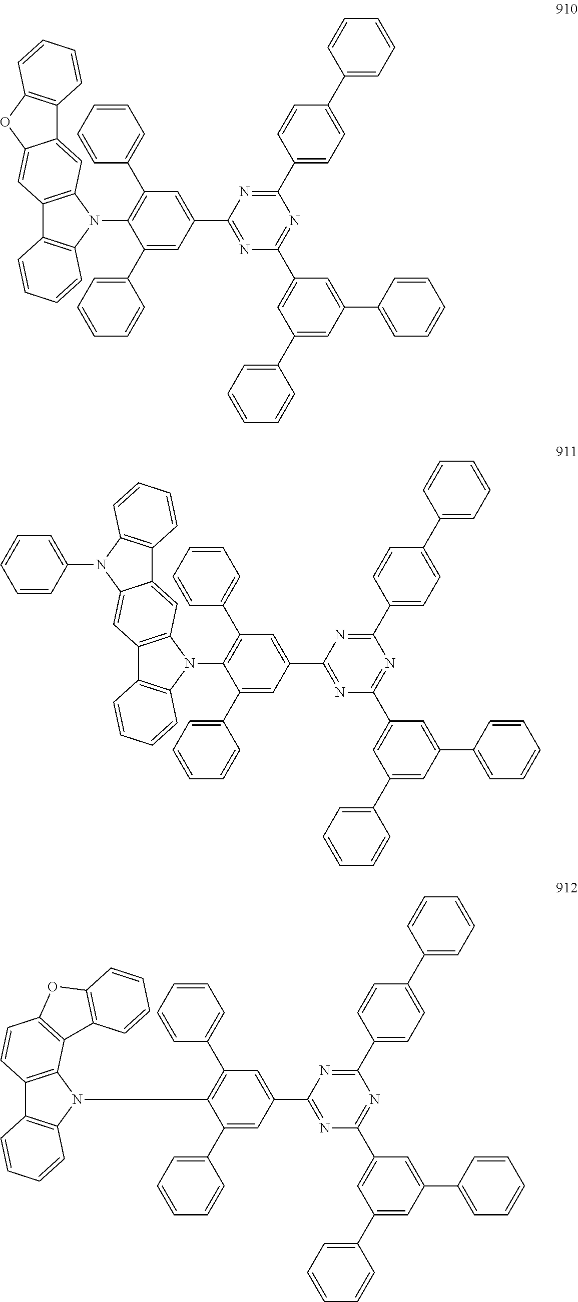 [SMILES: c1ccc(-c2ccc(-c3nc(-c4cc(-c5ccccc5)cc(-c5ccccc5)c4)nc(-c4cc(-c5ccccc5)c(-n5c6ccccc6c6cc7oc8ccccc8c7cc65)c(-c5ccccc5)c4)n3)cc2)cc1 ; c1ccc(-c2ccc(-c3nc(-c4cc(-c5ccccc5)cc(-c5ccccc5)c4)nc(-c4cc(-c5ccccc5)c(-n5c6ccccc6c6cc7c(cc65)c5ccccc5n7-c5ccccc5)c(-c5ccccc5)c4)n3)cc2)cc1 ; c1ccc(-c2ccc(-c3nc(-c4cc(-c5ccccc5)cc(-c5ccccc5)c4)nc(-c4cc(-c5ccccc5)c(-n5c6ccccc6c6ccc7oc8ccccc8c7c65)c(-c5ccccc5)c4)n3)cc2)cc1]

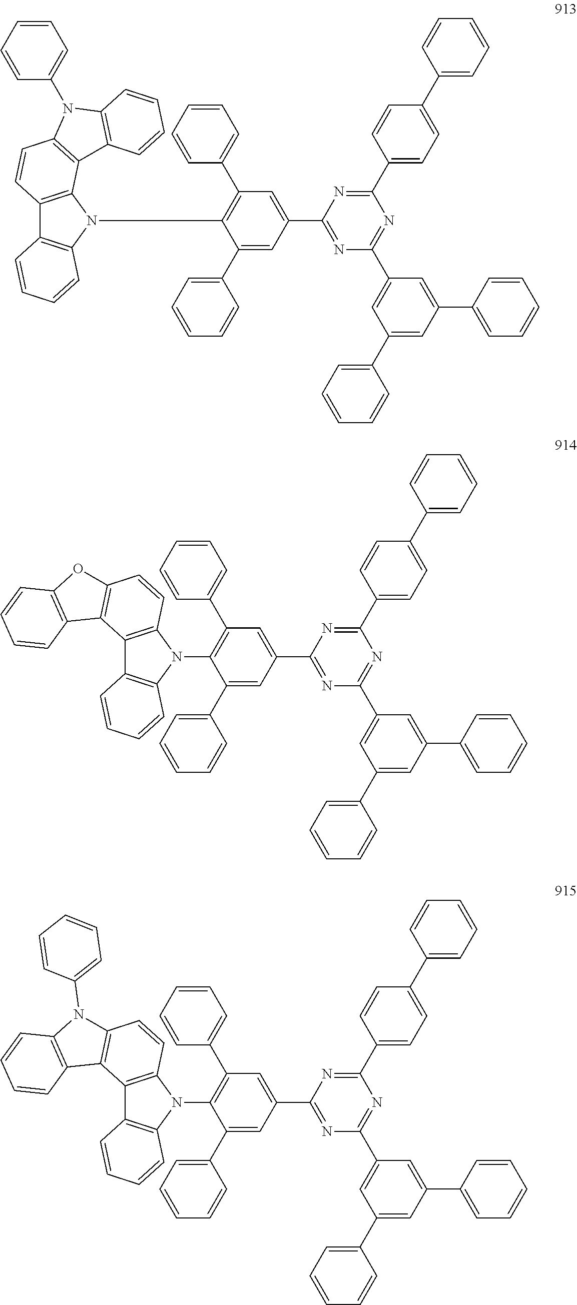 [SMILES: c1ccc(-c2ccc(-c3nc(-c4cc(-c5ccccc5)cc(-c5ccccc5)c4)nc(-c4cc(-c5ccccc5)c(-n5c6ccccc6c6ccc7c(c8ccccc8n7-c7ccccc7)c65)c(-c5ccccc5)c4)n3)cc2)cc1 ; c1ccc(-c2ccc(-c3nc(-c4cc(-c5ccccc5)cc(-c5ccccc5)c4)nc(-c4cc(-c5ccccc5)c(-n5c6ccccc6c6c7c(ccc65)oc5ccccc57)c(-c5ccccc5)c4)n3)cc2)cc1 ; c1ccc(-c2ccc(-c3nc(-c4cc(-c5ccccc5)cc(-c5ccccc5)c4)nc(-c4cc(-c5ccccc5)c(-n5c6ccccc6c6c7c8ccccc8n(-c8ccccc8)c7ccc65)c(-c5ccccc5)c4)n3)cc2)cc1]

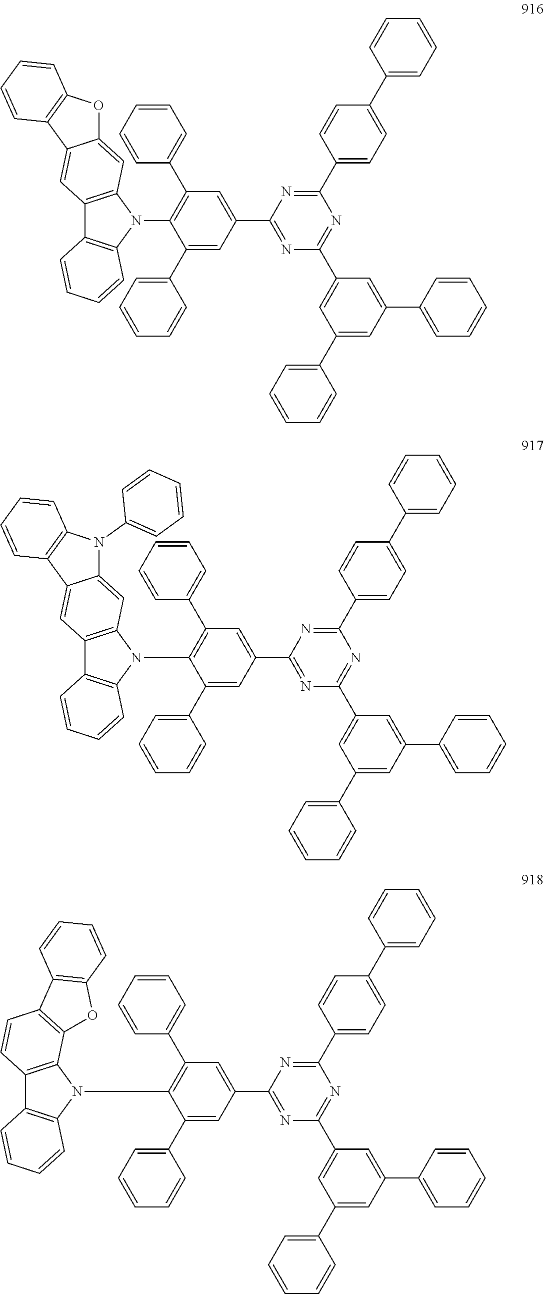 [SMILES: c1ccc(-c2ccc(-c3nc(-c4cc(-c5ccccc5)cc(-c5ccccc5)c4)nc(-c4cc(-c5ccccc5)c(-n5c6ccccc6c6cc7c(cc65)oc5ccccc57)c(-c5ccccc5)c4)n3)cc2)cc1 ; c1ccc(-c2ccc(-c3nc(-c4cc(-c5ccccc5)cc(-c5ccccc5)c4)nc(-c4cc(-c5ccccc5)c(-n5c6ccccc6c6cc7c8ccccc8n(-c8ccccc8)c7cc65)c(-c5ccccc5)c4)n3)cc2)cc1 ; c1ccc(-c2ccc(-c3nc(-c4cc(-c5ccccc5)cc(-c5ccccc5)c4)nc(-c4cc(-c5ccccc5)c(-n5c6ccccc6c6ccc7c8ccccc8oc7c65)c(-c5ccccc5)c4)n3)cc2)cc1]

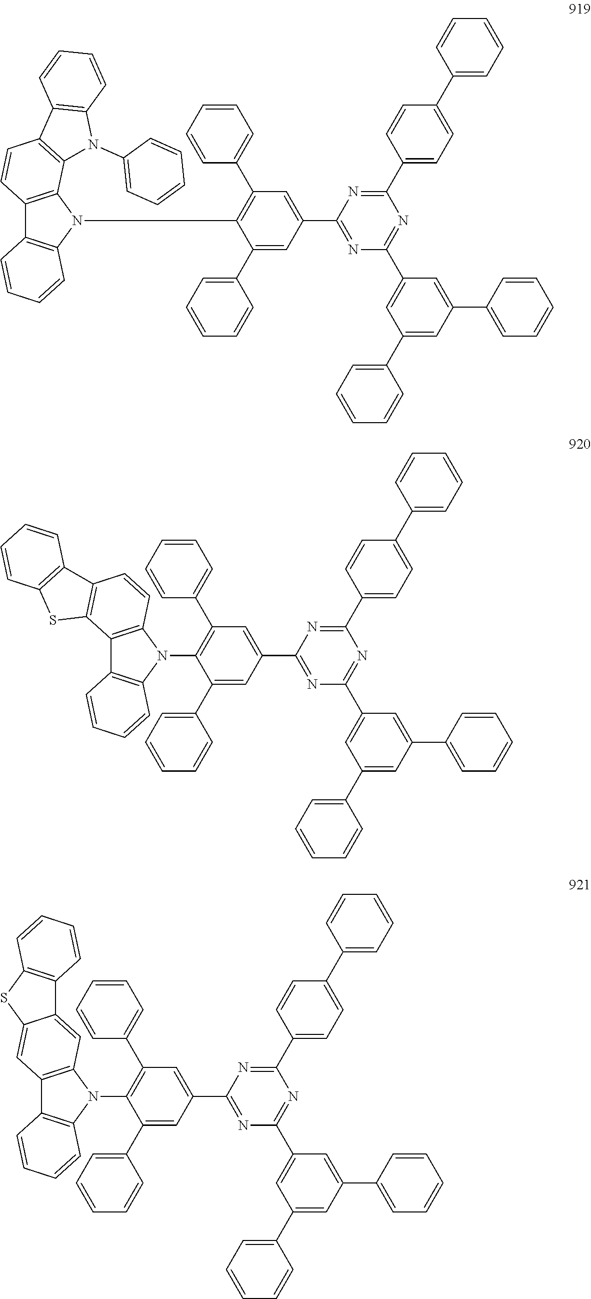

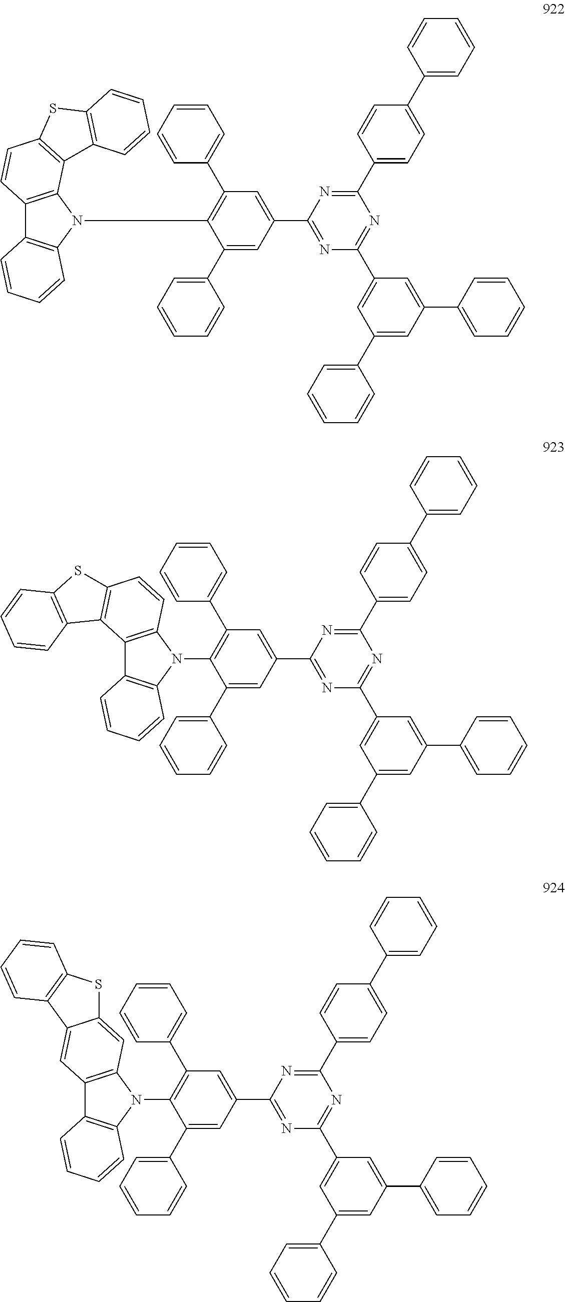

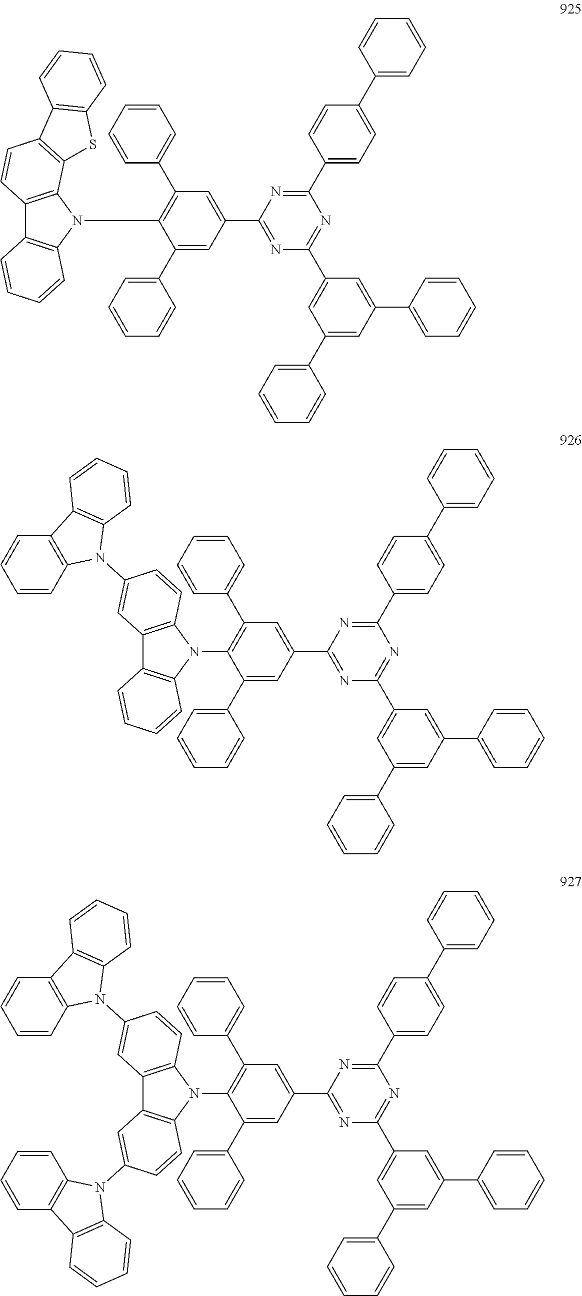 [SMILES: c1ccc(-c2ccc(-c3nc(-c4cc(-c5ccccc5)cc(-c5ccccc5)c4)nc(-c4cc(-c5ccccc5)c(-n5c6ccccc6c6ccc7c8ccccc8sc7c65)c(-c5ccccc5)c4)n3)cc2)cc1 ; c1ccc(-c2ccc(-c3nc(-c4cc(-c5ccccc5)cc(-c5ccccc5)c4)nc(-c4cc(-c5ccccc5)c(-n5c6ccccc6c6cc(-n7c8ccccc8c8ccccc87)ccc65)c(-c5ccccc5)c4)n3)cc2)cc1 ; c1ccc(-c2ccc(-c3nc(-c4cc(-c5ccccc5)cc(-c5ccccc5)c4)nc(-c4cc(-c5ccccc5)c(-n5c6ccc(-n7c8ccccc8c8ccccc87)cc6c6cc(-n7c8ccccc8c8ccccc87)ccc65)c(-c5ccccc5)c4)n3)cc2)cc1]

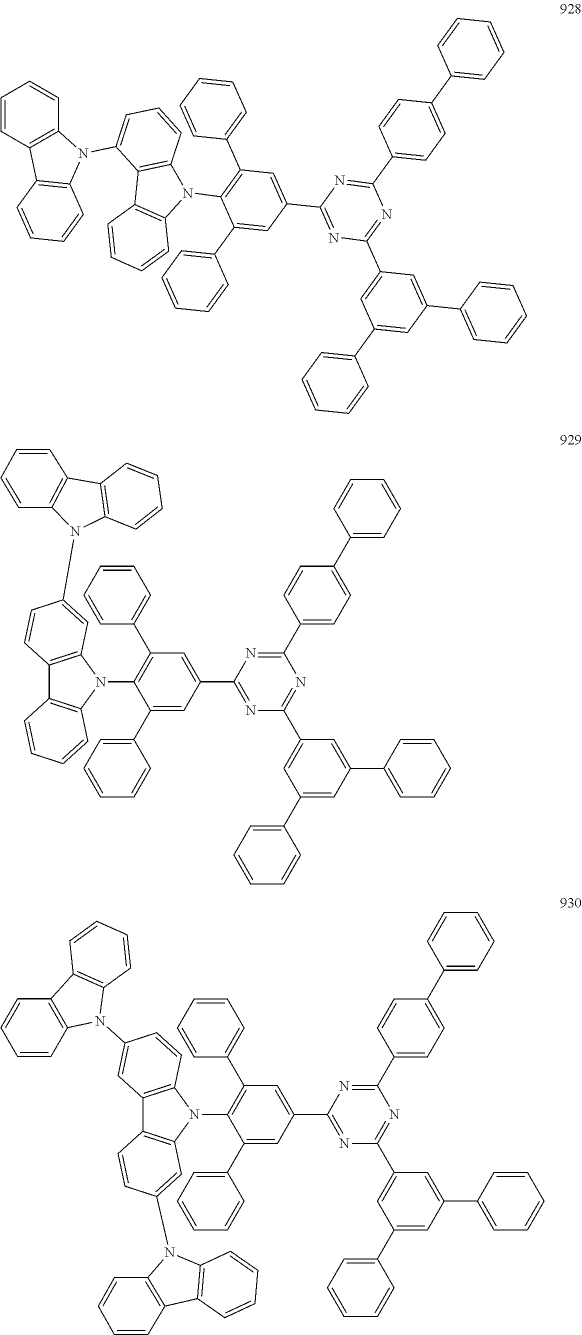 [SMILES: c1ccc(-c2ccc(-c3nc(-c4cc(-c5ccccc5)cc(-c5ccccc5)c4)nc(-c4cc(-c5ccccc5)c(-n5c6ccccc6c6c(-n7c8ccccc8c8ccccc87)cccc65)c(-c5ccccc5)c4)n3)cc2)cc1 ; c1ccc(-c2ccc(-c3nc(-c4cc(-c5ccccc5)cc(-c5ccccc5)c4)nc(-c4cc(-c5ccccc5)c(-n5c6ccccc6c6ccc(-n7c8ccccc8c8ccccc87)cc65)c(-c5ccccc5)c4)n3)cc2)cc1 ; c1ccc(-c2ccc(-c3nc(-c4cc(-c5ccccc5)cc(-c5ccccc5)c4)nc(-c4cc(-c5ccccc5)c(-n5c6ccc(-n7c8ccccc8c8ccccc87)cc6c6ccc(-n7c8ccccc8c8ccccc87)cc65)c(-c5ccccc5)c4)n3)cc2)cc1]

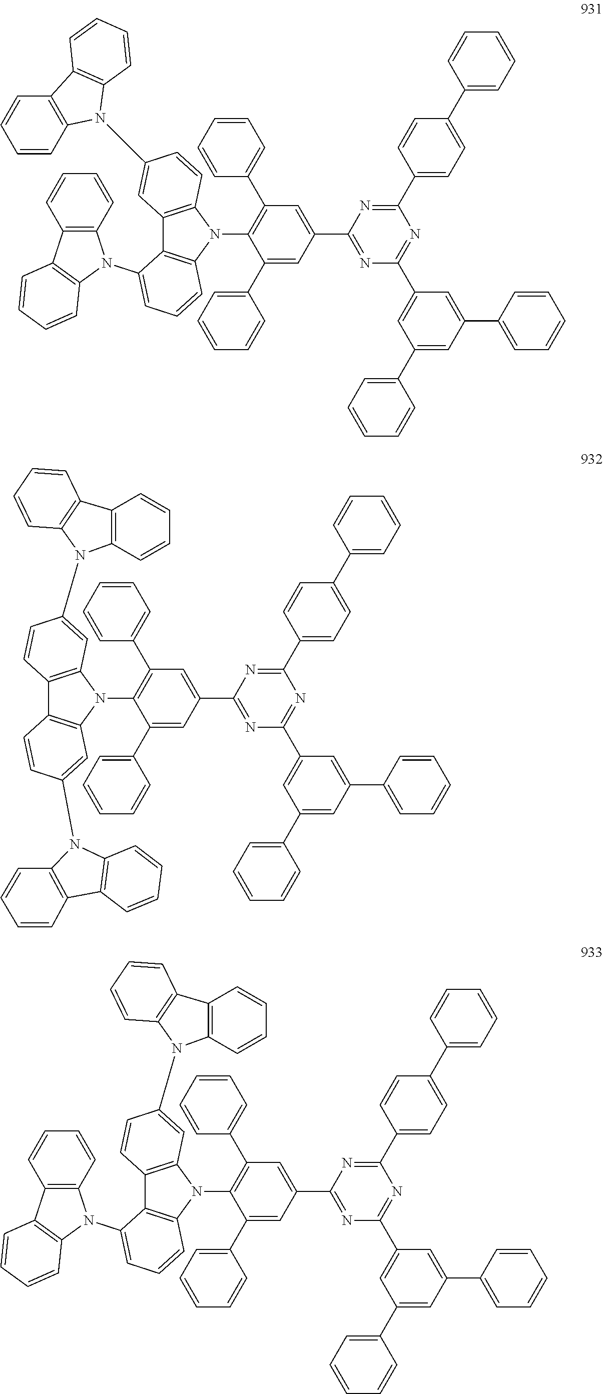 [SMILES: c1ccc(-c2ccc(-c3nc(-c4cc(-c5ccccc5)cc(-c5ccccc5)c4)nc(-c4cc(-c5ccccc5)c(-n5c6ccc(-n7c8ccccc8c8ccccc87)cc6c6c(-n7c8ccccc8c8ccccc87)cccc65)c(-c5ccccc5)c4)n3)cc2)cc1 ; c1ccc(-c2ccc(-c3nc(-c4cc(-c5ccccc5)cc(-c5ccccc5)c4)nc(-c4cc(-c5ccccc5)c(-n5c6cc(-n7c8ccccc8c8ccccc87)ccc6c6ccc(-n7c8ccccc8c8ccccc87)cc65)c(-c5ccccc5)c4)n3)cc2)cc1 ; c1ccc(-c2ccc(-c3nc(-c4cc(-c5ccccc5)cc(-c5ccccc5)c4)nc(-c4cc(-c5ccccc5)c(-n5c6cc(-n7c8ccccc8c8ccccc87)ccc6c6c(-n7c8ccccc8c8ccccc87)cccc65)c(-c5ccccc5)c4)n3)cc2)cc1]

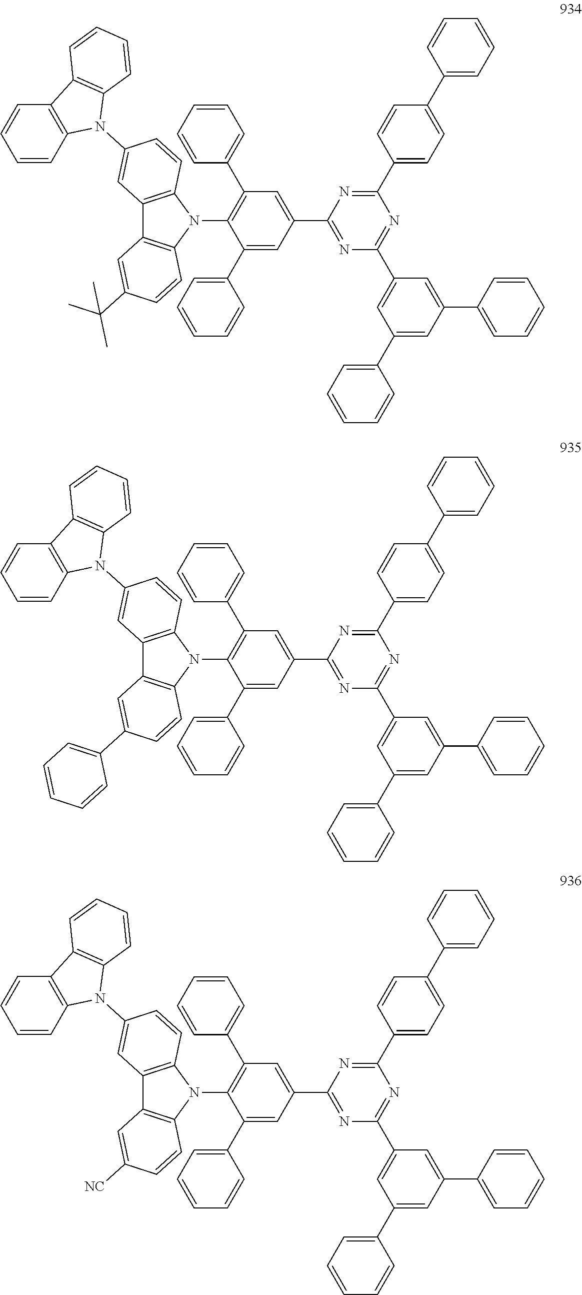 [SMILES: CC(C)(C)c1ccc2c(c1)c1cc(-n3c4ccccc4c4ccccc43)ccc1n2-c1c(-c2ccccc2)cc(-c2nc(-c3ccc(-c4ccccc4)cc3)nc(-c3cc(-c4ccccc4)cc(-c4ccccc4)c3)n2)cc1-c1ccccc1 ; c1ccc(-c2ccc(-c3nc(-c4cc(-c5ccccc5)cc(-c5ccccc5)c4)nc(-c4cc(-c5ccccc5)c(-n5c6ccc(-c7ccccc7)cc6c6cc(-n7c8ccccc8c8ccccc87)ccc65)c(-c5ccccc5)c4)n3)cc2)cc1 ; N#Cc1ccc2c(c1)c1cc(-n3c4ccccc4c4ccccc43)ccc1n2-c1c(-c2ccccc2)cc(-c2nc(-c3ccc(-c4ccccc4)cc3)nc(-c3cc(-c4ccccc4)cc(-c4ccccc4)c3)n2)cc1-c1ccccc1]

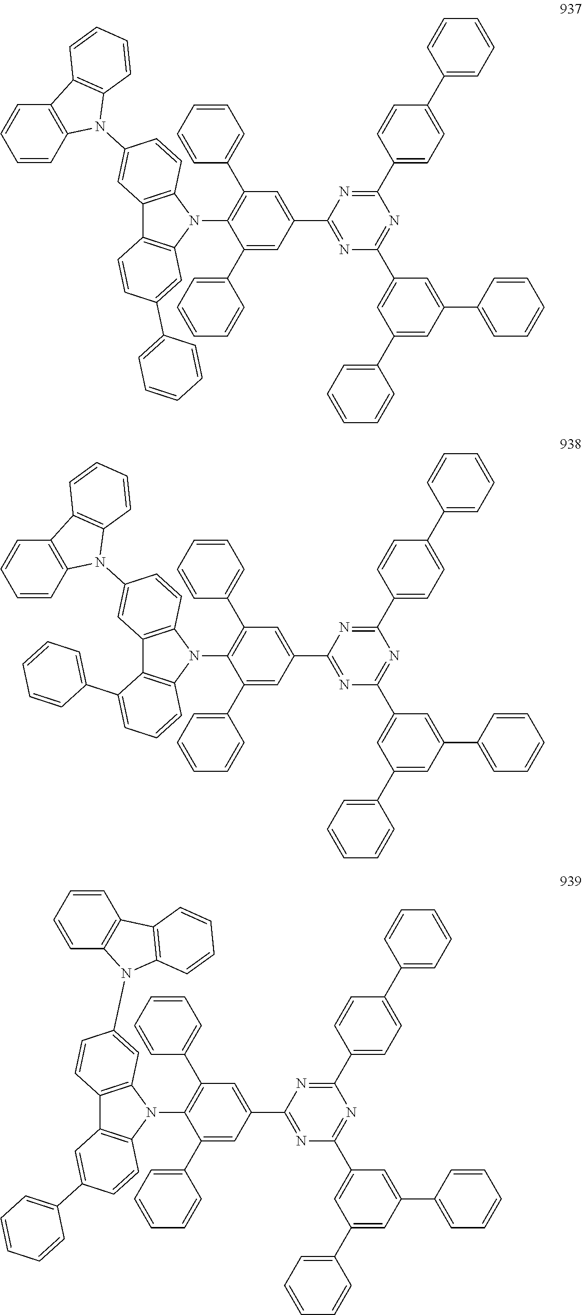 [SMILES: c1ccc(-c2ccc(-c3nc(-c4cc(-c5ccccc5)cc(-c5ccccc5)c4)nc(-c4cc(-c5ccccc5)c(-n5c6ccc(-n7c8ccccc8c8ccccc87)cc6c6ccc(-c7ccccc7)cc65)c(-c5ccccc5)c4)n3)cc2)cc1 ; c1ccc(-c2ccc(-c3nc(-c4cc(-c5ccccc5)cc(-c5ccccc5)c4)nc(-c4cc(-c5ccccc5)c(-n5c6ccc(-n7c8ccccc8c8ccccc87)cc6c6c(-c7ccccc7)cccc65)c(-c5ccccc5)c4)n3)cc2)cc1 ; c1ccc(-c2ccc(-c3nc(-c4cc(-c5ccccc5)cc(-c5ccccc5)c4)nc(-c4cc(-c5ccccc5)c(-n5c6ccc(-c7ccccc7)cc6c6ccc(-n7c8ccccc8c8ccccc87)cc65)c(-c5ccccc5)c4)n3)cc2)cc1]

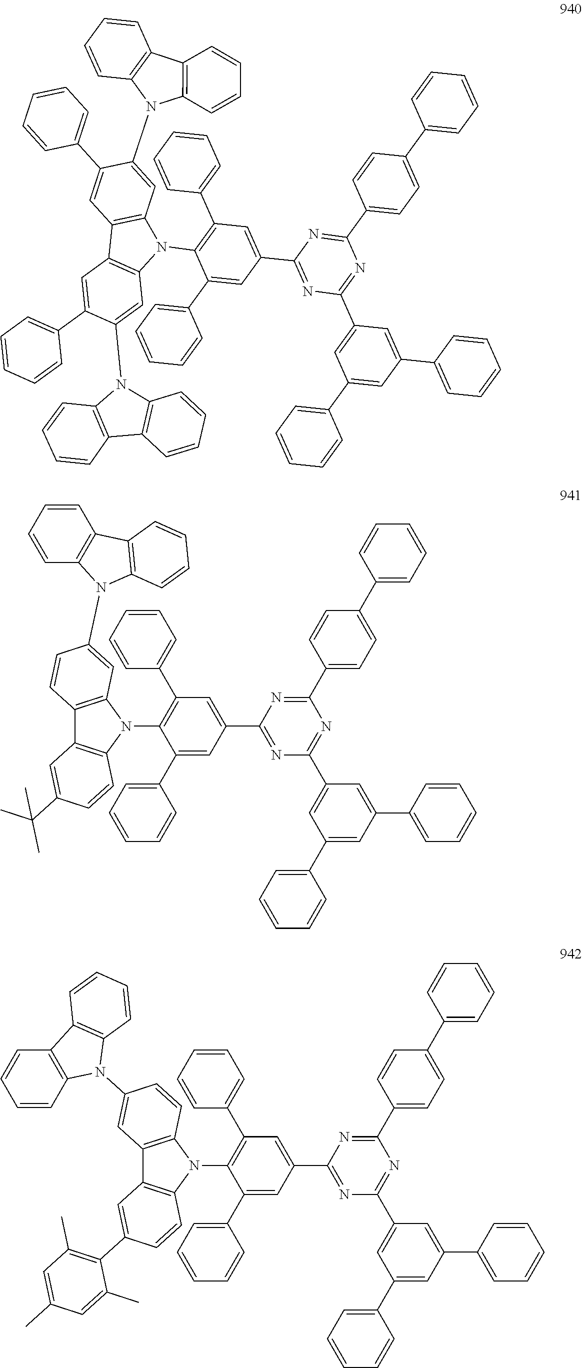

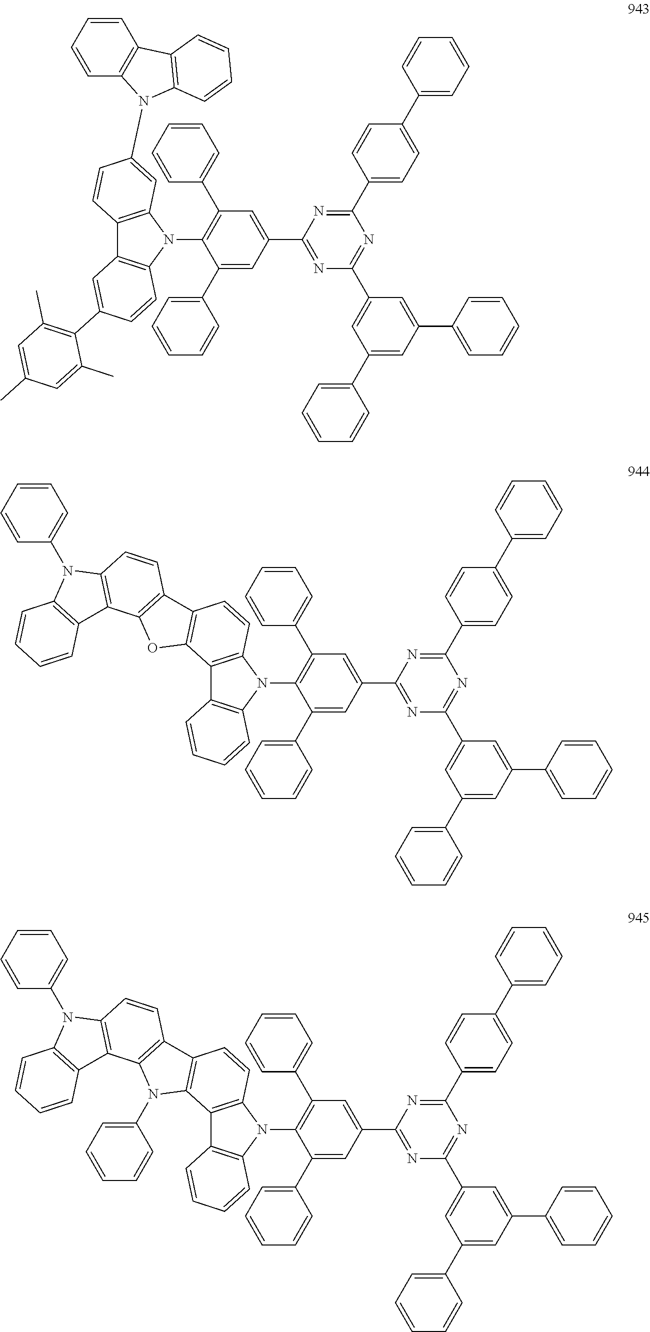 [SMILES: Cc1cc(C)c(-c2ccc3c(c2)c2ccc(-n4c5ccccc5c5ccccc54)cc2n3-c2c(-c3ccccc3)cc(-c3nc(-c4ccc(-c5ccccc5)cc4)nc(-c4cc(-c5ccccc5)cc(-c5ccccc5)c4)n3)cc2-c2ccccc2)c(C)c1 ; c1ccc(-c2ccc(-c3nc(-c4cc(-c5ccccc5)cc(-c5ccccc5)c4)nc(-c4cc(-c5ccccc5)c(-n5c6ccccc6c6c7oc8c(ccc9c8c8ccccc8n9-c8ccccc8)c7ccc65)c(-c5ccccc5)c4)n3)cc2)cc1 ; c1ccc(-c2ccc(-c3nc(-c4cc(-c5ccccc5)cc(-c5ccccc5)c4)nc(-c4cc(-c5ccccc5)c(-n5c6ccccc6c6c5ccc5c7ccc8c(c9ccccc9n8-c8ccccc8)c7n(-c7ccccc7)c56)c(-c5ccccc5)c4)n3)cc2)cc1]

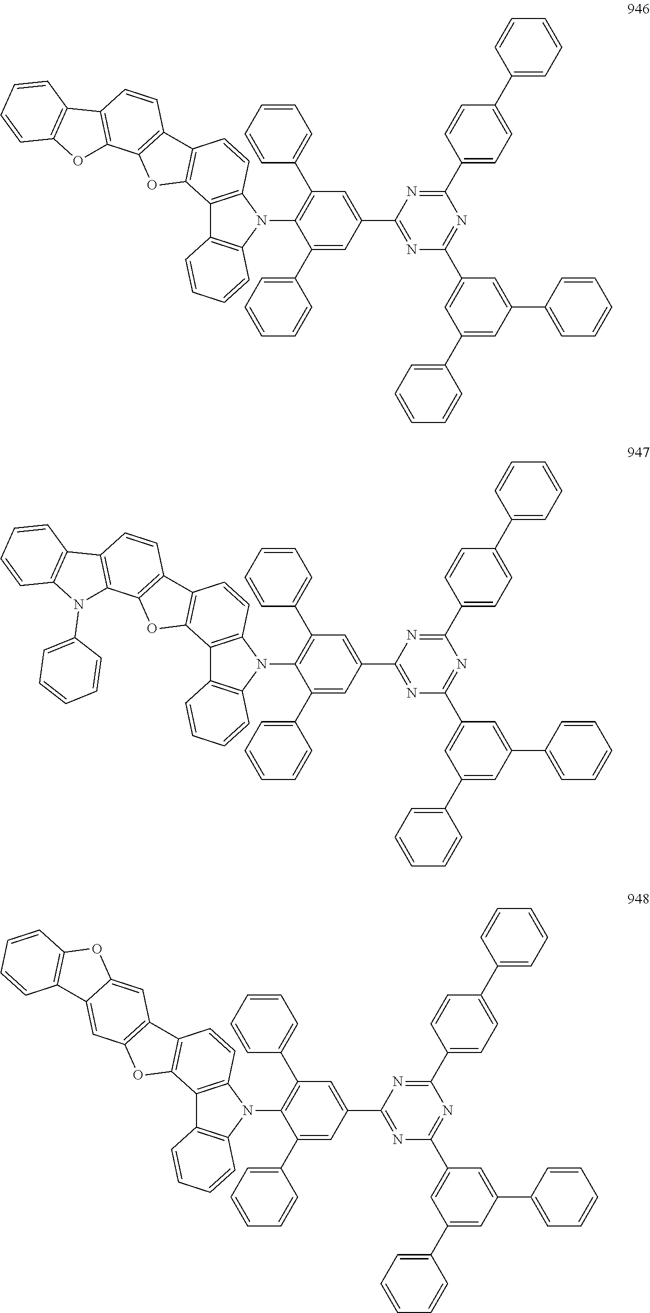 [SMILES: c1ccc(-c2ccc(-c3nc(-c4cc(-c5ccccc5)cc(-c5ccccc5)c4)nc(-c4cc(-c5ccccc5)c(-n5c6ccccc6c6c7oc8c(ccc9c%10ccccc%10oc98)c7ccc65)c(-c5ccccc5)c4)n3)cc2)cc1 ; c1ccc(-c2ccc(-c3nc(-c4cc(-c5ccccc5)cc(-c5ccccc5)c4)nc(-c4cc(-c5ccccc5)c(-n5c6ccccc6c6c7oc8c(ccc9c%10ccccc%10n(-c%10ccccc%10)c98)c7ccc65)c(-c5ccccc5)c4)n3)cc2)cc1 ; c1ccc(-c2ccc(-c3nc(-c4cc(-c5ccccc5)cc(-c5ccccc5)c4)nc(-c4cc(-c5ccccc5)c(-n5c6ccccc6c6c7oc8cc9c(cc8c7ccc65)oc5ccccc59)c(-c5ccccc5)c4)n3)cc2)cc1]

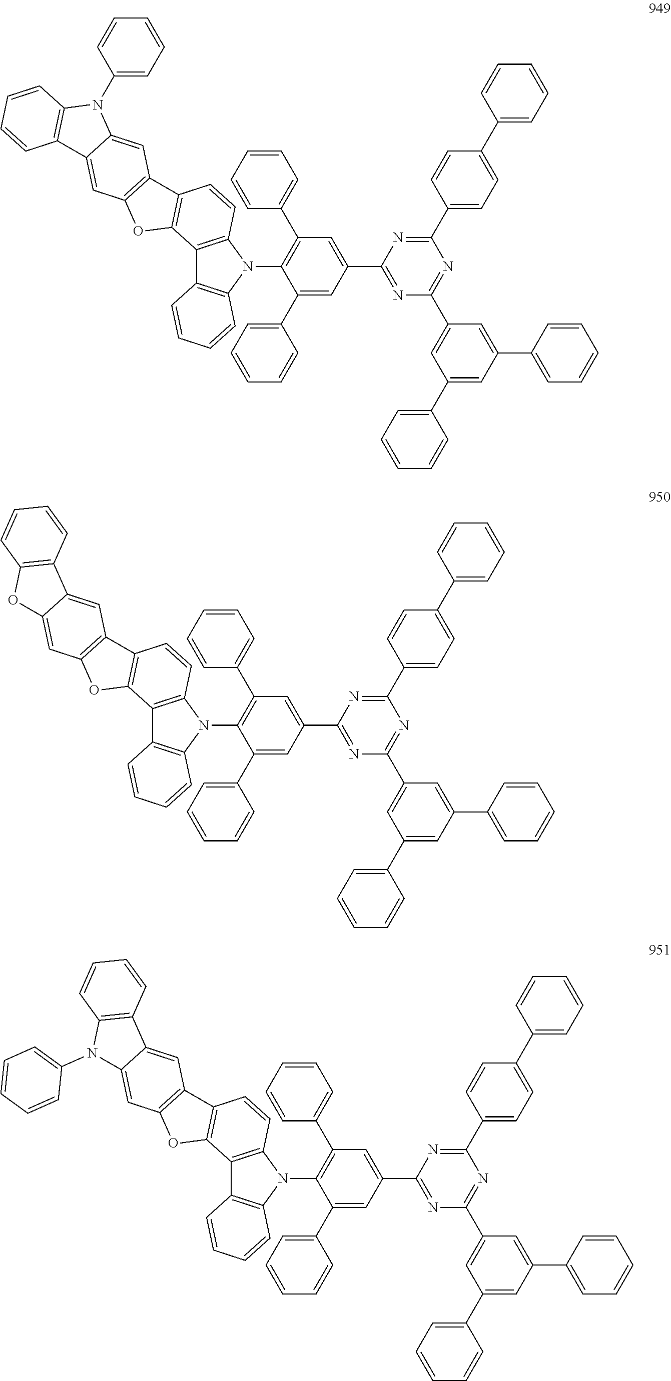 [SMILES: c1ccc(-c2ccc(-c3nc(-c4cc(-c5ccccc5)cc(-c5ccccc5)c4)nc(-c4cc(-c5ccccc5)c(-n5c6ccccc6c6c7oc8cc9c%10ccccc%10n(-c%10ccccc%10)c9cc8c7ccc65)c(-c5ccccc5)c4)n3)cc2)cc1 ; c1ccc(-c2ccc(-c3nc(-c4cc(-c5ccccc5)cc(-c5ccccc5)c4)nc(-c4cc(-c5ccccc5)c(-n5c6ccccc6c6c7oc8cc9oc%10ccccc%10c9cc8c7ccc65)c(-c5ccccc5)c4)n3)cc2)cc1 ; c1ccc(-c2ccc(-c3nc(-c4cc(-c5ccccc5)cc(-c5ccccc5)c4)nc(-c4cc(-c5ccccc5)c(-n5c6ccccc6c6c7oc8cc9c(cc8c7ccc65)c5ccccc5n9-c5ccccc5)c(-c5ccccc5)c4)n3)cc2)cc1]

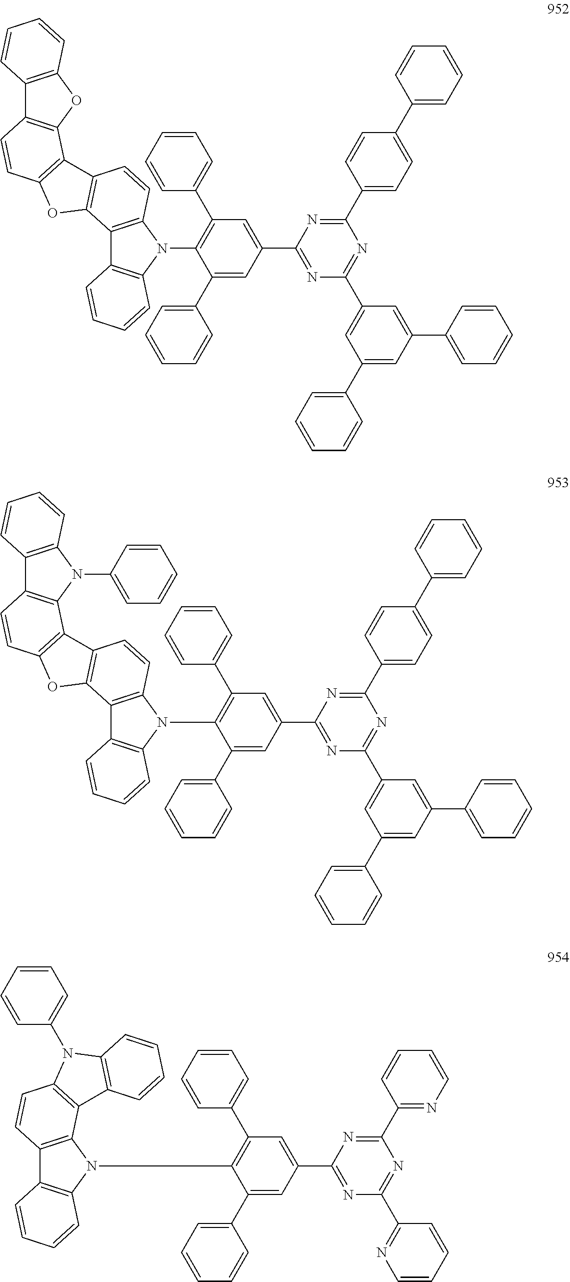 [SMILES: c1ccc(-c2ccc(-c3nc(-c4cc(-c5ccccc5)cc(-c5ccccc5)c4)nc(-c4cc(-c5ccccc5)c(-n5c6ccccc6c6c7oc8ccc9c%10ccccc%10oc9c8c7ccc65)c(-c5ccccc5)c4)n3)cc2)cc1 ; c1ccc(-c2ccc(-c3nc(-c4cc(-c5ccccc5)cc(-c5ccccc5)c4)nc(-c4cc(-c5ccccc5)c(-n5c6ccccc6c6c7oc8ccc9c%10ccccc%10n(-c%10ccccc%10)c9c8c7ccc65)c(-c5ccccc5)c4)n3)cc2)cc1 ; c1ccc(-c2cc(-c3nc(-c4ccccn4)nc(-c4ccccn4)n3)cc(-c3ccccc3)c2-n2c3ccccc3c3ccc4c(c5ccccc5n4-c4ccccc4)c32)cc1]

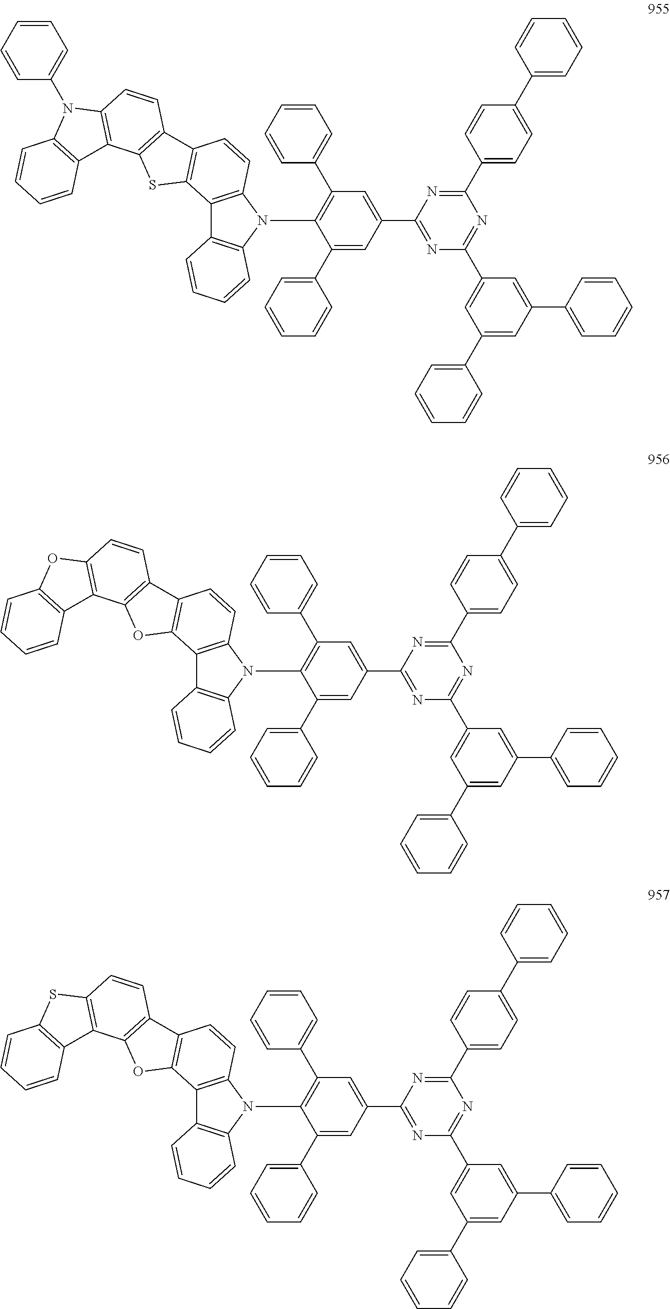 [SMILES: c1ccc(-c2ccc(-c3nc(-c4cc(-c5ccccc5)cc(-c5ccccc5)c4)nc(-c4cc(-c5ccccc5)c(-n5c6ccccc6c6c7sc8c(ccc9c8c8ccccc8n9-c8ccccc8)c7ccc65)c(-c5ccccc5)c4)n3)cc2)cc1 ; c1ccc(-c2ccc(-c3nc(-c4cc(-c5ccccc5)cc(-c5ccccc5)c4)nc(-c4cc(-c5ccccc5)c(-n5c6ccccc6c6c7oc8c(ccc9oc%10ccccc%10c98)c7ccc65)c(-c5ccccc5)c4)n3)cc2)cc1 ; c1ccc(-c2ccc(-c3nc(-c4cc(-c5ccccc5)cc(-c5ccccc5)c4)nc(-c4cc(-c5ccccc5)c(-n5c6ccccc6c6c7oc8c(ccc9sc%10ccccc%10c98)c7ccc65)c(-c5ccccc5)c4)n3)cc2)cc1]

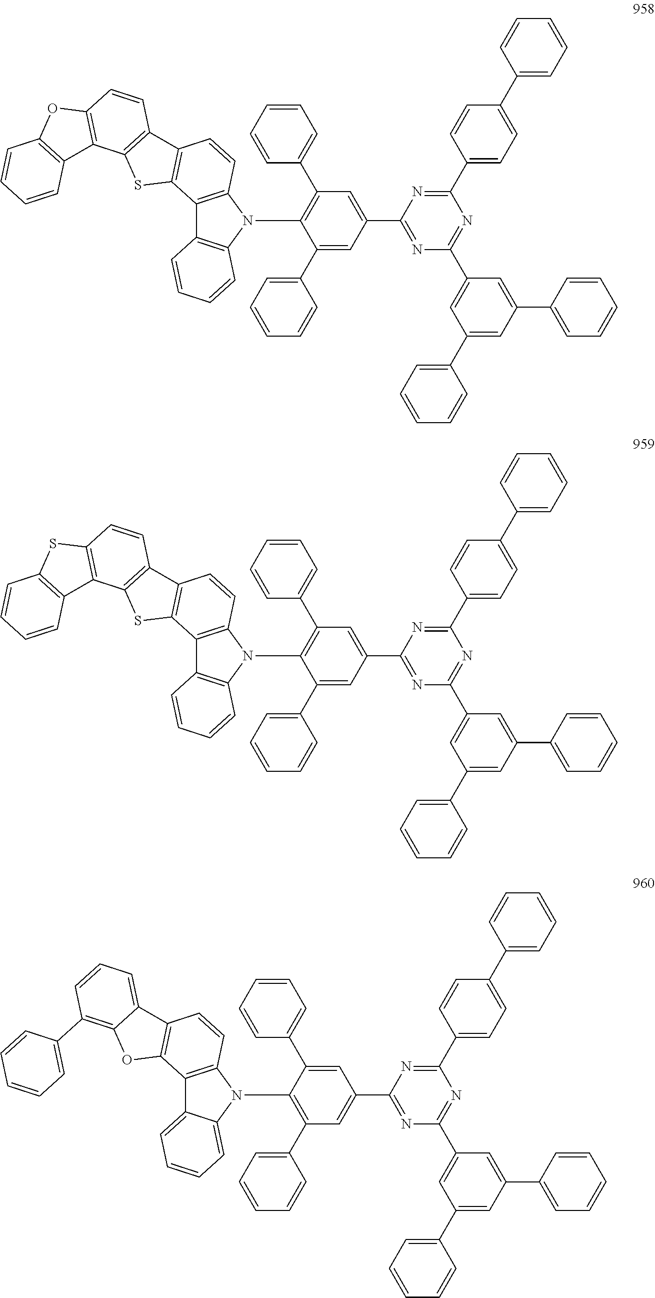 [SMILES: c1ccc(-c2ccc(-c3nc(-c4cc(-c5ccccc5)cc(-c5ccccc5)c4)nc(-c4cc(-c5ccccc5)c(-n5c6ccccc6c6c7sc8c(ccc9oc%10ccccc%10c98)c7ccc65)c(-c5ccccc5)c4)n3)cc2)cc1 ; c1ccc(-c2ccc(-c3nc(-c4cc(-c5ccccc5)cc(-c5ccccc5)c4)nc(-c4cc(-c5ccccc5)c(-n5c6ccccc6c6c7sc8c(ccc9sc%10ccccc%10c98)c7ccc65)c(-c5ccccc5)c4)n3)cc2)cc1 ; c1ccc(-c2ccc(-c3nc(-c4cc(-c5ccccc5)cc(-c5ccccc5)c4)nc(-c4cc(-c5ccccc5)c(-n5c6ccccc6c6c7oc8c(-c9ccccc9)cccc8c7ccc65)c(-c5ccccc5)c4)n3)cc2)cc1]

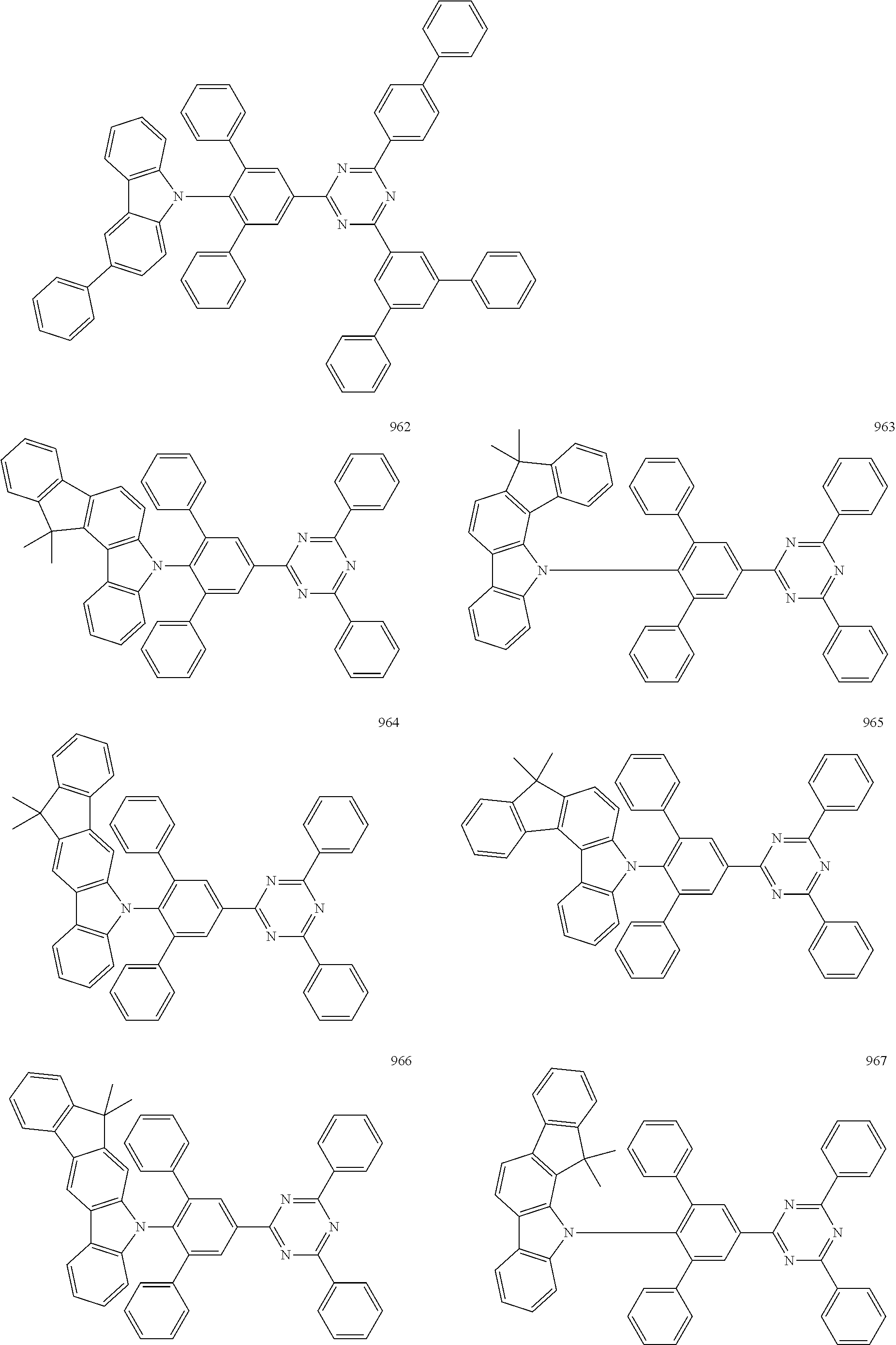

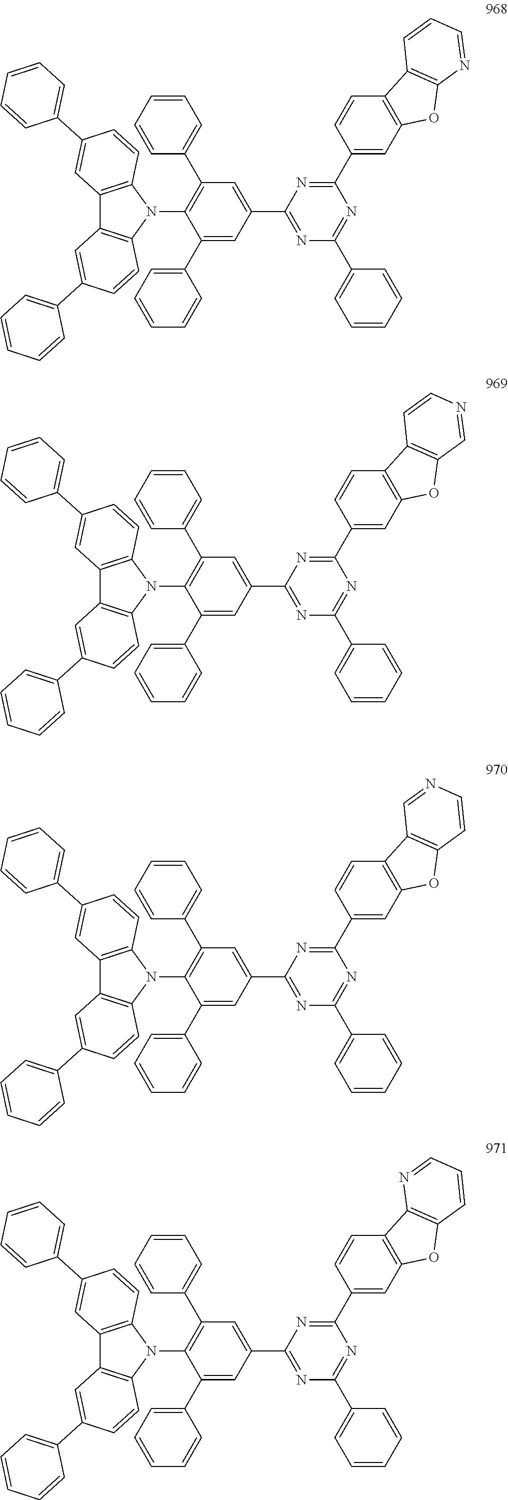 [SMILES: c1ccc(-c2ccc3c(c2)c2cc(-c4ccccc4)ccc2n3-c2c(-c3ccccc3)cc(-c3nc(-c4ccccc4)nc(-c4ccc5c(c4)oc4ncccc45)n3)cc2-c2ccccc2)cc1 ; c1ccc(-c2ccc3c(c2)c2cc(-c4ccccc4)ccc2n3-c2c(-c3ccccc3)cc(-c3nc(-c4ccccc4)nc(-c4ccc5c(c4)oc4cnccc45)n3)cc2-c2ccccc2)cc1 ; c1ccc(-c2ccc3c(c2)c2cc(-c4ccccc4)ccc2n3-c2c(-c3ccccc3)cc(-c3nc(-c4ccccc4)nc(-c4ccc5c(c4)oc4ccncc45)n3)cc2-c2ccccc2)cc1 ; c1ccc(-c2ccc3c(c2)c2cc(-c4ccccc4)ccc2n3-c2c(-c3ccccc3)cc(-c3nc(-c4ccccc4)nc(-c4ccc5c(c4)oc4cccnc45)n3)cc2-c2ccccc2)cc1]

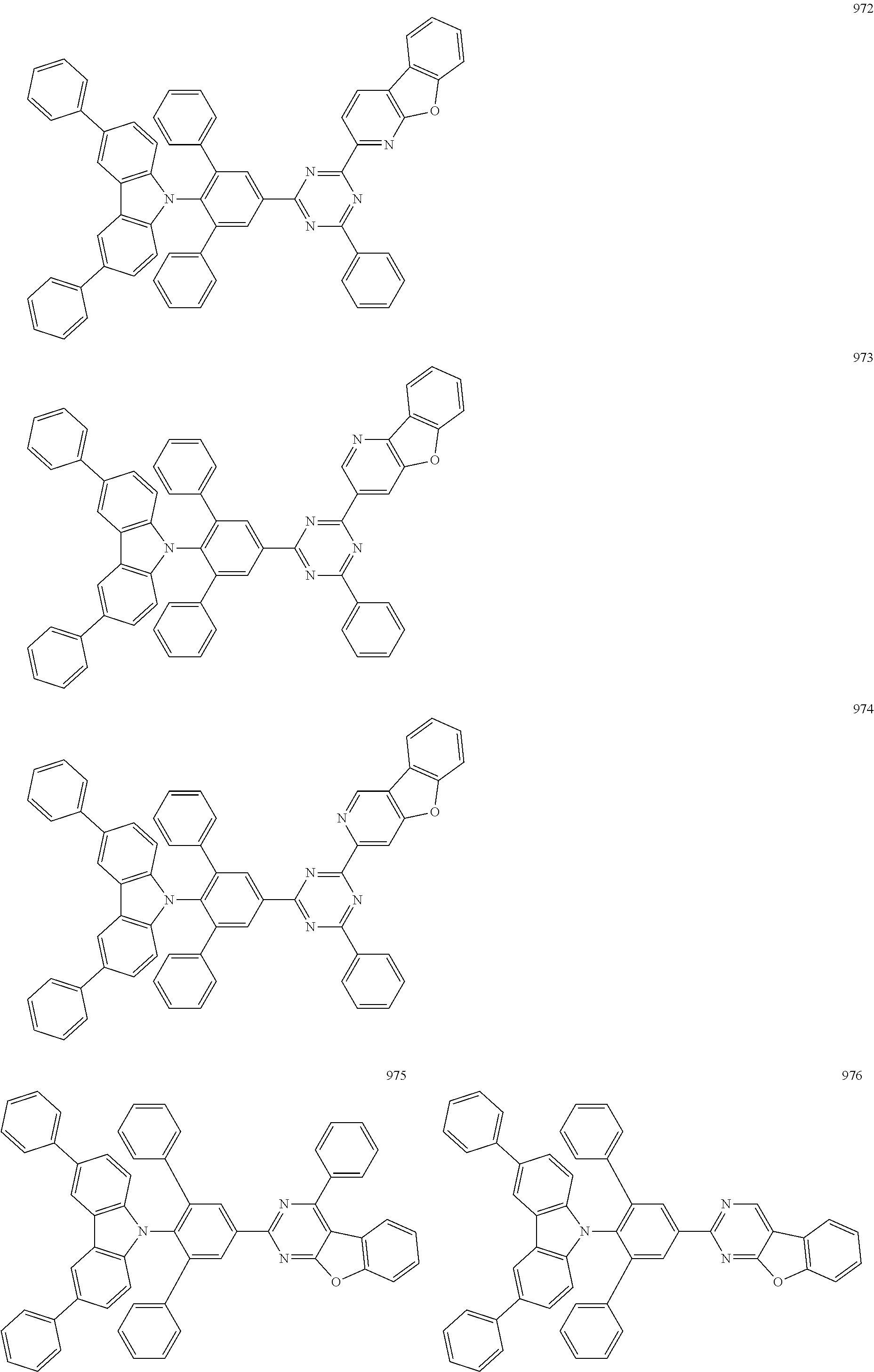

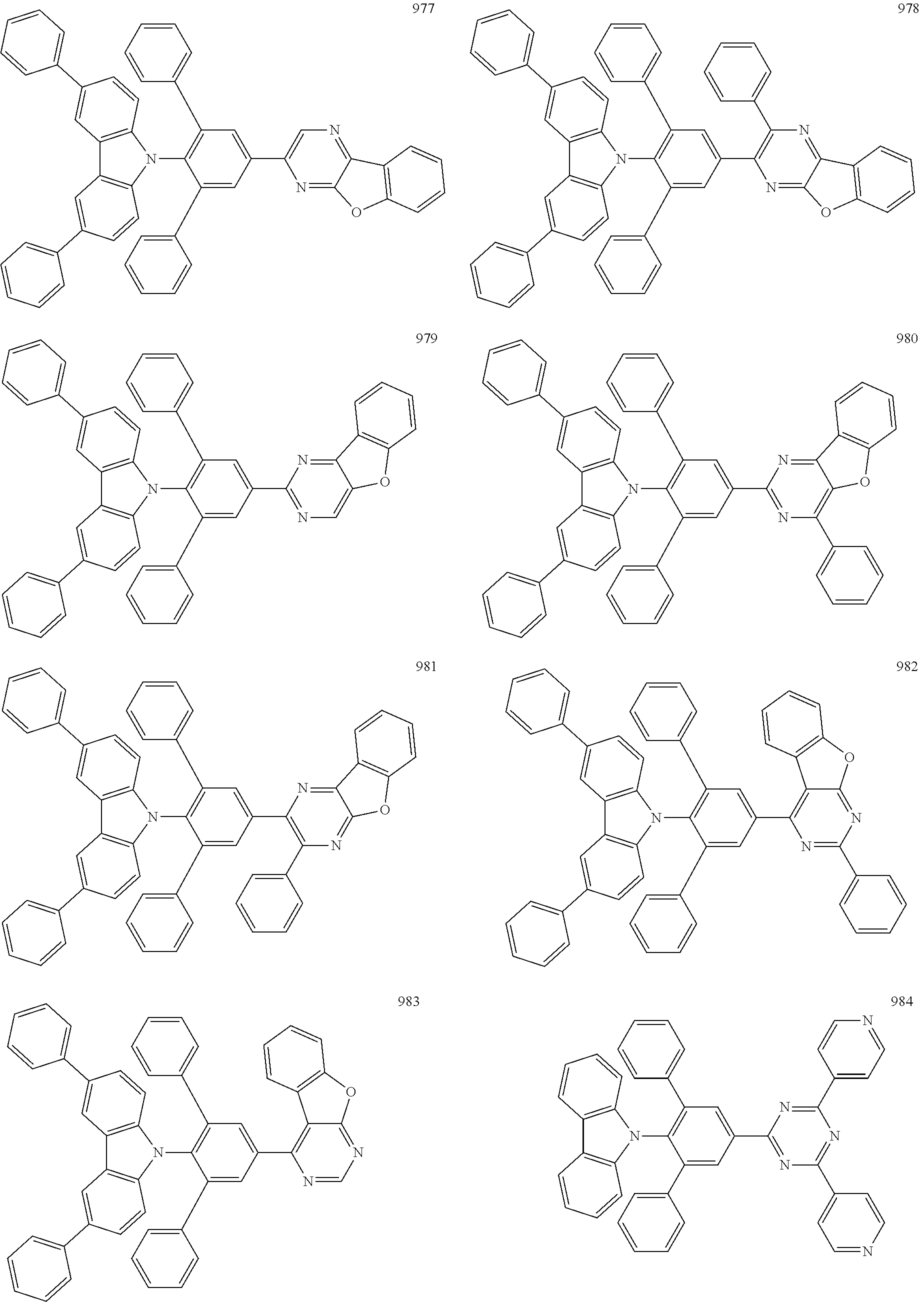

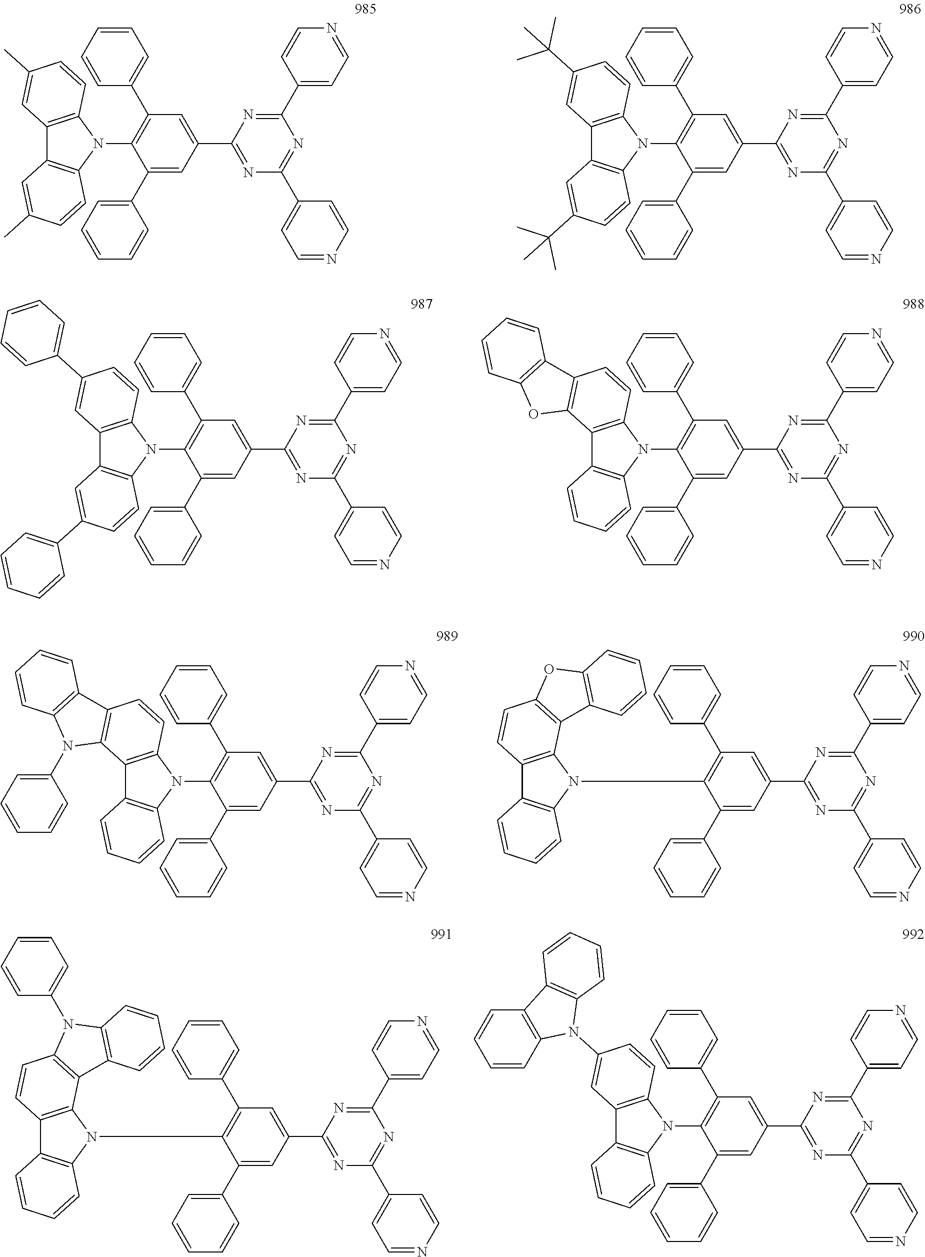

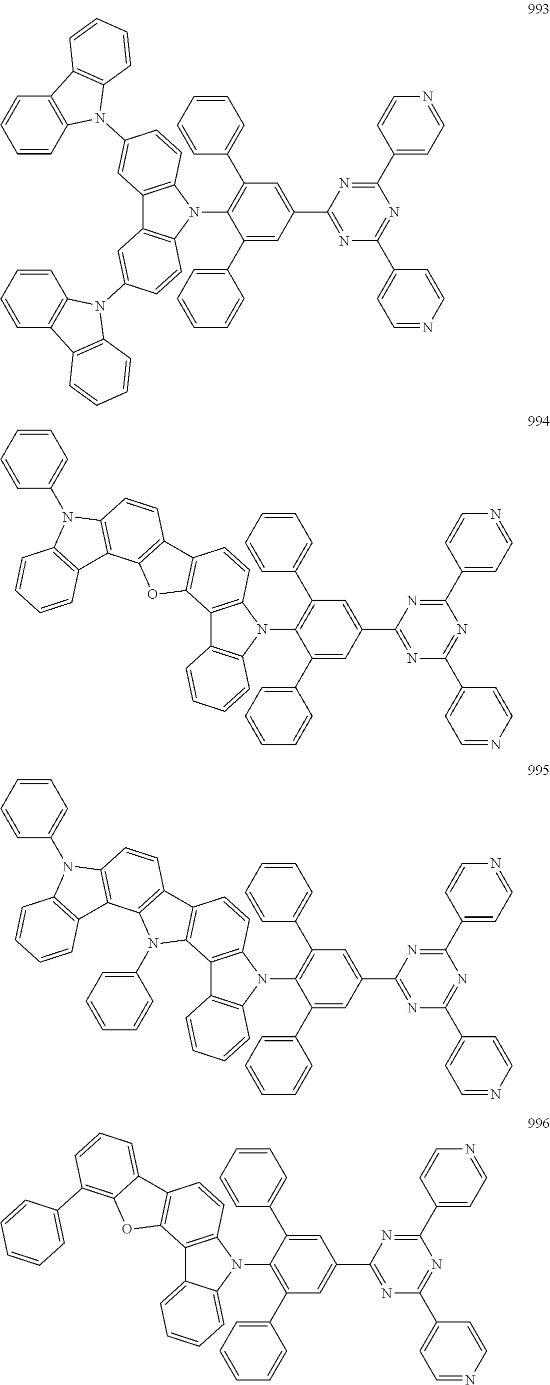 [SMILES: c1ccc(-c2cc(-c3nc(-c4ccncc4)nc(-c4ccncc4)n3)cc(-c3ccccc3)c2-n2c3ccc(-n4c5ccccc5c5ccccc54)cc3c3cc(-n4c5ccccc5c5ccccc54)ccc32)cc1 ; c1ccc(-c2cc(-c3nc(-c4ccncc4)nc(-c4ccncc4)n3)cc(-c3ccccc3)c2-n2c3ccccc3c3c4oc5c(ccc6c5c5ccccc5n6-c5ccccc5)c4ccc32)cc1 ; c1ccc(-c2cc(-c3nc(-c4ccncc4)nc(-c4ccncc4)n3)cc(-c3ccccc3)c2-n2c3ccccc3c3c2ccc2c4ccc5c(c6ccccc6n5-c5ccccc5)c4n(-c4ccccc4)c23)cc1 ; c1ccc(-c2cc(-c3nc(-c4ccncc4)nc(-c4ccncc4)n3)cc(-c3ccccc3)c2-n2c3ccccc3c3c4oc5c(-c6ccccc6)cccc5c4ccc32)cc1]

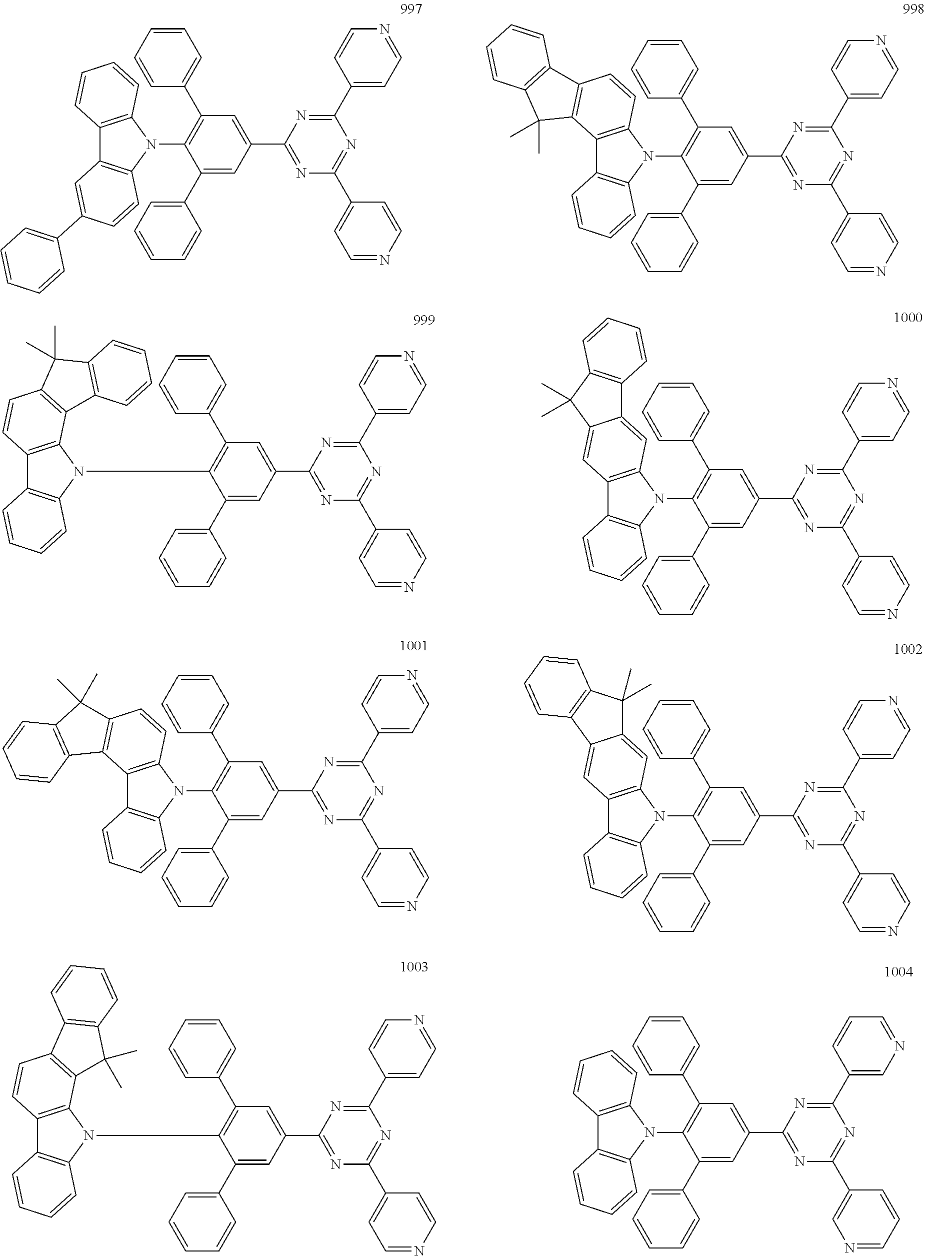

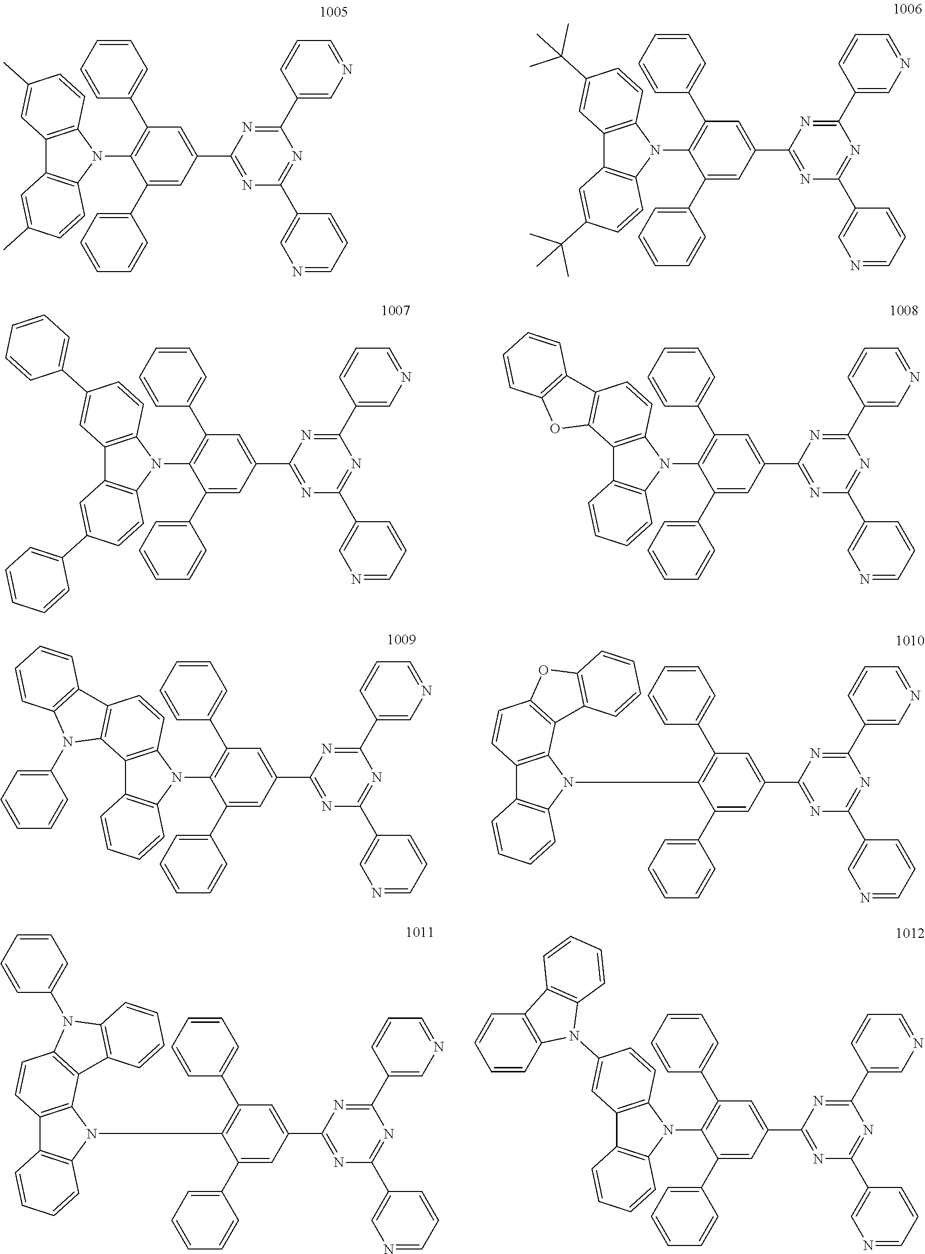

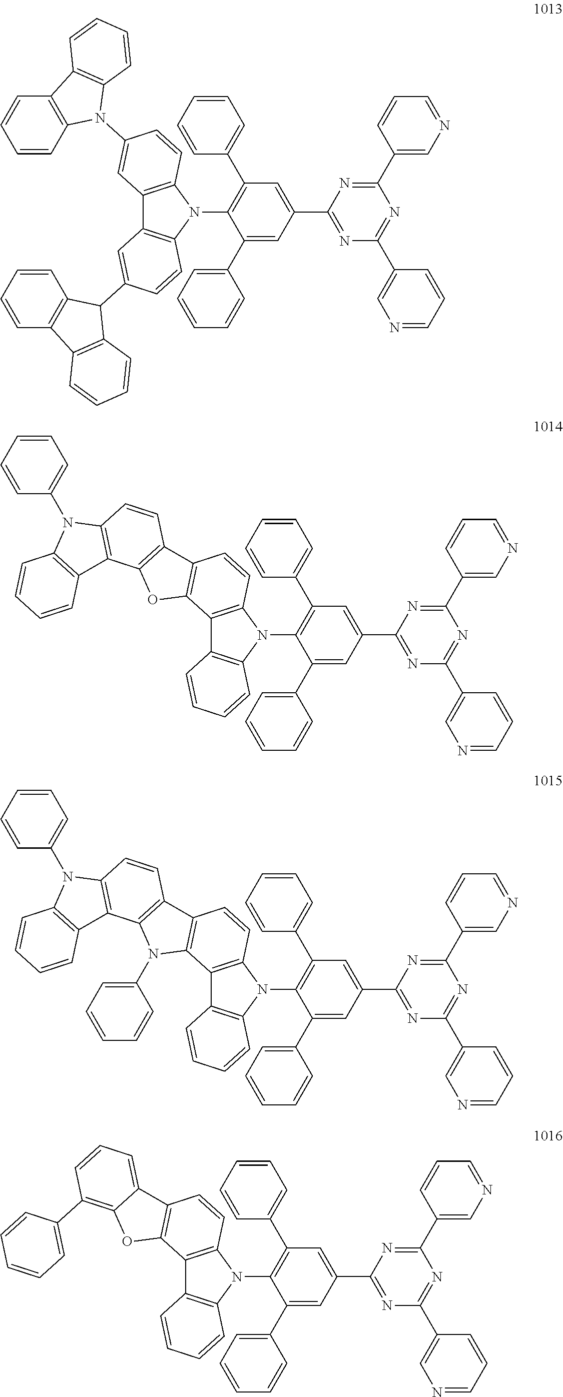 [SMILES: c1ccc(-c2cc(-c3nc(-c4cccnc4)nc(-c4cccnc4)n3)cc(-c3ccccc3)c2-n2c3ccc(C4c5ccccc5-c5ccccc54)cc3c3cc(-n4c5ccccc5c5ccccc54)ccc32)cc1 ; c1ccc(-c2cc(-c3nc(-c4cccnc4)nc(-c4cccnc4)n3)cc(-c3ccccc3)c2-n2c3ccccc3c3c4oc5c(ccc6c5c5ccccc5n6-c5ccccc5)c4ccc32)cc1 ; c1ccc(-c2cc(-c3nc(-c4cccnc4)nc(-c4cccnc4)n3)cc(-c3ccccc3)c2-n2c3ccccc3c3c2ccc2c4ccc5c(c6ccccc6n5-c5ccccc5)c4n(-c4ccccc4)c23)cc1 ; c1ccc(-c2cc(-c3nc(-c4cccnc4)nc(-c4cccnc4)n3)cc(-c3ccccc3)c2-n2c3ccccc3c3c4oc5c(-c6ccccc6)cccc5c4ccc32)cc1]

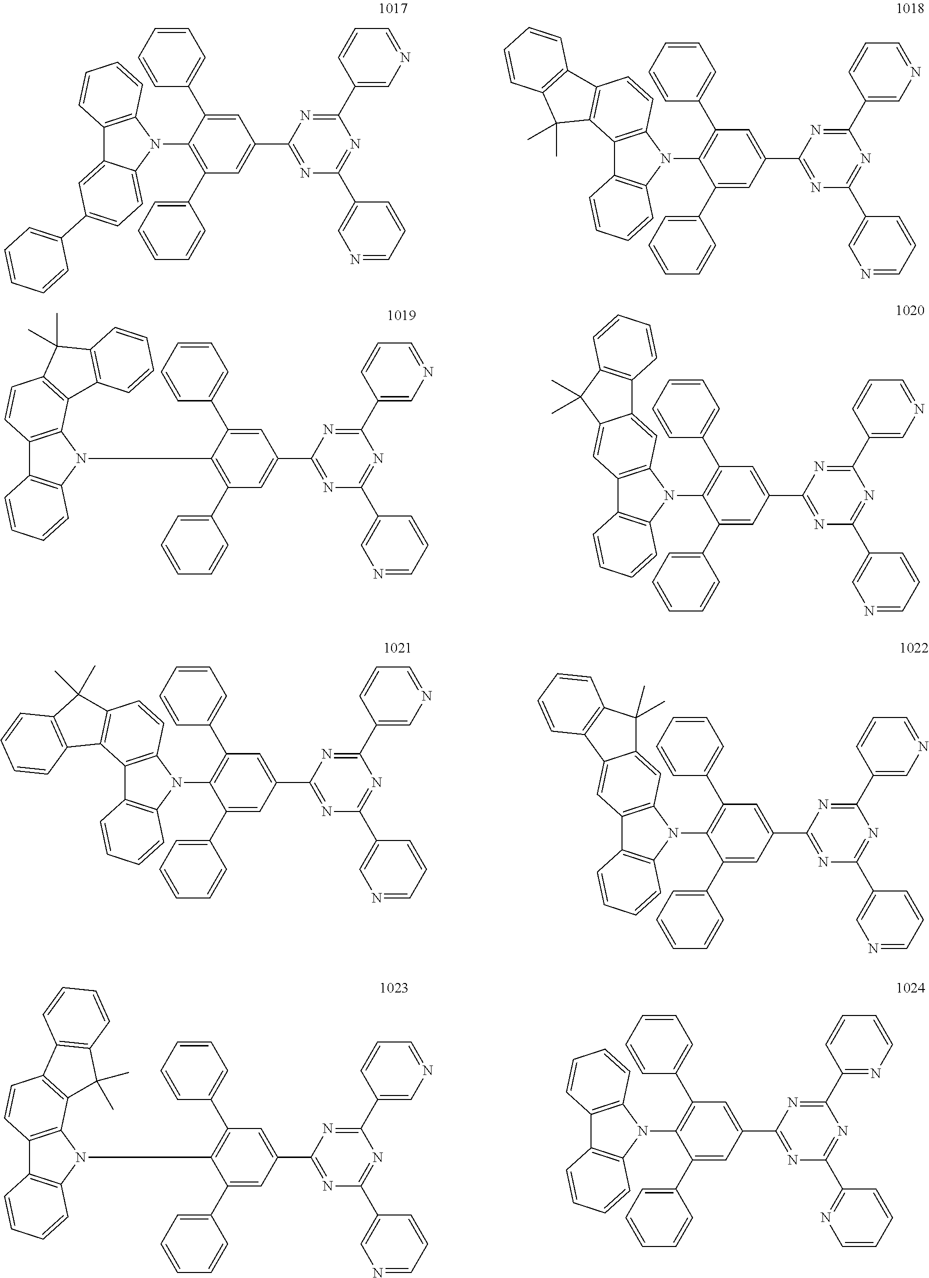

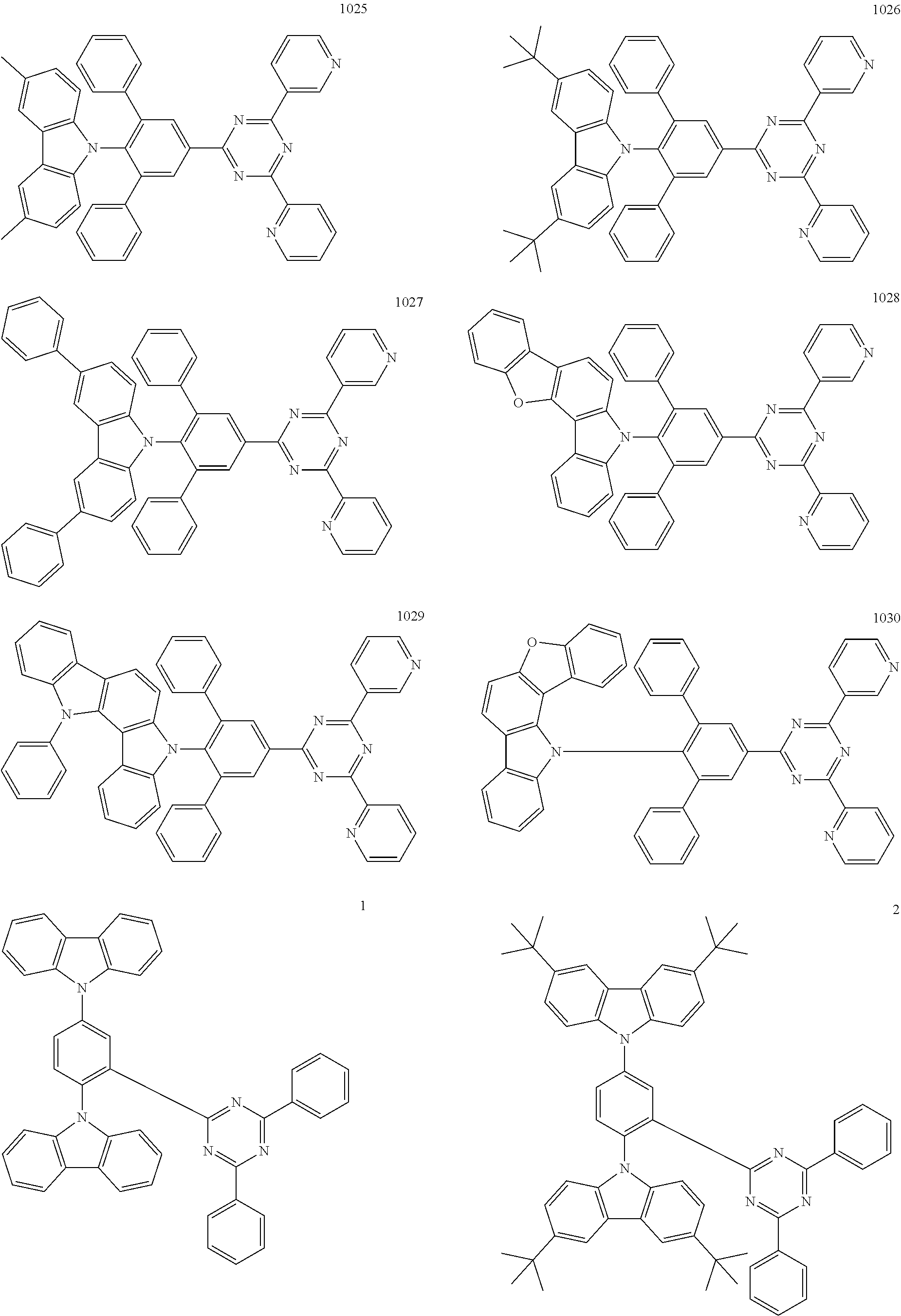

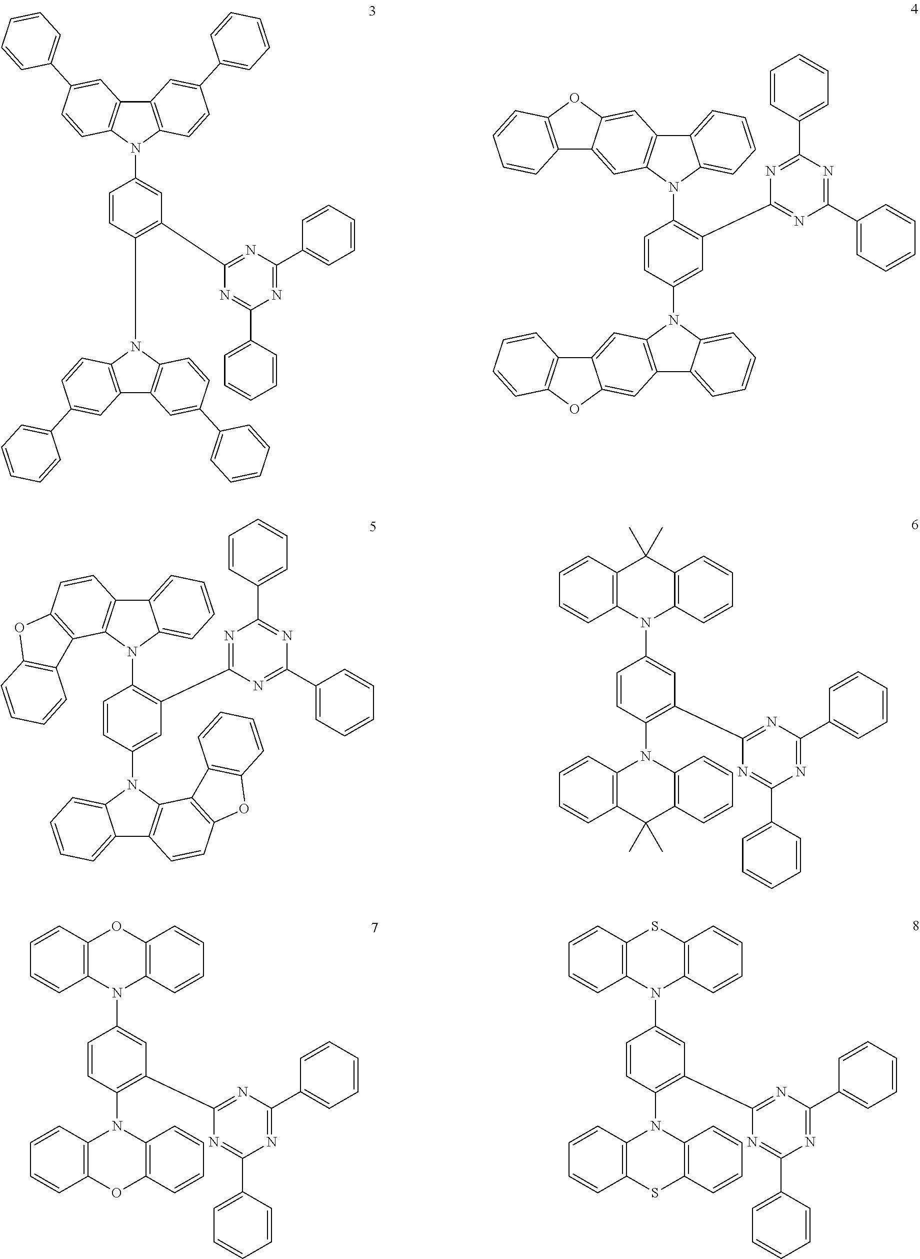

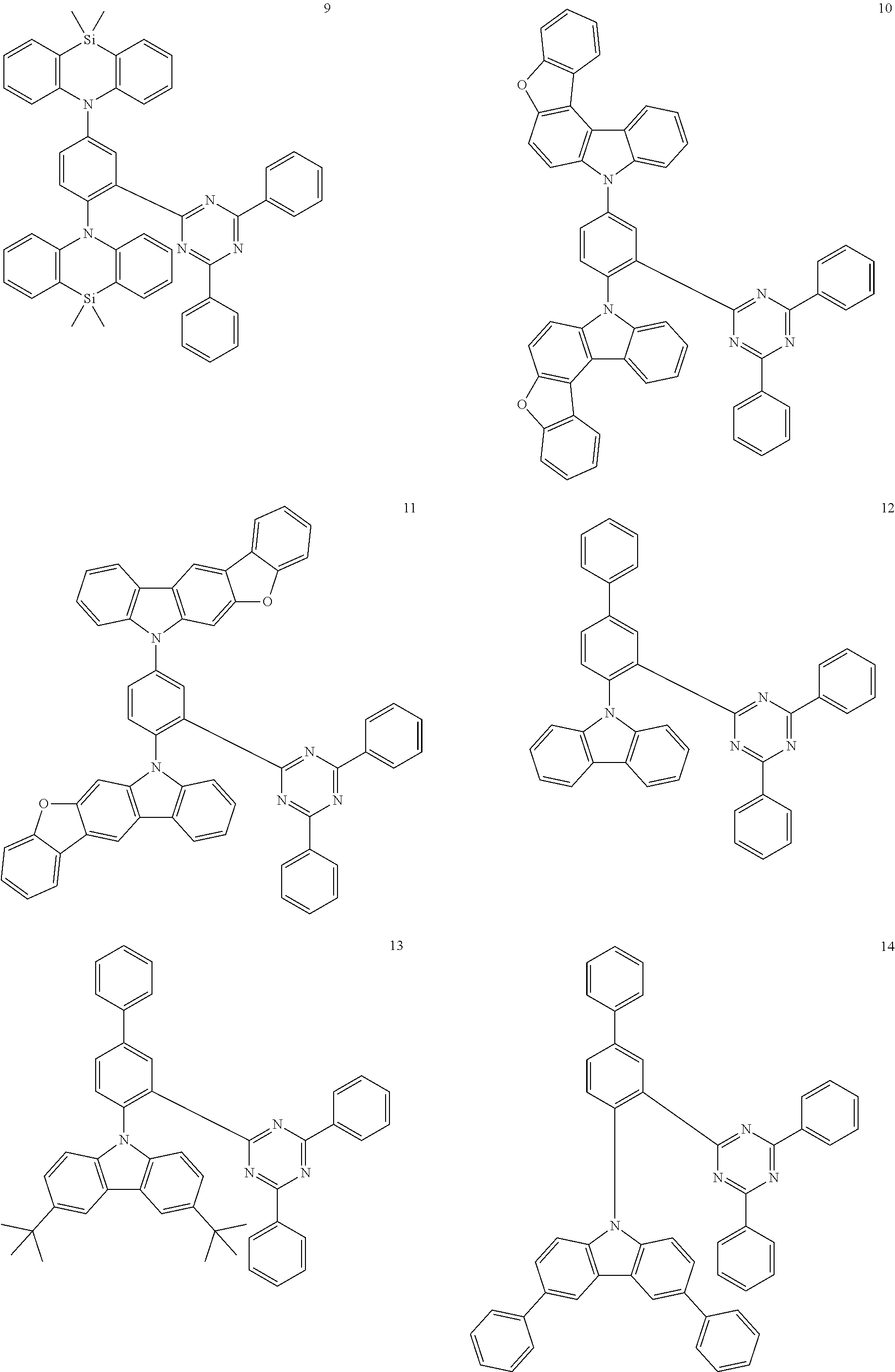

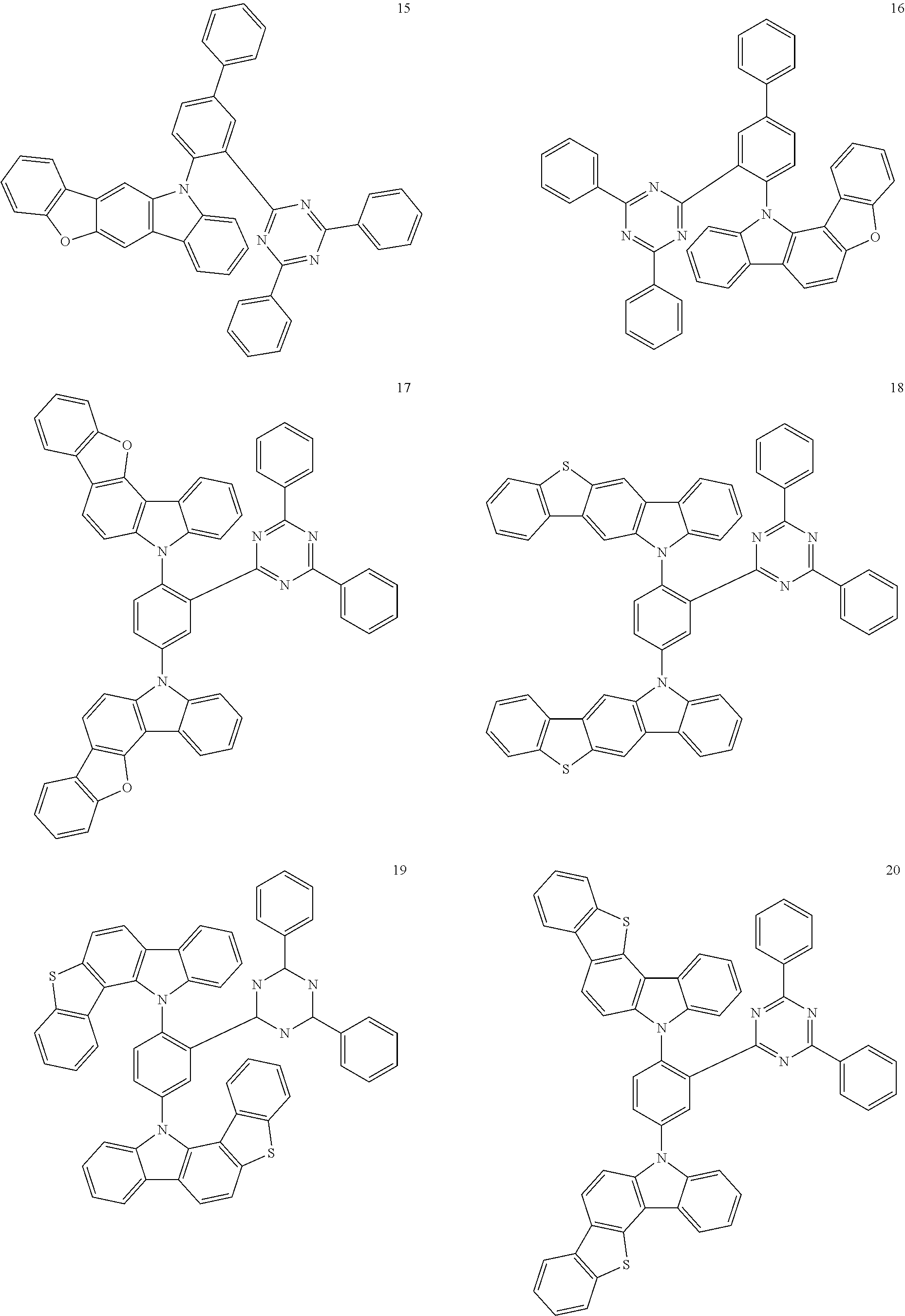

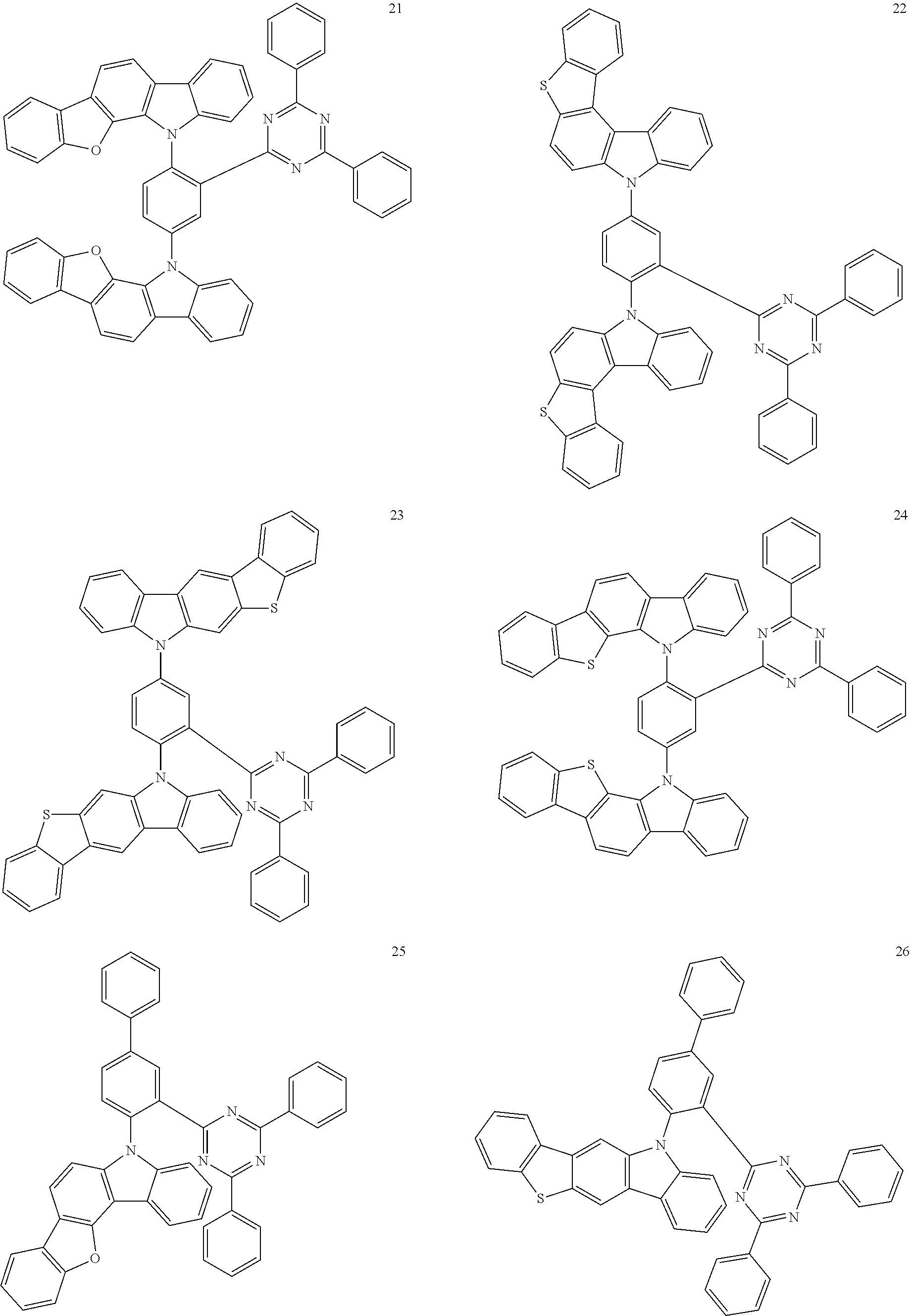

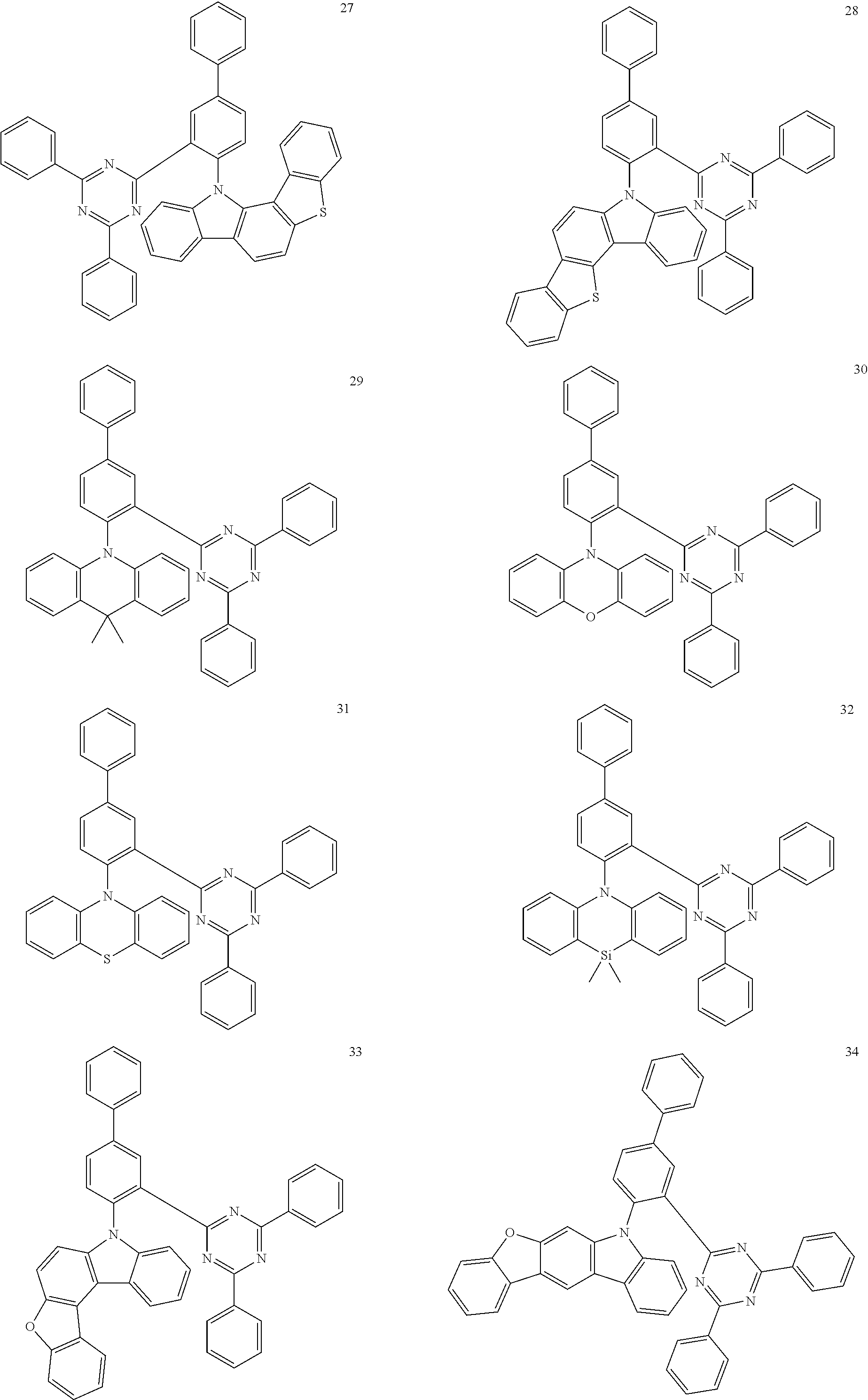

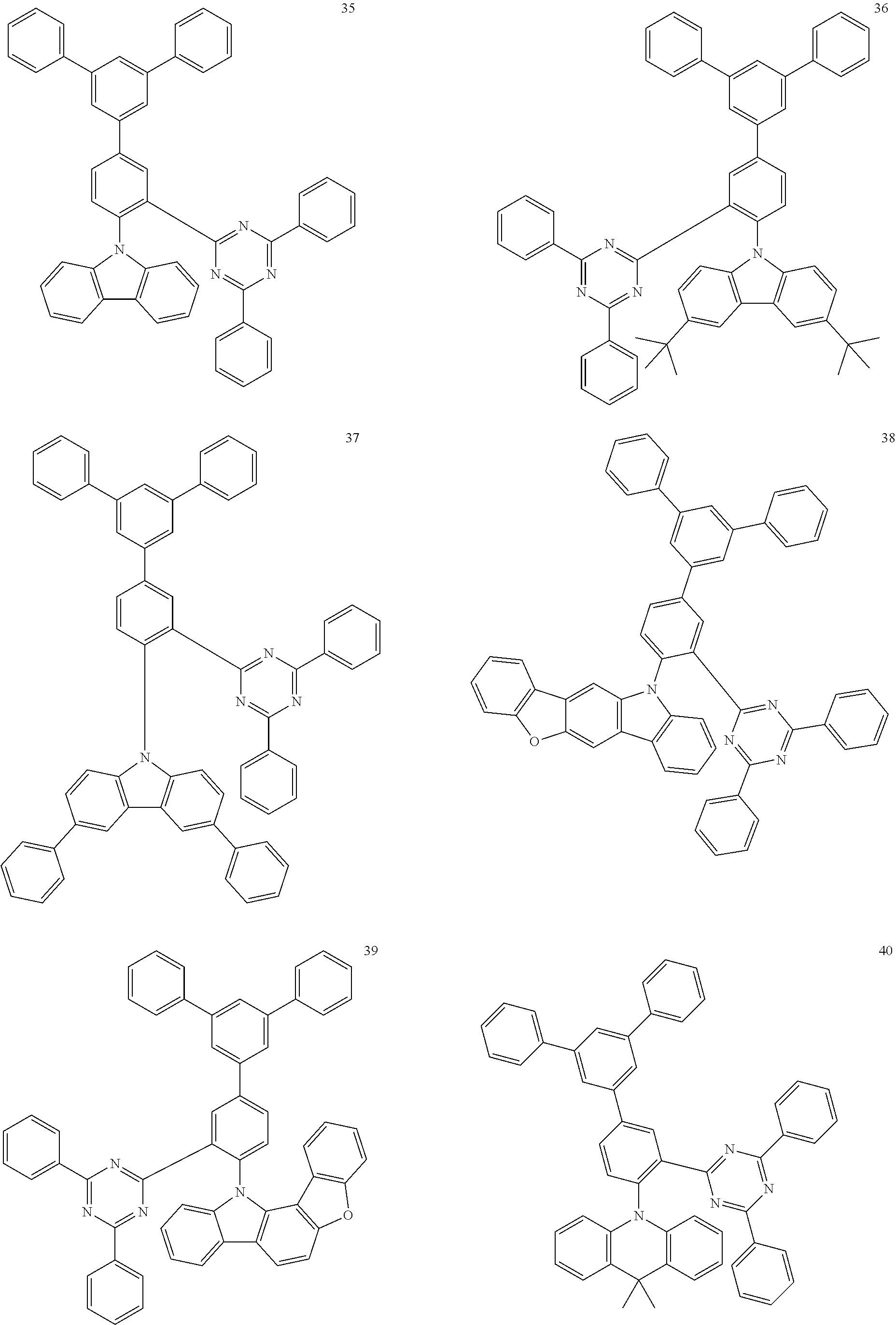

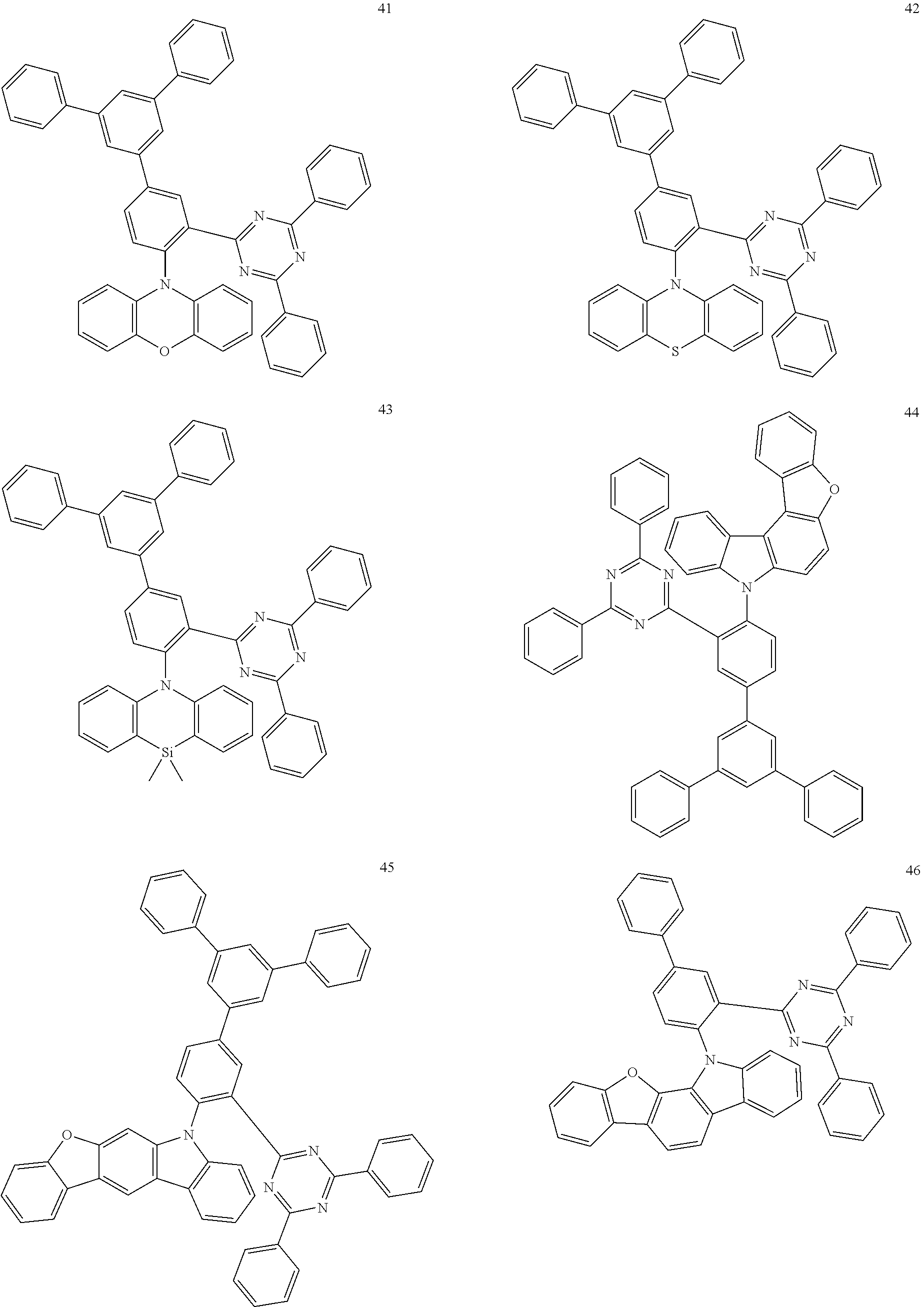

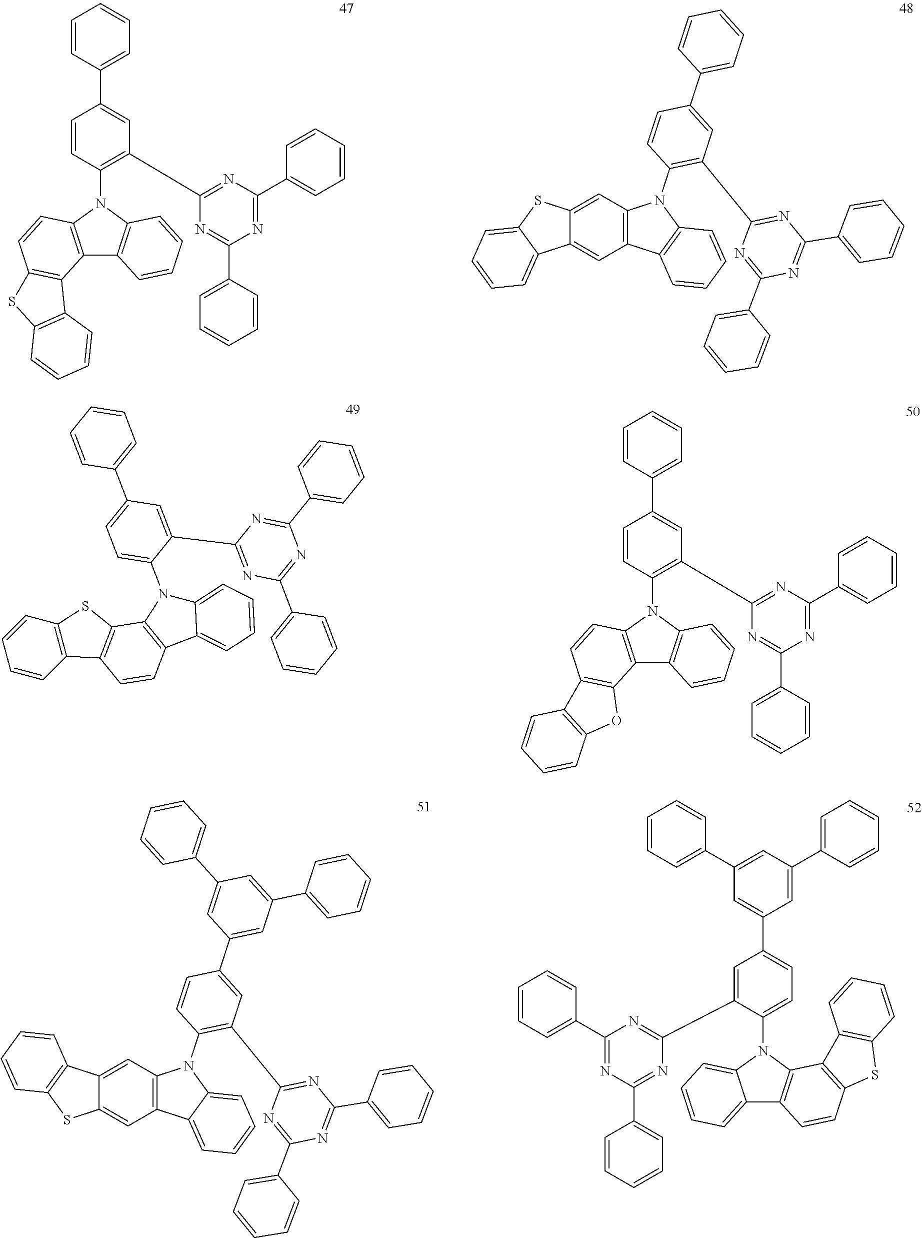

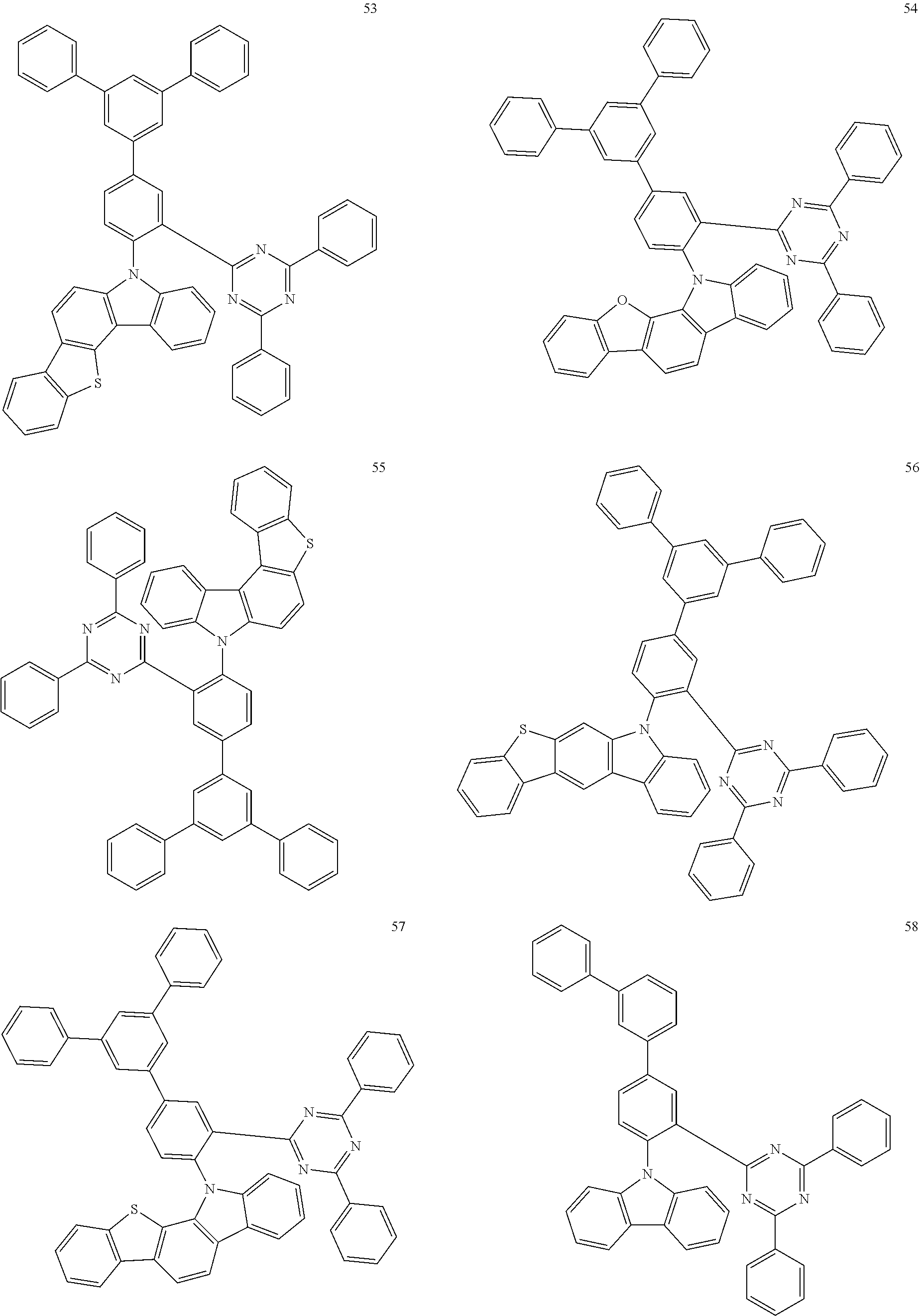

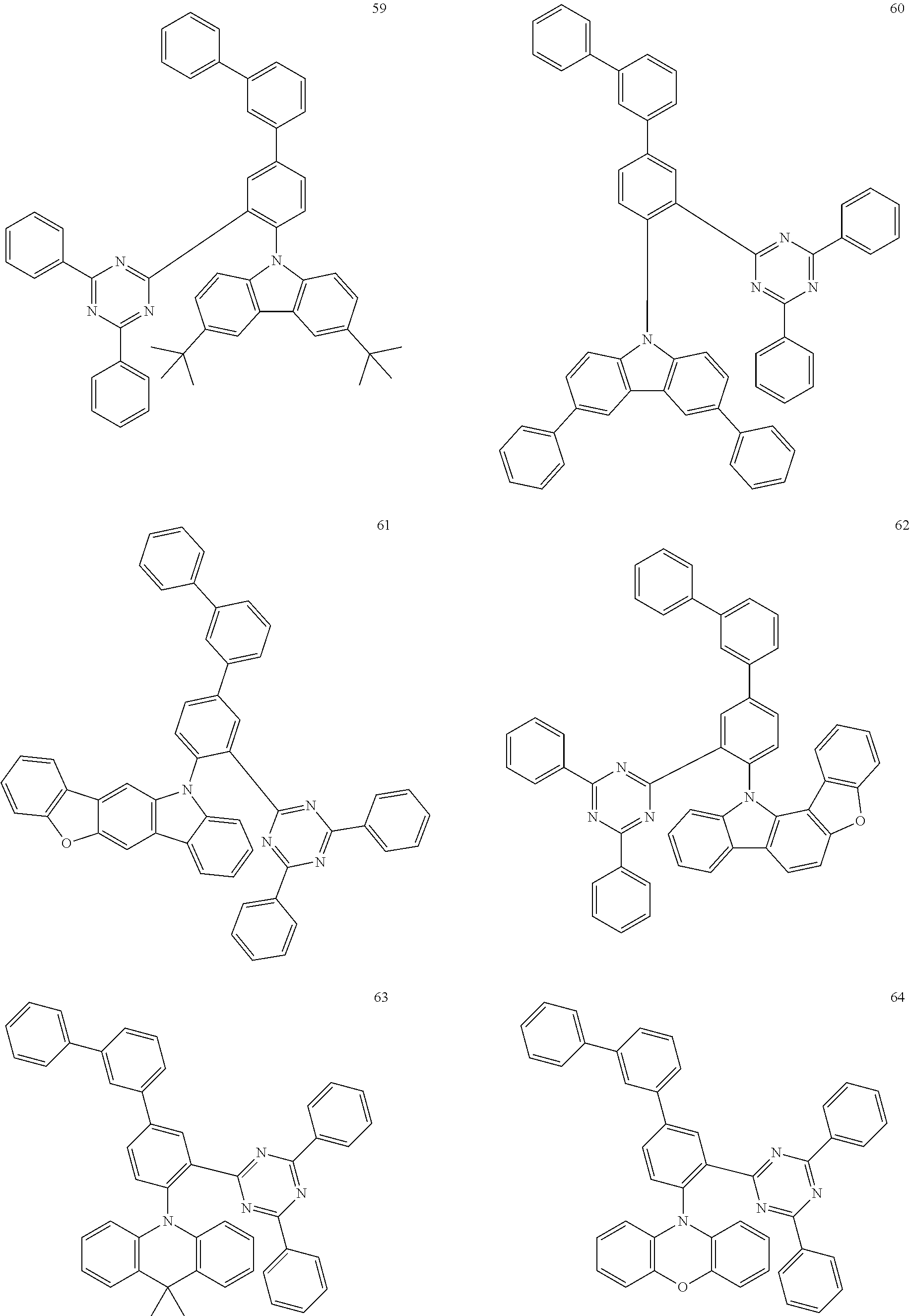

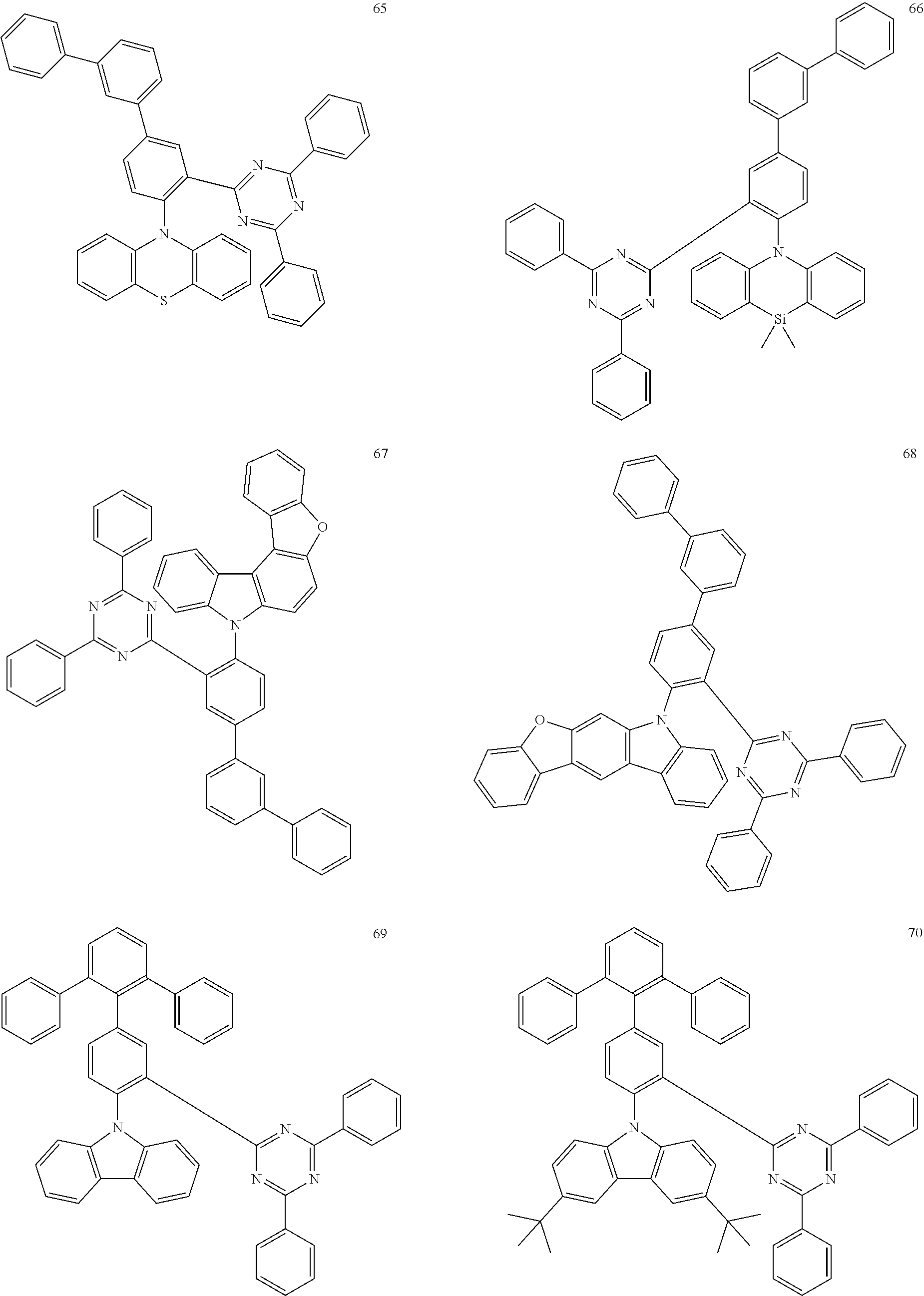

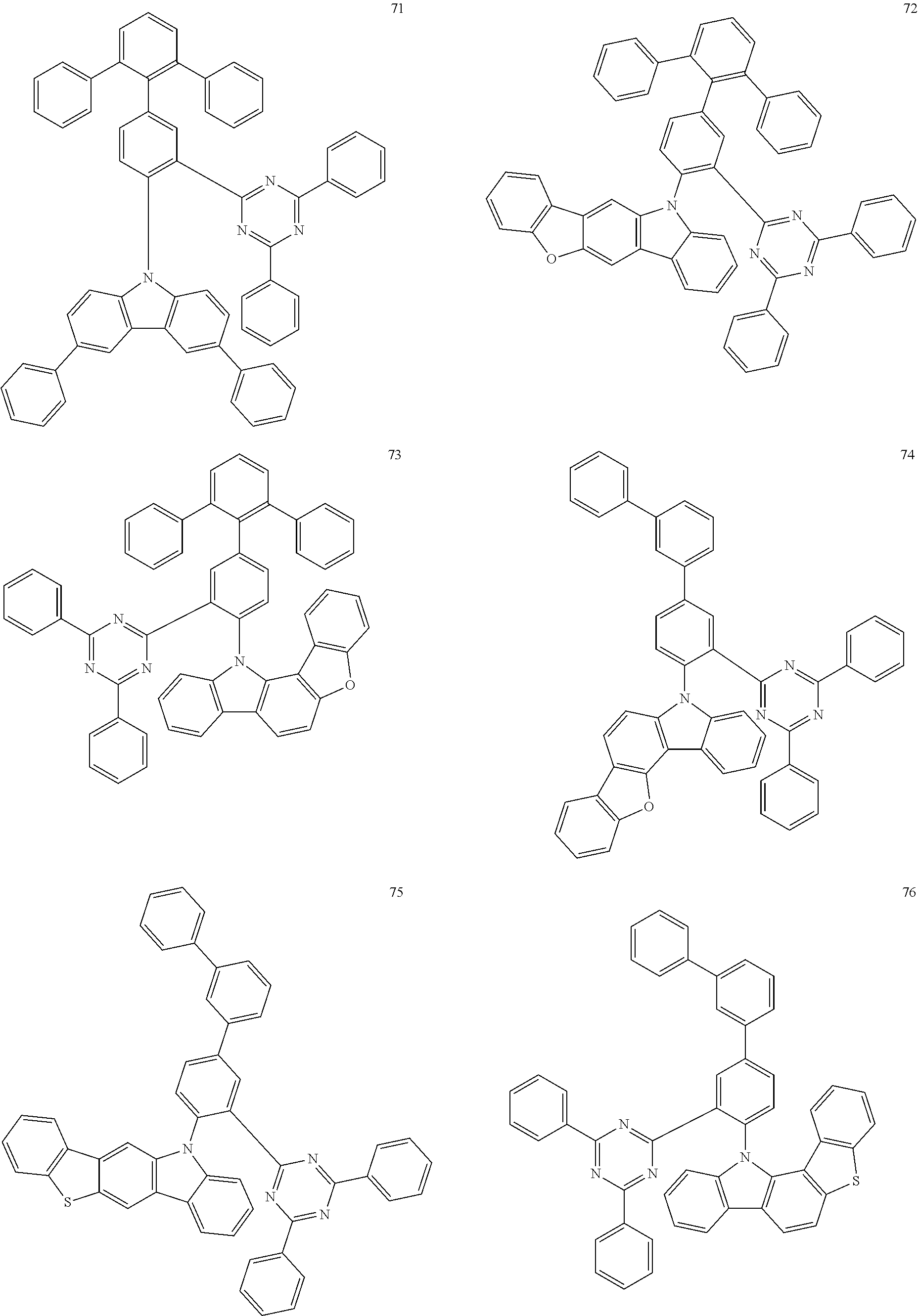

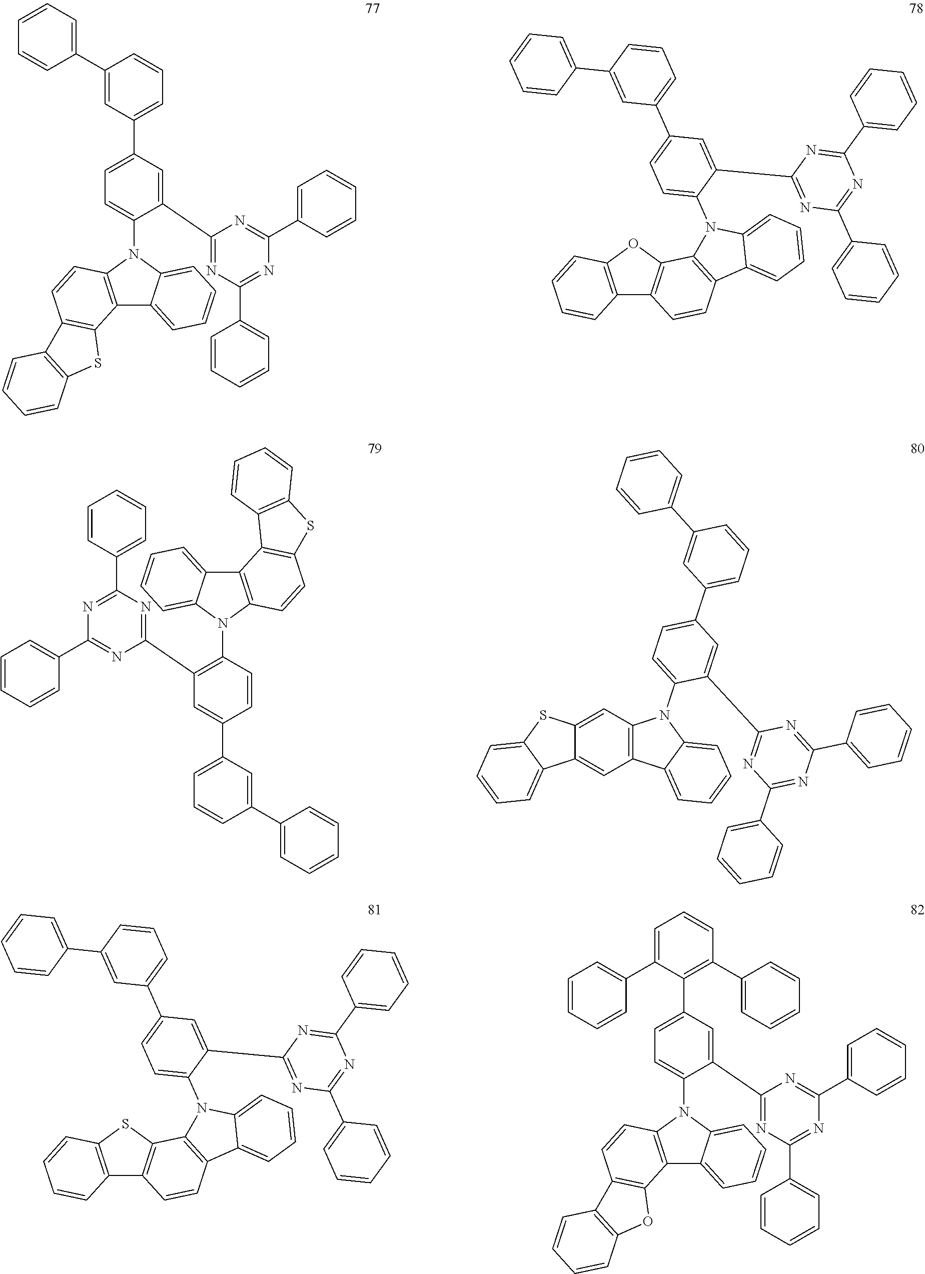

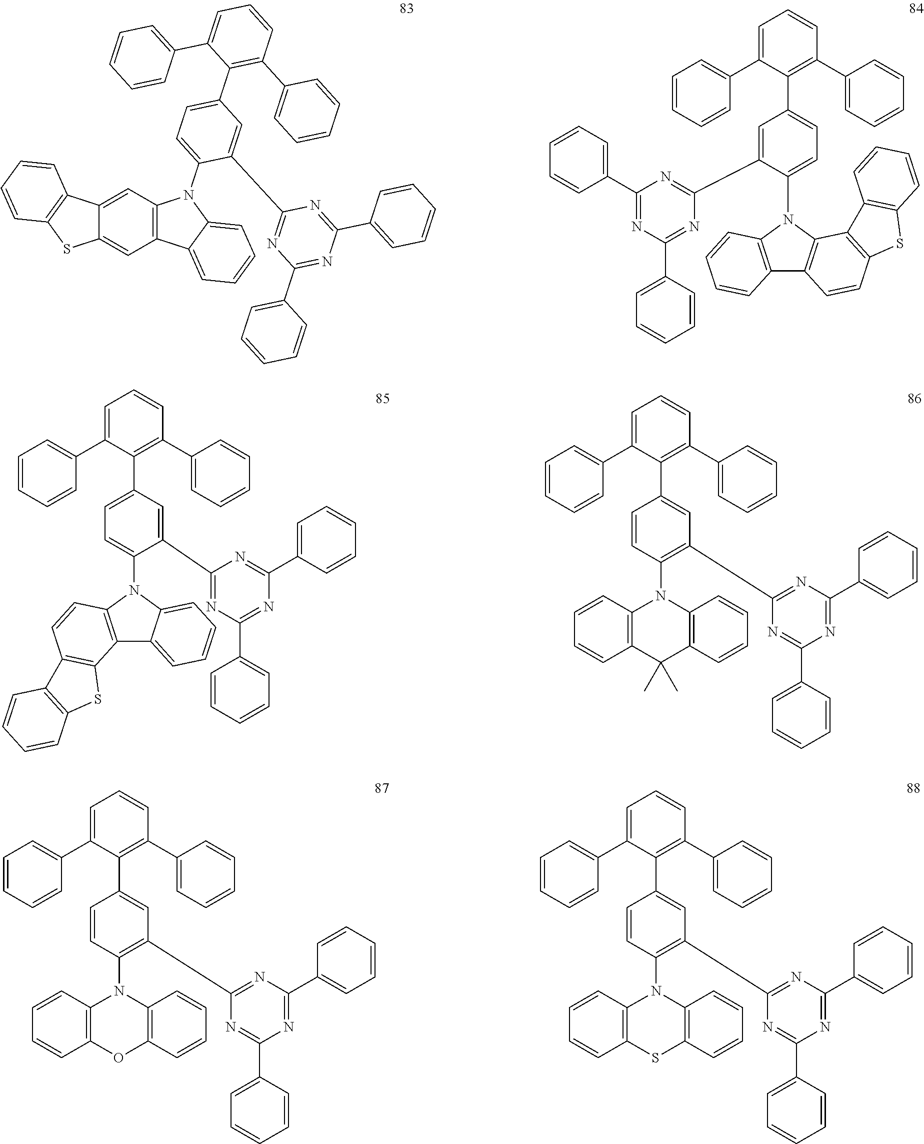

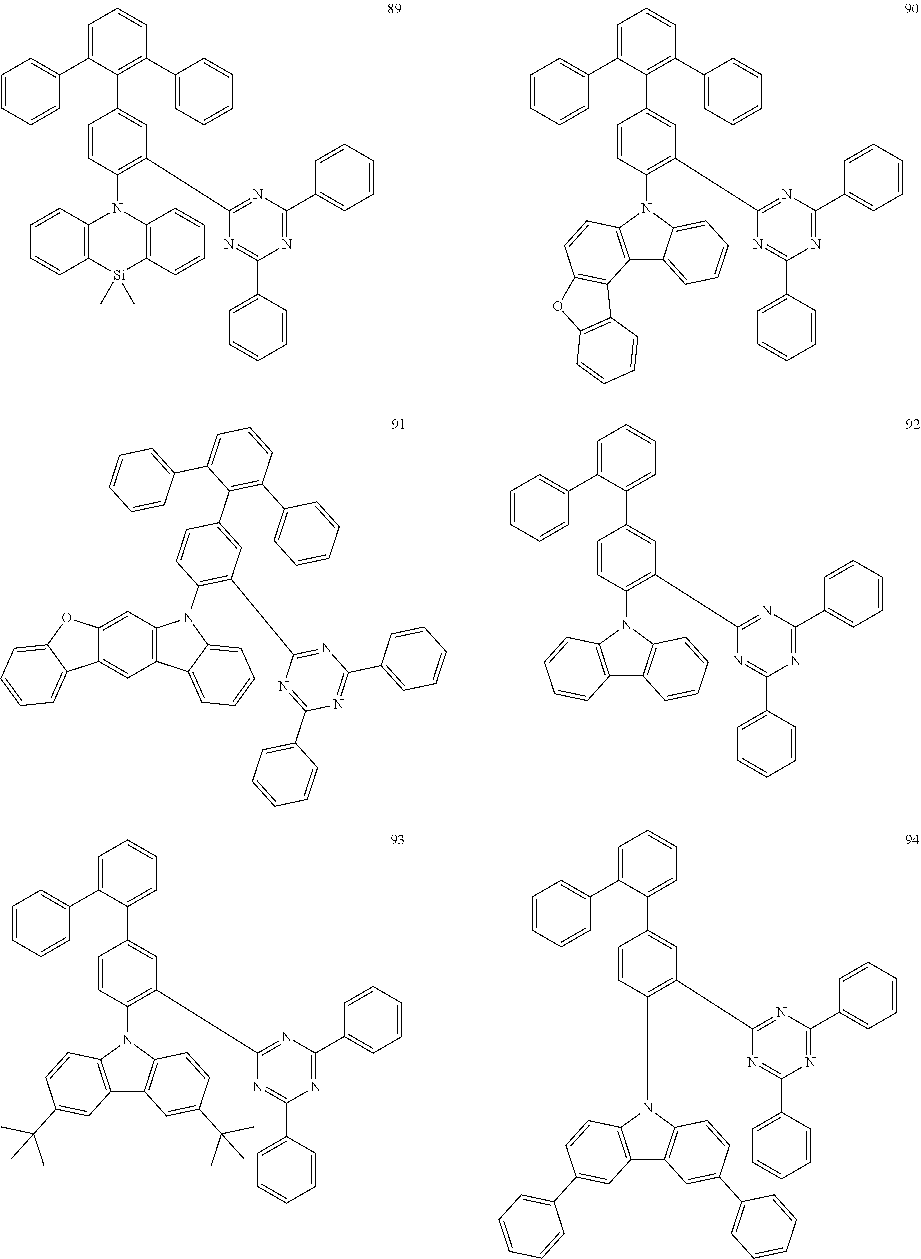

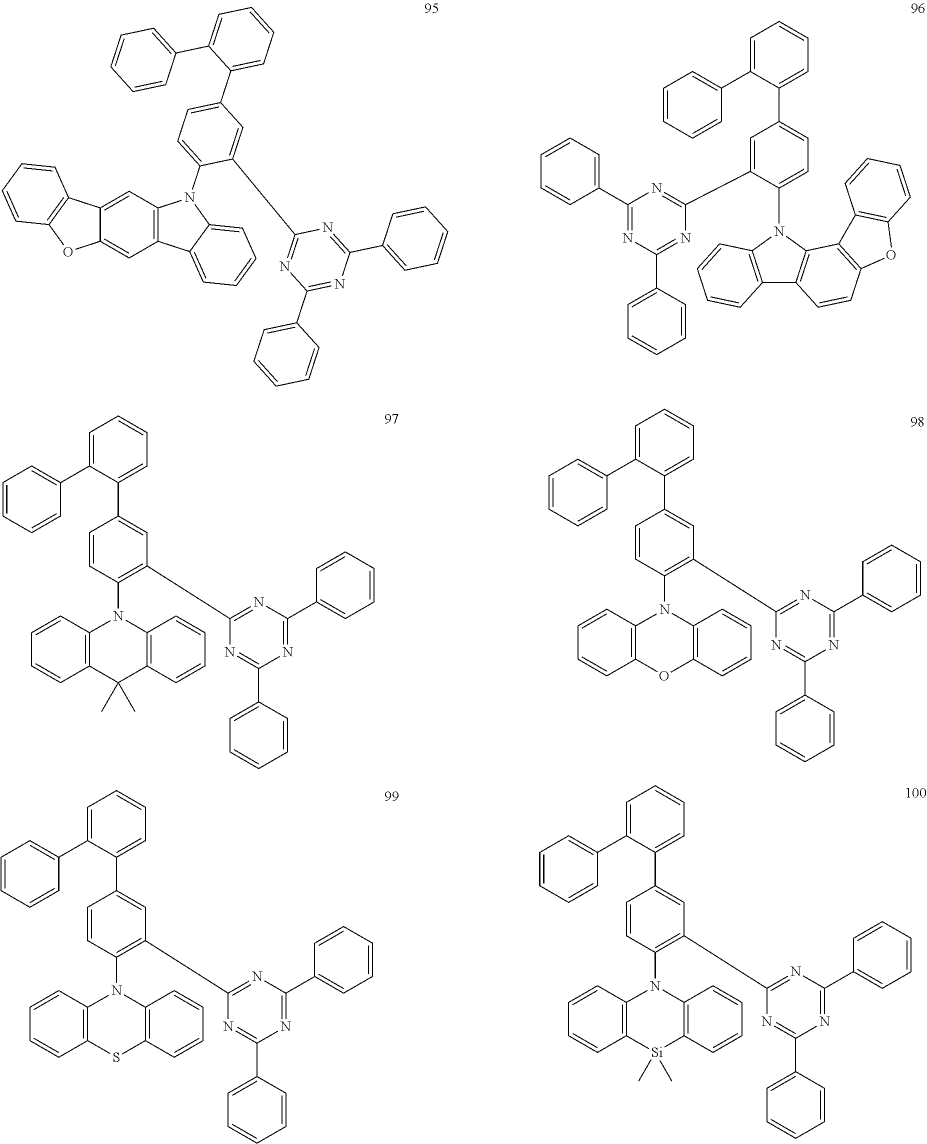

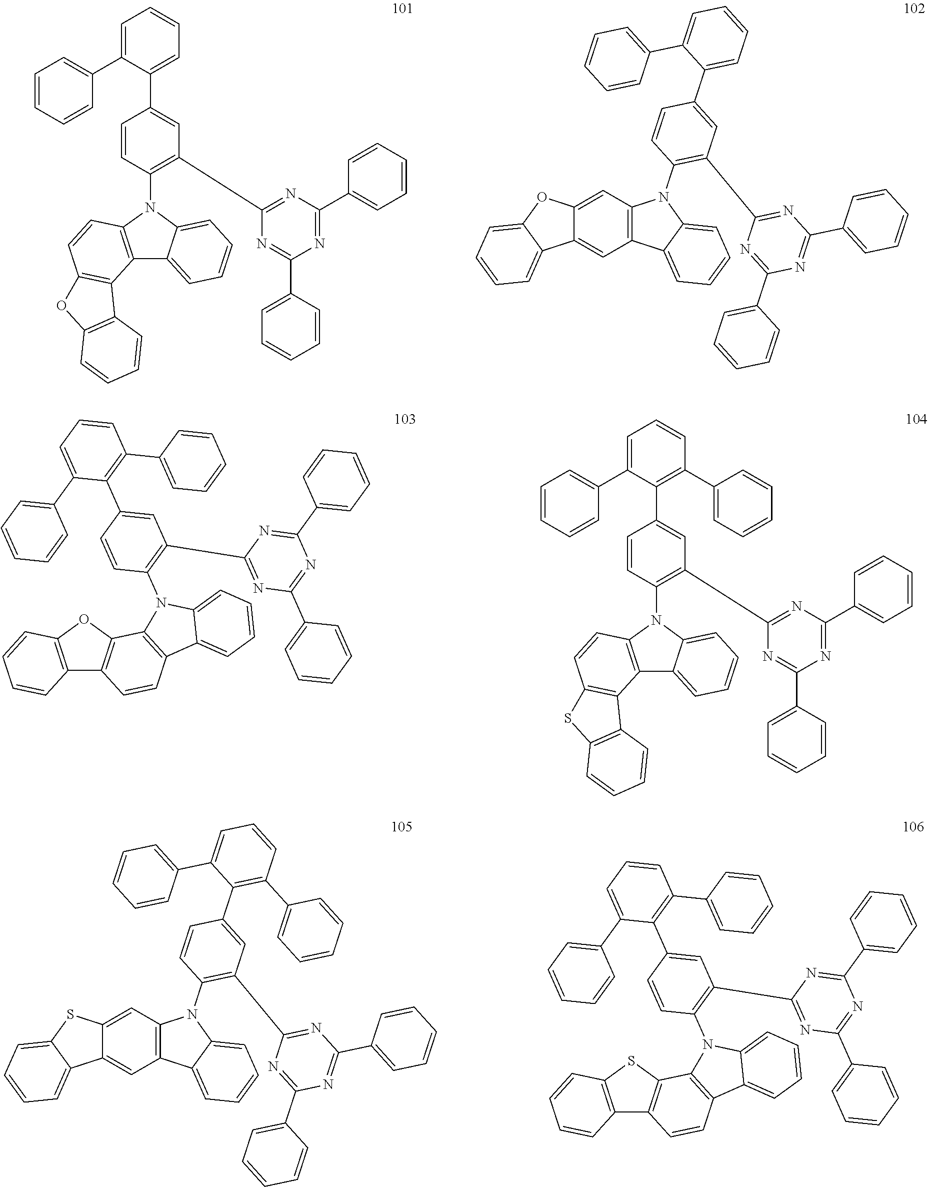

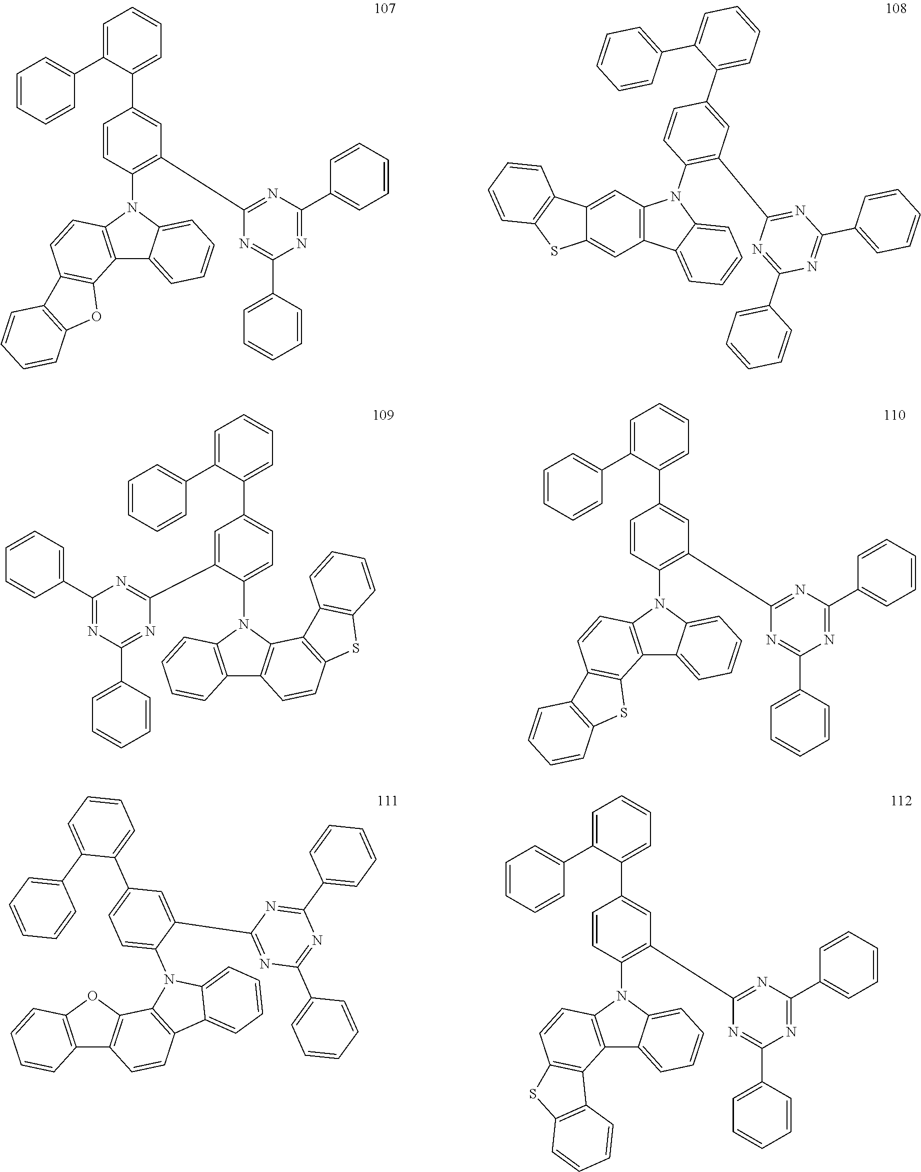

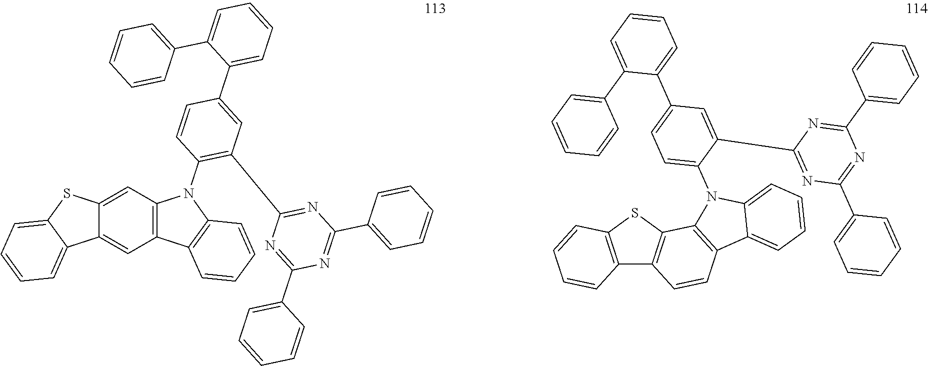 [SMILES: c1ccc(-c2nc(-c3ccccc3)nc(-c3cc(-c4ccccc4-c4ccccc4)ccc3-n3c4ccccc4c4cc5c(cc43)sc3ccccc35)n2)cc1 ; c1ccc(-c2nc(-c3ccccc3)nc(-c3cc(-c4ccccc4-c4ccccc4)ccc3-n3c4ccccc4c4ccc5c6ccccc6sc5c43)n2)cc1]

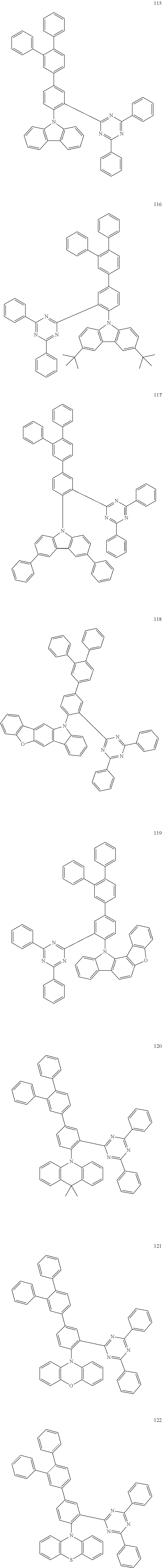

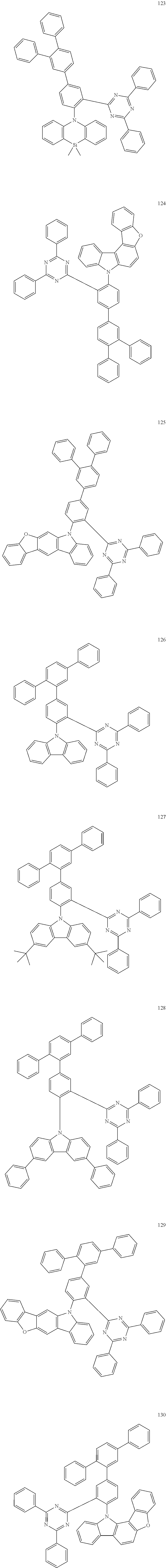

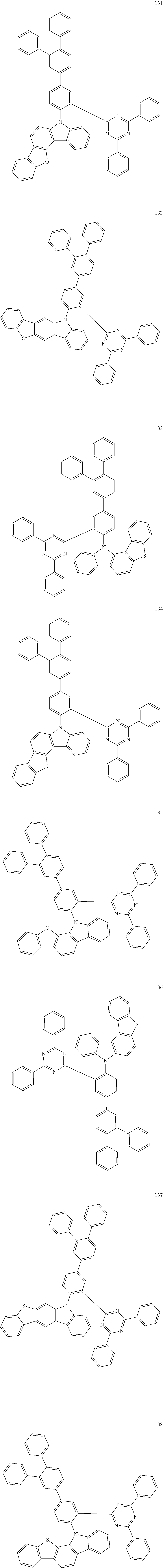

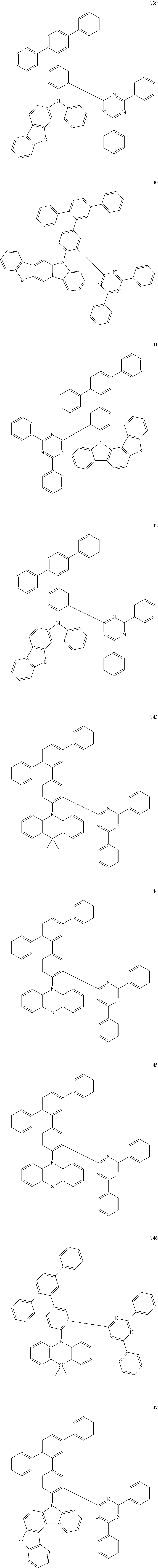

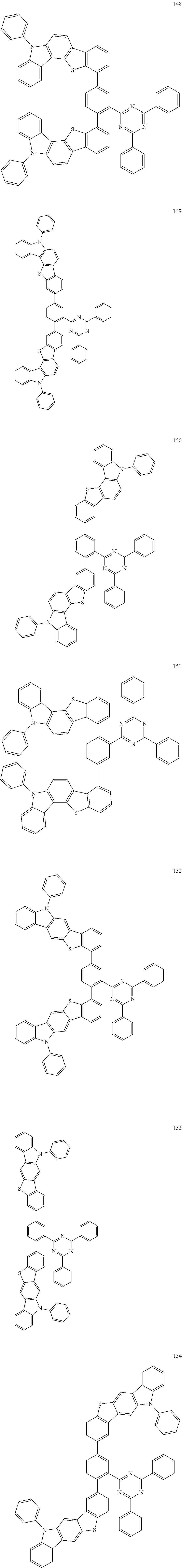

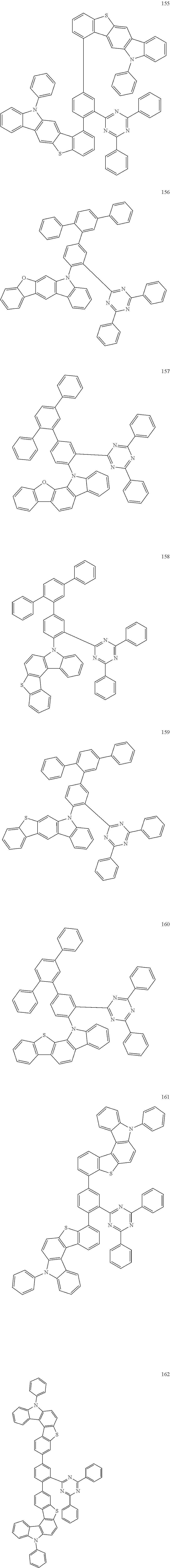

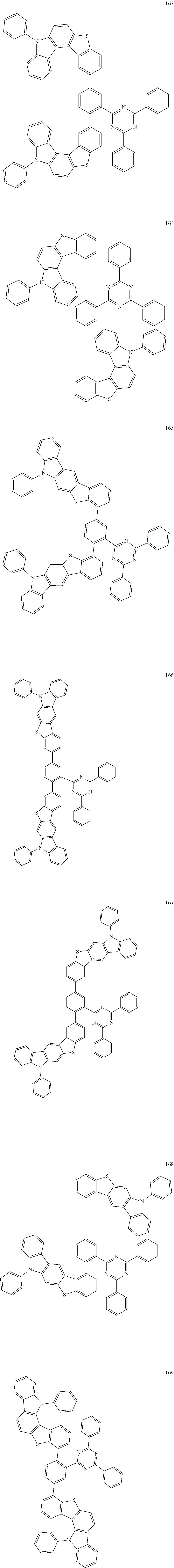

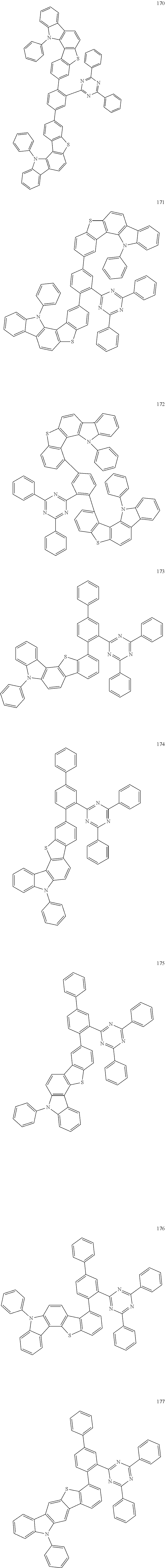

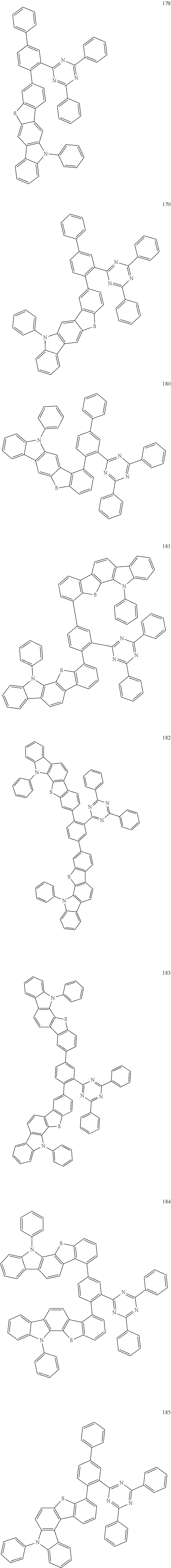

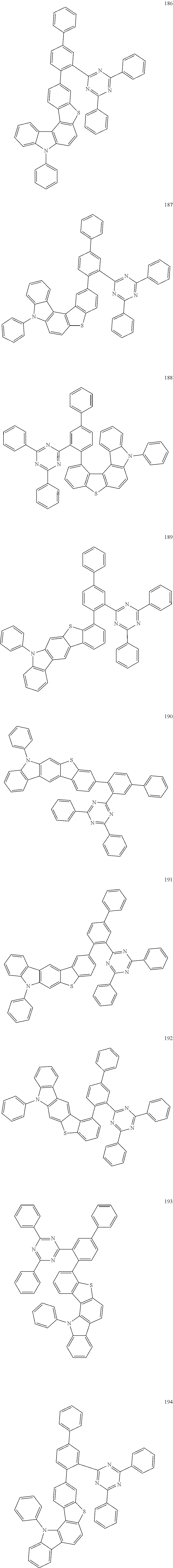

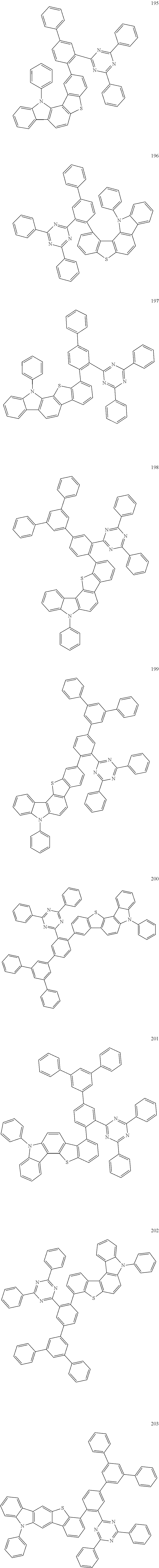

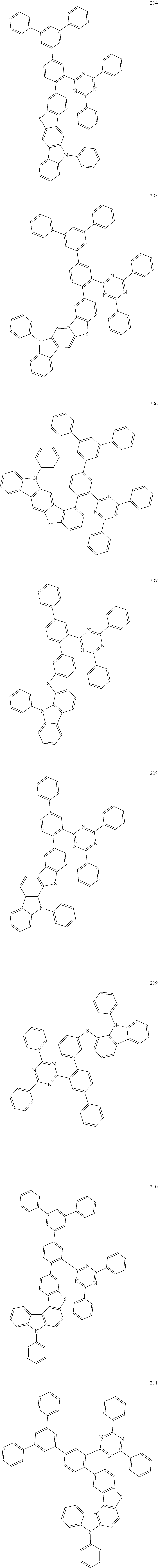

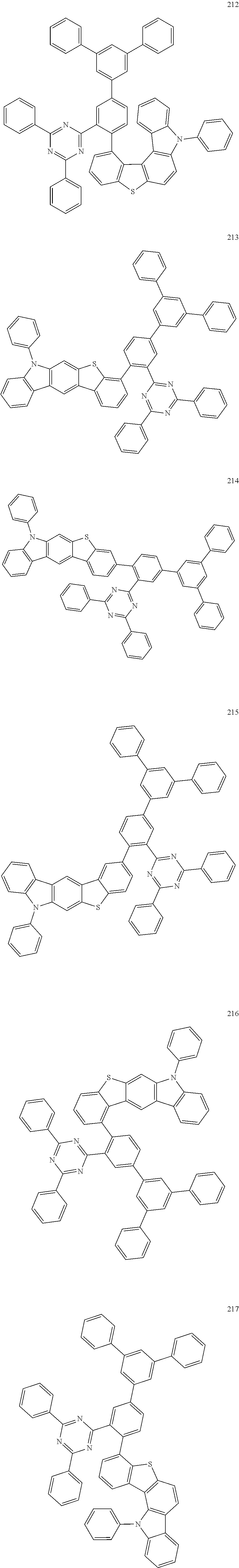

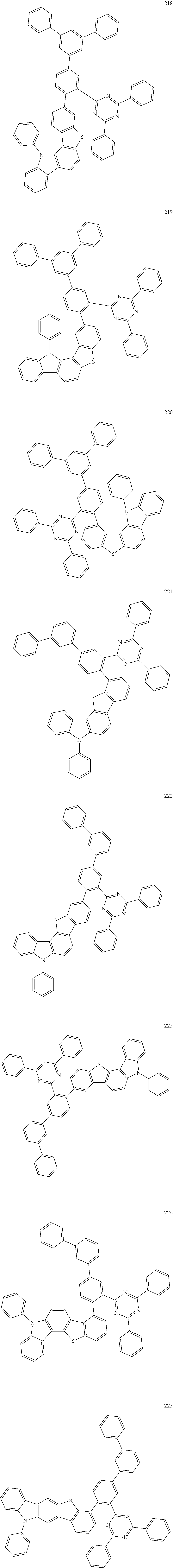

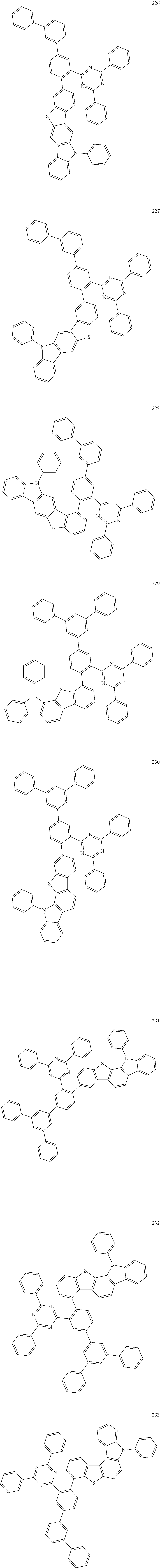

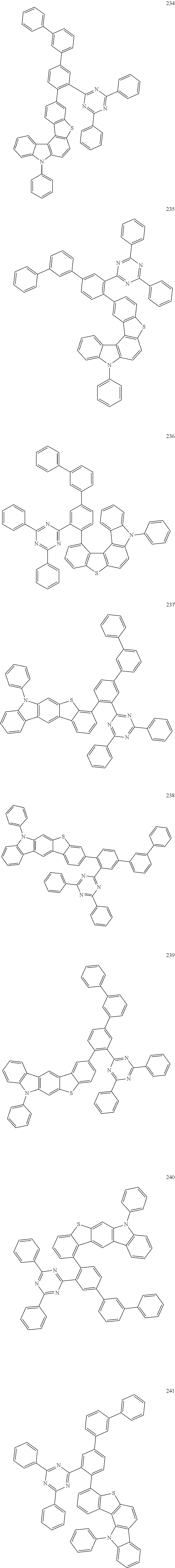

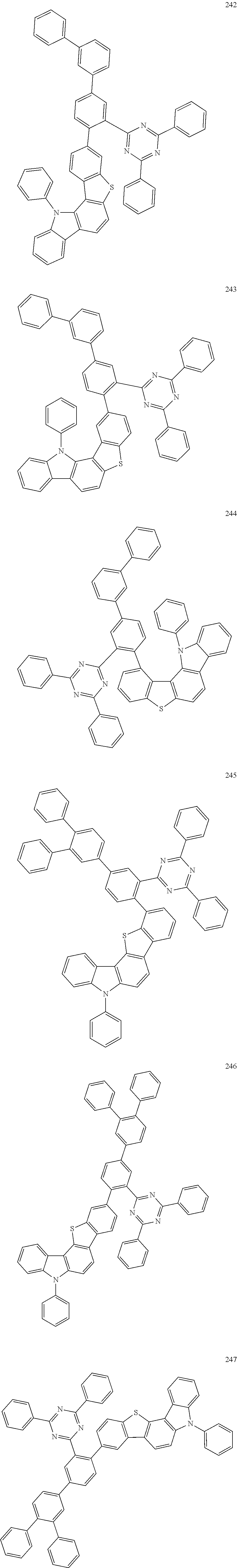

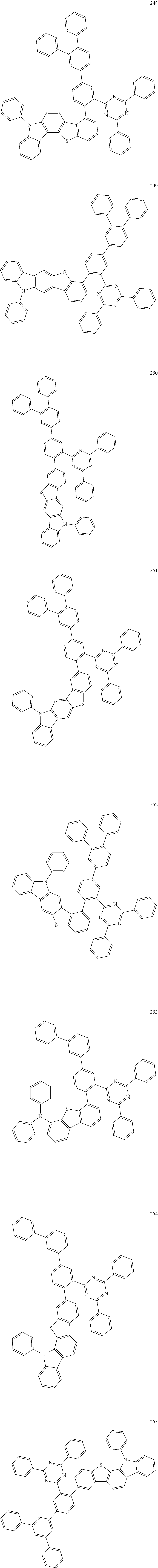

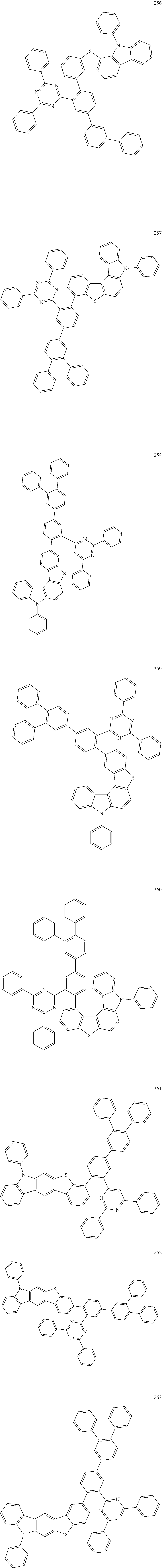

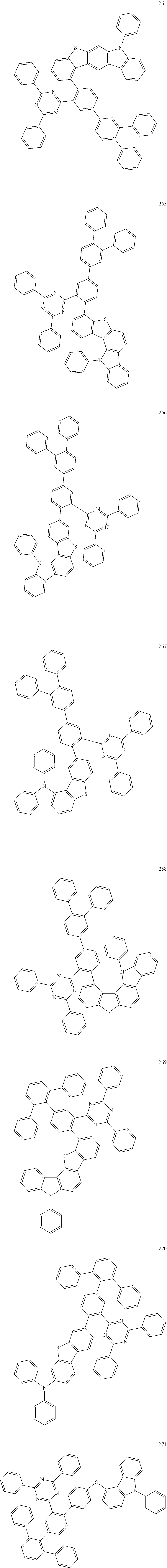

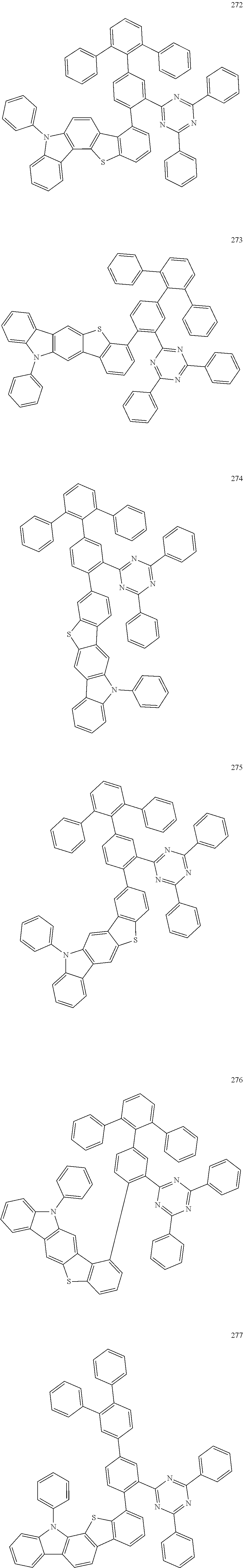

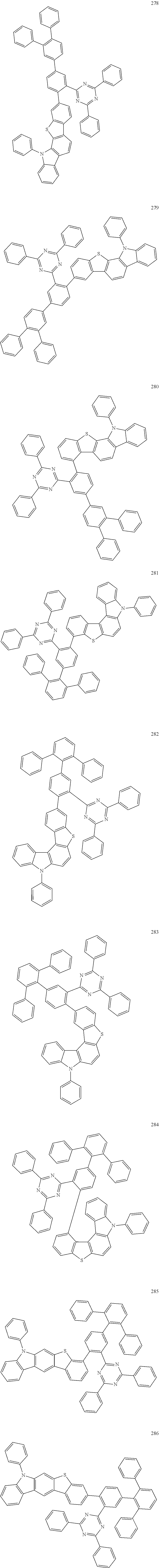

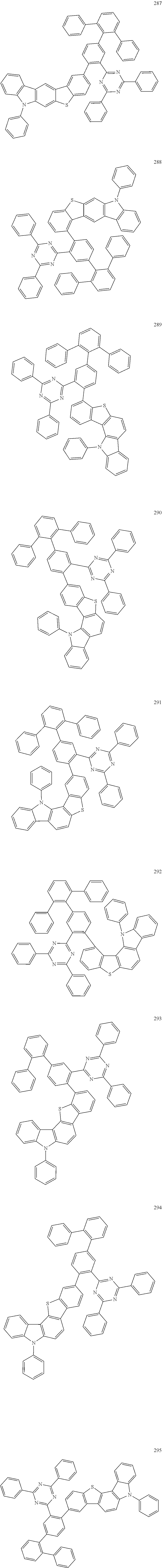

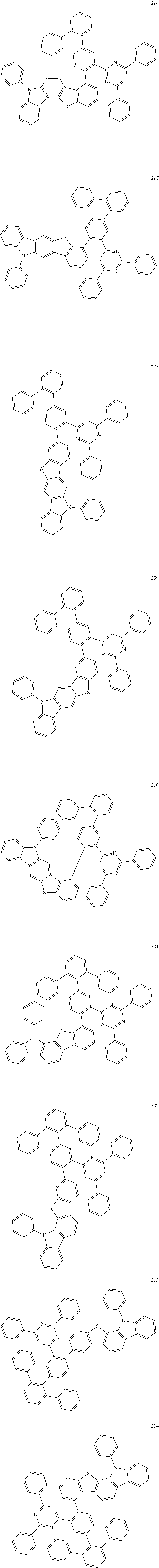

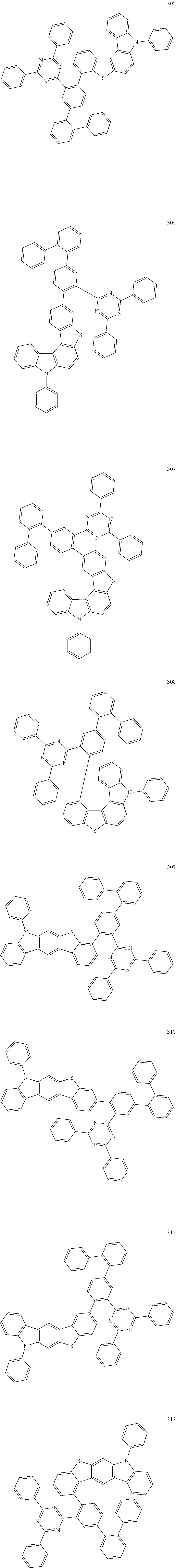

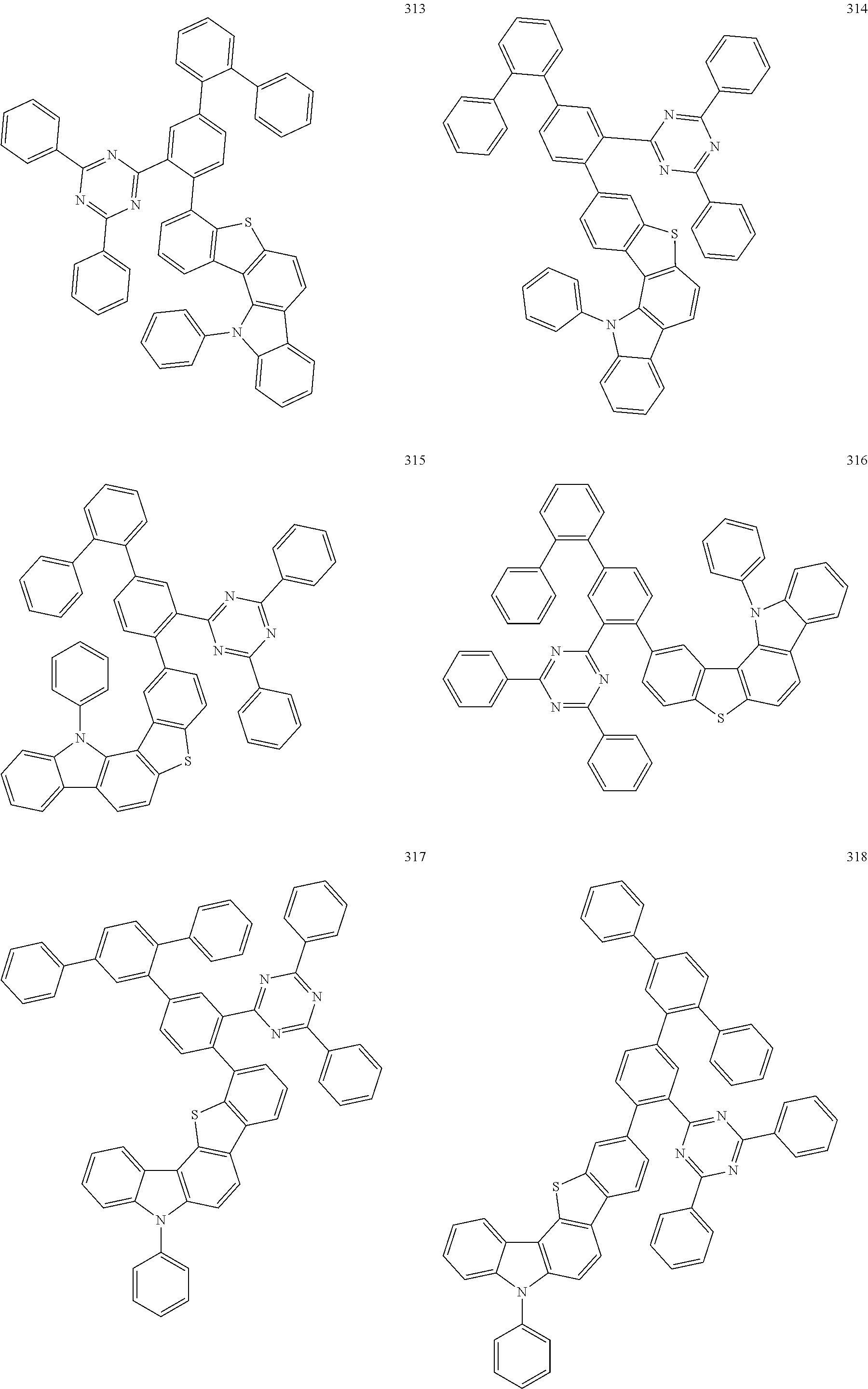

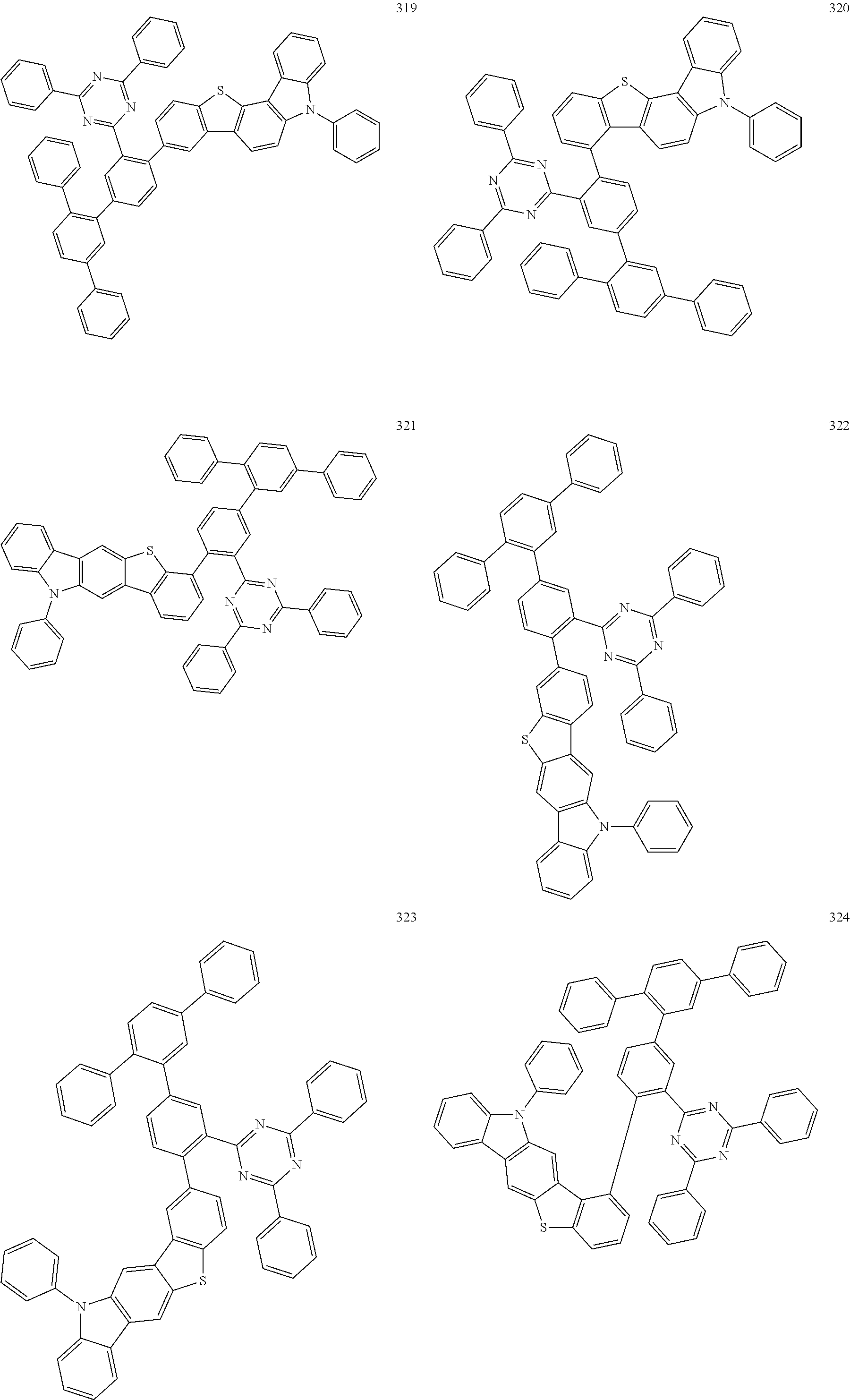

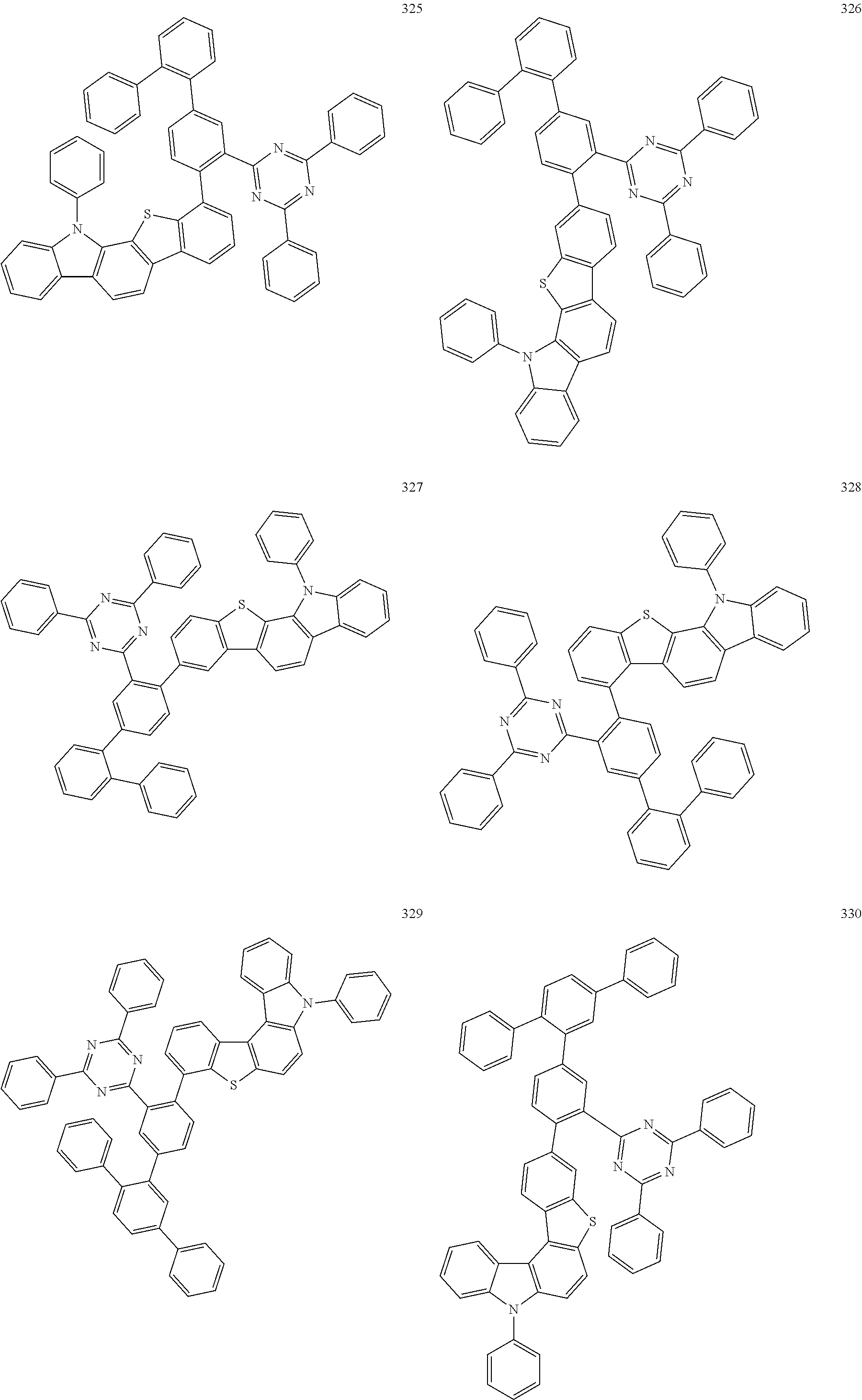

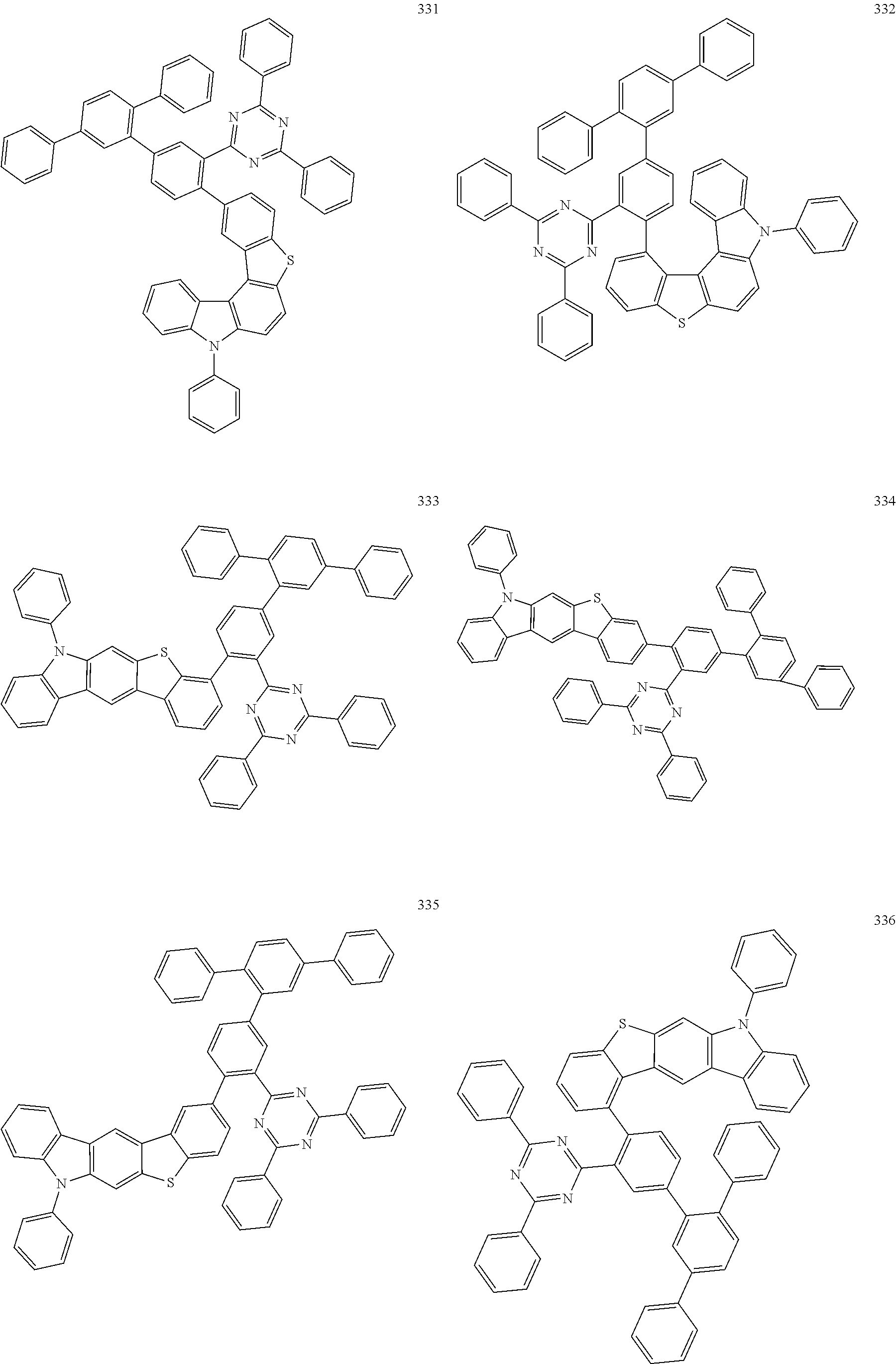

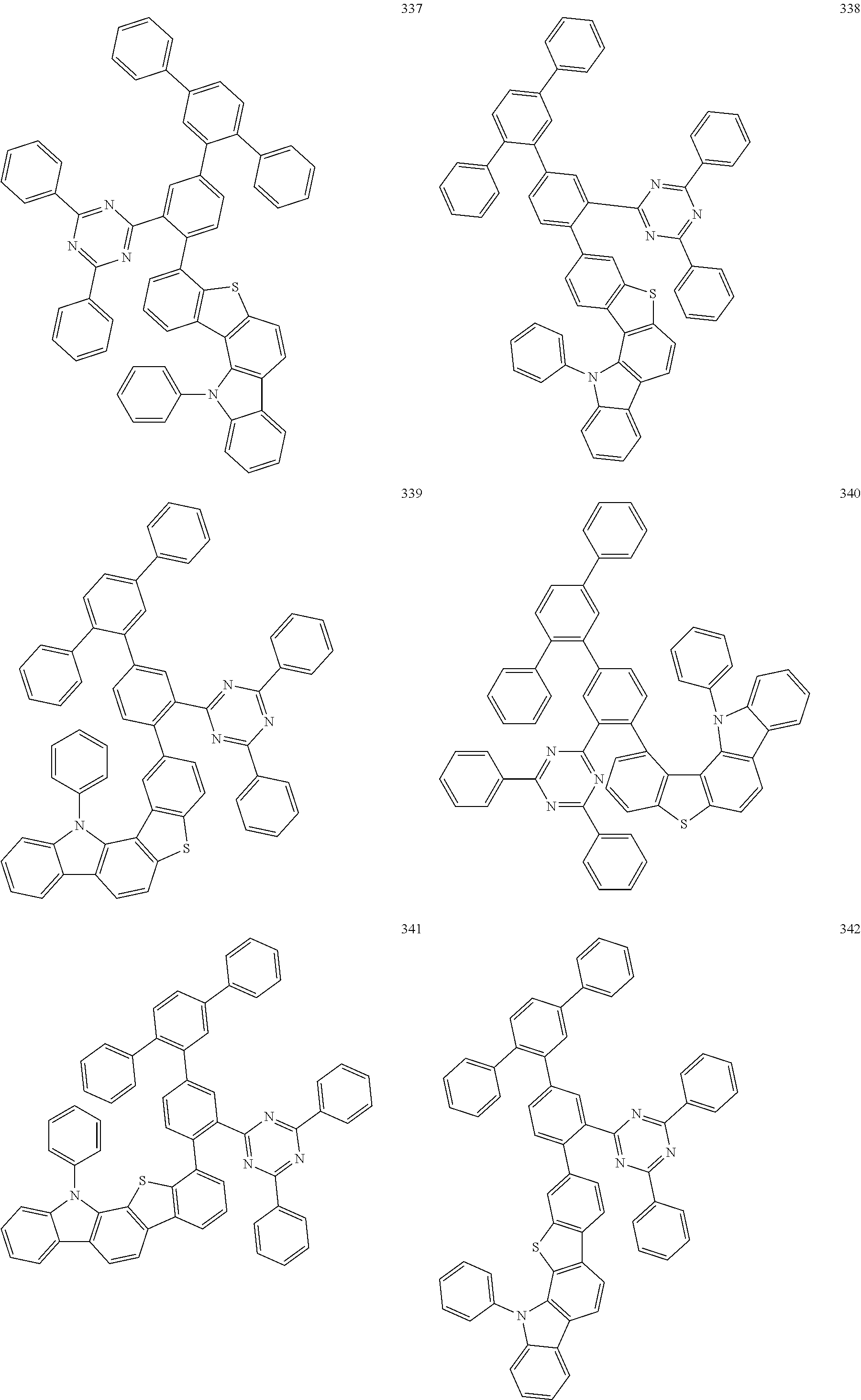

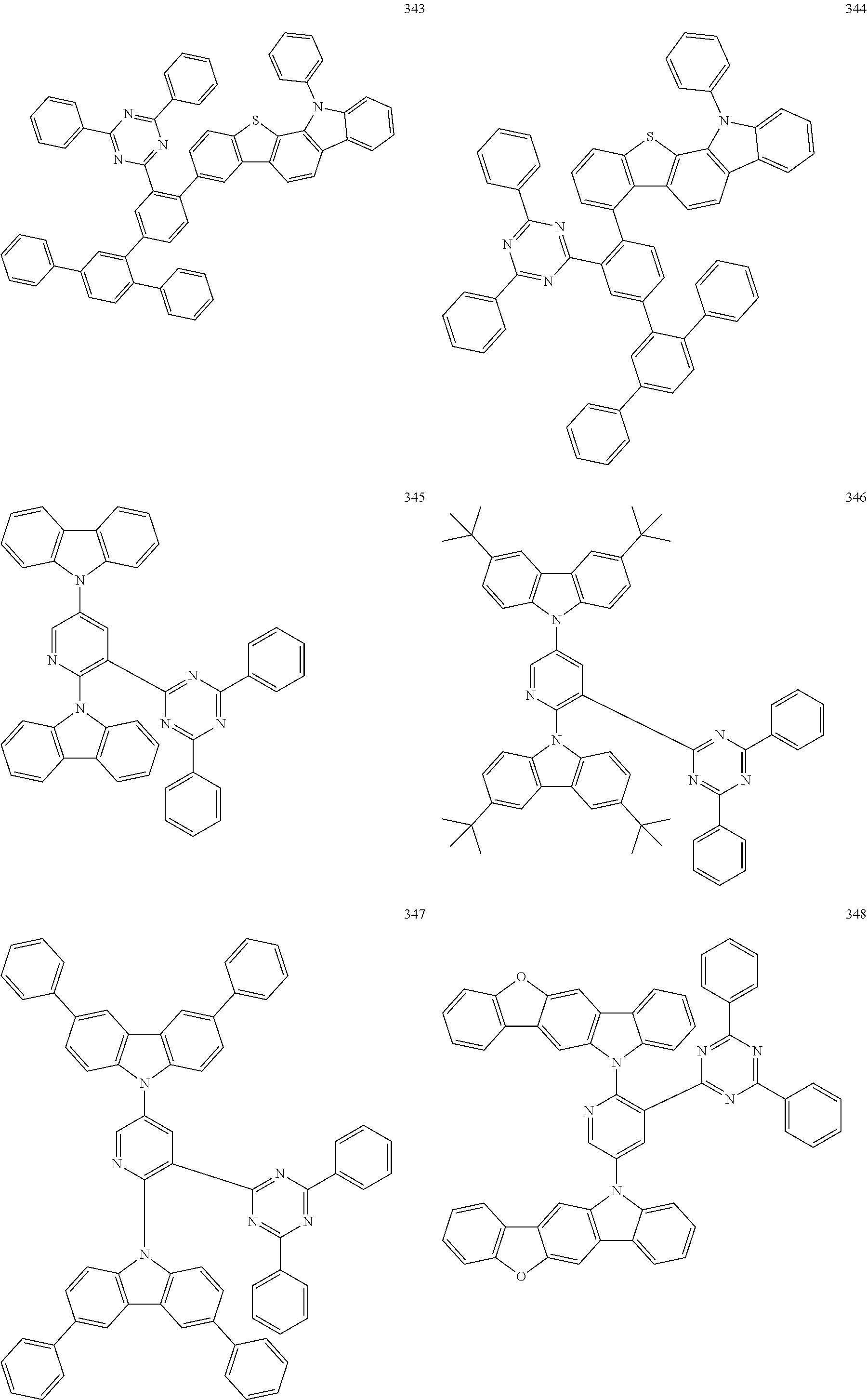

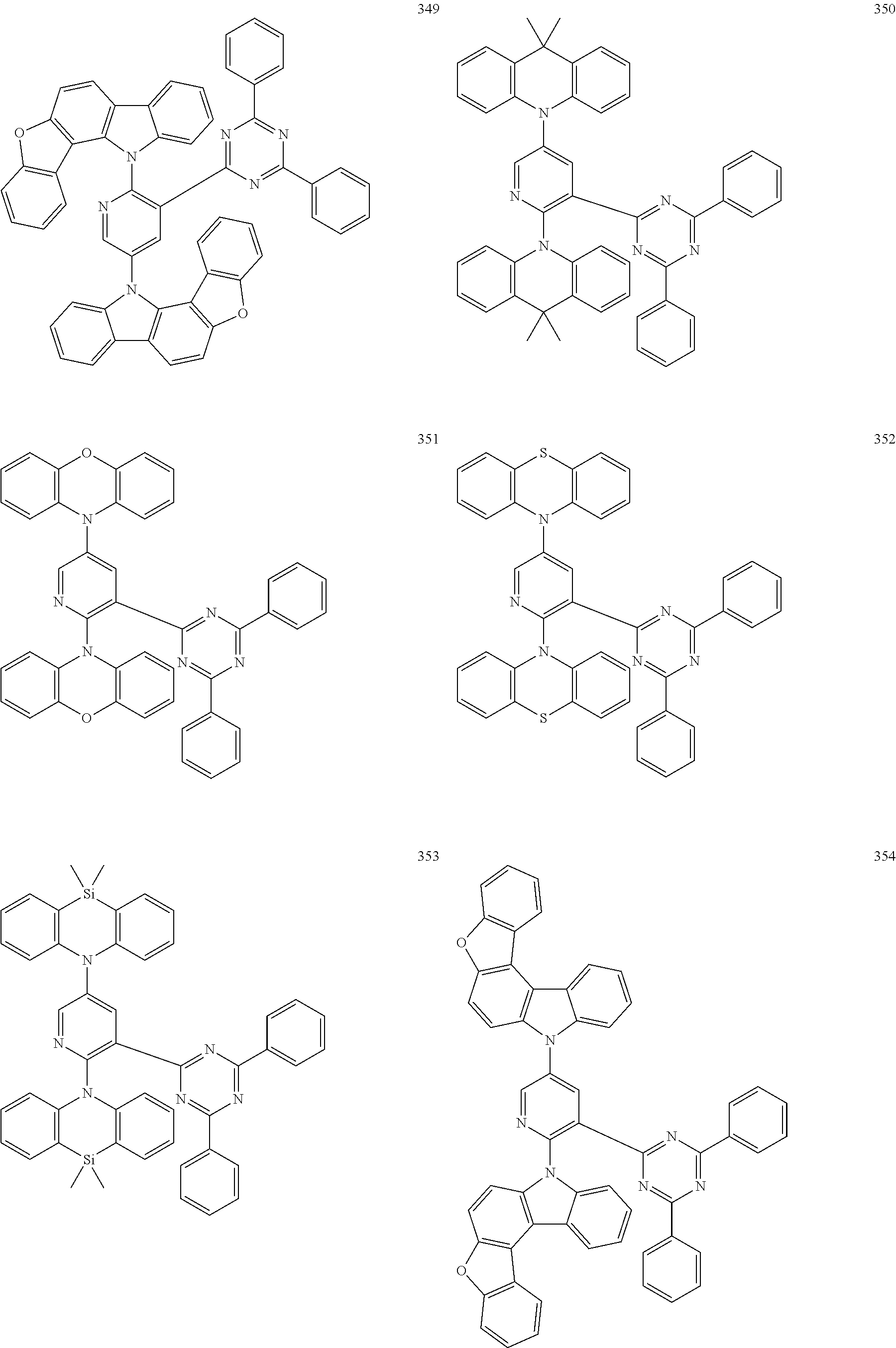

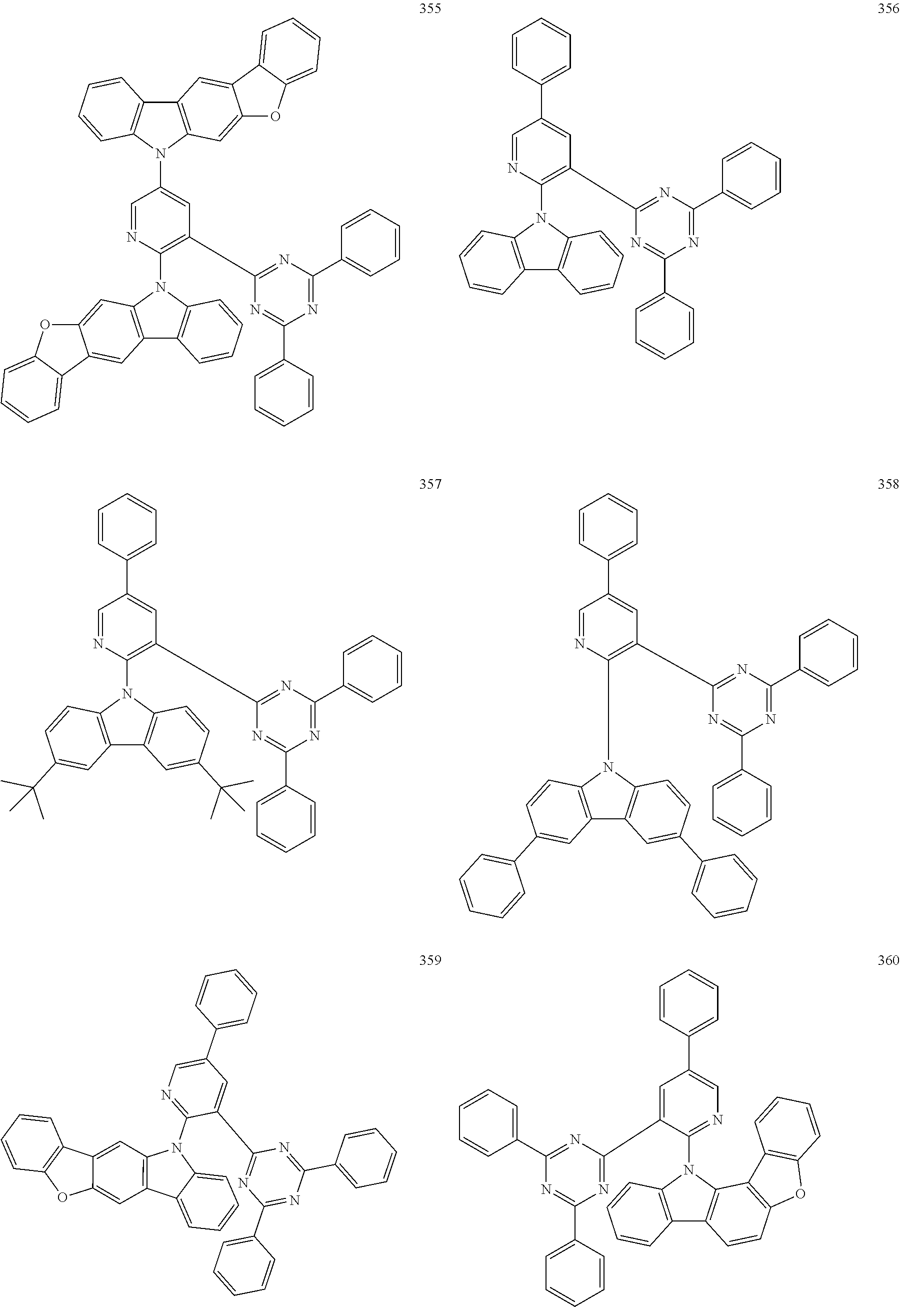

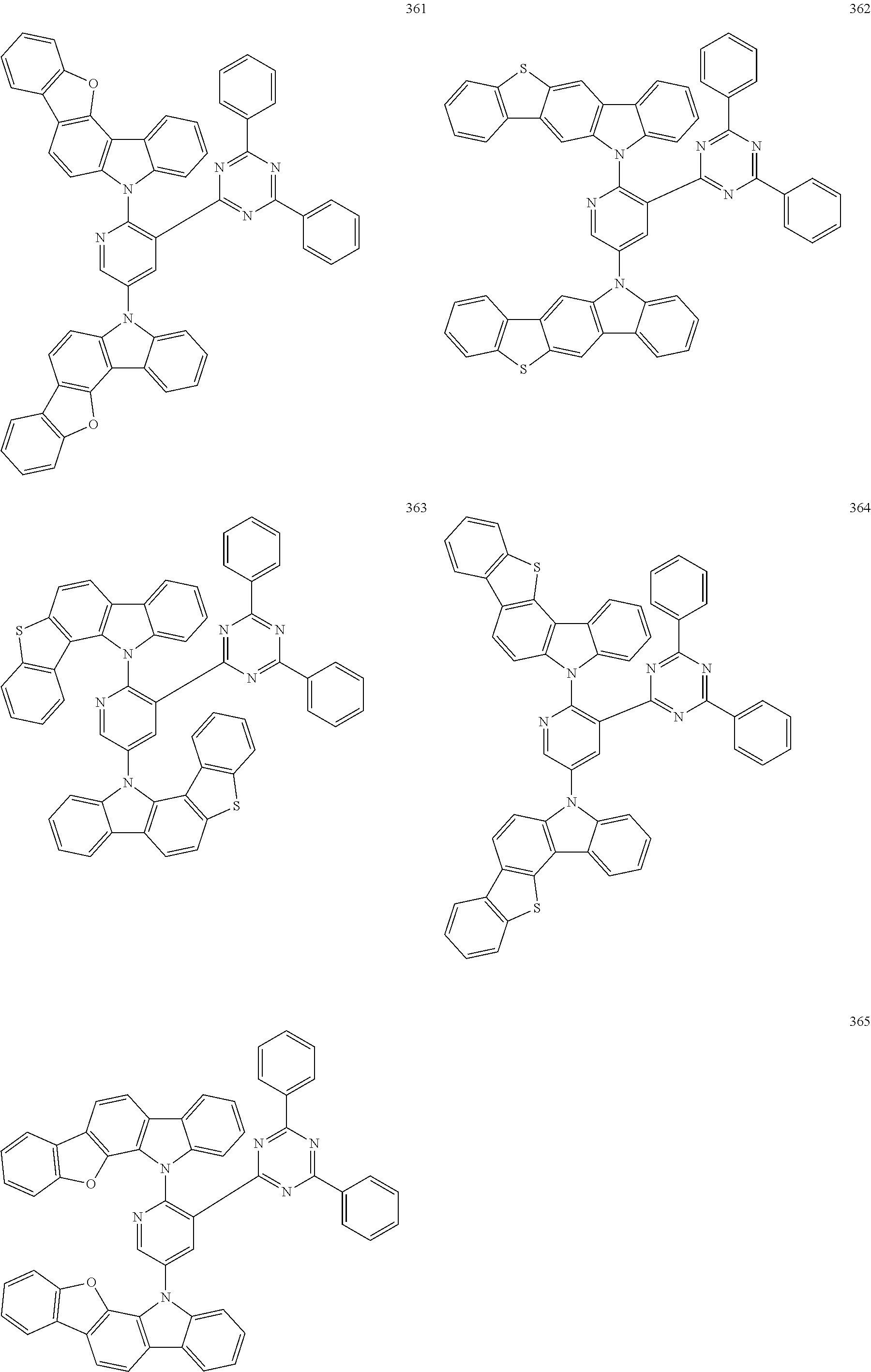

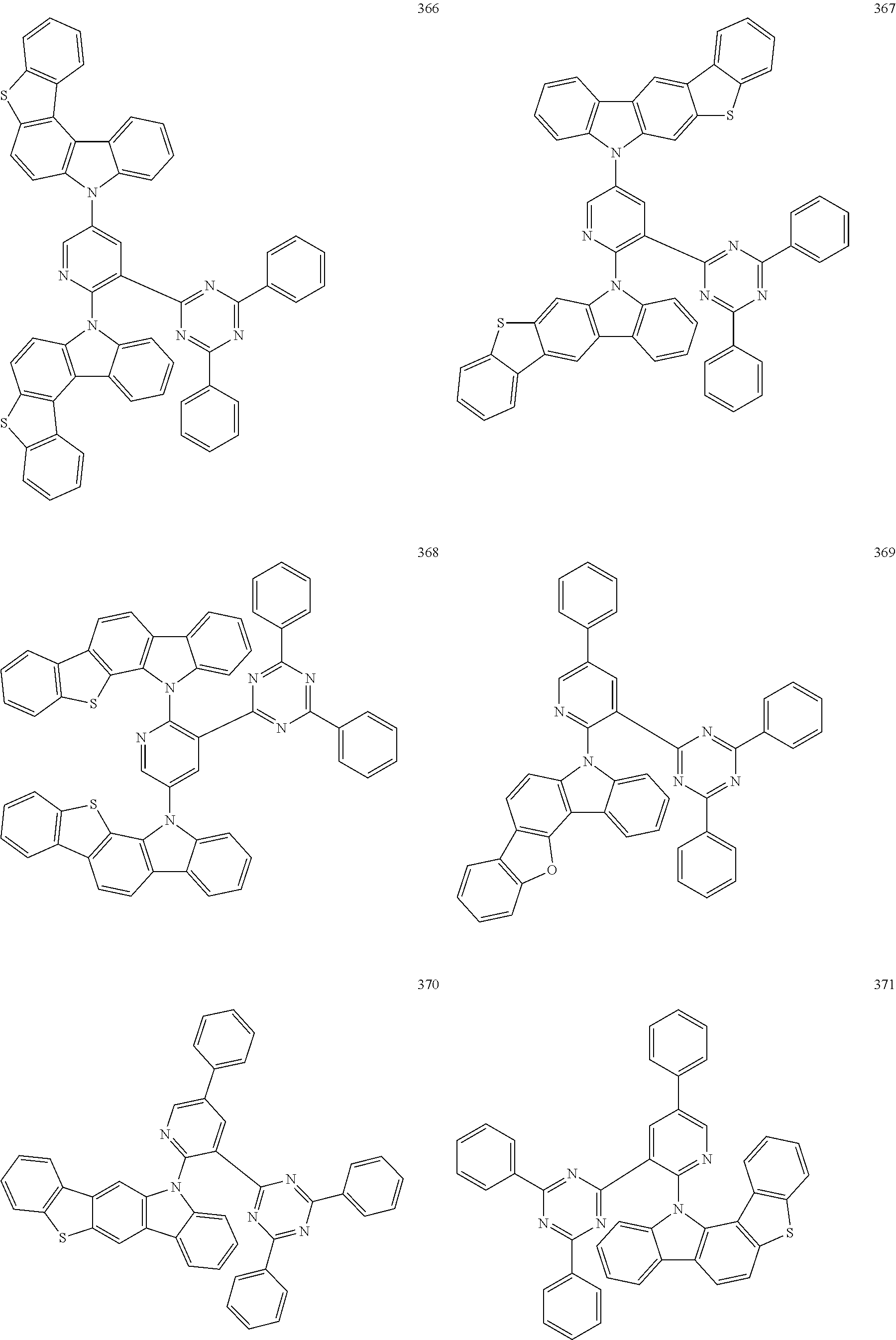

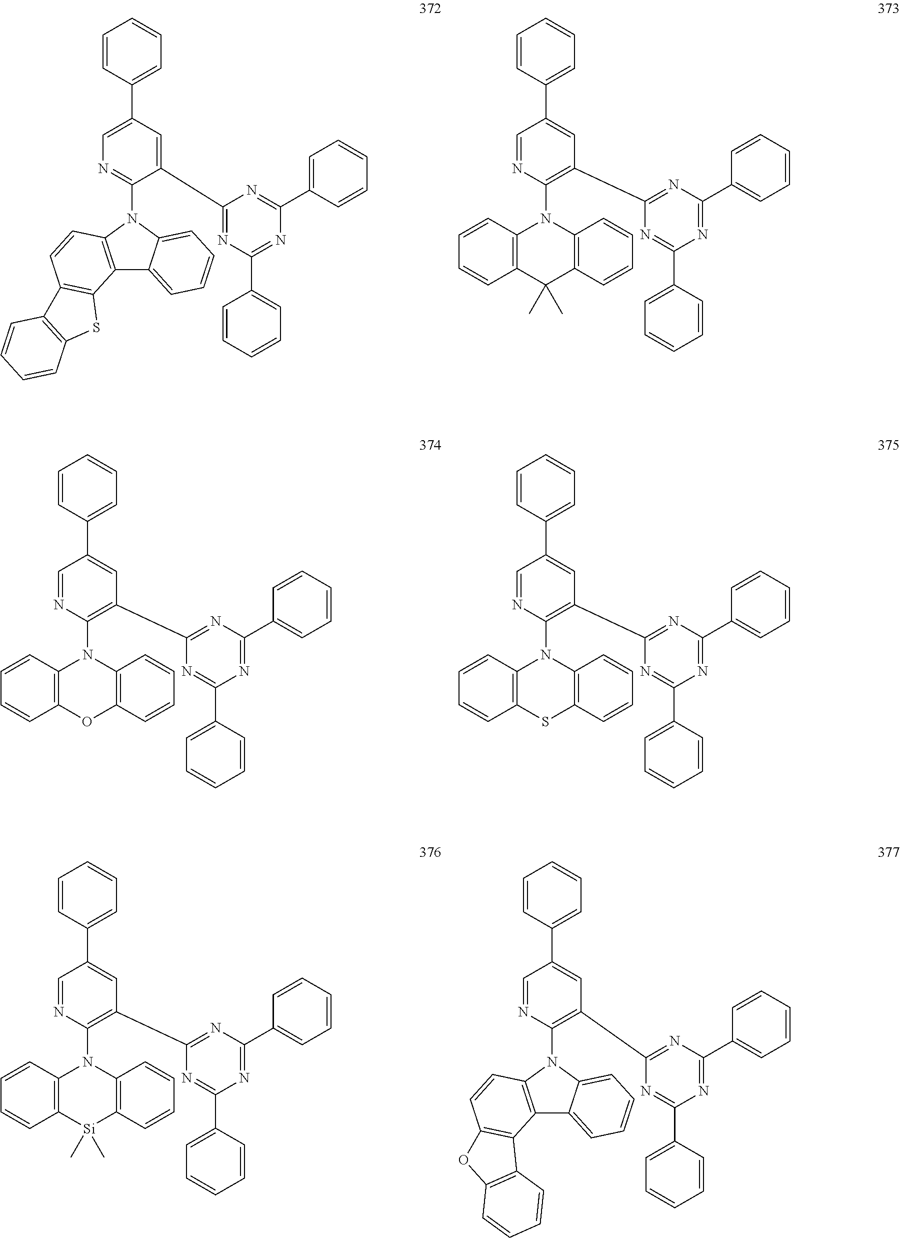 [SMILES: c1ccc(-c2cnc(-n3c4ccccc4c4c5sc6ccccc6c5ccc43)c(-c3nc(-c4ccccc4)nc(-c4ccccc4)n3)c2)cc1 ; CC1(C)c2ccccc2N(c2ncc(-c3ccccc3)cc2-c2nc(-c3ccccc3)nc(-c3ccccc3)n2)c2ccccc21 ; c1ccc(-c2cnc(N3c4ccccc4Oc4ccccc43)c(-c3nc(-c4ccccc4)nc(-c4ccccc4)n3)c2)cc1 ; c1ccc(-c2cnc(N3c4ccccc4Sc4ccccc43)c(-c3nc(-c4ccccc4)nc(-c4ccccc4)n3)c2)cc1 ; C[Si]1(C)c2ccccc2N(c2ncc(-c3ccccc3)cc2-c2nc(-c3ccccc3)nc(-c3ccccc3)n2)c2ccccc21 ; c1ccc(-c2cnc(-n3c4ccccc4c4c5c(ccc43)oc3ccccc35)c(-c3nc(-c4ccccc4)nc(-c4ccccc4)n3)c2)cc1]

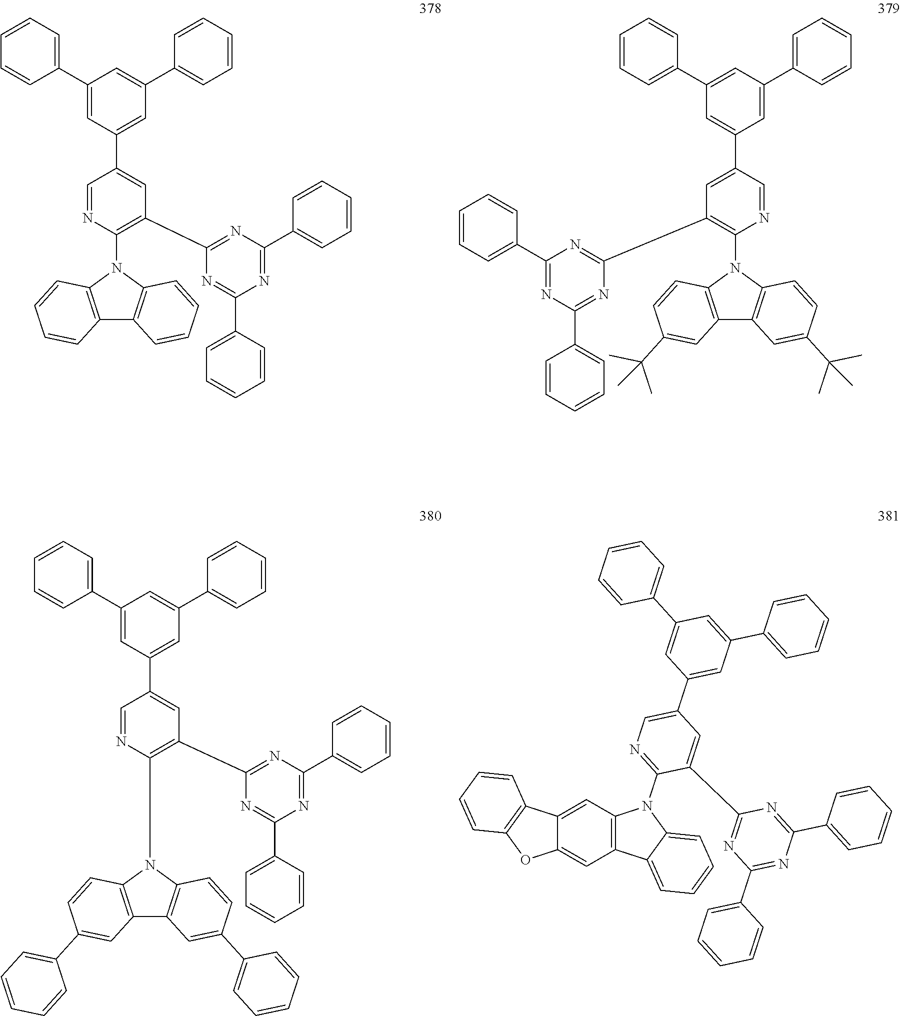 [SMILES: c1ccc(-c2cc(-c3ccccc3)cc(-c3cnc(-n4c5ccccc5c5ccccc54)c(-c4nc(-c5ccccc5)nc(-c5ccccc5)n4)c3)c2)cc1 ; CC(C)(C)c1ccc2c(c1)c1cc(C(C)(C)C)ccc1n2-c1ncc(-c2cc(-c3ccccc3)cc(-c3ccccc3)c2)cc1-c1nc(-c2ccccc2)nc(-c2ccccc2)n1 ; c1ccc(-c2cc(-c3ccccc3)cc(-c3cnc(-n4c5ccc(-c6ccccc6)cc5c5cc(-c6ccccc6)ccc54)c(-c4nc(-c5ccccc5)nc(-c5ccccc5)n4)c3)c2)cc1 ; c1ccc(-c2cc(-c3ccccc3)cc(-c3cnc(-n4c5ccccc5c5cc6oc7ccccc7c6cc54)c(-c4nc(-c5ccccc5)nc(-c5ccccc5)n4)c3)c2)cc1]

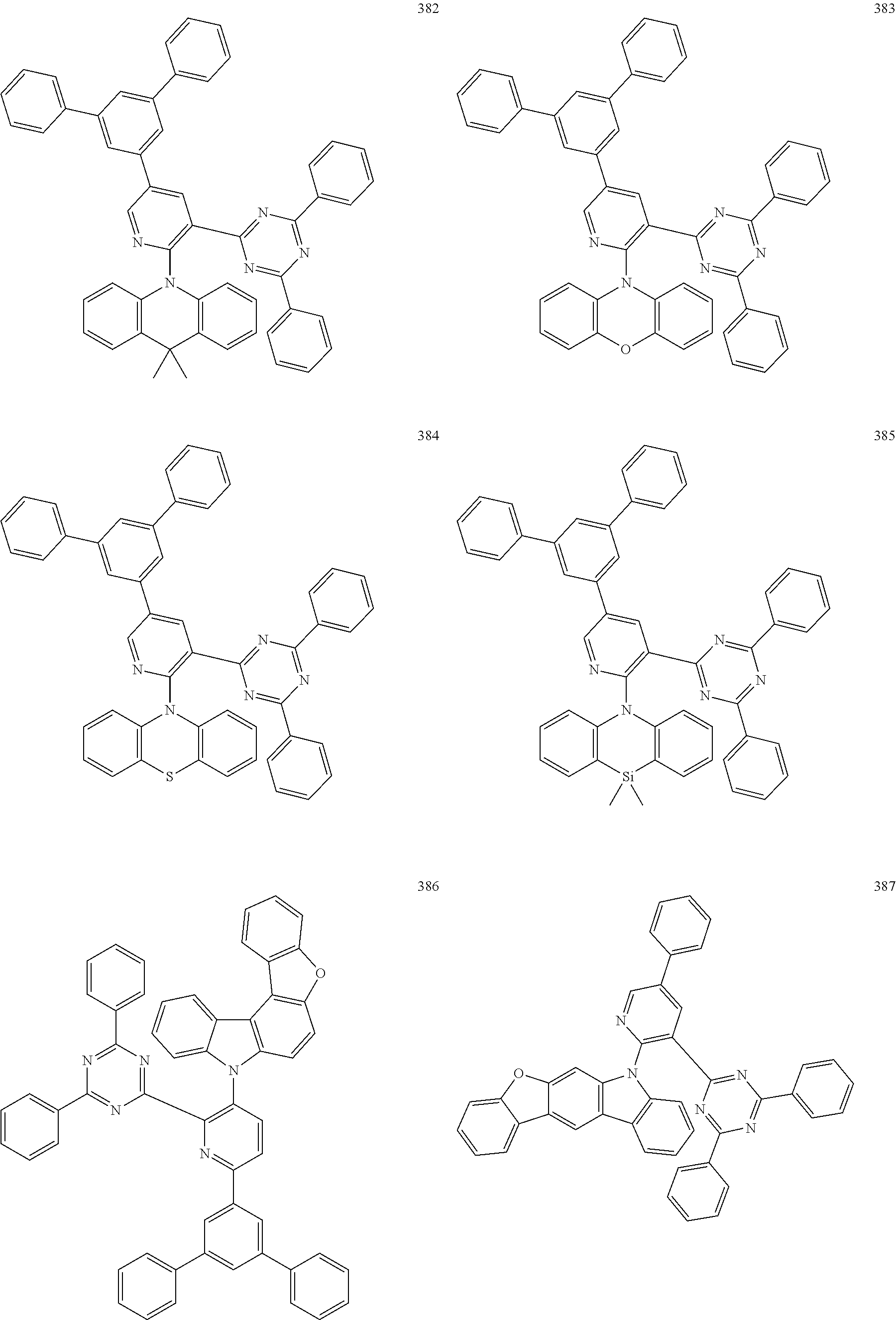

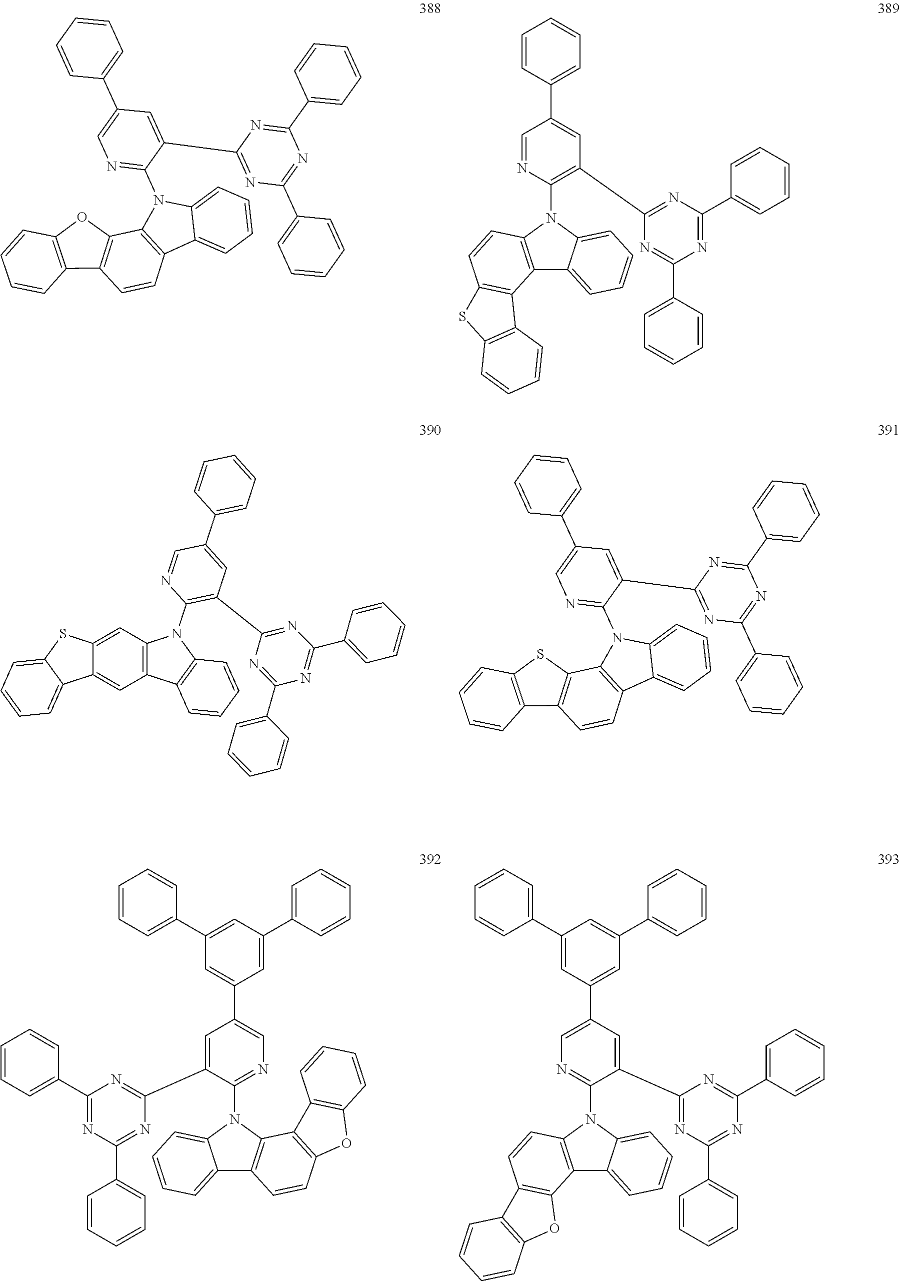

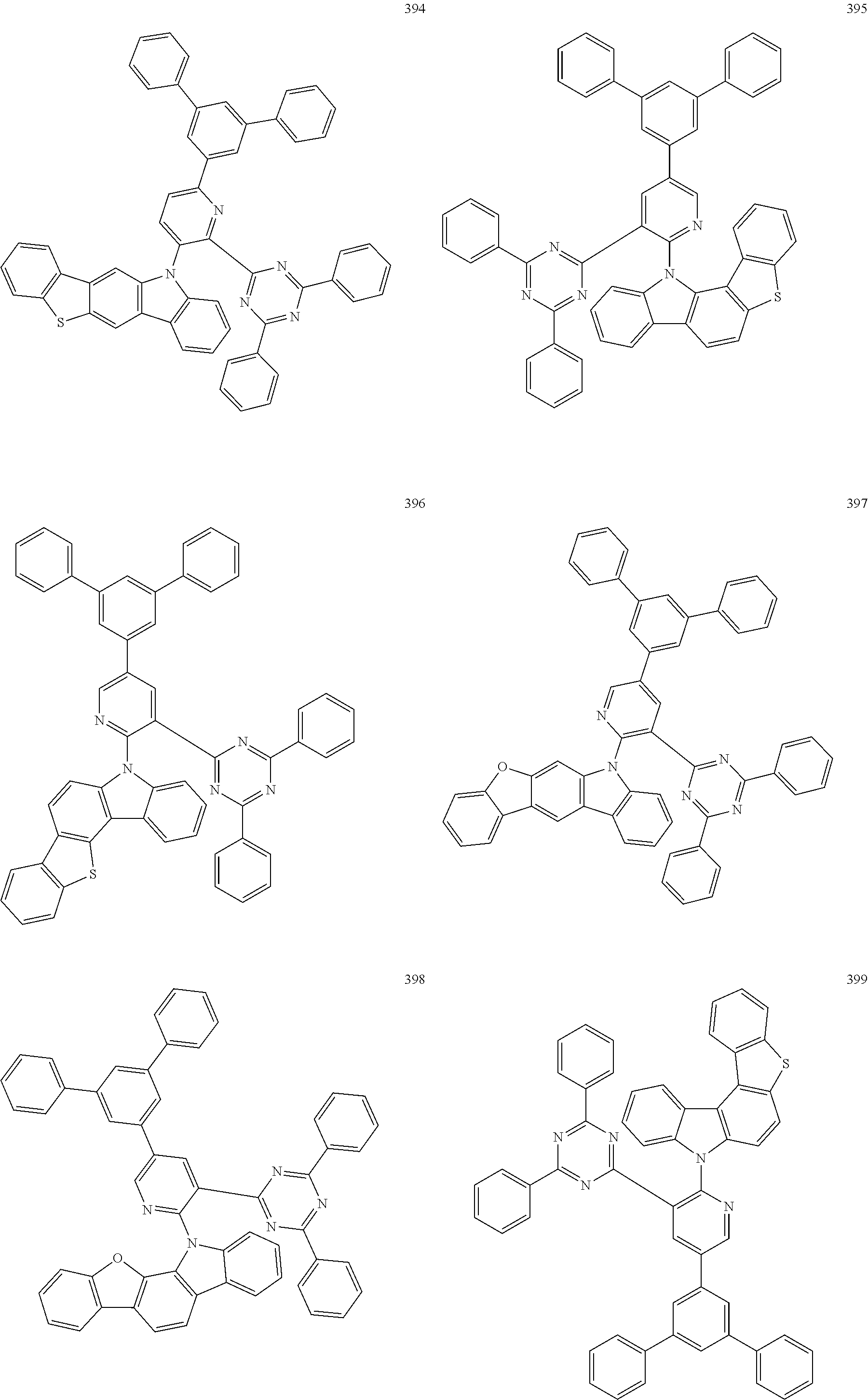

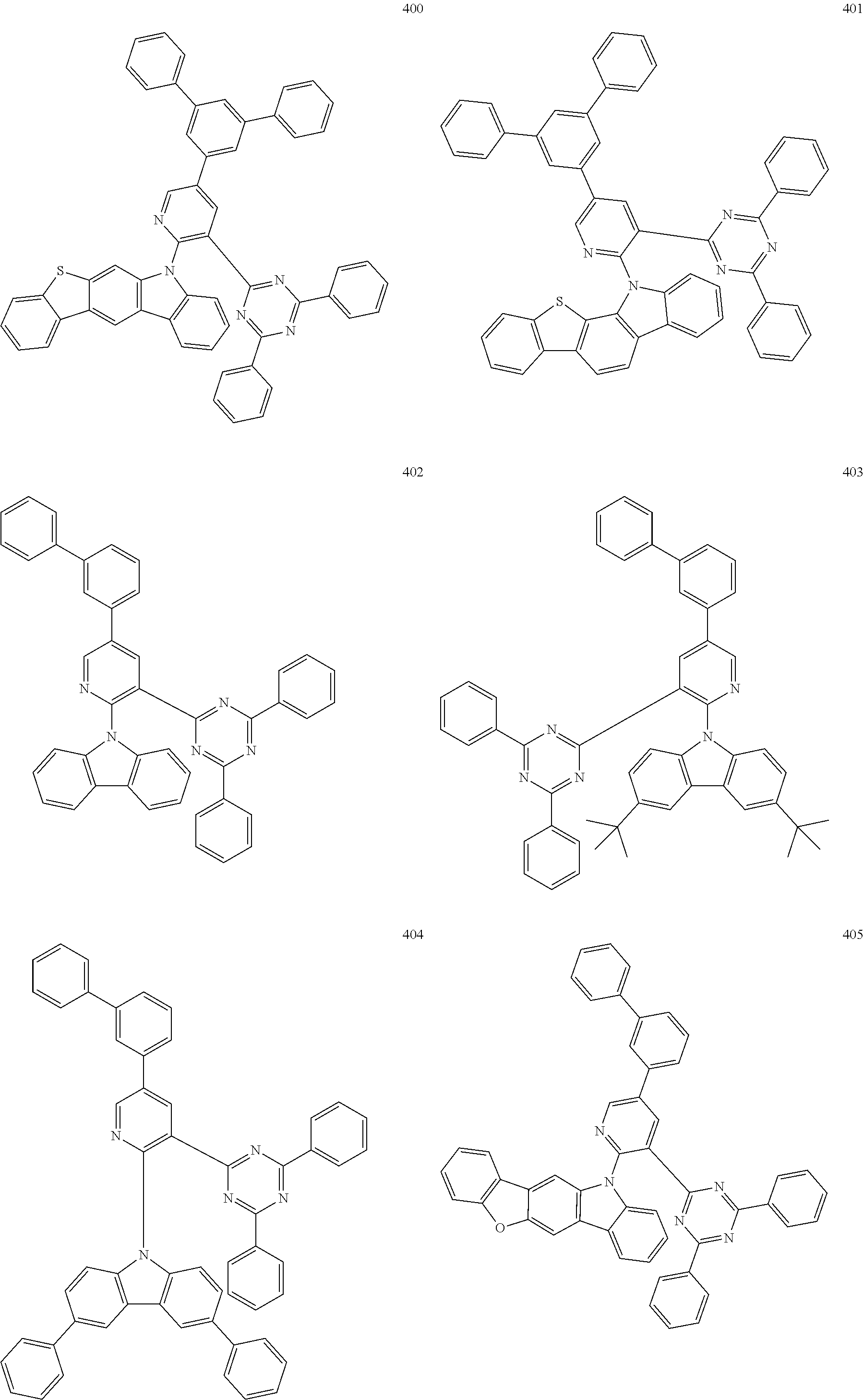

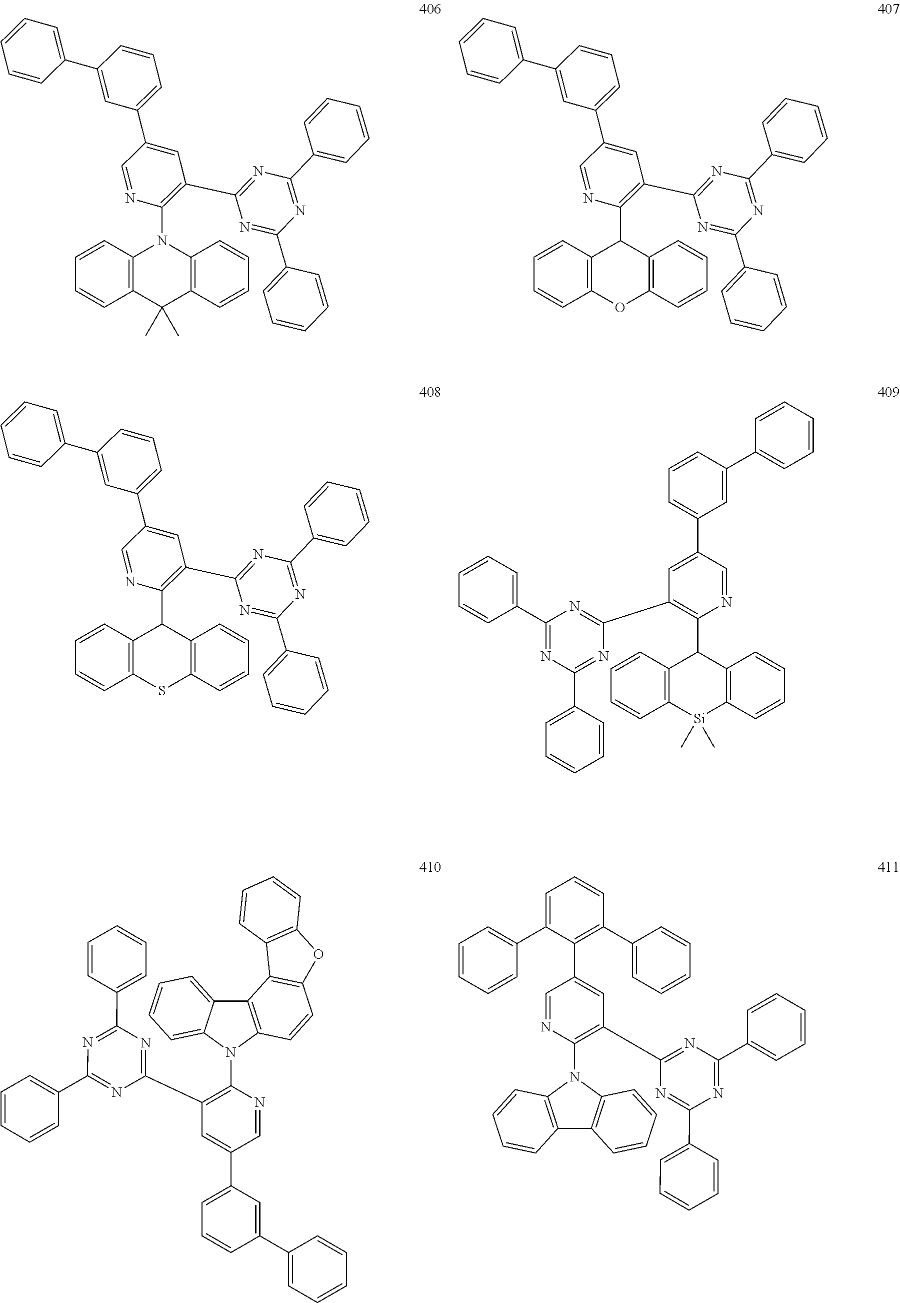

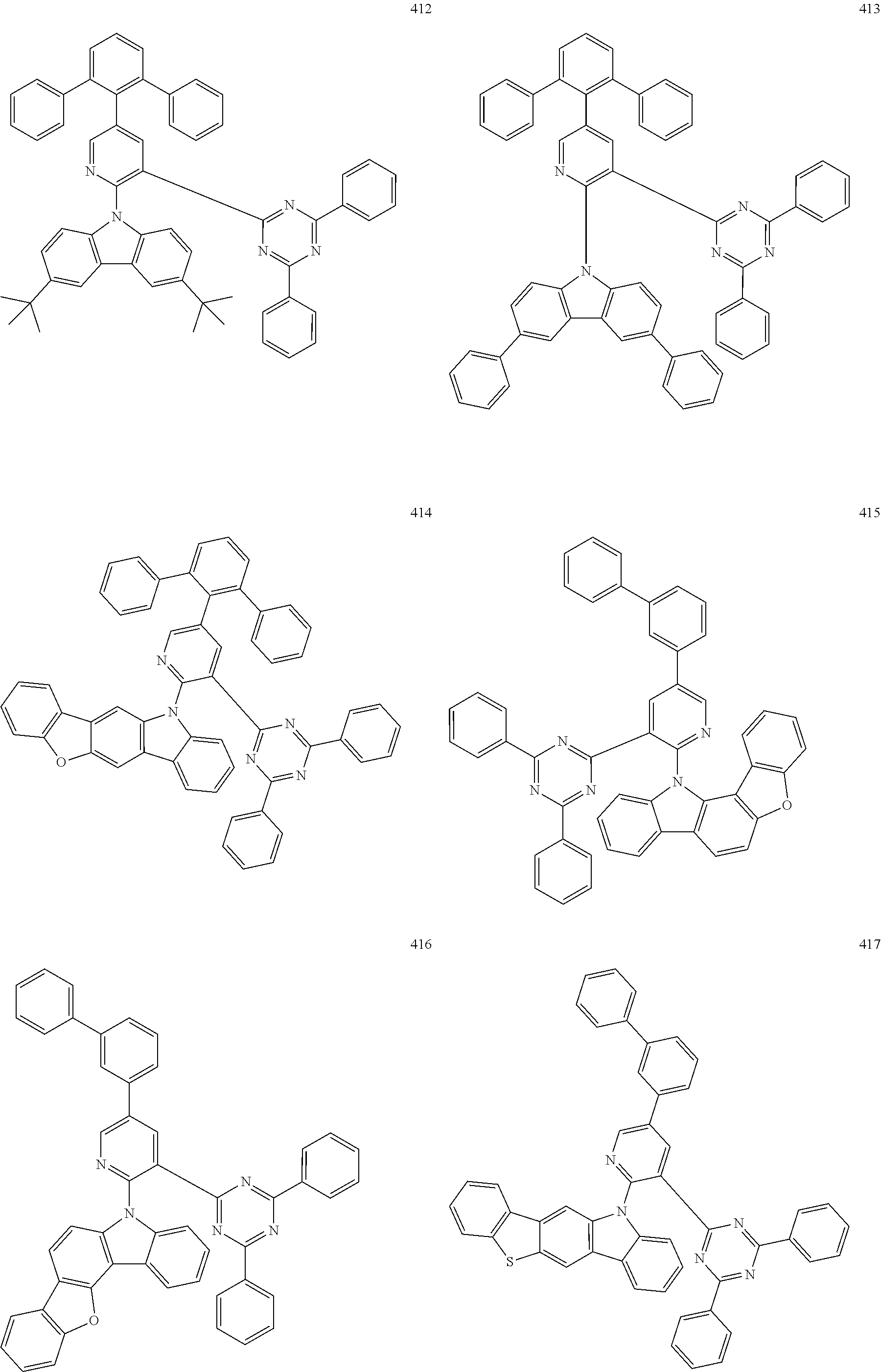

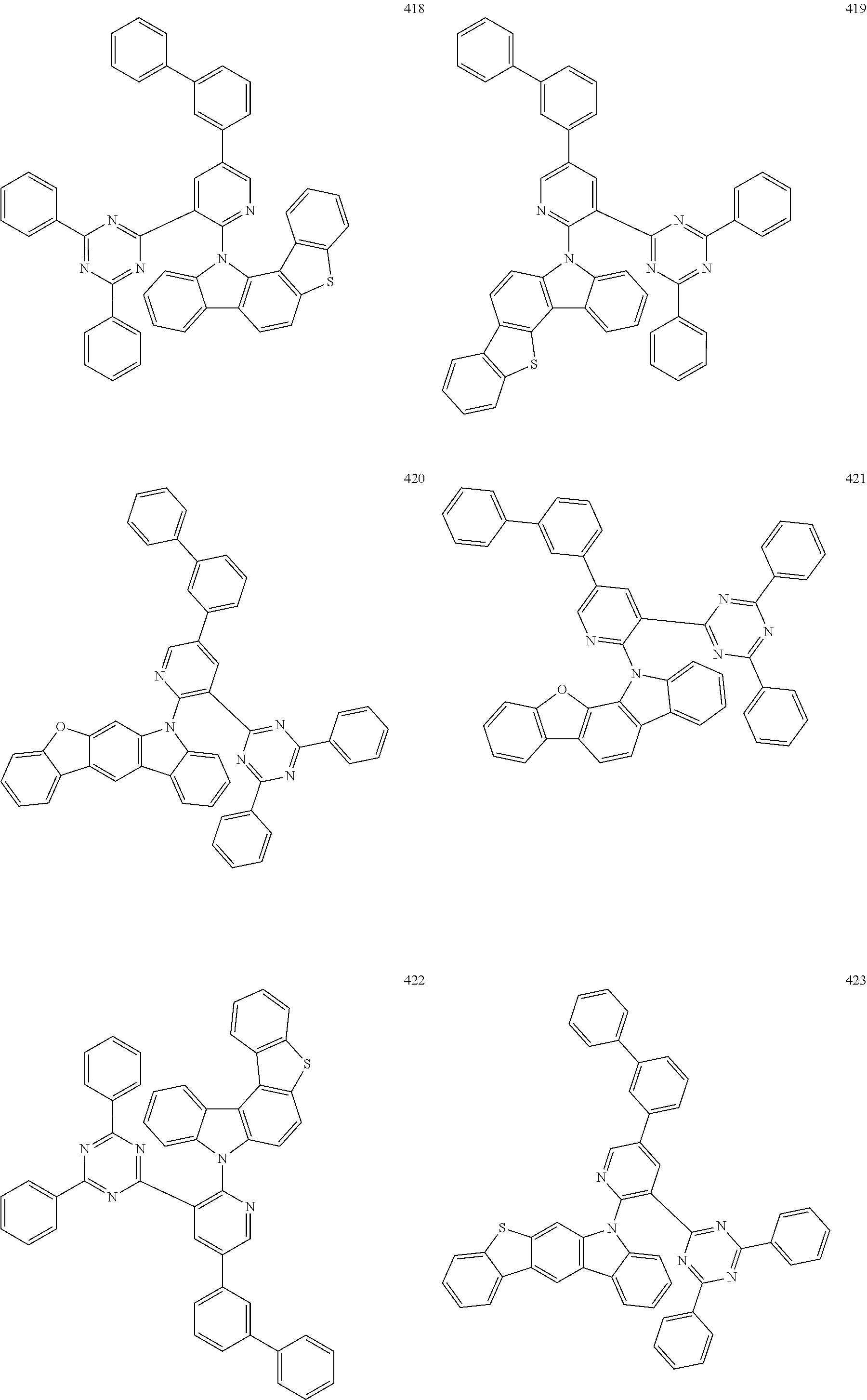

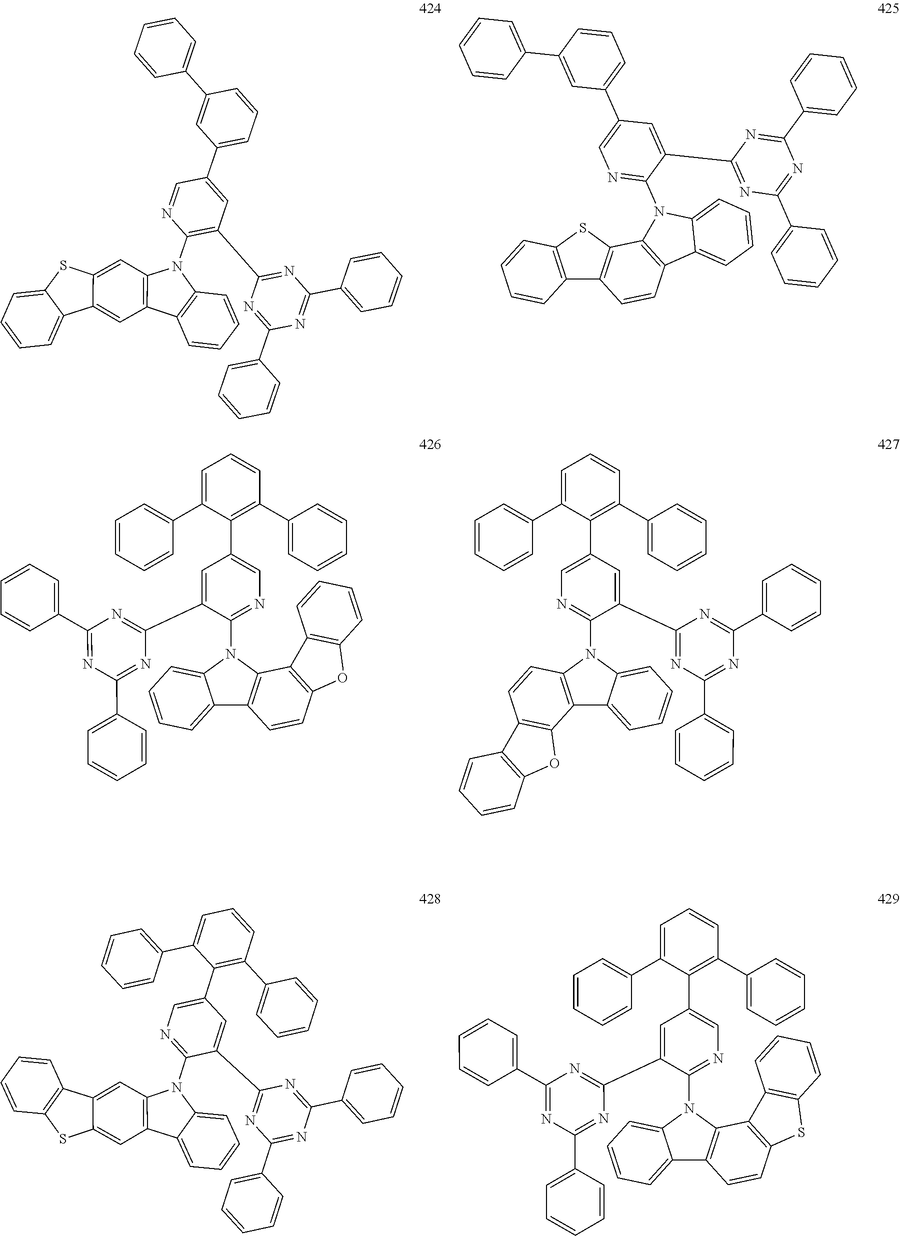

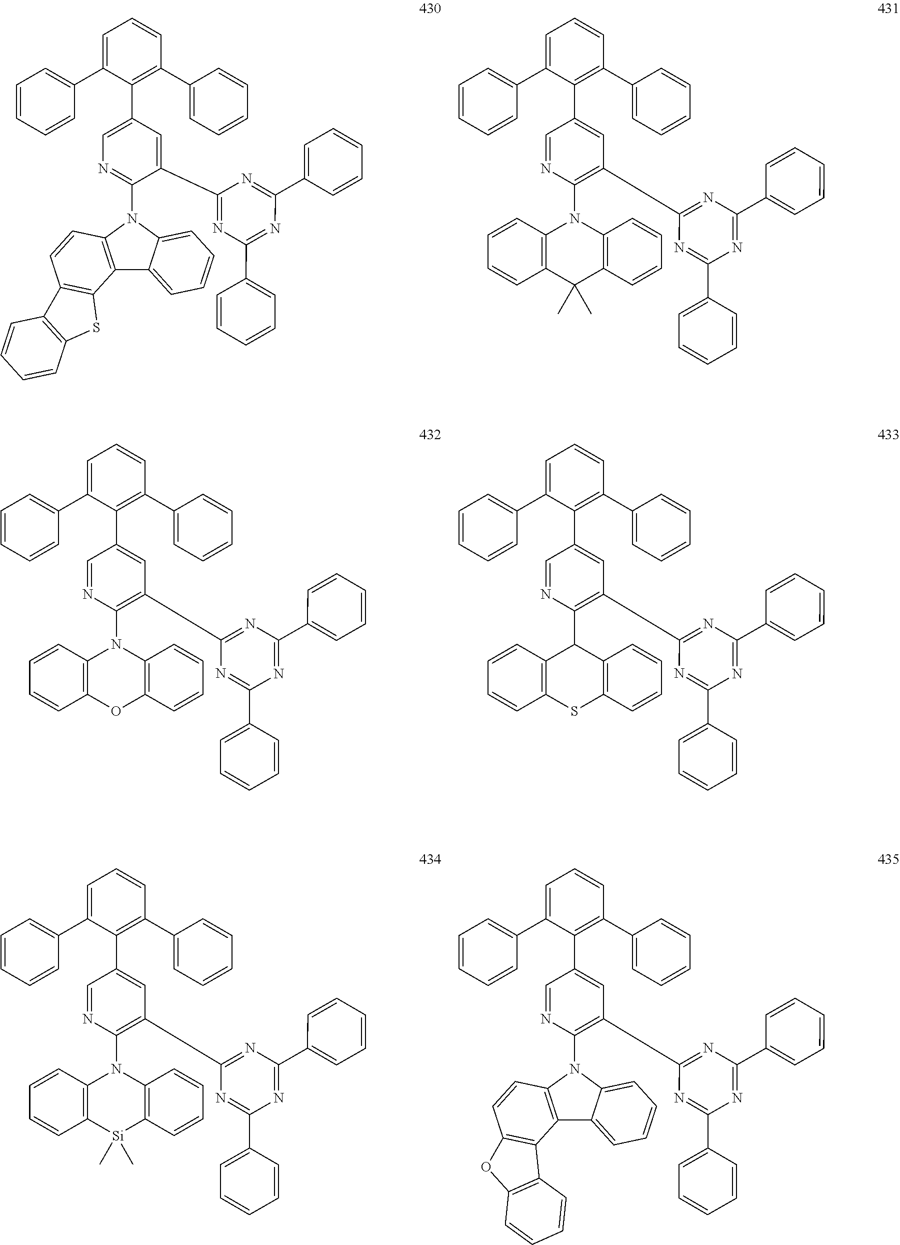

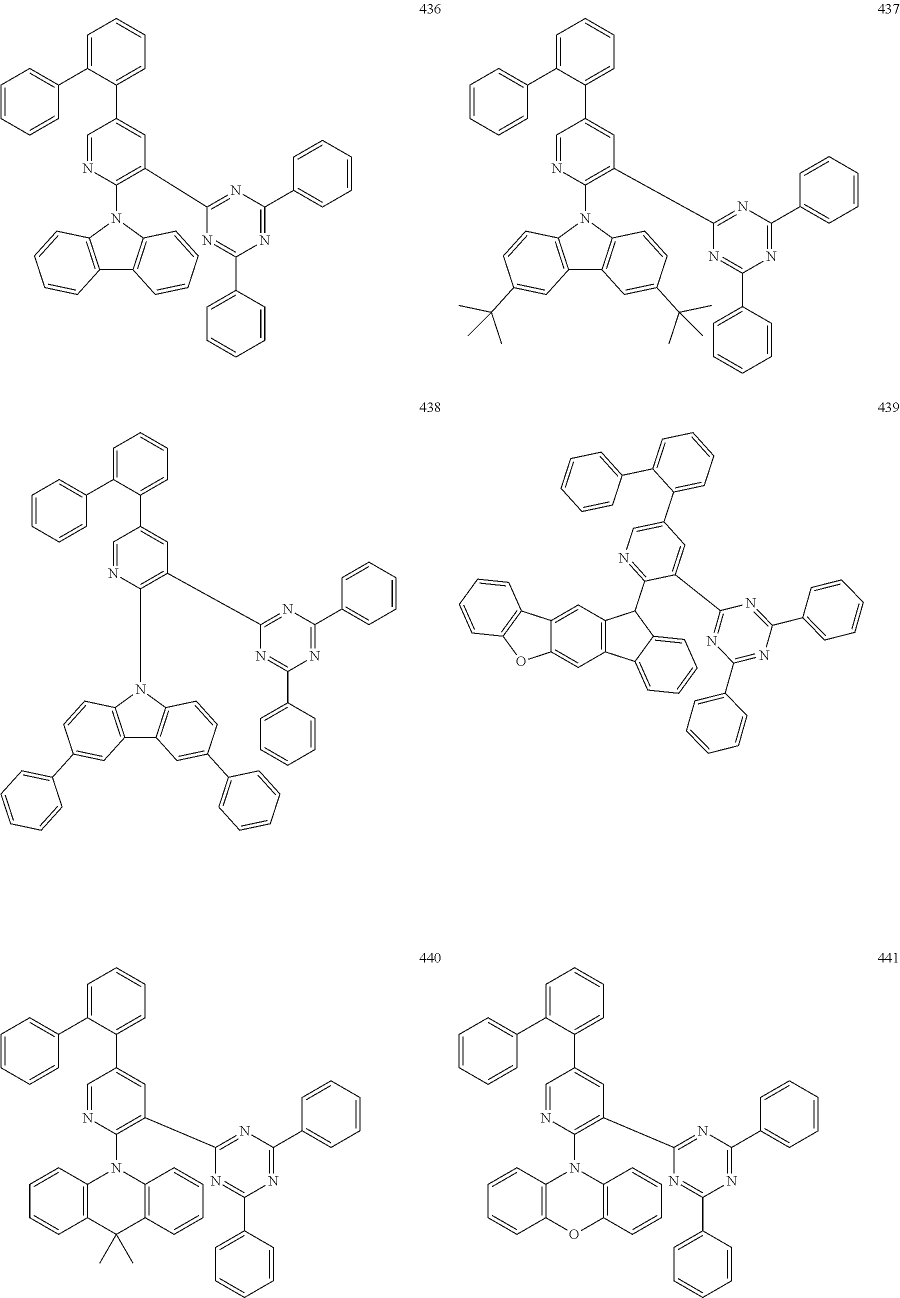

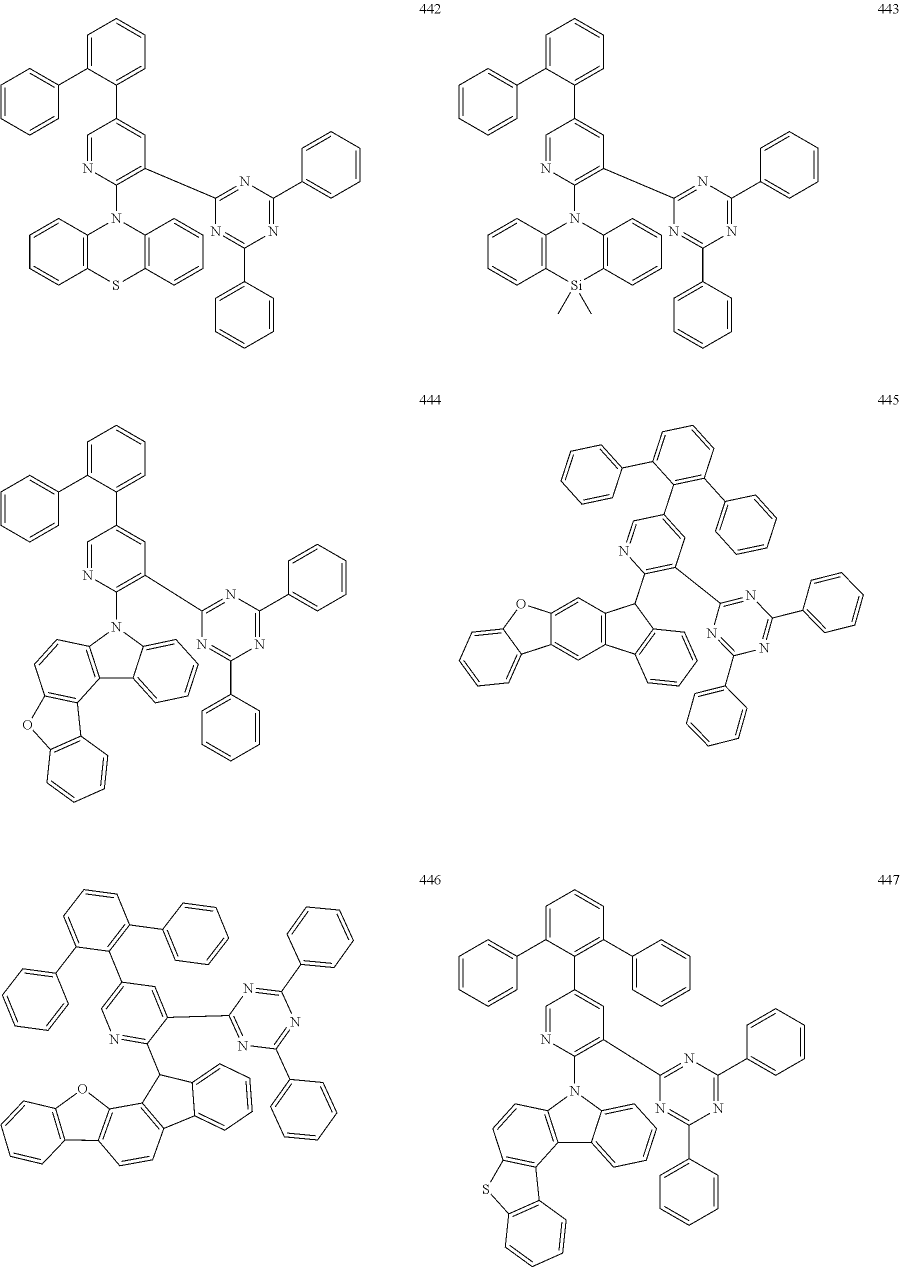

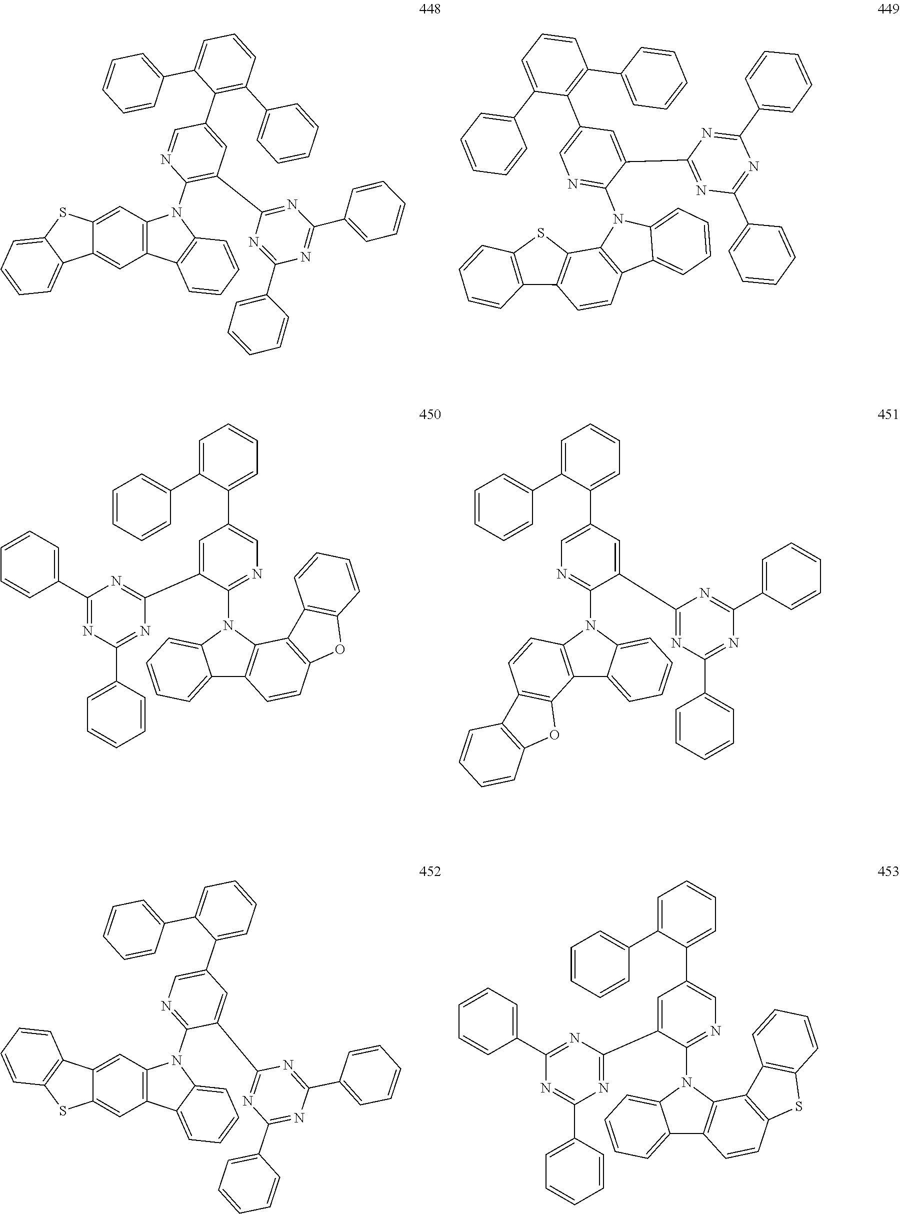

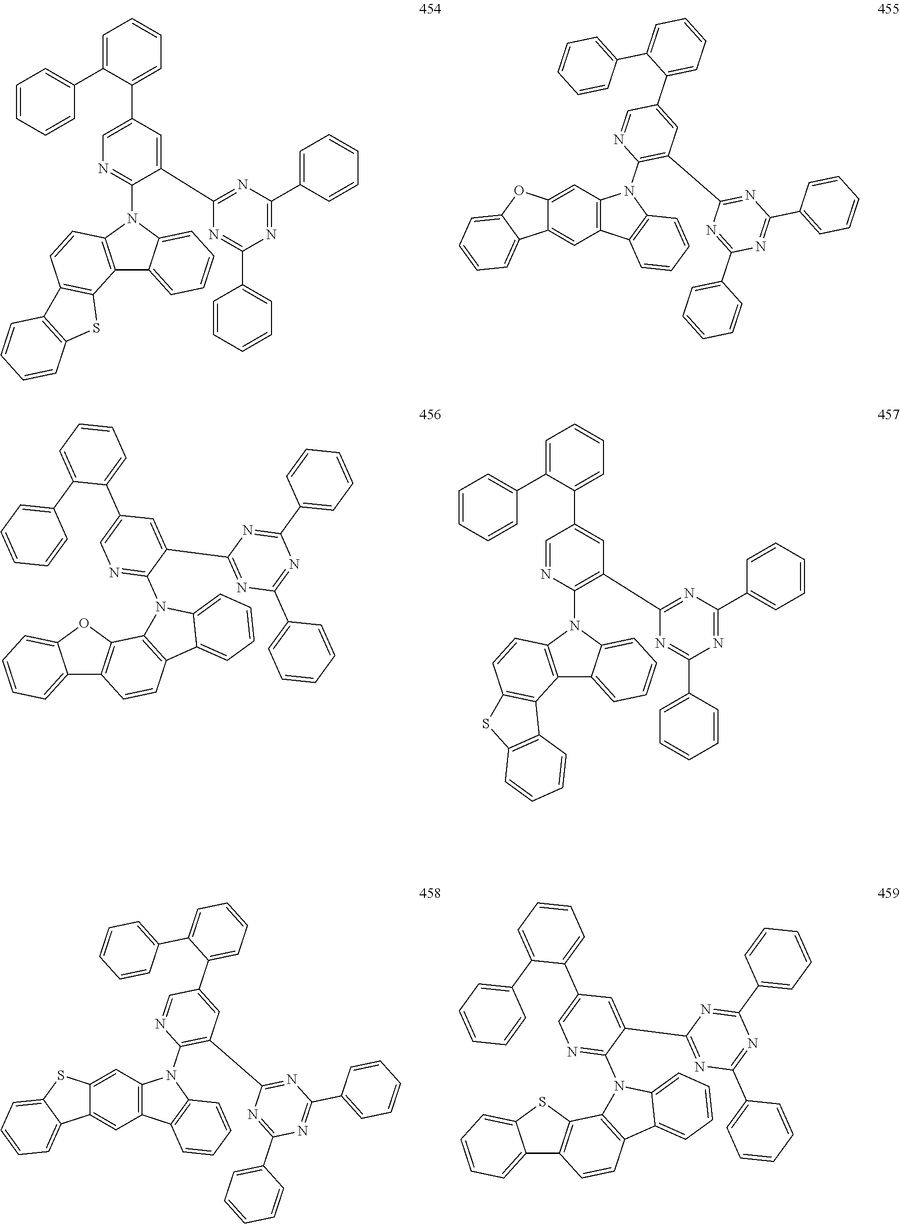

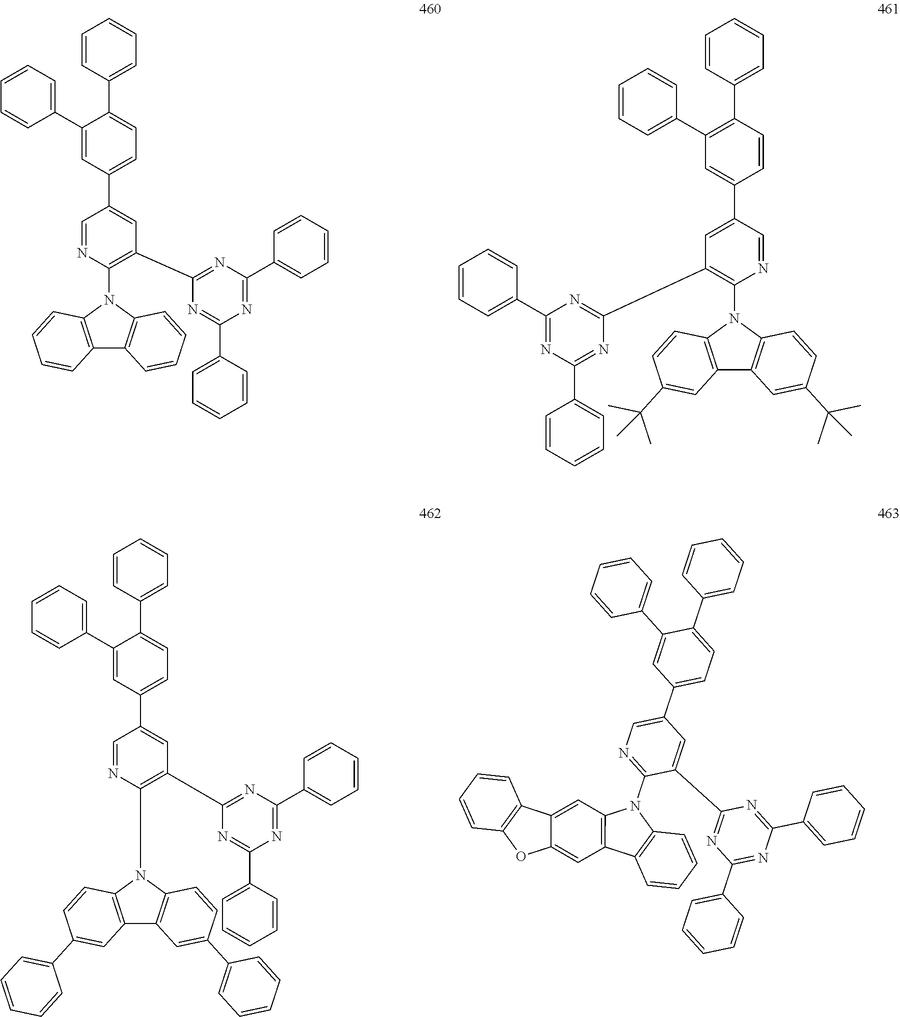 [SMILES: c1ccc(-c2nc(-c3ccccc3)nc(-c3cc(-c4ccc(-c5ccccc5)c(-c5ccccc5)c4)cnc3-n3c4ccccc4c4ccccc43)n2)cc1 ; CC(C)(C)c1ccc2c(c1)c1cc(C(C)(C)C)ccc1n2-c1ncc(-c2ccc(-c3ccccc3)c(-c3ccccc3)c2)cc1-c1nc(-c2ccccc2)nc(-c2ccccc2)n1 ; c1ccc(-c2ccc3c(c2)c2cc(-c4ccccc4)ccc2n3-c2ncc(-c3ccc(-c4ccccc4)c(-c4ccccc4)c3)cc2-c2nc(-c3ccccc3)nc(-c3ccccc3)n2)cc1 ; c1ccc(-c2nc(-c3ccccc3)nc(-c3cc(-c4ccc(-c5ccccc5)c(-c5ccccc5)c4)cnc3-n3c4ccccc4c4cc5oc6ccccc6c5cc43)n2)cc1]

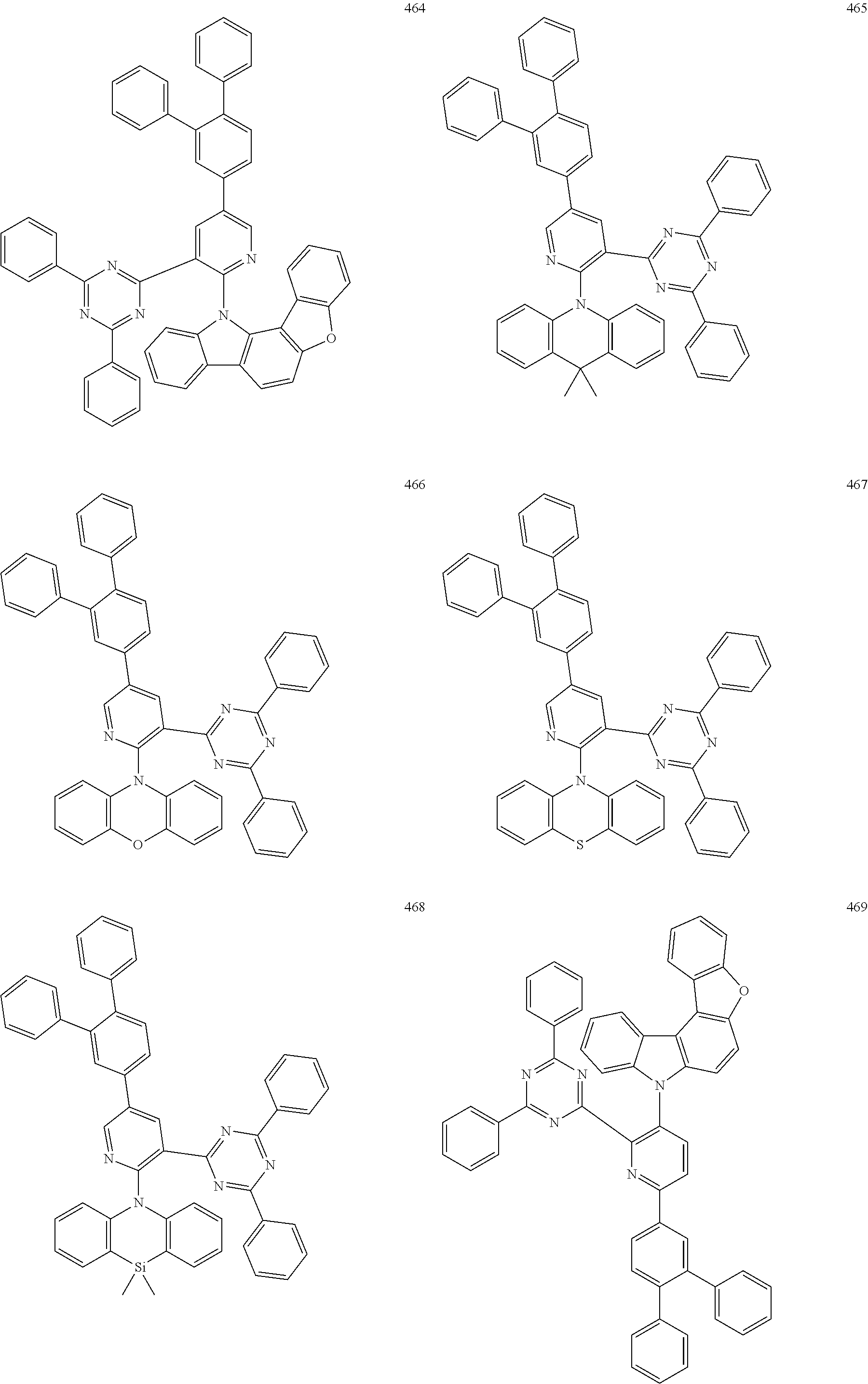

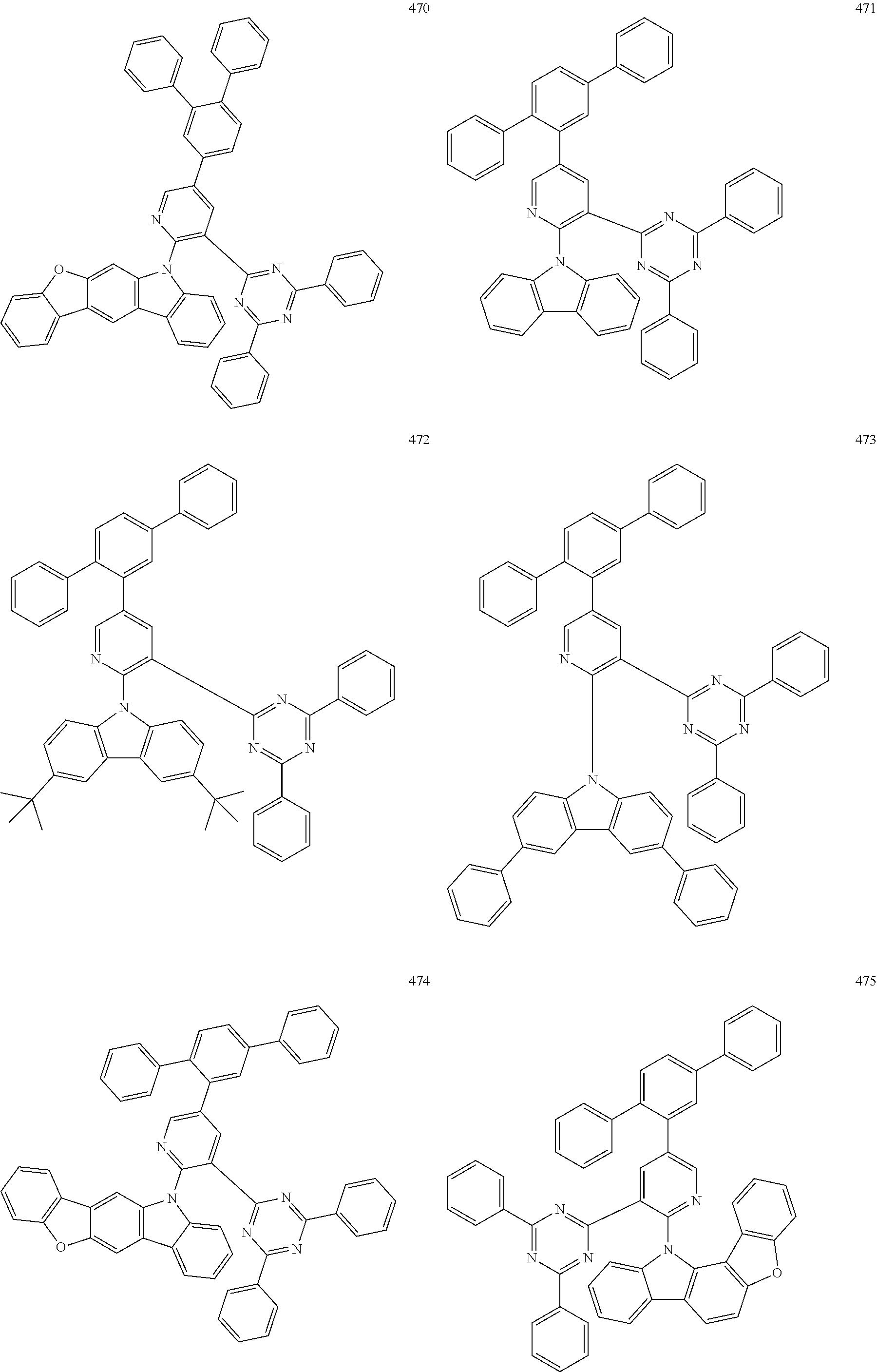

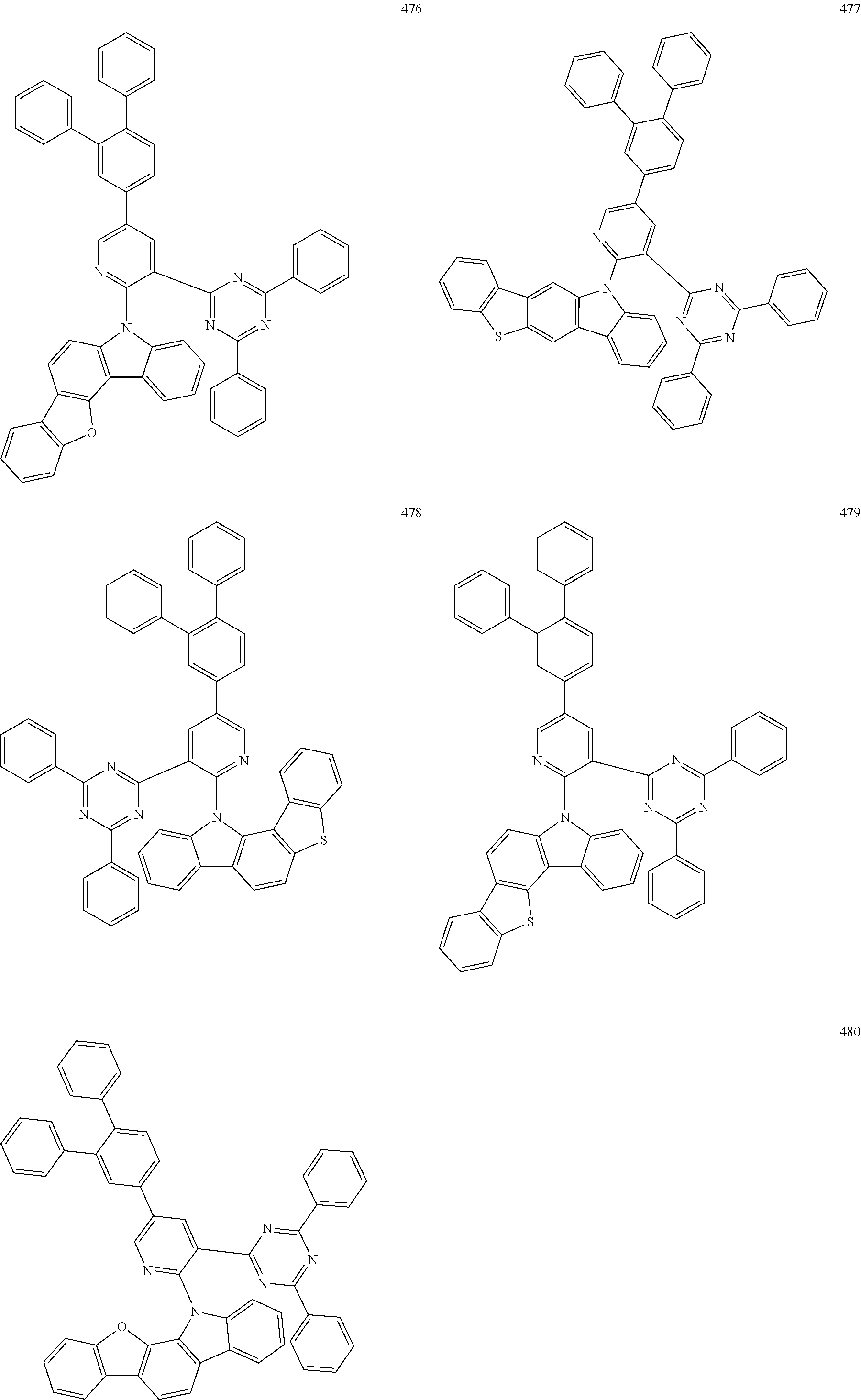

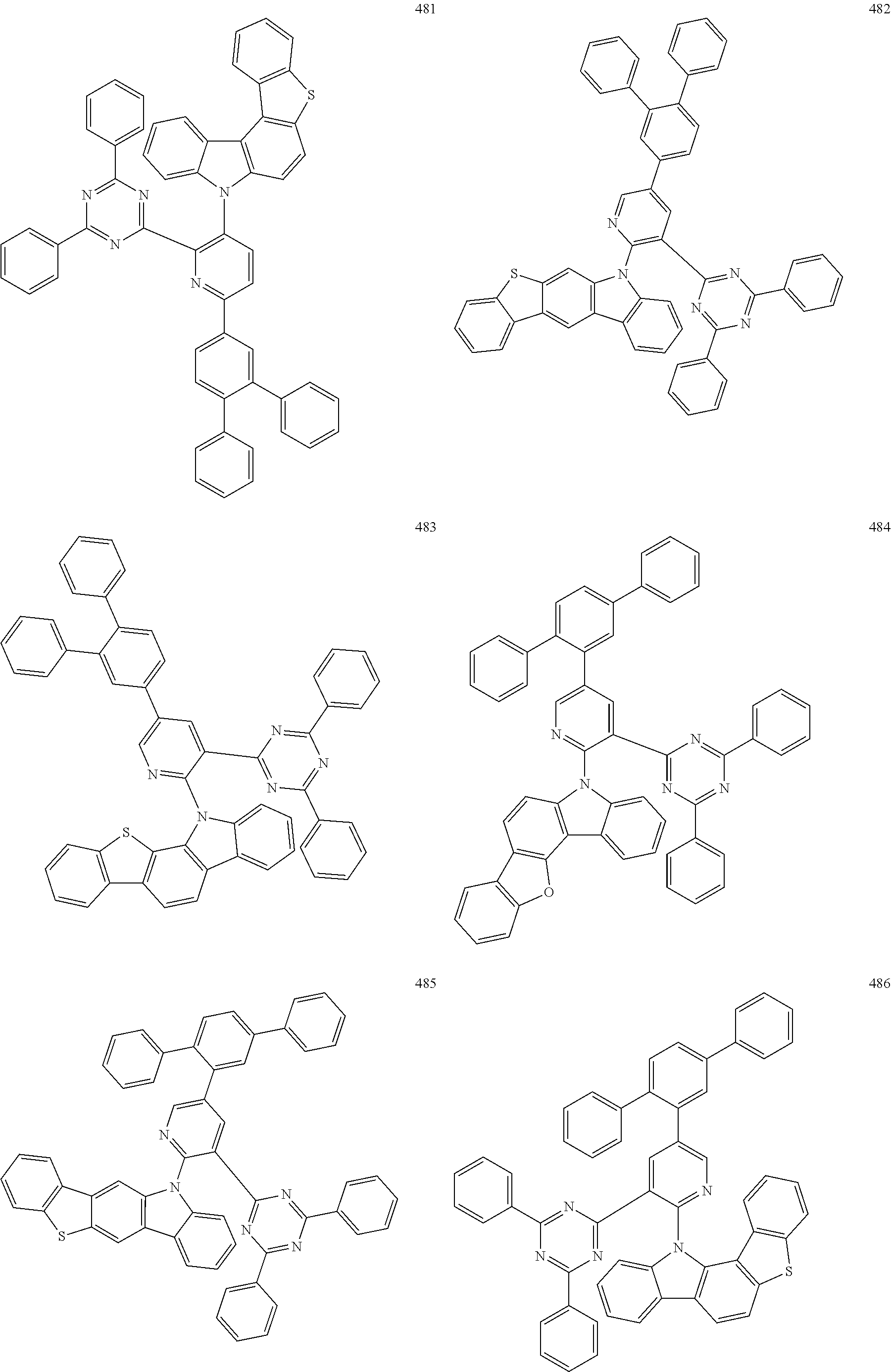

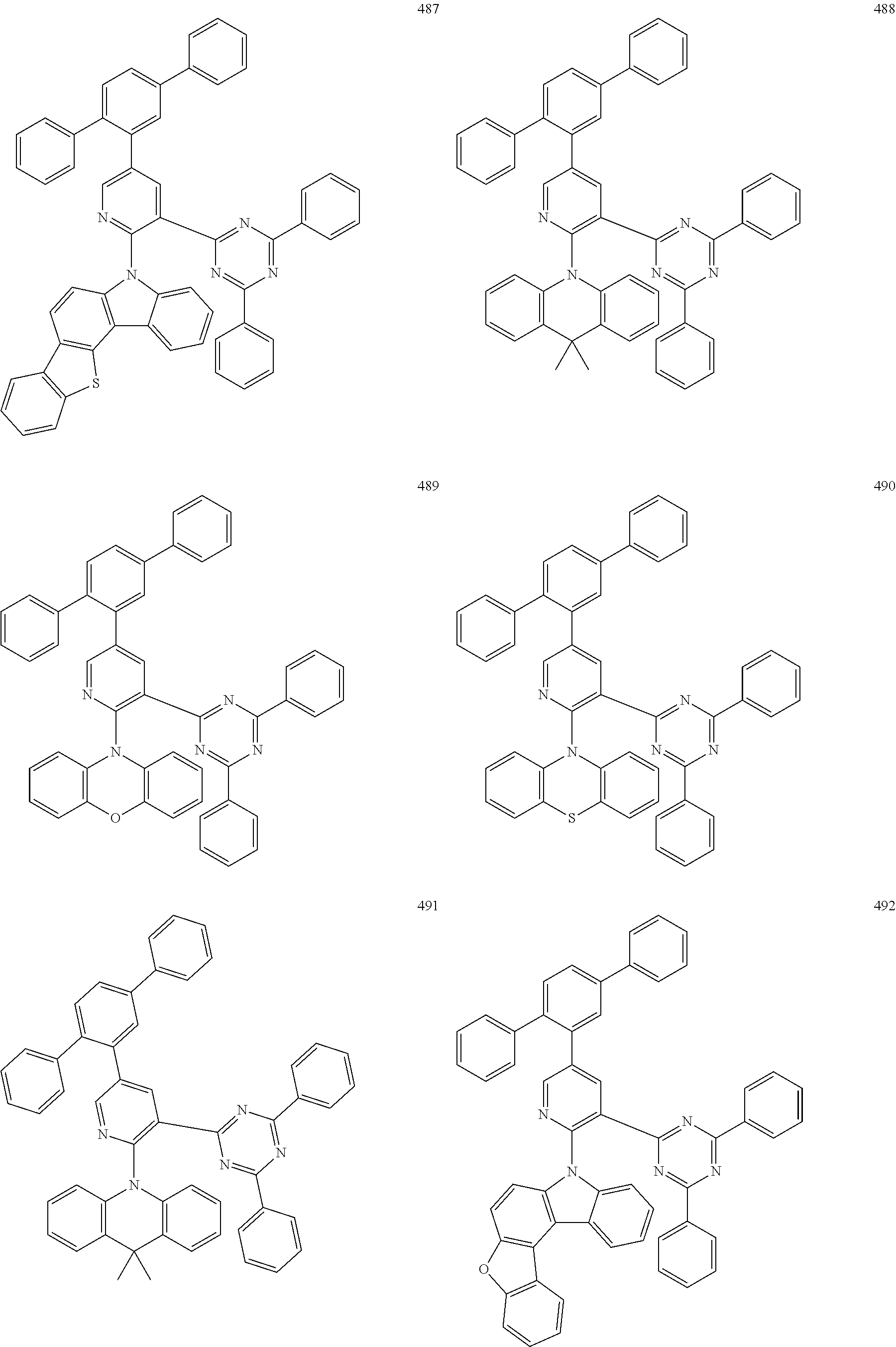

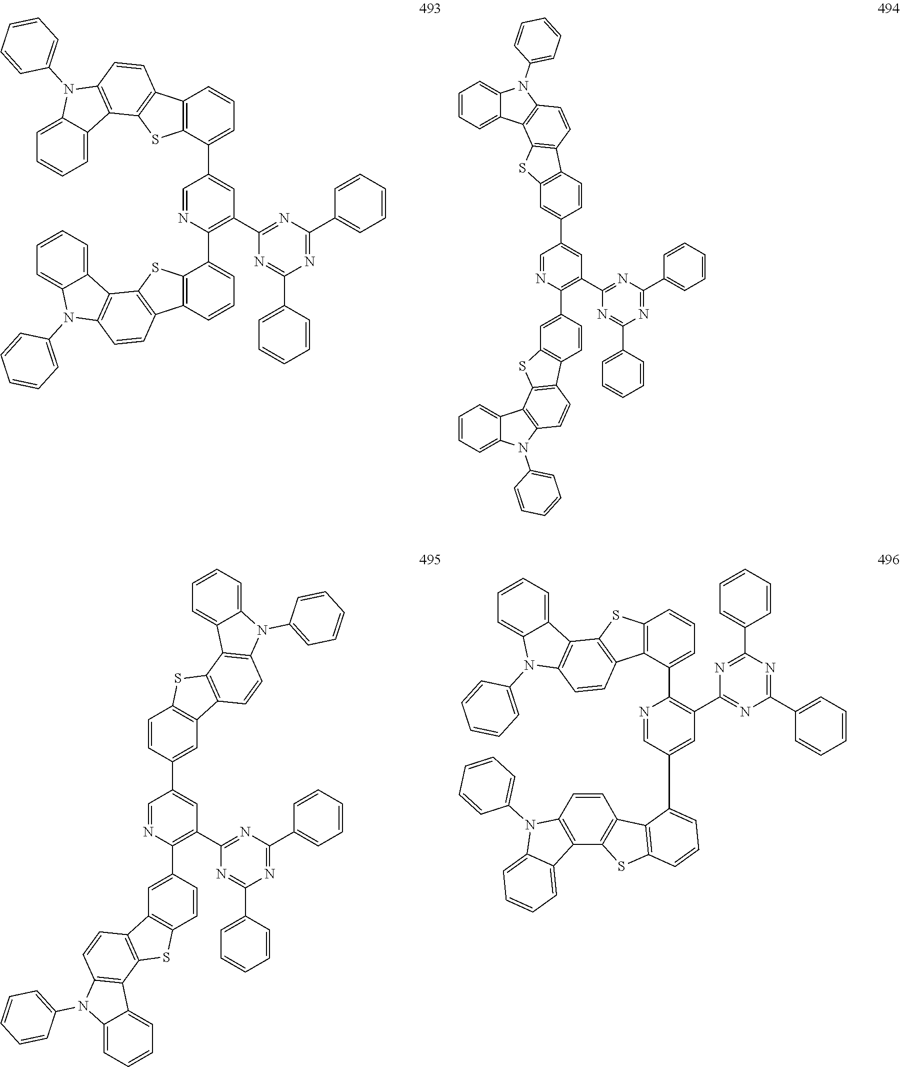

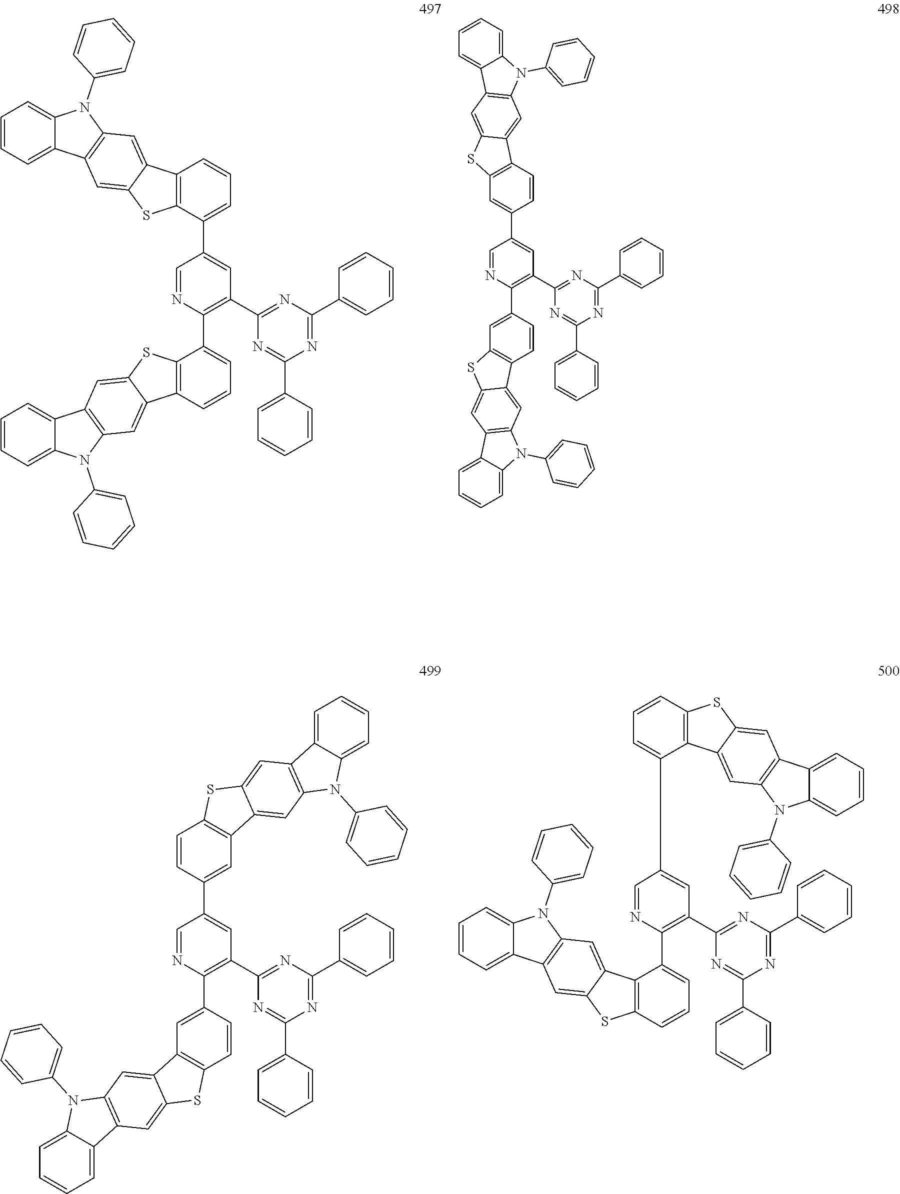

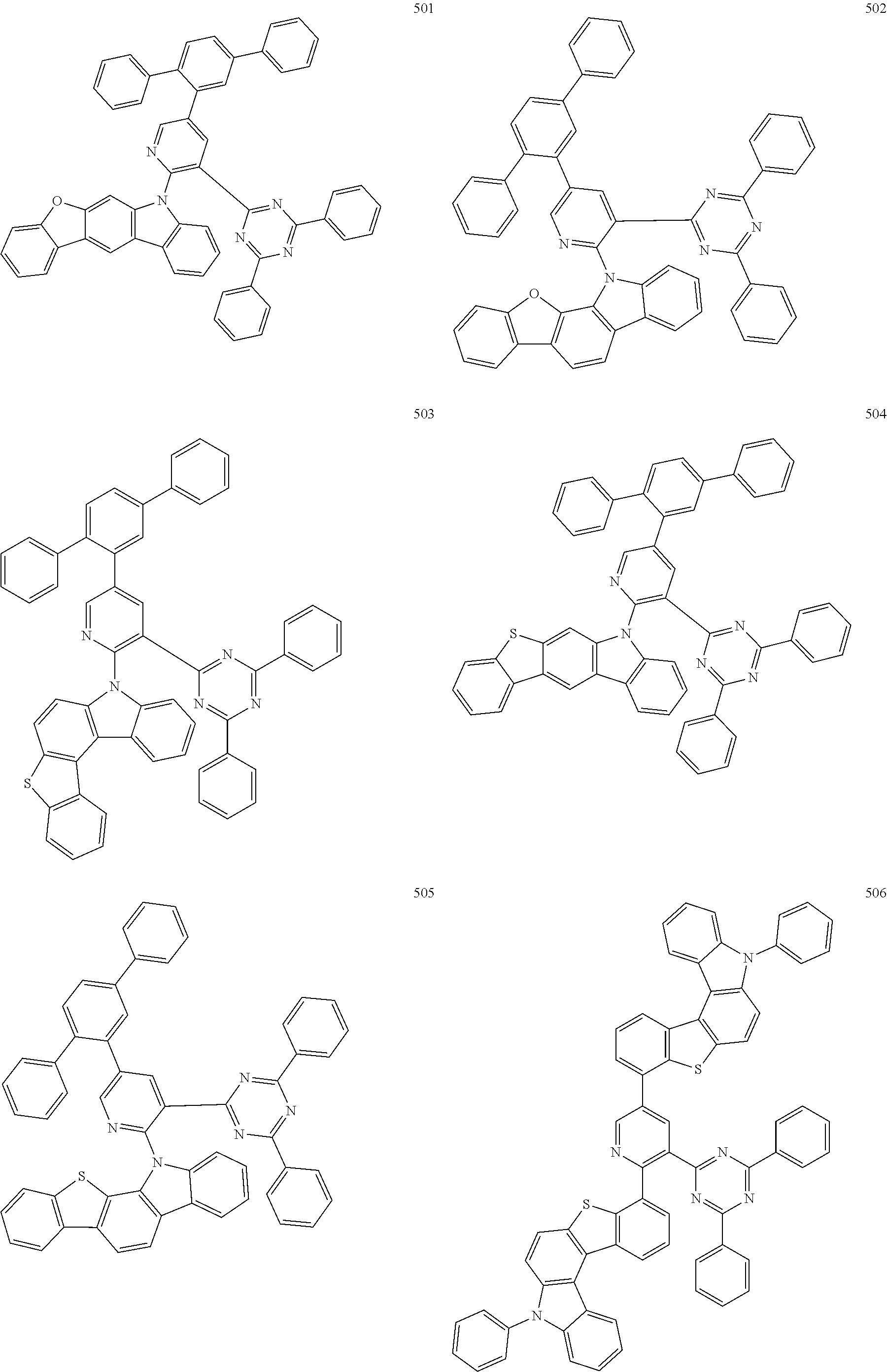

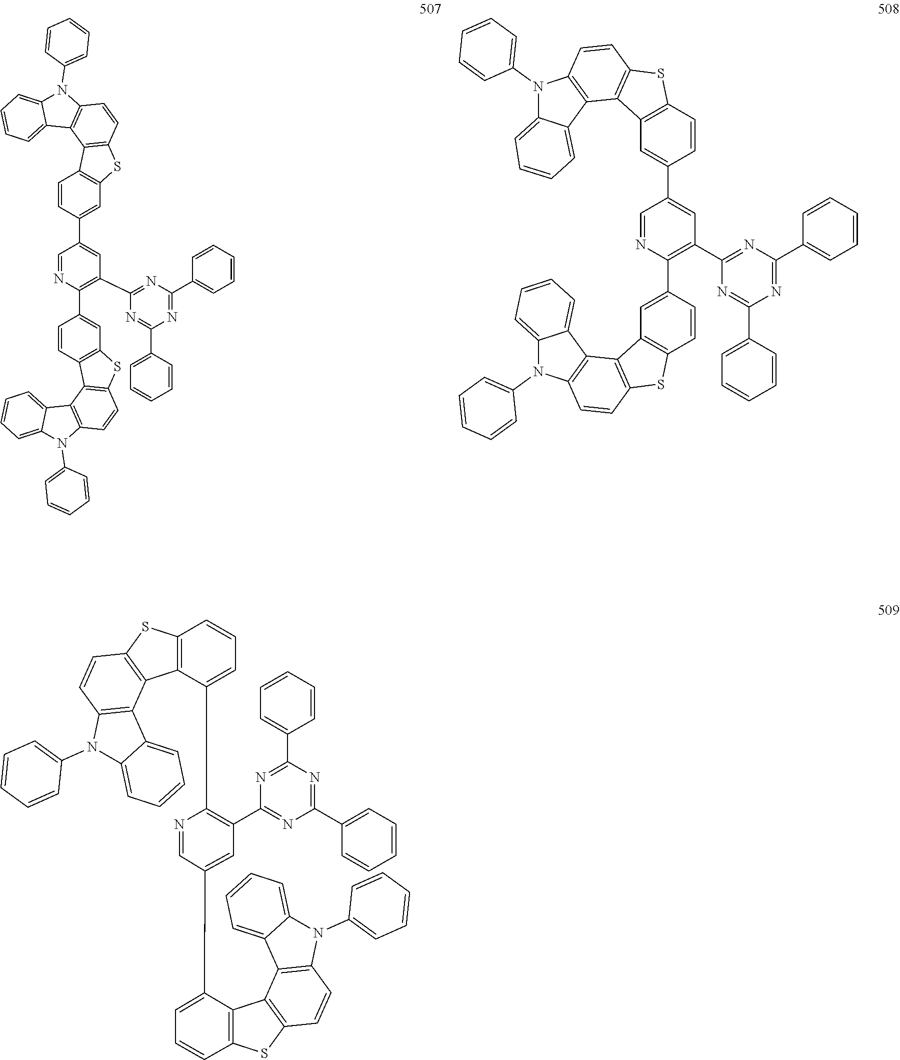 [SMILES: c1ccc(-c2nc(-c3ccccc3)nc(-c3cc(-c4ccc5c(c4)sc4ccc6c(c7ccccc7n6-c6ccccc6)c45)cnc3-c3ccc4c(c3)sc3ccc5c(c6ccccc6n5-c5ccccc5)c34)n2)cc1 ; c1ccc(-c2nc(-c3ccccc3)nc(-c3cc(-c4ccc5sc6ccc7c(c8ccccc8n7-c7ccccc7)c6c5c4)cnc3-c3ccc4sc5ccc6c(c7ccccc7n6-c6ccccc6)c5c4c3)n2)cc1 ; c1ccc(-c2nc(-c3ccccc3)nc(-c3cc(-c4cccc5sc6ccc7c(c8ccccc8n7-c7ccccc7)c6c45)cnc3-c3cccc4sc5ccc6c(c7ccccc7n6-c6ccccc6)c5c34)n2)cc1]

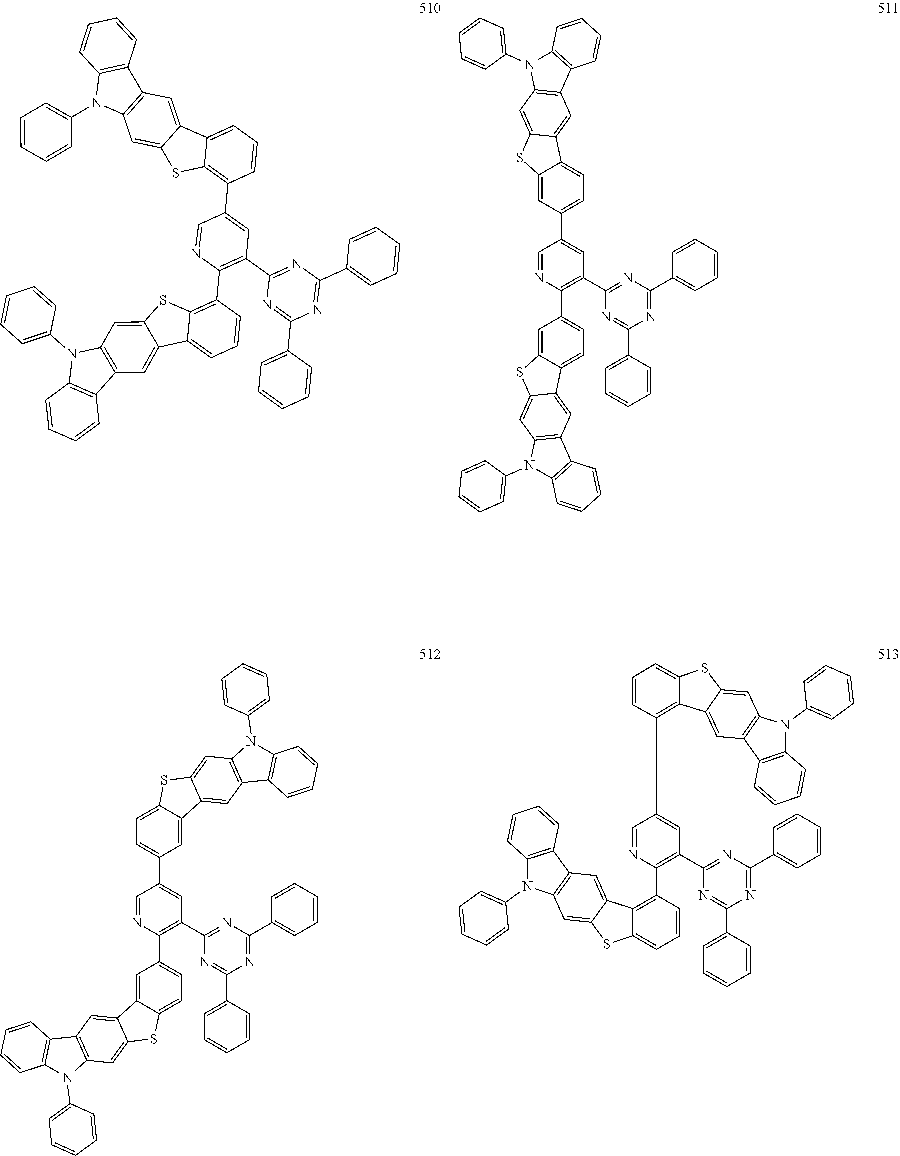

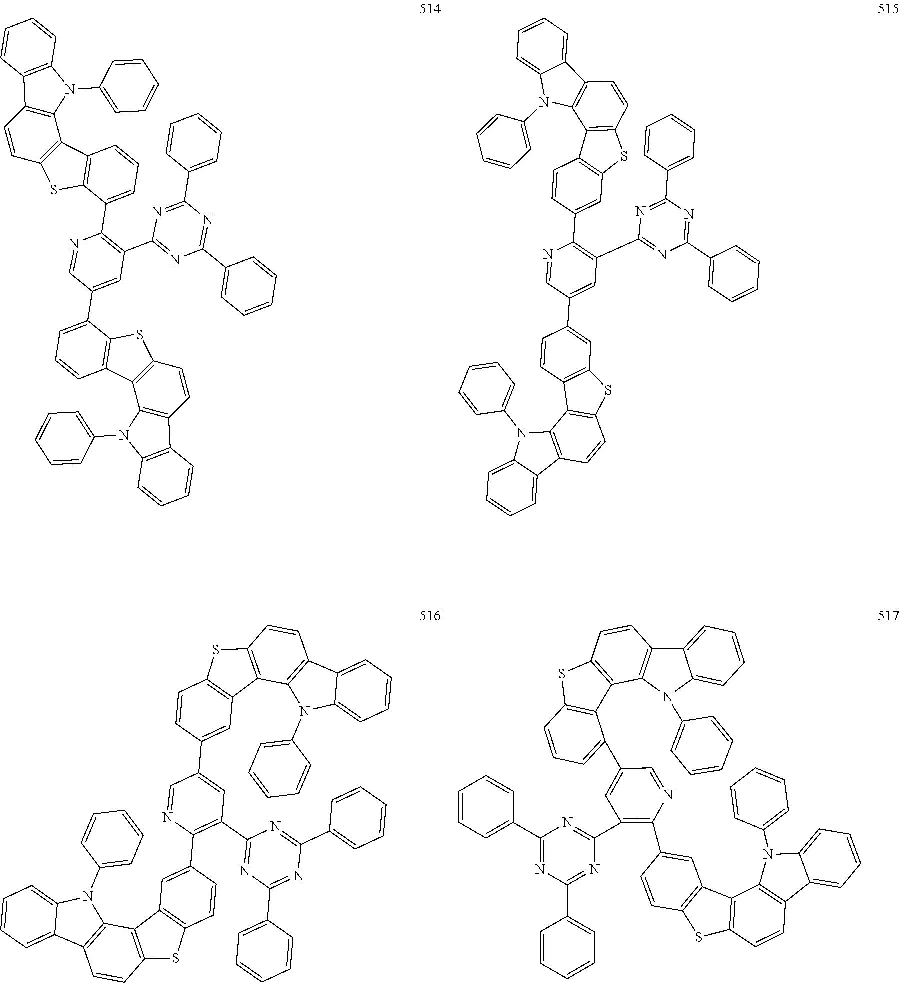

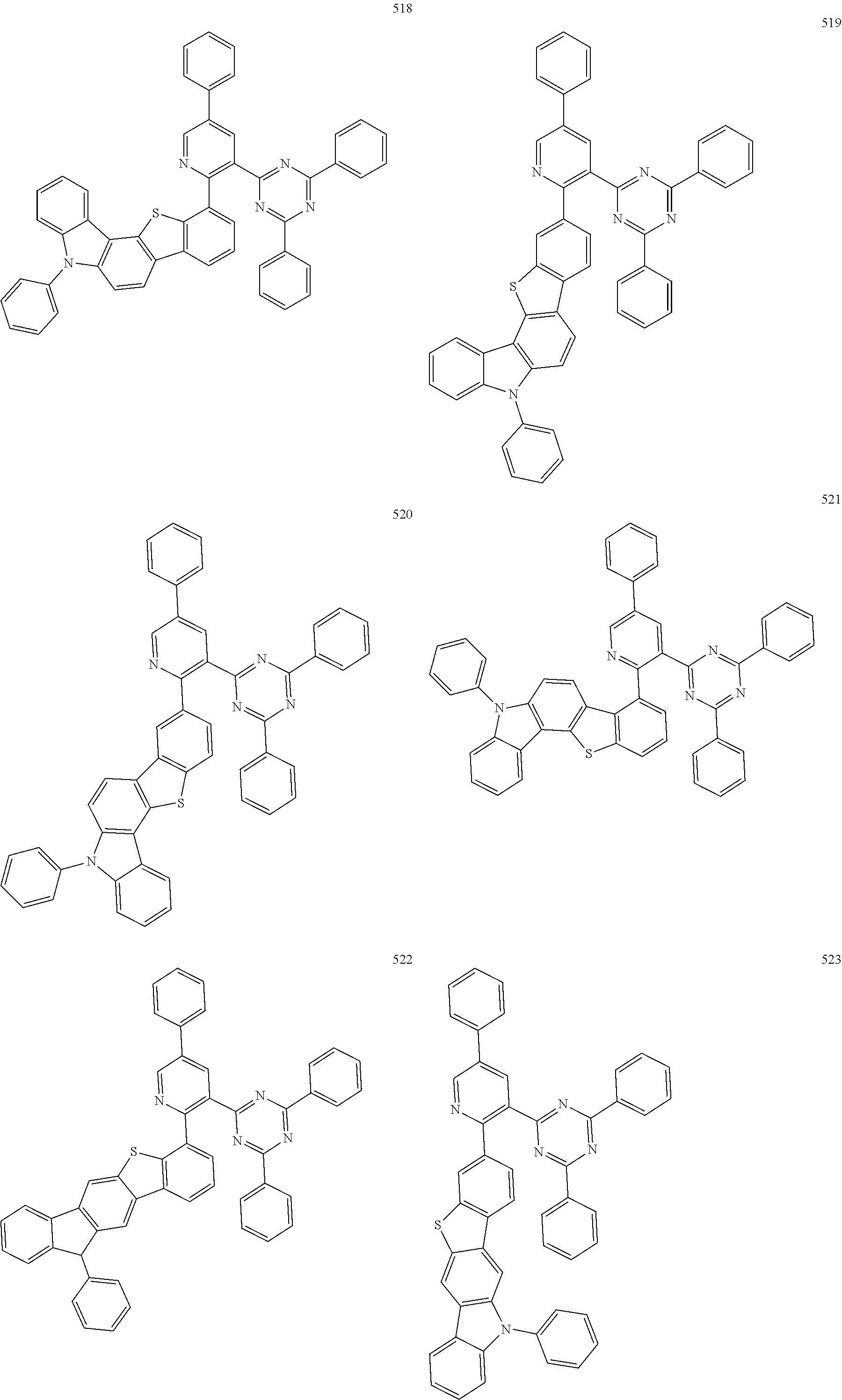

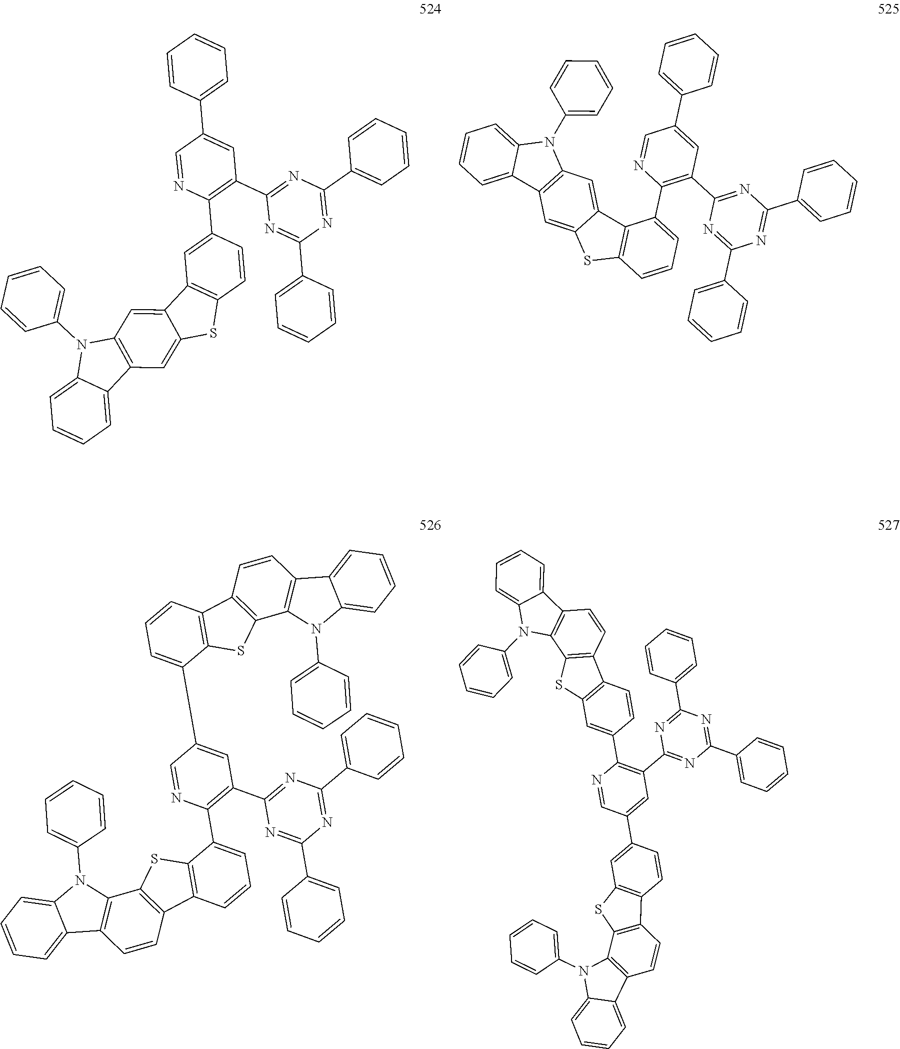 [SMILES: c1ccc(-c2cnc(-c3ccc4sc5cc6c7ccccc7n(-c7ccccc7)c6cc5c4c3)c(-c3nc(-c4ccccc4)nc(-c4ccccc4)n3)c2)cc1 ; c1ccc(-c2cnc(-c3cccc4sc5cc6c7ccccc7n(-c7ccccc7)c6cc5c34)c(-c3nc(-c4ccccc4)nc(-c4ccccc4)n3)c2)cc1 ; c1ccc(-c2nc(-c3ccccc3)nc(-c3cc(-c4cccc5c4sc4c5ccc5c6ccccc6n(-c6ccccc6)c54)cnc3-c3cccc4c3sc3c4ccc4c5ccccc5n(-c5ccccc5)c43)n2)cc1 ; c1ccc(-c2nc(-c3ccccc3)nc(-c3cc(-c4ccc5c(c4)sc4c5ccc5c6ccccc6n(-c6ccccc6)c54)cnc3-c3ccc4c(c3)sc3c4ccc4c5ccccc5n(-c5ccccc5)c43)n2)cc1]

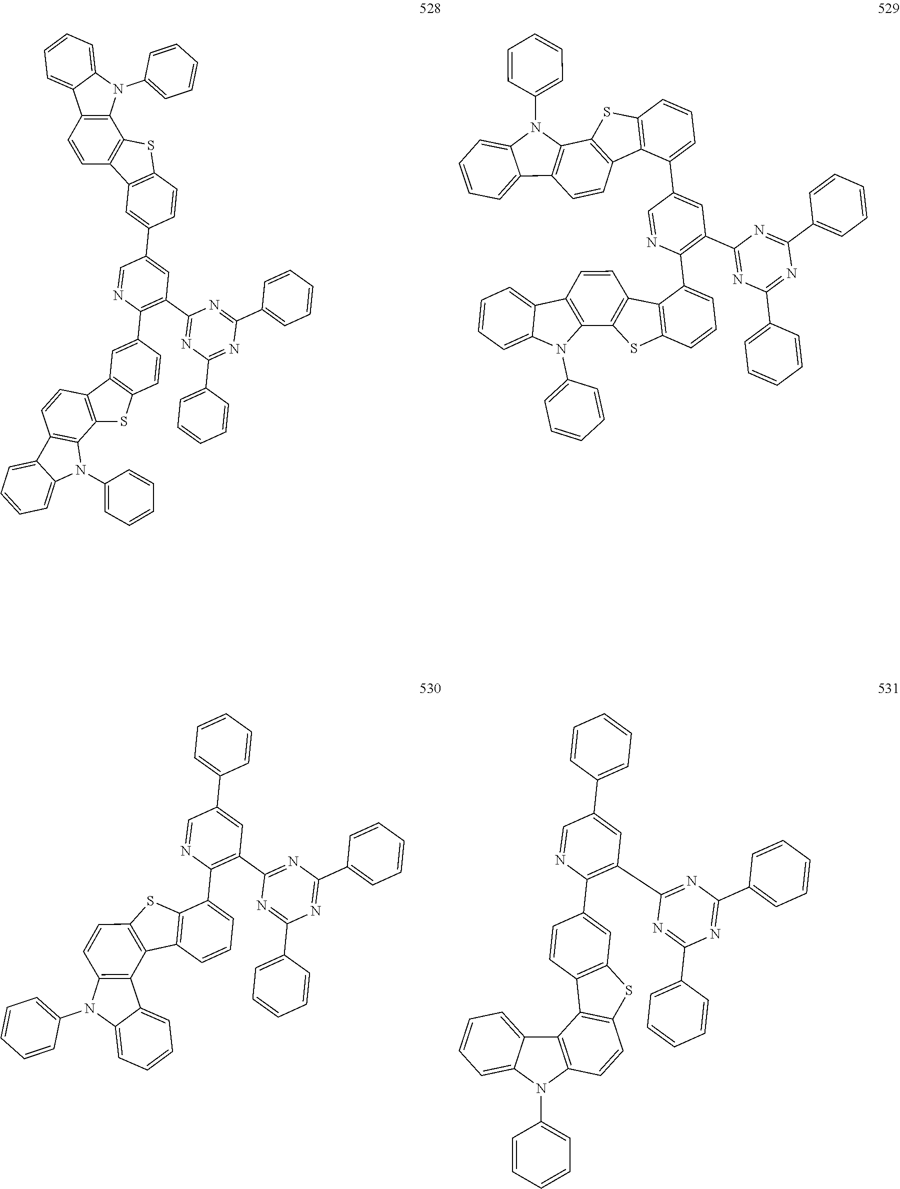 [SMILES: c1ccc(-c2nc(-c3ccccc3)nc(-c3cc(-c4ccc5sc6c(ccc7c8ccccc8n(-c8ccccc8)c76)c5c4)cnc3-c3ccc4sc5c(ccc6c7ccccc7n(-c7ccccc7)c65)c4c3)n2)cc1 ; c1ccc(-c2nc(-c3ccccc3)nc(-c3cc(-c4cccc5sc6c(ccc7c8ccccc8n(-c8ccccc8)c76)c45)cnc3-c3cccc4sc5c(ccc6c7ccccc7n(-c7ccccc7)c65)c34)n2)cc1 ; c1ccc(-c2cnc(-c3cccc4c3sc3ccc5c(c6ccccc6n5-c5ccccc5)c34)c(-c3nc(-c4ccccc4)nc(-c4ccccc4)n3)c2)cc1 ; c1ccc(-c2cnc(-c3ccc4c(c3)sc3ccc5c(c6ccccc6n5-c5ccccc5)c34)c(-c3nc(-c4ccccc4)nc(-c4ccccc4)n3)c2)cc1]

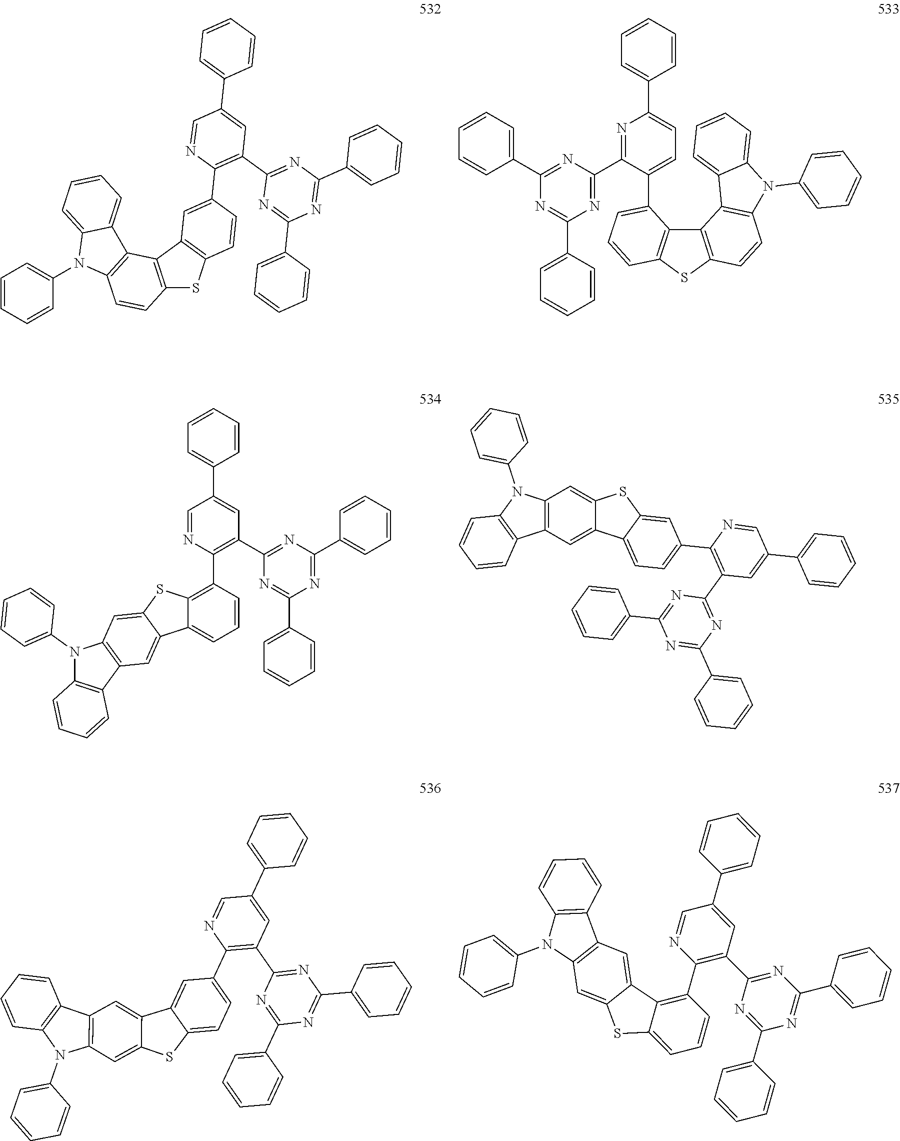

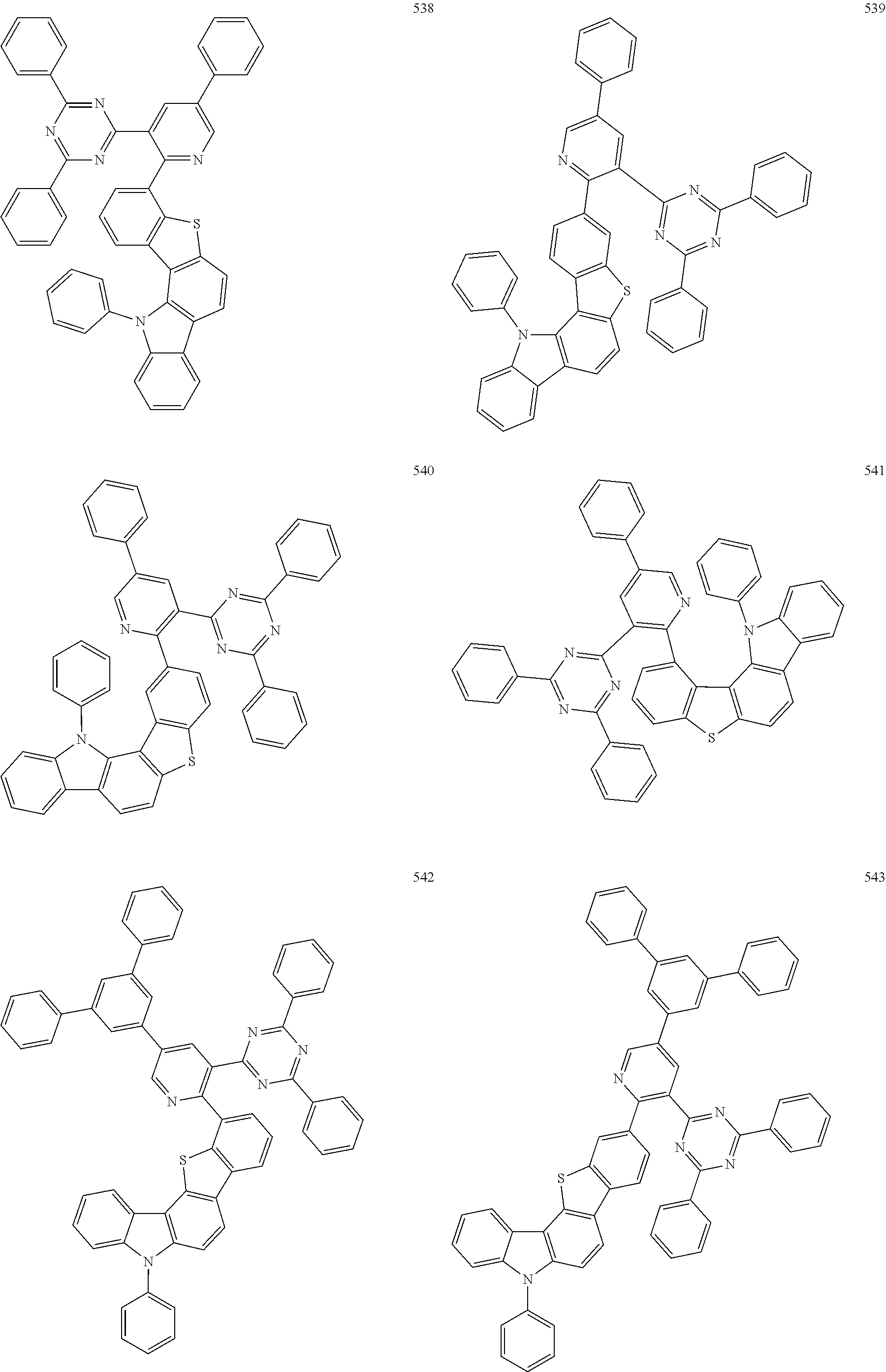

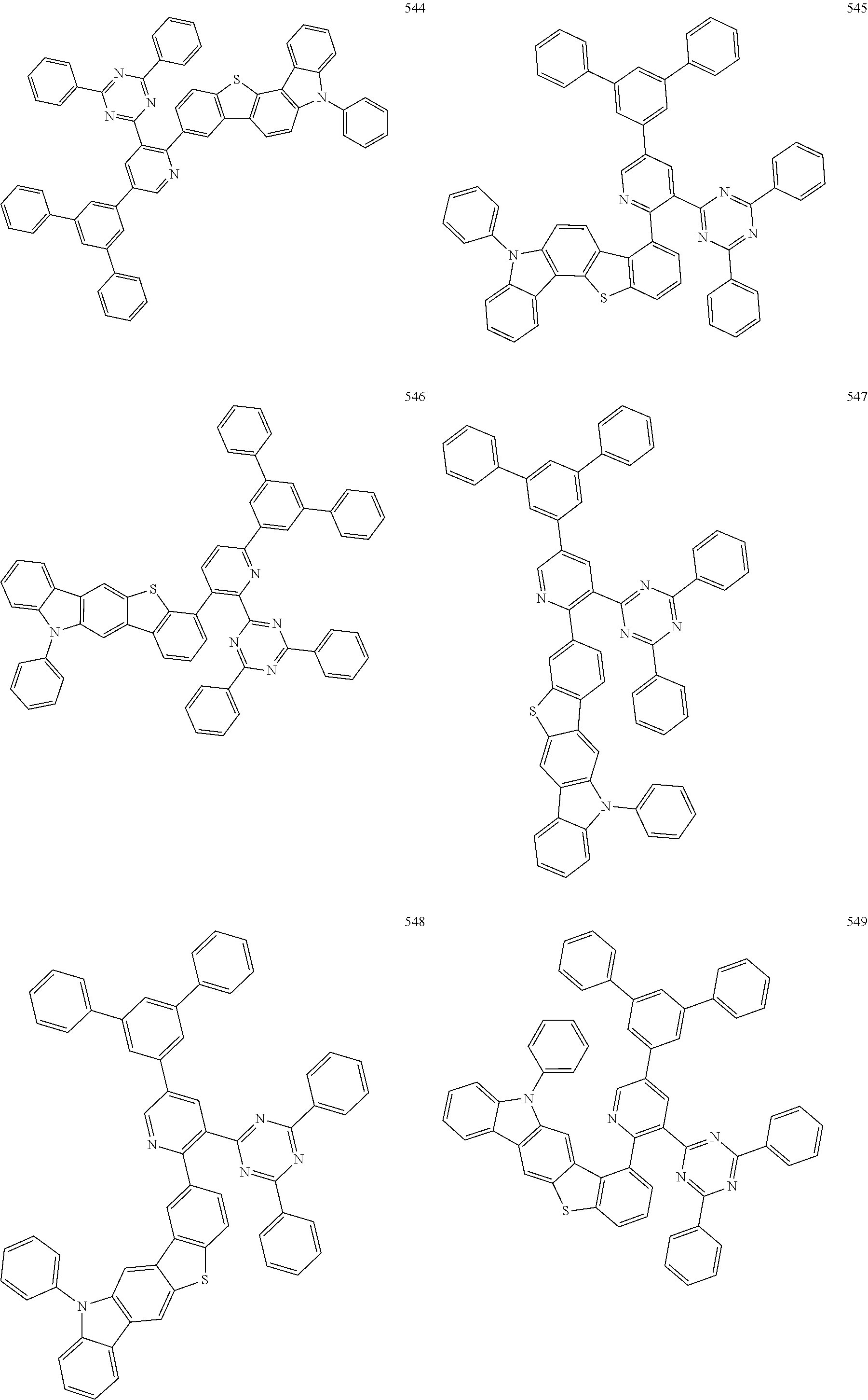

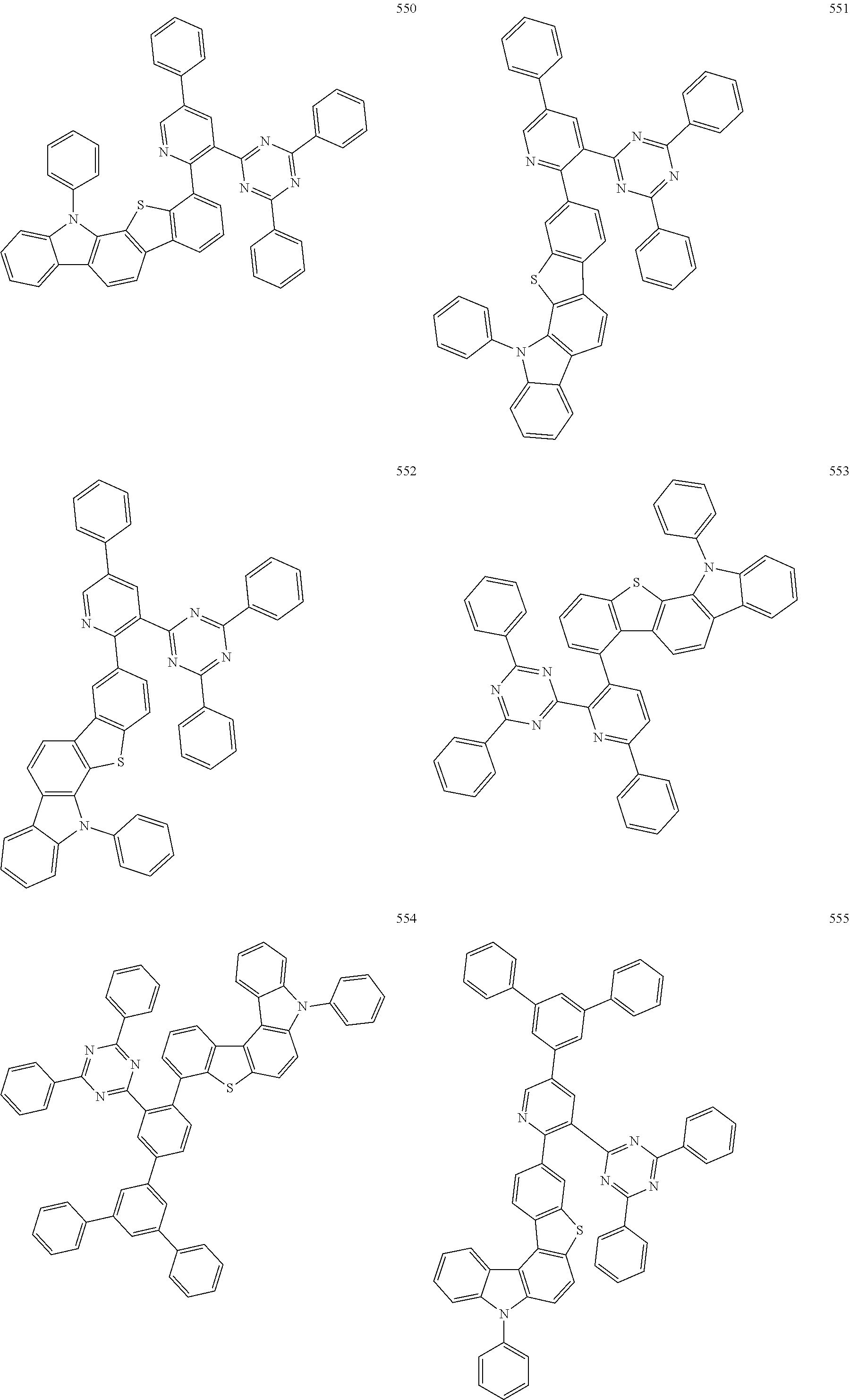

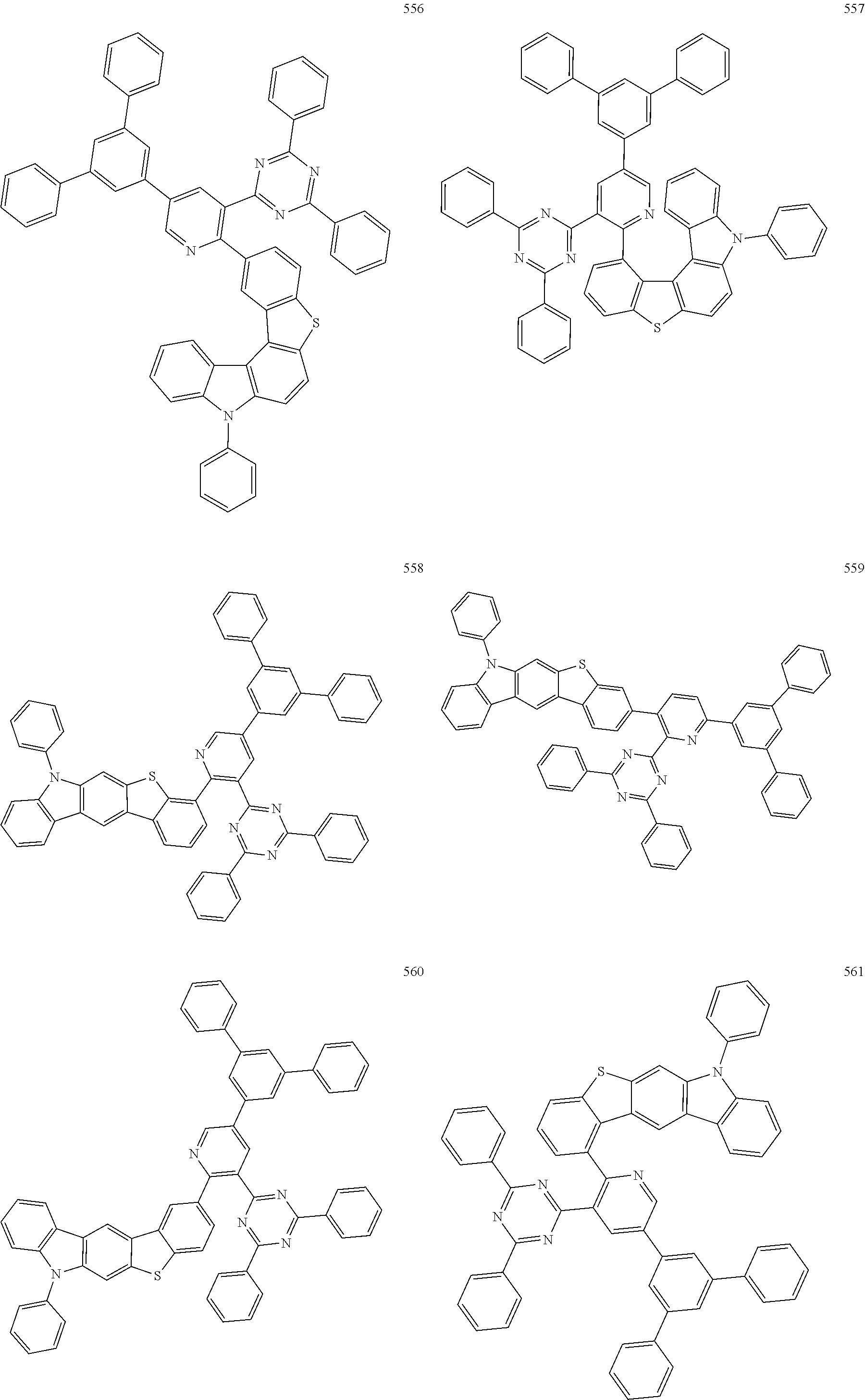

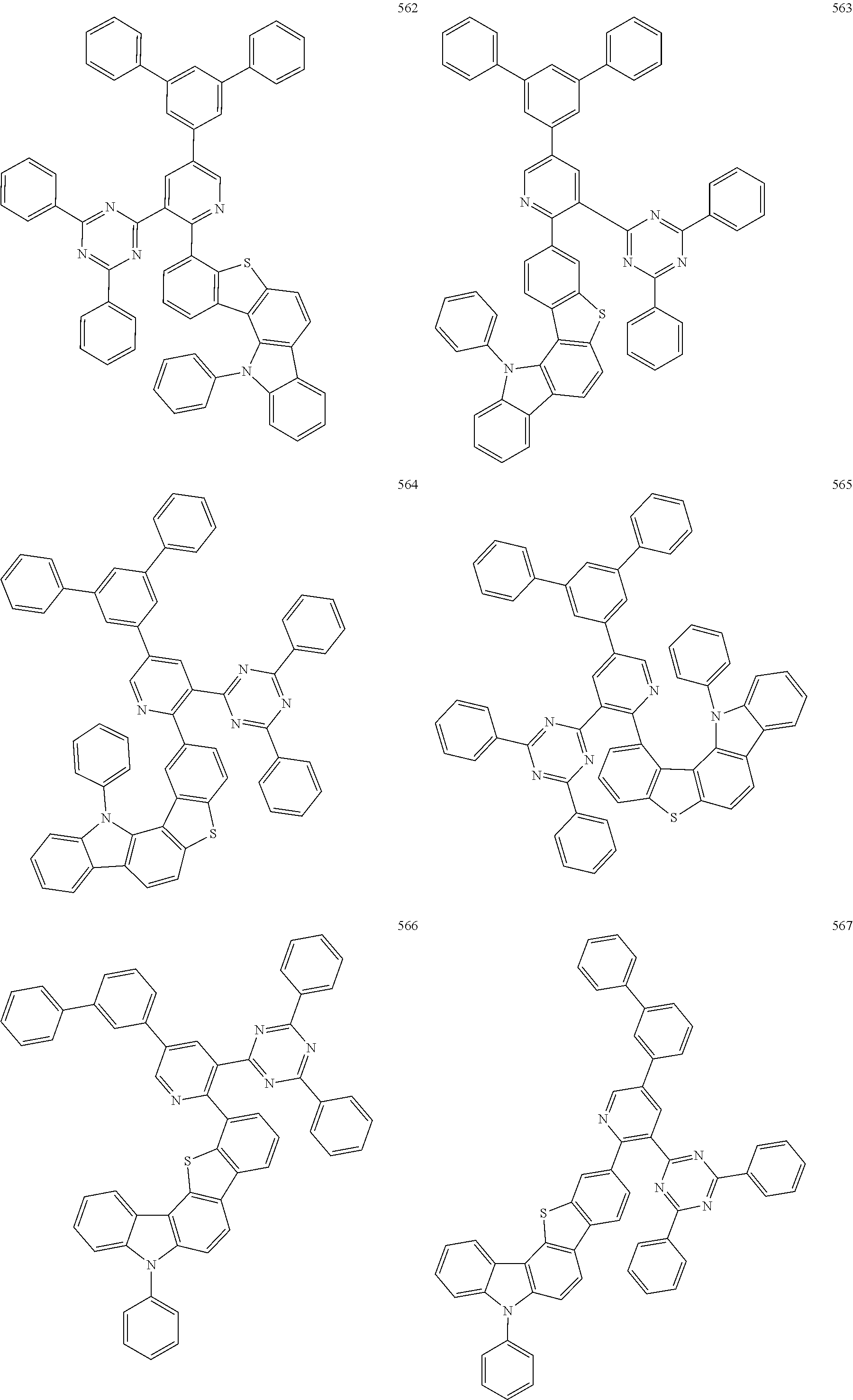

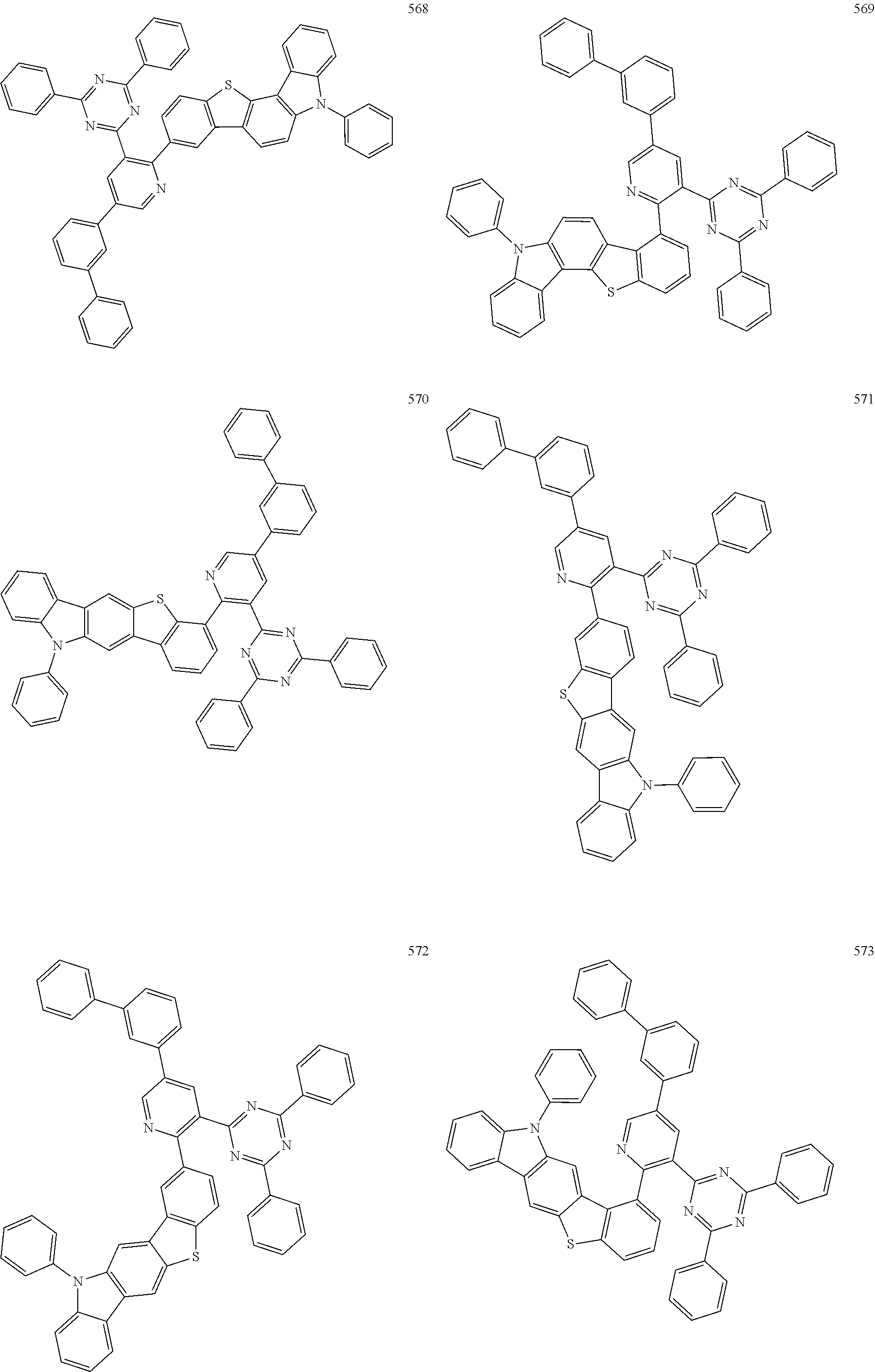

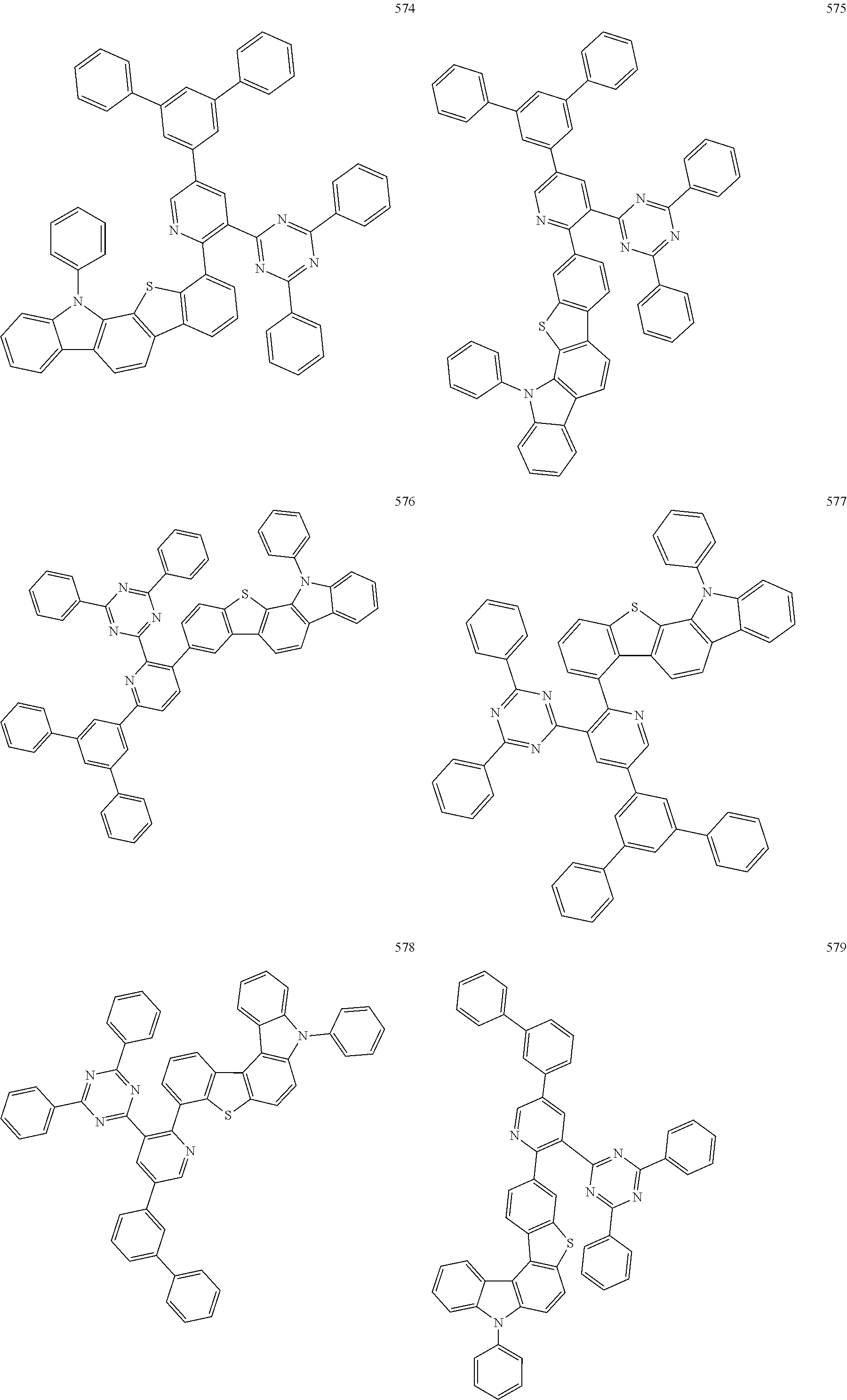

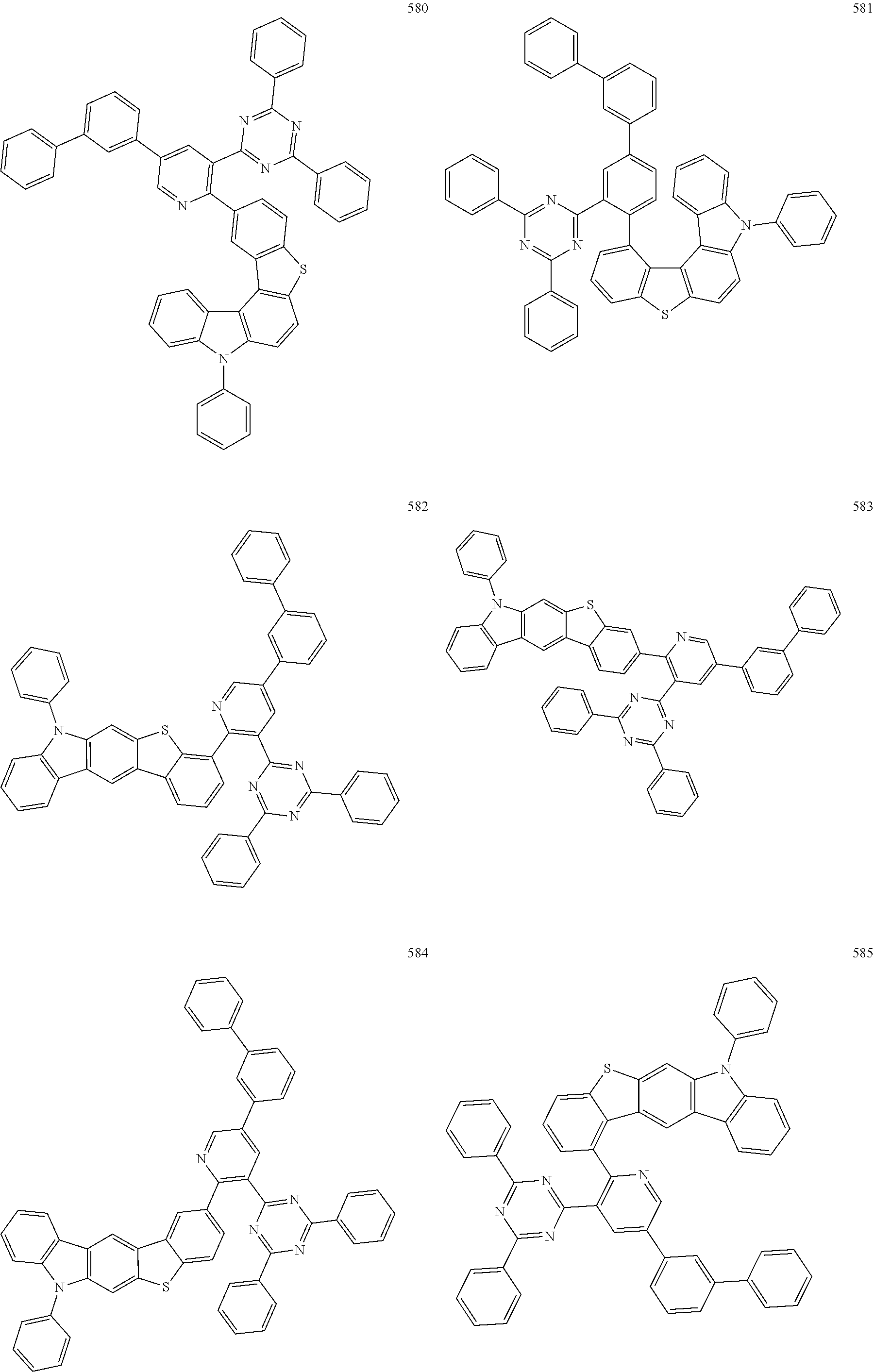

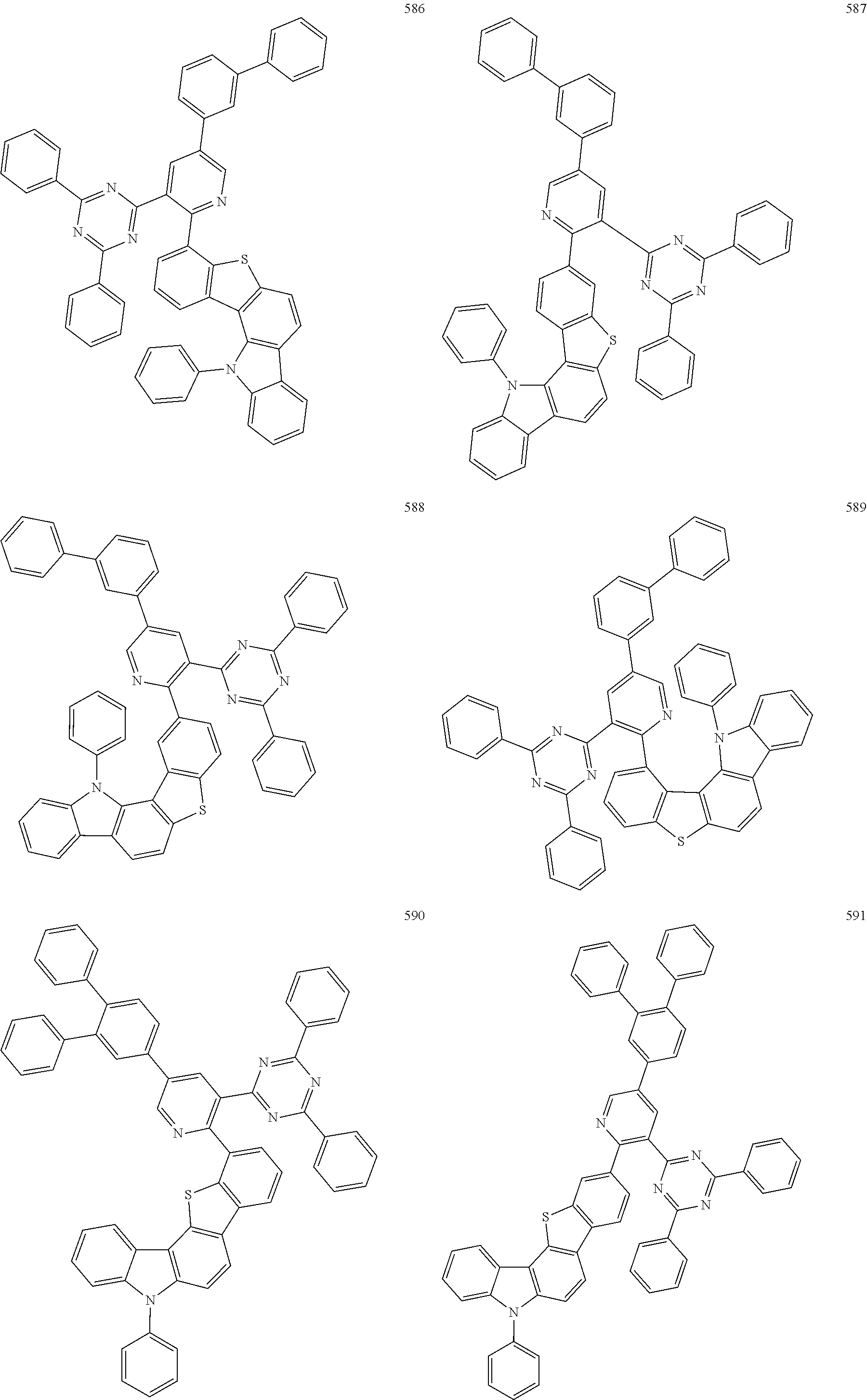

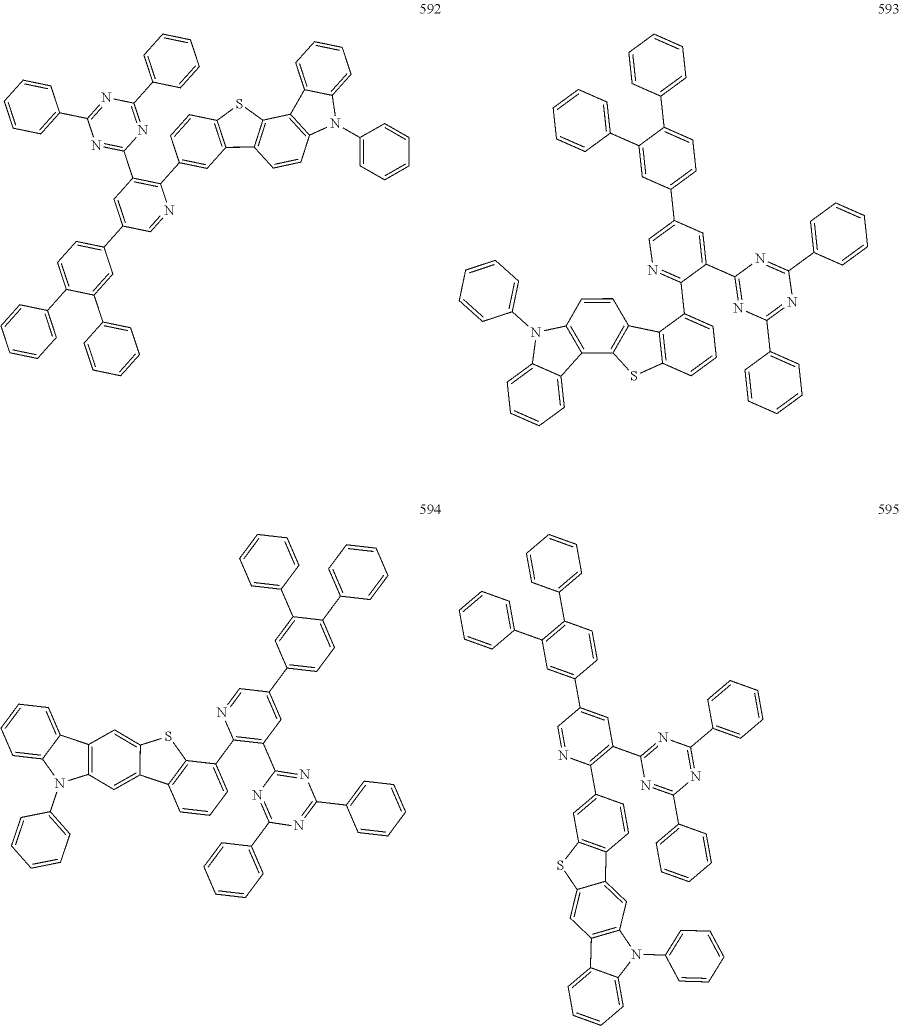 [SMILES: c1ccc(-c2nc(-c3ccccc3)nc(-c3cc(-c4ccc(-c5ccccc5)c(-c5ccccc5)c4)cnc3-c3ccc4sc5c(ccc6c5c5ccccc5n6-c5ccccc5)c4c3)n2)cc1 ; c1ccc(-c2nc(-c3ccccc3)nc(-c3cc(-c4ccc(-c5ccccc5)c(-c5ccccc5)c4)cnc3-c3cccc4sc5c(ccc6c5c5ccccc5n6-c5ccccc5)c34)n2)cc1 ; c1ccc(-c2nc(-c3ccccc3)nc(-c3cc(-c4ccc(-c5ccccc5)c(-c5ccccc5)c4)cnc3-c3cccc4c3sc3cc5c6ccccc6n(-c6ccccc6)c5cc34)n2)cc1 ; c1ccc(-c2nc(-c3ccccc3)nc(-c3cc(-c4ccc(-c5ccccc5)c(-c5ccccc5)c4)cnc3-c3ccc4c(c3)sc3cc5c6ccccc6n(-c6ccccc6)c5cc34)n2)cc1]

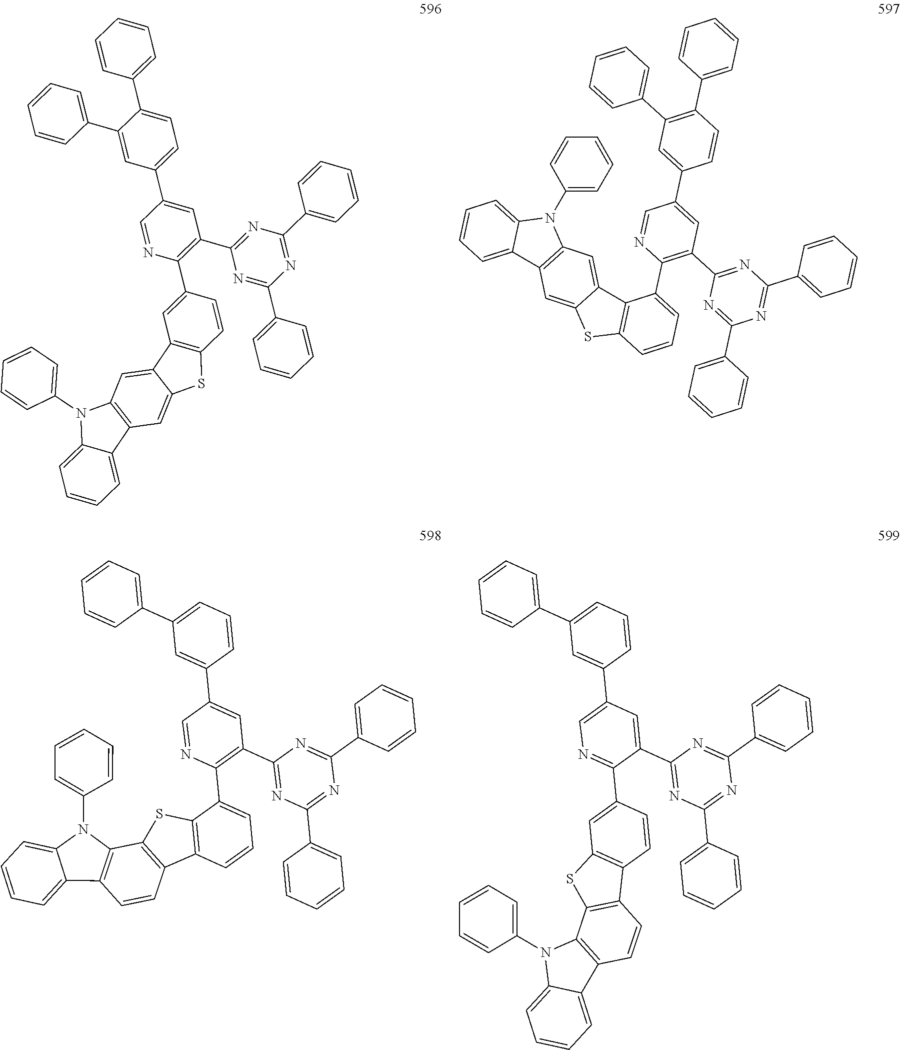 [SMILES: c1ccc(-c2nc(-c3ccccc3)nc(-c3cc(-c4ccc(-c5ccccc5)c(-c5ccccc5)c4)cnc3-c3ccc4sc5cc6c7ccccc7n(-c7ccccc7)c6cc5c4c3)n2)cc1 ; c1ccc(-c2nc(-c3ccccc3)nc(-c3cc(-c4ccc(-c5ccccc5)c(-c5ccccc5)c4)cnc3-c3cccc4sc5cc6c7ccccc7n(-c7ccccc7)c6cc5c34)n2)cc1 ; c1ccc(-c2cccc(-c3cnc(-c4cccc5c4sc4c5ccc5c6ccccc6n(-c6ccccc6)c54)c(-c4nc(-c5ccccc5)nc(-c5ccccc5)n4)c3)c2)cc1 ; c1ccc(-c2cccc(-c3cnc(-c4ccc5c(c4)sc4c5ccc5c6ccccc6n(-c6ccccc6)c54)c(-c4nc(-c5ccccc5)nc(-c5ccccc5)n4)c3)c2)cc1]

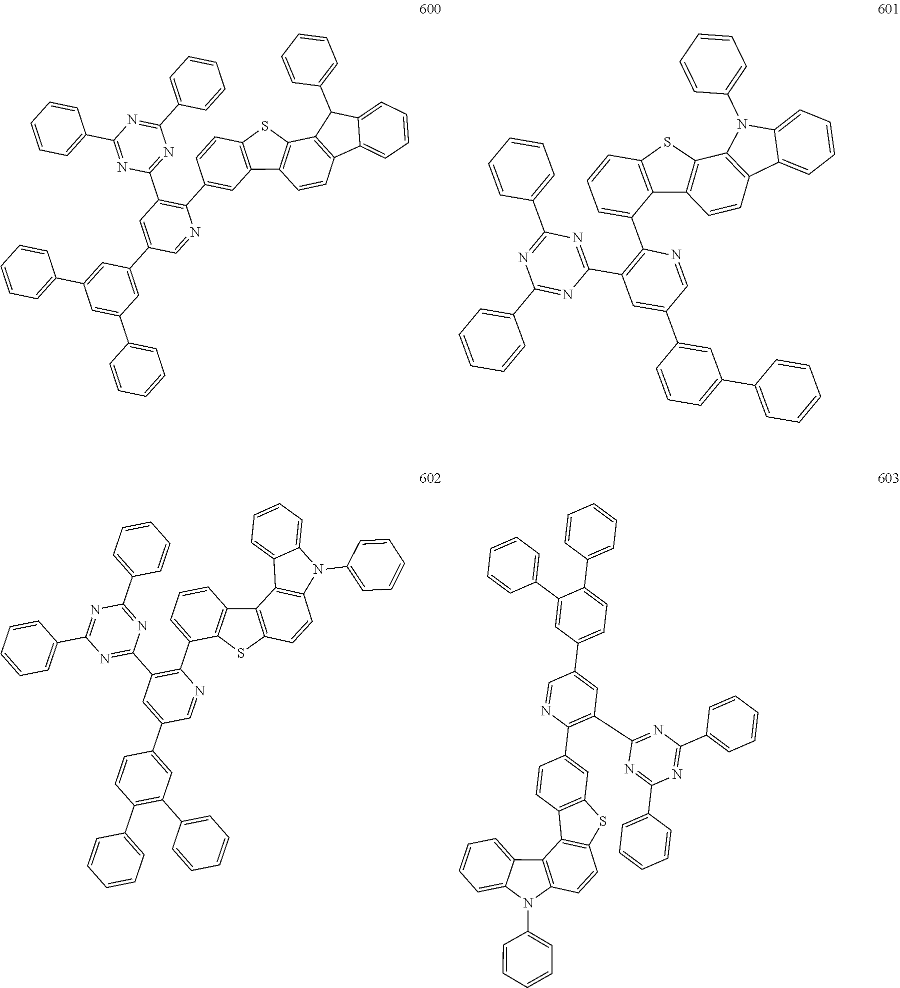 [SMILES: c1ccc(-c2cc(-c3ccccc3)cc(-c3cnc(-c4ccc5sc6c7c(ccc6c5c4)-c4ccccc4C7c4ccccc4)c(-c4nc(-c5ccccc5)nc(-c5ccccc5)n4)c3)c2)cc1 ; c1ccc(-c2cccc(-c3cnc(-c4cccc5sc6c(ccc7c8ccccc8n(-c8ccccc8)c76)c45)c(-c4nc(-c5ccccc5)nc(-c5ccccc5)n4)c3)c2)cc1 ; c1ccc(-c2nc(-c3ccccc3)nc(-c3cc(-c4ccc(-c5ccccc5)c(-c5ccccc5)c4)cnc3-c3cccc4c3sc3ccc5c(c6ccccc6n5-c5ccccc5)c34)n2)cc1 ; c1ccc(-c2nc(-c3ccccc3)nc(-c3cc(-c4ccc(-c5ccccc5)c(-c5ccccc5)c4)cnc3-c3ccc4c(c3)sc3ccc5c(c6ccccc6n5-c5ccccc5)c34)n2)cc1]

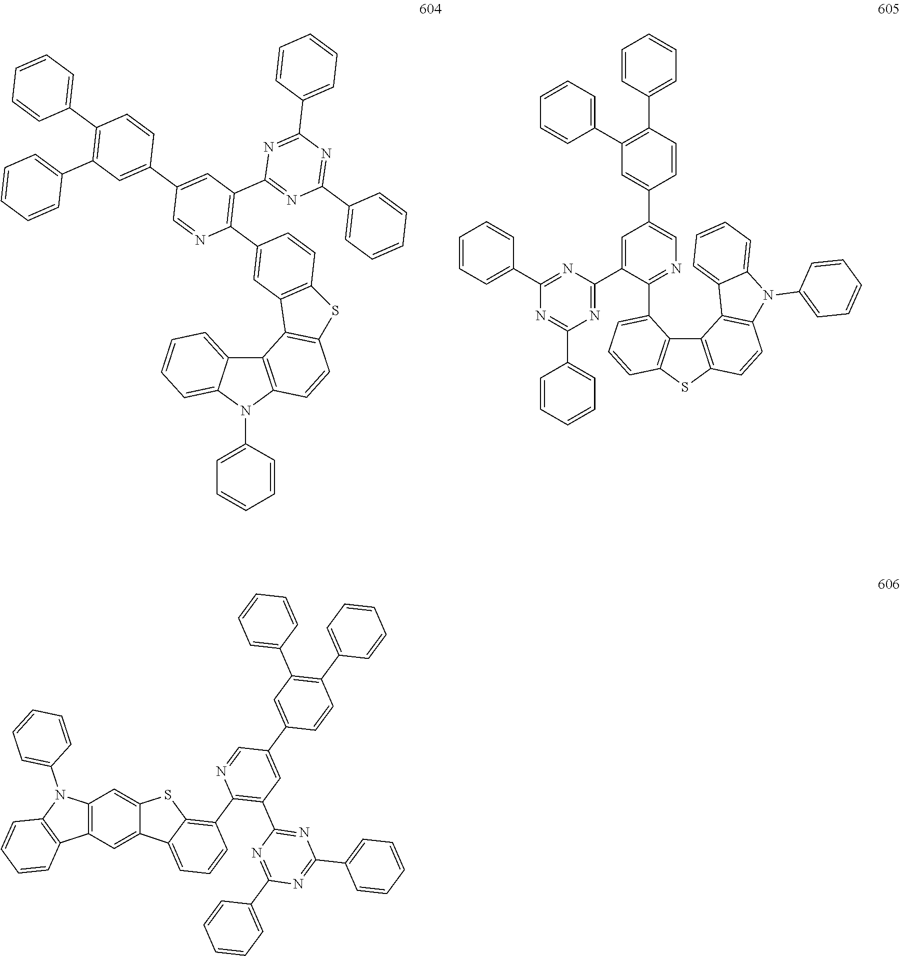 [SMILES: c1ccc(-c2nc(-c3ccccc3)nc(-c3cc(-c4ccc(-c5ccccc5)c(-c5ccccc5)c4)cnc3-c3ccc4sc5ccc6c(c7ccccc7n6-c6ccccc6)c5c4c3)n2)cc1 ; c1ccc(-c2nc(-c3ccccc3)nc(-c3cc(-c4ccc(-c5ccccc5)c(-c5ccccc5)c4)cnc3-c3cccc4sc5ccc6c(c7ccccc7n6-c6ccccc6)c5c34)n2)cc1 ; c1ccc(-c2nc(-c3ccccc3)nc(-c3cc(-c4ccc(-c5ccccc5)c(-c5ccccc5)c4)cnc3-c3cccc4c3sc3cc5c(cc34)c3ccccc3n5-c3ccccc3)n2)cc1]

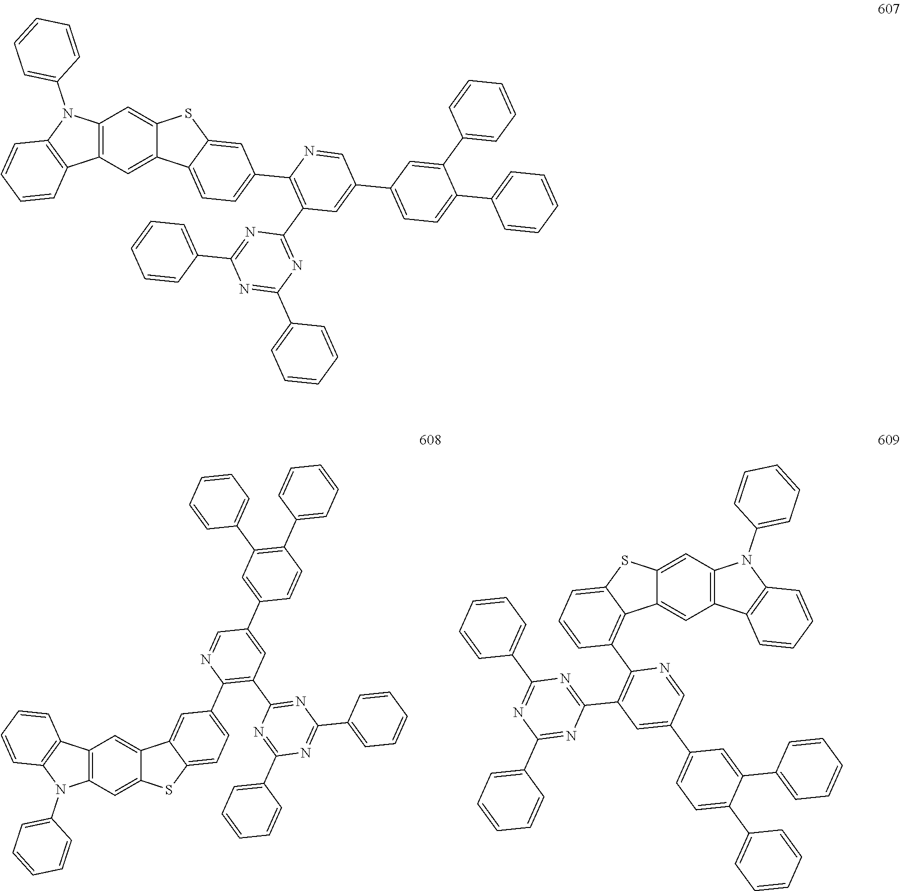 [SMILES: c1ccc(-c2nc(-c3ccccc3)nc(-c3cc(-c4ccc(-c5ccccc5)c(-c5ccccc5)c4)cnc3-c3ccc4c(c3)sc3cc5c(cc34)c3ccccc3n5-c3ccccc3)n2)cc1 ; c1ccc(-c2nc(-c3ccccc3)nc(-c3cc(-c4ccc(-c5ccccc5)c(-c5ccccc5)c4)cnc3-c3ccc4sc5cc6c(cc5c4c3)c3ccccc3n6-c3ccccc3)n2)cc1 ; c1ccc(-c2nc(-c3ccccc3)nc(-c3cc(-c4ccc(-c5ccccc5)c(-c5ccccc5)c4)cnc3-c3cccc4sc5cc6c(cc5c34)c3ccccc3n6-c3ccccc3)n2)cc1]

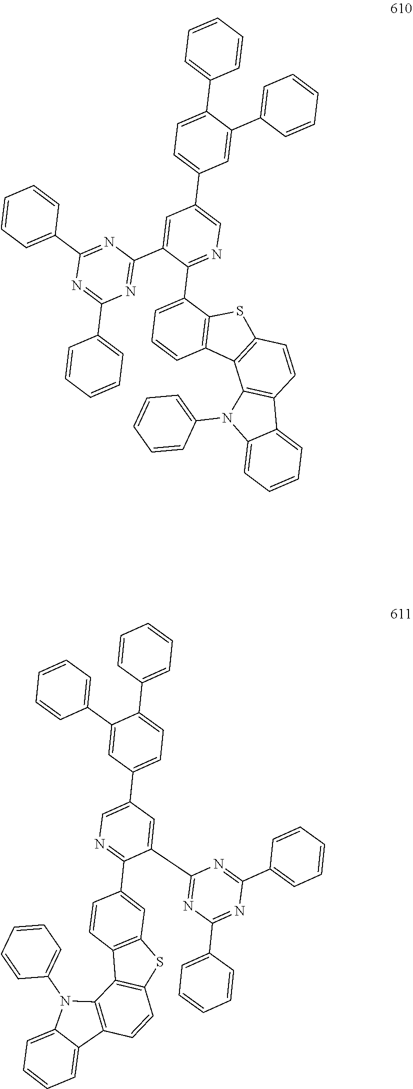 [SMILES: c1ccc(-c2nc(-c3ccccc3)nc(-c3cc(-c4ccc(-c5ccccc5)c(-c5ccccc5)c4)cnc3-c3cccc4c3sc3ccc5c6ccccc6n(-c6ccccc6)c5c34)n2)cc1 ; c1ccc(-c2nc(-c3ccccc3)nc(-c3cc(-c4ccc(-c5ccccc5)c(-c5ccccc5)c4)cnc3-c3ccc4c(c3)sc3ccc5c6ccccc6n(-c6ccccc6)c5c34)n2)cc1]

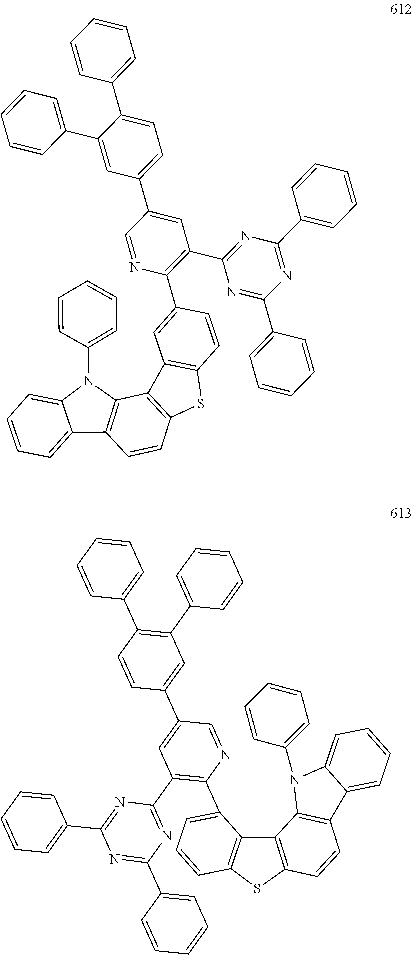 [SMILES: c1ccc(-c2nc(-c3ccccc3)nc(-c3cc(-c4ccc(-c5ccccc5)c(-c5ccccc5)c4)cnc3-c3ccc4sc5ccc6c7ccccc7n(-c7ccccc7)c6c5c4c3)n2)cc1 ; c1ccc(-c2nc(-c3ccccc3)nc(-c3cc(-c4ccc(-c5ccccc5)c(-c5ccccc5)c4)cnc3-c3cccc4sc5ccc6c7ccccc7n(-c7ccccc7)c6c5c34)n2)cc1]

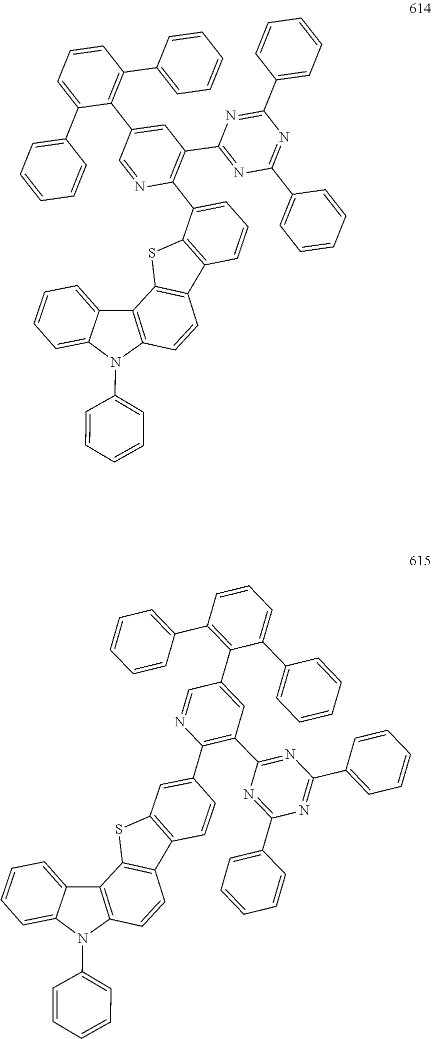 [SMILES: c1ccc(-c2nc(-c3ccccc3)nc(-c3cc(-c4c(-c5ccccc5)cccc4-c4ccccc4)cnc3-c3cccc4c3sc3c4ccc4c3c3ccccc3n4-c3ccccc3)n2)cc1 ; c1ccc(-c2nc(-c3ccccc3)nc(-c3cc(-c4c(-c5ccccc5)cccc4-c4ccccc4)cnc3-c3ccc4c(c3)sc3c4ccc4c3c3ccccc3n4-c3ccccc3)n2)cc1]

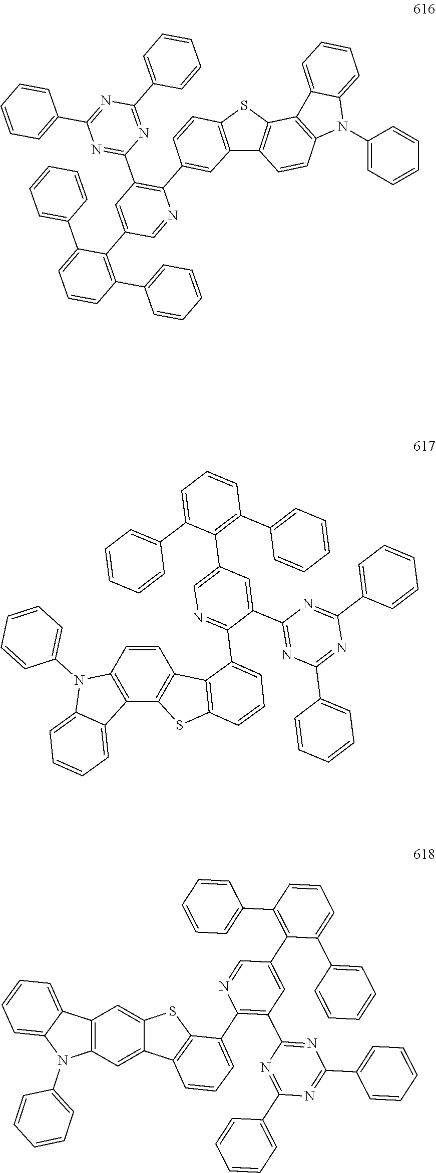 [SMILES: c1ccc(-c2nc(-c3ccccc3)nc(-c3cc(-c4c(-c5ccccc5)cccc4-c4ccccc4)cnc3-c3ccc4sc5c(ccc6c5c5ccccc5n6-c5ccccc5)c4c3)n2)cc1 ; c1ccc(-c2nc(-c3ccccc3)nc(-c3cc(-c4c(-c5ccccc5)cccc4-c4ccccc4)cnc3-c3cccc4sc5c(ccc6c5c5ccccc5n6-c5ccccc5)c34)n2)cc1 ; c1ccc(-c2nc(-c3ccccc3)nc(-c3cc(-c4c(-c5ccccc5)cccc4-c4ccccc4)cnc3-c3cccc4c3sc3cc5c6ccccc6n(-c6ccccc6)c5cc34)n2)cc1]

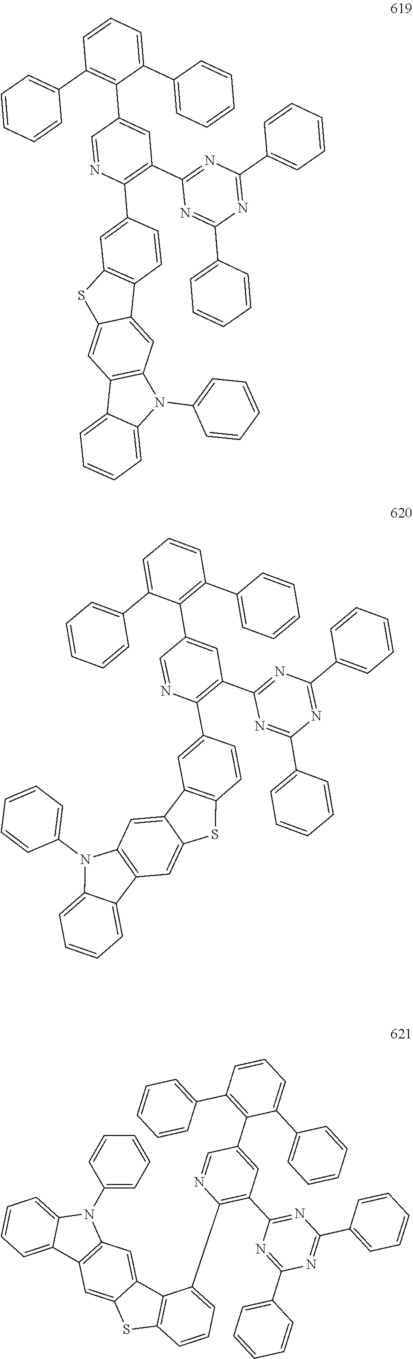 [SMILES: c1ccc(-c2nc(-c3ccccc3)nc(-c3cc(-c4c(-c5ccccc5)cccc4-c4ccccc4)cnc3-c3ccc4c(c3)sc3cc5c6ccccc6n(-c6ccccc6)c5cc34)n2)cc1 ; c1ccc(-c2nc(-c3ccccc3)nc(-c3cc(-c4c(-c5ccccc5)cccc4-c4ccccc4)cnc3-c3ccc4sc5cc6c7ccccc7n(-c7ccccc7)c6cc5c4c3)n2)cc1 ; c1ccc(-c2nc(-c3ccccc3)nc(-c3cc(-c4c(-c5ccccc5)cccc4-c4ccccc4)cnc3-c3cccc4sc5cc6c7ccccc7n(-c7ccccc7)c6cc5c34)n2)cc1]

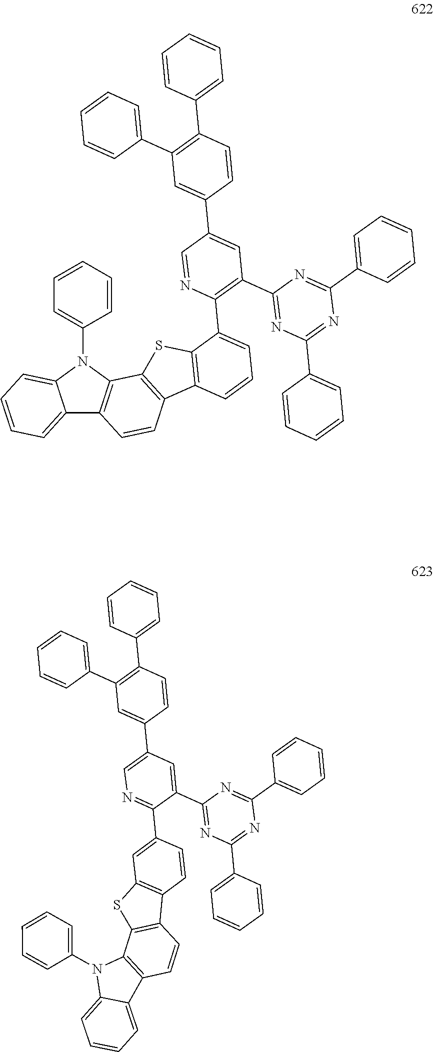 [SMILES: c1ccc(-c2nc(-c3ccccc3)nc(-c3cc(-c4ccc(-c5ccccc5)c(-c5ccccc5)c4)cnc3-c3cccc4c3sc3c4ccc4c5ccccc5n(-c5ccccc5)c43)n2)cc1 ; c1ccc(-c2nc(-c3ccccc3)nc(-c3cc(-c4ccc(-c5ccccc5)c(-c5ccccc5)c4)cnc3-c3ccc4c(c3)sc3c4ccc4c5ccccc5n(-c5ccccc5)c43)n2)cc1]

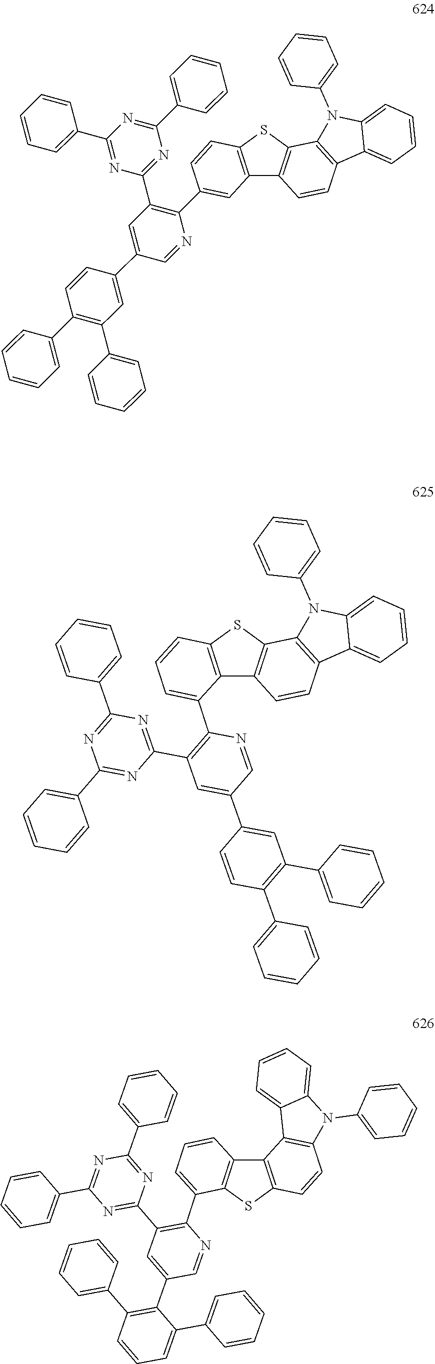 [SMILES: c1ccc(-c2nc(-c3ccccc3)nc(-c3cc(-c4ccc(-c5ccccc5)c(-c5ccccc5)c4)cnc3-c3ccc4sc5c(ccc6c7ccccc7n(-c7ccccc7)c65)c4c3)n2)cc1 ; c1ccc(-c2nc(-c3ccccc3)nc(-c3cc(-c4ccc(-c5ccccc5)c(-c5ccccc5)c4)cnc3-c3cccc4sc5c(ccc6c7ccccc7n(-c7ccccc7)c65)c34)n2)cc1 ; c1ccc(-c2nc(-c3ccccc3)nc(-c3cc(-c4c(-c5ccccc5)cccc4-c4ccccc4)cnc3-c3cccc4c3sc3ccc5c(c6ccccc6n5-c5ccccc5)c34)n2)cc1]

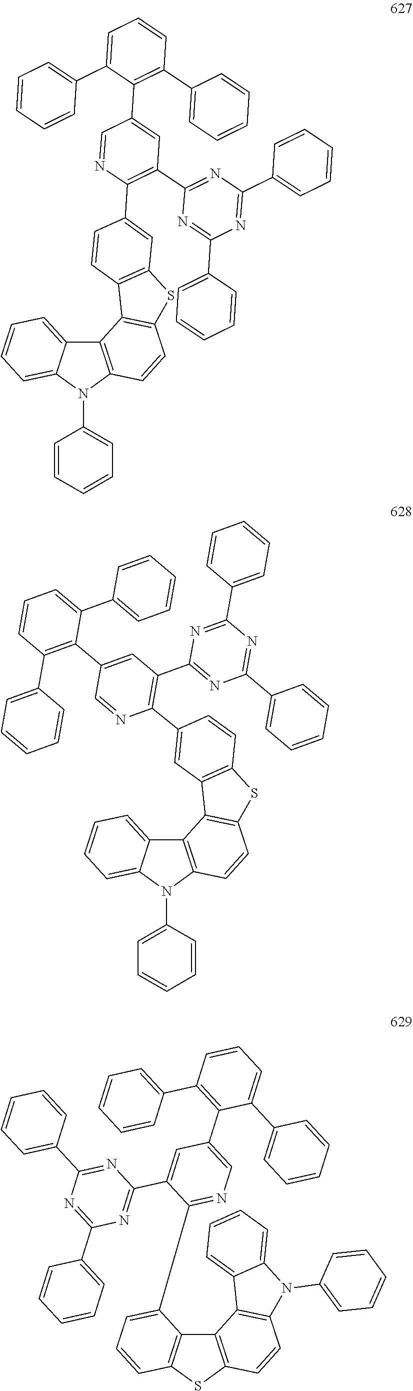 [SMILES: c1ccc(-c2nc(-c3ccccc3)nc(-c3cc(-c4c(-c5ccccc5)cccc4-c4ccccc4)cnc3-c3ccc4c(c3)sc3ccc5c(c6ccccc6n5-c5ccccc5)c34)n2)cc1 ; c1ccc(-c2nc(-c3ccccc3)nc(-c3cc(-c4c(-c5ccccc5)cccc4-c4ccccc4)cnc3-c3ccc4sc5ccc6c(c7ccccc7n6-c6ccccc6)c5c4c3)n2)cc1 ; c1ccc(-c2nc(-c3ccccc3)nc(-c3cc(-c4c(-c5ccccc5)cccc4-c4ccccc4)cnc3-c3cccc4sc5ccc6c(c7ccccc7n6-c6ccccc6)c5c34)n2)cc1]

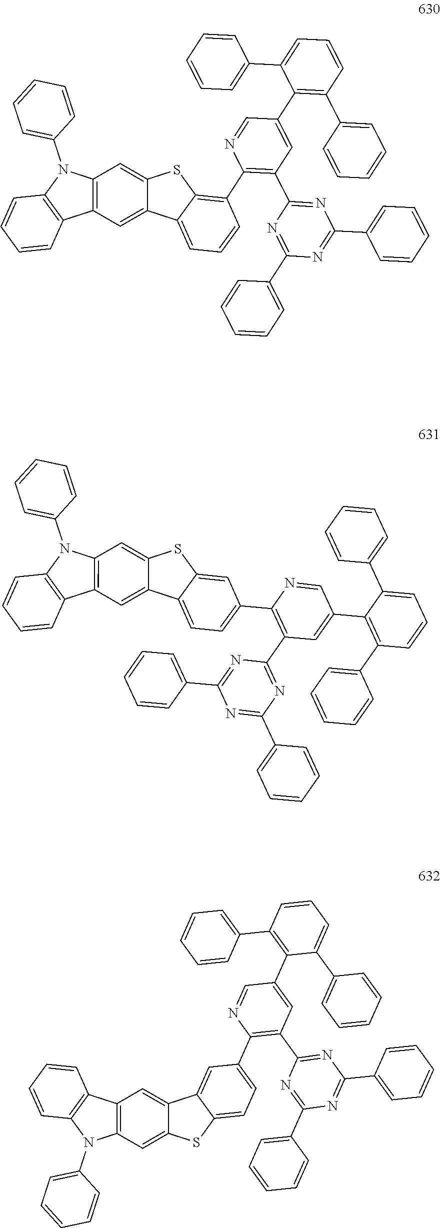 [SMILES: c1ccc(-c2nc(-c3ccccc3)nc(-c3cc(-c4c(-c5ccccc5)cccc4-c4ccccc4)cnc3-c3cccc4c3sc3cc5c(cc34)c3ccccc3n5-c3ccccc3)n2)cc1 ; c1ccc(-c2nc(-c3ccccc3)nc(-c3cc(-c4c(-c5ccccc5)cccc4-c4ccccc4)cnc3-c3ccc4c(c3)sc3cc5c(cc34)c3ccccc3n5-c3ccccc3)n2)cc1 ; c1ccc(-c2nc(-c3ccccc3)nc(-c3cc(-c4c(-c5ccccc5)cccc4-c4ccccc4)cnc3-c3ccc4sc5cc6c(cc5c4c3)c3ccccc3n6-c3ccccc3)n2)cc1]

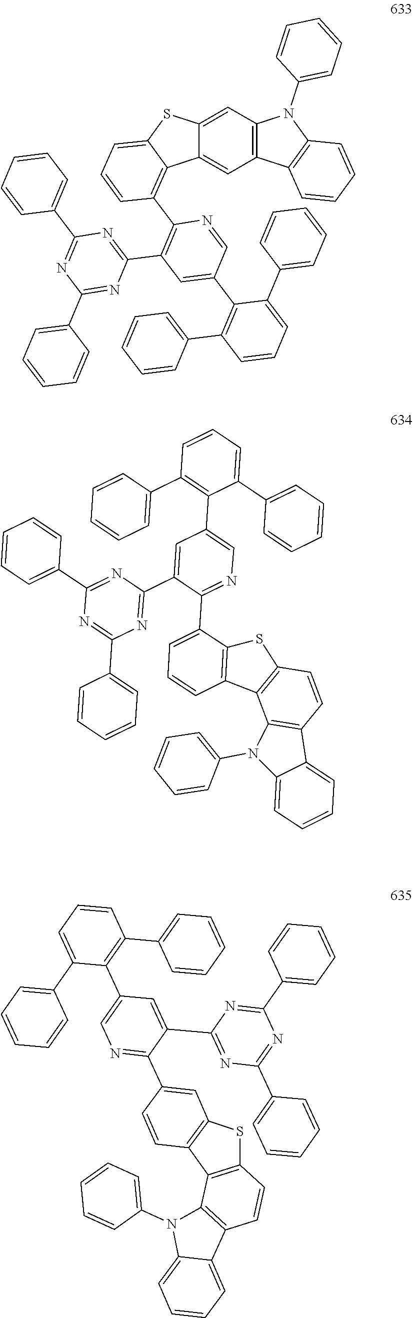 [SMILES: c1ccc(-c2nc(-c3ccccc3)nc(-c3cc(-c4c(-c5ccccc5)cccc4-c4ccccc4)cnc3-c3cccc4sc5cc6c(cc5c34)c3ccccc3n6-c3ccccc3)n2)cc1 ; c1ccc(-c2nc(-c3ccccc3)nc(-c3cc(-c4c(-c5ccccc5)cccc4-c4ccccc4)cnc3-c3cccc4c3sc3ccc5c6ccccc6n(-c6ccccc6)c5c34)n2)cc1 ; c1ccc(-c2nc(-c3ccccc3)nc(-c3cc(-c4c(-c5ccccc5)cccc4-c4ccccc4)cnc3-c3ccc4c(c3)sc3ccc5c6ccccc6n(-c6ccccc6)c5c34)n2)cc1]

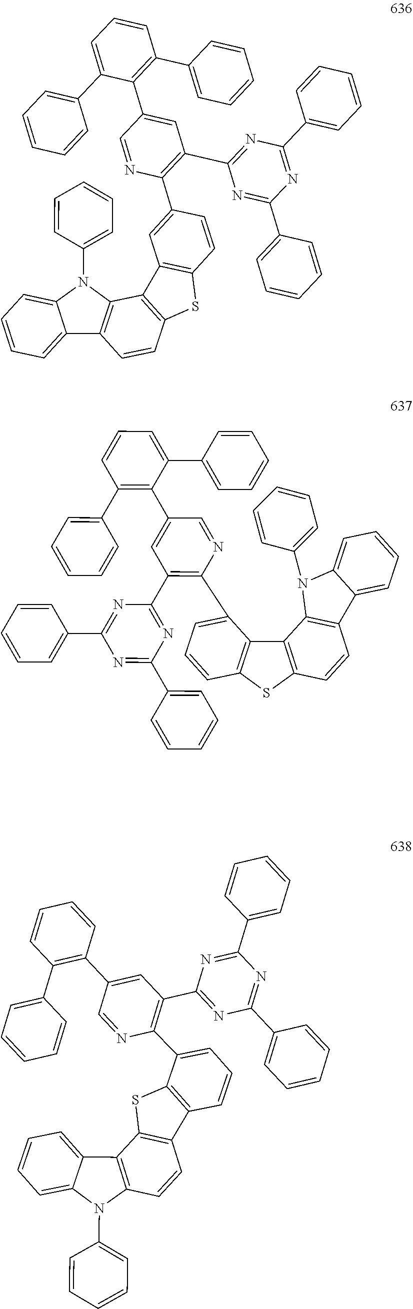 [SMILES: c1ccc(-c2nc(-c3ccccc3)nc(-c3cc(-c4c(-c5ccccc5)cccc4-c4ccccc4)cnc3-c3ccc4sc5ccc6c7ccccc7n(-c7ccccc7)c6c5c4c3)n2)cc1 ; c1ccc(-c2nc(-c3ccccc3)nc(-c3cc(-c4c(-c5ccccc5)cccc4-c4ccccc4)cnc3-c3cccc4sc5ccc6c7ccccc7n(-c7ccccc7)c6c5c34)n2)cc1 ; c1ccc(-c2nc(-c3ccccc3)nc(-c3cc(-c4ccccc4-c4ccccc4)cnc3-c3cccc4c3sc3c4ccc4c3c3ccccc3n4-c3ccccc3)n2)cc1]

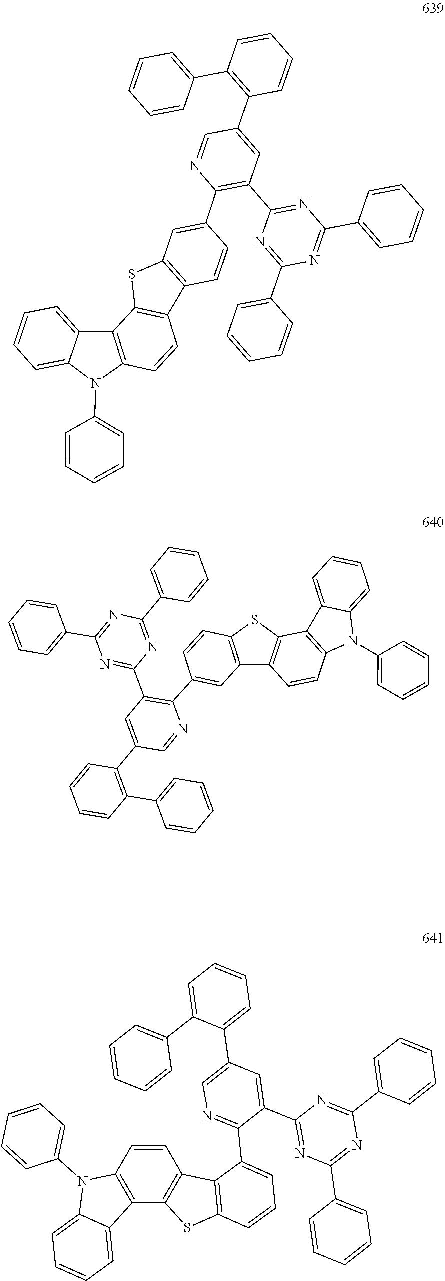 [SMILES: c1ccc(-c2nc(-c3ccccc3)nc(-c3cc(-c4ccccc4-c4ccccc4)cnc3-c3ccc4c(c3)sc3c4ccc4c3c3ccccc3n4-c3ccccc3)n2)cc1 ; c1ccc(-c2nc(-c3ccccc3)nc(-c3cc(-c4ccccc4-c4ccccc4)cnc3-c3ccc4sc5c(ccc6c5c5ccccc5n6-c5ccccc5)c4c3)n2)cc1 ; c1ccc(-c2nc(-c3ccccc3)nc(-c3cc(-c4ccccc4-c4ccccc4)cnc3-c3cccc4sc5c(ccc6c5c5ccccc5n6-c5ccccc5)c34)n2)cc1]

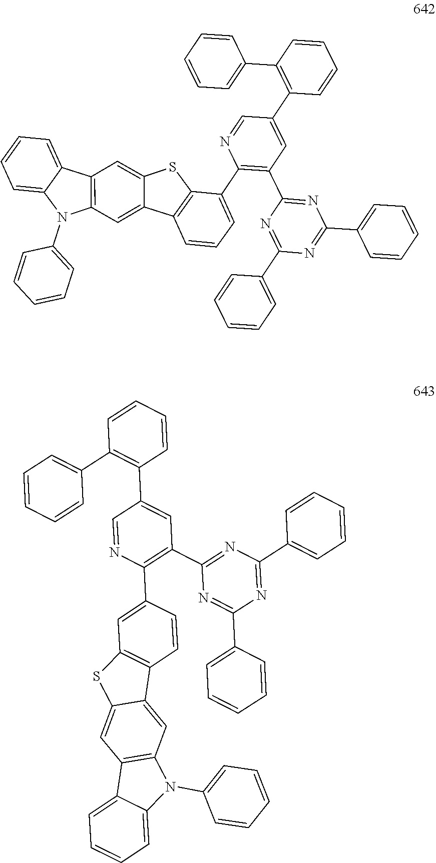 [SMILES: c1ccc(-c2nc(-c3ccccc3)nc(-c3cc(-c4ccccc4-c4ccccc4)cnc3-c3cccc4c3sc3cc5c6ccccc6n(-c6ccccc6)c5cc34)n2)cc1 ; c1ccc(-c2nc(-c3ccccc3)nc(-c3cc(-c4ccccc4-c4ccccc4)cnc3-c3ccc4c(c3)sc3cc5c6ccccc6n(-c6ccccc6)c5cc34)n2)cc1]

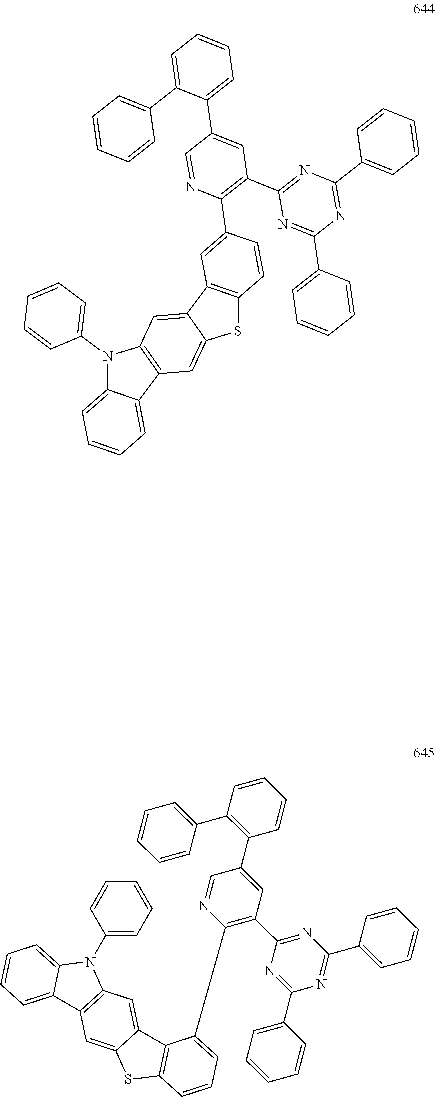 [SMILES: c1ccc(-c2nc(-c3ccccc3)nc(-c3cc(-c4ccccc4-c4ccccc4)cnc3-c3ccc4sc5cc6c7ccccc7n(-c7ccccc7)c6cc5c4c3)n2)cc1 ; c1ccc(-c2nc(-c3ccccc3)nc(-c3cc(-c4ccccc4-c4ccccc4)cnc3-c3cccc4sc5cc6c7ccccc7n(-c7ccccc7)c6cc5c34)n2)cc1]

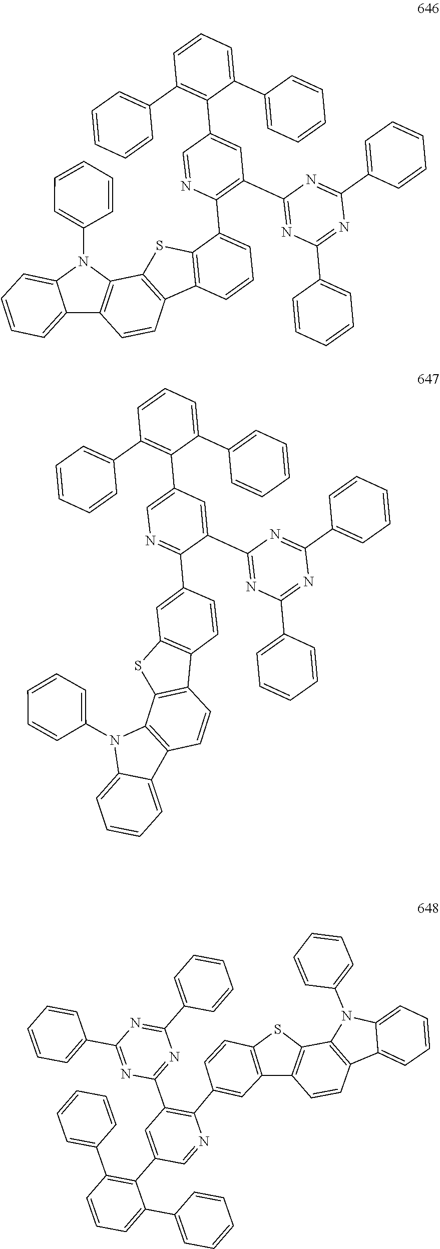 [SMILES: c1ccc(-c2nc(-c3ccccc3)nc(-c3cc(-c4c(-c5ccccc5)cccc4-c4ccccc4)cnc3-c3cccc4c3sc3c4ccc4c5ccccc5n(-c5ccccc5)c43)n2)cc1 ; c1ccc(-c2nc(-c3ccccc3)nc(-c3cc(-c4c(-c5ccccc5)cccc4-c4ccccc4)cnc3-c3ccc4c(c3)sc3c4ccc4c5ccccc5n(-c5ccccc5)c43)n2)cc1 ; c1ccc(-c2nc(-c3ccccc3)nc(-c3cc(-c4c(-c5ccccc5)cccc4-c4ccccc4)cnc3-c3ccc4sc5c(ccc6c7ccccc7n(-c7ccccc7)c65)c4c3)n2)cc1]

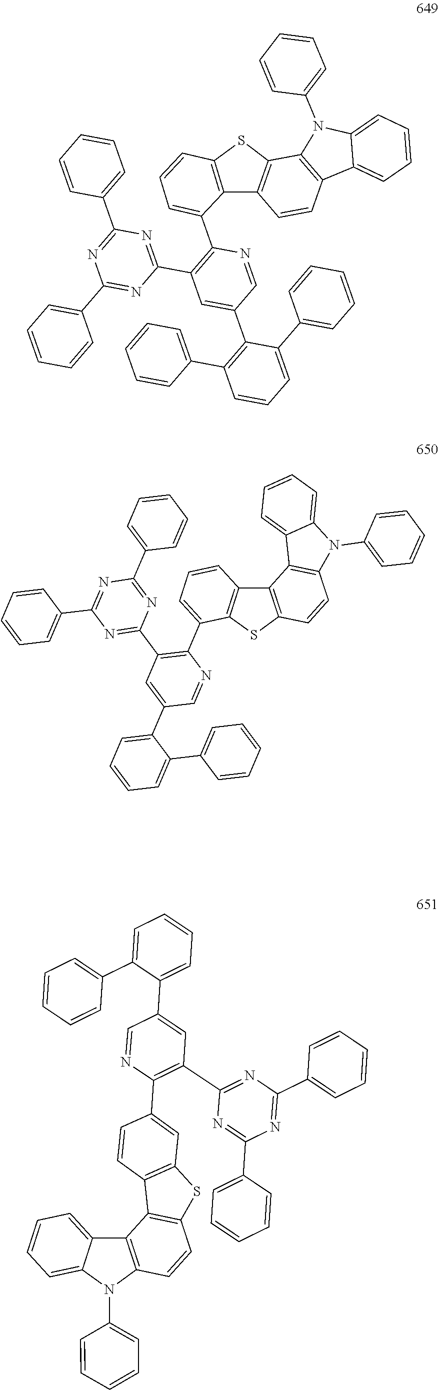 [SMILES: c1ccc(-c2nc(-c3ccccc3)nc(-c3cc(-c4c(-c5ccccc5)cccc4-c4ccccc4)cnc3-c3cccc4sc5c(ccc6c7ccccc7n(-c7ccccc7)c65)c34)n2)cc1 ; c1ccc(-c2nc(-c3ccccc3)nc(-c3cc(-c4ccccc4-c4ccccc4)cnc3-c3cccc4c3sc3ccc5c(c6ccccc6n5-c5ccccc5)c34)n2)cc1 ; c1ccc(-c2nc(-c3ccccc3)nc(-c3cc(-c4ccccc4-c4ccccc4)cnc3-c3ccc4c(c3)sc3ccc5c(c6ccccc6n5-c5ccccc5)c34)n2)cc1]

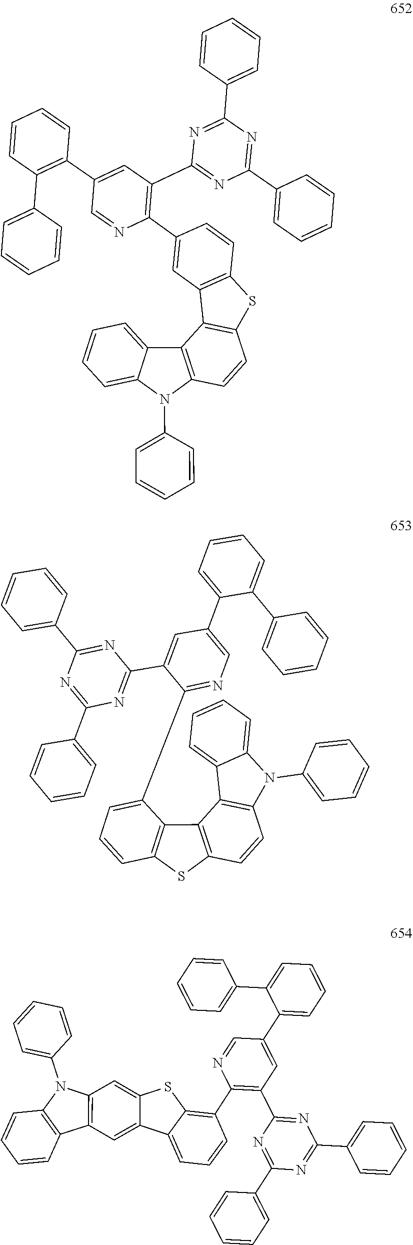 [SMILES: c1ccc(-c2nc(-c3ccccc3)nc(-c3cc(-c4ccccc4-c4ccccc4)cnc3-c3ccc4sc5ccc6c(c7ccccc7n6-c6ccccc6)c5c4c3)n2)cc1 ; c1ccc(-c2nc(-c3ccccc3)nc(-c3cc(-c4ccccc4-c4ccccc4)cnc3-c3cccc4sc5ccc6c(c7ccccc7n6-c6ccccc6)c5c34)n2)cc1 ; c1ccc(-c2nc(-c3ccccc3)nc(-c3cc(-c4ccccc4-c4ccccc4)cnc3-c3cccc4c3sc3cc5c(cc34)c3ccccc3n5-c3ccccc3)n2)cc1]

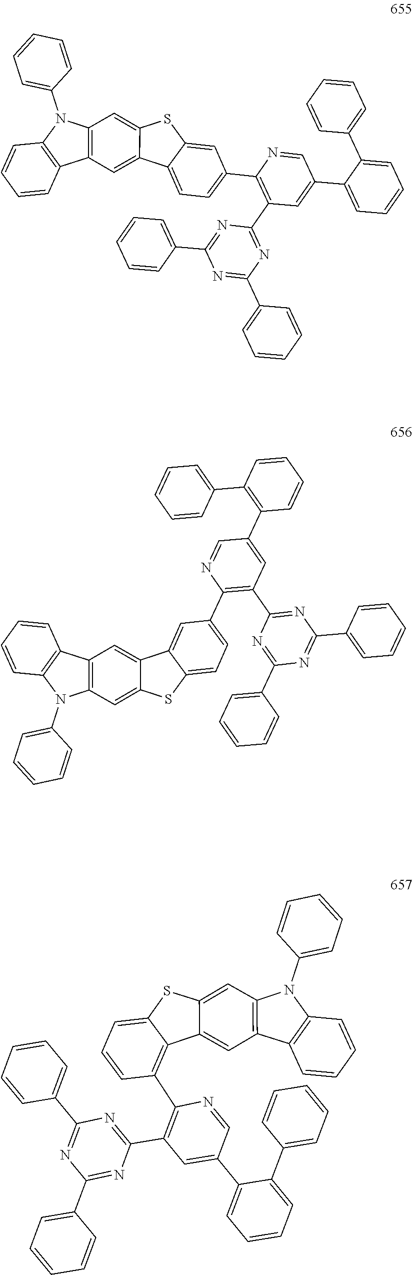 [SMILES: c1ccc(-c2nc(-c3ccccc3)nc(-c3cc(-c4ccccc4-c4ccccc4)cnc3-c3ccc4c(c3)sc3cc5c(cc34)c3ccccc3n5-c3ccccc3)n2)cc1 ; c1ccc(-c2nc(-c3ccccc3)nc(-c3cc(-c4ccccc4-c4ccccc4)cnc3-c3ccc4sc5cc6c(cc5c4c3)c3ccccc3n6-c3ccccc3)n2)cc1 ; c1ccc(-c2nc(-c3ccccc3)nc(-c3cc(-c4ccccc4-c4ccccc4)cnc3-c3cccc4sc5cc6c(cc5c34)c3ccccc3n6-c3ccccc3)n2)cc1]

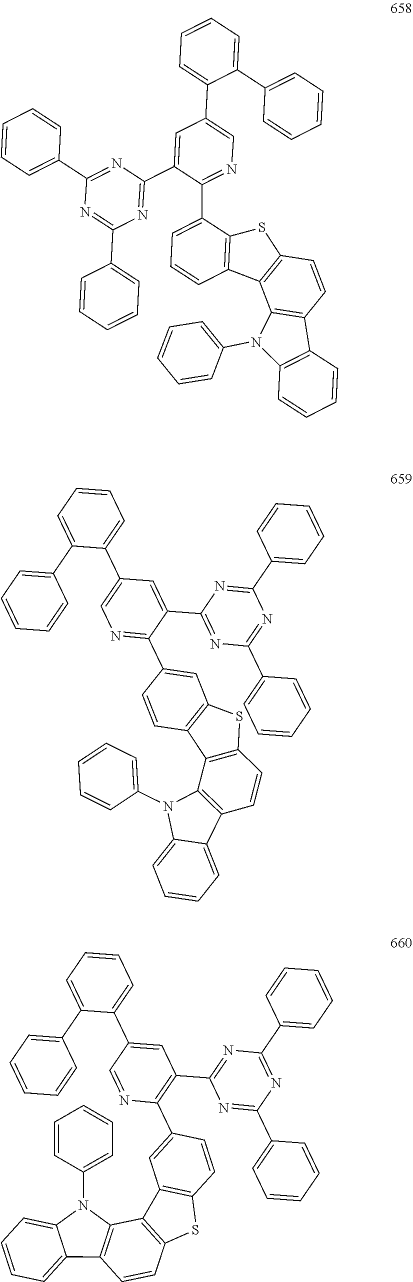 [SMILES: c1ccc(-c2nc(-c3ccccc3)nc(-c3cc(-c4ccccc4-c4ccccc4)cnc3-c3cccc4c3sc3ccc5c6ccccc6n(-c6ccccc6)c5c34)n2)cc1 ; c1ccc(-c2nc(-c3ccccc3)nc(-c3cc(-c4ccccc4-c4ccccc4)cnc3-c3ccc4c(c3)sc3ccc5c6ccccc6n(-c6ccccc6)c5c34)n2)cc1 ; c1ccc(-c2nc(-c3ccccc3)nc(-c3cc(-c4ccccc4-c4ccccc4)cnc3-c3ccc4sc5ccc6c7ccccc7n(-c7ccccc7)c6c5c4c3)n2)cc1]

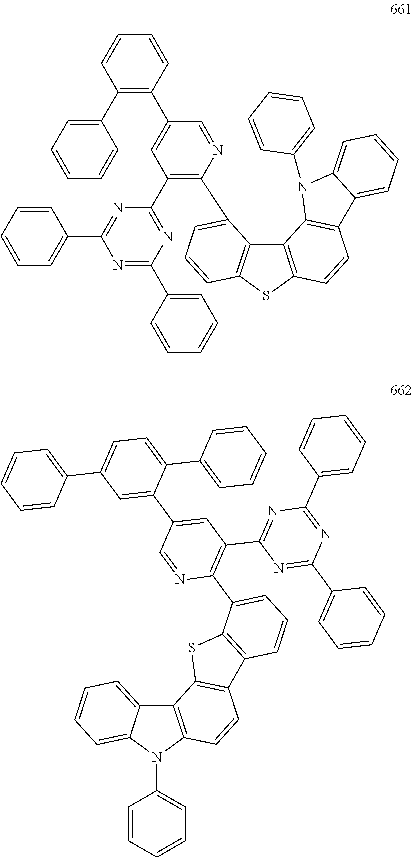 [SMILES: c1ccc(-c2nc(-c3ccccc3)nc(-c3cc(-c4ccccc4-c4ccccc4)cnc3-c3cccc4sc5ccc6c7ccccc7n(-c7ccccc7)c6c5c34)n2)cc1 ; c1ccc(-c2ccc(-c3ccccc3)c(-c3cnc(-c4cccc5c4sc4c5ccc5c4c4ccccc4n5-c4ccccc4)c(-c4nc(-c5ccccc5)nc(-c5ccccc5)n4)c3)c2)cc1]

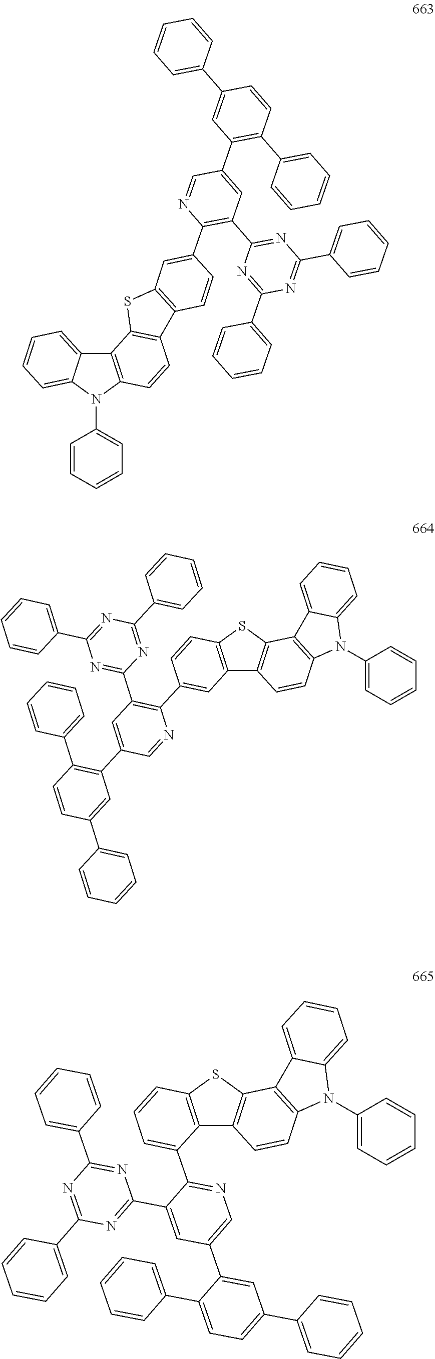 [SMILES: c1ccc(-c2ccc(-c3ccccc3)c(-c3cnc(-c4ccc5c(c4)sc4c5ccc5c4c4ccccc4n5-c4ccccc4)c(-c4nc(-c5ccccc5)nc(-c5ccccc5)n4)c3)c2)cc1 ; c1ccc(-c2ccc(-c3ccccc3)c(-c3cnc(-c4ccc5sc6c(ccc7c6c6ccccc6n7-c6ccccc6)c5c4)c(-c4nc(-c5ccccc5)nc(-c5ccccc5)n4)c3)c2)cc1 ; c1ccc(-c2ccc(-c3ccccc3)c(-c3cnc(-c4cccc5sc6c(ccc7c6c6ccccc6n7-c6ccccc6)c45)c(-c4nc(-c5ccccc5)nc(-c5ccccc5)n4)c3)c2)cc1]

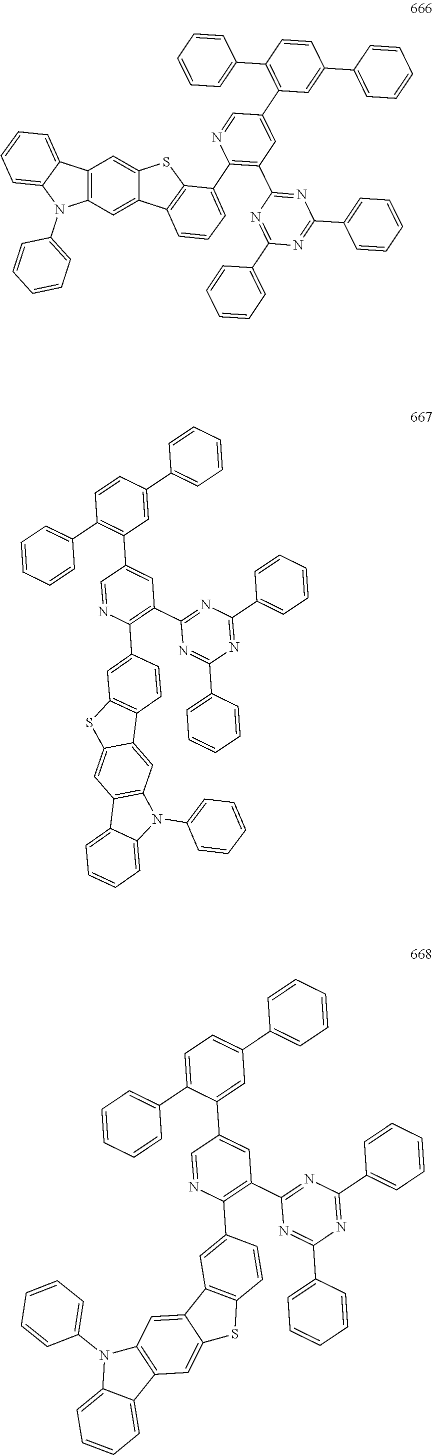 [SMILES: c1ccc(-c2ccc(-c3ccccc3)c(-c3cnc(-c4cccc5c4sc4cc6c7ccccc7n(-c7ccccc7)c6cc45)c(-c4nc(-c5ccccc5)nc(-c5ccccc5)n4)c3)c2)cc1 ; c1ccc(-c2ccc(-c3ccccc3)c(-c3cnc(-c4ccc5c(c4)sc4cc6c7ccccc7n(-c7ccccc7)c6cc45)c(-c4nc(-c5ccccc5)nc(-c5ccccc5)n4)c3)c2)cc1 ; c1ccc(-c2ccc(-c3ccccc3)c(-c3cnc(-c4ccc5sc6cc7c8ccccc8n(-c8ccccc8)c7cc6c5c4)c(-c4nc(-c5ccccc5)nc(-c5ccccc5)n4)c3)c2)cc1]

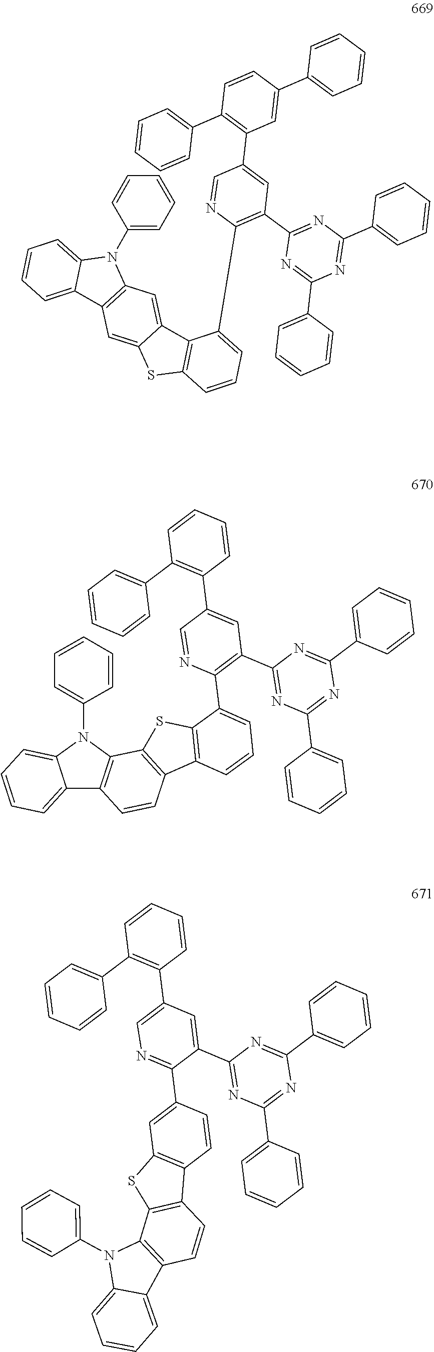 [SMILES: c1ccc(-c2ccc(-c3ccccc3)c(-c3cnc(-c4cccc5sc6cc7c8ccccc8n(-c8ccccc8)c7cc6c45)c(-c4nc(-c5ccccc5)nc(-c5ccccc5)n4)c3)c2)cc1 ; c1ccc(-c2nc(-c3ccccc3)nc(-c3cc(-c4ccccc4-c4ccccc4)cnc3-c3cccc4c3sc3c4ccc4c5ccccc5n(-c5ccccc5)c43)n2)cc1 ; c1ccc(-c2nc(-c3ccccc3)nc(-c3cc(-c4ccccc4-c4ccccc4)cnc3-c3ccc4c(c3)sc3c4ccc4c5ccccc5n(-c5ccccc5)c43)n2)cc1]

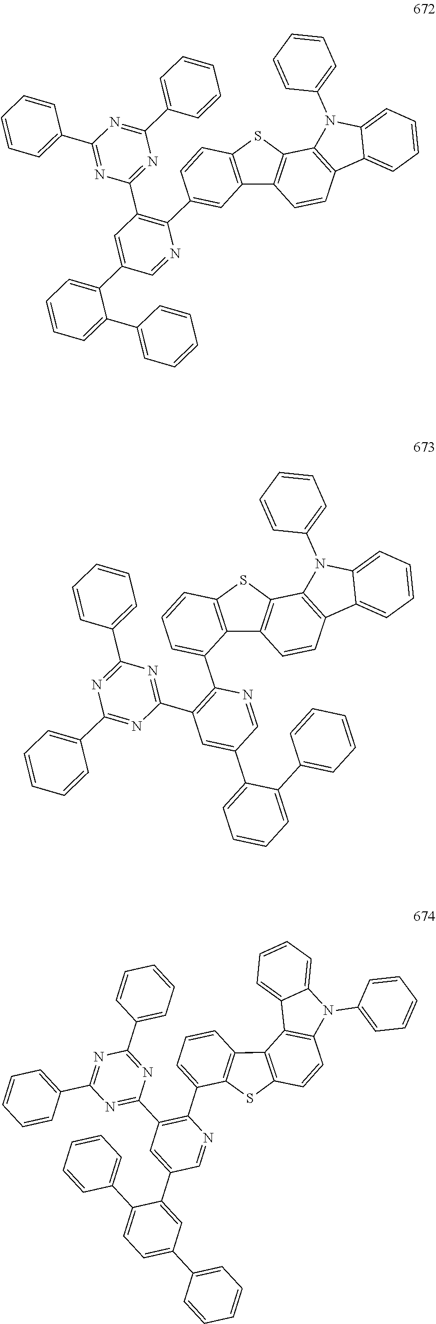 [SMILES: c1ccc(-c2nc(-c3ccccc3)nc(-c3cc(-c4ccccc4-c4ccccc4)cnc3-c3ccc4sc5c(ccc6c7ccccc7n(-c7ccccc7)c65)c4c3)n2)cc1 ; c1ccc(-c2nc(-c3ccccc3)nc(-c3cc(-c4ccccc4-c4ccccc4)cnc3-c3cccc4sc5c(ccc6c7ccccc7n(-c7ccccc7)c65)c34)n2)cc1 ; c1ccc(-c2ccc(-c3ccccc3)c(-c3cnc(-c4cccc5c4sc4ccc6c(c7ccccc7n6-c6ccccc6)c45)c(-c4nc(-c5ccccc5)nc(-c5ccccc5)n4)c3)c2)cc1]

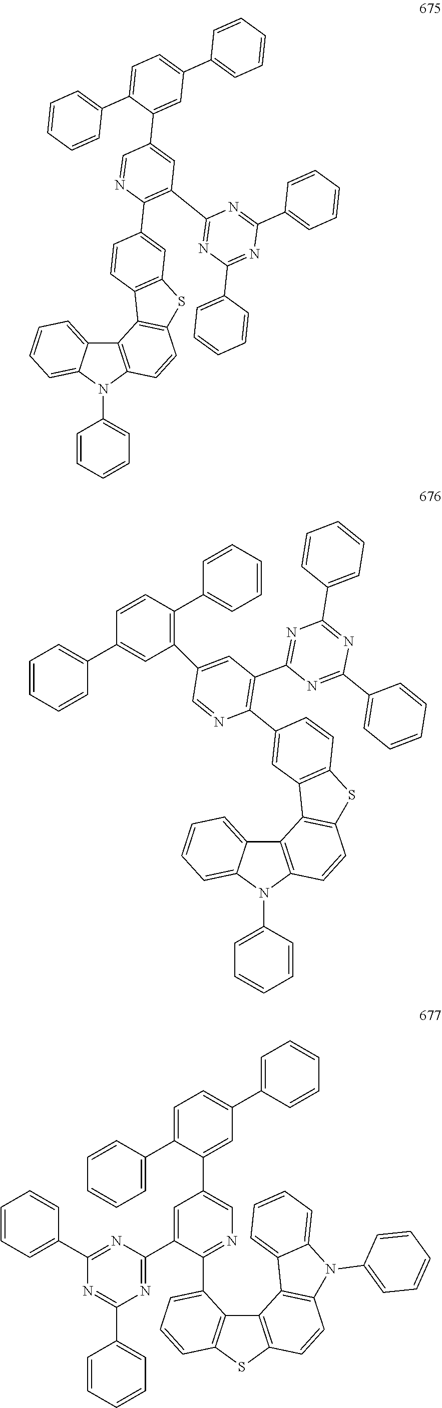 [SMILES: c1ccc(-c2ccc(-c3ccccc3)c(-c3cnc(-c4ccc5c(c4)sc4ccc6c(c7ccccc7n6-c6ccccc6)c45)c(-c4nc(-c5ccccc5)nc(-c5ccccc5)n4)c3)c2)cc1 ; c1ccc(-c2ccc(-c3ccccc3)c(-c3cnc(-c4ccc5sc6ccc7c(c8ccccc8n7-c7ccccc7)c6c5c4)c(-c4nc(-c5ccccc5)nc(-c5ccccc5)n4)c3)c2)cc1 ; c1ccc(-c2ccc(-c3ccccc3)c(-c3cnc(-c4cccc5sc6ccc7c(c8ccccc8n7-c7ccccc7)c6c45)c(-c4nc(-c5ccccc5)nc(-c5ccccc5)n4)c3)c2)cc1]

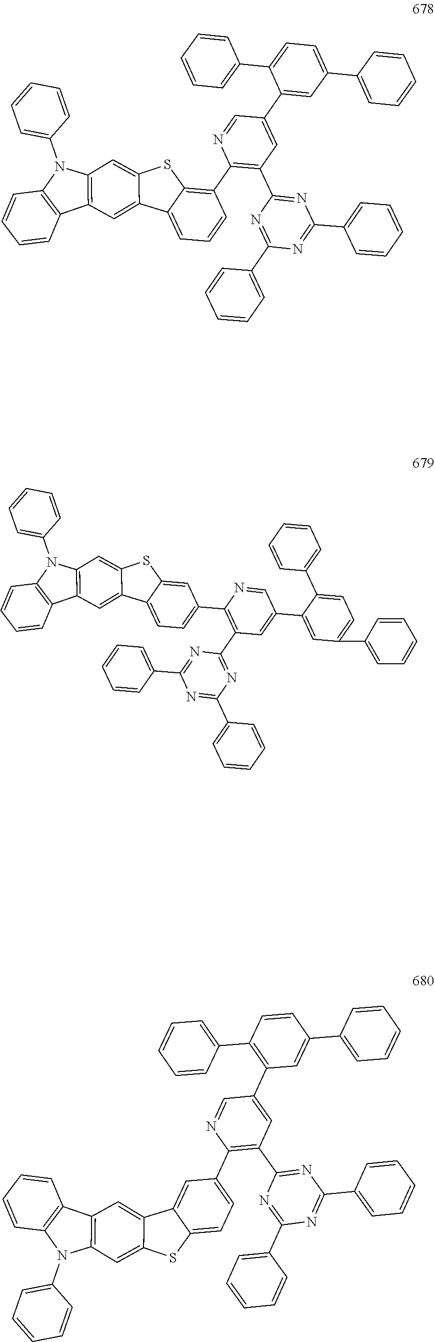 [SMILES: c1ccc(-c2ccc(-c3ccccc3)c(-c3cnc(-c4cccc5c4sc4cc6c(cc45)c4ccccc4n6-c4ccccc4)c(-c4nc(-c5ccccc5)nc(-c5ccccc5)n4)c3)c2)cc1 ; c1ccc(-c2ccc(-c3ccccc3)c(-c3cnc(-c4ccc5c(c4)sc4cc6c(cc45)c4ccccc4n6-c4ccccc4)c(-c4nc(-c5ccccc5)nc(-c5ccccc5)n4)c3)c2)cc1 ; c1ccc(-c2ccc(-c3ccccc3)c(-c3cnc(-c4ccc5sc6cc7c(cc6c5c4)c4ccccc4n7-c4ccccc4)c(-c4nc(-c5ccccc5)nc(-c5ccccc5)n4)c3)c2)cc1]

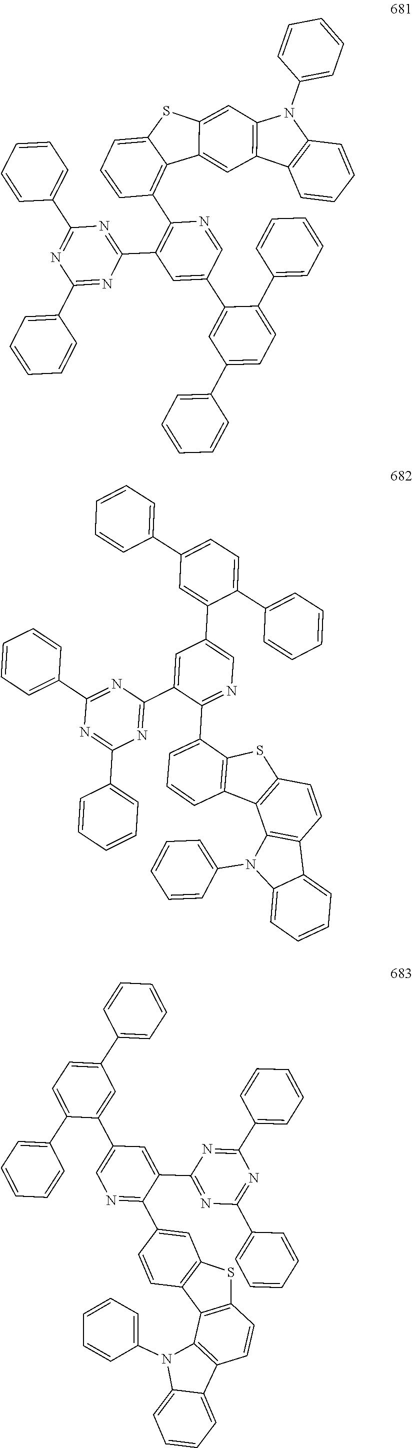 [SMILES: c1ccc(-c2ccc(-c3ccccc3)c(-c3cnc(-c4cccc5sc6cc7c(cc6c45)c4ccccc4n7-c4ccccc4)c(-c4nc(-c5ccccc5)nc(-c5ccccc5)n4)c3)c2)cc1 ; c1ccc(-c2ccc(-c3ccccc3)c(-c3cnc(-c4cccc5c4sc4ccc6c7ccccc7n(-c7ccccc7)c6c45)c(-c4nc(-c5ccccc5)nc(-c5ccccc5)n4)c3)c2)cc1 ; c1ccc(-c2ccc(-c3ccccc3)c(-c3cnc(-c4ccc5c(c4)sc4ccc6c7ccccc7n(-c7ccccc7)c6c45)c(-c4nc(-c5ccccc5)nc(-c5ccccc5)n4)c3)c2)cc1]

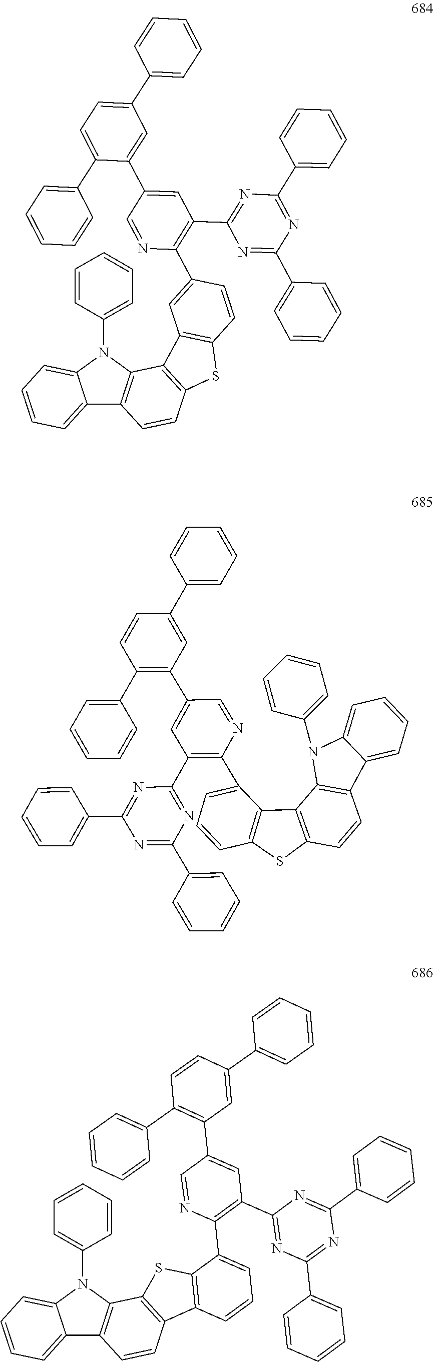 [SMILES: c1ccc(-c2ccc(-c3ccccc3)c(-c3cnc(-c4ccc5sc6ccc7c8ccccc8n(-c8ccccc8)c7c6c5c4)c(-c4nc(-c5ccccc5)nc(-c5ccccc5)n4)c3)c2)cc1 ; c1ccc(-c2ccc(-c3ccccc3)c(-c3cnc(-c4cccc5sc6ccc7c8ccccc8n(-c8ccccc8)c7c6c45)c(-c4nc(-c5ccccc5)nc(-c5ccccc5)n4)c3)c2)cc1 ; c1ccc(-c2ccc(-c3ccccc3)c(-c3cnc(-c4cccc5c4sc4c5ccc5c6ccccc6n(-c6ccccc6)c54)c(-c4nc(-c5ccccc5)nc(-c5ccccc5)n4)c3)c2)cc1]

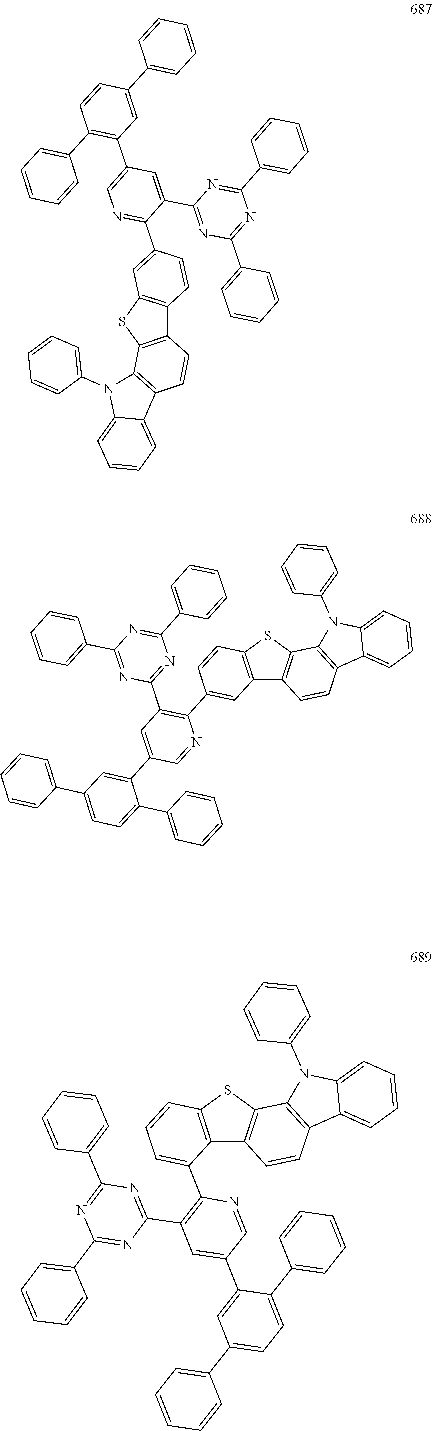 [SMILES: c1ccc(-c2ccc(-c3ccccc3)c(-c3cnc(-c4ccc5c(c4)sc4c5ccc5c6ccccc6n(-c6ccccc6)c54)c(-c4nc(-c5ccccc5)nc(-c5ccccc5)n4)c3)c2)cc1 ; c1ccc(-c2ccc(-c3ccccc3)c(-c3cnc(-c4ccc5sc6c(ccc7c8ccccc8n(-c8ccccc8)c76)c5c4)c(-c4nc(-c5ccccc5)nc(-c5ccccc5)n4)c3)c2)cc1 ; c1ccc(-c2ccc(-c3ccccc3)c(-c3cnc(-c4cccc5sc6c(ccc7c8ccccc8n(-c8ccccc8)c76)c45)c(-c4nc(-c5ccccc5)nc(-c5ccccc5)n4)c3)c2)cc1]

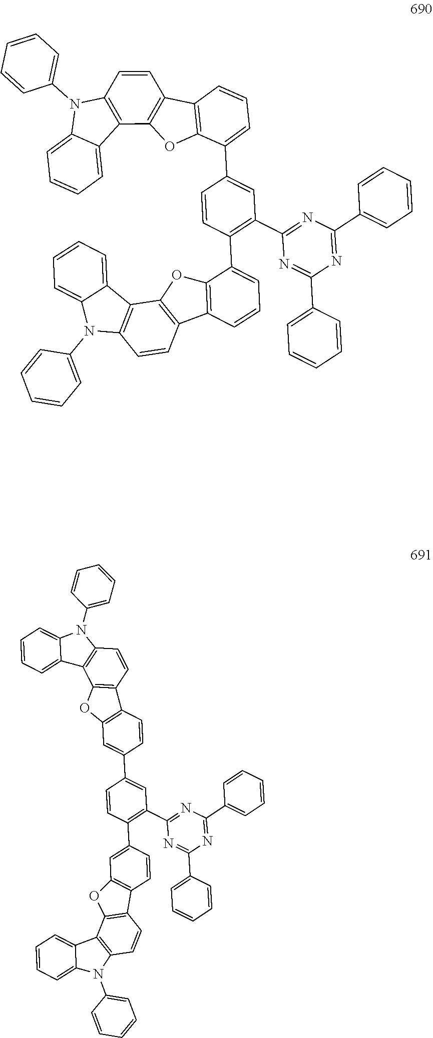 [SMILES: c1ccc(-c2nc(-c3ccccc3)nc(-c3cc(-c4cccc5c4oc4c5ccc5c4c4ccccc4n5-c4ccccc4)ccc3-c3cccc4c3oc3c4ccc4c3c3ccccc3n4-c3ccccc3)n2)cc1 ; c1ccc(-c2nc(-c3ccccc3)nc(-c3cc(-c4ccc5c(c4)oc4c5ccc5c4c4ccccc4n5-c4ccccc4)ccc3-c3ccc4c(c3)oc3c4ccc4c3c3ccccc3n4-c3ccccc3)n2)cc1]

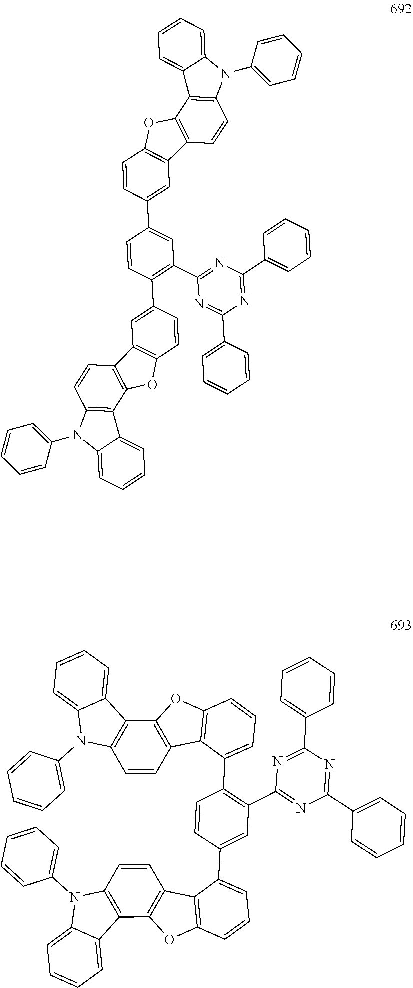 [SMILES: c1ccc(-c2nc(-c3ccccc3)nc(-c3cc(-c4ccc5oc6c(ccc7c6c6ccccc6n7-c6ccccc6)c5c4)ccc3-c3ccc4oc5c(ccc6c5c5ccccc5n6-c5ccccc5)c4c3)n2)cc1 ; c1ccc(-c2nc(-c3ccccc3)nc(-c3cc(-c4cccc5oc6c(ccc7c6c6ccccc6n7-c6ccccc6)c45)ccc3-c3cccc4oc5c(ccc6c5c5ccccc5n6-c5ccccc5)c34)n2)cc1]

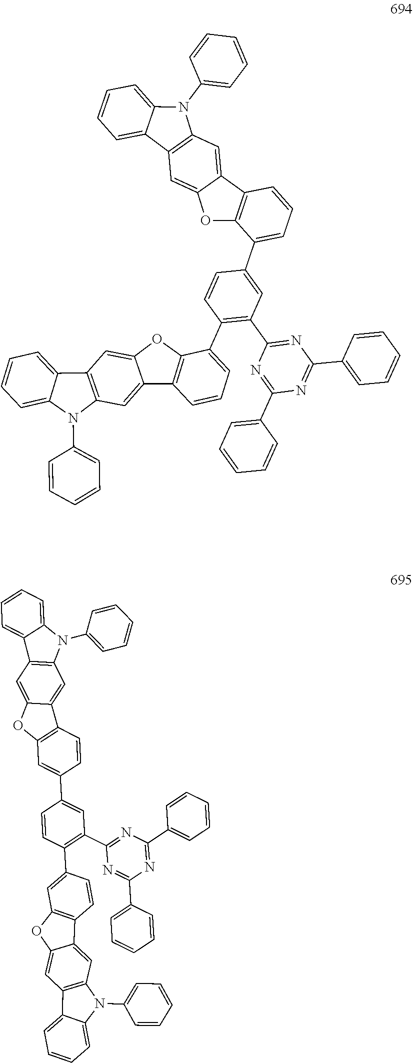 [SMILES: c1ccc(-c2nc(-c3ccccc3)nc(-c3cc(-c4cccc5c4oc4cc6c7ccccc7n(-c7ccccc7)c6cc45)ccc3-c3cccc4c3oc3cc5c6ccccc6n(-c6ccccc6)c5cc34)n2)cc1 ; c1ccc(-c2nc(-c3ccccc3)nc(-c3cc(-c4ccc5c(c4)oc4cc6c7ccccc7n(-c7ccccc7)c6cc45)ccc3-c3ccc4c(c3)oc3cc5c6ccccc6n(-c6ccccc6)c5cc34)n2)cc1]

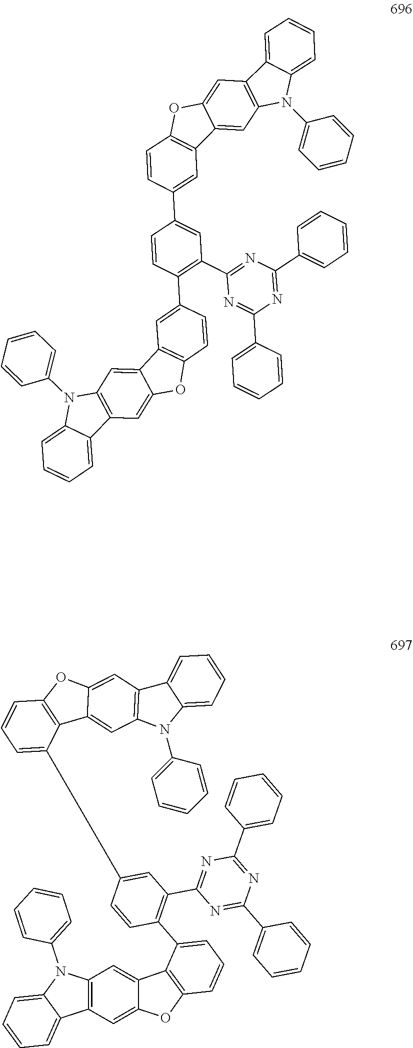 [SMILES: c1ccc(-c2nc(-c3ccccc3)nc(-c3cc(-c4ccc5oc6cc7c8ccccc8n(-c8ccccc8)c7cc6c5c4)ccc3-c3ccc4oc5cc6c7ccccc7n(-c7ccccc7)c6cc5c4c3)n2)cc1 ; c1ccc(-c2nc(-c3ccccc3)nc(-c3cc(-c4cccc5oc6cc7c8ccccc8n(-c8ccccc8)c7cc6c45)ccc3-c3cccc4oc5cc6c7ccccc7n(-c7ccccc7)c6cc5c34)n2)cc1]

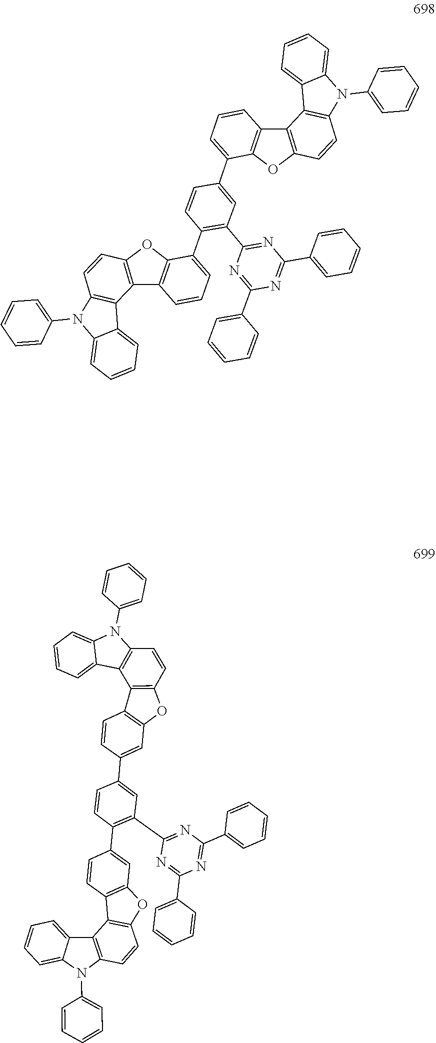 [SMILES: c1ccc(-c2nc(-c3ccccc3)nc(-c3cc(-c4cccc5c4oc4ccc6c(c7ccccc7n6-c6ccccc6)c45)ccc3-c3cccc4c3oc3ccc5c(c6ccccc6n5-c5ccccc5)c34)n2)cc1 ; c1ccc(-c2nc(-c3ccccc3)nc(-c3cc(-c4ccc5c(c4)oc4ccc6c(c7ccccc7n6-c6ccccc6)c45)ccc3-c3ccc4c(c3)oc3ccc5c(c6ccccc6n5-c5ccccc5)c34)n2)cc1]

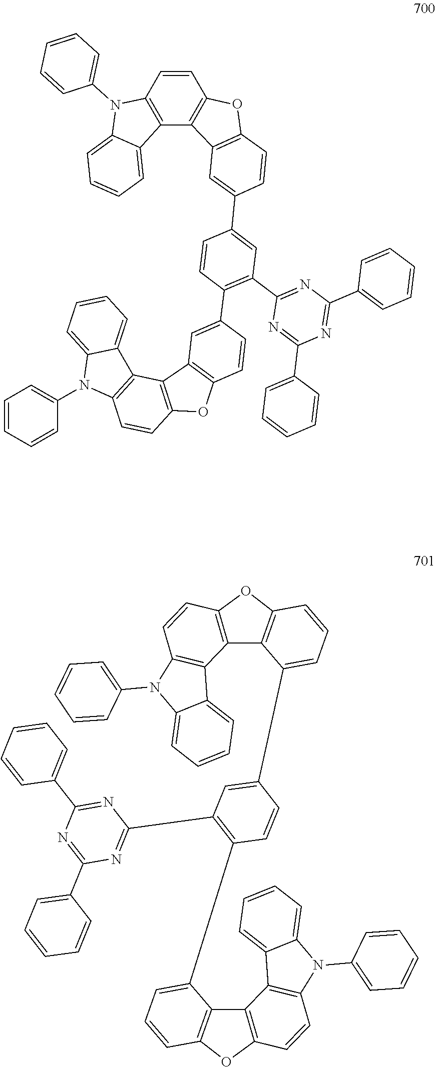 [SMILES: c1ccc(-c2nc(-c3ccccc3)nc(-c3cc(-c4ccc5oc6ccc7c(c8ccccc8n7-c7ccccc7)c6c5c4)ccc3-c3ccc4oc5ccc6c(c7ccccc7n6-c6ccccc6)c5c4c3)n2)cc1 ; c1ccc(-c2nc(-c3ccccc3)nc(-c3cc(-c4cccc5oc6ccc7c(c8ccccc8n7-c7ccccc7)c6c45)ccc3-c3cccc4oc5ccc6c(c7ccccc7n6-c6ccccc6)c5c34)n2)cc1]

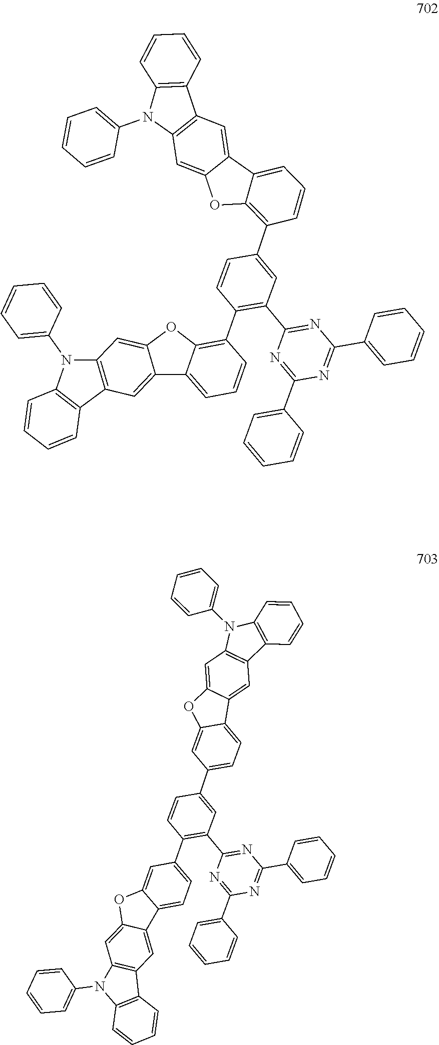 [SMILES: c1ccc(-c2nc(-c3ccccc3)nc(-c3cc(-c4cccc5c4oc4cc6c(cc45)c4ccccc4n6-c4ccccc4)ccc3-c3cccc4c3oc3cc5c(cc34)c3ccccc3n5-c3ccccc3)n2)cc1 ; c1ccc(-c2nc(-c3ccccc3)nc(-c3cc(-c4ccc5c(c4)oc4cc6c(cc45)c4ccccc4n6-c4ccccc4)ccc3-c3ccc4c(c3)oc3cc5c(cc34)c3ccccc3n5-c3ccccc3)n2)cc1]

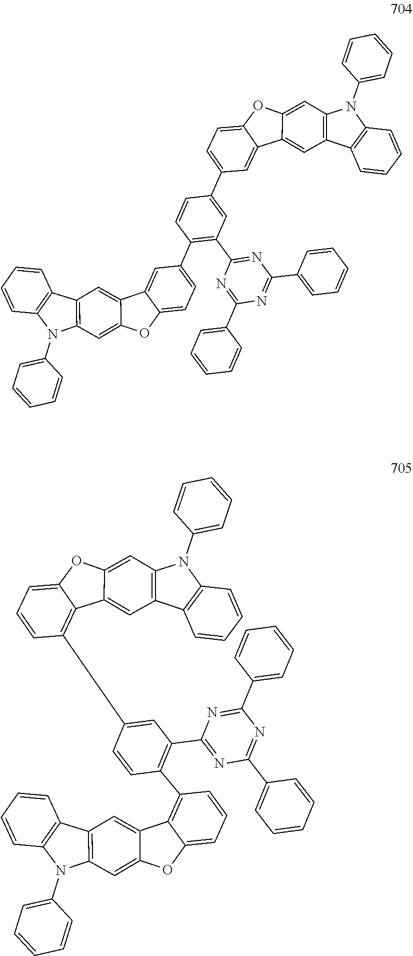 [SMILES: c1ccc(-c2nc(-c3ccccc3)nc(-c3cc(-c4ccc5oc6cc7c(cc6c5c4)c4ccccc4n7-c4ccccc4)ccc3-c3ccc4oc5cc6c(cc5c4c3)c3ccccc3n6-c3ccccc3)n2)cc1 ; c1ccc(-c2nc(-c3ccccc3)nc(-c3cc(-c4cccc5oc6cc7c(cc6c45)c4ccccc4n7-c4ccccc4)ccc3-c3cccc4oc5cc6c(cc5c34)c3ccccc3n6-c3ccccc3)n2)cc1]

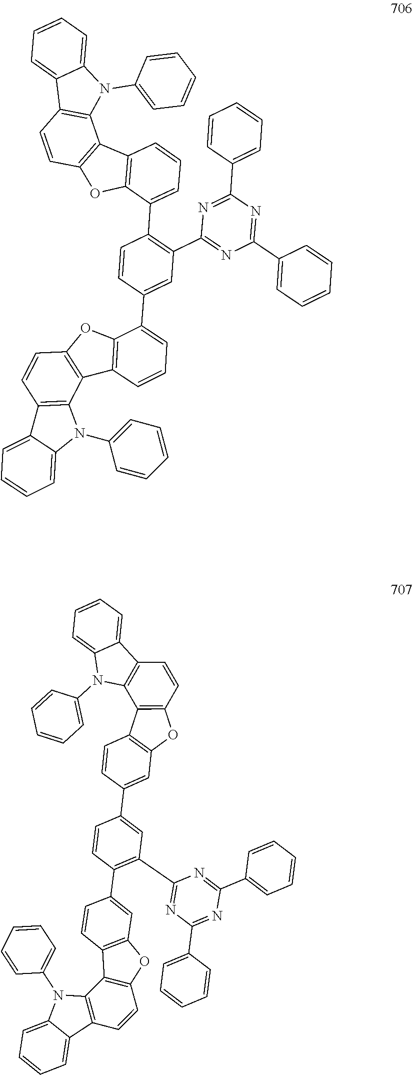 [SMILES: c1ccc(-c2nc(-c3ccccc3)nc(-c3cc(-c4cccc5c4oc4ccc6c7ccccc7n(-c7ccccc7)c6c45)ccc3-c3cccc4c3oc3ccc5c6ccccc6n(-c6ccccc6)c5c34)n2)cc1 ; c1ccc(-c2nc(-c3ccccc3)nc(-c3cc(-c4ccc5c(c4)oc4ccc6c7ccccc7n(-c7ccccc7)c6c45)ccc3-c3ccc4c(c3)oc3ccc5c6ccccc6n(-c6ccccc6)c5c34)n2)cc1]

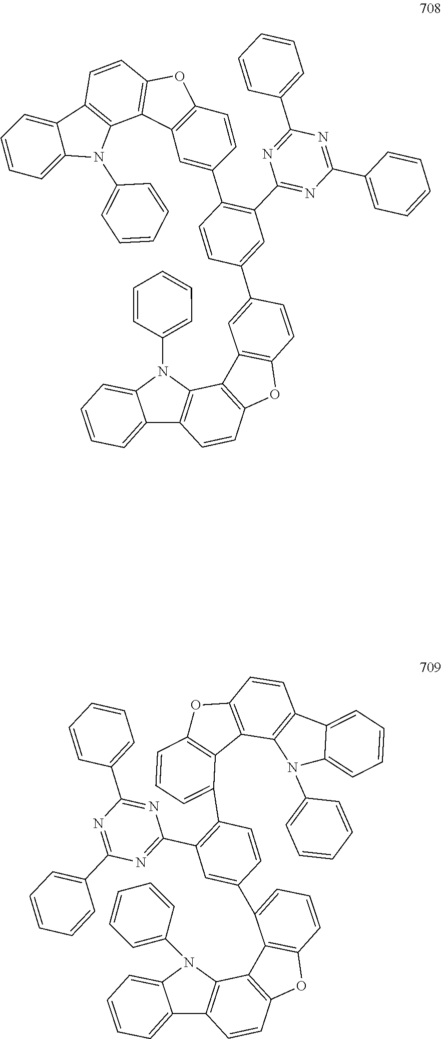 [SMILES: c1ccc(-c2nc(-c3ccccc3)nc(-c3cc(-c4ccc5oc6ccc7c8ccccc8n(-c8ccccc8)c7c6c5c4)ccc3-c3ccc4oc5ccc6c7ccccc7n(-c7ccccc7)c6c5c4c3)n2)cc1 ; c1ccc(-c2nc(-c3ccccc3)nc(-c3cc(-c4cccc5oc6ccc7c8ccccc8n(-c8ccccc8)c7c6c45)ccc3-c3cccc4oc5ccc6c7ccccc7n(-c7ccccc7)c6c5c34)n2)cc1]

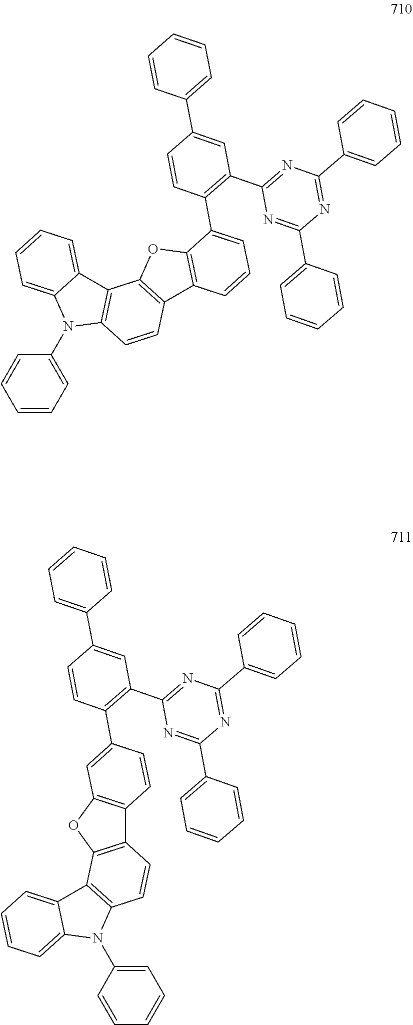 [SMILES: c1ccc(-c2ccc(-c3cccc4c3oc3c4ccc4c3c3ccccc3n4-c3ccccc3)c(-c3nc(-c4ccccc4)nc(-c4ccccc4)n3)c2)cc1 ; c1ccc(-c2ccc(-c3ccc4c(c3)oc3c4ccc4c3c3ccccc3n4-c3ccccc3)c(-c3nc(-c4ccccc4)nc(-c4ccccc4)n3)c2)cc1]

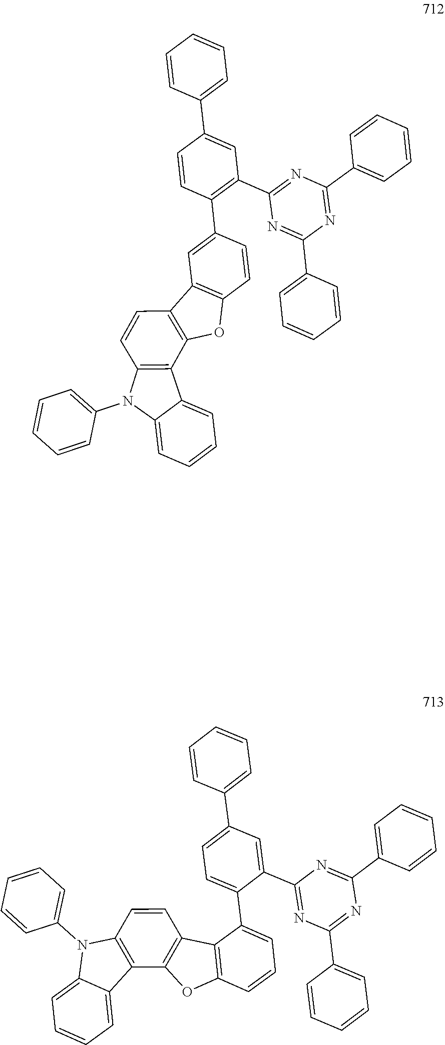 [SMILES: c1ccc(-c2ccc(-c3ccc4oc5c(ccc6c5c5ccccc5n6-c5ccccc5)c4c3)c(-c3nc(-c4ccccc4)nc(-c4ccccc4)n3)c2)cc1 ; c1ccc(-c2ccc(-c3cccc4oc5c(ccc6c5c5ccccc5n6-c5ccccc5)c34)c(-c3nc(-c4ccccc4)nc(-c4ccccc4)n3)c2)cc1]

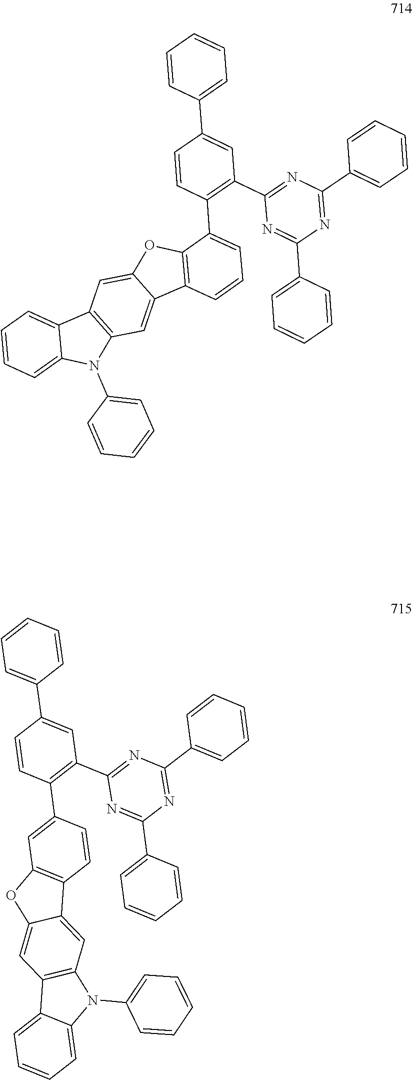 [SMILES: c1ccc(-c2ccc(-c3cccc4c3oc3cc5c6ccccc6n(-c6ccccc6)c5cc34)c(-c3nc(-c4ccccc4)nc(-c4ccccc4)n3)c2)cc1 ; c1ccc(-c2ccc(-c3ccc4c(c3)oc3cc5c6ccccc6n(-c6ccccc6)c5cc34)c(-c3nc(-c4ccccc4)nc(-c4ccccc4)n3)c2)cc1]

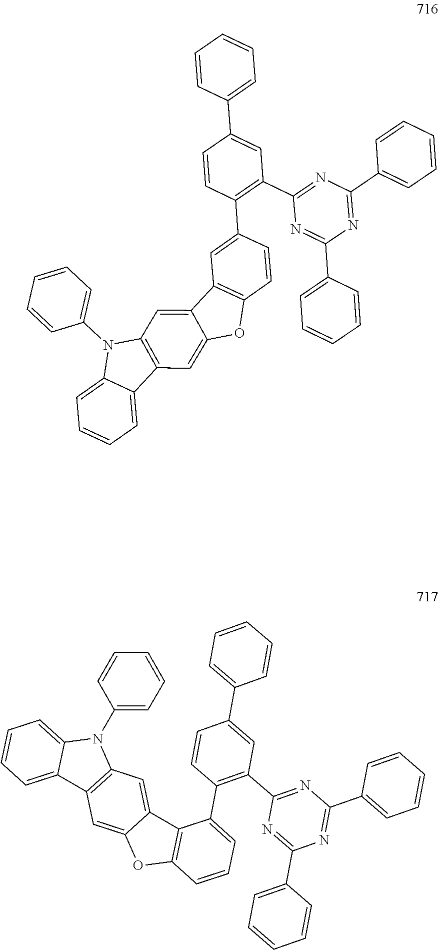 [SMILES: c1ccc(-c2ccc(-c3ccc4oc5cc6c7ccccc7n(-c7ccccc7)c6cc5c4c3)c(-c3nc(-c4ccccc4)nc(-c4ccccc4)n3)c2)cc1 ; c1ccc(-c2ccc(-c3cccc4oc5cc6c7ccccc7n(-c7ccccc7)c6cc5c34)c(-c3nc(-c4ccccc4)nc(-c4ccccc4)n3)c2)cc1]

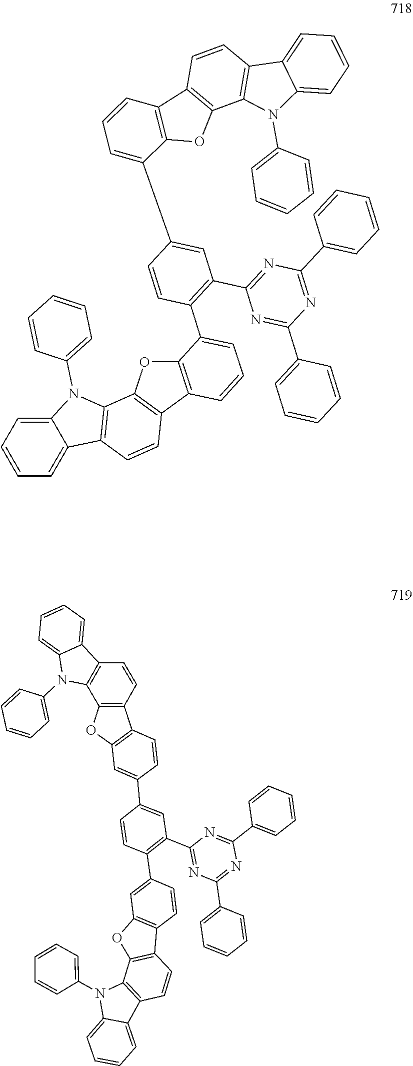 [SMILES: c1ccc(-c2nc(-c3ccccc3)nc(-c3cc(-c4cccc5c4oc4c5ccc5c6ccccc6n(-c6ccccc6)c54)ccc3-c3cccc4c3oc3c4ccc4c5ccccc5n(-c5ccccc5)c43)n2)cc1 ; c1ccc(-c2nc(-c3ccccc3)nc(-c3cc(-c4ccc5c(c4)oc4c5ccc5c6ccccc6n(-c6ccccc6)c54)ccc3-c3ccc4c(c3)oc3c4ccc4c5ccccc5n(-c5ccccc5)c43)n2)cc1]

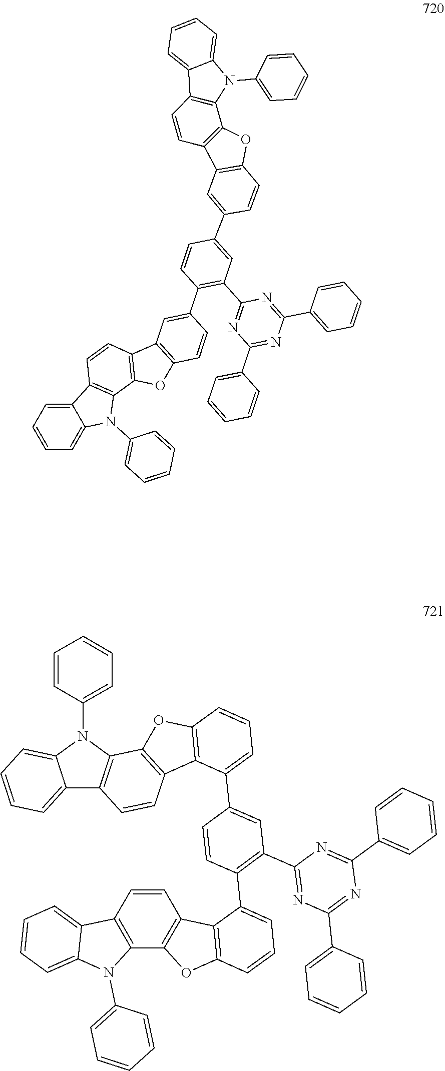 [SMILES: c1ccc(-c2nc(-c3ccccc3)nc(-c3cc(-c4ccc5oc6c(ccc7c8ccccc8n(-c8ccccc8)c76)c5c4)ccc3-c3ccc4oc5c(ccc6c7ccccc7n(-c7ccccc7)c65)c4c3)n2)cc1 ; c1ccc(-c2nc(-c3ccccc3)nc(-c3cc(-c4cccc5oc6c(ccc7c8ccccc8n(-c8ccccc8)c76)c45)ccc3-c3cccc4oc5c(ccc6c7ccccc7n(-c7ccccc7)c65)c34)n2)cc1]

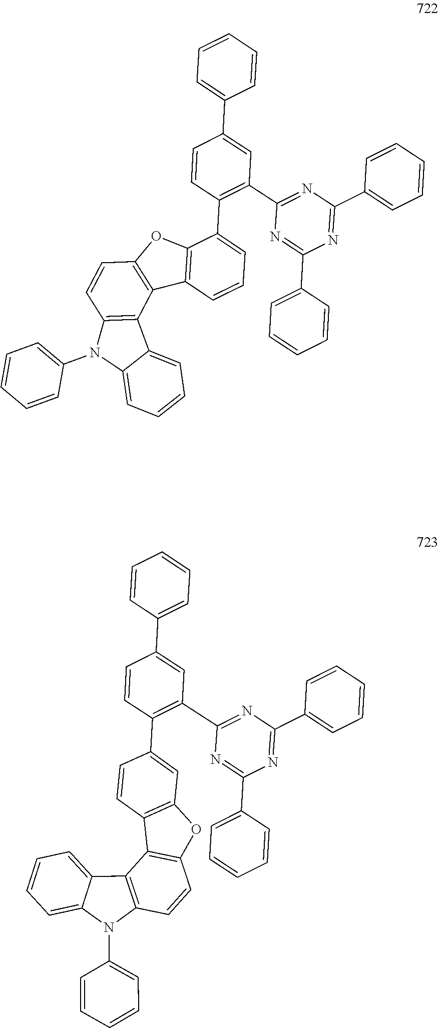 [SMILES: c1ccc(-c2ccc(-c3cccc4c3oc3ccc5c(c6ccccc6n5-c5ccccc5)c34)c(-c3nc(-c4ccccc4)nc(-c4ccccc4)n3)c2)cc1 ; c1ccc(-c2ccc(-c3ccc4c(c3)oc3ccc5c(c6ccccc6n5-c5ccccc5)c34)c(-c3nc(-c4ccccc4)nc(-c4ccccc4)n3)c2)cc1]

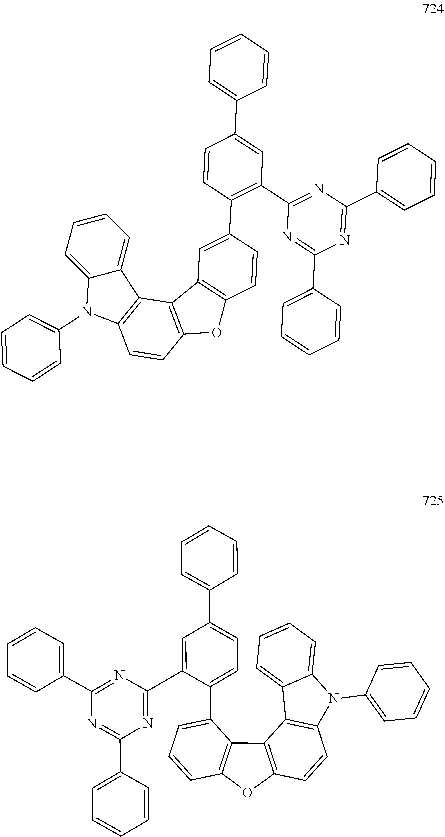 [SMILES: c1ccc(-c2ccc(-c3ccc4oc5ccc6c(c7ccccc7n6-c6ccccc6)c5c4c3)c(-c3nc(-c4ccccc4)nc(-c4ccccc4)n3)c2)cc1 ; c1ccc(-c2ccc(-c3cccc4oc5ccc6c(c7ccccc7n6-c6ccccc6)c5c34)c(-c3nc(-c4ccccc4)nc(-c4ccccc4)n3)c2)cc1]

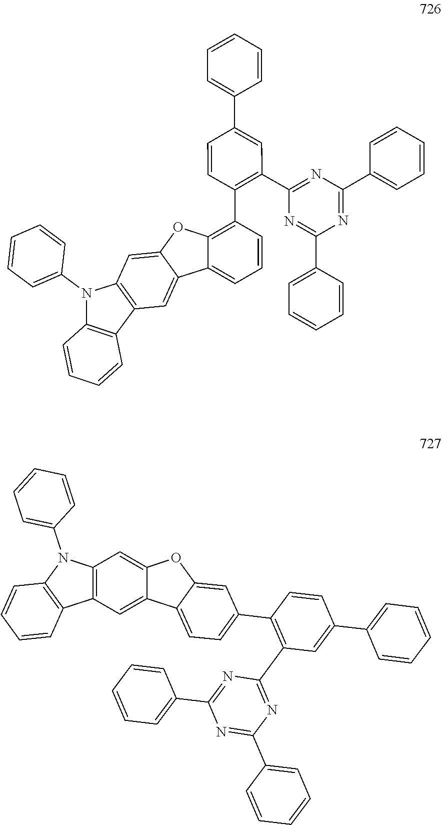 [SMILES: c1ccc(-c2ccc(-c3cccc4c3oc3cc5c(cc34)c3ccccc3n5-c3ccccc3)c(-c3nc(-c4ccccc4)nc(-c4ccccc4)n3)c2)cc1 ; c1ccc(-c2ccc(-c3ccc4c(c3)oc3cc5c(cc34)c3ccccc3n5-c3ccccc3)c(-c3nc(-c4ccccc4)nc(-c4ccccc4)n3)c2)cc1]

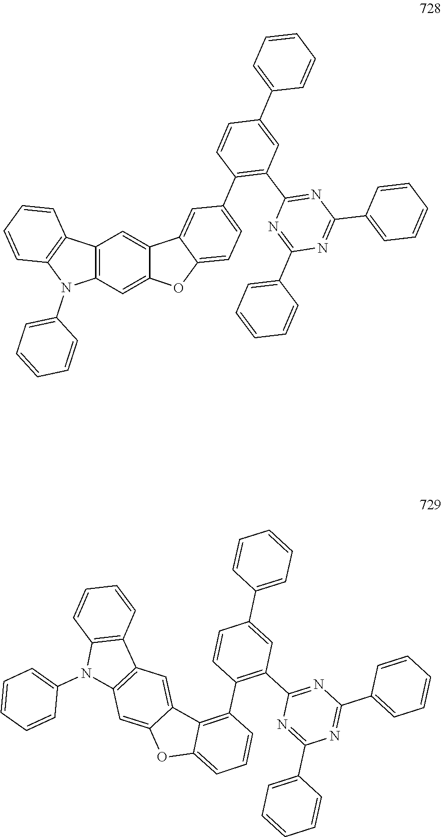 [SMILES: c1ccc(-c2ccc(-c3ccc4oc5cc6c(cc5c4c3)c3ccccc3n6-c3ccccc3)c(-c3nc(-c4ccccc4)nc(-c4ccccc4)n3)c2)cc1 ; c1ccc(-c2ccc(-c3cccc4oc5cc6c(cc5c34)c3ccccc3n6-c3ccccc3)c(-c3nc(-c4ccccc4)nc(-c4ccccc4)n3)c2)cc1]

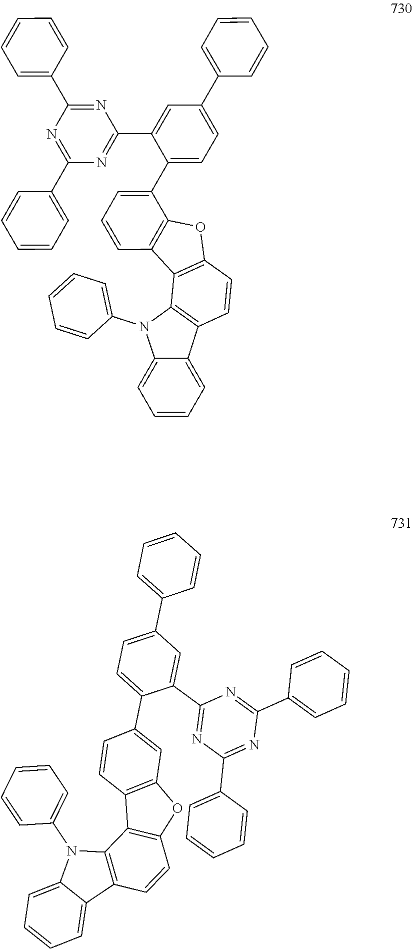 [SMILES: c1ccc(-c2ccc(-c3cccc4c3oc3ccc5c6ccccc6n(-c6ccccc6)c5c34)c(-c3nc(-c4ccccc4)nc(-c4ccccc4)n3)c2)cc1 ; c1ccc(-c2ccc(-c3ccc4c(c3)oc3ccc5c6ccccc6n(-c6ccccc6)c5c34)c(-c3nc(-c4ccccc4)nc(-c4ccccc4)n3)c2)cc1]

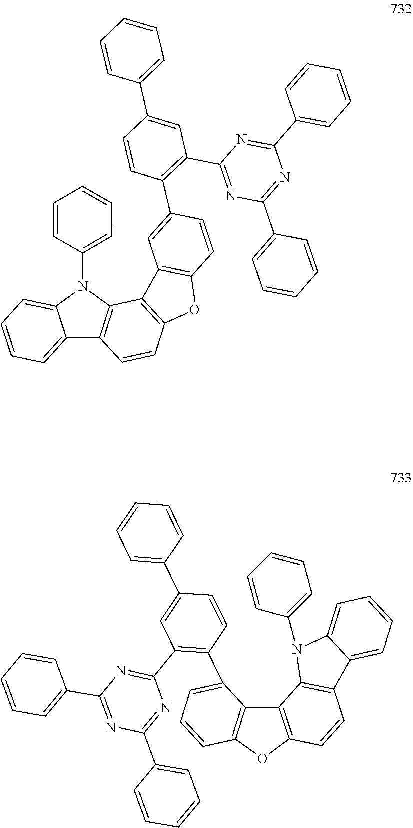 [SMILES: c1ccc(-c2ccc(-c3ccc4oc5ccc6c7ccccc7n(-c7ccccc7)c6c5c4c3)c(-c3nc(-c4ccccc4)nc(-c4ccccc4)n3)c2)cc1 ; c1ccc(-c2ccc(-c3cccc4oc5ccc6c7ccccc7n(-c7ccccc7)c6c5c34)c(-c3nc(-c4ccccc4)nc(-c4ccccc4)n3)c2)cc1]

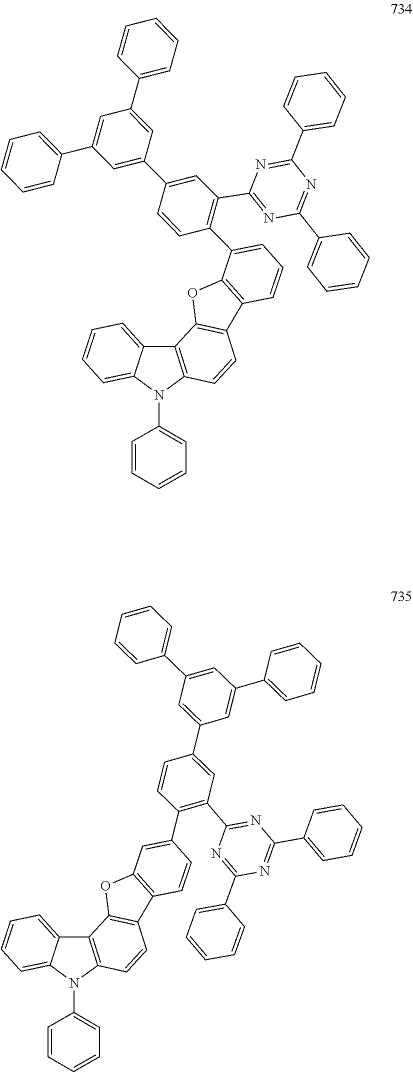 [SMILES: c1ccc(-c2cc(-c3ccccc3)cc(-c3ccc(-c4cccc5c4oc4c5ccc5c4c4ccccc4n5-c4ccccc4)c(-c4nc(-c5ccccc5)nc(-c5ccccc5)n4)c3)c2)cc1 ; c1ccc(-c2cc(-c3ccccc3)cc(-c3ccc(-c4ccc5c(c4)oc4c5ccc5c4c4ccccc4n5-c4ccccc4)c(-c4nc(-c5ccccc5)nc(-c5ccccc5)n4)c3)c2)cc1]

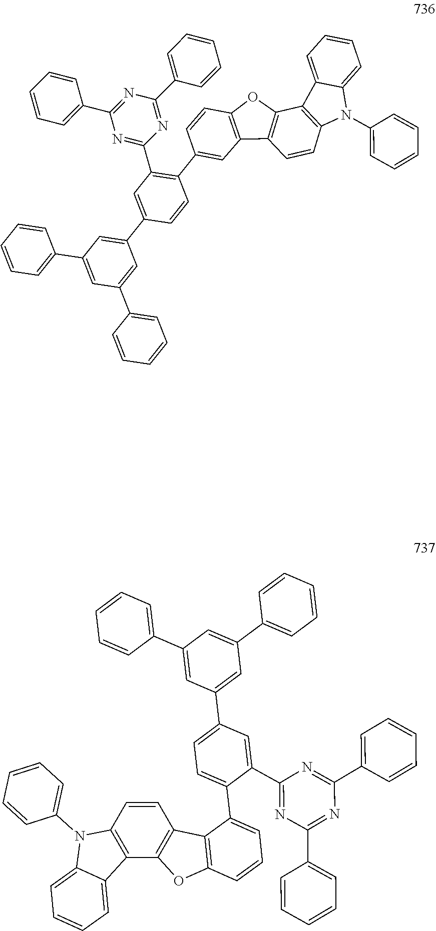 [SMILES: c1ccc(-c2cc(-c3ccccc3)cc(-c3ccc(-c4ccc5oc6c(ccc7c6c6ccccc6n7-c6ccccc6)c5c4)c(-c4nc(-c5ccccc5)nc(-c5ccccc5)n4)c3)c2)cc1 ; c1ccc(-c2cc(-c3ccccc3)cc(-c3ccc(-c4cccc5oc6c(ccc7c6c6ccccc6n7-c6ccccc6)c45)c(-c4nc(-c5ccccc5)nc(-c5ccccc5)n4)c3)c2)cc1]

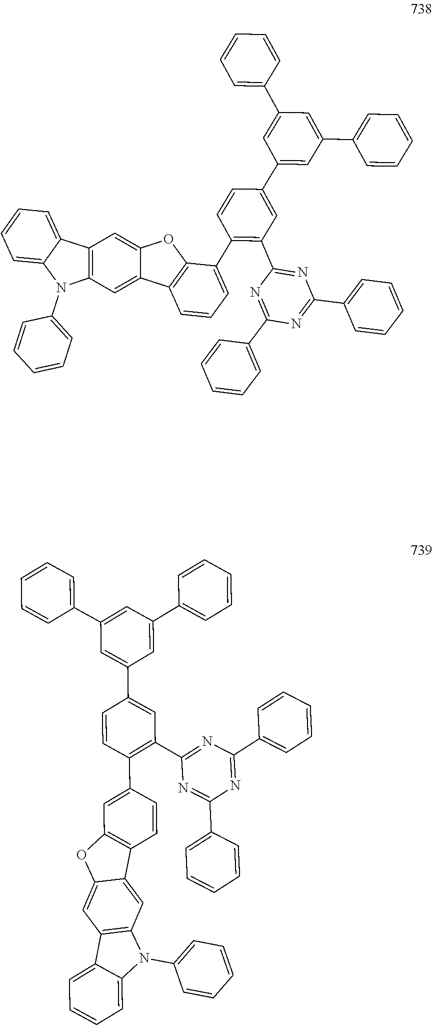 [SMILES: c1ccc(-c2cc(-c3ccccc3)cc(-c3ccc(-c4cccc5c4oc4cc6c7ccccc7n(-c7ccccc7)c6cc45)c(-c4nc(-c5ccccc5)nc(-c5ccccc5)n4)c3)c2)cc1 ; c1ccc(-c2cc(-c3ccccc3)cc(-c3ccc(-c4ccc5c(c4)oc4cc6c7ccccc7n(-c7ccccc7)c6cc45)c(-c4nc(-c5ccccc5)nc(-c5ccccc5)n4)c3)c2)cc1]

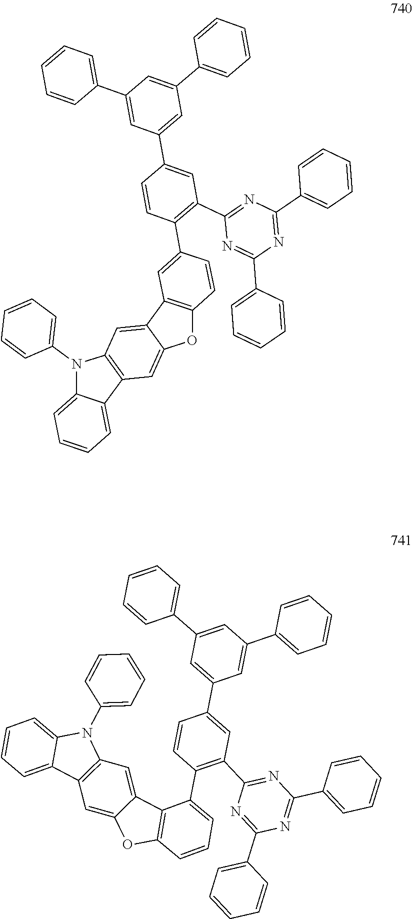 [SMILES: c1ccc(-c2cc(-c3ccccc3)cc(-c3ccc(-c4ccc5oc6cc7c8ccccc8n(-c8ccccc8)c7cc6c5c4)c(-c4nc(-c5ccccc5)nc(-c5ccccc5)n4)c3)c2)cc1 ; c1ccc(-c2cc(-c3ccccc3)cc(-c3ccc(-c4cccc5oc6cc7c8ccccc8n(-c8ccccc8)c7cc6c45)c(-c4nc(-c5ccccc5)nc(-c5ccccc5)n4)c3)c2)cc1]

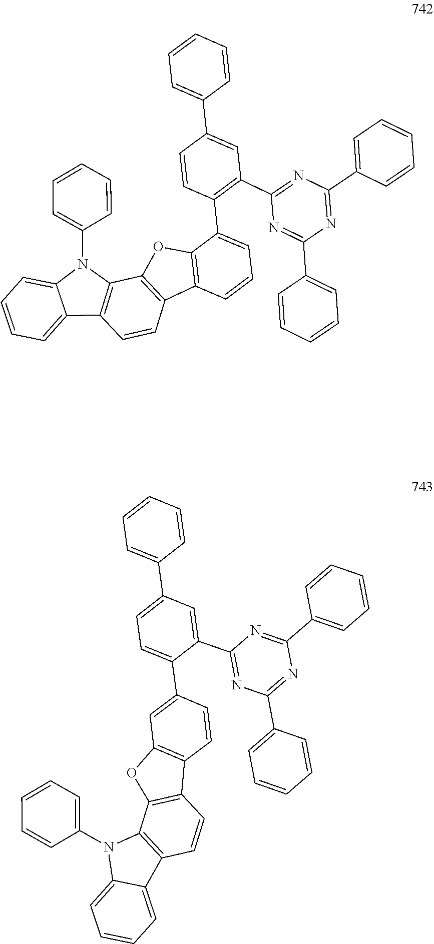 [SMILES: c1ccc(-c2ccc(-c3cccc4c3oc3c4ccc4c5ccccc5n(-c5ccccc5)c43)c(-c3nc(-c4ccccc4)nc(-c4ccccc4)n3)c2)cc1 ; c1ccc(-c2ccc(-c3ccc4c(c3)oc3c4ccc4c5ccccc5n(-c5ccccc5)c43)c(-c3nc(-c4ccccc4)nc(-c4ccccc4)n3)c2)cc1]

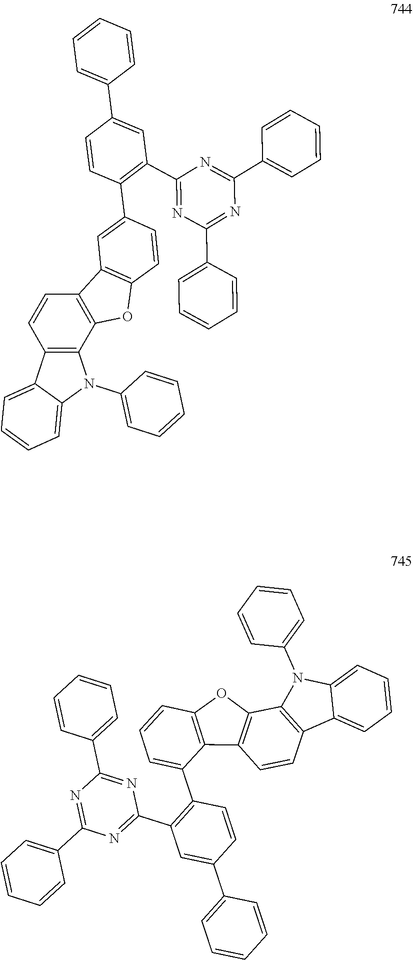 [SMILES: c1ccc(-c2ccc(-c3ccc4oc5c(ccc6c7ccccc7n(-c7ccccc7)c65)c4c3)c(-c3nc(-c4ccccc4)nc(-c4ccccc4)n3)c2)cc1 ; c1ccc(-c2ccc(-c3cccc4oc5c(ccc6c7ccccc7n(-c7ccccc7)c65)c34)c(-c3nc(-c4ccccc4)nc(-c4ccccc4)n3)c2)cc1]

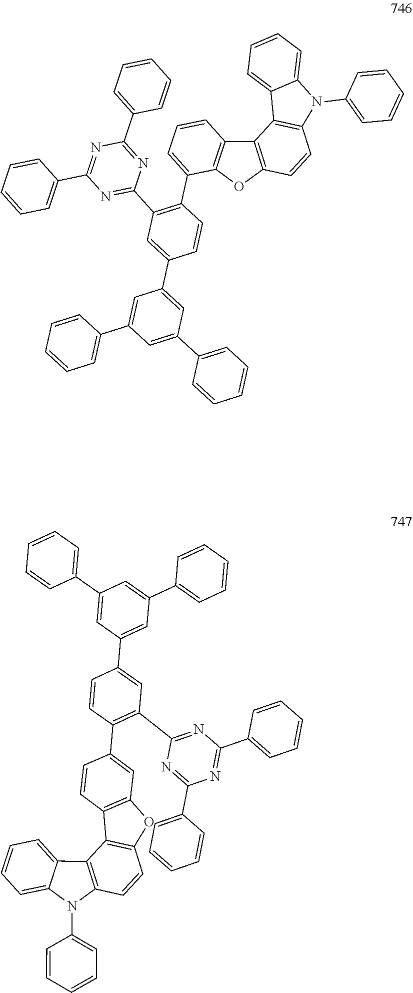 [SMILES: c1ccc(-c2cc(-c3ccccc3)cc(-c3ccc(-c4cccc5c4oc4ccc6c(c7ccccc7n6-c6ccccc6)c45)c(-c4nc(-c5ccccc5)nc(-c5ccccc5)n4)c3)c2)cc1 ; c1ccc(-c2cc(-c3ccccc3)cc(-c3ccc(-c4ccc5c(c4)oc4ccc6c(c7ccccc7n6-c6ccccc6)c45)c(-c4nc(-c5ccccc5)nc(-c5ccccc5)n4)c3)c2)cc1]

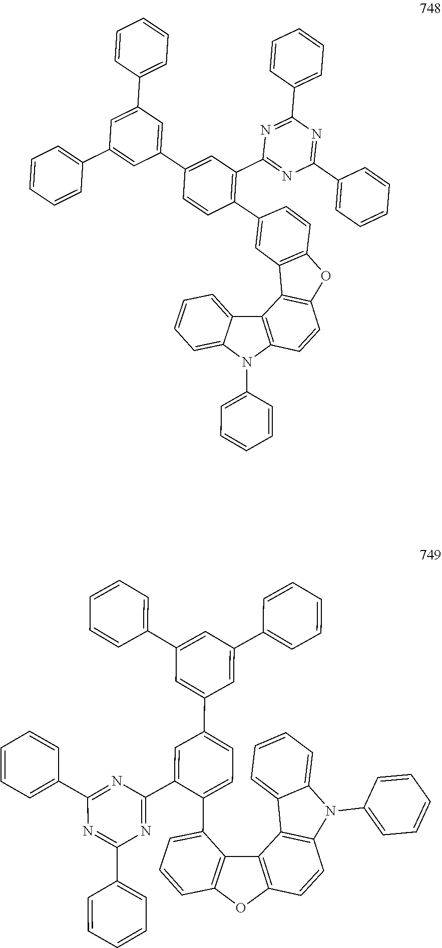

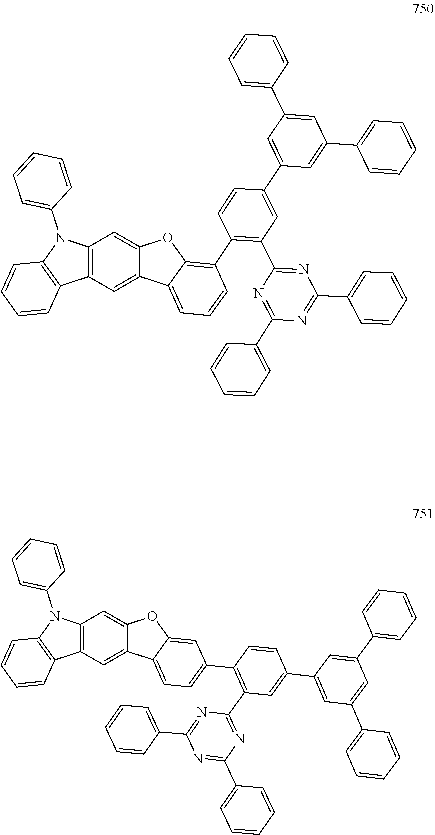 [SMILES: c1ccc(-c2cc(-c3ccccc3)cc(-c3ccc(-c4cccc5c4oc4cc6c(cc45)c4ccccc4n6-c4ccccc4)c(-c4nc(-c5ccccc5)nc(-c5ccccc5)n4)c3)c2)cc1 ; c1ccc(-c2cc(-c3ccccc3)cc(-c3ccc(-c4ccc5c(c4)oc4cc6c(cc45)c4ccccc4n6-c4ccccc4)c(-c4nc(-c5ccccc5)nc(-c5ccccc5)n4)c3)c2)cc1]

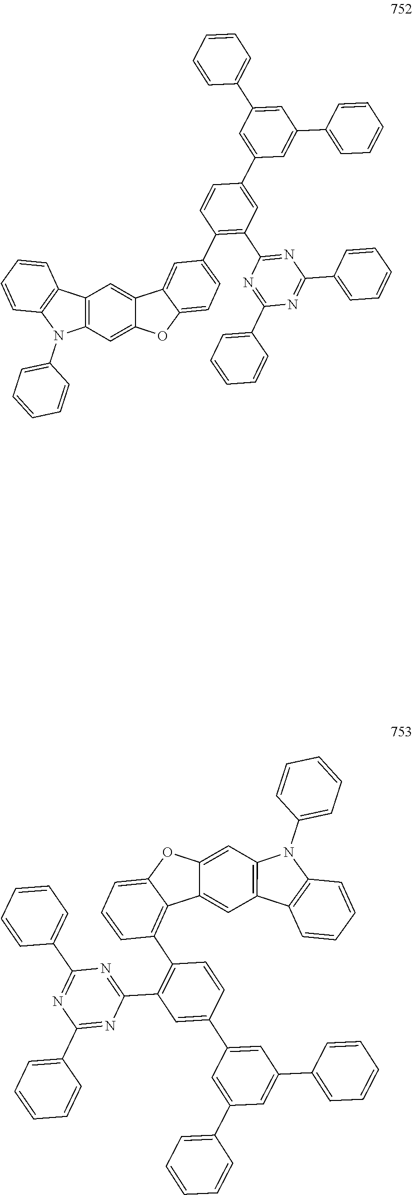 [SMILES: c1ccc(-c2cc(-c3ccccc3)cc(-c3ccc(-c4ccc5oc6cc7c(cc6c5c4)c4ccccc4n7-c4ccccc4)c(-c4nc(-c5ccccc5)nc(-c5ccccc5)n4)c3)c2)cc1 ; c1ccc(-c2cc(-c3ccccc3)cc(-c3ccc(-c4cccc5oc6cc7c(cc6c45)c4ccccc4n7-c4ccccc4)c(-c4nc(-c5ccccc5)nc(-c5ccccc5)n4)c3)c2)cc1]

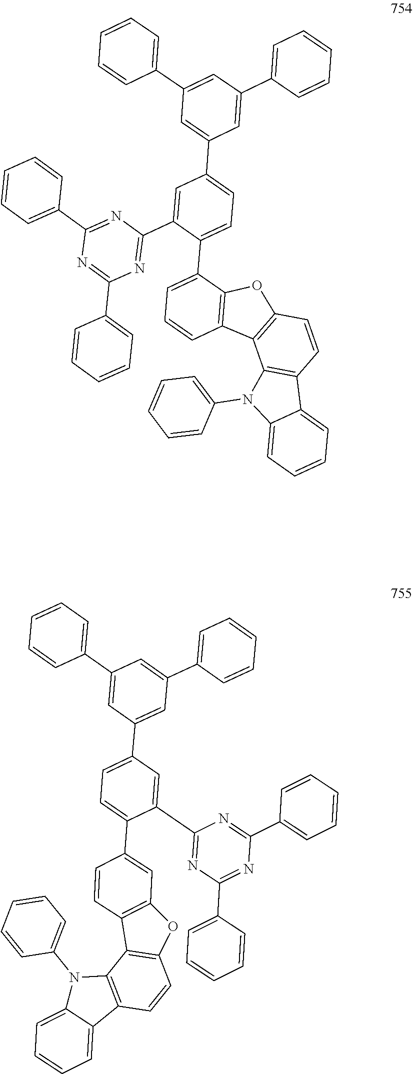 [SMILES: c1ccc(-c2cc(-c3ccccc3)cc(-c3ccc(-c4cccc5c4oc4ccc6c7ccccc7n(-c7ccccc7)c6c45)c(-c4nc(-c5ccccc5)nc(-c5ccccc5)n4)c3)c2)cc1 ; c1ccc(-c2cc(-c3ccccc3)cc(-c3ccc(-c4ccc5c(c4)oc4ccc6c7ccccc7n(-c7ccccc7)c6c45)c(-c4nc(-c5ccccc5)nc(-c5ccccc5)n4)c3)c2)cc1]

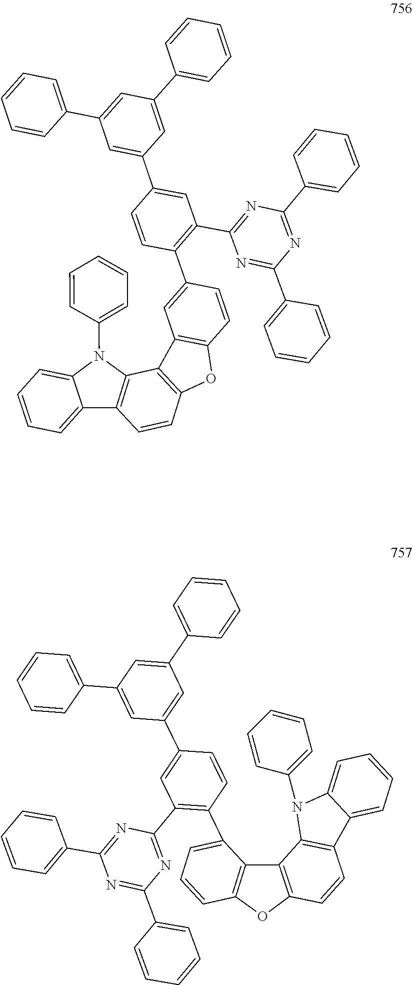 [SMILES: c1ccc(-c2cc(-c3ccccc3)cc(-c3ccc(-c4ccc5oc6ccc7c8ccccc8n(-c8ccccc8)c7c6c5c4)c(-c4nc(-c5ccccc5)nc(-c5ccccc5)n4)c3)c2)cc1 ; c1ccc(-c2cc(-c3ccccc3)cc(-c3ccc(-c4cccc5oc6ccc7c8ccccc8n(-c8ccccc8)c7c6c45)c(-c4nc(-c5ccccc5)nc(-c5ccccc5)n4)c3)c2)cc1]

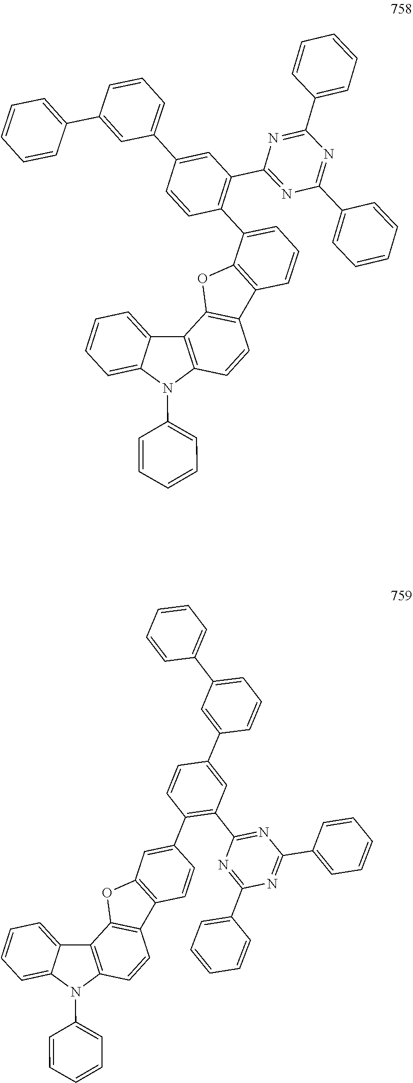 [SMILES: c1ccc(-c2cccc(-c3ccc(-c4cccc5c4oc4c5ccc5c4c4ccccc4n5-c4ccccc4)c(-c4nc(-c5ccccc5)nc(-c5ccccc5)n4)c3)c2)cc1 ; c1ccc(-c2cccc(-c3ccc(-c4ccc5c(c4)oc4c5ccc5c4c4ccccc4n5-c4ccccc4)c(-c4nc(-c5ccccc5)nc(-c5ccccc5)n4)c3)c2)cc1]

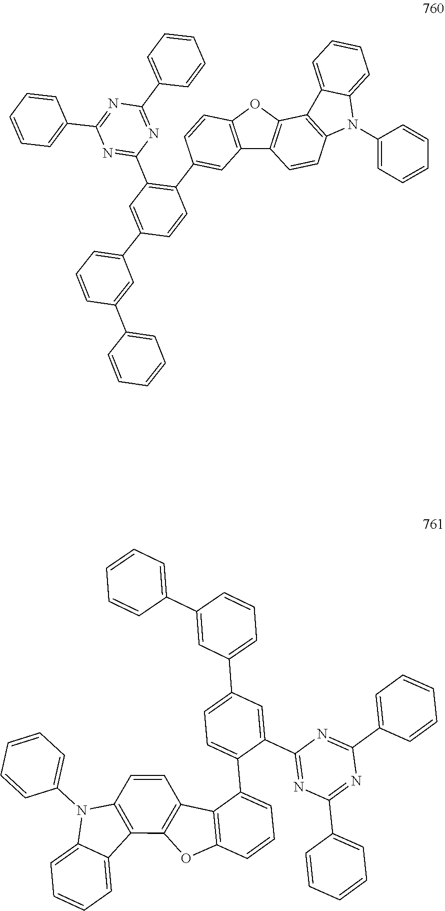 [SMILES: c1ccc(-c2cccc(-c3ccc(-c4ccc5oc6c(ccc7c6c6ccccc6n7-c6ccccc6)c5c4)c(-c4nc(-c5ccccc5)nc(-c5ccccc5)n4)c3)c2)cc1 ; c1ccc(-c2cccc(-c3ccc(-c4cccc5oc6c(ccc7c6c6ccccc6n7-c6ccccc6)c45)c(-c4nc(-c5ccccc5)nc(-c5ccccc5)n4)c3)c2)cc1]

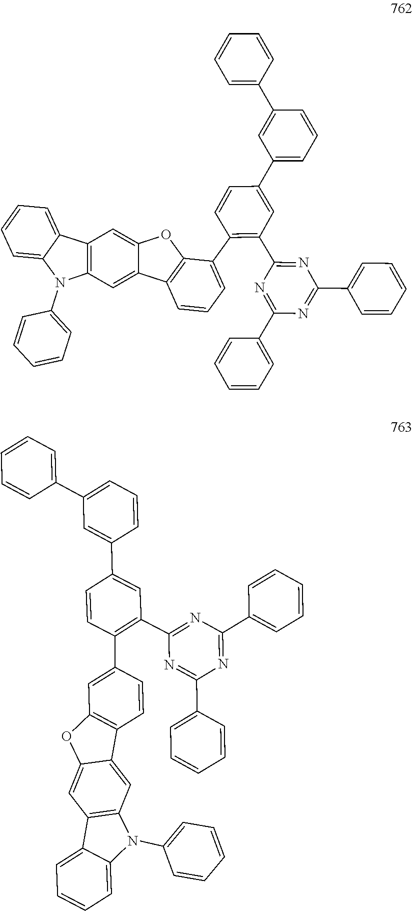 [SMILES: c1ccc(-c2cccc(-c3ccc(-c4cccc5c4oc4cc6c7ccccc7n(-c7ccccc7)c6cc45)c(-c4nc(-c5ccccc5)nc(-c5ccccc5)n4)c3)c2)cc1 ; c1ccc(-c2cccc(-c3ccc(-c4ccc5c(c4)oc4cc6c7ccccc7n(-c7ccccc7)c6cc45)c(-c4nc(-c5ccccc5)nc(-c5ccccc5)n4)c3)c2)cc1]

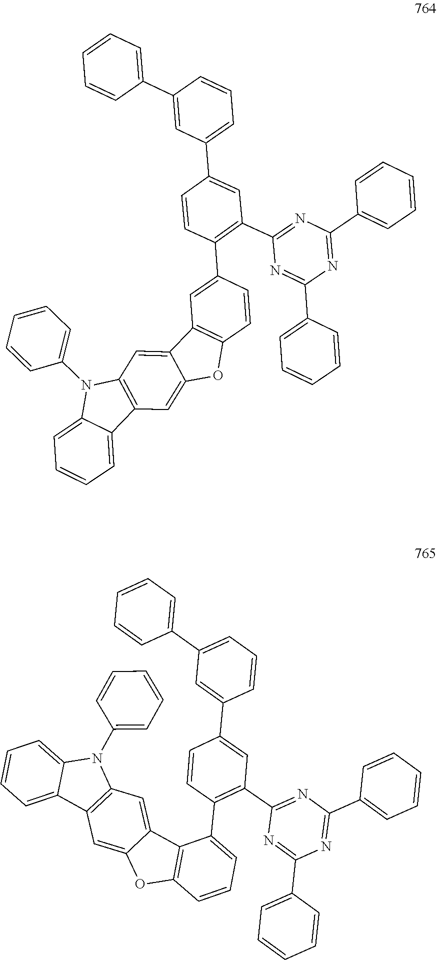 [SMILES: c1ccc(-c2cccc(-c3ccc(-c4ccc5oc6cc7c8ccccc8n(-c8ccccc8)c7cc6c5c4)c(-c4nc(-c5ccccc5)nc(-c5ccccc5)n4)c3)c2)cc1 ; c1ccc(-c2cccc(-c3ccc(-c4cccc5oc6cc7c8ccccc8n(-c8ccccc8)c7cc6c45)c(-c4nc(-c5ccccc5)nc(-c5ccccc5)n4)c3)c2)cc1]

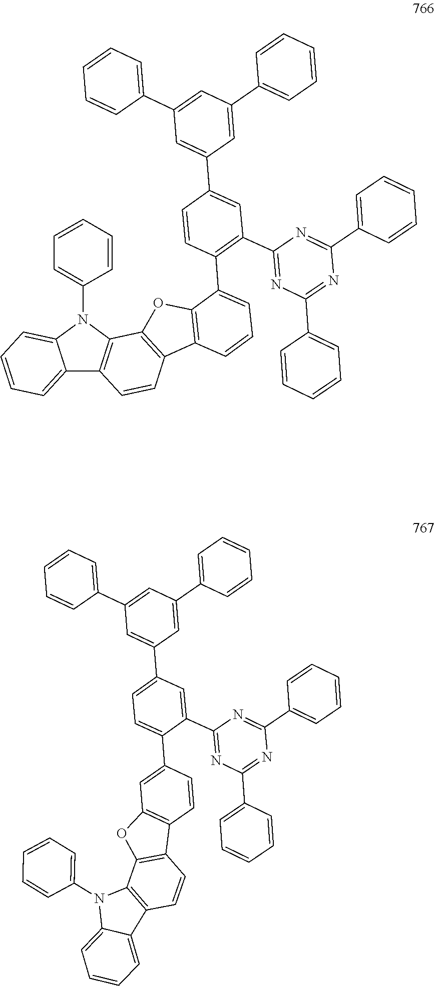 [SMILES: c1ccc(-c2cc(-c3ccccc3)cc(-c3ccc(-c4cccc5c4oc4c5ccc5c6ccccc6n(-c6ccccc6)c54)c(-c4nc(-c5ccccc5)nc(-c5ccccc5)n4)c3)c2)cc1 ; c1ccc(-c2cc(-c3ccccc3)cc(-c3ccc(-c4ccc5c(c4)oc4c5ccc5c6ccccc6n(-c6ccccc6)c54)c(-c4nc(-c5ccccc5)nc(-c5ccccc5)n4)c3)c2)cc1]

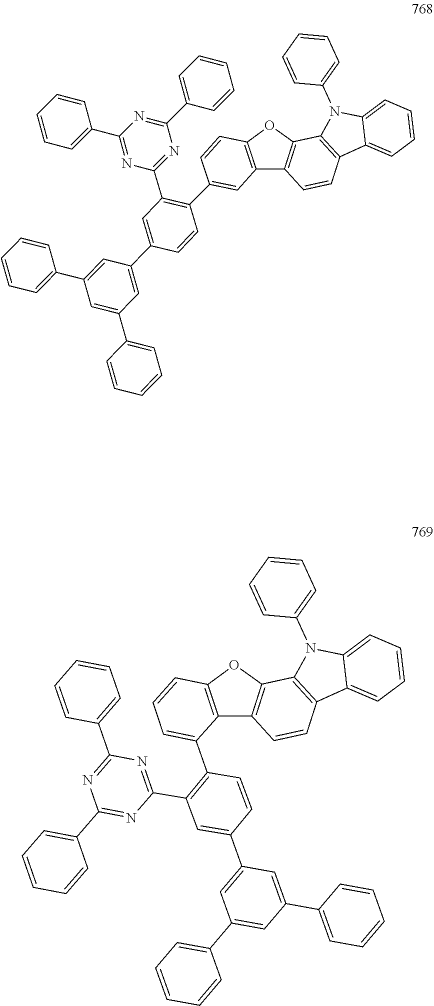 [SMILES: c1ccc(-c2cc(-c3ccccc3)cc(-c3ccc(-c4ccc5oc6c(ccc7c8ccccc8n(-c8ccccc8)c76)c5c4)c(-c4nc(-c5ccccc5)nc(-c5ccccc5)n4)c3)c2)cc1 ; c1ccc(-c2cc(-c3ccccc3)cc(-c3ccc(-c4cccc5oc6c(ccc7c8ccccc8n(-c8ccccc8)c76)c45)c(-c4nc(-c5ccccc5)nc(-c5ccccc5)n4)c3)c2)cc1]

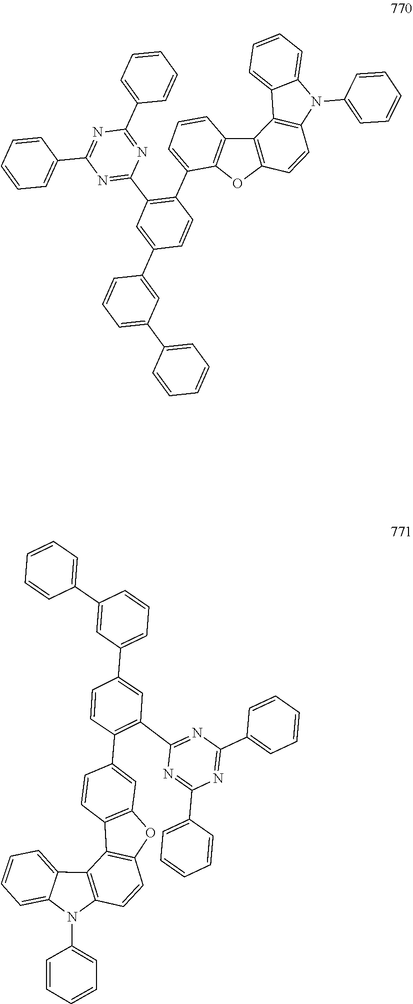 [SMILES: c1ccc(-c2cccc(-c3ccc(-c4cccc5c4oc4ccc6c(c7ccccc7n6-c6ccccc6)c45)c(-c4nc(-c5ccccc5)nc(-c5ccccc5)n4)c3)c2)cc1 ; c1ccc(-c2cccc(-c3ccc(-c4ccc5c(c4)oc4ccc6c(c7ccccc7n6-c6ccccc6)c45)c(-c4nc(-c5ccccc5)nc(-c5ccccc5)n4)c3)c2)cc1]

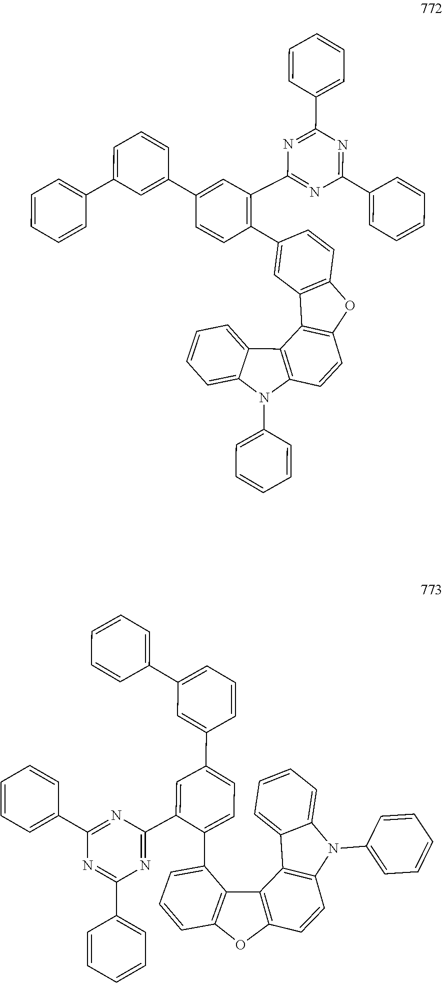 [SMILES: c1ccc(-c2cccc(-c3ccc(-c4ccc5oc6ccc7c(c8ccccc8n7-c7ccccc7)c6c5c4)c(-c4nc(-c5ccccc5)nc(-c5ccccc5)n4)c3)c2)cc1 ; c1ccc(-c2cccc(-c3ccc(-c4cccc5oc6ccc7c(c8ccccc8n7-c7ccccc7)c6c45)c(-c4nc(-c5ccccc5)nc(-c5ccccc5)n4)c3)c2)cc1]

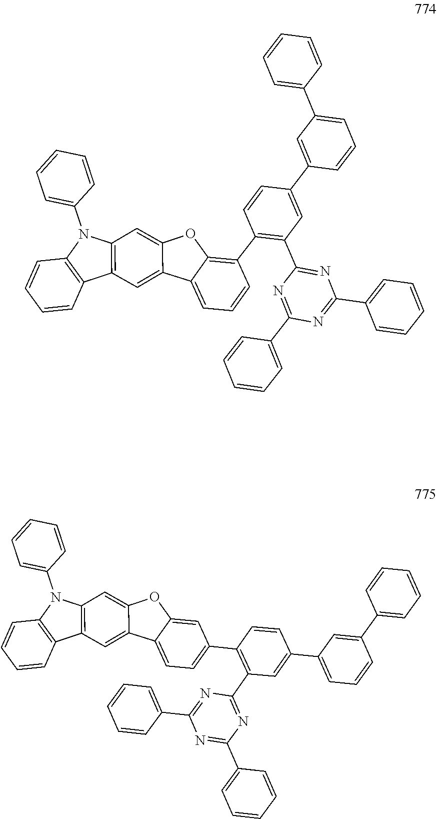 [SMILES: c1ccc(-c2cccc(-c3ccc(-c4cccc5c4oc4cc6c(cc45)c4ccccc4n6-c4ccccc4)c(-c4nc(-c5ccccc5)nc(-c5ccccc5)n4)c3)c2)cc1 ; c1ccc(-c2cccc(-c3ccc(-c4ccc5c(c4)oc4cc6c(cc45)c4ccccc4n6-c4ccccc4)c(-c4nc(-c5ccccc5)nc(-c5ccccc5)n4)c3)c2)cc1]

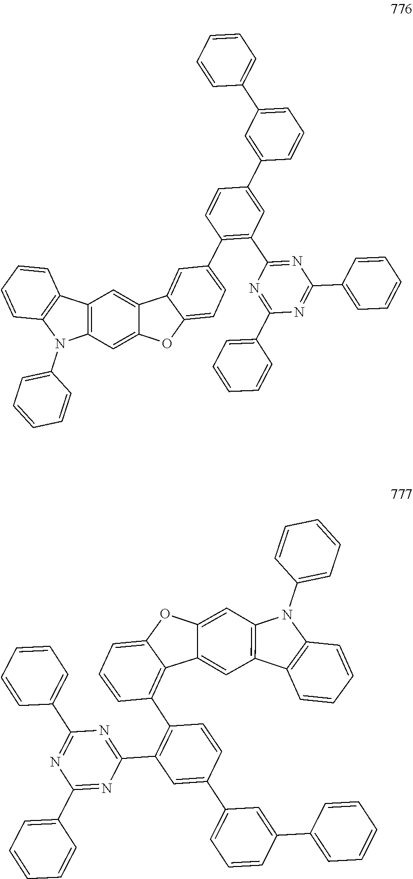 [SMILES: c1ccc(-c2cccc(-c3ccc(-c4ccc5oc6cc7c(cc6c5c4)c4ccccc4n7-c4ccccc4)c(-c4nc(-c5ccccc5)nc(-c5ccccc5)n4)c3)c2)cc1 ; c1ccc(-c2cccc(-c3ccc(-c4cccc5oc6cc7c(cc6c45)c4ccccc4n7-c4ccccc4)c(-c4nc(-c5ccccc5)nc(-c5ccccc5)n4)c3)c2)cc1]

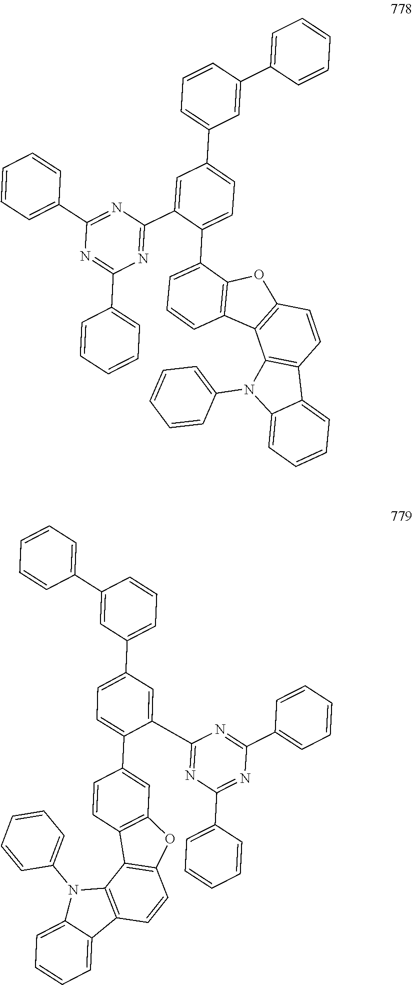 [SMILES: c1ccc(-c2cccc(-c3ccc(-c4cccc5c4oc4ccc6c7ccccc7n(-c7ccccc7)c6c45)c(-c4nc(-c5ccccc5)nc(-c5ccccc5)n4)c3)c2)cc1 ; c1ccc(-c2cccc(-c3ccc(-c4ccc5c(c4)oc4ccc6c7ccccc7n(-c7ccccc7)c6c45)c(-c4nc(-c5ccccc5)nc(-c5ccccc5)n4)c3)c2)cc1]

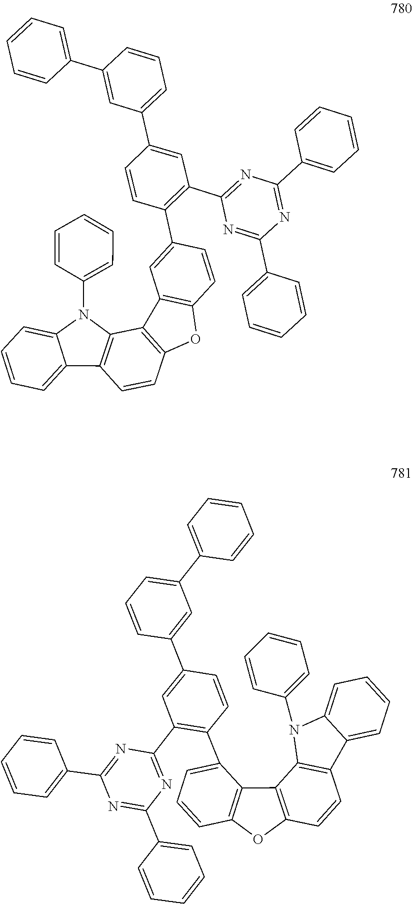 [SMILES: c1ccc(-c2cccc(-c3ccc(-c4ccc5oc6ccc7c8ccccc8n(-c8ccccc8)c7c6c5c4)c(-c4nc(-c5ccccc5)nc(-c5ccccc5)n4)c3)c2)cc1 ; c1ccc(-c2cccc(-c3ccc(-c4cccc5oc6ccc7c8ccccc8n(-c8ccccc8)c7c6c45)c(-c4nc(-c5ccccc5)nc(-c5ccccc5)n4)c3)c2)cc1]

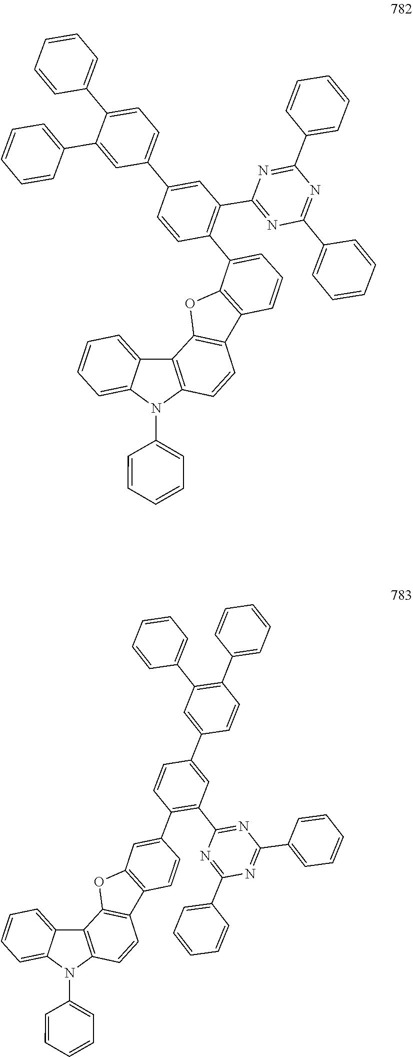 [SMILES: c1ccc(-c2nc(-c3ccccc3)nc(-c3cc(-c4ccc(-c5ccccc5)c(-c5ccccc5)c4)ccc3-c3cccc4c3oc3c4ccc4c3c3ccccc3n4-c3ccccc3)n2)cc1 ; c1ccc(-c2nc(-c3ccccc3)nc(-c3cc(-c4ccc(-c5ccccc5)c(-c5ccccc5)c4)ccc3-c3ccc4c(c3)oc3c4ccc4c3c3ccccc3n4-c3ccccc3)n2)cc1]

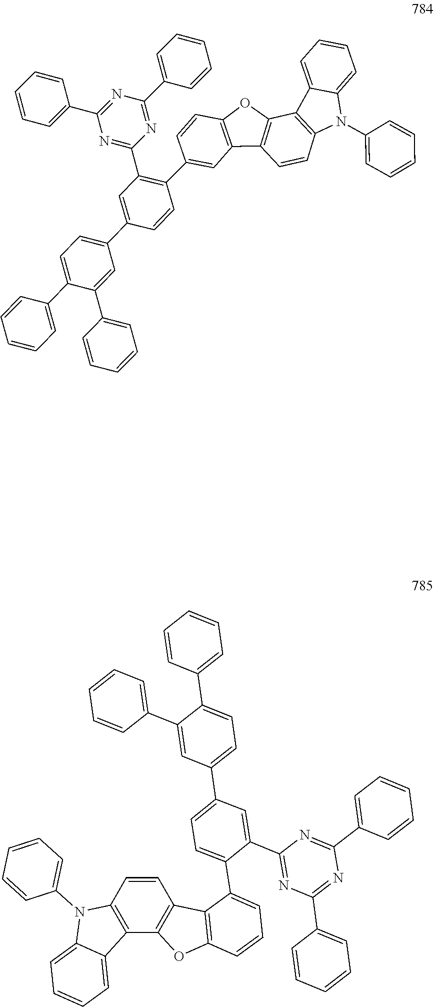 [SMILES: c1ccc(-c2nc(-c3ccccc3)nc(-c3cc(-c4ccc(-c5ccccc5)c(-c5ccccc5)c4)ccc3-c3ccc4oc5c(ccc6c5c5ccccc5n6-c5ccccc5)c4c3)n2)cc1 ; c1ccc(-c2nc(-c3ccccc3)nc(-c3cc(-c4ccc(-c5ccccc5)c(-c5ccccc5)c4)ccc3-c3cccc4oc5c(ccc6c5c5ccccc5n6-c5ccccc5)c34)n2)cc1]

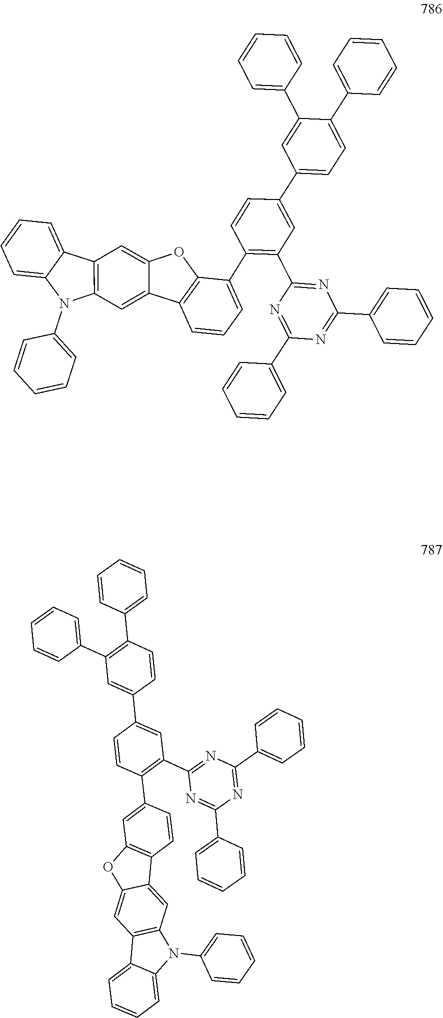 [SMILES: c1ccc(-c2nc(-c3ccccc3)nc(-c3cc(-c4ccc(-c5ccccc5)c(-c5ccccc5)c4)ccc3-c3cccc4c3oc3cc5c6ccccc6n(-c6ccccc6)c5cc34)n2)cc1 ; c1ccc(-c2nc(-c3ccccc3)nc(-c3cc(-c4ccc(-c5ccccc5)c(-c5ccccc5)c4)ccc3-c3ccc4c(c3)oc3cc5c6ccccc6n(-c6ccccc6)c5cc34)n2)cc1]

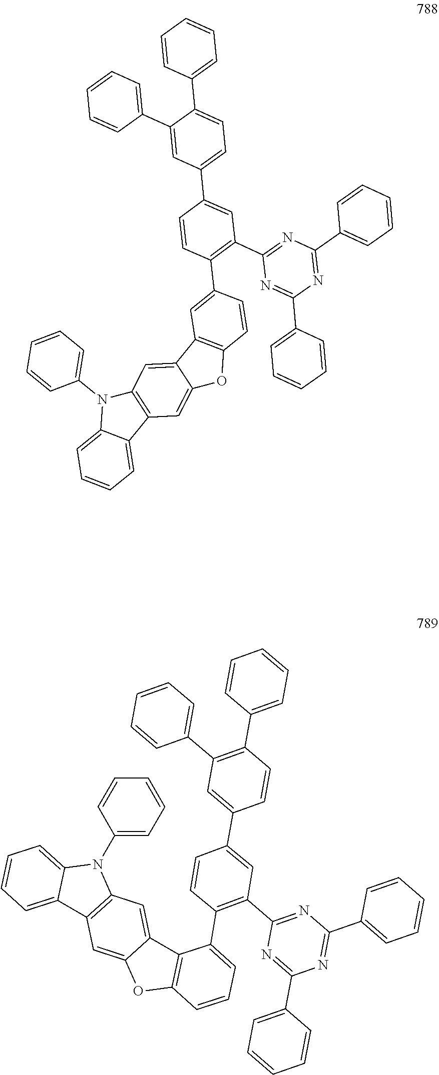 [SMILES: c1ccc(-c2nc(-c3ccccc3)nc(-c3cc(-c4ccc(-c5ccccc5)c(-c5ccccc5)c4)ccc3-c3ccc4oc5cc6c7ccccc7n(-c7ccccc7)c6cc5c4c3)n2)cc1 ; c1ccc(-c2nc(-c3ccccc3)nc(-c3cc(-c4ccc(-c5ccccc5)c(-c5ccccc5)c4)ccc3-c3cccc4oc5cc6c7ccccc7n(-c7ccccc7)c6cc5c34)n2)cc1]

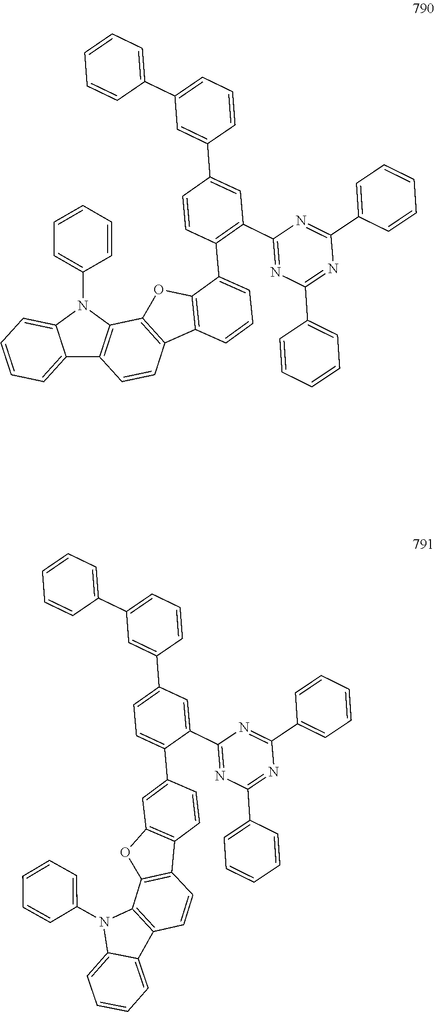 [SMILES: c1ccc(-c2cccc(-c3ccc(-c4cccc5c4oc4c5ccc5c6ccccc6n(-c6ccccc6)c54)c(-c4nc(-c5ccccc5)nc(-c5ccccc5)n4)c3)c2)cc1 ; c1ccc(-c2cccc(-c3ccc(-c4ccc5c(c4)oc4c5ccc5c6ccccc6n(-c6ccccc6)c54)c(-c4nc(-c5ccccc5)nc(-c5ccccc5)n4)c3)c2)cc1]

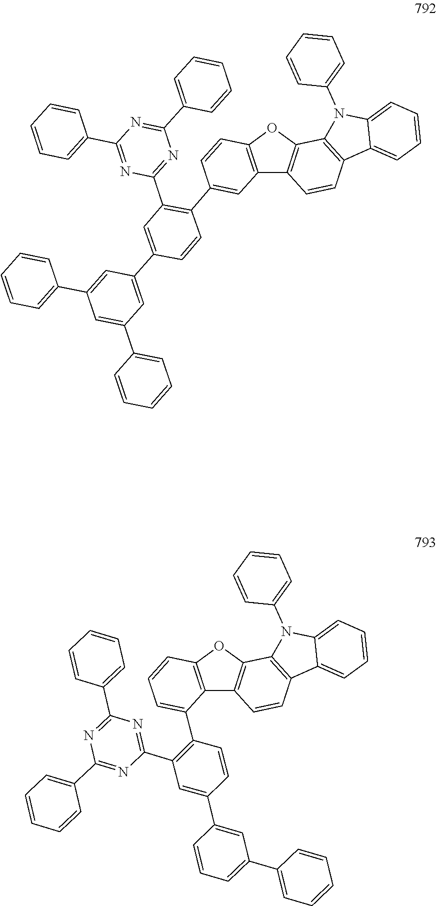 [SMILES: c1ccc(-c2cc(-c3ccccc3)cc(-c3ccc(-c4ccc5oc6c(ccc7c8ccccc8n(-c8ccccc8)c76)c5c4)c(-c4nc(-c5ccccc5)nc(-c5ccccc5)n4)c3)c2)cc1 ; c1ccc(-c2cccc(-c3ccc(-c4cccc5oc6c(ccc7c8ccccc8n(-c8ccccc8)c76)c45)c(-c4nc(-c5ccccc5)nc(-c5ccccc5)n4)c3)c2)cc1]

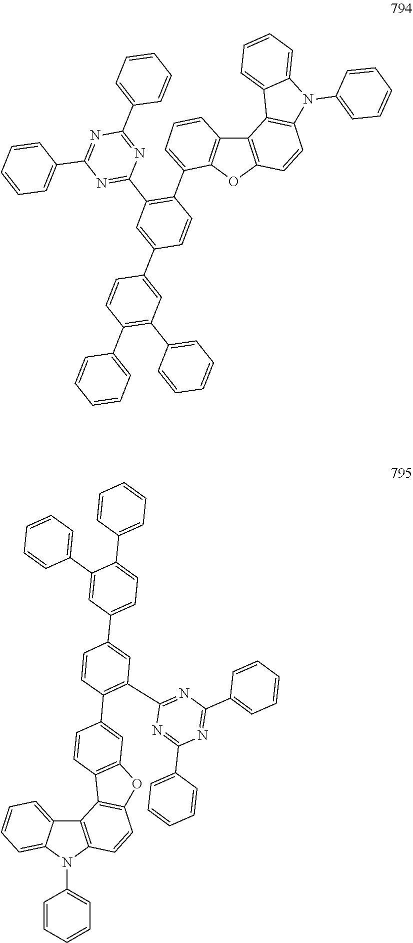 [SMILES: c1ccc(-c2nc(-c3ccccc3)nc(-c3cc(-c4ccc(-c5ccccc5)c(-c5ccccc5)c4)ccc3-c3cccc4c3oc3ccc5c(c6ccccc6n5-c5ccccc5)c34)n2)cc1 ; c1ccc(-c2nc(-c3ccccc3)nc(-c3cc(-c4ccc(-c5ccccc5)c(-c5ccccc5)c4)ccc3-c3ccc4c(c3)oc3ccc5c(c6ccccc6n5-c5ccccc5)c34)n2)cc1]

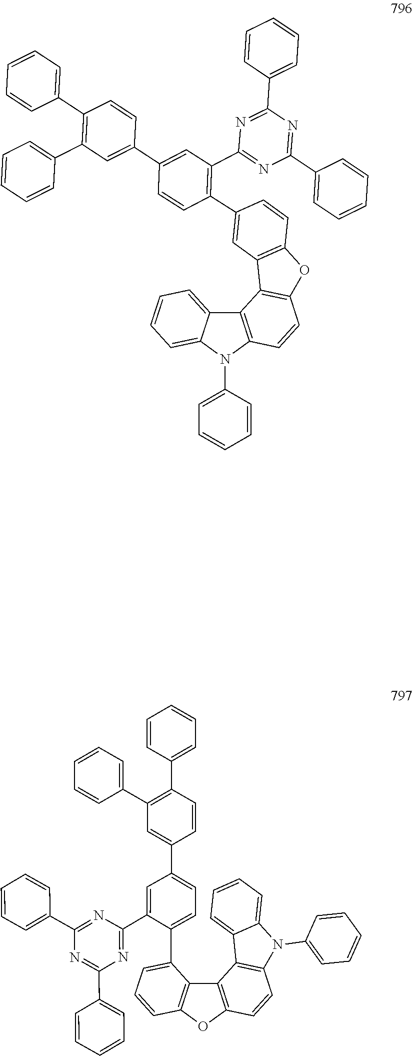 [SMILES: c1ccc(-c2nc(-c3ccccc3)nc(-c3cc(-c4ccc(-c5ccccc5)c(-c5ccccc5)c4)ccc3-c3ccc4oc5ccc6c(c7ccccc7n6-c6ccccc6)c5c4c3)n2)cc1 ; c1ccc(-c2nc(-c3ccccc3)nc(-c3cc(-c4ccc(-c5ccccc5)c(-c5ccccc5)c4)ccc3-c3cccc4oc5ccc6c(c7ccccc7n6-c6ccccc6)c5c34)n2)cc1]

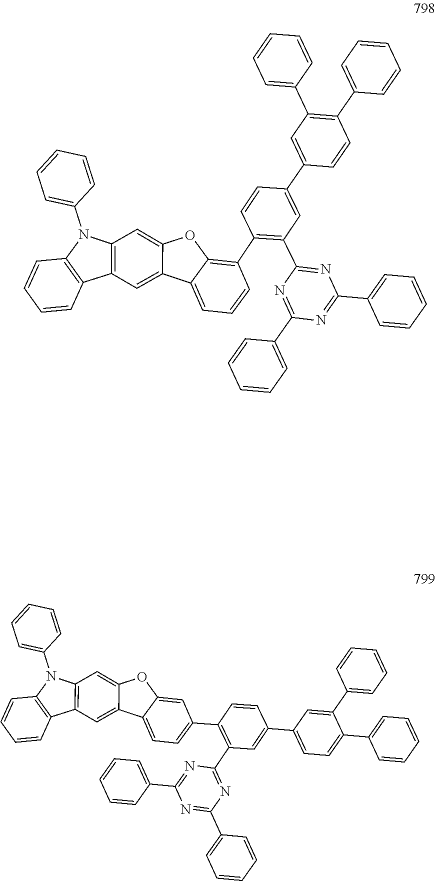 [SMILES: c1ccc(-c2nc(-c3ccccc3)nc(-c3cc(-c4ccc(-c5ccccc5)c(-c5ccccc5)c4)ccc3-c3cccc4c3oc3cc5c(cc34)c3ccccc3n5-c3ccccc3)n2)cc1 ; c1ccc(-c2nc(-c3ccccc3)nc(-c3cc(-c4ccc(-c5ccccc5)c(-c5ccccc5)c4)ccc3-c3ccc4c(c3)oc3cc5c(cc34)c3ccccc3n5-c3ccccc3)n2)cc1]

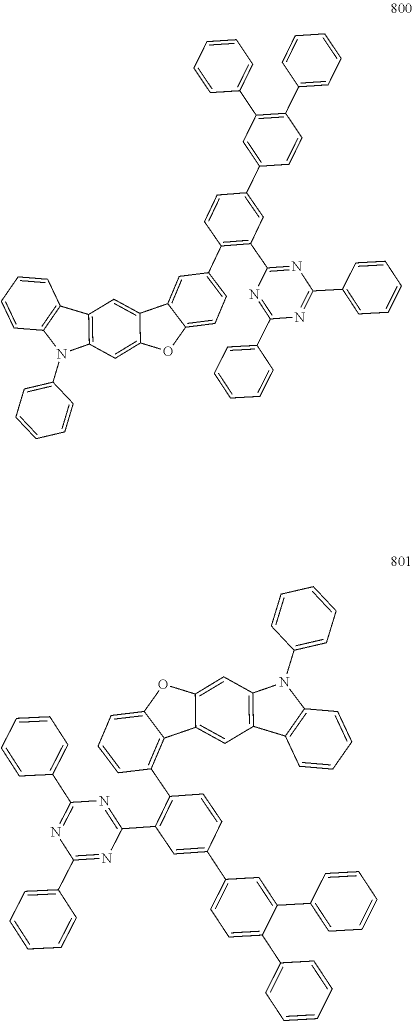 [SMILES: c1ccc(-c2nc(-c3ccccc3)nc(-c3cc(-c4ccc(-c5ccccc5)c(-c5ccccc5)c4)ccc3-c3ccc4oc5cc6c(cc5c4c3)c3ccccc3n6-c3ccccc3)n2)cc1 ; c1ccc(-c2nc(-c3ccccc3)nc(-c3cc(-c4ccc(-c5ccccc5)c(-c5ccccc5)c4)ccc3-c3cccc4oc5cc6c(cc5c34)c3ccccc3n6-c3ccccc3)n2)cc1]

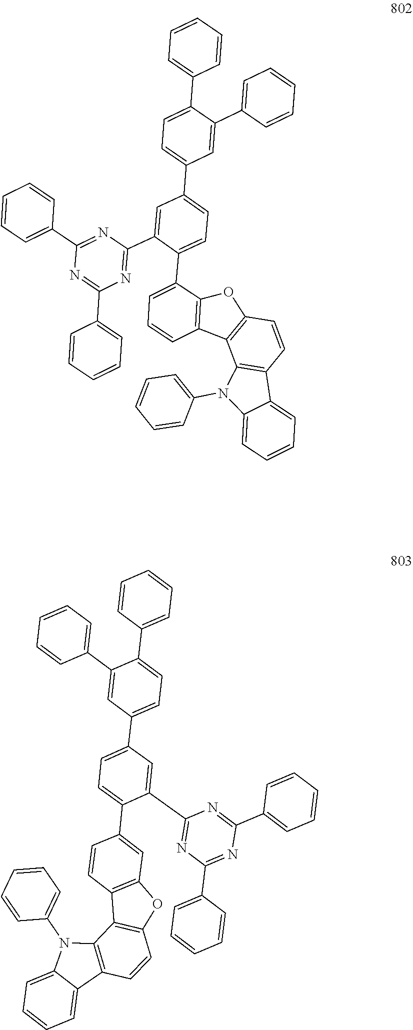 [SMILES: c1ccc(-c2nc(-c3ccccc3)nc(-c3cc(-c4ccc(-c5ccccc5)c(-c5ccccc5)c4)ccc3-c3cccc4c3oc3ccc5c6ccccc6n(-c6ccccc6)c5c34)n2)cc1 ; c1ccc(-c2nc(-c3ccccc3)nc(-c3cc(-c4ccc(-c5ccccc5)c(-c5ccccc5)c4)ccc3-c3ccc4c(c3)oc3ccc5c6ccccc6n(-c6ccccc6)c5c34)n2)cc1]

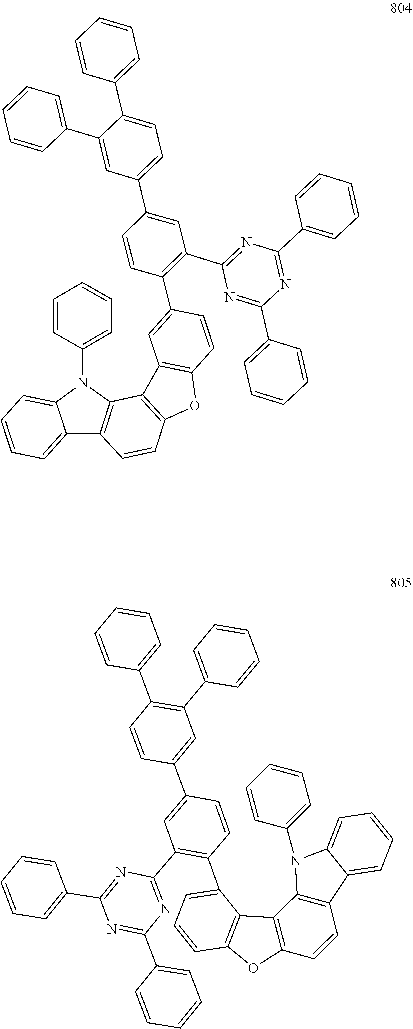 [SMILES: c1ccc(-c2nc(-c3ccccc3)nc(-c3cc(-c4ccc(-c5ccccc5)c(-c5ccccc5)c4)ccc3-c3ccc4oc5ccc6c7ccccc7n(-c7ccccc7)c6c5c4c3)n2)cc1 ; c1ccc(-c2nc(-c3ccccc3)nc(-c3cc(-c4ccc(-c5ccccc5)c(-c5ccccc5)c4)ccc3-c3cccc4oc5ccc6c7ccccc7n(-c7ccccc7)c6c5c34)n2)cc1]

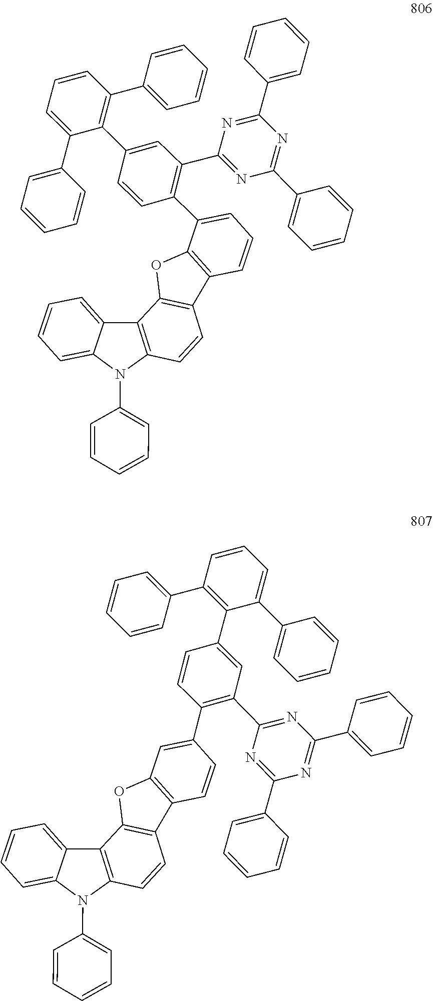 [SMILES: c1ccc(-c2nc(-c3ccccc3)nc(-c3cc(-c4c(-c5ccccc5)cccc4-c4ccccc4)ccc3-c3cccc4c3oc3c4ccc4c3c3ccccc3n4-c3ccccc3)n2)cc1 ; c1ccc(-c2nc(-c3ccccc3)nc(-c3cc(-c4c(-c5ccccc5)cccc4-c4ccccc4)ccc3-c3ccc4c(c3)oc3c4ccc4c3c3ccccc3n4-c3ccccc3)n2)cc1]

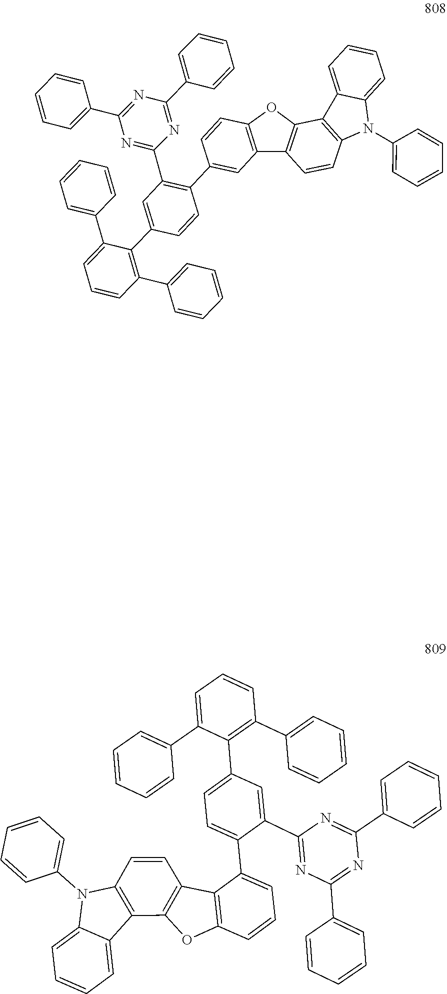 [SMILES: c1ccc(-c2nc(-c3ccccc3)nc(-c3cc(-c4c(-c5ccccc5)cccc4-c4ccccc4)ccc3-c3ccc4oc5c(ccc6c5c5ccccc5n6-c5ccccc5)c4c3)n2)cc1 ; c1ccc(-c2nc(-c3ccccc3)nc(-c3cc(-c4c(-c5ccccc5)cccc4-c4ccccc4)ccc3-c3cccc4oc5c(ccc6c5c5ccccc5n6-c5ccccc5)c34)n2)cc1]

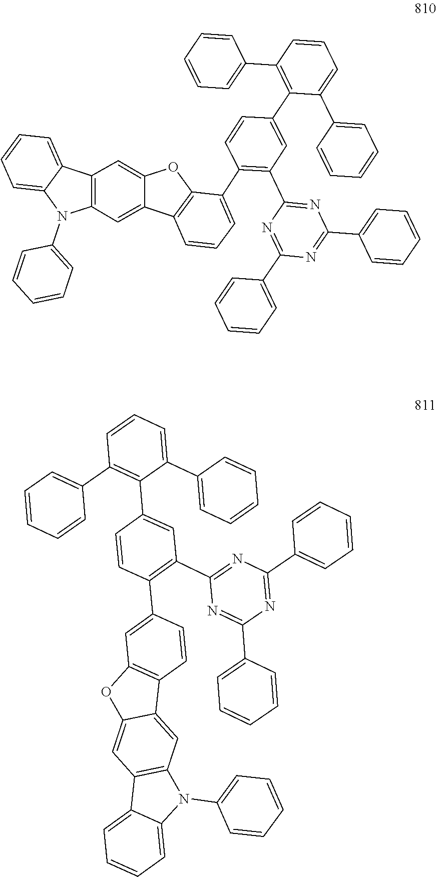 [SMILES: c1ccc(-c2nc(-c3ccccc3)nc(-c3cc(-c4c(-c5ccccc5)cccc4-c4ccccc4)ccc3-c3cccc4c3oc3cc5c6ccccc6n(-c6ccccc6)c5cc34)n2)cc1 ; c1ccc(-c2nc(-c3ccccc3)nc(-c3cc(-c4c(-c5ccccc5)cccc4-c4ccccc4)ccc3-c3ccc4c(c3)oc3cc5c6ccccc6n(-c6ccccc6)c5cc34)n2)cc1]

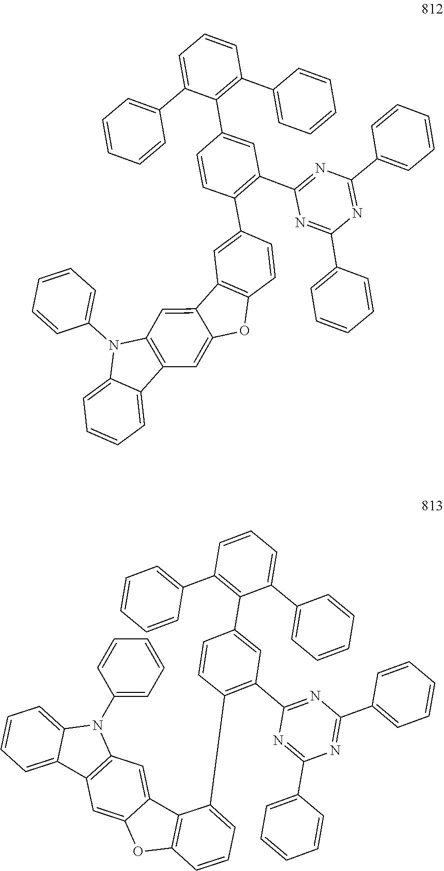 [SMILES: c1ccc(-c2nc(-c3ccccc3)nc(-c3cc(-c4c(-c5ccccc5)cccc4-c4ccccc4)ccc3-c3ccc4oc5cc6c7ccccc7n(-c7ccccc7)c6cc5c4c3)n2)cc1 ; c1ccc(-c2nc(-c3ccccc3)nc(-c3cc(-c4c(-c5ccccc5)cccc4-c4ccccc4)ccc3-c3cccc4oc5cc6c7ccccc7n(-c7ccccc7)c6cc5c34)n2)cc1]

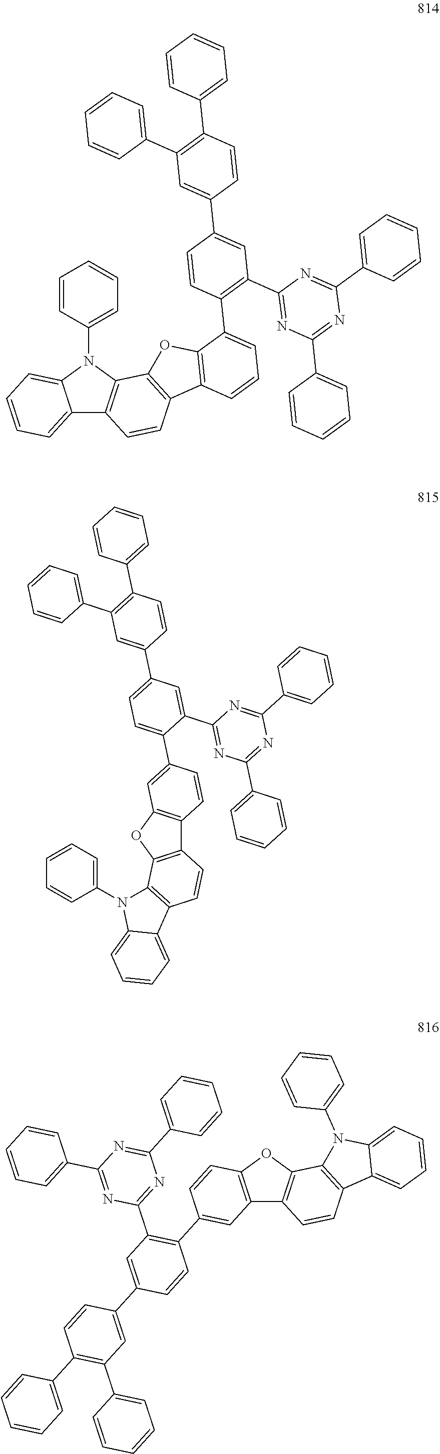 [SMILES: c1ccc(-c2nc(-c3ccccc3)nc(-c3cc(-c4ccc(-c5ccccc5)c(-c5ccccc5)c4)ccc3-c3cccc4c3oc3c4ccc4c5ccccc5n(-c5ccccc5)c43)n2)cc1 ; c1ccc(-c2nc(-c3ccccc3)nc(-c3cc(-c4ccc(-c5ccccc5)c(-c5ccccc5)c4)ccc3-c3ccc4c(c3)oc3c4ccc4c5ccccc5n(-c5ccccc5)c43)n2)cc1 ; c1ccc(-c2nc(-c3ccccc3)nc(-c3cc(-c4ccc(-c5ccccc5)c(-c5ccccc5)c4)ccc3-c3ccc4oc5c(ccc6c7ccccc7n(-c7ccccc7)c65)c4c3)n2)cc1]

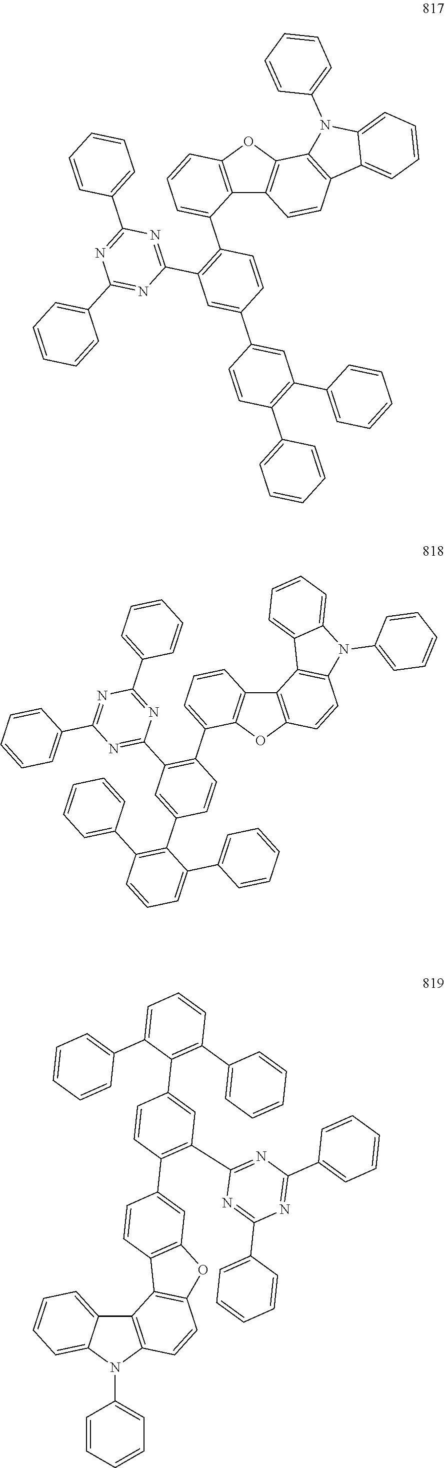 [SMILES: c1ccc(-c2nc(-c3ccccc3)nc(-c3cc(-c4ccc(-c5ccccc5)c(-c5ccccc5)c4)ccc3-c3cccc4oc5c(ccc6c7ccccc7n(-c7ccccc7)c65)c34)n2)cc1 ; c1ccc(-c2nc(-c3ccccc3)nc(-c3cc(-c4c(-c5ccccc5)cccc4-c4ccccc4)ccc3-c3cccc4c3oc3ccc5c(c6ccccc6n5-c5ccccc5)c34)n2)cc1 ; c1ccc(-c2nc(-c3ccccc3)nc(-c3cc(-c4c(-c5ccccc5)cccc4-c4ccccc4)ccc3-c3ccc4c(c3)oc3ccc5c(c6ccccc6n5-c5ccccc5)c34)n2)cc1]

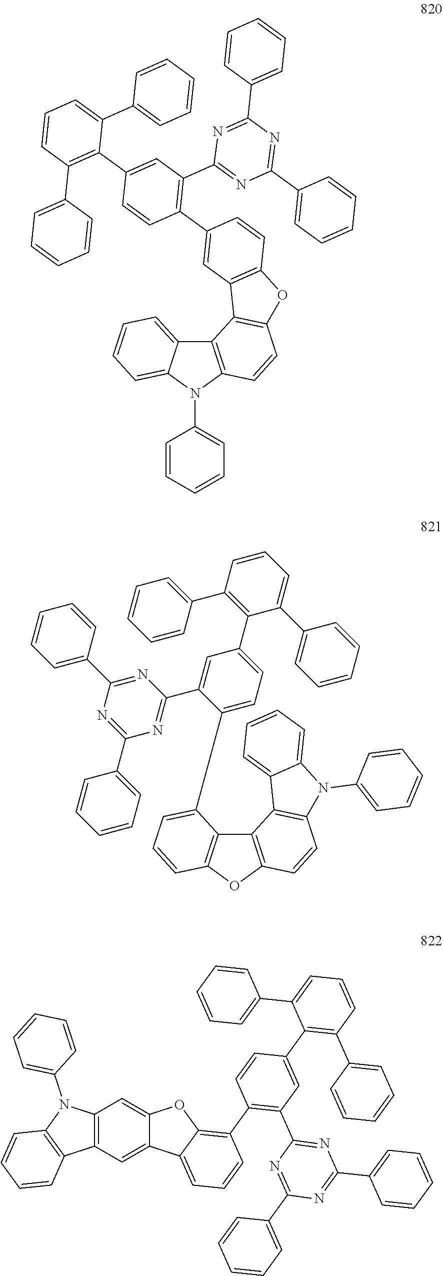 [SMILES: c1ccc(-c2nc(-c3ccccc3)nc(-c3cc(-c4c(-c5ccccc5)cccc4-c4ccccc4)ccc3-c3ccc4oc5ccc6c(c7ccccc7n6-c6ccccc6)c5c4c3)n2)cc1 ; c1ccc(-c2nc(-c3ccccc3)nc(-c3cc(-c4c(-c5ccccc5)cccc4-c4ccccc4)ccc3-c3cccc4oc5ccc6c(c7ccccc7n6-c6ccccc6)c5c34)n2)cc1 ; c1ccc(-c2nc(-c3ccccc3)nc(-c3cc(-c4c(-c5ccccc5)cccc4-c4ccccc4)ccc3-c3cccc4c3oc3cc5c(cc34)c3ccccc3n5-c3ccccc3)n2)cc1]

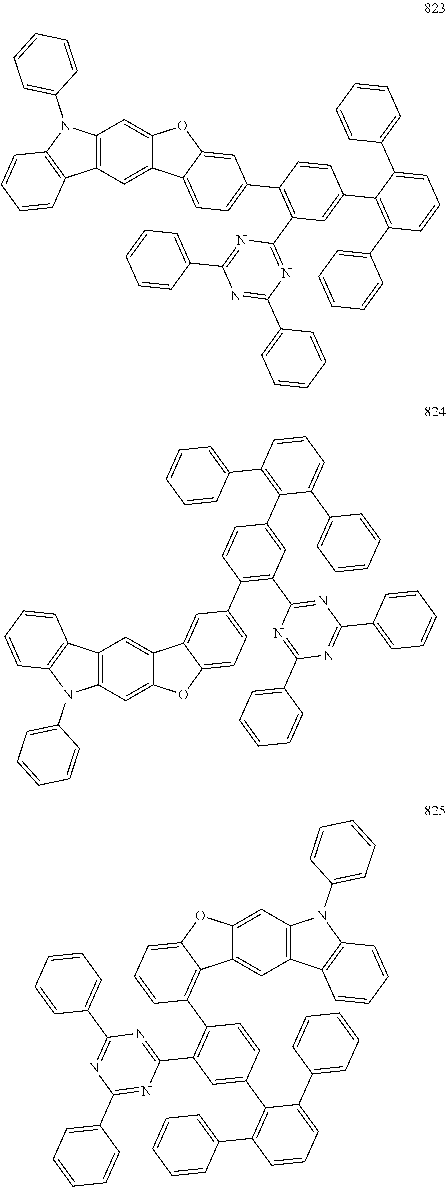 [SMILES: c1ccc(-c2nc(-c3ccccc3)nc(-c3cc(-c4c(-c5ccccc5)cccc4-c4ccccc4)ccc3-c3ccc4c(c3)oc3cc5c(cc34)c3ccccc3n5-c3ccccc3)n2)cc1 ; c1ccc(-c2nc(-c3ccccc3)nc(-c3cc(-c4c(-c5ccccc5)cccc4-c4ccccc4)ccc3-c3ccc4oc5cc6c(cc5c4c3)c3ccccc3n6-c3ccccc3)n2)cc1 ; c1ccc(-c2nc(-c3ccccc3)nc(-c3cc(-c4c(-c5ccccc5)cccc4-c4ccccc4)ccc3-c3cccc4oc5cc6c(cc5c34)c3ccccc3n6-c3ccccc3)n2)cc1]

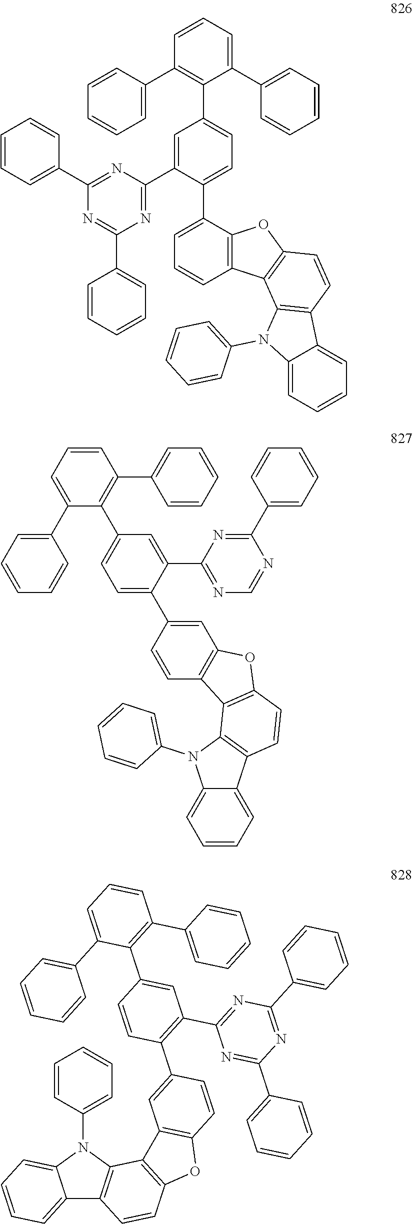

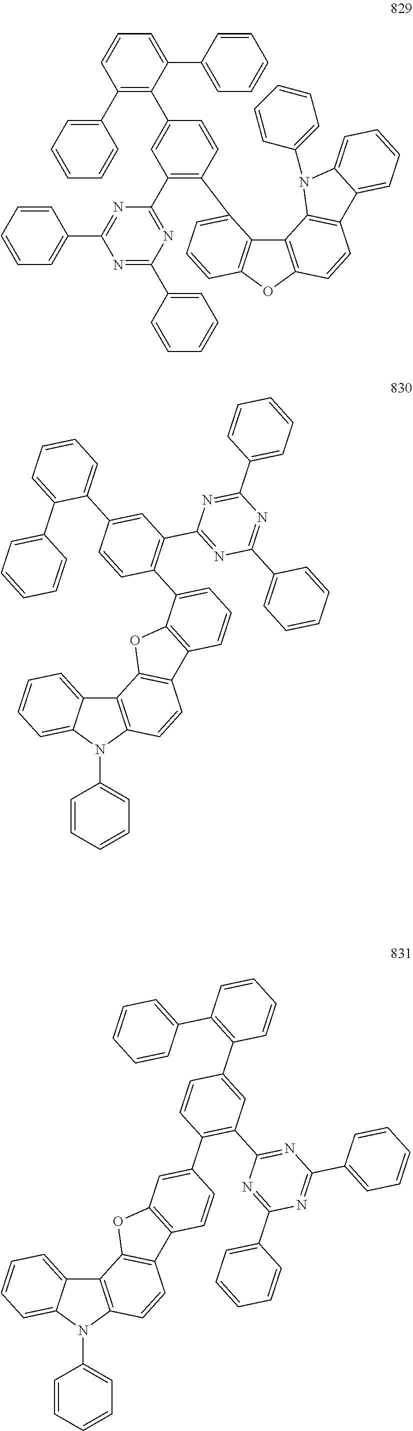 [SMILES: c1ccc(-c2nc(-c3ccccc3)nc(-c3cc(-c4c(-c5ccccc5)cccc4-c4ccccc4)ccc3-c3cccc4oc5ccc6c7ccccc7n(-c7ccccc7)c6c5c34)n2)cc1 ; c1ccc(-c2nc(-c3ccccc3)nc(-c3cc(-c4ccccc4-c4ccccc4)ccc3-c3cccc4c3oc3c4ccc4c3c3ccccc3n4-c3ccccc3)n2)cc1 ; c1ccc(-c2nc(-c3ccccc3)nc(-c3cc(-c4ccccc4-c4ccccc4)ccc3-c3ccc4c(c3)oc3c4ccc4c3c3ccccc3n4-c3ccccc3)n2)cc1]

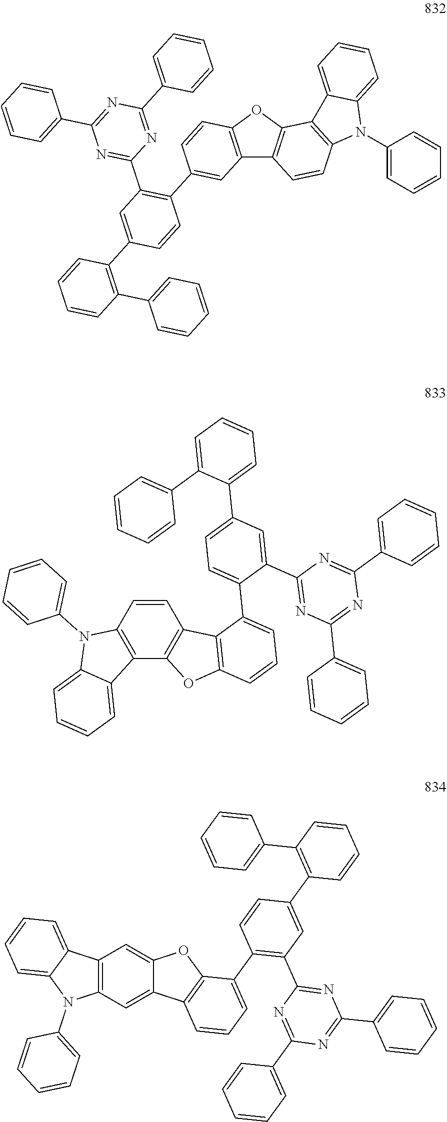 [SMILES: c1ccc(-c2nc(-c3ccccc3)nc(-c3cc(-c4ccccc4-c4ccccc4)ccc3-c3ccc4oc5c(ccc6c5c5ccccc5n6-c5ccccc5)c4c3)n2)cc1 ; c1ccc(-c2nc(-c3ccccc3)nc(-c3cc(-c4ccccc4-c4ccccc4)ccc3-c3cccc4oc5c(ccc6c5c5ccccc5n6-c5ccccc5)c34)n2)cc1 ; c1ccc(-c2nc(-c3ccccc3)nc(-c3cc(-c4ccccc4-c4ccccc4)ccc3-c3cccc4c3oc3cc5c6ccccc6n(-c6ccccc6)c5cc34)n2)cc1]

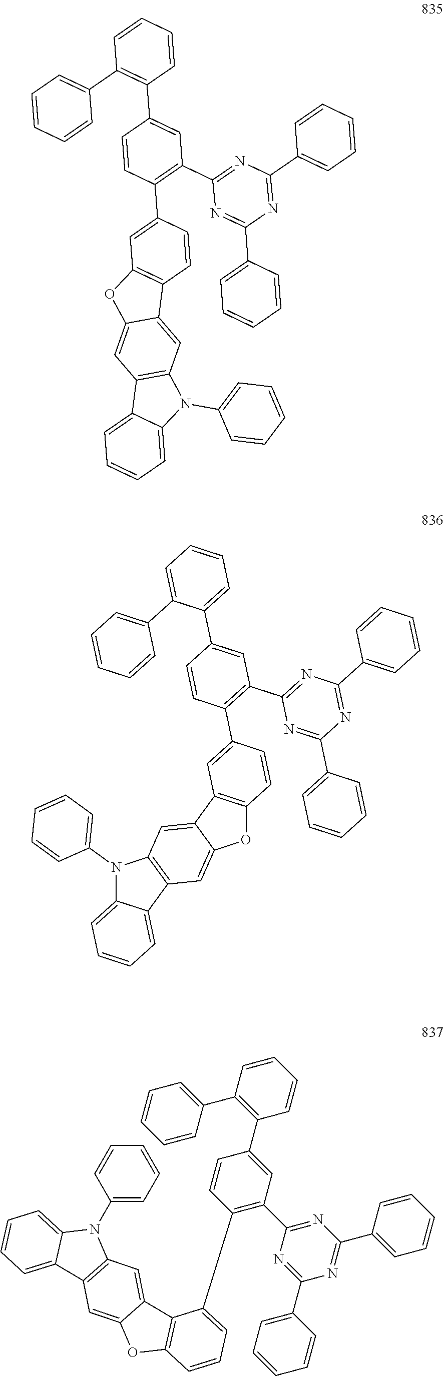 [SMILES: c1ccc(-c2nc(-c3ccccc3)nc(-c3cc(-c4ccccc4-c4ccccc4)ccc3-c3ccc4c(c3)oc3cc5c6ccccc6n(-c6ccccc6)c5cc34)n2)cc1 ; c1ccc(-c2nc(-c3ccccc3)nc(-c3cc(-c4ccccc4-c4ccccc4)ccc3-c3ccc4oc5cc6c7ccccc7n(-c7ccccc7)c6cc5c4c3)n2)cc1 ; c1ccc(-c2nc(-c3ccccc3)nc(-c3cc(-c4ccccc4-c4ccccc4)ccc3-c3cccc4oc5cc6c7ccccc7n(-c7ccccc7)c6cc5c34)n2)cc1]

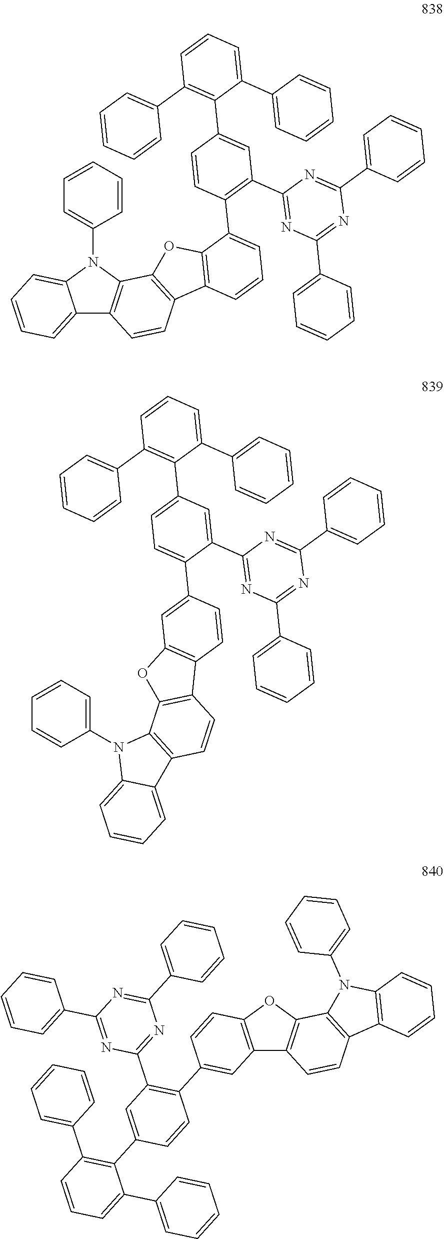 [SMILES: c1ccc(-c2nc(-c3ccccc3)nc(-c3cc(-c4c(-c5ccccc5)cccc4-c4ccccc4)ccc3-c3cccc4c3oc3c4ccc4c5ccccc5n(-c5ccccc5)c43)n2)cc1 ; c1ccc(-c2nc(-c3ccccc3)nc(-c3cc(-c4c(-c5ccccc5)cccc4-c4ccccc4)ccc3-c3ccc4c(c3)oc3c4ccc4c5ccccc5n(-c5ccccc5)c43)n2)cc1 ; c1ccc(-c2nc(-c3ccccc3)nc(-c3cc(-c4c(-c5ccccc5)cccc4-c4ccccc4)ccc3-c3ccc4oc5c(ccc6c7ccccc7n(-c7ccccc7)c65)c4c3)n2)cc1]

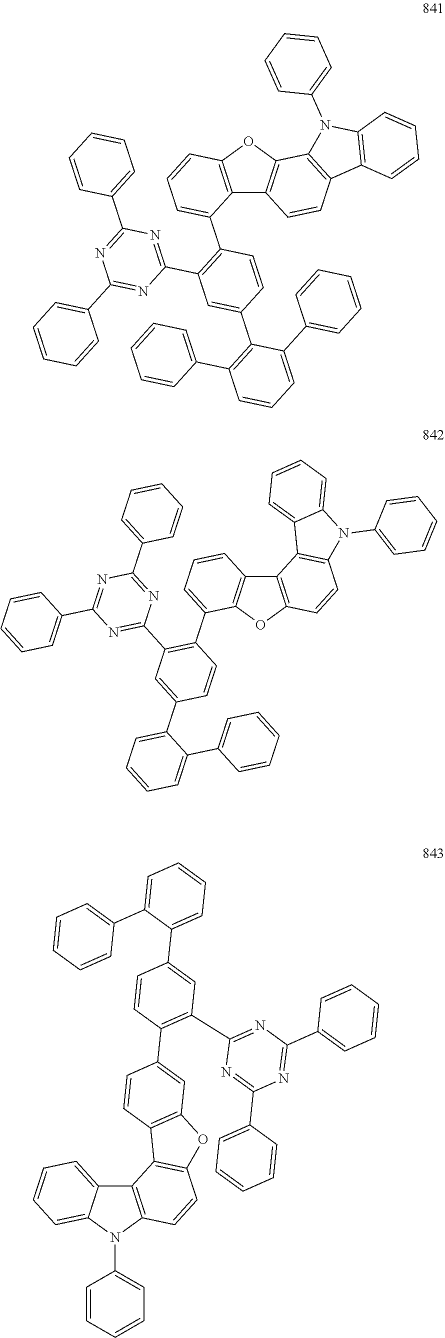 [SMILES: c1ccc(-c2nc(-c3ccccc3)nc(-c3cc(-c4c(-c5ccccc5)cccc4-c4ccccc4)ccc3-c3cccc4oc5c(ccc6c7ccccc7n(-c7ccccc7)c65)c34)n2)cc1 ; c1ccc(-c2nc(-c3ccccc3)nc(-c3cc(-c4ccccc4-c4ccccc4)ccc3-c3cccc4c3oc3ccc5c(c6ccccc6n5-c5ccccc5)c34)n2)cc1 ; c1ccc(-c2nc(-c3ccccc3)nc(-c3cc(-c4ccccc4-c4ccccc4)ccc3-c3ccc4c(c3)oc3ccc5c(c6ccccc6n5-c5ccccc5)c34)n2)cc1]

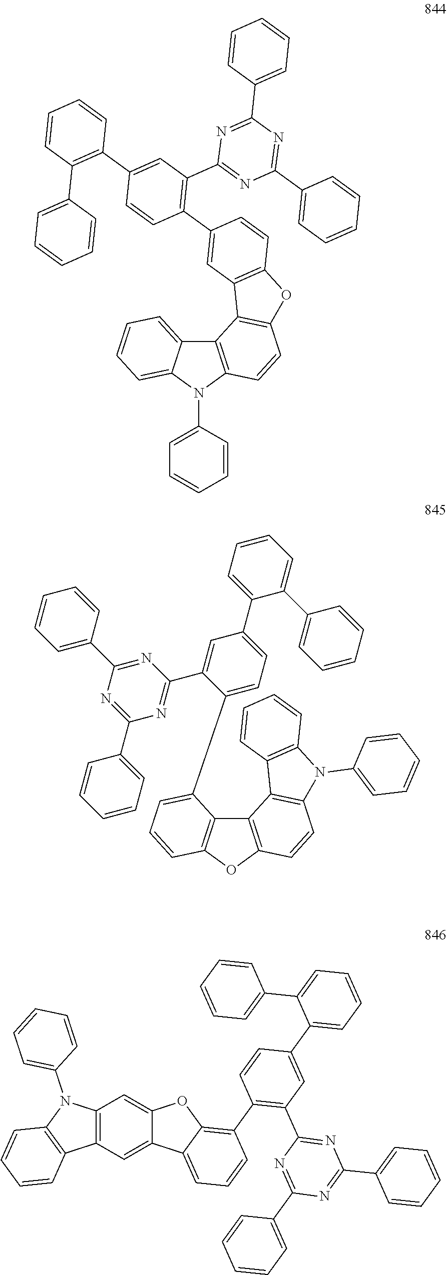 [SMILES: c1ccc(-c2nc(-c3ccccc3)nc(-c3cc(-c4ccccc4-c4ccccc4)ccc3-c3ccc4oc5ccc6c(c7ccccc7n6-c6ccccc6)c5c4c3)n2)cc1 ; c1ccc(-c2nc(-c3ccccc3)nc(-c3cc(-c4ccccc4-c4ccccc4)ccc3-c3cccc4oc5ccc6c(c7ccccc7n6-c6ccccc6)c5c34)n2)cc1 ; c1ccc(-c2nc(-c3ccccc3)nc(-c3cc(-c4ccccc4-c4ccccc4)ccc3-c3cccc4c3oc3cc5c(cc34)c3ccccc3n5-c3ccccc3)n2)cc1]

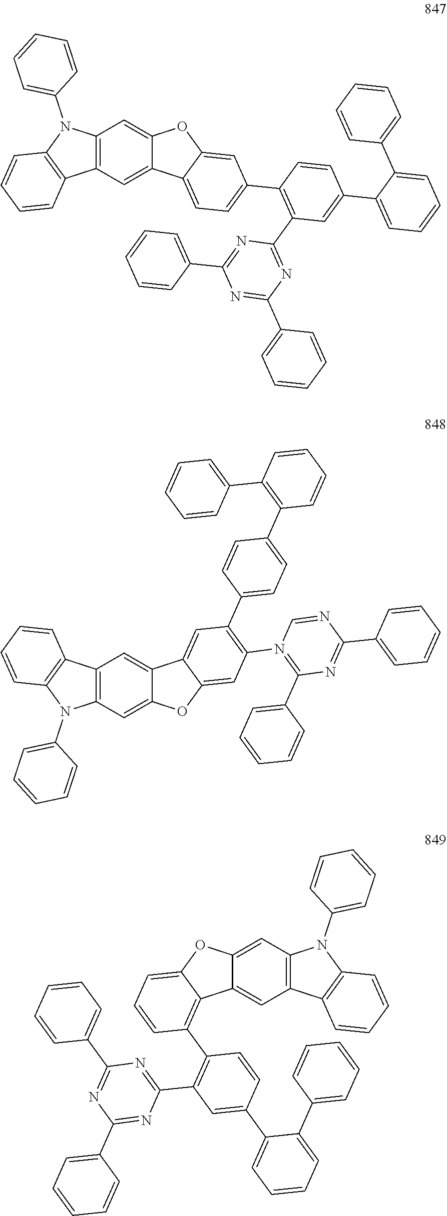

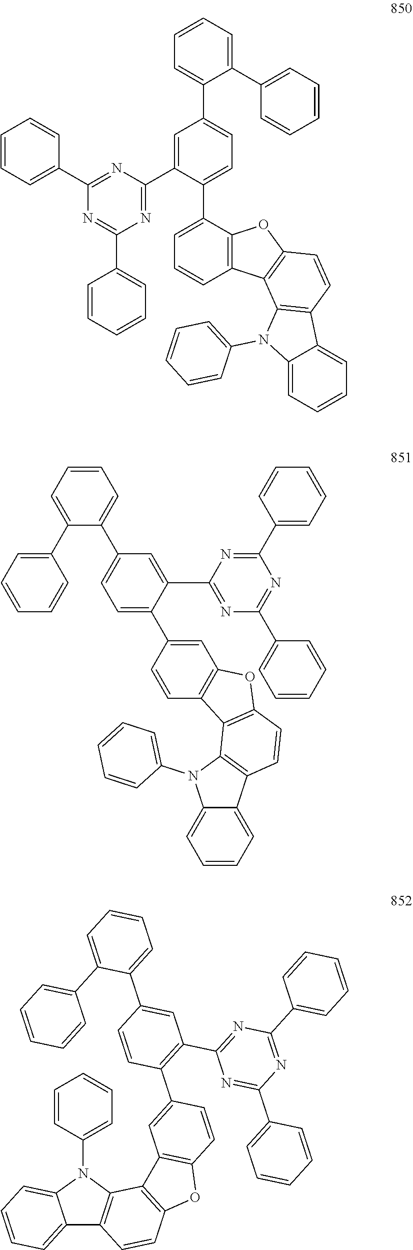 [SMILES: c1ccc(-c2nc(-c3ccccc3)nc(-c3cc(-c4ccccc4-c4ccccc4)ccc3-c3cccc4c3oc3ccc5c6ccccc6n(-c6ccccc6)c5c34)n2)cc1 ; c1ccc(-c2nc(-c3ccccc3)nc(-c3cc(-c4ccccc4-c4ccccc4)ccc3-c3ccc4c(c3)oc3ccc5c6ccccc6n(-c6ccccc6)c5c34)n2)cc1 ; c1ccc(-c2nc(-c3ccccc3)nc(-c3cc(-c4ccccc4-c4ccccc4)ccc3-c3ccc4oc5ccc6c7ccccc7n(-c7ccccc7)c6c5c4c3)n2)cc1]

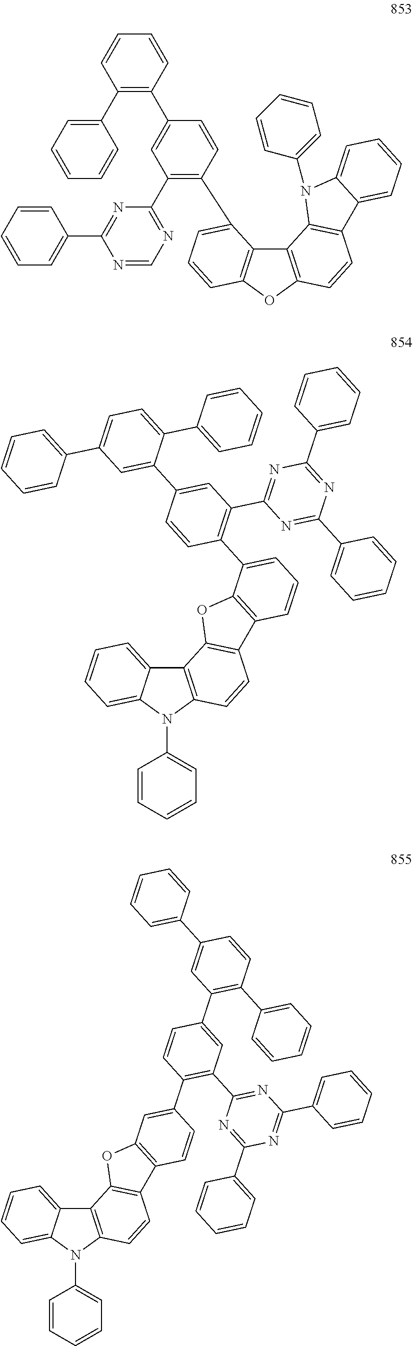 [SMILES: c1ccc(-c2ncnc(-c3cc(-c4ccccc4-c4ccccc4)ccc3-c3cccc4oc5ccc6c7ccccc7n(-c7ccccc7)c6c5c34)n2)cc1 ; c1ccc(-c2ccc(-c3ccccc3)c(-c3ccc(-c4cccc5c4oc4c5ccc5c4c4ccccc4n5-c4ccccc4)c(-c4nc(-c5ccccc5)nc(-c5ccccc5)n4)c3)c2)cc1 ; c1ccc(-c2ccc(-c3ccccc3)c(-c3ccc(-c4ccc5c(c4)oc4c5ccc5c4c4ccccc4n5-c4ccccc4)c(-c4nc(-c5ccccc5)nc(-c5ccccc5)n4)c3)c2)cc1]

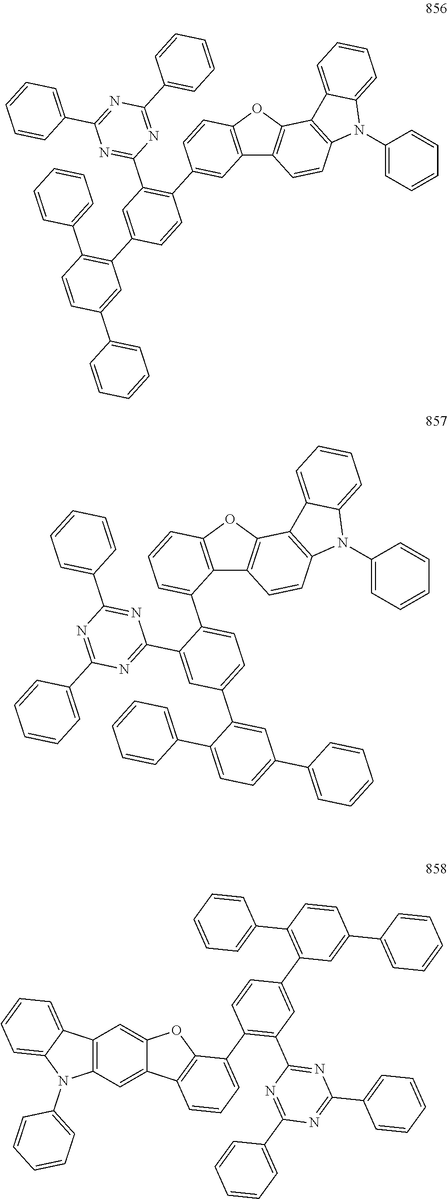 [SMILES: c1ccc(-c2ccc(-c3ccccc3)c(-c3ccc(-c4ccc5oc6c(ccc7c6c6ccccc6n7-c6ccccc6)c5c4)c(-c4nc(-c5ccccc5)nc(-c5ccccc5)n4)c3)c2)cc1 ; c1ccc(-c2ccc(-c3ccccc3)c(-c3ccc(-c4cccc5oc6c(ccc7c6c6ccccc6n7-c6ccccc6)c45)c(-c4nc(-c5ccccc5)nc(-c5ccccc5)n4)c3)c2)cc1 ; c1ccc(-c2ccc(-c3ccccc3)c(-c3ccc(-c4cccc5c4oc4cc6c7ccccc7n(-c7ccccc7)c6cc45)c(-c4nc(-c5ccccc5)nc(-c5ccccc5)n4)c3)c2)cc1]

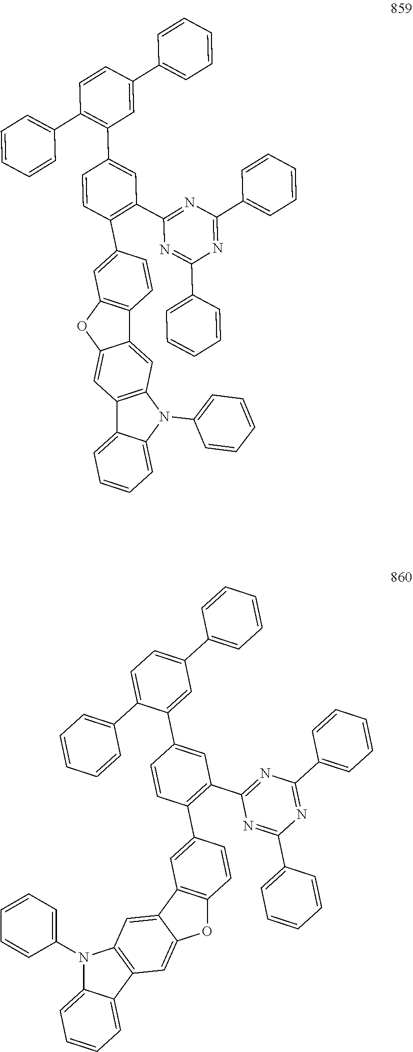 [SMILES: c1ccc(-c2ccc(-c3ccccc3)c(-c3ccc(-c4ccc5c(c4)oc4cc6c7ccccc7n(-c7ccccc7)c6cc45)c(-c4nc(-c5ccccc5)nc(-c5ccccc5)n4)c3)c2)cc1 ; c1ccc(-c2ccc(-c3ccccc3)c(-c3ccc(-c4ccc5oc6cc7c8ccccc8n(-c8ccccc8)c7cc6c5c4)c(-c4nc(-c5ccccc5)nc(-c5ccccc5)n4)c3)c2)cc1]

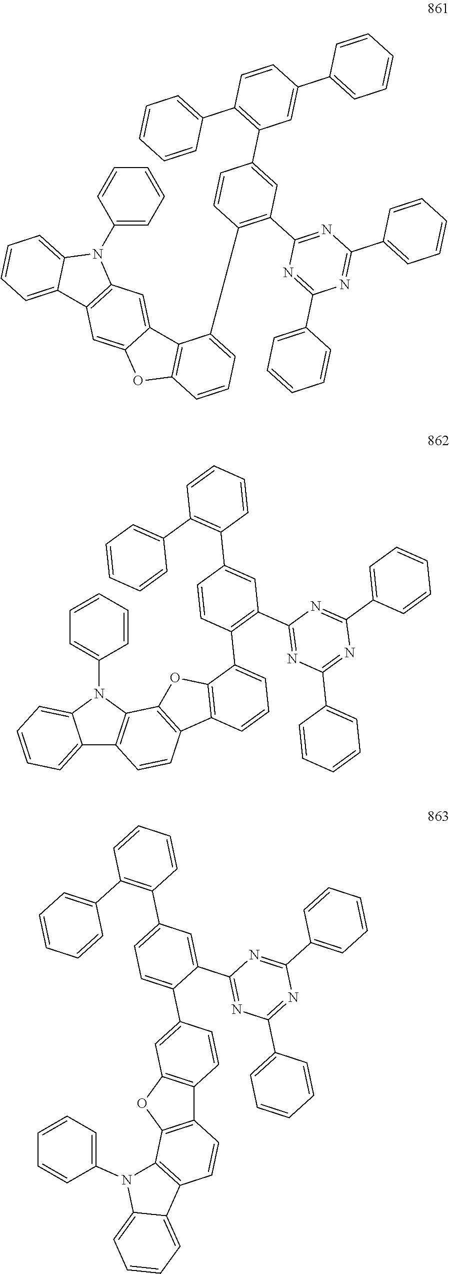 [SMILES: c1ccc(-c2ccc(-c3ccccc3)c(-c3ccc(-c4cccc5oc6cc7c8ccccc8n(-c8ccccc8)c7cc6c45)c(-c4nc(-c5ccccc5)nc(-c5ccccc5)n4)c3)c2)cc1 ; c1ccc(-c2nc(-c3ccccc3)nc(-c3cc(-c4ccccc4-c4ccccc4)ccc3-c3cccc4c3oc3c4ccc4c5ccccc5n(-c5ccccc5)c43)n2)cc1 ; c1ccc(-c2nc(-c3ccccc3)nc(-c3cc(-c4ccccc4-c4ccccc4)ccc3-c3ccc4c(c3)oc3c4ccc4c5ccccc5n(-c5ccccc5)c43)n2)cc1]

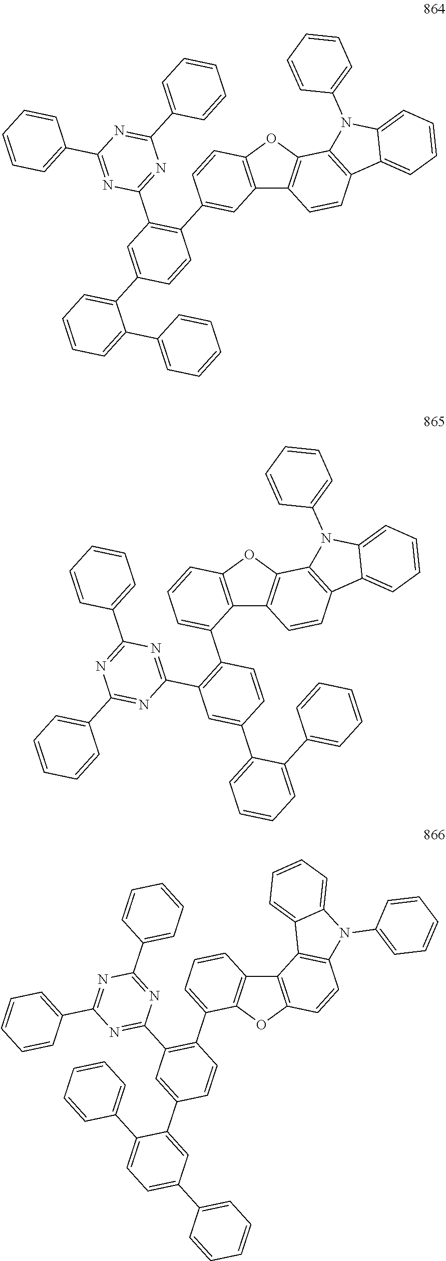 [SMILES: c1ccc(-c2nc(-c3ccccc3)nc(-c3cc(-c4ccccc4-c4ccccc4)ccc3-c3ccc4oc5c(ccc6c7ccccc7n(-c7ccccc7)c65)c4c3)n2)cc1 ; c1ccc(-c2nc(-c3ccccc3)nc(-c3cc(-c4ccccc4-c4ccccc4)ccc3-c3cccc4oc5c(ccc6c7ccccc7n(-c7ccccc7)c65)c34)n2)cc1 ; c1ccc(-c2ccc(-c3ccccc3)c(-c3ccc(-c4cccc5c4oc4ccc6c(c7ccccc7n6-c6ccccc6)c45)c(-c4nc(-c5ccccc5)nc(-c5ccccc5)n4)c3)c2)cc1]

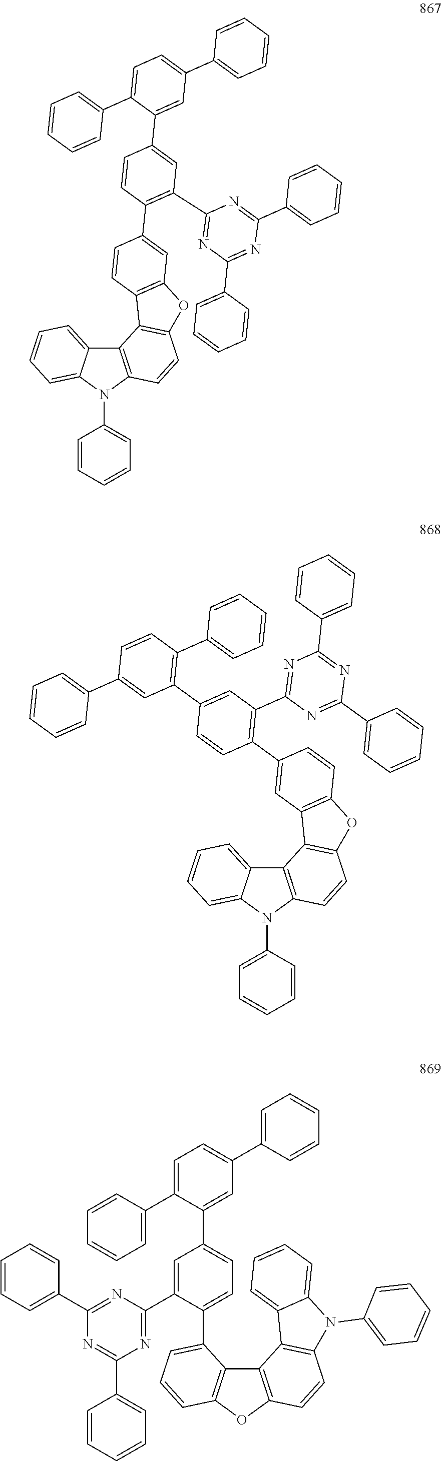 [SMILES: c1ccc(-c2ccc(-c3ccccc3)c(-c3ccc(-c4ccc5c(c4)oc4ccc6c(c7ccccc7n6-c6ccccc6)c45)c(-c4nc(-c5ccccc5)nc(-c5ccccc5)n4)c3)c2)cc1 ; c1ccc(-c2ccc(-c3ccccc3)c(-c3ccc(-c4ccc5oc6ccc7c(c8ccccc8n7-c7ccccc7)c6c5c4)c(-c4nc(-c5ccccc5)nc(-c5ccccc5)n4)c3)c2)cc1 ; c1ccc(-c2ccc(-c3ccccc3)c(-c3ccc(-c4cccc5oc6ccc7c(c8ccccc8n7-c7ccccc7)c6c45)c(-c4nc(-c5ccccc5)nc(-c5ccccc5)n4)c3)c2)cc1]

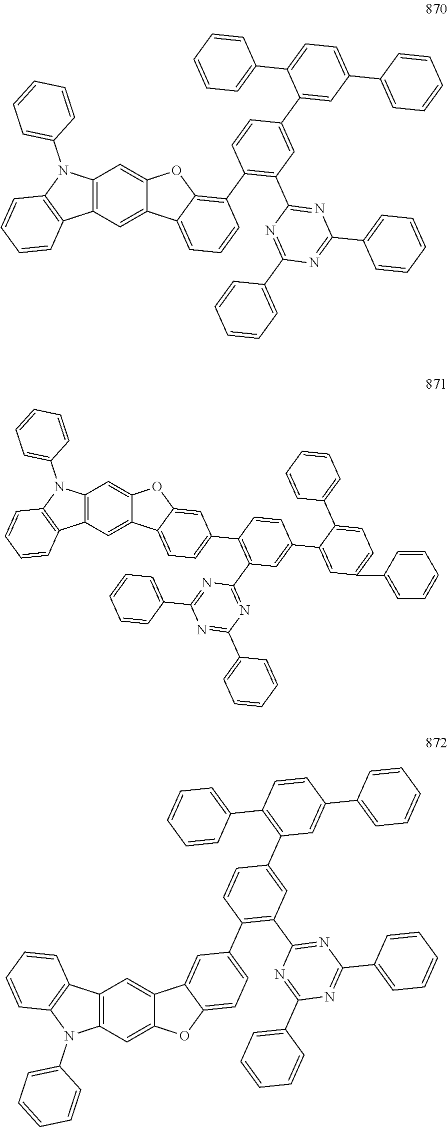 [SMILES: c1ccc(-c2ccc(-c3ccccc3)c(-c3ccc(-c4cccc5c4oc4cc6c(cc45)c4ccccc4n6-c4ccccc4)c(-c4nc(-c5ccccc5)nc(-c5ccccc5)n4)c3)c2)cc1 ; c1ccc(-c2ccc(-c3ccccc3)c(-c3ccc(-c4ccc5c(c4)oc4cc6c(cc45)c4ccccc4n6-c4ccccc4)c(-c4nc(-c5ccccc5)nc(-c5ccccc5)n4)c3)c2)cc1 ; c1ccc(-c2ccc(-c3ccccc3)c(-c3ccc(-c4ccc5oc6cc7c(cc6c5c4)c4ccccc4n7-c4ccccc4)c(-c4nc(-c5ccccc5)nc(-c5ccccc5)n4)c3)c2)cc1]

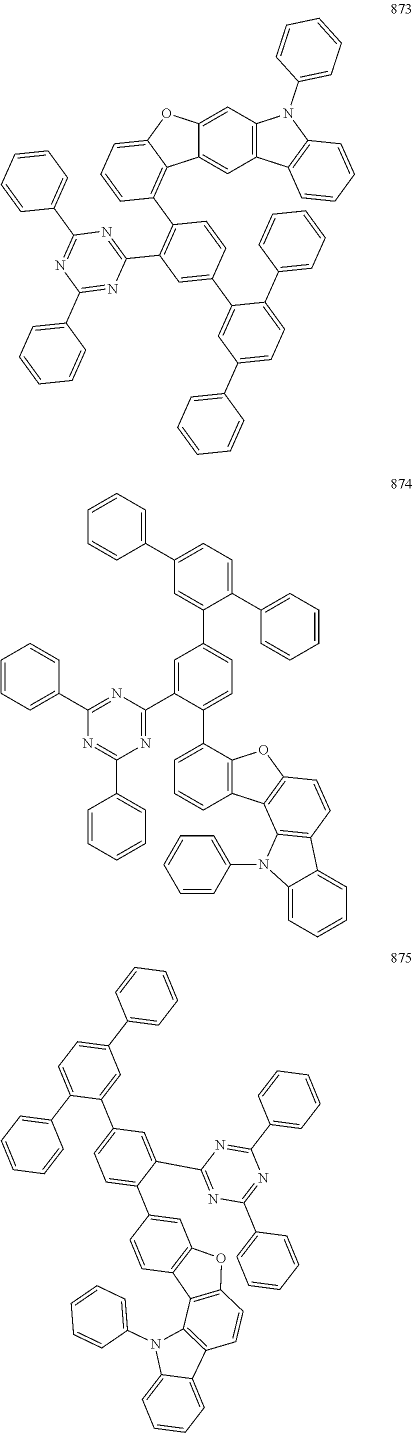 [SMILES: c1ccc(-c2ccc(-c3ccccc3)c(-c3ccc(-c4cccc5oc6cc7c(cc6c45)c4ccccc4n7-c4ccccc4)c(-c4nc(-c5ccccc5)nc(-c5ccccc5)n4)c3)c2)cc1 ; c1ccc(-c2ccc(-c3ccccc3)c(-c3ccc(-c4cccc5c4oc4ccc6c7ccccc7n(-c7ccccc7)c6c45)c(-c4nc(-c5ccccc5)nc(-c5ccccc5)n4)c3)c2)cc1 ; c1ccc(-c2ccc(-c3ccccc3)c(-c3ccc(-c4ccc5c(c4)oc4ccc6c7ccccc7n(-c7ccccc7)c6c45)c(-c4nc(-c5ccccc5)nc(-c5ccccc5)n4)c3)c2)cc1]

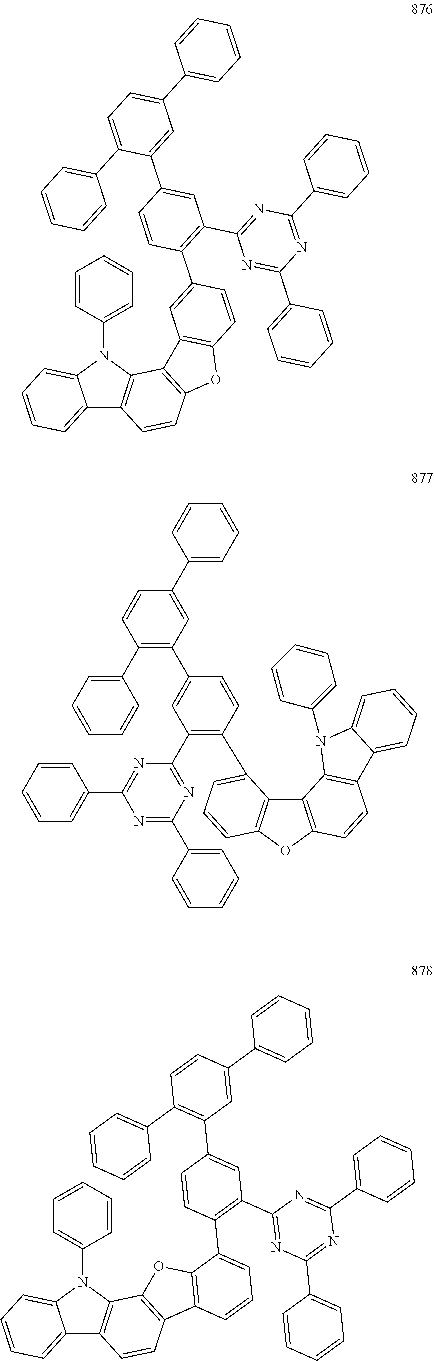 [SMILES: c1ccc(-c2ccc(-c3ccccc3)c(-c3ccc(-c4ccc5oc6ccc7c8ccccc8n(-c8ccccc8)c7c6c5c4)c(-c4nc(-c5ccccc5)nc(-c5ccccc5)n4)c3)c2)cc1 ; c1ccc(-c2ccc(-c3ccccc3)c(-c3ccc(-c4cccc5oc6ccc7c8ccccc8n(-c8ccccc8)c7c6c45)c(-c4nc(-c5ccccc5)nc(-c5ccccc5)n4)c3)c2)cc1 ; c1ccc(-c2ccc(-c3ccccc3)c(-c3ccc(-c4cccc5c4oc4c5ccc5c6ccccc6n(-c6ccccc6)c54)c(-c4nc(-c5ccccc5)nc(-c5ccccc5)n4)c3)c2)cc1]

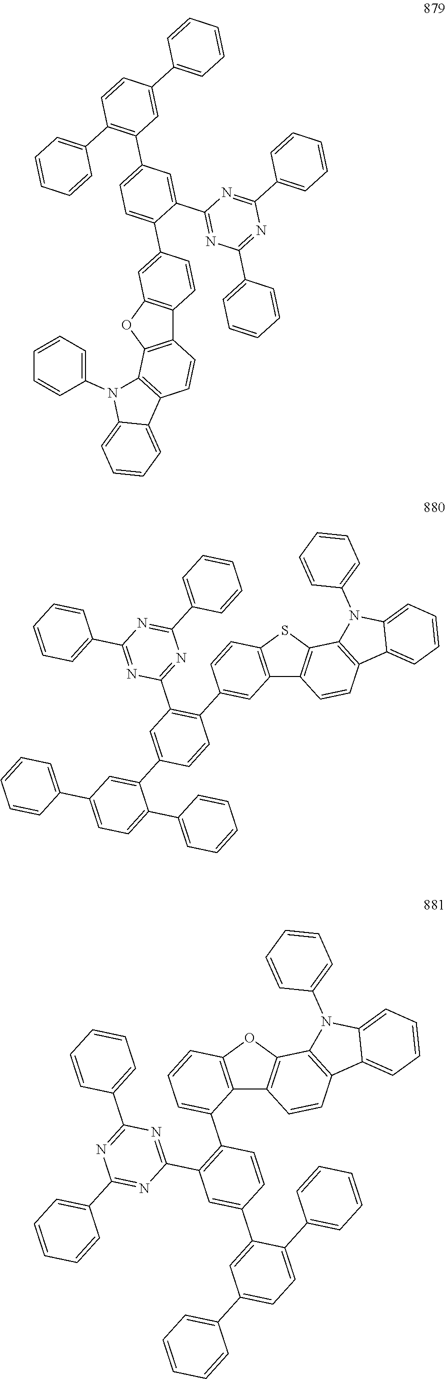 [SMILES: c1ccc(-c2ccc(-c3ccccc3)c(-c3ccc(-c4ccc5c(c4)oc4c5ccc5c6ccccc6n(-c6ccccc6)c54)c(-c4nc(-c5ccccc5)nc(-c5ccccc5)n4)c3)c2)cc1 ; c1ccc(-c2ccc(-c3ccccc3)c(-c3ccc(-c4ccc5sc6c(ccc7c8ccccc8n(-c8ccccc8)c76)c5c4)c(-c4nc(-c5ccccc5)nc(-c5ccccc5)n4)c3)c2)cc1 ; c1ccc(-c2ccc(-c3ccccc3)c(-c3ccc(-c4cccc5oc6c(ccc7c8ccccc8n(-c8ccccc8)c76)c45)c(-c4nc(-c5ccccc5)nc(-c5ccccc5)n4)c3)c2)cc1]

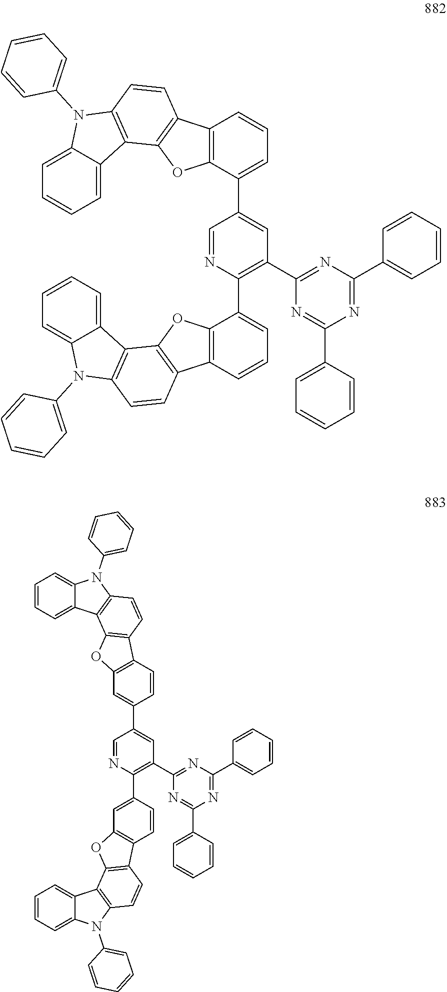 [SMILES: c1ccc(-c2nc(-c3ccccc3)nc(-c3cc(-c4cccc5c4oc4c5ccc5c4c4ccccc4n5-c4ccccc4)cnc3-c3cccc4c3oc3c4ccc4c3c3ccccc3n4-c3ccccc3)n2)cc1 ; c1ccc(-c2nc(-c3ccccc3)nc(-c3cc(-c4ccc5c(c4)oc4c5ccc5c4c4ccccc4n5-c4ccccc4)cnc3-c3ccc4c(c3)oc3c4ccc4c3c3ccccc3n4-c3ccccc3)n2)cc1]

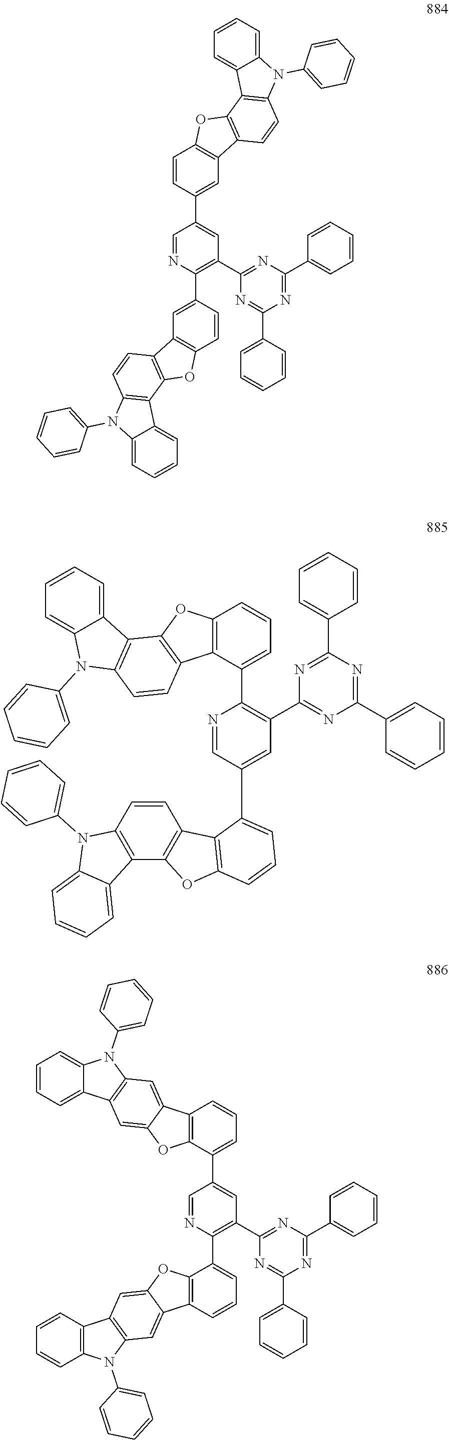 [SMILES: c1ccc(-c2nc(-c3ccccc3)nc(-c3cc(-c4ccc5oc6c(ccc7c6c6ccccc6n7-c6ccccc6)c5c4)cnc3-c3ccc4oc5c(ccc6c5c5ccccc5n6-c5ccccc5)c4c3)n2)cc1 ; c1ccc(-c2nc(-c3ccccc3)nc(-c3cc(-c4cccc5oc6c(ccc7c6c6ccccc6n7-c6ccccc6)c45)cnc3-c3cccc4oc5c(ccc6c5c5ccccc5n6-c5ccccc5)c34)n2)cc1 ; c1ccc(-c2nc(-c3ccccc3)nc(-c3cc(-c4cccc5c4oc4cc6c7ccccc7n(-c7ccccc7)c6cc45)cnc3-c3cccc4c3oc3cc5c6ccccc6n(-c6ccccc6)c5cc34)n2)cc1]

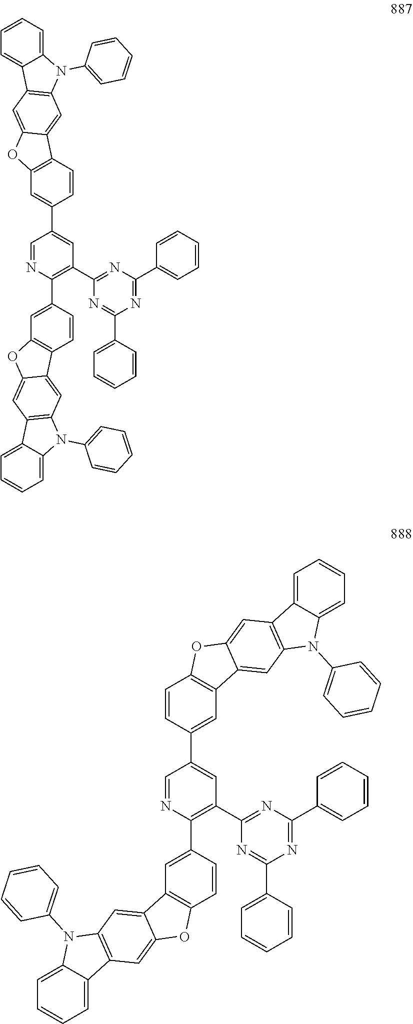 [SMILES: c1ccc(-c2nc(-c3ccccc3)nc(-c3cc(-c4ccc5c(c4)oc4cc6c7ccccc7n(-c7ccccc7)c6cc45)cnc3-c3ccc4c(c3)oc3cc5c6ccccc6n(-c6ccccc6)c5cc34)n2)cc1 ; c1ccc(-c2nc(-c3ccccc3)nc(-c3cc(-c4ccc5oc6cc7c8ccccc8n(-c8ccccc8)c7cc6c5c4)cnc3-c3ccc4oc5cc6c7ccccc7n(-c7ccccc7)c6cc5c4c3)n2)cc1]

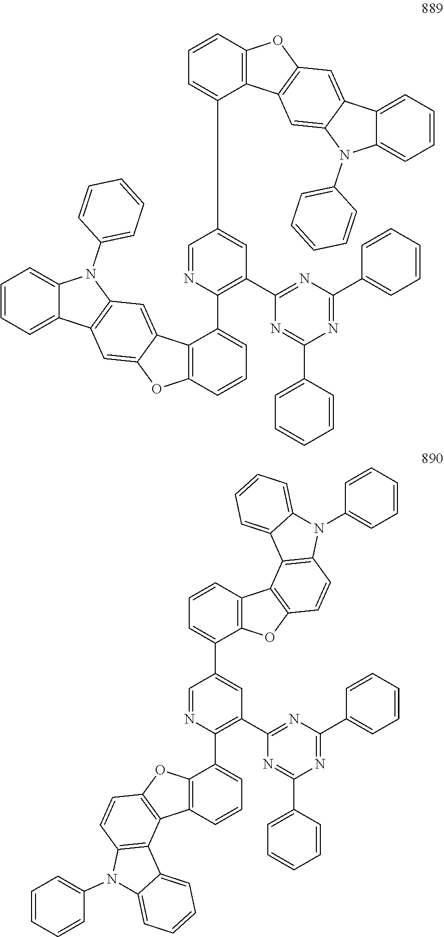

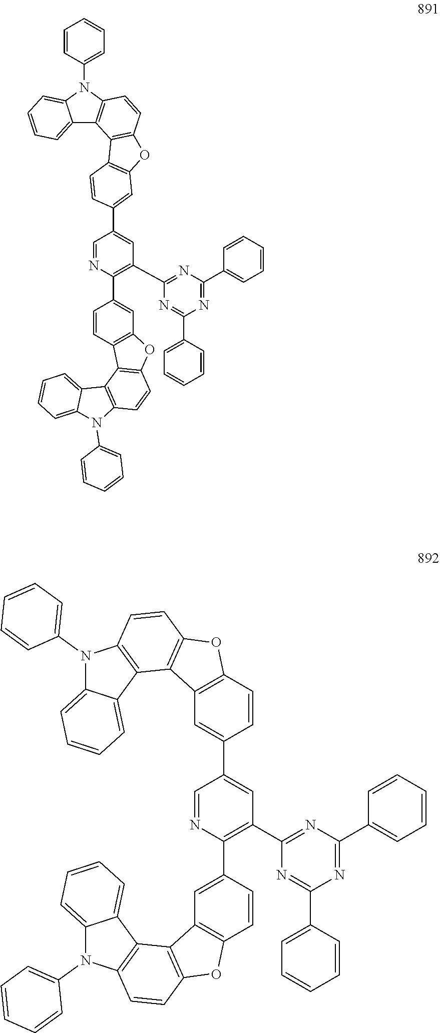 [SMILES: c1ccc(-c2nc(-c3ccccc3)nc(-c3cc(-c4ccc5c(c4)oc4ccc6c(c7ccccc7n6-c6ccccc6)c45)cnc3-c3ccc4c(c3)oc3ccc5c(c6ccccc6n5-c5ccccc5)c34)n2)cc1 ; c1ccc(-c2nc(-c3ccccc3)nc(-c3cc(-c4ccc5oc6ccc7c(c8ccccc8n7-c7ccccc7)c6c5c4)cnc3-c3ccc4oc5ccc6c(c7ccccc7n6-c6ccccc6)c5c4c3)n2)cc1]

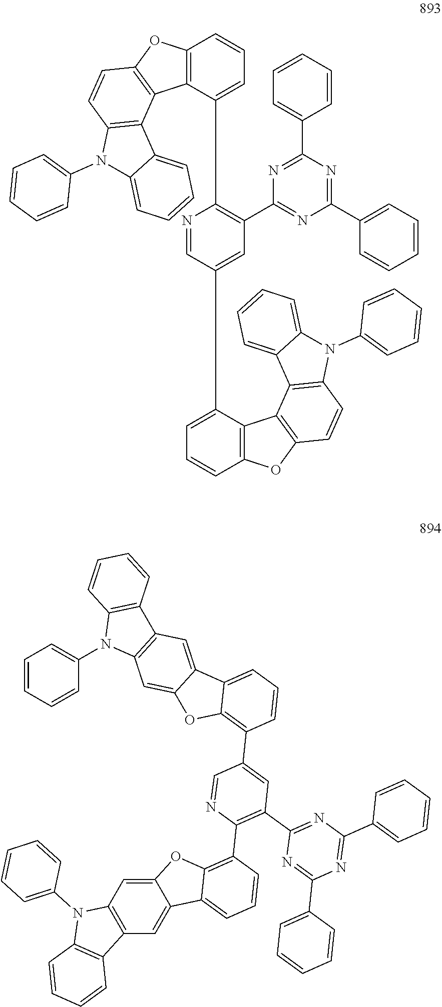 [SMILES: c1ccc(-c2nc(-c3ccccc3)nc(-c3cc(-c4cccc5oc6ccc7c(c8ccccc8n7-c7ccccc7)c6c45)cnc3-c3cccc4oc5ccc6c(c7ccccc7n6-c6ccccc6)c5c34)n2)cc1 ; c1ccc(-c2nc(-c3ccccc3)nc(-c3cc(-c4cccc5c4oc4cc6c(cc45)c4ccccc4n6-c4ccccc4)cnc3-c3cccc4c3oc3cc5c(cc34)c3ccccc3n5-c3ccccc3)n2)cc1]

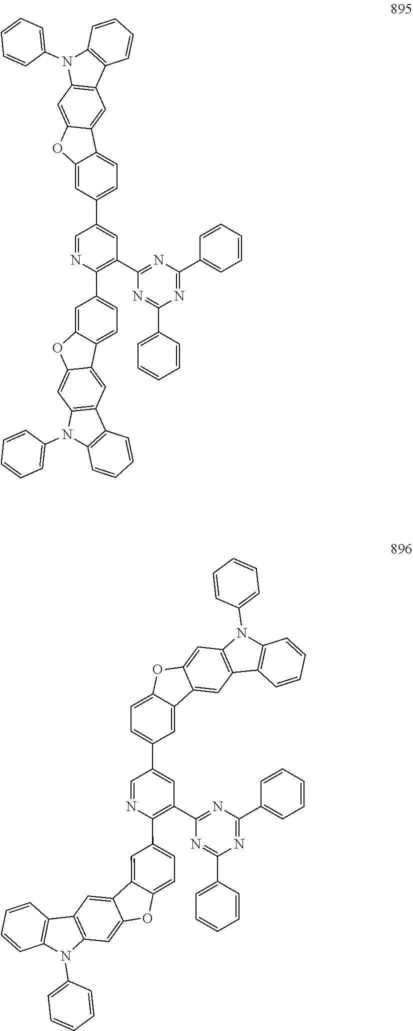 [SMILES: c1ccc(-c2nc(-c3ccccc3)nc(-c3cc(-c4ccc5c(c4)oc4cc6c(cc45)c4ccccc4n6-c4ccccc4)cnc3-c3ccc4c(c3)oc3cc5c(cc34)c3ccccc3n5-c3ccccc3)n2)cc1 ; c1ccc(-c2nc(-c3ccccc3)nc(-c3cc(-c4ccc5oc6cc7c(cc6c5c4)c4ccccc4n7-c4ccccc4)cnc3-c3ccc4oc5cc6c(cc5c4c3)c3ccccc3n6-c3ccccc3)n2)cc1]

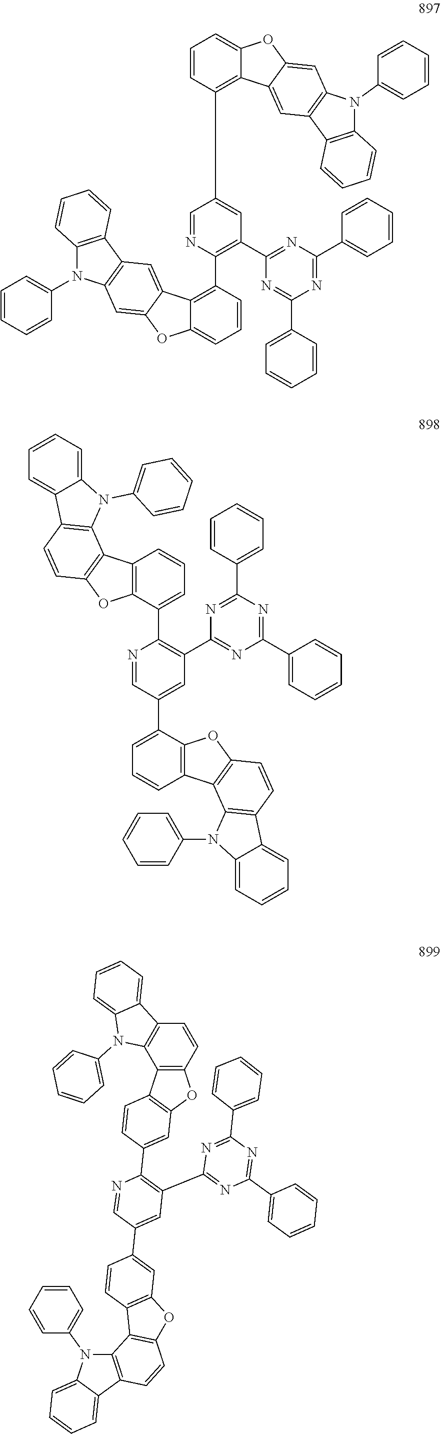 [SMILES: c1ccc(-c2nc(-c3ccccc3)nc(-c3cc(-c4cccc5oc6cc7c(cc6c45)c4ccccc4n7-c4ccccc4)cnc3-c3cccc4oc5cc6c(cc5c34)c3ccccc3n6-c3ccccc3)n2)cc1 ; c1ccc(-c2nc(-c3ccccc3)nc(-c3cc(-c4cccc5c4oc4ccc6c7ccccc7n(-c7ccccc7)c6c45)cnc3-c3cccc4c3oc3ccc5c6ccccc6n(-c6ccccc6)c5c34)n2)cc1 ; c1ccc(-c2nc(-c3ccccc3)nc(-c3cc(-c4ccc5c(c4)oc4ccc6c7ccccc7n(-c7ccccc7)c6c45)cnc3-c3ccc4c(c3)oc3ccc5c6ccccc6n(-c6ccccc6)c5c34)n2)cc1]

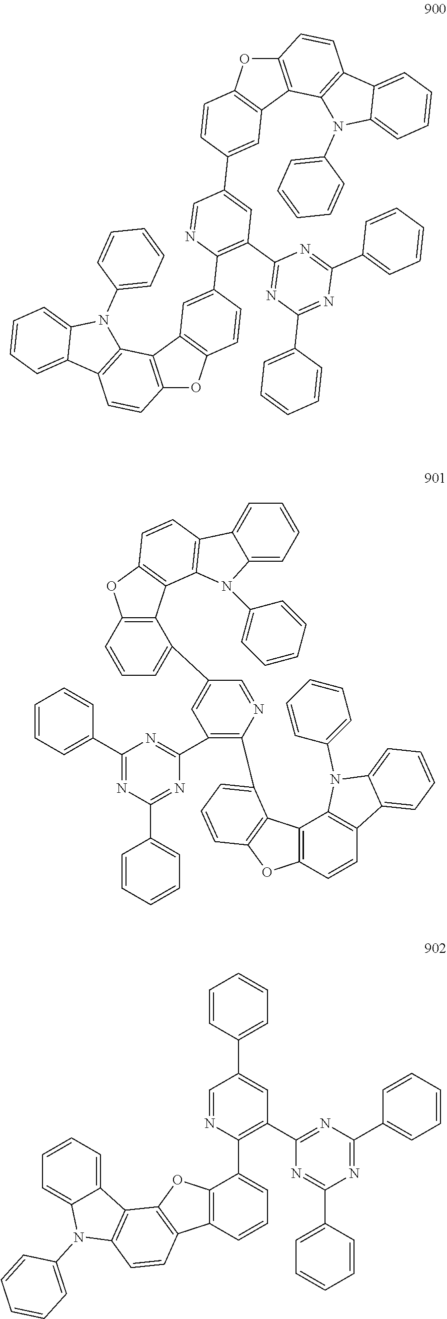 [SMILES: c1ccc(-c2nc(-c3ccccc3)nc(-c3cc(-c4ccc5oc6ccc7c8ccccc8n(-c8ccccc8)c7c6c5c4)cnc3-c3ccc4oc5ccc6c7ccccc7n(-c7ccccc7)c6c5c4c3)n2)cc1 ; c1ccc(-c2nc(-c3ccccc3)nc(-c3cc(-c4cccc5oc6ccc7c8ccccc8n(-c8ccccc8)c7c6c45)cnc3-c3cccc4oc5ccc6c7ccccc7n(-c7ccccc7)c6c5c34)n2)cc1 ; c1ccc(-c2cnc(-c3cccc4c3oc3c4ccc4c3c3ccccc3n4-c3ccccc3)c(-c3nc(-c4ccccc4)nc(-c4ccccc4)n3)c2)cc1]

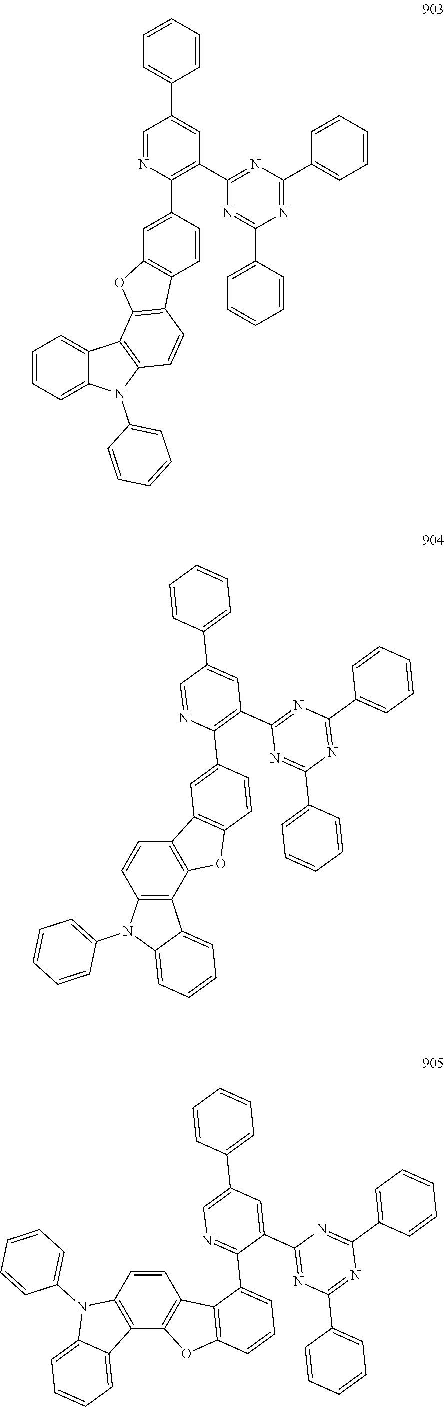 [SMILES: c1ccc(-c2cnc(-c3ccc4c(c3)oc3c4ccc4c3c3ccccc3n4-c3ccccc3)c(-c3nc(-c4ccccc4)nc(-c4ccccc4)n3)c2)cc1 ; c1ccc(-c2cnc(-c3ccc4oc5c(ccc6c5c5ccccc5n6-c5ccccc5)c4c3)c(-c3nc(-c4ccccc4)nc(-c4ccccc4)n3)c2)cc1 ; c1ccc(-c2cnc(-c3cccc4oc5c(ccc6c5c5ccccc5n6-c5ccccc5)c34)c(-c3nc(-c4ccccc4)nc(-c4ccccc4)n3)c2)cc1]

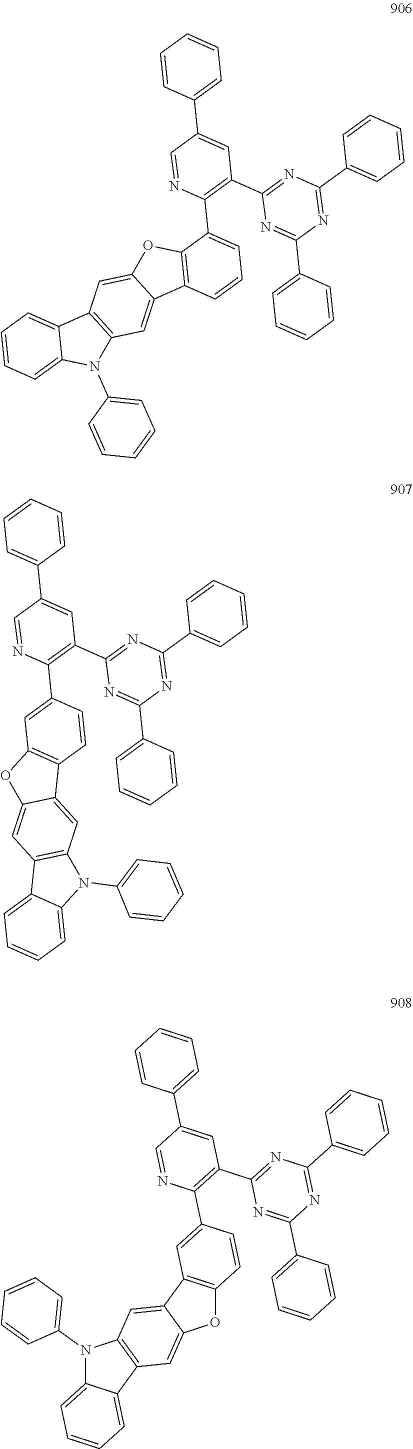 [SMILES: c1ccc(-c2cnc(-c3cccc4c3oc3cc5c6ccccc6n(-c6ccccc6)c5cc34)c(-c3nc(-c4ccccc4)nc(-c4ccccc4)n3)c2)cc1 ; c1ccc(-c2cnc(-c3ccc4c(c3)oc3cc5c6ccccc6n(-c6ccccc6)c5cc34)c(-c3nc(-c4ccccc4)nc(-c4ccccc4)n3)c2)cc1 ; c1ccc(-c2cnc(-c3ccc4oc5cc6c7ccccc7n(-c7ccccc7)c6cc5c4c3)c(-c3nc(-c4ccccc4)nc(-c4ccccc4)n3)c2)cc1]

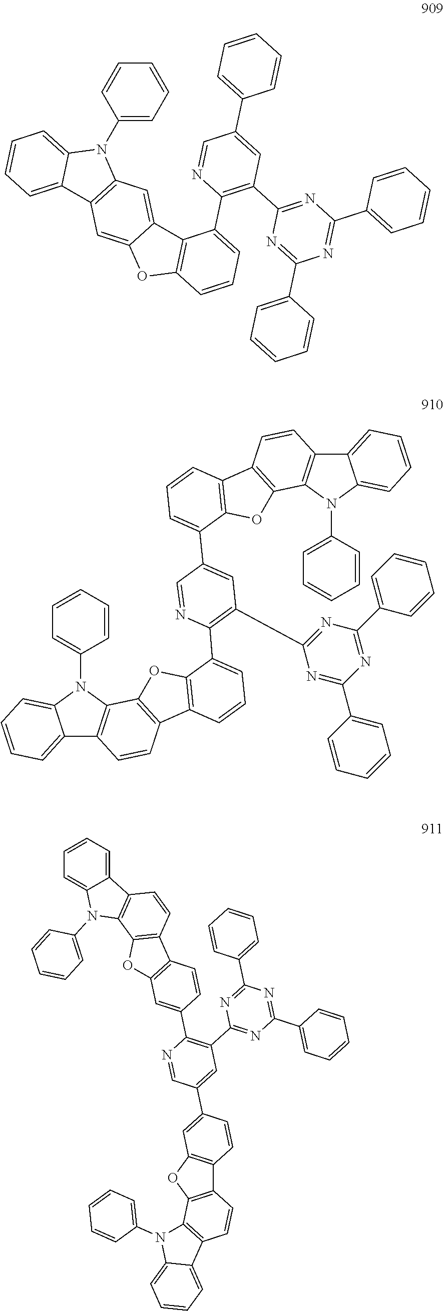 [SMILES: c1ccc(-c2cnc(-c3cccc4oc5cc6c7ccccc7n(-c7ccccc7)c6cc5c34)c(-c3nc(-c4ccccc4)nc(-c4ccccc4)n3)c2)cc1 ; c1ccc(-c2nc(-c3ccccc3)nc(-c3cc(-c4cccc5c4oc4c5ccc5c6ccccc6n(-c6ccccc6)c54)cnc3-c3cccc4c3oc3c4ccc4c5ccccc5n(-c5ccccc5)c43)n2)cc1 ; c1ccc(-c2nc(-c3ccccc3)nc(-c3cc(-c4ccc5c(c4)oc4c5ccc5c6ccccc6n(-c6ccccc6)c54)cnc3-c3ccc4c(c3)oc3c4ccc4c5ccccc5n(-c5ccccc5)c43)n2)cc1]

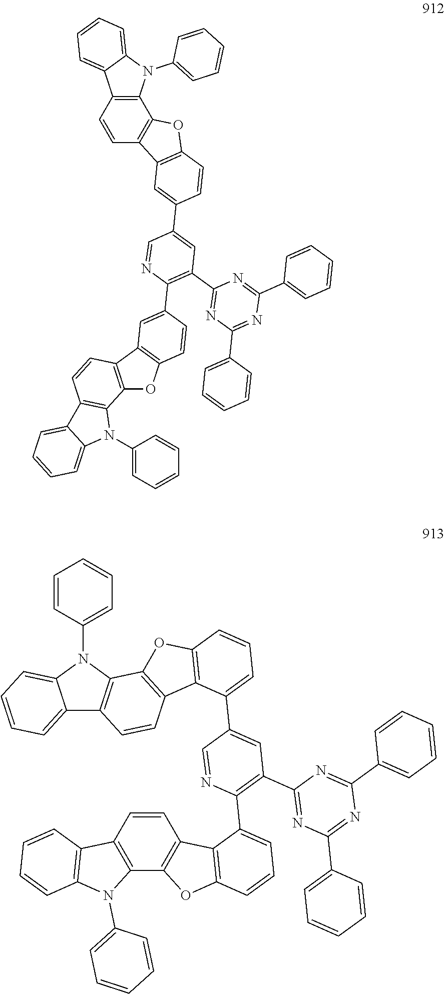 [SMILES: c1ccc(-c2nc(-c3ccccc3)nc(-c3cc(-c4ccc5oc6c(ccc7c8ccccc8n(-c8ccccc8)c76)c5c4)cnc3-c3ccc4oc5c(ccc6c7ccccc7n(-c7ccccc7)c65)c4c3)n2)cc1 ; c1ccc(-c2nc(-c3ccccc3)nc(-c3cc(-c4cccc5oc6c(ccc7c8ccccc8n(-c8ccccc8)c76)c45)cnc3-c3cccc4oc5c(ccc6c7ccccc7n(-c7ccccc7)c65)c34)n2)cc1]

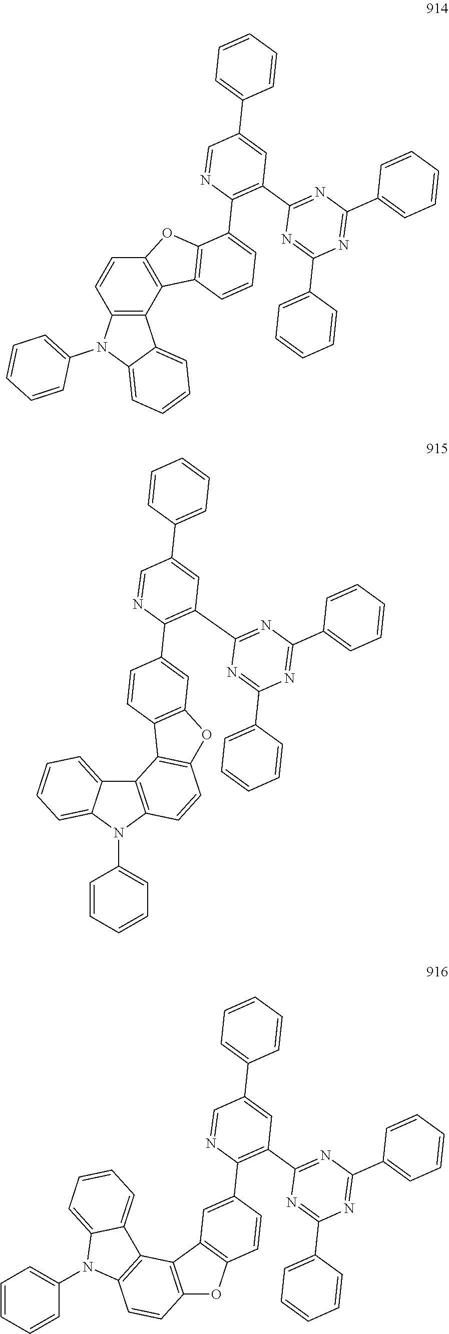 [SMILES: c1ccc(-c2cnc(-c3cccc4c3oc3ccc5c(c6ccccc6n5-c5ccccc5)c34)c(-c3nc(-c4ccccc4)nc(-c4ccccc4)n3)c2)cc1 ; c1ccc(-c2cnc(-c3ccc4c(c3)oc3ccc5c(c6ccccc6n5-c5ccccc5)c34)c(-c3nc(-c4ccccc4)nc(-c4ccccc4)n3)c2)cc1 ; c1ccc(-c2cnc(-c3ccc4oc5ccc6c(c7ccccc7n6-c6ccccc6)c5c4c3)c(-c3nc(-c4ccccc4)nc(-c4ccccc4)n3)c2)cc1]

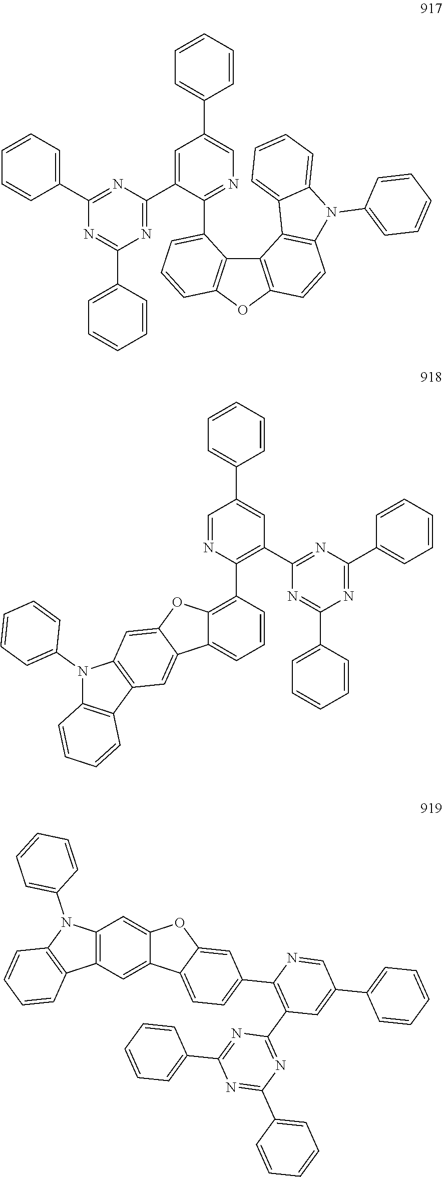 [SMILES: c1ccc(-c2cnc(-c3cccc4oc5ccc6c(c7ccccc7n6-c6ccccc6)c5c34)c(-c3nc(-c4ccccc4)nc(-c4ccccc4)n3)c2)cc1 ; c1ccc(-c2cnc(-c3cccc4c3oc3cc5c(cc34)c3ccccc3n5-c3ccccc3)c(-c3nc(-c4ccccc4)nc(-c4ccccc4)n3)c2)cc1 ; c1ccc(-c2cnc(-c3ccc4c(c3)oc3cc5c(cc34)c3ccccc3n5-c3ccccc3)c(-c3nc(-c4ccccc4)nc(-c4ccccc4)n3)c2)cc1]

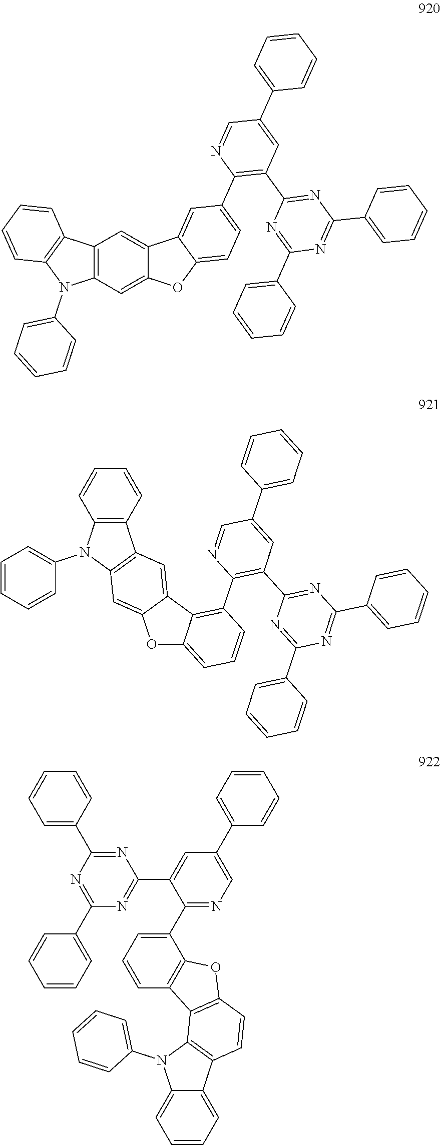

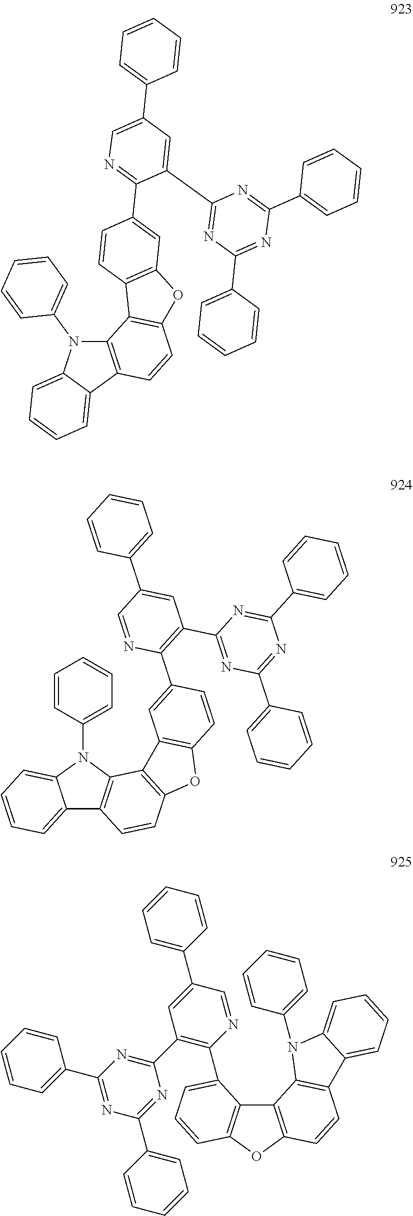 [SMILES: c1ccc(-c2cnc(-c3ccc4c(c3)oc3ccc5c6ccccc6n(-c6ccccc6)c5c34)c(-c3nc(-c4ccccc4)nc(-c4ccccc4)n3)c2)cc1 ; c1ccc(-c2cnc(-c3ccc4oc5ccc6c7ccccc7n(-c7ccccc7)c6c5c4c3)c(-c3nc(-c4ccccc4)nc(-c4ccccc4)n3)c2)cc1 ; c1ccc(-c2cnc(-c3cccc4oc5ccc6c7ccccc7n(-c7ccccc7)c6c5c34)c(-c3nc(-c4ccccc4)nc(-c4ccccc4)n3)c2)cc1]

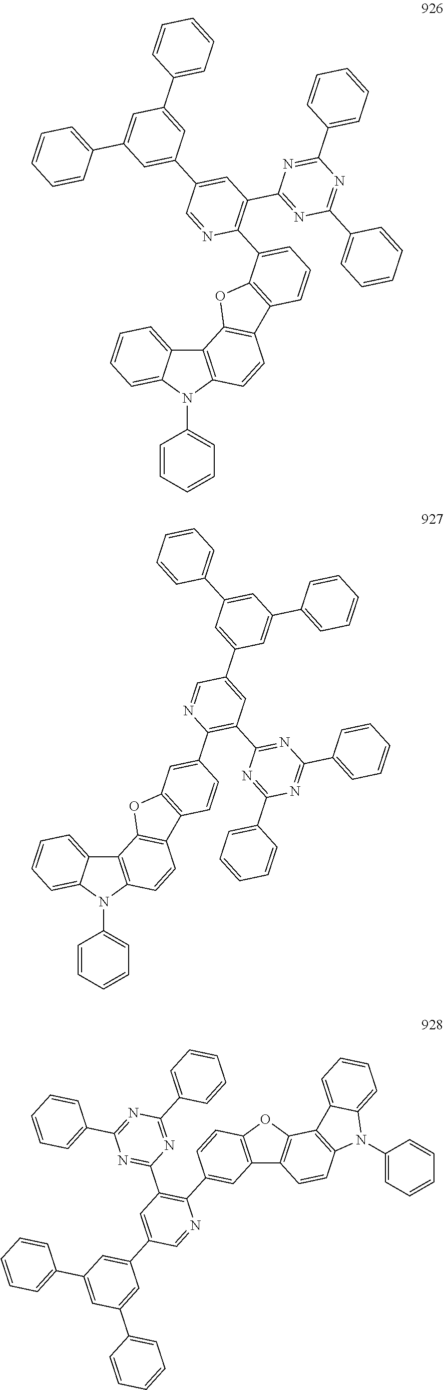

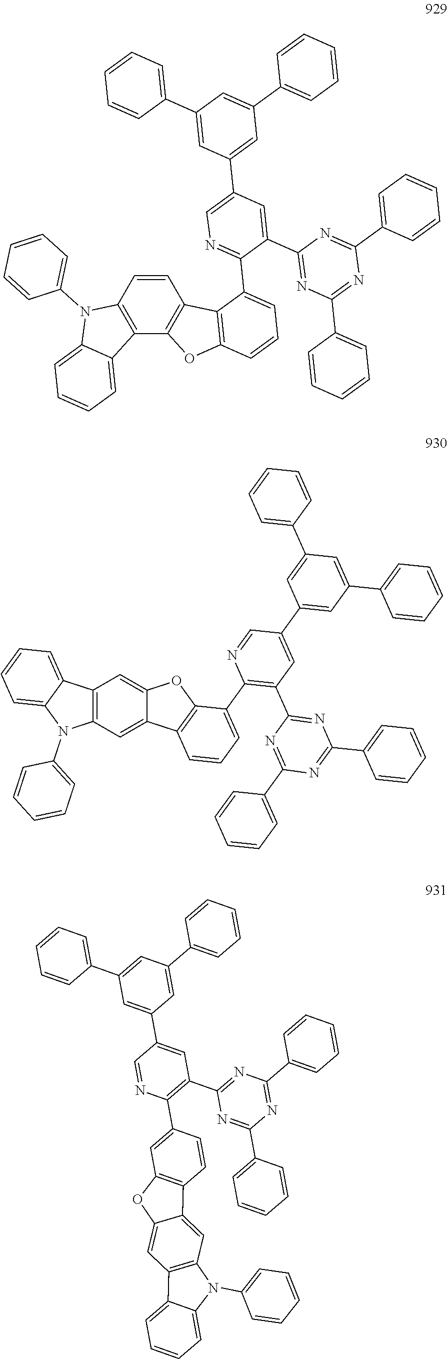 [SMILES: c1ccc(-c2cc(-c3ccccc3)cc(-c3cnc(-c4cccc5oc6c(ccc7c6c6ccccc6n7-c6ccccc6)c45)c(-c4nc(-c5ccccc5)nc(-c5ccccc5)n4)c3)c2)cc1 ; c1ccc(-c2cc(-c3ccccc3)cc(-c3cnc(-c4cccc5c4oc4cc6c7ccccc7n(-c7ccccc7)c6cc45)c(-c4nc(-c5ccccc5)nc(-c5ccccc5)n4)c3)c2)cc1 ; c1ccc(-c2cc(-c3ccccc3)cc(-c3cnc(-c4ccc5c(c4)oc4cc6c7ccccc7n(-c7ccccc7)c6cc45)c(-c4nc(-c5ccccc5)nc(-c5ccccc5)n4)c3)c2)cc1]

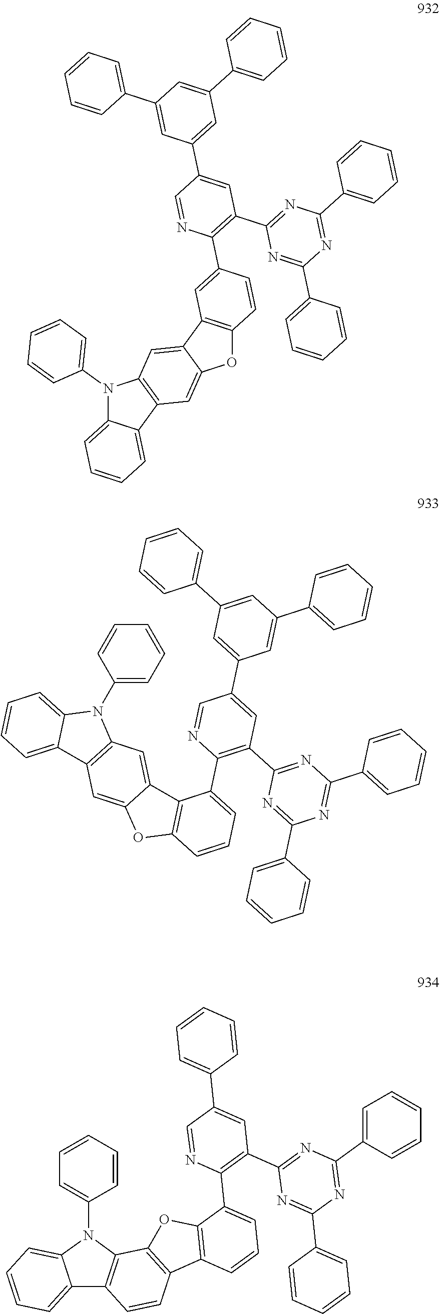 [SMILES: c1ccc(-c2cc(-c3ccccc3)cc(-c3cnc(-c4ccc5oc6cc7c8ccccc8n(-c8ccccc8)c7cc6c5c4)c(-c4nc(-c5ccccc5)nc(-c5ccccc5)n4)c3)c2)cc1 ; c1ccc(-c2cc(-c3ccccc3)cc(-c3cnc(-c4cccc5oc6cc7c8ccccc8n(-c8ccccc8)c7cc6c45)c(-c4nc(-c5ccccc5)nc(-c5ccccc5)n4)c3)c2)cc1 ; c1ccc(-c2cnc(-c3cccc4c3oc3c4ccc4c5ccccc5n(-c5ccccc5)c43)c(-c3nc(-c4ccccc4)nc(-c4ccccc4)n3)c2)cc1]

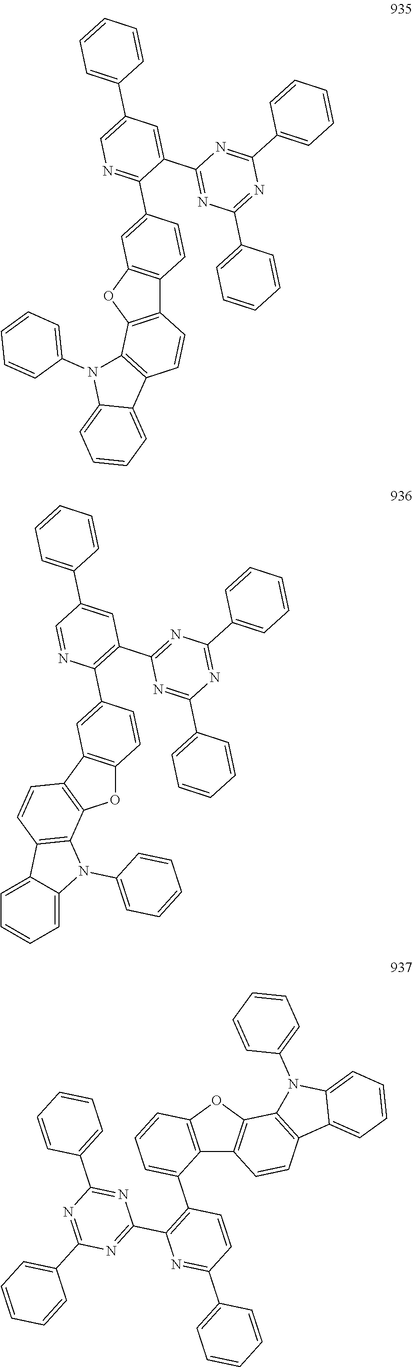 [SMILES: c1ccc(-c2cnc(-c3ccc4c(c3)oc3c4ccc4c5ccccc5n(-c5ccccc5)c43)c(-c3nc(-c4ccccc4)nc(-c4ccccc4)n3)c2)cc1 ; c1ccc(-c2cnc(-c3ccc4oc5c(ccc6c7ccccc7n(-c7ccccc7)c65)c4c3)c(-c3nc(-c4ccccc4)nc(-c4ccccc4)n3)c2)cc1 ; c1ccc(-c2ccc(-c3cccc4oc5c(ccc6c7ccccc7n(-c7ccccc7)c65)c34)c(-c3nc(-c4ccccc4)nc(-c4ccccc4)n3)n2)cc1]

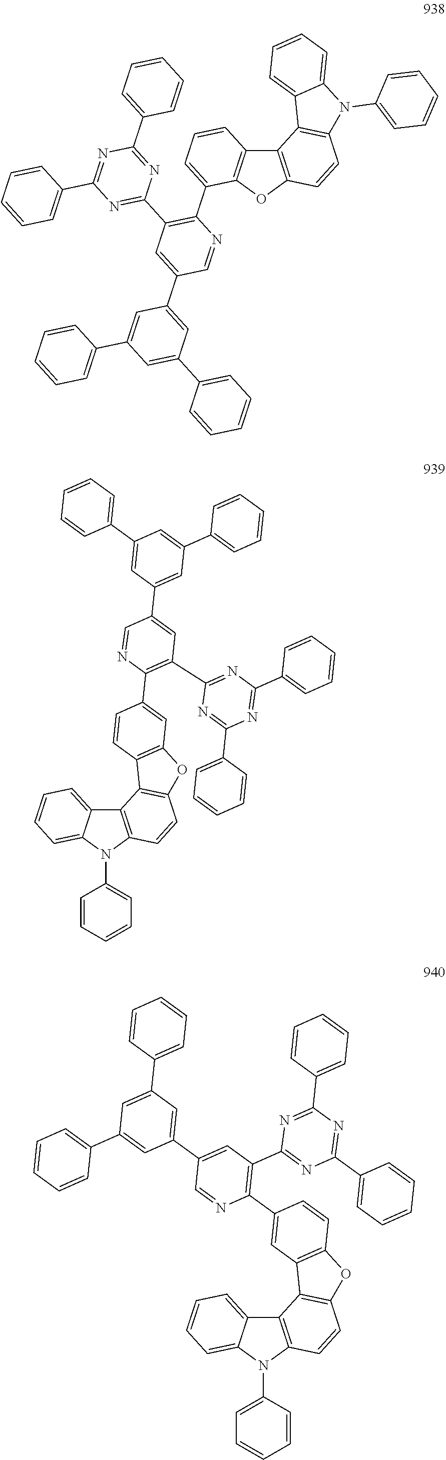

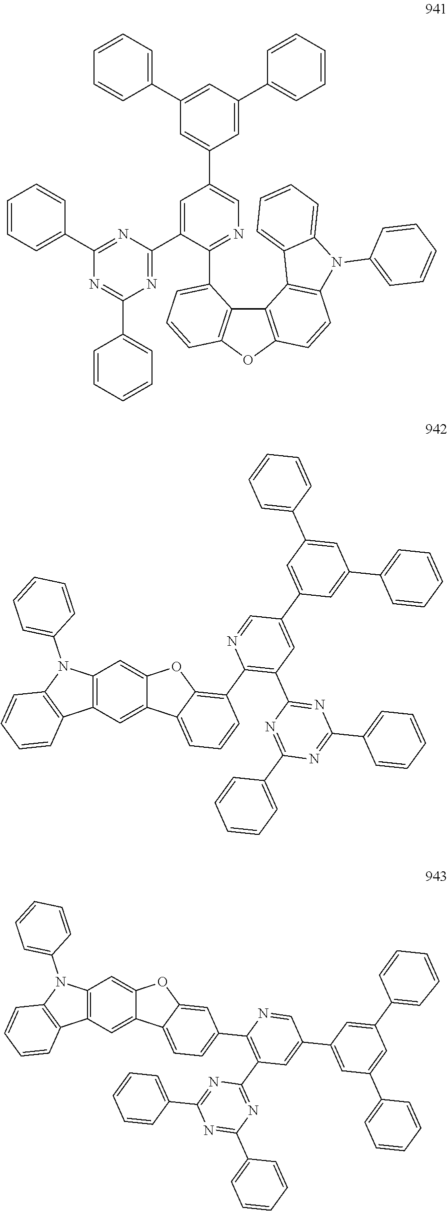 [SMILES: c1ccc(-c2cc(-c3ccccc3)cc(-c3cnc(-c4cccc5oc6ccc7c(c8ccccc8n7-c7ccccc7)c6c45)c(-c4nc(-c5ccccc5)nc(-c5ccccc5)n4)c3)c2)cc1 ; c1ccc(-c2cc(-c3ccccc3)cc(-c3cnc(-c4cccc5c4oc4cc6c(cc45)c4ccccc4n6-c4ccccc4)c(-c4nc(-c5ccccc5)nc(-c5ccccc5)n4)c3)c2)cc1 ; c1ccc(-c2cc(-c3ccccc3)cc(-c3cnc(-c4ccc5c(c4)oc4cc6c(cc45)c4ccccc4n6-c4ccccc4)c(-c4nc(-c5ccccc5)nc(-c5ccccc5)n4)c3)c2)cc1]

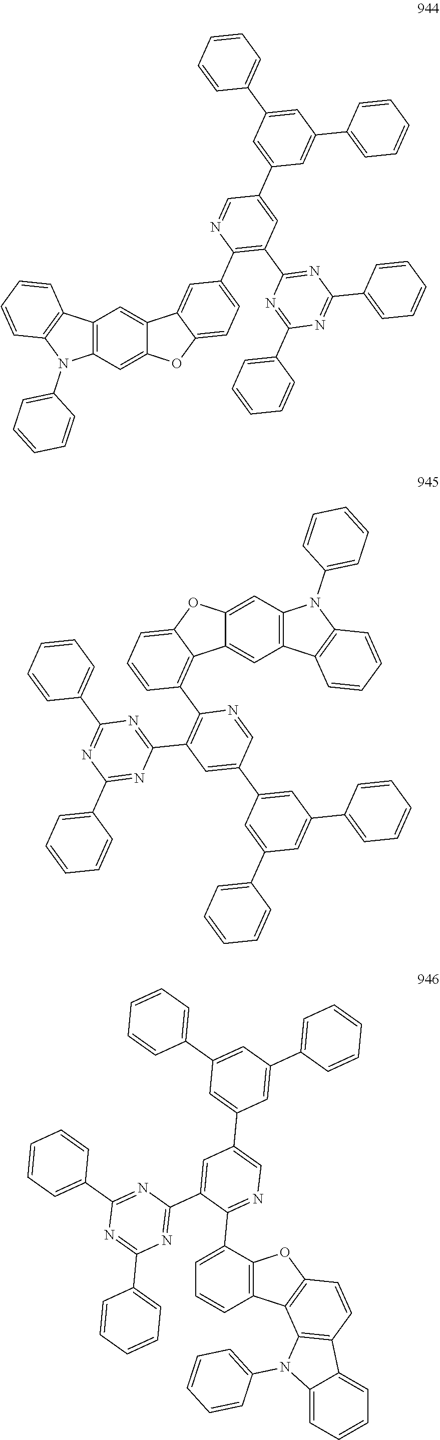 [SMILES: c1ccc(-c2cc(-c3ccccc3)cc(-c3cnc(-c4ccc5oc6cc7c(cc6c5c4)c4ccccc4n7-c4ccccc4)c(-c4nc(-c5ccccc5)nc(-c5ccccc5)n4)c3)c2)cc1 ; c1ccc(-c2cc(-c3ccccc3)cc(-c3cnc(-c4cccc5oc6cc7c(cc6c45)c4ccccc4n7-c4ccccc4)c(-c4nc(-c5ccccc5)nc(-c5ccccc5)n4)c3)c2)cc1 ; c1ccc(-c2cc(-c3ccccc3)cc(-c3cnc(-c4cccc5c4oc4ccc6c7ccccc7n(-c7ccccc7)c6c45)c(-c4nc(-c5ccccc5)nc(-c5ccccc5)n4)c3)c2)cc1]

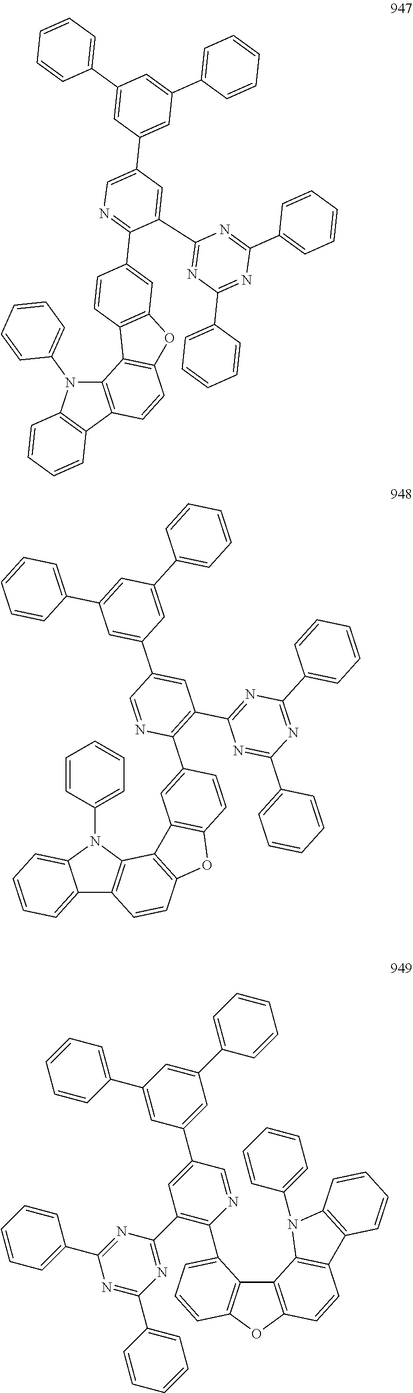 [SMILES: c1ccc(-c2cc(-c3ccccc3)cc(-c3cnc(-c4ccc5c(c4)oc4ccc6c7ccccc7n(-c7ccccc7)c6c45)c(-c4nc(-c5ccccc5)nc(-c5ccccc5)n4)c3)c2)cc1 ; c1ccc(-c2cc(-c3ccccc3)cc(-c3cnc(-c4ccc5oc6ccc7c8ccccc8n(-c8ccccc8)c7c6c5c4)c(-c4nc(-c5ccccc5)nc(-c5ccccc5)n4)c3)c2)cc1 ; c1ccc(-c2cc(-c3ccccc3)cc(-c3cnc(-c4cccc5oc6ccc7c8ccccc8n(-c8ccccc8)c7c6c45)c(-c4nc(-c5ccccc5)nc(-c5ccccc5)n4)c3)c2)cc1]

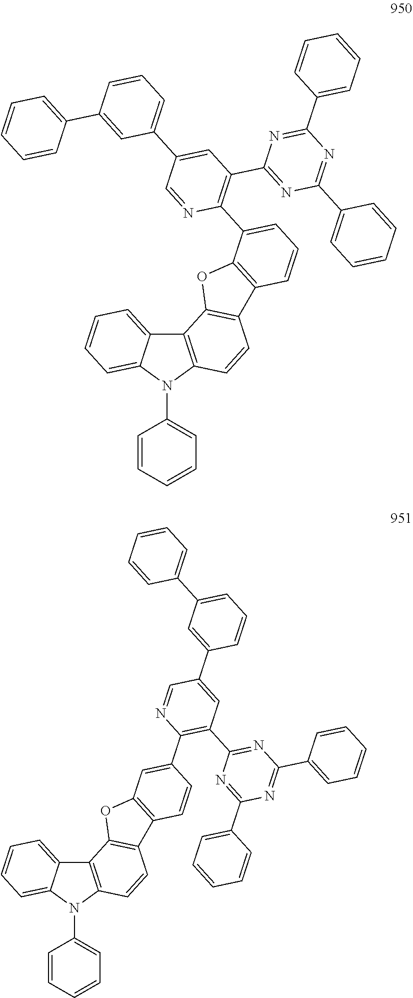

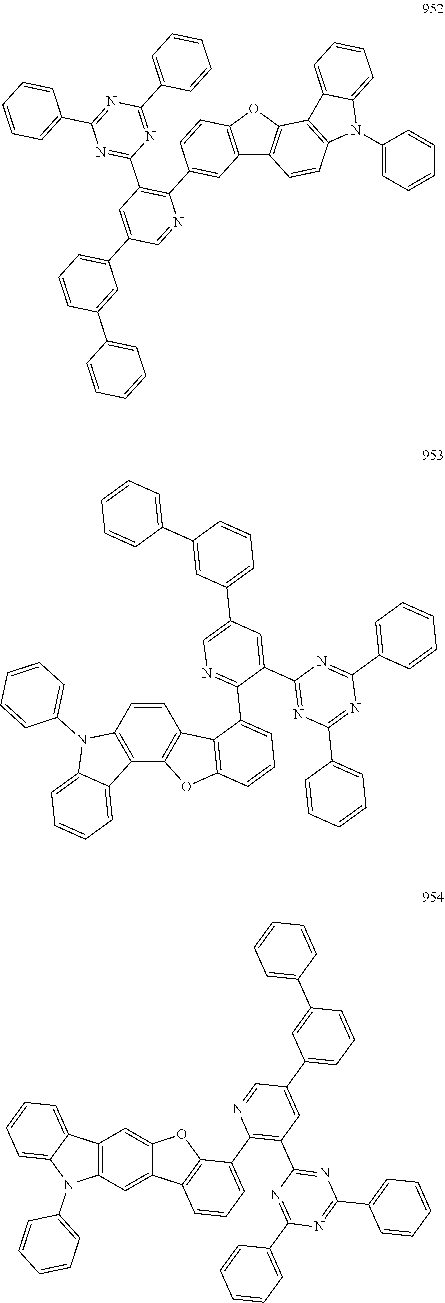 [SMILES: c1ccc(-c2cccc(-c3cnc(-c4ccc5oc6c(ccc7c6c6ccccc6n7-c6ccccc6)c5c4)c(-c4nc(-c5ccccc5)nc(-c5ccccc5)n4)c3)c2)cc1 ; c1ccc(-c2cccc(-c3cnc(-c4cccc5oc6c(ccc7c6c6ccccc6n7-c6ccccc6)c45)c(-c4nc(-c5ccccc5)nc(-c5ccccc5)n4)c3)c2)cc1 ; c1ccc(-c2cccc(-c3cnc(-c4cccc5c4oc4cc6c7ccccc7n(-c7ccccc7)c6cc45)c(-c4nc(-c5ccccc5)nc(-c5ccccc5)n4)c3)c2)cc1]

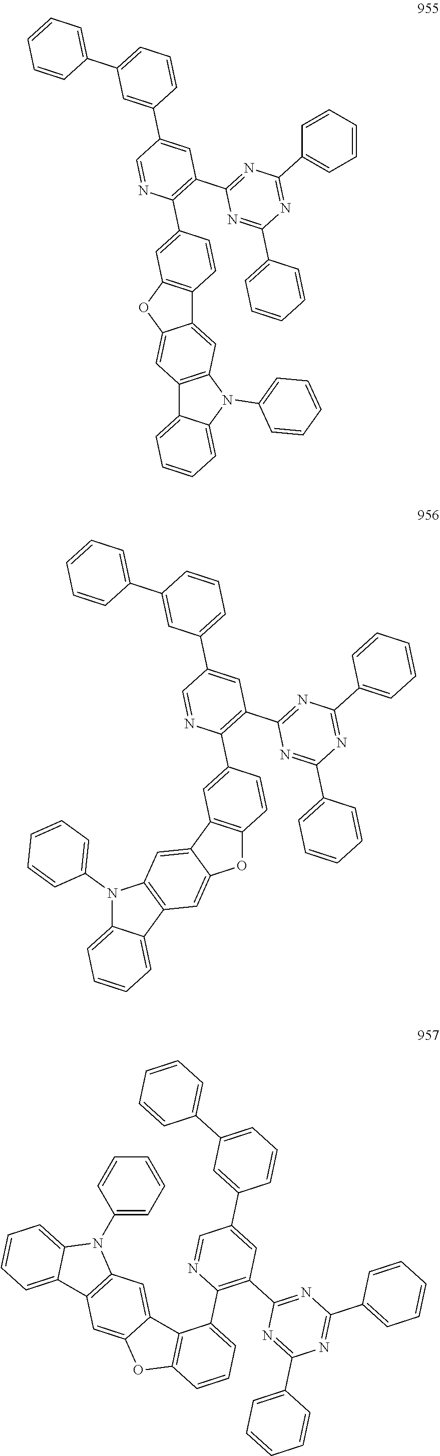 [SMILES: c1ccc(-c2cccc(-c3cnc(-c4ccc5c(c4)oc4cc6c7ccccc7n(-c7ccccc7)c6cc45)c(-c4nc(-c5ccccc5)nc(-c5ccccc5)n4)c3)c2)cc1 ; c1ccc(-c2cccc(-c3cnc(-c4ccc5oc6cc7c8ccccc8n(-c8ccccc8)c7cc6c5c4)c(-c4nc(-c5ccccc5)nc(-c5ccccc5)n4)c3)c2)cc1 ; c1ccc(-c2cccc(-c3cnc(-c4cccc5oc6cc7c8ccccc8n(-c8ccccc8)c7cc6c45)c(-c4nc(-c5ccccc5)nc(-c5ccccc5)n4)c3)c2)cc1]

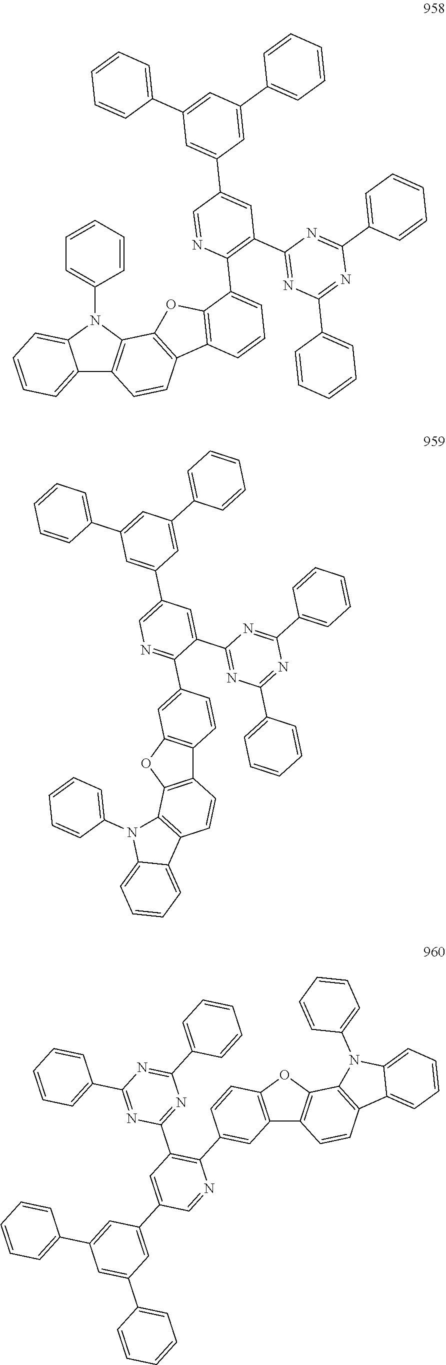 [SMILES: c1ccc(-c2cc(-c3ccccc3)cc(-c3cnc(-c4cccc5c4oc4c5ccc5c6ccccc6n(-c6ccccc6)c54)c(-c4nc(-c5ccccc5)nc(-c5ccccc5)n4)c3)c2)cc1 ; c1ccc(-c2cc(-c3ccccc3)cc(-c3cnc(-c4ccc5c(c4)oc4c5ccc5c6ccccc6n(-c6ccccc6)c54)c(-c4nc(-c5ccccc5)nc(-c5ccccc5)n4)c3)c2)cc1 ; c1ccc(-c2cc(-c3ccccc3)cc(-c3cnc(-c4ccc5oc6c(ccc7c8ccccc8n(-c8ccccc8)c76)c5c4)c(-c4nc(-c5ccccc5)nc(-c5ccccc5)n4)c3)c2)cc1]

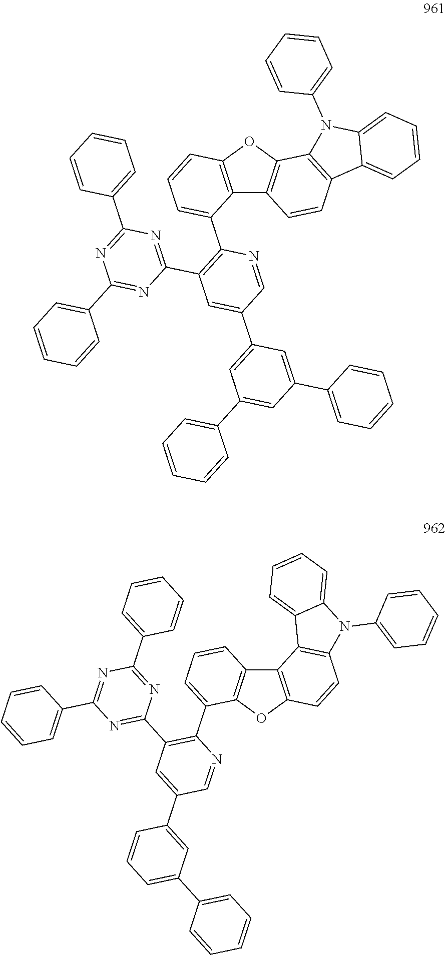 [SMILES: c1ccc(-c2cc(-c3ccccc3)cc(-c3cnc(-c4cccc5oc6c(ccc7c8ccccc8n(-c8ccccc8)c76)c45)c(-c4nc(-c5ccccc5)nc(-c5ccccc5)n4)c3)c2)cc1 ; c1ccc(-c2cccc(-c3cnc(-c4cccc5c4oc4ccc6c(c7ccccc7n6-c6ccccc6)c45)c(-c4nc(-c5ccccc5)nc(-c5ccccc5)n4)c3)c2)cc1]

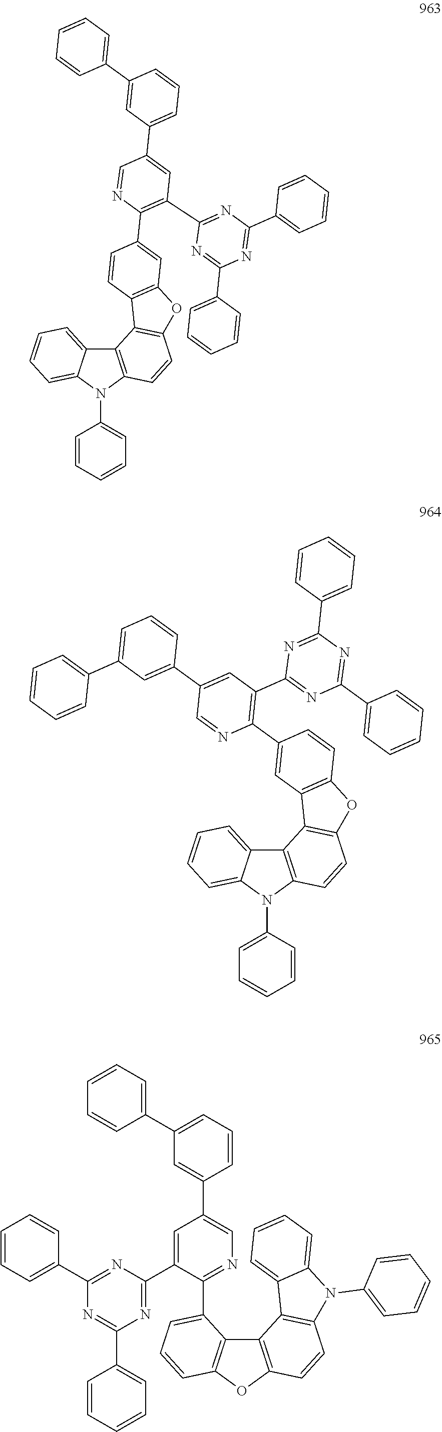 [SMILES: c1ccc(-c2cccc(-c3cnc(-c4ccc5c(c4)oc4ccc6c(c7ccccc7n6-c6ccccc6)c45)c(-c4nc(-c5ccccc5)nc(-c5ccccc5)n4)c3)c2)cc1 ; c1ccc(-c2cccc(-c3cnc(-c4ccc5oc6ccc7c(c8ccccc8n7-c7ccccc7)c6c5c4)c(-c4nc(-c5ccccc5)nc(-c5ccccc5)n4)c3)c2)cc1 ; c1ccc(-c2cccc(-c3cnc(-c4cccc5oc6ccc7c(c8ccccc8n7-c7ccccc7)c6c45)c(-c4nc(-c5ccccc5)nc(-c5ccccc5)n4)c3)c2)cc1]

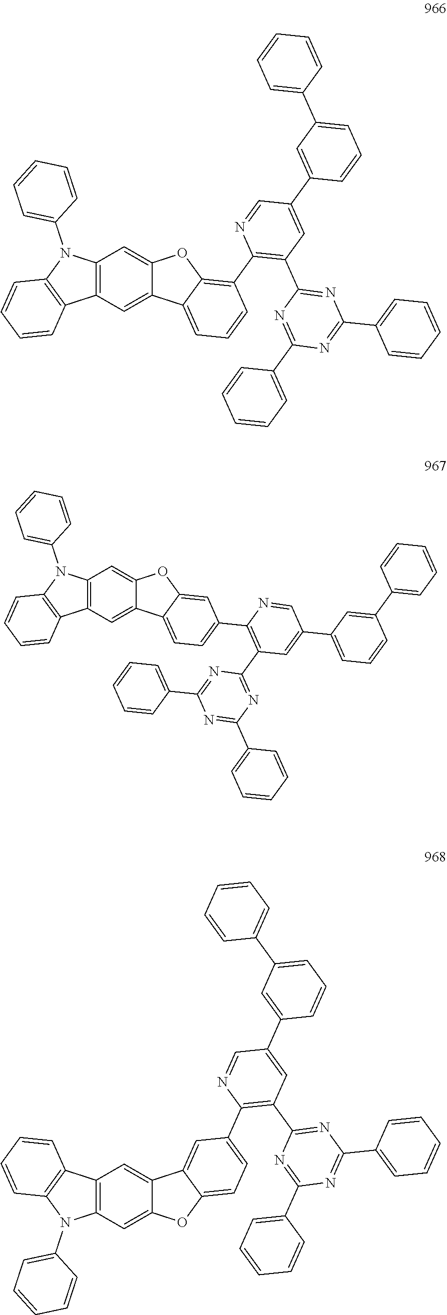 [SMILES: c1ccc(-c2cccc(-c3cnc(-c4cccc5c4oc4cc6c(cc45)c4ccccc4n6-c4ccccc4)c(-c4nc(-c5ccccc5)nc(-c5ccccc5)n4)c3)c2)cc1 ; c1ccc(-c2cccc(-c3cnc(-c4ccc5c(c4)oc4cc6c(cc45)c4ccccc4n6-c4ccccc4)c(-c4nc(-c5ccccc5)nc(-c5ccccc5)n4)c3)c2)cc1 ; c1ccc(-c2cccc(-c3cnc(-c4ccc5oc6cc7c(cc6c5c4)c4ccccc4n7-c4ccccc4)c(-c4nc(-c5ccccc5)nc(-c5ccccc5)n4)c3)c2)cc1]

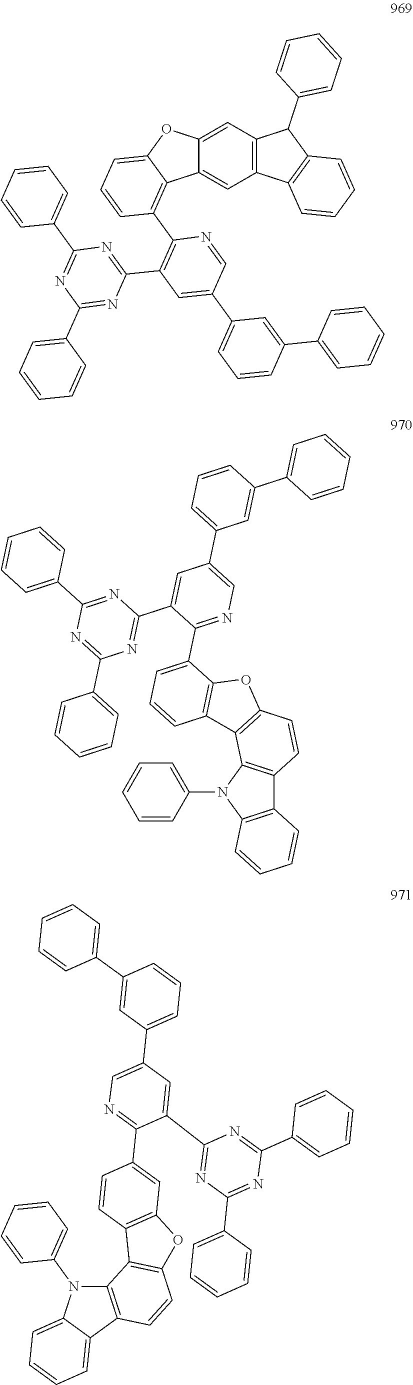 [SMILES: c1ccc(-c2cccc(-c3cnc(-c4cccc5oc6cc7c(cc6c45)-c4ccccc4C7c4ccccc4)c(-c4nc(-c5ccccc5)nc(-c5ccccc5)n4)c3)c2)cc1 ; c1ccc(-c2cccc(-c3cnc(-c4cccc5c4oc4ccc6c7ccccc7n(-c7ccccc7)c6c45)c(-c4nc(-c5ccccc5)nc(-c5ccccc5)n4)c3)c2)cc1 ; c1ccc(-c2cccc(-c3cnc(-c4ccc5c(c4)oc4ccc6c7ccccc7n(-c7ccccc7)c6c45)c(-c4nc(-c5ccccc5)nc(-c5ccccc5)n4)c3)c2)cc1]

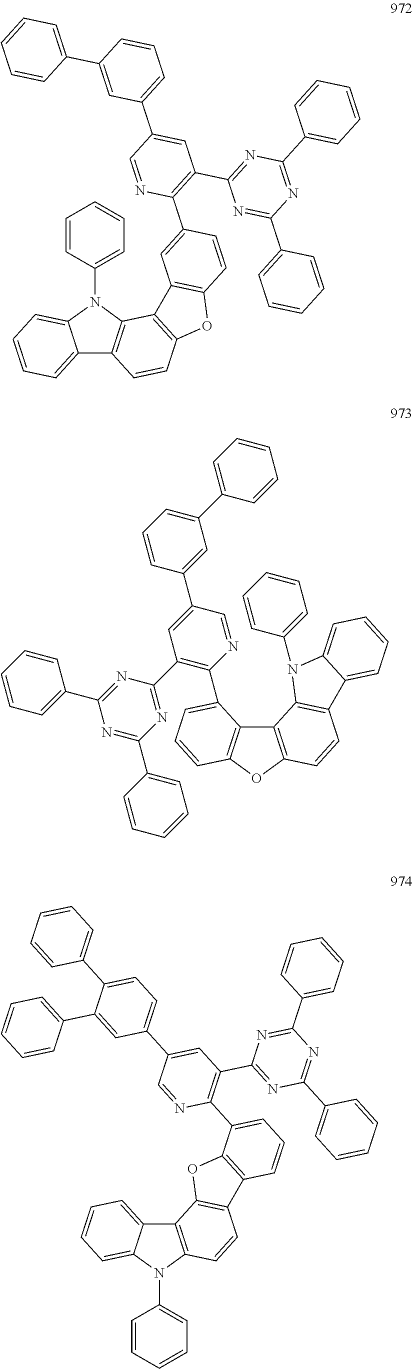 [SMILES: c1ccc(-c2cccc(-c3cnc(-c4ccc5oc6ccc7c8ccccc8n(-c8ccccc8)c7c6c5c4)c(-c4nc(-c5ccccc5)nc(-c5ccccc5)n4)c3)c2)cc1 ; c1ccc(-c2cccc(-c3cnc(-c4cccc5oc6ccc7c8ccccc8n(-c8ccccc8)c7c6c45)c(-c4nc(-c5ccccc5)nc(-c5ccccc5)n4)c3)c2)cc1 ; c1ccc(-c2nc(-c3ccccc3)nc(-c3cc(-c4ccc(-c5ccccc5)c(-c5ccccc5)c4)cnc3-c3cccc4c3oc3c4ccc4c3c3ccccc3n4-c3ccccc3)n2)cc1]

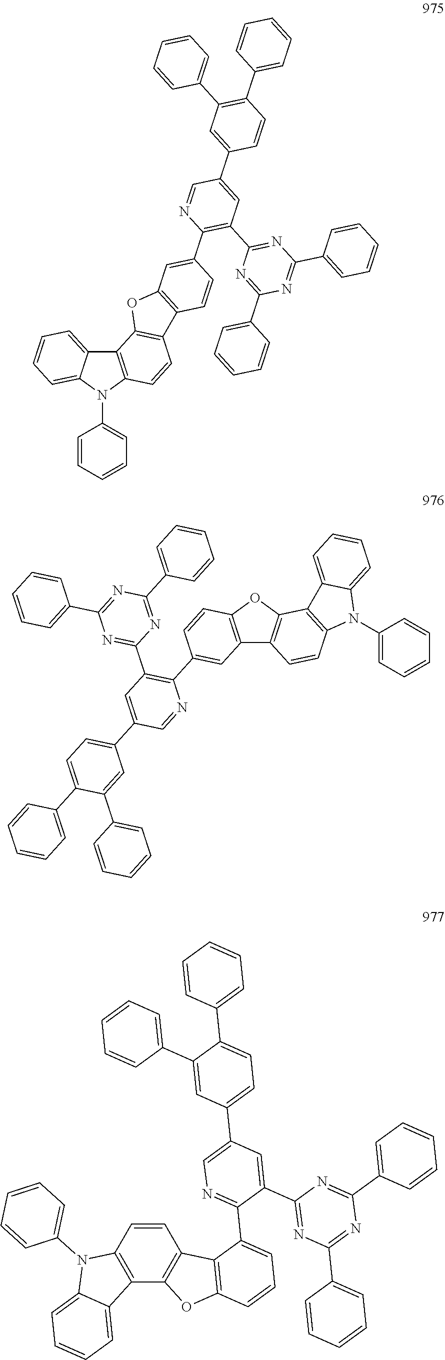 [SMILES: c1ccc(-c2nc(-c3ccccc3)nc(-c3cc(-c4ccc(-c5ccccc5)c(-c5ccccc5)c4)cnc3-c3ccc4c(c3)oc3c4ccc4c3c3ccccc3n4-c3ccccc3)n2)cc1 ; c1ccc(-c2nc(-c3ccccc3)nc(-c3cc(-c4ccc(-c5ccccc5)c(-c5ccccc5)c4)cnc3-c3ccc4oc5c(ccc6c5c5ccccc5n6-c5ccccc5)c4c3)n2)cc1 ; c1ccc(-c2nc(-c3ccccc3)nc(-c3cc(-c4ccc(-c5ccccc5)c(-c5ccccc5)c4)cnc3-c3cccc4oc5c(ccc6c5c5ccccc5n6-c5ccccc5)c34)n2)cc1]

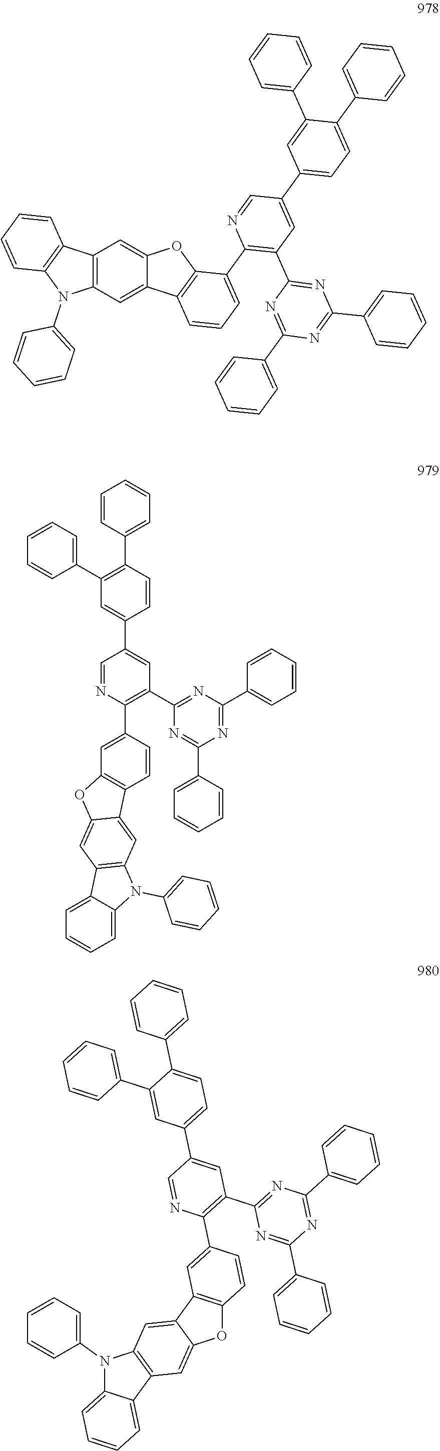 [SMILES: c1ccc(-c2nc(-c3ccccc3)nc(-c3cc(-c4ccc(-c5ccccc5)c(-c5ccccc5)c4)cnc3-c3cccc4c3oc3cc5c6ccccc6n(-c6ccccc6)c5cc34)n2)cc1 ; c1ccc(-c2nc(-c3ccccc3)nc(-c3cc(-c4ccc(-c5ccccc5)c(-c5ccccc5)c4)cnc3-c3ccc4c(c3)oc3cc5c6ccccc6n(-c6ccccc6)c5cc34)n2)cc1 ; c1ccc(-c2nc(-c3ccccc3)nc(-c3cc(-c4ccc(-c5ccccc5)c(-c5ccccc5)c4)cnc3-c3ccc4oc5cc6c7ccccc7n(-c7ccccc7)c6cc5c4c3)n2)cc1]

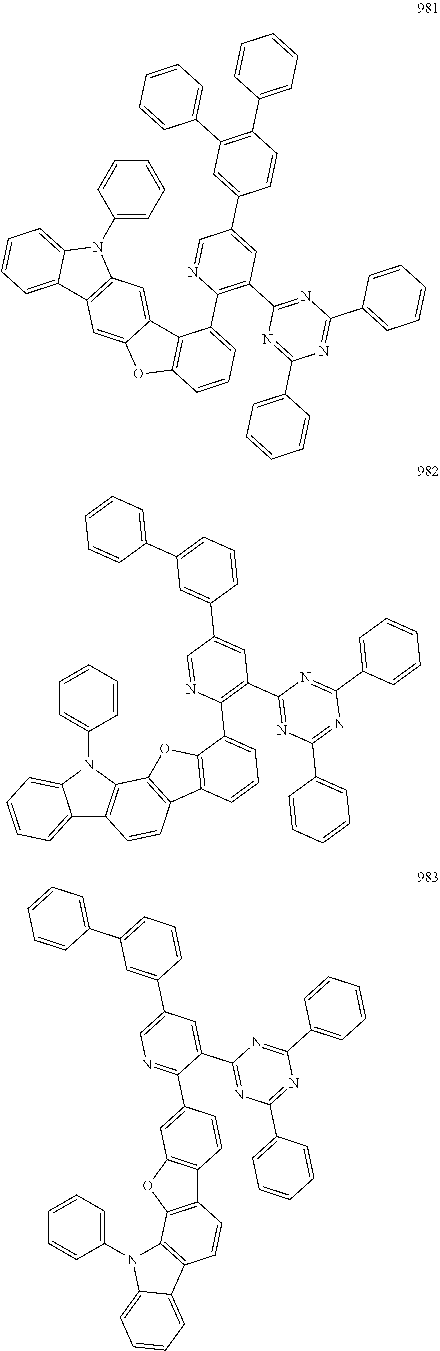

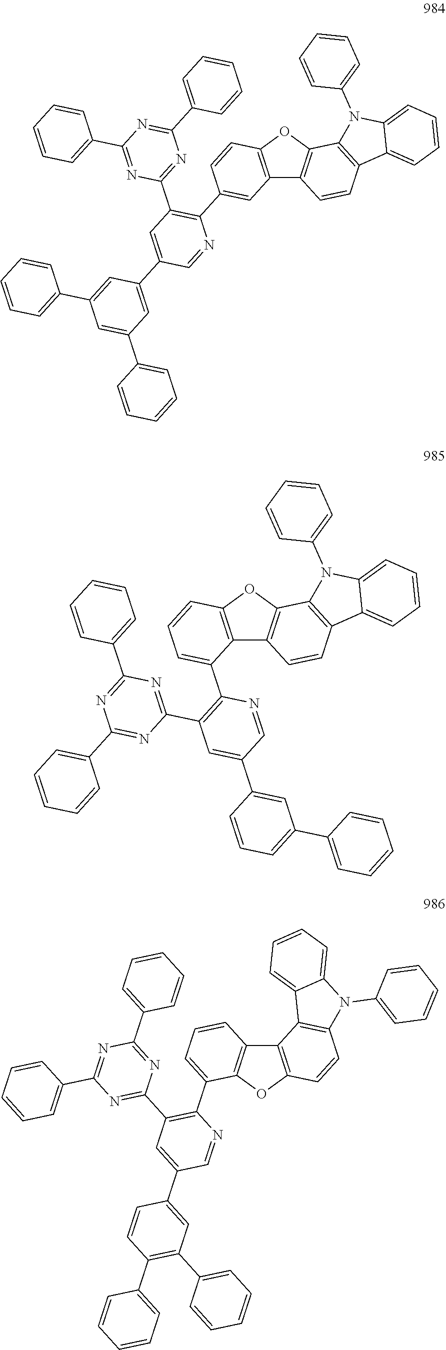 [SMILES: c1ccc(-c2cc(-c3ccccc3)cc(-c3cnc(-c4ccc5oc6c(ccc7c8ccccc8n(-c8ccccc8)c76)c5c4)c(-c4nc(-c5ccccc5)nc(-c5ccccc5)n4)c3)c2)cc1 ; c1ccc(-c2cccc(-c3cnc(-c4cccc5oc6c(ccc7c8ccccc8n(-c8ccccc8)c76)c45)c(-c4nc(-c5ccccc5)nc(-c5ccccc5)n4)c3)c2)cc1 ; c1ccc(-c2nc(-c3ccccc3)nc(-c3cc(-c4ccc(-c5ccccc5)c(-c5ccccc5)c4)cnc3-c3cccc4c3oc3ccc5c(c6ccccc6n5-c5ccccc5)c34)n2)cc1]

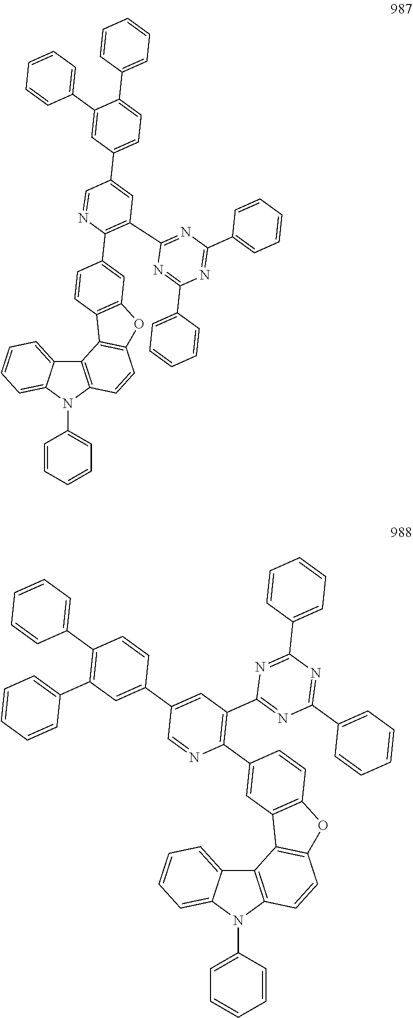 [SMILES: c1ccc(-c2nc(-c3ccccc3)nc(-c3cc(-c4ccc(-c5ccccc5)c(-c5ccccc5)c4)cnc3-c3ccc4c(c3)oc3ccc5c(c6ccccc6n5-c5ccccc5)c34)n2)cc1 ; c1ccc(-c2nc(-c3ccccc3)nc(-c3cc(-c4ccc(-c5ccccc5)c(-c5ccccc5)c4)cnc3-c3ccc4oc5ccc6c(c7ccccc7n6-c6ccccc6)c5c4c3)n2)cc1]

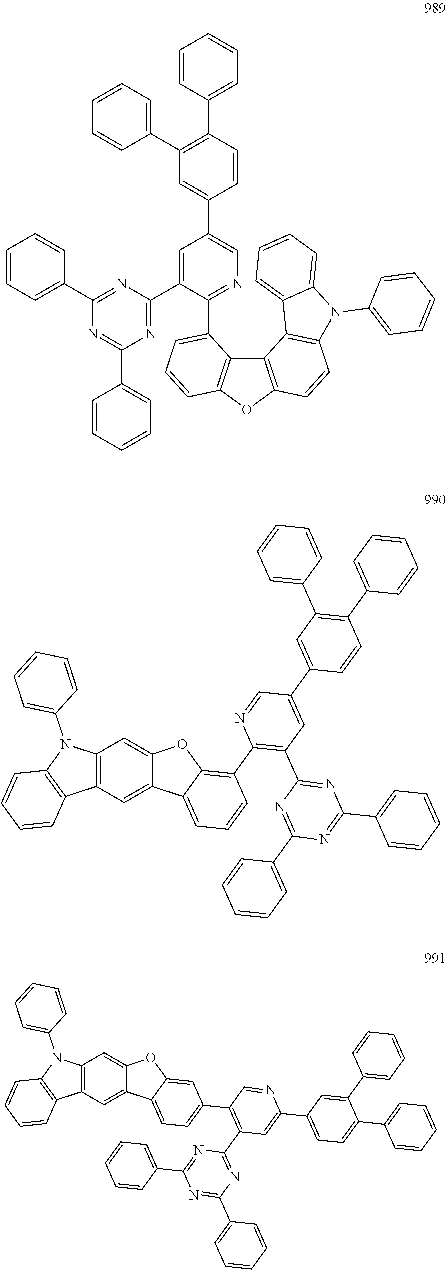 [SMILES: c1ccc(-c2nc(-c3ccccc3)nc(-c3cc(-c4ccc(-c5ccccc5)c(-c5ccccc5)c4)cnc3-c3cccc4oc5ccc6c(c7ccccc7n6-c6ccccc6)c5c34)n2)cc1 ; c1ccc(-c2nc(-c3ccccc3)nc(-c3cc(-c4ccc(-c5ccccc5)c(-c5ccccc5)c4)cnc3-c3cccc4c3oc3cc5c(cc34)c3ccccc3n5-c3ccccc3)n2)cc1 ; c1ccc(-c2nc(-c3ccccc3)nc(-c3cc(-c4ccc(-c5ccccc5)c(-c5ccccc5)c4)ncc3-c3ccc4c(c3)oc3cc5c(cc34)c3ccccc3n5-c3ccccc3)n2)cc1]

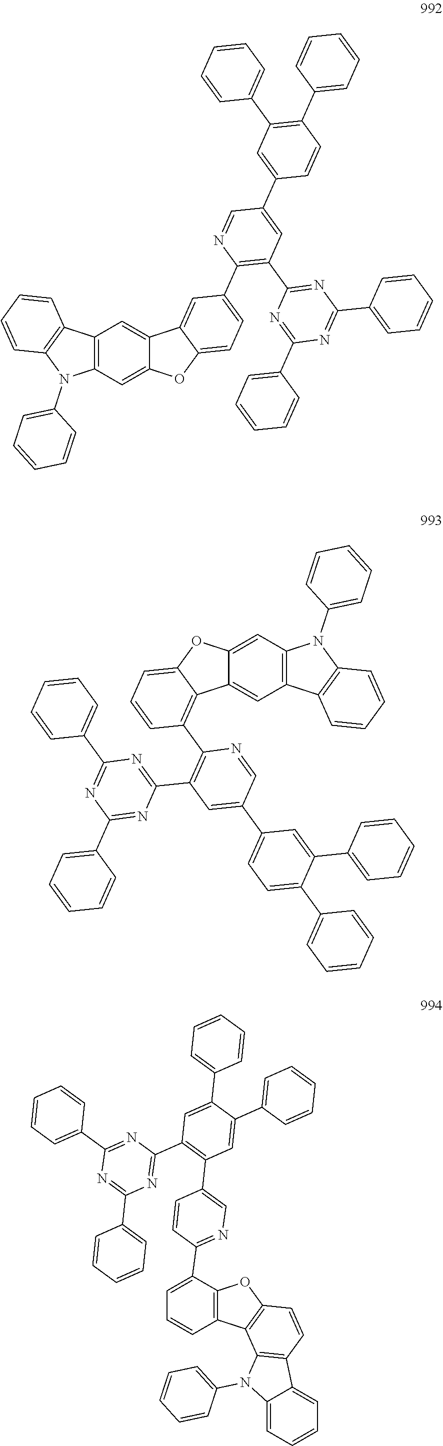 [SMILES: c1ccc(-c2nc(-c3ccccc3)nc(-c3cc(-c4ccc(-c5ccccc5)c(-c5ccccc5)c4)cnc3-c3ccc4oc5cc6c(cc5c4c3)c3ccccc3n6-c3ccccc3)n2)cc1 ; c1ccc(-c2nc(-c3ccccc3)nc(-c3cc(-c4ccc(-c5ccccc5)c(-c5ccccc5)c4)cnc3-c3cccc4oc5cc6c(cc5c34)c3ccccc3n6-c3ccccc3)n2)cc1 ; c1ccc(-c2nc(-c3ccccc3)nc(-c3cc(-c4ccccc4)c(-c4ccccc4)cc3-c3ccc(-c4cccc5c4oc4ccc6c7ccccc7n(-c7ccccc7)c6c45)nc3)n2)cc1]

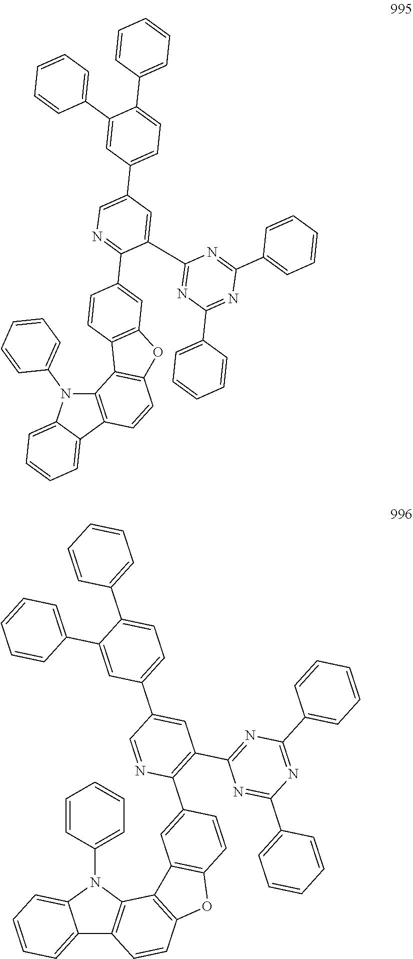 [SMILES: c1ccc(-c2nc(-c3ccccc3)nc(-c3cc(-c4ccc(-c5ccccc5)c(-c5ccccc5)c4)cnc3-c3ccc4c(c3)oc3ccc5c6ccccc6n(-c6ccccc6)c5c34)n2)cc1 ; c1ccc(-c2nc(-c3ccccc3)nc(-c3cc(-c4ccc(-c5ccccc5)c(-c5ccccc5)c4)cnc3-c3ccc4oc5ccc6c7ccccc7n(-c7ccccc7)c6c5c4c3)n2)cc1]

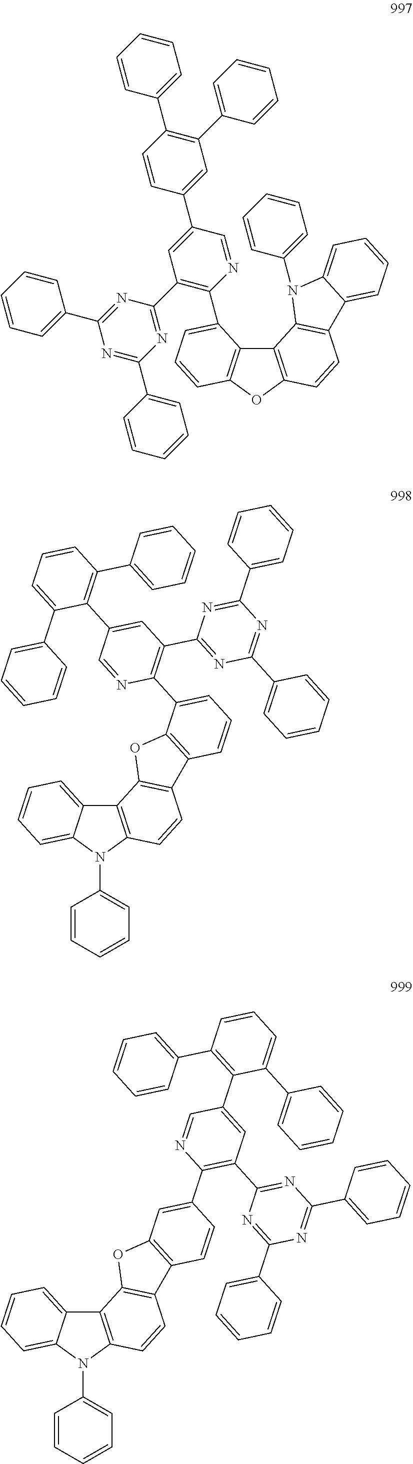 [SMILES: c1ccc(-c2nc(-c3ccccc3)nc(-c3cc(-c4ccc(-c5ccccc5)c(-c5ccccc5)c4)cnc3-c3cccc4oc5ccc6c7ccccc7n(-c7ccccc7)c6c5c34)n2)cc1 ; c1ccc(-c2nc(-c3ccccc3)nc(-c3cc(-c4c(-c5ccccc5)cccc4-c4ccccc4)cnc3-c3cccc4c3oc3c4ccc4c3c3ccccc3n4-c3ccccc3)n2)cc1 ; c1ccc(-c2nc(-c3ccccc3)nc(-c3cc(-c4c(-c5ccccc5)cccc4-c4ccccc4)cnc3-c3ccc4c(c3)oc3c4ccc4c3c3ccccc3n4-c3ccccc3)n2)cc1]

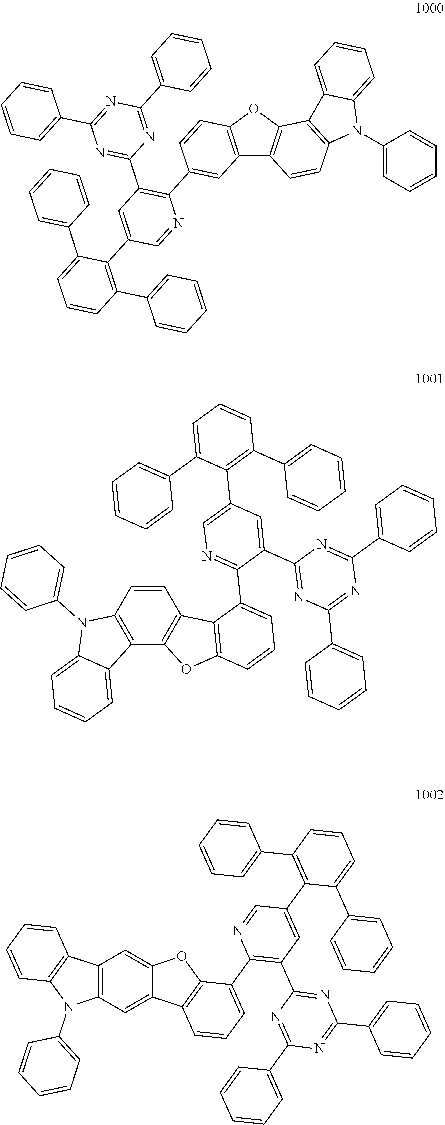 [SMILES: c1ccc(-c2nc(-c3ccccc3)nc(-c3cc(-c4c(-c5ccccc5)cccc4-c4ccccc4)cnc3-c3ccc4oc5c(ccc6c5c5ccccc5n6-c5ccccc5)c4c3)n2)cc1 ; c1ccc(-c2nc(-c3ccccc3)nc(-c3cc(-c4c(-c5ccccc5)cccc4-c4ccccc4)cnc3-c3cccc4oc5c(ccc6c5c5ccccc5n6-c5ccccc5)c34)n2)cc1 ; c1ccc(-c2nc(-c3ccccc3)nc(-c3cc(-c4c(-c5ccccc5)cccc4-c4ccccc4)cnc3-c3cccc4c3oc3cc5c6ccccc6n(-c6ccccc6)c5cc34)n2)cc1]

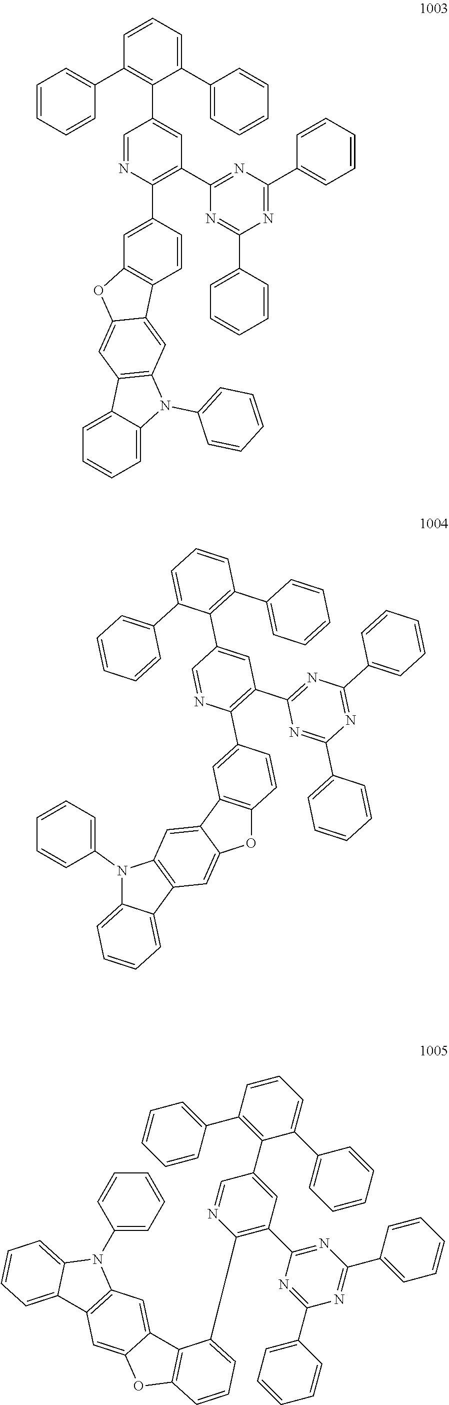 [SMILES: c1ccc(-c2nc(-c3ccccc3)nc(-c3cc(-c4c(-c5ccccc5)cccc4-c4ccccc4)cnc3-c3ccc4c(c3)oc3cc5c6ccccc6n(-c6ccccc6)c5cc34)n2)cc1 ; c1ccc(-c2nc(-c3ccccc3)nc(-c3cc(-c4c(-c5ccccc5)cccc4-c4ccccc4)cnc3-c3ccc4oc5cc6c7ccccc7n(-c7ccccc7)c6cc5c4c3)n2)cc1 ; c1ccc(-c2nc(-c3ccccc3)nc(-c3cc(-c4c(-c5ccccc5)cccc4-c4ccccc4)cnc3-c3cccc4oc5cc6c7ccccc7n(-c7ccccc7)c6cc5c34)n2)cc1]

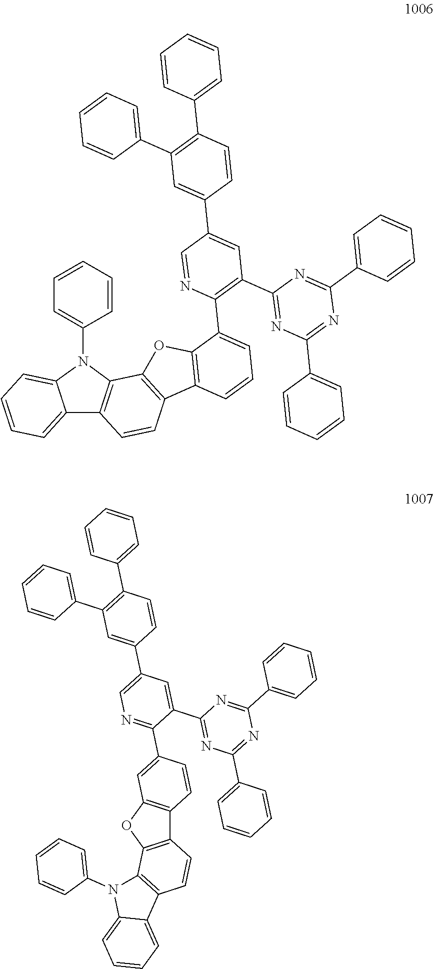 [SMILES: c1ccc(-c2nc(-c3ccccc3)nc(-c3cc(-c4ccc(-c5ccccc5)c(-c5ccccc5)c4)cnc3-c3cccc4c3oc3c4ccc4c5ccccc5n(-c5ccccc5)c43)n2)cc1 ; c1ccc(-c2nc(-c3ccccc3)nc(-c3cc(-c4ccc(-c5ccccc5)c(-c5ccccc5)c4)cnc3-c3ccc4c(c3)oc3c4ccc4c5ccccc5n(-c5ccccc5)c43)n2)cc1]

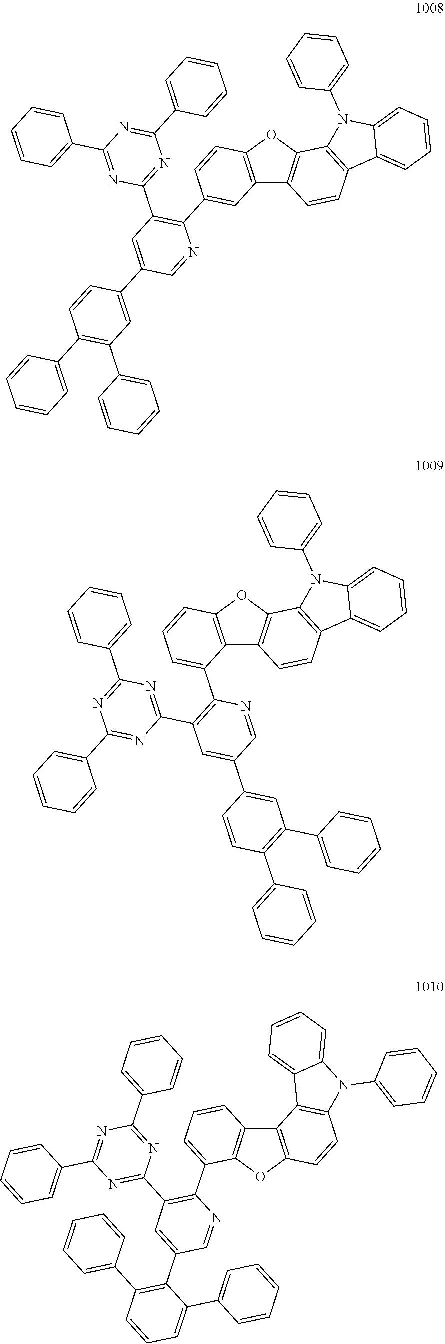 [SMILES: c1ccc(-c2nc(-c3ccccc3)nc(-c3cc(-c4ccc(-c5ccccc5)c(-c5ccccc5)c4)cnc3-c3ccc4oc5c(ccc6c7ccccc7n(-c7ccccc7)c65)c4c3)n2)cc1 ; c1ccc(-c2nc(-c3ccccc3)nc(-c3cc(-c4ccc(-c5ccccc5)c(-c5ccccc5)c4)cnc3-c3cccc4oc5c(ccc6c7ccccc7n(-c7ccccc7)c65)c34)n2)cc1 ; c1ccc(-c2nc(-c3ccccc3)nc(-c3cc(-c4c(-c5ccccc5)cccc4-c4ccccc4)cnc3-c3cccc4c3oc3ccc5c(c6ccccc6n5-c5ccccc5)c34)n2)cc1]

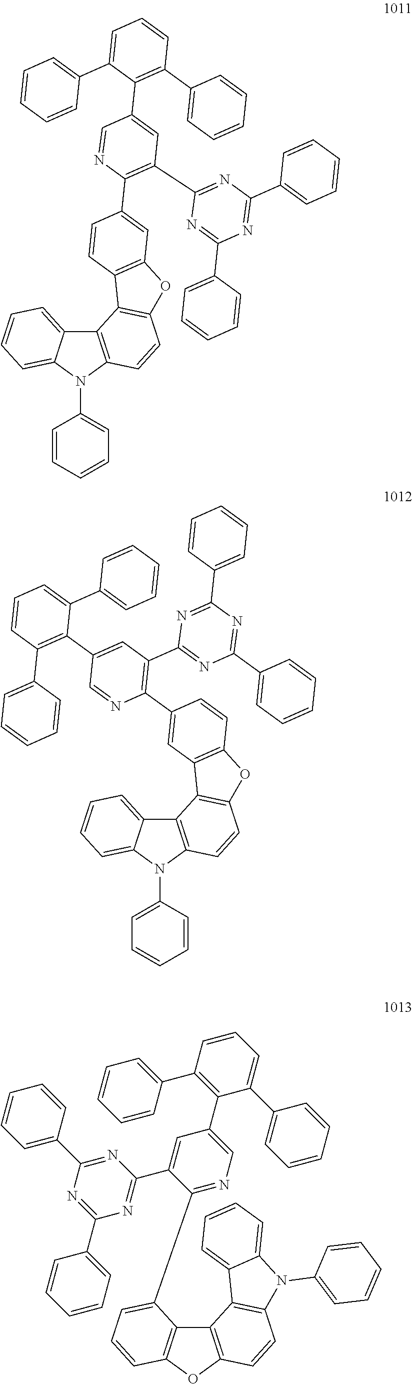 [SMILES: c1ccc(-c2nc(-c3ccccc3)nc(-c3cc(-c4c(-c5ccccc5)cccc4-c4ccccc4)cnc3-c3ccc4c(c3)oc3ccc5c(c6ccccc6n5-c5ccccc5)c34)n2)cc1 ; c1ccc(-c2nc(-c3ccccc3)nc(-c3cc(-c4c(-c5ccccc5)cccc4-c4ccccc4)cnc3-c3ccc4oc5ccc6c(c7ccccc7n6-c6ccccc6)c5c4c3)n2)cc1 ; c1ccc(-c2nc(-c3ccccc3)nc(-c3cc(-c4c(-c5ccccc5)cccc4-c4ccccc4)cnc3-c3cccc4oc5ccc6c(c7ccccc7n6-c6ccccc6)c5c34)n2)cc1]

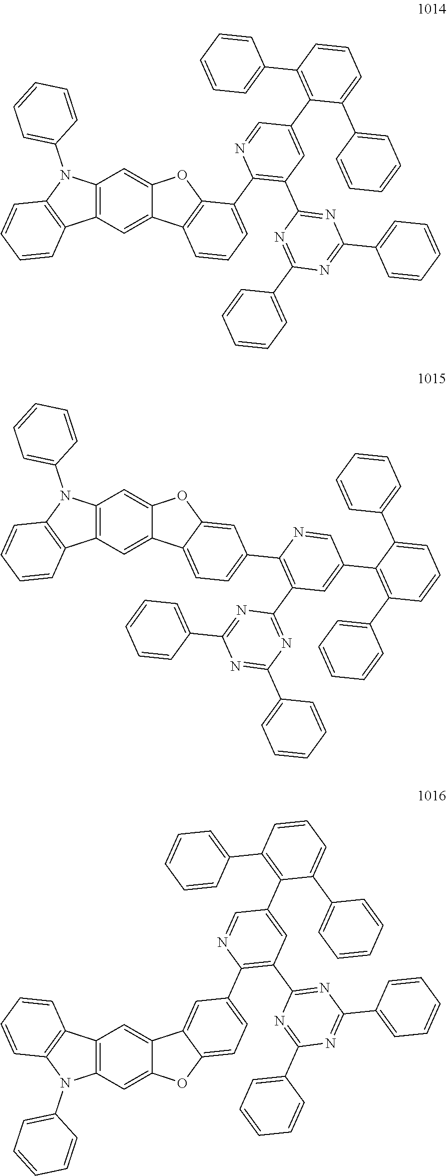 [SMILES: c1ccc(-c2nc(-c3ccccc3)nc(-c3cc(-c4c(-c5ccccc5)cccc4-c4ccccc4)cnc3-c3cccc4c3oc3cc5c(cc34)c3ccccc3n5-c3ccccc3)n2)cc1 ; c1ccc(-c2nc(-c3ccccc3)nc(-c3cc(-c4c(-c5ccccc5)cccc4-c4ccccc4)cnc3-c3ccc4c(c3)oc3cc5c(cc34)c3ccccc3n5-c3ccccc3)n2)cc1 ; c1ccc(-c2nc(-c3ccccc3)nc(-c3cc(-c4c(-c5ccccc5)cccc4-c4ccccc4)cnc3-c3ccc4oc5cc6c(cc5c4c3)c3ccccc3n6-c3ccccc3)n2)cc1]

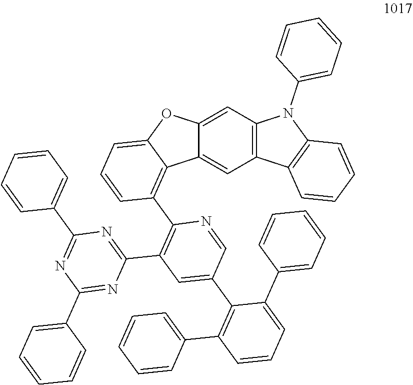 [SMILES: c1ccc(-c2nc(-c3ccccc3)nc(-c3cc(-c4c(-c5ccccc5)cccc4-c4ccccc4)cnc3-c3cccc4oc5cc6c(cc5c34)c3ccccc3n6-c3ccccc3)n2)cc1]

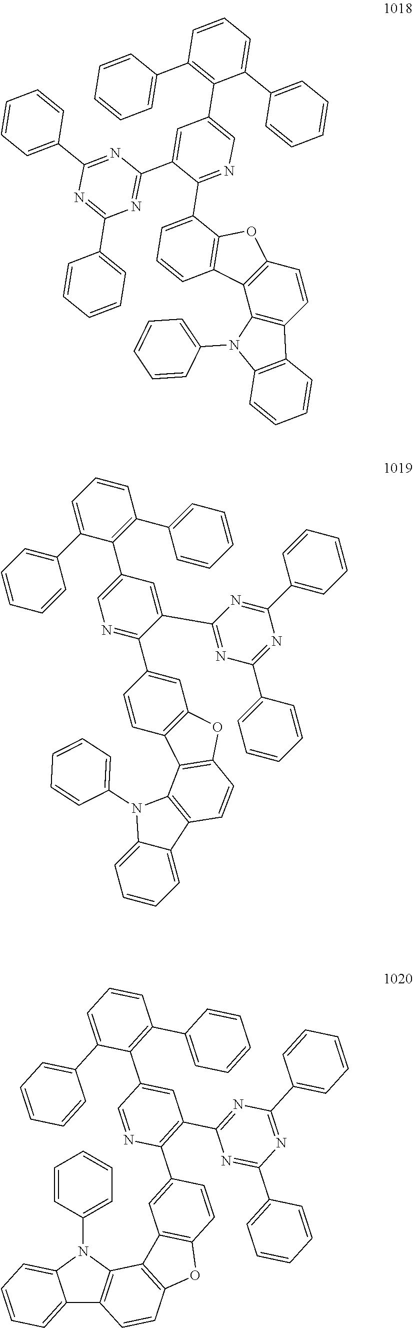 [SMILES: c1ccc(-c2nc(-c3ccccc3)nc(-c3cc(-c4c(-c5ccccc5)cccc4-c4ccccc4)cnc3-c3cccc4c3oc3ccc5c6ccccc6n(-c6ccccc6)c5c34)n2)cc1 ; c1ccc(-c2nc(-c3ccccc3)nc(-c3cc(-c4c(-c5ccccc5)cccc4-c4ccccc4)cnc3-c3ccc4c(c3)oc3ccc5c6ccccc6n(-c6ccccc6)c5c34)n2)cc1 ; c1ccc(-c2nc(-c3ccccc3)nc(-c3cc(-c4c(-c5ccccc5)cccc4-c4ccccc4)cnc3-c3ccc4oc5ccc6c7ccccc7n(-c7ccccc7)c6c5c4c3)n2)cc1]

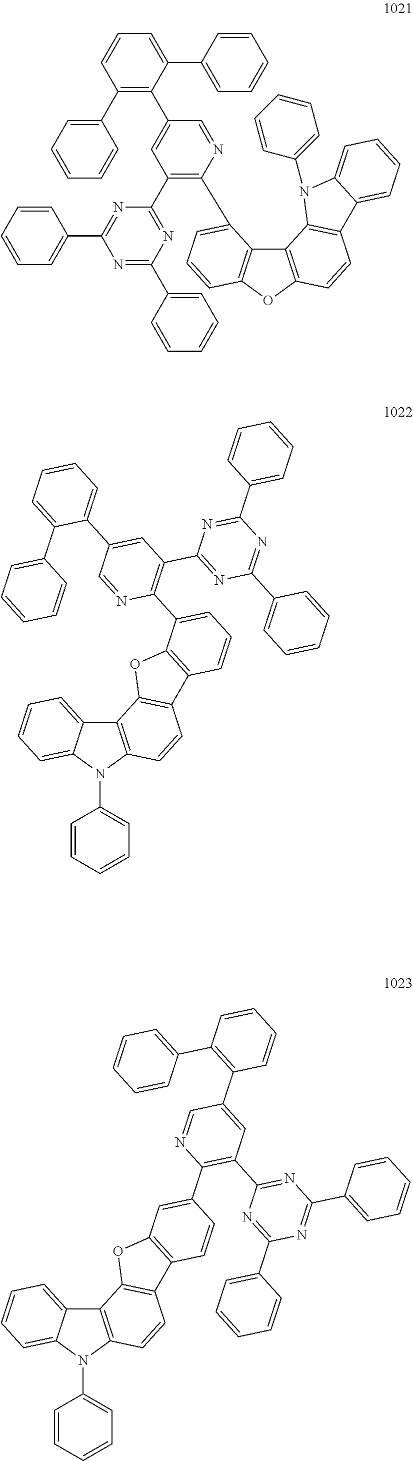 [SMILES: c1ccc(-c2nc(-c3ccccc3)nc(-c3cc(-c4c(-c5ccccc5)cccc4-c4ccccc4)cnc3-c3cccc4oc5ccc6c7ccccc7n(-c7ccccc7)c6c5c34)n2)cc1 ; c1ccc(-c2nc(-c3ccccc3)nc(-c3cc(-c4ccccc4-c4ccccc4)cnc3-c3cccc4c3oc3c4ccc4c3c3ccccc3n4-c3ccccc3)n2)cc1 ; c1ccc(-c2nc(-c3ccccc3)nc(-c3cc(-c4ccccc4-c4ccccc4)cnc3-c3ccc4c(c3)oc3c4ccc4c3c3ccccc3n4-c3ccccc3)n2)cc1]

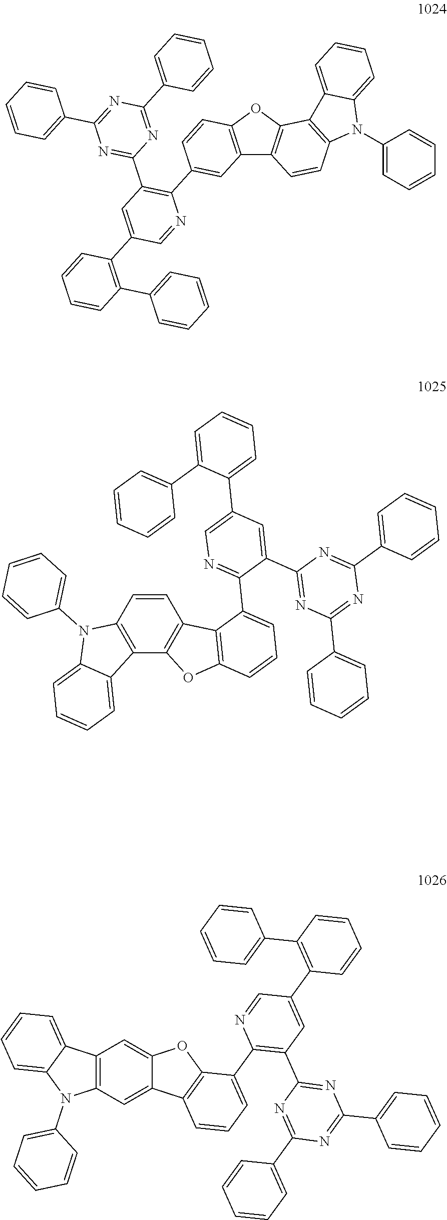 [SMILES: c1ccc(-c2nc(-c3ccccc3)nc(-c3cc(-c4ccccc4-c4ccccc4)cnc3-c3ccc4oc5c(ccc6c5c5ccccc5n6-c5ccccc5)c4c3)n2)cc1 ; c1ccc(-c2nc(-c3ccccc3)nc(-c3cc(-c4ccccc4-c4ccccc4)cnc3-c3cccc4oc5c(ccc6c5c5ccccc5n6-c5ccccc5)c34)n2)cc1 ; c1ccc(-c2nc(-c3ccccc3)nc(-c3cc(-c4ccccc4-c4ccccc4)cnc3-c3cccc4c3oc3cc5c6ccccc6n(-c6ccccc6)c5cc34)n2)cc1]

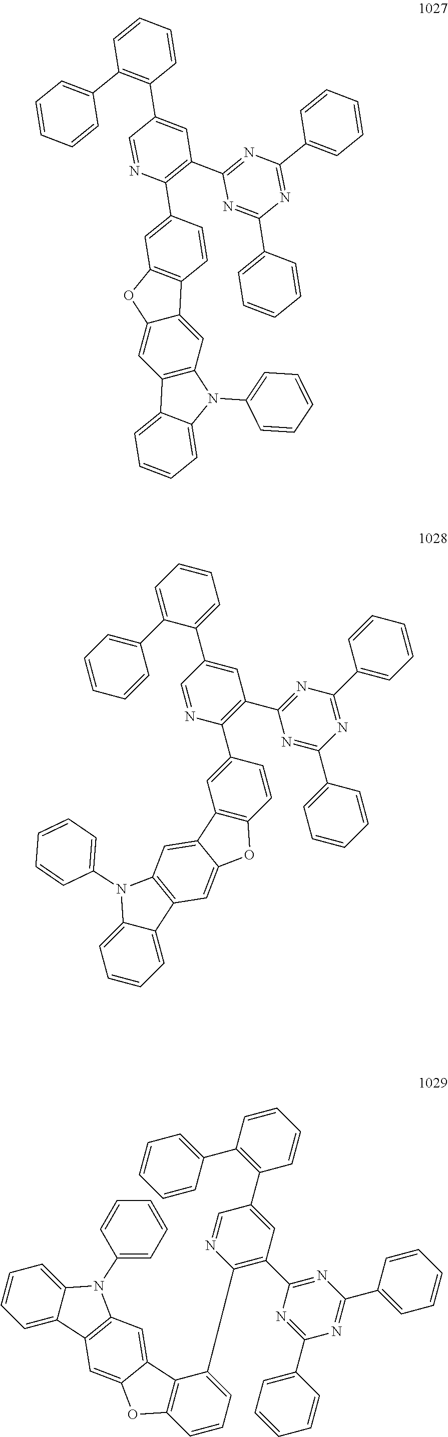 [SMILES: c1ccc(-c2nc(-c3ccccc3)nc(-c3cc(-c4ccccc4-c4ccccc4)cnc3-c3ccc4c(c3)oc3cc5c6ccccc6n(-c6ccccc6)c5cc34)n2)cc1 ; c1ccc(-c2nc(-c3ccccc3)nc(-c3cc(-c4ccccc4-c4ccccc4)cnc3-c3ccc4oc5cc6c7ccccc7n(-c7ccccc7)c6cc5c4c3)n2)cc1 ; c1ccc(-c2nc(-c3ccccc3)nc(-c3cc(-c4ccccc4-c4ccccc4)cnc3-c3cccc4oc5cc6c7ccccc7n(-c7ccccc7)c6cc5c34)n2)cc1]

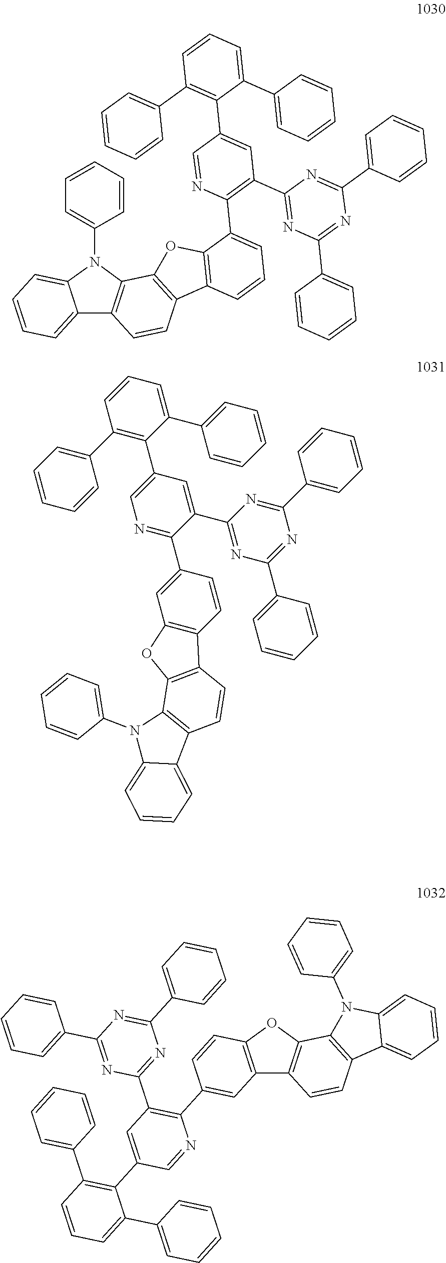

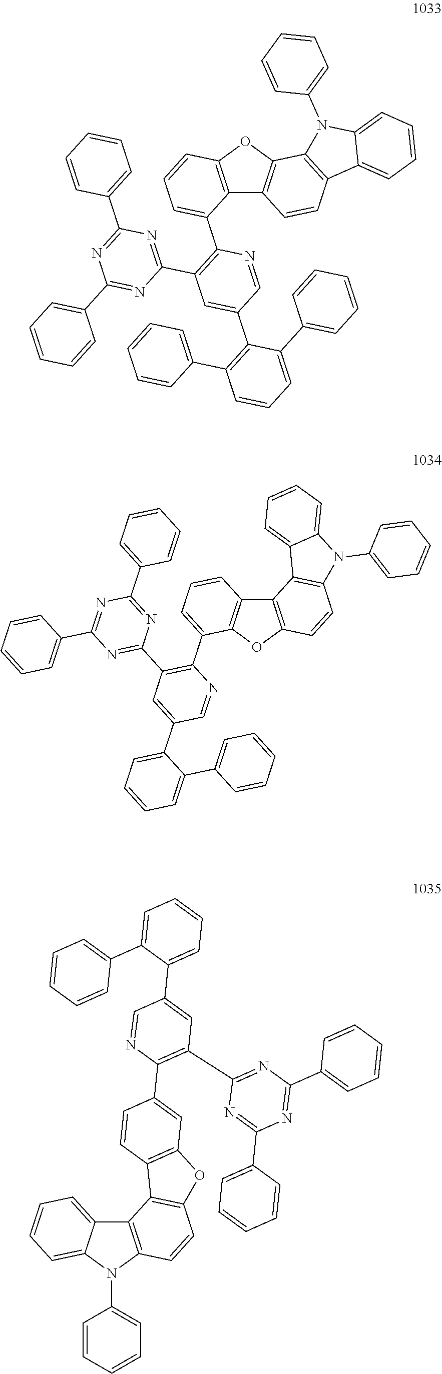 [SMILES: c1ccc(-c2nc(-c3ccccc3)nc(-c3cc(-c4c(-c5ccccc5)cccc4-c4ccccc4)cnc3-c3cccc4oc5c(ccc6c7ccccc7n(-c7ccccc7)c65)c34)n2)cc1 ; c1ccc(-c2nc(-c3ccccc3)nc(-c3cc(-c4ccccc4-c4ccccc4)cnc3-c3cccc4c3oc3ccc5c(c6ccccc6n5-c5ccccc5)c34)n2)cc1 ; c1ccc(-c2nc(-c3ccccc3)nc(-c3cc(-c4ccccc4-c4ccccc4)cnc3-c3ccc4c(c3)oc3ccc5c(c6ccccc6n5-c5ccccc5)c34)n2)cc1]

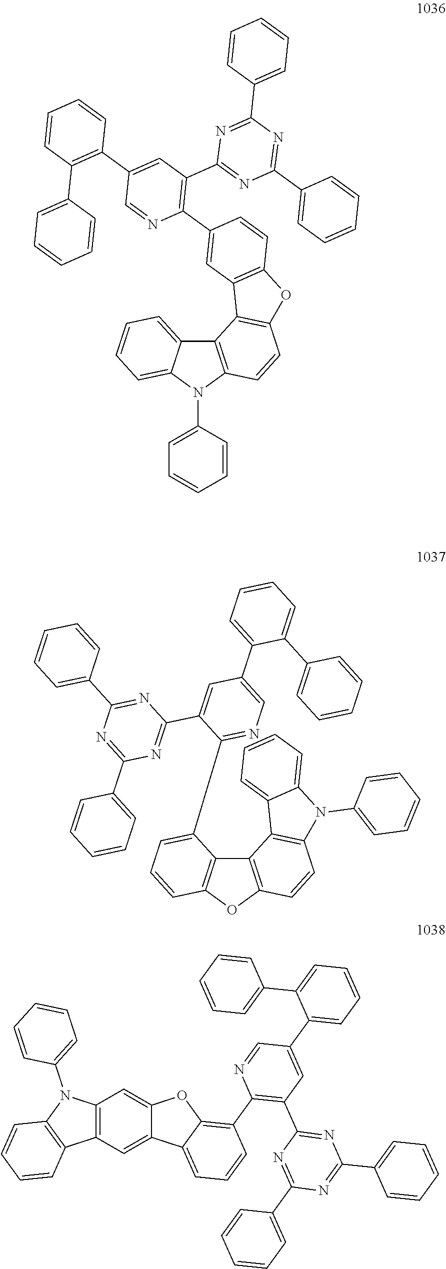 [SMILES: c1ccc(-c2nc(-c3ccccc3)nc(-c3cc(-c4ccccc4-c4ccccc4)cnc3-c3ccc4oc5ccc6c(c7ccccc7n6-c6ccccc6)c5c4c3)n2)cc1 ; c1ccc(-c2nc(-c3ccccc3)nc(-c3cc(-c4ccccc4-c4ccccc4)cnc3-c3cccc4oc5ccc6c(c7ccccc7n6-c6ccccc6)c5c34)n2)cc1 ; c1ccc(-c2nc(-c3ccccc3)nc(-c3cc(-c4ccccc4-c4ccccc4)cnc3-c3cccc4c3oc3cc5c(cc34)c3ccccc3n5-c3ccccc3)n2)cc1]

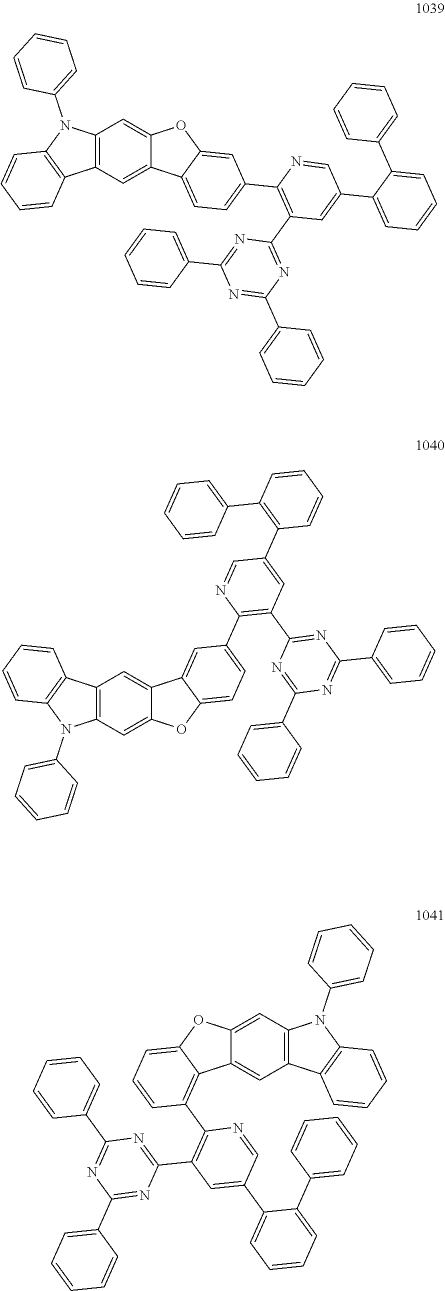 [SMILES: c1ccc(-c2nc(-c3ccccc3)nc(-c3cc(-c4ccccc4-c4ccccc4)cnc3-c3ccc4c(c3)oc3cc5c(cc34)c3ccccc3n5-c3ccccc3)n2)cc1 ; c1ccc(-c2nc(-c3ccccc3)nc(-c3cc(-c4ccccc4-c4ccccc4)cnc3-c3ccc4oc5cc6c(cc5c4c3)c3ccccc3n6-c3ccccc3)n2)cc1 ; c1ccc(-c2nc(-c3ccccc3)nc(-c3cc(-c4ccccc4-c4ccccc4)cnc3-c3cccc4oc5cc6c(cc5c34)c3ccccc3n6-c3ccccc3)n2)cc1]

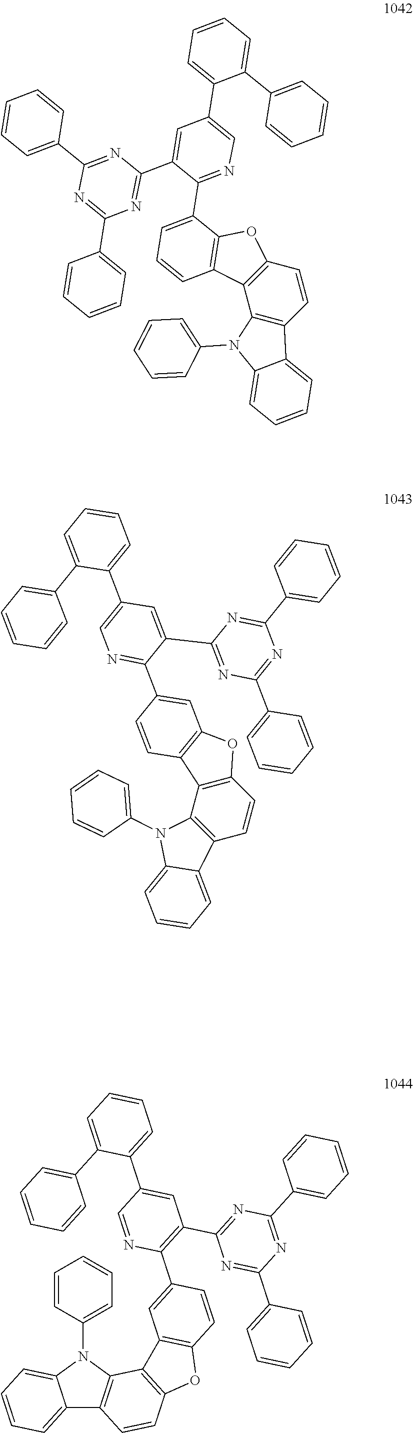 [SMILES: c1ccc(-c2nc(-c3ccccc3)nc(-c3cc(-c4ccccc4-c4ccccc4)cnc3-c3cccc4c3oc3ccc5c6ccccc6n(-c6ccccc6)c5c34)n2)cc1 ; c1ccc(-c2nc(-c3ccccc3)nc(-c3cc(-c4ccccc4-c4ccccc4)cnc3-c3ccc4c(c3)oc3ccc5c6ccccc6n(-c6ccccc6)c5c34)n2)cc1 ; c1ccc(-c2nc(-c3ccccc3)nc(-c3cc(-c4ccccc4-c4ccccc4)cnc3-c3ccc4oc5ccc6c7ccccc7n(-c7ccccc7)c6c5c4c3)n2)cc1]

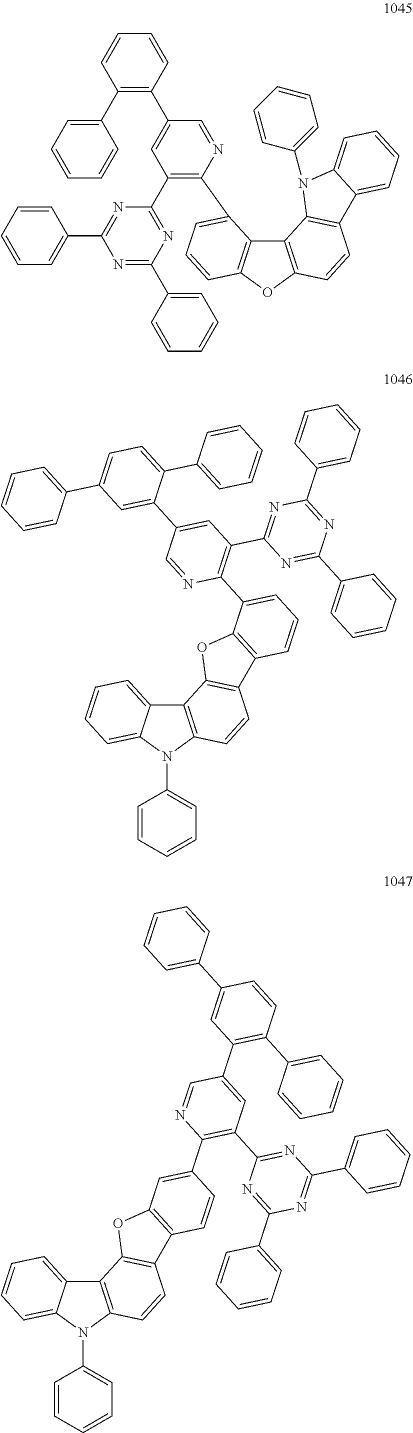 [SMILES: c1ccc(-c2nc(-c3ccccc3)nc(-c3cc(-c4ccccc4-c4ccccc4)cnc3-c3cccc4oc5ccc6c7ccccc7n(-c7ccccc7)c6c5c34)n2)cc1 ; c1ccc(-c2ccc(-c3ccccc3)c(-c3cnc(-c4cccc5c4oc4c5ccc5c4c4ccccc4n5-c4ccccc4)c(-c4nc(-c5ccccc5)nc(-c5ccccc5)n4)c3)c2)cc1 ; c1ccc(-c2ccc(-c3ccccc3)c(-c3cnc(-c4ccc5c(c4)oc4c5ccc5c4c4ccccc4n5-c4ccccc4)c(-c4nc(-c5ccccc5)nc(-c5ccccc5)n4)c3)c2)cc1]

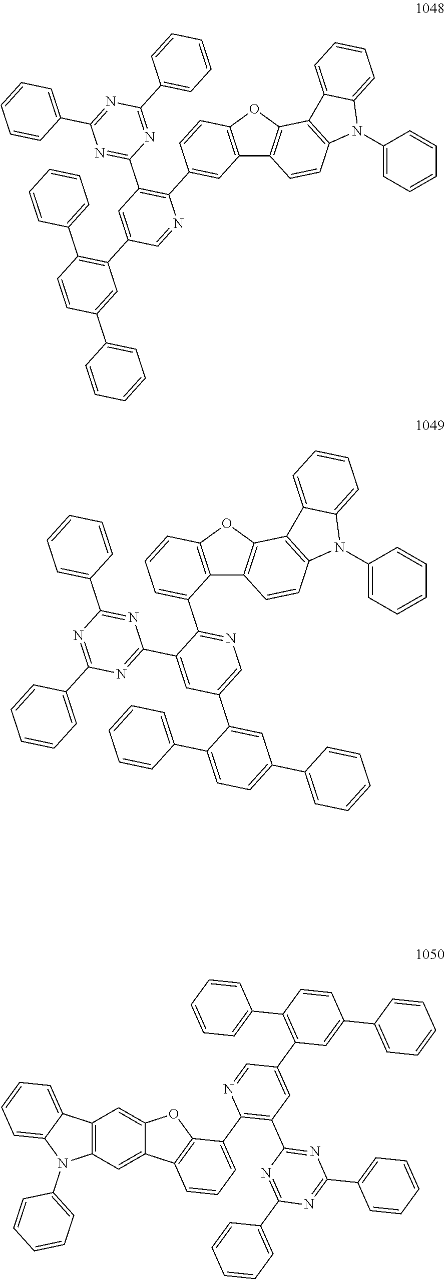 [SMILES: c1ccc(-c2ccc(-c3ccccc3)c(-c3cnc(-c4ccc5oc6c(ccc7c6c6ccccc6n7-c6ccccc6)c5c4)c(-c4nc(-c5ccccc5)nc(-c5ccccc5)n4)c3)c2)cc1 ; c1ccc(-c2ccc(-c3ccccc3)c(-c3cnc(-c4cccc5oc6c(ccc7c6c6ccccc6n7-c6ccccc6)c45)c(-c4nc(-c5ccccc5)nc(-c5ccccc5)n4)c3)c2)cc1 ; c1ccc(-c2ccc(-c3ccccc3)c(-c3cnc(-c4cccc5c4oc4cc6c7ccccc7n(-c7ccccc7)c6cc45)c(-c4nc(-c5ccccc5)nc(-c5ccccc5)n4)c3)c2)cc1]

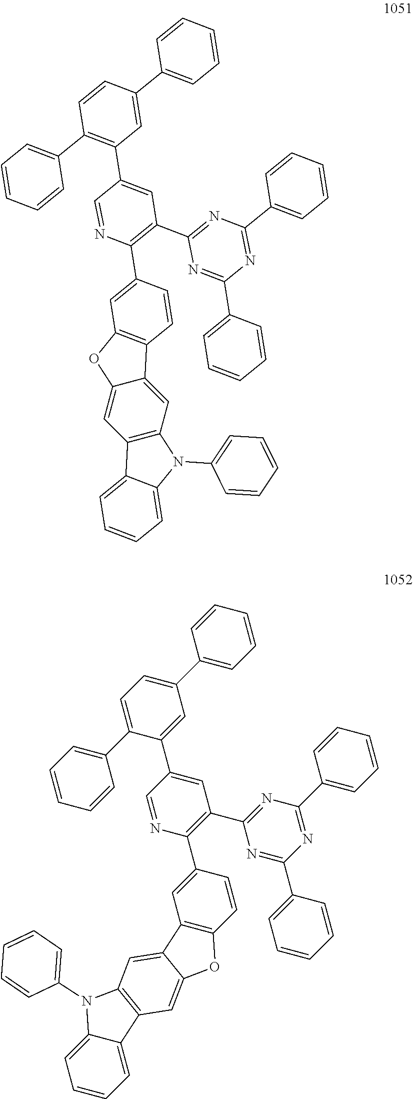 [SMILES: c1ccc(-c2ccc(-c3ccccc3)c(-c3cnc(-c4ccc5c(c4)oc4cc6c7ccccc7n(-c7ccccc7)c6cc45)c(-c4nc(-c5ccccc5)nc(-c5ccccc5)n4)c3)c2)cc1 ; c1ccc(-c2ccc(-c3ccccc3)c(-c3cnc(-c4ccc5oc6cc7c8ccccc8n(-c8ccccc8)c7cc6c5c4)c(-c4nc(-c5ccccc5)nc(-c5ccccc5)n4)c3)c2)cc1]

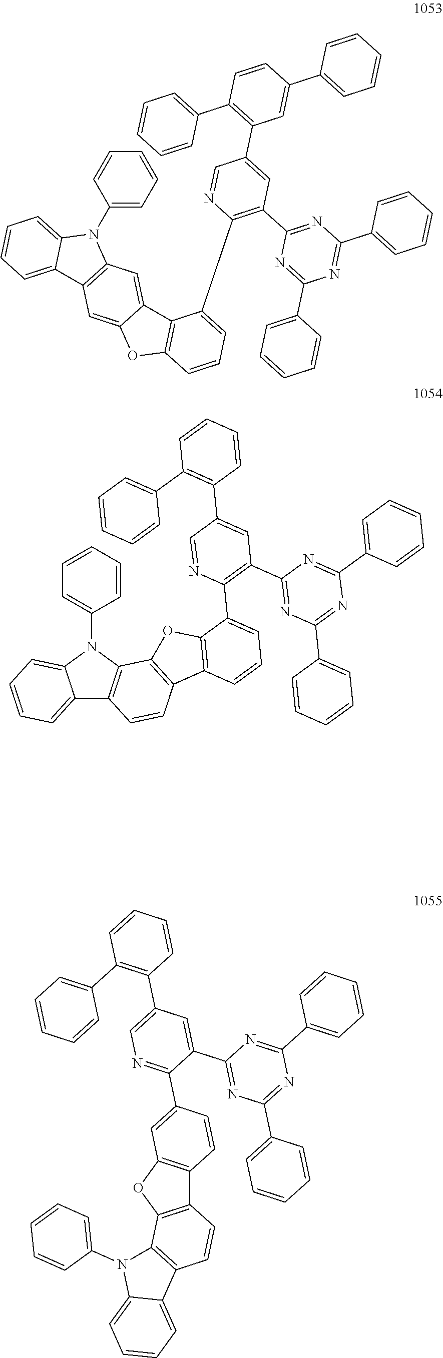 [SMILES: c1ccc(-c2ccc(-c3ccccc3)c(-c3cnc(-c4cccc5oc6cc7c8ccccc8n(-c8ccccc8)c7cc6c45)c(-c4nc(-c5ccccc5)nc(-c5ccccc5)n4)c3)c2)cc1 ; c1ccc(-c2nc(-c3ccccc3)nc(-c3cc(-c4ccccc4-c4ccccc4)cnc3-c3cccc4c3oc3c4ccc4c5ccccc5n(-c5ccccc5)c43)n2)cc1 ; c1ccc(-c2nc(-c3ccccc3)nc(-c3cc(-c4ccccc4-c4ccccc4)cnc3-c3ccc4c(c3)oc3c4ccc4c5ccccc5n(-c5ccccc5)c43)n2)cc1]

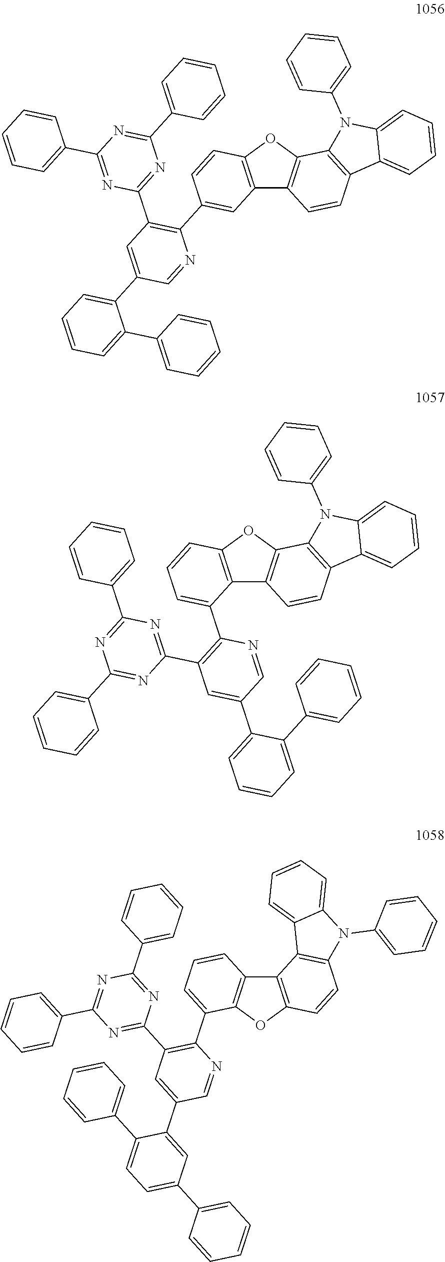 [SMILES: c1ccc(-c2nc(-c3ccccc3)nc(-c3cc(-c4ccccc4-c4ccccc4)cnc3-c3ccc4oc5c(ccc6c7ccccc7n(-c7ccccc7)c65)c4c3)n2)cc1 ; c1ccc(-c2nc(-c3ccccc3)nc(-c3cc(-c4ccccc4-c4ccccc4)cnc3-c3cccc4oc5c(ccc6c7ccccc7n(-c7ccccc7)c65)c34)n2)cc1 ; c1ccc(-c2ccc(-c3ccccc3)c(-c3cnc(-c4cccc5c4oc4ccc6c(c7ccccc7n6-c6ccccc6)c45)c(-c4nc(-c5ccccc5)nc(-c5ccccc5)n4)c3)c2)cc1]

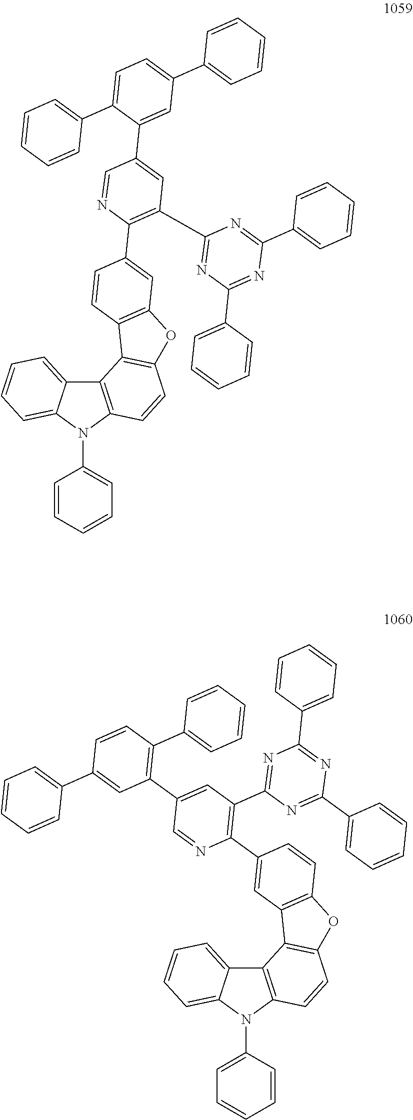 [SMILES: c1ccc(-c2ccc(-c3ccccc3)c(-c3cnc(-c4ccc5c(c4)oc4ccc6c(c7ccccc7n6-c6ccccc6)c45)c(-c4nc(-c5ccccc5)nc(-c5ccccc5)n4)c3)c2)cc1 ; c1ccc(-c2ccc(-c3ccccc3)c(-c3cnc(-c4ccc5oc6ccc7c(c8ccccc8n7-c7ccccc7)c6c5c4)c(-c4nc(-c5ccccc5)nc(-c5ccccc5)n4)c3)c2)cc1]

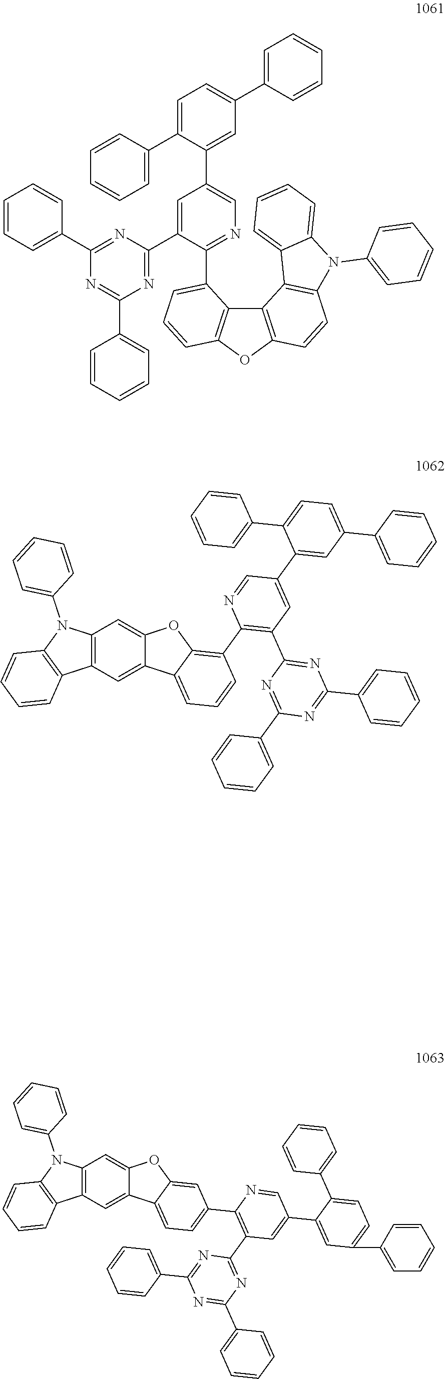 [SMILES: c1ccc(-c2ccc(-c3ccccc3)c(-c3cnc(-c4cccc5oc6ccc7c(c8ccccc8n7-c7ccccc7)c6c45)c(-c4nc(-c5ccccc5)nc(-c5ccccc5)n4)c3)c2)cc1 ; c1ccc(-c2ccc(-c3ccccc3)c(-c3cnc(-c4cccc5c4oc4cc6c(cc45)c4ccccc4n6-c4ccccc4)c(-c4nc(-c5ccccc5)nc(-c5ccccc5)n4)c3)c2)cc1 ; c1ccc(-c2ccc(-c3ccccc3)c(-c3cnc(-c4ccc5c(c4)oc4cc6c(cc45)c4ccccc4n6-c4ccccc4)c(-c4nc(-c5ccccc5)nc(-c5ccccc5)n4)c3)c2)cc1]

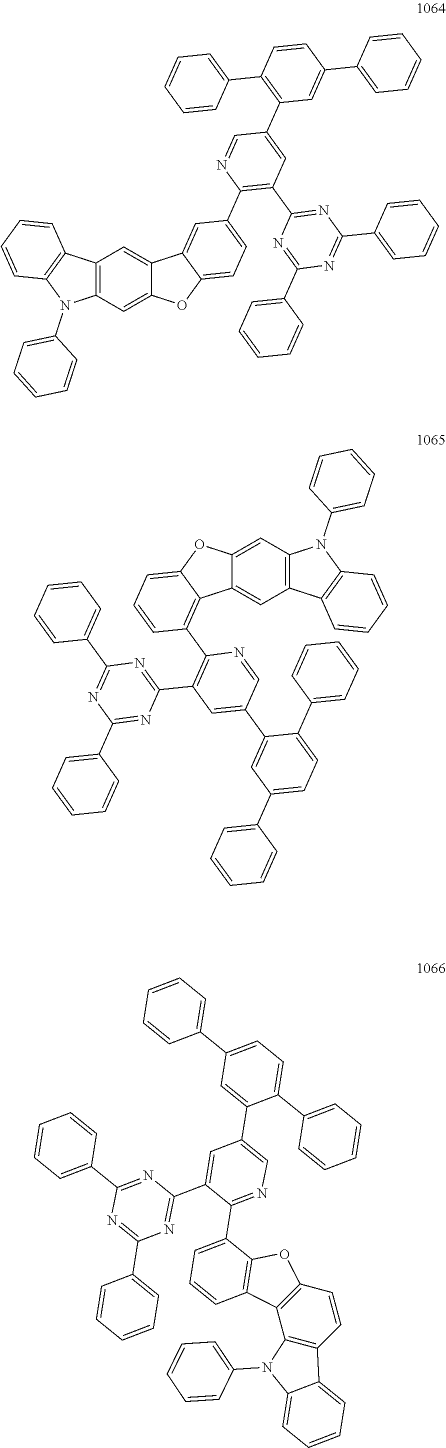 [SMILES: c1ccc(-c2ccc(-c3ccccc3)c(-c3cnc(-c4ccc5oc6cc7c(cc6c5c4)c4ccccc4n7-c4ccccc4)c(-c4nc(-c5ccccc5)nc(-c5ccccc5)n4)c3)c2)cc1 ; c1ccc(-c2ccc(-c3ccccc3)c(-c3cnc(-c4cccc5oc6cc7c(cc6c45)c4ccccc4n7-c4ccccc4)c(-c4nc(-c5ccccc5)nc(-c5ccccc5)n4)c3)c2)cc1 ; c1ccc(-c2ccc(-c3ccccc3)c(-c3cnc(-c4cccc5c4oc4ccc6c7ccccc7n(-c7ccccc7)c6c45)c(-c4nc(-c5ccccc5)nc(-c5ccccc5)n4)c3)c2)cc1]

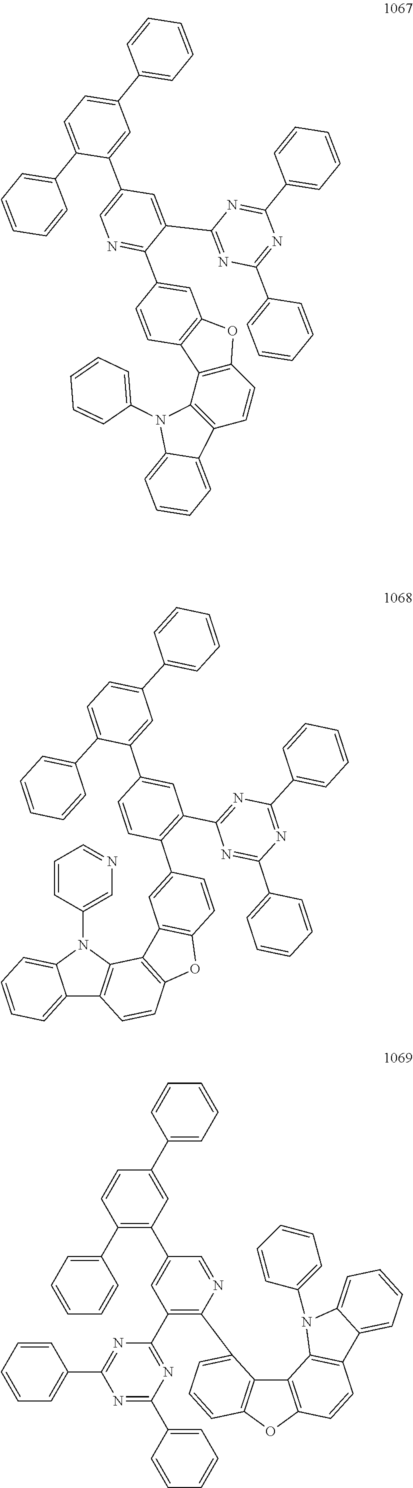 [SMILES: c1ccc(-c2ccc(-c3ccccc3)c(-c3cnc(-c4ccc5c(c4)oc4ccc6c7ccccc7n(-c7ccccc7)c6c45)c(-c4nc(-c5ccccc5)nc(-c5ccccc5)n4)c3)c2)cc1 ; c1ccc(-c2ccc(-c3ccccc3)c(-c3ccc(-c4ccc5oc6ccc7c8ccccc8n(-c8cccnc8)c7c6c5c4)c(-c4nc(-c5ccccc5)nc(-c5ccccc5)n4)c3)c2)cc1 ; c1ccc(-c2ccc(-c3ccccc3)c(-c3cnc(-c4cccc5oc6ccc7c8ccccc8n(-c8ccccc8)c7c6c45)c(-c4nc(-c5ccccc5)nc(-c5ccccc5)n4)c3)c2)cc1]

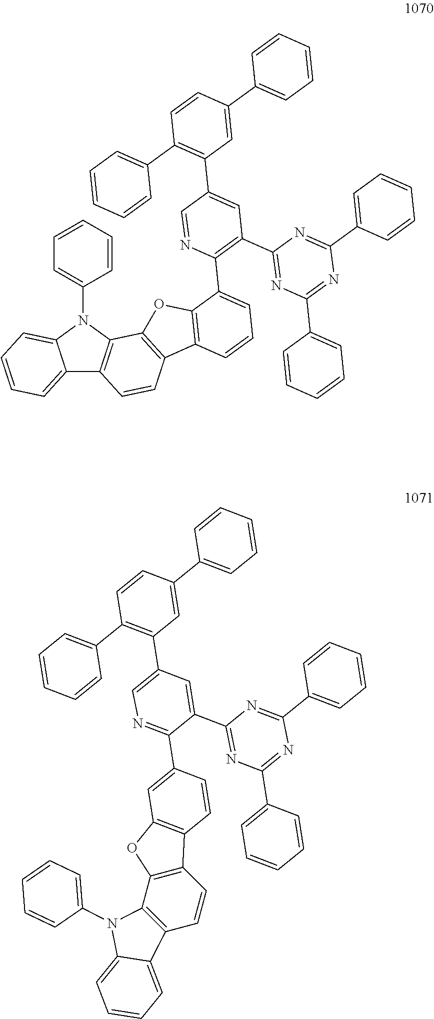 [SMILES: c1ccc(-c2ccc(-c3ccccc3)c(-c3cnc(-c4cccc5c4oc4c5ccc5c6ccccc6n(-c6ccccc6)c54)c(-c4nc(-c5ccccc5)nc(-c5ccccc5)n4)c3)c2)cc1 ; c1ccc(-c2ccc(-c3ccccc3)c(-c3cnc(-c4ccc5c(c4)oc4c5ccc5c6ccccc6n(-c6ccccc6)c54)c(-c4nc(-c5ccccc5)nc(-c5ccccc5)n4)c3)c2)cc1]

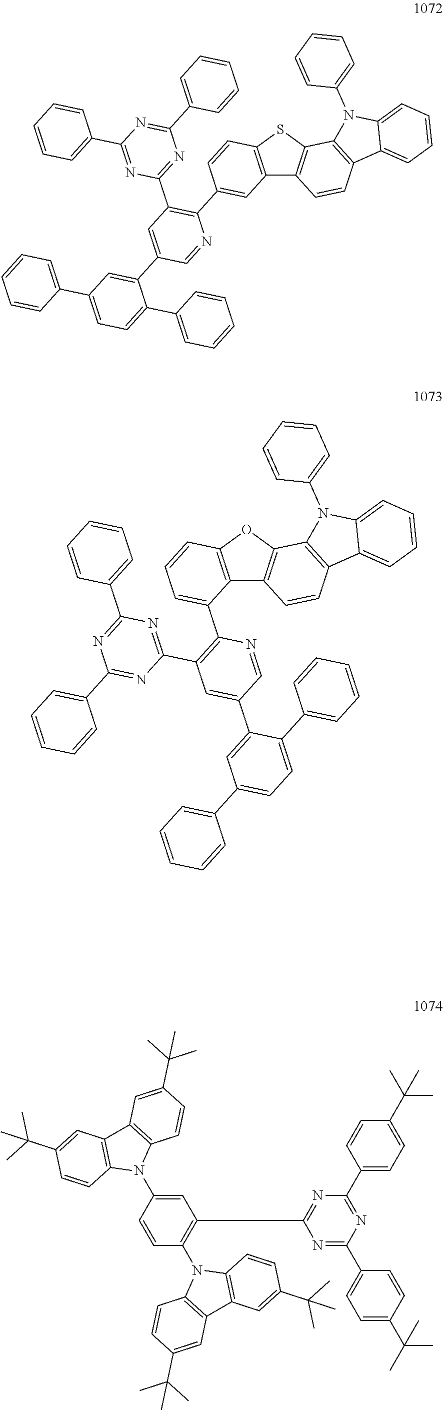 [SMILES: c1ccc(-c2ccc(-c3ccccc3)c(-c3cnc(-c4ccc5sc6c(ccc7c8ccccc8n(-c8ccccc8)c76)c5c4)c(-c4nc(-c5ccccc5)nc(-c5ccccc5)n4)c3)c2)cc1 ; c1ccc(-c2ccc(-c3ccccc3)c(-c3cnc(-c4cccc5oc6c(ccc7c8ccccc8n(-c8ccccc8)c76)c45)c(-c4nc(-c5ccccc5)nc(-c5ccccc5)n4)c3)c2)cc1 ; CC(C)(C)c1ccc(-c2nc(-c3ccc(C(C)(C)C)cc3)nc(-c3cc(-n4c5ccc(C(C)(C)C)cc5c5cc(C(C)(C)C)ccc54)ccc3-n3c4ccc(C(C)(C)C)cc4c4cc(C(C)(C)C)ccc43)n2)cc1]

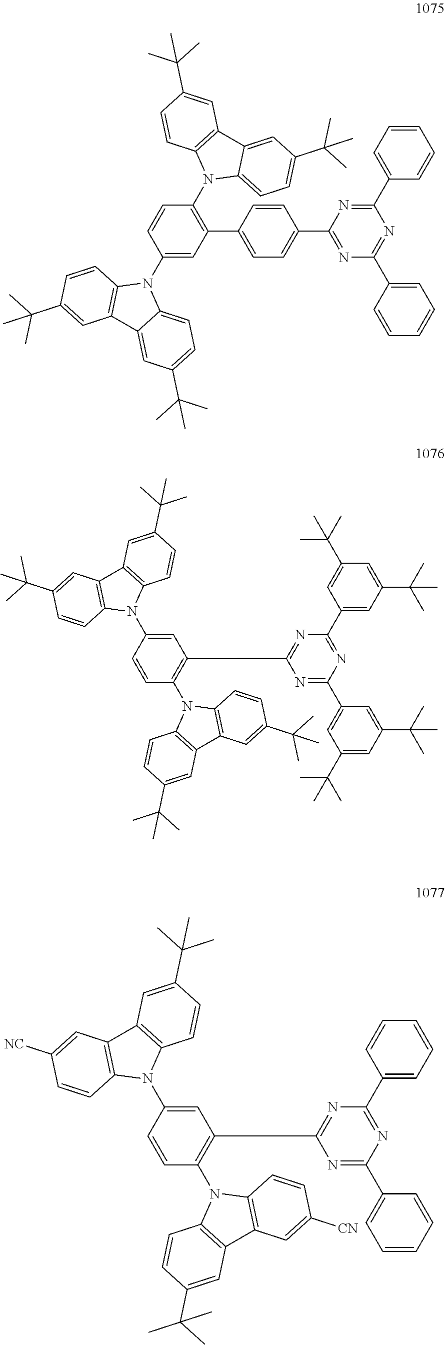 [SMILES: CC(C)(C)c1ccc2c(c1)c1cc(C(C)(C)C)ccc1n2-c1ccc(-n2c3ccc(C(C)(C)C)cc3c3cc(C(C)(C)C)ccc32)c(-c2ccc(-c3nc(-c4ccccc4)nc(-c4ccccc4)n3)cc2)c1 ; CC(C)(C)c1cc(-c2nc(-c3cc(C(C)(C)C)cc(C(C)(C)C)c3)nc(-c3cc(-n4c5ccc(C(C)(C)C)cc5c5cc(C(C)(C)C)ccc54)ccc3-n3c4ccc(C(C)(C)C)cc4c4cc(C(C)(C)C)ccc43)n2)cc(C(C)(C)C)c1 ; CC(C)(C)c1ccc2c(c1)c1cc(C#N)ccc1n2-c1ccc(-n2c3ccc(C#N)cc3c3cc(C(C)(C)C)ccc32)c(-c2nc(-c3ccccc3)nc(-c3ccccc3)n2)c1]

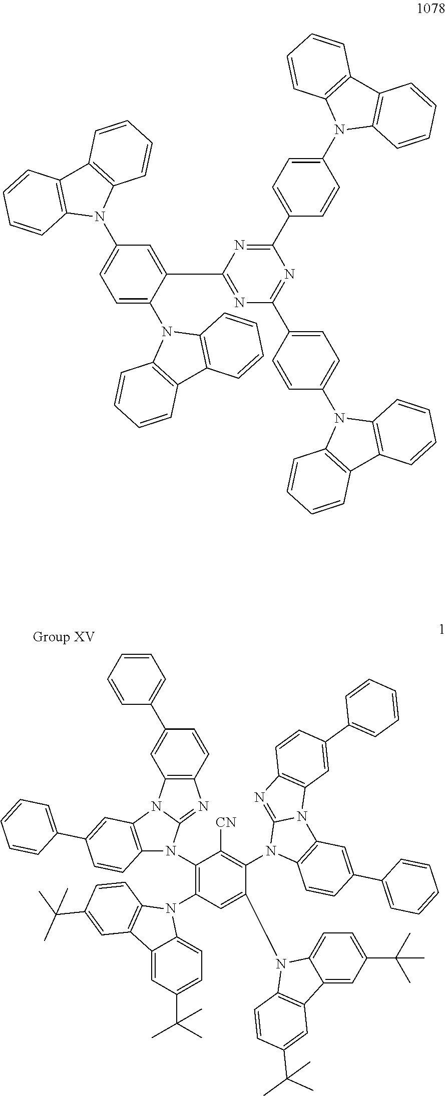 [SMILES: c1ccc2c(c1)c1ccccc1n2-c1ccc(-c2nc(-c3ccc(-n4c5ccccc5c5ccccc54)cc3)nc(-c3cc(-n4c5ccccc5c5ccccc54)ccc3-n3c4ccccc4c4ccccc43)n2)cc1 ; CC(C)(C)c1ccc2c(c1)c1cc(C(C)(C)C)ccc1n2-c1cc(-n2c3ccc(C(C)(C)C)cc3c3cc(C(C)(C)C)ccc32)c(-n2c3ccc(-c4ccccc4)cc3n3c4cc(-c5ccccc5)ccc4nc23)c(C#N)c1-n1c2ccc(-c3ccccc3)cc2n2c3cc(-c4ccccc4)ccc3nc12]

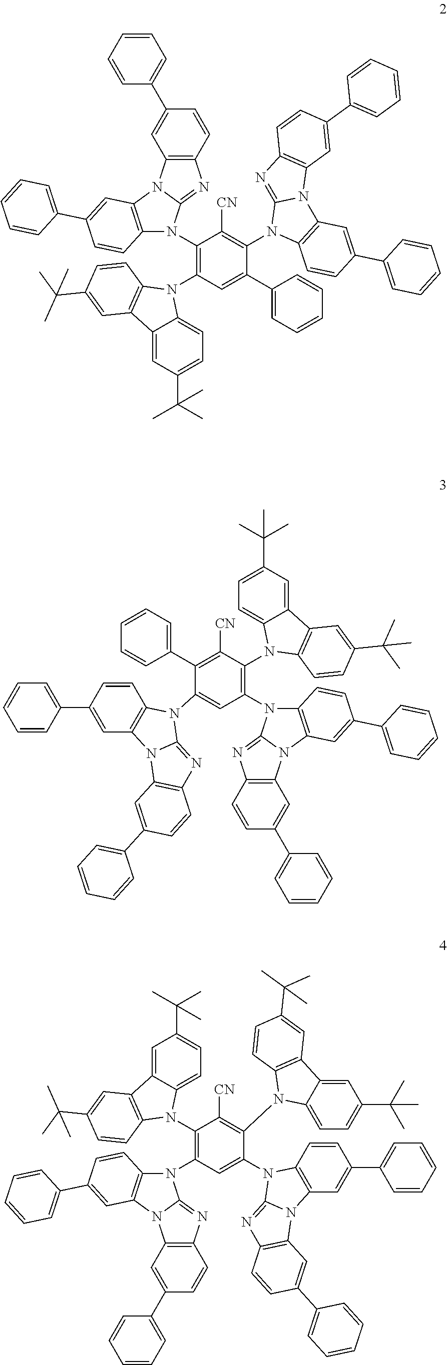

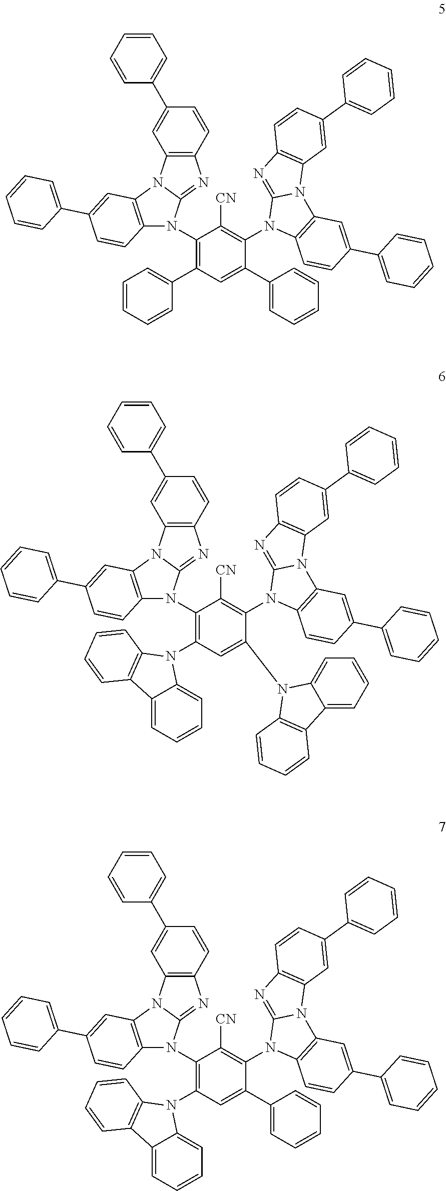 [SMILES: N#Cc1c(-n2c3ccc(-c4ccccc4)cc3n3c4cc(-c5ccccc5)ccc4nc23)c(-c2ccccc2)cc(-c2ccccc2)c1-n1c2ccc(-c3ccccc3)cc2n2c3cc(-c4ccccc4)ccc3nc12 ; N#Cc1c(-n2c3ccc(-c4ccccc4)cc3n3c4cc(-c5ccccc5)ccc4nc23)c(-n2c3ccccc3c3ccccc32)cc(-n2c3ccccc3c3ccccc32)c1-n1c2ccc(-c3ccccc3)cc2n2c3cc(-c4ccccc4)ccc3nc12 ; N#Cc1c(-n2c3ccc(-c4ccccc4)cc3n3c4cc(-c5ccccc5)ccc4nc23)c(-c2ccccc2)cc(-n2c3ccccc3c3ccccc32)c1-n1c2ccc(-c3ccccc3)cc2n2c3cc(-c4ccccc4)ccc3nc12]

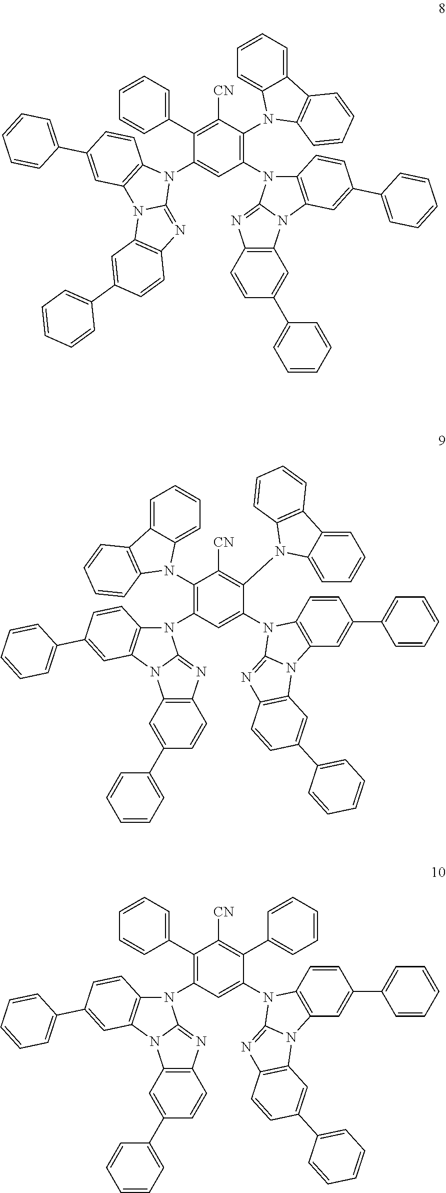 [SMILES: N#Cc1c(-c2ccccc2)c(-n2c3ccc(-c4ccccc4)cc3n3c4cc(-c5ccccc5)ccc4nc23)cc(-n2c3ccc(-c4ccccc4)cc3n3c4cc(-c5ccccc5)ccc4nc23)c1-n1c2ccccc2c2ccccc21 ; N#Cc1c(-n2c3ccccc3c3ccccc32)c(-n2c3ccc(-c4ccccc4)cc3n3c4cc(-c5ccccc5)ccc4nc23)cc(-n2c3ccc(-c4ccccc4)cc3n3c4cc(-c5ccccc5)ccc4nc23)c1-n1c2ccccc2c2ccccc21 ; N#Cc1c(-c2ccccc2)c(-n2c3ccc(-c4ccccc4)cc3n3c4cc(-c5ccccc5)ccc4nc23)cc(-n2c3ccc(-c4ccccc4)cc3n3c4cc(-c5ccccc5)ccc4nc23)c1-c1ccccc1]

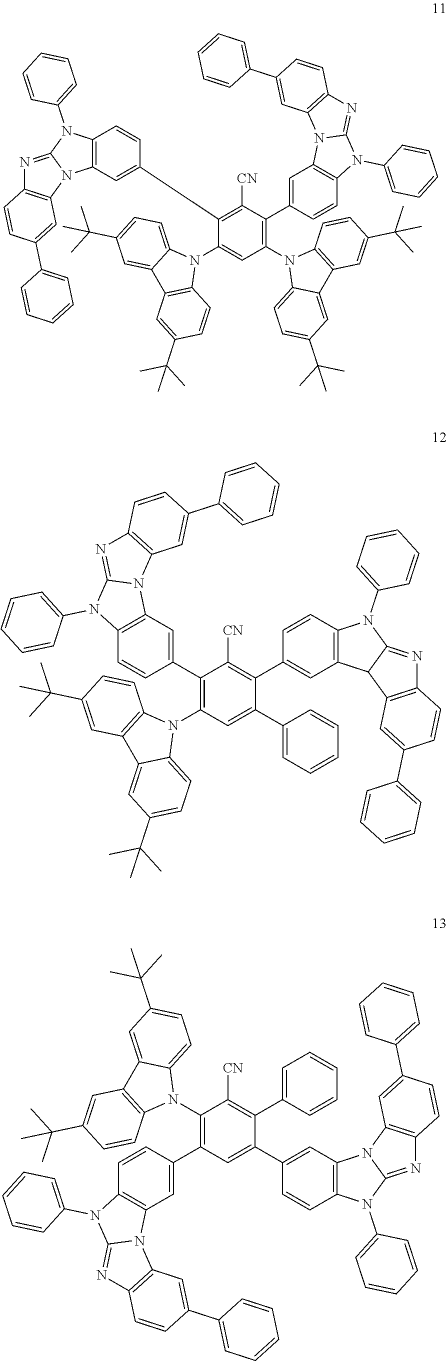

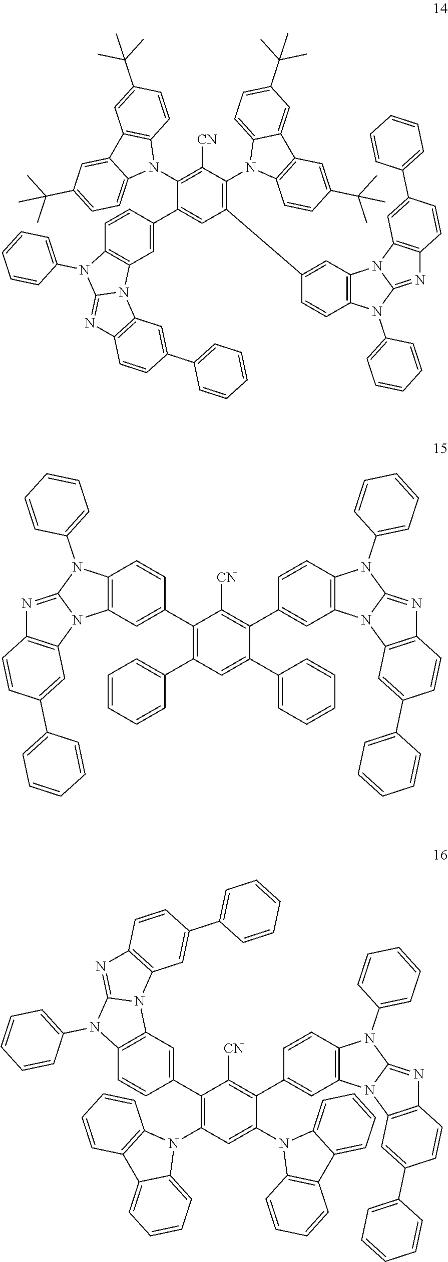 [SMILES: CC(C)(C)c1ccc2c(c1)c1cc(C(C)(C)C)ccc1n2-c1c(-c2ccc3c(c2)n2c4cc(-c5ccccc5)ccc4nc2n3-c2ccccc2)cc(-c2ccc3c(c2)n2c4cc(-c5ccccc5)ccc4nc2n3-c2ccccc2)c(-n2c3ccc(C(C)(C)C)cc3c3cc(C(C)(C)C)ccc32)c1C#N ; N#Cc1c(-c2ccc3c(c2)n2c4cc(-c5ccccc5)ccc4nc2n3-c2ccccc2)c(-c2ccccc2)cc(-c2ccccc2)c1-c1ccc2c(c1)n1c3cc(-c4ccccc4)ccc3nc1n2-c1ccccc1 ; N#Cc1c(-c2ccc3c(c2)n2c4cc(-c5ccccc5)ccc4nc2n3-c2ccccc2)c(-n2c3ccccc3c3ccccc32)cc(-n2c3ccccc3c3ccccc32)c1-c1ccc2c(c1)n1c3cc(-c4ccccc4)ccc3nc1n2-c1ccccc1]

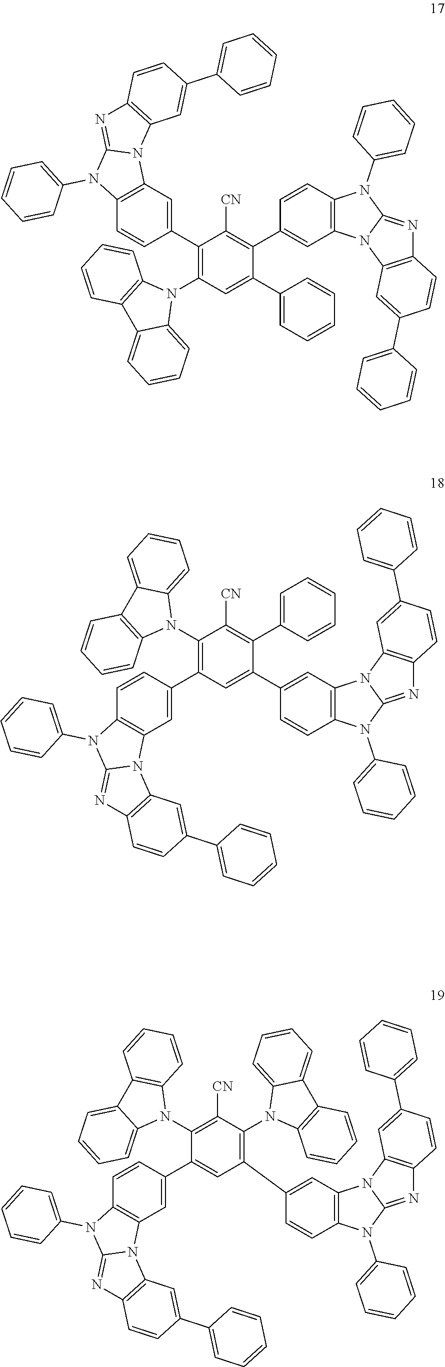 [SMILES: N#Cc1c(-c2ccc3c(c2)n2c4cc(-c5ccccc5)ccc4nc2n3-c2ccccc2)c(-c2ccccc2)cc(-n2c3ccccc3c3ccccc32)c1-c1ccc2c(c1)n1c3cc(-c4ccccc4)ccc3nc1n2-c1ccccc1 ; N#Cc1c(-c2ccccc2)c(-c2ccc3c(c2)n2c4cc(-c5ccccc5)ccc4nc2n3-c2ccccc2)cc(-c2ccc3c(c2)n2c4cc(-c5ccccc5)ccc4nc2n3-c2ccccc2)c1-n1c2ccccc2c2ccccc21 ; N#Cc1c(-n2c3ccccc3c3ccccc32)c(-c2ccc3c(c2)n2c4cc(-c5ccccc5)ccc4nc2n3-c2ccccc2)cc(-c2ccc3c(c2)n2c4cc(-c5ccccc5)ccc4nc2n3-c2ccccc2)c1-n1c2ccccc2c2ccccc21]

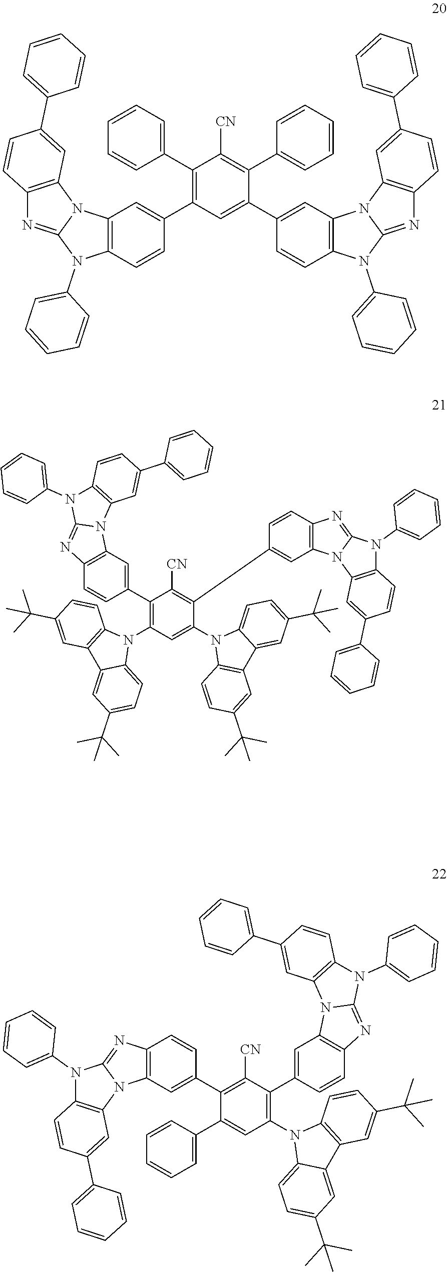 [SMILES: N#Cc1c(-c2ccccc2)c(-c2ccc3c(c2)n2c4cc(-c5ccccc5)ccc4nc2n3-c2ccccc2)cc(-c2ccc3c(c2)n2c4cc(-c5ccccc5)ccc4nc2n3-c2ccccc2)c1-c1ccccc1 ; CC(C)(C)c1ccc2c(c1)c1cc(C(C)(C)C)ccc1n2-c1cc(-n2c3ccc(C(C)(C)C)cc3c3cc(C(C)(C)C)ccc32)c(-c2ccc3nc4n(-c5ccccc5)c5ccc(-c6ccccc6)cc5n4c3c2)c(C#N)c1-c1ccc2nc3n(-c4ccccc4)c4ccc(-c5ccccc5)cc4n3c2c1 ; CC(C)(C)c1ccc2c(c1)c1cc(C(C)(C)C)ccc1n2-c1cc(-c2ccccc2)c(-c2ccc3nc4n(-c5ccccc5)c5ccc(-c6ccccc6)cc5n4c3c2)c(C#N)c1-c1ccc2nc3n(-c4ccccc4)c4ccc(-c5ccccc5)cc4n3c2c1]

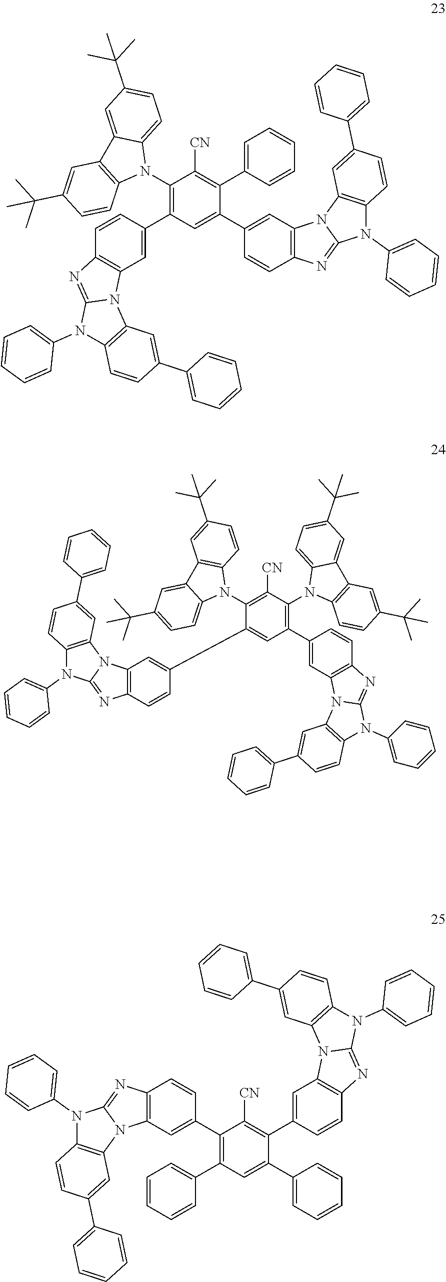 [SMILES: CC(C)(C)c1ccc2c(c1)c1cc(C(C)(C)C)ccc1n2-c1c(-c2ccc3nc4n(-c5ccccc5)c5ccc(-c6ccccc6)cc5n4c3c2)cc(-c2ccc3nc4n(-c5ccccc5)c5ccc(-c6ccccc6)cc5n4c3c2)c(-c2ccccc2)c1C#N ; CC(C)(C)c1ccc2c(c1)c1cc(C(C)(C)C)ccc1n2-c1c(-c2ccc3nc4n(-c5ccccc5)c5ccc(-c6ccccc6)cc5n4c3c2)cc(-c2ccc3nc4n(-c5ccccc5)c5ccc(-c6ccccc6)cc5n4c3c2)c(-n2c3ccc(C(C)(C)C)cc3c3cc(C(C)(C)C)ccc32)c1C#N ; N#Cc1c(-c2ccc3nc4n(-c5ccccc5)c5ccc(-c6ccccc6)cc5n4c3c2)c(-c2ccccc2)cc(-c2ccccc2)c1-c1ccc2nc3n(-c4ccccc4)c4ccc(-c5ccccc5)cc4n3c2c1]

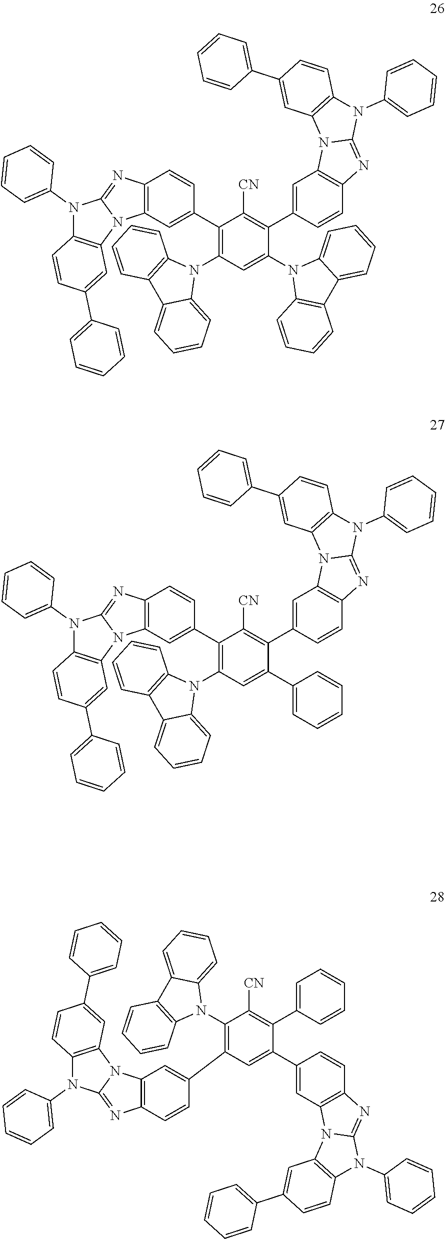 [SMILES: N#Cc1c(-c2ccc3nc4n(-c5ccccc5)c5ccc(-c6ccccc6)cc5n4c3c2)c(-n2c3ccccc3c3ccccc32)cc(-n2c3ccccc3c3ccccc32)c1-c1ccc2nc3n(-c4ccccc4)c4ccc(-c5ccccc5)cc4n3c2c1 ; N#Cc1c(-c2ccc3nc4n(-c5ccccc5)c5ccc(-c6ccccc6)cc5n4c3c2)c(-c2ccccc2)cc(-n2c3ccccc3c3ccccc32)c1-c1ccc2nc3n(-c4ccccc4)c4ccc(-c5ccccc5)cc4n3c2c1 ; N#Cc1c(-c2ccccc2)c(-c2ccc3nc4n(-c5ccccc5)c5ccc(-c6ccccc6)cc5n4c3c2)cc(-c2ccc3nc4n(-c5ccccc5)c5ccc(-c6ccccc6)cc5n4c3c2)c1-n1c2ccccc2c2ccccc21]

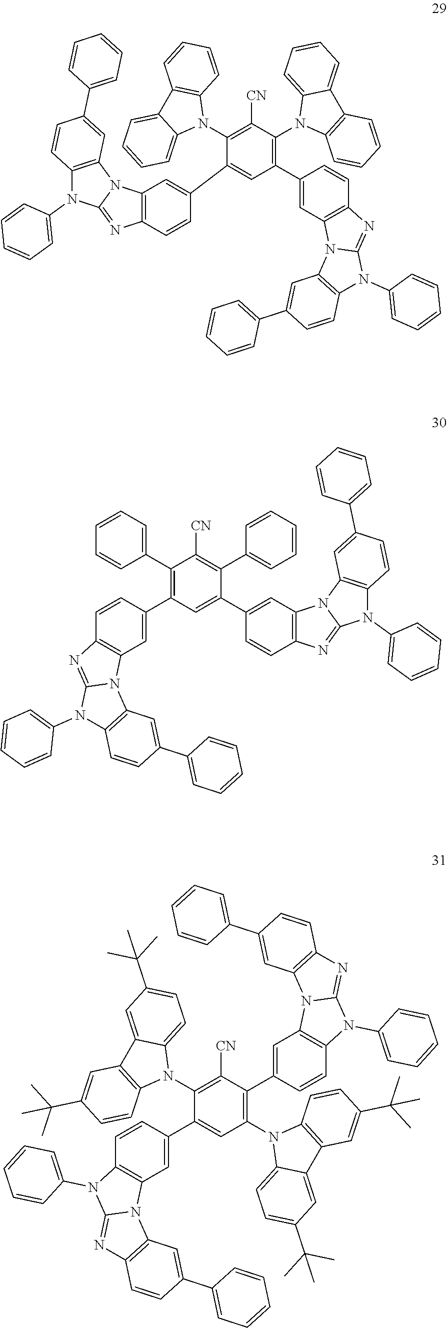 [SMILES: N#Cc1c(-n2c3ccccc3c3ccccc32)c(-c2ccc3nc4n(-c5ccccc5)c5ccc(-c6ccccc6)cc5n4c3c2)cc(-c2ccc3nc4n(-c5ccccc5)c5ccc(-c6ccccc6)cc5n4c3c2)c1-n1c2ccccc2c2ccccc21 ; N#Cc1c(-c2ccccc2)c(-c2ccc3nc4n(-c5ccccc5)c5ccc(-c6ccccc6)cc5n4c3c2)cc(-c2ccc3nc4n(-c5ccccc5)c5ccc(-c6ccccc6)cc5n4c3c2)c1-c1ccccc1 ; CC(C)(C)c1ccc2c(c1)c1cc(C(C)(C)C)ccc1n2-c1cc(-c2ccc3c(c2)n2c4cc(-c5ccccc5)ccc4nc2n3-c2ccccc2)c(-n2c3ccc(C(C)(C)C)cc3c3cc(C(C)(C)C)ccc32)c(C#N)c1-c1ccc2c(c1)n1c3cc(-c4ccccc4)ccc3nc1n2-c1ccccc1]

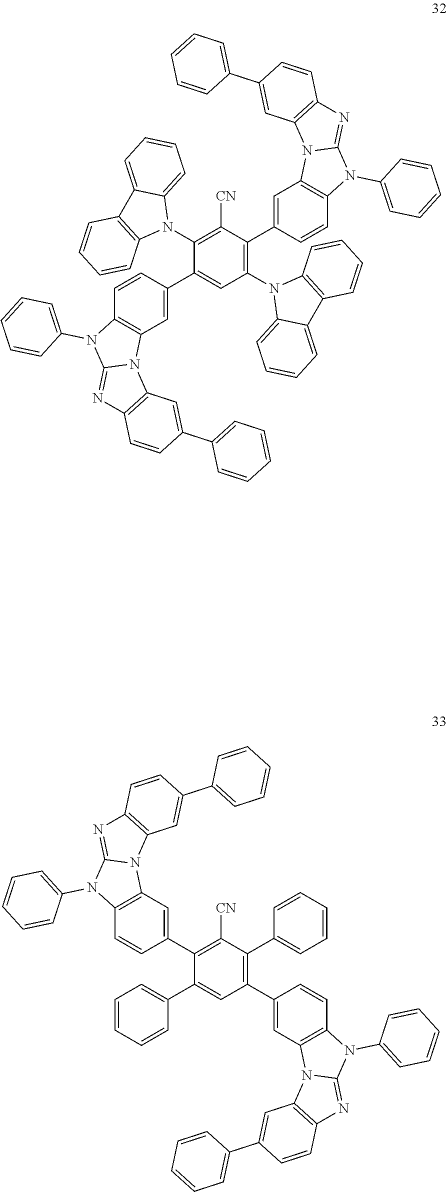 [SMILES: N#Cc1c(-c2ccc3c(c2)n2c4cc(-c5ccccc5)ccc4nc2n3-c2ccccc2)c(-n2c3ccccc3c3ccccc32)cc(-c2ccc3c(c2)n2c4cc(-c5ccccc5)ccc4nc2n3-c2ccccc2)c1-n1c2ccccc2c2ccccc21 ; N#Cc1c(-c2ccccc2)c(-c2ccc3c(c2)n2c4cc(-c5ccccc5)ccc4nc2n3-c2ccccc2)cc(-c2ccccc2)c1-c1ccc2c(c1)n1c3cc(-c4ccccc4)ccc3nc1n2-c1ccccc1]

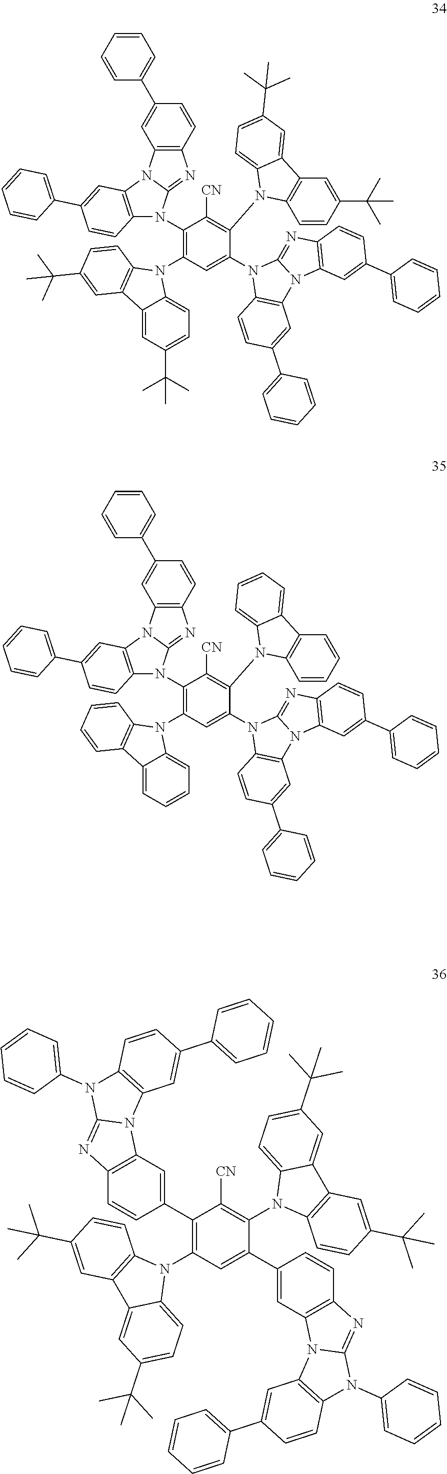

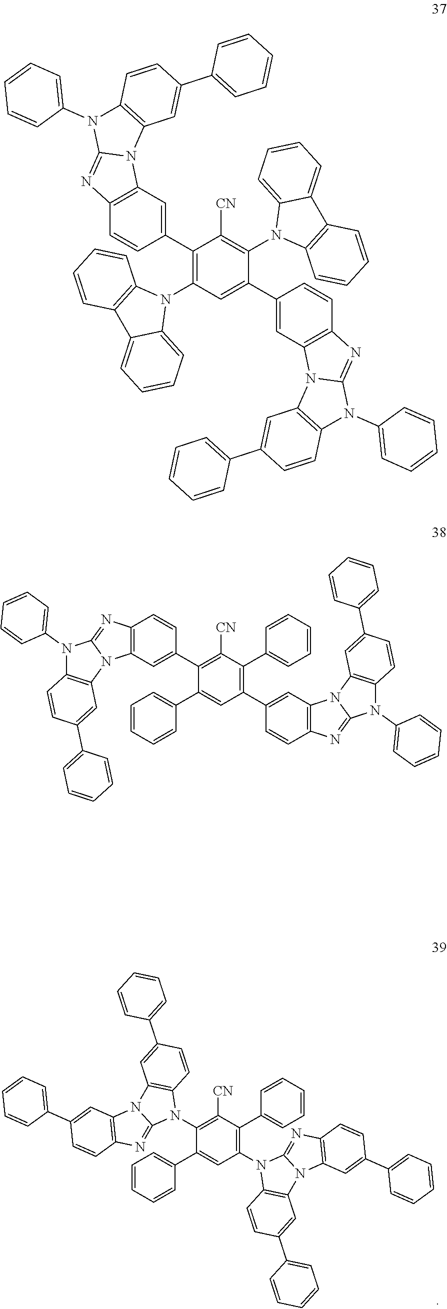 [SMILES: N#Cc1c(-c2ccc3nc4n(-c5ccccc5)c5ccc(-c6ccccc6)cc5n4c3c2)c(-n2c3ccccc3c3ccccc32)cc(-c2ccc3nc4n(-c5ccccc5)c5ccc(-c6ccccc6)cc5n4c3c2)c1-n1c2ccccc2c2ccccc21 ; N#Cc1c(-c2ccccc2)c(-c2ccc3nc4n(-c5ccccc5)c5ccc(-c6ccccc6)cc5n4c3c2)cc(-c2ccccc2)c1-c1ccc2nc3n(-c4ccccc4)c4ccc(-c5ccccc5)cc4n3c2c1 ; N#Cc1c(-c2ccccc2)c(-n2c3ccc(-c4ccccc4)cc3n3c4cc(-c5ccccc5)ccc4nc23)cc(-c2ccccc2)c1-n1c2ccc(-c3ccccc3)cc2n2c3cc(-c4ccccc4)ccc3nc12]

Hole Transport Region 12

The hole transport region 12 may be arranged between the first electrode 11 and the emission layer 15 of the organic light-emitting device 10.

The hole transport region 12 may have a single-layer structure or a multi-layer structure.

For example, the hole transport region 12 may have a hole injection layer, a hole transport layer, a hole injection layer/hole transport layer structure, a hole injection layer/first hole transport layer/second hole transport layer structure, a hole transport layer/interlayer structure, a hole injection layer/hole transport layer/interlayer structure, a hole transport layer/electron blocking layer structure, or a hole injection layer/hole transport layer/electron blocking layer structure, but embodiments of the present disclosure are not limited thereto.

The hole transport region 12 may include any compound having hole-transporting properties.

For example, the hole transport region 12 may include an amine-based compound.

In an embodiment, the hole transport region 1 may include at least one a compound represented by Formula 201 to a compound represented by Formula 205, but embodiments of the present disclosure are not limited thereto:

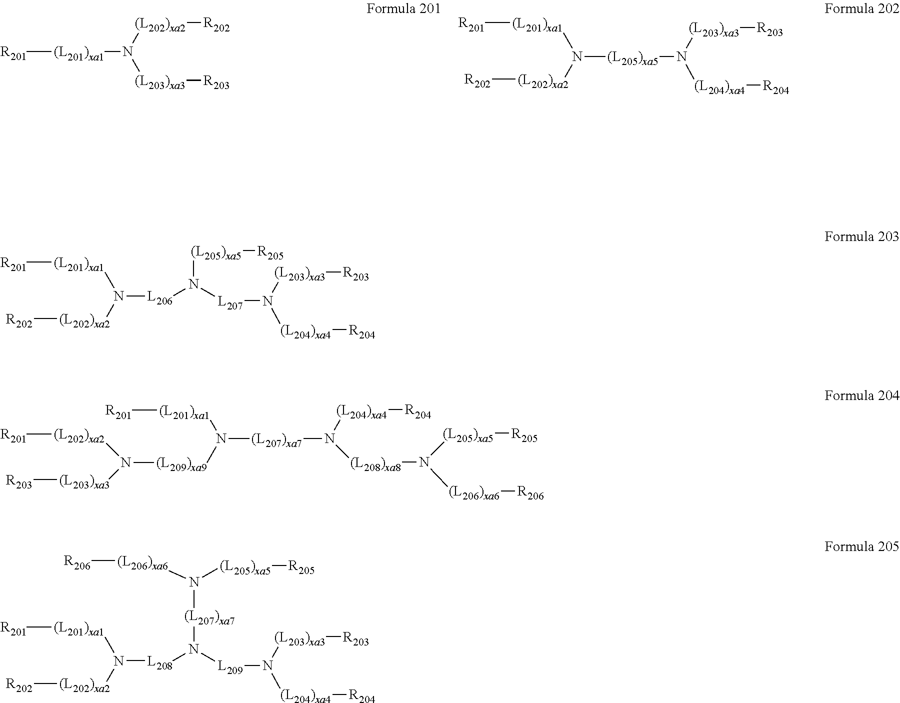

wherein, in Formulae 201 to 205,
L₂₀₁ to L₂₀₉ may each independently *-be O—*′, *—S—*′, a substituted or unsubstituted
C₅-C₆₀ carbocyclic group, or a substituted or unsubstituted C₁-C₆₀ heterocyclic group,
xa1 to xa9 may each independently be an integer from 0 to 5, and
R₂₀₁ to R₂₀₆ may each independently be a substituted or unsubstituted C₃-C₁₀ cycloalkyl group, a substituted or unsubstituted heterocycloalkyl group, a substituted or unsubstituted C₃-C₁₀ cycloalkenyl group, a substituted or unsubstituted C₂-C₁₀ heterocycloalkenyl group, a substituted or unsubstituted C₆-C₆₀ aryl group, a substituted or unsubstituted C₆-C₆₀ aryloxy group, a substituted or unsubstituted C₆-C₆₀ arylthio group, a substituted or unsubstituted heteroaryl group, a substituted or unsubstituted monovalent aromatic condensed polycyclic group, a substituted or unsubstituted monovalent aromatic condensed heteropolycyclic group, a substituted or unsubstituted monovalent non-aromatic condensed polycyclic group, and a substituted or unsubstituted monovalent non-aromatic condensed heteropolycyclic group, and two adjacent groups R₂₀₁ to R₂₀₆ may optionally be bound via a single bond, a dimethyl-methylene group, or a diphenyl-methylene group.

For example, L₂₀₁ to L₂₀₉ may be:
a benzene group, a heptalene group, an indene group, a naphthalene group, an azulene group, a heptalene group, an indacene group, an acenaphthylene group, a fluorene group, a spiro-bifluorene group, a benzofluorene group, a dibenzofluorene group, a phenalene group, a phenanthrene group, an anthracene group, a fluoranthene group, a triphenylene group, a pyrene group, a chrysene group, a naphthacene group, a picene group, a perylene group, a pentacene group, a hexacene group, a pentacene group, a rubicene group, a coragen group, an ovalene group, a pyrrole group, an isoindole group, an indole group, a furan group, a thiophene group, a benzofuran group, a benzothiophene group, a benzocarbazole group, a dibenzocarbazole group, a dibenzofuran group, a dibenzothiophene group, a dibenzothiophene sulfone group, a carbazole group, a dibenzosilole group, an indenocarbazole group, an indolocarbazole group, a benzofurocarbazole group, a benzothienocarbazole group, and a triindolobenzene group, each unsubstituted or substituted with deuterium, a C₁-C₁₀ alkyl group, a C₁-C₁₀ alkoxy group, a phenyl group, a naphthyl group, a fluorenyl group, a carbazolyl group, a dibenzofuranyl group, a dibenzothiophenyl group, a triphenylenyl group, a biphenyl group, a terphenyl group, a tetraphenyl group, or —Si(Q₁₁)(Q₁₂)(Q₁₃),
xa1 to xa9 may each independently be 0, 1, or 2, and
R₂₀₁ to R₂₀₆ may each independently be a phenyl group, a biphenyl group, a terphenyl group, a pentalenyl group, an indenyl group, a naphthyl group, an azulenyl group, a heptalenyl group, an indacenyl group, an acenaphthyl group, a fluorenyl group, a spiro-bifluorenyl group, a benzofluorenyl group, a dibenzofluorenyl group, a phenalenyl group, a phenanthrenyl group, an anthracenyl group, a fluoranthenyl group, a triphenylenyl group, a pyrenyl group, a chrysenyl group, a naphthacenyl group, a picenyl group, a perylenyl group, a pentaphenyl group, a hexacenyl group, a pentacenyl group, a rubicenyl group, a coronenyl group, an ovalenyl group, a thiophenyl group, a furanyl group, a carbazolyl group, an indolyl group, an isoindolyl group, a benzofuranyl group, a benzothiophenyl group, a dibenzofuranyl group, a dibenzothiophenyl group, a benzocarbazolyl group, a dibenzocarbazolyl group, a dibenzosilolyl group, a pyridinyl group, an indeno carbazolyl group, an indolocarbazolyl group, a benzofurocarbazolyl group, or a benzothienocarbazolyl group, each unsubstituted or substituted with deuterium, —F, —Cl, —Br, —I, a hydroxyl group, a cyano group, a nitro group, an amidino group, a hydrazine group, a hydrazone group, a C₁-C₂₀ alkyl group, a C₁-C₂₀ alkoxy group, a cyclopentyl group, a cyclohexyl group, a cycloheptyl group, a cyclopentenyl group, a cyclohexenyl group, a phenyl group, a biphenyl group, a terphenyl group, a phenyl group substituted with a C₁-C₁₀ alkyl group, a phenyl group substituted with —F, a pentalenyl group, an indenyl group, a naphthyl group, an azulenyl group, a heptalenyl group, an indacenyl group, an acenaphthyl group, a fluorenyl group, a spiro-bifluorenyl group, a benzofluorenyl group, a dibenzofluorenyl group, a phenalenyl group, a phenanthrenyl group, an anthracenyl group, a fluoranthenyl group, a triphenylenyl group, a pyrenyl group, a chrysenyl group, a naphthacenyl group, a picenyl group, a perylenyl group, a pentaphenyl group, a hexacenyl group, a pentacenyl group, a rubicenyl group, a coronenyl group, an ovalenyl group, a thiophenyl group, a furanyl group, a carbazolyl group, an indolyl group, an isoindolyl group, a benzofuranyl group, a benzothiophenyl group, a dibenzofuranyl group, a dibenzothiophenyl group, a benzocarbazolyl group, a dibenzocarbazolyl group, a dibenzosilolyl group, a pyridinyl group, —Si(Q₃₁)(Q₃₂)(Q₃₃), —N(Q₃₁)(Q₃₂), or a combination thereof,
wherein Q₁₁ to Q₁₃ and Q₃₁ to Q₃₃ may each independently be a C₁-C₁₀ alkyl group, a C₁-C₁₀ alkoxy group, a phenyl group, a biphenyl group, a terphenyl group, or a naphthyl group.

In one or more embodiments, the hole transport region 12 may include a carbazole-containing amine-based compound.

In an embodiment, the hole transport region 12 may include a carbazole-containing amine-based compound or a carbazole-free amine-based compound.

The carbazole-containing amine-based compound may be, for example, a compound represented by Formula 201 including a carbazole group and further including at least one of a dibenzofuran group, a dibenzothiophene group, a fluorene group, a spiro-bifluorene group, an indenocarbazole group, an indolocarbazole group, a benzofurocarbazole group, a benzothienocarbazole group, or a combination thereof.

The carbazole-free amine-based compound may be, for example, a compound represented by Formula 201 which do not include a carbazole group and which include at least one a dibenzofuran group, a dibenzothiophene group, a fluorene group, a spiro-bifluorene group, an indenocarbazole group, an indolocarbazole group, a benzofurocarbazole group, a benzothienocarbazole group, or a combination thereof.

In one or more embodiments, the hole transport region 12 may include at least one compound represented by Formulae 201 or 202.

In an embodiment, the hole transport region 12 may include at least one compound represented by Formulae 201-1, 202-1 or 201-2, but embodiments of the present disclosure are not limited thereto:

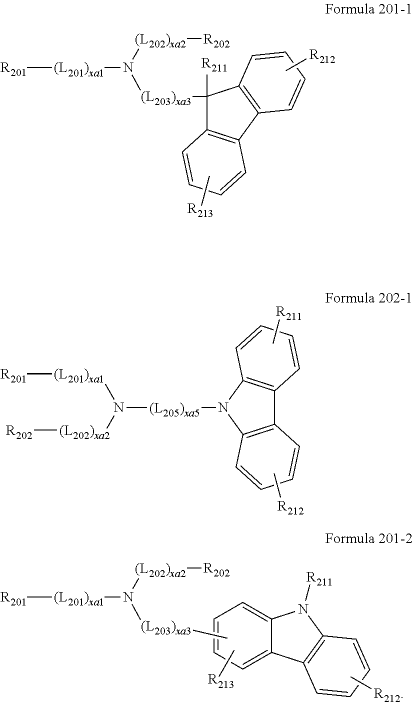

In Formulae 201-1, 202-1, and 201-2, L₂₀₁ to L₂₀₃, L₂₀₅, xa1 to xa3, xa5, R₂₀₁ and R₂₀₂ are the same as described herein, and R₂₁₁ to R₂₁₃ are each independently hydrogen, deuterium, —F, —Cl, —Br, —I, a hydroxyl group, a cyano group, a nitro group, an amidino group, a hydrazine group, a hydrazone group, a C₁-C₂₀ alkyl group, a C₁-C₂₀ alkoxy group, a phenyl group, a biphenyl group, a terphenyl group, a phenyl group substituted with a C₁-C₁₀ alkyl group, a phenyl group substituted with —F, a naphthyl group, a fluorenyl group, a spiro-bifluorenyl group, a dimethylfluorenyl group, a diphenylfluorenyl group, a triphenylenyl group, a thiophenyl group, a furanyl group, a carbazolyl group, an indolyl group, an isoindolyl group, a benzofuranyl group, a benzothiophenyl group, a dibenzofuranyl group, a dibenzothiophenyl group, a benzocarbazolyl group, a dibenzocarbazolyl group, a dibenzosilolyl group, a pyridinyl group, or a combination thereof.

For example, the hole transport region 12 may include at least one of Compounds HT1 to HT39, but embodiments of the present disclosure are not limited thereto.

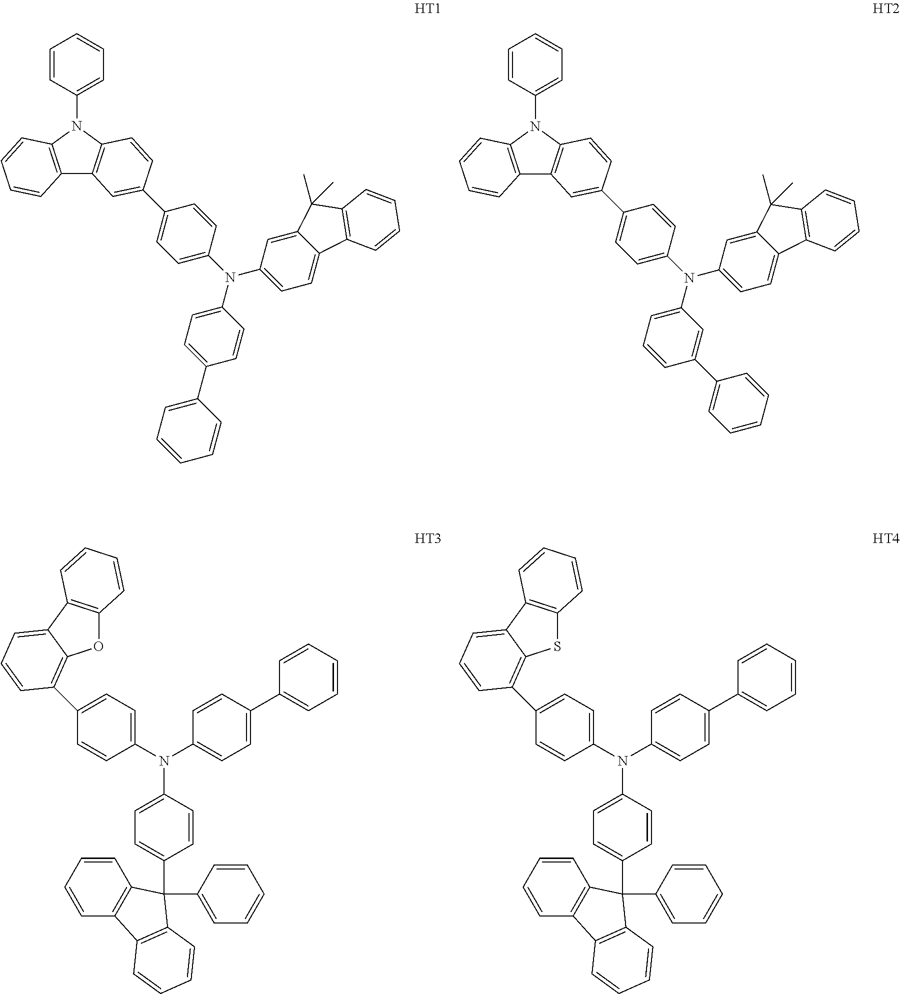 [SMILES: CC1(C)c2ccccc2-c2ccc(N(c3ccc(-c4ccccc4)cc3)c3ccc(-c4ccc5c(c4)c4ccccc4n5-c4ccccc4)cc3)cc21 ; CC1(C)c2ccccc2-c2ccc(N(c3ccc(-c4ccc5c(c4)c4ccccc4n5-c4ccccc4)cc3)c3cccc(-c4ccccc4)c3)cc21 ; c1ccc(-c2ccc(N(c3ccc(-c4cccc5c4oc4ccccc45)cc3)c3ccc(C4(c5ccccc5)c5ccccc5-c5ccccc54)cc3)cc2)cc1 ; c1ccc(-c2ccc(N(c3ccc(-c4cccc5c4sc4ccccc45)cc3)c3ccc(C4(c5ccccc5)c5ccccc5-c5ccccc54)cc3)cc2)cc1]

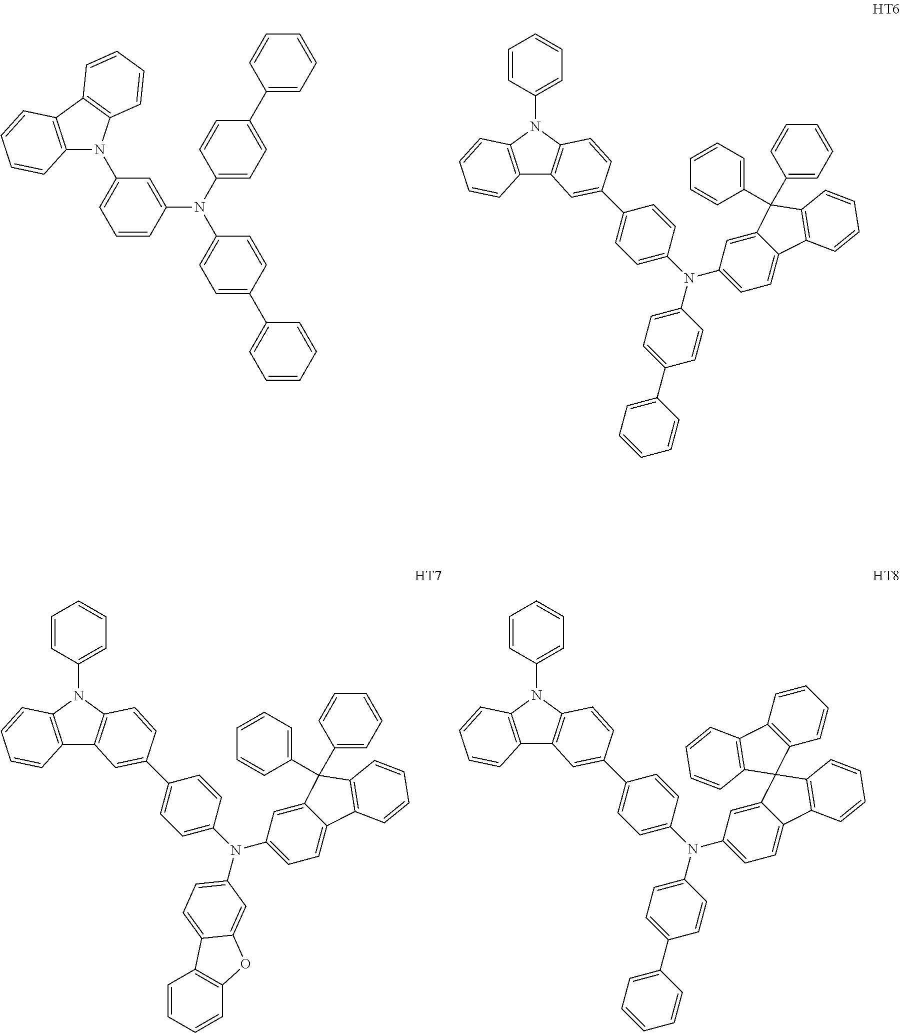 [SMILES: c1ccc(-c2ccc(N(c3ccc(-c4ccccc4)cc3)c3cccc(-n4c5ccccc5c5ccccc54)c3)cc2)cc1 ; c1ccc(-c2ccc(N(c3ccc(-c4ccc5c(c4)c4ccccc4n5-c4ccccc4)cc3)c3ccc4c(c3)C(c3ccccc3)(c3ccccc3)c3ccccc3-4)cc2)cc1 ; c1ccc(-n2c3ccccc3c3cc(-c4ccc(N(c5ccc6c(c5)C(c5ccccc5)(c5ccccc5)c5ccccc5-6)c5ccc6c(c5)oc5ccccc56)cc4)ccc32)cc1 ; c1ccc(-c2ccc(N(c3ccc(-c4ccc5c(c4)c4ccccc4n5-c4ccccc4)cc3)c3ccc4c(c3)C3(c5ccccc5-c5ccccc53)c3ccccc3-4)cc2)cc1]

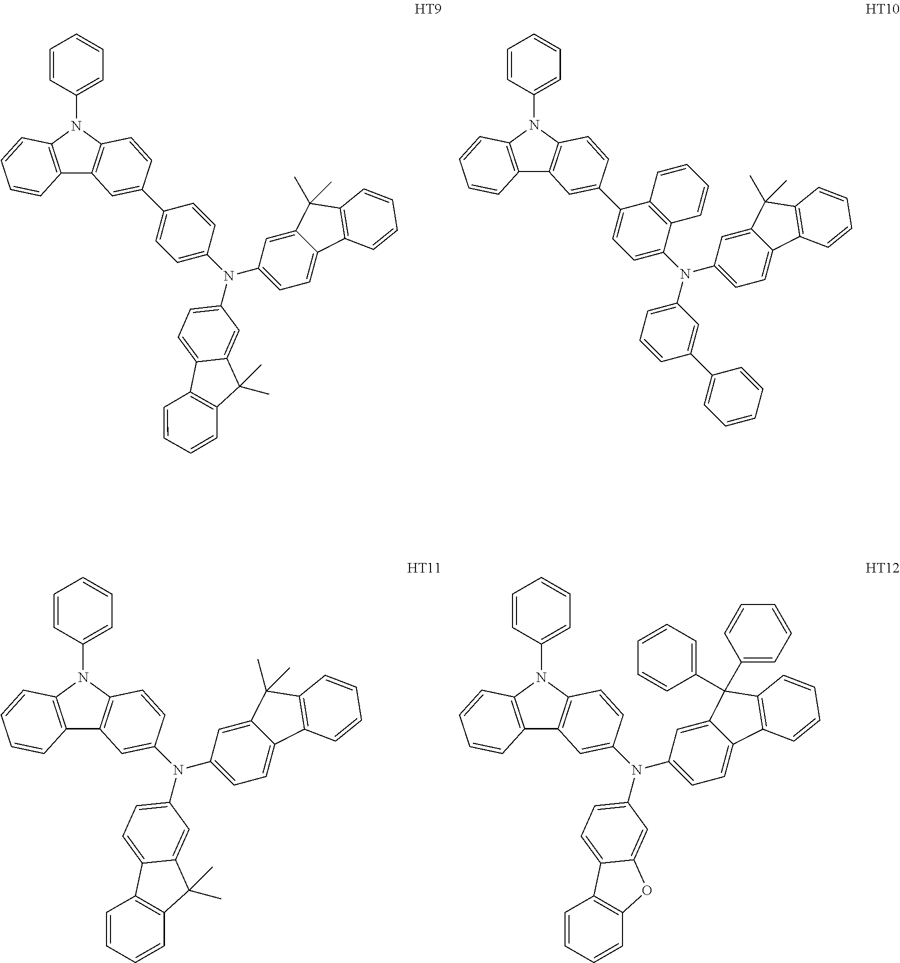 [SMILES: CC1(C)c2ccccc2-c2ccc(N(c3ccc(-c4ccc5c(c4)c4ccccc4n5-c4ccccc4)cc3)c3ccc4c(c3)C(C)(C)c3ccccc3-4)cc21 ; CC1(C)c2ccccc2-c2ccc(N(c3cccc(-c4ccccc4)c3)c3ccc(-c4ccc5c(c4)c4ccccc4n5-c4ccccc4)c4ccccc34)cc21 ; CC1(C)c2ccccc2-c2ccc(N(c3ccc4c(c3)C(C)(C)c3ccccc3-4)c3ccc4c(c3)c3ccccc3n4-c3ccccc3)cc21 ; c1ccc(-n2c3ccccc3c3cc(N(c4ccc5c(c4)C(c4ccccc4)(c4ccccc4)c4ccccc4-5)c4ccc5c(c4)oc4ccccc45)ccc32)cc1]

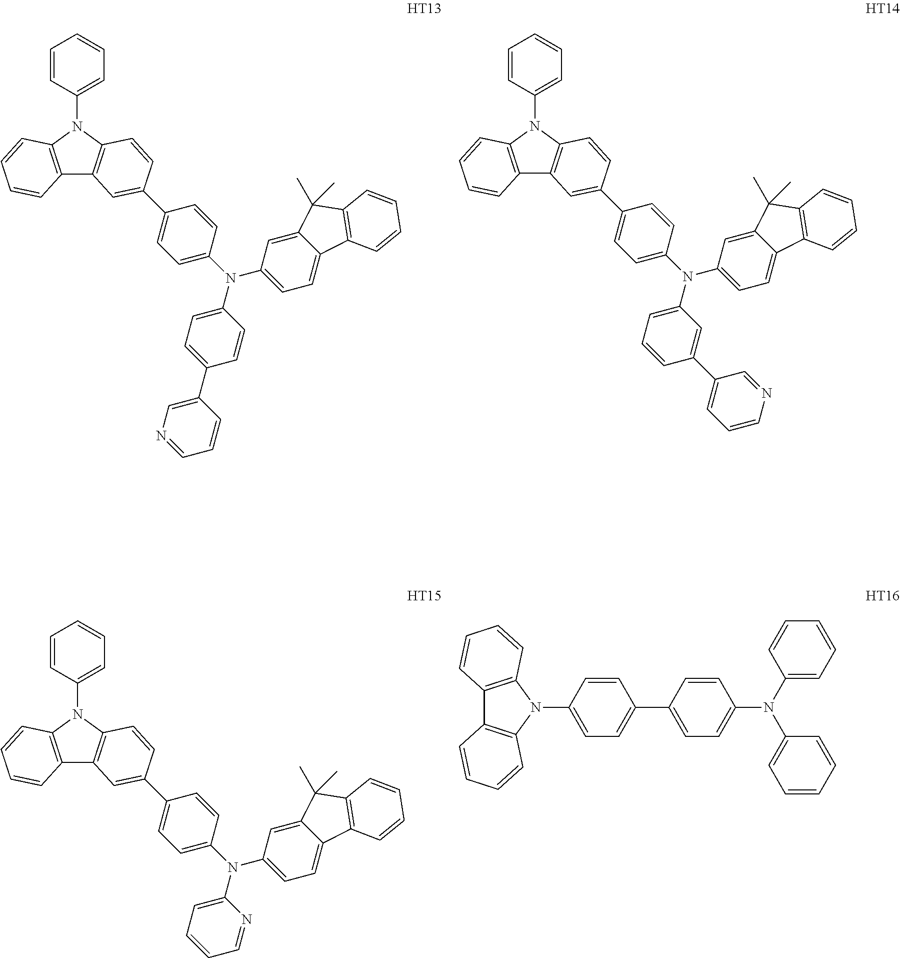 [SMILES: CC1(C)c2ccccc2-c2ccc(N(c3ccc(-c4cccnc4)cc3)c3ccc(-c4ccc5c(c4)c4ccccc4n5-c4ccccc4)cc3)cc21 ; CC1(C)c2ccccc2-c2ccc(N(c3ccc(-c4ccc5c(c4)c4ccccc4n5-c4ccccc4)cc3)c3cccc(-c4cccnc4)c3)cc21 ; CC1(C)c2ccccc2-c2ccc(N(c3ccc(-c4ccc5c(c4)c4ccccc4n5-c4ccccc4)cc3)c3ccccn3)cc21 ; c1ccc(N(c2ccccc2)c2ccc(-c3ccc(-n4c5ccccc5c5ccccc54)cc3)cc2)cc1]

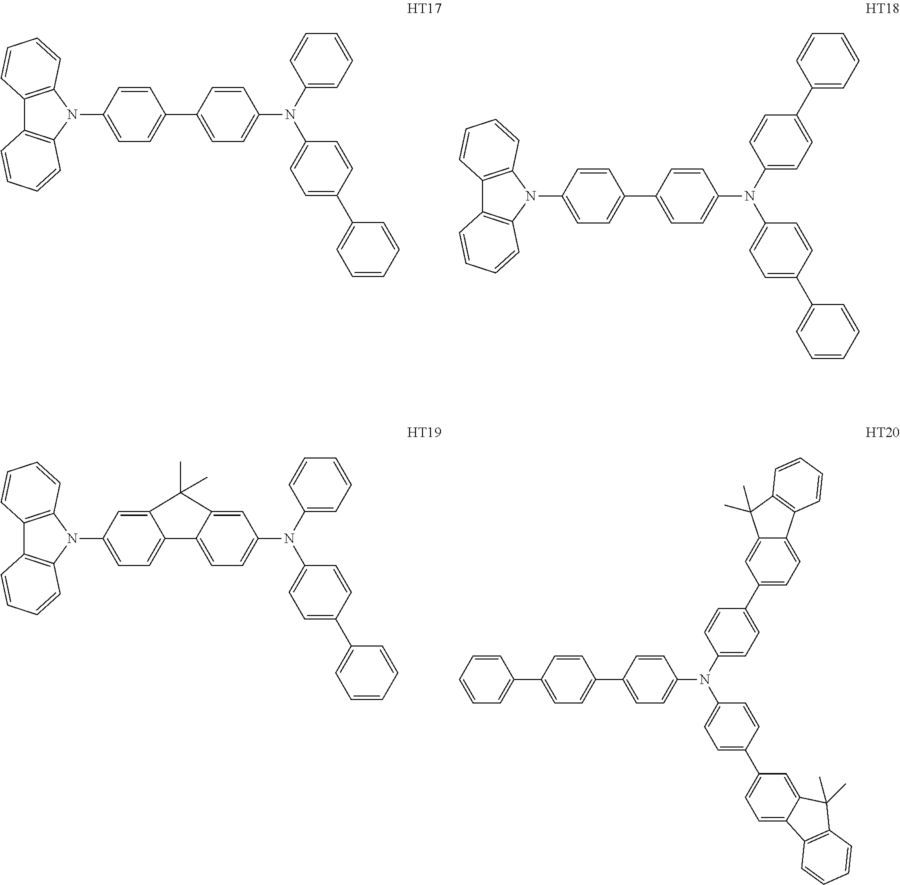 [SMILES: c1ccc(-c2ccc(N(c3ccccc3)c3ccc(-c4ccc(-n5c6ccccc6c6ccccc65)cc4)cc3)cc2)cc1 ; c1ccc(-c2ccc(N(c3ccc(-c4ccccc4)cc3)c3ccc(-c4ccc(-n5c6ccccc6c6ccccc65)cc4)cc3)cc2)cc1 ; CC1(C)c2cc(N(c3ccccc3)c3ccc(-c4ccccc4)cc3)ccc2-c2ccc(-n3c4ccccc4c4ccccc43)cc21 ; CC1(C)c2ccccc2-c2ccc(-c3ccc(N(c4ccc(-c5ccc(-c6ccccc6)cc5)cc4)c4ccc(-c5ccc6c(c5)C(C)(C)c5ccccc5-6)cc4)cc3)cc21]

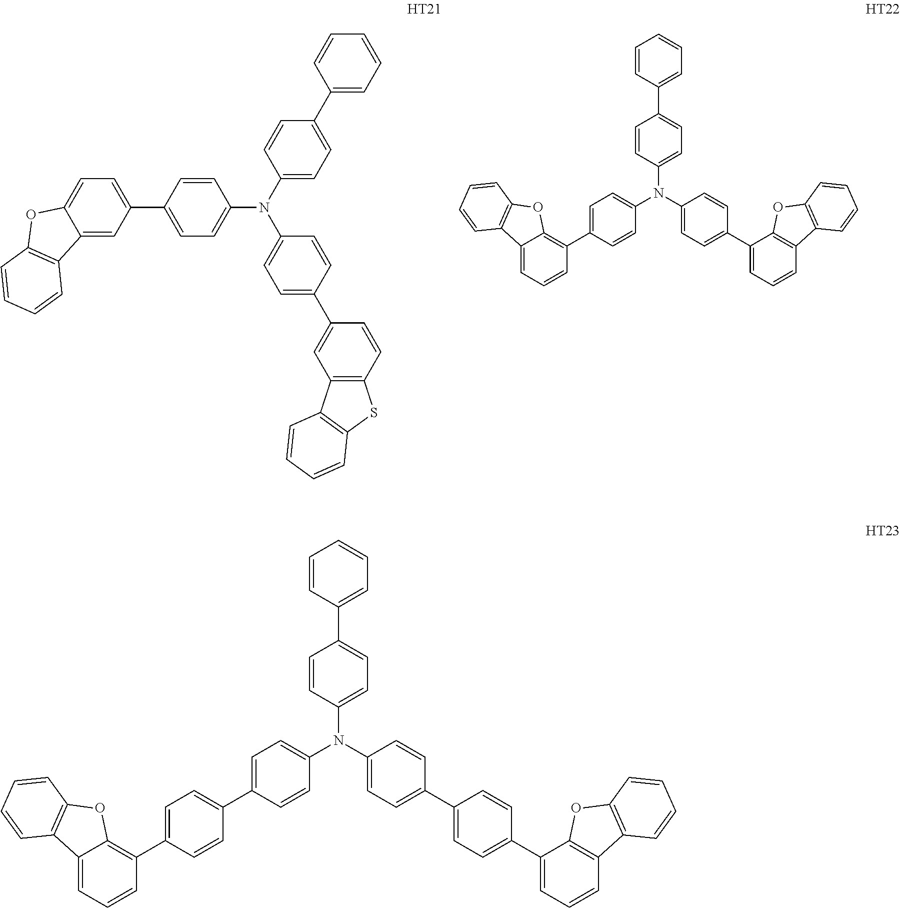 [SMILES: c1ccc(-c2ccc(N(c3ccc(-c4ccc5oc6ccccc6c5c4)cc3)c3ccc(-c4ccc5sc6ccccc6c5c4)cc3)cc2)cc1 ; c1ccc(-c2ccc(N(c3ccc(-c4cccc5c4oc4ccccc45)cc3)c3ccc(-c4cccc5c4oc4ccccc45)cc3)cc2)cc1 ; c1ccc(-c2ccc(N(c3ccc(-c4ccc(-c5cccc6c5oc5ccccc56)cc4)cc3)c3ccc(-c4ccc(-c5cccc6c5oc5ccccc56)cc4)cc3)cc2)cc1]

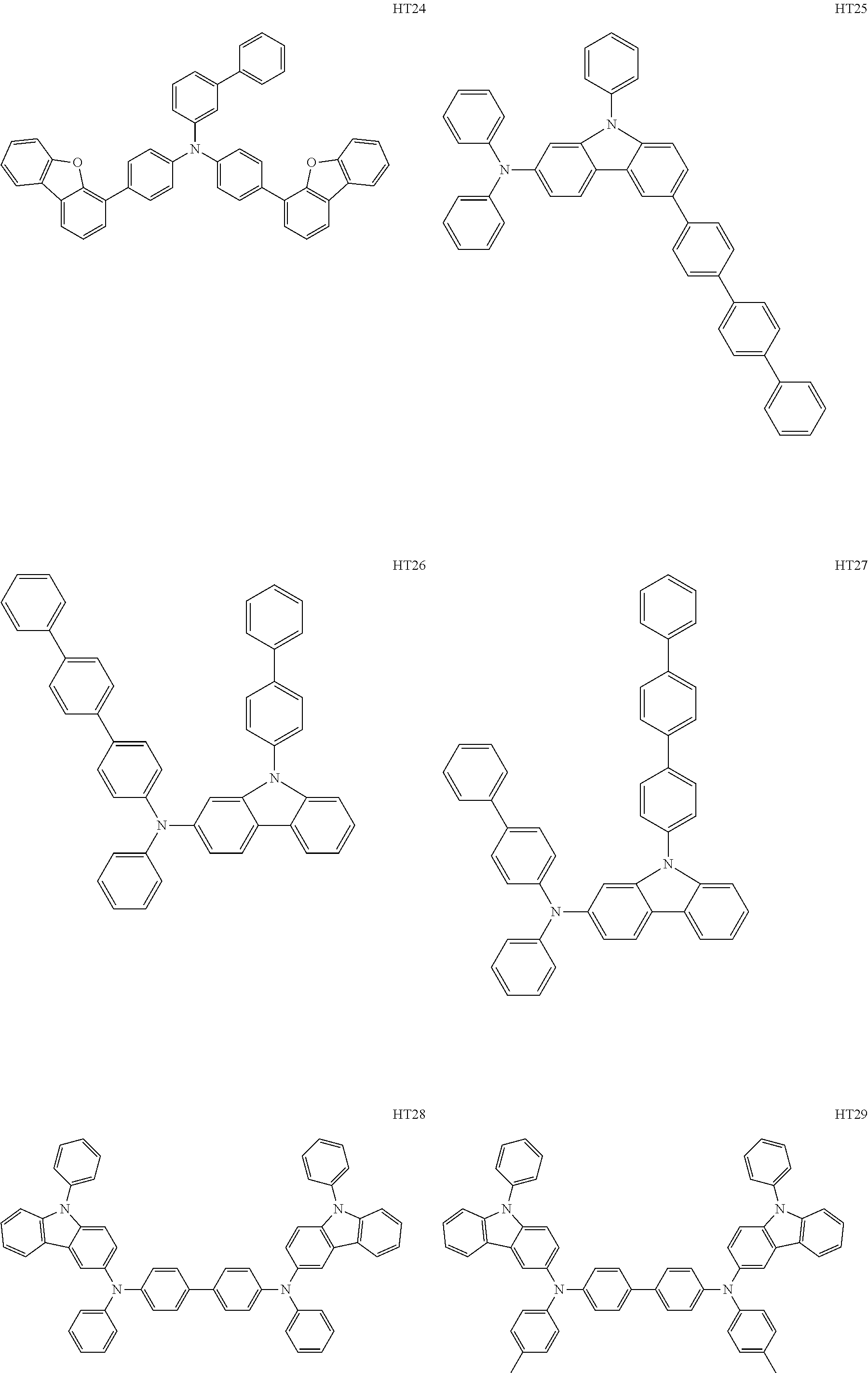

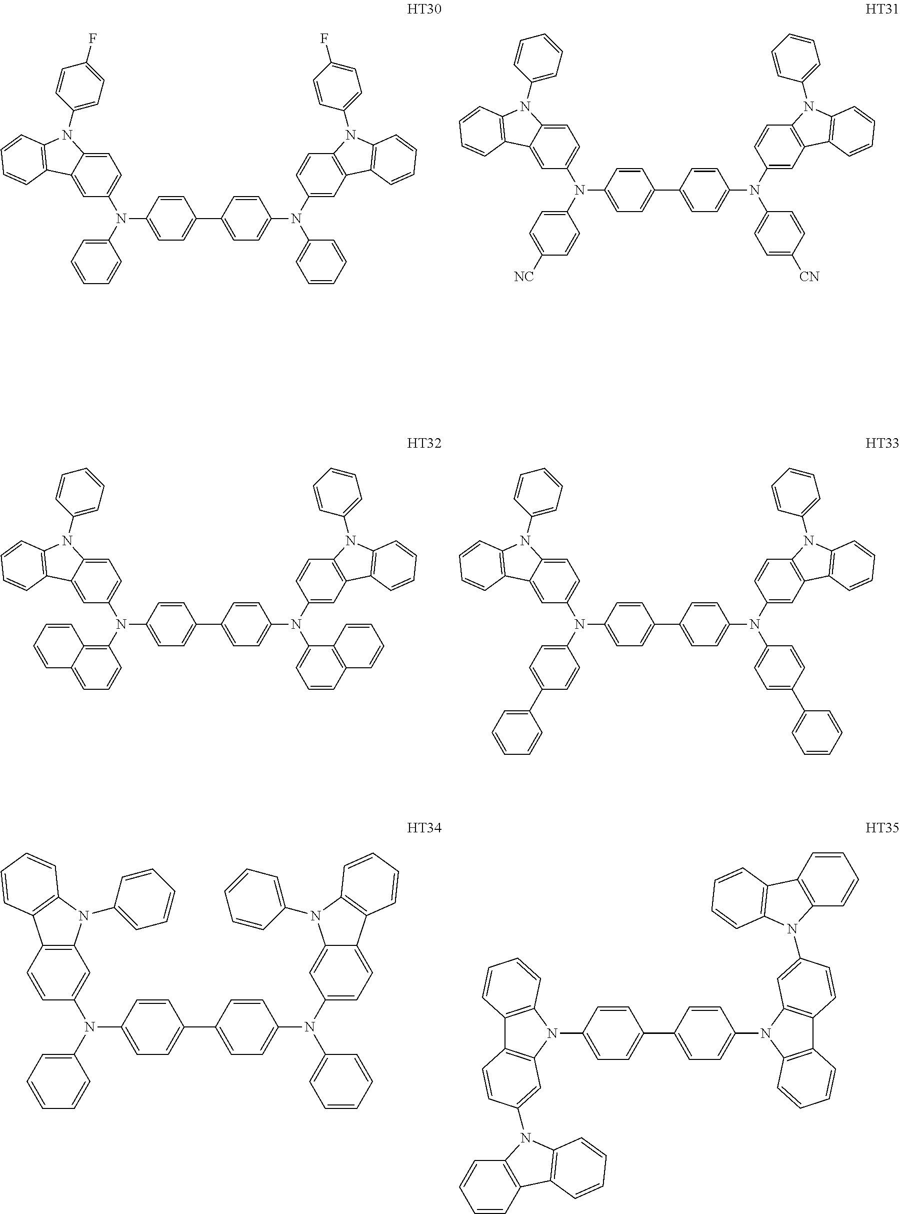

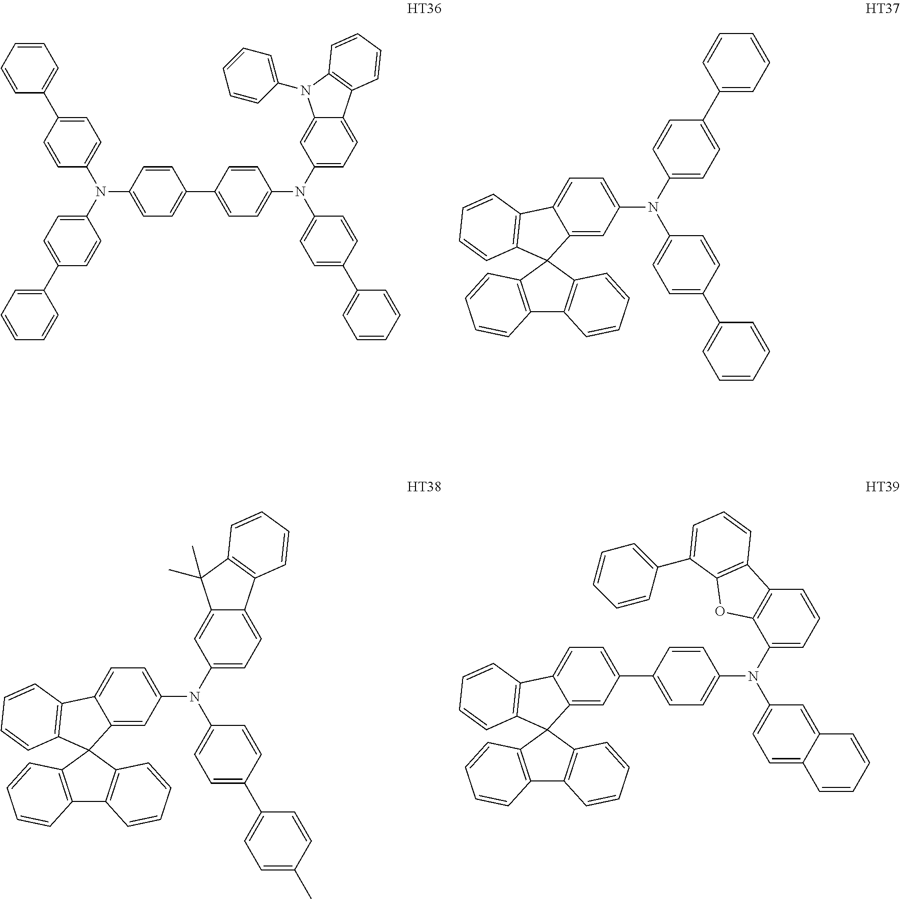 [SMILES: c1ccc(-c2ccc(N(c3ccc(-c4ccccc4)cc3)c3ccc(-c4ccc(N(c5ccc(-c6ccccc6)cc5)c5ccc6c7ccccc7n(-c7ccccc7)c6c5)cc4)cc3)cc2)cc1 ; c1ccc(-c2ccc(N(c3ccc(-c4ccccc4)cc3)c3ccc4c(c3)C3(c5ccccc5-c5ccccc53)c3ccccc3-4)cc2)cc1 ; Cc1ccc(-c2ccc(N(c3ccc4c(c3)C(C)(C)c3ccccc3-4)c3ccc4c(c3)C3(c5ccccc5-c5ccccc53)c3ccccc3-4)cc2)cc1 ; c1ccc(-c2cccc3c2oc2c(N(c4ccc(-c5ccc6c(c5)C5(c7ccccc7-c7ccccc75)c5ccccc5-6)cc4)c4ccc5ccccc5c4)cccc23)cc1]

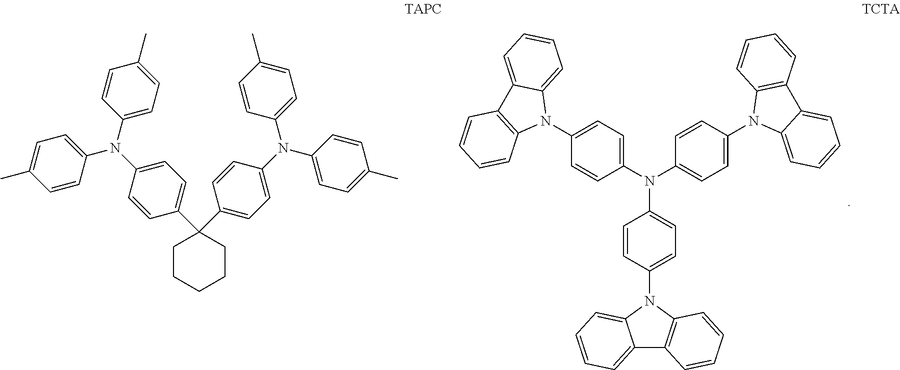

In one or more embodiments, the hole transport region 12 may include at least one of 4,4′-cyclohexylidenebis[N,N-bis(4-methylphenyl)benzenamine] (TAPC), tris(4-carbazoyl-9-ylphenyl)amine (TCTA), or a combination thereof.

In one or more embodiments, hole transport region 12 of the organic light-emitting device 10 may further include a p-dopant. When the hole transport region 12 further includes a p-dopant, the hole transport region 12 may have a matrix (for example, at least one of compounds represented by Formulae 201 to 205) and a p-dopant included in the matrix. The p-dopant may be uniformly or non-uniformly doped in the hole transport region 12.

In an embodiment, the LUMO energy level of the p-dopant may be about −3.5 eV or less.

The p-dopant may include at least one of a quinone derivative, a metal oxide, a cyano group-containing compound, or a combination thereof, but embodiments of the present disclosure are not limited thereto.

In an embodiment, the p-dopant may include at least one of:
a quinone derivative, such as tetracyanoquinodimethane (TCNQ),2,3,5,6-tetrafluoro-7,7,8,8-tetracyanoquinodimethane (F4-TCNQ), and F6-TCNNQ;
a metal oxide, such as tungsten oxide or molybdenum oxide; 1,4,5,8,9,12-hexaazatriphenylene-hexacarbonitrile (HAT-CN);
a compound represented by Formula 221,
or a combination thereof,
but embodiments of the present disclosure are not limited thereto:

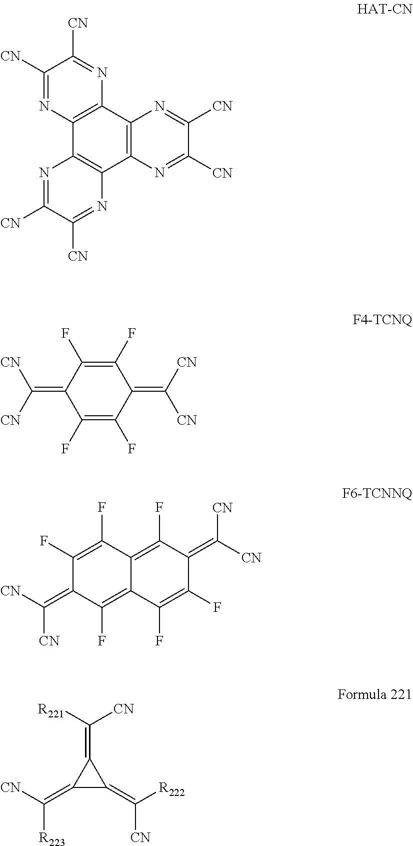

wherein, in Formula 221,
R₂₂₁ to R₂₂₃ may each independently be a substituted or unsubstituted C₃-C₁₀ cycloalkyl group, a substituted or unsubstituted C₁-C₁₀ heterocycloalkyl group, a substituted or unsubstituted C₃-C₁₀ cycloalkenyl group, a substituted or unsubstituted C₂-C₁₀ heterocycloalkenyl group, a substituted or unsubstituted C₆-C₆₀ aryl group, a substituted or unsubstituted C₁-C₆₀ heteroaryl group, a substituted or unsubstituted monovalent aromatic condensed polycyclic group, a substituted or unsubstituted monovalent aromatic condensed heteropolycyclic group, a substituted or unsubstituted monovalent non-aromatic condensed polycyclic group, or a substituted or unsubstituted monovalent non-aromatic condensed heteropolycyclic group, wherein at least one of R₂₂₁ to R₂₂₃ may include at least one substituent of a cyano group, —F, —Cl, —Br, —I, a C₁-C₂₀ alkyl group substituted with —F, a C₁-C₂₀ alkyl group substituted with —Cl, a C₁-C₂₀ alkyl group substituted with —Br, a C₁-C₂₀ alkyl group substituted with —I, or a combination thereof.

The hole transport region 12 may have a thickness of about 100 Å to about 10,000 Å, for example, about 400 Å to about 2,000 Å, and the emission layer 15 may have a thickness of about 100 Å to about 3,000 Å, for example, about 300 Å to about 1,000 Å. When the thickness of each of the hole transport region 12 and the emission layer 15 is within these ranges, satisfactory hole transportation characteristics and/or luminescence characteristics may be obtained without a substantial increase in driving voltage.

Electron Transport Region 17

The electron transport region 17 may be arranged between the emission layer 15 and the second electrode 19 of the organic light-emitting device 10.

The electron transport region 17 may have a single-layer structure or a multi-layer structure.

For example, the electron transport region 17 may have an electron transport layer, an electron transport layer/electron injection layer structure, a buffer layer/electron transport layer structure, hole blocking layer/electron transport layer structure, a buffer layer/electron transport layer/electron injection layer structure, or a hole blocking layer/electron transport layer/electron injection layer structure. The electron transport region 17 may further include an electron control layer.

The electron transport region 17 may include a known electron-transporting material.

The electron transport region 17 (for example, a buffer layer, a hole blocking layer, an electron control layer, or an electron transport layer in the electron transport region) may include a metal-free compound containing at least one π electron-deficient nitrogen-containing cyclic group. The π electron-deficient nitrogen-containing cyclic group is the same as described above.

In an embodiment, the electron transport region may include a compound represented by Formula 601 below:

[Ar₆₀₁]ₓₑ₁₁-[(L₆₀₁)ₓₑ₁-R₆₀₁]ₓₑ₂₁ Formula 601
wherein, in Formula 601,
Ar₆₀₁ and L₆₀₁ may each independently be a substituted or unsubstituted C₅-C₆₀ carbocyclic group or a substituted or unsubstituted C₁-C₆₀ heterocyclic group,
xe11 may be 1, 2, or 3,
xe1 may be an integer from 0 to 5,
R₆₀₁ may be a substituted or unsubstituted C₃-C₁₀ cycloalkyl group, a substituted or unsubstituted C₁-C₁₀ heterocycloalkyl group, a substituted or unsubstituted C₃-C₁₀ cycloalkenyl group, a substituted or unsubstituted C₂-C₁₀ heterocycloalkenyl group, a substituted or unsubstituted C₆-C₆₀ aryl group, a substituted or unsubstituted C₆-C₆₀ aryloxy group, a substituted or unsubstituted C₆-C₆₀ arylthio group, a substituted or unsubstituted heteroaryl group, a substituted or unsubstituted monovalent aromatic condensed polycyclic group, a substituted or unsubstituted monovalent aromatic condensed heteropolycyclic group, a substituted or unsubstituted monovalent non-aromatic condensed polycyclic group, a substituted or unsubstituted monovalent non-aromatic condensed heteropolycyclic group, —Si(Q₆₀₁)(Q₆₀₂)(Q₆₀₃), —C(═O)(Q₆₀₁), —S(═O)₂(Q₆₀₁), or —P(═O)(Q₆₀₁)(Q₆₀₂),
Q₆₀₁ to Q₆₀₃ may each independently be a C₁-C₁₀ alkyl group, a C₁-C₁₀ alkoxy group, a phenyl group, a biphenyl group, a terphenyl group, or a naphthyl group, and
xe21 may be an integer from 1 to 5.

In an embodiment, at least one of Ar₆₀₁(s) in the number of xe11 and R₆₀₁(s) in the number of xe21 may include the π electron-deficient nitrogen-containing cyclic group.

In an embodiment, ring Ar₆₀₁ and L₆₀₁ in Formula 601 may each independently be a benzene group, a naphthalene group, a fluorene group, a spiro-bifluorene group, a benzofluorene group, a dibenzofluorene group, a phenalene group, a phenanthrene group, an anthracene group, a fluoranthene group, a triphenylene group, a pyrene group, a chrysene group, a naphthacene group, a picene group, a perylene group, a pentaphene group, an indenoanthracene group, a dibenzofuran group, a dibenzothiophene group, a carbazole group, an imidazole group, a pyrazole group, a thiazole group, an isothiazole group, an oxazole group, an isoxazole group, a pyridine group, a pyrazine group, a pyrimidine group, a pyridazine group, an indazole group, a purine group, a quinoline group, an isoquinoline group, a benzoquinoline group, a phthalazine group, a naphthyridine group, a quinoxaline group, a quinazoline group, a cinnoline group, a phenanthridine group, an acridine group, a phenanthroline group, a phenazine group, a benzimidazole group, an isobenzothiazole group, a benzoxazole group, an isobenzoxazole group, a triazole group, a tetrazole group, an oxadiazole group, a triazine group, a thiadiazole group, an imidazopyridine group, an imidazopyrimidine group, or an azacarbazole group, each unsubstituted or substituted with deuterium, —F, —Cl, —Br, —I, a hydroxyl group, a cyano group, a nitro group, an amidino group, a hydrazine group, a hydrazone group, a C₁-C₂₀ alkyl group, a C₁-C₂₀ alkoxy group, a phenyl group, a biphenyl group, a terphenyl group, a naphthyl group, —Si(Q₃₁)(Q₃₂)(Q₃₃), —S(═O)₂(Q₃₁), —P(═O)(Q₃₁)(Q₃₂), or a combination thereof, and
Q₃₁ to Q₃₃ may each independently be a C₁-C₁₀ alkyl group, a C₁-C₁₀ alkoxy group, a phenyl group, a biphenyl group, a terphenyl group, or a naphthyl group.

When xe11 in Formula 601 is 2 or greater, two or more of Ar₆₀₁ may be linked to each other via a single bond.

In one or more embodiments, Ar₆₀₁ in Formula 601 may be an anthracene group.

In one or more embodiments, the compound represented by Formula 601 may be represented by Formula 601-1:

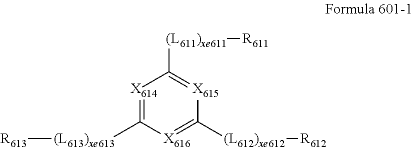

wherein, in Formula 601-1,
X₆₁₄ may be N or C(R₆₁₄), X₆₁₅ may be N or C(R₆₁₅), X₆₁₆ may be N or C(R₆₁₆), and at least one of X₆₁₄ to X₆₁₆ may be N,
L₆₁₁ to L₆₁₃ may each independently be the same as described in connection with L₆₀₁,
xe611 to xe613 may each independently be the same as described in connection with xe1,
R₆₁₁ to R₆₁₃ may each independently be the same as described in connection with R₆₀₁, and
R₆₁₄ to R₆₁₆ may each independently be hydrogen, deuterium, —F, —Cl, —Br, —I, a hydroxyl group, a cyano group, a nitro group, an amidino group, a hydrazino group, a hydrazono group, a C₁-C₂₀ alkyl group, a C₁-C₂₀ alkoxy group, a phenyl group, a biphenyl group, a terphenyl group, or a naphthyl group.

In one or more embodiments, xe1 and xe611 to xe613 in Formulae 601 and 601-1 may each independently be 0, 1, or 2.

In one or more embodiments, R₆₀₁ and R₆₁₁ to R₆₁₃ in Formulae 601 and 601-1 may each independently be a phenyl group, a biphenyl group, a terphenyl group, a naphthyl group, a fluorenyl group, a spiro-bifluorenyl group, a benzofluorenyl group, a dibenzofluorenyl group, a phenanthrenyl group, an anthracenyl group, a fluoranthenyl group, a triphenylenyl group, a pyrenyl group, a chrysenyl group, a perylenyl group, a pentaphenyl group, a hexacenyl group, a pentacenyl group, a thiophenyl group, a furanyl group, a carbazolyl group, an indolyl group, an isoindolyl group, a benzofuranyl group, a benzothiophenyl group, a dibenzofuranyl group, a dibenzothiophenyl group, a benzocarbazolyl group, a dibenzocarbazolyl group, a dibenzosilolyl group, a pyridinyl group, an imidazolyl group, a pyrazolyl group, a thiazolyl group, an isothiazolyl group, an oxazolyl group, an isoxazolyl group, a thiadiazolyl group, an oxadiazolyl group, a pyrazinyl group, a pyrimidinyl group, a pyridazinyl group, a triazinyl group, a quinolinyl group, an isoquinolinyl group, a benzoquinolinyl group, a phthalazinyl group, a naphthyridinyl group, a quinoxalinyl group, a quinazolinyl group, a cinnolinyl group, a phenanthridinyl group, an acridinyl group, a phenanthrolinyl group, a phenazinyl group, a benzimidazolyl group, an isobenzothiazolyl group, a benzoxazolyl group, an isobenzoxazolyl group, a triazolyl group, a tetrazolyl group, an imidazopyridinyl group, an imidazopyrimidinyl group, or an azacarbazolyl group, each unsubstituted or substituted with deuterium, —F, —Cl, —Br, —I, a hydroxyl group, a cyano group, a nitro group, an amidino group, a hydrazine group, a hydrazone group, a C₁-C₂₀ alkyl group, a C₁-C₂₀ alkoxy group, a phenyl group, a biphenyl group, a terphenyl group, a naphthyl group, a fluorenyl group, a spiro-bifluorenyl group, a benzofluorenyl group, a dibenzofluorenyl group, a phenanthrenyl group, an anthracenyl group, a fluoranthenyl group, a triphenylenyl group, a pyrenyl group, a chrysenyl group, a perylenyl group, a pentaphenyl group, a hexacenyl group, a pentacenyl group, a thiophenyl group, a furanyl group, a carbazolyl group, an indolyl group, an isoindolyl group, a benzofuranyl group, a benzothiophenyl group, a dibenzofuranyl group, a dibenzothiophenyl group, a benzocarbazolyl group, a dibenzocarbazolyl group, a dibenzosilolyl group, a pyridinyl group, an imidazolyl group, a pyrazolyl group, a thiazolyl group, an isothiazolyl group, an oxazolyl group, an isoxazolyl group, a thiadiazolyl group, an oxadiazolyl group, a pyrazinyl group, a pyrimidinyl group, a pyridazinyl group, a triazinyl group, a quinolinyl group, an isoquinolinyl group, a benzoquinolinyl group, a phthalazinyl group, a naphthyridinyl group, a quinoxalinyl group, a quinazolinyl group, a cinnolinyl group, a phenanthridinyl group, an acridinyl group, a phenanthrolinyl group, a phenazinyl group, a benzimidazolyl group, an isobenzothiazolyl group, a benzoxazolyl group, an isobenzoxazolyl group, a triazolyl group, a tetrazolyl group, an imidazopyridinyl group, an imidazopyrimidinyl group, an azacarbazolyl group, or a combination thereof; or
—S(═O)₂(Q₆₀₁) or —P(═O)(Q₆₀₁)(Q₆₀₂), and
Q₆₀₁ and Q₆₀₂ may each be the same as described in the present specification.

The electron transport region 17 may include at least one compound of Compounds ET1 to ET36, but embodiments of the present disclosure are not limited thereto:

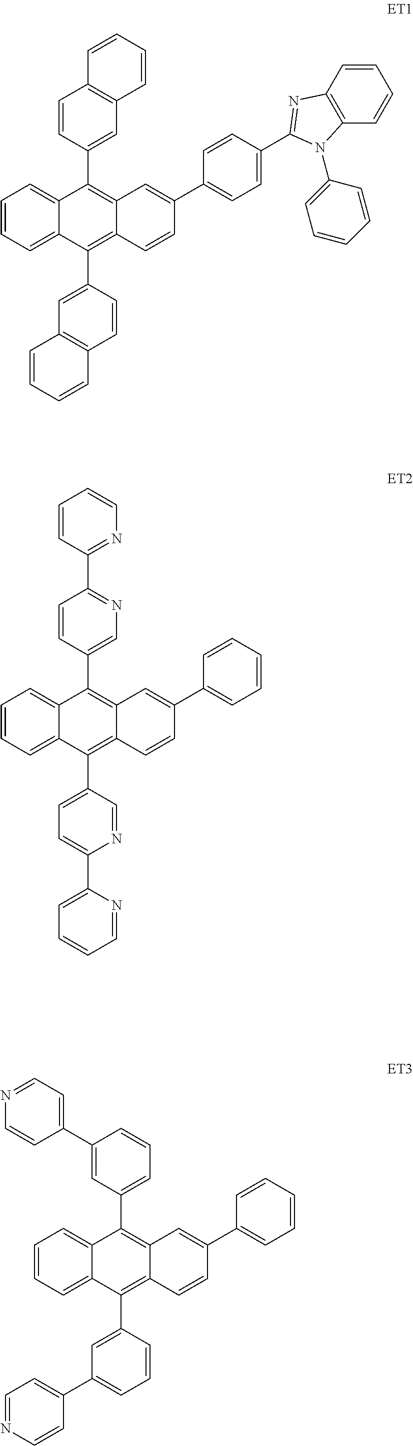 [SMILES: c1ccc(-n2c(-c3ccc(-c4ccc5c(-c6ccc7ccccc7c6)c6ccccc6c(-c6ccc7ccccc7c6)c5c4)cc3)nc3ccccc32)cc1 ; c1ccc(-c2ccc3c(-c4ccc(-c5ccccn5)nc4)c4ccccc4c(-c4ccc(-c5ccccn5)nc4)c3c2)cc1 ; c1ccc(-c2ccc3c(-c4cccc(-c5ccncc5)c4)c4ccccc4c(-c4cccc(-c5ccncc5)c4)c3c2)cc1]

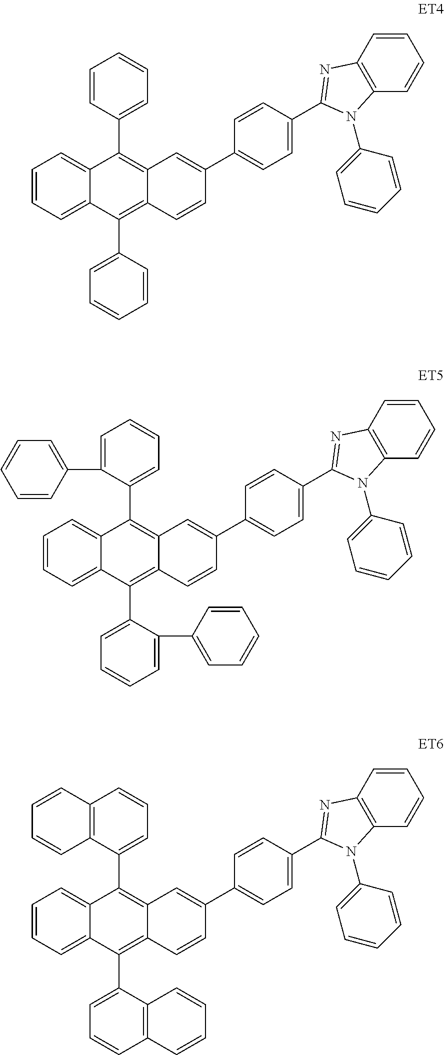 [SMILES: c1ccc(-c2c3ccccc3c(-c3ccccc3)c3cc(-c4ccc(-c5nc6ccccc6n5-c5ccccc5)cc4)ccc23)cc1 ; c1ccc(-c2ccccc2-c2c3ccccc3c(-c3ccccc3-c3ccccc3)c3cc(-c4ccc(-c5nc6ccccc6n5-c5ccccc5)cc4)ccc23)cc1 ; c1ccc(-n2c(-c3ccc(-c4ccc5c(-c6cccc7ccccc67)c6ccccc6c(-c6cccc7ccccc67)c5c4)cc3)nc3ccccc32)cc1]

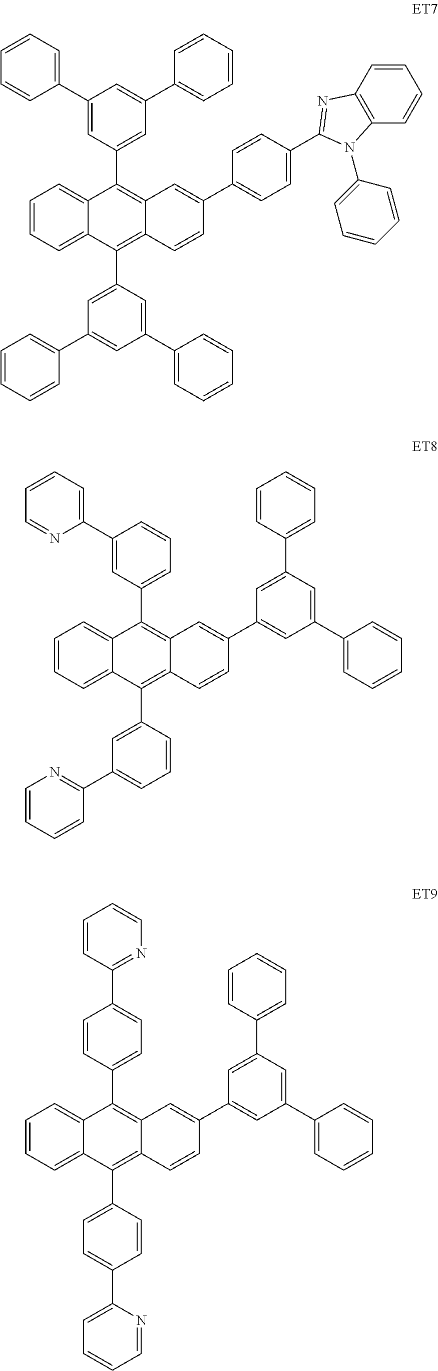 [SMILES: c1ccc(-c2cc(-c3ccccc3)cc(-c3c4ccccc4c(-c4cc(-c5ccccc5)cc(-c5ccccc5)c4)c4cc(-c5ccc(-c6nc7ccccc7n6-c6ccccc6)cc5)ccc34)c2)cc1 ; c1ccc(-c2cc(-c3ccccc3)cc(-c3ccc4c(-c5cccc(-c6ccccn6)c5)c5ccccc5c(-c5cccc(-c6ccccn6)c5)c4c3)c2)cc1 ; c1ccc(-c2cc(-c3ccccc3)cc(-c3ccc4c(-c5ccc(-c6ccccn6)cc5)c5ccccc5c(-c5ccc(-c6ccccn6)cc5)c4c3)c2)cc1]

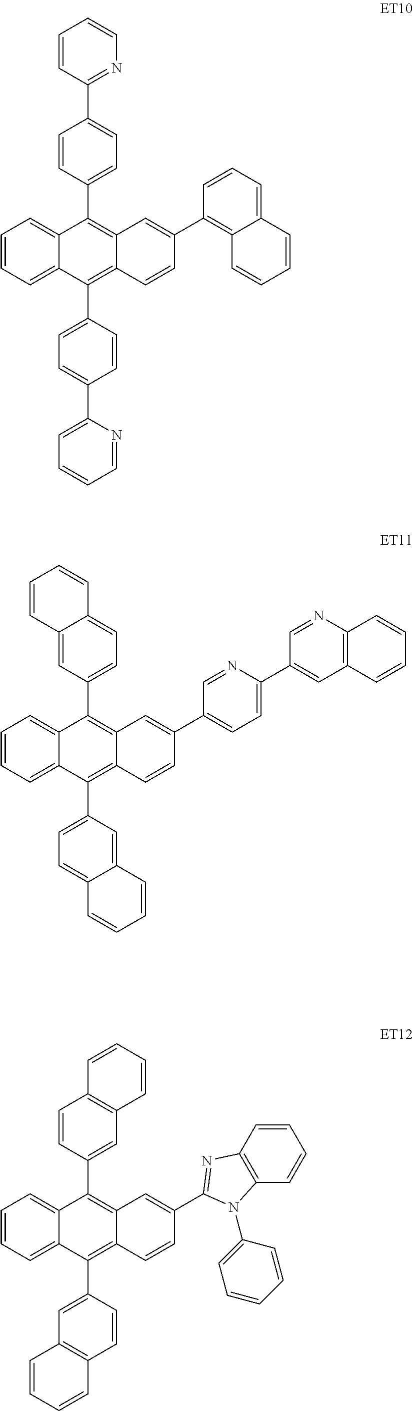 [SMILES: c1ccc(-c2ccc(-c3c4ccccc4c(-c4ccc(-c5ccccn5)cc4)c4cc(-c5cccc6ccccc56)ccc34)cc2)nc1 ; c1ccc2cc(-c3c4ccccc4c(-c4ccc5ccccc5c4)c4cc(-c5ccc(-c6cnc7ccccc7c6)nc5)ccc34)ccc2c1 ; c1ccc(-n2c(-c3ccc4c(-c5ccc6ccccc6c5)c5ccccc5c(-c5ccc6ccccc6c5)c4c3)nc3ccccc32)cc1]

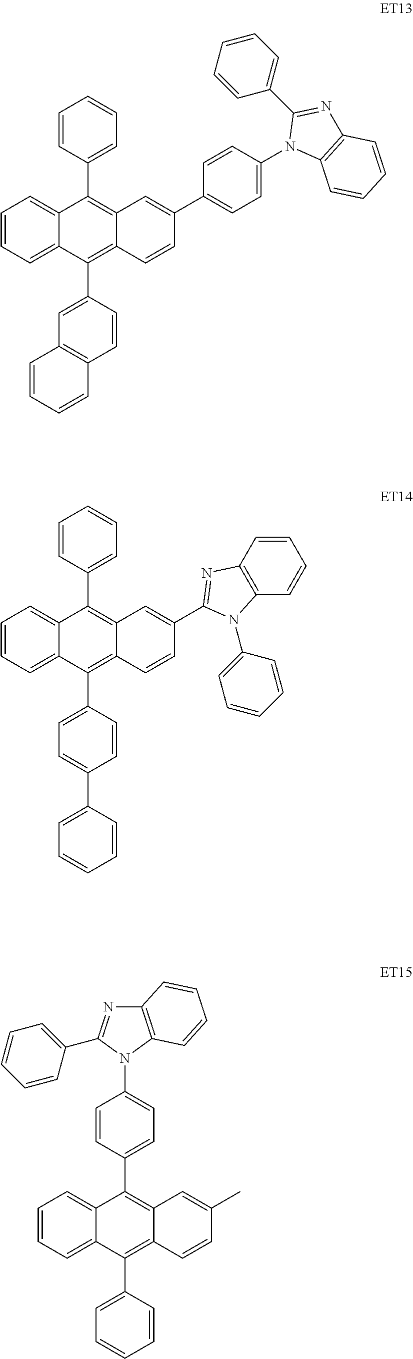 [SMILES: c1ccc(-c2c3ccccc3c(-c3ccc4ccccc4c3)c3ccc(-c4ccc(-n5c(-c6ccccc6)nc6ccccc65)cc4)cc23)cc1 ; c1ccc(-c2ccc(-c3c4ccccc4c(-c4ccccc4)c4cc(-c5nc6ccccc6n5-c5ccccc5)ccc34)cc2)cc1 ; Cc1ccc2c(-c3ccccc3)c3ccccc3c(-c3ccc(-n4c(-c5ccccc5)nc5ccccc54)cc3)c2c1]

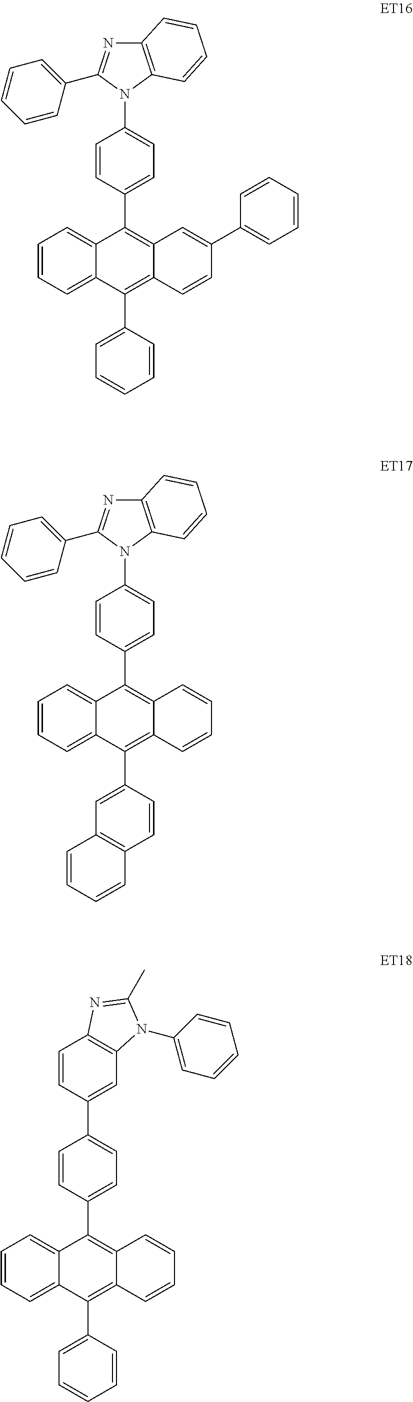 [SMILES: c1ccc(-c2ccc3c(-c4ccccc4)c4ccccc4c(-c4ccc(-n5c(-c6ccccc6)nc6ccccc65)cc4)c3c2)cc1 ; c1ccc(-c2nc3ccccc3n2-c2ccc(-c3c4ccccc4c(-c4ccc5ccccc5c4)c4ccccc34)cc2)cc1 ; Cc1nc2ccc(-c3ccc(-c4c5ccccc5c(-c5ccccc5)c5ccccc45)cc3)cc2n1-c1ccccc1]

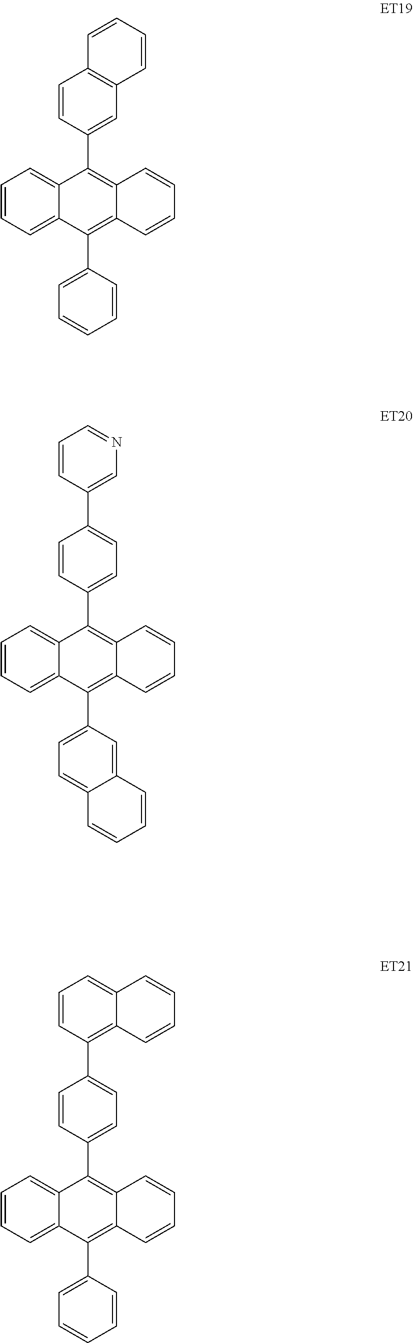

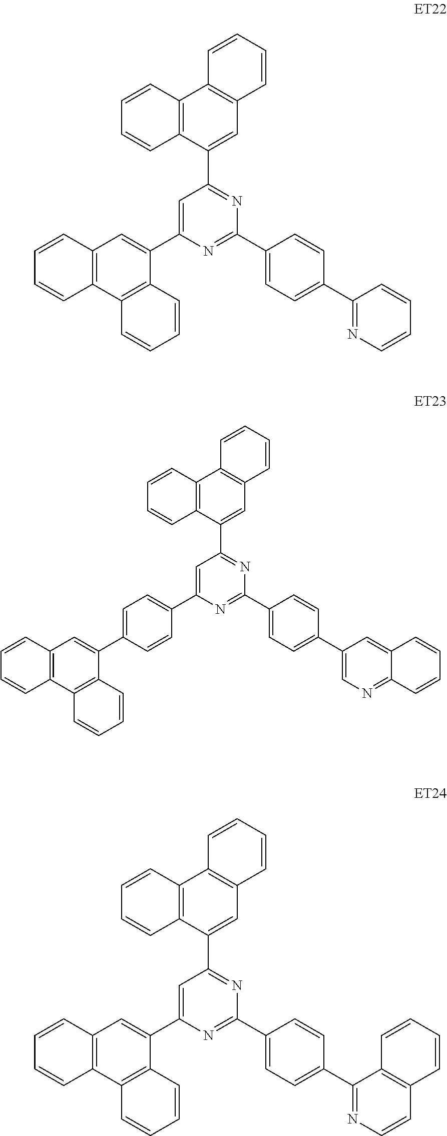 [SMILES: c1ccc(-c2ccc(-c3nc(-c4cc5ccccc5c5ccccc45)cc(-c4cc5ccccc5c5ccccc45)n3)cc2)nc1 ; c1ccc2ncc(-c3ccc(-c4nc(-c5ccc(-c6cc7ccccc7c7ccccc67)cc5)cc(-c5cc6ccccc6c6ccccc56)n4)cc3)cc2c1 ; c1ccc2c(-c3ccc(-c4nc(-c5cc6ccccc6c6ccccc56)cc(-c5cc6ccccc6c6ccccc56)n4)cc3)nccc2c1]

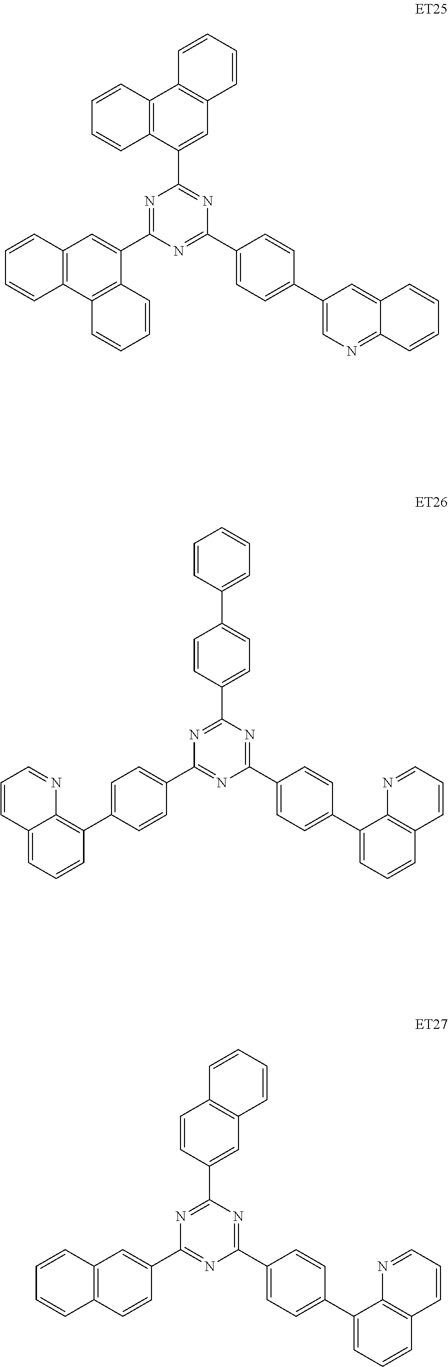 [SMILES: c1ccc2ncc(-c3ccc(-c4nc(-c5cc6ccccc6c6ccccc56)nc(-c5cc6ccccc6c6ccccc56)n4)cc3)cc2c1 ; c1ccc(-c2ccc(-c3nc(-c4ccc(-c5cccc6cccnc56)cc4)nc(-c4ccc(-c5cccc6cccnc56)cc4)n3)cc2)cc1 ; c1ccc2cc(-c3nc(-c4ccc(-c5cccc6cccnc56)cc4)nc(-c4ccc5ccccc5c4)n3)ccc2c1]

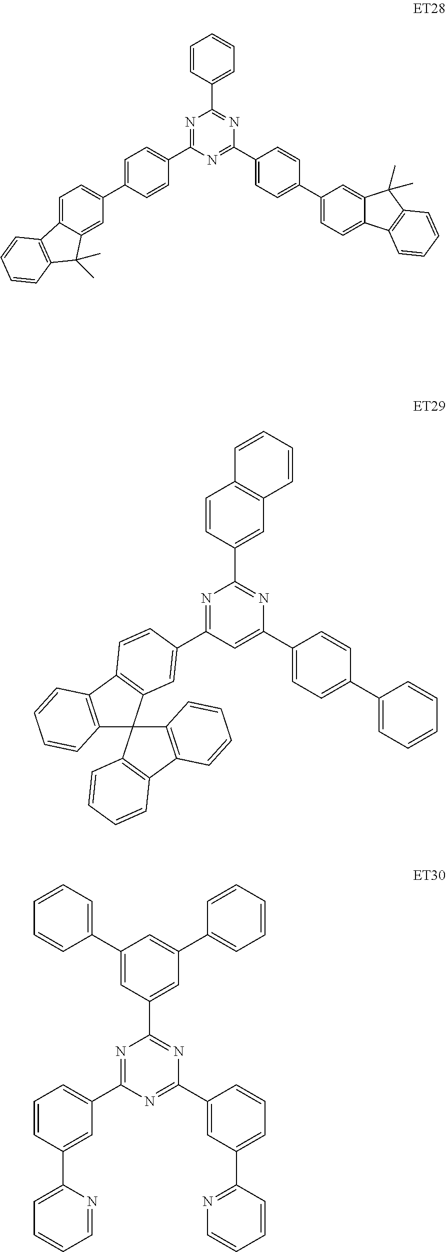 [SMILES: CC1(C)c2ccccc2-c2ccc(-c3ccc(-c4nc(-c5ccccc5)nc(-c5ccc(-c6ccc7c(c6)C(C)(C)c6ccccc6-7)cc5)n4)cc3)cc21 ; c1ccc(-c2ccc(-c3cc(-c4ccc5c(c4)C4(c6ccccc6-c6ccccc64)c4ccccc4-5)nc(-c4ccc5ccccc5c4)n3)cc2)cc1 ; c1ccc(-c2cc(-c3ccccc3)cc(-c3nc(-c4cccc(-c5ccccn5)c4)nc(-c4cccc(-c5ccccn5)c4)n3)c2)cc1]

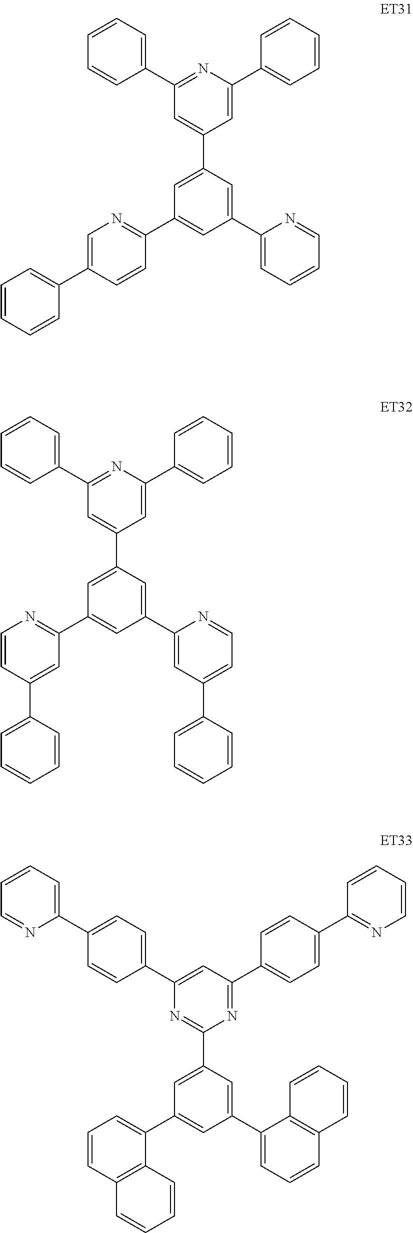 [SMILES: c1ccc(-c2ccc(-c3cc(-c4cc(-c5ccccc5)nc(-c5ccccc5)c4)cc(-c4ccccn4)c3)nc2)cc1 ; c1ccc(-c2ccnc(-c3cc(-c4cc(-c5ccccc5)nc(-c5ccccc5)c4)cc(-c4cc(-c5ccccc5)ccn4)c3)c2)cc1 ; c1ccc(-c2ccc(-c3cc(-c4ccc(-c5ccccn5)cc4)nc(-c4cc(-c5cccc6ccccc56)cc(-c5cccc6ccccc56)c4)n3)cc2)nc1]

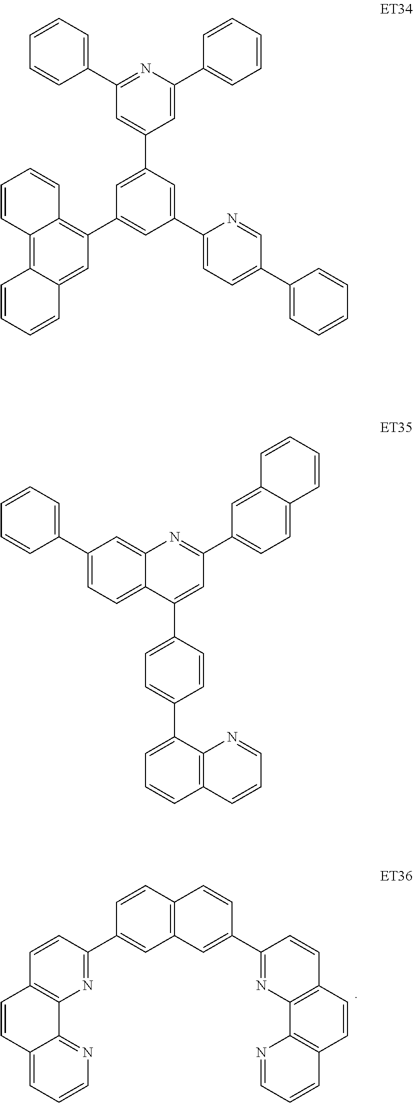 [SMILES: c1ccc(-c2ccc(-c3cc(-c4cc(-c5ccccc5)nc(-c5ccccc5)c4)cc(-c4cc5ccccc5c5ccccc45)c3)nc2)cc1 ; c1ccc(-c2ccc3c(-c4ccc(-c5cccc6cccnc56)cc4)cc(-c4ccc5ccccc5c4)nc3c2)cc1 ; c1cnc2c(c1)ccc1ccc(-c3ccc4ccc(-c5ccc6ccc7cccnc7c6n5)cc4c3)nc12]

In one or more embodiments, the electron transport region 17 may include at least one of 2,9-dimethyl-4,7-diphenyl-1,10-phenanthroline (BCP), 4,7-diphenyl-1,10-phenanthroline (Bphen), Alq3, BAlq, 3-(biphenyl-4-yl)-5-(4-tert-butylphenyl)-4-phenyl-4H-1,2,4-triazole (TAZ), 2,2′,2″-(1,3,5-Benzinetriyl)-tris(1-phenyl-1-H-benzimidazole) (TPBi), NTAZ, or a combination thereof:

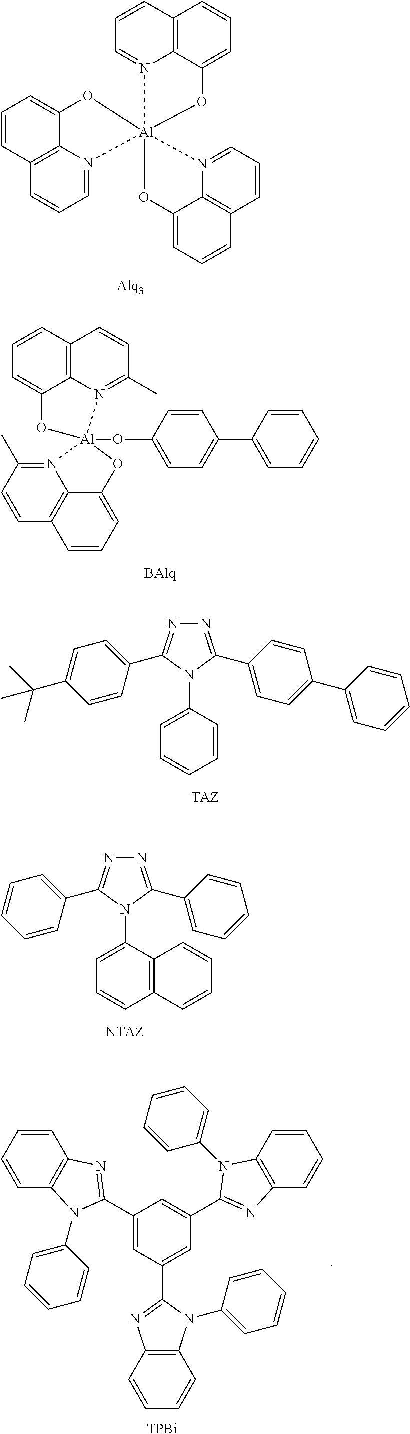

Thicknesses of the buffer layer, the hole blocking layer, and the electron control layer may each independently be in the range of about 20 Å to about 1,000 Å, for example, about 30 Å to about 300 Å. When the thicknesses of the buffer layer, the hole blocking layer, and the electron control layer are within these ranges, excellent hole blocking characteristics or excellent electron control characteristics may be obtained without a substantial increase in driving voltage.

A thickness of the electron transport layer may be in the range of about 100 Å to about 1,000 Å, for example, about 150 Å to about 500 Å. When the thickness of the electron transport layer is within the range described above, the electron transport layer may have satisfactory electron transporting characteristics without a substantial increase in driving voltage.

The electron transport region 17 (for example, the electron transport layer in the electron transport region 17) may further include, in addition to the materials described above, a metal-containing material.

The metal-containing material may include at least one alkali metal complex, alkaline earth-metal complex, or a combination thereof. The alkali metal complex may include a metal ion a L₁ ion, a Na ion, a K ion, a Rb ion, a Cs ion, or a combination thereof, and the alkaline earth-metal complex may include a Be ion, a Mg ion, a Ca ion, a Sr ion, a Ba ion, or a combination thereof. A ligand coordinated with the metal ion of the alkali metal complex or the alkaline earth-metal complex may be a hydroxy quinoline, a hydroxy isoquinoline, a hydroxy benzoquinoline, a hydroxy acridine, a hydroxy phenanthridine, a hydroxy phenyloxazole, a hydroxy phenylthiazole, a hydroxy diphenyloxadiazole, a hydroxy diphenylthiadiazole, a hydroxy phenylpyridine, a hydroxy phenylbenzimidazole, a hydroxy phenylbenzothiazole, a bipyridine, a phenanthroline, or a cyclopentadiene, but embodiments of the present disclosure are not limited thereto.

In an embodiment, the metal-containing material may include a L₁ complex. The Li complex may include, for example, Compound ET-D1 (lithium quinolate, LiQ) or ET-D2:

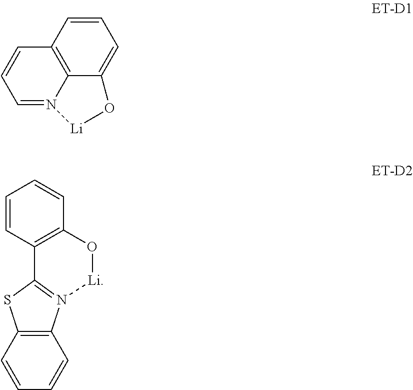

The electron transport region 17 may include an electron injection layer that facilitates the injection of electrons from the second electrode 19. The electron injection layer may directly contact the second electrode 19.

The electron injection layer may have i) a single-layer structure consisting of a single layer including a single material, ii) a single-layer structure consisting of a single layer including multiple materials that are different from each other, or iii) a multi-layer structure consisting of multiple layers including multiple different materials that are different from each other.

The electron injection layer may include an alkali metal, an alkaline earth metal, a rare earth metal, an alkali metal compound, an alkaline earth-metal compound, a rare earth metal compound, an alkali metal complex, an alkaline earth-metal complex, a rare earth metal complex, or any combination thereof.

The alkali metal may be Li, Na, K, Rb, or Cs. In an embodiment, the alkali metal may be Li, Na, or Cs. In one or more embodiments, the alkali metal may be L₁ or Cs, but embodiments of the present disclosure are not limited thereto.

The alkaline earth metal may be Mg, Ca, Sr, or Ba.

The rare earth metal may be Sc, Y, Ce, Tb, Yb, or Gd.

The alkali metal compound, the alkaline earth-metal compound, and the rare earth metal compound may be an oxide or a halide (for example, fluorides, chlorides, bromides, or iodides) of the alkali metal, the alkaline earth-metal, and the rare earth metal.

The alkali metal compound may be an alkali metal oxide, such as Li₂O, Cs₂O, or K₂O, and alkali metal halides, such as LiF, NaF, CsF, KF, LiI, NaI, CsI, or KI. In an embodiment, the alkali metal compound may be LiF, Li₂O, NaF, LiI, NaI, CsI, or KI, but embodiments of the present disclosure are not limited thereto.

The alkaline earth-metal compound may be alkaline earth-metal oxides, such as BaO, SrO, CaO, BaₓSr₁₋ₓO (0<x<1), or BaₓCa₁₋ₓO (0<x<1). In an embodiment, the alkaline earth-metal compound may be BaO, SrO, or CaO, but embodiments of the present disclosure are not limited thereto.

The rare earth metal compound may be YbF₃, ScF₃, ScO₃, Y₂O₃, Ce₂O₃, GdF₃, TbF₃. In an embodiment, the rare earth metal compound may be YbF₃, ScF₃, TbF₃, YbI₃, ScI₃, or TbI₃, but embodiments of the present disclosure are not limited thereto.

The alkali metal complex, the alkaline earth-metal complex, and the rare earth metal complex may include an ion of alkali metal, alkaline earth-metal, or rare earth metal as described above, and a ligand coordinated with a metal ion of the alkali metal complex, the alkaline earth-metal complex, or the rare earth metal complex may be hydroxy quinoline, hydroxy isoquinoline, hydroxy benzoquinoline, hydroxy acridine, hydroxy phenanthridine, hydroxy phenyloxazole, hydroxy phenylthiazole, hydroxy diphenyloxadiazole, hydroxy diphenylthiadiazole, hydroxy phenylpyridine, hydroxy phenylbenzimidazole, hydroxy phenylbenzothiazole, bipyridine, phenanthroline, or cyclopentadiene, but embodiments of the present disclosure are not limited thereto.

The electron injection layer may consist of an alkali metal, an alkaline earth metal, a rare earth metal, an alkali metal compound, an alkaline earth-metal compound, a rare earth metal compound, an alkali metal complex, an alkaline earth-metal complex, a rare earth metal complex, or any combination thereof, as described above. In one or more embodiments, the electron injection layer may further include an organic material. When the electron injection layer further includes an organic material, an alkali metal, an alkaline earth metal, a rare earth metal, an alkali metal compound, an alkaline earth-metal compound, a rare earth metal compound, an alkali metal complex, an alkaline earth-metal complex, a rare earth metal complex, or any combination thereof may be homogeneously or non-homogeneously dispersed in a matrix including the organic material.

A thickness of the electron injection layer may be in a range of about 1 Å to about 100 Å, and, for example, about 3 Å to about 90 Å. When the thickness of the electron injection layer is within the ranges above, satisfactory electron injection characteristics may be obtained without a substantial increase in driving voltage.

Second Electrode 19

The second electrode 19 is arranged on the organic layer 10A having such a structure. The second electrode 19 may be a cathode which is an electron injection electrode, and in this regard, a material for forming the second electrode 19 may be a metal, an alloy, an electrically conductive compound, or a combination thereof, which have a relatively low work function.

The second electrode 19 may include at least one lithium (Li), silver (Ag), magnesium (Mg), aluminum (Al), aluminum-lithium (Al—Li), calcium (Ca), magnesium-indium (Mg—In), magnesium-silver (Mg—Ag), ITO, IZO, or a combination thereof, but embodiments of the present disclosure are not limited thereto. The second electrode 19 may be a transmissive electrode, a semi-transmissive electrode, or a reflective electrode.

The second electrode 19 may have a single-layer structure having a single layer or a multi-layer structure including two or more layers.

Hereinbefore, the organic light-emitting device has been described with reference to FIG. 1, but embodiments of the present disclosure are not limited thereto.

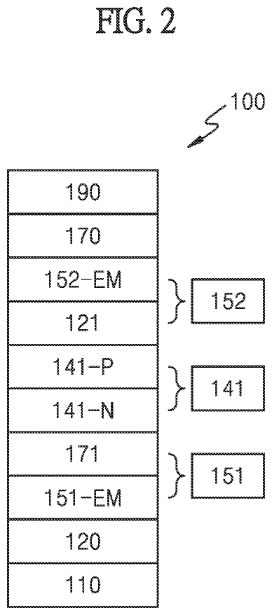

Description of FIG. 2

FIG. 2 is a schematic view of an organic light-emitting device 100 according to an exemplary embodiment.

The organic light-emitting device 100 of FIG. 2 includes a first electrode 110, a second electrode 190 facing the first electrode 110, and a first light-emitting unit 151 and a second light-emitting unit 152 between the first electrode 110 and the second electrode 190. A charge generation layer 141 is arranged between the first emission unit 151 and the second emission unit 152, and the charge generation layer 141 may include an n-type charge generation layer 141-N and a p-type charge generation layer 141-P. The charge generation layer 141 is a layer that generates charge and supplies the charge to neighboring emission units, and any known material may be used therefor.

The first light-emitting unit 151 may include a first emission layer 151-EM, and the second light-emitting unit 152 may include a second emission layer 152-EM. The maximum emission wavelength of light emitted from the first light-emitting unit 151 may be different from the maximum emission wavelength of light emitted from the second light-emitting unit 152. For example, the mixed light including the light emitted from the first light-emitting unit 151 and the light emitted from the second light-emitting unit 152 may be white light, but embodiments of the present disclosure are not limited thereto.

The hole transport region 120 is arranged between the first light-emitting unit 151 and the first electrode 110, and the second light-emitting unit 152 may include the first hole transport region 121 arranged on the side of the first electrode 110.

An electron transport region 170 is arranged between the second light-emitting unit 152 and the second electrode 190, and the first emission unit 151 may include a first electron transport region 171 arranged between the charge generation layer 141 and the first emission layer 151-EM.

The first emission layer 151-EM may include the heterocyclic compound.

The second emission layer 152-EM may include the heterocyclic compound.

The first electrode 110 and the second electrode 190 in FIG. 2 may respectively be the same as described in connection with the first electrode 11 and the second electrode 19 in FIG. 1.

In FIG. 2, the first emission layer 151-EM and the second emission layer 152-EM may each be understood by referring to the description for the emission layer 15 in FIG. 1.

The hole transport region 120 and the first hole transport region 121 in FIG. 2 are each the same as described in connection with the hole transport region 12 in FIG. 1.

The electron transport region 170 and the first electron transport region 171 in FIG. 2 are each the same as described in connection with the electron transport region 17 in FIG. 1.

As described above, referring to FIG. 2, an organic light-emitting device in which each of the first light-emitting unit 151 and the second light-emitting unit 152 includes an emission layer including a host, a dopant, and a sensitizer, has been described. However, the organic light-emitting device may have various other forms. For example, one of the first light-emitting unit 151 and the second light-emitting unit 152 of the organic light-emitting device 100 of FIG. 2 may be replaced with any known light-emitting unit, or may include three or more light-emitting units.

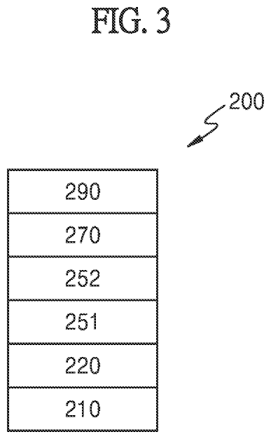

Description of FIG. 3

FIG. 3 is a schematic cross-sectional view of an organic light-emitting device 200 according to another exemplary embodiment.

The organic light-emitting device 200 includes a first electrode 210, a second electrode 290 facing the first electrode 210, and a first emission layer 251 and a second emission layer 252 which are stacked between the first electrode 210 and the second electrode 290.

A maximum emission wavelength of light emitted from the first emission layer 251 may be different from a maximum emission wavelength of light emitted from the second emission layer 252. For example, the mixed light of the light emitted from the first emission layer 251 and the light emitted from the second emission layer 252 may be white light, but embodiments of the present disclosure are not limited thereto.

In one or more embodiments, a hole transport region 220 may be arranged between the first emission layer 251 and the first electrode 210, and an electron transport region 270 may be arranged between the second emission layer 252 and the second electrode 290.

The first emission layer 251 may include the heterocyclic compound.

The second emission layer 252 may include the heterocyclic compound.

The first electrode 210, the hole transporting-region 220, and the second electrode 290 in FIG. 3 are respectively the same as described in connection with the first electrode 11, the hole transport region 12, and the second electrode 19 in FIG. 1.

The first emission layer 251 and the second emission layer 252 in FIG. 3 may each be the same as described in connection with the emission layer 15 in FIG. 1.

The electron transporting-region 270 in FIG. 3 may be the same as described in connection with the electron transport region 17 in FIG. 1.

Hereinabove, referring to FIG. 3, the organic light-emitting device in which both the first emission layer 251 and the second emission layer 252 include the heterocyclic compound is described. However, various modifications of embodiments may be available, and for example, any one of the first emission layer 251 and the second emission layer 252 in FIG. 3 may be replaced by a known layer, three or more emission layers may be included, or an interlayer may be additionally arranged between neighboring emission layers.

Electronic Apparatus

The organic light-emitting device may be included in various electronic apparatuses.

The electronic apparatus may further include a thin-film transistor in addition to the organic light-emitting device as described above. The thin-film transistor may include a source electrode, a drain electrode, and an activation layer, wherein any one of the source electrode and the drain electrode may be electrically connected to any one of the first electrode and the second electrode of the organic light-emitting device.

### DEFINITION OF TERMS

The term “C₁-C₆₀ alkyl group” as used herein refers to a linear or branched saturated aliphatic hydrocarbon monovalent group having 1 to 60 carbon atoms, and examples thereof include a methyl group, an ethyl group, a propyl group, an isobutyl group, a sec-butyl group, a tert-butyl group, a pentyl group, an isoamyl group, and a hexyl group. The term “C₁-C₆₀ alkylene group” as used herein refers to a divalent group having the same structure as the C₁-C₆₀ alkyl group.

The term “C₁-C₆₀ alkoxy group” used herein refers to a monovalent group represented by —OA₁₀₁ (wherein A₁₀₁ is the C₁-C₆₀ alkyl group), and examples thereof include a methoxy group, an ethoxy group, and an isopropyloxy group.

The term “C₂-C₆₀ alkenyl group” as used herein refers to a hydrocarbon group formed by substituting at least one carbon-carbon double bond in the middle or at the terminus of the C₂-C₆₀ alkyl group, and examples thereof include an ethenyl group, a propenyl group, and a butenyl group. The term “C₂-C₆₀ alkenylene group” as used herein refers to a divalent group having the same structure as the C₂-C₆₀ alkenyl group.

The term “C₂-C₆₀ alkynyl group” as used herein refers to a hydrocarbon group formed by substituting at least one carbon-carbon triple bond in the middle or at the terminus of the C₂-C₆₀ alkyl group, and examples thereof include an ethynyl group, and a propynyl group. The term “C₂-C₆₀ alkynylene group” as used herein refers to a divalent group having the same structure as the C₂-C₆₀ alkynyl group.

The term “C₃-C₁₀ cycloalkyl group” as used herein refers to a monovalent saturated hydrocarbon monocyclic group having 3 to 10 carbon atoms, and examples thereof include a cyclopropyl group, a cyclobutyl group, a cyclopentyl group, a cyclohexyl group, and a cycloheptyl group. The term “C₃-C₁₀ cycloalkylene group” as used herein refers to a divalent group having the same structure as the C₃-C₁₀ cycloalkyl group.

The term “C₁-C₁₀ heterocycloalkyl group” as used herein refers to a monovalent saturated monocyclic group having at least one heteroatom N, O, P, Si, and S as a ring-forming atom and 1 to 10 carbon atoms, and non-limiting examples thereof include a tetrahydrofuranyl group and a tetrahydrothiophenyl group. The term “C₁-C₁₀ heterocycloalkylene group” as used herein refers to a divalent group having the same structure as the C₁-C₁₀ heterocycloalkyl group.

The term “C₃-C₁₀ cycloalkenyl group” as used herein refers to a monovalent monocyclic group that has 3 to 10 carbon atoms and at least one carbon-carbon double bond in the ring thereof and no aromaticity, and non-limiting examples thereof include a cyclopentenyl group, a cyclohexenyl group, and a cycloheptenyl group. The term “C₃-C₁₀ cycloalkenylene group” as used herein refers to a divalent group having the same structure as the C₃-C₁₀ cycloalkenyl group.

The term “C₂-C₁₀ heterocycloalkenyl group” as used herein refers to a monovalent monocyclic group that has at least one heteroatom N, O, P, Si, S, B, Ge, Te, or a combination thereof as a ring-forming atom, 2 to 10 carbon atoms, and at least one carbon-carbon double bond in its ring. Examples of the C₂-C₁₀ heterocycloalkenyl group are a 2,3-dihydrofuranyl group, and a 2,3-dihydrothiophenyl group. The term “C₂-C₁₀ heterocycloalkenylene group” as used herein refers to a divalent group having the same structure as the C₂-C₁₀ heterocycloalkenyl group.

The term “C₆-C₆₀ aryl group” as used herein refers to a monovalent group having a carbocyclic aromatic system having 6 to 60 carbon atoms, and the term “C₆-C₆₀ arylene group” as used herein refers to a divalent group having a carbocyclic aromatic system having 6 to 60 carbon atoms. Examples of the C₆-C₆₀ aryl group include a phenyl group, a naphthyl group, an anthracenyl group, a phenanthrenyl group, a pyrenyl group, and a chrysenyl group. When the C₆-C₆₀ aryl group and the C₆-C₆₀ arylene group each include two or more rings, the rings may be fused to each other.

The term “C₁-C₆₀ heteroaryl group” as used herein refers to a monovalent group having a heterocyclic aromatic system that has at least one heteroatom N, O, P, Si, Si, S, B, Ge, Te, or a combination thereof as a ring-forming atom, and 1 to 60 carbon atoms. The term “C₁-C₆₀ heteroarylene group” as used herein refers to a divalent group having a carbocyclic aromatic system that has at least one heteroatom N, O, P, Si, S, B, Ge, Te, or a combination thereof as a ring-forming atom, and 1 to 60 carbon atoms. Examples of the C₁-C₆₀ heteroaryl group include a pyridinyl group, a pyrimidinyl group, a pyrazinyl group, a pyridazinyl group, a triazinyl group, a quinolinyl group, and an isoquinolinyl group. When the C₆-C₆₀ heteroaryl group and the C₆-C₆₀ heteroarylene group each include two or more rings, the rings may be fused to each other.

The term “C₆-C₆₀ aryloxy group” as used herein indicates —OA₁₀₂ (wherein A₁₀₂ is the C₆-C₆₀ aryl group), and the term “C₆-C₆₀ arylthio group” as used herein indicates —SA₁₀₃ (wherein A₁₀₃ is the C₆-C₆₀ aryl group).

The term “monovalent aromatic condensed polycyclic group” as used herein refers to a monovalent group having two or more rings condensed and only carbon atoms (for example, the number of carbon atoms may be in a range of 8 to 60) as ring-forming atoms, wherein the molecular structure as a whole is aromatic. The term “divalent aromatic condensed polycyclic group” as used herein refers to a divalent group having the same structure as the monovalent aromatic condensed polycyclic group described above.

The term “monovalent aromatic condensed heteropolycyclic group” as used herein refers to a monovalent group having at least two rings condensed and a heteroatom N, O, P, Si, Si, S, B, Ge, Te, or a combination thereof as well as carbon atoms (for example, the number of carbon atoms may be in a range of 1 to 60) as ring-forming atoms, wherein the molecular structure as a whole is aromatic. The term “divalent aromatic condensed heteropolycyclic group” as used herein refers to a divalent group having the same structure as the monovalent aromatic condensed heteropolycyclic group described above.

The term “monovalent non-aromatic condensed polycyclic group” used herein refers to a monovalent group in which two or more rings are condensed with each other, only carbon is used as a ring-forming atom (for example, the number of carbon atoms may be 8 to 60) and the whole molecule is a non-aromaticity group. Examples of the monovalent non-aromatic condensed polycyclic group include a fluorenyl group. The term “divalent non-aromatic condensed polycyclic group” as used herein refers to a divalent group having the same structure as the monovalent non-aromatic condensed polycyclic group described above.

The term “monovalent non-aromatic condensed heteropolycyclic group” as used herein refers to a monovalent group having two or more rings condensed to each other, a heteroatom N, O, P, Si, Si, S, B, Ge, Te, or a combination thereof, other than carbon atoms(for example, having 1 to 60 carbon atoms), as a ring-forming atom, and no aromaticity in its entire molecular structure. Examples of the monovalent non-aromatic condensed heteropolycyclic group include a carbazolyl group. The term “divalent non-aromatic condensed heteropolycyclic group” as used herein refers to a divalent group having the same structure as the monovalent non-aromatic condensed heteropolycyclic group described above.

The term “π electron-depleted nitrogen-containing C₁-C₆₀ cyclic group” as used herein refers to a cyclic group having 1 to 60 carbon atoms and including at least one *—N=*′ (wherein * and *′ each indicate a binding site to an adjacent atom) as a ring-forming moiety. For example, the π electron-depleted nitrogen-containing C₁-C₆₀ cyclic group may be a) a first ring, b) a condensed ring in which at least two first rings are condensed, or c) a condensed ring in which at least one first ring and at least one second ring are condensed.

The term “π electron-rich C₃-C₆₀ cyclic group” as used herein refers to a cyclic group having 3 to 60 carbon atoms and not including at least one *—N=*′ (wherein * and *′ each indicate a binding site to an adjacent atom) as a ring-forming moiety. For example, the π electron-rich C₃-C₆₀ cyclic group may be a) a second ring or b) a condensed ring in which at least two second rings are condensed.

The term “C₅-C₆₀ carbocyclic group” as used herein refers to a monocyclic or polycyclic group having 5 to 60 carbon atoms, and may be, for example, a) a third ring or b) a condensed ring in which two or more third rings are condensed with each other.

The term “C₁-C₆₀ heterocyclic group” as used herein refers to a monocyclic or polycyclic group that has 1 to 60 carbon atoms and includes at least one heteroatom, and may be, for example, a) a fourth ring, b) a condensed ring in which two or more fourth rings are condensed with each other, or c) a condensed ring in which at least one third ring is condensed with at least one fourth ring.

The “first ring” as used herein may be an imidazole group, a pyrazole group, a thiazole group, an isothiazole group, an oxazole group, an isoxazole group, a pyridine group, a pyrazine group, a pyridazine group, a pyrimidine group, a triazole group, a tetrazole group, an oxadiazole group, a triazine group, or a thiadiazole group.

The “second ring” as used herein may be a benzene group, a cyclopentadiene group, a pyrrole group, a furan group, a thiophene group, or a silole group.

The “third ring” as used herein may be a cyclopentane group, a cyclopentadiene group, an indene group, an adamantane group, a norbornene group, a bicyclo[1.1.1]pentane group, a bicyclo[2.1.1]hexane group, a bicyclo[2.2.1]heptane group (a norbornane group), a bicyclo[2.2.2]octane group, a cyclohexane group, a cyclohexene group, or a benzene group.

The “fourth ring” as used herein may be a furan group, a thiophene group, a pyrrole group, a silole group, an oxazole group, an isoxazole group, an oxadiazole group, an isoxadiazole group, an oxatriazole group, an isoxatriazole group, a thiazole group, an isothiazole group, a thiadiazole group, an isothiadiazole group, a thiatriazole group, an isothiatriazole group, a pyrazole group, an imidazole group, a triazole group, a tetrazole group, an azasilole group, a diazasilole group, a triazasilole group, a pyridine group, a pyrimidine group, a pyrazine group, a pyridazine group, or a triazine group.

In one or more embodiments, the π electron-depleted nitrogen-containing C₁-C₆₀ cyclic group may be an imidazole group, a pyrazole group, a thiazole group, an isothiazole group, an oxazole group, an isoxazole group, a pyridine group, a pyrazine group, a pyridazine group, a pyrimidine group, an indazole group, a purine group, a quinoline group, an isoquinoline group, a benzoquinoline group, a benzoisoquinoline group, a phthalazine group, a naphthyridine group, a quinoxaline group, a benzoquinoxaline group, a quinazoline group, a cinnoline group, a phenanthridine group, an acridine group, a phenanthroline group, a phenazine group, a benzimidazole group, an isobenzothiazole group, a benzoxazole group, an isobenzoxazole group, a triazole group, a tetrazole group, an oxadiazole group, a triazine group, a thiadiazole group, an imidazopyridine group, an imidazopyrimidine group, an azacarbazole group, an azadibenzofuran group, an azadibenzothiophene group, an azadibenzosilole group, an acridine group, or a pyridopyrazine group.

In one or more embodiments, the π electron-rich C₃-C₆₀ cyclic group may be a benzene group, a heptalene group, an indene group, a naphthalene group, an azulene group, an indacene group, an acenaphthylene group, a fluorene group, a spiro-bifluorene group, a benzofluorene group, a dibenzofluorene group, a phenalene group, a phenanthrene group, an anthracene group, a fluoranthene group, a triphenylene group, a pyrene group, a chrysene group, a naphthacene group, a picene group, a perylene group, a pentacene group, a hexacene group, a pentaphene group, a rubicene group, a coronene group, an ovalene group, a pyrrole group, a furan group, a thiophene group, an isoindole group, an indole group, an indene group, a benzofuran group, a benzothiophene group, a benzosilole group, a naphthopyrrole group, a naphthofuran group, a naphthothiophene group, a naphthosilole group, a benzocarbazole group, a dibenzocarbazole group, a dibenzofuran group, a dibenzothiophene group, a carbazole group, a dibenzosilole group, an indenocarbazole group, an indolocarbazole group, a benzofurocarbazole group, a benzothienocarbazole group, a benzosilolocarbazole group, a triindolobenzene group, a pyrrolophenanthrene group, a furanophenanthrene group, a thienophenanthrene group, a benzonaphthofuran group, a benzonapthothiophene group, an (indolo)phenanthrene group, a (benzofurano)phenanthrene group, or a (benzothieno)phenanthrene group.

For example, the C₅-C₆₀ carbocyclic group may be a cyclopentane group, a cyclohexane group, a cyclohexene group, a benzene group, a naphthalene group, an anthracene group, a phenanthrene group, a triphenylene group, a pyrene group, a chrysene group, a 1,2,3,4-tetrahydronaphthalene group, cyclopentadiene group, an indene group, a fluorene group, a 5,6,7,8-tetrahydroisoquinoline group, a 5,6,7,8-tetrahydroquinoline group, an adamantane group, a norbornane group, or a norbornene group.

For example, the C₁-C₆₀ heterocyclic group may be a thiophene group, a furan group, a pyrrole group, a cyclopentadiene group, a silole group, a borole group, a phosphole group, a selenophene group, a germole group, a benzothiophene group, a benzofuran group, an indole group, an indene group, a benzosilole group, a benzoborole group, a benzophosphole group, a benzoselenophene group, a benzogermole group, a dibenzothiophene group, a dibenzofuran group, a carbazole group, a dibenzosilole group, a dibenzoborole group, a dibenzophosphole group, a dibenzoselenophene group, a dibenzogermole group, a dibenzothiophene 5-oxide group, a 9H-fluorene-9-one group, a dibenzothiophene 5,5-dioxide group, an azabenzothiophene group, an azabenzofuran group, an azaindole group, an azaindene group, an azabenzosilole group, an azabenzoborole group, an azabenzophosphole group, an azabenzoselenophene group, an azabenzogermole group, an azadibenzothiophene group, an azadibenzofuran group, an azacarbazole group, an azafluorene group, an azadibenzosilole group, an azadibenzoborole group, an azadibenzophosphole group, an azadibenzoselenophene group, an azadibenzogermole group, an azadibenzothiophene 5-oxide group, an aza-9H-fluorene-9-one group, an azadibenzothiophene 5,5-dioxide group, a pyridine group, a pyrimidine group, a pyrazine group, a pyridazine group, a triazine group, a quinoline group, an isoquinoline group, a quinoxaline group, a quinazoline group, a phenanthroline group, a pyrazole group, an imidazole group, a triazole group, an oxazole group, an isoxazole group, a thiazole group, an isothiazole group, an oxadiazole group, a thiadiazole group, a benzopyrazole group, a benzimidazole group, a benzoxazole group, a benzothiazole group, a benzoxadiazole group, or a benzothiadiazole group.

The term “a π electron-deficient nitrogen-containing C₁-C₆₀ cyclic group, a π electron-rich C₃-C₆₀ cyclic group, a C₅-C₆₀ cyclic group, and a C₁-C₆₀ heterocyclic group” may be part of a condensed cycle or may be a monovalent, a divalent, a trivalent, a tetravalent, a pentavalent, or a hexavalent group, depending on the formula structure.

As used herein, the number of carbons in each group that is substituted (e.g., C₁-C₆₀) excludes the number of carbons in the substituent. For example, a C₁-C₆₀ alkyl group can be substituted with a C₁-C₆₀ alkyl group. The total number of carbons included in the C₁-C₆₀ alkyl group substituted with the C₁-C₆₀ alkyl group is not limited to 60 carbons. In addition, more than one C₁-C₆₀ alkyl substituent may be present on the C₁-C₆₀ alkyl group. This definition is not limited to the C₁-C₆₀ alkyl group and applies to all substituted groups that recite a carbon range.

In the present specification, at least one substituent of the substituted C₅-C₃₀ carbocyclic group, the substituted C₁-C₃₀ heterocyclic group, the substituted C₁-C₆₀ alkyl group, the substituted C₂-C₆₀ alkenyl group, the substituted C₂-C₆₀ alkynyl group, the substituted C₁-C₆₀ alkoxy group, the substituted C₃-C₁₀ cycloalkyl group, the substituted C₁-C₁₀ heterocycloalkyl group, the substituted C₃-C₁₀ cycloalkenyl group, the substituted C₂-C₁₀ heterocycloalkenyl group, the substituted C₆-C₆₀ aryl group, the substituted C₆-C₆₀ aryloxy group, the substituted C₆-C₆₀ arylthio group, the substituted C₁-C₆₀ heteroaryl group, the substituted monovalent aromatic condensed polycyclic group, the substituted monovalent aromatic condensed heteropolycyclic group, the substituted monovalent non-aromatic condensed polycyclic group, and the substituted monovalent non-aromatic condensed heteropolycyclic group may be:
deuterium, —F, —Cl, —Br, —I, —CD₃, —CD₂H, —CDH₂, —CF₃, —CF₂H, —CFH₂, a hydroxyl group, a cyano group, a nitro group, an amino group, an amidino group, a hydrazine group, a hydrazone group, a carboxylic acid or a salt thereof, a sulfonic acid or a salt thereof, a phosphoric acid or a salt thereof, a C₁-C₆₀ alkyl group, a C₂-C₆₀ alkenyl group, a C₂-C₆₀ alkynyl group, and a C₁-C₆₀ alkoxy group;
a C₁-C₆₀ alkyl group, a C₂-C₆₀ alkenyl group, a C₂-C₆₀ alkynyl group, and a C₁-C₆₀ alkoxy group, each substituted with at least one deuterium, —F, —Cl, —Br, —I, —CD₃, —CD₂H, —CDH₂, —CF₃, —CF₂H, —CFH₂, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a C₃-C₁₀ cycloalkyl group, a C₁-C₁₀ heterocycloalkyl group, a C₃-C₁₀ cycloalkenyl group, a C₂-C₁₀ heterocycloalkenyl group, a C₆-C₆₀ aryl group, a C₆-C₆₀ aryloxy group, a C₆-C₆₀ arylthio group, a C₁-C₆₀ heteroaryl group, a monovalent aromatic condensed polycyclic group, a monovalent aromatic condensed heteropolycyclic group, a monovalent non-aromatic condensed polycyclic group, a monovalent non-aromatic condensed heteropolycyclic group, —N(Q₁₁)(Q₁₂), —Si(Q₁₃)(Q₁₄)(Q₁₅), —B(Q₁₆)(Q₁₇), —P(═O)(Q₁₈)(Q₁₉), or a combination thereof;
a C₃-C₁₀ cycloalkyl group, a C₁-C₁₀ heterocycloalkyl group, a C₃-C₁₀ cycloalkenyl group, a C₂-C₁₀ heterocycloalkenyl group, a C₆-C₆₀ aryl group, a C₆-C₆₀ aryloxy group, a C₆-C₆₀ arylthio group, a C₁-C₆₀ heteroaryl group, a monovalent aromatic condensed polycyclic group, a monovalent aromatic condensed heteropolycyclic group, a monovalent non-aromatic condensed polycyclic group, or a monovalent non-aromatic condensed heteropolycyclic group;
a C₃-C₁₀ cycloalkyl group, a C₁-C₁₀ heterocycloalkyl group, a C₃-C₁₀ cycloalkenyl group, a C₂-C₁₀ heterocycloalkenyl group, a C₆-C₆₀ aryl group, a C₆-C₆₀ aryloxy group, a C₆-C₆₀ arylthio group, a C₁-C₆₀ heteroaryl group, a monovalent aromatic condensed polycyclic group, a monovalent aromatic condensed heteropolycyclic group, a monovalent non-aromatic condensed polycyclic group, or a monovalent non-aromatic condensed heteropolycyclic group, each substituted with at least one of deuterium, —F, —Cl, —Br, —I, —CD₃, —CD₂H, —CDH₂, —CF₃, —CF₂H, —CFH₂, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a C₁-C₆₀ alkyl group, a C₂-C₆₀ alkenyl group, a C₂-C₆₀ alkynyl group, a C₁-C₆₀ alkoxy group, a C₃-C₁₀ cycloalkyl group, a C₁-C₁₀ heterocycloalkyl group, a C₃-C₁₀ cycloalkenyl group, a C₂-C₁₀ heterocycloalkenyl group, a C₆-C₆₀ aryl group, a C₆-C₆₀ aryloxy group, a C₆-C₆₀ arylthio group, a C₁-C₆₀ heteroaryl group, a monovalent aromatic condensed polycyclic group, a monovalent aromatic condensed heteropolycyclic group, a monovalent non-aromatic condensed polycyclic group, a monovalent non-aromatic condensed heteropolycyclic group, —N(Q₂₁)(Q₂₂), —Si(Q₂₃)(Q₂₄)(Q₂₅), —B(Q₂₆)(Q₂₇), —P(═O)(Q₂₈)(Q₂₉), or a combination thereof; or
—N(Q₃₁)(Q₃₂), —Si(Q₃₃)(Q₃₄)(Q₃₅), —B(Q₃₆)(Q₃₇), or —P(═O)(Q₃₈)(Q₃₉),
wherein Q₁ to Q₉, Q₁₁ to Q₁₉, Q₂₁ to Q₂₉, and Q₃₁ to Q₃₉ may each independently be hydrogen, deuterium, —F, —Cl, —Br, —I, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a C₁-C₆₀ alkyl group, a C₂-C₆₀ alkenyl group, a C₂-C₆₀ alkynyl group, a C₁-C₆₀ alkoxy group, a C₃-C₁₀ cycloalkyl group, a C₁-C₁₀ heterocycloalkyl group, a C₃-C₁₀ cycloalkenyl group, a C₂-C₁₀ heterocycloalkenyl group, a C₆-C₆₀ aryl group, a C₆-C₆₀ aryl group substituted with at least one a C₁-C₆₀ alkyl group and a C₆-C₆₀ aryl group, a C₆-C₆₀ aryloxy group, a C₆-C₆₀ arylthio group, a C₁-C₆₀ heteroaryl group, a monovalent aromatic condensed polycyclic group, a monovalent aromatic condensed heteropolycyclic group, a monovalent non-aromatic condensed polycyclic group, or a monovalent non-aromatic condensed heteropolycyclic group.

The term “room temperature” used herein refers to a temperature of about 25° C.

The terms “a biphenyl group, a terphenyl group, and a tetraphenyl group” used herein respectively refer to monovalent groups in which two, three, or four phenyl groups which are linked together via a single bond.

The terms “a cyano-containing phenyl group, a cyano-containing biphenyl group, a cyano-containing terphenyl group, and a cyano-containing tetraphenyl group” used herein respectively refer to a phenyl group, a biphenyl group, a terphenyl group, and a tetraphenyl group, each of which is substituted with at least one cyano group. In “a cyano-containing phenyl group, a cyano-containing biphenyl group, a cyano-containing terphenyl group, and a cyano-containing tetraphenyl group”, a cyano group may be substituted to any position of the corresponding group, and the “cyano-containing phenyl group, the cyano-containing biphenyl group, the cyano-containing terphenyl group, and the cyano-containing tetraphenyl group” may further include substituents other than a cyano group. For example, a phenyl group substituted with a cyano group, and a phenyl group substituted with a cyano group and a methyl group may all belong to “a cyano-containing phenyl group.”

Hereinafter, a compound and an organic light-emitting device according to embodiments are described in detail with reference to Synthesis Examples and Examples. However, the organic light-emitting device is not limited thereto. The wording ‘“B’ was used instead of ‘A″’ used in describing Synthesis Examples means that an amount of ‘A’ used was identical to an amount of ‘B’ used, in terms of a molar equivalent.

### Examples

### Synthesis Example 1: Synthesis of Compound 1

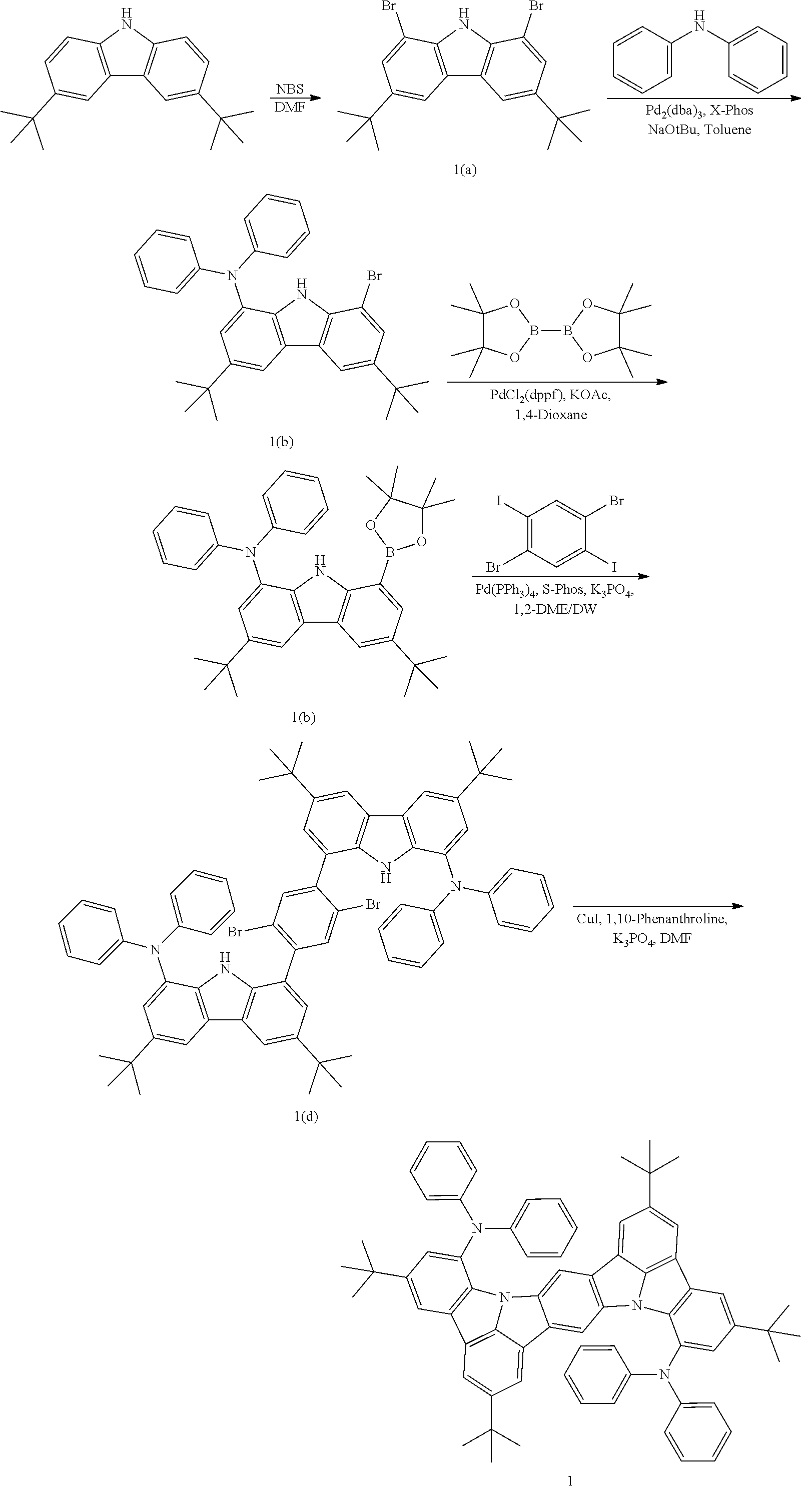

### Synthesis of Intermediate 1(a)

3,6-di-tert-butyl-9H-carbazole (20.0 g, 71.6 mmol) was dissolved in 40 ml of N,N-dimethylformamide (DMF) in a round-bottom flask, and the mixed solution was cooled and stirred at 0° C. Next, N-bromosuccinimide (26.1 g, 147 mmol) dissolved in 60 ml of DMF was slowly added dropwise thereto, and the reaction was carried out while stirring at room temperature. After completion of the reaction, 2 moles of an aqueous sodium thiosulfate solution were added to the reaction mixture, and an extraction process was performed thereon by using distilled water and dichloromethane (DCM). The aqueous solution layer was removed therefrom, and a resulting filtrate was concentrated under pressure. The product thus obtained was separated by column chromatography to obtain 28.2 g (yield of 90%) of Intermediate 1(a) which was a white solid.

LC-Mass (calculated: 280.21 g/mol, found: 281.21 (M+1)).

### Synthesis of Intermediate 1(b)

Intermediate 1(a) (9.69 g, 22.2 mmol), diphenylamine (1.50 g, 8.86 mmol), tris(dibenzylideneacetone)dipalladium(0) (Pd₂(dba)₃) (0.243 g, 0.266 mmol), X-Phos (0.254 g, 0.532 mmol), sodium tert-butoxide (NaOtBu) (1.70 g, 17.7 mmol), and 80 ml of toluene were added to a round flask, and the mixed solution was stirred at 150° C. After completion of the reaction, DCM was added to the reactor cooled to room temperature to dilute the solution, and then passed through a filter filled with silica gel for filtration under reduced pressure. A filtrate thus obtained was concentrated under reduced pressure, adsorbed on silica gel, and separated by silica gel column chromatography, to obtain 2.42 g (yield of 52%) Intermediate 1(b) which is a white solid.

LC-Mass (calculated: 525.19 g/mol, found: 526.09 (M+1)).

### Synthesis of Intermediate 1(c)

Intermediate 1(b) (3.50 g, 6.66 mmol), bis(pinacolato)diboron (B₂pin2) (2.54 g, 9.99 mmol), [1,1′-bis(diphenylphosphino)ferrocene]dichloropalladium(II) (PdCl₂(dppf)) (0.487 g, 0.666 mmol), potassium acetate (KOAc) (3.68 g, 37.5 mmol), and 70 ml of 1,4-dioxane were added to a round flask and stirred under reflux at 120° C. in a nitrogen atmosphere. After completion of reaction, an organic solvent layer was concentrated and purified by column chromatography to obtain 1.11 g (yield of 68%) of Intermediate 1(c).

LC-Mass (calculated: 572.36 g/mol, found: 573.25 (M+1)).

### Synthesis of Intermediate 1(d)

Intermediate 1(c) (2.50 g, 4.36 mmol) 1,4-dibromo-2,5-diiodobenzene (0.850 g, 1.74 mmol), tetrakis(triphenylphosphine)palladium(0) (Pd(PPh₃)₄) (0.201 g, 0.174 mmol), S-Phos (0.143 g, 0.349 mmol), and 20 ml of 1,2-dimethoxyethane (1,2-DME) were added to a round flask and stirred. 10 ml of 4 moles of an aqueous potassium phosphate tribasic (K₃PO₄) solution (8.49 g, 40 mmol) was added to the reaction mixture, and the resulting mixed solution was heated and stirred at 80° C. After completion of the reaction, an extraction process was performed by using distilled water and dichloromethane on the mixed solution cooled to room temperature, and residual water was removed with anhydrous magnesium sulfate, and the resulting product was filtered under reduced pressure. An organic layer thus obtained was concentrated under reduced pressure, and a solid thus obtained was separated and purified through column chromatography to obtain 0.976 g (yield of 50%) of Intermediate 1(d).

LC-Mass (calculated: 1123.39 g/mol, found: 1124.00 (M+1)).

### Synthesis of Compound 1

Intermediate 1(d) (0.13 g, 0.12 mmol), copper(I) iodide (CuI) (0.022 g, 0.12 mmol), 1,10-phenanthroline (0.021 g, 0.12 mmol), phosphate potassium (0.098 g, 0.46 mmol), and 5 ml of DMF were heated and stirred at 100° C. After completion of the reaction, DCM was added to the reactor cooled to room temperature to dilute the solution, and then passed through a filter filled with silica gel for filtration under reduced pressure. A filtrate thus obtained was concentrated under reduced pressure, adsorbed on silica gel, and separated by silica gel column chromatography. DCM and methanol (MeOH) were used to filter the precipitate, and a solid thus obtained was dried in a vacuum oven to obtain 0.076 g (yield of 69%) of Compound 1 which was a yellow solid.

LC-Mass (calculated: 963.5366 g/mol, found: 964.5367 (M+1)).

### Synthesis Example 2: Synthesis of Compound 2

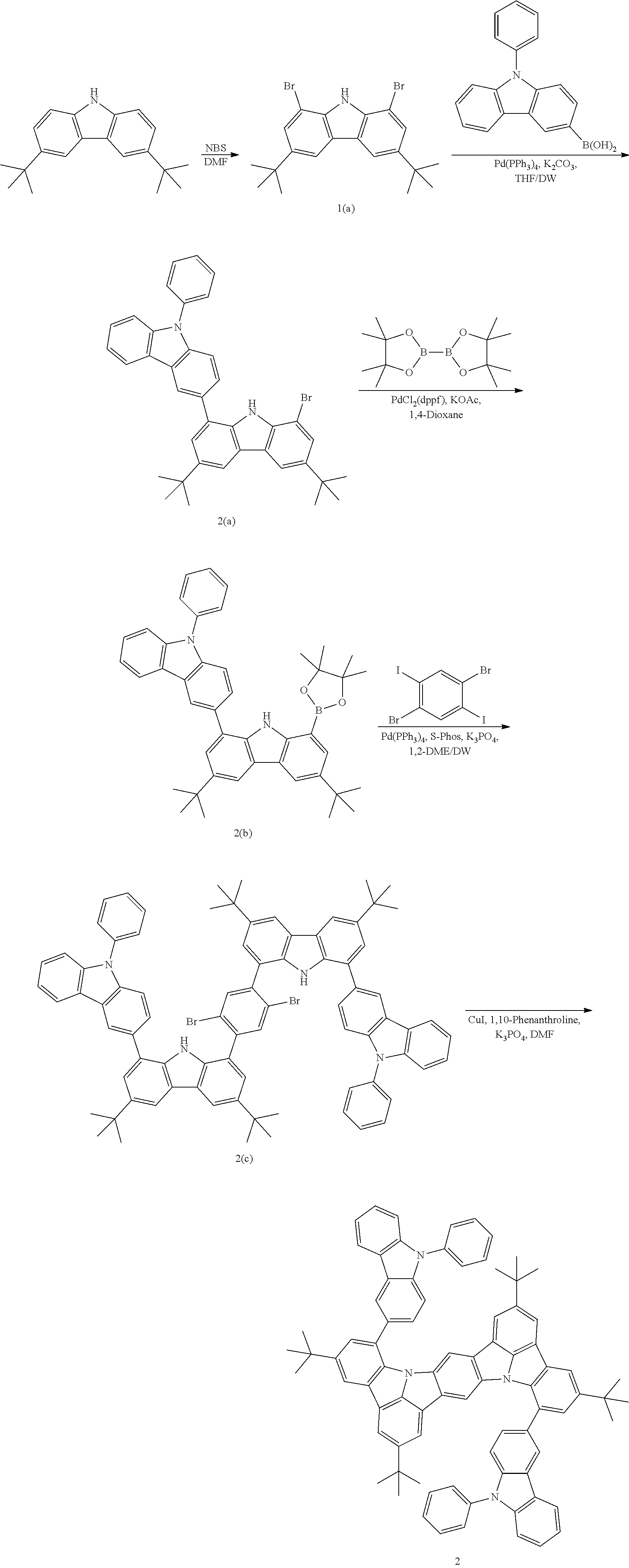

### Synthesis of Intermediate 2(a)

Intermediate 1(a) (11.7 g, 26.8 mmol), (9-phenyl-9H-carbazol-3-yl)boronic acid) (5.12 g, 17.8 mmol), Pd(PPh₃)₄ (0.618 g, 0.178 mmol), and 100 ml of tetrahydrofuran (THF) were added to a round flask and stirred. 50 ml of 2 moles of an aqueous potassium carbonate (K₂CO₃) (13.8 g, 100 mmol) solution was added thereto and stirred under reflux at 140° C. After completion of the reaction, DCM was added to the reactor cooled to room temperature to dilute the solution, and then passed through a filter filled with silica gel for filtration under reduced pressure. A filtrate thus obtained was concentrated under reduced pressure, adsorbed on silica gel, and separated by silica gel column chromatography. DCM and methanol (MeOH) were used to filter the precipitate, and a solid thus obtained was dried in a vacuum oven to obtain 6.12 g (yield of 57%) of Intermediate 2(a) which was a white solid.

LC-Mass (calculated: 599.21 g/mol, found: 600.11 (M+1)).

### Synthesis of Intermediate 2(b)

Intermediate 2(b) (14.6 g, yield of 87%) was obtained in the same manner as used to prepare Intermediate 1(c) in Synthesis Example 1, except that Intermediate 2(a) (15.5 g, 25.9 mmol) was used instead of Intermediate 1(b).

LC-Mass (calculated: 647.38 g/mol, found: 648.25 (M+1)).

### Synthesis of Intermediate 2(c)

Intermediate 2(c) (0.981 g, yield of 38%) was obtained in the same manner as used to prepare Intermediate 1(d) in Synthesis Example 1, except that Intermediate 2(b) (3.32 g, 5.13 mmol) was used instead of Intermediate 1(c).

LC-Mass (calculated: 1271.42 g/mol, found: 1272.12 (M+1)).

### Synthesis of Compound 2

Compound 2 (0.766 g, yield of 88%) was obtained in the same manner as used to prepare Compound 1 in Synthesis Example 11, except that Intermediate 2(c) (1.00 g, 0.785 mmol) was used instead of Intermediate 1(d).

LC-Mass (calculated: 1111.5679 g/mol, found: 1112.5677 (M+1)).

### Synthesis Example 3: Synthesis of Compound 3

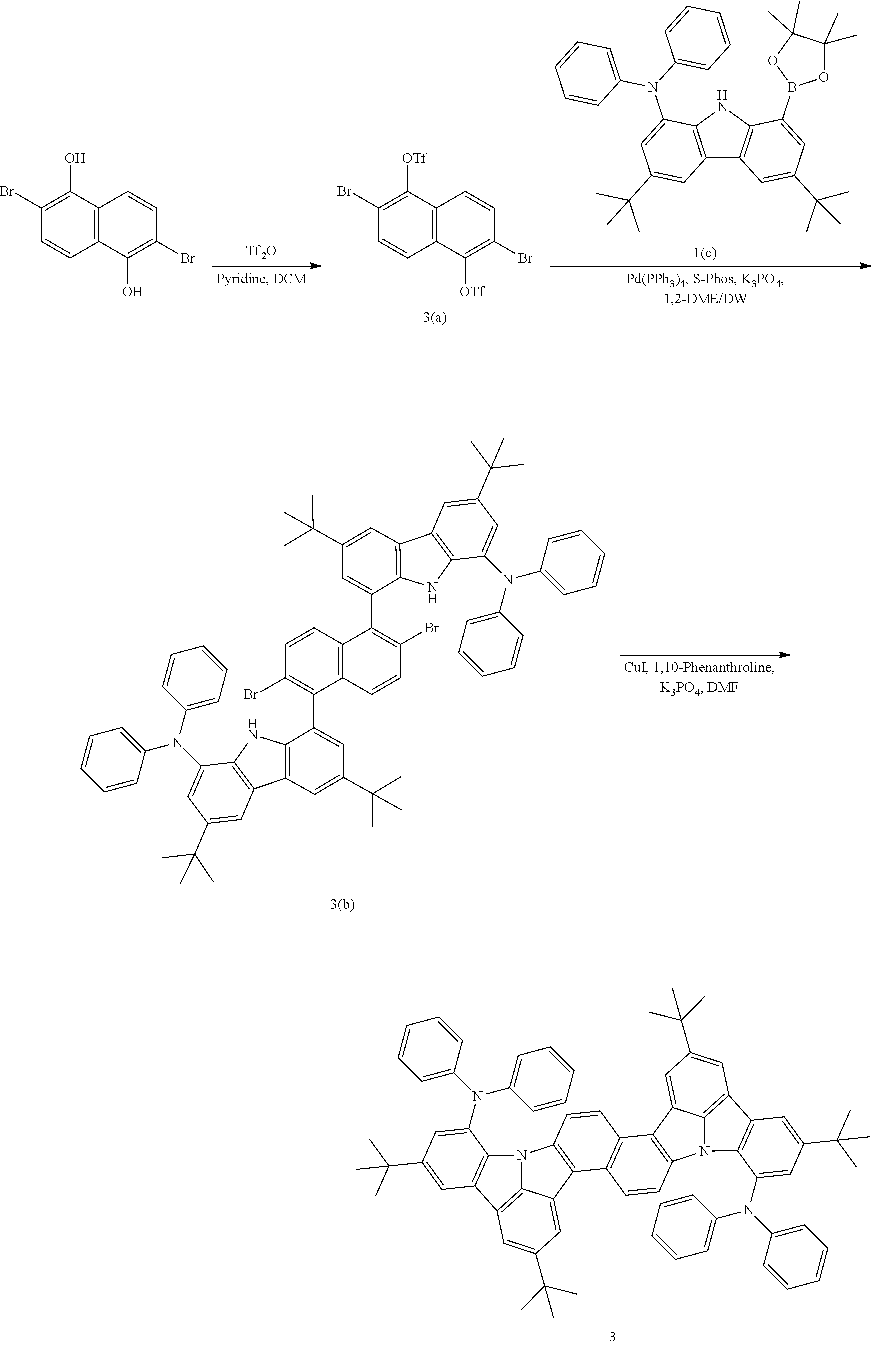 [SMILES: "Tf2O" ; Oc1c(Br)ccc2c(O)c(Br)ccc12 ; O=S(=O)(Oc1c(Br)ccc2c(OS(=O)(=O)C(F)(F)F)c(Br)ccc12)C(F)(F)F ; CC(C)(C)c1cc(B2OC(C)(C)C(C)(C)O2)c2[nH]c3c(N(c4ccccc4)c4ccccc4)cc(C(C)(C)C)cc3c2c1 ; CC(C)(C)c1cc(-c2c(Br)ccc3c(-c4cc(C(C)(C)C)cc5c4[nH]c4c(N(c6ccccc6)c6ccccc6)cc(C(C)(C)C)cc45)c(Br)ccc23)c2[nH]c3c(N(c4ccccc4)c4ccccc4)cc(C(C)(C)C)cc3c2c1 ; CC(C)(C)c1cc(N(c2ccccc2)c2ccccc2)c2c(c1)c1cc(C(C)(C)C)cc3c4c5ccc6c(c5ccc4n2c13)c1cc(C(C)(C)C)cc2c3cc(C(C)(C)C)cc(N(c4ccccc4)c4ccccc4)c3n6c21]

### Synthesis of Intermediate 3(a)

2,6-dibromonaphthalene-1,5-diol (5.00 g, 15.7 mmol) and pyridine (7.60 ml, 94.4 mmol) were dissolved in 60 ml of DCM, and the mixed solution was cooled at stirred at 0° C. for 30 minutes. Trifluoromethanesulfonic anhydride (7.94 ml, 47.2 mmol) was slowly added dropwise to the mixed solution, and a reaction was carried out while stirring at room temperature for 8 hours. An extraction process was performed by using distilled water and dichloromethane on the mixed solution cooled to room temperature, and residual water was removed with anhydrous magnesium sulfate, and the resulting product was filtered under reduced pressure. An organic layer thus obtained was concentrated under reduced pressure, and a solid thus obtained was separated and purified through column chromatography to obtain 5.68 g (yield of 62%) of Intermediate 3(a).

LC-Mass (calculated: 580.78 g/mol, found: 581.80 (M+1)).

### Synthesis of Intermediate 3(b)

Intermediate 3(b) (2.93 g, yield 29%) was synthesized in the same manner as used to prepare Intermediate 1(d) in Synthesis Example 1, except that Intermediate 3(a) (5.00 g, 8.59 mmol) was used instead of 1,4-dibromo-2,5-diiodobenzene.

LC-Mass (calculated: 1173.40 g/mol, found: 1174.41 (M+1)).

### Synthesis of Compound 3

Compound 3 (1.52 g, yield of 88%) was obtained in the same manner as used to prepare Compound 1 in Synthesis Example 11, except that Intermediate 3(b) (2.00 g, 1.70 mmol) was used instead of Intermediate 1(d).

LC-Mass (calculated: 1013.5522 g/mol, found: 1014.5522 (M+1)).

### Synthesis Example 4: Synthesis of Compound 4

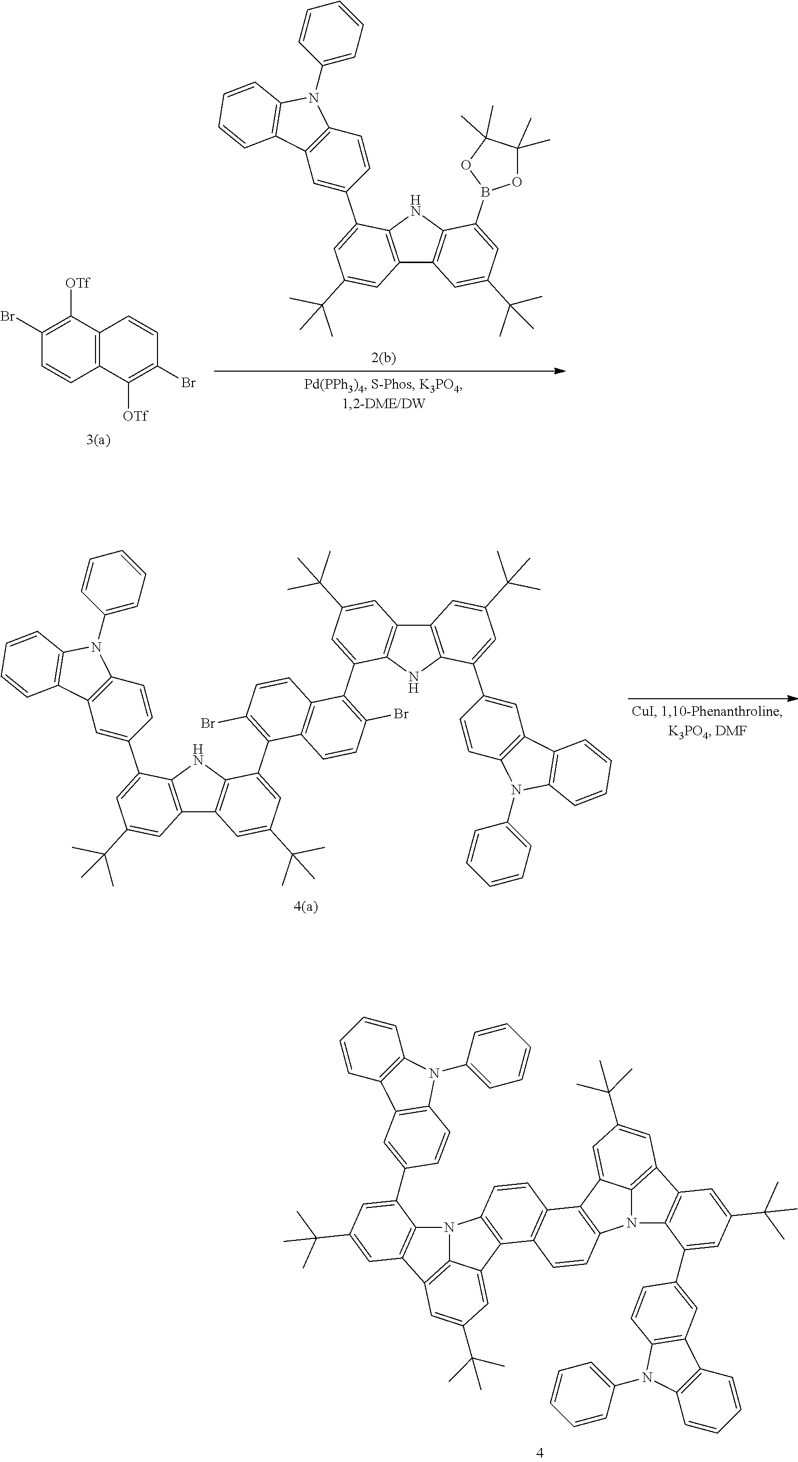

### Intermediate 4(a)

Intermediate 4(a) (4.26 g, yield 25%) was synthesized in the same manner as used to prepare Intermediate 2(c) in Synthesis Example 2, except that Intermediate 3(a) (7.50 g, 12.9 mmol) was used instead of 1,4-dibromo-2,5-diiodobenzene.

LC-Mass (calculated: 1321.44 g/mol, found: 1322.46 (M+1)).

### Synthesis of Compound 4

Compound 4 (2.21 g, yield of 84%) was obtained in the same manner as used to prepare Compound 2 in Synthesis Example 2, except that Intermediate 4(a) (3.00 g, 2.27 mmol) was used instead of Intermediate 2(c).

LC-Mass (calculated: 1161.5835 g/mol, found: 1162.5835 (M+1)).

### Synthesis Example 5: Synthesis of Compound 5

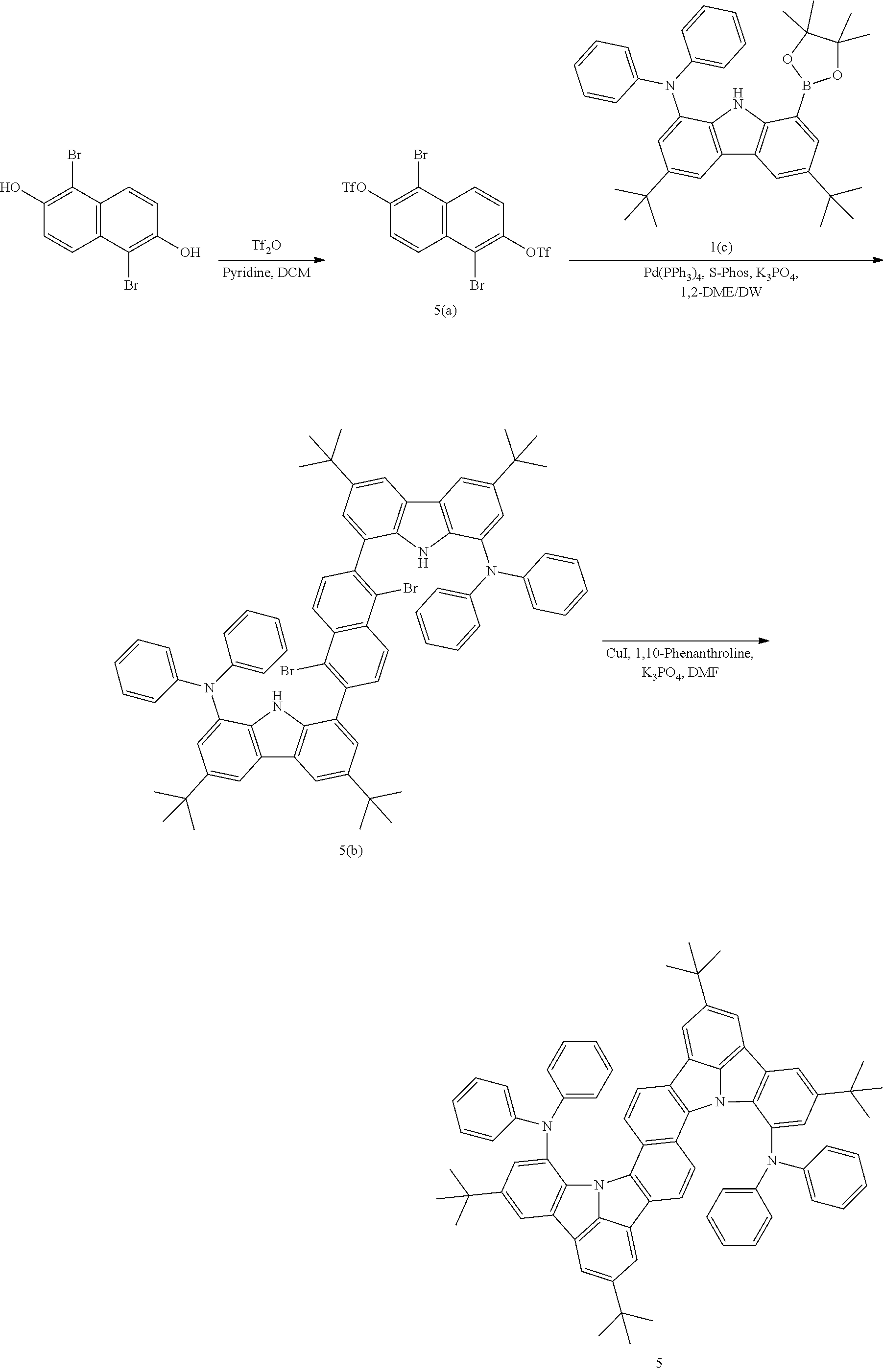 [SMILES: "Tf2O" ; Oc1ccc2c(Br)c(O)ccc2c1Br ; O=S(=O)(Oc1ccc2c(Br)c(OS(=O)(=O)C(F)(F)F)ccc2c1Br)C(F)(F)F ; CC(C)(C)c1cc(B2OC(C)(C)C(C)(C)O2)c2[nH]c3c(N(c4ccccc4)c4ccccc4)cc(C(C)(C)C)cc3c2c1 ; CC(C)(C)c1cc(-c2ccc3c(Br)c(-c4cc(C(C)(C)C)cc5c4[nH]c4c(N(c6ccccc6)c6ccccc6)cc(C(C)(C)C)cc45)ccc3c2Br)c2[nH]c3c(N(c4ccccc4)c4ccccc4)cc(C(C)(C)C)cc3c2c1 ; CC(C)(C)c1cc(N(c2ccccc2)c2ccccc2)c2c(c1)c1cc(C(C)(C)C)cc3c4ccc5c(ccc6c7cc(C(C)(C)C)cc8c9cc(C(C)(C)C)cc(N(c%10ccccc%10)c%10ccccc%10)c9n(c87)c65)c4n2c13]

### Synthesis of Intermediate 5(a)

Intermediate 5(a) (6.13 g, yield 67%) was obtained in the same manner as used to prepare Intermediate 3(a) in Synthesis Example 3, except that 1,5-dibromonaphthalene-2,6-diol (5.00 g, 15.7 mmol) was used instead of 2,6-dibromonaphthalene-1,5-diol.

LC-Mass (calculated: 580.78 g/mol, found: 581.78 (M+1)).

### Synthesis of Intermediate 5(b)

Intermediate 5(b) (3.43 g, yield 34%) was synthesized in the same manner as used to prepare Intermediate 1(d) in Synthesis Example 1, except that Intermediate 5(a) (7.00 g, 8.59 mmol) was used instead of 1,4-dibromo-2,5-diiodobenzene.

LC-Mass (calculated: 1173.40 g/mol, found: 1174.40 (M+1)).

### Synthesis of Compound 5

Compound 5 (2.40 g, yield of 81%) was obtained in the same manner as used to prepare Compound 1 in Synthesis Example 11, except that Intermediate 5(b) (3.00 g, 2.55 mmol) was used instead of Intermediate 1(d).

LC-Mass (calculated: 1013.5522 g/mol, found: 1014.5522 (M+1)).

### Synthesis Example 6: Synthesis of Compound 6

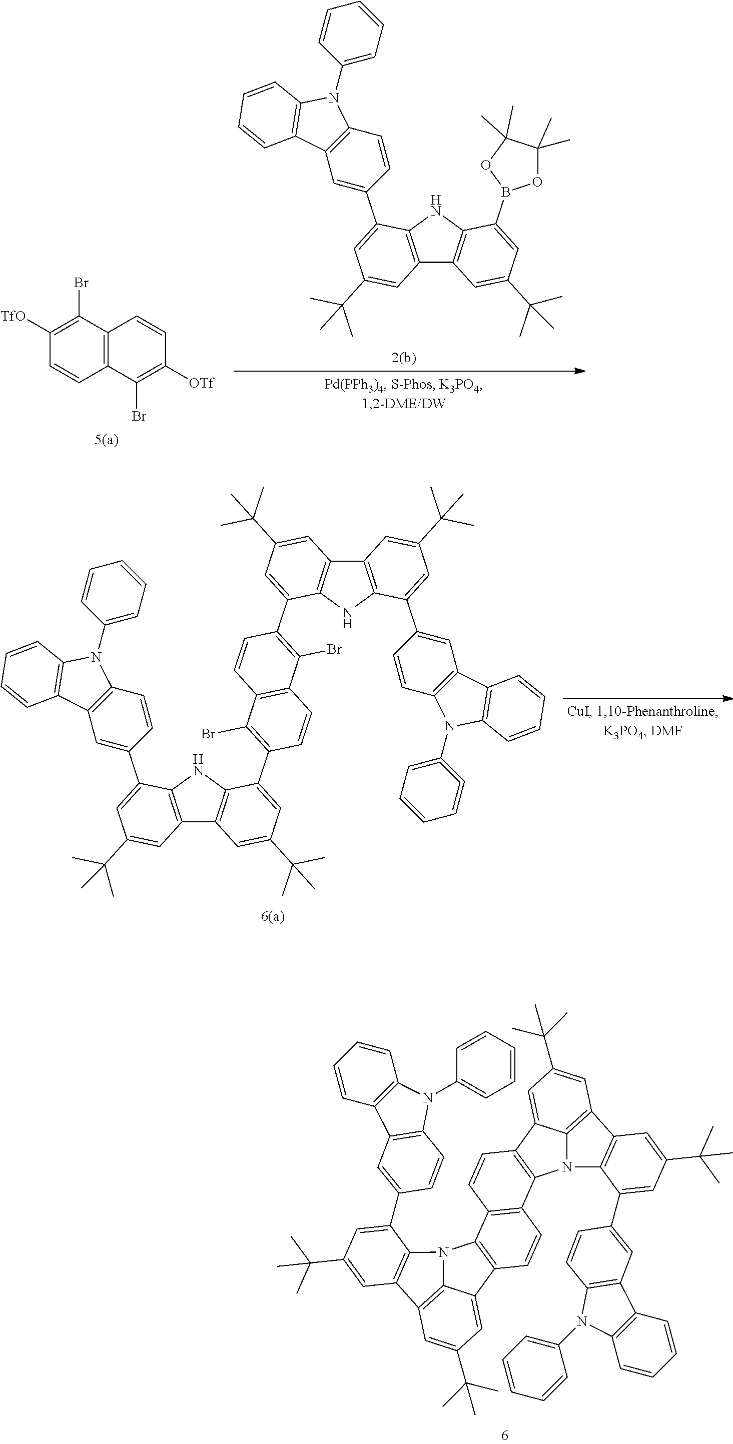

### Synthesis of Intermediate 6(a)

Intermediate 6(a) (4.62 g, yield 29%) was synthesized in the same manner as used to prepare Intermediate 3(a) in Synthesis Example 3, except that Intermediate 5(a) (7.00 g, 12.0 mmol) was used instead of 1,4-dibromo-2,5-diiodobenzene.

LC-Mass (calculated: 1321.44 g/mol, found: 1322.45 (M+1)).

### Synthesis of Compound 6

Compound 6 (3.17 g, yield of 86%) was obtained in the same manner as used to prepare Compound 1 in Synthesis Example 11, except that Intermediate 6(a) (4.20 g, 3.17 mmol) was used instead of Intermediate 1(d).

LC-Mass (calculated: 1161.5835 g/mol, found: 1162.5835 (M+1)).

### Evaluation Example 1: Evaluation of HOMO, LUMO, T₁, and S₁ Energy Levels

For the compounds of Table 8, the HOMO, LUMO, Ti, and Si energy levels were measured according to methods described in Table 7, and results thereof are shown in Table 8:

TABLE 7

| | |
|---|---|
| HOMO | By using cyclic voltammetry (CV) (electrolyte: 0.1M |
| energy | TBAP/solvent: ACN/electrode: 3-electrode system (working |
| level | electrode: Pt wire, reference electrode: Ag/AgCl, and auxiliary |
| evaluation | electrode: Pt wire)), the potential (V)-current (A) graph |
| method | of each compound was obtained, and then, from the oxidation |
| | onset of the graph, the HOMO energy level of each compound |
| | was calculated. |
| LUMO | Each compound was diluted in THF to a concentration of |
| energy | 1 × 10⁻⁵ M, and the UV absorption spectrum thereof was |
| level | measured at room temperature by using a JASCO V-730 UV |
| evaluation | spectrometer. Then, the lowest unoccupied molecular orbital |
| method | (LUMO) energy level was calculated by using the optical |
| | band gap (Eg) from the edge of the absorption spectrum |
| | and HOMO energy level. |
| T₁ energy | Each compound was dissolved in THF to a |
| method | concentration of 1 × 10⁻⁵ M, and then placed in a quartz |
| level | cell to lower the temperature by using liquid nitrogen (77 K). |
| evaluation | Afterwards, a photoluminescence spectrum was measured |
| | with a delay time of 1 millisecond by using a |
| | fluorescence spectrometer (PerkinElmer, LS-55). |
| S₁ energy | S₁ energy level was measured similarly to the T₁ energy |
| level | level evaluation method, except that it was measured without |
| evaluation | delay time. Here, S₁ was determined as a maximum emission |
| method | peak point. |

TABLE 8

| | HOMO | LUMO | S1 | T1 | ΔE_{ST} |
|---|---|---|---|---|---|
| Compound No. | (eV) | (eV) | (eV) | (eV) | (eV) |
| 1 (tBisICz-DPA) | −5.65 | −2.89 | 2.79 | 2.51 | 0.28 |
| 2 (tBisICz-PhCz) | −5.67 | −2.89 | 2.82 | 2.51 | 0.31 |
| A | — | — | 2.84 | 2.55 | 0.29 |
| B | −5.56 | −2.93 | 2.68 | 2.32 | 0.36 |
| C | −5.62 | −2.89 | 2.67 | 2.31 | 0.36 |

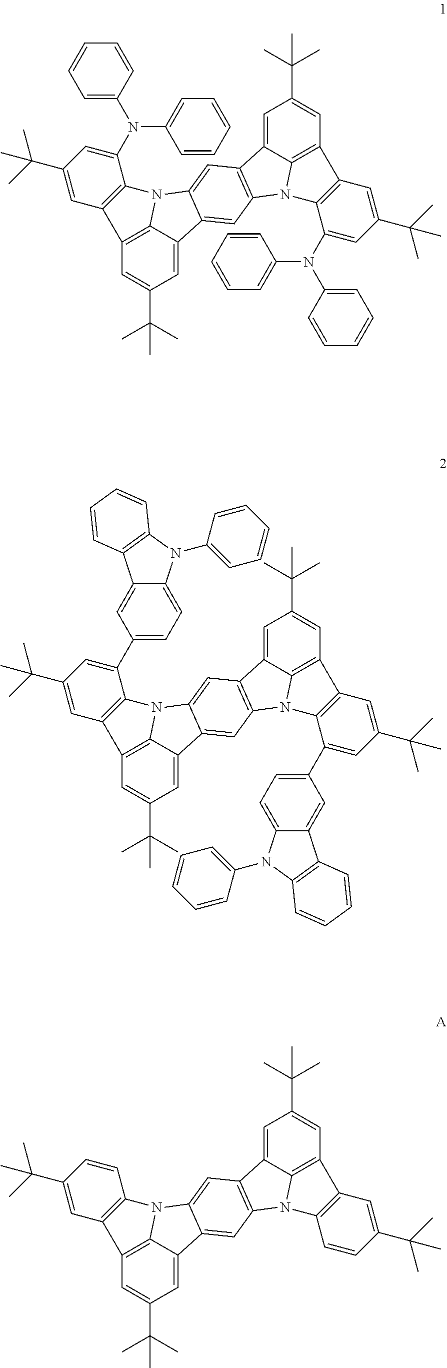

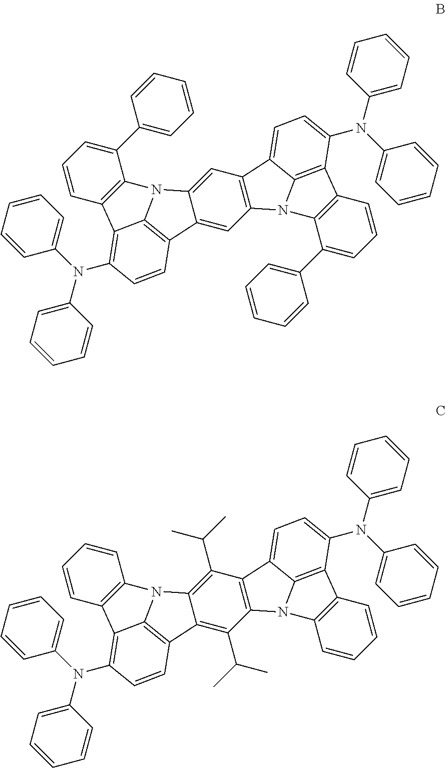 [SMILES: c1ccc(-c2cccc3c4c(N(c5ccccc5)c5ccccc5)ccc5c6cc7c(cc6n(c23)c54)c2ccc(N(c3ccccc3)c3ccccc3)c3c4cccc(-c5ccccc5)c4n7c23)cc1 ; CC(C)c1c2c3ccc(N(c4ccccc4)c4ccccc4)c4c5ccccc5n(c2c(C(C)C)c2c5ccc(N(c6ccccc6)c6ccccc6)c6c7ccccc7n(c12)c56)c34]

Referring to Table 8, it was confirmed that Compounds 1 and 2 each had such electric characteristics that are suitable for use as a dopant for an electronic device, for example, an organic light-emitting device.

### Evaluation Example 2: Measurement of Photoluminescence (PL) of Solution

The resulting Compounds and the Comparative Compound were each dissolved in toluene to prepare a 1×10⁻⁵ M solution. This solution was filled in a 1 cm square four-sided transmission cell, and PL measurement was performed at room temperature using a spectrofluorometer F7000 (available from Hitachi High-Technologies Corporation). A peak wavelength, a FWHM, and a Stokes shift were computed from the obtained emission spectrum. The results of evaluation are shown in Table 10.

TABLE 9

| | Peak wavelength | Stocks shift | FWHM |
|---|---|---|---|
| Compound No. | (nm) | (nm) | (nm) |
| 1 (tBisICz-DPA) | 444 | 17 | 27 |
| 2 (tBisICz-PhCz) | 440 | 12 | 16 |
| A | 436 | 11 | 22 |
| B | 475 | 22 | 29 |
| C | 476 | 24 | 30 |

Referring to Table 8, it was confirmed that Compounds 1 and 2 each had improved color characteristics.

### Example 1-1

An ITO glass substrate was cut to a size of 50 mm×50 mm×0.5 mm and then, sonicated in acetone isopropyl alcohol and pure water, each for 15 minutes, and then, washed by exposure to ultraviolet (UV) light ozone for 30 minutes.

Subsequently, PEDOT:PSS was spin-coated on the ITO electrode (anode) of the glass substrate to form a first hole injection layer having a thickness of 40 nm, TAPC was deposited on the hole injection layer to form a second hole injection layer having a thickness of 5 nm, TCTA was deposited on the second hole injection layer to form a first hole transport layer having a thickness of 5 nm, PCzAc was deposited on the first hole transport layer to form a second hole transport layer having a thickness of 5 nm, and mCP was deposited on the second hole transport layer to form an electron blocking layer having a thickness of 5 nm, thereby forming a hole transport region.

In the hole transport region, mCP (first host), TSPO1 (second host), and Compound 1(dopant) (In this regard, a content of the dopant was about 3 wt % based on the total weight of the first host, the second host, and the dopant) were co-deposited to form an emission layer having a thickness of 25 nm.

TSPO1 was deposited on the emission layer to form an electron transport layer having a thickness of 25 nm, LiF was deposited on the electron transport layer to form an electron injection layer having a thickness of 1.5 nm, and Al was formed on the electron injection layer to a thickness of 200 nm, thereby completing the manufacture of an organic light-emitting device.

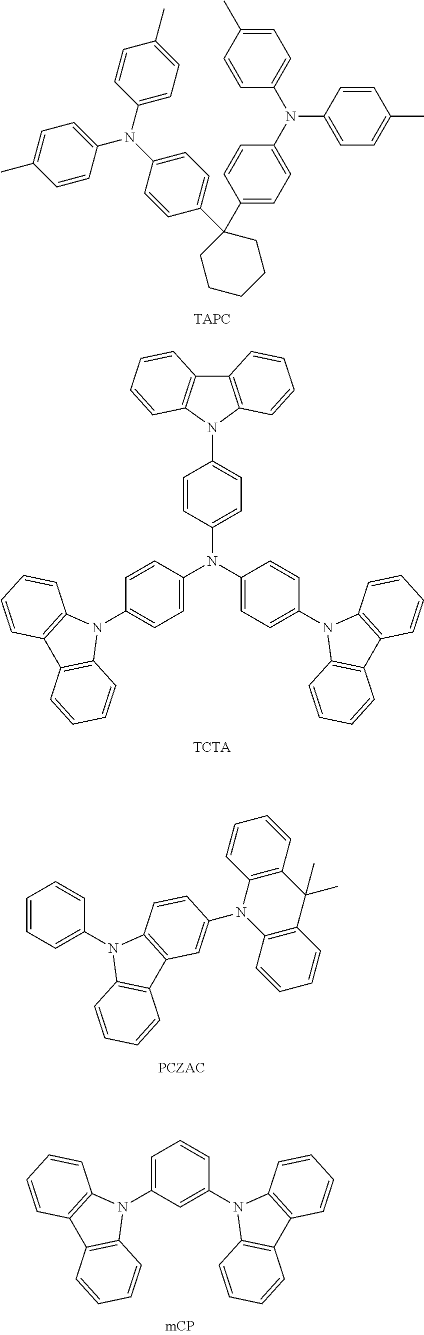

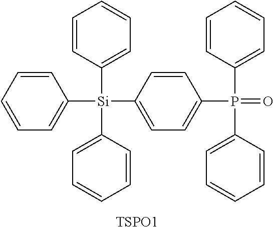

### Examples 1-2 to 1-6 and Comparative Examples 1-1 to 1-3

An organic light-emitting device was manufactured in the same manner as in Example 1-1, except that, for use as a dopant in forming an emission layer, corresponding compounds shown in Table 10 were used.

### Example 2-1

An ITO glass substrate was cut to a size of 50 mm×50 mm×0.5 mm and then, sonicated in acetone isopropyl alcohol and pure water, each for 15 minutes, and then, washed by exposure to ultraviolet (UV) light ozone for 30 minutes.

Subsequently, PEDOT:PSS was spin-coated on the ITO electrode (anode) of the glass substrate to form a hole injection layer having a thickness of 90 nm, TAPC was deposited on the hole injection layer to form a hole transport layer having a thickness of 20 nm, and mCP was deposited on the hole transport layer to form an electron blocking layer having a thickness of 10 nm.

On the hole transport region, mCP (first host), TSPO1 (second host), Pt* (sensitizer), and Compound 1 (dopant) (in this regard, a content of the dopant was about 0.4 wt % based on the total weight of the host, the sensitizer, and the dopant, and a content of the sensitizer was about 13 wt % based on the total weight of the host, the sensitizer, and the dopant) were co-deposited to form an emission layer having a thickness of 25 nm.

TSPO1 was deposited on the emission layer to form a first electron transport layer having a thickness of 5 nm, TPBi was deposited on the first electron transport layer to form a second electron transport layer having a thickness of 20 nm, LiF was deposited on the second electron transport layer to form an electron injection layer having a thickness of 1.5 nm, and Al was formed on the electron injection layer to a thickness of 200 nm, thereby completing the manufacture of an organic light-emitting device.

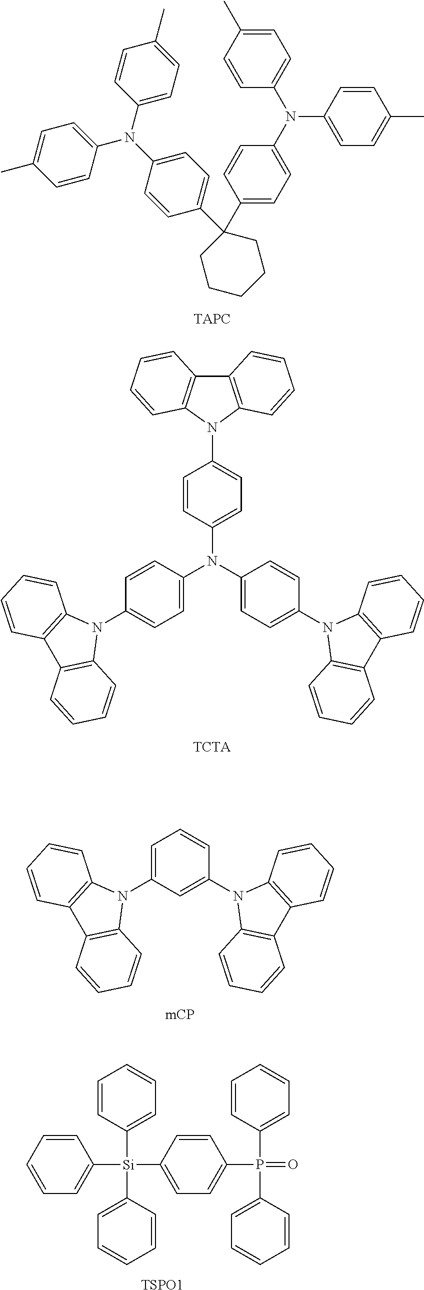

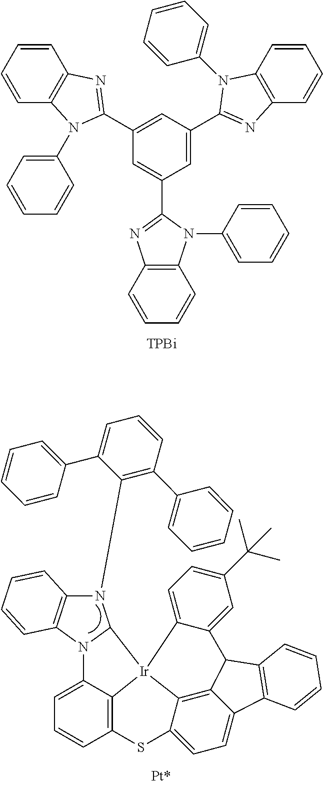

### Examples 2-2 to 2-6 and Comparative Examples 2-1 to 2-3

An organic light-emitting device was manufactured in the same manner as in Example 2-1, except that, for use as a dopant in forming an emission layer, corresponding compounds shown in Table 11 were used.

### Evaluation Example: Evaluation of OLED Characteristics

For the organic light-emitting devices prepared according to Examples and Comparative Examples above, the driving voltage, maximum emission wavelength, CIE y color coordinates (at 1000 nit), FWHM, and relative efficiency were measured by using a current-voltage meter (Keithley 2400) and a luminance meter (Minolta Cs-1000A), and results thereof are summarized in Tables 10 and 11.

TABLE 10

| | Weight | Weight | | | | | | | |
|---|---|---|---|---|---|---|---|---|---|
| | ratio of | ratio of | | Doping | Driving | | | | Relative |
| | first | second | | concentration | voltage | λ_{EL} | FWHM | | efficiency |
| | host | host | Dopant | (wt %) | (V) | (nm) | (nm) | CIE y | (%) |
| Example 1-1 | 50 | 50 | 1 | 3 | 6.5 | 452 | 28 | 0.05 | 110 |
| Example 1-2 | 50 | 50 | 2 | 3 | 6.2 | 446 | 19 | 0.04 | 134 |
| Comparative | 50 | 50 | A | 3 | 5.7 | 448 | 35 | 0.07 | 100 |
| Example 1-1 | | | | | | | | | |
| Comparative | 50 | 50 | B | 3 | 6.5 | 477 | 44 | >0.2 | 105 |
| Example 1-2 | | | | | | | | | |
| Comparative | 50 | 50 | C | 3 | 7.4 | 480 | 46 | >0.2 | 81 |
| Example 1-3 | | | | | | | | | |

TABLE 11

| | Weight | Weight | | | | | | | |
|---|---|---|---|---|---|---|---|---|---|
| | ratio of | ratio of | | Concentration | | Doping | Driving | | Relative |
| | first | second | | of sensitizer | | concentration | voltage | λ_{EL} | efficiency |
| | host | host | Sensitizer | (wt %) | Dopant | (wt %) | (V) | (nm) | (%) |
| Example 2-1 | 50 | 50 | Pt* | 13 | 1 | 0.4 | 4.09 | 462 | 142 |
| Example 2-4 | 50 | 50 | Pt* | 13 | 2 | 0.4 | 3.99 | 462 | 137 |
| Comparative | 50 | 50 | Pt* | 13 | A | 0.4 | 4.35 | 466 | 100 |
| Example 2-1 | | | | | | | | | |
| Comparative | 50 | 50 | Pt* | 13 | B | 0.4 | 4.07 | 479 | 106 |
| Example 2-2 | | | | | | | | | |

Referring to Tables 10 and 11, it was confirmed that the organic light-emitting device of Examples had high efficiency compared to the organic light-emitting devices of Comparative Examples.

According to the one or more embodiments, an organic light-emitting device including a heterocyclic compound may have improved efficiency and/or colorimetric purity.

It should be understood that embodiments described herein should be considered in a descriptive sense only and not for purposes of limitation. Descriptions of features or aspects within each embodiment should typically be considered as available for other similar features or aspects in other embodiments. While one or more embodiments have been described with reference to the figures, it will be understood by those of ordinary skill in the art that various changes in form and details may be made therein without departing from the spirit and scope as defined by the following claims.

What is claimed is:

1. A heterocyclic compound represented by Formula 1: 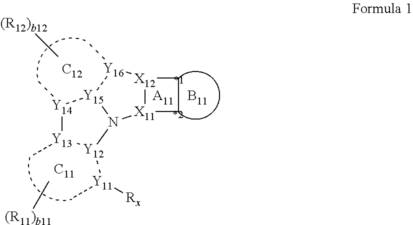
wherein, in Formula 1,
X₁₁ and X₁₂ are each independently a carbon atom, and are linked to each other via a single bond or a double bond,
Y₁₁ to Y₁₆ are each independently a carbon atom, and any neighboring two of Y₁₁ to Y₁₆ are linked via a single bond or a double bond, and
B₁₁ is a group represented by Formula 1A: 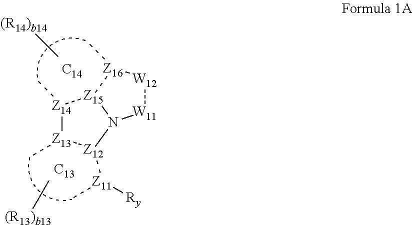
wherein, in Formula 1A,
Z₁₁ to Z₁₆ are each independently a carbon atom, and any neighboring two of Z₁₁ to Z₁₆ are linked via a single bond or a double bond, and
W₁₁ and W₁₂ are each independently a carbon atom, are linked via a single bond or a double bond, and are *1 and *2 in Formula 1,
in Formulae 1 and 1A, A₁₁ and C₁₁ to C₁₄ are each independently a C₅-C₃₀ carbocyclic group or a C₁-C₃₀ heterocyclic group,
Rₓ is a group represented by one of Formulae 2-1 to 2-3,
R_{y} and R₁₁ to R₁₅ are each independently a group represented by one of Formulae 2-1 to 2-3, hydrogen, deuterium, —F, —Cl, —Br, —I, —SF₅, a hydroxyl group, a cyano group, a nitro group, an amidino group, a hydrazino group, a hydrazono group, a substituted or unsubstituted C₁-C₆₀ alkyl group, a substituted or unsubstituted C₂-C₆₀ alkenyl group, a substituted or unsubstituted C₂-C₆₀ alkynyl group, a substituted or unsubstituted C₁-C₆₀ alkoxy group, a substituted or unsubstituted C₃-C₁₀ cycloalkyl group, a substituted or unsubstituted C₁-C₁₀ heterocycloalkyl group, a substituted or unsubstituted C₃-C₁₀ cycloalkenyl group, a substituted or unsubstituted C₂-C₁₀ heterocycloalkenyl group, a substituted or unsubstituted C₆-C₆₀ aryl group, a substituted or unsubstituted C₇-C₆₀ alkylaryl group, a substituted or unsubstituted C₆-C₆₀ aryloxy group, a substituted or unsubstituted C₆-C₆₀ arylthio group, a substituted or unsubstituted C₁-C₆₀ heteroaryl group, a substituted or unsubstituted C₂-C₆₀ heteroalkylaryl group, a substituted or unsubstituted C₁-C₆₀ heteroaryloxy group, a substituted or unsubstituted C₁-C₆₀ heteroarylthio group, a substituted or unsubstituted monovalent non-aromatic condensed polycyclic group, a substituted or unsubstituted monovalent non-aromatic condensed heteropolycyclic group, —Si(Q₁)(Q₂)(Q₃), —C(Q₁)(Q₂)(Q₃), —B(Q₁)(Q₂), —N(Q₁)(Q₂), —P(Q₁)(Q₂), —C(═O)(Q₁), —S(═O)(Q₁), —S(═O)₂(Q₁), —P(═O)(Q₁)(Q₂), or —P(═S)(Q₁)(Q₂),
b11 to b14 are each independently 0, 1, 2, 3, or 4, and
b15 is 0, 1, or 2: 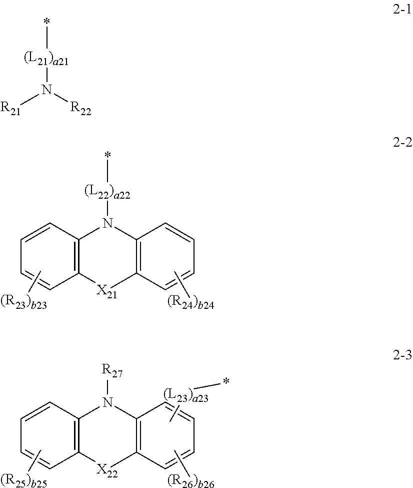
wherein, in Formulae 2-1 to 2-3,
X₂₁ is a single bond, O, S, N(R₂₈), or C(R₂₈)(R₂₉),
X₂₂ is a single bond, O, S, N(R₃₀), or C(R₃₀)(R₃₁),
L₂₁ to L₂₃ are each independently a substituted or unsubstituted C₅-C₃₀ carbocyclic group or a substituted or unsubstituted C₁-C₃₀ heterocyclic group,
a21 to a23 are each independently 0, 1, 2, 3, or 4,
R₂₁ and R₂₂ are each independently a substituted or unsubstituted C₁-C₆₀ alkyl group, a substituted or unsubstituted C₃-C₁₀ cycloalkyl group, a substituted or unsubstituted C₁-C₁₀ heterocycloalkyl group, a substituted or unsubstituted C₃-C₁₀ cycloalkenyl group, a substituted or unsubstituted C₂-C₁₀ heterocycloalkenyl group, a substituted or unsubstituted C₆-C₆₀ aryl group, a substituted or unsubstituted C₇-C₆₀ alkylaryl group, a substituted or unsubstituted C₁-C₆₀ heteroaryl group, a substituted or unsubstituted C₂-C₆₀ heteroalkylaryl group, a substituted or unsubstituted monovalent non-aromatic condensed polycyclic group, or a substituted or unsubstituted monovalent non-aromatic condensed heteropolycyclic group,
R₂₃ to R₃₁ are each independently hydrogen, deuterium, —F, —Cl, —Br, —I, —SF₅, a hydroxyl group, a cyano group, a nitro group, an amidino group, a hydrazine group, a hydrazone group, a substituted or unsubstituted C₁-C₆₀ alkyl group, a substituted or unsubstituted C₂-C₆₀ alkenyl group, a substituted or unsubstituted C₂-C₆₀ alkynyl group, a substituted or unsubstituted C₁-C₆₀ alkoxy group, a substituted or unsubstituted C₃-C₁₀ cycloalkyl group, a substituted or unsubstituted C₁-C₁₀ heterocycloalkyl group, a substituted or unsubstituted C₃-C₁₀ cycloalkenyl group, a substituted or unsubstituted C₂-C₁₀ heterocycloalkenyl group, a substituted or unsubstituted C₆-C₆₀ aryl group, a substituted or unsubstituted C₇-C₆₀ alkylaryl group, a substituted or unsubstituted C₆-C₆₀ aryloxy group, a substituted or unsubstituted C₆-C₆₀ arylthio group, a substituted or unsubstituted C₁-C₆₀ heteroaryl group, a substituted or unsubstituted C₂-C₆₀ heteroalkylaryl group, a substituted or unsubstituted heteroaryloxy group, a substituted or unsubstituted heteroarylthio group, a substituted or unsubstituted monovalent non-aromatic condensed polycyclic group, a substituted or unsubstituted monovalent non-aromatic condensed heteropolycyclic group, —Si(Q₁)(Q₂)(Q₃), —C(Q₁)(Q₂)(Q₃), —B(Q₁)(Q₂), —N(Q₁)(Q₂), —P(Q₁)(Q₂), —C(═O)(Q₁), —S(═O)(Q₁), —S(═O)₂(Q₁), —P(═O)(Q₁)(Q₂), or —P(═S)(Q₁)(Q₂),
b23 to b26 are each independently 0, 1, 2, 3, or 4,
Q₁ to Q₃ are each independently hydrogen, deuterium, —F, —Cl, —Br, —I, a hydroxyl group, a cyano group, a nitro group, an amidino group, a hydrazine group, a hydrazone group, a C₁-C₆₀ alkyl group, a C₂-C₆₀ alkenyl group, a C₂-C₆₀ alkynyl group, a C₁-C₆₀ alkoxy group, a C₃-C₁₀ cycloalkyl group, a C₁-C₁₀ heterocycloalkyl group, a C₃-C₁₀ cycloalkenyl group, a C₂-C₁₀ heterocycloalkenyl group, a C₆-C₆₀ aryl group, a C₇-C₆₀ alkylaryl group, a C₆-C₆₀ aryloxy group, a C₆-C₆₀ arylthio group, a C₁-C₆₀ heteroaryl group, a C₂-C₆₀ heteroalkylaryl group, a C₁-C₆₀ heteroaryloxy group, a C₁-C₆₀ heteroarylthio group, a monovalent non-aromatic condensed polycyclic group, a monovalent non-aromatic condensed heteropolycyclic group, a C₁-C₆₀ alkyl group that is substituted with at least one deuterium, —F, a cyano group, a C₁-C₆₀ alkyl group, or a C₆-C₆₀ aryl group, and a C₆-C₆₀ aryl group that is substituted with at least one deuterium, —F, a cyano group, a C₁-C₆₀ alkyl group, a C₆-C₆₀ aryl group, or any combination thereof, and
* indicates a binding site to a neighboring atom.

2. The heterocyclic compound of claim 1, wherein A₁₁ and C₁₁ to C₁₄ are each independently a benzene group, a naphthalene group, a phenanthrene group, a furan group, a thiophene group, a pyrrole group, a cyclopentene group, a silole group, a germole group, a benzofuran group, a benzothiophene group, an indole group, an indene group, a benzosilole group, a benzogermole group, a dibenzofuran group, a dibenzothiophene group, a carbazole group, a fluorene group, a dibenzosilole group, a dibenzogermole group, a pyridine group, a pyrimidine group, or a pyridazine group.

3. The heterocyclic compound of claim 1, wherein A₁₁ is a group represented by Formula 3-1: 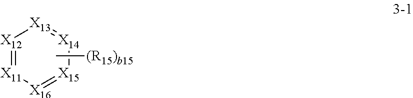
wherein, in Formula 3-1,
X₁₁ to X₁₆ are each independently a nitrogen atom or a carbon atom, and any neighboring two of X₁₃ to X₁₆ are each *1 and *2 in Formula 1,
R₁₅ is defined the same as R₁₁ in Formula 1, and
b15 is 0, 1, or 2.

4. The heterocyclic compound of claim 1, wherein A₁₁ is a group represented by one of Formulae 3-11 to 3-16: 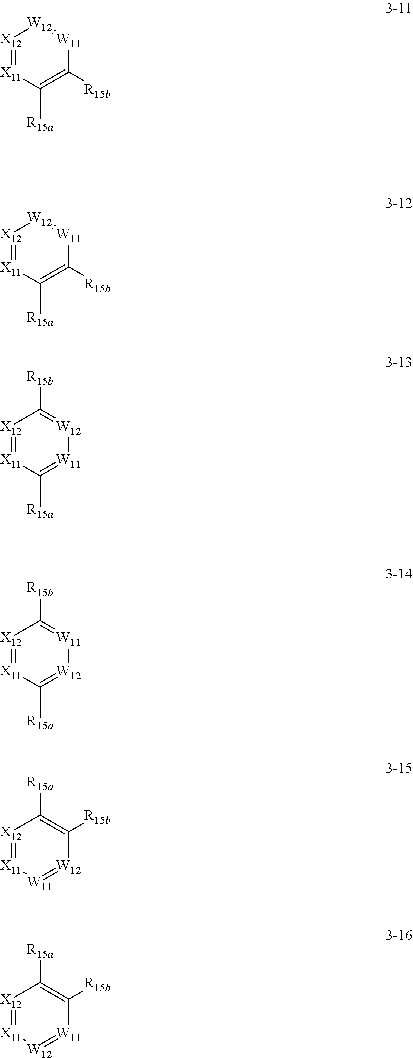
wherein, in Formulae 3-11 to 3-16,
X₁₁ to X₁₆ are each independently a nitrogen atom or a carbon atom,
W₁₁ and W₁₂ are each independently a nitrogen atom or a carbon atom, and
R₁₅ₐ and R_{15b} are each independently defined the same as Ru in Formula 1.

5. The heterocyclic compound of claim 1, wherein Rₓ and R_{y} in Formula 1 are each independently a group represented by one of Formulae 2-1 to 2-3.

6. The heterocyclic compound of claim 1, wherein the heterocyclic compound is represented by Formulae 11 or 12: 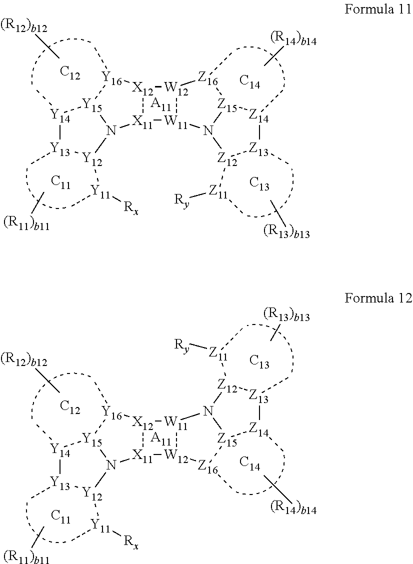
wherein, in Formulae 11 and 12,
X₁₁, X₁₂, Y₁₁ to Y₁₆, Z₁₁ to Z₁₆, W₁₁, W₁₂, A₁₁, C₁₁ to C₁₄, Rₓ, R_{y}, R₁₁ to R₁₄, and b₁₁ to b₁₄ are defined the same as those in Formulae 1 and 1A, respectively.

7. The heterocyclic compound of claim 6, wherein A₁₁ is a group represented by one of Formulae 3-11 to 3-16: 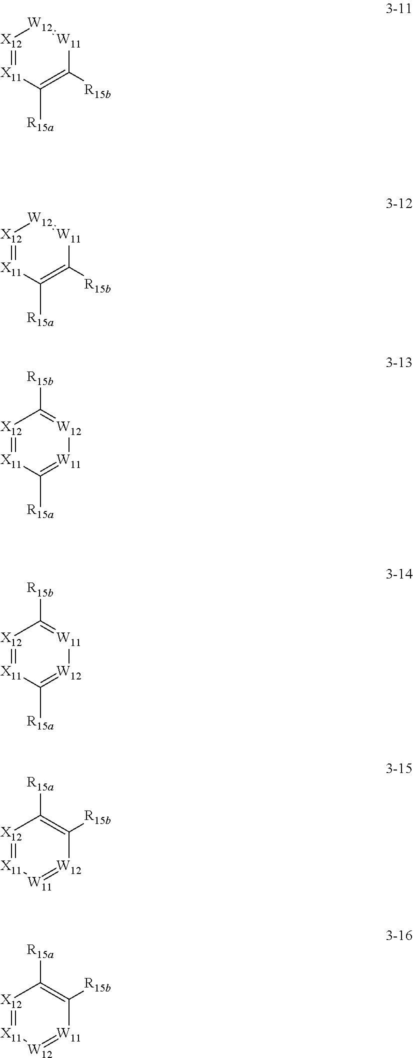
wherein, in Formulae 3-11 to 3-16,
X₁₁ to X₁₆ are each independently a nitrogen atom or a carbon atom,
W₁₁ and W₁₂ are each independently a nitrogen atom or a carbon atom, and
R₁₅ₐ and R_{15b} are each independently defined the same as R₁₁ in Formula 1.

8. The heterocyclic compound of claim 1, wherein the heterocyclic compound is represented by Formulae 11-1 or 12-1: 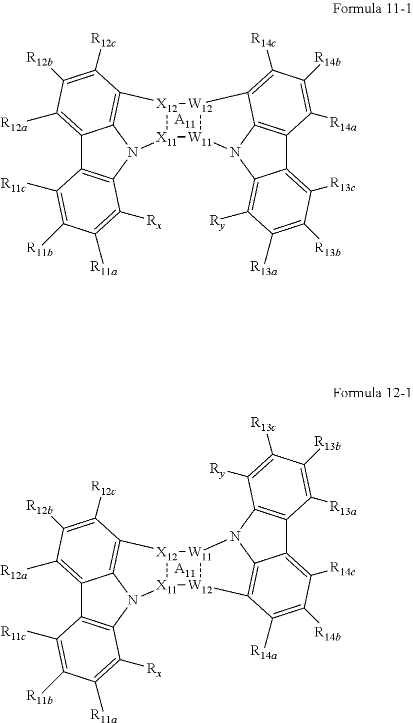
wherein, in Formulae 11-1 and 12-1,
X₁₁, X₁₂, W₁₁, W₁₂, A₁₁, Rₓ, and R_{y} are defined the same as those in Formulae 1 and 1A, respectively,
R₁₁ₐ to R_{11c}, R₁₂ₐ to R_{12c}, R₁₃ₐ to R_{13c}, and R₁₄ₐ to R_{14c} are each defined the same as R₁₁ in Formula 1, and any neighboring two of R₁₁ₐ to R_{11c}, R₁₂ₐ to R_{12c}, R₁₃ₐ to R_{13c}, and R₁₄ₐ to R_{14c} are optionally combined with each other to form a ring.

9. The heterocyclic compound of claim 8, wherein A₁₁ is a group represented by one of Formulae 3-11 to 3-16: 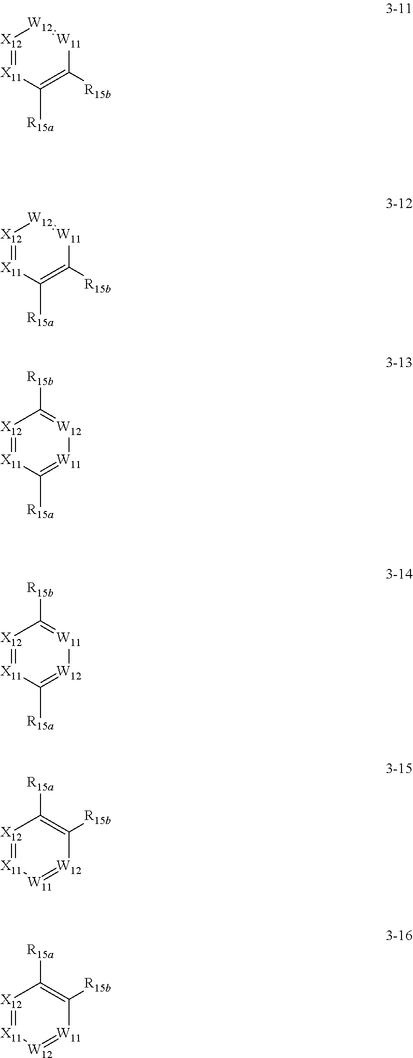
wherein, in Formulae 3-11 to 3-16,
X₁₁ to X₁₆ are each independently a nitrogen atom or a carbon atom,
W₁₁ and W₁₂ are each independently a nitrogen atom or a carbon atom, and
R₁₅ₐ and R_{15b} are each independently defined the same as R₁₁ in Formula 1.

10. The heterocyclic compound of claim 1, wherein the heterocyclic compound is a compound of Group C:

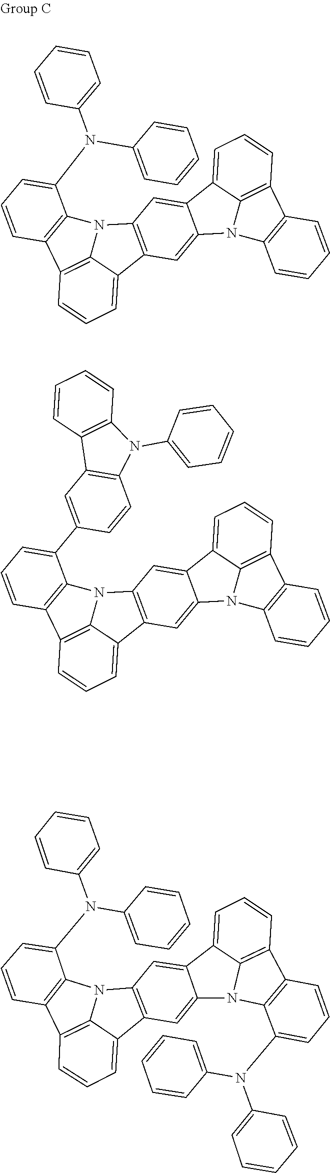 [SMILES: c1ccc(N(c2ccccc2)c2cccc3c4cccc5c6cc7c(cc6n(c23)c54)c2cccc3c4ccccc4n7c32)cc1 ; c1ccc(-n2c3ccccc3c3cc(-c4cccc5c6cccc7c8cc9c(cc8n(c45)c76)c4cccc5c6ccccc6n9c54)ccc32)cc1 ; c1ccc(N(c2ccccc2)c2cccc3c4cccc5c6cc7c(cc6n(c23)c54)c2cccc3c4cccc(N(c5ccccc5)c5ccccc5)c4n7c23)cc1]

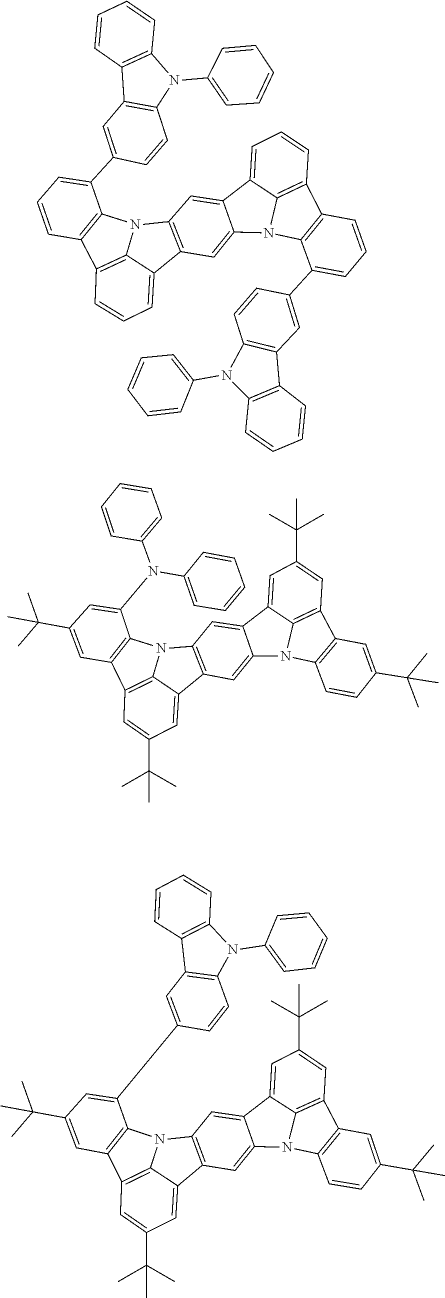

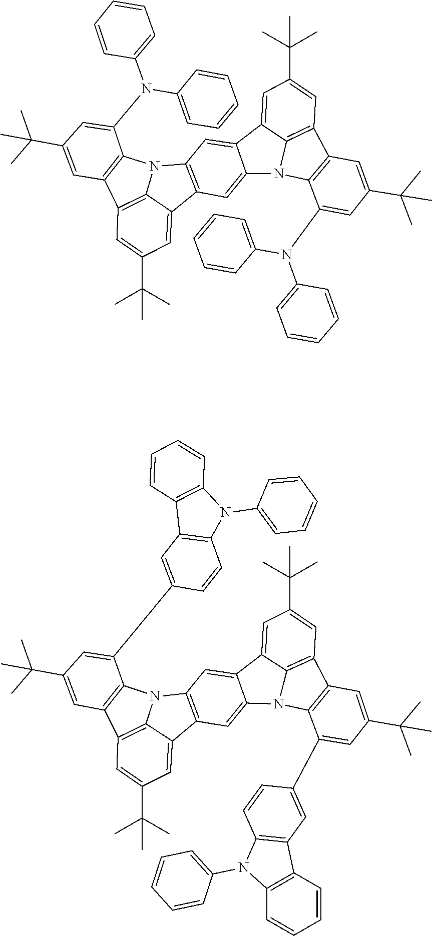

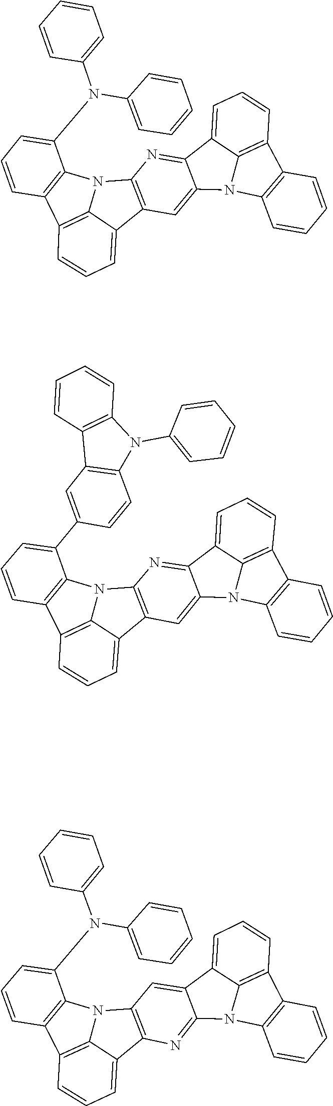 [SMILES: c1ccc(N(c2ccccc2)c2cccc3c4cccc5c6cc7c(nc6n(c23)c54)c2cccc3c4ccccc4n7c32)cc1 ; c1ccc(-n2c3ccccc3c3cc(-c4cccc5c6cccc7c8cc9c(nc8n(c45)c76)c4cccc5c6ccccc6n9c54)ccc32)cc1 ; c1ccc(N(c2ccccc2)c2cccc3c4cccc5c6nc7c(cc6n(c23)c45)c2cccc3c4ccccc4n7c32)cc1]

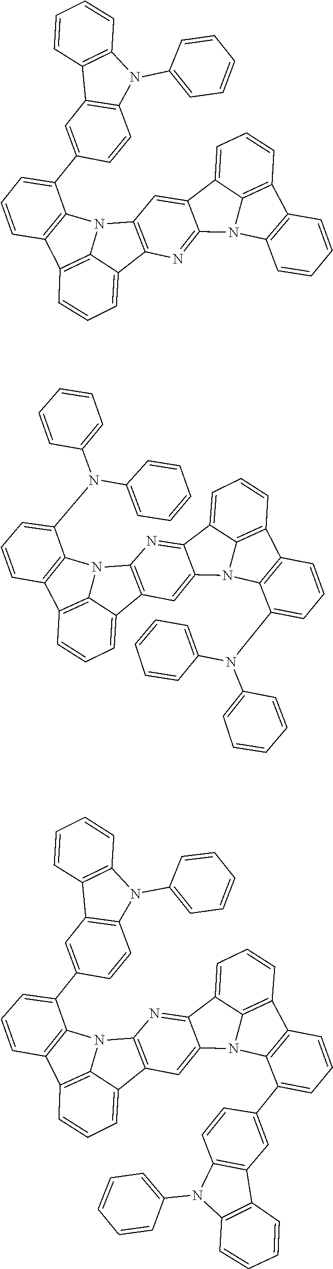 [SMILES: c1ccc(-n2c3ccccc3c3cc(-c4cccc5c6cccc7c8nc9c(cc8n(c45)c67)c4cccc5c6ccccc6n9c54)ccc32)cc1 ; c1ccc(N(c2ccccc2)c2cccc3c4cccc5c6nc7c(cc6n(c23)c45)c2cccc3c4cccc(N(c5ccccc5)c5ccccc5)c4n7c23)cc1 ; c1ccc(-n2c3ccccc3c3cc(-c4cccc5c6cccc7c8nc9c(cc8n(c45)c67)c4cccc5c6cccc(-c7ccc8c(c7)c7ccccc7n8-c7ccccc7)c6n9c45)ccc32)cc1]

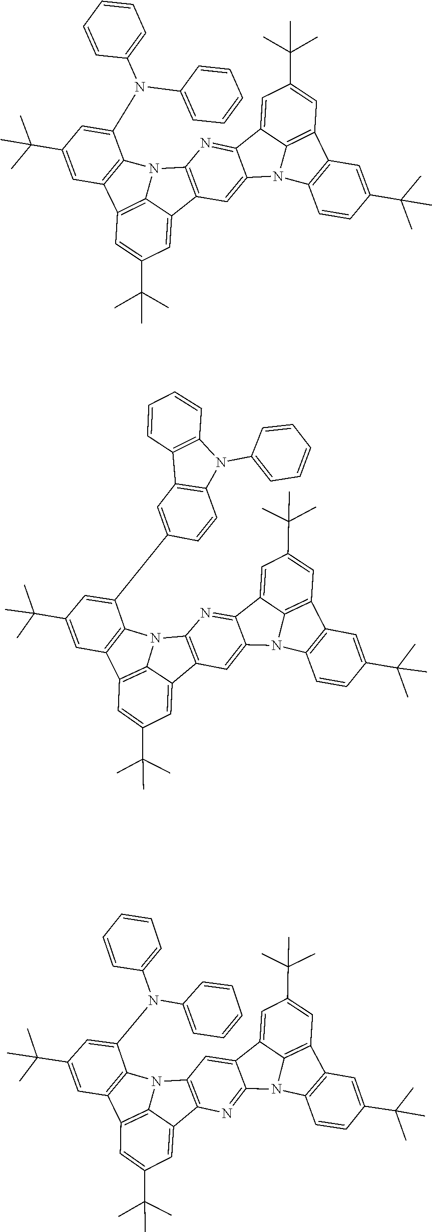

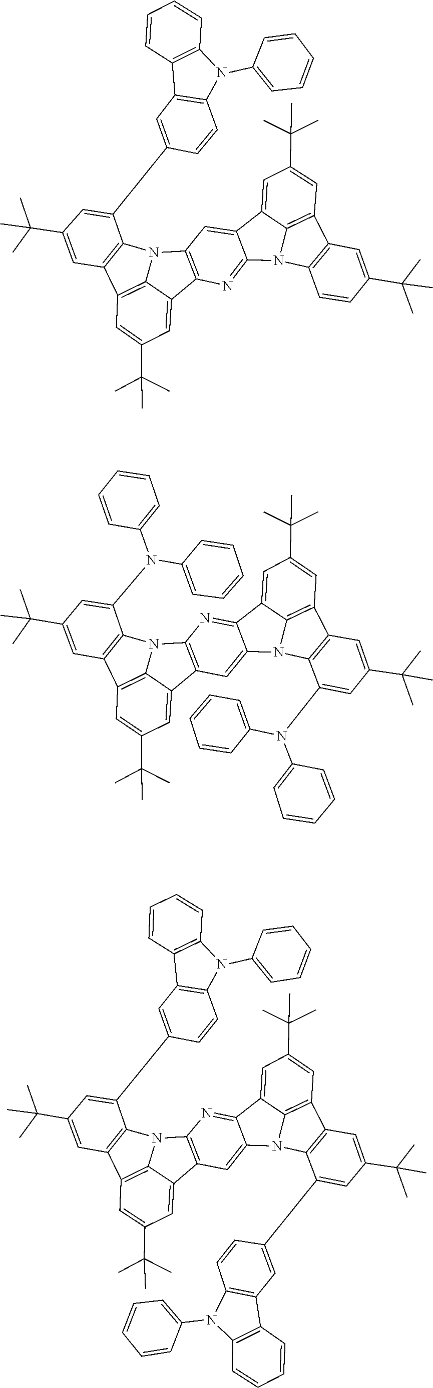

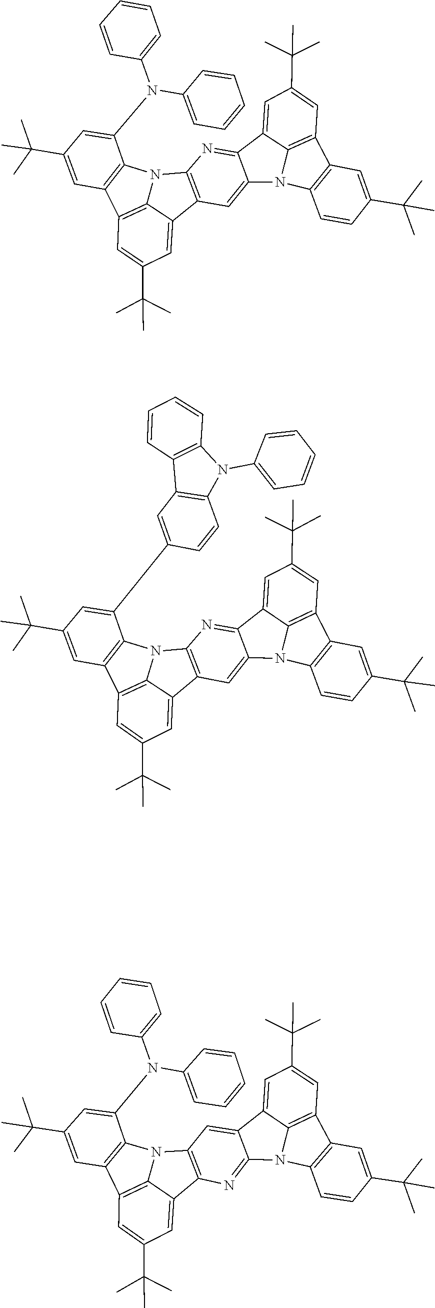

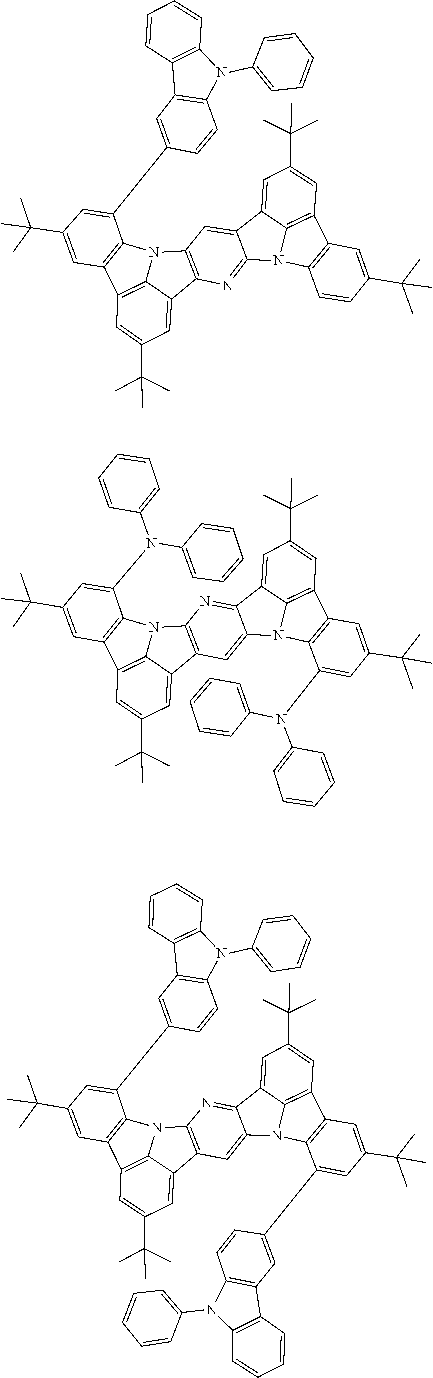

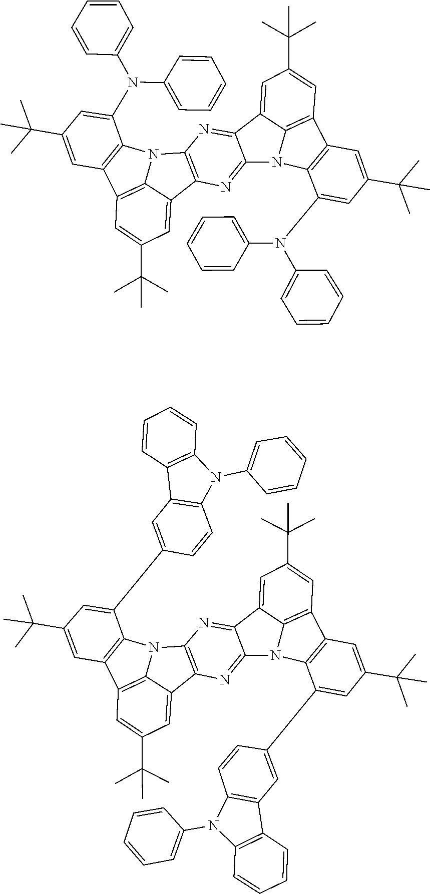

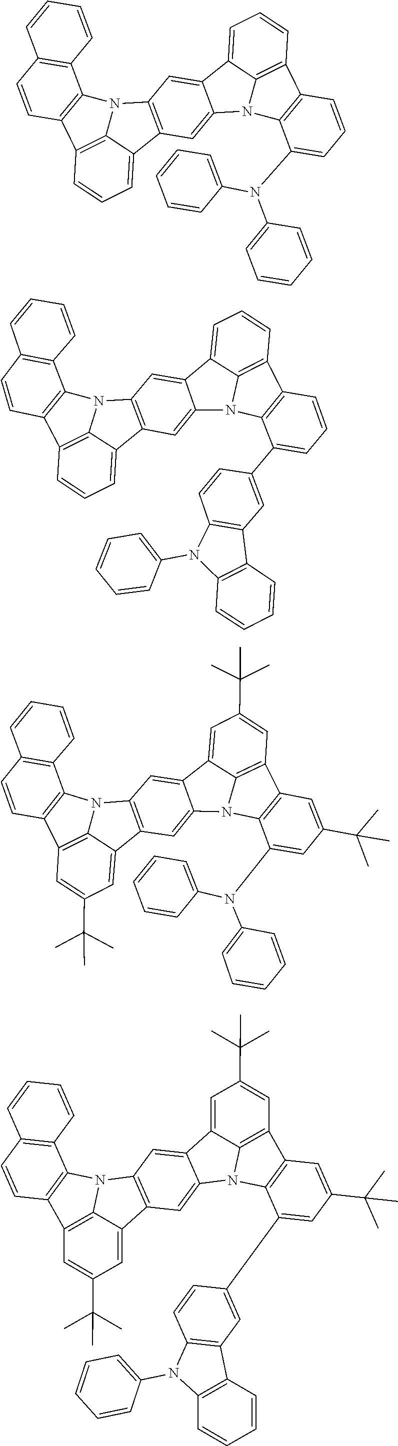

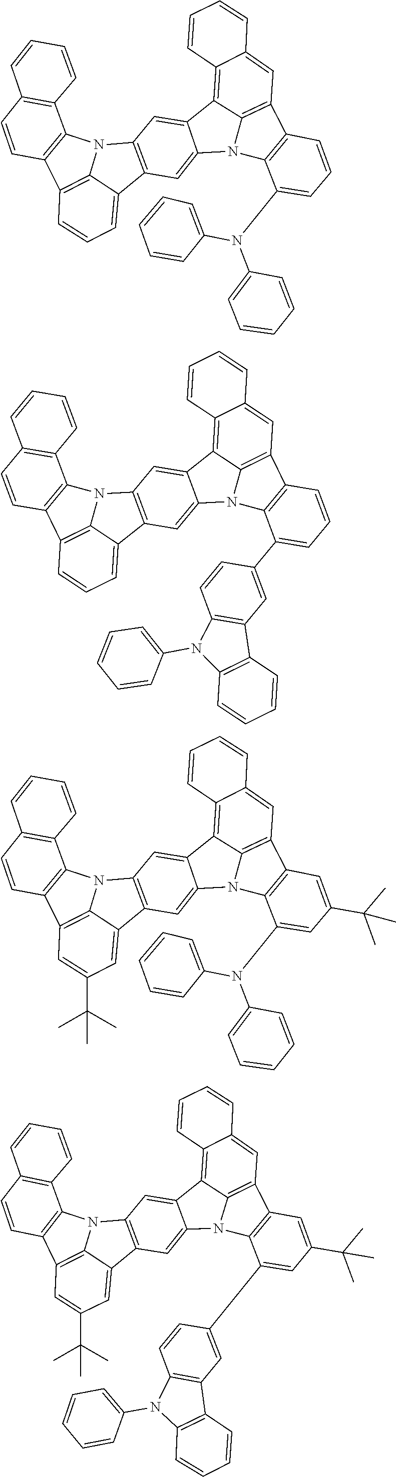

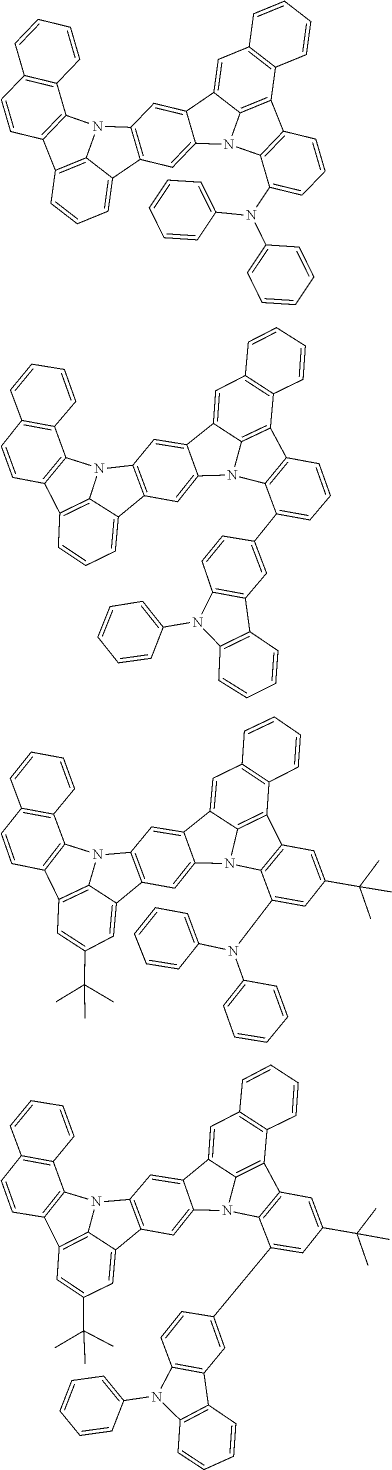

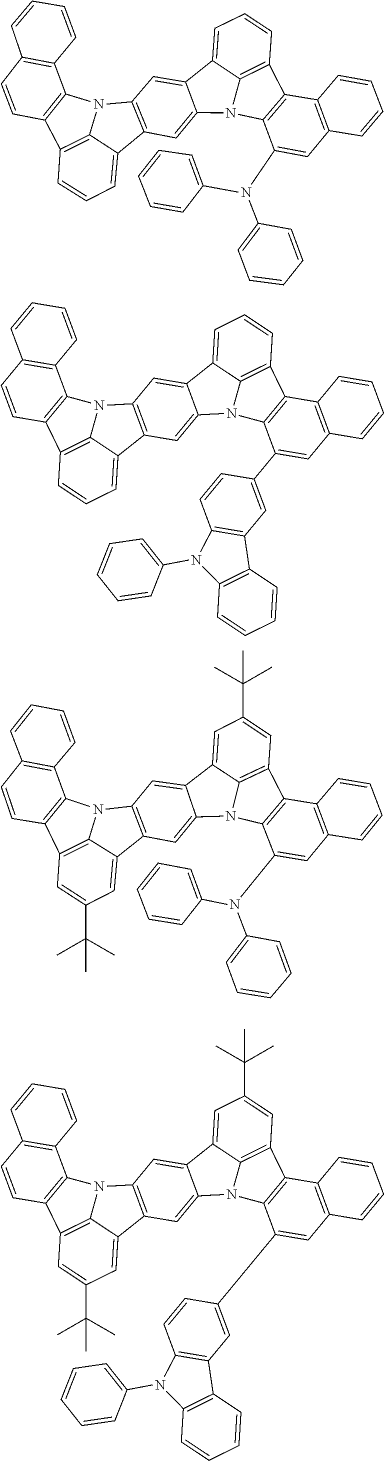

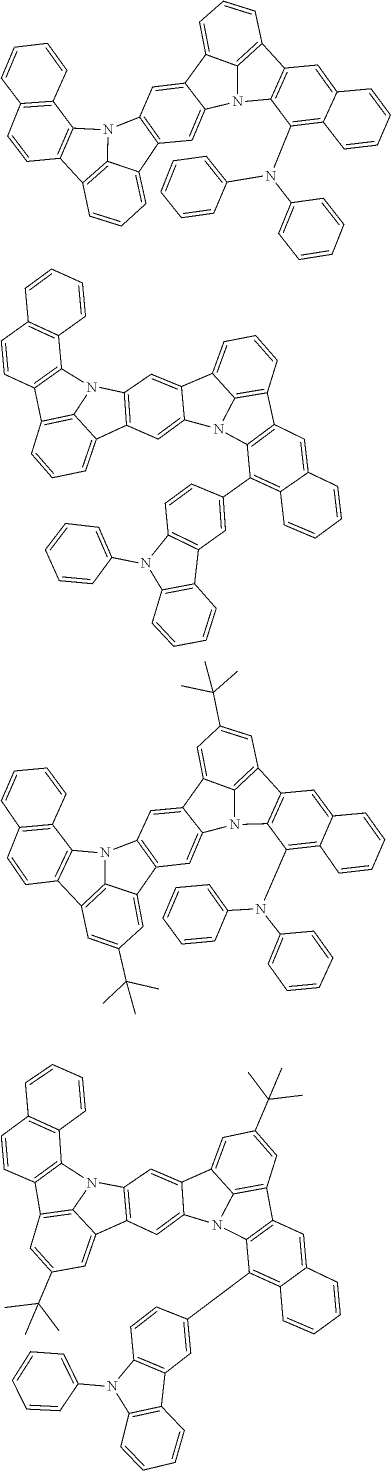

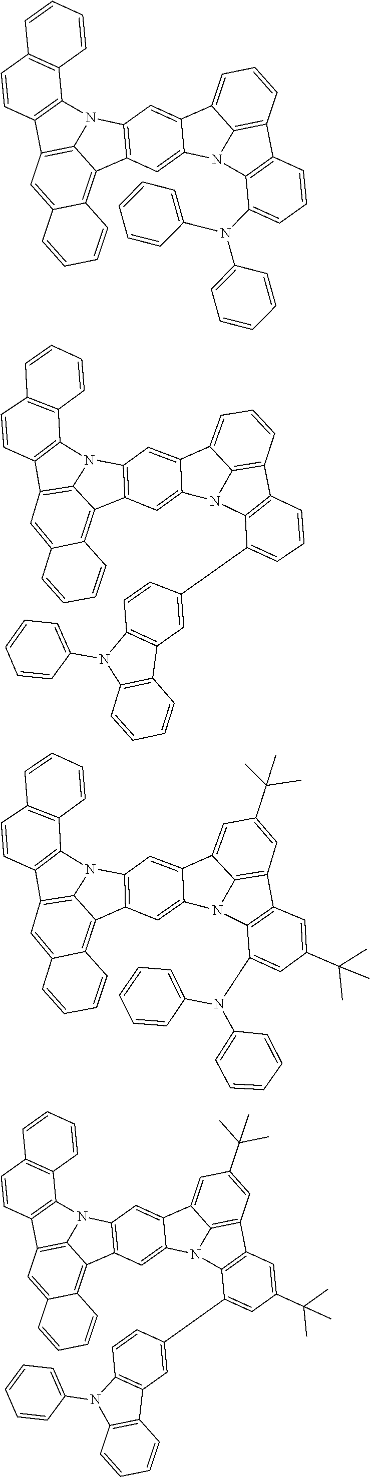

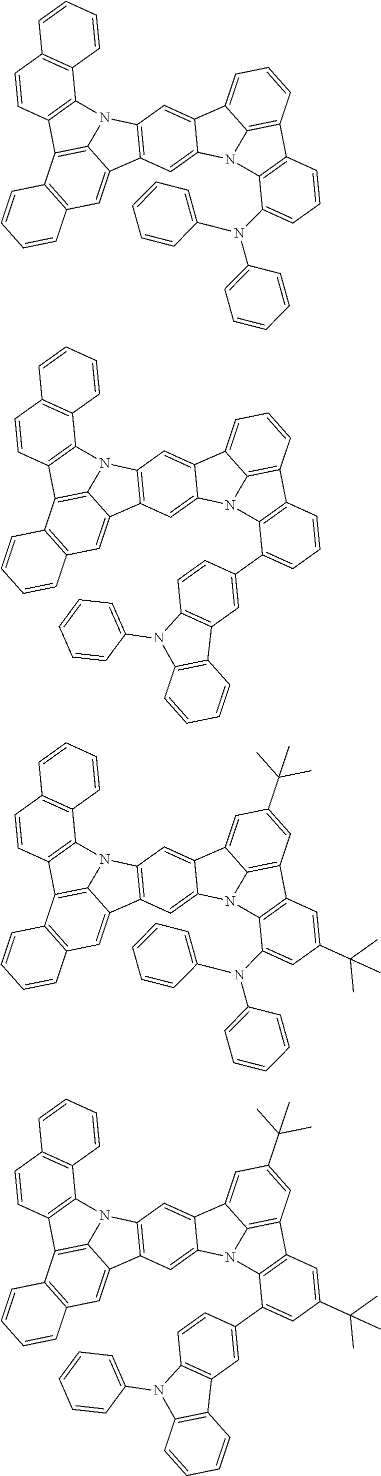

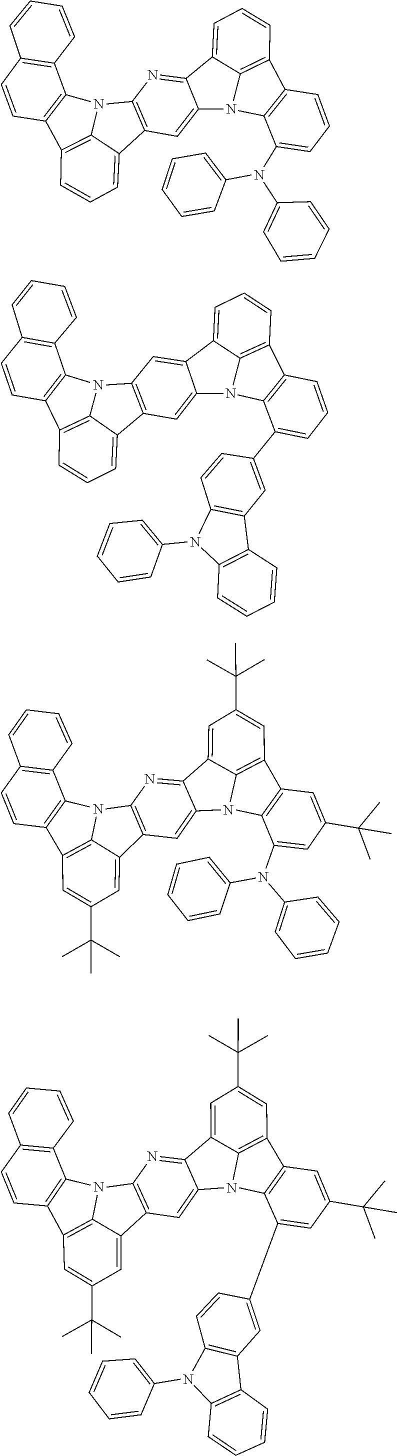

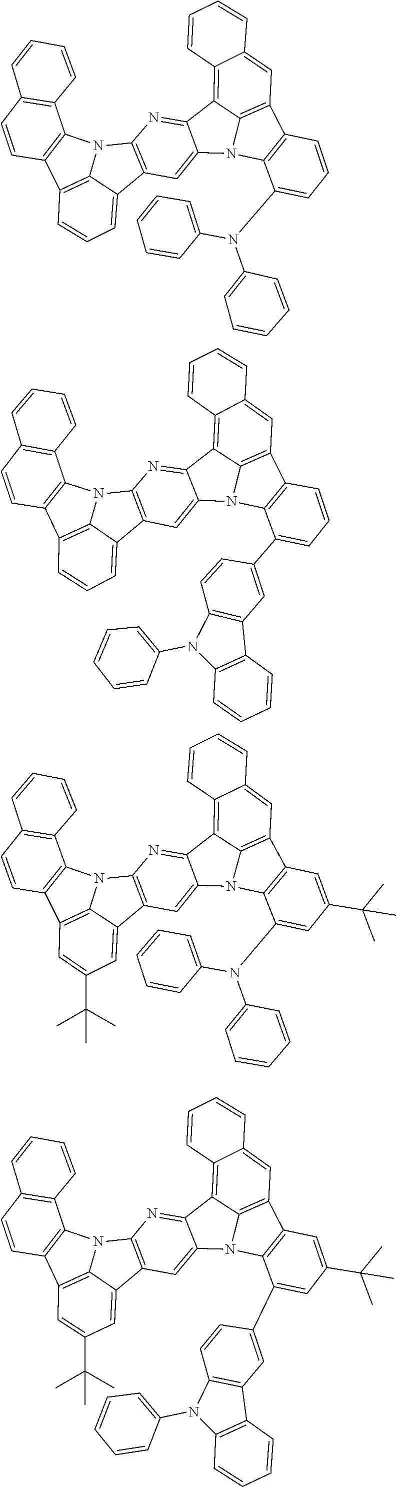

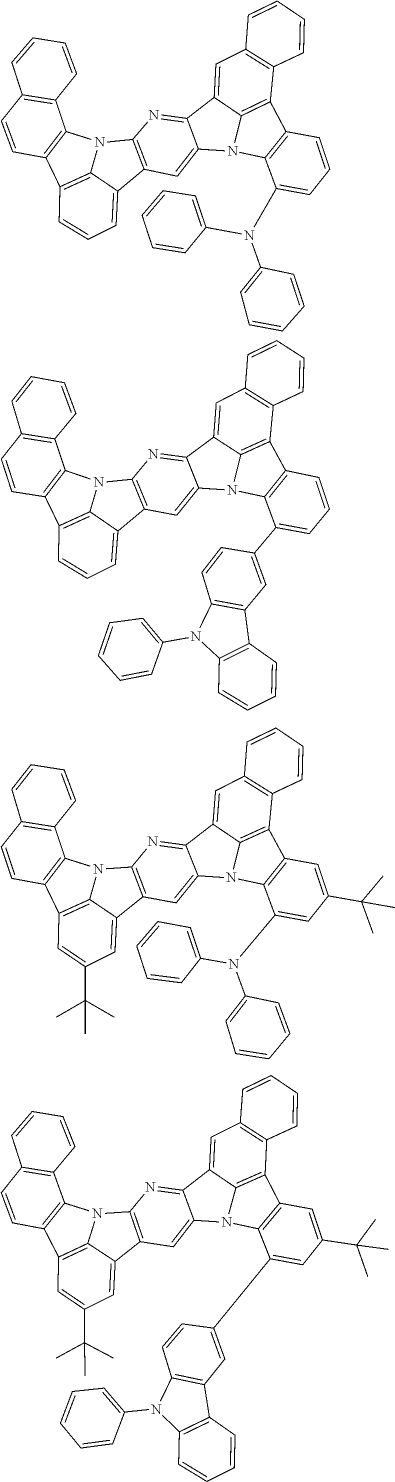

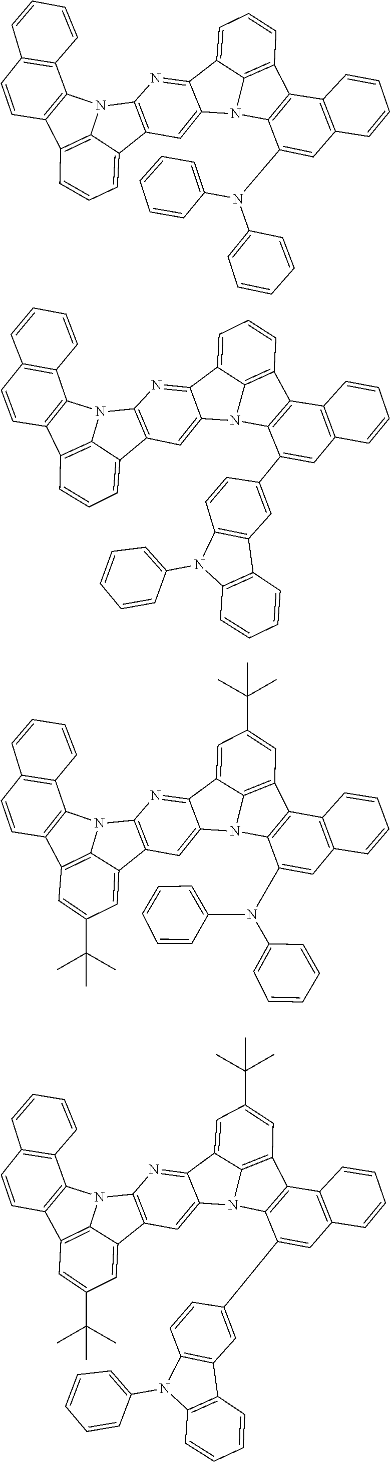

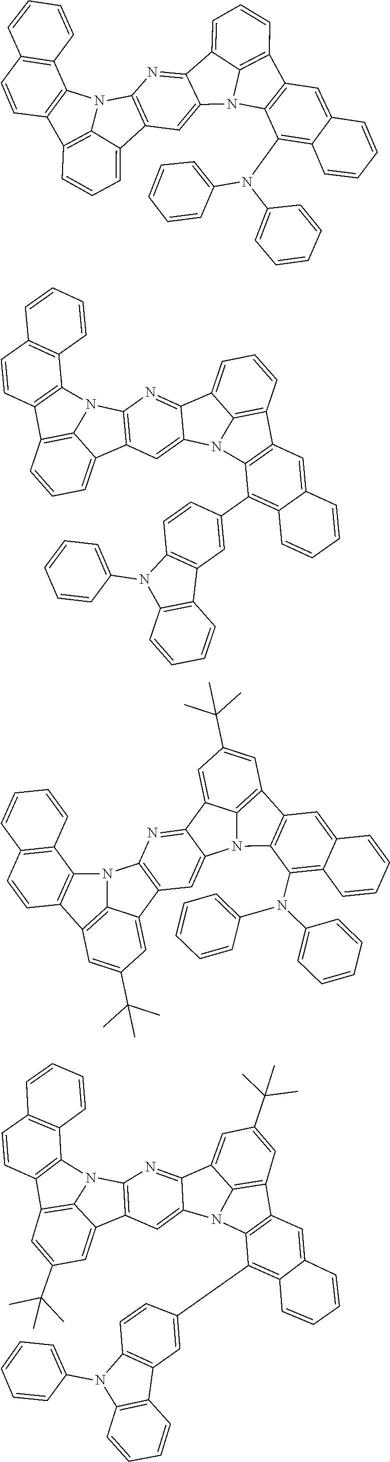

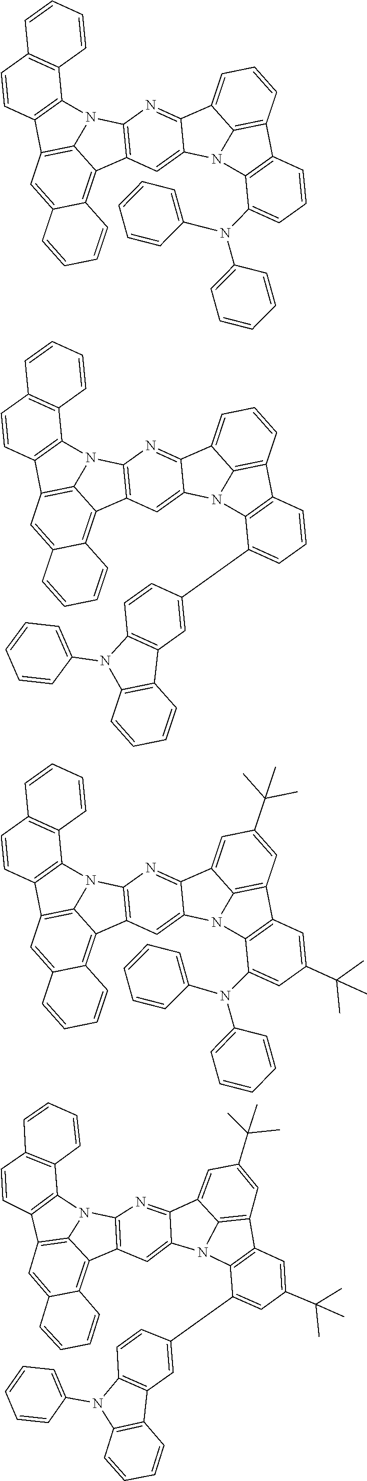

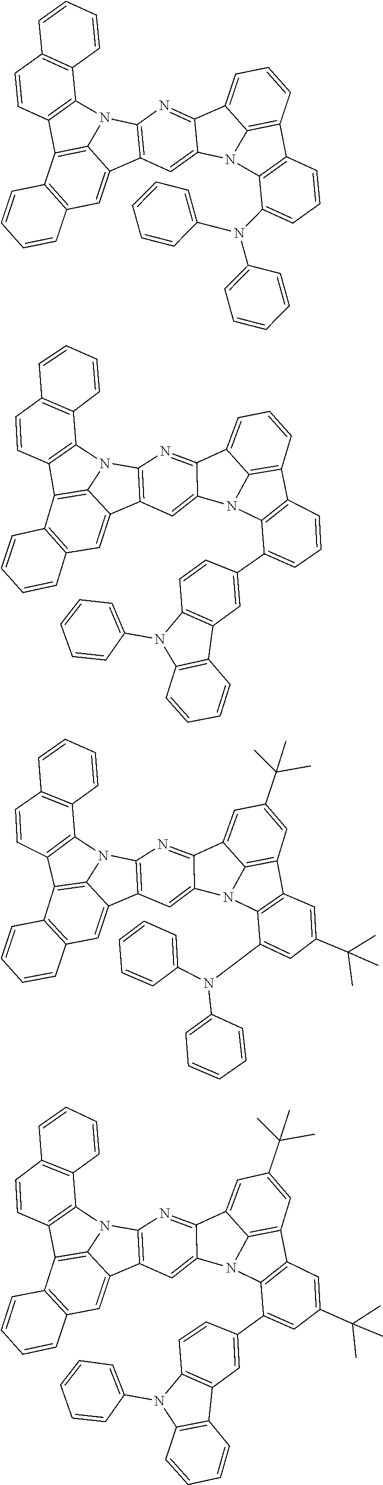 [SMILES: c1ccc(N(c2ccccc2)c2cccc3c4cccc5c6nc7c(cc6n(c23)c45)c2cc3ccccc3c3c4ccc5ccccc5c4n7c23)cc1 ; c1ccc(-n2c3ccccc3c3cc(-c4cccc5c6cccc7c8nc9c(cc8n(c45)c67)c4cc5ccccc5c5c6ccc7ccccc7c6n9c45)ccc32)cc1 ; CC(C)(C)c1cc(N(c2ccccc2)c2ccccc2)c2c(c1)c1cc(C(C)(C)C)cc3c4nc5c(cc4n2c13)c1cc2ccccc2c2c3ccc4ccccc4c3n5c12 ; CC(C)(C)c1cc(-c2ccc3c(c2)c2ccccc2n3-c2ccccc2)c2c(c1)c1cc(C(C)(C)C)cc3c4nc5c(cc4n2c13)c1cc2ccccc2c2c3ccc4ccccc4c3n5c12]

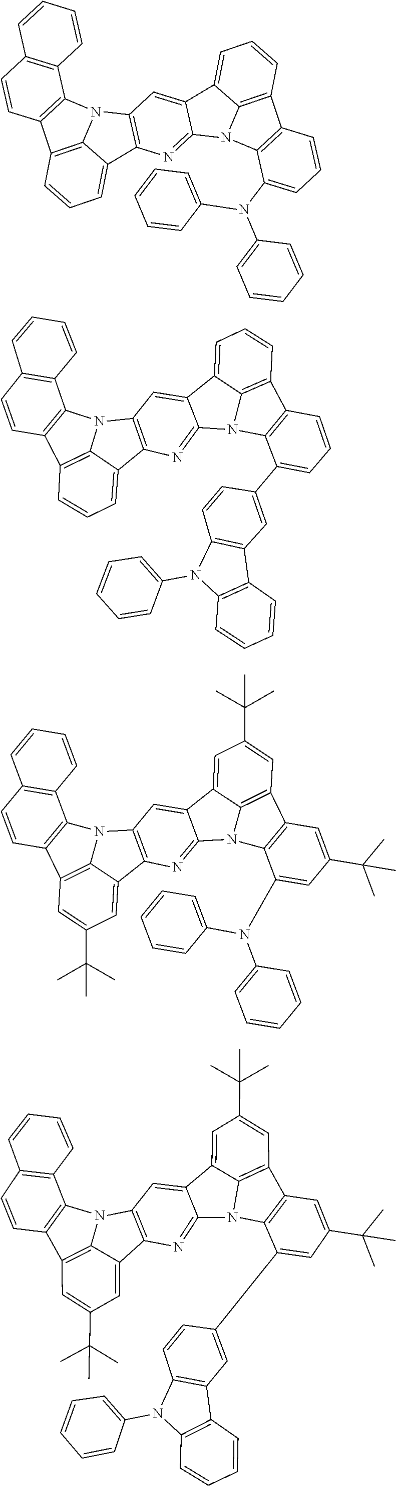

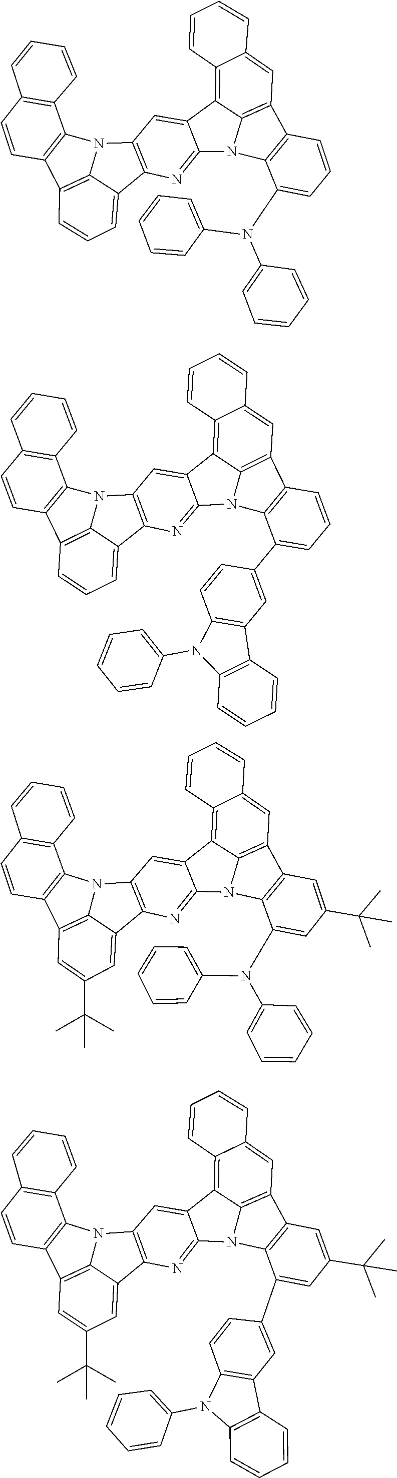

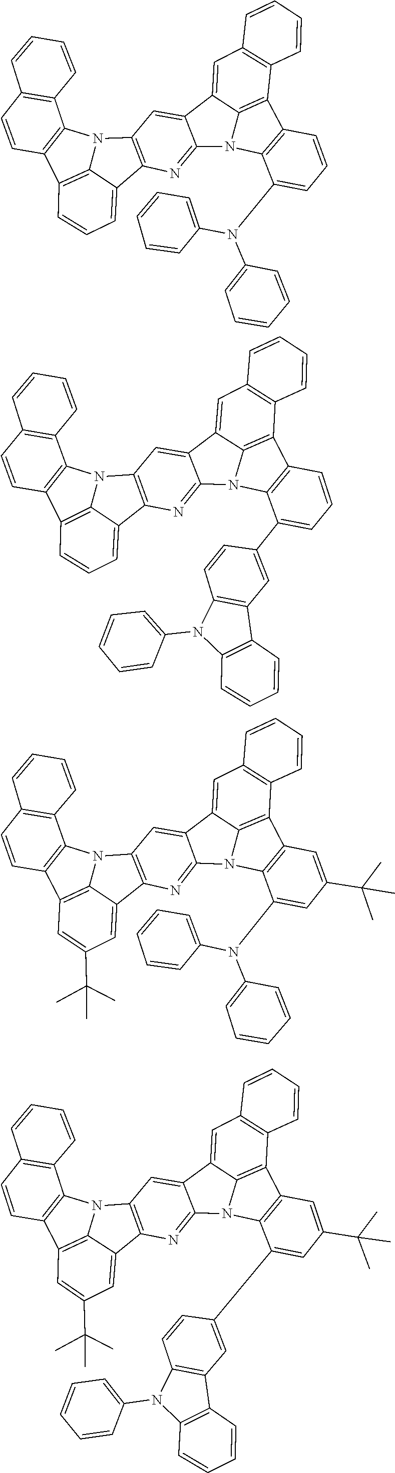

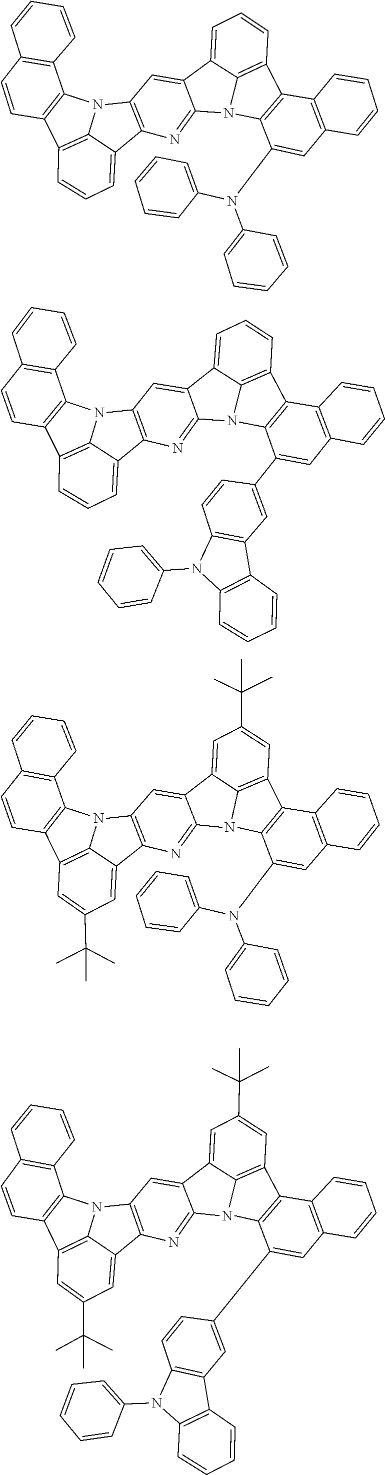

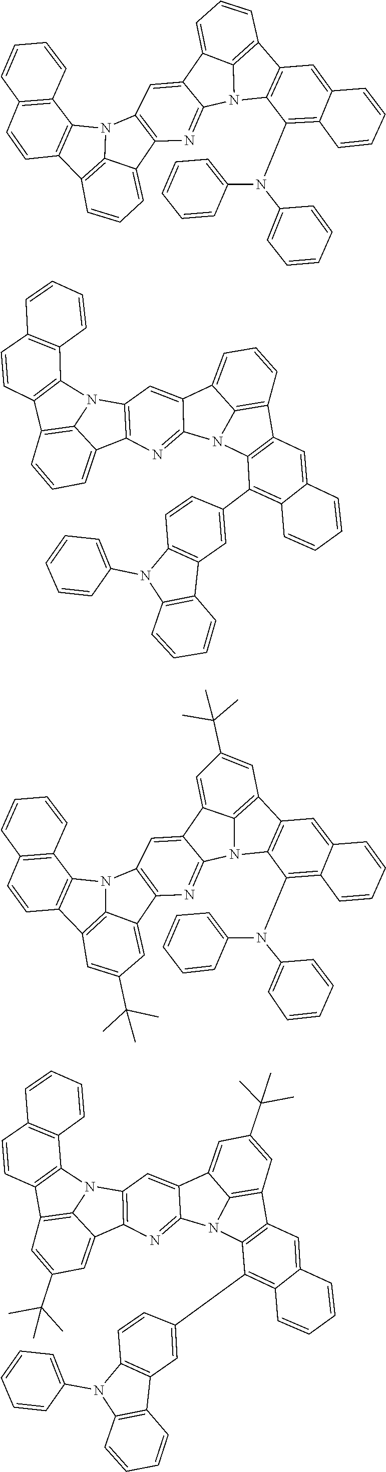

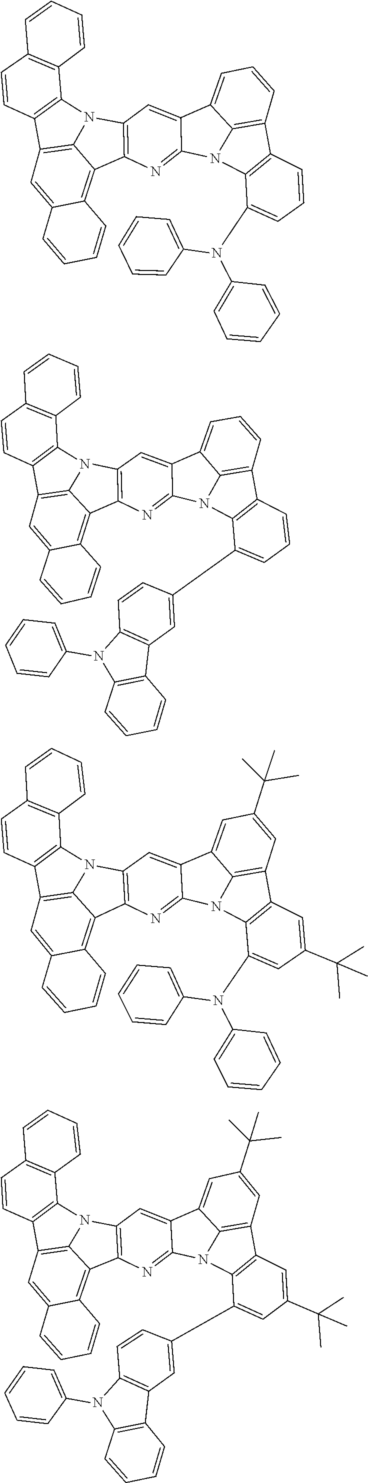

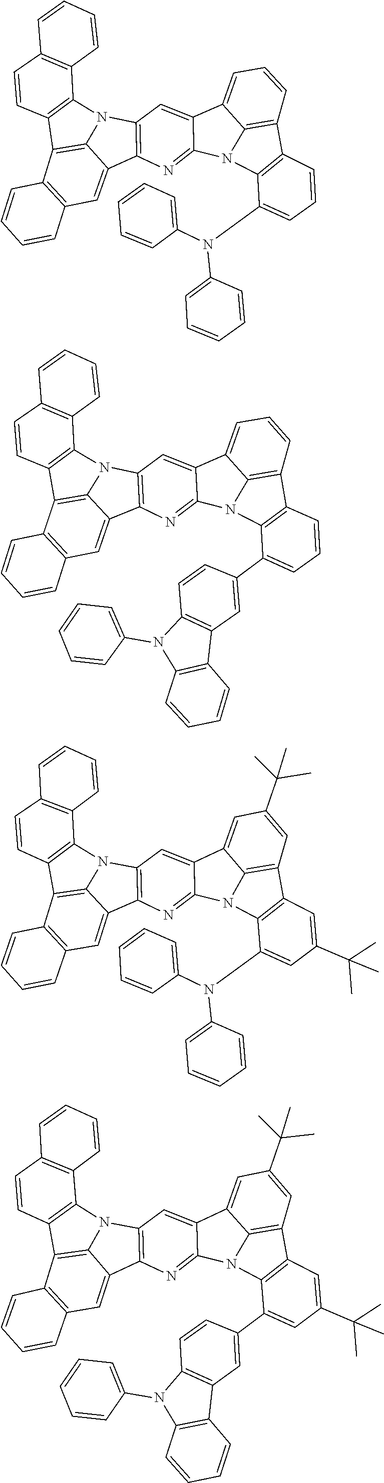 [SMILES: c1ccc(N(c2ccccc2)c2cccc3c4cccc5c6cc7c(nc6n(c23)c54)c2cc3ccccc3c3c4ccc5ccccc5c4n7c23)cc1 ; c1ccc(-n2c3ccccc3c3cc(-c4cccc5c6cccc7c8cc9c(nc8n(c45)c76)c4cc5ccccc5c5c6ccc7ccccc7c6n9c45)ccc32)cc1 ; CC(C)(C)c1cc(N(c2ccccc2)c2ccccc2)c2c(c1)c1cc(C(C)(C)C)cc3c4cc5c(nc4n2c31)c1cc2ccccc2c2c3ccc4ccccc4c3n5c12 ; CC(C)(C)c1cc(-c2ccc3c(c2)c2ccccc2n3-c2ccccc2)c2c(c1)c1cc(C(C)(C)C)cc3c4cc5c(nc4n2c31)c1cc2ccccc2c2c3ccc4ccccc4c3n5c12]

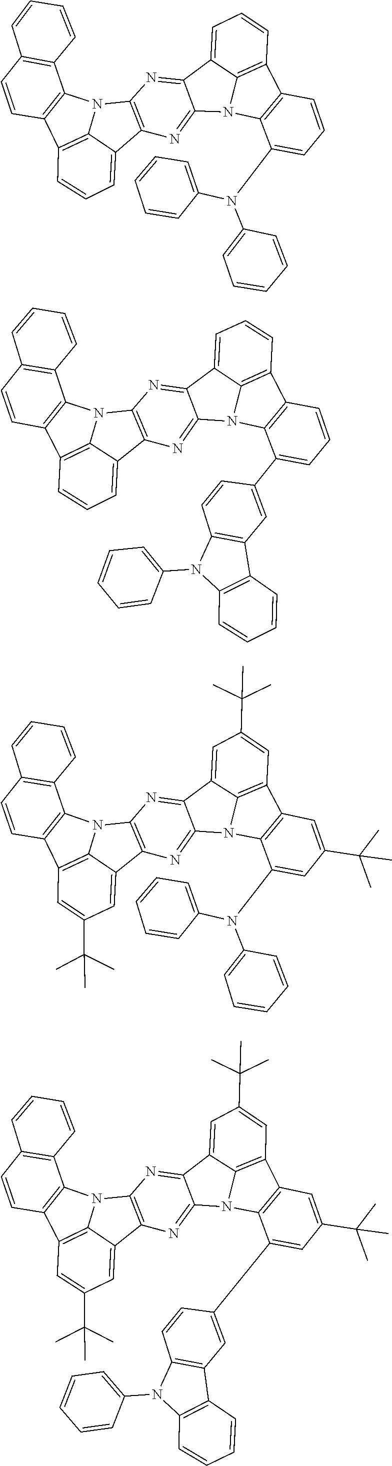

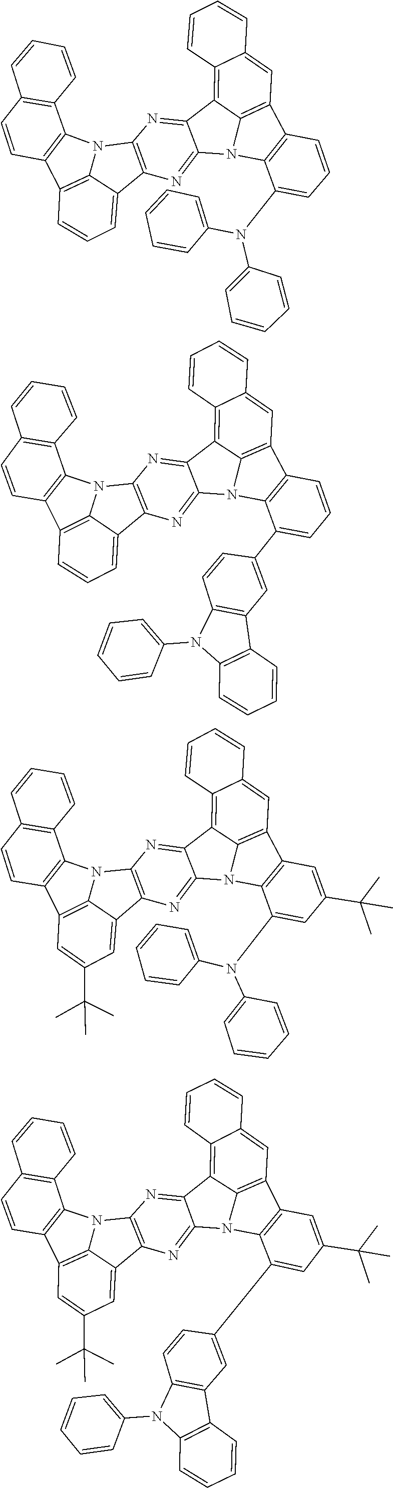

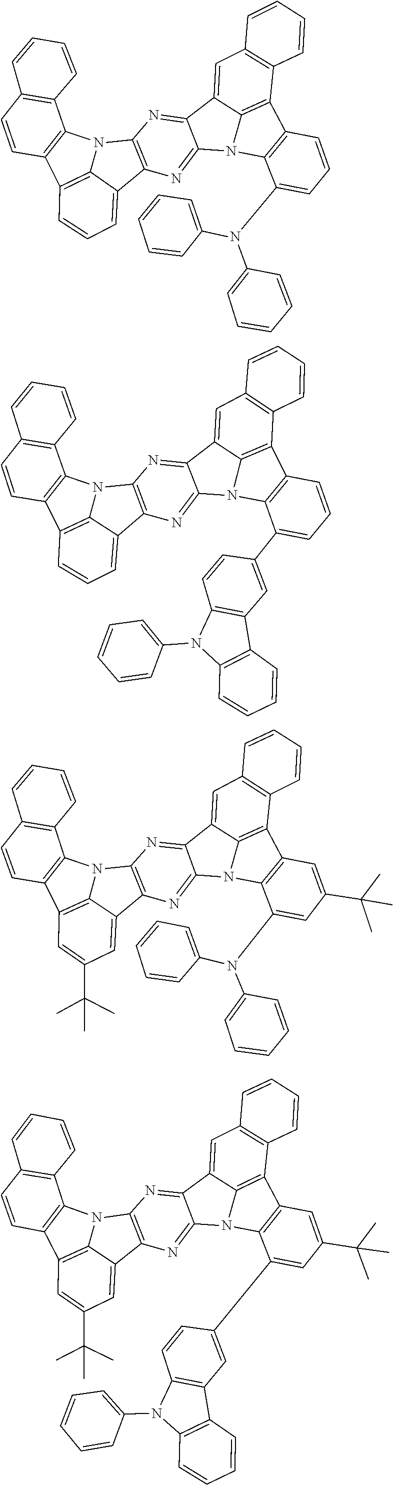

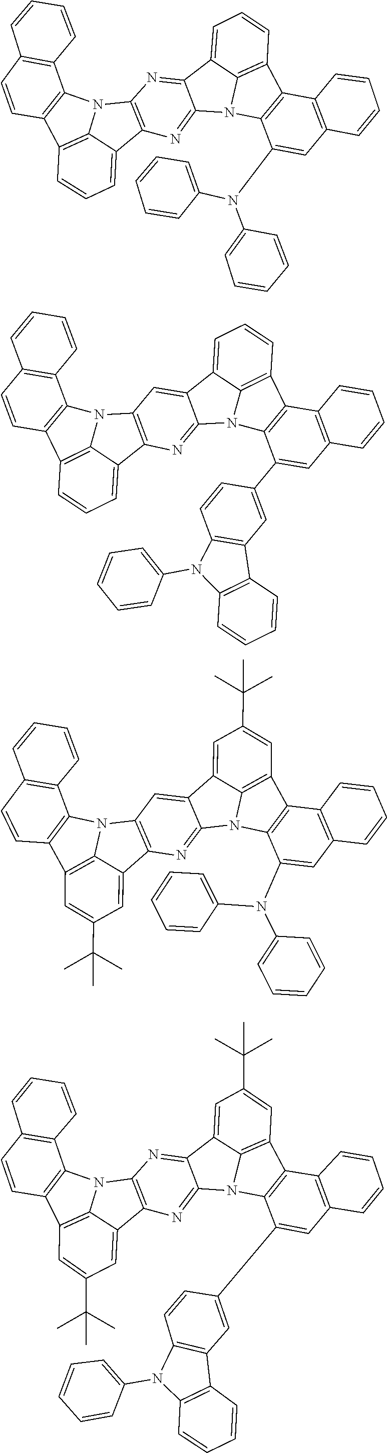

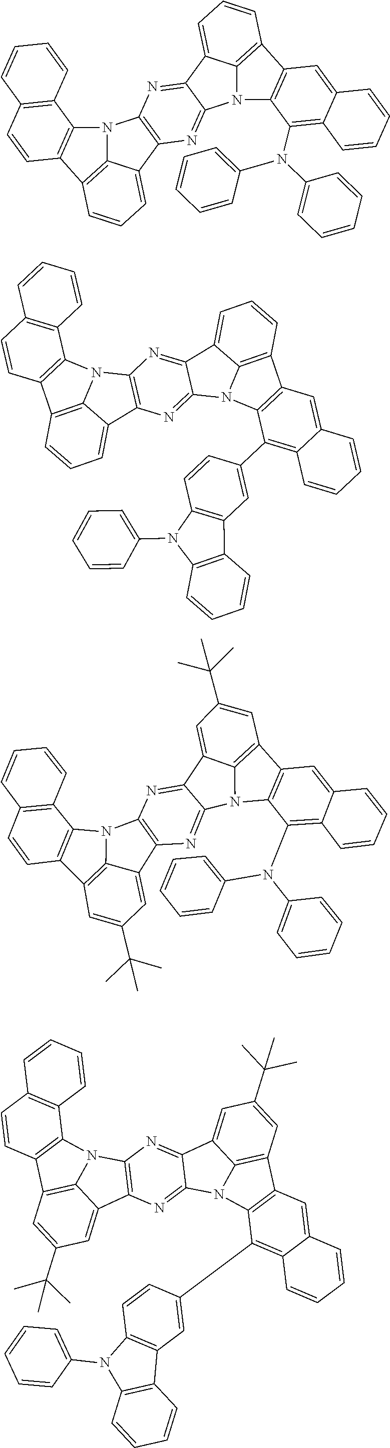

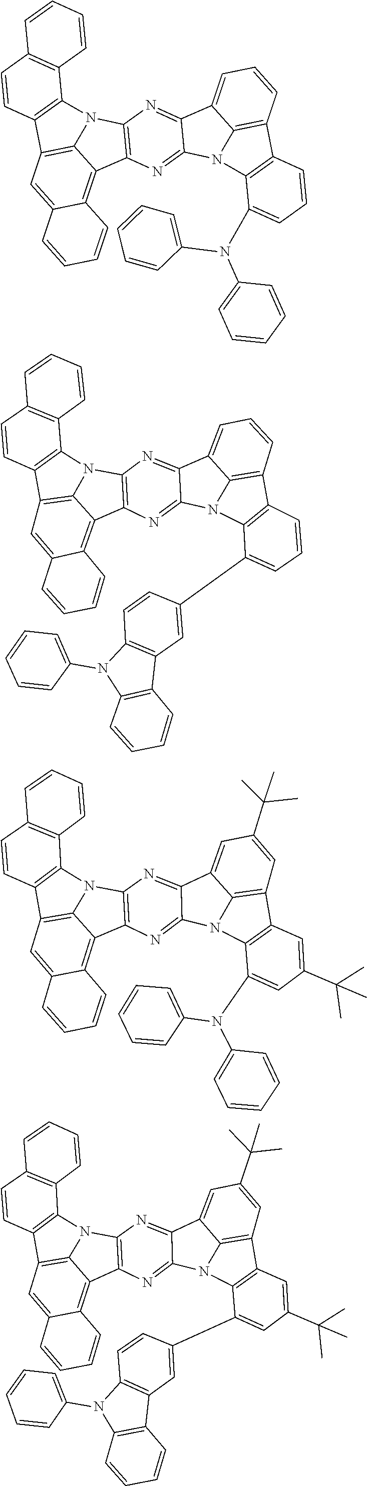

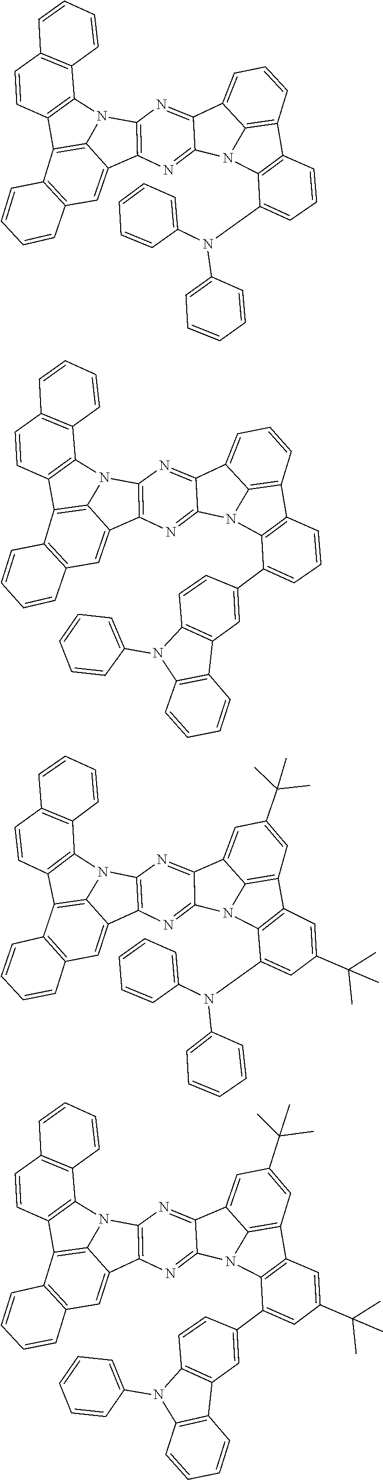

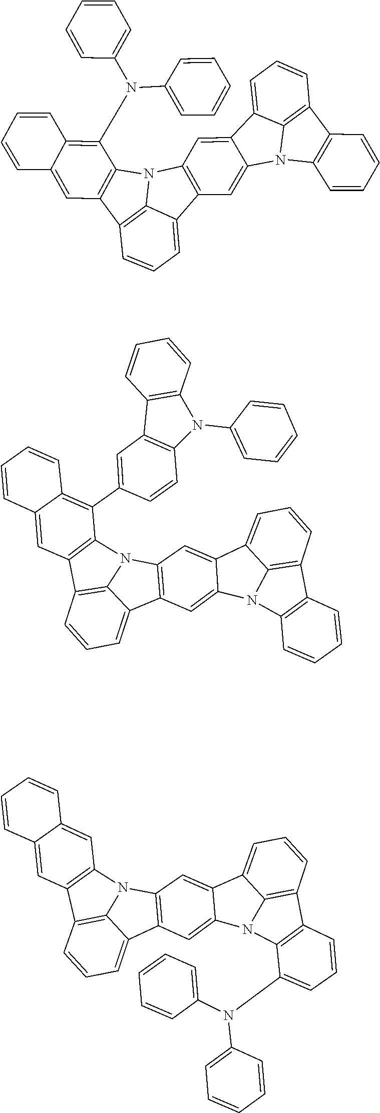 [SMILES: c1ccc(N(c2ccccc2)c2c3ccccc3cc3c4cccc5c6cc7c(cc6n(c23)c54)c2cccc3c4ccccc4n7c32)cc1 ; c1ccc(-n2c3ccccc3c3cc(-c4c5ccccc5cc5c6cccc7c8cc9c(cc8n(c45)c76)c4cccc5c6ccccc6n9c54)ccc32)cc1 ; c1ccc(N(c2ccccc2)c2cccc3c4cccc5c6cc7c(cc6n(c23)c54)c2cccc3c4cc5ccccc5cc4n7c32)cc1]

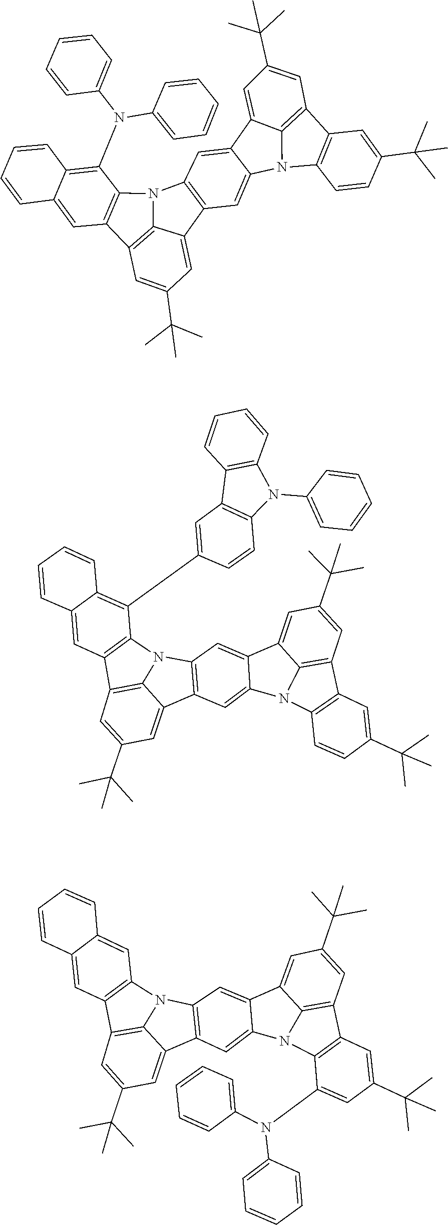 [SMILES: CC(C)(C)c1ccc2c(c1)c1cc(C(C)(C)C)cc3c4cc5c(cc4n2c13)c1cc(C(C)(C)C)cc2c3cc4ccccc4c(N(c4ccccc4)c4ccccc4)c3n5c12 ; CC(C)(C)c1ccc2c(c1)c1cc(C(C)(C)C)cc3c4cc5c(cc4n2c13)c1cc(C(C)(C)C)cc2c3cc4ccccc4c(-c4ccc6c(c4)c4ccccc4n6-c4ccccc4)c3n5c12 ; CC(C)(C)c1cc(N(c2ccccc2)c2ccccc2)c2c(c1)c1cc(C(C)(C)C)cc3c4cc5c(cc4n2c31)c1cc(C(C)(C)C)cc2c3cc4ccccc4cc3n5c21]

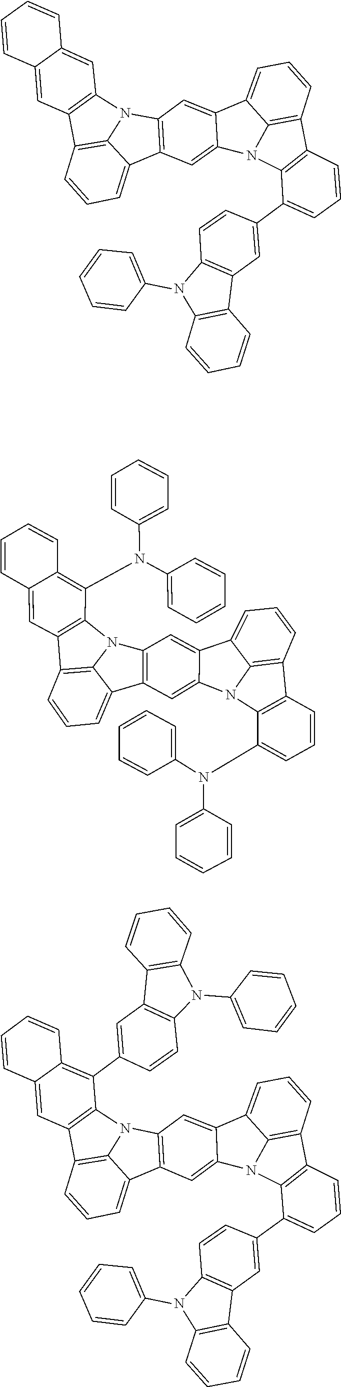 [SMILES: c1ccc(-n2c3ccccc3c3cc(-c4cccc5c6cccc7c8cc9c(cc8n(c45)c76)c4cccc5c6cc7ccccc7cc6n9c54)ccc32)cc1 ; c1ccc(N(c2ccccc2)c2cccc3c4cccc5c6cc7c(cc6n(c23)c54)c2cccc3c4cc5ccccc5c(N(c5ccccc5)c5ccccc5)c4n7c23)cc1 ; c1ccc(-n2c3ccccc3c3cc(-c4cccc5c6cccc7c8cc9c(cc8n(c45)c76)c4cccc5c6cc7ccccc7c(-c7ccc8c(c7)c7ccccc7n8-c7ccccc7)c6n9c45)ccc32)cc1]

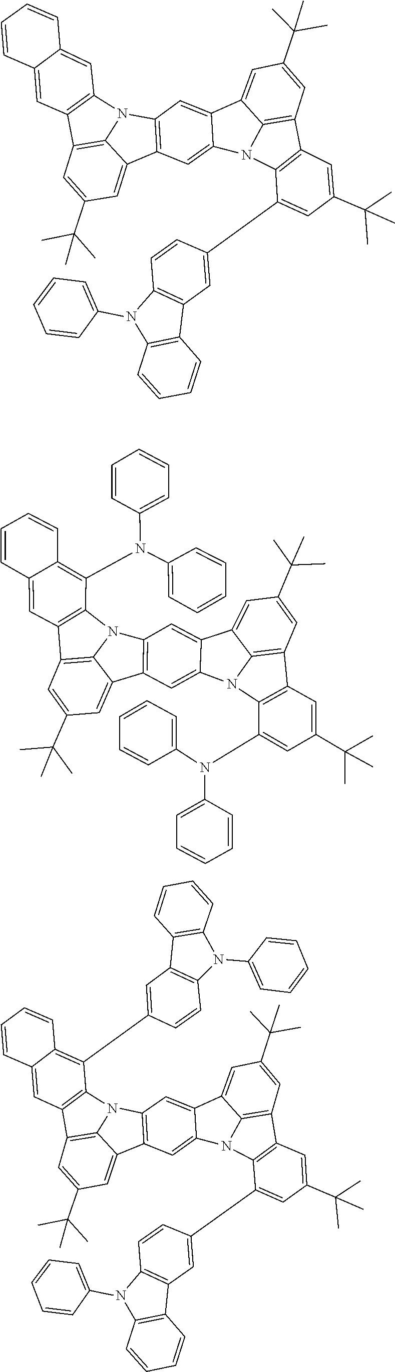

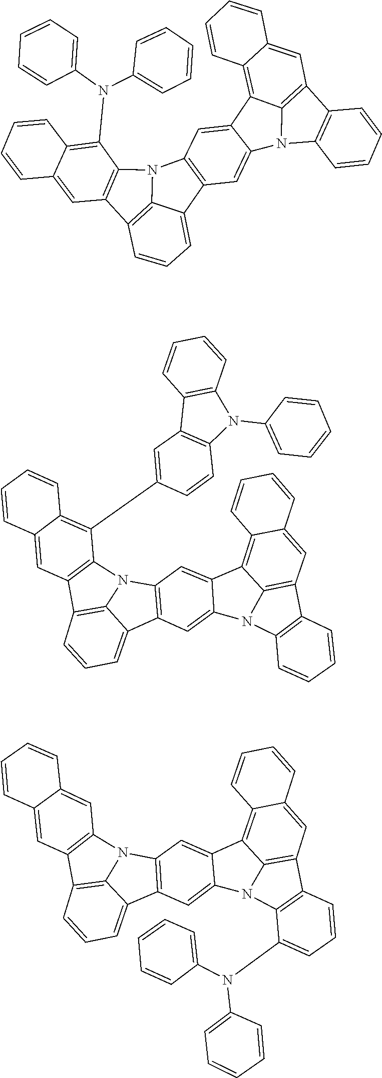 [SMILES: c1ccc(N(c2ccccc2)c2c3ccccc3cc3c4cccc5c6cc7c(cc6n(c23)c54)c2c3ccccc3cc3c4ccccc4n7c32)cc1 ; c1ccc(-n2c3ccccc3c3cc(-c4c5ccccc5cc5c6cccc7c8cc9c(cc8n(c45)c76)c4c5ccccc5cc5c6ccccc6n9c54)ccc32)cc1 ; c1ccc(N(c2ccccc2)c2cccc3c4cc5ccccc5c5c6cc7c(cc6n(c23)c45)c2cccc3c4cc5ccccc5cc4n7c32)cc1]

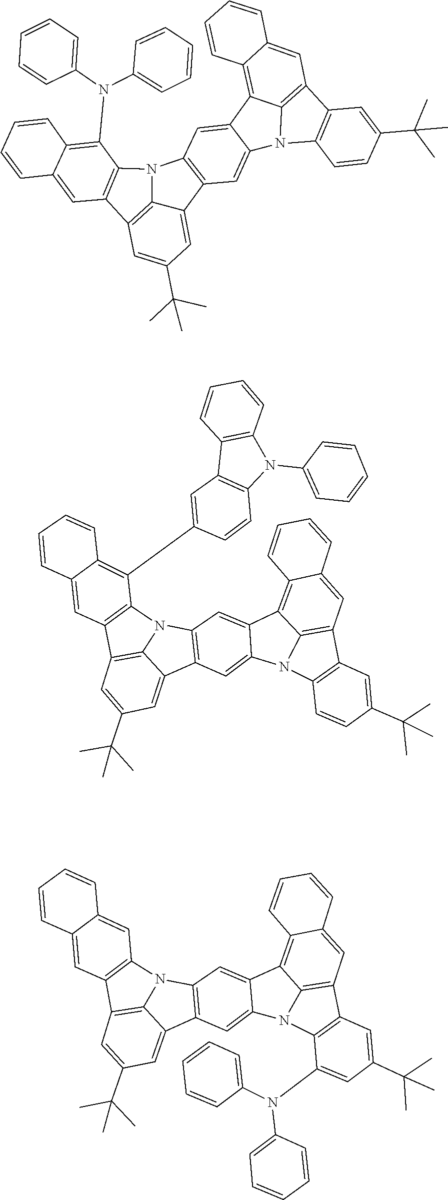 [SMILES: CC(C)(C)c1ccc2c(c1)c1cc3ccccc3c3c4cc5c(cc4n2c13)c1cc(C(C)(C)C)cc2c3cc4ccccc4c(N(c4ccccc4)c4ccccc4)c3n5c12 ; CC(C)(C)c1ccc2c(c1)c1cc3ccccc3c3c4cc5c(cc4n2c13)c1cc(C(C)(C)C)cc2c3cc4ccccc4c(-c4ccc6c(c4)c4ccccc4n6-c4ccccc4)c3n5c12 ; CC(C)(C)c1cc(N(c2ccccc2)c2ccccc2)c2c(c1)c1cc3ccccc3c3c4cc5c(cc4n2c13)c1cc(C(C)(C)C)cc2c3cc4ccccc4cc3n5c21]

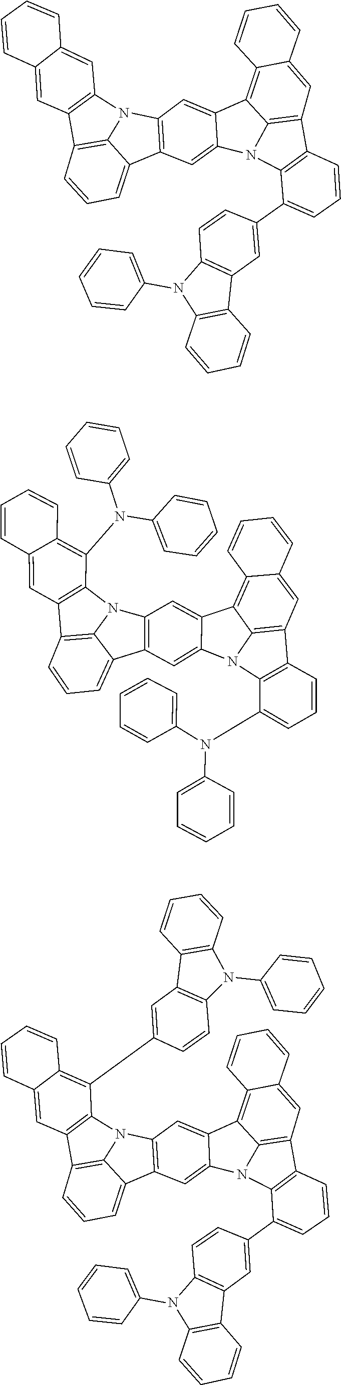 [SMILES: c1ccc(-n2c3ccccc3c3cc(-c4cccc5c6cc7ccccc7c7c8cc9c(cc8n(c45)c67)c4cccc5c6cc7ccccc7cc6n9c54)ccc32)cc1 ; c1ccc(N(c2ccccc2)c2c3ccccc3cc3c4cccc5c6cc7c(cc6n(c23)c54)c2c3ccccc3cc3c4cccc(N(c5ccccc5)c5ccccc5)c4n7c32)cc1 ; c1ccc(-n2c3ccccc3c3cc(-c4c5ccccc5cc5c6cccc7c8cc9c(cc8n(c45)c76)c4c5ccccc5cc5c6cccc(-c7ccc8c(c7)c7ccccc7n8-c7ccccc7)c6n9c54)ccc32)cc1]

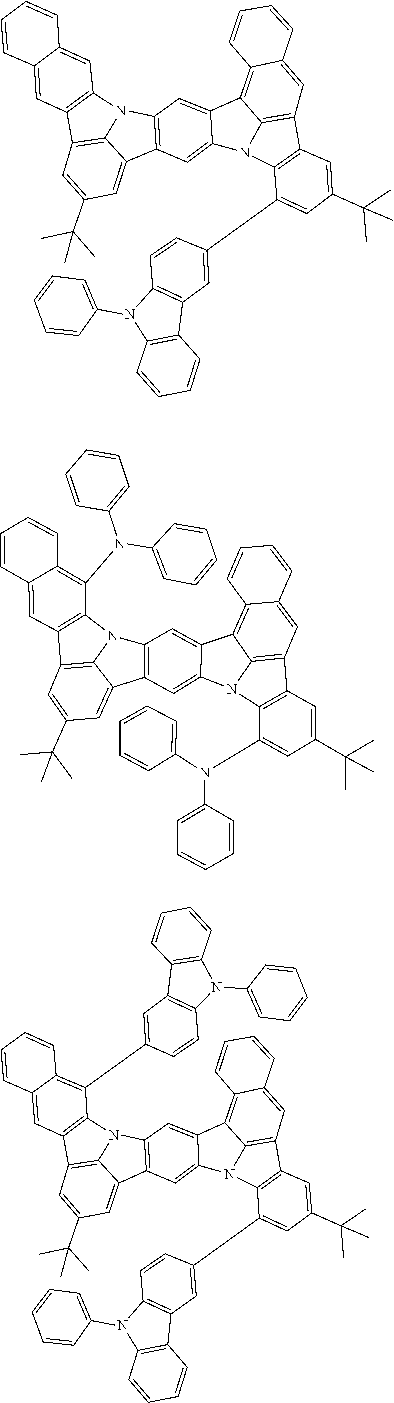

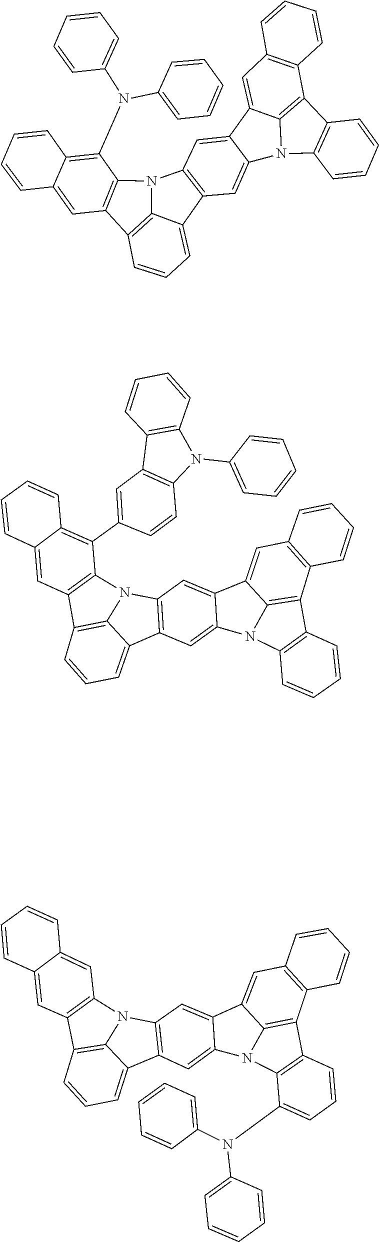 [SMILES: c1ccc(N(c2ccccc2)c2c3ccccc3cc3c4cccc5c6cc7c(cc6n(c23)c54)c2cc3ccccc3c3c4ccccc4n7c23)cc1 ; c1ccc(-n2c3ccccc3c3cc(-c4c5ccccc5cc5c6cccc7c8cc9c(cc8n(c45)c76)c4cc5ccccc5c5c6ccccc6n9c45)ccc32)cc1 ; c1ccc(N(c2ccccc2)c2cccc3c4c5ccccc5cc5c6cc7c(cc6n(c23)c54)c2cccc3c4cc5ccccc5cc4n7c32)cc1]

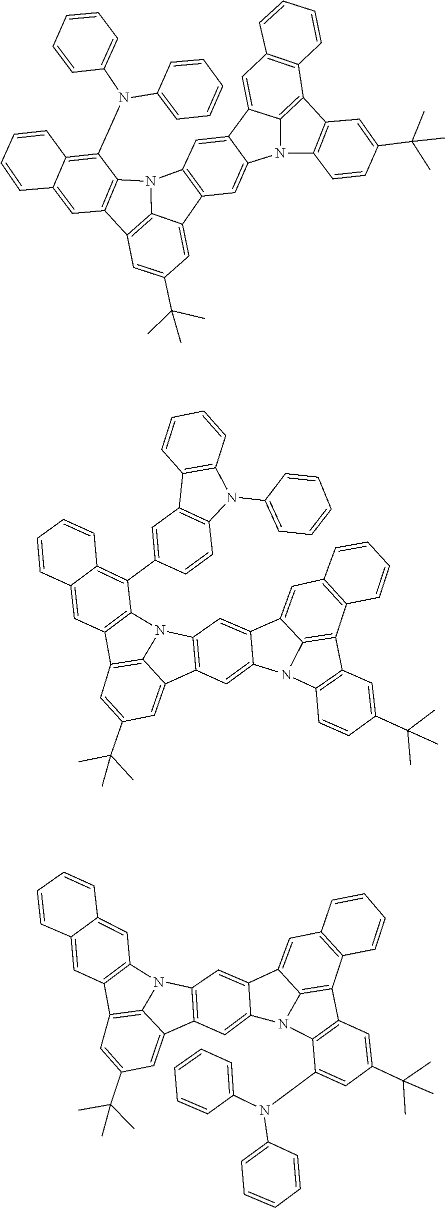 [SMILES: CC(C)(C)c1ccc2c(c1)c1c3ccccc3cc3c4cc5c(cc4n2c31)c1cc(C(C)(C)C)cc2c3cc4ccccc4c(N(c4ccccc4)c4ccccc4)c3n5c12 ; CC(C)(C)c1ccc2c(c1)c1c3ccccc3cc3c4cc5c(cc4n2c31)c1cc(C(C)(C)C)cc2c3cc4ccccc4c(-c4ccc6c(c4)c4ccccc4n6-c4ccccc4)c3n5c12 ; CC(C)(C)c1cc(N(c2ccccc2)c2ccccc2)c2c(c1)c1c3ccccc3cc3c4cc5c(cc4n2c31)c1cc(C(C)(C)C)cc2c3cc4ccccc4cc3n5c21]

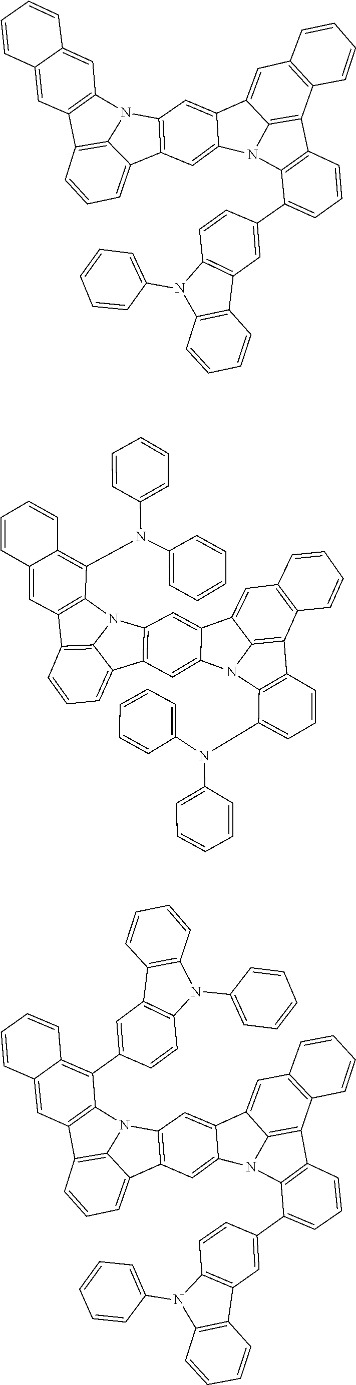 [SMILES: c1ccc(-n2c3ccccc3c3cc(-c4cccc5c6c7ccccc7cc7c8cc9c(cc8n(c45)c76)c4cccc5c6cc7ccccc7cc6n9c54)ccc32)cc1 ; c1ccc(N(c2ccccc2)c2c3ccccc3cc3c4cccc5c6cc7c(cc6n(c23)c54)c2cc3ccccc3c3c4cccc(N(c5ccccc5)c5ccccc5)c4n7c23)cc1 ; c1ccc(-n2c3ccccc3c3cc(-c4c5ccccc5cc5c6cccc7c8cc9c(cc8n(c45)c76)c4cc5ccccc5c5c6cccc(-c7ccc8c(c7)c7ccccc7n8-c7ccccc7)c6n9c45)ccc32)cc1]

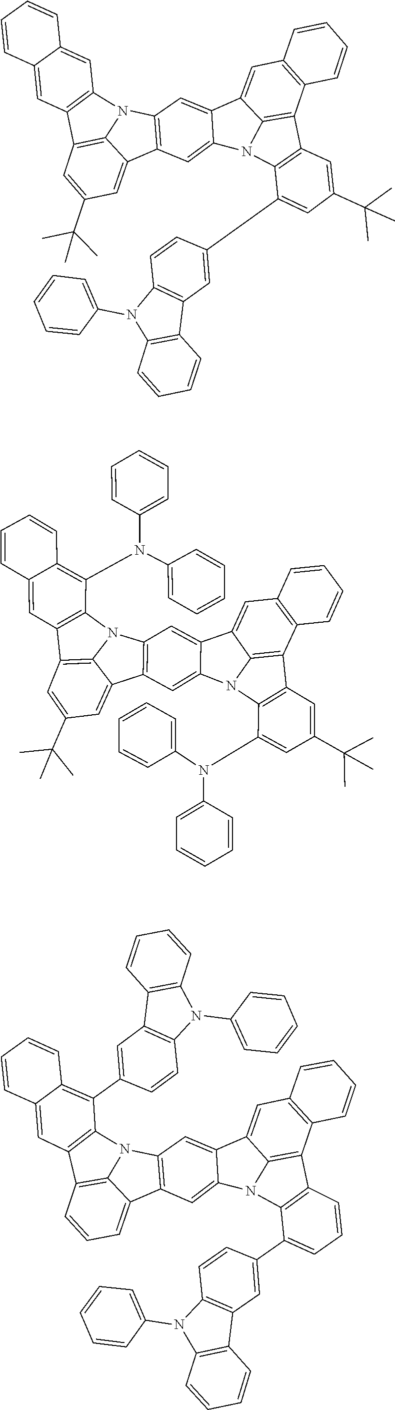 [SMILES: CC(C)(C)c1cc(-c2ccc3c(c2)c2ccccc2n3-c2ccccc2)c2c(c1)c1c3ccccc3cc3c4cc5c(cc4n2c31)c1cc(C(C)(C)C)cc2c3cc4ccccc4cc3n5c21 ; CC(C)(C)c1cc(N(c2ccccc2)c2ccccc2)c2c(c1)c1c3ccccc3cc3c4cc5c(cc4n2c31)c1cc(C(C)(C)C)cc2c3cc4ccccc4c(N(c4ccccc4)c4ccccc4)c3n5c12 ; c1ccc(-n2c3ccccc3c3cc(-c4c5ccccc5cc5c6cccc7c8cc9c(cc8n(c45)c76)c4cc5ccccc5c5c6cccc(-c7ccc8c(c7)c7ccccc7n8-c7ccccc7)c6n9c45)ccc32)cc1]

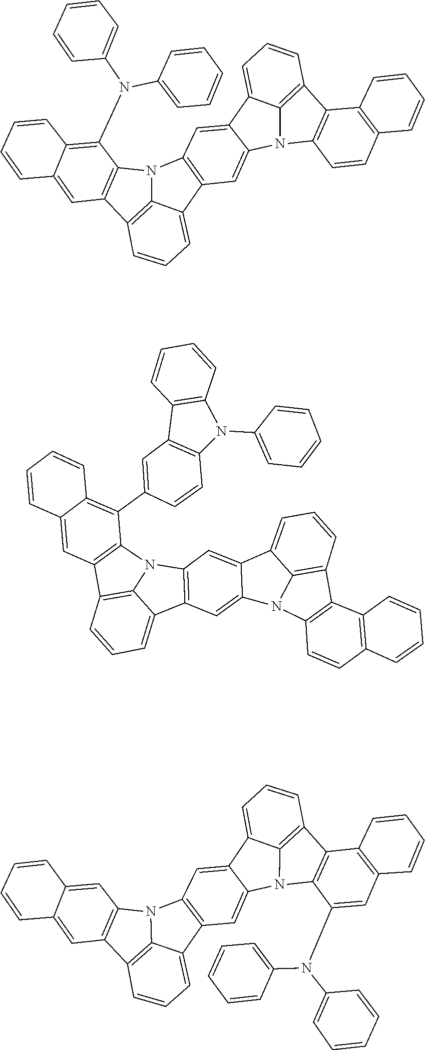 [SMILES: c1ccc(N(c2ccccc2)c2c3ccccc3cc3c4cccc5c6cc7c(cc6n(c23)c54)c2cccc3c4c5ccccc5ccc4n7c23)cc1 ; c1ccc(-n2c3ccccc3c3cc(-c4c5ccccc5cc5c6cccc7c8cc9c(cc8n(c45)c76)c4cccc5c6c7ccccc7ccc6n9c45)ccc32)cc1 ; c1ccc(N(c2ccccc2)c2cc3ccccc3c3c4cccc5c6cc7c(cc6n(c23)c54)c2cccc3c4cc5ccccc5cc4n7c32)cc1]

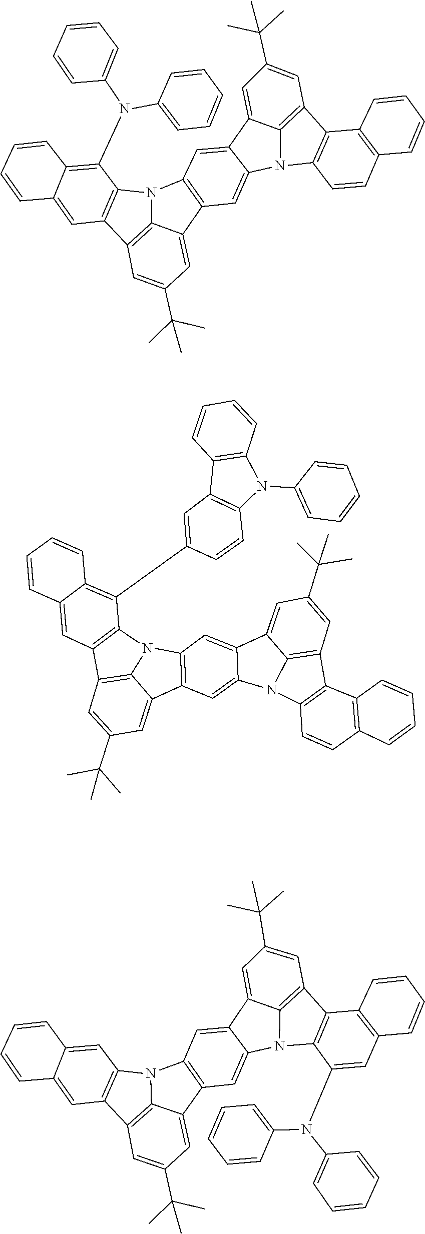 [SMILES: CC(C)(C)c1cc2c3cc4c(cc3n3c5ccc6ccccc6c5c(c1)c23)c1cc(C(C)(C)C)cc2c3cc5ccccc5c(N(c5ccccc5)c5ccccc5)c3n4c12 ; CC(C)(C)c1cc2c3cc4c(cc3n3c5ccc6ccccc6c5c(c1)c23)c1cc(C(C)(C)C)cc2c3cc5ccccc5c(-c5ccc6c(c5)c5ccccc5n6-c5ccccc5)c3n4c12 ; CC(C)(C)c1cc2c3cc4ccccc4cc3n3c4cc5c6cc(C(C)(C)C)cc7c8c9ccccc9cc(N(c9ccccc9)c9ccccc9)c8n(c5cc4c(c1)c23)c67]

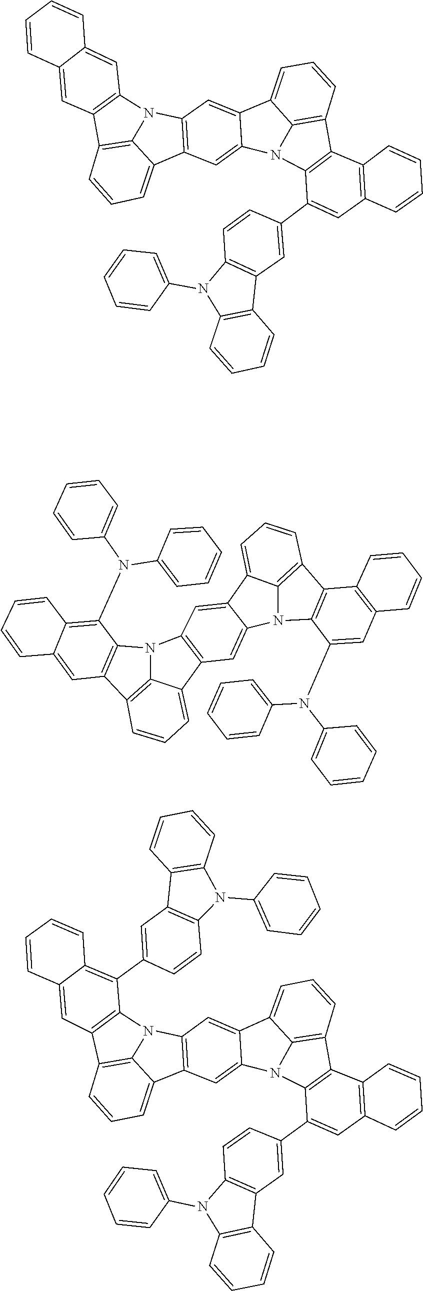 [SMILES: c1ccc(-n2c3ccccc3c3cc(-c4cc5ccccc5c5c6cccc7c8cc9c(cc8n(c45)c76)c4cccc5c6cc7ccccc7cc6n9c54)ccc32)cc1 ; c1ccc(N(c2ccccc2)c2c3ccccc3cc3c4cccc5c6cc7c(cc6n(c23)c54)c2cccc3c4c5ccccc5cc(N(c5ccccc5)c5ccccc5)c4n7c23)cc1 ; c1ccc(-n2c3ccccc3c3cc(-c4c5ccccc5cc5c6cccc7c8cc9c(cc8n(c45)c76)c4cccc5c6c7ccccc7cc(-c7ccc8c(c7)c7ccccc7n8-c7ccccc7)c6n9c45)ccc32)cc1]

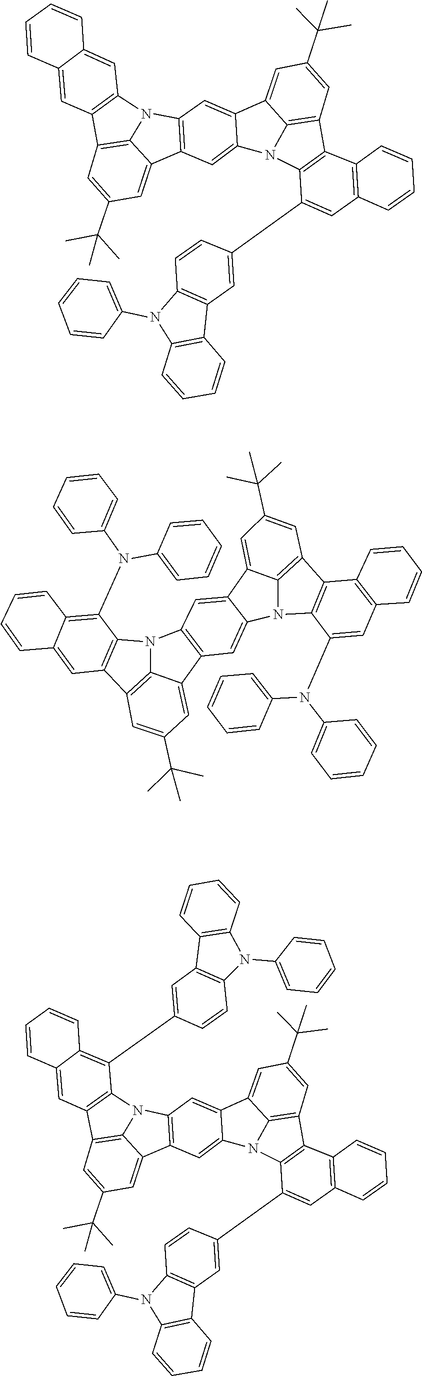 [SMILES: CC(C)(C)c1cc2c3cc4ccccc4cc3n3c4cc5c6cc(C(C)(C)C)cc7c8c9ccccc9cc(-c9ccc%10c(c9)c9ccccc9n%10-c9ccccc9)c8n(c5cc4c(c1)c23)c67 ; CC(C)(C)c1cc2c3cc4c(cc3n3c5c(N(c6ccccc6)c6ccccc6)c6ccccc6cc5c(c1)c23)c1cc(C(C)(C)C)cc2c3c5ccccc5cc(N(c5ccccc5)c5ccccc5)c3n4c12 ; CC(C)(C)c1cc2c3cc4c(cc3n3c5c(-c6ccc7c(c6)c6ccccc6n7-c6ccccc6)c6ccccc6cc5c(c1)c23)c1cc(C(C)(C)C)cc2c3c5ccccc5cc(-c5ccc6c(c5)c5ccccc5n6-c5ccccc5)c3n4c12]

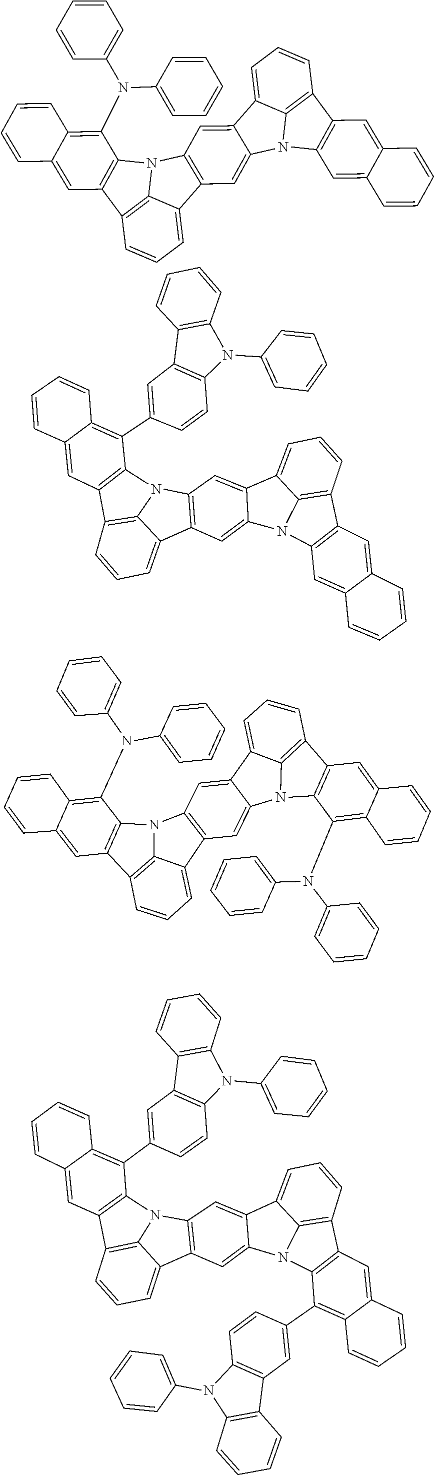

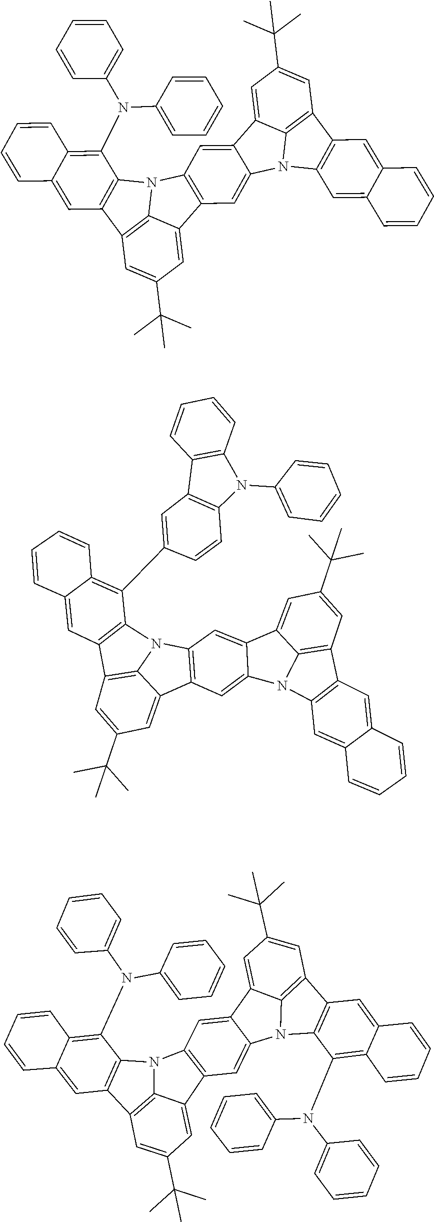 [SMILES: CC(C)(C)c1cc2c3cc4ccccc4cc3n3c4cc5c6cc(C(C)(C)C)cc7c8cc9ccccc9c(N(c9ccccc9)c9ccccc9)c8n(c5cc4c(c1)c23)c67 ; CC(C)(C)c1cc2c3cc4ccccc4cc3n3c4cc5c6cc(C(C)(C)C)cc7c8cc9ccccc9c(-c9ccc%10c(c9)c9ccccc9n%10-c9ccccc9)c8n(c5cc4c(c1)c23)c67 ; CC(C)(C)c1cc2c3cc4c(cc3n3c5c(N(c6ccccc6)c6ccccc6)c6ccccc6cc5c(c1)c23)c1cc(C(C)(C)C)cc2c3cc5ccccc5c(N(c5ccccc5)c5ccccc5)c3n4c12]

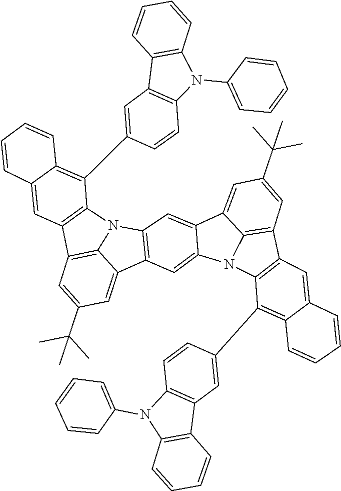 [SMILES: CC(C)(C)c1cc2c3cc4c(cc3n3c5c(-c6ccc7c(c6)c6ccccc6n7-c6ccccc6)c6ccccc6cc5c(c1)c23)c1cc(C(C)(C)C)cc2c3cc5ccccc5c(-c5ccc6c(c5)c5ccccc5n6-c5ccccc5)c3n4c12]

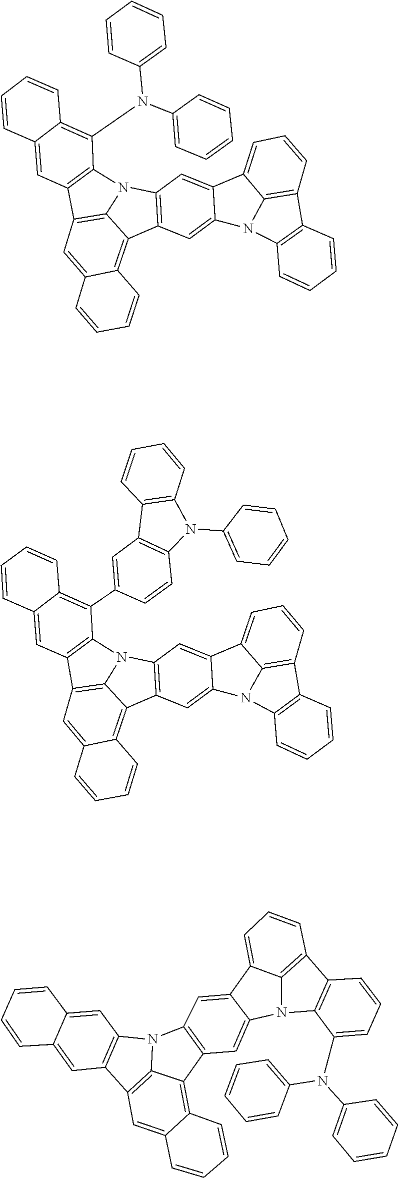 [SMILES: c1ccc(N(c2ccccc2)c2c3ccccc3cc3c4cc5ccccc5c5c6cc7c(cc6n(c23)c45)c2cccc3c4ccccc4n7c32)cc1 ; c1ccc(-n2c3ccccc3c3cc(-c4c5ccccc5cc5c6cc7ccccc7c7c8cc9c(cc8n(c45)c67)c4cccc5c6ccccc6n9c54)ccc32)cc1 ; c1ccc(N(c2ccccc2)c2cccc3c4cccc5c6cc7c(cc6n(c23)c54)c2c3ccccc3cc3c4cc5ccccc5cc4n7c32)cc1]

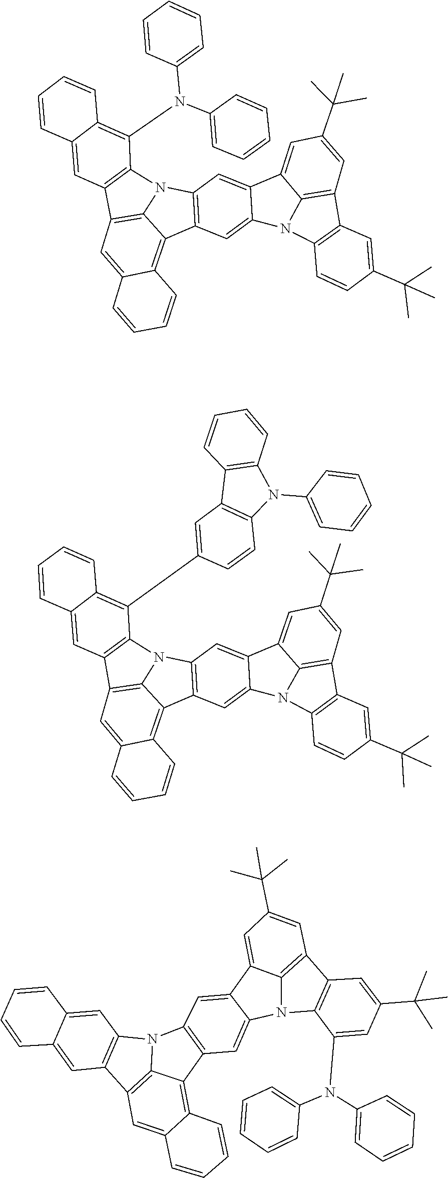 [SMILES: CC(C)(C)c1ccc2c(c1)c1cc(C(C)(C)C)cc3c4cc5c(cc4n2c13)c1c2ccccc2cc2c3cc4ccccc4c(N(c4ccccc4)c4ccccc4)c3n5c21 ; CC(C)(C)c1ccc2c(c1)c1cc(C(C)(C)C)cc3c4cc5c(cc4n2c13)c1c2ccccc2cc2c3cc4ccccc4c(-c4ccc6c(c4)c4ccccc4n6-c4ccccc4)c3n5c21 ; CC(C)(C)c1cc(N(c2ccccc2)c2ccccc2)c2c(c1)c1cc(C(C)(C)C)cc3c4cc5c(cc4n2c31)c1c2ccccc2cc2c3cc4ccccc4cc3n5c21]

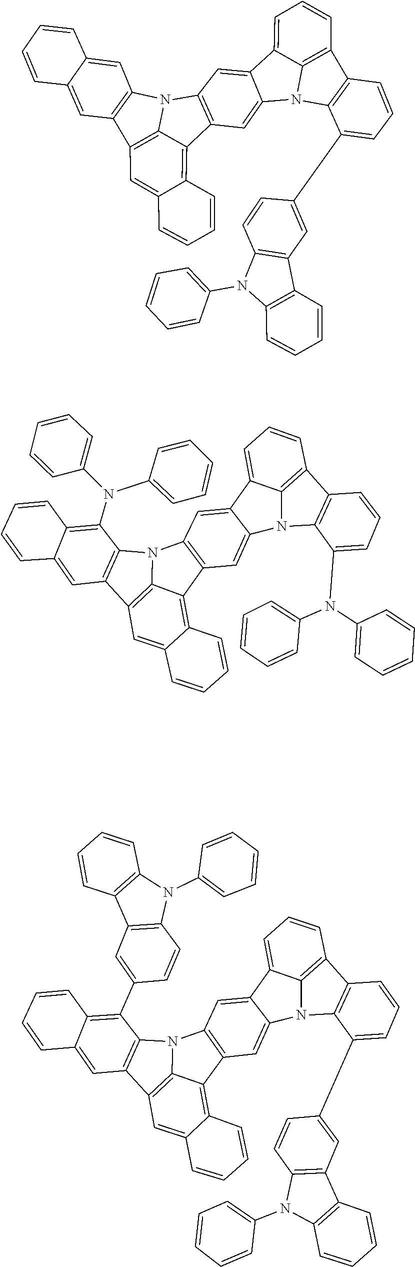 [SMILES: c1ccc(-n2c3ccccc3c3cc(-c4cccc5c6cccc7c8cc9c(cc8n(c45)c76)c4c5ccccc5cc5c6cc7ccccc7cc6n9c54)ccc32)cc1 ; c1ccc(N(c2ccccc2)c2cccc3c4cccc5c6cc7c(cc6n(c23)c54)c2c3ccccc3cc3c4cc5ccccc5c(N(c5ccccc5)c5ccccc5)c4n7c32)cc1 ; c1ccc(-n2c3ccccc3c3cc(-c4cccc5c6cccc7c8cc9c(cc8n(c45)c76)c4c5ccccc5cc5c6cc7ccccc7c(-c7ccc8c(c7)c7ccccc7n8-c7ccccc7)c6n9c54)ccc32)cc1]

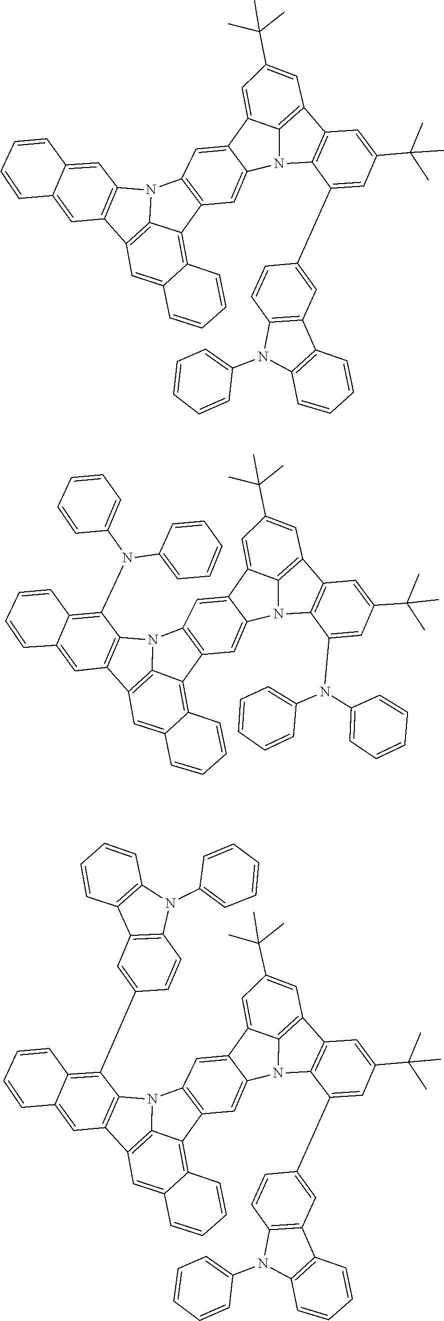 [SMILES: CC(C)(C)c1cc(-c2ccc3c(c2)c2ccccc2n3-c2ccccc2)c2c(c1)c1cc(C(C)(C)C)cc3c4cc5c(cc4n2c31)c1c2ccccc2cc2c3cc4ccccc4cc3n5c21 ; CC(C)(C)c1cc(N(c2ccccc2)c2ccccc2)c2c(c1)c1cc(C(C)(C)C)cc3c4cc5c(cc4n2c31)c1c2ccccc2cc2c3cc4ccccc4c(N(c4ccccc4)c4ccccc4)c3n5c21 ; CC(C)(C)c1cc(-c2ccc3c(c2)c2ccccc2n3-c2ccccc2)c2c(c1)c1cc(C(C)(C)C)cc3c4cc5c(cc4n2c31)c1c2ccccc2cc2c3cc4ccccc4c(-c4ccc6c(c4)c4ccccc4n6-c4ccccc4)c3n5c21]

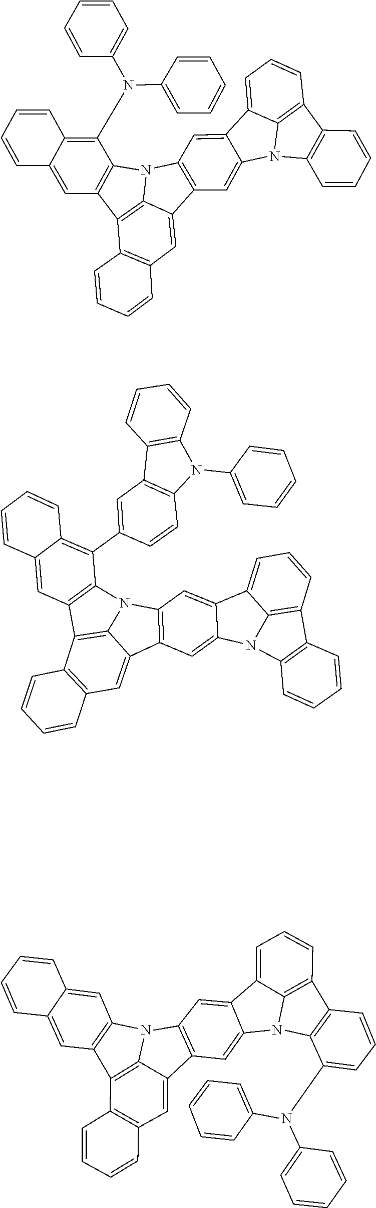 [SMILES: c1ccc(N(c2ccccc2)c2c3ccccc3cc3c4c5ccccc5cc5c6cc7c(cc6n(c23)c54)c2cccc3c4ccccc4n7c32)cc1 ; c1ccc(-n2c3ccccc3c3cc(-c4c5ccccc5cc5c6c7ccccc7cc7c8cc9c(cc8n(c45)c76)c4cccc5c6ccccc6n9c54)ccc32)cc1 ; c1ccc(N(c2ccccc2)c2cccc3c4cccc5c6cc7c(cc6n(c23)c54)c2cc3ccccc3c3c4cc5ccccc5cc4n7c23)cc1]

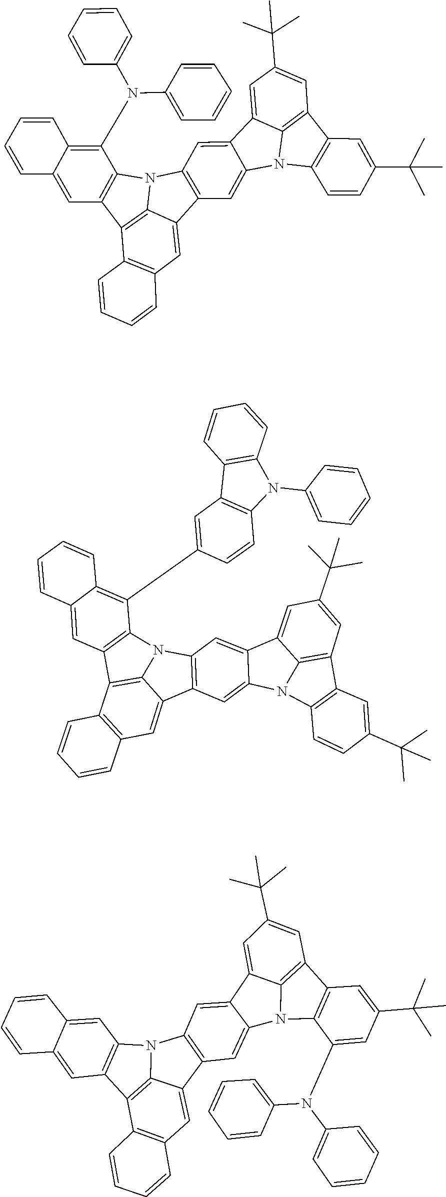 [SMILES: CC(C)(C)c1ccc2c(c1)c1cc(C(C)(C)C)cc3c4cc5c(cc4n2c13)c1cc2ccccc2c2c3cc4ccccc4c(N(c4ccccc4)c4ccccc4)c3n5c12 ; CC(C)(C)c1ccc2c(c1)c1cc(C(C)(C)C)cc3c4cc5c(cc4n2c13)c1cc2ccccc2c2c3cc4ccccc4c(-c4ccc6c(c4)c4ccccc4n6-c4ccccc4)c3n5c12 ; CC(C)(C)c1cc(N(c2ccccc2)c2ccccc2)c2c(c1)c1cc(C(C)(C)C)cc3c4cc5c(cc4n2c31)c1cc2ccccc2c2c3cc4ccccc4cc3n5c12]

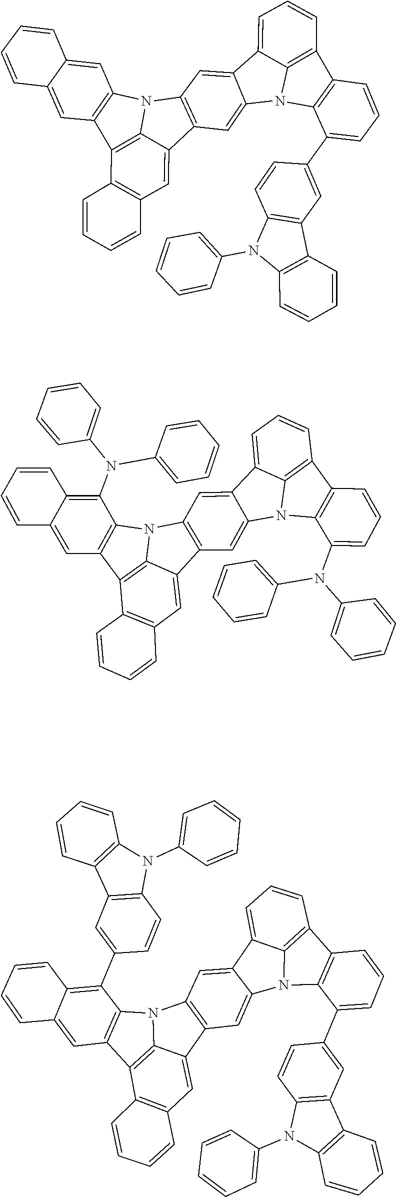 [SMILES: c1ccc(-n2c3ccccc3c3cc(-c4cccc5c6cccc7c8cc9c(cc8n(c45)c76)c4cc5ccccc5c5c6cc7ccccc7cc6n9c45)ccc32)cc1 ; c1ccc(N(c2ccccc2)c2cccc3c4cccc5c6cc7c(cc6n(c23)c54)c2cc3ccccc3c3c4cc5ccccc5c(N(c5ccccc5)c5ccccc5)c4n7c23)cc1 ; c1ccc(-n2c3ccccc3c3cc(-c4cccc5c6cccc7c8cc9c(cc8n(c45)c76)c4cc5ccccc5c5c6cc7ccccc7c(-c7ccc8c(c7)c7ccccc7n8-c7ccccc7)c6n9c45)ccc32)cc1]

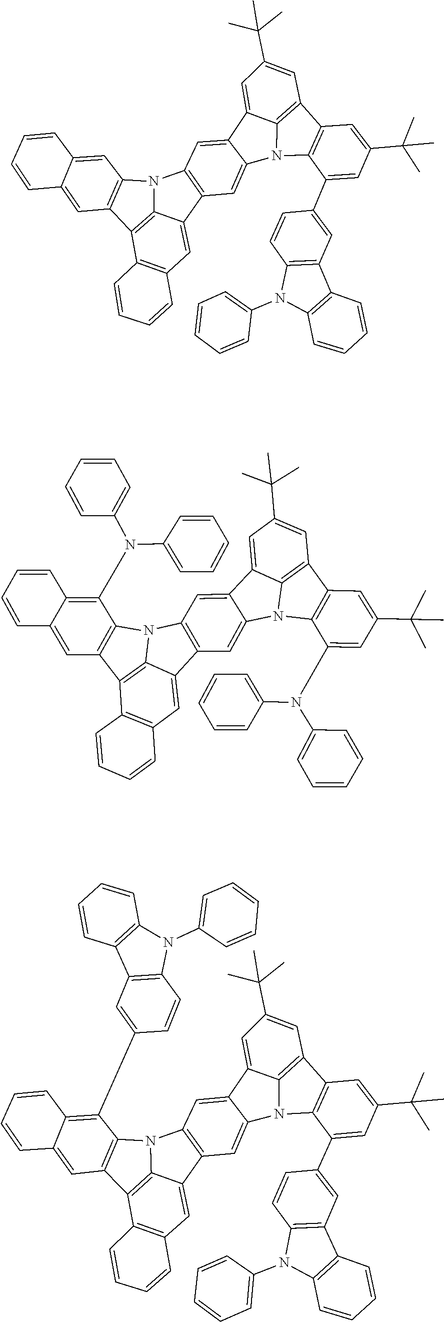 [SMILES: CC(C)(C)c1cc(-c2ccc3c(c2)c2ccccc2n3-c2ccccc2)c2c(c1)c1cc(C(C)(C)C)cc3c4cc5c(cc4n2c31)c1cc2ccccc2c2c3cc4ccccc4cc3n5c12 ; CC(C)(C)c1cc(N(c2ccccc2)c2ccccc2)c2c(c1)c1cc(C(C)(C)C)cc3c4cc5c(cc4n2c31)c1cc2ccccc2c2c3cc4ccccc4c(N(c4ccccc4)c4ccccc4)c3n5c12 ; CC(C)(C)c1cc(-c2ccc3c(c2)c2ccccc2n3-c2ccccc2)c2c(c1)c1cc(C(C)(C)C)cc3c4cc5c(cc4n2c31)c1cc2ccccc2c2c3cc4ccccc4c(-c4ccc6c(c4)c4ccccc4n6-c4ccccc4)c3n5c12]

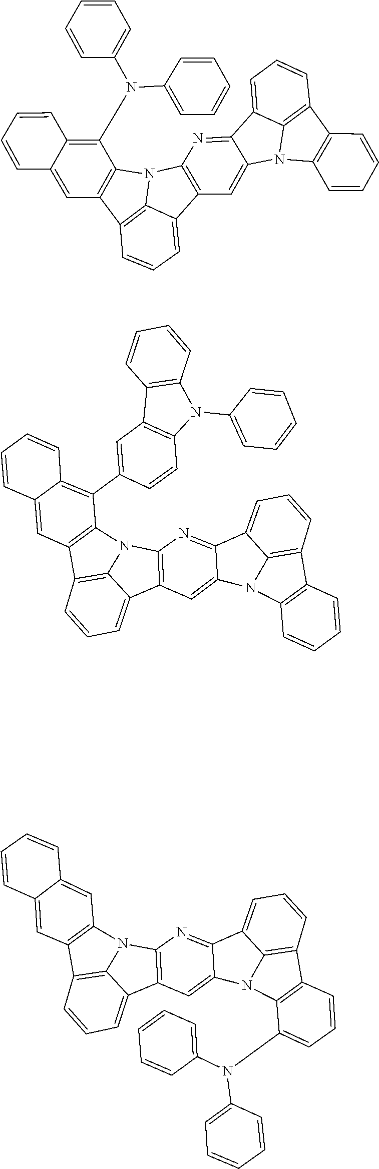 [SMILES: c1ccc(N(c2ccccc2)c2c3ccccc3cc3c4cccc5c6cc7c(nc6n(c23)c54)c2cccc3c4ccccc4n7c32)cc1 ; c1ccc(-n2c3ccccc3c3cc(-c4c5ccccc5cc5c6cccc7c8cc9c(nc8n(c45)c76)c4cccc5c6ccccc6n9c54)ccc32)cc1 ; c1ccc(N(c2ccccc2)c2cccc3c4cccc5c6nc7c(cc6n(c23)c45)c2cccc3c4cc5ccccc5cc4n7c32)cc1]

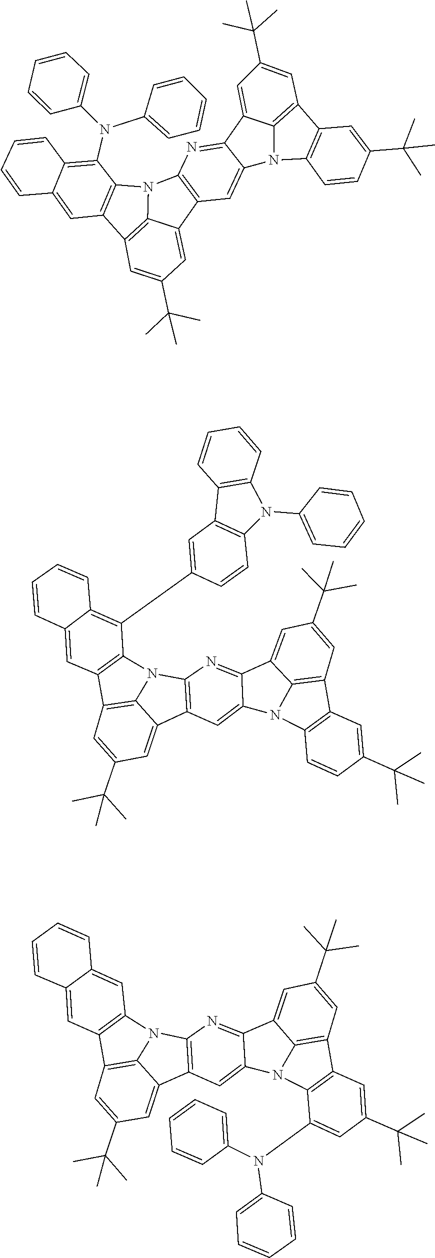 [SMILES: CC(C)(C)c1ccc2c(c1)c1cc(C(C)(C)C)cc3c4nc5c(cc4n2c13)c1cc(C(C)(C)C)cc2c3cc4ccccc4c(N(c4ccccc4)c4ccccc4)c3n5c12 ; CC(C)(C)c1ccc2c(c1)c1cc(C(C)(C)C)cc3c4nc5c(cc4n2c13)c1cc(C(C)(C)C)cc2c3cc4ccccc4c(-c4ccc6c(c4)c4ccccc4n6-c4ccccc4)c3n5c12 ; CC(C)(C)c1cc(N(c2ccccc2)c2ccccc2)c2c(c1)c1cc(C(C)(C)C)cc3c4nc5c(cc4n2c13)c1cc(C(C)(C)C)cc2c3cc4ccccc4cc3n5c21]

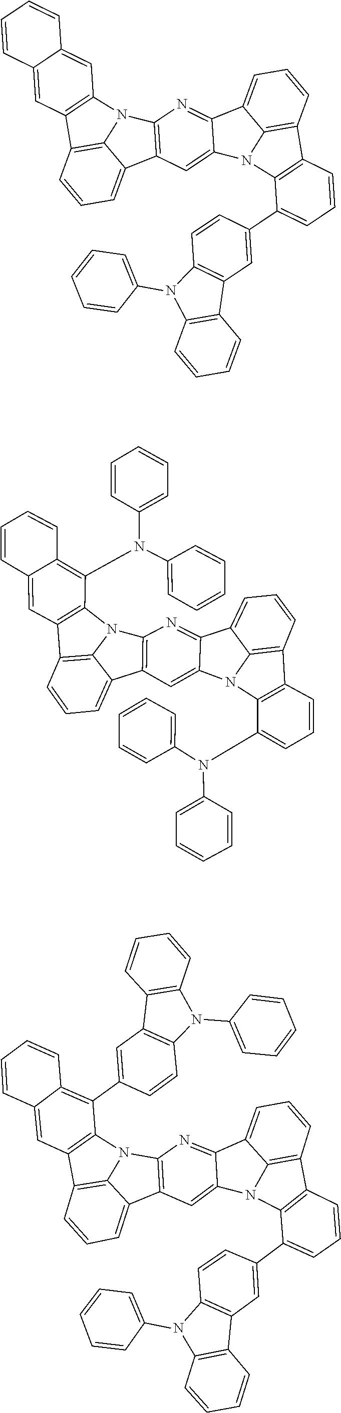 [SMILES: c1ccc(-n2c3ccccc3c3cc(-c4cccc5c6cccc7c8nc9c(cc8n(c45)c67)c4cccc5c6cc7ccccc7cc6n9c54)ccc32)cc1 ; c1ccc(N(c2ccccc2)c2cccc3c4cccc5c6nc7c(cc6n(c23)c45)c2cccc3c4cc5ccccc5c(N(c5ccccc5)c5ccccc5)c4n7c23)cc1 ; c1ccc(-n2c3ccccc3c3cc(-c4cccc5c6cccc7c8nc9c(cc8n(c45)c67)c4cccc5c6cc7ccccc7c(-c7ccc8c(c7)c7ccccc7n8-c7ccccc7)c6n9c45)ccc32)cc1]

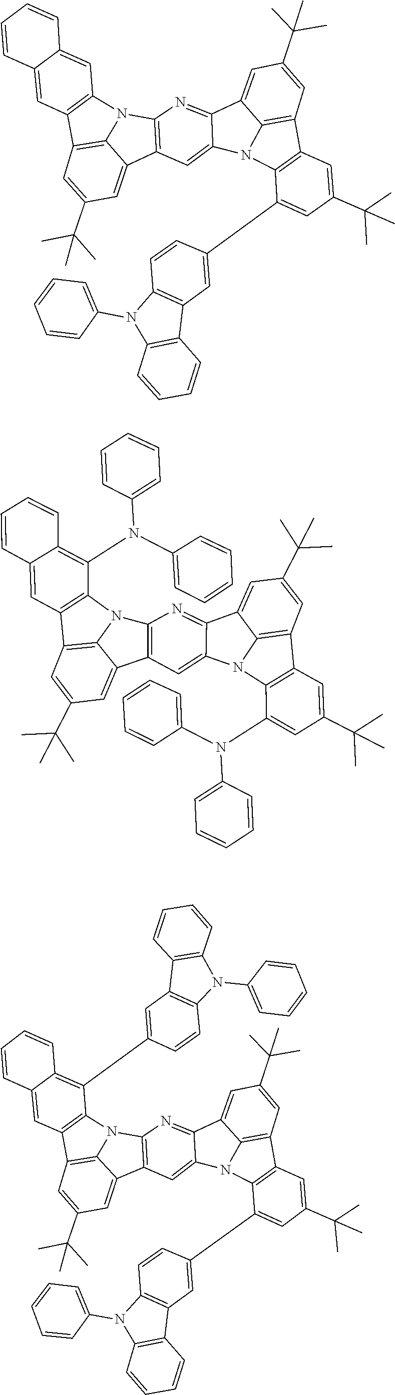 [SMILES: CC(C)(C)c1cc(-c2ccc3c(c2)c2ccccc2n3-c2ccccc2)c2c(c1)c1cc(C(C)(C)C)cc3c4nc5c(cc4n2c13)c1cc(C(C)(C)C)cc2c3cc4ccccc4cc3n5c21 ; CC(C)(C)c1cc(N(c2ccccc2)c2ccccc2)c2c(c1)c1cc(C(C)(C)C)cc3c4nc5c(cc4n2c13)c1cc(C(C)(C)C)cc2c3cc4ccccc4c(N(c4ccccc4)c4ccccc4)c3n5c12 ; CC(C)(C)c1cc(-c2ccc3c(c2)c2ccccc2n3-c2ccccc2)c2c(c1)c1cc(C(C)(C)C)cc3c4nc5c(cc4n2c13)c1cc(C(C)(C)C)cc2c3cc4ccccc4c(-c4ccc6c(c4)c4ccccc4n6-c4ccccc4)c3n5c12]

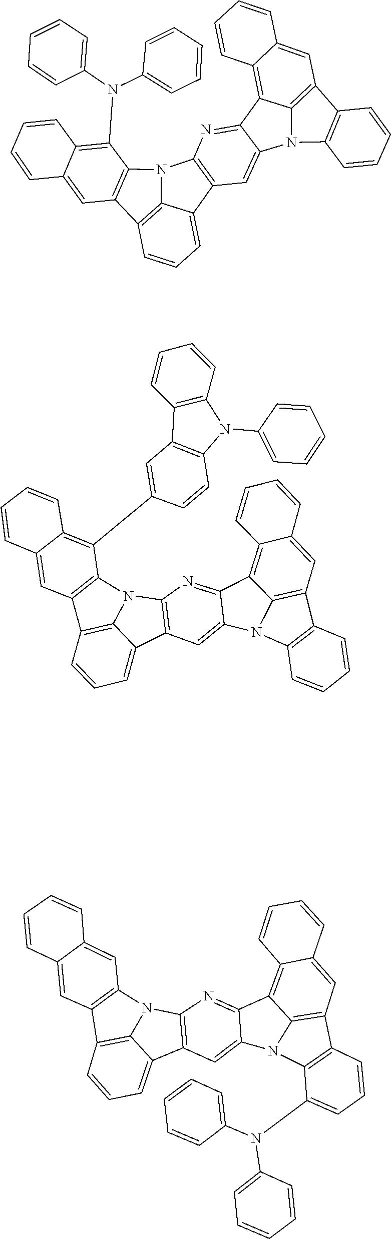 [SMILES: c1ccc(N(c2ccccc2)c2c3ccccc3cc3c4cccc5c6cc7c(nc6n(c23)c54)c2c3ccccc3cc3c4ccccc4n7c32)cc1 ; c1ccc(-n2c3ccccc3c3cc(-c4c5ccccc5cc5c6cccc7c8cc9c(nc8n(c45)c76)c4c5ccccc5cc5c6ccccc6n9c54)ccc32)cc1 ; c1ccc(N(c2ccccc2)c2cccc3c4cc5ccccc5c5c6nc7c(cc6n(c23)c45)c2cccc3c4cc5ccccc5cc4n7c32)cc1]

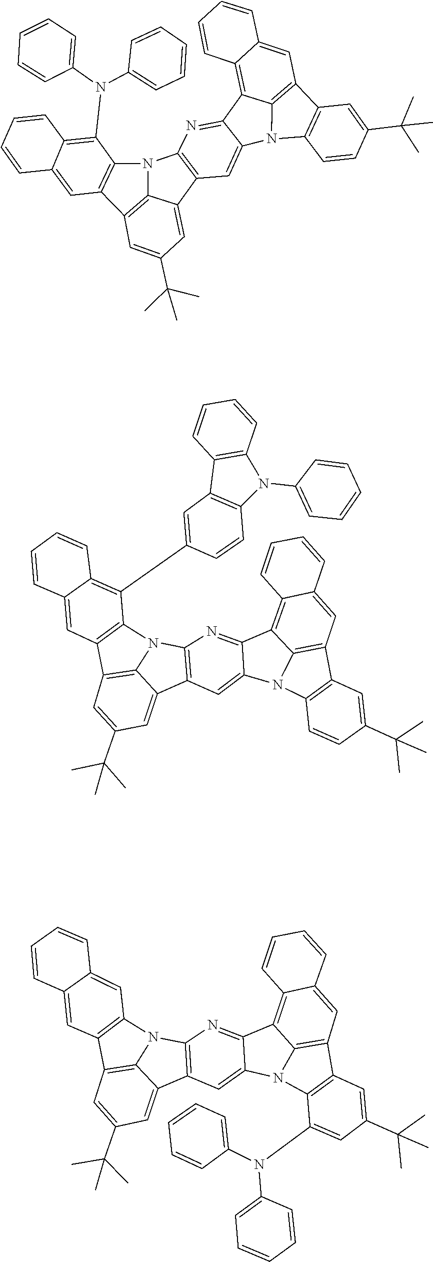 [SMILES: CC(C)(C)c1ccc2c(c1)c1cc3ccccc3c3c4nc5c(cc4n2c13)c1cc(C(C)(C)C)cc2c3cc4ccccc4c(N(c4ccccc4)c4ccccc4)c3n5c12 ; CC(C)(C)c1ccc2c(c1)c1cc3ccccc3c3c4nc5c(cc4n2c13)c1cc(C(C)(C)C)cc2c3cc4ccccc4c(-c4ccc6c(c4)c4ccccc4n6-c4ccccc4)c3n5c12 ; CC(C)(C)c1cc(N(c2ccccc2)c2ccccc2)c2c(c1)c1cc3ccccc3c3c4nc5c(cc4n2c13)c1cc(C(C)(C)C)cc2c3cc4ccccc4cc3n5c21]

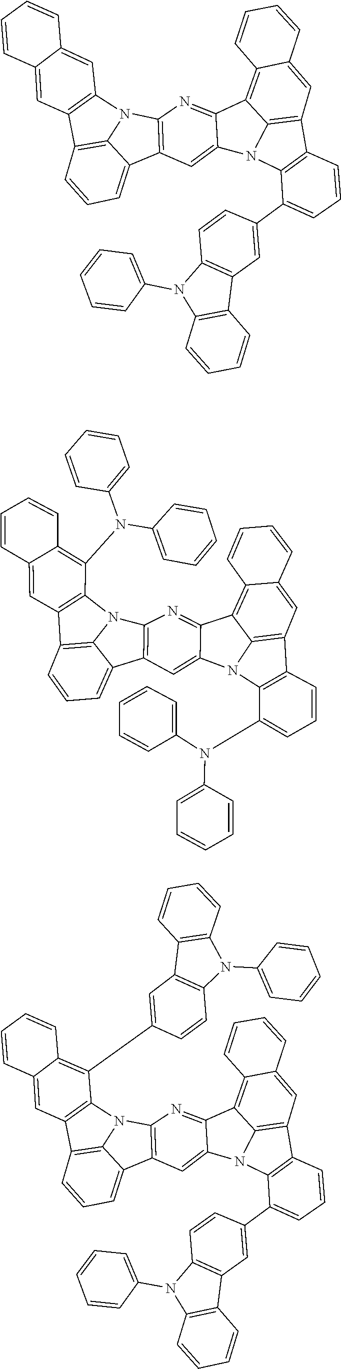

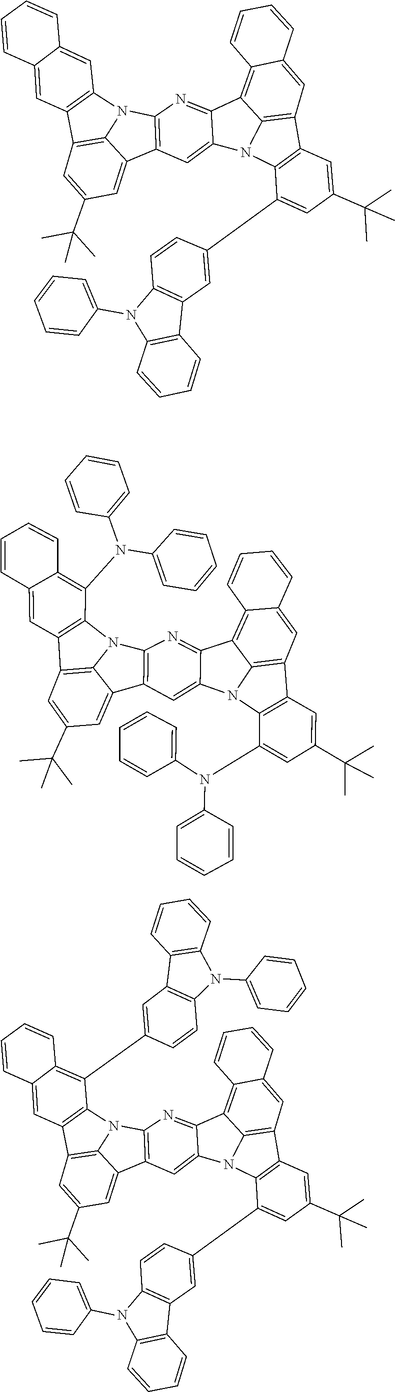

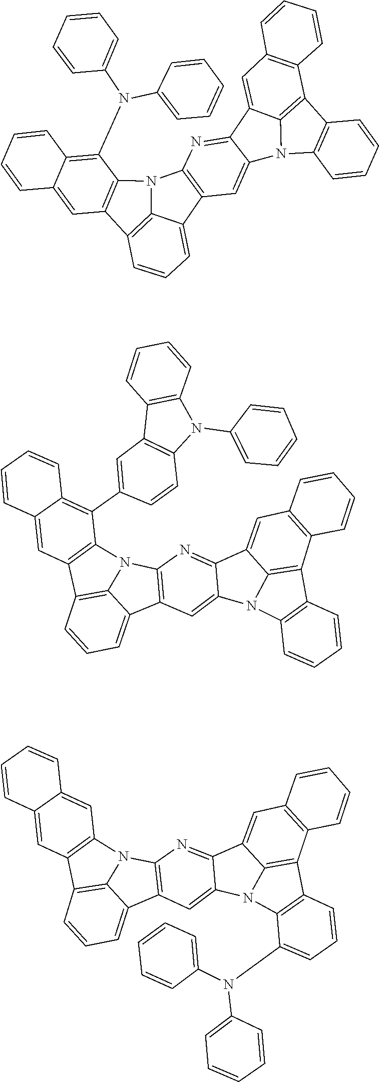 [SMILES: c1ccc(N(c2ccccc2)c2c3ccccc3cc3c4cccc5c6cc7c(nc6n(c23)c54)c2cc3ccccc3c3c4ccccc4n7c23)cc1 ; c1ccc(-n2c3ccccc3c3cc(-c4c5ccccc5cc5c6cccc7c8cc9c(nc8n(c45)c76)c4cc5ccccc5c5c6ccccc6n9c45)ccc32)cc1 ; c1ccc(N(c2ccccc2)c2cccc3c4c5ccccc5cc5c6nc7c(cc6n(c23)c54)c2cccc3c4cc5ccccc5cc4n7c32)cc1]

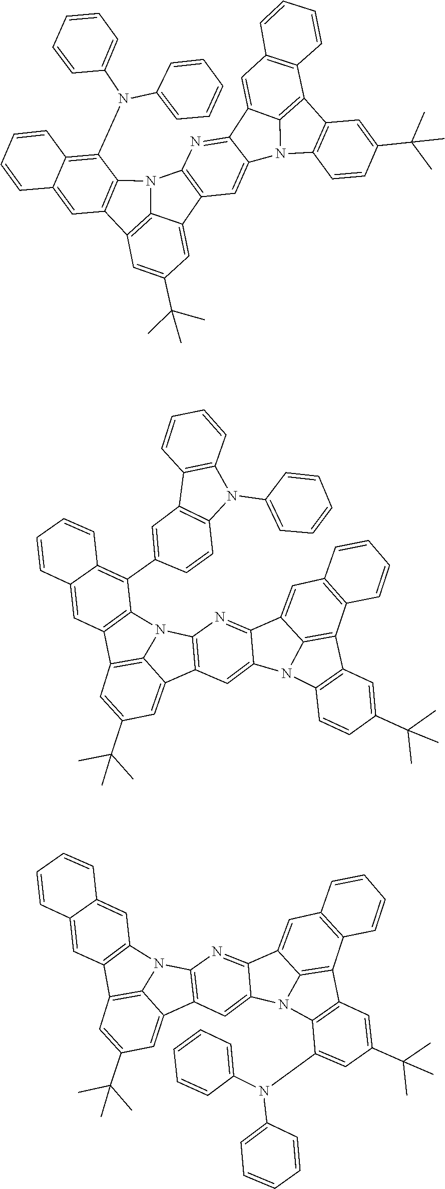 [SMILES: CC(C)(C)c1ccc2c(c1)c1c3ccccc3cc3c4nc5c(cc4n2c31)c1cc(C(C)(C)C)cc2c3cc4ccccc4c(N(c4ccccc4)c4ccccc4)c3n5c12 ; CC(C)(C)c1ccc2c(c1)c1c3ccccc3cc3c4nc5c(cc4n2c31)c1cc(C(C)(C)C)cc2c3cc4ccccc4c(-c4ccc6c(c4)c4ccccc4n6-c4ccccc4)c3n5c12 ; CC(C)(C)c1cc(N(c2ccccc2)c2ccccc2)c2c(c1)c1c3ccccc3cc3c4nc5c(cc4n2c31)c1cc(C(C)(C)C)cc2c3cc4ccccc4cc3n5c21]

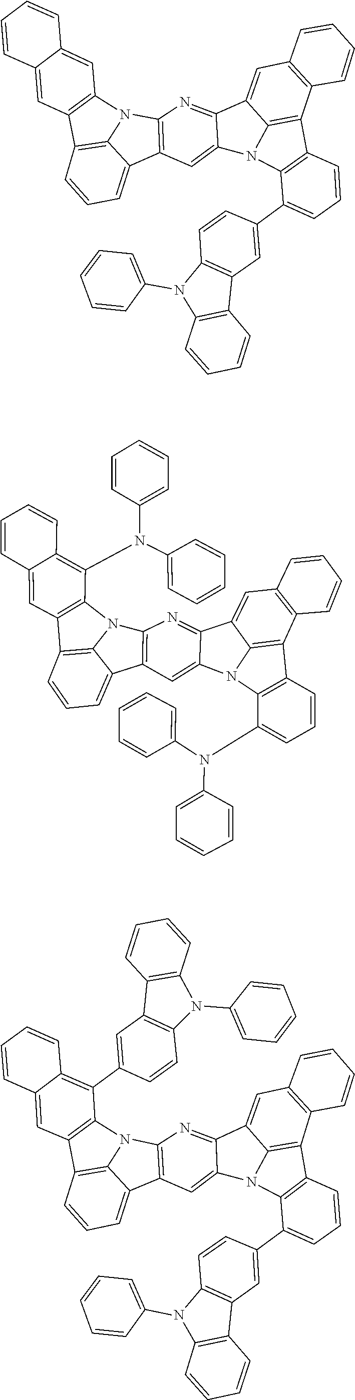 [SMILES: c1ccc(-n2c3ccccc3c3cc(-c4cccc5c6c7ccccc7cc7c8nc9c(cc8n(c45)c76)c4cccc5c6cc7ccccc7cc6n9c54)ccc32)cc1 ; c1ccc(N(c2ccccc2)c2cccc3c4c5ccccc5cc5c6nc7c(cc6n(c23)c54)c2cccc3c4cc5ccccc5c(N(c5ccccc5)c5ccccc5)c4n7c23)cc1 ; c1ccc(-n2c3ccccc3c3cc(-c4cccc5c6c7ccccc7cc7c8nc9c(cc8n(c45)c76)c4cccc5c6cc7ccccc7c(-c7ccc8c(c7)c7ccccc7n8-c7ccccc7)c6n9c45)ccc32)cc1]

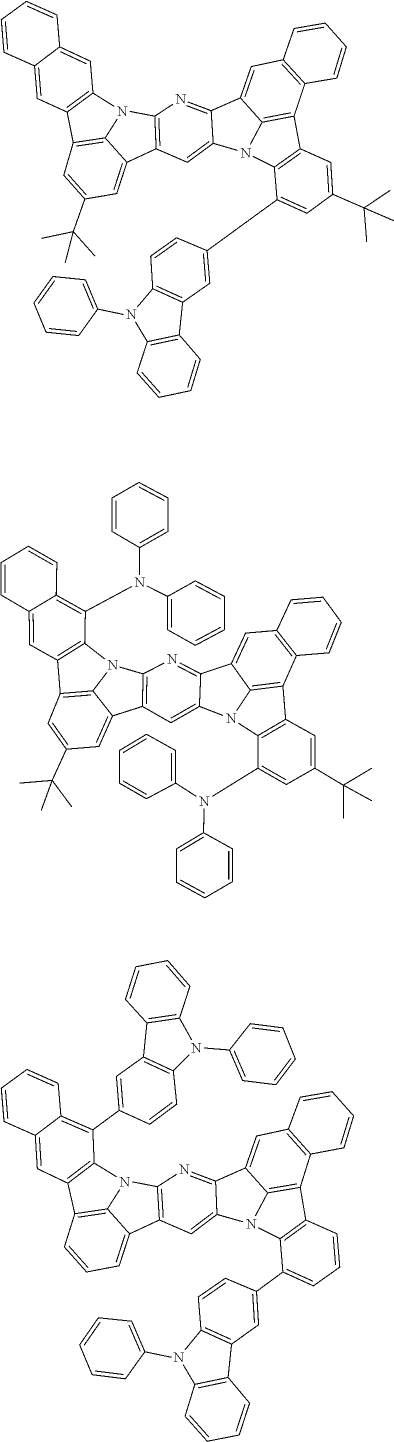 [SMILES: CC(C)(C)c1cc(-c2ccc3c(c2)c2ccccc2n3-c2ccccc2)c2c(c1)c1c3ccccc3cc3c4nc5c(cc4n2c31)c1cc(C(C)(C)C)cc2c3cc4ccccc4cc3n5c21 ; CC(C)(C)c1cc(N(c2ccccc2)c2ccccc2)c2c(c1)c1c3ccccc3cc3c4nc5c(cc4n2c31)c1cc(C(C)(C)C)cc2c3cc4ccccc4c(N(c4ccccc4)c4ccccc4)c3n5c12 ; c1ccc(-n2c3ccccc3c3cc(-c4cccc5c6c7ccccc7cc7c8nc9c(cc8n(c45)c76)c4cccc5c6cc7ccccc7c(-c7ccc8c(c7)c7ccccc7n8-c7ccccc7)c6n9c45)ccc32)cc1]

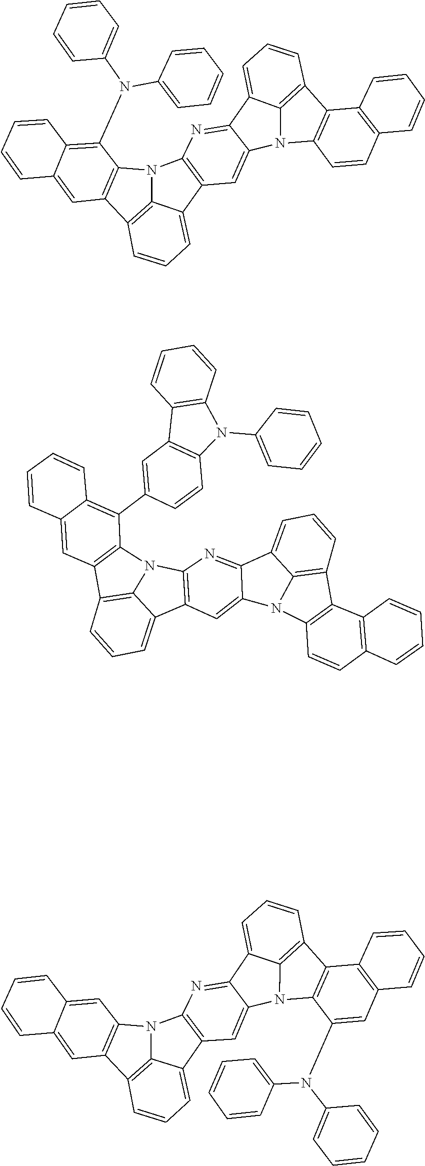 [SMILES: c1ccc(N(c2ccccc2)c2c3ccccc3cc3c4cccc5c6cc7c(nc6n(c23)c54)c2cccc3c4c5ccccc5ccc4n7c23)cc1 ; c1ccc(-n2c3ccccc3c3cc(-c4c5ccccc5cc5c6cccc7c8cc9c(nc8n(c45)c76)c4cccc5c6c7ccccc7ccc6n9c45)ccc32)cc1 ; c1ccc(N(c2ccccc2)c2cc3ccccc3c3c4cccc5c6nc7c(cc6n(c23)c54)c2cccc3c4cc5ccccc5cc4n7c32)cc1]

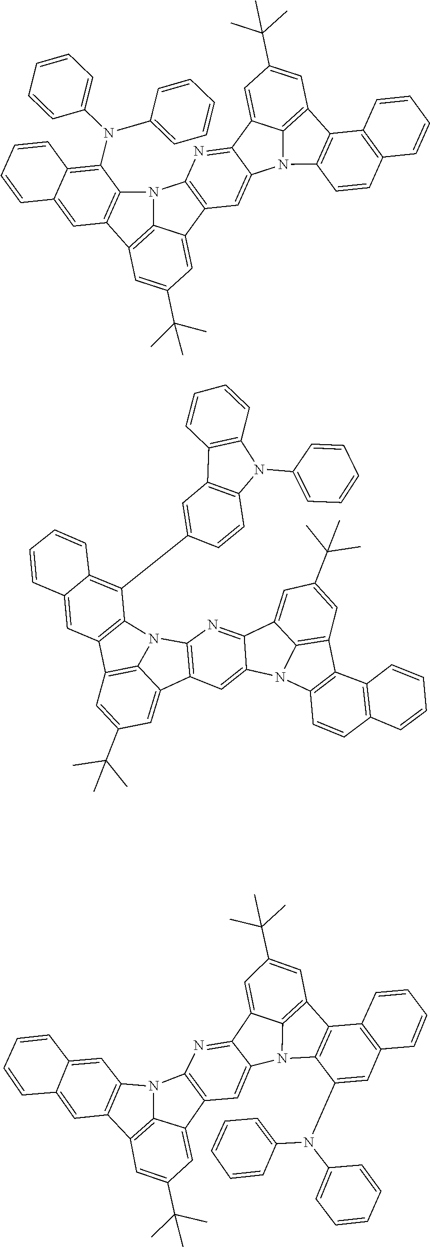

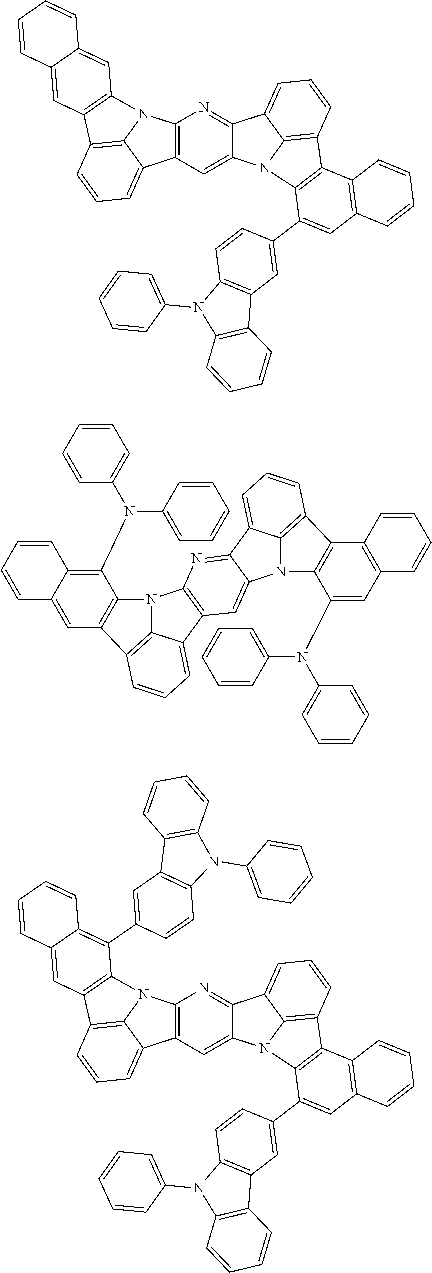 [SMILES: c1ccc(-n2c3ccccc3c3cc(-c4cc5ccccc5c5c6cccc7c8nc9c(cc8n(c45)c76)c4cccc5c6cc7ccccc7cc6n9c54)ccc32)cc1 ; c1ccc(N(c2ccccc2)c2cc3ccccc3c3c4cccc5c6nc7c(cc6n(c23)c54)c2cccc3c4cc5ccccc5c(N(c5ccccc5)c5ccccc5)c4n7c23)cc1 ; c1ccc(-n2c3ccccc3c3cc(-c4cc5ccccc5c5c6cccc7c8nc9c(cc8n(c45)c76)c4cccc5c6cc7ccccc7c(-c7ccc8c(c7)c7ccccc7n8-c7ccccc7)c6n9c45)ccc32)cc1]

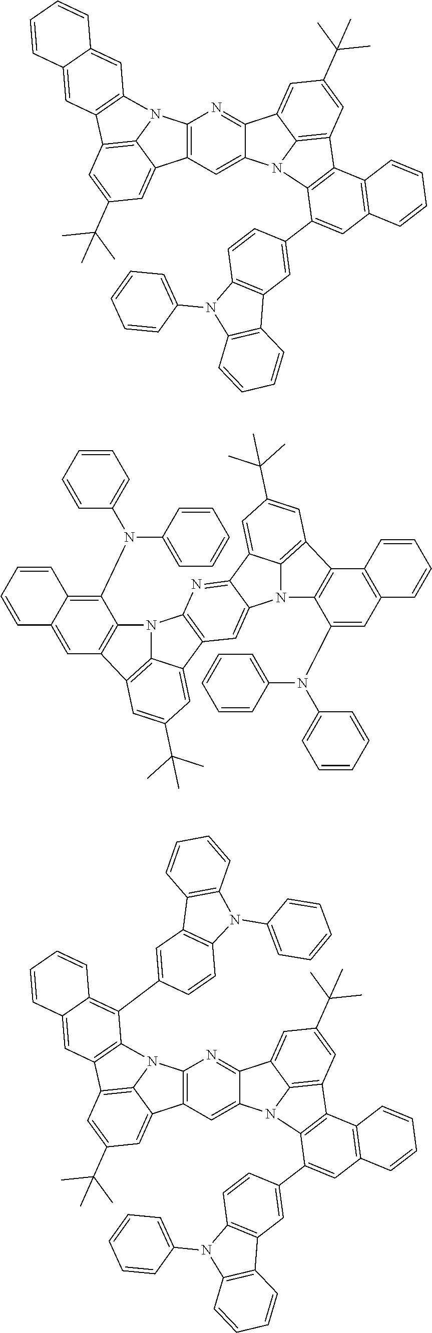

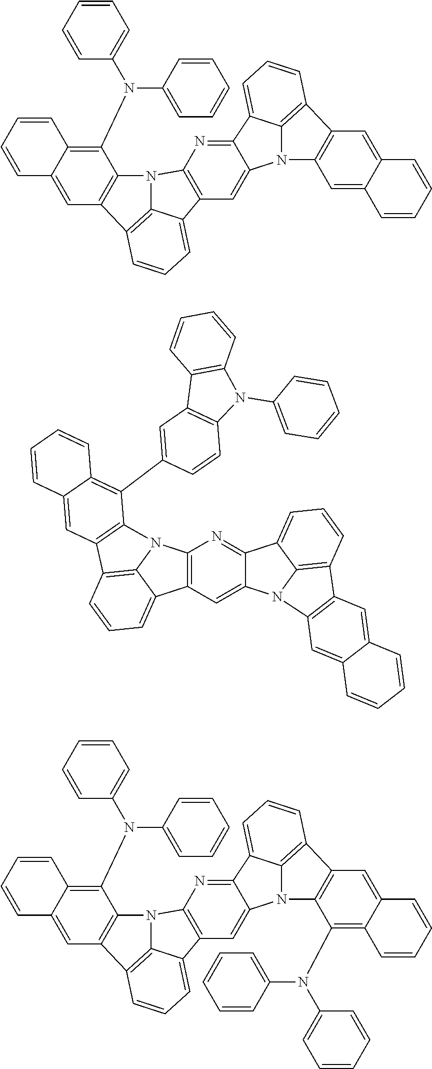

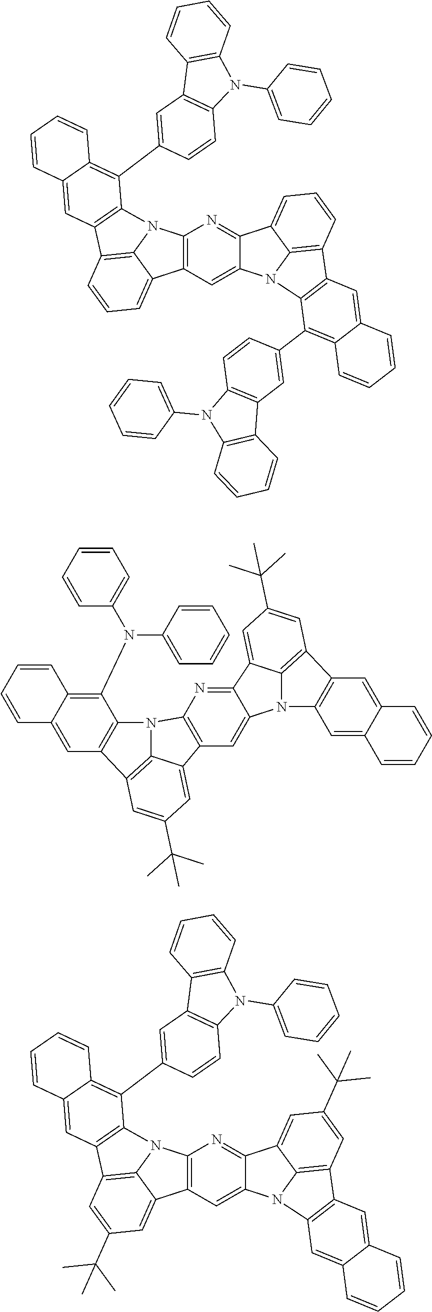

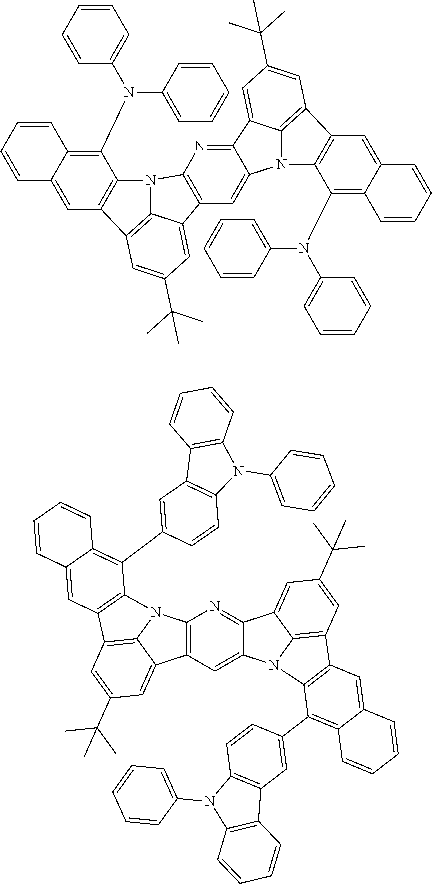 [SMILES: CC(C)(C)c1cc2c3cc4ccccc4c(N(c4ccccc4)c4ccccc4)c3n3c4cc5c6cc(C(C)(C)C)cc7c8cc9ccccc9c(N(c9ccccc9)c9ccccc9)c8n(c5nc4c(c1)c23)c67 ; CC(C)(C)c1cc2c3cc4ccccc4c(-c4ccc5c(c4)c4ccccc4n5-c4ccccc4)c3n3c4cc5c6cc(C(C)(C)C)cc7c8cc9ccccc9c(-c9ccc%10c(c9)c9ccccc9n%10-c9ccccc9)c8n(c5nc4c(c1)c23)c67]

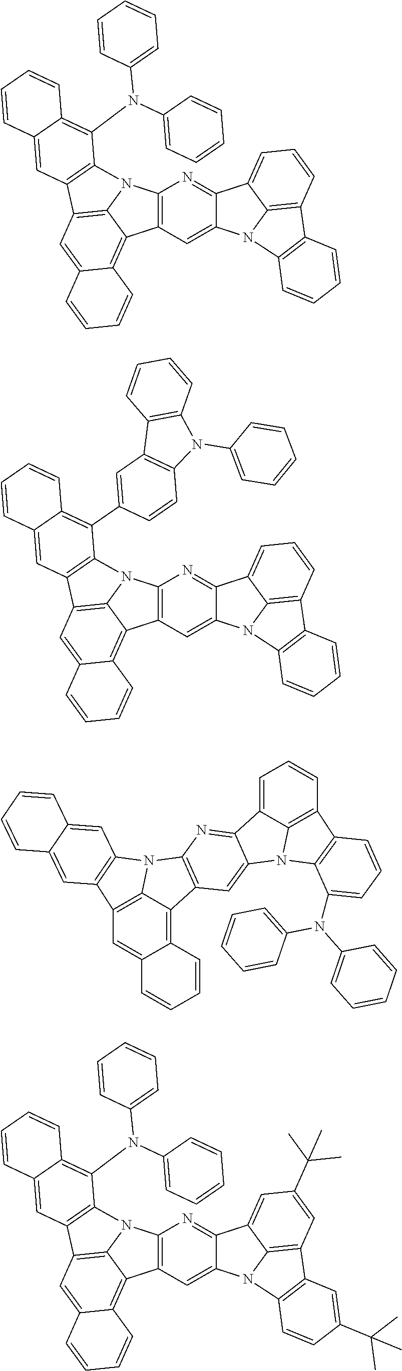

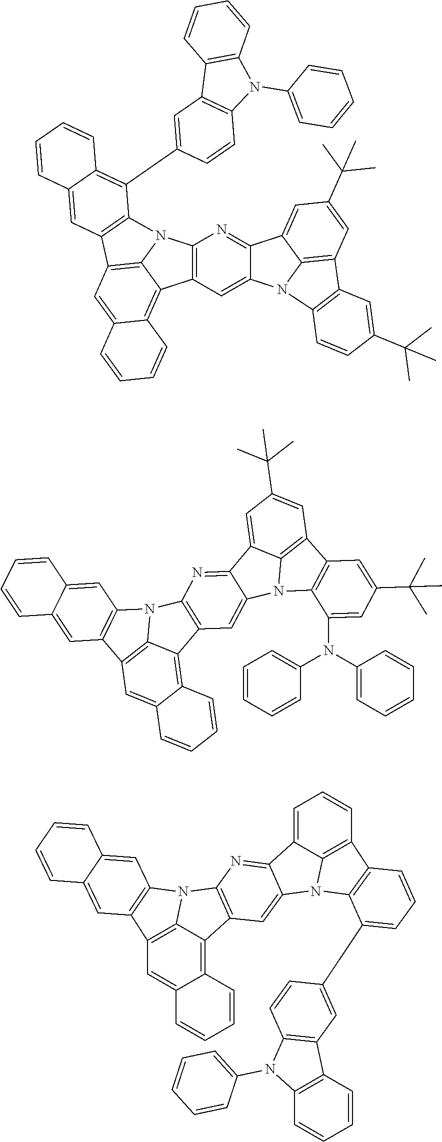 [SMILES: CC(C)(C)c1ccc2c(c1)c1cc(C(C)(C)C)cc3c4nc5c(cc4n2c13)c1c2ccccc2cc2c3cc4ccccc4c(-c4ccc6c(c4)c4ccccc4n6-c4ccccc4)c3n5c21 ; CC(C)(C)c1cc(N(c2ccccc2)c2ccccc2)c2c(c1)c1cc(C(C)(C)C)cc3c4nc5c(cc4n2c13)c1c2ccccc2cc2c3cc4ccccc4cc3n5c21 ; c1ccc(-n2c3ccccc3c3cc(-c4cccc5c6cccc7c8nc9c(cc8n(c45)c67)c4c5ccccc5cc5c6cc7ccccc7cc6n9c54)ccc32)cc1]

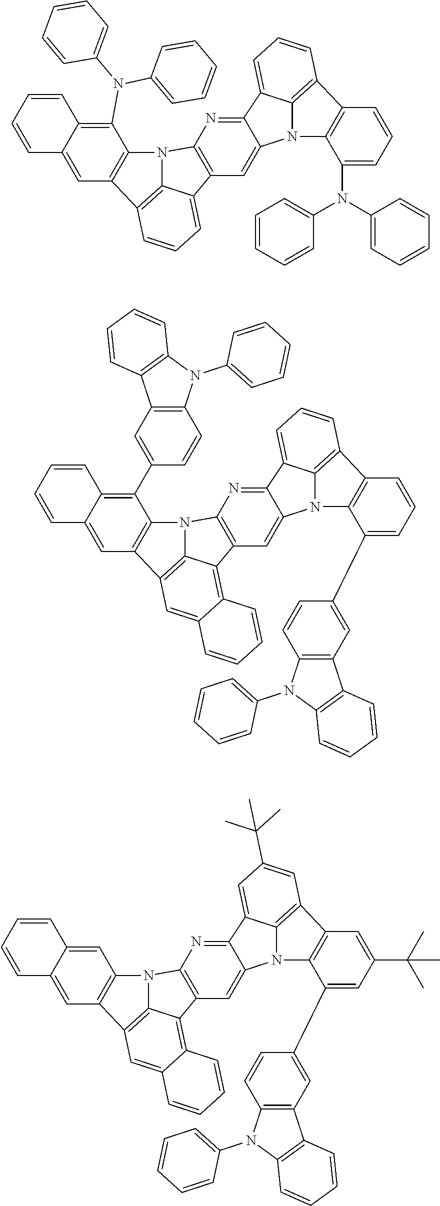 [SMILES: c1ccc(N(c2ccccc2)c2cccc3c4cccc5c6nc7c(cc6n(c23)c45)c2cccc3c4cc5ccccc5c(N(c5ccccc5)c5ccccc5)c4n7c23)cc1 ; c1ccc(-n2c3ccccc3c3cc(-c4cccc5c6cccc7c8nc9c(cc8n(c45)c67)c4c5ccccc5cc5c6cc7ccccc7c(-c7ccc8c(c7)c7ccccc7n8-c7ccccc7)c6n9c54)ccc32)cc1 ; CC(C)(C)c1cc(-c2ccc3c(c2)c2ccccc2n3-c2ccccc2)c2c(c1)c1cc(C(C)(C)C)cc3c4nc5c(cc4n2c13)c1c2ccccc2cc2c3cc4ccccc4cc3n5c21]

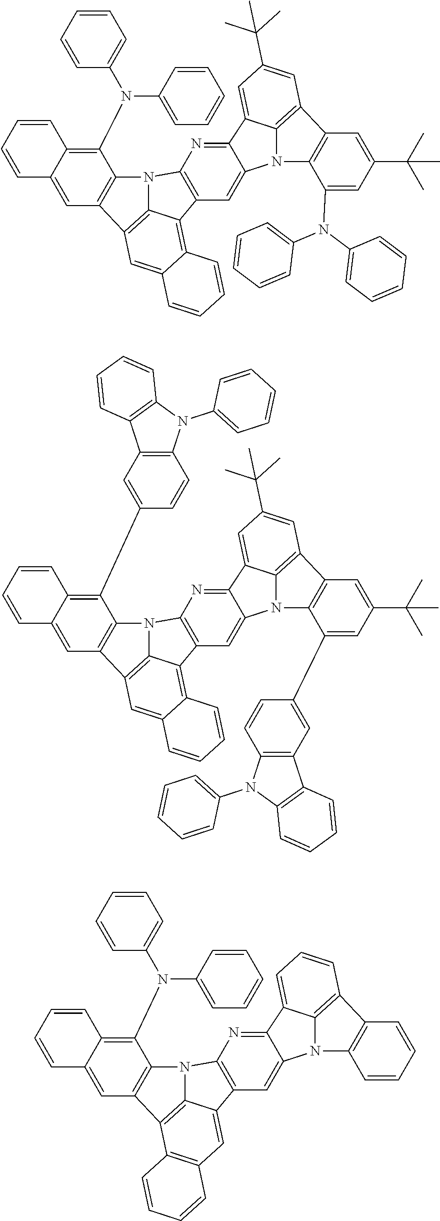 [SMILES: CC(C)(C)c1cc(N(c2ccccc2)c2ccccc2)c2c(c1)c1cc(C(C)(C)C)cc3c4nc5c(cc4n2c13)c1c2ccccc2cc2c3cc4ccccc4c(N(c4ccccc4)c4ccccc4)c3n5c21 ; CC(C)(C)c1cc(-c2ccc3c(c2)c2ccccc2n3-c2ccccc2)c2c(c1)c1cc(C(C)(C)C)cc3c4nc5c(cc4n2c13)c1c2ccccc2cc2c3cc4ccccc4c(-c4ccc6c(c4)c4ccccc4n6-c4ccccc4)c3n5c21 ; c1ccc(N(c2ccccc2)c2c3ccccc3cc3c4c5ccccc5cc5c6cc7c(nc6n(c23)c54)c2cccc3c4ccccc4n7c32)cc1]

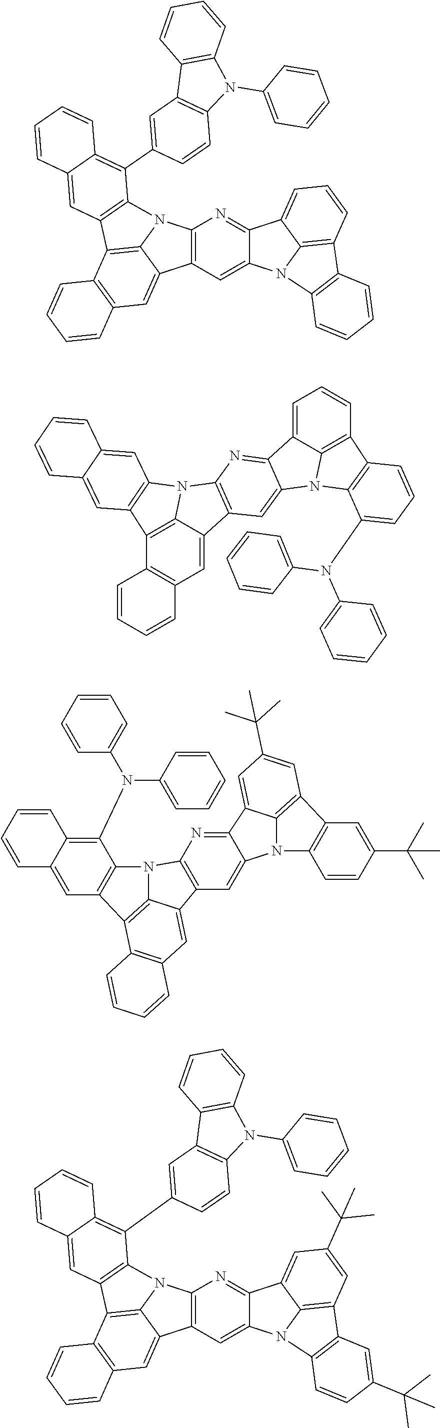 [SMILES: c1ccc(-n2c3ccccc3c3cc(-c4c5ccccc5cc5c6c7ccccc7cc7c8cc9c(nc8n(c45)c76)c4cccc5c6ccccc6n9c54)ccc32)cc1 ; c1ccc(N(c2ccccc2)c2cccc3c4cccc5c6nc7c(cc6n(c23)c45)c2cc3ccccc3c3c4cc5ccccc5cc4n7c23)cc1 ; CC(C)(C)c1ccc2c(c1)c1cc(C(C)(C)C)cc3c4nc5c(cc4n2c13)c1cc2ccccc2c2c3cc4ccccc4c(N(c4ccccc4)c4ccccc4)c3n5c12 ; CC(C)(C)c1ccc2c(c1)c1cc(C(C)(C)C)cc3c4nc5c(cc4n2c13)c1cc2ccccc2c2c3cc4ccccc4c(-c4ccc6c(c4)c4ccccc4n6-c4ccccc4)c3n5c12]

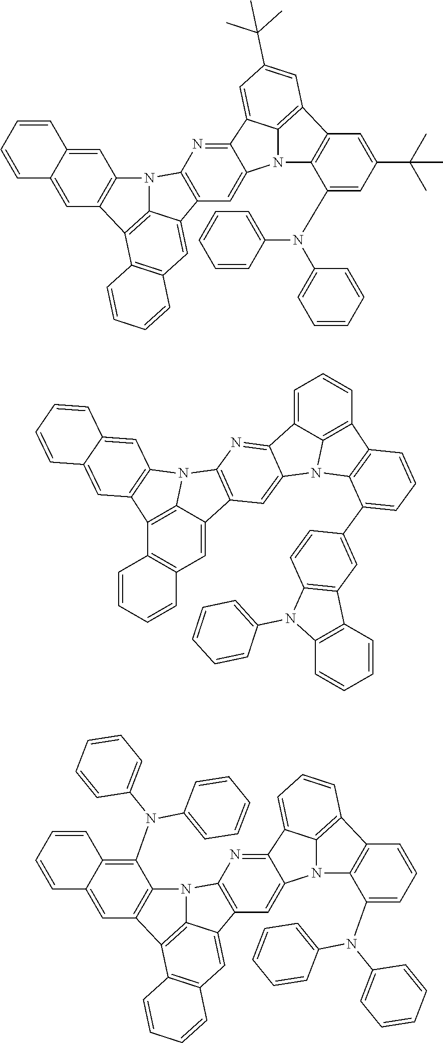 [SMILES: CC(C)(C)c1cc(N(c2ccccc2)c2ccccc2)c2c(c1)c1cc(C(C)(C)C)cc3c4nc5c(cc4n2c13)c1cc2ccccc2c2c3cc4ccccc4cc3n5c12 ; c1ccc(-n2c3ccccc3c3cc(-c4cccc5c6cccc7c8nc9c(cc8n(c45)c67)c4cc5ccccc5c5c6cc7ccccc7cc6n9c45)ccc32)cc1 ; c1ccc(N(c2ccccc2)c2cccc3c4cccc5c6nc7c(cc6n(c23)c45)c2cc3ccccc3c3c4cc5ccccc5c(N(c5ccccc5)c5ccccc5)c4n7c23)cc1]

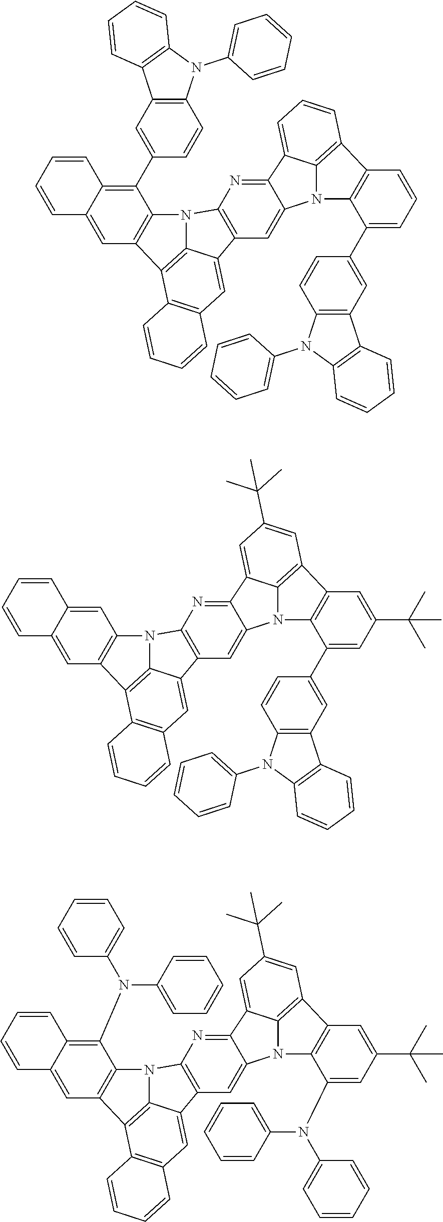 [SMILES: c1ccc(-n2c3ccccc3c3cc(-c4cccc5c6cccc7c8nc9c(cc8n(c45)c67)c4cc5ccccc5c5c6cc7ccccc7c(-c7ccc8c(c7)c7ccccc7n8-c7ccccc7)c6n9c45)ccc32)cc1 ; CC(C)(C)c1cc(-c2ccc3c(c2)c2ccccc2n3-c2ccccc2)c2c(c1)c1cc(C(C)(C)C)cc3c4nc5c(cc4n2c13)c1cc2ccccc2c2c3cc4ccccc4cc3n5c12 ; CC(C)(C)c1cc(N(c2ccccc2)c2ccccc2)c2c(c1)c1cc(C(C)(C)C)cc3c4nc5c(cc4n2c13)c1cc2ccccc2c2c3cc4ccccc4c(N(c4ccccc4)c4ccccc4)c3n5c12]

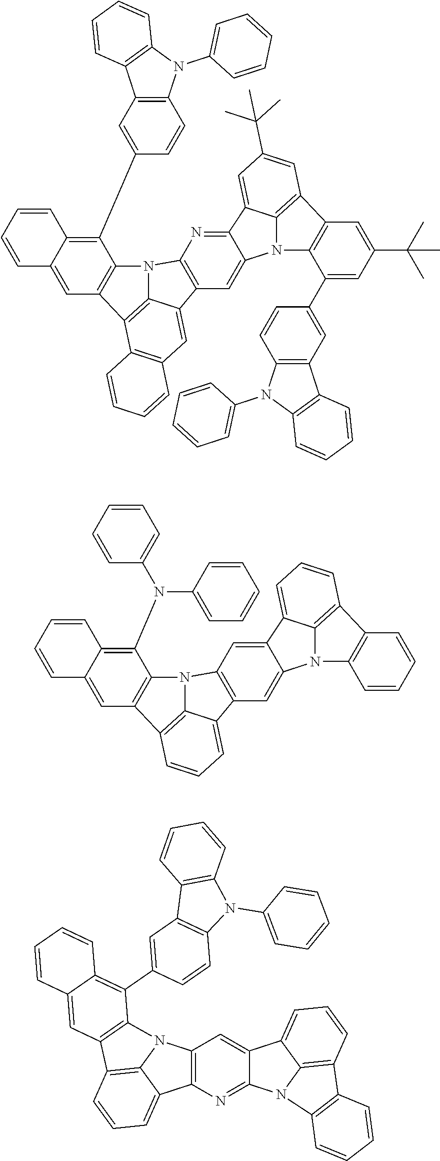

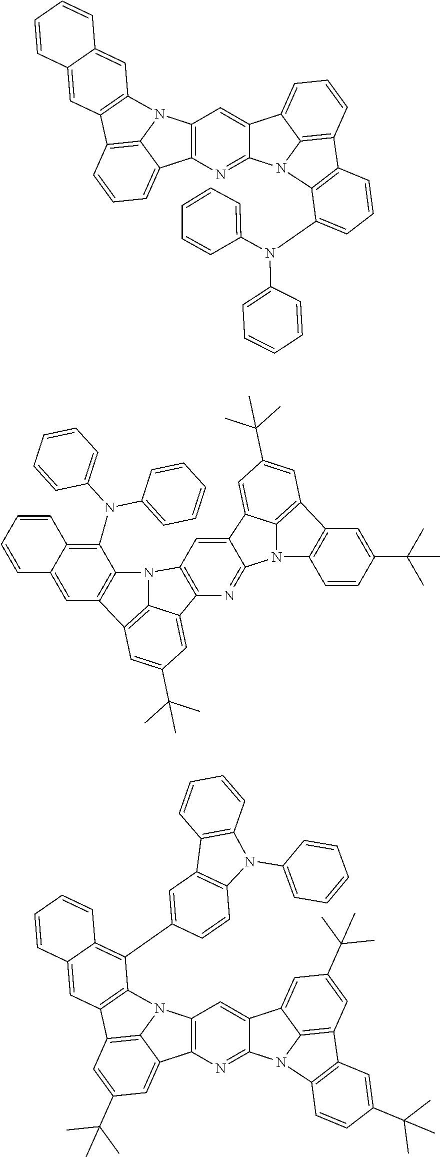 [SMILES: c1ccc(N(c2ccccc2)c2cccc3c4cccc5c6cc7c(nc6n(c23)c54)c2cccc3c4cc5ccccc5cc4n7c32)cc1 ; CC(C)(C)c1ccc2c(c1)c1cc(C(C)(C)C)cc3c4cc5c(nc4n2c13)c1cc(C(C)(C)C)cc2c3cc4ccccc4c(N(c4ccccc4)c4ccccc4)c3n5c21 ; CC(C)(C)c1ccc2c(c1)c1cc(C(C)(C)C)cc3c4cc5c(nc4n2c13)c1cc(C(C)(C)C)cc2c3cc4ccccc4c(-c4ccc6c(c4)c4ccccc4n6-c4ccccc4)c3n5c21]

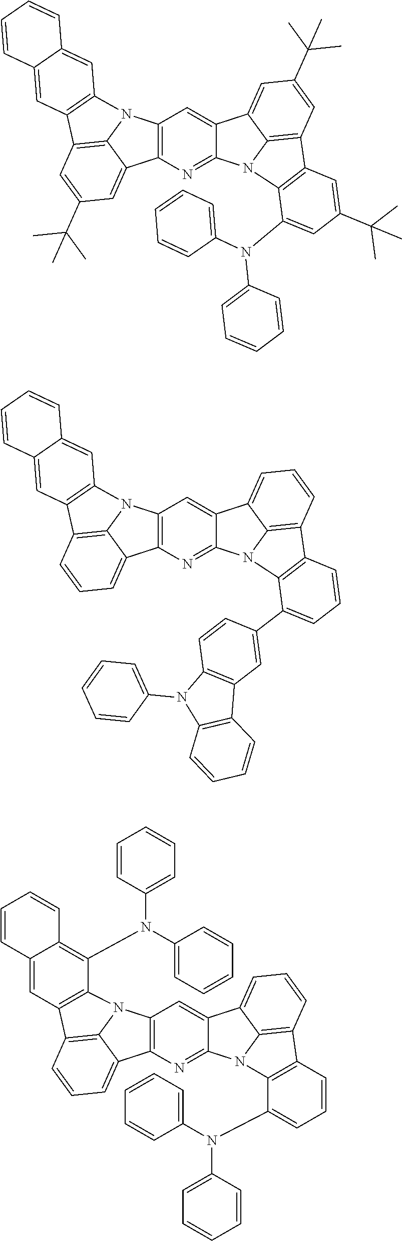 [SMILES: CC(C)(C)c1cc(N(c2ccccc2)c2ccccc2)c2c(c1)c1cc(C(C)(C)C)cc3c4cc5c(nc4n2c31)c1cc(C(C)(C)C)cc2c3cc4ccccc4cc3n5c21 ; c1ccc(-n2c3ccccc3c3cc(-c4cccc5c6cccc7c8cc9c(nc8n(c45)c76)c4cccc5c6cc7ccccc7cc6n9c54)ccc32)cc1 ; c1ccc(N(c2ccccc2)c2c3ccccc3cc3c4cccc5c6nc7c(cc6n(c23)c45)c2cccc3c4cccc(N(c5ccccc5)c5ccccc5)c4n7c23)cc1]

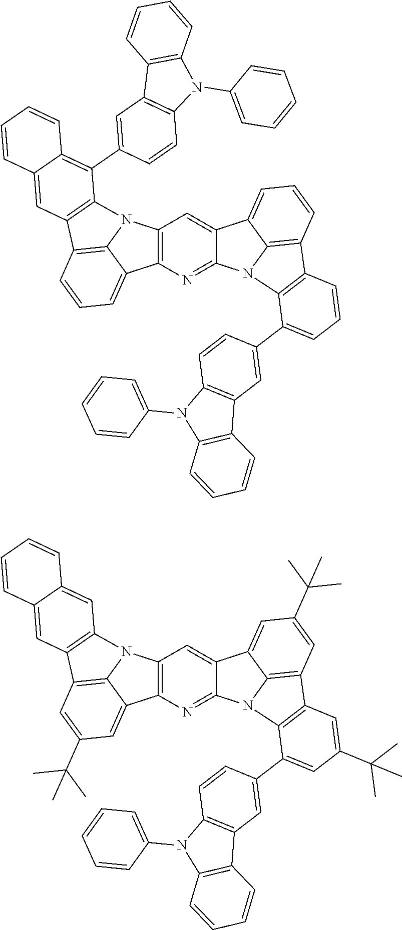 [SMILES: c1ccc(-n2c3ccccc3c3cc(-c4c5ccccc5cc5c6cccc7c8nc9c(cc8n(c45)c67)c4cccc5c6cccc(-c7ccc8c(c7)c7ccccc7n8-c7ccccc7)c6n9c45)ccc32)cc1 ; CC(C)(C)c1cc(-c2ccc3c(c2)c2ccccc2n3-c2ccccc2)c2c(c1)c1cc(C(C)(C)C)cc3c4cc5c(nc4n2c31)c1cc(C(C)(C)C)cc2c3cc4ccccc4cc3n5c21]

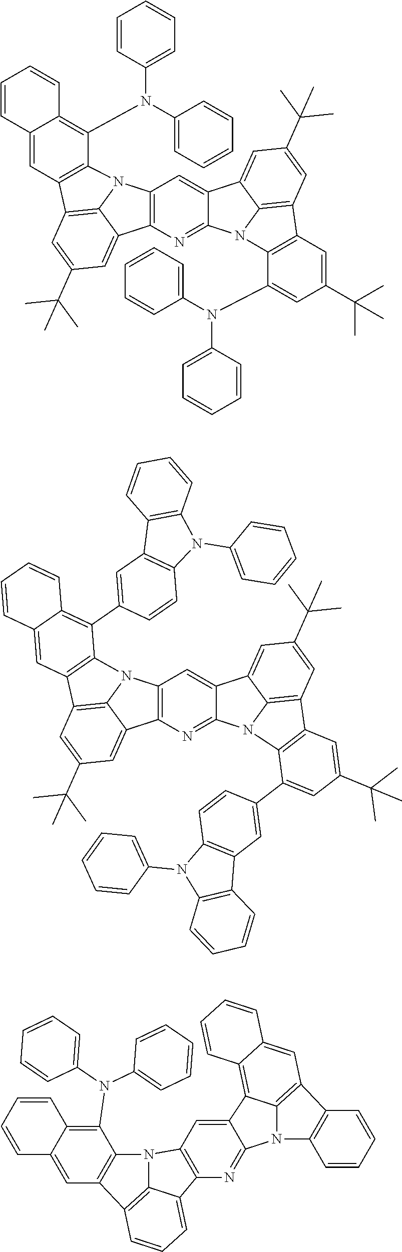 [SMILES: CC(C)(C)c1cc(N(c2ccccc2)c2ccccc2)c2c(c1)c1cc(C(C)(C)C)cc3c4cc5c(nc4n2c31)c1cc(C(C)(C)C)cc2c3cc4ccccc4c(N(c4ccccc4)c4ccccc4)c3n5c21 ; CC(C)(C)c1cc(-c2ccc3c(c2)c2ccccc2n3-c2ccccc2)c2c(c1)c1cc(C(C)(C)C)cc3c4cc5c(nc4n2c31)c1cc(C(C)(C)C)cc2c3cc4ccccc4c(-c4ccc6c(c4)c4ccccc4n6-c4ccccc4)c3n5c21 ; c1ccc(N(c2ccccc2)c2c3ccccc3cc3c4cccc5c6nc7c(cc6n(c23)c45)c2c3ccccc3cc3c4ccccc4n7c32)cc1]

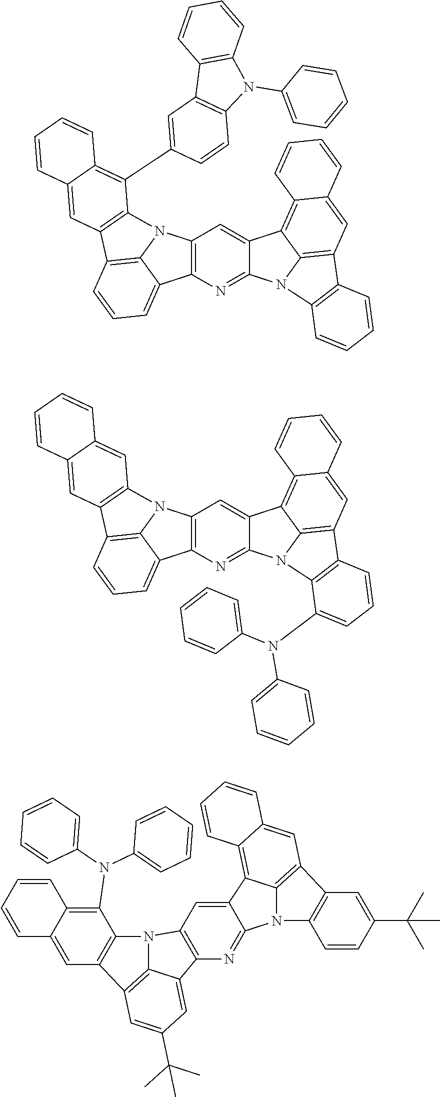 [SMILES: c1ccc(-n2c3ccccc3c3cc(-c4c5ccccc5cc5c6cccc7c8nc9c(cc8n(c45)c67)c4c5ccccc5cc5c6ccccc6n9c54)ccc32)cc1 ; c1ccc(N(c2ccccc2)c2cccc3c4cc5ccccc5c5c6cc7c(nc6n(c23)c45)c2cccc3c4cc5ccccc5cc4n7c32)cc1 ; CC(C)(C)c1ccc2c(c1)c1cc3ccccc3c3c4cc5c(nc4n2c13)c1cc(C(C)(C)C)cc2c3cc4ccccc4c(N(c4ccccc4)c4ccccc4)c3n5c21]

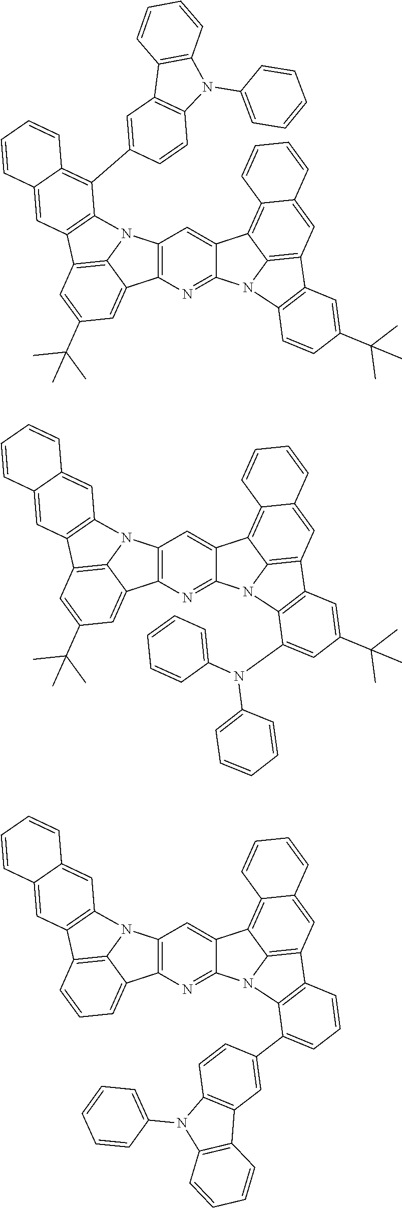 [SMILES: CC(C)(C)c1ccc2c(c1)c1cc3ccccc3c3c4cc5c(nc4n2c13)c1cc(C(C)(C)C)cc2c3cc4ccccc4c(-c4ccc6c(c4)c4ccccc4n6-c4ccccc4)c3n5c21 ; CC(C)(C)c1cc(N(c2ccccc2)c2ccccc2)c2c(c1)c1cc3ccccc3c3c4cc5c(nc4n2c13)c1cc(C(C)(C)C)cc2c3cc4ccccc4cc3n5c21 ; c1ccc(-n2c3ccccc3c3cc(-c4cccc5c6cc7ccccc7c7c8cc9c(nc8n(c45)c67)c4cccc5c6cc7ccccc7cc6n9c54)ccc32)cc1]

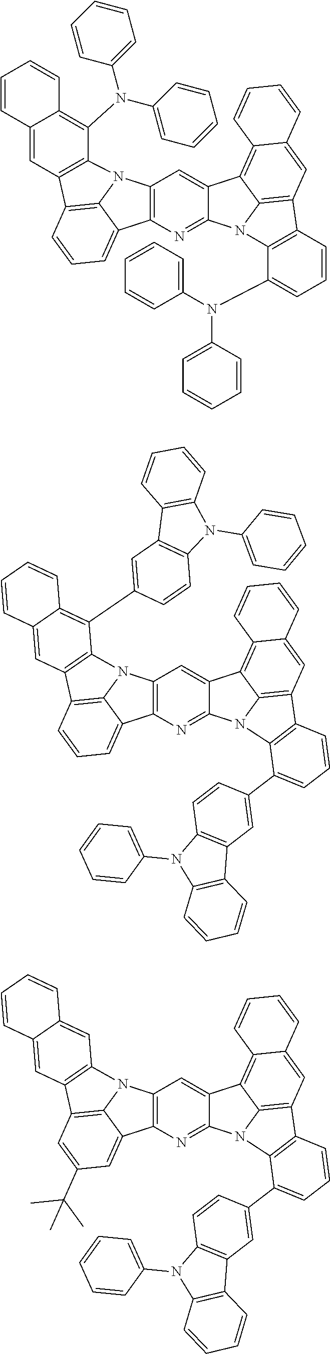 [SMILES: c1ccc(N(c2ccccc2)c2c3ccccc3cc3c4cccc5c6nc7c(cc6n(c23)c45)c2c3ccccc3cc3c4cccc(N(c5ccccc5)c5ccccc5)c4n7c32)cc1 ; c1ccc(-n2c3ccccc3c3cc(-c4c5ccccc5cc5c6cccc7c8nc9c(cc8n(c45)c67)c4c5ccccc5cc5c6cccc(-c7ccc8c(c7)c7ccccc7n8-c7ccccc7)c6n9c54)ccc32)cc1 ; CC(C)(C)c1cc2c3cc4ccccc4cc3n3c4cc5c6c7ccccc7cc7c8cccc(-c9ccc%10c(c9)c9ccccc9n%10-c9ccccc9)c8n(c5nc4c(c1)c23)c76]

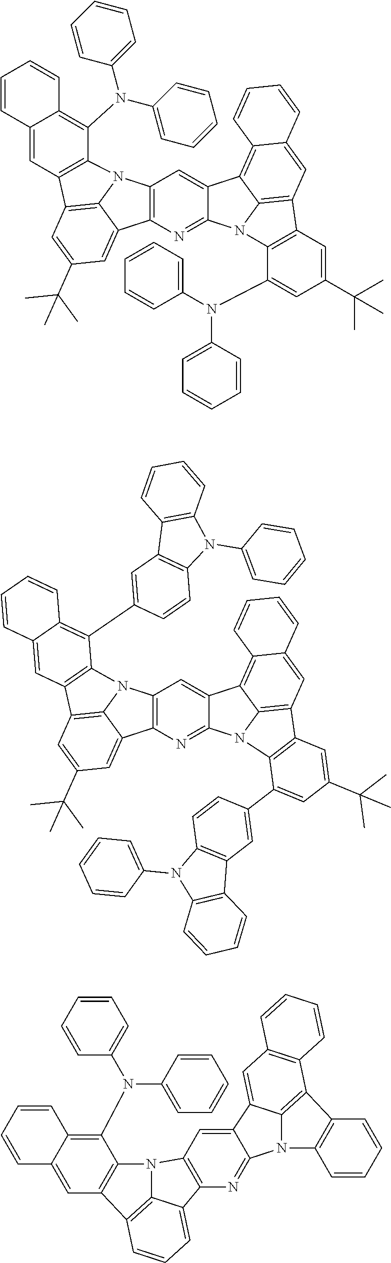 [SMILES: CC(C)(C)c1cc(N(c2ccccc2)c2ccccc2)c2c(c1)c1cc3ccccc3c3c4cc5c(nc4n2c13)c1cc(C(C)(C)C)cc2c3cc4ccccc4c(N(c4ccccc4)c4ccccc4)c3n5c21 ; CC(C)(C)c1cc(-c2ccc3c(c2)c2ccccc2n3-c2ccccc2)c2c(c1)c1cc3ccccc3c3c4cc5c(nc4n2c13)c1cc(C(C)(C)C)cc2c3cc4ccccc4c(-c4ccc6c(c4)c4ccccc4n6-c4ccccc4)c3n5c21 ; c1ccc(N(c2ccccc2)c2c3ccccc3cc3c4cccc5c6nc7c(cc6n(c23)c45)c2cc3ccccc3c3c4ccccc4n7c23)cc1]

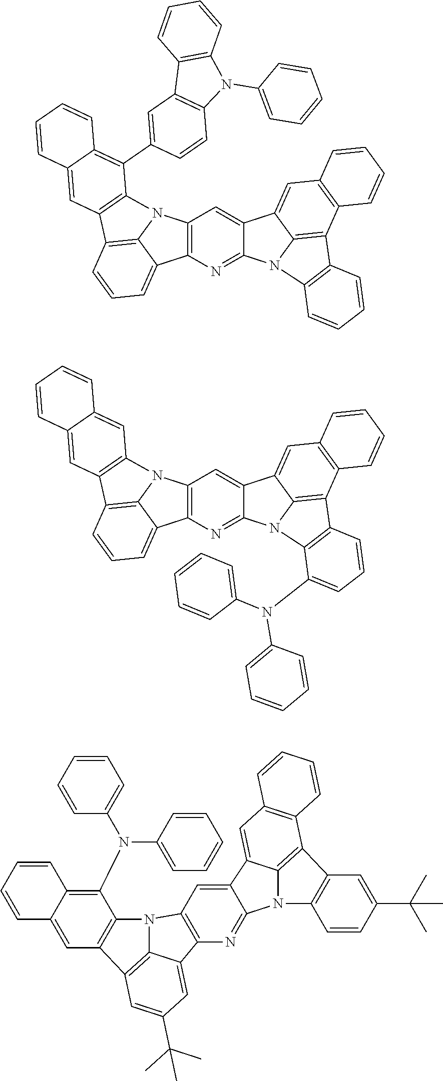

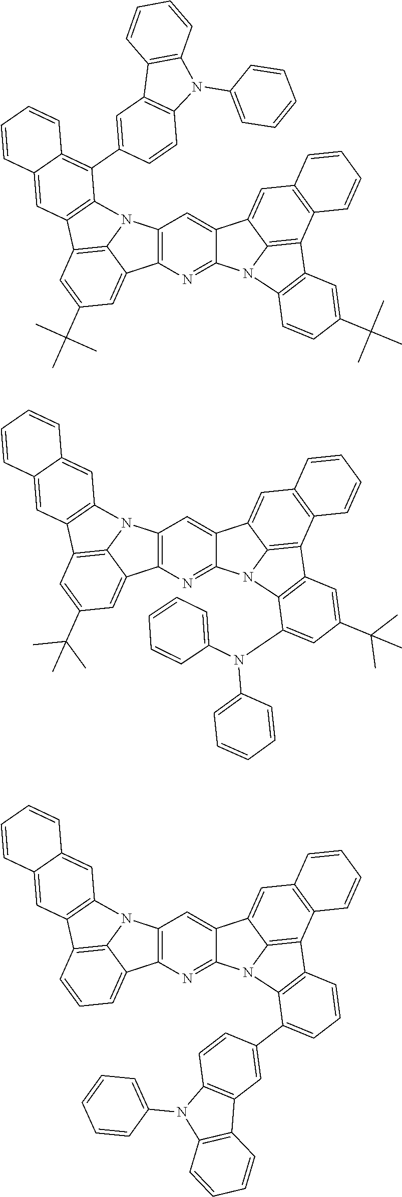 [SMILES: CC(C)(C)c1ccc2c(c1)c1c3ccccc3cc3c4cc5c(nc4n2c31)c1cc(C(C)(C)C)cc2c3cc4ccccc4c(-c4ccc6c(c4)c4ccccc4n6-c4ccccc4)c3n5c21 ; CC(C)(C)c1cc(N(c2ccccc2)c2ccccc2)c2c(c1)c1c3ccccc3cc3c4cc5c(nc4n2c31)c1cc(C(C)(C)C)cc2c3cc4ccccc4cc3n5c21 ; c1ccc(-n2c3ccccc3c3cc(-c4cccc5c6c7ccccc7cc7c8cc9c(nc8n(c45)c76)c4cccc5c6cc7ccccc7cc6n9c54)ccc32)cc1]

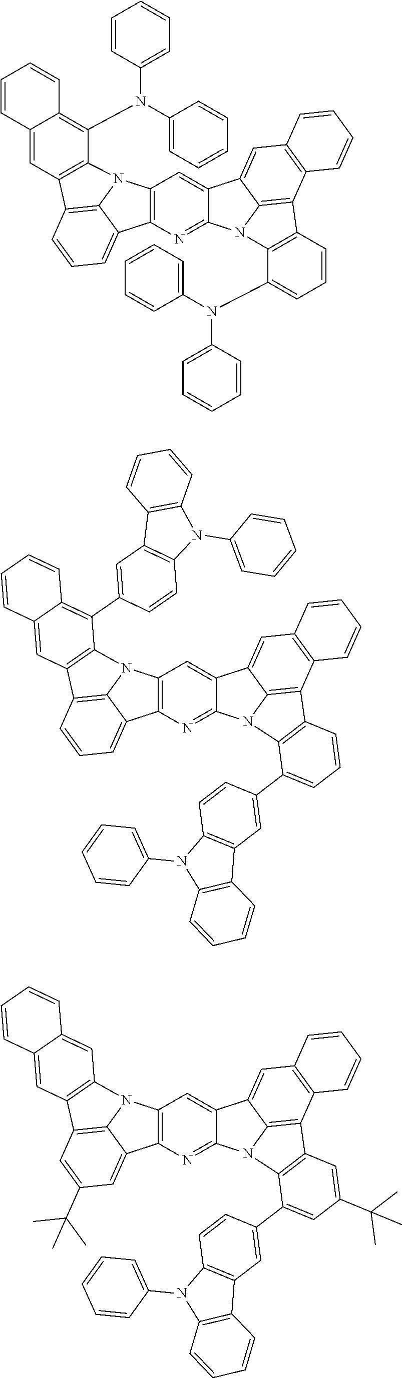 [SMILES: c1ccc(N(c2ccccc2)c2c3ccccc3cc3c4cccc5c6nc7c(cc6n(c23)c45)c2cc3ccccc3c3c4cccc(N(c5ccccc5)c5ccccc5)c4n7c23)cc1 ; c1ccc(-n2c3ccccc3c3cc(-c4c5ccccc5cc5c6cccc7c8nc9c(cc8n(c45)c67)c4cc5ccccc5c5c6cccc(-c7ccc8c(c7)c7ccccc7n8-c7ccccc7)c6n9c45)ccc32)cc1 ; CC(C)(C)c1cc(-c2ccc3c(c2)c2ccccc2n3-c2ccccc2)c2c(c1)c1c3ccccc3cc3c4cc5c(nc4n2c31)c1cc(C(C)(C)C)cc2c3cc4ccccc4cc3n5c21]

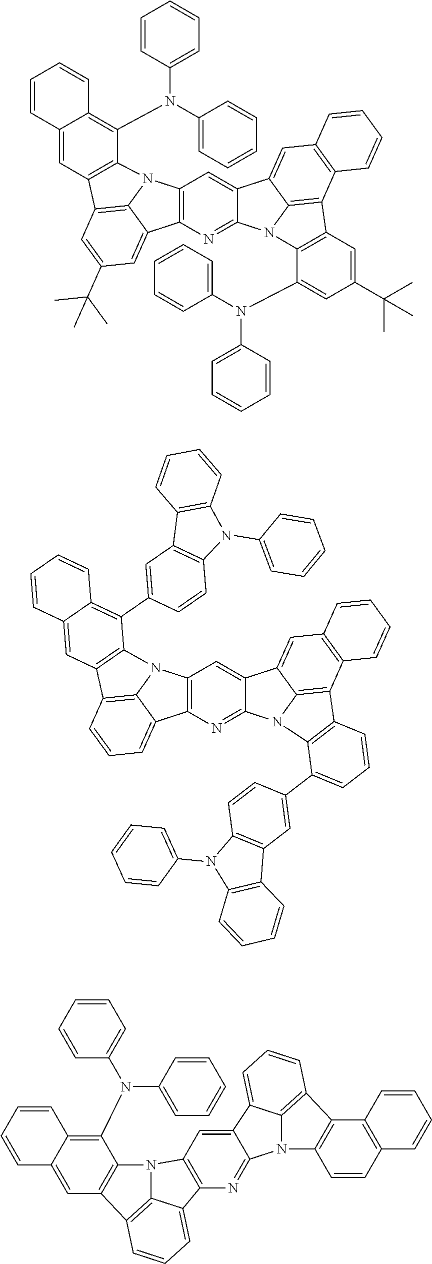 [SMILES: CC(C)(C)c1cc(N(c2ccccc2)c2ccccc2)c2c(c1)c1c3ccccc3cc3c4cc5c(nc4n2c31)c1cc(C(C)(C)C)cc2c3cc4ccccc4c(N(c4ccccc4)c4ccccc4)c3n5c21 ; c1ccc(-n2c3ccccc3c3cc(-c4c5ccccc5cc5c6cccc7c8nc9c(cc8n(c45)c67)c4cc5ccccc5c5c6cccc(-c7ccc8c(c7)c7ccccc7n8-c7ccccc7)c6n9c45)ccc32)cc1 ; c1ccc(N(c2ccccc2)c2c3ccccc3cc3c4cccc5c6nc7c(cc6n(c23)c45)c2cccc3c4c5ccccc5ccc4n7c23)cc1]

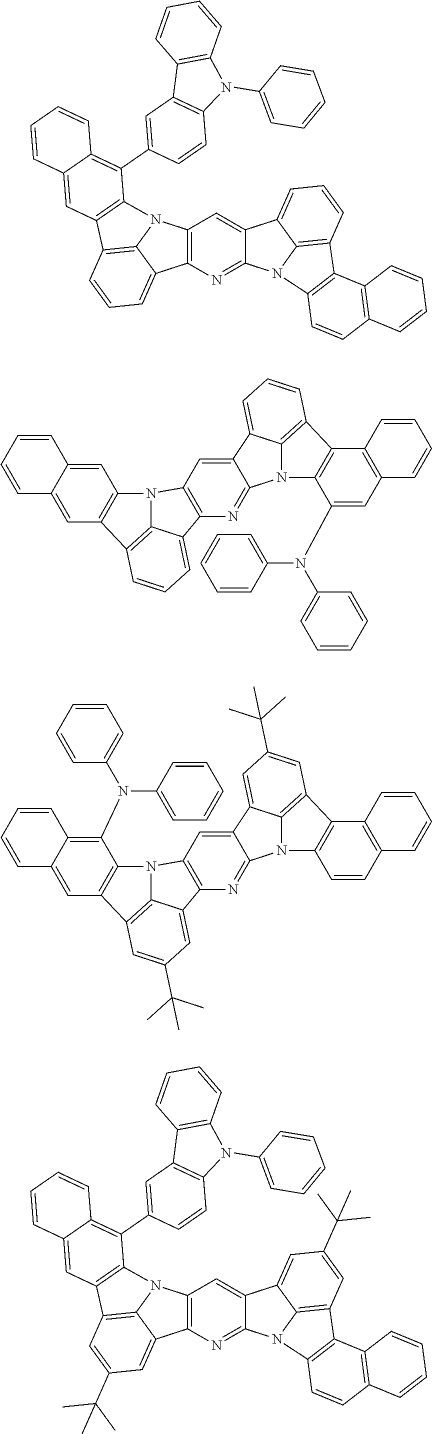

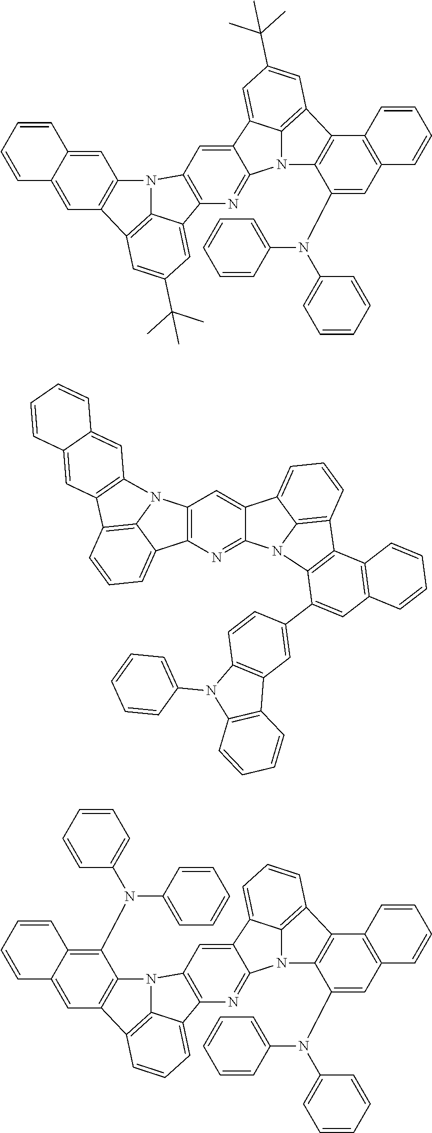 [SMILES: CC(C)(C)c1cc2c3cc4ccccc4cc3n3c4cc5c6cc(C(C)(C)C)cc7c8c9ccccc9cc(N(c9ccccc9)c9ccccc9)c8n(c5nc4c(c1)c23)c67 ; c1ccc(-n2c3ccccc3c3cc(-c4cc5ccccc5c5c6cccc7c8cc9c(nc8n(c45)c76)c4cccc5c6cc7ccccc7cc6n9c54)ccc32)cc1 ; c1ccc(N(c2ccccc2)c2c3ccccc3cc3c4cccc5c6nc7c(cc6n(c23)c45)c2cccc3c4c5ccccc5cc(N(c5ccccc5)c5ccccc5)c4n7c23)cc1]

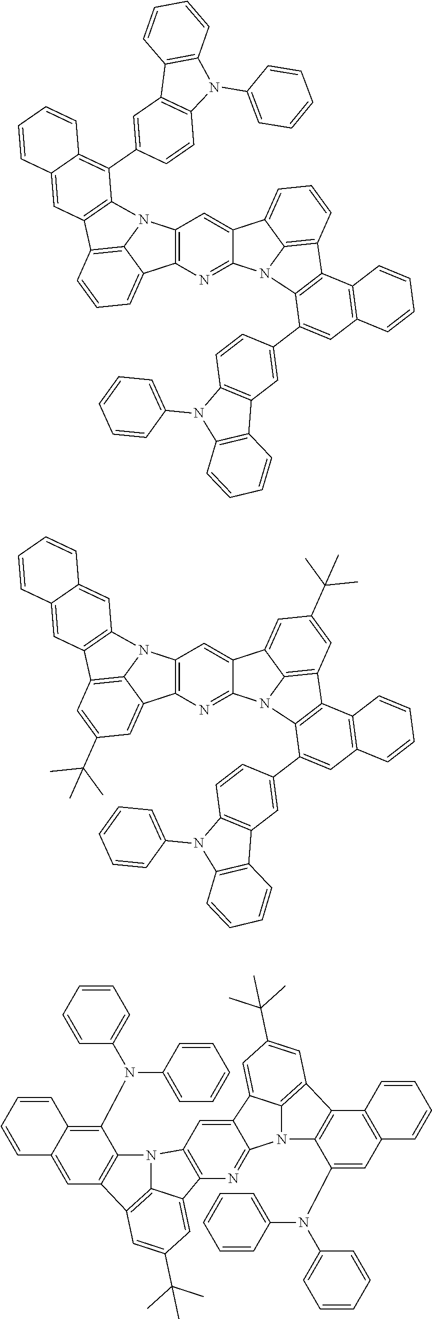 [SMILES: c1ccc(-n2c3ccccc3c3cc(-c4c5ccccc5cc5c6cccc7c8nc9c(cc8n(c45)c67)c4cccc5c6c7ccccc7cc(-c7ccc8c(c7)c7ccccc7n8-c7ccccc7)c6n9c45)ccc32)cc1 ; CC(C)(C)c1cc2c3cc4ccccc4cc3n3c4cc5c6cc(C(C)(C)C)cc7c8c9ccccc9cc(-c9ccc%10c(c9)c9ccccc9n%10-c9ccccc9)c8n(c5nc4c(c1)c23)c67 ; CC(C)(C)c1cc2c3cc4ccccc4c(N(c4ccccc4)c4ccccc4)c3n3c4cc5c6cc(C(C)(C)C)cc7c8c9ccccc9cc(N(c9ccccc9)c9ccccc9)c8n(c5nc4c(c1)c23)c67]

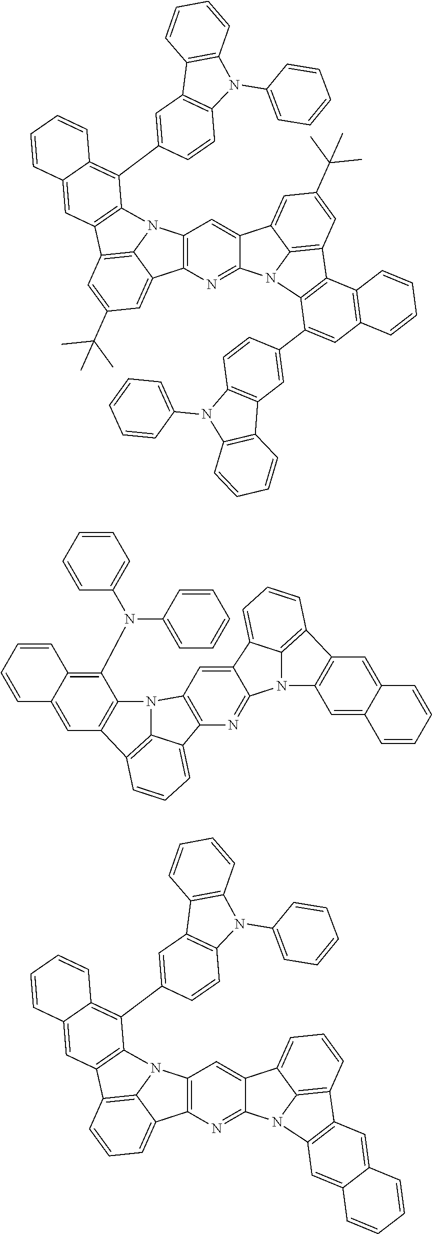

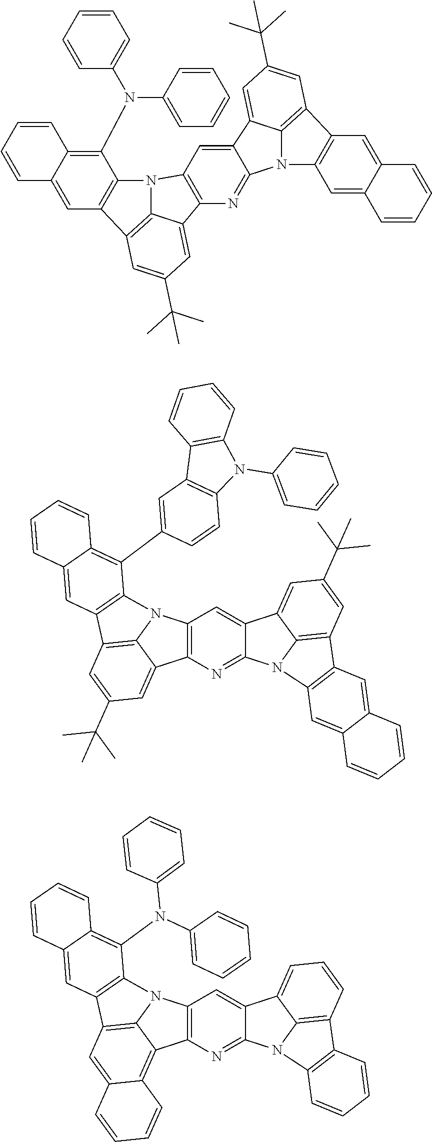

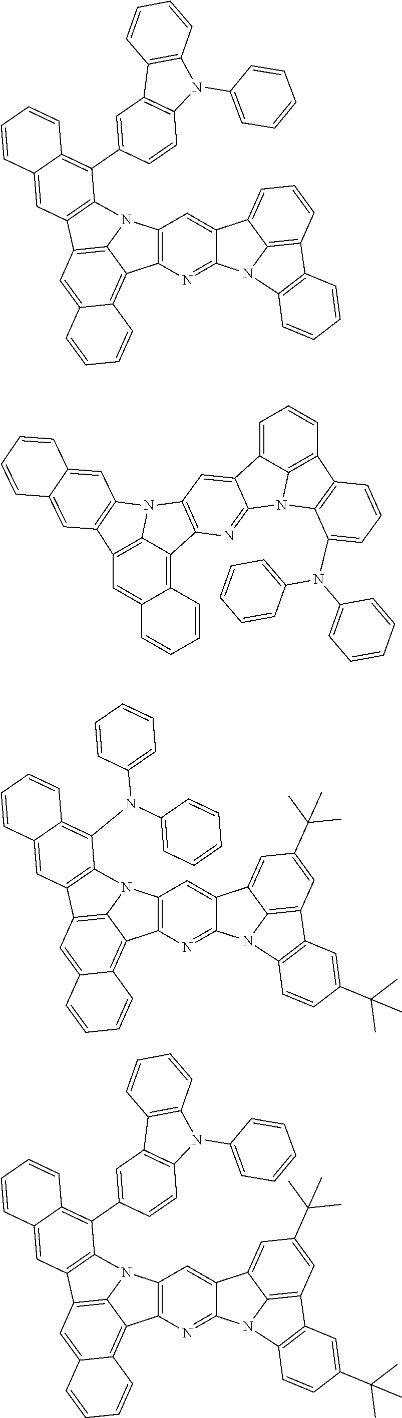

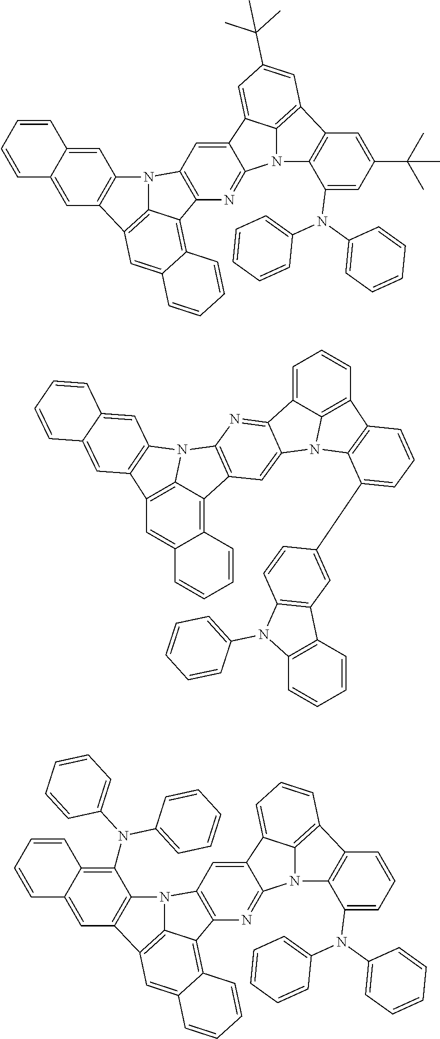 [SMILES: CC(C)(C)c1cc(N(c2ccccc2)c2ccccc2)c2c(c1)c1cc(C(C)(C)C)cc3c4cc5c(nc4n2c31)c1c2ccccc2cc2c3cc4ccccc4cc3n5c21 ; c1ccc(-n2c3ccccc3c3cc(-c4cccc5c6cccc7c8nc9c(cc8n(c45)c67)c4c5ccccc5cc5c6cc7ccccc7cc6n9c54)ccc32)cc1 ; c1ccc(N(c2ccccc2)c2c3ccccc3cc3c4cc5ccccc5c5c6nc7c(cc6n(c23)c45)c2cccc3c4cccc(N(c5ccccc5)c5ccccc5)c4n7c23)cc1]

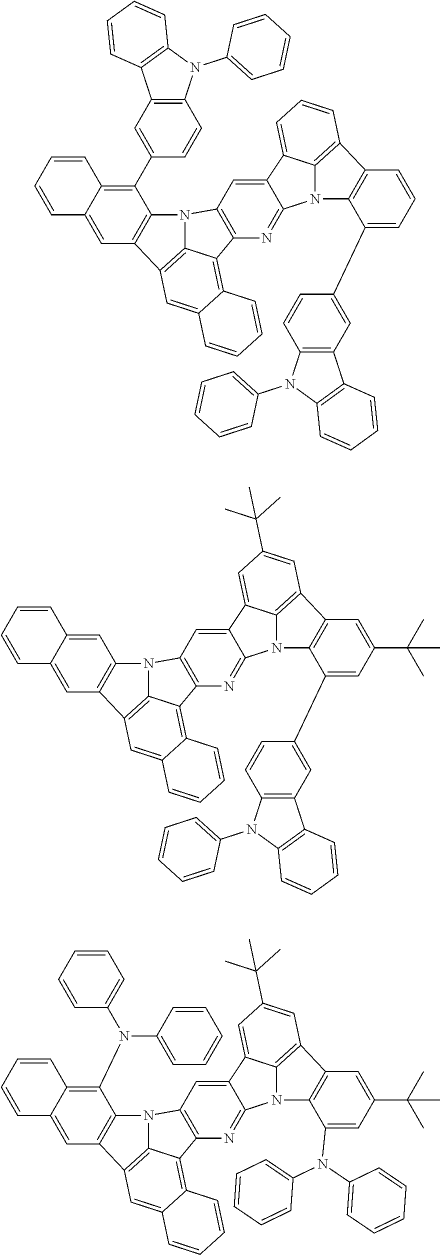 [SMILES: c1ccc(-n2c3ccccc3c3cc(-c4c5ccccc5cc5c6cc7ccccc7c7c8nc9c(cc8n(c45)c67)c4cccc5c6cccc(-c7ccc8c(c7)c7ccccc7n8-c7ccccc7)c6n9c45)ccc32)cc1 ; CC(C)(C)c1cc(-c2ccc3c(c2)c2ccccc2n3-c2ccccc2)c2c(c1)c1cc(C(C)(C)C)cc3c4cc5c(nc4n2c31)c1c2ccccc2cc2c3cc4ccccc4cc3n5c21 ; CC(C)(C)c1cc(N(c2ccccc2)c2ccccc2)c2c(c1)c1cc(C(C)(C)C)cc3c4cc5c(nc4n2c31)c1c2ccccc2cc2c3cc4ccccc4c(N(c4ccccc4)c4ccccc4)c3n5c21]

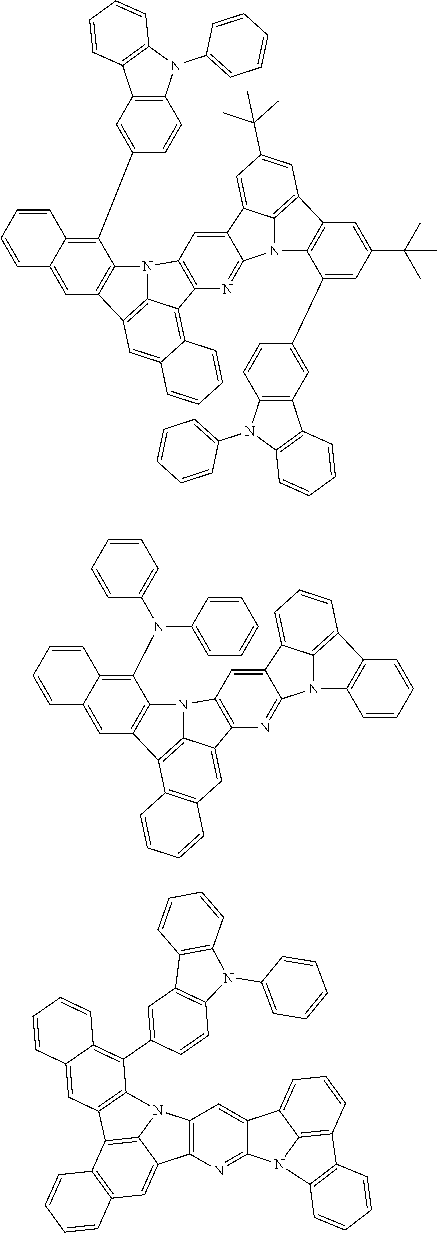

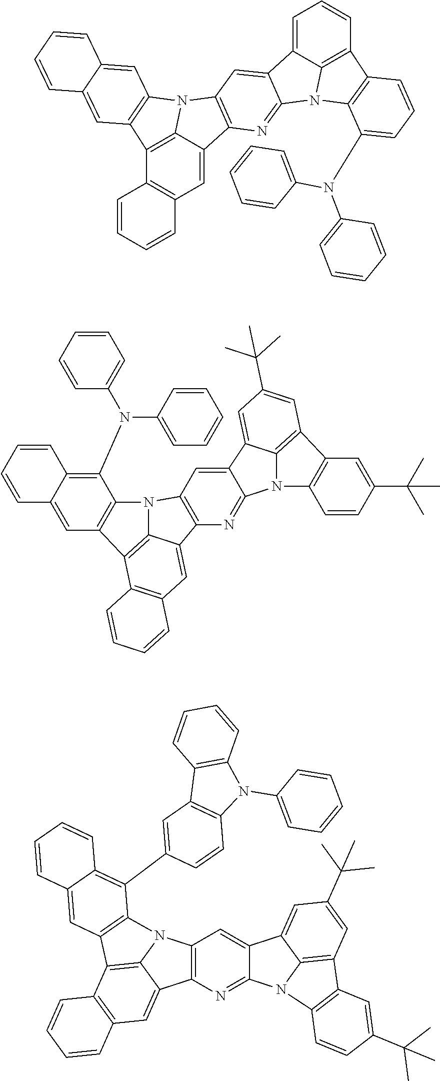 [SMILES: c1ccc(N(c2ccccc2)c2cccc3c4cccc5c6cc7c(nc6n(c23)c54)c2cc3ccccc3c3c4cc5ccccc5cc4n7c23)cc1 ; CC(C)(C)c1ccc2c(c1)c1cc(C(C)(C)C)cc3c4cc5c(nc4n2c13)c1cc2ccccc2c2c3cc4ccccc4c(N(c4ccccc4)c4ccccc4)c3n5c12 ; CC(C)(C)c1ccc2c(c1)c1cc(C(C)(C)C)cc3c4cc5c(nc4n2c13)c1cc2ccccc2c2c3cc4ccccc4c(-c4ccc6c(c4)c4ccccc4n6-c4ccccc4)c3n5c12]

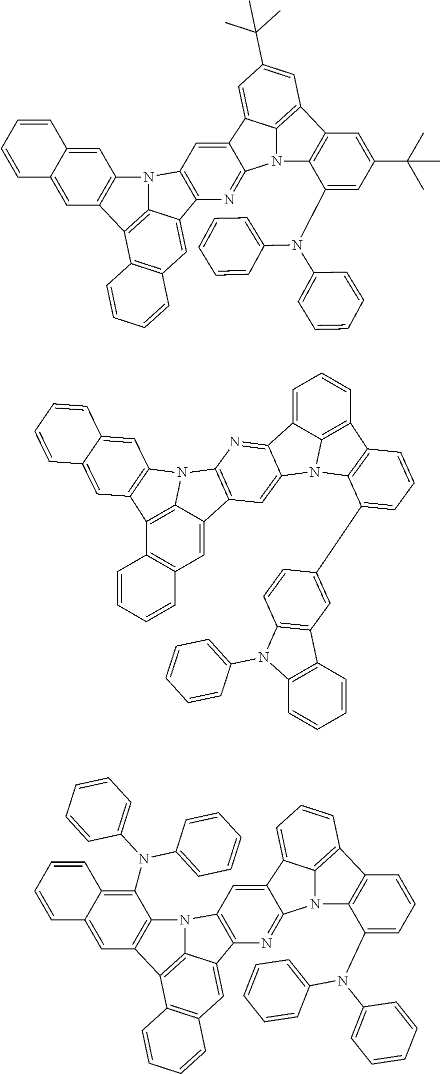

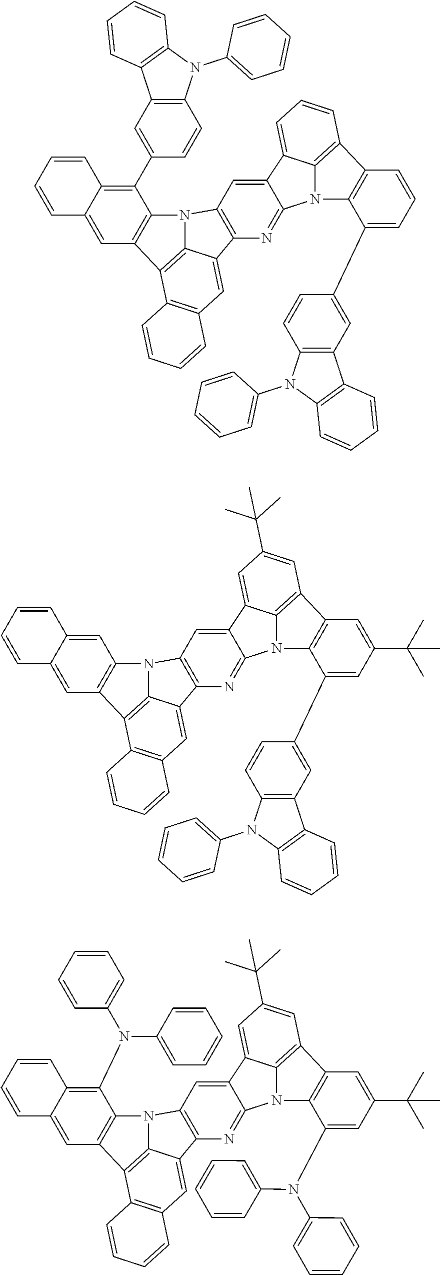 [SMILES: c1ccc(-n2c3ccccc3c3cc(-c4c5ccccc5cc5c6c7ccccc7cc7c8nc9c(cc8n(c45)c76)c4cccc5c6cccc(-c7ccc8c(c7)c7ccccc7n8-c7ccccc7)c6n9c45)ccc32)cc1 ; CC(C)(C)c1cc(-c2ccc3c(c2)c2ccccc2n3-c2ccccc2)c2c(c1)c1cc(C(C)(C)C)cc3c4cc5c(nc4n2c31)c1cc2ccccc2c2c3cc4ccccc4cc3n5c12 ; CC(C)(C)c1cc(N(c2ccccc2)c2ccccc2)c2c(c1)c1cc(C(C)(C)C)cc3c4cc5c(nc4n2c31)c1cc2ccccc2c2c3cc4ccccc4c(N(c4ccccc4)c4ccccc4)c3n5c12]

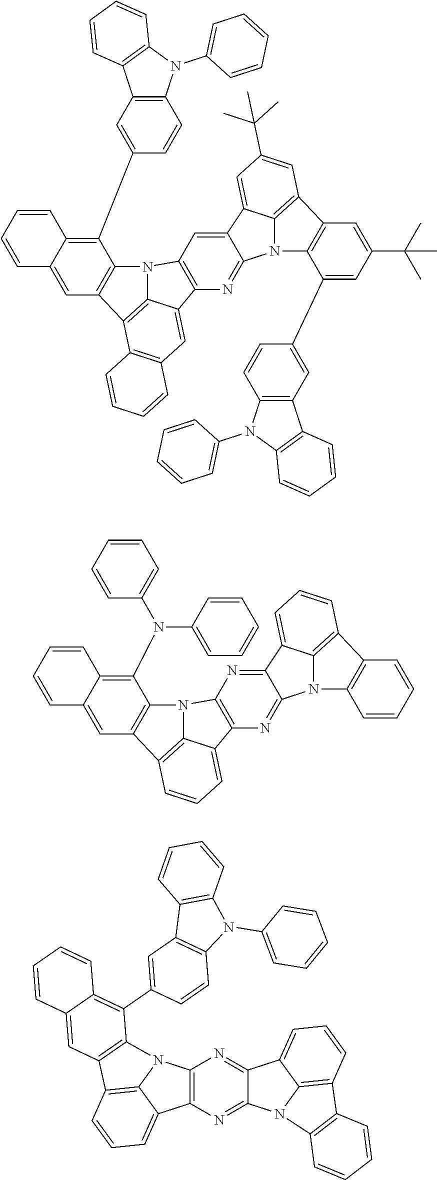

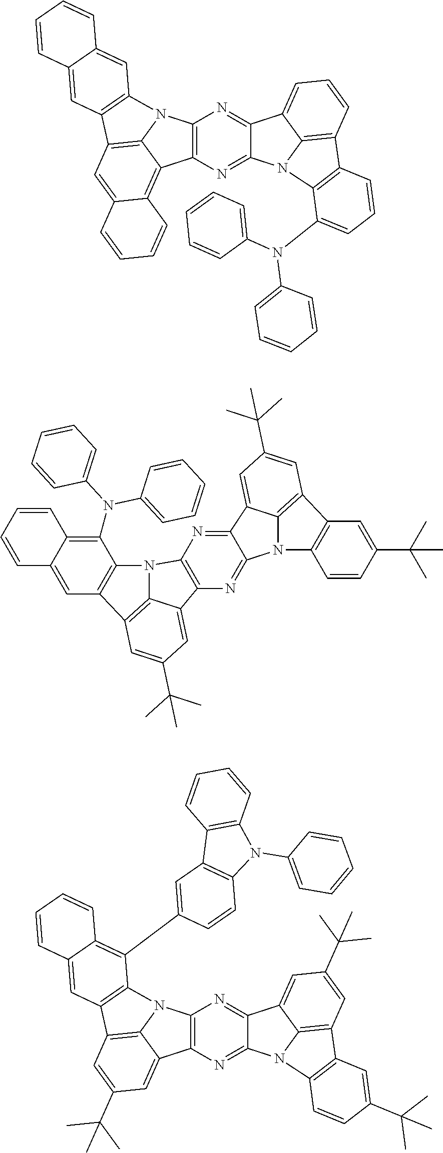 [SMILES: c1ccc(N(c2ccccc2)c2cccc3c4cccc5c6nc7c(nc6n(c23)c45)c2c3ccccc3cc3c4cc5ccccc5cc4n7c32)cc1 ; CC(C)(C)c1ccc2c(c1)c1cc(C(C)(C)C)cc3c4nc5c(nc4n2c13)c1cc(C(C)(C)C)cc2c3cc4ccccc4c(N(c4ccccc4)c4ccccc4)c3n5c21 ; CC(C)(C)c1ccc2c(c1)c1cc(C(C)(C)C)cc3c4nc5c(nc4n2c13)c1cc(C(C)(C)C)cc2c3cc4ccccc4c(-c4ccc6c(c4)c4ccccc4n6-c4ccccc4)c3n5c21]

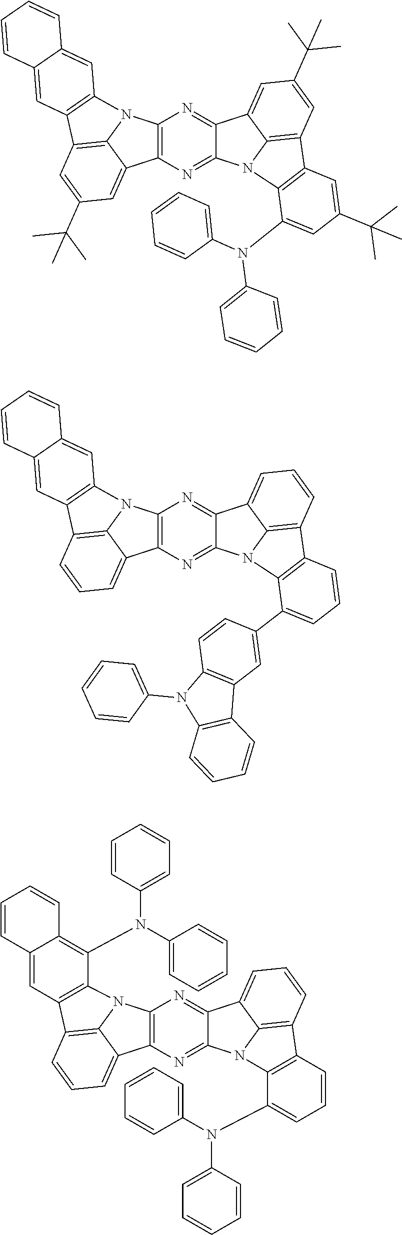 [SMILES: CC(C)(C)c1cc(N(c2ccccc2)c2ccccc2)c2c(c1)c1cc(C(C)(C)C)cc3c4nc5c(nc4n2c13)c1cc(C(C)(C)C)cc2c3cc4ccccc4cc3n5c21 ; c1ccc(-n2c3ccccc3c3cc(-c4cccc5c6cccc7c8nc9c(nc8n(c45)c67)c4cccc5c6cc7ccccc7cc6n9c54)ccc32)cc1 ; c1ccc(N(c2ccccc2)c2cccc3c4cccc5c6nc7c(nc6n(c23)c45)c2cccc3c4cc5ccccc5c(N(c5ccccc5)c5ccccc5)c4n7c32)cc1]

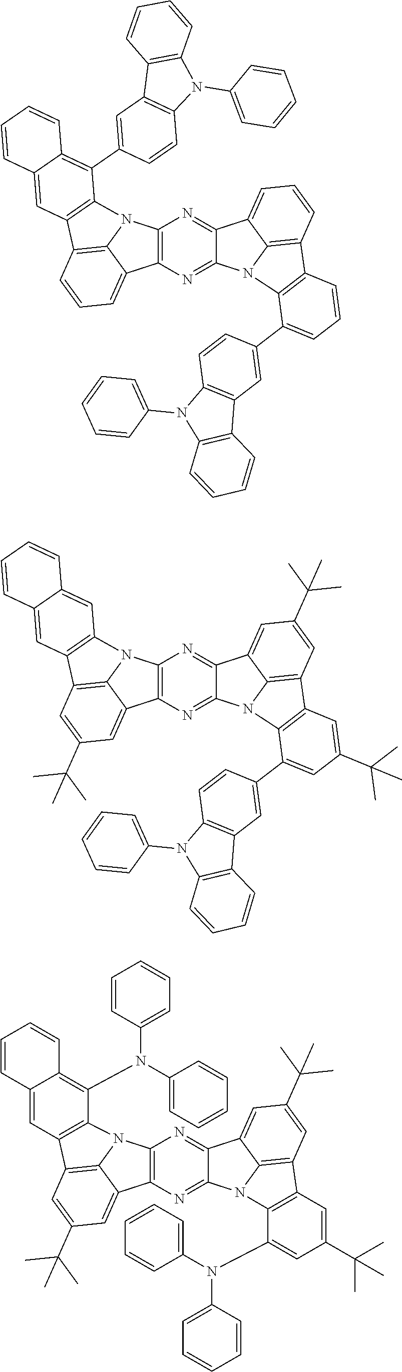 [SMILES: c1ccc(-n2c3ccccc3c3cc(-c4cccc5c6cccc7c8nc9c(nc8n(c45)c67)c4cccc5c6cc7ccccc7c(-c7ccc8c(c7)c7ccccc7n8-c7ccccc7)c6n9c54)ccc32)cc1 ; CC(C)(C)c1cc(-c2ccc3c(c2)c2ccccc2n3-c2ccccc2)c2c(c1)c1cc(C(C)(C)C)cc3c4nc5c(nc4n2c13)c1cc(C(C)(C)C)cc2c3cc4ccccc4cc3n5c21 ; CC(C)(C)c1cc(N(c2ccccc2)c2ccccc2)c2c(c1)c1cc(C(C)(C)C)cc3c4nc5c(nc4n2c13)c1cc(C(C)(C)C)cc2c3cc4ccccc4c(N(c4ccccc4)c4ccccc4)c3n5c21]

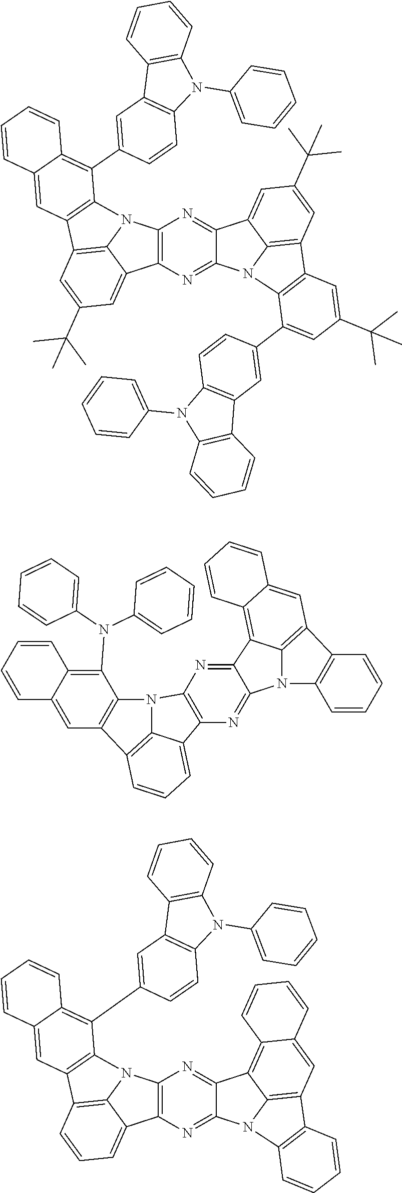 [SMILES: CC(C)(C)c1cc(-c2ccc3c(c2)c2ccccc2n3-c2ccccc2)c2c(c1)c1cc(C(C)(C)C)cc3c4nc5c(nc4n2c13)c1cc(C(C)(C)C)cc2c3cc4ccccc4c(-c4ccc6c(c4)c4ccccc4n6-c4ccccc4)c3n5c21 ; c1ccc(N(c2ccccc2)c2c3ccccc3cc3c4cccc5c6nc7c(nc6n(c23)c45)c2c3ccccc3cc3c4ccccc4n7c32)cc1 ; c1ccc(-n2c3ccccc3c3cc(-c4c5ccccc5cc5c6cccc7c8nc9c(nc8n(c45)c67)c4c5ccccc5cc5c6ccccc6n9c54)ccc32)cc1]

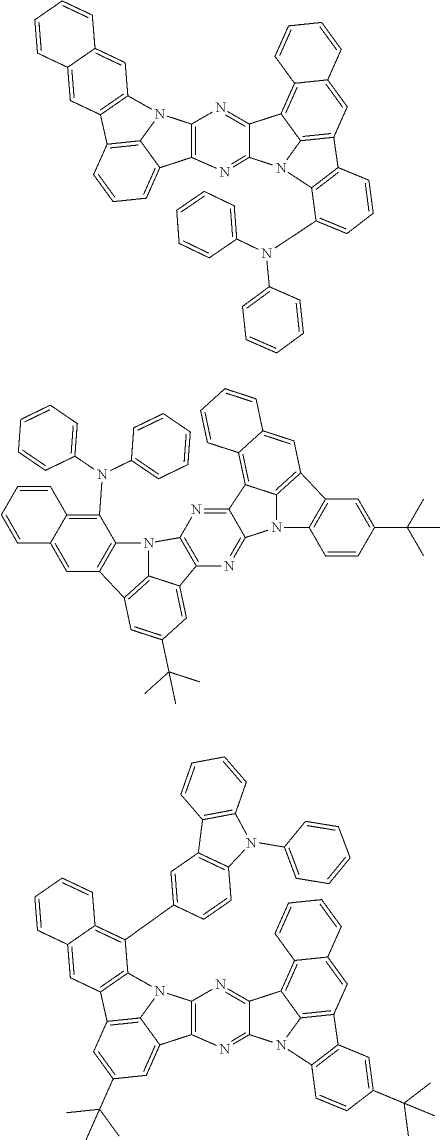 [SMILES: c1ccc(N(c2ccccc2)c2cccc3c4cc5ccccc5c5c6nc7c(nc6n(c23)c45)c2cccc3c4cc5ccccc5cc4n7c32)cc1 ; CC(C)(C)c1ccc2c(c1)c1cc3ccccc3c3c4nc5c(nc4n2c13)c1cc(C(C)(C)C)cc2c3cc4ccccc4c(N(c4ccccc4)c4ccccc4)c3n5c21 ; CC(C)(C)c1ccc2c(c1)c1cc3ccccc3c3c4nc5c(nc4n2c13)c1cc(C(C)(C)C)cc2c3cc4ccccc4c(-c4ccc6c(c4)c4ccccc4n6-c4ccccc4)c3n5c21]

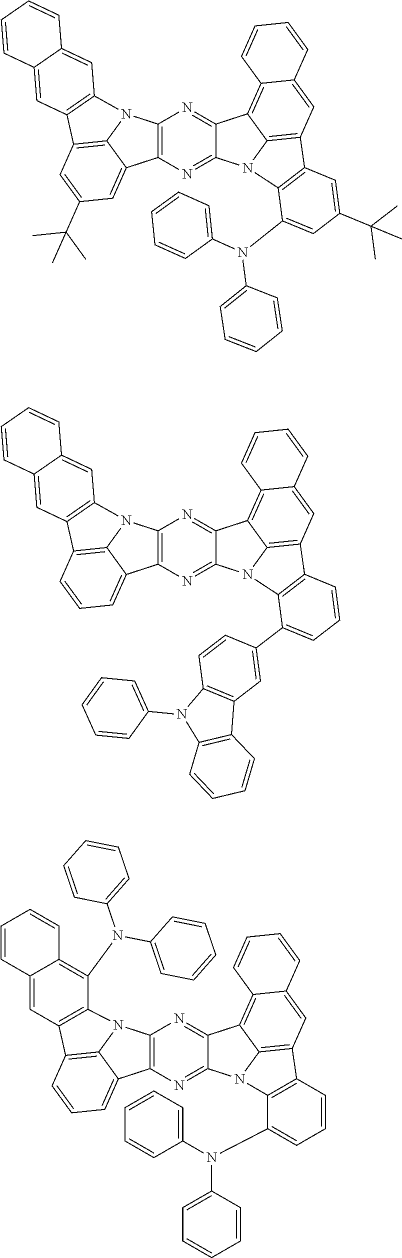 [SMILES: CC(C)(C)c1cc(N(c2ccccc2)c2ccccc2)c2c(c1)c1cc3ccccc3c3c4nc5c(nc4n2c13)c1cc(C(C)(C)C)cc2c3cc4ccccc4cc3n5c21 ; c1ccc(-n2c3ccccc3c3cc(-c4cccc5c6cc7ccccc7c7c8nc9c(nc8n(c45)c67)c4cccc5c6cc7ccccc7cc6n9c54)ccc32)cc1 ; c1ccc(N(c2ccccc2)c2c3ccccc3cc3c4cccc5c6nc7c(nc6n(c23)c45)c2c3ccccc3cc3c4cccc(N(c5ccccc5)c5ccccc5)c4n7c32)cc1]

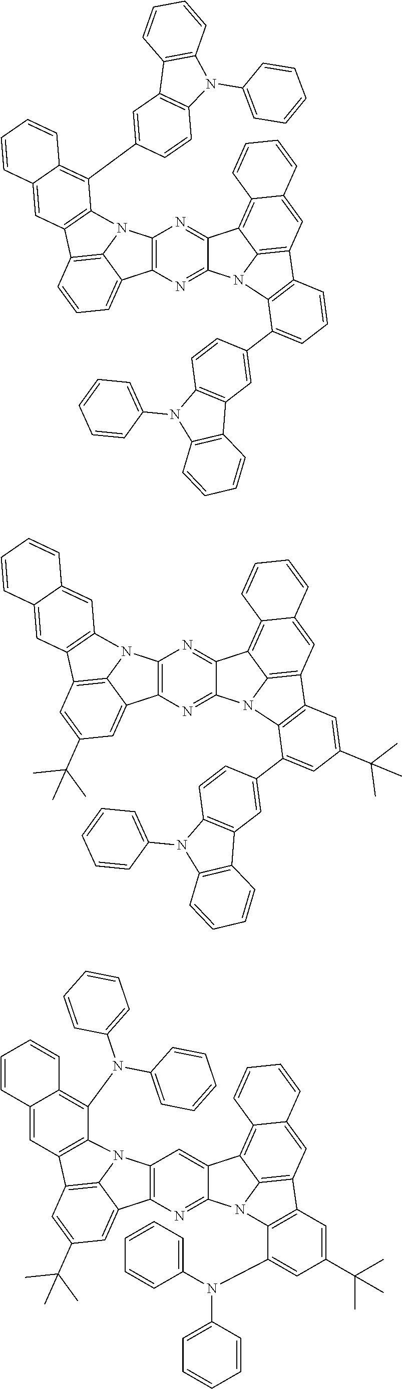 [SMILES: c1ccc(-n2c3ccccc3c3cc(-c4c5ccccc5cc5c6cccc7c8nc9c(nc8n(c45)c67)c4c5ccccc5cc5c6cccc(-c7ccc8c(c7)c7ccccc7n8-c7ccccc7)c6n9c54)ccc32)cc1 ; CC(C)(C)c1cc(-c2ccc3c(c2)c2ccccc2n3-c2ccccc2)c2c(c1)c1cc3ccccc3c3c4nc5c(nc4n2c13)c1cc(C(C)(C)C)cc2c3cc4ccccc4cc3n5c21 ; CC(C)(C)c1cc(N(c2ccccc2)c2ccccc2)c2c(c1)c1cc3ccccc3c3c4cc5c(nc4n2c13)c1cc(C(C)(C)C)cc2c3cc4ccccc4c(N(c4ccccc4)c4ccccc4)c3n5c21]

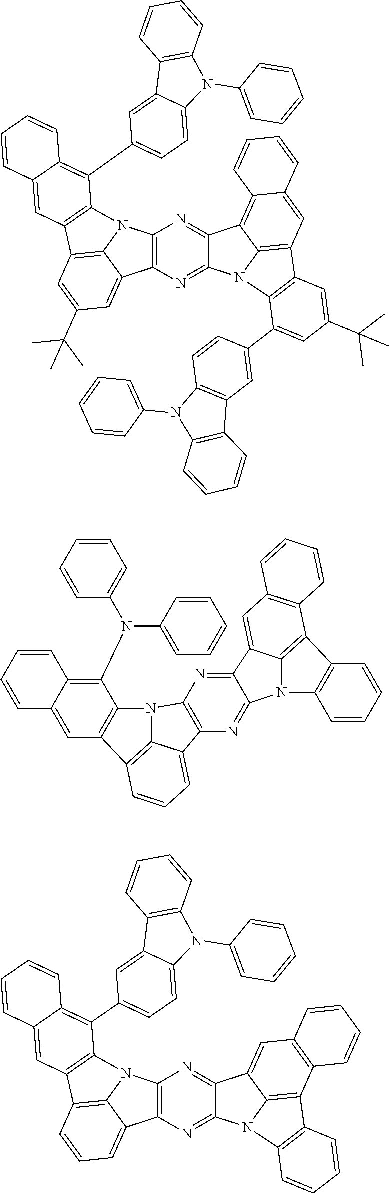 [SMILES: CC(C)(C)c1cc(-c2ccc3c(c2)c2ccccc2n3-c2ccccc2)c2c(c1)c1cc3ccccc3c3c4nc5c(nc4n2c13)c1cc(C(C)(C)C)cc2c3cc4ccccc4c(-c4ccc6c(c4)c4ccccc4n6-c4ccccc4)c3n5c21 ; c1ccc(N(c2ccccc2)c2c3ccccc3cc3c4cccc5c6nc7c(nc6n(c23)c45)c2cc3ccccc3c3c4ccccc4n7c23)cc1 ; c1ccc(-n2c3ccccc3c3cc(-c4c5ccccc5cc5c6cccc7c8nc9c(nc8n(c45)c67)c4cc5ccccc5c5c6ccccc6n9c45)ccc32)cc1]

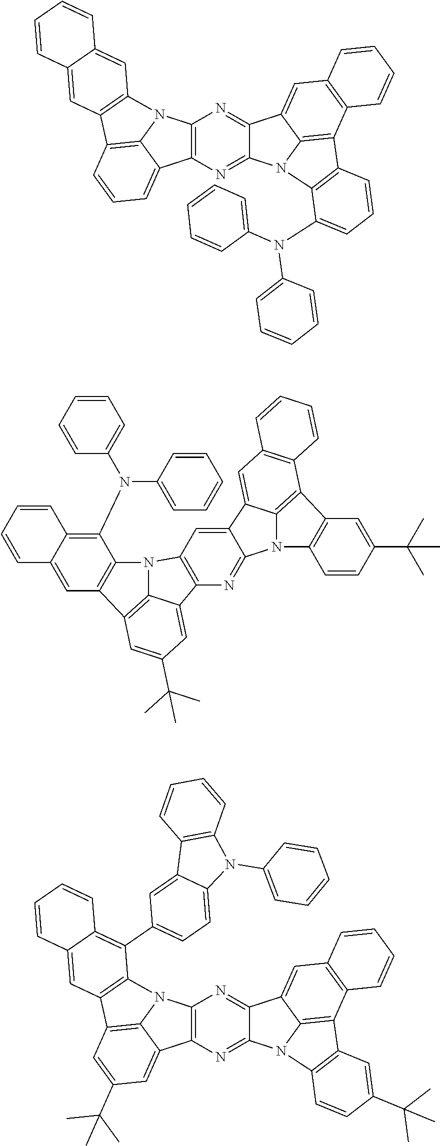 [SMILES: c1ccc(N(c2ccccc2)c2cccc3c4c5ccccc5cc5c6nc7c(nc6n(c23)c54)c2cccc3c4cc5ccccc5cc4n7c32)cc1 ; CC(C)(C)c1ccc2c(c1)c1c3ccccc3cc3c4cc5c(nc4n2c31)c1cc(C(C)(C)C)cc2c3cc4ccccc4c(N(c4ccccc4)c4ccccc4)c3n5c21 ; CC(C)(C)c1ccc2c(c1)c1c3ccccc3cc3c4nc5c(nc4n2c31)c1cc(C(C)(C)C)cc2c3cc4ccccc4c(-c4ccc6c(c4)c4ccccc4n6-c4ccccc4)c3n5c21]

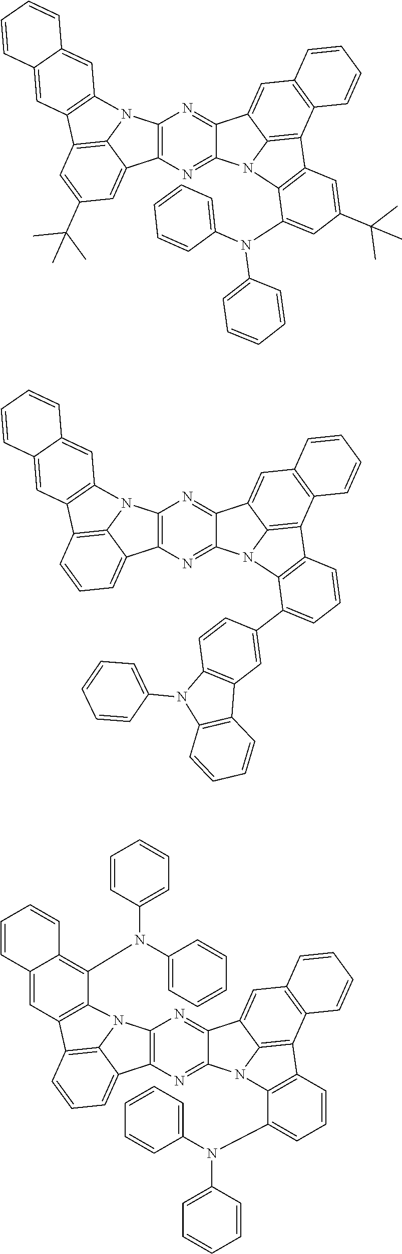 [SMILES: CC(C)(C)c1cc(N(c2ccccc2)c2ccccc2)c2c(c1)c1c3ccccc3cc3c4nc5c(nc4n2c31)c1cc(C(C)(C)C)cc2c3cc4ccccc4cc3n5c21 ; c1ccc(-n2c3ccccc3c3cc(-c4cccc5c6c7ccccc7cc7c8nc9c(nc8n(c45)c76)c4cccc5c6cc7ccccc7cc6n9c54)ccc32)cc1 ; c1ccc(N(c2ccccc2)c2c3ccccc3cc3c4cccc5c6nc7c(nc6n(c23)c45)c2cc3ccccc3c3c4cccc(N(c5ccccc5)c5ccccc5)c4n7c23)cc1]

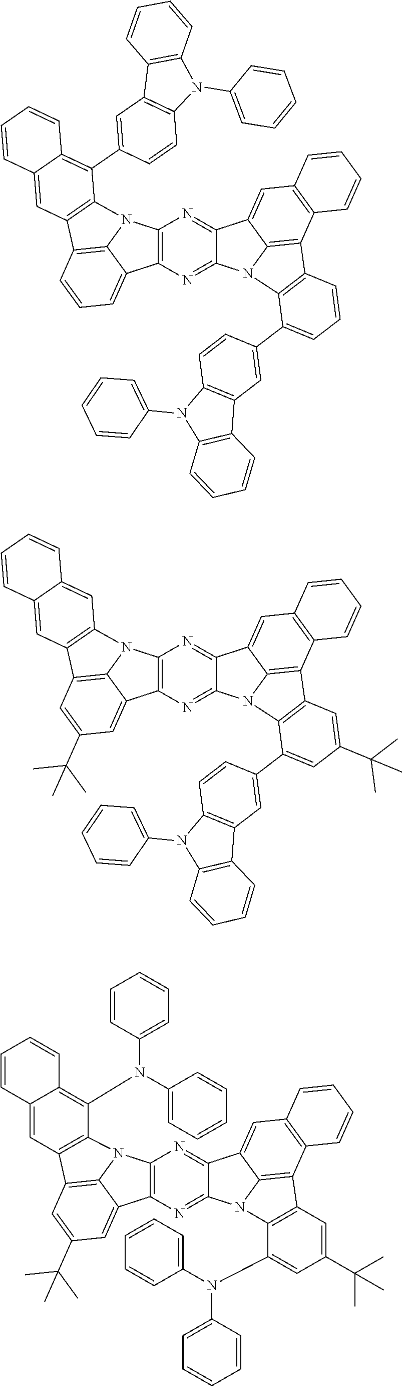 [SMILES: c1ccc(-n2c3ccccc3c3cc(-c4c5ccccc5cc5c6cccc7c8nc9c(nc8n(c45)c67)c4cc5ccccc5c5c6cccc(-c7ccc8c(c7)c7ccccc7n8-c7ccccc7)c6n9c45)ccc32)cc1 ; CC(C)(C)c1cc(-c2ccc3c(c2)c2ccccc2n3-c2ccccc2)c2c(c1)c1c3ccccc3cc3c4nc5c(nc4n2c31)c1cc(C(C)(C)C)cc2c3cc4ccccc4cc3n5c21 ; CC(C)(C)c1cc(N(c2ccccc2)c2ccccc2)c2c(c1)c1c3ccccc3cc3c4nc5c(nc4n2c31)c1cc(C(C)(C)C)cc2c3cc4ccccc4c(N(c4ccccc4)c4ccccc4)c3n5c21]

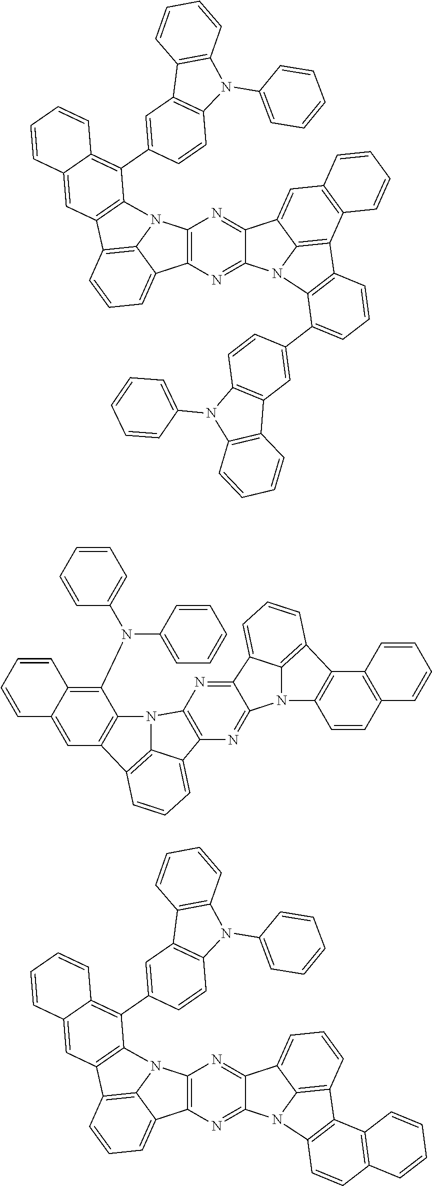

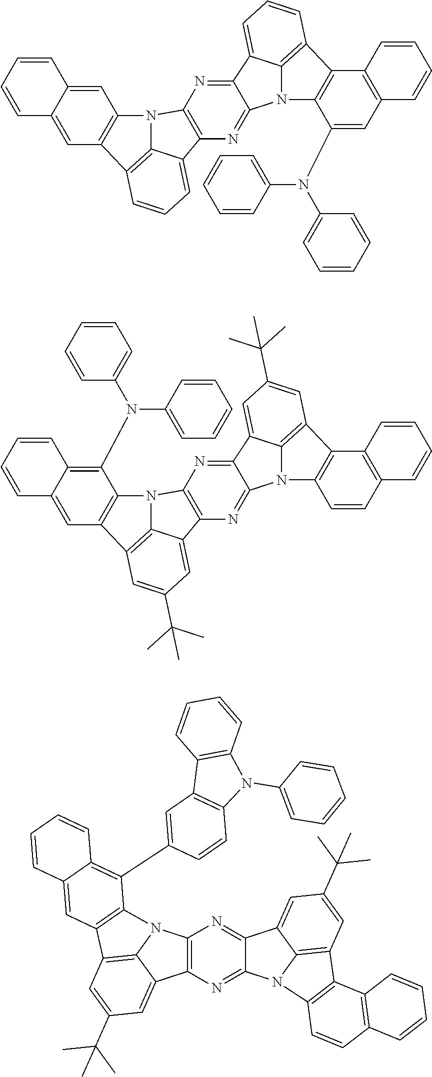

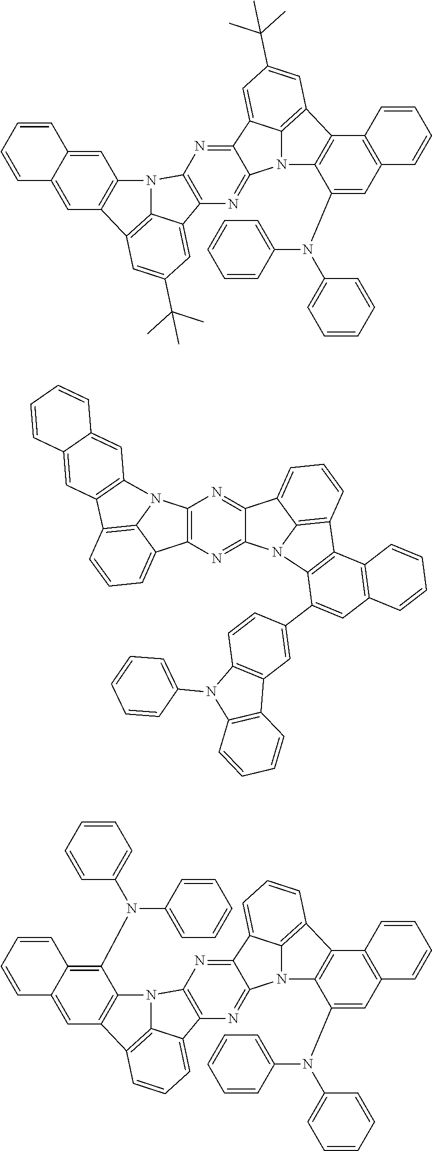 [SMILES: CC(C)(C)c1cc2c3cc4ccccc4cc3n3c4nc5c6cc(C(C)(C)C)cc7c8c9ccccc9cc(N(c9ccccc9)c9ccccc9)c8n(c5nc4c(c1)c23)c67 ; c1ccc(-n2c3ccccc3c3cc(-c4cc5ccccc5c5c6cccc7c8nc9c(nc8n(c45)c76)c4cccc5c6cc7ccccc7cc6n9c54)ccc32)cc1 ; c1ccc(N(c2ccccc2)c2c3ccccc3cc3c4cccc5c6nc7c(nc6n(c23)c45)c2cccc3c4c5ccccc5cc(N(c5ccccc5)c5ccccc5)c4n7c23)cc1]

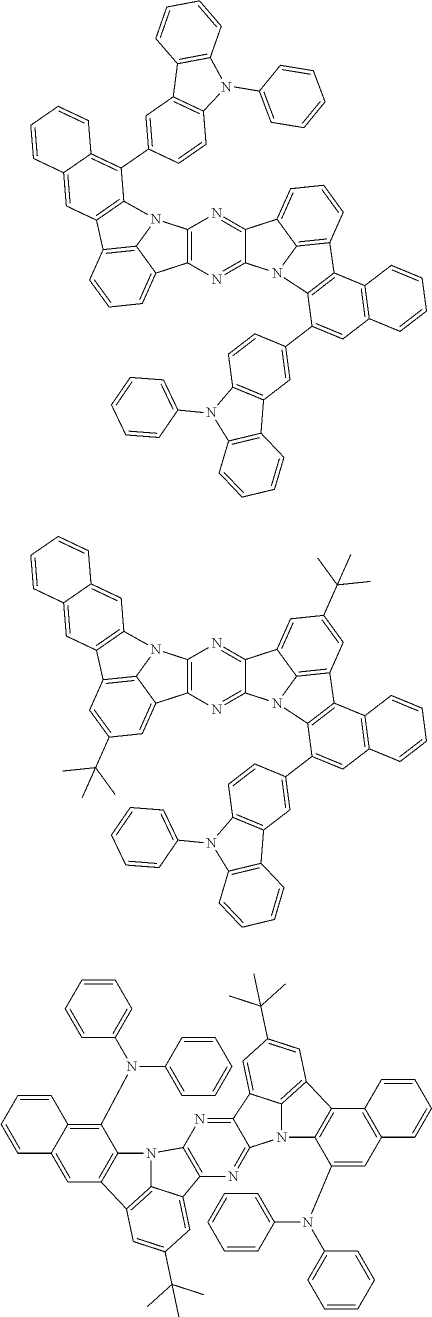 [SMILES: c1ccc(-n2c3ccccc3c3cc(-c4c5ccccc5cc5c6cccc7c8nc9c(nc8n(c45)c67)c4cccc5c6c7ccccc7cc(-c7ccc8c(c7)c7ccccc7n8-c7ccccc7)c6n9c45)ccc32)cc1 ; CC(C)(C)c1cc2c3cc4ccccc4cc3n3c4nc5c6cc(C(C)(C)C)cc7c8c9ccccc9cc(-c9ccc%10c(c9)c9ccccc9n%10-c9ccccc9)c8n(c5nc4c(c1)c23)c67 ; CC(C)(C)c1cc2c3cc4ccccc4c(N(c4ccccc4)c4ccccc4)c3n3c4nc5c6cc(C(C)(C)C)cc7c8c9ccccc9cc(N(c9ccccc9)c9ccccc9)c8n(c5nc4c(c1)c23)c67]

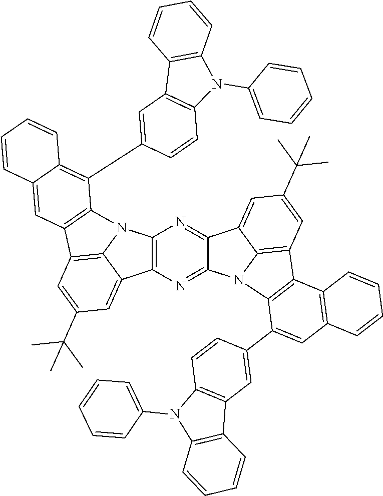 [SMILES: CC(C)(C)c1cc2c3cc4ccccc4c(-c4ccc5c(c4)c4ccccc4n5-c4ccccc4)c3n3c4nc5c6cc(C(C)(C)C)cc7c8c9ccccc9cc(-c9ccc%10c(c9)c9ccccc9n%10-c9ccccc9)c8n(c5nc4c(c1)c23)c67]

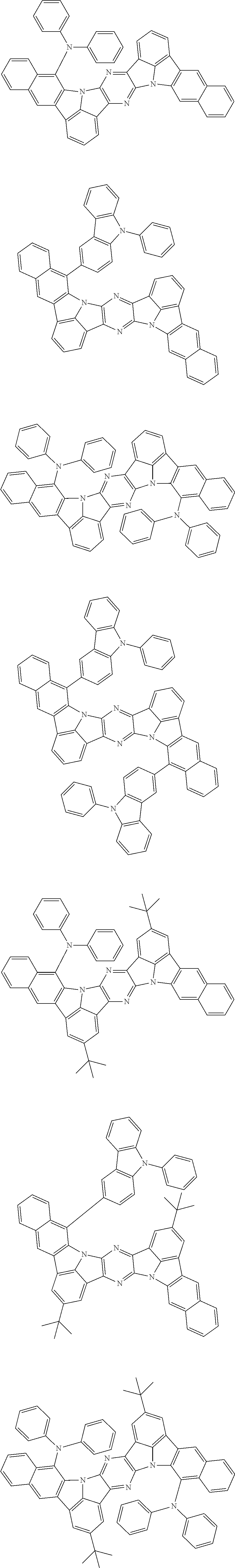

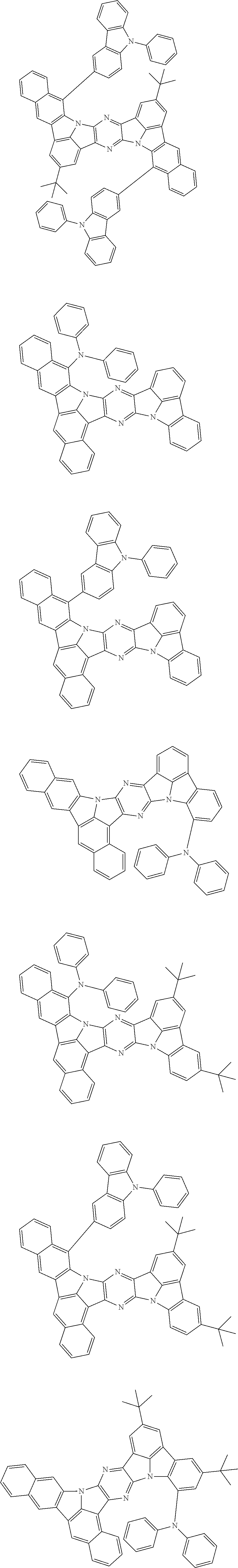

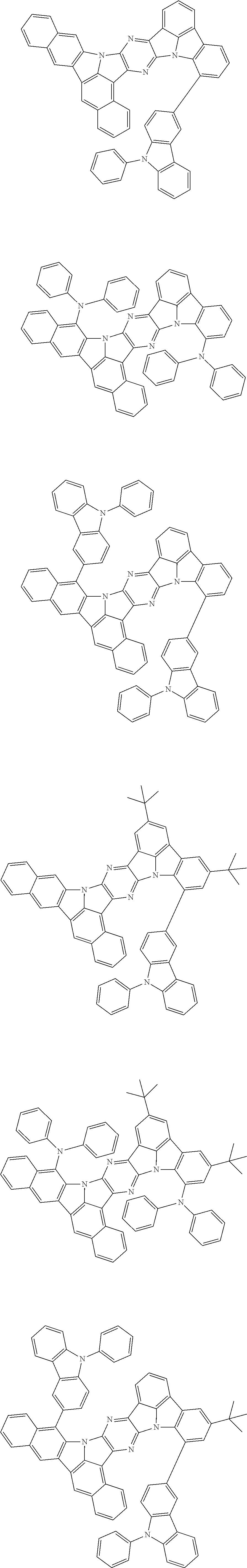

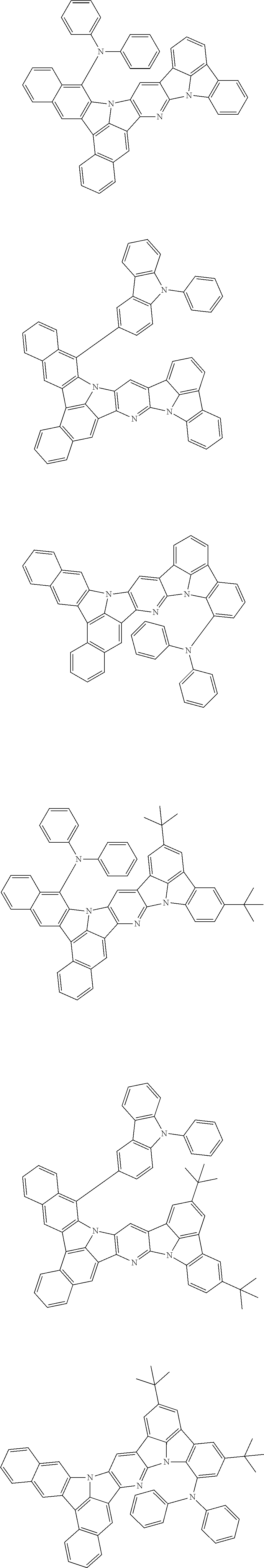

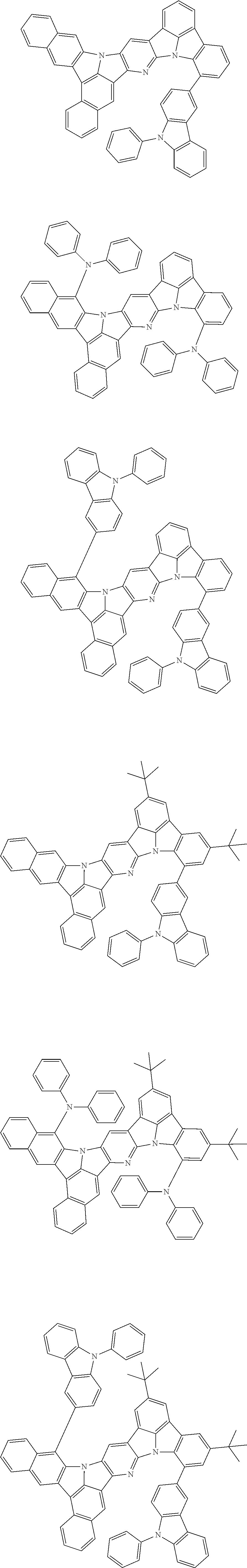

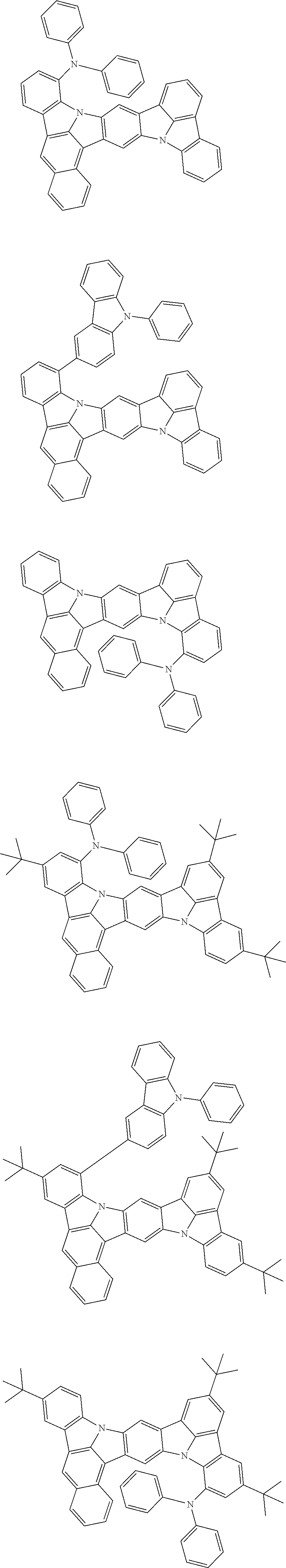

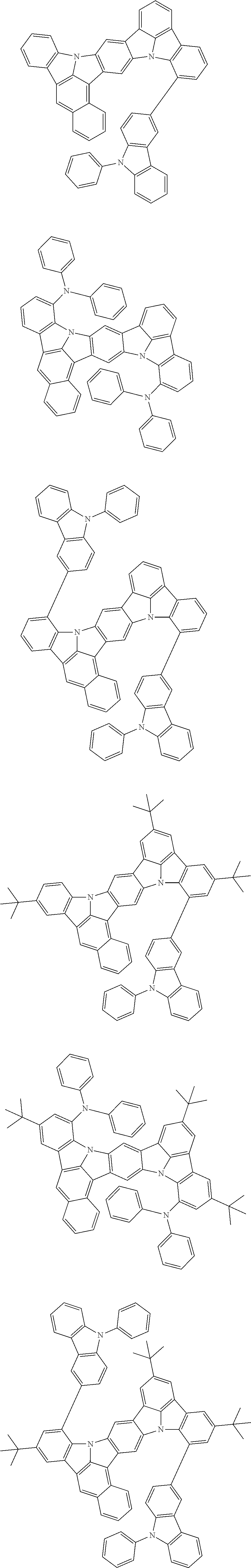

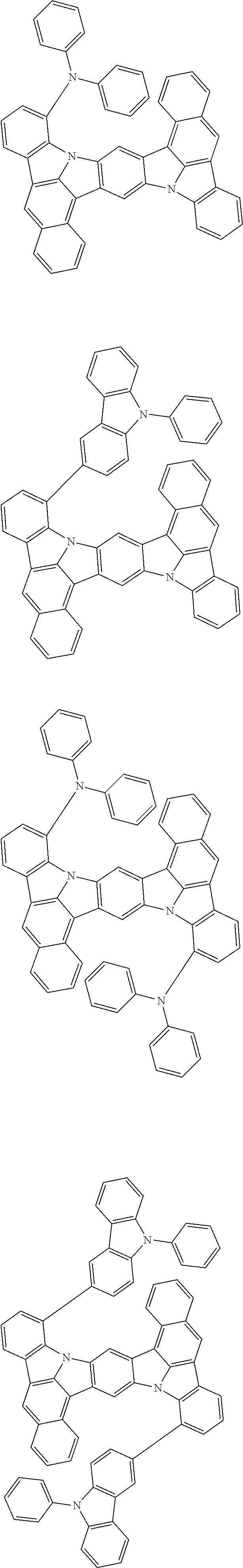 [SMILES: c1ccc(N(c2ccccc2)c2cccc3c4cc5ccccc5c5c6cc7c(cc6n(c23)c45)c2c3ccccc3cc3c4ccccc4n7c32)cc1 ; c1ccc(-n2c3ccccc3c3cc(-c4cccc5c6cc7ccccc7c7c8cc9c(cc8n(c45)c67)c4c5ccccc5cc5c6ccccc6n9c54)ccc32)cc1 ; c1ccc(N(c2ccccc2)c2cccc3c4cc5ccccc5c5c6cc7c(cc6n(c23)c45)c2c3ccccc3cc3c4cccc(N(c5ccccc5)c5ccccc5)c4n7c32)cc1 ; c1ccc(-n2c3ccccc3c3cc(-c4cccc5c6cc7ccccc7c7c8cc9c(cc8n(c45)c67)c4c5ccccc5cc5c6cccc(-c7ccc8c(c7)c7ccccc7n8-c7ccccc7)c6n9c54)ccc32)cc1]

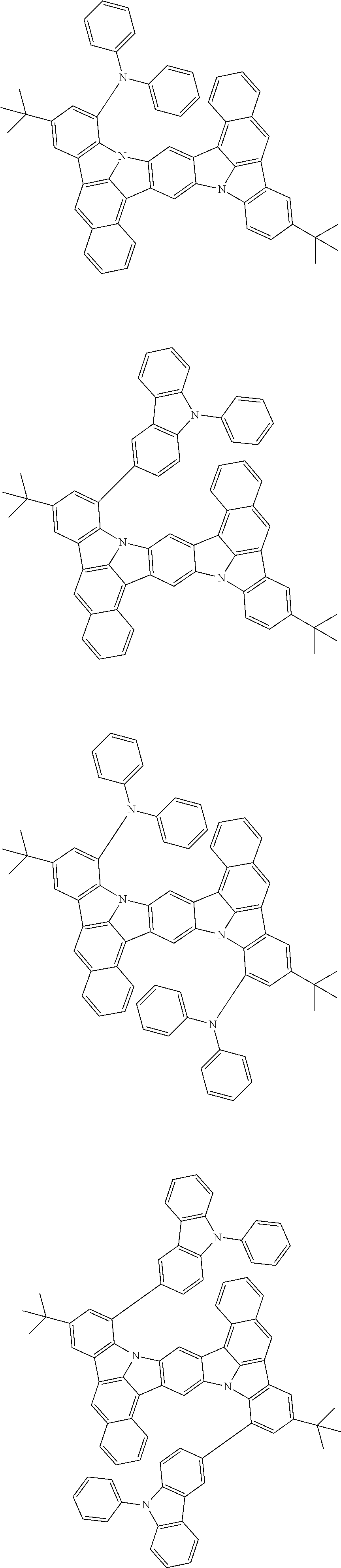

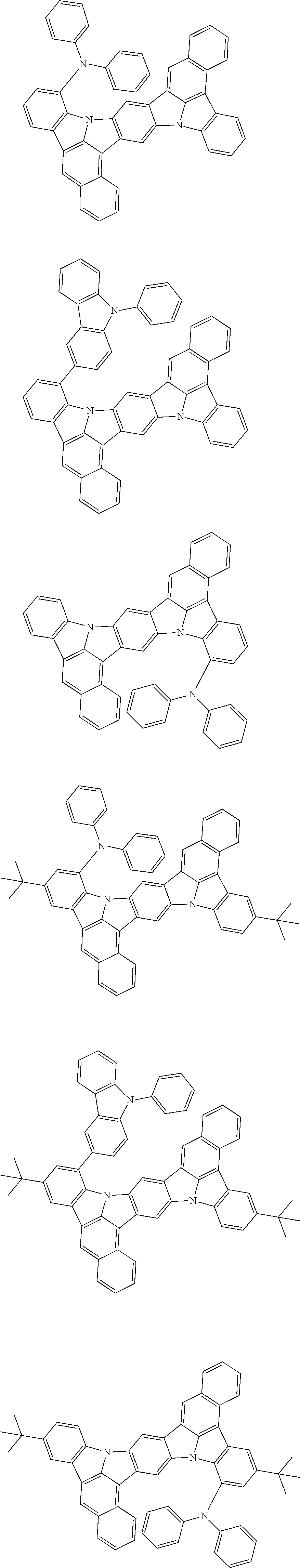

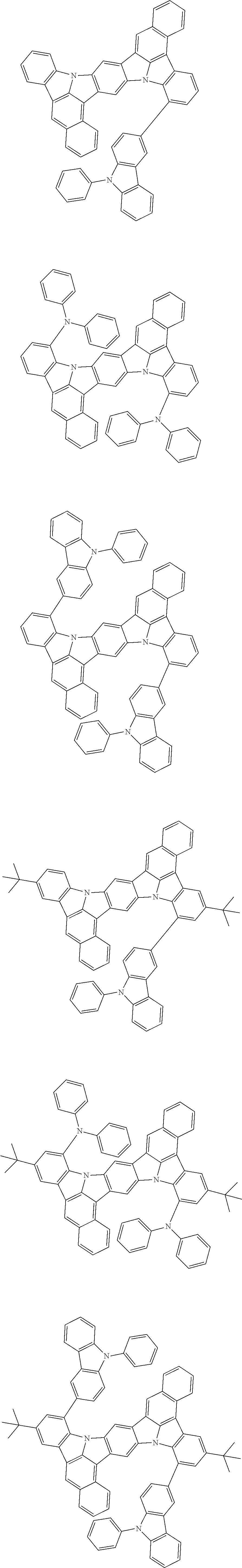

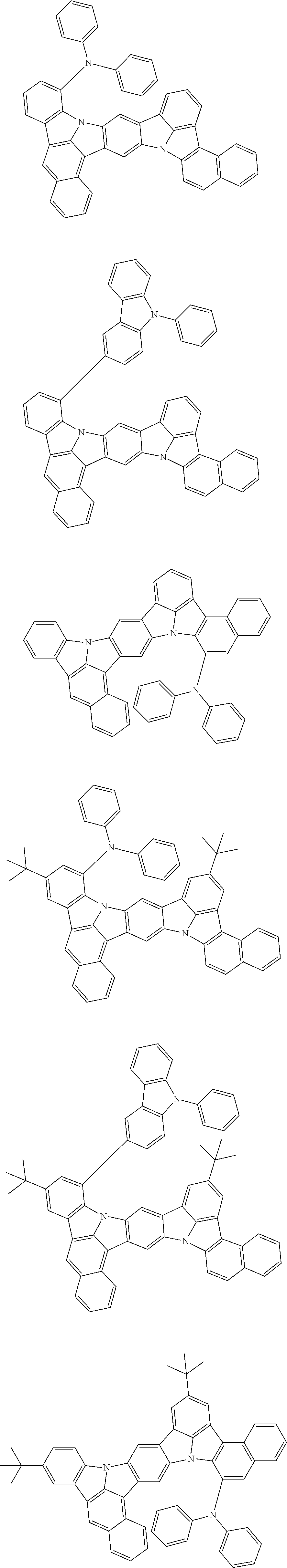

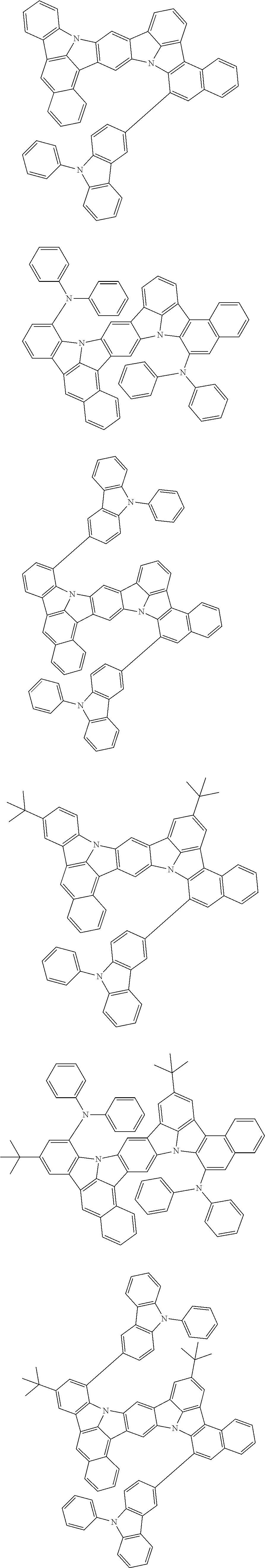

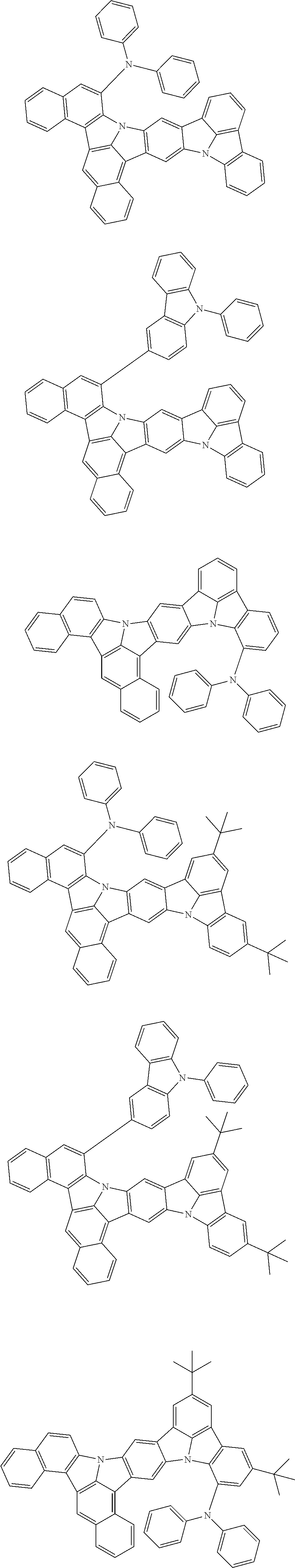

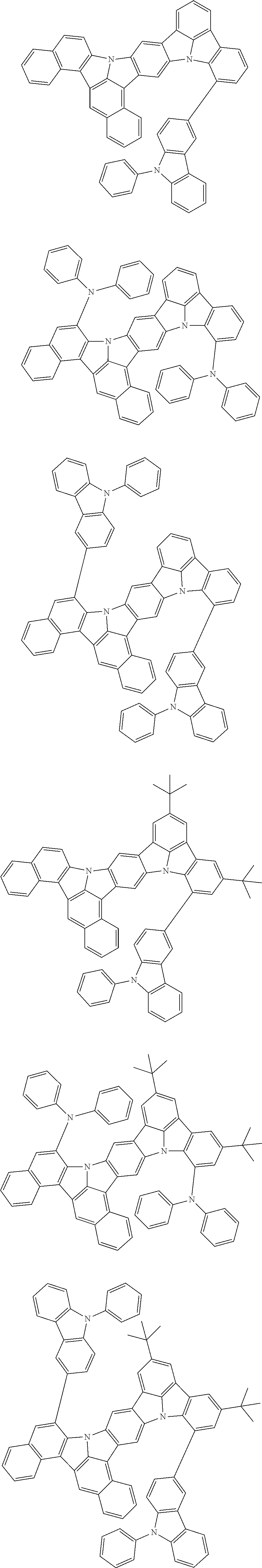

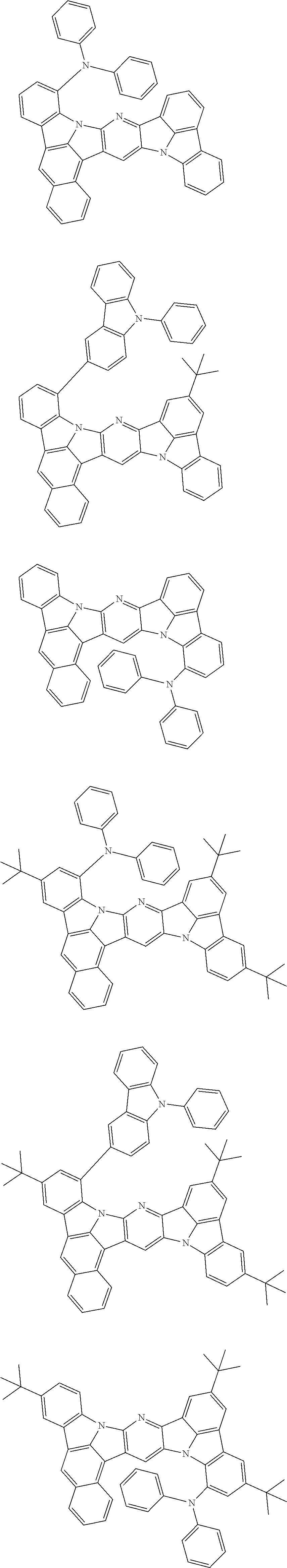

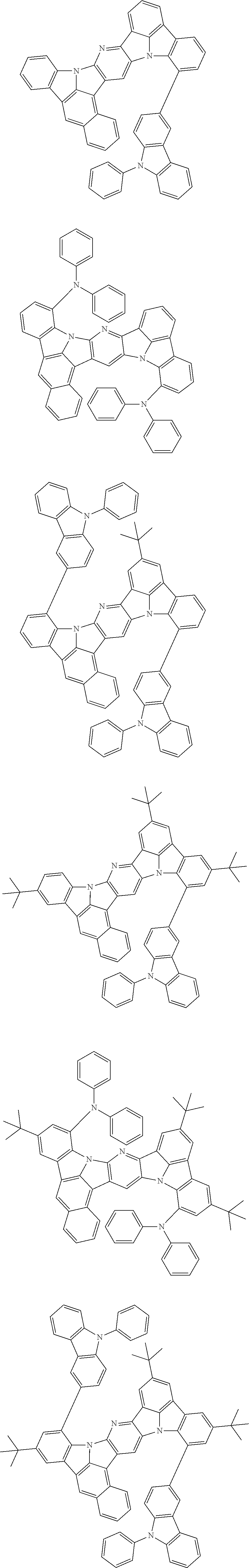

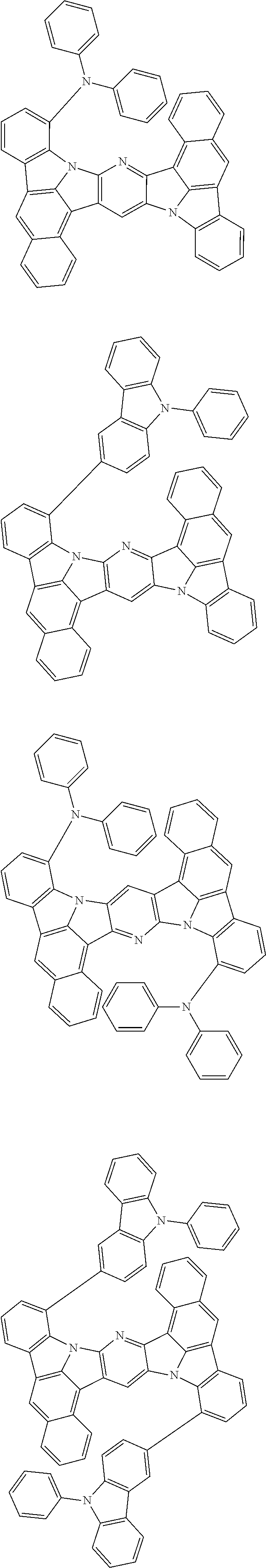 [SMILES: c1ccc(N(c2ccccc2)c2cccc3c4cc5ccccc5c5c6cc7c(nc6n(c23)c45)c2c3ccccc3cc3c4ccccc4n7c32)cc1 ; c1ccc(-n2c3ccccc3c3cc(-c4cccc5c6cc7ccccc7c7c8cc9c(nc8n(c45)c67)c4c5ccccc5cc5c6ccccc6n9c54)ccc32)cc1 ; c1ccc(N(c2ccccc2)c2cccc3c4cc5ccccc5c5c6nc7c(cc6n(c23)c45)c2c3ccccc3cc3c4cccc(N(c5ccccc5)c5ccccc5)c4n7c32)cc1 ; c1ccc(-n2c3ccccc3c3cc(-c4cccc5c6cc7ccccc7c7c8nc9c(cc8n(c45)c67)c4c5ccccc5cc5c6cccc(-c7ccc8c(c7)c7ccccc7n8-c7ccccc7)c6n9c54)ccc32)cc1]

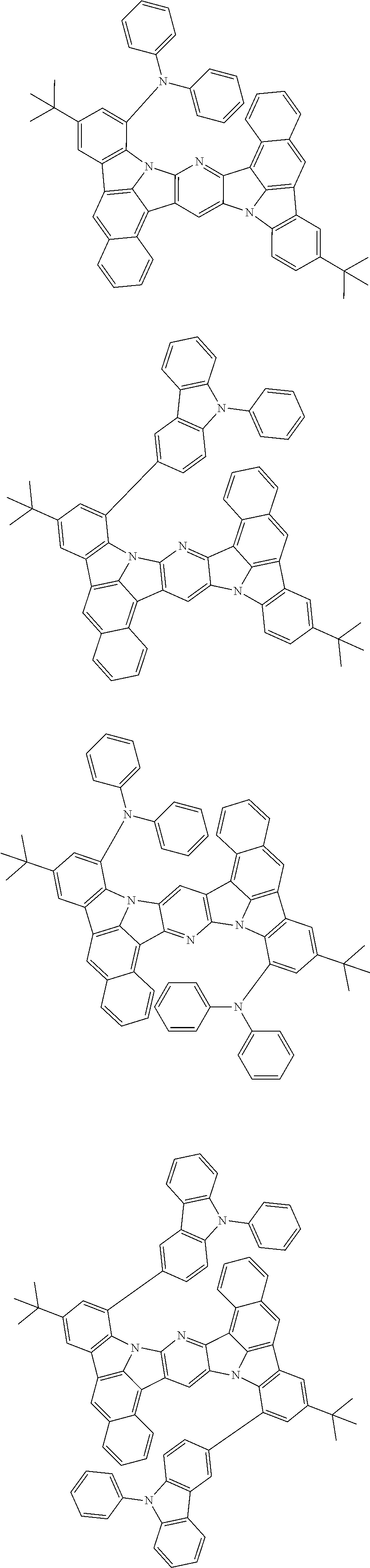

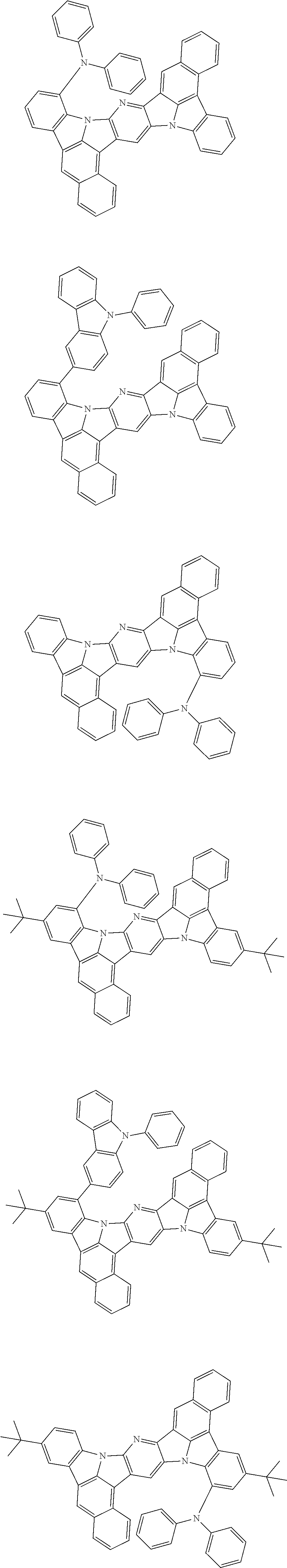

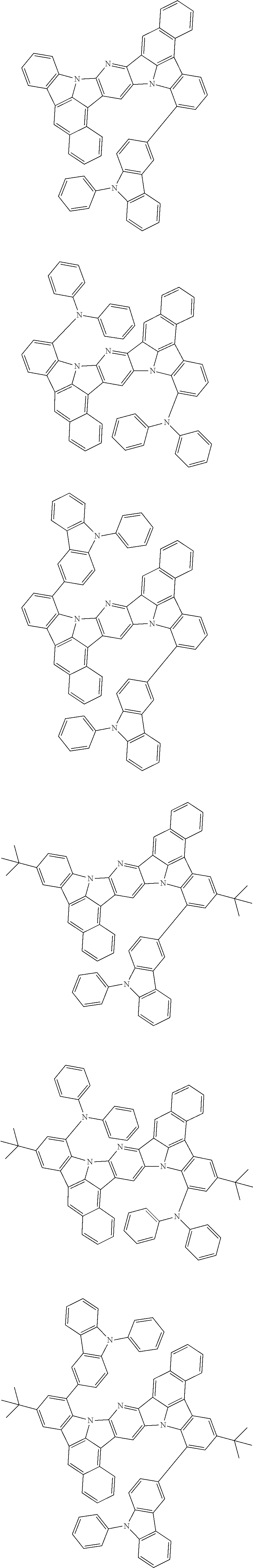

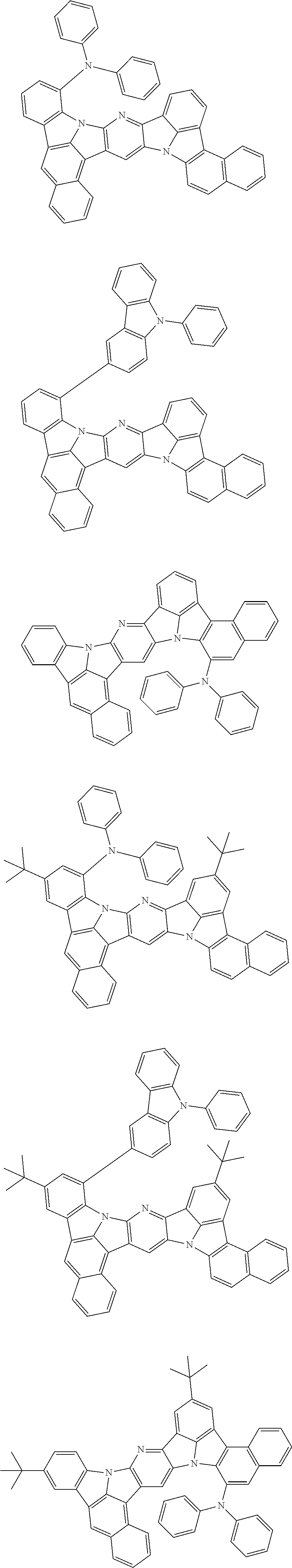

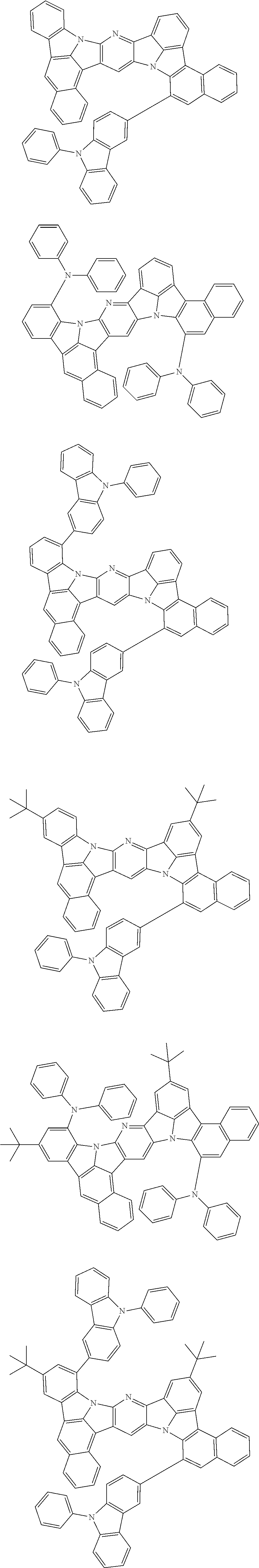

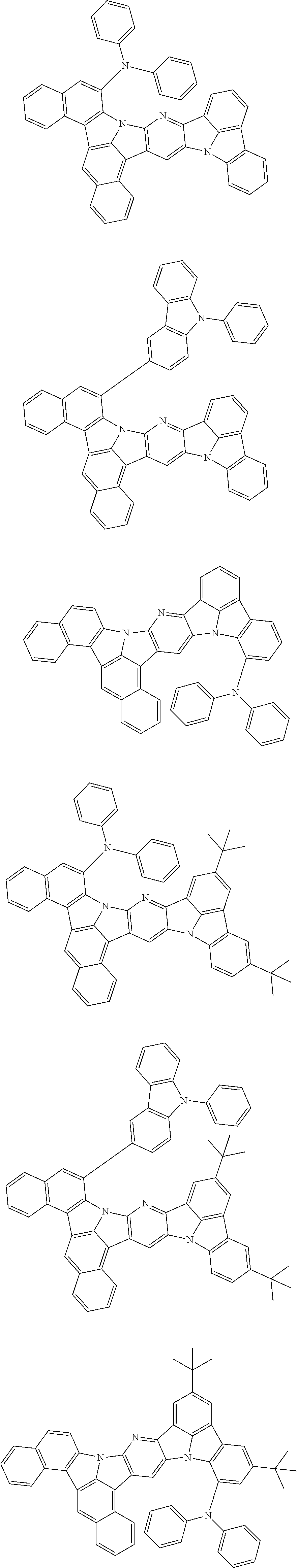

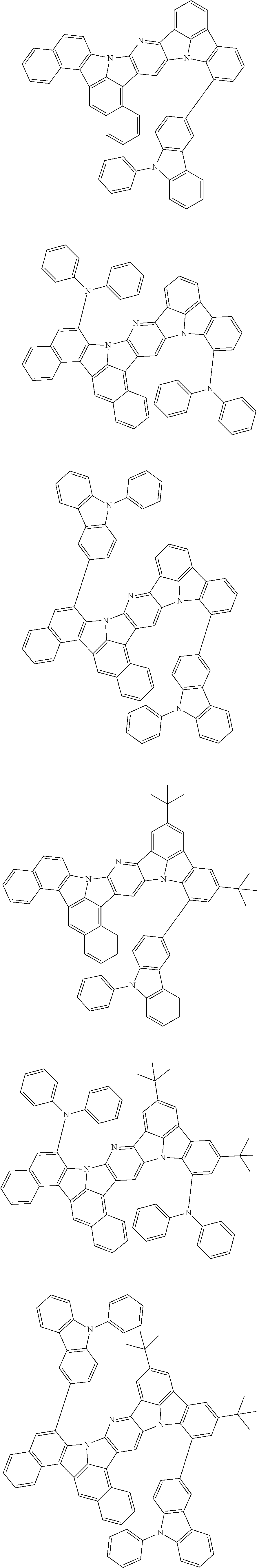

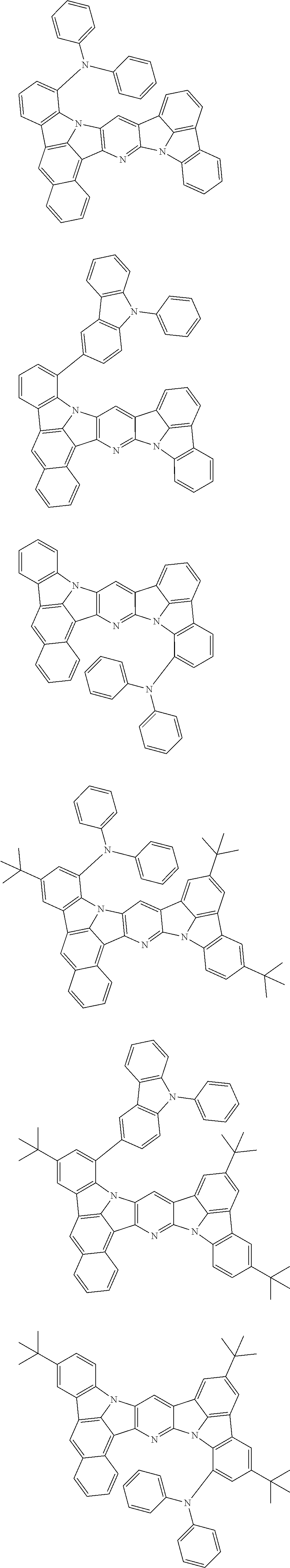

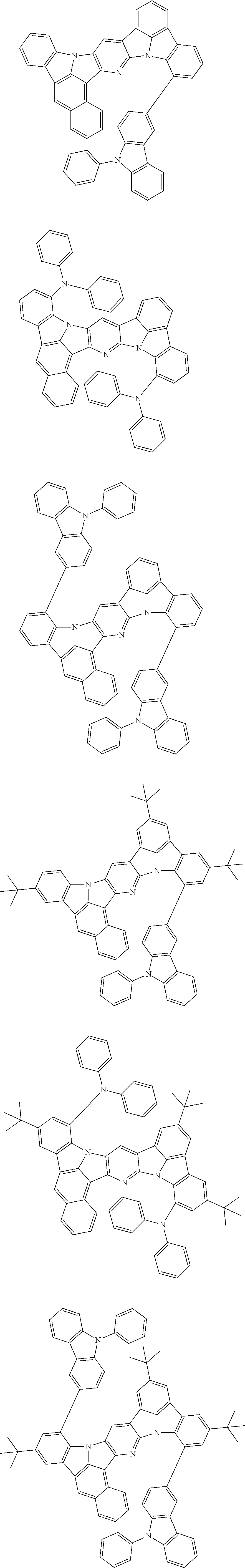

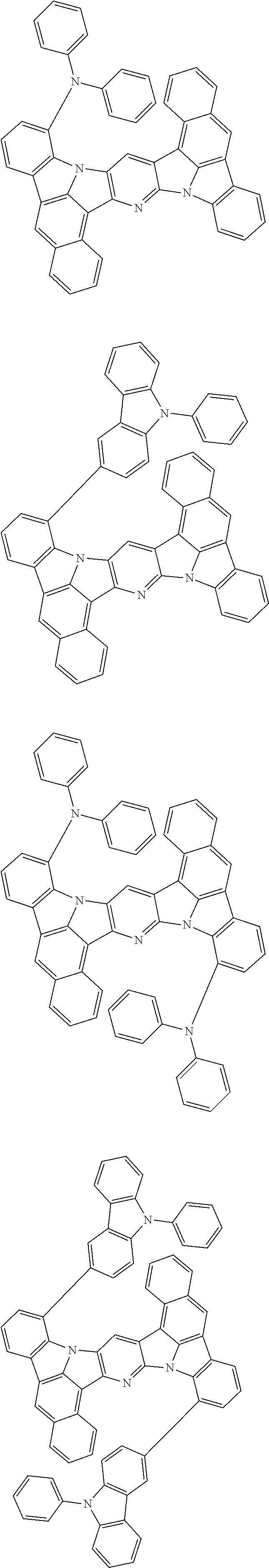 [SMILES: c1ccc(N(c2ccccc2)c2cccc3c4cc5ccccc5c5c6nc7c(cc6n(c23)c45)c2c3ccccc3cc3c4ccccc4n7c32)cc1 ; c1ccc(-n2c3ccccc3c3cc(-c4cccc5c6cc7ccccc7c7c8nc9c(cc8n(c45)c67)c4c5ccccc5cc5c6ccccc6n9c54)ccc32)cc1 ; c1ccc(N(c2ccccc2)c2cccc3c4cc5ccccc5c5c6nc7c(cc6n(c23)c45)c2c3ccccc3cc3c4cccc(N(c5ccccc5)c5ccccc5)c4n7c32)cc1 ; c1ccc(-n2c3ccccc3c3cc(-c4cccc5c6cc7ccccc7c7c8nc9c(cc8n(c45)c67)c4c5ccccc5cc5c6cccc(-c7ccc8c(c7)c7ccccc7n8-c7ccccc7)c6n9c54)ccc32)cc1]

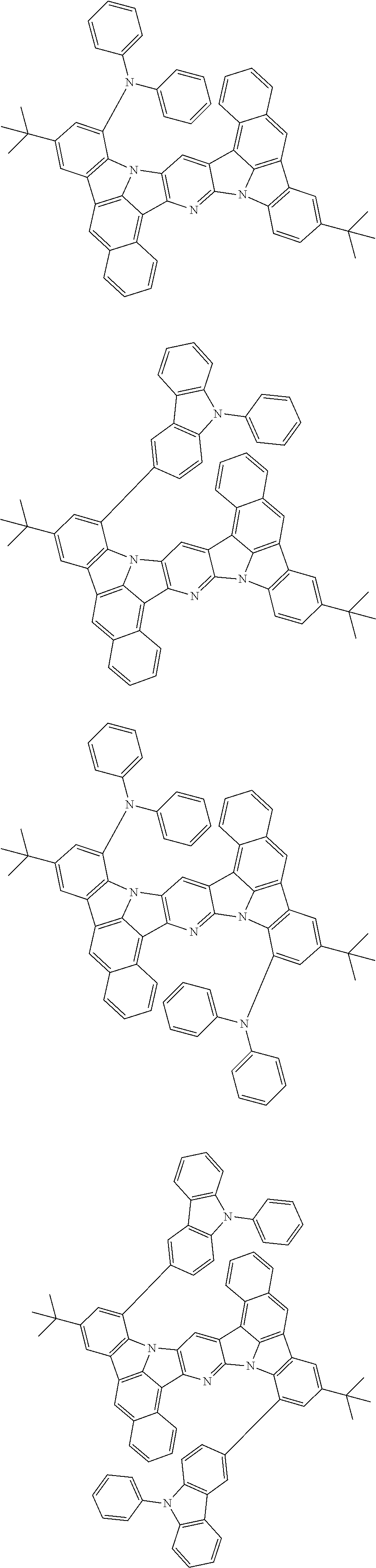

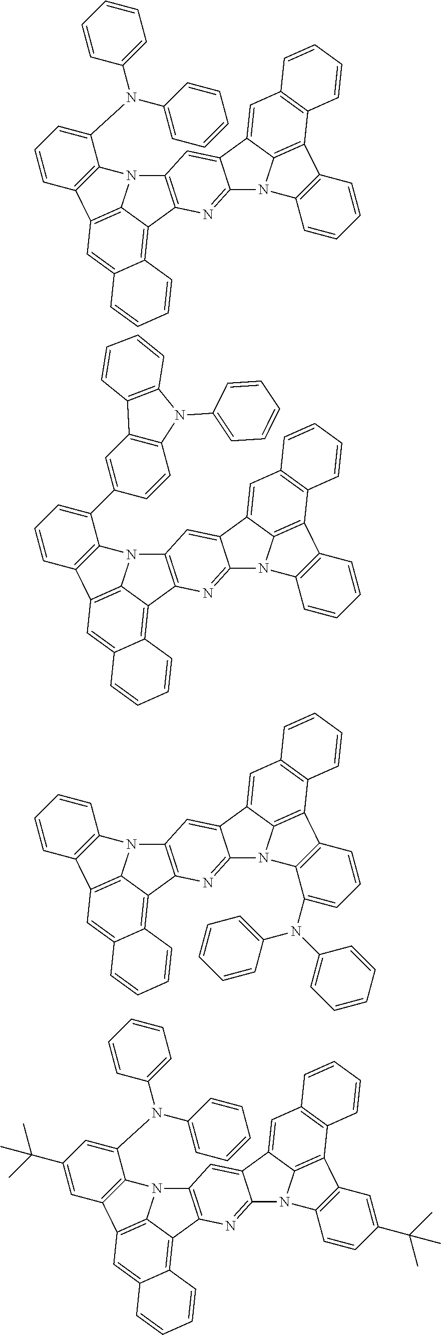

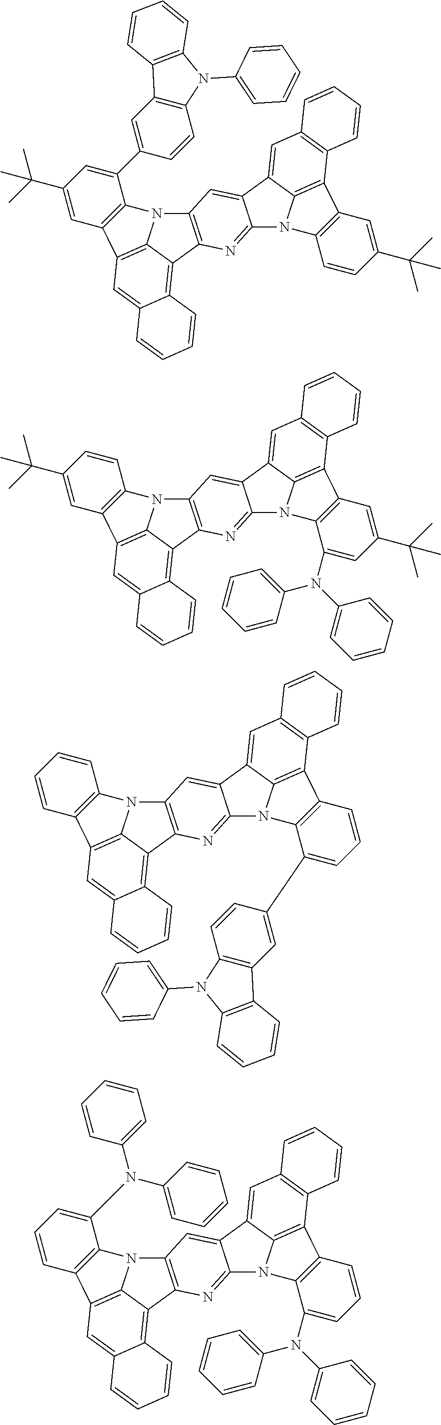

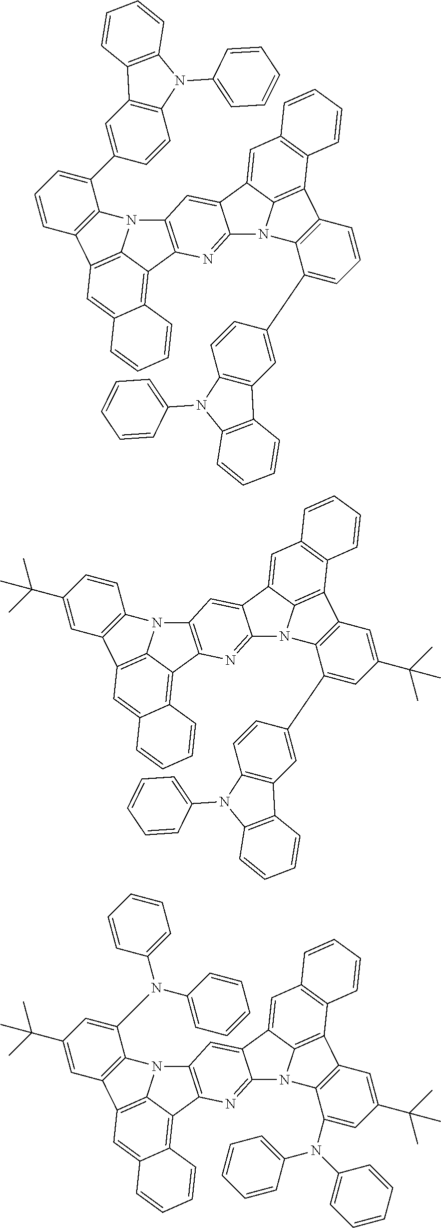

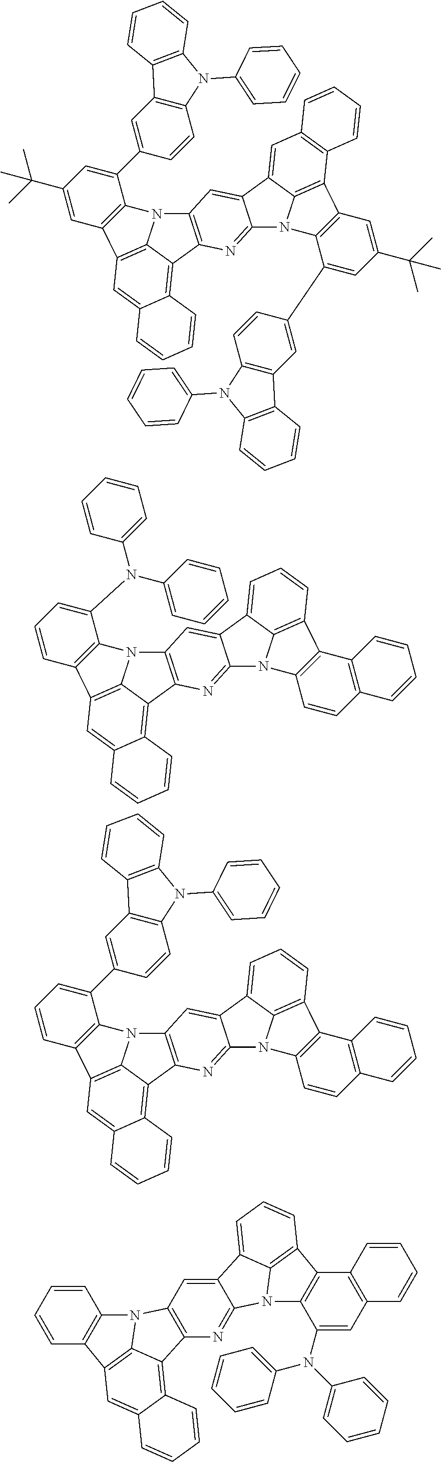

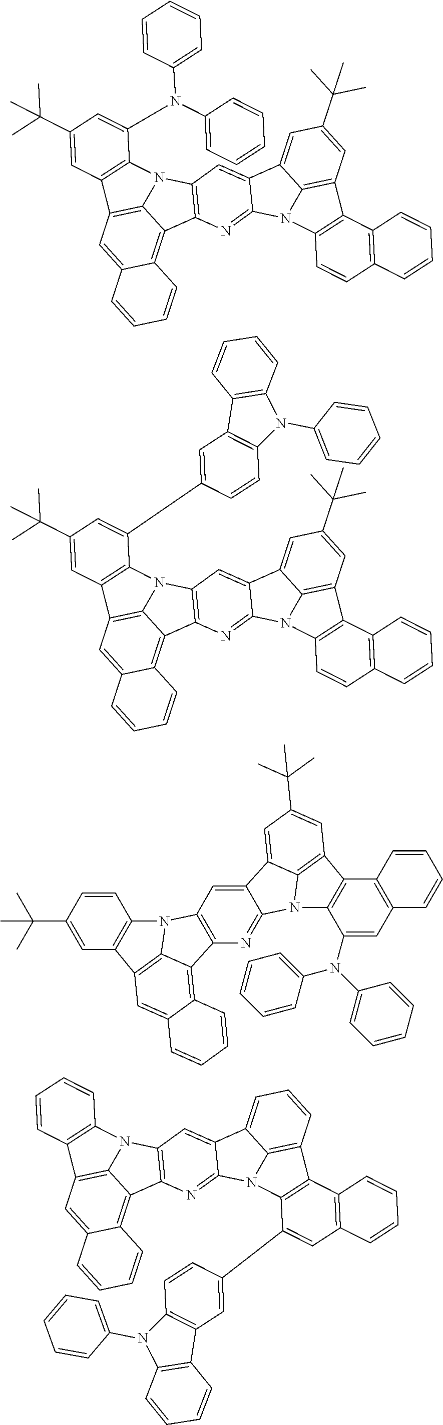 [SMILES: CC(C)(C)c1cc(N(c2ccccc2)c2ccccc2)c2c(c1)c1cc3ccccc3c3c4nc5c(cc4n2c13)c1cc(C(C)(C)C)cc2c3c4ccccc4ccc3n5c12 ; CC(C)(C)c1cc(-c2ccc3c(c2)c2ccccc2n3-c2ccccc2)c2c(c1)c1cc3ccccc3c3c4nc5c(cc4n2c13)c1cc(C(C)(C)C)cc2c3c4ccccc4ccc3n5c12 ; CC(C)(C)c1ccc2c(c1)c1cc3ccccc3c3c4nc5c(cc4n2c13)c1cc(C(C)(C)C)cc2c3c4ccccc4cc(N(c4ccccc4)c4ccccc4)c3n5c12 ; c1ccc(-n2c3ccccc3c3cc(-c4cc5ccccc5c5c6cccc7c8cc9c(nc8n(c45)c76)c4c5ccccc5cc5c6ccccc6n9c54)ccc32)cc1]

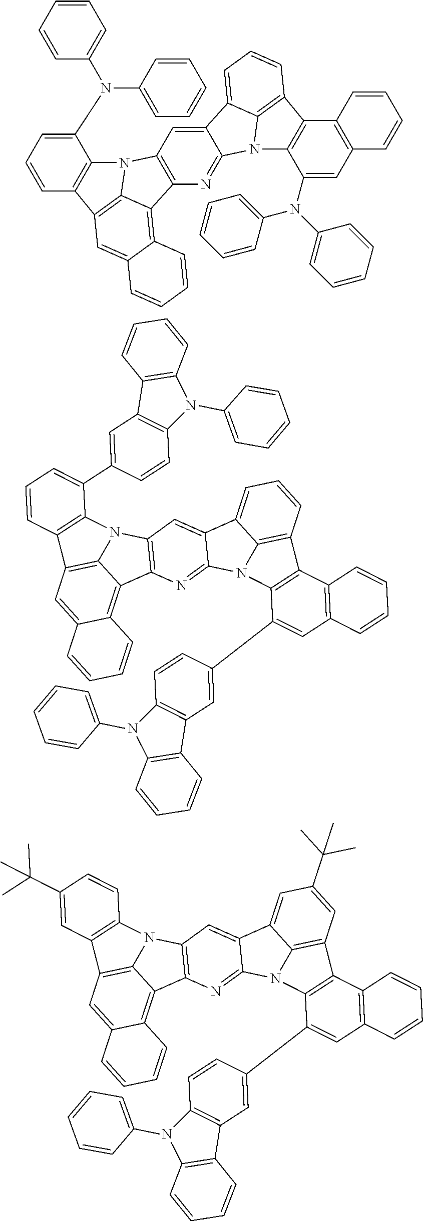

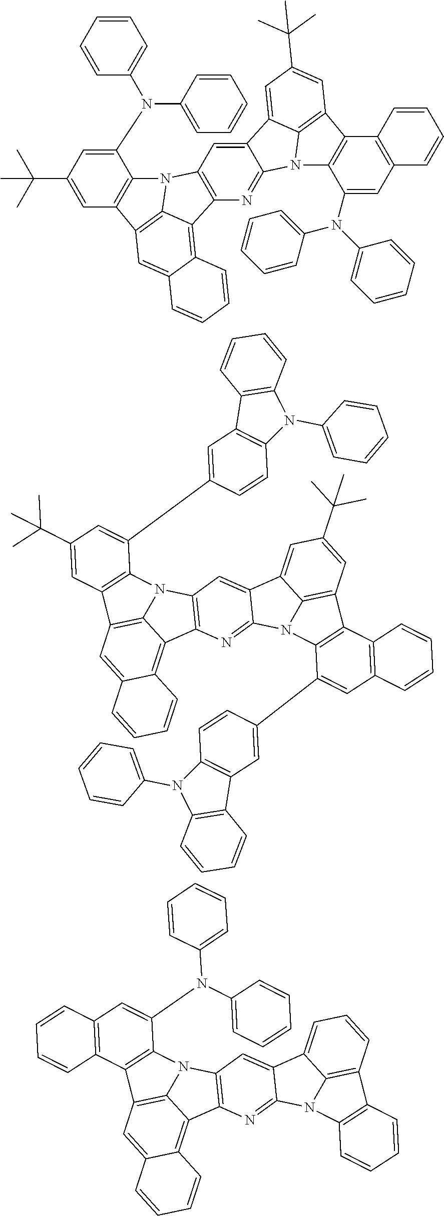

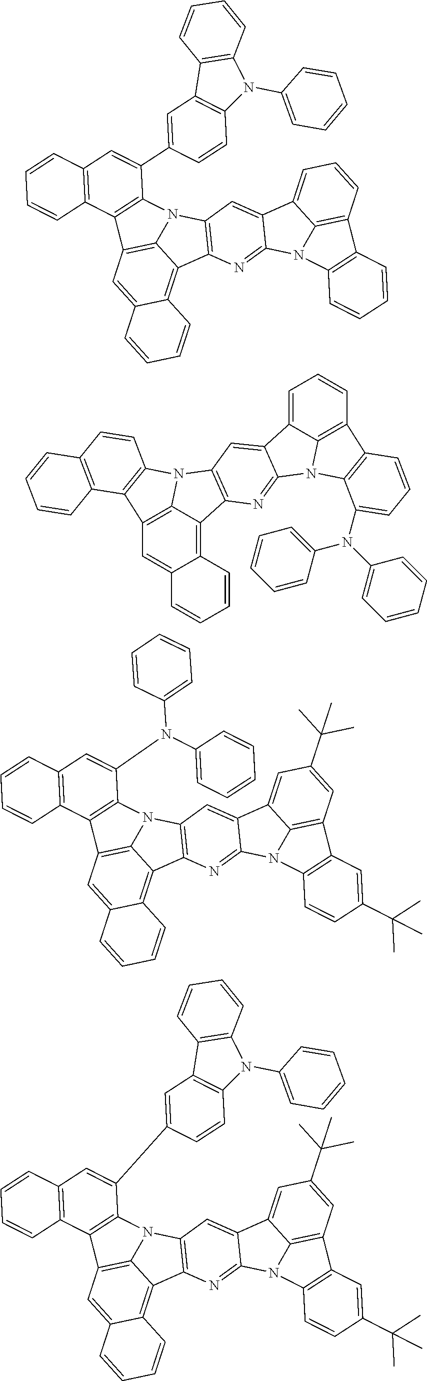

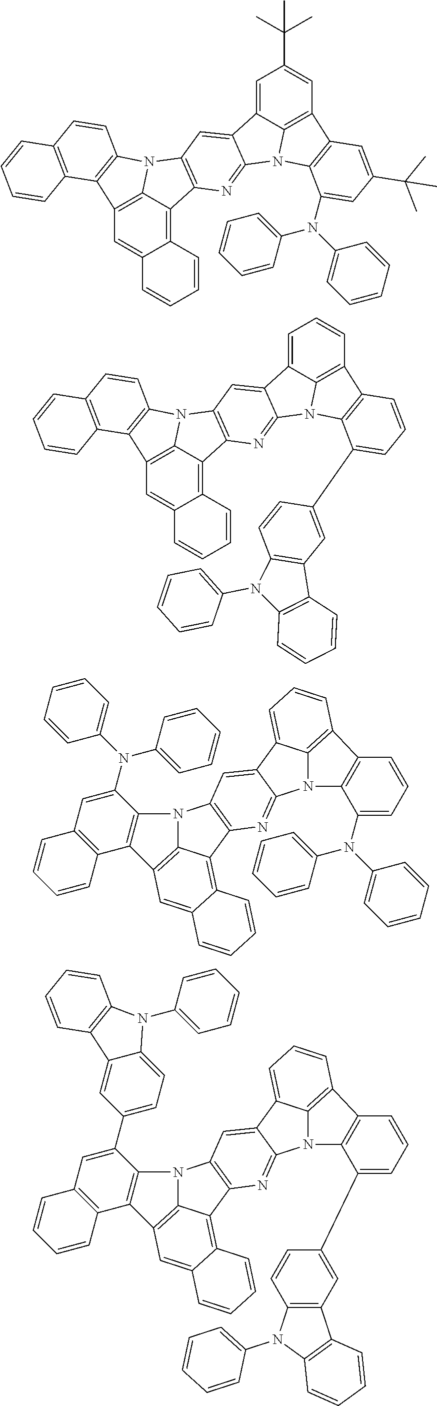

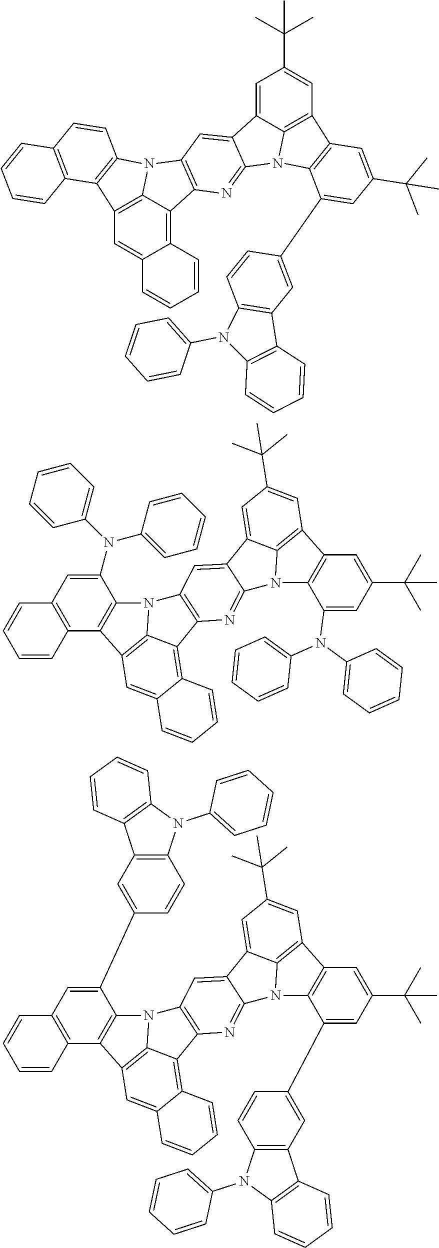

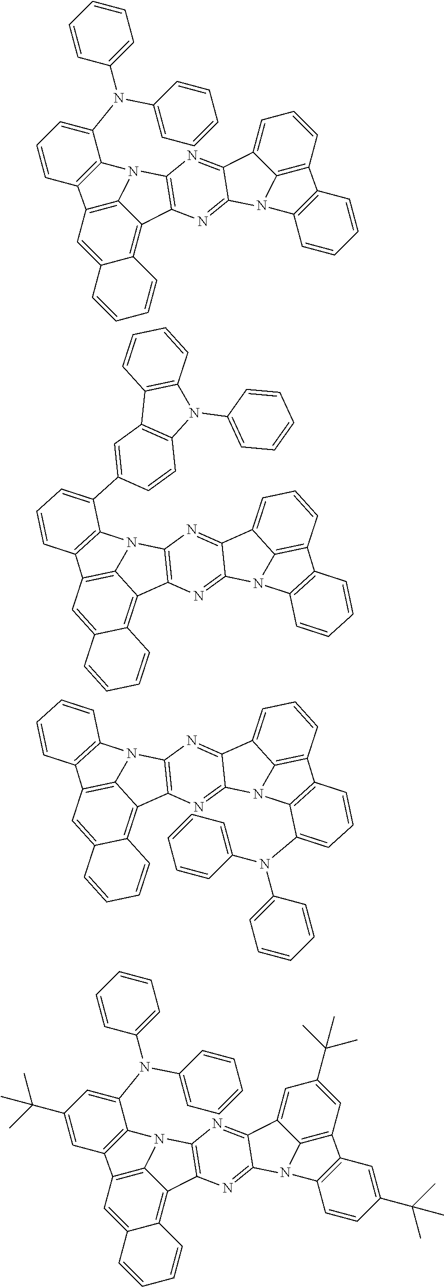

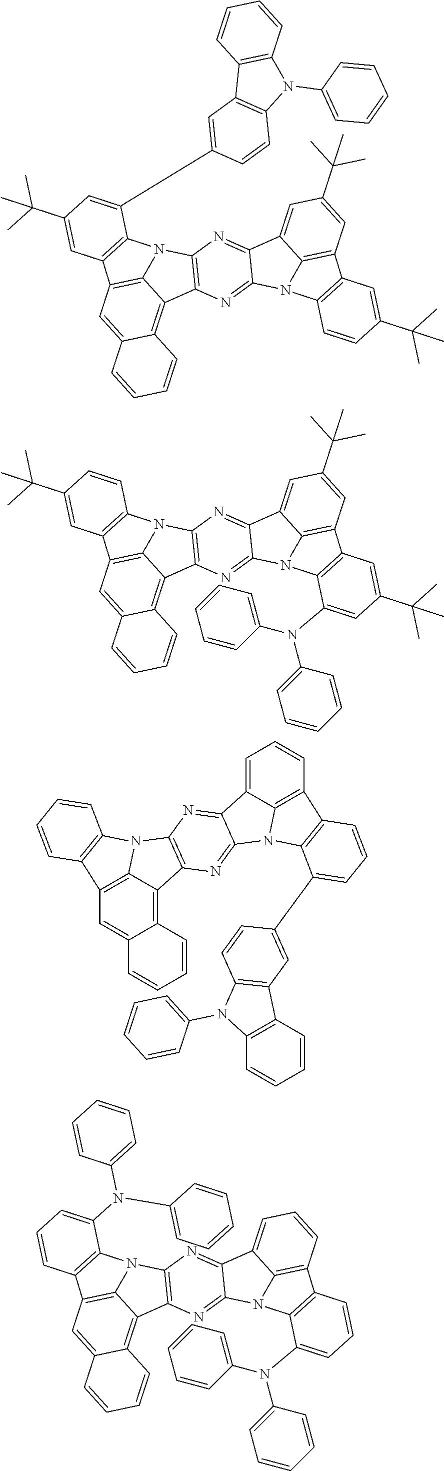

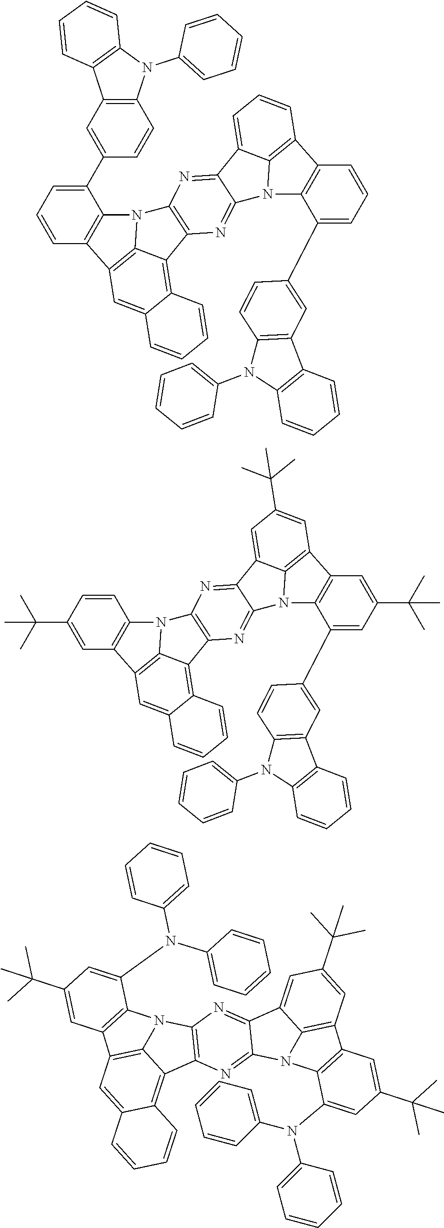

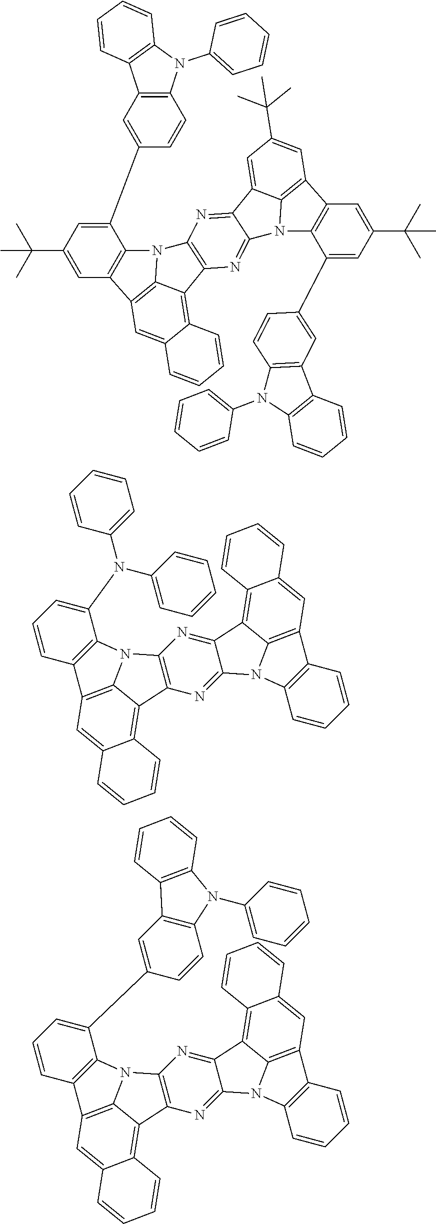

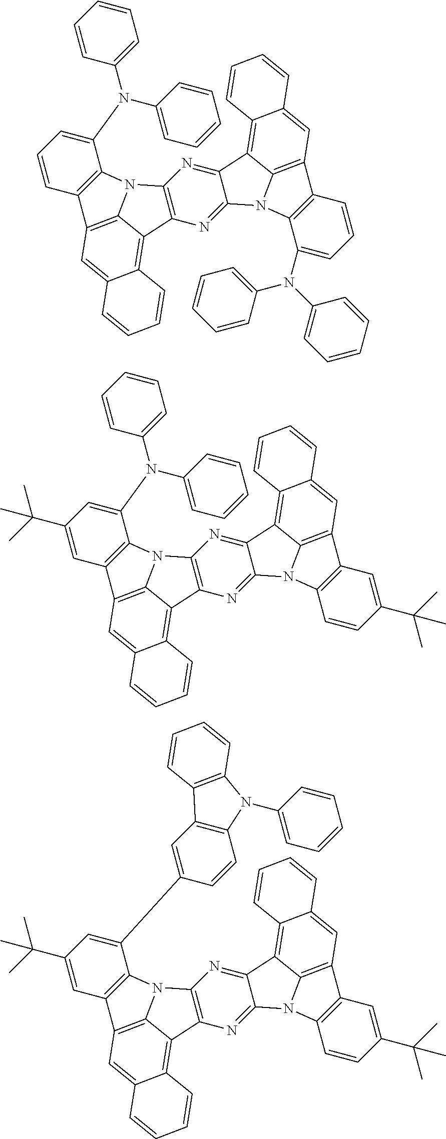

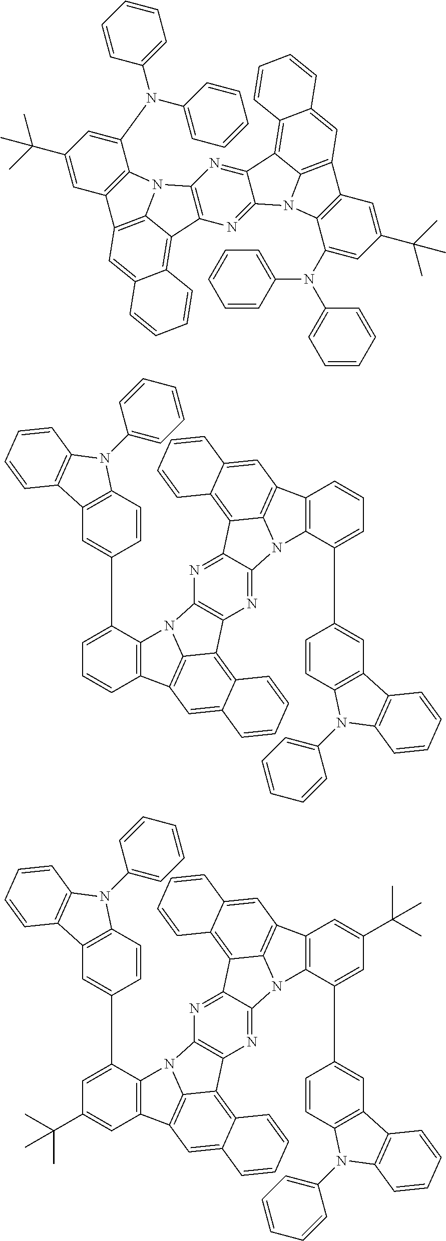

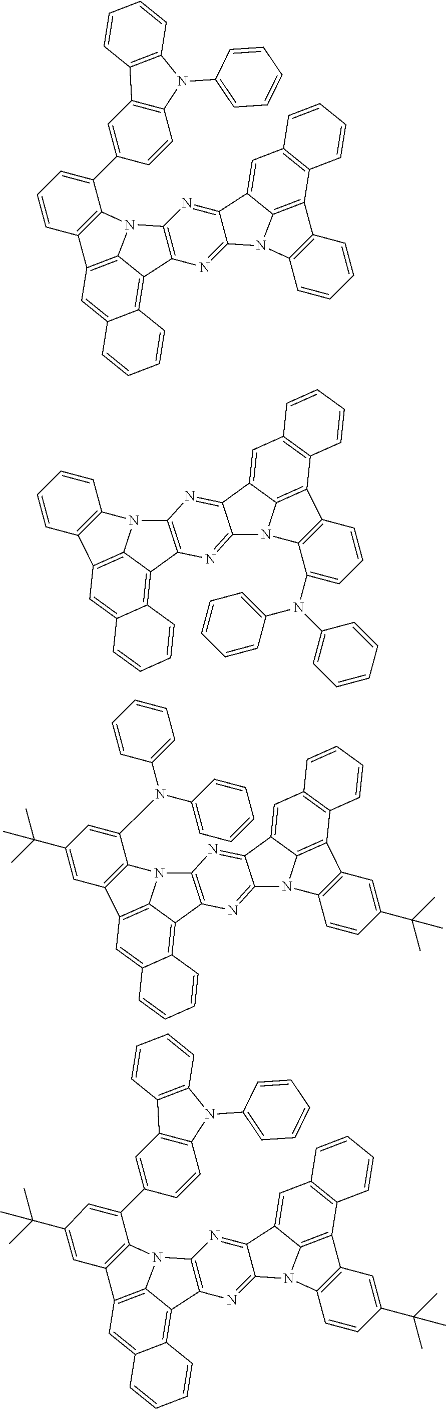

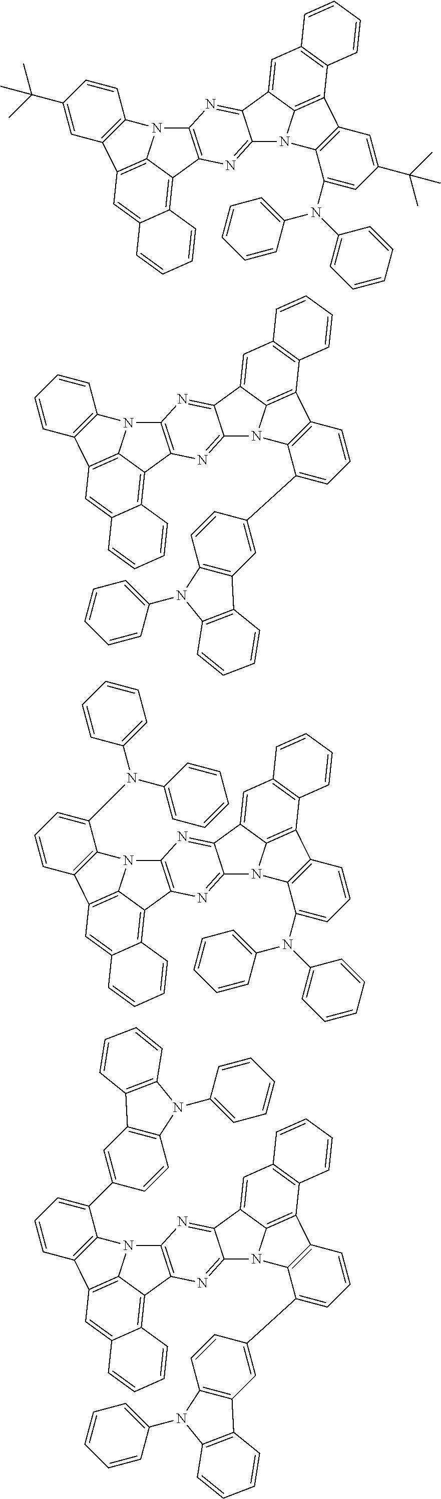

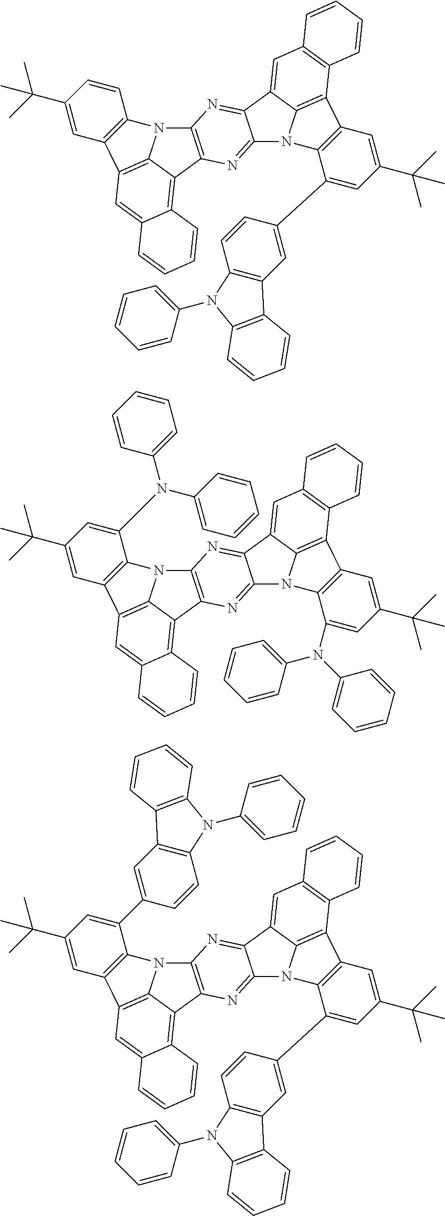

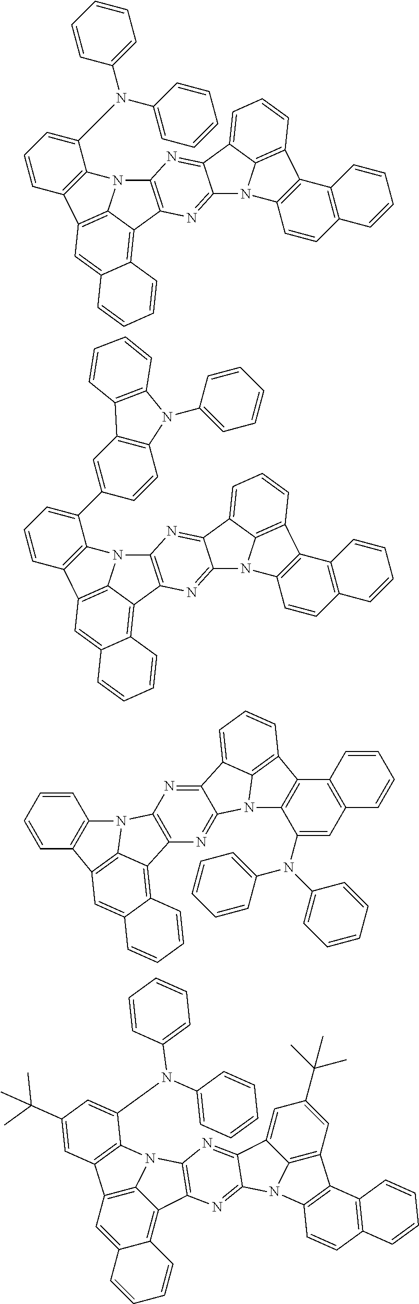

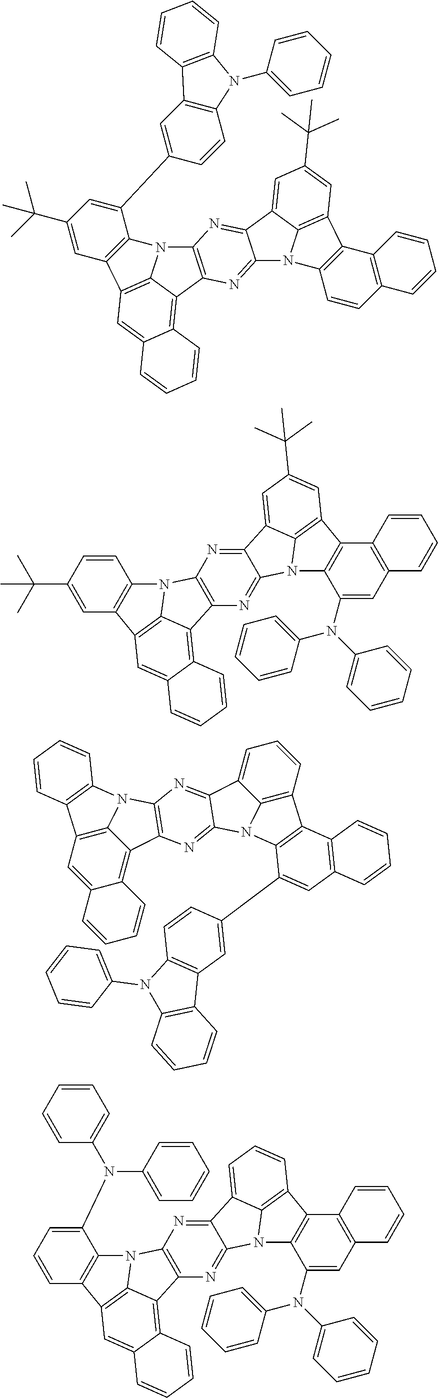 [SMILES: CC(C)(C)c1cc(-c2ccc3c(c2)c2ccccc2n3-c2ccccc2)c2c(c1)c1cc3ccccc3c3c4nc5c(nc4n2c13)c1cc(C(C)(C)C)cc2c3c4ccccc4ccc3n5c12 ; CC(C)(C)c1ccc2c(c1)c1cc3ccccc3c3c4nc5c(nc4n2c13)c1cc(C(C)(C)C)cc2c3c4ccccc4cc(N(c4ccccc4)c4ccccc4)c3n5c12 ; c1ccc(-n2c3ccccc3c3cc(-c4cc5ccccc5c5c6cccc7c8nc9c(nc8n(c45)c76)c4c5ccccc5cc5c6ccccc6n9c54)ccc32)cc1 ; c1ccc(N(c2ccccc2)c2cc3ccccc3c3c4cccc5c6nc7c(nc6n(c23)c54)c2c3ccccc3cc3c4cccc(N(c5ccccc5)c5ccccc5)c4n7c32)cc1]

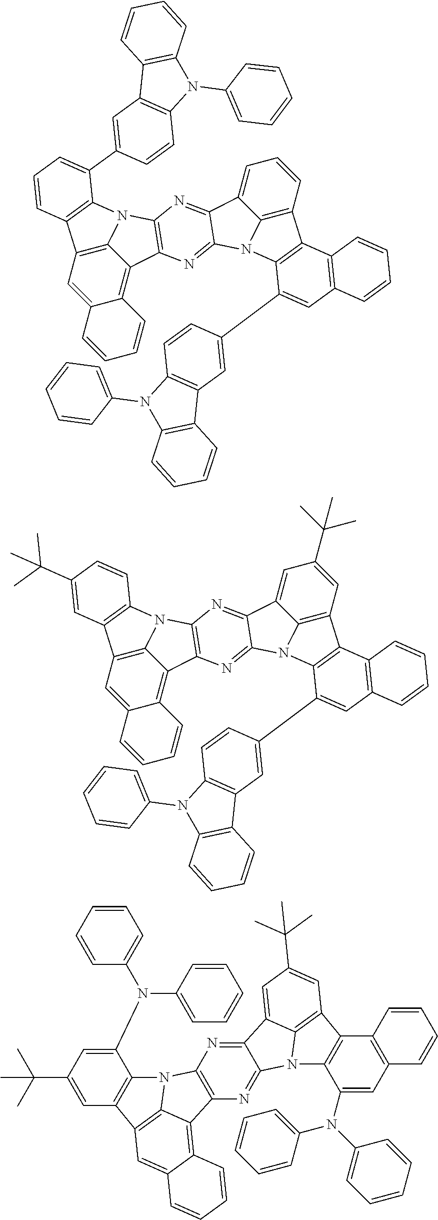

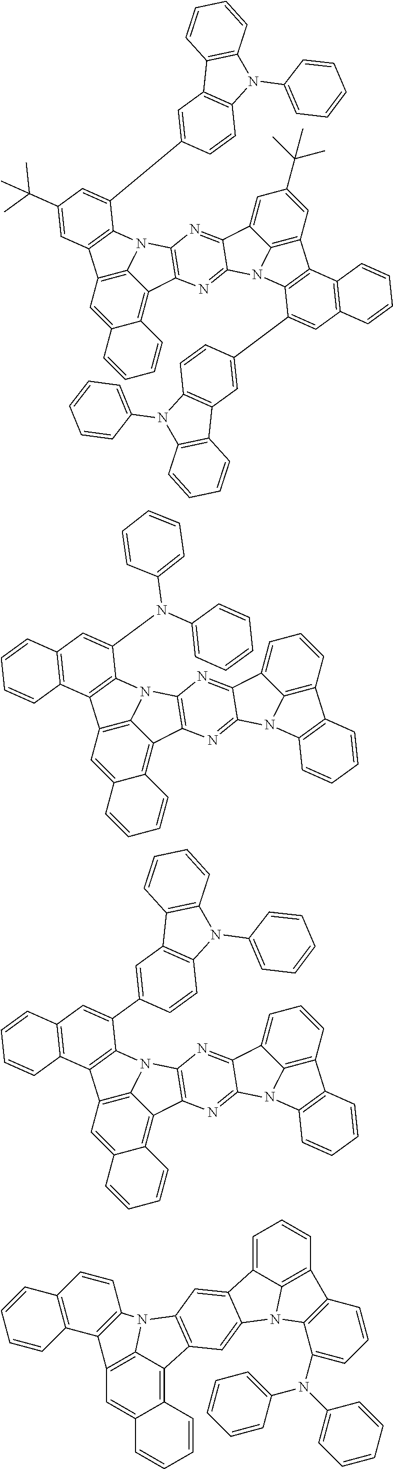

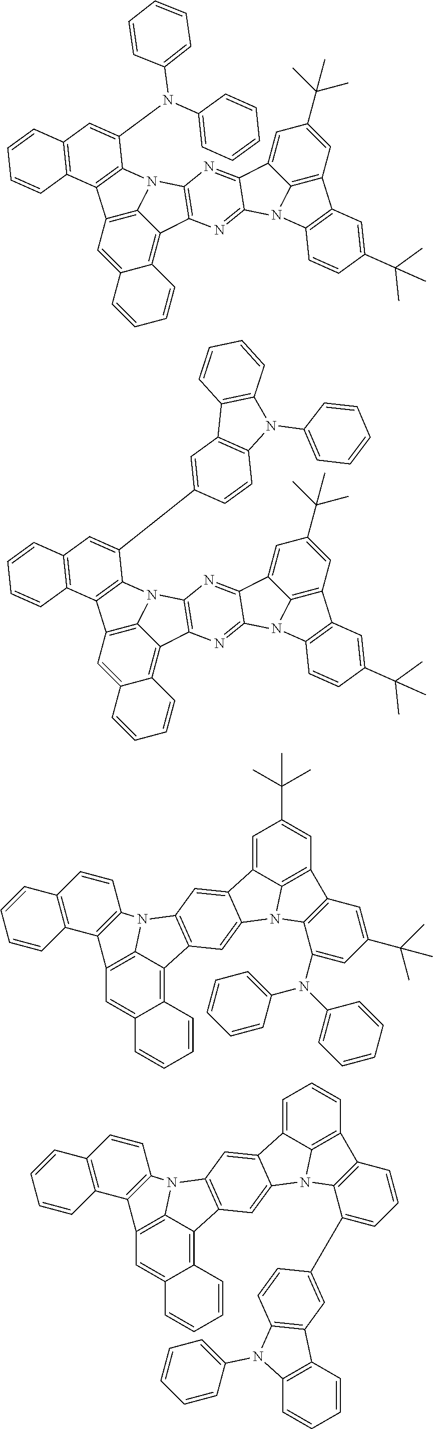

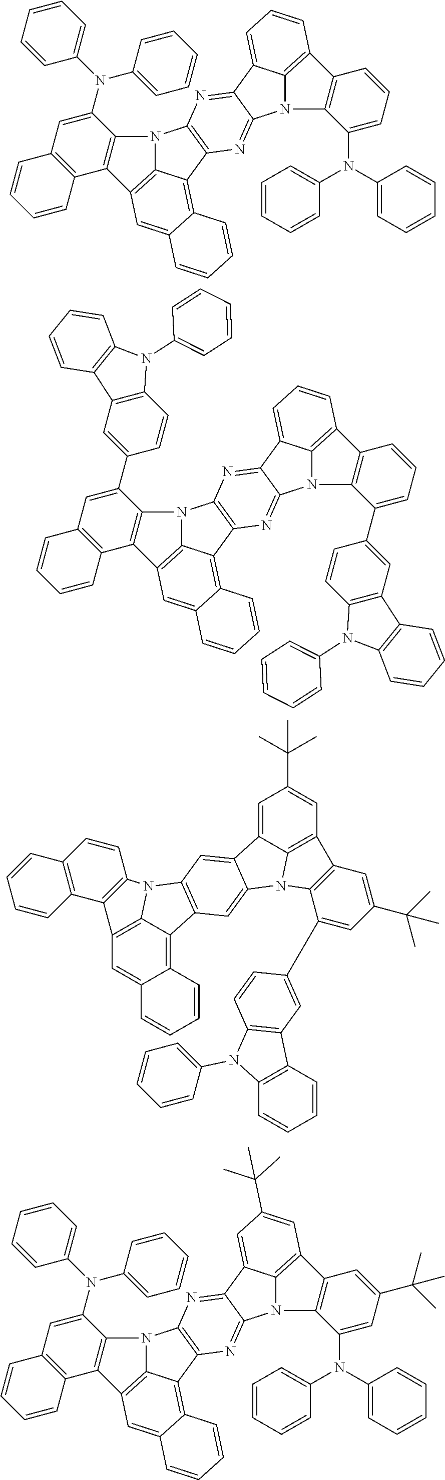

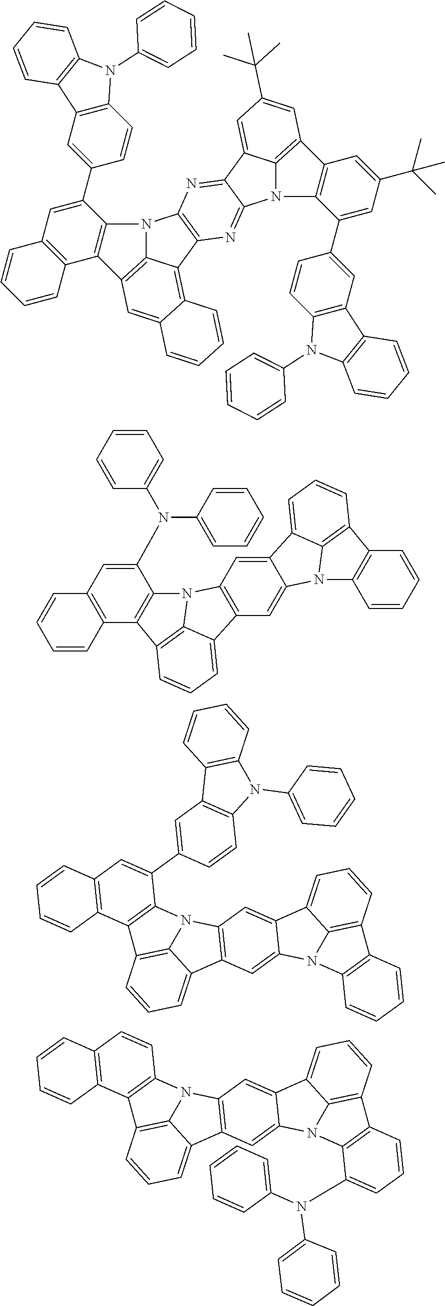

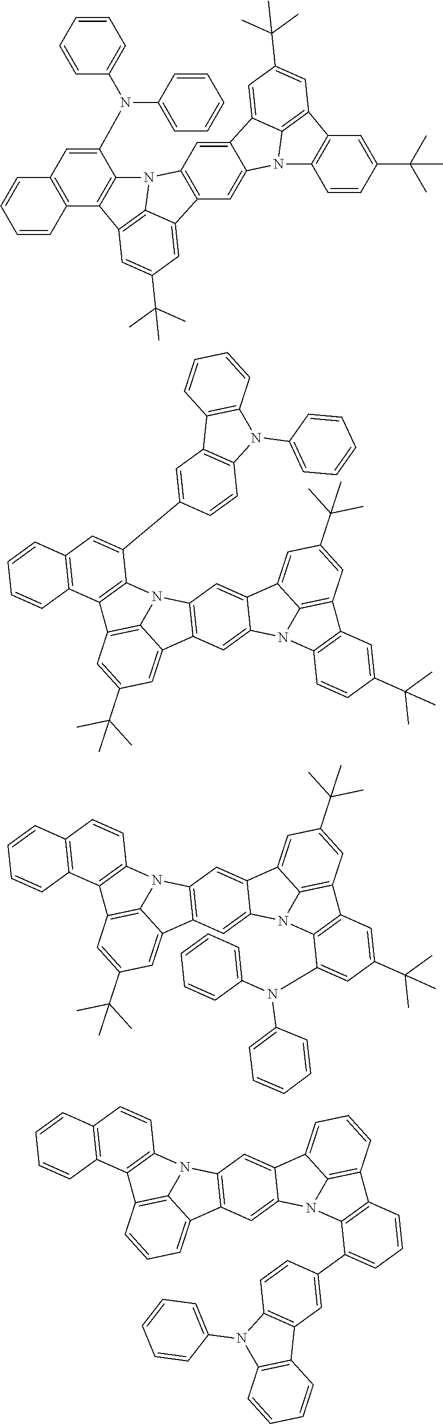

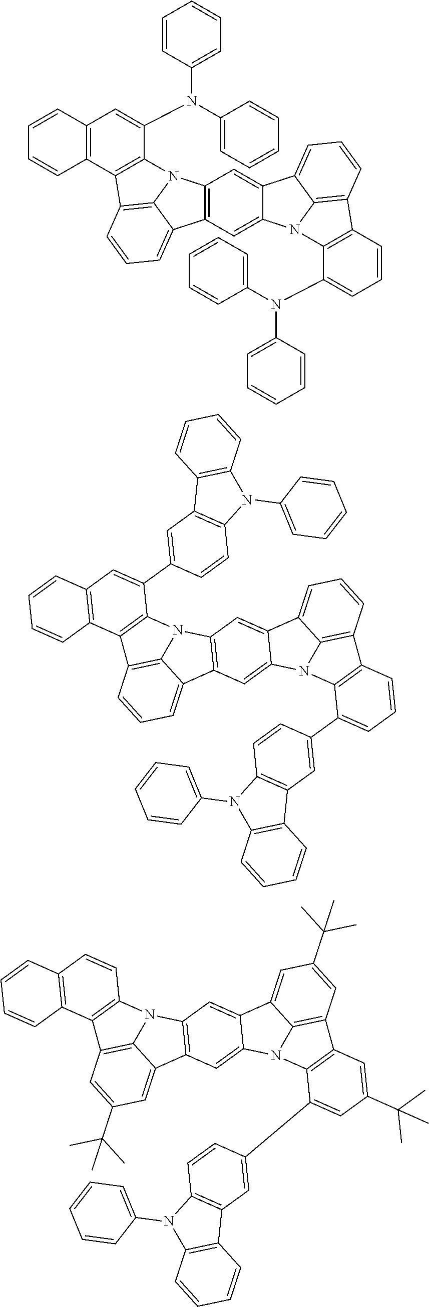

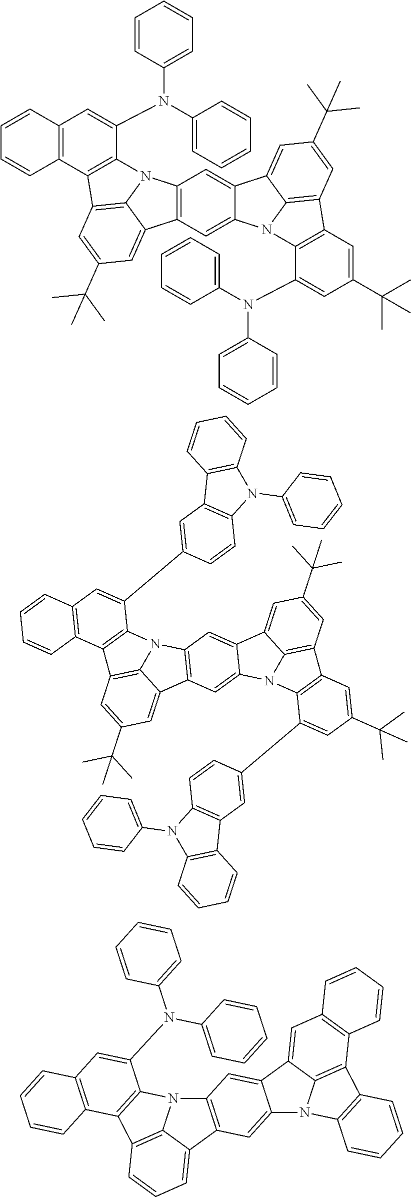

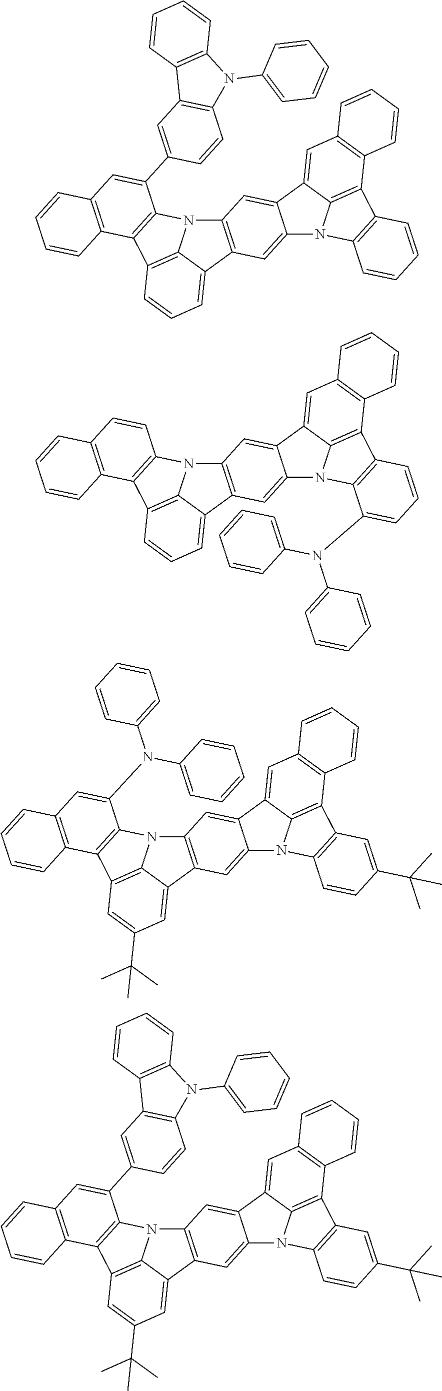

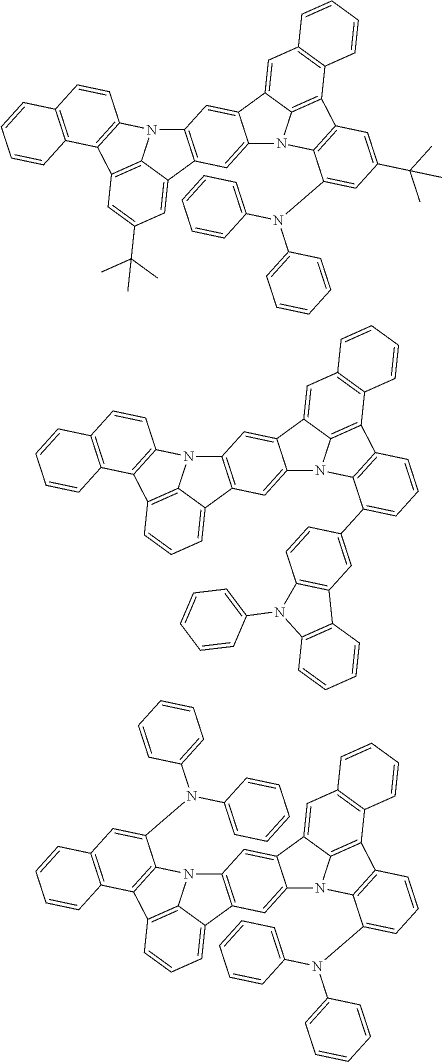

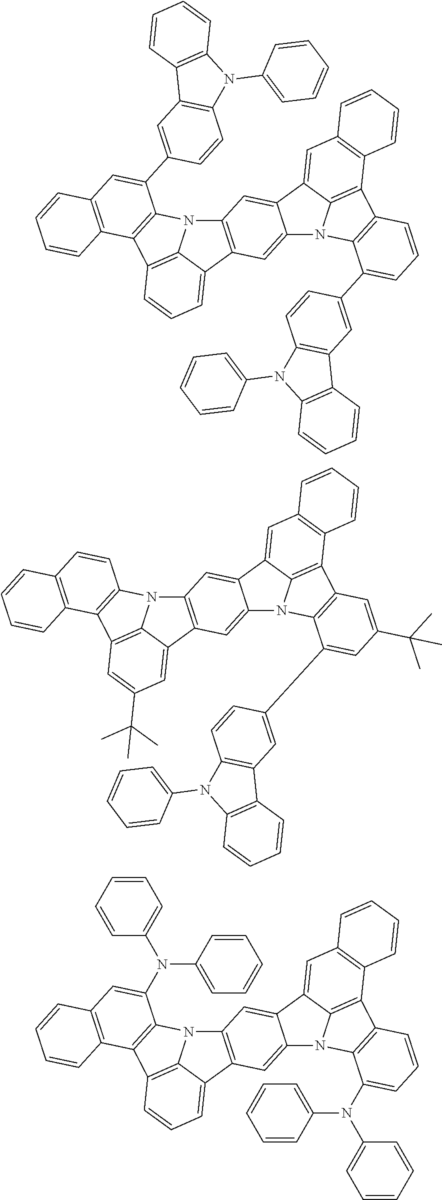

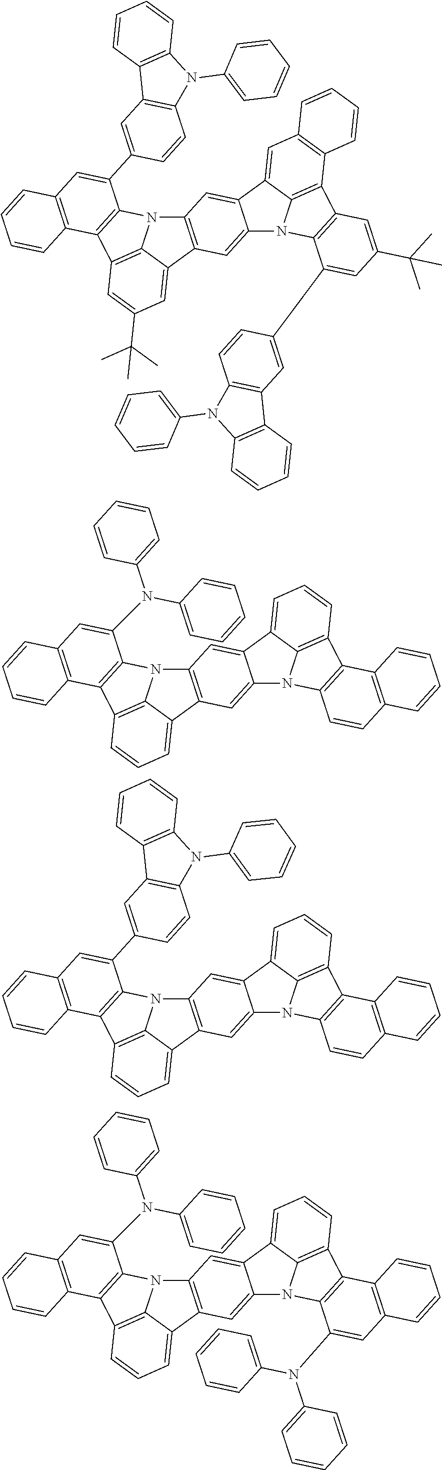

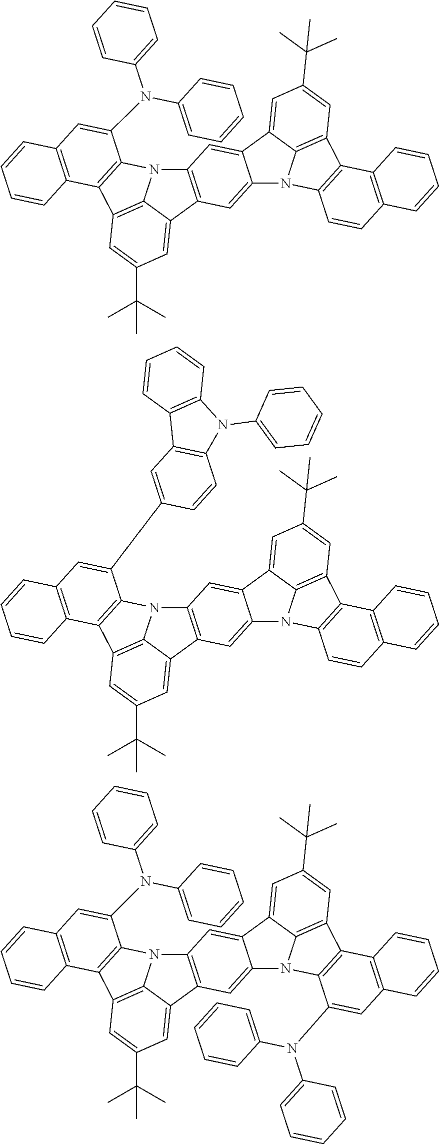

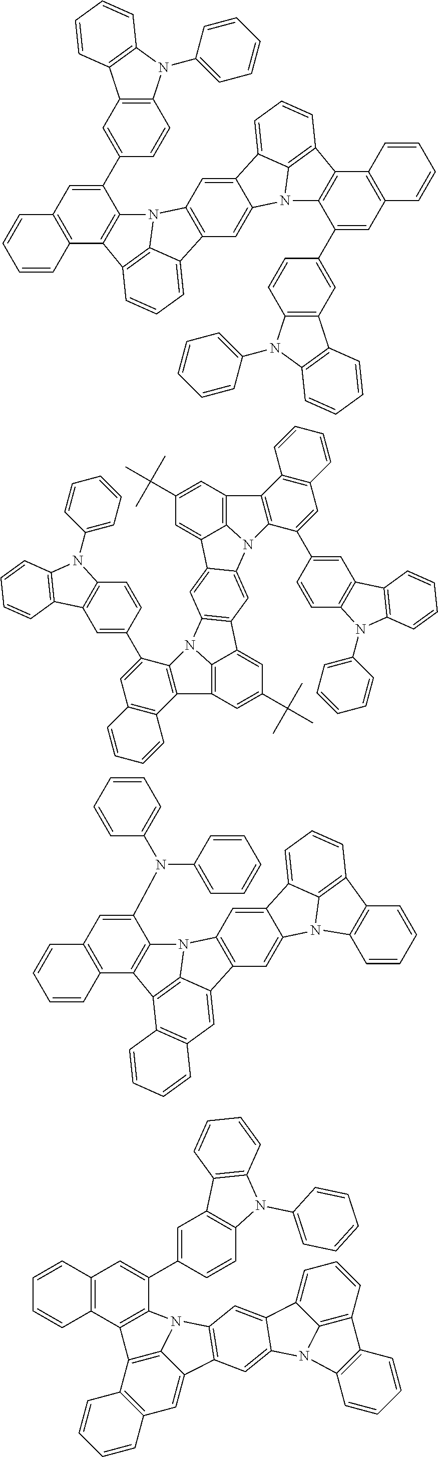

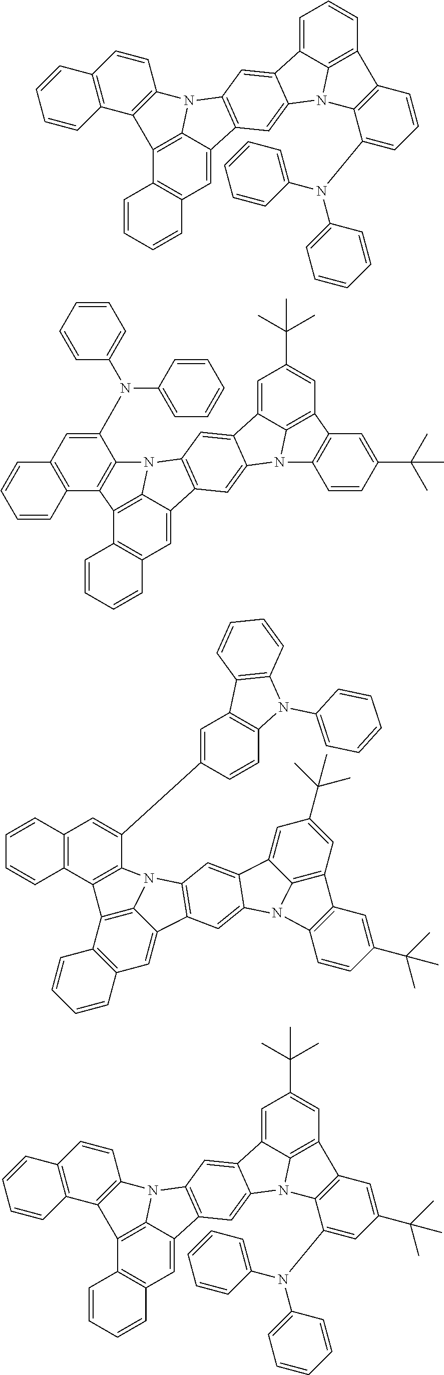

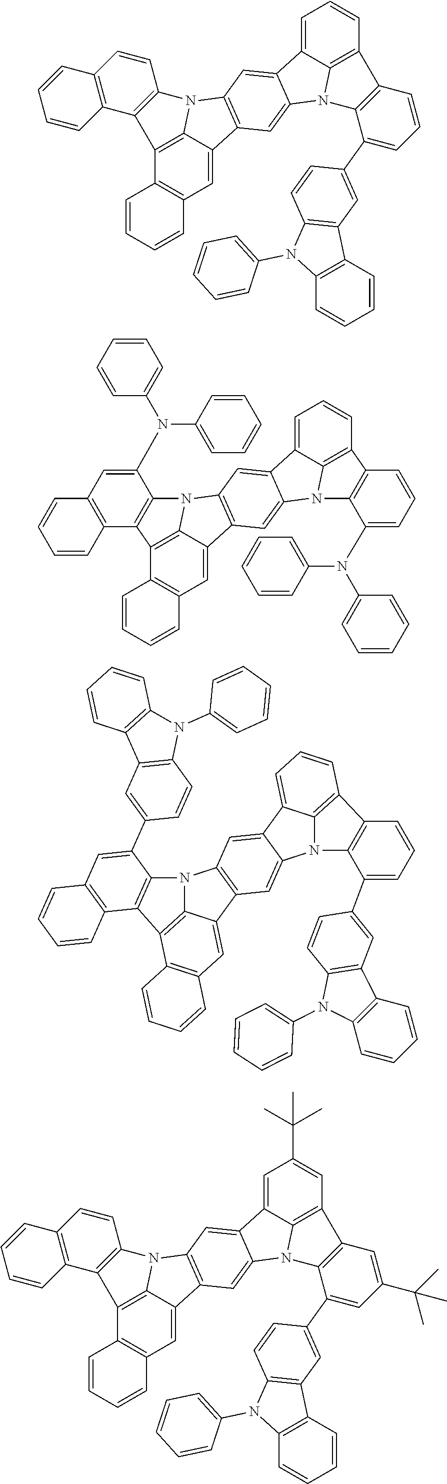

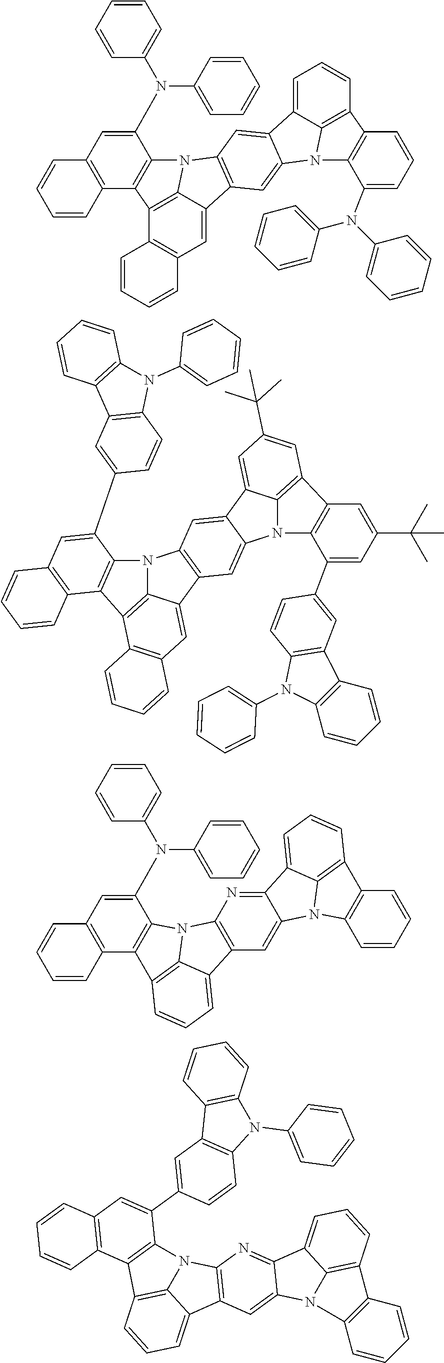

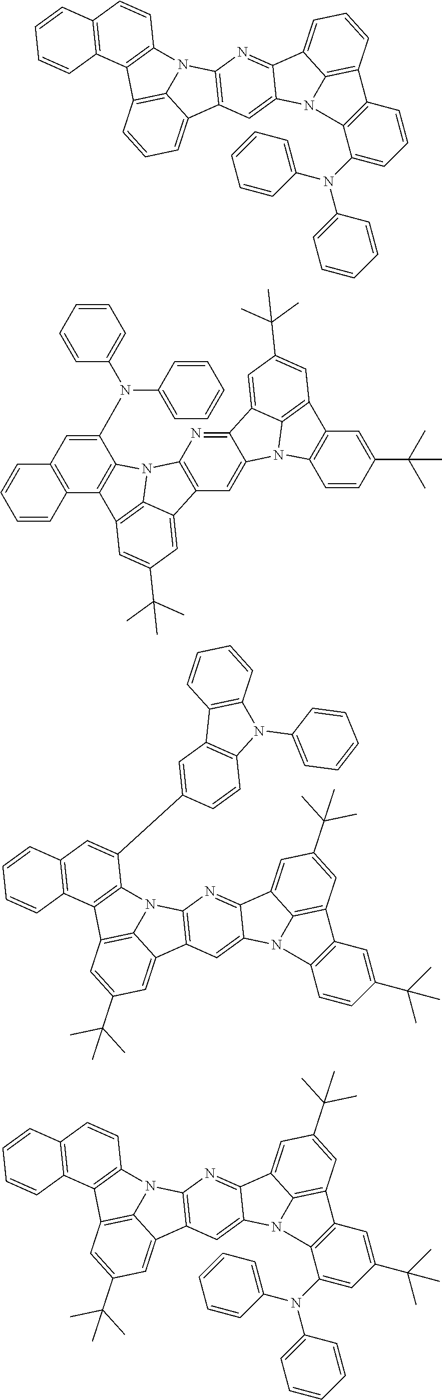

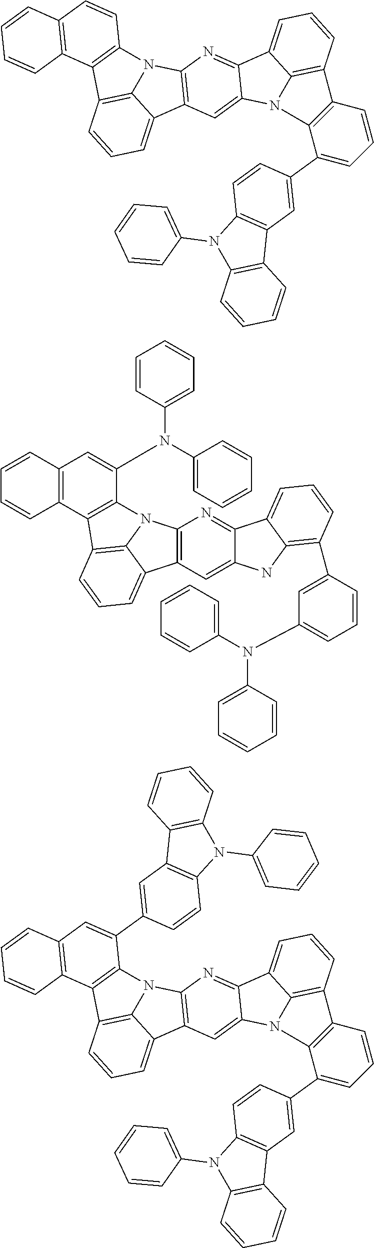

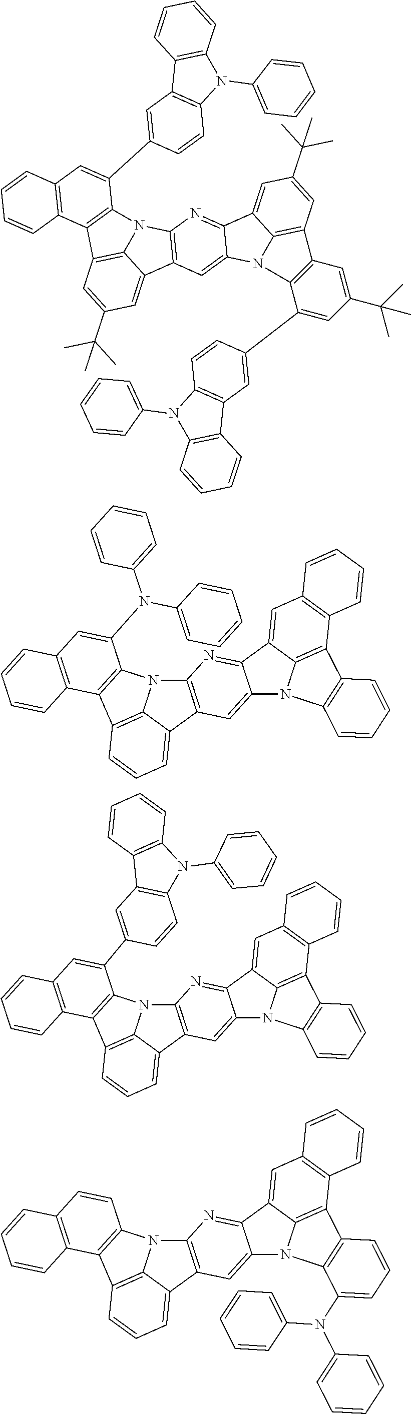

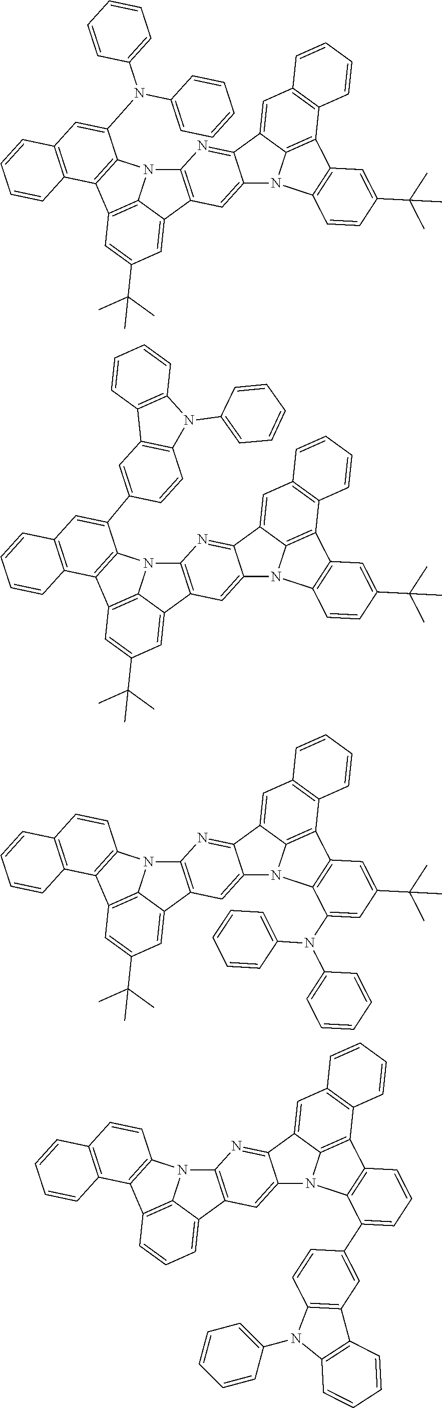

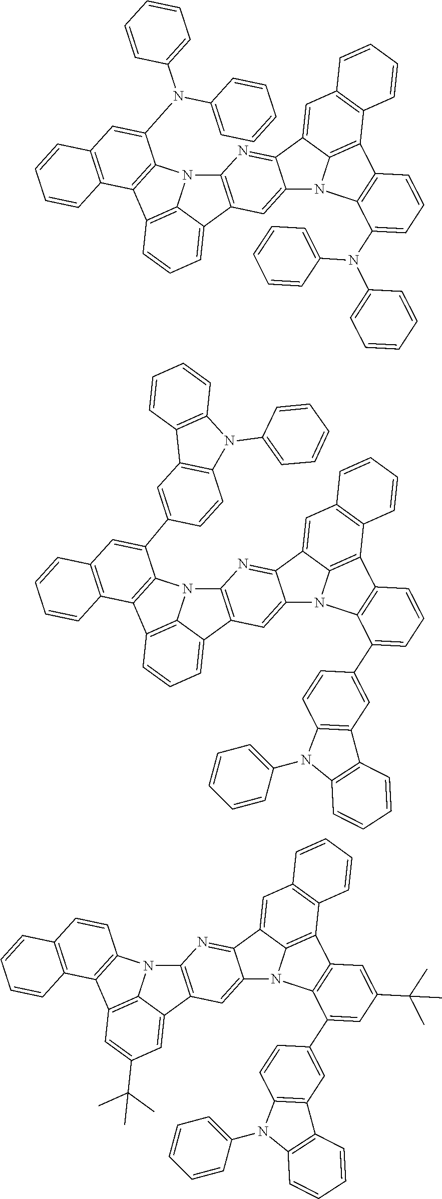

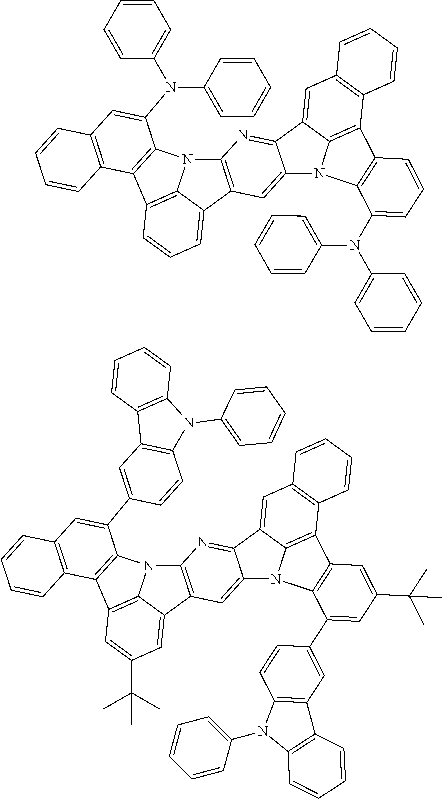 [SMILES: c1ccc(N(c2ccccc2)c2cccc3c4c5ccccc5cc5c6nc7c(cc6n(c23)c54)c2cccc3c4c5ccccc5cc(N(c5ccccc5)c5ccccc5)c4n7c23)cc1 ; CC(C)(C)c1cc(-c2ccc3c(c2)c2ccccc2n3-c2ccccc2)c2c(c1)c1c3ccccc3cc3c4nc5c(cc4n2c31)c1cc(C(C)(C)C)cc2c3c4ccccc4cc(-c4ccc6c(c4)c4ccccc4n6-c4ccccc4)c3n5c12]

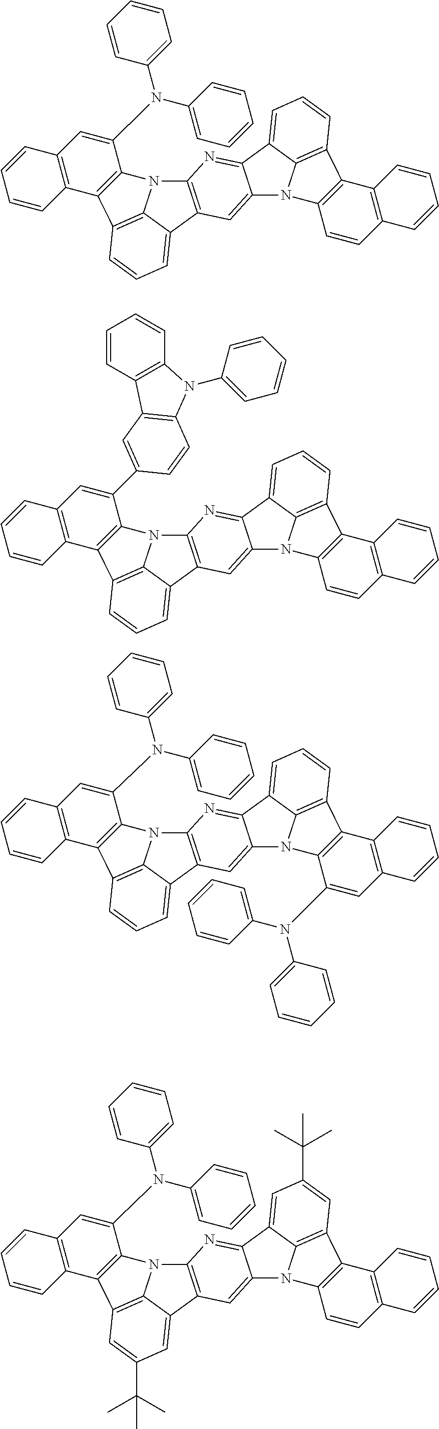

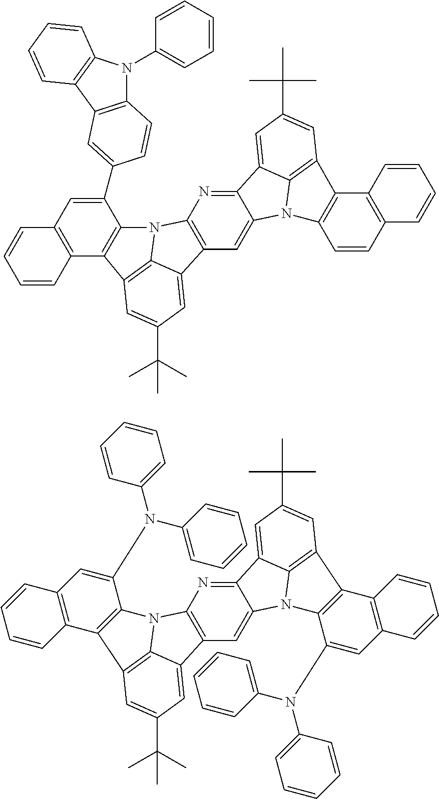

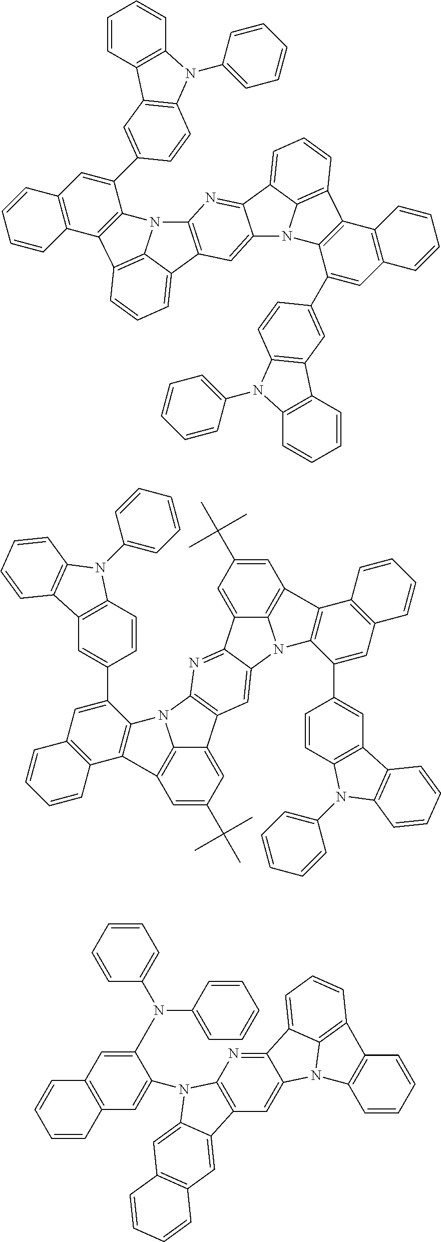 [SMILES: c1ccc(-n2c3ccccc3c3cc(-c4cc5ccccc5c5c6cccc7c8nc9c(cc8n(c45)c76)c4cccc5c6c7ccccc7cc(-c7ccc8c(c7)c7ccccc7n8-c7ccccc7)c6n9c45)ccc32)cc1 ; CC(C)(C)c1cc2c3nc4c(cc3n3c5c(-c6ccc7c(c6)c6ccccc6n7-c6ccccc6)cc6ccccc6c5c(c1)c23)c1cc(C(C)(C)C)cc2c3c5ccccc5cc(-c5ccc6c(c5)c5ccccc5n6-c5ccccc5)c3n4c12 ; c1ccc(N(c2ccccc2)c2cc3ccccc3cc2-n2c3cc4ccccc4cc3c3cc4c(nc32)c2cccc3c5ccccc5n4c32)cc1]

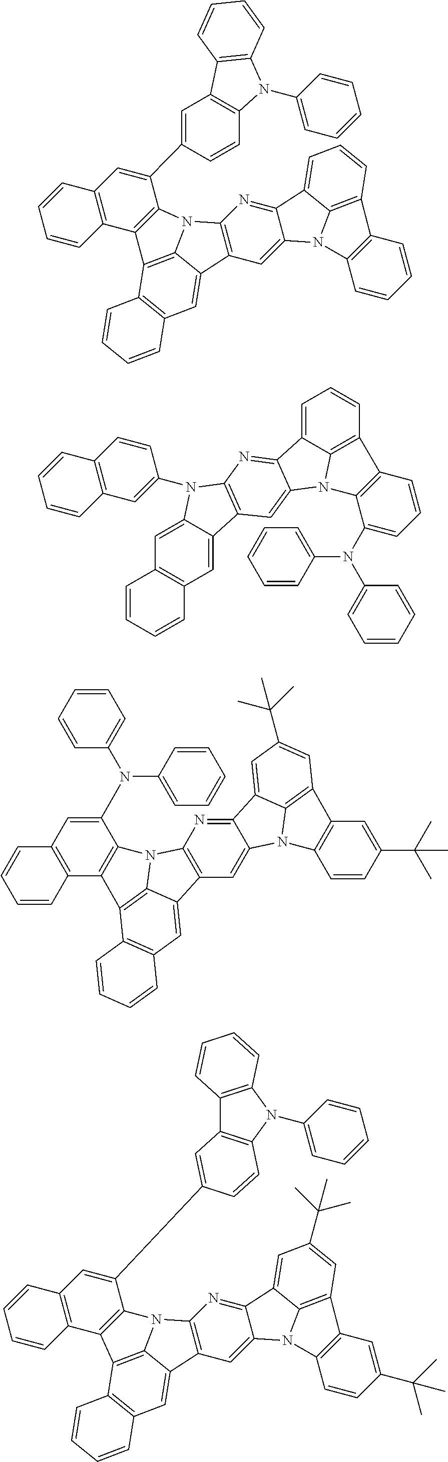 [SMILES: c1ccc(-n2c3ccccc3c3cc(-c4cc5ccccc5c5c6c7ccccc7cc7c8cc9c(nc8n(c45)c76)c4cccc5c6ccccc6n9c54)ccc32)cc1 ; c1ccc(N(c2ccccc2)c2cccc3c4cccc5c6nc7c(cc6n(c23)c45)c2cc3ccccc3cc2n7-c2ccc3ccccc3c2)cc1 ; CC(C)(C)c1ccc2c(c1)c1cc(C(C)(C)C)cc3c4nc5c(cc4n2c13)c1cc2ccccc2c2c3c4ccccc4cc(N(c4ccccc4)c4ccccc4)c3n5c12 ; CC(C)(C)c1ccc2c(c1)c1cc(C(C)(C)C)cc3c4nc5c(cc4n2c13)c1cc2ccccc2c2c3c4ccccc4cc(-c4ccc6c(c4)c4ccccc4n6-c4ccccc4)c3n5c12]

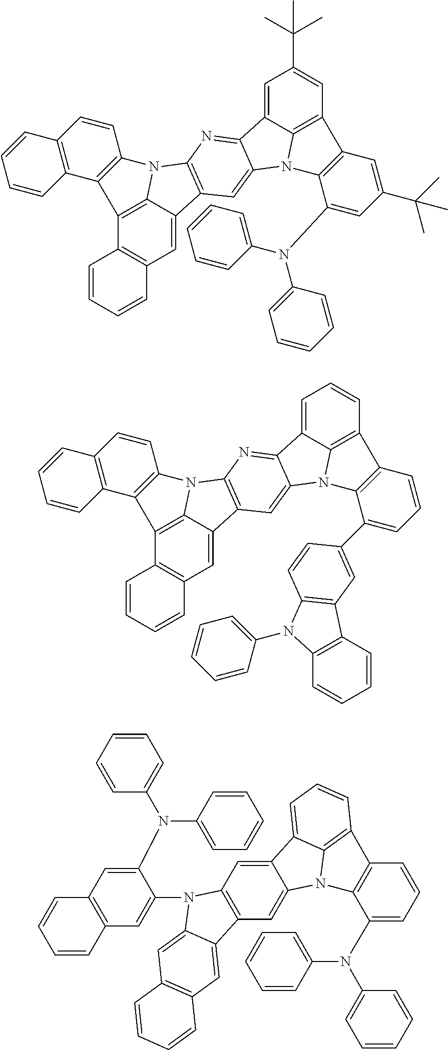

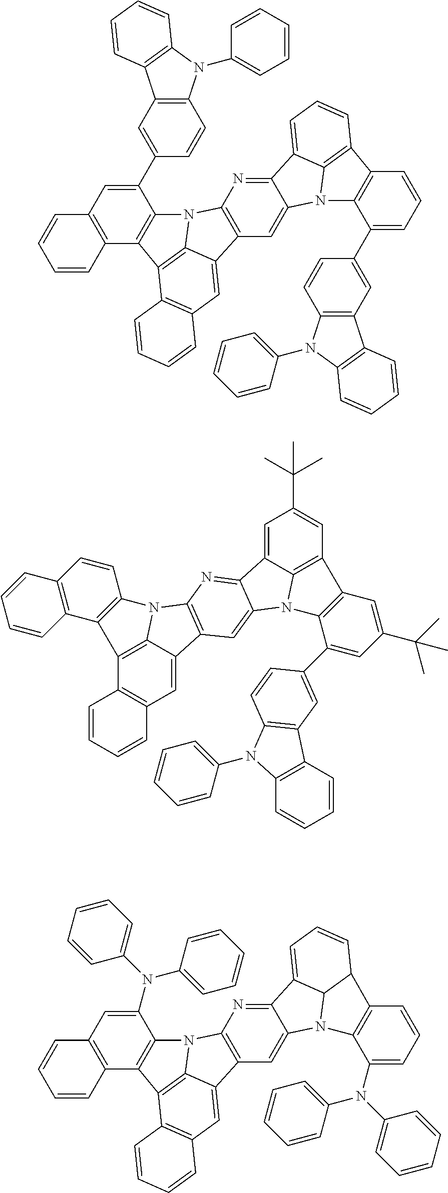 [SMILES: c1ccc(-n2c3ccccc3c3cc(-c4cccc5c6cccc7c8nc9c(cc8n(c45)c67)c4cc5ccccc5c5c6c7ccccc7cc(-c7ccc8c(c7)c7ccccc7n8-c7ccccc7)c6n9c45)ccc32)cc1 ; CC(C)(C)c1cc(-c2ccc3c(c2)c2ccccc2n3-c2ccccc2)c2c(c1)c1cc(C(C)(C)C)cc3c4nc5c(cc4n2c13)c1cc2ccccc2c2c3c4ccccc4ccc3n5c12 ; C1=CC2c3cccc(N(c4ccccc4)c4ccccc4)c3N3c4cc5c6cc7ccccc7c7c8c9ccccc9cc(N(c9ccccc9)c9ccccc9)c8n(c5nc4C(=C1)C23)c67]

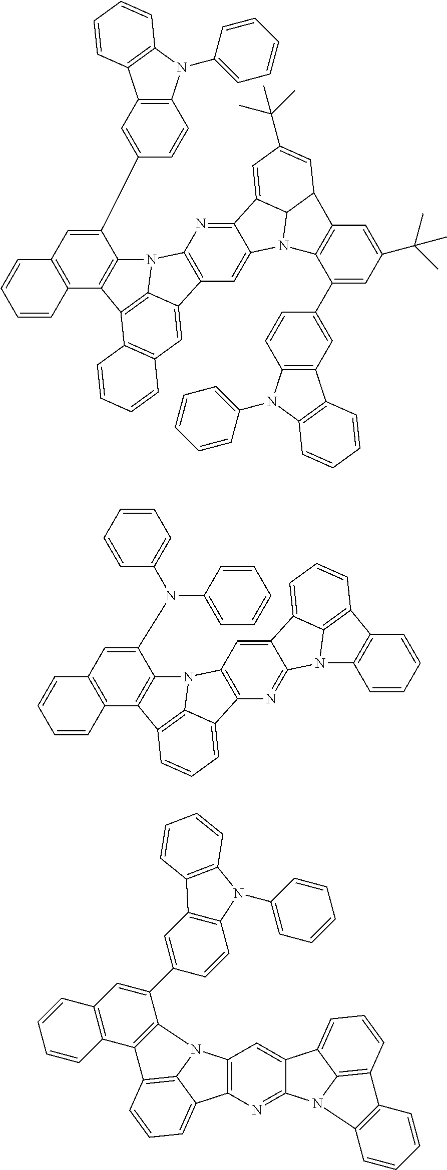

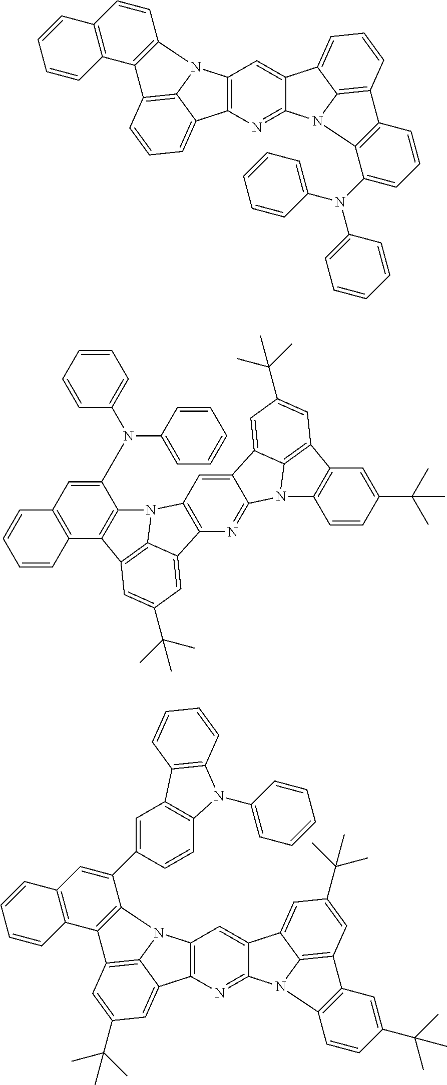

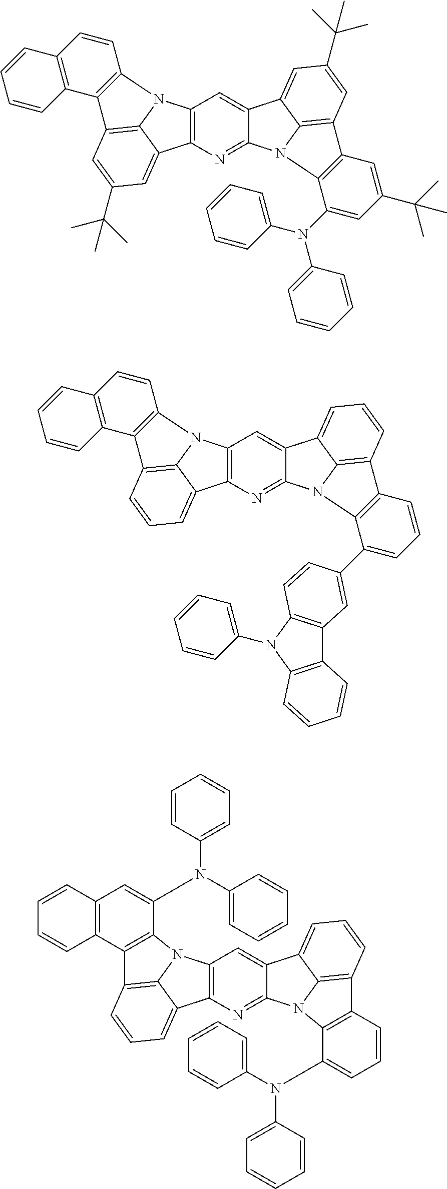 [SMILES: CC(C)(C)c1cc(N(c2ccccc2)c2ccccc2)c2c(c1)c1cc(C(C)(C)C)cc3c4cc5c(nc4n2c31)c1cc(C(C)(C)C)cc2c3c4ccccc4ccc3n5c12 ; c1ccc(-n2c3ccccc3c3cc(-c4cccc5c6cccc7c8cc9c(nc8n(c45)c76)c4cccc5c6c7ccccc7ccc6n9c45)ccc32)cc1 ; c1ccc(N(c2ccccc2)c2cc3ccccc3c3c4cccc5c6nc7c(cc6n(c23)c54)c2cccc3c4cccc(N(c5ccccc5)c5ccccc5)c4n7c23)cc1]

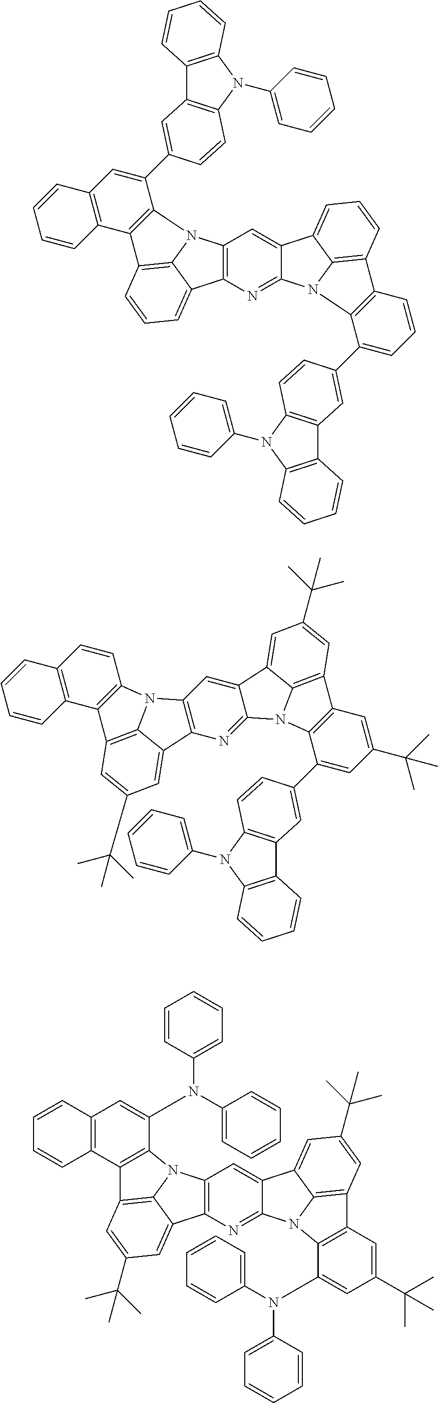

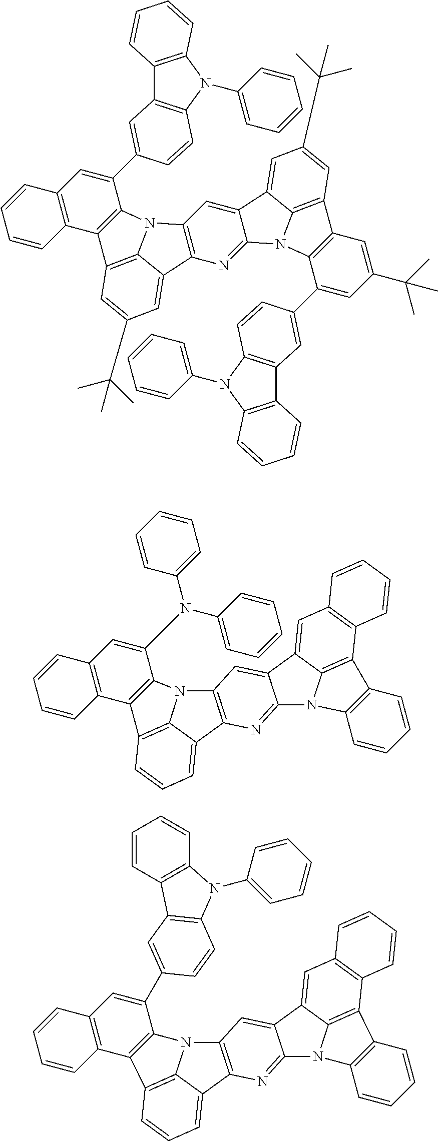

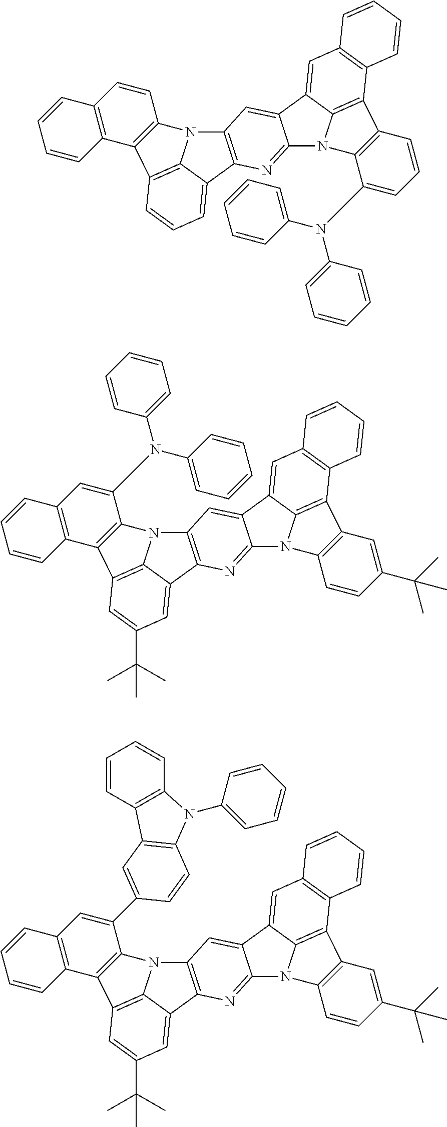

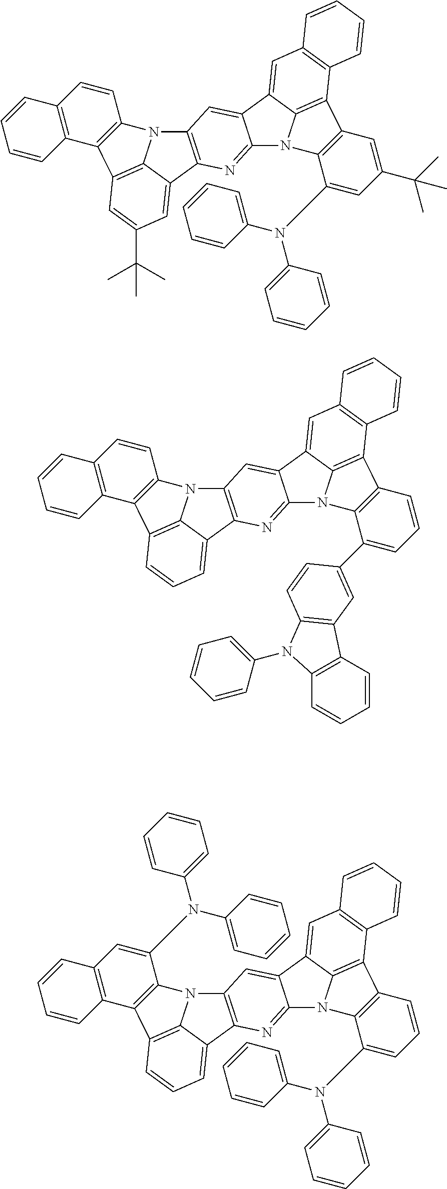 [SMILES: CC(C)(C)c1cc(N(c2ccccc2)c2ccccc2)c2c(c1)c1c3ccccc3cc3c4cc5c(nc4n2c31)c1cc(C(C)(C)C)cc2c3c4ccccc4ccc3n5c12 ; c1ccc(-n2c3ccccc3c3cc(-c4cccc5c6c7ccccc7cc7c8cc9c(nc8n(c45)c76)c4cccc5c6c7ccccc7ccc6n9c45)ccc32)cc1 ; c1ccc(N(c2ccccc2)c2cc3ccccc3c3c4cccc5c6nc7c(cc6n(c23)c54)c2cc3ccccc3c3c4cccc(N(c5ccccc5)c5ccccc5)c4n7c23)cc1]

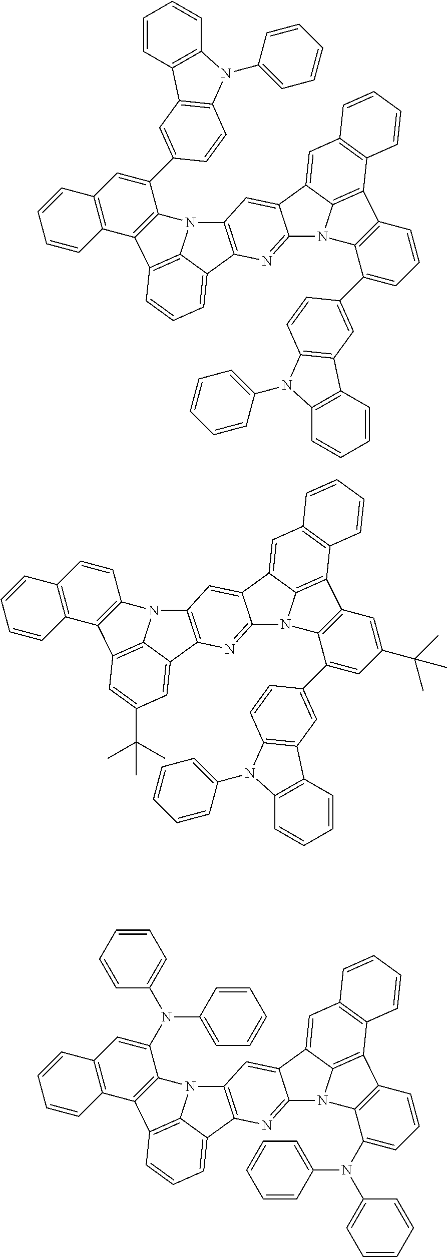

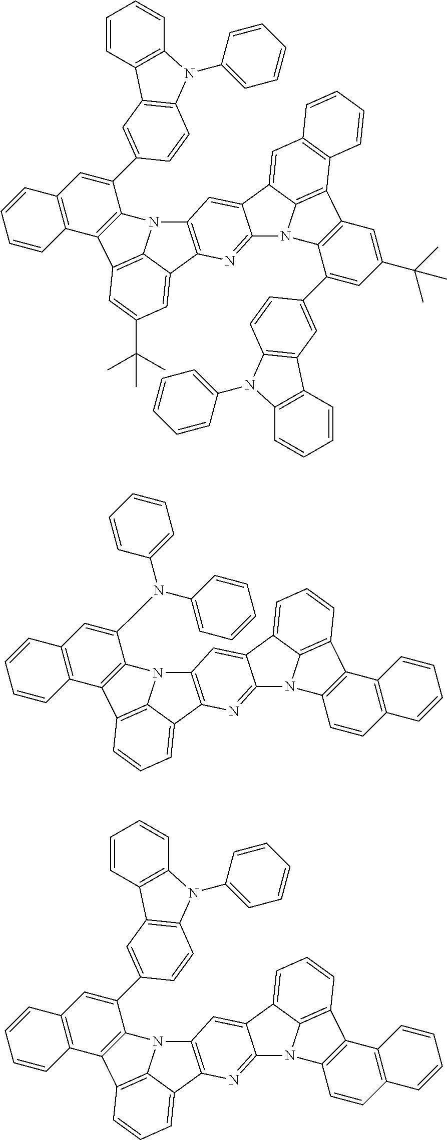 [SMILES: CC(C)(C)c1cc(-c2ccc3c(c2)c2ccccc2n3-c2ccccc2)c2c(c1)c1c3ccccc3cc3c4cc5c(nc4n2c31)c1cc(C(C)(C)C)cc2c3c4ccccc4cc(-c4ccc6c(c4)c4ccccc4n6-c4ccccc4)c3n5c12 ; c1ccc(N(c2ccccc2)c2cc3ccccc3c3c4cccc5c6nc7c(cc6n(c23)c54)c2cccc3c4c5ccccc5ccc4n7c23)cc1 ; c1ccc(-n2c3ccccc3c3cc(-c4cc5ccccc5c5c6cccc7c8nc9c(cc8n(c45)c76)c4cccc5c6c7ccccc7ccc6n9c45)ccc32)cc1]

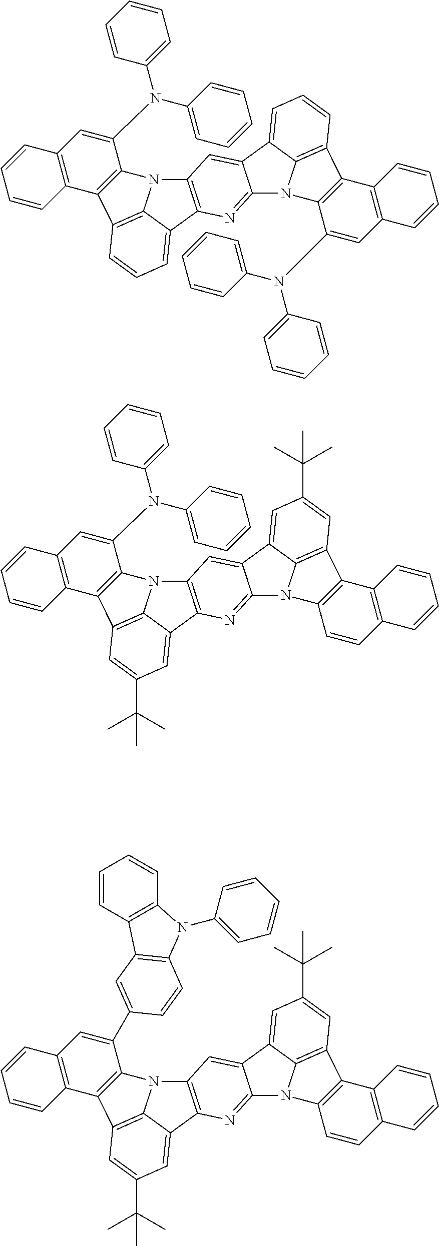 [SMILES: c1ccc(N(c2ccccc2)c2cc3ccccc3c3c4cccc5c6nc7c(cc6n(c23)c54)c2cccc3c4c5ccccc5cc(N(c5ccccc5)c5ccccc5)c4n7c23)cc1 ; CC(C)(C)c1cc2c3cc4c(nc3n3c5ccc6ccccc6c5c(c1)c23)c1cc(C(C)(C)C)cc2c3c5ccccc5cc(N(c5ccccc5)c5ccccc5)c3n4c12 ; CC(C)(C)c1cc2c3cc4c(nc3n3c5ccc6ccccc6c5c(c1)c23)c1cc(C(C)(C)C)cc2c3c5ccccc5cc(-c5ccc6c(c5)c5ccccc5n6-c5ccccc5)c3n4c12]

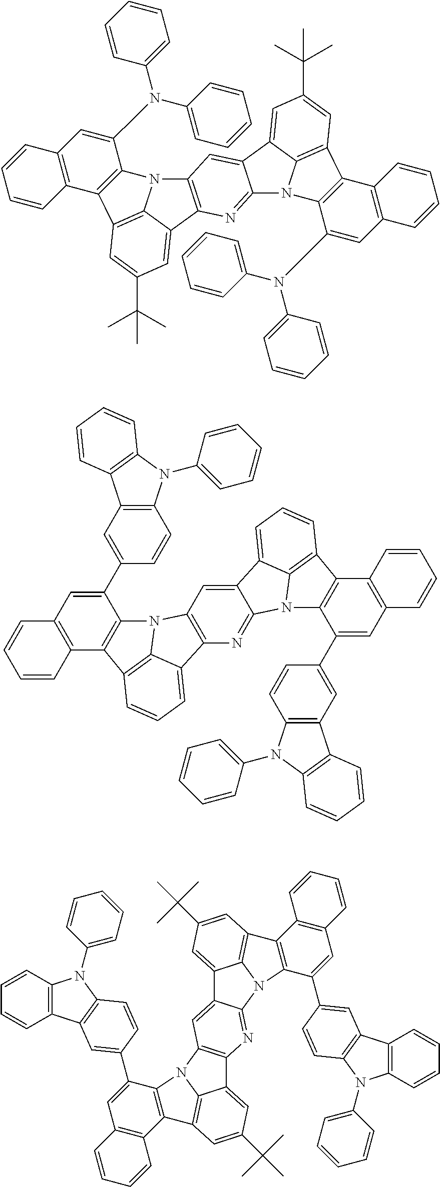 [SMILES: CC(C)(C)c1cc2c3nc4c(cc3n3c5c(N(c6ccccc6)c6ccccc6)cc6ccccc6c5c(c1)c23)c1cc(C(C)(C)C)cc2c3c5ccccc5cc(N(c5ccccc5)c5ccccc5)c3n4c12 ; c1ccc(-n2c3ccccc3c3cc(-c4cc5ccccc5c5c6cccc7c8nc9c(cc8n(c45)c76)c4cccc5c6c7ccccc7cc(-c7ccc8c(c7)c7ccccc7n8-c7ccccc7)c6n9c45)ccc32)cc1 ; CC(C)(C)c1cc2c3nc4c(cc3n3c5c(-c6ccc7c(c6)c6ccccc6n7-c6ccccc6)cc6ccccc6c5c(c1)c23)c1cc(C(C)(C)C)cc2c3c5ccccc5cc(-c5ccc6c(c5)c5ccccc5n6-c5ccccc5)c3n4c12]

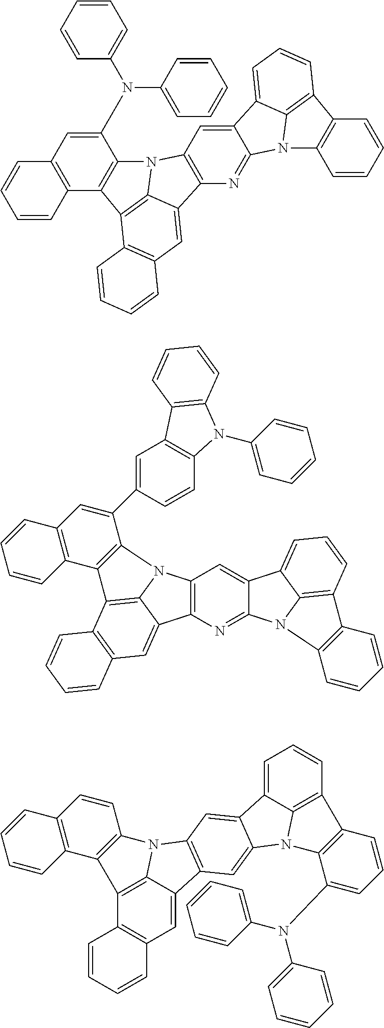 [SMILES: c1ccc(N(c2ccccc2)c2cc3ccccc3c3c4c5ccccc5cc5c6nc7c(cc6n(c23)c54)c2cccc3c4ccccc4n7c32)cc1 ; c1ccc(-n2c3ccccc3c3cc(-c4cc5ccccc5c5c6c7ccccc7cc7c8nc9c(cc8n(c45)c76)c4cccc5c6ccccc6n9c54)ccc32)cc1 ; c1ccc(N(c2ccccc2)c2cccc3c4cccc5c6cc7c(cc6n(c23)c54)c2cc3ccccc3c3c4c5ccccc5ccc4n7c23)cc1]

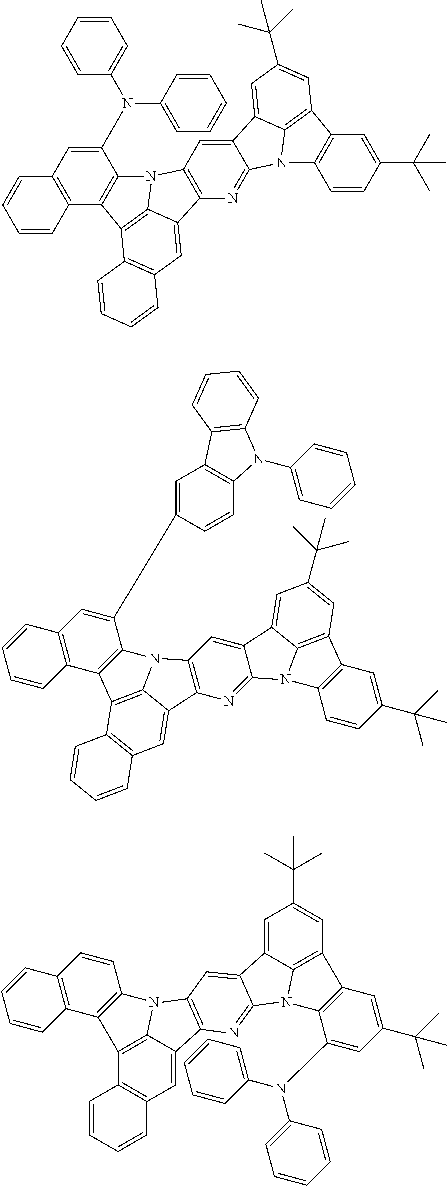 [SMILES: CC(C)(C)c1ccc2c(c1)c1cc(C(C)(C)C)cc3c4cc5c(nc4n2c13)c1cc2ccccc2c2c3c4ccccc4cc(N(c4ccccc4)c4ccccc4)c3n5c12 ; CC(C)(C)c1ccc2c(c1)c1cc(C(C)(C)C)cc3c4cc5c(nc4n2c13)c1cc2ccccc2c2c3c4ccccc4cc(-c4ccc6c(c4)c4ccccc4n6-c4ccccc4)c3n5c12 ; CC(C)(C)c1cc(N(c2ccccc2)c2ccccc2)c2c(c1)c1cc(C(C)(C)C)cc3c4cc5c(nc4n2c31)c1cc2ccccc2c2c3c4ccccc4ccc3n5c12]

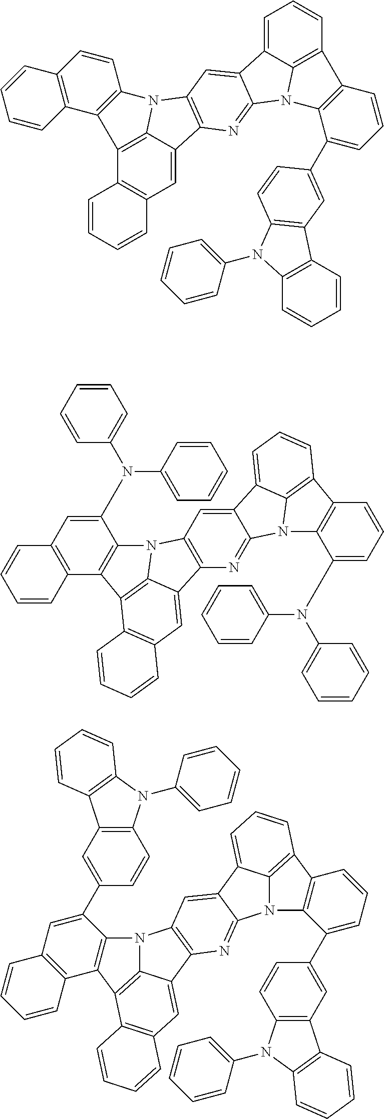 [SMILES: c1ccc(-n2c3ccccc3c3cc(-c4cccc5c6cccc7c8cc9c(nc8n(c45)c76)c4cc5ccccc5c5c6c7ccccc7ccc6n9c45)ccc32)cc1 ; c1ccc(N(c2ccccc2)c2cc3ccccc3c3c4c5ccccc5cc5c6nc7c(cc6n(c23)c54)c2cccc3c4cccc(N(c5ccccc5)c5ccccc5)c4n7c23)cc1 ; c1ccc(-n2c3ccccc3c3cc(-c4cc5ccccc5c5c6c7ccccc7cc7c8nc9c(cc8n(c45)c76)c4cccc5c6cccc(-c7ccc8c(c7)c7ccccc7n8-c7ccccc7)c6n9c45)ccc32)cc1]

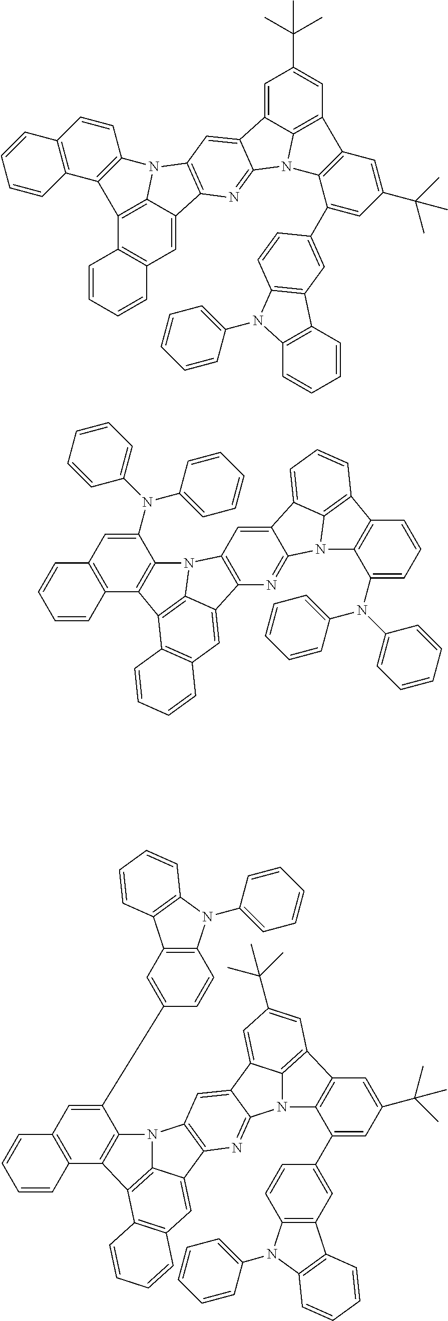 [SMILES: CC(C)(C)c1cc(-c2ccc3c(c2)c2ccccc2n3-c2ccccc2)c2c(c1)c1cc(C(C)(C)C)cc3c4cc5c(nc4n2c31)c1cc2ccccc2c2c3c4ccccc4ccc3n5c12 ; c1ccc(N(c2ccccc2)c2cc3ccccc3c3c4c5ccccc5cc5c6nc7c(cc6n(c23)c54)c2cccc3c4cccc(N(c5ccccc5)c5ccccc5)c4n7c23)cc1 ; CC(C)(C)c1cc(-c2ccc3c(c2)c2ccccc2n3-c2ccccc2)c2c(c1)c1cc(C(C)(C)C)cc3c4cc5c(nc4n2c31)c1cc2ccccc2c2c3c4ccccc4cc(-c4ccc6c(c4)c4ccccc4n6-c4ccccc4)c3n5c12]

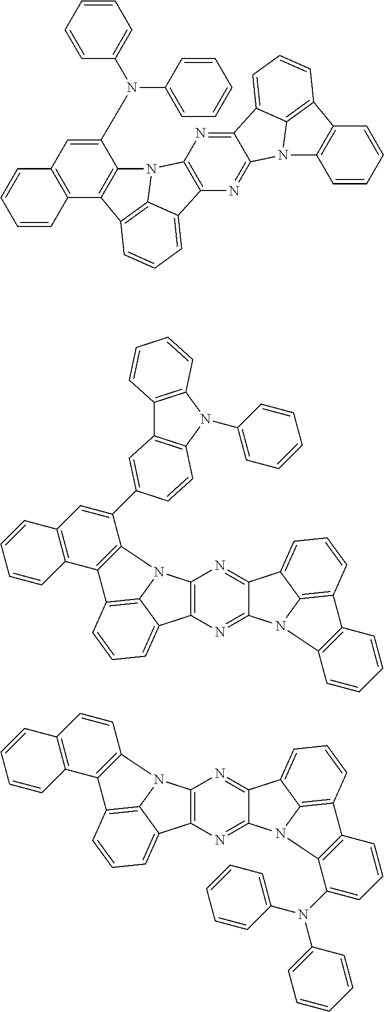 [SMILES: c1ccc(N(c2ccccc2)c2cc3ccccc3c3c4cccc5c6nc7c(nc6n(c23)c54)c2cccc3c4ccccc4n7c32)cc1 ; c1ccc(-n2c3ccccc3c3cc(-c4cc5ccccc5c5c6cccc7c8nc9c(nc8n(c45)c76)c4cccc5c6ccccc6n9c54)ccc32)cc1 ; c1ccc(N(c2ccccc2)c2cccc3c4cccc5c6nc7c(nc6n(c23)c45)c2cccc3c4c5ccccc5ccc4n7c23)cc1]

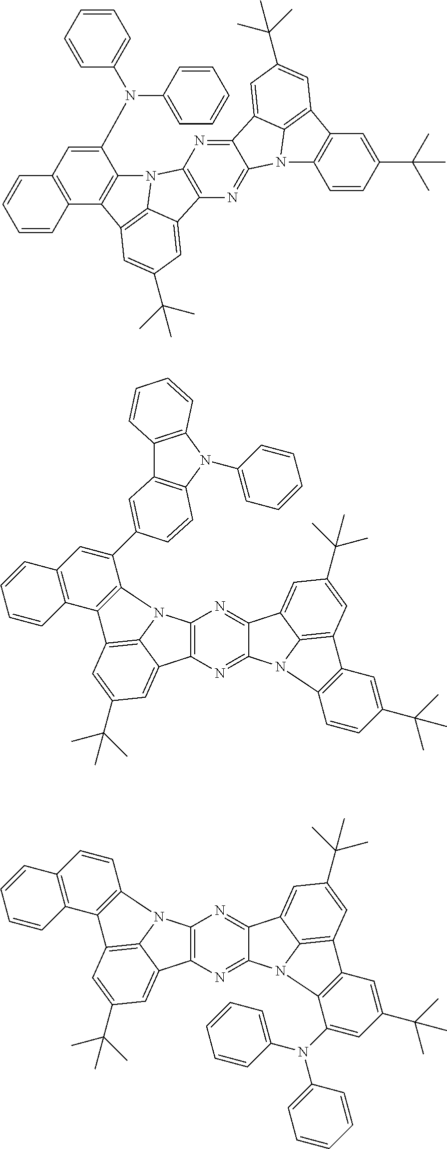

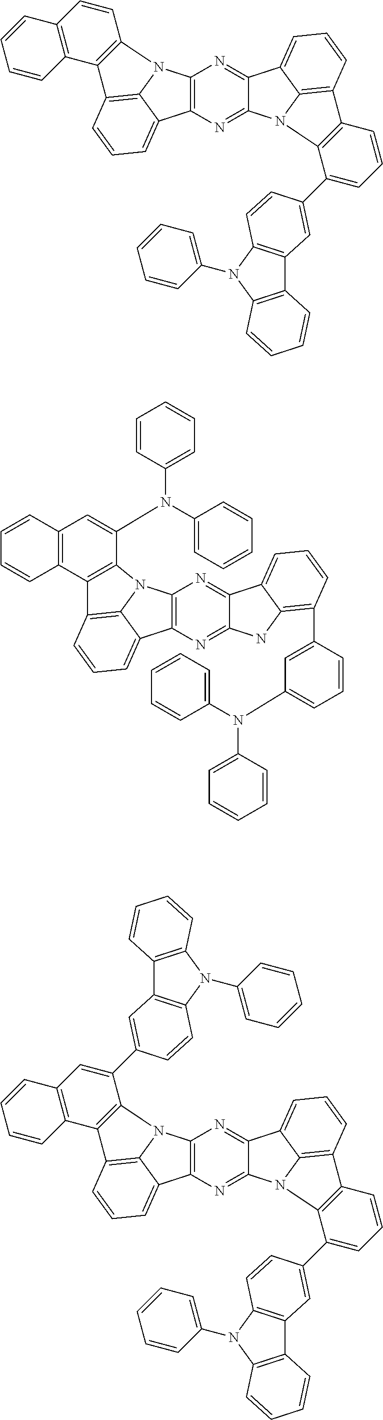 [SMILES: c1ccc(-n2c3ccccc3c3cc(-c4cccc5c6cccc7c8nc9c(nc8n(c45)c67)c4cccc5c6c7ccccc7ccc6n9c45)ccc32)cc1 ; c1ccc(N(c2ccccc2)c2cccc(-c3cccc4c3[nH]c3nc5c6cccc7c8c9ccccc9cc(N(c9ccccc9)c9ccccc9)c8n(c5nc34)c67)c2)cc1 ; c1ccc(-n2c3ccccc3c3cc(-c4cccc5c6cccc7c8nc9c(nc8n(c45)c67)c4cccc5c6c7ccccc7cc(-c7ccc8c(c7)c7ccccc7n8-c7ccccc7)c6n9c45)ccc32)cc1]

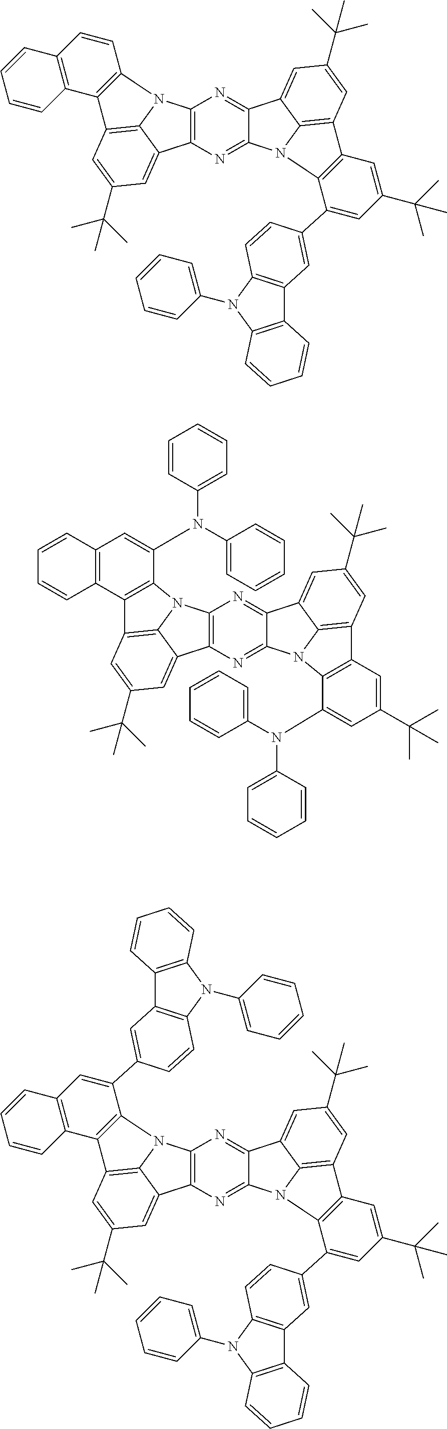 [SMILES: CC(C)(C)c1cc(-c2ccc3c(c2)c2ccccc2n3-c2ccccc2)c2c(c1)c1cc(C(C)(C)C)cc3c4nc5c(nc4n2c13)c1cc(C(C)(C)C)cc2c3c4ccccc4ccc3n5c12 ; CC(C)(C)c1cc(N(c2ccccc2)c2ccccc2)c2c(c1)c1cc(C(C)(C)C)cc3c4nc5c(nc4n2c13)c1cc(C(C)(C)C)cc2c3c4ccccc4cc(N(c4ccccc4)c4ccccc4)c3n5c12 ; CC(C)(C)c1cc(-c2ccc3c(c2)c2ccccc2n3-c2ccccc2)c2c(c1)c1cc(C(C)(C)C)cc3c4nc5c(nc4n2c13)c1cc(C(C)(C)C)cc2c3c4ccccc4cc(-c4ccc6c(c4)c4ccccc4n6-c4ccccc4)c3n5c12]

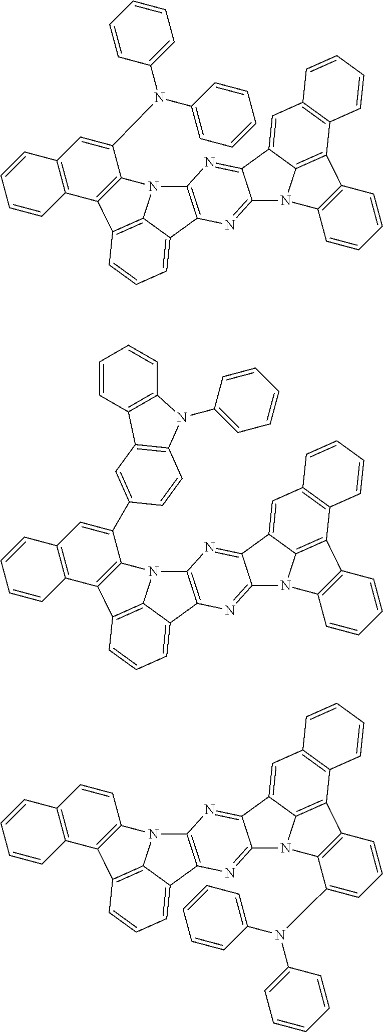 [SMILES: c1ccc(N(c2ccccc2)c2cc3ccccc3c3c4cccc5c6nc7c(nc6n(c23)c54)c2cc3ccccc3c3c4ccccc4n7c23)cc1 ; c1ccc(-n2c3ccccc3c3cc(-c4cc5ccccc5c5c6cccc7c8nc9c(nc8n(c45)c76)c4cc5ccccc5c5c6ccccc6n9c45)ccc32)cc1 ; c1ccc(N(c2ccccc2)c2cccc3c4c5ccccc5cc5c6nc7c(nc6n(c23)c54)c2cccc3c4c5ccccc5ccc4n7c23)cc1]

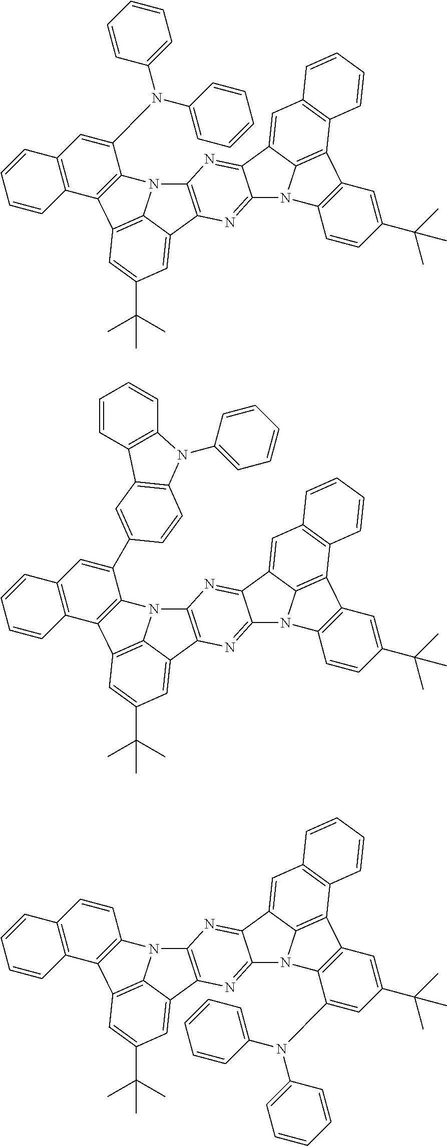

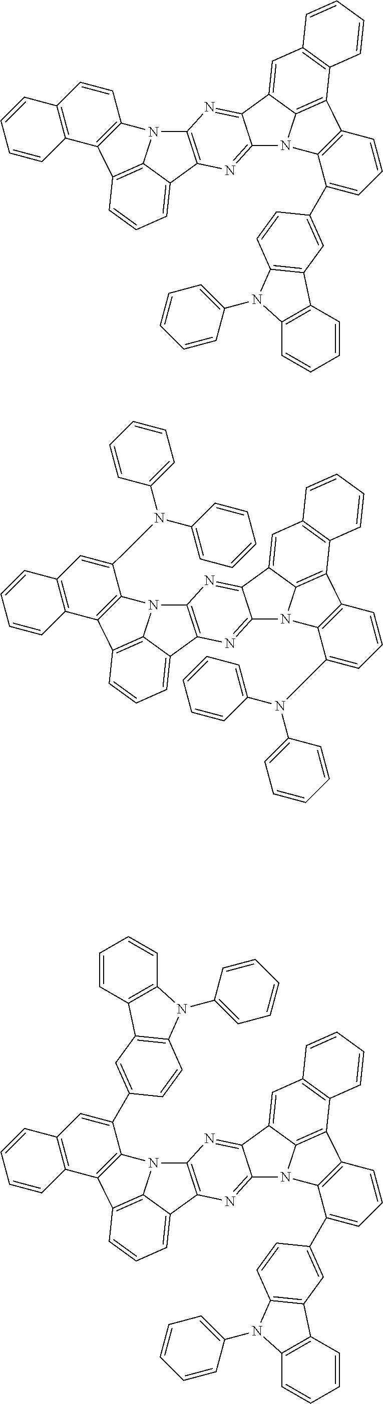 [SMILES: c1ccc(-n2c3ccccc3c3cc(-c4cccc5c6c7ccccc7cc7c8nc9c(nc8n(c45)c76)c4cccc5c6c7ccccc7ccc6n9c45)ccc32)cc1 ; c1ccc(N(c2ccccc2)c2cc3ccccc3c3c4cccc5c6nc7c(nc6n(c23)c54)c2cc3ccccc3c3c4cccc(N(c5ccccc5)c5ccccc5)c4n7c23)cc1 ; c1ccc(-n2c3ccccc3c3cc(-c4cc5ccccc5c5c6cccc7c8nc9c(nc8n(c45)c76)c4cc5ccccc5c5c6cccc(-c7ccc8c(c7)c7ccccc7n8-c7ccccc7)c6n9c45)ccc32)cc1]

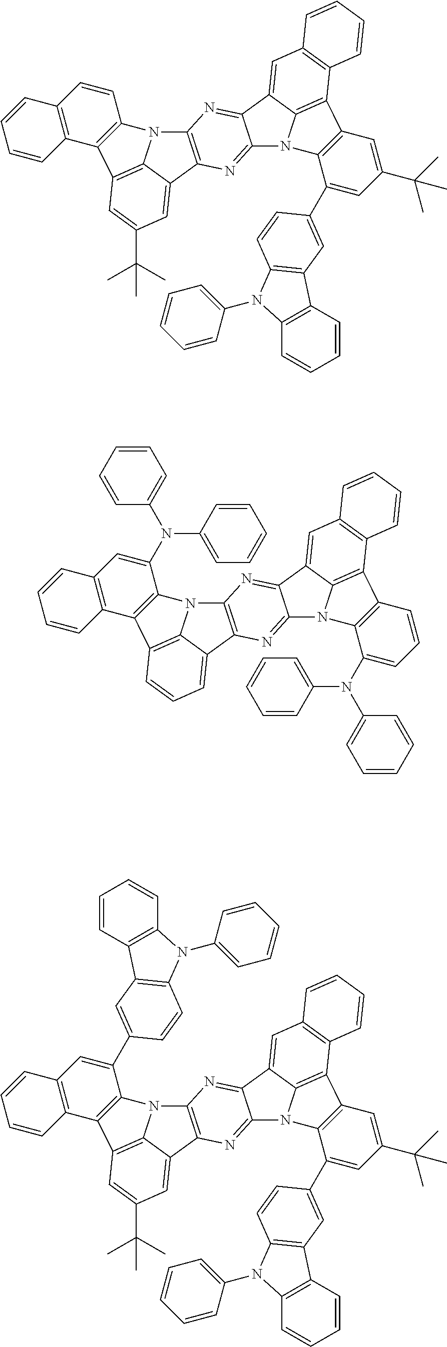 [SMILES: CC(C)(C)c1cc(-c2ccc3c(c2)c2ccccc2n3-c2ccccc2)c2c(c1)c1c3ccccc3cc3c4nc5c(nc4n2c31)c1cc(C(C)(C)C)cc2c3c4ccccc4ccc3n5c12 ; c1ccc(N(c2ccccc2)c2cc3ccccc3c3c4cccc5c6nc7c(nc6n(c23)c54)c2cc3ccccc3c3c4cccc(N(c5ccccc5)c5ccccc5)c4n7c23)cc1 ; CC(C)(C)c1cc(-c2ccc3c(c2)c2ccccc2n3-c2ccccc2)c2c(c1)c1c3ccccc3cc3c4nc5c(nc4n2c31)c1cc(C(C)(C)C)cc2c3c4ccccc4cc(-c4ccc6c(c4)c4ccccc4n6-c4ccccc4)c3n5c12]

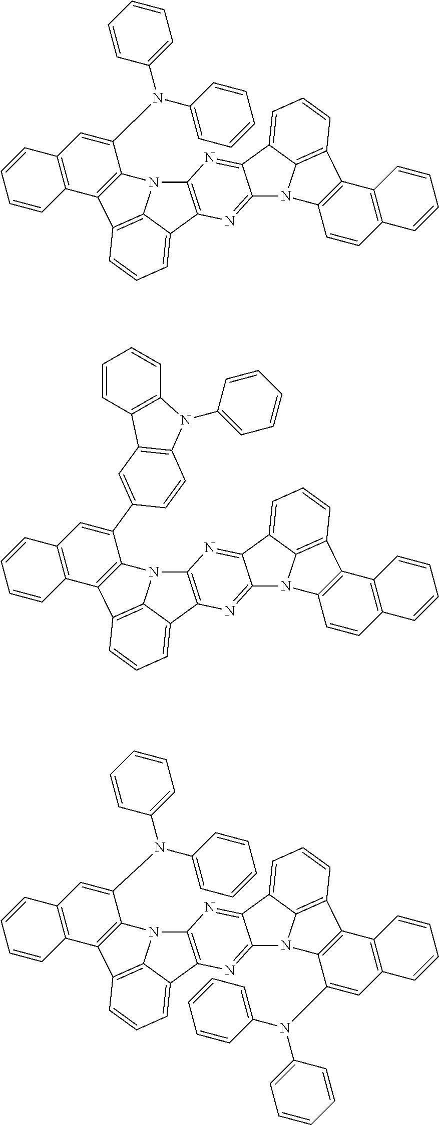 [SMILES: c1ccc(N(c2ccccc2)c2cc3ccccc3c3c4cccc5c6nc7c(nc6n(c23)c54)c2cccc3c4c5ccccc5ccc4n7c23)cc1 ; c1ccc(-n2c3ccccc3c3cc(-c4cc5ccccc5c5c6cccc7c8nc9c(nc8n(c45)c76)c4cccc5c6c7ccccc7ccc6n9c45)ccc32)cc1 ; c1ccc(N(c2ccccc2)c2cc3ccccc3c3c4cccc5c6nc7c(nc6n(c23)c54)c2cccc3c4c5ccccc5cc(N(c5ccccc5)c5ccccc5)c4n7c23)cc1]

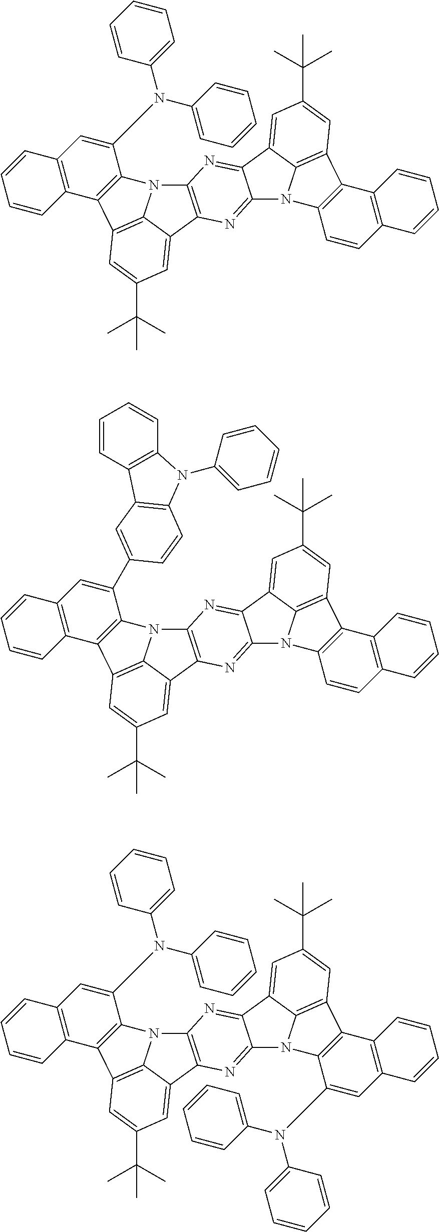

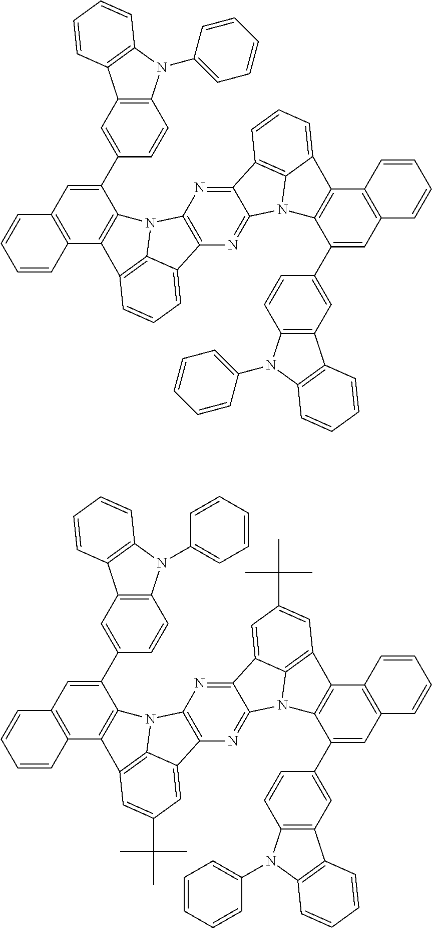 [SMILES: c1ccc(-n2c3ccccc3c3cc(-c4cc5ccccc5c5c6cccc7c8nc9c(nc8n(c45)c76)c4cccc5c6c7ccccc7cc(-c7ccc8c(c7)c7ccccc7n8-c7ccccc7)c6n9c45)ccc32)cc1 ; CC(C)(C)c1cc2c3nc4c(nc3n3c5c(-c6ccc7c(c6)c6ccccc6n7-c6ccccc6)cc6ccccc6c5c(c1)c23)c1cc(C(C)(C)C)cc2c3c5ccccc5cc(-c5ccc6c(c5)c5ccccc5n6-c5ccccc5)c3n4c12]

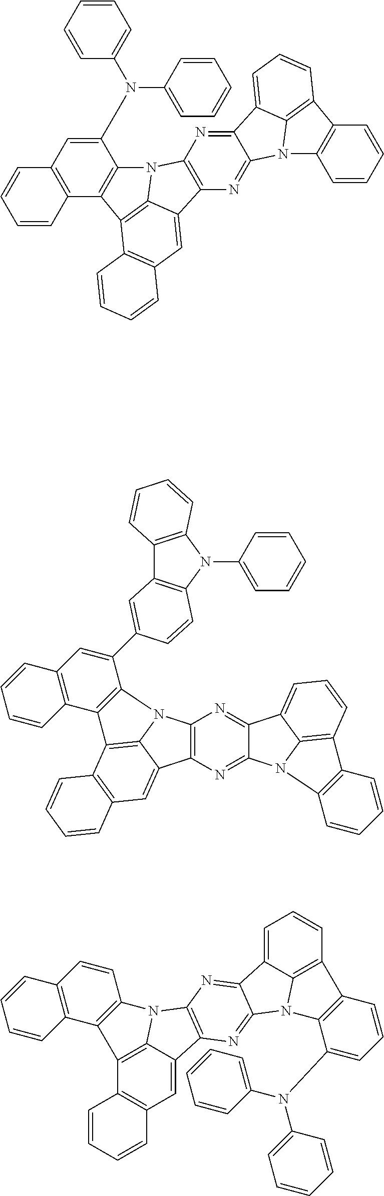 [SMILES: c1ccc(N(c2ccccc2)c2cc3ccccc3c3c4c5ccccc5cc5c6nc7c(nc6n(c23)c54)c2cccc3c4ccccc4n7c32)cc1 ; c1ccc(-n2c3ccccc3c3cc(-c4cc5ccccc5c5c6c7ccccc7cc7c8nc9c(nc8n(c45)c76)c4cccc5c6ccccc6n9c54)ccc32)cc1 ; c1ccc(N(c2ccccc2)c2cccc3c4cccc5c6nc7c(nc6n(c23)c45)c2cc3ccccc3c3c4c5ccccc5ccc4n7c23)cc1]

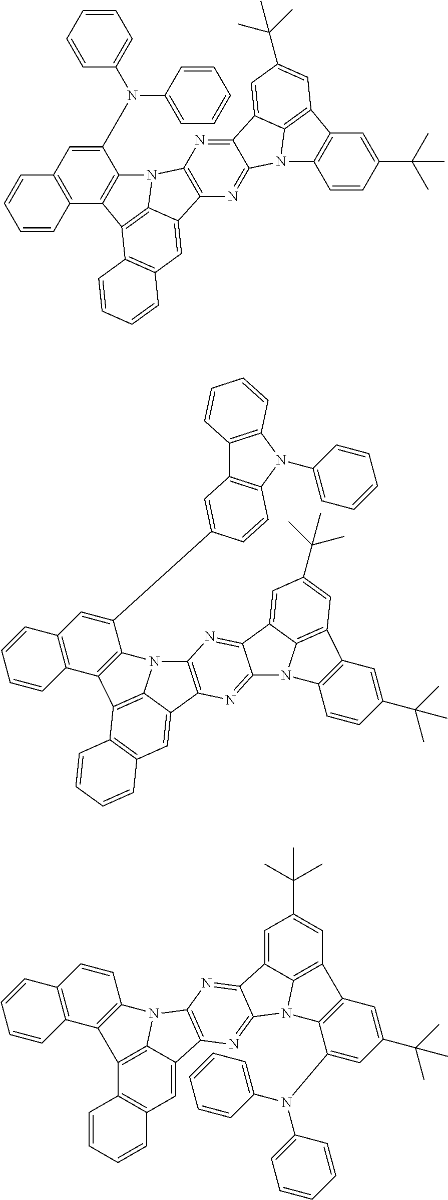 [SMILES: CC(C)(C)c1ccc2c(c1)c1cc(C(C)(C)C)cc3c4nc5c(nc4n2c13)c1cc2ccccc2c2c3c4ccccc4cc(N(c4ccccc4)c4ccccc4)c3n5c12 ; CC(C)(C)c1ccc2c(c1)c1cc(C(C)(C)C)cc3c4nc5c(nc4n2c13)c1cc2ccccc2c2c3c4ccccc4cc(-c4ccc6c(c4)c4ccccc4n6-c4ccccc4)c3n5c12 ; CC(C)(C)c1cc(N(c2ccccc2)c2ccccc2)c2c(c1)c1cc(C(C)(C)C)cc3c4nc5c(nc4n2c13)c1cc2ccccc2c2c3c4ccccc4ccc3n5c12]

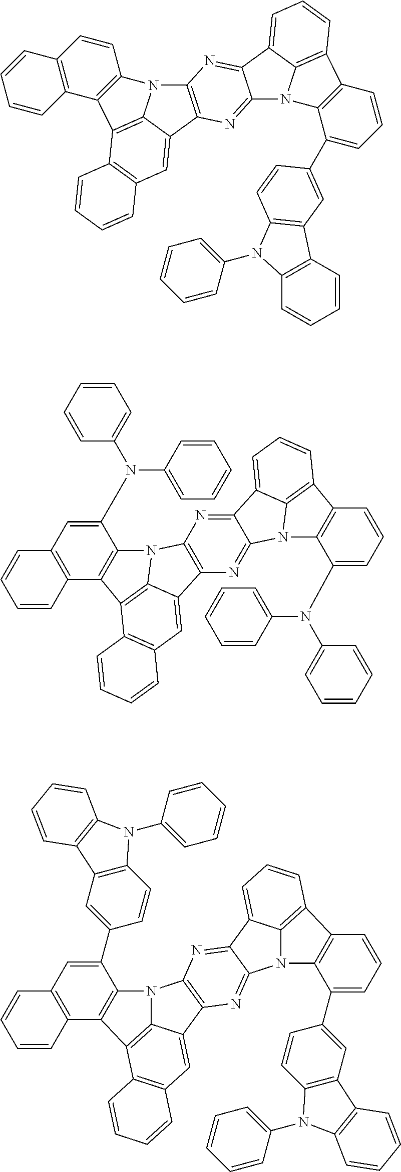 [SMILES: c1ccc(-n2c3ccccc3c3cc(-c4cccc5c6cccc7c8nc9c(nc8n(c45)c67)c4cc5ccccc5c5c6c7ccccc7ccc6n9c45)ccc32)cc1 ; c1ccc(N(c2ccccc2)c2cccc3c4cccc5c6nc7c(nc6n(c23)c45)c2cc3ccccc3c3c4c5ccccc5cc(N(c5ccccc5)c5ccccc5)c4n7c23)cc1 ; c1ccc(-n2c3ccccc3c3cc(-c4cccc5c6cccc7c8nc9c(nc8n(c45)c67)c4cc5ccccc5c5c6c7ccccc7cc(-c7ccc8c(c7)c7ccccc7n8-c7ccccc7)c6n9c45)ccc32)cc1]

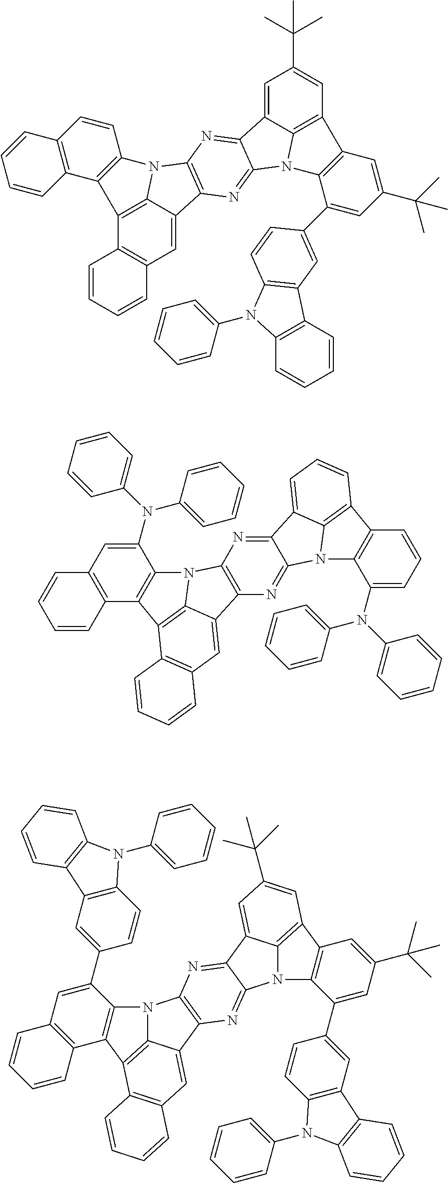 [SMILES: CC(C)(C)c1cc(-c2ccc3c(c2)c2ccccc2n3-c2ccccc2)c2c(c1)c1cc(C(C)(C)C)cc3c4nc5c(nc4n2c13)c1cc2ccccc2c2c3c4ccccc4ccc3n5c12 ; c1ccc(N(c2ccccc2)c2cccc3c4cccc5c6nc7c(nc6n(c23)c45)c2cc3ccccc3c3c4c5ccccc5cc(N(c5ccccc5)c5ccccc5)c4n7c23)cc1 ; CC(C)(C)c1cc(-c2ccc3c(c2)c2ccccc2n3-c2ccccc2)c2c(c1)c1cc(C(C)(C)C)cc3c4nc5c(nc4n2c13)c1cc2ccccc2c2c3c4ccccc4cc(-c4ccc6c(c4)c4ccccc4n6-c4ccccc4)c3n5c12]

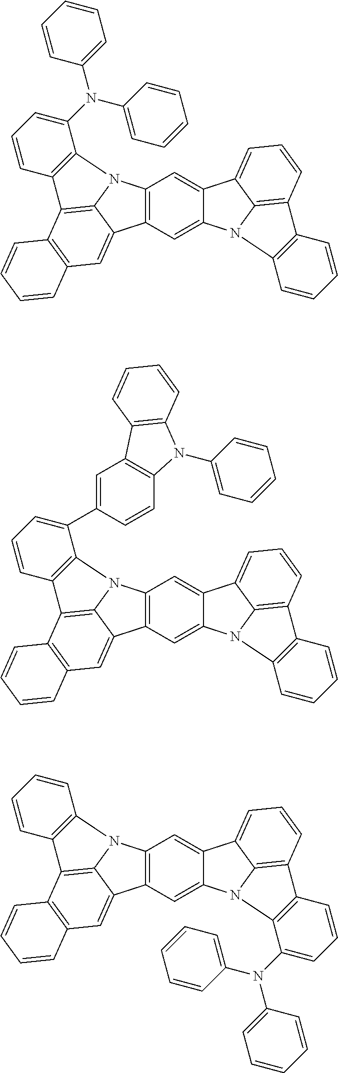 [SMILES: c1ccc(N(c2ccccc2)c2cccc3c4c5ccccc5cc5c6cc7c(cc6n(c23)c54)c2cccc3c4ccccc4n7c32)cc1 ; c1ccc(-n2c3ccccc3c3cc(-c4cccc5c6c7ccccc7cc7c8cc9c(cc8n(c45)c76)c4cccc5c6ccccc6n9c54)ccc32)cc1 ; c1ccc(N(c2ccccc2)c2cccc3c4cccc5c6cc7c(cc6n(c23)c54)c2cc3ccccc3c3c4ccccc4n7c23)cc1]

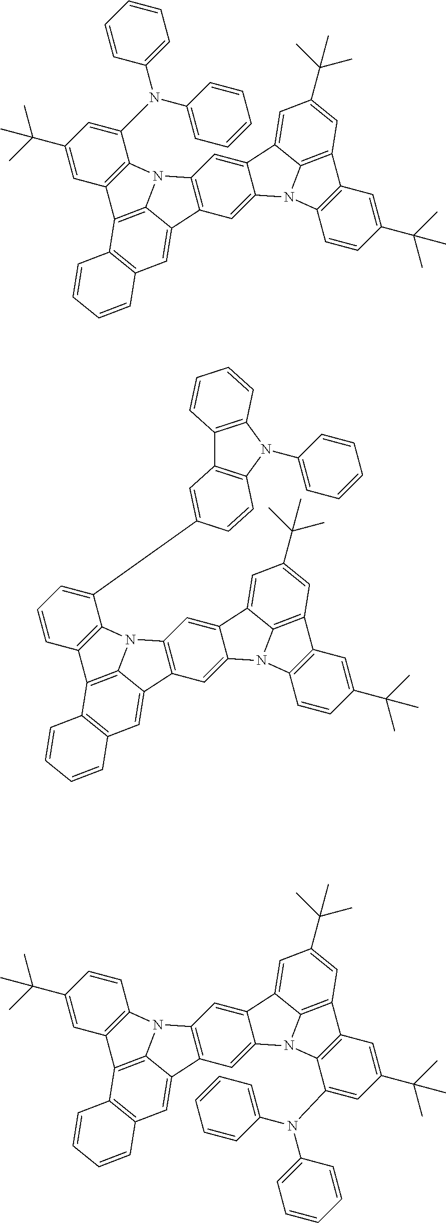

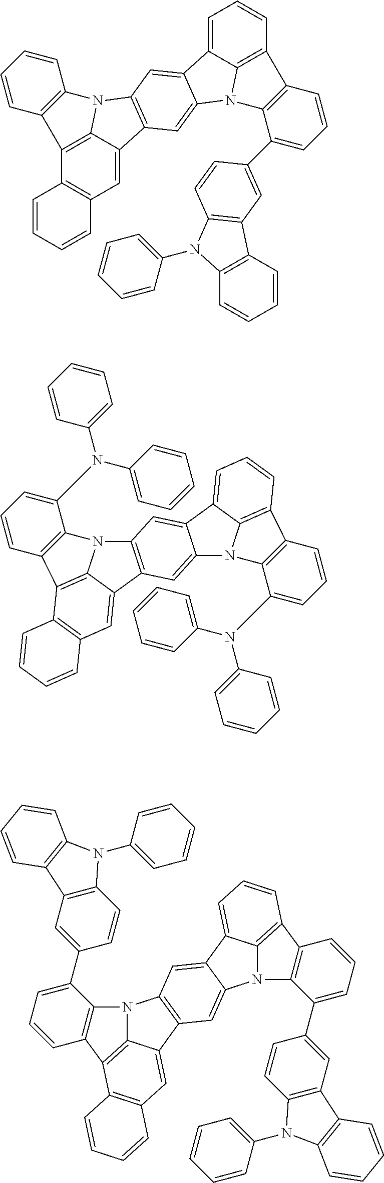 [SMILES: c1ccc(-n2c3ccccc3c3cc(-c4cccc5c6cccc7c8cc9c(cc8n(c45)c76)c4cc5ccccc5c5c6ccccc6n9c45)ccc32)cc1 ; c1ccc(N(c2ccccc2)c2cccc3c4cccc5c6cc7c(cc6n(c23)c54)c2cc3ccccc3c3c4cccc(N(c5ccccc5)c5ccccc5)c4n7c23)cc1 ; c1ccc(-n2c3ccccc3c3cc(-c4cccc5c6cccc7c8cc9c(cc8n(c45)c76)c4cc5ccccc5c5c6cccc(-c7ccc8c(c7)c7ccccc7n8-c7ccccc7)c6n9c45)ccc32)cc1]

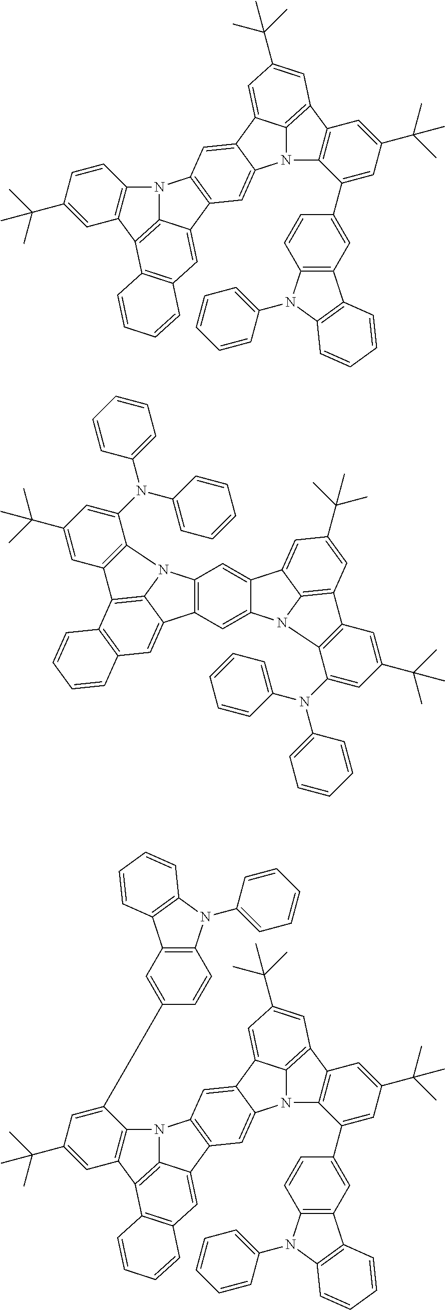

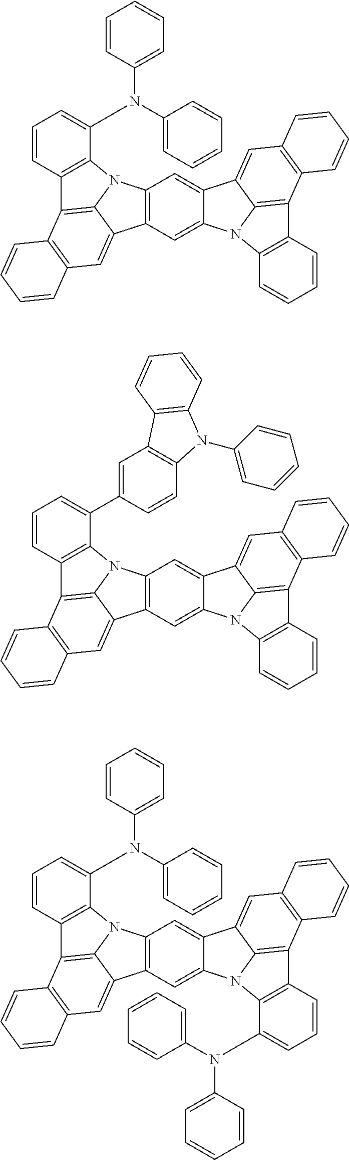 [SMILES: c1ccc(N(c2ccccc2)c2cccc3c4c5ccccc5cc5c6cc7c(cc6n(c23)c54)c2cc3ccccc3c3c4ccccc4n7c23)cc1 ; c1ccc(-n2c3ccccc3c3cc(-c4cccc5c6c7ccccc7cc7c8cc9c(cc8n(c45)c76)c4cc5ccccc5c5c6ccccc6n9c45)ccc32)cc1 ; c1ccc(N(c2ccccc2)c2cccc3c4c5ccccc5cc5c6cc7c(cc6n(c23)c54)c2cc3ccccc3c3c4cccc(N(c5ccccc5)c5ccccc5)c4n7c23)cc1]

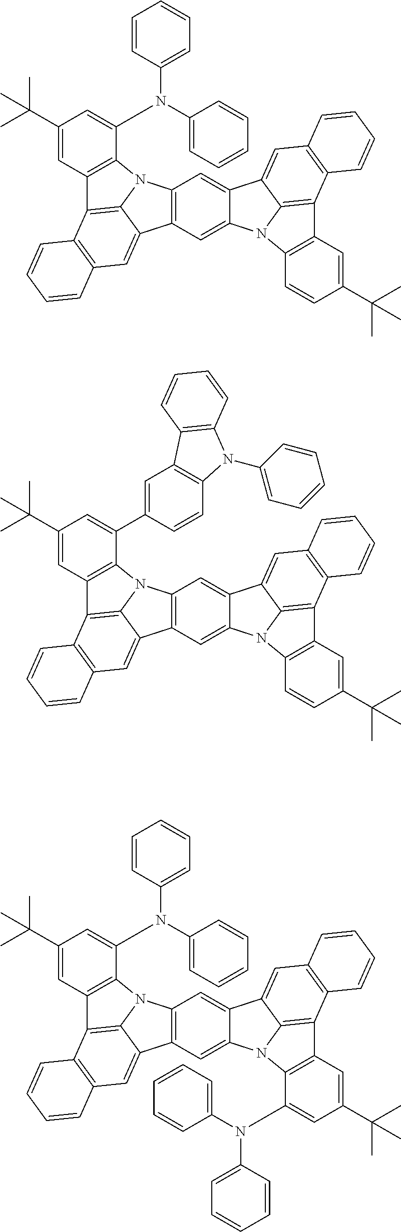

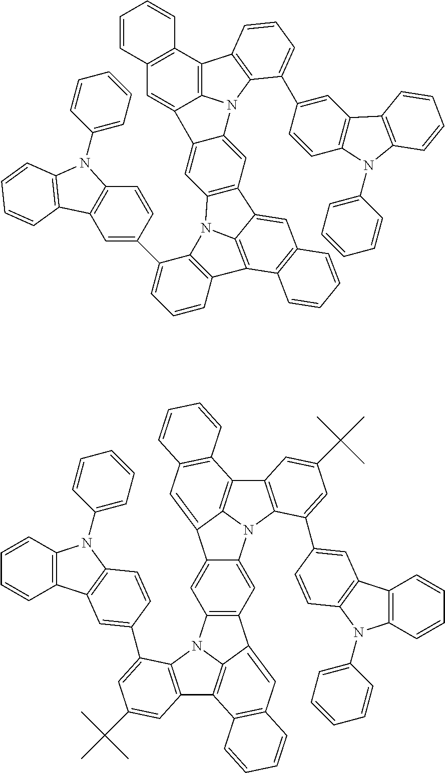 [SMILES: c1ccc(-n2c3ccccc3c3cc(-c4cccc5c6c7ccccc7cc7c8cc9c(cc8n(c45)c76)c4cc5ccccc5c5c6cccc(-c7ccc8c(c7)c7ccccc7n8-c7ccccc7)c6n9c45)ccc32)cc1 ; CC(C)(C)c1cc(-c2ccc3c(c2)c2ccccc2n3-c2ccccc2)c2c(c1)c1c3ccccc3cc3c4cc5c(cc4n2c31)c1cc2ccccc2c2c3cc(C(C)(C)C)cc(-c4ccc6c(c4)c4ccccc4n6-c4ccccc4)c3n5c12]

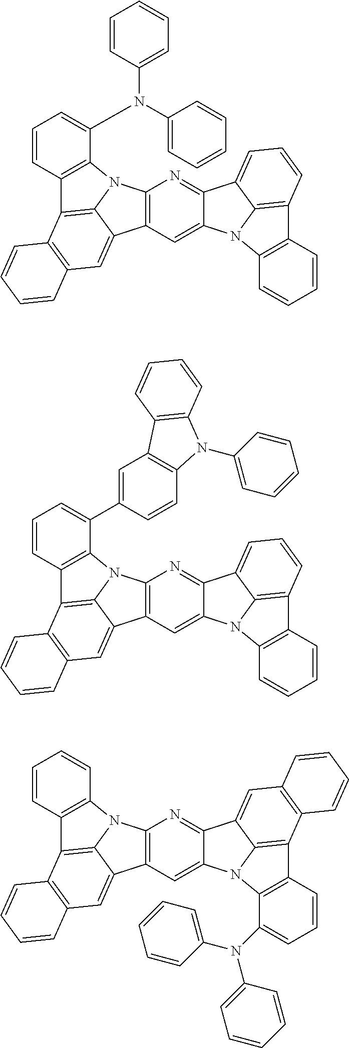 [SMILES: c1ccc(N(c2ccccc2)c2cccc3c4c5ccccc5cc5c6cc7c(nc6n(c23)c54)c2cccc3c4ccccc4n7c32)cc1 ; c1ccc(-n2c3ccccc3c3cc(-c4cccc5c6c7ccccc7cc7c8cc9c(nc8n(c45)c76)c4cccc5c6ccccc6n9c54)ccc32)cc1 ; c1ccc(N(c2ccccc2)c2cccc3c4c5ccccc5cc5c6nc7c(cc6n(c23)c54)c2cc3ccccc3c3c4ccccc4n7c23)cc1]

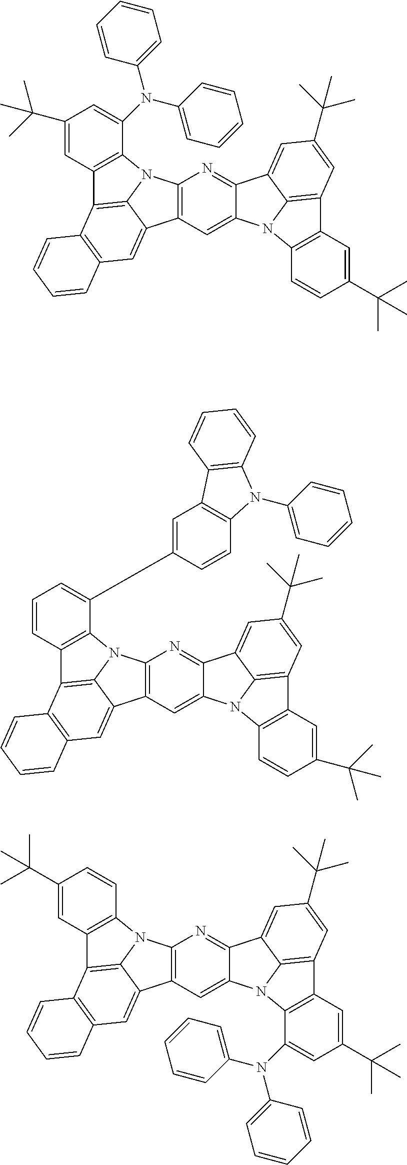

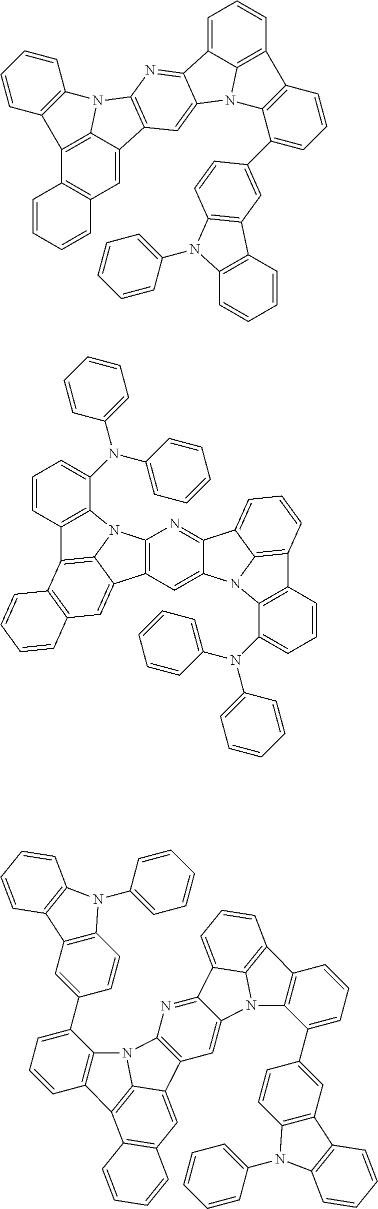 [SMILES: c1ccc(-n2c3ccccc3c3cc(-c4cccc5c6cccc7c8nc9c(cc8n(c45)c67)c4cc5ccccc5c5c6ccccc6n9c45)ccc32)cc1 ; c1ccc(N(c2ccccc2)c2cccc3c4cccc5c6nc7c(cc6n(c23)c45)c2cc3ccccc3c3c4cccc(N(c5ccccc5)c5ccccc5)c4n7c23)cc1 ; c1ccc(-n2c3ccccc3c3cc(-c4cccc5c6cccc7c8nc9c(cc8n(c45)c67)c4cc5ccccc5c5c6cccc(-c7ccc8c(c7)c7ccccc7n8-c7ccccc7)c6n9c45)ccc32)cc1]

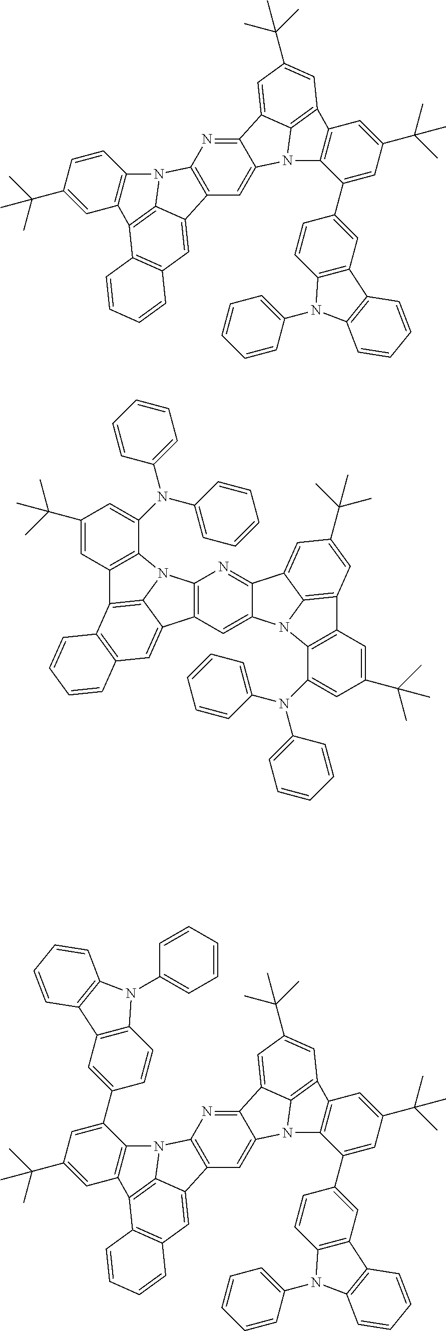

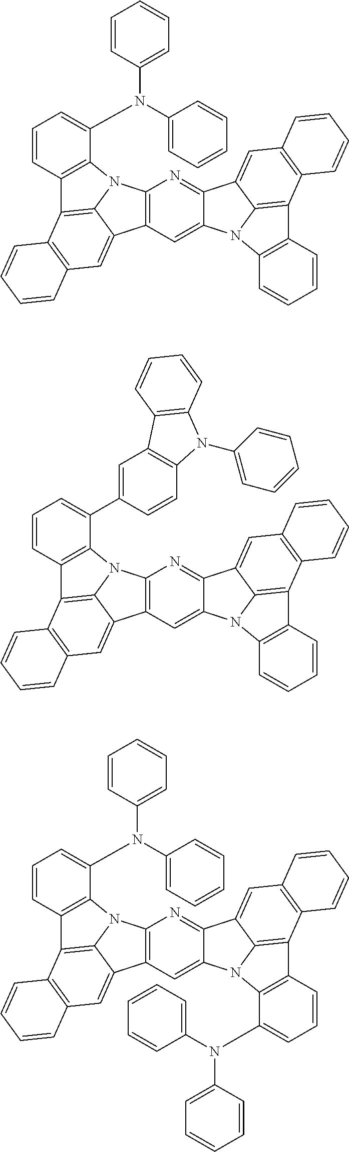 [SMILES: c1ccc(N(c2ccccc2)c2cccc3c4c5ccccc5cc5c6cc7c(nc6n(c23)c54)c2cc3ccccc3c3c4ccccc4n7c23)cc1 ; c1ccc(-n2c3ccccc3c3cc(-c4cccc5c6c7ccccc7cc7c8cc9c(nc8n(c45)c76)c4cc5ccccc5c5c6ccccc6n9c45)ccc32)cc1 ; c1ccc(N(c2ccccc2)c2cccc3c4c5ccccc5cc5c6nc7c(cc6n(c23)c54)c2cc3ccccc3c3c4cccc(N(c5ccccc5)c5ccccc5)c4n7c23)cc1]

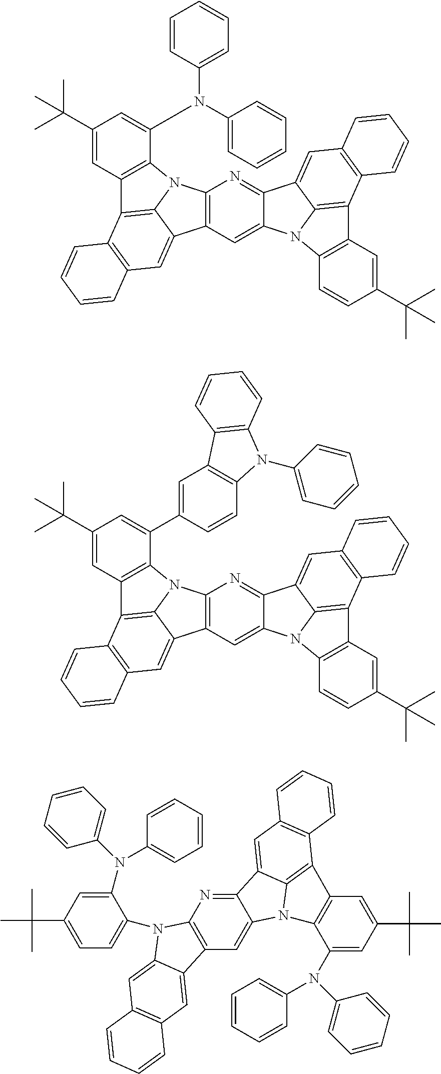

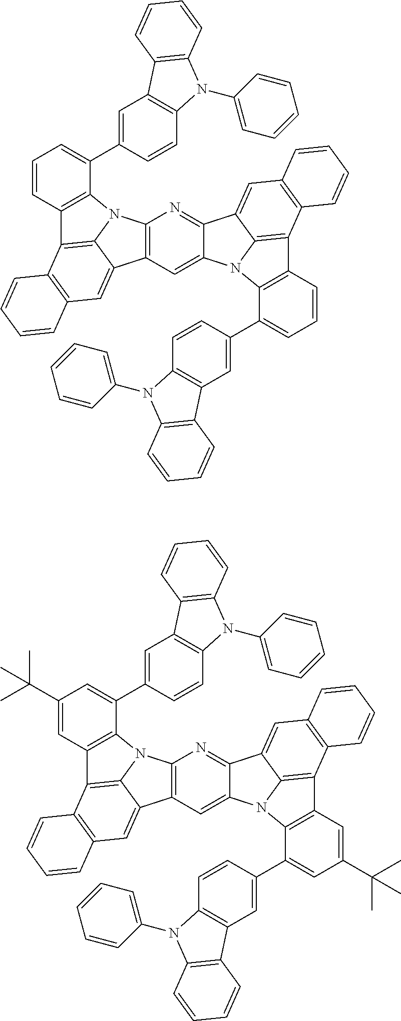 [SMILES: c1ccc(-n2c3ccccc3c3cc(-c4cccc5c6c7ccccc7cc7c8nc9c(cc8n(c45)c76)c4cc5ccccc5c5c6cccc(-c7ccc8c(c7)c7ccccc7n8-c7ccccc7)c6n9c45)ccc32)cc1 ; CC(C)(C)c1cc(-c2ccc3c(c2)c2ccccc2n3-c2ccccc2)c2c(c1)c1c3ccccc3cc3c4nc5c(cc4n2c31)c1cc2ccccc2c2c3cc(C(C)(C)C)cc(-c4ccc6c(c4)c4ccccc4n6-c4ccccc4)c3n5c12]

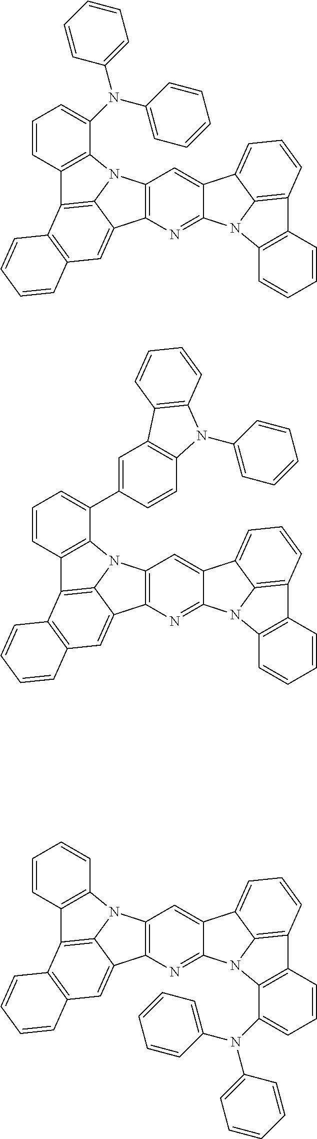 [SMILES: c1ccc(N(c2ccccc2)c2cccc3c4c5ccccc5cc5c6nc7c(cc6n(c23)c54)c2cccc3c4ccccc4n7c32)cc1 ; c1ccc(-n2c3ccccc3c3cc(-c4cccc5c6c7ccccc7cc7c8nc9c(cc8n(c45)c76)c4cccc5c6ccccc6n9c54)ccc32)cc1 ; c1ccc(N(c2ccccc2)c2cccc3c4cccc5c6cc7c(nc6n(c23)c54)c2cc3ccccc3c3c4ccccc4n7c23)cc1]

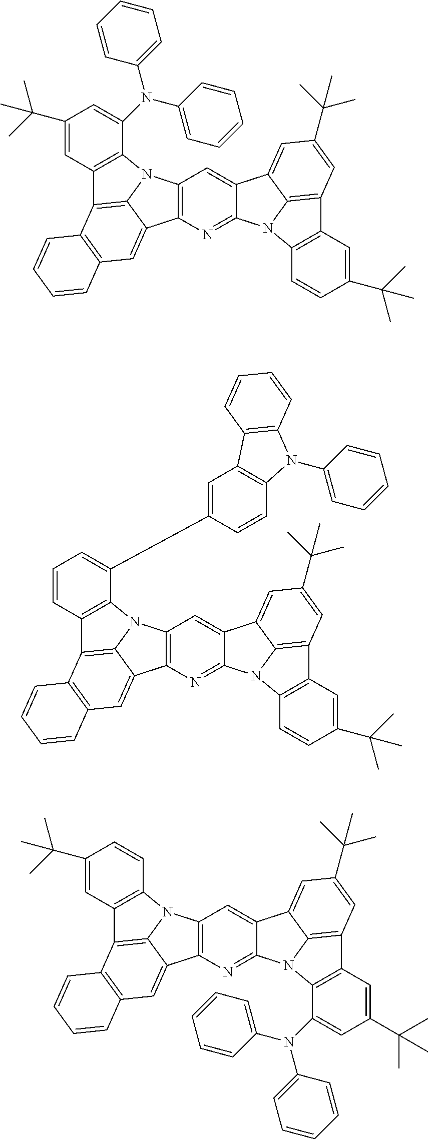

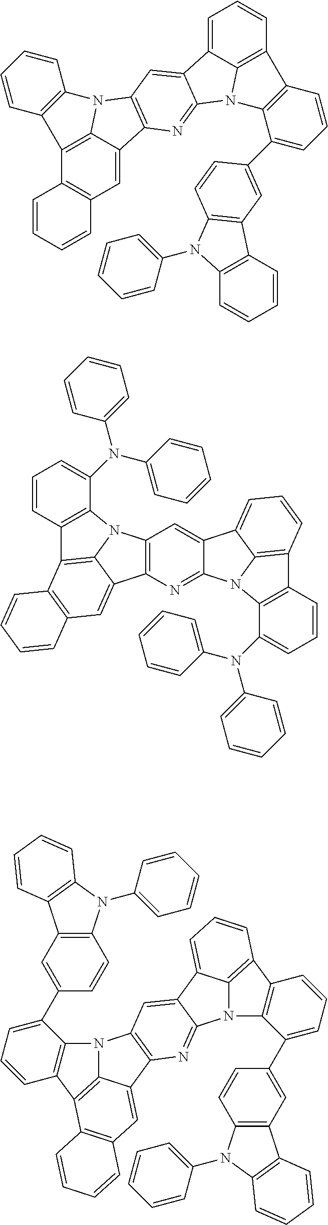 [SMILES: c1ccc(-n2c3ccccc3c3cc(-c4cccc5c6cccc7c8cc9c(nc8n(c45)c76)c4cc5ccccc5c5c6ccccc6n9c45)ccc32)cc1 ; c1ccc(N(c2ccccc2)c2cccc3c4c5ccccc5cc5c6nc7c(cc6n(c23)c54)c2cccc3c4cccc(N(c5ccccc5)c5ccccc5)c4n7c23)cc1 ; c1ccc(-n2c3ccccc3c3cc(-c4cccc5c6c7ccccc7cc7c8nc9c(cc8n(c45)c76)c4cccc5c6cccc(-c7ccc8c(c7)c7ccccc7n8-c7ccccc7)c6n9c45)ccc32)cc1]

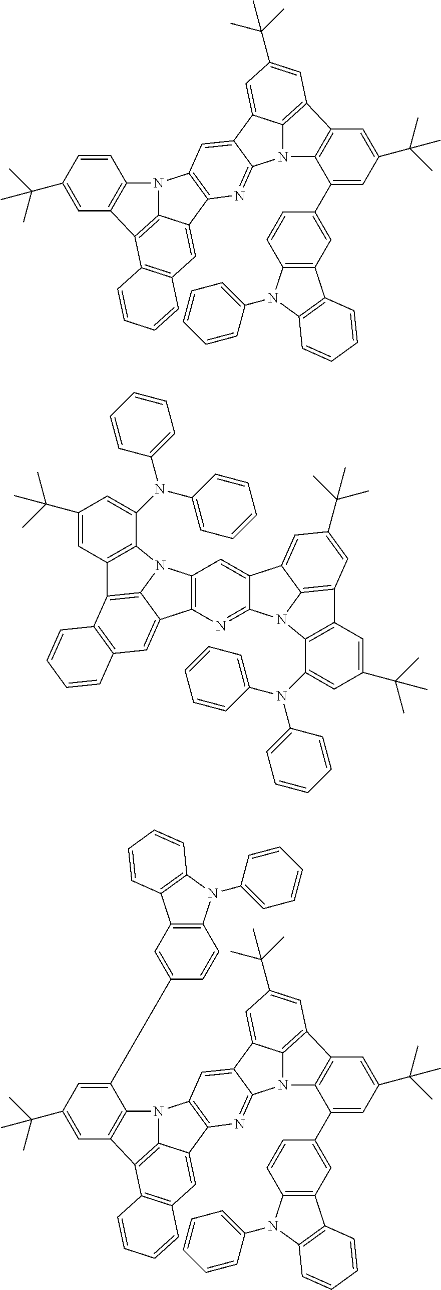

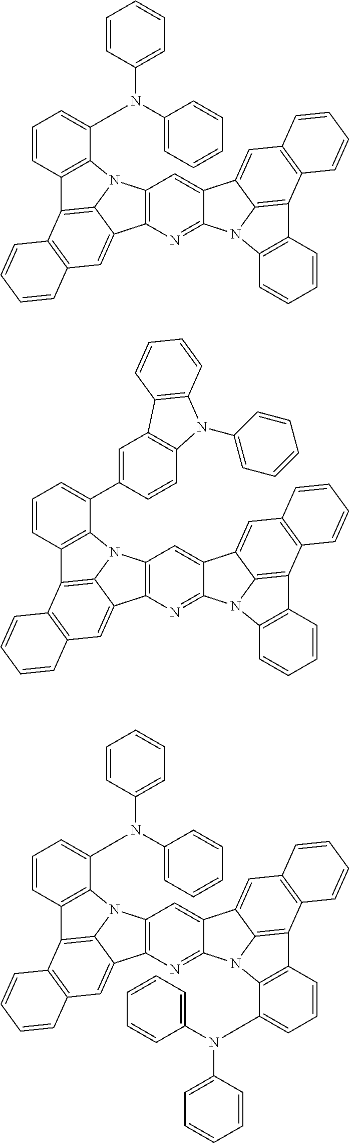 [SMILES: c1ccc(N(c2ccccc2)c2cccc3c4c5ccccc5cc5c6nc7c(cc6n(c23)c54)c2cc3ccccc3c3c4ccccc4n7c23)cc1 ; c1ccc(-n2c3ccccc3c3cc(-c4cccc5c6c7ccccc7cc7c8nc9c(cc8n(c45)c76)c4cc5ccccc5c5c6ccccc6n9c45)ccc32)cc1 ; c1ccc(N(c2ccccc2)c2cccc3c4c5ccccc5cc5c6nc7c(cc6n(c23)c54)c2cc3ccccc3c3c4cccc(N(c5ccccc5)c5ccccc5)c4n7c23)cc1]

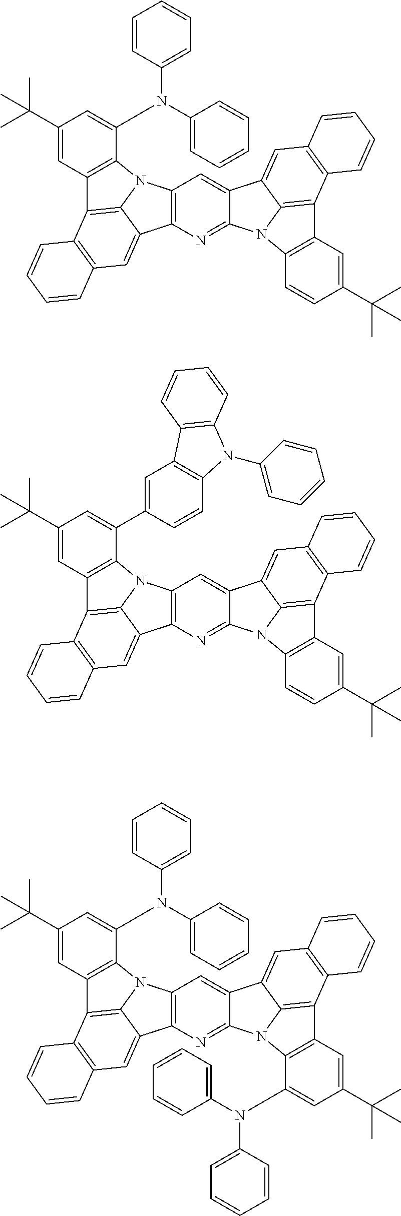

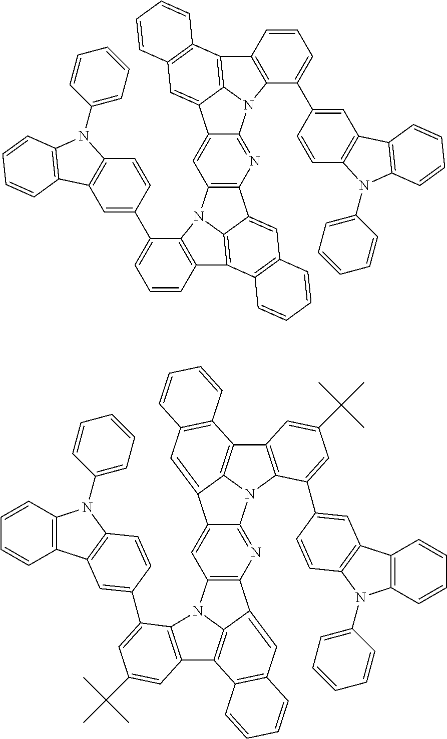 [SMILES: c1ccc(-n2c3ccccc3c3cc(-c4cccc5c6c7ccccc7cc7c8nc9c(cc8n(c45)c76)c4cc5ccccc5c5c6cccc(-c7ccc8c(c7)c7ccccc7n8-c7ccccc7)c6n9c45)ccc32)cc1 ; CC(C)(C)c1cc(-c2ccc3c(c2)c2ccccc2n3-c2ccccc2)c2c(c1)c1c3ccccc3cc3c4nc5c(cc4n2c31)c1cc2ccccc2c2c3cc(C(C)(C)C)cc(-c4ccc6c(c4)c4ccccc4n6-c4ccccc4)c3n5c12]

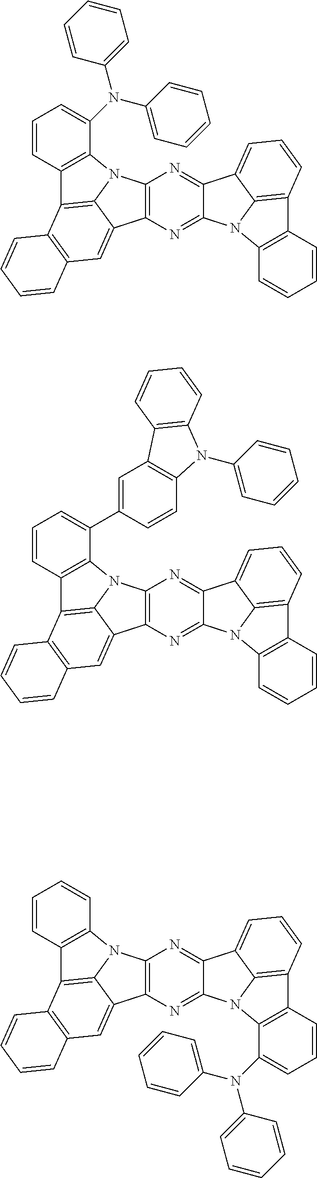 [SMILES: c1ccc(N(c2ccccc2)c2cccc3c4c5ccccc5cc5c6nc7c(nc6n(c23)c54)c2cccc3c4ccccc4n7c32)cc1 ; c1ccc(-n2c3ccccc3c3cc(-c4cccc5c6c7ccccc7cc7c8nc9c(nc8n(c45)c76)c4cccc5c6ccccc6n9c54)ccc32)cc1 ; c1ccc(N(c2ccccc2)c2cccc3c4cccc5c6nc7c(nc6n(c23)c45)c2cc3ccccc3c3c4ccccc4n7c23)cc1]

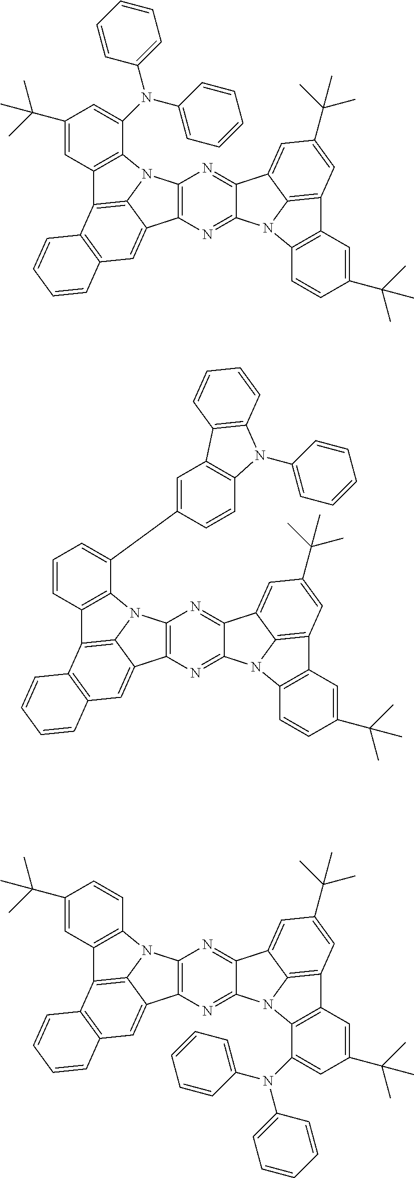

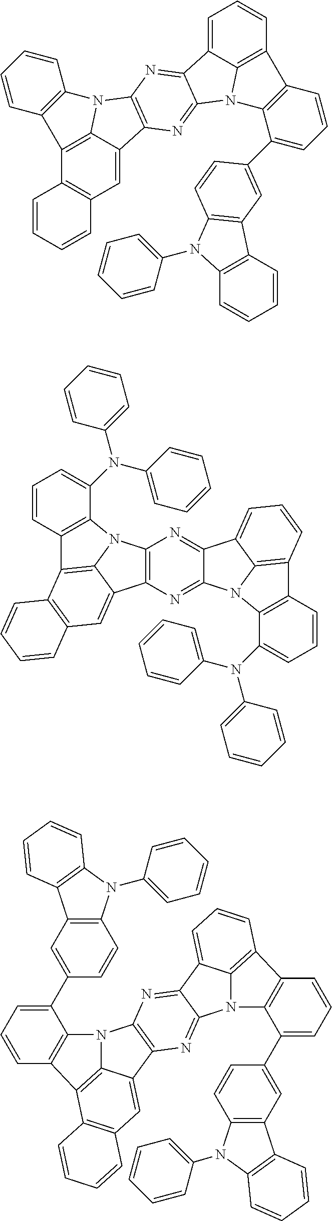 [SMILES: c1ccc(-n2c3ccccc3c3cc(-c4cccc5c6cccc7c8nc9c(nc8n(c45)c67)c4cc5ccccc5c5c6ccccc6n9c45)ccc32)cc1 ; c1ccc(N(c2ccccc2)c2cccc3c4cccc5c6nc7c(nc6n(c23)c45)c2cc3ccccc3c3c4cccc(N(c5ccccc5)c5ccccc5)c4n7c23)cc1 ; c1ccc(-n2c3ccccc3c3cc(-c4cccc5c6cccc7c8nc9c(nc8n(c45)c67)c4cc5ccccc5c5c6cccc(-c7ccc8c(c7)c7ccccc7n8-c7ccccc7)c6n9c45)ccc32)cc1]

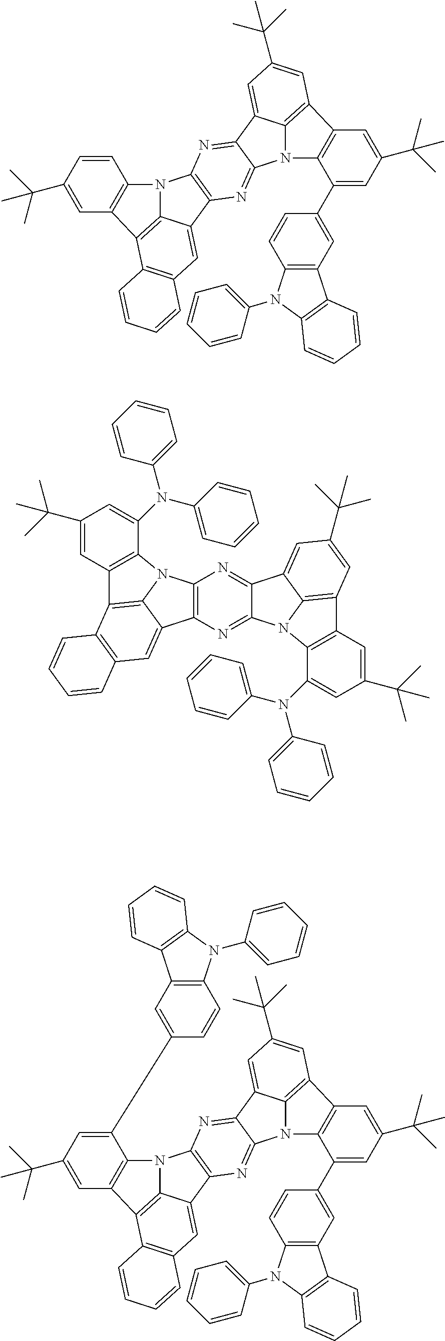

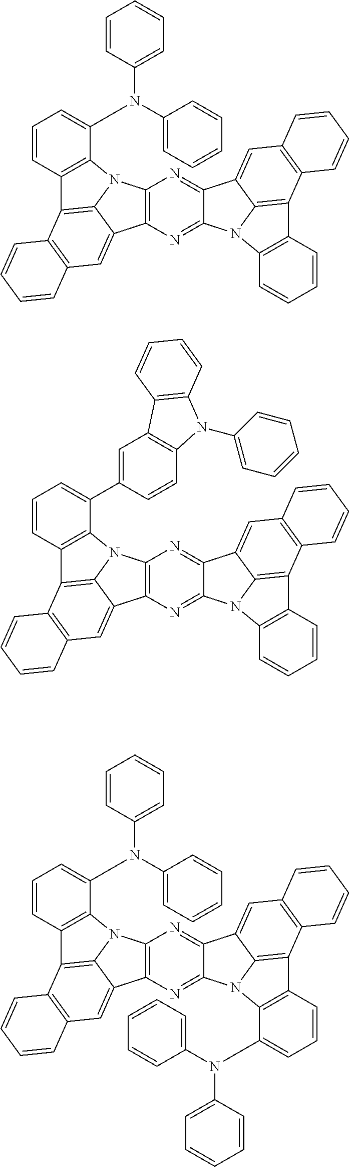 [SMILES: c1ccc(N(c2ccccc2)c2cccc3c4c5ccccc5cc5c6nc7c(nc6n(c23)c54)c2cc3ccccc3c3c4ccccc4n7c23)cc1 ; c1ccc(-n2c3ccccc3c3cc(-c4cccc5c6c7ccccc7cc7c8nc9c(nc8n(c45)c76)c4cc5ccccc5c5c6ccccc6n9c45)ccc32)cc1 ; c1ccc(N(c2ccccc2)c2cccc3c4c5ccccc5cc5c6nc7c(nc6n(c23)c54)c2cc3ccccc3c3c4cccc(N(c5ccccc5)c5ccccc5)c4n7c23)cc1]

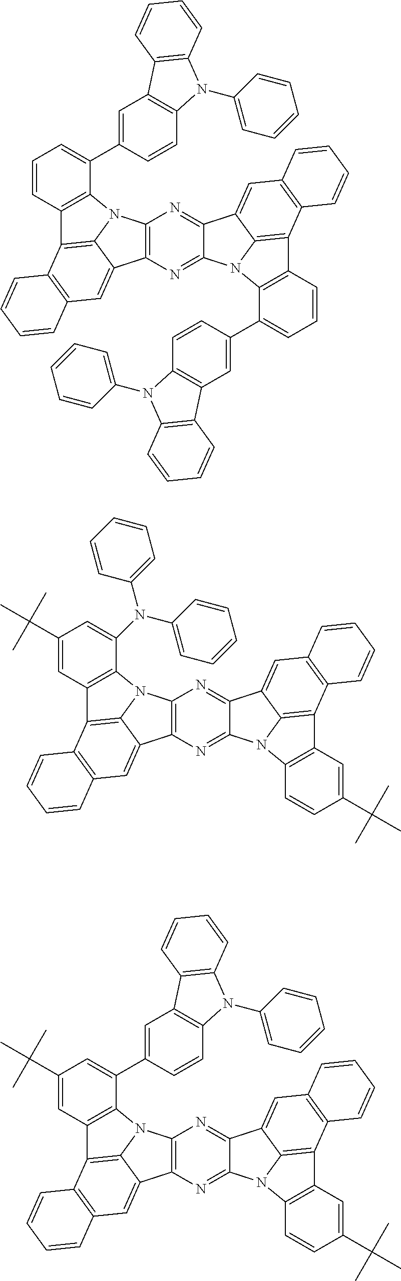

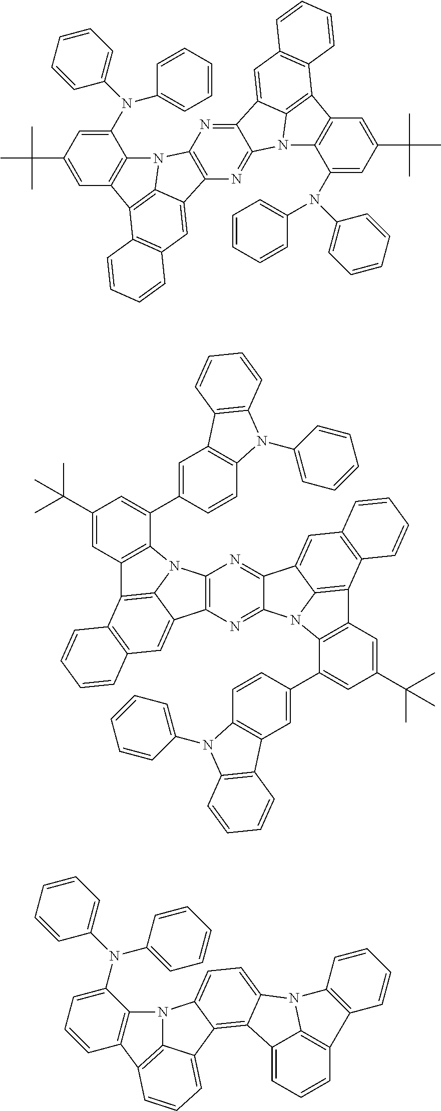

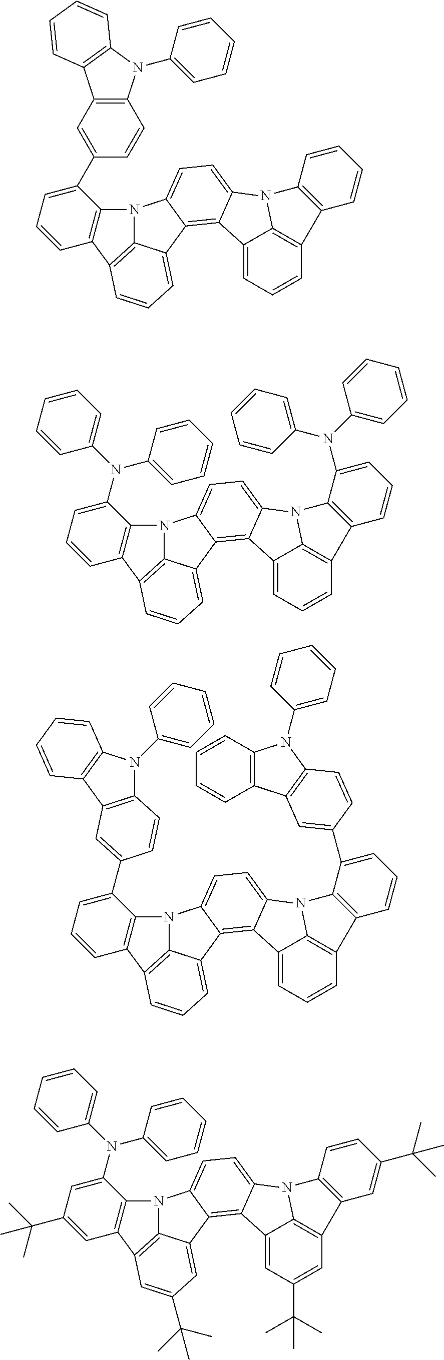

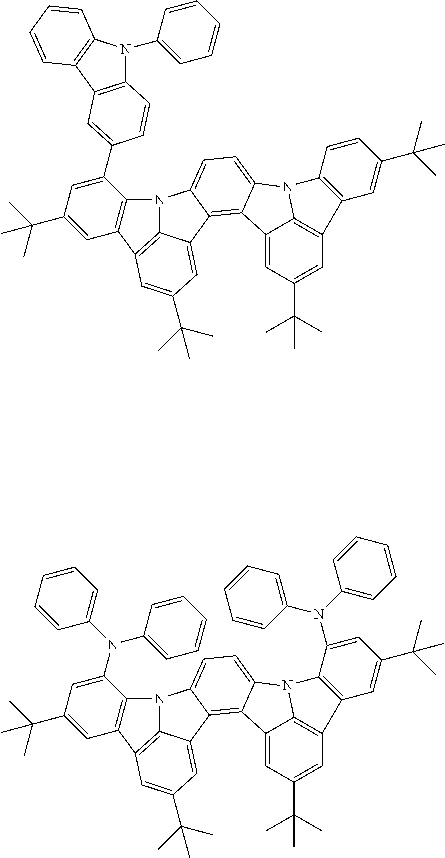

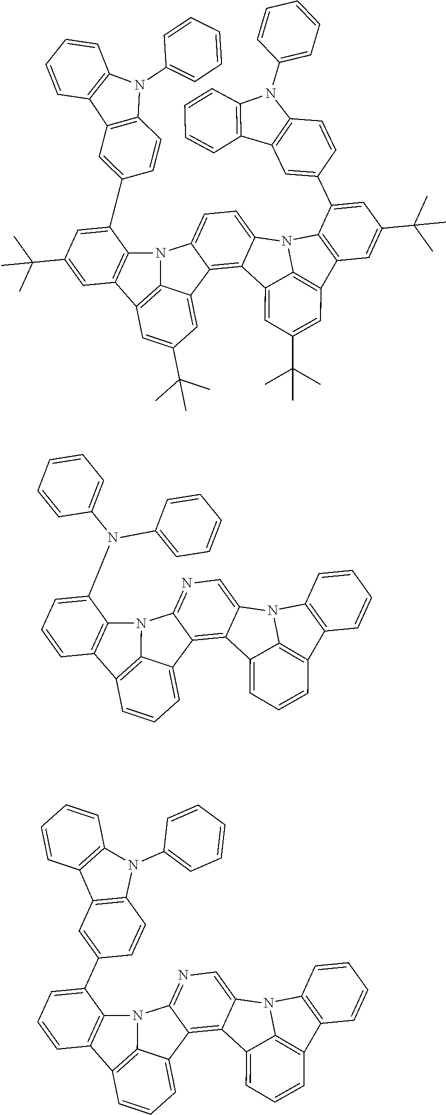 [SMILES: CC(C)(C)c1cc(-c2ccc3c(c2)c2ccccc2n3-c2ccccc2)c2c(c1)c1cc(C(C)(C)C)cc3c4c5c6cc(C(C)(C)C)cc7c8cc(C(C)(C)C)cc(-c9ccc%10c(c9)c9ccccc9n%10-c9ccccc9)c8n(c5ccc4n2c13)c76 ; c1ccc(N(c2ccccc2)c2cccc3c4cccc5c6c7c8cccc9c%10ccccc%10n(c7cnc6n(c23)c45)c98)cc1 ; c1ccc(-n2c3ccccc3c3cc(-c4cccc5c6cccc7c8c9c%10cccc%11c%12ccccc%12n(c9cnc8n(c45)c67)c%11%10)ccc32)cc1]

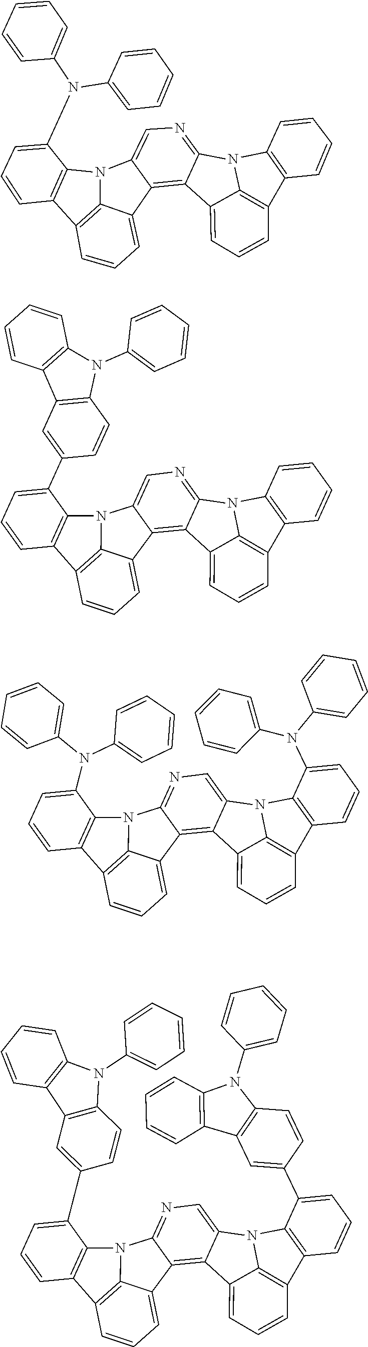 [SMILES: c1ccc(N(c2ccccc2)c2cccc3c4cccc5c6c7c8cccc9c%10ccccc%10n(c7ncc6n(c23)c45)c98)cc1 ; c1ccc(-n2c3ccccc3c3cc(-c4cccc5c6cccc7c8c9c%10cccc%11c%12ccccc%12n(c9ncc8n(c45)c67)c%11%10)ccc32)cc1 ; c1ccc(N(c2ccccc2)c2cccc3c4cccc5c6c7c8cccc9c%10cccc(N(c%11ccccc%11)c%11ccccc%11)c%10n(c7ncc6n(c23)c45)c98)cc1 ; c1ccc(-n2c3ccccc3c3cc(-c4cccc5c6cccc7c8c9c%10cccc%11c%12cccc(-c%13ccc%14c(c%13)c%13ccccc%13n%14-c%13ccccc%13)c%12n(c9ncc8n(c45)c67)c%11%10)ccc32)cc1]

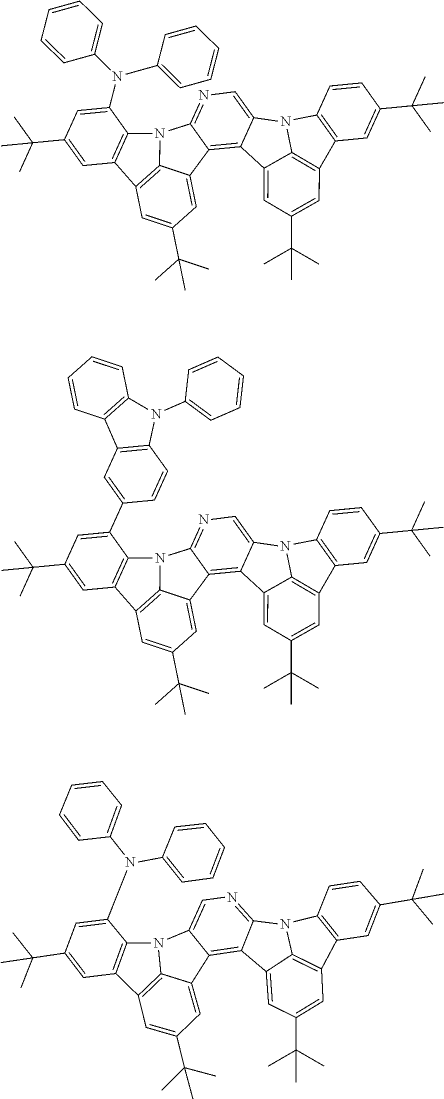

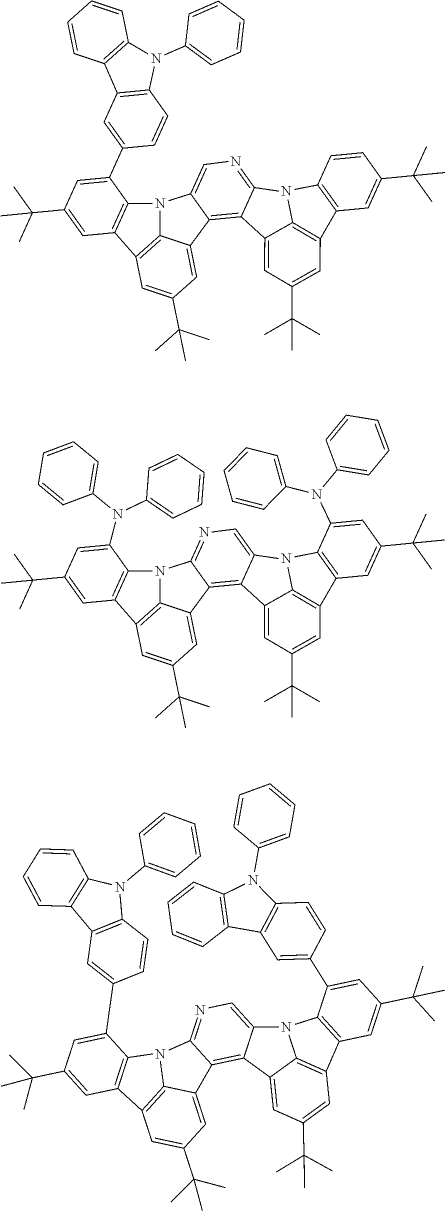

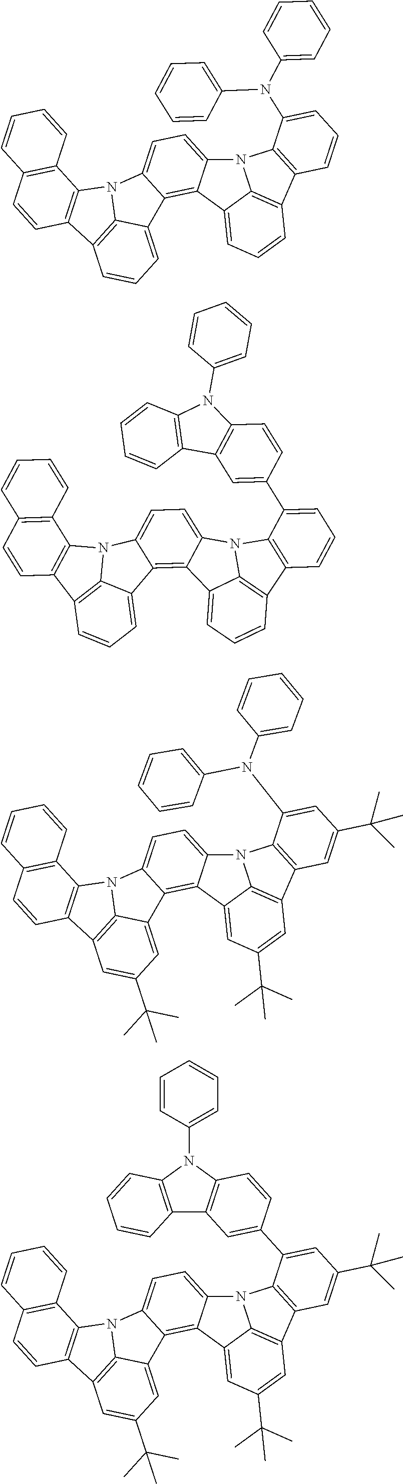

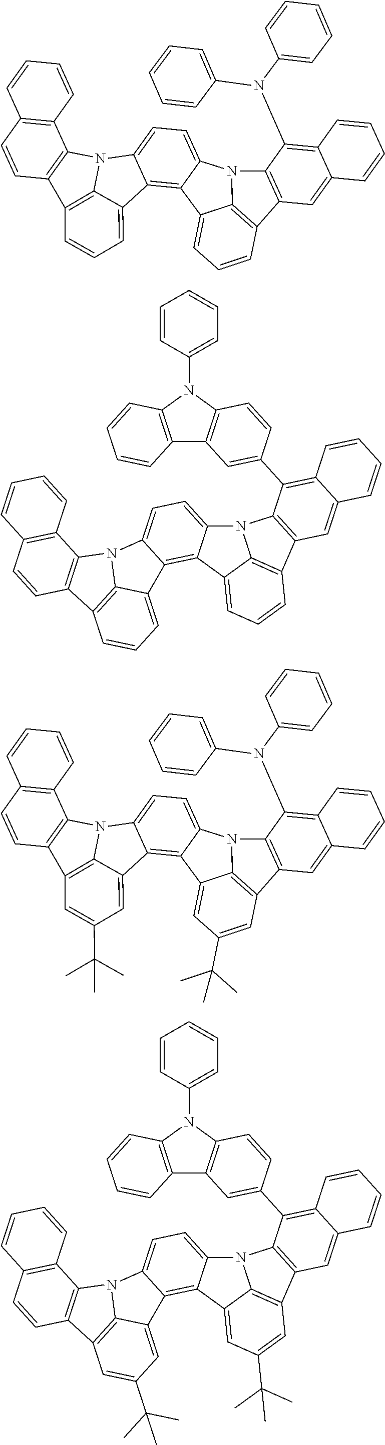

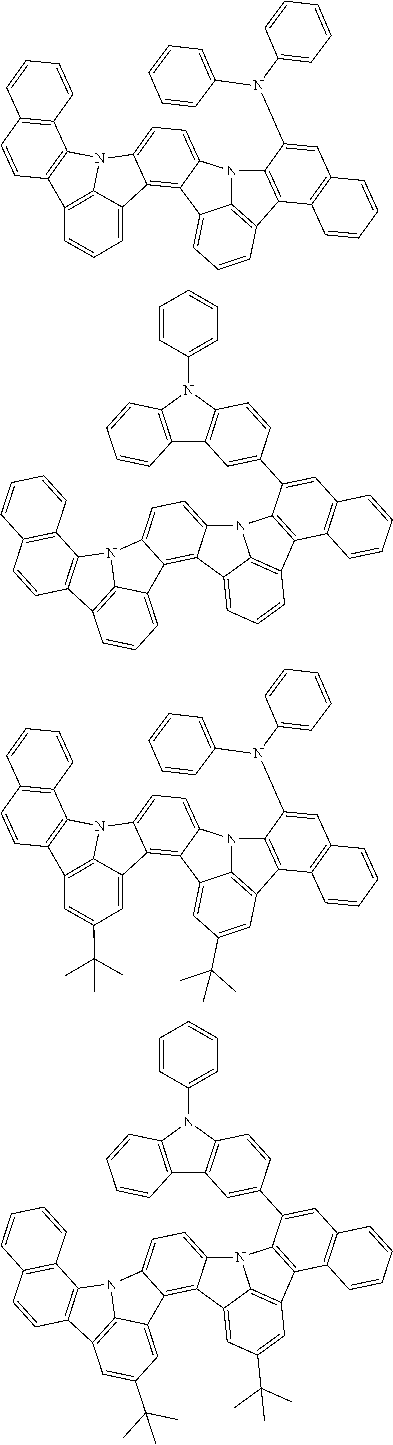

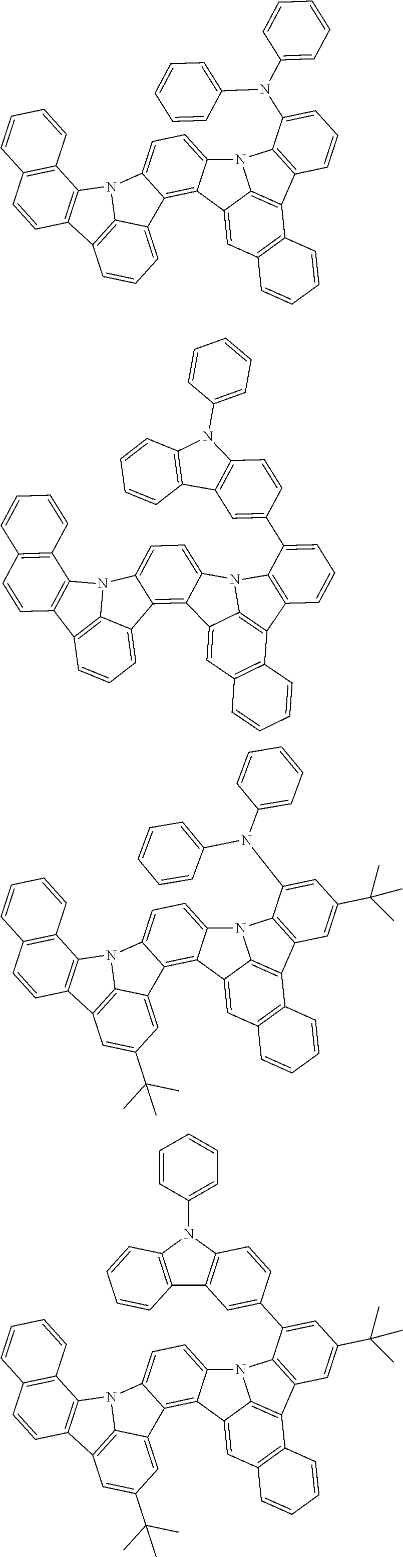

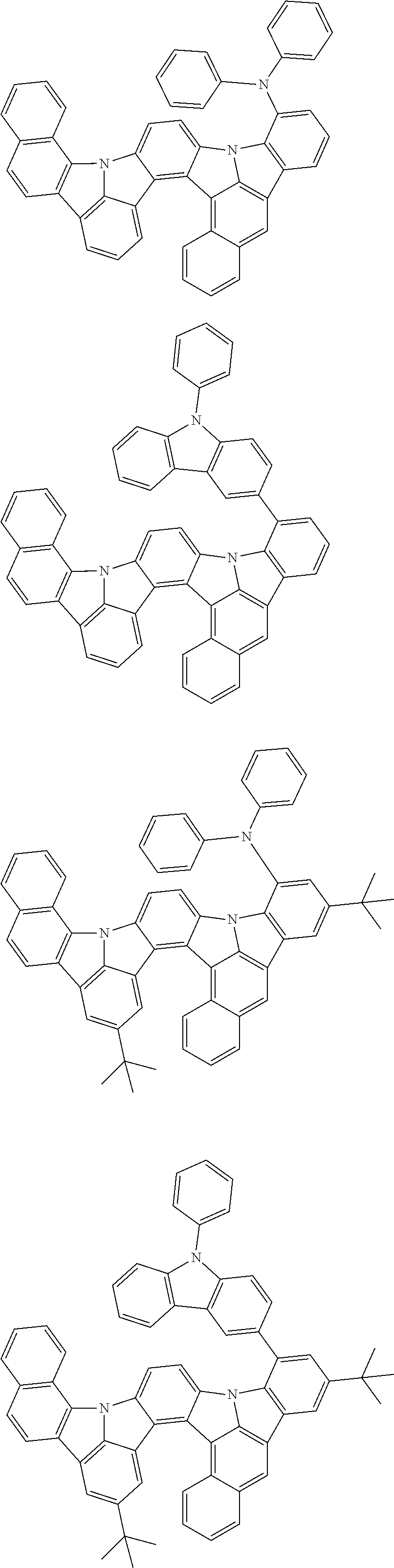

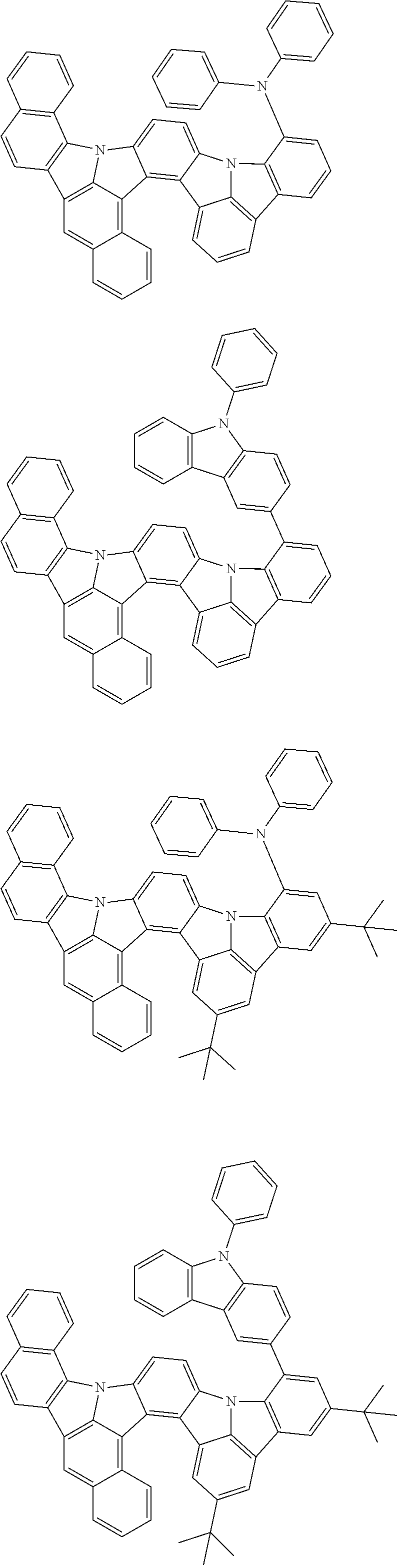 [SMILES: c1ccc(N(c2ccccc2)c2cccc3c4cccc5c6c7c8c9ccccc9cc9c%10ccc%11ccccc%11c%10n(c7ccc6n(c23)c45)c98)cc1 ; c1ccc(-n2c3ccccc3c3cc(-c4cccc5c6cccc7c8c9c%10c%11ccccc%11cc%11c%12ccc%13ccccc%13c%12n(c9ccc8n(c45)c67)c%11%10)ccc32)cc1 ; CC(C)(C)c1cc(N(c2ccccc2)c2ccccc2)c2c(c1)c1cc(C(C)(C)C)cc3c4c5c6c7ccccc7cc7c8ccc9ccccc9c8n(c5ccc4n2c13)c76 ; CC(C)(C)c1cc(-c2ccc3c(c2)c2ccccc2n3-c2ccccc2)c2c(c1)c1cc(C(C)(C)C)cc3c4c5c6c7ccccc7cc7c8ccc9ccccc9c8n(c5ccc4n2c13)c76]

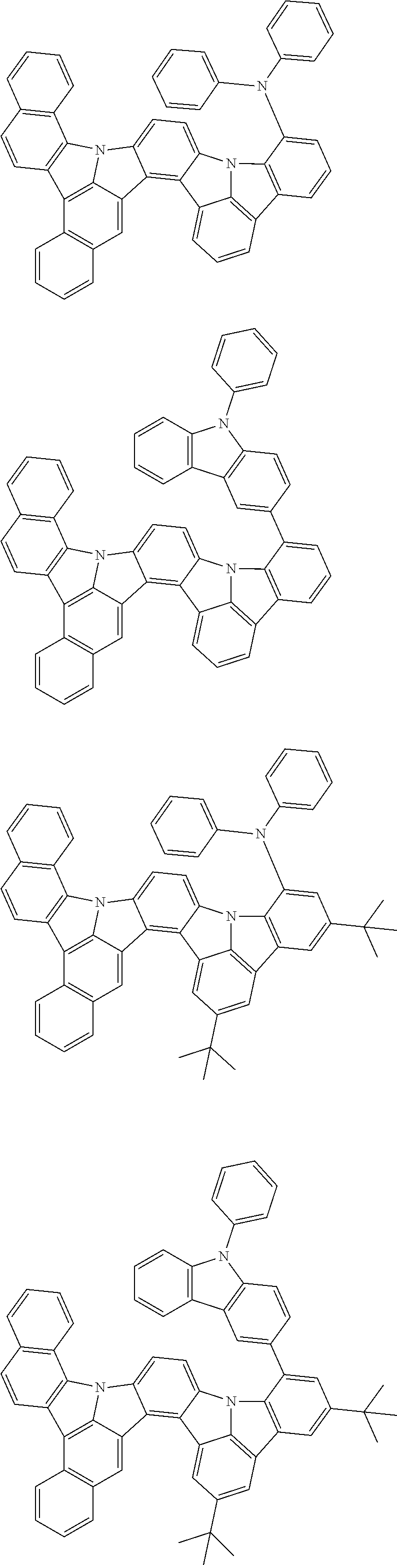 [SMILES: c1ccc(N(c2ccccc2)c2cccc3c4cccc5c6c7c8cc9ccccc9c9c%10ccc%11ccccc%11c%10n(c7ccc6n(c23)c45)c89)cc1 ; c1ccc(-n2c3ccccc3c3cc(-c4cccc5c6cccc7c8c9c%10cc%11ccccc%11c%11c%12ccc%13ccccc%13c%12n(c9ccc8n(c45)c67)c%10%11)ccc32)cc1 ; CC(C)(C)c1cc(N(c2ccccc2)c2ccccc2)c2c(c1)c1cc(C(C)(C)C)cc3c4c5c6cc7ccccc7c7c8ccc9ccccc9c8n(c5ccc4n2c13)c67 ; CC(C)(C)c1cc(-c2ccc3c(c2)c2ccccc2n3-c2ccccc2)c2c(c1)c1cc(C(C)(C)C)cc3c4c5c6cc7ccccc7c7c8ccc9ccccc9c8n(c5ccc4n2c13)c67]

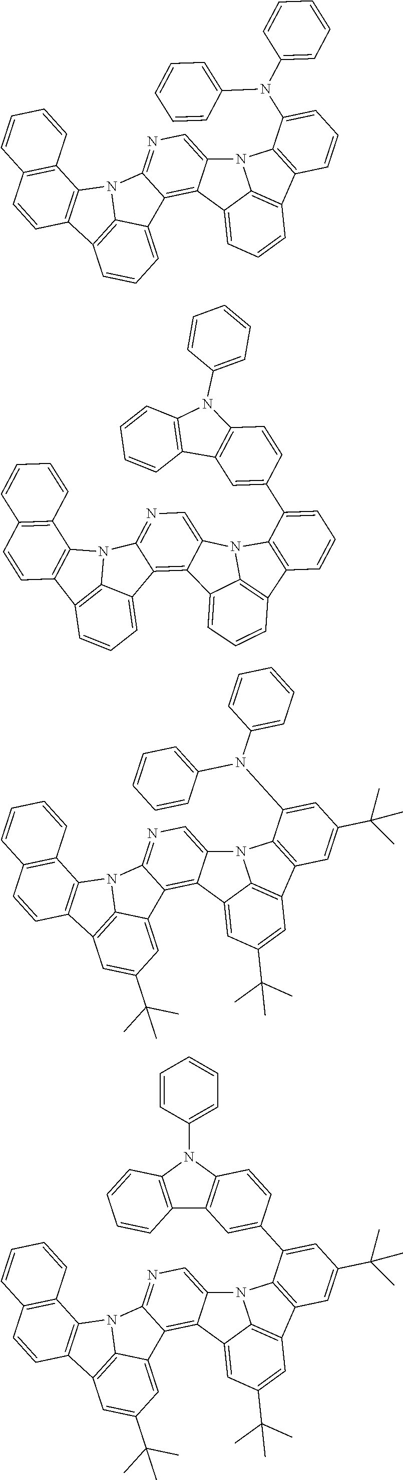

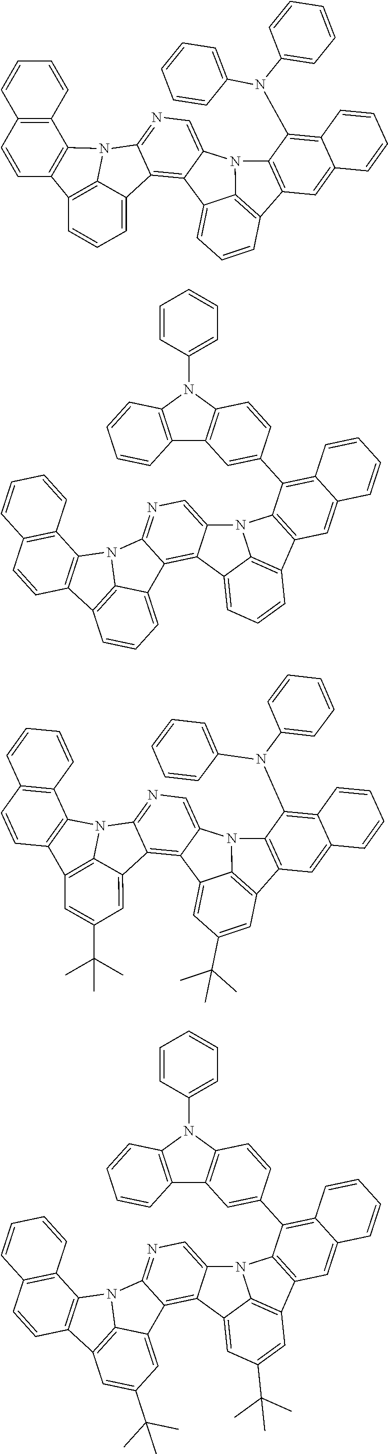

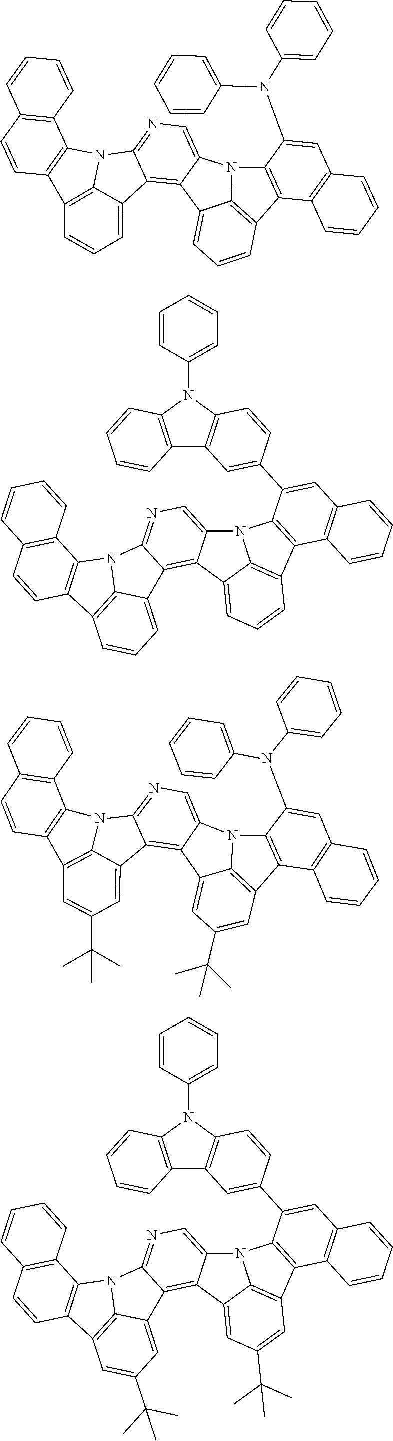

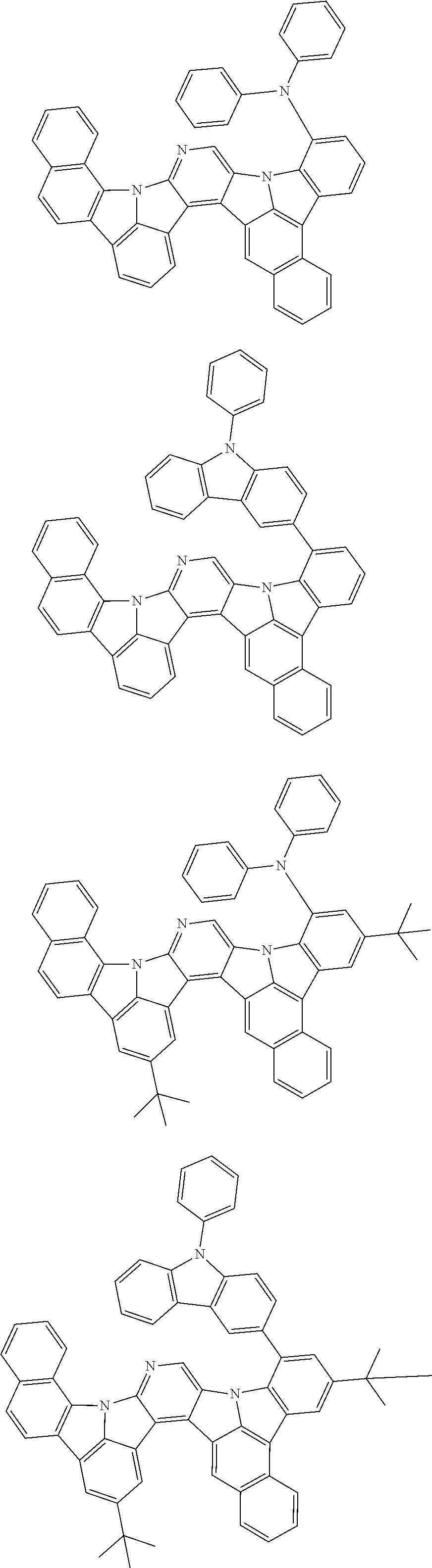

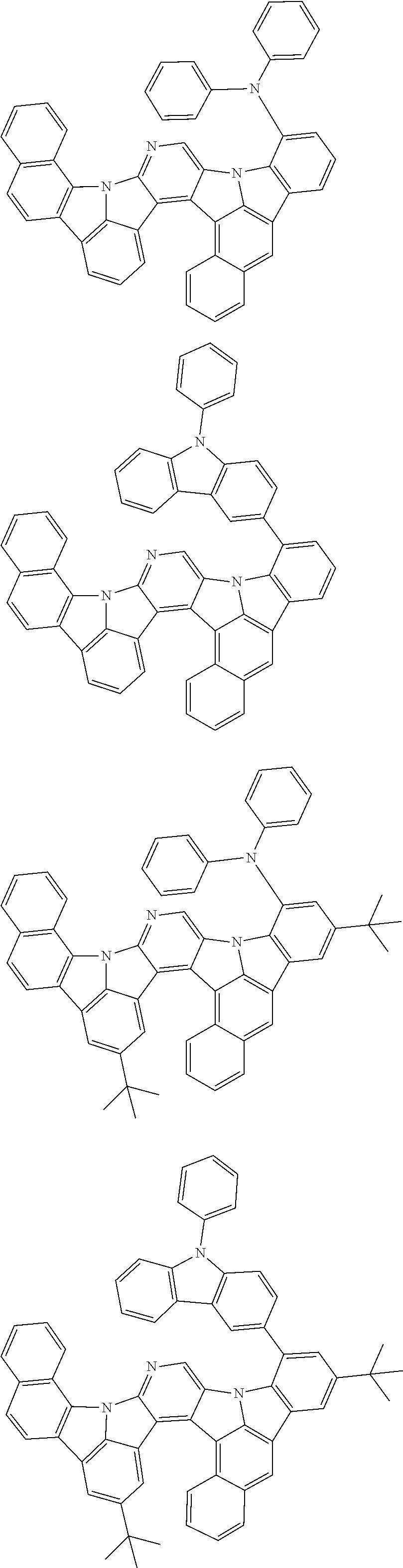

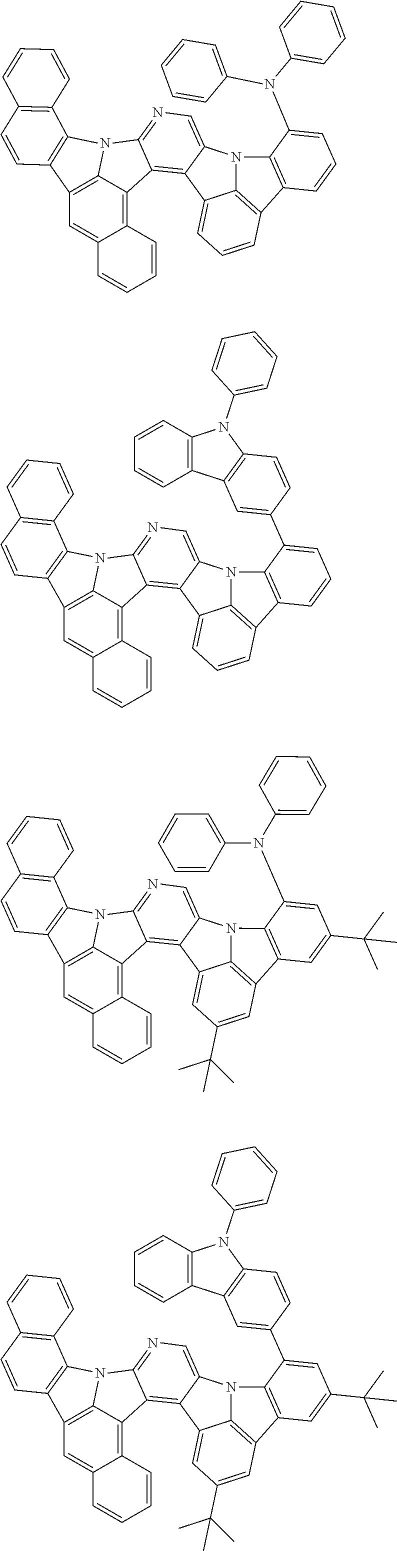 [SMILES: c1ccc(N(c2ccccc2)c2cccc3c4cccc5c6c7c8c9ccccc9cc9c%10ccc%11ccccc%11c%10n(c7ncc6n(c23)c45)c98)cc1 ; c1ccc(-n2c3ccccc3c3cc(-c4cccc5c6cccc7c8c9c%10c%11ccccc%11cc%11c%12ccc%13ccccc%13c%12n(c9ncc8n(c45)c67)c%11%10)ccc32)cc1 ; CC(C)(C)c1cc(N(c2ccccc2)c2ccccc2)c2c(c1)c1cc(C(C)(C)C)cc3c4c5c6c7ccccc7cc7c8ccc9ccccc9c8n(c5ncc4n2c13)c76 ; CC(C)(C)c1cc(-c2ccc3c(c2)c2ccccc2n3-c2ccccc2)c2c(c1)c1cc(C(C)(C)C)cc3c4c5c6c7ccccc7cc7c8ccc9ccccc9c8n(c5ncc4n2c13)c76]

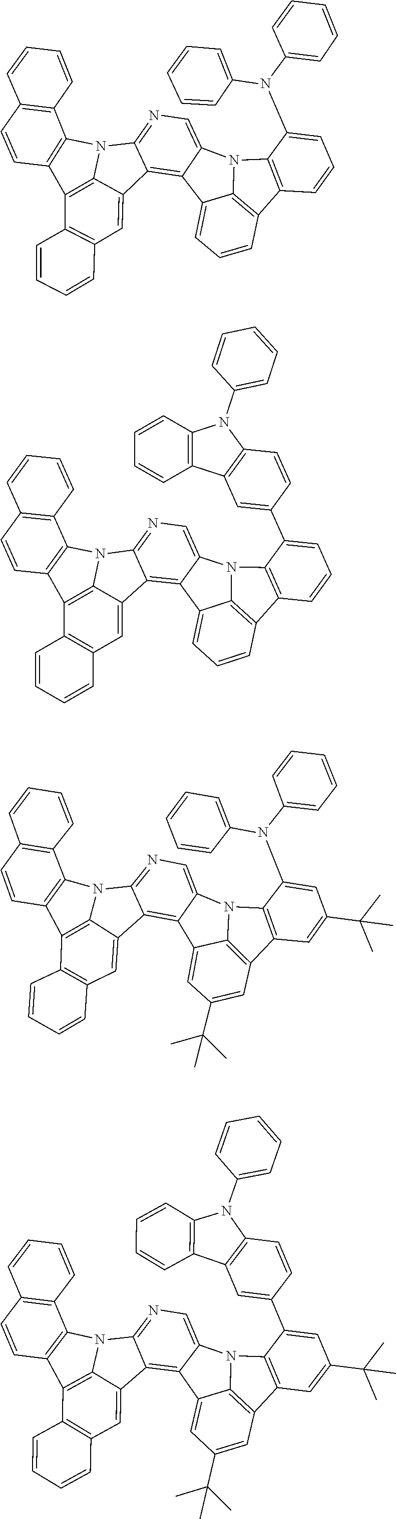 [SMILES: c1ccc(N(c2ccccc2)c2cccc3c4cccc5c6c7c8cc9ccccc9c9c%10ccc%11ccccc%11c%10n(c7ncc6n(c23)c45)c89)cc1 ; c1ccc(-n2c3ccccc3c3cc(-c4cccc5c6cccc7c8c9c%10cc%11ccccc%11c%11c%12ccc%13ccccc%13c%12n(c9ncc8n(c45)c67)c%10%11)ccc32)cc1 ; CC(C)(C)c1cc(N(c2ccccc2)c2ccccc2)c2c(c1)c1cc(C(C)(C)C)cc3c4c5c6cc7ccccc7c7c8ccc9ccccc9c8n(c5ncc4n2c13)c67 ; CC(C)(C)c1cc(-c2ccc3c(c2)c2ccccc2n3-c2ccccc2)c2c(c1)c1cc(C(C)(C)C)cc3c4c5c6cc7ccccc7c7c8ccc9ccccc9c8n(c5ncc4n2c13)c67]

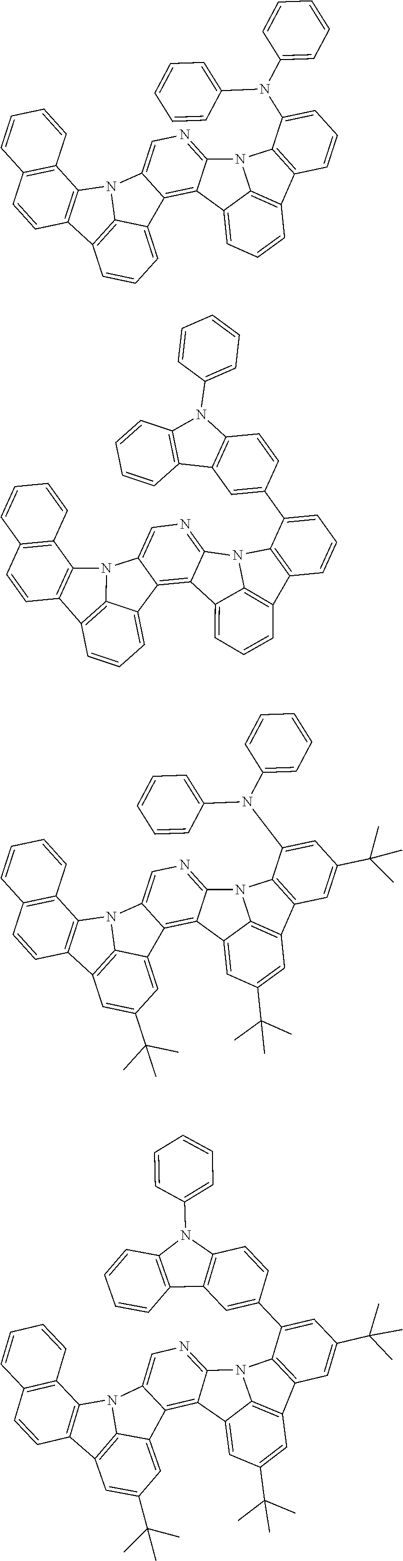 [SMILES: c1ccc(N(c2ccccc2)c2cccc3c4cccc5c6c7c8cccc9c%10ccc%11ccccc%11c%10n(c7cnc6n(c23)c45)c98)cc1 ; c1ccc(-n2c3ccccc3c3cc(-c4cccc5c6cccc7c8c9c%10cccc%11c%12ccc%13ccccc%13c%12n(c9cnc8n(c45)c67)c%11%10)ccc32)cc1 ; CC(C)(C)c1cc(N(c2ccccc2)c2ccccc2)c2c(c1)c1cc(C(C)(C)C)cc3c4c5c6cc(C(C)(C)C)cc7c8ccc9ccccc9c8n(c5cnc4n2c13)c76 ; CC(C)(C)c1cc(-c2ccc3c(c2)c2ccccc2n3-c2ccccc2)c2c(c1)c1cc(C(C)(C)C)cc3c4c5c6cc(C(C)(C)C)cc7c8ccc9ccccc9c8n(c5cnc4n2c13)c76]

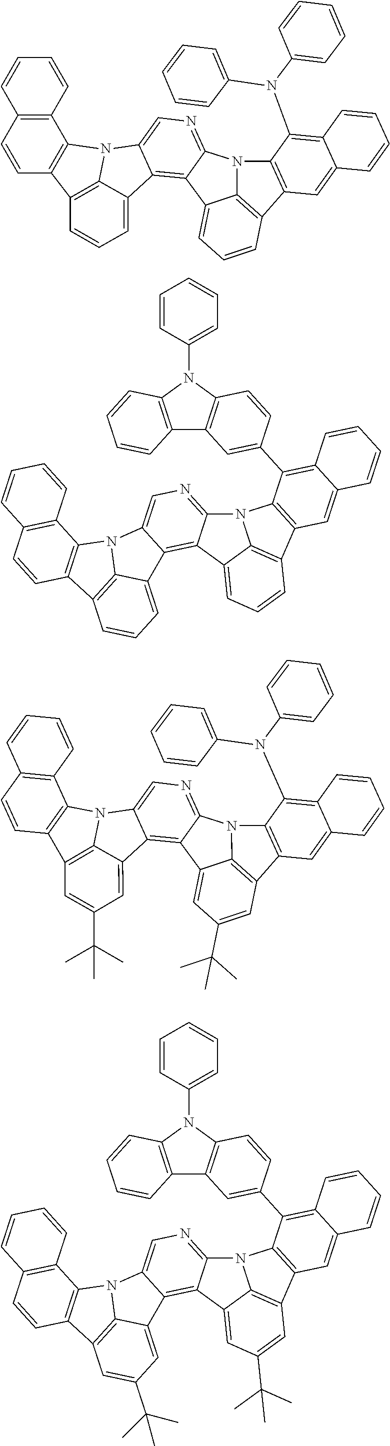

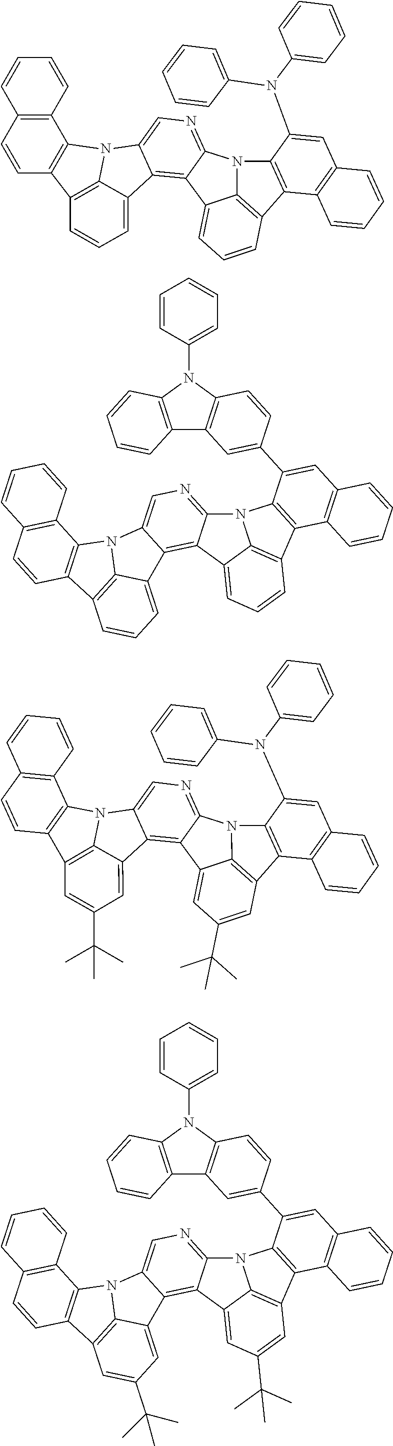

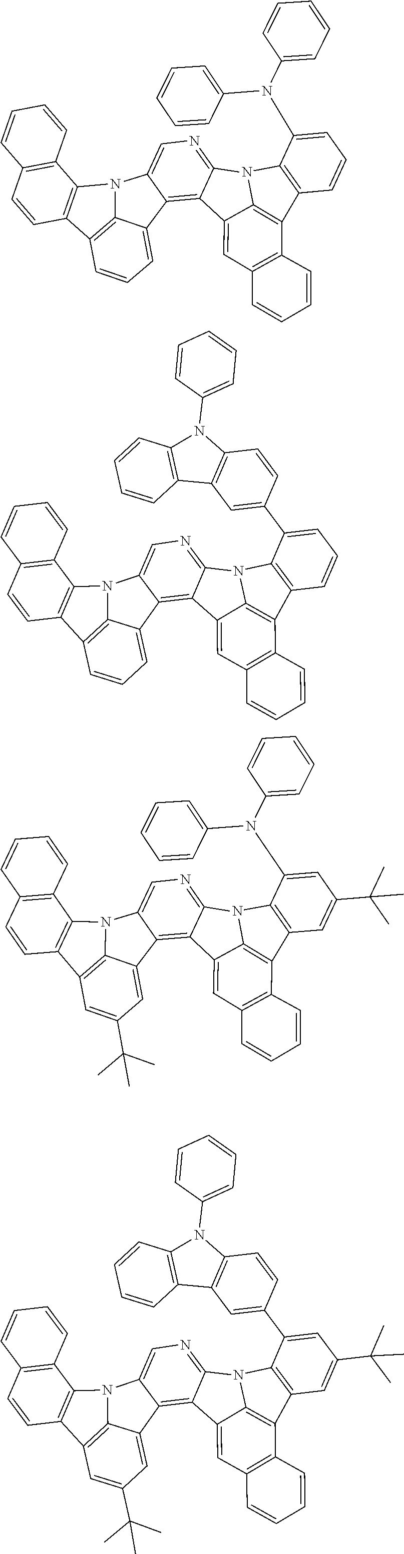

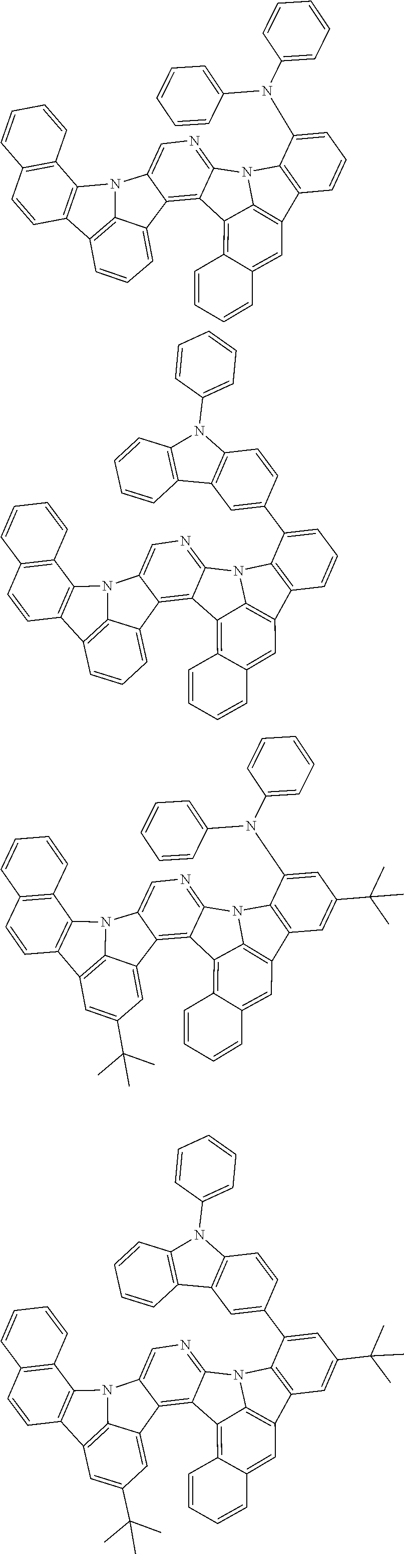

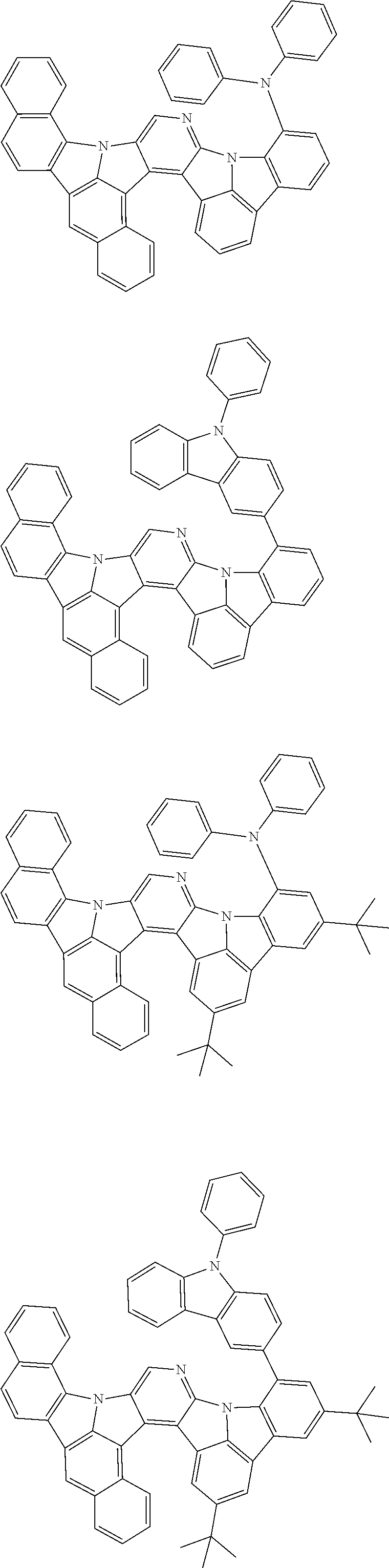 [SMILES: c1ccc(N(c2ccccc2)c2cccc3c4cccc5c6c7c8c9ccccc9cc9c%10ccc%11ccccc%11c%10n(c7cnc6n(c23)c45)c98)cc1 ; c1ccc(-n2c3ccccc3c3cc(-c4cccc5c6cccc7c8c9c%10c%11ccccc%11cc%11c%12ccc%13ccccc%13c%12n(c9cnc8n(c45)c67)c%11%10)ccc32)cc1 ; CC(C)(C)c1cc(N(c2ccccc2)c2ccccc2)c2c(c1)c1cc(C(C)(C)C)cc3c4c5c6c7ccccc7cc7c8ccc9ccccc9c8n(c5cnc4n2c13)c76 ; CC(C)(C)c1cc(-c2ccc3c(c2)c2ccccc2n3-c2ccccc2)c2c(c1)c1cc(C(C)(C)C)cc3c4c5c6c7ccccc7cc7c8ccc9ccccc9c8n(c5cnc4n2c13)c76]

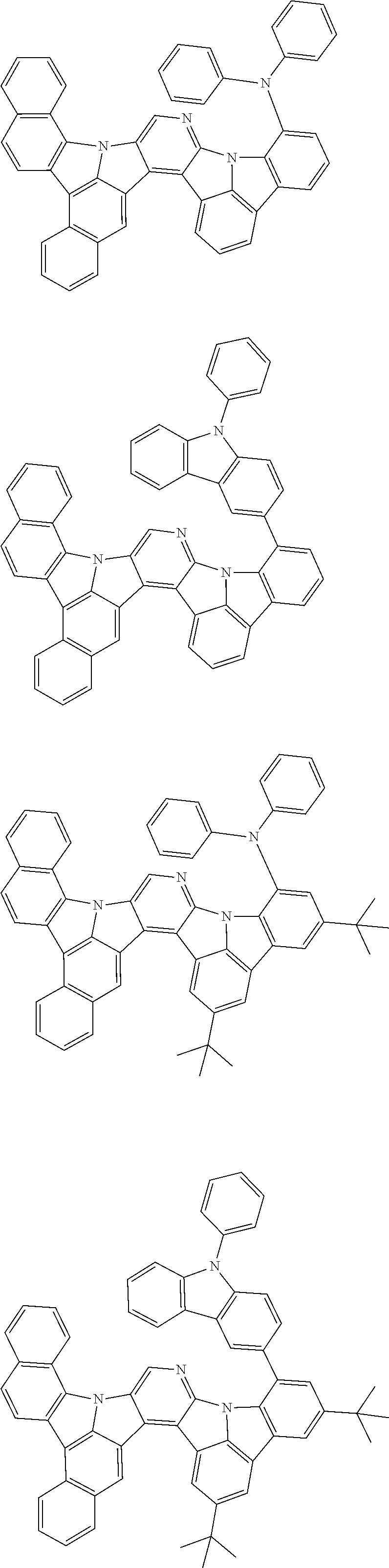 [SMILES: c1ccc(N(c2ccccc2)c2cccc3c4cccc5c6c7c8cc9ccccc9c9c%10ccc%11ccccc%11c%10n(c7cnc6n(c23)c45)c89)cc1 ; c1ccc(-n2c3ccccc3c3cc(-c4cccc5c6cccc7c8c9c%10cc%11ccccc%11c%11c%12ccc%13ccccc%13c%12n(c9cnc8n(c45)c67)c%10%11)ccc32)cc1 ; CC(C)(C)c1cc(N(c2ccccc2)c2ccccc2)c2c(c1)c1cc(C(C)(C)C)cc3c4c5c6cc7ccccc7c7c8ccc9ccccc9c8n(c5cnc4n2c13)c67 ; CC(C)(C)c1cc(-c2ccc3c(c2)c2ccccc2n3-c2ccccc2)c2c(c1)c1cc(C(C)(C)C)cc3c4c5c6cc7ccccc7c7c8ccc9ccccc9c8n(c5cnc4n2c13)c67]

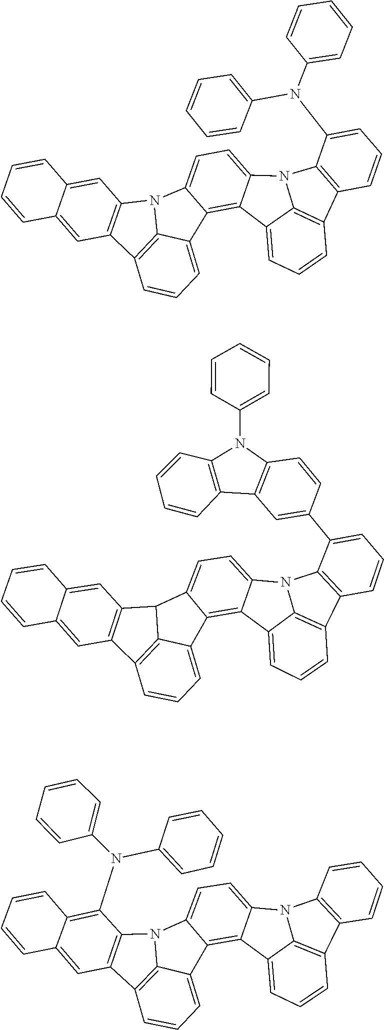 [SMILES: c1ccc(N(c2ccccc2)c2cccc3c4cccc5c6c7c8cccc9c%10cc%11ccccc%11cc%10n(c7ccc6n(c23)c45)c98)cc1 ; c1ccc(-n2c3ccccc3c3cc(-c4cccc5c6cccc7c8c9c(ccc8n(c45)c67)C4c5cc6ccccc6cc5-c5cccc-9c54)ccc32)cc1 ; c1ccc(N(c2ccccc2)c2c3ccccc3cc3c4cccc5c6c7c8cccc9c%10ccccc%10n(c7ccc6n(c23)c45)c98)cc1]

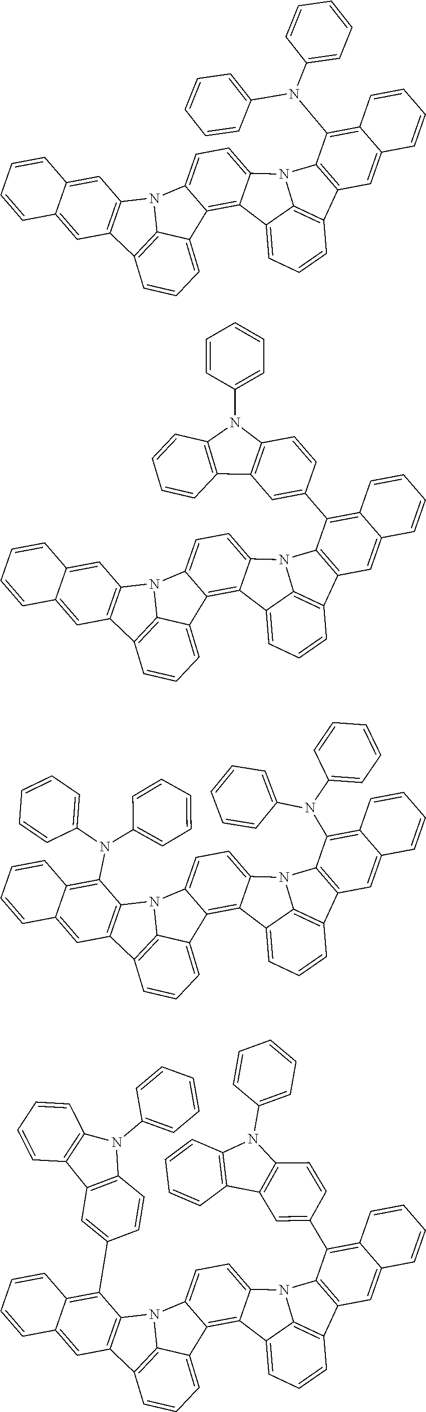

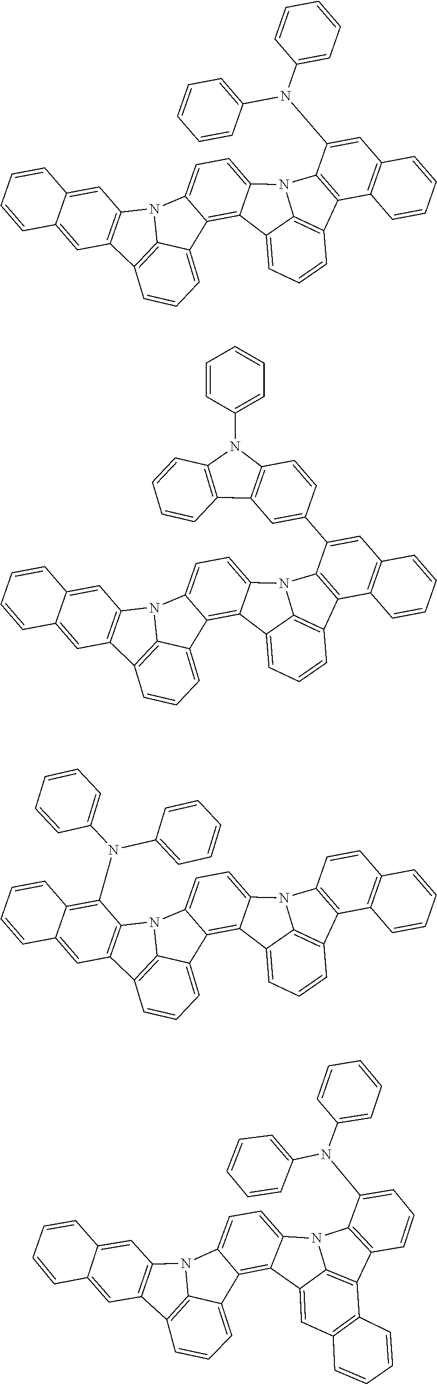 [SMILES: c1ccc(N(c2ccccc2)c2cc3ccccc3c3c4cccc5c6c7c8cccc9c%10cc%11ccccc%11cc%10n(c7ccc6n(c23)c54)c98)cc1 ; c1ccc(-n2c3ccccc3c3cc(-c4cc5ccccc5c5c6cccc7c8c9c%10cccc%11c%12cc%13ccccc%13cc%12n(c9ccc8n(c45)c76)c%11%10)ccc32)cc1 ; c1ccc(N(c2ccccc2)c2c3ccccc3cc3c4cccc5c6c7c8cccc9c%10c%11ccccc%11ccc%10n(c7ccc6n(c23)c45)c98)cc1 ; c1ccc(N(c2ccccc2)c2cccc3c4c5ccccc5cc5c6c7c8cccc9c%10cc%11ccccc%11cc%10n(c7ccc6n(c23)c54)c98)cc1]

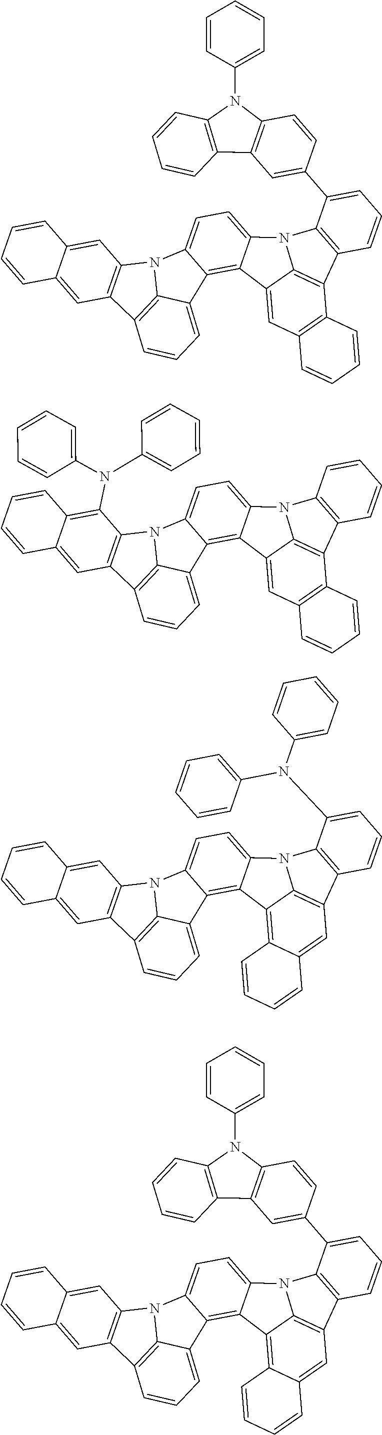

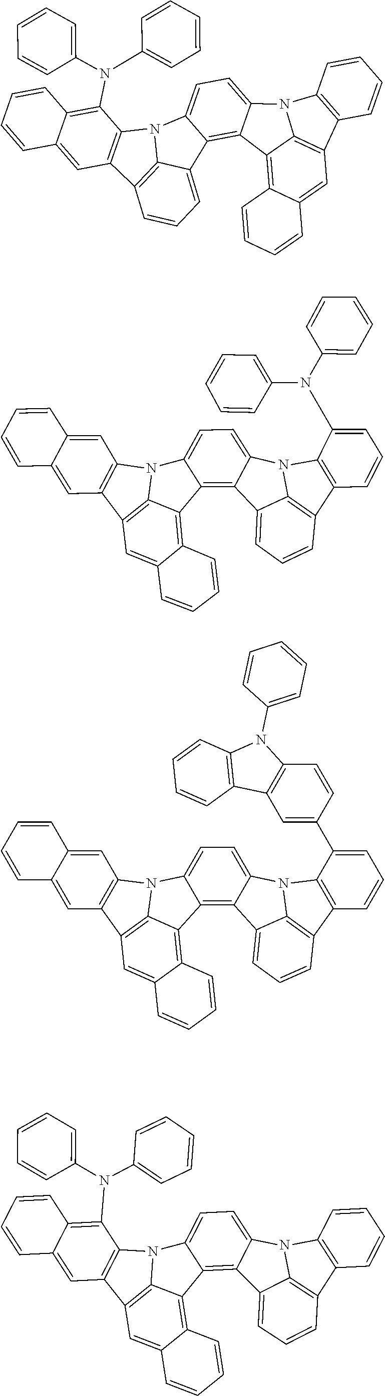 [SMILES: c1ccc(N(c2ccccc2)c2c3ccccc3cc3c4cccc5c6c7c8c9ccccc9cc9c%10ccccc%10n(c7ccc6n(c23)c45)c98)cc1 ; c1ccc(N(c2ccccc2)c2cccc3c4cccc5c6c7c8c9ccccc9cc9c%10cc%11ccccc%11cc%10n(c7ccc6n(c23)c45)c98)cc1 ; c1ccc(-n2c3ccccc3c3cc(-c4cccc5c6cccc7c8c9c%10c%11ccccc%11cc%11c%12cc%13ccccc%13cc%12n(c9ccc8n(c45)c67)c%11%10)ccc32)cc1 ; c1ccc(N(c2ccccc2)c2c3ccccc3cc3c4cc5ccccc5c5c6c7c8cccc9c%10ccccc%10n(c7ccc6n(c23)c45)c98)cc1]

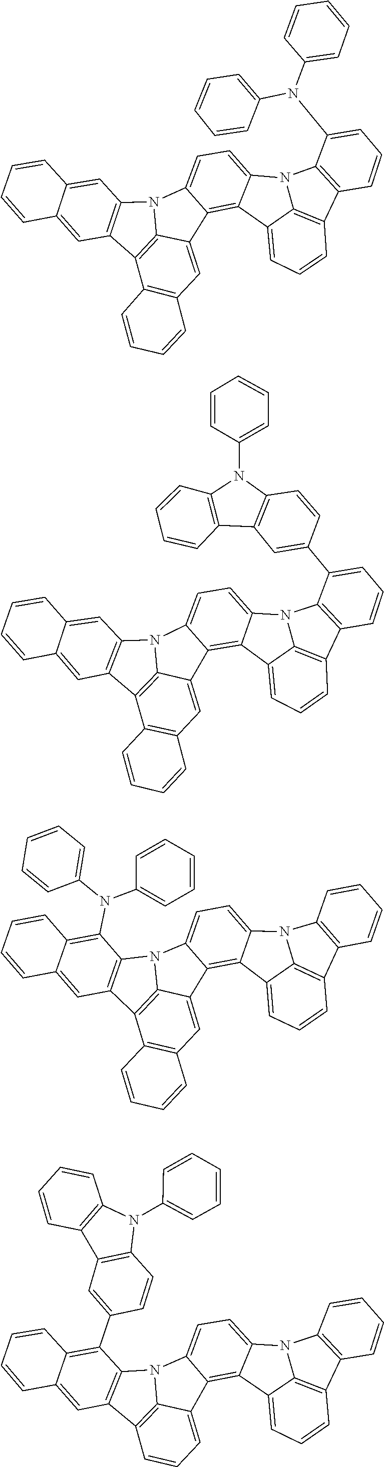

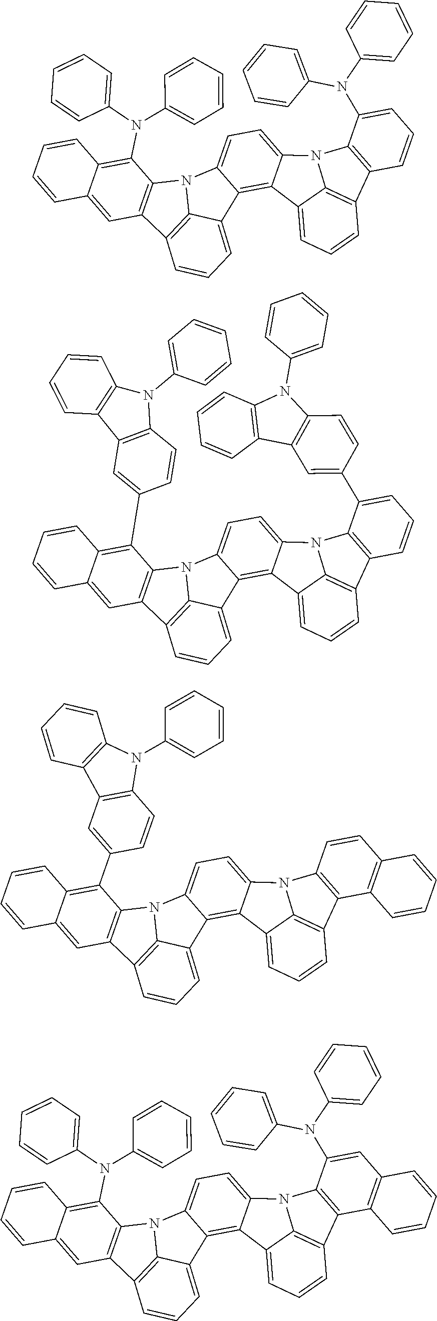

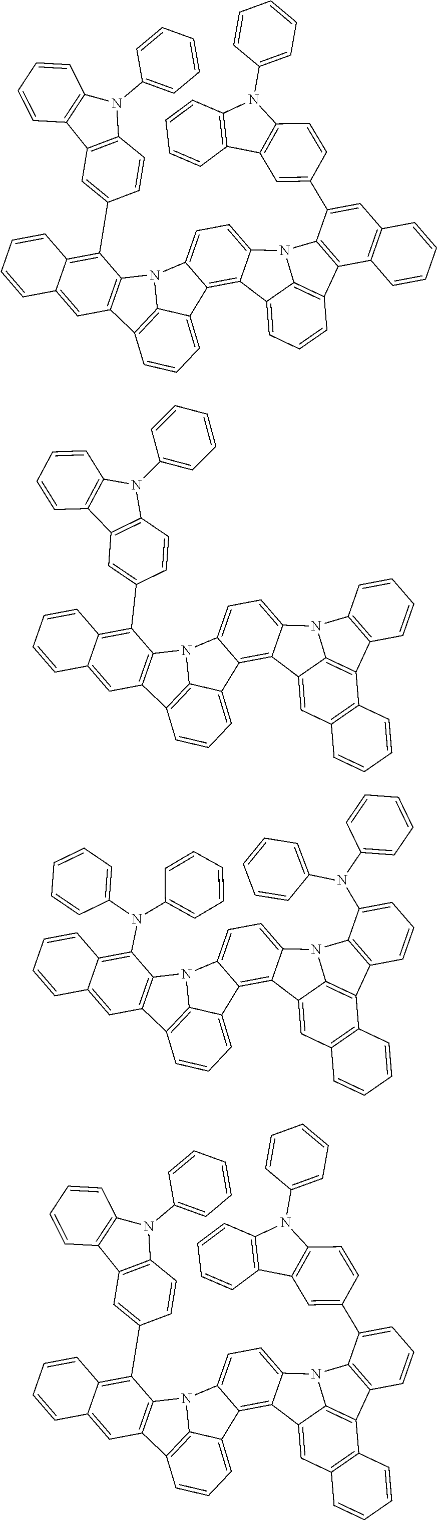

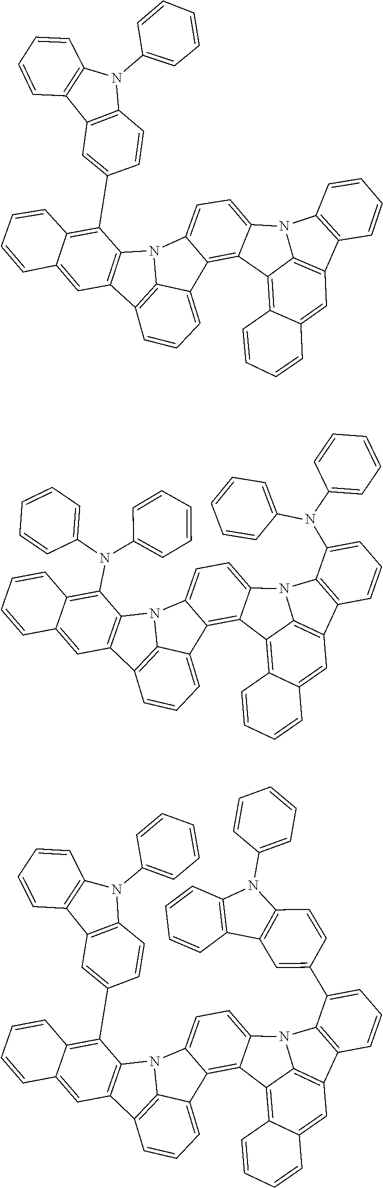 [SMILES: c1ccc(-n2c3ccccc3c3cc(-c4c5ccccc5cc5c6cccc7c8c9c%10c%11ccccc%11cc%11c%12ccccc%12n(c9ccc8n(c45)c67)c%11%10)ccc32)cc1 ; c1ccc(N(c2ccccc2)c2c3ccccc3cc3c4cccc5c6c7c8c9ccccc9cc9c%10cccc(N(c%11ccccc%11)c%11ccccc%11)c%10n(c7ccc6n(c23)c45)c98)cc1 ; c1ccc(-n2c3ccccc3c3cc(-c4c5ccccc5cc5c6cccc7c8c9c%10c%11ccccc%11cc%11c%12cccc(-c%13ccc%14c(c%13)c%13ccccc%13n%14-c%13ccccc%13)c%12n(c9ccc8n(c45)c67)c%11%10)ccc32)cc1]

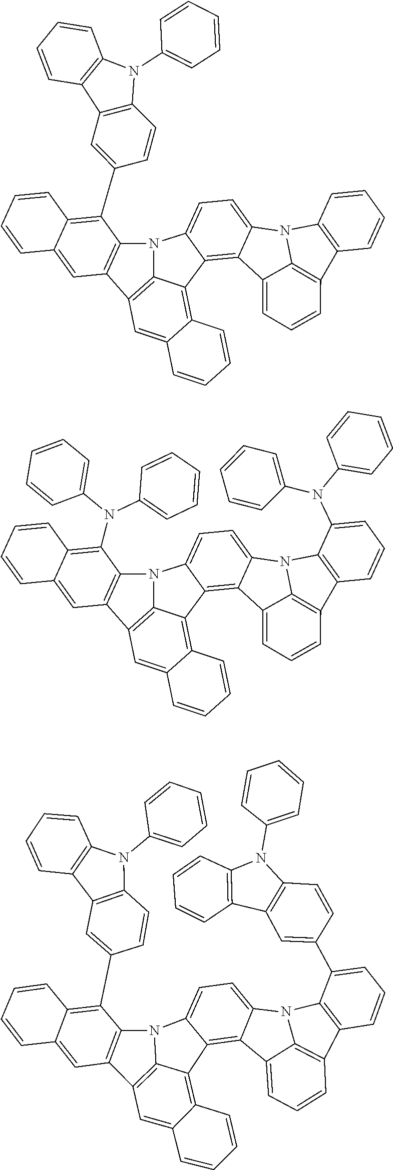 [SMILES: c1ccc(-n2c3ccccc3c3cc(-c4c5ccccc5cc5c6cc7ccccc7c7c8c9c%10cccc%11c%12ccccc%12n(c9ccc8n(c45)c67)c%11%10)ccc32)cc1 ; c1ccc(N(c2ccccc2)c2cccc3c4cccc5c6c7c8c9ccccc9cc9c%10cc%11ccccc%11c(N(c%11ccccc%11)c%11ccccc%11)c%10n(c7ccc6n(c23)c45)c98)cc1 ; c1ccc(-n2c3ccccc3c3cc(-c4cccc5c6cccc7c8c9c%10c%11ccccc%11cc%11c%12cc%13ccccc%13c(-c%13ccc%14c(c%13)c%13ccccc%13n%14-c%13ccccc%13)c%12n(c9ccc8n(c45)c67)c%11%10)ccc32)cc1]

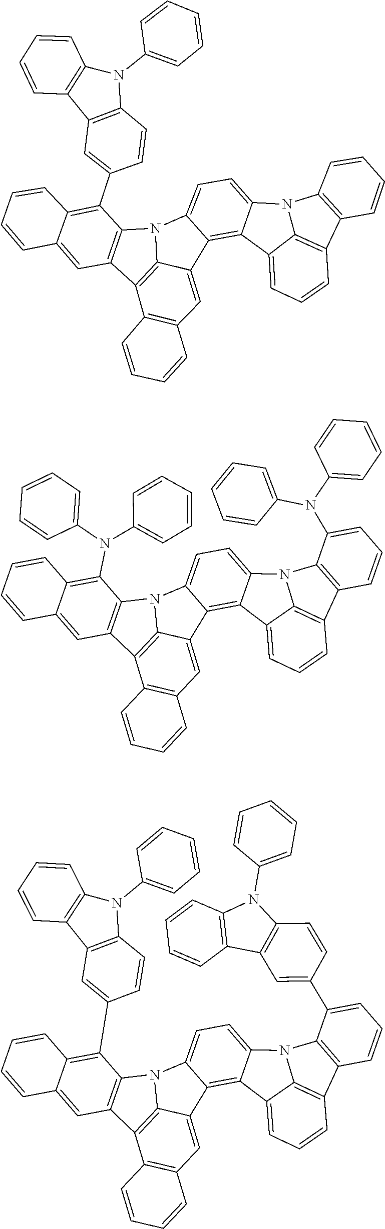 [SMILES: c1ccc(-n2c3ccccc3c3cc(-c4c5ccccc5cc5c6c7ccccc7cc7c8c9c%10cccc%11c%12ccccc%12n(c9ccc8n(c45)c76)c%11%10)ccc32)cc1 ; c1ccc(N(c2ccccc2)c2cccc3c4cccc5c6c7c8cc9ccccc9c9c%10cc%11ccccc%11c(N(c%11ccccc%11)c%11ccccc%11)c%10n(c7ccc6n(c23)c45)c89)cc1 ; c1ccc(-n2c3ccccc3c3cc(-c4cccc5c6cccc7c8c9c%10cc%11ccccc%11c%11c%12cc%13ccccc%13c(-c%13ccc%14c(c%13)c%13ccccc%13n%14-c%13ccccc%13)c%12n(c9ccc8n(c45)c67)c%10%11)ccc32)cc1]

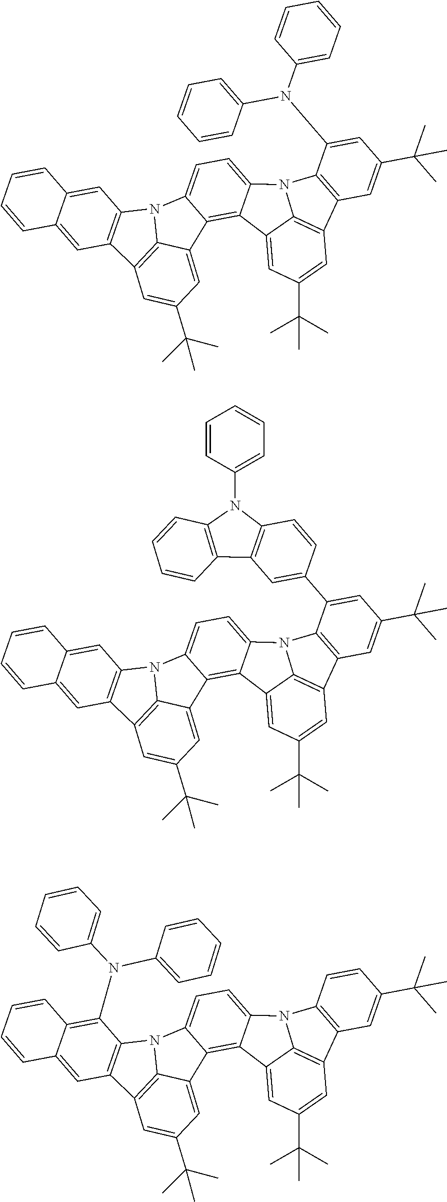 [SMILES: CC(C)(C)c1cc(N(c2ccccc2)c2ccccc2)c2c(c1)c1cc(C(C)(C)C)cc3c4c5c6cc(C(C)(C)C)cc7c8cc9ccccc9cc8n(c5ccc4n2c13)c76 ; CC(C)(C)c1cc(-c2ccc3c(c2)c2ccccc2n3-c2ccccc2)c2c(c1)c1cc(C(C)(C)C)cc3c4c5c6cc(C(C)(C)C)cc7c8cc9ccccc9cc8n(c5ccc4n2c13)c76 ; CC(C)(C)c1ccc2c(c1)c1cc(C(C)(C)C)cc3c4c5c6cc(C(C)(C)C)cc7c8cc9ccccc9c(N(c9ccccc9)c9ccccc9)c8n(c5ccc4n2c13)c76]

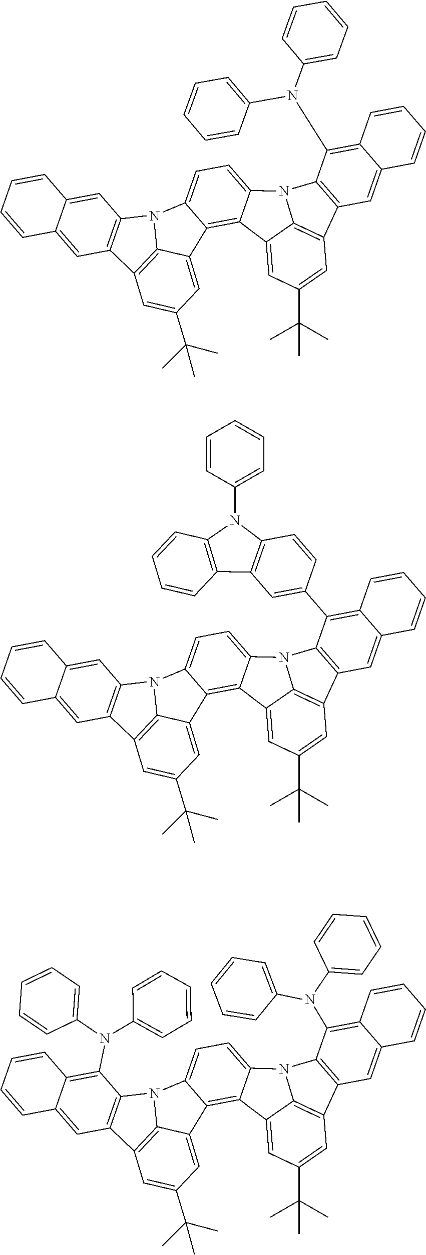 [SMILES: CC(C)(C)c1cc2c3cc4ccccc4cc3n3c4ccc5c(c6cc(C(C)(C)C)cc7c8cc9ccccc9c(N(c9ccccc9)c9ccccc9)c8n5c76)c4c(c1)c23 ; CC(C)(C)c1cc2c3cc4ccccc4cc3n3c4ccc5c(c6cc(C(C)(C)C)cc7c8cc9ccccc9c(-c9ccc%10c(c9)c9ccccc9n%10-c9ccccc9)c8n5c76)c4c(c1)c23 ; CC(C)(C)c1cc2c3cc4ccccc4c(N(c4ccccc4)c4ccccc4)c3n3c4ccc5c(c6cc(C(C)(C)C)cc7c8cc9ccccc9c(N(c9ccccc9)c9ccccc9)c8n5c76)c4c(c1)c23]

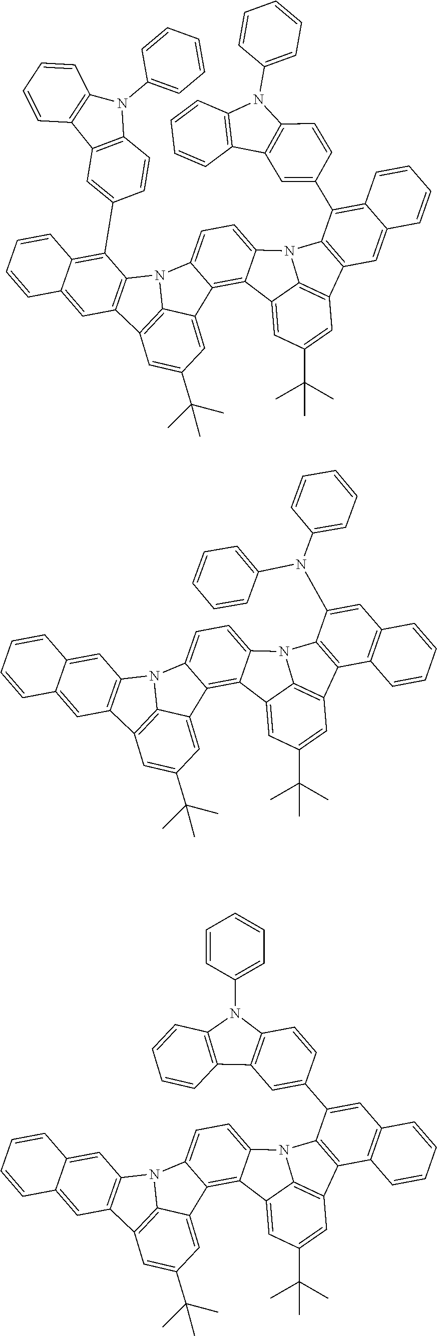 [SMILES: CC(C)(C)c1cc2c3cc4ccccc4c(-c4ccc5c(c4)c4ccccc4n5-c4ccccc4)c3n3c4ccc5c(c6cc(C(C)(C)C)cc7c8cc9ccccc9c(-c9ccc%10c(c9)c9ccccc9n%10-c9ccccc9)c8n5c76)c4c(c1)c23 ; CC(C)(C)c1cc2c3cc4ccccc4cc3n3c4ccc5c(c6cc(C(C)(C)C)cc7c8c9ccccc9cc(N(c9ccccc9)c9ccccc9)c8n5c67)c4c(c1)c23 ; CC(C)(C)c1cc2c3cc4ccccc4cc3n3c4ccc5c(c6cc(C(C)(C)C)cc7c8c9ccccc9cc(-c9ccc%10c(c9)c9ccccc9n%10-c9ccccc9)c8n5c67)c4c(c1)c23]

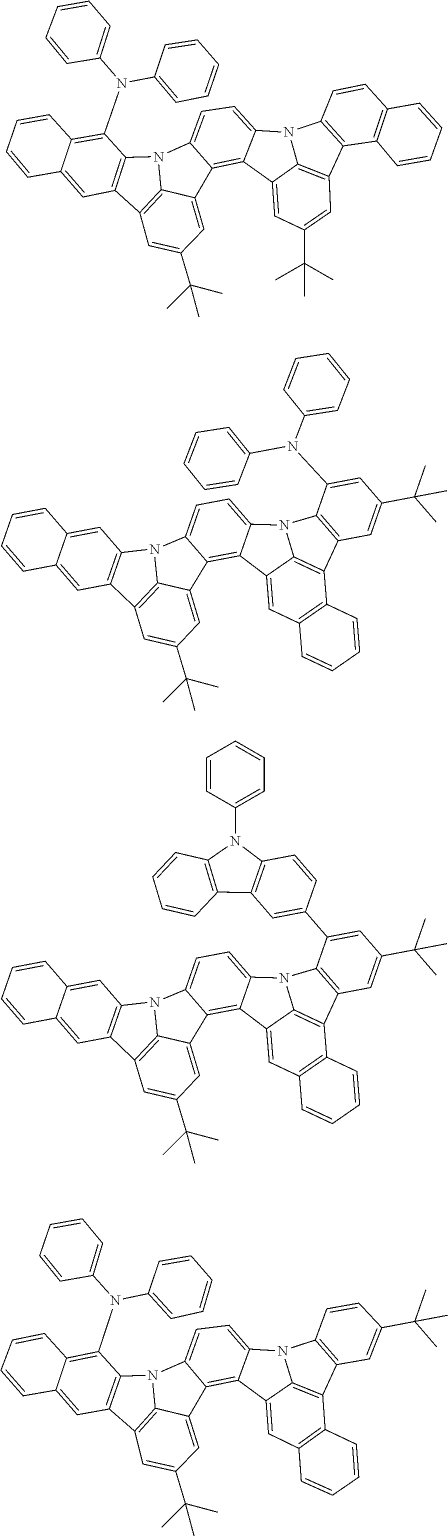 [SMILES: CC(C)(C)c1cc2c3c4ccccc4ccc3n3c4ccc5c(c6cc(C(C)(C)C)cc7c8cc9ccccc9c(N(c9ccccc9)c9ccccc9)c8n5c76)c4c(c1)c23 ; CC(C)(C)c1cc(N(c2ccccc2)c2ccccc2)c2c(c1)c1c3ccccc3cc3c4c5c6cc(C(C)(C)C)cc7c8cc9ccccc9cc8n(c5ccc4n2c31)c76 ; CC(C)(C)c1cc(-c2ccc3c(c2)c2ccccc2n3-c2ccccc2)c2c(c1)c1c3ccccc3cc3c4c5c6cc(C(C)(C)C)cc7c8cc9ccccc9cc8n(c5ccc4n2c31)c76 ; CC(C)(C)c1ccc2c(c1)c1c3ccccc3cc3c4c5c6cc(C(C)(C)C)cc7c8cc9ccccc9c(N(c9ccccc9)c9ccccc9)c8n(c5ccc4n2c31)c76]

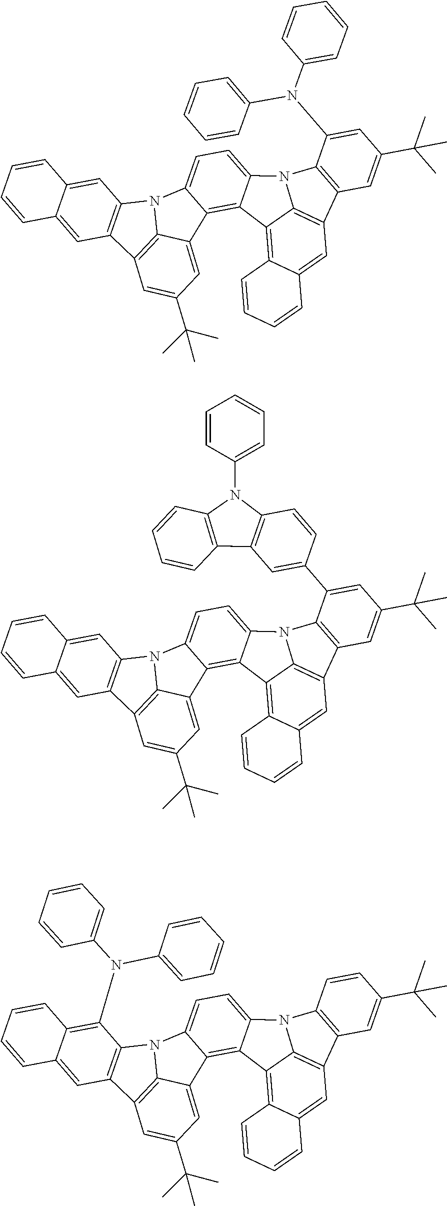 [SMILES: CC(C)(C)c1cc(N(c2ccccc2)c2ccccc2)c2c(c1)c1cc3ccccc3c3c4c5c6cc(C(C)(C)C)cc7c8cc9ccccc9cc8n(c5ccc4n2c13)c76 ; CC(C)(C)c1cc(-c2ccc3c(c2)c2ccccc2n3-c2ccccc2)c2c(c1)c1cc3ccccc3c3c4c5c6cc(C(C)(C)C)cc7c8cc9ccccc9cc8n(c5ccc4n2c13)c76 ; CC(C)(C)c1ccc2c(c1)c1cc3ccccc3c3c4c5c6cc(C(C)(C)C)cc7c8cc9ccccc9c(N(c9ccccc9)c9ccccc9)c8n(c5ccc4n2c13)c76]

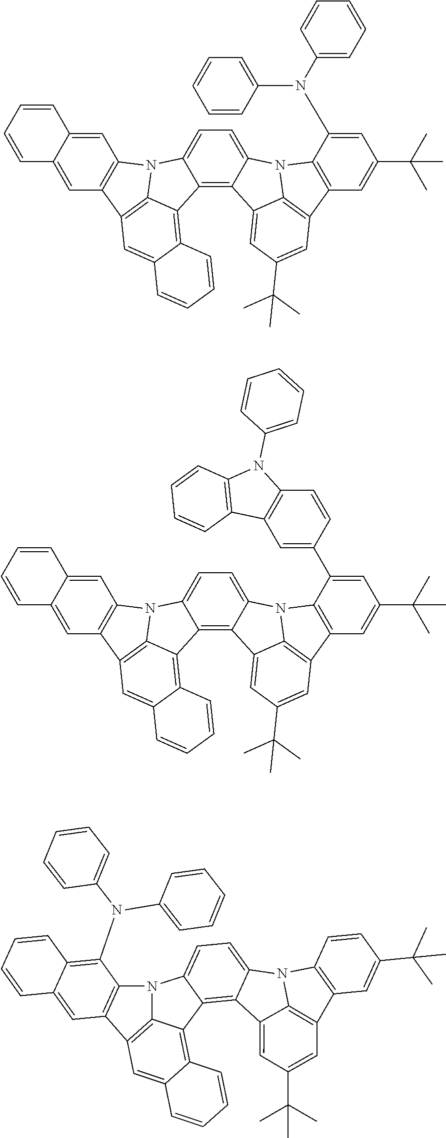

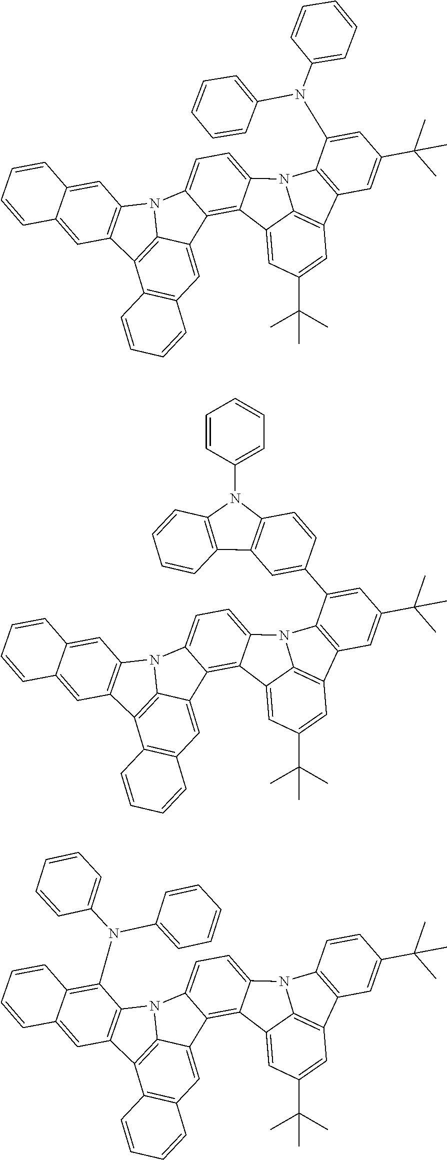 [SMILES: CC(C)(C)c1cc(N(c2ccccc2)c2ccccc2)c2c(c1)c1cc(C(C)(C)C)cc3c4c5c6cc7ccccc7c7c8cc9ccccc9cc8n(c5ccc4n2c13)c67 ; CC(C)(C)c1cc(-c2ccc3c(c2)c2ccccc2n3-c2ccccc2)c2c(c1)c1cc(C(C)(C)C)cc3c4c5c6cc7ccccc7c7c8cc9ccccc9cc8n(c5ccc4n2c13)c67 ; CC(C)(C)c1ccc2c(c1)c1cc(C(C)(C)C)cc3c4c5c6cc7ccccc7c7c8cc9ccccc9c(N(c9ccccc9)c9ccccc9)c8n(c5ccc4n2c13)c67]

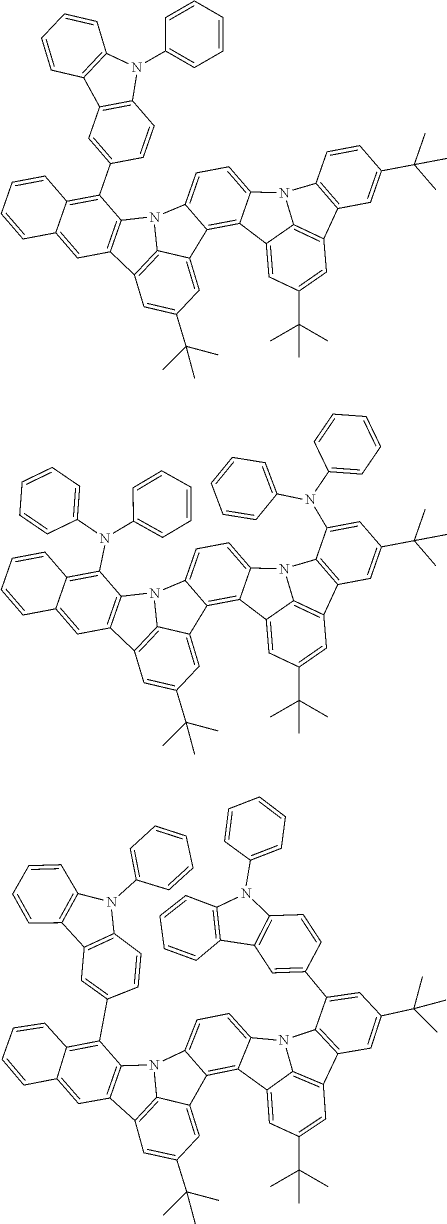 [SMILES: CC(C)(C)c1ccc2c(c1)c1cc(C(C)(C)C)cc3c4c5c6cc(C(C)(C)C)cc7c8cc9ccccc9c(-c9ccc%10c(c9)c9ccccc9n%10-c9ccccc9)c8n(c5ccc4n2c13)c76 ; CC(C)(C)c1cc(N(c2ccccc2)c2ccccc2)c2c(c1)c1cc(C(C)(C)C)cc3c4c5c6cc(C(C)(C)C)cc7c8cc9ccccc9c(N(c9ccccc9)c9ccccc9)c8n(c5ccc4n2c13)c76 ; CC(C)(C)c1cc(-c2ccc3c(c2)c2ccccc2n3-c2ccccc2)c2c(c1)c1cc(C(C)(C)C)cc3c4c5c6cc(C(C)(C)C)cc7c8cc9ccccc9c(-c9ccc%10c(c9)c9ccccc9n%10-c9ccccc9)c8n(c5ccc4n2c13)c76]

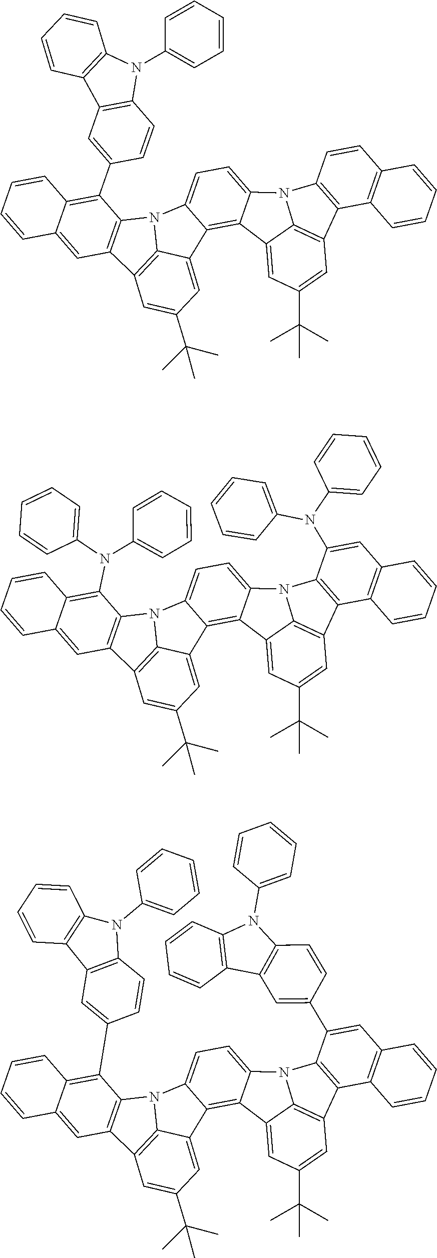 [SMILES: CC(C)(C)c1cc2c3c4ccccc4ccc3n3c4ccc5c(c6cc(C(C)(C)C)cc7c8cc9ccccc9c(-c9ccc%10c(c9)c9ccccc9n%10-c9ccccc9)c8n5c76)c4c(c1)c23 ; CC(C)(C)c1cc2c3cc4ccccc4c(N(c4ccccc4)c4ccccc4)c3n3c4ccc5c(c6cc(C(C)(C)C)cc7c8c9ccccc9cc(N(c9ccccc9)c9ccccc9)c8n5c67)c4c(c1)c23 ; CC(C)(C)c1cc2c3cc4ccccc4c(-c4ccc5c(c4)c4ccccc4n5-c4ccccc4)c3n3c4ccc5c(c6cc(C(C)(C)C)cc7c8c9ccccc9cc(-c9ccc%10c(c9)c9ccccc9n%10-c9ccccc9)c8n5c67)c4c(c1)c23]

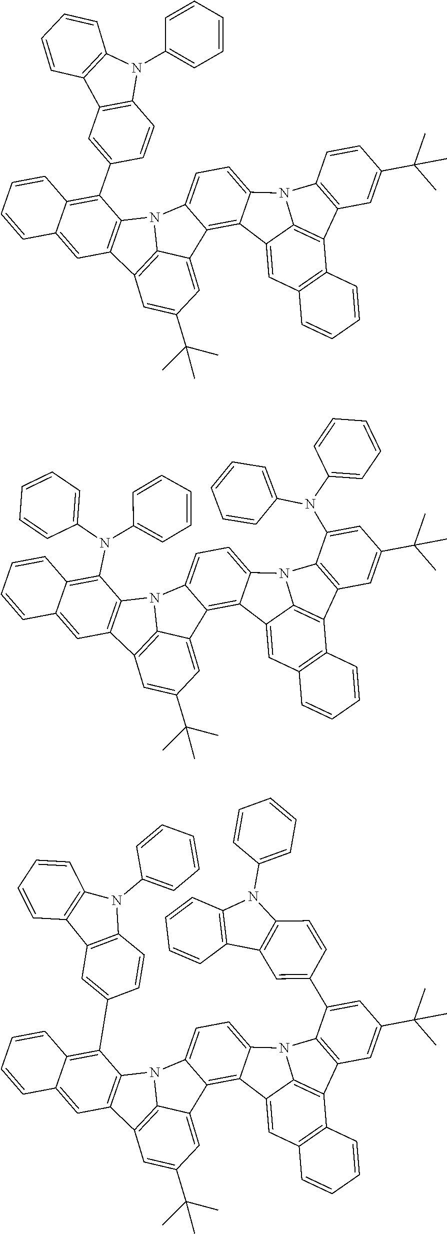 [SMILES: CC(C)(C)c1ccc2c(c1)c1c3ccccc3cc3c4c5c6cc(C(C)(C)C)cc7c8cc9ccccc9c(-c9ccc%10c(c9)c9ccccc9n%10-c9ccccc9)c8n(c5ccc4n2c31)c76 ; CC(C)(C)c1cc(N(c2ccccc2)c2ccccc2)c2c(c1)c1c3ccccc3cc3c4c5c6cc(C(C)(C)C)cc7c8cc9ccccc9c(N(c9ccccc9)c9ccccc9)c8n(c5ccc4n2c31)c76 ; CC(C)(C)c1cc(-c2ccc3c(c2)c2ccccc2n3-c2ccccc2)c2c(c1)c1c3ccccc3cc3c4c5c6cc(C(C)(C)C)cc7c8cc9ccccc9c(-c9ccc%10c(c9)c9ccccc9n%10-c9ccccc9)c8n(c5ccc4n2c31)c76]

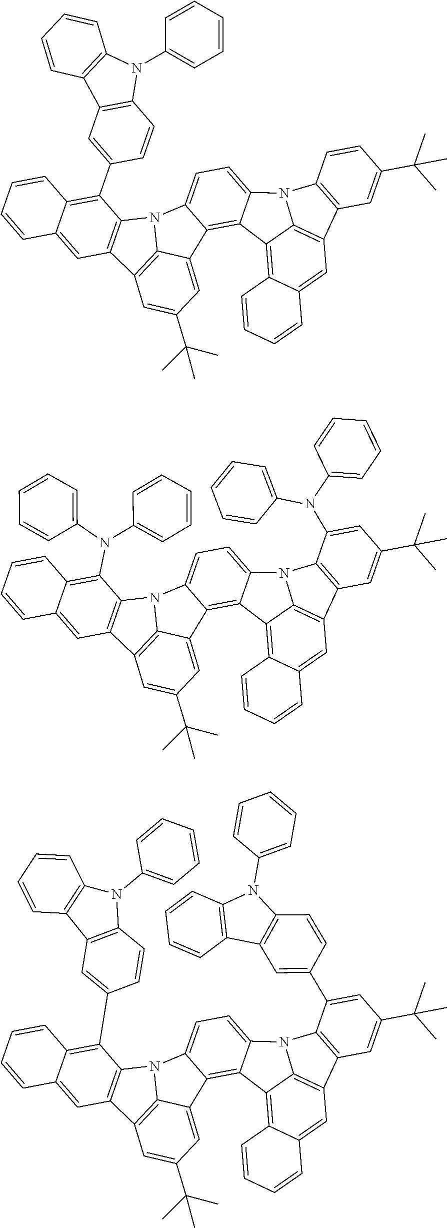 [SMILES: CC(C)(C)c1ccc2c(c1)c1cc3ccccc3c3c4c5c6cc(C(C)(C)C)cc7c8cc9ccccc9c(-c9ccc%10c(c9)c9ccccc9n%10-c9ccccc9)c8n(c5ccc4n2c13)c76 ; CC(C)(C)c1cc(N(c2ccccc2)c2ccccc2)c2c(c1)c1cc3ccccc3c3c4c5c6cc(C(C)(C)C)cc7c8cc9ccccc9c(N(c9ccccc9)c9ccccc9)c8n(c5ccc4n2c13)c76 ; CC(C)(C)c1cc(-c2ccc3c(c2)c2ccccc2n3-c2ccccc2)c2c(c1)c1cc3ccccc3c3c4c5c6cc(C(C)(C)C)cc7c8cc9ccccc9c(-c9ccc%10c(c9)c9ccccc9n%10-c9ccccc9)c8n(c5ccc4n2c13)c76]

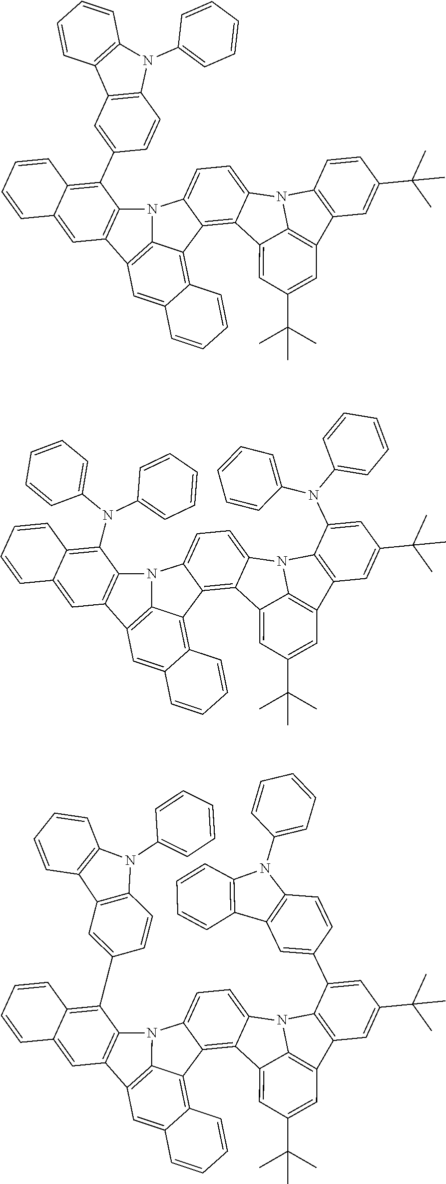 [SMILES: CC(C)(C)c1ccc2c(c1)c1cc(C(C)(C)C)cc3c4c5c6c7ccccc7cc7c8cc9ccccc9c(-c9ccc%10c(c9)c9ccccc9n%10-c9ccccc9)c8n(c5ccc4n2c13)c76 ; CC(C)(C)c1cc(N(c2ccccc2)c2ccccc2)c2c(c1)c1cc(C(C)(C)C)cc3c4c5c6c7ccccc7cc7c8cc9ccccc9c(N(c9ccccc9)c9ccccc9)c8n(c5ccc4n2c13)c76 ; CC(C)(C)c1cc(-c2ccc3c(c2)c2ccccc2n3-c2ccccc2)c2c(c1)c1cc(C(C)(C)C)cc3c4c5c6c7ccccc7cc7c8cc9ccccc9c(-c9ccc%10c(c9)c9ccccc9n%10-c9ccccc9)c8n(c5ccc4n2c13)c76]

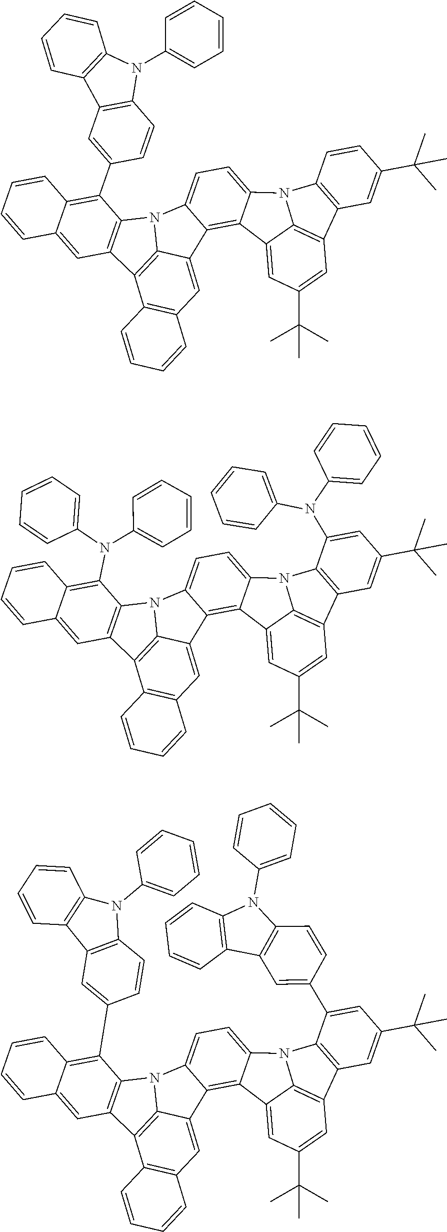 [SMILES: CC(C)(C)c1ccc2c(c1)c1cc(C(C)(C)C)cc3c4c5c6cc7ccccc7c7c8cc9ccccc9c(-c9ccc%10c(c9)c9ccccc9n%10-c9ccccc9)c8n(c5ccc4n2c13)c67 ; CC(C)(C)c1cc(N(c2ccccc2)c2ccccc2)c2c(c1)c1cc(C(C)(C)C)cc3c4c5c6cc7ccccc7c7c8cc9ccccc9c(N(c9ccccc9)c9ccccc9)c8n(c5ccc4n2c13)c67 ; CC(C)(C)c1cc(-c2ccc3c(c2)c2ccccc2n3-c2ccccc2)c2c(c1)c1cc(C(C)(C)C)cc3c4c5c6cc7ccccc7c7c8cc9ccccc9c(-c9ccc%10c(c9)c9ccccc9n%10-c9ccccc9)c8n(c5ccc4n2c13)c67]

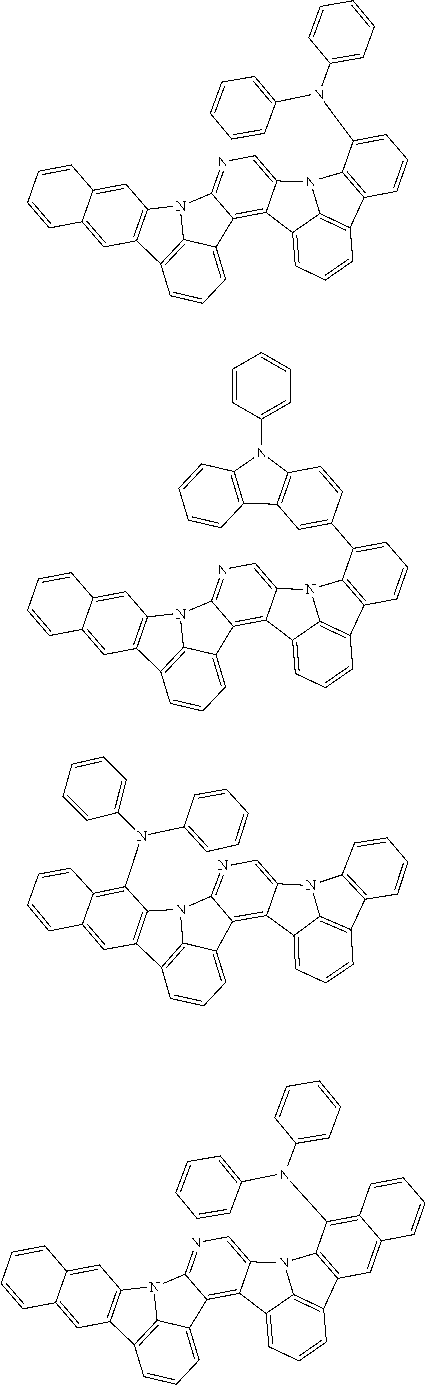 [SMILES: c1ccc(N(c2ccccc2)c2cccc3c4cccc5c6c7c8cccc9c%10cc%11ccccc%11cc%10n(c7ncc6n(c23)c45)c98)cc1 ; c1ccc(-n2c3ccccc3c3cc(-c4cccc5c6cccc7c8c9c%10cccc%11c%12cc%13ccccc%13cc%12n(c9ncc8n(c45)c67)c%11%10)ccc32)cc1 ; c1ccc(N(c2ccccc2)c2c3ccccc3cc3c4cccc5c6c7c8cccc9c%10ccccc%10n(c7cnc6n(c23)c45)c98)cc1 ; c1ccc(N(c2ccccc2)c2c3ccccc3cc3c4cccc5c6c7c8cccc9c%10cc%11ccccc%11cc%10n(c7ncc6n(c23)c45)c98)cc1]

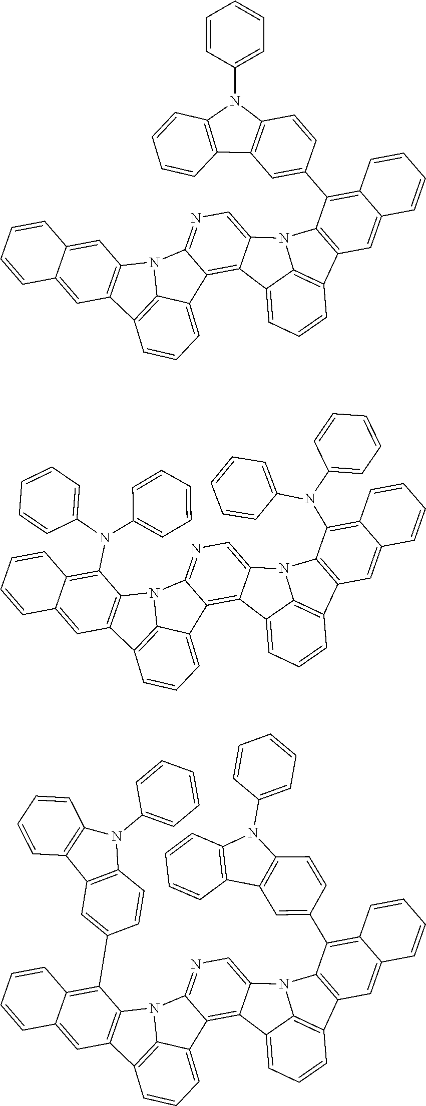 [SMILES: c1ccc(-n2c3ccccc3c3cc(-c4c5ccccc5cc5c6cccc7c8c9c%10cccc%11c%12cc%13ccccc%13cc%12n(c9ncc8n(c45)c67)c%11%10)ccc32)cc1 ; c1ccc(N(c2ccccc2)c2c3ccccc3cc3c4cccc5c6c7c8cccc9c%10cc%11ccccc%11c(N(c%11ccccc%11)c%11ccccc%11)c%10n(c7ncc6n(c23)c45)c98)cc1 ; c1ccc(-n2c3ccccc3c3cc(-c4c5ccccc5cc5c6cccc7c8c9c%10cccc%11c%12cc%13ccccc%13c(-c%13ccc%14c(c%13)c%13ccccc%13n%14-c%13ccccc%13)c%12n(c9ncc8n(c45)c67)c%11%10)ccc32)cc1]

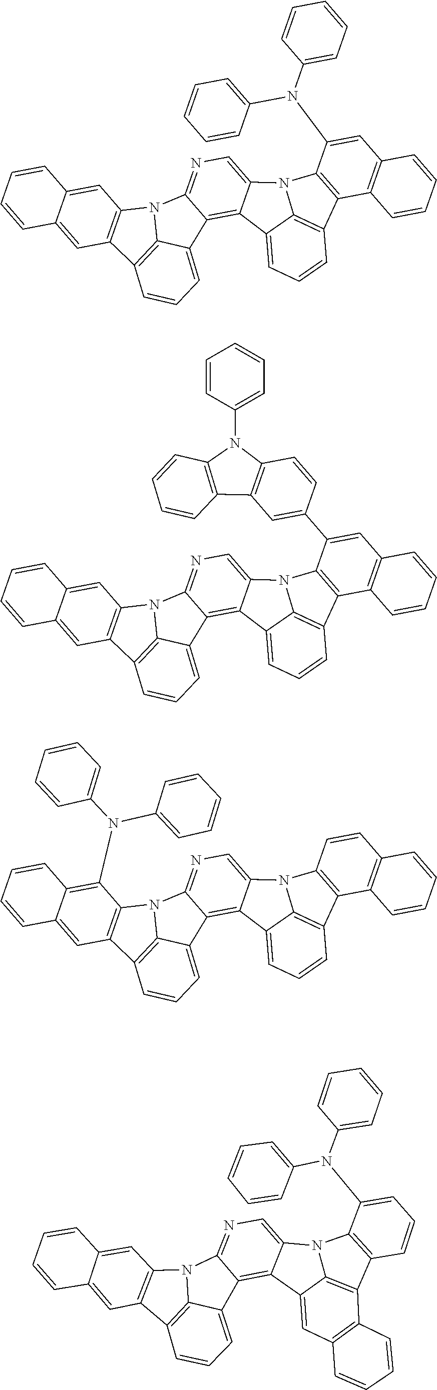 [SMILES: c1ccc(N(c2ccccc2)c2cc3ccccc3c3c4cccc5c6c7c8cccc9c%10cc%11ccccc%11cc%10n(c7ncc6n(c23)c54)c98)cc1 ; c1ccc(-n2c3ccccc3c3cc(-c4cc5ccccc5c5c6cccc7c8c9c%10cccc%11c%12cc%13ccccc%13cc%12n(c9ncc8n(c45)c76)c%11%10)ccc32)cc1 ; c1ccc(N(c2ccccc2)c2c3ccccc3cc3c4cccc5c6c7c8cccc9c%10c%11ccccc%11ccc%10n(c7cnc6n(c23)c45)c98)cc1 ; c1ccc(N(c2ccccc2)c2cccc3c4c5ccccc5cc5c6c7c8cccc9c%10cc%11ccccc%11cc%10n(c7ncc6n(c23)c54)c98)cc1]

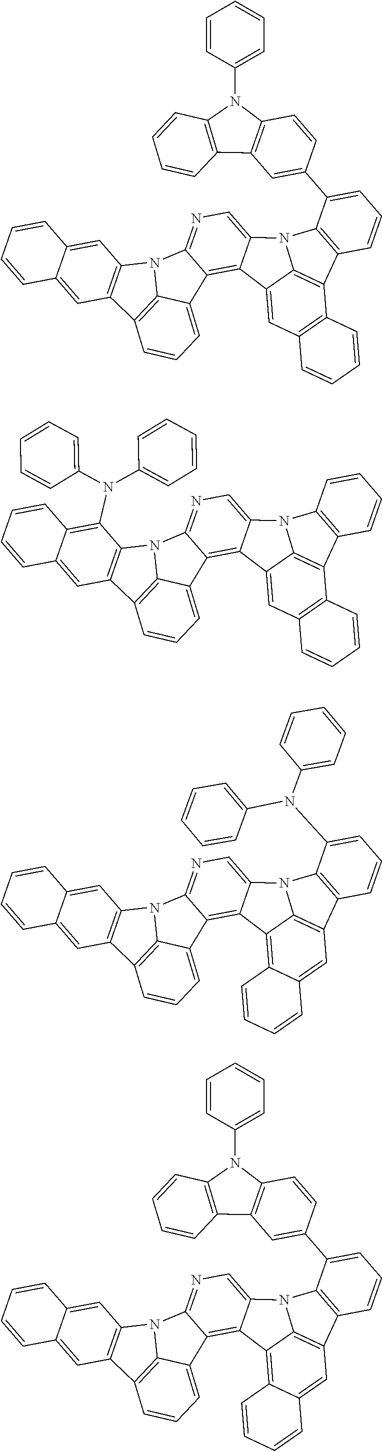

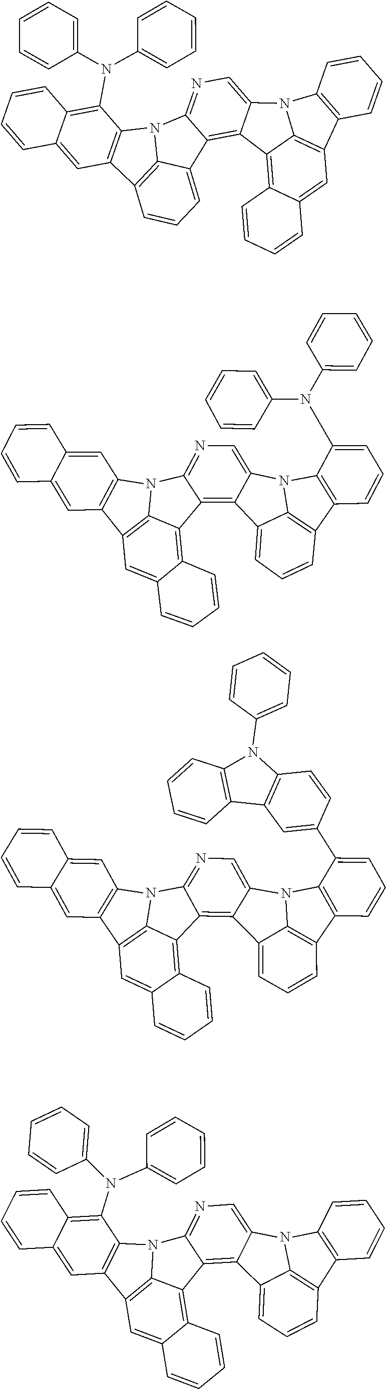 [SMILES: c1ccc(N(c2ccccc2)c2c3ccccc3cc3c4cccc5c6c7c8c9ccccc9cc9c%10ccccc%10n(c7cnc6n(c23)c45)c98)cc1 ; c1ccc(N(c2ccccc2)c2cccc3c4cccc5c6c7c8c9ccccc9cc9c%10cc%11ccccc%11cc%10n(c7ncc6n(c23)c45)c98)cc1 ; c1ccc(-n2c3ccccc3c3cc(-c4cccc5c6cccc7c8c9c%10c%11ccccc%11cc%11c%12cc%13ccccc%13cc%12n(c9ncc8n(c45)c67)c%11%10)ccc32)cc1 ; c1ccc(N(c2ccccc2)c2c3ccccc3cc3c4cc5ccccc5c5c6c7c8cccc9c%10ccccc%10n(c7cnc6n(c23)c45)c98)cc1]

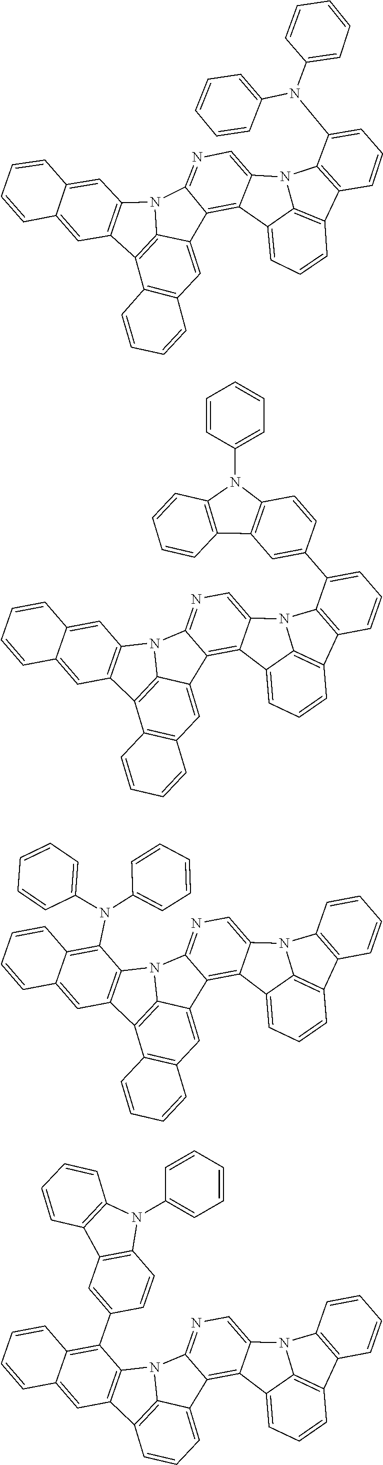

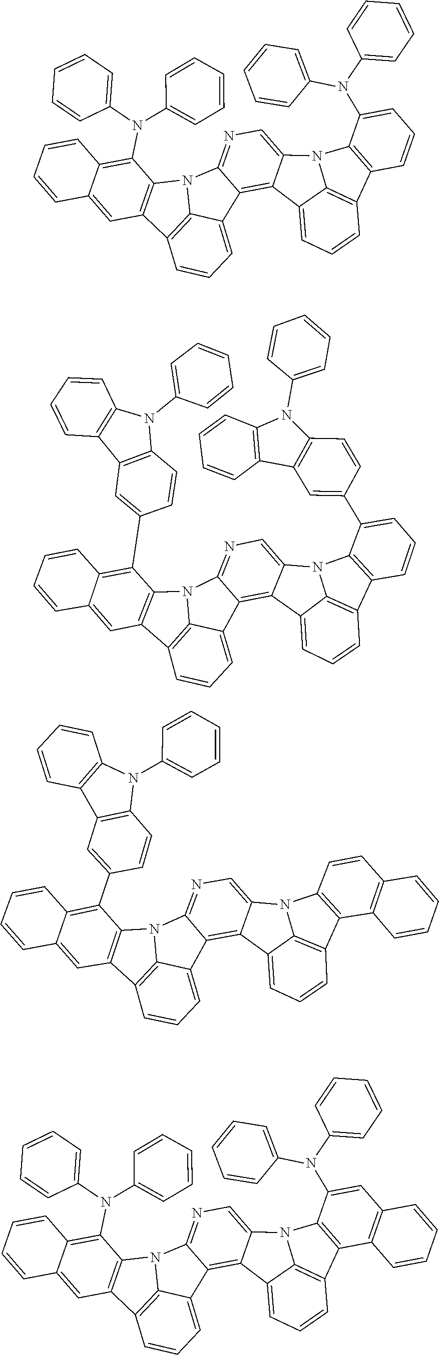

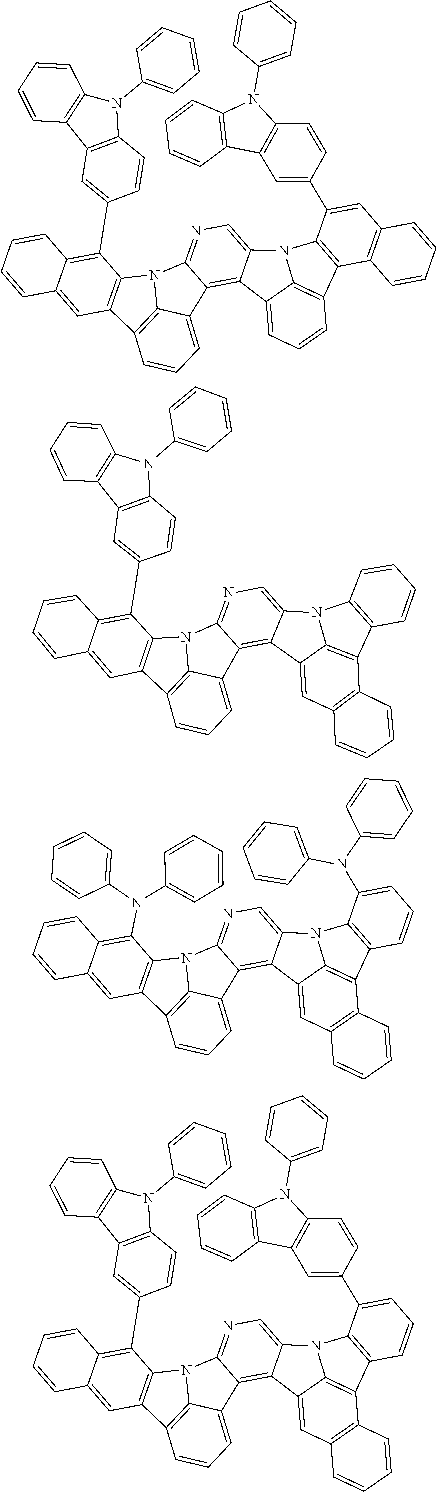

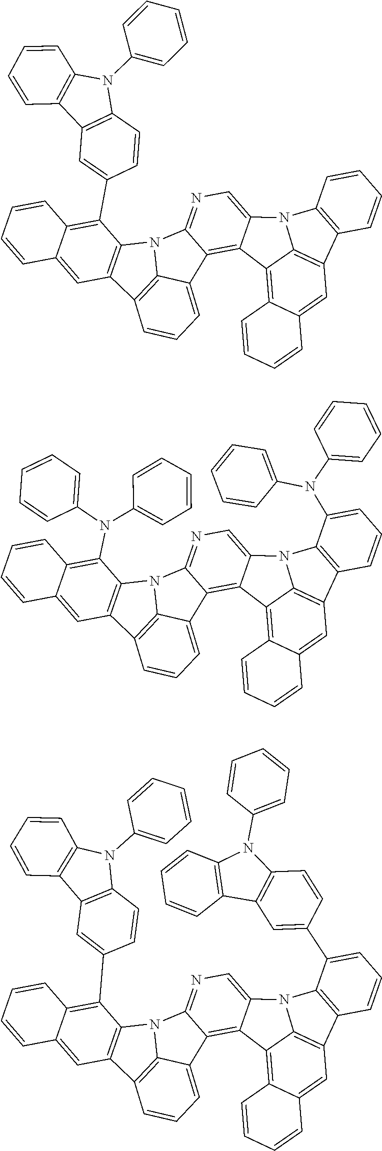

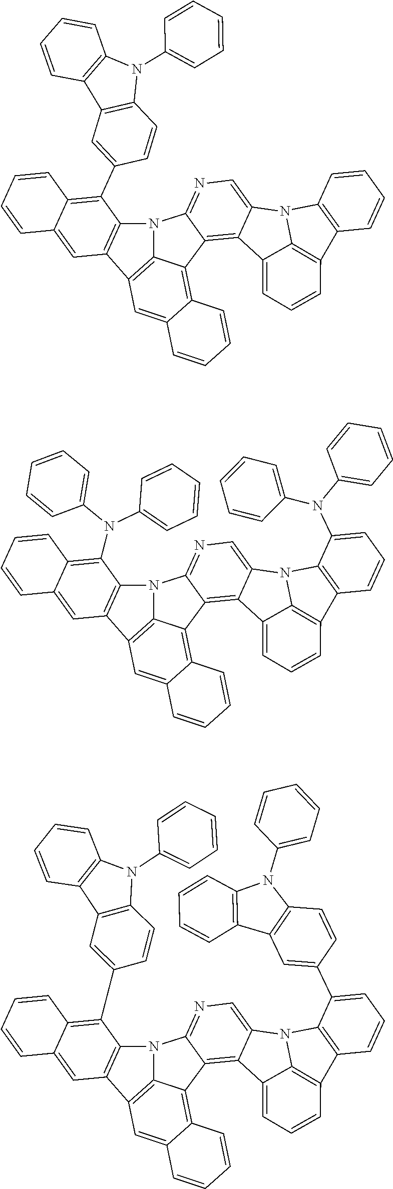 [SMILES: c1ccc(-n2c3ccccc3c3cc(-c4c5ccccc5cc5c6cc7ccccc7c7c8c9c%10cccc%11c%12ccccc%12n(c9cnc8n(c45)c67)c%11%10)ccc32)cc1 ; c1ccc(N(c2ccccc2)c2cccc3c4cccc5c6c7c8c9ccccc9cc9c%10cc%11ccccc%11c(N(c%11ccccc%11)c%11ccccc%11)c%10n(c7ncc6n(c23)c45)c98)cc1 ; c1ccc(-n2c3ccccc3c3cc(-c4cccc5c6cccc7c8c9c%10c%11ccccc%11cc%11c%12cc%13ccccc%13c(-c%13ccc%14c(c%13)c%13ccccc%13n%14-c%13ccccc%13)c%12n(c9ncc8n(c45)c67)c%11%10)ccc32)cc1]

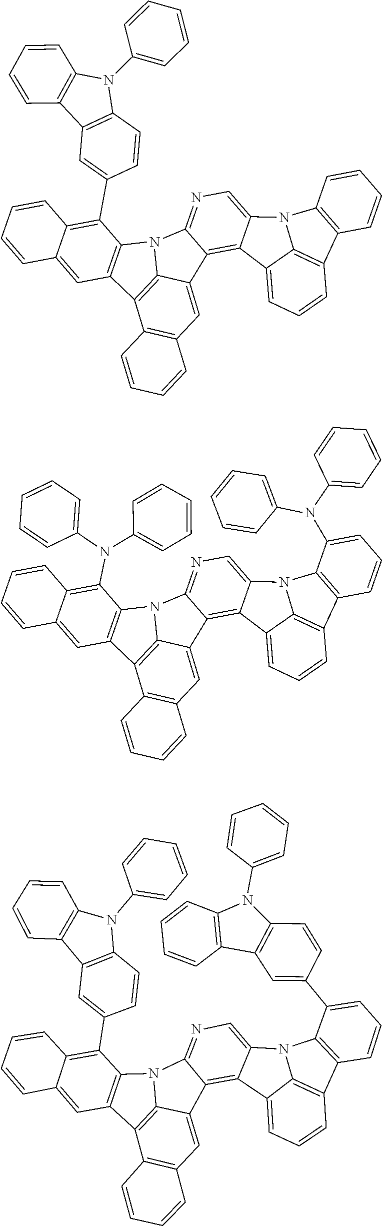 [SMILES: c1ccc(-n2c3ccccc3c3cc(-c4c5ccccc5cc5c6c7ccccc7cc7c8c9c%10cccc%11c%12ccccc%12n(c9cnc8n(c45)c76)c%11%10)ccc32)cc1 ; c1ccc(N(c2ccccc2)c2cccc3c4cccc5c6c7c8cc9ccccc9c9c%10cc%11ccccc%11c(N(c%11ccccc%11)c%11ccccc%11)c%10n(c7ncc6n(c23)c45)c89)cc1 ; c1ccc(-n2c3ccccc3c3cc(-c4cccc5c6cccc7c8c9c%10cc%11ccccc%11c%11c%12cc%13ccccc%13c(-c%13ccc%14c(c%13)c%13ccccc%13n%14-c%13ccccc%13)c%12n(c9ncc8n(c45)c67)c%10%11)ccc32)cc1]

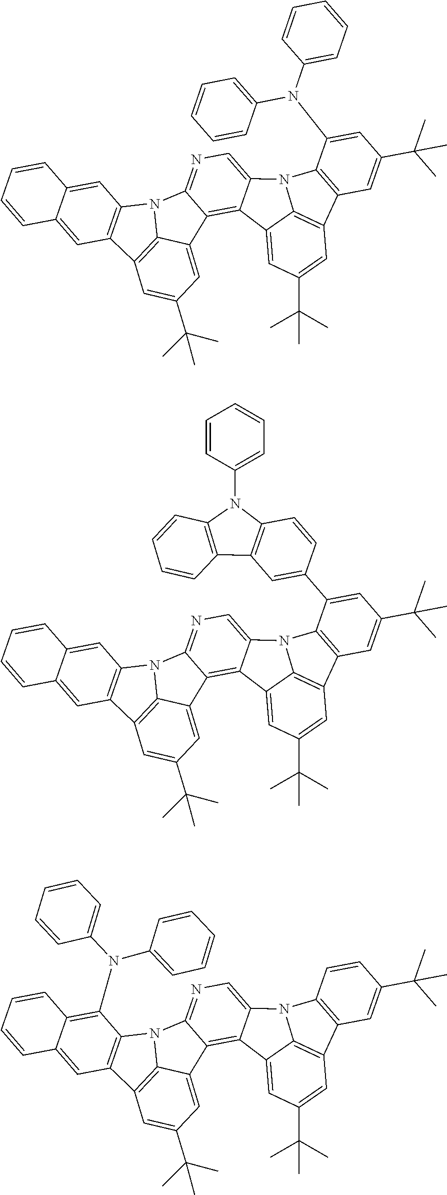 [SMILES: CC(C)(C)c1cc(N(c2ccccc2)c2ccccc2)c2c(c1)c1cc(C(C)(C)C)cc3c4c5c6cc(C(C)(C)C)cc7c8cc9ccccc9cc8n(c5ncc4n2c13)c76 ; CC(C)(C)c1cc(-c2ccc3c(c2)c2ccccc2n3-c2ccccc2)c2c(c1)c1cc(C(C)(C)C)cc3c4c5c6cc(C(C)(C)C)cc7c8cc9ccccc9cc8n(c5ncc4n2c13)c76 ; CC(C)(C)c1ccc2c(c1)c1cc(C(C)(C)C)cc3c4c5c6cc(C(C)(C)C)cc7c8cc9ccccc9c(N(c9ccccc9)c9ccccc9)c8n(c5ncc4n2c13)c76]

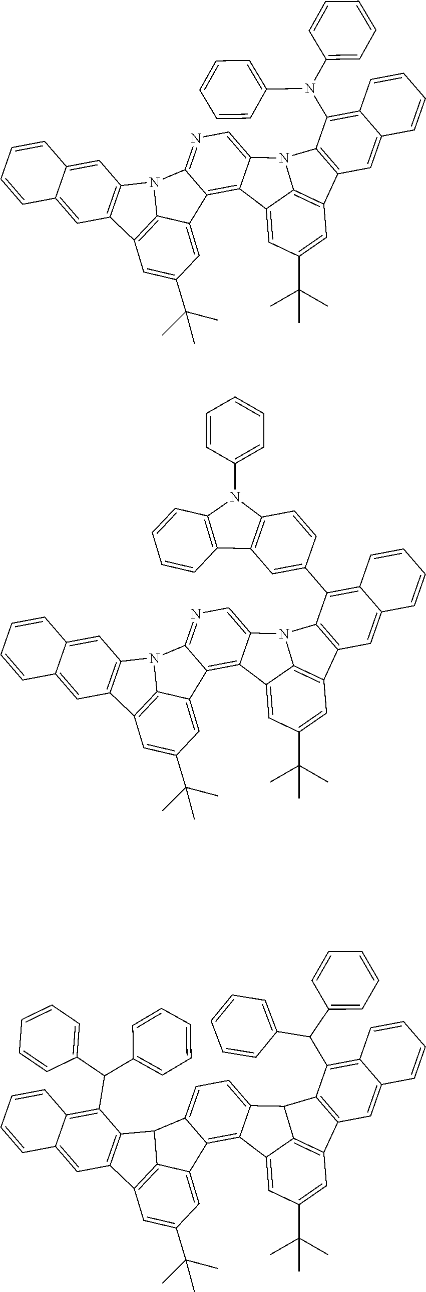

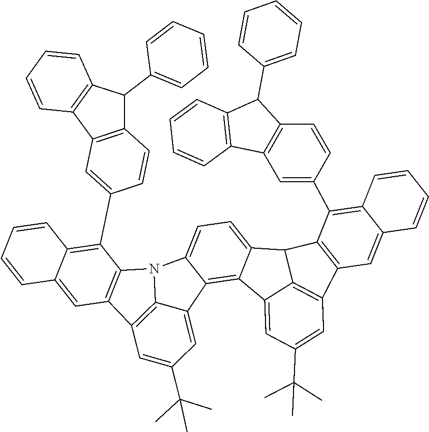 [SMILES: CC(C)(C)c1cc2c3c(c1)-c1c(ccc4c1c1cc(C(C)(C)C)cc5c6cc7ccccc7c(-c7ccc8c(c7)-c7ccccc7C8c7ccccc7)c6n4c51)C3c1c-2cc2ccccc2c1-c1ccc2c(c1)-c1ccccc1C2c1ccccc1]

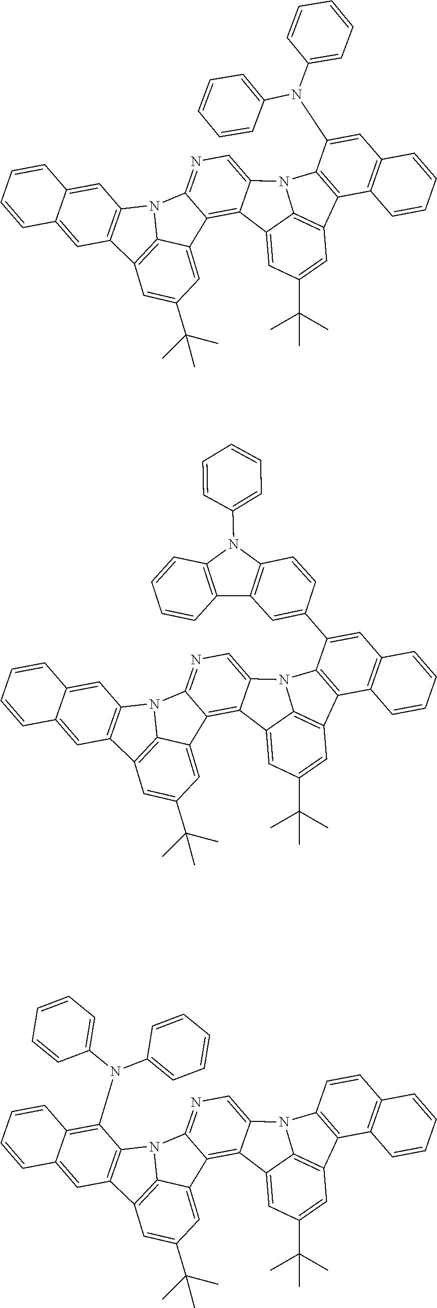 [SMILES: CC(C)(C)c1cc2c3cc4ccccc4cc3n3c4ncc5c(c6cc(C(C)(C)C)cc7c8c9ccccc9cc(N(c9ccccc9)c9ccccc9)c8n5c67)c4c(c1)c23 ; CC(C)(C)c1cc2c3cc4ccccc4cc3n3c4ncc5c(c6cc(C(C)(C)C)cc7c8c9ccccc9cc(-c9ccc%10c(c9)c9ccccc9n%10-c9ccccc9)c8n5c67)c4c(c1)c23 ; CC(C)(C)c1cc2c3c4ccccc4ccc3n3c4cnc5c(c6cc(C(C)(C)C)cc7c8cc9ccccc9c(N(c9ccccc9)c9ccccc9)c8n5c76)c4c(c1)c23]

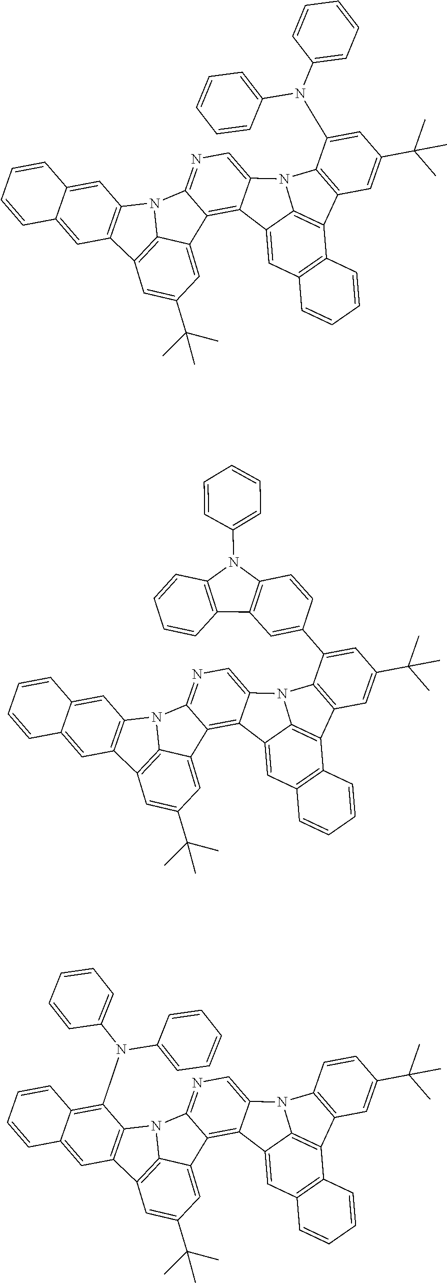 [SMILES: CC(C)(C)c1cc(N(c2ccccc2)c2ccccc2)c2c(c1)c1c3ccccc3cc3c4c5c6cc(C(C)(C)C)cc7c8cc9ccccc9cc8n(c5ncc4n2c31)c76 ; CC(C)(C)c1cc(-c2ccc3c(c2)c2ccccc2n3-c2ccccc2)c2c(c1)c1c3ccccc3cc3c4c5c6cc(C(C)(C)C)cc7c8cc9ccccc9cc8n(c5ncc4n2c31)c76 ; CC(C)(C)c1ccc2c(c1)c1c3ccccc3cc3c4c5c6cc(C(C)(C)C)cc7c8cc9ccccc9c(N(c9ccccc9)c9ccccc9)c8n(c5ncc4n2c31)c76]

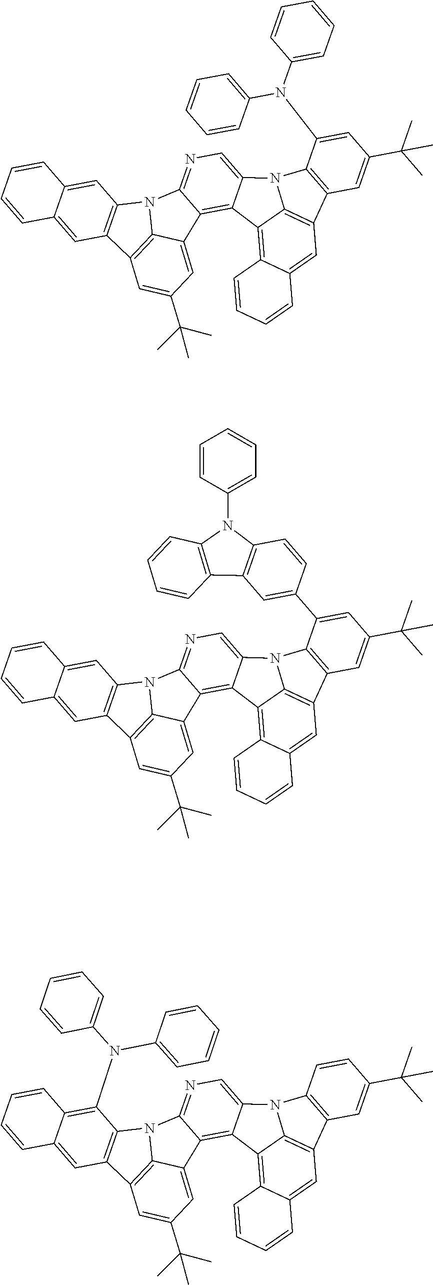 [SMILES: CC(C)(C)c1cc(N(c2ccccc2)c2ccccc2)c2c(c1)c1cc3ccccc3c3c4c5c6cc(C(C)(C)C)cc7c8cc9ccccc9cc8n(c5ncc4n2c13)c76 ; CC(C)(C)c1cc(-c2ccc3c(c2)c2ccccc2n3-c2ccccc2)c2c(c1)c1cc3ccccc3c3c4c5c6cc(C(C)(C)C)cc7c8cc9ccccc9cc8n(c5ncc4n2c13)c76 ; CC(C)(C)c1ccc2c(c1)c1cc3ccccc3c3c4c5c6cc(C(C)(C)C)cc7c8cc9ccccc9c(N(c9ccccc9)c9ccccc9)c8n(c5ncc4n2c13)c76]

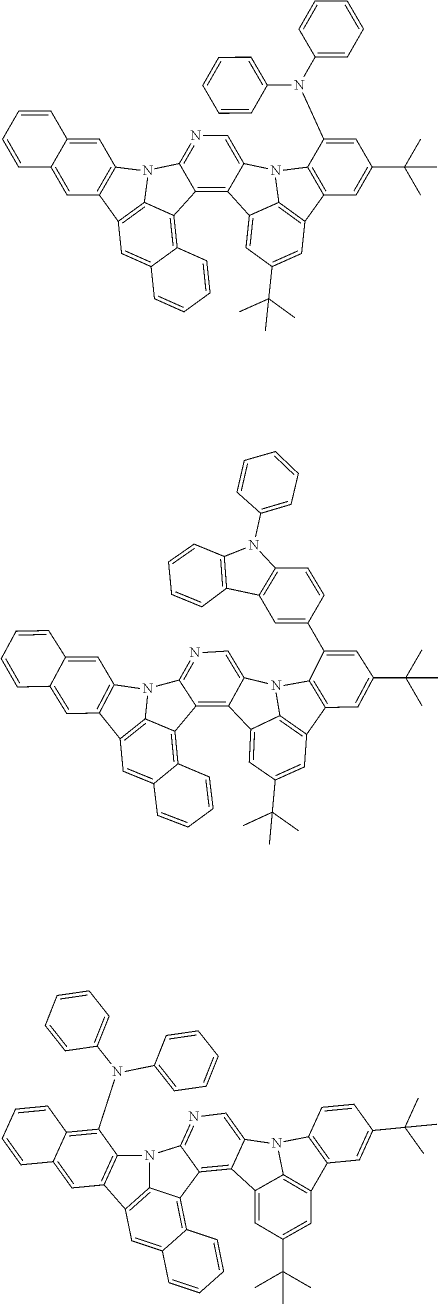 [SMILES: CC(C)(C)c1cc(N(c2ccccc2)c2ccccc2)c2c(c1)c1cc(C(C)(C)C)cc3c4c5c6c7ccccc7cc7c8cc9ccccc9cc8n(c5ncc4n2c13)c76 ; CC(C)(C)c1cc(-c2ccc3c(c2)c2ccccc2n3-c2ccccc2)c2c(c1)c1cc(C(C)(C)C)cc3c4c5c6c7ccccc7cc7c8cc9ccccc9cc8n(c5ncc4n2c13)c76 ; CC(C)(C)c1ccc2c(c1)c1cc(C(C)(C)C)cc3c4c5c6c7ccccc7cc7c8cc9ccccc9c(N(c9ccccc9)c9ccccc9)c8n(c5ncc4n2c13)c76]

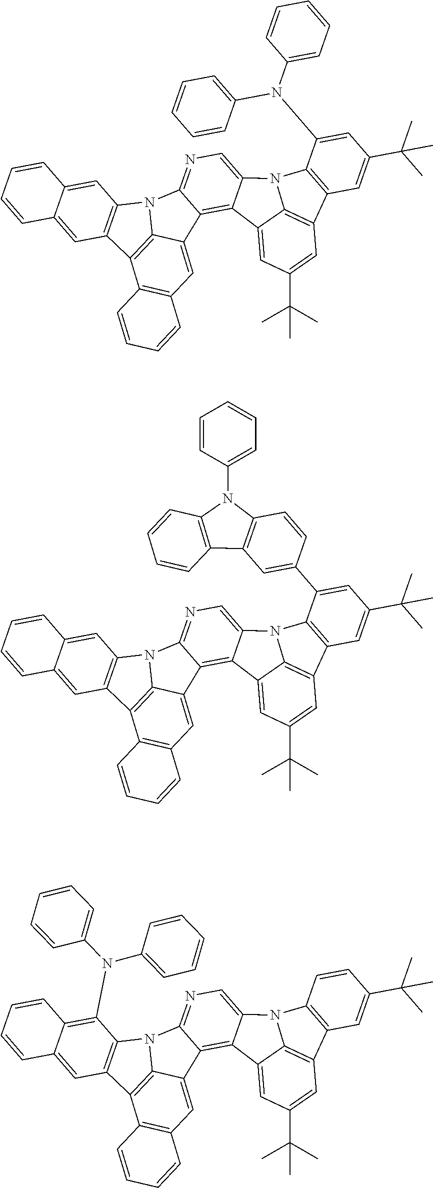 [SMILES: CC(C)(C)c1cc(N(c2ccccc2)c2ccccc2)c2c(c1)c1cc(C(C)(C)C)cc3c4c5c6cc7ccccc7c7c8cc9ccccc9cc8n(c5ncc4n2c13)c67 ; CC(C)(C)c1cc(-c2ccc3c(c2)c2ccccc2n3-c2ccccc2)c2c(c1)c1cc(C(C)(C)C)cc3c4c5c6cc7ccccc7c7c8cc9ccccc9cc8n(c5ncc4n2c13)c67 ; CC(C)(C)c1ccc2c(c1)c1cc(C(C)(C)C)cc3c4c5c6cc7ccccc7c7c8cc9ccccc9c(N(c9ccccc9)c9ccccc9)c8n(c5ncc4n2c13)c67]

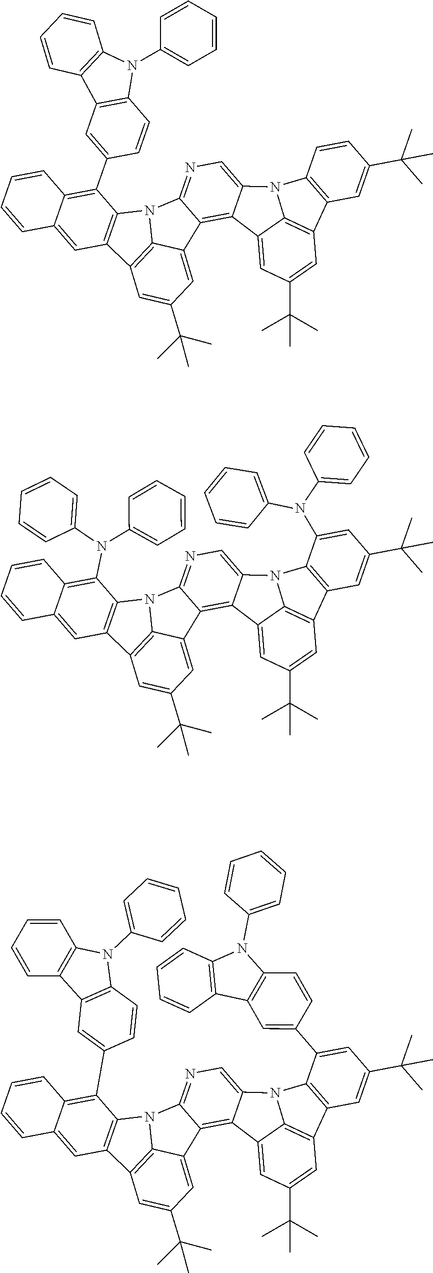 [SMILES: CC(C)(C)c1ccc2c(c1)c1cc(C(C)(C)C)cc3c4c5c6cc(C(C)(C)C)cc7c8cc9ccccc9c(-c9ccc%10c(c9)c9ccccc9n%10-c9ccccc9)c8n(c5ncc4n2c13)c76 ; CC(C)(C)c1cc(N(c2ccccc2)c2ccccc2)c2c(c1)c1cc(C(C)(C)C)cc3c4c5c6cc(C(C)(C)C)cc7c8cc9ccccc9c(N(c9ccccc9)c9ccccc9)c8n(c5ncc4n2c13)c76 ; CC(C)(C)c1cc(-c2ccc3c(c2)c2ccccc2n3-c2ccccc2)c2c(c1)c1cc(C(C)(C)C)cc3c4c5c6cc(C(C)(C)C)cc7c8cc9ccccc9c(-c9ccc%10c(c9)c9ccccc9n%10-c9ccccc9)c8n(c5ncc4n2c13)c76]

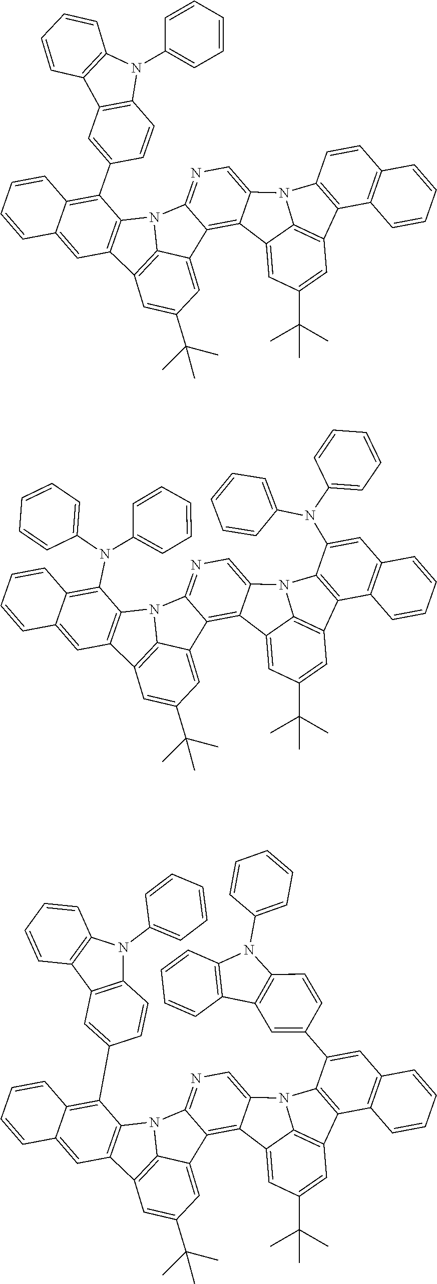 [SMILES: CC(C)(C)c1cc2c3c4ccccc4ccc3n3c4cnc5c(c6cc(C(C)(C)C)cc7c8cc9ccccc9c(-c9ccc%10c(c9)c9ccccc9n%10-c9ccccc9)c8n5c76)c4c(c1)c23 ; CC(C)(C)c1cc2c3c4c5cc(C(C)(C)C)cc6c7cc8ccccc8c(N(c8ccccc8)c8ccccc8)c7n(c4ncc3n3c4c(N(c7ccccc7)c7ccccc7)cc7ccccc7c4c(c1)c23)c65 ; CC(C)(C)c1cc2c3c4c5cc(C(C)(C)C)cc6c7cc8ccccc8c(-c8ccc9c(c8)c8ccccc8n9-c8ccccc8)c7n(c4ncc3n3c4c(-c7ccc8c(c7)c7ccccc7n8-c7ccccc7)cc7ccccc7c4c(c1)c23)c65]

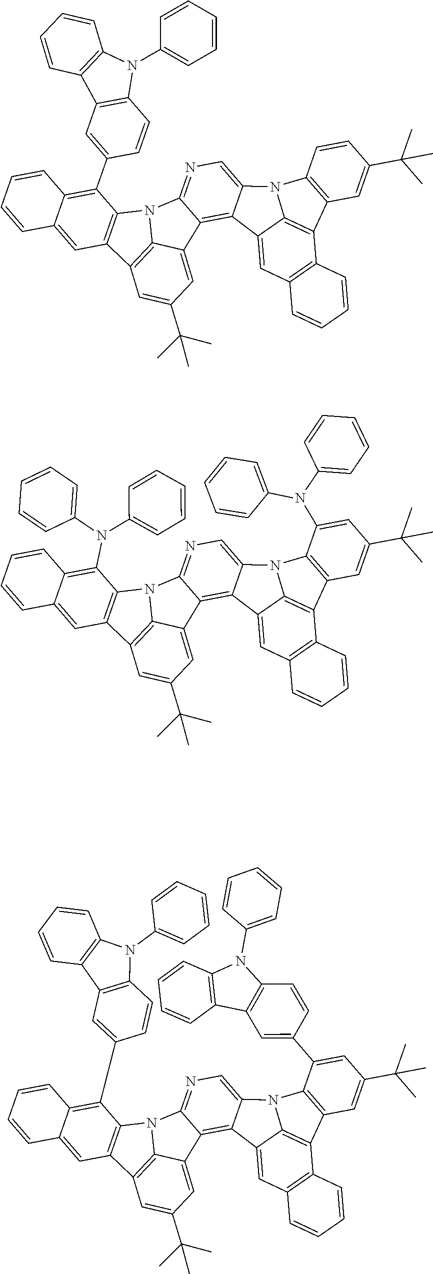 [SMILES: CC(C)(C)c1ccc2c(c1)c1c3ccccc3cc3c4c5c6cc(C(C)(C)C)cc7c8cc9ccccc9c(-c9ccc%10c(c9)c9ccccc9n%10-c9ccccc9)c8n(c5ncc4n2c31)c76 ; CC(C)(C)c1cc(N(c2ccccc2)c2ccccc2)c2c(c1)c1c3ccccc3cc3c4c5c6cc(C(C)(C)C)cc7c8cc9ccccc9c(N(c9ccccc9)c9ccccc9)c8n(c5ncc4n2c31)c76 ; CC(C)(C)c1cc(-c2ccc3c(c2)c2ccccc2n3-c2ccccc2)c2c(c1)c1c3ccccc3cc3c4c5c6cc(C(C)(C)C)cc7c8cc9ccccc9c(-c9ccc%10c(c9)c9ccccc9n%10-c9ccccc9)c8n(c5ncc4n2c31)c76]

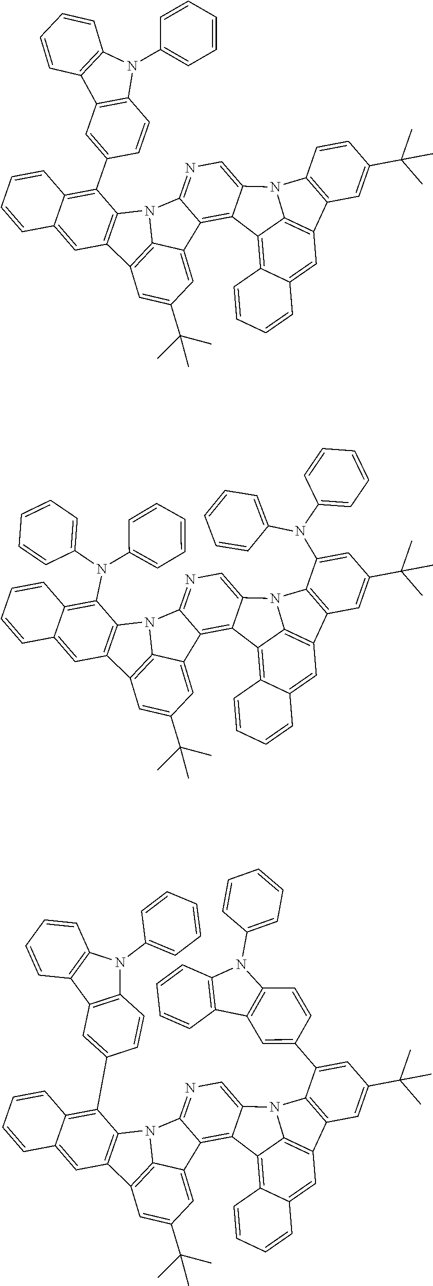 [SMILES: CC(C)(C)c1ccc2c(c1)c1cc3ccccc3c3c4c5c6cc(C(C)(C)C)cc7c8cc9ccccc9c(-c9ccc%10c(c9)c9ccccc9n%10-c9ccccc9)c8n(c5ncc4n2c13)c76 ; CC(C)(C)c1cc(N(c2ccccc2)c2ccccc2)c2c(c1)c1cc3ccccc3c3c4c5c6cc(C(C)(C)C)cc7c8cc9ccccc9c(N(c9ccccc9)c9ccccc9)c8n(c5ncc4n2c13)c76 ; CC(C)(C)c1cc(-c2ccc3c(c2)c2ccccc2n3-c2ccccc2)c2c(c1)c1cc3ccccc3c3c4c5c6cc(C(C)(C)C)cc7c8cc9ccccc9c(-c9ccc%10c(c9)c9ccccc9n%10-c9ccccc9)c8n(c5ncc4n2c13)c76]

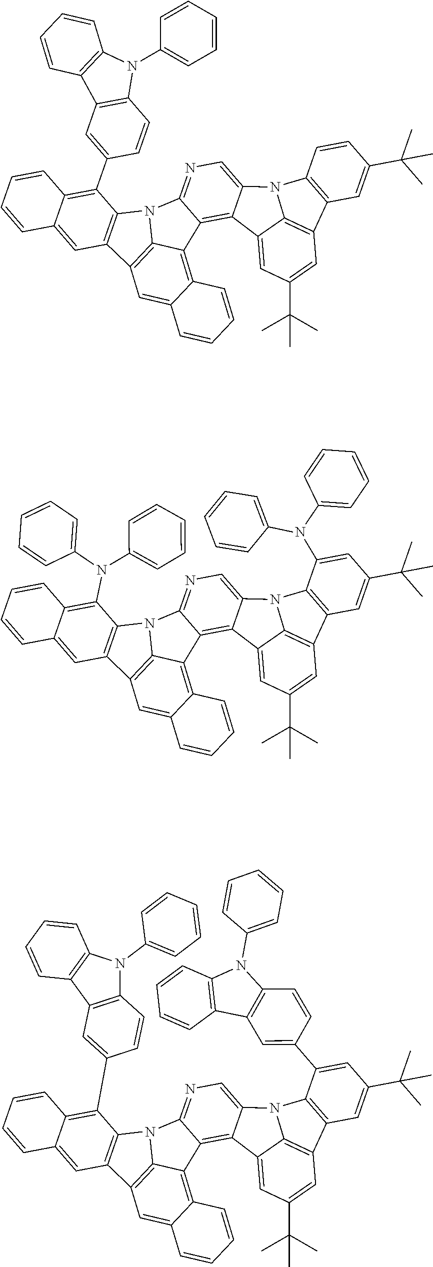 [SMILES: CC(C)(C)c1ccc2c(c1)c1cc(C(C)(C)C)cc3c4c5c6c7ccccc7cc7c8cc9ccccc9c(-c9ccc%10c(c9)c9ccccc9n%10-c9ccccc9)c8n(c5ncc4n2c13)c76 ; CC(C)(C)c1cc(N(c2ccccc2)c2ccccc2)c2c(c1)c1cc(C(C)(C)C)cc3c4c5c6c7ccccc7cc7c8cc9ccccc9c(N(c9ccccc9)c9ccccc9)c8n(c5ncc4n2c13)c76 ; CC(C)(C)c1cc(-c2ccc3c(c2)c2ccccc2n3-c2ccccc2)c2c(c1)c1cc(C(C)(C)C)cc3c4c5c6c7ccccc7cc7c8cc9ccccc9c(-c9ccc%10c(c9)c9ccccc9n%10-c9ccccc9)c8n(c5ncc4n2c13)c76]

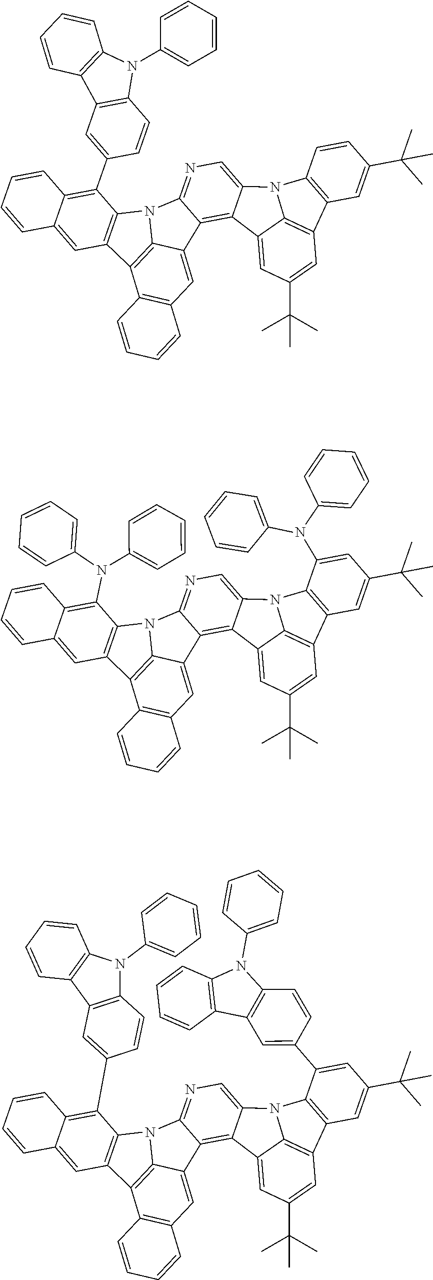 [SMILES: CC(C)(C)c1ccc2c(c1)c1cc(C(C)(C)C)cc3c4c5c6cc7ccccc7c7c8cc9ccccc9c(-c9ccc%10c(c9)c9ccccc9n%10-c9ccccc9)c8n(c5ncc4n2c13)c67 ; CC(C)(C)c1cc(N(c2ccccc2)c2ccccc2)c2c(c1)c1cc(C(C)(C)C)cc3c4c5c6cc7ccccc7c7c8cc9ccccc9c(N(c9ccccc9)c9ccccc9)c8n(c5ncc4n2c13)c67 ; CC(C)(C)c1cc(-c2ccc3c(c2)c2ccccc2n3-c2ccccc2)c2c(c1)c1cc(C(C)(C)C)cc3c4c5c6cc7ccccc7c7c8cc9ccccc9c(-c9ccc%10c(c9)c9ccccc9n%10-c9ccccc9)c8n(c5ncc4n2c13)c67]

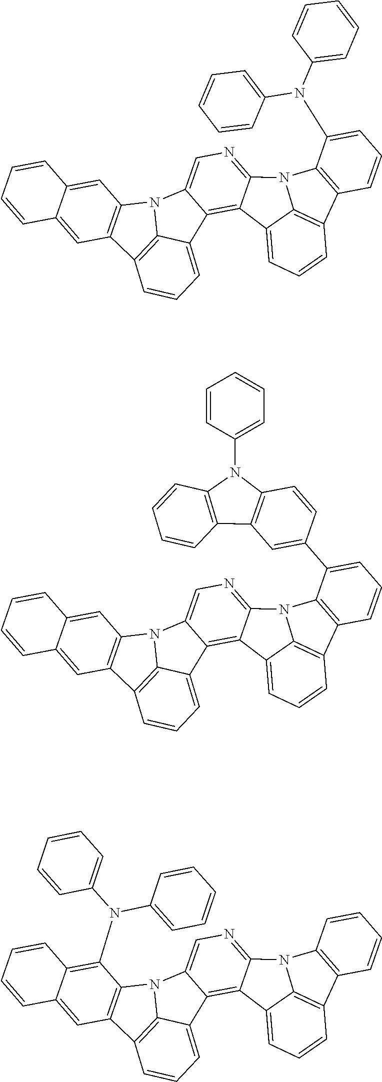 [SMILES: c1ccc(N(c2ccccc2)c2cccc3c4cccc5c6c7c8cccc9c%10cc%11ccccc%11cc%10n(c7cnc6n(c23)c45)c98)cc1 ; c1ccc(-n2c3ccccc3c3cc(-c4cccc5c6cccc7c8c9c%10cccc%11c%12cc%13ccccc%13cc%12n(c9cnc8n(c45)c67)c%11%10)ccc32)cc1 ; c1ccc(N(c2ccccc2)c2c3ccccc3cc3c4cccc5c6c7c8cccc9c%10ccccc%10n(c7ncc6n(c23)c45)c98)cc1]

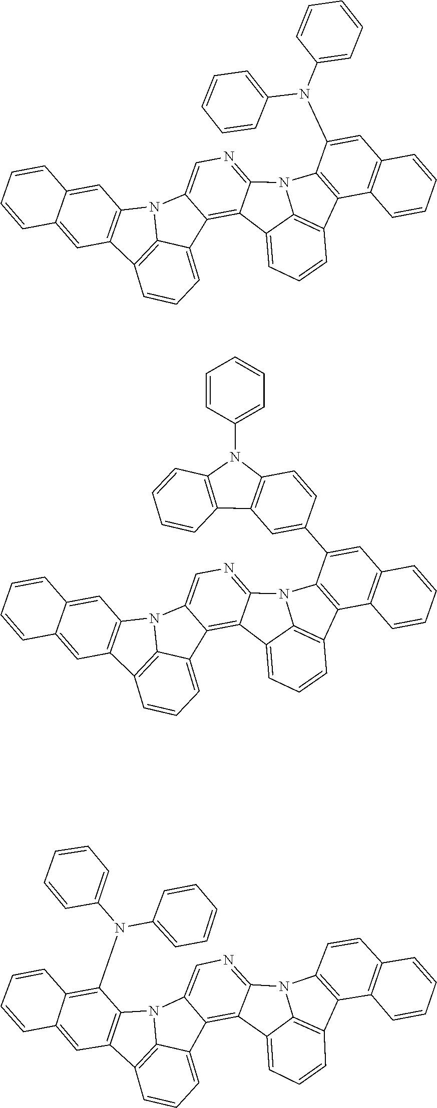 [SMILES: c1ccc(N(c2ccccc2)c2cc3ccccc3c3c4cccc5c6c7c8cccc9c%10cc%11ccccc%11cc%10n(c7cnc6n(c23)c54)c98)cc1 ; c1ccc(-n2c3ccccc3c3cc(-c4cc5ccccc5c5c6cccc7c8c9c%10cccc%11c%12cc%13ccccc%13cc%12n(c9cnc8n(c45)c76)c%11%10)ccc32)cc1 ; c1ccc(N(c2ccccc2)c2c3ccccc3cc3c4cccc5c6c7c8cccc9c%10c%11ccccc%11ccc%10n(c7ncc6n(c23)c45)c98)cc1]

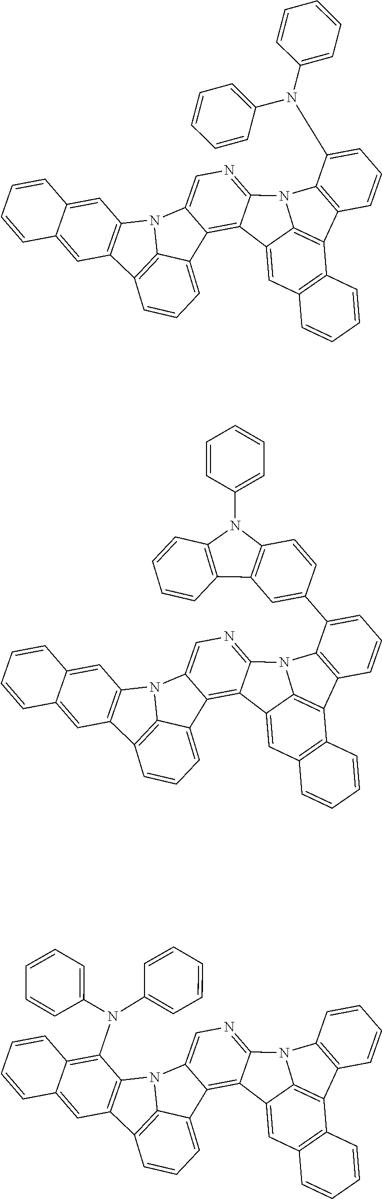 [SMILES: c1ccc(N(c2ccccc2)c2cccc3c4c5ccccc5cc5c6c7c8cccc9c%10cc%11ccccc%11cc%10n(c7cnc6n(c23)c54)c98)cc1 ; c1ccc(-n2c3ccccc3c3cc(-c4cccc5c6c7ccccc7cc7c8c9c%10cccc%11c%12cc%13ccccc%13cc%12n(c9cnc8n(c45)c76)c%11%10)ccc32)cc1 ; c1ccc(N(c2ccccc2)c2c3ccccc3cc3c4cccc5c6c7c8cc9ccccc9c9c%10ccccc%10n(c7ncc6n(c23)c45)c89)cc1]

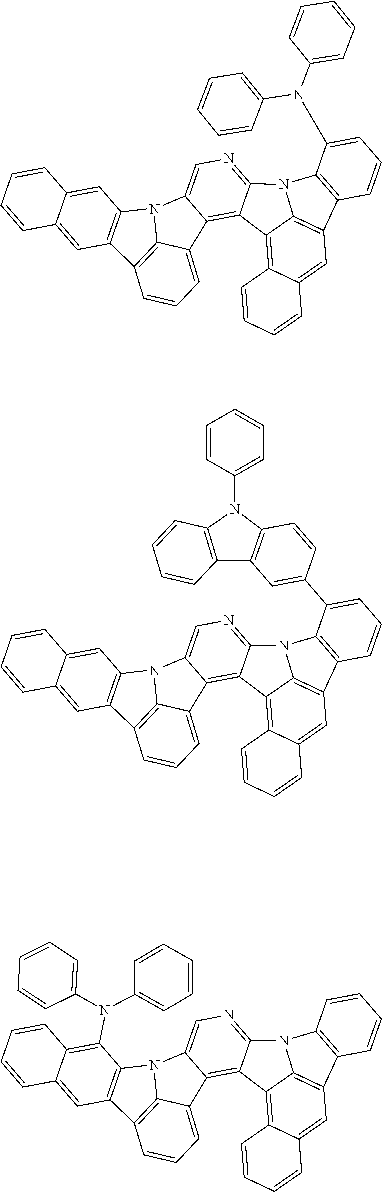 [SMILES: c1ccc(N(c2ccccc2)c2cccc3c4cc5ccccc5c5c6c7c8cccc9c%10cc%11ccccc%11cc%10n(c7cnc6n(c23)c45)c98)cc1 ; c1ccc(-n2c3ccccc3c3cc(-c4cccc5c6cc7ccccc7c7c8c9c%10cccc%11c%12cc%13ccccc%13cc%12n(c9cnc8n(c45)c67)c%11%10)ccc32)cc1 ; c1ccc(N(c2ccccc2)c2c3ccccc3cc3c4cccc5c6c7c8c9ccccc9cc9c%10ccccc%10n(c7ncc6n(c23)c45)c98)cc1]

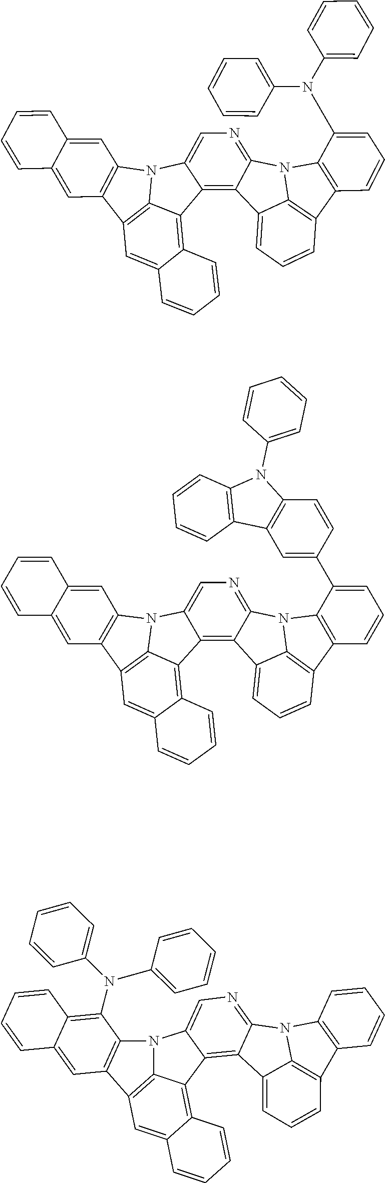 [SMILES: c1ccc(N(c2ccccc2)c2cccc3c4cccc5c6c7c8c9ccccc9cc9c%10cc%11ccccc%11cc%10n(c7cnc6n(c23)c45)c98)cc1 ; c1ccc(-n2c3ccccc3c3cc(-c4cccc5c6cccc7c8c9c%10c%11ccccc%11cc%11c%12cc%13ccccc%13cc%12n(c9cnc8n(c45)c67)c%11%10)ccc32)cc1 ; c1ccc(N(c2ccccc2)c2c3ccccc3cc3c4cc5ccccc5c5c6c7c8cccc9c%10ccccc%10n(c7ncc6n(c23)c45)c98)cc1]

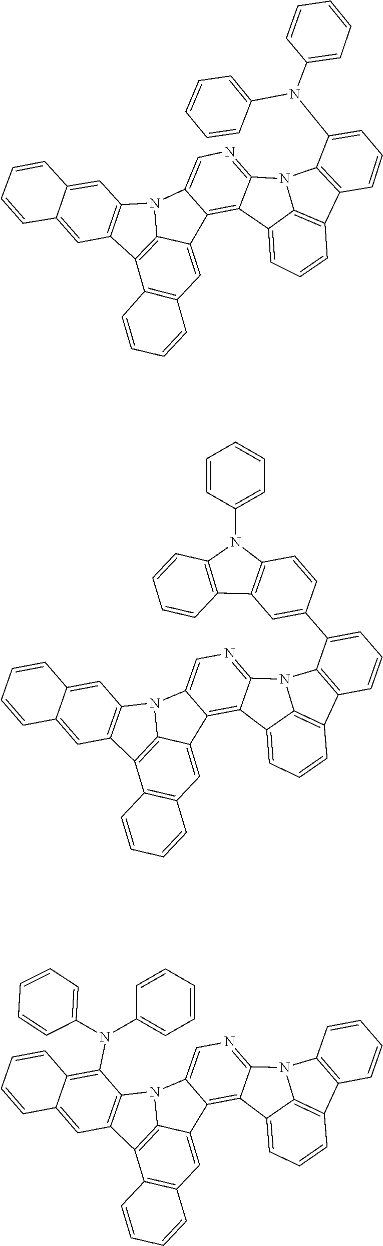 [SMILES: c1ccc(N(c2ccccc2)c2cccc3c4cccc5c6c7c8cc9ccccc9c9c%10cc%11ccccc%11cc%10n(c7cnc6n(c23)c45)c89)cc1 ; c1ccc(-n2c3ccccc3c3cc(-c4cccc5c6cccc7c8c9c%10cc%11ccccc%11c%11c%12cc%13ccccc%13cc%12n(c9cnc8n(c45)c67)c%10%11)ccc32)cc1 ; c1ccc(N(c2ccccc2)c2c3ccccc3cc3c4c5ccccc5cc5c6c7c8cccc9c%10ccccc%10n(c7ncc6n(c23)c54)c98)cc1]

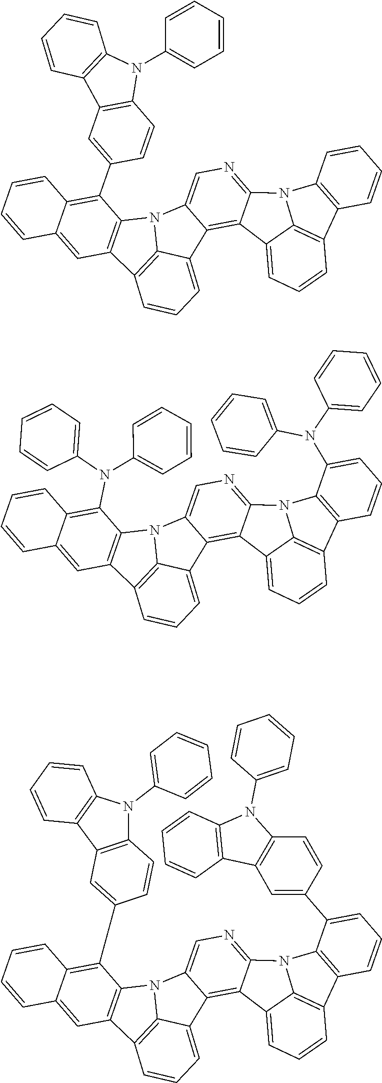 [SMILES: c1ccc(-n2c3ccccc3c3cc(-c4c5ccccc5cc5c6cccc7c8c9c%10cccc%11c%12ccccc%12n(c9ncc8n(c45)c67)c%11%10)ccc32)cc1 ; c1ccc(N(c2ccccc2)c2c3ccccc3cc3c4cccc5c6c7c8cccc9c%10cccc(N(c%11ccccc%11)c%11ccccc%11)c%10n(c7ncc6n(c23)c45)c98)cc1 ; c1ccc(-n2c3ccccc3c3cc(-c4c5ccccc5cc5c6cccc7c8c9c%10cccc%11c%12cccc(-c%13ccc%14c(c%13)c%13ccccc%13n%14-c%13ccccc%13)c%12n(c9ncc8n(c45)c67)c%11%10)ccc32)cc1]

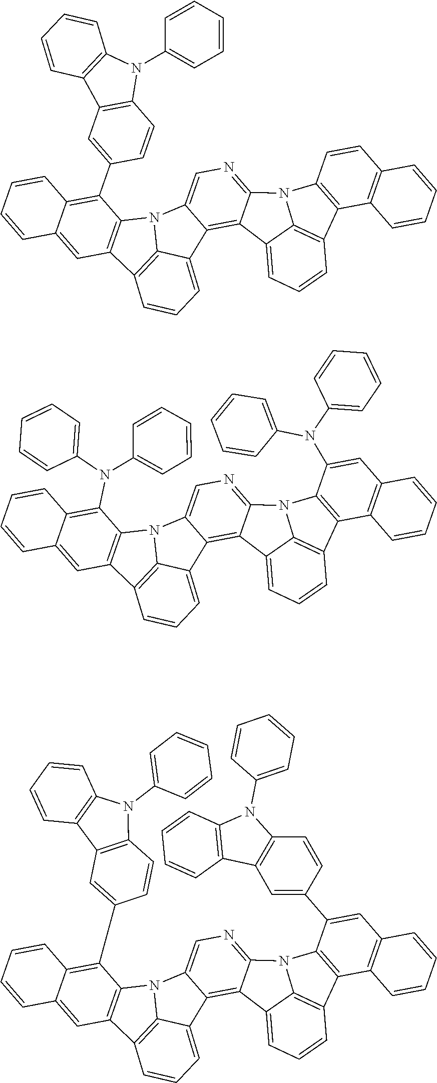 [SMILES: c1ccc(-n2c3ccccc3c3cc(-c4c5ccccc5cc5c6cccc7c8c9c%10cccc%11c%12c%13ccccc%13ccc%12n(c9ncc8n(c45)c67)c%11%10)ccc32)cc1 ; c1ccc(N(c2ccccc2)c2c3ccccc3cc3c4cccc5c6c7c8cccc9c%10c%11ccccc%11cc(N(c%11ccccc%11)c%11ccccc%11)c%10n(c7ncc6n(c23)c45)c89)cc1 ; c1ccc(-n2c3ccccc3c3cc(-c4c5ccccc5cc5c6cccc7c8c9c%10cccc%11c%12c%13ccccc%13cc(-c%13ccc%14c(c%13)c%13ccccc%13n%14-c%13ccccc%13)c%12n(c9ncc8n(c45)c67)c%10%11)ccc32)cc1]

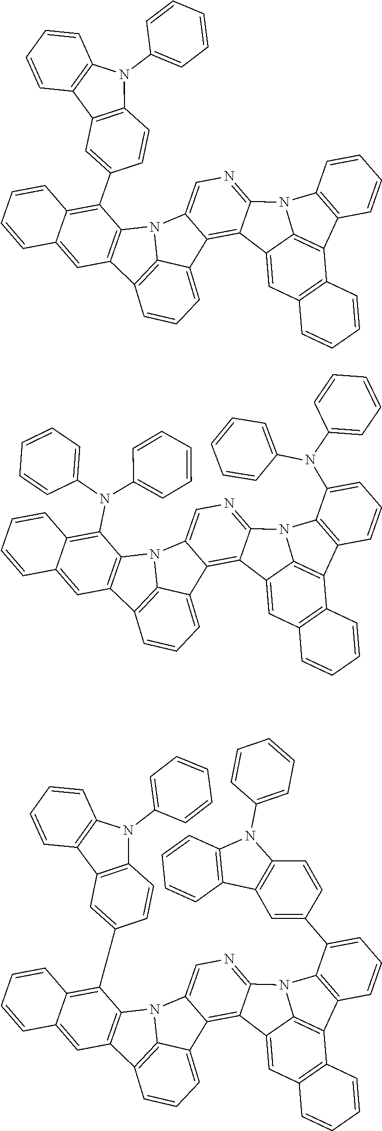

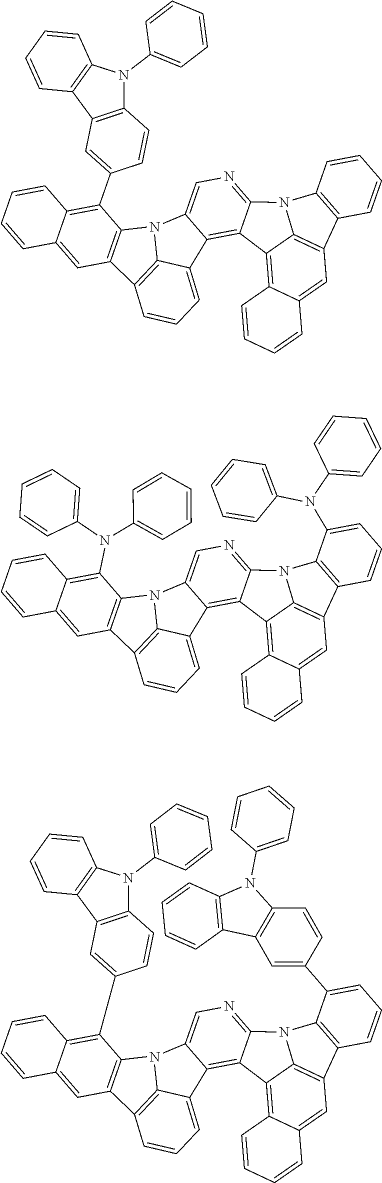 [SMILES: c1ccc(-n2c3ccccc3c3cc(-c4c5ccccc5cc5c6cccc7c8c9c%10c%11ccccc%11cc%11c%12ccccc%12n(c9ncc8n(c45)c67)c%11%10)ccc32)cc1 ; c1ccc(N(c2ccccc2)c2c3ccccc3cc3c4cccc5c6c7c8c9ccccc9cc9c%10cccc(N(c%11ccccc%11)c%11ccccc%11)c%10n(c7ncc6n(c23)c45)c98)cc1 ; c1ccc(-n2c3ccccc3c3cc(-c4c5ccccc5cc5c6cccc7c8c9c%10c%11ccccc%11cc%11c%12cccc(-c%13ccc%14c(c%13)c%13ccccc%13n%14-c%13ccccc%13)c%12n(c9ncc8n(c45)c67)c%11%10)ccc32)cc1]

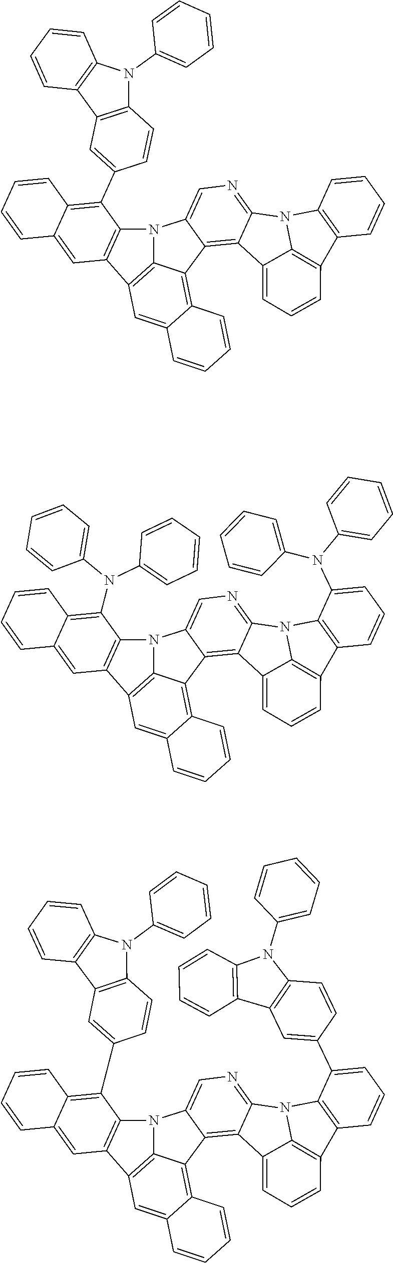 [SMILES: c1ccc(-n2c3ccccc3c3cc(-c4c5ccccc5cc5c6cc7ccccc7c7c8c9c%10cccc%11c%12ccccc%12n(c9ncc8n(c45)c67)c%11%10)ccc32)cc1 ; c1ccc(N(c2ccccc2)c2c3ccccc3cc3c4cc5ccccc5c5c6c7c8cccc9c%10cccc(N(c%11ccccc%11)c%11ccccc%11)c%10n(c7ncc6n(c23)c45)c98)cc1 ; c1ccc(-n2c3ccccc3c3cc(-c4c5ccccc5cc5c6cc7ccccc7c7c8c9c%10cccc%11c%12cccc(-c%13ccc%14c(c%13)c%13ccccc%13n%14-c%13ccccc%13)c%12n(c9ncc8n(c45)c67)c%11%10)ccc32)cc1]

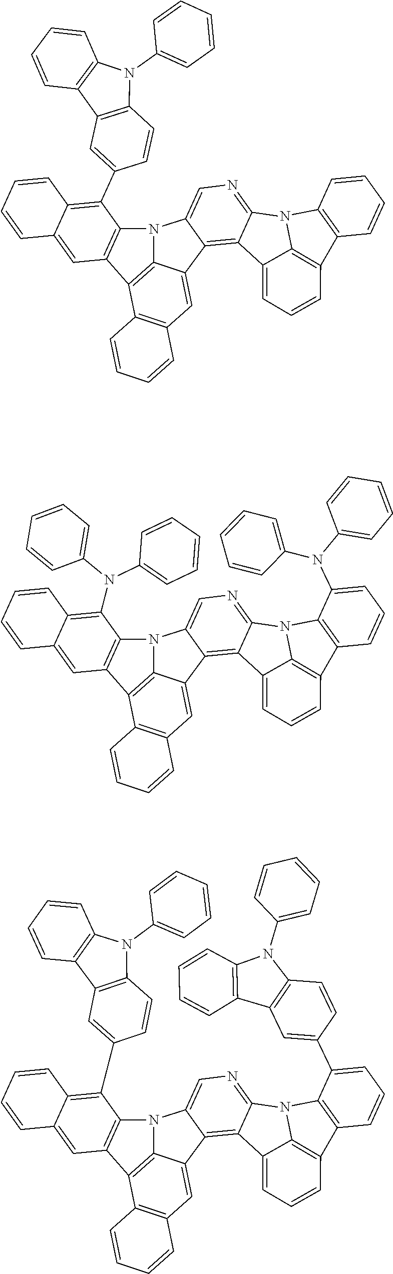 [SMILES: c1ccc(-n2c3ccccc3c3cc(-c4c5ccccc5cc5c6c7ccccc7cc7c8c9c%10cccc%11c%12ccccc%12n(c9ncc8n(c45)c76)c%11%10)ccc32)cc1 ; c1ccc(N(c2ccccc2)c2c3ccccc3cc3c4c5ccccc5cc5c6c7c8cccc9c%10cccc(N(c%11ccccc%11)c%11ccccc%11)c%10n(c7ncc6n(c23)c54)c98)cc1 ; c1ccc(-n2c3ccccc3c3cc(-c4c5ccccc5cc5c6c7ccccc7cc7c8c9c%10cccc%11c%12cccc(-c%13ccc%14c(c%13)c%13ccccc%13n%14-c%13ccccc%13)c%12n(c9ncc8n(c45)c76)c%11%10)ccc32)cc1]

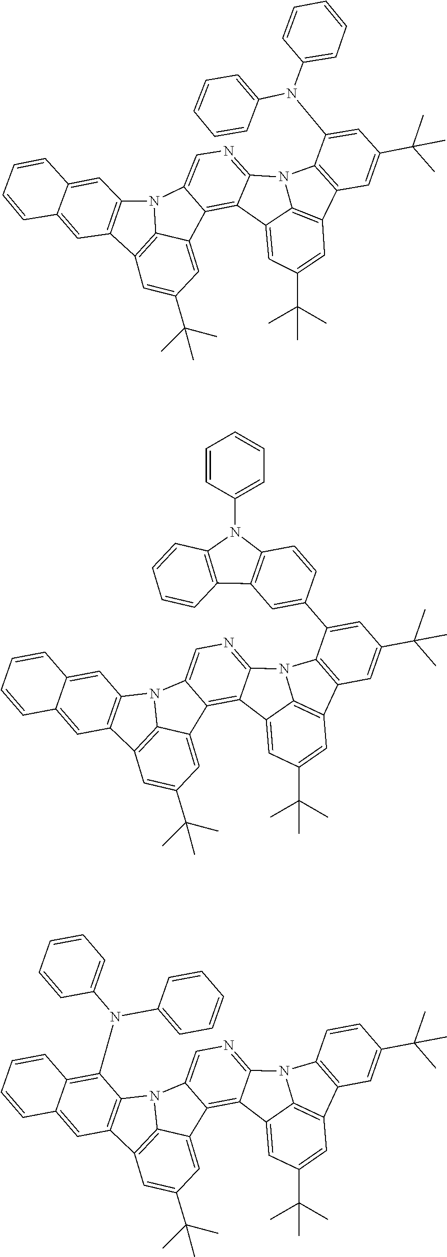 [SMILES: CC(C)(C)c1cc(N(c2ccccc2)c2ccccc2)c2c(c1)c1cc(C(C)(C)C)cc3c4c5c6cc(C(C)(C)C)cc7c8cc9ccccc9cc8n(c5cnc4n2c13)c76 ; CC(C)(C)c1cc(-c2ccc3c(c2)c2ccccc2n3-c2ccccc2)c2c(c1)c1cc(C(C)(C)C)cc3c4c5c6cc(C(C)(C)C)cc7c8cc9ccccc9cc8n(c5cnc4n2c13)c76 ; CC(C)(C)c1ccc2c(c1)c1cc(C(C)(C)C)cc3c4c5c6cc(C(C)(C)C)cc7c8cc9ccccc9c(N(c9ccccc9)c9ccccc9)c8n(c5cnc4n2c13)c76]

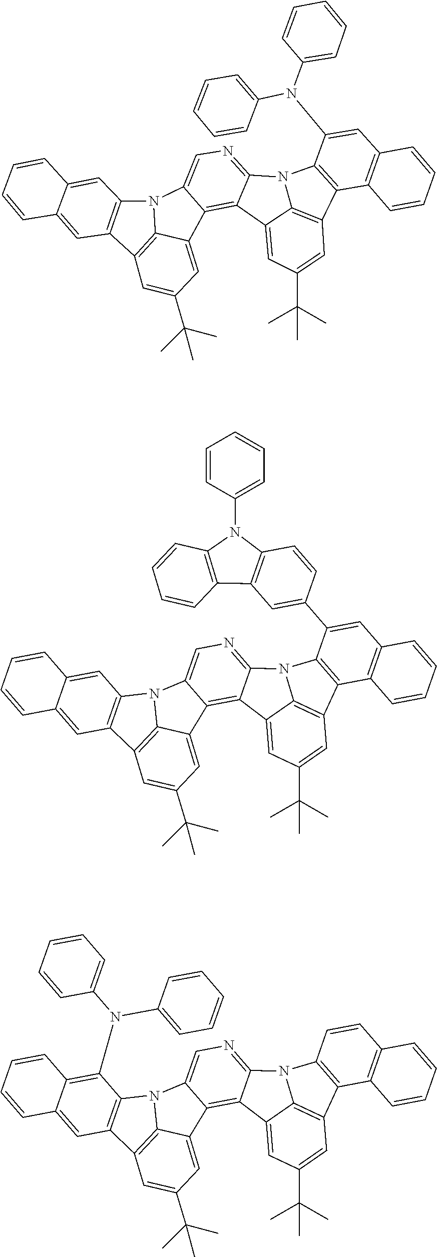 [SMILES: CC(C)(C)c1cc2c3cc4ccccc4cc3n3c4cnc5c(c6cc(C(C)(C)C)cc7c8c9ccccc9cc(N(c9ccccc9)c9ccccc9)c8n5c67)c4c(c1)c23 ; CC(C)(C)c1cc2c3cc4ccccc4cc3n3c4cnc5c(c6cc(C(C)(C)C)cc7c8c9ccccc9cc(-c9ccc%10c(c9)c9ccccc9n%10-c9ccccc9)c8n5c67)c4c(c1)c23 ; CC(C)(C)c1cc2c3c4ccccc4ccc3n3c4ncc5c(c6cc(C(C)(C)C)cc7c8cc9ccccc9c(N(c9ccccc9)c9ccccc9)c8n5c76)c4c(c1)c23]

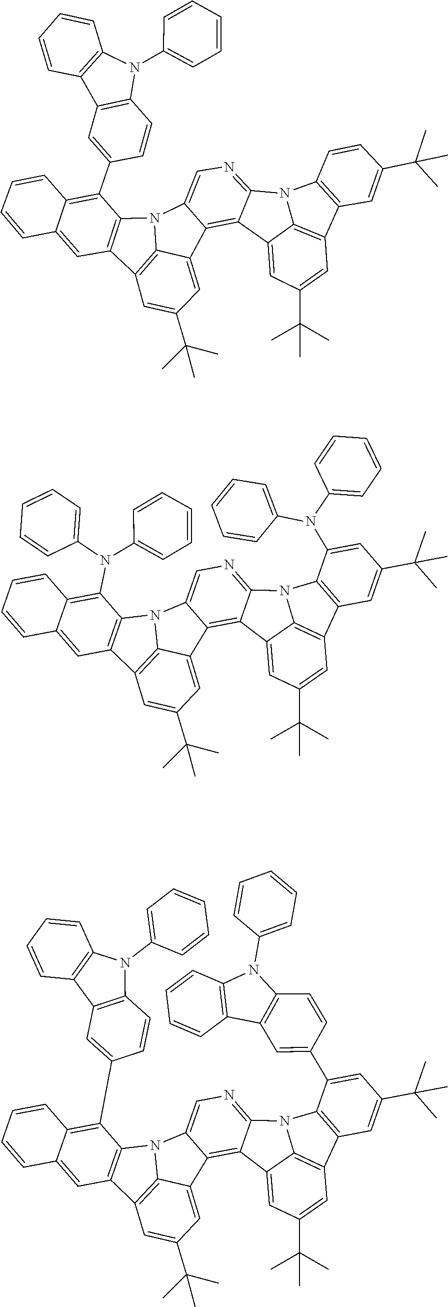 [SMILES: CC(C)(C)c1ccc2c(c1)c1cc(C(C)(C)C)cc3c4c5c6cc(C(C)(C)C)cc7c8cc9ccccc9c(-c9ccc%10c(c9)c9ccccc9n%10-c9ccccc9)c8n(c5cnc4n2c13)c76 ; CC(C)(C)c1cc(N(c2ccccc2)c2ccccc2)c2c(c1)c1cc(C(C)(C)C)cc3c4c5c6cc(C(C)(C)C)cc7c8cc9ccccc9c(N(c9ccccc9)c9ccccc9)c8n(c5cnc4n2c13)c76 ; CC(C)(C)c1cc(-c2ccc3c(c2)c2ccccc2n3-c2ccccc2)c2c(c1)c1cc(C(C)(C)C)cc3c4c5c6cc(C(C)(C)C)cc7c8cc9ccccc9c(-c9ccc%10c(c9)c9ccccc9n%10-c9ccccc9)c8n(c5cnc4n2c13)c76]

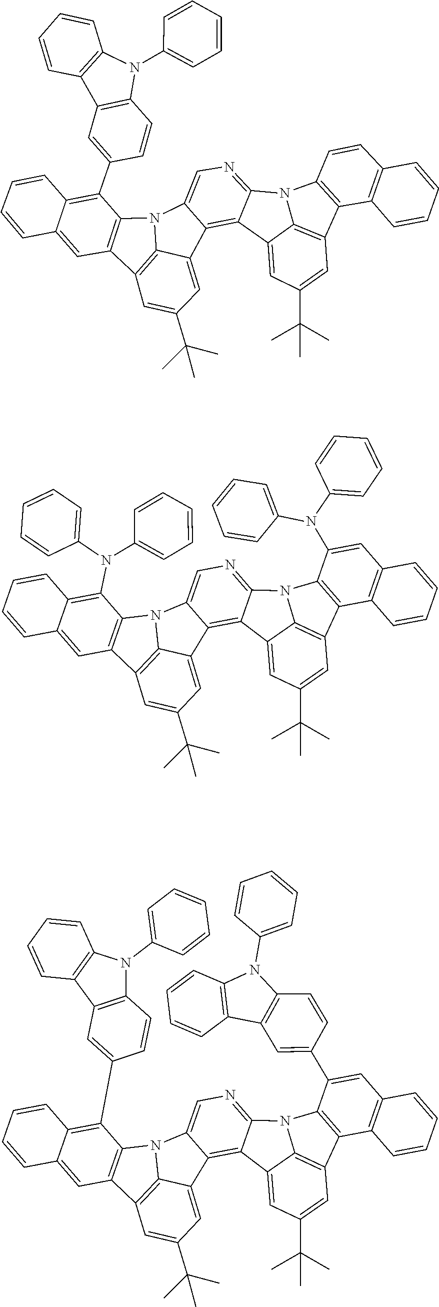 [SMILES: CC(C)(C)c1cc2c3c4ccccc4ccc3n3c4ncc5c(c6cc(C(C)(C)C)cc7c8cc9ccccc9c(-c9ccc%10c(c9)c9ccccc9n%10-c9ccccc9)c8n5c76)c4c(c1)c23 ; CC(C)(C)c1cc2c3cc4ccccc4c(N(c4ccccc4)c4ccccc4)c3n3c4cnc5c(c6cc(C(C)(C)C)cc7c8c9ccccc9cc(N(c9ccccc9)c9ccccc9)c8n5c67)c4c(c1)c23 ; CC(C)(C)c1cc2c3cc4ccccc4c(-c4ccc5c(c4)c4ccccc4n5-c4ccccc4)c3n3c4cnc5c(c6cc(C(C)(C)C)cc7c8c9ccccc9cc(-c9ccc%10c(c9)c9ccccc9n%10-c9ccccc9)c8n5c67)c4c(c1)c23]

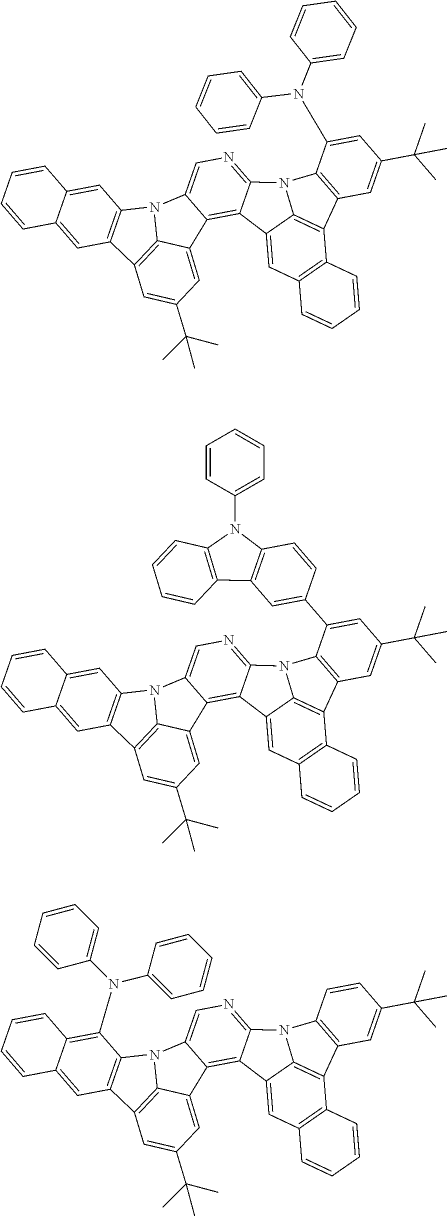 [SMILES: CC(C)(C)c1cc(N(c2ccccc2)c2ccccc2)c2c(c1)c1c3ccccc3cc3c4c5c6cc(C(C)(C)C)cc7c8cc9ccccc9cc8n(c5cnc4n2c31)c76 ; CC(C)(C)c1cc(-c2ccc3c(c2)c2ccccc2n3-c2ccccc2)c2c(c1)c1c3ccccc3cc3c4c5c6cc(C(C)(C)C)cc7c8cc9ccccc9cc8n(c5cnc4n2c31)c76 ; CC(C)(C)c1ccc2c(c1)c1c3ccccc3cc3c4c5c6cc(C(C)(C)C)cc7c8cc9ccccc9c(N(c9ccccc9)c9ccccc9)c8n(c5cnc4n2c31)c76]

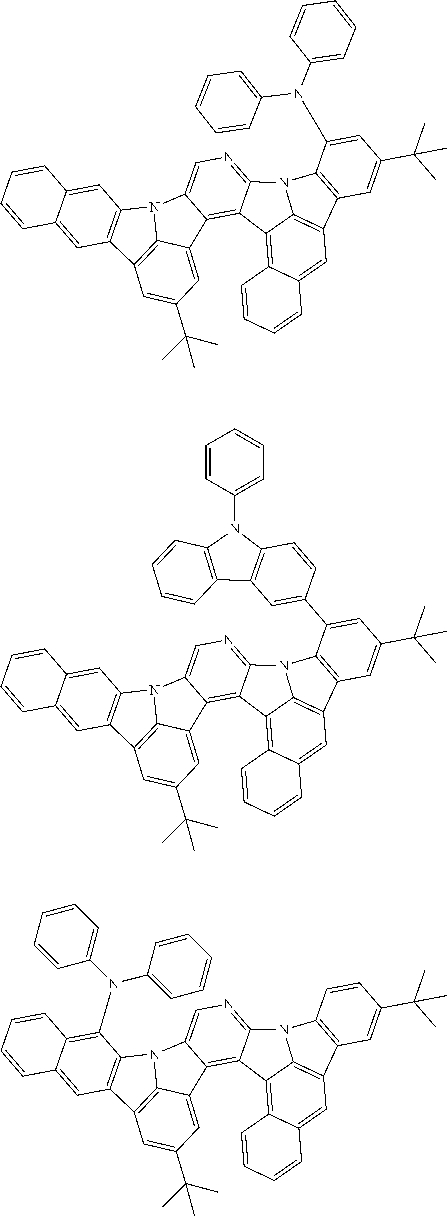 [SMILES: CC(C)(C)c1cc(N(c2ccccc2)c2ccccc2)c2c(c1)c1cc3ccccc3c3c4c5c6cc(C(C)(C)C)cc7c8cc9ccccc9cc8n(c5cnc4n2c13)c76 ; CC(C)(C)c1cc(-c2ccc3c(c2)c2ccccc2n3-c2ccccc2)c2c(c1)c1cc3ccccc3c3c4c5c6cc(C(C)(C)C)cc7c8cc9ccccc9cc8n(c5cnc4n2c13)c76 ; CC(C)(C)c1ccc2c(c1)c1cc3ccccc3c3c4c5c6cc(C(C)(C)C)cc7c8cc9ccccc9c(N(c9ccccc9)c9ccccc9)c8n(c5cnc4n2c13)c76]

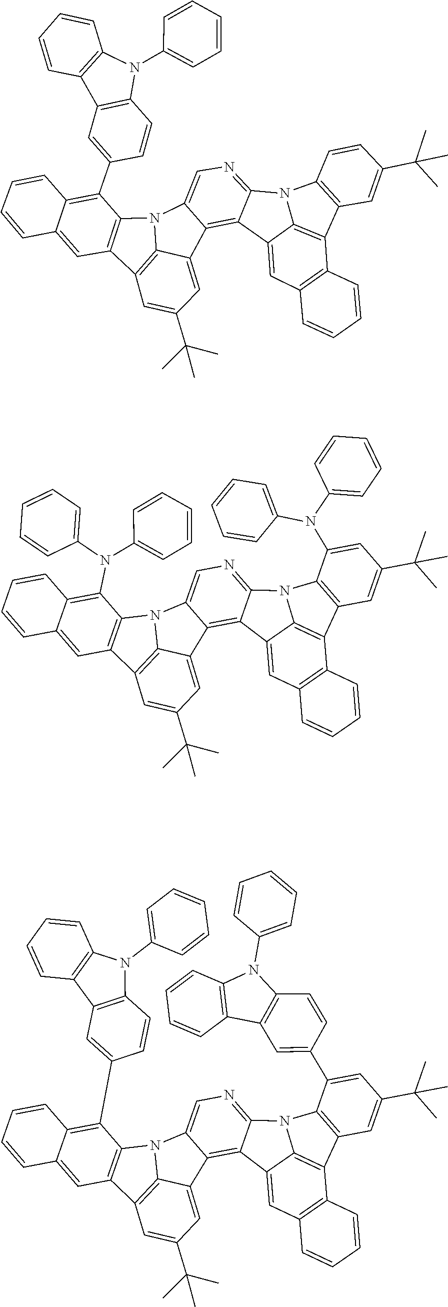 [SMILES: CC(C)(C)c1ccc2c(c1)c1c3ccccc3cc3c4c5c6cc(C(C)(C)C)cc7c8cc9ccccc9c(-c9ccc%10c(c9)c9ccccc9n%10-c9ccccc9)c8n(c5cnc4n2c31)c76 ; CC(C)(C)c1cc(N(c2ccccc2)c2ccccc2)c2c(c1)c1c3ccccc3cc3c4c5c6cc(C(C)(C)C)cc7c8cc9ccccc9c(N(c9ccccc9)c9ccccc9)c8n(c5cnc4n2c31)c76 ; CC(C)(C)c1cc(-c2ccc3c(c2)c2ccccc2n3-c2ccccc2)c2c(c1)c1c3ccccc3cc3c4c5c6cc(C(C)(C)C)cc7c8cc9ccccc9c(-c9ccc%10c(c9)c9ccccc9n%10-c9ccccc9)c8n(c5cnc4n2c31)c76]

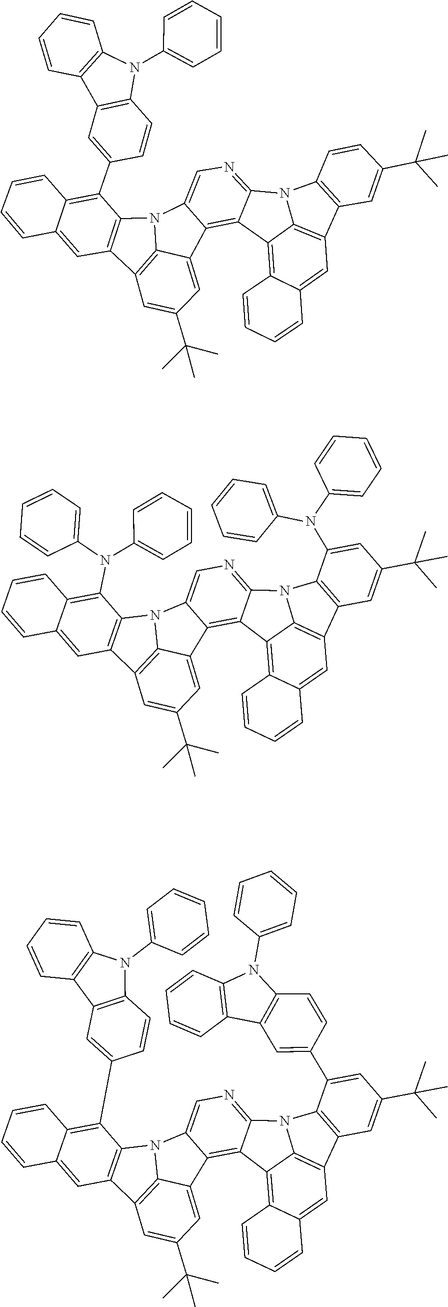 [SMILES: CC(C)(C)c1ccc2c(c1)c1cc3ccccc3c3c4c5c6cc(C(C)(C)C)cc7c8cc9ccccc9c(-c9ccc%10c(c9)c9ccccc9n%10-c9ccccc9)c8n(c5cnc4n2c13)c76 ; CC(C)(C)c1cc(N(c2ccccc2)c2ccccc2)c2c(c1)c1cc3ccccc3c3c4c5c6cc(C(C)(C)C)cc7c8cc9ccccc9c(N(c9ccccc9)c9ccccc9)c8n(c5cnc4n2c13)c76 ; CC(C)(C)c1cc(-c2ccc3c(c2)c2ccccc2n3-c2ccccc2)c2c(c1)c1cc3ccccc3c3c4c5c6cc(C(C)(C)C)cc7c8cc9ccccc9c(-c9ccc%10c(c9)c9ccccc9n%10-c9ccccc9)c8n(c5cnc4n2c13)c76]

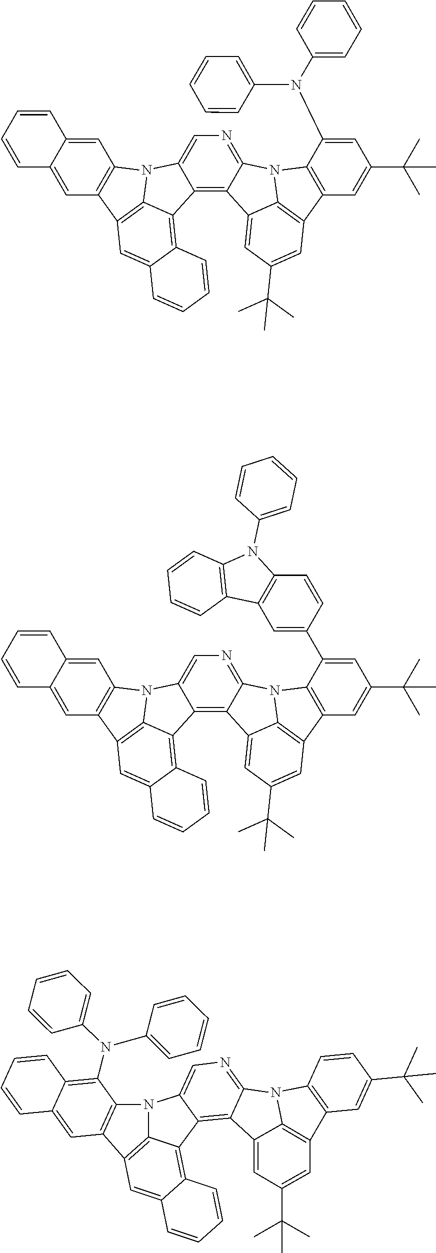 [SMILES: CC(C)(C)c1cc(N(c2ccccc2)c2ccccc2)c2c(c1)c1cc(C(C)(C)C)cc3c4c5c6c7ccccc7cc7c8cc9ccccc9cc8n(c5cnc4n2c13)c76 ; CC(C)(C)c1cc(-c2ccc3c(c2)c2ccccc2n3-c2ccccc2)c2c(c1)c1cc(C(C)(C)C)cc3c4c5c6c7ccccc7cc7c8cc9ccccc9cc8n(c5cnc4n2c13)c76 ; CC(C)(C)c1ccc2c(c1)c1cc(C(C)(C)C)cc3c4c5c6c7ccccc7cc7c8cc9ccccc9c(N(c9ccccc9)c9ccccc9)c8n(c5cnc4n2c13)c76]

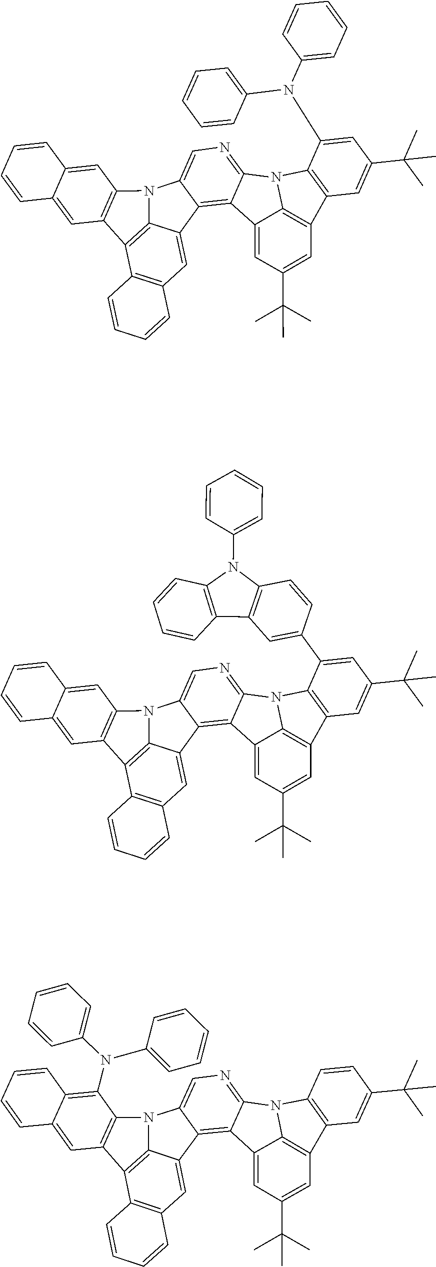 [SMILES: CC(C)(C)c1cc(N(c2ccccc2)c2ccccc2)c2c(c1)c1cc(C(C)(C)C)cc3c4c5c6cc7ccccc7c7c8cc9ccccc9cc8n(c5cnc4n2c13)c67 ; CC(C)(C)c1cc(-c2ccc3c(c2)c2ccccc2n3-c2ccccc2)c2c(c1)c1cc(C(C)(C)C)cc3c4c5c6cc7ccccc7c7c8cc9ccccc9cc8n(c5cnc4n2c13)c67 ; CC(C)(C)c1ccc2c(c1)c1cc(C(C)(C)C)cc3c4c5c6cc7ccccc7c7c8cc9ccccc9c(N(c9ccccc9)c9ccccc9)c8n(c5cnc4n2c13)c67]

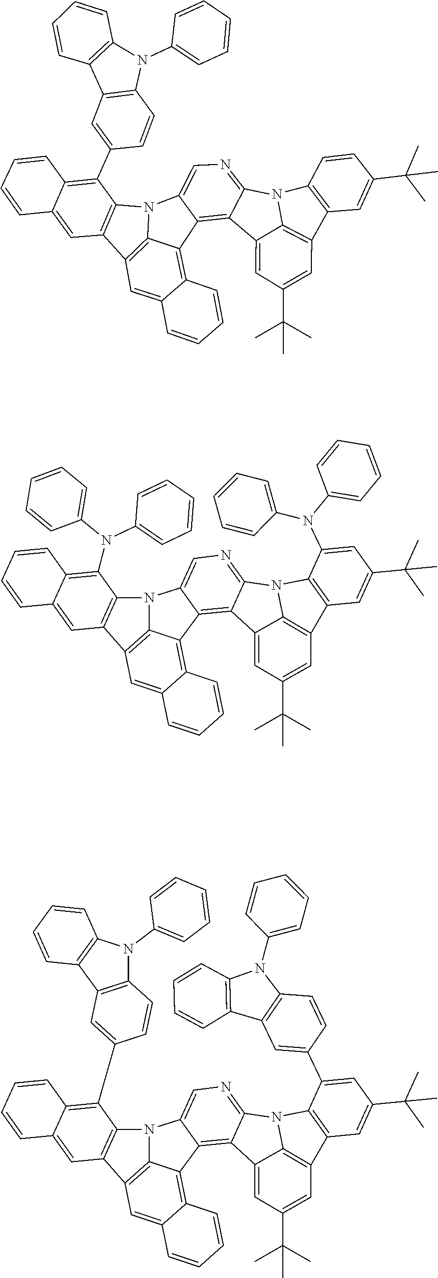 [SMILES: CC(C)(C)c1ccc2c(c1)c1cc(C(C)(C)C)cc3c4c5c6c7ccccc7cc7c8cc9ccccc9c(-c9ccc%10c(c9)c9ccccc9n%10-c9ccccc9)c8n(c5cnc4n2c13)c76 ; CC(C)(C)c1cc(N(c2ccccc2)c2ccccc2)c2c(c1)c1cc(C(C)(C)C)cc3c4c5c6c7ccccc7cc7c8cc9ccccc9c(N(c9ccccc9)c9ccccc9)c8n(c5cnc4n2c13)c76 ; CC(C)(C)c1cc(-c2ccc3c(c2)c2ccccc2n3-c2ccccc2)c2c(c1)c1cc(C(C)(C)C)cc3c4c5c6c7ccccc7cc7c8cc9ccccc9c(-c9ccc%10c(c9)c9ccccc9n%10-c9ccccc9)c8n(c5cnc4n2c13)c76]

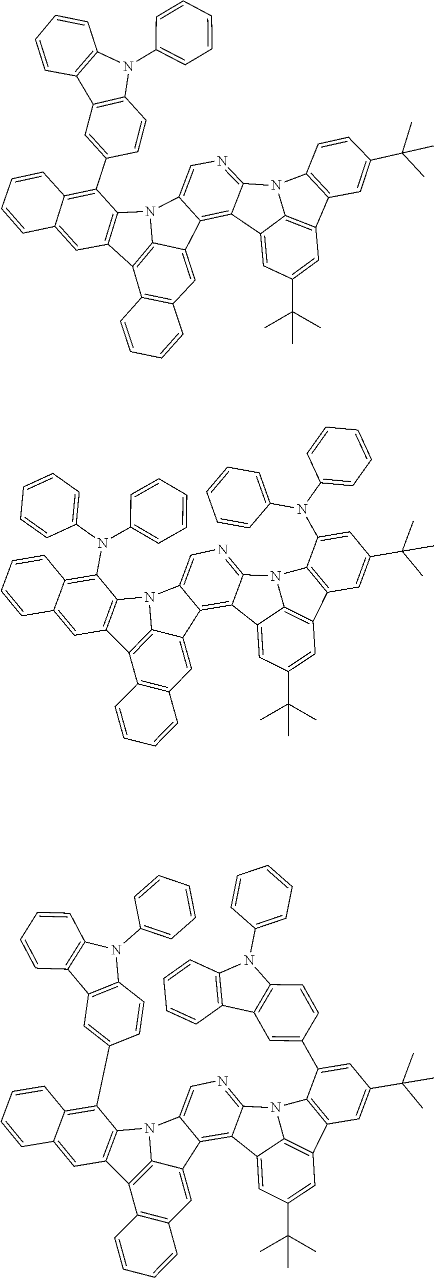 [SMILES: CC(C)(C)c1ccc2c(c1)c1cc(C(C)(C)C)cc3c4c5c6cc7ccccc7c7c8cc9ccccc9c(-c9ccc%10c(c9)c9ccccc9n%10-c9ccccc9)c8n(c5cnc4n2c13)c67 ; CC(C)(C)c1cc(N(c2ccccc2)c2ccccc2)c2c(c1)c1cc(C(C)(C)C)cc3c4c5c6cc7ccccc7c7c8cc9ccccc9c(N(c9ccccc9)c9ccccc9)c8n(c5cnc4n2c13)c67 ; CC(C)(C)c1cc(-c2ccc3c(c2)c2ccccc2n3-c2ccccc2)c2c(c1)c1cc(C(C)(C)C)cc3c4c5c6cc7ccccc7c7c8cc9ccccc9c(-c9ccc%10c(c9)c9ccccc9n%10-c9ccccc9)c8n(c5cnc4n2c13)c67]

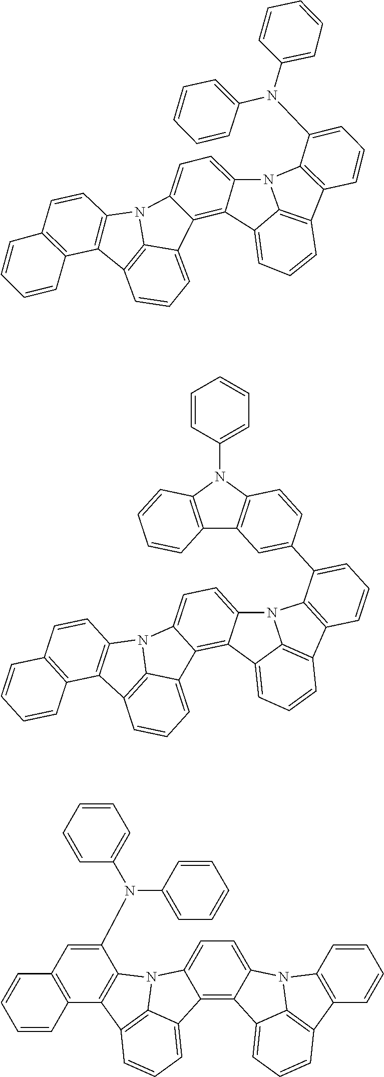 [SMILES: c1ccc(N(c2ccccc2)c2cccc3c4cccc5c6c7c8cccc9c%10c%11ccccc%11ccc%10n(c7ccc6n(c23)c45)c98)cc1 ; c1ccc(-n2c3ccccc3c3cc(-c4cccc5c6cccc7c8c9c%10cccc%11c%12c%13ccccc%13ccc%12n(c9ccc8n(c45)c67)c%11%10)ccc32)cc1 ; c1ccc(N(c2ccccc2)c2cc3ccccc3c3c4cccc5c6c7c8cccc9c%10ccccc%10n(c7ccc6n(c23)c54)c98)cc1]

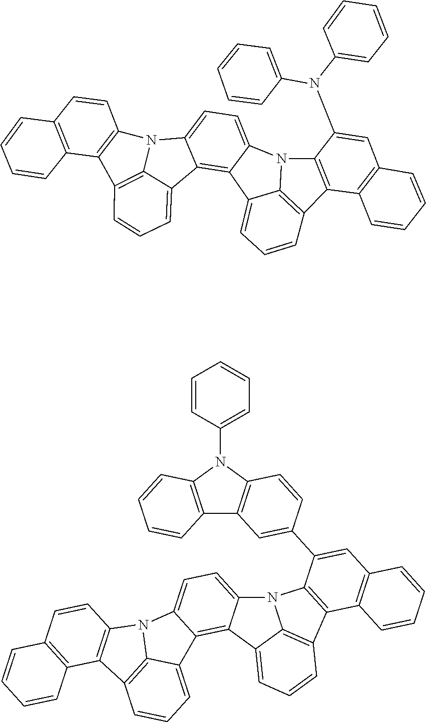 [SMILES: c1ccc(N(c2ccccc2)c2cc3ccccc3c3c4cccc5c6c7c8cccc9c%10c%11ccccc%11ccc%10n(c7ccc6n(c23)c54)c98)cc1 ; c1ccc(-n2c3ccccc3c3cc(-c4cc5ccccc5c5c6cccc7c8c9c%10cccc%11c%12c%13ccccc%13ccc%12n(c9ccc8n(c45)c76)c%11%10)ccc32)cc1]

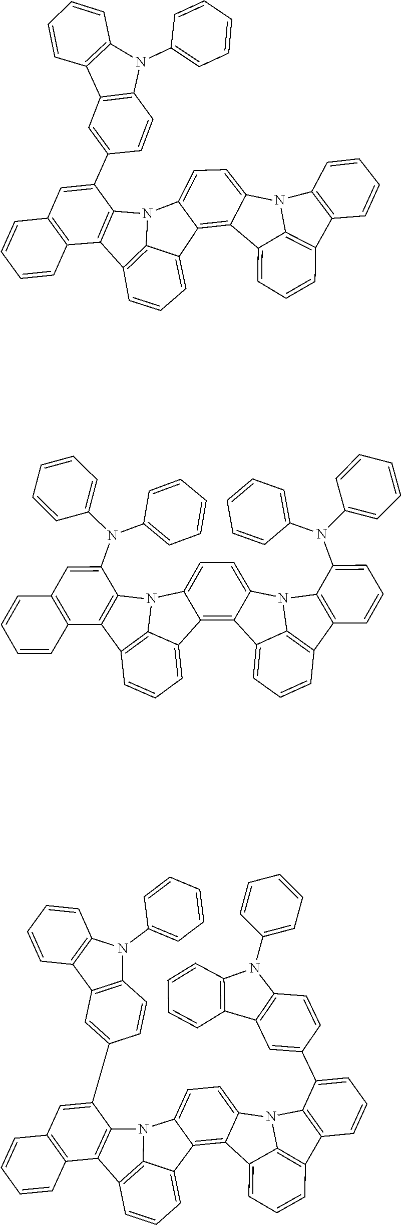 [SMILES: c1ccc(-n2c3ccccc3c3cc(-c4cc5ccccc5c5c6cccc7c8c9c%10cccc%11c%12ccccc%12n(c9ccc8n(c45)c76)c%11%10)ccc32)cc1 ; c1ccc(N(c2ccccc2)c2cccc3c4cccc5c6c7c8cccc9c%10c%11ccccc%11cc(N(c%11ccccc%11)c%11ccccc%11)c%10n(c7ccc6n(c23)c45)c89)cc1 ; c1ccc(-n2c3ccccc3c3cc(-c4cccc5c6cccc7c8c9c%10cccc%11c%12c%13ccccc%13cc(-c%13ccc%14c(c%13)c%13ccccc%13n%14-c%13ccccc%13)c%12n(c9ccc8n(c45)c67)c%10%11)ccc32)cc1]

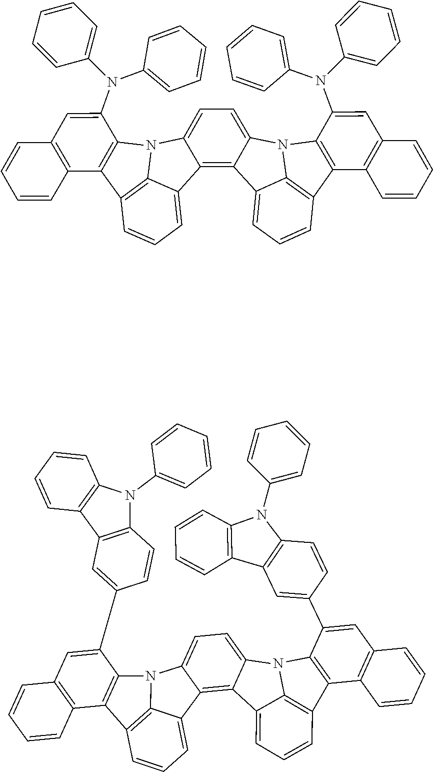 [SMILES: c1ccc(N(c2ccccc2)c2cc3ccccc3c3c4cccc5c6c7c8cccc9c%10c%11ccccc%11cc(N(c%11ccccc%11)c%11ccccc%11)c%10n(c7ccc6n(c23)c54)c89)cc1 ; c1ccc(-n2c3ccccc3c3cc(-c4cc5ccccc5c5c6cccc7c8c9c%10cccc%11c%12c%13ccccc%13cc(-c%13ccc%14c(c%13)c%13ccccc%13n%14-c%13ccccc%13)c%12n(c9ccc8n(c45)c76)c%10%11)ccc32)cc1]

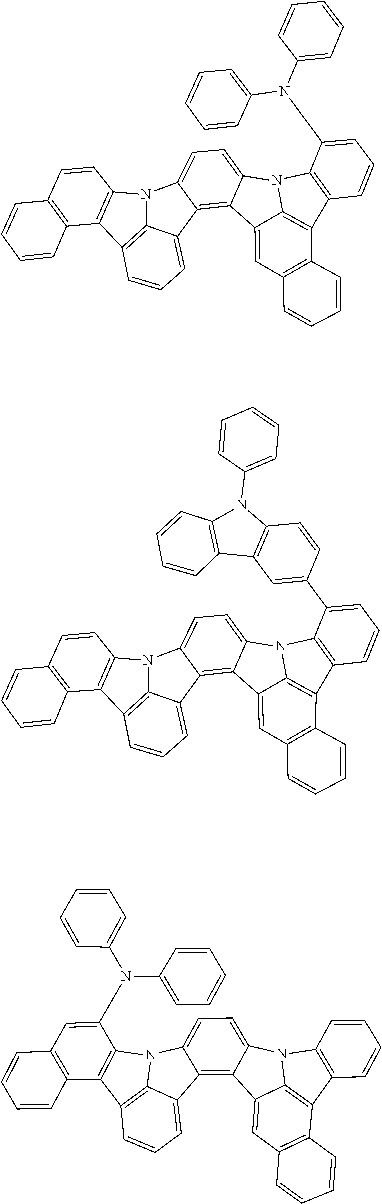 [SMILES: c1ccc(N(c2ccccc2)c2cccc3c4c5ccccc5cc5c6c7c8cccc9c%10c%11ccccc%11ccc%10n(c7ccc6n(c23)c54)c98)cc1 ; c1ccc(-n2c3ccccc3c3cc(-c4cccc5c6c7ccccc7cc7c8c9c%10cccc%11c%12c%13ccccc%13ccc%12n(c9ccc8n(c45)c76)c%11%10)ccc32)cc1 ; c1ccc(N(c2ccccc2)c2cc3ccccc3c3c4cccc5c6c7c8cc9ccccc9c9c%10ccccc%10n(c7ccc6n(c23)c54)c89)cc1]

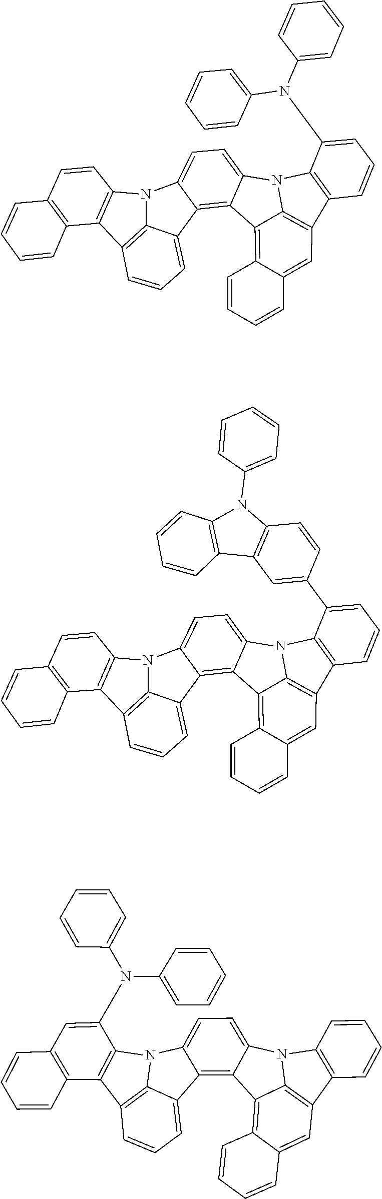 [SMILES: c1ccc(N(c2ccccc2)c2cccc3c4cc5ccccc5c5c6c7c8cccc9c%10c%11ccccc%11ccc%10n(c7ccc6n(c23)c45)c98)cc1 ; c1ccc(-n2c3ccccc3c3cc(-c4cccc5c6cc7ccccc7c7c8c9c%10cccc%11c%12c%13ccccc%13ccc%12n(c9ccc8n(c45)c67)c%11%10)ccc32)cc1 ; c1ccc(N(c2ccccc2)c2cc3ccccc3c3c4cccc5c6c7c8c9ccccc9cc9c%10ccccc%10n(c7ccc6n(c23)c54)c98)cc1]

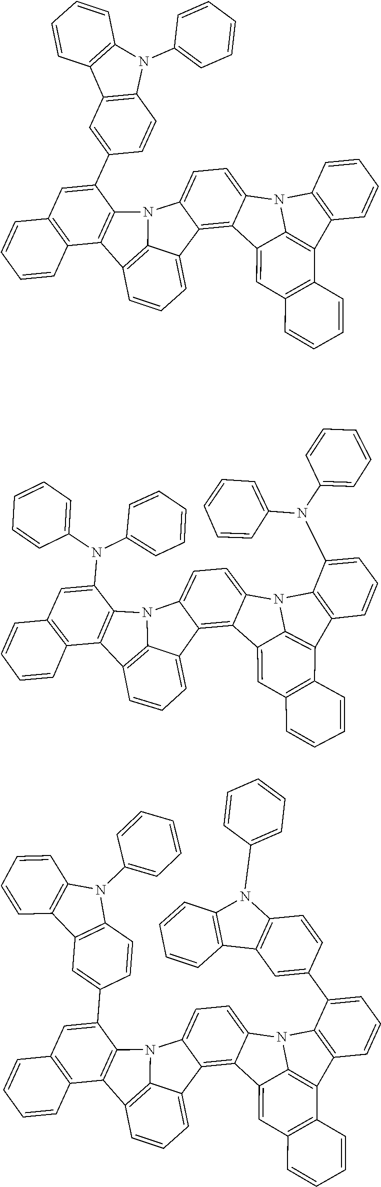 [SMILES: c1ccc(-n2c3ccccc3c3cc(-c4cc5ccccc5c5c6cccc7c8c9c%10cc%11ccccc%11c%11c%12ccccc%12n(c9ccc8n(c45)c76)c%10%11)ccc32)cc1 ; c1ccc(N(c2ccccc2)c2cc3ccccc3c3c4cccc5c6c7c8cc9ccccc9c9c%10cccc(N(c%11ccccc%11)c%11ccccc%11)c%10n(c7ccc6n(c23)c54)c89)cc1 ; c1ccc(-n2c3ccccc3c3cc(-c4cc5ccccc5c5c6cccc7c8c9c%10cc%11ccccc%11c%11c%12cccc(-c%13ccc%14c(c%13)c%13ccccc%13n%14-c%13ccccc%13)c%12n(c9ccc8n(c45)c76)c%10%11)ccc32)cc1]

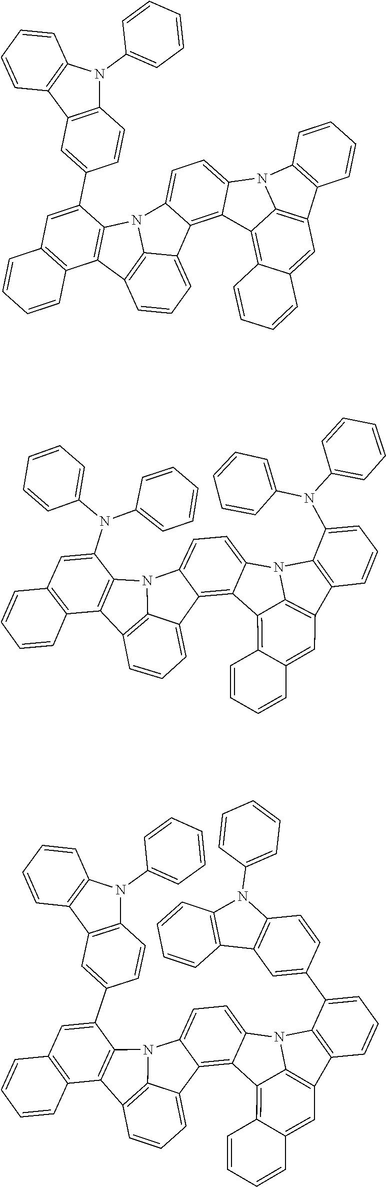 [SMILES: c1ccc(-n2c3ccccc3c3cc(-c4cc5ccccc5c5c6cccc7c8c9c%10c%11ccccc%11cc%11c%12ccccc%12n(c9ccc8n(c45)c76)c%11%10)ccc32)cc1 ; c1ccc(N(c2ccccc2)c2cc3ccccc3c3c4cccc5c6c7c8c9ccccc9cc9c%10cccc(N(c%11ccccc%11)c%11ccccc%11)c%10n(c7ccc6n(c23)c54)c98)cc1 ; c1ccc(-n2c3ccccc3c3cc(-c4cc5ccccc5c5c6cccc7c8c9c%10c%11ccccc%11cc%11c%12cccc(-c%13ccc%14c(c%13)c%13ccccc%13n%14-c%13ccccc%13)c%12n(c9ccc8n(c45)c76)c%11%10)ccc32)cc1]

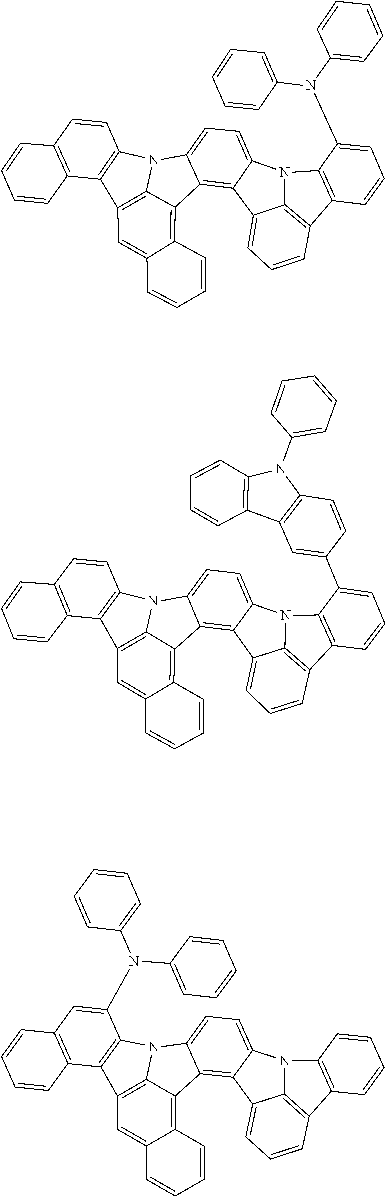 [SMILES: c1ccc(N(c2ccccc2)c2cccc3c4cccc5c6c7c8c9ccccc9cc9c%10c%11ccccc%11ccc%10n(c7ccc6n(c23)c45)c98)cc1 ; c1ccc(-n2c3ccccc3c3cc(-c4cccc5c6cccc7c8c9c%10c%11ccccc%11cc%11c%12c%13ccccc%13ccc%12n(c9ccc8n(c45)c67)c%11%10)ccc32)cc1 ; c1ccc(N(c2ccccc2)c2cc3ccccc3c3c4cc5ccccc5c5c6c7c8cccc9c%10ccccc%10n(c7ccc6n(c23)c45)c98)cc1]

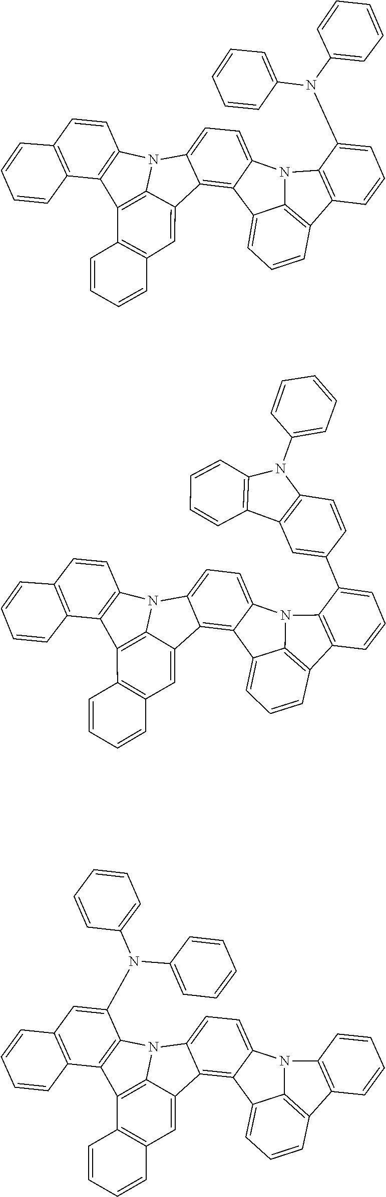 [SMILES: c1ccc(N(c2ccccc2)c2cccc3c4cccc5c6c7c8cc9ccccc9c9c%10c%11ccccc%11ccc%10n(c7ccc6n(c23)c45)c89)cc1 ; c1ccc(-n2c3ccccc3c3cc(-c4cccc5c6cccc7c8c9c%10cc%11ccccc%11c%11c%12c%13ccccc%13ccc%12n(c9ccc8n(c45)c67)c%10%11)ccc32)cc1 ; c1ccc(N(c2ccccc2)c2cc3ccccc3c3c4c5ccccc5cc5c6c7c8cccc9c%10ccccc%10n(c7ccc6n(c23)c54)c98)cc1]

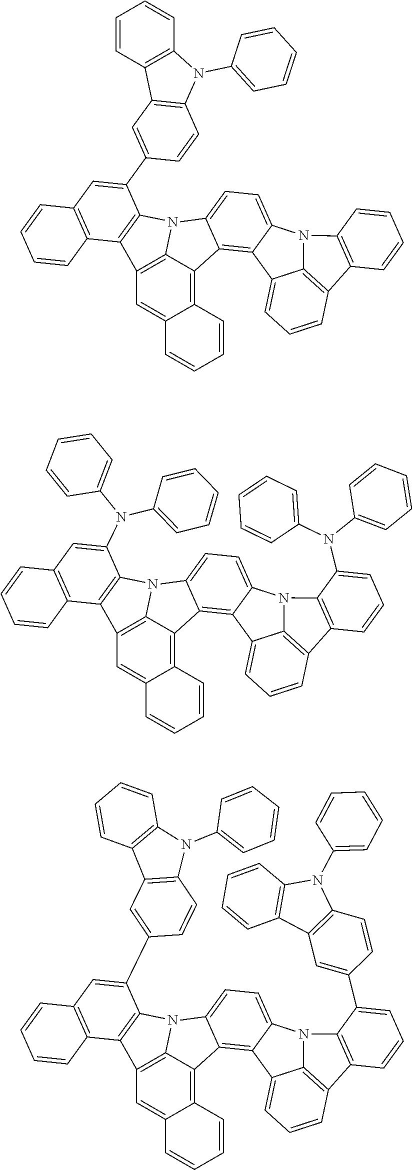 [SMILES: c1ccc(-n2c3ccccc3c3cc(-c4cc5ccccc5c5c6cc7ccccc7c7c8c9c%10cccc%11c%12ccccc%12n(c9ccc8n(c45)c67)c%11%10)ccc32)cc1 ; c1ccc(N(c2ccccc2)c2cccc3c4cccc5c6c7c8c9ccccc9cc9c%10c%11ccccc%11cc(N(c%11ccccc%11)c%11ccccc%11)c%10n(c7ccc6n(c23)c45)c98)cc1 ; c1ccc(-n2c3ccccc3c3cc(-c4cccc5c6cccc7c8c9c%10c%11ccccc%11cc%11c%12c%13ccccc%13cc(-c%13ccc%14c(c%13)c%13ccccc%13n%14-c%13ccccc%13)c%12n(c9ccc8n(c45)c67)c%11%10)ccc32)cc1]

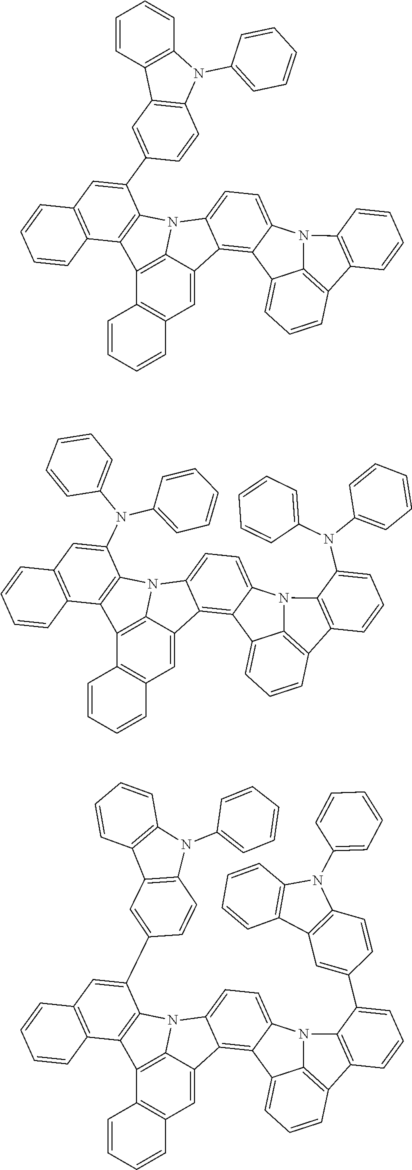 [SMILES: c1ccc(-n2c3ccccc3c3cc(-c4cc5ccccc5c5c6c7ccccc7cc7c8c9c%10cccc%11c%12ccccc%12n(c9ccc8n(c45)c76)c%11%10)ccc32)cc1 ; c1ccc(N(c2ccccc2)c2cccc3c4cccc5c6c7c8cc9ccccc9c9c%10c%11ccccc%11cc(N(c%11ccccc%11)c%11ccccc%11)c%10n(c7ccc6n(c23)c45)c89)cc1 ; c1ccc(-n2c3ccccc3c3cc(-c4cccc5c6cccc7c8c9c%10cc%11ccccc%11c%11c%12c%13ccccc%13cc(-c%13ccc%14c(c%13)c%13ccccc%13n%14-c%13ccccc%13)c%12n(c9ccc8n(c45)c67)c%10%11)ccc32)cc1]

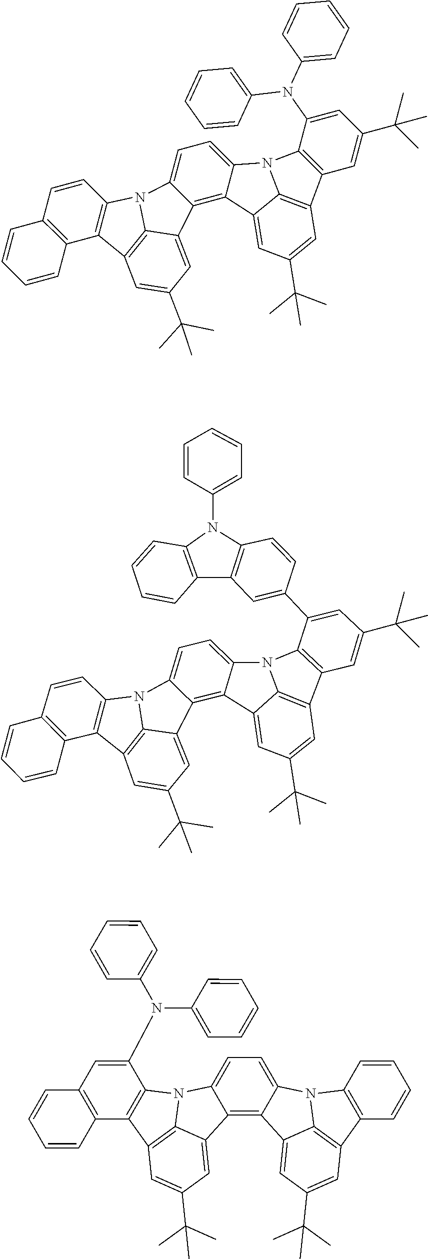 [SMILES: CC(C)(C)c1cc(N(c2ccccc2)c2ccccc2)c2c(c1)c1cc(C(C)(C)C)cc3c4c5c6cc(C(C)(C)C)cc7c8c9ccccc9ccc8n(c5ccc4n2c13)c76 ; CC(C)(C)c1cc(-c2ccc3c(c2)c2ccccc2n3-c2ccccc2)c2c(c1)c1cc(C(C)(C)C)cc3c4c5c6cc(C(C)(C)C)cc7c8c9ccccc9ccc8n(c5ccc4n2c13)c76 ; CC(C)(C)c1cc2c3ccccc3n3c4ccc5c(c6cc(C(C)(C)C)cc7c8c9ccccc9cc(N(c9ccccc9)c9ccccc9)c8n5c67)c4c(c1)c23]

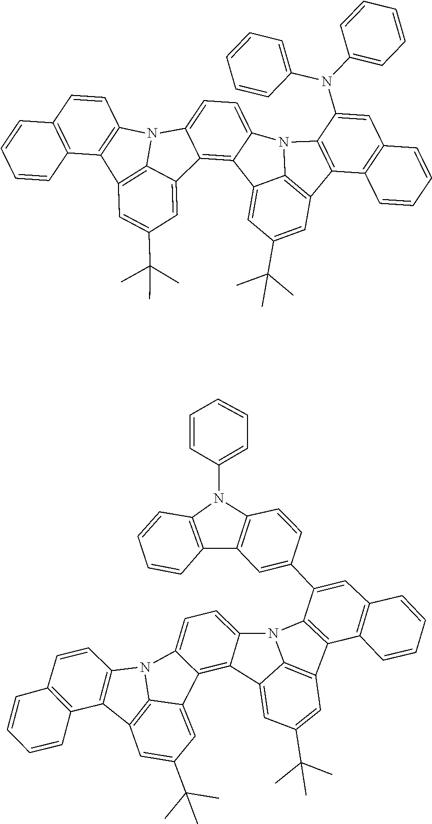 [SMILES: CC(C)(C)c1cc2c3c4ccccc4ccc3n3c4ccc5c(c6cc(C(C)(C)C)cc7c8c9ccccc9cc(N(c9ccccc9)c9ccccc9)c8n5c67)c4c(c1)c23 ; CC(C)(C)c1cc2c3c4ccccc4ccc3n3c4ccc5c(c6cc(C(C)(C)C)cc7c8c9ccccc9cc(-c9ccc%10c(c9)c9ccccc9n%10-c9ccccc9)c8n5c67)c4c(c1)c23]

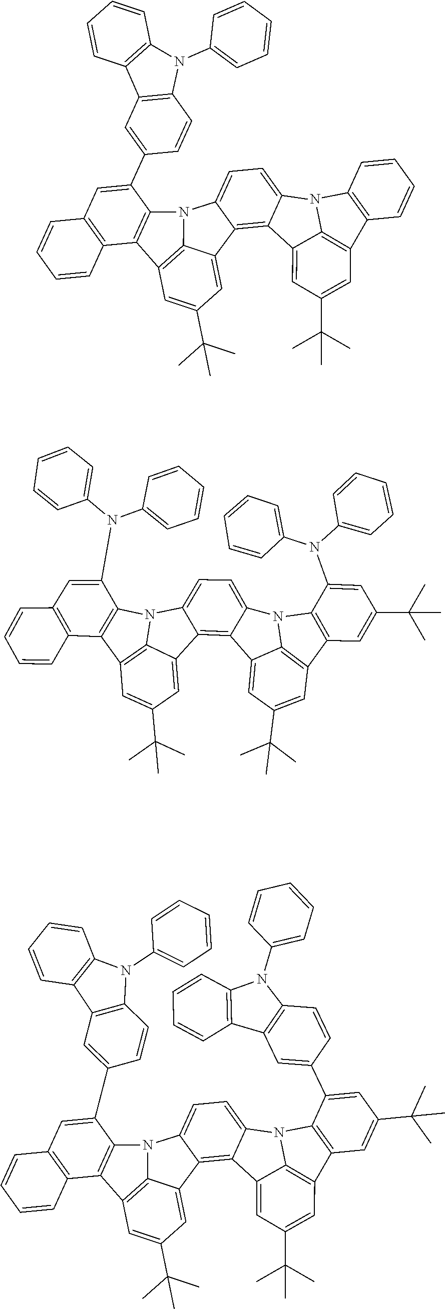 [SMILES: CC(C)(C)c1cc2c3ccccc3n3c4ccc5c(c6cc(C(C)(C)C)cc7c8c9ccccc9cc(-c9ccc%10c(c9)c9ccccc9n%10-c9ccccc9)c8n5c67)c4c(c1)c23 ; CC(C)(C)c1cc(N(c2ccccc2)c2ccccc2)c2c(c1)c1cc(C(C)(C)C)cc3c4c5c6cc(C(C)(C)C)cc7c8c9ccccc9cc(N(c9ccccc9)c9ccccc9)c8n(c5ccc4n2c13)c67 ; CC(C)(C)c1cc(-c2ccc3c(c2)c2ccccc2n3-c2ccccc2)c2c(c1)c1cc(C(C)(C)C)cc3c4c5c6cc(C(C)(C)C)cc7c8c9ccccc9cc(-c9ccc%10c(c9)c9ccccc9n%10-c9ccccc9)c8n(c5ccc4n2c13)c67]

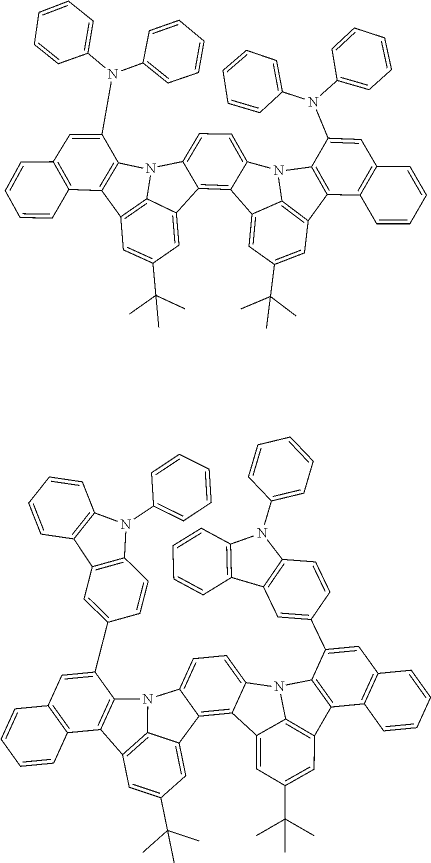 [SMILES: CC(C)(C)c1cc2c3c4c5cc(C(C)(C)C)cc6c7c8ccccc8cc(N(c8ccccc8)c8ccccc8)c7n(c4ccc3n3c4c(N(c7ccccc7)c7ccccc7)cc7ccccc7c4c(c1)c23)c56 ; CC(C)(C)c1cc2c3c4c5cc(C(C)(C)C)cc6c7c8ccccc8cc(-c8ccc9c(c8)c8ccccc8n9-c8ccccc8)c7n(c4ccc3n3c4c(-c7ccc8c(c7)c7ccccc7n8-c7ccccc7)cc7ccccc7c4c(c1)c23)c56]

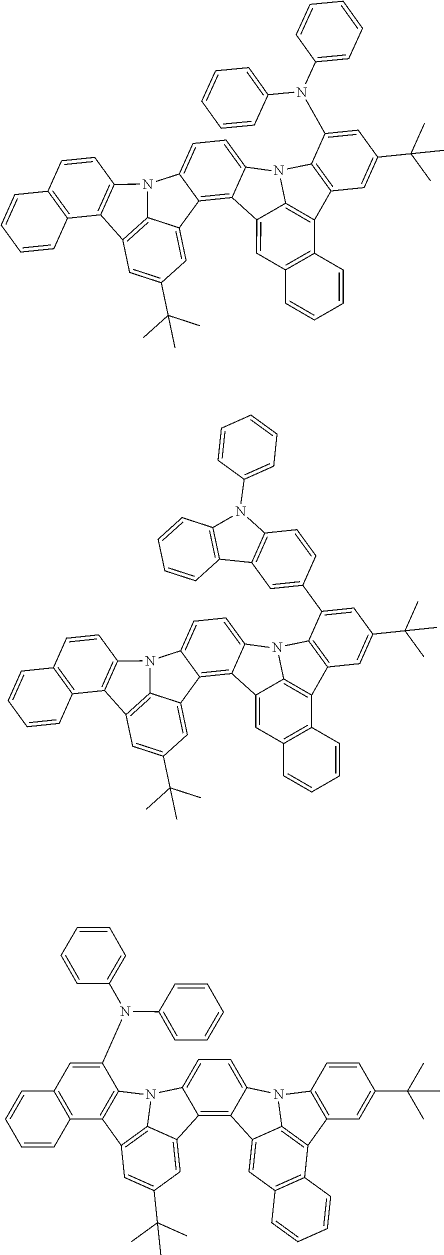 [SMILES: CC(C)(C)c1cc(N(c2ccccc2)c2ccccc2)c2c(c1)c1c3ccccc3cc3c4c5c6cc(C(C)(C)C)cc7c8c9ccccc9ccc8n(c5ccc4n2c31)c76 ; CC(C)(C)c1cc(-c2ccc3c(c2)c2ccccc2n3-c2ccccc2)c2c(c1)c1c3ccccc3cc3c4c5c6cc(C(C)(C)C)cc7c8c9ccccc9ccc8n(c5ccc4n2c31)c76 ; CC(C)(C)c1ccc2c(c1)c1c3ccccc3cc3c4c5c6cc(C(C)(C)C)cc7c8c9ccccc9cc(N(c9ccccc9)c9ccccc9)c8n(c5ccc4n2c31)c67]

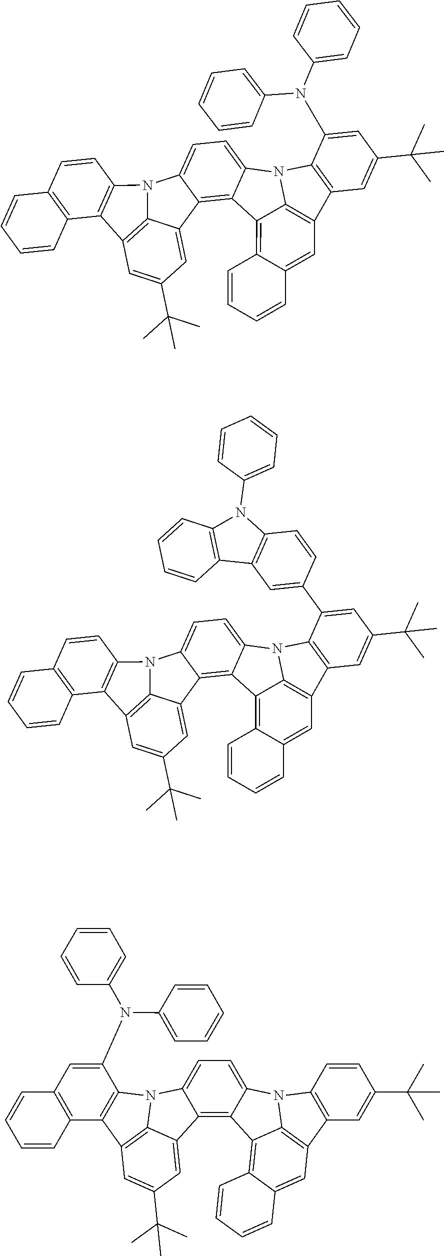 [SMILES: CC(C)(C)c1cc(N(c2ccccc2)c2ccccc2)c2c(c1)c1cc3ccccc3c3c4c5c6cc(C(C)(C)C)cc7c8c9ccccc9ccc8n(c5ccc4n2c13)c76 ; CC(C)(C)c1cc(-c2ccc3c(c2)c2ccccc2n3-c2ccccc2)c2c(c1)c1cc3ccccc3c3c4c5c6cc(C(C)(C)C)cc7c8c9ccccc9ccc8n(c5ccc4n2c13)c76 ; CC(C)(C)c1ccc2c(c1)c1cc3ccccc3c3c4c5c6cc(C(C)(C)C)cc7c8c9ccccc9cc(N(c9ccccc9)c9ccccc9)c8n(c5ccc4n2c13)c67]

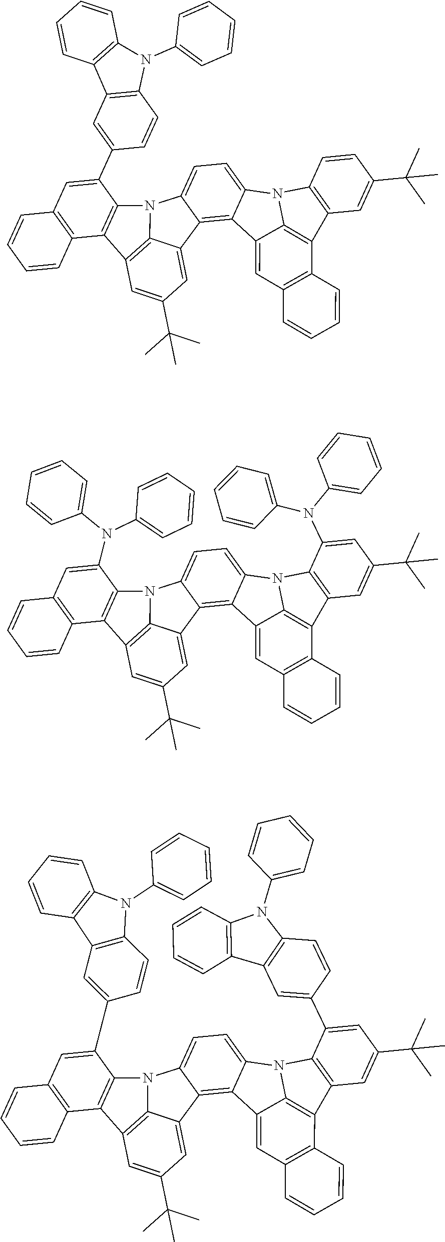 [SMILES: CC(C)(C)c1ccc2c(c1)c1c3ccccc3cc3c4c5c6cc(C(C)(C)C)cc7c8c9ccccc9cc(-c9ccc%10c(c9)c9ccccc9n%10-c9ccccc9)c8n(c5ccc4n2c31)c67 ; CC(C)(C)c1cc(N(c2ccccc2)c2ccccc2)c2c(c1)c1c3ccccc3cc3c4c5c6cc(C(C)(C)C)cc7c8c9ccccc9cc(N(c9ccccc9)c9ccccc9)c8n(c5ccc4n2c31)c67 ; CC(C)(C)c1cc(-c2ccc3c(c2)c2ccccc2n3-c2ccccc2)c2c(c1)c1c3ccccc3cc3c4c5c6cc(C(C)(C)C)cc7c8c9ccccc9cc(-c9ccc%10c(c9)c9ccccc9n%10-c9ccccc9)c8n(c5ccc4n2c31)c67]

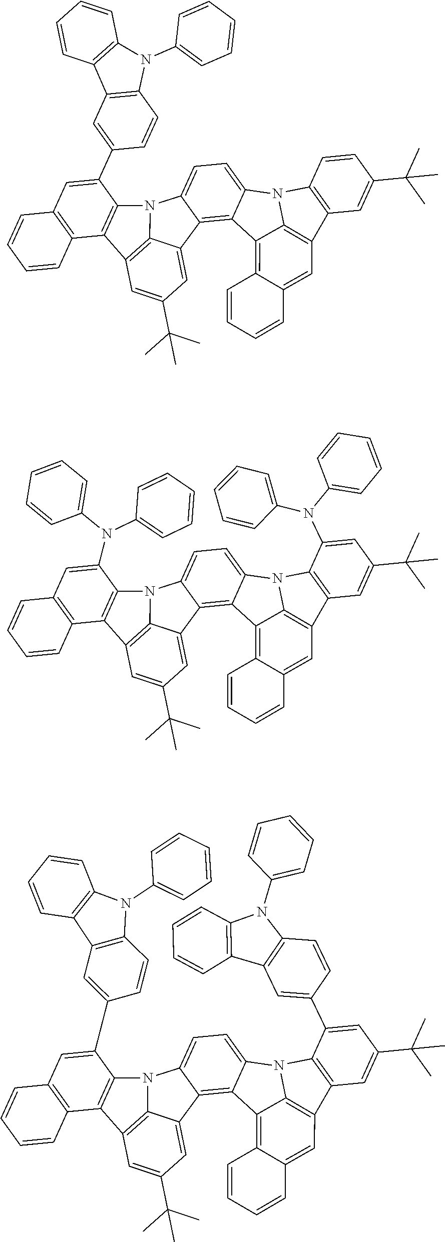 [SMILES: CC(C)(C)c1ccc2c(c1)c1cc3ccccc3c3c4c5c6cc(C(C)(C)C)cc7c8c9ccccc9cc(-c9ccc%10c(c9)c9ccccc9n%10-c9ccccc9)c8n(c5ccc4n2c13)c67 ; CC(C)(C)c1cc(N(c2ccccc2)c2ccccc2)c2c(c1)c1cc3ccccc3c3c4c5c6cc(C(C)(C)C)cc7c8c9ccccc9cc(N(c9ccccc9)c9ccccc9)c8n(c5ccc4n2c13)c67 ; CC(C)(C)c1cc(-c2ccc3c(c2)c2ccccc2n3-c2ccccc2)c2c(c1)c1cc3ccccc3c3c4c5c6cc(C(C)(C)C)cc7c8c9ccccc9cc(-c9ccc%10c(c9)c9ccccc9n%10-c9ccccc9)c8n(c5ccc4n2c13)c67]

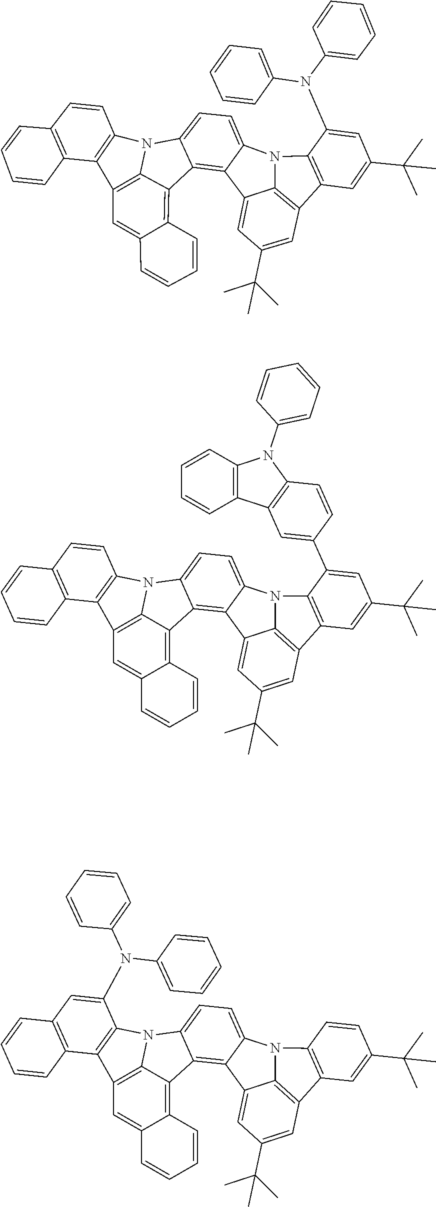 [SMILES: CC(C)(C)c1cc(N(c2ccccc2)c2ccccc2)c2c(c1)c1cc(C(C)(C)C)cc3c4c5c6c7ccccc7cc7c8c9ccccc9ccc8n(c5ccc4n2c13)c76 ; CC(C)(C)c1cc(-c2ccc3c(c2)c2ccccc2n3-c2ccccc2)c2c(c1)c1cc(C(C)(C)C)cc3c4c5c6c7ccccc7cc7c8c9ccccc9ccc8n(c5ccc4n2c13)c76 ; CC(C)(C)c1ccc2c(c1)c1cc(C(C)(C)C)cc3c4c5c6c7ccccc7cc7c8c9ccccc9cc(N(c9ccccc9)c9ccccc9)c8n(c5ccc4n2c13)c76]

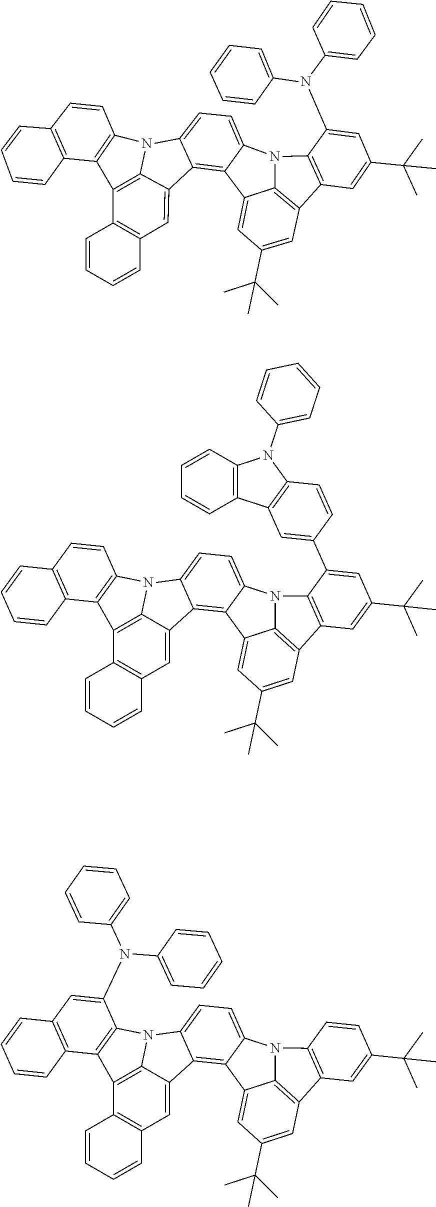 [SMILES: CC(C)(C)c1cc(N(c2ccccc2)c2ccccc2)c2c(c1)c1cc(C(C)(C)C)cc3c4c5c6cc7ccccc7c7c8c9ccccc9ccc8n(c5ccc4n2c13)c67 ; CC(C)(C)c1cc(-c2ccc3c(c2)c2ccccc2n3-c2ccccc2)c2c(c1)c1cc(C(C)(C)C)cc3c4c5c6cc7ccccc7c7c8c9ccccc9ccc8n(c5ccc4n2c13)c67 ; CC(C)(C)c1ccc2c(c1)c1cc(C(C)(C)C)cc3c4c5c6cc7ccccc7c7c8c9ccccc9cc(N(c9ccccc9)c9ccccc9)c8n(c5ccc4n2c13)c67]

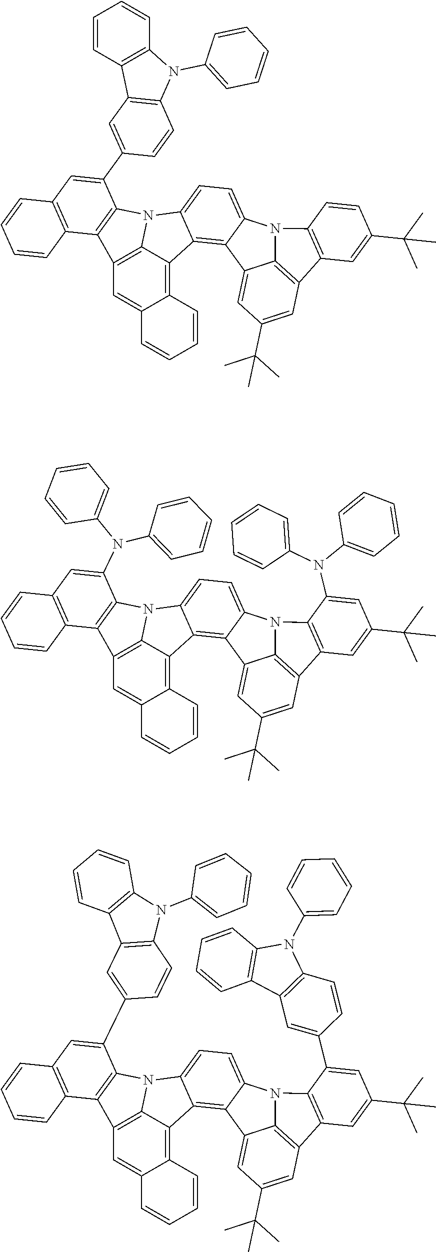 [SMILES: CC(C)(C)c1ccc2c(c1)c1cc(C(C)(C)C)cc3c4c5c6c7ccccc7cc7c8c9ccccc9cc(-c9ccc%10c(c9)c9ccccc9n%10-c9ccccc9)c8n(c5ccc4n2c13)c76 ; CC(C)(C)c1cc(N(c2ccccc2)c2ccccc2)c2c(c1)c1cc(C(C)(C)C)cc3c4c5c6c7ccccc7cc7c8c9ccccc9cc(N(c9ccccc9)c9ccccc9)c8n(c5ccc4n2c13)c76 ; CC(C)(C)c1cc(-c2ccc3c(c2)c2ccccc2n3-c2ccccc2)c2c(c1)c1cc(C(C)(C)C)cc3c4c5c6c7ccccc7cc7c8c9ccccc9cc(-c9ccc%10c(c9)c9ccccc9n%10-c9ccccc9)c8n(c5ccc4n2c13)c76]

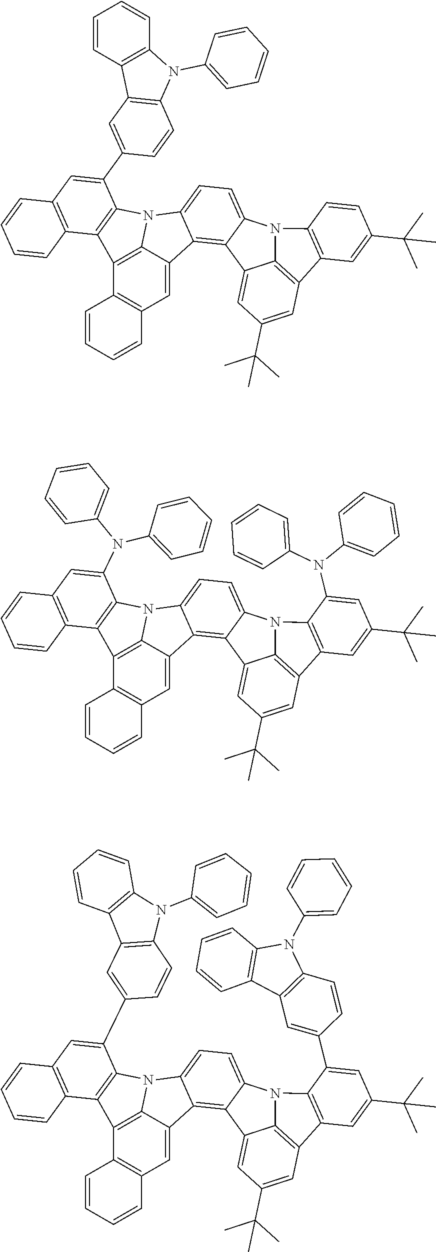 [SMILES: CC(C)(C)c1ccc2c(c1)c1cc(C(C)(C)C)cc3c4c5c6cc7ccccc7c7c8c9ccccc9cc(-c9ccc%10c(c9)c9ccccc9n%10-c9ccccc9)c8n(c5ccc4n2c13)c67 ; CC(C)(C)c1cc(N(c2ccccc2)c2ccccc2)c2c(c1)c1cc(C(C)(C)C)cc3c4c5c6cc7ccccc7c7c8c9ccccc9cc(N(c9ccccc9)c9ccccc9)c8n(c5ccc4n2c13)c67 ; CC(C)(C)c1cc(-c2ccc3c(c2)c2ccccc2n3-c2ccccc2)c2c(c1)c1cc(C(C)(C)C)cc3c4c5c6cc7ccccc7c7c8c9ccccc9cc(-c9ccc%10c(c9)c9ccccc9n%10-c9ccccc9)c8n(c5ccc4n2c13)c67]

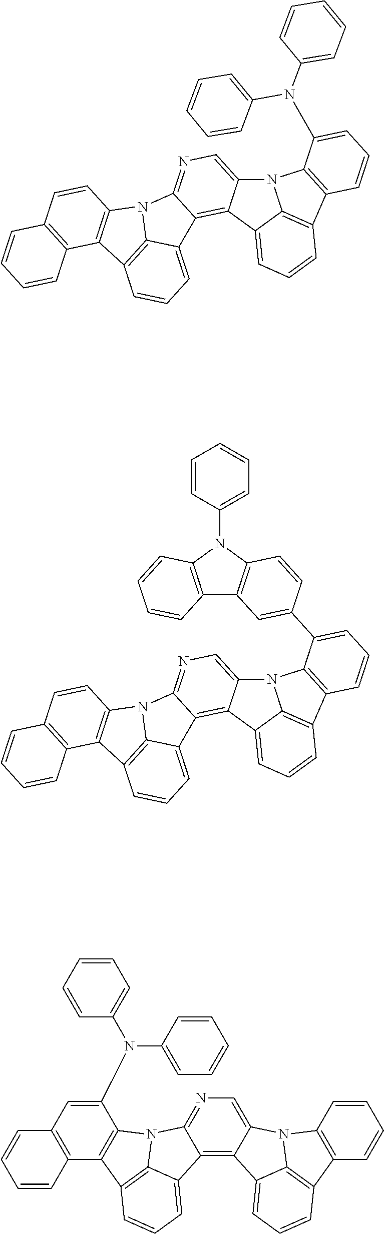 [SMILES: c1ccc(N(c2ccccc2)c2cccc3c4cccc5c6c7c8cccc9c%10c%11ccccc%11ccc%10n(c7ncc6n(c23)c45)c98)cc1 ; c1ccc(-n2c3ccccc3c3cc(-c4cccc5c6cccc7c8c9c%10cccc%11c%12c%13ccccc%13ccc%12n(c9ncc8n(c45)c67)c%11%10)ccc32)cc1 ; c1ccc(N(c2ccccc2)c2cc3ccccc3c3c4cccc5c6c7c8cccc9c%10ccccc%10n(c7cnc6n(c23)c54)c98)cc1]

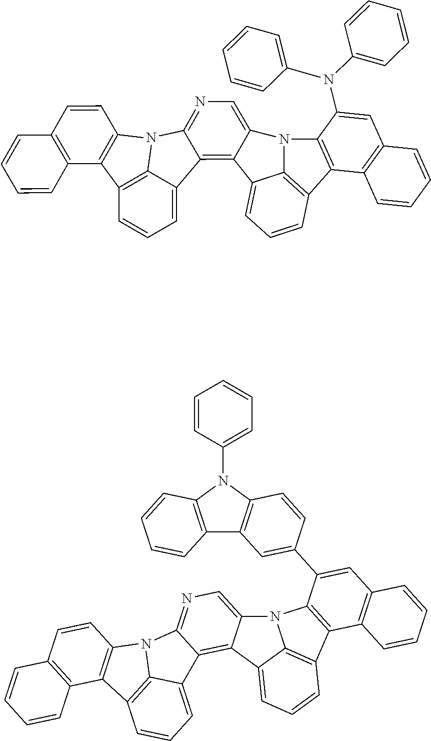 [SMILES: c1ccc(N(c2ccccc2)c2cc3ccccc3c3c4cccc5c6c7c8cccc9c%10c%11ccccc%11ccc%10n(c7ncc6n(c23)c54)c98)cc1 ; c1ccc(-n2c3ccccc3c3cc(-c4cc5ccccc5c5c6cccc7c8c9c%10cccc%11c%12c%13ccccc%13ccc%12n(c9ncc8n(c45)c76)c%11%10)ccc32)cc1]

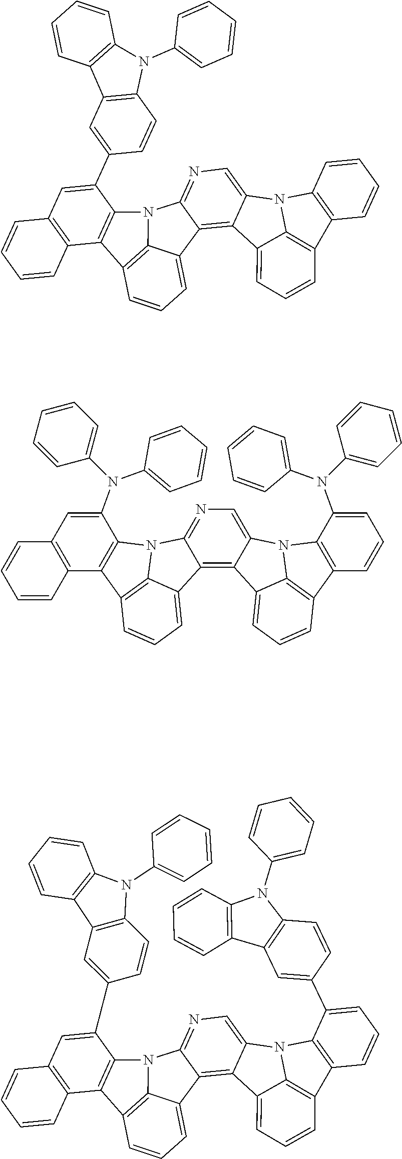 [SMILES: c1ccc(-n2c3ccccc3c3cc(-c4cc5ccccc5c5c6cccc7c8c9c%10cccc%11c%12ccccc%12n(c9cnc8n(c45)c76)c%11%10)ccc32)cc1 ; c1ccc(N(c2ccccc2)c2cccc3c4cccc5c6c7c8cccc9c%10c%11ccccc%11cc(N(c%11ccccc%11)c%11ccccc%11)c%10n(c7ncc6n(c23)c45)c89)cc1 ; c1ccc(-n2c3ccccc3c3cc(-c4cccc5c6cccc7c8c9c%10cccc%11c%12c%13ccccc%13cc(-c%13ccc%14c(c%13)c%13ccccc%13n%14-c%13ccccc%13)c%12n(c9ncc8n(c45)c67)c%10%11)ccc32)cc1]

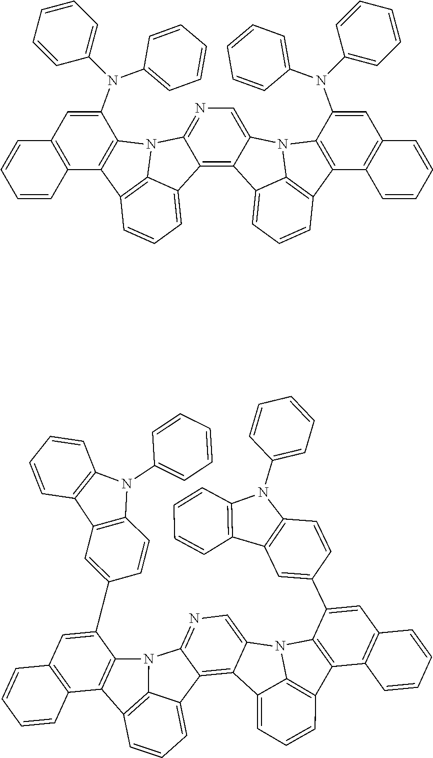 [SMILES: c1ccc(N(c2ccccc2)c2cc3ccccc3c3c4cccc5c6c7c8cccc9c%10c%11ccccc%11cc(N(c%11ccccc%11)c%11ccccc%11)c%10n(c7ncc6n(c23)c54)c89)cc1 ; c1ccc(-n2c3ccccc3c3cc(-c4cc5ccccc5c5c6cccc7c8c9c%10cccc%11c%12c%13ccccc%13cc(-c%13ccc%14c(c%13)c%13ccccc%13n%14-c%13ccccc%13)c%12n(c9ncc8n(c45)c76)c%10%11)ccc32)cc1]

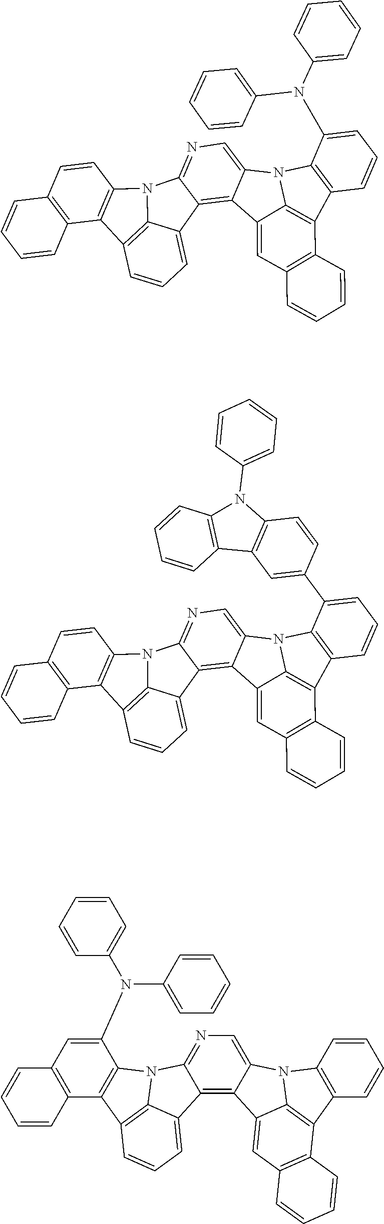 [SMILES: c1ccc(N(c2ccccc2)c2cccc3c4c5ccccc5cc5c6c7c8cccc9c%10c%11ccccc%11ccc%10n(c7ncc6n(c23)c54)c98)cc1 ; c1ccc(-n2c3ccccc3c3cc(-c4cccc5c6c7ccccc7cc7c8c9c%10cccc%11c%12c%13ccccc%13ccc%12n(c9ncc8n(c45)c76)c%11%10)ccc32)cc1 ; c1ccc(N(c2ccccc2)c2cc3ccccc3c3c4cccc5c6c7c8cc9ccccc9c9c%10ccccc%10n(c7cnc6n(c23)c54)c89)cc1]

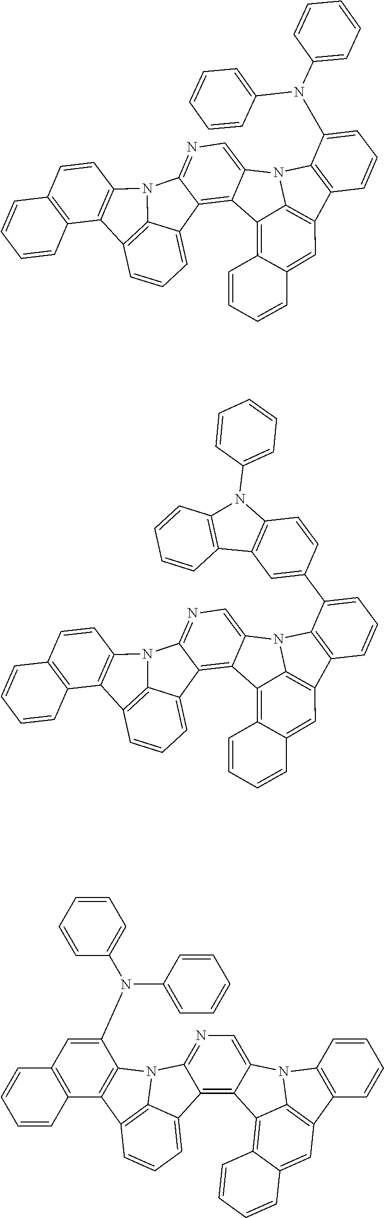 [SMILES: c1ccc(N(c2ccccc2)c2cccc3c4cc5ccccc5c5c6c7c8cccc9c%10c%11ccccc%11ccc%10n(c7ncc6n(c23)c45)c98)cc1 ; c1ccc(-n2c3ccccc3c3cc(-c4cccc5c6cc7ccccc7c7c8c9c%10cccc%11c%12c%13ccccc%13ccc%12n(c9ncc8n(c45)c67)c%11%10)ccc32)cc1 ; c1ccc(N(c2ccccc2)c2cc3ccccc3c3c4cccc5c6c7c8c9ccccc9cc9c%10ccccc%10n(c7cnc6n(c23)c54)c98)cc1]

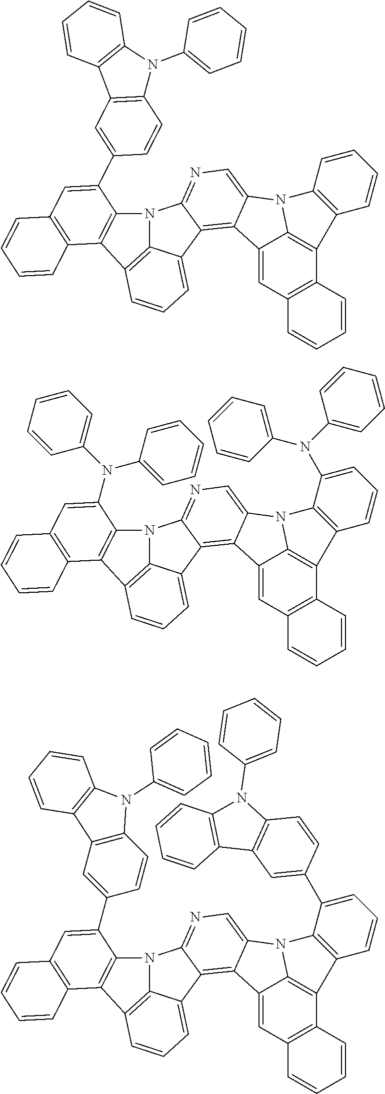

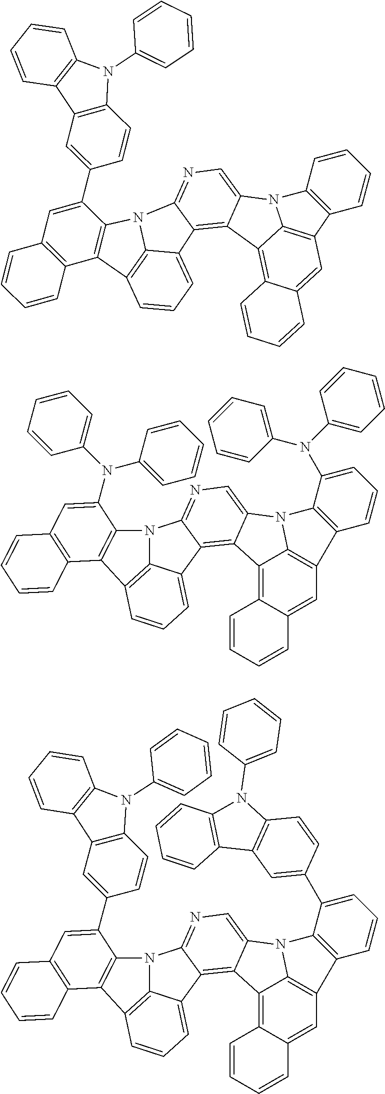

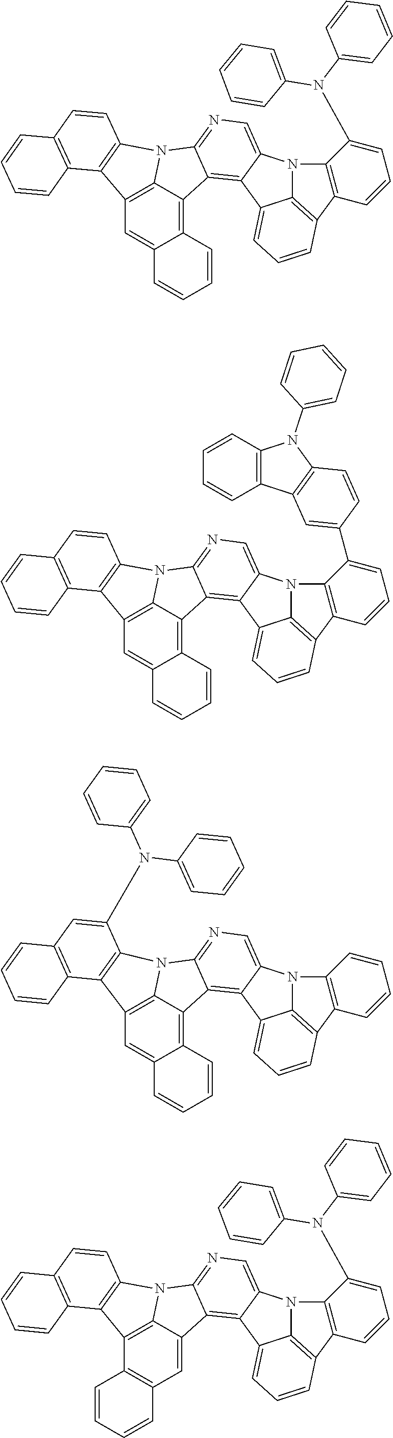 [SMILES: c1ccc(N(c2ccccc2)c2cccc3c4cccc5c6c7c8c9ccccc9cc9c%10c%11ccccc%11ccc%10n(c7ncc6n(c23)c45)c98)cc1 ; c1ccc(-n2c3ccccc3c3cc(-c4cccc5c6cccc7c8c9c%10c%11ccccc%11cc%11c%12c%13ccccc%13ccc%12n(c9ncc8n(c45)c67)c%11%10)ccc32)cc1 ; c1ccc(N(c2ccccc2)c2cc3ccccc3c3c4cc5ccccc5c5c6c7c8cccc9c%10ccccc%10n(c7cnc6n(c23)c45)c98)cc1 ; c1ccc(N(c2ccccc2)c2cccc3c4cccc5c6c7c8cc9ccccc9c9c%10c%11ccccc%11ccc%10n(c7ncc6n(c23)c45)c89)cc1]

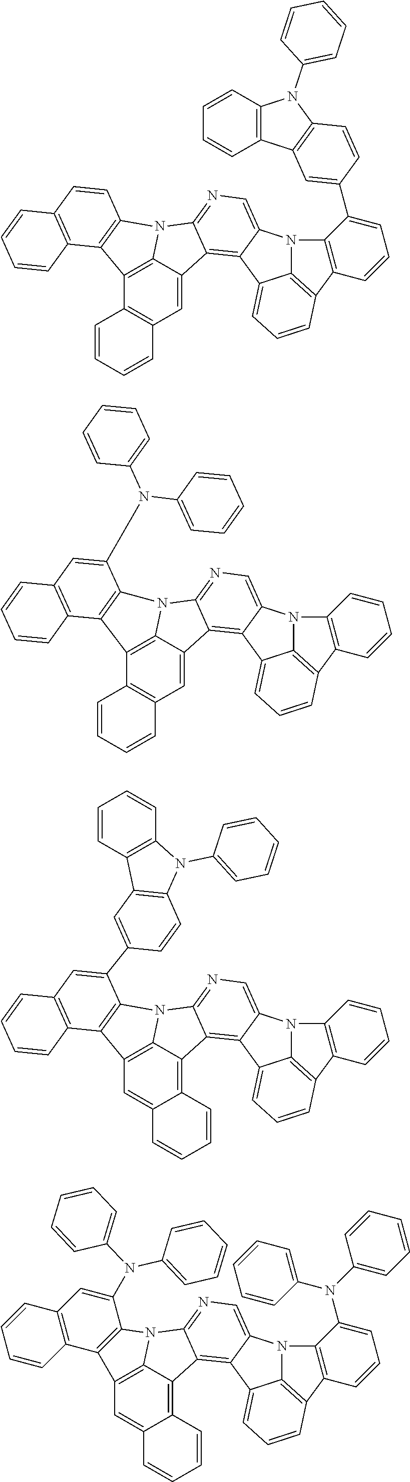

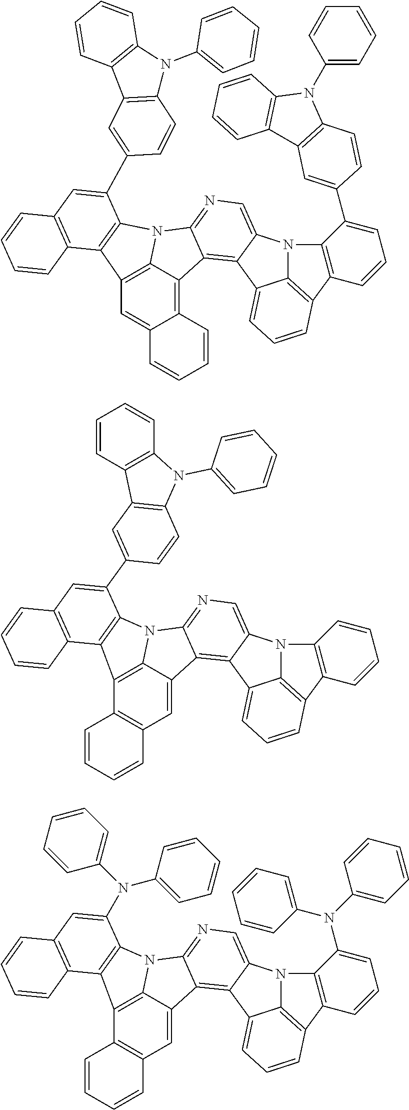

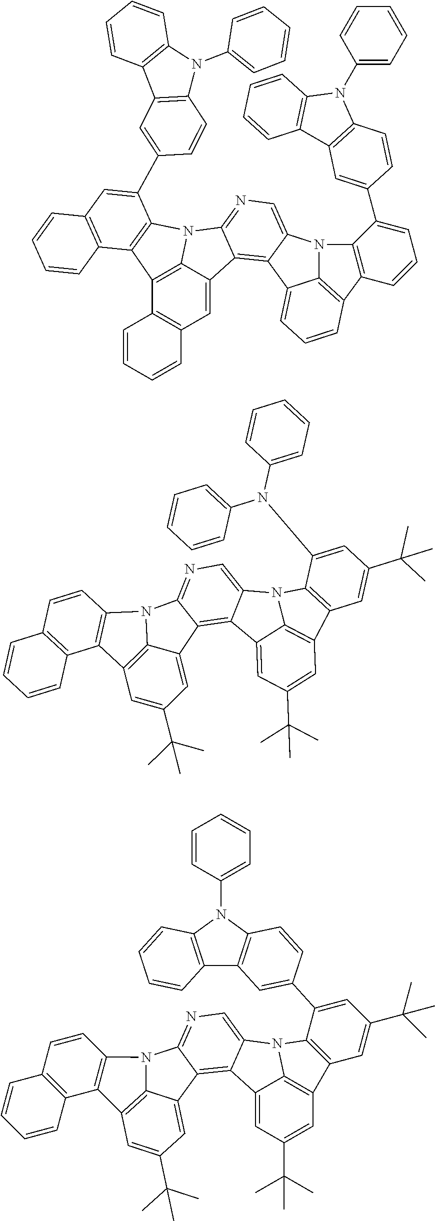 [SMILES: c1ccc(-n2c3ccccc3c3cc(-c4cccc5c6cccc7c8c9c%10cc%11ccccc%11c%11c%12c%13ccccc%13cc(-c%13ccc%14c(c%13)c%13ccccc%13n%14-c%13ccccc%13)c%12n(c9ncc8n(c45)c67)c%10%11)ccc32)cc1 ; CC(C)(C)c1cc(N(c2ccccc2)c2ccccc2)c2c(c1)c1cc(C(C)(C)C)cc3c4c5c6cc(C(C)(C)C)cc7c8c9ccccc9ccc8n(c5ncc4n2c13)c76 ; CC(C)(C)c1cc(-c2ccc3c(c2)c2ccccc2n3-c2ccccc2)c2c(c1)c1cc(C(C)(C)C)cc3c4c5c6cc(C(C)(C)C)cc7c8c9ccccc9ccc8n(c5ncc4n2c13)c76]

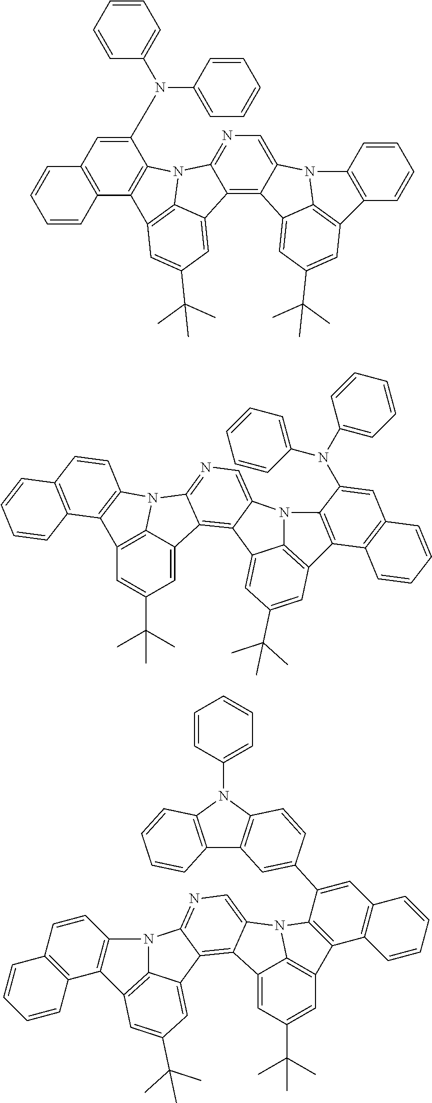

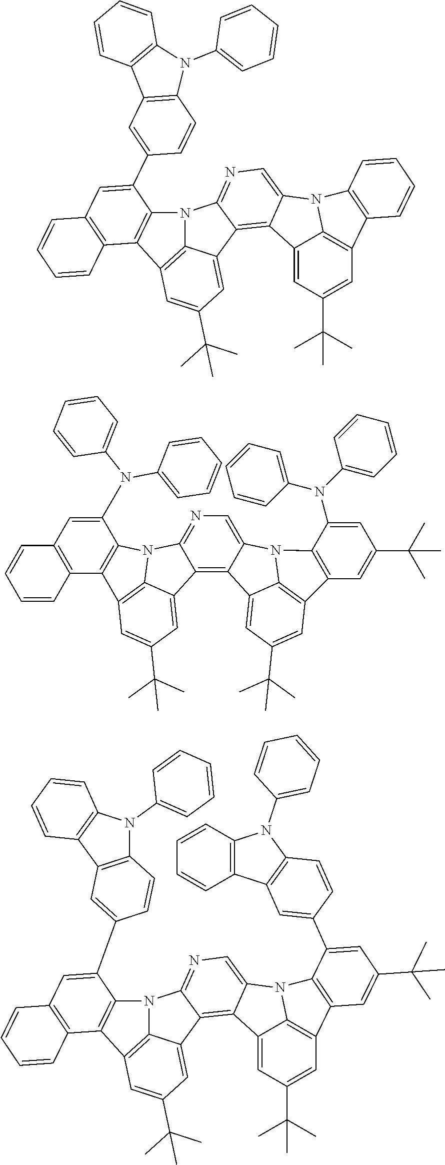

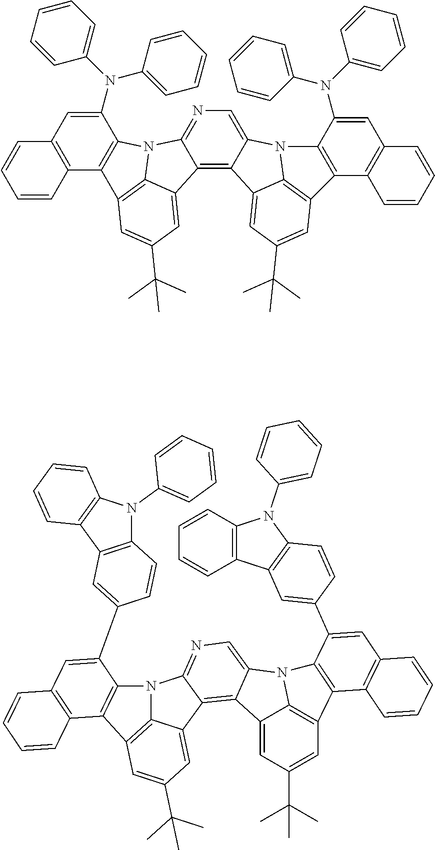 [SMILES: CC(C)(C)c1cc2c3c4c5cc(C(C)(C)C)cc6c7c8ccccc8cc(N(c8ccccc8)c8ccccc8)c7n(c4ncc3n3c4c(N(c7ccccc7)c7ccccc7)cc7ccccc7c4c(c1)c23)c56 ; CC(C)(C)c1cc2c3c4c5cc(C(C)(C)C)cc6c7c8ccccc8cc(-c8ccc9c(c8)c8ccccc8n9-c8ccccc8)c7n(c4ncc3n3c4c(-c7ccc8c(c7)c7ccccc7n8-c7ccccc7)cc7ccccc7c4c(c1)c23)c56]

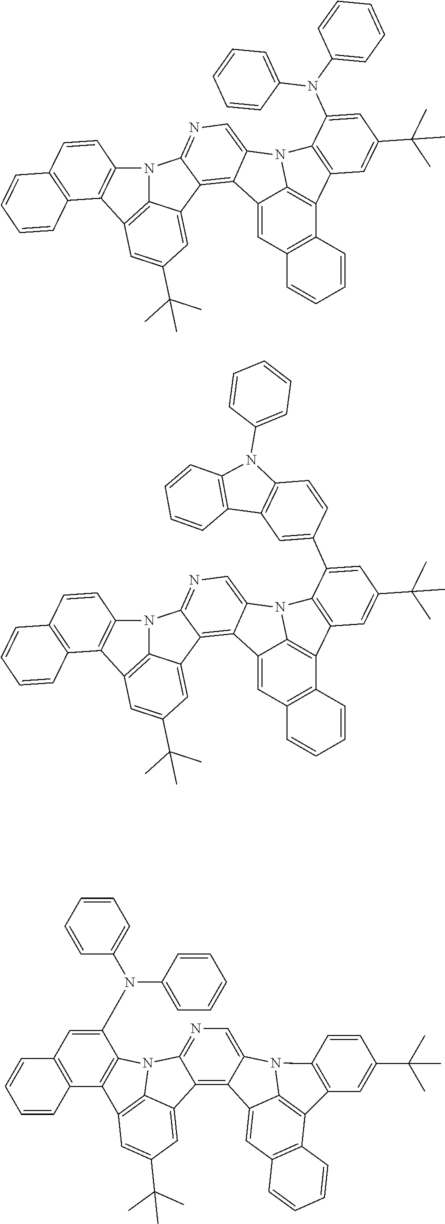 [SMILES: CC(C)(C)c1cc(N(c2ccccc2)c2ccccc2)c2c(c1)c1c3ccccc3cc3c4c5c6cc(C(C)(C)C)cc7c8c9ccccc9ccc8n(c5ncc4n2c31)c76 ; CC(C)(C)c1cc(-c2ccc3c(c2)c2ccccc2n3-c2ccccc2)c2c(c1)c1c3ccccc3cc3c4c5c6cc(C(C)(C)C)cc7c8c9ccccc9ccc8n(c5ncc4n2c31)c76 ; CC(C)(C)c1ccc2c(c1)c1c3ccccc3cc3c4c5c6cc(C(C)(C)C)cc7c8c9ccccc9cc(N(c9ccccc9)c9ccccc9)c8n(c5ncc4n2c31)c67]

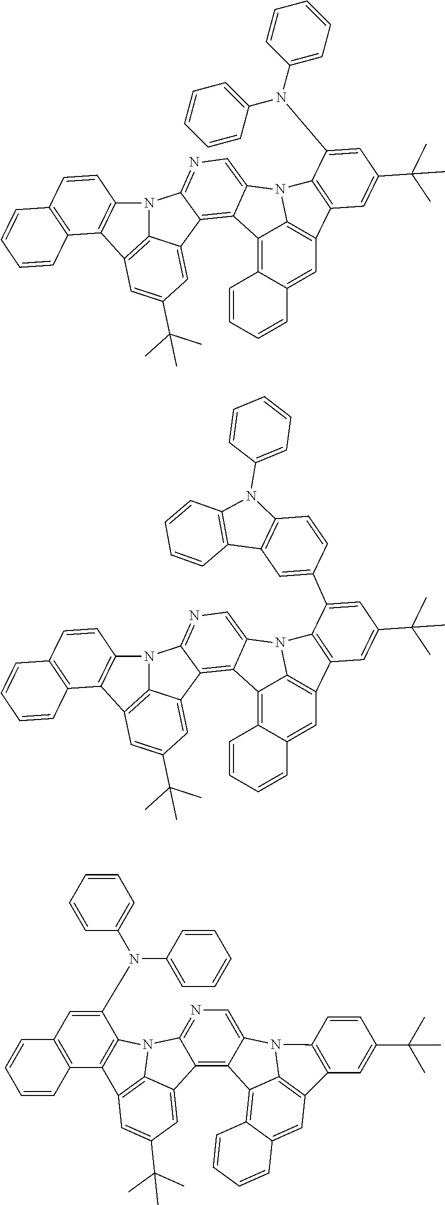 [SMILES: CC(C)(C)c1cc(N(c2ccccc2)c2ccccc2)c2c(c1)c1cc3ccccc3c3c4c5c6cc(C(C)(C)C)cc7c8c9ccccc9ccc8n(c5ncc4n2c13)c76 ; CC(C)(C)c1cc(-c2ccc3c(c2)c2ccccc2n3-c2ccccc2)c2c(c1)c1cc3ccccc3c3c4c5c6cc(C(C)(C)C)cc7c8c9ccccc9ccc8n(c5ncc4n2c13)c76 ; CC(C)(C)c1ccc2c(c1)c1cc3ccccc3c3c4c5c6cc(C(C)(C)C)cc7c8c9ccccc9cc(N(c9ccccc9)c9ccccc9)c8n(c5ncc4n2c13)c67]

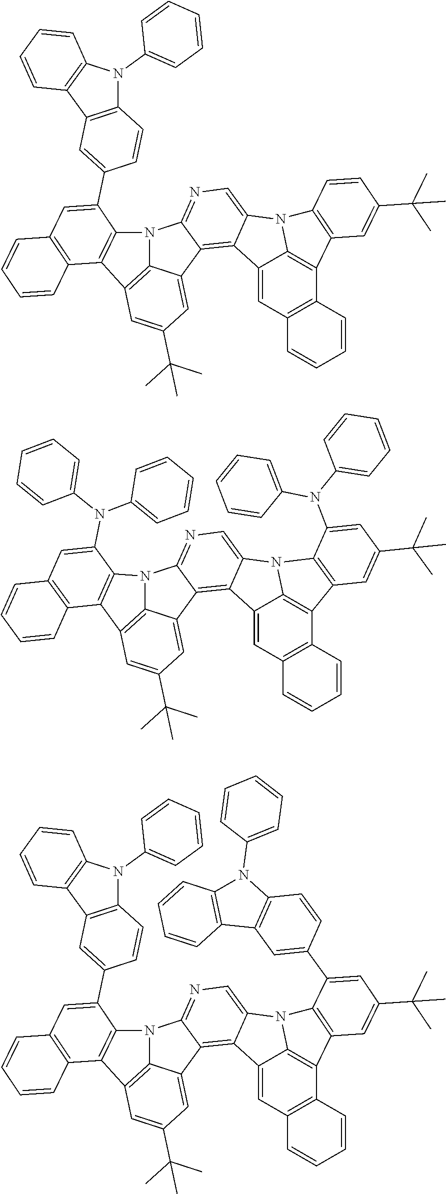 [SMILES: CC(C)(C)c1ccc2c(c1)c1c3ccccc3cc3c4c5c6cc(C(C)(C)C)cc7c8c9ccccc9cc(-c9ccc%10c(c9)c9ccccc9n%10-c9ccccc9)c8n(c5ncc4n2c31)c67 ; CC(C)(C)c1cc(N(c2ccccc2)c2ccccc2)c2c(c1)c1c3ccccc3cc3c4c5c6cc(C(C)(C)C)cc7c8c9ccccc9cc(N(c9ccccc9)c9ccccc9)c8n(c5ncc4n2c31)c67 ; CC(C)(C)c1cc(-c2ccc3c(c2)c2ccccc2n3-c2ccccc2)c2c(c1)c1c3ccccc3cc3c4c5c6cc(C(C)(C)C)cc7c8c9ccccc9cc(-c9ccc%10c(c9)c9ccccc9n%10-c9ccccc9)c8n(c5ncc4n2c31)c67]

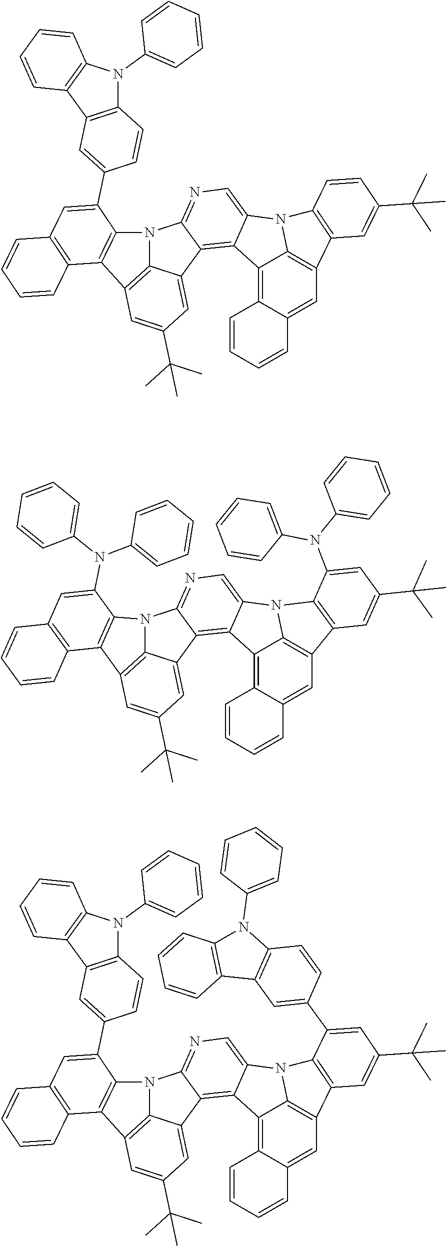 [SMILES: CC(C)(C)c1ccc2c(c1)c1cc3ccccc3c3c4c5c6cc(C(C)(C)C)cc7c8c9ccccc9cc(-c9ccc%10c(c9)c9ccccc9n%10-c9ccccc9)c8n(c5ncc4n2c13)c67 ; CC(C)(C)c1cc(N(c2ccccc2)c2ccccc2)c2c(c1)c1cc3ccccc3c3c4c5c6cc(C(C)(C)C)cc7c8c9ccccc9cc(N(c9ccccc9)c9ccccc9)c8n(c5ncc4n2c13)c67 ; CC(C)(C)c1cc(-c2ccc3c(c2)c2ccccc2n3-c2ccccc2)c2c(c1)c1cc3ccccc3c3c4c5c6cc(C(C)(C)C)cc7c8c9ccccc9cc(-c9ccc%10c(c9)c9ccccc9n%10-c9ccccc9)c8n(c5ncc4n2c13)c67]

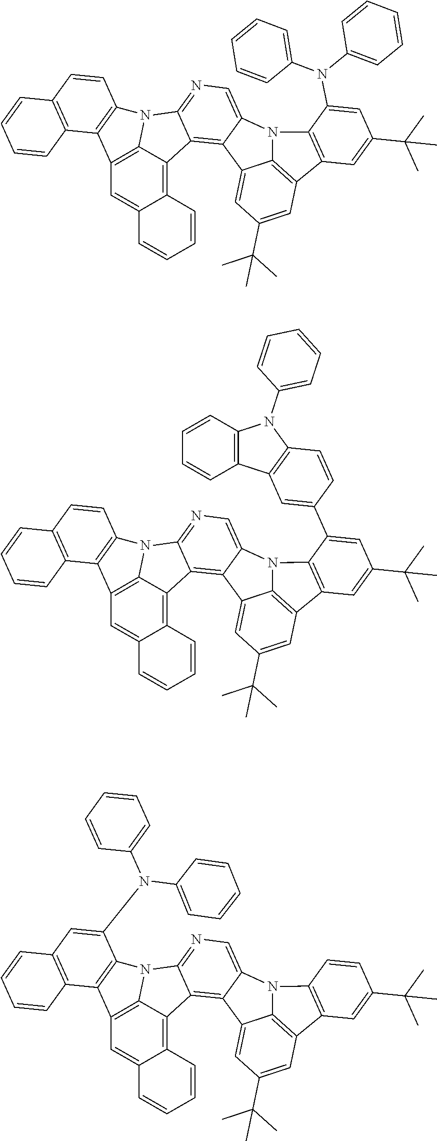 [SMILES: CC(C)(C)c1cc(N(c2ccccc2)c2ccccc2)c2c(c1)c1cc(C(C)(C)C)cc3c4c5c6c7ccccc7cc7c8c9ccccc9ccc8n(c5ncc4n2c13)c76 ; CC(C)(C)c1cc(-c2ccc3c(c2)c2ccccc2n3-c2ccccc2)c2c(c1)c1cc(C(C)(C)C)cc3c4c5c6c7ccccc7cc7c8c9ccccc9ccc8n(c5ncc4n2c13)c76 ; CC(C)(C)c1ccc2c(c1)c1cc(C(C)(C)C)cc3c4c5c6c7ccccc7cc7c8c9ccccc9cc(N(c9ccccc9)c9ccccc9)c8n(c5ncc4n2c13)c76]

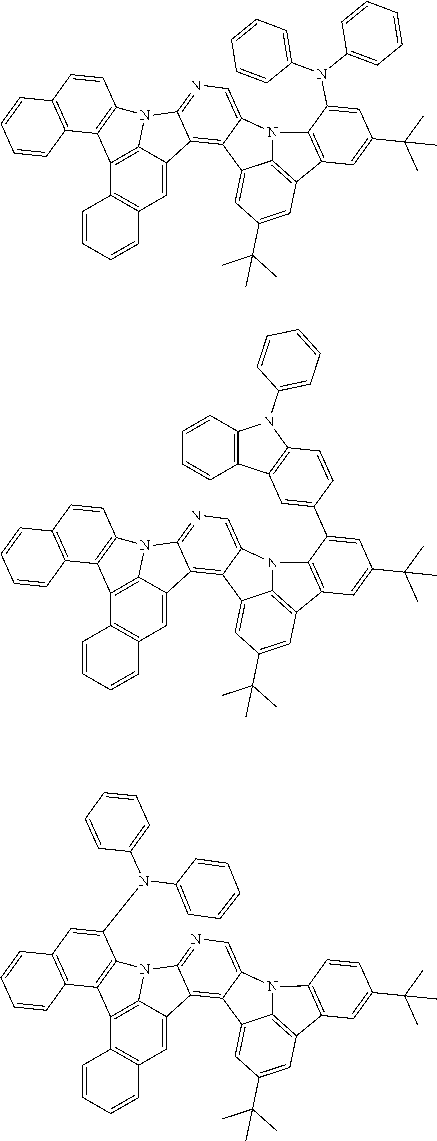 [SMILES: CC(C)(C)c1cc(N(c2ccccc2)c2ccccc2)c2c(c1)c1cc(C(C)(C)C)cc3c4c5c6cc7ccccc7c7c8c9ccccc9ccc8n(c5ncc4n2c13)c67 ; CC(C)(C)c1cc(-c2ccc3c(c2)c2ccccc2n3-c2ccccc2)c2c(c1)c1cc(C(C)(C)C)cc3c4c5c6cc7ccccc7c7c8c9ccccc9ccc8n(c5ncc4n2c13)c67 ; CC(C)(C)c1ccc2c(c1)c1cc(C(C)(C)C)cc3c4c5c6cc7ccccc7c7c8c9ccccc9cc(N(c9ccccc9)c9ccccc9)c8n(c5ncc4n2c13)c67]

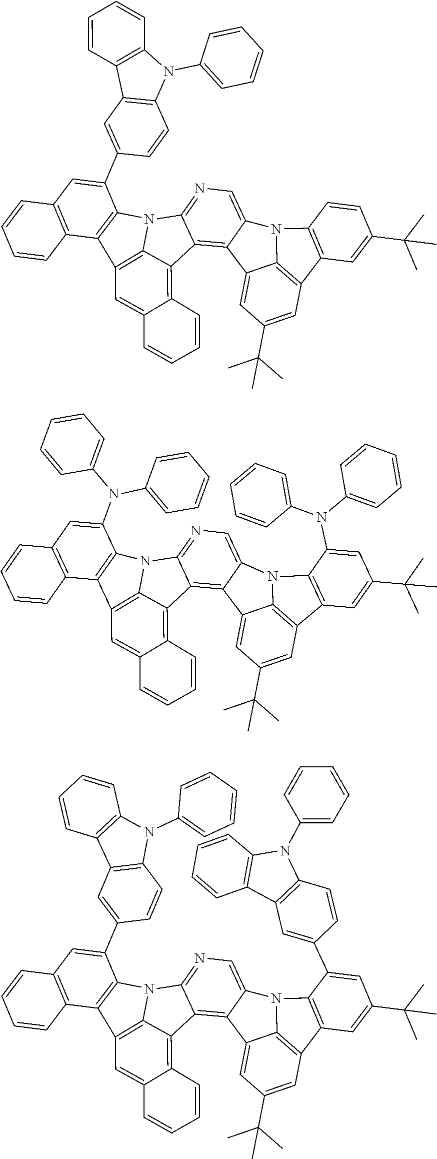 [SMILES: CC(C)(C)c1ccc2c(c1)c1cc(C(C)(C)C)cc3c4c5c6c7ccccc7cc7c8c9ccccc9cc(-c9ccc%10c(c9)c9ccccc9n%10-c9ccccc9)c8n(c5ncc4n2c13)c76 ; CC(C)(C)c1cc(N(c2ccccc2)c2ccccc2)c2c(c1)c1cc(C(C)(C)C)cc3c4c5c6c7ccccc7cc7c8c9ccccc9cc(N(c9ccccc9)c9ccccc9)c8n(c5ncc4n2c13)c76 ; CC(C)(C)c1cc(-c2ccc3c(c2)c2ccccc2n3-c2ccccc2)c2c(c1)c1cc(C(C)(C)C)cc3c4c5c6c7ccccc7cc7c8c9ccccc9cc(-c9ccc%10c(c9)c9ccccc9n%10-c9ccccc9)c8n(c5ncc4n2c13)c76]

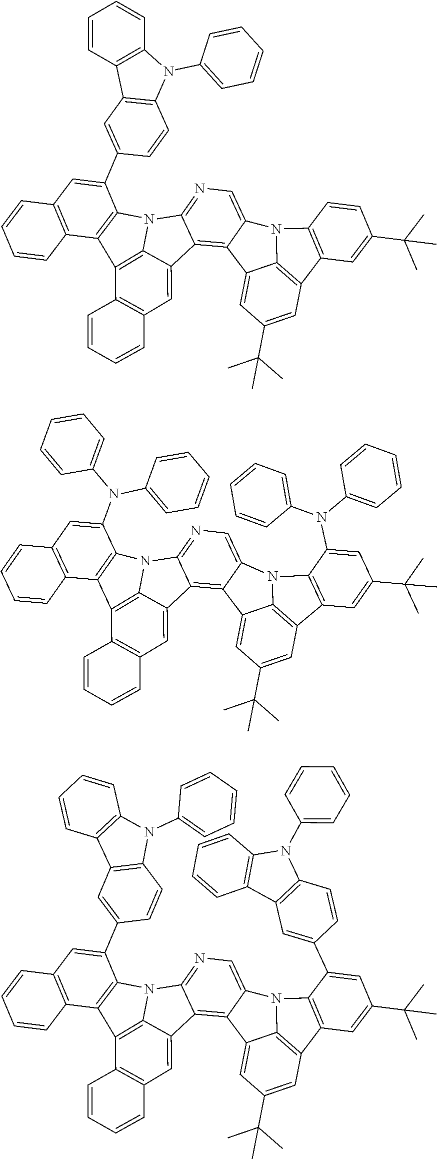 [SMILES: CC(C)(C)c1ccc2c(c1)c1cc(C(C)(C)C)cc3c4c5c6cc7ccccc7c7c8c9ccccc9cc(-c9ccc%10c(c9)c9ccccc9n%10-c9ccccc9)c8n(c5ncc4n2c13)c67 ; CC(C)(C)c1cc(N(c2ccccc2)c2ccccc2)c2c(c1)c1cc(C(C)(C)C)cc3c4c5c6cc7ccccc7c7c8c9ccccc9cc(N(c9ccccc9)c9ccccc9)c8n(c5ncc4n2c13)c67 ; CC(C)(C)c1cc(-c2ccc3c(c2)c2ccccc2n3-c2ccccc2)c2c(c1)c1cc(C(C)(C)C)cc3c4c5c6cc7ccccc7c7c8c9ccccc9cc(-c9ccc%10c(c9)c9ccccc9n%10-c9ccccc9)c8n(c5ncc4n2c13)c67]

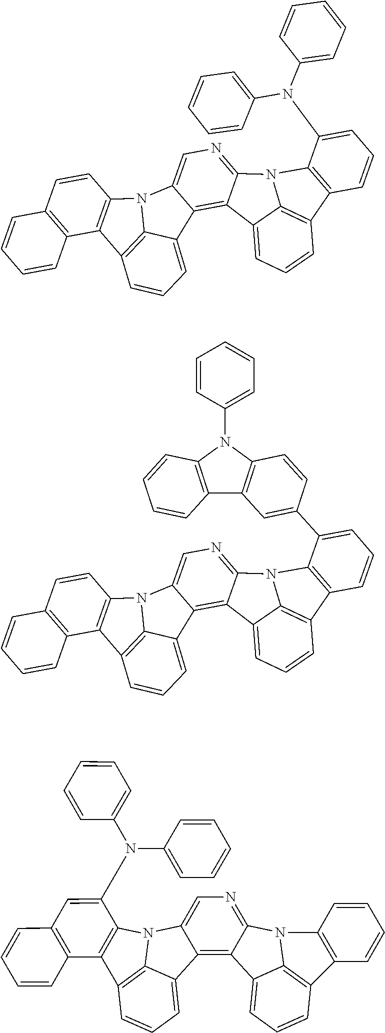 [SMILES: c1ccc(N(c2ccccc2)c2cccc3c4cccc5c6c7c8cccc9c%10c%11ccccc%11ccc%10n(c7cnc6n(c23)c45)c98)cc1 ; c1ccc(-n2c3ccccc3c3cc(-c4cccc5c6cccc7c8c9c%10cccc%11c%12c%13ccccc%13ccc%12n(c9cnc8n(c45)c67)c%11%10)ccc32)cc1 ; c1ccc(N(c2ccccc2)c2cc3ccccc3c3c4cccc5c6c7c8cccc9c%10ccccc%10n(c7ncc6n(c23)c54)c98)cc1]

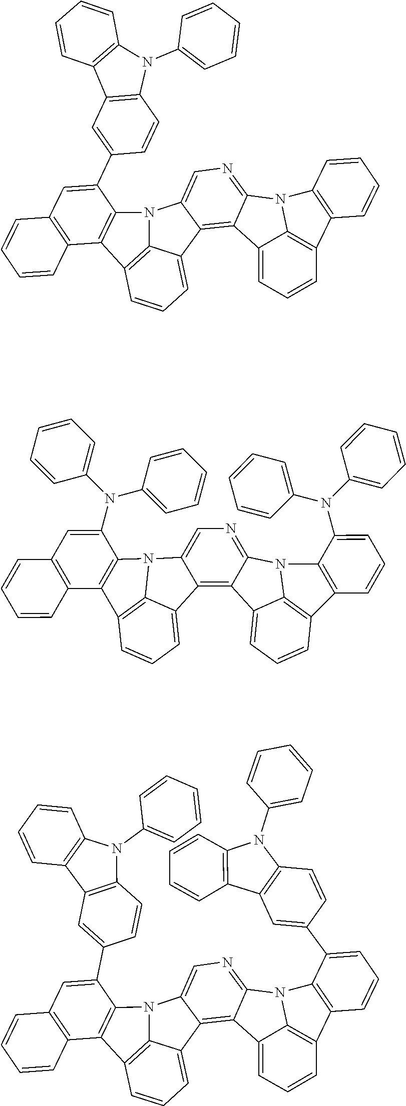 [SMILES: c1ccc(-n2c3ccccc3c3cc(-c4cc5ccccc5c5c6cccc7c8c9c%10cccc%11c%12ccccc%12n(c9ncc8n(c45)c76)c%11%10)ccc32)cc1 ; c1ccc(N(c2ccccc2)c2cc3ccccc3c3c4cccc5c6c7c8cccc9c%10cccc(N(c%11ccccc%11)c%11ccccc%11)c%10n(c7ncc6n(c23)c54)c98)cc1 ; c1ccc(-n2c3ccccc3c3cc(-c4cc5ccccc5c5c6cccc7c8c9c%10cccc%11c%12cccc(-c%13ccc%14c(c%13)c%13ccccc%13n%14-c%13ccccc%13)c%12n(c9ncc8n(c45)c76)c%11%10)ccc32)cc1]

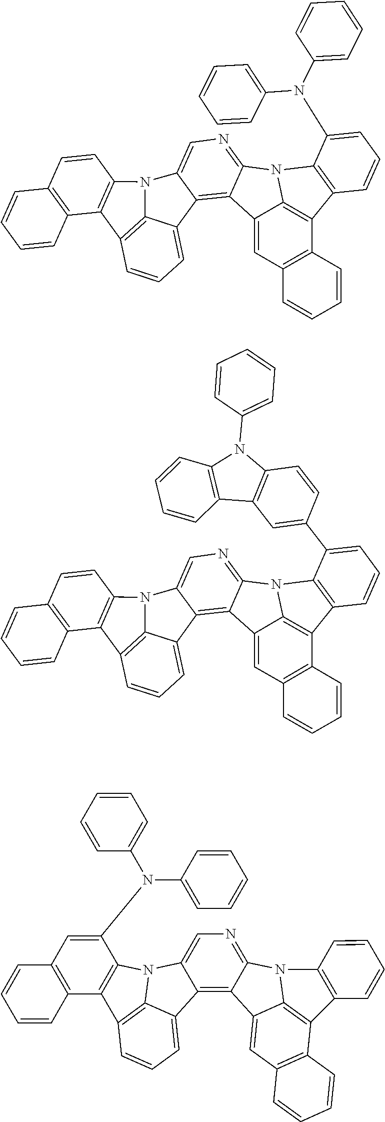 [SMILES: c1ccc(N(c2ccccc2)c2cccc3c4c5ccccc5cc5c6c7c8cccc9c%10c%11ccccc%11ccc%10n(c7cnc6n(c23)c54)c98)cc1 ; c1ccc(-n2c3ccccc3c3cc(-c4cccc5c6c7ccccc7cc7c8c9c%10cccc%11c%12c%13ccccc%13ccc%12n(c9cnc8n(c45)c76)c%11%10)ccc32)cc1 ; c1ccc(N(c2ccccc2)c2cc3ccccc3c3c4cccc5c6c7c8cc9ccccc9c9c%10ccccc%10n(c7ncc6n(c23)c54)c89)cc1]

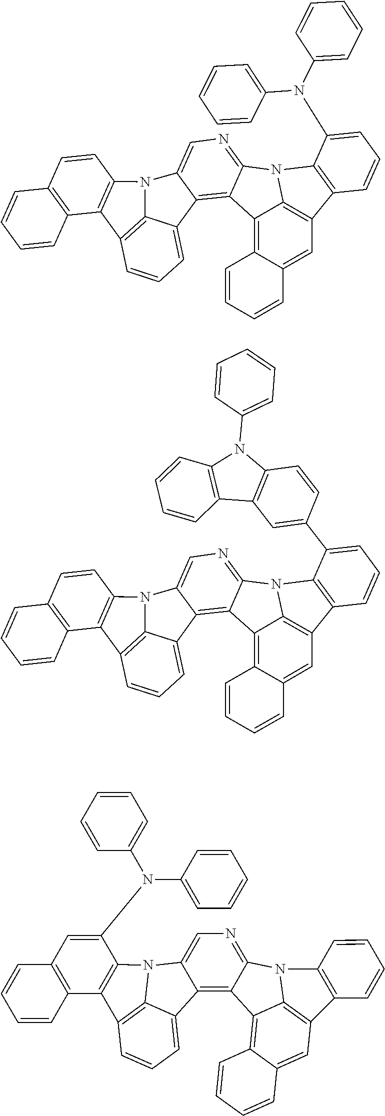

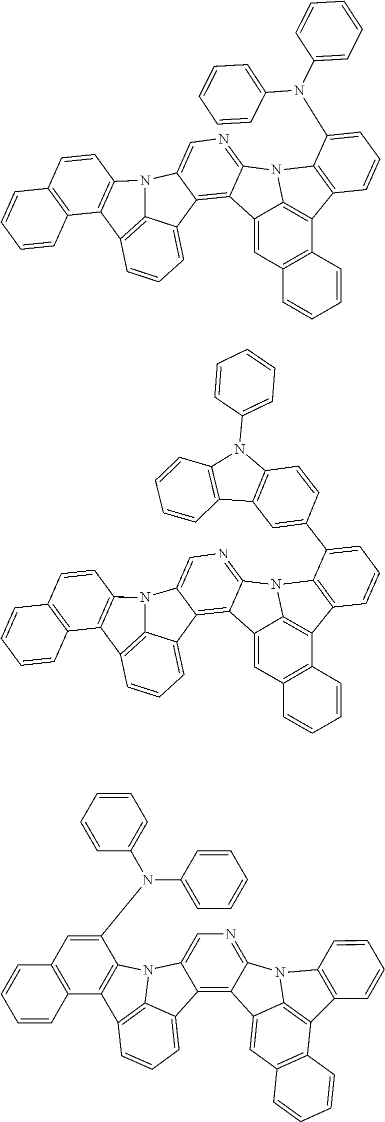 [SMILES: c1ccc(N(c2ccccc2)c2cccc3c4c5ccccc5cc5c6c7c8cccc9c%10c%11ccccc%11ccc%10n(c7cnc6n(c23)c54)c98)cc1 ; c1ccc(-n2c3ccccc3c3cc(-c4cccc5c6c7ccccc7cc7c8c9c%10cccc%11c%12c%13ccccc%13ccc%12n(c9cnc8n(c45)c76)c%11%10)ccc32)cc1 ; c1ccc(N(c2ccccc2)c2cc3ccccc3c3c4cccc5c6c7c8cc9ccccc9c9c%10ccccc%10n(c7ncc6n(c23)c54)c89)cc1]

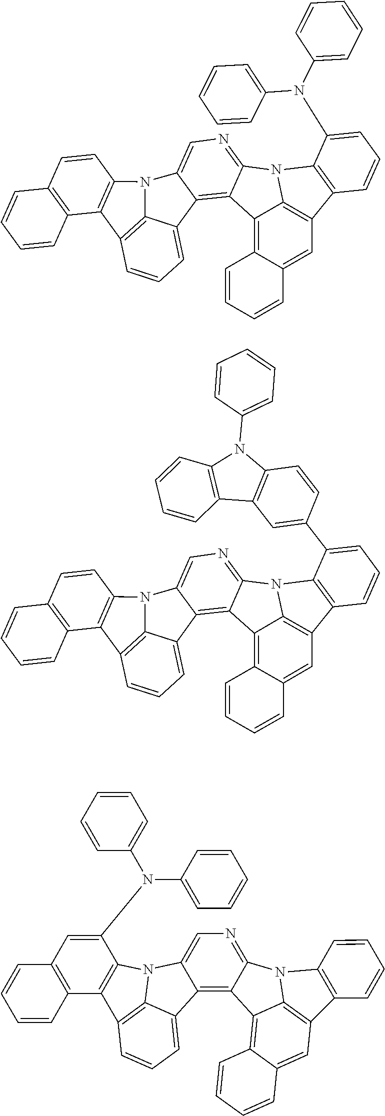

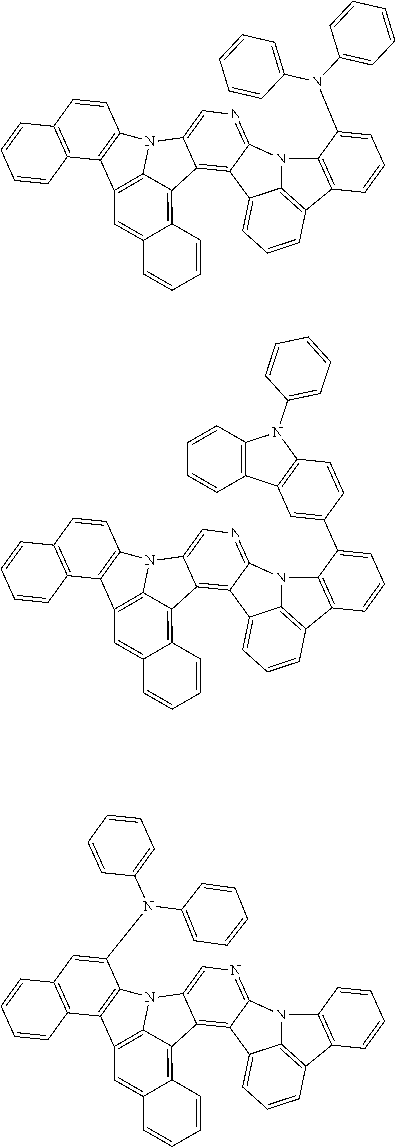 [SMILES: c1ccc(N(c2ccccc2)c2cccc3c4cccc5c6c7c8c9ccccc9cc9c%10c%11ccccc%11ccc%10n(c7cnc6n(c23)c45)c98)cc1 ; c1ccc(-n2c3ccccc3c3cc(-c4cccc5c6cccc7c8c9c%10c%11ccccc%11cc%11c%12c%13ccccc%13ccc%12n(c9cnc8n(c45)c67)c%11%10)ccc32)cc1 ; c1ccc(N(c2ccccc2)c2cc3ccccc3c3c4cc5ccccc5c5c6c7c8cccc9c%10ccccc%10n(c7ncc6n(c23)c45)c98)cc1]

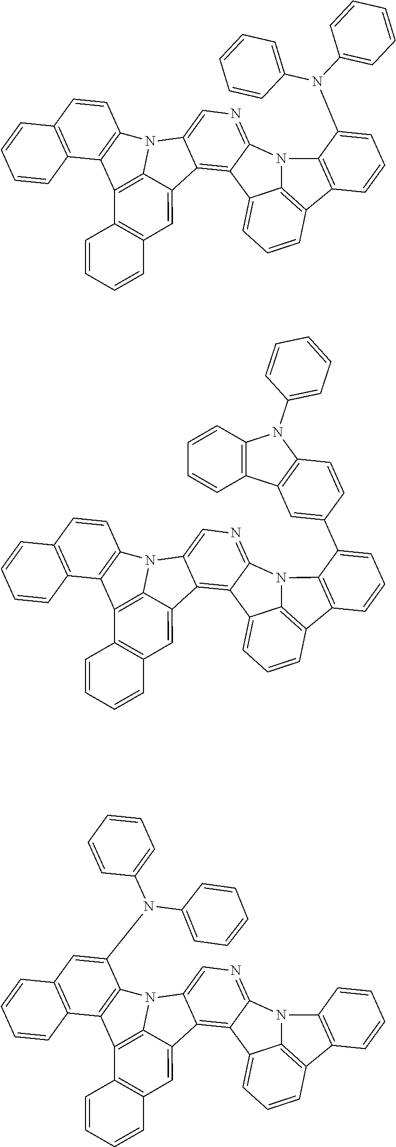 [SMILES: c1ccc(N(c2ccccc2)c2cccc3c4cccc5c6c7c8cc9ccccc9c9c%10c%11ccccc%11ccc%10n(c7cnc6n(c23)c45)c89)cc1 ; c1ccc(-n2c3ccccc3c3cc(-c4cccc5c6cccc7c8c9c%10cc%11ccccc%11c%11c%12c%13ccccc%13ccc%12n(c9cnc8n(c45)c67)c%10%11)ccc32)cc1 ; c1ccc(N(c2ccccc2)c2cc3ccccc3c3c4c5ccccc5cc5c6c7c8cccc9c%10ccccc%10n(c7ncc6n(c23)c54)c98)cc1]

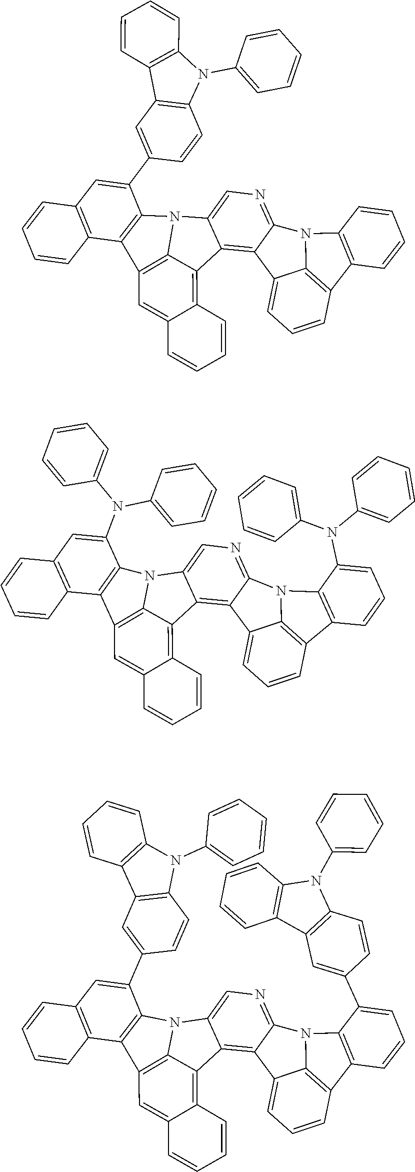 [SMILES: c1ccc(-n2c3ccccc3c3cc(-c4cc5ccccc5c5c6cc7ccccc7c7c8c9c%10cccc%11c%12ccccc%12n(c9ncc8n(c45)c67)c%11%10)ccc32)cc1 ; c1ccc(N(c2ccccc2)c2cc3ccccc3c3c4cc5ccccc5c5c6c7c8cccc9c%10cccc(N(c%11ccccc%11)c%11ccccc%11)c%10n(c7ncc6n(c23)c45)c98)cc1 ; c1ccc(-n2c3ccccc3c3cc(-c4cc5ccccc5c5c6cc7ccccc7c7c8c9c%10cccc%11c%12cccc(-c%13ccc%14c(c%13)c%13ccccc%13n%14-c%13ccccc%13)c%12n(c9ncc8n(c45)c67)c%11%10)ccc32)cc1]

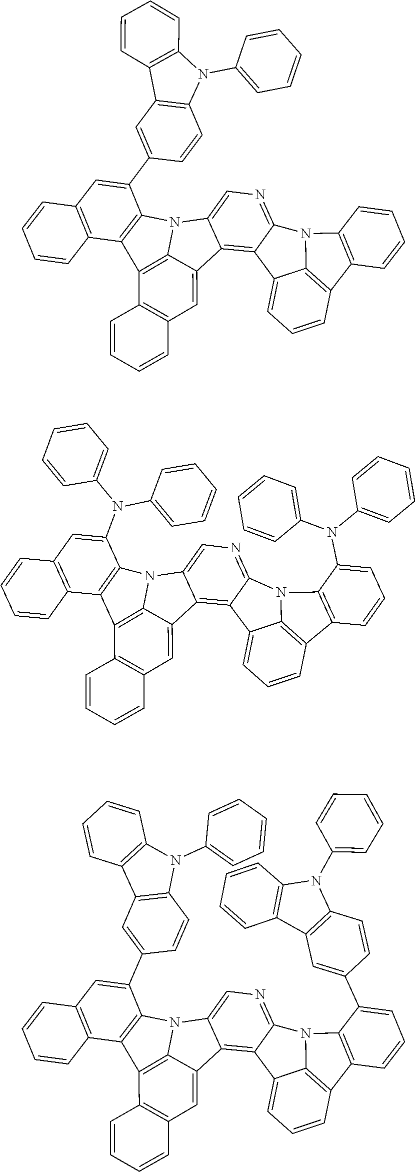 [SMILES: c1ccc(-n2c3ccccc3c3cc(-c4cc5ccccc5c5c6c7ccccc7cc7c8c9c%10cccc%11c%12ccccc%12n(c9ncc8n(c45)c76)c%11%10)ccc32)cc1 ; c1ccc(N(c2ccccc2)c2cc3ccccc3c3c4c5ccccc5cc5c6c7c8cccc9c%10cccc(N(c%11ccccc%11)c%11ccccc%11)c%10n(c7ncc6n(c23)c54)c98)cc1 ; c1ccc(-n2c3ccccc3c3cc(-c4cc5ccccc5c5c6c7ccccc7cc7c8c9c%10cccc%11c%12cccc(-c%13ccc%14c(c%13)c%13ccccc%13n%14-c%13ccccc%13)c%12n(c9ncc8n(c45)c76)c%11%10)ccc32)cc1]

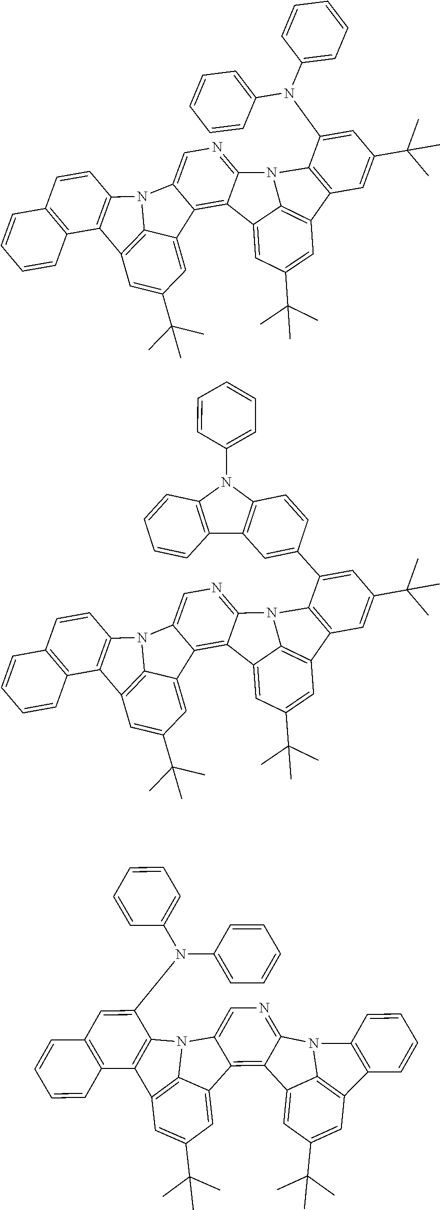

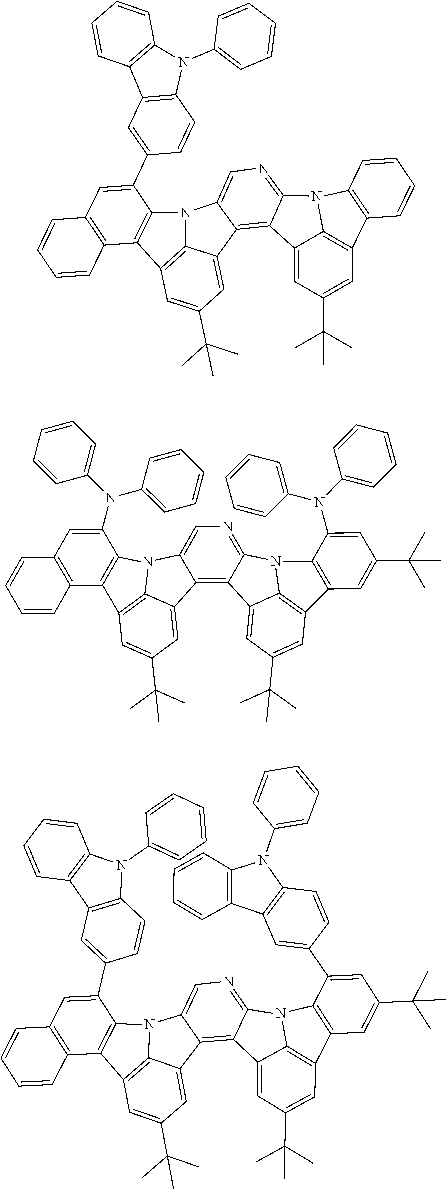 [SMILES: CC(C)(C)c1cc2c3ccccc3n3c4ncc5c(c6cc(C(C)(C)C)cc7c8c9ccccc9cc(-c9ccc%10c(c9)c9ccccc9n%10-c9ccccc9)c8n5c67)c4c(c1)c23 ; CC(C)(C)c1cc(N(c2ccccc2)c2ccccc2)c2c(c1)c1cc(C(C)(C)C)cc3c4c5c6cc(C(C)(C)C)cc7c8c9ccccc9cc(N(c9ccccc9)c9ccccc9)c8n(c5cnc4n2c13)c67 ; CC(C)(C)c1cc(-c2ccc3c(c2)c2ccccc2n3-c2ccccc2)c2c(c1)c1cc(C(C)(C)C)cc3c4c5c6cc(C(C)(C)C)cc7c8c9ccccc9cc(-c9ccc%10c(c9)c9ccccc9n%10-c9ccccc9)c8n(c5cnc4n2c13)c67]

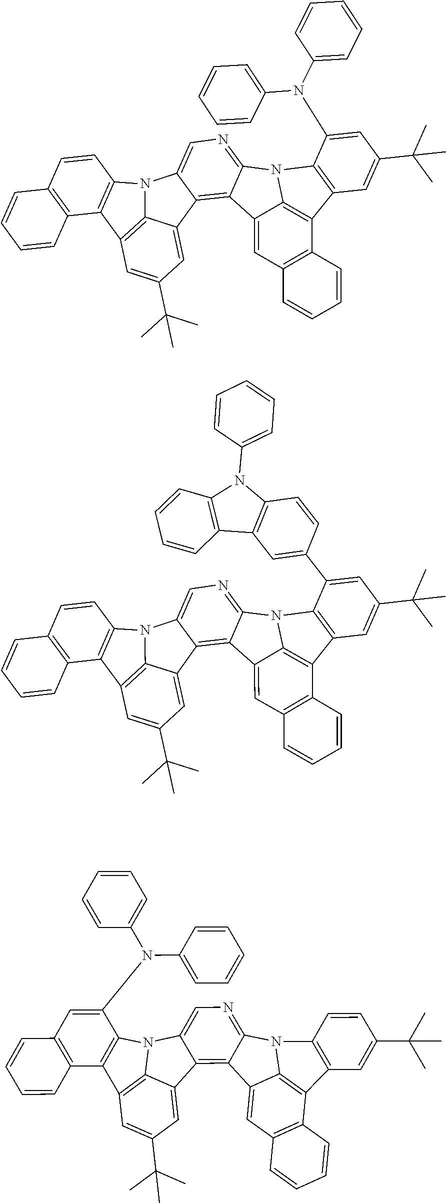 [SMILES: CC(C)(C)c1cc(N(c2ccccc2)c2ccccc2)c2c(c1)c1c3ccccc3cc3c4c5c6cc(C(C)(C)C)cc7c8c9ccccc9ccc8n(c5cnc4n2c31)c76 ; CC(C)(C)c1cc(-c2ccc3c(c2)c2ccccc2n3-c2ccccc2)c2c(c1)c1c3ccccc3cc3c4c5c6cc(C(C)(C)C)cc7c8c9ccccc9ccc8n(c5cnc4n2c31)c76 ; CC(C)(C)c1ccc2c(c1)c1c3ccccc3cc3c4c5c6cc(C(C)(C)C)cc7c8c9ccccc9cc(N(c9ccccc9)c9ccccc9)c8n(c5cnc4n2c31)c67]

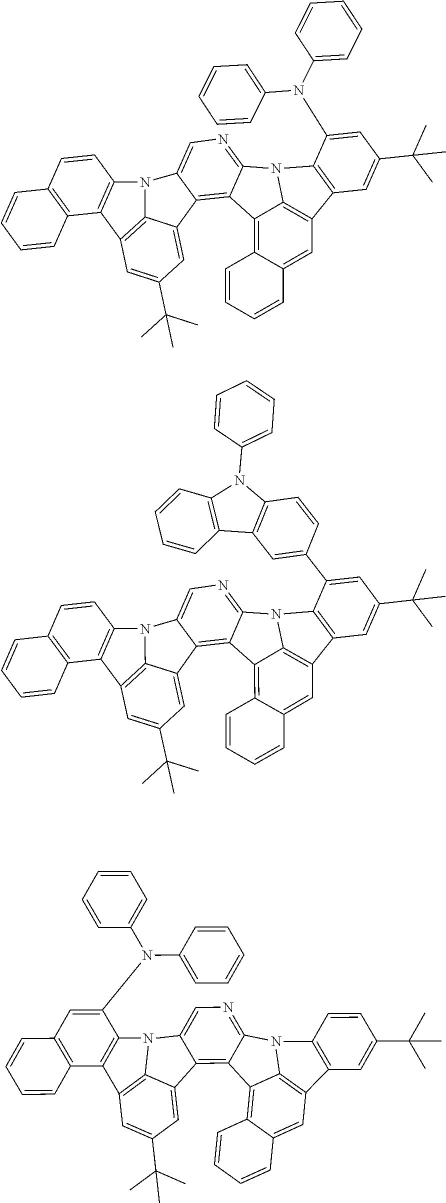 [SMILES: CC(C)(C)c1cc(N(c2ccccc2)c2ccccc2)c2c(c1)c1cc3ccccc3c3c4c5c6cc(C(C)(C)C)cc7c8c9ccccc9ccc8n(c5cnc4n2c13)c76 ; CC(C)(C)c1cc(-c2ccc3c(c2)c2ccccc2n3-c2ccccc2)c2c(c1)c1cc3ccccc3c3c4c5c6cc(C(C)(C)C)cc7c8c9ccccc9ccc8n(c5cnc4n2c13)c76 ; CC(C)(C)c1ccc2c(c1)c1cc3ccccc3c3c4c5c6cc(C(C)(C)C)cc7c8c9ccccc9cc(N(c9ccccc9)c9ccccc9)c8n(c5cnc4n2c13)c67]

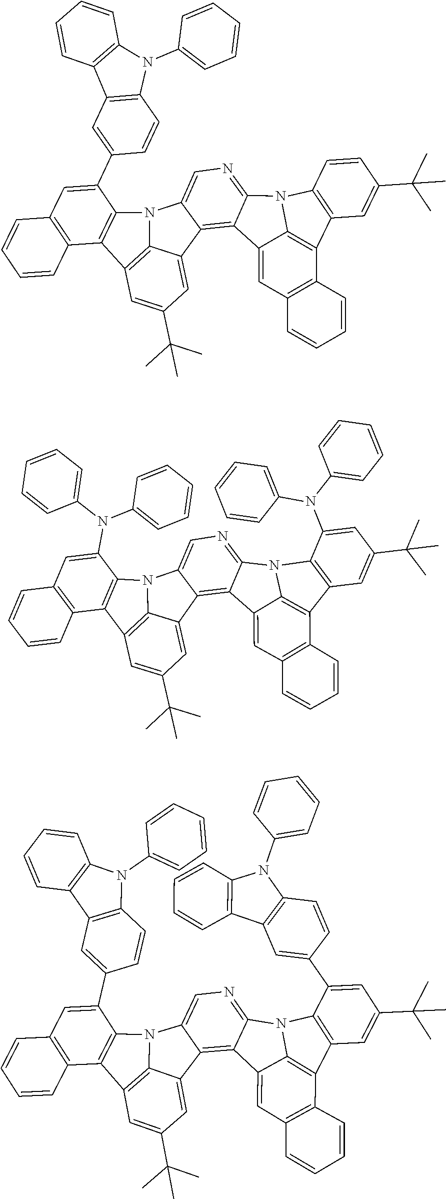 [SMILES: CC(C)(C)c1ccc2c(c1)c1c3ccccc3cc3c4c5c6cc(C(C)(C)C)cc7c8c9ccccc9cc(-c9ccc%10c(c9)c9ccccc9n%10-c9ccccc9)c8n(c5cnc4n2c31)c67 ; CC(C)(C)c1cc(N(c2ccccc2)c2ccccc2)c2c(c1)c1c3ccccc3cc3c4c5c6cc(C(C)(C)C)cc7c8c9ccccc9cc(N(c9ccccc9)c9ccccc9)c8n(c5cnc4n2c31)c67 ; CC(C)(C)c1cc(-c2ccc3c(c2)c2ccccc2n3-c2ccccc2)c2c(c1)c1c3ccccc3cc3c4c5c6cc(C(C)(C)C)cc7c8c9ccccc9cc(-c9ccc%10c(c9)c9ccccc9n%10-c9ccccc9)c8n(c5cnc4n2c31)c67]

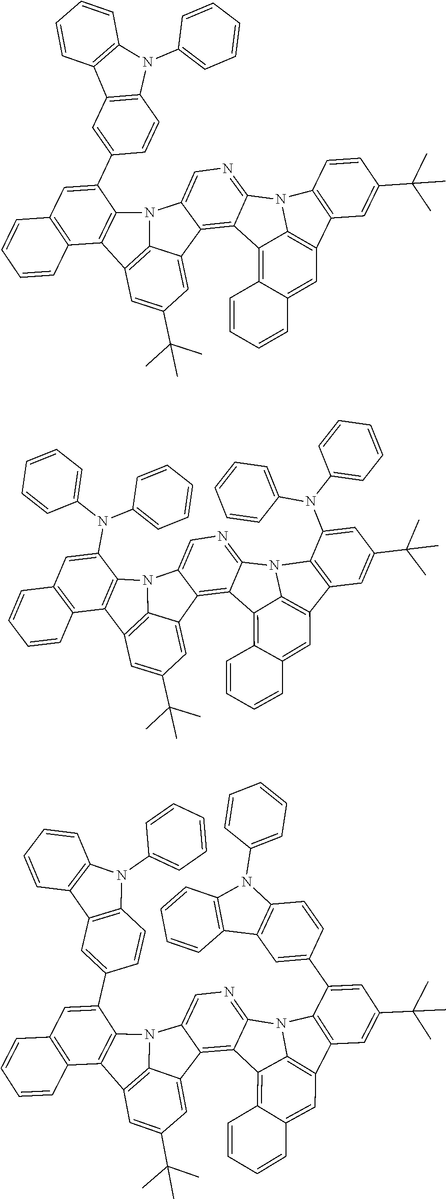 [SMILES: CC(C)(C)c1ccc2c(c1)c1cc3ccccc3c3c4c5c6cc(C(C)(C)C)cc7c8c9ccccc9cc(-c9ccc%10c(c9)c9ccccc9n%10-c9ccccc9)c8n(c5cnc4n2c13)c67 ; CC(C)(C)c1cc(N(c2ccccc2)c2ccccc2)c2c(c1)c1cc3ccccc3c3c4c5c6cc(C(C)(C)C)cc7c8c9ccccc9cc(N(c9ccccc9)c9ccccc9)c8n(c5cnc4n2c13)c67 ; CC(C)(C)c1cc(-c2ccc3c(c2)c2ccccc2n3-c2ccccc2)c2c(c1)c1cc3ccccc3c3c4c5c6cc(C(C)(C)C)cc7c8c9ccccc9cc(-c9ccc%10c(c9)c9ccccc9n%10-c9ccccc9)c8n(c5cnc4n2c13)c67]

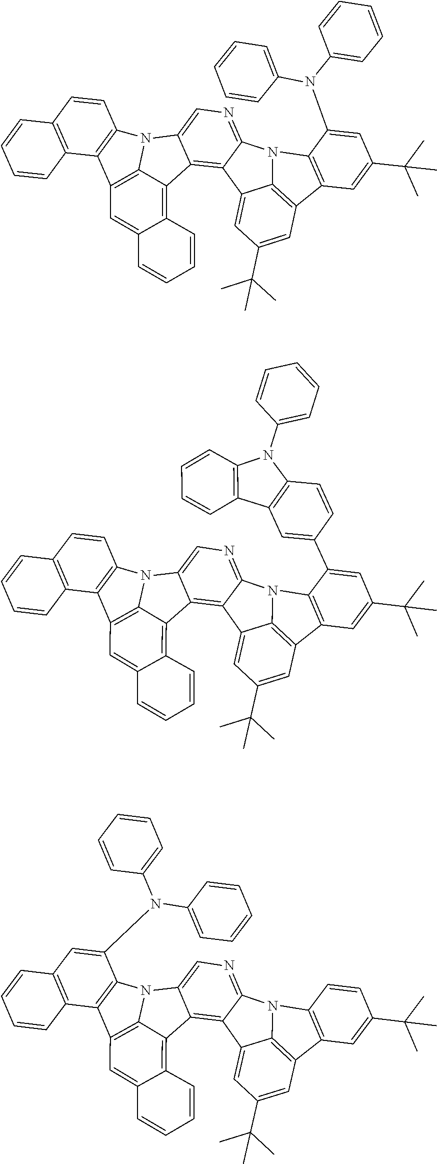 [SMILES: CC(C)(C)c1cc(N(c2ccccc2)c2ccccc2)c2c(c1)c1cc(C(C)(C)C)cc3c4c5c6c7ccccc7cc7c8c9ccccc9ccc8n(c5cnc4n2c13)c76 ; CC(C)(C)c1cc(-c2ccc3c(c2)c2ccccc2n3-c2ccccc2)c2c(c1)c1cc(C(C)(C)C)cc3c4c5c6c7ccccc7cc7c8c9ccccc9ccc8n(c5cnc4n2c13)c76 ; CC(C)(C)c1ccc2c(c1)c1cc(C(C)(C)C)cc3c4c5c6c7ccccc7cc7c8c9ccccc9cc(N(c9ccccc9)c9ccccc9)c8n(c5cnc4n2c13)c76]

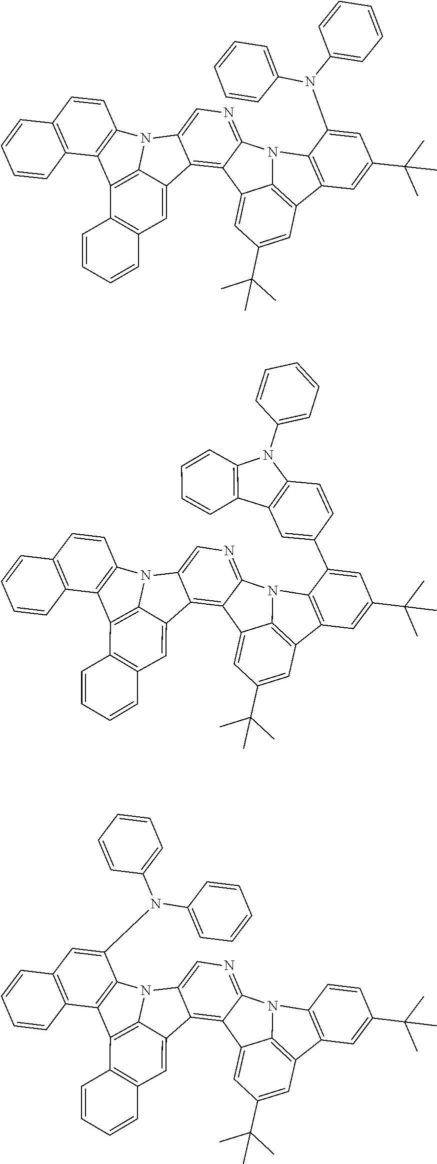 [SMILES: CC(C)(C)c1cc(N(c2ccccc2)c2ccccc2)c2c(c1)c1cc(C(C)(C)C)cc3c4c5c6cc7ccccc7c7c8c9ccccc9ccc8n(c5cnc4n2c13)c67 ; CC(C)(C)c1cc(-c2ccc3c(c2)c2ccccc2n3-c2ccccc2)c2c(c1)c1cc(C(C)(C)C)cc3c4c5c6cc7ccccc7c7c8c9ccccc9ccc8n(c5cnc4n2c13)c67 ; CC(C)(C)c1ccc2c(c1)c1cc(C(C)(C)C)cc3c4c5c6cc7ccccc7c7c8c9ccccc9cc(N(c9ccccc9)c9ccccc9)c8n(c5cnc4n2c13)c67]

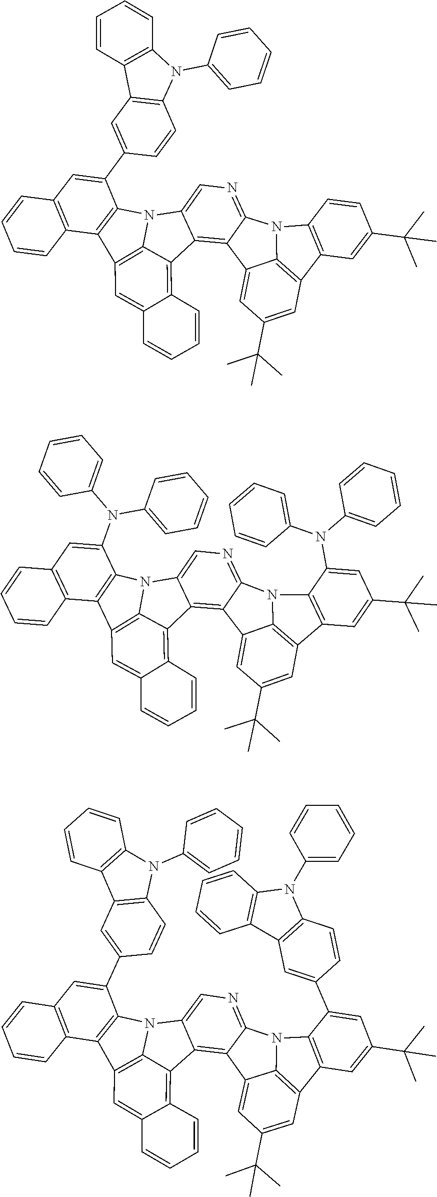 [SMILES: CC(C)(C)c1ccc2c(c1)c1cc(C(C)(C)C)cc3c4c5c6c7ccccc7cc7c8c9ccccc9cc(-c9ccc%10c(c9)c9ccccc9n%10-c9ccccc9)c8n(c5cnc4n2c13)c76 ; CC(C)(C)c1cc(N(c2ccccc2)c2ccccc2)c2c(c1)c1cc(C(C)(C)C)cc3c4c5c6c7ccccc7cc7c8c9ccccc9cc(N(c9ccccc9)c9ccccc9)c8n(c5cnc4n2c13)c76 ; CC(C)(C)c1cc(-c2ccc3c(c2)c2ccccc2n3-c2ccccc2)c2c(c1)c1cc(C(C)(C)C)cc3c4c5c6c7ccccc7cc7c8c9ccccc9cc(-c9ccc%10c(c9)c9ccccc9n%10-c9ccccc9)c8n(c5cnc4n2c13)c76]

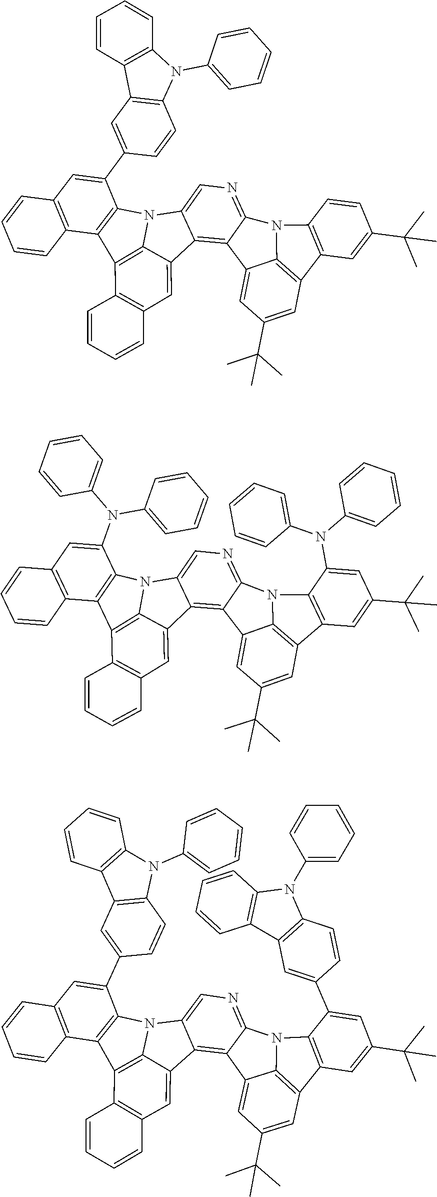 [SMILES: CC(C)(C)c1ccc2c(c1)c1cc(C(C)(C)C)cc3c4c5c6cc7ccccc7c7c8c9ccccc9cc(-c9ccc%10c(c9)c9ccccc9n%10-c9ccccc9)c8n(c5cnc4n2c13)c67 ; CC(C)(C)c1cc(N(c2ccccc2)c2ccccc2)c2c(c1)c1cc(C(C)(C)C)cc3c4c5c6cc7ccccc7c7c8c9ccccc9cc(N(c9ccccc9)c9ccccc9)c8n(c5cnc4n2c13)c67 ; CC(C)(C)c1cc(-c2ccc3c(c2)c2ccccc2n3-c2ccccc2)c2c(c1)c1cc(C(C)(C)C)cc3c4c5c6cc7ccccc7c7c8c9ccccc9cc(-c9ccc%10c(c9)c9ccccc9n%10-c9ccccc9)c8n(c5cnc4n2c13)c67]

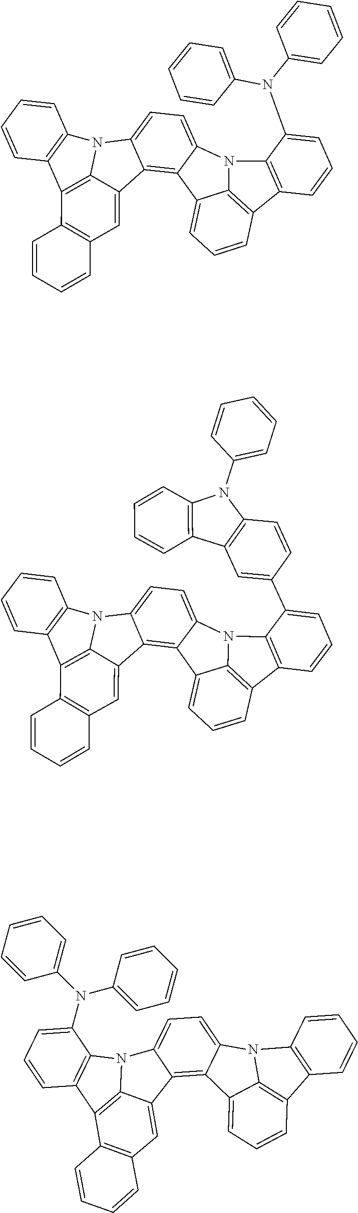 [SMILES: c1ccc(N(c2ccccc2)c2cccc3c4cccc5c6c7c8cc9ccccc9c9c%10ccccc%10n(c7ccc6n(c23)c45)c89)cc1 ; c1ccc(-n2c3ccccc3c3cc(-c4cccc5c6cccc7c8c9c%10cc%11ccccc%11c%11c%12ccccc%12n(c9ccc8n(c45)c67)c%10%11)ccc32)cc1 ; c1ccc(N(c2ccccc2)c2cccc3c4c5ccccc5cc5c6c7c8cccc9c%10ccccc%10n(c7ccc6n(c23)c54)c98)cc1]

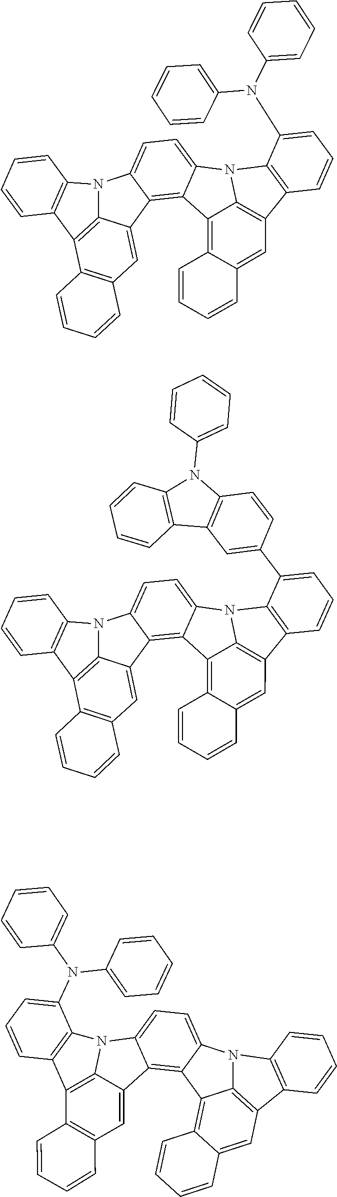 [SMILES: c1ccc(N(c2ccccc2)c2cccc3c4cc5ccccc5c5c6c7c8cc9ccccc9c9c%10ccccc%10n(c7ccc6n(c23)c45)c89)cc1 ; c1ccc(-n2c3ccccc3c3cc(-c4cccc5c6cc7ccccc7c7c8c9c%10cc%11ccccc%11c%11c%12ccccc%12n(c9ccc8n(c45)c67)c%10%11)ccc32)cc1 ; c1ccc(N(c2ccccc2)c2cccc3c4c5ccccc5cc5c6c7c8c9ccccc9cc9c%10ccccc%10n(c7ccc6n(c23)c54)c98)cc1]

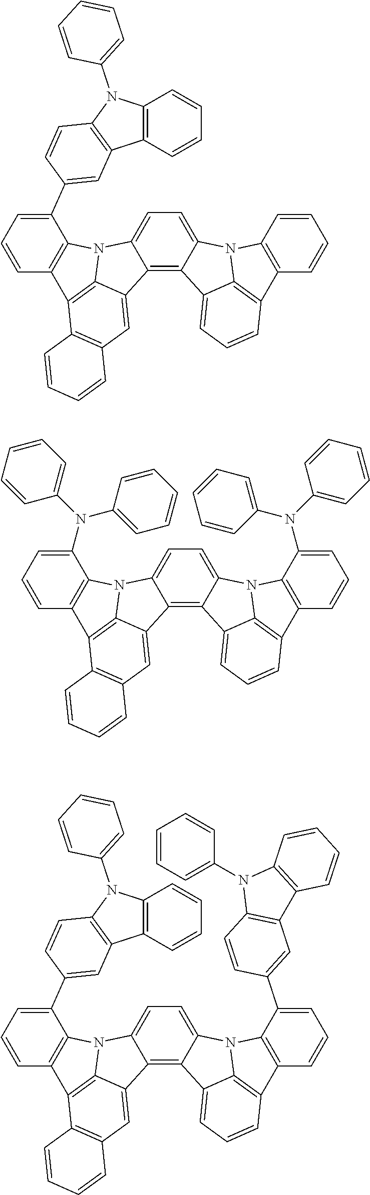 [SMILES: c1ccc(-n2c3ccccc3c3cc(-c4cccc5c6c7ccccc7cc7c8c9c%10cccc%11c%12ccccc%12n(c9ccc8n(c45)c76)c%11%10)ccc32)cc1 ; c1ccc(N(c2ccccc2)c2cccc3c4cccc5c6c7c8cc9ccccc9c9c%10cccc(N(c%11ccccc%11)c%11ccccc%11)c%10n(c7ccc6n(c23)c45)c89)cc1 ; c1ccc(-n2c3ccccc3c3cc(-c4cccc5c6cccc7c8c9c%10cc%11ccccc%11c%11c%12cccc(-c%13ccc%14c(c%13)c%13ccccc%13n%14-c%13ccccc%13)c%12n(c9ccc8n(c45)c67)c%10%11)ccc32)cc1]

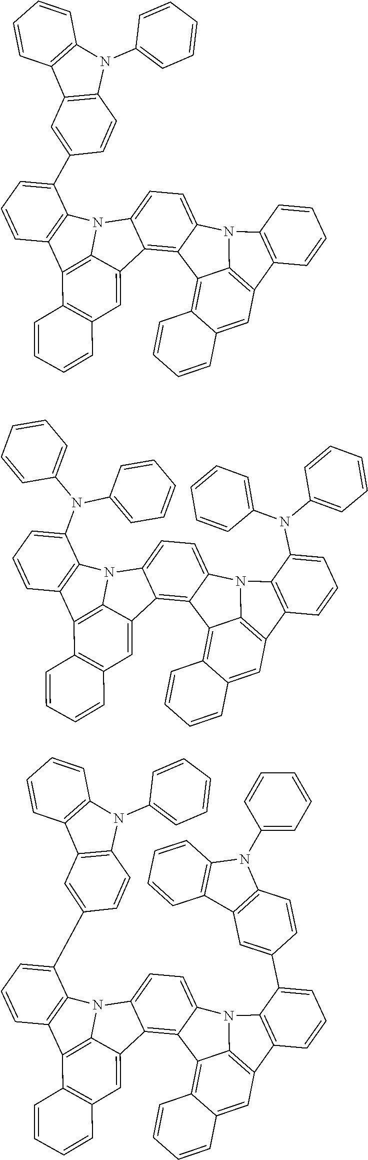

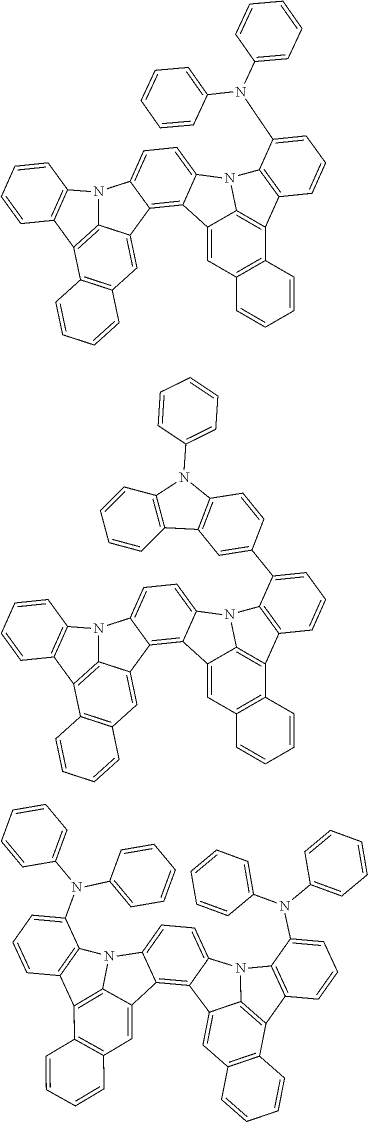

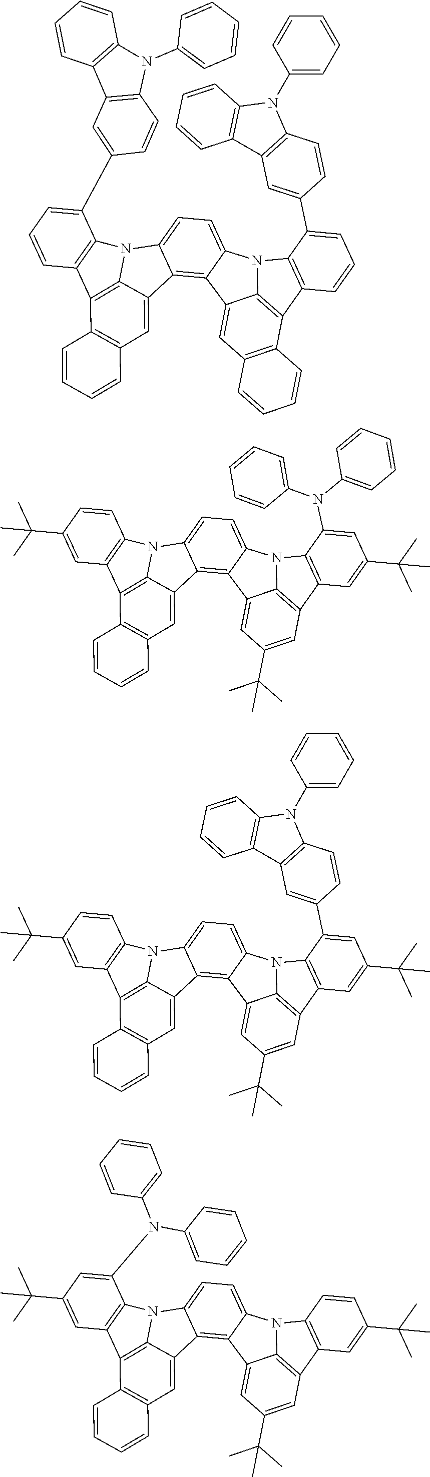

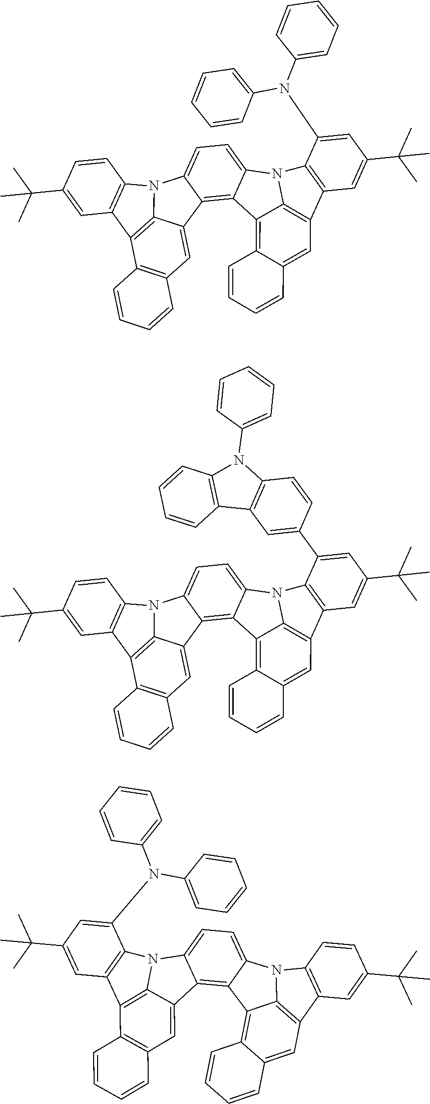

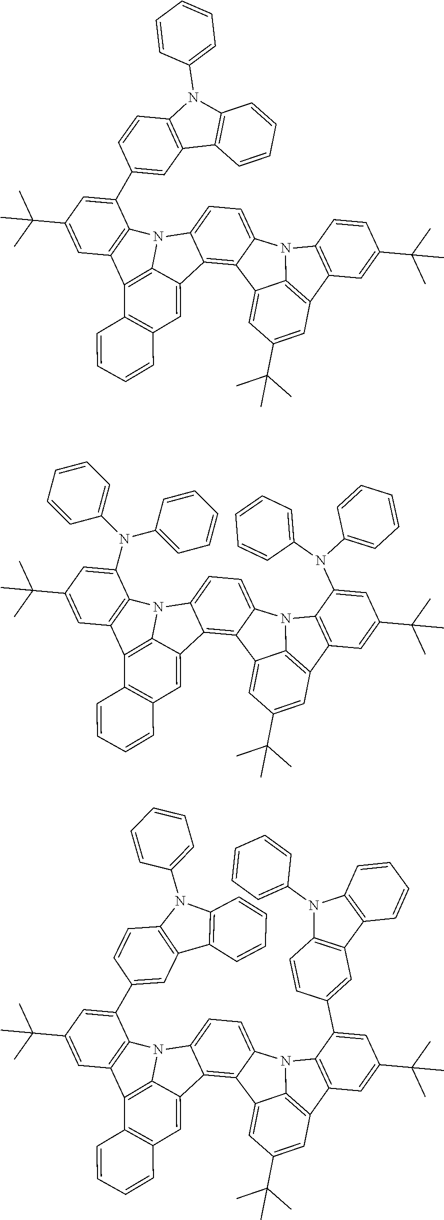

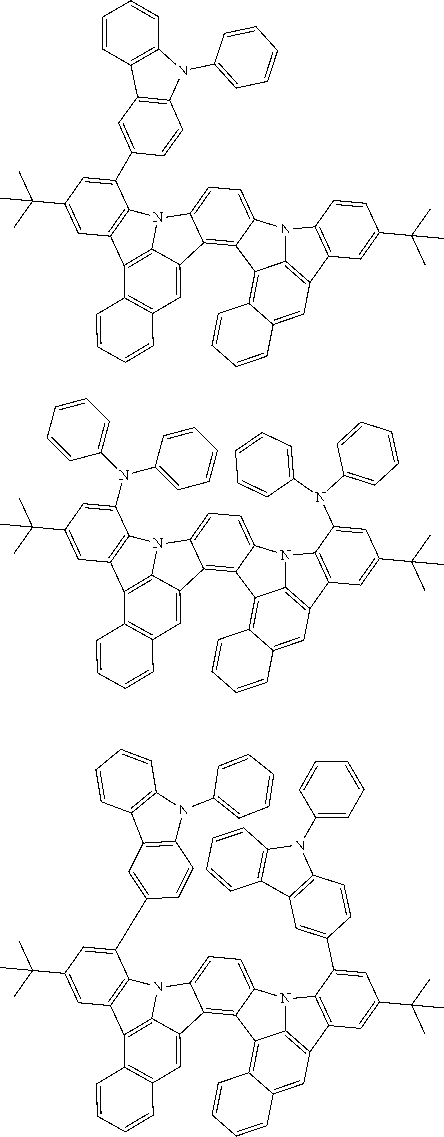

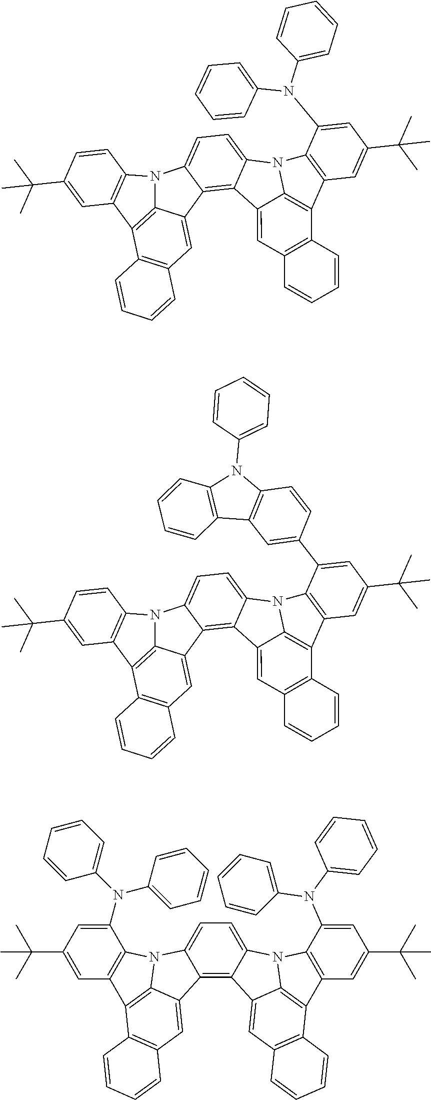

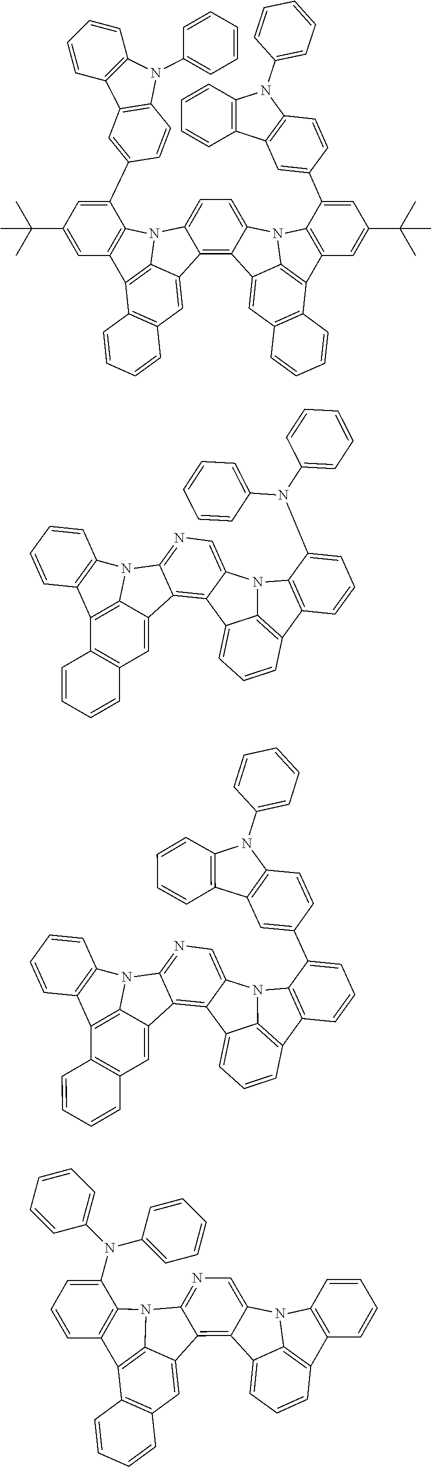

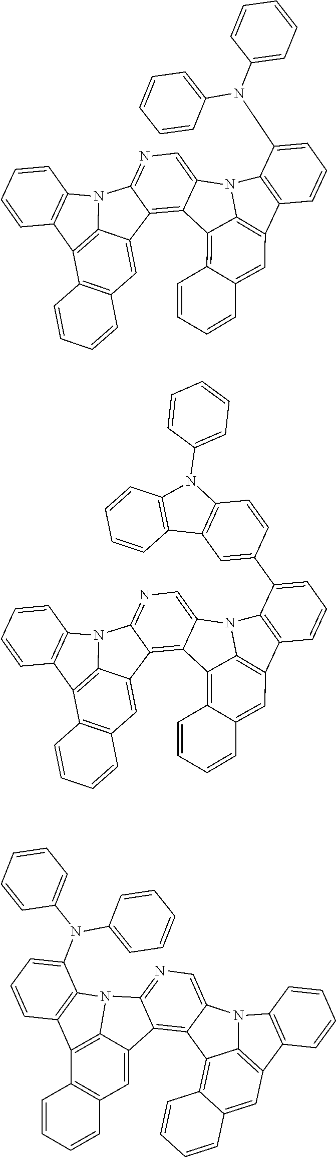 [SMILES: c1ccc(N(c2ccccc2)c2cccc3c4cc5ccccc5c5c6c7c8cc9ccccc9c9c%10ccccc%10n(c7ncc6n(c23)c45)c89)cc1 ; c1ccc(-n2c3ccccc3c3cc(-c4cccc5c6cc7ccccc7c7c8c9c%10cc%11ccccc%11c%11c%12ccccc%12n(c9ncc8n(c45)c67)c%10%11)ccc32)cc1 ; c1ccc(N(c2ccccc2)c2cccc3c4c5ccccc5cc5c6c7c8c9ccccc9cc9c%10ccccc%10n(c7cnc6n(c23)c54)c98)cc1]

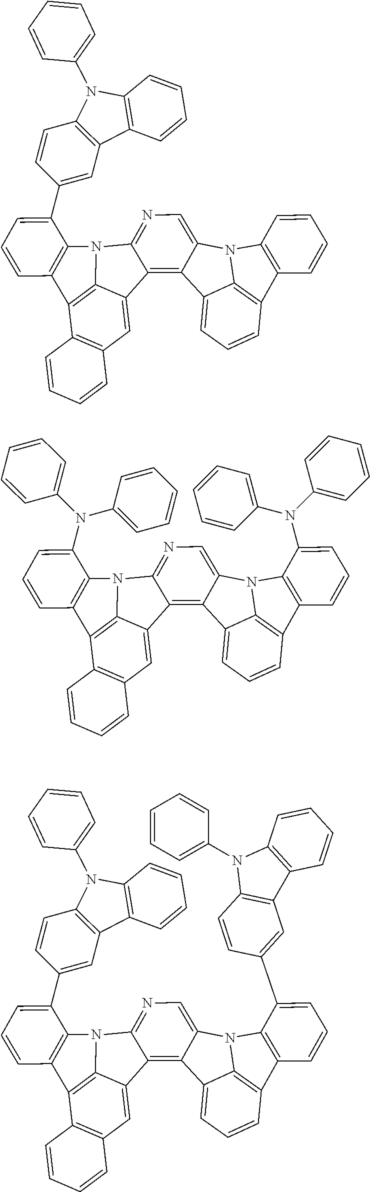 [SMILES: c1ccc(-n2c3ccccc3c3cc(-c4cccc5c6c7ccccc7cc7c8c9c%10cccc%11c%12ccccc%12n(c9cnc8n(c45)c76)c%11%10)ccc32)cc1 ; c1ccc(N(c2ccccc2)c2cccc3c4cccc5c6c7c8cc9ccccc9c9c%10cccc(N(c%11ccccc%11)c%11ccccc%11)c%10n(c7ncc6n(c23)c45)c89)cc1 ; c1ccc(-n2c3ccccc3c3cc(-c4cccc5c6cccc7c8c9c%10cc%11ccccc%11c%11c%12cccc(-c%13ccc%14c(c%13)c%13ccccc%13n%14-c%13ccccc%13)c%12n(c9ncc8n(c45)c67)c%10%11)ccc32)cc1]

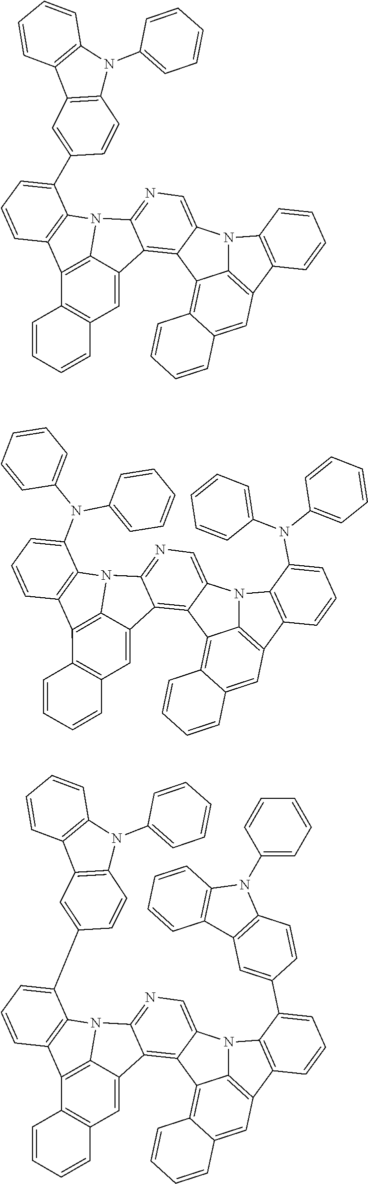 [SMILES: c1ccc(-n2c3ccccc3c3cc(-c4cccc5c6c7ccccc7cc7c8c9c%10c%11ccccc%11cc%11c%12ccccc%12n(c9cnc8n(c45)c76)c%11%10)ccc32)cc1 ; c1ccc(N(c2ccccc2)c2cccc3c4cc5ccccc5c5c6c7c8cc9ccccc9c9c%10cccc(N(c%11ccccc%11)c%11ccccc%11)c%10n(c7ncc6n(c23)c45)c89)cc1 ; c1ccc(-n2c3ccccc3c3cc(-c4cccc5c6cc7ccccc7c7c8c9c%10cc%11ccccc%11c%11c%12cccc(-c%13ccc%14c(c%13)c%13ccccc%13n%14-c%13ccccc%13)c%12n(c9ncc8n(c45)c67)c%10%11)ccc32)cc1]

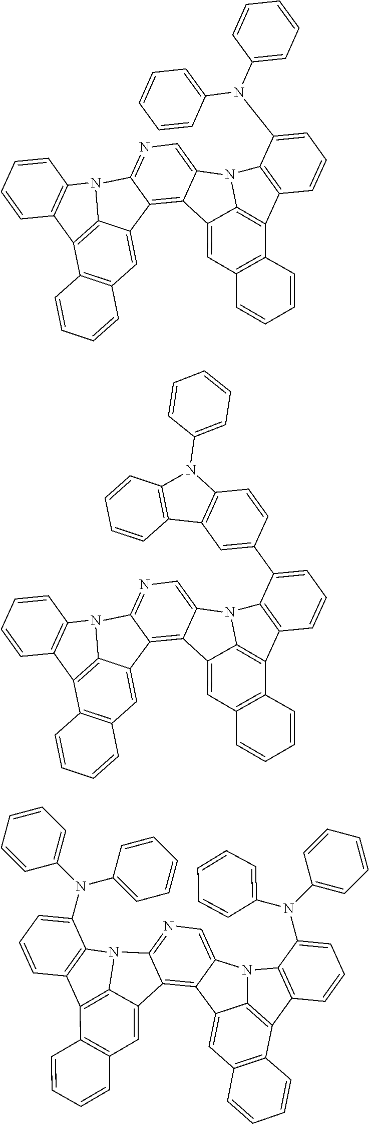

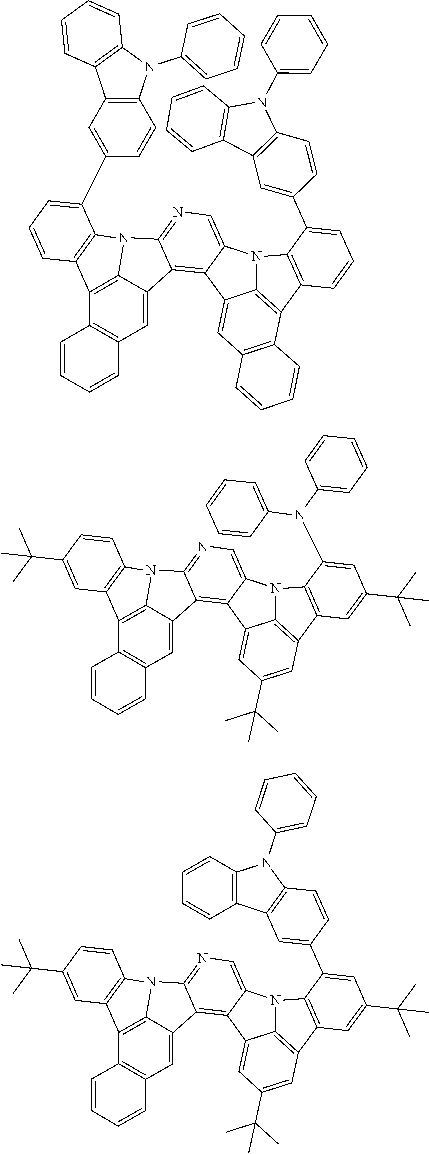

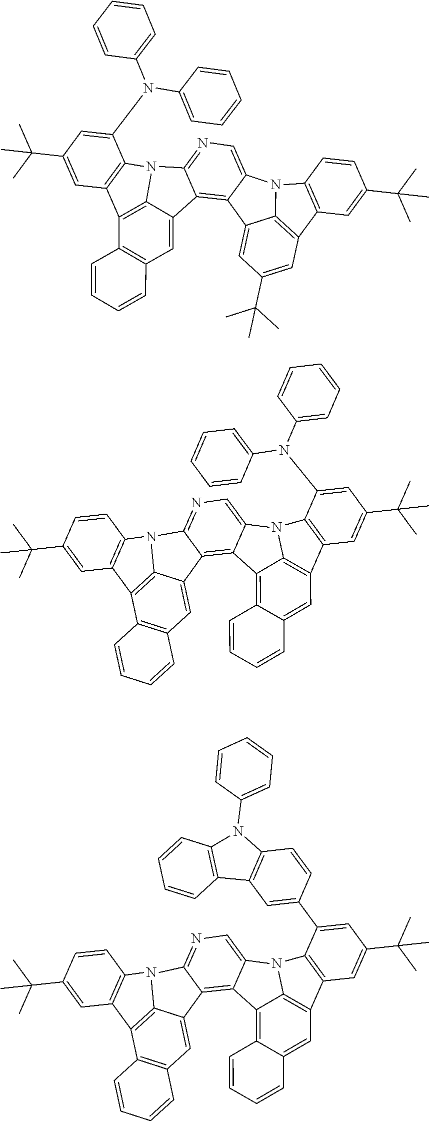

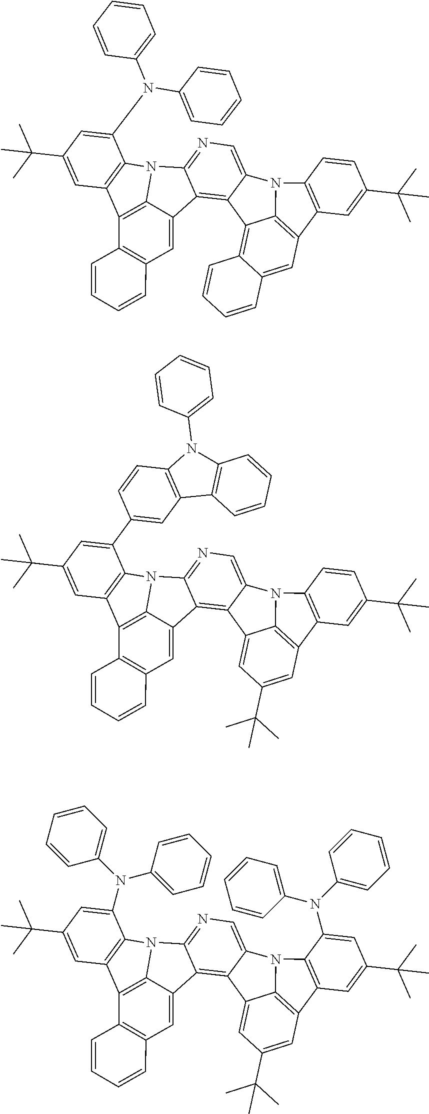

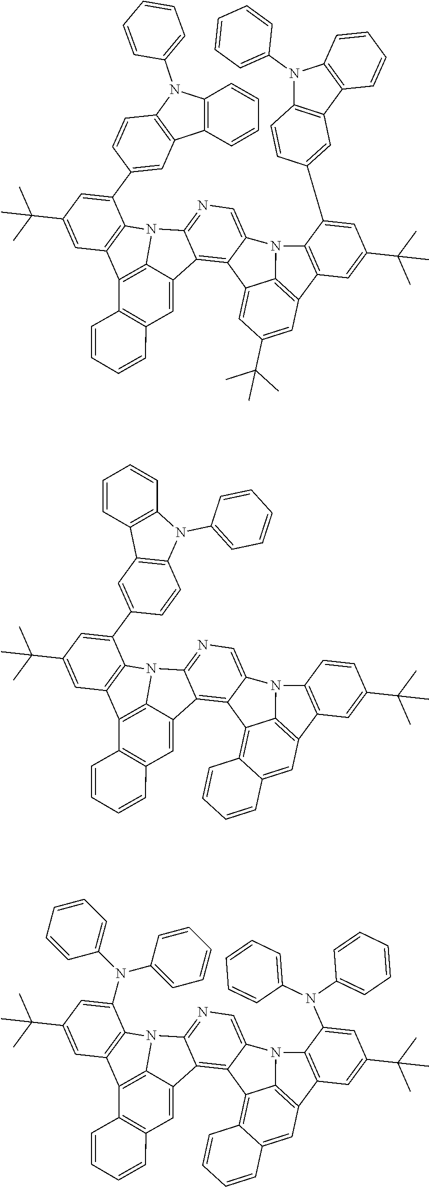

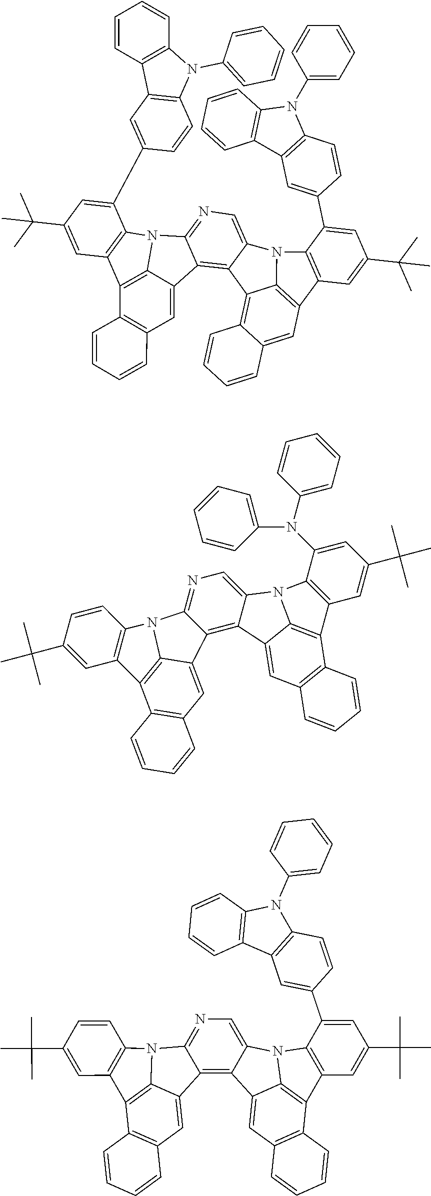

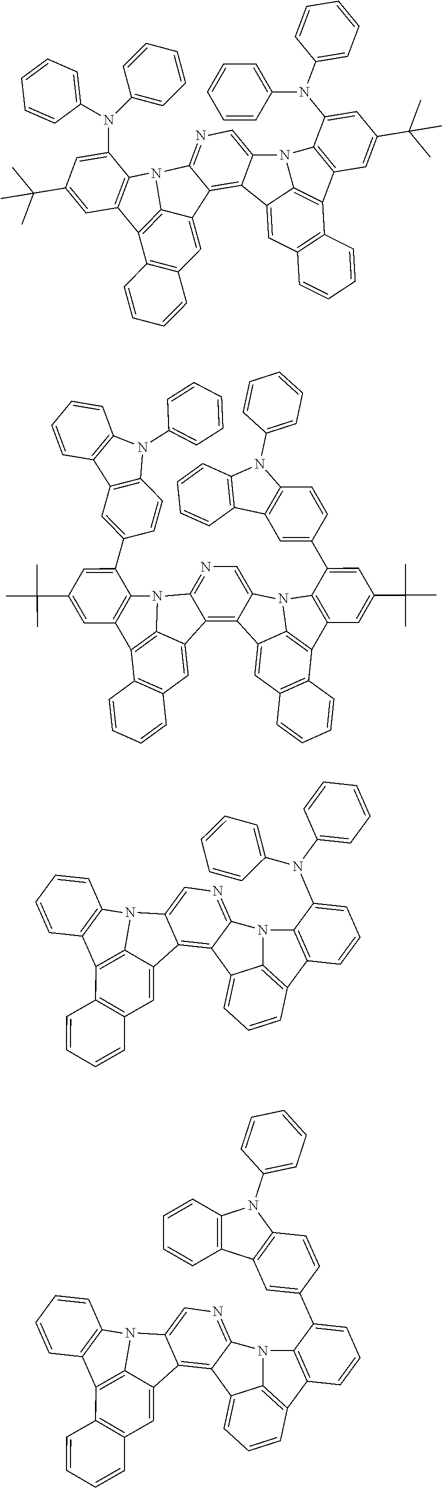

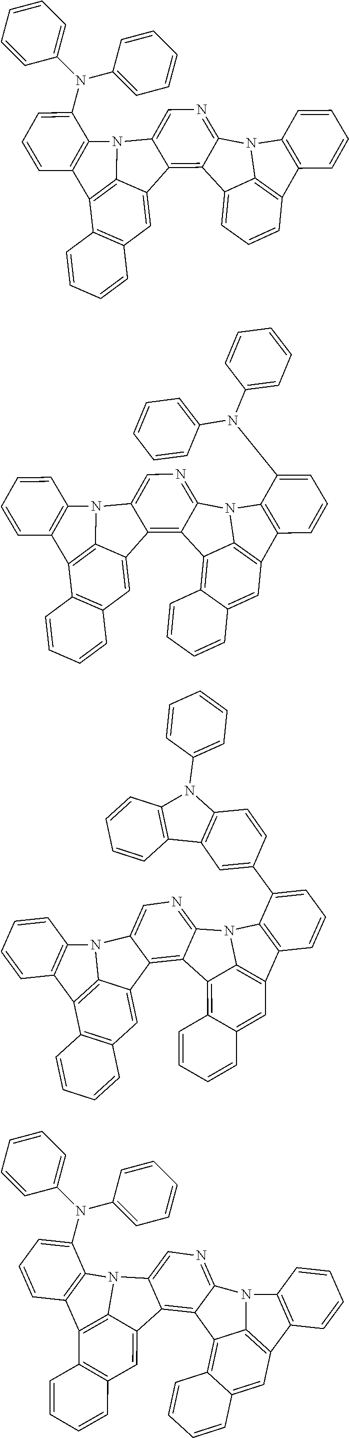

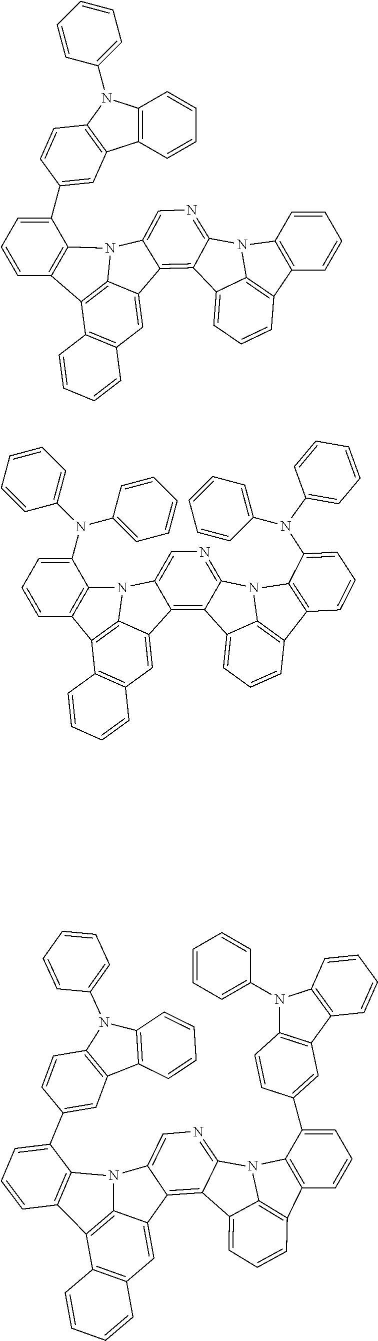 [SMILES: c1ccc(-n2c3ccccc3c3cc(-c4cccc5c6c7ccccc7cc7c8c9c%10cccc%11c%12ccccc%12n(c9ncc8n(c45)c76)c%11%10)ccc32)cc1 ; c1ccc(N(c2ccccc2)c2cccc3c4c5ccccc5cc5c6c7c8cccc9c%10cccc(N(c%11ccccc%11)c%11ccccc%11)c%10n(c7ncc6n(c23)c54)c98)cc1 ; c1ccc(-n2c3ccccc3c3cc(-c4cccc5c6c7ccccc7cc7c8c9c%10cccc%11c%12cccc(-c%13ccc%14c(c%13)c%13ccccc%13n%14-c%13ccccc%13)c%12n(c9ncc8n(c45)c76)c%11%10)ccc32)cc1]

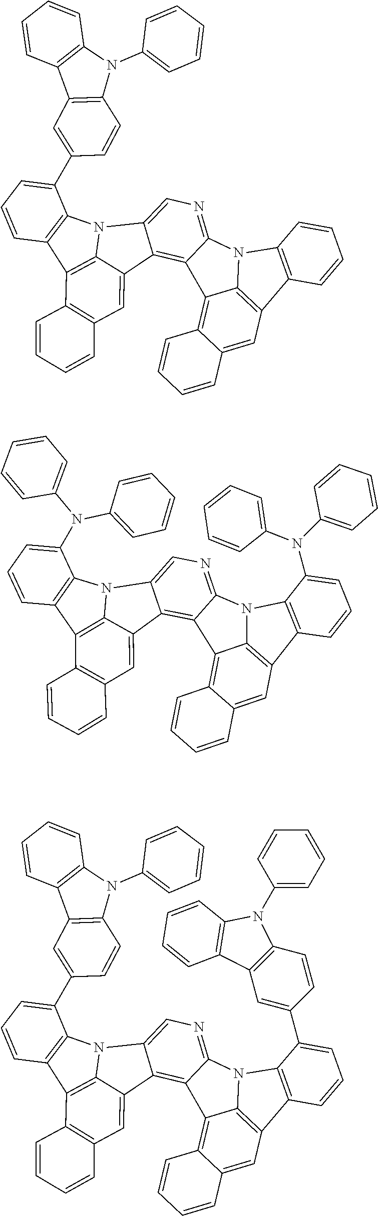 [SMILES: c1ccc(-n2c3ccccc3c3cc(-c4cccc5c6c7ccccc7cc7c8c9c%10c%11ccccc%11cc%11c%12ccccc%12n(c9ncc8n(c45)c76)c%11%10)ccc32)cc1 ; c1ccc(N(c2ccccc2)c2cccc3c4c5ccccc5cc5c6c7c8c9ccccc9cc9c%10cccc(N(c%11ccccc%11)c%11ccccc%11)c%10n(c7ncc6n(c23)c54)c98)cc1 ; c1ccc(-n2c3ccccc3c3cc(-c4cccc5c6c7ccccc7cc7c8c9c%10c%11ccccc%11cc%11c%12cccc(-c%13ccc%14c(c%13)c%13ccccc%13n%14-c%13ccccc%13)c%12n(c9ncc8n(c45)c76)c%11%10)ccc32)cc1]

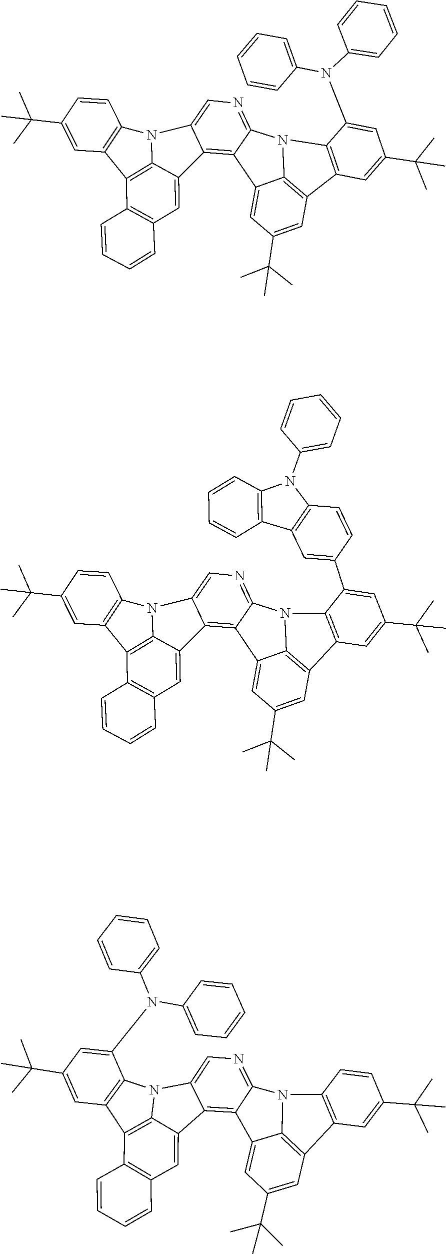

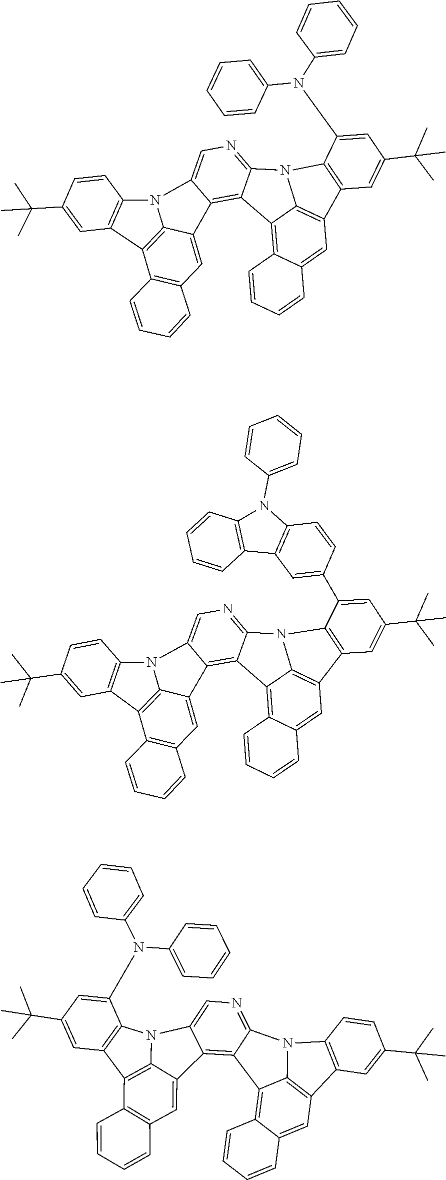

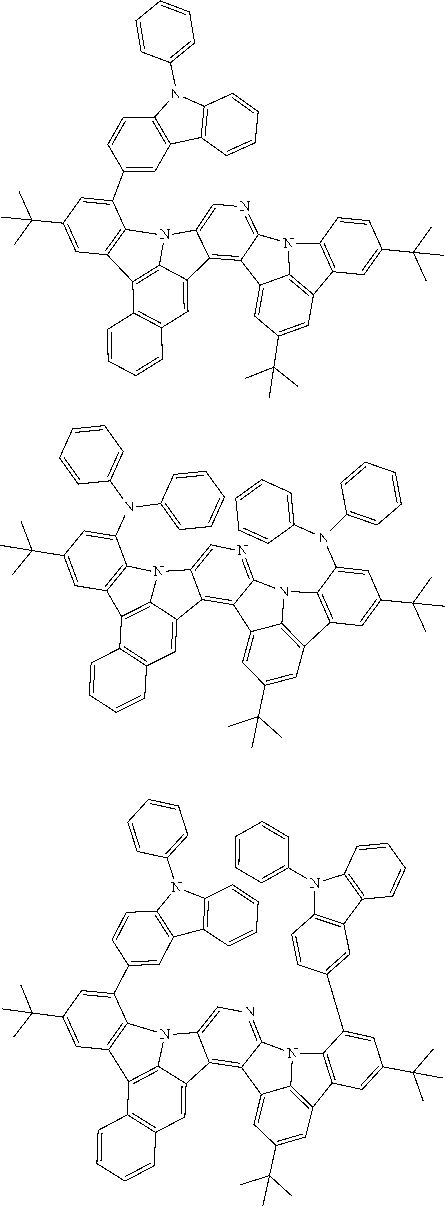

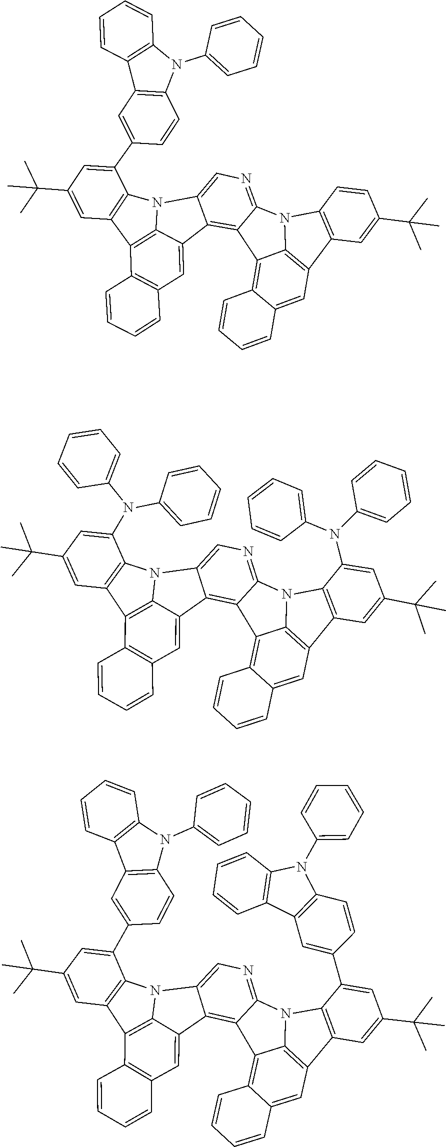

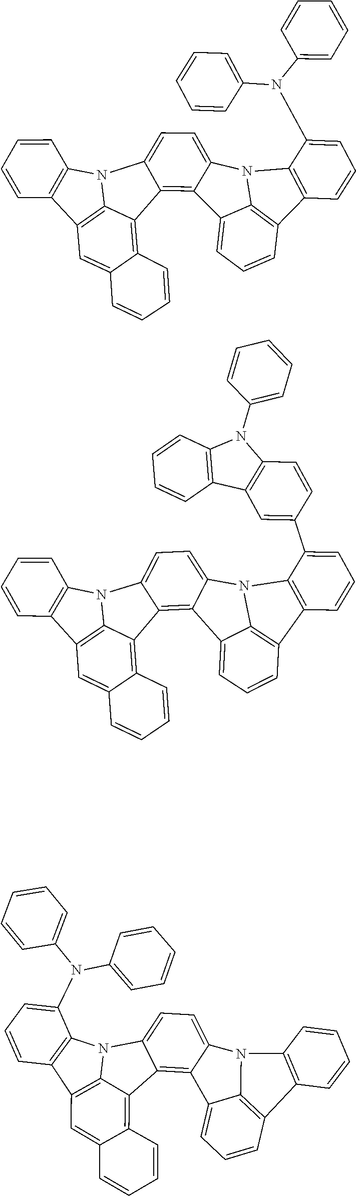 [SMILES: c1ccc(N(c2ccccc2)c2cccc3c4cccc5c6c7c8c9ccccc9cc9c%10ccccc%10n(c7ccc6n(c23)c45)c98)cc1 ; c1ccc(-n2c3ccccc3c3cc(-c4cccc5c6cccc7c8c9c%10c%11ccccc%11cc%11c%12ccccc%12n(c9ccc8n(c45)c67)c%11%10)ccc32)cc1 ; c1ccc(N(c2ccccc2)c2cccc3c4cc5ccccc5c5c6c7c8cccc9c%10ccccc%10n(c7ccc6n(c23)c45)c98)cc1]

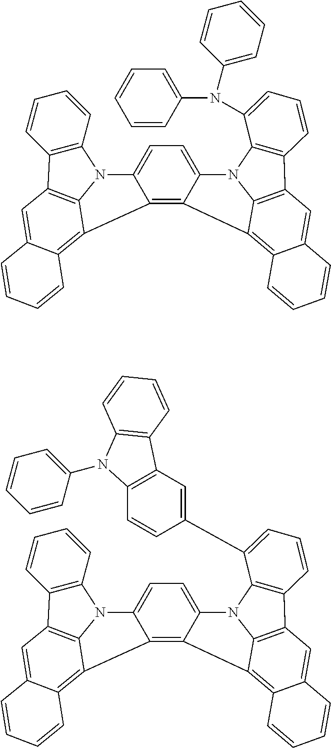 [SMILES: c1ccc(N(c2ccccc2)c2cccc3c4cc5ccccc5c5c6c7c8c9ccccc9cc9c%10ccccc%10n(c7ccc6n(c23)c45)c98)cc1 ; c1ccc(-n2c3ccccc3c3cc(-c4cccc5c6cc7ccccc7c7c8c9c%10c%11ccccc%11cc%11c%12ccccc%12n(c9ccc8n(c45)c67)c%11%10)ccc32)cc1]

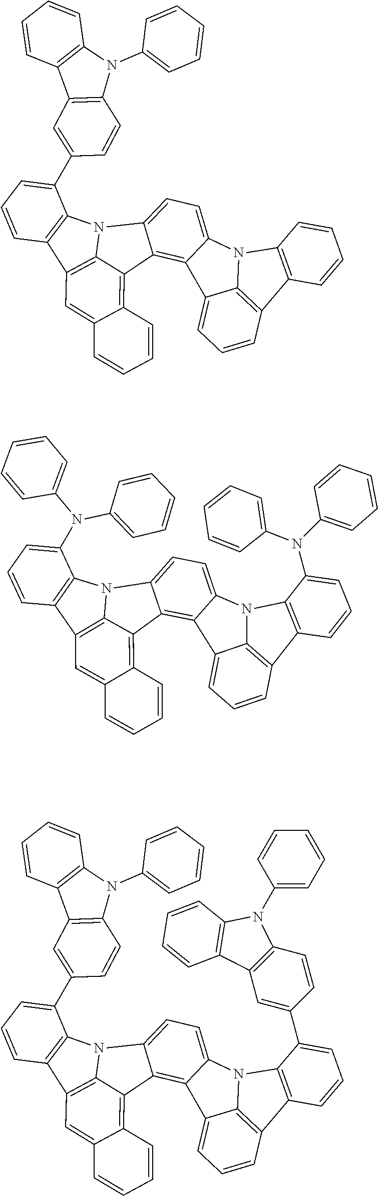 [SMILES: c1ccc(-n2c3ccccc3c3cc(-c4cccc5c6cc7ccccc7c7c8c9c%10cccc%11c%12ccccc%12n(c9ccc8n(c45)c67)c%11%10)ccc32)cc1 ; c1ccc(N(c2ccccc2)c2cccc3c4cccc5c6c7c8c9ccccc9cc9c%10cccc(N(c%11ccccc%11)c%11ccccc%11)c%10n(c7ccc6n(c23)c45)c98)cc1 ; c1ccc(-n2c3ccccc3c3cc(-c4cccc5c6cccc7c8c9c%10c%11ccccc%11cc%11c%12cccc(-c%13ccc%14c(c%13)c%13ccccc%13n%14-c%13ccccc%13)c%12n(c9ccc8n(c45)c67)c%11%10)ccc32)cc1]

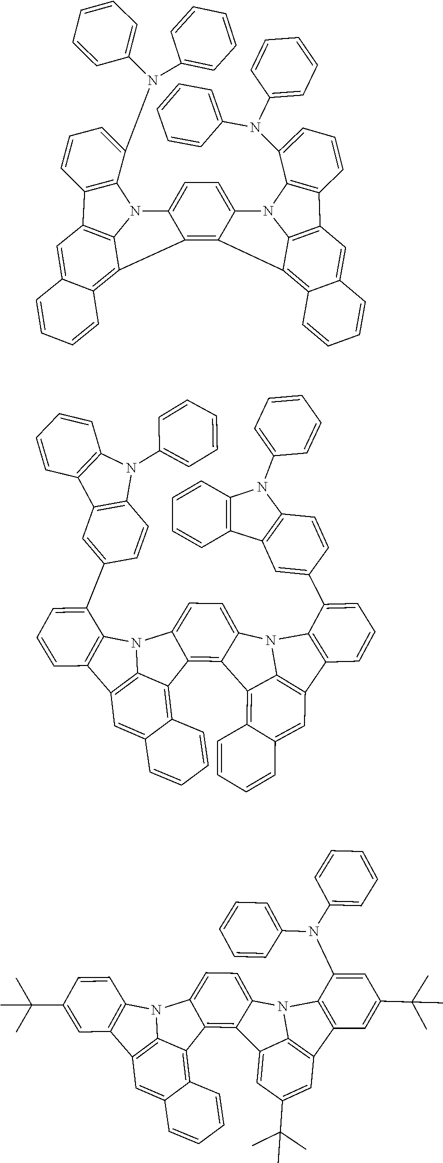

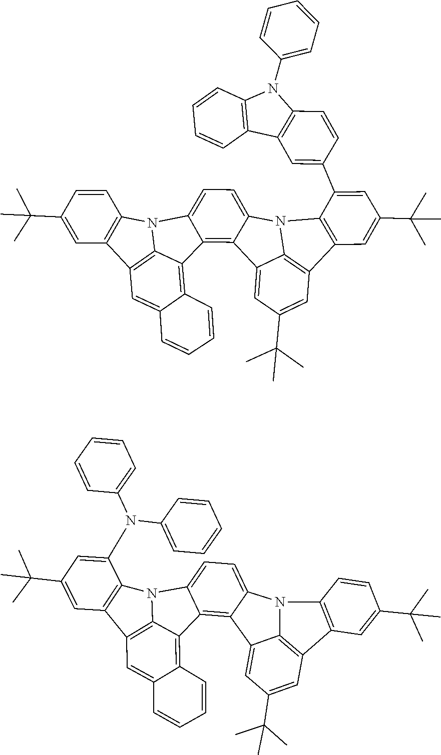

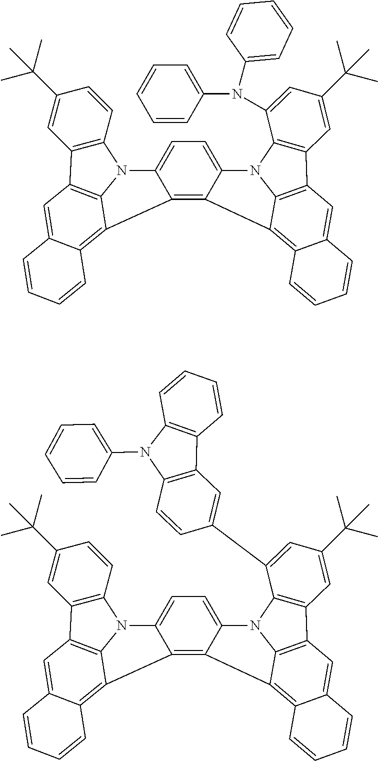

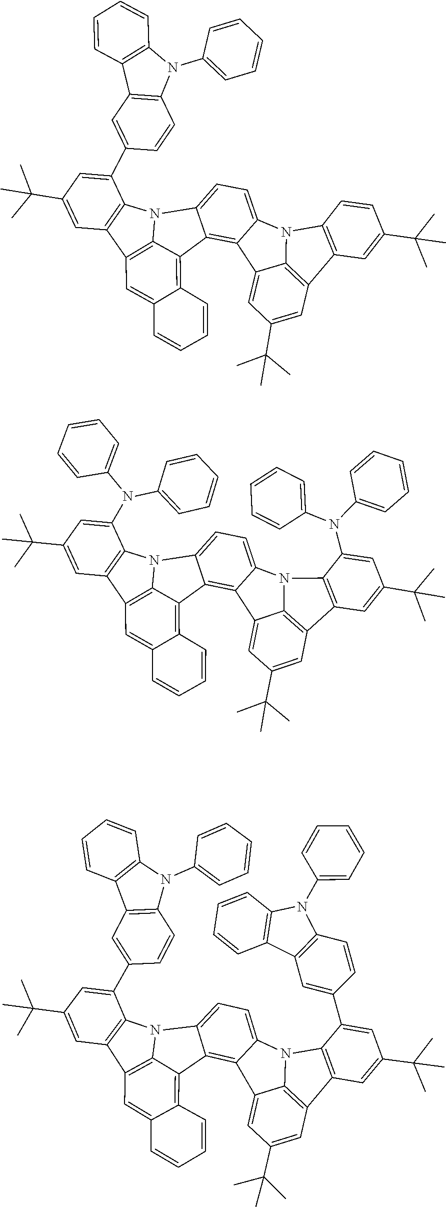

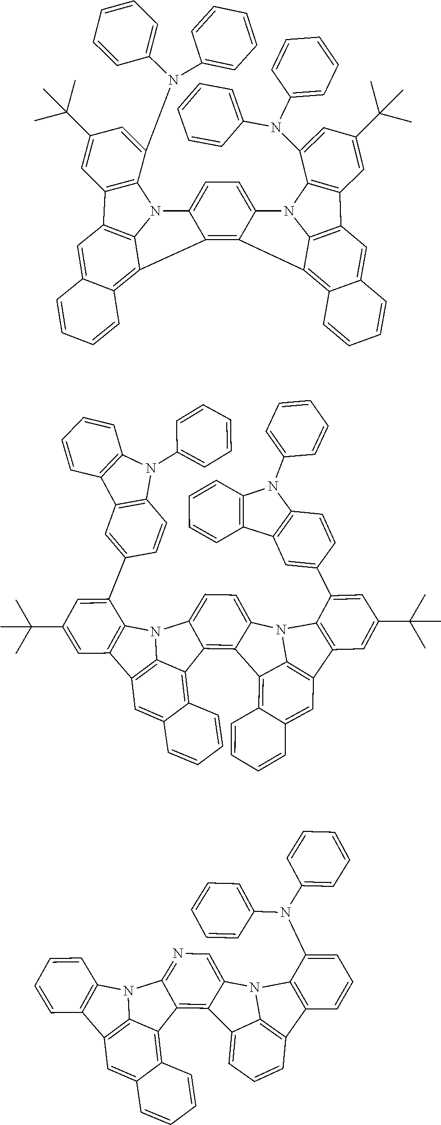

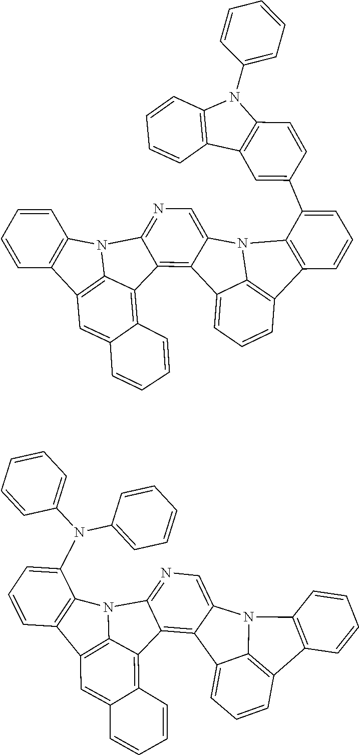 [SMILES: c1ccc(-n2c3ccccc3c3cc(-c4cccc5c6cccc7c8c9c%10c%11ccccc%11cc%11c%12ccccc%12n(c9ncc8n(c45)c67)c%11%10)ccc32)cc1 ; c1ccc(N(c2ccccc2)c2cccc3c4cc5ccccc5c5c6c7c8cccc9c%10ccccc%10n(c7cnc6n(c23)c45)c98)cc1]

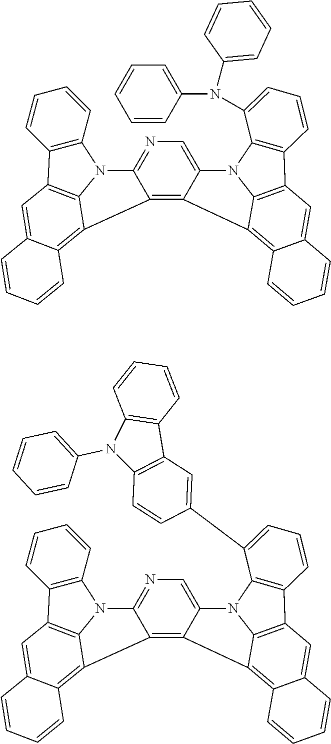 [SMILES: c1ccc(N(c2ccccc2)c2cccc3c4cc5ccccc5c5c6c7c8c9ccccc9cc9c%10ccccc%10n(c7ncc6n(c23)c45)c98)cc1 ; c1ccc(-n2c3ccccc3c3cc(-c4cccc5c6cc7ccccc7c7c8c9c%10c%11ccccc%11cc%11c%12ccccc%12n(c9ncc8n(c45)c67)c%11%10)ccc32)cc1]

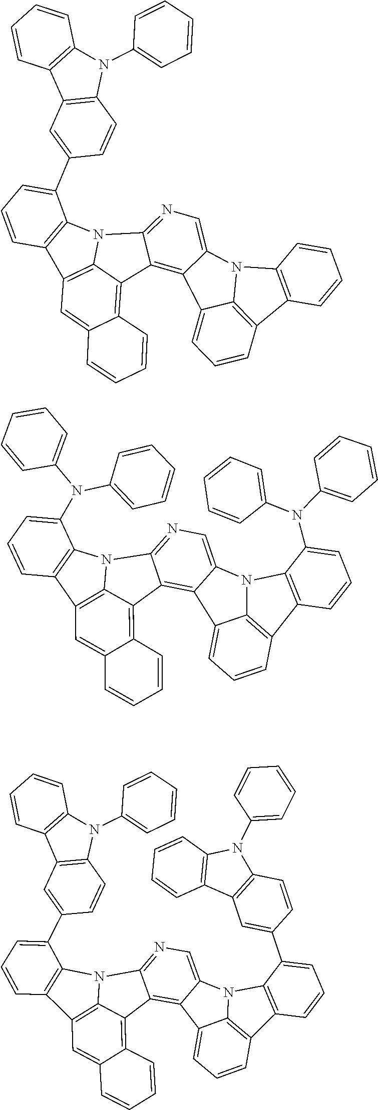 [SMILES: c1ccc(-n2c3ccccc3c3cc(-c4cccc5c6cc7ccccc7c7c8c9c%10cccc%11c%12ccccc%12n(c9cnc8n(c45)c67)c%11%10)ccc32)cc1 ; c1ccc(N(c2ccccc2)c2cccc3c4cccc5c6c7c8c9ccccc9cc9c%10cccc(N(c%11ccccc%11)c%11ccccc%11)c%10n(c7ncc6n(c23)c45)c98)cc1 ; c1ccc(-n2c3ccccc3c3cc(-c4cccc5c6cccc7c8c9c%10c%11ccccc%11cc%11c%12cccc(-c%13ccc%14c(c%13)c%13ccccc%13n%14-c%13ccccc%13)c%12n(c9ncc8n(c45)c67)c%11%10)ccc32)cc1]

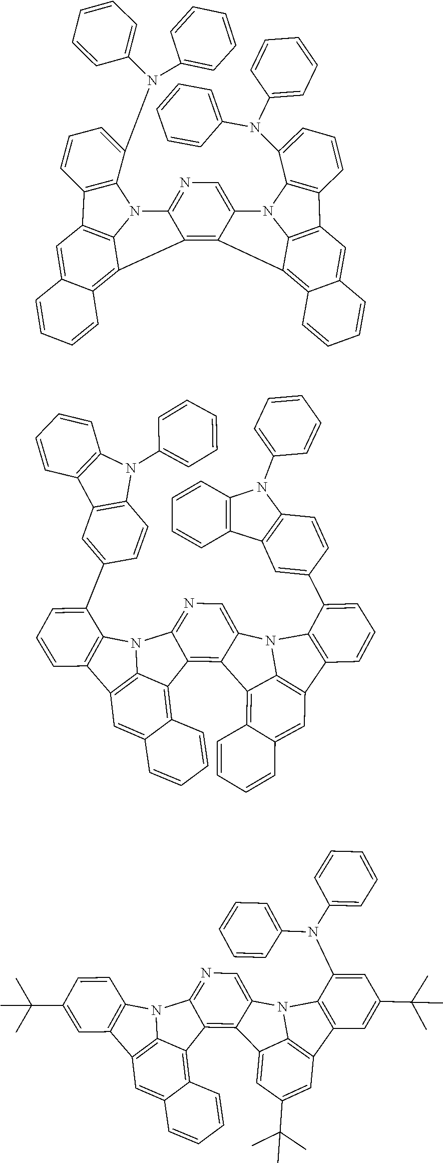

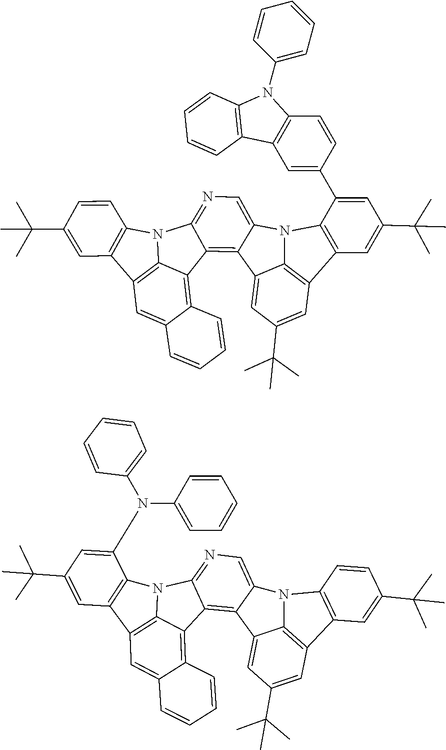

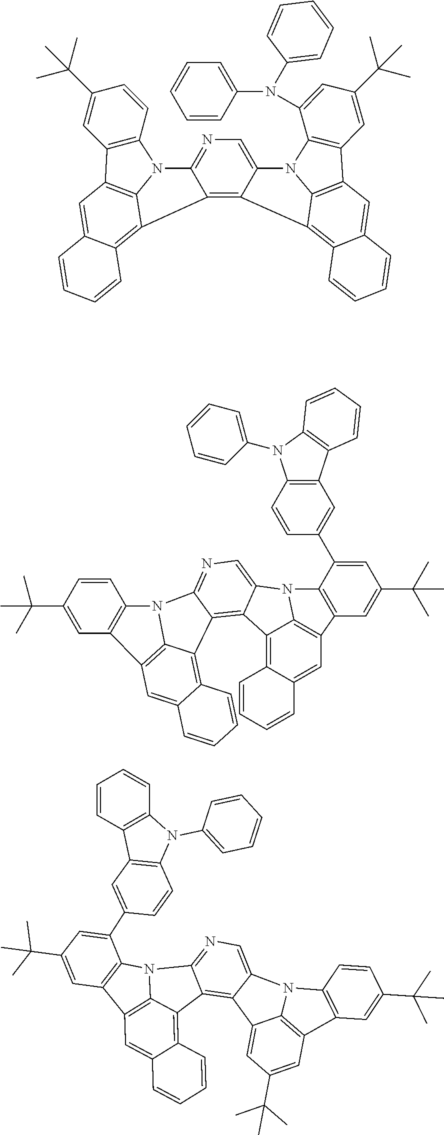

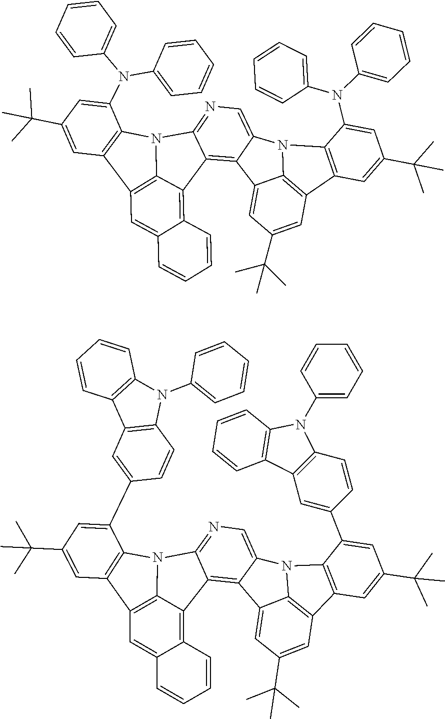

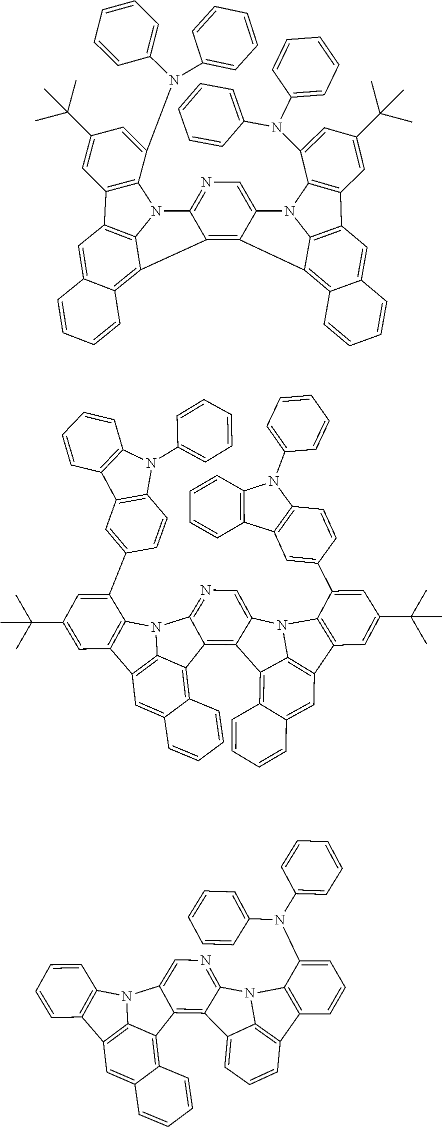

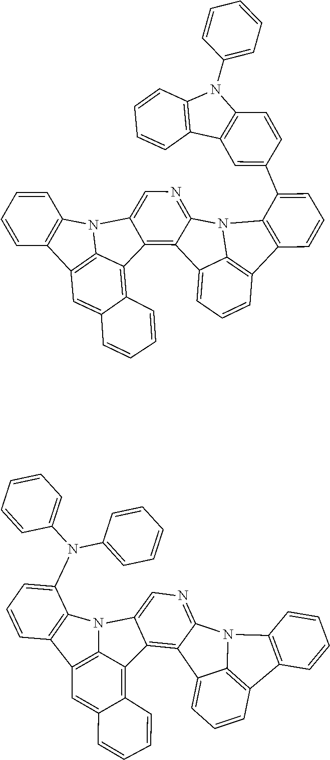 [SMILES: c1ccc(-n2c3ccccc3c3cc(-c4cccc5c6cccc7c8c9c%10c%11ccccc%11cc%11c%12ccccc%12n(c9cnc8n(c45)c67)c%11%10)ccc32)cc1 ; c1ccc(N(c2ccccc2)c2cccc3c4cc5ccccc5c5c6c7c8cccc9c%10ccccc%10n(c7ncc6n(c23)c45)c98)cc1]

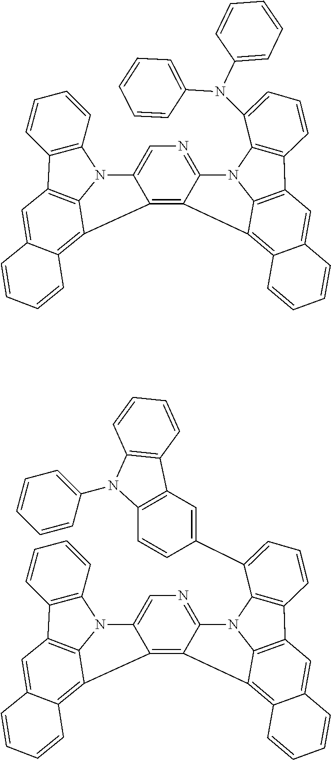

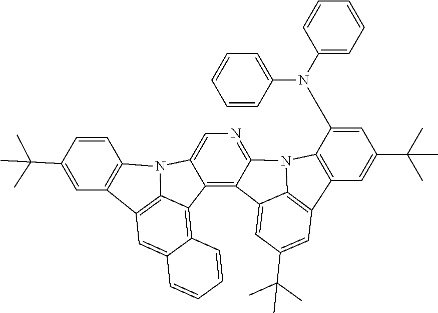 [SMILES: CC(C)(C)c1ccc2c(c1)c1cc3ccccc3c3c4c5c6cc(C(C)(C)C)cc7c8cc(C(C)(C)C)cc(N(c9ccccc9)c9ccccc9)c8n(c5ncc4n2c13)c76]

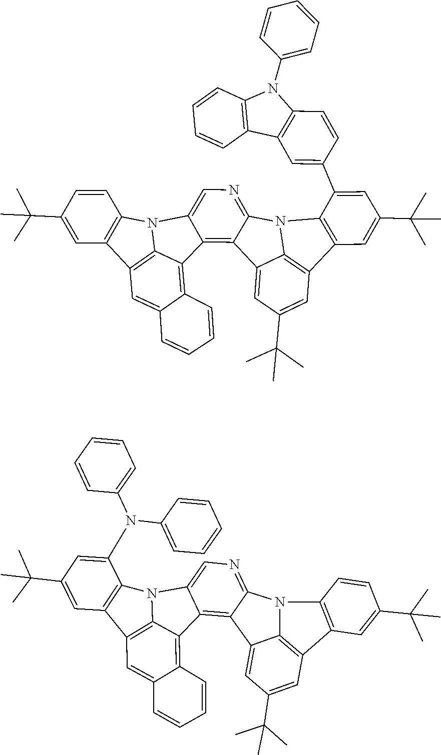

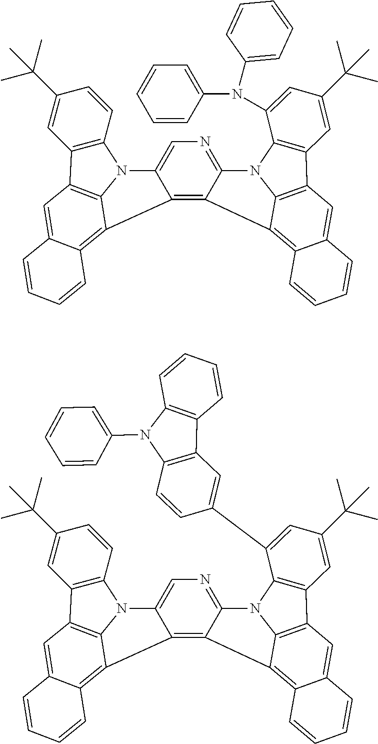

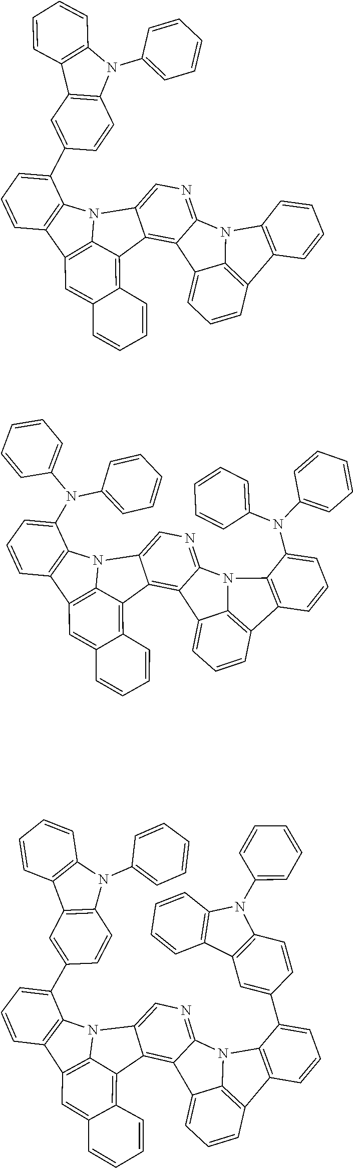 [SMILES: c1ccc(-n2c3ccccc3c3cc(-c4cccc5c6cc7ccccc7c7c8c9c%10cccc%11c%12ccccc%12n(c9ncc8n(c45)c67)c%11%10)ccc32)cc1 ; c1ccc(N(c2ccccc2)c2cccc3c4cc5ccccc5c5c6c7c8cccc9c%10cccc(N(c%11ccccc%11)c%11ccccc%11)c%10n(c7ncc6n(c23)c45)c98)cc1 ; c1ccc(-n2c3ccccc3c3cc(-c4cccc5c6cc7ccccc7c7c8c9c%10cccc%11c%12cccc(-c%13ccc%14c(c%13)c%13ccccc%13n%14-c%13ccccc%13)c%12n(c9ncc8n(c45)c67)c%11%10)ccc32)cc1]

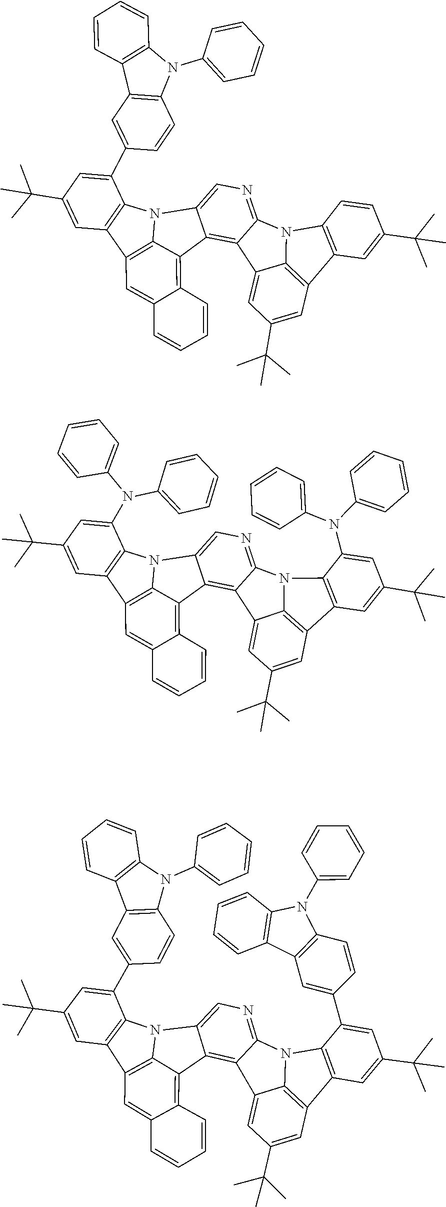

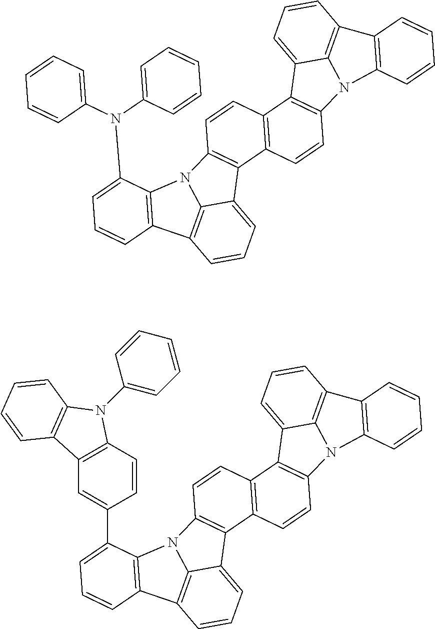 [SMILES: c1ccc(N(c2ccccc2)c2cccc3c4cccc5c6c7ccc8c(c7ccc6n(c23)c45)c2cccc3c4ccccc4n8c32)cc1 ; c1ccc(-n2c3ccccc3c3cc(-c4cccc5c6cccc7c8c9ccc%10c(c9ccc8n(c45)c67)c4cccc5c6ccccc6n%10c54)ccc32)cc1]

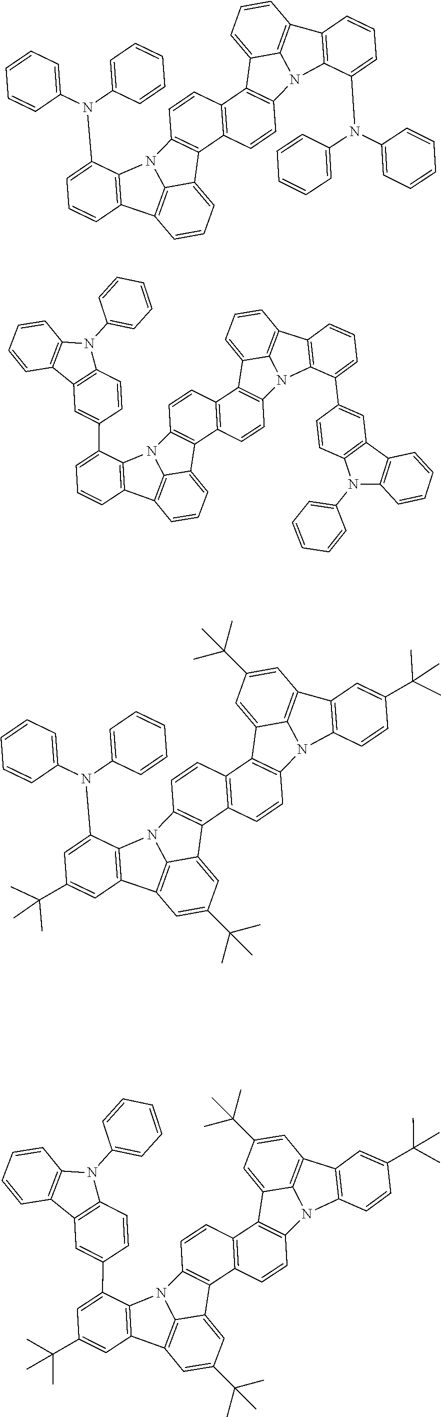 [SMILES: c1ccc(N(c2ccccc2)c2cccc3c4cccc5c6c7ccc8c(c7ccc6n(c23)c45)c2cccc3c4cccc(N(c5ccccc5)c5ccccc5)c4n8c32)cc1 ; c1ccc(-n2c3ccccc3c3cc(-c4cccc5c6cccc7c8c9ccc%10c(c9ccc8n(c45)c67)c4cccc5c6cccc(-c7ccc8c(c7)c7ccccc7n8-c7ccccc7)c6n%10c54)ccc32)cc1 ; CC(C)(C)c1ccc2c(c1)c1cc(C(C)(C)C)cc3c4c5ccc6c(c5ccc4n2c13)c1cc(C(C)(C)C)cc2c3cc(C(C)(C)C)cc(N(c4ccccc4)c4ccccc4)c3n6c21 ; CC(C)(C)c1ccc2c(c1)c1cc(C(C)(C)C)cc3c4c5ccc6c(c5ccc4n2c13)c1cc(C(C)(C)C)cc2c3cc(C(C)(C)C)cc(-c4ccc5c(c4)c4ccccc4n5-c4ccccc4)c3n6c21]

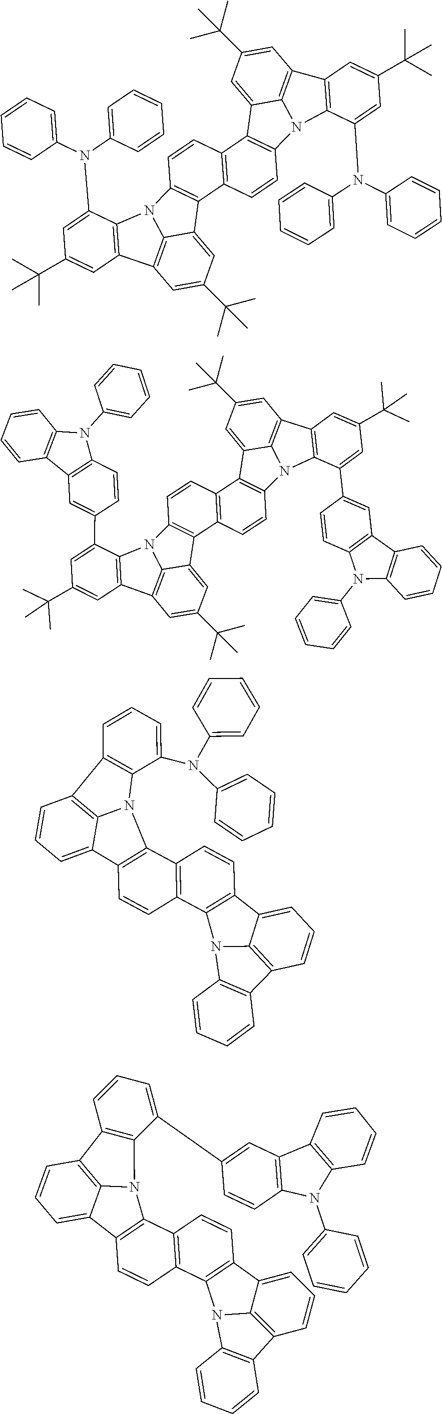

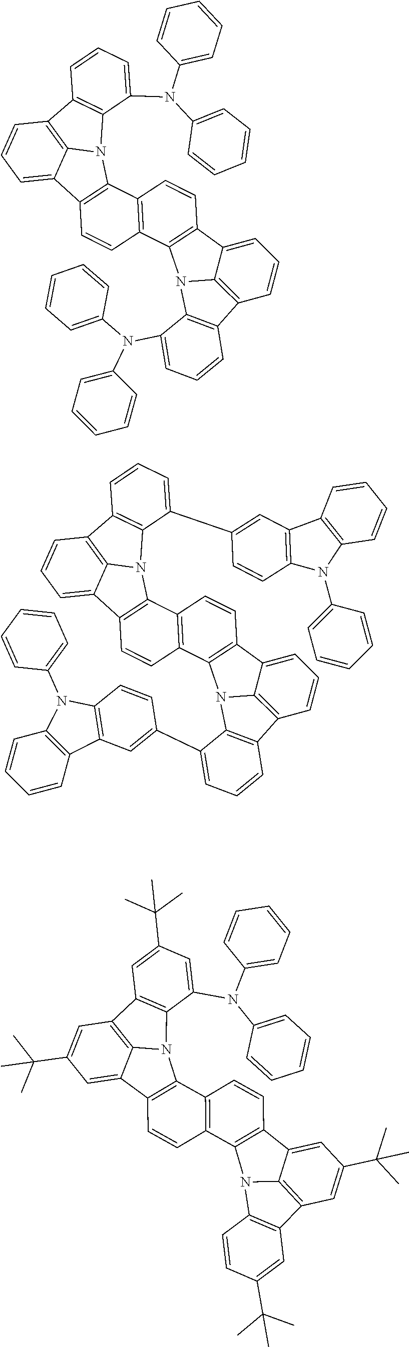

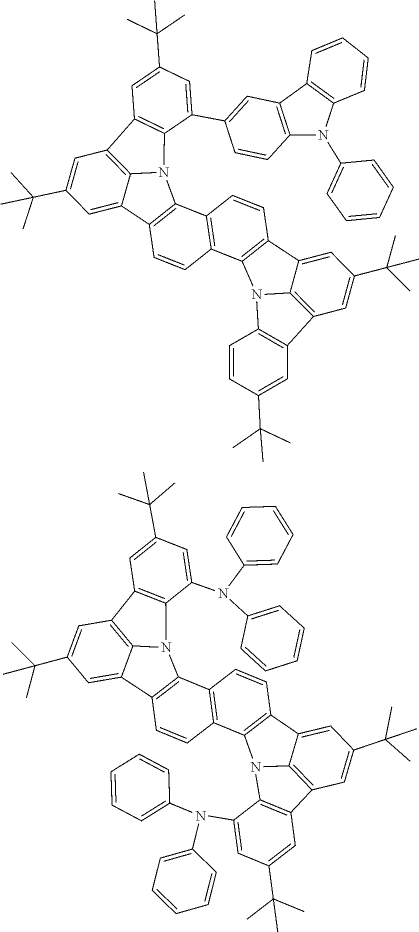

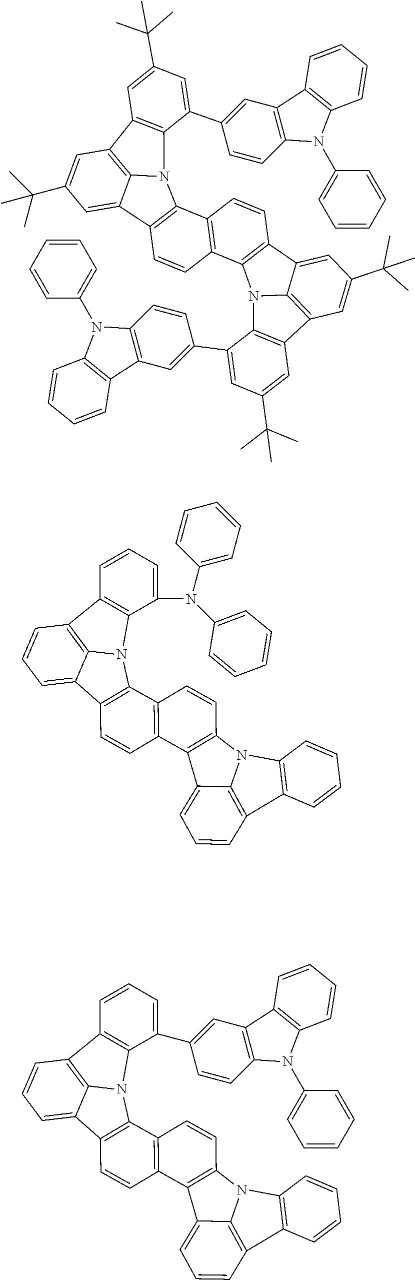 [SMILES: CC(C)(C)c1cc(-c2ccc3c(c2)c2ccccc2n3-c2ccccc2)c2c(c1)c1cc(C(C)(C)C)cc3c4ccc5c(ccc6c7cc(C(C)(C)C)cc8c9cc(C(C)(C)C)cc(-c%10ccc%11c(c%10)c%10ccccc%10n%11-c%10ccccc%10)c9n(c87)c65)c4n2c13 ; c1ccc(N(c2ccccc2)c2cccc3c4cccc5c6ccc7c(ccc8c7c7cccc9c%10ccccc%10n8c97)c6n(c23)c45)cc1 ; c1ccc(-n2c3ccccc3c3cc(-c4cccc5c6cccc7c8ccc9c(ccc%10c9c9cccc%11c%12ccccc%12n%10c%119)c8n(c45)c67)ccc32)cc1]

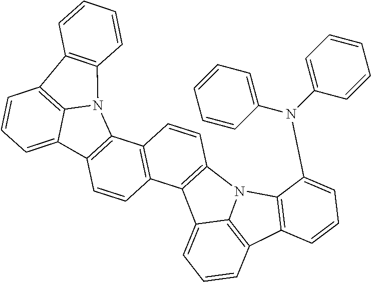 [SMILES: c1ccc(N(c2ccccc2)c2cccc3c4cccc5c6c7ccc8c9cccc%10c%11ccccc%11n(c8c7ccc6n(c23)c45)c%109)cc1]

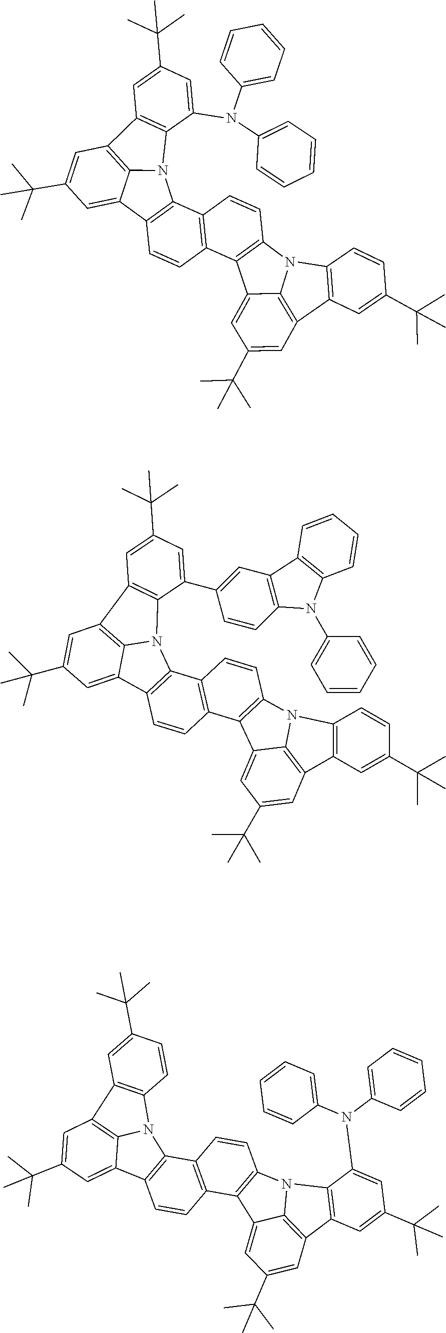

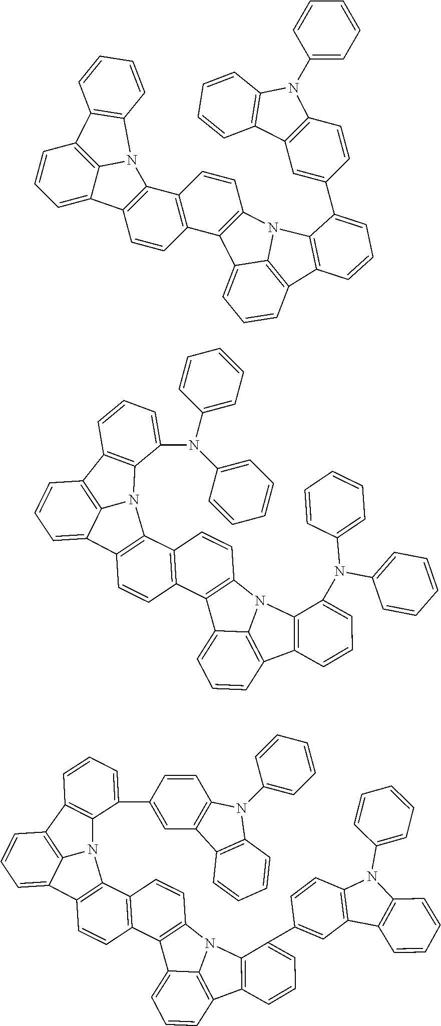

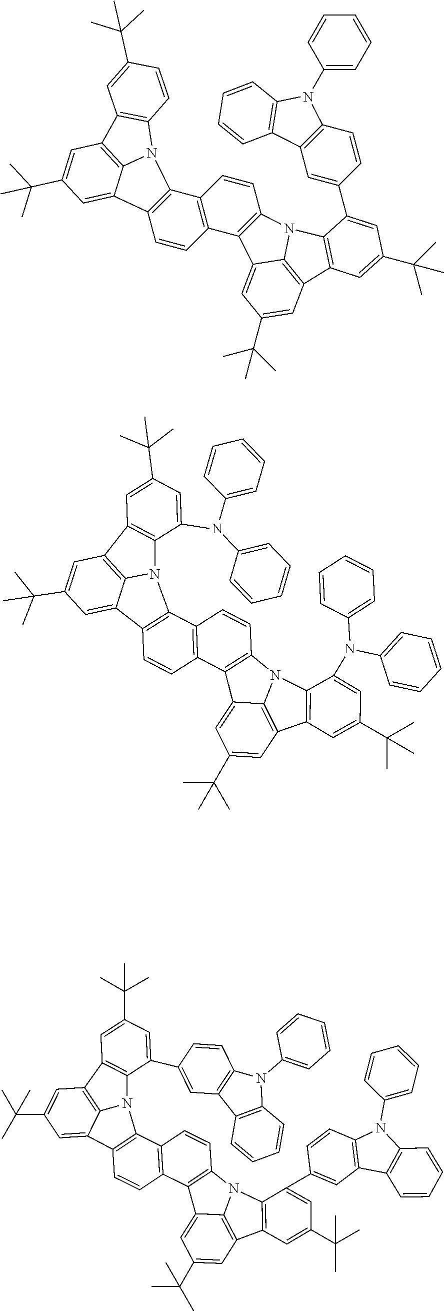

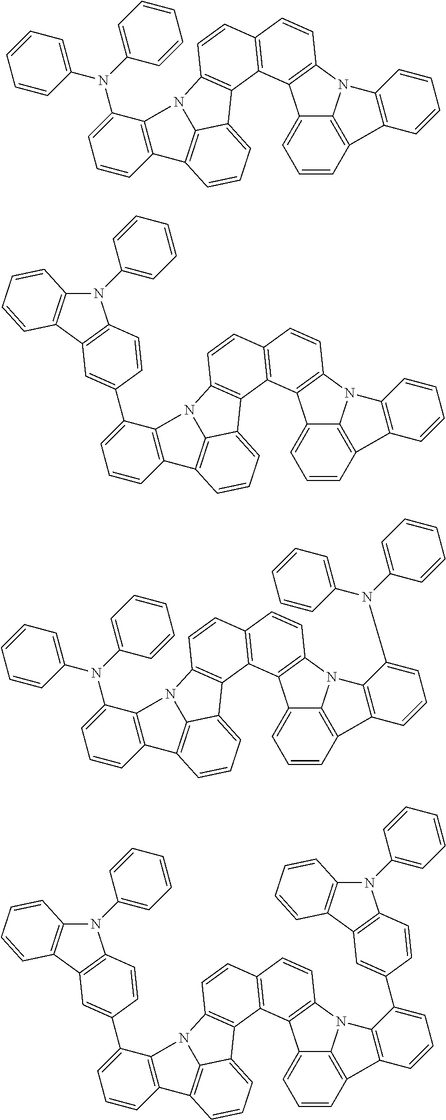

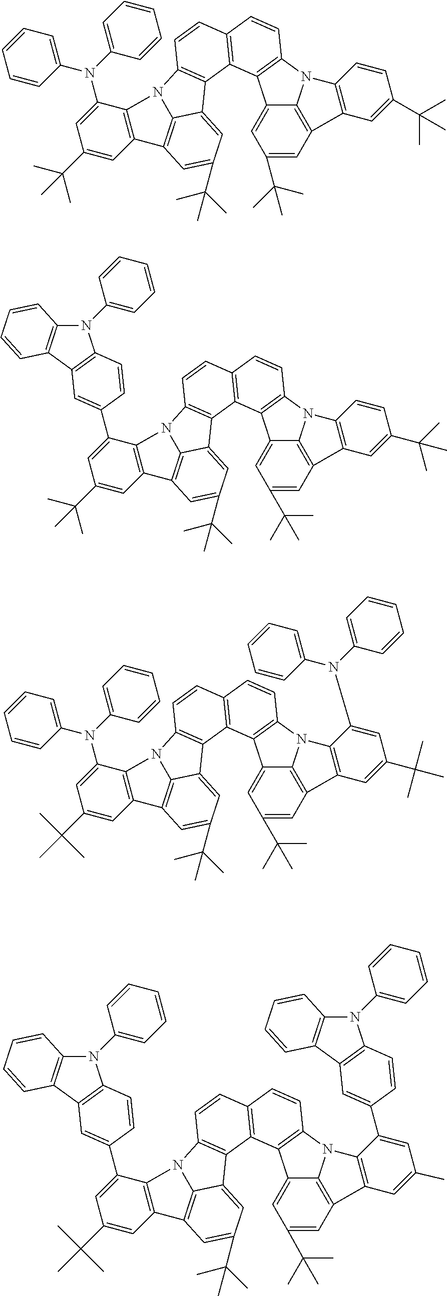

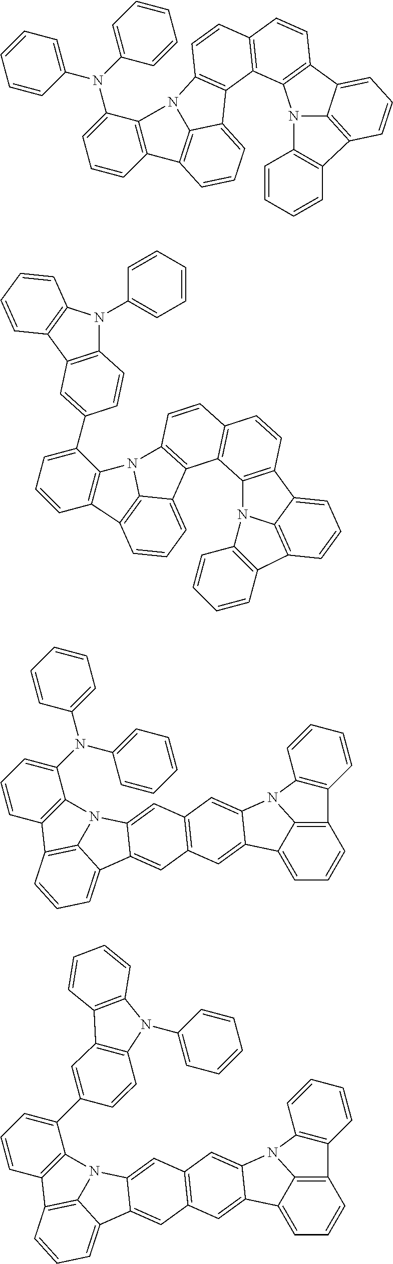

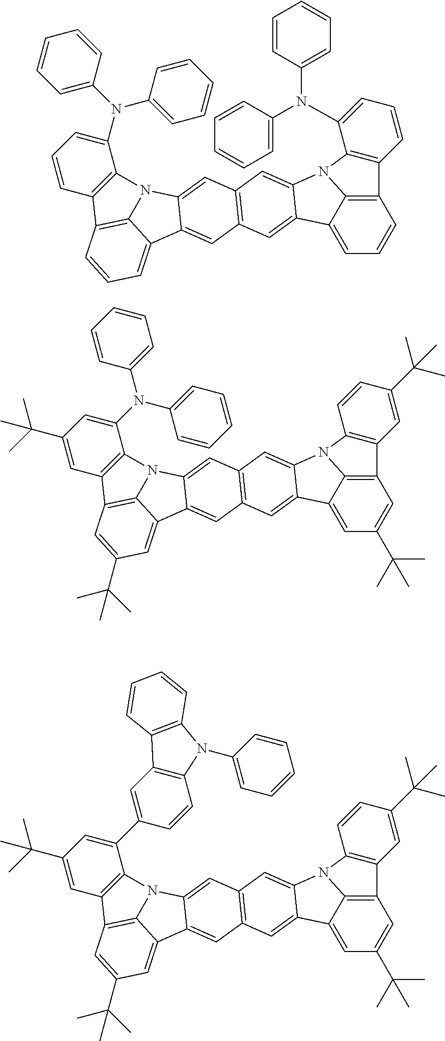

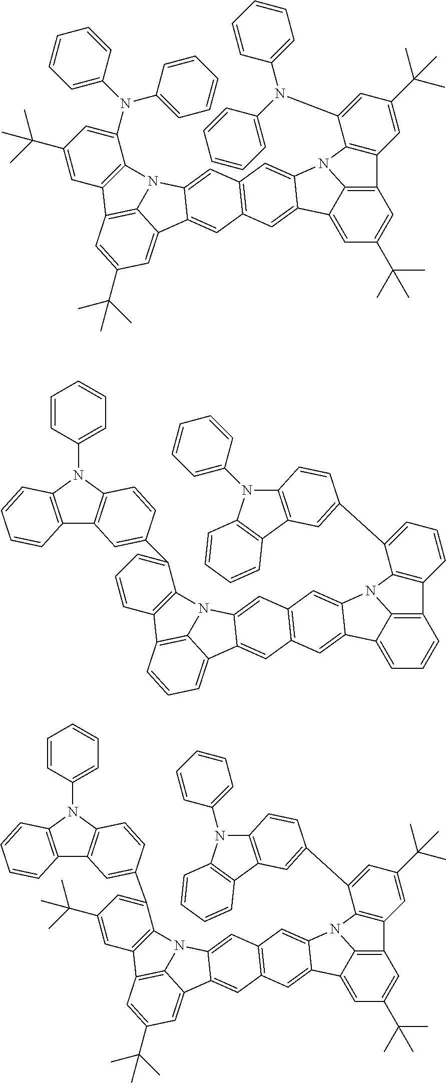 [SMILES: CC(C)(C)c1cc(N(c2ccccc2)c2ccccc2)c2c(c1)c1cc(C(C)(C)C)cc3c4cc5cc6c7cc(C(C)(C)C)cc8c9cc(C(C)(C)C)cc(N(c%10ccccc%10)c%10ccccc%10)c9n(c6cc5cc4n2c31)c78 ; c1ccc(-n2c3ccccc3c3cc(-c4cccc5c6cccc7c8cc9cc%10c%11cccc%12c%13cccc(-c%14ccc%15c(c%14)c%14ccccc%14n%15-c%14ccccc%14)c%13n(c%10cc9cc8n(c45)c76)c%11%12)ccc32)cc1 ; CC(C)(C)c1cc(-c2ccc3c(c2)c2ccccc2n3-c2ccccc2)c2c(c1)c1cc(C(C)(C)C)cc3c4cc5cc6c7cc(C(C)(C)C)cc8c9cc(C(C)(C)C)cc(-c%10ccc%11c(c%10)c%10ccccc%10n%11-c%10ccccc%10)c9n(c6cc5cc4n2c31)c78]

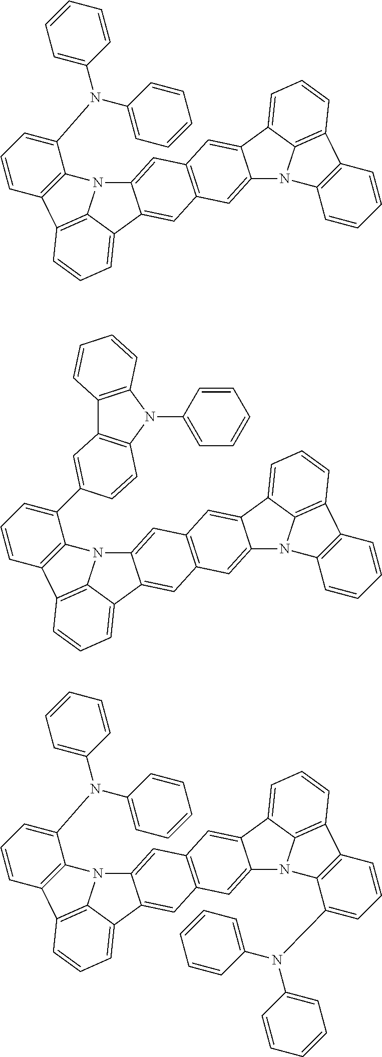

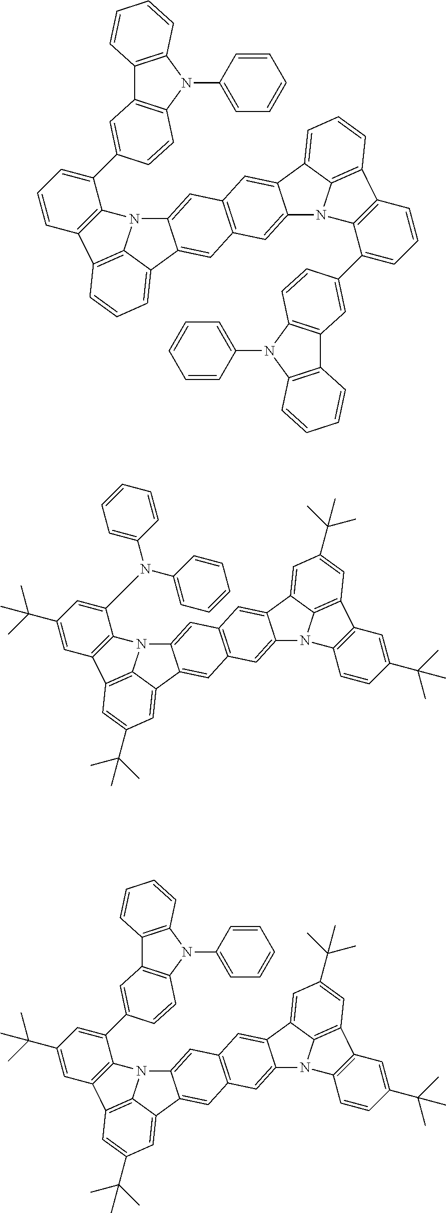

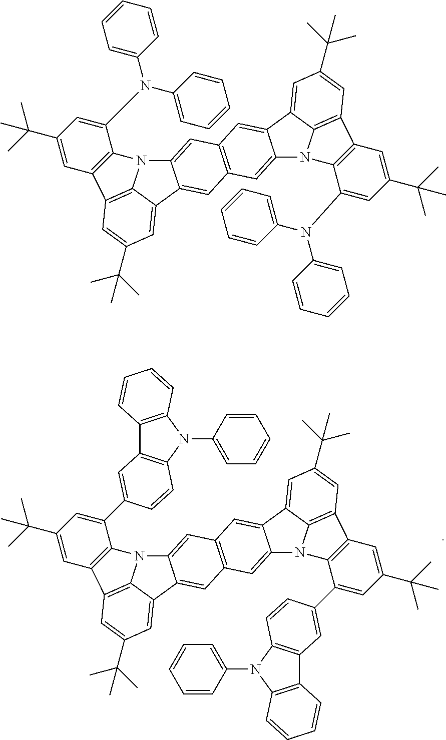

11. An organic light-emitting device comprising:
a first electrode;
a second electrode; and
an organic layer arranged between the first electrode and the second electrode and comprising an emission layer, wherein
the organic layer comprises the heterocyclic compound of claim 1.

12. The light-emitting device of claim 11, wherein the emission layer comprises the heterocyclic compound.

13. The light-emitting device of claim 12, wherein
the emission layer comprises a host and an emitter,
the host and the emitter are different from each other,
an amount of the host is greater than an amount of the emitter, and
the emitter comprises the heterocyclic compound.

14. The organic light-emitting device of claim 13, wherein the emission layer emits blue light.

15. The organic light-emitting device of claim 13, wherein the emitter is a fluorescent emitter, a delayed fluorescence emitter, or a combination thereof.

16. The organic light-emitting device of claim 12, wherein
the emission layer comprises a host, an emitter, and a sensitizer,
the host, the emitter, and the sensitizer are different from one another, an amount of the host is greater than an amount of the sensitizer,
an amount of the sensitizer is greater than an amount of the emitter, and
the emitter comprises the heterocyclic compound.

17. The organic light-emitting device of claim 16, wherein the emission layer emits blue light.

18. The organic light-emitting device of claim 16, wherein the emitter is a fluorescent emitter, a delayed fluorescence emitter, or a combination thereof.

19. The organic light-emitting device of claim 16, wherein the heterocyclic compound additionally satisfies Condition 5:
0 μs<T_{decay}(HC)<5 μs Condition 5
wherein, in Condition 5,
T_{decay}(HC) indicates a decay time of the heterocyclic compound.

20. An electronic apparatus comprising the organic light-emitting device of claim 11.
